(19) United States
(12) Patent Application Publication
(10) Pub. No.: US 2023/0105008 A1
(43) Pub. Date: Apr. 6, 2023

(54) METHODS AND COMPOSITIONS FOR IDENTIFYING CASTRATION RESISTANT NEUROENDOCRINE PROSTATE CANCER

(71) Applicants: Dana-Farber Cancer Institute, Inc., Boston, MA (US); Universita’ Degli Studi Di Trento, Trento (IT)

(72) Inventors: Himisha Beltran, Chestnut Hill, MA (US); Francesca Demichelis, Trento (IT); Gian Marco Franceschini, Aland Di Piave, BL (IT); Alessandro Romanel, Trento (IT)

(21) Appl. No.: 17/796,830

(22) PCT Filed: Feb. 8, 2021
(86) PCT No.: PCT/US21/17056

§ 371 Date: Aug. 1, 2022

Related U.S. Application Data
(60) Provisional application No. 62/975,009, filed on Feb. 11, 2020.

(51) Int. Cl. C12Q 1/6886

(52) U.S. Cl. CPC C12Q 1/6886; C12Q 2600/154; C12Q 2600/156; C12Q 2600/166; C12Q 1/6869

(57) ABSTRACT

The present invention is based on the identification of castration resistant neuroendocrine prostate cancer (CRPC-NE) features in the circulation, and relates to methods and compositions of identifying CRPC-NE patients by detecting these features.

### CROSS-REFERENCE TO RELATED APPLICATIONS

This application claims the benefit of priority to U.S. Provisional Application Ser. No. 62/975,009, filed on 11 Feb. 2020; the entire contents of said application is incorporated herein in its entirety by this reference.

### STATEMENT OF RIGHTS

This invention was made with government support under grant number P50 CA211024 awarded by the National Institutes of Health. The government has certain rights in the invention.

### BACKGROUND OF THE INVENTION

Prostate cancer is a leading cause of cancer death for men worldwide (Siegel, Miller, and Jemal (2019) CA Cancer J Clin 69:7-34). Despite significant advances in therapy for patients with metastatic disease, the emergence of treatment resistance remains a universal problem which ultimately contributes to the lethality of the disease. Prostate cancer is a cancer type that is driven by androgen receptor (AR) signaling, and several potent drugs targeting the AR are now commonly used to treat patients with advanced disease either in combination with gonadal androgen suppression therapy for metastatic hormone naive prostate cancer or in the castration resistant setting. Resistance to potent AR targeted drugs is still primarily mediated through AR-signaling (Watson, Arora, and Sawyers (2015) Nat Rev Cancer 15:701-711). However, an increasingly recognized proportion of tumors develop histologic transformation to a castration resistant neuroendocrine prostate cancer (CRPC-NE) phenotype as a mechanism of AR-independent treatment resistance (Beltran et al. (2019) Clin Cancer Res 25:6916-6924; Abida et al. (2019) Proc Natl Acad Sci USA 116:11428-11436; Aggarwal et al. (2018) J Clin Oncol JCO2017776880). Metastatic biopsies demonstrate morphologic features of small cell carcinoma, often with low or absent expression of the AR, downregulation of downstream AR-regulated markers such as prostate specific antigen (PSA), and expression of classical neuroendocrine markers (e.g., chromogranin, synaptophysin) (Epstein et al. (2014) Am J Surg Pathol 38:756-767; Beltran et al. (2011) Cancer Discov 1:487-495). The prognosis of CRPC-NE is poor due in part to late diagnosis and a lack of effective therapies (Aggarwal et al. (2018) J Clin Oncol JCO2017776880; Conteduca et al. (2019) Eur J Cancer 121:7-18). Similar to other poorly differentiated neuroendocrine carcinomas (Rickman et al. (2017) Nat Med 23:1-10; George et al. (2015) Nature 524:47-53), CRPC-NE frequently harbors genomic loss of RB1 and TP53 (Abida et al. (2019) Proc Natl Acad Sci USA 116:11428-11436; Epstein et al. (2014) Am J Surg Pathol 38:756-767; Beltran et al. (2011) Cancer Discov 1:487-495; Beltran et al. (2016) Nat Med 22:298-305; Tan et al. (2014) Clin Cancer Res 20:890-903). However, RB1 and TP53 loss-of-function alterations are not specific to CRPC-NE and also observed in a subset of castration resistant adenocarcinomas (Abida et al. (2019) Proc Natl Acad Sci USA 116:11428-11436). Prior clinical and preclinical studies have supported a transdifferentiation process whereby CRPC-NE evolves clonally from a luminal prostate adenocarcinoma precursor (Beltran et al. (2016) Nat Med 22:298-305; Zou et al. (2017) Cancer Discov 7:736-749; Ku et al. (2017) Science 355:78-83; Mu et al. (2017) Science 355:84-88; Park et al. (2018) Science 362:91-95). Early prostate cancer genomic alterations are retained, but other genomic and epigenetic alterations are acquired (Beltran et al. (2016) Nat Med 22:298-305; Ku et al. (2017) Science 355:78-83; Berger et al. (2019) J Clin Invest 130:3924-3940). How and when this lineage plasticity manifests in patients is not well understood and whether early detection of CRPC-NE could improve outcomes is not known.

Understanding patterns of tumor evolution that occur during prostate cancer progression and treatment resistance can inform tumor biology as well as identify novel strategies for combating resistance. Serial metastatic sampling in patients with prostate cancer during progression is challenging and is also not always representative of the heterogeneity of alterations that may exist across metastases in an individual. While rapid autopsy studies have pointed to limited intra-individual genomic (Kumar et al. (2016) Nat Med 22:369-378) and epigenomic (DNA methylation) (Yegnasubramanian et al. (2004) Cancer Res 64:1975-1986; Aryee et al. (2013) Sci Transl Med 5:169ra110) heterogeneity across anatomic sites of metastases at the time of lethal disease, circulating tumor DNA studies at earlier prostate cancer disease states indicates clonal dynamics that occur during tumor evolution with competing clones and subclones potentially contributing to resistance and progression (Carreira et al. (2014) Sci Transl Med 6:254ra125). Most studies to date have focused on castration resistant adenocarcinoma (CRPC-Adeno), and little is known regarding the underlying heterogeneity and tumor dynamics in the case of CRPC-NE evolution.

Loss of androgen receptor (AR) signaling dependence occurs in approximately 15-20% of treatment resistant prostate cancers, and this may manifest clinically as transformation from a prostate adenocarcinoma histology to a CRPC-NE. The diagnosis of CRPC-NE currently relies on a metastatic tumor biopsy, which is invasive for patients and sometimes challenging to diagnose due to morphologic heterogeneity. Accordingly, a great need exists for diagnostic, prognostic, and therapeutic methods regarding CRPC-NE.

### SUMMARY OF THE INVENTION

The present invention is based, at least in part, on the discovery that a targeted set combining genomic (e.g., TP53, RB1, CPLD, AR) and epigenomic (e.g., hypo- and hyper-methylation sites) alterations that was capable of identifying patients with CRPC-NE. These genomic and epigenomic alterations were detectable in the circulation and successfully applied to circulating tumor (ctDNA) for identifying patients with CRPC-NE.

In one aspect, a method of assessing whether a subject is afflicted with castration-resistant neuroendocrine prosate cancer (CRPC-NE) or at risk for developing CRPC-NE, the method comprising determining the presence or absence of one or more genomic or epigenomic alterations selected from: i) deletion or mutation of at least one biomarker listed in Table 1A; ii) gain or mutation of at least one biomarker listed in Table 1B; iii) hypermethylation of at least one genomic site listed in Table 1C; and iv) hypomethylation of at least one genomic site listed in Table 1D in the genomic DNA; wherein the presence of deletion or mutation of at least one biomarker listed in Table 1A, hypermethylation of at least one genomic site listed in Table 1C, and/or hypomethylation of at least one genomic site listed in Table 1D in the genomic DNA; and/or the absence of gain or mutation of at least one biomarker listed in Table 1B in the genomic DNA isolated from the biological sample indicates that the subject is afflicted with CRPC-NE or at risk for developing CRPC-NE, optionally obtaining a biological sample from the subject for the determination step, is provided.

Numerous embodiments are further provided that can be applied to any aspect of the present invention and/or combined with any other embodiment described herein. For example, in one embodiment, the deletion or mutation of at least one biomarker listed in Table 1A or the gain or mutation of at least one biomarker listed in Table 1B is detected by whole exome sequencing (WES). In another embodiment, the hypermethylation of at least one genomic site listed in Table 1C or hypomethylation of at least one genomic site listed in Table 1D is detected by whole genome bisulfite sequencing (WGBS). In still another embodiment, the deletion of at least one biomarker listed in Table 1A is a homozygous deletion, a heterozygous deletion, a copy number neutral loss, or an event defined by loss of one allele and gain of the other allele. In yet another embodiment, the mutation of at least one biomarker listed in Table 1A is a non-synonymous single-nucleotide variant (SNV). In another embodiment, the gain of at least one biomarker listed in Table 1B is a focal gain. In still another embodiment, the mutation of at least one biomarker listed in Table 1B is a non-synonymous single-nucleotide variant (SNV) (e.g., L702H or T878A of SEQ ID NO: 48). In yet another embodiment, the hypermethylation of at least one genomic site listed in Table 1C is defined by a higher methylation level than the site specific tissue-based threshold. In another embodiment, the hypomethylation of at least one genomic site listed in Table 1C is defined by a lower methylation level than the site specific tissue-based threshold. In still another embodiment, the threshold for each genomic site listed in Table 1C or 1D is a pre-determined threshold that discriminates between castration resistant prostate adenocarcinoma (CRPC-Adeno) and CRPC-NE tissue samples. In yet another embodiment, the method further comprises, after step (c), calculating a neuroendocrine prostate cancer (NEPC) score based on the presence or absence of one or more genomic or epigenomic features determined in step (c). In another embodiment, the method further comprises comparing the NEPC score to a control, wherein a higher NEPC score compared to the control indicates that the subject is afflicted with CRPC-NE or at risk for developing CRPC-NE. In another embodiment, the control is a reference value. In still another embodiment, the control is a NEPC score determined from a control sample. In yet another embodiment, the control sample is obtained from a subject without CRPC-NE, or a member of the same species to which the subject belongs without CRPC-NE. In another embodiment, the control sample is obtained from a subject with castration resistant prostate adenocarcinoma (CRPC-Adeno). In still another embodiment, the sample is selected from the group consisting of organs, tissue, body fluids and cells. In yet another embodiment, the body fluid is selected from the group consisting of whole blood, serum, plasma, sputum, spinal fluid, lymph fluid, skin secretions, respiratory secrections, intestinal secretions, genitourninary tract secretions, tears, milk, buccal scrape, saliva, cerebrospinal fluid, urine, and stool. In another embodiment, the sample is whole blood, serum or plasma. In still another embodiment, cell-free DNA (cfDNA) or circulating tumore DNA (ctDNA) isolated from plasma is used for the determination. In still another embodiment, genomic DNA isolated from a tumor cell or tissue is used for the determination. In yet another embodiment, the method further comprises comparing additional biomarkers for CRPC-NE, such as a biomarker selected from the group consisting of AR, a downstream AR-regulated marker, and a classical neuroendocrine marker. In another embodiment, the downstream AR-regulated marker is selected from the group consisting of prostate specific antigen (PSA), NKX3.1, and TMPRSS2. In still another embodiment, the classical neuroendocrine marker is selected from the group consisting of chromogranin, synaptophysin, neuron specific enolase, and CD56. In yet another embodiment, the method further comprises detecting morphological features of a tumor biopsy from the subject. In another embodiment, the subject is afflicted with castration-resistant prostate cancer (CRPC). In still another embodiment, the subject is resistant to an androgen receptor (AR)-directed therapy. In yet another embodiment, the method further comprises administering to the subject an anti-cancer therapy other than an AR-targeted therapy as a single agent if the subject is afflicted with CRPC-NE or at risk for developing CRPC-NE. In another embodiment, the anti-cancer therapy is selected from the group consisting of an epgenitic modifier, targeted therapy, chemotherapy, radiation therapy, and/or hormonal therapy, optionally wherein the anti-cancer therapy comprises an AR-targeted therapy. In still another embodiment, the anti-cancer therapy is administered to the subject in combination with the AR-targeted therapy, optionally wherein the anti-cancer therapy is administered before, after, or concurrently with the AR-targeted therapy. In yet another embodiment, the targeted therapy is an immunotherapy. In another embodiment, the immunotherapy is cell-based. In still another embodiment, the immunotherapy comprises a cancer vaccine and/or virus. In yet another embodiment, the immunotherapy inhibits an immune checkpoint, such as an immune checkpoint selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR. In another embodiment, the immune checkpoint is PD1, PD-L1, or CTLA-4. In still another embodiment, the anti-cancer therapy is a platinum-based chemotherapy.

In another aspect, a method for monitoring the progression of CRPC in a subject, the method comprising: a) detecting in a subject sample at a first point in time the presence or absence of one or more genomic or epigenomic features selected from: i) deletion or mutation of at least one biomarker listed in Table 1A; ii) gain or mutation of at least one biomarker listed in Table 1B; iii) hypermethylation of at least one genomic site listed in Table 1C; and iv) hypomethylation of at least one genomic site listed in Table 1D; b) calculating a first NEPC score based on the presence or absence of one or more genomic or epigenomic features determined in step a); c) repeating step a) at a subsequent point in time; d) calculating a second NEPC score based on the presence or absence of one or more genomic or epigenomic features determined in step c); and e) comparing the NEPC scores determined in steps b) and d), and therefrom monitoring the progression of CRPC in the subject, is provided.

In still another aspect, a method of assessing the efficacy of an agent for treating CRPC-NE in a subject, the method comprising: a) detecting in a subject sample at a first point in time the presence or absence of one or more genomic or epigenomic features selected from: i) deletion or mutation of at least one biomarker listed in Table 1A; ii) gain or mutation of at least one biomarker listed in Table 1B; iii) hypermethylation of at least one genomic site listed in Table 1C; and iv) hypomethylation of at least one genomic site listed in Table 1D; b) calculating a first NEPC score based on the presence or absence of one or more genomic or epigenomic features determined in step a); c) repeating step a) during at least one subsequent point in time after administration of the agent; d) calculating a second NEPC score based on the presence or absence of one or more genomic or epigenomic features determined in step c); and e) comparing the NEPC scores determined from steps b) and d), wherein a higher NEPC score determined in the subsequent sample, relative to the sample at the first point in time, indicates that the agent does not treat CRPC-NE in the subject; and wherein a lower NEPC score determined in the subsequent sample, relative to the sample at the first point in time, indicates that the agent treats CRPC-NE in the subject, is provided.

As described above, numerous embodiments are further provided that can be applied to any aspect of the present invention and/or combined with any other embodiment described herein. For example, in one embodiment, the subject has undergone treatment, completed treatment, and/or is in remission for CRPC-NE between the first point in time and the subsequent point in time. In another embodiment, the first and/or at least one subsequent sample is selected from the group consisting of ex vivo and in vivo samples. In still another embodiment, the first and/or at least one subsequent sample is obtained from an animal model of CRPC-NE. In yet another embodiment, the first and/or at least one subsequent sample is a portion of a single sample or pooled samples obtained from the subject. In another embodiment, the sample comprises cells, cell lines, histological slides, paraffin embedded tissue, fresh frozen tissue, fresh tissue, biopsies, blood, plasma, serum, buccal scrape, saliva, cerebrospinal fluid, urine, stool, mucus, bone marrow, peritumoral tissue, and/or intratumoral tissue obtained from the subject. In another embodiment, the sample is whole blood, serum or plasma. In still another embodiment, the sample is a tumor cell or tissue, and genomic DNA is isolated from the plasma and used for detecting the presence or absence of one or more genomic or epigenomic features. In still another embodiment, the sample is plasma, and cell-free DNA (cfDNA) or circulating tumore DNA (ctDNA) is isolated from the plasma and used for detecting the presence or absence of one or more genomic or epigenomic features.

In yet another aspect, a cell-based assay for screening for agents that have a cytotoxic or cytostatic effect on a CRPC-NE cancer cell comprising, contacting the CRPC-NE cancer cell with a test agent, and determining the ability of the teat agent: i) to inhibit deletion or mutation of at least one biomarker listed in Table 1A; ii) to induce gain or mutation of at least one biomarker listed in Table 1B; iii) to decrease the methylation level at least one genomic site listed in Table 1C; and/or iv) to increase the methylation level of at least one genomic site listed in Table 1D in the subject sample, is provided.

As described above, numerous embodiments are further provided that can be applied to any aspect of the present invention and/or combined with any other embodiment described herein. For example, in one embodiment, the step of contacting occurs in vivo, ex vivo, or in vitro. In another embodiment, the assay further comprises administering the test agent to an animal model of CRPC-NE.

In an other aspect, a kit for assessing the ability of a agent to treat CRPC-NE, the kit comprising a reagent for assessing the presence or absence of one or more genomic or epigenomic features selected from: i) deletion or mutation of at least one biomarker listed in Table 1A; ii) gain or mutation of at least one biomarker listed in Table 1B; iii) hypermethylation of at least one genomic site listed in Table 1C; and iv) hypomethylation of at least one genomic site listed in Table 1D, is provided.

In still another aspect, a kit for assessing whether a subject is afflicted with CRPC-NE or at risk for developing CRPC-NE, the kit comprising a reagent for assessing the presence or absence of one or more genomic or epigenomic features selected from: i) deletion or mutation of at least one biomarker listed in Table 1A; ii) gain or mutation of at least one biomarker listed in Table 1B; iii) hypermethylation of at least one genomic site listed in Table 1C; and iv) hypomethylation of at least one genomic site listed in Table 1D, is provided.

In yet another aspect, a method of treating a subject afflicted with CRPC-NE comprising administering to the subject a therapeutically effective amount of an agent that modulates the presence or absence of one or more genomic or epigenomic features selected from: i) deletion or mutation of at least one biomarker listed in Table 1A; ii) gain or mutation of at least one biomarker listed in Table 1B; iii) hypermethylation of at least one genomic site listed in Table 1C; and iv) hypomethylation of at least one genomic site listed in Table 1D, is provided.

As described above, numerous embodiments are further provided that can be applied to any aspect of the present invention and/or combined with any other embodiment described herein. For example, in one embodiment, the agent inhibits deletion or mutation of at least one biomarker listed in Table 1A, thereby treating a subject afflicted with CRPC-NE. In another embodiment, the agent induces gain or mutation of at least one biomarker listed in Table 1B, thereby treating a subject afflicted with CRPC-NE. In still another embodiment, the agent decreases the methylation level at least one genomic site listed in Table 1C, thereby treating a subject afflicted with CRPC-NE. In yet another embodiment, the agent increases the methylation level at least one genomic site listed in Table 1D, thereby treating a subject afflicted with CRPC-NE. In another embodiment, the agent is an epigenetic modifier, such as an EZH2 inhibitor. In still another embodiment, the method further comprises administering to the subject an immunotherapy and/or cancer therapy, optionally wherein the immunotherapy and/or cancer therapy is administered before, after, or concurrently with the agent. In yet another embodiment, the immunotherapy is cell-based. In another embodiment, the immunotherapy comprises a cancer vaccine and/or virus. In still another embodiment, the immunotherapy inhibits an immune checkpoint, such as an immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR. In yet another embodiment, the immune checkpoint is PD1, PD-L1, or CD47. In another embodiment, the cancer therapy is selected from the group consisting of radiation, a radiosensitizer, and a chemotherapy. In another embodiment, the cancer therapy is a platinum-based chemotherapy. In still another embodiment, the deletion or mutation of at least one biomarker listed in Table 1A or the gain or mutation of at least one biomarker listed in Table 1B is detected by whole exome sequencing (WES). In yet another embodiment, the hypermethylation of at least one genomic site listed in Table 1C or hypomethylation of at least one genomic site listed in Table 1D is detected by whole genome bisulfite sequencing (WGBS). In another embodiment, the deletion of at least one biomarker listed in Table 1A is a homozygous deletion, a heterozygous deletion, a copy number neutral loss, or an event defined by loss of one allele and gain of the other allele. In still another embodiment, the mutation of at least one biomarker listed in Table 1A is a non-synonymous single-nucleotide variant (SNV). In yet another embodiment, the gain of at least one biomarker listed in Table 1B is a focal gain. In another embodiment, the mutation of at least one biomarker listed in Table 1B is a non-synonymous single-nucleotide variant (SNV) (e.g., L702H or T878A of SEQ ID NO: 48). In still another embodiment, the hypermethylation of at least one genomic site listed in Table 1C is defined by a higher methylation level than the site specific tissue-based threshold. In yet another embodiment, the hypomethylation of at least one genomic site listed in Table 1C is defined by a lower methylation level than the site specific tissue-based threshold. In another embodiment, the threshold for each genomic site listed in Table 1C or 1D is the threshold that best discriminates between castration resistant prostate adenocarcinoma (CRPC-Adeno) and CRPC-NE tissue samples by Receiver operating characteristic (ROC) curve analysis. In still another embodiment, the agent is administered in a pharmaceutically acceptable formulation. In yet another embodiment, the subject is an animal model of CRPC-NE. In another embodiment, the animal model is a rodent model. In still another embodiment, the subject is a mammal, such as a mouse or a human.

In yet another aspect, a method is provided for assessing whether a subject is afflicted with or at risk for developing castration-resistant neuroendocrine prostate cancer (CRPC-NE) or castration-resistant adenocarcinoma prostate cancer (CRPC-Adeno), the method comprising determining the presence or absence of one or more genomic or epigenomic alterations in at least one biomarker listed in Table 14, wherein the presence one or more genomic or epigenomic alterations listed in Table 14 in the genomic DNA isolated from the biological sample indicates that the subject is afflicted with CRPC-NE or CRPC-Adeno or at risk for developing CRPC-NE or CRPC-Adeno, optionally obtaining a biological sample from the subject for the determination step.

As described above, numerous embodiments are further provided that can be applied to any aspect of the present invention and/or combined with any other embodiment described herein. For example, in one embodiment, the one or more genomic or epigenomic alterations listed in Table 14 comprises a mutation detected by whole exome sequencing (WES). In another embodiment, the one or more genomic or epigenomic alterations listed in Table 14 comprises hypermethylation or hypomethylation of at least one genomic site listed in Table 14 detected by whole genome bisulfite sequencing (WGBS). In still another embodiment, the one or more genomic or epigenomic alterations listed in Table 14 comprise a homozygous deletion, a heterozygous deletion, a copy number neutral loss, or an event defined by loss of one allele and gain of the other allele. In yet another embodiment, the one or more genomic or epigenomic alterations listed in Table 14 is a non-synonymous single-nucleotide variant (SNV). In one embodiment, the one or more genomic or epigenomic alterations listed in Table 14 is a focal gain of at least one biomarker listed in Table 14. In another embodiment, the one or more genomic or epigenomic alterations listed in Table 14 is a non-synonymous single-nucleotide variant (SNV). In still another embodiment, the hypermethylation of at least one genomic site listed in Table 14 is defined by a higher methylation level than the site specific tissue-based threshold. In yet another embodiment, the hypomethylation of at least one genomic site listed in Table 14 is defined by a lower methylation level than the site specific tissue-based threshold. In one embodiment, the threshold for each genomic site listed in Table 14 is a pre-determined threshold that discriminates between castration resistant prostate adenocarcinoma (CRPC-Adeno) and CRPC-NE tissue samples. In another embodiment, the method further comprises after step (c), based on the presence or absence of one or more genomic or epigenomic features determined in step (c). In still another embodiment, the method further comprises comparing the NEPC score to a control, wherein a higher NEPC score compared to the control indicates that the subject is afflicted with CRPC-NE or at risk for developing CRPC-NE. In yet another embodiment, the control is a reference value. In one embodiment, the control is a NEPC score determined from a control sample. In another embodiment, the control sample is obtained from a subject without CRPC-NE, or a member of the same species to which the subject belongs without CRPC-NE. In still another embodiment, the control sample is obtained from a subject with castration resistant prostate adenocarcinoma (CRPC-Adeno). In yet another embodiment, the sample is selected from the group consisting of organs, tissue, body fluids and cells. In one embodiment, the body fluid is selected from the group consisting of whole blood, serum, plasma, sputum, spinal fluid, lymph fluid, skin secretions, respiratory secrections, intestinal secretions, genitourninary tract secretions, tears, milk, buccal scrape, saliva, cerebrospinal fluid, urine, and stool. In another embodiment, the sample is whole blood, serum or plasma. In still another embodiment, cell-free DNA (cfDNA) or circulating tumore DNA (ctDNA) isolated from plasma is used for the determination. In yet another embodiment, genomic DNA isolated from a tumor cell or tissue is used for the determination. In one embodiment, the method

further comprises comparing additional biomarkers for CRPC-NE. In still another embodiment, the additional biomarker is selected from the group consisting of AR, a downstream AR-regulated marker, and a classical neuroendocrine marker. In one embodiment, the downstream AR-regulated marker is selected from the group consisting of prostate specific antigen (PSA), NKX3.1, and TMPRSS2. In another embodiment, the classical neuroendocrine marker is selected from the group consisting of chromogranin, synaptophysin, neuron specific enolase, and CD56. In still another embodiment, the method further comprises detecting morphological features of a tumor biopsy from the subject. In yet another embodiment, the subject is afflicted with castration-resistant prostate cancer (CRPC). In another embodiment, the subject is resistant to an androgen receptor (AR)-directed therapy. In still another embodiment, the method further comprises administering to the subject an anti-cancer therapy other than an AR-targeted therapy as a single agent if the subject is afflicted with CRPC-NE or at risk for developing CRPC-NE. In yet another embodiment, the anti-cancer therapy is selected from the group consisting of an epgenitic modifier, targeted therapy, chemotherapy, radiation therapy, and/or hormonal therapy, optionally wherein the anti-cancer therapy comprises an AR-targeted therapy. In one embodiment, the anti-cancer therapy is administered to the subject in combination with the AR-targeted therapy, optionally wherein the anti-cancer therapy is administered before, after, or concurrently with the AR-targeted therapy. In another embodiment, the targeted therapy is an immunotherapy. In yet another embodiment, the immunotherapy is cell-based. In one embodiment, the immunotherapy comprises a cancer vaccine and/or virus. In another embodiment, the immunotherapy inhibits an immune checkpoint, such as a checkpoint selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR. In one embodiment, the immune checkpoint is PD1, PD-L1, or CTLA-4. In one embodiment, the anti-cancer therapy is a platinum-based chemotherapy.

### BRIEF DESCRIPTION OF THE DRAWINGS

FIG. 1A-FIG. 1D show frequencies of somatic aberrations in advanced prostate cancer driver genes. FIG. 1A shows a schematic of study cohort. FIG. 1B shows whole exome sequencing (WES) segmented data for study cohort. WES segmented data are shown raw (inset) and ploidy and tumor content (TC) adjusted. FIG. 1C shows a distribution of somatic copy number loss and SNVs in CRPC-Adeno and CRPC-NE ctDNA and tumor tissue samples. Loss events include homozygous deletions (HomDel), heterozygous deletions (HetDel), copy number neutral losses (CNNL) and events defined by loss of one allele and gain of the other allele (Del|Gain). FIG. 1D shows AR somatic aberrations status in CRPC-Adeno, CRPC-NE and HNPC plasma and tumor tissue samples, ordered based on serial dates of collection. AR gain, focal gain and SNV (L702H and T878A positional pileup calls) are shown together with sample ploidy and tumor class. Statistics are reported in Table 8.

FIG. 2 shows a distribution of tumor content (TC), tumor ploidy, copy number aberration fraction (CNAF) and total number of somatic SNVs computed from genomic data across plasma and tumor tissue samples and across sample types. Reported p-values are computed using two-tailed Wilcoxon-Mann-Whitney test. Pie-charts represent for each genomic variable the fraction of samples for which (white portion) the value was able to estimate among, respectively, plasma and tissue samples.

FIG. 3A-FIG. 3D show analysis of plasma genomics and clinical variable associations. FIG. 3A shows a distribution of tumor content (TC) estimation in plasma among patients with different type and number of metastasis type. FIG. 3B shows a correlation of TC estimations in plasma and time to plasma collection from metastasis and number of therapy lines before plasma collection. FIG. 3C shows a correlation of TC estimations in plasma and NEPC clinical markers (PSA=prostate specific antigen, NSE=neuron-specific enolase, CGA=chromogranin A) and distribution of NEPC markers across patient's class at plasma collection. FIG. 3D shows a distribution of TC, copy number aberration fraction (CNAF), number of SNVs (outliers not shown) and number of missense SNVs (outliers not shown) across patients with or without chemotherapy treatment prior to plasma collection. Reported p-values are computed using two-tailed Wilcoxon-Mann-Whitney test or Pearson correlation.

FIG. 4A-FIG. 4D show association of plasma tumor content and plasma prostate cancer driver genes with overall survival and progression-free survival. FIGS. 4A and 4B show overall survival and radiographic progression-free survival for high versus low tumor content (TC) estimations (FIG. 4A) and for AR CN neutral versus AR focal gain (FIG. 4B); univariate analysis results only and multivariate (controlling for TC) analysis are shown in the inset. FIGS. 4C and 4D show the overall survival for lesions in TP53 and/or RB1 (FIG. 4C) and for BRCA1, BRCA2, ATM (FIG. 4D); univariate and multivariate analysis are shown in the insets. All analyses are reported for the entire cohort (left panel), for CRPC-Adeno (center panel) and for CRPC-NE (right panel). Univariate overall survival and progression-free survival analyses were performed using the Kaplan-Meier estimator (log-rank test). Multivariate overall and progression-free survival analyses were performed using a proportional hazard model with stepwise model selection by Akaike information criterion using forward and backward directions.

FIG. 5 shows the landscape of somatic aberrations in plasma and tissue samples across CRPC-Adeno (top panel), CRPC-NE (middle panel), and HNPC (bottom panel) classes. Grey cells in oncoprints represent either wild-type or absence of available call. Reported gene aberration fractions are computed excluding all samples for which no calls are available neither for SNVs nor for SCNAs.

FIG. 6A-FIG. 6D show similarity of somatic aberration profiles across plasma and metastatic tumor tissue samples. FIG. 6A shows intra-patient somatic copy number aberrations (SCNAs, left, Loss and Gain) and single nucleotide variants (SNVs, right) similarity across metastatic biopsies stratified by patient's tumor class at plasma collection (Table 10). FIG. 6B shows inter- and intra-patient measures of SCNAs similarity per site and across sites of metastasis. FIG. 6C shows intra-patient loss and gain SCNAs similarities (left) and SNVs (right) similarities (fraction of SNVs in plasma detected also in tissues and fraction of SNVs in tissues detected also in plasma) across tissue and plasma samples stratified by patient's tumor class at plasma collection. Only samples of patients with estimated ctDNA >10% are considered. The same trends are also obtained with more restrictive filter on TC>50%. FIG. 6D shows SCNAs similarity among plasma and tumor tissue samples of patient WCMO (left); Private and shared SNVs comparison between WCMO plasma and selected tumor tissue samples. Reported p-values are computed using two-tailed Wilcoxon-Mann-Whitney test.

FIG. 7 shows clonality divergence as assessed for tissue and plasma study cohort samples. Distribution of the clonality diverge index in tumor tissue and plasma samples across patients classified as CRPC-Adeno and CRPC-NE at plasma collection. Reported p-values are computed using two-tailed Wilcoxon-Mann-Whitney test.

FIG. 8A-FIG. 8B show allele-specific copy number quantification of matched plasma and tumor tissue samples. FIG. 8A shows patient data demonstrating almost identical genomic status between a metastasis and a plasma sample, indicating high homogeneity among all patient's metastases. The lymph node metastasis of patient WCM198 and the plasma sample were collected at 4 days apart. Polygons include cancer genes with same allele-specific copy number; red dots correspond to reported gene names. FIG. 8B shows patient data with heterogeneous profiles. Patient WCM183 liver metastasis was obtained 13 days prior to the plasma sample. Blue indicates genes with different allele-specific copy number.

FIG. 9A-FIG. 9B show patient data. FIG. 9A shows patient data demonstrating almost identical genomic status between a metastasis and a plasma sample, indicating high homogeneity among all patients' metastases. Polygons include cancer genes with same allele-specific copy number. FIG. 9B shows patient data with heterogeneous profiles. Blue indicates genes with different allele-specific copy number.

FIG. 10A-FIG. 10B show heterogeneity of somatic aberration profiles in multiple plasma and tumor tissue sample series. FIG. 10A shows somatic-copy number aberrations (SCNAs) and single nucleotide variants (SCNAs) similarities among plasma and tumor tissue samples of patient WCM161. Samples are ordered by date of collection with date differences reported in days. SNVs similarity is an asymmetric measure based on set inclusion and the complete matrix is shown. SCNAs similarity is instead a symmetric measure based on the Jaccard coefficient and hence information is shown without redundancy. FIG. 10B shows results of a genomic analysis of WCM185 and WMC14 multi-sample series. The panel reports: allele-specific copy number of a selection of advanced prostate cancer driver genes (HomDel=homozygous deletion, HetDel=heterozygous deletion, CNNL=copy number neutral loss, Del|Gain=events defined by loss of one allele and gain of the other allele); AR L702H and AR T878A SNVs (determined by pileup analysis); tumor content estimations; ploidy state estimation; normalized SNVs load, with values representing the ratio between the sample's SNVs number and the sample type (plasma/tumor tissue) median SNVs number. Samples are ordered by date of collection (intervals shown).

FIG. 11 shows SCNAs having similarity among plasma and tumor tissue samples of patients with multiple tissue biopsies and plasma tumor content greater or equal than 10%.

FIG. 12A-FIG. 12E show that differential methylation signal is detected in circulation of patients. FIG. 12A shows a PAMES purity estimation of plasma WGBS plasma and PBMC samples. Top 10 most informative hypermethylated CpG Island were used. FIG. 12B shows results of a Ward's hierarchical clustering of 25 samples using ‘1—Pearson's correlation coefficient’ as distance measure. The annotation tracks include information on sample tumor purity and on the site of the relative sequenced tissue biopsy for all tissue samples and on the presence or absence of lymph node, bone or visceral metastases in the corresponding patient for the plasma samples. FIG. 12C shows results of evaluation of differentially methylated regions (DMRs) concordance in matched plasma and tissue samples. Two distinct DMR sets were nominated applying Rocker-Meth algorithm on tissue samples and single sample z-scores were computed for each DMR. A comparison between values detected in ctDNA and tissue biopsy and results are reported for three representative patients: WCM90, CRPC-Adeno; WCMO, CRPC-NE; WCM119, CRPC-Adeno with radiographic progression on enzalutamide, PSA 3.5 and elevated serum NSE. Color density is proportional to point density to the power of ¼ to improve visualization. First-order linear regression R squared is reported. FIG. 12D shows a comparison of average absolute z-score based on CRPC-NE|CRPC-Adeno DMR in plasma samples from this cohort and from a set of patients treated with abiraterone acetate reported in Gordevicius et al. (2018) Clin Cancer Res 24:3317-3324. To maximize the compatibility of clinical history and disease stage, only end-of-treatment samples with an estimated tumor content greater than 10% were included. Significance was assessed using two-tailed unpaired Wilcoxon-Mann-Whitney test without continuity correction. FIG. 12E shows plotted NEPC feature scores as assessed in plasma data of CRPC-Adeno and CRPC-NE patients.

FIG. 13A-FIG. 13E show patient data. FIG. 13A shows a genome-wide comparison of methylation patterns in CRPC-NE and CRPC-Adeno as measured in plasma and in tissue samples. Differences averaged betas are plotted. Red line shows interpolation by the lowess function; R is the Pearson's correlation coefficient. FIG. 13B shows tumor fraction estimates from patient circulating material by a scatter plot of the tumor purity estimates by a methylation based (PAMES on WGBS data) versus a genomic based approach (CLONET on WES). Colors refer to the pathology classification. R is the Pearson's correlation coefficient. FIG. 13C shows beta values in plasma and tissue samples across portions of four genes of interest. ASXL3 and SPDEF were included in the NEPC classifier from Beltran and Prandi et al. CDH2 and INSM1 are associated with sites used in the NEPC feature score. Lines are fitted to CRPC-Adeno (pink) and CRPC-NE (purple) samples medians of single CpG sites using loess function. Plasma CpG sites were filtered keeping only those presenting at least 2 measurements for each class. FIG. 13D shows a comparison of average absolute z-score measured in tissue samples (Beltran et al. (2016) Nat Med 22:298-305) and plasma samples (this cohort) for the two target DMR sets reported in FIG. 14. P-values are calculated with unpaired two-sided Wilcoxon-Mann-Whitney test. FIG. 13E shows box plots of the distribution of beta values in ctDNA samples for sites identified as differentially methylated in CRPC-NE vs CRPC-Adeno in tissue samples (hypo: AUC<0.2; hyper: AUC>0.8). P-value are calculated by Wilcoxon-Mann-Whitney test.

FIG. 14 shows a proposed model of prostate cancer progression towards CRPC-NE. Metastatic prostate cancer lesions harbor shared alterations often traceable back to a primary tumor clone, supporting a monoclonal origin of metastatic prostate cancer. Tumors acquire alterations with disease progression and treatment resistance, and these alterations can be subclonal. Polyclonal spread in later stages further leads to intra-patient heterogeneity. During the transition towards CRPC-NE, there is likely selection of a dominant clone that persists and dominates. DNA methylation profiles dramatically shift with CRPC-NE.

FIG. 15A-FIG. 15E show results of input DNA titration experiments for whole exome sequencing and deduplication analysis. FIG. 15A shows copy number profiles correlation for a set of cancer genes assessed from processed sequencing data from a series of titration experiment. The sample was selected based on tumor volume/tumor content. Data are shown from libraries built starting from 50 ng, 20 ng, 10 ng, or 5 ng, and processed with or without a filter to remove read duplicates (DR). FIG. 15B shows a genome-wide view of copy number profiles of previous data is shown together with matched tumor tissue biopsy data. FIG. 15C shows the percentiles of read duplicates distribution across plasma and germline samples and across different coverage intervals.

FIG. 15D shows distributions of germline and plasma mean coverage across ad hoc de-duplication strategies. FIG. 15E shows a comparison of copy number profiles for a set of cancer genes obtained from sequencing data including read duplicates and upon deduplication using different strategies. See working example 1 herein. (SE=single end sequencing protocol, PE=paired end sequencing protocol, DR=inclusion of read duplicates).

### DETAILED DESCRIPTION OF THE INVENTION

It has been determined herein that a targeted set combining genomic (e.g., TP53, RB1, CPLD, AR) and epigenomic (e.g., hypo- and hyper-methylation sites) alterations applied to ctDNA is advantageously useful for identifying patients with CRPC-NE. These CRPC-NE specific features were identified by studying whole exome sequencing and whole genome bisulfite sequencing of cell free DNA (cfDNA) and of matched metastatic tumor biopsies from patients with metastatic prostate adenocarcinoma and CRPC-NE. It was also found that there was significantly higher concordance between cfDNA and biopsy tissue genomic alterations in CRPC-NE patients compared to castration resistant adenocarcinoma, supporting greater intra-individual genomic consistency across metastases. Allele-specific copy number and serial sampling analyses allowed for the detection and tracking of clonal and subclonal tumor cell populations. In addition, cfDNA methylation was indicative of circulating tumor content fraction, reflective of methylation patterns observed in biopsy tissues, and was capable of detecting CRPC-NE-associated epigenetic changes (e.g., hypermethylation of ASXL3 and SPDEF; hypomethylation of INSM1 and CDH2).

A circulating biomarker for CRPC-NE provides a technical improvement and useful advantage for the avoidance of invasive biopsy for patients, which is the current standard for CRPC-NE diagnosis. Furthermore, since there is a spectrum of histologies even within CRPC-NE, and as patients progress from adenocarcinoma to CRPC-NE, with mixed adenocarcinoma-neuroendocrine morphologies and hybrid tumors with overlapping features sometimes observed, identifying molecular features further provides a technical improvement and useful advantage in information that is complementary to that of a tumor biopsy. Non-invasive detection of molecular features of CRPC-NE can therefore lead to a improved and refined sub-classification and detect features in patients at high risk for transformation even without CRPC-NE histology, paving the way for early intervention treatment strategies.

Accordingly, the present invention provides methods of diagnosing, prognosing, and monitoring CRPC-NE involving detecting the present or absence of the genomic and/or epigenomic alterations identified for CRPC-NE, and methods of treating CRPC-NE by modulating these genomic and/or epigenomic features.

### I. Definitions

The articles “a” and “an” are used herein to refer to one or to more than one (i.e. to at least one) of the grammatical object of the article. By way of example, “an element” means one element or more than one element.

The term “altered amount” or “altered level” refers to increased or decreased copy number (e.g., germline and/or somatic) of a biomarker nucleic acid, e.g., increased or decreased expression level in a cancer sample, as compared to the expression level or copy number of the biomarker nucleic acid in a control sample. The term “altered amount” of a biomarker also includes an increased or decreased protein level of a biomarker protein in a sample, e.g., a cancer sample, as compared to the corresponding protein level in a normal, control sample. Furthermore, an altered amount of a biomarker protein may be determined by detecting posttranslational modification such as methylation status of the marker, which may affect the expression or activity of the biomarker protein.

The amount of a biomarker in a subject is “significantly” higher or lower than the normal amount of the biomarker, if the amount of the biomarker is greater or less, respectively, than the normal level by an amount greater than the standard error of the assay employed to assess amount, and preferably at least 10%, 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the biomarker in the subject can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, inhibition, cytotoxicity, cell growth, and the like.

The term “altered level of expression” of a biomarker refers to an expression level or copy number of the biomarker in a test sample, e.g., a sample derived from a patient suffering from cancer, that is greater or less than the standard error of the assay employed to assess expression or copy number, and is preferably at least twice, and more preferably three, four, five or ten or more times the expression level or copy number of the biomarker in a control sample (e.g., sample from a healthy subjects not having the associated disease) and preferably, the average expression level or copy number of the biomarker in several control samples. The altered level of expression is greater or less than the standard error of the assay employed to assess expression or copy number, and is preferably at least 10%, 20%, 30%, 40%, 50%, 60%, 70%, 80%, 90%, 100%, 150%, 200%, 300%, 350%, 400%, 500%, 600%, 700%, 800%, 900%, 1000% or more times the expression level or copy number of the biomarker in a control sample (e.g., sample from a healthy subjects not having the associated disease) and preferably, the average expression level or copy number of the biomarker in several control samples. In some embodiments, the level of the biomarker refers to the level of the biomarker itself, the level of a modified biomarker (e.g., phosphorylated biomarker), or to the level of a biomarker relative to another measured variable, such as a control (e.g., phosphorylated biomarker relative to an unphosphorylated biomarker).

The term “altered activity” of a biomarker refers to an activity of the biomarker which is increased or decreased in a disease state, e.g., in a cancer sample, as compared to the activity of the biomarker in a normal, control sample. Altered activity of the biomarker may be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors.

The term “altered structure” of a biomarker refers to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations may be present in the coding or non-coding region of the biomarker nucleic acid.

Unless otherwise specified here within, the terms “antibody” and “antibodies” refers to antigen-binding portions adaptable to be expressed within cells as “intracellular antibodies.” (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publs. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

Antibodies may be polyclonal or monoclonal; xenogeneic, allogeneic, or syngeneic; or modified forms thereof (e.g. humanized, chimeric, etc.). Antibodies may also be fully human. Preferably, antibodies of the present invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized”, which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the present invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

The term “assigned score” refers to the numerical value designated for each of the biomarkers after being measured in a patient sample. The assigned score correlates to the absence, presence or inferred amount of the biomarker in the sample. The assigned score can be generated manually (e.g., by visual inspection) or with the aid of instrumentation for image acquisition and analysis. In certain embodiments, the assigned score is determined by a qualitative assessment, for example, detection of a fluorescent readout on a graded scale, or quantitative assessment. In one embodiment, an “aggregate score,” which refers to the combination of assigned scores from a plurality of measured biomarkers, is determined. In one embodiment the aggregate score is a summation of assigned scores. In another embodiment, combination of assigned scores involves performing mathematical operations on the assigned scores before combining them into an aggregate score. In certain, embodiments, the aggregate score is also referred to herein as the “predictive score.”

The term “biomarker” refers to a measurable entity of the present invention that has been determined to assess whether a subject is afflicted with castration-resistant neuroendocrine prosate cancer (CRPC-NE) or at risk for developing CRPC-NE. Biomarkers can include, without limitation, nucleic acids and proteins, including those shown in the Tables, the Examples, the Figures, and otherwise described herein. As described herein, any relevant characteristic of a biomarker can be used, such as the copy number, amount, activity, location, modification (e.g., phosphorylation), genomic alterations (e.g., deletion, gain, or mutation), epigenetic alterations (e.g., hypermethylation or hypomethylation) and the like.

A “blocking” antibody or an antibody “antagonist” is one which inhibits or reduces at least one biological activity of the antigen(s) it binds. In certain embodiments, the blocking antibodies or antagonist antibodies or fragments thereof described herein substantially or completely inhibit a given biological activity of the antigen(s).

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features. Unless otherwise stated, the terms include metaplasias.

Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

In certain embodiments, the cancer encompasses prostate cancer. Prostate cancer (Pca) is cancer that occurs in the prostate. Prostate cancer is one of the most common types of cancer in men. Usually prostate cancer grows slowly and is initially confined to the prostate gland, where it may not cause serious harm. However, other types are aggressive and can spread quickly.

Prostate cancer is typically diagnosed with a digital rectal exam and/or prostate specific antigen (PSA) screening. An elevated serum PSA level can indicate the presence of prostate cancer. PSA is used as a marker for prostate cancer because it is secreted only by prostate cells. When PSA or digital tests indicate a strong likelihood that cancer is present, a transrectal ultrasound (TRUS) is used to map the prostate and show any suspicious areas. Biopsies of various sectors of the prostate are used to determine if prostate cancer is present.

Treatment options depend on the stage of the cancer. Men with a 10-year life expectancy or less who have a low Gleason number and whose tumor has not spread beyond the prostate are often treated with watchful waiting (no treatment). Treatment options for more aggressive cancers include surgical treatments, such as radical prostatectomy (RP) in which the prostate is completely removed (with or without nerve sparing techniques), and radiation, applied through an external beam that directs the dose to the prostate from outside the body or via low-dose radioactive seeds that are implanted within the prostate to kill cancer cells locally. Anti-androgen hormone therapy may also be used, alone or in conjunction with surgery or radiation. Hormone therapy may use luteinizing hormone-releasing hormones (LH-RH) analogs, which block the pituitary from producing hormones that stimulate testosterone production. Patients may need to have injections of LH-RH analogs for the rest of their lives. While surgical and hormonal treatments are often effective for localized prostate cancer, advanced disease remains essentially incurable. Androgen ablation is the most common therapy for advanced prostate cancer, leading to massive apoptosis of androgen-dependent malignant cells and temporary tumor regression. However, the tumor may reemerge with a vengeance and can proliferate independent of androgen signals.

“Castrate-resistant prostate cancer (CRPC)” is defined by disease progression despite androgen-deprivation therapy (ADT) and may present as one or any combination of a continuous rise in serum levels of prostate-specific antigen (PSA), progression of pre-existing disease, or appearance of new metastases. Advanced prostate cancer has been known by a number of names over the years, including hormone-resistant prostate cancer (HRPC) and androgen-insensitive prostate cancer. Most recently, the terms “castrate-resistant” or “castration-recurrent” prostate cancer were introduced with the realization that intracrine and paracrine androgen production plays a significant role in the resistance of prostate cancer cells to testosterone-suppression therapy. In their second publication, the Prostate Cancer Working Group (PCWG2) defined CRPC as a continuum on the basis of whether metastases are detectable (clinically or by imaging) and whether serum testosterone is in the castrate range because of a surgical orchiectomy or medical therapy (Scher et al. (2008) J Clin Oncol. 26:1148-1159). The resulting clinical-states model can be used to classify patients. Within the rising PSA states (castrate and non-castrate), no detectable (measurable or non-measurable) disease has ever been found. Alternatively, in the clinical metastases states (castrate and non-castrate), disease has to have been detectable at some point in the past, regardless of whether it is currently detectable. Castrate-resistant prostate cancer presents a spectrum of disease ranging from rising PSA levels without metastases or symptoms and despite adt, to metastases and significant debilitation from cancer symptoms.

Prognosis is associated with several factors, including performance status, presence of bone pain, extent of disease on bone scan, and serum levels of alkaline phosphatase. Bone metastases occur in 90% of men with CRPC and can produce significant morbidity, including pain, pathologic fractures, spinal cord compression, and bone marrow failure. Paraneoplastic effects are also common, including anemia, weight loss, fatigue, hypercoagulability, and increased susceptibility to infection.

The mainstay of therapy for patients with metastatic spread, including castration-resistant prosate cancer, is hormonal therapy that targets the AR. For example, enzalutamid and abriraterone are potent AR-targeted therapies approved for the treatment of men with CRPC. Other treatment methods include, but are not limited to, other alternative hormone therapies, taxane chemotherapy (e.g., docetaxel, cabazitaxel), bone-targeting radiopharmaceuticals (e.g., radium-223) and immunotherapy (e.g., sipuleucel-T), etc., with the goals of prolonging survival, minimizing complications, and maintaining quality of life.

Castration-resistant prostate cancer is histologically characterized as prostate adenocarcinomas (CRPC-Adeno) and neuroendocrine prosate cancer (CRPC-NE). Histologically, NEPC is characterized by the presence of small, round, blue neuroendocrine carcinoma cells, which do not express androgen receptor or secrete prostate specific antigen (PSA), but usually express neuroendocrine markers such as chromogranin A, synaptophysin, and neuron-specific enolase.

Neuroendocrine prostate cancer (NEPC) is an aggressive subtype of prostate cancer that can arise de novo, but much more commonly arises after hormonal therapy for prostate adenocarcinoma (PCA) (PALMGREN et al., Semin Oncol, 34:22-9 (2007)). It is known that the amount of neuroendocrine differentiation increases with disease progression and correlates with patient exposure to long-term androgen deprivation therapy. NEPC reportedly differs histologically from PCA, and is characterized by the presence of small round blue neuroendocrine cells, which do not express androgen receptor (AR) or secrete prostate specific antigen (PSA), but usually express neureondocrine markers such as chromogranin A, synaptophysin, and neuron specific enolase (NSE)(Wang et al. (2008) Am Surg Pathol. 32:65-71). The prostate cancer specific TMPRSS2-ERG gene rearrangement (Tomlins et al. (2005) Science 310:644-648) has been reported in approximately 50% of NEPC (Lotan et al. (2011) Mod Pathol.), similar to the frequency in PCA (Mosquera et al. (2009) Clin Cancer Res. 15:4706-4711). This suggests clonal origin of NEPC from PCA and distinguishes NEPC from small carcinomas of other primary sites (Scheble et al. (2010) Mod Pathol. 23:1061-1067). The poor molecular characterization of NEPC accounts in part for the lack of disease specific therapeutics.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods of the present invention are not limited to use of a specific cut-point in comparing the level of expression product in the test sample to the control.

The “copy number” of a biomarker nucleic acid refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. For example, germline copy number changes include changes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes include changes at one or more genomic loci, wherein said one or more genomic loci are not accounted for by the number of copies in germline DNA of a control (e.g., copy number in germline DNA for the same subject as that from which the somatic DNA and corresponding copy number were determined).

The “normal” copy number (e.g., germline and/or somatic) of a biomarker nucleic acid or “normal” level of expression of a biomarker nucleic acid or protein is the activity/level of expression or copy number in a biological sample, e.g., a sample containing tissue, whole blood, serum, plasma, buccal scrape, saliva, cerebrospinal fluid, urine, stool, and bone marrow, from a subject, e.g., a human, not afflicted with cancer, or from a corresponding non-cancerous tissue in the same subject who has cancer.

As used herein, the term “costimulate” with reference to activated immune cells includes the ability of a costimulatory molecule to provide a second, non-activating receptor mediated signal (a “costimulatory signal”) that induces proliferation or effector function. For example, a costimulatory signal can result in cytokine secretion, e.g., in a T cell that has received a T cell-receptor-mediated signal. Immune cells that have received a cell-receptor mediated signal, e.g., via an activating receptor are referred to herein as “activated immune cells.”

The term “determining a suitable treatment regimen for the subject” is taken to mean the determination of a treatment regimen (i.e., a single therapy or a combination of different therapies that are used for the prevention and/or treatment of the cancer in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

The term “diagnosing cancer” includes the use of the methods, systems, and code of the present invention to determine the presence or absence of a cancer or subtype thereof in an individual. The term also includes methods, systems, and code for assessing the level of disease activity in an individual.

A molecule is “fixed” or “affixed” to a substrate if it is covalently or non-covalently associated with the substrate such that the substrate can be rinsed with a fluid (e.g. standard saline citrate, pH 7.4) without a substantial fraction of the molecule dissociating from the substrate.

The term “expression signature” or “signature” refers to a group of one or more coordinately expressed biomarkers related to a measured phenotype. For example, the genes, proteins, metabolites, and the like making up this signature may be expressed in a specific cell lineage, stage of differentiation, or during a particular biological response. The biomarkers can reflect biological aspects of the tumors in which they are expressed, such as the cell of origin of the cancer, the nature of the non-malignant cells in the biopsy, and the oncogenic mechanisms responsible for the cancer. Expression data and gene expression levels can be stored on computer readable media, e.g., the computer readable medium used in conjunction with a microarray or chip reading device. Such expression data can be manipulated to generate expression signatures.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

The term “immunotherapy” or “immunotherapies” refer to any treatment that uses certain parts of a subject's immune system to fight diseases such as cancer. The subject's own immune system is stimulated (or suppressed), with or without administration of one or more agent for that purpose. Immunotherapies that are designed to elicit or amplify an immune response are referred to as “activation immunotherapies.” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In some embodiments, the immunotherapy described herein comprises at least one of immunogenic chemotherapies. The term “immunogenic chemotherapy” refers to any chemotherapy that has been demonstrated to induce immunogenic cell death, a state that is detectable by the release of one or more damage-associated molecular pattern (DAMP) molecules, including, but not limited to, calreticulin, ATP and HMGB1 (Kroemer et al. (2013), Annu. Rev. Immunol., 31:51-72). Specific representative examples of consensus immunogenic chemotherapies include anthracyclines, such as doxorubicin and the platinum drug, oxaliplatin, 5′-fluorouracil, among others.

In some embodiments, immunotherapy comprises inhibitors of one or more immune checkpoints. The term “immune checkpoint” refers to a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR (see, for example, WO 2012/177624). The term further encompasses biologically active protein fragment, as well as nucleic acids encoding full-length immune checkpoint proteins and biologically active protein fragments thereof. In some embodiment, the term further encompasses any fragment according to homology descriptions provided herein. In one embodiment, the immune checkpoint is PD-1.

Immune checkpoints and their sequences are well-known in the art and representative embodiments are described below. For example, the term “PD-1” refers to a member of the immunoglobulin gene superfamily that functions as a coinhibitory receptor having PD-L1 and PD-L2 as known ligands. PD-1 was previously identified using a subtraction cloning based approach to select for genes upregulated during TCR-induced activated T cell death. PD-1 is a member of the CD28/CTLA-4 family of molecules based on its ability to bind to PD-L1. Like CTLA-4, PD-1 is rapidly induced on the surface of T-cells in response to anti-CD3 (Agata et al. 25 (1996) Int. Immunol. 8:765). In contrast to CTLA-4, however, PD-1 is also induced on the surface of B-cells (in response to anti-IgM). PD-1 is also expressed on a subset of thymocytes and myeloid cells (Agata et al. (1996) supra; Nishimura et al. (1996) Int. Immunol. 8:773).

“Anti-immune checkpoint therapy” refers to the use of agents that inhibit immune checkpoint nucleic acids and/or proteins. Inhibition of one or more immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint nucleic acids, or fragments thereof. Exemplary agents for upregulating an immune response include antibodies against one or more immune checkpoint proteins block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g. the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies, anti-PD-L1 antibodies, and/or anti-PD-L2 antibodies, either alone or in combination, are used to inhibit immune checkpoints. These embodiments are also applicable to specific therapy against particular immune checkpoints, such as the PD-1 pathway (e.g., anti-PD-1 pathway therapy, otherwise known as PD-1 pathway inhibitor therapy).

The term “TP53” refers to Tumor Protein P53, a tumor suppressor protein containing transcriptional activation, DNA binding, and oligomerization domains. The encoded protein responds to diverse cellular stresses to regulate expression of target genes, thereby inducing cell cycle arrest, apoptosis, senescence, DNA repair, or changes in metabolism. Mutations in this gene are associated with a variety of human cancers, including hereditary cancers such as Li-Fraumeni syndrome. TP53 mutations are universal across cancer types. The loss of a tumor suppressor is most often through large deleterious events, such as frameshift mutations, or premature stop codons. In TP53 however, many of the observed mutations in cancer are found to be single nucleotide missense variants. These variants are broadly distributed throughout the gene, but with the majority localizing in the DNA binding domain. There is no single hotspot in the DNA binding domain, but a majority of mutations occur in amino acid positions 175, 245, 248, 273, and 282 (NM 000546). While a large proportion of cancer genomics research is focused on somatic variants, TP53 is also of note in the germline. Germline TP53 mutations are the hallmark of Li-Fraumeni syndrome, and many (both germline and somatic) variants have been found to have a prognostic impact on patient outcomes. TP53 acts as a tumor suppressor in many tumor types by inducing growth arrest or apoptosis depending on the physiological circumstances and cell type. TP53 is involved in cell cycle regulation as a trans-activator that acts to negatively regulate cell division by controlling a set of genes required for this process. One of the activated genes is an inhibitor of cyclin-dependent kinases. Apoptosis induction seems to be mediated either by stimulation of BAX and FAS antigen expression, or by repression of Bcl-2 expression. In cooperation with mitochondrial PPIF, TP53 is involved in activating oxidative stress-induced necrosis, and the function is largely independent of transcription. TP53 induces the transcription of long intergenic non-coding RNA p21 (lincRNA-p21) and lincRNA-Mkln1. LincRNA-p21 participates in TP53-dependent transcriptional repression leading to apoptosis and seem to have to effect on cell-cycle regulation. TP53 is implicated in Notch signaling cross-over. TP53 prevents CDK7 kinase activity when associated to CAK complex in response to DNA damage, thus stopping cell cycle progression. Isoform 2 of TP53 enhances the transactivation activity of isoform 1 from some but not all TP53-inducible promoters. Isoform 4 of TP53 suppresses transactivation activity and impairs growth suppression mediated by isoform 1. Isoform 7 of TP53 inhibits isoform 1-mediated apoptosis. TP53 regulates the circadian clock by repressing CLOCK-ARNTL/BMAL1-mediated transcriptional activation of PER2 (Miki et al., (2013) Nat Commun 4:2444). In some embodiments, human TP53 protein has 393 amino acids and a molecular mass of 43653 Da. The known binding partners of TP53 include, e.g., AXIN1, ING4, YWHAZ, HIPK1, HIPK2, WWOX, GRKS, ANKRD2, RFFL, RNF 34, and TP53INP1.

The term “TP53” is intended to include fragments, variants (e.g., allelic variants), and derivatives thereof. Representative human TP53 cDNA and human TP53 protein sequences are well-known in the art and are publicly available from the National Center for Biotechnology Information (NCBI). For example, at least 12 different human TP53 isoforms are known. Human TP53 isoform a (NP_000537.3, NP_001119584.1) is encodable by the transcript variant 1 (NM_000546.5) and the transcript variant 2 (NM_001126112.2). Human TP53 isoform b (NP_001119586.1) is encodable by the transcript variant 3 (NM_001126114.2). Human TP53 isoform c (NP_001119585.1) is encodable by the transcript variant 4 (NM_001126113.2). Human TP53 isoform d (NP_001119587.1) is encodable by the transcript variant 5 (NM_001126115.1). Human TP53 isoform e (NP_001119588.1) is encodable by the transcript variant 6 (NM_001126116.1). Human TP53 isoform f (NP_001119589.1) is encodable by the transcript variant 7 (NM_001126117.1). Human TP53 isoform g (NP_001119590.1, NP_001263689.1, and NP_001263690.1) is encodable by the transcript variant 8 (NM_001126118.1), the transcript variant 1 (NM_001276760.1), and the transcript variant 2 (NM_001276761.1). Human TP53 isoform h (NP_001263624.1) is encodable by the transcript variant 4 (NM_001276695.1). Human TP53 isoform i (NP_001263625.1) is encodable by the transcript variant 3 (NM_001276696.1). Human TP53 isoform j (NP_001263626.1) is encodable by the transcript variant 5 (NM_001276697.1). Human TP53 isoform k (NP_001263627.1) is encodable by the transcript variant 6 (NM_001276698.1). Human TP53 isoform 1 (NP_001263628.1) is encodable by the transcript variant 7 (NM_001276699.1). Nucleic acid and polypeptide sequences of TP53 orthologs in organisms other than humans are well known and include, for example, chimpanzee TP53 (XM 001172077.5 and XP 001172077.2, and XM 016931470.2 and) CP 016786959.2), monkey TP53 (NM_001047151.2 and NP_001040616.1), dog TP53 (NM_001003210.1 and NP_001003210.1), cattle TP53 (NM_174201.2 and NP_776626.1), mouse TP53 (NM_001127233.1 and NP_001120705.1, and NM_011640.3 and NP_035770.2), rat TP53 (NM_030989.3 and NP_112251.2), tropical clawed frog TP53 (NM_001001903.1 and NP_001001903.1), and zebrafish TP53 (NM_001271820.1 and NP_001258749.1, NM_001328587.1 and NP_001315516.1, NM_001328588.1 and NP_001315517.1, and NM_131327.2 and NP_571402.1). Representative sequences of TP53 orthologs are presented below in Table 1A.

Anti-TP53 antibodies suitable for detecting TP53 protein are well-known in the art and include, for example, antibodies TA502925 and CF502924 (Origene), antibodies NB200-103 and NB200-171 (Novus Biologicals, Littleton, Colo.), antibodies ab26 and ab1101 (AbCam, Cambridge, Mass.), antibody 700439 (ThermoFisher Scientific), antibody 33-856 (ProSci), etc. In addition, reagents are well-known for detecting TP53. Multiple clinical tests of TP53 are available in NIH Genetic Testing Registry (GTR®) (e.g., GTR Test ID: GTR000517320.2, offered by Fulgent Clinical Diagnostics Lab (Temple City, Calif.)). Moreover, multiple siRNA, shRNA, CRISPR constructs for reducing TP53 expression can be found in the commercial product lists of the above-referenced companies, such as siRNA products #sc-29435 and sc-44218, and CRISPR product # sc-416469 from Santa Cruz Biotechnology, RNAi products SR322075 and TL320558V, and CRISPR product KN200003 (Origene), and multiple CRISPR products from GenScript (Piscataway, N.J.). Chemical inhibitors of TP53 are also available, including, e.g., Cyclic Pifithrin-a hydrobromide, RITA (TOCRIS, Minn.). It is to be noted that the term can further be used to refer to any combination of features described herein regarding TP53 molecules. For example, any combination of sequence composition, percentage identify, sequence length, domain structure, functional activity, etc. can be used to describe a TP53 molecule encompassed by the present invention.

The term “RB1”, also known as RB transcriptional corepressor 1, is a negative regulator of the cell cycle and was the first tumor suppressor gene found. The encoded protein also stabilizes constitutive heterochromatin to maintain the overall chromatin structure. The active, hypophosphorylated form of the protein binds transcription factor E2F1. Defects in this gene are a cause of childhood cancer retinoblastoma (RB), bladder cancer, and osteogenic sarcoma. RB1 is a key regulator of entry into cell division that acts as a tumor suppressor. It promotes G0-G1 transition when phosphorylated by CDK3/cyclin-C. RB1 acts as a transcription repressor of E2F1 target genes. The underphosphorylated, active form of RB1 interacts with E2F1 and represses its transcription activity, leading to cell cycle arrest. RB1 is also directly involved in heterochromatin formation by maintaining overall chromatin structure and, in particular, that of constitutive heterochromatin by stabilizing histone methylation. RB1 recruits and targets histone methyltransferases SUV39H1, KMT5B and KMT5C, leading to epigenetic transcriptional repression. It controls histone H4 ‘Lys-20’ trimethylation. RB1 inhibits the intrinsic kinase activity of TAF1. It mediates transcriptional repression by SMARCA4/BRG1 by recruiting a histone deacetylase (HDAC) complex to the c-FOS promoter. In resting neurons, transcription of the c-FOS promoter is inhibited by BRG1-dependent recruitment of a phospho-RB1-HDAC1 repressor complex. Upon calcium influx, RB1 is dephosphorylated by calcineurin, which leads to release of the repressor complex. In case of viral infections, RB1 interactions with SV40 large T antigen, HPV E7 protein or adenovirus E1A protein and induces the disassembly of RB1-E2F1 complex thereby disrupting RB1's activity.

The term “RB1” is intended to include fragments, variants (e.g., allelic variants), and derivatives thereof. Representative human RB1 cDNA and human RB1 protein sequences are well-known in the art and are publicly available from the National Center for Biotechnology Information (NCBI). For example, at least one different human RB1 isoforms are known. Human RB1 protein (NP_000312.2) is encodable by the transcript (NM_000321.2). Nucleic acid and polypeptide sequences of RB1 orthologs in organisms other than humans are well known and include, for example, chimpanzee RB1 (XM_509777.4 and XP 509777.3, and XM_016925689.1 and XP 016781178.1), dog RB1 (XM_022408288.1 and XP 022263996.1), cattle RB1 (NM_001076907.1 and NP_001070375.1), mouse RB1 (NM_009029.3 and NP_033055.2), rat RB1 (NM_017045.1 and NP_058741.1), chicken RB1 (NM_204419.1 and NP_989750.1), tropical clawed frog RB1 (NM_001282525.1 and NP_001269454.1), and zebrafish RB1 (NM_001077780.1 and NP_001071248.1). Representative sequences of RB1 orthologs are presented below in Table 1A.

The term “CYLD,” also known as “CYLD lysine 63 deubiquitinase,” refers to a cytoplasmic protein with three cytoskeletal-associated protein-glycine-conserved (CAP-GLY) domains that functions as a deubiquitinating enzyme. Mutations in this gene have been associated with cylindromatosis, multiple familial trichoepithelioma, and Brooke-Spiegler syndrome. CYLD is a deubiquitinase that specifically cleaves Lys-63- and linear Met-1-linked polyubiquitin chains and is involved in NF-kappa-B activation and TNF-alpha-induced necroptosis. It plays an important role in the regulation of pathways leading to NF-kappa-B activation. CYLD contributes to the regulation of cell survival, proliferation and differentiation via its effects on NF-kappa-B activation. It is also a negative regulator of Wnt signaling. CYLD inhibits HDAC6 and thereby promotes acetylation of alpha-tubulin and stabilization of microtubules. It plays a role in the regulation of microtubule dynamics, and thereby contributes to the regulation of cell proliferation, cell polarization, cell migration, and angiogenesis. CYLD is required for normal cell cycle progress and normal cytokinesis. It inhibits nuclear translocation of NF-kappa-B. CYLD plays a role in the regulation of inflammation and the innate immune response, via its effects on NF-kappa-B activation. It is dispensable for the maturation of intrathymic natural killer cells, but required for the continued survival of immature natural killer cells. In addition, CYLD negatively regulates TNFRSF11A signaling and osteoclastogenesis, and is involved in the regulation of ciliogenesis, allowing ciliary basal bodies to migrate and dock to the plasma membrane; this process does not depend on NF-kappa-B activation. CYLD is able to remove linear (Met-1-linked) polyubiquitin chains and regulates innate immunity and TNF-alpha-induced necroptosis. It is recruited to the LUBAC complex via interaction with SPATA2 and restricts linear polyubiquitin formation on target proteins. Furthermore, CYLD regulates innate immunity by restricting linear polyubiquitin formation on RIPK2 in response to NOD2 stimulation. CYLD is involved in TNF-alpha-induced necroptosis by removing linear (Met-1-linked) polyubiquitin chains from RIPK1, thereby regulating the kinase activity of RIPK1. CYLD removes Lys linked polyubiquitin chain of MAP3K7, which inhibits phosphorylation and blocks downstream activation of the JNK-p38 kinase cascades.

The term “CYLD” is intended to include fragments, variants (e.g., allelic variants), and derivatives thereof. Representative human CYLD cDNA and human CYLD protein sequences are well-known in the art and are publicly available from the National Center for Biotechnology Information (NCBI). For example, at least 3 different human CYLD isoforms are known. Human CYLD isoform 1 (NP_056062.1) is encodable by the transcript variant 1 (NM_015247.2). Human CYLD isoform 2 (NP_001035814.1 and NP_001035877.1) is encodable by the transcript variant 2 (NM_001042355.2) and the transcript variant 3 (NM_001042412.2). Nucleic acid and polypeptide sequences of CYLD orthologs in organisms other than humans are well known and include, for example, chimpanzee CYLD (XM_016929820.2 and XP 016785309.1, XM_016929821.2 and) CP_016785310.1, XM_016929822.2 and XP 016785311.1, XM_016929818.2 and XP_016785307.1, XM_009430770.3 and XP_009429045.1, and XM_016929819.2 and XP_016785308.1), monkey CYLD (XM_015126108.2 and (CP_014981594.1, XM_015126112.2 and XP_014981598.1, XM_015126111.2 and XP_014981597.1, XM_015126109.2 and XP_014981595.1, and XM_015126113.2 and XP_014981599.1), dog CYLD (XM_005617567.3 and XP_005617624.1, XM_005617569.3 and XP_005617626.1, XM_005617568.3 and XP_005617625.1, and XM_849343.5 and XP_854436.1), cattle CYLD (NM_001046417.1 and NP_001039882.1), mouse CYLD (NM_001128170.2 and NP_001121642.1, NM_001128171.2 and NP_001121643.1, NM_001276279.1 and NP_001263208.1, and NM_173369.3 and NP_775545.1), rat CYLD (NM_001017380.1 and NP_001017380.1), and chicken CYLD (XM_015292398.2 and XP_015147884.2). Representative sequences of CYLD orthologs are presented below in Table 1A.

“Androgen receptor” (“AR”) refers to the steroid receptor that is located in the cytoplasm and translocated to the nucleus upon stimulation with androgen or an androgen analogue. AR is a soluble protein that functions as an intracellular transcription factor. The function of AR is regulated by the binding of androgen, which initiates successive conformational changes in the receptor and thus affects the interaction between the receptor and the protein and the interaction between the receptor and DNA. AR mediates the induction of various biological effects through interaction with endogenous androgens. Endogenous androgens include steroids such as testosterone and dihydrotinone. In many tissues, testosterone is converted to dihydrocinone by enzyme 5α-reductase.

AR is mainly expressed in androgen target tissues such as prostate, skeletal muscle, liver and central nervous system (CNS), while the highest observed in the prostate, adrenal gland and parasitoid. AR can be activated by binding to endogenous androgens, including testosterone and 5α-dihydrosterolone (5α-DHT).

The Xq11-12 androgen receptor (AR) is a 110 kD nuclear receptor that mediates transcription of a target gene that regulates differentiation and growth of prostate epithelial cells upon activation by androgen. Similar to other steroid receptors, unbound AR is primarily located in the cytoplasm and is associated with complexes of heat shock proteins (HSPs) by interacting with ligand binding domains. After binding to the agonist, the AR undergoes a series of conformational changes: the heat shock protein is isolated from the AR, and the transformed AR is dimerized, phosphorylated, and mediated by the nuclear localization signal to the nucleus. The transfer site receptor then binds to the androgen response unit (ARE), which is characterized by a hexanucleotide half-site consensus sequence 5′-TGTTCT-3′ (by three random nucleotides) Interval) and located in the promoter or enhancer region of the AR target gene. The convening of other transcriptional co-regulators (including co-activators and co-repressors) and transcriptional machinery further ensures transcriptional activation of AR-regulated gene expression. All of these processes are initiated by ligand-induced conformational changes in the ligand binding domain.

The effects of androgen and androgen receptors are known to be associated with diseases or conditions such as prostate cancer, breast cancer, androgen-dependent hirsutism, male alopecia, uterine fibroids, leiomyoma, endometrial cancer or endometrium. The signaling of AR is critical for the development and maintenance of the male reproductive organs, including the prostate, because male genetically inactive AR mutants and genetically engineered AR-deficient mice do not develop prostate or prostate cancer. The dependence of prostate cells on AR signal transmission persists even after malignant tumor formation. Androgen depletion (such as the use of GnRH agonists) remains the primary method of treating prostate cancer. However, androgen removal often only effective for a limited period of time, although the amount of circulating androgen is very low, prostate cancer will gradually resume growth. Castration-resistant prostate cancer (CRPC) is a fatal phenotype and almost all patients die of prostate cancer.

The term “AR” is intended to include fragments, variants (e.g., allelic variants), and derivatives thereof. Representative human AR cDNA and human AR protein sequences are well-known in the art and are publicly available from the National Center for Biotechnology Information (NCBI). For example, at least 5 different human AR isoforms are known. Human AR isoform 1 (NP_000035.2) is encodable by the transcript variant 1 (NM_000044.6). Human AR isoform 2 (NP_001011645.1) is encodable by the transcript variant 2 (NM_001011645.3). Human AR isoform 3 (NP_001334990.1) is encodable by the transcript variant 3 (NM_001348061.1). Human AR isoform 4 (NP_001334992.1) is encodable by the transcript variant 4 (NM_001348063.1). Human AR isoform 5 (NP_001334993.1) is encodable by the transcript variant 5 (NM_001348064.1). Nucleic acid and polypeptide sequences of AR orthologs in organisms other than humans are well known and include, for example, monkey AR (NM_001032911.1 and NP_001028083.1), dog AR (NM_001003053.1 and NP_001003053.1), cattle AR (NM_001244127.1 and NP_001231056.1), mouse AR (NM_013476.4 and NP_038504.1), rat AR (NM_012502.1 and NP_036634.1), and chicken AR (NM_001040090.1 and NP_001035179.1). Representative sequences of AR orthologs are presented below in Table 1B.

The term “androgen receptor-directed therapy” to any therapy used for the treatment of prostate cancer by targeting androgen receptor (AR)-signaling in the subject. The therapy may act through inhibition of androgen synthesis or through AR targeting directly. Exemplary androgen receptor (AR)-directed therapies include, but are not limited to abiraterone or enzalutamide.

The term “NEPC score” refers to a score that is calculated based on the combination of specific genomic and/or epigenomic alterations of biomarkers described herein. In one embodiment, this score may be used to diagnose whether a subject is afflicted with castration-resistant neuroendocrine prosate cancer (CRPC-NE) or at risk for developing CRPC-NE. In another embodiment, it may be used to monitor the progression of CRPC or predict the efficacy of an agent for treating CRPC-NE in a subject. In still another embodiment, it may be used to predict the likelihood of response to an AR-targeted therapy in a subject afflicted with CRPC. The NEPC score may be calculated according to the methods described in example 1 of the present disclosure.

The term “epigenomic alteration” refers to any epigenetic modification on the genetic material (e.g., genomic DNA) of a cell or a subject. In certain embodiment, the epigenomic alteration is DNA methylation. DNA methylation is a chemical modification of DNA performed by enzymes called methyltransferases, in which a methyl group (m) is added to certain cytosines (C) of DNA. This non-mutational (epigenetic) process (mC) is a critical factor in gene expression regulation. DNA methylation plays an important role in determining gene expression. By turning genes off that are not needed, DNA methylation is an essential control mechanism for the normal development and functioning of organisms. Alternatively, abnormal DNA methylation is one of the mechanisms underlying the changes observed with the development of many cancers.

The best-defined epigenetic alteration of cancer genes involves DNA methylation of clustered CpG dinucleotides, or CpG islands, in promoter regions associated with the transcriptional inactivation of the affected genes. CpG islands are short sequences rich in the CpG dinucleotide, and can be found in the 5′ region of about half of all human genes. Methylation of cytosine within 5′ CGIs is associated with loss of gene expression and has been seen in a number of physiological conditions, including X chromosome inactivation and genomic imprinting. Aberrant methylation of CpG islands has been detected in genetic diseases such as the fragile-X syndrome, in aging cells and in neoplasia. About half of the tumor suppressor genes which have been shown to be mutated in the germline of patients with familial cancer syndromes have also been shown to be aberrantly methylated in some proportion of sporadic cancers, including Rb, VHL, p16, hMLH1, and BRCA1. Methylation of tumor suppressor genes in cancer is usually associated with (1) lack of gene transcription and (2) absence of coding region mutation. Thus CpG island methylation can serve as an alternative mechanism of gene inactivation in cancer.

The term “hypermethylation” refers to an increase in the epigenetic methylation of cytosine and adenosine residues in DNA from a sample compared to a control. The term “hypomethylation” refers to a decrease in the epigenetic methylation of cytosine and adenosine residues in DNA from a sample compared to a control. In certain embodiments, the control is a site specific tissue-based threshold that discriminates between castration resistant prostate adenocarcinoma (CRPC-Adeno) and CRPC-NE tissue samples. Such thresholds can be determined using methods described in example 1. In some embodiment, the control is the site specific tissue-based methylation level determined in CRPC-Adeno samples.

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

The term “immunotherapeutic agent” can include any molecule, peptide, antibody or other agent which can stimulate a host immune system to generate an immune response to a tumor or cancer in the subject. Various immunotherapeutic agents are useful in the compositions and methods described herein.

The term “inhibit” includes the decrease, limitation, or blockage, of, for example a particular action, function, or interaction. In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

The term “interaction”, when referring to an interaction between two molecules, refers to the physical contact (e.g., binding) of the molecules with one another. Generally, such an interaction results in an activity (which produces a biological effect) of one or both of said molecules.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), more preferably less than about 20% of non-biomarker protein, still more preferably less than about 10% of non-biomarker protein, and most preferably less than about 5% non-biomarker protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

As used herein, the term “isotype” refers to the antibody class (e.g., IgM, IgG1, IgG2C, and the like) that is encoded by heavy chain constant region genes.

As used herein, the term “K_{D}” is intended to refer to the dissociation equilibrium constant of a particular antibody-antigen interaction. The binding affinity of antibodies of the disclosed invention may be measured or determined by standard antibody-antigen assays, for example, competitive assays, saturation assays, or standard immunoassays such as ELISA or RIA.

A “kit” is any manufacture (e.g. a package or container) comprising at least one reagent, e.g. a probe or small molecule, for specifically detecting and/or affecting the expression of a marker of the present invention. The kit may be promoted, distributed, or sold as a unit for performing the methods of the present invention. The kit may comprise one or more reagents necessary to express a composition useful in the methods of the present invention. In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit can be included.

The term “neoadjuvant therapy” refers to a treatment given before the primary treatment. Examples of neoadjuvant therapy can include chemotherapy, radiation therapy, and hormone therapy. For example, in treating breast cancer, neoadjuvant therapy can allows patients with large breast cancer to undergo breast-conserving surgery.

The “normal” level of expression of a biomarker is the level of expression of the biomarker in cells of a subject, e.g., a human patient, not afflicted with a cancer. An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

The term “pre-determined” biomarker genomic and/or epigenomic alteration(s) may be a biomarker genomic and/or epigenomic alteration(s) used to, by way of example only, evaluate a subject that may be selected for a particular treatment, evaluate a response to a treatment, and/or evaluate the disease state. A pre-determined biomarker genomic and/or epigenomic alteration(s) may be determined in populations of patients with or without cancer. The pre-determined biomarker genomic and/or epigenomic alteration(s) can be a single number, equally applicable to every patient, or the pre-determined biomarker genomic and/or epigenomic alteration(s) can vary according to specific subpopulations of patients. Age, weight, height, and other factors of a subject may affect the pre-determined biomarker genomic and/or epigenomic alteration(s) of the individual. Furthermore, the pre-determined biomarker amount and/or activity can be determined for each subject individually. In one embodiment, the amounts determined and/or compared in a method described herein are based on absolute measurements. In another embodiment, the amounts determined and/or compared in a method described herein are based on relative measurements, such as ratios (e.g., serum biomarker normalized to the expression of housekeeping or otherwise generally constant biomarker). The pre-determined biomarker genomic and/or epigenomic alteration(s) can be any suitable standard. For example, the pre-determined biomarker genomic and/or epigenomic alteration(s) can be obtained from the same or a different human for whom a patient selection is being assessed. In one embodiment, the pre-determined biomarker genomic and/or epigenomic alteration(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

The term “predictive” includes the use of a biomarker nucleic acid and/or protein status, e.g., over- or under-activity, emergence, expression, growth, remission, recurrence or resistance of tumors before, during or after therapy, for determining the likelihood of response of a cancer to an anti-cancer therapy. Such predictive use of the biomarker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression of a biomarker nucleic acid (e.g., by ISH, Northern Blot, or qPCR), increased or decreased biomarker protein (e.g., by IHC), or increased or decreased activity, e.g., in more than about 5%, 6%, 7%, 8%, 9%, 10%, 11%, 12%, 13%, 14%, 15%, 20%, 25%, 30%, 40%, 50%, 60%, 70%, 80%, 90%, 95%, 100%, or more of assayed human cancers types or cancer samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its absolute or relatively modulated presence or absence in clinical subset of patients with cancer.

The term “pre-malignant lesions” as described herein refers to a lesion that, while not cancerous, has potential for becoming cancerous. It also includes the term “pre-malignant disorders” or “potentially malignant disorders.” In particular this refers to a benign, morphologically and/or histologically altered tissue that has a greater than normal risk of malignant transformation, and a disease or a patient's habit that does not necessarily alter the clinical appearance of local tissue but is associated with a greater than normal risk of precancerous lesion or cancer development in that tissue (leukoplakia, erythroplakia, erytroleukoplakia lichen planus (lichenoid reaction) and any lesion or an area which histological examination showed atypia of cells or dysplasia. In one embodiment, a metaplasia is a pre-malignant lesion.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “probe” refers to any molecule which is capable of selectively binding to a specifically intended target molecule, for example, a nucleotide transcript or protein encoded by or corresponding to a biomarker nucleic acid. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

The term “prognosis” includes a prediction of the probable course and outcome of cancer or the likelihood of recovery from the disease. In some embodiments, the use of statistical algorithms provides a prognosis of cancer in an individual. For example, the prognosis can be surgery, development of a clinical subtype of cancer (e.g., solid tumors, such as esophageal cancer and gastric cancer), development of one or more clinical factors, or recovery from the disease.

The term “response to an anti-cancer therapy” relates to any response of the hyperproliferative disorder (e.g., cancer) to an anti-cancer agent, preferably to a change in tumor mass and/or volume after initiation of neoadjuvant or adjuvant therapy. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following cancer therapy for which biomarker measurement values are known. In certain embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using well-known methods in the art, such as those described in the Examples section.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 25% or more, for example, 30%, 40%, 50%, 60%, 70%, 80%, or more, to 2-fold, 3-fold, 4-fold, 5-fold, 10-fold, 15-fold, 20-fold or more. The reduction in response can be measured by comparing with the same cancer sample or mammal before the resistance is acquired, or by comparing with a different cancer sample or a mammal that is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The terms “response” or “responsiveness” refers to an anti-cancer response, e.g. in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene of the present invention, or a fragment thereof, short interfering RNA (siRNA), and small molecules which interfere with or inhibit expression of a target biomarker nucleic acid by RNA interference (RNAi).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn and Cullen (2002) J. Virol. 76:9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease may be of at least 30%, 40%, 50%, 60%, 70%, 80%, 90%, 95% or 99% or more as compared to the expression of a target biomarker nucleic acid or the activity or level of the protein encoded by a target biomarker nucleic acid which has not been targeted by an RNA interfering agent.

The term “sample” used for detecting or determining the presence or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method of the present invention further comprises obtaining the sample from the individual prior to detecting or determining the presence or level of at least one marker in the sample.

The term “sensitize” means to alter cancer cells or tumor cells in a way that allows for more effective treatment of the associated cancer with a cancer therapy (e.g., anti-immune checkpoint, chemotherapeutic, and/or radiation therapy). In some embodiments, normal cells are not affected to an extent that causes the normal cells to be unduly injured by the therapies. An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42: 2159-2164), cell death assays (Weisenthal L M, Shoemaker R H, Marsden J A, Dill P L, Baker J A, Moran E M, Cancer Res 1984; 94: 161-173; Weisenthal L M, Lippman M E, Cancer Treat Rep 1985; 69: 615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993: 415-432; Weisenthal L M, Contrib Gynecol Obstet 1994; 19: 82-90). The sensitivity or resistance may also be measured in animal by measuring the tumor size reduction over a period of time, for example, 6 month for human and 4-6 weeks for mouse. A composition or a method sensitizes response to a therapeutic treatment if the increase in treatment sensitivity or the reduction in resistance is 25% or more, for example, 30%, 40%, 50%, 60%, 70%, 80%, or more, to 2-fold, 3-fold, 4-fold, 5-fold, 10-fold, 15-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

“Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated by reference herein).

RNA interfering agents, e.g., siRNA molecules, may be administered to a patient having or at risk for having cancer, to inhibit expression of a biomarker gene which is overexpressed in cancer and thereby treat, prevent, or inhibit cancer in the subject.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

The term “specific binding” refers to antibody binding to a predetermined antigen. Typically, the antibody binds with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” Selective binding is a relative term referring to the ability of an antibody to discriminate the binding of one antigen over another.

The term “subject” refers to any healthy animal, mammal or human, or any animal, mammal or human afflicted with a cancer, e.g., brain, lung, ovarian, pancreatic, liver, breast, prostate, and/or colorectal cancers, melanoma, multiple myeloma, and the like. The term “subject” is interchangeable with “patient.”

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

The term “synergistic effect” refers to the combined effect of two or more anti-cancer agents (e.g., modulator of genomic and/or epigenomic alterations of one or more biomarkers listed in Tables 1A-D/immunotherapy combination therapy) can be greater than the sum of the separate effects of the anti-cancer agents/therapies alone.

The term “T cell” includes CD4⁺ T cells and CD8⁺ T cells. The term T cell also includes both T helper 1 type T cells and T helper 2 type T cells. The term “antigen presenting cell” includes professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

The terms “therapeutically-effective amount” and “effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, Similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy can be achieved.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g. splicing), if any, of the RNA transcript, and reverse transcription of the RNA transcript.

As used herein, the term “unresponsiveness” includes refractivity of cancer cells to therapy or refractivity of therapeutic cells, such as immune cells, to stimulation, e.g., stimulation via an activating receptor or a cytokine. Unresponsiveness can occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. (1992) Science 257:1134).

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, | GAC, GAT |
| D) | |
| Cysteine (Cys, C) | TGC, TGT |
| Glutamic acid (Glu, | GAA, GAG |
| E) | |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, | TTC, TTT |
| F) | |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal | TAA, TAG, TGA |
| (end) | |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for the loci and biomarkers of the present invention (e.g., biomarkers listed in Tables 1A-1D) are well-known in the art and readily available on publicly available databases, such as the National Center for Biotechnology Information (NCBI).

TABLE 1A
TP53
RB1
CYLD

SEQ ID NO: 1 Human TP53 Isoform a Amino Acid Sequence (NP_000537.3,
NP_001119584.1)

| | |
|---|---|
| 1 | meepqsdpsv epplsqetfs dlwkllpenn vlsplpsqam ddlmlspddi eqwftedpgp |
| 61 | deaprmpeaa ppvapapaap tpaapapaps wplsssvpsq ktyqgsygfr lgflhsgtak |
| 121 | svtctyspal nkmfcqlakt cpvqlwvdst pppgtrvram aiykqsqhmt evvrrcphhe |
| 181 | rcsdsdglap pqhlirvegn lrveylddrn tfrhsvvvpy eppevgsdct tihynymcns |
| 241 | scmggmnrrp iltiitleds sgnllgrnsf evrvcacpgr drrteeenlr kkgephhelp |
| 301 | pgstkralpn ntssspqpkk kpldgeyftl qirgrerfem frelnealel kdaqagkepg |
| 361 | gsrahsshlk skkgqstsrh kklmfktegp dsd |

SEQ ID NO: 2 Human TP53 transcript variant 1 cDNA sequence (NM_000546.5;
CDS: 203-1384)

| | |
|---|---|
| 1 | gatgggattg gggttttccc ctcccatgtg ctcaagactg gcgctaaaag ttttgagctt |
| 61 | ctcaaaagtc tagagccacc gtccagggag caggtagctg ctgggctccg gggacacttt |
| 121 | gcgttcgggc tgggagcgtg ctttccacga cggtgacacg cttccctgga ttggcagcca |
| 181 | gactgccttc cgggtcactg ccatggagga gccgcagtca gatcctagcg tcgagccccc |
| 241 | tctgagtcag gaaacatttt cagacctatg gaaactactt cctgaaaaca acgttctgtc |
| 301 | ccccttgccg tcccaagcaa tggatgattt gatgctgtcc ccggacgata ttgaacaatg |
| 361 | gttcactgaa gacccaggtc cagatgaagc tcccagaatg ccagaggctg ctccccccgt |
| 421 | ggcccctgca ccagcagctc ctacaccggc ggcccctgca ccagccccct cctggcccct |
| 481 | gtcatcttct gtcccttccc agaaaaccta ccagggcagc tacggtttcc gtctgggctt |
| 541 | cttgcattct gggacagcca agtctgtgac ttgcacgtac tcccctgccc tcaacaagat |
| 601 | gttttgccaa ctggccaaga cctgccctgt gcagctgtgg gttgattcca cacccccgcc |
| 661 | cggcacccgc gtccgcgcca tggccatcta caagcagtca cagcacatga cggaggttgt |
| 721 | gaggcgctgc ccccaccatg agcgctgctc agatagcgat ggtctggccc ctcctcagca |
| 781 | tcttatccga gtggaaggaa atttgcgtgt ggagtatttg gatgacagaa acacttttcg |
| 841 | acatagtgtg gtggtgccct atgagccgcc tgaggttggc tctgactgta ccaccatcca |
| 901 | ctacaactac atgtgtaaca gttcctgcat gggcggcatg aaccggaggc ccatcctcac |
| 961 | catcatcaca ctggaagact ccagtggtaa tctactggga cggaacagct ttgaggtgcg |
| 1021 | tgtttgtgcc tgtcctggga gagaccggcg cacagaggaa gagaatctcc gcaagaaagg |
| 1081 | ggagcctcac cacgagctgc ccccagggag cactaagcga gcactgccca acaacaccag |
| 1141 | ctcctctccc cagccaaaga agaaaccact ggatggagaa tatttcaccc ttcagatccg |
| 1201 | tgggcgtgag cgcttcgaga tgttccgaga gctgaatgag gccttggaac tcaaggatgc |
| 1261 | ccaggctggg aaggagccag gggggagcag ggctcactcc agccacctga agtccaaaaa |
| 1321 | gggtcagtct acctcccgcc ataaaaaact catgttcaag acagaagggc ctgactcaga |
| 1381 | ctgacattct ccacttcttg ttccccactg acagcctccc acccccatct ctccctcccc |
| 1441 | tgccattttg ggttttgggt ctttgaaccc ttgcttgcaa taggtgtgcg tcagaagcac |
| 1501 | ccaggacttc catttgcttt gtcccggggc tccactgaac aagttggcct gcactggtgt |
| 1561 | tttgttgtgg ggaggaggat ggggagtagg acataccagc ttagatttta aggtttttac |
| 1621 | tgtgagggat gtttgggaga tgtaagaaat gttcttgcag ttaagggtta gtttacaatc |
| 1681 | agccacattc taggtagggg cccacttcac cgtactaacc agggaagctg tccctcactg |
| 1741 | ttgaattttc tctaacttca aggcccatat ctgtgaaatg ctggcatttg cacctacctc |
| 1801 | acagagtgca ttgtgagggt taatgaaata atgtacatct ggccttgaaa ccacctttta |
| 1861 | ttacatgggg tctagaactt gacccccttg agggtgcttg ttccctctcc ctgttggtcg |
| 1921 | gtgggttggt agtttctaca gttgggcagc tggttaggta gagggagttg tcaagtctct |
| 1981 | gctggcccag ccaaaccctg tctgacaacc tcttggtgaa ccttagtacc taaaaggaaa |
| 2041 | tctcacccca tcccacaccc tggaggattt catctcttgt atatgatgat ctggatccac |
| 2101 | caagacttgt tttatgctca gggtcaattt cttttttctt tttttttttt ttttttcttt |
| 2161 | ttctttgaga ctgggtctcg ctttgttgcc caggctggag tggagtggcg tgatcttggc |
| 2221 | ttactgcagc ctttgcctcc ccggctcgag cagtcctgcc tcagcctccg gagtagctgg |
| 2281 | gaccacaggt tcatgccacc atggccagcc aacttttgca tgttttgtag agatggggtc |
| 2341 | tcacagtgtt gcccaggctg gtctcaaact cctgggctca ggcgatccac ctgtctcagc |
| 2401 | ctcccagagt gctgggatta caattgtgag ccaccacgtc cagctggaag ggtcaacatc |
| 2461 | ttttacattc tgcaagcaca tctgcatttt caccccaccc ttcccctcct tctccctttt |
| 2521 | tatatcccat ttttatatcg atctcttatt ttacaataaa actttgctgc cacctgtgtg |
| 2581 | tctgaggggt g |

SEQ ID NO: 3 Human TP53 transcript variant 2 cDNA sequence
(NM_001126112.2; CDS: 200-1381)

| | |
|---|---|
| 1 | gatgggattg gggttttccc ctcccatgtg ctcaagactg gcgctaaaag ttttgagctt |
| 61 | ctcaaaagtc tagagccacc gtccagggag caggtagctg ctgggctccg gggacacttt |
| 121 | gcgttcgggc tgggagcgtg ctttccacga cggtgacacg cttccctgga ttggccagac |
| 181 | tgccttccgg gtcactgcca tggaggagcc gcagtcagat cctagcgtcg agccccctct |
| 241 | gagtcaggaa acattttcag acctatggaa actacttcct gaaaacaacg ttctgtcccc |
| 301 | cttgccgtcc caagcaatgg atgatttgat gctgtccccg gacgatattg aacaatggtt |
| 361 | cactgaagac ccaggtccag atgaagctcc cagaatgcca gaggctgctc cccccgtggc |
| 421 | ccctgcacca gcagctccta caccggcggc ccctgcacca gccccctcct ggcccctgtc |
| 481 | atcttctgtc ccttcccaga aaacctacca gggcagctac ggtttccgtc tgggcttctt |
| 541 | gcattctggg acagccaagt ctgtgacttg cacgtactcc cctgccctca acaagatgtt |
| 601 | ttgccaactg gccaagacct gccctgtgca gctgtgggtt gattccacac ccccgcccgg |
| 661 | cacccgcgtc cgcgccatgg ccatctacaa gcagtcacag cacatgacgg aggttgtgag |
| 721 | gcgctgcccc caccatgagc gctgctcaga tagcgatggt ctggcccctc ctcagcatct |
| 781 | tatccgagtg gaaggaaatt tgcgtgtgga gtatttggat gacagaaaca cttttcgaca |
| 841 | tagtgtggtg gtgccctatg agccgcctga ggttggctct gactgtacca ccatccacta |
| 901 | caactacatg tgtaacagtt cctgcatggg cggcatgaac cggaggccca tcctcaccat |
| 961 | catcacactg gaagactcca gtggtaatct actgggacgg aacagctttg aggtgcgtgt |
| 1021 | ttgtgcctgt cctgggagag accggcgcac agaggaagag aatctccgca agaaagggga |
| 1081 | gcctcaccac gagctgcccc cagggagcac taagcgagca ctgcccaaca acaccagctc |
| 1141 | ctctccccag ccaaagaaga aaccactgga tggagaatat ttcacccttc agatccgtgg |
| 1201 | gcgtgagcgc ttcgagatgt tccgagagct gaatgaggcc ttggaactca aggatgccca |
| 1261 | ggctgggaag gagccagggg ggagcagggc tcactccagc cacctgaagt ccaaaaaggg |
| 1321 | tcagtctacc tcccgccata aaaaactcat gttcaagaca gaagggcctg actcagactg |
| 1381 | acattctcca cttcttgttc cccactgaca gcctcccacc cccatctctc cctcccctgc |
| 1441 | cattttgggt tttgggtctt tgaacccttg cttgcaatag gtgtgcgtca gaagcaccca |
| 1501 | ggacttccat ttgctttgtc ccggggctcc actgaacaag ttggcctgca ctggtgtttt |
| 1561 | gttgtgggga ggaggatggg gagtaggaca taccagctta gattttaagg tttttactgt |
| 1621 | gagggatgtt tgggagatgt aagaaatgtt cttgcagtta agggttagtt tacaatcagc |
| 1681 | cacattctag gtaggggccc acttcaccgt actaaccagg gaagctgtcc ctcactgttg |
| 1741 | aattttctct aacttcaagg cccatatctg tgaaatgctg gcatttgcac ctacctcaca |
| 1801 | gagtgcattg tgagggttaa tgaaataatg tacatctggc cttgaaacca ccttttatta |
| 1861 | catggggtct agaacttgac ccccttgagg gtgcttgttc cctctccctg ttggtcggtg |
| 1921 | ggttggtagt ttctacagtt gggcagctgg ttaggtagag ggagttgtca agtctctgct |
| 1981 | ggcccagcca aaccctgtct gacaacctct tggtgaacct tagtacctaa aaggaaatct |
| 2041 | caccccatcc cacaccctgg aggatttcat ctcttgtata tgatgatctg gatccaccaa |
| 2101 | gacttgtttt atgctcaggg tcaatttctt ttttcttttt tttttttttt tttctttttc |
| 2161 | tttgagactg ggtctcgctt tgttgcccag gctggagtgg agtggcgtga tcttggctta |
| 2221 | ctgcagcctt tgcctccccg gctcgagcag tcctgcctca gcctccggag tagctgggac |
| 2281 | cacaggttca tgccaccatg gccagccaac ttttgcatgt tttgtagaga tggggtctca |
| 2341 | cagtgttgcc caggctggtc tcaaactcct gggctcaggc gatccacctg tctcagcctc |
| 2401 | ccagagtgct gggattacaa ttgtgagcca ccacgtccag ctggaagggt caacatcttt |
| 2461 | tacattctgc aagcacatct gcattttcac cccacccttc ccctccttct ccctttttat |
| 2521 | atcccatttt tatatcgatc tcttatttta caataaaact ttgctgccac ctgtgtgtct |
| 2581 | gaggggtg |

SEQ ID NO: 4 Human TP53 isoform b Amino Acid Sequence (NP_001119586.1)

| | |
|---|---|
| 1 | meepqsdpsv epplsqetfs dlwkllpenn vlsplpsqam ddlmlspddi eqwftedpgp |
| 61 | deaprmpeaa ppvapapaap tpaapapaps wplsssvpsq ktyqgsygfr lgflhsgtak |
| 121 | svtctyspal nkmfcqlakt cpvqlwvdst pppgtrvram aiykqsqhmt evvrrcphhe |
| 181 | rcsdsdglap pqhlirvegn lrveylddrn tfrhsvvvpy eppevgsdct tihynymcns |
| 241 | scmggmnrrp iltiitleds sgnllgrnsf evrvcacpgr drrteeenlr kkgephhelp |
| 301 | pgstkralpn ntssspqpkk kpldgeyftl qdqtsfqken c |

SEQ ID NO: 5 Human TP53 transcript variant 3 cDNA sequence
(NM_001126114.2; CDS: 203-1228)

| | |
|---|---|
| 1 | gatgggattg gggttttccc ctcccatgtg ctcaagactg gcgctaaaag ttttgagctt |
| 61 | ctcaaaagtc tagagccacc gtccagggag caggtagctg ctgggctccg gggacacttt |
| 121 | gcgttcgggc tgggagcgtg ctttccacga cggtgacacg cttccctgga ttggcagcca |
| 181 | gactgccttc cgggtcactg ccatggagga gccgcagtca gatcctagcg tcgagccccc |
| 241 | tctgagtcag gaaacatttt cagacctatg gaaactactt cctgaaaaca acgttctgtc |
| 301 | ccccttgccg tcccaagcaa tggatgattt gatgctgtcc ccggacgata ttgaacaatg |
| 361 | gttcactgaa gacccaggtc cagatgaagc tcccagaatg ccagaggctg ctccccccgt |
| 421 | ggcccctgca ccagcagctc ctacaccggc ggcccctgca ccagccccct cctggcccct |
| 481 | gtcatcttct gtcccttccc agaaaaccta ccagggcagc tacggtttcc gtctgggctt |
| 541 | cttgcattct gggacagcca agtctgtgac ttgcacgtac tcccctgccc tcaacaagat |
| 601 | gttttgccaa ctggccaaga cctgccctgt gcagctgtgg gttgattcca cacccccgcc |
| 661 | cggcacccgc gtccgcgcca tggccatcta caagcagtca cagcacatga cggaggttgt |
| 721 | gaggcgctgc ccccaccatg agcgctgctc agatagcgat ggtctggccc ctcctcagca |
| 781 | tcttatccga gtggaaggaa atttgcgtgt ggagtatttg gatgacagaa acacttttcg |
| 841 | acatagtgtg gtggtgccct atgagccgcc tgaggttggc tctgactgta ccaccatcca |
| 901 | ctacaactac atgtgtaaca gttcctgcat gggcggcatg aaccggaggc ccatcctcac |
| 961 | catcatcaca ctggaagact ccagtggtaa tctactggga cggaacagct ttgaggtgcg |
| 1021 | tgtttgtgcc tgtcctggga gagaccggcg cacagaggaa gagaatctcc gcaagaaagg |
| 1081 | ggagcctcac cacgagctgc ccccagggag cactaagcga gcactgccca acaacaccag |
| 1141 | ctcctctccc cagccaaaga agaaaccact ggatggagaa tatttcaccc ttcaggacca |
| 1201 | gaccagcttt caaaaagaaa attgttaaag agagcatgaa aatggttcta tgactttgcc |
| 1261 | tgatacagat gctacttgac ttacgatggt gttacttcct gataaactcg tcgtaagttg |
| 1321 | aaaatattat ccgtgggcgt gagcgcttcg agatgttccg agagctgaat gaggccttgg |
| 1381 | aactcaagga tgcccaggct gggaaggagc caggggggag cagggctcac tccagccacc |
| 1441 | tgaagtccaa aaagggtcag tctacctccc gccataaaaa actcatgttc aagacagaag |
| 1501 | ggcctgactc agactgacat tctccacttc ttgttcccca ctgacagcct cccaccccca |
| 1561 | tctctccctc ccctgccatt ttgggttttg ggtctttgaa cccttgcttg caataggtgt |
| 1621 | gcgtcagaag cacccaggac ttccatttgc tttgtcccgg ggctccactg aacaagttgg |
| 1681 | cctgcactgg tgttttgttg tggggaggag gatggggagt aggacatacc agcttagatt |
| 1741 | ttaaggtttt tactgtgagg gatgtttggg agatgtaaga aatgttcttg cagttaaggg |
| 1801 | ttagtttaca atcagccaca ttctaggtag gggcccactt caccgtacta accagggaag |
| 1861 | ctgtccctca ctgttgaatt ttctctaact tcaaggccca tatctgtgaa atgctggcat |
| 1921 | ttgcacctac ctcacagagt gcattgtgag ggttaatgaa ataatgtaca tctggccttg |
| 1981 | aaaccacctt ttattacatg gggtctagaa cttgaccccc ttgagggtgc ttgttccctc |
| 2041 | tccctgttgg tcggtgggtt ggtagtttct acagttgggc agctggttag gtagagggag |
| 2101 | ttgtcaagtc tctgctggcc cagccaaacc ctgtctgaca acctcttggt gaaccttagt |
| 2161 | acctaaaagg aaatctcacc ccatcccaca ccctggagga tttcatctct tgtatatgat |
| 2221 | gatctggatc caccaagact tgttttatgc tcagggtcaa tttctttttt cttttttttt |
| 2281 | tttttttttc tttttctttg agactgggtc tcgctttgtt gcccaggctg gagtggagtg |
| 2341 | gcgtgatctt ggcttactgc agcctttgcc tccccggctc gagcagtcct gcctcagcct |
| 2401 | ccggagtagc tgggaccaca ggttcatgcc accatggcca gccaactttt gcatgttttg |
| 2461 | tagagatggg gtctcacagt gttgcccagg ctggtctcaa actcctgggc tcaggcgatc |
| 2521 | cacctgtctc agcctcccag agtgctggga ttacaattgt gagccaccac gtccagctgg |
| 2581 | aagggtcaac atcttttaca ttctgcaagc acatctgcat tttcacccca cccttcccct |
| 2641 | ccttctccct ttttatatcc catttttata tcgatctctt attttacaat aaaactttgc |
| 2701 | tgccacctgt gtgtctgagg ggtg |

SEQ ID NO: 6 Human TP53 isoform c Amino Acid Sequence (NP_001119585.1)

| | |
|---|---|
| 1 | meepqsdpsv epplsqetfs dlwkllpenn vlsplpsqam ddlmlspddi eqwftedpgp |
| 61 | deaprmpeaa ppvapapaap tpaapapaps wplsssvpsq ktyqgsygfr lgflhsgtak |
| 121 | svtctyspal nkmfcqlakt cpvqlwvdst pppgtrvram aiykqsqhmt evvrrcphhe |
| 181 | rcsdsdglap pqhlirvegn lrveylddrn tfrhsvvvpy eppevgsdct tihynymcns |
| 241 | scmggmnrrp iltiitleds sgnllgrnsf evrvcacpgr drrteeenlr kkgephhelp |
| 301 | pgstkralpn ntssspqpkk kpldgeyftl qmlldlrwcy flinss |

SEQ ID NO: 7 Human TP53 transcript variant 4 cDNA sequence
(NM_001126113.2; CDS: 203-1243)

| | |
|---|---|
| 1 | gatgggattg gggttttccc ctcccatgtg ctcaagactg gcgctaaaag ttttgagctt |
| 61 | ctcaaaagtc tagagccacc gtccagggag caggtagctg ctgggctccg gggacacttt |
| 121 | gcgttcgggc tgggagcgtg ctttccacga cggtgacacg cttccctgga ttggcagcca |
| 181 | gactgccttc cgggtcactg ccatggagga gccgcagtca gatcctagcg tcgagccccc |
| 241 | tctgagtcag gaaacatttt cagacctatg gaaactactt cctgaaaaca acgttctgtc |
| 301 | ccccttgccg tcccaagcaa tggatgattt gatgctgtcc ccggacgata ttgaacaatg |
| 361 | gttcactgaa gacccaggtc cagatgaagc tcccagaatg ccagaggctg ctccccccgt |
| 421 | ggcccctgca ccagcagctc ctacaccggc ggcccctgca ccagccccct cctggcccct |
| 481 | gtcatcttct gtcccttccc agaaaaccta ccagggcagc tacggtttcc gtctgggctt |
| 541 | cttgcattct gggacagcca agtctgtgac ttgcacgtac tcccctgccc tcaacaagat |
| 601 | gttttgccaa ctggccaaga cctgccctgt gcagctgtgg gttgattcca cacccccgcc |
| 661 | cggcacccgc gtccgcgcca tggccatcta caagcagtca cagcacatga cggaggttgt |
| 721 | gaggcgctgc ccccaccatg agcgctgctc agatagcgat ggtctggccc ctcctcagca |
| 781 | tcttatccga gtggaaggaa atttgcgtgt ggagtatttg gatgacagaa acacttttcg |
| 841 | acatagtgtg gtggtgccct atgagccgcc tgaggttggc tctgactgta ccaccatcca |
| 901 | ctacaactac atgtgtaaca gttcctgcat gggcggcatg aaccggaggc ccatcctcac |
| 961 | catcatcaca ctggaagact ccagtggtaa tctactggga cggaacagct ttgaggtgcg |
| 1021 | tgtttgtgcc tgtcctggga gagaccggcg cacagaggaa gagaatctcc gcaagaaagg |
| 1081 | ggagcctcac cacgagctgc ccccagggag cactaagcga gcactgccca acaacaccag |
| 1141 | ctcctctccc cagccaaaga agaaaccact ggatggagaa tatttcaccc ttcagatgct |
| 1201 | acttgactta cgatggtgtt acttcctgat aaactcgtcg taagttgaaa atattatccg |
| 1261 | tgggcgtgag cgcttcgaga tgttccgaga gctgaatgag gccttggaac tcaaggatgc |
| 1321 | ccaggctggg aaggagccag gggggagcag ggctcactcc agccacctga agtccaaaaa |
| 1381 | gggtcagtct acctcccgcc ataaaaaact catgttcaag acagaagggc ctgactcaga |
| 1441 | ctgacattct ccacttcttg ttccccactg acagcctccc acccccatct ctccctcccc |
| 1501 | tgccattttg ggttttgggt ctttgaaccc ttgcttgcaa taggtgtgcg tcagaagcac |
| 1561 | ccaggacttc catttgcttt gtcccggggc tccactgaac aagttggcct gcactggtgt |
| 1621 | tttgttgtgg ggaggaggat ggggagtagg acataccagc ttagatttta aggtttttac |
| 1681 | tgtgagggat gtttgggaga tgtaagaaat gttcttgcag ttaagggtta gtttacaatc |
| 1741 | agccacattc taggtagggg cccacttcac cgtactaacc agggaagctg tccctcactg |
| 1801 | ttgaattttc tctaacttca aggcccatat ctgtgaaatg ctggcatttg cacctacctc |
| 1861 | acagagtgca ttgtgagggt taatgaaata atgtacatct ggccttgaaa ccacctttta |
| 1921 | ttacatgggg tctagaactt gacccccttg agggtgcttg ttccctctcc ctgttggtcg |
| 1981 | gtgggttggt agtttctaca gttgggcagc tggttaggta gagggagttg tcaagtctct |
| 2041 | gctggcccag ccaaaccctg tctgacaacc tcttggtgaa ccttagtacc taaaaggaaa |
| 2101 | tctcacccca tcccacaccc tggaggattt catctcttgt atatgatgat ctggatccac |
| 2161 | caagacttgt tttatgctca gggtcaattt cttttttctt tttttttttt ttttttcttt |
| 2221 | ttctttgaga ctgggtctcg ctttgttgcc caggctggag tggagtggcg tgatcttggc |
| 2281 | ttactgcagc ctttgcctcc ccggctcgag cagtcctgcc tcagcctccg gagtagctgg |
| 2341 | gaccacaggt tcatgccacc atggccagcc aacttttgca tgttttgtag agatggggtc |
| 2401 | tcacagtgtt gcccaggctg gtctcaaact cctgggctca ggcgatccac ctgtctcagc |
| 2461 | ctcccagagt gctgggatta caattgtgag ccaccacgtc cagctggaag ggtcaacatc |
| 2521 | ttttacattc tgcaagcaca tctgcatttt caccccaccc ttcccctcct tctccctttt |
| 2581 | tatatcccat ttttatatcg atctcttatt ttacaataaa actttgctgc cacctgtgtg |
| 2641 | tctgaggggt g |

SEQ ID NO: 8 Human TP53 isoform d Amino Acid Sequence (NP_001119587.1)

| | |
|---|---|
| 1 | mfcqlaktcp vqlwvdstpp pgtrvramai ykqsqhmtev vrrcphherc sdsdglappq |
| 61 | hlirvegnlr veylddrntf rhsvvvpyep pevgsdctti hynymcnssc mggmnrrpil |
| 121 | tiitledssg nllgrnsfev rvcacpgrdr rteeenlrkk gephhelppg stkralpnnt |
| 181 | ssspqpkkkp ldgeyftlqi rgrerfemfr elnealelkd aqagkepggs rahsshlksk |
| 241 | kgqstsrhkk lmfktegpds d |

SEQ ID NO: 9 Human TP53 transcript variant 5 cDNA sequence
(NM_001126115.1; CDS: 279-1064)

| | |
|---|---|
| 1 | tgaggccagg agatggaggc tgcagtgagc tgtgatcaca ccactgtgct ccagcctgag |
| 61 | tgacagagca agaccctatc tcaaaaaaaa aaaaaaaaaa gaaaagctcc tgaggtgtag |
| 121 | acgccaactc tctctagctc gctagtgggt tgcaggaggt gcttacgcat gtttgtttct |
| 181 | ttgctgccgt cttccagttg ctttatctgt tcacttgtgc cctgactttc aactctgtct |
| 241 | ccttcctctt cctacagtac tcccctgccc tcaacaagat gttttgccaa ctggccaaga |
| 301 | cctgccctgt gcagctgtgg gttgattcca cacccccgcc cggcacccgc gtccgcgcca |
| 361 | tggccatcta caagcagtca cagcacatga cggaggttgt gaggcgctgc ccccaccatg |
| 421 | agcgctgctc agatagcgat ggtctggccc ctcctcagca tcttatccga gtggaaggaa |
| 481 | atttgcgtgt ggagtatttg gatgacagaa acacttttcg acatagtgtg gtggtgccct |
| 541 | atgagccgcc tgaggttggc tctgactgta ccaccatcca ctacaactac atgtgtaaca |
| 601 | gttcctgcat gggcggcatg aaccggaggc ccatcctcac catcatcaca ctggaagact |
| 661 | ccagtggtaa tctactggga cggaacagct ttgaggtgcg tgtttgtgcc tgtcctggga |
| 721 | gagaccggcg cacagaggaa gagaatctcc gcaagaaagg ggagcctcac cacgagctgc |
| 781 | ccccagggag cactaagcga gcactgccca acaacaccag ctcctctccc cagccaaaga |
| 841 | agaaaccact ggatggagaa tatttcaccc ttcagatccg tgggcgtgag cgcttcgaga |
| 901 | tgttccgaga gctgaatgag gccttggaac tcaaggatgc ccaggctggg aaggagccag |
| 961 | gggggagcag ggctcactcc agccacctga agtccaaaaa gggtcagtct acctcccgcc |
| 1021 | ataaaaaact catgttcaag acagaagggc ctgactcaga ctgacattct ccacttcttg |
| 1081 | ttccccactg acagcctccc acccccatct ctccctcccc tgccattttg ggttttgggt |
| 1141 | ctttgaaccc ttgcttgcaa taggtgtgcg tcagaagcac ccaggacttc catttgcttt |
| 1201 | gtcccggggc tccactgaac aagttggcct gcactggtgt tttgttgtgg ggaggaggat |
| 1261 | ggggagtagg acataccagc ttagatttta aggtttttac tgtgagggat gtttgggaga |
| 1321 | tgtaagaaat gttcttgcag ttaagggtta gtttacaatc agccacattc taggtagggg |
| 1381 | cccacttcac cgtactaacc agggaagctg tccctcactg ttgaattttc tctaacttca |
| 1441 | aggcccatat ctgtgaaatg ctggcatttg cacctacctc acagagtgca ttgtgagggt |
| 1501 | taatgaaata atgtacatct ggccttgaaa ccacctttta ttacatgggg tctagaactt |
| 1561 | gacccccttg agggtgcttg ttccctctcc ctgttggtcg gtgggttggt agtttctaca |
| 1621 | gttgggcagc tggttaggta gagggagttg tcaagtctct gctggcccag ccaaaccctg |
| 1681 | tctgacaacc tcttggtgaa ccttagtacc taaaaggaaa tctcacccca tcccacaccc |
| 1741 | tggaggattt catctcttgt atatgatgat ctggatccac caagacttgt tttatgctca |
| 1801 | gggtcaattt cttttttctt tttttttttt ttttttcttt ttctttgaga ctgggtctcg |
| 1861 | ctttgttgcc caggctggag tggagtggcg tgatcttggc ttactgcagc ctttgcctcc |
| 1921 | ccggctcgag cagtcctgcc tcagcctccg gagtagctgg gaccacaggt tcatgccacc |
| 1981 | atggccagcc aacttttgca tgttttgtag agatggggtc tcacagtgtt gcccaggctg |
| 2041 | gtctcaaact cctgggctca ggcgatccac ctgtctcagc ctcccagagt gctgggatta |
| 2101 | caattgtgag ccaccacgtc cagctggaag ggtcaacatc ttttacattc tgcaagcaca |
| 2161 | tctgcatttt caccccaccc ttcccctcct tctccctttt tatatcccat ttttatatcg |
| 2221 | atctcttatt ttacaataaa actttgctgc cacctgtgtg tctgaggggt g |
| 1981 | atggccagcc aacttttgca tgttttgtag agatggggtc tcacagtgtt gcccaggctg |

SEQ ID NO: 10 Human TP53 isoform e Amino Acid Sequence (NP_001119588.1)

| | |
|---|---|
| 1 | mfcqlaktcp vqlwvdstpp pgtrvramai ykqsqhmtev vrrcphherc sdsdglappq |
| 61 | hlirvegnlr veylddrntf rhsvvvpyep pevgsdctti hynymcnssc mggmnrrpil |
| 121 | tiitledssg nllgrnsfev rvcacpgrdr rteeenlrkk gephhelppg stkralpnnt |
| 181 | ssspqpkkkp ldgeyftlqd qtsfqkenc |

SEQ ID NO: 11 Human TP53 transcript variant 6 cDNA sequence
(NM_001126116.1; CDS: 279-908)

| | |
|---|---|
| 1 | tgaggccagg agatggaggc tgcagtgagc tgtgatcaca ccactgtgct ccagcctgag |
| 61 | tgacagagca agaccctatc tcaaaaaaaa aaaaaaaaaa gaaaagctcc tgaggtgtag |
| 121 | acgccaactc tctctagctc gctagtgggt tgcaggaggt gcttacgcat gtttgtttct |
| 181 | ttgctgccgt cttccagttg ctttatctgt tcacttgtgc cctgactttc aactctgtct |
| 241 | ccttcctctt cctacagtac tcccctgccc tcaacaagat gttttgccaa ctggccaaga |
| 301 | cctgccctgt gcagctgtgg gttgattcca cacccccgcc cggcacccgc gtccgcgcca |
| 361 | tggccatcta caagcagtca cagcacatga cggaggttgt gaggcgctgc ccccaccatg |
| 421 | agcgctgctc agatagcgat ggtctggccc ctcctcagca tcttatccga gtggaaggaa |
| 481 | atttgcgtgt ggagtatttg gatgacagaa acacttttcg acatagtgtg gtggtgccct |
| 541 | atgagccgcc tgaggttggc tctgactgta ccaccatcca ctacaactac atgtgtaaca |
| 601 | gttcctgcat gggcggcatg aaccggaggc ccatcctcac catcatcaca ctggaagact |
| 661 | ccagtggtaa tctactggga cggaacagct ttgaggtgcg tgtttgtgcc tgtcctggga |
| 721 | gagaccggcg cacagaggaa gagaatctcc gcaagaaagg ggagcctcac cacgagctgc |
| 781 | ccccagggag cactaagcga gcactgccca acaacaccag ctcctctccc cagccaaaga |
| 841 | agaaaccact ggatggagaa tatttcaccc ttcaggacca gaccagcttt caaaaagaaa |
| 901 | attgttaaag agagcatgaa aatggttcta tgactttgcc tgatacagat gctacttgac |
| 961 | ttacgatggt gttacttcct gataaactcg tcgtaagttg aaaatattat ccgtgggcgt |
| 1021 | gagcgcttcg agatgttccg agagctgaat gaggccttgg aactcaagga tgcccaggct |
| 1081 | gggaaggagc caggggggag cagggctcac tccagccacc tgaagtccaa aaagggtcag |
| 1141 | tctacctccc gccataaaaa actcatgttc aagacagaag ggcctgactc agactgacat |
| 1201 | tctccacttc ttgttcccca ctgacagcct cccaccccca tctctccctc ccctgccatt |
| 1261 | ttgggttttg ggtctttgaa cccttgcttg caataggtgt gcgtcagaag cacccaggac |
| 1321 | ttccatttgc tttgtcccgg ggctccactg aacaagttgg cctgcactgg tgttttgttg |
| 1381 | tggggaggag gatggggagt aggacatacc agcttagatt ttaaggtttt tactgtgagg |
| 1441 | gatgtttggg agatgtaaga aatgttcttg cagttaaggg ttagtttaca atcagccaca |
| 1501 | ttctaggtag gggcccactt caccgtacta accagggaag ctgtccctca ctgttgaatt |
| 1561 | ttctctaact tcaaggccca tatctgtgaa atgctggcat ttgcacctac ctcacagagt |
| 1621 | gcattgtgag ggttaatgaa ataatgtaca tctggccttg aaaccacctt ttattacatg |
| 1681 | gggtctagaa cttgaccccc ttgagggtgc ttgttccctc tccctgttgg tcggtgggtt |
| 1741 | ggtagtttct acagttgggc agctggttag gtagagggag ttgtcaagtc tctgctggcc |
| 1801 | cagccaaacc ctgtctgaca acctcttggt gaaccttagt acctaaaagg aaatctcacc |
| 1861 | ccatcccaca ccctggagga tttcatctct tgtatatgat gatctggatc caccaagact |
| 1921 | tgttttatgc tcagggtcaa tttctttttt cttttttttt tttttttttc tttttctttg |
| 1981 | agactgggtc tcgctttgtt gcccaggctg gagtggagtg gcgtgatctt ggcttactgc |
| 2041 | agcctttgcc tccccggctc gagcagtcct gcctcagcct ccggagtagc tgggaccaca |
| 2101 | ggttcatgcc accatggcca gccaactttt gcatgttttg tagagatggg gtctcacagt |
| 2161 | gttgcccagg ctggtctcaa actcctgggc tcaggcgatc cacctgtctc agcctcccag |
| 2221 | agtgctggga ttacaattgt gagccaccac gtccagctgg aagggtcaac atcttttaca |
| 2281 | ttctgcaagc acatctgcat tttcacccca cccttcccct ccttctccct ttttatatcc |
| 2341 | catttttata tcgatctctt attttacaat aaaactttgc tgccacctgt gtgtctgagg |
| 2401 | ggtg |

SEQ ID NO: 12 Human TP53 isoform f Amino Acid Sequence (NP_001119589.1)

| | |
|---|---|
| 1 | mfcqlaktcp vqlwvdstpp pgtrvramai ykqsqhmtev vrrcphherc sdsdglappq |
| 61 | hlirvegnlr veylddrntf rhsvvvpyep pevgsdctti hynymcnssc mggmnrrpil |
| 121 | tiitledssg nllgrnsfev rvcacpgrdr rteeenlrkk gephhelppg stkralpnnt |
| 181 | ssspqpkkkp ldgeyftlqm lldlrwcyfl inss |

SEQ ID NO: 13 Human TP53 transcript variant 7 cDNA sequence
(NM_001126117.1; CDS: 279-923)

| | |
|---|---|
| 1 | tgaggccagg agatggaggc tgcagtgagc tgtgatcaca ccactgtgct ccagcctgag |
| 61 | tgacagagca agaccctatc tcaaaaaaaa aaaaaaaaaa gaaaagctcc tgaggtgtag |
| 121 | acgccaactc tctctagctc gctagtgggt tgcaggaggt gcttacgcat gtttgtttct |
| 181 | ttgctgccgt cttccagttg ctttatctgt tcacttgtgc cctgactttc aactctgtct |
| 241 | ccttcctctt cctacagtac tcccctgccc tcaacaagat gttttgccaa ctggccaaga |
| 301 | cctgccctgt gcagctgtgg gttgattcca cacccccgcc cggcacccgc gtccgcgcca |
| 361 | tggccatcta caagcagtca cagcacatga cggaggttgt gaggcgctgc ccccaccatg |
| 421 | agcgctgctc agatagcgat ggtctggccc ctcctcagca tcttatccga gtggaaggaa |
| 481 | atttgcgtgt ggagtatttg gatgacagaa acacttttcg acatagtgtg gtggtgccct |
| 541 | atgagccgcc tgaggttggc tctgactgta ccaccatcca ctacaactac atgtgtaaca |
| 601 | gttcctgcat gggcggcatg aaccggaggc ccatcctcac catcatcaca ctggaagact |
| 661 | ccagtggtaa tctactggga cggaacagct ttgaggtgcg tgtttgtgcc tgtcctggga |
| 721 | gagaccggcg cacagaggaa gagaatctcc gcaagaaagg ggagcctcac cacgagctgc |
| 781 | ccccagggag cactaagcga gcactgccca acaacaccag ctcctctccc cagccaaaga |
| 841 | agaaaccact ggatggagaa tatttcaccc ttcagatgct acttgactta cgatggtgtt |
| 901 | acttcctgat aaactcgtcg taagttgaaa atattatccg tgggcgtgag cgcttcgaga |
| 961 | tgttccgaga gctgaatgag gccttggaac tcaaggatgc ccaggctggg aaggagccag |
| 1021 | gggggagcag ggctcactcc agccacctga agtccaaaaa gggtcagtct acctcccgcc |
| 1081 | ataaaaaact catgttcaag acagaagggc ctgactcaga ctgacattct ccacttcttg |
| 1141 | ttccccactg acagcctccc acccccatct ctccctcccc tgccattttg ggttttgggt |
| 1201 | ctttgaaccc ttgcttgcaa taggtgtgcg tcagaagcac ccaggacttc catttgcttt |
| 1261 | gtcccggggc tccactgaac aagttggcct gcactggtgt tttgttgtgg ggaggaggat |
| 1321 | ggggagtagg acataccagc ttagatttta aggtttttac tgtgagggat gtttgggaga |
| 1381 | tgtaagaaat gttcttgcag ttaagggtta gtttacaatc agccacattc taggtagggg |
| 1441 | cccacttcac cgtactaacc agggaagctg tccctcactg ttgaattttc tctaacttca |
| 1501 | aggcccatat ctgtgaaatg ctggcatttg cacctacctc acagagtgca ttgtgagggt |
| 1561 | taatgaaata atgtacatct ggccttgaaa ccacctttta ttacatgggg tctagaactt |
| 1621 | gacccccttg agggtgcttg ttccctctcc ctgttggtcg gtgggttggt agtttctaca |
| 1681 | gttgggcagc tggttaggta gagggagttg tcaagtctct gctggcccag ccaaaccctg |
| 1741 | tctgacaacc tcttggtgaa ccttagtacc taaaaggaaa tctcacccca tcccacaccc |
| 1801 | tggaggattt catctcttgt atatgatgat ctggatccac caagacttgt tttatgctca |
| 1861 | gggtcaattt cttttttctt tttttttttt ttttttcttt ttctttgaga ctgggtctcg |
| 1921 | ctttgttgcc caggctggag tggagtggcg tgatcttggc ttactgcagc ctttgcctcc |
| 1981 | ccggctcgag cagtcctgcc tcagcctccg gagtagctgg gaccacaggt tcatgccacc |
| 2041 | atggccagcc aacttttgca tgttttgtag agatggggtc tcacagtgtt gcccaggctg |
| 2101 | gtctcaaact cctgggctca ggcgatccac ctgtctcagc ctcccagagt gctgggatta |
| 2161 | caattgtgag ccaccacgtc cagctggaag ggtcaacatc ttttacattc tgcaagcaca |
| 2221 | tctgcatttt caccccaccc ttcccctcct tctccctttt tatatcccat ttttatatcg |
| 2281 | atctcttatt ttacaataaa actttgctgc cacctgtgtg tctgaggggt g |

SEQ ID NO: 14 Human TP53 isoform g Amino Acid Sequence (NP_001119590.1,
NP_001263689,1, and NP_001263690.1)

| | |
|---|---|
| 1 | mddlmlspdd ieqwftedpg pdeaprmpea appvapapaa ptpaapapap swplsssvps |
| 61 | qktyqgsygf rlgflhsgta ksvtctyspa lnkmfcqlak tcpvqlwvds tpppgtrvra |
| 121 | maiykqsqhm tevvrrcphh ercsdsdgla ppqhlirveg nlrveylddr ntfrhsvvvp |
| 181 | yeppevgsdc ttihynymcn sscmggmnrr piltiitled ssgnllgrns fevrvcacpg |
| 241 | rdrrteeenl rkkgephhel ppgstkralp nntssspqpk kkpldgeyft lqirgrerfe |
| 301 | mfrelneale lkdaqagkep ggsrahsshl kskkgqstsr hkklmfkteg pdsd |

SEQ ID NO: 15 Human TP53 transcript variant 8 cDNA sequence
(NM_001126118.1; CDS: 437-1501)

| | |
|---|---|
| 1 | gatgggattg gggttttccc ctcccatgtg ctcaagactg gcgctaaaag ttttgagctt |
| 61 | ctcaaaagtc tagagccacc gtccagggag caggtagctg ctgggctccg gggacacttt |
| 121 | gcgttcgggc tgggagcgtg ctttccacga cggtgacacg cttccctgga ttggcagcca |
| 181 | gactgccttc cgggtcactg ccatggagga gccgcagtca gatcctagcg tcgagccccc |
| 241 | tctgagtcag gaaacatttt cagacctatg gaaactgtga gtggatccat tggaagggca |
| 301 | ggcccaccac ccccacccca accccagccc cctagcagag acctgtggga agcgaaaatt |
| 361 | ccatgggact gactttctgc tcttgtcttt cagacttcct gaaaacaacg ttctgtcccc |
| 421 | cttgccgtcc caagcaatgg atgatttgat gctgtccccg gacgatattg aacaatggtt |
| 481 | cactgaagac ccaggtccag atgaagctcc cagaatgcca gaggctgctc cccccgtggc |
| 541 | ccctgcacca gcagctccta caccggcggc ccctgcacca gccccctcct ggcccctgtc |
| 601 | atcttctgtc ccttcccaga aaacctacca gggcagctac ggtttccgtc tgggcttctt |
| 661 | gcattctggg acagccaagt ctgtgacttg cacgtactcc cctgccctca acaagatgtt |
| 721 | ttgccaactg gccaagacct gccctgtgca gctgtgggtt gattccacac ccccgcccgg |
| 781 | cacccgcgtc cgcgccatgg ccatctacaa gcagtcacag cacatgacgg aggttgtgag |
| 841 | gcgctgcccc caccatgagc gctgctcaga tagcgatggt ctggcccctc ctcagcatct |
| 901 | tatccgagtg gaaggaaatt tgcgtgtgga gtatttggat gacagaaaca cttttcgaca |
| 961 | tagtgtggtg gtgccctatg agccgcctga ggttggctct gactgtacca ccatccacta |
| 1021 | caactacatg tgtaacagtt cctgcatggg cggcatgaac cggaggccca tcctcaccat |
| 1081 | catcacactg gaagactcca gtggtaatct actgggacgg aacagctttg aggtgcgtgt |
| 1141 | ttgtgcctgt cctgggagag accggcgcac agaggaagag aatctccgca agaaagggga |
| 1201 | gcctcaccac gagctgcccc cagggagcac taagcgagca ctgcccaaca acaccagctc |
| 1261 | ctctccccag ccaaagaaga aaccactgga tggagaatat ttcacccttc agatccgtgg |
| 1321 | gcgtgagcgc ttcgagatgt tccgagagct gaatgaggcc ttggaactca aggatgccca |
| 1381 | ggctgggaag gagccagggg ggagcagggc tcactccagc cacctgaagt ccaaaaaggg |
| 1441 | tcagtctacc tcccgccata aaaaactcat gttcaagaca gaagggcctg actcagactg |
| 1501 | acattctcca cttcttgttc cccactgaca gcctcccacc cccatctctc cctcccctgc |
| 1561 | cattttgggt tttgggtctt tgaacccttg cttgcaatag gtgtgcgtca gaagcaccca |
| 1621 | ggacttccat ttgctttgtc ccggggctcc actgaacaag ttggcctgca ctggtgtttt |
| 1681 | gttgtgggga ggaggatggg gagtaggaca taccagctta gattttaagg tttttactgt |
| 1741 | gagggatgtt tgggagatgt aagaaatgtt cttgcagtta agggttagtt tacaatcagc |
| 1801 | cacattctag gtaggggccc acttcaccgt actaaccagg gaagctgtcc ctcactgttg |
| 1861 | aattttctct aacttcaagg cccatatctg tgaaatgctg gcatttgcac ctacctcaca |
| 1921 | gagtgcattg tgagggttaa tgaaataatg tacatctggc cttgaaacca ccttttatta |
| 1981 | catggggtct agaacttgac ccccttgagg gtgcttgttc cctctccctg ttggtcggtg |
| 2041 | ggttggtagt ttctacagtt gggcagctgg ttaggtagag ggagttgtca agtctctgct |
| 2101 | ggcccagcca aaccctgtct gacaacctct tggtgaacct tagtacctaa aaggaaatct |
| 2161 | caccccatcc cacaccctgg aggatttcat ctcttgtata tgatgatctg gatccaccaa |
| 2221 | gacttgtttt atgctcaggg tcaatttctt ttttcttttt tttttttttt tttctttttc |
| 2281 | tttgagactg ggtctcgctt tgttgcccag gctggagtgg agtggcgtga tcttggctta |
| 2341 | ctgcagcctt tgcctccccg gctcgagcag tcctgcctca gcctccggag tagctgggac |
| 2401 | cacaggttca tgccaccatg gccagccaac ttttgcatgt tttgtagaga tggggtctca |
| 2461 | cagtgttgcc caggctggtc tcaaactcct gggctcaggc gatccacctg tctcagcctc |
| 2521 | ccagagtgct gggattacaa ttgtgagcca ccacgtccag ctggaagggt caacatcttt |
| 2581 | tacattctgc aagcacatct gcattttcac cccacccttc ccctccttct ccctttttat |
| 2641 | atcccatttt tatatcgatc tcttatttta caataaaact ttgctgccac ctgtgtgtct |
| 2701 | gaggggtg |

SEQ ID NO: 16 Human TP53 transcript variant 1 cDNA Sequence
(NM_001276760.1; CDS: 320-1384)

| | |
|---|---|
| 1 | gatgggattg gggttttccc ctcccatgtg ctcaagactg gcgctaaaag ttttgagctt |
| 61 | ctcaaaagtc tagagccacc gtccagggag caggtagctg ctgggctccg gggacacttt |
| 121 | gcgttcgggc tgggagcgtg ctttccacga cggtgacacg cttccctgga ttggcagcca |
| 181 | gactgccttc cgggtcactg ccatggagga gccgcagtca gatcctagcg tcgagccccc |
| 241 | tctgagtcag gaaacatttt cagacctatg gaaactactt cctgaaaaca acgttctgtc |
| 301 | ccccttgccg tcccaagcaa tggatgattt gatgctgtcc ccggacgata ttgaacaatg |
| 361 | gttcactgaa gacccaggtc cagatgaagc tcccagaatg ccagaggctg ctccccccgt |
| 421 | ggcccctgca ccagcagctc ctacaccggc ggcccctgca ccagccccct cctggcccct |
| 481 | gtcatcttct gtcccttccc agaaaaccta ccagggcagc tacggtttcc gtctgggctt |
| 541 | cttgcattct gggacagcca agtctgtgac ttgcacgtac tcccctgccc tcaacaagat |
| 601 | gttttgccaa ctggccaaga cctgccctgt gcagctgtgg gttgattcca cacccccgcc |
| 661 | cggcacccgc gtccgcgcca tggccatcta caagcagtca cagcacatga cggaggttgt |
| 721 | gaggcgctgc ccccaccatg agcgctgctc agatagcgat ggtctggccc ctcctcagca |
| 781 | tcttatccga gtggaaggaa atttgcgtgt ggagtatttg gatgacagaa acacttttcg |
| 841 | acatagtgtg gtggtgccct atgagccgcc tgaggttggc tctgactgta ccaccatcca |
| 901 | ctacaactac atgtgtaaca gttcctgcat gggcggcatg aaccggaggc ccatcctcac |
| 961 | catcatcaca ctggaagact ccagtggtaa tctactggga cggaacagct ttgaggtgcg |
| 1021 | tgtttgtgcc tgtcctggga gagaccggcg cacagaggaa gagaatctcc gcaagaaagg |
| 1081 | ggagcctcac cacgagctgc ccccagggag cactaagcga gcactgccca acaacaccag |
| 1141 | ctcctctccc cagccaaaga agaaaccact ggatggagaa tatttcaccc ttcagatccg |
| 1201 | tgggcgtgag cgcttcgaga tgttccgaga gctgaatgag gccttggaac tcaaggatgc |
| 1261 | ccaggctggg aaggagccag gggggagcag ggctcactcc agccacctga agtccaaaaa |
| 1321 | gggtcagtct acctcccgcc ataaaaaact catgttcaag acagaagggc ctgactcaga |
| 1381 | ctgacattct ccacttcttg ttccccactg acagcctccc acccccatct ctccctcccc |
| 1441 | tgccattttg ggttttgggt ctttgaaccc ttgcttgcaa taggtgtgcg tcagaagcac |
| 1501 | ccaggacttc catttgcttt gtcccggggc tccactgaac aagttggcct gcactggtgt |
| 1561 | tttgttgtgg ggaggaggat ggggagtagg acataccagc ttagatttta aggtttttac |
| 1621 | tgtgagggat gtttgggaga tgtaagaaat gttcttgcag ttaagggtta gtttacaatc |
| 1681 | agccacattc taggtagggg cccacttcac cgtactaacc agggaagctg tccctcactg |
| 1741 | ttgaattttc tctaacttca aggcccatat ctgtgaaatg ctggcatttg cacctacctc |
| 1801 | acagagtgca ttgtgagggt taatgaaata atgtacatct ggccttgaaa ccacctttta |
| 1861 | ttacatgggg tctagaactt gacccccttg agggtgcttg ttccctctcc ctgttggtcg |
| 1921 | gtgggttggt agtttctaca gttgggcagc tggttaggta gagggagttg tcaagtctct |
| 1981 | gctggcccag ccaaaccctg tctgacaacc tcttggtgaa ccttagtacc taaaaggaaa |
| 2041 | tctcacccca tcccacaccc tggaggattt catctcttgt atatgatgat ctggatccac |
| 2101 | caagacttgt tttatgctca gggtcaattt cttttttctt tttttttttt ttttttcttt |
| 2161 | ttctttgaga ctgggtctcg ctttgttgcc caggctggag tggagtggcg tgatcttggc |
| 2221 | ttactgcagc ctttgcctcc ccggctcgag cagtcctgcc tcagcctccg gagtagctgg |
| 2281 | gaccacaggt tcatgccacc atggccagcc aacttttgca tgttttgtag agatggggtc |
| 2341 | tcacagtgtt gcccaggctg gtctcaaact cctgggctca ggcgatccac ctgtctcagc |
| 2401 | ctcccagagt gctgggatta caattgtgag ccaccacgtc cagctggaag ggtcaacatc |
| 2461 | ttttacattc tgcaagcaca tctgcatttt caccccaccc ttcccctcct tctccctttt |
| 2521 | tatatcccat ttttatatcg atctcttatt ttacaataaa actttgctgc cacctgtgtg |
| 2581 | tctgaggggt g |

SEQ ID NO: 17 Human TP53 transcript variant 2 cDNA Sequence
(NM_001276761.1; CDS:317-1381)

| | |
|---|---|
| 1 | gatgggattg gggttttccc ctcccatgtg ctcaagactg gcgctaaaag ttttgagctt |
| 61 | ctcaaaagtc tagagccacc gtccagggag caggtagctg ctgggctccg gggacacttt |
| 121 | gcgttcgggc tgggagcgtg ctttccacga cggtgacacg cttccctgga ttggccagac |
| 181 | tgccttccgg gtcactgcca tggaggagcc gcagtcagat cctagcgtcg agccccctct |
| 241 | gagtcaggaa acattttcag acctatggaa actacttcct gaaaacaacg ttctgtcccc |
| 301 | cttgccgtcc caagcaatgg atgatttgat gctgtccccg gacgatattg aacaatggtt |
| 361 | cactgaagac ccaggtccag atgaagctcc cagaatgcca gaggctgctc cccccgtggc |
| 421 | ccctgcacca gcagctccta caccggcggc ccctgcacca gccccctcct ggcccctgtc |
| 481 | atcttctgtc ccttcccaga aaacctacca gggcagctac ggtttccgtc tgggcttctt |
| 541 | gcattctggg acagccaagt ctgtgacttg cacgtactcc cctgccctca acaagatgtt |
| 601 | ttgccaactg gccaagacct gccctgtgca gctgtgggtt gattccacac ccccgcccgg |
| 661 | cacccgcgtc cgcgccatgg ccatctacaa gcagtcacag cacatgacgg aggttgtgag |
| 721 | gcgctgcccc caccatgagc gctgctcaga tagcgatggt ctggcccctc ctcagcatct |
| 781 | tatccgagtg gaaggaaatt tgcgtgtgga gtatttggat gacagaaaca cttttcgaca |
| 841 | tagtgtggtg gtgccctatg agccgcctga ggttggctct gactgtacca ccatccacta |
| 901 | caactacatg tgtaacagtt cctgcatggg cggcatgaac cggaggccca tcctcaccat |
| 961 | catcacactg gaagactcca gtggtaatct actgggacgg aacagctttg aggtgcgtgt |
| 1021 | ttgtgcctgt cctgggagag accggcgcac agaggaagag aatctccgca agaaagggga |
| 1081 | gcctcaccac gagctgcccc cagggagcac taagcgagca ctgcccaaca acaccagctc |
| 1141 | ctctccccag ccaaagaaga aaccactgga tggagaatat ttcacccttc agatccgtgg |
| 1201 | gcgtgagcgc ttcgagatgt tccgagagct gaatgaggcc ttggaactca aggatgccca |
| 1261 | ggctgggaag gagccagggg ggagcagggc tcactccagc cacctgaagt ccaaaaaggg |
| 1321 | tcagtctacc tcccgccata aaaaactcat gttcaagaca gaagggcctg actcagactg |
| 1381 | acattctcca cttcttgttc cccactgaca gcctcccacc cccatctctc cctcccctgc |
| 1441 | cattttgggt tttgggtctt tgaacccttg cttgcaatag gtgtgcgtca gaagcaccca |
| 1501 | ggacttccat ttgctttgtc ccggggctcc actgaacaag ttggcctgca ctggtgtttt |
| 1561 | gttgtgggga ggaggatggg gagtaggaca taccagctta gattttaagg tttttactgt |
| 1621 | gagggatgtt tgggagatgt aagaaatgtt cttgcagtta agggttagtt tacaatcagc |
| 1681 | cacattctag gtaggggccc acttcaccgt actaaccagg gaagctgtcc ctcactgttg |
| 1741 | aattttctct aacttcaagg cccatatctg tgaaatgctg gcatttgcac ctacctcaca |
| 1801 | gagtgcattg tgagggttaa tgaaataatg tacatctggc cttgaaacca ccttttatta |
| 1861 | catggggtct agaacttgac ccccttgagg gtgcttgttc cctctccctg ttggtcggtg |
| 1921 | ggttggtagt ttctacagtt gggcagctgg ttaggtagag ggagttgtca agtctctgct |
| 1981 | ggcccagcca aaccctgtct gacaacctct tggtgaacct tagtacctaa aaggaaatct |
| 2041 | caccccatcc cacaccctgg aggatttcat ctcttgtata tgatgatctg gatccaccaa |
| 2101 | gacttgtttt atgctcaggg tcaatttctt ttttcttttt tttttttttt tttctttttc |
| 2161 | tttgagactg ggtctcgctt tgttgcccag gctggagtgg agtggcgtga tcttggctta |
| 2221 | ctgcagcctt tgcctccccg gctcgagcag tcctgcctca gcctccggag tagctgggac |
| 2281 | cacaggttca tgccaccatg gccagccaac ttttgcatgt tttgtagaga tggggtctca |
| 2341 | cagtgttgcc caggctggtc tcaaactcct gggctcaggc gatccacctg tctcagcctc |
| 2401 | ccagagtgct gggattacaa ttgtgagcca ccacgtccag ctggaagggt caacatcttt |
| 2461 | tacattctgc aagcacatct gcattttcac cccacccttc ccctccttct ccctttttat |
| 2521 | atcccatttt tatatcgatc tcttatttta caataaaact ttgctgccac ctgtgtgtct |
| 2581 | gaggggtg |

SEQ ID NO: 18 Human TP53 isoform h Amino Acid Sequence (NP_001263624.1)

| | |
|---|---|
| 1 | mddlmlspdd ieqwftedpg pdeaprmpea appvapapaa ptpaapapap swplsssvps |
| 61 | qktyqgsygf rlgflhsgta ksvtctyspa lnkmfcqlak tcpvqlwvds tpppgtrvra |
| 121 | maiykqsqhm tevvrrcphh ercsdsdgla ppqhlirveg nlrveylddr ntfrhsvvvp |
| 181 | yeppevgsdc ttihynymcn sscmggmnrr piltiitled ssgnllgrns fevrvcacpg |
| 241 | rdrrteeenl rkkgephhel ppgstkralp nntssspqpk kkpldgeyft lqmlldlrwc |
| 301 | yflinss |

SEQ ID NO: 19 Human TP53 transcript variant 4 cDNA Sequence
(NM_001276695.1; CDS: 320-1243)

| | |
|---|---|
| 1 | gatgggattg gggttttccc ctcccatgtg ctcaagactg gcgctaaaag ttttgagctt |
| 61 | ctcaaaagtc tagagccacc gtccagggag caggtagctg ctgggctccg gggacacttt |
| 121 | gcgttcgggc tgggagcgtg ctttccacga cggtgacacg cttccctgga ttggcagcca |
| 181 | gactgccttc cgggtcactg ccatggagga gccgcagtca gatcctagcg tcgagccccc |
| 241 | tctgagtcag gaaacatttt cagacctatg gaaactactt cctgaaaaca acgttctgtc |
| 301 | ccccttgccg tcccaagcaa tggatgattt gatgctgtcc ccggacgata ttgaacaatg |
| 361 | gttcactgaa gacccaggtc cagatgaagc tcccagaatg ccagaggctg ctccccccgt |
| 421 | ggcccctgca ccagcagctc ctacaccggc ggcccctgca ccagccccct cctggcccct |
| 481 | gtcatcttct gtcccttccc agaaaaccta ccagggcagc tacggtttcc gtctgggctt |
| 541 | cttgcattct gggacagcca agtctgtgac ttgcacgtac tcccctgccc tcaacaagat |
| 601 | gttttgccaa ctggccaaga cctgccctgt gcagctgtgg gttgattcca cacccccgcc |
| 661 | cggcacccgc gtccgcgcca tggccatcta caagcagtca cagcacatga cggaggttgt |
| 721 | gaggcgctgc ccccaccatg agcgctgctc agatagcgat ggtctggccc ctcctcagca |
| 781 | tcttatccga gtggaaggaa atttgcgtgt ggagtatttg gatgacagaa acacttttcg |
| 841 | acatagtgtg gtggtgccct atgagccgcc tgaggttggc tctgactgta ccaccatcca |
| 901 | ctacaactac atgtgtaaca gttcctgcat gggcggcatg aaccggaggc ccatcctcac |
| 961 | catcatcaca ctggaagact ccagtggtaa tctactggga cggaacagct ttgaggtgcg |
| 1021 | tgtttgtgcc tgtcctggga gagaccggcg cacagaggaa gagaatctcc gcaagaaagg |
| 1081 | ggagcctcac cacgagctgc ccccagggag cactaagcga gcactgccca acaacaccag |
| 1141 | ctcctctccc cagccaaaga agaaaccact ggatggagaa tatttcaccc ttcagatgct |
| 1201 | acttgactta cgatggtgtt acttcctgat aaactcgtcg taagttgaaa atattatccg |
| 1261 | tgggcgtgag cgcttcgaga tgttccgaga gctgaatgag gccttggaac tcaaggatgc |
| 1321 | ccaggctggg aaggagccag gggggagcag ggctcactcc agccacctga agtccaaaaa |
| 1381 | gggtcagtct acctcccgcc ataaaaaact catgttcaag acagaagggc ctgactcaga |
| 1441 | ctgacattct ccacttcttg ttccccactg acagcctccc acccccatct ctccctcccc |
| 1501 | tgccattttg ggttttgggt ctttgaaccc ttgcttgcaa taggtgtgcg tcagaagcac |
| 1561 | ccaggacttc catttgcttt gtcccggggc tccactgaac aagttggcct gcactggtgt |
| 1621 | tttgttgtgg ggaggaggat ggggagtagg acataccagc ttagatttta aggtttttac |
| 1681 | tgtgagggat gtttgggaga tgtaagaaat gttcttgcag ttaagggtta gtttacaatc |
| 1741 | agccacattc taggtagggg cccacttcac cgtactaacc agggaagctg tccctcactg |
| 1801 | ttgaattttc tctaacttca aggcccatat ctgtgaaatg ctggcatttg cacctacctc |
| 1861 | acagagtgca ttgtgagggt taatgaaata atgtacatct ggccttgaaa ccacctttta |
| 1921 | ttacatgggg tctagaactt gacccccttg agggtgcttg ttccctctcc ctgttggtcg |
| 1981 | gtgggttggt agtttctaca gttgggcagc tggttaggta gagggagttg tcaagtctct |
| 2041 | gctggcccag ccaaaccctg tctgacaacc tcttggtgaa ccttagtacc taaaaggaaa |
| 2101 | tctcacccca tcccacaccc tggaggattt catctcttgt atatgatgat ctggatccac |
| 2161 | caagacttgt tttatgctca gggtcaattt cttttttctt tttttttttt ttttttcttt |
| 2221 | ttctttgaga ctgggtctcg ctttgttgcc caggctggag tggagtggcg tgatcttggc |
| 2281 | ttactgcagc ctttgcctcc ccggctcgag cagtcctgcc tcagcctccg gagtagctgg |
| 2341 | gaccacaggt tcatgccacc atggccagcc aacttttgca tgttttgtag agatggggtc |
| 2401 | tcacagtgtt gcccaggctg gtctcaaact cctgggctca ggcgatccac ctgtctcagc |
| 2461 | ctcccagagt gctgggatta caattgtgag ccaccacgtc cagctggaag ggtcaacatc |
| 2521 | ttttacattc tgcaagcaca tctgcatttt caccccaccc ttcccctcct tctccctttt |
| 2581 | tatatcccat ttttatatcg atctcttatt ttacaataaa actttgctgc cacctgtgtg |
| 2641 | tctgaggggt g |

SEQ ID NO: 20 Human TP53 isoform i Amino Acid Sequence (NP_001263625.1)

| | |
|---|---|
| 1 | mddlmlspdd ieqwftedpg pdeaprmpea appvapapaa ptpaapapap swplsssvps |
| 61 | qktyqgsygf rlgflhsgta ksvtctyspa lnkmfcqlak tcpvqlwvds tpppgtrvra |
| 121 | maiykqsqhm tevvrrcphh ercsdsdgla ppqhlirveg nlrveylddr ntfrhsvvvp |
| 181 | yeppevgsdc ttihynymcn sscmggmnrr piltiitled ssgnllgrns fevrvcacpg |
| 241 | rdrrteeenl rkkgephhel ppgstkralp nntssspqpk kkpldgeyft lqdqtsfqke |
| 301 | nc |

SEQ ID NO: 21 Human TP53 transcript variant 3 cDNA sequence
(NM_001276696.1; CDS: 320-1228)

| | |
|---|---|
| 1 | gatgggattg gggttttccc ctcccatgtg ctcaagactg gcgctaaaag ttttgagctt |
| 61 | ctcaaaagtc tagagccacc gtccagggag caggtagctg ctgggctccg gggacacttt |
| 121 | gcgttcgggc tgggagcgtg ctttccacga cggtgacacg cttccctgga ttggcagcca |
| 181 | gactgccttc cgggtcactg ccatggagga gccgcagtca gatcctagcg tcgagccccc |
| 241 | tctgagtcag gaaacatttt cagacctatg gaaactactt cctgaaaaca acgttctgtc |
| 301 | ccccttgccg tcccaagcaa tggatgattt gatgctgtcc ccggacgata ttgaacaatg |
| 361 | gttcactgaa gacccaggtc cagatgaagc tcccagaatg ccagaggctg ctccccccgt |
| 421 | ggcccctgca ccagcagctc ctacaccggc ggcccctgca ccagccccct cctggcccct |
| 481 | gtcatcttct gtcccttccc agaaaaccta ccagggcagc tacggtttcc gtctgggctt |
| 541 | cttgcattct gggacagcca agtctgtgac ttgcacgtac tcccctgccc tcaacaagat |
| 601 | gttttgccaa ctggccaaga cctgccctgt gcagctgtgg gttgattcca cacccccgcc |
| 661 | cggcacccgc gtccgcgcca tggccatcta caagcagtca cagcacatga cggaggttgt |
| 721 | gaggcgctgc ccccaccatg agcgctgctc agatagcgat ggtctggccc ctcctcagca |
| 781 | tcttatccga gtggaaggaa atttgcgtgt ggagtatttg gatgacagaa acacttttcg |
| 841 | acatagtgtg gtggtgccct atgagccgcc tgaggttggc tctgactgta ccaccatcca |
| 901 | ctacaactac atgtgtaaca gttcctgcat gggcggcatg aaccggaggc ccatcctcac |
| 961 | catcatcaca ctggaagact ccagtggtaa tctactggga cggaacagct ttgaggtgcg |
| 1021 | tgtttgtgcc tgtcctggga gagaccggcg cacagaggaa gagaatctcc gcaagaaagg |
| 1081 | ggagcctcac cacgagctgc ccccagggag cactaagcga gcactgccca acaacaccag |
| 1141 | ctcctctccc cagccaaaga agaaaccact ggatggagaa tatttcaccc ttcaggacca |
| 1201 | gaccagcttt caaaaagaaa attgttaaag agagcatgaa aatggttcta tgactttgcc |
| 1261 | tgatacagat gctacttgac ttacgatggt gttacttcct gataaactcg tcgtaagttg |
| 1321 | aaaatattat ccgtgggcgt gagcgcttcg agatgttccg agagctgaat gaggccttgg |
| 1381 | aactcaagga tgcccaggct gggaaggagc caggggggag cagggctcac tccagccacc |
| 1441 | tgaagtccaa aaagggtcag tctacctccc gccataaaaa actcatgttc aagacagaag |
| 1501 | ggcctgactc agactgacat tctccacttc ttgttcccca ctgacagcct cccaccccca |
| 1561 | tctctccctc ccctgccatt ttgggttttg ggtctttgaa cccttgcttg caataggtgt |
| 1621 | gcgtcagaag cacccaggac ttccatttgc tttgtcccgg ggctccactg aacaagttgg |
| 1681 | cctgcactgg tgttttgttg tggggaggag gatggggagt aggacatacc agcttagatt |
| 1741 | ttaaggtttt tactgtgagg gatgtttggg agatgtaaga aatgttcttg cagttaaggg |
| 1801 | ttagtttaca atcagccaca ttctaggtag gggcccactt caccgtacta accagggaag |
| 1861 | ctgtccctca ctgttgaatt ttctctaact tcaaggccca tatctgtgaa atgctggcat |
| 1921 | ttgcacctac ctcacagagt gcattgtgag ggttaatgaa ataatgtaca tctggccttg |
| 1981 | aaaccacctt ttattacatg gggtctagaa cttgaccccc ttgagggtgc ttgttccctc |
| 2041 | tccctgttgg tcggtgggtt ggtagtttct acagttgggc agctggttag gtagagggag |
| 2101 | ttgtcaagtc tctgctggcc cagccaaacc ctgtctgaca acctcttggt gaaccttagt |
| 2161 | acctaaaagg aaatctcacc ccatcccaca ccctggagga tttcatctct tgtatatgat |
| 2221 | gatctggatc caccaagact tgttttatgc tcagggtcaa tttctttttt cttttttttt |
| 2281 | tttttttttc tttttctttg agactgggtc tcgctttgtt gcccaggctg gagtggagtg |
| 2341 | gcgtgatctt ggcttactgc agcctttgcc tccccggctc gagcagtcct gcctcagcct |
| 2401 | ccggagtagc tgggaccaca ggttcatgcc accatggcca gccaactttt gcatgttttg |
| 2461 | tagagatggg gtctcacagt gttgcccagg ctggtctcaa actcctgggc tcaggcgatc |
| 2521 | cacctgtctc agcctcccag agtgctggga ttacaattgt gagccaccac gtccagctgg |
| 2581 | aagggtcaac atcttttaca ttctgcaagc acatctgcat tttcacccca cccttcccct |
| 2641 | ccttctccct ttttatatcc catttttata tcgatctctt attttacaat aaaactttgc |
| 2701 | tgccacctgt gtgtctgagg ggtg |

SEQ ID NO: 22 Human TP53 isoform i Amino Acid Sequence (NP_001263626.1)

| | |
|---|---|
| 1 | maiykqsqhm tevvrrcphh ercsdsdgla ppqhlirveg nlrveylddr ntfrhsvvvp |
| 61 | yeppevgsdc ttihynymcn sscmggmnrr piltiitled ssgnllgrns fevrvcacpg |
| 121 | rdrrteeenl rkkgephhel ppgstkralp nntssspqpk kkpldgeyft lqirgrerfe |
| 181 | mfrelneale lkdaqagkep ggsrahsshl kskkgqstsr hkklmfkteg pdsd |

SEQ ID NO: 23 Human TP53 transcript variant 5 cDNA sequence
(NM_001276697.1; CDS: 360-1064)

| | |
|---|---|
| 1 | tgaggccagg agatggaggc tgcagtgagc tgtgatcaca ccactgtgct ccagcctgag |
| 61 | tgacagagca agaccctatc tcaaaaaaaa aaaaaaaaaa gaaaagctcc tgaggtgtag |
| 121 | acgccaactc tctctagctc gctagtgggt tgcaggaggt gcttacgcat gtttgtttct |
| 181 | ttgctgccgt cttccagttg ctttatctgt tcacttgtgc cctgactttc aactctgtct |
| 241 | ccttcctctt cctacagtac tcccctgccc tcaacaagat gttttgccaa ctggccaaga |
| 301 | cctgccctgt gcagctgtgg gttgattcca cacccccgcc cggcacccgc gtccgcgcca |
| 361 | tggccatcta caagcagtca cagcacatga cggaggttgt gaggcgctgc ccccaccatg |
| 421 | agcgctgctc agatagcgat ggtctggccc ctcctcagca tcttatccga gtggaaggaa |
| 481 | atttgcgtgt ggagtatttg gatgacagaa acacttttcg acatagtgtg gtggtgccct |
| 541 | atgagccgcc tgaggttggc tctgactgta ccaccatcca ctacaactac atgtgtaaca |
| 601 | gttcctgcat gggcggcatg aaccggaggc ccatcctcac catcatcaca ctggaagact |
| 661 | ccagtggtaa tctactggga cggaacagct ttgaggtgcg tgtttgtgcc tgtcctggga |
| 721 | gagaccggcg cacagaggaa gagaatctcc gcaagaaagg ggagcctcac cacgagctgc |
| 781 | ccccagggag cactaagcga gcactgccca acaacaccag ctcctctccc cagccaaaga |
| 841 | agaaaccact ggatggagaa tatttcaccc ttcagatccg tgggcgtgag cgcttcgaga |
| 901 | tgttccgaga gctgaatgag gccttggaac tcaaggatgc ccaggctggg aaggagccag |
| 961 | gggggagcag ggctcactcc agccacctga agtccaaaaa gggtcagtct acctcccgcc |
| 1021 | ataaaaaact catgttcaag acagaagggc ctgactcaga ctgacattct ccacttcttg |
| 1081 | ttccccactg acagcctccc acccccatct ctccctcccc tgccattttg ggttttgggt |
| 1141 | ctttgaaccc ttgcttgcaa taggtgtgcg tcagaagcac ccaggacttc catttgcttt |
| 1201 | gtcccggggc tccactgaac aagttggcct gcactggtgt tttgttgtgg ggaggaggat |
| 1261 | ggggagtagg acataccagc ttagatttta aggtttttac tgtgagggat gtttgggaga |
| 1321 | tgtaagaaat gttcttgcag ttaagggtta gtttacaatc agccacattc taggtagggg |
| 1381 | cccacttcac cgtactaacc agggaagctg tccctcactg ttgaattttc tctaacttca |
| 1441 | aggcccatat ctgtgaaatg ctggcatttg cacctacctc acagagtgca ttgtgagggt |
| 1501 | taatgaaata atgtacatct ggccttgaaa ccacctttta ttacatgggg tctagaactt |
| 1561 | gacccccttg agggtgcttg ttccctctcc ctgttggtcg gtgggttggt agtttctaca |
| 1621 | gttgggcagc tggttaggta gagggagttg tcaagtctct gctggcccag ccaaaccctg |
| 1681 | tctgacaacc tcttggtgaa ccttagtacc taaaaggaaa tctcacccca tcccacaccc |
| 1741 | tggaggattt catctcttgt atatgatgat ctggatccac caagacttgt tttatgctca |
| 1801 | gggtcaattt cttttttctt tttttttttt ttttttcttt ttctttgaga ctgggtctcg |
| 1861 | ctttgttgcc caggctggag tggagtggcg tgatcttggc ttactgcagc ctttgcctcc |
| 1921 | ccggctcgag cagtcctgcc tcagcctccg gagtagctgg gaccacaggt tcatgccacc |
| 1981 | atggccagcc aacttttgca tgttttgtag agatggggtc tcacagtgtt gcccaggctg |
| 2041 | gtctcaaact cctgggctca ggcgatccac ctgtctcagc ctcccagagt gctgggatta |
| 2101 | caattgtgag ccaccacgtc cagctggaag ggtcaacatc ttttacattc tgcaagcaca |
| 2161 | tctgcatttt caccccaccc ttcccctcct tctccctttt tatatcccat ttttatatcg |
| 2221 | atctcttatt ttacaataaa actttgctgc cacctgtgtg tctgaggggt g |

SEQ ID NO: 24 Human TP53 isoform k Amino Acid Sequence (NP_001263627.1)

| | |
|---|---|
| 1 | maiykqsqhm tevvrrcphh ercsdsdgla ppqhlirveg nlrveylddr ntfrhsvvvp |
| 61 | yeppevgsdc ttihynymcn sscmggmnrr piltiitled ssgnllgrns fevrvcacpg |
| 121 | rdrrteeenl rkkgephhel ppgstkralp nntssspqpk kkpldgeyft lqdqtsfqke |
| 181 | nc |

SEQ ID NO: 25 Human TP53 transcript variant 6 cDNA sequence
(NM_001276698.1; CDS: 360-908)

| | |
|---|---|
| 1 | tgaggccagg agatggaggc tgcagtgagc tgtgatcaca ccactgtgct ccagcctgag |
| 61 | tgacagagca agaccctatc tcaaaaaaaa aaaaaaaaaa gaaaagctcc tgaggtgtag |
| 121 | acgccaactc tctctagctc gctagtgggt tgcaggaggt gcttacgcat gtttgtttct |
| 181 | ttgctgccgt cttccagttg ctttatctgt tcacttgtgc cctgactttc aactctgtct |
| 241 | ccttcctctt cctacagtac tcccctgccc tcaacaagat gttttgccaa ctggccaaga |
| 301 | cctgccctgt gcagctgtgg gttgattcca cacccccgcc cggcacccgc gtccgcgcca |
| 361 | tggccatcta caagcagtca cagcacatga cggaggttgt gaggcgctgc ccccaccatg |
| 421 | agcgctgctc agatagcgat ggtctggccc ctcctcagca tcttatccga gtggaaggaa |
| 481 | atttgcgtgt ggagtatttg gatgacagaa acacttttcg acatagtgtg gtggtgccct |
| 541 | atgagccgcc tgaggttggc tctgactgta ccaccatcca ctacaactac atgtgtaaca |
| 601 | gttcctgcat gggcggcatg aaccggaggc ccatcctcac catcatcaca ctggaagact |
| 661 | ccagtggtaa tctactggga cggaacagct ttgaggtgcg tgtttgtgcc tgtcctggga |
| 721 | gagaccggcg cacagaggaa gagaatctcc gcaagaaagg ggagcctcac cacgagctgc |
| 781 | ccccagggag cactaagcga gcactgccca acaacaccag ctcctctccc cagccaaaga |
| 841 | agaaaccact ggatggagaa tatttcaccc ttcaggacca gaccagcttt caaaaagaaa |
| 901 | attgttaaag agagcatgaa aatggttcta tgactttgcc tgatacagat gctacttgac |
| 961 | ttacgatggt gttacttcct gataaactcg tcgtaagttg aaaatattat ccgtgggcgt |
| 1021 | gagcgcttcg agatgttccg agagctgaat gaggccttgg aactcaagga tgcccaggct |
| 1081 | gggaaggagc caggggggag cagggctcac tccagccacc tgaagtccaa aaagggtcag |
| 1141 | tctacctccc gccataaaaa actcatgttc aagacagaag ggcctgactc agactgacat |
| 1201 | tctccacttc ttgttcccca ctgacagcct cccaccccca tctctccctc ccctgccatt |
| 1261 | ttgggttttg ggtctttgaa cccttgcttg caataggtgt gcgtcagaag cacccaggac |
| 1321 | ttccatttgc tttgtcccgg ggctccactg aacaagttgg cctgcactgg tgttttgttg |
| 1381 | tggggaggag gatggggagt aggacatacc agcttagatt ttaaggtttt tactgtgagg |
| 1441 | gatgtttggg agatgtaaga aatgttcttg cagttaaggg ttagtttaca atcagccaca |
| 1501 | ttctaggtag gggcccactt caccgtacta accagggaag ctgtccctca ctgttgaatt |
| 1561 | ttctctaact tcaaggccca tatctgtgaa atgctggcat ttgcacctac ctcacagagt |
| 1621 | gcattgtgag ggttaatgaa ataatgtaca tctggccttg aaaccacctt ttattacatg |
| 1681 | gggtctagaa cttgaccccc ttgagggtgc ttgttccctc tccctgttgg tcggtgggtt |
| 1741 | ggtagtttct acagttgggc agctggttag gtagagggag ttgtcaagtc tctgctggcc |
| 1801 | cagccaaacc ctgtctgaca acctcttggt gaaccttagt acctaaaagg aaatctcacc |
| 1861 | ccatcccaca ccctggagga tttcatctct tgtatatgat gatctggatc caccaagact |
| 1921 | tgttttatgc tcagggtcaa tttctttttt cttttttttt tttttttttc tttttctttg |
| 1981 | agactgggtc tcgctttgtt gcccaggctg gagtggagtg gcgtgatctt ggcttactgc |
| 2041 | agcctttgcc tccccggctc gagcagtcct gcctcagcct ccggagtagc tgggaccaca |
| 2101 | ggttcatgcc accatggcca gccaactttt gcatgttttg tagagatggg gtctcacagt |
| 2161 | gttgcccagg ctggtctcaa actcctgggc tcaggcgatc cacctgtctc agcctcccag |
| 2221 | agtgctggga ttacaattgt gagccaccac gtccagctgg aagggtcaac atcttttaca |
| 2281 | ttctgcaagc acatctgcat tttcacccca cccttcccct ccttctccct ttttatatcc |
| 2341 | catttttata tcgatctctt attttacaat aaaactttgc tgccacctgt gtgtctgagg |
| 2401 | ggtg |

SEQ ID NO: 26 Human TP53 isoform 1 Amino Acid Sequence (NP_001263628.1)

| | |
|---|---|
| 1 | maiykqsqhm tevvrrcphh ercsdsdgla ppqhlirveg nlrveylddr ntfrhsvvvp |
| 61 | yeppevgsdc ttihynymcn sscmggmnrr piltiitled ssgnllgrns fevrvcacpg |
| 121 | rdrrteeenl rkkgephhel ppgstkralp nntssspqpk kkpldgeyft lqmlldlrwc |
| 181 | yflinss |

SEQ ID NO: 27 Human TP53 transcript variant 7 cDNA sequence
(NM_001276699.1; CDS: 360-923)

| | |
|---|---|
| 1 | tgaggccagg agatggaggc tgcagtgagc tgtgatcaca ccactgtgct ccagcctgag |
| 61 | tgacagagca agaccctatc tcaaaaaaaa aaaaaaaaaa gaaaagctcc tgaggtgtag |
| 121 | acgccaactc tctctagctc gctagtgggt tgcaggaggt gcttacgcat gtttgtttct |
| 181 | ttgctgccgt cttccagttg ctttatctgt tcacttgtgc cctgactttc aactctgtct |
| 241 | ccttcctctt cctacagtac tcccctgccc tcaacaagat gttttgccaa ctggccaaga |
| 301 | cctgccctgt gcagctgtgg gttgattcca cacccccgcc cggcacccgc gtccgcgcca |
| 361 | tggccatcta caagcagtca cagcacatga cggaggttgt gaggcgctgc ccccaccatg |
| 421 | agcgctgctc agatagcgat ggtctggccc ctcctcagca tcttatccga gtggaaggaa |
| 481 | atttgcgtgt ggagtatttg gatgacagaa acacttttcg acatagtgtg gtggtgccct |
| 541 | atgagccgcc tgaggttggc tctgactgta ccaccatcca ctacaactac atgtgtaaca |
| 601 | gttcctgcat gggcggcatg aaccggaggc ccatcctcac catcatcaca ctggaagact |
| 661 | ccagtggtaa tctactggga cggaacagct ttgaggtgcg tgtttgtgcc tgtcctggga |
| 721 | gagaccggcg cacagaggaa gagaatctcc gcaagaaagg ggagcctcac cacgagctgc |
| 781 | ccccagggag cactaagcga gcactgccca acaacaccag ctcctctccc cagccaaaga |
| 841 | agaaaccact ggatggagaa tatttcaccc ttcagatgct acttgactta cgatggtgtt |
| 901 | acttcctgat aaactcgtcg taagttgaaa atattatccg tgggcgtgag cgcttcgaga |
| 961 | tgttccgaga gctgaatgag gccttggaac tcaaggatgc ccaggctggg aaggagccag |
| 1021 | gggggagcag ggctcactcc agccacctga agtccaaaaa gggtcagtct acctcccgcc |
| 1081 | ataaaaaact catgttcaag acagaagggc ctgactcaga ctgacattct ccacttcttg |
| 1141 | ttccccactg acagcctccc acccccatct ctccctcccc tgccattttg ggttttgggt |
| 1201 | ctttgaaccc ttgcttgcaa taggtgtgcg tcagaagcac ccaggacttc catttgcttt |
| 1261 | gtcccggggc tccactgaac aagttggcct gcactggtgt tttgttgtgg ggaggaggat |
| 1321 | ggggagtagg acataccagc ttagatttta aggtttttac tgtgagggat gtttgggaga |
| 1381 | tgtaagaaat gttcttgcag ttaagggtta gtttacaatc agccacattc taggtagggg |
| 1441 | cccacttcac cgtactaacc agggaagctg tccctcactg ttgaattttc tctaacttca |
| 1501 | aggcccatat ctgtgaaatg ctggcatttg cacctacctc acagagtgca ttgtgagggt |
| 1561 | taatgaaata atgtacatct ggccttgaaa ccacctttta ttacatgggg tctagaactt |
| 1621 | gacccccttg agggtgcttg ttccctctcc ctgttggtcg gtgggttggt agtttctaca |
| 1681 | gttgggcagc tggttaggta gagggagttg tcaagtctct gctggcccag ccaaaccctg |
| 1741 | tctgacaacc tcttggtgaa ccttagtacc taaaaggaaa tctcacccca tcccacaccc |
| 1801 | tggaggattt catctcttgt atatgatgat ctggatccac caagacttgt tttatgctca |
| 1861 | gggtcaattt cttttttctt tttttttttt ttttttcttt ttctttgaga ctgggtctcg |
| 1921 | ctttgttgcc caggctggag tggagtggcg tgatcttggc ttactgcagc ctttgcctcc |
| 1981 | ccggctcgag cagtcctgcc tcagcctccg gagtagctgg gaccacaggt tcatgccacc |
| 2041 | atggccagcc aacttttgca tgttttgtag agatggggtc tcacagtgtt gcccaggctg |
| 2101 | gtctcaaact cctgggctca ggcgatccac ctgtctcagc ctcccagagt gctgggatta |
| 2161 | caattgtgag ccaccacgtc cagctggaag ggtcaacatc ttttacattc tgcaagcaca |
| 2221 | tctgcatttt caccccaccc ttcccctcct tctccctttt tatatcccat ttttatatcg |
| 2281 | atctcttatt ttacaataaa actttgctgc cacctgtgtg tctgaggggt g |

SEQ ID NO: 28 Mouse TP53 isoform b Amino Acid Sequence (NP_001120705.1)

| | |
|---|---|
| 1 | mtameesqsd islelplsqe tfsglwkllp pedilpsphc mddlllpqdv eeffegpsea |
| 61 | lrvsgapaaq dpvtetpgpv apapatpwpl ssfvpsqkty qgnygfhlgf lqsgtaksvm |
| 121 | ctyspplnkl fcqlaktcpv qlwvsatppa gsrvramaiy kksqhmtevv rrcphhercs |
| 181 | dgdglappqh lirvegnlyp eyledrqtfr hsvvvpyepp eagseyttih ykymcnsscm |
| 241 | ggmnrrpilt iitledssgn llgrdsfevr vcacpgrdrr teeenfrkke vlcpelppgs |
| 301 | akralptcts asppqkkkpl dgeyftlkir grkrfemfre inealelkda hateesgdsr |
| 361 | ahsslqpraf qalikeespn c |

SEQ ID NO: 29 Mouse TP53 transcript variant 2 cDNA sequence
(NM_001127233.1; CDS: 158-1303)

| | |
|---|---|
| 1 | tttcccctcc cacgtgctca ccctggctaa agttctgtag cttcagttca ttgggaccat |
| 61 | cctggctgta ggtagcgact acagttaggg ggcacctagc attcaggccc tcatcctcct |
| 121 | ccttcccagc agggtgtcac gcttctccga agactggatg actgccatgg aggagtcaca |
| 181 | gtcggatatc agcctcgagc tccctctgag ccaggagaca ttttcaggct tatggaaact |
| 241 | acttcctcca gaagatatcc tgccatcacc tcactgcatg gacgatctgt tgctgcccca |
| 301 | ggatgttgag gagttttttg aaggcccaag tgaagccctc cgagtgtcag gagctcctgc |
| 361 | agcacaggac cctgtcaccg agacccctgg gccagtggcc cctgccccag ccactccatg |
| 421 | gcccctgtca tcttttgtcc cttctcaaaa aacttaccag ggcaactatg gcttccacct |
| 481 | gggcttcctg cagtctggga cagccaagtc tgttatgtgc acgtactctc ctcccctcaa |
| 541 | taagctattc tgccagctgg cgaagacgtg ccctgtgcag ttgtgggtca gcgccacacc |
| 601 | tccagctggg agccgtgtcc gcgccatggc catctacaag aagtcacagc acatgacgga |
| 661 | ggtcgtgaga cgctgccccc accatgagcg ctgctccgat ggtgatggcc tggctcctcc |
| 721 | ccagcatctt atccgggtgg aaggaaattt gtatcccgag tatctggaag acaggcagac |
| 781 | ttttcgccac agcgtggtgg taccttatga gccacccgag gccggctctg agtataccac |
| 841 | catccactac aagtacatgt gtaatagctc ctgcatgggg ggcatgaacc gccgacctat |
| 901 | ccttaccatc atcacactgg aagactccag tgggaacctt ctgggacggg acagctttga |
| 961 | ggttcgtgtt tgtgcctgcc ctgggagaga ccgccgtaca gaagaagaaa atttccgcaa |
| 1021 | aaaggaagtc ctttgccctg aactgccccc agggagcgca aagagagcgc tgcccacctg |
| 1081 | cacaagcgcc tctcccccgc aaaagaaaaa accacttgat ggagagtatt tcaccctcaa |
| 1141 | gatccgcggg cgtaaacgct tcgagatgtt ccgggagctg aatgaggcct tagagttaaa |
| 1201 | ggatgcccat gctacagagg agtctggaga cagcagggct cactccagcc tccagcctag |
| 1261 | agccttccaa gccttgatca aggaggaaag cccaaactgc tagctcccat cacttcatcc |
| 1321 | ctcccctttt ctgtcttcct atagctacct gaagaccaag aagggccagt ctacttcccg |
| 1381 | ccataaaaaa acaatggtca agaaagtggg gcctgactca gactgactgc ctctgcatcc |
| 1441 | cgtccccatc accagcctcc ccctctcctt gctgtcttat gacttcaggg ctgagacaca |
| 1501 | atcctcccgg tcccttctgc tgcctttttt accttgtagc tagggctcag ccccctctct |
| 1561 | gagtagtggt tcctggccca agttggggaa taggttgata gttgtcaggt ctctgctggc |
| 1621 | ccagcgaaat tctatccagc cagttgttgg accctggcac ctacaatgaa atctcaccct |
| 1681 | accccacacc ctgtaagatt ctatcttggg ccctcatagg gtccatatcc tccagggcct |
| 1741 | actttccttc cattctgcaa agcctgtctg catttatcca ccccccaccc tgtctccctc |
| 1801 | tttttttttt ttttacccct ttttatatat caatttccta ttttacaata aaattttgtt |
| 1861 | atcacttaaa aaaaaaa |

SEQ ID NO: 30 Mouse TP53 isoform a Amino Acid Sequence (NP_035770.2)

| | |
|---|---|
| 1 | mtameesqsd islelplsqe tfsglwkllp pedilpsphc mddlllpqdv eeffegpsea |
| 61 | lrvsgapaaq dpvtetpgpv apapatpwpl ssfvpsqkty qgnygfhlgf lqsgtaksvm |
| 121 | ctyspplnkl fcqlaktcpv qlwvsatppa gsrvramaiy kksqhmtevv rrcphhercs |
| 181 | dgdglappqh lirvegnlyp eyledrqtfr hsvvvpyepp eagseyttih ykymcnsscm |
| 241 | ggmnrrpilt iitledssgn llgrdsfevr vcacpgrdrr teeenfrkke vlcpelppgs |
| 301 | akralptcts asppqkkkpl dgeyftlkir grkrfemfre lnealelkda hateesgdsr |
| 361 | ahssylktkk gqstsrhkkt mvkkvgpdsd |

SEQ ID NO: 31 Mouse TP53 transcript variant 1 cDNA sequence (NM_011640.3;
CDS: 158-1330)

| | |
|---|---|
| 1 | tttcccctcc cacgtgctca ccctggctaa agttctgtag cttcagttca ttgggaccat |
| 61 | cctggctgta ggtagcgact acagttaggg ggcacctagc attcaggccc tcatcctcct |
| 121 | ccttcccagc agggtgtcac gcttctccga agactggatg actgccatgg aggagtcaca |
| 181 | gtcggatatc agcctcgagc tccctctgag ccaggagaca ttttcaggct tatggaaact |
| 241 | acttcctcca gaagatatcc tgccatcacc tcactgcatg gacgatctgt tgctgcccca |
| 301 | ggatgttgag gagttttttg aaggcccaag tgaagccctc cgagtgtcag gagctcctgc |
| 361 | agcacaggac cctgtcaccg agacccctgg gccagtggcc cctgccccag ccactccatg |
| 421 | gcccctgtca tcttttgtcc cttctcaaaa aacttaccag ggcaactatg gcttccacct |
| 481 | gggcttcctg cagtctggga cagccaagtc tgttatgtgc acgtactctc ctcccctcaa |
| 541 | taagctattc tgccagctgg cgaagacgtg ccctgtgcag ttgtgggtca gcgccacacc |
| 601 | tccagctggg agccgtgtcc gcgccatggc catctacaag aagtcacagc acatgacgga |
| 661 | ggtcgtgaga cgctgccccc accatgagcg ctgctccgat ggtgatggcc tggctcctcc |
| 721 | ccagcatctt atccgggtgg aaggaaattt gtatcccgag tatctggaag acaggcagac |
| 781 | ttttcgccac agcgtggtgg taccttatga gccacccgag gccggctctg agtataccac |
| 841 | catccactac aagtacatgt gtaatagctc ctgcatgggg ggcatgaacc gccgacctat |
| 901 | ccttaccatc atcacactgg aagactccag tgggaacctt ctgggacggg acagctttga |
| 961 | ggttcgtgtt tgtgcctgcc ctgggagaga ccgccgtaca gaagaagaaa atttccgcaa |
| 1021 | aaaggaagtc ctttgccctg aactgccccc agggagcgca aagagagcgc tgcccacctg |
| 1081 | cacaagcgcc tctcccccgc aaaagaaaaa accacttgat ggagagtatt tcaccctcaa |
| 1141 | gatccgcggg cgtaaacgct tcgagatgtt ccgggagctg aatgaggcct tagagttaaa |
| 1201 | ggatgcccat gctacagagg agtctggaga cagcagggct cactccagct acctgaagac |
| 1261 | caagaagggc cagtctactt cccgccataa aaaaacaatg gtcaagaaag tggggcctga |
| 1321 | ctcagactga ctgcctctgc atcccgtccc catcaccagc ctccccctct ccttgctgtc |
| 1381 | ttatgacttc agggctgaga cacaatcctc ccggtccctt ctgctgcctt ttttaccttg |
| 1441 | tagctagggc tcagccccct ctctgagtag tggttcctgg cccaagttgg ggaataggtt |
| 1501 | gatagttgtc aggtctctgc tggcccagcg aaattctatc cagccagttg ttggaccctg |
| 1561 | gcacctacaa tgaaatctca ccctacccca caccctgtaa gattctatct tgggccctca |
| 1621 | tagggtccat atcctccagg gcctactttc cttccattct gcaaagcctg tctgcattta |
| 1681 | tccacccccc accctgtctc cctctttttt ttttttttac ccctttttat atatcaattt |
| 1741 | cctattttac aataaaattt tgttatcact taaaaaaaaa a |

SEQ ID NO: 32 Human RB1 Amino Acid Sequence (NP_000312.2)

| | |
|---|---|
| 1 | mppktprkta ataaaaaaep papppppppe edpeqdsgpe dlplvrlefe eteepdftal |
| 61 | cqklkipdhv rerawltwek vssvdgvlgg yiqkkkelwg icifiaavdl demsftftel |
| 121 | qknieisvhk ffnllkeidt stkvdnamsr llkkydvlfa lfsklertce liyltqpsss |
| 181 | isteinsalv lkvswitfll akgevlqmed dlvisfqlml cvldyfikls ppmllkepyk |
| 241 | tavipingsp rtprrgqnrs ariakqlend triievlcke hecnidevkn vyfknfipfm |
| 301 | nslglvtsng lpevenlskr yeeiylknkd ldarlfldhd ktlqtdsids fetqrtprks |
| 361 | nldeevnvip phtpvrtvmn tiqqlmmiln sasdqpsenl isyfnnctvn pkesilkrvk |
| 421 | digyifkekf akavgqgcve igsqryklgv rlyyrvmesm lkseeerlsi qnfskllndn |
| 481 | ifhmsllaca levvmatysr stsqnldsgt dlsfpwilnv lnlkafdfyk viesfikaeg |
| 541 | nltremikhl ercehrimes lawlsdsplf dlikqskdre gptdhlesac plnlplqnnh |
| 601 | taadmylspv rspkkkgstt rvnstanaet qatsafqtqk plkstslslf ykkvyrlayl |
| 661 | rlntlcerll sehpelehii wtlfqhtlqn eyelmrdrhl dqimmcsmyg ickvknidlk |
| 721 | fkiivtaykd lphavqetfk rvlikeeeyd siivfynsvf mqrlktnilq yastrpptls |
| 781 | piphiprspy kfpssplrip ggniyisplk spykiseglp tptkmtprsr ilvsigesfg |
| 841 | tsekfqkinq mvcnsdrvlk rsaegsnppk plkklrfdie gsdeadgskh lpgeskfqqk |
| 901 | laemtstrtr mqkqkmndsm dtsnkeek |

SEQ ID NO: 33 Human RB1 transcript DNA sequence (NM_000321.2;
CDS: 167 . . . 2953)

| | |
|---|---|
| 1 | gctcagttgc cgggcggggg agggcgcgtc cggtttttct caggggacgt tgaaattatt |
| 61 | tttgtaacgg gagtcgggag aggacggggc gtgccccgac gtgcgcgcgc gtcgtcctcc |
| 121 | ccggcgctcc tccacagctc gctggctccc gccgcggaaa ggcgtcatgc cgcccaaaac |
| 181 | cccccgaaaa acggccgcca ccgccgccgc tgccgccgcg gaacccccgg caccgccgcc |
| 241 | gccgccccct cctgaggagg acccagagca ggacagcggc ccggaggacc tgcctctcgt |
| 301 | caggcttgag tttgaagaaa cagaagaacc tgattttact gcattatgtc agaaattaaa |
| 361 | gataccagat catgtcagag agagagcttg gttaacttgg gagaaagttt catctgtgga |
| 421 | tggagtattg ggaggttata ttcaaaagaa aaaggaactg tggggaatct gtatctttat |
| 481 | tgcagcagtt gacctagatg agatgtcgtt cacttttact gagctacaga aaaacataga |
| 541 | aatcagtgtc cataaattct ttaacttact aaaagaaatt gataccagta ccaaagttga |
| 601 | taatgctatg tcaagactgt tgaagaagta tgatgtattg tttgcactct tcagcaaatt |
| 661 | ggaaaggaca tgtgaactta tatatttgac acaacccagc agttcgatat ctactgaaat |
| 721 | aaattctgca ttggtgctaa aagtttcttg gatcacattt ttattagcta aaggggaagt |
| 781 | attacaaatg gaagatgatc tggtgatttc atttcagtta atgctatgtg tccttgacta |
| 841 | ttttattaaa ctctcacctc ccatgttgct caaagaacca tataaaacag ctgttatacc |
| 901 | cattaatggt tcacctcgaa cacccaggcg aggtcagaac aggagtgcac ggatagcaaa |
| 961 | acaactagaa aatgatacaa gaattattga agttctctgt aaagaacatg aatgtaatat |
| 1021 | agatgaggtg aaaaatgttt atttcaaaaa ttttatacct tttatgaatt ctcttggact |
| 1081 | tgtaacatct aatggacttc cagaggttga aaatctttct aaacgatacg aagaaattta |
| 1141 | tcttaaaaat aaagatctag atgcaagatt atttttggat catgataaaa ctcttcagac |
| 1201 | tgattctata gacagttttg aaacacagag aacaccacga aaaagtaacc ttgatgaaga |
| 1261 | ggtgaatgta attcctccac acactccagt taggactgtt atgaacacta tccaacaatt |
| 1321 | aatgatgatt ttaaattcag caagtgatca accttcagaa aatctgattt cctattttaa |
| 1381 | caactgcaca gtgaatccaa aagaaagtat actgaaaaga gtgaaggata taggatacat |
| 1441 | ctttaaagag aaatttgcta aagctgtggg acagggttgt gtcgaaattg gatcacagcg |
| 1501 | atacaaactt ggagttcgct tgtattaccg agtaatggaa tccatgctta aatcagaaga |
| 1561 | agaacgatta tccattcaaa attttagcaa acttctgaat gacaacattt ttcatatgtc |
| 1621 | tttattggcg tgcgctcttg aggttgtaat ggccacatat agcagaagta catctcagaa |
| 1681 | tcttgattct ggaacagatt tgtctttccc atggattctg aatgtgctta atttaaaagc |
| 1741 | ctttgatttt tacaaagtga tcgaaagttt tatcaaagca gaaggcaact tgacaagaga |
| 1801 | aatgataaaa catttagaac gatgtgaaca tcgaatcatg gaatcccttg catggctctc |
| 1861 | agattcacct ttatttgatc ttattaaaca atcaaaggac cgagaaggac caactgatca |
| 1921 | ccttgaatct gcttgtcctc ttaatcttcc tctccagaat aatcacactg cagcagatat |
| 1981 | gtatctttct cctgtaagat ctccaaagaa aaaaggttca actacgcgtg taaattctac |
| 2041 | tgcaaatgca gagacacaag caacctcagc cttccagacc cagaagccat tgaaatctac |
| 2101 | ctctctttca ctgttttata aaaaagtgta tcggctagcc tatctccggc taaatacact |
| 2161 | ttgtgaacgc cttctgtctg agcacccaga attagaacat atcatctgga cccttttcca |
| 2221 | gcacaccctg cagaatgagt atgaactcat gagagacagg catttggacc aaattatgat |
| 2281 | gtgttccatg tatggcatat gcaaagtgaa gaatatagac cttaaattca aaatcattgt |
| 2341 | aacagcatac aaggatcttc ctcatgctgt tcaggagaca ttcaaacgtg ttttgatcaa |
| 2401 | agaagaggag tatgattcta ttatagtatt ctataactcg gtcttcatgc agagactgaa |
| 2461 | aacaaatatt ttgcagtatg cttccaccag gccccctacc ttgtcaccaa tacctcacat |
| 2521 | tcctcgaagc ccttacaagt ttcctagttc acccttacgg attcctggag ggaacatcta |
| 2581 | tatttcaccc ctgaagagtc catataaaat ttcagaaggt ctgccaacac caacaaaaat |
| 2641 | gactccaaga tcaagaatct tagtatcaat tggtgaatca ttcgggactt ctgagaagtt |
| 2701 | ccagaaaata aatcagatgg tatgtaacag cgaccgtgtg ctcaaaagaa gtgctgaagg |
| 2761 | aagcaaccct cctaaaccac tgaaaaaact acgctttgat attgaaggat cagatgaagc |
| 2821 | agatggaagt aaacatctcc caggagagtc caaatttcag cagaaactgg cagaaatgac |
| 2881 | ttctactcga acacgaatgc aaaagcagaa aatgaatgat agcatggata cctcaaacaa |
| 2941 | ggaagagaaa tgaggatctc aggaccttgg tggacactgt gtacacctct ggattcattg |
| 3001 | tctctcacag atgtgactgt ataactttcc caggttctgt ttatggccac atttaatatc |
| 3061 | ttcagctctt tttgtggata taaaatgtgc agatgcaatt gtttgggtga ttcctaagcc |
| 3121 | acttgaaatg ttagtcattg ttatttatac aagattgaaa atcttgtgta aatcctgcca |
| 3181 | tttaaaaagt tgtagcagat tgtttcctct tccaaagtaa aattgctgtg ctttatggat |
| 3241 | agtaagaatg gccctagagt gggagtcctg ataacccagg cctgtctgac tactttgcct |
| 3301 | tcttttgtag catataggtg atgtttgctc ttgtttttat taatttatat gtatattttt |
| 3361 | ttaatttaac atgaacaccc ttagaaaatg tgtcctatct atcttccaaa tgcaatttga |
| 3421 | ttgactgccc attcaccaaa attatcctga actcttctgc aaaaatggat attattagaa |
| 3481 | attagaaaaa aattactaat tttacacatt agattttatt ttactattgg aatctgatat |
| 3541 | actgtgtgct tgttttataa aattttgctt ttaattaaat aaaagctgga agcaaagtat |
| 3601 | aaccatatga tactatcata ctactgaaac agatttcata cctcagaatg taaaagaact |
| 3661 | tactgattat tttcttcatc caacttatgt ttttaaatga ggattattga tagtactctt |
| 3721 | ggtttttata ccattcagat cactgaattt ataaagtacc catctagtac ttgaaaaagt |
| 3781 | aaagtgttct gccagatctt aggtatagag gaccctaaca cagtatatcc caagtgcact |
| 3841 | ttctaatgtt tctgggtcct gaagaattaa gatacaaatt aattttactc cataaacaga |
| 3901 | ctgttaatta taggagcctt aatttttttt tcatagagat ttgtctaatt gcatctcaaa |
| 3961 | attattctgc cctccttaat ttgggaaggt ttgtgttttc tctggaatgg tacatgtctt |
| 4021 | ccatgtatct tttgaactgg caattgtcta tttatctttt atttttttaa gtcagtatgg |
| 4081 | tctaacactg gcatgttcaa agccacatta tttctagtcc aaaattacaa gtaatcaagg |
| 4141 | gtcattatgg gttaggcatt aatgtttcta tctgattttg tgcaaaagct tcaaattaaa |
| 4201 | acagctgcat tagaaaaaga ggcgcttctc ccctccccta cacctaaagg tgtatttaaa |
| 4261 | ctatcttgtg tgattaactt atttagagat gctgtaactt aaaatagggg atatttaagg |
| 4321 | tagcttcagc tagcttttag gaaaatcact ttgtctaact cagaattatt tttaaaaaga |
| 4381 | aatctggtct tgttagaaaa caaaatttta ttttgtgctc atttaagttt caaacttact |
| 4441 | attttgacag ttattttgat aacaatgaca ctagaaaact tgactccatt tcatcattgt |
| 4501 | ttctgcatga atatcataca aatcagttag tttttaggtc aagggcttac tatttctggg |
| 4561 | tcttttgcta ctaagttcac attagaatta gtgccagaat tttaggaact tcagagatcg |
| 4621 | tgtattgaga tttcttaaat aatgcttcag atattattgc tttattgctt ttttgtattg |
| 4681 | gttaaaactg tacatttaaa attgctatgt tactattttc tacaattaat agtttgtcta |
| 4741 | ttttaaaata aattagttgt taagagtctt aa |

SEQ ID NO: 34 Mouse RB1 Amino Acid Sequence ( )

| | |
|---|---|
| 1 | mppkaprraa aaeppppppp ppreddpaqd sgpeelplar lefeeieepe fialcqklkv |
| 61 | pdhvrerawl twekvssvdg ilegyiqkkk elwgicifia avdldempft ftelqksiet |
| 121 | svykffdllk eidtstkvdn amsrllkkyn vlcalyskle rtceliyltq pssalstein |
| 181 | smlvlkiswi tfllakgevl qmeddlvisf qlmlcvvdyf ikfsppallr epyktaaipi |
| 241 | ngsprtprrg qnrsariakq lendtriiev lckehecnid evknvyfknf ipfinslgiv |
| 301 | ssnglpeves lskryeevyl knkdldarlf ldhdktlqtd pidsfetert prknnpdeea |
| 361 | nvvtphtpvr tvmntiqqlm vilnsasdqp senlisyfnn ctvnpkenil krvkdvghif |
| 421 | kekfanavgq gcvdigvqry klgvrlyyrv mesmlkseee rlsiqnfskl lndnifhmsl |
| 481 | lacalevvma tysrstlqhl dsgtdlsfpw ilnvlnlkaf dfykviesfi kveanltrem |
| 541 | ikhlercehr imeslawlsd splfdlikqs kdgegpdnle pacplslplq gnhtaadmyl |
| 601 | splrspkkrt sttrvnsaan tetqaasafh tqkplkstsl alfykkvyrl aylrlntlca |
| 661 | rllsdhpele hiiwtlfqht lqneyelmrd rhldqimmcs mygickvkni dlkfkiivta |
| 721 | ykdlphaaqe tfkrvliree efdsiivfyn svfmqrlktn ilqyastrpp tlspiphipr |
| 781 | spykfssspl ripggniyis plkspykise glptptkmtp rsrilvsige sfgtsekfqk |
| 841 | inqmvcnsdr vlkrsaeggn ppkplkklrf diegadeadg skhlpaeskf qqklaemtst |
| 901 | rtrmqkqrmn eskdvsnkee k |

SEQ ID NO: 35 Mouse RB1 transcript DNA sequence (NM_009029.3; CDS:
182 . . . 2947)

| | |
|---|---|
| 1 | gttttcccgc ggttggcccg ggcgctcggt tgccgggcgc gtccggtttt cctcggggga |
| 61 | cgttcccatt atttttgtaa cgggagtcgg gtgaggacgg ggcgtgcccg gcgtgcgcgc |
| 121 | gcgcccgccc gcctccccgc gcgcctccct cggctgctcg cgccggcccg cgctgcgcgt |
| 181 | catgccgccc aaagccccgc gcagagccgc ggccgccgag cccccgccac cgccgccgcc |
| 241 | gccgcctcgg gaggacgacc ccgcgcagga cagcggcccc gaagagctgc ccctggccag |
| 301 | gcttgagttt gaagaaattg aagaacccga atttattgca ttatgtcaaa agttaaaggt |
| 361 | acccgatcat gtcagagaaa gagcttggct aacttgggag aaagtttcat ccgtggatgg |
| 421 | aatcctggaa ggatatattc aaaagaagaa ggaactctgg ggcatctgca tctttatcgc |
| 481 | agcagttgat ctagatgaga tgccattcac ttttactgag ctacagaaaa gcatagaaac |
| 541 | cagtgtctat aaattctttg acttattaaa agaaatcgat accagtacca aggttgataa |
| 601 | tgctatgtca agactattga agaagtataa tgtgttatgt gcactctaca gcaaattaga |
| 661 | acggacgtgt gaacttatat atttgacaca acccagcagt gcgttatcta ctgaaataaa |
| 721 | ttctatgttg gtgctaaaaa tttcttggat cactttttta ctagctaaag gagaagtatt |
| 781 | acagatggaa gatgacctgg taatctcatt tcagctaatg ttgtgtgtag ttgactattt |
| 841 | tattaagttc tcacctcctg cactactcag agagccatac aaaacagctg caatccccat |
| 901 | taatggttca cctcgaacac ccagaagagg tcagaacagg agcgctcgga tagcaaaaca |
| 961 | actagaaaat gatacgagga ttatcgaggt tctctgtaaa gaacacgagt gtaatataga |
| 1021 | tgaggtgaaa aatgtttatt tcaaaaattt tatccctttt ataaattcac ttggaattgt |
| 1081 | atcatctaat ggacttccag aggttgaaag tctttctaaa cgctatgaag aagtttatct |
| 1141 | taaaaacaaa gatttagatg caagactgtt tttggatcat gataaaacac ttcagactga |
| 1201 | tcctatagac agttttgaaa cagagagaac gccacgaaaa aacaaccctg atgaagaggc |
| 1261 | aaacgtggtt actccacaca ctccagttag gactgttatg aatactattc aacaattaat |
| 1321 | ggtgatttta aattctgcaa gtgatcagcc atcagaaaat ctgatttcct acttcaataa |
| 1381 | ttgcacagtg aatccaaaag aaaatatcct aaagagagta aaggatgttg ggcacatctt |
| 1441 | taaagagaag tttgctaacg ctgtgggcca gggctgtgtt gacatcggag tacagcgata |
| 1501 | taaacttgga gtccgattgt attaccgtgt gatggaatcc atgcttaaat cagaagaaga |
| 1561 | acgtttgtcc attcagaatt ttagcaaact cctaaatgac aacatctttc atatgtcttt |
| 1621 | actggcctgt gctcttgaag ttgtaatggc tacgtatagc agaagtacat tgcagcatct |
| 1681 | tgattctgga acagatttgt ccttcccgtg gattctgaac gtacttaatt taaaagcctt |
| 1741 | tgatttttac aaagtgattg aaagttttat caaagtggaa gccaacttga caagagaaat |
| 1801 | gataaaacat ttagaaagat gtgagcatcg aatcatggaa tcccttgcat ggctttcaga |
| 1861 | ttcaccttta tttgatctca ttaagcagtc caaggatgga gaaggacctg ataaccttga |
| 1921 | acctgcttgt cctctcagcc tgcctctcca gggtaaccat actgcagcag atatgtatct |
| 1981 | ttctcctcta agatctccaa agaaaagaac ttccactaca cgtgtaaatt ctgctgcaaa |
| 2041 | tacagagaca caagcagcct cagccttcca tactcagaag ccattgaaat ctacctccct |
| 2101 | tgccctgttt tacaaaaaag tgtaccgtct agcatatctc cgactaaata cactctgtgc |
| 2161 | acgccttctg tctgaccacc cagagctaga gcacatcatc tggactctgt ttcagcatac |
| 2221 | attgcaaaat gagtatgagc tcatgagaga ccgacatttg gaccagatta tgatgtgctc |
| 2281 | tatgtatggc atctgcaagg tgaagaacat cgacctcaag ttcaaaatca tcgtcactgc |
| 2341 | ctacaaggat cttcctcacg ctgcccagga gacctttaaa cgtgttttga tcagagaaga |
| 2401 | ggagtttgat tccattatag tattctataa ctccgttttc atgcagagac taaaaacaaa |
| 2461 | tattttacag tatgcctcca ccaggcctcc taccttgtca ccaatacctc acattcctcg |
| 2521 | aagcccttac aagttttcta gttcaccctt acggattcct ggaggtaaca tctatatatc |
| 2581 | acccctaaag agtccttata aaatttcaga aggtctgcca acacccacaa aaatgactcc |
| 2641 | gagatcaaga atcttggtct caattggtga atcatttggg acatctgaaa agttccagaa |
| 2701 | aataaaccag atggtgtgta atagtgacag agtgctcaaa agaagtgctg aaggcggcaa |
| 2761 | cccccccaaa ccactgaaaa agctgcgctt tgacatcgag ggagccgatg aagcagatgg |
| 2821 | gagtaaacat ctcccagcgg agtccaaatt ccaacagaaa ctggcagaaa tgacttccac |
| 2881 | tcgaacacga atgcaaaagc agagaatgaa tgagagcaag gatgtctcaa acaaggagga |
| 2941 | aaagtgagga cctcagggcc ctggaccctc agccctgggg acaccagact cctggctcat |
| 3001 | ggttgtgact agttcccagg ttctgctcat gttagagata taaaatgtgc aggtacaagc |
| 3061 | tgaatatttg tgtgggtgat tcgtaagcca cttcaatgtt gtaatcaatg gtagagaatt |
| 3121 | gatagcacac aggattgaga atcttgtgca gatcatgcca tttaaaatat gaaagcaagt |
| 3181 | tgtttgcatt tccaacatga gcctgctgcc ctcagaagtg agggcctctg caggggtcct |
| 3241 | gaccccctac atctggctga ctactttgcc tttcctcatg gcacatgtgt gatgtttgct |
| 3301 | cttggtttta ttaatttata tgtatttttt aatttaacgt gaataccctt agaaaatgtg |
| 3361 | tcttgtcttc caaatgcaag ttgattgact gtccacattc aaccaaatta tcttgaatct |
| 3421 | tctactgaaa cagattgtta caaactggga aaaagtacta atttctacac attggactat |
| 3481 | tttaatatta gaatctaatg tgccgtgtgt ttgtttcaca tattttactt ttgaaataca |
| 3541 | agcaaaaaaa gtataatcat atgatactgt cttactactg acacagattt catacctcag |
| 3601 | accctctaag aaccgattct tttattcacc caacacatgc tttgaactga agactattga |
| 3661 | taatactccg aggttgtttt tttctttcaa tcattcagat cactgaattt ataagtaccc |
| 3721 | atgtagtact tgaaagtcaa gtttggccac aactgtgctt aagaggaccc tagtacagta |
| 3781 | caacccaagt gcacttttta atgtttctgg gtcctgaaga atcaagatac aaattaattg |
| 3841 | tgatttacaa gcagactgtt aactatagaa gccttcagtt tttttccctc atagacgtgt |
| 3901 | ctaattacat ctcaacagtt tactctgttc ttctacatct ggggatgttt gtgttctctc |
| 3961 | tggaatggta catcttccag ggtcttttga acttgcagtt atctattttt taagccaatc |
| 4021 | tggtctaata actctggctt cttcaaagcc acaccatttc tagtccagct gtgcagaaac |
| 4081 | ttcagatgaa aacagctgca ttgaaaatag aggcactccc ttcacccccc acctaaaggt |
| 4141 | gtatttaaat tatcttgtgg gattaactta tttagagatg gtataattta aaatagggga |
| 4201 | tatttaaggt agcatcagct agcatttaag aaaatcactt tttctaaact ccatactttt |
| 4261 | tgaaaagaaa tctggtcttg ttaggaaaca aatttctatt ttgtcctcaa tttagtttca |
| 4321 | gttttactag tttgatagtt atctcaataa caaaagcaat agacagcttc ccccatttct |
| 4381 | tcattaagtt ttgcatgatc atcacacaga ttagttaggt tttaggtcaa gggcttacca |
| 4441 | tacttctagg tcttttgcta gtgagttcaa gttagaatta gtgacagaat cataggaatt |
| 4501 | ttcagagatc ctgcttcgag atttcttaaa gctgcagaca ctgcactatt ggttttgttt |
| 4561 | ttttgtaccg gttgaaacta tacattcaaa ttgctatgtt cctattttct ataatagttt |
| 4621 | gtctatttta aaataaacta gttgttcaga gccttaaaaa aaaaaaaaaa aaa |

SEQ ID NO: 36 Human CYLD Isoform 1 Amino Acid Sequence (NP_056062.1)

| | |
|---|---|
| 1 | mssglwsqek vtspyweeri fylllqecsv tdkqtqkllk vpkgsigqyi qdrsvghsri |
| 61 | psakgkknqi qlkileqpha vlfvdekdvv einekftell laitnceerf slfknrnrls |
| 121 | kglqidvgcp vkvqlrsgee kfpgvvrfrg pllaertvsg iffgvellee grgqgftdgv |
| 181 | yqgkqlfqcd edcgvfvald kleliedddt alesdyagpg dtmqvelppl einsrvslkv |
| 241 | getiesgtvi fcdvlpgkes lgyfvgvdmd npignwdgrf dgvqlcsfac vestillhin |
| 301 | diipalsesv tqerrppkla fmsrgvgdkg ssshnkpkat gstsdpgnrn rselfytlng |
| 361 | ssvdsqpqsk skntwyidev aedpakslte istdfdrssp plqpppvnsl ttenrfhslp |
| 421 | fsltkmpntn gsighsplsl saqsvmeeln tapvqesppl amppgnshgl evgslaevke |
| 481 | nppfygvirw igqppglnev lagleledec agctdgtfrg tryftcalkk alfvklkscr |
| 541 | pdsrfaslqp vsnqiercns lafggylsev veentppkme kegleimigk kkgiqghyns |
| 601 | cyldstlfcl fafssvldtv llrpkekndv eyysetqell rteivnplri ygyvcatkim |
| 661 | klrkilekve aasgftseek dpeeflnilf hhilrvepll kirsagqkvq dcyfyqifme |
| 721 | knekvgvpti qqllewsfin snlkfaeaps cliiqmprfg kdfklfkkif pslelnitdl |
| 781 | ledtprqcri cgglamyecr ecyddpdisa gkikqfcktc ntqvhlhpkr lnhkynpvsl |
| 841 | pkdlpdwdwr hgcipcqnme lfavlciets hyvafvkygk ddsawlffds madrdggqng |
| 901 | fnipqvtpcp evgeylkmsl edlhsldsrr iqgcarrllc daymcmyqsp tmslyk |

SEQ ID NO: 37 Human CYLD transcript variant 1 cDNA sequence (NM_015247.2;
CDS: 416-3286)

| | |
|---|---|
| 1 | gtgcggttcg gaggcggggc aggtgggggc gggcccaggt agcaggtttg gctgcgcggg |
| 61 | ggccgcgcgt cggagtttcc ccctttctag ggtgaggatg gttctacaca gccacccgga |
| 121 | gttccttagt tgaaaggtgc gccctgctgt gacagaatgt ggtaattgta atctttaaca |
| 181 | ttttcatgta aaacatattt cctgatcatc tttccattgt cttcatggaa aattgataaa |
| 241 | tatttgtgcc ttccaactct cgtcttggtt gaatgacttc atcttaatac aacatggaca |
| 301 | ccacgttgct gaaaacatgc tttgggactg ccactgaatt tatcttttgc ggttttatga |
| 361 | caaagttatt agtagtttcc cttttttgaa ttagtatttt gaagttaata tcacaatgag |
| 421 | ttcaggctta tggagccaag aaaaagtcac ttcaccctac tgggaagagc ggatttttta |
| 481 | cttgcttctt caagaatgca gcgttacaga caaacaaaca caaaagctcc ttaaagtacc |
| 541 | gaagggaagt ataggacagt atattcaaga tcgttctgtg gggcattcaa ggattccttc |
| 601 | tgcaaaaggc aagaaaaatc agattggatt aaaaattcta gagcaacctc atgcagttct |
| 661 | ctttgttgat gaaaaggatg ttgtagagat aaatgaaaag ttcacagagt tacttttggc |
| 721 | aattaccaat tgtgaggaga ggttcagcct gtttaaaaac agaaacagac taagtaaagg |
| 781 | cctccaaata gacgtgggct gtcctgtgaa agtacagctg agatctgggg aagaaaaatt |
| 841 | tcctggagtt gtacgcttca gaggacccct gttagcagag aggacagtct ccggaatatt |
| 901 | ctttggagtt gaattgctgg aagaaggtcg tggtcaaggt ttcactgacg gggtgtacca |
| 961 | agggaaacag ctttttcagt gtgatgaaga ttgtggcgtg tttgttgcat tggacaagct |
| 1021 | agaactcata gaagatgatg acactgcatt ggaaagtgat tacgcaggtc ctggggacac |
| 1081 | aatgcaggtc gaacttcctc ctttggaaat aaactccaga gtttctttga aggttggaga |
| 1141 | aacaatagaa tctggaacag ttatattctg tgatgttttg ccaggaaaag aaagcttagg |
| 1201 | atattttgtt ggtgtggaca tggataaccc tattggcaac tgggatggaa gatttgatgg |
| 1261 | agtgcagctt tgtagttttg cgtgtgttga aagtacaatt ctattgcaca tcaatgatat |
| 1321 | catcccagct ttatcagaga gtgtgacgca ggaaaggagg cctcccaaac ttgcctttat |
| 1381 | gtcaagaggt gttggggaca aaggttcatc cagtcataat aaaccaaagg ctacaggatc |
| 1441 | tacctcagac cctggaaata gaaacagatc tgaattattt tataccttaa atgggtcttc |
| 1501 | tgttgactca caaccacaat ccaaatcaaa aaatacatgg tacattgatg aagttgcaga |
| 1561 | agaccctgca aaatctctta cagagatatc tacagacttt gaccgttctt caccaccact |
| 1621 | ccagcctcct cctgtgaact cactgaccac cgagaacaga ttccactctt taccattcag |
| 1681 | tctcaccaag atgcccaata ccaatggaag tattggccac agtccacttt ctctgtcagc |
| 1741 | ccagtctgta atggaagagc taaacactgc acccgtccaa gagagtccac ccttggccat |
| 1801 | gcctcctggg aactcacatg gtctagaagt gggctcattg gctgaagtta aggagaaccc |
| 1861 | tcctttctat ggggtaatcc gttggatcgg tcagccacca ggactgaatg aagtgctcgc |
| 1921 | tggactggaa ctggaagatg agtgtgcagg ctgtacggat ggaaccttca gaggcactcg |
| 1981 | gtatttcacc tgtgccctga agaaggcgct gtttgtgaaa ctgaagagct gcaggcctga |
| 2041 | ctctaggttt gcatcattgc agccggtttc caatcagatt gagcgctgta actctttagc |
| 2101 | atttggaggc tacttaagtg aagtagtaga agaaaatact ccaccaaaaa tggaaaaaga |
| 2161 | aggcttggag ataatgattg ggaagaagaa aggcatccag ggtcattaca attcttgtta |
| 2221 | cttagactca accttattct gcttatttgc ttttagttct gttctggaca ctgtgttact |
| 2281 | tagacccaaa gaaaagaacg atgtagaata ttatagtgaa acccaagagc tactgaggac |
| 2341 | agaaattgtt aatcctctga gaatatatgg atatgtgtgt gccacaaaaa ttatgaaact |
| 2401 | gaggaaaata cttgaaaagg tggaggctgc atcaggattt acctctgaag aaaaagatcc |
| 2461 | tgaggaattc ttgaatattc tgtttcatca tattttaagg gtagaacctt tgctaaaaat |
| 2521 | aagatcagca ggtcaaaagg tacaagattg ttacttctat caaattttta tggaaaaaaa |
| 2581 | tgagaaagtt ggcgttccca caattcagca gttgttagaa tggtctttta tcaacagtaa |
| 2641 | cctgaaattt gcagaggcac catcatgtct gattattcag atgcctcgat ttggaaaaga |
| 2701 | ctttaaacta tttaaaaaaa tttttccttc tctggaatta aatataacag atttacttga |
| 2761 | agacactccc agacagtgcc ggatatgtgg agggcttgca atgtatgagt gtagagaatg |
| 2821 | ctacgacgat ccggacatct cagctggaaa aatcaagcag ttttgtaaaa cctgcaacac |
| 2881 | tcaagtccac cttcatccga agaggctgaa tcataaatat aacccagtgt cacttcccaa |
| 2941 | agacttaccc gactgggact ggagacacgg ctgcatccct tgccagaata tggagttatt |
| 3001 | tgctgttctc tgcatagaaa caagccacta tgttgctttt gtgaagtatg ggaaggacga |
| 3061 | ttctgcctgg ctcttctttg acagcatggc cgatcgggat ggtggtcaga atggcttcaa |
| 3121 | cattcctcaa gtcaccccat gcccagaagt aggagagtac ttgaagatgt ctctggaaga |
| 3181 | cctgcattcc ttggactcca ggagaatcca aggctgtgca cgaagactgc tttgtgatgc |
| 3241 | atatatgtgc atgtaccaga gtccaacaat gagtttgtac aaataactgg ggtcatcggg |
| 3301 | aaaggcaaag aaactgaagg cagagtccta acgttgcatc ttattcgagc tggcagttct |
| 3361 | gttcacgtcc attgccggca atggatgtct ttgtggtgat gatccttcag aaaaggatgc |
| 3421 | ctctgtttaa aaacaaattg cttttgtgtc cctgaagtat ttaataagaa gcattttgca |
| 3481 | ctctagaaag tatgtttgtg ttggtttttt aagaagtcta aatgaagtta ttaatacctg |
| 3541 | aagctttaag ttaagtgcat tgatcatatg atatttttgg aagcatacaa ttttaattgt |
| 3601 | ggaagtttaa agcctctttt agtccattga gaatgtaaat aaatgtgtct tctttatgga |
| 3661 | ccaaggatat gaaatcattt ttcttttgta gctaacggtt gccttgagga agaaataatt |
| 3721 | tggttttatt aagagtctac tctcaatcca gttattagag atgtactgag tttgatttgt |
| 3781 | taatcctttc tatatactgc tgatcttgca tgtctacaat ctgctcagtt tttctgtgtt |
| 3841 | tctgcaatag tggtcagaaa aatacttaaa ttcccttaat ggtgttgttt tctatttgtt |
| 3901 | ctggttttga gataaatgag tgattctgtc cccaaatgtc catttttgaa gtgattttcc |
| 3961 | tggaggatta gggtatttag cagttgaagc tcttcattca tagtagttac tgtcagctaa |
| 4021 | caggtttttt aaggctttta actattaata ttttatggaa tggggcaaag taaattgatg |
| 4081 | aaagaattgg agtgataata gtcctttaca aacatacagt ccataagaaa atgaatttgg |
| 4141 | catatagaat tattacaatt tcctgggaga gatggatatt taaacctcta ttattttaga |
| 4201 | caagactgtc tagaacttaa gtttgatctg tcagccagta ctcccattaa attcagtgta |
| 4261 | gtttcacttg atagaatcag atatgttatc gaaatgttag cagcagcttc atcctccttc |
| 4321 | tgattaaagt aagtagaaat gggatgtttt gtttaataac agccatagtg tgtgtttaga |
| 4381 | ccacagcgga tgttgtagac caggaccata gatgatacat gtcagtgctg tggaatgtgc |
| 4441 | attctctgag tgttgttttg tggtatcatt gtctttcctg aatgactttc taactgtgca |
| 4501 | gaaaggcaga aaagtcatca tatgtatatg tcatatgact ttataaaata tttaatgtga |
| 4561 | caaaaagtgg aaagaatctt tacaaaccct gcaattactt ttttaaaggc acttttactc |
| 4621 | tttggtttta tcattccatt ttgctaatat ttactagctt tataaattac agtaaggtac |
| 4681 | aaaaactcat cttgtaatat tttcattttt gaagtgaaaa agtacatata ttttgcacaa |
| 4741 | ggttttatac tgctaagtgc ttggttgggg tggtgagatg atgattagat caggggtgag |
| 4801 | gctgagagac tctgggttta gggctagccc tgcctccatc tcccttgggt aaaatgaagg |
| 4861 | gtgtggggta aaagatgcat aaggcctttt ctagctctga cagcctagaa gtccaatcac |
| 4921 | cctgtaataa atatgtgttg aatgaagaaa tgggtgaatg agcttgtcaa tgtgatttta |
| 4981 | aaaaattgac tacctggagg aatgattagg aatctaaatg aagccagccc tcggtatctg |
| 5041 | caggtttctc atccatggat tcaaccaact gcaaatggaa aatacgattt tttttaaaaa |
| 5101 | aaggatggtt acatccgtat tgaacatgta cagacttttt tcttgtcatt attctctgaa |
| 5161 | caatacaaga actctttatg tagcatttac atttattagg tattataagt aatctagaga |
| 5221 | ttatttaatt aaaatataca ggaggatgtg tgtcagttat atgcaaattc tgtaccattt |
| 5281 | tgtatcaggg aattgagcat cttcagatgt tggtatctgc agggatcctg gaaccaaacc |
| 5341 | cctgcagata ctaagggctg acgatctagg taagactgga tttaacagtt ggaaaaaaaa |
| 5401 | aaaaaaaagg agagagaaga cagttccttt cctgtagaaa ttaaaacaaa atacaaattg |
| 5461 | aggaagctct gctacccagg ctgtcatggt agagaacttg aagaagacct gtttggatgg |
| 5521 | acacctggtt tcaaaagtca ggtgtggaga ctgttaaatg ggagggcctc atccataaat |
| 5581 | gatttctggc aacgtcttct tcaggtggag cttgacgtct ttttaatgtt acttggggag |
| 5641 | ggagtgctca ttaagggatg ccagggccag ctctggtggt tcctggggag gctgcgtcct |
| 5701 | tccctgcttc tgcatgtcat gaggcagcag gaaggtttcc cctgcacctg tctgtcctgg |
| 5761 | ctccctctgg gtagccccct actgttctgt gcttcagcac agcctggttt gtcaagaggc |
| 5821 | acatagttgg ggctgggctg catggcacag gggcttatgt gcctgctggt tatttaattt |
| 5881 | tcagccttaa gttttcttta atattttcct gttggctatt taaaggtttt ggttatcttt |
| 5941 | tattccttat ctacaatcaa gatgacaatg taattgaatt atcttattta taacacggtt |
| 6001 | cgtgattcat gattcatgat tacaagtaga aaatatgtca tgttcctcac ctccaaataa |
| 6061 | atatgtgtgt gtctgtgtgt gtgtatatat gtatgtggcg gagagggaga gagtggggaa |
| 6121 | ggagagcagt gttatcatac atagagaggc taaatgtgtc ccatccctca ctgtcagctt |
| 6181 | tataaaggag tttgactcca tccacagaag aatgttttat aagactagga aaacacgttg |
| 6241 | aaaactagga taaacagcaa caaaaatcaa ctaaatatgt tgttactgtt gctaaggatt |
| 6301 | ttctccttag aataatttag gatttttaaa aatttctgtt tgccaaatgc tgtagataaa |
| 6361 | tggccagatt cttcctatcc ctaggattcc tttattattt tttttcacag attttgagaa |
| 6421 | caagggggag agatagtatg gaagattaag attccattaa tcttatagaa ctgtgttgtc |
| 6481 | acccaaattc ctgcttgttt gaacatggca tcttcataga ttcaggattc actaccctct |
| 6541 | atagctggat cttgaaaatt atctggccag ataattttgc atctgcttgg atgattgtag |
| 6601 | actgagatgt gagtggagga taaagtatta gacttttgct gagtaactgc caaccaagaa |
| 6661 | gtatttatcg gacacttact aggtgcctag gattgtatca gagggaatat gaaatgtgtc |
| 6721 | cctgccctac ctagttttaa cgacagaata tctattaaag gctacttagc tgaagggtaa |
| 6781 | gggtgacagg tctaggggaa gctttgggag gtggtgtgct gtgacagaaa aagtggcaga |
| 6841 | gtagggacga gagacctgca ttctagccct gtttctgtca cttgctctgt acacttagac |
| 6901 | aacagcttga cctcttgagc tttagtttcc tcctctgcat aatgagaggg ttagactact |
| 6961 | gaattgtatg ggaaaaaaat acaaattcct gggtcctagg ccatgcctgc tgaatccgac |
| 7021 | tgttcaggaa gaggcctagg aaatctgtga gggaatcccc aggggaatct cgtgaccagc |
| 7081 | caggtgtgaa atctgctaac tggaagatct caaagcttcc ttcacttttt gtgattttgt |
| 7141 | ggtcatgtaa cgttactgta ttattctacg taaatgtggg tacttggatg tttatcatac |
| 7201 | tgtttctctg tgtttacata ctaatttgtg taagaaatgc attttagtct gtgtacctca |
| 7261 | acctgctgtt tgtttcctag aggtgttagt agtctttaaa tacaagtaag acttaagagg |
| 7321 | atatttgatg ttatttacct ggatattttc ttcccctttt atttatttag aggaaattga |
| 7381 | gattctagga gccaaaaaaa tgaaaacaaa attctaaggc aaagttaaag aaaaaaatta |
| 7441 | cattatttct taccatttgc tactttataa tgaaaattta aaaattatat gggaagattt |
| 7501 | ttctctggga taacaaatcc ttgtcataaa gtaagaggtc tttttaaagt aggtaggcta |
| 7561 | taaggcctgt aatttaaaat aatactcctt tctctagggt ttggtgcaat tctccattaa |
| 7621 | tgaagataac atttgaattc cccaaagcag gtgaggagtc ggggaggaga aagcgatgtt |
| 7681 | aaaatgaaaa ctcactgcaa aagaggaggc agaggaagaa ggaatgtaaa ccccttaaag |
| 7741 | cagatgtgtg tggggcctta tgaagaccag gattctgcgg gtgtcagggg attgcccctc |
| 7801 | ttgacagaga ctagggtttt agactgaggc ttcctgcagg gtgttcgcat tgcccttctc |
| 7861 | cgttcccctt cagacctttc tggggagaag aggtgggagg aggggagaaa gactgttcat |
| 7921 | cttattctga atcctggagc agctgaaggt tttctcttga gtcaggatgc agtggtaatg |
| 7981 | cattaaccag caagtgtggc caaggataat gaaaaagtgg gaaaggaagg tcctcctcct |
| 8041 | ccctgattgt agcatccagc agtctctgta gccaggttac tcaagaacca catttgattt |
| 8101 | cctggccctt tgccttggca gtgatggcat ttttatttca ctgtgtttta aagtcttcat |
| 8161 | ttatttttat aacatgggtt agggagaagg gccacaaatg gagggattgt cctttcaagc |
| 8221 | accacagctt cagataaaat tagtactttc aaatattgtc cactttaact taaaaaattc |
| 8281 | tagagggatt atattggaga ctcaactgcc cttggtttta gtttataaaa tggcctagta |
| 8341 | ctgtggaatt ttaattttag aaagtcttag catcagatca taaacattca ttaaaagaac |
| 8401 | tcacatccca tctgaaactt cccaggggag ttgggattct tagtagattg gtagaaaggg |
| 8461 | gctcattttc tactgcattt cccatttttg gtatcttgtt cagcatgttt tatttttatt |
| 8521 | tcttgtctgc agaacatcct atatttatga gaacattctt taagaagacc accacataga |
| 8581 | ataccccttc ctatcagctc gctctgattt agccttaatt ttgttaaatt ttttagagat |
| 8641 | gaatgaagtg ctgctgtgga aagaaatgta catatactat ttctgtatca ttaaaattac |
| 8701 | atttttatgg ttcaaaaaaa aaaaaaaaaa |

SEQ ID NO: 38 Human CYLD Isoform 2 Amino Acid Sequence (NP_001035814.1
and NP_001035877.1)

| | |
|---|---|
| 1 | mssglwsqek vtspyweeri fylllqecsv tdkqtqkllk vpkgsigqyi qdrsvghsri |
| 61 | psakgkknqi glkileqpha vlfvdekdvv einekftell laitnceerf slfknrnrls |
| 121 | kglqidvgcp vkvqlrsgee kfpgvvrfrg pllaertvsg iffgvellee grgqgftdgv |
| 181 | yqgkqlfqcd edcgvfvald kleliedddt alesdyagpg dtmqvelppl einsrvslkv |
| 241 | getiesgtvi fcdvlpgkes lgyfvgvdmd npignwdgrf dgvqlcsfac vestillhin |
| 301 | diipesvtqe rrppklafms rgvgdkgsss hnkpkatgst sdpgnrnrse lfytlngssv |
| 361 | dsqpqskskn twyidevaed pakslteist drdrsspplq pppvnsltte nrfhslpfsl |
| 421 | tkmpntngsi ghsplslsaq svmeelntap vqespplamp pgnshglevg slaevkenpp |
| 481 | fygvirwigq ppglnevlag leledecagc tdgtfrgtry ftcalkkalf vklkscrpds |
| 541 | rfaslqpvsn qiercnslaf ggylsevvee ntppkmekeg leimigkkkg iqghynscyl |
| 601 | dstlfclfaf ssvldtvllr pkekndveyy setqellrte ivnplriygy vcatkimklr |
| 661 | kilekveaas gftseekdpe eflnilfhhi lrvepllkir sagqkvqdcy fyqifmekne |
| 721 | kvgvptiqql lewsfinsnl kfaeapscli iqmprfgkdf klfkkifpsl elnitdlled |
| 781 | tprqcricgg lamyecrecy ddpdisagki kqfcktcntq vhlhpkrlnh kynpvslpkd |
| 841 | lpdwdwrhgc ipcqnmelfa vlcietshyv afvkygkdds awlffdsmad rdggqngfni |
| 901 | pqvtpcpevg eylkmsledl hsldsrriqg carrllcday mcmyqsptms lyk |

SEQ ID NO: 39 Human CYLD transcript variant 2 cDNA sequence
(NM_001042355,2; CDS: 243 . . . 3104)

| | |
|---|---|
| 1 | aggtagcagg tttggctgcg cgggggccgc gcgtcggagt ttcccccttt ctagggtgag |
| 61 | gatggttcta cacagccacc cggagttcct tagttgaaag gtgcgccctg ctgtgacagc |
| 121 | atggacacca cgttgctgaa aacatgcttt gggactgcca ctgaatttat cttttgcggt |
| 181 | tttatgacaa agttattagt agtttccctt ttttgaatta gtattttgaa gttaatatca |
| 241 | caatgagttc aggcttatgg agccaagaaa aagtcacttc accctactgg gaagagcgga |
| 301 | ttttttactt gcttcttcaa gaatgcagcg ttacagacaa acaaacacaa aagctcctta |
| 361 | aagtaccgaa gggaagtata ggacagtata ttcaagatcg ttctgtgggg cattcaagga |
| 421 | ttccttctgc aaaaggcaag aaaaatcaga ttggattaaa aattctagag caacctcatg |
| 481 | cagttctctt tgttgatgaa aaggatgttg tagagataaa tgaaaagttc acagagttac |
| 541 | ttttggcaat taccaattgt gaggagaggt tcagcctgtt taaaaacaga aacagactaa |
| 601 | gtaaaggcct ccaaatagac gtgggctgtc ctgtgaaagt acagctgaga tctggggaag |
| 661 | aaaaatttcc tggagttgta cgcttcagag gacccctgtt agcagagagg acagtctccg |
| 721 | gaatattctt tggagttgaa ttgctggaag aaggtcgtgg tcaaggtttc actgacgggg |
| 781 | tgtaccaagg gaaacagctt tttcagtgtg atgaagattg tggcgtgttt gttgcattgg |
| 841 | acaagctaga actcatagaa gatgatgaca ctgcattgga aagtgattac gcaggtcctg |
| 901 | gggacacaat gcaggtcgaa cttcctcctt tggaaataaa ctccagagtt tctttgaagg |
| 961 | ttggagaaac aatagaatct ggaacagtta tattctgtga tgttttgcca ggaaaagaaa |
| 1021 | gcttaggata ttttgttggt gtggacatgg ataaccctat tggcaactgg gatggaagat |
| 1081 | ttgatggagt gcagctttgt agttttgcgt gtgttgaaag tacaattcta ttgcacatca |
| 1141 | atgatatcat cccagagagt gtgacgcagg aaaggaggcc tcccaaactt gcctttatgt |
| 1201 | caagaggtgt tggggacaaa ggttcatcca gtcataataa accaaaggct acaggatcta |
| 1261 | cctcagaccc tggaaataga aacagatctg aattatttta taccttaaat gggtcttctg |
| 1321 | ttgactcaca accacaatcc aaatcaaaaa atacatggta cattgatgaa gttgcagaag |
| 1381 | accctgcaaa atctcttaca gagatatcta cagactttga ccgttcttca ccaccactcc |
| 1441 | agcctcctcc tgtgaactca ctgaccaccg agaacagatt ccactcttta ccattcagtc |
| 1501 | tcaccaagat gcccaatacc aatggaagta ttggccacag tccactttct ctgtcagccc |
| 1561 | agtctgtaat ggaagagcta aacactgcac ccgtccaaga gagtccaccc ttggccatgc |
| 1621 | ctcctgggaa ctcacatggt ctagaagtgg gctcattggc tgaagttaag gagaaccctc |
| 1681 | ctttctatgg ggtaatccgt tggatcggtc agccaccagg actgaatgaa gtgctcgctg |
| 1741 | gactggaact ggaagatgag tgtgcaggct gtacggatgg aaccttcaga ggcactcggt |
| 1801 | atttcacctg tgccctgaag aaggcgctgt ttgtgaaact gaagagctgc aggcctgact |
| 1861 | ctaggtttgc atcattgcag ccggtttcca atcagattga gcgctgtaac tctttagcat |
| 1921 | ttggaggcta cttaagtgaa gtagtagaag aaaatactcc accaaaaatg gaaaaagaag |
| 1981 | gcttggagat aatgattggg aagaagaaag gcatccaggg tcattacaat tcttgttact |
| 2041 | tagactcaac cttattctgc ttatttgctt ttagttctgt tctggacact gtgttactta |
| 2101 | gacccaaaga aaagaacgat gtagaatatt atagtgaaac ccaagagcta ctgaggacag |
| 2161 | aaattgttaa tcctctgaga atatatggat atgtgtgtgc cacaaaaatt atgaaactga |
| 2221 | ggaaaatact tgaaaaggtg gaggctgcat caggatttac ctctgaagaa aaagatcctg |
| 2281 | aggaattctt gaatattctg tttcatcata ttttaagggt agaacctttg ctaaaaataa |
| 2341 | gatcagcagg tcaaaaggta caagattgtt acttctatca aatttttatg gaaaaaaatg |
| 2401 | agaaagttgg cgttcccaca attcagcagt tgttagaatg gtcttttatc aacagtaacc |
| 2461 | tgaaatttgc agaggcacca tcatgtctga ttattcagat gcctcgattt ggaaaagact |
| 2521 | ttaaactatt taaaaaaatt tttccttctc tggaattaaa tataacagat ttacttgaag |
| 2581 | acactcccag acagtgccgg atatgtggag ggcttgcaat gtatgagtgt agagaatgct |
| 2641 | acgacgatcc ggacatctca gctggaaaaa tcaagcagtt ttgtaaaacc tgcaacactc |
| 2701 | aagtccacct tcatccgaag aggctgaatc ataaatataa cccagtgtca cttcccaaag |
| 2761 | acttacccga ctgggactgg agacacggct gcatcccttg ccagaatatg gagttatttg |
| 2821 | ctgttctctg catagaaaca agccactatg ttgcttttgt gaagtatggg aaggacgatt |
| 2881 | ctgcctggct cttctttgac agcatggccg atcgggatgg tggtcagaat ggcttcaaca |
| 2941 | ttcctcaagt caccccatgc ccagaagtag gagagtactt gaagatgtct ctggaagacc |
| 3001 | tgcattcctt ggactccagg agaatccaag gctgtgcacg aagactgctt tgtgatgcat |
| 3061 | atatgtgcat gtaccagagt ccaacaatga gtttgtacaa ataactgggg tcatcgggaa |
| 3121 | aggcaaagaa actgaaggca gagtcctaac gttgcatctt attcgagctg gcagttctgt |
| 3181 | tcacgtccat tgccggcaat ggatgtcttt gtggtgatga tccttcagaa aaggatgcct |
| 3241 | ctgtttaaaa acaaattgct tttgtgtccc tgaagtattt aataagaagc attttgcact |
| 3301 | ctagaaagta tgtttgtgtt ggttttttaa gaagtctaaa tgaagttatt aatacctgaa |
| 3361 | gctttaagtt aagtgcattg atcatatgat atttttggaa gcatacaatt ttaattgtgg |
| 3421 | aagtttaaag cctcttttag tccattgaga atgtaaataa atgtgtcttc tttatggacc |
| 3481 | aaggatatga aatcattttt cttttgtagc taacggttgc cttgaggaag aaataatttg |
| 3541 | gttttattaa gagtctactc tcaatccagt tattagagat gtactgagtt tgatttgtta |
| 3601 | atcctttcta tatactgctg atcttgcatg tctacaatct gctcagtttt tctgtgtttc |
| 3661 | tgcaatagtg gtcagaaaaa tacttaaatt cccttaatgg tgttgttttc tatttgttct |
| 3721 | ggttttgaga taaatgagtg attctgtccc caaatgtcca tttttgaagt gattttcctg |
| 3781 | gaggattagg gtatttagca gttgaagctc ttcattcata gtagttactg tcagctaaca |
| 3841 | ggttttttaa ggcttttaac tattaatatt ttatggaatg gggcaaagta aattgatgaa |
| 3901 | agaattggag tgataatagt cctttacaaa catacagtcc ataagaaaat gaatttggca |
| 3961 | tatagaatta ttacaatttc ctgggagaga tggatattta aacctctatt attttagaca |
| 4021 | agactgtcta gaacttaagt ttgatctgtc agccagtact cccattaaat tcagtgtagt |
| 4081 | ttcacttgat agaatcagat atgttatcga aatgttagca gcagcttcat cctccttctg |
| 4141 | attaaagtaa gtagaaatgg gatgttttgt ttaataacag ccatagtgtg tgtttagacc |
| 4201 | acagcggatg ttgtagacca ggaccataga tgatacatgt cagtgctgtg gaatgtgcat |
| 4261 | tctctgagtg ttgttttgtg gtatcattgt ctttcctgaa tgactttcta actgtgcaga |
| 4321 | aaggcagaaa agtcatcata tgtatatgtc atatgacttt ataaaatatt taatgtgaca |
| 4381 | aaaagtggaa agaatcttta caaaccctgc aattactttt ttaaaggcac ttttactctt |
| 4441 | tggttttatc attccatttt gctaatattt actagcttta taaattacag taaggtacaa |
| 4501 | aaactcatct tgtaatattt tcatttttga agtgaaaaag tacatatatt ttgcacaagg |
| 4561 | ttttatactg ctaagtgctt ggttggggtg gtgagatgat gattagatca ggggtgaggc |
| 4621 | tgagagactc tgggtttagg gctagccctg cctccatctc ccttgggtaa aatgaagggt |
| 4681 | gtggggtaaa agatgcataa ggccttttct agctctgaca gcctagaagt ccaatcaccc |
| 4741 | tgtaataaat atgtgttgaa tgaagaaatg ggtgaatgag cttgtcaatg tgattttaaa |
| 4801 | aaattgacta cctggaggaa tgattaggaa tctaaatgaa gccagccctc ggtatctgca |
| 4861 | ggtttctcat ccatggattc aaccaactgc aaatggaaaa tacgattttt tttaaaaaaa |
| 4921 | ggatggttac atccgtattg aacatgtaca gacttttttc ttgtcattat tctctgaaca |
| 4981 | atacaagaac tctttatgta gcatttacat ttattaggta ttataagtaa tctagagatt |
| 5041 | atttaattaa aatatacagg aggatgtgtg tcagttatat gcaaattctg taccattttg |
| 5101 | tatcagggaa ttgagcatct tcagatgttg gtatctgcag ggatcctgga accaaacccc |
| 5161 | tgcagatact aagggctgac gatctaggta agactggatt taacagttgg aaaaaaaaaa |
| 5221 | aaaaaaggag agagaagaca gttcctttcc tgtagaaatt aaaacaaaat acaaattgag |
| 5281 | gaagctctgc tacccaggct gtcatggtag agaacttgaa gaagacctgt ttggatggac |
| 5341 | acctggtttc aaaagtcagg tgtggagact gttaaatggg agggcctcat ccataaatga |
| 5401 | tttctggcaa cgtcttcttc aggtggagct tgacgtcttt ttaatgttac ttggggaggg |
| 5461 | agtgctcatt aagggatgcc agggccagct ctggtggttc ctggggaggc tgcgtccttc |
| 5521 | cctgcttctg catgtcatga ggcagcagga aggtttcccc tgcacctgtc tgtcctggct |
| 5581 | ccctctgggt agccccctac tgttctgtgc ttcagcacag cctggtttgt caagaggcac |
| 5641 | atagttgggg ctgggctgca tggcacaggg gcttatgtgc ctgctggtta tttaattttc |
| 5701 | agccttaagt tttctttaat attttcctgt tggctattta aaggttttgg ttatctttta |
| 5761 | ttccttatct acaatcaaga tgacaatgta attgaattat cttatttata acacggttcg |
| 5821 | tgattcatga ttcatgatta caagtagaaa atatgtcatg ttcctcacct ccaaataaat |
| 5881 | atgtgtgtgt ctgtgtgtgt gtatatatgt atgtggcgga gagggagaga gtggggaagg |
| 5941 | agagcagtgt tatcatacat agagaggcta aatgtgtccc atccctcact gtcagcttta |
| 6001 | taaaggagtt tgactccatc cacagaagaa tgttttataa gactaggaaa acacgttgaa |
| 6061 | aactaggata aacagcaaca aaaatcaact aaatatgttg ttactgttgc taaggatttt |
| 6121 | ctccttagaa taatttagga tttttaaaaa tttctgtttg ccaaatgctg tagataaatg |
| 6181 | gccagattct tcctatccct aggattcctt tattattttt tttcacagat tttgagaaca |
| 6241 | agggggagag atagtatgga agattaagat tccattaatc ttatagaact gtgttgtcac |
| 6301 | ccaaattcct gcttgtttga acatggcatc ttcatagatt caggattcac taccctctat |
| 6361 | agctggatct tgaaaattat ctggccagat aattttgcat ctgcttggat gattgtagac |
| 6421 | tgagatgtga gtggaggata aagtattaga cttttgctga gtaactgcca accaagaagt |
| 6481 | atttatcgga cacttactag gtgcctagga ttgtatcaga gggaatatga aatgtgtccc |
| 6541 | tgccctacct agttttaacg acagaatatc tattaaaggc tacttagctg aagggtaagg |
| 6601 | gtgacaggtc taggggaagc tttgggaggt ggtgtgctgt gacagaaaaa gtggcagagt |
| 6661 | agggacgaga gacctgcatt ctagccctgt ttctgtcact tgctctgtac acttagacaa |
| 6721 | cagcttgacc tcttgagctt tagtttcctc ctctgcataa tgagagggtt agactactga |
| 6781 | attgtatggg aaaaaaatac aaattcctgg gtcctaggcc atgcctgctg aatccgactg |
| 6841 | ttcaggaaga ggcctaggaa atctgtgagg gaatccccag gggaatctcg tgaccagcca |
| 6901 | ggtgtgaaat ctgctaactg gaagatctca aagcttcctt cactttttgt gattttgtgg |
| 6961 | tcatgtaacg ttactgtatt attctacgta aatgtgggta cttggatgtt tatcatactg |
| 7021 | tttctctgtg tttacatact aatttgtgta agaaatgcat tttagtctgt gtacctcaac |
| 7081 | ctgctgtttg tttcctagag gtgttagtag tctttaaata caagtaagac ttaagaggat |
| 7141 | atttgatgtt atttacctgg atattttctt ccccttttat ttatttagag gaaattgaga |
| 7201 | ttctaggagc caaaaaaatg aaaacaaaat tctaaggcaa agttaaagaa aaaaattaca |
| 7261 | ttatttctta ccatttgcta ctttataatg aaaatttaaa aattatatgg gaagattttt |
| 7321 | ctctgggata acaaatcctt gtcataaagt aagaggtctt tttaaagtag gtaggctata |
| 7381 | aggcctgtaa tttaaaataa tactcctttc tctagggttt ggtgcaattc tccattaatg |
| 7441 | aagataacat ttgaattccc caaagcaggt gaggagtcgg ggaggagaaa gcgatgttaa |
| 7501 | aatgaaaact cactgcaaaa gaggaggcag aggaagaagg aatgtaaacc ccttaaagca |
| 7561 | gatgtgtgtg gggccttatg aagaccagga ttctgcgggt gtcaggggat tgcccctctt |
| 7621 | gacagagact agggttttag actgaggctt cctgcagggt gttcgcattg cccttctccg |
| 7681 | ttccccttca gacctttctg gggagaagag gtgggaggag gggagaaaga ctgttcatct |
| 7741 | tattctgaat cctggagcag ctgaaggttt tctcttgagt caggatgcag tggtaatgca |
| 7801 | ttaaccagca agtgtggcca aggataatga aaaagtggga aaggaaggtc ctcctcctcc |
| 7861 | ctgattgtag catccagcag tctctgtagc caggttactc aagaaccaca tttgatttcc |
| 7921 | tggccctttg ccttggcagt gatggcattt ttatttcact gtgttttaaa gtcttcattt |
| 7981 | atttttataa catgggttag ggagaagggc cacaaatgga gggattgtcc tttcaagcac |
| 8041 | cacagcttca gataaaatta gtactttcaa atattgtcca ctttaactta aaaaattcta |
| 8101 | gagggattat attggagact caactgccct tggttttagt ttataaaatg gcctagtact |
| 8161 | gtggaatttt aattttagaa agtcttagca tcagatcata aacattcatt aaaagaactc |
| 8221 | acatcccatc tgaaacttcc caggggagtt gggattctta gtagattggt agaaaggggc |
| 8281 | tcattttcta ctgcatttcc catttttggt atcttgttca gcatgtttta tttttatttc |
| 8341 | ttgtctgcag aacatcctat atttatgaga acattcttta agaagaccac cacatagaat |
| 8401 | accccttcct atcagctcgc tctgatttag ccttaatttt gttaaatttt ttagagatga |
| 8461 | atgaagtgct gctgtggaaa gaaatgtaca tatactattt ctgtatcatt aaaattacat |
| 8521 | ttttatggtt c |

SEQ ID NO: 40 Human CYLD transcript variant 3 cDNA sequence
(NM_001042412,2; CDS: 335. . . 3196)

| | |
|---|---|
| 1 | aggtagcagg tttggctgcg cgggggccgc gcgtcggagg taaatactag ggcggtgggt |
| 61 | gtggggagcc ggggccggcc cgggacgcgg gctggggagc cggggcgagg ggcgacgccc |
| 121 | cgccgcccga gtttccccct ttctagggtg aggatggttc tacacagcca cccggagttc |
| 181 | cttagttgaa aggtgcgccc tgctgtgaca gcatggacac cacgttgctg aaaacatgct |
| 241 | ttgggactgc cactgaattt atcttttgcg gttttatgac aaagttatta gtagtttccc |
| 301 | ttttttgaat tagtattttg aagttaatat cacaatgagt tcaggcttat ggagccaaga |
| 361 | aaaagtcact tcaccctact gggaagagcg gattttttac ttgcttcttc aagaatgcag |
| 421 | cgttacagac aaacaaacac aaaagctcct taaagtaccg aagggaagta taggacagta |
| 481 | tattcaagat cgttctgtgg ggcattcaag gattccttct gcaaaaggca agaaaaatca |
| 541 | gattggatta aaaattctag agcaacctca tgcagttctc tttgttgatg aaaaggatgt |
| 601 | tgtagagata aatgaaaagt tcacagagtt acttttggca attaccaatt gtgaggagag |
| 661 | gttcagcctg tttaaaaaca gaaacagact aagtaaaggc ctccaaatag acgtgggctg |
| 721 | tcctgtgaaa gtacagctga gatctgggga agaaaaattt cctggagttg tacgcttcag |
| 781 | aggacccctg ttagcagaga ggacagtctc cggaatattc tttggagttg aattgctgga |
| 841 | agaaggtcgt ggtcaaggtt tcactgacgg ggtgtaccaa gggaaacagc tttttcagtg |
| 901 | tgatgaagat tgtggcgtgt ttgttgcatt ggacaagcta gaactcatag aagatgatga |
| 961 | cactgcattg gaaagtgatt acgcaggtcc tggggacaca atgcaggtcg aacttcctcc |
| 1021 | tttggaaata aactccagag tttctttgaa ggttggagaa acaatagaat ctggaacagt |
| 1081 | tatattctgt gatgttttgc caggaaaaga aagcttagga tattttgttg gtgtggacat |
| 1141 | ggataaccct attggcaact gggatggaag atttgatgga gtgcagcttt gtagttttgc |
| 1201 | gtgtgttgaa agtacaattc tattgcacat caatgatatc atcccagaga gtgtgacgca |
| 1261 | ggaaaggagg cctcccaaac ttgcctttat gtcaagaggt gttggggaca aaggttcatc |
| 1321 | cagtcataat aaaccaaagg ctacaggatc tacctcagac cctggaaata gaaacagatc |
| 1381 | tgaattattt tataccttaa atgggtcttc tgttgactca caaccacaat ccaaatcaaa |
| 1441 | aaatacatgg tacattgatg aagttgcaga agaccctgca aaatctctta cagagatatc |
| 1501 | tacagacttt gaccgttctt caccaccact ccagcctcct cctgtgaact cactgaccac |
| 1561 | cgagaacaga ttccactctt taccattcag tctcaccaag atgcccaata ccaatggaag |
| 1621 | tattggccac agtccacttt ctctgtcagc ccagtctgta atggaagagc taaacactgc |
| 1681 | acccgtccaa gagagtccac ccttggccat gcctcctggg aactcacatg gtctagaagt |
| 1741 | gggctcattg gctgaagtta aggagaaccc tcctttctat ggggtaatcc gttggatcgg |
| 1801 | tcagccacca ggactgaatg aagtgctcgc tggactggaa ctggaagatg agtgtgcagg |
| 1861 | ctgtacggat ggaaccttca gaggcactcg gtatttcacc tgtgccctga agaaggcgct |
| 1921 | gtttgtgaaa ctgaagagct gcaggcctga ctctaggttt gcatcattgc agccggtttc |
| 1981 | caatcagatt gagcgctgta actctttagc atttggaggc tacttaagtg aagtagtaga |
| 2041 | agaaaatact ccaccaaaaa tggaaaaaga aggcttggag ataatgattg ggaagaagaa |
| 2101 | aggcatccag ggtcattaca attcttgtta cttagactca accttattct gcttatttgc |
| 2161 | ttttagttct gttctggaca ctgtgttact tagacccaaa gaaaagaacg atgtagaata |
| 2221 | ttatagtgaa acccaagagc tactgaggac agaaattgtt aatcctctga gaatatatgg |
| 2281 | atatgtgtgt gccacaaaaa ttatgaaact gaggaaaata cttgaaaagg tggaggctgc |
| 2341 | atcaggattt acctctgaag aaaaagatcc tgaggaattc ttgaatattc tgtttcatca |
| 2401 | tattttaagg gtagaacctt tgctaaaaat aagatcagca ggtcaaaagg tacaagattg |
| 2461 | ttacttctat caaattttta tggaaaaaaa tgagaaagtt ggcgttccca caattcagca |
| 2521 | gttgttagaa tggtctttta tcaacagtaa cctgaaattt gcagaggcac catcatgtct |
| 2581 | gattattcag atgcctcgat ttggaaaaga ctttaaacta tttaaaaaaa tttttccttc |
| 2641 | tctggaatta aatataacag atttacttga agacactccc agacagtgcc ggatatgtgg |
| 2701 | agggcttgca atgtatgagt gtagagaatg ctacgacgat ccggacatct cagctggaaa |
| 2761 | aatcaagcag ttttgtaaaa cctgcaacac tcaagtccac cttcatccga agaggctgaa |
| 2821 | tcataaatat aacccagtgt cacttcccaa agacttaccc gactgggact ggagacacgg |
| 2881 | ctgcatccct tgccagaata tggagttatt tgctgttctc tgcatagaaa caagccacta |
| 2941 | tgttgctttt gtgaagtatg ggaaggacga ttctgcctgg ctcttctttg acagcatggc |
| 3001 | cgatcgggat ggtggtcaga atggcttcaa cattcctcaa gtcaccccat gcccagaagt |
| 3061 | aggagagtac ttgaagatgt ctctggaaga cctgcattcc ttggactcca ggagaatcca |
| 3121 | aggctgtgca cgaagactgc tttgtgatgc atatatgtgc atgtaccaga gtccaacaat |
| 3181 | gagtttgtac aaataactgg ggtcatcggg aaaggcaaag aaactgaagg cagagtccta |
| 3241 | acgttgcatc ttattcgagc tggcagttct gttcacgtcc attgccggca atggatgtct |
| 3301 | ttgtggtgat gatccttcag aaaaggatgc ctctgtttaa aaacaaattg cttttgtgtc |
| 3361 | cctgaagtat ttaataagaa gcattttgca ctctagaaag tatgtttgtg ttggtttttt |
| 3421 | aagaagtcta aatgaagtta ttaatacctg aagctttaag ttaagtgcat tgatcatatg |
| 3481 | atatttttgg aagcatacaa ttttaattgt ggaagtttaa agcctctttt agtccattga |
| 3541 | gaatgtaaat aaatgtgtct tctttatgga ccaaggatat gaaatcattt ttcttttgta |
| 3601 | gctaacggtt gccttgagga agaaataatt tggttttatt aagagtctac tctcaatcca |
| 3661 | gttattagag atgtactgag tttgatttgt taatcctttc tatatactgc tgatcttgca |
| 3721 | tgtctacaat ctgctcagtt tttctgtgtt tctgcaatag tggtcagaaa aatacttaaa |
| 3781 | ttcccttaat ggtgttgttt tctatttgtt ctggttttga gataaatgag tgattctgtc |
| 3841 | cccaaatgtc catttttgaa gtgattttcc tggaggatta gggtatttag cagttgaagc |
| 3901 | tcttcattca tagtagttac tgtcagctaa caggtttttt aaggctttta actattaata |
| 3961 | ttttatggaa tggggcaaag taaattgatg aaagaattgg agtgataata gtcctttaca |
| 4021 | aacatacagt ccataagaaa atgaatttgg catatagaat tattacaatt tcctgggaga |
| 4081 | gatggatatt taaacctcta ttattttaga caagactgtc tagaacttaa gtttgatctg |
| 4141 | tcagccagta ctcccattaa attcagtgta gtttcacttg atagaatcag atatgttatc |
| 4201 | gaaatgttag cagcagcttc atcctccttc tgattaaagt aagtagaaat gggatgtttt |
| 4261 | gtttaataac agccatagtg tgtgtttaga ccacagcgga tgttgtagac caggaccata |
| 4321 | gatgatacat gtcagtgctg tggaatgtgc attctctgag tgttgttttg tggtatcatt |
| 4381 | gtctttcctg aatgactttc taactgtgca gaaaggcaga aaagtcatca tatgtatatg |
| 4441 | tcatatgact ttataaaata tttaatgtga caaaaagtgg aaagaatctt tacaaaccct |
| 4501 | gcaattactt ttttaaaggc acttttactc tttggtttta tcattccatt ttgctaatat |
| 4561 | ttactagctt tataaattac agtaaggtac aaaaactcat cttgtaatat tttcattttt |
| 4621 | gaagtgaaaa agtacatata ttttgcacaa ggttttatac tgctaagtgc ttggttgggg |
| 4681 | tggtgagatg atgattagat caggggtgag gctgagagac tctgggttta gggctagccc |
| 4741 | tgcctccatc tcccttgggt aaaatgaagg gtgtggggta aaagatgcat aaggcctttt |
| 4801 | ctagctctga cagcctagaa gtccaatcac cctgtaataa atatgtgttg aatgaagaaa |
| 4861 | tgggtgaatg agcttgtcaa tgtgatttta aaaaattgac tacctggagg aatgattagg |
| 4921 | aatctaaatg aagccagccc tcggtatctg caggtttctc atccatggat tcaaccaact |
| 4981 | gcaaatggaa aatacgattt tttttaaaaa aaggatggtt acatccgtat tgaacatgta |
| 5041 | cagacttttt tcttgtcatt attctctgaa caatacaaga actctttatg tagcatttac |
| 5101 | atttattagg tattataagt aatctagaga ttatttaatt aaaatataca ggaggatgtg |
| 5161 | tgtcagttat atgcaaattc tgtaccattt tgtatcaggg aattgagcat cttcagatgt |
| 5221 | tggtatctgc agggatcctg gaaccaaacc cctgcagata ctaagggctg acgatctagg |
| 5281 | taagactgga tttaacagtt ggaaaaaaaa aaaaaaaagg agagagaaga cagttccttt |
| 5341 | cctgtagaaa ttaaaacaaa atacaaattg aggaagctct gctacccagg ctgtcatggt |
| 5401 | agagaacttg aagaagacct gtttggatgg acacctggtt tcaaaagtca ggtgtggaga |
| 5461 | ctgttaaatg ggagggcctc atccataaat gatttctggc aacgtcttct tcaggtggag |
| 5521 | cttgacgtct ttttaatgtt acttggggag ggagtgctca ttaagggatg ccagggccag |
| 5581 | ctctggtggt tcctggggag gctgcgtcct tccctgcttc tgcatgtcat gaggcagcag |
| 5641 | gaaggtttcc cctgcacctg tctgtcctgg ctccctctgg gtagccccct actgttctgt |
| 5701 | gcttcagcac agcctggttt gtcaagaggc acatagttgg ggctgggctg catggcacag |
| 5761 | gggcttatgt gcctgctggt tatttaattt tcagccttaa gttttcttta atattttcct |
| 5821 | gttggctatt taaaggtttt ggttatcttt tattccttat ctacaatcaa gatgacaatg |
| 5881 | taattgaatt atcttattta taacacggtt cgtgattcat gattcatgat tacaagtaga |
| 5941 | aaatatgtca tgttcctcac ctccaaataa atatgtgtgt gtctgtgtgt gtgtatatat |
| 6001 | gtatgtggcg gagagggaga gagtggggaa ggagagcagt gttatcatac atagagaggc |
| 6061 | taaatgtgtc ccatccctca ctgtcagctt tataaaggag tttgactcca tccacagaag |
| 6121 | aatgttttat aagactagga aaacacgttg aaaactagga taaacagcaa caaaaatcaa |
| 6181 | ctaaatatgt tgttactgtt gctaaggatt ttctccttag aataatttag gatttttaaa |
| 6241 | aatttctgtt tgccaaatgc tgtagataaa tggccagatt cttcctatcc ctaggattcc |
| 6301 | tttattattt tttttcacag attttgagaa caagggggag agatagtatg gaagattaag |
| 6361 | attccattaa tcttatagaa ctgtgttgtc acccaaattc ctgcttgttt gaacatggca |
| 6421 | tcttcataga ttcaggattc actaccctct atagctggat cttgaaaatt atctggccag |
| 6481 | ataattttgc atctgcttgg atgattgtag actgagatgt gagtggagga taaagtatta |
| 6541 | gacttttgct gagtaactgc caaccaagaa gtatttatcg gacacttact aggtgcctag |
| 6601 | gattgtatca gagggaatat gaaatgtgtc cctgccctac ctagttttaa cgacagaata |
| 6661 | tctattaaag gctacttagc tgaagggtaa gggtgacagg tctaggggaa gctttgggag |
| 6721 | gtggtgtgct gtgacagaaa aagtggcaga gtagggacga gagacctgca ttctagccct |
| 6781 | gtttctgtca cttgctctgt acacttagac aacagcttga cctcttgagc tttagtttcc |
| 6841 | tcctctgcat aatgagaggg ttagactact gaattgtatg ggaaaaaaat acaaattcct |
| 6901 | gggtcctagg ccatgcctgc tgaatccgac tgttcaggaa gaggcctagg aaatctgtga |
| 6961 | gggaatcccc aggggaatct cgtgaccagc caggtgtgaa atctgctaac tggaagatct |
| 7021 | caaagcttcc ttcacttttt gtgattttgt ggtcatgtaa cgttactgta ttattctacg |
| 7081 | taaatgtggg tacttggatg tttatcatac tgtttctctg tgtttacata ctaatttgtg |
| 7141 | taagaaatgc attttagtct gtgtacctca acctgctgtt tgtttcctag aggtgttagt |
| 7201 | agtctttaaa tacaagtaag acttaagagg atatttgatg ttatttacct ggatattttc |
| 7261 | ttcccctttt atttatttag aggaaattga gattctagga gccaaaaaaa tgaaaacaaa |
| 7321 | attctaaggc aaagttaaag aaaaaaatta cattatttct taccatttgc tactttataa |
| 7381 | tgaaaattta aaaattatat gggaagattt ttctctggga taacaaatcc ttgtcataaa |
| 7441 | gtaagaggtc tttttaaagt aggtaggcta taaggcctgt aatttaaaat aatactcctt |
| 7501 | tctctagggt ttggtgcaat tctccattaa tgaagataac atttgaattc cccaaagcag |
| 7561 | gtgaggagtc ggggaggaga aagcgatgtt aaaatgaaaa ctcactgcaa aagaggaggc |
| 7621 | agaggaagaa ggaatgtaaa ccccttaaag cagatgtgtg tggggcctta tgaagaccag |
| 7681 | gattctgcgg gtgtcagggg attgcccctc ttgacagaga ctagggtttt agactgaggc |
| 7741 | ttcctgcagg gtgttcgcat tgcccttctc cgttcccctt cagacctttc tggggagaag |
| 7801 | aggtgggagg aggggagaaa gactgttcat cttattctga atcctggagc agctgaaggt |
| 7861 | tttctcttga gtcaggatgc agtggtaatg cattaaccag caagtgtggc caaggataat |
| 7921 | gaaaaagtgg gaaaggaagg tcctcctcct ccctgattgt agcatccagc agtctctgta |
| 7981 | gccaggttac tcaagaacca catttgattt cctggccctt tgccttggca gtgatggcat |
| 8041 | ttttatttca ctgtgtttta aagtcttcat ttatttttat aacatgggtt agggagaagg |
| 8101 | gccacaaatg gagggattgt cctttcaagc accacagctt cagataaaat tagtactttc |
| 8161 | aaatattgtc cactttaact taaaaaattc tagagggatt atattggaga ctcaactgcc |
| 8221 | cttggtttta gtttataaaa tggcctagta ctgtggaatt ttaattttag aaagtcttag |
| 8281 | catcagatca taaacattca ttaaaagaac tcacatccca tctgaaactt cccaggggag |
| 8341 | ttgggattct tagtagattg gtagaaaggg gctcattttc tactgcattt cccatttttg |
| 8401 | gtatcttgtt cagcatgttt tatttttatt tcttgtctgc agaacatcct atatttatga |
| 8461 | gaacattctt taagaagacc accacataga ataccccttc ctatcagctc gctctgattt |
| 8521 | agccttaatt ttgttaaatt ttttagagat gaatgaagtg ctgctgtgga aagaaatgta |
| 8581 | catatactat ttctgtatca ttaaaattac atttttatgg ttc |

SEQ ID NO: 41 Mouse CYLD Isoform 1 Amino Acid Sequence (NP_001121642.1)

| | |
|---|---|
| 1 | mssglwsqek vtspyweeri fylllqecsv tdkqtqkllk vpkgsigqyi qdrsvghsrv |
| 61 | pstkgkknqi glkileqpha vlfvdekdvv einekftell laitnceerl slfrnrlrls |
| 121 | kglqvdvgsp vkvqlrsgee kfpgvvrfrg pllaertvsg iffgvellee grgqgftdgv |
| 181 | yqqkqlfqcd edcgvfvald kleliedddn glesdfagpg dtmqvepppl einsrvslkv |
| 241 | gestesgtvi fedvlpgkes lgyfvgvdmd npignwdgrf dgvqlcsfas vestillhin |
| 301 | diipalsdsv tqerrppkla fmsrgvgdkg ssshnkpkvt gstsdpgsrn rselfytlng |
| 361 | ssvdsqqsks knpwyideva edpaksltem ssdfghsspp pqppsmnsls senrfhslpf |
| 421 | sltkmpntng smahsplsls vqsvmgelns tpvqespplp issgnahgle vgslaevken |
| 481 | ppfygvirwi gqppglsdvl agleledeca gctdgtfrgt ryftcalkka lfvklkscrp |
| 541 | dsrfaslqpv snqiercnsl afggylsevv eentppkmek egleimigkk kgiqghynsc |
| 601 | yldstlfclf afssaldtvl lrpkekndie yysetqellr teivnplriy gyvcatkimk |
| 661 | lrkilekvea asgftseekd peeflnilfh dilrvepllk irsagqkvqd cnfyqifmek |
| 721 | nekvgvptiq qllewsfins nlkfaeapsc liiqmprfgk dfklfkkifp slelnitdll |
| 781 | edtprqcric gglamyecre cyddpdisag kikqfcktcs tqvhlhprrl nhsyhpvslp |
| 841 | kdlpdwdwrh gcipcqkmel favlcietsh yvafvkygkd dsawlffdsm adrdggqngf |
| 901 | nipqvtpcpe vgeylkmsle dlhsldsrri qgcarrllcd aymcmyqspt mslyk |

SEQ ID NO: 42 Mouse CYLD transcript variant 1 cDNA sequence
(NM_001128170.2; CDS: 223 . . . 3090)

| | |
|---|---|
| 1 | agttcggagg cggggcaggt gggggcgggc ccaggtagca ggttcggctg cgcgggggcc |
| 61 | cgcgcgcctg aggcactttg aattgctgtc tttttacaac atggatgcca ggttgctaaa |
| 121 | atcttgcttt gggacttaca gcgagttcat ttttttggat tttatgacag atttactagt |
| 181 | ggctcctttt tgccagtcaa tgttctgaaa gttactgtca caatgagttc aggcctgtgg |
| 241 | agccaagaga aagttacttc accctactgg gaagaacgga ttttttatct gcttcttcaa |
| 301 | gaatgcagtg taacagacaa acaaactcag aagctgctga aagtacccaa agggagtata |
| 361 | ggacagtaca tccaagaccg ttctgtgggg cattcaagag ttccttccac aaaaggcaag |
| 421 | aaaaatcaga ttggattaaa aatcttggag caaccgcatg cagttctgtt tgttgatgaa |
| 481 | aaggatgttg tagaaataaa tgaaaaattc acagagttac tgttggcaat taccaactgt |
| 541 | gaggagaggc tcagcctatt tagaaacaga ctccgactaa gtaaaggcct ccaggtagac |
| 601 | gtgggcagtc ctgtgaaagt acagctgcga tctggggaag agaaatttcc aggagttgta |
| 661 | cgcttcagag gacctttatt agcggagagg acggtgtcgg ggattttctt tggagtagaa |
| 721 | ttattggaag aaggtcgtgg tcaaggtttc acggatgggg tatatcaagg gaagcagctt |
| 781 | ttccagtgtg atgaggactg tggcgtgttt gttgcattgg acaaactaga acttatagaa |
| 841 | gatgatgaca atggattgga aagtgatttt gcaggcccag gagatacaat gcaggttgaa |
| 901 | cctccccctt tggaaataaa ctccagagtt tctttgaagg ttggagaaag tacagaatct |
| 961 | ggaacagtaa tattctgtga tgttttacca ggaaaagaga gtctaggata ttttgttggt |
| 1021 | gtggacatgg ataaccctat tggcaactgg gatggaaggt ttgatggagt acagctctgt |
| 1081 | agttttgcaa gtgttgaaag tacaattctc ctgcacatca atgacatcat cccagcttta |
| 1141 | tcagatagcg tgacacagga aaggaggcct cccaaacttg cctttatgtc aagaggtgta |
| 1201 | ggtgacaaag gttcatctag tcataataaa ccaaaggtta caggatctac ctcagaccct |
| 1261 | ggaagtagaa acagatctga attattttat accttaaatg ggtcatctgt tgactcacaa |
| 1321 | caatccaagt ccaaaaatcc atggtacatt gatgaagttg cagaagaccc tgcaaagtca |
| 1381 | cttacagaga tgtcttcgga cttcggacat tcatctcctc caccgcagcc tccttccatg |
| 1441 | aactccttgt ctagcgagaa cagattccac tccttaccct tcagcctgac aaagatgccc |
| 1501 | aatactaatg gcagcatggc tcatagtcca ctctctctgt cagtgcagtc tgtgatgggg |
| 1561 | gagctgaaca gcacacctgt ccaggagagt ccacccttgc ccatctcttc tgggaatgca |
| 1621 | cacgggctag aggtgggctc actggctgaa gtaaaagaga accccccgtt ctatggggtt |
| 1681 | atccgttgga ttggccagcc accagggctc agtgacgtgc tagctggact ggaactggaa |
| 1741 | gatgaatgcg caggctgtac agatggaact ttcaggggca cgcggtattt cacgtgtgcc |
| 1801 | ctgaagaagg cactgtttgt gaaactgaag agctgcagac cggactctag gtttgcatcc |
| 1861 | ttgcagcctg tttccaatca aattgaaagg tgtaactctt tagcatttgg gggctattta |
| 1921 | agtgaagtag tagaagaaaa tactccacct aaaatggaaa aggaaggttt agagataatg |
| 1981 | attggaaaga agaaaggcat ccagggccat tacaattctt gttacttaga ctcaacttta |
| 2041 | ttctgcttat ttgcttttag ttctgccctg gacactgtgt tacttagacc caaagagaag |
| 2101 | aatgatatag agtattacag tgagactcag gagctactga ggacagagat agtcaatcct |
| 2161 | ctgagaatat atggatatgt gtgtgccaca aagattatga aactgaggaa aatacttgaa |
| 2221 | aaagttgagg ctgcatcagg ttttacctct gaagaaaaag atcctgaaga attcttaaat |
| 2281 | atcctgtttc atgatatttt aagggttgaa ccattgttaa aaataagatc agcaggtcaa |
| 2341 | aaagttcaag actgtaactt ctatcaaatt tttatggaaa aaaatgagaa agttggagta |
| 2401 | cccacaattc agcagttatt agaatggtct tttatcaaca gcaacctgaa atttgcagag |
| 2461 | gcaccatcat gcttgattat ccagatgcct cggtttggaa aagactttaa actatttaaa |
| 2521 | aaaatttttc cttccctgga attaaatata acagatttac ttgaagacac tcccaggcag |
| 2581 | tgccgcatct gtggaggact tgcgatgtac gagtgcaggg agtgctatga cgatccggac |
| 2641 | atctcagctg ggaagatcaa gcagttctgt aagacctgca gcactcaggt tcaccttcat |
| 2701 | cccaggaggt tgaatcattc ttatcatcca gtatcacttc ccaaagactt gcctgactgg |
| 2761 | gactggagac acggctgcat cccctgtcag aagatggagt tatttgctgt tctctgcata |
| 2821 | gaaacgagcc actatgttgc ttttgtgaag tatgggaagg atgactctgc ctggcttttc |
| 2881 | tttgacagca tggcggatcg agatggtggt cagaatggct tcaacattcc acaagtgacg |
| 2941 | ccctgcccag aagtgggaga gtacttgaag atgtctctgg aggacctgca ctctttggat |
| 3001 | tccagaagga ttcaaggctg tgcgcgcaga cttctttgcg atgcatacat gtgcatgtac |
| 3061 | cagagtccaa ccatgagcct gtacaaataa ccaggttatc cgcagtgggc agagacagca |
| 3121 | caaaccttag tgctgtgtct tcctccagct tgtggttctg tccggcaccc attgccggca |
| 3181 | atgggtgtct cttttgatga tggcccttca gcaaaggatt cctgtgtttc caaacaaatt |
| 3241 | gcttttgtgt tcctgaagta tttaataaga agcattttgc actctaggaa gtatgtttgt |
| 3301 | gttgattttt taagaagtct aaatggattt attaatatct gacactttaa gttaactgca |
| 3361 | ttgatgatac gatatttttg gaagcataca attttaattg tgacgtctaa aacctctttt |
| 3421 | agtccattga ggatgtaaat aaatatttct tcttatggac cagggatatg aaatcatttt |
| 3481 | ccttttgtag ctaatgattg ccttgagaaa gacataatat gattttatta agagtctgcc |
| 3541 | tttactccaa ttgttgaagg tgtgctgagt ttaattgtta attttcctat atagtgctga |
| 3601 | tttcctgcat gtctgtactt tgctgagttt ctgtgttcct gcagcagtgg tcagaacatg |
| 3661 | ctgaaattcc tttcatggtg ttatttttta tttgttctag ttttgagata aatgagtgac |
| 3721 | tctgtcataa aatgtccatt tttgatttgt tcttgaagaa ttagggtaca aaacaagtat |
| 3781 | cgtagtcata gtaacaactg ttactgaaca ggtcttctgt aggggctcct aagtatgaga |
| 3841 | caacatgcag tggtttctga aatgactgag cgataagtaa attatatatt ctttttttaa |
| 3901 | aagatttatt ttatttatat gagtacactg tagctgtctt cagacacacc agaagagggc |
| 3961 | atcagatctt attacagatg gttgtgagcc accatgtggt tgctgggaat tgaactcagg |
| 4021 | acctttggaa gggcagtcag tgctcttaac tgctgagcta tctctctagc cccaaattat |
| 4081 | atattcttat acagacccac agtccagaga aagtgaagct ggctcagagt tagcaccaac |
| 4141 | atttccctga aaaaatgtgt taggtgttta aaaatctcat cgtaaataaa ttattttatt |
| 4201 | tttagtttaa gtgaatttct cttaatttaa ataagactaa tatttttgtt tgactgaaga |
| 4261 | cccttgtact ctctggtgtc agtttggtgt cacttgaaac aatgacaacc acgtcagcat |
| 4321 | gttactgcaa tgtcagtgat ggtttcaaag gagaaaatgg atgtcatctt aggactgctt |
| 4381 | gtgtgtgtat agaccgtggt ggatgctgta gagtgcttgt tgtctgagtg ttgttttgtg |
| 4441 | gtatggctgt ctctctgaat aatgtcaacc ttacagaaag agaaggtgtt atatgtgtaa |
| 4501 | catatggctt tataaaagat ttaatgtcaa aagagtctat aaatattgct gttattttta |
| 4561 | aaggcacttt actccttggt tttattattc cattttgctg gcgtgtatta gctttatata |
| 4621 | aatcacaaca cacaaaactc gtccatcttg taatctttgt acttttgcaa tggaaaagtg |
| 4681 | tgtatactgt gcacagactt ttatatagct gagtgcctag actgggtgga gaggccatga |
| 4741 | gtagatagca gtaaagtcct ccagctatag gctagccttg tcttcatcat cctgggacag |
| 4801 | gtgagaacgg tgagcacagg tgagtctgag cttgcacact gaccagaatt cagtcaccct |
| 4861 | gtaataagta tgtatttaat gagtctgtta gtgtggtagt gtgattttga aaggtttaac |
| 4921 | taaggaatgg tgaggtcttt gtttataggc aagcctggct taacagtgaa ggggaggggg |
| 4981 | ctgagggaaa gctttttttt tttttaattt cagaaatgga agcaaaatcc atgtgcaaag |
| 5041 | agactagcta cttggctttc agagcagagg actcaaagct tgtttgcagt ggttctgaag |
| 5101 | ttggggtggc actttgtgag tgaggaggac catgtccaca ggcaatccct aagcaagatc |
| 5161 | gtctcgaaat tgcactgtgt tctttagcag tagtggctgg gtaggggcac acagcggaac |
| 5221 | acccacttgc agtggctggg gaggcctgag aagcacaggc cagcgggagc tctctcccat |
| 5281 | gccttcttgt ccctctggac agcccctcct cattccttgt cccagcagag cctggtttgc |
| 5341 | ccctggggca atagggttgg actgtgtggt gcagatagcc ctcgtgcctg tggattactt |
| 5401 | taattttgta atcttaggtt ttctgtacgg ttttcccatt gccttttaaa atattttgag |
| 5461 | tgtccttctc cttatcaaga tggtagtgta gtaggattgt tttactcaca acatggttta |
| 5521 | ggagccatag ctacaaatag gaaatataag aattctattt ctctactgtg aggcaaatct |
| 5581 | gtatatatgt atggagagtc aggaggatga acaagggaga ggaaaggggc ccaggcacat |
| 5641 | ttttaatcca aaagctacaa agtgtcgagc tcctcagggc cagcttcaca gagagtccag |
| 5701 | ctcagttcac aaagtatttt ctaagttggg gaaatgcatt gagaaacaga gcgagatatc |
| 5761 | tgtgtagtgc agctgtgggt ggttgctatg cattttatcc tcagagtagc ctggggtttt |
| 5821 | tattttatgt ttgccaagtg cttaggtaaa tggccgggct cctcttacct ctgacttctg |
| 5881 | gagcgtgtcc attctctggt tgactgaatg tggcattttc ataaatctag gagcagcagc |
| 5941 | cctcccaact gcaatttagt aagcaggtag ataagcttgc atctgtttgc atcttcctgc |
| 6001 | actgagttgg gagtgcaaaa ccccgtactt ggcctggtga gtaactgcca acaagtattt |
| 6061 | atgggtcatt tgctaggtgg ctagcatacg gcacggggct aatgagacct ggggcctgct |
| 6121 | ctcatatagc ttactgctca gtttctatac cagagcatcc gttggtgctt cttagcctag |
| 6181 | gcatgggtga caggcatgtc tggggaccac tttgggagac aacctagtgt aaccttattg |
| 6241 | caaaggacta agagacaaaa atcctgggtt ctttctccat cacttgctct gtagctgaat |
| 6301 | gaacaccacc tcgatttctt gagtttcagt gtcttgttct gtctgatgaa agggttggat |
| 6361 | atgtagttgg ataagcggta caaattgtta gggttccagg ccatgcctga tacatctgag |
| 6421 | tttttgctgg aggtaggaaa tttcaactca gaagctcatc cacagaaaac agtgaccagc |
| 6481 | agcaggtatg gagccataag gctggaaggc ttcaaagacc ccttcttctg ttactgttat |
| 6541 | ccacggcagt tattctgtgt aggtgtgaag gagtggctac tgactctgtg tggatcagcg |
| 6601 | tttacatatg acctgatggg agtgcatctg gtatgtgttc ctcccccctg tattgctgga |
| 6661 | gagattggca atcgtaggta tacacggaag tgtcgggatg ttcagtggcg cgcacttgga |
| 6721 | tgtttctttt cactttattt ctatagaaac aggttccagg agccagagaa gtaggggaaa |
| 6781 | aaatccctga gctcatctct ctctactact taataatgca aacatttaaa ttcttgggaa |
| 6841 | gattttcccg ccccactccc tacccccagt cacaggtcag aataggattt ccttcttaga |
| 6901 | gaaggtaggt catggggcct gcgattaaaa aaacaaaaac ataaaacaaa caaacaaaca |
| 6961 | aggtggcttt ccttggtctt tgtgattcca gctcttcacc agtgaaggaa ttctttagtt |
| 7021 | ccatagtgca gcctgggtgg gtggagaaga ggctgttata gtgggagcca gtgagcaaag |
| 7081 | agggttggag gaggcagagg acagggctca gggaaggcaa gcctgtggct ctgggcattg |
| 7141 | gccctcttgt caaaggtgtc agagcatggc ttcctgtgag gggctttccc ttgctcctgt |
| 7201 | gtgagaatct gtgcgttgtc ttactggatg ggggacctgg atggagatgt taggggagat |
| 7261 | agcttagtct tgttctaaac tctagacttg ctaaaaaggg ttctcttgag tccaaatgca |
| 7321 | gtgccagagg gtgagaagca gaattggcca gtagtgaaca gtggtattgt gggtgataag |
| 7381 | ccctttctgg tggccttaaa tagcagtgct agtccttgag tgccacaggg tcccctacct |
| 7441 | ccgtcctggc agtgctgatg ggtttgtttc agcatatttt ggttctcatt ctcatttagg |
| 7501 | gaaaagggct gttgatgaag ggattatcct cccaggcacc aggggcctca gcacagttcc |
| 7561 | ctgtgctttt aaacactgtt gactttcatt agttattcta aagggattta tattggagat |
| 7621 | acaactccct ttggtttcat attatgaaat agctttgtac tgtggaatgc taactctgga |
| 7681 | aagtctccct cataccagaa ccaaacattc atttctttta tatatatata tatattatct |
| 7741 | ccctggtact ctccagggag gtggttatta tcaagctgac ccaaaggagt gcagtatgtg |
| 7801 | ttctagttcc ttttctggta ccttgttcaa cattcttttt tcctgaagaa catccttttg |
| 7861 | tttatgaaaa cgtgaatagc atcatgtaaa acaatccctt gctatcagaa tgctccagtt |
| 7921 | tggccttaat tttgtaaacc ttggagagga gtggagcgcg gctgtgagaa gagatgtacc |
| 7981 | atgtactctt tctgtatcat taaagtgcag tttttatgct tct |

SEQ ID NO: 43 Mouse CYLD Isoform 2 Amino Acid Sequence (NP_001121643.1)

| | |
|---|---|
| 1 | mssglwsqek vtspyweeri fylllqecsv tdkqtqkllk vpkgsigqyi qdrsvghsrv |
| 61 | pstkgkknqi glkileqpha vlfvdekdvv einekftell laitnceerl slfrnrlrls |
| 121 | kglqvdvgsp vkvqlrsgee kfpgvvrfrg pllaertvsg iffgvellee grggqftdgv |
| 181 | yqgkqlfqcd edcgvfvald kleliedddn glesdfagpg dtmqvepppl einsrvslkv |
| 241 | gestesgtvi fcdvlpgkes lgyfvgvdmd npignwdgrf dgvqlcsfas vestillhin |
| 301 | diipdsvtqe rrppklafms rgvgdkgsss hnkpkvtgst sdpgsrnrse lfytlngssv |
| 361 | dsqqsksknp wyidevaedp aksltemssd fghsspppqp psmnslssen rfhslpfslt |
| 421 | kmpntngsma hsplslsvqs vmgelnstpv qespplpiss gnahglevgs laevkenppf |
| 481 | ygvirwigqp pglsdvlagl eledecagct dgtfrgtryf tcalkkalfv klkscrpdsr |
| 541 | faslqpvsng iercnslafg gylsevveen tppkmekegl eimigkkkgi qghynscyld |
| 601 | stlfclfafs saldtvllrp kekndieyys etqellrtei vnplriygyv catkimklrk |
| 661 | ilekveaasg ftseekdpee flnilfhdil rvepllkirs agqkvqdcnf yqifmeknek |
| 721 | vgvptiqqll ewsfinsnlk faeapsclii qmprfgkdfk lfkkifpsle lnitdlledt |
| 781 | prqcricggl amyecrecyd dpdisagkik qfcktcstqv hlhprrlnhs yhpvslpkdl |
| 841 | pdwdwrhgci pcqkmelfav lcietshyva fvkygkddsa wlffdsmadr dggqngfnip |
| 901 | qvtpcpevge ylkmsledlh sldsrriqgc arrllcdaym cmyqsptmsl yk |

SEQ ID NO: 44 Mouse CYLD transcript variant 2 cDNA sequence
(NM_001128171,2; CDS: 357 . . . 3215)

| | |
|---|---|
| 1 | agttcggagg cggggcaggt gggggcgggc ccaggtagca ggttcggctg cgcgggggcc |
| 61 | cgcgcgcctg agcagtgagc tcctatgtca tggattctga actggcttat aatatctggc |
| 121 | atggattcct tttagcagag caggcctcaa atccgataga gcatggttgc ttgatcccat |
| 181 | tgcagccctg ccactagtgg ctcctggcac tttgaattgc tgtcttttta caacatggat |
| 241 | gccaggttgc taaaatcttg ctttgggact tacagcgagt tcattttttt ggattttatg |
| 301 | acagatttac tagtggctcc tttttgccag tcaatgttct gaaagttact gtcacaatga |
| 361 | gttcaggcct gtggagccaa gagaaagtta cttcacccta ctgggaagaa cggatttttt |
| 421 | atctgcttct tcaagaatgc agtgtaacag acaaacaaac tcagaagctg ctgaaagtac |
| 481 | ccaaagggag tataggacag tacatccaag accgttctgt ggggcattca agagttcctt |
| 541 | ccacaaaagg caagaaaaat cagattggat taaaaatctt ggagcaaccg catgcagttc |
| 601 | tgtttgttga tgaaaaggat gttgtagaaa taaatgaaaa attcacagag ttactgttgg |
| 661 | caattaccaa ctgtgaggag aggctcagcc tatttagaaa cagactccga ctaagtaaag |
| 721 | gcctccaggt agacgtgggc agtcctgtga aagtacagct gcgatctggg gaagagaaat |
| 781 | ttccaggagt tgtacgcttc agaggacctt tattagcgga gaggacggtg tcggggattt |
| 841 | tctttggagt agaattattg gaagaaggtc gtggtcaagg tttcacggat ggggtatatc |
| 901 | aagggaagca gcttttccag tgtgatgagg actgtggcgt gtttgttgca ttggacaaac |
| 961 | tagaacttat agaagatgat gacaatggat tggaaagtga ttttgcaggc ccaggagata |
| 1021 | caatgcaggt tgaacctccc cctttggaaa taaactccag agtttctttg aaggttggag |
| 1081 | aaagtacaga atctggaaca gtaatattct gtgatgtttt accaggaaaa gagagtctag |
| 1141 | gatattttgt tggtgtggac atggataacc ctattggcaa ctgggatgga aggtttgatg |
| 1201 | gagtacagct ctgtagtttt gcaagtgttg aaagtacaat tctcctgcac atcaatgaca |
| 1261 | tcatcccaga tagcgtgaca caggaaagga ggcctcccaa acttgccttt atgtcaagag |
| 1321 | gtgtaggtga caaaggttca tctagtcata ataaaccaaa ggttacagga tctacctcag |
| 1381 | accctggaag tagaaacaga tctgaattat tttatacctt aaatgggtca tctgttgact |
| 1441 | cacaacaatc caagtccaaa aatccatggt acattgatga agttgcagaa gaccctgcaa |
| 1501 | agtcacttac agagatgtct tcggacttcg gacattcatc tcctccaccg cagcctcctt |
| 1561 | ccatgaactc cttgtctagc gagaacagat tccactcctt acccttcagc ctgacaaaga |
| 1621 | tgcccaatac taatggcagc atggctcata gtccactctc tctgtcagtg cagtctgtga |
| 1681 | tgggggagct gaacagcaca cctgtccagg agagtccacc cttgcccatc tcttctggga |
| 1741 | atgcacacgg gctagaggtg ggctcactgg ctgaagtaaa agagaacccc ccgttctatg |
| 1801 | gggttatccg ttggattggc cagccaccag ggctcagtga cgtgctagct ggactggaac |
| 1861 | tggaagatga atgcgcaggc tgtacagatg gaactttcag gggcacgcgg tatttcacgt |
| 1921 | gtgccctgaa gaaggcactg tttgtgaaac tgaagagctg cagaccggac tctaggtttg |
| 1981 | catccttgca gcctgtttcc aatcaaattg aaaggtgtaa ctctttagca tttgggggct |
| 2041 | atttaagtga agtagtagaa gaaaatactc cacctaaaat ggaaaaggaa ggtttagaga |
| 2101 | taatgattgg aaagaagaaa ggcatccagg gccattacaa ttcttgttac ttagactcaa |
| 2161 | ctttattctg cttatttgct tttagttctg ccctggacac tgtgttactt agacccaaag |
| 2221 | agaagaatga tatagagtat tacagtgaga ctcaggagct actgaggaca gagatagtca |
| 2281 | atcctctgag aatatatgga tatgtgtgtg ccacaaagat tatgaaactg aggaaaatac |
| 2341 | ttgaaaaagt tgaggctgca tcaggtttta cctctgaaga aaaagatcct gaagaattct |
| 2401 | taaatatcct gtttcatgat attttaaggg ttgaaccatt gttaaaaata agatcagcag |
| 2461 | gtcaaaaagt tcaagactgt aacttctatc aaatttttat ggaaaaaaat gagaaagttg |
| 2521 | gagtacccac aattcagcag ttattagaat ggtcttttat caacagcaac ctgaaatttg |
| 2581 | cagaggcacc atcatgcttg attatccaga tgcctcggtt tggaaaagac tttaaactat |
| 2641 | ttaaaaaaat ttttccttcc ctggaattaa atataacaga tttacttgaa gacactccca |
| 2701 | ggcagtgccg catctgtgga ggacttgcga tgtacgagtg cagggagtgc tatgacgatc |
| 2761 | cggacatctc agctgggaag atcaagcagt tctgtaagac ctgcagcact caggttcacc |
| 2821 | ttcatcccag gaggttgaat cattcttatc atccagtatc acttcccaaa gacttgcctg |
| 2881 | actgggactg gagacacggc tgcatcccct gtcagaagat ggagttattt gctgttctct |
| 2941 | gcatagaaac gagccactat gttgcttttg tgaagtatgg gaaggatgac tctgcctggc |
| 3001 | ttttctttga cagcatggcg gatcgagatg gtggtcagaa tggcttcaac attccacaag |
| 3061 | tgacgccctg cccagaagtg ggagagtact tgaagatgtc tctggaggac ctgcactctt |
| 3121 | tggattccag aaggattcaa ggctgtgcgc gcagacttct ttgcgatgca tacatgtgca |
| 3181 | tgtaccagag tccaaccatg agcctgtaca aataaccagg ttatccgcag tgggcagaga |
| 3241 | cagcacaaac cttagtgctg tgtcttcctc cagcttgtgg ttctgtccgg cacccattgc |
| 3301 | cggcaatggg tgtctctttt gatgatggcc cttcagcaaa ggattcctgt gtttccaaac |
| 3361 | aaattgcttt tgtgttcctg aagtatttaa taagaagcat tttgcactct aggaagtatg |
| 3421 | tttgtgttga ttttttaaga agtctaaatg gatttattaa tatctgacac tttaagttaa |
| 3481 | ctgcattgat gatacgatat ttttggaagc atacaatttt aattgtgacg tctaaaacct |
| 3541 | cttttagtcc attgaggatg taaataaata tttcttctta tggaccaggg atatgaaatc |
| 3601 | attttccttt tgtagctaat gattgccttg agaaagacat aatatgattt tattaagagt |
| 3661 | ctgcctttac tccaattgtt gaaggtgtgc tgagtttaat tgttaatttt cctatatagt |
| 3721 | gctgatttcc tgcatgtctg tactttgctg agtttctgtg ttcctgcagc agtggtcaga |
| 3781 | acatgctgaa attcctttca tggtgttatt ttttatttgt tctagttttg agataaatga |
| 3841 | gtgactctgt cataaaatgt ccatttttga tttgttcttg aagaattagg gtacaaaaca |
| 3901 | agtatcgtag tcatagtaac aactgttact gaacaggtct tctgtagggg ctcctaagta |
| 3961 | tgagacaaca tgcagtggtt tctgaaatga ctgagcgata agtaaattat atattctttt |
| 4021 | tttaaaagat ttattttatt tatatgagta cactgtagct gtcttcagac acaccagaag |
| 4081 | agggcatcag atcttattac agatggttgt gagccaccat gtggttgctg ggaattgaac |
| 4141 | tcaggacctt tggaagggca gtcagtgctc ttaactgctg agctatctct ctagccccaa |
| 4201 | attatatatt cttatacaga cccacagtcc agagaaagtg aagctggctc agagttagca |
| 4261 | ccaacatttc cctgaaaaaa tgtgttaggt gtttaaaaat ctcatcgtaa ataaattatt |
| 4321 | ttatttttag tttaagtgaa tttctcttaa tttaaataag actaatattt ttgtttgact |
| 4381 | gaagaccctt gtactctctg gtgtcagttt ggtgtcactt gaaacaatga caaccacgtc |
| 4441 | agcatgttac tgcaatgtca gtgatggttt caaaggagaa aatggatgtc atcttaggac |
| 4501 | tgcttgtgtg tgtatagacc gtggtggatg ctgtagagtg cttgttgtct gagtgttgtt |
| 4561 | ttgtggtatg gctgtctctc tgaataatgt caaccttaca gaaagagaag gtgttatatg |
| 4621 | tgtaacatat ggctttataa aagatttaat gtcaaaagag tctataaata ttgctgttat |
| 4681 | ttttaaaggc actttactcc ttggttttat tattccattt tgctggcgtg tattagcttt |
| 4741 | atataaatca caacacacaa aactcgtcca tcttgtaatc tttgtacttt tgcaatggaa |
| 4801 | aagtgtgtat actgtgcaca gacttttata tagctgagtg cctagactgg gtggagaggc |
| 4861 | catgagtaga tagcagtaaa gtcctccagc tataggctag ccttgtcttc atcatcctgg |
| 4921 | gacaggtgag aacggtgagc acaggtgagt ctgagcttgc acactgacca gaattcagtc |
| 4981 | accctgtaat aagtatgtat ttaatgagtc tgttagtgtg gtagtgtgat tttgaaaggt |
| 5041 | ttaactaagg aatggtgagg tctttgttta taggcaagcc tggcttaaca gtgaagggga |
| 5101 | gggggctgag ggaaagcttt tttttttttt aatttcagaa atggaagcaa aatccatgtg |
| 5161 | caaagagact agctacttgg ctttcagagc agaggactca aagcttgttt gcagtggttc |
| 5221 | tgaagttggg gtggcacttt gtgagtgagg aggaccatgt ccacaggcaa tccctaagca |
| 5281 | agatcgtctc gaaattgcac tgtgttcttt agcagtagtg gctgggtagg ggcacacagc |
| 5341 | ggaacaccca cttgcagtgg ctggggaggc ctgagaagca caggccagcg ggagctctct |
| 5401 | cccatgcctt cttgtccctc tggacagccc ctcctcattc cttgtcccag cagagcctgg |
| 5461 | tttgcccctg gggcaatagg gttggactgt gtggtgcaga tagccctcgt gcctgtggat |
| 5521 | tactttaatt ttgtaatctt aggttttctg tacggttttc ccattgcctt ttaaaatatt |
| 5581 | ttgagtgtcc ttctccttat caagatggta gtgtagtagg attgttttac tcacaacatg |
| 5641 | gtttaggagc catagctaca aataggaaat ataagaattc tatttctcta ctgtgaggca |
| 5701 | aatctgtata tatgtatgga gagtcaggag gatgaacaag ggagaggaaa ggggcccagg |
| 5761 | cacattttta atccaaaagc tacaaagtgt cgagctcctc agggccagct tcacagagag |
| 5821 | tccagctcag ttcacaaagt attttctaag ttggggaaat gcattgagaa acagagcgag |
| 5881 | atatctgtgt agtgcagctg tgggtggttg ctatgcattt tatcctcaga gtagcctggg |
| 5941 | gtttttattt tatgtttgcc aagtgcttag gtaaatggcc gggctcctct tacctctgac |
| 6001 | ttctggagcg tgtccattct ctggttgact gaatgtggca ttttcataaa tctaggagca |
| 6061 | gcagccctcc caactgcaat ttagtaagca ggtagataag cttgcatctg tttgcatctt |
| 6121 | cctgcactga gttgggagtg caaaaccccg tacttggcct ggtgagtaac tgccaacaag |
| 6181 | tatttatggg tcatttgcta ggtggctagc atacggcacg gggctaatga gacctggggc |
| 6241 | ctgctctcat atagcttact gctcagtttc tataccagag catccgttgg tgcttcttag |
| 6301 | cctaggcatg ggtgacaggc atgtctgggg accactttgg gagacaacct agtgtaacct |
| 6361 | tattgcaaag gactaagaga caaaaatcct gggttctttc tccatcactt gctctgtagc |
| 6421 | tgaatgaaca ccacctcgat ttcttgagtt tcagtgtctt gttctgtctg atgaaagggt |
| 6481 | tggatatgta gttggataag cggtacaaat tgttagggtt ccaggccatg cctgatacat |
| 6541 | ctgagttttt gctggaggta ggaaatttca actcagaagc tcatccacag aaaacagtga |
| 6601 | ccagcagcag gtatggagcc ataaggctgg aaggcttcaa agaccccttc ttctgttact |
| 6661 | gttatccacg gcagttattc tgtgtaggtg tgaaggagtg gctactgact ctgtgtggat |
| 6721 | cagcgtttac atatgacctg atgggagtgc atctggtatg tgttcctccc ccctgtattg |
| 6781 | ctggagagat tggcaatcgt aggtatacac ggaagtgtcg ggatgttcag tggcgcgcac |
| 6841 | ttggatgttt cttttcactt tatttctata gaaacaggtt ccaggagcca gagaagtagg |
| 6901 | ggaaaaaatc cctgagctca tctctctcta ctacttaata atgcaaacat ttaaattctt |
| 6961 | gggaagattt tcccgcccca ctccctaccc ccagtcacag gtcagaatag gatttccttc |
| 7021 | ttagagaagg taggtcatgg ggcctgcgat taaaaaaaca aaaacataaa acaaacaaac |
| 7081 | aaacaaggtg gctttccttg gtctttgtga ttccagctct tcaccagtga aggaattctt |
| 7141 | tagttccata gtgcagcctg ggtgggtgga gaagaggctg ttatagtggg agccagtgag |
| 7201 | caaagagggt tggaggaggc agaggacagg gctcagggaa ggcaagcctg tggctctggg |
| 7261 | cattggccct cttgtcaaag gtgtcagagc atggcttcct gtgaggggct ttcccttgct |
| 7321 | cctgtgtgag aatctgtgcg ttgtcttact ggatggggga cctggatgga gatgttaggg |
| 7381 | gagatagctt agtcttgttc taaactctag acttgctaaa aagggttctc ttgagtccaa |
| 7441 | atgcagtgcc agagggtgag aagcagaatt ggccagtagt gaacagtggt attgtgggtg |
| 7501 | ataagccctt tctggtggcc ttaaatagca gtgctagtcc ttgagtgcca cagggtcccc |
| 7561 | tacctccgtc ctggcagtgc tgatgggttt gtttcagcat attttggttc tcattctcat |
| 7621 | ttagggaaaa gggctgttga tgaagggatt atcctcccag gcaccagggg cctcagcaca |
| 7681 | gttccctgtg cttttaaaca ctgttgactt tcattagtta ttctaaaggg atttatattg |
| 7741 | gagatacaac tccctttggt ttcatattat gaaatagctt tgtactgtgg aatgctaact |
| 7801 | ctggaaagtc tccctcatac cagaaccaaa cattcatttc ttttatatat atatatatat |
| 7861 | tatctccctg gtactctcca gggaggtggt tattatcaag ctgacccaaa ggagtgcagt |
| 7921 | atgtgttcta gttccttttc tggtaccttg ttcaacattc ttttttcctg aagaacatcc |
| 7981 | ttttgtttat gaaaacgtga atagcatcat gtaaaacaat cccttgctat cagaatgctc |
| 8041 | cagtttggcc ttaattttgt aaaccttgga gaggagtgga gcgcggctgt gagaagagat |
| 8101 | gtaccatgta ctctttctgt atcattaaag tgcagttttt atgcttct |

SEQ ID NO: 45 Mouse CYLD transcript variant 3 cDNA sequence (NM_173369.3;
CDS: 223 . . . 3081)

| 1 | agttcggagg cggggcaggt gggggcgggc ccaggtagca ggttcggctg cgcgggggcc |
|---|---|
| 61 | cgcgcgcctg aggcactttg aattgctgtc tttttacaac atggatgcca ggttgctaaa |
| 121 | atcttgcttt gggacttaca gcgagttcat ttttttggat tttatgacag atttactagt |
| 181 | ggctcctttt tgccagtcaa tgttctgaaa gttactgtca caatgagttc aggcctgtgg |
| 241 | agccaagaga aagttacttc accctactgg gaagaacgga ttttttatct gcttcttcaa |
| 301 | gaatgcagtg taacagacaa acaaactcag aagctgctga aagtacccaa agggagtata |
| 361 | ggacagtaca tccaagaccg ttctgtgggg cattcaagag ttccttccac aaaaggcaag |
| 421 | aaaaatcaga ttggattaaa aatcttggag caaccgcatg cagttctgtt tgttgatgaa |
| 481 | aaggatgttg tagaaataaa tgaaaaattc acagagttac tgttggcaat taccaactgt |
| 541 | gaggagaggc tcagcctatt tagaaacaga ctccgactaa gtaaaggcct ccaggtagac |
| 601 | gtgggcagtc ctgtgaaagt acagctgcga tctggggaag agaaatttcc aggagttgta |
| 661 | cgcttcagag gacctttatt agcggagagg acggtgtcgg ggattttctt tggagtagaa |
| 721 | ttattggaag aaggtcgtgg tcaaggtttc acggatgggg tatatcaagg gaagcagctt |
| 781 | ttccagtgtg atgaggactg tggcgtgttt gttgcattgg acaaactaga acttatagaa |
| 841 | gatgatgaca atggattgga aagtgatttt gcaggcccag gagatacaat gcaggttgaa |
| 901 | cctccccctt tggaaataaa ctccagagtt tctttgaagg ttggagaaag tacagaatct |
| 961 | ggaacagtaa tattctgtga tgttttacca ggaaaagaga gtctaggata ttttgttggt |
| 1021 | gtggacatgg ataaccctat tggcaactgg gatggaaggt ttgatggagt acagctctgt |
| 1081 | agttttgcaa gtgttgaaag tacaattctc ctgcacatca atgacatcat cccagatagc |
| 1141 | gtgacacagg aaaggaggcc tcccaaactt gcctttatgt caagaggtgt aggtgacaaa |
| 1201 | ggttcatcta gtcataataa accaaaggtt acaggatcta cctcagaccc tggaagtaga |
| 1261 | aacagatctg aattatttta taccttaaat gggtcatctg ttgactcaca acaatccaag |
| 1321 | tccaaaaatc catggtacat tgatgaagtt gcagaagacc ctgcaaagtc acttacagag |
| 1381 | atgtcttcgg acttcggaca ttcatctcct ccaccgcagc ctccttccat gaactccttg |
| 1441 | tctagcgaga acagattcca ctccttaccc ttcagcctga caaagatgcc caatactaat |
| 1501 | ggcagcatgg ctcatagtcc actctctctg tcagtgcagt ctgtgatggg ggagctgaac |
| 1561 | agcacacctg tccaggagag tccacccttg cccatctctt ctgggaatgc acacgggcta |
| 1621 | gaggtgggct cactggctga agtaaaagag aaccccccgt tctatggggt tatccgttgg |
| 1681 | attggccagc caccagggct cagtgacgtg ctagctggac tggaactgga agatgaatgc |
| 1741 | gcaggctgta cagatggaac tttcaggggc acgcggtatt tcacgtgtgc cctgaagaag |
| 1801 | gcactgtttg tgaaactgaa gagctgcaga ccggactcta ggtttgcatc cttgcagcct |
| 1861 | gtttccaatc aaattgaaag gtgtaactct ttagcatttg ggggctattt aagtgaagta |
| 1921 | gtagaagaaa atactccacc taaaatggaa aaggaaggtt tagagataat gattggaaag |
| 1981 | aagaaaggca tccagggcca ttacaattct tgttacttag actcaacttt attctgctta |
| 2041 | tttgctttta gttctgccct ggacactgtg ttacttagac ccaaagagaa gaatgatata |
| 2101 | gagtattaca gtgagactca ggagctactg aggacagaga tagtcaatcc tctgagaata |
| 2161 | tatggatatg tgtgtgccac aaagattatg aaactgagga aaatacttga aaaagttgag |
| 2221 | gctgcatcag gttttacctc tgaagaaaaa gatcctgaag aattcttaaa tatcctgttt |
| 2281 | catgatattt taagggttga accattgtta aaaataagat cagcaggtca aaaagttcaa |
| 2341 | gactgtaact tctatcaaat ttttatggaa aaaaatgaga aagttggagt acccacaatt |
| 2401 | cagcagttat tagaatggtc ttttatcaac agcaacctga aatttgcaga ggcaccatca |
| 2461 | tgcttgatta tccagatgcc tcggtttgga aaagacttta aactatttaa aaaaattttt |
| 2521 | ccttccctgg aattaaatat aacagattta cttgaagaca ctcccaggca gtgccgcatc |
| 2581 | tgtggaggac ttgcgatgta cgagtgcagg gagtgctatg acgatccgga catctcagct |
| 2641 | gggaagatca agcagttctg taagacctgc agcactcagg ttcaccttca tcccaggagg |
| 2701 | ttgaatcatt cttatcatcc agtatcactt cccaaagact tgcctgactg ggactggaga |
| 2761 | cacggctgca tcccctgtca gaagatggag ttatttgctg ttctctgcat agaaacgagc |
| 2821 | cactatgttg cttttgtgaa gtatgggaag gatgactctg cctggctttt ctttgacagc |
| 2881 | atggcggatc gagatggtgg tcagaatggc ttcaacattc cacaagtgac gccctgccca |
| 2941 | gaagtgggag agtacttgaa gatgtctctg gaggacctgc actctttgga ttccagaagg |
| 3001 | attcaaggct gtgcgcgcag acttctttgc gatgcataca tgtgcatgta ccagagtcca |
| 3061 | accatgagcc tgtacaaata accaggttat ccgcagtggg cagagacagc acaaacctta |
| 3121 | gtgctgtgtc ttcctccagc ttgtggttct gtccggcacc cattgccggc aatgggtgtc |
| 3181 | tcttttgatg atggcccttc agcaaaggat tcctgtgttt ccaaacaaat tgcttttgtg |
| 3241 | ttcctgaagt atttaataag aagcattttg cactctagga agtatgtttg tgttgatttt |
| 3301 | ttaagaagtc taaatggatt tattaatatc tgacacttta agttaactgc attgatgata |
| 3361 | cgatattttt ggaagcatac aattttaatt gtgacgtcta aaacctcttt tagtccattg |
| 3421 | aggatgtaaa taaatatttc ttcttatgga ccagggatat gaaatcattt tccttttgta |
| 3481 | gctaatgatt gccttgagaa agacataata tgattttatt aagagtctgc ctttactcca |
| 3541 | attgttgaag gtgtgctgag tttaattgtt aattttccta tatagtgctg atttcctgca |
| 3601 | tgtctgtact ttgctgagtt tctgtgttcc tgcagcagtg gtcagaacat gctgaaattc |
| 3661 | ctttcatggt gttatttttt atttgttcta gttttgagat aaatgagtga ctctgtcata |
| 3721 | aaatgtccat ttttgatttg ttcttgaaga attagggtac aaaacaagta tcgtagtcat |
| 3781 | agtaacaact gttactgaac aggtcttctg taggggctcc taagtatgag acaacatgca |
| 3841 | gtggtttctg aaatgactga gcgataagta aattatatat tcttttttta aaagatttat |
| 3901 | tttatttata tgagtacact gtagctgtct tcagacacac cagaagaggg catcagatct |
| 3961 | tattacagat ggttgtgagc caccatgtgg ttgctgggaa ttgaactcag gacctttgga |
| 4021 | agggcagtca gtgctcttaa ctgctgagct atctctctag ccccaaatta tatattctta |
| 4081 | tacagaccca cagtccagag aaagtgaagc tggctcagag ttagcaccaa catttccctg |
| 4141 | aaaaaatgtg ttaggtgttt aaaaatctca tcgtaaataa attattttat ttttagttta |
| 4201 | agtgaatttc tcttaattta aataagacta atatttttgt ttgactgaag acccttgtac |
| 4261 | tctctggtgt cagtttggtg tcacttgaaa caatgacaac cacgtcagca tgttactgca |
| 4321 | atgtcagtga tggtttcaaa ggagaaaatg gatgtcatct taggactgct tgtgtgtgta |
| 4381 | tagaccgtgg tggatgctgt agagtgcttg ttgtctgagt gttgttttgt ggtatggctg |
| 4441 | tctctctgaa taatgtcaac cttacagaaa gagaaggtgt tatatgtgta acatatggct |
| 4501 | ttataaaaga tttaatgtca aaagagtcta taaatattgc tgttattttt aaaggcactt |
| 4561 | tactccttgg ttttattatt ccattttgct ggcgtgtatt agctttatat aaatcacaac |
| 4621 | acacaaaact cgtccatctt gtaatctttg tacttttgca atggaaaagt gtgtatactg |
| 4681 | tgcacagact tttatatagc tgagtgccta gactgggtgg agaggccatg agtagatagc |
| 4741 | agtaaagtcc tccagctata ggctagcctt gtcttcatca tcctgggaca ggtgagaacg |
| 4801 | gtgagcacag gtgagtctga gcttgcacac tgaccagaat tcagtcaccc tgtaataagt |
| 4861 | atgtatttaa tgagtctgtt agtgtggtag tgtgattttg aaaggtttaa ctaaggaatg |
| 4921 | gtgaggtctt tgtttatagg caagcctggc ttaacagtga aggggagggg gctgagggaa |
| 4981 | agcttttttt ttttttaatt tcagaaatgg aagcaaaatc catgtgcaaa gagactagct |
| 5041 | acttggcttt cagagcagag gactcaaagc ttgtttgcag tggttctgaa gttggggtgg |
| 5101 | cactttgtga gtgaggagga ccatgtccac aggcaatccc taagcaagat cgtctcgaaa |
| 5161 | ttgcactgtg ttctttagca gtagtggctg ggtaggggca cacagcggaa cacccacttg |
| 5221 | cagtggctgg ggaggcctga gaagcacagg ccagcgggag ctctctccca tgccttcttg |
| 5281 | tccctctgga cagcccctcc tcattccttg tcccagcaga gcctggtttg cccctggggc |
| 5341 | aatagggttg gactgtgtgg tgcagatagc cctcgtgcct gtggattact ttaattttgt |
| 5401 | aatcttaggt tttctgtacg gttttcccat tgccttttaa aatattttga gtgtccttct |
| 5461 | ccttatcaag atggtagtgt agtaggattg ttttactcac aacatggttt aggagccata |
| 5521 | gctacaaata ggaaatataa gaattctatt tctctactgt gaggcaaatc tgtatatatg |
| 5581 | tatggagagt caggaggatg aacaagggag aggaaagggg cccaggcaca tttttaatcc |
| 5641 | aaaagctaca aagtgtcgag ctcctcaggg ccagcttcac agagagtcca gctcagttca |
| 5701 | caaagtattt tctaagttgg ggaaatgcat tgagaaacag agcgagatat ctgtgtagtg |
| 5761 | cagctgtggg tggttgctat gcattttatc ctcagagtag cctggggttt ttattttatg |
| 5821 | tttgccaagt gcttaggtaa atggccgggc tcctcttacc tctgacttct ggagcgtgtc |
| 5881 | cattctctgg ttgactgaat gtggcatttt cataaatcta ggagcagcag ccctcccaac |
| 5941 | tgcaatttag taagcaggta gataagcttg catctgtttg catcttcctg cactgagttg |
| 6001 | ggagtgcaaa accccgtact tggcctggtg agtaactgcc aacaagtatt tatgggtcat |
| 6061 | ttgctaggtg gctagcatac ggcacggggc taatgagacc tggggcctgc tctcatatag |
| 6121 | cttactgctc agtttctata ccagagcatc cgttggtgct tcttagccta ggcatgggtg |
| 6181 | acaggcatgt ctggggacca ctttgggaga caacctagtg taaccttatt gcaaaggact |
| 6241 | aagagacaaa aatcctgggt tctttctcca tcacttgctc tgtagctgaa tgaacaccac |
| 6301 | ctcgatttct tgagtttcag tgtcttgttc tgtctgatga aagggttgga tatgtagttg |
| 6361 | gataagcggt acaaattgtt agggttccag gccatgcctg atacatctga gtttttgctg |
| 6421 | gaggtaggaa atttcaactc agaagctcat ccacagaaaa cagtgaccag cagcaggtat |
| 6481 | ggagccataa ggctggaagg cttcaaagac cccttcttct gttactgtta tccacggcag |
| 6541 | ttattctgtg taggtgtgaa ggagtggcta ctgactctgt gtggatcagc gtttacatat |
| 6601 | gacctgatgg gagtgcatct ggtatgtgtt cctcccccct gtattgctgg agagattggc |
| 6661 | aatcgtaggt atacacggaa gtgtcgggat gttcagtggc gcgcacttgg atgtttcttt |
| 6721 | tcactttatt tctatagaaa caggttccag gagccagaga agtaggggaa aaaatccctg |
| 6781 | agctcatctc tctctactac ttaataatgc aaacatttaa attcttggga agattttccc |
| 6841 | gccccactcc ctacccccag tcacaggtca gaataggatt tccttcttag agaaggtagg |
| 6901 | tcatggggcc tgcgattaaa aaaacaaaaa cataaaacaa acaaacaaac aaggtggctt |
| 6961 | tccttggtct ttgtgattcc agctcttcac cagtgaagga attctttagt tccatagtgc |
| 7021 | agcctgggtg ggtggagaag aggctgttat agtgggagcc agtgagcaaa gagggttgga |
| 7081 | ggaggcagag gacagggctc agggaaggca agcctgtggc tctgggcatt ggccctcttg |
| 7141 | tcaaaggtgt cagagcatgg cttcctgtga ggggctttcc cttgctcctg tgtgagaatc |
| 7201 | tgtgcgttgt cttactggat gggggacctg gatggagatg ttaggggaga tagcttagtc |
| 7261 | ttgttctaaa ctctagactt gctaaaaagg gttctcttga gtccaaatgc agtgccagag |
| 7321 | ggtgagaagc agaattggcc agtagtgaac agtggtattg tgggtgataa gccctttctg |
| 7381 | gtggccttaa atagcagtgc tagtccttga gtgccacagg gtcccctacc tccgtcctgg |
| 7441 | cagtgctgat gggtttgttt cagcatattt tggttctcat tctcatttag ggaaaagggc |
| 7501 | tgttgatgaa gggattatcc tcccaggcac caggggcctc agcacagttc cctgtgcttt |
| 7561 | taaacactgt tgactttcat tagttattct aaagggattt atattggaga tacaactccc |
| 7621 | tttggtttca tattatgaaa tagctttgta ctgtggaatg ctaactctgg aaagtctccc |
| 7681 | tcataccaga accaaacatt catttctttt atatatatat atatattatc tccctggtac |
| 7741 | tctccaggga ggtggttatt atcaagctga cccaaaggag tgcagtatgt gttctagttc |
| 7801 | cttttctggt accttgttca acattctttt ttcctgaaga acatcctttt gtttatgaaa |
| 7861 | acgtgaatag catcatgtaa aacaatccct tgctatcaga atgctccagt ttggccttaa |
| 7921 | ttttgtaaac cttggagagg agtggagcgc ggctgtgaga agagatgtac catgtactct |
| 7981 | ttctgtatca ttaaagtgca gtttttatgc ttct |

SEQ ID NO: 46 Mouse CYLD Isoform 3 Amino Acid Sequence (NP_001263208.1)

| | |
|---|---|
| 1 | mssglwsqek vtspyweeri fylllqecsv tdkqtqkllk vpkgsigqyi qdrsvghsrv |
| 61 | pstkgkknqi glkileqpha vlfvdekdvv einekftell laitnceerl slfrnrlrls |
| 121 | kglqvdvgsp vkvqlrsgee kfpgvvrfrg pllaertvsg iffgvellee grgqgftdgv |
| 181 | yqgkqlfqcd edcgvfvald kleliedddn glesdfagpg dtmqvepppl einsrvslkv |
| 241 | gestesgtvi fcdvlpgkes lgyfvgvdmd npignwdgrf dgvqlcsfas vestillhin |
| 301 | diipdsvtqe rrppklafms rgvgdkgsss hnkpkvtgst sdpgsrnrse lfytlngssv |
| 361 | dsqqsksknp wyideafggy lsevveentp pkmekeglei migkkkgiqg hynscyldst |
| 421 | lfclfafssa ldtvllrpke kndieyyset qellrteivn plriygyvca tkimklrkil |
| 481 | ekveaasgft seekdpeefl nilfhdilrv epllkirsag qkvqdcnfyq ifmeknekvg |
| 541 | vptiqqllew sfinsnlkfa eapscliiqm prfgkdfklf kkifpsleln itdlledtpr |
| 601 | qcricgglam yecrecyddp disagkikqf cktcstqvhl hprrlnhsyh pvslpkdlpd |
| 661 | wdwrhgcipc qkmelfavlc ietshyvafv kygkddsawl ffdsmadrdg gqngfnipqv |
| 721 | tpcpevgeyl kmsledlhsl dsrriqgcar rllcdaymcm yqsptmslyk |

SEQ ID NO: 47 Mouse CYLD transcript variant 4 cDNA sequence
(NM_001276279,1; CDS: 354 . . . 2666)

| | |
|---|---|
| 1 | agttcggagg cggggcaggt gggggcgggc ccaggtagca ggttcggctg cgcgggggcc |
| 61 | cgcgcgcctg agtgagctcc tatgtcatgg attctgaact ggcttataat atctggcatg |
| 121 | gattcctttt agcagagcag gcctcaaatc cgatagagca tggttgcttg atcccattgc |
| 181 | agccctgcca ctagtggctc ctggcacttt gaattgctgt ctttttacaa catggatgcc |
| 241 | aggttgctaa aatcttgctt tgggacttac agcgagttca tttttttgga ttttatgaca |
| 301 | gatttactag tggctccttt ttgccagtca atgttctgaa agttactgtc acaatgagtt |
| 361 | caggcctgtg gagccaagag aaagttactt caccctactg ggaagaacgg attttttatc |
| 421 | tgcttcttca agaatgcagt gtaacagaca aacaaactca gaagctgctg aaagtaccca |
| 481 | aagggagtat aggacagtac atccaagacc gttctgtggg gcattcaaga gttccttcca |
| 541 | caaaaggcaa gaaaaatcag attggattaa aaatcttgga gcaaccgcat gcagttctgt |
| 601 | ttgttgatga aaaggatgtt gtagaaataa atgaaaaatt cacagagtta ctgttggcaa |
| 661 | ttaccaactg tgaggagagg ctcagcctat ttagaaacag actccgacta agtaaaggcc |
| 721 | tccaggtaga cgtgggcagt cctgtgaaag tacagctgcg atctggggaa gagaaatttc |
| 781 | caggagttgt acgcttcaga ggacctttat tagcggagag gacggtgtcg gggattttct |
| 841 | ttggagtaga attattggaa gaaggtcgtg gtcaaggttt cacggatggg gtatatcaag |
| 901 | ggaagcagct tttccagtgt gatgaggact gtggcgtgtt tgttgcattg gacaaactag |
| 961 | aacttataga agatgatgac aatggattgg aaagtgattt tgcaggccca ggagatacaa |
| 1021 | tgcaggttga acctccccct ttggaaataa actccagagt ttctttgaag gttggagaaa |
| 1081 | gtacagaatc tggaacagta atattctgtg atgttttacc aggaaaagag agtctaggat |
| 1141 | attttgttgg tgtggacatg gataacccta ttggcaactg ggatggaagg tttgatggag |
| 1201 | tacagctctg tagttttgca agtgttgaaa gtacaattct cctgcacatc aatgacatca |
| 1261 | tcccagatag cgtgacacag gaaaggaggc ctcccaaact tgcctttatg tcaagaggtg |
| 1321 | taggtgacaa aggttcatct agtcataata aaccaaaggt tacaggatct acctcagacc |
| 1381 | ctggaagtag aaacagatct gaattatttt ataccttaaa tgggtcatct gttgactcac |
| 1441 | aacaatccaa gtccaaaaat ccatggtaca ttgatgaagc atttgggggc tatttaagtg |
| 1501 | aagtagtaga agaaaatact ccacctaaaa tggaaaagga aggtttagag ataatgattg |
| 1561 | gaaagaagaa aggcatccag ggccattaca attcttgtta cttagactca actttattct |
| 1621 | gcttatttgc ttttagttct gccctggaca ctgtgttact tagacccaaa gagaagaatg |
| 1681 | atatagagta ttacagtgag actcaggagc tactgaggac agagatagtc aatcctctga |
| 1741 | gaatatatgg atatgtgtgt gccacaaaga ttatgaaact gaggaaaata cttgaaaaag |
| 1801 | ttgaggctgc atcaggtttt acctctgaag aaaaagatcc tgaagaattc ttaaatatcc |
| 1861 | tgtttcatga tattttaagg gttgaaccat tgttaaaaat aagatcagca ggtcaaaaag |
| 1921 | ttcaagactg taacttctat caaattttta tggaaaaaaa tgagaaagtt ggagtaccca |
| 1981 | caattcagca gttattagaa tggtctttta tcaacagcaa cctgaaattt gcagaggcac |
| 2041 | catcatgctt gattatccag atgcctcggt ttggaaaaga ctttaaacta tttaaaaaaa |
| 2101 | tttttccttc cctggaatta aatataacag atttacttga agacactccc aggcagtgcc |
| 2161 | gcatctgtgg aggacttgcg atgtacgagt gcagggagtg ctatgacgat ccggacatct |
| 2221 | cagctgggaa gatcaagcag ttctgtaaga cctgcagcac tcaggttcac cttcatccca |
| 2281 | ggaggttgaa tcattcttat catccagtat cacttcccaa agacttgcct gactgggact |
| 2341 | ggagacacgg ctgcatcccc tgtcagaaga tggagttatt tgctgttctc tgcatagaaa |
| 2401 | cgagccacta tgttgctttt gtgaagtatg ggaaggatga ctctgcctgg cttttctttg |
| 2461 | acagcatggc ggatcgagat ggtggtcaga atggcttcaa cattccacaa gtgacgccct |
| 2521 | gcccagaagt gggagagtac ttgaagatgt ctctggagga cctgcactct ttggattcca |
| 2581 | gaaggattca aggctgtgcg cgcagacttc tttgcgatgc atacatgtgc atgtaccaga |
| 2641 | gtccaaccat gagcctgtac aaataaccag gttatccgca gtgggcagag acagcacaaa |
| 2701 | ccttagtgct gtgtcttcct ccagcttgtg gttctgtccg gcacccattg ccggcaatgg |
| 2761 | gtgtctcttt tgatgatggc ccttcagcaa aggattcctg tgtttccaaa caaattgctt |
| 2821 | ttgtgttcct gaagtattta ataagaagca ttttgcactc taggaagtat gtttgtgttg |
| 2881 | attttttaag aagtctaaat ggatttatta atatctgaca ctttaagtta actgcattga |
| 2941 | tgatacgata tttttggaag catacaattt taattgtgac gtctaaaacc tcttttagtc |
| 3001 | cattgaggat gtaaataaat atttcttctt atggaccagg gatatgaaat cattttcctt |
| 3061 | ttgtagctaa tgattgcctt gagaaagaca taatatgatt ttattaagag tctgccttta |
| 3121 | ctccaattgt tgaaggtgtg ctgagtttaa ttgttaattt tcctatatag tgctgatttc |
| 3181 | ctgcatgtct gtactttgct gagtttctgt gttcctgcag cagtggtcag aacatgctga |
| 3241 | aattcctttc atggtgttat tttttatttg ttctagtttt gagataaatg agtgactctg |
| 3301 | tcataaaatg tccatttttg atttgttctt gaagaattag ggtacaaaac aagtatcgta |
| 3361 | gtcatagtaa caactgttac tgaacaggtc ttctgtaggg gctcctaagt atgagacaac |
| 3421 | atgcagtggt ttctgaaatg actgagcgat aagtaaatta tatattcttt ttttaaaaga |
| 3481 | tttattttat ttatatgagt acactgtagc tgtcttcaga cacaccagaa gagggcatca |
| 3541 | gatcttatta cagatggttg tgagccacca tgtggttgct gggaattgaa ctcaggacct |
| 3601 | ttggaagggc agtcagtgct cttaactgct gagctatctc tctagcccca aattatatat |
| 3661 | tcttatacag acccacagtc cagagaaagt gaagctggct cagagttagc accaacattt |
| 3721 | ccctgaaaaa atgtgttagg tgtttaaaaa tctcatcgta aataaattat tttattttta |
| 3781 | gtttaagtga atttctctta atttaaataa gactaatatt tttgtttgac tgaagaccct |
| 3841 | tgtactctct ggtgtcagtt tggtgtcact tgaaacaatg acaaccacgt cagcatgtta |
| 3901 | ctgcaatgtc agtgatggtt tcaaaggaga aaatggatgt catcttagga ctgcttgtgt |
| 3961 | gtgtatagac cgtggtggat gctgtagagt gcttgttgtc tgagtgttgt tttgtggtat |
| 4021 | ggctgtctct ctgaataatg tcaaccttac agaaagagaa ggtgttatat gtgtaacata |
| 4081 | tggctttata aaagatttaa tgtcaaaaga gtctataaat attgctgtta tttttaaagg |
| 4141 | cactttactc cttggtttta ttattccatt ttgctggcgt gtattagctt tatataaatc |
| 4201 | acaacacaca aaactcgtcc atcttgtaat ctttgtactt ttgcaatgga aaagtgtgta |
| 4261 | tactgtgcac agacttttat atagctgagt gcctagactg ggtggagagg ccatgagtag |
| 4321 | atagcagtaa agtcctccag ctataggcta gccttgtctt catcatcctg ggacaggtga |
| 4381 | gaacggtgag cacaggtgag tctgagcttg cacactgacc agaattcagt caccctgtaa |
| 4441 | taagtatgta tttaatgagt ctgttagtgt ggtagtgtga ttttgaaagg tttaactaag |
| 4501 | gaatggtgag gtctttgttt ataggcaagc ctggcttaac agtgaagggg agggggctga |
| 4561 | gggaaagctt tttttttttt taatttcaga aatggaagca aaatccatgt gcaaagagac |
| 4621 | tagctacttg gctttcagag cagaggactc aaagcttgtt tgcagtggtt ctgaagttgg |
| 4681 | ggtggcactt tgtgagtgag gaggaccatg tccacaggca atccctaagc aagatcgtct |
| 4741 | cgaaattgca ctgtgttctt tagcagtagt ggctgggtag gggcacacag cggaacaccc |
| 4801 | acttgcagtg gctggggagg cctgagaagc acaggccagc gggagctctc tcccatgcct |
| 4861 | tcttgtccct ctggacagcc cctcctcatt ccttgtccca gcagagcctg gtttgcccct |
| 4921 | ggggcaatag ggttggactg tgtggtgcag atagccctcg tgcctgtgga ttactttaat |
| 4981 | tttgtaatct taggttttct gtacggtttt cccattgcct tttaaaatat tttgagtgtc |
| 5041 | cttctcctta tcaagatggt agtgtagtag gattgtttta ctcacaacat ggtttaggag |
| 5101 | ccatagctac aaataggaaa tataagaatt ctatttctct actgtgaggc aaatctgtat |
| 5161 | atatgtatgg agagtcagga ggatgaacaa gggagaggaa aggggcccag gcacattttt |
| 5221 | aatccaaaag ctacaaagtg tcgagctcct cagggccagc ttcacagaga gtccagctca |
| 5281 | gttcacaaag tattttctaa gttggggaaa tgcattgaga aacagagcga gatatctgtg |
| 5341 | tagtgcagct gtgggtggtt gctatgcatt ttatcctcag agtagcctgg ggtttttatt |
| 5401 | ttatgtttgc caagtgctta ggtaaatggc cgggctcctc ttacctctga cttctggagc |
| 5461 | gtgtccattc tctggttgac tgaatgtggc attttcataa atctaggagc agcagccctc |
| 5521 | ccaactgcaa tttagtaagc aggtagataa gcttgcatct gtttgcatct tcctgcactg |
| 5581 | agttgggagt gcaaaacccc gtacttggcc tggtgagtaa ctgccaacaa gtatttatgg |
| 5641 | gtcatttgct aggtggctag catacggcac ggggctaatg agacctgggg cctgctctca |
| 5701 | tatagcttac tgctcagttt ctataccaga gcatccgttg gtgcttctta gcctaggcat |
| 5761 | gggtgacagg catgtctggg gaccactttg ggagacaacc tagtgtaacc ttattgcaaa |
| 5821 | ggactaagag acaaaaatcc tgggttcttt ctccatcact tgctctgtag ctgaatgaac |
| 5881 | accacctcga tttcttgagt ttcagtgtct tgttctgtct gatgaaaggg ttggatatgt |
| 5941 | agttggataa gcggtacaaa ttgttagggt tccaggccat gcctgataca tctgagtttt |
| 6001 | tgctggaggt aggaaatttc aactcagaag ctcatccaca gaaaacagtg accagcagca |
| 6061 | ggtatggagc cataaggctg gaaggcttca aagacccctt cttctgttac tgttatccac |
| 6121 | ggcagttatt ctgtgtaggt gtgaaggagt ggctactgac tctgtgtgga tcagcgttta |
| 6181 | catatgacct gatgggagtg catctggtat gtgttcctcc cccctgtatt gctggagaga |
| 6241 | ttggcaatcg taggtataca cggaagtgtc gggatgttca gtggcgcgca cttggatgtt |
| 6301 | tcttttcact ttatttctat agaaacaggt tccaggagcc agagaagtag gggaaaaaat |
| 6361 | ccctgagctc atctctctct actacttaat aatgcaaaca tttaaattct tgggaagatt |
| 6421 | ttcccgcccc actccctacc cccagtcaca ggtcagaata ggatttcctt cttagagaag |
| 6481 | gtaggtcatg gggcctgcga ttaaaaaaac aaaaacataa aacaaacaaa caaacaaggt |
| 6541 | ggctttcctt ggtctttgtg attccagctc ttcaccagtg aaggaattct ttagttccat |
| 6601 | agtgcagcct gggtgggtgg agaagaggct gttatagtgg gagccagtga gcaaagaggg |
| 6661 | ttggaggagg cagaggacag ggctcaggga aggcaagcct gtggctctgg gcattggccc |
| 6721 | tcttgtcaaa ggtgtcagag catggcttcc tgtgaggggc tttcccttgc tcctgtgtga |
| 6781 | gaatctgtgc gttgtcttac tggatggggg acctggatgg agatgttagg ggagatagct |
| 6841 | tagtcttgtt ctaaactcta gacttgctaa aaagggttct cttgagtcca aatgcagtgc |
| 6901 | cagagggtga gaagcagaat tggccagtag tgaacagtgg tattgtgggt gataagccct |
| 6961 | ttctggtggc cttaaatagc agtgctagtc cttgagtgcc acagggtccc ctacctccgt |
| 7021 | cctggcagtg ctgatgggtt tgtttcagca tattttggtt ctcattctca tttagggaaa |
| 7081 | agggctgttg atgaagggat tatcctccca ggcaccaggg gcctcagcac agttccctgt |
| 7141 | gcttttaaac actgttgact ttcattagtt attctaaagg gatttatatt ggagatacaa |
| 7381 | agttcctttt tttcatatta tgaaatagct ttgtactgtg gaatgctaac tctggaaagt |
| 7261 | ctccctcata ccagaaccaa acattcattt cttttatata tatatatata ttatctccct |
| 7321 | ggtactctcc agggaggtgg ttattatcaa gctgacccaa aggagtgcag tatgtgttct |
| 7381 | agttcctttt ctggtacctt gttcaacatt cttttttcct gaagaacatc cttttgttta |
| 7441 | tgaaaacgtg aatagcatca tgtaaaacaa tcccttgcta tcagaatgct ccagtttggc |
| 7501 | cttaattttg taaaccttgg agaggagtgg agcgcggctg tgagaagaga tgtaccatgt |
| 7561 | actctttctg tatcattaaa gtgcagtttt tatgcttct |

TABLE 1B
AR
SEQ ID NO: 48 Human AR Isoform 1 Amino Acid Sequence (NP_000035.2)

| | |
|---|---|
| 1 | mevqlglgrv yprppsktyr gafqnlfqsv reviqnpgpr hpeaasaapp gasllllqqq |
| 61 | qqqqqqqqqq qqqqqqqqqq etsprqqqqq qgedgspqah rrgptgylvl deeqqpsqpq |
| 121 | salechperg cvpepgaava askglpqqlp appdeddsaa pstlsllgpt fpglsscsad |
| 181 | lkdilseast mqllqqqqqe avsegsssgr areasgapts skdnylggts tisdnakelc |
| 241 | kavsvsmglg vealehlspg eqlrgdcmya pllgvppavr ptpcaplaec kgsllddsag |
| 301 | kstedtaeys pfkggytkgl egeslgcsgs aaagssgtle lpstlslyks galdeaaayq |
| 361 | srdyynfpla lagppppppp phpharikle npldygsawa aaaaqcrygd laslhgagaa |
| 421 | gpgsgspsaa assswhtlft aeegqlygpc gggggggggg gggggggggg gggeagavap |
| 481 | ygytrppqgl agqesdftap dvwypggmvs rvpypsptcv ksemgpwmds ysgpygdmrl |
| 541 | etardhvlpi dyyfppqktc licgdeasgc hygaltcgsc kvffkraaeg kqkylcasrn |
| 601 | dctidkfrrk ncpscrlrkc yeagmtlgar klkklgnlkl qeegeasstt spteettqkl |
| 661 | tvshiegyec qpiflnvlea iepgvvcagh dnnqpdsfaa llsslnelge rqlvhvvkwa |
| 721 | kalpgfrnlh vddqmaviqy swmglmvfam gwrsftnvns rmlyfapdlv fneyrmhksr |
| 781 | mysqcvrmrh lsqefgwlqi tpqeflcmka lllfsiipvd glknqkffde lrmnyikeld |
| 841 | riiackrknp tscsrrfyql tklldsvqpi arelhqftfd llikshmvsv dfpemmaeii |
| 901 | svqvpkilsg kvkpiyfhtq |

SEQ ID NO: 49 Human AR transcript variant 1 cDNA sequence (NM_000044.6;
CDS; 1127 . . . 3889

| | |
|---|---|
| 1 | agcgccccct ccgagatccc ggggagccag cttgctggga gagcgggacg gtccggagca |
| 61 | agcccagagg cagaggaggc gacagaggga aaaagggccg agctagccgc tccagtgctg |
| 121 | tacaggagcc gaagggacgc accacgccag ccccagcccg gctccagcga cagccaacgc |
| 181 | ctcttgcagc gcggcggctt cgaagccgcc gcccggagct gccctttcct cttcggtgaa |
| 241 | gtttttaaaa gctgctaaag actcggagga agcaaggaaa gtgcctggta ggactgacgg |
| 301 | ctgcctttgt cctcctcctc tccaccccgc ctccccccac cctgccttcc ccccctcccc |
| 361 | cgtcttctct cccgcagctg cctcagtcgg ctactctcag ccaacccccc tcaccaccct |
| 421 | tctccccacc cgcccccccg cccccgtcgg cccagcgctg ccagcccgag tttgcagaga |
| 481 | ggtaactccc tttggctgcg agcgggcgag ctagctgcac attgcaaaga aggctcttag |
| 541 | gagccaggcg actggggagc ggcttcagca ctgcagccac gacccgcctg gttaggctgc |
| 601 | acgcggagag aaccctctgt tttcccccac tctctctcca cctcctcctg ccttccccac |
| 661 | cccgagtgcg gagccagaga tcaaaagatg aaaaggcagt caggtcttca gtagccaaaa |
| 721 | aacaaaacaa acaaaaacaa aaaagccgaa ataaaagaaa aagataataa ctcagttctt |
| 781 | atttgcacct acttcagtgg acactgaatt tggaaggtgg aggattttgt ttttttcttt |
| 841 | taagatctgg gcatcttttg aatctaccct tcaagtatta agagacagac tgtgagccta |
| 901 | gcagggcaga tcttgtccac cgtgtgtctt cttctgcacg agactttgag gctgtcagag |
| 961 | cgctttttgc gtggttgctc ccgcaagttt ccttctctgg agcttcccgc aggtgggcag |
| 1021 | ctagctgcag cgactaccgc atcatcacag cctgttgaac tcttctgagc aagagaaggg |
| 1081 | gaggcggggt aagggaagta ggtggaagat tcagccaagc tcaaggatgg aagtgcagtt |
| 1141 | agggctggga agggtctacc ctcggccgcc gtccaagacc taccgaggag ctttccagaa |
| 1201 | tctgttccag agcgtgcgcg aagtgatcca gaacccgggc cccaggcacc cagaggccgc |
| 1261 | gagcgcagca cctcccggcg ccagtttgct gctgctgcag cagcagcagc agcagcagca |
| 1321 | gcagcagcag cagcagcagc agcagcagca gcagcagcag cagcaagaga ctagccccag |
| 1381 | gcagcagcag cagcagcagg gtgaggatgg ttctccccaa gcccatcgta gaggccccac |
| 1441 | aggctacctg gtcctggatg aggaacagca accttcacag ccgcagtcgg ccctggagtg |
| 1501 | ccaccccgag agaggttgcg tcccagagcc tggagccgcc gtggccgcca gcaaggggct |
| 1561 | gccgcagcag ctgccagcac ctccggacga ggatgactca gctgccccat ccacgttgtc |
| 1621 | cctgctgggc cccactttcc ccggcttaag cagctgctcc gctgacctta aagacatcct |
| 1681 | gagcgaggcc agcaccatgc aactccttca gcaacagcag caggaagcag tatccgaagg |
| 1741 | cagcagcagc gggagagcga gggaggcctc gggggctccc acttcctcca aggacaatta |
| 1801 | cttagggggc acttcgacca tttctgacaa cgccaaggag ttgtgtaagg cagtgtcggt |
| 1861 | gtccatgggc ctgggtgtgg aggcgttgga gcatctgagt ccaggggaac agcttcgggg |
| 1921 | ggattgcatg tacgccccac ttttgggagt tccacccgct gtgcgtccca ctccttgtgc |
| 1981 | cccattggcc gaatgcaaag gttctctgct agacgacagc gcaggcaaga gcactgaaga |
| 2041 | tactgctgag tattcccctt tcaagggagg ttacaccaaa gggctagaag gcgagagcct |
| 2101 | aggctgctct ggcagcgctg cagcagggag ctccgggaca cttgaactgc cgtctaccct |
| 2161 | gtctctctac aagtccggag cactggacga ggcagctgcg taccagagtc gcgactacta |
| 2221 | caactttcca ctggctctgg ccggaccgcc gccccctccg ccgcctcccc atccccacgc |
| 2281 | tcgcatcaag ctggagaacc cgctggacta cggcagcgcc tgggcggctg cggcggcgca |
| 2341 | gtgccgctat ggggacctgg cgagcctgca tggcgcgggt gcagcgggac ccggttctgg |
| 2401 | gtcaccctca gccgccgctt cctcatcctg gcacactctc ttcacagccg aagaaggcca |
| 2461 | gttgtatgga ccgtgtggtg gtggtggggg tggtggcggc ggcggcggcg gcggcggcgg |
| 2521 | cggcggcggc ggcggcggcg gcggcgaggc gggagctgta gccccctacg gctacactcg |
| 2581 | gccccctcag gggctggcgg gccaggaaag cgacttcacc gcacctgatg tgtggtaccc |
| 2641 | tggcggcatg gtgagcagag tgccctatcc cagtcccact tgtgtcaaaa gcgaaatggg |
| 2701 | cccctggatg gatagctact ccggacctta cggggacatg cgtttggaga ctgccaggga |
| 2761 | ccatgttttg cccattgact attactttcc accccagaag acctgcctga tctgtggaga |
| 2821 | tgaagcttct gggtgtcact atggagctct cacatgtgga agctgcaagg tcttcttcaa |
| 2881 | aagagccgct gaagggaaac agaagtacct gtgcgccagc agaaatgatt gcactattga |
| 2941 | taaattccga aggaaaaatt gtccatcttg tcgtcttcgg aaatgttatg aagcagggat |
| 3001 | gactctggga gcccggaagc tgaagaaact tggtaatctg aaactacagg aggaaggaga |
| 3061 | ggcttccagc accaccagcc ccactgagga gacaacccag aagctgacag tgtcacacat |
| 3121 | tgaaggctat gaatgtcagc ccatctttct gaatgtcctg gaagccattg agccaggtgt |
| 3181 | agtgtgtgct ggacacgaca acaaccagcc cgactccttt gcagccttgc tctctagcct |
| 3241 | caatgaactg ggagagagac agcttgtaca cgtggtcaag tgggccaagg ccttgcctgg |
| 3301 | cttccgcaac ttacacgtgg acgaccagat ggctgtcatt cagtactcct ggatggggct |
| 3361 | catggtgttt gccatgggct ggcgatcctt caccaatgtc aactccagga tgctctactt |
| 3421 | cgcccctgat ctggttttca atgagtaccg catgcacaag tcccggatgt acagccagtg |
| 3481 | tgtccgaatg aggcacctct ctcaagagtt tggatggctc caaatcaccc cccaggaatt |
| 3541 | cctgtgcatg aaagcactgc tactcttcag cattattcca gtggatgggc tgaaaaatca |
| 3601 | aaaattcttt gatgaacttc gaatgaacta catcaaggaa ctcgatcgta tcattgcatg |
| 3661 | caaaagaaaa aatcccacat cctgctcaag acgcttctac cagctcacca agctcctgga |
| 3721 | ctccgtgcag cctattgcga gagagctgca tcagttcact tttgacctgc taatcaagtc |
| 3781 | acacatggtg agcgtggact ttccggaaat gatggcagag atcatctctg tgcaagtgcc |
| 3841 | caagatcctt tctgggaaag tcaagcccat ctatttccac acccagtgaa gcattggaaa |
| 3901 | ccctatttcc ccaccccagc tcatgccccc tttcagatgt cttctgcctg ttataactct |
| 3961 | gcactactcc tctgcagtgc cttggggaat ttcctctatt gatgtacagt ctgtcatgaa |
| 4021 | catgttcctg aattctattt gctgggcttt ttttttctct ttctctcctt tctttttctt |
| 4081 | cttccctccc tatctaaccc tcccatggca ccttcagact ttgcttccca ttgtggctcc |
| 4141 | tatctgtgtt ttgaatggtg ttgtatgcct ttaaatctgt gatgatcctc atatggccca |
| 4201 | gtgtcaagtt gtgcttgttt acagcactac tctgtgccag ccacacaaac gtttacttat |
| 4261 | cttatgccac gggaagttta gagagctaag attatctggg gaaatcaaaa caaaaacaag |
| 4321 | caaacaaaaa aaaaaagcaa aaacaaaaca aaaaataagc caaaaaacct tgctagtgtt |
| 4381 | ttttcctcaa aaataaataa ataaataaat aaatacgtac atacatacac acatacatac |
| 4441 | aaacatatag aaatccccaa agaggccaat agtgacgaga aggtgaaaat tgcaggccca |
| 4501 | tggggagtta ctgatttttt catctcctcc ctccacggga gactttattt tctgccaatg |
| 4561 | gctattgcca ttagagggca gagtgacccc agagctgagt tgggcagggg ggtggacaga |
| 4621 | gaggagagga caaggagggc aatggagcat cagtacctgc ccacagcctt ggtccctggg |
| 4681 | ggctagactg ctcaactgtg gagcaattca ttatactgaa aatgtgcttg ttgttgaaaa |
| 4741 | tttgtctgca tgttaatgcc tcacccccaa acccttttct ctctcactct ctgcctccaa |
| 4801 | cttcagattg actttcaata gtttttctaa gacctttgaa ctgaatgttc tcttcagcca |
| 4861 | aaacttggcg acttccacag aaaagtctga ccactgagaa gaaggagagc agagatttaa |
| 4921 | ccctttgtaa ggccccattt ggatccaggt ctgctttctc atgtgtgagt cagggaggag |
| 4981 | ctggagccag aggagaagaa aatgatagct tggctgttct cctgcttagg acactgactg |
| 5041 | aatagttaaa ctctcactgc cactaccttt tccccacctt taaaagacct gaatgaagtt |
| 5101 | ttctgccaaa ctccgtgaag ccacaagcac cttatgtcct cccttcagtg ttttgtgggc |
| 5161 | ctgaatttca tcacactgca tttcagccat ggtcatcaag cctgtttgct tcttttgggc |
| 5221 | atgttcacag attctctgtt aagagccccc accaccaaga aggttagcag gccaacagct |
| 5281 | ctgacatcta tctgtagatg ccagtagtca caaagatttc ttaccaactc tcagatcgct |
| 5341 | ggagccctta gacaaactgg aaagaaggca tcaaagggat caggcaagct gggcgtcttg |
| 5401 | cccttgtccc ccagagatga taccctccca gcaagtggag aagttctcac ttccttcttt |
| 5461 | agagcagcta aaggggctac ccagatcagg gttgaagaga aaactcaatt accagggtgg |
| 5521 | gaagaatgaa ggcactagaa ccagaaaccc tgcaaatgct cttcttgtca cccagcatat |
| 5581 | ccacctgcag aagtcatgag aagagagaag gaacaaagag gagactctga ctactgaatt |
| 5641 | aaaatcttca gcggcaaagc ctaaagccag atggacacca tctggtgagt ttactcatca |
| 5701 | tcctcctctg ctgctgattc tgggctctga cattgcccat actcactcag attccccacc |
| 5761 | tttgttgctg cctcttagtc agagggaggc caaaccattg agactttcta cagaaccatg |
| 5821 | gcttctttcg gaaaggtctg gttggtgtgg ctccaatact ttgccaccca tgaactcagg |
| 5881 | gtgtgccctg ggacactggt tttatatagt cttttggcac acctgtgttc tgttgacttc |
| 5941 | gttcttcaag cccaagtgca agggaaaatg tccacctact ttctcatctt ggcctctgcc |
| 6001 | tccttactta gctcttaatc tcatctgttg aactcaagaa atcaagggcc agtcatcaag |
| 6061 | ctgcccattt taattgattc actctgtttg ttgagaggat agtttctgag tgacatgata |
| 6121 | tgatccacaa gggtttcctt ccctgatttc tgcattgata ttaatagcca aacgaacttc |
| 6181 | aaaacagctt taaataacaa gggagagggg aacctaagat gagtaatatg ccaatccaag |
| 6241 | actgctggag aaaactaaag ctgacaggtt ccctttttgg ggtgggatag acatgttctg |
| 6301 | gttttcttta ttattacaca atctggctca tgtacaggat cacttttagc tgttttaaac |
| 6361 | agaaaaaaat atccaccact cttttcagtt acactaggtt acattttaat aggtccttta |
| 6421 | catctgtttt ggaatgattt tcatcttttg tgatacacag attgaattat atcattttca |
| 6481 | tatctctcct tgtaaatact agaagctctc ctttacattt ctctatcaaa tttttcatct |
| 6541 | ttatgggttt cccaattgtg actcttgtct tcatgaatat atgtttttca tttgcaaaag |
| 6601 | ccaaaaatca gtgaaacagc agtgtaatta aaagcaacaa ctggattact ccaaatttcc |
| 6661 | aaatgacaaa actagggaaa aatagcctac acaagccttt aggcctactc tttctgtgct |
| 6721 | tgggtttgag tgaacaaagg agattttagc ttggctctgt tctcccatgg atgaaaggag |
| 6781 | gaggattttt tttttctttt ggccattgat gttctagcca atgtaattga cagaagtctc |
| 6841 | attttgcatg cgctctgctc tacaaacaga gttggtatgg ttggtatact gtactcacct |
| 6901 | gtgagggact ggccactcag acccacttag ctggtgagct agaagatgag gatcactcac |
| 6961 | tggaaaagtc acaaggacca tctccaaaca agttggcagt gctcgatgtg gacgaagagt |
| 7021 | gaggaagaga aaaagaagga gcaccaggga gaaggctccg tctgtgctgg gcagcagaca |
| 7081 | gctgccagga tcacgaactc tgtagtcaaa gaaaagagtc gtgtggcagt ttcagctctc |
| 7141 | gttcattggg cagctcgcct aggcccagcc tctgagctga catgggagtt gttggattct |
| 7201 | ttgtttcata gctttttcta tgccataggc aatattgttg ttcttggaaa gtttattatt |
| 7261 | tttttaactc ccttactctg agaaagggat attttgaagg actgtcatat atctttgaaa |
| 7321 | aaagaaaatc tgtaatacat atatttttat gtatgttcac tggcactaaa aaatatagag |
| 7381 | agcttcattc tgtcctttgg gtagttgctg aggtaattgt ccaggttgaa aaataatgtg |
| 7441 | ctgatgctag agtccctctc tgtccatact ctacttctaa atacatatag gcatacatag |
| 7501 | caagttttat ttgacttgta ctttaagaga aaatatgtcc accatccaca tgatgcacaa |
| 7561 | atgagctaac attgagcttc aagtagcttc taagtgtttg tttcattagg cacagcacag |
| 7621 | atgtggcctt tccccccttc tctcccttga tatctggcag ggcataaagg cccaggccac |
| 7681 | ttcctctgcc ccttcccagc cctgcaccaa agctgcattt caggagactc tctccagaca |
| 7741 | gcccagtaac tacccgagca tggcccctgc atagccctgg aaaaataaga ggctgactgt |
| 7801 | ctacgaatta tcttgtgcca gttgcccagg tgagagggca ctgggccaag ggagtggttt |
| 7861 | tcatgtttga cccactacaa ggggtcatgg gaatcaggaa tgccaaagca ccagatcaaa |
| 7921 | tccaaaactt aaagtcaaaa taagccattc agcatgttca gtttcttgga aaaggaagtt |
| 7981 | tctacccctg atgcctttgt aggcagatct gttctcacca ttaatctttt tgaaaatctt |
| 8041 | ttaaagcagt ttttaaaaag agagatgaaa gcatcacatt atataaccaa agattacatt |
| 8101 | gtacctgcta agataccaaa attcataagg gcaggggggg agcaagcatt agtgcctctt |
| 8161 | tgataagctg tccaaagaca gactaaagga ctctgctggt gactgactta taagagcttt |
| 8221 | gtgggttttt ttttccctaa taatatacat gtttagaaga attgaaaata atttcgggaa |
| 8281 | aatgggatta tgggtccttc actaagtgat tttataagca gaactggctt tccttttctc |
| 8341 | tagtagttgc tgagcaaatt gttgaagctc catcattgca tggttggaaa tggagctgtt |
| 8401 | cttagccact gtgtttgcta gtgcccatgt tagcttatct gaagatgtga aacccttgct |
| 8461 | gataagggag catttaaagt actagatttt gcactagagg gacagcaggc agaaatcctt |
| 8521 | atttctgccc actttggatg gcacaaaaag ttatctgcag ttgaaggcag aaagttgaaa |
| 8581 | tacattgtaa atgaatattt gtatccatgt ttcaaaattg aaatatatat atatatatat |
| 8641 | atatatatat atatatatat atagtgtgtg tgtgtgttct gatagcttta actttctctg |
| 8701 | catctttata tttggttcca gatcacacct gatgccatgt acttgtgaga gaggatgcag |
| 8761 | ttttgttttg gaagctctct cagaacaaac aagacacctg gattgatcag ttaactaaaa |
| 8821 | gttttctccc ctattgggtt tgacccacag gtcctgtgaa ggagcagagg gataaaaaga |
| 8881 | gtagaggaca tgatacattg tactttacta gttcaagaca gatgaatgtg gaaagcataa |
| 8941 | aaactcaatg gaactgactg agatttacca cagggaaggc ccaaacttgg ggccaaaagc |
| 9001 | ctacccaagt gattgaccag tggcccccta atgggacctg agctgttgga agaagagaac |
| 9061 | tgttccttgg tcttcaccat ccttgtgaga gaagggcagt ttcctgcatt ggaacctgga |
| 9121 | gcaagcgctc tatctttcac acaaattccc tcacctgaga ttgaggtgct cttgttactg |
| 9181 | ggtgtctgtg tgctgtaatt ctggttttgg atatgttctg taaagatttt gacaaatgaa |
| 9241 | aatgtgtttt tctctgttaa aacttgtcag agtactagaa gttgtatctc tgtaggtgca |
| 9301 | ggtccatttc tgcccacagg tagggtgttt ttctttgatt aagagattga cacttctgtt |
| 9361 | gcctaggacc tcccaactca accatttcta ggtgaaggca gaaaaatcca cattagttac |
| 9421 | tcctcttcag acatttcagc tgagataaca aatcttttgg aattttttca cccatagaaa |
| 9481 | gagtggtaga tatttgaatt tagcaggtgg agtttcatag taaaaacagc ttttgactca |
| 9541 | gctttgattt atcctcattt gatttggcca gaaagtaggt aatatgcatt gattggcttc |
| 9601 | tgattccaat tcagtatagc aaggtgctag gttttttcct ttccccacct gtctcttagc |
| 9661 | ctggggaatt aaatgagaag ccttagaatg ggtggccctt gtgacctgaa acacttccca |
| 9721 | cataagctac ttaacaagat tgtcatggag ctgcagattc cattgcccac caaagactag |
| 9781 | aacacacaca tatccataca ccaaaggaaa gacaattctg aaatgctgtt tctctggtgg |
| 9841 | ttccctctct ggctgctgcc tcacagtatg ggaacctgta ctctgcagag gtgacaggcc |
| 9901 | agatttgcat tatctcacaa ccttagccct tggtgctaac tgtcctacag tgaagtgcct |
| 9961 | ggggggttgt cctatcccat aagccacttg gatgctgaca gcagccacca tcagaatgac |
| 10021 | ccacgcaaaa aaaagaaaaa aaaaattaaa aagtcccctc acaacccagt gacacctttc |
| 10081 | tgctttcctc tagactggaa cattgattac ggagtgcctc agacatgaca ttcttgtgct |
| 10141 | gtccttggaa ttaatctggc agcaggaggc agcagactat gtaaacagac ataaaaatta |
| 10201 | attttcaata ttgaaggaaa aaagaaataa gaagagagac agaaagaaac catcacacaa |
| 10261 | agattttctt aaaagaaaca attttgcttc aaatctcttt agatggggct catttctcac |
| 10321 | ggtggcactt ggcctccact gggcagcagc accagctcca agcgctagtc ttctgttctc |
| 10381 | tttttgtaat cttggaatct tttgttgctc taaatacaat taaaaatggc agaaacttgt |
| 10441 | ttgttggact acatgtgtga ctttgggtct gtctctgcct ctgctttcac aaatgtcatc |
| 10501 | cattgtgtaa aatattggct tactggtctc ccagctaaaa cttggccaca tcccctgtta |
| 10561 | tggctgcagc atcgagttat tgttaacaaa gagacccaac aaaagctgct aatgtcctct |
| 10621 | tatcattgtt gttaatttgt taaaacataa agaaatctaa aatttca |

SEQ ID NO: 50 Human AR Isoform 2 Amino Acid Sequence (NP_001011645.1)

| | |
|---|---|
| 1 | milwlhslet ardhvlpidy yfppqktcli cgdeasgchy galtcgsckv ffkraaegkq |
| 61 | kylcasrndc tidkfrrknc pscrlrkcye agmtlgarkl kklgnlklqe egeassttsp |
| 121 | teettqkltv shiegyecqp iflnvleaie pgvvcaghdn nqpdsfaall sslnelgerq |
| 181 | lvhvvkwaka lpgfrnlhvd dqmaviqysw mglmvfamgw rsftnvnsrm lyfapdlvfn |
| 241 | eyrmhksrmy sqcvrmrhls qefgwlqitp qeflcmkall lfsiipvdgl knqkffdelr |
| 301 | mnyikeldri iackrknpts csrrfyqltk lldsvqpiar elhqftfdll ikshmvsvdf |
| 361 | pemmaeiisv qvpkilsgkv kpiyfhtq |

SEQ ID NO: 51 Human AR transcript variant 2 cDNA sequence (NM_001011645.3;
CDS: 2308 . . . 3474)

| | |
|---|---|
| 1 | gcggagagaa ccctctgttt tcccccactc tctctccacc tcctcctgcc ttccccaccc |
| 61 | cgagtgcgga gccagagatc aaaagatgaa aaggcagtca ggtcttcagt agccaaaaaa |
| 121 | caaaacaaac aaaaacaaaa aagccgaaat aaaagaaaaa gataataact cagttcttat |
| 181 | ttgcacctac ttcagtggac actgaatttg gaaggtggag gattttgttt ttttctttta |
| 241 | agatctgggc atcttttgaa tctacccttc aagtattaag agacagactg tgagcctagc |
| 301 | agggcagatc ttgtccaccg tgtgtcttct tctgcacgag actttgaggc tgtcagagcg |
| 361 | ctttttgcgt ggttgctccc gcaagtttcc ttctctggag cttcccgcag gtgggcagct |
| 421 | agctgcagcg actaccgcat catcacagcc tgttgaactc ttctgagcaa gagaagggga |
| 481 | ggcggggtaa gggaagtagg tggaagattc agccaagctc aaggatggaa gtgcagttag |
| 541 | ggctgggaag ggtctaccct cggccgccgt ccaagaccta ccgaggagct ttccagaatc |
| 601 | tgttccagag cgtgcgcgaa gtgatccaga acccgggccc caggcaccca gaggccgcga |
| 661 | gcgcagcacc tcccggcgcc agtttgctgc tgctgcagca gcagcagcag cagcagcagc |
| 721 | agcagcagca gcagcagcag cagcagcagc agcagcagca gcaagagact agccccaggc |
| 781 | agcagcagca gcagcagggt gaggatggtt ctccccaagc ccatcgtaga ggccccacag |
| 841 | gctacctggt cctggatgag gaacagcaac cttcacagcc gcagtcggcc ctggagtgcc |
| 901 | accccgagag aggttgcgtc ccagagcctg gagccgccgt ggccgccagc aaggggctgc |
| 961 | cgcagcagct gccagcacct ccggacgagg atgactcagc tgccccatcc acgttgtccc |
| 1021 | tgctgggccc cactttcccc ggcttaagca gctgctccgc tgaccttaaa gacatcctga |
| 1081 | gcgaggccag caccatgcaa ctccttcagc aacagcagca ggaagcagta tccgaaggca |
| 1141 | gcagcagcgg gagagcgagg gaggcctcgg gggctcccac ttcctccaag gacaattact |
| 1201 | tagggggcac ttcgaccatt tctgacaacg ccaaggagtt gtgtaaggca gtgtcggtgt |
| 1261 | ccatgggcct gggtgtggag gcgttggagc atctgagtcc aggggaacag cttcgggggg |
| 1321 | attgcatgta cgccccactt ttgggagttc cacccgctgt gcgtcccact ccttgtgccc |
| 1381 | cattggccga atgcaaaggt tctctgctag acgacagcgc aggcaagagc actgaagata |
| 1441 | ctgctgagta ttcccctttc aagggaggtt acaccaaagg gctagaaggc gagagcctag |
| 1501 | gctgctctgg cagcgctgca gcagggagct ccgggacact tgaactgccg tctaccctgt |
| 1561 | ctctctacaa gtccggagca ctggacgagg cagctgcgta ccagagtcgc gactactaca |
| 1621 | actttccact ggctctggcc ggaccgccgc cccctccgcc gcctccccat ccccacgctc |
| 1681 | gcatcaagct ggagaacccg ctggactacg gcagcgcctg ggcggctgcg gcggcgcagt |
| 1741 | gccgctatgg ggacctggcg agcctgcatg gcgcgggtgc agcgggaccc ggttctgggt |
| 1801 | caccctcagc cgccgcttcc tcatcctggc acactctctt cacagccgaa gaaggccagt |
| 1861 | tgtatggacc gtgtggtggt ggtgggggtg gtggcggcgg cggcggcggc ggcggcggcg |
| 1921 | gcggcggcgg cggcggcggc ggcgaggcgg gagctgtagc cccctacggc tacactcggc |
| 1981 | cccctcaggg gctggcgggc caggaaagcg acttcaccgc acctgatgtg tggtaccctg |
| 2041 | gcggcatggt gagcagagtg ccctatccca gtcccacttg tgtcaaaagc gaaatgggcc |
| 2101 | cctggatgga tagctactcc ggaccttacg gggacatgcg taggtgctgc gagcagagag |
| 2161 | ggattcctcg gaggtcatct gttccatctt cttgcctatg caaatgcctg cctgaagctg |
| 2221 | ctggaggctg gctttgtacc ggactttgta cagggaacca gggaaacgaa tgcagagtgc |
| 2281 | tcctgacatt gcctgtcact ttttcccatg atactctggc ttcacagttt ggagactgcc |
| 2341 | agggaccatg ttttgcccat tgactattac tttccacccc agaagacctg cctgatctgt |
| 2401 | ggagatgaag cttctgggtg tcactatgga gctctcacat gtggaagctg caaggtcttc |
| 2461 | ttcaaaagag ccgctgaagg gaaacagaag tacctgtgcg ccagcagaaa tgattgcact |
| 2521 | attgataaat tccgaaggaa aaattgtcca tcttgtcgtc ttcggaaatg ttatgaagca |
| 2581 | gggatgactc tgggagcccg gaagctgaag aaacttggta atctgaaact acaggaggaa |
| 2641 | ggagaggctt ccagcaccac cagccccact gaggagacaa cccagaagct gacagtgtca |
| 2701 | cacattgaag gctatgaatg tcagcccatc tttctgaatg tcctggaagc cattgagcca |
| 2761 | ggtgtagtgt gtgctggaca cgacaacaac cagcccgact cctttgcagc cttgctctct |
| 2821 | agcctcaatg aactgggaga gagacagctt gtacacgtgg tcaagtgggc caaggccttg |
| 2881 | cctggcttcc gcaacttaca cgtggacgac cagatggctg tcattcagta ctcctggatg |
| 2941 | gggctcatgg tgtttgccat gggctggcga tccttcacca atgtcaactc caggatgctc |
| 3001 | tacttcgccc ctgatctggt tttcaatgag taccgcatgc acaagtcccg gatgtacagc |
| 3061 | cagtgtgtcc gaatgaggca cctctctcaa gagtttggat ggctccaaat caccccccag |
| 3121 | gaattcctgt gcatgaaagc actgctactc ttcagcatta ttccagtgga tgggctgaaa |
| 3181 | aatcaaaaat tctttgatga acttcgaatg aactacatca aggaactcga tcgtatcatt |
| 3241 | gcatgcaaaa gaaaaaatcc cacatcctgc tcaagacgct tctaccagct caccaagctc |
| 3301 | ctggactccg tgcagcctat tgcgagagag ctgcatcagt tcacttttga cctgctaatc |
| 3361 | aagtcacaca tggtgagcgt ggactttccg gaaatgatgg cagagatcat ctctgtgcaa |
| 3421 | gtgcccaaga tcctttctgg gaaagtcaag cccatctatt tccacaccca gtgaagcatt |
| 3481 | ggaaacccta tttccccacc ccagctcatg ccccctttca gatgtcttct gcctgttata |
| 3541 | actctgcact actcctctgc agtgccttgg ggaatttcct ctattgatgt acagtctgtc |
| 3601 | atgaacatgt tcctgaattc tatttgctgg gctttttttt tctctttctc tcctttcttt |
| 3661 | ttcttcttcc ctccctatct aaccctccca tggcaccttc agactttgct tcccattgtg |
| 3721 | gctcctatct gtgttttgaa tggtgttgta tgcctttaaa tctgtgatga tcctcatatg |
| 3781 | gcccagtgtc aagttgtgct tgtttacagc actactctgt gccagccaca caaacgttta |
| 3841 | cttatcttat gccacgggaa gtttagagag ctaagattat ctggggaaat caaaacaaaa |
| 3901 | acaagcaaac aaaaaaaaaa agcaaaaaca aaacaaaaaa taagccaaaa aaccttgcta |
| 3961 | gtgttttttc ctcaaaaata aataaataaa taaataaata cgtacataca tacacacata |
| 4021 | catacaaaca tatagaaatc cccaaagagg ccaatagtga cgagaaggtg aaaattgcag |
| 4081 | gcccatgggg agttactgat tttttcatct cctccctcca cgggagactt tattttctgc |
| 4141 | caatggctat tgccattaga gggcagagtg accccagagc tgagttgggc aggggggtgg |
| 4201 | acagagagga gaggacaagg agggcaatgg agcatcagta cctgcccaca gccttggtcc |
| 4261 | ctgggggcta gactgctcaa ctgtggagca attcattata ctgaaaatgt gcttgttgtt |
| 4321 | gaaaatttgt ctgcatgtta atgcctcacc cccaaaccct tttctctctc actctctgcc |
| 4381 | tccaacttca gattgacttt caatagtttt tctaagacct ttgaactgaa tgttctcttc |
| 4441 | agccaaaact tggcgacttc cacagaaaag tctgaccact gagaagaagg agagcagaga |
| 4501 | tttaaccctt tgtaaggccc catttggatc caggtctgct ttctcatgtg tgagtcaggg |
| 4561 | aggagctgga gccagaggag aagaaaatga tagcttggct gttctcctgc ttaggacact |
| 4621 | gactgaatag ttaaactctc actgccacta ccttttcccc acctttaaaa gacctgaatg |
| 4681 | aagttttctg ccaaactccg tgaagccaca agcaccttat gtcctccctt cagtgttttg |
| 4741 | tgggcctgaa tttcatcaca ctgcatttca gccatggtca tcaagcctgt ttgcttcttt |
| 4801 | tgggcatgtt cacagattct ctgttaagag cccccaccac caagaaggtt agcaggccaa |
| 4861 | cagctctgac atctatctgt agatgccagt agtcacaaag atttcttacc aactctcaga |
| 4921 | tcgctggagc ccttagacaa actggaaaga aggcatcaaa gggatcaggc aagctgggcg |
| 4981 | tcttgccctt gtcccccaga gatgataccc tcccagcaag tggagaagtt ctcacttcct |
| 5041 | tctttagagc agctaaaggg gctacccaga tcagggttga agagaaaact caattaccag |
| 5101 | ggtgggaaga atgaaggcac tagaaccaga aaccctgcaa atgctcttct tgtcacccag |
| 5161 | catatccacc tgcagaagtc atgagaagag agaaggaaca aagaggagac tctgactact |
| 5221 | gaattaaaat cttcagcggc aaagcctaaa gccagatgga caccatctgg tgagtttact |
| 5281 | catcatcctc ctctgctgct gattctgggc tctgacattg cccatactca ctcagattcc |
| 5341 | ccacctttgt tgctgcctct tagtcagagg gaggccaaac cattgagact ttctacagaa |
| 5401 | ccatggcttc tttcggaaag gtctggttgg tgtggctcca atactttgcc acccatgaac |
| 5461 | tcagggtgtg ccctgggaca ctggttttat atagtctttt ggcacacctg tgttctgttg |
| 5521 | acttcgttct tcaagcccaa gtgcaaggga aaatgtccac ctactttctc atcttggcct |
| 5581 | ctgcctcctt acttagctct taatctcatc tgttgaactc aagaaatcaa gggccagtca |
| 5641 | tcaagctgcc cattttaatt gattcactct gtttgttgag aggatagttt ctgagtgaca |
| 5701 | tgatatgatc cacaagggtt tccttccctg atttctgcat tgatattaat agccaaacga |
| 5761 | acttcaaaac agctttaaat aacaagggag aggggaacct aagatgagta atatgccaat |
| 5821 | ccaagactgc tggagaaaac taaagctgac aggttccctt tttggggtgg gatagacatg |
| 5881 | ttctggtttt ctttattatt acacaatctg gctcatgtac aggatcactt ttagctgttt |
| 5941 | taaacagaaa aaaatatcca ccactctttt cagttacact aggttacatt ttaataggtc |
| 6001 | ctttacatct gttttggaat gattttcatc ttttgtgata cacagattga attatatcat |
| 6061 | tttcatatct ctccttgtaa atactagaag ctctccttta catttctcta tcaaattttt |
| 6121 | catctttatg ggtttcccaa ttgtgactct tgtcttcatg aatatatgtt tttcatttgc |
| 6181 | aaaagccaaa aatcagtgaa acagcagtgt aattaaaagc aacaactgga ttactccaaa |
| 6241 | tttccaaatg acaaaactag ggaaaaatag cctacacaag cctttaggcc tactctttct |
| 6301 | gtgcttgggt ttgagtgaac aaaggagatt ttagcttggc tctgttctcc catggatgaa |
| 6361 | aggaggagga tttttttttt cttttggcca ttgatgttct agccaatgta attgacagaa |
| 6421 | gtctcatttt gcatgcgctc tgctctacaa acagagttgg tatggttggt atactgtact |
| 6481 | cacctgtgag ggactggcca ctcagaccca cttagctggt gagctagaag atgaggatca |
| 6541 | ctcactggaa aagtcacaag gaccatctcc aaacaagttg gcagtgctcg atgtggacga |
| 6601 | agagtgagga agagaaaaag aaggagcacc agggagaagg ctccgtctgt gctgggcagc |
| 6661 | agacagctgc caggatcacg aactctgtag tcaaagaaaa gagtcgtgtg gcagtttcag |
| 6721 | ctctcgttca ttgggcagct cgcctaggcc cagcctctga gctgacatgg gagttgttgg |
| 6781 | attctttgtt tcatagcttt ttctatgcca taggcaatat tgttgttctt ggaaagttta |
| 6841 | ttattttttt aactccctta ctctgagaaa gggatatttt gaaggactgt catatatctt |
| 6901 | tgaaaaaaga aaatctgtaa tacatatatt tttatgtatg ttcactggca ctaaaaaata |
| 6961 | tagagagctt cattctgtcc tttgggtagt tgctgaggta attgtccagg ttgaaaaata |
| 7021 | atgtgctgat gctagagtcc ctctctgtcc atactctact tctaaataca tataggcata |
| 7081 | catagcaagt tttatttgac ttgtacttta agagaaaata tgtccaccat ccacatgatg |
| 7141 | cacaaatgag ctaacattga gcttcaagta gcttctaagt gtttgtttca ttaggcacag |
| 7201 | cacagatgtg gcctttcccc ccttctctcc cttgatatct ggcagggcat aaaggcccag |
| 7261 | gccacttcct ctgccccttc ccagccctgc accaaagctg catttcagga gactctctcc |
| 7321 | agacagccca gtaactaccc gagcatggcc cctgcatagc cctggaaaaa taagaggctg |
| 7381 | actgtctacg aattatcttg tgccagttgc ccaggtgaga gggcactggg ccaagggagt |
| 7441 | ggttttcatg tttgacccac tacaaggggt catgggaatc aggaatgcca aagcaccaga |
| 7501 | tcaaatccaa aacttaaagt caaaataagc cattcagcat gttcagtttc ttggaaaagg |
| 7561 | aagtttctac ccctgatgcc tttgtaggca gatctgttct caccattaat ctttttgaaa |
| 7621 | atcttttaaa gcagttttta aaaagagaga tgaaagcatc acattatata accaaagatt |
| 7681 | acattgtacc tgctaagata ccaaaattca taagggcagg gggggagcaa gcattagtgc |
| 7741 | ctctttgata agctgtccaa agacagacta aaggactctg ctggtgactg acttataaga |
| 7801 | gctttgtggg tttttttttc cctaataata tacatgttta gaagaattga aaataatttc |
| 7861 | gggaaaatgg gattatgggt ccttcactaa gtgattttat aagcagaact ggctttcctt |
| 7921 | ttctctagta gttgctgagc aaattgttga agctccatca ttgcatggtt ggaaatggag |
| 7981 | ctgttcttag ccactgtgtt tgctagtgcc catgttagct tatctgaaga tgtgaaaccc |
| 8041 | ttgctgataa gggagcattt aaagtactag attttgcact agagggacag caggcagaaa |
| 8101 | tccttatttc tgcccacttt ggatggcaca aaaagttatc tgcagttgaa ggcagaaagt |
| 8161 | tgaaatacat tgtaaatgaa tatttgtatc catgtttcaa aattgaaata tatatatata |
| 8221 | tatatatata tatatatata tatatatagt gtgtgtgtgt gttctgatag ctttaacttt |
| 8281 | ctctgcatct ttatatttgg ttccagatca cacctgatgc catgtacttg tgagagagga |
| 8341 | tgcagttttg ttttggaagc tctctcagaa caaacaagac acctggattg atcagttaac |
| 8401 | taaaagtttt ctcccctatt gggtttgacc cacaggtcct gtgaaggagc agagggataa |
| 8461 | aaagagtaga ggacatgata cattgtactt tactagttca agacagatga atgtggaaag |
| 8521 | cataaaaact caatggaact gactgagatt taccacaggg aaggcccaaa cttggggcca |
| 8581 | aaagcctacc caagtgattg accagtggcc ccctaatggg acctgagctg ttggaagaag |
| 8641 | agaactgttc cttggtcttc accatccttg tgagagaagg gcagtttcct gcattggaac |
| 8701 | ctggagcaag cgctctatct ttcacacaaa ttccctcacc tgagattgag gtgctcttgt |
| 8761 | tactgggtgt ctgtgtgctg taattctggt tttggatatg ttctgtaaag attttgacaa |
| 8821 | atgaaaatgt gtttttctct gttaaaactt gtcagagtac tagaagttgt atctctgtag |
| 8881 | gtgcaggtcc atttctgccc acaggtaggg tgtttttctt tgattaagag attgacactt |
| 8941 | ctgttgccta ggacctccca actcaaccat ttctaggtga aggcagaaaa atccacatta |
| 9001 | gttactcctc ttcagacatt tcagctgaga taacaaatct tttggaattt tttcacccat |
| 9061 | agaaagagtg gtagatattt gaatttagca ggtggagttt catagtaaaa acagcttttg |
| 9121 | actcagcttt gatttatcct catttgattt ggccagaaag taggtaatat gcattgattg |
| 9181 | gcttctgatt ccaattcagt atagcaaggt gctaggtttt ttcctttccc cacctgtctc |
| 89241 | ttagcctggg gaattaaatg agaagcctta gaatgggtgg cccttgtgac ctgaaacact |
| 9301 | tcccacataa gctacttaac aagattgtca tggagctgca gattccattg cccaccaaag |
| 9361 | actagaacac acacatatcc atacaccaaa ggaaagacaa ttctgaaatg ctgtttctct |
| 9421 | ggtggttccc tctctggctg ctgcctcaca gtatgggaac ctgtactctg cagaggtgac |
| 9481 | aggccagatt tgcattatct cacaacctta gcccttggtg ctaactgtcc tacagtgaag |
| 9541 | tgcctggggg gttgtcctat cccataagcc acttggatgc tgacagcagc caccatcaga |
| 9601 | atgacccacg caaaaaaaag aaaaaaaaaa ttaaaaagtc ccctcacaac ccagtgacac |
| 9661 | ctttctgctt tcctctagac tggaacattg attagggagt gcctcagaca tgacattctt |
| 9721 | gtgctgtcct tggaattaat ctggcagcag gagggagcag actatgtaaa cagagataaa |
| 9781 | aattaatttt caatattgaa ggaaaaaaga aataagaaga gagagagaaa gaaagcatca |
| 9841 | cacaaagatt ttcttaaaag aaacaatttt gcttgaaatc tctttagatg gggctcattt |
| 9901 | ctcacggtgg cacttggcct ccactgggca gcaggaccag ctccaagcgc tagtgttctg |
| 9961 | ttctcttttt gtaatcttgg aatcttttgt tgctctaaat acaattaaaa atggcagaaa |
| 10021 | cttgtttgtt ggactacatg tgtgactttg ggtctgtctc tgcctctgct ttcagaaatg |
| 10081 | tcatccattg tgtaaaatat tggcttactg gtctgccagc taaaacttgg ccacatcccc |
| 10141 | tgttatggct gcaggatcga gttattgtta acaaagagac ccaagaaaag ctgctaatgt |
| 10201 | cctcttatca ttgttgttaa tttgttaaaa cataaagaaa tctaaaattt caaaaaa |

SEQ ID NO: 52 Human AR Isoform 3 Amino Acid Sequence (NP_001334990.1)

| | |
|---|---|
| 1 | mevqlglgrv yprppsktyr gafqnlfqsv reviqnpgpr hpeaasaapp gasllllqqq |
| 61 | qqqqqqqqqq qqqqqqqqqq etsprqqqqq qgedgspqah rrgptgylvl deeqqpsqpq |
| 121 | salechperg cvpepgaava askglpqqlp appdeddsaa pstlsllgpt fpglsscsad |
| 181 | lkdilseast mqllqqqqqe avsegsssgr areasgapts skdnylggts tisdnakelc |
| 241 | kavsvsmglg vealehlspg eqlrgdcmya pllgvppavr ptpcaplaec kgsllddsag |
| 301 | kstedtaeys pfkggytkgl egeslgcsgs aaagssgtle lpstlslyks galdeaaayq |
| 361 | srdyynfpla lagppppppp phpharikle npldygsawa aaaaqcrygd laslhgagaa |
| 421 | gpgsgspsaa assswhtlft aeegqlygpc gggggggggg gggggggggg gggeagavap |
| 481 | ygytrppqgl agqesdftap dvwypggmvs rvpypsptcv ksemgpwmds ysgpygdmrl |
| 541 | etardhvlpi dyyfppqktc licgdeasgc hygaltcgsc kvffkraaeg kqkylcasrn |
| 601 | dctidkfrrk ncpscrlrkc yeagmtlgek frvgnckhlk mtrp |

SEQ ID NO: 53 Human AR transcript variant 3 cDNA sequence (NM_001348061.1;
CDS: 525 . . . 2459)

| | |
|---|---|
| 1 | gcggagagaa ccctctgttt tcccccactc tctctccacc tcctcctgcc ttccccaccc |
| 61 | cgagtgcgga gccagagatc aaaagatgaa aaggcagtca ggtcttcagt agccaaaaaa |
| 121 | caaaacaaac aaaaacaaaa aagccgaaat aaaagaaaaa gataataact cagttcttat |
| 181 | ttgcacctac ttcagtggac actgaatttg gaaggtggag gattttgttt ttttctttta |
| 241 | agatctgggc atcttttgaa tctacccttc aagtattaag agacagactg tgagcctagc |
| 301 | agggcagatc ttgtccaccg tgtgtcttct tctgcacgag actttgaggc tgtcagagcg |
| 361 | ctttttgcgt ggttgctccc gcaagtttcc ttctctggag cttcccgcag gtgggcagct |
| 421 | agctgcagcg actaccgcat catcacagcc tgttgaactc ttctgagcaa gagaagggga |
| 481 | ggcggggtaa gggaagtagg tggaagattc agccaagctc aaggatggaa gtgcagttag |
| 541 | ggctgggaag ggtctaccct cggccgccgt ccaagaccta ccgaggagct ttccagaatc |
| 601 | tgttccagag cgtgcgcgaa gtgatccaga acccgggccc caggcaccca gaggccgcga |
| 661 | gcgcagcacc tcccggcgcc agtttgctgc tgctgcagca gcagcagcag cagcagcagc |
| 721 | agcagcagca gcagcagcag cagcagcagc agcagcagca gcaagagact agccccaggc |
| 781 | agcagcagca gcagcagggt gaggatggtt ctccccaagc ccatcgtaga ggccccacag |
| 841 | gctacctggt cctggatgag gaacagcaac cttcacagcc gcagtcggcc ctggagtgcc |
| 901 | accccgagag aggttgcgtc ccagagcctg gagccgccgt ggccgccagc aaggggctgc |
| 961 | cgcagcagct gccagcacct ccggacgagg atgactcagc tgccccatcc acgttgtccc |
| 1021 | tgctgggccc cactttcccc ggcttaagca gctgctccgc tgaccttaaa gacatcctga |
| 1081 | gcgaggccag caccatgcaa ctccttcagc aacagcagca ggaagcagta tccgaaggca |
| 1141 | gcagcagcgg gagagcgagg gaggcctcgg gggctcccac ttcctccaag gacaattact |
| 1201 | tagggggcac ttcgaccatt tctgacaacg ccaaggagtt gtgtaaggca gtgtcggtgt |
| 1261 | ccatgggcct gggtgtggag gcgttggagc atctgagtcc aggggaacag cttcgggggg |
| 1321 | attgcatgta cgccccactt ttgggagttc cacccgctgt gcgtcccact ccttgtgccc |
| 1381 | cattggccga atgcaaaggt tctctgctag acgacagcgc aggcaagagc actgaagata |
| 1441 | ctgctgagta ttcccctttc aagggaggtt acaccaaagg gctagaaggc gagagcctag |
| 1501 | gctgctctgg cagcgctgca gcagggagct ccgggacact tgaactgccg tctaccctgt |
| 1561 | ctctctacaa gtccggagca ctggacgagg cagctgcgta ccagagtcgc gactactaca |
| 1621 | actttccact ggctctggcc ggaccgccgc cccctccgcc gcctccccat ccccacgctc |
| 1681 | gcatcaagct ggagaacccg ctggactacg gcagcgcctg ggcggctgcg gcggcgcagt |
| 1741 | gccgctatgg ggacctggcg agcctgcatg gcgcgggtgc agcgggaccc ggttctgggt |
| 1801 | caccctcagc cgccgcttcc tcatcctggc acactctctt cacagccgaa gaaggccagt |
| 1861 | tgtatggacc gtgtggtggt ggtgggggtg gtggcggcgg cggcggcggc ggcggcggcg |
| 1921 | gcggcggcgg cggcggcggc ggcgaggcgg gagctgtagc cccctacggc tacactcggc |
| 1981 | cccctcaggg gctggcgggc caggaaagcg acttcaccgc acctgatgtg tggtaccctg |
| 2041 | gcggcatggt gagcagagtg ccctatccca gtcccacttg tgtcaaaagc gaaatgggcc |
| 2101 | cctggatgga tagctactcc ggaccttacg gggacatgcg tttggagact gccagggacc |
| 2161 | atgttttgcc cattgactat tactttccac cccagaagac ctgcctgatc tgtggagatg |
| 2221 | aagcttctgg gtgtcactat ggagctctca catgtggaag ctgcaaggtc ttcttcaaaa |
| 2281 | gagccgctga agggaaacag aagtacctgt gcgccagcag aaatgattgc actattgata |
| 2341 | aattccgaag gaaaaattgt ccatcttgtc gtcttcggaa atgttatgaa gcagggatga |
| 2401 | ctctgggaga aaaattccgg gttggcaatt gcaagcatct caaaatgacc agaccctgaa |
| 2461 | gaaaggctga cttgcctcat tcaaaatgag ggctctagag ggctctagtg gatagtctgg |
| 2521 | agaaacctgg cgtctgaggc ttaggagctt aggtttttgc tcctcaacac agactttgac |
| 2581 | gttggggttg ggggctactc tcttgattgc tgactccctc cagcgggacc aatagtgttt |
| 2641 | tcctacctca cagggatgtt gtgaggacgg gctgtagaag taatagtggt taccattcat |
| 2701 | gtagttgtga gtatcatgat tattgtttcc tgtaatgtgg cttggcattg gcaaagtgct |
| 2761 | ttttgattgt tcttgatcac atatgatggg ggccaggcac tgactcaggc ggatgcagtg |
| 2821 | aagctctggc tcagtcgctt gcttttcgtg gtgtgctgcc aggaagaaac tttgctgatg |
| 2881 | ggactcaagg tgtcaccttg gacaagaagc aactgtgtct gtctgaggtt cctgtggcca |
| 2941 | tctttatttg tgtattaggc aattcgtatt tcccccttag gttctagcct tctggatccc |
| 3001 | agccagtgac ctagatctta gcctcaggcc ctgtcactga gctgaaggta gtagctgatc |
| 3061 | cacagaagtt cagtaaacaa ggaccagatt tctgcttctc caggagaaga agccagccaa |
| 3121 | cccctctctt caaacacact gagagactac agtccgactt tccctcttac atctagcctt |
| 3181 | actgtagcca cactccttga ttgctctctc acatcacatg cttctcttca tcagttgtaa |
| 3241 | gcctctcatt cttctcccaa gccagactca aatattgtat tgatgtcaaa gaagaatcac |
| 3301 | ttagagtttg gaatatcttg ttctctctct gctccatagc ttccatattg acaccagttt |
| 3361 | ctttctagtg gagaagtgga gtctgtgaag ccagggaaac acacatgtga gagtcagaag |
| 3421 | gactctccct gacttgcctg gggcctgtct ttcccacctt ctccagtctg tctaaacaca |
| 3481 | cacacacaca cacacacaca cacacacacg ctctctctct ctctcccccc ccaacacaca |
| 3541 | cacactctct ctctctctca cacacacaca catacacaca cacttctttc tctttcccct |
| 3601 | gactcagcaa cattctggag aaaagccaag gaaggacttc aggaggggag tttccccctt |
| 3661 | ctcagggcag aattttaatc tccagaccaa caagaagttc cctaatgtgg attgaaaggc |
| 3721 | taatgaggtt tatttttaac tactttctat ttgtttgaat gttgcatatt tctactagtg |
| 3781 | aaattttccc ttaataaagc cattaataca cc |

SEQ ID NO: 54 Human AR Isoform 4 Amino Acid Sequence (NP_001334992.1)

| | |
|---|---|
| 1 | mevqlglgrv yprppsktyr gafqnlfqsv reviqnpgpr hpeaasaapp gasllllqqq |
| 61 | qqqqqqqqqq qqqqqqqqqq etsprqqqqq qgedgspqah rrgptgylvl deeqqpsqpq |
| 121 | salechperg cvpepgaava askglpqqlp appdeddsaa pstlsllgpt fpglsscsad |
| 181 | lkdilseast mqllqqqqqe avsegsssgr areasgapts skdnylggts tisdnakelc |
| 241 | kavsvsmglg vealehlspg eqlrgdcmya pllgvppavr ptpcaplaec kgsllddsag |
| 301 | kstedtaeys pfkggytkgl egeslgcsgs aaagssgtle lpstlslyks galdeaaayq |
| 361 | srdyynfpla lagppppppp phpharikle npldygsawa aaaaqcrygd laslhgagaa |
| 421 | gpgsgsgsaa assswhtlft aeegqlygpc gggggggggg gggggggggg gggeagavap |
| 481 | ygytrppqgl agqesdftap dvwypggmvs rvpypsptcv ksemgpwmds ysgpygdmrl |
| 541 | etardhvlpi dyyfppqktc licgdeasgc hygaltcgsc kvffkraaeg kqkylcasrn |
| 601 | dctidkfrrk ncpscrlrkc yeagmtlgaa vvvserilrv fgvsewlp |

SEQ ID NO: 55 Human AR transcript variant 4 cDNA sequence (NM_001348063.1;
CDS: 525 . . . 2471)

| | |
|---|---|
| 1 | gcggagagaa ccctctgttt tcccccactc tctctccacc tcctcctgcc ttccccaccc |
| 61 | cgagtgcgga gccagagatc aaaagatgaa aaggcagtca ggtcttcagt agccaaaaaa |
| 121 | caaaacaaac aaaaacaaaa aagccgaaat aaaagaaaaa gataataact cagttcttat |
| 181 | ttgcacctac ttcagtggac actgaatttg gaaggtggag gattttgttt ttttctttta |
| 241 | agatctgggc atcttttgaa tctacccttc aagtattaag agacagactg tgagcctagc |
| 301 | agggcagatc ttgtccaccg tgtgtcttct tctgcacgag actttgaggc tgtcagagcg |
| 361 | ctttttgcgt ggttgctccc gcaagtttcc ttctctggag cttcccgcag gtgggcagct |
| 421 | agctgcagcg actaccgcat catcacagcc tgttgaactc ttctgagcaa gagaagggga |
| 481 | ggcggggtaa gggaagtagg tggaagattc agccaagctc aaggatggaa gtgcagttag |
| 541 | ggctgggaag ggtctaccct cggccgccgt ccaagaccta ccgaggagct ttccagaatc |
| 601 | tgttccagag cgtgcgcgaa gtgatccaga acccgggccc caggcaccca gaggccgcga |
| 661 | gcgcagcacc tcccggcgcc agtttgctgc tgctgcagca gcagcagcag cagcagcagc |
| 721 | agcagcagca gcagcagcag cagcagcagc agcagcagca gcaagagact agccccaggc |
| 781 | agcagcagca gcagcagggt gaggatggtt ctccccaagc ccatcgtaga ggccccacag |
| 841 | gctacctggt cctggatgag gaacagcaac cttcacagcc gcagtcggcc ctggagtgcc |
| 901 | accccgagag aggttgcgtc ccagagcctg gagccgccgt ggccgccagc aaggggctgc |
| 961 | cgcagcagct gccagcacct ccggacgagg atgactcagc tgccccatcc acgttgtccc |
| 1021 | tgctgggccc cactttcccc ggcttaagca gctgctccgc tgaccttaaa gacatcctga |
| 1081 | gcgaggccag caccatgcaa ctccttcagc aacagcagca ggaagcagta tccgaaggca |
| 1141 | gcagcagcgg gagagcgagg gaggcctcgg gggctcccac ttcctccaag gacaattact |
| 1201 | tagggggcac ttcgaccatt tctgacaacg ccaaggagtt gtgtaaggca gtgtcggtgt |
| 1261 | ccatgggcct gggtgtggag gcgttggagc atctgagtcc aggggaacag cttcgggggg |
| 1321 | attgcatgta cgccccactt ttgggagttc cacccgctgt gcgtcccact ccttgtgccc |
| 1381 | cattggccga atgcaaaggt tctctgctag acgacagcgc aggcaagagc actgaagata |
| 1441 | ctgctgagta ttcccctttc aagggaggtt acaccaaagg gctagaaggc gagagcctag |
| 1501 | gctgctctgg cagcgctgca gcagggagct ccgggacact tgaactgccg tctaccctgt |
| 1561 | ctctctacaa gtccggagca ctggacgagg cagctgcgta ccagagtcgc gactactaca |
| 1621 | actttccact ggctctggcc ggaccgccgc cccctccgcc gcctccccat ccccacgctc |
| 1681 | gcatcaagct ggagaacccg ctggactacg gcagcgcctg ggcggctgcg gcggcgcagt |
| 1741 | gccgctatgg ggacctggcg agcctgcatg gcgcgggtgc agcgggaccc ggttctgggt |
| 1801 | caccctcagc cgccgcttcc tcatcctggc acactctctt cacagccgaa gaaggccagt |
| 1861 | tgtatggacc gtgtggtggt ggtgggggtg gtggcggcgg cggcggcggc ggcggcggcg |
| 1921 | gcggcggcgg cggcggcggc ggcgaggcgg gagctgtagc cccctacggc tacactcggc |
| 1981 | cccctcaggg gctggcgggc caggaaagcg acttcaccgc acctgatgtg tggtaccctg |
| 2041 | gcggcatggt gagcagagtg ccctatccca gtcccacttg tgtcaaaagc gaaatgggcc |
| 2101 | cctggatgga tagctactcc ggaccttacg gggacatgcg tttggagact gccagggacc |
| 2161 | atgttttgcc cattgactat tactttccac cccagaagac ctgcctgatc tgtggagatg |
| 2221 | aagcttctgg gtgtcactat ggagctctca catgtggaag ctgcaaggtc ttcttcaaaa |
| 2281 | gagccgctga agggaaacag aagtacctgt gcgccagcag aaatgattgc actattgata |
| 2341 | aattccgaag gaaaaattgt ccatcttgtc gtcttcggaa atgttatgaa gcagggatga |
| 2401 | ctctgggagc agctgttgtt gtttctgaaa gaatcttgag ggtgtttgga gtctcagaat |
| 2461 | ggcttcctta aagactacct tcagactctc agctgctcat ccacaacaga gatcagcctt |
| 2521 | tctttgtaga tgattcattc ctggctgcat ttgaaaacca catattgtta attgcttgac |
| 2581 | gaatttaaat cccttgacta cttttcattt caaaaaaaaa aaaaaaa |

SEQ ID NO: 56 Human AR Isoform 5 Amino Acid Sequence (NP_001334993.1)

| | |
|---|---|
| 1 | mevqlglgrv yprppsktyr gafqnlfqsv reviqnpgpr hpeaasaapp gasllllqqq |
| 61 | qqqqqqqqqq qqqqqqqqqq etsprqqqqq qgedgspqah rrgptgylvl deeqqpsqpq |
| 121 | salechperg cvpepgaava askglpqqlp appdeddsaa pstlsllgpt fpglsscsad |
| 181 | lkdilseast mqllqqqqqe avsegsssgr areasgapts skdnylggts tisdnakelc |
| 241 | kavsvsmglg vealehlspg eqlrgdcmya pllgvppavr ptpcaplaec kgsllddsag |
| 301 | kstedtaeys pfkggytkgl egeslgcsgs aaagssgtle lpstlslyks galdeaaayq |
| 361 | srdyynfpla lagppppppp phpharikle npldygsawa aaaaqcrygd laslhgagaa |
| 421 | gpgsgspsaa assswhtlft aeegqlygpc gggggggggg gggggggggg gggeagavap |
| 481 | ygytrppqgl agqesdftap dvwypgfmvs rvpypsptcv ksemgpwmds ysgpygdmrn |
| 541 | trrkrlwkli irsinscics pretevpvrq qk |

SEQ ID NO: 57 Human AR transcript variant 5 cDNA sequence (NM_001348064.1;
CDS: 525 . . . 2243)

| | |
|---|---|
| 1 | gcggagagaa ccctctgttt tcccccactc tctctccacc tcctcctgcc ttccccaccc |
| 61 | cgagtgcgga gccagagatc aaaagatgaa aaggcagtca ggtcttcagt agccaaaaaa |
| 121 | caaaacaaac aaaaacaaaa aagccgaaat aaaagaaaaa gataataact cagttcttat |
| 181 | ttgcacctac ttcagtggac actgaatttg gaaggtggag gattttgttt ttttctttta |
| 241 | agatctgggc atcttttgaa tctacccttc aagtattaag agacagactg tgagcctagc |
| 301 | agggcagatc ttgtccaccg tgtgtcttct tctgcacgag actttgaggc tgtcagagcg |
| 361 | ctttttgcgt ggttgctccc gcaagtttcc ttctctggag cttcccgcag gtgggcagct |
| 421 | agctgcagcg actaccgcat catcacagcc tgttgaactc ttctgagcaa gagaagggga |
| 481 | ggcggggtaa gggaagtagg tggaagattc agccaagctc aaggatggaa gtgcagttag |
| 541 | ggctgggaag ggtctaccct cggccgccgt ccaagaccta ccgaggagct ttccagaatc |
| 601 | tgttccagag cgtgcgcgaa gtgatccaga acccgggccc caggcaccca gaggccgcga |
| 661 | gcgcagcacc tcccggcgcc agtttgctgc tgctgcagca gcagcagcag cagcagcagc |
| 721 | agcagcagca gcagcagcag cagcagcagc agcagcagca gcaagagact agccccaggc |
| 781 | agcagcagca gcagcagggt gaggatggtt ctccccaagc ccatcgtaga ggccccacag |
| 841 | gctacctggt cctggatgag gaacagcaac cttcacagcc gcagtcggcc ctggagtgcc |
| 901 | accccgagag aggttgcgtc ccagagcctg gagccgccgt ggccgccagc aaggggctgc |
| 961 | cgcagcagct gccagcacct ccggacgagg atgactcagc tgccccatcc acgttgtccc |
| 1021 | tgctgggccc cactttcccc ggcttaagca gctgctccgc tgaccttaaa gacatcctga |
| 1081 | gcgaggccag caccatgcaa ctccttcagc aacagcagca ggaagcagta tccgaaggca |
| 1141 | gcagcagcgg gagagcgagg gaggcctcgg gggctcccac ttcctccaag gacaattact |
| 1201 | tagggggcac ttcgaccatt tctgacaacg ccaaggagtt gtgtaaggca gtgtcggtgt |
| 1261 | ccatgggcct gggtgtggag gcgttggagc atctgagtcc aggggaacag cttcgggggg |
| 1321 | attgcatgta cgccccactt ttgggagttc cacccgctgt gcgtcccact ccttgtgccc |
| 1381 | cattggccga atgcaaaggt tctctgctag acgacagcgc aggcaagagc actgaagata |
| 1441 | ctgctgagta ttcccctttc aagggaggtt acaccaaagg gctagaaggc gagagcctag |
| 1501 | gctgctctgg cagcgctgca gcagggagct ccgggacact tgaactgccg tctaccctgt |
| 1561 | ctctctacaa gtccggagca ctggacgagg cagctgcgta ccagagtcgc gactactaca |
| 1621 | actttccact ggctctggcc ggaccgccgc cccctccgcc gcctccccat ccccacgctc |
| 1681 | gcatcaagct ggagaacccg ctggactacg gcagcgcctg ggcggctgcg gcggcgcagt |
| 1741 | gccgctatgg ggacctggcg agcctgcatg gcgcgggtgc agcgggaccc ggttctgggt |
| 1801 | caccctcagc cgccgcttcc tcatcctggc acactctctt cacagccgaa gaaggccagt |
| 1861 | tgtatggacc gtgtggtggt ggtgggggtg gtggcggcgg cggcggcggc ggcggcggcg |
| 1921 | gcggcggcgg cggcggcggc ggcgaggcgg gagctgtagc cccctacggc tacactcggc |
| 1981 | cccctcaggg gctggcgggc caggaaagcg acttcaccgc acctgatgtg tggtaccctg |
| 2041 | gcggcatggt gagcagagtg ccctatccca gtcccacttg tgtcaaaagc gaaatgggcc |
| 2101 | cctggatgga tagctactcc ggaccttacg gggacatgcg aaatacccga agaaagagac |
| 2161 | tctggaaact cattatcagg tctatcaact cttgtatttg ttctcccagg gaaacagaag |
| 2221 | tacctgtgcg ccagcagaaa tgattgcact attgataaat tccgaaggaa aaattgtcca |
| 2281 | tcttgtcgtc ttcggaaatg ttatgaagca gggatgactc tgggagaaaa attccgggtt |
| 2341 | ggcaattgca agcatctcaa aatgaccaga ccctgaagaa aggctgactt gcctcattca |
| 2401 | aaatgagggc tctagagggc tctagtggat agtctggaga aacctggcgt ctgaggctta |
| 2461 | ggagcttagg tttttgctcc tcaacacaga ctttgacgtt ggggttgggg gctactctct |
| 2521 | tgattgctga ctccctccag cgggaccaat agtgttttcc tacctcacag ggatgttgtg |
| 2581 | aggacgggct gtagaagtaa tagtggttac cattcatgta gttgtgagta tcatgattat |
| 2641 | tgtttcctgt aatgtggctt ggcattggca aagtgctttt tgattgttct tgatcacata |
| 2701 | tgatgggggc caggcactga ctcaggcgga tgcagtgaag ctctggctca gtcgcttgct |
| 2761 | tttcgtggtg tgctgccagg aagaaacttt gctgatggga ctcaaggtgt caccttggac |
| 2821 | aagaagcaac tgtgtctgtc tgaggttcct gtggccatct ttatttgtgt attaggcaat |
| 2881 | tcgtatttcc cccttaggtt ctagccttct ggatcccagc cagtgaccta gatcttagcc |
| 2941 | tcaggccctg tcactgagct gaaggtagta gctgatccac agaagttcag taaacaagga |
| 3001 | ccagatttct gcttctccag gagaagaagc cagccaaccc ctctcttcaa acacactgag |
| 3061 | agactacagt ccgactttcc ctcttacatc tagccttact gtagccacac tccttgattg |
| 3121 | ctctctcaca tcacatgctt ctcttcatca gttgtaagcc tctcattctt ctcccaagcc |
| 3181 | agactcaaat attgtattga tgtcaaagaa gaatcactta gagtttggaa tatcttgttc |
| 3241 | tctctctgct ccatagcttc catattgaca ccagtttctt tctagtggag aagtggagtc |
| 3301 | tgtgaagcca gggaaacaca catgtgagag tcagaaggac tctccctgac ttgcctgggg |
| 3361 | cctgtctttc ccaccttctc cagtctgtct aaacacacac acacacacac acacacacac |
| 3421 | acacacgctc tctctctctc tcccccccca acacacacac actctctctc tctctcacac |
| 3481 | acacacacat acacacacac ttctttctct ttcccctgac tcagcaacat tctggagaaa |
| 3541 | agccaaggaa ggacttcagg aggggagttt cccccttctc agggcagaat tttaatctcc |
| 3601 | agaccaacaa gaagttccct aatgtggatt gaaaggctaa tgaggtttat ttttaactac |
| 3661 | tttctatttg tttgaatgtt gcatatttct actagtgaaa ttttccctta ataaagccat |
| 3721 | taatacacc |

SEQ ID NO: 58 Mouse AR Amino Acid Sequence (NP_038504.1)

| | |
|---|---|
| 1 | mevqlglgrv yprppsktyr gafqnlfqsv reaiqnpgpr hpeaaniapp gaclqqrqet |
| 61 | sprrrrrqqh tedgspqahi rgptgylale eeqqpsqqqa aseghpessc lpepgaatap |
| 121 | gkglpqqppa ppdqddsaap stlsllgptf pglsscsadi kdilneagtm qllqqqqqqq |
| 181 | ghqqghqghq qqqevisegs sarareatga pssskdsylg gnstisdsak elckavsvsm |
| 241 | glgvealehl spgeqlrgdc myasllggpp avrptpcapl peckglplde gpgksteeta |
| 301 | eyssfkggya kglegesigc sgsseagssg tleipsslsl yksgaldeaa ayqnrdyynf |
| 361 | plalsgpphp pppthphari klenpldygs awaaaaaqcr ygdlgslhgg svagpstgsp |
| 421 | pattssswht lftaeegqly gpgggggsss psdagpvapy gytrppqglt sqesdysase |
| 481 | vwypggvvnr vpypspncvk semgpwmeny sgpygdmrld strdhvlpid yyfppqktcl |
| 541 | icgdeasgch ygaltcgsck vffkraaegk qkylcasrnd ctidkfrrkn cpscrlrkcy |
| 601 | eagmtlgark lkklgnlklq eegensnags ptedpsqkmt vshiegyecq piflnvleai |
| 661 | epgvvcaghd nnqpdsfaal lsslnelger qlvhvvkwak alpgfrnlhv ddqmaviqys |
| 721 | wmglmvfamg wrsftnvnsr mlyfapdivf neyrmhksrm ysqcvrmrhl sqefgwlqit |
| 781 | pqeflcmkal llfsiipvdg lknqkffdel rmnyikeldr iiackrknpt scsrrfyqlt |
| 841 | klldsvqpia relhqftfdl likshmvsvd fpemmaeiis vqvpkilsgk vkpiyfhtq |

SEQ ID NO: 59 Mouse AR transcript cDNA sequence (NM_013476.4;
CDS: 1026 . . . 3725)

| | |
|---|---|
| 1 | cagcgccccc tcggagatcc ctaggagcca gcctgctggg agaaccagag ggtccggagc |
| 61 | aaacctggag gctgagaggg catcagaggg gaaaagactg agctagccac tccagtgcca |
| 121 | tacagaagct taagggacgc accacgccag ccccagccca gcgacagcca acgcctgttg |
| 181 | cagagcggcg gcttcgaagc cgccgcccag gagctgccct ttcctcttcg gtgaagtttc |
| 241 | taaaagctgc gggagactca gaggaagcaa ggaaagtgtc cggtaggact acggctgcct |
| 301 | ttgtcctctt cccctctacc cttaccccct cctgggtccc ctctccagga gctgactagg |
| 361 | caggctttct ggccaaccct ctcccctaca cccccagctc tgccagccag tttgcacaga |
| 421 | ggtaaactcc ctttggctga gagtagggga gcttgttgca cattgcaagg aaggcttttg |
| 481 | ggagcccaga gactgaggag caacagcacg cccaggagag tccctggttc caggttctcg |
| 541 | cccctgcacc tcctcctgcc cgcccctcac cctgtgtgtg gtgttagaaa tgaaaagatg |
| 601 | aaaaggcagc tagggtttca gtagtcgaaa gcaaaacaaa agctaaaaga aaacaaaaag |
| 661 | aaaatagccc agttcttatt tgcacctgct tcagtggact ttgaatttgg aaggcagagg |
| 721 | atttcccctt ttccctcccg tcaaggtttg agcatctttt aatctgttct tcaagtattt |
| 781 | agagacaaac tgtgtaagta gcagggcaga tcctgtcttg cgcgtgcctt cctttactgg |
| 841 | agactttgag gttatctggg cactcccccc acccaccccc cctcctgcaa gttttcttcc |
| 901 | ccggagcttc ccgcaggtgg gcagctagct gcagatacta catcatcagt caggagaact |
| 961 | cttcagagca agagacgagg aggcaggata agggaattcg gtggaagcta cagacaagct |
| 1021 | caaggatgga ggtgcagtta gggctgggaa gggtctaccc acggccccca tccaagacct |
| 1081 | atcgaggagc gttccagaat ctgttccaga gcgtgcgcga agcgatccag aacccgggcc |
| 1141 | ccaggcaccc tgaggccgct aacatagcac ctcccggcgc ctgtttacag cagaggcagg |
| 1201 | agactagccc ccggcggcgg cggcggcagc agcacactga ggatggttct cctcaagccc |
| 1261 | acatcagagg ccccacaggc tacctggccc tggaggagga acagcagcct tcacagcagc |
| 1321 | aggcagcctc cqaqqqccac cctgagagca gctgcctccc cgagcctggg qcqqccaccq |
| 1381 | ctcctggcaa ggggctgccg cagcagccac cagctcctcc agatcaggat gactcagctg |
| 1441 | ccccatccac gttgtccctg ctgggcccca ctttcccagg cttaagcagc tgctccgccg |
| 1501 | acattaaaga cattttgaac gaggccggca ccatgcaact tcttcagcag cagcaacaac |
| 1561 | agcagcagca ccaacagcag caccaacagc accaacagca gcaggaggta atctccgaag |
| 1621 | gcagcagcgc aagagccagg gaggccacgg gggctccctc ttcctccaag gatagttacc |
| 1681 | tagggggcaa ttcaaccata tctgacagtg ccaaggagtt gtgtaaagca gtgtctgtgt |
| 1741 | ccatgggatt gggtgtggaa gcattggaac atctgagtcc aggggaacag cttcggggag |
| 1801 | actgcatgta cgcgtcgctc ctgggaggtc cacccgcggt gcgtcccact ccttgtgcgc |
| 1861 | cgctgcccga atgcaaaggt cttcccctgg acgaaggccc aggcaaaagc actgaagaga |
| 1921 | ctgctgagta ttcctctttc aagggaggtt acgccaaagg attggaaggt gagagcttgg |
| 1981 | ggtgctctgg cagcagtgaa gcaggtagct ctgggacact tgagatcccg tcctctctgt |
| 2041 | ctctgtataa atctggagca ctagacgagg cagcagcata ccagaatcgc gactactaca |
| 2101 | actttccgct ggctctgtcc gggccgccgc accccccgcc ccctacccat ccacacgccc |
| 2161 | gtatcaagct ggagaaccca ttggactacg gcagcgcctg ggctgcggcg gcagcgcaat |
| 2221 | gccgctatgg ggacttgggt agtctacatg gagggagtgt agccgggccc agcactggat |
| 2281 | cgcccccagc caccacctct tcttcctggc atactctctt cacagctgaa gaaggccaat |
| 2341 | tatatgggcc aggaggcggg ggcggcagca gcagcccaag cgatgccggg cctgtagccc |
| 2401 | cctatggcta cactcggccc cctcaggggc tgacaagcca ggagagtgac tactctgcct |
| 2461 | ccgaagtgtg gtatcctggt ggagttgtga acagagtacc ctatcccagt cccaattgtg |
| 2521 | tcaaaagtga aatgggacct tggatggaga actactccgg accttatggg gacatgcgtt |
| 2581 | tggacagtac cagggaccat gttttaccca tcgactatta ctttccaccc cagaagacct |
| 2641 | gcctgatctg tggagatgaa gcttctggct gtcactacgg agctctcact tgtggcagct |
| 2701 | gcaaggtctt cttcaaaaga gccgctgaag ggaaacagaa gtatctatgt gccagcagaa |
| 2761 | acgattgtac cattgataaa tttcggagga aaaattgccc atcttgtcgt ctccggaaat |
| 2821 | gttatgaagc agggatgact ctgggagctc gtaagctgaa gaaacttgga aatctaaaac |
| 2881 | tacaggagga aggagaaaac tccaatgctg gcagccccac tgaggaccca tcccagaaga |
| 2941 | tgactgtatc acacattgaa ggctatgaat gtcagcctat ctttcttaac gtcctggaag |
| 3001 | ccattgagcc aggagtggtg tgtgccggac atgacaacaa ccaaccagat tcctttgctg |
| 3061 | ccttgttatc tagcctcaat gagcttggag agaggcagct tgtgcatgtg gtcaagtggg |
| 3121 | ccaaggcctt gcctggcttc cgcaacttgc atgtggatga ccagatggcg gtcattcagt |
| 3181 | attcctggat gggactgatg gtatttgcca tgggttggcg gtccttcact aatgtcaact |
| 3241 | ccaggatgct ctactttgca cctgacttgg ttttcaatga gtaccgcatg cacaagtctc |
| 3301 | ggatgtacag ccagtgtgtg aggatgaggc acctgtctca agagtttgga tggctccaaa |
| 3361 | taacccccca ggaattcctg tgcatgaaag cactgctgct cttcagcatt attccagtgg |
| 3421 | atgggctgaa aaatcaaaaa ttctttgatg aacttcgaat gaactacatc aaggaactcg |
| 3481 | atcgcatcat tgcatgcaaa agaaagaatc ccacatcctg ctcaaggcgc ttctaccagc |
| 3541 | tcaccaagct cctggattct gtgcagccta ttgcaagaga gctgcatcag ttcacttttg |
| 3601 | acctgctaat caagtcccat atggtgagcg tggactttcc tgaaatgatg gcagagatca |
| 3661 | tctctgtgca agtgcccaag atcctttctg ggaaagtcaa gcccatctat ttccacacac |
| 3721 | agtgaagatt tggaaaccct aatacccaaa acccaccttg ttccctttcc agatgtcttc |
| 3781 | tgcctgttat ataactctgc actacttctc tgcagtgcct tgggggaaat tcctctactg |
| 3841 | atgtacagtc tgtcgtgaac aggttcctca gttctatttc ctgggcttct ccttcttttt |
| 3901 | ttttcttctt ccctccctct ttcaccctcc catggcacat tttgaatctg ctgcgtattg |
| 3961 | tggctcctgc ctttgttttg atttctgttg tatttctttg aatctgtgat gatcctcttg |
| 4021 | tggcccagtg tcaattgtgc ttgtttatag cactgtgctg tgtgccaacc aagcaaatgt |
| 4081 | ttactcacct tatgccatgg caaatttaga gagctataag tatctggaga agaaacaaac |
| 4141 | agagagaata aaaagcaaaa acaaaaccaa aaaataaaaa aaacacaaac aaaaaacaaa |
| 4201 | accaacaaac aaaacatgct aggtttgttt cttcgtggta tacaaataaa cacataggat |
| 4261 | tcccaaagaa gccgacagtg actagaagaa agtaaaaaat tacaaatcca cgaggagtca |
| 4321 | ctgtttttgt tcatcctgtt tctctgtggg aaacttcagt tgttgttaat ggctattgcc |
| 4381 | attaaagagc aggttgaccc caaagcttta ctgatagggt agagagaaaa gaggacaagg |
| 4441 | agggcagatg gataaccatt acctccccac agcctttgtc cctgagtcct agagtgctca |
| 4501 | gttgcagtgt agttccttgt actgaaatgt gcttcttgtt tgaaaacttg tctgcatgtg |
| 4561 | aatgcctctt ccttccaatc cttttctctc ttaacctctg cttccaccct caattgactt |
| 4621 | tcaatagctt ttctcagagc tttgtactat atgctctctt tagccaaaac ttggccactt |
| 4681 | tcactgaagt tatgtcagtg agaagaaagt ggaaaggtct gactctttgg aaggctctat |
| 4741 | tcagatttat gttcatattt ccatgtgtga gccatagcgg agctttgtga ctggagtcag |
| 4801 | aggaaaagga agtgatggct tagccattct cccattagag atagtgaatg atgatgccat |
| 4861 | agtgcaatca tcctttcctc tgcttttaaa ggacctagag accccatgca gccacattct |
| 4921 | ccctgcacaa gtcttcagtg ttcagtggcc ctgaacttca ccaaaatgca tttaagccaa |
| 4981 | ggtggtaaag cttgtacact tctttggacg tgtttgtaga cactgctaag atctccctct |
| 5041 | caccaccacc acaaaggcta gcaggccagc agccacagca tctatgttta gatgttaata |
| 5101 | gcataaaaga catctcactc aatgtctttc atcaacagta aatttctgga gcccttagaa |
| 5161 | aaattggaaa gaaagcatca aagggaccag acaaaatggg catcttgccc ttgtcctcca |
| 5221 | gagacaatat attcctccca agtggagaaa tgtcaatttc ctcctcagaa caattaaagg |
| 5281 | ggctacccag accatggtgg aagagaaaac taagtaaccc agctgagaaa aatgaagaca |
| 5341 | ctagaaccag aaagcacagg actttttcct ttccatccag catacccatt ggcagaaata |
| 5401 | atggaaggaa aagagaaggc cagaagaaaa tacagactgc tgaagtcttc agaggcaaag |
| 5461 | tctaaagcca gatgaatacc atctggctag atgggcatca gtttgctcat cctcctctat |
| 5521 | tgccattgct gggctgactt tggccaaagt tacttcgaat ctccaccata gttgtcccct |
| 5581 | ctcagtcaga gggtgcagga ccactgaaac attctatcca ccgtgactct cattggacag |
| 5641 | atctggccgg tgtggctaca aatagactgc acccataaac tcagggcaag ccctgggtca |
| 5701 | ctggtttcat gtagtctgtt gacagccttc tttactgtgg actctgttcc tcaaccttga |
| 5761 | gtgcaggagg aatgcacatc tacttttgcc tttgtatcat tcctcctcac tcagctcttc |
| 5821 | acctccctgc agaccttaag aaatcagggg ccagctgcca agctgactct tttggttggt |
| 5881 | actatgttaa ctgaaaaggt gatttccgaa ggacaggttt tcttccctga tttctttgtt |
| 5941 | gctattaata gcaaaaacaa acttgcaaaa caacttcttt aacaaggaag ggaggatata |
| 6001 | tacaatgggt gatatggtaa tccaaccctg cttgacaaaa actgaagctg acaggttaca |
| 6061 | tttaaaaaca aaacaaaaca aaacgggaca gtttctgatt tgctttgtga caacaccatc |
| 6121 | tggcttatgt acaggagctc tcttagctgt tccttaaaca gaaaaaaaat cattactcct |
| 6181 | tttagttaaa tttggttaca ttttaatagt ttctttacat ctattctgaa gcaatttttg |
| 6241 | tcttctgtgg tacatggatt ttattataac attctaatat ttgtctttgt aaatactaga |
| 6301 | gactctttga tccatttctc taggaagttt ttcatcttat ggagttctga atcatgactt |
| 6361 | ttatctttat gaatgtatat gctttttact tgcaaaagcc aaaaagagtg aaacagcagt |
| 6421 | gcaattaaag caacaccaac taaactccaa atttccaagt gacaatatta gagaaaaaca |
| 6481 | gcatacacat ggctttatgc ctactgcttc tgcggtgggg tttgggtgcg caatggaaac |
| 6541 | tgtagcttgg ctgtgttctc ccacacaagt gaagaagaga ttggtttttg cttttttgga |
| 6601 | ttttgtgttt cttttctgtt ttgttttgtt ttgttttgtt ttgctttgct ttctttggcc |
| 6661 | atcaatgttc caactaatat gattggcgga gcacgtgctc tgctcagtag agtgaatgtt |
| 6721 | gctggtgcac tatgctcacc tgtgaacggc tggccatttc tccattcata tggttaagat |
| 6781 | ggaagatgag gatcacttac cagagaagtc aaggtgatca tctccaaaga ggtttacagt |
| 6841 | gcttggtagg aatggaaaat gaggacaaga aaaagaggag aaccatggag aaggcccaac |
| 6901 | tgggcaggac agcagccagc tgccaaagtc acgaactctg ggattcaaga agagtcgtgt |
| 6961 | agtgctttca actctcatcc gcaggcagct cactgtgtgt ggactctgag ctgacacggg |
| 7021 | agttggcttc tttgttccat agattttcta tgccacaggc aatattattg ttcttggaaa |
| 7081 | gttcattatt tttttaaatt accttactct cagaaaggga tttttttgaa ggattctgtc |
| 7141 | atatatcttt ggaaaacaga aaatcagtaa tatgtatatt tttatgtatg ttcactggca |
| 7201 | ctaaaaaaaa aaaaaaaaag aaaagaaaaa aaagagaaaa aaaaaagctt cactctgtcc |
| 7261 | tttgggtagt tgctgaggtt aattgtccag gttgagaaat gtgcttctgc taacatcctt |
| 7321 | ctctgtccac actctatttc taagtacata taggcatata taggaagata tattcaacac |
| 7381 | actttaagaa aaaagtatgt ccaccatcca catgataacc acaatgatac tccacaaatt |
| 7441 | acatgacttt aagcttcaag caacttctaa ctgattcatt catttatagc cttgccctct |
| 7501 | tctttccctt aaatttggcc cagcacaaag acccaagcca cccttatacc tccctaagac |
| 7561 | ttaagccagc accagacttc agaaggtttt ctgaagacaa ctgacttgct atccctgcat |
| 7621 | gaccctagca tggtcctgca aacacaagag actaattata attctcctcc actaattgcc |
| 7681 | tgggtcacag gtcattgggc caaggccatg attcttatgc ttacgaacca ctaatgctaa |
| 7741 | cctactagat taaatcctga actgaaagtt aaaagaagcc atttagcatg tgaaacttct |
| 7801 | tggagtaaga agtttctgtc ccggctgcct ttgcaaacag gtttgctttc accacttatc |
| 7861 | tccttgaaaa tctttgaagg cctttttttt ttaagtagaa aaggagatga aagcattata |
| 7921 | ttatgtaacc aaagattata ttgtatctaa gataccaaat tttttaaggg cagggaagga |
| 7981 | gcaagcatta gtgcctcttt ggtaaattat ccaaagacag actgaaggac ttttctgatg |
| 8041 | attgacttag aagactttgt ggggaggggt tgtctcacaa tatacatatt tagaagtgtt |
| 8101 | gagaataatt tggggggaaa tgggattata gtgtccttca ctaactgatt ttataagcag |
| 8161 | aactagcttt cctttttttt ttttttaaag tagttacaaa gcaaattctt aaagctccat |
| 8221 | ctttgcatgg ttagaaatgg agctggtctt ggccactgtg tttactagtg cccatgttag |
| 8281 | cttatttgaa gatgtgaagc ccttgataag aaggggtaca tttaaaggat tagatttttg |
| 8341 | cactagaagg agggcaggca gaaaccctca tttctgccca gtttggacag cacaaaaagt |
| 8401 | tctctgcagt ttaaggcaga aagttgaaat atattgtaaa tgagtatttg tatccatgtt |
| 8461 | tcaaaactga attctatata tagatgtaat gtgttctgat agctttacct ttctctgcac |
| 8521 | ctttatattt ggttccaggt catatctgat gccatgtact tgtaagagag gttgcagtta |
| 8581 | catttttgga tgctctctca gaatggataa gacacctgga ttgatcagat aactgagatc |
| 8641 | tcttcccttc ttgggcctgg tgttgaggcc ttgcaaaggg gtggaagagg aaagggtagg |
| 8701 | gtacatgatg tattgcactt tactagctta agacggatga atgtggaaag ggtggtgaaa |
| 8761 | tttcattgaa aatgcctagg aattgcaata gggagaaatc cagatgtggg gccaggtgcc |
| 8821 | cacccaaagg actggccagc agcctcttca tgggatctga ggcattggga aaaggaaggc |
| 8881 | tatttccttg gttttcacca tccttgttag agaagggcag ttgcctggtc ttgggaacct |
| 8941 | ggagcaaacg ctccttctgt cacatcaatt ctttcccctg caattgaggt gctcttgcta |
| 9001 | ctgggtgtcc gtgtgctcta attctggttc tggatatgtt ctgtaaagat tttgataatt |
| 9061 | gctaatgtat ttttctctgt taaaaatttg ttagtgtgtt agaagtcata tctctgtagg |
| 9121 | tacagatcct ttgctaccca tgagtagagg gatttttttt cttcaattaa gagtttgacc |
| 9181 | ctggggtctg ttgcccagag cccatccaga aaaaaaaatc cacatttgtc acaatttttc |
| 9241 | tgaaatttca gtcaaggtaa cagatcgctg ggagttctct ttaccccccc aaaaaagcag |
| 9301 | ataattgaat ttagcaggtg gtgttttaga gcaaaaaaca aaacagcctt tgacccagct |
| 9361 | ttaatatgac ccaatttaat ctggccagga agcaggtaat gtgtattaat tggcttccaa |
| 9421 | tcctggttga gtgtagcaag gttctacttt gtttcctagt tccttttgtt acatggcctt |
| 9481 | tcacagaaag gattgactgg gtttgcagta tatcttatgg ccttagcacc tattgctaac |
| 9541 | tgtcctgaag ggaattgcct atggggttgt cctataagcc acttctatca ttaaaagcag |
| 9601 | ccaccaatgg aatctcccag gtttgaaaaa aaaaaaaaaa aacagatggt cctttaccat |
| 9661 | tcattgacac acatccctgc tttcctgtag acagattgac tggacattga ttagggaata |
| 9721 | catggcaaat gacatgctta cactaccctg gagattaatt tggcagtagg agggaataga |
| 9781 | caatgtaacc aagaatgtaa tgtaattctt atagagataa gaattaaatc tggatgtgga |
| 9841 | gagagcaaag agagaaagca ttcaattttt ttttcaaaag aaaccaattt attttgcttg |
| 9901 | aaacttcttt cgctggggct tcagttctca cagcggctct tggtctccac tgggcagcag |
| 9961 | gaccagcccc aagcgctagt gttctgttct ctttttgtaa tcttggaatc ttttgttgct |
| 10021 | ctaaatacaa ttaaaaatgc cagaaacttc tttgttggaa tac |

* Included in Tables 1A and 1B are orthologs of the proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Tables 1A and 1B, or a portion thereof. Such polypeptides can have a function of the full-length polypeptide as described further herein.

TABLE 1C

| | | | Methylation | | | |
|---|---|---|---|---|---|---|
| | | | status | | | Nearest |
| | Start | End | (NE vs | | Distance | Promoter |
| Chr | (hg19) | (hg19) | Adeno) | Annotation | to TSS | ID |
| chr1 | 241206098 | 241206097 | hyper | intron | −89475 | NR_036069 |
| | | | | (NM_001282773, | | |
| | | | | intron 3 of 15) | | |
| chr18 | 47258089 | 47258088 | hyper | Intergenic | 82163 | NM_006111 |
| chr21 | 32546326 | 32546325 | hyper | intron | −135530 | NM_001099219 |
| | | | | (NM_003253, | | |
| | | | | intron 16 of 28) | | |
| chr5 | 1548487 | 1548486 | hyper | Intergenic | −24410 | NM_024830 |
| chr5 | 71146894 | 71146893 | hyper | Intergenic | 131903 | NM_004291 |
| chr5 | 142194123 | 142194122 | hyper | intron | 43830 | NM_015071 |
| | | | | (NM_015071, | | |
| | | | | intron 1 of 22) | | |
| chr7 | 7799018 | 7799017 | hyper | intron | −40779 | NM_002947 |
| | | | | (NM_001302350, | | |
| | | | | intron 3 of 4) | | |
| chr7 | 127841628 | 127841627 | hyper | Intergenic | −6298 | NR_029596 |
| chr8 | 53499560 | 53499559 | hyper | Intergenic | −21538 | NM_207413 |
| chr8 | 73479990 | 73479989 | hyper | exon | 30363 | NM_004770 |
| | | | | (NM_004770, | | |
| | | | | exon 2 of 3) | | |
| chr8 | 73849054 | 73849053 | hyper | exon | −72044 | NM_003218 |
| | | | | (NM_004770, | | |
| | | | | exon 3 of 3) | | |
| chr8 | 140727998 | 140727997 | hyper | Intergenic | −12698 | NR_104210 |
| chr8 | 141994084 | 141994083 | hyper | intron | 17329 | NM_001199649 |
| | | | | (NM_153831, | | |
| | | | | intron 1 of 31) | | |
| chr8 | 142367954 | 142367953 | hyper | exon | 9412 | NM_005293 |
| | | | | (NM_005293, | | |
| | | | | exon 2 of 2) | | |
| chr8 | 143395944 | 143395943 | hyper | intron | 88667 | NM_145003 |
| | | | | (NM_001291931, | | |
| | | | | intron 6 of 10) | | |
| chr9 | 36748433 | 36748432 | hyper | Intergenic | 75164 | NR_039686 |
| chr9 | 38437314 | 38437313 | hyper | Intergenic | −12869 | NM_001007563 |
| chr9 | 93792388 | 93792387 | hyper | Intergenic | 45027 | NR_033912 |
| chr9 | 133769512 | 133769511 | hyper | intron | 2688 | NM_198180 |
| | | | | (NM_198180, | | |
| | | | | intron 2 of 2) | | |
| chr9 | 138719304 | 138719303 | hyper | exon | 79702 | NM_015447 |
| | | | | (NM_015447, | | |
| | | | | exon 8 of 17) | | |

| | Gene | | | Gene |
|---|---|---|---|---|
| Chr | Name | Gene Alias | Gene Description | Type |
| chr1 | MIR3123 | mir-3123 | microRNA 3123 | ncRNA |
| chr18 | ACAA2 | DSAEC | acetyl-CoA | protein- |
| | | | acyltransferase 2 | coding |
| chr21 | KRTAP19-8 | | keratin associated | protein- |
| | | | protein 19-8 | coding |
| chr5 | LPCAT1 | AYTL2\|PFAAP3\|lpcat | lysophosphatidylcholine | protein- |
| | | | acyltransferase 1 | coding |
| chr5 | CARTPT | CART | CART | protein- |
| | | | prepropeptide | coding |
| chr5 | ARHGAP26 | GRAF\|GRAF1\|OPHN1L\| | Rho GTPase | protein- |
| | | OPHN1L1 | activating protein | coding |
| | | | 26 | |
| chr7 | RPA3 | REPA3 | replication protein | protein- |
| | | | A3 | coding |
| chr7 | MIR129-1 | MIR-129b\|MIRN129-1\| | microRNA 129-1 | ncRNA |
| | | mir-129-1 | | |
| chr8 | FAM150A | UNQ9433 | family with | protein- |
| | | | sequence | coding |
| | | | similarity 150, | |
| | | | member A | |
| chr8 | KCNB2 | KV2.2 | potassium channel, | protein- |
| | | | voltage gated Shab | coding |
| | | | related subfamily | |
| | | | B, member 2 | |
| chr8 | TERFI | PIN2\|TRBF1\|TRF\|TRF1\| | telomeric repeat | protein- |
| | | hTRF1-AS\|t-TRF1 | binding factor | coding |
| | | | (NIMA- | |
| | | | interacting) 1 | |
| chr8 | KCNK9 | K2p9.1\|KT3.2\|TASK-3\| | potassium channel, | protein- |
| | | TASK3 | two pore domain | coding |
| | | | subfamily K, | |
| | | | member 9 | |
| chr8 | PTK2 | FADK\|FAK\|FAK1\|FRNK\| | protein tyrosine | protein- |
| | | PPP1R71\|p125FAK\|pp125FAK | kinase 2 | coding |
| chr8 | GPR20 | — | G protein-coupled | protein- |
| | | | receptor 20 | coding |
| chr8 | TSNARE1 | — | t-SNARE domain | protein- |
| | | | containing 1 | coding |
| chr9 | MIR4475 | — | microRNA 4475 | ncRNA |
| chr9 | IGFBPL1 | IGFBP-RP4\|IGFBPRP4\|bA113024.1 | insulin like growth | protein- |
| | | | factor binding | coding |
| | | | protein-like 1 | |
| chr9 | LOC100129316 | — | uncharacterized | ncRNA |
| | | | LOC100129316 | |
| chr9 | QRFP | 26RFa\|P518 | pyroglutamylated | protein- |
| | | | RFamide peptide | coding |
| chr9 | CAMSAP1 | PRO2405\|bA100C15.1 | calmodulin | protein- |
| | | | regulated spectrin- | coding |
| | | | associated protein 1 | |

TABLE 1D

| | | | Methylation | | | |
|---|---|---|---|---|---|---|
| | | | status | | | Nearest |
| | Start | End | (NE vs | | Distance | Promoter |
| Chr | (hg19) | (hg19) | Adeno) | Annotation | to TSS | ID |
| chr1 | 202039153 | 202039152 | hypo | Intergenic | −52877 | NM_004767 |
| chr14 | 100039425 | 100039424 | hypo | intron | 31303 | NM_001144995 |
| | | | | (NM_001144995, | | |
| | | | | intron 1 of 5) | | |
| chr18 | 8660098 | 8660097 | hypo | Intergenic | 47522 | NR_120598 |
| chr18 | 14917016 | 14917015 | hypo | Intergenic | −29251 | NR_104164 |
| chr18 | 25442246 | 25442245 | hypo | Intergenic | 174304 | NM_001308176 |
| chr2 | 89530057 | 89530056 | hypo | Intergenic | 418172 | NR_039635 |
| chr20 | 20421462 | 20421461 | hypo | intron | 72696 | NM_002196 |
| | | | | (NM_020343, | | |
| | | | | intron 37 of 39) | | |
| chr20 | 21563921 | 21563920 | hypo | intron | 13258 | NR_109880 |
| | | | | (NR_109880, | | |
| | | | | intron 1 of 3) | | |
| chr21 | 46885392 | 46885391 | hypo | intron | 9967 | NM_130444 |
| | | | | (NM_130444, | | |
| | | | | intron 1 of 41) | | |
| chr3 | 193490472 | 193490471 | hypo | Intergenic | −145350 | NR_046634 |
| chr5 | 36521979 | 36521978 | hypo | Intergenic | −84479 | NM_001289940 |
| chr5 | 39390555 | 39390554 | hypo | exon | −25899 | NM_001737 |
| | | | | (NM_001343, | | |
| | | | | exon 5 of 15) | | |
| chr5 | 168573593 | 168573592 | hypo | intron | 117106 | NR_030311 |
| | | | | (NM_001271946, | | |
| | | | | intron 4 of 35) | | |
| chr7 | 2006579 | 2006578 | hypo | intron | −26392 | NM_001304525 |
| | | | | (NM_001304523, | | |
| | | | | intron 14 of 17) | | |
| chr7 | 129912398 | 129912397 | hypo | intron | 5694 | NM_001869 |
| | | | | (NM_001869, | | |
| | | | | intron 4 of 10) | | |
| chr8 | 61990390 | 61990389 | hypo | Intergenic | −110082 | NR_034003 |
| chr8 | 69087227 | 69087226 | hypo | intron | −155731 | NM_052958 |
| | | | | (NM_024870, | | |
| | | | | intron 35 of 39) | | |
| chr8 | 133070961 | 133070960 | hypo | intron | 667 | NM_001080399 |
| | | | | (NM_001080399, | | |
| | | | | intron 1 of 13) | | |
| chr8 | 141458926 | 141458925 | hypo | intron | 8936 | NM_001160372 |
| | | | | (NM_031466, | | |
| | | | | intron 2 of 22) | | |
| chr9 | 134913164 | 134913163 | hypo | intron | 42111 | NM_001253881 |
| | | | | (NM_001253881, | | |
| | | | | intron 2 of 6) | | |

| | Gene | | | Gene |
|---|---|---|---|---|
| Chr | Name | Gene Alias | Gene Description | Type |
| chr1 | GPR37L1 | ET(B)R-LP-2\|ETBR-LP-2 | G protein-coupled | protein- |
| | | | receptor 37 like 1 | coding |
| chr14 | CCDC85C | — | coiled-coil domain | protein- |
| | | | containing 85 C | coding |
| chr18 | GACAT2 | MTCL1-AS1\|MTCL1AS1 | gastric cancer associated | ncRNA |
| | | | transcript 2 (non-protein | |
| | | | coding) | |
| chr18 | LINC01443 | — | long intergenic non- | ncRNA |
| | | | protein coding RNA | |
| | | | 1443 | |
| chr18 | CDH2 | CD325\|CDHN\|CDw325\|NCAD | cadherin 2, type 1, N- | protein- |
| | | | cadherin (neuronal) | coding |
| chr2 | MIR4436A | mir-4436a | microRNA 4436a | ncRNA |
| chr20 | INSM1 | IA-1\|IA1 | insulinoma-associated 1 | protein- |
| | | | | coding |
| chr20 | LOCI01929625 | — | uncharacterized | ncRNA |
| | | | LOC101929625 | |
| chr21 | COL18A1 | KNO\|KNO1\|KS | collagen type XVIII, | protein- |
| | | | alpha 1 | coding |
| chr3 | OPA1-AS1 | — | OPA1 antisense RNA 1 | ncRNA |
| chr5 | SLC1A3 | EA6\|EAAT1\|GLAST\|GLAST1 | solute carrier family 1 | protein- |
| | | | (glial high affinity | coding |
| | | | glutamate transporter), | |
| | | | member 3 | |
| chr5 | C9 | ARMD15\|C9D | complement component | protein- |
| | | | 9 | coding |
| chr5 | MIR585 | MIRN585\|hsa-mir-585\|mir-585 | microRNA 585 | ncRNA |
| chr7 | MAD1L1 | MAD1\|PIG9\|TP53I9\|TXBP181 | MAD1 mitotic arrest | protein- |
| | | | deficient-like 1 (yeast) | coding |
| chr7 | CPA2 | | carboxypeptidase A2 | protein- |
| | | | (pancreatic) | coding |
| chr8 | LOC100130298 | | hCG1816373-like | ncRNA |
| chr8 | C8orf34 | VEST-1\|VEST1 | chromosome 8 open | protein- |
| | | | reading frame 34 | coding |
| chr8 | OC90 | PLA2L | otoconin 90 | protein- |
| | | | | coding |
| chr8 | TRAPPC9 | IBP\|IKBKBBP\|MRT13\|NIBP\| | trafficking protein | protein- |
| | | T1\|TRS120 | particle complex 9 | coding |
| chr9 | MED27 | CRAP34\|CRSP34\|CRSP8\|TRAP37 | mediator complex | protein- |
| | | | subunit 27 | coding |

### II. Subjects

In one embodiment, the subject for whom predicted likelihood of having castration-resistant neuroendocrine prosate cancer (CRPC-NE) or at risk for developing CRPC-NE is determined, is a mammal (e.g., mouse, rat, primate, non-human mammal, domestic animal, such as a dog, cat, cow, horse, and the like), and is preferably a human. In another embodiment, the subject is an animal model of prostate cancer. For example, the animal model can be an orthotopic xenograft animal model of a human-derived prostate cancer.

In another embodiment of the methods of the present invention, the subject is afflicted with castration-resistant prostate cancer (CRPC). In still another embodiment, the subject is resistant to an androgen receptor (AR)-directed therapy.

In another embodiment of the methods of the present invention, the subject has not undergone treatment, such as chemotherapy, radiation therapy, targeted therapy, and/or immunotherapies. In still another embodiment, the subject has undergone treatment, such as chemotherapy, radiation therapy, targeted therapy, and/or immunotherapies.

In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient.

### III. Sample Collection, Preparation and Separation

In some embodiments, genomic (e.g., deletion, gain, or mutation) and/or epigenomic (e.g., hypermethylation or hypomethylation) alterations of the biomarkers in a sample of interest, such as from a subject, is compared to a control (standard) sample. The sample from the subject is typically from a diseased tissue, such as cancer cells or tissues. In certain embodiments, the sample is cell-free DNA (cfDNA) or circulating tumor DNA (ctDNA). Reagents and protocols for obtaining and analyzing cfDNA and ctDNA, such as circulating in the blood stream or other tissue, are commercially available as described in the Examples and also well-known in the art (see, for example, Anker et al. (1999) Cancer and Metastasis Rev. 18:65-73; Wua et al. (2002) Clin. Chim. Acta 321:77-87; Fiegl et al. (2005) Cancer Res. 15:1141; Pathak et al. (2006) Clin. Chem. 52:1833-1842; Schwarzenbach et al. (2009) Clin. Cancer Res. 15:1032; Schwarzenbach et al. (2011) Nat. Rev. Cancer 11:426-437).

The control sample can be from the same subject or from a different subject. The control sample is typically a normal, non-diseased sample. However, in some embodiments, such as for staging of disease or for evaluating the efficacy of treatment, the control sample can be from a diseased tissue (e.g., from a subject with castration resistant prostate adenocarcinoma (CRPC-Adeno)). The control sample can be a combination of samples from several different subjects. In some embodiments, the genomic and/or epigenomic alterations of the biomarkers from a subject is compared to a pre-determined level. This pre-determined level is typically obtained from normal samples. As described herein, a “pre-determined” biomarker genomic and/or epigenomic alteration(s) may be a biomarker genomic and/or epigenomic alteration(s) used to, by way of example only, diagnose the subject (e.g., based on the present or absence of specific genomic and/or epigenomic alteration(s) of the biomarkers, evaluate whether a subject is afflicted with castration-resistant neuroendocrine prosate cancer (CRPC-NE) or at risk for developing CRPC-NE). A pre-determined biomarker genomic and/or epigenomic alteration(s) may be determined in populations of patients with or without cancer. The pre-determined biomarker genomic and/or epigenomic alteration(s) can be a single number, equally applicable to every patient, or the pre-determined biomarker genomic and/or epigenomic alteration(s) can vary according to specific subpopulations of patients. Age, weight, height, and other factors of a subject may affect the pre-determined biomarker genomic and/or epigenomic alteration(s) of the individual. Furthermore, the pre-determined biomarker genomic and/or epigenomic alteration(s) can be determined for each subject individually. In one embodiment, the genomic and/or epigenomic alteration(s) determined and/or compared in a method described herein are based on absolute measurements.

In another embodiment, the amounts determined and/or compared in a method described herein are based on relative measurements, such as ratios (e.g., biomarker copy numbers, methylation level, and/or mutations before a treatment vs. after a treatment, such biomarker measurements relative to a spiked or man-made control, such biomarker measurements relative to the expression of a housekeeping gene, and the like). For example, the relative analysis can be based on the ratio of pre-treatment biomarker measurement as compared to post-treatment biomarker measurement. Pre-treatment biomarker measurement can be made at any time prior to initiation of anti-cancer therapy. Post-treatment biomarker measurement can be made at any time after initiation of anti-cancer therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of anti-cancer therapy, and even longer toward indefinitely for continued monitoring. Treatment can comprise anti-cancer therapy, such as a platum-based chemotherapy alone or in combination with other anti-cancer agents, such as with AR-targeted therapies and/or immunotherapies.

The pre-determined biomarker genomic and/or epigenomic alteration(s) can be any suitable standard. For example, the pre-determined biomarker genomic and/or epigenomic alteration(s) can be obtained from the same or a different human for whom a patient selection is being assessed. In one embodiment, the pre-determined biomarker genomic and/or epigenomic alteration(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments of the present invention the change of biomarker genomic and/or epigenomic alteration(s) from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cutoff values apply equally when the measurement is based on relative changes, such as based on the ratio of pre-treatment biomarker measurement as compared to post-treatment biomarker measurement.

Biological samples can be collected from a variety of sources from a patient including a body fluid sample, cell sample, or a tissue sample comprising nucleic acids and/or proteins. “Body fluids” refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In a preferred embodiment, the subject and/or control sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow. In one embodiment, the sample is serum, plasma, or urine. In another embodiment, the sample is serum.

The samples can be collected from individuals repeatedly over a longitudinal period of time (e.g., once or more on the order of days, weeks, months, annually, biannually, etc.). Obtaining numerous samples from an individual over a period of time can be used to verify results from earlier detections and/or to identify an alteration in biological pattern as a result of, for example, disease progression, drug treatment, etc. For example, subject samples can be taken and monitored every month, every two months, or combinations of one, two, or three month intervals according to the present invention. In addition, the biomarker amount and/or activity measurements of the subject obtained over time can be conveniently compared with each other, as well as with those of normal controls during the monitoring period, thereby providing the subject's own values, as an internal, or personal, control for long-term monitoring.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g., aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermable membrane in a process in which ions are transported through semi-permeable membranes from one solution to another under the influence of a potential gradient. Since the membranes used in electrodialysis may have the ability to selectively transport ions having positive or negative charge, reject ions of the opposite charge, or to allow species to migrate through a semipermable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC), etc.

In some embodiments, whole blood is collected from the subject and plasma layer is separated by centrifugation. Cell free DNA may be then extracted from the plasma using methods known in the art. The isolated cell free DNA can be used to determine the genomic and/or epigenimic alterations of the biomarkers provide herein.

### IV. Analyzing Genomic and/or Epigenomic Alterations of Biomarker

Genomic and/or epigenomic alterations of biomarker can be analyzed according to the methods described herein and techniques known to the skilled artisan.

a. Methods for Detection of Copy Number

Methods of evaluating the copy number of a biomarker nucleic acid are well-known to those of skill in the art. The presence or absence of chromosomal gain or loss can be evaluated simply by a determination of copy number of the regions or markers identified herein.

In one embodiment, a biological sample is tested for the presence of copy number changes in genomic loci containing the biomarkers in Table 1A or 1B.

Methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In one embodiment, evaluating the biomarker gene copy number in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well-known in the art to detect RNA can be used, such that higher or lower expression relative to an appropriate control (e.g., a non-amplified portion of the same or related cell tissue, organ, etc.) provides an estimate of the relative copy number of the target nucleic acid.

An alternative means for determining genomic copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained. The probes are typically labeled, e.g., with radioisotopes or fluorescent reporters. In one embodiment, probes are sufficiently long so as to specifically hybridize with the target nucleic acid(s) under stringent conditions. Probes generally range in length from about 200 bases to about 1000 bases. In some applications it is necessary to block the hybridization capacity of repetitive sequences. Thus, in some embodiments, tRNA, human genomic DNA, or Cot-I DNA is used to block non-specific hybridization.

An alternative means for determining genomic copy number is comparative genomic hybridization. In general, genomic DNA is isolated from normal reference cells, as well as from test cells (e.g., tumor cells) and amplified, if necessary. The two nucleic acids are differentially labeled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labeled DNA sequences are then rendered in a visualizable form, if necessary. Chromosomal regions in the test cells which are at increased or decreased copy number can be identified by detecting regions where the ratio of signal from the two DNAs is altered. For example, those regions that have decreased in copy number in the test cells will show relatively lower signal from the test DNA than the reference compared to other regions of the genome. Regions that have been increased in copy number in the test cells will show relatively higher signal from the test DNA. Where there are chromosomal deletions or multiplications, differences in the ratio of the signals from the two labels will be detected and the ratio will provide a measure of the copy number. In another embodiment of CGH, array CGH (aCGH), the immobilized chromosome element is replaced with a collection of solid support bound target nucleic acids on an array, allowing for a large or complete percentage of the genome to be represented in the collection of solid support bound targets. Target nucleic acids may comprise cDNAs, genomic DNAs, oligonucleotides (e.g., to detect single nucleotide polymorphisms) and the like. Array-based CGH may also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays. Methods of preparing immobilized chromosomes or arrays and performing comparative genomic hybridization are well-known in the art (see, e.g., U.S. Pat. Nos. 6,335,167; 6,197,501; 5,830,645; and 5,665,549 and Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), etc.) In another embodiment, the hybridization protocol of Pinkel, et al. (1998) Nature Genetics 20: 207-211, or of Kallioniemi (1992) Proc. Natl Acad Sci USA 89:5321-5325 (1992) is used.

In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well-known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the present invention. In fluorogenic quantitative PCR, quantitation is based on amount of fluorescence signals, e.g., TaqMan and SYBR green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

Loss of heterozygosity (LOH) and major copy proportion (MCP) mapping (Wang, Z. C., et al. (2004) Cancer Res 64(1):64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93; Li et al., (2008) MBC Bioinform. 9, 204-219) may also be used to identify regions of amplification or deletion.

b. Methods for Detection of Biomarker Nucleic Acid Expression

Biomarker expression may be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g. mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In another embodiment, detecting or determining expression levels of a biomarker and functionally similar homologs thereof, including a fragment or genetic alteration thereof (e.g., in regulatory or promoter regions thereof) comprises detecting or determining RNA levels for the marker of interest. In one embodiment, one or more cells from the subject to be tested are obtained and RNA is isolated from the cells. In a preferred embodiment, a sample of breast tissue cells is obtained from the subject.

In one embodiment, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278: 1481; Emmert-Buck et al. (1996) Science 274:998; Fend et al. (1999) Am. J. Path. 154: 61 and Murakami et al. (2000) Kidney Int. 58:1346). For example, Murakami et al., supra, describe isolation of a cell from a previously immunostained tissue section.

It is also be possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from the tissue sample by a variety of methods, e.g., the guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin.

The RNA sample can then be enriched in particular species. In one embodiment, poly(A)+RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In a preferred embodiment, the RNA population is enriched in marker sequences. Enrichment can be undertaken, e.g., by primer-specific cDNA synthesis, or multiple rounds of linear amplification based on cDNA synthesis and template-directed in vitro transcription (see, e.g., Wang et al. (1989) Proc. Natl. Acad. Sci. U.S.A. 86: 9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e.g., Kwoh et al., Proc. Natl. Acad. Sci. USA 86, 1173 (1989)).

Many techniques are known in the state of the art for determining absolute and relative levels of gene expression, commonly used techniques suitable for use in the present invention include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising biomarker DNA. Positive hybridization signal is obtained with the sample containing biomarker transcripts. Methods of preparing DNA arrays and their use are well-known in the art (see, e.g., U.S. Pat. Nos: 6,618,6796; 6,379,897; 6,664,377; 6,451,536; 548,257; U.S. 20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescently-labeled cDNA probes are generated. The microarrays capable of hybridizing to marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

In one embodiment, the biological sample contains polypeptide molecules from the test subject. Alternatively, the biological sample can contain mRNA molecules from the test subject or genomic DNA molecules from the test subject.

In another embodiment, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

c. Methods for Detection of Biomarker Protein Expression The activity or level of a biomarker protein can be detected and/or quantified by detecting or quantifying the expressed polypeptide. The polypeptide can be detected and quantified by any of a number of means well-known to those of skill in the art. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

For example, ELISA and RIA procedures may be conducted such that a desired biomarker protein standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In one embodiment, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Enzymatic and radiolabeling of biomarker protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect biomarker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy.

Anti-biomarker protein antibodies, such as intrabodies, may also be used for imaging purposes, for example, to detect the presence of biomarker protein in cells and tissues of a subject. Suitable labels include radioisotopes, iodine (¹²⁵I, ¹²¹I) carbon (¹⁴C), sulphur (³⁵S), tritium (³H), indium (¹¹²In), and technetium (⁹⁹mTc), fluorescent labels, such as fluorescein and rhodamine, and biotin.

For in vivo imaging purposes, antibodies are not detectable, as such, from outside the body, and so must be labeled, or otherwise modified, to permit detection. Markers for this purpose may be any that do not substantially interfere with the antibody binding, but which allow external detection. Suitable markers may include those that may be detected by X-radiography, NMR or MM. For X-radiographic techniques, suitable markers include any radioisotope that emits detectable radiation but that is not overtly harmful to the subject, such as barium or cesium, for example. Suitable markers for NMR and MM generally include those with a detectable characteristic spin, such as deuterium, which may be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99. The labeled antibody or antibody fragment will then preferentially accumulate at the location of cells which contain biomarker protein. The labeled antibody or antibody fragment can then be detected using known techniques.

Antibodies that may be used to detect biomarker protein include any antibody, whether natural or synthetic, full length or a fragment thereof, monoclonal or polyclonal, that binds sufficiently strongly and specifically to the biomarker protein to be detected. An antibody may have a K_{d} of at most about 10⁻⁶M, 10⁻⁷M, 10⁻⁸M, 10⁻⁹M, 10⁻¹⁰ M, 10⁻¹¹ M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins.

Antibodies are commercially available or may be prepared according to methods known in the art.

Antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies. For example, antibody fragments capable of binding to a biomarker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies. Antibodies produced from a library, e.g., phage display library, may also be used.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

d. Methods for Detection of Biomarker Structural Alterations

The following illustrative methods can be used to identify the presence of a structural alteration (e.g., genomic muations) in a biomarker (e.g., biomarkers in Tables 1A and 1B) in order to, for example, assess whether a subject is affliacted with CRPC-NE or at the risk of developing CRPC-NE.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a biomarker nucleic acid such as a biomarker gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a biomarker gene under conditions such that hybridization and amplification of the biomarker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

In an alternative embodiment, mutations in a biomarker nucleic acid from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in biomarker nucleic acid can be identified by hybridizing a sample and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, biomarker genetic mutations can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene. Such biomarker genetic mutations can be identified in a variety of contexts, including, for example, germline and somatic mutations.

In yet another embodiment, any of a variety of sequencing reactions known in the art can be used to directly sequence a biomarker gene and detect mutations by comparing the sequence of the sample biomarker with the corresponding wild-type (control) sequence. Examples of sequencing reactions include those based on techniques developed by Maxam and Gilbert (1977) Proc. Natl. Acad. Sci. USA 74:560 or Sanger (1977) Proc. Natl. Acad Sci. USA 74:5463. It is also contemplated that any of a variety of automated sequencing procedures can be utilized when performing the diagnostic assays (Naeve (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a biomarker gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type biomarker sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in biomarker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a biomarker sequence, e.g., a wild-type biomarker treated with a DNA mismatch repair enzyme, and the cleavage products, if any, can be detected from electrophoresis protocols or the like (e.g., U.S. Pat. No. 5,459,039.)

In other embodiments, alterations in electrophoretic mobility can be used to identify mutations in biomarker genes. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat. Res. 285:125-144 and Hayashi (1992) Genet. Anal. Tech. Appl. 9:73-79). Single-stranded DNA fragments of sample and control biomarker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

e. Methods for Detection of Biomarker Epigenomic Alterations

The presence of hypermethylation or hypomethylation at genmic sites listed in Tables 1C and 1D can be detected and quantified by any of a number of means well-known to those of skill in the art. Such methods include, but are not limited to, differential enzymatic cleavage of DNA, digestion followed by PCR or sequencing, bisulfite conversion followed by methylation-specific PCR or sequencing, whole genome bisulfite sequencing (WGBS), PCR with high resolution melting, COLD-PCR for the detection of unmethylated islands, reduced representation bisulfite sequencing (RRBS), methyl-sensitive cut counting (MSCC), high performance liquid chromatography-ultraviolet (HPLC-UV), liquid chromatography coupled with tandem mass spectrometry (LC-MS/MS), ELISA, amplification fragment length polymorphism (AFLP), restriction fragment length polymorphism (RFLP), bead array (e.g., HumanMethylation450 BeadChip array), luminometric methylation assay (LUMA), LINE-1/pyrosequencing, and affinity capture of methylated DNA (Laird (2010) Nat. Rev. Genet. 11:191-203; Kurdyukov et al. (2016) Biology 5:3). Restriction enzyme based differential cleavage of methylated DNA may be locus-specific. Affinity-capture and bisulphite conversion followed by sequencing methods may be used for both gene specific or genome-wide analysis (Beck (2010) Nat. Biotech. 28:1026-1028). The commonly reported DNA affinity capture method may include methylated DNA immunoprecipitation (Me-DIP) that uses methyl DNA specific antibody, or methyl capture using methyl-CpG binding domain (MBD) proteins. In certain embodiment, whole genome bisulfite sequencing (WGBS) is used to detect the methylation level at genmic sites listed in Tables 1C and 1D.

### V. Anti-Cancer Therapies

Methods described herein can be used to assess whether a subject is afflicted with castration-resistant neuroendocrine prosate cancer (CRPC-NE) or at risk for developing CRPC-NE. In one embodiment, the subject is administered with an anti-cancer therapy (e.g., platinum-based chemotherapy) other than an AR-targeted therapy as a single agent if the subject is afflicted with CRPC-NE or at risk for developing CRPC-NE. In another embodiment, the subject is administered with an AR-targeted therapy if the subject is not afflicted with CRPC-NE or not at risk for developing CRPC-NE. The anti-cancer therapy is selected from the group consisting of an epgenitic modifier, targeted therapy, chemotherapy, radiation therapy, and/or hormonal therapy, optionally wherein the anti-cancer therapy comprises an AR-targeted therapy.

Combination therapies are also contemplated and can comprise, for example, one or more chemotherapeutic agents and radiation, one or more chemotherapeutic agents and immunotherapy, or one or more chemotherapeutic agents, radiation and chemotherapy, each combination of which can be with the AR-targeted therapy.

The term “targeted therapy” refers to administration of agents that selectively interact with a chosen biomolecule to thereby treat cancer. One example includes immunotherapies such as immune checkpoint inhibitors, which are well-known in the art. For example, anti-PD-1 pathway agents, such as therapeutic monoclonal blocking antibodies, which are well-known in the art and described above, can be used to target tumor microenvironments and cells expressing unwanted components of the PD-1 pathway, such as PD-1, PD-L1, and/or PD-L2.

For example, the term “PD-1 pathway” refers to the PD-1 receptor and its ligands, PD-L1 and PD-L2. “PD-1 pathway inhibitors” block or otherwise reduce the interaction between PD-1 and one or both of its ligands such that the immunoinhibitory signaling otherwise generated by the interaction is blocked or otherwise reduced. Anti-immune checkpoint inhibitors can be direct or indirect. Direct anti-immune checkpoint inhibitors block or otherwise reduce the interaction between an immune checkpoint and at least one of its ligands. For example, PD-1 inhibitors can block PD-1 binding with one or both of its ligands. Direct PD-1 combination inhibitors are well-known in the art, especially since the natural binding partners of PD-1 (e.g., PD-L1 and PD-L2), PD-L1 (e.g., PD-1 and B7-1), and PD-L2 (e.g., PD-1 and RGMb) are known.

For example, agents which directly block the interaction between PD-1 and PD-L1, PD-1 and PD-L2, PD-1 and both PD-L1 and PD-L2, such as a bispecific antibody, can prevent inhibitory signaling and upregulate an immune response (i.e., as a PD-1 pathway inhibitor). Alternatively, agents that indirectly block the interaction between PD-1 and one or both of its ligands can prevent inhibitory signaling and upregulate an immune response. For example, B7-1 or a soluble form thereof, by binding to a PD-L1 polypeptide indirectly reduces the effective concentration of PD-L1 polypeptide available to bind to PD-1. Exemplary agents include monospecific or bispecific blocking antibodies against PD-1, PD-L1, and/or PD-L2 that block the interaction between the receptor and ligand(s); a non-activating form of PD-1, PD-L1, and/or PD-L2 (e.g., a dominant negative or soluble polypeptide), small molecules or peptides that block the interaction between PD-1, PD-L1, and/or PD-L2; fusion proteins (e.g. the extracellular portion of PD-1, PD-L1, and/or PD-L2, fused to the Fc portion of an antibody or immunoglobulin) that bind to PD-1, PD-L1, and/or PD-L2 and inhibit the interaction between the receptor and ligand(s); a non-activating form of a natural PD-1, PD-L2, and/or PD-L2 ligand, and a soluble form of a natural PD-1, PD-L2, and/or PD-L2 ligand.

Indirect anti-immune checkpoint inhibitors block or otherwise reduce the immunoinhibitory signaling generated by the interaction between the immune checkpoint and at least one of its ligands. For example, an inhibitor can block the interaction between PD-1 and one or both of its ligands without necessarily directly blocking the interaction between PD-1 and one or both of its ligands. For example, indirect inhibitors include intrabodies that bind the intracellular portion of PD-1 and/or PD-L1 required to signal to block or otherwise reduce the immunoinhibitory signaling. Similarly, nucleic acids that reduce the expression of PD-1, PD-L1, and/or PD-L2 can indirectly inhibit the interaction between PD-1 and one or both of its ligands by removing the availability of components for interaction. Such nucleic acid molecules can block PD-L1, PD-L2, and/or PD-L2 transcription or translation.

Immunotherapies that are designed to elicit or amplify an immune response are referred to as “activation immunotherapies.” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In one embodiment, immunotherapy comprises adoptive cell-based immunotherapies. Well-known adoptive cell-based immunotherapeutic modalities, including, without limitation, Irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (MET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, patient-specific neoantigen vaccines, and the like.

In another embodiment, immunotherapy comprises non-cell-based immunotherapies. In one embodiment, compositions comprising antigens with or without vaccine-enhancing adjuvants are used. Such compositions exist in many well-known forms, such as peptide compositions, oncolytic viruses, recombinant antigen comprising fusion proteins, and the like. In still another embodiment, immunomodulatory interleukins, such as IL-2, IL-6, IL-7, IL-12, IL-17, IL-23, and the like, as well as modulators thereof (e.g., blocking antibodies or more potent or longer lasting forms) are used. In yet another embodiment, immunomodulatory cytokines, such as interferons, G-CSF, imiquimod, TNFalpha, and the like, as well as modulators thereof (e.g., blocking antibodies or more potent or longer lasting forms) are used. In another embodiment, immunomodulatory chemokines, such as CCL3, CCL26, and CXCL7, and the like, as well as modulators thereof (e.g., blocking antibodies or more potent or longer lasting forms) are used. In another embodiment, immunomodulatory molecules targeting immunosuppression, such as STAT3 signaling modulators, NFkappaB signaling modulators, and immune checkpoint modulators, are used. The terms “immune checkpoint” and “anti-immune checkpoint therapy” are described above.

Similarly, agents and therapies other than immunotherapy or in combination thereof can be used with in combination with biomarker inhibitor/immunotherapies to stimulate an immune response to thereby treat a condition that would benefit therefrom. For example, chemotherapy, radiation, epigenetic modifiers (e.g., histone deacetylase (HDAC) modifiers, methylation modifiers, phosphorylation modifiers, and the like), targeted therapy, and the like are well-known in the art.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, surgical intervention can occur to physically remove cancerous cells and/or tissues.

In still another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In yet another embodiment, hyperthermia, a procedure in which body tissue is exposed to high temperatures (up to 106° F.) is used. Heat may help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and can include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early non-small cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser—This type of laser can remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser—Light from this laser can penetrate deeper into tissue than light from the other types of lasers, and it can cause blood to clot quickly. It can be carried through optical fibers to less accessible parts of the body. This type of laser is sometimes used to treat throat cancers. Argon laser—This laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat seals blood vessels, there is less bleeding, swelling, or scarring. Laser surgery may be less complicated. For example, with fiber optics, laser light can be directed to parts of the body without making a large incision. More procedures may be done on an outpatient basis. Lasers can be used in two ways to treat cancer: by shrinking or destroying a tumor with heat, or by activating a chemical—known as a photosensitizing agent—that destroys cancer cells. In PDT, a photosensitizing agent is retained in cancer cells and can be stimulated by light to cause a reaction that kills cancer cells. CO₂ and Nd:YAG lasers are used to shrink or destroy tumors. They may be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems can produce a cutting area as small as 200 microns in diameter—less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

The duration and/or dose of treatment with therapies may vary according to the particular therapeutic agent or combination thereof. An appropriate treatment time for a particular cancer therapeutic agent will be appreciated by the skilled artisan. The present invention contemplates the continued assessment of optimal treatment schedules for each cancer therapeutic agent, where the phenotype of the cancer of the subject as determined by the methods of the present invention is a factor in determining optimal treatment doses and schedules.

Any means for the introduction of a polynucleotide into mammals, human or non-human, or cells thereof may be adapted to the practice of this invention for the delivery of the various constructs of the present invention into the intended recipient. In one embodiment of the present invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

The nucleic acids encoding a protein or nucleic acid of interest may be in a plasmid or viral vector, or other vector as is known in the art. Such vectors are well-known and any can be selected for a particular application. In one embodiment of the present invention, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter. A promoter may be constitutive or inducible.

In another embodiment, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al., J. Biol. Chem. 264:16985-16987, 1989), lipid-DNA combinations (Felgner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

### VI. Clinical Efficacy

Clinical efficacy can be measured by any method known in the art. For example, the response to a therapy, such as modulators of the genomic and/or epigenomic alterations of biomarkers described herein, relates to any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular anti-immune checkpoint therapeutic regimen is at least 25%, 30%, 35%, 40%, 45%, 50%, 55%, 60%, 65%, 70%, 75%, 80%, 85%, or more.

Additional criteria for evaluating the response to immunotherapies, such as anti-immune checkpoint therapies, are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

For example, in order to determine appropriate threshold values, a particular anti-cancer therapeutic regimen can be administered to a population of subjects and the outcome can be correlated to biomarker measurements that were determined prior to administration of any immunotherapy, such as anti-immune checkpoint therapy. The outcome measurement may be pathologic response to therapy given in the neoadjuvant setting. Alternatively, outcome measures, such as overall survival and disease-free survival can be monitored over a period of time for subjects following immunotherapies for whom biomarker measurement values are known. In certain embodiments, the same doses of immunotherapy agents, if any, are administered to each subject. In related embodiments, the doses administered are standard doses known in the art for those agents used in immunotherapies. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of an immunotherapy can be determined using methods such as those described in the Examples section.

### VII. Further Uses and Methods of the Present Invention

The compositions described herein can be used in a variety of diagnostic, prognostic, and therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

a. Screening Methods

One aspect of the present invention relates to screening assays, including non-cell based assays and xenograft animal model assays.

In one embodiment, the present invention relates to assays for screening test agents which modulate the genomic and/or epigenomic alteration(s) of, at least one biomarker described herein (e.g., in the tables, figures, examples, or otherwise in the specification). In one embodiment, a method for identifying such an agent entails determining the ability of the agent to modulate, the genomic and/or epigenomic alteration(s) of, at least one biomarker described herein.

In one embodiment, an assay is a cell-free or cell-based assay, comprising contacting at least one biomarker described herein, with a test agent, and determining the ability of the test agent to modulate the genomic and/or epigenomic alteration(s) of the biomarker, such as by measuring direct genomic and/or epigenomic alteration(s) of the biomarker or by measuring indirect parameters (e.g., by measuring mRNA expression, protein expression, and/or activity of the biomarker, and/or binding of the biomarker to its substrates).

The present invention further pertains to novel agents identified by the above-described screening assays. Accordingly, it is within the scope of this invention to further use an agent identified as described herein, such as in an appropriate animal model. For example, an agent identified as described herein can be used in an animal model to determine the efficacy, toxicity, or side effects of treatment with such an agent. Alternatively, an antibody identified as described herein can be used in an animal model to determine the mechanism of action of such an agent.

b. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining the genmic and/or epigenomic alterations of a biomarker described herein in the context of a biological sample (e.g., blood, serum, cells, or tissue) to thereby determine whether an individual afflicted with castration-resistant neuroendocrine prosate cancer (CRPC-NE) or at risk for developing CRPC-NE. Such assays can be used for prognostic or predictive purpose alone, or can be coupled with a therapeutic intervention to thereby prophylactically treat an individual prior to the onset or after recurrence of a disorder characterized by or associated with biomarker genmic and/or epigenomic alterations. The skilled artisan will appreciate that any method can use one or more (e.g., combinations) of biomarkers described herein, such as those in the tables, figures, examples, and otherwise described in the specification.

Another aspect of the present invention pertains to monitoring the influence of agents (e.g., drugs, compounds, and small nucleic acid-based molecules) on genmic and/or epigenomic alterations of a biomarker described herein. These and other agents are described in further detail in the following sections.

The skilled artisan will also appreciated that, in certain embodiments, the methods of the present invention implement a computer program and computer system. For example, a computer program can be used to perform the algorithms described herein. A computer system can also store and manipulate data generated by the methods of the present invention which comprises a plurality of biomarker signal changes/profiles which can be used by a computer system in implementing the methods of this invention. In certain embodiments, a computer system receives biomarker expression data; (ii) stores the data; and (iii) compares the data in any number of ways described herein (e.g., analysis relative to appropriate controls) to determine the state of informative biomarkers from cancerous or pre-cancerous tissue. In other embodiments, a computer system (i) compares the determined expression biomarker level to a threshold value; and (ii) outputs an indication of whether said biomarker level is significantly modulated (e.g., above or below) the threshold value, or a phenotype based on said indication.

In certain embodiments, such computer systems are also considered part of the present invention. Numerous types of computer systems can be used to implement the analytic methods of this invention according to knowledge possessed by a skilled artisan in the bioinformatics and/or computer arts. Several software components can be loaded into memory during operation of such a computer system. The software components can comprise both software components that are standard in the art and components that are special to the present invention (e.g., dCHIP software described in Lin et al. (2004) Bioinformatics 20, 1233-1240; radial basis machine learning algorithms (RBM) known in the art).

The methods of the present invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

In certain embodiments, the computer comprises a database for storage of biomarker data. Such stored profiles can be accessed and used to perform comparisons of interest at a later point in time. For example, biomarker expression profiles of a sample derived from the non-cancerous tissue of a subject and/or profiles generated from population-based distributions of informative loci of interest in relevant populations of the same species can be stored and later compared to that of a sample derived from the cancerous tissue of the subject or tissue suspected of being cancerous of the subject.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

c. Diagnostic Assays

The present invention provides, in part, methods, systems, and code for accurately classifying whether a biological sample (e.g., from a subject) is associated with castration-resistant neuroendocrine prosate cancer (CRPC-NE). In some embodiments, the present invention is useful for classifying a subject as associated with or at risk for developing castration-resistant neuroendocrine prosate cancer (CRPC-NE).

An exemplary method for detecting the genomic and/or epigenomic alterations of a biomarker described herein, and thus useful for classifying whether a subject is afflicted with castration-resistant neuroendocrine prosate cancer (CRPC-NE) or at risk for developing CRPC-NE. In certain instances, a neuroendocrine prostate cancer (NEPC) score is computed based on the presence or absence of one or more genomic or epigenomic alterations of the biomarkers described herein. In certain instances, the statistical algorithm is a single learning statistical classifier system. For example, a single learning statistical classifier system can be used to classify a subject as associated with or at risk for developing castration-resistant neuroendocrine prosate cancer (CRPC-NE) based upon a prediction or probability value and the presence or level of the genomic and/or epigenomic alterations of the biomarker. The use of a single learning statistical classifier system typically classifies the subject as, for example, associated with or at risk for developing castration-resistant neuroendocrine prosate cancer (CRPC-NE) with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the sample classification results to a clinician, e.g., an oncologist.

In another embodiment, the diagnosis of a subject is followed by administering to the individual a therapeutically effective amount of a defined treatment based upon the diagnosis. For example, in one embodiment, the subject is administered with an anti-cancer therapy (e.g., platinum-based chemotherapy) other than an AR-targeted therapy as a single agent if the subject is afflicted with CRPC-NE or at risk for developing CRPC-NE. In another embodiment, the subject is administered with an AR-targeted therapy if the subject is not afflicted with CRPC-NE or not at risk for developing CRPC-NE. The anti-cancer therapy is selected from the group consisting of an epgenitic modifier, targeted therapy, chemotherapy, radiation therapy, and/or hormonal therapy, optionally wherein the anti-cancer therapy comprises an AR-targeted therapy.

In one embodiment, the methods further involve obtaining a control biological sample (e.g., biological sample from a subject who does not have a prostate cancer or have castration resistant prostate adenocarcinoma (CRPC-Adeno)), a biological sample from the subject during remission, or a biological sample from the subject during treatment for developing a cancer progressing despite treatments with modulators of biomarker genomic and/or epigenomic alterations.

d. Prognostic Assays

The diagnostic methods described herein can furthermore be utilized to identify subjects having or at risk of developing CRPC-NE that is likely or unlikely to be responsive to modulators of biomarker genomic and/or epigenomic alterations. The assays described herein, such as the preceding diagnostic assays or the following assays, can be utilized to identify a subject having or at risk of developing a disorder associated with a misregulation of the genomic and/or epigenomic features of at least one biomarker described herein, such as in cancer. Alternatively, the prognostic assays can be utilized to identify a subject having or at risk for developing a disorder associated with a misregulation of the genomic and/or epigenomic features of at least one biomarker described herein, such as in cancer. Furthermore, the prognostic assays described herein can be used to determine whether a subject can be administered an agent (e.g., an agonist, antagonist, peptidomimetic, polypeptide, peptide, nucleic acid, small molecule, an epigenetic modifier, or other drug candidate) to treat a disease or disorder associated with the aberrant biomarker genomic and/or epigenomic features.

e. Treatment Methods

The therapeutic compositions described herein, such as agents modulating genomic and/or epigenomic alterations of biomarkers described herein, can be used in a variety of in vitro and in vivo therapeutic applications using the formulations and/or combinations described herein. In one embodiment, the therapeutic agents can be used to treat CRPC-NE cancer. For example, single or multiple agents that modulate genomic and/or epigenomic alterations of a biomarker alone or in combination with an additional anti-cancer therapy (e.g., chemotherapy, immunotherapy, or AR-targeted therapy) can be used to treat cancers in subjects identified as having or at the risk of developing CRPC-NE.

### VIII. Administration of Agents

In another aspect, the present invention provides pharmaceutically acceptable compositions which comprise a therapeutically-effective amount of an agent that modulates biomarker genomic and/or epigenmic alterations described herein, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “therapeutically-effective amount” as used herein means that amount of an agent that modulates (e.g., inhibits) biomarker expression and/or activity, or expression and/or activity of the complex, or composition comprising an agent that modulates (e.g., inhibits) biomarker expression and/or activity, or expression and/or activity of the complex, which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) biomarker expression and/or activity, or expression and/or activity of the complex encompassed by the present invention. These salts can be prepared in situ during the final isolation and purification of the agents, or by separately reacting a purified agent in its free base form with a suitable organic or inorganic acid, and isolating the salt thus formed. ReRepresentative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) biomarker expression and/or activity, or expression and/or activity of the complex. These salts can likewise be prepared in situ during the final isolation and purification of the agents, or by separately reacting the purified agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. ReRepresentative alkali or alkaline earth salts include the lithium, sodium, potassium, calcium, magnesium, and aluminum salts and the like. ReRepresentative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates (e.g., inhibits) biomarker expression and/or activity, with the carrier and, optionally, one or more accessory ingredients. In general, the formulations are prepared by uniformly and intimately bringing into association a agent with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a agent as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of an agent that modulates (e.g., inhibits) biomarker expression and/or activity include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) biomarker expression and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates (e.g., inhibits) biomarker expression and/or activity, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., inhibits) biomarker expression and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the agents of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

The nucleic acid molecules of the present invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054 3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

### IX. Kits

The present invention also encompasses kits for detecting and/or modulating biomarkers described herein. A kit of the present invention may also include instructional materials disclosing or describing the use of the kit or an antibody of the disclosed invention in a method of the disclosed invention as provided herein. A kit may also include additional components to facilitate the particular application for which the kit is designed. For example, a kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents recognized for use in a method of the disclosed invention. Non-limiting examples include agents to reduce non-specific binding, such as a carrier protein or a detergent.

### EXAMPLES

### Example 1: Materials and Methods for Example 2

a. Clinical Cohort

Patients were prospectively enrolled on an IRB approved protocol (#1305013903) with written informed consent. Patients were eligible for this study if they had metastatic prostate cancer with at least one metastatic tumor biopsy and matched plasma sample (EDTA or Streck Cell-Free DNA BCT®) and at least 2-50 ng of total cell free DNA (cfDNA) present in 1 mL of plasma. Whole exome sequencing (WES) of metastatic biopsy was previously reported for 53/63 patients (Beltran et al. (2016) Nat Med 22:298-305; Beltran et al. (2018) Clin Cancer Res 25:43-51). Peripheral blood samples were approximately collected within 30 days of treatment initiation (in ˜85% of cases). The choice of systemic therapies before plasma sample collection was at the discretion of the treating physician. Treatments were administered continuously until evidence of progression disease, unacceptable toxicity, or for a planned number of cycles in the case of chemotherapy according to regime-specific treatment protocols. Patients treated with at least one month duration on treatment were included, defined as “the duration of time from initiation to discontinuation of therapy”. Clinical data was obtained from the electronic medical record. Pathology review was performed and reported as adenocarcinoma or CRPC-NE using published morphologic criteria (Epstein et al. (2014) Am J Surg Pathol 38:756-767).

b. Plasma Processing and DNA Extraction and Quantification

Whole blood was centrifuged at 1,600 g×10 minutes at 4° C. within 3 hours after blood collection. The plasma layer was transferred to 2 ml microcentrifuge tubes and centrifuged at 16,000 g×10 minutes at 4° C. to ensure removal of any cellular debris. The plasma was then collected and stored at −80° C. cfDNA was extracted from plasma using the NeoGeneStar Cell-Free DNA Purification kit per manufacturer's instructions (Somerset, N.J.). Briefly, for 2 ml plasma samples, cfDNA was isolated via proteolytic digestion with 1 μg of RNA carrier at 55° C. for 30 minutes. cfDNA capture on the superparamagnetic particles was accomplished via addition of 3 volumes (6 mL) of LYS buffer, 0.8 mL isopropanol and 30 μL NGS™ Beads, capture of cfDNA via 30 minute room temperature incubation, 2× wash and 2×80% EtOH, air dry, and elution in 30 μL 10 mM Tris, 0.1 mM EDTA (pH 8.5). Germline DNA was extracted from extracellular blood components (PBMC) using the Promega Maxwell 16 MDx kit per manufacturer's instructions. Blood aliquots are vortexed briefly and mixed with LYS buffer and proteinase K solution, vortexed briefly again, and incubated at 56° C. for 20 minutes. Lysates are then transferred to the first well of a multiwall cartridge prefilled with reagents, 65 μl of elution buffer is loaded, and the automated system successfully isolates high concentration genomic DNA. cfDNA was quantified with Qubit and quality also assessed by using Agilent's High Sensitivity DNA kit.

c. Whole exome sequencing of ctDNA and matched PBMC DNA

Based on the expected circulating DNA fragment size distribution and the range of input DNA identified from each plasma sample, the Roche SeqCap EZ Library SR (Pleasanton, Calif.) was opted for library preparation. Germline genomic DNA was sheared using the Covaris E220 Evolution instrument (Covaris, Woburn, Mass.). For cfDNA, input for library prep ranged from 2.8-50 ng and for germline DNA input ranged from 50-100 ng. The libraries of both germline DNA and cfDNA were prepared using the KAPA HTP Library Preparation Protocol (Kapa Biosystems, Wilmington, Mass.) containing end repair, A-base addition, ligation of sequence adaptors. The sample libraries were normalized and pooled following PCR amplification. Then the pooled libraries were hybridized with whole exome SeqCap EZ probe pool. Finally, the pooled, indexed and amplified capture sample libraries were sequenced using the Illumina HiSeq4000 sequencer at 100 cycles (San Diego, Calif.). All plasma and matched normal samples were run with single end protocol unless differently specified. Illumina bcl2fastq2 Conversion Software was used to demultiplex samples into individual sample and converted per-cycle BCL base call files into FASTQ files for downstream data analysis. WES sample processing and data generation procedure of tissue biopsies sequenced in this study follows the same protocol as in Beltran et al. (2016) Nat Med 22:298-305; all tissues were run with paired end protocol.

d. Titration Experiment to Determine Optimal ctDNA Input for WES

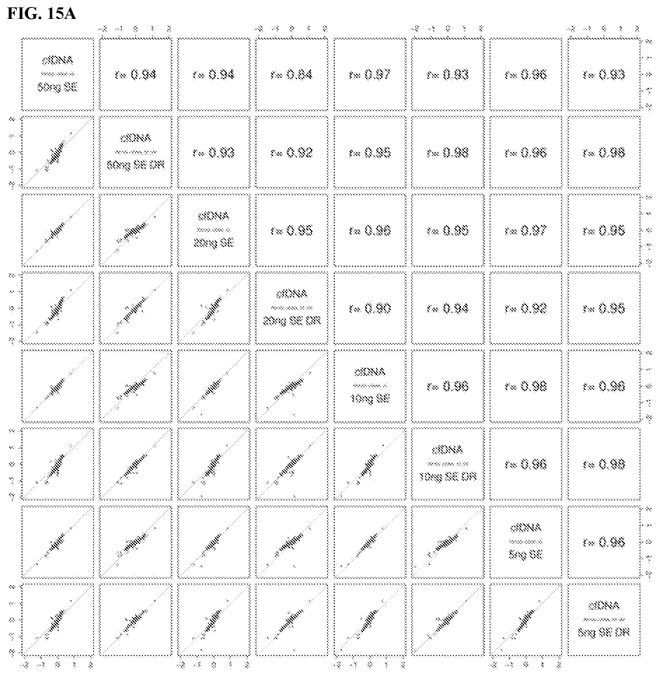

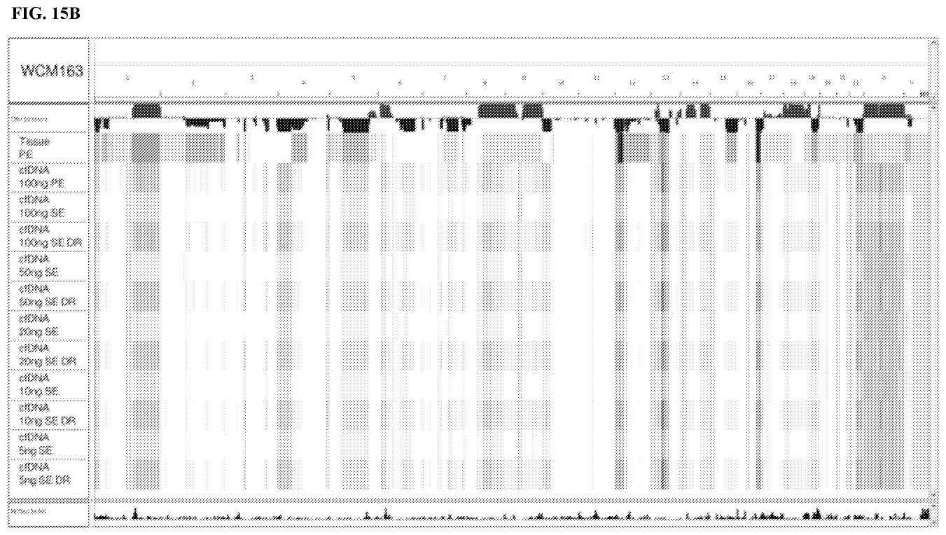

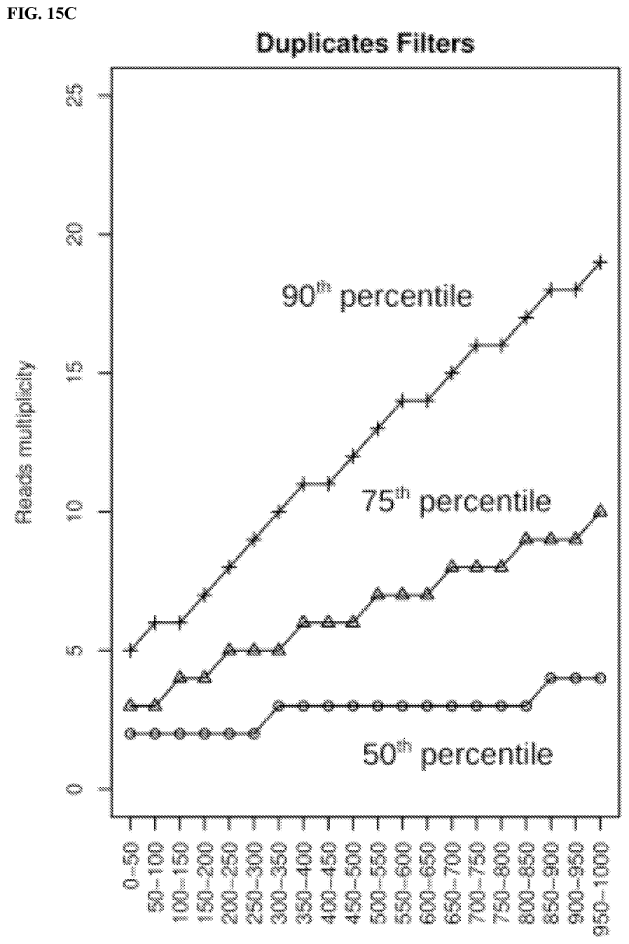

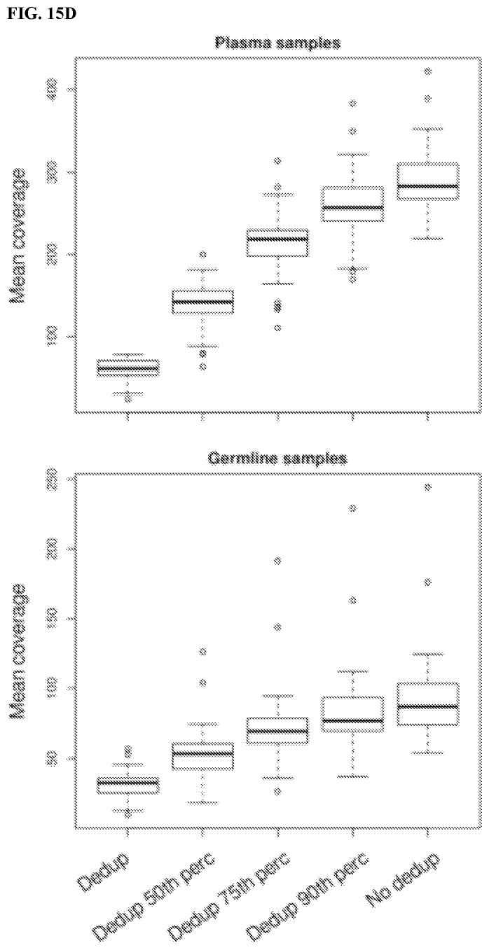

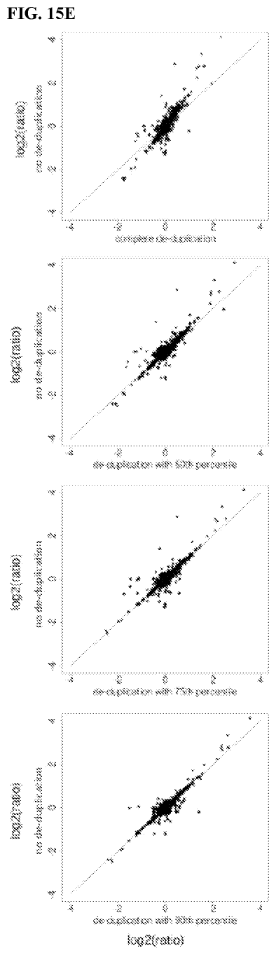

Prior to profiling the whole cfDNA study cohort, the effect of input amount for cfDNA on the sequencing based genomic profiles was studied. One patient with known genotype features (WCM163) was selected for this experiment. Specifically, the cfDNA was diluted into five concentrations using 5 ng, 10 ng, 20 ng, 50 ng, and 100 ng. For matched germline DNA, 100 ng was used for all comparisons. All downstream library preparation and sequencing were conducted as stated above. Somatic copy number calls of 1000 cancer associated genes were compared (FIGS. 15A-15B).

e. WES Processing Pipeline

(i) Data Pre-Processing

Tumor tissues BAM files were generated through the WCM Englander Institute of Precision Medicine pipeline (Beltran et al. (2015) JAMA Oncol 1:466-474) and pre-processed at the University of Trento. As per the cfDNA samples and matched germline DNA samples, the FastQC tool (available on the World Wide Web at bioinformatics.babraham.ac.uk/projects/fastqc) was run on the raw reads to assess their quality; quality metrics include average base quality, sequence duplication rate, and the k-mer enrichment along the length of the reads. These measures were utilized to assess whether the sequencing and the de-multiplexing of the samples was performed correctly. After initial quality control, adapter sequences were trimmed using Trimmomatic (Bolger, Lohse, and Usadel (2014) Bioinformatics 30:2114-2120). Short reads were then aligned to GRC37/hg19 reference using BWA (Li and Durbin (2009) Bioinformatics 25:1754-1760). Picard (available on the World Wide Web at broadinstitute.github.io/picard/) and SAMtools (Li et al. (2009) Bioinformatics 25:2078-2079) were used to generate single sorted BAM files for each patient's sample. BAM files were then realigned (to correct for possible misalignments due to indels) and recalibrated (to adjust for over- and under-estimated base quality scores in the data) using GATK standard pipelines (McKenna et al. (2010) Genome Res 20:1297-1303). Finally, SAMtools were used to adjust BAM MD tags (strings for mismatch positions). The alignment quality of the BAM files was obtained by several metrics related to the average coverage and capture rate to calculate how many aligned reads fall within a capture region of the Nimblegen SeqCap EZ Exome V3 kit. For any given sample, the capture rate was given by the percent of mapped reads that overlap any capture region in the kit and the total number of mapped reads. Average coverage was computed from the captured regions of the Nimblegen kit. Metrics from 62 processed patients' data showed that average coverage for plasma DNA and germline DNA was 361× and 109×, respectively; average capture rates were 81% for plasma DNA and 77% for germline DNA (Table 2). All subsequent steps were applied to all samples, tissues and cfDNA, unless differently specified.

TABLE 2
Sequencing statistics of plasma genomics

| | | | | | | | | | | Mean |
|---|---|---|---|---|---|---|---|---|---|---|
| | | | | | | | | | | coverage |
| | | | | | Mean | | | | | in |
| | | | | | coverage | | | Reads | Fraction | captured |
| | | | | | in | | Tot reads | captured | captured | regions |
| | | | Reads | Fraction | captured | Duplication | after | after | after | after |
| Sample | Type | Tot reads | captured | captured | regions | fraction | dedup | dedup | dedup | dedup |
| WCM0 | Plasma | 311122716 | 249173112 | 0.806 | 309 | 0.702 | 92846793 | 66369357 | 0.72 | 81 |
| WCM0 | Germline | 132057629 | 108314704 | 0.825 | 136 | 0.745 | 33704485 | 26242119 | 0.784 | 33 |
| WCM1 | Plasma | 364378360 | 305606474 | 0.844 | 383 | 0.784 | 78807505 | 60032334 | 0.767 | 74 |
| WCM1 | Germline | 134725406 | 97302274 | 0.727 | 118 | 0.527 | 63666839 | 43211401 | 0.684 | 52 |
| WCM10 | Plasma | 325258174 | 268987483 | 0.832 | 336 | 0.812 | 61283551 | 46755700 | 0.768 | 57 |
| WCM10 | Germline | 157894805 | 123979502 | 0.79 | 153 | 0.706 | 46487109 | 35302916 | 0.764 | 43 |
| WCM119 | Plasma | 339732023 | 281206149 | 0.833 | 352 | 0.778 | 75507734 | 56785036 | 0.757 | 70 |
| WCM119 | Germline | 102408298 | 79851989 | 0.785 | 99 | 0.563 | 44754960 | 32843646 | 0.739 | 41 |
| WCM12 | Plasma | 146838947 | 114718671 | 0.786 | 145 | 0.52 | 70486563 | 49513622 | 0.708 | 62 |
| WCM12 | Germline | 113199598 | 84472341 | 0.751 | 105 | 0.382 | 69924342 | 49088484 | 0.707 | 61 |
| WCM121 | Plasma | 377493251 | 307550034 | 0.82 | 382 | 0.818 | 68598741 | 50869345 | 0.746 | 62 |
| WCM121 | Germline | 91940399 | 70836961 | 0.776 | 87 | 0.54 | 42289477 | 31556839 | 0.751 | 39 |
| WCM124 | Plasma | 329458636 | 264249102 | 0.807 | 330 | 0.71 | 95647244 | 67145200 | 0.707 | 82 |
| WCM124 | Germline | 114529272 | 89451053 | 0.786 | 112 | 0.597 | 46159057 | 33554082 | 0.732 | 42 |
| WCM131 | Plasma | 332482667 | 272390697 | 0.824 | 339 | 0.813 | 61995026 | 46344122 | 0.752 | 57 |
| WCM131 | Germline | 92823348 | 71135220 | 0.771 | 88 | 0.551 | 41644766 | 30802749 | 0.745 | 38 |
| WCM1388 | Plasma | 257042780 | 183395420 | 0.718 | 233 | 0.595 | 104156766 | 61612998 | 0.596 | 77 |
| WCM139 | Plasma | 401126385 | 329103870 | 0.825 | 409 | 0.829 | 68441314 | 51128859 | 0.752 | 62 |
| WCM139 | Germline | 102066172 | 76920197 | 0.759 | 95 | 0.535 | 47451349 | 34310233 | 0.728 | 42 |
| WCM14 | Germline | 88651447 | 71739448 | 0.814 | 114 | 0.43 | 50539645 | 39530404 | 0.787 | 64 |
| WCM14- | Plasma | 267237615 | 205486181 | 0.774 | 256 | 0.807 | 51714781 | 36273402 | 0.706 | 45 |
| TP1 | | | | | | | | | | |
| WCM14- | Plasma | 431618248 | 317265361 | 0.74 | 394 | 0.834 | 71472297 | 47583092 | 0.671 | 59 |
| TP2 | | | | | | | | | | |
| WCM14- | Plasma | 444003297 | 340281994 | 0.771 | 434 | 0.831 | 75162329 | 50598425 | 0.678 | 63 |
| TP3 | | | | | | | | | | |
| WCM14- | Plasma | 452414260 | 342456465 | 0.762 | 432 | 0.828 | 77886332 | 52199758 | 0.675 | 65 |
| TP4 | | | | | | | | | | |
| WCM142 | Plasma | 368352710 | 309316542 | 0.845 | 387 | 0.804 | 72190734 | 55448922 | 0.773 | 68 |
| WCM142 | Germline | 116039055 | 92768415 | 0.804 | 117 | 0.647 | 40928654 | 29763588 | 0.732 | 37 |
| WCM15 | Plasma | 364394194 | 302697312 | 0.836 | 379 | 0.812 | 68651241 | 52682021 | 0.772 | 65 |
| WCM15 | Germline | 94349727 | 71561324 | 0.764 | 89 | 0.576 | 39959042 | 29145825 | 0.734 | 36 |
| WCM154 | Plasma | 413870150 | 339821702 | 0.826 | 426 | 0.787 | 88038344 | 64765039 | 0.741 | 79 |
| WCM154 | Germline | 101422976 | 77663634 | 0.771 | 96 | 0.538 | 46896021 | 34449792 | 0.74 | 43 |
| WCM155 | Plasma | 350918758 | 287405648 | 0.824 | 361 | 0.724 | 96788634 | 70503776 | 0.733 | 87 |
| WCM155 | Germline | 156304396 | 129585473 | 0.834 | 163 | 0.738 | 40876059 | 32358950 | 0.797 | 40 |
| WCM156 | Plasma | 333582149 | 266044119 | 0.802 | 330 | 0.837 | 54380948 | 39813523 | 0.737 | 49 |
| WCM156 | Germline | 116725152 | 94825184 | 0.817 | 119 | 0.758 | 28202556 | 21958892 | 0.784 | 27 |
| WCM158 | Plasma | 380118488 | 306725227 | 0.812 | 382 | 0.801 | 75743656 | 54083211 | 0.719 | 66 |
| WCM158 | Germline | 98417749 | 75346177 | 0.771 | 93 | 0.522 | 47078980 | 34654706 | 0.741 | 43 |
| WCM160 | Plasma | 361020626 | 302887592 | 0.844 | 380 | 0.799 | 72555902 | 55613548 | 0.772 | 68 |
| WCM160 | Germline | 115411842 | 86946093 | 0.758 | 107 | 0.557 | 51123404 | 36138223 | 0.712 | 44 |
| WCM161 | Plasma | 320462223 | 256166282 | 0.804 | 318 | 0.709 | 93225952 | 65727627 | 0.71 | 80 |
| WCM161 | Germline | 136355589 | 103139550 | 0.761 | 127 | 0.563 | 59644595 | 42402294 | 0.716 | 52 |
| WCM162 | Plasma | 317326927 | 260507673 | 0.826 | 328 | 0.707 | 93038914 | 68037407 | 0.736 | 84 |
| WCM162 | Germline | 129958304 | 101506344 | 0.786 | 125 | 0.73 | 35055959 | 26512045 | 0.761 | 33 |
| WCM163 | Plasma | 388446272 | 335685040 | 0.869 | 427 | 0.8 | 77693118 | 62747957 | 0.813 | 78 |
| WCM163 | Germline | 282466085 | 241607179 | 0.86 | 305 | 0.787 | 60099767 | 48163405 | 0.806 | 60 |
| WCM164 | Plasma | 346434795 | 276372383 | 0.803 | 341 | 0.822 | 61770539 | 45482792 | 0.741 | 55 |
| WCM164 | Germline | 139483439 | 107088863 | 0.773 | 132 | 0.787 | 29726880 | 22057320 | 0.747 | 27 |
| WCM169 | Plasma | 341180505 | 271379544 | 0.8 | 336 | 0.77 | 78644539 | 56166872 | 0.719 | 68 |
| WCM169 | Germline | 152231431 | 122050275 | 0.807 | 152 | 0.768 | 35277083 | 26946654 | 0.769 | 33 |
| WCM170 | Plasma | 437197105 | 350617744 | 0.807 | 436 | 0.843 | 68819728 | 50567796 | 0.74 | 62 |
| WCM170 | Germline | 127504894 | 97860652 | 0.772 | 120 | 0.814 | 23728212 | 17464856 | 0.741 | 22 |
| WCM172 | Plasma | 359396289 | 288491831 | 0.808 | 357 | 0.767 | 83771387 | 60094618 | 0.722 | 73 |
| WCM172 | Germline | 73367823 | 54074003 | 0.742 | 67 | 0.514 | 35669665 | 25363022 | 0.716 | 31 |
| WCM176 | Plasma | 352608174 | 284271899 | 0.811 | 354 | 0.874 | 44272363 | 28694330 | 0.653 | 34 |
| WCM176 | Germline | 111450347 | 90401974 | 0.816 | 112 | 0.67 | 36805491 | 28884062 | 0.79 | 36 |
| WCM183 | Plasma | 379752487 | 308316924 | 0.817 | 384 | 0.741 | 98393037 | 70348682 | 0.72 | 86 |
| WCM183 | Germline | 128306159 | 101267651 | 0.794 | 127 | 0.61 | 50020410 | 36823192 | 0.741 | 46 |
| WCM185 | Plasma | 525551626 | 423112703 | 0.81 | 527 | 0.817 | 96033494 | 69124082 | 0.725 | 84 |
| WCM185 | Germline | 99013894 | 77896867 | 0.792 | 96 | 0.77 | 22785954 | 16993419 | 0.751 | 21 |
| WCM185- | Plasma | 336871889 | 261840260 | 0.782 | 328 | 0.84 | 53770438 | 37818640 | 0.708 | 47 |
| TP1 | | | | | | | | | | |
| WCM185- | Plasma | 363124324 | 277126434 | 0.768 | 347 | 0.838 | 58886645 | 40516476 | 0.693 | 50 |
| TP2 | | | | | | | | | | |
| WCM185- | Plasma | 378894036 | 279387694 | 0.742 | 348 | 0.844 | 59028730 | 38738043 | 0.661 | 48 |
| TP3 | | | | | | | | | | |
| WCM185- | Plasma | 407324089 | 313511254 | 0.775 | 394 | 0.84 | 65168852 | 44818774 | 0.693 | 56 |
| TP4 | | | | | | | | | | |
| WCM192 | Plasma | 384414155 | 308313856 | 0.807 | 385 | 0.812 | 72193475 | 53011960 | 0.739 | 65 |
| WCM192 | Germline | 136760982 | 99438480 | 0.732 | 120 | 0.76 | 32879233 | 23138910 | 0.709 | 28 |
| WCM195 | Plasma | 357646622 | 297394829 | 0.836 | 375 | 0.777 | 79619541 | 60251400 | 0.762 | 75 |
| WCM195 | Germline | 82551231 | 59966483 | 0.731 | 74 | 0.445 | 45830606 | 31567261 | 0.694 | 39 |
| WCM198 | Plasma | 330615193 | 275542221 | 0.838 | 350 | 0.783 | 71758779 | 54351419 | 0.762 | 68 |
| WCM198 | Germline | 145254551 | 102693130 | 0.712 | 125 | 0.457 | 78928376 | 52206844 | 0.666 | 63 |
| WCM199 | Plasma | 349870338 | 279756758 | 0.805 | 348 | 0.787 | 74377712 | 53383885 | 0.723 | 65 |
| WCM199 | Germline | 73165515 | 53604497 | 0.738 | 67 | 0.393 | 44409836 | 31166112 | 0.707 | 38 |
| WCM2 | Plasma | 382980081 | 315077922 | 0.828 | 393 | 0.806 | 74148650 | 56102263 | 0.762 | 69 |
| WCM2 | Germline | 91146404 | 65561628 | 0.724 | 80 | 0.598 | 36626465 | 25446296 | 0.7 | 31 |
| WCM201 | Plasma | 402714823 | 307603800 | 0.769 | 379 | 0.827 | 69714756 | 46623062 | 0.674 | 57 |
| WCM201 | Germline | 78864621 | 61573953 | 0.786 | 77 | 0.375 | 49283693 | 37016542 | 0.756 | 46 |
| WCM207 | Plasma | 325803297 | 276547327 | 0.854 | 350 | 0.781 | 71367562 | 55651835 | 0.785 | 69 |
| WCM207 | Germline | 119213126 | 90358149 | 0.763 | 112 | 0.59 | 48938220 | 34467698 | 0.709 | 42 |
| WCM212 | Plasma | 391379376 | 319619872 | 0.822 | 404 | 0.793 | 80878112 | 59630513 | 0.742 | 74 |
| WCM212 | Germline | 69578577 | 51716474 | 0.748 | 65 | 0.391 | 42365654 | 30168754 | 0.717 | 38 |
| WCM213 | Plasma | 341951707 | 286459655 | 0.843 | 358 | 0.792 | 71297743 | 54244071 | 0.766 | 66 |
| WCM213 | Germline | 85531748 | 63262393 | 0.745 | 78 | 0.521 | 40953823 | 28185040 | 0.693 | 35 |
| WCM216 | Plasma | 399678023 | 320815070 | 0.808 | 398 | 0.788 | 84739921 | 58463964 | 0.695 | 72 |
| WCM216 | Germline | 134810654 | 87561520 | 0.655 | 107 | 0.352 | 87354881 | 51952767 | 0.6 | 63 |
| WCM218 | Plasma | 270804563 | 226725381 | 0.842 | 284 | 0.768 | 62896545 | 48106742 | 0.77 | 59 |
| WCM218 | Germline | 95995138 | 71825506 | 0.753 | 89 | 0.528 | 45362309 | 31755524 | 0.705 | 39 |
| WCM219 | Plasma | 295904118 | 232966360 | 0.792 | 288 | 0.814 | 55004409 | 38729540 | 0.709 | 48 |
| WCM219 | Germline | 98077887 | 76062103 | 0.78 | 95 | 0.427 | 56187677 | 41708133 | 0.747 | 52 |
| WCM223 | Plasma | 401926996 | 313797098 | 0.786 | 389 | 0.802 | 79608094 | 54703327 | 0.692 | 67 |
| WCM223 | Germline | 89279610 | 68961199 | 0.777 | 86 | 0.413 | 52433347 | 38706969 | 0.743 | 48 |
| WCM243 | Plasma | 332907337 | 262511887 | 0.794 | 323 | 0.737 | 87453344 | 60845148 | 0.701 | 74 |
| WCM243 | Germline | 109125850 | 82253426 | 0.759 | 101 | 0.841 | 17326455 | 12101985 | 0.703 | 15 |
| WCM247 | Plasma | 369235160 | 294445463 | 0.802 | 368 | 0.76 | 88618869 | 61551654 | 0.7 | 75 |
| WCM247 | Germline | 175875262 | 138562855 | 0.793 | 173 | 0.814 | 32753419 | 22482644 | 0.691 | 27 |
| WCM252 | Plasma | 397034260 | 336504861 | 0.852 | 426 | 0.781 | 86951082 | 67064520 | 0.776 | 83 |
| WCM252 | Germline | 111621700 | 85526634 | 0.771 | 107 | 0.576 | 47379534 | 33894485 | 0.72 | 42 |
| WCM253 | Plasma | 349335311 | 276009548 | 0.795 | 342 | 0.787 | 74422557 | 51128968 | 0.692 | 62 |
| WCM253 | Germline | 172203151 | 135343920 | 0.791 | 169 | 0.788 | 36498615 | 25505416 | 0.704 | 31 |
| WCM261 | Plasma | 456866372 | 367388703 | 0.809 | 455 | 0.798 | 92430033 | 63948233 | 0.697 | 78 |
| WCM261 | Germline | 68958271 | 48396275 | 0.707 | 61 | 0.323 | 46667403 | 31010467 | 0.67 | 39 |
| WCM341 | Plasma | 412880192 | 329820557 | 0.804 | 409 | 0.767 | 96245678 | 67457090 | 0.706 | 83 |
| WCM341 | Germline | 87596583 | 59815812 | 0.688 | 74 | 0.337 | 58062736 | 37359850 | 0.648 | 46 |
| WCM365 | Plasma | 431976173 | 340308459 | 0.793 | 426 | 0.794 | 89131325 | 61519089 | 0.695 | 76 |
| WCM365 | Germline | 95101752 | 72668341 | 0.769 | 91 | 0.401 | 56994975 | 41359034 | 0.731 | 51 |
| WCM366 | Plasma | 305675512 | 235722635 | 0.776 | 293 | 0.798 | 61642577 | 42605631 | 0.696 | 52 |
| WCM366 | Germline | 94904757 | 71028303 | 0.753 | 88 | 0.391 | 57802427 | 41093684 | 0.716 | 51 |
| WCM369 | Plasma | 392206305 | 307159082 | 0.788 | 384 | 0.798 | 79419300 | 54862421 | 0.696 | 67 |
| WCM369 | Germline | 81574616 | 61602446 | 0.76 | 77 | 0.367 | 51655125 | 37287336 | 0.727 | 46 |
| WCM4 | Plasma | 349741918 | 287494050 | 0.827 | 358 | 0.812 | 65574106 | 49221059 | 0.756 | 60 |
| WCM4 | Germline | 136167012 | 105295507 | 0.778 | 129 | 0.715 | 38859819 | 29132187 | 0.755 | 36 |
| WCM6 | Plasma | 317200728 | 262681116 | 0.833 | 328 | 0.789 | 66791492 | 51019927 | 0.769 | 63 |
| WCM6 | Germline | 115587780 | 95627214 | 0.832 | 120 | 0.823 | 20402683 | 15258372 | 0.753 | 19 |
| WCM63 | Plasma | 264592541 | 218381370 | 0.83 | 271 | 0.832 | 44383911 | 33611154 | 0.762 | 41 |
| WCM63 | Germline | 87498006 | 61719919 | 0.71 | 76 | 0.585 | 36346776 | 24827171 | 0.688 | 30 |
| WCM677 | Plasma | 335742996 | 270110809 | 0.81 | 339 | 0.82 | 60590733 | 43838656 | 0.728 | 54 |
| WCM677 | Germline | 93862570 | 70646764 | 0.758 | 88 | 0.393 | 56965506 | 40769042 | 0.721 | 50 |
| WCM7 | Plasma | 316062922 | 257857266 | 0.821 | 324 | 0.705 | 93212502 | 67504147 | 0.729 | 83 |
| WCM7 | Germline | 97224429 | 74569077 | 0.772 | 93 | 0.598 | 39095858 | 27751560 | 0.715 | 34 |
| WCM73 | Plasma | 294754578 | 227594216 | 0.777 | 280 | 0.842 | 46554227 | 32695790 | 0.707 | 40 |
| WCM73 | Germline | 119236025 | 89389895 | 0.755 | 109 | 0.735 | 31581338 | 22906218 | 0.73 | 28 |
| WCM78 | Plasma | 460196208 | 383499927 | 0.838 | 480 | 0.829 | 78503572 | 59915855 | 0.768 | 74 |
| WCM78 | Germline | 100073107 | 78633474 | 0.791 | 98 | 0.578 | 42281085 | 31987986 | 0.762 | 40 |
| WCM80 | Plasma | 352697630 | 272591824 | 0.778 | 335 | 0.845 | 54657927 | 38416578 | 0.708 | 47 |
| WCM80 | Germline | 134656931 | 104496575 | 0.781 | 129 | 0.698 | 40596881 | 30524576 | 0.757 | 38 |
| WCM89 | Plasma | 391524851 | 321014747 | 0.825 | 400 | 0.827 | 67632934 | 50118395 | 0.746 | 61 |
| WCM89 | Germline | 87618661 | 70667991 | 0.812 | 88 | 0.528 | 41356826 | 32360127 | 0.788 | 40 |
| WCM9 | Plasma | 300828453 | 249127616 | 0.833 | 310 | 0.789 | 63403107 | 48376079 | 0.768 | 59 |
| WCM9 | Germline | 153725403 | 122679104 | 0.803 | 151 | 0.694 | 46989357 | 36197890 | 0.775 | 44 |
| WCM90 | Plasma | 288566429 | 236690670 | 0.825 | 297 | 0.69 | 89560896 | 65987775 | 0.742 | 81 |
| WCM90 | Germline | 99948298 | 76273435 | 0.768 | 95 | 0.539 | 46115597 | 33829674 | 0.739 | 42 |
| WCM91 | Plasma | 353999832 | 272524812 | 0.775 | 334 | 0.833 | 59005760 | 41727140 | 0.712 | 50 |
| WCM91 | Germline | 129574648 | 97500264 | 0.758 | 119 | 0.847 | 19797087 | 13992622 | 0.712 | 17 |

(ii) Sample's Identity Check

In order to verify the correct match of all individual's samples, a solid genotype distance based test was applied, SPIA (Demichelis et al. (2008) Nucleic Acids Res 36:2446-2456) exploiting the related R package SPIAssay. Genotypes of 334 selected SNPs were computed using ASEQ (Romanel et al. (2015) BMC Med Genomics 8:9). The 334 SNPs were previously selected based on the minor allele frequency (MAF) and uniform distribution across the genome.

(iii) Optimized De-Duplication Procedure for WES Plasma Data from Single End

Protocol and with Deep Coverage WES data generated from both germline and cfDNA study samples showed high fractions of read duplicates in the sample cohort (mean 59% and 79%, respectively). Anti-correlation between duplication percentage and input DNA was observed (−0.83 Pearson coefficient, P=9e−04). Duplication levels in off-target regions were comparable to on-target region. Provided that without using specific technologies (e.g. molecular barcoding) it is not possible to measure and quantify the exact proportions of PCR and natural duplication (due to high-coverage), an ad-hoc computational solution that limits the impact of non-natural reads duplicates in down-stream copy number analyses was opted for. First, the empirical distribution of reads duplicates at different coverage intervals from all samples of the cohort was measured. Then each coverage interval the 50th, 75th and 90th percentiles were collected and these percentiles were used across all coverage intervals to create three different duplication thresholds (FIG. 15C). Finally, these thresholds were applied when the coverage statistics at single base resolution is computed for down-stream analysis. Specifically, when coverage statistics are computed for a position x in the genome, the raw coverage C is first computed considering all reads and is then normalized considering the duplication threshold T specific for C. Coverage statistics were computed for all three 50th, 75th and 90th percentiles thresholds. Coverage distributions obtained applying the different duplication thresholds are shown in (FIG. 15D).

(iv) WES Segmentation

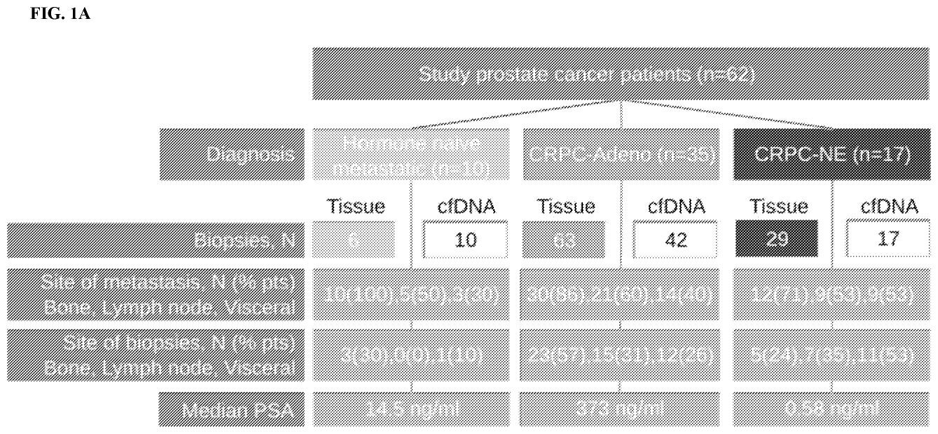

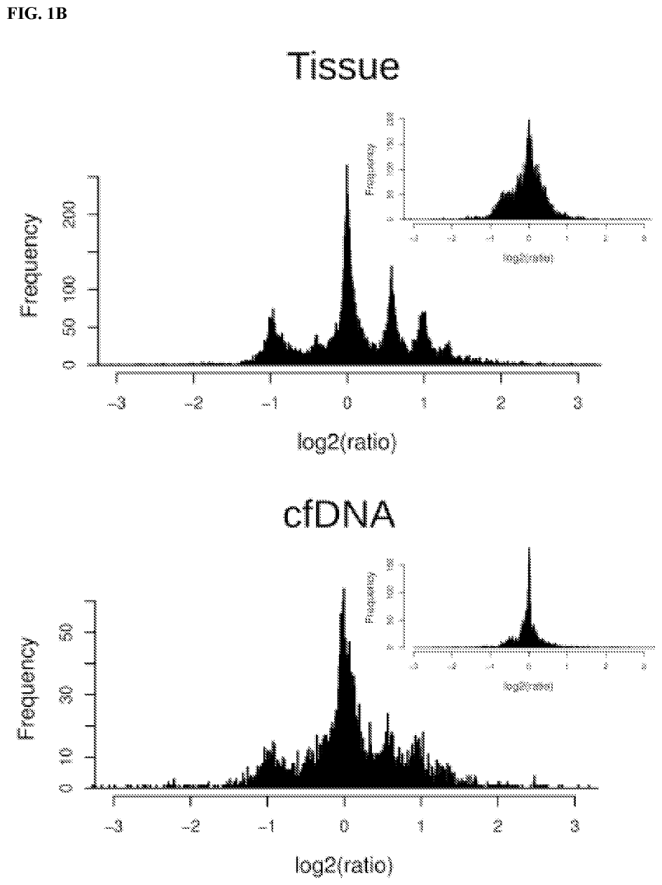

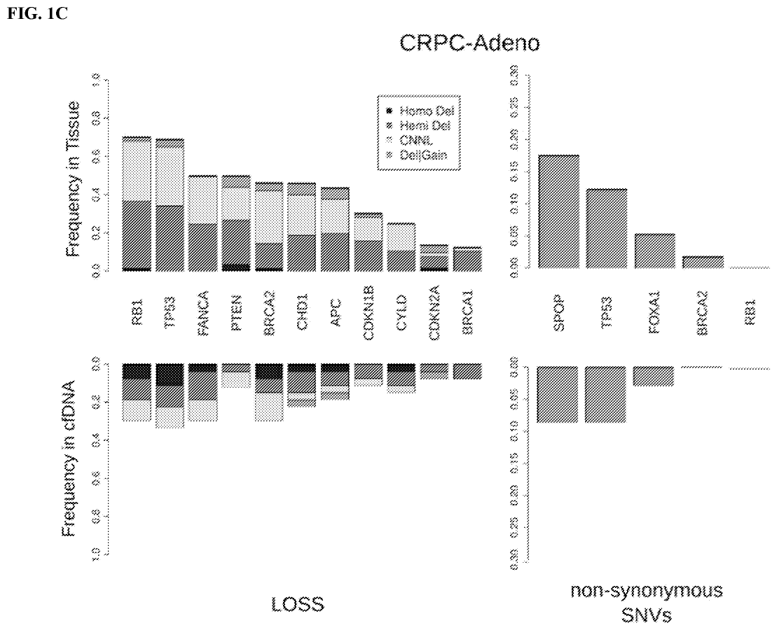

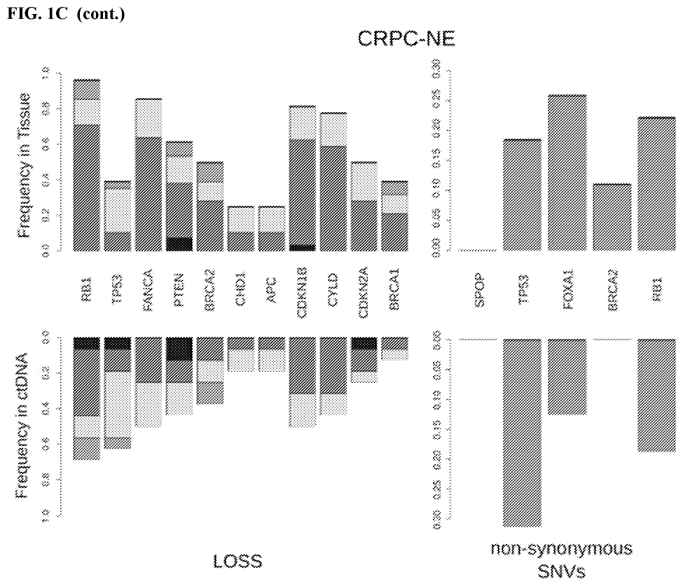

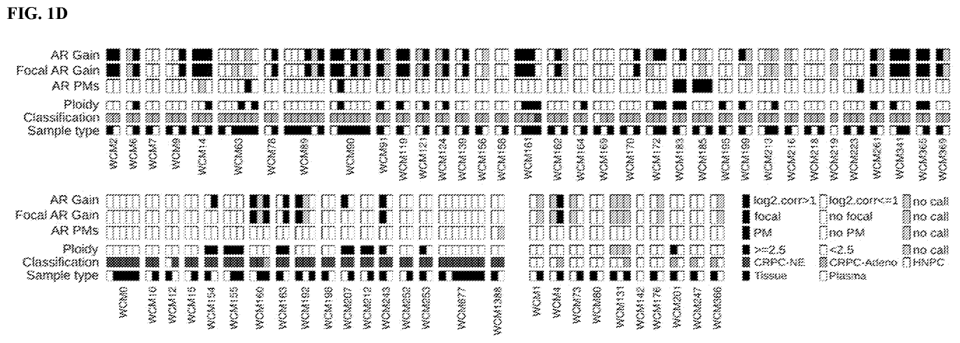

Segmentation of patient's samples was performed using the recent tool FACETS (Shen and Seshan (2016) Nucleic Acids Res 44:e131), a computational method that extends the standard circular binary segmentation (CBS) algorithm to a joint segmentation that combines read counts and SNPs allelic fraction data. To deal with plasma samples, the pre-processing module of FACETS was extended including the de-duplication procedure and segmentation was performed considering separately all three 50th, 75th and 90th percentiles thresholds and complete de-deduplication. Comparison of segmentation results on a large cancer gene list (N=920) showed that although segmentation signal from de-duplicated samples strongly correlates with raw segmentation signal (were no de-duplication was applied) in all cases (FIG. 15E), the stronger the de-duplication level applied the higher the divergence of the signal from the diagonal, indicating a loss of detection power. Based on this observation 75^{th} percentile thresholds was used for cfDNA samples WES segmentation and related somatic copy number downstream analyses (Table 3). Study cohort WES segmented data were adjusted for tumor ploidy and tumor content revealing an overall plasma signal similar to tissue-based data (FIG. 1B), with peaks distinctive of mono- and bi-allelic deletions and of gains of one, two, three or more gene copies.

TABLE 3
Somatic Copy Number Aberration Data

| | Chr | Start | End | log2 | Corrected | | | cnA | cnB |
|---|---|---|---|---|---|---|---|---|---|
| Sample | (hg19) | (hg19) | (hg19) | (ratio) | log2(ratio) | cnA | cnB | integer | integer |
| WCM0 | 1 | 14930 | 11854012 | 0.0028 | −0.1596 | 0.8953 | 0.8953 | 1 | 1 |
| WCM0 | 1 | 11854476 | 19581312 | −0.2629 | −0.7783 | 1.0601 | 0.106 | 1 | 0 |
| WCM0 | 1 | 19582452 | 29823400 | −0.0538 | −0.2795 | 0.8239 | 0.8239 | 1 | 1 |
| WCM0 | 1 | 30117383 | 31214482 | 0.261 | 0.3323 | NA | NA | NA | NA |
| WCM0 | 1 | 31215256 | 50310863 | 0.5154 | 0.7546 | 1.6871 | 1.6871 | 2 | 2 |
| WCM0 | 1 | 50310946 | 91182214 | 0.2759 | 0.3584 | 1.282 | 1.282 | 1 | 1 |
| WCM0 | 1 | 91182531 | 121116688 | −0.317 | −0.9262 | 1.0467 | 0.0058 | 1 | 0 |
| WCM0 | 1 | 142653721 | 248844954 | −0.0199 | −0.2071 | 0.8663 | 0.8663 | 1 | 1 |
| WCM0 | 1 | 248845365 | 249213967 | −0.2354 | −0.7065 | NA | NA | NA | NA |
| WCM0 | 10 | 225982 | 135477913 | −0.3097 | −0.9057 | 1.0509 | 0.0166 | 1 | 0 |
| WCM0 | 11 | 193096 | 433992 | −0.1582 | −0.516 | 1.1376 | 0.261 | 1 | 0 |
| WCM0 | 11 | 434110 | 69482659 | −0.333 | −0.9719 | 1.0275 | −0.0078 | 1 | 0 |
| WCM0 | 11 | 69486539 | 116906728 | 0.1341 | 0.1005 | 1.0721 | 1.0721 | 1 | 1 |
| WCM0 | 11 | 116967847 | 119005075 | −0.2071 | −0.635 | 0.6504 | 0.6375 | 1 | 1 |
| WCM0 | 11 | 119005627 | 134605906 | 0.1505 | 0.1315 | 1.0954 | 1.0954 | 1 | 1 |
| WCM0 | 12 | 148637 | 25679764 | −0.3255 | −0.9504 | 0.9491 | 0.0858 | 1 | 0 |
| WCM0 | 12 | 25699397 | 40963653 | 0.1749 | 0.1769 | 1.1305 | 1.1305 | 1 | 1 |
| WCM0 | 12 | 40964265 | 133810703 | −0.3357 | −0.9798 | 1.0237 | −0.0096 | 1 | 0 |
| WCM0 | 13 | 19042005 | 78335167 | −0.285 | −0.8375 | 1.1206 | −0.0014 | 1 | 0 |
| WCM0 | 13 | 78337334 | 115028400 | 0.2542 | 0.3202 | 2.5145 | −0.0174 | 3 | 0 |
| WCM0 | 13 | 115030714 | 115089411 | −0.0081 | −0.1823 | NA | NA | NA | NA |
| WCM0 | 14 | 19110263 | 107283269 | 0.015 | −0.1344 | 0.9111 | 0.9111 | 1 | 1 |
| WCM0 | 15 | 20083897 | 102506284 | 0.0657 | −0.0322 | 0.9779 | 0.9779 | 1 | 1 |
| WCM0 | 16 | 67600 | 90168697 | −0.322 | −0.9403 | 1.1154 | −0.0731 | 1 | 0 |
| WCM0 | 17 | 131595 | 7592928 | 0.1833 | 0.1923 | 1.1426 | 1.1426 | 1 | 1 |
| WCM0 | 17 | 7604042 | 8094020 | −0.2775 | −0.8172 | 0.7265 | 0.4086 | 1 | 0 |
| WCM0 | 17 | 8094200 | 28772930 | 0.0965 | 0.0283 | 1.0198 | 1.0198 | 1 | 1 |
| WCM0 | 17 | 28776653 | 59760697 | −0.328 | −0.9576 | 1.0713 | −0.0414 | 1 | 0 |
| WCM0 | 17 | 59760996 | 81187901 | 0.1457 | 0.1223 | 1.0885 | 1.0885 | 1 | 1 |
| WCM0 | 18 | 47689 | 15322279 | 0.406 | 0.5789 | 1.4937 | 1.4937 | 1 | 1 |
| WCM0 | 18 | 15323346 | 78005214 | −0.3025 | −0.8856 | 1.058 | 0.0246 | 1 | 0 |
| WCM0 | 19 | 105010 | 7807937 | 0.1688 | 0.1656 | 1.1216 | 1.1216 | 1 | 1 |
| WCM0 | 19 | 7808088 | 9921565 | 0.8084 | 1.1912 | 3.1723 | 1.3945 | 3 | 1 |
| WCM0 | 19 | 9921880 | 10506754 | 0.3014 | 0.4028 | 1.3221 | 1.3221 | 1 | 1 |
| WCM0 | 19 | 10506911 | 12902785 | 0.7952 | 1.1723 | 2.8098 | 1.6976 | 3 | 2 |
| WCM0 | 19 | 12903623 | 17213331 | 0.3231 | 0.4401 | 1.4795 | 1.2339 | 1 | 1 |
| WCM0 | 19 | 17256318 | 19932367 | 0.8172 | 1.2037 | 3.5045 | 1.1021 | 4 | 1 |
| WCM0 | 19 | 19938310 | 37006142 | 0.2043 | 0.2307 | 1.1734 | 1.1734 | 1 | 1 |
| WCM0 | 19 | 37006525 | 38708459 | 0.6666 | 0.9852 | 2.6912 | 1.268 | 3 | 1 |
| WCM0 | 19 | 38709205 | 42731594 | 0.2423 | 0.2992 | 1.2305 | 1.2305 | 1 | 1 |
| WCM0 | 19 | 42734945 | 42852555 | −1.203 | NA | NA | NA | NA | NA |
| WCM0 | 19 | 42853770 | 43883320 | 0.5546 | 0.8156 | 3.485 | 0.035 | 3 | 0 |
| WCM0 | 19 | 43910623 | 44174086 | −0.6559 | −2.2179 | 0.4019 | 0.028 | 0 | 0 |
| WCM0 | 19 | 44222890 | 44335554 | 0.2177 | 0.2551 | NA | NA | NA | NA |
| WCM0 | 19 | 44339735 | 46305466 | 0.6859 | 1.0139 | 2.2041 | 1.8345 | 2 | 2 |
| WCM0 | 19 | 46307593 | 50931526 | 0.1288 | 0.0904 | 1.0646 | 1.0646 | 1 | 1 |
| WCM0 | 19 | 50936235 | 52934785 | 0.6369 | 0.9409 | 2.104 | 1.7353 | 2 | 2 |
| WCM0 | 19 | 52937271 | 59093696 | 0.2368 | 0.2893 | 1.222 | 1.222 | 1 | 1 |
| WCM0 | 2 | 41662 | 69870406 | −0.0445 | −0.2595 | 0.8354 | 0.8354 | 1 | 1 |
| WCM0 | 2 | 69871128 | 88411605 | −0.3345 | −0.9762 | 1.049 | −0.0323 | 1 | 0 |
| WCM0 | 2 | 88422614 | 89035298 | −0.3 | −0.8788 | NA | NA | NA | NA |
| WCM0 | 2 | 89036230 | 113289032 | 0.038 | −0.0875 | 0.9412 | 0.9412 | 1 | 1 |
| WCM0 | 2 | 113300115 | 149449371 | −0.2819 | −0.8291 | 1.0002 | 0.1256 | 1 | 0 |
| WCM0 | 2 | 149501317 | 228135631 | 0.1574 | 0.1443 | 1.1052 | 1.1052 | 1 | 1 |
| WCM0 | 2 | 228135709 | 230253000 | −0.0446 | −0.2598 | 0.8352 | 0.8352 | 1 | 1 |
| WCM0 | 2 | 230271992 | 234368816 | −0.2861 | −0.8405 | 0.9839 | 0.1331 | 1 | 0 |
| WCM0 | 2 | 234368850 | 243037091 | 0.0815 | −0.001 | 0.9993 | 0.9993 | 1 | 1 |
| WCM0 | 20 | 68396 | 62934572 | 0.0544 | −0.0545 | 0.9629 | 0.9629 | 1 | 1 |
| WCM0 | 21 | 9590305 | 48084175 | 0.1104 | 0.0551 | 1.0389 | 1.0389 | 1 | 1 |
| WCM0 | 22 | 16159060 | 51219089 | −0.3237 | −0.9453 | 1.0299 | 0.0087 | 1 | 0 |
| WCM0 | 3 | 239472 | 73657753 | −0.3311 | −0.9665 | 1.036 | −0.0125 | 1 | 0 |
| WCM0 | 3 | 73673872 | 94226102 | 0.1212 | 0.076 | 1.0541 | 1.0541 | 1 | 1 |
| WCM0 | 3 | 94226357 | 122143416 | −0.2615 | −0.7746 | 1.1177 | 0.0514 | 1 | 0 |
| WCM0 | 3 | 122143751 | 197896656 | 0.1542 | 0.1384 | 1.1007 | 1.1007 | 1 | 1 |
| WCM0 | 4 | 53397 | 61529776 | −0.0769 | −0.33 | 0.7955 | 0.7955 | 1 | 1 |
| WCM0 | 4 | 62066964 | 175429233 | 0.1502 | 0.1308 | 1.0949 | 1.0949 | 1 | 1 |
| WCM0 | 4 | 175429954 | 190878508 | −0.025 | −0.2178 | 0.8599 | 0.8599 | 1 | 1 |
| WCM0 | 5 | 90714 | 123972927 | 0.0519 | −0.0595 | 0.9596 | 0.9596 | 1 | 1 |
| WCM0 | 5 | 123973546 | 136953689 | −0.3301 | −0.9637 | 1.0388 | −0.0133 | 1 | 0 |
| WCM0 | 5 | 136953959 | 180687824 | 0.1227 | 0.0787 | 1.056 | 1.056 | 1 | 1 |
| WCM0 | 6 | 203546 | 126202321 | 0.1298 | 0.0923 | 1.0661 | 1.0661 | 1 | 1 |
| WCM0 | 6 | 126203596 | 166743676 | −0.0652 | −0.3044 | 0.8098 | 0.8098 | 1 | 1 |
| WCM0 | 6 | 166744943 | 170892921 | 0.1034 | 0.0417 | 1.0294 | 1.0294 | 1 | 1 |
| WCM0 | 7 | 193245 | 29519994 | 0.1331 | 0.0985 | 1.0707 | 1.0707 | 1 | 1 |
| WCM0 | 7 | 29523654 | 38269668 | −0.3354 | −0.9787 | 0.9773 | 0.0376 | 1 | 0 |
| WCM0 | 7 | 38269935 | 158937074 | 0.1203 | 0.0742 | 1.0527 | 1.0527 | 1 | 1 |
| WCM0 | 8 | 163457 | 2092866 | −1.0648 | NA | −0.0753 | −0.0753 | 0 | 0 |
| WCM0 | 8 | 2795340 | 36703319 | −0.1837 | −0.5773 | 1.2818 | 0.0586 | 1 | 0 |
| WCM0 | 8 | 36704395 | 105544113 | 0.2049 | 0.2319 | 1.1744 | 1.1744 | 1 | 1 |
| WCM0 | 8 | 105601136 | 146279398 | −0.201 | −0.6198 | 1.268 | 0.0335 | 1 | 0 |
| WCM0 | 9 | 14876 | 3469140 | −0.6076 | −1.9718 | 0.3194 | 0.1905 | 0 | 0 |
| WCM0 | 9 | 3824162 | 19032837 | −0.0113 | −0.189 | 0.8772 | 0.8772 | 1 | 1 |
| WCM0 | 9 | 19049691 | 37735766 | −0.2567 | −0.7619 | 1.1867 | −0.0072 | 1 | 0 |
| WCM0 | 9 | 37737138 | 97321156 | −0.0346 | −0.2383 | 0.8478 | 0.8478 | 1 | 1 |
| WCM0 | 9 | 97321430 | 141107525 | 0.1381 | 0.1079 | 1.0777 | 1.0777 | 1 | 1 |
| WCM0 | X | 2700082 | 123026595 | 0.0914 | 0.0183 | NA | NA | NA | NA |
| WCM0 | X | 123034363 | 154493590 | 0.361 | 0.5041 | NA | NA | NA | NA |
| WCM0_LM1 | 1 | 809022 | 2560735 | −0.2824 | −0.5322 | 0.917 | 0.466 | 1 | 0 |
| WCM0_LM1 | 1 | 2938318 | 6215836 | −0.1019 | −0.3389 | 1.031 | 0.5503 | 1 | 1 |
| WCM0_LM1 | 1 | 6219610 | 11986007 | −0.3283 | −0.5817 | 0.883 | 0.4534 | 1 | 0 |
| WCM0_LM1 | 1 | 11994910 | 19609254 | −0.8045 | −1.103 | 0.9417 | −0.0107 | 1 | 0 |
| WCM0_LM1 | 1 | 19611241 | 26441091 | −0.2779 | −0.5273 | 0.9148 | 0.4729 | 1 | 0 |
| WCM0_LM1 | 1 | 26448447 | 29391691 | −0.4169 | −0.6775 | 0.8327 | 0.4178 | 1 | 0 |
| WCM0_LM1 | 1 | 29424270 | 29652174 | −0.1691 | −0.4107 | NA | NA | NA | NA |
| WCM0_LM1 | 1 | 31186472 | 31899691 | 0.7196 | 0.5237 | NA | NA | NA | NA |
| WCM0_LM1 | 1 | 31901898 | 32265645 | 0.941 | 0.7526 | NA | NA | NA | NA |
| WCM0_LM1 | 1 | 32266186 | 33613323 | 0.668 | 0.4702 | 1.9677 | 0.803 | 2 | 1 |
| WCM0_LM1 | 1 | 33623891 | 35370589 | 1.1411 | 0.9585 | NA | NA | NA | NA |
| WCM0_LM1 | 1 | 35370948 | 41300682 | 0.7621 | 0.5677 | 2.0562 | 0.9082 | 2 | 1 |
| WCM0_LM1 | 1 | 41303372 | 44778928 | 0.9534 | 0.7654 | 2.3307 | 1.0689 | 2 | 1 |
| WCM0_LM1 | 1 | 44785393 | 50886915 | 0.8126 | 0.62 | 2.1425 | 0.9313 | 2 | 1 |
| WCM0_LM1 | 1 | 50907098 | 55203321 | 0.3774 | 0.1673 | 1.3052 | 0.9408 | 1 | 1 |
| WCM0_LM1 | 1 | 55207079 | 61920966 | 0.6683 | 0.4705 | 1.6157 | 1.1555 | 2 | 1 |
| WCM0_LM1 | 1 | 62149156 | 64011693 | 0.346 | 0.1344 | 1.3035 | 0.8917 | 1 | 1 |
| WCM0_LM1 | 1 | 64017391 | 91182777 | 0.5953 | 0.3947 | 1.502 | 1.1274 | 2 | 1 |
| WCM0_LM1 | 1 | 91382484 | 92200462 | −0.6736 | −0.9581 | NA | NA | NA | NA |
| WCM0_LM1 | 1 | 92224347 | 94012976 | −0.8216 | −1.122 | 0.9474 | −0.0286 | 1 | 0 |
| WCM0_LM1 | 1 | 94014931 | 109154756 | −0.5479 | −0.8201 | 1.1279 | 0.0049 | 1 | 0 |
| WCM0_LM1 | 1 | 109158979 | 109968887 | −0.792 | −1.0891 | 0.9572 | −0.0171 | 1 | 0 |
| WCM0_LM1 | 1 | 110009487 | 112525389 | −0.5036 | −0.7717 | 1.1653 | 0.0061 | 1 | 0 |
| WCM0_LM1 | 1 | 112958891 | 115469166 | −0.7305 | −1.0209 | 0.9957 | −0.0101 | 1 | 0 |
| WCM0_LM1 | 1 | 115487092 | 120166453 | −0.4917 | −0.7588 | 1.1831 | −0.0011 | 1 | 0 |
| WCM0_LM1 | 1 | 120166737 | 120612013 | −0.3588 | −0.6146 | NA | NA | NA | NA |
| WCM0_LM1 | 1 | 120612265 | 149943111 | −0.0077 | −0.2386 | 1.0096 | 0.6855 | 1 | 1 |
| WCM0_LM1 | 1 | 149949406 | 151824941 | −0.2546 | −0.5023 | 0.8038 | 0.6081 | 1 | 1 |
| WCM0_LM1 | 1 | 151825991 | 153517315 | 0.0987 | −0.1258 | 1.2528 | 0.5802 | 1 | 1 |
| WCM0_LM1 | 1 | 153520298 | 156846393 | −0.3847 | −0.6427 | 0.8457 | 0.4354 | 1 | 0 |
| WCM0_LM1 | 1 | 156848995 | 160846646 | −0.0056 | −0.2364 | 1.1004 | 0.5973 | 1 | 1 |
| WCM0_LM1 | 1 | 160849077 | 161487946 | −0.3753 | −0.6324 | NA | NA | NA | NA |
| WCM0_LM1 | 1 | 161494809 | 180461504 | −0.0819 | −0.3175 | 1.0393 | 0.5656 | 1 | 1 |
| WCM0_LM1 | 1 | 180464581 | 198828261 | 0.2065 | −0.0121 | 0.9979 | 0.9854 | 1 | 1 |
| WCM0_LM1 | 1 | 199997031 | 205739877 | 0.0392 | −0.1889 | 0.9591 | 0.7954 | 1 | 1 |
| WCM0_LM1 | 1 | 205740573 | 211433487 | 0.2414 | 0.0247 | 1.0718 | 0.9627 | 1 | 1 |
| WCM0_LM1 | 1 | 211444578 | 213414090 | −0.0266 | −0.2587 | 0.8589 | 0.8127 | 1 | 1 |
| WCM0_LM1 | 1 | 213414438 | 223957621 | 0.1938 | −0.0255 | 0.997 | 0.968 | 1 | 1 |
| WCM0_LM1 | 1 | 223958181 | 226568728 | −0.0687 | −0.3034 | 0.844 | 0.7767 | 1 | 1 |
| WCM0_LM1 | 1 | 226570771 | 233231579 | 0.1508 | −0.0708 | 1.0335 | 0.8707 | 1 | 1 |
| WCM0_LM1 | 1 | 233270873 | 233394468 | −0.5374 | −0.8086 | NA | NA | NA | NA |
| WCM0_LM1 | 1 | 233394739 | 234745259 | 0.1789 | −0.0412 | 1.0886 | 0.855 | 1 | 1 |
| WCM0_LM1 | 1 | 235277252 | 236766527 | −0.1217 | −0.36 | 0.8241 | 0.7343 | 1 | 1 |
| WCM0_LM1 | 1 | 236767326 | 246493813 | 0.1125 | −0.1113 | 1.0226 | 0.8289 | 1 | 1 |
| WCM0_LM1 | 1 | 246498713 | 247582128 | −0.0918 | −0.3281 | NA | NA | NA | NA |
| WCM0_LM1 | 1 | 247582351 | 249212569 | 0.2842 | 0.0696 | 1.1905 | 0.9084 | 1 | 1 |
| WCM0_LM1 | 10 | 226016 | 5435752 | −0.5291 | −0.7996 | 1.1446 | 0.0044 | 1 | 0 |
| WCM0_LM1 | 10 | 5436261 | 44873331 | −0.7452 | −1.0371 | 0.9786 | −0.004 | 1 | 0 |
| WCM0_LM1 | 10 | 44873732 | 69106478 | −0.52 | −0.7896 | 1.1528 | 0.0042 | 1 | 0 |
| WCM0_LM1 | 10 | 69281647 | 71139853 | −1.028 | −1.3541 | 0.8048 | −0.0225 | 1 | 0 |
| WCM0_LM1 | 10 | 71142230 | 73857254 | −0.6241 | −0.9036 | 1.0607 | 0.0084 | 1 | 0 |
| WCM0_LM1 | 10 | 73887823 | 76982173 | −0.8501 | −1.1539 | 0.9108 | −0.012 | 1 | 0 |
| WCM0_LM1 | 10 | 76989390 | 93582099 | −0.5882 | −0.8642 | 1.0971 | 0.0015 | 1 | 0 |
| WCM0_LM1 | 10 | 93586976 | 99511158 | −0.7617 | −1.0555 | 0.9669 | −0.0046 | 1 | 0 |
| WCM0_LM1 | 10 | 99512581 | 101162498 | −0.5082 | −0.7768 | 1.1735 | −0.0062 | 1 | 0 |
| WCM0_LM1 | 10 | 101163194 | 105206915 | −0.8467 | −1.1501 | 0.9115 | −0.0104 | 1 | 0 |
| WCM0_LM1 | 10 | 105207126 | 120460820 | −0.6083 | −0.8862 | 1.0801 | 0.002 | 1 | 0 |
| WCM0_LM1 | 10 | 120467037 | 121680507 | −0.8483 | −1.1518 | 0.908 | −0.0079 | 1 | 0 |
| WCM0_LM1 | 10 | 121685686 | 135373640 | −0.5776 | −0.8526 | 1.1056 | 0.0019 | 1 | 0 |
| WCM0_LM1 | 11 | 193096 | 1776250 | −0.7824 | −1.0785 | 0.9408 | 0.0063 | 1 | 0 |
| WCM0_LM1 | 11 | 1776299 | 4389395 | −0.6441 | −0.9255 | 1.0697 | −0.0167 | 1 | 0 |
| WCM0_LM1 | 11 | 4406727 | 8123157 | −0.4237 | −0.6848 | 1.2361 | 0.0081 | 1 | 0 |
| WCM0_LM1 | 11 | 8132292 | 8959697 | −0.5917 | −0.868 | 1.1034 | −0.0076 | 1 | 0 |
| WCM0_LM1 | 11 | 8970045 | 10064480 | −0.8553 | −1.1597 | 0.9078 | −0.0126 | 1 | 0 |
| WCM0_LM1 | 11 | 10215446 | 16824514 | −0.4859 | −0.7525 | 1.1823 | 0.0049 | 1 | 0 |
| WCM0_LM1 | 11 | 16824776 | 18787411 | −0.6992 | −0.9863 | 1.0215 | −0.0119 | 1 | 0 |
| WCM0_LM1 | 11 | 18793550 | 32622213 | −0.4574 | −0.7215 | 1.2076 | 0.0054 | 1 | 0 |
| WCM0_LM1 | 11 | 32623301 | 33375034 | −0.8244 | −1.1252 | NA | NA | NA | NA |
| WCM0_LM1 | 11 | 33564085 | 45970423 | −0.5422 | −0.8139 | 1.1251 | 0.0126 | 1 | 0 |
| WCM0_LM1 | 11 | 45970983 | 48185007 | −0.8971 | −1.2064 | 0.8814 | −0.0147 | 1 | 0 |
| WCM0_LM1 | 11 | 48186104 | 61026319 | −0.5808 | −0.8561 | 1.0903 | 0.0145 | 1 | 0 |
| WCM0_LM1 | 11 | 61026496 | 69488069 | −0.8041 | −1.1025 | 0.9374 | −0.006 | 1 | 0 |
| WCM0_LM1 | 11 | 69490017 | 78239950 | 0.2413 | 0.0245 | 1.0214 | 1.0128 | 1 | 1 |
| WCM0_LM1 | 11 | 78270557 | 107224313 | 0.3748 | 0.1646 | 1.1267 | 1.115 | 1 | 1 |
| WCM0_LM1 | 11 | 107260927 | 107462599 | 0.2867 | 0.0723 | NA | NA | NA | NA |
| WCM0_LM1 | 11 | 107463154 | 108351984 | 0.0341 | −0.1943 | 0.8935 | 0.8546 | 1 | 1 |
| WCM0_LM1 | 11 | 108352910 | 116824749 | 0.2824 | 0.0678 | 1.0877 | 1.0085 | 1 | 1 |
| WCM0_LM1 | 11 | 116827649 | 117150646 | −0.835 | −1.137 | NA | NA | NA | NA |
| WCM0_LM1 | 11 | 117150988 | 118656889 | −0.164 | −0.4052 | 0.9967 | 0.5136 | 1 | 1 |
| WCM0_LM1 | 11 | 118754661 | 119025220 | −0.8446 | −1.1477 | NA | NA | NA | NA |
| WCM0_LM1 | 11 | 119025569 | 134257523 | 0.3485 | 0.1371 | 1.1054 | 1.0939 | 1 | 1 |
| WCM0_LM1 | 12 | 247582 | 6425588 | −0.6186 | −0.8975 | 1.0726 | 0.001 | 1 | 0 |
| WCM0_LM1 | 12 | 6426664 | 7069357 | −0.8563 | −1.1608 | 0.9102 | −0.0156 | 1 | 0 |
| WCM0_LM1 | 12 | 7069584 | 9577125 | −0.6833 | −0.9687 | 1.0261 | −0.0042 | 1 | 0 |
| WCM0_LM1 | 12 | 9578130 | 9747907 | 0.2345 | 0.0174 | NA | NA | NA | NA |
| WCM0_LM1 | 12 | 9750669 | 12874086 | −0.631 | −0.9112 | 1.0565 | 0.007 | 1 | 0 |
| WCM0_LM1 | 12 | 12917601 | 18242237 | −0.263 | −0.5114 | 1.3961 | 0.0069 | 1 | 0 |
| WCM0_LM1 | 12 | 18435035 | 22837810 | −0.8009 | −1.099 | 0.9261 | 0.0076 | 1 | 0 |
| WCM0_LM1 | 12 | 23687369 | 24365375 | −0.1651 | −0.4064 | NA | NA | NA | NA |
| WCM0_LM1 | 12 | 24736749 | 25348029 | −0.9242 | −1.2369 | NA | NA | NA | NA |
| WCM0_LM1 | 12 | 25356961 | 28605426 | 0.1452 | −0.0767 | 0.9762 | 0.9202 | 1 | 1 |
| WCM0_LM1 | 12 | 28637090 | 31359073 | 0.5247 | 0.3212 | 1.4004 | 1.0984 | 1 | 1 |
| WCM0_LM1 | 12 | 31435823 | 33049598 | 0.1572 | −0.0641 | 1.1039 | 0.8092 | 1 | 1 |
| WCM0_LM1 | 12 | 33529782 | 40761617 | 0.5696 | 0.368 | NA | NA | NA | NA |
| WCM0_LM1 | 12 | 41302223 | 41903648 | −0.1107 | −0.3482 | NA | NA | NA | NA |
| WCM0_LM1 | 12 | 41946539 | 49048731 | −0.2021 | −0.446 | 1.4657 | 0.0024 | 1 | 0 |
| WCM0_LM1 | 12 | 49050561 | 51901248 | −0.5135 | −0.7825 | 1.1767 | −0.014 | 1 | 0 |
| WCM0_LM1 | 12 | 52056612 | 53228012 | −0.19 | −0.433 | 1.4698 | 0.0116 | 1 | 0 |
| WCM0_LM1 | 12 | 53233021 | 54070021 | −0.5642 | −0.8379 | 1.1446 | −0.0257 | 1 | 0 |
| WCM0_LM1 | 12 | 54105893 | 56137502 | −0.2827 | −0.5326 | NA | NA | NA | NA |
| WCM0_LM1 | 12 | 56142337 | 57109792 | −0.5773 | −0.8523 | NA | NA | NA | NA |
| WCM0_LM1 | 12 | 57109935 | 57976312 | −0.3855 | −0.6435 | 1.2886 | −0.0083 | 1 | 0 |
| WCM0_LM1 | 12 | 57976887 | 69995124 | −0.3322 | −0.5859 | 1.3327 | −2.00E−04 | 1 | 0 |
| WCM0_LM1 | 12 | 70003852 | 93169842 | −0.1897 | −0.4327 | 1.4709 | 0.0109 | 1 | 0 |
| WCM0_LM1 | 12 | 93170629 | 102455041 | −0.4182 | −0.6789 | 1.2525 | −0.0032 | 1 | 0 |
| WCM0_LM1 | 12 | 102455729 | 108926064 | −0.2438 | −0.4907 | 1.4363 | −0.0129 | 1 | 0 |
| WCM0_LM1 | 12 | 108929226 | 110207078 | −0.4205 | −0.6814 | 1.2544 | −0.0073 | 1 | 0 |
| WCM0_LM1 | 12 | 110221534 | 112924336 | −0.6313 | −0.9115 | NA | NA | NA | NA |
| WCM0_LM1 | 12 | 112926890 | 120546214 | −0.4116 | −0.6718 | 1.2659 | −0.0104 | 1 | 0 |
| WCM0_LM1 | 12 | 120565808 | 124333351 | −0.6187 | −0.8976 | 1.0908 | −0.0173 | 1 | 0 |
| WCM0_LM1 | 12 | 124335464 | 133779375 | −0.3783 | −0.6357 | 1.2912 | −0.0039 | 1 | 0 |
| WCM0_LM1 | 13 | 19705463 | 23869639 | −0.8361 | −1.1382 | 0.9253 | −0.0166 | 1 | 0 |
| WCM0_LM1 | 13 | 23894941 | 61989243 | −0.6127 | −0.891 | 1.0778 | 6.00E−04 | 1 | 0 |
| WCM0_LM1 | 13 | 63745813 | 72014784 | −1.1874 | −1.5364 | NA | NA | NA | NA |
| WCM0_LM1 | 13 | 72049242 | 80911823 | −0.1039 | −0.3411 | 1.5777 | 0.0013 | 2 | 0 |
| WCM0_LM1 | 13 | 84453543 | 115089411 | 0.6286 | 0.4292 | 2.6897 | 0.0033 | 3 | 0 |
| WCM0_LM1 | 14 | 19553559 | 22237284 | 0.0739 | −0.1521 | 0.9057 | 0.8942 | 1 | 1 |
| WCM0_LM1 | 14 | 22251213 | 23012177 | 0.3652 | 0.1546 | 1.1329 | 1.0933 | 1 | 1 |
| WCM0_LM1 | 14 | 23013081 | 23652157 | −0.0473 | −0.2808 | 0.8608 | 0.7855 | 1 | 1 |
| WCM0_LM1 | 14 | 23745164 | 31348712 | 0.1963 | −0.0229 | 0.9967 | 0.9718 | 1 | 1 |
| WCM0_LM1 | 14 | 31349614 | 34419896 | 0.0188 | −0.2105 | 0.9119 | 0.8165 | 1 | 1 |
| WCM0_LM1 | 14 | 34904506 | 35871262 | −0.2295 | −0.4754 | NA | NA | NA | NA |
| WCM0_LM1 | 14 | 35871728 | 50052772 | 0.1212 | −0.1021 | 0.9445 | 0.9189 | 1 | 1 |
| WCM0_LM1 | 14 | 50065793 | 51102099 | −0.0642 | −0.2987 | NA | NA | NA | NA |
| WCM0_LM1 | 14 | 51107548 | 73198645 | 0.1367 | −0.0857 | 0.9473 | 0.9373 | 1 | 1 |
| WCM0_LM1 | 14 | 73220020 | 75904831 | −0.0624 | −0.2968 | 0.8574 | 0.7707 | 1 | 1 |
| WCM0_LM1 | 14 | 75928152 | 93994954 | 0.0887 | −0.1365 | 0.9213 | 0.8982 | 1 | 1 |
| WCM0_LM1 | 14 | 93995602 | 95236210 | 0.2904 | 0.0761 | 1.0663 | 1.042 | 1 | 1 |
| WCM0_LM1 | 14 | 95236360 | 95599836 | −0.7817 | −1.0777 | NA | NA | NA | NA |
| WCM0_LM1 | 14 | 95658021 | 102228255 | 0.2342 | 0.0171 | 1.1045 | 0.9193 | 1 | 1 |
| WCM0_LM1 | 14 | 102229325 | 107283105 | 0.0268 | −0.202 | 0.932 | 0.8066 | 1 | 1 |
| WCM0_LM1 | 15 | 20170006 | 40846181 | 0.2471 | 0.0306 | 1.1138 | 0.9291 | 1 | 1 |
| WCM0_LM1 | 15 | 40849475 | 42058895 | −0.0847 | −0.3205 | NA | NA | NA | NA |
| WCM0_LM1 | 15 | 42058998 | 42500371 | 0.2532 | 0.0371 | 1.0562 | 0.9958 | 1 | 1 |
| WCM0_LM1 | 15 | 42503944 | 45059787 | 0.0633 | −0.1633 | NA | NA | NA | NA |
| WCM0_LM1 | 15 | 45059972 | 45981276 | 0.1966 | −0.0225 | NA | NA | NA | NA |
| WCM0_LM1 | 15 | 45983210 | 50489713 | 0.4166 | 0.2083 | 1.188 | 1.1227 | 1 | 1 |
| WCM0_LM1 | 15 | 50489902 | 50546748 | 0.2013 | −0.0175 | NA | NA | NA | NA |
| WCM0_LM1 | 15 | 50546876 | 51216171 | −0.1414 | −0.381 | NA | NA | NA | NA |
| WCM0_LM1 | 15 | 51217361 | 64367657 | 0.2173 | −7.00E−04 | 1.0027 | 0.9963 | 1 | 1 |
| WCM0_LM1 | 15 | 64373313 | 66853399 | −0.0188 | −0.2505 | NA | NA | NA | NA |
| WCM0_LM1 | 15 | 66853613 | 74635515 | 0.2342 | 0.0171 | 1.0193 | 1.0044 | 1 | 1 |
| WCM0_LM1 | 15 | 74636126 | 76183257 | 0.041 | −0.187 | 0.8961 | 0.8608 | 1 | 1 |
| WCM0_LM1 | 15 | 76189333 | 90173735 | 0.236 | 0.0189 | 1.0175 | 1.009 | 1 | 1 |
| WCM0_LM1 | 15 | 90174824 | 91548893 | 0.0333 | −0.1951 | 0.8763 | 0.8707 | 1 | 1 |
| WCM0_LM1 | 15 | 91549254 | 102359167 | 0.2896 | 0.0752 | 1.1299 | 0.9772 | 1 | 1 |
| WCM0_LM1 | 16 | 79642 | 3063747 | −0.801 | −1.0992 | 0.9394 | −0.0059 | 1 | 0 |
| WCM0_LM1 | 16 | 3064029 | 19711751 | −0.8781 | −1.1851 | 0.8879 | −0.0083 | 1 | 0 |
| WCM0_LM1 | 16 | 19718309 | 20990708 | −0.6633 | −0.9467 | 1.0422 | −0.0046 | 1 | 0 |
| WCM0_LM1 | 16 | 20994031 | 31202744 | −0.9154 | −1.2271 | 0.8681 | −0.0138 | 1 | 0 |
| WCM0_LM1 | 16 | 31212936 | 54317607 | −0.6781 | −0.9631 | 1.0463 | −0.0204 | 1 | 0 |
| WCM0_LM1 | 16 | 54318237 | 56435736 | −0.3991 | −0.6582 | NA | NA | NA | NA |
| WCM0_LM1 | 16 | 56437057 | 66638885 | −0.7449 | −1.0369 | 0.9826 | −0.0078 | 1 | 0 |
| WCM0_LM1 | 16 | 66642326 | 70710859 | −0.8779 | −1.185 | 0.8919 | −0.0122 | 1 | 0 |
| WCM0_LM1 | 16 | 70711784 | 90162300 | −0.7306 | −1.021 | 0.994 | −0.0085 | 1 | 0 |
| WCM0_LM1 | 17 | 131595 | 5392711 | 0.0242 | −0.2047 | 0.8787 | 0.8567 | 1 | 1 |
| WCM0_LM1 | 17 | 5405186 | 7579619 | 0.1419 | −0.0802 | 0.9529 | 0.9389 | 1 | 1 |
| WCM0_LM1 | 17 | 7579885 | 7951724 | −0.7752 | −1.0705 | NA | NA | NA | NA |
| WCM0_LM1 | 17 | 7976291 | 8647931 | −0.3035 | −0.555 | 0.9065 | 0.4548 | 1 | 0 |
| WCM0_LM1 | 17 | 8700863 | 28030079 | 0.1612 | −0.0598 | 1.0231 | 0.8956 | 1 | 1 |
| WCM0_LM1 | 17 | 28120949 | 28773023 | 0.7014 | 0.5049 | NA | NA | NA | NA |
| WCM0_LM1 | 17 | 28776598 | 30807591 | −0.8739 | −1.1805 | 0.902 | −0.0195 | 1 | 0 |
| WCM0_LM1 | 17 | 30814851 | 35482636 | −0.5999 | −0.877 | 1.095 | −0.0061 | 1 | 0 |
| WCM0_LM1 | 17 | 35486267 | 38640824 | −0.838 | −1.1404 | 0.928 | −0.0207 | 1 | 0 |
| WCM0_LM1 | 17 | 38641202 | 39728021 | −0.5432 | −0.8149 | 1.1404 | −0.0036 | 1 | 0 |
| WCM0_LM1 | 17 | 39728267 | 47899172 | −0.8254 | −1.1263 | 0.9274 | −0.0113 | 1 | 0 |
| WCM0_LM1 | 17 | 47903535 | 56492800 | −0.6044 | −0.8819 | 1.0957 | −0.0104 | 1 | 0 |
| WCM0_LM1 | 17 | 56492857 | 59857788 | −0.8112 | −1.1105 | 0.9377 | −0.0114 | 1 | 0 |
| WCM0_LM1 | 17 | 59858241 | 60743932 | −0.0055 | −0.2363 | NA | NA | NA | NA |
| WCM0_LM1 | 17 | 60744176 | 65105587 | 0.1986 | −0.0204 | 0.9974 | 0.9745 | 1 | 1 |
| WCM0_LM1 | 17 | 65105774 | 66526145 | −0.0504 | −0.284 | 0.8493 | 0.7934 | 1 | 1 |
| WCM0_LM1 | 17 | 66526506 | 70644905 | 0.3502 | 0.1389 | 1.1184 | 1.0837 | 1 | 1 |
| WCM0_LM1 | 17 | 70645306 | 72921026 | 0.1554 | −0.0659 | 0.9614 | 0.9493 | 1 | 1 |
| WCM0_LM1 | 17 | 72921656 | 76853714 | 0.0538 | −0.1734 | 0.93 | 0.8435 | 1 | 1 |
| WCM0_LM1 | 17 | 76867017 | 81052308 | 0.1868 | −0.0328 | 0.9919 | 0.9631 | 1 | 1 |
| WCM0_LM1 | 18 | 47653 | 14613577 | 0.7548 | 0.5602 | 1.9907 | 0.9583 | 2 | 1 |
| WCM0_LM1 | 18 | 18539857 | 21242720 | −0.9097 | −1.2206 | NA | NA | NA | NA |
| WCM0_LM1 | 18 | 21269700 | 74980821 | −0.5838 | −0.8594 | 1.1007 | 0.0017 | 1 | 0 |
| WCM0_LM1 | 18 | 76753131 | 77287601 | 0.8548 | 0.6636 | NA | NA | NA | NA |
| WCM0_LM1 | 18 | 77440043 | 78005210 | −0.4837 | −0.75 | NA | NA | NA | NA |
| WCM0_LM1 | 19 | 281360 | 498098 | 0.2575 | 0.0416 | NA | NA | NA | NA |
| WCM0_LM1 | 19 | 498520 | 6366363 | −0.0514 | −0.2851 | 0.8827 | 0.7587 | 1 | 1 |
| WCM0_LM1 | 19 | 6368657 | 6988812 | 0.2752 | 0.0601 | 1.266 | 0.8191 | 1 | 1 |
| WCM0_LM1 | 19 | 6990781 | 7512016 | 0.929 | 0.7403 | NA | NA | NA | NA |
| WCM0_LM1 | 19 | 7516176 | 7810674 | 0.2416 | 0.0249 | 1.256 | 0.7788 | 1 | 1 |
| WCM0_LM1 | 19 | 7810937 | 8203067 | 1.007 | 0.8206 | 2.7301 | 0.8022 | 3 | 1 |
| WCM0_LM1 | 19 | 8203328 | 8326901 | 0.8551 | 0.6639 | NA | NA | NA | NA |
| WCM0_LM1 | 19 | 8367464 | 8491707 | 0.2259 | 0.0083 | 1.2482 | 0.7634 | 1 | 1 |
| WCM0_LM1 | 19 | 8495640 | 9771348 | 1.0262 | 0.8404 | 2.7379 | 0.8432 | 3 | 1 |
| WCM0_LM1 | 19 | 9801332 | 10504113 | 0.1953 | −0.0239 | 1.1421 | 0.825 | 1 | 1 |
| WCM0_LM1 | 19 | 10505871 | 10692397 | 0.9094 | 0.72 | NA | NA | NA | NA |
| WCM0_LM1 | 19 | 10694339 | 10829001 | 1.4212 | 1.2455 | NA | NA | NA | NA |
| WCM0_LM1 | 19 | 10870441 | 10939946 | 0.8927 | 0.7028 | NA | NA | NA | NA |
| WCM0_LM1 | 19 | 10940882 | 11326489 | 1.4646 | 1.2899 | 4.171 | 0.7192 | 4 | 1 |
| WCM0_LM1 | 19 | 11327571 | 11348394 | 0.9916 | 0.8048 | NA | NA | NA | NA |
| WCM0_LM1 | 19 | 11348911 | 11408997 | 0.9161 | 0.727 | NA | NA | NA | NA |
| WCM0_LM1 | 19 | 11409571 | 11598603 | 0.2027 | −0.0161 | NA | NA | NA | NA |
| WCM0_LM1 | 19 | 11606831 | 12089920 | 1.4349 | 1.2596 | NA | NA | NA | NA |
| WCM0_LM1 | 19 | 12125850 | 12577572 | 0.2036 | −0.0152 | NA | NA | NA | NA |
| WCM0_LM1 | 19 | 12595529 | 12886276 | 0.9556 | 0.7677 | NA | NA | NA | NA |
| WCM0_LM1 | 19 | 12902681 | 14703188 | 0.2622 | 0.0465 | 1.2107 | 0.8547 | 1 | 1 |
| WCM0_LM1 | 19 | 14705319 | 15221074 | 1.0422 | 0.8568 | 2.7988 | 0.8233 | 3 | 1 |
| WCM0_LM1 | 19 | 15221533 | 15795976 | 0.392 | 0.1826 | 1.3605 | 0.9093 | 1 | 1 |
| WCM0_LM1 | 19 | 15806710 | 15882828 | 0.5559 | 0.3537 | NA | NA | NA | NA |
| WCM0_LM1 | 19 | 15905002 | 16222893 | 1.0801 | 0.8958 | 2.8671 | 0.8543 | 3 | 1 |
| WCM0_LM1 | 19 | 16229016 | 16436803 | 0.2968 | 0.0828 | NA | NA | NA | NA |
| WCM0_LM1 | 19 | 16437752 | 16548596 | 1.005 | 0.8185 | NA | NA | NA | NA |
| WCM0_LM1 | 19 | 16551617 | 17213341 | 0.2593 | 0.0434 | 1.2082 | 0.8529 | 1 | 1 |
| WCM0_LM1 | 19 | 17256224 | 18085909 | 0.9142 | 0.725 | 2.5448 | 0.7609 | 3 | 1 |
| WCM0_LM1 | 19 | 18092501 | 18538151 | 1.4431 | 1.2679 | 4.0126 | 0.8037 | 4 | 1 |
| WCM0_LM1 | 19 | 18538742 | 18870864 | 1.0318 | 0.8462 | 2.7673 | 0.8281 | 3 | 1 |
| WCM0_LM1 | 19 | 18876309 | 19011259 | 0.2326 | 0.0154 | NA | NA | NA | NA |
| WCM0_LM1 | 19 | 19014120 | 19310047 | 1.0372 | 0.8517 | 2.854 | 0.7553 | 3 | 1 |
| WCM0_LM1 | 19 | 19312537 | 19791033 | 1.4667 | 1.292 | 4.1096 | 0.7878 | 4 | 1 |
| WCM0_LM1 | 19 | 19822392 | 31048049 | 0.4849 | 0.2797 | 1.6939 | 0.7339 | 2 | 1 |
| WCM0_LM1 | 19 | 31767652 | 33140653 | −0.0464 | −0.2797 | 1.1559 | 0.4916 | 1 | 0 |
| WCM0_LM1 | 19 | 33149881 | 33202843 | 1.3048 | 1.1265 | NA | NA | NA | NA |
| WCM0_LM1 | 19 | 33321573 | 33700164 | −0.4408 | −0.7034 | 0.8175 | 0.4108 | 1 | 0 |
| WCM0_LM1 | 19 | 33701480 | 35263730 | 0.8236 | 0.6314 | NA | NA | NA | NA |
| WCM0_LM1 | 19 | 35420841 | 35660415 | −0.172 | −0.4137 | 0.9772 | 0.5241 | 1 | 1 |
| WCM0_LM1 | 19 | 35715714 | 35978637 | 0.7616 | 0.5672 | 2.0708 | 0.8925 | 2 | 1 |
| WCM0_LM1 | 19 | 35979514 | 36435775 | −0.4049 | −0.6645 | NA | NA | NA | NA |
| WCM0_LM1 | 19 | 36486327 | 36526526 | 0.6269 | 0.4275 | NA | NA | NA | NA |
| WCM0_LM1 | 19 | 36530332 | 36728082 | −0.5022 | −0.7702 | NA | NA | NA | NA |
| WCM0_LM1 | 19 | 36831321 | 36884965 | 0.4718 | 0.266 | NA | NA | NA | NA |
| WCM0_LM1 | 19 | 36896473 | 37006131 | −0.6818 | −0.9671 | NA | NA | NA | NA |
| WCM0_LM1 | 19 | 37037920 | 37441980 | 0.7207 | 0.5249 | NA | NA | NA | NA |
| WCM0_LM1 | 19 | 37580018 | 37721369 | 1.2136 | 1.0329 | NA | NA | NA | NA |
| WCM0_LM1 | 19 | 37726576 | 37880078 | 0.6072 | 0.407 | NA | NA | NA | NA |
| WCM0_LM1 | 19 | 37880434 | 37935813 | 1.2428 | 1.063 | NA | NA | NA | NA |
| WCM0_LM1 | 19 | 37961265 | 38746787 | 0.6828 | 0.4856 | 1.9667 | 0.8336 | 2 | 1 |
| WCM0_LM1 | 19 | 38755541 | 38849253 | −0.4168 | −0.6774 | NA | NA | NA | NA |
| WCM0_LM1 | 19 | 38850158 | 38886609 | 0.7684 | 0.5743 | NA | NA | NA | NA |
| WCM0_LM1 | 19 | 38893991 | 38912771 | −0.4964 | −0.7639 | NA | NA | NA | NA |
| WCM0_LM1 | 19 | 38916743 | 39138448 | 0.7391 | 0.5439 | 2.0742 | 0.8416 | 2 | 1 |
| WCM0_LM1 | 19 | 39191247 | 39522814 | −0.4277 | −0.6892 | NA | NA | NA | NA |
| WCM0_LM1 | 19 | 39575918 | 40331256 | 0.7486 | 0.5538 | 2.0761 | 0.8597 | 2 | 1 |
| WCM0_LM1 | 19 | 40331399 | 40408532 | 0.1184 | −0.105 | NA | NA | NA | NA |
| WCM0_LM1 | 19 | 40408821 | 40788489 | 0.7547 | 0.5601 | NA | NA | NA | NA |
| WCM0_LM1 | 19 | 40827864 | 40978572 | −0.4122 | −0.6724 | NA | NA | NA | NA |
| WCM0_LM1 | 19 | 40993722 | 41063232 | 0.7296 | 0.5341 | NA | NA | NA | NA |
| WCM0_LM1 | 19 | 41066224 | 41269485 | −0.4356 | −0.6978 | NA | NA | NA | NA |
| WCM0_LM1 | 19 | 41270961 | 41332543 | 0.6892 | 0.4922 | NA | NA | NA | NA |
| WCM0_LM1 | 19 | 41350673 | 41450693 | −0.4081 | −0.668 | NA | NA | NA | NA |
| WCM0_LM1 | 19 | 41497414 | 41699270 | 0.7777 | 0.5839 | NA | NA | NA | NA |
| WCM0_LM1 | 19 | 41700493 | 41869392 | −0.3887 | −0.647 | NA | NA | NA | NA |
| WCM0_LM1 | 19 | 41884253 | 42133278 | 0.8482 | 0.6569 | 2.1795 | 0.9738 | 2 | 1 |
| WCM0_LM1 | 19 | 42181442 | 42730593 | 0.3409 | 0.1291 | NA | NA | NA | NA |
| WCM0_LM1 | 19 | 42736814 | 42849016 | −4.1939 | NA | NA | NA | NA | NA |
| WCM0_LM1 | 19 | 42853828 | 43883187 | 0.3733 | 0.163 | 2.2415 | −0.0022 | 2 | 0 |
| WCM0_LM1 | 19 | 43910665 | 44174231 | −1.7227 | −2.1742 | NA | NA | NA | NA |
| WCM0_LM1 | 19 | 44222792 | 44377780 | 0.7646 | 0.5704 | NA | NA | NA | NA |
| WCM0_LM1 | 19 | 44377987 | 44733987 | 0.9044 | 0.7148 | 2.2748 | 1.0078 | 2 | 1 |
| WCM0_LM1 | 19 | 44739036 | 45349781 | 1.3197 | 1.1417 | 3.4264 | 0.9865 | 3 | 1 |
| WCM0_LM1 | 19 | 45368521 | 46113033 | 0.5813 | 0.3801 | 1.8078 | 0.795 | 2 | 1 |
| WCM0_LM1 | 19 | 46116948 | 46216642 | 1.2008 | 1.0198 | NA | NA | NA | NA |
| WCM0_LM1 | 19 | 46268920 | 46357733 | 0.6355 | 0.4364 | NA | NA | NA | NA |
| WCM0_LM1 | 19 | 46367703 | 46856963 | −0.2723 | −0.5213 | 0.9403 | 0.4532 | 1 | 0 |
| WCM0_LM1 | 19 | 46857286 | 47349292 | 0.6757 | 0.4782 | NA | NA | NA | NA |
| WCM0_LM1 | 19 | 47349444 | 47774912 | −0.4671 | −0.732 | NA | NA | NA | NA |
| WCM0_LM1 | 19 | 47813165 | 48902024 | 0.5967 | 0.3962 | 1.8142 | 0.8178 | 2 | 1 |
| WCM0_LM1 | 19 | 48908191 | 48925092 | −0.4592 | −0.7234 | NA | NA | NA | NA |
| WCM0_LM1 | 19 | 48945216 | 49016382 | 0.6515 | 0.4531 | NA | NA | NA | NA |
| WCM0_LM1 | 19 | 49055491 | 49611366 | −0.5004 | −0.7683 | 0.7816 | 0.3926 | 1 | 0 |
| WCM0_LM1 | 19 | 49611670 | 49839058 | 0.6628 | 0.4648 | NA | NA | NA | NA |
| WCM0_LM1 | 19 | 49840283 | 50885890 | −0.446 | −0.709 | 0.811 | 0.4124 | 1 | 0 |
| WCM0_LM1 | 19 | 50902197 | 51191346 | 0.7164 | 0.5204 | NA | NA | NA | NA |
| WCM0_LM1 | 19 | 51192076 | 51658570 | 1.3898 | 1.2134 | 3.6871 | 0.9505 | 4 | 1 |
| WCM0_LM1 | 19 | 51714527 | 52633748 | 0.8733 | 0.6827 | 2.2633 | 0.947 | 2 | 1 |
| WCM0_LM1 | 19 | 52658211 | 52723071 | −0.3422 | −0.5967 | NA | NA | NA | NA |
| WCM0_LM1 | 19 | 52723528 | 52919926 | 0.7005 | 0.5039 | NA | NA | NA | NA |
| WCM0_LM1 | 19 | 52934716 | 54567069 | −0.2902 | −0.5406 | 0.9177 | 0.4573 | 1 | 0 |
| WCM0_LM1 | 19 | 54610193 | 55147058 | 0.8328 | 0.6409 | 2.3137 | 0.8049 | 2 | 1 |
| WCM0_LM1 | 19 | 55174469 | 55341424 | 1.4071 | 1.2312 | NA | NA | NA | NA |
| WCM0_LM1 | 19 | 55351022 | 56206566 | −0.378 | −0.6354 | 0.8694 | 0.4181 | 1 | 0 |
| WCM0_LM1 | 19 | 56220245 | 56935565 | 0.7671 | 0.5729 | 2.0543 | 0.9207 | 2 | 1 |
| WCM0_LM1 | 19 | 56952671 | 57325073 | −0.1256 | −0.3642 | 1.0233 | 0.5305 | 1 | 1 |
| WCM0_LM1 | 19 | 57325834 | 58419984 | 0.4031 | 0.1942 | 1.3751 | 0.9131 | 1 | 1 |
| WCM0_LM1 | 19 | 58420360 | 59082661 | −0.0728 | −0.3079 | 1.1365 | 0.4791 | 1 | 0 |
| WCM0_LM1 | 2 | 41662 | 9002728 | 0.0358 | −0.1924 | 1.1507 | 0.5996 | 1 | 1 |
| WCM0_LM1 | 2 | 9004256 | 11597371 | −0.2145 | −0.4593 | 0.998 | 0.4567 | 1 | 0 |
| WCM0_LM1 | 2 | 11605925 | 21266363 | −0.0155 | −0.2469 | 1.1382 | 0.5473 | 1 | 1 |
| WCM0_LM1 | 2 | 23974884 | 29240779 | −0.277 | −0.5265 | 0.9318 | 0.4567 | 1 | 0 |
| WCM0_LM1 | 2 | 29245196 | 31805957 | 0.0172 | −0.2121 | 1.1197 | 0.6068 | 1 | 1 |
| WCM0_LM1 | 2 | 32093568 | 32891688 | −0.5018 | −0.7697 | NA | NA | NA | NA |
| WCM0_LM1 | 2 | 32897443 | 60983586 | −0.1864 | −0.4292 | 1.0131 | 0.4723 | 1 | 0 |
| WCM0_LM1 | 2 | 60987241 | 62112140 | −0.379 | −0.6364 | NA | NA | NA | NA |
| WCM0_LM1 | 2 | 62115667 | 65659098 | −0.1299 | −0.3688 | 1.0408 | 0.5081 | 1 | 1 |
| WCM0_LM1 | 2 | 66662948 | 69784074 | 0.328 | 0.1156 | 1.0902 | 1.0766 | 1 | 1 |
| WCM0_LM1 | 2 | 69870118 | 70529146 | −0.8451 | −1.1483 | NA | NA | NA | NA |
| WCM0_LM1 | 2 | 70692791 | 85051307 | −0.59 | −0.8662 | 1.0897 | 0.0075 | 1 | 0 |
| WCM0_LM1 | 2 | 85059231 | 90260116 | −0.7609 | −1.0546 | 0.9717 | −0.0088 | 1 | 0 |
| WCM0_LM1 | 2 | 90414130 | 91955353 | −0.385 | −0.6429 | NA | NA | NA | NA |
| WCM0_LM1 | 2 | 95537386 | 101612064 | 0.2475 | 0.031 | 1.0282 | 1.0153 | 1 | 1 |
| WCM0_LM1 | 2 | 101619137 | 112834865 | −0.0698 | −0.3047 | 1.0838 | 0.5354 | 1 | 1 |
| WCM0_LM1 | 2 | 112838625 | 116101463 | −0.5508 | −0.8233 | 1.1249 | 0.0054 | 1 | 0 |
| WCM0_LM1 | 2 | 116257180 | 120690162 | 0.1598 | −0.0614 | 0.9776 | 0.9392 | 1 | 1 |
| WCM0_LM1 | 2 | 120692501 | 149501221 | −0.6715 | −0.9557 | 1.0372 | −0.006 | 1 | 0 |
| WCM0_LM1 | 2 | 149511594 | 160836384 | 0.305 | 0.0914 | 1.0688 | 1.0621 | 1 | 1 |
| WCM0_LM1 | 2 | 160840487 | 170131630 | 0.441 | 0.2338 | 1.1808 | 1.171 | 1 | 1 |
| WCM0_LM1 | 2 | 170134365 | 176860300 | 0.2328 | 0.0156 | 1.0225 | 0.9991 | 1 | 1 |
| WCM0_LM1 | 2 | 176944789 | 183387074 | 0.5296 | 0.3263 | 1.259 | 1.2486 | 1 | 1 |
| WCM0_LM1 | 2 | 183582884 | 201677927 | 0.3293 | 0.1169 | 1.0844 | 1.0844 | 1 | 1 |
| WCM0_LM1 | 2 | 201680121 | 204360039 | 0.0769 | −0.1489 | 0.9105 | 0.8933 | 1 | 1 |
| WCM0_LM1 | 2 | 204571465 | 219082192 | 0.3192 | 0.1064 | 1.0822 | 1.0709 | 1 | 1 |
| WCM0_LM1 | 2 | 219090701 | 219689292 | 0.0778 | −0.148 | 0.9101 | 0.8949 | 1 | 1 |
| WCM0_LM1 | 2 | 219691684 | 223918019 | 0.2703 | 0.055 | 1.0454 | 1.0323 | 1 | 1 |
| WCM0_LM1 | 2 | 224462182 | 230787220 | −0.151 | −0.3913 | 1.0371 | 0.4878 | 1 | 0 |
| WCM0_LM1 | 2 | 230840964 | 234344499 | −0.7432 | −1.035 | 0.98 | −0.004 | 1 | 0 |
| WCM0_LM1 | 2 | 234345138 | 234656640 | −0.0758 | −0.311 | 1.0628 | 0.5493 | 1 | 1 |
| WCM0_LM1 | 2 | 234669089 | 234835260 | 0.0824 | −0.1431 | NA | NA | NA | NA |
| WCM0_LM1 | 2 | 234839404 | 242178149 | 0.3442 | 0.1325 | 1.1031 | 1.0893 | 1 | 1 |
| WCM0_LM1 | 2 | 242178251 | 242815433 | 0.1405 | −0.0817 | 0.9689 | 0.921 | 1 | 1 |
| WCM0_LM1 | 20 | 68375 | 168591 | 0.0729 | −0.1532 | NA | NA | NA | NA |
| WCM0_LM1 | 20 | 170225 | 871038 | −0.2172 | −0.4621 | 0.9782 | 0.4737 | 1 | 0 |
| WCM0_LM1 | 20 | 896641 | 2312944 | 0.0923 | −0.1326 | 1.1978 | 0.6266 | 1 | 1 |
| WCM0_LM1 | 20 | 2315739 | 6011924 | −0.2125 | −0.4571 | 0.9667 | 0.4902 | 1 | 0 |
| WCM0_LM1 | 20 | 6012767 | 25271125 | 0.0304 | −0.1982 | 1.1702 | 0.5731 | 1 | 1 |
| WCM0_LM1 | 20 | 25273088 | 25657666 | −0.1229 | −0.3613 | NA | NA | NA | NA |
| WCM0_LM1 | 20 | 25666234 | 34317461 | −0.2838 | −0.5337 | 0.9101 | 0.4715 | 1 | 0 |
| WCM0_LM1 | 20 | 34320036 | 35731214 | −0.0717 | −0.3066 | NA | NA | NA | NA |
| WCM0_LM1 | 20 | 35736994 | 45717954 | 0.2515 | 0.0353 | 1.0344 | 1.015 | 1 | 1 |
| WCM0_LM1 | 20 | 45725823 | 50713685 | 0.0399 | −0.1881 | 0.8908 | 0.8647 | 1 | 1 |
| WCM0_LM1 | 20 | 50713876 | 62076120 | 0.3031 | 0.0895 | 1.0718 | 1.0561 | 1 | 1 |
| WCM0_LM1 | 20 | 62076743 | 62680578 | 0.0518 | −0.1755 | 0.8986 | 0.8723 | 1 | 1 |
| WCM0_LM1 | 20 | 62694745 | 62904845 | 0.3983 | 0.1892 | NA | NA | NA | NA |
| WCM0_LM1 | 21 | 10862618 | 32493031 | 0.3938 | 0.1845 | 1.1471 | 1.1257 | 1 | 1 |
| WCM0_LM1 | 21 | 32496847 | 36088769 | 0.1727 | −0.0478 | 0.996 | 0.9389 | 1 | 1 |
| WCM0_LM1 | 21 | 36164321 | 37518393 | −0.7305 | −1.0209 | NA | NA | NA | NA |
| WCM0_LM1 | 21 | 37518756 | 38516988 | 0.0896 | −0.1355 | 0.9354 | 0.8853 | 1 | 1 |
| WCM0_LM1 | 21 | 38519818 | 48084196 | 0.3381 | 0.1262 | 1.104 | 1.0788 | 1 | 1 |
| WCM0_LM1 | 22 | 16255826 | 31724813 | −0.7461 | −1.0382 | 0.9779 | −0.0041 | 1 | 0 |
| WCM0_LM1 | 22 | 31731671 | 32352743 | −1.0116 | −1.3355 | NA | NA | NA | NA |
| WCM0_LM1 | 22 | 32435542 | 37964801 | −0.7354 | −1.0263 | 0.9889 | −0.007 | 1 | 0 |
| WCM0_LM1 | 22 | 37966313 | 43465814 | −0.9201 | −1.2323 | 0.8642 | −0.0129 | 1 | 0 |
| WCM0_LM1 | 22 | 43471514 | 51216346 | −0.7478 | −1.0401 | 0.9713 | 0.0013 | 1 | 0 |
| WCM0_LM1 | 3 | 361508 | 9290619 | −0.5025 | −0.7705 | 1.1669 | 0.0055 | 1 | 0 |
| WCM0_LM1 | 3 | 9406820 | 10331825 | −0.8686 | −1.1746 | 0.895 | −0.009 | 1 | 0 |
| WCM0_LM1 | 3 | 10342986 | 31789625 | −0.5926 | −0.869 | 1.0937 | 0.0014 | 1 | 0 |
| WCM0_LM1 | 3 | 31871595 | 33704926 | −0.8281 | −1.1293 | NA | NA | NA | NA |
| WCM0_LM1 | 3 | 33725922 | 47058770 | −0.5697 | −0.844 | 1.112 | 0.0022 | 1 | 0 |
| WCM0_LM1 | 3 | 47059126 | 50369546 | −0.8735 | −1.18 | 0.8993 | −0.0166 | 1 | 0 |
| WCM0_LM1 | 3 | 50374568 | 53381376 | −0.683 | −0.9684 | 1.0342 | −0.012 | 1 | 0 |
| WCM0_LM1 | 3 | 53529174 | 56605295 | −0.4625 | −0.727 | 1.1992 | 0.0091 | 1 | 0 |
| WCM0_LM1 | 3 | 56627002 | 62110081 | −0.8075 | −1.1063 | 0.9418 | −0.0128 | 1 | 0 |
| WCM0_LM1 | 3 | 62118295 | 68788363 | −0.4322 | −0.6941 | 1.2372 | −0.001 | 1 | 0 |
| WCM0_LM1 | 3 | 68802029 | 73673315 | −0.6562 | −0.9389 | 1.0293 | 0.014 | 1 | 0 |
| WCM0_LM1 | 3 | 73673686 | 74570191 | 0.9047 | 0.7152 | NA | NA | NA | NA |
| WCM0_LM1 | 3 | 75386371 | 93761981 | 0.2481 | 0.0316 | 1.0415 | 1.0028 | 1 | 1 |
| WCM0_LM1 | 3 | 93768279 | 122121578 | −0.5861 | −0.8619 | 1.0928 | 0.0077 | 1 | 0 |
| WCM0_LM1 | 3 | 122123173 | 122255087 | 0.1987 | −0.0203 | NA | NA | NA | NA |
| WCM0_LM1 | 3 | 122255221 | 141235173 | 0.327 | 0.1145 | 1.0826 | 1.0826 | 1 | 1 |
| WCM0_LM1 | 3 | 141248605 | 142525070 | 0.1045 | −0.1197 | 0.9252 | 0.9155 | 1 | 1 |
| WCM0_LM1 | 3 | 142537271 | 169487151 | 0.3684 | 0.1579 | 1.1239 | 1.1075 | 1 | 1 |
| WCM0_LM1 | 3 | 169492249 | 170185011 | 0.0536 | −0.1736 | 0.896 | 0.8772 | 1 | 1 |
| WCM0_LM1 | 3 | 170185177 | 182563258 | 0.2667 | 0.0512 | 1.0546 | 1.0176 | 1 | 1 |
| WCM0_LM1 | 3 | 182566359 | 186793363 | 0.1198 | −0.1035 | 0.9367 | 0.9249 | 1 | 1 |
| WCM0_LM1 | 3 | 186793617 | 194081657 | 0.4134 | 0.205 | 1.1756 | 1.1298 | 1 | 1 |
| WCM0_LM1 | 3 | 194081782 | 197847281 | 0.0649 | −0.1616 | 0.9007 | 0.8874 | 1 | 1 |
| WCM0_LM1 | 4 | 53435 | 437770 | 0.0436 | −0.1841 | NA | NA | NA | NA |
| WCM0_LM1 | 4 | 438219 | 3225686 | −0.2187 | −0.4638 | 0.9545 | 0.4956 | 1 | 0 |
| WCM0_LM1 | 4 | 3227429 | 38995310 | −0.0642 | −0.2987 | 1.0692 | 0.5567 | 1 | 1 |
| WCM0_LM1 | 4 | 38995574 | 41270007 | −0.4707 | −0.7359 | 0.7924 | 0.4084 | 1 | 0 |
| WCM0_LM1 | 4 | 41362851 | 44631587 | −1.3791 | −1.7597 | 0.582 | 0.0086 | 1 | 0 |
| WCM0_LM1 | 4 | 44637960 | 62933928 | −0.1185 | −0.3565 | 1.022 | 0.5401 | 1 | 1 |
| WCM0_LM1 | 4 | 62935916 | 71522102 | 0.3653 | 0.1546 | 1.1201 | 1.1061 | 1 | 1 |
| WCM0_LM1 | 4 | 71522962 | 71895110 | 0.0663 | −0.1601 | NA | NA | NA | NA |
| WCM0_LM1 | 4 | 72102311 | 73615956 | −0.4998 | −0.7676 | NA | NA | NA | NA |
| WCM0_LM1 | 4 | 73924000 | 75971468 | 0.3942 | 0.1849 | NA | NA | NA | NA |
| WCM0_LM1 | 4 | 76407858 | 77357396 | 0.1413 | −0.0808 | 0.9615 | 0.9295 | 1 | 1 |
| WCM0_LM1 | 4 | 77476824 | 82378694 | 0.3945 | 0.1852 | 1.1429 | 1.131 | 1 | 1 |
| WCM0_LM1 | 4 | 82380520 | 84518094 | 0.066 | −0.1605 | 0.9222 | 0.8672 | 1 | 1 |
| WCM0_LM1 | 4 | 84518575 | 106158383 | 0.3088 | 0.0955 | 1.0819 | 1.0549 | 1 | 1 |
| WCM0_LM1 | 4 | 106158540 | 106474017 | −0.5903 | −0.8665 | NA | NA | NA | NA |
| WCM0_LM1 | 4 | 106510485 | 177073042 | 0.2991 | 0.0853 | 1.0709 | 1.0509 | 1 | 1 |
| WCM0_LM1 | 4 | 177077291 | 178911613 | −0.6376 | −0.9184 | NA | NA | NA | NA |
| WCM0_LM1 | 4 | 183090475 | 190947586 | 0.056 | −0.171 | 0.9211 | 0.8553 | 1 | 1 |
| WCM0_LM1 | 5 | 140526 | 192227 | 0.1483 | −0.0734 | NA | NA | NA | NA |
| WCM0_LM1 | 5 | 192539 | 16465682 | 0.021 | −0.2082 | 1.1494 | 0.5819 | 1 | 1 |
| WCM0_LM1 | 5 | 16474924 | 68390019 | −0.0943 | −0.3308 | 1.0681 | 0.5221 | 1 | 1 |
| WCM0_LM1 | 5 | 68396773 | 68805616 | −0.466 | −0.7308 | NA | NA | NA | NA |
| WCM0_LM1 | 5 | 70308462 | 81460177 | −0.1124 | −0.3501 | 1.0357 | 0.5334 | 1 | 1 |
| WCM0_LM1 | 5 | 81474368 | 87962746 | 0.0532 | −0.1741 | 1.184 | 0.5887 | 1 | 1 |
| WCM0_LM1 | 5 | 88018483 | 122950033 | 0.3675 | 0.1569 | 1.1219 | 1.1079 | 1 | 1 |
| WCM0_LM1 | 5 | 123966364 | 132160881 | −0.5864 | −0.8622 | 1.1076 | −0.0074 | 1 | 0 |
| WCM0_LM1 | 5 | 132161095 | 134223668 | −0.8333 | −1.1351 | NA | NA | NA | NA |
| WCM0_LM1 | 5 | 134223862 | 135583202 | −0.3956 | −0.6544 | 1.2572 | 0.0134 | 1 | 0 |
| WCM0_LM1 | 5 | 135583453 | 139006308 | 0.0137 | −0.2159 | 0.8647 | 0.8574 | 1 | 1 |
| WCM0_LM1 | 5 | 139060264 | 146657755 | 0.3278 | 0.1153 | 1.0855 | 1.0809 | 1 | 1 |
| WCM0_LM1 | 5 | 146658785 | 148682032 | 0.4773 | 0.2717 | 1.2186 | 1.1959 | 1 | 1 |
| WCM0_LM1 | 5 | 148685952 | 170610418 | 0.2841 | 0.0695 | 1.0526 | 1.046 | 1 | 1 |
| WCM0_LM1 | 5 | 170626763 | 180166787 | 0.1416 | −0.0805 | 0.9547 | 0.9367 | 1 | 1 |
| WCM0_LM1 | 5 | 180166887 | 180687725 | 0.2885 | 0.0741 | NA | NA | NA | NA |
| WCM0_LM1 | 6 | 292598 | 17283043 | 0.2985 | 0.0846 | 1.0682 | 1.0526 | 1 | 1 |
| WCM0_LM1 | 6 | 17284886 | 18258589 | 0.0401 | −0.1879 | NA | NA | NA | NA |
| WCM0_LM1 | 6 | 18264205 | 30314223 | 0.2997 | 0.0858 | 1.0702 | 1.0524 | 1 | 1 |
| WCM0_LM1 | 6 | 30314598 | 37438826 | 0.1439 | −0.0781 | 0.9543 | 0.9403 | 1 | 1 |
| WCM0_LM1 | 6 | 37439651 | 41739604 | 0.3611 | 0.1503 | 1.1167 | 1.1028 | 1 | 1 |
| WCM0_LM1 | 6 | 41740715 | 43648794 | 0.0587 | −0.1682 | 0.9022 | 0.8777 | 1 | 1 |
| WCM0_LM1 | 6 | 43655425 | 44344794 | 0.2738 | 0.0587 | 1.0601 | 1.0229 | 1 | 1 |
| WCM0_LM1 | 6 | 44355554 | 71012669 | 0.407 | 0.1983 | 1.1557 | 1.1389 | 1 | 1 |
| WCM0_LM1 | 6 | 71138060 | 86197149 | 0.2871 | 0.0726 | 1.0759 | 1.0274 | 1 | 1 |
| WCM0_LM1 | 6 | 86199244 | 97730193 | 0.1319 | −0.0908 | 1.0441 | 0.8339 | 1 | 1 |
| WCM0_LM1 | 6 | 99282878 | 118972442 | 0.3109 | 0.0976 | 1.0756 | 1.0644 | 1 | 1 |
| WCM0_LM1 | 6 | 119221953 | 121768884 | −0.0998 | −0.3366 | NA | NA | NA | NA |
| WCM0_LM1 | 6 | 122720909 | 125144312 | 0.3713 | 0.1609 | NA | NA | NA | NA |
| WCM0_LM1 | 6 | 125231491 | 149395235 | −0.0414 | −0.2744 | 1.0941 | 0.5594 | 1 | 1 |
| WCM0_LM1 | 6 | 149691121 | 151914421 | −0.3073 | −0.5591 | 0.9025 | 0.4549 | 1 | 0 |
| WCM0_LM1 | 6 | 151917646 | 166755975 | −0.0931 | −0.3295 | 1.0512 | 0.5404 | 1 | 1 |
| WCM0_LM1 | 6 | 166778939 | 170893364 | 0.3954 | 0.1861 | 1.1579 | 1.1175 | 1 | 1 |
| WCM0_LM1 | 7 | 193625 | 4876274 | 0.1425 | −0.0796 | 0.9543 | 0.9384 | 1 | 1 |
| WCM0_LM1 | 7 | 4899762 | 6859485 | −0.0828 | −0.3185 | 0.813 | 0.7908 | 1 | 1 |
| WCM0_LM1 | 7 | 7118626 | 27196110 | 0.361 | 0.1502 | 1.1271 | 1.0923 | 1 | 1 |
| WCM0_LM1 | 7 | 27203195 | 27689282 | −0.4766 | −0.7423 | NA | NA | NA | NA |
| WCM0_LM1 | 7 | 27702390 | 29160547 | 0.3915 | 0.1821 | NA | NA | NA | NA |
| WCM0_LM1 | 7 | 29394282 | 38398370 | −0.5081 | −0.7766 | 1.1711 | −0.0036 | 1 | 0 |
| WCM0_LM1 | 7 | 38402533 | 39446346 | 0.4171 | 0.2089 | NA | NA | NA | NA |
| WCM0_LM1 | 7 | 39472846 | 45614312 | 0.2275 | 0.01 | 1.0239 | 0.99 | 1 | 1 |
| WCM0_LM1 | 7 | 45614707 | 55588806 | 0.4368 | 0.2294 | 1.176 | 1.1687 | 1 | 1 |
| WCM0_LM1 | 7 | 55750416 | 77584134 | −0.0049 | −0.2357 | 0.9097 | 0.7889 | 1 | 1 |
| WCM0_LM1 | 7 | 77648671 | 91502985 | 0.4046 | 0.1957 | 1.1561 | 1.1345 | 1 | 1 |
| WCM0_LM1 | 7 | 91503279 | 92151435 | 0.1573 | −0.0639 | NA | NA | NA | NA |
| WCM0_LM1 | 7 | 92157802 | 97363550 | 0.423 | 0.2151 | 1.1692 | 1.1523 | 1 | 1 |
| WCM0_LM1 | 7 | 97364132 | 102553714 | −0.0445 | −0.2777 | 0.9434 | 0.7064 | 1 | 1 |
| WCM0_LM1 | 7 | 102566827 | 108138018 | 0.231 | 0.0137 | 1.0201 | 0.9989 | 1 | 1 |
| WCM0_LM1 | 7 | 108142930 | 127847936 | 0.3859 | 0.1763 | 1.143 | 1.1169 | 1 | 1 |
| WCM0_LM1 | 7 | 127892093 | 129950740 | 0.0944 | −0.1304 | 0.9319 | 0.8952 | 1 | 1 |
| WCM0_LM1 | 7 | 129951972 | 137612947 | 0.3068 | 0.0934 | 1.0803 | 1.0534 | 1 | 1 |
| WCM0_LM1 | 7 | 137686437 | 140624387 | 0.0705 | −0.1557 | 0.9015 | 0.8939 | 1 | 1 |
| WCM0_LM1 | 7 | 140706285 | 142649654 | 0.5406 | 0.3378 | 1.6943 | 0.8333 | 2 | 1 |
| WCM0_LM1 | 7 | 142650982 | 146829590 | 0.3421 | 0.1304 | 1.1175 | 1.0716 | 1 | 1 |
| WCM0_LM1 | 7 | 146997203 | 148950613 | 0.0287 | −0.2 | 0.8881 | 0.853 | 1 | 1 |
| WCM0_LM1 | 7 | 148950958 | 158727128 | 0.2749 | 0.0598 | 1.0511 | 1.0336 | 1 | 1 |
| WCM0_LM1 | 7 | 158734759 | 158937481 | 0.5733 | 0.3718 | NA | NA | NA | NA |
| WCM0_LM1 | 8 | 192966 | 2092855 | −3.7508 | −6.0981 | NA | NA | NA | NA |
| WCM0_LM1 | 8 | 2796244 | 36698474 | −0.2129 | −0.4575 | 1.4622 | −0.0058 | 1 | 0 |
| WCM0_LM1 | 8 | 36703313 | 38265720 | 0.286 | 0.0715 | NA | NA | NA | NA |
| WCM0_LM1 | 8 | 38271278 | 41792140 | 0.4917 | 0.2868 | 1.3854 | 1.0545 | 1 | 1 |
| WCM0_LM1 | 8 | 41792390 | 48962477 | 0.2676 | 0.0522 | NA | NA | NA | NA |
| WCM0_LM1 | 8 | 49637377 | 67342273 | 0.5629 | 0.361 | 1.3859 | 1.1827 | 1 | 1 |
| WCM0_LM1 | 8 | 67342432 | 68204341 | 0.2589 | 0.0431 | NA | NA | NA | NA |
| WCM0_LM1 | 8 | 68208787 | 95272706 | 0.5545 | 0.3523 | 1.3909 | 1.1622 | 1 | 1 |
| WCM0_LM1 | 8 | 95384432 | 105601109 | 0.4098 | 0.2012 | 1.3132 | 0.9862 | 1 | 1 |
| WCM0_LM1 | 8 | 106430837 | 120577219 | −0.1567 | −0.3974 | 1.5142 | 0.0042 | 2 | 0 |
| WCM0_LM1 | 8 | 120580405 | 131414198 | −0.336 | −0.59 | 1.3283 | 4.00E−04 | 1 | 0 |
| WCM0_LM1 | 8 | 131792801 | 144295844 | −0.1572 | −0.3979 | 1.5137 | 0.0042 | 2 | 0 |
| WCM0_LM1 | 8 | 144297004 | 146032606 | −0.4141 | −0.6744 | 1.2623 | −0.0092 | 1 | 0 |
| WCM0_LM1 | 8 | 146032923 | 146279368 | −0.17 | −0.4117 | NA | NA | NA | NA |
| WCM0_LM1 | 9 | 117666 | 3395441 | −1.4181 | −1.8058 | 0.5789 | −0.0069 | 1 | 0 |
| WCM0_LM1 | 9 | 3828313 | 19032907 | −0.0195 | −0.2512 | 1.1339 | 0.5465 | 1 | 1 |
| WCM0_LM1 | 9 | 19049638 | 19550190 | −0.7882 | −1.0849 | NA | NA | NA | NA |
| WCM0_LM1 | 9 | 19573446 | 32784838 | −0.4504 | −0.7139 | 1.2124 | 0.007 | 1 | 0 |
| WCM0_LM1 | 9 | 32973565 | 39456961 | −0.6113 | −0.8895 | 1.0824 | −0.0029 | 1 | 0 |
| WCM0_LM1 | 9 | 40293070 | 99417036 | −0.0913 | −0.3276 | 1.0815 | 0.5123 | 1 | 1 |
| WCM0_LM1 | 9 | 99521250 | 114090419 | 0.3308 | 0.1185 | 1.0935 | 1.0776 | 1 | 1 |
| WCM0_LM1 | 9 | 114090711 | 116267769 | 0.1428 | −0.0793 | 0.9615 | 0.9316 | 1 | 1 |
| WCM0_LM1 | 9 | 116268683 | 123230115 | 0.4589 | 0.2526 | 1.2075 | 1.1752 | 1 | 1 |
| WCM0_LM1 | 9 | 123232440 | 127302282 | 0.2903 | 0.076 | 1.0641 | 1.0441 | 1 | 1 |
| WCM0_LM1 | 9 | 127306068 | 130830661 | 0.1164 | −0.1071 | 0.9402 | 0.9167 | 1 | 1 |
| WCM0_LM1 | 9 | 130830895 | 133072162 | −0.0393 | −0.2722 | 0.8325 | 0.8237 | 1 | 1 |
| WCM0_LM1 | 9 | 133327655 | 135254526 | 0.1056 | −0.1186 | 0.9211 | 0.9211 | 1 | 1 |
| WCM0_LM1 | 9 | 135261999 | 139405204 | 0.2839 | 0.0693 | 1.0635 | 1.0349 | 1 | 1 |
| WCM0_LM1 | 9 | 139405684 | 140695312 | 0.1359 | −0.0865 | 0.9557 | 0.9279 | 1 | 1 |
| WCM0_LM1 | 9 | 140706066 | 141071438 | 0.4125 | 0.2041 | NA | NA | NA | NA |
| WCM0_LM1 | X | 2700159 | 17820046 | 0.305 | 0.0914 | NA | NA | NA | NA |
| WCM0_LM1 | X | 18183275 | 24745209 | 0.1243 | −0.0988 | NA | NA | NA | NA |
| WCM0_LM1 | X | 24745986 | 40534530 | 0.3688 | 0.1583 | NA | NA | NA | NA |
| WCM0_LM1 | X | 40539076 | 49595690 | 0.0625 | −0.1642 | NA | NA | NA | NA |
| WCM0_LM1 | X | 49597215 | 70288184 | 0.2935 | 0.0794 | NA | NA | NA | NA |
| WCM0_LM1 | X | 70316613 | 70836559 | −0.0036 | −0.2343 | NA | NA | NA | NA |
| WCM0_LM1 | X | 70836914 | 101091121 | 0.2548 | 0.0387 | NA | NA | NA | NA |
| WCM0_LM1 | X | 101091727 | 110924478 | 0.4425 | 0.2354 | NA | NA | NA | NA |
| WCM0_LM1 | X | 110925433 | 124097582 | 0.1373 | −0.0851 | NA | NA | NA | NA |
| WCM0_LM1 | X | 125298700 | 140926085 | 0.8143 | 0.6218 | NA | NA | NA | NA |
| WCM0_LM1 | X | 140953207 | 152226437 | 1.0529 | 0.8679 | NA | NA | NA | NA |
| WCM0_LM1 | X | 152721728 | 154509348 | 0.777 | 0.5832 | NA | NA | NA | NA |
| WCM0_LM2 | 1 | 809001 | 11907304 | −0.1504 | −0.4673 | 0.9651 | 0.4816 | 1 | 0 |
| WCM0_LM2 | 1 | 11907604 | 19609254 | −0.6974 | −1.0532 | 0.9688 | −0.005 | 1 | 0 |
| WCM0_LM2 | 1 | 19611241 | 29652177 | −0.1841 | −0.503 | 0.9478 | 0.4635 | 1 | 0 |
| WCM0_LM2 | 1 | 31186427 | 31898727 | 0.6138 | 0.3307 | NA | NA | NA | NA |
| WCM0_LM2 | 1 | 31899500 | 32279901 | 0.8115 | 0.5345 | NA | NA | NA | NA |
| WCM0_LM2 | 1 | 32280190 | 33613323 | 0.5582 | 0.2732 | 1.5348 | 0.8822 | 2 | 1 |
| WCM0_LM2 | 1 | 33623876 | 35370912 | 0.9322 | 0.6585 | NA | NA | NA | NA |
| WCM0_LM2 | 1 | 35452725 | 41215223 | 0.6044 | 0.321 | 1.5383 | 0.96 | 2 | 1 |
| WCM0_LM2 | 1 | 41218830 | 44778831 | 0.7638 | 0.4854 | 1.7358 | 1.064 | 2 | 1 |
| WCM0_LM2 | 1 | 44785423 | 46775911 | 0.6076 | 0.3243 | NA | NA | NA | NA |
| WCM0_LM2 | 1 | 46806558 | 50310441 | 0.7974 | 0.52 | 1.7571 | 1.1108 | 2 | 1 |
| WCM0_LM2 | 1 | 50569670 | 50886936 | 0.5996 | 0.316 | NA | NA | NA | NA |
| WCM0_LM2 | 1 | 50907098 | 53516356 | 0.2081 | −0.0906 | NA | NA | NA | NA |
| WCM0_LM2 | 1 | 53527948 | 62149124 | 0.493 | 0.2057 | 1.1999 | 1.1067 | 1 | 1 |
| WCM0_LM2 | 1 | 62160464 | 64011649 | 0.2268 | −0.0711 | 0.9946 | 0.9092 | 1 | 1 |
| WCM0_LM2 | 1 | 64017384 | 91182664 | 0.4455 | 0.1564 | 1.1249 | 1.1041 | 1 | 1 |
| WCM0_LM2 | 1 | 91382527 | 120612005 | −0.5836 | −0.9299 | 1.0518 | −0.002 | 1 | 0 |
| WCM0_LM2 | 1 | 120612265 | 151752497 | −0.2417 | −0.564 | 0.8667 | 0.4861 | 1 | 0 |
| WCM0_LM2 | 1 | 151752629 | 153516409 | 0.0213 | −0.2863 | 1.113 | 0.527 | 1 | 1 |
| WCM0_LM2 | 1 | 153516420 | 156813181 | −0.2486 | −0.5714 | 0.8874 | 0.4586 | 1 | 0 |
| WCM0_LM2 | 1 | 156814048 | 160793962 | −0.0016 | −0.3103 | 1.0405 | 0.5724 | 1 | 1 |
| WCM0_LM2 | 1 | 160797514 | 161565408 | −0.2371 | −0.5592 | NA | NA | NA | NA |
| WCM0_LM2 | 1 | 161569620 | 205814681 | −0.0361 | −0.3467 | 1.0042 | 0.5686 | 1 | 1 |
| WCM0_LM2 | 1 | 205817056 | 210329130 | 0.0813 | −0.2234 | 1.0533 | 0.6599 | 1 | 1 |
| WCM0_LM2 | 1 | 210334068 | 233193025 | −0.063 | −0.3751 | 0.9824 | 0.5598 | 1 | 1 |
| WCM0_LM2 | 1 | 233225848 | 233394410 | −0.5806 | −0.9267 | NA | NA | NA | NA |
| WCM0_LM2 | 1 | 233394765 | 249212579 | −0.1496 | −0.4665 | 0.9097 | 0.5377 | 1 | 1 |
| WCM0_LM2 | 10 | 225958 | 5690932 | −0.4939 | −0.8333 | 1.128 | −0.0055 | 1 | 0 |
| WCM0_LM2 | 10 | 5693162 | 44112746 | −0.6941 | −1.0496 | 0.9711 | −0.0049 | 1 | 0 |
| WCM0_LM2 | 10 | 44139461 | 64565615 | −0.5075 | −0.8479 | 1.1045 | 0.0067 | 1 | 0 |
| WCM0_LM2 | 10 | 64572919 | 70998887 | −0.8897 | −1.2634 | 0.8482 | −0.0151 | 1 | 0 |
| WCM0_LM2 | 10 | 71000439 | 101162362 | −0.6174 | −0.9664 | 1.0265 | −0.0029 | 1 | 0 |
| WCM0_LM2 | 10 | 101163194 | 105218359 | −0.7465 | −1.1066 | 0.9401 | −0.0113 | 1 | 0 |
| WCM0_LM2 | 10 | 105254210 | 120460855 | −0.542 | −0.885 | 1.0838 | −8.00E−04 | 1 | 0 |
| WCM0_LM2 | 10 | 120467023 | 123844974 | −0.7122 | −1.0692 | 0.9741 | −0.0209 | 1 | 0 |
| WCM0_LM2 | 10 | 123845139 | 135373606 | −0.5134 | −0.8543 | 1.1003 | 0.0059 | 1 | 0 |
| WCM0_LM2 | 11 | 193096 | 1861131 | −0.6894 | −1.0445 | 0.9687 | 0.001 | 1 | 0 |
| WCM0_LM2 | 11 | 1861799 | 4185599 | −0.5767 | −0.9225 | 1.057 | −0.0018 | 1 | 0 |
| WCM0_LM2 | 11 | 4388724 | 8118404 | −0.4196 | −0.7537 | 1.1834 | 0.0028 | 1 | 0 |
| WCM0_LM2 | 11 | 8118761 | 45991385 | −0.5332 | −0.8756 | 1.0906 | −6.00E−04 | 1 | 0 |
| WCM0_LM2 | 11 | 45992748 | 55322837 | −0.7428 | −1.1025 | 0.932 | −6.00E−04 | 1 | 0 |
| WCM0_LM2 | 11 | 55339702 | 60899554 | −0.5585 | −0.9029 | 1.0704 | −7.00E−04 | 1 | 0 |
| WCM0_LM2 | 11 | 60899832 | 69487993 | −0.6937 | −1.0491 | 0.9767 | −0.0102 | 1 | 0 |
| WCM0_LM2 | 11 | 69489926 | 106849520 | 0.36 | 0.0676 | 1.0491 | 1.0469 | 1 | 1 |
| WCM0_LM2 | 11 | 106856799 | 110134872 | 0.1743 | −0.1259 | 0.9375 | 0.8953 | 1 | 1 |
| WCM0_LM2 | 11 | 110135573 | 116824829 | 0.3637 | 0.0715 | 1.0912 | 1.0104 | 1 | 1 |
| WCM0_LM2 | 11 | 116827661 | 117150687 | −0.7129 | −1.07 | NA | NA | NA | NA |
| WCM0_LM2 | 11 | 117150893 | 118656800 | −0.0634 | −0.3754 | 1.009 | 0.5328 | 1 | 1 |
| WCM0_LM2 | 11 | 118754629 | 119020937 | −0.772 | −1.1343 | NA | NA | NA | NA |
| WCM0_LM2 | 11 | 119024934 | 134257453 | 0.3883 | 0.0971 | 1.0718 | 1.0674 | 1 | 1 |
| WCM0_LM2 | 12 | 247582 | 459826 | −0.5089 | −0.8494 | NA | NA | NA | NA |
| WCM0_LM2 | 12 | 461326 | 1740480 | −0.7931 | −1.1574 | 0.8883 | 0.0083 | 1 | 0 |
| WCM0_LM2 | 12 | 1741845 | 6155858 | −0.5146 | −0.8555 | 1.106 | −7.00E−04 | 1 | 0 |
| WCM0_LM2 | 12 | 6161693 | 9225264 | −0.738 | −1.0973 | 0.9408 | −0.006 | 1 | 0 |
| WCM0_LM2 | 12 | 9227195 | 9578319 | −0.5413 | −0.8843 | NA | NA | NA | NA |
| WCM0_LM2 | 12 | 9659718 | 9713413 | 0.3602 | 0.0679 | NA | NA | NA | NA |
| WCM0_LM2 | 12 | 9723417 | 18241826 | −0.5684 | −0.9135 | 1.0623 | −4.00E−04 | 1 | 0 |
| WCM0_LM2 | 12 | 18242148 | 20905250 | −1.3071 | −1.73 | NA | NA | NA | NA |
| WCM0_LM2 | 12 | 20905494 | 21069133 | −0.4659 | −0.8033 | NA | NA | NA | NA |
| WCM0_LM2 | 12 | 21168677 | 22837880 | −1.357 | −1.7869 | 0.5682 | 0.0114 | 1 | 0 |
| WCM0_LM2 | 12 | 23687201 | 24365376 | −0.2943 | −0.62 | NA | NA | NA | NA |
| WCM0_LM2 | 12 | 24736749 | 25348052 | −1.4386 | −1.8807 | NA | NA | NA | NA |
| WCM0_LM2 | 12 | 25356916 | 28605426 | −0.0099 | −0.3191 | 1.0394 | 0.5638 | 1 | 1 |
| WCM0_LM2 | 12 | 28637090 | 31435831 | 0.3937 | 0.1026 | 1.1239 | 1.0235 | 1 | 1 |
| WCM0_LM2 | 12 | 31440687 | 32908129 | 0.0869 | −0.2174 | 0.8953 | 0.8249 | 1 | 1 |
| WCM0_LM2 | 12 | 32908360 | 40761516 | 0.3641 | 0.0719 | NA | NA | NA | NA |
| WCM0_LM2 | 12 | 41302275 | 41337822 | −0.2409 | −0.5632 | NA | NA | NA | NA |
| WCM0_LM2 | 12 | 41352954 | 48743770 | −0.464 | −0.8012 | 1.1371 | 0.0107 | 1 | 0 |
| WCM0_LM2 | 12 | 48866585 | 51757916 | −0.6938 | −1.0493 | 0.9682 | −0.0018 | 1 | 0 |
| WCM0_LM2 | 12 | 51758127 | 53298685 | −0.4231 | −0.7574 | 1.1862 | −0.003 | 1 | 0 |
| WCM0_LM2 | 12 | 53298775 | 54107503 | −0.6992 | −1.0551 | 0.9801 | −0.0176 | 1 | 0 |
| WCM0_LM2 | 12 | 54107929 | 56078966 | −0.5129 | −0.8537 | NA | NA | NA | NA |
| WCM0_LM2 | 12 | 56079067 | 57181011 | −0.7208 | −1.0786 | 0.9758 | −0.0288 | 1 | 0 |
| WCM0_LM2 | 12 | 57317619 | 77240448 | −0.5494 | −0.8931 | 1.0838 | −0.0069 | 1 | 0 |
| WCM0_LM2 | 12 | 77242040 | 93157851 | −0.446 | −0.7819 | 1.1525 | 0.0107 | 1 | 0 |
| WCM0_LM2 | 12 | 93162860 | 110240989 | −0.5939 | −0.941 | 1.044 | −0.0023 | 1 | 0 |
| WCM0_LM2 | 12 | 110246195 | 112915456 | −0.8319 | −1.1999 | NA | NA | NA | NA |
| WCM0_LM2 | 12 | 112915755 | 120582744 | −0.6181 | −0.9672 | 1.0259 | −0.0029 | 1 | 0 |
| WCM0_LM2 | 12 | 120583045 | 124303507 | −0.7965 | −1.1612 | 0.9105 | −0.0162 | 1 | 0 |
| WCM0_LM2 | 12 | 124303750 | 133779296 | −0.5908 | −0.9376 | 1.0464 | −0.0022 | 1 | 0 |
| WCM0_LM2 | 13 | 19705463 | 61989209 | −0.5704 | −0.9157 | 1.0618 | −0.0016 | 1 | 0 |
| WCM0_LM2 | 13 | 63745874 | 70681840 | −1.2897 | −1.7102 | NA | NA | NA | NA |
| WCM0_LM2 | 13 | 72014840 | 73637958 | −0.4773 | −0.8155 | NA | NA | NA | NA |
| WCM0_LM2 | 13 | 73649842 | 80911767 | −0.3128 | −0.6396 | 1.2762 | 0.0075 | 1 | 0 |
| WCM0_LM2 | 13 | 84453597 | 115089411 | 0.5121 | 0.2254 | 2.3436 | −0.0053 | 2 | 0 |
| WCM0_LM2 | 14 | 19553736 | 20692291 | 0.2546 | −0.0421 | 0.9946 | 0.948 | 1 | 1 |
| WCM0_LM2 | 14 | 20692574 | 22180535 | 0.1361 | −0.1659 | 0.8988 | 0.8839 | 1 | 1 |
| WCM0_LM2 | 14 | 22180788 | 23057900 | 0.3578 | 0.0654 | 1.0681 | 1.0246 | 1 | 1 |
| WCM0_LM2 | 14 | 23067224 | 23745364 | 0.0894 | −0.2148 | 0.8725 | 0.8508 | 1 | 1 |
| WCM0_LM2 | 14 | 23745512 | 31188516 | 0.2959 | 0.001 | 1.0142 | 0.9871 | 1 | 1 |
| WCM0_LM2 | 14 | 31191060 | 51190262 | 0.0844 | −0.2201 | 0.8657 | 0.8513 | 1 | 1 |
| WCM0_LM2 | 14 | 51190398 | 73238595 | 0.2306 | −0.0671 | 0.9605 | 0.9486 | 1 | 1 |
| WCM0_LM2 | 14 | 73360818 | 75747783 | 0.0306 | −0.2765 | 0.8256 | 0.8256 | 1 | 1 |
| WCM0_LM2 | 14 | 75748098 | 95267286 | 0.1876 | −0.1121 | 0.931 | 0.9194 | 1 | 1 |
| WCM0_LM2 | 14 | 95556866 | 95599710 | −0.6663 | −1.0194 | NA | NA | NA | NA |
| WCM0_LM2 | 14 | 95658021 | 102028638 | 0.3373 | 0.044 | 1.0621 | 0.9999 | 1 | 1 |
| WCM0_LM2 | 14 | 102228256 | 107283105 | 0.1716 | −0.1288 | 0.9624 | 0.8668 | 1 | 1 |
| WCM0_LM2 | 15 | 20169960 | 40764530 | 0.3199 | 0.0259 | 1.104 | 0.9323 | 1 | 1 |
| WCM0_LM2 | 15 | 40846235 | 42058949 | 0.0588 | −0.2469 | NA | NA | NA | NA |
| WCM0_LM2 | 15 | 42059002 | 42491761 | 0.3235 | 0.0297 | 1.0399 | 1.0017 | 1 | 1 |
| WCM0_LM2 | 15 | 42492119 | 45142593 | 0.1543 | −0.1468 | NA | NA | NA | NA |
| WCM0_LM2 | 15 | 45206957 | 50339654 | 0.3901 | 0.099 | 1.0955 | 1.0465 | 1 | 1 |
| WCM0_LM2 | 15 | 50366340 | 59974617 | 0.2178 | −0.0806 | 0.9457 | 0.9457 | 1 | 1 |
| WCM0_LM2 | 15 | 60297219 | 63967333 | 0.3632 | 0.071 | 1.1018 | 0.9991 | 1 | 1 |
| WCM0_LM2 | 15 | 63970196 | 66853583 | 0.1355 | −0.1665 | NA | NA | NA | NA |
| WCM0_LM2 | 15 | 66855944 | 102359217 | 0.2825 | −0.013 | 0.9941 | 0.9879 | 1 | 1 |
| WCM0_LM2 | 16 | 79642 | 3274032 | −0.6544 | −1.0065 | 1.0048 | −0.0093 | 1 | 0 |
| WCM0_LM2 | 16 | 3274264 | 47252861 | −0.7855 | −1.1491 | 0.9139 | −0.0121 | 1 | 0 |
| WCM0_LM2 | 16 | 47271957 | 67360773 | −0.6283 | −0.9782 | 1.0162 | −0.001 | 1 | 0 |
| WCM0_LM2 | 16 | 67375880 | 70594429 | −0.7866 | −1.1503 | 0.9127 | −0.0116 | 1 | 0 |
| WCM0_LM2 | 16 | 70595639 | 90162210 | −0.6527 | −1.0046 | 1.0007 | −0.0039 | 1 | 0 |
| WCM0_LM2 | 17 | 131595 | 7579831 | 0.1595 | −0.1415 | 0.9104 | 0.9028 | 1 | 1 |
| WCM0_LM2 | 17 | 7592149 | 7951793 | −0.6695 | −1.0229 | NA | NA | NA | NA |
| WCM0_LM2 | 17 | 7976111 | 8647931 | −0.1607 | −0.4782 | 0.9731 | 0.4627 | 1 | 0 |
| WCM0_LM2 | 17 | 8700793 | 28029992 | 0.1877 | −0.112 | 0.9824 | 0.8683 | 1 | 1 |
| WCM0_LM2 | 17 | 28120878 | 28773021 | 0.7147 | 0.4348 | NA | NA | NA | NA |
| WCM0_LM2 | 17 | 28776656 | 47501985 | −0.7385 | −1.0978 | 0.9456 | −0.0111 | 1 | 0 |
| WCM0_LM2 | 17 | 47572774 | 56647766 | −0.6128 | −0.9615 | 1.0299 | −0.0028 | 1 | 0 |
| WCM0_LM2 | 17 | 56649426 | 59857643 | −0.7997 | −1.1646 | 0.9031 | −0.0109 | 1 | 0 |
| WCM0_LM2 | 17 | 59858254 | 72954993 | 0.211 | −0.0876 | 0.9529 | 0.9293 | 1 | 1 |
| WCM0_LM2 | 17 | 72955118 | 76968818 | 0.128 | −0.1744 | 0.8917 | 0.8806 | 1 | 1 |
| WCM0_LM2 | 17 | 76969185 | 81052333 | 0.2439 | −0.0532 | 0.9678 | 0.9597 | 1 | 1 |
| WCM0_LM2 | 18 | 47641 | 14613577 | 0.8024 | 0.5251 | 1.9069 | 0.9711 | 2 | 1 |
| WCM0_LM2 | 18 | 18539788 | 22672060 | −0.7723 | −1.1347 | 0.9366 | −0.0258 | 1 | 0 |
| WCM0_LM2 | 18 | 22775185 | 74980776 | −0.543 | −0.8862 | 1.083 | −9.00E−04 | 1 | 0 |
| WCM0_LM2 | 18 | 74980877 | 77287496 | 0.8278 | 0.5512 | NA | NA | NA | NA |
| WCM0_LM2 | 18 | 77439995 | 78005214 | −0.5101 | −0.8507 | NA | NA | NA | NA |
| WCM0_LM2 | 19 | 281360 | 327207 | 0.3508 | 0.0581 | NA | NA | NA | NA |
| WCM0_LM2 | 19 | 327997 | 6374260 | 0.1212 | −0.1815 | 0.8892 | 0.8744 | 1 | 1 |
| WCM0_LM2 | 19 | 6374445 | 7809842 | 0.3837 | 0.0923 | 1.3042 | 0.8279 | 1 | 1 |
| WCM0_LM2 | 19 | 7810668 | 8203328 | 0.9435 | 0.6701 | 2.357 | 0.8254 | 2 | 1 |
| WCM0_LM2 | 19 | 8206559 | 8326982 | 0.8088 | 0.5317 | NA | NA | NA | NA |
| WCM0_LM2 | 19 | 8367282 | 8495625 | 0.2638 | −0.0326 | 1.1612 | 0.7942 | 1 | 1 |
| WCM0_LM2 | 19 | 8503458 | 9771427 | 0.8897 | 0.6149 | 2.2409 | 0.822 | 2 | 1 |
| WCM0_LM2 | 19 | 9801434 | 10504058 | 0.338 | 0.0448 | 1.1957 | 0.8673 | 1 | 1 |
| WCM0_LM2 | 19 | 10505720 | 10692406 | 0.8764 | 0.6012 | NA | NA | NA | NA |
| WCM0_LM2 | 19 | 10694364 | 11326479 | 1.2658 | 1 | 3.2474 | 0.7525 | 3 | 1 |
| WCM0_LM2 | 19 | 11327571 | 11348379 | 0.9272 | 0.6533 | NA | NA | NA | NA |
| WCM0_LM2 | 19 | 11348911 | 11598567 | 0.3782 | 0.0865 | NA | NA | NA | NA |
| WCM0_LM2 | 19 | 11606822 | 12089946 | 1.2131 | 0.9461 | NA | NA | NA | NA |
| WCM0_LM2 | 19 | 12125788 | 12637726 | 0.2532 | −0.0436 | NA | NA | NA | NA |
| WCM0_LM2 | 19 | 12637834 | 12886188 | 0.9148 | 0.6406 | NA | NA | NA | NA |
| WCM0_LM2 | 19 | 12902697 | 14703188 | 0.4454 | 0.1563 | 1.2474 | 0.9815 | 1 | 1 |
| WCM0_LM2 | 19 | 14705373 | 15221187 | 0.8715 | 0.5961 | 2.2231 | 0.8002 | 2 | 1 |
| WCM0_LM2 | 19 | 15221544 | 15853070 | 0.4415 | 0.1523 | 1.342 | 0.8807 | 1 | 1 |
| WCM0_LM2 | 19 | 15882836 | 16222893 | 0.9507 | 0.6774 | 2.3673 | 0.8313 | 2 | 1 |
| WCM0_LM2 | 19 | 16229008 | 16436714 | 0.3393 | 0.0461 | NA | NA | NA | NA |
| WCM0_LM2 | 19 | 16437661 | 16551608 | 0.9119 | 0.6376 | NA | NA | NA | NA |
| WCM0_LM2 | 19 | 16552721 | 17213366 | 0.3713 | 0.0794 | 1.2411 | 0.8721 | 1 | 1 |
| WCM0_LM2 | 19 | 17256273 | 18085936 | 0.8722 | 0.5969 | 2.2352 | 0.7898 | 2 | 1 |
| WCM0_LM2 | 19 | 18092703 | 18530494 | 1.3071 | 1.0421 | 3.263 | 0.8554 | 3 | 1 |
| WCM0_LM2 | 19 | 18538157 | 18870989 | 1.014 | 0.7424 | 2.4445 | 0.9014 | 2 | 1 |
| WCM0_LM2 | 19 | 18876309 | 19011238 | 0.4846 | 0.197 | NA | NA | NA | NA |
| WCM0_LM2 | 19 | 19014125 | 19310047 | 0.9683 | 0.6956 | 2.382 | 0.857 | 2 | 1 |
| WCM0_LM2 | 19 | 19312537 | 19681630 | 1.3499 | 1.0857 | 3.3842 | 0.8608 | 3 | 1 |
| WCM0_LM2 | 19 | 19710129 | 20003109 | 0.9761 | 0.7036 | NA | NA | NA | NA |
| WCM0_LM2 | 19 | 20003272 | 33200249 | 0.3451 | 0.0522 | 1.3938 | 0.6798 | 1 | 1 |
| WCM0_LM2 | 19 | 33202813 | 33701353 | −0.3655 | −0.6958 | 0.8296 | 0.4052 | 1 | 0 |
| WCM0_LM2 | 19 | 33701812 | 35263693 | 0.6075 | 0.3241 | NA | NA | NA | NA |
| WCM0_LM2 | 19 | 35420841 | 35660525 | −0.0949 | −0.4086 | 0.9831 | 0.5236 | 1 | 1 |
| WCM0_LM2 | 19 | 35715740 | 35978567 | 0.5806 | 0.2963 | 1.5315 | 0.9246 | 2 | 1 |
| WCM0_LM2 | 19 | 35979523 | 36435937 | −0.2359 | −0.5579 | NA | NA | NA | NA |
| WCM0_LM2 | 19 | 36486436 | 36530798 | 0.5509 | 0.2656 | NA | NA | NA | NA |
| WCM0_LM2 | 19 | 36545051 | 36728204 | −0.3637 | −0.694 | NA | NA | NA | NA |
| WCM0_LM2 | 19 | 36831136 | 36896567 | 0.256 | −0.0406 | NA | NA | NA | NA |
| WCM0_LM2 | 19 | 36898864 | 37006131 | −0.6925 | −1.0479 | NA | NA | NA | NA |
| WCM0_LM2 | 19 | 37037960 | 38746894 | 0.4726 | 0.1846 | 1.4746 | 0.7984 | 1 | 1 |
| WCM0_LM2 | 19 | 38755533 | 38845439 | −0.3094 | −0.636 | NA | NA | NA | NA |
| WCM0_LM2 | 19 | 38847156 | 38886271 | 0.5556 | 0.2704 | NA | NA | NA | NA |
| WCM0_LM2 | 19 | 38886526 | 38912586 | −0.1959 | −0.5155 | NA | NA | NA | NA |
| WCM0_LM2 | 19 | 38916756 | 39138447 | 0.5188 | 0.2324 | 1.4217 | 0.9278 | 1 | 1 |
| WCM0_LM2 | 19 | 39191210 | 39522789 | −0.3243 | −0.6519 | NA | NA | NA | NA |
| WCM0_LM2 | 19 | 39575916 | 40762969 | 0.5604 | 0.2755 | 1.5648 | 0.8561 | 2 | 1 |
| WCM0_LM2 | 19 | 40771119 | 40978551 | −0.2495 | −0.5724 | NA | NA | NA | NA |
| WCM0_LM2 | 19 | 40993675 | 41063170 | 0.5773 | 0.293 | NA | NA | NA | NA |
| WCM0_LM2 | 19 | 41066221 | 41271087 | −0.2348 | −0.5567 | NA | NA | NA | NA |
| WCM0_LM2 | 19 | 41281554 | 41332543 | 0.6147 | 0.3316 | NA | NA | NA | NA |
| WCM0_LM2 | 19 | 41350686 | 41450715 | −0.207 | −0.5272 | NA | NA | NA | NA |
| WCM0_LM2 | 19 | 41497353 | 41633884 | 0.519 | 0.2326 | NA | NA | NA | NA |
| WCM0_LM2 | 19 | 41699292 | 41863777 | −0.2368 | −0.5589 | NA | NA | NA | NA |
| WCM0_LM2 | 19 | 41869392 | 42133329 | 0.6507 | 0.3688 | 1.5579 | 1.0247 | 2 | 1 |
| WCM0_LM2 | 19 | 42181360 | 42392792 | −0.0622 | −0.3742 | NA | NA | NA | NA |
| WCM0_LM2 | 19 | 42396179 | 42598073 | 0.6228 | 0.34 | NA | NA | NA | NA |
| WCM0_LM2 | 19 | 42599640 | 42730600 | 0.0519 | −0.2542 | NA | NA | NA | NA |
| WCM0_LM2 | 19 | 42736267 | 42848993 | −4.3138 | −9.9291 | NA | NA | NA | NA |
| WCM0_LM2 | 19 | 42853725 | 43032546 | 0.0238 | −0.2837 | NA | NA | NA | NA |
| WCM0_LM2 | 19 | 43087484 | 43236931 | −1.4938 | −1.9445 | NA | NA | NA | NA |
| WCM0_LM2 | 19 | 43237172 | 43910649 | 0.0562 | −0.2496 | 1.7062 | −0.024 | 2 | 0 |
| WCM0_LM2 | 19 | 43920036 | 44174085 | −1.6371 | −2.1122 | NA | NA | NA | NA |
| WCM0_LM2 | 19 | 44222890 | 44733891 | 0.6261 | 0.3434 | 1.6002 | 0.9372 | 2 | 1 |
| WCM0_LM2 | 19 | 44739091 | 45349799 | 0.9709 | 0.6982 | 2.2914 | 0.9535 | 2 | 1 |
| WCM0_LM2 | 19 | 45368497 | 46112999 | 0.4795 | 0.1917 | 1.341 | 0.9432 | 1 | 1 |
| WCM0_LM2 | 19 | 46116816 | 46216642 | 1.0285 | 0.7573 | NA | NA | NA | NA |
| WCM0_LM2 | 19 | 46268872 | 46367703 | 0.5708 | 0.2862 | NA | NA | NA | NA |
| WCM0_LM2 | 19 | 46375421 | 46879824 | −0.2194 | −0.5404 | 0.9188 | 0.4564 | 1 | 0 |
| WCM0_LM2 | 19 | 46886612 | 47353975 | 0.5132 | 0.2266 | NA | NA | NA | NA |
| WCM0_LM2 | 19 | 47421971 | 47774907 | −0.2455 | −0.5681 | NA | NA | NA | NA |
| WCM0_LM2 | 19 | 47813165 | 48949595 | 0.4546 | 0.1659 | 1.4267 | 0.817 | 1 | 1 |
| WCM0_LM2 | 19 | 48949766 | 49016442 | 0.5572 | 0.2722 | NA | NA | NA | NA |
| WCM0_LM2 | 19 | 49055587 | 49611643 | −0.2577 | −0.5811 | 0.8716 | 0.4654 | 1 | 0 |
| WCM0_LM2 | 19 | 49618175 | 49813726 | 0.5037 | 0.2168 | NA | NA | NA | NA |
| WCM0_LM2 | 19 | 49814384 | 50885847 | −0.2731 | −0.5974 | 0.8802 | 0.4417 | 1 | 0 |
| WCM0_LM2 | 19 | 50902264 | 51191193 | 0.5377 | 0.252 | NA | NA | NA | NA |
| WCM0_LM2 | 19 | 51192067 | 51658582 | 1.0986 | 0.829 | 2.6982 | 0.8547 | 3 | 1 |
| WCM0_LM2 | 19 | 51714527 | 52550080 | 0.6571 | 0.3753 | 1.6747 | 0.9196 | 2 | 1 |
| WCM0_LM2 | 19 | 52618044 | 52633797 | 0.4371 | 0.1478 | NA | NA | NA | NA |
| WCM0_LM2 | 19 | 52658405 | 52723015 | −0.1815 | −0.5003 | NA | NA | NA | NA |
| WCM0_LM2 | 19 | 52723436 | 52937271 | 0.4464 | 0.1574 | NA | NA | NA | NA |
| WCM0_LM2 | 19 | 52938295 | 54567035 | −0.2621 | −0.5858 | 0.8617 | 0.4709 | 1 | 0 |
| WCM0_LM2 | 19 | 54610127 | 55179441 | 0.6446 | 0.3625 | 1.7143 | 0.8569 | 2 | 1 |
| WCM0_LM2 | 19 | 55286669 | 56206477 | −0.2632 | −0.5869 | 0.8822 | 0.4493 | 1 | 0 |
| WCM0_LM2 | 19 | 56220245 | 56935545 | 0.5039 | 0.217 | 1.4849 | 0.8396 | 1 | 1 |
| WCM0_LM2 | 19 | 56952645 | 57325655 | −0.0895 | −0.403 | 0.9646 | 0.548 | 1 | 1 |
| WCM0_LM2 | 19 | 57326077 | 58439306 | 0.4424 | 0.1533 | 1.2863 | 0.9379 | 1 | 1 |
| WCM0_LM2 | 19 | 58441804 | 59082661 | −0.0393 | −0.3501 | 1.0712 | 0.4979 | 1 | 0 |
| WCM0_LM2 | 2 | 41647 | 21266460 | −0.0169 | −0.3265 | 1.056 | 0.539 | 1 | 1 |
| WCM0_LM2 | 2 | 23974938 | 29432678 | −0.1904 | −0.5097 | 0.9122 | 0.4926 | 1 | 0 |
| WCM0_LM2 | 2 | 29436953 | 31625967 | 0.0138 | −0.2942 | 1.071 | 0.5601 | 1 | 1 |
| WCM0_LM2 | 2 | 31628860 | 44589177 | −0.2338 | −0.5556 | 0.8955 | 0.4652 | 1 | 0 |
| WCM0_LM2 | 2 | 44599846 | 55277374 | −0.0386 | −0.3493 | 1.0329 | 0.537 | 1 | 1 |
| WCM0_LM2 | 2 | 55402980 | 63815423 | −0.2346 | −0.5565 | 0.9116 | 0.4483 | 1 | 0 |
| WCM0_LM2 | 2 | 63816121 | 68274489 | −0.0746 | −0.3872 | 0.9895 | 0.5396 | 1 | 1 |
| WCM0_LM2 | 2 | 68352450 | 69784064 | 0.4272 | 0.1375 | 1.1217 | 1.0783 | 1 | 1 |
| WCM0_LM2 | 2 | 69870168 | 91766588 | −0.6733 | −1.0269 | 0.9859 | −0.0044 | 1 | 0 |
| WCM0_LM2 | 2 | 91777703 | 101619136 | 0.2574 | −0.0392 | 0.9864 | 0.96 | 1 | 1 |
| WCM0_LM2 | 2 | 101620767 | 112942904 | −0.125 | −0.4404 | 0.9931 | 0.4808 | 1 | 0 |
| WCM0_LM2 | 2 | 112944630 | 116257108 | −0.6158 | −0.9647 | 1.0249 | −1.00E−04 | 1 | 0 |
| WCM0_LM2 | 2 | 116283478 | 118578918 | −0.1449 | −0.4615 | NA | NA | NA | NA |
| WCM0_LM2 | 2 | 118579252 | 120690075 | 0.243 | −0.0542 | 0.9872 | 0.939 | 1 | 1 |
| WCM0_LM2 | 2 | 120692535 | 149501148 | −0.7183 | −1.0759 | 0.9543 | −0.0055 | 1 | 0 |
| WCM0_LM2 | 2 | 149511523 | 160811874 | 0.3254 | 0.0317 | 1.0296 | 1.0147 | 1 | 1 |
| WCM0_LM2 | 2 | 160812225 | 170145592 | 0.4488 | 0.1599 | 1.1322 | 1.1021 | 1 | 1 |
| WCM0_LM2 | 2 | 170147342 | 179391714 | 0.2926 | −0.0025 | 1.0087 | 0.9879 | 1 | 1 |
| WCM0_LM2 | 2 | 179392019 | 182543506 | 0.5387 | 0.253 | 1.2176 | 1.1658 | 1 | 1 |
| WCM0_LM2 | 2 | 182756817 | 201680462 | 0.3218 | 0.0279 | 1.028 | 1.011 | 1 | 1 |
| WCM0_LM2 | 2 | 201681077 | 204599484 | 0.1057 | −0.1977 | 0.8756 | 0.8683 | 1 | 1 |
| WCM0_LM2 | 2 | 204732693 | 219029690 | 0.3459 | 0.053 | 1.0449 | 1.0298 | 1 | 1 |
| WCM0_LM2 | 2 | 219029752 | 219692133 | 0.1526 | −0.1486 | 0.9087 | 0.8955 | 1 | 1 |
| WCM0_LM2 | 2 | 219692363 | 223918019 | 0.3296 | 0.036 | 1.0423 | 1.0082 | 1 | 1 |
| WCM0_LM2 | 2 | 224462246 | 230744708 | −0.1511 | −0.4681 | 0.9232 | 0.5226 | 1 | 1 |
| WCM0_LM2 | 2 | 230787332 | 234343433 | −0.754 | −1.1148 | 0.9274 | −0.0039 | 1 | 0 |
| WCM0_LM2 | 2 | 234344510 | 234656640 | −0.0639 | −0.376 | 1.0232 | 0.518 | 1 | 1 |
| WCM0_LM2 | 2 | 234669142 | 234858807 | 0.0459 | −0.2605 | NA | NA | NA | NA |
| WCM0_LM2 | 2 | 234862657 | 242021201 | 0.3767 | 0.085 | 1.075 | 1.0464 | 1 | 1 |
| WCM0_LM2 | 2 | 242021730 | 242815417 | 0.1983 | −0.1009 | 0.9529 | 0.912 | 1 | 1 |
| WCM0_LM2 | 20 | 68427 | 6021946 | −0.1052 | −0.4195 | 1.0074 | 0.488 | 1 | 0 |
| WCM0_LM2 | 20 | 6022157 | 25284255 | 0.0632 | −0.2423 | 1.1355 | 0.5553 | 1 | 1 |
| WCM0_LM2 | 20 | 25287535 | 34317470 | −0.1843 | −0.5032 | 0.9013 | 0.5098 | 1 | 1 |
| WCM0_LM2 | 20 | 34319996 | 35646733 | 0.0576 | −0.2482 | NA | NA | NA | NA |
| WCM0_LM2 | 20 | 35649104 | 35717534 | −0.1287 | −0.4443 | NA | NA | NA | NA |
| WCM0_LM2 | 20 | 35724166 | 45771777 | 0.3337 | 0.0403 | 1.0294 | 1.0272 | 1 | 1 |
| WCM0_LM2 | 20 | 45797821 | 55213077 | 0.1817 | −0.1182 | 0.9291 | 0.9136 | 1 | 1 |
| WCM0_LM2 | 20 | 55746069 | 62076743 | 0.4089 | 0.1185 | 1.0912 | 1.0799 | 1 | 1 |
| WCM0_LM2 | 20 | 62078160 | 62680301 | 0.1698 | −0.1307 | 0.9229 | 0.9038 | 1 | 1 |
| WCM0_LM2 | 20 | 62680599 | 62904890 | 0.4575 | 0.1689 | NA | NA | NA | NA |
| WCM0_LM2 | 21 | 10862618 | 34400082 | 0.3411 | 0.048 | 1.0403 | 1.0274 | 1 | 1 |
| WCM0_LM2 | 21 | 34442624 | 36081024 | 0.2171 | −0.0812 | 0.961 | 0.9295 | 1 | 1 |
| WCM0_LM2 | 21 | 36081626 | 37518362 | −0.5467 | −0.8901 | NA | NA | NA | NA |
| WCM0_LM2 | 21 | 37518791 | 37781756 | 0.0685 | −0.2368 | NA | NA | NA | NA |
| WCM0_LM2 | 21 | 37783854 | 48084277 | 0.3659 | 0.0738 | 1.0568 | 1.0481 | 1 | 1 |
| WCM0_LM2 | 22 | 16255826 | 28310267 | −0.6349 | −0.9854 | 1.0136 | −0.0034 | 1 | 0 |
| WCM0_LM2 | 22 | 28315195 | 43575733 | −0.7425 | −1.1023 | 0.9428 | −0.0112 | 1 | 0 |
| WCM0_LM2 | 22 | 43576012 | 51216382 | −0.6547 | −1.0068 | 0.9992 | −0.0039 | 1 | 0 |
| WCM0_LM2 | 3 | 361508 | 9426194 | −0.5228 | −0.8643 | 1.0983 | 3.00E−04 | 1 | 0 |
| WCM0_LM2 | 3 | 9470717 | 10360847 | −0.7534 | −1.1141 | 0.9258 | −0.0019 | 1 | 0 |
| WCM0_LM2 | 3 | 10362734 | 47088052 | −0.5503 | −0.894 | 1.0773 | −0.0011 | 1 | 0 |
| WCM0_LM2 | 3 | 47098357 | 50211269 | −0.7599 | −1.1212 | 0.9295 | −0.0101 | 1 | 0 |
| WCM0_LM2 | 3 | 50211641 | 56600977 | −0.5628 | −0.9075 | 1.0677 | −0.0014 | 1 | 0 |
| WCM0_LM2 | 3 | 56605328 | 58355222 | −0.7496 | −1.11 | 0.9339 | −0.0073 | 1 | 0 |
| WCM0_LM2 | 3 | 58368449 | 73437085 | −0.5284 | −0.8705 | 1.0968 | −0.0028 | 1 | 0 |
| WCM0_LM2 | 3 | 73439038 | 73673935 | −0.3539 | −0.6835 | NA | NA | NA | NA |
| WCM0_LM2 | 3 | 74313609 | 75986686 | 0.9613 | 0.6884 | NA | NA | NA | NA |
| WCM0_LM2 | 3 | 77089935 | 93780313 | 0.2263 | −0.0716 | NA | NA | NA | NA |
| WCM0_LM2 | 3 | 93782011 | 122103070 | −0.5639 | −0.9087 | 1.0668 | −0.0015 | 1 | 0 |
| WCM0_LM2 | 3 | 122121686 | 122419305 | 0.2749 | −0.0209 | NA | NA | NA | NA |
| WCM0_LM2 | 3 | 122419676 | 134968372 | 0.3893 | 0.0981 | 1.0748 | 1.0659 | 1 | 1 |
| WCM0_LM2 | 3 | 134977941 | 138669417 | 0.2068 | −0.092 | NA | NA | NA | NA |
| WCM0_LM2 | 3 | 138738875 | 141231057 | 0.5137 | 0.2271 | NA | NA | NA | NA |
| WCM0_LM2 | 3 | 141235302 | 142681100 | 0.1721 | −0.1282 | 0.915 | 0.915 | 1 | 1 |
| WCM0_LM2 | 3 | 142681387 | 169485905 | 0.3913 | 0.1002 | 1.0753 | 1.0686 | 1 | 1 |
| WCM0_LM2 | 3 | 169486144 | 186760820 | 0.2448 | −0.0524 | 0.9674 | 0.9613 | 1 | 1 |
| WCM0_LM2 | 3 | 186761066 | 194126811 | 0.4204 | 0.1304 | 1.1003 | 1.0889 | 1 | 1 |
| WCM0_LM2 | 3 | 194134535 | 197847254 | 0.1567 | −0.1443 | 0.9133 | 0.8963 | 1 | 1 |
| WCM0_LM2 | 4 | 53424 | 3213848 | −0.2337 | −0.5556 | 0.8299 | 0.5309 | 1 | 1 |
| WCM0_LM2 | 4 | 3214350 | 38775142 | −0.0488 | −0.3601 | 1.0032 | 0.555 | 1 | 1 |
| WCM0_LM2 | 4 | 38775350 | 39201204 | −0.2034 | −0.5234 | 0.855 | 0.5364 | 1 | 1 |
| WCM0_LM2 | 4 | 39205259 | 40832484 | −0.4446 | −0.7805 | 0.7388 | 0.4256 | 1 | 0 |
| WCM0_LM2 | 4 | 40892531 | 41035318 | −1.6125 | −2.0832 | NA | NA | NA | NA |
| WCM0_LM2 | 4 | 41259149 | 41362962 | −0.2233 | −0.5445 | NA | NA | NA | NA |
| WCM0_LM2 | 4 | 41496568 | 44631587 | −1.4169 | −1.8557 | 0.544 | 0.0086 | 1 | 0 |
| WCM0_LM2 | 4 | 44638048 | 65147257 | −0.066 | −0.3782 | 1.0241 | 0.5147 | 1 | 1 |
| WCM0_LM2 | 4 | 65155423 | 71503580 | 0.3344 | 0.041 | 1.0353 | 1.0224 | 1 | 1 |
| WCM0_LM2 | 4 | 71507712 | 71895093 | 0.0571 | −0.2487 | NA | NA | NA | NA |
| WCM0_LM2 | 4 | 72102288 | 73924001 | −0.5136 | −0.8545 | NA | NA | NA | NA |
| WCM0_LM2 | 4 | 73927521 | 106158303 | 0.3111 | 0.0168 | 1.0159 | 1.0075 | 1 | 1 |
| WCM0_LM2 | 4 | 106158545 | 106474000 | −0.5826 | −0.9288 | NA | NA | NA | NA |
| WCM0_LM2 | 4 | 106510551 | 174537763 | 0.3142 | 0.02 | 1.0224 | 1.0055 | 1 | 1 |
| WCM0_LM2 | 4 | 175158641 | 177084265 | −0.0119 | −0.3212 | NA | NA | NA | NA |
| WCM0_LM2 | 4 | 177087286 | 178911657 | −0.695 | −1.0506 | NA | NA | NA | NA |
| WCM0_LM2 | 4 | 183090436 | 190947560 | −0.098 | −0.4119 | 0.988 | 0.5152 | 1 | 1 |
| WCM0_LM2 | 5 | 140681 | 192214 | 0.1443 | −0.1573 | NA | NA | NA | NA |
| WCM0_LM2 | 5 | 192445 | 1495038 | −0.0356 | −0.3462 | 1.0327 | 0.5407 | 1 | 1 |
| WCM0_LM2 | 5 | 1501550 | 16479110 | 0.0886 | −0.2157 | 1.1428 | 0.5795 | 1 | 1 |
| WCM0_LM2 | 5 | 16481185 | 88416361 | −0.0686 | −0.381 | 1.0108 | 0.525 | 1 | 1 |
| WCM0_LM2 | 5 | 89689893 | 122950042 | 0.3547 | 0.0622 | 1.0516 | 1.0364 | 1 | 1 |
| WCM0_LM2 | 5 | 123966403 | 123983184 | −0.34 | −0.6687 | NA | NA | NA | NA |
| WCM0_LM2 | 5 | 123983401 | 126171922 | −0.7772 | −1.1401 | NA | NA | NA | NA |
| WCM0_LM2 | 5 | 126206303 | 132198287 | −0.5375 | −0.8802 | 1.0873 | −7.00E−04 | 1 | 0 |
| WCM0_LM2 | 5 | 132199874 | 134369377 | −0.7657 | −1.1275 | 0.9161 | −7.00E−04 | 1 | 0 |
| WCM0_LM2 | 5 | 134670765 | 136957765 | −0.3381 | −0.6666 | 1.2305 | 0.0295 | 1 | 0 |
| WCM0_LM2 | 5 | 136961471 | 137661766 | 0.0899 | −0.2143 | NA | NA | NA | NA |
| WCM0_LM2 | 5 | 137664235 | 140174634 | 0.1865 | −0.1132 | 0.9361 | 0.9129 | 1 | 1 |
| WCM0_LM2 | 5 | 140174822 | 170323125 | 0.341 | 0.0479 | 1.0381 | 1.0295 | 1 | 1 |
| WCM0_LM2 | 5 | 170336668 | 180687812 | 0.2071 | −0.0916 | 0.9443 | 0.9326 | 1 | 1 |
| WCM0_LM2 | 6 | 292598 | 38650588 | 0.2967 | 0.0017 | 1.0043 | 0.9981 | 1 | 1 |
| WCM0_LM2 | 6 | 38652308 | 41318641 | 0.4763 | 0.1884 | 1.1655 | 1.1135 | 1 | 1 |
| WCM0_LM2 | 6 | 41533685 | 43639680 | 0.2066 | −0.0922 | 0.939 | 0.9371 | 1 | 1 |
| WCM0_LM2 | 6 | 43642929 | 56765338 | 0.4379 | 0.1485 | 1.1165 | 1.1004 | 1 | 1 |
| WCM0_LM2 | 6 | 56818622 | 91296463 | 0.2867 | −0.0086 | 1.0614 | 0.9267 | 1 | 1 |
| WCM0_LM2 | 6 | 93953242 | 97730322 | 0.0422 | −0.2643 | NA | NA | NA | NA |
| WCM0_LM2 | 6 | 99282794 | 118972442 | 0.36 | 0.0677 | 1.0611 | 1.0349 | 1 | 1 |
| WCM0_LM2 | 6 | 119222019 | 121768849 | −0.0502 | −0.3615 | NA | NA | NA | NA |
| WCM0_LM2 | 6 | 122720983 | 124979510 | 0.4086 | 0.1182 | NA | NA | NA | NA |
| WCM0_LM2 | 6 | 125112467 | 149395270 | −0.0055 | −0.3145 | 1.0824 | 0.5259 | 1 | 1 |
| WCM0_LM2 | 6 | 149691155 | 151939210 | −0.1888 | −0.5079 | 0.9684 | 0.4381 | 1 | 0 |
| WCM0_LM2 | 6 | 152129246 | 166755979 | −0.0401 | −0.3509 | 1.0368 | 0.5314 | 1 | 1 |
| WCM0_LM2 | 6 | 166778877 | 170893364 | 0.4645 | 0.1762 | 1.1592 | 1.1006 | 1 | 1 |
| WCM0_LM2 | 7 | 193578 | 4900132 | 0.2325 | −0.0652 | 0.9648 | 0.9468 | 1 | 1 |
| WCM0_LM2 | 7 | 4900442 | 7118626 | 0.0181 | −0.2897 | 0.8387 | 0.7974 | 1 | 1 |
| WCM0_LM2 | 7 | 7274094 | 22771138 | 0.4337 | 0.1442 | 1.1109 | 1.0994 | 1 | 1 |
| WCM0_LM2 | 7 | 22852843 | 27196068 | 0.282 | −0.0136 | 1.0133 | 0.9679 | 1 | 1 |
| WCM0_LM2 | 7 | 27203339 | 27689282 | −0.5128 | −0.8536 | NA | NA | NA | NA |
| WCM0_LM2 | 7 | 27702390 | 29160564 | 0.4545 | 0.1658 | NA | NA | NA | NA |
| WCM0_LM2 | 7 | 29394264 | 38398377 | −0.482 | −0.8205 | 1.1352 | −0.0028 | 1 | 0 |
| WCM0_LM2 | 7 | 38402486 | 55565332 | 0.3784 | 0.0868 | 1.073 | 1.0509 | 1 | 1 |
| WCM0_LM2 | 7 | 55588756 | 77755197 | 0.0987 | −0.205 | 0.8748 | 0.8602 | 1 | 1 |
| WCM0_LM2 | 7 | 77756649 | 97364091 | 0.4001 | 0.1094 | 1.081 | 1.0765 | 1 | 1 |
| WCM0_LM2 | 7 | 97365643 | 102572301 | 0.0895 | −0.2148 | 0.9764 | 0.747 | 1 | 1 |
| WCM0_LM2 | 7 | 102574422 | 107688446 | 0.2526 | −0.0442 | 0.98 | 0.9597 | 1 | 1 |
| WCM0_LM2 | 7 | 107688571 | 127847987 | 0.4056 | 0.1151 | 1.0931 | 1.0729 | 1 | 1 |
| WCM0_LM2 | 7 | 127892030 | 137600663 | 0.2861 | −0.0093 | 0.9988 | 0.9884 | 1 | 1 |
| WCM0_LM2 | 7 | 137613080 | 140710306 | 0.151 | −0.1504 | 0.9039 | 0.8982 | 1 | 1 |
| WCM0_LM2 | 7 | 141255364 | 158937481 | 0.3637 | 0.0714 | 1.0573 | 1.0442 | 1 | 1 |
| WCM0_LM2 | 8 | 192902 | 2092907 | −4.0684 | −6.8052 | NA | NA | NA | NA |
| WCM0_LM2 | 8 | 2796243 | 36703425 | −0.4687 | −0.8062 | 1.1486 | −0.0049 | 1 | 0 |
| WCM0_LM2 | 8 | 36722007 | 37686408 | 0.4284 | 0.1387 | NA | NA | NA | NA |
| WCM0_LM2 | 8 | 37686742 | 49641670 | 0.2568 | −0.0398 | 0.9951 | 0.9505 | 1 | 1 |
| WCM0_LM2 | 8 | 49642417 | 67372731 | 0.4412 | 0.152 | 1.1261 | 1.0961 | 1 | 1 |
| WCM0_LM2 | 8 | 67380528 | 68423840 | 0.1469 | −0.1546 | NA | NA | NA | NA |
| WCM0_LM2 | 8 | 68430319 | 105601105 | 0.4061 | 0.1155 | 1.0901 | 1.0766 | 1 | 1 |
| WCM0_LM2 | 8 | 106430834 | 131249154 | −0.4665 | −0.8039 | 1.1491 | −0.0036 | 1 | 0 |
| WCM0_LM2 | 8 | 131370259 | 140715014 | −0.3087 | −0.6353 | 1.2758 | 0.0118 | 1 | 0 |
| WCM0_LM2 | 8 | 140715222 | 146279476 | −0.5106 | −0.8512 | 1.1133 | −0.0047 | 1 | 0 |
| WCM0_LM2 | 9 | 117666 | 3395520 | −1.4882 | −1.938 | 0.5264 | −0.0044 | 1 | 0 |
| WCM0_LM2 | 9 | 3828232 | 19032907 | 0.0174 | −0.2904 | 1.1115 | 0.5238 | 1 | 1 |
| WCM0_LM2 | 9 | 19049792 | 40772506 | −0.5509 | −0.8947 | 1.0879 | −0.0121 | 1 | 0 |
| WCM0_LM2 | 9 | 40773337 | 99416976 | −0.0973 | −0.4112 | 1.0663 | 0.4377 | 1 | 0 |
| WCM0_LM2 | 9 | 99521023 | 114090390 | 0.3523 | 0.0596 | 1.0476 | 1.0368 | 1 | 1 |
| WCM0_LM2 | 9 | 114090601 | 116122975 | 0.1592 | −0.1418 | 0.9216 | 0.8912 | 1 | 1 |
| WCM0_LM2 | 9 | 116124604 | 116303584 | 0.3053 | 0.0107 | NA | NA | NA | NA |
| WCM0_LM2 | 9 | 116345746 | 123230157 | 0.4729 | 0.1848 | 1.1426 | 1.1308 | 1 | 1 |
| WCM0_LM2 | 9 | 123232405 | 127300561 | 0.3197 | 0.0257 | 1.0254 | 1.0105 | 1 | 1 |
| WCM0_LM2 | 9 | 127302394 | 135702448 | 0.1742 | −0.1261 | 0.9192 | 0.9134 | 1 | 1 |
| WCM0_LM2 | 9 | 135703440 | 141071438 | 0.3232 | 0.0294 | 1.0238 | 1.0174 | 1 | 1 |
| WCM0_LM2 | X | 2700140 | 5950784 | 0.2095 | −0.0892 | NA | NA | NA | NA |
| WCM0_LM2 | X | 6069121 | 15527472 | 0.4174 | 0.1273 | NA | NA | NA | NA |
| WCM0_LM2 | X | 15529580 | 100662726 | 0.2467 | −0.0504 | NA | NA | NA | NA |
| WCM0_LM2 | X | 100808315 | 112034033 | 0.442 | 0.1528 | NA | NA | NA | NA |
| WCM0_LM2 | X | 112035075 | 125299467 | 0.1779 | −0.1222 | NA | NA | NA | NA |
| WCM0_LM2 | X | 125299645 | 154509348 | 0.8717 | 0.5963 | NA | NA | NA | NA |
| WCM0_PM1 | 1 | 808999 | 2560735 | 0.1589 | −0.1421 | 0.9109 | 0.9015 | 1 | 1 |
| WCM0_PM1 | 1 | 2938320 | 6219610 | 0.3758 | 0.084 | 1.0732 | 1.0468 | 1 | 1 |
| WCM0_PM1 | 1 | 6228265 | 11986004 | 0.1131 | −0.19 | 0.8812 | 0.872 | 1 | 1 |
| WCM0_PM1 | 1 | 11994910 | 17950850 | −0.7599 | −1.1212 | 0.921 | −0.0016 | 1 | 0 |
| WCM0_PM1 | 1 | 17952432 | 19448407 | −0.4833 | −0.822 | 1.1311 | 2.00E−04 | 1 | 0 |
| WCM0_PM1 | 1 | 19449351 | 19609254 | −0.7176 | −1.0751 | 0.959 | −0.0097 | 1 | 0 |
| WCM0_PM1 | 1 | 19611241 | 27755381 | −0.0152 | −0.3247 | 0.9144 | 0.6825 | 1 | 1 |
| WCM0_PM1 | 1 | 27874007 | 27878413 | 1.7295 | 1.4718 | NA | NA | NA | NA |
| WCM0_PM1 | 1 | 27939619 | 32280238 | 0.7136 | 0.4337 | 1.7252 | 0.9761 | 2 | 1 |
| WCM0_PM1 | 1 | 32280311 | 33557797 | 0.4236 | 0.1337 | 1.4308 | 0.7634 | 1 | 1 |
| WCM0_PM1 | 1 | 33558938 | 33613329 | 0.6532 | 0.3714 | NA | NA | NA | NA |
| WCM0_PM1 | 1 | 33623966 | 35370875 | 0.9375 | 0.664 | NA | NA | NA | NA |
| WCM0_PM1 | 1 | 35452804 | 41013005 | 0.5352 | 0.2494 | 1.4483 | 0.9291 | 1 | 1 |
| WCM0_PM1 | 1 | 41092196 | 45205685 | 0.6929 | 0.4123 | 1.5518 | 1.1099 | 2 | 1 |
| WCM0_PM1 | 1 | 45206676 | 45243625 | 0.6085 | 0.3252 | NA | NA | NA | NA |
| WCM0_PM1 | 1 | 45243741 | 51778500 | 0.503 | 0.216 | 1.3492 | 0.9738 | 1 | 1 |
| WCM0_PM1 | 1 | 51787496 | 53516315 | 0.27 | −0.026 | NA | NA | NA | NA |
| WCM0_PM1 | 1 | 53527948 | 61872414 | 0.5734 | 0.2889 | 1.3759 | 1.0676 | 1 | 1 |
| WCM0_PM1 | 1 | 61920994 | 64011682 | 0.2325 | −0.0652 | 0.9957 | 0.9159 | 1 | 1 |
| WCM0_PM1 | 1 | 64017382 | 90486493 | 0.5182 | 0.2318 | 1.1962 | 1.1524 | 1 | 1 |
| WCM0_PM1 | 1 | 90487798 | 91182716 | 0.8044 | 0.5272 | NA | NA | NA | NA |
| WCM0_PM1 | 1 | 91382423 | 94018018 | −0.712 | −1.0691 | 0.9524 | 8.00E−04 | 1 | 0 |
| WCM0_PM1 | 1 | 94027772 | 109171074 | −0.4489 | −0.785 | 1.1563 | 0.0044 | 1 | 0 |
| WCM0_PM1 | 1 | 109171370 | 109271473 | −0.6294 | −0.9795 | NA | NA | NA | NA |
| WCM0_PM1 | 1 | 109273311 | 109964463 | −0.7224 | −1.0803 | 0.9433 | 0.0026 | 1 | 0 |
| WCM0_PM1 | 1 | 109968935 | 112991698 | −0.4264 | −0.761 | 1.1701 | 0.0101 | 1 | 0 |
| WCM0_PM1 | 1 | 112991790 | 115576719 | −0.6305 | −0.9807 | 1.0053 | 0.0082 | 1 | 0 |
| WCM0_PM1 | 1 | 115576823 | 119935289 | −0.4003 | −0.733 | 1.2038 | −5.00E−04 | 1 | 0 |
| WCM0_PM1 | 1 | 119936435 | 145115810 | −0.2783 | −0.6029 | 1.0617 | 0.2552 | 1 | 0 |
| WCM0_PM1 | 1 | 145139406 | 149921981 | −0.1009 | −0.415 | 1.031 | 0.469 | 1 | 0 |
| WCM0_PM1 | 1 | 149931698 | 152079989 | −0.3517 | −0.6811 | 0.7867 | 0.4607 | 1 | 0 |
| WCM0_PM1 | 1 | 152080243 | 153391752 | 0.0359 | −0.271 | 0.9532 | 0.7043 | 1 | 1 |
| WCM0_PM1 | 1 | 153409657 | 153580668 | −0.331 | −0.659 | NA | NA | NA | NA |
| WCM0_PM1 | 1 | 153587435 | 155173361 | −0.4962 | −0.8358 | 0.6572 | 0.4634 | 1 | 0 |
| WCM0_PM1 | 1 | 155174667 | 156339241 | −0.6111 | −0.9597 | 0.6498 | 0.3786 | 1 | 0 |
| WCM0_PM1 | 1 | 156347289 | 156873714 | −0.396 | −0.7284 | 0.703 | 0.5042 | 1 | 1 |
| WCM0_PM1 | 1 | 156874512 | 160786624 | −0.0932 | −0.4069 | 0.8872 | 0.6213 | 1 | 1 |
| WCM0_PM1 | 1 | 160788089 | 161487989 | −0.463 | −0.8002 | NA | NA | NA | NA |
| WCM0_PM1 | 1 | 161494909 | 173490418 | −0.1465 | −0.4631 | 0.8512 | 0.5996 | 1 | 1 |
| WCM0_PM1 | 1 | 173493205 | 174976273 | −0.3572 | −0.6869 | NA | NA | NA | NA |
| WCM0_PM1 | 1 | 174977786 | 178745891 | −0.0081 | −0.3172 | 0.9546 | 0.6506 | 1 | 1 |
| WCM0_PM1 | 1 | 178753657 | 180144472 | −0.4173 | −0.7512 | 0.6431 | 0.5451 | 1 | 1 |
| WCM0_PM1 | 1 | 180145155 | 180832953 | −0.1711 | −0.4892 | NA | NA | NA | NA |
| WCM0_PM1 | 1 | 180842940 | 198828281 | 0.118 | −0.1849 | 1.1755 | 0.584 | 1 | 1 |
| WCM0_PM1 | 1 | 199997023 | 205890946 | −0.057 | −0.3687 | 1.0307 | 0.5182 | 1 | 1 |
| WCM0_PM1 | 1 | 205892303 | 211263914 | 0.1704 | −0.1301 | 1.188 | 0.6396 | 1 | 1 |
| WCM0_PM1 | 1 | 211276849 | 213445936 | −0.1157 | −0.4306 | 0.9574 | 0.5265 | 1 | 1 |
| WCM0_PM1 | 1 | 214169997 | 223986386 | 0.1257 | −0.1768 | 1.183 | 0.5864 | 1 | 1 |
| WCM0_PM1 | 1 | 223987701 | 226567838 | −0.1787 | −0.4973 | 0.911 | 0.5059 | 1 | 1 |
| WCM0_PM1 | 1 | 226568860 | 233231604 | 0.2365 | −0.061 | 0.9756 | 0.9416 | 1 | 1 |
| WCM0_PM1 | 1 | 233270956 | 233394314 | −0.6191 | −0.9683 | NA | NA | NA | NA |
| WCM0_PM1 | 1 | 233394739 | 234745243 | 0.2638 | −0.0326 | 1.0042 | 0.9512 | 1 | 1 |
| WCM0_PM1 | 1 | 235277191 | 236767444 | −0.049 | −0.3603 | 0.7922 | 0.7659 | 1 | 1 |
| WCM0_PM1 | 1 | 236849945 | 240256822 | 0.2715 | −0.0245 | 1.0282 | 0.9381 | 1 | 1 |
| WCM0_PM1 | 1 | 240256995 | 247492594 | 0.1094 | −0.1939 | 0.8907 | 0.8577 | 1 | 1 |
| WCM0_PM1 | 1 | 247492845 | 249212577 | 0.4072 | 0.1167 | 1.1833 | 0.9852 | 1 | 1 |
| WCM0_PM1 | 10 | 226030 | 5454638 | −0.4128 | −0.7464 | 1.1822 | 0.01 | 1 | 0 |
| WCM0_PM1 | 10 | 5468674 | 44868750 | −0.6676 | −1.0208 | 0.9846 | 0.0011 | 1 | 0 |
| WCM0_PM1 | 10 | 44871415 | 64911893 | −0.4201 | −0.7542 | 1.1811 | 0.0047 | 1 | 0 |
| WCM0_PM1 | 10 | 64927891 | 65928961 | −0.8733 | −1.2453 | NA | NA | NA | NA |
| WCM0_PM1 | 10 | 65929410 | 71078775 | −0.9366 | −1.315 | 0.8087 | −0.0048 | 1 | 0 |
| WCM0_PM1 | 10 | 71103597 | 73767859 | −0.5316 | −0.8739 | 1.0825 | 0.0088 | 1 | 0 |
| WCM0_PM1 | 10 | 73768169 | 76989406 | −0.7844 | −1.1479 | 0.9088 | −0.0062 | 1 | 0 |
| WCM0_PM1 | 10 | 76993786 | 92982637 | −0.4679 | −0.8055 | 1.1431 | 0.0013 | 1 | 0 |
| WCM0_PM1 | 10 | 93000258 | 99527563 | −0.6746 | −1.0284 | 0.9833 | −0.0028 | 1 | 0 |
| WCM0_PM1 | 10 | 99529499 | 101478074 | −0.4314 | −0.7663 | 1.1558 | 0.02 | 1 | 0 |
| WCM0_PM1 | 10 | 101478296 | 105765735 | −0.7684 | −1.1305 | 0.9203 | −0.0068 | 1 | 0 |
| WCM0_PM1 | 10 | 105768114 | 120354668 | −0.4948 | −0.8342 | 1.1092 | 0.0126 | 1 | 0 |
| WCM0_PM1 | 10 | 120445630 | 121678041 | −0.7449 | −1.1048 | NA | NA | NA | NA |
| WCM0_PM1 | 10 | 121679118 | 135373653 | −0.4742 | −0.8121 | 1.1374 | 0.0017 | 1 | 0 |
| WCM0_PM1 | 11 | 193096 | 1958179 | −0.7117 | −1.0687 | 0.9428 | 0.0107 | 1 | 0 |
| WCM0_PM1 | 11 | 1959656 | 4389433 | −0.5249 | −0.8667 | 1.0895 | 0.0073 | 1 | 0 |
| WCM0_PM1 | 11 | 4406533 | 8118414 | −0.2863 | −0.6115 | 1.295 | 0.014 | 1 | 0 |
| WCM0_PM1 | 11 | 8118740 | 8950950 | −0.4816 | −0.8201 | NA | NA | NA | NA |
| WCM0_PM1 | 11 | 8953687 | 10215506 | −0.7355 | −1.0946 | 0.9415 | −0.005 | 1 | 0 |
| WCM0_PM1 | 11 | 10327290 | 18048219 | −0.3754 | −0.7064 | 1.2196 | 0.0061 | 1 | 0 |
| WCM0_PM1 | 11 | 18050907 | 18722791 | −0.6415 | −0.9925 | 1.0305 | −0.0253 | 1 | 0 |
| WCM0_PM1 | 11 | 18723243 | 32605410 | −0.3446 | −0.6735 | 1.2408 | 0.0132 | 1 | 0 |
| WCM0_PM1 | 11 | 32608487 | 34533145 | −0.6805 | −1.0348 | 0.9717 | 0.0044 | 1 | 0 |
| WCM0_PM1 | 11 | 34664274 | 45967621 | −0.3727 | −0.7036 | 1.2199 | 0.0082 | 1 | 0 |
| WCM0_PM1 | 11 | 45970395 | 48632382 | −0.8284 | −1.196 | 0.8706 | 0.0023 | 1 | 0 |
| WCM0_PM1 | 11 | 49059138 | 61049400 | −0.4925 | −0.8319 | 1.111 | 0.0126 | 1 | 0 |
| WCM0_PM1 | 11 | 61050414 | 69488012 | −0.7674 | −1.1294 | 0.921 | −0.0068 | 1 | 0 |
| WCM0_PM1 | 11 | 69489942 | 78279790 | 0.3203 | 0.0263 | 1.0237 | 1.0131 | 1 | 1 |
| WCM0_PM1 | 11 | 78282405 | 107288907 | 0.4611 | 0.1726 | 1.1282 | 1.1259 | 1 | 1 |
| WCM0_PM1 | 11 | 107299631 | 108412378 | 0.1469 | −0.1546 | 0.8984 | 0.8984 | 1 | 1 |
| WCM0_PM1 | 11 | 108464103 | 116798133 | 0.4092 | 0.1188 | 1.1027 | 1.0689 | 1 | 1 |
| WCM0_PM1 | 11 | 116824796 | 117117528 | −0.7115 | −1.0685 | NA | NA | NA | NA |
| WCM0_PM1 | 11 | 117150646 | 118656889 | 0.1888 | −0.1108 | 0.9677 | 0.8844 | 1 | 1 |
| WCM0_PM1 | 11 | 118754631 | 119005066 | −0.807 | −1.1726 | NA | NA | NA | NA |
| WCM0_PM1 | 11 | 119020704 | 119180616 | 0.2753 | −0.0205 | NA | NA | NA | NA |
| WCM0_PM1 | 11 | 119181170 | 134257494 | 0.4804 | 0.1926 | 1.1464 | 1.1393 | 1 | 1 |
| WCM0_PM1 | 12 | 247582 | 6219763 | −0.5051 | −0.8453 | 1.1124 | 8.00E−04 | 1 | 0 |
| WCM0_PM1 | 12 | 6220067 | 9070366 | −0.7476 | −1.1078 | 0.9282 | −2.00E−04 | 1 | 0 |
| WCM0_PM1 | 12 | 9072399 | 9459772 | −0.5273 | −0.8692 | NA | NA | NA | NA |
| WCM0_PM1 | 12 | 9464146 | 9723417 | 0.2518 | −0.045 | NA | NA | NA | NA |
| WCM0_PM1 | 12 | 9747833 | 25348041 | −0.4949 | −0.8343 | 1.1025 | 0.0192 | 1 | 0 |
| WCM0_PM1 | 12 | 25356953 | 31285255 | 0.3909 | 0.0998 | 1.0927 | 1.0504 | 1 | 1 |
| WCM0_PM1 | 12 | 31288604 | 32975453 | 0.0615 | −0.2441 | 0.8718 | 0.8169 | 1 | 1 |
| WCM0_PM1 | 12 | 32976987 | 41587946 | 0.3992 | 0.1084 | NA | NA | NA | NA |
| WCM0_PM1 | 12 | 41831837 | 42840394 | −0.6211 | −0.9704 | NA | NA | NA | NA |
| WCM0_PM1 | 12 | 42853638 | 48741912 | −0.45 | −0.7862 | 1.1417 | 0.018 | 1 | 0 |
| WCM0_PM1 | 12 | 48743865 | 52200100 | −0.8023 | −1.1675 | 0.8965 | −0.0061 | 1 | 0 |
| WCM0_PM1 | 12 | 52200459 | 53295827 | −0.4489 | −0.785 | 1.152 | 0.0087 | 1 | 0 |
| WCM0_PM1 | 12 | 53298468 | 54070021 | −0.8524 | −1.2224 | 0.864 | −0.0069 | 1 | 0 |
| WCM0_PM1 | 12 | 54105819 | 56142337 | −0.5704 | −0.9157 | NA | NA | NA | NA |
| WCM0_PM1 | 12 | 56142662 | 57110231 | −0.8764 | −1.2487 | NA | NA | NA | NA |
| WCM0_PM1 | 12 | 57110604 | 57995392 | −0.67 | −1.0234 | 0.9914 | −0.0075 | 1 | 0 |
| WCM0_PM1 | 12 | 58000350 | 77260012 | −0.5494 | −0.893 | 1.0705 | 0.0065 | 1 | 0 |
| WCM0_PM1 | 12 | 77417806 | 93169949 | −0.4116 | −0.7451 | 1.1884 | 0.0049 | 1 | 0 |
| WCM0_PM1 | 12 | 93170703 | 94676118 | −0.62 | −0.9692 | NA | NA | NA | NA |
| WCM0_PM1 | 12 | 94688232 | 102224465 | −0.6893 | −1.0444 | 0.9782 | −0.0085 | 1 | 0 |
| WCM0_PM1 | 12 | 102271729 | 108919881 | −0.4974 | −0.837 | 1.1179 | 0.0016 | 1 | 0 |
| WCM0_PM1 | 12 | 108920216 | 110403609 | −0.7161 | −1.0735 | 0.9533 | −0.0029 | 1 | 0 |
| WCM0_PM1 | 12 | 110405149 | 112926987 | −0.916 | −1.2923 | NA | NA | NA | NA |
| WCM0_PM1 | 12 | 112942618 | 120536922 | −0.6441 | −0.9954 | 1.0063 | −0.0031 | 1 | 0 |
| WCM0_PM1 | 12 | 120541618 | 124175252 | −0.9175 | −1.294 | 0.829 | −0.0133 | 1 | 0 |
| WCM0_PM1 | 12 | 124177196 | 133779197 | −0.6301 | −0.9801 | 1.0155 | −0.0016 | 1 | 0 |
| WCM0_PM1 | 13 | 19705470 | 19969223 | −0.4687 | −0.8062 | NA | NA | NA | NA |
| WCM0_PM1 | 13 | 19972029 | 23915758 | −0.8061 | −1.1716 | 0.8839 | 0.004 | 1 | 0 |
| WCM0_PM1 | 13 | 23927969 | 80911785 | −0.5224 | −0.8639 | 1.0921 | 0.0068 | 1 | 0 |
| WCM0_PM1 | 13 | 84453738 | 95686877 | 0.5658 | 0.281 | NA | NA | NA | NA |
| WCM0_PM1 | 13 | 95695990 | 101287231 | 0.3267 | 0.033 | 2.0517 | −0.0055 | 2 | 0 |
| WCM0_PM1 | 13 | 101287365 | 113158944 | 0.5459 | 0.2604 | 2.3878 | 0.0079 | 2 | 0 |
| WCM0_PM1 | 13 | 113170707 | 114498140 | 0.3542 | 0.0616 | 2.0812 | 0.0061 | 2 | 0 |
| WCM0_PM1 | 13 | 114502382 | 115089411 | 0.6008 | 0.3173 | 2.4744 | 0.0176 | 2 | 0 |
| WCM0_PM1 | 14 | 19553749 | 20692044 | 0.4177 | 0.1276 | 1.1015 | 1.0834 | 1 | 1 |
| WCM0_PM1 | 14 | 20692188 | 22134203 | 0.2126 | −0.086 | 0.9451 | 0.9392 | 1 | 1 |
| WCM0_PM1 | 14 | 22167282 | 23236528 | 0.4945 | 0.2073 | 1.1772 | 1.1318 | 1 | 1 |
| WCM0_PM1 | 14 | 23237175 | 23791368 | 0.104 | −0.1996 | 0.9219 | 0.8197 | 1 | 1 |
| WCM0_PM1 | 14 | 23792215 | 31598638 | 0.3818 | 0.0904 | 1.0713 | 1.058 | 1 | 1 |
| WCM0_PM1 | 14 | 31602596 | 34420005 | 0.0394 | −0.2673 | NA | NA | NA | NA |
| WCM0_PM1 | 14 | 34904445 | 36231940 | −0.4437 | −0.7795 | NA | NA | NA | NA |
| WCM0_PM1 | 14 | 36240179 | 47600987 | −0.0726 | −0.3851 | 1.0305 | 0.5009 | 1 | 1 |
| WCM0_PM1 | 14 | 50044532 | 50971559 | −0.3225 | −0.65 | NA | NA | NA | NA |
| WCM0_PM1 | 14 | 50998664 | 63784574 | −0.0867 | −0.4 | 1.0033 | 0.5124 | 1 | 1 |
| WCM0_PM1 | 14 | 63842768 | 65007733 | −0.3112 | −0.638 | NA | NA | NA | NA |
| WCM0_PM1 | 14 | 65007871 | 73441696 | −0.0684 | −0.3807 | 1.0255 | 0.5106 | 1 | 1 |
| WCM0_PM1 | 14 | 73442442 | 75748069 | −0.3397 | −0.6684 | 0.8265 | 0.432 | 1 | 0 |
| WCM0_PM1 | 14 | 75904650 | 77242347 | −0.0572 | −0.3689 | NA | NA | NA | NA |
| WCM0_PM1 | 14 | 77242535 | 78365565 | −0.2781 | −0.6027 | 0.8735 | 0.4436 | 1 | 0 |
| WCM0_PM1 | 14 | 78374254 | 86089315 | 0.0558 | −0.2501 | NA | NA | NA | NA |
| WCM0_PM1 | 14 | 86089357 | 89082517 | 0.367 | 0.0749 | NA | NA | NA | NA |
| WCM0_PM1 | 14 | 89083185 | 93995606 | 0.1988 | −0.1004 | 0.9376 | 0.9279 | 1 | 1 |
| WCM0_PM1 | 14 | 93998888 | 95267286 | 0.4585 | 0.1699 | 1.153 | 1.097 | 1 | 1 |
| WCM0_PM1 | 14 | 95556812 | 95657985 | −0.602 | −0.9498 | NA | NA | NA | NA |
| WCM0_PM1 | 14 | 95660135 | 102252462 | 0.3844 | 0.093 | 1.0976 | 1.0356 | 1 | 1 |
| WCM0_PM1 | 14 | 102276301 | 104493246 | 0.0865 | −0.2179 | 0.8652 | 0.8544 | 1 | 1 |
| WCM0_PM1 | 14 | 104508456 | 107283105 | 0.2737 | −0.0222 | 0.9981 | 0.9714 | 1 | 1 |
| WCM0_PM1 | 15 | 20170117 | 40708277 | −0.0435 | −0.3545 | 1.0317 | 0.5325 | 1 | 1 |
| WCM0_PM1 | 15 | 40708546 | 42059256 | −0.4091 | −0.7424 | NA | NA | NA | NA |
| WCM0_PM1 | 15 | 42067548 | 43893123 | −0.1926 | −0.512 | 0.9264 | 0.4762 | 1 | 0 |
| WCM0_PM1 | 15 | 43893585 | 45206957 | −0.334 | −0.6622 | NA | NA | NA | NA |
| WCM0_PM1 | 15 | 45220859 | 50339649 | 0.0257 | −0.2816 | 1.0889 | 0.5564 | 1 | 1 |
| WCM0_PM1 | 15 | 50366356 | 64275947 | −0.1428 | −0.4593 | 0.968 | 0.4866 | 1 | 0 |
| WCM0_PM1 | 15 | 64332459 | 66850133 | −0.3277 | −0.6555 | NA | NA | NA | NA |
| WCM0_PM1 | 15 | 66850321 | 74574137 | −0.0734 | −0.3859 | 1.0066 | 0.524 | 1 | 1 |
| WCM0_PM1 | 15 | 74588261 | 76426561 | −0.2698 | −0.5939 | 0.8477 | 0.4773 | 1 | 0 |
| WCM0_PM1 | 15 | 76430111 | 90208910 | −0.0923 | −0.4059 | 0.9682 | 0.5413 | 1 | 1 |
| WCM0_PM1 | 15 | 90209135 | 91358441 | −0.3631 | −0.6933 | 0.8277 | 0.4092 | 1 | 0 |
| WCM0_PM1 | 15 | 91419099 | 93555670 | −0.1765 | −0.4949 | 0.9357 | 0.4835 | 1 | 0 |
| WCM0_PM1 | 15 | 93557907 | 102359175 | 0.0561 | −0.2498 | 1.0944 | 0.5877 | 1 | 1 |
| WCM0_PM1 | 16 | 79642 | 780924 | −0.7116 | −1.0685 | 0.9522 | 0.0014 | 1 | 0 |
| WCM0_PM1 | 16 | 781633 | 19800223 | −0.7938 | −1.1582 | 0.8986 | −0.0025 | 1 | 0 |
| WCM0_PM1 | 16 | 19838334 | 20800886 | −0.5086 | −0.8491 | NA | NA | NA | NA |
| WCM0_PM1 | 16 | 20801982 | 28145156 | −0.7785 | −1.1415 | 0.9082 | −0.0016 | 1 | 0 |
| WCM0_PM1 | 16 | 28146632 | 31049751 | −0.9077 | −1.2831 | 0.8304 | −0.0086 | 1 | 0 |
| WCM0_PM1 | 16 | 31050018 | 31155204 | −0.8114 | −1.1775 | NA | NA | NA | NA |
| WCM0_PM1 | 16 | 31157126 | 47409798 | −0.7085 | −1.0652 | 0.944 | 0.0118 | 1 | 0 |
| WCM0_PM1 | 16 | 47462746 | 56717096 | −0.5407 | −0.8837 | 1.0819 | 0.0021 | 1 | 0 |
| WCM0_PM1 | 16 | 56717863 | 66670382 | −0.6982 | −1.054 | 0.961 | 0.0023 | 1 | 0 |
| WCM0_PM1 | 16 | 66730217 | 70694103 | −0.8229 | −1.19 | 0.8842 | −0.0076 | 1 | 0 |
| WCM0_PM1 | 16 | 70697774 | 90162264 | −0.6529 | −1.0049 | 1.0005 | −0.0039 | 1 | 0 |
| WCM0_PM1 | 17 | 131595 | 531406 | 0.2289 | −0.0689 | NA | NA | NA | NA |
| WCM0_PM1 | 17 | 534752 | 5341450 | 0.0645 | −0.2409 | 0.8462 | 0.8462 | 1 | 1 |
| WCM0_PM1 | 17 | 5347688 | 7579892 | 0.2148 | −0.0836 | 0.9437 | 0.9437 | 1 | 1 |
| WCM0_PM1 | 17 | 7592111 | 7951897 | −0.7604 | −1.1217 | NA | NA | NA | NA |
| WCM0_PM1 | 17 | 7976288 | 10427223 | 0.1769 | −0.1232 | 0.9316 | 0.9047 | 1 | 1 |
| WCM0_PM1 | 17 | 10427923 | 15406427 | 0.3449 | 0.052 | 1.0453 | 1.028 | 1 | 1 |
| WCM0_PM1 | 17 | 15449153 | 28030047 | 0.1965 | −0.1027 | 0.9955 | 0.867 | 1 | 1 |
| WCM0_PM1 | 17 | 28120931 | 28773006 | 0.7289 | 0.4494 | NA | NA | NA | NA |
| WCM0_PM1 | 17 | 28776594 | 31065347 | −0.8137 | −1.18 | 0.8925 | −0.0098 | 1 | 0 |
| WCM0_PM1 | 17 | 31072245 | 35307467 | −0.5318 | −0.874 | 1.0871 | 0.0041 | 1 | 0 |
| WCM0_PM1 | 17 | 35307729 | 38636045 | −0.7797 | −1.1428 | 0.9143 | −0.0085 | 1 | 0 |
| WCM0_PM1 | 17 | 38636989 | 39728203 | −0.4739 | −0.8118 | 1.1296 | 0.0097 | 1 | 0 |
| WCM0_PM1 | 17 | 39728249 | 47921402 | −0.7834 | −1.1469 | 0.9103 | −0.0071 | 1 | 0 |
| WCM0_PM1 | 17 | 47925346 | 56604517 | −0.5811 | −0.9272 | 1.0514 | 3.00E−04 | 1 | 0 |
| WCM0_PM1 | 17 | 56606456 | 59853725 | −0.8205 | −1.1874 | 0.8886 | −0.0104 | 1 | 0 |
| WCM0_PM1 | 17 | 59857621 | 66526549 | 0.1099 | −0.1933 | 0.8883 | 0.8608 | 1 | 1 |
| WCM0_PM1 | 17 | 66533655 | 70645373 | 0.3339 | 0.0405 | 1.0724 | 0.9846 | 1 | 1 |
| WCM0_PM1 | 17 | 70732816 | 72877302 | 0.1367 | −0.1653 | 0.9776 | 0.8059 | 1 | 1 |
| WCM0_PM1 | 17 | 72878633 | 75303202 | 0.0197 | −0.288 | 0.8776 | 0.7605 | 1 | 1 |
| WCM0_PM1 | 17 | 75316383 | 81052243 | 0.163 | −0.1378 | 0.9821 | 0.8357 | 1 | 1 |
| WCM0_PM1 | 18 | 47646 | 14613572 | 0.8425 | 0.5664 | 1.939 | 1.0225 | 2 | 1 |
| WCM0_PM1 | 18 | 18539811 | 22030809 | −0.7553 | −1.1161 | 0.9221 | 6.00E−04 | 1 | 0 |
| WCM0_PM1 | 18 | 22040802 | 74980855 | −0.4754 | −0.8134 | 1.1248 | 0.0133 | 1 | 0 |
| WCM0_PM1 | 18 | 76753131 | 77287526 | 0.8945 | 0.6198 | NA | NA | NA | NA |
| WCM0_PM1 | 18 | 77440041 | 78005274 | −0.3486 | −0.6778 | NA | NA | NA | NA |
| WCM0_PM1 | 19 | 281360 | 6374940 | 0.0073 | −0.301 | 0.8194 | 0.804 | 1 | 1 |
| WCM0_PM1 | 19 | 6375471 | 15079170 | 0.3452 | 0.0522 | 1.2785 | 0.7953 | 1 | 1 |
| WCM0_PM1 | 19 | 15082537 | 16296543 | 0.5368 | 0.2511 | 1.466 | 0.9142 | 1 | 1 |
| WCM0_PM1 | 19 | 16301349 | 19466504 | 0.3453 | 0.0524 | 1.2732 | 0.8007 | 1 | 1 |
| WCM0_PM1 | 19 | 19478892 | 36128173 | 0.5081 | 0.2213 | 1.4411 | 0.8905 | 1 | 1 |
| WCM0_PM1 | 19 | 36128439 | 39688815 | 0.3431 | 0.0501 | 1.2679 | 0.8027 | 1 | 1 |
| WCM0_PM1 | 19 | 39689893 | 42597927 | 0.515 | 0.2284 | 1.4263 | 0.9168 | 1 | 1 |
| WCM0_PM1 | 19 | 42599631 | 42730600 | −0.0329 | −0.3433 | NA | NA | NA | NA |
| WCM0_PM1 | 19 | 42736292 | 42848999 | −4.2504 | −8.4086 | NA | NA | NA | NA |
| WCM0_PM1 | 19 | 42853720 | 44160711 | −0.062 | −0.374 | 1.5451 | −0.0018 | 2 | 0 |
| WCM0_PM1 | 19 | 44169650 | 45211467 | 0.5585 | 0.2735 | 1.4726 | 0.9449 | 1 | 1 |
| WCM0_PM1 | 19 | 45213783 | 48643195 | 0.3691 | 0.0771 | 1.2764 | 0.8334 | 1 | 1 |
| WCM0_PM1 | 19 | 48646883 | 51171634 | 0.057 | −0.2488 | 0.8531 | 0.8301 | 1 | 1 |
| WCM0_PM1 | 19 | 51171919 | 52543806 | 0.2801 | −0.0155 | 1.0079 | 0.9707 | 1 | 1 |
| WCM0_PM1 | 19 | 52544833 | 56569596 | 0.1139 | −0.1891 | 0.9534 | 0.8009 | 1 | 1 |
| WCM0_PM1 | 19 | 56572935 | 58721339 | 0.3735 | 0.0816 | 1.2078 | 0.9086 | 1 | 1 |
| WCM0_PM1 | 19 | 58723103 | 59082767 | 0.0723 | −0.2327 | 0.9662 | 0.7359 | 1 | 1 |
| WCM0_PM1 | 2 | 41613 | 9541373 | 0.3347 | 0.0413 | 1.0451 | 1.013 | 1 | 1 |
| WCM0_PM1 | 2 | 9543463 | 21266425 | −0.035 | −0.3456 | 0.9188 | 0.6552 | 1 | 1 |
| WCM0_PM1 | 2 | 23974938 | 29165168 | −0.4151 | −0.7488 | 0.697 | 0.4932 | 1 | 0 |
| WCM0_PM1 | 2 | 29169313 | 31805928 | −0.0654 | −0.3775 | 0.9101 | 0.6294 | 1 | 1 |
| WCM0_PM1 | 2 | 32093528 | 32897357 | −0.5985 | −0.946 | NA | NA | NA | NA |
| WCM0_PM1 | 2 | 32903985 | 39250247 | −0.3047 | −0.631 | 0.7578 | 0.5337 | 1 | 1 |
| WCM0_PM1 | 2 | 39251221 | 44934480 | −0.0646 | −0.3767 | 0.801 | 0.7393 | 1 | 1 |
| WCM0_PM1 | 2 | 44942462 | 60983595 | 0.1813 | −0.1186 | 1.0289 | 0.8132 | 1 | 1 |
| WCM0_PM1 | 2 | 60987435 | 62728379 | −0.1646 | −0.4823 | 0.7666 | 0.6651 | 1 | 1 |
| WCM0_PM1 | 2 | 62728589 | 68274471 | 0.1672 | −0.1334 | 0.9841 | 0.8392 | 1 | 1 |
| WCM0_PM1 | 2 | 68352543 | 69784085 | 0.4558 | 0.1672 | 1.1485 | 1.0972 | 1 | 1 |
| WCM0_PM1 | 2 | 69870158 | 72802770 | −0.5466 | −0.89 | 1.0791 | 2.00E−04 | 1 | 0 |
| WCM0_PM1 | 2 | 72945383 | 74867316 | −0.7502 | −1.1106 | 0.925 | 0.0012 | 1 | 0 |
| WCM0_PM1 | 2 | 74867419 | 85133176 | −0.5233 | −0.8649 | 1.0861 | 0.0121 | 1 | 0 |
| WCM0_PM1 | 2 | 85133552 | 86733121 | −0.9407 | −1.3195 | 0.8076 | −0.0063 | 1 | 0 |
| WCM0_PM1 | 2 | 86734630 | 91955271 | −0.689 | −1.0441 | 0.9389 | 0.031 | 1 | 0 |
| WCM0_PM1 | 2 | 91955350 | 109068837 | 0.3 | 0.0052 | 1.0214 | 0.9858 | 1 | 1 |
| WCM0_PM1 | 2 | 109085449 | 113959031 | 0.1251 | −0.1774 | 0.9149 | 0.8537 | 1 | 1 |
| WCM0_PM1 | 2 | 113976089 | 116101462 | −0.6291 | −0.9791 | NA | NA | NA | NA |
| WCM0_PM1 | 2 | 116257074 | 116539955 | 0.1741 | −0.1261 | NA | NA | NA | NA |
| WCM0_PM1 | 2 | 116548650 | 120690074 | 0.391 | 0.0998 | 1.0984 | 1.0449 | 1 | 1 |
| WCM0_PM1 | 2 | 120692493 | 149501213 | −0.6371 | −0.9878 | 1.006 | 0.0026 | 1 | 0 |
| WCM0_PM1 | 2 | 149511523 | 153192188 | 0.2848 | −0.0106 | 1.003 | 0.9823 | 1 | 1 |
| WCM0_PM1 | 2 | 153378459 | 170218914 | 0.4536 | 0.1648 | 1.1292 | 1.1129 | 1 | 1 |
| WCM0_PM1 | 2 | 170336047 | 175613600 | 0.2469 | −0.0501 | 0.9951 | 0.9366 | 1 | 1 |
| WCM0_PM1 | 2 | 175614684 | 179314930 | 0.4339 | 0.1444 | 1.1271 | 1.0835 | 1 | 1 |
| WCM0_PM1 | 2 | 179315781 | 182786948 | 0.5337 | 0.2479 | 1.2034 | 1.1715 | 1 | 1 |
| WCM0_PM1 | 2 | 182794384 | 197595567 | 0.4106 | 0.1202 | 1.1004 | 1.0734 | 1 | 1 |
| WCM0_PM1 | 2 | 197596876 | 201678011 | 0.3242 | 0.0304 | 1.0415 | 1.0011 | 1 | 1 |
| WCM0_PM1 | 2 | 201680222 | 204571465 | 0.1152 | −0.1878 | 0.9028 | 0.8531 | 1 | 1 |
| WCM0_PM1 | 2 | 204591428 | 219029182 | 0.3848 | 0.0935 | 1.0836 | 1.0503 | 1 | 1 |
| WCM0_PM1 | 2 | 219029682 | 220252494 | 0.2059 | −0.093 | 0.9484 | 0.9268 | 1 | 1 |
| WCM0_PM1 | 2 | 220283189 | 223918019 | 0.3676 | 0.0755 | 1.0746 | 1.0329 | 1 | 1 |
| WCM0_PM1 | 2 | 224462339 | 232458726 | 0.1181 | −0.1847 | 0.9001 | 0.8595 | 1 | 1 |
| WCM0_PM1 | 2 | 232573388 | 234344561 | −0.7354 | −1.0945 | 0.9472 | −0.0106 | 1 | 0 |
| WCM0_PM1 | 2 | 234345100 | 234621787 | 0.2214 | −0.0768 | 0.9799 | 0.9165 | 1 | 1 |
| WCM0_PM1 | 2 | 234622061 | 234656640 | 0.4466 | 0.1576 | NA | NA | NA | NA |
| WCM0_PM1 | 2 | 234669127 | 242003080 | 0.4242 | 0.1343 | 1.1056 | 1.0896 | 1 | 1 |
| WCM0_PM1 | 2 | 242003502 | 242815462 | 0.2467 | −0.0504 | 0.9868 | 0.9445 | 1 | 1 |
| WCM0_PM1 | 20 | 68342 | 2398091 | 0.455 | 0.1663 | 1.128 | 1.1164 | 1 | 1 |
| WCM0_PM1 | 20 | 2398184 | 6011999 | 0.2195 | −0.0787 | 0.9617 | 0.9321 | 1 | 1 |
| WCM0_PM1 | 20 | 6012706 | 26188920 | 0.4887 | 0.2013 | 1.1664 | 1.133 | 1 | 1 |
| WCM0_PM1 | 20 | 29845457 | 36012697 | 0.1078 | −0.1955 | 0.8769 | 0.8696 | 1 | 1 |
| WCM0_PM1 | 20 | 36012825 | 45808514 | 0.3345 | 0.0412 | 1.0375 | 1.0204 | 1 | 1 |
| WCM0_PM1 | 20 | 45809420 | 50803444 | 0.1156 | −0.1874 | 0.8782 | 0.8782 | 1 | 1 |
| WCM0_PM1 | 20 | 50803631 | 55748259 | 0.3117 | 0.0174 | NA | NA | NA | NA |
| WCM0_PM1 | 20 | 55750037 | 60573214 | 0.4966 | 0.2094 | 1.167 | 1.1455 | 1 | 1 |
| WCM0_PM1 | 20 | 60574216 | 62076164 | 0.312 | 0.0177 | 1.025 | 0.9997 | 1 | 1 |
| WCM0_PM1 | 20 | 62076743 | 62642800 | 0.158 | −0.143 | 0.9094 | 0.9018 | 1 | 1 |
| WCM0_PM1 | 20 | 62648208 | 62904890 | 0.4196 | 0.1296 | NA | NA | NA | NA |
| WCM0_PM1 | 21 | 10862618 | 14917181 | −0.0493 | −0.3606 | 0.8962 | 0.6615 | 1 | 1 |
| WCM0_PM1 | 21 | 15399864 | 32513395 | 0.4737 | 0.1856 | 1.1385 | 1.1361 | 1 | 1 |
| WCM0_PM1 | 21 | 32513843 | 36164292 | 0.2352 | −0.0624 | 0.9807 | 0.9347 | 1 | 1 |
| WCM0_PM1 | 21 | 36164704 | 37507720 | −0.6577 | −1.0101 | NA | NA | NA | NA |
| WCM0_PM1 | 21 | 37510210 | 38538354 | 0.1866 | −0.1131 | 0.9632 | 0.8859 | 1 | 1 |
| WCM0_PM1 | 21 | 38538782 | 44119121 | 0.4727 | 0.1847 | 1.146 | 1.1271 | 1 | 1 |
| WCM0_PM1 | 21 | 44152088 | 45846593 | 0.2926 | −0.0025 | 1.044 | 0.9525 | 1 | 1 |
| WCM0_PM1 | 21 | 45846961 | 48084270 | 0.498 | 0.2109 | 1.1646 | 1.1502 | 1 | 1 |
| WCM0_PM1 | 22 | 16255826 | 29095876 | −0.6626 | −1.0153 | 0.9882 | 0.0012 | 1 | 0 |
| WCM0_PM1 | 22 | 29099475 | 32546363 | −0.7859 | −1.1495 | 0.9048 | −0.0033 | 1 | 0 |
| WCM0_PM1 | 22 | 32546978 | 37904675 | −0.6525 | −1.0044 | 0.9933 | 0.0037 | 1 | 0 |
| WCM0_PM1 | 22 | 37906205 | 43435812 | −0.8534 | −1.2235 | 0.87 | −0.0135 | 1 | 0 |
| WCM0_PM1 | 22 | 43435962 | 51216446 | −0.6654 | −1.0184 | 0.9862 | 0.0012 | 1 | 0 |
| WCM0_PM1 | 3 | 361508 | 9290644 | −0.391 | −0.7231 | 1.2021 | 0.0095 | 1 | 0 |
| WCM0_PM1 | 3 | 9406871 | 10261375 | −0.7796 | −1.1427 | 0.9252 | −0.0194 | 1 | 0 |
| WCM0_PM1 | 3 | 10264480 | 32169695 | −0.5063 | −0.8466 | 1.106 | 0.0062 | 1 | 0 |
| WCM0_PM1 | 3 | 32180053 | 33759443 | −0.7379 | −1.0972 | NA | NA | NA | NA |
| WCM0_PM1 | 3 | 33840335 | 47044753 | −0.4706 | −0.8084 | 1.1329 | 0.0092 | 1 | 0 |
| WCM0_PM1 | 3 | 47045450 | 50405087 | −0.8101 | −1.1761 | 0.8977 | −0.0126 | 1 | 0 |
| WCM0_PM1 | 3 | 50405270 | 53535717 | −0.6142 | −0.963 | 1.0332 | −0.0073 | 1 | 0 |
| WCM0_PM1 | 3 | 53684957 | 56468391 | −0.381 | −0.7124 | 1.2127 | 0.0079 | 1 | 0 |
| WCM0_PM1 | 3 | 56468819 | 58620019 | −0.7522 | −1.1128 | 0.9216 | 0.0032 | 1 | 0 |
| WCM0_PM1 | 3 | 58622041 | 69058783 | −0.3751 | −0.706 | 1.2042 | 0.0218 | 1 | 0 |
| WCM0_PM1 | 3 | 69072402 | 73657790 | −0.5822 | −0.9284 | 1.033 | 0.0179 | 1 | 0 |
| WCM0_PM1 | 3 | 73673381 | 77526717 | 0.8436 | 0.5675 | NA | NA | NA | NA |
| WCM0_PM1 | 3 | 77530270 | 93768205 | 0.2263 | −0.0716 | NA | NA | NA | NA |
| WCM0_PM1 | 3 | 93769707 | 122103160 | −0.5075 | −0.8479 | 1.0991 | 0.0121 | 1 | 0 |
| WCM0_PM1 | 3 | 122121627 | 122598192 | 0.275 | −0.0208 | 1.0083 | 0.9631 | 1 | 1 |
| WCM0_PM1 | 3 | 122629084 | 124377324 | 0.506 | 0.2192 | 1.2171 | 1.111 | 1 | 1 |
| WCM0_PM1 | 3 | 124378199 | 125702134 | 0.2866 | −0.0088 | 1.0374 | 0.9504 | 1 | 1 |
| WCM0_PM1 | 3 | 125725378 | 128205826 | 0.5577 | 0.2726 | 1.2642 | 1.1518 | 1 | 1 |
| WCM0_PM1 | 3 | 128292324 | 129280522 | 0.1919 | −0.1076 | 0.9281 | 0.9281 | 1 | 1 |
| WCM0_PM1 | 3 | 129280739 | 134968204 | 0.4445 | 0.1554 | 1.1276 | 1.0999 | 1 | 1 |
| WCM0_PM1 | 3 | 134977937 | 138478136 | 0.1868 | −0.1128 | NA | NA | NA | NA |
| WCM0_PM1 | 3 | 138664453 | 141164727 | 0.5277 | 0.2416 | NA | NA | NA | NA |
| WCM0_PM1 | 3 | 141206018 | 142607722 | 0.1675 | −0.1331 | 0.9424 | 0.8814 | 1 | 1 |
| WCM0_PM1 | 3 | 142681100 | 169492339 | 0.431 | 0.1414 | 1.1076 | 1.0984 | 1 | 1 |
| WCM0_PM1 | 3 | 169496714 | 170185108 | 0.1134 | −0.1896 | 0.8998 | 0.8539 | 1 | 1 |
| WCM0_PM1 | 3 | 170198275 | 182563242 | 0.313 | 0.0188 | 1.0363 | 0.9899 | 1 | 1 |
| WCM0_PM1 | 3 | 182566325 | 186760943 | 0.1432 | −0.1585 | 0.9063 | 0.8856 | 1 | 1 |
| WCM0_PM1 | 3 | 186761066 | 193364861 | 0.4833 | 0.1956 | 1.1701 | 1.1203 | 1 | 1 |
| WCM0_PM1 | 3 | 193365903 | 197847274 | 0.1372 | −0.1648 | 0.9005 | 0.8837 | 1 | 1 |
| WCM0_PM1 | 4 | 53435 | 3237933 | 0.19 | −0.1096 | 0.9385 | 0.9152 | 1 | 1 |
| WCM0_PM1 | 4 | 3240113 | 25032243 | 0.4002 | 0.1095 | 1.0788 | 1.0788 | 1 | 1 |
| WCM0_PM1 | 4 | 25125641 | 38995588 | 0.2371 | −0.0604 | 0.961 | 0.957 | 1 | 1 |
| WCM0_PM1 | 4 | 39000305 | 41270001 | −0.0604 | −0.3723 | 0.7799 | 0.7652 | 1 | 1 |
| WCM0_PM1 | 4 | 41362912 | 44631587 | −0.5803 | −0.9264 | 1.0503 | 0.0021 | 1 | 0 |
| WCM0_PM1 | 4 | 44637961 | 56304722 | 0.3709 | 0.079 | 1.087 | 1.0255 | 1 | 1 |
| WCM0_PM1 | 4 | 56308640 | 57906986 | 0.1793 | −0.1208 | 0.9312 | 0.9082 | 1 | 1 |
| WCM0_PM1 | 4 | 57976194 | 71465315 | 0.4365 | 0.1471 | 1.1418 | 1.0729 | 1 | 1 |
| WCM0_PM1 | 4 | 71467297 | 73615956 | −0.0935 | −0.4072 | NA | NA | NA | NA |
| WCM0_PM1 | 4 | 73923944 | 75971460 | 0.4857 | 0.1981 | NA | NA | NA | NA |
| WCM0_PM1 | 4 | 76407857 | 77637502 | 0.186 | −0.1137 | 0.9484 | 0.9001 | 1 | 1 |
| WCM0_PM1 | 4 | 77651910 | 83276513 | 0.4687 | 0.1805 | 1.1333 | 1.1333 | 1 | 1 |
| WCM0_PM1 | 4 | 83277845 | 84390210 | 0.1327 | −0.1694 | 0.9292 | 0.8492 | 1 | 1 |
| WCM0_PM1 | 4 | 84391521 | 103912808 | 0.3901 | 0.0989 | 1.081 | 1.061 | 1 | 1 |
| WCM0_PM1 | 4 | 103947532 | 106158545 | 0.0074 | −0.3009 | NA | NA | NA | NA |
| WCM0_PM1 | 4 | 106290955 | 106604173 | −1.1952 | −1.6032 | NA | NA | NA | NA |
| WCM0_PM1 | 4 | 106604462 | 177073054 | 0.0382 | −0.2685 | 1.0647 | 0.5956 | 1 | 1 |
| WCM0_PM1 | 4 | 177077313 | 178911620 | −1.251 | −1.6663 | NA | NA | NA | NA |
| WCM0_PM1 | 4 | 183090528 | 190947555 | −0.0435 | −0.3544 | 0.9873 | 0.577 | 1 | 1 |
| WCM0_PM1 | 5 | 140480 | 1489877 | 0.388 | 0.0968 | 1.0883 | 1.0505 | 1 | 1 |
| WCM0_PM1 | 5 | 1494933 | 9630007 | 0.6138 | 0.3307 | 1.2784 | 1.2368 | 1 | 1 |
| WCM0_PM1 | 5 | 10227737 | 54529379 | 0.3988 | 0.108 | 1.0889 | 1.0665 | 1 | 1 |
| WCM0_PM1 | 5 | 54552305 | 57755768 | 0.14 | −0.1618 | NA | NA | NA | NA |
| WCM0_PM1 | 5 | 57790431 | 59901600 | 0.0145 | −0.2935 | NA | NA | NA | NA |
| WCM0_PM1 | 5 | 59934574 | 81572366 | −0.1184 | −0.4334 | 0.9845 | 0.4965 | 1 | 0 |
| WCM0_PM1 | 5 | 81573511 | 122950100 | 0.0576 | −0.2481 | 1.0717 | 0.6122 | 1 | 1 |
| WCM0_PM1 | 5 | 123966399 | 129072749 | −1.2818 | −1.7012 | 0.6127 | 0.0023 | 1 | 0 |
| WCM0_PM1 | 5 | 129240561 | 134345063 | −0.661 | −1.0136 | 0.9952 | −0.0046 | 1 | 0 |
| WCM0_PM1 | 5 | 134346022 | 136957742 | −0.255 | −0.5782 | 1.3265 | 0.0131 | 1 | 0 |
| WCM0_PM1 | 5 | 136961483 | 139006407 | 0.0861 | −0.2183 | 0.8839 | 0.8352 | 1 | 1 |
| WCM0_PM1 | 5 | 139060141 | 154065496 | 0.401 | 0.1103 | 1.0794 | 1.0794 | 1 | 1 |
| WCM0_PM1 | 5 | 154092593 | 159855634 | 0.2516 | −0.0452 | 0.9914 | 0.9469 | 1 | 1 |
| WCM0_PM1 | 5 | 159912418 | 170319530 | 0.4571 | 0.1685 | 1.146 | 1.1017 | 1 | 1 |
| WCM0_PM1 | 5 | 170323125 | 180166834 | 0.1899 | −0.1096 | 0.9317 | 0.922 | 1 | 1 |
| WCM0_PM1 | 5 | 180166891 | 180687814 | 0.4109 | 0.1205 | NA | NA | NA | NA |
| WCM0_PM1 | 6 | 292608 | 16328482 | 0.3718 | 0.08 | 1.0658 | 1.0482 | 1 | 1 |
| WCM0_PM1 | 6 | 17283085 | 18256677 | 0.0798 | −0.2249 | NA | NA | NA | NA |
| WCM0_PM1 | 6 | 18258297 | 30314271 | 0.3781 | 0.0865 | 1.0673 | 1.0563 | 1 | 1 |
| WCM0_PM1 | 6 | 30314598 | 38562047 | 0.225 | −0.073 | 0.9547 | 0.9467 | 1 | 1 |
| WCM0_PM1 | 6 | 38565844 | 41652604 | 0.471 | 0.1828 | 1.1504 | 1.1198 | 1 | 1 |
| WCM0_PM1 | 6 | 41652852 | 43648896 | 0.1122 | −0.191 | 0.8824 | 0.8696 | 1 | 1 |
| WCM0_PM1 | 6 | 43655455 | 73332336 | 0.4638 | 0.1754 | 1.1398 | 1.1187 | 1 | 1 |
| WCM0_PM1 | 6 | 73713724 | 74530283 | 0.1576 | −0.1435 | NA | NA | NA | NA |
| WCM0_PM1 | 6 | 74533212 | 86333835 | 0.415 | 0.1247 | 1.1118 | 1.0688 | 1 | 1 |
| WCM0_PM1 | 6 | 86346748 | 90605797 | 0.2209 | −0.0772 | NA | NA | NA | NA |
| WCM0_PM1 | 6 | 90642117 | 110763540 | 0.4092 | 0.1188 | 1.0914 | 1.0802 | 1 | 1 |
| WCM0_PM1 | 6 | 110763745 | 112029172 | 0.1821 | −0.1179 | 1.0381 | 0.805 | 1 | 1 |
| WCM0_PM1 | 6 | 112035620 | 131211637 | 0.5123 | 0.2257 | 1.1754 | 1.1633 | 1 | 1 |
| WCM0_PM1 | 6 | 131215462 | 139694266 | 0.365 | 0.0729 | 1.0595 | 1.0441 | 1 | 1 |
| WCM0_PM1 | 6 | 139694699 | 149394956 | 0.5092 | 0.2225 | 1.1885 | 1.145 | 1 | 1 |
| WCM0_PM1 | 6 | 149395274 | 151789542 | 0.1674 | −0.1332 | 0.9204 | 0.9032 | 1 | 1 |
| WCM0_PM1 | 6 | 151789888 | 158485969 | 0.382 | 0.0906 | 1.0913 | 1.0382 | 1 | 1 |
| WCM0_PM1 | 6 | 158487658 | 159650900 | 0.2458 | −0.0513 | 0.9922 | 0.9379 | 1 | 1 |
| WCM0_PM1 | 6 | 159653100 | 170893364 | 0.5075 | 0.2207 | 1.1701 | 1.1605 | 1 | 1 |
| WCM0_PM1 | 7 | 193625 | 4876252 | 0.184 | −0.1158 | 0.9229 | 0.9229 | 1 | 1 |
| WCM0_PM1 | 7 | 4899606 | 7278109 | −0.059 | −0.3708 | 0.7799 | 0.7668 | 1 | 1 |
| WCM0_PM1 | 7 | 7278227 | 22862367 | 0.4976 | 0.2105 | 1.1798 | 1.1343 | 1 | 1 |
| WCM0_PM1 | 7 | 22896231 | 26903972 | 0.2648 | −0.0315 | 0.9978 | 0.959 | 1 | 1 |
| WCM0_PM1 | 7 | 27134150 | 27196110 | 0.6097 | 0.3264 | NA | NA | NA | NA |
| WCM0_PM1 | 7 | 27203255 | 27689290 | −0.37 | −0.7006 | NA | NA | NA | NA |
| WCM0_PM1 | 7 | 27702390 | 29160538 | 0.4743 | 0.1863 | NA | NA | NA | NA |
| WCM0_PM1 | 7 | 29394233 | 38398397 | −0.4143 | −0.748 | 1.1797 | 0.0112 | 1 | 0 |
| WCM0_PM1 | 7 | 38402612 | 43485137 | 0.5094 | 0.2226 | NA | NA | NA | NA |
| WCM0_PM1 | 7 | 43490497 | 45197433 | 0.274 | −0.0219 | 0.9952 | 0.9747 | 1 | 1 |
| WCM0_PM1 | 7 | 45217038 | 55565379 | 0.5408 | 0.2552 | 1.212 | 1.1749 | 1 | 1 |
| WCM0_PM1 | 7 | 55588784 | 77583111 | 0.0449 | −0.2615 | 0.8439 | 0.8246 | 1 | 1 |
| WCM0_PM1 | 7 | 77584210 | 90896080 | 0.4815 | 0.1938 | 1.1687 | 1.1189 | 1 | 1 |
| WCM0_PM1 | 7 | 91503070 | 92166221 | 0.2676 | −0.0286 | NA | NA | NA | NA |
| WCM0_PM1 | 7 | 92195327 | 97364194 | 0.5019 | 0.2149 | 1.1631 | 1.1583 | 1 | 1 |
| WCM0_PM1 | 7 | 97365650 | 102608570 | 0.0075 | −0.3009 | 0.9227 | 0.7008 | 1 | 1 |
| WCM0_PM1 | 7 | 102614133 | 108213585 | 0.3001 | 0.0053 | 1.0068 | 1.0005 | 1 | 1 |
| WCM0_PM1 | 7 | 108524245 | 127892028 | 0.4859 | 0.1984 | 1.1605 | 1.1343 | 1 | 1 |
| WCM0_PM1 | 7 | 127894487 | 131190676 | 0.1608 | −0.14 | 0.9236 | 0.8914 | 1 | 1 |
| WCM0_PM1 | 7 | 131191055 | 137612956 | 0.4359 | 0.1465 | 1.1161 | 1.0977 | 1 | 1 |
| WCM0_PM1 | 7 | 137686457 | 140710368 | 0.1109 | −0.1923 | 0.8789 | 0.8715 | 1 | 1 |
| WCM0_PM1 | 7 | 141255308 | 144098992 | 0.5513 | 0.266 | 1.3257 | 1.0793 | 1 | 1 |
| WCM0_PM1 | 7 | 144101768 | 149152541 | 0.2014 | −0.0976 | 0.9424 | 0.9268 | 1 | 1 |
| WCM0_PM1 | 7 | 149152770 | 150774772 | 0.429 | 0.1393 | 1.1128 | 1.0899 | 1 | 1 |
| WCM0_PM1 | 7 | 150775162 | 158727122 | 0.3006 | 0.0058 | 1.0145 | 0.9936 | 1 | 1 |
| WCM0_PM1 | 7 | 158734610 | 158937416 | 0.6233 | 0.3405 | NA | NA | NA | NA |
| WCM0_PM1 | 8 | 192883 | 2092894 | −3.8743 | −5.9454 | NA | NA | NA | NA |
| WCM0_PM1 | 8 | 2796215 | 36703373 | −0.4713 | −0.8091 | 1.1403 | 0.0012 | 1 | 0 |
| WCM0_PM1 | 8 | 36721957 | 38688621 | 0.1375 | −0.1644 | 0.9091 | 0.8755 | 1 | 1 |
| WCM0_PM1 | 8 | 38693742 | 41791592 | 0.3814 | 0.0899 | 1.0787 | 1.0499 | 1 | 1 |
| WCM0_PM1 | 8 | 41792208 | 48889344 | 0.1528 | −0.1484 | NA | NA | NA | NA |
| WCM0_PM1 | 8 | 48921027 | 67372603 | 0.4862 | 0.1986 | 1.1607 | 1.1345 | 1 | 1 |
| WCM0_PM1 | 8 | 67372731 | 68255528 | 0.1361 | −0.166 | NA | NA | NA | NA |
| WCM0_PM1 | 8 | 68334717 | 95264305 | 0.4649 | 0.1766 | 1.1419 | 1.1185 | 1 | 1 |
| WCM0_PM1 | 8 | 95265306 | 105601037 | 0.3253 | 0.0315 | 1.0285 | 1.0157 | 1 | 1 |
| WCM0_PM1 | 8 | 106431004 | 120435135 | −0.3342 | −0.6625 | 1.2527 | 0.0109 | 1 | 0 |
| WCM0_PM1 | 8 | 120435395 | 131249188 | −0.5304 | −0.8726 | 1.0905 | 0.0018 | 1 | 0 |
| WCM0_PM1 | 8 | 131370344 | 140744432 | −0.2546 | −0.5777 | 1.3234 | 0.0167 | 1 | 0 |
| WCM0_PM1 | 8 | 140898197 | 144240384 | −0.4771 | −0.8153 | 1.1356 | 0.001 | 1 | 0 |
| WCM0_PM1 | 8 | 144241646 | 145999910 | −0.6384 | −0.9892 | 1.0094 | −0.0019 | 1 | 0 |
| WCM0_PM1 | 8 | 146002848 | 146279508 | −0.4378 | −0.7732 | NA | NA | NA | NA |
| WCM0_PM1 | 9 | 117666 | 3395457 | −0.7173 | −1.0748 | 0.9436 | 0.0058 | 1 | 0 |
| WCM0_PM1 | 9 | 3828235 | 19032907 | 0.2456 | −0.0515 | 1.0616 | 0.8683 | 1 | 1 |
| WCM0_PM1 | 9 | 19049648 | 19597249 | −0.6649 | −1.0179 | NA | NA | NA | NA |
| WCM0_PM1 | 9 | 19619597 | 33026828 | −0.4495 | −0.7857 | 1.139 | 0.0211 | 1 | 0 |
| WCM0_PM1 | 9 | 33029893 | 34506885 | −0.7373 | −1.0966 | NA | NA | NA | NA |
| WCM0_PM1 | 9 | 34512211 | 35842694 | −0.5139 | −0.8549 | 1.11 | −0.0042 | 1 | 0 |
| WCM0_PM1 | 9 | 35845979 | 40772470 | −0.6635 | −1.0164 | 0.9735 | 0.0152 | 1 | 0 |
| WCM0_PM1 | 9 | 40773337 | 99797060 | 0.1276 | −0.1748 | 0.9638 | 0.808 | 1 | 1 |
| WCM0_PM1 | 9 | 99797856 | 114090601 | 0.321 | 0.0271 | 1.0264 | 1.0115 | 1 | 1 |
| WCM0_PM1 | 9 | 114124372 | 116191674 | 0.0702 | −0.235 | 0.8702 | 0.8292 | 1 | 1 |
| WCM0_PM1 | 9 | 116224126 | 116303619 | 0.2982 | 0.0033 | NA | NA | NA | NA |
| WCM0_PM1 | 9 | 116345812 | 123170668 | 0.468 | 0.1798 | 1.1327 | 1.1327 | 1 | 1 |
| WCM0_PM1 | 9 | 123170795 | 127265672 | 0.2489 | −0.0481 | 0.9672 | 0.9672 | 1 | 1 |
| WCM0_PM1 | 9 | 127285000 | 130476368 | 0.0975 | −0.2063 | 0.8785 | 0.8549 | 1 | 1 |
| WCM0_PM1 | 9 | 130476641 | 134322439 | −0.0381 | −0.3488 | 0.7944 | 0.7762 | 1 | 1 |
| WCM0_PM1 | 9 | 134323136 | 139402865 | 0.2149 | −0.0835 | 0.9526 | 0.9349 | 1 | 1 |
| WCM0_PM1 | 9 | 139403379 | 140676804 | 0.0747 | −0.2303 | 0.8596 | 0.8453 | 1 | 1 |
| WCM0_PM1 | 9 | 140685306 | 141071438 | 0.4547 | 0.166 | NA | NA | NA | NA |
| WCM0_PM1 | X | 2700159 | 3592610 | 0.1448 | −0.1568 | NA | NA | NA | NA |
| WCM0_PM1 | X | 3631202 | 18183347 | 0.323 | 0.0292 | NA | NA | NA | NA |
| WCM0_PM1 | X | 18189182 | 24948585 | 0.1171 | −0.1858 | NA | NA | NA | NA |
| WCM0_PM1 | X | 25013914 | 38664744 | 0.3986 | 0.1077 | NA | NA | NA | NA |
| WCM0_PM1 | X | 39911398 | 54569441 | 0.1373 | −0.1647 | NA | NA | NA | NA |
| WCM0_PM1 | X | 54569744 | 69353845 | 0.4412 | 0.1519 | NA | NA | NA | NA |
| WCM0_PM1 | X | 69354439 | 69849453 | 0.2956 | 6.00E−04 | NA | NA | NA | NA |
| WCM0_PM1 | X | 69871362 | 70832741 | 0.0326 | −0.2745 | NA | NA | NA | NA |
| WCM0_PM1 | X | 70836210 | 100662774 | 0.3119 | 0.0176 | NA | NA | NA | NA |
| WCM0_PM1 | X | 100808315 | 117053697 | 0.5443 | 0.2588 | NA | NA | NA | NA |
| WCM0_PM1 | X | 117054343 | 123234407 | 0.078 | −0.2268 | NA | NA | NA | NA |
| WCM0_PM1 | X | 123480499 | 140967161 | 0.3668 | 0.0747 | NA | NA | NA | NA |
| WCM0_PM1 | X | 140969288 | 152913479 | 0.6229 | 0.3401 | NA | NA | NA | NA |
| WCM0_PM1 | X | 152913789 | 154509348 | 0.2929 | −0.0022 | NA | NA | NA | NA |
| WCM0_PM2 | 1 | 809237 | 1019882 | 0.4381 | 0.4965 | 1.4423 | 1.3793 | 1 | 1 |
| WCM0_PM2 | 1 | 1021415 | 2560735 | 0.0847 | 0.1373 | 1.1054 | 1.0942 | 1 | 1 |
| WCM0_PM2 | 1 | 2938344 | 6253026 | 0.47 | 0.5289 | 1.4648 | 1.4209 | 1 | 1 |
| WCM0_PM2 | 1 | 6257789 | 11986055 | 0.0964 | 0.1492 | 1.1169 | 1.1011 | 1 | 1 |
| WCM0_PM2 | 1 | 11994929 | 15546134 | −0.75 | −0.7185 | 1.2226 | −0.0072 | 1 | 0 |
| WCM0_PM2 | 1 | 15736699 | 16782267 | −0.9022 | −0.8761 | 1.0982 | −0.0085 | 1 | 0 |
| WCM0_PM2 | 1 | 16785343 | 19609254 | −0.6473 | −0.6125 | 1.3083 | −2.00E−04 | 1 | 0 |
| WCM0_PM2 | 1 | 19611241 | 19652872 | 0.3969 | 0.4548 | NA | NA | NA | NA |
| WCM0_PM2 | 1 | 19653099 | 23346302 | 0.2334 | 0.2886 | 1.2327 | 1.2103 | 1 | 1 |
| WCM0_PM2 | 1 | 23357092 | 29487055 | 0.049 | 0.1009 | 1.0768 | 1.0681 | 1 | 1 |
| WCM0_PM2 | 1 | 29508220 | 32280034 | 0.3287 | 0.3854 | 1.3275 | 1.285 | 1 | 1 |
| WCM0_PM2 | 1 | 32280324 | 33480094 | 0.0298 | 0.0814 | 1.071 | 1.0451 | 1 | 1 |
| WCM0_PM2 | 1 | 33487017 | 33613286 | 0.3227 | 0.3794 | NA | NA | NA | NA |
| WCM0_PM2 | 1 | 33623854 | 35370751 | 0.6404 | 0.7015 | NA | NA | NA | NA |
| WCM0_PM2 | 1 | 35370988 | 41013119 | 0.1609 | 0.2149 | 1.1677 | 1.1536 | 1 | 1 |
| WCM0_PM2 | 1 | 41092296 | 44750540 | 0.4095 | 0.4675 | 1.4093 | 1.356 | 1 | 1 |
| WCM0_PM2 | 1 | 44774068 | 46827425 | 0.1694 | 0.2235 | NA | NA | NA | NA |
| WCM0_PM2 | 1 | 46827456 | 51875170 | 0.4529 | 0.5115 | 1.4588 | 1.3923 | 1 | 1 |
| WCM0_PM2 | 1 | 51887488 | 53527948 | 0.0887 | 0.1414 | NA | NA | NA | NA |
| WCM0_PM2 | 1 | 53535684 | 61824888 | 0.4866 | 0.5457 | 1.4597 | 1.4597 | 1 | 1 |
| WCM0_PM2 | 1 | 61849014 | 63294767 | 0.1837 | 0.2381 | 1.1998 | 1.159 | 1 | 1 |
| WCM0_PM2 | 1 | 63299775 | 91182628 | 0.5166 | 0.5762 | 1.4909 | 1.4909 | 1 | 1 |
| WCM0_PM2 | 1 | 91382469 | 94027827 | −0.7115 | −0.6788 | 1.255 | −0.0056 | 1 | 0 |
| WCM0_PM2 | 1 | 94032886 | 109159008 | −0.439 | −0.3981 | 1.5147 | 0.003 | 2 | 0 |
| WCM0_PM2 | 1 | 109167265 | 110019441 | −0.7296 | −0.6975 | 1.2391 | −0.0058 | 1 | 0 |
| WCM0_PM2 | 1 | 110020456 | 112991789 | −0.3925 | −0.3504 | 1.5634 | 0.0053 | 2 | 0 |
| WCM0_PM2 | 1 | 112997121 | 115402411 | −0.6649 | −0.6306 | 1.2975 | −0.0057 | 1 | 0 |
| WCM0_PM2 | 1 | 115403368 | 119469188 | −0.3717 | −0.3291 | 1.5962 | −0.0041 | 2 | 0 |
| WCM0_PM2 | 1 | 119474318 | 150065790 | −0.1948 | −0.1479 | 1.232 | 0.573 | 1 | 1 |
| WCM0_PM2 | 1 | 150082704 | 151239077 | −0.4852 | −0.4456 | NA | NA | NA | NA |
| WCM0_PM2 | 1 | 151258751 | 151752629 | −0.2369 | −0.191 | NA | NA | NA | NA |
| WCM0_PM2 | 1 | 151753952 | 153520144 | 0.2668 | 0.3226 | 1.7285 | 0.7727 | 2 | 1 |
| WCM0_PM2 | 1 | 153520352 | 155231927 | −0.2819 | −0.237 | 1.13 | 0.567 | 1 | 1 |
| WCM0_PM2 | 1 | 155233071 | 156337543 | −0.4408 | −0.3999 | 0.9922 | 0.5236 | 1 | 1 |
| WCM0_PM2 | 1 | 156339145 | 156764618 | −0.1454 | −0.0974 | NA | NA | NA | NA |
| WCM0_PM2 | 1 | 156767035 | 160106856 | 0.1965 | 0.2512 | 1.5667 | 0.8137 | 2 | 1 |
| WCM0_PM2 | 1 | 160109420 | 160793560 | 0.0495 | 0.1015 | NA | NA | NA | NA |
| WCM0_PM2 | 1 | 160793962 | 161487982 | −0.2583 | −0.2128 | NA | NA | NA | NA |
| WCM0_PM2 | 1 | 161494726 | 173495771 | 0.1055 | 0.1586 | 1.4731 | 0.7593 | 1 | 1 |
| WCM0_PM2 | 1 | 173499026 | 175037036 | −0.1316 | −0.0833 | NA | NA | NA | NA |
| WCM0_PM2 | 1 | 175046621 | 178745891 | 0.2552 | 0.3109 | 1.6326 | 0.8483 | 2 | 1 |
| WCM0_PM2 | 1 | 178753549 | 180067988 | −0.1776 | −0.1303 | 1.1686 | 0.6586 | 1 | 1 |
| WCM0_PM2 | 1 | 180080115 | 182353636 | 0.1853 | 0.2397 | 1.5856 | 0.776 | 2 | 1 |
| WCM0_PM2 | 1 | 182354850 | 185278604 | 0.0023 | 0.0533 | 1.3466 | 0.7287 | 1 | 1 |
| WCM0_PM2 | 1 | 185703989 | 198828264 | 0.167 | 0.2212 | 1.563 | 0.7683 | 2 | 1 |
| WCM0_PM2 | 1 | 199997047 | 205890934 | −0.0831 | −0.0338 | 1.2511 | 0.7026 | 1 | 1 |
| WCM0_PM2 | 1 | 205892250 | 206822344 | 0.099 | 0.1519 | NA | NA | NA | NA |
| WCM0_PM2 | 1 | 206902448 | 210111622 | 0.2408 | 0.2961 | NA | NA | NA | NA |
| WCM0_PM2 | 1 | 210126087 | 213436143 | −0.1164 | −0.0678 | 1.2689 | 0.6393 | 1 | 1 |
| WCM0_PM2 | 1 | 213445947 | 223934893 | 0.1067 | 0.1598 | 1.4675 | 0.7668 | 1 | 1 |
| WCM0_PM2 | 1 | 223936750 | 226567827 | −0.1439 | −0.0959 | 1.2225 | 0.6488 | 1 | 1 |
| WCM0_PM2 | 1 | 226568905 | 233225848 | 0.3017 | 0.358 | 1.3012 | 1.2621 | 1 | 1 |
| WCM0_PM2 | 1 | 233231500 | 233394479 | −0.7106 | −0.6778 | NA | NA | NA | NA |
| WCM0_PM2 | 1 | 233394735 | 234745243 | 0.321 | 0.3777 | 1.3402 | 1.2584 | 1 | 1 |
| WCM0_PM2 | 1 | 235277167 | 236881136 | −0.06 | −0.0102 | 1.0041 | 0.9818 | 1 | 1 |
| WCM0_PM2 | 1 | 236882331 | 247902251 | 0.2179 | 0.2729 | 1.2844 | 1.1321 | 1 | 1 |
| WCM0_PM2 | 1 | 247902669 | 249212574 | 0.5629 | 0.6231 | 1.7742 | 1.3061 | 2 | 1 |
| WCM0_PM2 | 10 | 226048 | 5454644 | −0.3654 | −0.3226 | 1.5926 | 0.0067 | 2 | 0 |
| WCM0_PM2 | 10 | 5468695 | 11574178 | −0.6543 | −0.6197 | 1.3059 | −0.0043 | 1 | 0 |
| WCM0_PM2 | 10 | 11784598 | 13629192 | −0.9317 | −0.9068 | 1.0761 | −0.0093 | 1 | 0 |
| WCM0_PM2 | 10 | 13633835 | 44866775 | −0.6544 | −0.6198 | 1.3018 | −3.00E−04 | 1 | 0 |
| WCM0_PM2 | 10 | 44868752 | 69366602 | −0.4147 | −0.3732 | 1.5408 | 0.0034 | 2 | 0 |
| WCM0_PM2 | 10 | 69407203 | 71139853 | −1.0382 | −1.0175 | 0.9981 | −0.0101 | 1 | 0 |
| WCM0_PM2 | 10 | 71142230 | 73767859 | −0.5171 | −0.4784 | 1.4316 | 0.004 | 1 | 0 |
| WCM0_PM2 | 10 | 73768113 | 76989377 | −0.8529 | −0.825 | 1.1365 | −0.0075 | 1 | 0 |
| WCM0_PM2 | 10 | 76993945 | 93576903 | −0.4726 | −0.4327 | 1.4778 | 0.004 | 1 | 0 |
| WCM0_PM2 | 10 | 93579339 | 94822686 | −0.8672 | −0.8398 | NA | NA | NA | NA |
| WCM0_PM2 | 10 | 94822698 | 98945399 | −0.6058 | −0.5697 | 1.3401 | 0.0074 | 1 | 0 |
| WCM0_PM2 | 10 | 98985868 | 99437681 | −0.8439 | −0.8157 | 1.1449 | −0.0086 | 1 | 0 |
| WCM0_PM2 | 10 | 99437822 | 101419751 | −0.4552 | −0.4147 | 1.4822 | 0.0181 | 1 | 0 |
| WCM0_PM2 | 10 | 101421333 | 102240909 | −0.8648 | −0.8374 | 1.1223 | −0.003 | 1 | 0 |
| WCM0_PM2 | 10 | 102241672 | 103534966 | −0.65 | −0.6153 | NA | NA | NA | NA |
| WCM0_PM2 | 10 | 103541462 | 105363545 | −0.8947 | −0.8683 | 1.0978 | −0.0022 | 1 | 0 |
| WCM0_PM2 | 10 | 105363568 | 115537174 | −0.4954 | −0.456 | 1.4514 | 0.0066 | 1 | 0 |
| WCM0_PM2 | 10 | 115540311 | 124221558 | −0.6305 | −0.5952 | 1.3218 | 0.0021 | 1 | 0 |
| WCM0_PM2 | 10 | 124248397 | 135373659 | −0.4366 | −0.3956 | 1.5173 | 0.003 | 2 | 0 |
| WCM0_PM2 | 11 | 193096 | 1776299 | −0.7381 | −0.7062 | 1.2203 | 0.0056 | 1 | 0 |
| WCM0_PM2 | 11 | 1778573 | 4389340 | −0.5592 | −0.5217 | 1.3906 | 0.0025 | 1 | 0 |
| WCM0_PM2 | 11 | 4406601 | 8123112 | −0.3063 | −0.262 | 1.6626 | 0.0052 | 2 | 0 |
| WCM0_PM2 | 11 | 8132293 | 8953834 | −0.5667 | −0.5294 | NA | NA | NA | NA |
| WCM0_PM2 | 11 | 8959370 | 10051332 | −0.8664 | −0.839 | 1.1251 | −0.0071 | 1 | 0 |
| WCM0_PM2 | 11 | 10052764 | 18195365 | −0.4416 | −0.4008 | 1.5089 | 0.006 | 2 | 0 |
| WCM0_PM2 | 11 | 18195688 | 18735396 | −0.7882 | −0.758 | 1.1902 | −0.0076 | 1 | 0 |
| WCM0_PM2 | 11 | 18735645 | 32460662 | −0.3688 | −0.3261 | 1.5969 | −0.0016 | 2 | 0 |
| WCM0_PM2 | 11 | 32605479 | 34668143 | −0.7297 | −0.6975 | 1.2403 | −0.007 | 1 | 0 |
| WCM0_PM2 | 11 | 34668240 | 45970516 | −0.3951 | −0.3531 | 1.5597 | 0.0061 | 2 | 0 |
| WCM0_PM2 | 11 | 45970984 | 48328644 | −0.898 | −0.8717 | 1.0964 | −0.0034 | 1 | 0 |
| WCM0_PM2 | 11 | 48346544 | 61026792 | −0.4698 | −0.4298 | 1.4746 | 0.0101 | 1 | 0 |
| WCM0_PM2 | 11 | 61031994 | 69488070 | −0.8219 | −0.7929 | 1.1628 | −0.0085 | 1 | 0 |
| WCM0_PM2 | 11 | 69489984 | 78277277 | 0.3167 | 0.3733 | 1.3032 | 1.2874 | 1 | 1 |
| WCM0_PM2 | 11 | 78279790 | 107422527 | 0.5051 | 0.5645 | 1.4878 | 1.4698 | 1 | 1 |
| WCM0_PM2 | 11 | 107423881 | 108398850 | 0.1316 | 0.1851 | 1.1716 | 1.1022 | 1 | 1 |
| WCM0_PM2 | 11 | 108409842 | 116767967 | 0.4131 | 0.4712 | 1.4172 | 1.3553 | 1 | 1 |
| WCM0_PM2 | 11 | 116798054 | 117150696 | −0.7617 | −0.7306 | NA | NA | NA | NA |
| WCM0_PM2 | 11 | 117151012 | 118656883 | 0.1414 | 0.195 | 1.2333 | 1.0562 | 1 | 1 |
| WCM0_PM2 | 11 | 118754640 | 119005169 | −0.8951 | −0.8687 | NA | NA | NA | NA |
| WCM0_PM2 | 11 | 119020673 | 119181170 | 0.2568 | 0.3125 | NA | NA | NA | NA |
| WCM0_PM2 | 11 | 119181461 | 134257469 | 0.5004 | 0.5597 | 1.4785 | 1.4695 | 1 | 1 |
| WCM0_PM2 | 12 | 247582 | 542453 | −0.4631 | −0.4229 | NA | NA | NA | NA |
| WCM0_PM2 | 12 | 547683 | 1702039 | −0.9693 | −0.9458 | 1.0403 | −0.0021 | 1 | 0 |
| WCM0_PM2 | 12 | 1702508 | 6232298 | −0.5358 | −0.4977 | 1.4223 | −0.0058 | 1 | 0 |
| WCM0_PM2 | 12 | 6309752 | 9020489 | −0.877 | −0.85 | 1.1127 | −0.0032 | 1 | 0 |
| WCM0_PM2 | 12 | 9020794 | 9458927 | −0.657 | −0.6225 | NA | NA | NA | NA |
| WCM0_PM2 | 12 | 9459772 | 9723417 | 0.1703 | 0.2245 | NA | NA | NA | NA |
| WCM0_PM2 | 12 | 9747940 | 14839086 | −0.6346 | −0.5994 | 1.3058 | 0.0143 | 1 | 0 |
| WCM0_PM2 | 12 | 14840934 | 25348051 | −0.4383 | −0.3974 | 1.5147 | 0.0038 | 2 | 0 |
| WCM0_PM2 | 12 | 25356926 | 31310984 | 0.4305 | 0.4888 | 1.4431 | 1.3634 | 1 | 1 |
| WCM0_PM2 | 12 | 31354073 | 33532788 | 0.0773 | 0.1298 | 1.0964 | 1.0919 | 1 | 1 |
| WCM0_PM2 | 12 | 33535468 | 40761613 | 0.5227 | 0.5823 | NA | NA | NA | NA |
| WCM0_PM2 | 12 | 41302217 | 48439192 | −0.3991 | −0.3572 | 1.5634 | −0.002 | 2 | 0 |
| WCM0_PM2 | 12 | 48440154 | 52200304 | −0.8348 | −0.8062 | 1.1523 | −0.0086 | 1 | 0 |
| WCM0_PM2 | 12 | 52200490 | 53345434 | −0.3889 | −0.3467 | 1.5689 | 0.0038 | 2 | 0 |
| WCM0_PM2 | 12 | 53345536 | 54069803 | −0.8917 | −0.8652 | 1.107 | −0.009 | 1 | 0 |
| WCM0_PM2 | 12 | 54070026 | 56184384 | −0.5684 | −0.5312 | NA | NA | NA | NA |
| WCM0_PM2 | 12 | 56188221 | 57423088 | −0.943 | −0.9184 | 1.066 | −0.0078 | 1 | 0 |
| WCM0_PM2 | 12 | 57423197 | 70740147 | −0.6263 | −0.5908 | 1.3271 | 8.00E−04 | 1 | 0 |
| WCM0_PM2 | 12 | 70747582 | 93101445 | −0.4096 | −0.3679 | 1.5463 | 0.0035 | 2 | 0 |
| WCM0_PM2 | 12 | 93131252 | 93169934 | −0.5518 | −0.5141 | NA | NA | NA | NA |
| WCM0_PM2 | 12 | 93170658 | 102468197 | −0.7091 | −0.6763 | 1.2571 | −0.0055 | 1 | 0 |
| WCM0_PM2 | 12 | 102468798 | 109052704 | −0.5164 | −0.4777 | 1.4412 | −0.0049 | 1 | 0 |
| WCM0_PM2 | 12 | 109055826 | 110386627 | −0.7598 | −0.7287 | 1.213 | −0.0061 | 1 | 0 |
| WCM0_PM2 | 12 | 110389073 | 112846109 | −1.0103 | −0.9884 | NA | NA | NA | NA |
| WCM0_PM2 | 12 | 112846514 | 120208607 | −0.6454 | −0.6105 | 1.3168 | −0.0069 | 1 | 0 |
| WCM0_PM2 | 12 | 120210698 | 124270279 | −1.0063 | −0.9843 | 1.0198 | −0.0088 | 1 | 0 |
| WCM0_PM2 | 12 | 124272559 | 133779274 | −0.6314 | −0.5961 | 1.3298 | −0.0068 | 1 | 0 |
| WCM0_PM2 | 13 | 19705470 | 19969295 | −0.5235 | −0.4849 | NA | NA | NA | NA |
| WCM0_PM2 | 13 | 19971966 | 33349222 | −0.6452 | −0.6104 | 1.317 | −0.0069 | 1 | 0 |
| WCM0_PM2 | 13 | 33590659 | 40229956 | −0.4166 | −0.3752 | 1.5435 | −0.0014 | 2 | 0 |
| WCM0_PM2 | 13 | 40233592 | 53298200 | −0.5766 | −0.5396 | 1.3751 | 8.00E−04 | 1 | 0 |
| WCM0_PM2 | 13 | 53307540 | 80911729 | −0.4451 | −0.4044 | 1.5074 | 0.0036 | 2 | 0 |
| WCM0_PM2 | 13 | 84453646 | 95226999 | 0.7035 | 0.7654 | NA | NA | NA | NA |
| WCM0_PM2 | 13 | 95228632 | 101287231 | 0.3783 | 0.4358 | 2.6983 | 0.0071 | 3 | 0 |
| WCM0_PM2 | 13 | 101287365 | 113333982 | 0.6338 | 0.6949 | 3.2415 | −0.004 | 3 | 0 |
| WCM0_PM2 | 13 | 113337594 | 114285948 | 0.4412 | 0.4997 | 2.8137 | 0.014 | 3 | 0 |
| WCM0_PM2 | 13 | 114287464 | 115089411 | 0.7047 | 0.7666 | 3.4058 | −0.0032 | 3 | 0 |
| WCM0_PM2 | 14 | 19553608 | 20647381 | 0.5405 | 0.6003 | 1.6252 | 1.407 | 2 | 1 |
| WCM0_PM2 | 14 | 20665627 | 22237322 | 0.2887 | 0.3449 | 1.27 | 1.27 | 1 | 1 |
| WCM0_PM2 | 14 | 22251211 | 23079740 | 0.5599 | 0.62 | 1.5602 | 1.5135 | 2 | 2 |
| WCM0_PM2 | 14 | 23235808 | 23746270 | 0.1162 | 0.1694 | 1.1727 | 1.0765 | 1 | 1 |
| WCM0_PM2 | 14 | 23777099 | 31598628 | 0.4127 | 0.4708 | 1.3971 | 1.3746 | 1 | 1 |
| WCM0_PM2 | 14 | 31602579 | 34419954 | 0.0442 | 0.0961 | NA | NA | NA | NA |
| WCM0_PM2 | 14 | 34904457 | 35871226 | −0.5796 | −0.5427 | NA | NA | NA | NA |
| WCM0_PM2 | 14 | 35871727 | 50069115 | −0.073 | −0.0235 | 1.3393 | 0.6283 | 1 | 1 |
| WCM0_PM2 | 14 | 50074122 | 51080036 | −0.3612 | −0.3183 | NA | NA | NA | NA |
| WCM0_PM2 | 14 | 51081232 | 63856485 | −0.0678 | −0.0182 | 1.3161 | 0.6589 | 1 | 1 |
| WCM0_PM2 | 14 | 63858524 | 65035071 | −0.3643 | −0.3215 | NA | NA | NA | NA |
| WCM0_PM2 | 14 | 65041171 | 73418579 | −0.0856 | −0.0363 | 1.3077 | 0.6426 | 1 | 1 |
| WCM0_PM2 | 14 | 73420880 | 74707840 | −0.4792 | −0.4394 | 0.9527 | 0.5222 | 1 | 1 |
| WCM0_PM2 | 14 | 74711883 | 77818103 | −0.2283 | −0.1822 | 1.1889 | 0.5739 | 1 | 1 |
| WCM0_PM2 | 14 | 77843353 | 78288805 | −0.4023 | −0.3604 | 1.0603 | 0.4976 | 1 | 0 |
| WCM0_PM2 | 14 | 78325573 | 81994070 | 0.0155 | 0.0668 | NA | NA | NA | NA |
| WCM0_PM2 | 14 | 82000095 | 89082155 | 0.3685 | 0.4259 | NA | NA | NA | NA |
| WCM0_PM2 | 14 | 89082590 | 94089185 | 0.1862 | 0.2407 | 1.1863 | 1.1767 | 1 | 1 |
| WCM0_PM2 | 14 | 94097247 | 95267286 | 0.5257 | 0.5854 | 1.5415 | 1.4594 | 2 | 1 |
| WCM0_PM2 | 14 | 95556963 | 95599740 | −0.6165 | −0.5808 | NA | NA | NA | NA |
| WCM0_PM2 | 14 | 95658093 | 102276329 | 0.4049 | 0.4628 | 1.3908 | 1.3657 | 1 | 1 |
| WCM0_PM2 | 14 | 102323076 | 104552124 | 0.0325 | 0.0842 | 1.0709 | 1.0493 | 1 | 1 |
| WCM0_PM2 | 14 | 104559028 | 107283105 | 0.2789 | 0.3349 | 1.3472 | 1.1754 | 1 | 1 |
| WCM0_PM2 | 15 | 20169898 | 23932084 | −0.1619 | −0.1142 | 1.2317 | 0.616 | 1 | 1 |
| WCM0_PM2 | 15 | 23932260 | 28377413 | 0.1986 | 0.2532 | 1.5762 | 0.8076 | 2 | 1 |
| WCM0_PM2 | 15 | 28377971 | 40707140 | −0.0021 | 0.0489 | 1.3815 | 0.6874 | 1 | 1 |
| WCM0_PM2 | 15 | 40707588 | 42059254 | −0.4075 | −0.3658 | NA | NA | NA | NA |
| WCM0_PM2 | 15 | 42067557 | 42503974 | −0.0202 | 0.0304 | 1.3494 | 0.6931 | 1 | 1 |
| WCM0_PM2 | 15 | 42509989 | 45059999 | −0.2733 | −0.2282 | NA | NA | NA | NA |
| WCM0_PM2 | 15 | 45120750 | 45983200 | −0.0691 | −0.0195 | NA | NA | NA | NA |
| WCM0_PM2 | 15 | 47873582 | 50474900 | 0.1632 | 0.2172 | 1.5079 | 0.8171 | 2 | 1 |
| WCM0_PM2 | 15 | 50475141 | 63921025 | −0.0687 | −0.0191 | 1.3061 | 0.6675 | 1 | 1 |
| WCM0_PM2 | 15 | 63922860 | 66853490 | −0.3027 | −0.2584 | NA | NA | NA | NA |
| WCM0_PM2 | 15 | 66853613 | 74554874 | −0.0152 | 0.0355 | 1.3458 | 0.704 | 1 | 1 |
| WCM0_PM2 | 15 | 74559109 | 76303519 | −0.203 | −0.1563 | 1.1956 | 0.599 | 1 | 1 |
| WCM0_PM2 | 15 | 76426603 | 79237324 | −0.0695 | −0.0199 | 1.3175 | 0.6551 | 1 | 1 |
| WCM0_PM2 | 15 | 79254502 | 82431166 | 0.1529 | 0.2068 | 1.5464 | 0.7618 | 2 | 1 |
| WCM0_PM2 | 15 | 82443923 | 90119458 | −0.0493 | 7.00E−04 | 1.3221 | 0.6789 | 1 | 1 |
| WCM0_PM2 | 15 | 90126121 | 91560265 | −0.2636 | −0.2183 | 1.1324 | 0.5868 | 1 | 1 |
| WCM0_PM2 | 15 | 91565391 | 102359182 | 0.0905 | 0.1432 | 1.5183 | 0.6905 | 2 | 1 |
| WCM0_PM2 | 16 | 79642 | 449419 | −0.6082 | −0.5722 | 1.3385 | 0.0066 | 1 | 0 |
| WCM0_PM2 | 16 | 449680 | 19775443 | −0.8279 | −0.7991 | 1.158 | −0.0085 | 1 | 0 |
| WCM0_PM2 | 16 | 19800168 | 20990736 | −0.548 | −0.5102 | NA | NA | NA | NA |
| WCM0_PM2 | 16 | 20994049 | 28425387 | −0.7768 | −0.7462 | 1.2004 | −0.0081 | 1 | 0 |
| WCM0_PM2 | 16 | 28426211 | 31155110 | −0.9446 | −0.9201 | 1.0653 | −0.0083 | 1 | 0 |
| WCM0_PM2 | 16 | 31157089 | 31212879 | −0.8259 | −0.797 | NA | NA | NA | NA |
| WCM0_PM2 | 16 | 31213137 | 47292608 | −0.6745 | −0.6405 | 1.2881 | −0.0052 | 1 | 0 |
| WCM0_PM2 | 16 | 47294624 | 56701839 | −0.4839 | −0.4443 | 1.4661 | 0.0038 | 1 | 0 |
| WCM0_PM2 | 16 | 56703834 | 66757667 | −0.6955 | −0.6622 | 1.2712 | −0.0074 | 1 | 0 |
| WCM0_PM2 | 16 | 66759735 | 70605719 | −0.888 | −0.8613 | 1.1099 | −0.009 | 1 | 0 |
| WCM0_PM2 | 16 | 70680876 | 90162282 | −0.6486 | −0.6138 | 1.3139 | −0.0069 | 1 | 0 |
| WCM0_PM2 | 17 | 131595 | 5418817 | 0.0389 | 0.0907 | 1.0703 | 1.0594 | 1 | 1 |
| WCM0_PM2 | 17 | 5421236 | 7579791 | 0.2009 | 0.2556 | 1.1938 | 1.1938 | 1 | 1 |
| WCM0_PM2 | 17 | 7592190 | 7951862 | −0.7953 | −0.7654 | NA | NA | NA | NA |
| WCM0_PM2 | 17 | 7976196 | 10430347 | 0.186 | 0.2405 | 1.1922 | 1.1706 | 1 | 1 |
| WCM0_PM2 | 17 | 10431137 | 15687995 | 0.3593 | 0.4166 | 1.3442 | 1.3253 | 1 | 1 |
| WCM0_PM2 | 17 | 15848702 | 28029991 | 0.1934 | 0.248 | 1.2938 | 1.0812 | 1 | 1 |
| WCM0_PM2 | 17 | 28120952 | 28773049 | 0.7067 | 0.7686 | NA | NA | NA | NA |
| WCM0_PM2 | 17 | 28776701 | 31098162 | −0.8847 | −0.858 | 1.1056 | −0.0021 | 1 | 0 |
| WCM0_PM2 | 17 | 31099817 | 35345866 | −0.5523 | −0.5146 | 1.4031 | −0.0031 | 1 | 0 |
| WCM0_PM2 | 17 | 35346720 | 38600259 | −0.8732 | −0.846 | 1.1204 | −0.0077 | 1 | 0 |
| WCM0_PM2 | 17 | 38609360 | 39913897 | −0.5396 | −0.5015 | 1.4106 | 0.0021 | 1 | 0 |
| WCM0_PM2 | 17 | 39914791 | 39976609 | −0.7985 | −0.7687 | NA | NA | NA | NA |
| WCM0_PM2 | 17 | 39991535 | 43324269 | −0.9335 | −0.9086 | 1.0692 | −0.0038 | 1 | 0 |
| WCM0_PM2 | 17 | 43331781 | 47893178 | −0.7807 | −0.7502 | 1.1971 | −0.0081 | 1 | 0 |
| WCM0_PM2 | 17 | 47895371 | 56448242 | −0.5894 | −0.5528 | 1.3628 | 7.00E−04 | 1 | 0 |
| WCM0_PM2 | 17 | 56492800 | 59853906 | −0.8764 | −0.8494 | 1.1132 | −0.0031 | 1 | 0 |
| WCM0_PM2 | 17 | 59857753 | 60742221 | −0.07 | −0.0204 | NA | NA | NA | NA |
| WCM0_PM2 | 17 | 60743583 | 65074496 | 0.1673 | 0.2214 | 1.2667 | 1.065 | 1 | 1 |
| WCM0_PM2 | 17 | 65074640 | 66533866 | −0.0866 | −0.0374 | 1.1455 | 0.8033 | 1 | 1 |
| WCM0_PM2 | 17 | 66535469 | 70120419 | 0.3245 | 0.3812 | 1.4546 | 1.1503 | 1 | 1 |
| WCM0_PM2 | 17 | 70643814 | 72875693 | 0.1277 | 0.1811 | 1.2376 | 1.0299 | 1 | 1 |
| WCM0_PM2 | 17 | 72877182 | 75212689 | −0.0605 | −0.0107 | 1.0848 | 0.9004 | 1 | 1 |
| WCM0_PM2 | 17 | 75303303 | 81052264 | 0.1204 | 0.1737 | 1.2313 | 1.0246 | 1 | 1 |
| WCM0_PM2 | 18 | 47641 | 14613572 | 0.8405 | 0.9039 | 2.4557 | 1.2867 | 2 | 1 |
| WCM0_PM2 | 18 | 18539822 | 22023089 | −0.7818 | −0.7513 | 1.1962 | −0.0081 | 1 | 0 |
| WCM0_PM2 | 18 | 22025334 | 74980879 | −0.4804 | −0.4407 | 1.4713 | 0.0023 | 1 | 0 |
| WCM0_PM2 | 18 | 76753131 | 77287631 | 0.9451 | 1.0097 | NA | NA | NA | NA |
| WCM0_PM2 | 18 | 77439974 | 78005240 | −0.2433 | −0.1975 | NA | NA | NA | NA |
| WCM0_PM2 | 19 | 281360 | 652914 | 0.2311 | 0.2863 | 1.4055 | 1.0336 | 1 | 1 |
| WCM0_PM2 | 19 | 663030 | 5211578 | −0.0514 | −0.0015 | 1.0173 | 0.9806 | 1 | 1 |
| WCM0_PM2 | 19 | 5212166 | 6368687 | 0.0531 | 0.1051 | 1.0931 | 1.0581 | 1 | 1 |
| WCM0_PM2 | 19 | 6374267 | 15053073 | 0.4013 | 0.4592 | 1.7277 | 1.0218 | 2 | 1 |
| WCM0_PM2 | 19 | 15053274 | 16437806 | 0.6325 | 0.6935 | 2.0839 | 1.1505 | 2 | 1 |
| WCM0_PM2 | 19 | 16472746 | 19458053 | 0.4055 | 0.4634 | 1.7454 | 1.0122 | 2 | 1 |
| WCM0_PM2 | 19 | 19459774 | 36113658 | 0.6417 | 0.7028 | 2.1337 | 1.1217 | 2 | 1 |
| WCM0_PM2 | 19 | 36113819 | 36216177 | 0.5154 | 0.575 | NA | NA | NA | NA |
| WCM0_PM2 | 19 | 36216477 | 39664570 | 0.409 | 0.467 | 1.7652 | 0.9993 | 2 | 1 |
| WCM0_PM2 | 19 | 39665690 | 40487134 | 0.6412 | 0.7024 | 2.1149 | 1.1395 | 2 | 1 |
| WCM0_PM2 | 19 | 40504227 | 41807527 | 0.5236 | 0.5833 | 1.8521 | 1.1444 | 2 | 1 |
| WCM0_PM2 | 19 | 41808777 | 42599419 | 0.7377 | 0.8 | 2.1516 | 1.3307 | 2 | 1 |
| WCM0_PM2 | 19 | 42599702 | 42730593 | 0.1036 | 0.1566 | NA | NA | NA | NA |
| WCM0_PM2 | 19 | 42736706 | 42848981 | −5.3778 | −7.6442 | NA | NA | NA | NA |
| WCM0_PM2 | 19 | 42853725 | 44160626 | 0.1038 | 0.1568 | 2.2387 | −0.0091 | 2 | 0 |
| WCM0_PM2 | 19 | 44169475 | 45185806 | 0.7099 | 0.7719 | 2.1792 | 1.2358 | 2 | 1 |
| WCM0_PM2 | 19 | 45202505 | 48643216 | 0.4064 | 0.4644 | 1.762 | 0.9975 | 2 | 1 |
| WCM0_PM2 | 19 | 48646883 | 50983417 | 0.0868 | 0.1395 | 1.2081 | 0.9949 | 1 | 1 |
| WCM0_PM2 | 19 | 50983930 | 52543770 | 0.406 | 0.464 | 1.5767 | 1.182 | 2 | 1 |
| WCM0_PM2 | 19 | 52544743 | 56600103 | 0.2137 | 0.2686 | 1.397 | 1.0123 | 1 | 1 |
| WCM0_PM2 | 19 | 56600277 | 58721425 | 0.437 | 0.4955 | 1.7015 | 1.118 | 2 | 1 |
| WCM0_PM2 | 19 | 58723004 | 59082652 | 0.0969 | 0.1497 | 1.3014 | 0.9173 | 1 | 1 |
| WCM0_PM2 | 2 | 41620 | 8871156 | 0.6139 | 0.6748 | 1.6044 | 1.5882 | 2 | 2 |
| WCM0_PM2 | 2 | 8871342 | 15327042 | 0.2439 | 0.2993 | 1.2368 | 1.2243 | 1 | 1 |
| WCM0_PM2 | 2 | 15330456 | 23974938 | 0.1542 | 0.208 | 1.5066 | 0.8036 | 2 | 1 |
| WCM0_PM2 | 2 | 23977145 | 29221025 | −0.1946 | −0.1477 | 1.199 | 0.6064 | 1 | 1 |
| WCM0_PM2 | 2 | 29222093 | 31805938 | 0.1896 | 0.2441 | 1.5651 | 0.8037 | 2 | 1 |
| WCM0_PM2 | 2 | 32093595 | 32903910 | −0.3806 | −0.3382 | NA | NA | NA | NA |
| WCM0_PM2 | 2 | 32927910 | 39509625 | −0.0783 | −0.0288 | 1.2274 | 0.7331 | 1 | 1 |
| WCM0_PM2 | 2 | 39513990 | 42472779 | 0.4186 | 0.4768 | NA | NA | NA | NA |
| WCM0_PM2 | 2 | 42483762 | 44931420 | 0.1083 | 0.1613 | 1.1513 | 1.0853 | 1 | 1 |
| WCM0_PM2 | 2 | 44933438 | 61009909 | 0.3724 | 0.4298 | 1.3567 | 1.3375 | 1 | 1 |
| WCM0_PM2 | 2 | 61010358 | 62448923 | 0.0076 | 0.0588 | NA | NA | NA | NA |
| WCM0_PM2 | 2 | 62449434 | 69784043 | 0.4138 | 0.4718 | 1.3981 | 1.3756 | 1 | 1 |
| WCM0_PM2 | 2 | 69870168 | 70520818 | −0.8418 | −0.8135 | NA | NA | NA | NA |
| WCM0_PM2 | 2 | 70528100 | 72958333 | −0.4214 | −0.3801 | 1.5335 | 0.0033 | 2 | 0 |
| WCM0_PM2 | 2 | 72960203 | 75081407 | −0.7837 | −0.7534 | 1.181 | 0.0054 | 1 | 0 |
| WCM0_PM2 | 2 | 75094772 | 84800638 | −0.503 | −0.4639 | 1.4399 | 0.0102 | 1 | 0 |
| WCM0_PM2 | 2 | 84804548 | 85133195 | −0.6742 | −0.6402 | NA | NA | NA | NA |
| WCM0_PM2 | 2 | 85133552 | 87000551 | −0.9695 | −0.946 | 1.0391 | −0.001 | 1 | 0 |
| WCM0_PM2 | 2 | 87013039 | 90458605 | −0.6389 | −0.6038 | 1.2943 | 0.0217 | 1 | 0 |
| WCM0_PM2 | 2 | 91679084 | 102505246 | 0.1344 | 0.1879 | 1.2887 | 0.9895 | 1 | 1 |
| WCM0_PM2 | 2 | 102507619 | 108881794 | 0.371 | 0.4285 | 1.4086 | 1.283 | 1 | 1 |
| WCM0_PM2 | 2 | 108910203 | 113959008 | 0.0981 | 0.151 | 1.1804 | 1.0402 | 1 | 1 |
| WCM0_PM2 | 2 | 113976161 | 116101463 | −0.5792 | −0.5423 | NA | NA | NA | NA |
| WCM0_PM2 | 2 | 116257146 | 118578845 | 0.2226 | 0.2776 | NA | NA | NA | NA |
| WCM0_PM2 | 2 | 118579266 | 120020901 | 0.5646 | 0.6248 | NA | NA | NA | NA |
| WCM0_PM2 | 2 | 120059998 | 120690088 | 0.2745 | 0.3304 | NA | NA | NA | NA |
| WCM0_PM2 | 2 | 120692379 | 135309650 | −0.6521 | −0.6174 | 1.3033 | 4.00E−04 | 1 | 0 |
| WCM0_PM2 | 2 | 135470749 | 136872766 | −0.8725 | −0.8453 | 1.1123 | 9.00E−04 | 1 | 0 |
| WCM0_PM2 | 2 | 136873077 | 149520214 | −0.4934 | −0.4541 | 1.4571 | 0.0029 | 1 | 0 |
| WCM0_PM2 | 2 | 149522618 | 153006717 | 0.2567 | 0.3123 | 1.272 | 1.2114 | 1 | 1 |
| WCM0_PM2 | 2 | 153032111 | 170094604 | 0.503 | 0.5624 | 1.4842 | 1.4692 | 1 | 1 |
| WCM0_PM2 | 2 | 170096215 | 175432807 | 0.2795 | 0.3355 | 1.2785 | 1.2452 | 1 | 1 |
| WCM0_PM2 | 2 | 175436607 | 179394776 | 0.4669 | 0.5257 | 1.4601 | 1.4192 | 1 | 1 |
| WCM0_PM2 | 2 | 179395186 | 182763572 | 0.6046 | 0.6653 | 1.5956 | 1.5763 | 2 | 2 |
| WCM0_PM2 | 2 | 182763844 | 193059282 | 0.4695 | 0.5283 | 1.4759 | 1.4086 | 1 | 1 |
| WCM0_PM2 | 2 | 196544752 | 202901089 | 0.2746 | 0.3306 | 1.2716 | 1.2435 | 1 | 1 |
| WCM0_PM2 | 2 | 203066301 | 204732711 | −0.067 | −0.0173 | NA | NA | NA | NA |
| WCM0_PM2 | 2 | 204735510 | 218941149 | 0.4015 | 0.4594 | 1.3875 | 1.3624 | 1 | 1 |
| WCM0_PM2 | 2 | 218947871 | 219691643 | 0.1014 | 0.1543 | 1.131 | 1.0947 | 1 | 1 |
| WCM0_PM2 | 2 | 219692060 | 223918019 | 0.3654 | 0.4228 | 1.3609 | 1.3201 | 1 | 1 |
| WCM0_PM2 | 2 | 224462315 | 231406110 | 0.1389 | 0.1926 | 1.2545 | 1.0311 | 1 | 1 |
| WCM0_PM2 | 2 | 231406680 | 232458801 | −0.0634 | −0.0136 | 1.0229 | 0.9582 | 1 | 1 |
| WCM0_PM2 | 2 | 232573394 | 234344499 | −0.7659 | −0.735 | 1.2003 | 0.0013 | 1 | 0 |
| WCM0_PM2 | 2 | 234345003 | 234869664 | 0.2862 | 0.3423 | 1.4816 | 1.0539 | 1 | 1 |
| WCM0_PM2 | 2 | 234871984 | 241817549 | 0.464 | 0.5228 | 1.4528 | 1.4207 | 1 | 1 |
| WCM0_PM2 | 2 | 241826477 | 242815483 | 0.2499 | 0.3054 | 1.2584 | 1.2132 | 1 | 1 |
| WCM0_PM2 | 20 | 68336 | 2444510 | 0.5053 | 0.5647 | 1.4896 | 1.4686 | 1 | 1 |
| WCM0_PM2 | 20 | 2446414 | 6012026 | 0.2002 | 0.2549 | 1.2042 | 1.1823 | 1 | 1 |
| WCM0_PM2 | 20 | 6012706 | 26188912 | 0.5275 | 0.5872 | 1.5084 | 1.4962 | 2 | 1 |
| WCM0_PM2 | 20 | 29845502 | 35929672 | 0.0713 | 0.1237 | 1.0928 | 1.0862 | 1 | 1 |
| WCM0_PM2 | 20 | 35944804 | 45633522 | 0.3928 | 0.4505 | 1.379 | 1.354 | 1 | 1 |
| WCM0_PM2 | 20 | 45644838 | 52612578 | 0.1259 | 0.1793 | 1.1416 | 1.1231 | 1 | 1 |
| WCM0_PM2 | 20 | 52645076 | 55213066 | 0.3151 | 0.3717 | NA | NA | NA | NA |
| WCM0_PM2 | 20 | 55745967 | 60498779 | 0.5838 | 0.6442 | 1.593 | 1.5328 | 2 | 2 |
| WCM0_PM2 | 20 | 60499579 | 62076018 | 0.352 | 0.4091 | 1.3562 | 1.2996 | 1 | 1 |
| WCM0_PM2 | 20 | 62076743 | 62631145 | 0.1298 | 0.1832 | 1.147 | 1.1239 | 1 | 1 |
| WCM0_PM2 | 20 | 62632570 | 62904798 | 0.4445 | 0.5031 | NA | NA | NA | NA |
| WCM0_PM2 | 21 | 10862618 | 15481365 | −0.1172 | −0.0686 | 1.0861 | 0.8211 | 1 | 1 |
| WCM0_PM2 | 21 | 15516944 | 32503214 | 0.4724 | 0.5314 | 1.4717 | 1.4189 | 1 | 1 |
| WCM0_PM2 | 21 | 32508301 | 36088664 | 0.2095 | 0.2643 | 1.2157 | 1.1864 | 1 | 1 |
| WCM0_PM2 | 21 | 36164295 | 37507742 | −0.5712 | −0.5341 | NA | NA | NA | NA |
| WCM0_PM2 | 21 | 37510159 | 38538534 | 0.1265 | 0.1799 | 1.172 | 1.0936 | 1 | 1 |
| WCM0_PM2 | 21 | 38538740 | 44171297 | 0.4971 | 0.5563 | 1.4944 | 1.4467 | 1 | 1 |
| WCM0_PM2 | 21 | 44174134 | 45877032 | 0.277 | 0.333 | 1.3044 | 1.2148 | 1 | 1 |
| WCM0_PM2 | 21 | 45877279 | 48084181 | 0.5395 | 0.5993 | 1.5319 | 1.4981 | 2 | 1 |
| WCM0_PM2 | 22 | 16255826 | 29099483 | −0.6825 | −0.6487 | 1.2763 | −7.00E−04 | 1 | 0 |
| WCM0_PM2 | 22 | 29107915 | 32546979 | −0.8693 | −0.8419 | 1.1177 | −0.0019 | 1 | 0 |
| WCM0_PM2 | 22 | 32548092 | 37875356 | −0.6799 | −0.6461 | 1.2786 | −6.00E−04 | 1 | 0 |
| WCM0_PM2 | 22 | 37876231 | 40825786 | −0.8472 | −0.819 | 1.1353 | −0.0016 | 1 | 0 |
| WCM0_PM2 | 22 | 40827415 | 43460234 | −1.0345 | −1.0136 | 1.0007 | −0.0101 | 1 | 0 |
| WCM0_PM2 | 22 | 43464594 | 51216452 | −0.6855 | −0.6519 | 1.2801 | −0.0073 | 1 | 0 |
| WCM0_PM2 | 3 | 361508 | 9290657 | −0.3682 | −0.3255 | 1.5903 | 0.0058 | 2 | 0 |
| WCM0_PM2 | 3 | 9406821 | 10357067 | −0.7948 | −0.7649 | 1.1786 | −0.0015 | 1 | 0 |
| WCM0_PM2 | 3 | 10359786 | 31725644 | −0.4376 | −0.3967 | 1.5162 | 0.003 | 2 | 0 |
| WCM0_PM2 | 3 | 31744003 | 33704962 | −0.7497 | −0.7182 | NA | NA | NA | NA |
| WCM0_PM2 | 3 | 33725885 | 37458982 | −0.4893 | −0.4498 | 1.4561 | 0.0082 | 1 | 0 |
| WCM0_PM2 | 3 | 37476427 | 39230930 | −0.3093 | −0.2652 | 1.6582 | 0.006 | 2 | 0 |
| WCM0_PM2 | 3 | 39230976 | 47050777 | −0.4807 | −0.441 | 1.4709 | 0.0023 | 1 | 0 |
| WCM0_PM2 | 3 | 47058653 | 50214256 | −0.871 | −0.8437 | 1.1175 | −0.0031 | 1 | 0 |
| WCM0_PM2 | 3 | 50219843 | 53378889 | −0.5972 | −0.5608 | 1.3623 | −0.0064 | 1 | 0 |
| WCM0_PM2 | 3 | 53381491 | 56468749 | −0.314 | −0.2699 | 1.6536 | 0.0051 | 2 | 0 |
| WCM0_PM2 | 3 | 56468982 | 58520863 | −0.7424 | −0.7107 | 1.2292 | −0.0071 | 1 | 0 |
| WCM0_PM2 | 3 | 58552329 | 68802178 | −0.3797 | −0.3373 | 1.5872 | −0.0042 | 2 | 0 |
| WCM0_PM2 | 3 | 68929855 | 73657753 | −0.6067 | −0.5706 | 1.3525 | −0.0058 | 1 | 0 |
| WCM0_PM2 | 3 | 73673442 | 77526728 | 0.9528 | 1.0175 | NA | NA | NA | NA |
| WCM0_PM2 | 3 | 77530246 | 93761945 | 0.2885 | 0.3446 | NA | NA | NA | NA |
| WCM0_PM2 | 3 | 93768357 | 122103173 | −0.5391 | −0.501 | 1.4118 | 0.0014 | 1 | 0 |
| WCM0_PM2 | 3 | 122121623 | 122255103 | 0.2023 | 0.257 | NA | NA | NA | NA |
| WCM0_PM2 | 3 | 122255221 | 125690919 | 0.3715 | 0.4289 | 1.3667 | 1.3257 | 1 | 1 |
| WCM0_PM2 | 3 | 125694435 | 127413971 | 0.601 | 0.6617 | NA | NA | NA | NA |
| WCM0_PM2 | 3 | 127429452 | 141206043 | 0.348 | 0.405 | 1.3282 | 1.3201 | 1 | 1 |
| WCM0_PM2 | 3 | 141230995 | 142537157 | 0.0821 | 0.1346 | 1.109 | 1.0866 | 1 | 1 |
| WCM0_PM2 | 3 | 142539750 | 169487151 | 0.425 | 0.4832 | 1.405 | 1.3908 | 1 | 1 |
| WCM0_PM2 | 3 | 169492207 | 170198338 | 0.1104 | 0.1635 | 1.128 | 1.112 | 1 | 1 |
| WCM0_PM2 | 3 | 170198627 | 175520909 | 0.3676 | 0.425 | 1.3712 | 1.314 | 1 | 1 |
| WCM0_PM2 | 3 | 176743354 | 186572527 | 0.1134 | 0.1665 | 1.136 | 1.1086 | 1 | 1 |
| WCM0_PM2 | 3 | 186760652 | 194118667 | 0.4379 | 0.4964 | 1.4235 | 1.3978 | 1 | 1 |
| WCM0_PM2 | 3 | 194118875 | 195622217 | 0.1266 | 0.18 | 1.1456 | 1.1202 | 1 | 1 |
| WCM0_PM2 | 3 | 195778778 | 196666296 | −0.1561 | −0.1083 | 0.9324 | 0.9229 | 1 | 1 |
| WCM0_PM2 | 3 | 196669288 | 197847268 | 0.1403 | 0.1939 | 1.1846 | 1.1031 | 1 | 1 |
| WCM0_PM2 | 4 | 53403 | 3227431 | 0.1372 | 0.1908 | 1.1518 | 1.1309 | 1 | 1 |
| WCM0_PM2 | 4 | 3230443 | 25013932 | 0.4072 | 0.4651 | 1.3875 | 1.3735 | 1 | 1 |
| WCM0_PM2 | 4 | 25019600 | 38907405 | 0.2566 | 0.3122 | 1.2542 | 1.229 | 1 | 1 |
| WCM0_PM2 | 4 | 38910338 | 41270082 | −0.1185 | −0.07 | 0.9546 | 0.9507 | 1 | 1 |
| WCM0_PM2 | 4 | 41362921 | 44631587 | −0.6483 | −0.6135 | 1.3068 | 5.00E−04 | 1 | 0 |
| WCM0_PM2 | 4 | 44637911 | 47850328 | 0.4606 | 0.5194 | 1.4537 | 1.413 | 1 | 1 |
| WCM0_PM2 | 4 | 47853113 | 57976148 | 0.2775 | 0.3335 | 1.2831 | 1.237 | 1 | 1 |
| WCM0_PM2 | 4 | 57976304 | 71384483 | 0.4799 | 0.5389 | 1.4735 | 1.4323 | 1 | 1 |
| WCM0_PM2 | 4 | 71388451 | 71895121 | 0.1101 | 0.1632 | NA | NA | NA | NA |
| WCM0_PM2 | 4 | 72102288 | 73615956 | −0.518 | −0.4793 | NA | NA | NA | NA |
| WCM0_PM2 | 4 | 73923971 | 75971400 | 0.4167 | 0.4748 | NA | NA | NA | NA |
| WCM0_PM2 | 4 | 76407840 | 77305817 | 0.129 | 0.1825 | 1.1683 | 1.1013 | 1 | 1 |
| WCM0_PM2 | 4 | 77317514 | 83280653 | 0.4495 | 0.5081 | 1.4338 | 1.4107 | 1 | 1 |
| WCM0_PM2 | 4 | 83292671 | 84397844 | 0.0412 | 0.093 | 1.0688 | 1.0644 | 1 | 1 |
| WCM0_PM2 | 4 | 84403382 | 103912837 | 0.3641 | 0.4214 | 1.3487 | 1.3297 | 1 | 1 |
| WCM0_PM2 | 4 | 103947511 | 106158503 | −0.0202 | 0.0304 | NA | NA | NA | NA |
| WCM0_PM2 | 4 | 106290950 | 106474096 | −1.3231 | −1.3154 | NA | NA | NA | NA |
| WCM0_PM2 | 4 | 106510467 | 177073032 | 0.0185 | 0.0699 | 1.3417 | 0.7575 | 1 | 1 |
| WCM0_PM2 | 4 | 177077297 | 178911607 | −1.3584 | −1.3525 | NA | NA | NA | NA |
| WCM0_PM2 | 4 | 183090543 | 190947544 | −0.0713 | −0.0217 | 1.2897 | 0.6804 | 1 | 1 |
| WCM0_PM2 | 5 | 140598 | 16179313 | 0.5725 | 0.6328 | 1.5742 | 1.527 | 2 | 2 |
| WCM0_PM2 | 5 | 16451937 | 32599095 | 0.2707 | 0.3265 | 1.2731 | 1.2349 | 1 | 1 |
| WCM0_PM2 | 5 | 32712279 | 36679851 | 0.5239 | 0.5835 | 1.5304 | 1.4666 | 2 | 1 |
| WCM0_PM2 | 5 | 36680713 | 37697749 | 0.1979 | 0.2525 | NA | NA | NA | NA |
| WCM0_PM2 | 5 | 37701236 | 54552360 | 0.4912 | 0.5504 | 1.4764 | 1.4526 | 1 | 1 |
| WCM0_PM2 | 5 | 54555771 | 57879089 | 0.1975 | 0.2522 | NA | NA | NA | NA |
| WCM0_PM2 | 5 | 57913379 | 61602285 | 0.0792 | 0.1318 | NA | NA | NA | NA |
| WCM0_PM2 | 5 | 61642953 | 70952542 | −0.1147 | −0.0661 | 1.2772 | 0.6333 | 1 | 1 |
| WCM0_PM2 | 5 | 71015298 | 74016240 | 0.1103 | 0.1634 | 1.4894 | 0.7505 | 1 | 1 |
| WCM0_PM2 | 5 | 74016574 | 81548397 | −0.1189 | −0.0703 | 1.2686 | 0.6362 | 1 | 1 |
| WCM0_PM2 | 5 | 81549216 | 122950042 | 0.108 | 0.161 | 1.4699 | 0.7663 | 1 | 1 |
| WCM0_PM2 | 5 | 123966430 | 129072878 | −1.3823 | −1.3777 | 0.7621 | 0.0076 | 1 | 0 |
| WCM0_PM2 | 5 | 129240668 | 129521442 | −0.3597 | −0.3167 | NA | NA | NA | NA |
| WCM0_PM2 | 5 | 130495208 | 134347241 | −0.6855 | −0.6519 | 1.2795 | −0.0066 | 1 | 0 |
| WCM0_PM2 | 5 | 134364640 | 136957790 | −0.2075 | −0.1609 | 1.7865 | 0.0025 | 2 | 0 |
| WCM0_PM2 | 5 | 136961400 | 139189010 | 0.0576 | 0.1097 | 1.0856 | 1.0724 | 1 | 1 |
| WCM0_PM2 | 5 | 139189250 | 146741108 | 0.4133 | 0.4714 | 1.3864 | 1.3864 | 1 | 1 |
| WCM0_PM2 | 5 | 146750248 | 148810252 | 0.5864 | 0.6469 | 1.588 | 1.5436 | 2 | 2 |
| WCM0_PM2 | 5 | 148885195 | 170818440 | 0.4328 | 0.4912 | 1.4113 | 1.3999 | 1 | 1 |
| WCM0_PM2 | 5 | 170818808 | 180049837 | 0.2297 | 0.2849 | 1.2233 | 1.2134 | 1 | 1 |
| WCM0_PM2 | 5 | 180051068 | 180687760 | 0.5178 | 0.5774 | NA | NA | NA | NA |
| WCM0_PM2 | 6 | 292608 | 6654776 | 0.4794 | 0.5384 | 1.4657 | 1.4391 | 1 | 1 |
| WCM0_PM2 | 6 | 7182301 | 25435878 | 0.2811 | 0.3371 | 1.2838 | 1.2427 | 1 | 1 |
| WCM0_PM2 | 6 | 25450201 | 30314365 | 0.4215 | 0.4797 | 1.4058 | 1.3831 | 1 | 1 |
| WCM0_PM2 | 6 | 30314567 | 31948278 | 0.1778 | 0.2321 | 1.1829 | 1.1662 | 1 | 1 |
| WCM0_PM2 | 6 | 32006905 | 34101236 | 0.331 | 0.3878 | 1.3256 | 1.2911 | 1 | 1 |
| WCM0_PM2 | 6 | 34210575 | 37616830 | 0.1614 | 0.2154 | 1.167 | 1.1551 | 1 | 1 |
| WCM0_PM2 | 6 | 37617076 | 41318671 | 0.5233 | 0.5829 | 1.4979 | 1.4979 | 1 | 1 |
| WCM0_PM2 | 6 | 41533555 | 43748615 | 0.0753 | 0.1277 | 1.1059 | 1.0792 | 1 | 1 |
| WCM0_PM2 | 6 | 43749692 | 70993490 | 0.5045 | 0.5639 | 1.4887 | 1.4677 | 1 | 1 |
| WCM0_PM2 | 6 | 71004035 | 74530214 | 0.2601 | 0.3157 | NA | NA | NA | NA |
| WCM0_PM2 | 6 | 74533151 | 86200330 | 0.4421 | 0.5006 | 1.4578 | 1.3717 | 1 | 1 |
| WCM0_PM2 | 6 | 86200811 | 90718534 | 0.2223 | 0.2773 | NA | NA | NA | NA |
| WCM0_PM2 | 6 | 91226374 | 102516195 | 0.5204 | 0.58 | 1.5024 | 1.4873 | 2 | 1 |
| WCM0_PM2 | 6 | 105177519 | 111888889 | 0.2684 | 0.3242 | 1.2634 | 1.2405 | 1 | 1 |
| WCM0_PM2 | 6 | 111894128 | 134212899 | 0.5017 | 0.5611 | 1.4783 | 1.4724 | 1 | 1 |
| WCM0_PM2 | 6 | 134301398 | 139694619 | 0.3169 | 0.3735 | 1.3916 | 1.1995 | 1 | 1 |
| WCM0_PM2 | 6 | 139694903 | 149395251 | 0.5321 | 0.5918 | 1.5331 | 1.4812 | 2 | 1 |
| WCM0_PM2 | 6 | 149691155 | 151939199 | 0.1417 | 0.1954 | 1.1509 | 1.1392 | 1 | 1 |
| WCM0_PM2 | 6 | 152129234 | 154480960 | 0.453 | 0.5117 | 1.4257 | 1.4257 | 1 | 1 |
| WCM0_PM2 | 6 | 154481179 | 159651026 | 0.2834 | 0.3395 | 1.3013 | 1.2293 | 1 | 1 |
| WCM0_PM2 | 6 | 159652931 | 170893364 | 0.5518 | 0.6118 | 1.5313 | 1.5251 | 2 | 2 |
| WCM0_PM2 | 7 | 193518 | 4308362 | 0.2206 | 0.2756 | 1.2364 | 1.1847 | 1 | 1 |
| WCM0_PM2 | 7 | 4490854 | 6859544 | −0.0697 | −0.0201 | 1.0053 | 0.9671 | 1 | 1 |
| WCM0_PM2 | 7 | 7118608 | 22768361 | 0.5423 | 0.6021 | 1.5426 | 1.4934 | 2 | 1 |
| WCM0_PM2 | 7 | 22769134 | 24690101 | 0.2334 | 0.2886 | NA | NA | NA | NA |
| WCM0_PM2 | 7 | 24703212 | 27196139 | 0.5025 | 0.5618 | 1.53 | 1.4223 | 2 | 1 |
| WCM0_PM2 | 7 | 27203369 | 27689290 | −0.4371 | −0.3961 | NA | NA | NA | NA |
| WCM0_PM2 | 7 | 27702390 | 29160596 | 0.5652 | 0.6254 | NA | NA | NA | NA |
| WCM0_PM2 | 7 | 29394302 | 38398397 | −0.4228 | −0.3815 | 1.5289 | 0.0064 | 2 | 0 |
| WCM0_PM2 | 7 | 38402675 | 43496010 | 0.5485 | 0.6084 | NA | NA | NA | NA |
| WCM0_PM2 | 7 | 43503278 | 45217010 | 0.2961 | 0.3523 | 1.2818 | 1.2714 | 1 | 1 |
| WCM0_PM2 | 7 | 45222742 | 55273331 | 0.5964 | 0.657 | 1.5832 | 1.5704 | 2 | 2 |
| WCM0_PM2 | 7 | 55433718 | 77755197 | 0.0549 | 0.107 | 1.1285 | 1.0254 | 1 | 1 |
| WCM0_PM2 | 7 | 77756558 | 90896096 | 0.5341 | 0.5939 | 1.5216 | 1.4971 | 2 | 1 |
| WCM0_PM2 | 7 | 91503131 | 92207528 | 0.2702 | 0.326 | NA | NA | NA | NA |
| WCM0_PM2 | 7 | 92207673 | 97364142 | 0.5544 | 0.6144 | 1.5511 | 1.5108 | 2 | 2 |
| WCM0_PM2 | 7 | 97365604 | 99710913 | 0.0722 | 0.1246 | 1.0957 | 1.0846 | 1 | 1 |
| WCM0_PM2 | 7 | 99711211 | 102524002 | −0.0935 | −0.0444 | 1.1073 | 0.8321 | 1 | 1 |
| WCM0_PM2 | 7 | 102524683 | 102584917 | 0.1582 | 0.2121 | NA | NA | NA | NA |
| WCM0_PM2 | 7 | 102603959 | 102715726 | 0.4759 | 0.5349 | NA | NA | NA | NA |
| WCM0_PM2 | 7 | 102716221 | 104546621 | 0.3788 | 0.4363 | NA | NA | NA | NA |
| WCM0_PM2 | 7 | 104681442 | 105615426 | 0.0266 | 0.0782 | NA | NA | NA | NA |
| WCM0_PM2 | 7 | 105621503 | 107704435 | 0.3455 | 0.4026 | 1.3259 | 1.3178 | 1 | 1 |
| WCM0_PM2 | 7 | 107706389 | 127848017 | 0.5256 | 0.5853 | 1.5094 | 1.4912 | 2 | 1 |
| WCM0_PM2 | 7 | 127892124 | 131189125 | 0.157 | 0.2109 | 1.1822 | 1.1327 | 1 | 1 |
| WCM0_PM2 | 7 | 131190736 | 137686453 | 0.5076 | 0.567 | 1.4964 | 1.4664 | 1 | 1 |
| WCM0_PM2 | 7 | 137761363 | 141315352 | 0.0892 | 0.1419 | 1.1202 | 1.0865 | 1 | 1 |
| WCM0_PM2 | 7 | 141321512 | 144098992 | 0.6346 | 0.6957 | 1.6509 | 1.5885 | 2 | 2 |
| WCM0_PM2 | 7 | 144101738 | 149152452 | 0.1767 | 0.2309 | 1.2285 | 1.1187 | 1 | 1 |
| WCM0_PM2 | 7 | 149152770 | 150884185 | 0.4888 | 0.5479 | 1.4679 | 1.456 | 1 | 1 |
| WCM0_PM2 | 7 | 150884203 | 158707015 | 0.2909 | 0.3471 | 1.2836 | 1.2603 | 1 | 1 |
| WCM0_PM2 | 7 | 158711555 | 158937481 | 0.6668 | 0.7283 | NA | NA | NA | NA |
| WCM0_PM2 | 8 | 193740 | 2092737 | −4.8937 | −6.0467 | NA | NA | NA | NA |
| WCM0_PM2 | 8 | 2796211 | 21824547 | −0.5136 | −0.4748 | 1.4447 | −0.0056 | 1 | 0 |
| WCM0_PM2 | 8 | 21827056 | 22438145 | −0.7365 | −0.7045 | 1.2262 | 0.0011 | 1 | 0 |
| WCM0_PM2 | 8 | 22438929 | 28210114 | −0.5313 | −0.493 | 1.4254 | −0.0043 | 1 | 0 |
| WCM0_PM2 | 8 | 28210714 | 33454887 | −0.7347 | −0.7026 | 1.2366 | −0.0077 | 1 | 0 |
| WCM0_PM2 | 8 | 33827137 | 36703397 | −0.3591 | −0.3161 | NA | NA | NA | NA |
| WCM0_PM2 | 8 | 36721992 | 38705449 | 0.0878 | 0.1405 | 1.1359 | 1.0686 | 1 | 1 |
| WCM0_PM2 | 8 | 38775495 | 41791584 | 0.4065 | 0.4645 | 1.4358 | 1.3238 | 1 | 1 |
| WCM0_PM2 | 8 | 41792085 | 48889262 | 0.1395 | 0.1931 | NA | NA | NA | NA |
| WCM0_PM2 | 8 | 48920992 | 67342391 | 0.483 | 0.5421 | 1.4664 | 1.4457 | 1 | 1 |
| WCM0_PM2 | 8 | 67344765 | 68419129 | 0.1251 | 0.1785 | NA | NA | NA | NA |
| WCM0_PM2 | 8 | 68421844 | 95143138 | 0.4867 | 0.5458 | 1.4717 | 1.448 | 1 | 1 |
| WCM0_PM2 | 8 | 95158146 | 105601109 | 0.2965 | 0.3528 | 1.2874 | 1.2667 | 1 | 1 |
| WCM0_PM2 | 8 | 106430848 | 120430340 | −0.3757 | −0.3331 | 1.582 | 0.0056 | 2 | 0 |
| WCM0_PM2 | 8 | 120430496 | 131249188 | −0.6385 | −0.6034 | 1.3232 | −0.0068 | 1 | 0 |
| WCM0_PM2 | 8 | 131370420 | 144297217 | −0.3734 | −0.3308 | 1.5943 | −0.0041 | 2 | 0 |
| WCM0_PM2 | 8 | 144297402 | 145979616 | −0.7233 | −0.6909 | 1.2382 | 7.00E−04 | 1 | 0 |
| WCM0_PM2 | 8 | 145980050 | 146279475 | −0.411 | −0.3694 | NA | NA | NA | NA |
| WCM0_PM2 | 9 | 117666 | 3395411 | −0.817 | −0.7878 | 1.1609 | −0.0024 | 1 | 0 |
| WCM0_PM2 | 9 | 3828261 | 19032907 | 0.3081 | 0.3646 | 1.5072 | 1.0678 | 2 | 1 |
| WCM0_PM2 | 9 | 19049622 | 19622297 | −0.6964 | −0.6631 | NA | NA | NA | NA |
| WCM0_PM2 | 9 | 19785928 | 32989655 | −0.3181 | −0.2741 | 1.6464 | 0.0076 | 2 | 0 |
| WCM0_PM2 | 9 | 32989933 | 34520699 | −0.6726 | −0.6385 | NA | NA | NA | NA |
| WCM0_PM2 | 9 | 34521535 | 35906309 | −0.4402 | −0.3993 | 1.5181 | −0.0017 | 2 | 0 |
| WCM0_PM2 | 9 | 35906543 | 40293075 | −0.6037 | −0.5676 | NA | NA | NA | NA |
| WCM0_PM2 | 9 | 40772506 | 100881331 | 0.1458 | 0.1996 | 1.2816 | 1.0151 | 1 | 1 |
| WCM0_PM2 | 9 | 100887100 | 107456933 | 0.3459 | 0.403 | 1.3451 | 1.2994 | 1 | 1 |
| WCM0_PM2 | 9 | 107457275 | 108380338 | 0.0688 | 0.1212 | 1.1574 | 1.0178 | 1 | 1 |
| WCM0_PM2 | 9 | 108382293 | 114124331 | 0.2093 | 0.2642 | 1.2974 | 1.1045 | 1 | 1 |
| WCM0_PM2 | 9 | 114125923 | 116303615 | 0.0101 | 0.0613 | 1.1657 | 0.9211 | 1 | 1 |
| WCM0_PM2 | 9 | 116345746 | 123292368 | 0.4225 | 0.4807 | 1.5186 | 1.2723 | 2 | 1 |
| WCM0_PM2 | 9 | 123298630 | 127302473 | 0.1775 | 0.2317 | 1.2603 | 1.0882 | 1 | 1 |
| WCM0_PM2 | 9 | 127306061 | 130511499 | −0.0136 | 0.0371 | 1.0512 | 1.001 | 1 | 1 |
| WCM0_PM2 | 9 | 130511939 | 132889850 | −0.2186 | −0.1723 | 0.9619 | 0.813 | 1 | 1 |
| WCM0_PM2 | 9 | 132891001 | 135277524 | −0.0585 | −0.0087 | 1.0944 | 0.8936 | 1 | 1 |
| WCM0_PM2 | 9 | 135277822 | 139405781 | 0.1794 | 0.2337 | 1.2608 | 1.0909 | 1 | 1 |
| WCM0_PM2 | 9 | 139407452 | 140708975 | −0.0485 | 0.0016 | 1.0736 | 0.9286 | 1 | 1 |
| WCM0_PM2 | 9 | 140710405 | 141071438 | 0.4312 | 0.4895 | NA | NA | NA | NA |
| WCM0_PM2 | X | 2700163 | 15863538 | 0.4381 | 0.4965 | NA | NA | NA | NA |
| WCM0_PM2 | X | 15864026 | 24906098 | 0.2571 | 0.3127 | NA | NA | NA | NA |
| WCM0_PM2 | X | 24948626 | 40440276 | 0.5371 | 0.5969 | NA | NA | NA | NA |
| WCM0_PM2 | X | 40448260 | 49595646 | 0.1162 | 0.1694 | NA | NA | NA | NA |
| WCM0_PM2 | X | 49597153 | 69749713 | 0.4538 | 0.5124 | NA | NA | NA | NA |
| WCM0_PM2 | X | 69772057 | 70832671 | 0.0607 | 0.1129 | NA | NA | NA | NA |
| WCM0_PM2 | X | 70836225 | 77528378 | 0.3625 | 0.4198 | NA | NA | NA | NA |
| WCM0_PM2 | X | 77528664 | 99890621 | 0.5051 | 0.5645 | NA | NA | NA | NA |
| WCM0_PM2 | X | 99891701 | 100662873 | 0.1418 | 0.1955 | NA | NA | NA | NA |
| WCM0_PM2 | X | 100808315 | 112053144 | 0.582 | 0.6424 | NA | NA | NA | NA |
| WCM0_PM2 | X | 112054662 | 129546769 | 0.2154 | 0.2703 | NA | NA | NA | NA |
| WCM0_PM2 | X | 129629208 | 132092501 | 0.6263 | 0.6873 | NA | NA | NA | NA |
| WCM0_PM2 | X | 132092566 | 139040189 | 0.3661 | 0.4235 | NA | NA | NA | NA |
| WCM0_PM2 | X | 139047571 | 153033144 | 0.6676 | 0.7291 | NA | NA | NA | NA |
| WCM0_PM2 | X | 153040516 | 154509348 | 0.294 | 0.3502 | NA | NA | NA | NA |
| WCM0_T24 | 1 | 809089 | 2560735 | 0.1375 | −0.1435 | 0.9123 | 0.8984 | 1 | 1 |
| WCM0_T24 | 1 | 2938297 | 6266717 | 0.3447 | 0.0821 | 1.0643 | 1.0529 | 1 | 1 |
| WCM0_T24 | 1 | 6267467 | 11918318 | 0.1425 | −0.1381 | 0.9127 | 0.9048 | 1 | 1 |
| WCM0_T24 | 1 | 11918542 | 19609254 | −0.6828 | −1.074 | 0.9527 | −0.0027 | 1 | 0 |
| WCM0_T24 | 1 | 19611241 | 20517929 | 0.3917 | 0.1329 | 1.1592 | 1.0337 | 1 | 1 |
| WCM0_T24 | 1 | 20810127 | 28081886 | 0.1918 | −0.0841 | 0.9516 | 0.9351 | 1 | 1 |
| WCM0_T24 | 1 | 28086069 | 29424293 | −0.0094 | −0.3053 | NA | NA | NA | NA |
| WCM0_T24 | 1 | 29435902 | 29508193 | 0.1935 | −0.0822 | NA | NA | NA | NA |
| WCM0_T24 | 1 | 29520610 | 32280238 | 0.3222 | 0.0578 | 1.0578 | 1.0239 | 1 | 1 |
| WCM0_T24 | 1 | 32280378 | 33490000 | 0.0805 | −0.2062 | 0.8859 | 0.8478 | 1 | 1 |
| WCM0_T24 | 1 | 33502399 | 35370870 | 0.521 | 0.2719 | NA | NA | NA | NA |
| WCM0_T24 | 1 | 35452868 | 41249967 | 0.1914 | −0.0846 | 0.9451 | 0.941 | 1 | 1 |
| WCM0_T24 | 1 | 41282985 | 45116331 | 0.3722 | 0.1119 | 1.0993 | 1.0619 | 1 | 1 |
| WCM0_T24 | 1 | 45120411 | 46827456 | 0.1765 | −0.1009 | 0.9702 | 0.8948 | 1 | 1 |
| WCM0_T24 | 1 | 46860149 | 51787453 | 0.4195 | 0.1629 | 1.1521 | 1.0869 | 1 | 1 |
| WCM0_T24 | 1 | 51787790 | 53458951 | 0.1487 | −0.1313 | NA | NA | NA | NA |
| WCM0_T24 | 1 | 53480714 | 62149177 | 0.4335 | 0.1779 | 1.1532 | 1.1093 | 1 | 1 |
| WCM0_T24 | 1 | 62160390 | 64011686 | 0.1996 | −0.0756 | 0.9966 | 0.9013 | 1 | 1 |
| WCM0_T24 | 1 | 64017391 | 91182794 | 0.4314 | 0.1757 | 1.1332 | 1.1259 | 1 | 1 |
| WCM0_T24 | 1 | 91382378 | 94027785 | −0.6639 | −1.0517 | 0.9743 | −0.0095 | 1 | 0 |
| WCM0_T24 | 1 | 94033045 | 109143339 | −0.4631 | −0.8175 | 1.1223 | 0.0125 | 1 | 0 |
| WCM0_T24 | 1 | 109148810 | 109964448 | −0.6691 | −1.0578 | 0.9773 | −0.0166 | 1 | 0 |
| WCM0_T24 | 1 | 109968982 | 113063095 | −0.3937 | −0.7377 | 1.1943 | 0.0051 | 1 | 0 |
| WCM0_T24 | 1 | 113068733 | 115488995 | −0.6403 | −1.0239 | 0.9785 | 0.0051 | 1 | 0 |
| WCM0_T24 | 1 | 115489944 | 120342507 | −0.3669 | −0.7071 | 1.1925 | 0.0327 | 1 | 0 |
| WCM0_T24 | 1 | 120345717 | 150044289 | −0.1372 | −0.4476 | 1.0138 | 0.4527 | 1 | 0 |
| WCM0_T24 | 1 | 150048377 | 151237379 | −0.3397 | −0.6761 | NA | NA | NA | NA |
| WCM0_T24 | 1 | 151237661 | 151774461 | −0.1484 | −0.4601 | NA | NA | NA | NA |
| WCM0_T24 | 1 | 151774824 | 153520245 | 0.1807 | −0.0962 | 1.2401 | 0.6308 | 1 | 1 |
| WCM0_T24 | 1 | 153520352 | 156777124 | −0.1787 | −0.4941 | 0.9483 | 0.4717 | 1 | 0 |
| WCM0_T24 | 1 | 156779210 | 156816589 | 0.0331 | −0.2584 | NA | NA | NA | NA |
| WCM0_T24 | 1 | 156818845 | 160784472 | 0.1245 | −0.1577 | 1.1974 | 0.5955 | 1 | 1 |
| WCM0_T24 | 1 | 160786372 | 161476342 | −0.1701 | −0.4844 | NA | NA | NA | NA |
| WCM0_T24 | 1 | 161479615 | 178515005 | 0.1058 | −0.1783 | 1.1778 | 0.5896 | 1 | 1 |
| WCM0_T24 | 1 | 178716396 | 181021581 | −0.069 | −0.3715 | 1.0155 | 0.5305 | 1 | 1 |
| WCM0_T24 | 1 | 181024368 | 198721329 | 0.0601 | −0.2286 | 1.1046 | 0.6024 | 1 | 1 |
| WCM0_T24 | 1 | 198721900 | 206782713 | −0.0476 | −0.3477 | 1.0023 | 0.5694 | 1 | 1 |
| WCM0_T24 | 1 | 206784606 | 209968613 | 0.151 | −0.1287 | NA | NA | NA | NA |
| WCM0_T24 | 1 | 209969902 | 226465519 | −0.0336 | −0.3322 | 1.012 | 0.5766 | 1 | 1 |
| WCM0_T24 | 1 | 226474132 | 233192953 | 0.3159 | 0.0509 | 1.0404 | 1.0314 | 1 | 1 |
| WCM0_T24 | 1 | 233225848 | 233394376 | −0.566 | −0.9369 | NA | NA | NA | NA |
| WCM0_T24 | 1 | 233394644 | 247582157 | 0.1699 | −0.108 | 0.933 | 0.9228 | 1 | 1 |
| WCM0_T24 | 1 | 247582347 | 249212577 | 0.387 | 0.1278 | 1.1056 | 1.0797 | 1 | 1 |
| WCM0_T24 | 10 | 226060 | 5496374 | −0.4195 | −0.7674 | 1.1574 | 0.0176 | 1 | 0 |
| WCM0_T24 | 10 | 5496457 | 44141480 | −0.6282 | −1.0097 | 0.9962 | −0.0029 | 1 | 0 |
| WCM0_T24 | 10 | 44866767 | 69281699 | −0.4538 | −0.8068 | 1.1242 | 0.0191 | 1 | 0 |
| WCM0_T24 | 10 | 69299327 | 71008324 | −0.9091 | −1.3459 | 0.8104 | −0.0235 | 1 | 0 |
| WCM0_T24 | 10 | 71008448 | 73822733 | −0.5091 | −0.8707 | 1.0787 | 0.0151 | 1 | 0 |
| WCM0_T24 | 10 | 73823909 | 76788362 | −0.7169 | −1.1145 | 0.9168 | 0.0069 | 1 | 0 |
| WCM0_T24 | 10 | 76788700 | 93582083 | −0.4867 | −0.8447 | 1.0989 | 0.0148 | 1 | 0 |
| WCM0_T24 | 10 | 93586871 | 99518939 | −0.6192 | −0.9991 | 1.0065 | −0.0059 | 1 | 0 |
| WCM0_T24 | 10 | 99527347 | 101163194 | −0.3969 | −0.7415 | 1.1633 | 0.0329 | 1 | 0 |
| WCM0_T24 | 10 | 101163614 | 105207136 | −0.7053 | −1.1008 | 0.9354 | −0.0029 | 1 | 0 |
| WCM0_T24 | 10 | 105209225 | 120460891 | −0.4871 | −0.8452 | 1.0998 | 0.0134 | 1 | 0 |
| WCM0_T24 | 10 | 120467052 | 123970523 | −0.6855 | −1.0773 | 0.9551 | −0.0073 | 1 | 0 |
| WCM0_T24 | 10 | 123970779 | 135373568 | −0.4482 | −0.8003 | 1.1415 | 0.0069 | 1 | 0 |
| WCM0_T24 | 11 | 193096 | 1606274 | −0.6978 | −1.0919 | 0.9313 | 0.007 | 1 | 0 |
| WCM0_T24 | 11 | 1606486 | 4406616 | −0.5142 | −0.8766 | 1.0846 | 0.0046 | 1 | 0 |
| WCM0_T24 | 11 | 4406920 | 8118404 | −0.3501 | −0.6879 | 1.2272 | 0.0142 | 1 | 0 |
| WCM0_T24 | 11 | 8118740 | 10215523 | −0.6474 | −1.0323 | 0.9888 | −0.0109 | 1 | 0 |
| WCM0_T24 | 11 | 10327314 | 18254013 | −0.438 | −0.7886 | 1.1502 | 0.0076 | 1 | 0 |
| WCM0_T24 | 11 | 18257434 | 18787364 | −0.7344 | −1.1352 | 0.9183 | −0.0078 | 1 | 0 |
| WCM0_T24 | 11 | 18793489 | 30921082 | −0.3539 | −0.6923 | 1.221 | 0.0168 | 1 | 0 |
| WCM0_T24 | 11 | 30922081 | 32610256 | −0.4789 | −0.8357 | NA | NA | NA | NA |
| WCM0_T24 | 11 | 32610550 | 33375041 | −0.763 | −1.1694 | NA | NA | NA | NA |
| WCM0_T24 | 11 | 33564200 | 45967678 | −0.4435 | −0.7949 | 1.1435 | 0.0092 | 1 | 0 |
| WCM0_T24 | 11 | 45970479 | 48177610 | −0.7773 | −1.1865 | 0.893 | −0.0143 | 1 | 0 |
| WCM0_T24 | 11 | 48181567 | 61026798 | −0.4983 | −0.8582 | 1.0798 | 0.0235 | 1 | 0 |
| WCM0_T24 | 11 | 61031975 | 69488068 | −0.6922 | −1.0852 | 0.9554 | −0.0127 | 1 | 0 |
| WCM0_T24 | 11 | 69490020 | 78285336 | 0.3058 | 0.04 | 1.0337 | 1.0225 | 1 | 1 |
| WCM0_T24 | 11 | 78369097 | 101454169 | 0.4382 | 0.183 | 1.1401 | 1.1303 | 1 | 1 |
| WCM0_T24 | 11 | 101762165 | 107422626 | 0.3567 | 0.0951 | 1.1097 | 1.0266 | 1 | 1 |
| WCM0_T24 | 11 | 107423847 | 108398873 | 0.1219 | −0.1606 | 0.9663 | 0.823 | 1 | 1 |
| WCM0_T24 | 11 | 108409859 | 116827732 | 0.3641 | 0.1031 | 1.0915 | 1.0567 | 1 | 1 |
| WCM0_T24 | 11 | 116968947 | 117150677 | −0.6868 | −1.0788 | NA | NA | NA | NA |
| WCM0_T24 | 11 | 117151012 | 118656770 | 0.1064 | −0.1777 | 0.9822 | 0.786 | 1 | 1 |
| WCM0_T24 | 11 | 118754701 | 119027624 | −0.7219 | −1.1204 | NA | NA | NA | NA |
| WCM0_T24 | 11 | 119029024 | 134257494 | 0.4017 | 0.1437 | 1.1083 | 1.1012 | 1 | 1 |
| WCM0_T24 | 12 | 247582 | 406301 | −0.4563 | −0.8097 | NA | NA | NA | NA |
| WCM0_T24 | 12 | 416214 | 1904477 | −0.786 | −1.197 | 0.8814 | −0.009 | 1 | 0 |
| WCM0_T24 | 12 | 1904945 | 6080749 | −0.4883 | −0.8466 | 1.0994 | 0.0127 | 1 | 0 |
| WCM0_T24 | 12 | 6085324 | 9242574 | −0.7296 | −1.1295 | 0.914 | 1.00E−04 | 1 | 0 |
| WCM0_T24 | 12 | 9243032 | 9458916 | −0.5777 | −0.9505 | NA | NA | NA | NA |
| WCM0_T24 | 12 | 9459772 | 9723401 | 0.0023 | −0.2924 | NA | NA | NA | NA |
| WCM0_T24 | 12 | 9748029 | 25347813 | −0.5064 | −0.8676 | 1.0802 | 0.0159 | 1 | 0 |
| WCM0_T24 | 12 | 25348051 | 31555611 | 0.3537 | 0.0919 | 1.0911 | 1.0404 | 1 | 1 |
| WCM0_T24 | 12 | 31562177 | 33022048 | 0.0587 | −0.2302 | 0.8601 | 0.845 | 1 | 1 |
| WCM0_T24 | 12 | 33030926 | 41831798 | 0.3274 | 0.0634 | NA | NA | NA | NA |
| WCM0_T24 | 12 | 41871666 | 49048205 | −0.4538 | −0.8068 | 1.1361 | 0.0072 | 1 | 0 |
| WCM0_T24 | 12 | 49048825 | 51852444 | −0.7323 | −1.1328 | 0.9225 | −0.0104 | 1 | 0 |
| WCM0_T24 | 12 | 51853774 | 53238275 | −0.4279 | −0.777 | 1.1455 | 0.0217 | 1 | 0 |
| WCM0_T24 | 12 | 53238536 | 54066360 | −0.7383 | −1.14 | 0.9139 | −0.0064 | 1 | 0 |
| WCM0_T24 | 12 | 54069803 | 56119575 | −0.5037 | −0.8644 | 1.0869 | 0.0117 | 1 | 0 |
| WCM0_T24 | 12 | 56119888 | 57317835 | −0.7774 | −1.1867 | 0.8824 | −0.0038 | 1 | 0 |
| WCM0_T24 | 12 | 57323333 | 72338463 | −0.5465 | −0.9141 | 1.0532 | 0.0082 | 1 | 0 |
| WCM0_T24 | 12 | 72343458 | 93101399 | −0.4115 | −0.7581 | 1.1745 | 0.008 | 1 | 0 |
| WCM0_T24 | 12 | 93131258 | 102494798 | −0.6339 | −1.0163 | 0.9955 | −0.0067 | 1 | 0 |
| WCM0_T24 | 12 | 102505843 | 108917274 | −0.5026 | −0.8631 | 1.0923 | 0.0073 | 1 | 0 |
| WCM0_T24 | 12 | 108918060 | 110340836 | −0.6488 | −1.034 | 0.9929 | −0.0162 | 1 | 0 |
| WCM0_T24 | 12 | 110341933 | 112910759 | −0.8715 | −1.3001 | NA | NA | NA | NA |
| WCM0_T24 | 12 | 112915680 | 120565723 | −0.6196 | −0.9995 | 0.9945 | 0.0058 | 1 | 0 |
| WCM0_T24 | 12 | 120567104 | 124387485 | −0.8465 | −1.2698 | 0.8482 | −0.0187 | 1 | 0 |
| WCM0_T24 | 12 | 124393840 | 133779285 | −0.6175 | −0.9971 | 1.0096 | −0.0076 | 1 | 0 |
| WCM0_T24 | 13 | 19705463 | 23939346 | −0.7052 | −1.1005 | 0.9427 | −0.01 | 1 | 0 |
| WCM0_T24 | 13 | 23942596 | 80911815 | −0.5128 | −0.875 | 1.0877 | 0.0028 | 1 | 0 |
| WCM0_T24 | 13 | 84453615 | 115089411 | 0.4437 | 0.189 | 2.2687 | 0.0112 | 2 | 0 |
| WCM0_T24 | 14 | 19553690 | 21623076 | 0.278 | 0.0098 | 1.009 | 1.0046 | 1 | 1 |
| WCM0_T24 | 14 | 21623435 | 21992629 | 0.1002 | −0.1844 | 0.8945 | 0.8655 | 1 | 1 |
| WCM0_T24 | 14 | 21992684 | 23073012 | 0.4061 | 0.1485 | 1.1203 | 1.0964 | 1 | 1 |
| WCM0_T24 | 14 | 23075356 | 23745071 | 0.1493 | −0.1306 | 0.9414 | 0.8854 | 1 | 1 |
| WCM0_T24 | 14 | 23745384 | 31381351 | 0.3595 | 0.0981 | 1.0762 | 1.0646 | 1 | 1 |
| WCM0_T24 | 14 | 31382817 | 34419794 | 0.054 | −0.2353 | 0.8757 | 0.8233 | 1 | 1 |
| WCM0_T24 | 14 | 34904500 | 36003864 | −0.3346 | −0.6703 | NA | NA | NA | NA |
| WCM0_T24 | 14 | 36004160 | 59107495 | −0.0376 | −0.3366 | 0.9492 | 0.6346 | 1 | 1 |
| WCM0_T24 | 14 | 59108413 | 63735880 | 0.1546 | −0.1248 | 1.0688 | 0.7655 | 1 | 1 |
| WCM0_T24 | 14 | 63747841 | 73198586 | −0.0458 | −0.3457 | 0.918 | 0.6559 | 1 | 1 |
| WCM0_T24 | 14 | 73220020 | 75643284 | −0.2168 | −0.5369 | 0.8088 | 0.5697 | 1 | 1 |
| WCM0_T24 | 14 | 75745750 | 83159060 | −0.0134 | −0.3097 | 0.965 | 0.6486 | 1 | 1 |
| WCM0_T24 | 14 | 86087909 | 93994954 | 0.1885 | −0.0877 | 0.9503 | 0.9318 | 1 | 1 |
| WCM0_T24 | 14 | 93995629 | 95236188 | 0.3623 | 0.1011 | 1.0911 | 1.054 | 1 | 1 |
| WCM0_T24 | 14 | 95236329 | 95599774 | −0.5888 | −0.9635 | NA | NA | NA | NA |
| WCM0_T24 | 14 | 95657975 | 102028718 | 0.3323 | 0.0687 | 1.0863 | 1.0113 | 1 | 1 |
| WCM0_T24 | 14 | 102228256 | 107283105 | 0.1201 | −0.1626 | 0.9032 | 0.8836 | 1 | 1 |
| WCM0_T24 | 15 | 20170122 | 40846175 | 0.067 | −0.2209 | 1.0593 | 0.6567 | 1 | 1 |
| WCM0_T24 | 15 | 40849445 | 42058927 | −0.2438 | −0.5674 | NA | NA | NA | NA |
| WCM0_T24 | 15 | 42059091 | 45954315 | −0.044 | −0.3437 | 0.9823 | 0.5938 | 1 | 1 |
| WCM0_T24 | 15 | 45962146 | 50475113 | 0.2078 | −0.0667 | 1.159 | 0.7507 | 1 | 1 |
| WCM0_T24 | 15 | 50489819 | 50596307 | −0.0257 | −0.3235 | NA | NA | NA | NA |
| WCM0_T24 | 15 | 50601880 | 51217361 | −0.3043 | −0.6359 | NA | NA | NA | NA |
| WCM0_T24 | 15 | 51221216 | 64411058 | 0.0235 | −0.269 | 1.0142 | 0.6456 | 1 | 1 |
| WCM0_T24 | 15 | 64415673 | 66853439 | −0.1683 | −0.4825 | NA | NA | NA | NA |
| WCM0_T24 | 15 | 66853692 | 74907946 | 0.0659 | −0.2221 | 1.0386 | 0.676 | 1 | 1 |
| WCM0_T24 | 15 | 74908121 | 75983085 | −0.1386 | −0.4492 | 0.8808 | 0.5842 | 1 | 1 |
| WCM0_T24 | 15 | 76016672 | 90173735 | 0.0431 | −0.2473 | 1.0247 | 0.6603 | 1 | 1 |
| WCM0_T24 | 15 | 90174824 | 91434738 | −0.1375 | −0.4479 | 0.8815 | 0.5847 | 1 | 1 |
| WCM0_T24 | 15 | 91434958 | 102359203 | 0.0919 | −0.1935 | 1.086 | 0.6629 | 1 | 1 |
| WCM0_T24 | 16 | 79642 | 3255134 | −0.7024 | −1.0973 | 0.943 | −0.0082 | 1 | 0 |
| WCM0_T24 | 16 | 3273929 | 19800221 | −0.7698 | −1.1775 | 0.8966 | −0.0124 | 1 | 0 |
| WCM0_T24 | 16 | 19838379 | 20873713 | −0.5277 | −0.8922 | NA | NA | NA | NA |
| WCM0_T24 | 16 | 20926870 | 31368419 | −0.8111 | −1.2272 | 0.8642 | −0.0098 | 1 | 0 |
| WCM0_T24 | 16 | 31368681 | 47294541 | −0.6939 | −1.0872 | 0.9759 | −0.0345 | 1 | 0 |
| WCM0_T24 | 16 | 47345354 | 56920379 | −0.4976 | −0.8574 | 1.0887 | 0.0153 | 1 | 0 |
| WCM0_T24 | 16 | 56920969 | 67335862 | −0.6599 | −1.047 | 0.9767 | −0.0087 | 1 | 0 |
| WCM0_T24 | 16 | 67337224 | 70698647 | −0.7819 | −1.1921 | 0.8909 | −0.0156 | 1 | 0 |
| WCM0_T24 | 16 | 70699009 | 90162224 | −0.6244 | −1.0052 | 1.0002 | −0.0039 | 1 | 0 |
| WCM0_T24 | 17 | 131595 | 456587 | 0.2164 | −0.0573 | NA | NA | NA | NA |
| WCM0_T24 | 17 | 463734 | 5352092 | 0.0491 | −0.2407 | 0.8482 | 0.8445 | 1 | 1 |
| WCM0_T24 | 17 | 5353639 | 7579795 | 0.1721 | −0.1056 | 0.9335 | 0.9253 | 1 | 1 |
| WCM0_T24 | 17 | 7592052 | 7951653 | −0.6556 | −1.042 | NA | NA | NA | NA |
| WCM0_T24 | 17 | 7976225 | 28030074 | 0.1362 | −0.1449 | 0.9847 | 0.8241 | 1 | 1 |
| WCM0_T24 | 17 | 28120978 | 28773006 | 0.6456 | 0.4052 | NA | NA | NA | NA |
| WCM0_T24 | 17 | 28776656 | 31094648 | −0.7676 | −1.1749 | 0.8986 | −0.0128 | 1 | 0 |
| WCM0_T24 | 17 | 31098144 | 33769270 | −0.491 | −0.8497 | NA | NA | NA | NA |
| WCM0_T24 | 17 | 33770937 | 38600272 | −0.7385 | −1.1402 | 0.9187 | −0.0113 | 1 | 0 |
| WCM0_T24 | 17 | 38609413 | 39778647 | −0.5143 | −0.8767 | 1.0676 | 0.0217 | 1 | 0 |
| WCM0_T24 | 17 | 39780482 | 39925402 | −0.686 | −1.0778 | NA | NA | NA | NA |
| WCM0_T24 | 17 | 39925678 | 43320554 | −0.8083 | −1.2238 | 0.8727 | −0.0164 | 1 | 0 |
| WCM0_T24 | 17 | 43321172 | 47904814 | −0.6735 | −1.0631 | 0.9633 | −0.0061 | 1 | 0 |
| WCM0_T24 | 17 | 47915967 | 56448442 | −0.5474 | −0.9152 | 1.0636 | −0.003 | 1 | 0 |
| WCM0_T24 | 17 | 56492800 | 59853772 | −0.7612 | −1.1673 | 0.9024 | −0.0119 | 1 | 0 |
| WCM0_T24 | 17 | 59857668 | 60749358 | 0.0304 | −0.2613 | NA | NA | NA | NA |
| WCM0_T24 | 17 | 60751507 | 65103296 | 0.2294 | −0.0431 | 0.9864 | 0.9547 | 1 | 1 |
| WCM0_T24 | 17 | 65103605 | 66535512 | 0.0021 | −0.2926 | 0.8426 | 0.7902 | 1 | 1 |
| WCM0_T24 | 17 | 66536027 | 70732854 | 0.3445 | 0.0819 | 1.0813 | 1.0355 | 1 | 1 |
| WCM0_T24 | 17 | 70845863 | 72951866 | 0.1855 | −0.091 | 0.9522 | 0.9255 | 1 | 1 |
| WCM0_T24 | 17 | 72954334 | 75212606 | 0.0461 | −0.244 | 0.8612 | 0.8276 | 1 | 1 |
| WCM0_T24 | 17 | 75303235 | 81052326 | 0.1844 | −0.0922 | 0.9576 | 0.9186 | 1 | 1 |
| WCM0_T24 | 18 | 47641 | 14613572 | 0.7001 | 0.4631 | 1.7288 | 1.0283 | 2 | 1 |
| WCM0_T24 | 18 | 18539786 | 21199513 | −0.7802 | −1.19 | NA | NA | NA | NA |
| WCM0_T24 | 18 | 21209911 | 74980829 | −0.4867 | −0.8447 | 1.0957 | 0.018 | 1 | 0 |
| WCM0_T24 | 18 | 76753131 | 77287610 | 0.776 | 0.5438 | NA | NA | NA | NA |
| WCM0_T24 | 18 | 77439974 | 78005267 | −0.3226 | −0.6566 | NA | NA | NA | NA |
| WCM0_T24 | 19 | 281360 | 6374822 | 0.0245 | −0.2679 | 0.8388 | 0.8223 | 1 | 1 |
| WCM0_T24 | 19 | 6375471 | 12792570 | 0.3421 | 0.0793 | 1.3264 | 0.7867 | 1 | 1 |
| WCM0_T24 | 19 | 12800085 | 16797077 | 0.4346 | 0.1792 | 1.4309 | 0.8335 | 1 | 1 |
| WCM0_T24 | 19 | 16799023 | 19459754 | 0.3501 | 0.088 | 1.34 | 0.7858 | 1 | 1 |
| WCM0_T24 | 19 | 19460048 | 36248136 | 0.4963 | 0.2455 | 1.4922 | 0.8788 | 1 | 1 |
| WCM0_T24 | 19 | 36249139 | 39667174 | 0.358 | 0.0965 | 1.3177 | 0.8206 | 1 | 1 |
| WCM0_T24 | 19 | 39668305 | 40486361 | 0.5675 | 0.3218 | 1.5774 | 0.9224 | 2 | 1 |
| WCM0_T24 | 19 | 40486587 | 41854387 | 0.4496 | 0.1952 | 1.4221 | 0.8677 | 1 | 1 |
| WCM0_T24 | 19 | 41858807 | 42598030 | 0.6505 | 0.4104 | 1.5952 | 1.0628 | 2 | 1 |
| WCM0_T24 | 19 | 42599534 | 42730600 | −0.0682 | −0.3706 | NA | NA | NA | NA |
| WCM0_T24 | 19 | 42734934 | 42853841 | −3.4128 | NA | NA | NA | NA | NA |
| WCM0_T24 | 19 | 42854504 | 44160724 | −0.036 | −0.3348 | 1.612 | −0.0262 | 2 | 0 |
| WCM0_T24 | 19 | 44169628 | 45176162 | 0.6238 | 0.3819 | 1.6338 | 0.9722 | 2 | 1 |
| WCM0_T24 | 19 | 45179668 | 48254769 | 0.4183 | 0.1616 | 1.409 | 0.828 | 1 | 1 |
| WCM0_T24 | 19 | 48255804 | 51305589 | 0.1216 | −0.161 | 0.9503 | 0.8385 | 1 | 1 |
| WCM0_T24 | 19 | 51322424 | 52724300 | 0.2959 | 0.0292 | 1.1201 | 0.9208 | 1 | 1 |
| WCM0_T24 | 19 | 52725357 | 56600168 | 0.1337 | −0.1477 | 1.0046 | 0.8007 | 1 | 1 |
| WCM0_T24 | 19 | 56600434 | 58718482 | 0.3943 | 0.1357 | 1.3002 | 0.8971 | 1 | 1 |
| WCM0_T24 | 19 | 58721414 | 59082666 | 0.0276 | −0.2645 | 0.8527 | 0.8123 | 1 | 1 |
| WCM0_T24 | 2 | 41658 | 9098734 | 0.4654 | 0.2123 | 1.1622 | 1.1548 | 1 | 1 |
| WCM0_T24 | 2 | 9143675 | 14774872 | 0.2506 | −0.02 | 0.9948 | 0.9776 | 1 | 1 |
| WCM0_T24 | 2 | 15307204 | 21266460 | 0.1081 | −0.1758 | 1.1566 | 0.614 | 1 | 1 |
| WCM0_T24 | 2 | 23974889 | 29240721 | −0.0965 | −0.4021 | 1.0132 | 0.5003 | 1 | 1 |
| WCM0_T24 | 2 | 29245138 | 31805995 | 0.1345 | −0.1468 | 1.2053 | 0.6013 | 1 | 1 |
| WCM0_T24 | 2 | 32093587 | 38976865 | −0.158 | −0.4709 | 0.9495 | 0.4935 | 1 | 0 |
| WCM0_T24 | 2 | 38977210 | 61108996 | 0.2787 | 0.0105 | 1.027 | 0.9876 | 1 | 1 |
| WCM0_T24 | 2 | 61118840 | 62449664 | 0.0654 | −0.2228 | NA | NA | NA | NA |
| WCM0_T24 | 2 | 62449976 | 69784099 | 0.3663 | 0.1055 | 1.0852 | 1.0666 | 1 | 1 |
| WCM0_T24 | 2 | 69870070 | 70692791 | −0.761 | −1.1671 | NA | NA | NA | NA |
| WCM0_T24 | 2 | 70890857 | 85108085 | −0.5476 | −0.9154 | 1.0535 | 0.0068 | 1 | 0 |
| WCM0_T24 | 2 | 85133241 | 91955350 | −0.7074 | −1.1032 | 0.9323 | −0.0013 | 1 | 0 |
| WCM0_T24 | 2 | 95537471 | 96781921 | 0.2659 | −0.0034 | NA | NA | NA | NA |
| WCM0_T24 | 2 | 96789787 | 113955292 | 0.1105 | −0.1732 | 1.0125 | 0.7613 | 1 | 1 |
| WCM0_T24 | 2 | 113955511 | 116257097 | −0.5148 | −0.8773 | NA | NA | NA | NA |
| WCM0_T24 | 2 | 116283497 | 118578844 | 0.1248 | −0.1574 | NA | NA | NA | NA |
| WCM0_T24 | 2 | 118579333 | 120690074 | 0.3128 | 0.0476 | 1.0839 | 0.9831 | 1 | 1 |
| WCM0_T24 | 2 | 120692522 | 149501207 | −0.6036 | −0.9808 | 1.0042 | 0.0092 | 1 | 0 |
| WCM0_T24 | 2 | 149511635 | 153378459 | 0.3054 | 0.0395 | 1.0489 | 1.0066 | 1 | 1 |
| WCM0_T24 | 2 | 153399253 | 170101159 | 0.4362 | 0.1809 | 1.1397 | 1.1275 | 1 | 1 |
| WCM0_T24 | 2 | 170101470 | 176835571 | 0.2745 | 0.0059 | 1.0238 | 0.9845 | 1 | 1 |
| WCM0_T24 | 2 | 176844588 | 182781201 | 0.5271 | 0.2785 | 1.2182 | 1.2078 | 1 | 1 |
| WCM0_T24 | 2 | 182783427 | 201682007 | 0.3616 | 0.1004 | 1.0802 | 1.0639 | 1 | 1 |
| WCM0_T24 | 2 | 201682722 | 204360065 | 0.1152 | −0.168 | 0.9136 | 0.8667 | 1 | 1 |
| WCM0_T24 | 2 | 204571465 | 219082176 | 0.3552 | 0.0935 | 1.0727 | 1.0612 | 1 | 1 |
| WCM0_T24 | 2 | 219090610 | 219619124 | 0.1161 | −0.167 | 0.8956 | 0.8858 | 1 | 1 |
| WCM0_T24 | 2 | 219646982 | 223918019 | 0.3506 | 0.0884 | 1.0862 | 1.0402 | 1 | 1 |
| WCM0_T24 | 2 | 224462331 | 232458168 | 0.0703 | −0.2174 | 0.9719 | 0.7484 | 1 | 1 |
| WCM0_T24 | 2 | 232458472 | 234345133 | −0.6856 | −1.0773 | 0.9764 | −0.0286 | 1 | 0 |
| WCM0_T24 | 2 | 234346085 | 234656640 | 0.1846 | −0.092 | 1.092 | 0.7844 | 1 | 1 |
| WCM0_T24 | 2 | 234669127 | 234835179 | 0.2648 | −0.0046 | NA | NA | NA | NA |
| WCM0_T24 | 2 | 234839396 | 242009627 | 0.3908 | 0.1319 | 1.1052 | 1.0863 | 1 | 1 |
| WCM0_T24 | 2 | 242011155 | 242815427 | 0.182 | −0.0948 | 0.9527 | 0.92 | 1 | 1 |
| WCM0_T24 | 20 | 68337 | 871050 | 0.2776 | 0.0093 | 1.0196 | 0.9934 | 1 | 1 |
| WCM0_T24 | 20 | 896672 | 2315853 | 0.5108 | 0.261 | 1.2138 | 1.1829 | 1 | 1 |
| WCM0_T24 | 20 | 2315957 | 5974987 | 0.2168 | −0.0568 | 0.9635 | 0.9593 | 1 | 1 |
| WCM0_T24 | 20 | 5987094 | 25485680 | 0.4355 | 0.1801 | 1.1439 | 1.122 | 1 | 1 |
| WCM0_T24 | 20 | 25491350 | 35869704 | 0.1221 | −0.1604 | 0.9056 | 0.884 | 1 | 1 |
| WCM0_T24 | 20 | 35879674 | 45362477 | 0.3168 | 0.0518 | 1.0444 | 1.0287 | 1 | 1 |
| WCM0_T24 | 20 | 45607224 | 55213066 | 0.1421 | −0.1385 | 0.9294 | 0.8876 | 1 | 1 |
| WCM0_T24 | 20 | 55746005 | 62076743 | 0.363 | 0.1019 | 1.0732 | 1.0732 | 1 | 1 |
| WCM0_T24 | 20 | 62078139 | 62904807 | 0.1452 | −0.1351 | 0.9256 | 0.8956 | 1 | 1 |
| WCM0_T24 | 21 | 10862618 | 11058227 | −0.3717 | −0.7126 | 0.6506 | 0.5698 | 1 | 1 |
| WCM0_T24 | 21 | 14917181 | 32502539 | 0.3583 | 0.0969 | 1.0775 | 1.0613 | 1 | 1 |
| WCM0_T24 | 21 | 32503187 | 36164297 | 0.2056 | −0.069 | 0.9679 | 0.9387 | 1 | 1 |
| WCM0_T24 | 21 | 36164574 | 37507740 | −0.6003 | −0.977 | NA | NA | NA | NA |
| WCM0_T24 | 21 | 37510213 | 38600529 | 0.1346 | −0.1467 | 0.9221 | 0.8845 | 1 | 1 |
| WCM0_T24 | 21 | 38604704 | 48084267 | 0.3497 | 0.0875 | 1.0671 | 1.0579 | 1 | 1 |
| WCM0_T24 | 22 | 16255826 | 31658796 | −0.642 | −1.0259 | 0.9825 | −2.00E−04 | 1 | 0 |
| WCM0_T24 | 22 | 31662004 | 32330370 | −0.8807 | −1.3114 | NA | NA | NA | NA |
| WCM0_T24 | 22 | 32334021 | 37914053 | −0.5936 | −0.9692 | 1.0249 | −0.0033 | 1 | 0 |
| WCM0_T24 | 22 | 37914992 | 39085079 | −0.8162 | −1.2332 | 0.8757 | −0.025 | 1 | 0 |
| WCM0_T24 | 22 | 39085368 | 40820409 | −0.6413 | −1.0251 | 0.9778 | 0.005 | 1 | 0 |
| WCM0_T24 | 22 | 40825784 | 43275156 | −0.8492 | −1.2731 | 0.8549 | −0.0274 | 1 | 0 |
| WCM0_T24 | 22 | 43278148 | 51216430 | −0.6435 | −1.0276 | 0.9848 | −0.0038 | 1 | 0 |
| WCM0_T24 | 3 | 361508 | 9146339 | −0.418 | −0.7657 | 1.1695 | 0.0069 | 1 | 0 |
| WCM0_T24 | 3 | 9166545 | 10320592 | −0.7376 | −1.1391 | 0.9236 | −0.0155 | 1 | 0 |
| WCM0_T24 | 3 | 10327522 | 47049677 | −0.4876 | −0.8458 | 1.1084 | 0.0044 | 1 | 0 |
| WCM0_T24 | 3 | 47050053 | 50153065 | −0.7602 | −1.1661 | 0.894 | −0.0028 | 1 | 0 |
| WCM0_T24 | 3 | 50153356 | 53531244 | −0.5574 | −0.9268 | 1.0544 | −0.0024 | 1 | 0 |
| WCM0_T24 | 3 | 53531501 | 56468768 | −0.3432 | −0.6801 | 1.2292 | 0.019 | 1 | 0 |
| WCM0_T24 | 3 | 56469066 | 58514714 | −0.6921 | −1.0851 | 0.9471 | −0.0043 | 1 | 0 |
| WCM0_T24 | 3 | 58516383 | 67548671 | −0.3725 | −0.7135 | 1.2133 | 0.0064 | 1 | 0 |
| WCM0_T24 | 3 | 67559239 | 73432597 | −0.5442 | −0.9114 | 1.068 | −0.0047 | 1 | 0 |
| WCM0_T24 | 3 | 73433005 | 73673834 | −0.2481 | −0.5722 | NA | NA | NA | NA |
| WCM0_T24 | 3 | 74313545 | 77147482 | 0.7493 | 0.5154 | NA | NA | NA | NA |
| WCM0_T24 | 3 | 77526693 | 93761909 | 0.1959 | −0.0797 | NA | NA | NA | NA |
| WCM0_T24 | 3 | 93768348 | 122103167 | −0.51 | −0.8718 | 1.0849 | 0.0081 | 1 | 0 |
| WCM0_T24 | 3 | 122121756 | 122247301 | 0.1292 | −0.1526 | NA | NA | NA | NA |
| WCM0_T24 | 3 | 122247503 | 128205673 | 0.3147 | 0.0496 | 1.0462 | 1.0237 | 1 | 1 |
| WCM0_T24 | 3 | 128292314 | 129183462 | 0.0752 | −0.2119 | 0.8777 | 0.8491 | 1 | 1 |
| WCM0_T24 | 3 | 129185754 | 134967242 | 0.3099 | 0.0444 | 1.057 | 1.0056 | 1 | 1 |
| WCM0_T24 | 3 | 134968219 | 138668493 | 0.1077 | −0.1763 | NA | NA | NA | NA |
| WCM0_T24 | 3 | 138669181 | 141235267 | 0.3984 | 0.1401 | NA | NA | NA | NA |
| WCM0_T24 | 3 | 141248602 | 142522933 | 0.0832 | −0.2031 | 0.9069 | 0.8304 | 1 | 1 |
| WCM0_T24 | 3 | 142523372 | 178921596 | 0.2995 | 0.0331 | 1.0399 | 1.0066 | 1 | 1 |
| WCM0_T24 | 3 | 178922274 | 186760562 | 0.1258 | −0.1563 | 0.9091 | 0.8855 | 1 | 1 |
| WCM0_T24 | 3 | 186760724 | 194118982 | 0.3666 | 0.1058 | 1.0924 | 1.0598 | 1 | 1 |
| WCM0_T24 | 3 | 194126732 | 197847227 | 0.0659 | −0.2221 | 0.9007 | 0.8139 | 1 | 1 |
| WCM0_T24 | 4 | 53403 | 337555 | 0.3156 | 0.0506 | NA | NA | NA | NA |
| WCM0_T24 | 4 | 338214 | 3208310 | 0.0627 | −0.2258 | 0.8655 | 0.8448 | 1 | 1 |
| WCM0_T24 | 4 | 3208583 | 38937372 | 0.2491 | −0.0217 | 0.9926 | 0.9776 | 1 | 1 |
| WCM0_T24 | 4 | 38942552 | 40832542 | −0.0999 | −0.4059 | 0.7918 | 0.7177 | 1 | 1 |
| WCM0_T24 | 4 | 40892342 | 41035343 | −0.7022 | −1.097 | NA | NA | NA | NA |
| WCM0_T24 | 4 | 41259149 | 41362963 | 0.3434 | 0.0807 | NA | NA | NA | NA |
| WCM0_T24 | 4 | 41496647 | 44631587 | −0.636 | −1.0189 | 0.9939 | −0.0069 | 1 | 0 |
| WCM0_T24 | 4 | 44638037 | 71510108 | 0.2737 | 0.0051 | 1.021 | 0.9861 | 1 | 1 |
| WCM0_T24 | 4 | 71510373 | 71895085 | 0.072 | −0.2155 | NA | NA | NA | NA |
| WCM0_T24 | 4 | 72102372 | 73615956 | −0.4862 | −0.8442 | NA | NA | NA | NA |
| WCM0_T24 | 4 | 73923934 | 75971465 | 0.3582 | 0.0967 | NA | NA | NA | NA |
| WCM0_T24 | 4 | 76407774 | 77305563 | 0.1524 | −0.1272 | 0.9296 | 0.9015 | 1 | 1 |
| WCM0_T24 | 4 | 77305818 | 83278631 | 0.386 | 0.1267 | 1.1001 | 1.0836 | 1 | 1 |
| WCM0_T24 | 4 | 83279947 | 84518079 | 0.0918 | −0.1937 | 0.8869 | 0.8619 | 1 | 1 |
| WCM0_T24 | 4 | 84518624 | 103947684 | 0.3198 | 0.0551 | 1.0446 | 1.0333 | 1 | 1 |
| WCM0_T24 | 4 | 103947754 | 106158262 | −0.0101 | −0.3061 | NA | NA | NA | NA |
| WCM0_T24 | 4 | 106158602 | 106510492 | −1.0823 | −1.5604 | NA | NA | NA | NA |
| WCM0_T24 | 4 | 106534619 | 177072960 | −0.0261 | −0.3238 | 1.0107 | 0.5872 | 1 | 1 |
| WCM0_T24 | 4 | 177077291 | 178911678 | −1.2754 | −1.8075 | NA | NA | NA | NA |
| WCM0_T24 | 4 | 183090538 | 183603120 | −0.1374 | −0.4478 | NA | NA | NA | NA |
| WCM0_T24 | 4 | 183609476 | 190947578 | −0.0746 | −0.3777 | 0.9881 | 0.5512 | 1 | 1 |
| WCM0_T24 | 5 | 140603 | 16474952 | 0.4515 | 0.1974 | 1.1639 | 1.1293 | 1 | 1 |
| WCM0_T24 | 5 | 16475290 | 54439513 | 0.3184 | 0.0536 | 1.0457 | 1.03 | 1 | 1 |
| WCM0_T24 | 5 | 54442663 | 57750710 | 0.0715 | −0.216 | 1.023 | 0.6988 | 1 | 1 |
| WCM0_T24 | 5 | 57751173 | 59893741 | −0.1041 | −0.4106 | NA | NA | NA | NA |
| WCM0_T24 | 5 | 59894964 | 68399818 | −0.2796 | −0.6078 | 0.7588 | 0.5536 | 1 | 1 |
| WCM0_T24 | 5 | 68400524 | 68710051 | −0.6652 | −1.0533 | NA | NA | NA | NA |
| WCM0_T24 | 5 | 68710104 | 80256726 | −0.2892 | −0.6187 | 0.7575 | 0.5449 | 1 | 1 |
| WCM0_T24 | 5 | 80256869 | 122950146 | −0.18 | −0.4955 | 0.8208 | 0.5978 | 1 | 1 |
| WCM0_T24 | 5 | 123966430 | 129072912 | −1.254 | −1.7796 | 0.5685 | 0.014 | 1 | 0 |
| WCM0_T24 | 5 | 129240742 | 135391326 | −0.5939 | −0.9695 | 1.0253 | −0.0039 | 1 | 0 |
| WCM0_T24 | 5 | 135392355 | 139060120 | −0.0924 | −0.3976 | 0.8124 | 0.7058 | 1 | 1 |
| WCM0_T24 | 5 | 139060275 | 140175562 | 0.0802 | −0.2065 | 0.9935 | 0.7398 | 1 | 1 |
| WCM0_T24 | 5 | 140175856 | 153792464 | 0.2244 | −0.0486 | 1.0765 | 0.8573 | 1 | 1 |
| WCM0_T24 | 5 | 153792616 | 169023745 | 0.1255 | −0.1566 | 1.0437 | 0.7505 | 1 | 1 |
| WCM0_T24 | 5 | 169025580 | 170626733 | 0.2593 | −0.0106 | 1.0802 | 0.9052 | 1 | 1 |
| WCM0_T24 | 5 | 170632548 | 180687804 | 0.0381 | −0.2528 | 0.9559 | 0.7225 | 1 | 1 |
| WCM0_T24 | 6 | 292598 | 16328479 | 0.3317 | 0.0681 | 1.0529 | 1.0438 | 1 | 1 |
| WCM0_T24 | 6 | 17283144 | 18264056 | 0.0779 | −0.209 | NA | NA | NA | NA |
| WCM0_T24 | 6 | 18264241 | 30314231 | 0.331 | 0.0673 | 1.0602 | 1.0353 | 1 | 1 |
| WCM0_T24 | 6 | 30314567 | 37443173 | 0.2186 | −0.0548 | 0.969 | 0.9564 | 1 | 1 |
| WCM0_T24 | 6 | 37443914 | 41658889 | 0.4043 | 0.1465 | 1.1224 | 1.0914 | 1 | 1 |
| WCM0_T24 | 6 | 41691703 | 43655479 | 0.1192 | −0.1636 | 0.9055 | 0.8801 | 1 | 1 |
| WCM0_T24 | 6 | 43738512 | 56819326 | 0.4246 | 0.1684 | 1.1298 | 1.1177 | 1 | 1 |
| WCM0_T24 | 6 | 56846620 | 112020832 | 0.2899 | 0.0227 | 1.0258 | 1.0059 | 1 | 1 |
| WCM0_T24 | 6 | 112021439 | 149395284 | 0.3657 | 0.1048 | 1.0823 | 1.0683 | 1 | 1 |
| WCM0_T24 | 6 | 149691121 | 163983034 | 0.2609 | −0.0088 | 0.9982 | 0.9896 | 1 | 1 |
| WCM0_T24 | 6 | 163984415 | 170893364 | 0.4421 | 0.1873 | 1.1496 | 1.1276 | 1 | 1 |
| WCM0_T24 | 7 | 193518 | 4876252 | 0.158 | −0.1211 | 0.9366 | 0.9024 | 1 | 1 |
| WCM0_T24 | 7 | 4899543 | 6859542 | −0.0803 | −0.3841 | 0.7816 | 0.7509 | 1 | 1 |
| WCM0_T24 | 7 | 7118575 | 27196044 | 0.4006 | 0.1425 | 1.1038 | 1.1038 | 1 | 1 |
| WCM0_T24 | 7 | 27203387 | 27689281 | −0.4076 | −0.7537 | NA | NA | NA | NA |
| WCM0_T24 | 7 | 27702390 | 29160490 | 0.4754 | 0.223 | NA | NA | NA | NA |
| WCM0_T24 | 7 | 29394264 | 38398397 | −0.4157 | −0.7629 | 1.1573 | 0.0212 | 1 | 0 |
| WCM0_T24 | 7 | 38402685 | 39504152 | 0.4585 | 0.2049 | NA | NA | NA | NA |
| WCM0_T24 | 7 | 39504290 | 45222729 | 0.2907 | 0.0235 | 1.0473 | 0.9856 | 1 | 1 |
| WCM0_T24 | 7 | 45222991 | 55560124 | 0.4517 | 0.1975 | 1.1554 | 1.1381 | 1 | 1 |
| WCM0_T24 | 7 | 55565292 | 77755197 | 0.0387 | −0.2522 | 0.9193 | 0.7599 | 1 | 1 |
| WCM0_T24 | 7 | 77756702 | 91622278 | 0.442 | 0.1871 | 1.1385 | 1.1385 | 1 | 1 |
| WCM0_T24 | 7 | 91623921 | 92164259 | 0.2341 | −0.038 | NA | NA | NA | NA |
| WCM0_T24 | 7 | 92166255 | 97482617 | 0.4785 | 0.2264 | 1.1774 | 1.1623 | 1 | 1 |
| WCM0_T24 | 7 | 97483858 | 102566778 | 0.0371 | −0.254 | 0.9441 | 0.7331 | 1 | 1 |
| WCM0_T24 | 7 | 102572373 | 107720220 | 0.2901 | 0.0229 | 1.0226 | 1.0094 | 1 | 1 |
| WCM0_T24 | 7 | 107732084 | 127847910 | 0.4312 | 0.1755 | 1.1294 | 1.1294 | 1 | 1 |
| WCM0_T24 | 7 | 127892228 | 129944401 | 0.1467 | −0.1335 | 0.9186 | 0.9046 | 1 | 1 |
| WCM0_T24 | 7 | 129945681 | 137686382 | 0.3732 | 0.1129 | 1.0942 | 1.0685 | 1 | 1 |
| WCM0_T24 | 7 | 137761322 | 140534703 | 0.1512 | −0.1285 | 0.9198 | 0.9098 | 1 | 1 |
| WCM0_T24 | 7 | 140549929 | 142836762 | 0.5145 | 0.265 | 1.5109 | 0.8924 | 2 | 1 |
| WCM0_T24 | 7 | 142880505 | 147600666 | 0.399 | 0.1408 | 1.1191 | 1.0858 | 1 | 1 |
| WCM0_T24 | 7 | 147675067 | 148951079 | 0.0842 | −0.2021 | 0.9104 | 0.8282 | 1 | 1 |
| WCM0_T24 | 7 | 148951208 | 158727118 | 0.357 | 0.0955 | 1.0753 | 1.0615 | 1 | 1 |
| WCM0_T24 | 7 | 158734619 | 158937400 | 0.5529 | 0.3062 | NA | NA | NA | NA |
| WCM0_T24 | 8 | 192901 | 2092872 | −3.0828 | −5.6794 | NA | NA | NA | NA |
| WCM0_T24 | 8 | 2796106 | 36703425 | −0.4983 | −0.8582 | 1.1008 | 0.0024 | 1 | 0 |
| WCM0_T24 | 8 | 36722020 | 36793429 | 0.6061 | 0.363 | NA | NA | NA | NA |
| WCM0_T24 | 8 | 37553447 | 38801285 | 0.1232 | −0.1592 | 0.9171 | 0.8739 | 1 | 1 |
| WCM0_T24 | 8 | 38803739 | 41805386 | 0.3475 | 0.0852 | 1.0803 | 1.0413 | 1 | 1 |
| WCM0_T24 | 8 | 41806889 | 49637377 | 0.1354 | −0.1458 | NA | NA | NA | NA |
| WCM0_T24 | 8 | 49641685 | 67372732 | 0.4146 | 0.1576 | 1.1263 | 1.1046 | 1 | 1 |
| WCM0_T24 | 8 | 67380528 | 68396915 | 0.1581 | −0.1209 | NA | NA | NA | NA |
| WCM0_T24 | 8 | 68419107 | 95178148 | 0.4153 | 0.1584 | 1.1184 | 1.1136 | 1 | 1 |
| WCM0_T24 | 8 | 95182589 | 104080896 | 0.292 | 0.025 | 1.0296 | 1.0053 | 1 | 1 |
| WCM0_T24 | 8 | 104153171 | 105601037 | 0.4583 | 0.2047 | NA | NA | NA | NA |
| WCM0_T24 | 8 | 106430998 | 120431590 | −0.3666 | −0.7068 | 1.2081 | 0.0173 | 1 | 0 |
| WCM0_T24 | 8 | 120435087 | 131370293 | −0.5379 | −0.9042 | 1.0749 | −0.0062 | 1 | 0 |
| WCM0_T24 | 8 | 131414204 | 143995671 | −0.3561 | −0.6948 | 1.2218 | 0.0138 | 1 | 0 |
| WCM0_T24 | 8 | 143996318 | 146106958 | −0.6053 | −0.9828 | 1.0171 | −0.0051 | 1 | 0 |
| WCM0_T24 | 8 | 146107242 | 146279416 | −0.4039 | −0.7495 | NA | NA | NA | NA |
| WCM0_T24 | 9 | 117666 | 3395519 | −0.8783 | −1.3084 | 0.8135 | −0.006 | 1 | 0 |
| WCM0_T24 | 9 | 3828312 | 19032907 | 0.2105 | −0.0637 | 1.1082 | 0.8055 | 1 | 1 |
| WCM0_T24 | 9 | 19049846 | 19622248 | −0.6775 | −1.0678 | NA | NA | NA | NA |
| WCM0_T24 | 9 | 19786068 | 32784601 | −0.3376 | −0.6737 | 1.2458 | 0.008 | 1 | 0 |
| WCM0_T24 | 9 | 32784838 | 34500770 | −0.5989 | −0.9753 | NA | NA | NA | NA |
| WCM0_T24 | 9 | 34501162 | 35957830 | −0.4131 | −0.76 | 1.1717 | 0.0092 | 1 | 0 |
| WCM0_T24 | 9 | 35958062 | 40784472 | −0.5705 | −0.9421 | 1.0507 | −0.0098 | 1 | 0 |
| WCM0_T24 | 9 | 44174446 | 116267746 | 0.1336 | −0.1478 | 1.0639 | 0.7413 | 1 | 1 |
| WCM0_T24 | 9 | 116268730 | 123232466 | 0.3138 | 0.0487 | 1.1857 | 0.8829 | 1 | 1 |
| WCM0_T24 | 9 | 123234011 | 127302367 | 0.1534 | −0.1261 | 1.0517 | 0.7809 | 1 | 1 |
| WCM0_T24 | 9 | 127306063 | 128113084 | −0.1997 | −0.5177 | NA | NA | NA | NA |
| WCM0_T24 | 9 | 128116966 | 131010236 | 0.0379 | −0.2531 | 0.9419 | 0.7363 | 1 | 1 |
| WCM0_T24 | 9 | 131012592 | 134404670 | −0.1024 | −0.4087 | 0.8701 | 0.6365 | 1 | 1 |
| WCM0_T24 | 9 | 134404845 | 139612197 | 0.1405 | −0.1402 | 1.0416 | 0.7731 | 1 | 1 |
| WCM0_T24 | 9 | 139616368 | 140695306 | 0.0022 | −0.2925 | 0.9213 | 0.7117 | 1 | 1 |
| WCM0_T24 | 9 | 140706038 | 141071438 | 0.1908 | −0.0852 | NA | NA | NA | NA |
| WCM0_T24 | X | 2700141 | 32519915 | 0.133 | −0.1485 | NA | NA | NA | NA |
| WCM0_T24 | X | 32536097 | 69871339 | 0.3556 | 0.0939 | NA | NA | NA | NA |
| WCM0_T24 | X | 69890292 | 70814199 | 0.1097 | −0.174 | NA | NA | NA | NA |
| WCM0_T24 | X | 70817857 | 117512337 | 0.4034 | 0.1455 | NA | NA | NA | NA |
| WCM0_T24 | X | 117521338 | 123231545 | 0.1724 | −0.1053 | NA | NA | NA | NA |
| WCM0_T24 | X | 123234404 | 154509348 | 0.3858 | 0.1266 | NA | NA | NA | NA |
| WCM0_T5 | 1 | 809001 | 1021415 | 0.3361 | 0.1036 | 1.1084 | 1.0405 | 1 | 1 |
| WCM0_T5 | 1 | 1022516 | 2560735 | 0.0302 | −0.2088 | 0.8715 | 0.8591 | 1 | 1 |
| WCM0_T5 | 1 | 2938283 | 6219601 | 0.3556 | 0.1235 | 1.1038 | 1.075 | 1 | 1 |
| WCM0_T5 | 1 | 6228219 | 11986198 | 0.039 | −0.1998 | 0.8778 | 0.8636 | 1 | 1 |
| WCM0_T5 | 1 | 11994910 | 19609254 | −0.8478 | −1.1155 | 0.9242 | −0.0011 | 1 | 0 |
| WCM0_T5 | 1 | 19611241 | 19746169 | 0.3732 | 0.1414 | NA | NA | NA | NA |
| WCM0_T5 | 1 | 19775419 | 26528034 | 0.0894 | −0.1481 | 0.9126 | 0.8923 | 1 | 1 |
| WCM0_T5 | 1 | 26528963 | 29447714 | −0.0902 | −0.3321 | 0.8147 | 0.774 | 1 | 1 |
| WCM0_T5 | 1 | 29448035 | 32279802 | 0.2146 | −0.0203 | 1.0182 | 0.9538 | 1 | 1 |
| WCM0_T5 | 1 | 32280246 | 33502357 | −0.1065 | −0.3488 | 0.7989 | 0.7715 | 1 | 1 |
| WCM0_T5 | 1 | 33547888 | 33613325 | 0.2332 | −0.0013 | NA | NA | NA | NA |
| WCM0_T5 | 1 | 33623952 | 35370962 | 0.4917 | 0.262 | NA | NA | NA | NA |
| WCM0_T5 | 1 | 35452889 | 41234971 | 0.0449 | −0.1937 | 0.8887 | 0.86 | 1 | 1 |
| WCM0_T5 | 1 | 41236518 | 45205559 | 0.2777 | 0.0441 | 1.052 | 1.01 | 1 | 1 |
| WCM0_T5 | 1 | 45206659 | 46874308 | 0.0481 | −0.1904 | 0.8988 | 0.8539 | 1 | 1 |
| WCM0_T5 | 1 | 46874770 | 51001095 | 0.3621 | 0.1301 | 1.1189 | 1.0698 | 1 | 1 |
| WCM0_T5 | 1 | 51005355 | 51767391 | 0.1392 | −0.0972 | NA | NA | NA | NA |
| WCM0_T5 | 1 | 51768230 | 53370473 | −0.0168 | −0.2569 | NA | NA | NA | NA |
| WCM0_T5 | 1 | 53370644 | 55207122 | 0.2083 | −0.0267 | 0.9997 | 0.9636 | 1 | 1 |
| WCM0_T5 | 1 | 55223425 | 61849064 | 0.4829 | 0.2531 | 1.1991 | 1.1845 | 1 | 1 |
| WCM0_T5 | 1 | 61869900 | 63294797 | 0.0972 | −0.1402 | 0.9306 | 0.8842 | 1 | 1 |
| WCM0_T5 | 1 | 63299695 | 91182733 | 0.4192 | 0.1883 | 1.1452 | 1.1336 | 1 | 1 |
| WCM0_T5 | 1 | 91382468 | 94017978 | −0.791 | −1.0563 | 0.9661 | −0.0044 | 1 | 0 |
| WCM0_T5 | 1 | 94027982 | 109158978 | −0.5243 | −0.7793 | 1.1676 | −0.0023 | 1 | 0 |
| WCM0_T5 | 1 | 109167270 | 110009434 | −0.7858 | −1.0509 | 0.9707 | −0.0053 | 1 | 0 |
| WCM0_T5 | 1 | 110009736 | 112998603 | −0.4749 | −0.7282 | 1.2028 | 0.0046 | 1 | 0 |
| WCM0_T5 | 1 | 112998944 | 115487552 | −0.7549 | −1.0187 | 0.9906 | −0.0034 | 1 | 0 |
| WCM0_T5 | 1 | 115488952 | 120056566 | −0.4448 | −0.6971 | 1.2381 | −0.0045 | 1 | 0 |
| WCM0_T5 | 1 | 120056873 | 150064093 | −0.2725 | −0.5194 | 0.9705 | 0.4248 | 1 | 0 |
| WCM0_T5 | 1 | 150064406 | 151238876 | −0.503 | −0.7573 | NA | NA | NA | NA |
| WCM0_T5 | 1 | 151239071 | 151746984 | −0.2796 | −0.5267 | NA | NA | NA | NA |
| WCM0_T5 | 1 | 151747256 | 153520247 | 0.1204 | −0.1165 | 1.1968 | 0.648 | 1 | 1 |
| WCM0_T5 | 1 | 153520352 | 154458574 | −0.4601 | −0.7129 | 0.8175 | 0.4027 | 1 | 0 |
| WCM0_T5 | 1 | 154459078 | 155231545 | −0.2556 | −0.5021 | 0.9168 | 0.4954 | 1 | 0 |
| WCM0_T5 | 1 | 155231927 | 155300288 | −0.4005 | −0.6514 | NA | NA | NA | NA |
| WCM0_T5 | 1 | 155307495 | 155904156 | −0.603 | −0.8608 | NA | NA | NA | NA |
| WCM0_T5 | 1 | 155911607 | 156777124 | −0.3075 | −0.5554 | 0.9026 | 0.4583 | 1 | 0 |
| WCM0_T5 | 1 | 156778982 | 157067754 | −0.0422 | −0.2829 | 1.0771 | 0.5667 | 1 | 1 |
| WCM0_T5 | 1 | 157068608 | 159906707 | 0.1497 | −0.0866 | 1.2564 | 0.6271 | 1 | 1 |
| WCM0_T5 | 1 | 159907546 | 160784472 | −0.0021 | −0.2418 | NA | NA | NA | NA |
| WCM0_T5 | 1 | 160786361 | 161476353 | −0.3305 | −0.5792 | NA | NA | NA | NA |
| WCM0_T5 | 1 | 161479710 | 173556910 | 0.038 | −0.2008 | 1.1346 | 0.6056 | 1 | 1 |
| WCM0_T5 | 1 | 173567210 | 174926669 | −0.2702 | −0.5171 | NA | NA | NA | NA |
| WCM0_T5 | 1 | 174927070 | 178252762 | 0.2356 | 0.0012 | 1.3113 | 0.6904 | 1 | 1 |
| WCM0_T5 | 1 | 178269164 | 180853153 | −0.208 | −0.453 | 0.9881 | 0.4729 | 1 | 0 |
| WCM0_T5 | 1 | 180885338 | 182027176 | 0.2046 | −0.0305 | NA | NA | NA | NA |
| WCM0_T5 | 1 | 182353805 | 185278271 | −0.0438 | −0.2846 | 1.0961 | 0.5458 | 1 | 1 |
| WCM0_T5 | 1 | 185278604 | 198828264 | 0.1303 | −0.1064 | 1.2185 | 0.6394 | 1 | 1 |
| WCM0_T5 | 1 | 199997036 | 205814681 | −0.1259 | −0.3687 | 1.0082 | 0.5408 | 1 | 1 |
| WCM0_T5 | 1 | 205817108 | 211276897 | 0.121 | −0.1158 | 1.1936 | 0.6521 | 1 | 1 |
| WCM0_T5 | 1 | 211280545 | 213436117 | −0.2037 | −0.4487 | 0.97 | 0.4955 | 1 | 0 |
| WCM0_T5 | 1 | 213445880 | 223957600 | 0.0507 | −0.1878 | 1.1879 | 0.568 | 1 | 1 |
| WCM0_T5 | 1 | 223958179 | 226128881 | −0.284 | −0.5312 | 0.9121 | 0.4718 | 1 | 0 |
| WCM0_T5 | 1 | 226173012 | 226465450 | −0.0154 | −0.2555 | NA | NA | NA | NA |
| WCM0_T5 | 1 | 226474132 | 233231571 | 0.2992 | 0.0661 | 1.0682 | 1.0255 | 1 | 1 |
| WCM0_T5 | 1 | 233270728 | 233394314 | −0.6959 | −0.9573 | NA | NA | NA | NA |
| WCM0_T5 | 1 | 233394653 | 234745258 | 0.3955 | 0.1642 | 1.1627 | 1.0783 | 1 | 1 |
| WCM0_T5 | 1 | 235277152 | 236767444 | −3.00E−04 | −0.24 | 0.8615 | 0.832 | 1 | 1 |
| WCM0_T5 | 1 | 236850018 | 240492808 | 0.3645 | 0.1325 | 1.1152 | 1.0772 | 1 | 1 |
| WCM0_T5 | 1 | 240493895 | 247611749 | 0.1968 | −0.0384 | 0.9837 | 0.9638 | 1 | 1 |
| WCM0_T5 | 1 | 247614407 | 249212577 | 0.5062 | 0.2767 | 1.2767 | 1.1462 | 1 | 1 |
| WCM0_T5 | 10 | 225960 | 5436261 | −0.487 | −0.7407 | 1.1901 | 0.0068 | 1 | 0 |
| WCM0_T5 | 10 | 5454638 | 11797455 | −0.7504 | −1.014 | 0.9953 | −0.0049 | 1 | 0 |
| WCM0_T5 | 10 | 11805339 | 13639535 | −1.0432 | −1.3202 | 0.8121 | −0.0111 | 1 | 0 |
| WCM0_T5 | 10 | 13642272 | 44141516 | −0.7185 | −0.9808 | 1.018 | −0.0046 | 1 | 0 |
| WCM0_T5 | 10 | 44866798 | 64574192 | −0.4311 | −0.6829 | 1.2405 | 0.0053 | 1 | 0 |
| WCM0_T5 | 10 | 64575585 | 71160713 | −1.0437 | −1.3207 | 0.8106 | −0.0099 | 1 | 0 |
| WCM0_T5 | 10 | 71164832 | 73826880 | −0.5634 | −0.8198 | 1.1347 | −0.0016 | 1 | 0 |
| WCM0_T5 | 10 | 73827451 | 76979129 | −0.8936 | −1.1634 | 0.9031 | −0.0101 | 1 | 0 |
| WCM0_T5 | 10 | 76980586 | 93579343 | −0.5343 | −0.7896 | 1.1595 | −0.0024 | 1 | 0 |
| WCM0_T5 | 10 | 93579698 | 103541535 | −0.7305 | −0.9933 | 1.0094 | −0.0047 | 1 | 0 |
| WCM0_T5 | 10 | 103542007 | 105792617 | −0.9015 | −1.1717 | 0.8961 | −0.0083 | 1 | 0 |
| WCM0_T5 | 10 | 105792729 | 120789726 | −0.5383 | −0.7938 | 1.1502 | 0.0035 | 1 | 0 |
| WCM0_T5 | 10 | 120790032 | 121693218 | −0.8399 | −1.1073 | NA | NA | NA | NA |
| WCM0_T5 | 10 | 122216862 | 135373655 | −0.5227 | −0.7776 | 1.169 | −0.0023 | 1 | 0 |
| WCM0_T5 | 11 | 193096 | 1905194 | −0.8028 | −1.0686 | 0.9486 | 0.0049 | 1 | 0 |
| WCM0_T5 | 11 | 1905538 | 4388699 | −0.613 | −0.8712 | 1.0991 | −0.0057 | 1 | 0 |
| WCM0_T5 | 11 | 4388909 | 8123112 | −0.3821 | −0.6324 | 1.2907 | −4.00E−04 | 1 | 0 |
| WCM0_T5 | 11 | 8132316 | 8936420 | −0.5952 | −0.8527 | NA | NA | NA | NA |
| WCM0_T5 | 11 | 8938820 | 10064374 | −0.9552 | −1.2278 | 0.864 | −0.0101 | 1 | 0 |
| WCM0_T5 | 11 | 10215503 | 16812763 | −0.4481 | −0.7005 | 1.2321 | −0.0013 | 1 | 0 |
| WCM0_T5 | 11 | 16816104 | 18764555 | −0.6853 | −0.9463 | 1.0416 | −0.0037 | 1 | 0 |
| WCM0_T5 | 11 | 18764866 | 32421568 | −0.3981 | −0.6488 | 1.2763 | −7.00E−04 | 1 | 0 |
| WCM0_T5 | 11 | 32438016 | 34489825 | −0.797 | −1.0626 | 0.967 | −0.0094 | 1 | 0 |
| WCM0_T5 | 11 | 34492464 | 45987190 | −0.4769 | −0.7302 | 1.2011 | 0.0045 | 1 | 0 |
| WCM0_T5 | 11 | 45991469 | 48632382 | −0.9474 | −1.2197 | 0.8562 | 0.0025 | 1 | 0 |
| WCM0_T5 | 11 | 49059114 | 57145237 | −0.4708 | −0.724 | 1.1994 | 0.0115 | 1 | 0 |
| WCM0_T5 | 11 | 57146251 | 57799371 | −0.8483 | −1.1161 | NA | NA | NA | NA |
| WCM0_T5 | 11 | 57886001 | 61026442 | −0.5285 | −0.7836 | 1.1582 | 0.0036 | 1 | 0 |
| WCM0_T5 | 11 | 61026668 | 69488069 | −0.8475 | −1.1153 | 0.9292 | −0.0059 | 1 | 0 |
| WCM0_T5 | 11 | 69489975 | 78282457 | 0.2795 | 0.0459 | 1.0397 | 1.025 | 1 | 1 |
| WCM0_T5 | 11 | 78285483 | 107205604 | 0.48 | 0.2501 | 1.1893 | 1.1893 | 1 | 1 |
| WCM0_T5 | 11 | 107207421 | 107424548 | 0.3327 | 0.1002 | NA | NA | NA | NA |
| WCM0_T5 | 11 | 107427631 | 108380929 | 0.0656 | −0.1725 | 0.8982 | 0.8764 | 1 | 1 |
| WCM0_T5 | 11 | 108381165 | 116797969 | 0.3557 | 0.1236 | 1.1083 | 1.0706 | 1 | 1 |
| WCM0_T5 | 11 | 116824796 | 117150629 | −0.8141 | −1.0804 | NA | NA | NA | NA |
| WCM0_T5 | 11 | 117150937 | 118656779 | −0.0946 | −0.3366 | 0.9917 | 0.5921 | 1 | 1 |
| WCM0_T5 | 11 | 118754701 | 119020778 | −0.9085 | −1.1789 | NA | NA | NA | NA |
| WCM0_T5 | 11 | 119020937 | 134257433 | 0.4266 | 0.1958 | 1.1535 | 1.1372 | 1 | 1 |
| WCM0_T5 | 12 | 247582 | 416158 | −0.3868 | −0.6372 | NA | NA | NA | NA |
| WCM0_T5 | 12 | 416786 | 1399139 | −0.8985 | −1.1685 | 0.8911 | −0.0013 | 1 | 0 |
| WCM0_T5 | 12 | 1481188 | 6078426 | −0.5044 | −0.7587 | 1.1897 | −0.0076 | 1 | 0 |
| WCM0_T5 | 12 | 6080796 | 9098964 | −0.8314 | −1.0984 | 0.935 | −9.00E−04 | 1 | 0 |
| WCM0_T5 | 12 | 9142297 | 9572689 | −0.6076 | −0.8656 | NA | NA | NA | NA |
| WCM0_T5 | 12 | 9577223 | 9723401 | 0.4119 | 0.1809 | NA | NA | NA | NA |
| WCM0_T5 | 12 | 9747937 | 25348033 | −0.5302 | −0.7854 | 1.1628 | −0.0024 | 1 | 0 |
| WCM0_T5 | 12 | 25357043 | 31307336 | 0.4072 | 0.176 | 1.139 | 1.1206 | 1 | 1 |
| WCM0_T5 | 12 | 31310937 | 33022048 | −0.0491 | −0.29 | 0.828 | 0.8079 | 1 | 1 |
| WCM0_T5 | 12 | 33030952 | 41831706 | 0.3443 | 0.112 | NA | NA | NA | NA |
| WCM0_T5 | 12 | 41871666 | 48866585 | −0.4873 | −0.741 | 1.1985 | −0.0018 | 1 | 0 |
| WCM0_T5 | 12 | 48866694 | 52082622 | −0.9138 | −1.1845 | 0.8865 | −0.0066 | 1 | 0 |
| WCM0_T5 | 12 | 52093398 | 53298695 | −0.4757 | −0.729 | 1.2021 | 0.0045 | 1 | 0 |
| WCM0_T5 | 12 | 53298769 | 54070026 | −0.9478 | −1.2201 | 0.869 | −0.0105 | 1 | 0 |
| WCM0_T5 | 12 | 54105720 | 56101335 | −0.5988 | −0.8565 | NA | NA | NA | NA |
| WCM0_T5 | 12 | 56101475 | 56182879 | −0.7342 | −0.9971 | NA | NA | NA | NA |
| WCM0_T5 | 12 | 56184305 | 57109792 | −0.992 | −1.2665 | NA | NA | NA | NA |
| WCM0_T5 | 12 | 57110037 | 57975336 | −0.7387 | −1.0018 | 1.0031 | −0.0043 | 1 | 0 |
| WCM0_T5 | 12 | 57975669 | 70149212 | −0.6443 | −0.9037 | 1.0728 | −0.0038 | 1 | 0 |
| WCM0_T5 | 12 | 70149472 | 92539210 | −0.4498 | −0.7023 | 1.2242 | 0.005 | 1 | 0 |
| WCM0_T5 | 12 | 92814801 | 102437898 | −0.7468 | −1.0102 | 1.0015 | −0.0085 | 1 | 0 |
| WCM0_T5 | 12 | 102439851 | 108926074 | −0.5631 | −0.8195 | 1.1361 | −0.0028 | 1 | 0 |
| WCM0_T5 | 12 | 108929147 | 110238604 | −0.7885 | −1.0536 | 0.9678 | −0.0043 | 1 | 0 |
| WCM0_T5 | 12 | 110240942 | 112893828 | −1.0856 | −1.3648 | NA | NA | NA | NA |
| WCM0_T5 | 12 | 112910754 | 120580794 | −0.7573 | −1.0211 | 0.9956 | −0.0101 | 1 | 0 |
| WCM0_T5 | 12 | 120582244 | 124305197 | −1.0728 | −1.3514 | 0.791 | −0.0072 | 1 | 0 |
| WCM0_T5 | 12 | 124311414 | 133779327 | −0.7403 | −1.0035 | 0.9972 | 4.00E−04 | 1 | 0 |
| WCM0_T5 | 13 | 19705463 | 19943641 | −0.5106 | −0.7651 | NA | NA | NA | NA |
| WCM0_T5 | 13 | 19969256 | 29275650 | −0.7057 | −0.9675 | 1.0326 | −0.0098 | 1 | 0 |
| WCM0_T5 | 13 | 29278054 | 30110154 | −0.3962 | −0.6469 | NA | NA | NA | NA |
| WCM0_T5 | 13 | 30341378 | 33329987 | −0.6315 | −0.8904 | 1.0753 | 0.0037 | 1 | 0 |
| WCM0_T5 | 13 | 33332301 | 40261667 | −0.4193 | −0.6707 | 1.2547 | 0.0016 | 1 | 0 |
| WCM0_T5 | 13 | 40261945 | 51287351 | −0.6425 | −0.9018 | 1.0742 | −0.0037 | 1 | 0 |
| WCM0_T5 | 13 | 51417704 | 80911803 | −0.4629 | −0.7158 | 1.2193 | −0.0015 | 1 | 0 |
| WCM0_T5 | 13 | 84453654 | 101320989 | 0.332 | 0.0995 | 2.1523 | −0.0095 | 2 | 0 |
| WCM0_T5 | 13 | 101322605 | 113153517 | 0.535 | 0.3061 | 2.468 | 0.0047 | 2 | 0 |
| WCM0_T5 | 13 | 113158371 | 115089411 | 0.3575 | 0.1254 | 2.1798 | 0.0018 | 2 | 0 |
| WCM0_T5 | 14 | 19553682 | 21623602 | 1.1725 | 0.9521 | 2.8047 | 1.0647 | 3 | 1 |
| WCM0_T5 | 14 | 21623811 | 21979330 | 0.8841 | 0.6603 | 2.2592 | 0.9017 | 2 | 1 |
| WCM0_T5 | 14 | 21990271 | 22134111 | 1.142 | 0.9213 | NA | NA | NA | NA |
| WCM0_T5 | 14 | 22167296 | 23067241 | 1.3624 | 1.1438 | 3.2153 | 1.2039 | 3 | 1 |
| WCM0_T5 | 14 | 23070659 | 23651943 | 0.9415 | 0.7184 | 2.4416 | 0.8491 | 2 | 1 |
| WCM0_T5 | 14 | 23745172 | 24046510 | 1.2389 | 1.0192 | 3.0155 | 1.038 | 3 | 1 |
| WCM0_T5 | 14 | 24108103 | 29237899 | 1.5778 | 1.3611 | 4.0265 | 1.1111 | 4 | 1 |
| WCM0_T5 | 14 | 30046610 | 31348059 | 1.1696 | 0.9491 | NA | NA | NA | NA |
| WCM0_T5 | 14 | 31348684 | 32142787 | 0.6932 | 0.4667 | NA | NA | NA | NA |
| WCM0_T5 | 14 | 32257048 | 34270162 | 1.0907 | 0.8694 | NA | NA | NA | NA |
| WCM0_T5 | 14 | 34395110 | 35872922 | −0.5843 | −0.8414 | NA | NA | NA | NA |
| WCM0_T5 | 14 | 35873672 | 50069112 | −0.0999 | −0.342 | 1.0334 | 0.5445 | 1 | 1 |
| WCM0_T5 | 14 | 50074195 | 51210989 | −0.363 | −0.6127 | NA | NA | NA | NA |
| WCM0_T5 | 14 | 51214795 | 57704033 | −0.101 | −0.3432 | 1.0358 | 0.5408 | 1 | 1 |
| WCM0_T5 | 14 | 57706296 | 58033208 | 0.0259 | −0.2132 | NA | NA | NA | NA |
| WCM0_T5 | 14 | 58036721 | 60759244 | 1.0565 | 0.8348 | 2.4277 | 1.1396 | 2 | 1 |
| WCM0_T5 | 14 | 60903757 | 63735843 | 0.3731 | 0.1414 | 1.1209 | 1.085 | 1 | 1 |
| WCM0_T5 | 14 | 63747800 | 65007698 | −0.3609 | −0.6105 | NA | NA | NA | NA |
| WCM0_T5 | 14 | 65007918 | 73198508 | −0.1076 | −0.3499 | 1.0205 | 0.5487 | 1 | 1 |
| WCM0_T5 | 14 | 73220020 | 75747539 | −0.4002 | −0.651 | 0.8379 | 0.4357 | 1 | 0 |
| WCM0_T5 | 14 | 75747906 | 78325640 | −0.244 | −0.4901 | 0.9405 | 0.4835 | 1 | 0 |
| WCM0_T5 | 14 | 78353438 | 88792740 | 0.0687 | −0.1693 | 1.1749 | 0.6036 | 1 | 1 |
| WCM0_T5 | 14 | 88852166 | 94171006 | −0.2024 | −0.4473 | 0.9678 | 0.499 | 1 | 0 |
| WCM0_T5 | 14 | 94173034 | 95236217 | 0.0831 | −0.1546 | 1.2089 | 0.5878 | 1 | 1 |
| WCM0_T5 | 14 | 95267286 | 95599718 | −0.6555 | −0.9153 | NA | NA | NA | NA |
| WCM0_T5 | 14 | 95657981 | 102252501 | 0.3893 | 0.1578 | 1.1372 | 1.094 | 1 | 1 |
| WCM0_T5 | 14 | 102276329 | 104565303 | −0.0048 | −0.2446 | 0.8527 | 0.8354 | 1 | 1 |
| WCM0_T5 | 14 | 104569928 | 107283105 | 0.2427 | 0.0084 | 1.0243 | 0.9873 | 1 | 1 |
| WCM0_T5 | 15 | 20170126 | 40764155 | 0.3101 | 0.0771 | 1.1539 | 0.956 | 1 | 1 |
| WCM0_T5 | 15 | 40764405 | 42059384 | −0.1217 | −0.3645 | NA | NA | NA | NA |
| WCM0_T5 | 15 | 42067530 | 42459122 | 0.3114 | 0.0785 | 1.0785 | 1.0333 | 1 | 1 |
| WCM0_T5 | 15 | 42459552 | 44487265 | 0.0898 | −0.1478 | NA | NA | NA | NA |
| WCM0_T5 | 15 | 44581345 | 45059648 | −0.0229 | −0.2632 | NA | NA | NA | NA |
| WCM0_T5 | 15 | 45059998 | 47873531 | 0.2331 | −0.0014 | NA | NA | NA | NA |
| WCM0_T5 | 15 | 48052088 | 50474900 | 0.5381 | 0.3092 | 1.2642 | 1.2138 | 1 | 1 |
| WCM0_T5 | 15 | 50475138 | 50546878 | 0.216 | −0.0189 | NA | NA | NA | NA |
| WCM0_T5 | 15 | 50549576 | 51216221 | −0.1982 | −0.443 | NA | NA | NA | NA |
| WCM0_T5 | 15 | 51217475 | 64204410 | 0.2561 | 0.0221 | 1.0299 | 1.0009 | 1 | 1 |
| WCM0_T5 | 15 | 64216919 | 66850156 | −0.0114 | −0.2514 | NA | NA | NA | NA |
| WCM0_T5 | 15 | 66850373 | 74709909 | 0.3137 | 0.0809 | 1.0792 | 1.0361 | 1 | 1 |
| WCM0_T5 | 15 | 74710242 | 76303530 | 0.0717 | −0.1663 | 0.9084 | 0.8738 | 1 | 1 |
| WCM0_T5 | 15 | 76426559 | 90119328 | 0.3043 | 0.0712 | 1.057 | 1.0442 | 1 | 1 |
| WCM0_T5 | 15 | 90119458 | 91549226 | 0.0496 | −0.1889 | 0.8916 | 0.8629 | 1 | 1 |
| WCM0_T5 | 15 | 91549586 | 102359178 | 0.3749 | 0.1431 | 1.1201 | 1.0886 | 1 | 1 |
| WCM0_T5 | 16 | 79642 | 3107601 | −0.8347 | −1.1018 | 0.9377 | −0.0058 | 1 | 0 |
| WCM0_T5 | 16 | 3108498 | 19721858 | −0.9429 | −1.215 | 0.8684 | −0.0069 | 1 | 0 |
| WCM0_T5 | 16 | 19722751 | 21060901 | −0.7026 | −0.9643 | 1.0273 | −0.0023 | 1 | 0 |
| WCM0_T5 | 16 | 21061232 | 25263400 | −0.9173 | −1.1882 | 0.8843 | −0.0066 | 1 | 0 |
| WCM0_T5 | 16 | 25264317 | 28123223 | −0.7756 | −1.0402 | NA | NA | NA | NA |
| WCM0_T5 | 16 | 28124410 | 31195343 | −1.0097 | −1.285 | 0.8317 | −0.0109 | 1 | 0 |
| WCM0_T5 | 16 | 31195646 | 47409828 | −0.8091 | −1.0752 | 0.9513 | −0.0021 | 1 | 0 |
| WCM0_T5 | 16 | 47462684 | 56865783 | −0.5842 | −0.8413 | 1.1222 | −0.0059 | 1 | 0 |
| WCM0_T5 | 16 | 56865950 | 67335871 | −0.8121 | −1.0783 | 0.9527 | −0.0056 | 1 | 0 |
| WCM0_T5 | 16 | 67337129 | 70605559 | −0.9955 | −1.2701 | 0.8366 | −0.0074 | 1 | 0 |
| WCM0_T5 | 16 | 70680877 | 74330799 | −0.6561 | −0.9159 | 1.0639 | −0.0039 | 1 | 0 |
| WCM0_T5 | 16 | 74334058 | 75690332 | −0.9403 | −1.2123 | 0.8745 | −0.0114 | 1 | 0 |
| WCM0_T5 | 16 | 76311574 | 90162297 | −0.7287 | −0.9914 | 1.0107 | −0.0047 | 1 | 0 |
| WCM0_T5 | 17 | 131595 | 5392795 | −0.0347 | −0.2752 | 0.8322 | 0.8204 | 1 | 1 |
| WCM0_T5 | 17 | 5405108 | 7579847 | 0.1231 | −0.1138 | 0.9374 | 0.911 | 1 | 1 |
| WCM0_T5 | 17 | 7591984 | 7951674 | −0.8109 | −1.0771 | NA | NA | NA | NA |
| WCM0_T5 | 17 | 7976192 | 10447216 | 0.1257 | −0.1111 | 0.9259 | 0.9259 | 1 | 1 |
| WCM0_T5 | 17 | 10447442 | 15644598 | 0.2943 | 0.0611 | 1.0613 | 1.0252 | 1 | 1 |
| WCM0_T5 | 17 | 15645289 | 28030023 | 0.1362 | −0.1004 | 1.0214 | 0.8441 | 1 | 1 |
| WCM0_T5 | 17 | 28120952 | 28773021 | 0.7056 | 0.4793 | NA | NA | NA | NA |
| WCM0_T5 | 17 | 28776721 | 30981676 | −0.9857 | −1.2599 | 0.8464 | −0.0112 | 1 | 0 |
| WCM0_T5 | 17 | 30986261 | 35545279 | −0.6683 | −0.9286 | 1.058 | −0.0073 | 1 | 0 |
| WCM0_T5 | 17 | 35545436 | 38634873 | −0.9327 | −1.2043 | 0.8788 | −0.0109 | 1 | 0 |
| WCM0_T5 | 17 | 38635981 | 39912145 | −0.6307 | −0.8896 | 1.0809 | −0.0014 | 1 | 0 |
| WCM0_T5 | 17 | 39912444 | 40841608 | −0.9844 | −1.2585 | 0.8432 | −0.0073 | 1 | 0 |
| WCM0_T5 | 17 | 40842225 | 41599434 | −1.1371 | −1.419 | 0.7597 | −0.0117 | 1 | 0 |
| WCM0_T5 | 17 | 41601216 | 47782506 | −0.9185 | −1.1894 | 0.8882 | −0.0112 | 1 | 0 |
| WCM0_T5 | 17 | 47783183 | 56438267 | −0.6746 | −0.9352 | 1.0446 | 0.0013 | 1 | 0 |
| WCM0_T5 | 17 | 56439912 | 59853803 | −0.9705 | −1.2439 | 0.8507 | −0.0062 | 1 | 0 |
| WCM0_T5 | 17 | 59857621 | 60705135 | −0.0654 | −0.3067 | NA | NA | NA | NA |
| WCM0_T5 | 17 | 60742004 | 65050071 | 0.152 | −0.0842 | 0.9558 | 0.9308 | 1 | 1 |
| WCM0_T5 | 17 | 65051180 | 66533846 | −0.068 | −0.3093 | 0.8409 | 0.7731 | 1 | 1 |
| WCM0_T5 | 17 | 66535514 | 70845930 | 0.3903 | 0.1589 | 1.1426 | 1.0903 | 1 | 1 |
| WCM0_T5 | 17 | 70943913 | 72959226 | 0.143 | −0.0934 | 0.9488 | 0.9258 | 1 | 1 |
| WCM0_T5 | 17 | 72959915 | 75303291 | −0.0334 | −0.2739 | 0.8364 | 0.8178 | 1 | 1 |
| WCM0_T5 | 17 | 75316468 | 81052243 | 0.1298 | −0.1069 | 0.94 | 0.9172 | 1 | 1 |
| WCM0_T5 | 18 | 47647 | 14613577 | 0.5695 | 0.3411 | 1.5254 | 1.008 | 2 | 1 |
| WCM0_T5 | 18 | 18539805 | 22017921 | −0.8623 | −1.1307 | 0.9233 | −0.0099 | 1 | 0 |
| WCM0_T5 | 18 | 22020543 | 74980817 | −0.5141 | −0.7687 | 1.17 | 0.0039 | 1 | 0 |
| WCM0_T5 | 18 | 76753131 | 77287627 | 0.5897 | 0.3617 | NA | NA | NA | NA |
| WCM0_T5 | 18 | 77439928 | 78005267 | −0.4781 | −0.7315 | NA | NA | NA | NA |
| WCM0_T5 | 19 | 281360 | 498729 | 0.2626 | 0.0287 | NA | NA | NA | NA |
| WCM0_T5 | 19 | 501743 | 2796089 | −0.0655 | −0.3067 | 0.8168 | 0.8002 | 1 | 1 |
| WCM0_T5 | 19 | 2799689 | 4960629 | −0.1702 | −0.4143 | 0.792 | 0.7088 | 1 | 1 |
| WCM0_T5 | 19 | 5032947 | 6368641 | −0.0264 | −0.2667 | 0.8593 | 0.8031 | 1 | 1 |
| WCM0_T5 | 19 | 6374213 | 6732035 | 0.2117 | −0.0232 | 1.196 | 0.7721 | 1 | 1 |
| WCM0_T5 | 19 | 6732241 | 14991514 | 0.3966 | 0.1653 | 1.4617 | 0.7811 | 1 | 1 |
| WCM0_T5 | 19 | 14991897 | 16296545 | 0.5823 | 0.3541 | 1.7067 | 0.8497 | 2 | 1 |
| WCM0_T5 | 19 | 16301381 | 19466525 | 0.3941 | 0.1627 | 1.4693 | 0.7695 | 1 | 1 |
| WCM0_T5 | 19 | 19478880 | 36303720 | 0.5897 | 0.3616 | 1.7326 | 0.8372 | 2 | 1 |
| WCM0_T5 | 19 | 36304011 | 39760191 | 0.4037 | 0.1725 | 1.4942 | 0.7598 | 1 | 1 |
| WCM0_T5 | 19 | 39760435 | 40487096 | 0.6451 | 0.4179 | 1.7158 | 0.9562 | 2 | 1 |
| WCM0_T5 | 19 | 40504314 | 41854387 | 0.5134 | 0.2841 | 1.5928 | 0.8425 | 2 | 1 |
| WCM0_T5 | 19 | 41858807 | 42599415 | 0.7607 | 0.5352 | 1.8782 | 1.02 | 2 | 1 |
| WCM0_T5 | 19 | 42599733 | 42730600 | 0.1168 | −0.1201 | NA | NA | NA | NA |
| WCM0_T5 | 19 | 42740978 | 42853637 | −5.1922 | −8.2362 | NA | NA | NA | NA |
| WCM0_T5 | 19 | 42854346 | 44160661 | 0.1232 | −0.1136 | 1.85 | −0.0015 | 2 | 0 |
| WCM0_T5 | 19 | 44169651 | 45206951 | 0.736 | 0.5101 | 1.8402 | 1.0081 | 2 | 1 |
| WCM0_T5 | 19 | 45207392 | 48640975 | 0.4497 | 0.2193 | 1.5231 | 0.8053 | 2 | 1 |
| WCM0_T5 | 19 | 48643400 | 50969439 | 0.1561 | −0.08 | 1.0823 | 0.8098 | 1 | 1 |
| WCM0_T5 | 19 | 50970897 | 52538609 | 0.4143 | 0.1833 | 1.3181 | 0.9528 | 1 | 1 |
| WCM0_T5 | 19 | 52543737 | 56599405 | 0.2298 | −0.0048 | 1.2042 | 0.7892 | 1 | 1 |
| WCM0_T5 | 19 | 56599668 | 58601554 | 0.375 | 0.1433 | 1.3373 | 0.8716 | 1 | 1 |
| WCM0_T5 | 19 | 58639382 | 59082678 | 0.0191 | −0.2201 | 0.9842 | 0.7328 | 1 | 1 |
| WCM0_T5 | 2 | 41623 | 9460492 | 0.4844 | 0.2546 | 1.2391 | 1.1469 | 1 | 1 |
| WCM0_T5 | 2 | 9463404 | 21266424 | 0.1294 | −0.1073 | 1.0791 | 0.7775 | 1 | 1 |
| WCM0_T5 | 2 | 23974884 | 29256420 | −0.2472 | −0.4934 | 0.9303 | 0.4904 | 1 | 0 |
| WCM0_T5 | 2 | 29258494 | 31805986 | 0.0926 | −0.1449 | 1.1735 | 0.6353 | 1 | 1 |
| WCM0_T5 | 2 | 32093571 | 32897428 | −0.4992 | −0.7533 | NA | NA | NA | NA |
| WCM0_T5 | 2 | 32903996 | 39008990 | −0.1662 | −0.4101 | 0.9504 | 0.5547 | 1 | 1 |
| WCM0_T5 | 2 | 39025555 | 60983586 | 0.2216 | −0.0131 | 1.0091 | 0.9727 | 1 | 1 |
| WCM0_T5 | 2 | 60987375 | 62728589 | −0.0439 | −0.2847 | 0.8647 | 0.7772 | 1 | 1 |
| WCM0_T5 | 2 | 62729584 | 69784085 | 0.3579 | 0.1258 | 1.1111 | 1.0711 | 1 | 1 |
| WCM0_T5 | 2 | 69870178 | 70692791 | −0.916 | −1.1869 | NA | NA | NA | NA |
| WCM0_T5 | 2 | 70890857 | 72562126 | −0.4427 | −0.6949 | 1.2279 | 0.0077 | 1 | 0 |
| WCM0_T5 | 2 | 72606813 | 74901675 | −0.7292 | −0.9919 | 1.0087 | −0.0031 | 1 | 0 |
| WCM0_T5 | 2 | 74901986 | 85051326 | −0.4851 | −0.7387 | 1.1997 | −0.0012 | 1 | 0 |
| WCM0_T5 | 2 | 85059173 | 91955308 | −0.8325 | −1.0996 | 0.9391 | −0.0058 | 1 | 0 |
| WCM0_T5 | 2 | 95537509 | 101619146 | 0.2918 | 0.0585 | 1.0637 | 1.0191 | 1 | 1 |
| WCM0_T5 | 2 | 101620779 | 102493554 | −0.1739 | −0.418 | NA | NA | NA | NA |
| WCM0_T5 | 2 | 102499135 | 109003812 | 0.1811 | −0.0544 | 1.2629 | 0.663 | 1 | 1 |
| WCM0_T5 | 2 | 109065678 | 113958972 | −0.057 | −0.2981 | 1.0559 | 0.5707 | 1 | 1 |
| WCM0_T5 | 2 | 113976088 | 116066882 | −0.5524 | −0.8083 | NA | NA | NA | NA |
| WCM0_T5 | 2 | 116101464 | 116594317 | 0.0924 | −0.1451 | NA | NA | NA | NA |
| WCM0_T5 | 2 | 116598398 | 120690161 | 0.3209 | 0.0882 | 1.1183 | 1.0078 | 1 | 1 |
| WCM0_T5 | 2 | 120692564 | 127956991 | −0.6041 | −0.862 | 1.1014 | −0.001 | 1 | 0 |
| WCM0_T5 | 2 | 127958890 | 136872586 | −0.7322 | −0.995 | 1.0082 | −0.0047 | 1 | 0 |
| WCM0_T5 | 2 | 136872794 | 149522514 | −0.4698 | −0.7229 | 1.2071 | 0.0046 | 1 | 0 |
| WCM0_T5 | 2 | 149526799 | 153415244 | 0.2954 | 0.0622 | 1.0526 | 1.0355 | 1 | 1 |
| WCM0_T5 | 2 | 153417524 | 159533201 | 0.5026 | 0.2731 | 1.2244 | 1.1924 | 1 | 1 |
| WCM0_T5 | 2 | 159535142 | 160824103 | 0.3223 | 0.0896 | 1.0966 | 1.0315 | 1 | 1 |
| WCM0_T5 | 2 | 160825892 | 170139346 | 0.5688 | 0.3404 | 1.2854 | 1.2468 | 1 | 1 |
| WCM0_T5 | 2 | 170145672 | 176844577 | 0.2852 | 0.0517 | 1.0629 | 1.0101 | 1 | 1 |
| WCM0_T5 | 2 | 176857114 | 179393418 | 0.4971 | 0.2675 | 1.2417 | 1.1658 | 1 | 1 |
| WCM0_T5 | 2 | 179393715 | 182780509 | 0.6357 | 0.4083 | 1.3339 | 1.3204 | 1 | 1 |
| WCM0_T5 | 2 | 182780864 | 193059282 | 0.4642 | 0.2341 | 1.1845 | 1.1678 | 1 | 1 |
| WCM0_T5 | 2 | 196544875 | 201535333 | 0.3324 | 0.0999 | 1.0826 | 1.0608 | 1 | 1 |
| WCM0_T5 | 2 | 201677926 | 204360039 | 0.0943 | −0.1432 | 0.9222 | 0.8888 | 1 | 1 |
| WCM0_T5 | 2 | 204571465 | 219082179 | 0.3887 | 0.1572 | 1.1367 | 1.0936 | 1 | 1 |
| WCM0_T5 | 2 | 219090625 | 219679694 | 0.0701 | −0.168 | 0.9101 | 0.8701 | 1 | 1 |
| WCM0_T5 | 2 | 219688582 | 223918019 | 0.3587 | 0.1267 | 1.1207 | 1.0629 | 1 | 1 |
| WCM0_T5 | 2 | 224462273 | 232458780 | −0.0803 | −0.3219 | 1.0133 | 0.5867 | 1 | 1 |
| WCM0_T5 | 2 | 232573386 | 234343503 | −0.7499 | −1.0135 | 0.9905 | 2.00E−04 | 1 | 0 |
| WCM0_T5 | 2 | 234344474 | 234638237 | 0.0522 | −0.1863 | 1.1118 | 0.646 | 1 | 1 |
| WCM0_T5 | 2 | 234652130 | 234835251 | 0.1903 | −0.0451 | NA | NA | NA | NA |
| WCM0_T5 | 2 | 234839350 | 242003488 | 0.4525 | 0.2222 | 1.1795 | 1.1534 | 1 | 1 |
| WCM0_T5 | 2 | 242004333 | 242815429 | 0.1952 | −0.0401 | 0.9915 | 0.9537 | 1 | 1 |
| WCM0_T5 | 20 | 68342 | 2361665 | 0.4375 | 0.2069 | 1.1624 | 1.146 | 1 | 1 |
| WCM0_T5 | 20 | 2375300 | 6017803 | 0.1498 | −0.0864 | 0.9544 | 0.9293 | 1 | 1 |
| WCM0_T5 | 20 | 6021844 | 25666786 | 0.4747 | 0.2448 | 1.2054 | 1.1644 | 1 | 1 |
| WCM0_T5 | 20 | 25667001 | 30556187 | 0.0192 | −0.22 | NA | NA | NA | NA |
| WCM0_T5 | 20 | 30556347 | 31893802 | 0.1717 | −0.0641 | 0.9996 | 0.9136 | 1 | 1 |
| WCM0_T5 | 20 | 31894708 | 35884833 | −0.018 | −0.2581 | 0.8371 | 0.8354 | 1 | 1 |
| WCM0_T5 | 20 | 35885245 | 45633591 | 0.3188 | 0.086 | 1.0647 | 1.0582 | 1 | 1 |
| WCM0_T5 | 20 | 45644934 | 50808209 | 0.0642 | −0.1739 | 0.9037 | 0.8692 | 1 | 1 |
| WCM0_T5 | 20 | 51589859 | 55213096 | 0.2249 | −0.0098 | NA | NA | NA | NA |
| WCM0_T5 | 20 | 55746137 | 60419867 | 0.5245 | 0.2954 | 1.2384 | 1.216 | 1 | 1 |
| WCM0_T5 | 20 | 60427944 | 62076075 | 0.3162 | 0.0833 | 1.0735 | 1.0454 | 1 | 1 |
| WCM0_T5 | 20 | 62076743 | 62700868 | 0.0642 | −0.174 | 0.8964 | 0.8764 | 1 | 1 |
| WCM0_T5 | 20 | 62701070 | 62904990 | 0.5196 | 0.2904 | NA | NA | NA | NA |
| WCM0_T5 | 21 | 10862618 | 32503230 | 0.4366 | 0.206 | 1.1617 | 1.1453 | 1 | 1 |
| WCM0_T5 | 21 | 32508359 | 36088748 | 0.148 | −0.0883 | 0.9685 | 0.9127 | 1 | 1 |
| WCM0_T5 | 21 | 36164460 | 37507706 | −0.6833 | −0.9442 | NA | NA | NA | NA |
| WCM0_T5 | 21 | 37510213 | 38612782 | 0.1007 | −0.1366 | 0.9348 | 0.8846 | 1 | 1 |
| WCM0_T5 | 21 | 38639544 | 44108030 | 0.4449 | 0.2144 | 1.1603 | 1.1603 | 1 | 1 |
| WCM0_T5 | 21 | 44117541 | 45859002 | 0.1988 | −0.0364 | 1.0119 | 0.9383 | 1 | 1 |
| WCM0_T5 | 21 | 45860766 | 48084273 | 0.4117 | 0.1806 | 1.1761 | 1.0907 | 1 | 1 |
| WCM0_T5 | 22 | 16255826 | 28315134 | −0.743 | −1.0063 | 1.0005 | −0.0049 | 1 | 0 |
| WCM0_T5 | 22 | 29092957 | 32644703 | −0.9024 | −1.1726 | 0.8937 | −0.0065 | 1 | 0 |
| WCM0_T5 | 22 | 32647780 | 37966641 | −0.7222 | −0.9846 | 1.0154 | −0.0046 | 1 | 0 |
| WCM0_T5 | 22 | 37967820 | 39111897 | −0.9942 | −1.2688 | 0.8408 | −0.0108 | 1 | 0 |
| WCM0_T5 | 22 | 39112118 | 41075695 | −0.8442 | −1.1118 | 0.9294 | −0.004 | 1 | 0 |
| WCM0_T5 | 22 | 41077028 | 43442599 | −1.0486 | −1.3259 | 0.809 | −0.0112 | 1 | 0 |
| WCM0_T5 | 22 | 43447870 | 51216348 | −0.7632 | −1.0273 | 0.9863 | −0.0051 | 1 | 0 |
| WCM0_T5 | 3 | 361508 | 9422287 | −0.4799 | −0.7334 | 1.2035 | −5.00E−04 | 1 | 0 |
| WCM0_T5 | 3 | 9424993 | 10357118 | −0.9042 | −1.1745 | 0.8875 | −0.0014 | 1 | 0 |
| WCM0_T5 | 3 | 10359800 | 31789625 | −0.5516 | −0.8076 | 1.1453 | −0.0027 | 1 | 0 |
| WCM0_T5 | 3 | 31871687 | 33731348 | −0.8302 | −1.0972 | NA | NA | NA | NA |
| WCM0_T5 | 3 | 33738372 | 37458982 | −0.5982 | −0.8558 | 1.102 | 0.0031 | 1 | 0 |
| WCM0_T5 | 3 | 37476427 | 40286017 | −0.4444 | −0.6966 | 1.2353 | −0.0013 | 1 | 0 |
| WCM0_T5 | 3 | 40291743 | 47050774 | −0.6016 | −0.8594 | 1.0999 | 0.0025 | 1 | 0 |
| WCM0_T5 | 3 | 47058783 | 50214261 | −0.954 | −1.2267 | 0.8647 | −0.0101 | 1 | 0 |
| WCM0_T5 | 3 | 50219843 | 53381422 | −0.686 | −0.947 | 1.0465 | −0.0091 | 1 | 0 |
| WCM0_T5 | 3 | 53529240 | 56468916 | −0.3997 | −0.6505 | 1.2682 | 0.0059 | 1 | 0 |
| WCM0_T5 | 3 | 56598022 | 58555562 | −0.8458 | −1.1135 | 0.9327 | −0.0083 | 1 | 0 |
| WCM0_T5 | 3 | 58620028 | 68802060 | −0.4303 | −0.6821 | 1.2444 | 0.0021 | 1 | 0 |
| WCM0_T5 | 3 | 68929871 | 73657708 | −0.6472 | −0.9067 | 1.0579 | 0.0089 | 1 | 0 |
| WCM0_T5 | 3 | 73673435 | 93761897 | 0.1302 | −0.1065 | 1.207 | 0.6507 | 1 | 1 |
| WCM0_T5 | 3 | 93768315 | 113114614 | −0.4864 | −0.74 | 1.1993 | −0.0018 | 1 | 0 |
| WCM0_T5 | 3 | 113115489 | 121642048 | −0.6339 | −0.8929 | 1.0751 | 0.0019 | 1 | 0 |
| WCM0_T5 | 3 | 121642117 | 122103099 | −0.5579 | −0.8141 | NA | NA | NA | NA |
| WCM0_T5 | 3 | 122121725 | 122405951 | 0.2513 | 0.0172 | NA | NA | NA | NA |
| WCM0_T5 | 3 | 122411180 | 124281962 | 0.4722 | 0.2422 | 1.1852 | 1.1804 | 1 | 1 |
| WCM0_T5 | 3 | 124303724 | 125690994 | 0.2641 | 0.0303 | 1.0389 | 1.0035 | 1 | 1 |
| WCM0_T5 | 3 | 125694535 | 128205878 | 0.5664 | 0.338 | 1.2986 | 1.2293 | 1 | 1 |
| WCM0_T5 | 3 | 128292142 | 129696079 | 0.1643 | −0.0716 | 0.9661 | 0.937 | 1 | 1 |
| WCM0_T5 | 3 | 130159196 | 134967282 | 0.4507 | 0.2203 | 1.1994 | 1.1306 | 1 | 1 |
| WCM0_T5 | 3 | 134968251 | 138664459 | 0.1866 | −0.0488 | NA | NA | NA | NA |
| WCM0_T5 | 3 | 138664743 | 141231124 | 0.5223 | 0.2931 | NA | NA | NA | NA |
| WCM0_T5 | 3 | 141235207 | 142606548 | 0.1129 | −0.1241 | 0.9448 | 0.8904 | 1 | 1 |
| WCM0_T5 | 3 | 142607706 | 169485419 | 0.4456 | 0.2152 | 1.1667 | 1.1549 | 1 | 1 |
| WCM0_T5 | 3 | 169485469 | 170198063 | 0.0992 | −0.1382 | 0.9347 | 0.8827 | 1 | 1 |
| WCM0_T5 | 3 | 170198537 | 182563224 | 0.3312 | 0.0986 | 1.086 | 1.0555 | 1 | 1 |
| WCM0_T5 | 3 | 182566270 | 186760657 | 0.1093 | −0.1278 | 0.9358 | 0.8946 | 1 | 1 |
| WCM0_T5 | 3 | 186760715 | 194081532 | 0.4665 | 0.2364 | 1.2092 | 1.1469 | 1 | 1 |
| WCM0_T5 | 3 | 194081784 | 197847354 | 0.0568 | −0.1816 | 0.8908 | 0.8727 | 1 | 1 |
| WCM0_T5 | 4 | 53435 | 338171 | 0.1375 | −0.099 | NA | NA | NA | NA |
| WCM0_T5 | 4 | 343826 | 3219663 | −0.2166 | −0.4619 | 0.9694 | 0.4827 | 1 | 0 |
| WCM0_T5 | 4 | 3222036 | 10027542 | 0.0051 | −0.2345 | 1.1303 | 0.5697 | 1 | 1 |
| WCM0_T5 | 4 | 10077008 | 25032141 | 0.4354 | 0.2047 | 1.1619 | 1.1431 | 1 | 1 |
| WCM0_T5 | 4 | 25125613 | 26432569 | 0.1237 | −0.1131 | NA | NA | NA | NA |
| WCM0_T5 | 4 | 26483424 | 38682329 | 0.4453 | 0.2148 | 1.1912 | 1.1298 | 1 | 1 |
| WCM0_T5 | 4 | 38690410 | 44631587 | −0.4687 | −0.7218 | 0.9254 | 0.2873 | 1 | 0 |
| WCM0_T5 | 4 | 44637960 | 56236295 | 0.3753 | 0.1436 | 1.1373 | 1.072 | 1 | 1 |
| WCM0_T5 | 4 | 56262374 | 57976100 | 0.1379 | −0.0986 | 0.9568 | 0.911 | 1 | 1 |
| WCM0_T5 | 4 | 57976319 | 71467233 | 0.4873 | 0.2576 | 1.2174 | 1.1736 | 1 | 1 |
| WCM0_T5 | 4 | 71467386 | 71895049 | 0.1161 | −0.1209 | NA | NA | NA | NA |
| WCM0_T5 | 4 | 72102276 | 73615956 | −0.4526 | −0.7051 | NA | NA | NA | NA |
| WCM0_T5 | 4 | 73923990 | 75971359 | 0.1092 | −0.128 | NA | NA | NA | NA |
| WCM0_T5 | 4 | 76407746 | 77357362 | −0.2223 | −0.4678 | 0.929 | 0.5171 | 1 | 1 |
| WCM0_T5 | 4 | 77476790 | 84406140 | 0.0901 | −0.1475 | 1.153 | 0.6526 | 1 | 1 |
| WCM0_T5 | 4 | 84457953 | 85654760 | 0.3795 | 0.1478 | NA | NA | NA | NA |
| WCM0_T5 | 4 | 85656183 | 89573149 | 0.2514 | 0.0173 | 1.0296 | 0.9945 | 1 | 1 |
| WCM0_T5 | 4 | 89574099 | 103947745 | 0.4441 | 0.2136 | 1.1773 | 1.1419 | 1 | 1 |
| WCM0_T5 | 4 | 103949924 | 106158602 | 0.0089 | −0.2306 | NA | NA | NA | NA |
| WCM0_T5 | 4 | 106290846 | 106473981 | −1.3806 | −1.6771 | NA | NA | NA | NA |
| WCM0_T5 | 4 | 106510518 | 177072944 | 0.0073 | −0.2322 | 1.1207 | 0.582 | 1 | 1 |
| WCM0_T5 | 4 | 177077262 | 178911649 | −1.3509 | −1.6455 | NA | NA | NA | NA |
| WCM0_T5 | 4 | 183090470 | 190947544 | −0.1117 | −0.3541 | 1.0079 | 0.5568 | 1 | 1 |
| WCM0_T5 | 5 | 140480 | 1494918 | 0.4271 | 0.1963 | 1.1761 | 1.1155 | 1 | 1 |
| WCM0_T5 | 5 | 1501627 | 9629803 | 0.6225 | 0.395 | 1.347 | 1.2829 | 1 | 1 |
| WCM0_T5 | 5 | 9629973 | 31541142 | 0.4235 | 0.1927 | 1.1603 | 1.1254 | 1 | 1 |
| WCM0_T5 | 5 | 31541484 | 32591723 | −0.0245 | −0.2647 | NA | NA | NA | NA |
| WCM0_T5 | 5 | 32599039 | 36680713 | 0.4316 | 0.2009 | 1.1857 | 1.1132 | 1 | 1 |
| WCM0_T5 | 5 | 36684128 | 37605256 | 0.1121 | −0.125 | NA | NA | NA | NA |
| WCM0_T5 | 5 | 37697813 | 54555656 | 0.4576 | 0.2273 | 1.1933 | 1.148 | 1 | 1 |
| WCM0_T5 | 5 | 54557146 | 56558618 | 0.1665 | −0.0694 | NA | NA | NA | NA |
| WCM0_T5 | 5 | 56777447 | 57755813 | 0.3248 | 0.0921 | NA | NA | NA | NA |
| WCM0_T5 | 5 | 57790532 | 60999779 | 0.0791 | −0.1588 | NA | NA | NA | NA |
| WCM0_T5 | 5 | 61602360 | 68399889 | −0.0716 | −0.313 | 1.0683 | 0.5417 | 1 | 1 |
| WCM0_T5 | 5 | 68400471 | 70754627 | −0.5138 | −0.7684 | NA | NA | NA | NA |
| WCM0_T5 | 5 | 70759893 | 70952634 | −0.0792 | −0.3209 | NA | NA | NA | NA |
| WCM0_T5 | 5 | 71015196 | 74014140 | 0.0874 | −0.1503 | 1.1775 | 0.6246 | 1 | 1 |
| WCM0_T5 | 5 | 74014644 | 80911327 | −0.169 | −0.413 | 0.9756 | 0.5266 | 1 | 1 |
| WCM0_T5 | 5 | 80932351 | 122950093 | 0.0677 | −0.1704 | 1.1813 | 0.5959 | 1 | 1 |
| WCM0_T5 | 5 | 123966411 | 129072749 | −1.4002 | −1.698 | 0.6076 | 0.0088 | 1 | 0 |
| WCM0_T5 | 5 | 129240668 | 134347241 | −0.744 | −1.0074 | 0.9998 | −0.0049 | 1 | 0 |
| WCM0_T5 | 5 | 134364442 | 136957844 | −0.3017 | −0.5495 | 1.3658 | 7.00E−04 | 1 | 0 |
| WCM0_T5 | 5 | 136961468 | 139189010 | 0.0012 | −0.2384 | 0.8989 | 0.7965 | 1 | 1 |
| WCM0_T5 | 5 | 139189310 | 140174952 | 0.2075 | −0.0275 | 1.0011 | 0.9611 | 1 | 1 |
| WCM0_T5 | 5 | 140175184 | 146080618 | 0.3725 | 0.1407 | 1.1058 | 1.0991 | 1 | 1 |
| WCM0_T5 | 5 | 146080833 | 148695769 | 0.5886 | 0.3606 | 1.3009 | 1.267 | 1 | 1 |
| WCM0_T5 | 5 | 148697279 | 170814964 | 0.3541 | 0.122 | 1.0915 | 1.0849 | 1 | 1 |
| WCM0_T5 | 5 | 170817086 | 180687730 | 0.1558 | −0.0803 | 0.9546 | 0.9372 | 1 | 1 |
| WCM0_T5 | 6 | 292587 | 6654874 | 0.3923 | 0.1609 | 1.1282 | 1.1077 | 1 | 1 |
| WCM0_T5 | 6 | 7182266 | 16328478 | 0.2823 | 0.0488 | 1.0555 | 1.0133 | 1 | 1 |
| WCM0_T5 | 6 | 17283149 | 18258553 | −0.0033 | −0.2431 | NA | NA | NA | NA |
| WCM0_T5 | 6 | 18264068 | 30314192 | 0.3373 | 0.1048 | 1.083 | 1.0677 | 1 | 1 |
| WCM0_T5 | 6 | 30314598 | 31948278 | 0.1321 | −0.1045 | 0.9368 | 0.9235 | 1 | 1 |
| WCM0_T5 | 6 | 32006886 | 34215221 | 0.2571 | 0.0231 | 1.0348 | 0.9975 | 1 | 1 |
| WCM0_T5 | 6 | 34256548 | 37438776 | 0.1004 | −0.1369 | 0.9188 | 0.9002 | 1 | 1 |
| WCM0_T5 | 6 | 37439670 | 41710228 | 0.434 | 0.2034 | 1.1783 | 1.1244 | 1 | 1 |
| WCM0_T5 | 6 | 41710989 | 43742111 | 0.0285 | −0.2105 | 0.8669 | 0.8616 | 1 | 1 |
| WCM0_T5 | 6 | 43745354 | 62995719 | 0.4677 | 0.2376 | 1.1874 | 1.1706 | 1 | 1 |
| WCM0_T5 | 6 | 63990020 | 72970416 | 0.072 | −0.166 | 1.1785 | 0.6041 | 1 | 1 |
| WCM0_T5 | 6 | 72974683 | 74516792 | −0.3582 | −0.6077 | NA | NA | NA | NA |
| WCM0_T5 | 6 | 74517837 | 86199223 | 0.0205 | −0.2187 | 1.0934 | 0.6253 | 1 | 1 |
| WCM0_T5 | 6 | 86200393 | 90605580 | −0.2047 | −0.4497 | NA | NA | NA | NA |
| WCM0_T5 | 6 | 90605688 | 102511911 | 0.1094 | −0.1278 | 1.2006 | 0.6299 | 1 | 1 |
| WCM0_T5 | 6 | 102516262 | 109699187 | −0.1445 | −0.3878 | 1.0428 | 0.4857 | 1 | 0 |
| WCM0_T5 | 6 | 109700823 | 110759943 | 0.0382 | −0.2006 | 1.1347 | 0.6057 | 1 | 1 |
| WCM0_T5 | 6 | 110760088 | 111912850 | −0.28 | −0.5272 | NA | NA | NA | NA |
| WCM0_T5 | 6 | 111913064 | 131190710 | 0.1183 | −0.1187 | 1.2308 | 0.6113 | 1 | 1 |
| WCM0_T5 | 6 | 131191147 | 139694553 | −0.0404 | −0.281 | 1.1283 | 0.5177 | 1 | 1 |
| WCM0_T5 | 6 | 139694845 | 149395201 | 0.1163 | −0.1206 | 1.2206 | 0.6189 | 1 | 1 |
| WCM0_T5 | 6 | 149691115 | 152201804 | −0.3078 | −0.5558 | 0.9108 | 0.4498 | 1 | 0 |
| WCM0_T5 | 6 | 152265330 | 159465156 | −0.0773 | −0.3188 | 1.0697 | 0.5337 | 1 | 1 |
| WCM0_T5 | 6 | 159590665 | 170893364 | 0.0827 | −0.155 | 1.1985 | 0.5978 | 1 | 1 |
| WCM0_T5 | 7 | 193546 | 4854888 | 0.1376 | −0.099 | 0.9423 | 0.9251 | 1 | 1 |
| WCM0_T5 | 7 | 4855804 | 7118635 | −0.1542 | −0.3978 | 0.7793 | 0.7387 | 1 | 1 |
| WCM0_T5 | 7 | 7274011 | 23015831 | 0.4973 | 0.2678 | 1.2162 | 1.1917 | 1 | 1 |
| WCM0_T5 | 7 | 23016406 | 24331250 | 0.1181 | −0.1188 | NA | NA | NA | NA |
| WCM0_T5 | 7 | 24663462 | 27196069 | 0.4498 | 0.2194 | 1.1962 | 1.1323 | 1 | 1 |
| WCM0_T5 | 7 | 27203312 | 27689281 | −0.4206 | −0.6721 | NA | NA | NA | NA |
| WCM0_T5 | 7 | 27702390 | 29160637 | 0.4446 | 0.2142 | NA | NA | NA | NA |
| WCM0_T5 | 7 | 29394275 | 38398363 | −0.4683 | −0.7213 | 1.2147 | −0.0016 | 1 | 0 |
| WCM0_T5 | 7 | 38402603 | 39504327 | 0.5472 | 0.3184 | NA | NA | NA | NA |
| WCM0_T5 | 7 | 39606016 | 48035664 | 0.2831 | 0.0496 | 1.0466 | 1.0234 | 1 | 1 |
| WCM0_T5 | 7 | 48045605 | 55565396 | 0.5439 | 0.3151 | 1.2744 | 1.2137 | 1 | 1 |
| WCM0_T5 | 7 | 55588758 | 77584157 | −0.0109 | −0.2508 | 0.9213 | 0.7595 | 1 | 1 |
| WCM0_T5 | 7 | 77648671 | 89793914 | 0.662 | 0.4351 | 1.3877 | 1.3163 | 1 | 1 |
| WCM0_T5 | 7 | 89794026 | 92164223 | 0.3163 | 0.0835 | 1.0693 | 1.0498 | 1 | 1 |
| WCM0_T5 | 7 | 92166211 | 97365628 | 0.7269 | 0.501 | 1.4596 | 1.3706 | 1 | 1 |
| WCM0_T5 | 7 | 97369163 | 99769838 | 0.216 | −0.0188 | 1.0837 | 0.8903 | 1 | 1 |
| WCM0_T5 | 7 | 99771618 | 102098129 | 0.0647 | −0.1735 | 1.0409 | 0.7325 | 1 | 1 |
| WCM0_T5 | 7 | 102098330 | 102584920 | 0.2001 | −0.035 | NA | NA | NA | NA |
| WCM0_T5 | 7 | 102604130 | 107704419 | 0.4795 | 0.2496 | 1.3328 | 1.045 | 1 | 1 |
| WCM0_T5 | 7 | 107706389 | 127847944 | 0.6966 | 0.4702 | 1.5526 | 1.218 | 2 | 1 |
| WCM0_T5 | 7 | 127892259 | 129921926 | 0.2838 | 0.0504 | 1.157 | 0.9141 | 1 | 1 |
| WCM0_T5 | 7 | 129929506 | 137761334 | 0.5697 | 0.3413 | 1.4034 | 1.1305 | 1 | 1 |
| WCM0_T5 | 7 | 137773291 | 140549976 | 0.2426 | 0.0083 | 1.1238 | 0.8877 | 1 | 1 |
| WCM0_T5 | 7 | 140624314 | 147600604 | 0.7231 | 0.4971 | 1.6204 | 1.2023 | 2 | 1 |
| WCM0_T5 | 7 | 147675028 | 149152447 | 0.2201 | −0.0147 | 1.1141 | 0.8656 | 1 | 1 |
| WCM0_T5 | 7 | 149152770 | 150940797 | 0.6194 | 0.3918 | 1.4466 | 1.1775 | 1 | 1 |
| WCM0_T5 | 7 | 150945527 | 158937416 | 0.44 | 0.2094 | 1.2092 | 1.1033 | 1 | 1 |
| WCM0_T5 | 8 | 192877 | 2092748 | −4.7905 | −6.5151 | NA | NA | NA | NA |
| WCM0_T5 | 8 | 2796243 | 36703376 | −0.6447 | −0.9041 | 1.0725 | −0.0038 | 1 | 0 |
| WCM0_T5 | 8 | 36721946 | 37553655 | 0.5943 | 0.3664 | NA | NA | NA | NA |
| WCM0_T5 | 8 | 37553725 | 38458696 | 0.0157 | −0.2236 | NA | NA | NA | NA |
| WCM0_T5 | 8 | 38614885 | 41800506 | 0.3129 | 0.08 | 1.0839 | 1.03 | 1 | 1 |
| WCM0_T5 | 8 | 41801451 | 48889235 | 0.0475 | −0.1911 | NA | NA | NA | NA |
| WCM0_T5 | 8 | 48921068 | 67342143 | 0.4449 | 0.2145 | 1.1721 | 1.1485 | 1 | 1 |
| WCM0_T5 | 8 | 67342380 | 68421839 | 0.0749 | −0.163 | NA | NA | NA | NA |
| WCM0_T5 | 8 | 68423846 | 95186078 | 0.4403 | 0.2098 | 1.1847 | 1.1282 | 1 | 1 |
| WCM0_T5 | 8 | 95186439 | 104078707 | 0.227 | −0.0076 | 1.0079 | 0.9815 | 1 | 1 |
| WCM0_T5 | 8 | 104080960 | 105601083 | 0.4751 | 0.2451 | NA | NA | NA | NA |
| WCM0_T5 | 8 | 106430808 | 120575116 | −0.4138 | −0.6651 | 1.2544 | 0.0069 | 1 | 0 |
| WCM0_T5 | 8 | 120577219 | 131249217 | −0.6932 | −0.9545 | 1.0347 | −0.0027 | 1 | 0 |
| WCM0_T5 | 8 | 131370406 | 141262869 | −0.374 | −0.624 | 1.2914 | 0.0064 | 1 | 0 |
| WCM0_T5 | 8 | 141285752 | 142229001 | −0.719 | −0.9813 | 1.0229 | −0.0099 | 1 | 0 |
| WCM0_T5 | 8 | 142229145 | 143922636 | −0.4404 | −0.6925 | 1.2414 | −0.0038 | 1 | 0 |
| WCM0_T5 | 8 | 143927973 | 146032544 | −0.7631 | −1.0272 | 0.9849 | −0.0035 | 1 | 0 |
| WCM0_T5 | 8 | 146032848 | 146279384 | −0.4727 | −0.726 | NA | NA | NA | NA |
| WCM0_T5 | 9 | 117666 | 3395422 | −1.3193 | −1.612 | 0.6594 | −0.0051 | 1 | 0 |
| WCM0_T5 | 9 | 3828205 | 19032907 | 0.0736 | −0.1643 | 1.1425 | 0.6423 | 1 | 1 |
| WCM0_T5 | 9 | 19049645 | 19550205 | −0.7812 | −1.0461 | NA | NA | NA | NA |
| WCM0_T5 | 9 | 19573373 | 32973499 | −0.4051 | −0.6561 | 1.2745 | −0.0053 | 1 | 0 |
| WCM0_T5 | 9 | 32973702 | 34500884 | −0.8129 | −1.0792 | NA | NA | NA | NA |
| WCM0_T5 | 9 | 34501161 | 35870225 | −0.5719 | −0.8286 | 1.1325 | −0.0064 | 1 | 0 |
| WCM0_T5 | 9 | 35906276 | 40772487 | −0.7546 | −1.0183 | NA | NA | NA | NA |
| WCM0_T5 | 9 | 40773337 | 99417031 | −0.0483 | −0.2892 | 1.0549 | 0.5818 | 1 | 1 |
| WCM0_T5 | 9 | 99521250 | 113704435 | 0.3778 | 0.1461 | 1.1144 | 1.0987 | 1 | 1 |
| WCM0_T5 | 9 | 113734307 | 116263915 | 0.1303 | −0.1063 | 0.9413 | 0.9166 | 1 | 1 |
| WCM0_T5 | 9 | 116267817 | 116303604 | 0.3412 | 0.1089 | NA | NA | NA | NA |
| WCM0_T5 | 9 | 116345866 | 123234032 | 0.5368 | 0.3078 | 1.2555 | 1.2202 | 1 | 1 |
| WCM0_T5 | 9 | 123239687 | 127572700 | 0.3013 | 0.0681 | 1.0516 | 1.0452 | 1 | 1 |
| WCM0_T5 | 9 | 127615904 | 128124840 | −0.0981 | −0.3402 | NA | NA | NA | NA |
| WCM0_T5 | 9 | 128201226 | 130279371 | 0.2496 | 0.0154 | 1.0365 | 0.985 | 1 | 1 |
| WCM0_T5 | 9 | 130280142 | 131115846 | 0.0185 | −0.2207 | 0.8713 | 0.845 | 1 | 1 |
| WCM0_T5 | 9 | 131117342 | 131830527 | −0.1559 | −0.3996 | 0.7721 | 0.7441 | 1 | 1 |
| WCM0_T5 | 9 | 131831378 | 134404542 | 0.0387 | −0.2001 | 0.8857 | 0.8554 | 1 | 1 |
| WCM0_T5 | 9 | 134404866 | 139694251 | 0.2737 | 0.04 | 1.048 | 1.0082 | 1 | 1 |
| WCM0_T5 | 9 | 139694603 | 140695352 | 0.0972 | −0.1402 | 0.9213 | 0.8935 | 1 | 1 |
| WCM0_T5 | 9 | 140705966 | 141071438 | 0.4393 | 0.2088 | NA | NA | NA | NA |
| WCM0_T5 | X | 2700147 | 16608982 | 0.3254 | 0.0927 | NA | NA | NA | NA |
| WCM0_T5 | X | 16627816 | 24906100 | 0.1207 | −0.1162 | NA | NA | NA | NA |
| WCM0_T5 | X | 24948585 | 39934425 | 0.4371 | 0.2065 | NA | NA | NA | NA |
| WCM0_T5 | X | 39935760 | 49595653 | 0.0794 | −0.1584 | NA | NA | NA | NA |
| WCM0_T5 | X | 49597268 | 69898729 | 0.3739 | 0.1421 | NA | NA | NA | NA |
| WCM0_T5 | X | 69902707 | 70832812 | −0.0075 | −0.2474 | NA | NA | NA | NA |
| WCM0_T5 | X | 70836204 | 99943341 | 0.3898 | 0.1583 | NA | NA | NA | NA |
| WCM0_T5 | X | 99944845 | 100662698 | 0.0673 | −0.1708 | NA | NA | NA | NA |
| WCM0_T5 | X | 100808315 | 112058643 | 0.5208 | 0.2917 | NA | NA | NA | NA |
| WCM0_T5 | X | 113965735 | 129765504 | 0.1886 | −0.0468 | NA | NA | NA | NA |
| WCM0_T5 | X | 129768990 | 132092590 | 0.597 | 0.3691 | NA | NA | NA | NA |
| WCM0_T5 | X | 132159668 | 139047663 | 0.3335 | 0.1009 | NA | NA | NA | NA |
| WCM0_T5 | X | 139585986 | 152845607 | 0.6167 | 0.3891 | NA | NA | NA | NA |
| WCM0_T5 | X | 152913476 | 154509348 | 0.298 | 0.0648 | NA | NA | NA | NA |
| WCM1 | 1 | 14907 | 193181531 | −0.0854 | −0.3418 | 0.7918 | 0.7863 | 1 | 1 |
| WCM1 | 1 | 193202255 | 248569369 | 0.0677 | 0.1306 | 1.0947 | 1.0947 | 1 | 1 |
| WCM1 | 1 | 248569587 | 249213973 | −0.1907 | −0.7444 | 0.6781 | 0.5157 | 1 | 1 |
| WCM1 | 10 | 226104 | 135477928 | −0.0735 | −0.3011 | 0.8144 | 0.8089 | 1 | 1 |
| WCM1 | 11 | 193127 | 62201391 | 0.085 | 0.1779 | 1.1558 | 1.1067 | 1 | 1 |
| WCM1 | 11 | 62259275 | 68675803 | −0.0578 | −0.2484 | 0.8418 | 0.8418 | 1 | 1 |
| WCM1 | 11 | 68676119 | 134605954 | 0.081 | 0.1671 | 1.1228 | 1.1228 | 1 | 1 |
| WCM1 | 12 | 148452 | 94543591 | −0.0792 | −0.3206 | 0.8227 | 0.7788 | 1 | 1 |
| WCM1 | 12 | 94563000 | 118610483 | 0.2443 | 0.5753 | 1.5346 | 1.4454 | 2 | 1 |
| WCM1 | 12 | 118614980 | 133810868 | −0.2304 | −0.9214 | 1.0465 | 0.0095 | 1 | 0 |
| WCM1 | 13 | 19041937 | 115089411 | 0.0416 | 0.057 | 1.0403 | 1.0403 | 1 | 1 |
| WCM1 | 14 | 19110401 | 107283270 | 0.0461 | 0.0698 | 1.0496 | 1.0496 | 1 | 1 |
| WCM1 | 15 | 20083910 | 102514170 | −0.0845 | −0.3389 | 0.7906 | 0.7906 | 1 | 1 |
| WCM1 | 16 | 64424 | 66511466 | 0.0423 | 0.0588 | 1.0416 | 1.0416 | 1 | 1 |
| WCM1 | 16 | 66514692 | 90067176 | −0.0891 | −0.3547 | 0.8229 | 0.7412 | 1 | 1 |
| WCM1 | 16 | 90071375 | 90168842 | −0.0027 | −0.0739 | 1.4705 | 0.4296 | 1 | 0 |
| WCM1 | 17 | 131595 | 18875062 | −0.285 | −1.1966 | 0.9356 | −0.063 | 1 | 0 |
| WCM1 | 17 | 18880268 | 81187850 | 0.0549 | 0.0946 | 1.0678 | 1.0678 | 1 | 1 |
| WCM1 | 18 | 47809 | 78005205 | 0.034 | 0.0351 | 1.0247 | 1.0247 | 1 | 1 |
| WCM1 | 19 | 105021 | 6496090 | −0.2073 | −0.8166 | 0.9918 | 0.1438 | 1 | 0 |
| WCM1 | 19 | 6496259 | 7929854 | 0.0968 | 0.2098 | 1.1782 | 1.1348 | 1 | 1 |
| WCM1 | 19 | 7933996 | 10979917 | −0.2663 | −1.0976 | 0.9731 | −0.0385 | 1 | 0 |
| WCM1 | 19 | 11015622 | 12164014 | 0.286 | 0.6697 | 1.7391 | 1.4424 | 2 | 1 |
| WCM1 | 19 | 12167107 | 14259802 | −0.0986 | −0.3882 | 0.8371 | 0.6911 | 1 | 1 |
| WCM1 | 19 | 14260473 | 16033216 | 0.5175 | 1.141 | 2.9309 | 1.4797 | 3 | 1 |
| WCM1 | 19 | 16033280 | 32844780 | −0.195 | −0.7632 | 0.9789 | 0.1995 | 1 | 0 |
| WCM1 | 19 | 32845102 | 35250928 | 0.1442 | 0.3333 | 1.3687 | 1.151 | 1 | 1 |
| WCM1 | 19 | 35250969 | 36113853 | 0.5534 | 1.2078 | 3.3131 | 1.3066 | 3 | 1 |
| WCM1 | 19 | 36120222 | 36387315 | 0.1139 | 0.255 | 1.2655 | 1.1213 | 1 | 1 |
| WCM1 | 19 | 36387717 | 36590554 | −0.0848 | −0.3397 | NA | NA | NA | NA |
| WCM1 | 19 | 36591784 | 49547448 | −0.2711 | −1.1228 | 0.9633 | −0.0449 | 1 | 0 |
| WCM1 | 19 | 49552028 | 59093645 | 0.0055 | −0.049 | 0.9666 | 0.9666 | 1 | 1 |
| WCM1 | 2 | 41613 | 272203 | −0.0222 | −0.1341 | NA | NA | NA | NA |
| WCM1 | 2 | 272346 | 48706586 | −0.0933 | −0.3693 | 0.7742 | 0.7742 | 1 | 1 |
| WCM1 | 2 | 48707026 | 119750009 | 0.0191 | −0.0086 | 0.994 | 0.994 | 1 | 1 |
| WCM1 | 2 | 119750148 | 125627424 | −0.2428 | −0.9807 | 0.9278 | 0.0857 | 1 | 0 |
| WCM1 | 2 | 125660583 | 243037116 | 0.0487 | 0.0772 | 1.055 | 1.055 | 1 | 1 |
| WCM1 | 20 | 68423 | 62934541 | 0.0774 | 0.1572 | 1.1151 | 1.1151 | 1 | 1 |
| WCM1 | 21 | 9590370 | 48084149 | 0.037 | 0.0436 | 1.0307 | 1.0307 | 1 | 1 |
| WCM1 | 22 | 16158973 | 51223730 | 0.054 | 0.092 | 1.0659 | 1.0659 | 1 | 1 |
| WCM1 | 3 | 239314 | 197896800 | 0.0598 | 0.1084 | 1.0781 | 1.0781 | 1 | 1 |
| WCM1 | 4 | 53364 | 134481894 | −0.2566 | −1.0482 | 0.9928 | −0.0256 | 1 | 0 |
| WCM1 | 4 | 134629278 | 190882003 | 0.0343 | 0.0358 | 1.0251 | 1.0251 | 1 | 1 |
| WCM1 | 5 | 90680 | 139422102 | 0.0331 | 0.0325 | 1.0228 | 1.0228 | 1 | 1 |
| WCM1 | 5 | 139422215 | 141020563 | −0.1658 | −0.6416 | 0.6926 | 0.5893 | 1 | 1 |
| WCM1 | 5 | 141021110 | 180687814 | 0.0766 | 0.155 | 1.1134 | 1.1134 | 1 | 1 |
| WCM1 | 6 | 203424 | 25983541 | −0.1281 | −0.4953 | 0.7412 | 0.6776 | 1 | 1 |
| WCM1 | 6 | 25983736 | 33553364 | −0.2958 | −1.2567 | 0.9518 | −0.1148 | 1 | 0 |
| WCM1 | 6 | 33554557 | 170892848 | −0.0696 | −0.2879 | 0.8191 | 0.8191 | 1 | 1 |
| WCM1 | 7 | 193222 | 44578836 | 0.0808 | 0.1666 | 1.1224 | 1.1224 | 1 | 1 |
| WCM1 | 7 | 44579159 | 141720790 | −0.0124 | −0.1035 | 0.9537 | 0.9078 | 1 | 1 |
| WCM1 | 7 | 141721479 | 158937185 | 0.0998 | 0.2177 | 1.4546 | 0.8711 | 1 | 1 |
| WCM1 | 8 | 163470 | 42857261 | −0.2244 | −0.8938 | 1.0638 | 0.0126 | 1 | 0 |
| WCM1 | 8 | 42862972 | 146203412 | 0.475 | 1.0601 | 2.9306 | 1.2395 | 3 | 1 |
| WCM1 | 8 | 146203928 | 146279498 | −0.2444 | −0.9881 | NA | NA | NA | NA |
| WCM1 | 9 | 14879 | 141107545 | 0.0662 | 0.1263 | 1.0915 | 1.0915 | 1 | 1 |
| WCM1 | X | 2700202 | 154493576 | 0.0449 | 0.0663 | NA | NA | NA | NA |
| WCM10 | 1 | 14907 | 249213973 | 0.0209 | 0.1125 | 1.0811 | 1.0811 | 1 | 1 |
| WCM10 | 10 | 225920 | 135477891 | 0.0117 | 0.0637 | 1.0452 | 1.0452 | 1 | 1 |
| WCM10 | 11 | 193096 | 62303516 | 0.0559 | 0.2865 | 1.2197 | 1.2197 | 1 | 1 |
| WCM10 | 11 | 62327231 | 66461622 | −0.1364 | −1.0038 | 0.4987 | 0.4987 | 0 | 0 |
| WCM10 | 11 | 66461806 | 134605849 | 0.032 | 0.1696 | 1.1247 | 1.1247 | 1 | 1 |
| WCM10 | 12 | 148468 | 133810769 | −0.0039 | −0.0217 | 0.9851 | 0.9851 | 1 | 1 |
| WCM10 | 13 | 19041992 | 115089411 | −0.0161 | −0.0917 | 0.9384 | 0.9384 | 1 | 1 |
| WCM10 | 14 | 19110239 | 107283270 | −0.0018 | −0.0098 | 0.9932 | 0.9932 | 1 | 1 |
| WCM10 | 15 | 20083896 | 102506230 | 0.0183 | 0.0988 | 1.0709 | 1.0709 | 1 | 1 |
| WCM10 | 16 | 64432 | 28944874 | 0.008 | 0.0438 | 1.0308 | 1.0308 | 1 | 1 |
| WCM10 | 16 | 28946817 | 31276839 | −0.2292 | −2.4438 | 0.3876 | −0.02 | 0 | 0 |
| WCM10 | 16 | 31277181 | 90168646 | −0.0073 | −0.0411 | 0.9719 | 0.9719 | 1 | 1 |
| WCM10 | 17 | 131595 | 81187876 | −0.0171 | −0.0976 | 0.9346 | 0.9346 | 1 | 1 |
| WCM10 | 18 | 47738 | 78005214 | 0.0081 | 0.0445 | 1.0313 | 1.0313 | 1 | 1 |
| WCM10 | 19 | 105080 | 59093645 | −0.0447 | −0.2678 | 0.8306 | 0.8306 | 1 | 1 |
| WCM10 | 2 | 41672 | 243037103 | 0.0077 | 0.0425 | 1.0299 | 1.0299 | 1 | 1 |
| WCM10 | 20 | 68319 | 62959246 | 0.019 | 0.1028 | 1.0738 | 1.0738 | 1 | 1 |
| WCM10 | 21 | 9590331 | 48084160 | −0.0097 | −0.0549 | 0.9627 | 0.9627 | 1 | 1 |
| WCM10 | 22 | 16159031 | 51219089 | 0.0108 | 0.0589 | 1.0416 | 1.0416 | 1 | 1 |
| WCM10 | 3 | 239530 | 197896705 | 0.023 | 0.1231 | 1.0891 | 1.0891 | 1 | 1 |
| WCM10 | 4 | 53410 | 190903980 | 0.0082 | 0.0452 | 1.0319 | 1.0319 | 1 | 1 |
| WCM10 | 5 | 90714 | 180687776 | −0.002 | −0.0111 | 0.9923 | 0.9923 | 1 | 1 |
| WCM10 | 6 | 203542 | 170892848 | 0.004 | 0.022 | 1.0154 | 1.0154 | 1 | 1 |
| WCM10 | 7 | 193171 | 158937158 | 0.0161 | 0.0871 | 1.0623 | 1.0623 | 1 | 1 |
| WCM10 | 8 | 163486 | 146279416 | −0.003 | −0.0168 | 0.9884 | 0.9884 | 1 | 1 |
| WCM10 | 9 | 20703 | 141107513 | 0.0308 | 0.1632 | 1.1198 | 1.1198 | 1 | 1 |
| WCM10 | X | 2700084 | 154509348 | 0.008 | 0.0439 | NA | NA | NA | NA |
| WCM10_Z1-1 | 1 | 809001 | 55541174 | −0.0315 | −0.0718 | 0.956 | 0.9469 | 1 | 1 |
| WCM10_Z1-1 | 1 | 55542932 | 242035537 | 0.0263 | 0.0192 | 1.0197 | 1.0071 | 1 | 1 |
| WCM10_Z1-1 | 1 | 242042301 | 244527991 | −0.0089 | −0.0359 | NA | NA | NA | NA |
| WCM10_Z1-1 | 1 | 244538760 | 246498774 | −0.3468 | −0.6121 | NA | NA | NA | NA |
| WCM10_Z1-1 | 1 | 246518360 | 249212577 | 0.0482 | 0.0531 | 1.0679 | 1.0071 | 1 | 1 |
| WCM10_Z1-1 | 10 | 225988 | 135373606 | 0.0099 | −0.0065 | 1.0049 | 0.9862 | 1 | 1 |
| WCM10_Z1-1 | 11 | 193112 | 134257515 | 0.0219 | 0.0123 | 1.0164 | 1.0007 | 1 | 1 |
| WCM10_Z1-1 | 12 | 247582 | 13768577 | −0.0023 | −0.0255 | 0.9964 | 0.9686 | 1 | 1 |
| WCM10_Z1-1 | 12 | 13769433 | 25260019 | −0.4173 | −0.7461 | 1.1791 | 0.0134 | 1 | 0 |
| WCM10_Z1-1 | 12 | 25260760 | 34179803 | 0.0406 | 0.0414 | 1.045 | 1.0132 | 1 | 1 |
| WCM10_Z1-1 | 12 | 38710870 | 40499560 | −0.5148 | −0.942 | NA | NA | NA | NA |
| WCM10_Z1-1 | 12 | 40618913 | 78604198 | 0.0138 | −3.00E−04 | 0.9998 | 0.9998 | 1 | 1 |
| WCM10_Z1-1 | 12 | 79611374 | 80771821 | −0.5327 | −0.9795 | NA | NA | NA | NA |
| WCM10_Z1-1 | 12 | 81101468 | 133779274 | −0.0274 | −0.0652 | 0.9695 | 0.9421 | 1 | 1 |
| WCM10_Z1-1 | 13 | 19748104 | 47470007 | 0.037 | 0.0358 | 1.0426 | 1.0076 | 1 | 1 |
| WCM10_Z1-1 | 13 | 48523152 | 84455666 | −0.518 | −0.9486 | 1.0412 | −0.0049 | 1 | 0 |
| WCM10_Z1-1 | 13 | 86368301 | 115089411 | 0.0488 | 0.0541 | 1.0654 | 1.011 | 1 | 1 |
| WCM10_Z1-1 | 14 | 20215955 | 107283213 | 0.0053 | −0.0137 | 0.9921 | 0.989 | 1 | 1 |
| WCM10_Z1-1 | 15 | 20170134 | 102359221 | −0.1016 | −0.1849 | 0.8855 | 0.8739 | 1 | 1 |
| WCM10_Z1-1 | 16 | 97558 | 51098425 | −0.0368 | −0.0802 | 1.0063 | 0.8856 | 1 | 1 |
| WCM10_Z1-1 | 16 | 51171110 | 58768129 | −0.6048 | −1.136 | 0.976 | −0.0659 | 1 | 0 |
| WCM10_Z1-1 | 16 | 61687521 | 75281945 | −0.022 | −0.0566 | 0.9737 | 0.9493 | 1 | 1 |
| WCM10_Z1-1 | 16 | 75327888 | 75690482 | −0.556 | −1.029 | NA | NA | NA | NA |
| WCM10_Z1-1 | 16 | 76311635 | 90161798 | −0.0228 | −0.0579 | 0.9668 | 0.9546 | 1 | 1 |
| WCM10_Z1-1 | 17 | 131595 | 81052333 | −0.0414 | −0.0876 | 0.9456 | 0.9366 | 1 | 1 |
| WCM10_Z1-1 | 18 | 48177 | 28739423 | −0.0098 | −0.0373 | 0.9898 | 0.9591 | 1 | 1 |
| WCM10_Z1-1 | 18 | 28742576 | 29691838 | −0.5242 | −0.9616 | 1.0353 | −0.0084 | 1 | 0 |
| WCM10_Z1-1 | 18 | 29693820 | 78005201 | 0.0411 | 0.0421 | 1.0424 | 1.0169 | 1 | 1 |
| WCM10_Z1-1 | 19 | 281543 | 59082725 | −0.0858 | −0.1592 | 0.9305 | 0.8605 | 1 | 1 |
| WCM10_Z1-1 | 2 | 41613 | 40405612 | 0.0084 | −0.0087 | 1.0017 | 0.9862 | 1 | 1 |
| WCM10_Z1-1 | 2 | 40655772 | 48132817 | −0.576 | −1.0724 | 0.9468 | 0.0043 | 1 | 0 |
| WCM10_Z1-1 | 2 | 48573482 | 176959431 | 0.0405 | 0.0412 | 1.0354 | 1.0226 | 1 | 1 |
| WCM10_Z1-1 | 2 | 176964578 | 189876437 | 0.1554 | 0.2153 | 1.173 | 1.1489 | 1 | 1 |
| WCM10_Z1-1 | 2 | 189898749 | 242815390 | 0.0291 | 0.0235 | 1.0212 | 1.0117 | 1 | 1 |
| WCM10_Z1-1 | 20 | 68413 | 62904890 | −0.0115 | −0.04 | 0.9742 | 0.9711 | 1 | 1 |
| WCM10_Z1-1 | 21 | 10862618 | 48084207 | −0.0062 | −0.0317 | 0.9906 | 0.9659 | 1 | 1 |
| WCM10_Z1-1 | 22 | 16255872 | 51216346 | −0.0502 | −0.1016 | 0.9873 | 0.8767 | 1 | 1 |
| WCM10_Z1-1 | 3 | 361466 | 197765410 | 0.0273 | 0.0207 | 1.0192 | 1.0097 | 1 | 1 |
| WCM10_Z1-1 | 4 | 53379 | 16014921 | 0.0226 | 0.0135 | 1.0361 | 0.9827 | 1 | 1 |
| WCM10_Z1-1 | 4 | 16017861 | 21950264 | −0.4282 | −0.7672 | NA | NA | NA | NA |
| WCM10_Z1-1 | 4 | 22389379 | 115597413 | 0.049 | 0.0544 | 1.0496 | 1.0272 | 1 | 1 |
| WCM10_Z1-1 | 4 | 115750961 | 129789034 | −0.4678 | −0.8459 | 1.0565 | 0.0562 | 1 | 0 |
| WCM10_Z1-1 | 4 | 129792506 | 175899118 | 0.0824 | 0.1055 | 1.0824 | 1.0693 | 1 | 1 |
| WCM10_Z1-1 | 4 | 176556074 | 183594243 | −0.4166 | −0.7447 | NA | NA | NA | NA |
| WCM10_Z1-1 | 4 | 183600901 | 190947518 | 0.0437 | 0.0462 | 1.0517 | 1.0134 | 1 | 1 |
| WCM10_Z1-1 | 5 | 140814 | 98240677 | 0.0399 | 0.0403 | 1.0363 | 1.0203 | 1 | 1 |
| WCM10_Z1-1 | 5 | 98262023 | 102895806 | −0.3639 | −0.644 | NA | NA | NA | NA |
| WCM10_Z1-1 | 5 | 106716929 | 180687854 | 0.033 | 0.0296 | 1.0239 | 1.0176 | 1 | 1 |
| WCM10_Z1-1 | 6 | 292547 | 170892848 | 0.0481 | 0.0529 | 1.0421 | 1.0325 | 1 | 1 |
| WCM10_Z1-1 | 7 | 193657 | 6731061 | −0.0725 | −0.1376 | 0.9267 | 0.8914 | 1 | 1 |
| WCM10_Z1-1 | 7 | 6731298 | 55269511 | 0.0624 | 0.0749 | 1.0581 | 1.0484 | 1 | 1 |
| WCM10_Z1-1 | 7 | 55270250 | 76797175 | −0.0754 | −0.1423 | 0.9369 | 0.8752 | 1 | 1 |
| WCM10_Z1-1 | 7 | 76825823 | 97488205 | 0.0912 | 0.1189 | 1.1106 | 1.0612 | 1 | 1 |
| WCM10_Z1-1 | 7 | 97488569 | 97823038 | 0.0366 | 0.0351 | NA | NA | NA | NA |
| WCM10_Z1-1 | 7 | 97823249 | 102523911 | −0.0744 | −0.1407 | 1.0746 | 0.7396 | 1 | 1 |
| WCM10_Z1-1 | 7 | 102524111 | 158937416 | 0.0349 | 0.0325 | 1.0339 | 1.0117 | 1 | 1 |
| WCM10_Z1-1 | 8 | 192852 | 146279480 | 0.0286 | 0.0228 | 1.0207 | 1.0112 | 1 | 1 |
| WCM10_Z1-1 | 9 | 117642 | 2032918 | −0.078 | −0.1465 | 0.9577 | 0.8492 | 1 | 1 |
| WCM10_Z1-1 | 9 | 2039500 | 125513005 | 0.0317 | 0.0276 | 1.0209 | 1.0177 | 1 | 1 |
| WCM10_Z1-1 | 9 | 125513064 | 141016474 | −0.0338 | −0.0754 | 0.9717 | 0.9264 | 1 | 1 |
| WCM10_Z1-1 | X | 2700163 | 154509348 | 0.0164 | 0.0038 | NA | NA | NA | NA |
| WCM119 | 1 | 17465 | 119988184 | −0.1644 | −0.4482 | 0.9413 | 0.5246 | 1 | 1 |
| WCM119 | 1 | 119988585 | 152101126 | 0.1758 | 0.0826 | 1.1442 | 0.9736 | 1 | 1 |
| WCM119 | 1 | 152102620 | 153604233 | 0.505 | 0.5426 | 1.4722 | 1.441 | 1 | 1 |
| WCM119 | 1 | 153606810 | 156777073 | 0.1215 | 0.0023 | 1.0315 | 0.9717 | 1 | 1 |
| WCM119 | 1 | 156777145 | 202574734 | 0.3014 | 0.2631 | 1.2 | 1.2 | 1 | 1 |
| WCM119 | 1 | 202696570 | 249214028 | 0.2205 | 0.1475 | 1.1077 | 1.1077 | 1 | 1 |
| WCM119 | 10 | 226107 | 3827144 | −0.366 | −0.8031 | 0.6285 | 0.5177 | 1 | 1 |
| WCM119 | 10 | 4702299 | 44865739 | 0.1071 | −0.0193 | 0.9867 | 0.9867 | 1 | 1 |
| WCM119 | 10 | 44866751 | 46217168 | −0.4747 | −1.0121 | NA | NA | NA | NA |
| WCM119 | 10 | 46217755 | 60129301 | 0.2121 | 0.1354 | 1.1016 | 1.0952 | 1 | 1 |
| WCM119 | 10 | 60130322 | 63816876 | −0.4221 | −0.9092 | NA | NA | NA | NA |
| WCM119 | 10 | 63817096 | 69905295 | 0.05 | −0.1058 | NA | NA | NA | NA |
| WCM119 | 10 | 69908083 | 71851670 | −0.4235 | −0.9119 | 1.066 | −0.003 | 1 | 0 |
| WCM119 | 10 | 71851726 | 89312053 | 0.14 | 0.0297 | 1.0208 | 1.0208 | 1 | 1 |
| WCM119 | 10 | 89419781 | 90122432 | −1.515 | NA | NA | NA | NA | NA |
| WCM119 | 10 | 90332708 | 135477913 | 0.0928 | −0.0408 | 0.9721 | 0.9721 | 1 | 1 |
| WCM119 | 11 | 193096 | 62066474 | 0.1506 | 0.0454 | 1.032 | 1.032 | 1 | 1 |
| WCM119 | 11 | 62105609 | 76163008 | −0.0245 | −0.2213 | 0.8578 | 0.8578 | 1 | 1 |
| WCM119 | 11 | 76164325 | 118771385 | 0.144 | 0.0357 | 1.025 | 1.025 | 1 | 1 |
| WCM119 | 11 | 118771708 | 122955468 | −0.14 | −0.4076 | 0.8249 | 0.6828 | 1 | 1 |
| WCM119 | 11 | 122968481 | 134605959 | 0.162 | 0.0622 | 1.044 | 1.044 | 1 | 1 |
| WCM119 | 12 | 148480 | 3407389 | 0.0951 | −0.0373 | 0.9745 | 0.9745 | 1 | 1 |
| WCM119 | 12 | 3409239 | 16757863 | −0.4211 | −0.9073 | 1.046 | 0.0203 | 1 | 0 |
| WCM119 | 12 | 18234097 | 111472030 | 0.1138 | −0.0092 | 0.9936 | 0.9936 | 1 | 1 |
| WCM119 | 12 | 111511888 | 133810883 | −0.5158 | −1.0952 | 0.9666 | −0.0305 | 1 | 0 |
| WCM119 | 13 | 19041979 | 115089411 | −0.1596 | −0.4402 | 0.9104 | 0.5636 | 1 | 1 |
| WCM119 | 14 | 19110188 | 68228217 | −0.191 | −0.493 | 0.903 | 0.518 | 1 | 1 |
| WCM119 | 14 | 68228977 | 70243006 | −0.4891 | −1.041 | 0.9586 | 0.0134 | 1 | 0 |
| WCM119 | 14 | 70245147 | 107283088 | −0.1706 | −0.4585 | 0.9254 | 0.53 | 1 | 1 |
| WCM119 | 15 | 20083896 | 64747160 | 0.308 | 0.2723 | 1.3489 | 1.0666 | 1 | 1 |
| WCM119 | 15 | 64791668 | 80263484 | −0.5076 | −1.0784 | 0.9315 | 0.0156 | 1 | 0 |
| WCM119 | 15 | 80412697 | 102514186 | 0.0704 | −0.0746 | 0.9496 | 0.9496 | 1 | 1 |
| WCM119 | 16 | 64429 | 34277581 | 0.1031 | −0.0254 | 0.9826 | 0.9826 | 1 | 1 |
| WCM119 | 16 | 34277902 | 90260461 | −0.5102 | −1.0839 | 0.9443 | −7.00E−04 | 1 | 0 |
| WCM119 | 17 | 131595 | 4585911 | 0.0534 | −0.1006 | 0.9326 | 0.9326 | 1 | 1 |
| WCM119 | 17 | 4586161 | 16537207 | −0.5093 | −1.082 | 0.9307 | 0.014 | 1 | 0 |
| WCM119 | 17 | 16537987 | 31618703 | 0.0372 | −0.1253 | 0.9168 | 0.9168 | 1 | 1 |
| WCM119 | 17 | 32201294 | 36248549 | −0.4898 | −1.0424 | 0.9618 | 0.0092 | 1 | 0 |
| WCM119 | 17 | 36339813 | 39341158 | −0.1969 | −0.5029 | 0.8267 | 0.5846 | 1 | 1 |
| WCM119 | 17 | 39341473 | 43186394 | −0.4547 | −0.9726 | 0.8633 | 0.1558 | 1 | 0 |
| WCM119 | 17 | 43189953 | 63133674 | −0.1774 | −0.4701 | 0.932 | 0.5118 | 1 | 1 |
| WCM119 | 17 | 63149650 | 81187935 | 0.0787 | −0.062 | 1.422 | 0.4938 | 1 | 0 |
| WCM119 | 18 | 47632 | 61602369 | 0.0886 | −0.0471 | 0.9679 | 0.9679 | 1 | 1 |
| WCM119 | 18 | 61620715 | 78005197 | −0.5129 | −1.0894 | 0.906 | 0.034 | 1 | 0 |
| WCM119 | 19 | 104963 | 4095331 | −0.1703 | −0.4581 | 0.8317 | 0.6242 | 1 | 1 |
| WCM119 | 19 | 4097321 | 4685797 | 0.3494 | 0.3302 | 1.3429 | 1.1714 | 1 | 1 |
| WCM119 | 19 | 4689534 | 10394391 | −0.0912 | −0.3277 | 0.9425 | 0.6511 | 1 | 1 |
| WCM119 | 19 | 10394764 | 13319842 | 0.0597 | −0.0909 | 0.9733 | 0.9046 | 1 | 1 |
| WCM119 | 19 | 13321480 | 23317042 | 0.2858 | 0.2409 | 1.1968 | 1.1667 | 1 | 1 |
| WCM119 | 19 | 23318731 | 58920263 | 0.085 | −0.0525 | 0.9643 | 0.9643 | 1 | 1 |
| WCM119 | 19 | 58921455 | 59093696 | −0.1978 | −0.5045 | NA | NA | NA | NA |
| WCM119 | 2 | 41595 | 8952612 | −0.1616 | −0.4436 | 0.7881 | 0.6825 | 1 | 1 |
| WCM119 | 2 | 8953489 | 10583643 | −0.557 | −1.1812 | 0.9264 | −0.0444 | 1 | 0 |
| WCM119 | 2 | 10583865 | 20518340 | −0.1223 | −0.3784 | 0.9459 | 0.5926 | 1 | 1 |
| WCM119 | 2 | 20525948 | 20649195 | −1.3933 | −5.0442 | NA | NA | NA | NA |
| WCM119 | 2 | 20699667 | 25945260 | −0.1476 | −0.4202 | 0.9866 | 0.508 | 1 | 1 |
| WCM119 | 2 | 25964713 | 26750645 | −0.4629 | −0.9888 | 1.0211 | −0.0132 | 1 | 0 |
| WCM119 | 2 | 26760649 | 216002860 | −0.1507 | −0.4254 | 0.9562 | 0.5331 | 1 | 1 |
| WCM119 | 2 | 216177303 | 218620823 | −0.9022 | −2.0448 | 0.5047 | −0.02 | 1 | 0 |
| WCM119 | 2 | 218667474 | 243052762 | −0.1472 | −0.4195 | 0.8953 | 0.6001 | 1 | 1 |
| WCM119 | 20 | 68406 | 348183 | −0.3474 | −0.7687 | NA | NA | NA | NA |
| WCM119 | 20 | 348244 | 34220224 | −0.1449 | −0.4158 | 0.9645 | 0.5347 | 1 | 1 |
| WCM119 | 20 | 34220601 | 47558574 | −0.4609 | −0.9847 | 1.0312 | −0.0205 | 1 | 0 |
| WCM119 | 20 | 47567811 | 62934623 | 0.3016 | 0.2633 | 1.2205 | 1.1799 | 1 | 1 |
| WCM119 | 21 | 9590369 | 34717604 | −0.1604 | −0.4414 | 0.9207 | 0.5521 | 1 | 1 |
| WCM119 | 21 | 34721524 | 39823096 | −0.312 | −0.7043 | 0.6868 | 0.5407 | 1 | 1 |
| WCM119 | 21 | 39870310 | 41300922 | −0.9834 | −2.3067 | 0.4286 | −0.0243 | 0 | 0 |
| WCM119 | 21 | 41301084 | 42870017 | −0.4936 | −1.0502 | 1.0028 | −0.037 | 1 | 0 |
| WCM119 | 21 | 43133055 | 48084177 | 0.0839 | −0.0542 | 0.9631 | 0.9631 | 1 | 1 |
| WCM119 | 22 | 16159043 | 51219129 | 0.0578 | −0.0939 | 0.937 | 0.937 | 1 | 1 |
| WCM119 | 3 | 239531 | 1427204 | 0.2511 | 0.1916 | NA | NA | NA | NA |
| WCM119 | 3 | 1427450 | 11059596 | 0.0215 | −0.1496 | 0.9182 | 0.8848 | 1 | 1 |
| WCM119 | 3 | 11059732 | 12653407 | −0.4855 | −1.0338 | 0.9848 | −0.0079 | 1 | 0 |
| WCM119 | 3 | 12660127 | 154898748 | 0.3294 | 0.3023 | 1.2331 | 1.2331 | 1 | 1 |
| WCM119 | 3 | 154983068 | 179132727 | 0.4988 | 0.5343 | 1.4482 | 1.4482 | 1 | 1 |
| WCM119 | 3 | 179134323 | 197896656 | 0.3176 | 0.2858 | 1.2345 | 1.2037 | 1 | 1 |
| WCM119 | 4 | 53375 | 190904455 | −0.1776 | −0.4703 | 0.9161 | 0.5275 | 1 | 1 |
| WCM119 | 5 | 90537 | 9066764 | 0.0846 | −0.0531 | 0.9638 | 0.9638 | 1 | 1 |
| WCM119 | 5 | 9108424 | 70800475 | −0.1567 | −0.4354 | 0.8541 | 0.6249 | 1 | 1 |
| WCM119 | 5 | 70805422 | 180687828 | 0.0774 | −0.064 | 0.9566 | 0.9566 | 1 | 1 |
| WCM119 | 6 | 203475 | 67859495 | 0.0762 | −0.0659 | 0.9554 | 0.9554 | 1 | 1 |
| WCM119 | 6 | 69345838 | 170892697 | −0.1449 | −0.4157 | 0.936 | 0.5633 | 1 | 1 |
| WCM119 | 7 | 193205 | 158937018 | 0.1294 | 0.0141 | 1.0098 | 1.0098 | 1 | 1 |
| WCM119 | 8 | 163438 | 18391899 | −0.2683 | −0.6263 | 0.6604 | 0.6353 | 1 | 1 |
| WCM119 | 8 | 18392379 | 41836324 | −0.6711 | −1.4344 | 0.7193 | 0.0207 | 1 | 0 |
| WCM119 | 8 | 41838344 | 146279399 | 0.0494 | −0.1067 | 0.9287 | 0.9287 | 1 | 1 |
| WCM119 | 9 | 14878 | 97177479 | 0.0899 | −0.0451 | 0.9692 | 0.9692 | 1 | 1 |
| WCM119 | 9 | 97191424 | 99521494 | −0.4851 | −1.0329 | 0.9509 | 0.0266 | 1 | 0 |
| WCM119 | 9 | 99521523 | 141107525 | 0.0883 | −0.0476 | 0.9676 | 0.9676 | 1 | 1 |
| WCM119 | X | 2700202 | 9000969 | −0.3646 | −0.8004 | NA | NA | NA | NA |
| WCM119 | X | 9622286 | 12712534 | 0.4274 | 0.4377 | NA | NA | NA | NA |
| WCM119 | X | 12720157 | 13774915 | −0.4728 | −1.0085 | NA | NA | NA | NA |
| WCM119 | X | 13775867 | 20283620 | 0.5766 | 0.6375 | NA | NA | NA | NA |
| WCM119 | X | 20590519 | 26236216 | 1.1069 | 1.3019 | NA | NA | NA | NA |
| WCM119 | X | 27479248 | 43603422 | −0.4495 | −0.9624 | NA | NA | NA | NA |
| WCM119 | X | 43603715 | 57937046 | 1.0293 | 1.2083 | NA | NA | NA | NA |
| WCM119 | X | 61998801 | 69176915 | 2.1395 | 2.4748 | NA | NA | NA | NA |
| WCM119 | X | 69178468 | 73069575 | 0.802 | 0.9276 | NA | NA | NA | NA |
| WCM119 | X | 73070031 | 75004943 | 0.4132 | 0.4183 | NA | NA | NA | NA |
| WCM119 | X | 75393441 | 101821867 | −0.4297 | −0.9239 | NA | NA | NA | NA |
| WCM119 | X | 101822823 | 102613323 | 1.134 | 1.3344 | NA | NA | NA | NA |
| WCM119 | X | 102632519 | 119761006 | 0.5011 | 0.5372 | NA | NA | NA | NA |
| WCM119 | X | 120008890 | 132162272 | −0.5008 | −1.0647 | NA | NA | NA | NA |
| WCM119 | X | 132351105 | 134126297 | 0.5541 | 0.6079 | NA | NA | NA | NA |
| WCM119 | X | 134155965 | 135496525 | −0.4315 | −0.9273 | NA | NA | NA | NA |
| WCM119 | X | 135498568 | 137714138 | 0.5572 | 0.6119 | NA | NA | NA | NA |
| WCM119 | X | 137714483 | 154493609 | −0.4249 | −0.9145 | NA | NA | NA | NA |
| WCM119_Z6_1 | 1 | 69594 | 120050152 | −0.216 | 0.6223 | 2.0706 | 1.008 | 2 | 1 |
| WCM119_Z6_1 | 1 | 120056648 | 249212429 | 0.3725 | 1.3395 | 3.0454 | 2.0157 | 3 | 2 |
| WCM119_Z6_1 | 10 | 225960 | 468916 | −0.7159 | −0.0544 | NA | NA | NA | NA |
| WCM119_Z6_1 | 10 | 486884 | 44345143 | 0.1454 | 1.0701 | 2.1273 | 2.0717 | 2 | 2 |
| WCM119_Z6_1 | 10 | 44866808 | 47769286 | −0.4853 | 0.2681 | NA | NA | NA | NA |
| WCM119_Z6_1 | 10 | 48370587 | 60272976 | 0.1294 | 1.0508 | 2.1016 | 2.0416 | 2 | 2 |
| WCM119_Z6_1 | 10 | 60380631 | 63450323 | −0.6475 | 0.0434 | NA | NA | NA | NA |
| WCM119_Z6_1 | 10 | 63519858 | 69905277 | 0.1094 | 1.0266 | NA | NA | NA | NA |
| WCM119_Z6_1 | 10 | 69908187 | 71718455 | −0.557 | 0.17 | 2.2141 | 0.036 | 2 | 0 |
| WCM119_Z6_1 | 10 | 71835546 | 89481518 | 0.1524 | 1.0785 | 2.1449 | 2.0789 | 2 | 2 |
| WCM119_Z6_1 | 10 | 89487142 | 90356651 | −1.7977 | −2.1273 | NA | NA | NA | NA |
| WCM119_Z6_1 | 10 | 90362375 | 135440086 | 0.1455 | 1.0702 | 2.1225 | 2.077 | 2 | 2 |
| WCM119_Z6_1 | 11 | 193112 | 71293558 | 0.101 | 1.0164 | 2.045 | 2.0009 | 2 | 2 |
| WCM119_Z6_1 | 11 | 71293677 | 75148030 | −0.2198 | 0.6174 | 2.0385 | 1.0297 | 2 | 1 |
| WCM119_Z6_1 | 11 | 75150397 | 118851234 | 0.0994 | 1.0145 | 2.057 | 1.9835 | 2 | 2 |
| WCM119_Z6_1 | 11 | 118867695 | 122165645 | −0.2548 | 0.5725 | 1.8435 | 1.1308 | 2 | 1 |
| WCM119_Z6_1 | 11 | 122526799 | 134856523 | 0.1099 | 1.0272 | 2.0947 | 1.9813 | 2 | 2 |
| WCM119_Z6_1 | 12 | 148675 | 3409304 | 0.2077 | 1.1448 | NA | NA | NA | NA |
| WCM119_Z6_1 | 12 | 3490591 | 17142459 | −0.6653 | 0.0182 | 2.1009 | −0.0755 | 2 | 0 |
| WCM119_Z6_1 | 12 | 17826233 | 41946470 | 0.0821 | 0.9934 | 2.0296 | 1.9522 | 2 | 2 |
| WCM119_Z6_1 | 12 | 41949551 | 68549144 | 0.0162 | 0.913 | 1.9177 | 1.8483 | 2 | 2 |
| WCM119_Z6_1 | 12 | 68551734 | 111375170 | 0.1137 | 1.0318 | 2.0742 | 2.0149 | 2 | 2 |
| WCM119_Z6_1 | 12 | 111471998 | 133810703 | −0.6621 | 0.0228 | 2.0285 | 0.0033 | 2 | 0 |
| WCM119_Z6_1 | 13 | 19600475 | 115089411 | −0.2254 | 0.6102 | 2.0759 | 0.9771 | 2 | 1 |
| WCM119_Z6_1 | 14 | 19377697 | 68222802 | −0.2316 | 0.6023 | 2.0301 | 1.0061 | 2 | 1 |
| WCM119_Z6_1 | 14 | 68228028 | 70175757 | −0.6466 | 0.0447 | NA | NA | NA | NA |
| WCM119_Z6_1 | 14 | 70177691 | 107283213 | −0.2195 | 0.6178 | 2.0862 | 0.9829 | 2 | 1 |
| WCM119_Z6_1 | 15 | 20170018 | 45962168 | 0.5568 | 1.5527 | 3.011 | 2.8563 | 3 | 3 |
| WCM119_Z6_1 | 15 | 45965880 | 64747240 | 0.1306 | 1.0522 | 3.0721 | 1.0753 | 3 | 1 |
| WCM119_Z6_1 | 15 | 64791829 | 80260029 | −1.2954 | −0.9923 | 1.0707 | −0.0654 | 1 | 0 |
| WCM119_Z6_1 | 15 | 80263171 | 102517639 | −0.2203 | 0.6168 | 2.077 | 0.99 | 2 | 1 |
| WCM119_Z6_1 | 16 | 66966 | 33965560 | 0.116 | 1.0346 | 2.0931 | 2.004 | 2 | 2 |
| WCM119_Z6_1 | 16 | 34257013 | 90233596 | −0.6592 | 0.0268 | 2.0666 | −0.0291 | 2 | 0 |
| WCM119_Z6_1 | 17 | 131595 | 4586185 | 0.0892 | 1.0021 | 2.0539 | 1.9518 | 2 | 2 |
| WCM119_Z6_1 | 17 | 4594269 | 16538031 | −0.6547 | 0.0333 | 2.0745 | −0.0278 | 2 | 0 |
| WCM119_Z6_1 | 17 | 16550679 | 31618443 | 0.0953 | 1.0095 | 2.0548 | 1.9717 | 2 | 2 |
| WCM119_Z6_1 | 17 | 31618866 | 36353758 | −0.6864 | −0.0119 | 1.9889 | −0.0053 | 2 | 0 |
| WCM119_Z6_1 | 17 | 36357230 | 39086290 | −0.2534 | 0.5743 | 1.9971 | 0.9808 | 2 | 1 |
| WCM119_Z6_1 | 17 | 39114868 | 41020861 | −0.6876 | −0.0136 | 2.0181 | −0.0369 | 2 | 0 |
| WCM119_Z6_1 | 17 | 41031597 | 42287770 | −0.3609 | 0.4343 | 1.8531 | 0.8493 | 2 | 1 |
| WCM119_Z6_1 | 17 | 42288288 | 43182993 | −0.6686 | 0.0136 | 2.0434 | −0.0245 | 2 | 0 |
| WCM119_Z6_1 | 17 | 43190066 | 63746821 | −0.2422 | 0.5887 | 2.0276 | 0.9802 | 2 | 1 |
| WCM119_Z6_1 | 17 | 63822329 | 81083567 | 0.1003 | 1.0156 | 3.0533 | 0.9902 | 3 | 1 |
| WCM119_Z6_1 | 18 | 48202 | 61602462 | 0.1225 | 1.0424 | 2.092 | 2.0273 | 2 | 2 |
| WCM119_Z6_1 | 18 | 61620752 | 78005214 | −0.6955 | −0.025 | 2.0625 | −0.0969 | 2 | 0 |
| WCM119_Z6_1 | 19 | 281468 | 3478866 | −0.2648 | 0.5596 | 2.0104 | 0.9374 | 2 | 1 |
| WCM119_Z6_1 | 19 | 3480448 | 3700307 | 0.3668 | 1.3328 | NA | NA | NA | NA |
| WCM119_Z6_1 | 19 | 3728440 | 4090617 | −0.3013 | 0.5122 | 1.9048 | 0.9477 | 2 | 1 |
| WCM119_Z6_1 | 19 | 4094487 | 4685563 | 0.3231 | 1.2815 | 2.5539 | 2.308 | 3 | 2 |
| WCM119_Z6_1 | 19 | 4688845 | 10341096 | −0.2708 | 0.5519 | 2.0021 | 0.9298 | 2 | 1 |
| WCM119_Z6_1 | 19 | 10362984 | 11687196 | 0.3768 | 1.3444 | 3.0081 | 2.0703 | 3 | 2 |
| WCM119_Z6_1 | 19 | 11687534 | 12719976 | −0.3225 | 0.4847 | 1.9751 | 0.8235 | 2 | 1 |
| WCM119_Z6_1 | 19 | 12720757 | 20308715 | 0.3368 | 1.2975 | 2.8189 | 2.0973 | 3 | 2 |
| WCM119_Z6_1 | 19 | 20308825 | 59083988 | 0.0557 | 0.9613 | 1.9756 | 1.9186 | 2 | 2 |
| WCM119_Z6_1 | 2 | 41620 | 8926125 | −0.2608 | 0.5648 | 1.855 | 1.1033 | 2 | 1 |
| WCM119_Z6_1 | 2 | 8926425 | 10564692 | −0.6761 | 0.0028 | NA | NA | NA | NA |
| WCM119_Z6_1 | 2 | 10566887 | 25803648 | −0.2543 | 0.5731 | 2.0358 | 0.9396 | 2 | 1 |
| WCM119_Z6_1 | 2 | 25819081 | 26804247 | −0.6788 | −0.001 | 2.043 | −0.0445 | 2 | 0 |
| WCM119_Z6_1 | 2 | 26805757 | 216002898 | −0.2275 | 0.6076 | 2.0372 | 1.0103 | 2 | 1 |
| WCM119_Z6_1 | 2 | 216176870 | 218620794 | −1.2937 | −0.9891 | NA | NA | NA | NA |
| WCM119_Z6_1 | 2 | 218669179 | 242946607 | −0.2221 | 0.6144 | 2.0365 | 1.0254 | 2 | 1 |
| WCM119_Z6_1 | 20 | 68363 | 34858532 | −0.2165 | 0.6217 | 2.0561 | 1.0213 | 2 | 1 |
| WCM119_Z6_1 | 20 | 35060162 | 47557736 | −0.6738 | 0.0061 | 2.023 | −0.0145 | 2 | 0 |
| WCM119_Z6_1 | 20 | 47558464 | 62904866 | 0.4289 | 1.4051 | 3.2022 | 2.0945 | 3 | 2 |
| WCM119_Z6_1 | 21 | 9826205 | 39822803 | −0.2317 | 0.6022 | 2.0339 | 1.0022 | 2 | 1 |
| WCM119_Z6_1 | 21 | 39947620 | 43133720 | −1.2677 | −0.9415 | 0.9621 | 0.0793 | 1 | 0 |
| WCM119_Z6_1 | 21 | 43134230 | 48084204 | −0.2078 | 0.6328 | 1.9879 | 1.1131 | 2 | 1 |
| WCM119_Z6_1 | 22 | 16287478 | 51215134 | 0.1015 | 1.017 | 2.0385 | 2.0091 | 2 | 2 |
| WCM119_Z6_1 | 3 | 239436 | 10861246 | 0.0997 | 1.0149 | 2.1187 | 1.9228 | 2 | 2 |
| WCM119_Z6_1 | 3 | 10861450 | 12653531 | −0.7027 | −0.0354 | NA | NA | NA | NA |
| WCM119_Z6_1 | 3 | 12660201 | 197894720 | 0.3793 | 1.3473 | 3.0112 | 2.0776 | 3 | 2 |
| WCM119_Z6_1 | 4 | 59397 | 1087555 | −0.4523 | 0.3126 | 1.3104 | 1.1735 | 1 | 1 |
| WCM119_Z6_1 | 4 | 1090625 | 87622684 | −0.3314 | 0.4731 | 1.7678 | 1.0084 | 2 | 1 |
| WCM119_Z6_1 | 4 | 87622942 | 190426502 | −0.4853 | 0.2681 | 1.4361 | 0.9724 | 1 | 1 |
| WCM119_Z6_1 | 5 | 140657 | 180687804 | −0.2246 | 0.6112 | 2.042 | 1.0131 | 2 | 1 |
| WCM119_Z6_1 | 6 | 203515 | 65612302 | 0.3721 | 1.3389 | 3.0237 | 2.0356 | 3 | 2 |
| WCM119_Z6_1 | 6 | 65622463 | 170893411 | −0.2291 | 0.6055 | 2.0207 | 1.0223 | 2 | 1 |
| WCM119_Z6_1 | 7 | 193205 | 43436424 | 0.0238 | 0.9224 | 1.9324 | 1.858 | 2 | 2 |
| WCM119_Z6_1 | 7 | 43447302 | 128506750 | 0.1906 | 1.1244 | 2.3027 | 2.0575 | 2 | 2 |
| WCM119_Z6_1 | 7 | 128508812 | 158937400 | 0.0225 | 0.9207 | 2.1252 | 1.6609 | 2 | 2 |
| WCM119_Z6_1 | 8 | 190908 | 19297347 | 0.0976 | 1.0123 | 2.0954 | 1.9389 | 2 | 2 |
| WCM119_Z6_1 | 8 | 19315321 | 41906095 | −0.6871 | −0.0129 | 2.0416 | −0.0595 | 2 | 0 |
| WCM119_Z6_1 | 8 | 41906261 | 146279514 | 0.1661 | 1.095 | 2.1848 | 2.0874 | 2 | 2 |
| WCM119_Z6_1 | 9 | 17055 | 97177479 | 0.1027 | 1.0185 | 2.0528 | 1.9988 | 2 | 2 |
| WCM119_Z6_1 | 9 | 97203361 | 99417036 | −0.6616 | 0.0235 | NA | NA | NA | NA |
| WCM119_Z6_1 | 9 | 99521302 | 141111414 | 0.0999 | 1.0151 | 2.0553 | 1.9867 | 2 | 2 |
| WCM119_Z6_1 | X | 2707700 | 9001007 | −0.582 | 0.1354 | NA | NA | NA | NA |
| WCM119_Z6_1 | X | 9621715 | 11793148 | 0.591 | 1.5918 | NA | NA | NA | NA |
| WCM119_Z6_1 | X | 12632953 | 13769388 | −0.5947 | 0.1177 | NA | NA | NA | NA |
| WCM119_Z6_1 | X | 13771497 | 20194447 | 0.5616 | 1.5582 | NA | NA | NA | NA |
| WCM119_Z6_1 | X | 20206041 | 25031901 | 1.2151 | 2.2862 | NA | NA | NA | NA |
| WCM119_Z6_1 | X | 25033689 | 26236115 | 0.6384 | 1.6459 | NA | NA | NA | NA |
| WCM119_Z6_1 | X | 27478926 | 28807508 | −0.6034 | 0.1055 | NA | NA | NA | NA |
| WCM119_Z6_1 | X | 29301057 | 30577652 | 0.4976 | 1.4847 | NA | NA | NA | NA |
| WCM119_Z6_1 | X | 30578153 | 42637311 | −0.7136 | −0.051 | NA | NA | NA | NA |
| WCM119_Z6_1 | X | 43542795 | 44037679 | 0.4331 | 1.41 | NA | NA | NA | NA |
| WCM119_Z6_1 | X | 44088925 | 57936447 | 1.2033 | 2.2734 | NA | NA | NA | NA |
| WCM119_Z6_1 | X | 62898360 | 69176971 | 2.2368 | 3.3709 | NA | NA | NA | NA |
| WCM119_Z6_1 | X | 69242767 | 73094914 | 1.0379 | 2.0924 | NA | NA | NA | NA |
| WCM119_Z6_1 | X | 73095472 | 75396725 | 0.4623 | 1.4439 | NA | NA | NA | NA |
| WCM119_Z6_1 | X | 75397840 | 101581385 | −0.6649 | 0.0187 | NA | NA | NA | NA |
| WCM119_Z6_1 | X | 101857242 | 102632712 | 1.2928 | 2.3705 | NA | NA | NA | NA |
| WCM119_Z6_1 | X | 102831404 | 107627026 | 0.6078 | 1.611 | NA | NA | NA | NA |
| WCM119_Z6_1 | X | 107683386 | 107979388 | −0.7419 | −0.0921 | NA | NA | NA | NA |
| WCM119_Z6_1 | X | 108619152 | 111698569 | 0.5926 | 1.5937 | NA | NA | NA | NA |
| WCM119_Z6_1 | X | 111698889 | 117480524 | −0.6928 | −0.0211 | NA | NA | NA | NA |
| WCM119_Z6_1 | X | 117526683 | 119760973 | 0.5799 | 1.5791 | NA | NA | NA | NA |
| WCM119_Z6_1 | X | 120008749 | 132162173 | −0.6683 | 0.0139 | NA | NA | NA | NA |
| WCM119_Z6_1 | X | 132351220 | 134186126 | 0.5358 | 1.5286 | NA | NA | NA | NA |
| WCM119_Z6_1 | X | 134303518 | 135496346 | −0.7627 | −0.1224 | NA | NA | NA | NA |
| WCM119_Z6_1 | X | 135519177 | 136659281 | 0.5855 | 1.5856 | NA | NA | NA | NA |
| WCM119_Z6_1 | X | 137715128 | 154509348 | −0.674 | 0.0058 | NA | NA | NA | NA |
| WCM12 | 1 | 14930 | 249213967 | 0.0169 | 0.0963 | 1.0691 | 1.0691 | 1 | 1 |
| WCM12 | 10 | 225936 | 135439008 | 0.0148 | 0.0765 | 1.0544 | 1.0544 | 1 | 1 |
| WCM12 | 11 | 193096 | 62327920 | 0.0362 | 0.2682 | 1.2655 | 1.1431 | 1 | 1 |
| WCM12 | 11 | 62334460 | 65827316 | −0.1283 | −3.254 | 0.2232 | −0.0135 | 0 | 0 |
| WCM12 | 11 | 65828095 | 134605906 | 0.0236 | 0.1578 | 1.1156 | 1.1156 | 1 | 1 |
| WCM12 | 12 | 148560 | 133810703 | −0.0025 | −0.098 | 0.9343 | 0.9343 | 1 | 1 |
| WCM12 | 13 | 19041937 | 115089411 | −0.0097 | −0.1763 | 0.885 | 0.885 | 1 | 1 |
| WCM12 | 14 | 19110387 | 107283270 | −2.00E−04 | −0.0732 | 0.9505 | 0.9505 | 1 | 1 |
| WCM12 | 15 | 20083896 | 102516308 | 0.0266 | 0.1847 | 1.1771 | 1.096 | 1 | 1 |
| WCM12 | 16 | 66882 | 90260461 | −0.0016 | −0.0885 | 0.9405 | 0.9405 | 1 | 1 |
| WCM12 | 17 | 131595 | 81187813 | −0.0095 | −0.1737 | 0.8866 | 0.8866 | 1 | 1 |
| WCM12 | 18 | 47824 | 78005110 | 0.0052 | −0.0178 | 0.9877 | 0.9877 | 1 | 1 |
| WCM12 | 19 | 104963 | 59093696 | −0.0332 | −0.4638 | 0.7251 | 0.7251 | 1 | 1 |
| WCM12 | 2 | 41602 | 243052710 | −0.004 | −0.1136 | 0.9243 | 0.9243 | 1 | 1 |
| WCM12 | 20 | 68319 | 62934618 | 0.0208 | 0.132 | 1.0958 | 1.0958 | 1 | 1 |
| WCM12 | 21 | 9590271 | 48084253 | −0.0113 | −0.194 | 0.8742 | 0.8742 | 1 | 1 |
| WCM12 | 22 | 16159046 | 51223730 | 0.0405 | 0.3042 | 1.2757 | 1.1938 | 1 | 1 |
| WCM12 | 3 | 239390 | 197896801 | 0.0094 | 0.0237 | 1.0166 | 1.0166 | 1 | 1 |
| WCM12 | 4 | 53446 | 190874234 | −0.0037 | −0.1112 | 0.9258 | 0.9258 | 1 | 1 |
| WCM12 | 5 | 90684 | 180687812 | −0.0115 | −0.1966 | 0.8726 | 0.8726 | 1 | 1 |
| WCM12 | 6 | 203369 | 170892877 | −0.006 | −0.136 | 0.91 | 0.91 | 1 | 1 |
| WCM12 | 7 | 195702 | 158937158 | 0.0022 | −0.0489 | 1.0065 | 0.9268 | 1 | 1 |
| WCM12 | 8 | 163432 | 146279453 | −0.0037 | −0.1108 | 0.9261 | 0.9261 | 1 | 1 |
| WCM12 | 9 | 116800 | 141107539 | 0.0204 | 0.129 | 1.0935 | 1.0935 | 1 | 1 |
| WCM12 | X | 2700141 | 154507335 | −0.0248 | −0.3548 | NA | NA | NA | NA |
| WCM121 | 1 | 14907 | 249213964 | 0.0191 | NA | NA | NA | NA | NA |
| WCM121 | 10 | 226116 | 135438973 | 0.0155 | NA | NA | NA | NA | NA |
| WCM121 | 11 | 193096 | 134605916 | 0.0189 | NA | NA | NA | NA | NA |
| WCM121 | 12 | 148580 | 133810905 | 0.0052 | NA | NA | NA | NA | NA |
| WCM121 | 13 | 19041979 | 115089411 | −0.0043 | NA | NA | NA | NA | NA |
| WCM121 | 14 | 19110178 | 107283266 | 0.0068 | NA | NA | NA | NA | NA |
| WCM121 | 15 | 20083896 | 102514188 | 0.0233 | NA | NA | NA | NA | NA |
| WCM121 | 16 | 66921 | 90260461 | 0.015 | NA | NA | NA | NA | NA |
| WCM121 | 17 | 131595 | 81187934 | 0.0029 | NA | NA | NA | NA | NA |
| WCM121 | 18 | 47654 | 78005203 | 0.0103 | NA | NA | NA | NA | NA |
| WCM121 | 19 | 105021 | 59093645 | 0.0047 | NA | NA | NA | NA | NA |
| WCM121 | 2 | 41614 | 243037022 | 0.0044 | NA | NA | NA | NA | NA |
| WCM121 | 20 | 68311 | 62934618 | 0.0353 | NA | NA | NA | NA | NA |
| WCM121 | 21 | 9590271 | 48084114 | 0.0055 | NA | NA | NA | NA | NA |
| WCM121 | 22 | 16159043 | 51219089 | 0.0517 | NA | NA | NA | NA | NA |
| WCM121 | 3 | 239495 | 197896720 | 0.016 | NA | NA | NA | NA | NA |
| WCM121 | 4 | 53397 | 190904468 | 0.0033 | NA | NA | NA | NA | NA |
| WCM121 | 5 | 90696 | 180687861 | 0.0046 | NA | NA | NA | NA | NA |
| WCM121 | 6 | 203510 | 170892854 | 1.00E−04 | NA | NA | NA | NA | NA |
| WCM121 | 7 | 193253 | 158937142 | 0.0191 | NA | NA | NA | NA | NA |
| WCM121 | 8 | 163491 | 146279507 | 0.0164 | NA | NA | NA | NA | NA |
| WCM121 | 9 | 14893 | 141107468 | 0.0216 | NA | NA | NA | NA | NA |
| WCM121 | X | 2700122 | 154493585 | −0.0838 | NA | NA | NA | NA | NA |
| WCM121_Z1_1 | 1 | 721439 | 23696165 | −0.4129 | 0.0473 | 1.2458 | 0.8209 | 1 | 1 |
| WCM121_Z1_1 | 1 | 23697698 | 28835161 | −0.1586 | 0.5836 | 1.9881 | 1.0091 | 2 | 1 |
| WCM121_Z1_1 | 1 | 28835457 | 75184893 | 0.0727 | 0.9995 | 2.0365 | 1.9622 | 2 | 2 |
| WCM121_Z1_1 | 1 | 75188910 | 120458366 | −0.1291 | 0.6399 | 2.0667 | 1.0497 | 2 | 1 |
| WCM121_Z1_1 | 1 | 120458537 | 151735425 | 0.0966 | 1.0397 | 2.9941 | 1.1176 | 3 | 1 |
| WCM121_Z1_1 | 1 | 151735817 | 179562992 | −0.142 | 0.6155 | 2.0273 | 1.0369 | 2 | 1 |
| WCM121_Z1_1 | 1 | 179587734 | 205686457 | 0.0899 | 1.0285 | 2.1015 | 1.9784 | 2 | 2 |
| WCM121_Z1_1 | 1 | 205687444 | 249150528 | −0.1306 | 0.6371 | 2.0456 | 1.0649 | 2 | 1 |
| WCM121_Z1_1 | 1 | 249150750 | 249212550 | −0.2457 | 0.4113 | NA | NA | NA | NA |
| WCM121_Z1_1 | 10 | 225958 | 294540 | −0.6799 | −0.677 | NA | NA | NA | NA |
| WCM121_Z1_1 | 10 | 294953 | 89624305 | −0.3988 | 0.08 | 2.0419 | 0.0721 | 2 | 0 |
| WCM121_Z1_1 | 10 | 89653806 | 97471746 | −0.7427 | −0.8891 | 1.0279 | 0.052 | 1 | 0 |
| WCM121_Z1_1 | 10 | 97515513 | 106581858 | 0.3725 | 1.4752 | 5.5972 | −0.0369 | 6 | 0 |
| WCM121_Z1_1 | 10 | 106602595 | 135381734 | −0.4011 | 0.0747 | 2.0924 | 0.0138 | 2 | 0 |
| WCM121_Z1_1 | 11 | 193112 | 58891926 | −0.1253 | 0.6471 | 2.0818 | 1.0503 | 2 | 1 |
| WCM121_Z1_1 | 11 | 58892177 | 108061213 | 0.0904 | 1.0294 | 2.0412 | 2.0412 | 2 | 2 |
| WCM121_Z1_1 | 11 | 108062894 | 113651182 | −0.0214 | 0.8367 | NA | NA | NA | NA |
| WCM121_Z1_1 | 11 | 113669963 | 114578268 | −0.6919 | −0.716 | NA | NA | NA | NA |
| WCM121_Z1_1 | 11 | 115040326 | 134856603 | −0.1389 | 0.6213 | 2.0054 | 1.0712 | 2 | 1 |
| WCM121_Z1_1 | 12 | 148675 | 38714888 | 0.2702 | 1.3193 | 2.8985 | 2.0922 | 3 | 2 |
| WCM121_Z1_1 | 12 | 39047733 | 133810751 | −0.1415 | 0.6165 | 2.0781 | 0.9882 | 2 | 1 |
| WCM121_Z1_1 | 13 | 19600515 | 37622053 | 0.0724 | 0.9991 | 2.0731 | 1.9244 | 2 | 2 |
| WCM121_Z1_1 | 13 | 37622734 | 88329447 | −0.4179 | 0.0357 | 2.0356 | 0.0145 | 2 | 0 |
| WCM121_Z1_1 | 13 | 88329761 | 115089411 | 0.0915 | 1.0313 | 2.1055 | 1.9822 | 2 | 2 |
| WCM121_Z1_1 | 14 | 19377726 | 39545199 | 0.0729 | 1 | 2.071 | 1.929 | 2 | 2 |
| WCM121_Z1_1 | 14 | 39555104 | 50052722 | −0.3386 | 0.2153 | NA | NA | NA | NA |
| WCM121_Z1_1 | 14 | 50053045 | 68301899 | 0.092 | 1.0321 | 2.117 | 1.9731 | 2 | 2 |
| WCM121_Z1_1 | 14 | 68331832 | 69117512 | −0.3841 | 0.1137 | NA | NA | NA | NA |
| WCM121_Z1_1 | 14 | 69256333 | 69989094 | −0.4545 | −0.0519 | 1.9546 | −0.0252 | 2 | 0 |
| WCM121_Z1_1 | 14 | 69991014 | 101198385 | −0.1474 | 0.6051 | 2.0405 | 1.0016 | 2 | 1 |
| WCM121_Z1_1 | 14 | 101200705 | 107283047 | 0.0831 | 1.0171 | 3.042 | 1.0059 | 3 | 1 |
| WCM121_Z1_1 | 15 | 20170019 | 35830632 | 0.5165 | 1.6858 | 4.5873 | 1.8469 | 5 | 2 |
| WCM121_Z1_1 | 15 | 35834693 | 102514200 | −0.1419 | 0.6156 | 2.0479 | 1.0166 | 2 | 1 |
| WCM121_Z1_1 | 16 | 67604 | 49193569 | 0.0799 | 1.0118 | 2.0402 | 1.9926 | 2 | 2 |
| WCM121_Z1_1 | 16 | 49313348 | 90233622 | −0.414 | 0.0447 | 2.0158 | 0.0472 | 2 | 0 |
| WCM121_Z1_1 | 17 | 131595 | 16326854 | −0.4139 | 0.045 | 1.0558 | 1.0076 | 1 | 1 |
| WCM121_Z1_1 | 17 | 16329476 | 20268662 | −0.1324 | 0.6336 | 2.0382 | 1.0646 | 2 | 1 |
| WCM121_Z1_1 | 17 | 20356346 | 33968991 | 0.077 | 1.0069 | 2.0808 | 1.9384 | 2 | 2 |
| WCM121_Z1_1 | 17 | 33977634 | 80914613 | −0.1537 | 0.5931 | 2.0402 | 0.9769 | 2 | 1 |
| WCM121_Z1_1 | 17 | 80915101 | 81083520 | −0.444 | −0.0264 | NA | NA | NA | NA |
| WCM121_Z1_1 | 18 | 48200 | 214889 | −0.1475 | 0.605 | NA | NA | NA | NA |
| WCM121_Z1_1 | 18 | 215504 | 42533055 | 0.0558 | 0.971 | 2.0203 | 1.9 | 2 | 2 |
| WCM121_Z1_1 | 18 | 42533280 | 78005215 | −0.1419 | 0.6156 | 2.0536 | 1.0107 | 2 | 1 |
| WCM121_Z1_1 | 19 | 281503 | 59083929 | 0.0537 | 0.9674 | 1.9853 | 1.9252 | 2 | 2 |
| WCM121_Z1_1 | 2 | 41612 | 286139 | 0.0848 | 1.02 | NA | NA | NA | NA |
| WCM121_Z1_1 | 2 | 287677 | 3464074 | 0.103 | 1.0504 | NA | NA | NA | NA |
| WCM121_Z1_1 | 2 | 3466632 | 37595798 | −0.1308 | 0.6367 | 2.0128 | 1.0968 | 2 | 1 |
| WCM121_Z1_1 | 2 | 37596853 | 44559625 | −0.7006 | −0.7445 | 0.943 | 0.2508 | 1 | 0 |
| WCM121_Z1_1 | 2 | 44565655 | 45715451 | −0.0423 | 0.7994 | NA | NA | NA | NA |
| WCM121_Z1_1 | 2 | 45773933 | 175213746 | 0.0766 | 1.0062 | 3.0458 | 0.9714 | 3 | 1 |
| WCM121_Z1_1 | 2 | 175215421 | 242946597 | 0.2767 | 1.3293 | 2.9022 | 2.1235 | 3 | 2 |
| WCM121_Z1_1 | 20 | 68373 | 62904866 | 0.0826 | 1.0162 | 2.0737 | 1.9716 | 2 | 2 |
| WCM121_Z1_1 | 21 | 9907280 | 39818748 | 0.0804 | 1.0125 | 2.0786 | 1.9563 | 2 | 2 |
| WCM121_Z1_1 | 21 | 39822803 | 42866328 | −0.3799 | 0.1234 | NA | NA | NA | NA |
| WCM121_Z1_1 | 21 | 42870082 | 48084281 | 0.085 | 1.0203 | 2.117 | 1.9397 | 2 | 2 |
| WCM121_Z1_1 | 22 | 17063431 | 28490068 | 0.0862 | 1.0223 | 2.113 | 1.9493 | 2 | 2 |
| WCM121_Z1_1 | 22 | 28492368 | 30421766 | −0.4684 | −0.0859 | 1.969 | −0.0846 | 2 | 0 |
| WCM121_Z1_1 | 22 | 30494916 | 46403039 | 0.0771 | 1.007 | 2.0911 | 1.9283 | 2 | 2 |
| WCM121_Z1_1 | 22 | 46447714 | 51219138 | −0.1609 | 0.5794 | 1.8967 | 1.0917 | 2 | 1 |
| WCM121_Z1_1 | 3 | 239398 | 119120818 | 0.0872 | 1.024 | 2.0677 | 1.9994 | 2 | 2 |
| WCM121_Z1_1 | 3 | 119121158 | 197444907 | 0.0814 | 1.0143 | 3.064 | 0.9759 | 3 | 1 |
| WCM121_Z1_1 | 3 | 197463575 | 197894713 | 0.0509 | 0.9626 | NA | NA | NA | NA |
| WCM121_Z1_1 | 4 | 59331 | 44724157 | −0.1309 | 0.6366 | 2.086 | 1.0233 | 2 | 1 |
| WCM121_Z1_1 | 4 | 46053573 | 52729629 | 0.5978 | 1.8005 | 4.9573 | 2.0095 | 5 | 2 |
| WCM121_Z1_1 | 4 | 52740498 | 52938243 | 0.1072 | 1.0575 | NA | NA | NA | NA |
| WCM121_Z1_1 | 4 | 52943087 | 148461073 | −0.1437 | 0.6122 | 2.0377 | 1.0194 | 2 | 1 |
| WCM121_Z1_1 | 4 | 148461553 | 167022017 | 0.23 | 1.2564 | 2.5058 | 2.2721 | 3 | 2 |
| WCM121_Z1_1 | 4 | 167656188 | 189013084 | −0.4013 | 0.0743 | 2.0945 | 0.0112 | 2 | 0 |
| WCM121_Z1_1 | 4 | 189014004 | 190426502 | −0.3693 | 0.1473 | NA | NA | NA | NA |
| WCM121_Z1_1 | 5 | 140595 | 2751474 | 0.0569 | 0.9728 | 2.1532 | 1.772 | 2 | 2 |
| WCM121_Z1_1 | 5 | 2752397 | 55218257 | −0.1228 | 0.6518 | 2.0169 | 1.1254 | 2 | 1 |
| WCM121_Z1_1 | 5 | 55237009 | 59901547 | −0.6794 | −0.6754 | NA | NA | NA | NA |
| WCM121_Z1_1 | 5 | 59934677 | 125885968 | −0.1271 | 0.6437 | 2.1686 | 0.956 | 2 | 1 |
| WCM121_Z1_1 | 5 | 125887715 | 180687768 | 0.0826 | 1.0162 | 2.0227 | 2.0227 | 2 | 2 |
| WCM121_Z1_1 | 6 | 203516 | 5771586 | 0.2182 | 1.2377 | 3.0873 | 1.6291 | 3 | 2 |
| WCM121_Z1_1 | 6 | 5999241 | 12296197 | −0.1042 | 0.6865 | 3.0747 | 0.1441 | 3 | 0 |
| WCM121_Z1_1 | 6 | 12933969 | 16307085 | 0.2004 | 1.2094 | NA | NA | NA | NA |
| WCM121_Z1_1 | 6 | 16326782 | 55929378 | 0.2803 | 1.3349 | 3.0105 | 2.0346 | 3 | 2 |
| WCM121_Z1_1 | 6 | 55933860 | 117630024 | 0.085 | 1.0203 | 2.1033 | 1.9533 | 2 | 2 |
| WCM121_Z1_1 | 6 | 117631388 | 159414899 | −0.146 | 0.6079 | 1.9831 | 1.0651 | 2 | 1 |
| WCM121_Z1_1 | 6 | 159420492 | 170893450 | −0.6981 | −0.7362 | 1.1947 | 0.0059 | 1 | 0 |
| WCM121_Z1_1 | 7 | 193254 | 158937416 | 0.4479 | 1.5866 | 4.0031 | 2.0038 | 4 | 2 |
| WCM121_Z1_1 | 8 | 190908 | 31012272 | −0.4181 | 0.0352 | 2.0327 | 0.0167 | 2 | 0 |
| WCM121_Z1_1 | 8 | 31015037 | 65291808 | −0.1467 | 0.6065 | 1.8768 | 1.1683 | 2 | 1 |
| WCM121_Z1_1 | 8 | 65489051 | 146279479 | 0.2904 | 1.3505 | 3.0178 | 2.0823 | 3 | 2 |
| WCM121_Z1_1 | 9 | 17075 | 3275569 | −0.1359 | 0.627 | 2.0387 | 1.0499 | 2 | 1 |
| WCM121_Z1_1 | 9 | 3277387 | 71534437 | 0.0979 | 1.042 | 2.0935 | 2.0247 | 2 | 2 |
| WCM121_Z1_1 | 9 | 71538291 | 141111414 | −0.1474 | 0.6052 | 1.9972 | 1.0453 | 2 | 1 |
| WCM121_Z1_1 | X | 2707709 | 65486401 | 0.0335 | 0.9326 | NA | NA | NA | NA |
| WCM121_Z1_1 | X | 65817849 | 68725908 | 3.9537 | 5.5976 | NA | NA | NA | NA |
| WCM121_Z1_1 | X | 68748854 | 70838300 | −0.4579 | −0.06 | NA | NA | NA | NA |
| WCM121_Z1_1 | X | 71131171 | 154509348 | 0.0344 | 0.9342 | NA | NA | NA | NA |
| WCM124 | 1 | 14930 | 249213964 | 0.0579 | NA | NA | NA | NA | NA |
| WCM124 | 10 | 226060 | 135438967 | 0.0294 | NA | NA | NA | NA | NA |
| WCM124 | 11 | 193096 | 65211940 | 0.0399 | NA | NA | NA | NA | NA |
| WCM124 | 11 | 65233857 | 65404406 | −0.4568 | NA | NA | NA | NA | NA |
| WCM124 | 11 | 65408667 | 134605954 | 0.0254 | NA | NA | NA | NA | NA |
| WCM124 | 12 | 148560 | 133810896 | 0.0305 | NA | NA | NA | NA | NA |
| WCM124 | 13 | 19041992 | 115089411 | −0.0095 | NA | NA | NA | NA | NA |
| WCM124 | 14 | 19110387 | 107283270 | 0.0305 | NA | NA | NA | NA | NA |
| WCM124 | 15 | 20083928 | 102516226 | 0.0489 | NA | NA | NA | NA | NA |
| WCM124 | 16 | 66921 | 90168701 | 0.012 | NA | NA | NA | NA | NA |
| WCM124 | 17 | 131595 | 81052551 | 0.0202 | NA | NA | NA | NA | NA |
| WCM124 | 18 | 47737 | 78005215 | 0.0216 | NA | NA | NA | NA | NA |
| WCM124 | 19 | 105080 | 59093645 | −0.0014 | NA | NA | NA | NA | NA |
| WCM124 | 2 | 41623 | 243037022 | 0.027 | NA | NA | NA | NA | NA |
| WCM124 | 20 | 68342 | 62934623 | 0.0433 | NA | NA | NA | NA | NA |
| WCM124 | 21 | 9590326 | 48084149 | −0.0157 | NA | NA | NA | NA | NA |
| WCM124 | 22 | 16159060 | 51219089 | 0.0456 | NA | NA | NA | NA | NA |
| WCM124 | 3 | 239443 | 197896800 | 0.0345 | NA | NA | NA | NA | NA |
| WCM124 | 4 | 53336 | 190878508 | 0.0022 | NA | NA | NA | NA | NA |
| WCM124 | 5 | 90684 | 180687800 | 0.0415 | NA | NA | NA | NA | NA |
| WCM124 | 6 | 203509 | 26108168 | 0.01 | NA | NA | NA | NA | NA |
| WCM124 | 6 | 26123684 | 26368372 | −0.9642 | NA | NA | NA | NA | NA |
| WCM124 | 6 | 26370569 | 41903720 | 0.0308 | NA | NA | NA | NA | NA |
| WCM124 | 6 | 41904276 | 42018312 | −1.0896 | NA | NA | NA | NA | NA |
| WCM124 | 6 | 42019208 | 170892848 | 0.0203 | NA | NA | NA | NA | NA |
| WCM124 | 7 | 193222 | 158937142 | 0.0379 | NA | NA | NA | NA | NA |
| WCM124 | 8 | 163470 | 146279527 | 0.0388 | NA | NA | NA | NA | NA |
| WCM124 | 9 | 14876 | 141107573 | 0.0523 | NA | NA | NA | NA | NA |
| WCM124 | X | 2700147 | 154509348 | 0.021 | NA | NA | NA | NA | NA |
| WCM124_X1_3 | 1 | 69640 | 145115810 | −0.1981 | 0.7647 | 2.2458 | 1.1522 | 2 | 1 |
| WCM124_X1_3 | 1 | 145209347 | 167298535 | 0.4452 | 1.7522 | 5.4341 | 1.3033 | 5 | 1 |
| WCM124_X1_3 | 1 | 167301765 | 167409963 | −0.8247 | −0.6169 | NA | NA | NA | NA |
| WCM124_X1_3 | 1 | 167487701 | 176564156 | 0.4247 | 1.7239 | 5.4997 | 1.1068 | 5 | 1 |
| WCM124_X1_3 | 1 | 176564418 | 177937094 | −0.7708 | −0.4633 | NA | NA | NA | NA |
| WCM124_X1_3 | 1 | 178062358 | 182026359 | 0.4069 | 1.6991 | 5.5612 | 0.9329 | 6 | 1 |
| WCM124_X1_3 | 1 | 182026496 | 185278606 | 1.0345 | 2.5136 | 10.0471 | 1.3739 | 10 | 1 |
| WCM124_X1_3 | 1 | 185703961 | 209602652 | 0.4915 | 1.8156 | 5.1689 | 1.8714 | 5 | 2 |
| WCM124_X1_3 | 1 | 209603818 | 210761383 | −0.7666 | −0.4519 | 1.3583 | 0.1039 | 1 | 0 |
| WCM124_X1_3 | 1 | 210796944 | 247020995 | 0.3865 | 1.6707 | 5.0821 | 1.2853 | 5 | 1 |
| WCM124_X1_3 | 1 | 247024353 | 248309356 | −0.8213 | −0.607 | 1.2238 | 0.0894 | 1 | 0 |
| WCM124_X1_3 | 1 | 248343317 | 248525792 | 0.4331 | 1.7355 | 5.323 | 1.337 | 5 | 1 |
| WCM124_X1_3 | 1 | 248551203 | 248551819 | −0.873 | −0.7646 | NA | NA | NA | NA |
| WCM124_X1_3 | 1 | 248569369 | 249212378 | 0.3799 | 1.6614 | NA | NA | NA | NA |
| WCM124_X1_3 | 10 | 225952 | 81932615 | −0.2164 | 0.7326 | 2.1047 | 1.2185 | 2 | 1 |
| WCM124_X1_3 | 10 | 82012599 | 82013316 | −1.3894 | −5.1259 | NA | NA | NA | NA |
| WCM124_X1_3 | 10 | 82033594 | 135440068 | −0.2112 | 0.7416 | 2.1668 | 1.1773 | 2 | 1 |
| WCM124_X1_3 | 11 | 193114 | 100211895 | 0.1546 | 1.3345 | 3.1101 | 1.9338 | 3 | 2 |
| WCM124_X1_3 | 11 | 100221537 | 123524497 | −0.2853 | 0.6085 | 3.0436 | 0.0057 | 3 | 0 |
| WCM124_X1_3 | 11 | 123596714 | 130784841 | −0.5474 | 0.084 | 2.1796 | −0.0597 | 2 | 0 |
| WCM124_X1_3 | 11 | 130785304 | 134856562 | −0.0191 | 1.0646 | NA | NA | NA | NA |
| WCM124_X1_3 | 12 | 148675 | 133810703 | 0.1452 | 1.3203 | 2.9232 | 2.0712 | 3 | 2 |
| WCM124_X1_3 | 13 | 19600515 | 19997239 | −0.5089 | 0.1675 | NA | NA | NA | NA |
| WCM124_X1_3 | 13 | 20012235 | 20413092 | 0.1498 | 1.3273 | NA | NA | NA | NA |
| WCM124_X1_3 | 13 | 20425532 | 21751155 | 0.8788 | 2.3208 | NA | NA | NA | NA |
| WCM124_X1_3 | 13 | 21949267 | 36167486 | 0.3067 | 1.5577 | 3.5468 | 2.3408 | 4 | 2 |
| WCM124_X1_3 | 13 | 36202341 | 108518940 | −0.5444 | 0.0907 | 2.056 | 0.0737 | 2 | 0 |
| WCM124_X1_3 | 13 | 108861007 | 115089411 | −0.2747 | 0.6279 | 3.0557 | 0.0348 | 3 | 0 |
| WCM124_X1_3 | 14 | 19377711 | 107283063 | −0.1934 | 0.7729 | 2.1648 | 1.2525 | 2 | 1 |
| WCM124_X1_3 | 15 | 20170150 | 102517639 | −0.2129 | 0.7387 | 2.1069 | 1.2304 | 2 | 1 |
| WCM124_X1_3 | 16 | 66921 | 50701948 | 0.0371 | 1.1539 | 2.2775 | 2.1726 | 2 | 2 |
| WCM124_X1_3 | 16 | 50703016 | 87799431 | −0.5514 | 0.0751 | 2.1188 | −0.012 | 2 | 0 |
| WCM124_X1_3 | 16 | 87868141 | 90162138 | 0.0705 | 1.206 | 2.3715 | 2.2426 | 2 | 2 |
| WCM124_X1_3 | 17 | 131595 | 81083455 | 0.0284 | 1.1401 | 2.2353 | 2.1727 | 2 | 2 |
| WCM124_X1_3 | 18 | 48206 | 357487 | 0.1171 | 1.2778 | NA | NA | NA | NA |
| WCM124_X1_3 | 18 | 480729 | 3742361 | 0.5275 | 1.8645 | 3.9804 | 3.3023 | 4 | 3 |
| WCM124_X1_3 | 18 | 3814138 | 78005194 | 0.1681 | 1.3548 | 3.1551 | 1.9601 | 3 | 2 |
| WCM124_X1_3 | 19 | 281498 | 59083953 | −0.0052 | 1.0869 | 2.1921 | 2.0562 | 2 | 2 |
| WCM124_X1_3 | 2 | 41614 | 116593753 | −0.2054 | 0.7519 | 2.1275 | 1.2405 | 2 | 1 |
| WCM124_X1_3 | 2 | 116594257 | 158400969 | −0.5277 | 0.127 | 2.1727 | 0.0113 | 2 | 0 |
| WCM124_X1_3 | 2 | 158406868 | 164468038 | −0.1773 | 0.8009 | 2.0467 | 1.4377 | 2 | 1 |
| WCM124_X1_3 | 2 | 164468200 | 185731121 | 0.1603 | 1.3431 | 3.8855 | 1.1883 | 4 | 1 |
| WCM124_X1_3 | 2 | 185798364 | 198675044 | 0.582 | 1.9375 | 3.8815 | 3.7794 | 4 | 4 |
| WCM124_X1_3 | 2 | 198948701 | 242946604 | 0.1656 | 1.3511 | 3.9085 | 1.1936 | 4 | 1 |
| WCM124_X1_3 | 20 | 68375 | 62904759 | −0.0028 | 1.0906 | 2.1671 | 2.0922 | 2 | 2 |
| WCM124_X1_3 | 21 | 9826225 | 48084214 | −0.2059 | 0.751 | 2.1027 | 1.2633 | 2 | 1 |
| WCM124_X1_3 | 22 | 16287459 | 51215124 | −0.2044 | 0.7536 | 2.1327 | 1.2392 | 2 | 1 |
| WCM124_X1_3 | 3 | 239349 | 54880447 | −0.1897 | 0.7793 | 2.1736 | 1.259 | 2 | 1 |
| WCM124_X1_3 | 3 | 54905607 | 142756978 | 0.1506 | 1.3286 | 3.8549 | 1.1681 | 4 | 1 |
| WCM124_X1_3 | 3 | 142757744 | 170943409 | −0.2674 | 0.6412 | 1.6587 | 1.4605 | 2 | 1 |
| WCM124_X1_3 | 3 | 170946002 | 197896720 | 0.2392 | 1.46 | 4.3244 | 1.1778 | 4 | 1 |
| WCM124_X1_3 | 4 | 59327 | 135815 | −0.548 | 0.0827 | NA | NA | NA | NA |
| WCM124_X1_3 | 4 | 154907 | 115898402 | −0.2291 | 0.7101 | 2.0693 | 1.2025 | 2 | 1 |
| WCM124_X1_3 | 4 | 115997246 | 138453414 | −0.5202 | 0.1431 | 1.922 | 0.2866 | 2 | 0 |
| WCM124_X1_3 | 4 | 139100420 | 183270997 | −0.2418 | 0.6873 | 2.0889 | 1.1317 | 2 | 1 |
| WCM124_X1_3 | 4 | 183370160 | 190426502 | −0.5168 | 0.1505 | 1.8652 | 0.3546 | 2 | 0 |
| WCM124_X1_3 | 5 | 140477 | 45695899 | 0.4989 | 1.8257 | 4.1084 | 2.9811 | 4 | 3 |
| WCM124_X1_3 | 5 | 49698142 | 85915201 | −0.0966 | 0.9377 | 2.754 | 1.077 | 3 | 1 |
| WCM124_X1_3 | 5 | 85916329 | 109103398 | −0.3766 | 0.4364 | 2.8106 | −0.1042 | 3 | 0 |
| WCM124_X1_3 | 5 | 109110590 | 166802458 | −0.0783 | 0.9681 | 2.7344 | 1.1781 | 3 | 1 |
| WCM124_X1_3 | 5 | 167182180 | 180687808 | 0.1596 | 1.342 | 2.615 | 2.4551 | 3 | 2 |
| WCM124_X1_3 | 6 | 203515 | 79912064 | −0.0282 | 1.0499 | 2.1105 | 2.0302 | 2 | 2 |
| WCM124_X1_3 | 6 | 79913340 | 88773931 | −0.5345 | 0.1122 | 2.1918 | −0.03 | 2 | 0 |
| WCM124_X1_3 | 6 | 88853635 | 123384862 | −0.0345 | 1.0398 | 2.0858 | 2.0259 | 2 | 2 |
| WCM124_X1_3 | 6 | 123539855 | 128403745 | −0.4873 | 0.2131 | NA | NA | NA | NA |
| WCM124_X1_3 | 6 | 128404866 | 170893450 | −0.0228 | 1.0586 | 2.1098 | 2.0561 | 2 | 2 |
| WCM124_X1_3 | 7 | 193222 | 38765896 | 0.0483 | 1.1715 | 3.2994 | 1.2054 | 3 | 1 |
| WCM124_X1_3 | 7 | 38766569 | 45956969 | 0.2212 | 1.4336 | 2.9954 | 2.4072 | 3 | 2 |
| WCM124_X1_3 | 7 | 45960865 | 65842221 | 0.0668 | 1.2003 | 3.4298 | 1.166 | 3 | 1 |
| WCM124_X1_3 | 7 | 65842545 | 78131080 | −0.1885 | 0.7814 | 3.1787 | 0.2589 | 3 | 0 |
| WCM124_X1_3 | 7 | 78150885 | 158937018 | 0.0514 | 1.1763 | 3.2702 | 1.2496 | 3 | 1 |
| WCM124_X1_3 | 8 | 190907 | 81582832 | 0.3661 | 1.6421 | 4.2198 | 2.0228 | 4 | 2 |
| WCM124_X1_3 | 8 | 81605315 | 94811977 | −0.255 | 0.6636 | 2.4268 | 0.7413 | 2 | 1 |
| WCM124_X1_3 | 8 | 94815859 | 109491237 | 0.4152 | 1.7107 | 4.4735 | 2.0732 | 4 | 2 |
| WCM124_X1_3 | 8 | 109498828 | 114388973 | −0.5146 | 0.1551 | NA | NA | NA | NA |
| WCM124_X1_3 | 8 | 114448950 | 132051970 | 0.4148 | 1.7102 | 3.581 | 2.9629 | 4 | 3 |
| WCM124_X1_3 | 8 | 132052217 | 134538157 | −0.4428 | 0.305 | NA | NA | NA | NA |
| WCM124_X1_3 | 8 | 135490959 | 146279424 | 0.5091 | 1.8396 | 4.3602 | 2.7979 | 4 | 3 |
| WCM124_X1_3 | 9 | 17079 | 17330665 | −0.5536 | 0.0703 | 1.9581 | 0.1418 | 2 | 0 |
| WCM124_X1_3 | 9 | 17332618 | 24543733 | 0.1044 | 1.2583 | 3.0811 | 1.7031 | 3 | 2 |
| WCM124_X1_3 | 9 | 24544240 | 90582224 | 0.3881 | 1.673 | 4.2462 | 2.1314 | 4 | 2 |
| WCM124_X1_3 | 9 | 90582449 | 141111414 | 0.2256 | 1.4401 | 4.3238 | 1.103 | 4 | 1 |
| WCM124_X1_3 | X | 2707694 | 57936541 | −0.0736 | 0.9759 | NA | NA | NA | NA |
| WCM124_X1_3 | X | 62898360 | 63445548 | 3.6159 | 5.3407 | NA | NA | NA | NA |
| WCM124_X1_3 | X | 63488455 | 63615223 | 0.092 | 1.2392 | NA | NA | NA | NA |
| WCM124_X1_3 | X | 64137685 | 69397492 | 3.2063 | 4.9161 | NA | NA | NA | NA |
| WCM124_X1_3 | X | 69419191 | 83599274 | −0.1259 | 0.8887 | NA | NA | NA | NA |
| WCM124_X1_3 | X | 83616567 | 89177098 | −1.2638 | −2.7663 | NA | NA | NA | NA |
| WCM124_X1_3 | X | 89177431 | 154509348 | −0.0992 | 0.9334 | NA | NA | NA | NA |
| WCM12_Z10_1 | 1 | 721450 | 3775357 | −0.1157 | −0.0915 | 0.9647 | 0.9124 | 1 | 1 |
| WCM12_Z10_1 | 1 | 3776990 | 29863127 | −0.7405 | −0.9411 | 1.0558 | −0.0141 | 1 | 0 |
| WCM12_Z10_1 | 1 | 31186459 | 145748533 | −0.0467 | −0.0046 | 1.0043 | 0.9894 | 1 | 1 |
| WCM12_Z10_1 | 1 | 146398489 | 166039898 | 0.4347 | 0.576 | 1.992 | 0.9894 | 2 | 1 |
| WCM12_Z10_1 | 1 | 166124000 | 185183757 | 1.0716 | 1.2968 | 3.8859 | 1.0276 | 4 | 1 |
| WCM12_Z10_1 | 1 | 185191034 | 249212344 | 0.4583 | 0.6035 | 2.0679 | 0.9709 | 2 | 1 |
| WCM12_Z10_1 | 10 | 225979 | 8006038 | −0.0385 | 0.0056 | 1.0377 | 0.97 | 1 | 1 |
| WCM12_Z10_1 | 10 | 8006262 | 8007471 | −1.677 | −2.7108 | NA | NA | NA | NA |
| WCM12_Z10_1 | 10 | 8051281 | 58118353 | −0.0613 | −0.0229 | 1.0065 | 0.962 | 1 | 1 |
| WCM12_Z10_1 | 10 | 58118630 | 104309801 | −0.6833 | −0.8574 | 1.1061 | −0.0022 | 1 | 0 |
| WCM12_Z10_1 | 10 | 104352421 | 125426022 | −0.0513 | −0.0104 | 0.9928 | 0.9928 | 1 | 1 |
| WCM12_Z10_1 | 10 | 125426412 | 135381660 | −0.6597 | −0.8231 | 1.1718 | −0.0413 | 1 | 0 |
| WCM12_Z10_1 | 11 | 193112 | 95826599 | −0.0585 | −0.0194 | 1.0435 | 0.9297 | 1 | 1 |
| WCM12_Z10_1 | 11 | 96074691 | 109963934 | −0.7767 | −0.9951 | 0.9527 | 0.0507 | 1 | 0 |
| WCM12_Z10_1 | 11 | 110001831 | 113860464 | −0.1591 | −0.1467 | NA | NA | NA | NA |
| WCM12_Z10_1 | 11 | 113934214 | 117152688 | −0.7062 | −0.8908 | 1.0508 | 0.0278 | 1 | 0 |
| WCM12_Z10_1 | 11 | 117153121 | 134856592 | −0.0922 | −0.0618 | 0.9726 | 0.9436 | 1 | 1 |
| WCM12_Z10_1 | 12 | 148644 | 114839669 | −0.0362 | 0.0085 | 1.0159 | 0.9958 | 1 | 1 |
| WCM12_Z10_1 | 12 | 114841600 | 118852193 | −0.6874 | −0.8632 | NA | NA | NA | NA |
| WCM12_Z10_1 | 12 | 119419695 | 133810703 | 0.0086 | 0.0642 | 1.0571 | 1.0338 | 1 | 1 |
| WCM12_Z10_1 | 13 | 19600515 | 47470924 | −0.0739 | −0.0387 | 0.9919 | 0.9552 | 1 | 1 |
| WCM12_Z10_1 | 13 | 48517532 | 92560249 | −0.7086 | −0.8942 | 1.1054 | −0.0293 | 1 | 0 |
| WCM12_Z10_1 | 13 | 92797222 | 115089411 | −0.0127 | 0.0378 | 1.0495 | 1.0036 | 1 | 1 |
| WCM12_Z10_1 | 14 | 19377701 | 65878993 | −0.0324 | 0.0133 | 1.0256 | 0.9929 | 1 | 1 |
| WCM12_Z10_1 | 14 | 65937853 | 107283034 | −0.7459 | −0.9492 | 1.044 | −0.0081 | 1 | 0 |
| WCM12_Z10_1 | 15 | 20170150 | 36905707 | −0.1447 | −0.1283 | 1.085 | 0.7448 | 1 | 1 |
| WCM12_Z10_1 | 15 | 36906129 | 66190347 | −0.7526 | −0.9592 | 1.0265 | 0.0022 | 1 | 0 |
| WCM12_Z10_1 | 15 | 66191192 | 102517660 | −0.0476 | −0.0058 | 1.0035 | 0.9885 | 1 | 1 |
| WCM12_Z10_1 | 16 | 67604 | 74385987 | −0.0857 | −0.0536 | 1.0436 | 0.8834 | 1 | 1 |
| WCM12_Z10_1 | 16 | 74394547 | 90233626 | −0.7862 | −1.0093 | 0.9889 | 0.0047 | 1 | 0 |
| WCM12_Z10_1 | 17 | 131595 | 13503897 | −0.7724 | −0.9887 | 1.0181 | −0.0102 | 1 | 0 |
| WCM12_Z10_1 | 17 | 13504330 | 81083561 | 0.016 | 0.0734 | 1.0639 | 1.0404 | 1 | 1 |
| WCM12_Z10_1 | 18 | 48204 | 78005164 | −0.0473 | −0.0054 | 1.0037 | 0.9888 | 1 | 1 |
| WCM12_Z10_1 | 19 | 281430 | 59083953 | −0.0694 | −0.0331 | 0.9859 | 0.9687 | 1 | 1 |
| WCM12_Z10_1 | 2 | 41615 | 63849759 | 0.0039 | 0.0584 | 1.0543 | 1.0284 | 1 | 1 |
| WCM12_Z10_1 | 2 | 64069326 | 74273508 | −0.6523 | −0.8126 | 1.0036 | 0.1351 | 1 | 0 |
| WCM12_Z10_1 | 2 | 74273887 | 119600513 | −0.0775 | −0.0432 | 0.9961 | 0.9448 | 1 | 1 |
| WCM12_Z10_1 | 2 | 119600793 | 232952369 | 0.3622 | 0.4909 | 1.8622 | 0.9485 | 2 | 1 |
| WCM12_Z10_1 | 2 | 232975953 | 242946616 | −0.1724 | −0.1637 | 1.8058 | −0.0203 | 2 | 0 |
| WCM12_Z10_1 | 20 | 68363 | 62904835 | −0.0457 | −0.0034 | 1.0089 | 0.9864 | 1 | 1 |
| WCM12_Z10_1 | 21 | 9826207 | 48084214 | −0.132 | −0.1121 | 0.9452 | 0.9053 | 1 | 1 |
| WCM12_Z10_1 | 22 | 17063530 | 51219138 | −0.1094 | −0.0835 | 0.9509 | 0.9366 | 1 | 1 |
| WCM12_Z10_1 | 3 | 239472 | 37778406 | −0.2507 | −0.2646 | 0.9417 | 0.7231 | 1 | 1 |
| WCM12_Z10_1 | 3 | 37783236 | 197894720 | −0.0322 | 0.0135 | 1.0195 | 0.9993 | 1 | 1 |
| WCM12_Z10_1 | 4 | 59361 | 36069702 | −0.8038 | −1.0359 | 0.9732 | 0.0023 | 1 | 0 |
| WCM12_Z10_1 | 4 | 36069899 | 169013864 | −0.0949 | −0.0651 | 0.9619 | 0.9498 | 1 | 1 |
| WCM12_Z10_1 | 4 | 169048901 | 190396288 | 0.3853 | 0.5181 | 2.0359 | 0.8282 | 2 | 1 |
| WCM12_Z10_1 | 5 | 140532 | 6737737 | 0.2472 | 0.3545 | 1.6194 | 0.9377 | 2 | 1 |
| WCM12_Z10_1 | 5 | 6738823 | 180486841 | −0.0758 | −0.0411 | 0.9805 | 0.9633 | 1 | 1 |
| WCM12_Z10_1 | 5 | 180551500 | 180687769 | 0.0861 | 0.1597 | NA | NA | NA | NA |
| WCM12_Z10_1 | 6 | 203542 | 6169570 | 0.0499 | 0.1152 | 1.1471 | 1.0191 | 1 | 1 |
| WCM12_Z10_1 | 6 | 6174866 | 30139699 | 0.2531 | 0.3616 | 1.5181 | 1.0515 | 2 | 1 |
| WCM12_Z10_1 | 6 | 30140061 | 101312005 | −0.0258 | 0.0215 | 1.034 | 0.996 | 1 | 1 |
| WCM12_Z10_1 | 6 | 101315874 | 170893409 | −0.7557 | −0.9637 | 1.0162 | 0.0093 | 1 | 0 |
| WCM12_Z10_1 | 7 | 193253 | 6776956 | 0.3953 | 0.5298 | 1.8841 | 1.0034 | 2 | 1 |
| WCM12_Z10_1 | 7 | 6777171 | 92462598 | −0.0407 | 0.0029 | 1.0158 | 0.9883 | 1 | 1 |
| WCM12_Z10_1 | 7 | 92730682 | 94827762 | −0.7513 | −0.9571 | NA | NA | NA | NA |
| WCM12_Z10_1 | 7 | 94855320 | 158937074 | −0.031 | 0.015 | 1.0231 | 0.9979 | 1 | 1 |
| WCM12_Z10_1 | 8 | 190908 | 48511513 | −0.7616 | −0.9725 | 1.0163 | 0.003 | 1 | 0 |
| WCM12_Z10_1 | 8 | 48566697 | 128750683 | 0.0054 | 0.0603 | 1.0634 | 1.022 | 1 | 1 |
| WCM12_Z10_1 | 8 | 128750852 | 146279513 | 0.3252 | 0.4472 | 1.8861 | 0.8406 | 2 | 1 |
| WCM12_Z10_1 | 9 | 116832 | 141111414 | −0.0046 | 0.0479 | 1.044 | 1.0235 | 1 | 1 |
| WCM12_Z10_1 | X | 2707682 | 122840795 | −0.0769 | −0.0426 | NA | NA | NA | NA |
| WCM12_Z10_1 | X | 122866861 | 154509348 | 0.7214 | 0.9057 | NA | NA | NA | NA |
| WCM131 | 1 | 14930 | 249214018 | 0.0196 | NA | NA | NA | NA | NA |
| WCM131 | 10 | 225995 | 135439027 | 0.0181 | NA | NA | NA | NA | NA |
| WCM131 | 11 | 193096 | 134605916 | 0.01 | NA | NA | NA | NA | NA |
| WCM131 | 12 | 148480 | 133810868 | 0.0064 | NA | NA | NA | NA | NA |
| WCM131 | 13 | 19041965 | 115089411 | −0.0043 | NA | NA | NA | NA | NA |
| WCM131 | 14 | 19110401 | 107283269 | 0.002 | NA | NA | NA | NA | NA |
| WCM131 | 15 | 20083896 | 102506248 | 0.025 | NA | NA | NA | NA | NA |
| WCM131 | 16 | 66904 | 90168693 | −0.0033 | NA | NA | NA | NA | NA |
| WCM131 | 17 | 131595 | 81187930 | −0.0013 | NA | NA | NA | NA | NA |
| WCM131 | 18 | 47799 | 78005199 | 0.0076 | NA | NA | NA | NA | NA |
| WCM131 | 19 | 105069 | 59093645 | −0.0136 | NA | NA | NA | NA | NA |
| WCM131 | 2 | 41720 | 243037022 | 0.0043 | NA | NA | NA | NA | NA |
| WCM131 | 20 | 68446 | 62934592 | 0.0232 | NA | NA | NA | NA | NA |
| WCM131 | 21 | 9590314 | 48084198 | 0.0136 | NA | NA | NA | NA | NA |
| WCM131 | 22 | 16159021 | 51223730 | 0.0331 | NA | NA | NA | NA | NA |
| WCM131 | 3 | 239472 | 197896735 | 0.009 | NA | NA | NA | NA | NA |
| WCM131 | 4 | 53342 | 190904471 | 0.0055 | NA | NA | NA | NA | NA |
| WCM131 | 5 | 90576 | 180687847 | 0.0069 | NA | NA | NA | NA | NA |
| WCM131 | 6 | 203515 | 170892918 | −0.0032 | NA | NA | NA | NA | NA |
| WCM131 | 7 | 193222 | 158937135 | 0.0065 | NA | NA | NA | NA | NA |
| WCM131 | 8 | 163491 | 146279527 | 0.0085 | NA | NA | NA | NA | NA |
| WCM131 | 9 | 14893 | 141107526 | 0.0131 | NA | NA | NA | NA | NA |
| WCM131 | X | 2700145 | 154493598 | −0.1115 | NA | NA | NA | NA | NA |
| WCM131_Z1_1 | 1 | 721450 | 24202102 | −0.597 | NA | NA | NA | NA | NA |
| WCM131_Z1_1 | 1 | 24255586 | 26370001 | 0.1169 | NA | NA | NA | NA | NA |
| WCM131_Z1_1 | 1 | 26370907 | 116228092 | −0.5954 | NA | NA | NA | NA | NA |
| WCM131_Z1_1 | 1 | 116233977 | 145747117 | 0.0303 | NA | NA | NA | NA | NA |
| WCM131_Z1_1 | 1 | 145748513 | 155238115 | 0.767 | NA | NA | NA | NA | NA |
| WCM131_Z1_1 | 1 | 155238505 | 207224048 | 0.4195 | NA | NA | NA | NA | NA |
| WCM131_Z1_1 | 1 | 207224359 | 249212348 | 0.0019 | NA | NA | NA | NA | NA |
| WCM131_Z1_1 | 10 | 226016 | 3822362 | 0.0027 | NA | NA | NA | NA | NA |
| WCM131_Z1_1 | 10 | 3823897 | 14563967 | 0.4371 | NA | NA | NA | NA | NA |
| WCM131_Z1_1 | 10 | 14572451 | 134421427 | 0.0218 | NA | NA | NA | NA | NA |
| WCM131_Z1_1 | 10 | 134459468 | 135170161 | 0.4274 | NA | NA | NA | NA | NA |
| WCM131_Z1_1 | 10 | 135170689 | 135440068 | −0.1036 | NA | NA | NA | NA | NA |
| WCM131_Z1_1 | 11 | 193097 | 544740 | 0.0932 | NA | NA | NA | NA | NA |
| WCM131_Z1_1 | 11 | 549959 | 831594 | −0.568 | NA | NA | NA | NA | NA |
| WCM131_Z1_1 | 11 | 836266 | 1075919 | −0.0805 | NA | NA | NA | NA | NA |
| WCM131_Z1_1 | 11 | 1077710 | 1580257 | 0.4471 | NA | NA | NA | NA | NA |
| WCM131_Z1_1 | 11 | 1585102 | 9466641 | 0.0546 | NA | NA | NA | NA | NA |
| WCM131_Z1_1 | 11 | 9492941 | 10830000 | −0.5572 | NA | NA | NA | NA | NA |
| WCM131_Z1_1 | 11 | 10874480 | 30032622 | 0.0254 | NA | NA | NA | NA | NA |
| WCM131_Z1_1 | 11 | 30032899 | 48238549 | −0.5891 | NA | NA | NA | NA | NA |
| WCM131_Z1_1 | 11 | 48238633 | 48997107 | −0.5697 | NA | NA | NA | NA | NA |
| WCM131_Z1_1 | 11 | 48997334 | 50003964 | −0.7959 | NA | NA | NA | NA | NA |
| WCM131_Z1_1 | 11 | 51411709 | 62462042 | −0.007 | NA | NA | NA | NA | NA |
| WCM131_Z1_1 | 11 | 62470003 | 62656161 | −0.5806 | NA | NA | NA | NA | NA |
| WCM131_Z1_1 | 11 | 62677212 | 82752546 | 0.0146 | NA | NA | NA | NA | NA |
| WCM131_Z1_1 | 11 | 82868529 | 117060072 | −0.5921 | NA | NA | NA | NA | NA |
| WCM131_Z1_1 | 11 | 117060706 | 117258120 | −1.6734 | NA | NA | NA | NA | NA |
| WCM131_Z1_1 | 11 | 117261523 | 134856534 | −0.5534 | NA | NA | NA | NA | NA |
| WCM131_Z1_1 | 12 | 148674 | 1225133 | 0.7721 | NA | NA | NA | NA | NA |
| WCM131_Z1_1 | 12 | 1250923 | 1863601 | −0.4715 | NA | NA | NA | NA | NA |
| WCM131_Z1_1 | 12 | 1882119 | 6101103 | 0.7833 | NA | NA | NA | NA | NA |
| WCM131_Z1_1 | 12 | 6103308 | 6761878 | 1.0964 | NA | NA | NA | NA | NA |
| WCM131_Z1_1 | 12 | 6762126 | 9907272 | 0.7262 | NA | NA | NA | NA | NA |
| WCM131_Z1_1 | 12 | 9907821 | 27235396 | 0.4236 | NA | NA | NA | NA | NA |
| WCM131_Z1_1 | 12 | 27450665 | 112681213 | 0.0098 | NA | NA | NA | NA | NA |
| WCM131_Z1_1 | 12 | 112681690 | 115120981 | −0.4301 | NA | NA | NA | NA | NA |
| WCM131_Z1_1 | 12 | 115801927 | 123345258 | 0.0242 | NA | NA | NA | NA | NA |
| WCM131_Z1_1 | 12 | 123345509 | 123751791 | −0.5677 | NA | NA | NA | NA | NA |
| WCM131_Z1_1 | 12 | 123780572 | 133810892 | 0.0334 | NA | NA | NA | NA | NA |
| WCM131_Z1_1 | 13 | 19600475 | 40174999 | 0.0329 | NA | NA | NA | NA | NA |
| WCM131_Z1_1 | 13 | 40175313 | 74536134 | −0.5534 | NA | NA | NA | NA | NA |
| WCM131_Z1_1 | 13 | 74569131 | 115089411 | 0.0157 | NA | NA | NA | NA | NA |
| WCM131_Z1_1 | 14 | 19377718 | 67940191 | −0.0232 | NA | NA | NA | NA | NA |
| WCM131_Z1_1 | 14 | 67940419 | 68331821 | 0.4085 | NA | NA | NA | NA | NA |
| WCM131_Z1_1 | 14 | 68353820 | 107283055 | −0.0757 | NA | NA | NA | NA | NA |
| WCM131_Z1_1 | 15 | 20170149 | 41625188 | 0.0147 | NA | NA | NA | NA | NA |
| WCM131_Z1_1 | 15 | 41634628 | 42284971 | −0.6115 | NA | NA | NA | NA | NA |
| WCM131_Z1_1 | 15 | 42287659 | 102506284 | −0.0021 | NA | NA | NA | NA | NA |
| WCM131_Z1_1 | 16 | 67604 | 4564091 | −0.6007 | NA | NA | NA | NA | NA |
| WCM131_Z1_1 | 16 | 4606515 | 7657307 | −0.0329 | NA | NA | NA | NA | NA |
| WCM131_Z1_1 | 16 | 7680646 | 10788727 | 0.3699 | NA | NA | NA | NA | NA |
| WCM131_Z1_1 | 16 | 10837900 | 23722339 | −0.0645 | NA | NA | NA | NA | NA |
| WCM131_Z1_1 | 16 | 23723376 | 27078284 | 0.485 | NA | NA | NA | NA | NA |
| WCM131_Z1_1 | 16 | 27078441 | 70211290 | −0.0234 | NA | NA | NA | NA | NA |
| WCM131_Z1_1 | 16 | 70255159 | 70589097 | −0.6648 | NA | NA | NA | NA | NA |
| WCM131_Z1_1 | 16 | 70590158 | 79245678 | −0.0449 | NA | NA | NA | NA | NA |
| WCM131_Z1_1 | 16 | 79245961 | 90161578 | −0.5959 | NA | NA | NA | NA | NA |
| WCM131_Z1_1 | 17 | 131595 | 1198881 | 0.0273 | NA | NA | NA | NA | NA |
| WCM131_Z1_1 | 17 | 1201548 | 1782313 | −0.5637 | NA | NA | NA | NA | NA |
| WCM131_Z1_1 | 17 | 1782634 | 9843458 | 0.0148 | NA | NA | NA | NA | NA |
| WCM131_Z1_1 | 17 | 9846447 | 11166785 | 0.3998 | NA | NA | NA | NA | NA |
| WCM131_Z1_1 | 17 | 11282745 | 58023929 | 0.0023 | NA | NA | NA | NA | NA |
| WCM131_Z1_1 | 17 | 58030386 | 74475409 | 0.4161 | NA | NA | NA | NA | NA |
| WCM131_Z1_1 | 17 | 74475975 | 81083507 | 0.0255 | NA | NA | NA | NA | NA |
| WCM131_Z1_1 | 18 | 49008 | 78005215 | 0.0134 | NA | NA | NA | NA | NA |
| WCM131_Z1_1 | 19 | 281439 | 12461928 | −0.0051 | NA | NA | NA | NA | NA |
| WCM131_Z1_1 | 19 | 12462873 | 12924269 | −0.7021 | NA | NA | NA | NA | NA |
| WCM131_Z1_1 | 19 | 12936591 | 51631714 | −0.0081 | NA | NA | NA | NA | NA |
| WCM131_Z1_1 | 19 | 51633231 | 59083988 | 0.7353 | NA | NA | NA | NA | NA |
| WCM131_Z1_1 | 2 | 41623 | 43015693 | 0.003 | NA | NA | NA | NA | NA |
| WCM131_Z1_1 | 2 | 43019627 | 43547589 | −1.6013 | NA | NA | NA | NA | NA |
| WCM131_Z1_1 | 2 | 43571344 | 72960206 | −0.018 | NA | NA | NA | NA | NA |
| WCM131_Z1_1 | 2 | 72968457 | 85778171 | 0.3523 | NA | NA | NA | NA | NA |
| WCM131_Z1_1 | 2 | 85778728 | 144966233 | 0.0042 | NA | NA | NA | NA | NA |
| WCM131_Z1_1 | 2 | 145146448 | 155102330 | 0.3469 | NA | NA | NA | NA | NA |
| WCM131_Z1_1 | 2 | 155115634 | 160884867 | −0.0495 | NA | NA | NA | NA | NA |
| WCM131_Z1_1 | 2 | 160885457 | 203500084 | 0.3754 | NA | NA | NA | NA | NA |
| WCM131_Z1_1 | 2 | 203500509 | 204291988 | −0.6456 | NA | NA | NA | NA | NA |
| WCM131_Z1_1 | 2 | 204302883 | 212295757 | 0.3791 | NA | NA | NA | NA | NA |
| WCM131_Z1_1 | 2 | 212426660 | 213290991 | −0.5992 | NA | NA | NA | NA | NA |
| WCM131_Z1_1 | 2 | 213403201 | 233349972 | 0.0052 | NA | NA | NA | NA | NA |
| WCM131_Z1_1 | 2 | 233351271 | 241938464 | −0.6184 | NA | NA | NA | NA | NA |
| WCM131_Z1_1 | 2 | 241969873 | 242151579 | 0.1397 | NA | NA | NA | NA | NA |
| WCM131_Z1_1 | 2 | 242152031 | 242946597 | −0.5753 | NA | NA | NA | NA | NA |
| WCM131_Z1_1 | 20 | 68363 | 3520963 | −0.5973 | NA | NA | NA | NA | NA |
| WCM131_Z1_1 | 20 | 3526441 | 3785506 | 0.0256 | NA | NA | NA | NA | NA |
| WCM131_Z1_1 | 20 | 3802885 | 8755241 | −0.6408 | NA | NA | NA | NA | NA |
| WCM131_Z1_1 | 20 | 8769158 | 62853302 | 0.0174 | NA | NA | NA | NA | NA |
| WCM131_Z1_1 | 20 | 62854504 | 62904847 | −0.4721 | NA | NA | NA | NA | NA |
| WCM131_Z1_1 | 21 | 9826207 | 47613559 | 0.0197 | NA | NA | NA | NA | NA |
| WCM131_Z1_1 | 21 | 47614553 | 47981595 | −0.6693 | NA | NA | NA | NA | NA |
| WCM131_Z1_1 | 21 | 47983779 | 48084214 | 0.1486 | NA | NA | NA | NA | NA |
| WCM131_Z1_1 | 22 | 16287495 | 50649328 | −4.00E−04 | NA | NA | NA | NA | NA |
| WCM131_Z1_1 | 22 | 50651066 | 50885266 | −0.5529 | NA | NA | NA | NA | NA |
| WCM131_Z1_1 | 22 | 50885604 | 51215123 | 0.0295 | NA | NA | NA | NA | NA |
| WCM131_Z1_1 | 3 | 239431 | 44283843 | 0.0117 | NA | NA | NA | NA | NA |
| WCM131_Z1_1 | 3 | 44284584 | 58923421 | −0.5944 | NA | NA | NA | NA | NA |
| WCM131_Z1_1 | 3 | 59032925 | 71027103 | 0.0399 | NA | NA | NA | NA | NA |
| WCM131_Z1_1 | 3 | 71037163 | 71247364 | −1.5966 | NA | NA | NA | NA | NA |
| WCM131_Z1_1 | 3 | 71591198 | 197894719 | 0.0068 | NA | NA | NA | NA | NA |
| WCM131_Z1_1 | 4 | 59367 | 151186883 | 0.0128 | NA | NA | NA | NA | NA |
| WCM131_Z1_1 | 4 | 151199013 | 151518249 | −0.4792 | NA | NA | NA | NA | NA |
| WCM131_Z1_1 | 4 | 151520247 | 151765916 | −1.7718 | NA | NA | NA | NA | NA |
| WCM131_Z1_1 | 4 | 151769983 | 152201053 | −0.814 | NA | NA | NA | NA | NA |
| WCM131_Z1_1 | 4 | 152203541 | 155219225 | 0.004 | NA | NA | NA | NA | NA |
| WCM131_Z1_1 | 4 | 155219382 | 190426502 | 0.4067 | NA | NA | NA | NA | NA |
| WCM131_Z1_1 | 5 | 140636 | 1880865 | 0.6298 | NA | NA | NA | NA | NA |
| WCM131_Z1_1 | 5 | 2747699 | 51376661 | 0.0086 | NA | NA | NA | NA | NA |
| WCM131_Z1_1 | 5 | 52096412 | 54721132 | −0.6328 | NA | NA | NA | NA | NA |
| WCM131_Z1_1 | 5 | 54721781 | 78324352 | −0.0131 | NA | NA | NA | NA | NA |
| WCM131_Z1_1 | 5 | 78325809 | 134670827 | −0.3184 | NA | NA | NA | NA | NA |
| WCM131_Z1_1 | 5 | 134673651 | 180687803 | 0.0053 | NA | NA | NA | NA | NA |
| WCM131_Z1_1 | 6 | 203466 | 43188940 | 0.0118 | NA | NA | NA | NA | NA |
| WCM131_Z1_1 | 6 | 43189484 | 70778316 | 0.0131 | NA | NA | NA | NA | NA |
| WCM131_Z1_1 | 6 | 70812090 | 137112891 | −0.011 | NA | NA | NA | NA | NA |
| WCM131_Z1_1 | 6 | 137113228 | 170893409 | 0.4307 | NA | NA | NA | NA | NA |
| WCM131_Z1_1 | 7 | 193222 | 102825981 | 0.0151 | NA | NA | NA | NA | NA |
| WCM131_Z1_1 | 7 | 102850754 | 104844175 | 0.4243 | NA | NA | NA | NA | NA |
| WCM131_Z1_1 | 7 | 104844543 | 133827853 | 0.0017 | NA | NA | NA | NA | NA |
| WCM131_Z1_1 | 7 | 133842826 | 133991526 | 0.3116 | NA | NA | NA | NA | NA |
| WCM131_Z1_1 | 7 | 133994206 | 149417995 | 0.4742 | NA | NA | NA | NA | NA |
| WCM131_Z1_1 | 7 | 149418699 | 150093875 | 0.435 | NA | NA | NA | NA | NA |
| WCM131_Z1_1 | 7 | 150094218 | 158937375 | −3.00E−04 | NA | NA | NA | NA | NA |
| WCM131_Z1_1 | 8 | 190906 | 41845000 | 0.0232 | NA | NA | NA | NA | NA |
| WCM131_Z1_1 | 8 | 41906059 | 47757650 | 0.4114 | NA | NA | NA | NA | NA |
| WCM131_Z1_1 | 8 | 47759611 | 85097240 | 0.0129 | NA | NA | NA | NA | NA |
| WCM131_Z1_1 | 8 | 85441778 | 95895077 | 0.4035 | NA | NA | NA | NA | NA |
| WCM131_Z1_1 | 8 | 95896123 | 96087877 | −0.7941 | NA | NA | NA | NA | NA |
| WCM131_Z1_1 | 8 | 96166461 | 122629380 | 0.4323 | NA | NA | NA | NA | NA |
| WCM131_Z1_1 | 8 | 122641014 | 136561143 | 0.7553 | NA | NA | NA | NA | NA |
| WCM131_Z1_1 | 8 | 136569699 | 146157862 | 0.4791 | NA | NA | NA | NA | NA |
| WCM131_Z1_1 | 8 | 146171437 | 146279480 | −0.4805 | NA | NA | NA | NA | NA |
| WCM131_Z1_1 | 9 | 17079 | 35697908 | 0.0119 | NA | NA | NA | NA | NA |
| WCM131_Z1_1 | 9 | 35698051 | 35846538 | −0.5989 | NA | NA | NA | NA | NA |
| WCM131_Z1_1 | 9 | 35846950 | 79117591 | 0.0431 | NA | NA | NA | NA | NA |
| WCM131_Z1_1 | 9 | 79117919 | 80043826 | 0.3592 | NA | NA | NA | NA | NA |
| WCM131_Z1_1 | 9 | 80046238 | 119448979 | 0.0234 | NA | NA | NA | NA | NA |
| WCM131_Z1_1 | 9 | 119460046 | 119976803 | −1.1553 | NA | NA | NA | NA | NA |
| WCM131_Z1_1 | 9 | 119977007 | 123875986 | 0.0084 | NA | NA | NA | NA | NA |
| WCM131_Z1_1 | 9 | 123877378 | 126429322 | 0.4983 | NA | NA | NA | NA | NA |
| WCM131_Z1_1 | 9 | 126433572 | 139804431 | 0.0206 | NA | NA | NA | NA | NA |
| WCM131_Z1_1 | 9 | 139811005 | 140002847 | 0.4764 | NA | NA | NA | NA | NA |
| WCM131_Z1_1 | 9 | 140003117 | 141111414 | 0.0189 | NA | NA | NA | NA | NA |
| WCM131_Z1_1 | X | 2707709 | 154509348 | −0.0531 | NA | NA | NA | NA | NA |
| WCM131_Z2_1 | 1 | 69569 | 26857275 | −0.2072 | NA | NA | NA | NA | NA |
| WCM131_Z2_1 | 1 | 26863426 | 145598293 | −0.0047 | NA | NA | NA | NA | NA |
| WCM131_Z2_1 | 1 | 145598587 | 145704159 | 0.2086 | NA | NA | NA | NA | NA |
| WCM131_Z2_1 | 1 | 145704302 | 155282165 | 0.3809 | NA | NA | NA | NA | NA |
| WCM131_Z2_1 | 1 | 155287964 | 207236767 | 0.1913 | NA | NA | NA | NA | NA |
| WCM131_Z2_1 | 1 | 207238419 | 249212341 | −0.0155 | NA | NA | NA | NA | NA |
| WCM131_Z2_1 | 10 | 226016 | 3823864 | −0.0075 | NA | NA | NA | NA | NA |
| WCM131_Z2_1 | 10 | 3824107 | 14478576 | 0.234 | NA | NA | NA | NA | NA |
| WCM131_Z2_1 | 10 | 14563215 | 135440086 | 0.0048 | NA | NA | NA | NA | NA |
| WCM131_Z2_1 | 11 | 193100 | 1010694 | −0.0508 | NA | NA | NA | NA | NA |
| WCM131_Z2_1 | 11 | 1011194 | 1262175 | 0.1837 | NA | NA | NA | NA | NA |
| WCM131_Z2_1 | 11 | 1262541 | 27395582 | 0.0072 | NA | NA | NA | NA | NA |
| WCM131_Z2_1 | 11 | 27398717 | 57069403 | −0.1861 | NA | NA | NA | NA | NA |
| WCM131_Z2_1 | 11 | 57069695 | 82708344 | 0.0065 | NA | NA | NA | NA | NA |
| WCM131_Z2_1 | 11 | 82752580 | 134856542 | −0.257 | NA | NA | NA | NA | NA |
| WCM131_Z2_1 | 12 | 148673 | 27235073 | 0.1898 | NA | NA | NA | NA | NA |
| WCM131_Z2_1 | 12 | 27235334 | 48963795 | −0.0142 | NA | NA | NA | NA | NA |
| WCM131_Z2_1 | 12 | 49047845 | 95044189 | 0.2097 | NA | NA | NA | NA | NA |
| WCM131_Z2_1 | 12 | 95228244 | 133810892 | −0.0037 | NA | NA | NA | NA | NA |
| WCM131_Z2_1 | 13 | 19600515 | 39616254 | −0.0185 | NA | NA | NA | NA | NA |
| WCM131_Z2_1 | 13 | 39618262 | 75936731 | −0.2114 | NA | NA | NA | NA | NA |
| WCM131_Z2_1 | 13 | 76055514 | 115089411 | −0.0235 | NA | NA | NA | NA | NA |
| WCM131_Z2_1 | 14 | 19377701 | 107283183 | 0.006 | NA | NA | NA | NA | NA |
| WCM131_Z2_1 | 15 | 20169956 | 102506284 | 0.006 | NA | NA | NA | NA | NA |
| WCM131_Z2_1 | 16 | 67604 | 4621572 | −0.1874 | NA | NA | NA | NA | NA |
| WCM131_Z2_1 | 16 | 4624731 | 78466437 | −0.0088 | NA | NA | NA | NA | NA |
| WCM131_Z2_1 | 16 | 78466635 | 90161578 | −0.2014 | NA | NA | NA | NA | NA |
| WCM131_Z2_1 | 17 | 131595 | 1961350 | −0.154 | NA | NA | NA | NA | NA |
| WCM131_Z2_1 | 17 | 1961701 | 74681232 | 0.0114 | NA | NA | NA | NA | NA |
| WCM131_Z2_1 | 17 | 74684401 | 81083455 | −0.2051 | NA | NA | NA | NA | NA |
| WCM131_Z2_1 | 18 | 48992 | 78005210 | −0.0231 | NA | NA | NA | NA | NA |
| WCM131_Z2_1 | 19 | 281403 | 51631695 | 6.00E−04 | NA | NA | NA | NA | NA |
| WCM131_Z2_1 | 19 | 51633333 | 59083929 | 0.4953 | NA | NA | NA | NA | NA |
| WCM131_Z2_1 | 2 | 41614 | 31181320 | 0.2062 | NA | NA | NA | NA | NA |
| WCM131_Z2_1 | 2 | 31189155 | 241930672 | −0.0124 | NA | NA | NA | NA | NA |
| WCM131_Z2_1 | 2 | 241932484 | 242946604 | 0.0988 | NA | NA | NA | NA | NA |
| WCM131_Z2_1 | 20 | 68363 | 9360789 | −0.1845 | NA | NA | NA | NA | NA |
| WCM131_Z2_1 | 20 | 9364917 | 62904861 | 0.0167 | NA | NA | NA | NA | NA |
| WCM131_Z2_1 | 21 | 9826235 | 48084253 | −0.0198 | NA | NA | NA | NA | NA |
| WCM131_Z2_1 | 22 | 17060362 | 51215152 | 0.0081 | NA | NA | NA | NA | NA |
| WCM131_Z2_1 | 3 | 239431 | 43744017 | −0.0138 | NA | NA | NA | NA | NA |
| WCM131_Z2_1 | 3 | 43753334 | 59032925 | −0.2054 | NA | NA | NA | NA | NA |
| WCM131_Z2_1 | 3 | 59035342 | 197894719 | −0.0152 | NA | NA | NA | NA | NA |
| WCM131_Z2_1 | 4 | 59382 | 140325614 | −0.0248 | NA | NA | NA | NA | NA |
| WCM131_Z2_1 | 4 | 140339071 | 189061036 | 0.2284 | NA | NA | NA | NA | NA |
| WCM131_Z2_1 | 4 | 189061749 | 190426502 | −0.0349 | NA | NA | NA | NA | NA |
| WCM131_Z2_1 | 5 | 140636 | 1880865 | 0.2959 | NA | NA | NA | NA | NA |
| WCM131_Z2_1 | 5 | 2747681 | 157078475 | −0.0196 | NA | NA | NA | NA | NA |
| WCM131_Z2_1 | 5 | 157078670 | 180687814 | 0.1428 | NA | NA | NA | NA | NA |
| WCM131_Z2_1 | 6 | 203561 | 137113046 | −0.0208 | NA | NA | NA | NA | NA |
| WCM131_Z2_1 | 6 | 137113256 | 170893407 | 0.1542 | NA | NA | NA | NA | NA |
| WCM131_Z2_1 | 7 | 193205 | 158937416 | −0.0151 | NA | NA | NA | NA | NA |
| WCM131_Z2_1 | 8 | 190897 | 100836135 | −0.0117 | NA | NA | NA | NA | NA |
| WCM131_Z2_1 | 8 | 100844699 | 100974956 | 0.313 | NA | NA | NA | NA | NA |
| WCM131_Z2_1 | 8 | 100975194 | 123965175 | 0.1837 | NA | NA | NA | NA | NA |
| WCM131_Z2_1 | 8 | 123965501 | 146157545 | 0.3664 | NA | NA | NA | NA | NA |
| WCM131_Z2_1 | 8 | 146157781 | 146279508 | −0.3054 | NA | NA | NA | NA | NA |
| WCM131_Z2_1 | 9 | 17079 | 141111414 | −0.0021 | NA | NA | NA | NA | NA |
| WCM131_Z2_1 | X | 2707734 | 154509348 | −0.0966 | NA | NA | NA | NA | NA |
| WCM1388 | 1 | 69592 | 58522014 | 0.1518 | −0.0609 | 0.9587 | 0.9587 | 1 | 1 |
| WCM1388 | 1 | 58939502 | 119427436 | −0.6369 | −0.9213 | 1.0489 | 0.0072 | 1 | 0 |
| WCM1388 | 1 | 119427676 | 154474377 | 0.323 | 0.121 | 1.099 | 1.076 | 1 | 1 |
| WCM1388 | 1 | 154479415 | 155649291 | 0.8946 | 0.72 | 2.3572 | 0.9371 | 2 | 1 |
| WCM1388 | 1 | 155658085 | 249212354 | 0.144 | −0.0692 | 0.9531 | 0.9531 | 1 | 1 |
| WCM1388 | 10 | 225982 | 81113556 | 0.1558 | −0.0567 | 0.9615 | 0.9615 | 1 | 1 |
| WCM1388 | 10 | 81146112 | 96109022 | −0.7514 | −1.0502 | 0.9739 | −0.0081 | 1 | 0 |
| WCM1388 | 10 | 96110038 | 117825099 | −0.1656 | −0.4021 | 1.0152 | 0.4983 | 1 | 0 |
| WCM1388 | 10 | 117849361 | 118117368 | −0.8099 | −1.1167 | NA | NA | NA | NA |
| WCM1388 | 10 | 118137967 | 135378918 | 0.2354 | 0.0281 | 1.024 | 1.0153 | 1 | 1 |
| WCM1388 | 11 | 193096 | 134257753 | 0.0572 | −0.1621 | 0.8937 | 0.8937 | 1 | 1 |
| WCM1388 | 12 | 148642 | 133810905 | 0.17 | −0.0415 | 0.9716 | 0.9716 | 1 | 1 |
| WCM1388 | 13 | 19600326 | 33241911 | 0.2022 | −0.0072 | 1.0003 | 0.9897 | 1 | 1 |
| WCM1388 | 13 | 33247408 | 76445044 | −0.6502 | −0.9362 | 1.0487 | −0.0034 | 1 | 0 |
| WCM1388 | 13 | 76447830 | 113153315 | 0.123 | −0.0916 | 0.9435 | 0.9334 | 1 | 1 |
| WCM1388 | 13 | 113153523 | 115089411 | −0.5887 | −0.8674 | 1.0928 | 0.0035 | 1 | 0 |
| WCM1388 | 14 | 19110187 | 107283026 | 0.4248 | 0.2285 | 1.399 | 0.9443 | 1 | 1 |
| WCM1388 | 15 | 20083905 | 102506240 | −0.6994 | −0.9915 | 1.013 | −0.0072 | 1 | 0 |
| WCM1388 | 16 | 67166 | 73177632 | 0.1405 | −0.0729 | 0.9507 | 0.9507 | 1 | 1 |
| WCM1388 | 16 | 74330836 | 90110890 | −0.6543 | −0.9408 | 1.0414 | 5.00E−04 | 1 | 0 |
| WCM1388 | 16 | 90124832 | 90161568 | 0.3463 | 0.1456 | NA | NA | NA | NA |
| WCM1388 | 17 | 131595 | 7960947 | −0.7137 | −1.0077 | 1.0018 | −0.0072 | 1 | 0 |
| WCM1388 | 17 | 7976212 | 81052262 | 0.1774 | −0.0337 | 0.9769 | 0.9769 | 1 | 1 |
| WCM1388 | 18 | 47653 | 13826391 | 0.2 | −0.0096 | 0.9934 | 0.9934 | 1 | 1 |
| WCM1388 | 18 | 13826508 | 23866281 | 0.6275 | 0.4415 | 1.6893 | 1.0267 | 2 | 1 |
| WCM1388 | 18 | 23866482 | 29672894 | 0.1486 | −0.0643 | 0.9891 | 0.9238 | 1 | 1 |
| WCM1388 | 18 | 29691756 | 78005210 | −0.7573 | −1.057 | 0.969 | −0.0077 | 1 | 0 |
| WCM1388 | 19 | 281506 | 1555325 | −0.0196 | −0.2445 | 0.8605 | 0.8278 | 1 | 1 |
| WCM1388 | 19 | 1555725 | 2643436 | −0.6196 | −0.9019 | 1.0737 | −0.0033 | 1 | 0 |
| WCM1388 | 19 | 2717256 | 11436268 | 0.287 | 0.0829 | 1.0591 | 1.0591 | 1 | 1 |
| WCM1388 | 19 | 11446278 | 14561908 | −0.4419 | −0.7044 | 1.2289 | −0.0015 | 1 | 0 |
| WCM1388 | 19 | 14562848 | 43016548 | 0.2604 | 0.0547 | 1.0386 | 1.0386 | 1 | 1 |
| WCM1388 | 19 | 43023080 | 43858093 | 2.242 | 2.101 | 4.3554 | 4.2248 | 4 | 4 |
| WCM1388 | 19 | 43858442 | 52873183 | 0.1983 | −0.0114 | 0.9922 | 0.9922 | 1 | 1 |
| WCM1388 | 19 | 52876385 | 54265722 | 1.3852 | 1.2266 | 2.3402 | 2.3402 | 2 | 2 |
| WCM1388 | 19 | 54290902 | 59083985 | 0.3315 | 0.13 | 1.107 | 1.0815 | 1 | 1 |
| WCM1388 | 2 | 41509 | 33952621 | −0.1689 | −0.4057 | 0.9784 | 0.5314 | 1 | 1 |
| WCM1388 | 2 | 33952867 | 43514248 | −0.6197 | −0.902 | 1.0658 | 0.0045 | 1 | 0 |
| WCM1388 | 2 | 43518858 | 242946616 | −0.1513 | −0.3867 | 0.9994 | 0.5304 | 1 | 1 |
| WCM1388 | 20 | 68375 | 62905001 | 0.1172 | −0.0979 | 0.9344 | 0.9344 | 1 | 1 |
| WCM1388 | 21 | 9907270 | 48084163 | 0.1903 | −0.0199 | 0.9916 | 0.9811 | 1 | 1 |
| WCM1388 | 22 | 17072119 | 51219129 | −0.7158 | −1.01 | 0.9953 | −0.0022 | 1 | 0 |
| WCM1388 | 3 | 239405 | 16645969 | 0.1903 | −0.0199 | 0.9863 | 0.9863 | 1 | 1 |
| WCM1388 | 3 | 16679531 | 29910445 | −0.7221 | −1.0171 | 1.0107 | −0.0225 | 1 | 0 |
| WCM1388 | 3 | 29922333 | 35758749 | 0.2747 | 0.0698 | 1.1376 | 0.9616 | 1 | 1 |
| WCM1388 | 3 | 35763202 | 89521725 | −0.7853 | −1.0886 | 0.9508 | −0.0104 | 1 | 0 |
| WCM1388 | 3 | 89528644 | 197894744 | 0.6809 | 0.4974 | 1.8543 | 0.969 | 2 | 1 |
| WCM1388 | 4 | 53349 | 77039174 | −0.1072 | −0.3389 | 1.0196 | 0.5616 | 1 | 1 |
| WCM1388 | 4 | 77039412 | 152096297 | −0.6747 | −0.9637 | 0.5168 | 0.5087 | 1 | 1 |
| WCM1388 | 4 | 152096526 | 190396290 | −0.1837 | −0.4218 | 0.9596 | 0.5334 | 1 | 1 |
| WCM1388 | 5 | 140598 | 70942059 | 0.1877 | −0.0227 | 0.9844 | 0.9844 | 1 | 1 |
| WCM1388 | 5 | 70944988 | 74877140 | −0.6709 | −0.9594 | 1.0311 | −0.0026 | 1 | 0 |
| WCM1388 | 5 | 74879106 | 180687800 | 0.1401 | −0.0734 | 0.9504 | 0.9504 | 1 | 1 |
| WCM1388 | 6 | 203588 | 89673141 | 0.1708 | −0.0406 | 0.9722 | 0.9722 | 1 | 1 |
| WCM1388 | 6 | 89675638 | 131894392 | −0.7239 | −1.0191 | 0.9988 | −0.012 | 1 | 0 |
| WCM1388 | 6 | 131895418 | 132859437 | −0.0348 | −0.2608 | NA | NA | NA | NA |
| WCM1388 | 6 | 132859749 | 143094724 | 0.146 | −0.0671 | 0.978 | 0.9311 | 1 | 1 |
| WCM1388 | 6 | 143095045 | 170893411 | −0.2292 | −0.4713 | 0.9537 | 0.4889 | 1 | 0 |
| WCM1388 | 7 | 193205 | 5360661 | 0.2682 | 0.0629 | 1.0667 | 1.0224 | 1 | 1 |
| WCM1388 | 7 | 5363981 | 56879527 | −0.7139 | −1.0079 | 0.9961 | −0.0015 | 1 | 0 |
| WCM1388 | 7 | 57194311 | 158937375 | −0.1787 | −0.4164 | 0.9648 | 0.5338 | 1 | 1 |
| WCM1388 | 8 | 190907 | 48686936 | −0.6999 | −0.992 | 1.0116 | −0.006 | 1 | 0 |
| WCM1388 | 8 | 48689448 | 87242489 | 0.7946 | 0.616 | 2.0978 | 0.9675 | 2 | 1 |
| WCM1388 | 8 | 87386324 | 110590046 | 1.254 | 1.0917 | 3.275 | 0.9876 | 3 | 1 |
| WCM1388 | 8 | 110592226 | 114290824 | −0.5751 | −0.8522 | NA | NA | NA | NA |
| WCM1388 | 8 | 114326961 | 131848465 | 1.2555 | 1.0933 | 3.2257 | 1.0415 | 3 | 1 |
| WCM1388 | 8 | 131848728 | 143395808 | 0.7506 | 0.5701 | 1.9538 | 1.0154 | 2 | 1 |
| WCM1388 | 8 | 143396408 | 146279441 | −0.8508 | −1.1633 | 0.9089 | −0.0159 | 1 | 0 |
| WCM1388 | 9 | 116887 | 141071710 | 0.1721 | −0.0393 | 0.9731 | 0.9731 | 1 | 1 |
| WCM1388 | X | 2707715 | 154509348 | 0.1217 | −0.0931 | NA | NA | NA | NA |
| WCM1388_Z3_1 | 1 | 69590 | 57756687 | 0.168 | 0.0258 | 1.018 | 1.018 | 1 | 1 |
| WCM1388_Z3_1 | 1 | 58521986 | 119427454 | −0.7627 | −1.005 | 1.0024 | −0.0059 | 1 | 0 |
| WCM1388_Z3_1 | 1 | 119427796 | 154442830 | 0.1511 | 0.0077 | 1.0518 | 0.9589 | 1 | 1 |
| WCM1388_Z3_1 | 1 | 154456643 | 155631029 | 1.0715 | 0.9781 | 2.9209 | 1.0189 | 3 | 1 |
| WCM1388_Z3_1 | 1 | 155638491 | 249212432 | 0.1256 | −0.0198 | 0.9938 | 0.9789 | 1 | 1 |
| WCM1388_Z3_1 | 10 | 225960 | 81111240 | 0.1296 | −0.0154 | 0.9894 | 0.9894 | 1 | 1 |
| WCM1388_Z3_1 | 10 | 81112114 | 96356617 | −0.7764 | −1.0208 | 1.0013 | −0.0156 | 1 | 0 |
| WCM1388_Z3_1 | 10 | 96443723 | 120446120 | −0.2406 | −0.4181 | 0.9972 | 0.4997 | 1 | 0 |
| WCM1388_Z3_1 | 10 | 120450859 | 135440080 | 0.1362 | −0.0084 | 1.007 | 0.9813 | 1 | 1 |
| WCM1388_Z3_1 | 11 | 193097 | 134856512 | 0.1369 | −0.0076 | 1.0011 | 0.9883 | 1 | 1 |
| WCM1388_Z3_1 | 12 | 148639 | 133810892 | 0.1418 | −0.0023 | 1.0048 | 0.9919 | 1 | 1 |
| WCM1388_Z3_1 | 13 | 19600364 | 33241969 | 0.13 | −0.0151 | 1.0237 | 0.9555 | 1 | 1 |
| WCM1388_Z3_1 | 13 | 33247409 | 76457200 | −0.764 | −1.0065 | 1.0307 | −0.0352 | 1 | 0 |
| WCM1388_Z3_1 | 13 | 77459417 | 80122525 | 0.1103 | −0.0362 | NA | NA | NA | NA |
| WCM1388_Z3_1 | 13 | 80125207 | 92345556 | −0.8499 | −1.1058 | NA | NA | NA | NA |
| WCM1388_Z3_1 | 13 | 92345724 | 113088860 | 0.1267 | −0.0186 | 1.001 | 0.9734 | 1 | 1 |
| WCM1388_Z3_1 | 13 | 113144002 | 115089411 | −0.7386 | −0.9773 | 0.9984 | 0.0175 | 1 | 0 |
| WCM1388_Z3_1 | 14 | 19377716 | 60591863 | 0.4234 | 0.2984 | 1.4817 | 0.9779 | 1 | 1 |
| WCM1388_Z3_1 | 14 | 60592390 | 62462947 | −0.2088 | −0.3832 | NA | NA | NA | NA |
| WCM1388_Z3_1 | 14 | 62463214 | 107283220 | 0.4359 | 0.3116 | 1.4899 | 0.9923 | 1 | 1 |
| WCM1388_Z3_1 | 15 | 20170159 | 22319166 | −1.5028 | −1.8991 | NA | NA | NA | NA |
| WCM1388_Z3_1 | 15 | 22319565 | 23685583 | −0.7359 | −0.9742 | 0.8024 | 0.2157 | 1 | 0 |
| WCM1388_Z3_1 | 15 | 23686112 | 102506282 | −0.7473 | −0.9872 | 1.0135 | −0.0045 | 1 | 0 |
| WCM1388_Z3_1 | 16 | 67604 | 73521333 | 0.1595 | 0.0167 | 1.0225 | 1.0007 | 1 | 1 |
| WCM1388_Z3_1 | 16 | 74321131 | 90233603 | −0.7193 | −0.9551 | 1.0277 | 0.0039 | 1 | 0 |
| WCM1388_Z3_1 | 17 | 131595 | 7960881 | −0.741 | −0.98 | 1.0188 | −0.0048 | 1 | 0 |
| WCM1388_Z3_1 | 17 | 7976137 | 81083567 | 0.1612 | 0.0185 | 1.0129 | 1.0129 | 1 | 1 |
| WCM1388_Z3_1 | 18 | 48339 | 21033462 | 0.1395 | −0.0048 | 1.0042 | 0.9892 | 1 | 1 |
| WCM1388_Z3_1 | 18 | 21042940 | 23901053 | 0.6808 | 0.5702 | 2.0291 | 0.9403 | 2 | 1 |
| WCM1388_Z3_1 | 18 | 23907073 | 29691764 | 0.0702 | −0.0795 | NA | NA | NA | NA |
| WCM1388_Z3_1 | 18 | 29770050 | 78005206 | −0.7625 | −1.0048 | 1.0077 | −0.011 | 1 | 0 |
| WCM1388_Z3_1 | 19 | 281534 | 1556921 | 0.1709 | 0.0289 | 1.064 | 0.9764 | 1 | 1 |
| WCM1388_Z3_1 | 19 | 1567500 | 2643479 | −0.6975 | −0.9302 | 1.057 | −0.0075 | 1 | 0 |
| WCM1388_Z3_1 | 19 | 2717256 | 11422816 | 0.1587 | 0.0159 | 1.011 | 1.011 | 1 | 1 |
| WCM1388_Z3_1 | 19 | 11436119 | 14574916 | −0.7198 | −0.9557 | 1.0326 | −0.0014 | 1 | 0 |
| WCM1388_Z3_1 | 19 | 14578566 | 59083970 | 0.1306 | −0.0144 | 0.9954 | 0.9848 | 1 | 1 |
| WCM1388_Z3_1 | 2 | 41609 | 33952621 | −0.2299 | −0.4064 | 0.9918 | 0.5172 | 1 | 1 |
| WCM1388_Z3_1 | 2 | 36605503 | 43452793 | −0.714 | −0.949 | 1.0021 | 0.0339 | 1 | 0 |
| WCM1388_Z3_1 | 2 | 43453408 | 242946589 | −0.2508 | −0.4294 | 0.9904 | 0.4948 | 1 | 0 |
| WCM1388_Z3_1 | 20 | 68351 | 62904890 | 0.1478 | 0.0041 | 1.0071 | 0.9985 | 1 | 1 |
| WCM1388_Z3_1 | 21 | 9907283 | 48084282 | 0.119 | −0.0269 | 0.9899 | 0.973 | 1 | 1 |
| WCM1388_Z3_1 | 22 | 17063523 | 51215157 | −0.7289 | −0.9661 | 1.0221 | 0.0016 | 1 | 0 |
| WCM1388_Z3_1 | 3 | 239431 | 16640013 | 0.156 | 0.0129 | 1.0296 | 0.9884 | 1 | 1 |
| WCM1388_Z3_1 | 3 | 16645993 | 27763624 | −0.7709 | −1.0144 | NA | NA | NA | NA |
| WCM1388_Z3_1 | 3 | 28283353 | 29804441 | 0.1008 | −0.0465 | NA | NA | NA | NA |
| WCM1388_Z3_1 | 3 | 29910449 | 32767034 | 0.7263 | 0.618 | NA | NA | NA | NA |
| WCM1388_Z3_1 | 3 | 32769191 | 36485003 | 0.0819 | −0.0669 | NA | NA | NA | NA |
| WCM1388_Z3_1 | 3 | 36524545 | 79638984 | −0.7359 | −0.9742 | 1.0226 | −0.0046 | 1 | 0 |
| WCM1388_Z3_1 | 3 | 81539593 | 89521693 | 0.1601 | 0.0173 | NA | NA | NA | NA |
| WCM1388_Z3_1 | 3 | 89528635 | 197894726 | 0.6739 | 0.5629 | 1.9679 | 0.9867 | 2 | 1 |
| WCM1388_Z3_1 | 4 | 59339 | 74606389 | −0.2261 | −0.4022 | 0.9905 | 0.5229 | 1 | 1 |
| WCM1388_Z3_1 | 4 | 74607384 | 152096321 | −0.7608 | −1.0028 | 0.557 | 0.441 | 1 | 0 |
| WCM1388_Z3_1 | 4 | 152198354 | 190426471 | −0.2556 | −0.4347 | 0.9827 | 0.4969 | 1 | 0 |
| WCM1388_Z3_1 | 5 | 140608 | 70900240 | 0.1163 | −0.0299 | 0.9932 | 0.9658 | 1 | 1 |
| WCM1388_Z3_1 | 5 | 70922536 | 74801833 | −0.7221 | −0.9583 | 1.035 | −0.0057 | 1 | 0 |
| WCM1388_Z3_1 | 5 | 74806969 | 180687814 | 0.0936 | −0.0543 | 0.9724 | 0.9537 | 1 | 1 |
| WCM1388_Z3_1 | 6 | 203516 | 89601562 | 0.129 | −0.0162 | 0.9952 | 0.9825 | 1 | 1 |
| WCM1388_Z3_1 | 6 | 89614462 | 131603572 | −0.7736 | −1.0175 | 0.9978 | −0.0098 | 1 | 0 |
| WCM1388_Z3_1 | 6 | 131894425 | 143095570 | 0.1412 | −0.003 | 1.0419 | 0.9539 | 1 | 1 |
| WCM1388_Z3_1 | 6 | 143095824 | 170893454 | −0.2547 | −0.4336 | 0.9647 | 0.5161 | 1 | 1 |
| WCM1388_Z3_1 | 7 | 193222 | 803563 | 0.0463 | −0.1053 | NA | NA | NA | NA |
| WCM1388_Z3_1 | 7 | 808827 | 1579908 | 0.4451 | 0.3214 | 1.3423 | 1.1568 | 1 | 1 |
| WCM1388_Z3_1 | 7 | 1586662 | 5028762 | 0.1728 | 0.0309 | 1.048 | 0.9953 | 1 | 1 |
| WCM1388_Z3_1 | 7 | 5036270 | 62758670 | −0.7563 | −0.9976 | 1.0009 | 8.00E−04 | 1 | 0 |
| WCM1388_Z3_1 | 7 | 63529270 | 158937375 | −0.3178 | −0.5034 | 0.9428 | 0.4681 | 1 | 0 |
| WCM1388_Z3_1 | 8 | 190900 | 47753945 | −0.7506 | −0.991 | 1.0109 | −0.0047 | 1 | 0 |
| WCM1388_Z3_1 | 8 | 47755043 | 65291808 | 0.5544 | 0.4371 | 1.8883 | 0.8194 | 2 | 1 |
| WCM1388_Z3_1 | 8 | 65489055 | 82571682 | 1.0211 | 0.9258 | 2.7706 | 1.0287 | 3 | 1 |
| WCM1388_Z3_1 | 8 | 82572782 | 92364100 | 0.7491 | 0.6419 | 2.1819 | 0.9388 | 2 | 1 |
| WCM1388_Z3_1 | 8 | 92365182 | 105839705 | 1.3632 | 1.2799 | 3.9528 | 0.9036 | 4 | 1 |
| WCM1388_Z3_1 | 8 | 105907645 | 110703578 | 1.0727 | 0.9793 | 2.9888 | 0.9543 | 3 | 1 |
| WCM1388_Z3_1 | 8 | 110980424 | 114111040 | −0.7258 | −0.9626 | NA | NA | NA | NA |
| WCM1388_Z3_1 | 8 | 114186003 | 120431585 | 0.9628 | 0.8651 | NA | NA | NA | NA |
| WCM1388_Z3_1 | 8 | 120435302 | 131796024 | 1.3253 | 1.2408 | 3.741 | 0.9855 | 4 | 1 |
| WCM1388_Z3_1 | 8 | 131797655 | 132002781 | 0.8639 | 0.7619 | NA | NA | NA | NA |
| WCM1388_Z3_1 | 8 | 132051784 | 141856708 | 0.6638 | 0.5523 | 1.9104 | 1.0224 | 2 | 1 |
| WCM1388_Z3_1 | 8 | 141889725 | 143381993 | 1.0771 | 0.9839 | NA | NA | NA | NA |
| WCM1388_Z3_1 | 8 | 143395750 | 146279441 | −0.7273 | −0.9643 | 1.0257 | −6.00E−04 | 1 | 0 |
| WCM1388_Z3_1 | 9 | 17049 | 2047423 | 0.1319 | −0.013 | NA | NA | NA | NA |
| WCM1388_Z3_1 | 9 | 2054640 | 3248083 | −0.7663 | −1.0091 | NA | NA | NA | NA |
| WCM1388_Z3_1 | 9 | 3257165 | 141111414 | 0.1358 | −0.0088 | 0.9982 | 0.9896 | 1 | 1 |
| WCM1388_Z3_1 | X | 2707708 | 154509348 | 0.0714 | −0.0782 | NA | NA | NA | NA |
| WCM139 | 1 | 17479 | 249213973 | 0.0271 | 0.1771 | 1.1306 | 1.1306 | 1 | 1 |
| WCM139 | 10 | 225980 | 135477892 | 0.0153 | 0.0179 | 1.0125 | 1.0125 | 1 | 1 |
| WCM139 | 11 | 193152 | 62327315 | 0.0269 | 0.1748 | 1.1288 | 1.1288 | 1 | 1 |
| WCM139 | 11 | 62327496 | 66453823 | −0.0869 | −4.8337 | 0.115 | −0.0449 | 0 | 0 |
| WCM139 | 11 | 66453849 | 134605906 | 0.0137 | −0.0046 | 0.9968 | 0.9968 | 1 | 1 |
| WCM139 | 12 | 148560 | 133810905 | −0.0065 | −0.3247 | 0.7985 | 0.7985 | 1 | 1 |
| WCM139 | 13 | 19042004 | 115089411 | 0.0014 | −0.1917 | 0.8756 | 0.8756 | 1 | 1 |
| WCM139 | 14 | 19110343 | 107283263 | 0.0049 | −0.1358 | 0.9102 | 0.9102 | 1 | 1 |
| WCM139 | 15 | 20083857 | 102514263 | 0.0231 | 0.125 | 1.0905 | 1.0905 | 1 | 1 |
| WCM139 | 16 | 64396 | 90260461 | 0.0035 | −0.1575 | 0.8966 | 0.8966 | 1 | 1 |
| WCM139 | 17 | 131595 | 81052551 | 0.0108 | −0.0471 | 0.9679 | 0.9679 | 1 | 1 |
| WCM139 | 18 | 47611 | 78005194 | 0.0174 | 0.0479 | 1.0338 | 1.0338 | 1 | 1 |
| WCM139 | 19 | 105080 | 59093645 | −0.0243 | −0.6768 | 0.6255 | 0.6255 | 1 | 1 |
| WCM139 | 2 | 41647 | 243037045 | 0.0022 | −0.179 | 0.8833 | 0.8833 | 1 | 1 |
| WCM139 | 20 | 68373 | 62934617 | 0.0427 | 0.3645 | 1.2874 | 1.2874 | 1 | 1 |
| WCM139 | 21 | 9590305 | 48084167 | 0.0106 | −0.049 | 0.9666 | 0.9666 | 1 | 1 |
| WCM139 | 22 | 16159016 | 51223730 | 0.05 | 0.445 | 1.3613 | 1.3613 | 1 | 1 |
| WCM139 | 3 | 239377 | 197896678 | 0.0155 | 0.0212 | 1.0148 | 1.0148 | 1 | 1 |
| WCM139 | 4 | 53333 | 190878508 | 0.0113 | −0.0384 | 0.9737 | 0.9737 | 1 | 1 |
| WCM139 | 5 | 90680 | 180687800 | −0.0056 | −0.3089 | 0.8072 | 0.8072 | 1 | 1 |
| WCM139 | 6 | 203466 | 170892848 | −0.0033 | −0.2687 | 0.8301 | 0.8301 | 1 | 1 |
| WCM139 | 7 | 193222 | 158937074 | 0.006 | −0.118 | 0.9215 | 0.9215 | 1 | 1 |
| WCM139 | 8 | 163527 | 146279431 | 0.0237 | 0.1325 | 1.0962 | 1.0962 | 1 | 1 |
| WCM139 | 9 | 14856 | 141107539 | 0.0285 | 0.1945 | 1.1443 | 1.1443 | 1 | 1 |
| WCM139 | X | 2700082 | 154493585 | −0.0842 | −4.0575 | NA | NA | NA | NA |
| WCM139_Z3_1 | 1 | 721450 | 29696240 | −0.2217 | 0.0065 | 1.0194 | 0.9896 | 1 | 1 |
| WCM139_Z3_1 | 1 | 31186427 | 33502336 | 0.253 | 0.5727 | NA | NA | NA | NA |
| WCM139_Z3_1 | 1 | 33547875 | 43042703 | −0.2242 | 0.0035 | 1.0148 | 0.99 | 1 | 1 |
| WCM139_Z3_1 | 1 | 43047057 | 46212067 | 0.7792 | 1.1668 | 2.337 | 2.1535 | 2 | 2 |
| WCM139_Z3_1 | 1 | 46290201 | 51121156 | −0.1234 | 0.1269 | 1.2073 | 0.9765 | 1 | 1 |
| WCM139_Z3_1 | 1 | 51171529 | 54871659 | 0.697 | 1.0757 | 3.3211 | 0.8944 | 3 | 1 |
| WCM139_Z3_1 | 1 | 55014015 | 76349378 | −0.1932 | 0.0416 | 1.0469 | 1.0116 | 1 | 1 |
| WCM139_Z3_1 | 1 | 76356455 | 84348786 | 0.4004 | 0.7421 | NA | NA | NA | NA |
| WCM139_Z3_1 | 1 | 84352964 | 148022941 | −0.2378 | −0.0133 | 1.0202 | 0.9614 | 1 | 1 |
| WCM139_Z3_1 | 1 | 149871721 | 150082689 | 0.1435 | 0.4451 | NA | NA | NA | NA |
| WCM139_Z3_1 | 1 | 150117036 | 150830834 | 0.9386 | 1.3419 | NA | NA | NA | NA |
| WCM139_Z3_1 | 1 | 150899274 | 151823590 | 0.2637 | 0.5851 | NA | NA | NA | NA |
| WCM139_Z3_1 | 1 | 151824852 | 153911501 | −0.2838 | −0.0705 | 0.9986 | 0.906 | 1 | 1 |
| WCM139_Z3_1 | 1 | 153912245 | 156316586 | 0.6082 | 0.9767 | NA | NA | NA | NA |
| WCM139_Z3_1 | 1 | 156337578 | 156639578 | 0.2643 | 0.5858 | NA | NA | NA | NA |
| WCM139_Z3_1 | 1 | 156639753 | 156670774 | 0.3366 | 0.6691 | NA | NA | NA | NA |
| WCM139_Z3_1 | 1 | 156675190 | 171958254 | −0.2252 | 0.0022 | 1.0337 | 0.9694 | 1 | 1 |
| WCM139_Z3_1 | 1 | 172007529 | 203465342 | 0.2475 | 0.5663 | 1.9679 | 0.9936 | 2 | 1 |
| WCM139_Z3_1 | 1 | 203466231 | 206325358 | 0.649 | 1.0223 | 2.0606 | 2.0017 | 2 | 2 |
| WCM139_Z3_1 | 1 | 206328803 | 213445925 | 0.2806 | 0.6047 | 2.0086 | 1.0327 | 2 | 1 |
| WCM139_Z3_1 | 1 | 214170023 | 215820956 | 0.9015 | 1.3013 | NA | NA | NA | NA |
| WCM139_Z3_1 | 1 | 215821999 | 233807102 | 0.2426 | 0.5607 | 1.9195 | 1.0305 | 2 | 1 |
| WCM139_Z3_1 | 1 | 233807277 | 236744544 | 0.9716 | 1.3779 | NA | NA | NA | NA |
| WCM139_Z3_1 | 1 | 236746079 | 249212557 | 0.2398 | 0.5574 | 1.946 | 0.9973 | 2 | 1 |
| WCM139_Z3_1 | 10 | 225988 | 3824017 | −0.2368 | −0.0121 | 1.0284 | 0.9549 | 1 | 1 |
| WCM139_Z3_1 | 10 | 3824319 | 6557064 | 0.6177 | 0.9874 | NA | NA | NA | NA |
| WCM139_Z3_1 | 10 | 7205750 | 13900896 | −0.2459 | −0.0234 | 1.0071 | 0.9608 | 1 | 1 |
| WCM139_Z3_1 | 10 | 14013711 | 17738786 | 0.4191 | 0.7633 | 2.0143 | 1.3806 | 2 | 1 |
| WCM139_Z3_1 | 10 | 17742207 | 71175693 | −0.208 | 0.0234 | 1.0351 | 0.9976 | 1 | 1 |
| WCM139_Z3_1 | 10 | 71176012 | 74322689 | 0.2551 | 0.5752 | 1.9469 | 1.0329 | 2 | 1 |
| WCM139_Z3_1 | 10 | 74326500 | 79396145 | −0.2363 | −0.0115 | NA | NA | NA | NA |
| WCM139_Z3_1 | 10 | 79397272 | 81051929 | 0.1903 | 0.4999 | NA | NA | NA | NA |
| WCM139_Z3_1 | 10 | 81052593 | 119044964 | −0.2216 | 0.0067 | 1.0183 | 0.991 | 1 | 1 |
| WCM139_Z3_1 | 10 | 119049804 | 123687875 | 0.2439 | 0.5622 | NA | NA | NA | NA |
| WCM139_Z3_1 | 10 | 123718917 | 126370945 | −0.1895 | 0.0462 | 1.0756 | 0.9895 | 1 | 1 |
| WCM139_Z3_1 | 10 | 126384749 | 134261520 | 0.0487 | 0.3334 | 1.6141 | 0.9057 | 2 | 1 |
| WCM139_Z3_1 | 10 | 134351675 | 135440090 | −0.2456 | −0.0229 | 1.0391 | 0.9294 | 1 | 1 |
| WCM139_Z3_1 | 11 | 193100 | 19258908 | −0.2223 | 0.0059 | 1.0239 | 0.9842 | 1 | 1 |
| WCM139_Z3_1 | 11 | 19259453 | 33308189 | 0.1289 | 0.4281 | 1.7641 | 0.9267 | 2 | 1 |
| WCM139_Z3_1 | 11 | 33308347 | 34938265 | −0.2122 | 0.0183 | 1.0602 | 0.9654 | 1 | 1 |
| WCM139_Z3_1 | 11 | 34963416 | 36680592 | 0.4028 | 0.7448 | NA | NA | NA | NA |
| WCM139_Z3_1 | 11 | 40136001 | 64627769 | −0.215 | 0.0148 | 1.0352 | 0.9854 | 1 | 1 |
| WCM139_Z3_1 | 11 | 64641974 | 65063051 | 0.5859 | 0.9517 | NA | NA | NA | NA |
| WCM139_Z3_1 | 11 | 65063487 | 65147603 | 0.8579 | 1.2535 | NA | NA | NA | NA |
| WCM139_Z3_1 | 11 | 65148047 | 65628185 | 1.1235 | 1.5428 | 3.8643 | 1.9628 | 4 | 2 |
| WCM139_Z3_1 | 11 | 65628553 | 66360473 | 0.6212 | 0.9913 | 1.9879 | 1.9879 | 2 | 2 |
| WCM139_Z3_1 | 11 | 66360760 | 66619263 | 0.2419 | 0.5598 | NA | NA | NA | NA |
| WCM139_Z3_1 | 11 | 66620052 | 67290084 | 0.5749 | 0.9394 | 2.8067 | 1.0288 | 3 | 1 |
| WCM139_Z3_1 | 11 | 67351627 | 70333182 | 0.2407 | 0.5585 | 2.0242 | 0.9213 | 2 | 1 |
| WCM139_Z3_1 | 11 | 70333611 | 85671739 | −0.1924 | 0.0426 | 1.1716 | 0.8883 | 1 | 1 |
| WCM139_Z3_1 | 11 | 85687682 | 87847223 | 0.5986 | 0.9661 | NA | NA | NA | NA |
| WCM139_Z3_1 | 11 | 87882970 | 100999754 | −0.2305 | −0.0043 | 1.0783 | 0.9158 | 1 | 1 |
| WCM139_Z3_1 | 11 | 101323826 | 103339409 | 0.3667 | 0.7036 | 1.8316 | 1.4255 | 2 | 1 |
| WCM139_Z3_1 | 11 | 103349844 | 117857338 | −0.2445 | −0.0216 | 1.0083 | 0.962 | 1 | 1 |
| WCM139_Z3_1 | 11 | 117857450 | 118851234 | 0.2633 | 0.5846 | 2.006 | 0.9932 | 2 | 1 |
| WCM139_Z3_1 | 11 | 118867772 | 128856536 | −0.2419 | −0.0184 | 1.0118 | 0.9629 | 1 | 1 |
| WCM139_Z3_1 | 11 | 128857968 | 134856597 | 0.1715 | 0.478 | 1.8419 | 0.9436 | 2 | 1 |
| WCM139_Z3_1 | 12 | 148674 | 6730347 | −0.2266 | 4.00E−04 | 1.0374 | 0.9633 | 1 | 1 |
| WCM139_Z3_1 | 12 | 6747529 | 10366463 | 0.2667 | 0.5886 | 2.0479 | 0.9597 | 2 | 1 |
| WCM139_Z3_1 | 12 | 10373232 | 40619418 | −0.2258 | 0.0015 | 1.0282 | 0.9739 | 1 | 1 |
| WCM139_Z3_1 | 12 | 40626183 | 43750297 | 0.2471 | 0.5658 | 1.969 | 0.9915 | 2 | 1 |
| WCM139_Z3_1 | 12 | 43763113 | 52345582 | −0.2269 | 1.00E−04 | 1.0124 | 0.9877 | 1 | 1 |
| WCM139_Z3_1 | 12 | 52369080 | 54625185 | 0.2807 | 0.6048 | 2.0333 | 1.0082 | 2 | 1 |
| WCM139_Z3_1 | 12 | 54635536 | 57146079 | −0.2391 | −0.0149 | 1.0607 | 0.9187 | 1 | 1 |
| WCM139_Z3_1 | 12 | 57167675 | 59284534 | 0.2581 | 0.5786 | 1.9772 | 1.0096 | 2 | 1 |
| WCM139_Z3_1 | 12 | 59307786 | 68052493 | −0.1336 | 0.1145 | 1.1432 | 1.022 | 1 | 1 |
| WCM139_Z3_1 | 12 | 68549153 | 71533533 | 0.2691 | 0.5914 | 2.0761 | 0.9373 | 2 | 1 |
| WCM139_Z3_1 | 12 | 71537944 | 79611374 | −0.2346 | −0.0094 | 1.0807 | 0.9063 | 1 | 1 |
| WCM139_Z3_1 | 12 | 79679648 | 83324244 | 0.5022 | 0.8576 | NA | NA | NA | NA |
| WCM139_Z3_1 | 12 | 83358857 | 88374127 | −0.2777 | −0.0629 | NA | NA | NA | NA |
| WCM139_Z3_1 | 12 | 88376908 | 91572214 | 0.2917 | 0.6175 | NA | NA | NA | NA |
| WCM139_Z3_1 | 12 | 92380427 | 122243074 | −0.2186 | 0.0104 | 1.032 | 0.9824 | 1 | 1 |
| WCM139_Z3_1 | 12 | 122243884 | 122724481 | 0.2269 | 0.5424 | NA | NA | NA | NA |
| WCM139_Z3_1 | 12 | 122726964 | 133810752 | −0.238 | −0.0135 | 1.0029 | 0.9784 | 1 | 1 |
| WCM139_Z3_1 | 13 | 19600515 | 36521571 | −0.1999 | 0.0333 | 1.0372 | 1.0095 | 1 | 1 |
| WCM139_Z3_1 | 13 | 36686097 | 40362474 | 0.3314 | 0.6631 | NA | NA | NA | NA |
| WCM139_Z3_1 | 13 | 41133822 | 44463003 | −0.2269 | 1.00E−04 | NA | NA | NA | NA |
| WCM139_Z3_1 | 13 | 44464314 | 48523122 | 0.2446 | 0.563 | 1.8693 | 1.0853 | 2 | 1 |
| WCM139_Z3_1 | 13 | 48523626 | 70281778 | −0.2532 | −0.0325 | 1.0129 | 0.9426 | 1 | 1 |
| WCM139_Z3_1 | 13 | 70293693 | 115089411 | −0.9337 | −0.9382 | 1.0075 | 0.0362 | 1 | 0 |
| WCM139_Z3_1 | 14 | 19377692 | 45639921 | −0.2115 | 0.0191 | 1.0445 | 0.9821 | 1 | 1 |
| WCM139_Z3_1 | 14 | 45642275 | 52188693 | 0.2459 | 0.5645 | 1.9467 | 1.0111 | 2 | 1 |
| WCM139_Z3_1 | 14 | 52192560 | 57684711 | 0.8459 | 1.2404 | 2.9158 | 1.8094 | 3 | 2 |
| WCM139_Z3_1 | 14 | 57686153 | 59113803 | 0.2798 | 0.6037 | NA | NA | NA | NA |
| WCM139_Z3_1 | 14 | 59730325 | 60113684 | 0.8961 | 1.2955 | NA | NA | NA | NA |
| WCM139_Z3_1 | 14 | 60193646 | 73440893 | 0.2601 | 0.5809 | 1.9509 | 1.0407 | 2 | 1 |
| WCM139_Z3_1 | 14 | 73441484 | 74370681 | 0.6875 | 1.0652 | NA | NA | NA | NA |
| WCM139_Z3_1 | 14 | 74371774 | 107283027 | 0.3006 | 0.6277 | 2.0363 | 1.0539 | 2 | 1 |
| WCM139_Z3_1 | 15 | 20170065 | 25485047 | −0.2754 | −0.06 | 0.9855 | 0.933 | 1 | 1 |
| WCM139_Z3_1 | 15 | 25486929 | 33905410 | 0.1326 | 0.4323 | 1.7359 | 0.9629 | 2 | 1 |
| WCM139_Z3_1 | 15 | 33916210 | 45983178 | −0.2235 | 0.0043 | 1.0203 | 0.9857 | 1 | 1 |
| WCM139_Z3_1 | 15 | 47873549 | 49867217 | 0.2563 | 0.5765 | NA | NA | NA | NA |
| WCM139_Z3_1 | 15 | 49869018 | 64962630 | −0.2183 | 0.0108 | 1.0547 | 0.9603 | 1 | 1 |
| WCM139_Z3_1 | 15 | 64966287 | 66161902 | 0.2683 | 0.5904 | 2.0695 | 0.9418 | 2 | 1 |
| WCM139_Z3_1 | 15 | 66169731 | 78211164 | −0.2197 | 0.009 | 1.0459 | 0.9665 | 1 | 1 |
| WCM139_Z3_1 | 15 | 78211598 | 78640285 | 0.3125 | 0.6414 | NA | NA | NA | NA |
| WCM139_Z3_1 | 15 | 78730689 | 80988319 | −0.2589 | −0.0395 | NA | NA | NA | NA |
| WCM139_Z3_1 | 15 | 81041941 | 83673685 | 0.22 | 0.5344 | 1.864 | 1.0327 | 2 | 1 |
| WCM139_Z3_1 | 15 | 83674420 | 87572062 | −0.2432 | −0.0201 | 1.057 | 0.9154 | 1 | 1 |
| WCM139_Z3_1 | 15 | 88420207 | 91145581 | 0.2874 | 0.6124 | 1.8939 | 1.1638 | 2 | 1 |
| WCM139_Z3_1 | 15 | 91147681 | 102514188 | −0.2244 | 0.0032 | 1.0529 | 0.9515 | 1 | 1 |
| WCM139_Z3_1 | 16 | 66926 | 396911 | 0.2808 | 0.6049 | NA | NA | NA | NA |
| WCM139_Z3_1 | 16 | 417421 | 1808983 | −0.2057 | 0.0263 | 1.1362 | 0.9006 | 1 | 1 |
| WCM139_Z3_1 | 16 | 1809990 | 2376459 | 0.2535 | 0.5734 | 1.9169 | 1.0591 | 2 | 1 |
| WCM139_Z3_1 | 16 | 2378556 | 2854443 | 0.7069 | 1.0868 | NA | NA | NA | NA |
| WCM139_Z3_1 | 16 | 2854940 | 3658959 | 0.3124 | 0.6413 | 2.1644 | 0.955 | 2 | 1 |
| WCM139_Z3_1 | 16 | 3705454 | 14403204 | −0.2125 | 0.0178 | 1.0835 | 0.9414 | 1 | 1 |
| WCM139_Z3_1 | 16 | 14530603 | 15493299 | 0.3674 | 0.7044 | NA | NA | NA | NA |
| WCM139_Z3_1 | 16 | 15494662 | 23417575 | −0.2236 | 0.0042 | 1.0178 | 0.9881 | 1 | 1 |
| WCM139_Z3_1 | 16 | 23424885 | 31161332 | 0.2332 | 0.5498 | 2.004 | 0.9238 | 2 | 1 |
| WCM139_Z3_1 | 16 | 31191534 | 33962083 | −0.2244 | 0.0033 | 1.0567 | 0.9478 | 1 | 1 |
| WCM139_Z3_1 | 16 | 34256804 | 49672775 | 0.4091 | 0.752 | 2.2694 | 1.0989 | 2 | 1 |
| WCM139_Z3_1 | 16 | 49764710 | 68338019 | −0.2292 | −0.0027 | 1.0228 | 0.9735 | 1 | 1 |
| WCM139_Z3_1 | 16 | 68344256 | 71660460 | −0.988 | −1.0176 | 0.9923 | −0.0044 | 1 | 0 |
| WCM139_Z3_1 | 16 | 71663281 | 72124583 | −0.2399 | −0.016 | NA | NA | NA | NA |
| WCM139_Z3_1 | 16 | 72130203 | 79998102 | −0.9915 | −1.0227 | 0.99 | −0.0056 | 1 | 0 |
| WCM139_Z3_1 | 16 | 80574703 | 81301694 | −0.2079 | 0.0235 | 2.0723 | −0.0394 | 2 | 0 |
| WCM139_Z3_1 | 16 | 81303866 | 90233622 | −0.9512 | −0.9636 | 0.9918 | 0.0337 | 1 | 0 |
| WCM139_Z3_1 | 17 | 131595 | 1198796 | −0.2013 | 0.0316 | NA | NA | NA | NA |
| WCM139_Z3_1 | 17 | 1201557 | 2324082 | 0.5995 | 0.967 | 2.9115 | 0.998 | 3 | 1 |
| WCM139_Z3_1 | 17 | 2341421 | 3592019 | 0.2022 | 0.5138 | 1.8766 | 0.9789 | 2 | 1 |
| WCM139_Z3_1 | 17 | 3592816 | 4145563 | 0.6304 | 1.0015 | 3.04 | 0.9643 | 3 | 1 |
| WCM139_Z3_1 | 17 | 4166914 | 7456905 | −0.2638 | −0.0456 | 0.9942 | 0.9436 | 1 | 1 |
| WCM139_Z3_1 | 17 | 7460163 | 8012560 | 0.1964 | 0.507 | 1.8814 | 0.9608 | 2 | 1 |
| WCM139_Z3_1 | 17 | 8013280 | 12656601 | −0.2555 | −0.0352 | 1.0291 | 0.9226 | 1 | 1 |
| WCM139_Z3_1 | 17 | 12659793 | 15154966 | 0.2204 | 0.5349 | NA | NA | NA | NA |
| WCM139_Z3_1 | 17 | 15162496 | 19555902 | −0.242 | −0.0186 | 1.0178 | 0.9567 | 1 | 1 |
| WCM139_Z3_1 | 17 | 19559771 | 20013850 | 0.2272 | 0.5428 | NA | NA | NA | NA |
| WCM139_Z3_1 | 17 | 20107768 | 27382139 | −0.2222 | 0.006 | 1.0933 | 0.915 | 1 | 1 |
| WCM139_Z3_1 | 17 | 27382899 | 29902503 | 0.232 | 0.5484 | 1.8578 | 1.067 | 2 | 1 |
| WCM139_Z3_1 | 17 | 29902649 | 35295109 | −0.2323 | −0.0065 | 1.0385 | 0.9524 | 1 | 1 |
| WCM139_Z3_1 | 17 | 35295511 | 35969400 | 0.1694 | 0.4755 | NA | NA | NA | NA |
| WCM139_Z3_1 | 17 | 35974350 | 37564678 | −0.2645 | −0.0465 | 1.0164 | 0.9202 | 1 | 1 |
| WCM139_Z3_1 | 17 | 37564803 | 40107322 | 0.2478 | 0.5667 | 2.1297 | 0.8326 | 2 | 1 |
| WCM139_Z3_1 | 17 | 40113427 | 44632696 | −0.2485 | −0.0266 | 1.0182 | 0.9453 | 1 | 1 |
| WCM139_Z3_1 | 17 | 44770354 | 45662923 | 0.2033 | 0.5151 | NA | NA | NA | NA |
| WCM139_Z3_1 | 17 | 45663749 | 46940405 | −0.219 | 0.0099 | NA | NA | NA | NA |
| WCM139_Z3_1 | 17 | 46941682 | 47700140 | 0.1891 | 0.4985 | NA | NA | NA | NA |
| WCM139_Z3_1 | 17 | 47778827 | 57352498 | −0.1996 | 0.0337 | 1.0576 | 0.9897 | 1 | 1 |
| WCM139_Z3_1 | 17 | 57430917 | 61021614 | 0.5114 | 0.868 | 2.0393 | 1.6109 | 2 | 2 |
| WCM139_Z3_1 | 17 | 61151371 | 72200289 | −0.2449 | −0.0221 | 1.0031 | 0.9665 | 1 | 1 |
| WCM139_Z3_1 | 17 | 72200355 | 76060954 | 0.2408 | 0.5586 | 1.9048 | 1.0408 | 2 | 1 |
| WCM139_Z3_1 | 17 | 76063871 | 79108177 | 0.6091 | 0.9777 | 3.0383 | 0.9005 | 3 | 1 |
| WCM139_Z3_1 | 17 | 79139473 | 79686863 | 1.2967 | 1.7291 | NA | NA | NA | NA |
| WCM139_Z3_1 | 17 | 79766901 | 81083455 | 0.6138 | 0.983 | 2.9539 | 0.9993 | 3 | 1 |
| WCM139_Z3_1 | 18 | 48213 | 9275534 | −0.2326 | −0.007 | 1.0333 | 0.9571 | 1 | 1 |
| WCM139_Z3_1 | 18 | 9279602 | 12948193 | 0.8095 | 1.2003 | 3.5894 | 1.0063 | 4 | 1 |
| WCM139_Z3_1 | 18 | 12963341 | 25727674 | −0.2176 | 0.0116 | NA | NA | NA | NA |
| WCM139_Z3_1 | 18 | 27878913 | 30093503 | 0.5305 | 0.8895 | NA | NA | NA | NA |
| WCM139_Z3_1 | 18 | 30093721 | 43796059 | −0.22 | 0.0086 | 1.0271 | 0.9849 | 1 | 1 |
| WCM139_Z3_1 | 18 | 43796338 | 46343675 | 0.2566 | 0.5769 | NA | NA | NA | NA |
| WCM139_Z3_1 | 18 | 46383964 | 55398796 | −0.1932 | 0.0416 | 1.071 | 0.9875 | 1 | 1 |
| WCM139_Z3_1 | 18 | 55398922 | 59739939 | 0.287 | 0.612 | 2.2952 | 0.7615 | 2 | 1 |
| WCM139_Z3_1 | 18 | 59742023 | 60572499 | −0.2496 | −0.028 | NA | NA | NA | NA |
| WCM139_Z3_1 | 18 | 60582155 | 64235922 | 0.2482 | 0.5672 | NA | NA | NA | NA |
| WCM139_Z3_1 | 18 | 64239251 | 78005224 | −0.2377 | −0.0133 | 1.0791 | 0.9026 | 1 | 1 |
| WCM139_Z3_1 | 19 | 281467 | 1650211 | −0.3001 | −0.0908 | 0.939 | 0.939 | 1 | 1 |
| WCM139_Z3_1 | 19 | 1753670 | 2807738 | 0.2187 | 0.5329 | 1.9261 | 0.9677 | 2 | 1 |
| WCM139_Z3_1 | 19 | 2808388 | 14302221 | −0.2503 | −0.0288 | 0.9985 | 0.962 | 1 | 1 |
| WCM139_Z3_1 | 19 | 14492335 | 16799176 | 0.2484 | 0.5674 | 1.9624 | 1.0012 | 2 | 1 |
| WCM139_Z3_1 | 19 | 16842052 | 21607380 | −0.2301 | −0.0038 | 1.0306 | 0.9641 | 1 | 1 |
| WCM139_Z3_1 | 19 | 21607737 | 30934589 | 0.4229 | 0.7677 | 1.8169 | 1.5883 | 2 | 2 |
| WCM139_Z3_1 | 19 | 30934880 | 42753380 | −0.2546 | −0.0342 | 0.9996 | 0.9536 | 1 | 1 |
| WCM139_Z3_1 | 19 | 42753466 | 46214570 | 0.2192 | 0.5335 | 1.905 | 0.9899 | 2 | 1 |
| WCM139_Z3_1 | 19 | 46214902 | 51961604 | −0.258 | −0.0384 | 0.9931 | 0.9544 | 1 | 1 |
| WCM139_Z3_1 | 19 | 51979838 | 52579362 | 0.2668 | 0.5887 | 1.9141 | 1.0936 | 2 | 1 |
| WCM139_Z3_1 | 19 | 52580310 | 53518635 | −0.1874 | 0.0487 | 1.0788 | 0.9899 | 1 | 1 |
| WCM139_Z3_1 | 19 | 53553335 | 54973533 | 0.2251 | 0.5404 | 1.9796 | 0.9291 | 2 | 1 |
| WCM139_Z3_1 | 19 | 55087523 | 56226552 | 0.599 | 0.9665 | 2.8601 | 1.048 | 3 | 1 |
| WCM139_Z3_1 | 19 | 56228114 | 57081723 | 0.2123 | 0.5255 | 1.8613 | 1.0176 | 2 | 1 |
| WCM139_Z3_1 | 19 | 57085769 | 59083929 | −0.2117 | 0.0189 | 1.0531 | 0.9733 | 1 | 1 |
| WCM139_Z3_1 | 2 | 41612 | 24107699 | −0.2134 | 0.0168 | 1.0167 | 1.0067 | 1 | 1 |
| WCM139_Z3_1 | 2 | 24108669 | 42552376 | 0.2417 | 0.5596 | 1.9146 | 1.0331 | 2 | 1 |
| WCM139_Z3_1 | 2 | 42552660 | 44599932 | 0.5643 | 0.9276 | 1.9555 | 1.8487 | 2 | 2 |
| WCM139_Z3_1 | 2 | 44617429 | 45812746 | 0.1997 | 0.5109 | NA | NA | NA | NA |
| WCM139_Z3_1 | 2 | 45826631 | 74146645 | −0.2272 | −2.00E−04 | 1.0233 | 0.9764 | 1 | 1 |
| WCM139_Z3_1 | 2 | 74154158 | 75425703 | 0.2268 | 0.5424 | NA | NA | NA | NA |
| WCM139_Z3_1 | 2 | 75425992 | 100360602 | −0.2241 | 0.0036 | 1.0532 | 0.9518 | 1 | 1 |
| WCM139_Z3_1 | 2 | 100368683 | 102644785 | 0.5696 | 0.9335 | NA | NA | NA | NA |
| WCM139_Z3_1 | 2 | 102774221 | 108608649 | 0.0399 | 0.3229 | 1.4202 | 1.0815 | 1 | 1 |
| WCM139_Z3_1 | 2 | 108609470 | 120187509 | −0.2317 | −0.0058 | 1.0181 | 0.9738 | 1 | 1 |
| WCM139_Z3_1 | 2 | 120187954 | 122522850 | 0.2307 | 0.5469 | NA | NA | NA | NA |
| WCM139_Z3_1 | 2 | 124771732 | 129076090 | −0.8954 | −0.883 | 1.1163 | −0.0318 | 1 | 0 |
| WCM139_Z3_1 | 2 | 130738450 | 133472462 | −0.2788 | −0.0642 | 1.939 | −0.0261 | 2 | 0 |
| WCM139_Z3_1 | 2 | 133483232 | 150432976 | −0.9456 | −0.9556 | 0.9988 | 0.0325 | 1 | 0 |
| WCM139_Z3_1 | 2 | 150436157 | 153576426 | −0.2417 | −0.0181 | 1.9334 | 0.0416 | 2 | 0 |
| WCM139_Z3_1 | 2 | 153577013 | 167760399 | −0.9482 | −0.9593 | 1.043 | −0.0144 | 1 | 0 |
| WCM139_Z3_1 | 2 | 167992429 | 171503439 | −0.2314 | −0.0054 | 2.0031 | −0.0106 | 2 | 0 |
| WCM139_Z3_1 | 2 | 171508150 | 193059210 | −0.9495 | −0.9612 | 1.0314 | −0.0041 | 1 | 0 |
| WCM139_Z3_1 | 2 | 196544880 | 202154564 | −0.2252 | 0.0022 | 1.9707 | 0.0324 | 2 | 0 |
| WCM139_Z3_1 | 2 | 202172299 | 209436747 | −0.9473 | −0.958 | 1.0378 | −0.0083 | 1 | 0 |
| WCM139_Z3_1 | 2 | 210518140 | 212652776 | −0.0444 | 0.2223 | NA | NA | NA | NA |
| WCM139_Z3_1 | 2 | 212812152 | 218941184 | −0.8432 | −0.8088 | 1.2272 | −0.0856 | 1 | 0 |
| WCM139_Z3_1 | 2 | 218947956 | 219890870 | 0.2689 | 0.5911 | 1.9329 | 1.0798 | 2 | 1 |
| WCM139_Z3_1 | 2 | 219892233 | 220497702 | 0.901 | 1.3008 | 3.8857 | 1.0417 | 4 | 1 |
| WCM139_Z3_1 | 2 | 220498120 | 220506402 | 0.4966 | 0.8512 | NA | NA | NA | NA |
| WCM139_Z3_1 | 2 | 220521277 | 234294615 | −0.9522 | −0.9651 | 0.9902 | 0.0343 | 1 | 0 |
| WCM139_Z3_1 | 2 | 234296984 | 234851280 | −0.2537 | −0.033 | NA | NA | NA | NA |
| WCM139_Z3_1 | 2 | 234855801 | 242946597 | −0.9464 | −0.9567 | 1.0341 | −0.0036 | 1 | 0 |
| WCM139_Z3_1 | 20 | 68375 | 2084004 | −0.2565 | −0.0365 | 1.0125 | 0.9375 | 1 | 1 |
| WCM139_Z3_1 | 20 | 2097457 | 2473428 | 0.2888 | 0.6141 | NA | NA | NA | NA |
| WCM139_Z3_1 | 20 | 2474188 | 5170848 | −0.2183 | 0.0107 | 1.0385 | 0.9764 | 1 | 1 |
| WCM139_Z3_1 | 20 | 5261132 | 8737771 | 0.3165 | 0.646 | 1.7988 | 1.3309 | 2 | 1 |
| WCM139_Z3_1 | 20 | 8741093 | 29632635 | −0.2005 | 0.0326 | 1.048 | 0.9977 | 1 | 1 |
| WCM139_Z3_1 | 20 | 29845492 | 34858541 | 0.2733 | 0.5962 | 2.0076 | 1.0158 | 2 | 1 |
| WCM139_Z3_1 | 20 | 35060189 | 35802464 | 0.7892 | 1.1779 | NA | NA | NA | NA |
| WCM139_Z3_1 | 20 | 35807924 | 45362401 | −0.2274 | −5.00E−04 | 1.0355 | 0.9639 | 1 | 1 |
| WCM139_Z3_1 | 20 | 45618638 | 62904925 | 0.2465 | 0.5652 | 1.9939 | 0.9654 | 2 | 1 |
| WCM139_Z3_1 | 21 | 9826203 | 36422352 | −0.0425 | 0.2246 | 1.4336 | 0.9033 | 1 | 1 |
| WCM139_Z3_1 | 21 | 37093104 | 39947586 | 0.4017 | 0.7436 | 2.1096 | 1.2391 | 2 | 1 |
| WCM139_Z3_1 | 21 | 40177454 | 44949723 | −0.2397 | −0.0156 | 1.0235 | 0.9549 | 1 | 1 |
| WCM139_Z3_1 | 21 | 45012126 | 45404974 | 0.2819 | 0.6061 | NA | NA | NA | NA |
| WCM139_Z3_1 | 21 | 45432414 | 46101782 | −0.2734 | −0.0576 | 1.0362 | 0.8856 | 1 | 1 |
| WCM139_Z3_1 | 21 | 46226838 | 47316244 | 0.2161 | 0.5299 | 1.9037 | 0.984 | 2 | 1 |
| WCM139_Z3_1 | 21 | 47319512 | 48084277 | −0.2579 | −0.0382 | 1.0439 | 0.9038 | 1 | 1 |
| WCM139_Z3_1 | 22 | 16287443 | 23504959 | −0.226 | 0.0013 | 1.1244 | 0.8773 | 1 | 1 |
| WCM139_Z3_1 | 22 | 23523334 | 26423602 | 0.4974 | 0.8521 | 2.5538 | 1.0566 | 3 | 1 |
| WCM139_Z3_1 | 22 | 26565683 | 32628995 | −0.2492 | −0.0274 | 0.9994 | 0.9629 | 1 | 1 |
| WCM139_Z3_1 | 22 | 32630966 | 37771407 | 0.2521 | 0.5717 | 2.0059 | 0.9666 | 2 | 1 |
| WCM139_Z3_1 | 22 | 37771437 | 42388764 | −0.2674 | −0.05 | 0.9947 | 0.9371 | 1 | 1 |
| WCM139_Z3_1 | 22 | 42390346 | 43253126 | 0.2296 | 0.5456 | NA | NA | NA | NA |
| WCM139_Z3_1 | 22 | 43254425 | 51219138 | −0.2435 | −0.0204 | 1.0043 | 0.9677 | 1 | 1 |
| WCM139_Z3_1 | 3 | 239451 | 9408623 | 0.1793 | 0.4871 | 1.9105 | 0.8928 | 2 | 1 |
| WCM139_Z3_1 | 3 | 9412884 | 11078549 | −0.215 | 0.0148 | 1.0676 | 0.953 | 1 | 1 |
| WCM139_Z3_1 | 3 | 11300891 | 13921258 | 0.2673 | 0.5893 | 1.9914 | 1.0176 | 2 | 1 |
| WCM139_Z3_1 | 3 | 14105751 | 36422183 | −0.2249 | 0.0026 | 1.0426 | 0.961 | 1 | 1 |
| WCM139_Z3_1 | 3 | 36484996 | 38519424 | 0.2403 | 0.558 | 1.7777 | 1.1667 | 2 | 1 |
| WCM139_Z3_1 | 3 | 38519740 | 57835500 | −0.207 | 0.0247 | 1.036 | 0.9985 | 1 | 1 |
| WCM139_Z3_1 | 3 | 57843458 | 58303759 | 0.2387 | 0.5562 | NA | NA | NA | NA |
| WCM139_Z3_1 | 3 | 58318751 | 110611201 | −0.2054 | 0.0266 | 1.05 | 0.9873 | 1 | 1 |
| WCM139_Z3_1 | 3 | 110611399 | 114833040 | 0.2778 | 0.6014 | 2.0883 | 0.9461 | 2 | 1 |
| WCM139_Z3_1 | 3 | 115382658 | 121168251 | −0.2152 | 0.0146 | 1.04 | 0.9803 | 1 | 1 |
| WCM139_Z3_1 | 3 | 121178933 | 123987618 | 0.2895 | 0.6149 | 2.0612 | 1.0016 | 2 | 1 |
| WCM139_Z3_1 | 3 | 123987946 | 127642943 | −0.2213 | 0.0071 | 1.0198 | 0.99 | 1 | 1 |
| WCM139_Z3_1 | 3 | 127646773 | 127981029 | 0.1976 | 0.5084 | NA | NA | NA | NA |
| WCM139_Z3_1 | 3 | 127983495 | 129389918 | 0.6334 | 1.0049 | NA | NA | NA | NA |
| WCM139_Z3_1 | 3 | 129390084 | 132075707 | 0.2703 | 0.5927 | 2.0309 | 0.9853 | 2 | 1 |
| WCM139_Z3_1 | 3 | 132086650 | 135809488 | −0.2099 | 0.0211 | 1.0409 | 0.9885 | 1 | 1 |
| WCM139_Z3_1 | 3 | 135820954 | 138382851 | 0.3732 | 0.711 | NA | NA | NA | NA |
| WCM139_Z3_1 | 3 | 138383917 | 141457251 | −0.2254 | 0.0019 | NA | NA | NA | NA |
| WCM139_Z3_1 | 3 | 141461508 | 149678767 | 0.2542 | 0.5741 | 1.7714 | 1.2061 | 2 | 1 |
| WCM139_Z3_1 | 3 | 149678839 | 178525219 | −0.2009 | 0.0322 | 1.0791 | 0.966 | 1 | 1 |
| WCM139_Z3_1 | 3 | 178543436 | 180703742 | 0.2171 | 0.5311 | NA | NA | NA | NA |
| WCM139_Z3_1 | 3 | 180704759 | 183604613 | −0.1978 | 0.036 | NA | NA | NA | NA |
| WCM139_Z3_1 | 3 | 183639133 | 183993980 | 0.3237 | 0.6543 | 1.8559 | 1.2917 | 2 | 1 |
| WCM139_Z3_1 | 3 | 183994494 | 184580798 | 1.113 | 1.5315 | 4.8067 | 0.975 | 5 | 1 |
| WCM139_Z3_1 | 3 | 184585821 | 185774947 | 0.2285 | 0.5443 | NA | NA | NA | NA |
| WCM139_Z3_1 | 3 | 185775243 | 194177886 | −0.1982 | 0.0355 | 1.04 | 1.0098 | 1 | 1 |
| WCM139_Z3_1 | 3 | 194180267 | 196454862 | 0.2554 | 0.5755 | 1.8921 | 1.0884 | 2 | 1 |
| WCM139_Z3_1 | 3 | 196455044 | 197894726 | −0.1707 | 0.0692 | NA | NA | NA | NA |
| WCM139_Z3_1 | 4 | 59327 | 36115795 | −0.2176 | 0.0116 | 1.0243 | 0.9919 | 1 | 1 |
| WCM139_Z3_1 | 4 | 36118684 | 41496615 | 0.3382 | 0.6709 | 2.0907 | 1.0934 | 2 | 1 |
| WCM139_Z3_1 | 4 | 41526470 | 52938245 | −0.2604 | −0.0413 | 1.0333 | 0.9102 | 1 | 1 |
| WCM139_Z3_1 | 4 | 52943010 | 57976295 | 0.1303 | 0.4297 | 1.7296 | 0.9642 | 2 | 1 |
| WCM139_Z3_1 | 4 | 57976467 | 71471920 | −0.237 | −0.0124 | 1.0785 | 0.9044 | 1 | 1 |
| WCM139_Z3_1 | 4 | 71472184 | 72121069 | 0.1882 | 0.4974 | NA | NA | NA | NA |
| WCM139_Z3_1 | 4 | 72205092 | 72649700 | 2.3897 | 2.8753 | NA | NA | NA | NA |
| WCM139_Z3_1 | 4 | 72669661 | 74276042 | 0.8026 | 1.1927 | NA | NA | NA | NA |
| WCM139_Z3_1 | 4 | 74277821 | 78532179 | 0.2467 | 0.5654 | 1.9427 | 1.0169 | 2 | 1 |
| WCM139_Z3_1 | 4 | 78647310 | 80828618 | 0.8996 | 1.2993 | NA | NA | NA | NA |
| WCM139_Z3_1 | 4 | 80898758 | 107956693 | −0.2228 | 0.0051 | 1.058 | 0.9491 | 1 | 1 |
| WCM139_Z3_1 | 4 | 108049099 | 110638739 | 0.2936 | 0.6196 | NA | NA | NA | NA |
| WCM139_Z3_1 | 4 | 110650812 | 128544639 | −0.2394 | −0.0153 | 1.0347 | 0.9442 | 1 | 1 |
| WCM139_Z3_1 | 4 | 128554268 | 130032843 | 0.2383 | 0.5557 | NA | NA | NA | NA |
| WCM139_Z3_1 | 4 | 133357010 | 153273817 | −0.2245 | 0.003 | 1.0565 | 0.9477 | 1 | 1 |
| WCM139_Z3_1 | 4 | 153303348 | 155665486 | 0.2998 | 0.6267 | 2.0581 | 1.03 | 2 | 1 |
| WCM139_Z3_1 | 4 | 155665792 | 190426502 | −0.2121 | 0.0184 | 1.029 | 0.9966 | 1 | 1 |
| WCM139_Z3_1 | 5 | 140663 | 13841142 | −0.2252 | 0.0023 | 1.0189 | 0.9843 | 1 | 1 |
| WCM139_Z3_1 | 5 | 13841860 | 14741984 | 0.3102 | 0.6388 | NA | NA | NA | NA |
| WCM139_Z3_1 | 5 | 14746068 | 78077787 | −0.1632 | 0.0784 | 1.0842 | 1.0274 | 1 | 1 |
| WCM139_Z3_1 | 5 | 78111939 | 78532658 | 0.311 | 0.6396 | 2.0473 | 1.0686 | 2 | 1 |
| WCM139_Z3_1 | 5 | 78532842 | 111071153 | −0.1967 | 0.0373 | 1.0476 | 1.0047 | 1 | 1 |
| WCM139_Z3_1 | 5 | 111091470 | 113831705 | 0.2595 | 0.5802 | 1.7649 | 1.2252 | 2 | 1 |
| WCM139_Z3_1 | 5 | 113831849 | 118456823 | −0.188 | 0.048 | NA | NA | NA | NA |
| WCM139_Z3_1 | 5 | 118464968 | 124079877 | 0.3747 | 0.7127 | NA | NA | NA | NA |
| WCM139_Z3_1 | 5 | 124080276 | 139819746 | −0.1983 | 0.0354 | 1.0475 | 1.0022 | 1 | 1 |
| WCM139_Z3_1 | 5 | 139820727 | 141059563 | 0.1633 | 0.4683 | 1.8851 | 0.8819 | 2 | 1 |
| WCM139_Z3_1 | 5 | 141059868 | 148577925 | −0.2082 | 0.0232 | 1.0712 | 0.9612 | 1 | 1 |
| WCM139_Z3_1 | 5 | 148578655 | 149151698 | 0.3244 | 0.6551 | NA | NA | NA | NA |
| WCM139_Z3_1 | 5 | 149200145 | 154169933 | −0.1856 | 0.0509 | 1.0715 | 1.0004 | 1 | 1 |
| WCM139_Z3_1 | 5 | 154170269 | 157240229 | 0.2537 | 0.5735 | 1.9705 | 1.0058 | 2 | 1 |
| WCM139_Z3_1 | 5 | 157241277 | 160016649 | 1.0334 | 1.4452 | NA | NA | NA | NA |
| WCM139_Z3_1 | 5 | 160018106 | 166802458 | 0.4344 | 0.7807 | NA | NA | NA | NA |
| WCM139_Z3_1 | 5 | 167182138 | 168310283 | 0.8902 | 1.289 | NA | NA | NA | NA |
| WCM139_Z3_1 | 5 | 168620527 | 171533788 | 0.2467 | 0.5654 | 1.9324 | 1.0272 | 2 | 1 |
| WCM139_Z3_1 | 5 | 171534801 | 174178774 | 0.5535 | 0.9154 | NA | NA | NA | NA |
| WCM139_Z3_1 | 5 | 174868920 | 180687773 | 0.2202 | 0.5347 | 1.9335 | 0.9639 | 2 | 1 |
| WCM139_Z3_1 | 6 | 203466 | 29231274 | −0.2095 | 0.0216 | 1.0313 | 0.9988 | 1 | 1 |
| WCM139_Z3_1 | 6 | 29407845 | 31324336 | 0.3211 | 0.6513 | 2.049 | 1.0923 | 2 | 1 |
| WCM139_Z3_1 | 6 | 31540784 | 31685563 | 1.0842 | 1.5003 | NA | NA | NA | NA |
| WCM139_Z3_1 | 6 | 31696497 | 34214830 | 0.232 | 0.5484 | 1.8579 | 1.0671 | 2 | 1 |
| WCM139_Z3_1 | 6 | 34215247 | 36807104 | −0.2216 | 0.0067 | 1.0195 | 0.9898 | 1 | 1 |
| WCM139_Z3_1 | 6 | 36823622 | 38370599 | 0.2237 | 0.5388 | 1.8645 | 1.0411 | 2 | 1 |
| WCM139_Z3_1 | 6 | 38455460 | 39835367 | 0.6572 | 1.0315 | 3.0399 | 1.0483 | 3 | 1 |
| WCM139_Z3_1 | 6 | 39835602 | 40359772 | 0.1888 | 0.4981 | NA | NA | NA | NA |
| WCM139_Z3_1 | 6 | 40359947 | 43128579 | −0.2096 | 0.0214 | 1.035 | 0.995 | 1 | 1 |
| WCM139_Z3_1 | 6 | 43139725 | 43336706 | 0.3037 | 0.6313 | NA | NA | NA | NA |
| WCM139_Z3_1 | 6 | 43395781 | 43752354 | 0.836 | 1.2295 | NA | NA | NA | NA |
| WCM139_Z3_1 | 6 | 43968380 | 46047721 | 0.3703 | 0.7077 | NA | NA | NA | NA |
| WCM139_Z3_1 | 6 | 46107610 | 51908440 | −0.2232 | 0.0047 | 1.0677 | 0.9389 | 1 | 1 |
| WCM139_Z3_1 | 6 | 51909798 | 54219399 | 0.2557 | 0.5759 | 1.9719 | 1.0093 | 2 | 1 |
| WCM139_Z3_1 | 6 | 54254650 | 112443287 | −0.2216 | 0.0066 | 1.1521 | 0.8571 | 1 | 1 |
| WCM139_Z3_1 | 6 | 112460366 | 118635317 | 0.2415 | 0.5594 | 1.8341 | 1.1132 | 2 | 1 |
| WCM139_Z3_1 | 6 | 118786608 | 143095711 | −0.201 | 0.0321 | 1.069 | 0.976 | 1 | 1 |
| WCM139_Z3_1 | 6 | 143382110 | 146755515 | 0.2493 | 0.5685 | 1.8402 | 1.1257 | 2 | 1 |
| WCM139_Z3_1 | 6 | 146755818 | 151362942 | −0.2074 | 0.0242 | 1.0319 | 1.0019 | 1 | 1 |
| WCM139_Z3_1 | 6 | 151413621 | 155764437 | 0.2641 | 0.5856 | 1.9761 | 1.0252 | 2 | 1 |
| WCM139_Z3_1 | 6 | 155774570 | 170893407 | −0.2364 | −0.0116 | 1.0128 | 0.9711 | 1 | 1 |
| WCM139_Z3_1 | 7 | 193451 | 37934083 | −0.9728 | −0.9953 | 0.9875 | 0.0157 | 1 | 0 |
| WCM139_Z3_1 | 7 | 37936681 | 57529300 | −0.2159 | 0.0137 | 1.0307 | 0.9884 | 1 | 1 |
| WCM139_Z3_1 | 7 | 57529593 | 92404119 | 0.2572 | 0.5776 | 1.9691 | 1.0158 | 2 | 1 |
| WCM139_Z3_1 | 7 | 92462599 | 98581969 | 0.8287 | 1.2214 | 3.8433 | 0.8202 | 4 | 1 |
| WCM139_Z3_1 | 7 | 98582587 | 99590023 | 0.3006 | 0.6276 | 1.9366 | 1.1535 | 2 | 1 |
| WCM139_Z3_1 | 7 | 99621300 | 100410718 | 0.9849 | 1.3925 | 4.0867 | 1.1639 | 4 | 1 |
| WCM139_Z3_1 | 7 | 100411638 | 100683439 | 0.661 | 1.0357 | 2.8406 | 1.2596 | 3 | 1 |
| WCM139_Z3_1 | 7 | 100683721 | 105132022 | 0.8834 | 1.2815 | 3.9477 | 0.9143 | 4 | 1 |
| WCM139_Z3_1 | 7 | 105135645 | 134576402 | 0.2623 | 0.5835 | 1.9699 | 1.0271 | 2 | 1 |
| WCM139_Z3_1 | 7 | 134605465 | 135255861 | 0.9346 | 1.3375 | NA | NA | NA | NA |
| WCM139_Z3_1 | 7 | 135258537 | 142760171 | 0.3345 | 0.6667 | 2.0211 | 1.1538 | 2 | 1 |
| WCM139_Z3_1 | 7 | 142829266 | 150069209 | −0.2492 | −0.0275 | 1.9695 | −0.0072 | 2 | 0 |
| WCM139_Z3_1 | 7 | 150069438 | 158937066 | −0.9626 | −0.9803 | 1.0229 | −0.0092 | 1 | 0 |
| WCM139_Z3_1 | 8 | 190908 | 677159 | 0.3452 | 0.6789 | NA | NA | NA | NA |
| WCM139_Z3_1 | 8 | 681156 | 3253723 | 0.6077 | 0.9762 | NA | NA | NA | NA |
| WCM139_Z3_1 | 8 | 3256976 | 33347834 | 0.2465 | 0.5653 | 1.9802 | 0.9791 | 2 | 1 |
| WCM139_Z3_1 | 8 | 33353077 | 37791982 | 0.8833 | 1.2814 | 3.9073 | 0.9542 | 4 | 1 |
| WCM139_Z3_1 | 8 | 37792528 | 41166670 | 0.2617 | 0.5828 | NA | NA | NA | NA |
| WCM139_Z3_1 | 8 | 41293113 | 51617255 | 0.7505 | 1.1351 | 2.3835 | 2.0091 | 2 | 2 |
| WCM139_Z3_1 | 8 | 51621461 | 59059865 | 0.2658 | 0.5876 | 1.9753 | 1.0302 | 2 | 1 |
| WCM139_Z3_1 | 8 | 59324028 | 66963858 | 0.8344 | 1.2277 | NA | NA | NA | NA |
| WCM139_Z3_1 | 8 | 66989039 | 73157129 | 1.225 | 1.6522 | 5.2236 | 1.0624 | 5 | 1 |
| WCM139_Z3_1 | 8 | 73480129 | 81605342 | 0.8303 | 1.2232 | 3.6115 | 1.0577 | 4 | 1 |
| WCM139_Z3_1 | 8 | 81733706 | 94827553 | 0.2464 | 0.565 | 1.9628 | 0.9961 | 2 | 1 |
| WCM139_Z3_1 | 8 | 94928335 | 97621725 | 0.9168 | 1.3181 | NA | NA | NA | NA |
| WCM139_Z3_1 | 8 | 97797247 | 114627762 | 0.2671 | 0.5891 | 1.9094 | 1.0991 | 2 | 1 |
| WCM139_Z3_1 | 8 | 116426469 | 125877519 | 0.9125 | 1.3134 | 4.0191 | 0.9512 | 4 | 1 |
| WCM139_Z3_1 | 8 | 125987895 | 146279507 | 0.2715 | 0.5941 | 1.9822 | 1.0369 | 2 | 1 |
| WCM139_Z3_1 | 9 | 17083 | 93376085 | −0.218 | 0.0111 | 1.0338 | 0.9816 | 1 | 1 |
| WCM139_Z3_1 | 9 | 93606196 | 96326824 | 0.565 | 0.9283 | 1.9479 | 1.8581 | 2 | 2 |
| WCM139_Z3_1 | 9 | 96357172 | 129961296 | −0.2288 | −0.0022 | 1.0207 | 0.9763 | 1 | 1 |
| WCM139_Z3_1 | 9 | 129972982 | 134165505 | 0.2485 | 0.5676 | 1.9919 | 0.9722 | 2 | 1 |
| WCM139_Z3_1 | 9 | 134165753 | 136726500 | 0.6488 | 1.0221 | 3.0662 | 0.9956 | 3 | 1 |
| WCM139_Z3_1 | 9 | 136804303 | 139221397 | 0.2236 | 0.5387 | 1.8947 | 1.0106 | 2 | 1 |
| WCM139_Z3_1 | 9 | 139221680 | 141111414 | −0.2168 | 0.0126 | 1.0262 | 0.9914 | 1 | 1 |
| WCM139_Z3_1 | X | 2707682 | 15536060 | 0.4279 | 0.7734 | NA | NA | NA | NA |
| WCM139_Z3_1 | X | 15540585 | 21959443 | −0.212 | 0.0184 | NA | NA | NA | NA |
| WCM139_Z3_1 | X | 21985429 | 22186450 | 0.672 | 1.048 | NA | NA | NA | NA |
| WCM139_Z3_1 | X | 22196409 | 48435890 | −0.1521 | 0.0919 | NA | NA | NA | NA |
| WCM139_Z3_1 | X | 48542706 | 48979985 | 0.5865 | 0.9524 | NA | NA | NA | NA |
| WCM139_Z3_1 | X | 49021073 | 64771929 | −0.3307 | −0.1291 | NA | NA | NA | NA |
| WCM139_Z3_1 | X | 64949319 | 67751772 | 0.8931 | 1.2922 | NA | NA | NA | NA |
| WCM139_Z3_1 | X | 67867733 | 83141570 | −0.2446 | −0.0218 | NA | NA | NA | NA |
| WCM139_Z3_1 | X | 83319369 | 83480794 | 0.6884 | 1.0662 | NA | NA | NA | NA |
| WCM139_Z3_1 | X | 83581254 | 96396740 | −0.2765 | −0.0614 | NA | NA | NA | NA |
| WCM139_Z3_1 | X | 96603225 | 100494055 | 0.5952 | 0.9622 | NA | NA | NA | NA |
| WCM139_Z3_1 | X | 100496661 | 108926521 | −0.2085 | 0.0228 | NA | NA | NA | NA |
| WCM139_Z3_1 | X | 109247203 | 118513924 | 0.5005 | 0.8557 | NA | NA | NA | NA |
| WCM139_Z3_1 | X | 118540552 | 132458553 | −0.2689 | −0.0519 | NA | NA | NA | NA |
| WCM139_Z3_1 | X | 132473353 | 135047187 | 0.497 | 0.8517 | NA | NA | NA | NA |
| WCM139_Z3_1 | X | 135049578 | 154509348 | −0.2643 | −0.0462 | NA | NA | NA | NA |
| WCM142 | 1 | 14930 | 24859663 | 0.0171 | 0.1212 | 1.1883 | 0.9869 | 1 | 1 |
| WCM142 | 1 | 24861593 | 151258901 | −0.0541 | −1.0547 | 0.4814 | 0.4814 | 0 | 0 |
| WCM142 | 1 | 151259562 | 249214018 | 0.0466 | 0.431 | 1.3482 | 1.3482 | 1 | 1 |
| WCM142 | 10 | 226048 | 135477898 | −0.0067 | −0.1815 | 0.8818 | 0.8818 | 1 | 1 |
| WCM142 | 11 | 193096 | 134605906 | 0.0412 | 0.378 | 1.2995 | 1.2995 | 1 | 1 |
| WCM142 | 12 | 148427 | 133810703 | 0.0034 | −0.0459 | 0.9687 | 0.9687 | 1 | 1 |
| WCM142 | 13 | 19041992 | 115089411 | −0.0387 | −0.7135 | 0.6098 | 0.6098 | 1 | 1 |
| WCM142 | 14 | 19110343 | 107283255 | 0.0175 | 0.1261 | 1.0913 | 1.0913 | 1 | 1 |
| WCM142 | 15 | 20083896 | 102514200 | 0.0053 | −0.0219 | 0.9849 | 0.9849 | 1 | 1 |
| WCM142 | 16 | 64401 | 90168646 | −4.00E−04 | −0.0947 | 0.9365 | 0.9365 | 1 | 1 |
| WCM142 | 17 | 131595 | 1183426 | 0.0216 | 0.1729 | 1.3799 | 0.8748 | 1 | 1 |
| WCM142 | 17 | 1183637 | 8457234 | −0.1189 | NA | −0.0443 | −0.0443 | 0 | 0 |
| WCM142 | 17 | 8464687 | 81187891 | 0.0103 | 0.041 | 1.0289 | 1.0289 | 1 | 1 |
| WCM142 | 18 | 47610 | 78005210 | −0.0637 | −1.3143 | 0.4021 | 0.4021 | 0 | 0 |
| WCM142 | 19 | 105041 | 59093696 | 0.0235 | 0.1938 | 1.1438 | 1.1438 | 1 | 1 |
| WCM142 | 2 | 41620 | 243037022 | 0.0026 | −0.0557 | 0.9621 | 0.9621 | 1 | 1 |
| WCM142 | 20 | 68346 | 62934572 | 0.0234 | 0.1926 | 1.1428 | 1.1428 | 1 | 1 |
| WCM142 | 21 | 9590271 | 48084142 | 0.0205 | 0.1601 | 1.1174 | 1.1174 | 1 | 1 |
| WCM142 | 22 | 16159016 | 51219138 | 0.0481 | 0.445 | 1.3613 | 1.3613 | 1 | 1 |
| WCM142 | 3 | 239530 | 197896798 | 0.0243 | 0.2022 | 1.1505 | 1.1505 | 1 | 1 |
| WCM142 | 4 | 53423 | 190878508 | 0.0058 | −0.0154 | 0.9894 | 0.9894 | 1 | 1 |
| WCM142 | 5 | 90680 | 180687804 | −0.0532 | −1.0313 | 0.4893 | 0.4893 | 0 | 0 |
| WCM142 | 6 | 203475 | 170892848 | 0.0012 | −0.0741 | 0.95 | 0.95 | 1 | 1 |
| WCM142 | 7 | 193171 | 158937227 | 0.0505 | 0.4673 | 1.3825 | 1.3825 | 1 | 1 |
| WCM142 | 8 | 163432 | 48689570 | −0.0482 | −0.913 | 0.5311 | 0.5311 | 1 | 1 |
| WCM142 | 8 | 48690289 | 145948719 | 0.1067 | 0.9219 | 1.8946 | 1.8946 | 2 | 2 |
| WCM142 | 8 | 145979138 | 146279431 | −0.0665 | −1.3993 | NA | NA | NA | NA |
| WCM142 | 9 | 14893 | 141107519 | 0.0502 | 0.4643 | 1.3796 | 1.3796 | 1 | 1 |
| WCM142 | X | 2700145 | 154493609 | 0.0364 | 0.3306 | NA | NA | NA | NA |
| WCM14-TP1 | 1 | 762088 | 108724658 | −0.1074 | 0.8827 | 2.3593 | 1.3285 | 2 | 1 |
| WCM14-TP1 | 1 | 108728589 | 111216659 | −0.6427 | 0.1247 | 1.9861 | 0.1944 | 2 | 0 |
| WCM14-TP1 | 1 | 111217007 | 193099372 | −0.1944 | 0.7669 | 2.1823 | 1.2209 | 2 | 1 |
| WCM14-TP1 | 1 | 193104708 | 208062471 | 0.2764 | 1.3701 | 3.067 | 2.1027 | 3 | 2 |
| WCM14-TP1 | 1 | 208062973 | 249212577 | −0.1768 | 0.7906 | 2.2279 | 1.2316 | 2 | 1 |
| WCM14-TP1 | 10 | 386619 | 45938452 | 0.1351 | 1.1946 | 2.539 | 2.0386 | 3 | 2 |
| WCM14-TP1 | 10 | 45984878 | 70516035 | −0.33 | 0.5814 | 1.8213 | 1.1712 | 2 | 1 |
| WCM14-TP1 | 10 | 70517064 | 81113586 | 0.5454 | 1.6937 | 3.9765 | 2.4934 | 4 | 2 |
| WCM14-TP1 | 10 | 81145992 | 135053877 | −0.495 | 0.346 | 2.3489 | 0.1932 | 2 | 0 |
| WCM14-TP1 | 10 | 135061128 | 135379000 | −0.0389 | 0.9724 | 2.6963 | 1.2279 | 3 | 1 |
| WCM14-TP1 | 11 | 193096 | 1026393 | 0.154 | 1.2183 | 3.7634 | 0.8901 | 4 | 1 |
| WCM14-TP1 | 11 | 1027089 | 4410925 | 0.095 | 1.144 | 3.5247 | 0.8952 | 4 | 1 |
| WCM14-TP1 | 11 | 4411334 | 6942748 | −0.7634 | −0.0655 | 1.7872 | 0.124 | 2 | 0 |
| WCM14-TP1 | 11 | 6943258 | 47350681 | −0.1479 | 0.8291 | 2.9821 | 0.571 | 3 | 1 |
| WCM14-TP1 | 11 | 47350876 | 134257753 | −0.0293 | 0.9848 | 2.7567 | 1.2015 | 3 | 1 |
| WCM14-TP1 | 12 | 247582 | 4854809 | 0.0597 | 1.0992 | 2.4181 | 1.8667 | 2 | 2 |
| WCM14-TP1 | 12 | 4855365 | 38712138 | −0.3061 | 0.6146 | 2.0861 | 0.9761 | 2 | 1 |
| WCM14-TP1 | 12 | 38712265 | 92538251 | 0.0075 | 1.0323 | 2.1901 | 1.9005 | 2 | 2 |
| WCM14-TP1 | 12 | 92539243 | 133810751 | 0.5683 | 1.7208 | 4.1194 | 2.4729 | 4 | 2 |
| WCM14-TP1 | 13 | 19601063 | 45841502 | −0.6186 | 0.1616 | 2.0499 | 0.1871 | 2 | 0 |
| WCM14-TP1 | 13 | 45857737 | 111099057 | −1.164 | −0.7821 | 1.0864 | 0.0766 | 1 | 0 |
| WCM14-TP1 | 13 | 111102042 | 115089411 | −0.3529 | 0.5493 | 2.7 | 0.2268 | 3 | 0 |
| WCM14-TP1 | 14 | 19110188 | 20296498 | 1.0002 | 2.2177 | 7.8365 | 1.4663 | 8 | 1 |
| WCM14-TP1 | 14 | 20344415 | 100119096 | 0.1216 | 1.1776 | 2.4957 | 2.0284 | 2 | 2 |
| WCM14-TP1 | 14 | 100123516 | 107283269 | 0.4812 | 1.6176 | 3.383 | 2.7542 | 3 | 3 |
| WCM14-TP1 | 15 | 20087605 | 25287234 | −0.4946 | 0.3467 | 1.7252 | 0.8181 | 2 | 1 |
| WCM14-TP1 | 15 | 25523590 | 102389659 | −0.53 | 0.2946 | 1.4914 | 0.9617 | 1 | 1 |
| WCM14-TP1 | 16 | 97605 | 29912794 | 0.1316 | 1.1902 | 2.6118 | 1.9519 | 3 | 2 |
| WCM14-TP1 | 16 | 29912970 | 80584342 | −0.367 | 0.5295 | 1.7501 | 1.1368 | 2 | 1 |
| WCM14-TP1 | 16 | 80638455 | 81697861 | −0.9929 | −0.4567 | 1.2248 | 0.2325 | 1 | 0 |
| WCM14-TP1 | 16 | 81703861 | 85697023 | −0.4428 | 0.4218 | 1.6383 | 1.0409 | 2 | 1 |
| WCM14-TP1 | 16 | 85697189 | 90168697 | 0.0309 | 1.0625 | 2.6397 | 1.5373 | 3 | 2 |
| WCM14-TP1 | 17 | 131595 | 5462581 | 0.2155 | 1.2949 | 2.8246 | 2.0826 | 3 | 2 |
| WCM14-TP1 | 17 | 5463034 | 21207909 | −0.5753 | 0.2271 | 2.1425 | 0.1984 | 2 | 0 |
| WCM14-TP1 | 17 | 21208317 | 81052332 | 0.1413 | 1.2024 | 2.6276 | 1.9748 | 3 | 2 |
| WCM14-TP1 | 18 | 47669 | 21116858 | −0.0181 | 0.9994 | 2.5915 | 1.4069 | 3 | 1 |
| WCM14-TP1 | 18 | 21118575 | 78005164 | −0.6538 | 0.1075 | 2.0129 | 0.1418 | 2 | 0 |
| WCM14-TP1 | 19 | 281543 | 7998998 | −0.2632 | 0.6736 | 2.9481 | 0.242 | 3 | 0 |
| WCM14-TP1 | 19 | 8000001 | 8987271 | 0.0071 | 1.0319 | 2.6079 | 1.4814 | 3 | 1 |
| WCM14-TP1 | 19 | 8993027 | 9033814 | 1.7257 | 3.0134 | 8.9929 | 7.1566 | 9 | 7 |
| WCM14-TP1 | 19 | 9038114 | 11687709 | 0.0965 | 1.146 | 2.4622 | 1.9638 | 2 | 2 |
| WCM14-TP1 | 19 | 12761041 | 19774034 | 0.1011 | 1.1518 | 2.6433 | 1.8005 | 3 | 2 |
| WCM14-TP1 | 19 | 19779736 | 30498329 | 1.3536 | 2.6102 | 6.1728 | 6.0389 | 6 | 6 |
| WCM14-TP1 | 19 | 30499976 | 52794204 | 0.1101 | 1.1631 | 2.5333 | 1.9454 | 3 | 2 |
| WCM14-TP1 | 19 | 52794430 | 54307360 | 1.6865 | 2.9714 | 7.9103 | 7.7754 | 8 | 8 |
| WCM14-TP1 | 19 | 54308554 | 59063088 | 0.1743 | 1.2437 | 2.7021 | 2.0342 | 3 | 2 |
| WCM14-TP1 | 2 | 41463 | 84851763 | −0.196 | 0.7647 | 2.1952 | 1.2029 | 2 | 1 |
| WCM14-TP1 | 2 | 84852135 | 179363945 | −0.1459 | 0.8318 | 2.0261 | 1.5337 | 2 | 2 |
| WCM14-TP1 | 2 | 179365928 | 179646981 | −0.8849 | −0.2672 | 0.8522 | 0.8097 | 1 | 1 |
| WCM14-TP1 | 2 | 179647305 | 238462324 | −0.2235 | 0.7276 | 1.8421 | 1.4697 | 2 | 1 |
| WCM14-TP1 | 2 | 238475353 | 242946604 | −0.0271 | 0.9877 | 2.4996 | 1.4664 | 2 | 1 |
| WCM14-TP1 | 20 | 68299 | 30053478 | 0.1258 | 1.183 | 2.5734 | 1.9675 | 3 | 2 |
| WCM14-TP1 | 20 | 30060755 | 56186831 | −0.3452 | 0.5601 | 1.8785 | 1.0703 | 2 | 1 |
| WCM14-TP1 | 20 | 56188273 | 62905001 | 0.0754 | 1.1192 | 2.9476 | 1.3968 | 3 | 1 |
| WCM14-TP1 | 21 | 9907274 | 14414929 | 0.6919 | 1.8653 | 5.2722 | 2.0146 | 5 | 2 |
| WCM14-TP1 | 21 | 14418567 | 17138408 | −1.1175 | −0.6902 | 1.385 | −0.1455 | 1 | 0 |
| WCM14-TP1 | 21 | 17150327 | 48084142 | 0.1642 | 1.2311 | 2.6291 | 2.0657 | 3 | 2 |
| WCM14-TP1 | 22 | 17052984 | 51219090 | −0.1749 | 0.793 | 3.2407 | 0.2247 | 3 | 0 |
| WCM14-TP1 | 3 | 239390 | 7678056 | 0.313 | 1.4149 | 3.1057 | 2.2272 | 3 | 2 |
| WCM14-TP1 | 3 | 7721890 | 16216844 | −0.5079 | 0.3271 | 2.4314 | 0.0776 | 2 | 0 |
| WCM14-TP1 | 3 | 16217040 | 33093489 | 0.3685 | 1.4822 | 2.8817 | 2.7059 | 3 | 3 |
| WCM14-TP1 | 3 | 33095121 | 37369939 | −0.4611 | 0.3954 | 1.77 | 0.8606 | 2 | 1 |
| WCM14-TP1 | 3 | 37370562 | 45583332 | 0.4014 | 1.522 | 3.1971 | 2.5468 | 3 | 3 |
| WCM14-TP1 | 3 | 45588967 | 53925849 | −0.5043 | 0.3325 | 2.3116 | 0.2067 | 2 | 0 |
| WCM14-TP1 | 3 | 54157554 | 59997166 | 0.6386 | 1.8033 | 3.535 | 3.4453 | 4 | 3 |
| WCM14-TP1 | 3 | 59999668 | 89528659 | −0.3188 | 0.5969 | 2.9929 | 0.0321 | 3 | 0 |
| WCM14-TP1 | 3 | 90255162 | 113440586 | 0.0064 | 1.031 | 2.0697 | 2.0171 | 2 | 2 |
| WCM14-TP1 | 3 | 125701243 | 182631792 | 0.2151 | 1.2945 | 2.6201 | 2.2857 | 3 | 2 |
| WCM14-TP1 | 3 | 182631831 | 184144822 | −0.1771 | 0.7902 | 2.2297 | 1.2289 | 2 | 1 |
| WCM14-TP1 | 3 | 184157036 | 197847453 | 0.2064 | 1.2837 | 2.4587 | 2.4104 | 2 | 2 |
| WCM14-TP1 | 4 | 53446 | 48581017 | −0.1604 | 0.8124 | 2.253 | 1.2593 | 2 | 1 |
| WCM14-TP1 | 4 | 48581297 | 72631387 | 0.3462 | 1.4552 | 2.7653 | 2.7187 | 3 | 3 |
| WCM14-TP1 | 4 | 74010459 | 85648096 | 0.0563 | 1.0949 | 2.8086 | 1.4633 | 3 | 1 |
| WCM14-TP1 | 4 | 85654497 | 141330994 | −0.3676 | 0.5287 | 1.875 | 1.0102 | 2 | 1 |
| WCM14-TP1 | 4 | 141331746 | 187203041 | −0.0404 | 0.9704 | 2.079 | 1.8397 | 2 | 2 |
| WCM14-TP1 | 5 | 140696 | 50074439 | 0.3313 | 1.4372 | 2.8595 | 2.5563 | 3 | 3 |
| WCM14-TP1 | 5 | 50084313 | 180687828 | −0.2777 | 0.6537 | 2.999 | 0.1473 | 3 | 0 |
| WCM14-TP1 | 6 | 203552 | 62687960 | −0.2763 | 0.6556 | 2.0338 | 1.1168 | 2 | 1 |
| WCM14-TP1 | 6 | 69685114 | 109980643 | −0.2584 | 0.6801 | 2.0336 | 1.1709 | 2 | 1 |
| WCM14-TP1 | 6 | 109982120 | 134303661 | −0.0354 | 0.9769 | 2.7451 | 1.1914 | 3 | 1 |
| WCM14-TP1 | 6 | 134303921 | 149285680 | −0.3466 | 0.5582 | 1.6825 | 1.2623 | 2 | 1 |
| WCM14-TP1 | 6 | 149340390 | 152675990 | −1.1431 | −0.7404 | 1.0788 | 0.1183 | 1 | 0 |
| WCM14-TP1 | 6 | 152679647 | 160219189 | −0.4253 | 0.4468 | 1.9072 | 0.8189 | 2 | 1 |
| WCM14-TP1 | 6 | 160221442 | 161162406 | 0.7195 | 1.8973 | 4.0357 | 3.4147 | 4 | 3 |
| WCM14-TP1 | 6 | 161173313 | 170892853 | −0.3963 | 0.4881 | 2.1847 | 0.6206 | 2 | 1 |
| WCM14-TP1 | 7 | 195579 | 6661628 | 0.1071 | 1.1594 | 2.8825 | 1.5848 | 3 | 2 |
| WCM14-TP1 | 7 | 6662314 | 36463419 | −0.2895 | 0.6374 | 1.8791 | 1.2319 | 2 | 1 |
| WCM14-TP1 | 7 | 36464274 | 158937375 | 0.2351 | 1.3192 | 2.9485 | 2.0422 | 3 | 2 |
| WCM14-TP1 | 8 | 190850 | 43027457 | −0.574 | 0.2291 | 2.1626 | 0.1816 | 2 | 0 |
| WCM14-TP1 | 8 | 43028897 | 146279489 | 0.8697 | 2.0698 | 6.0553 | 2.3415 | 6 | 2 |
| WCM14-TP1 | 9 | 214535 | 17502798 | −1.1212 | −0.6974 | 1.1452 | 0.0881 | 1 | 0 |
| WCM14-TP1 | 9 | 17747173 | 21968256 | 0.518 | 1.6614 | 6.2086 | 0.1178 | 6 | 0 |
| WCM14-TP1 | 9 | 21968695 | 32784695 | −0.2596 | 0.6785 | 3.0618 | 0.139 | 3 | 0 |
| WCM14-TP1 | 9 | 32785006 | 69830308 | 0.0536 | 1.0915 | 2.3892 | 1.8727 | 2 | 2 |
| WCM14-TP1 | 9 | 71144551 | 141016359 | −0.1153 | 0.8723 | 3.4577 | 0.2033 | 3 | 0 |
| WCM14-TP1 | X | 2700166 | 65835817 | 0.7727 | 1.9588 | NA | NA | NA | NA |
| WCM14-TP1 | X | 66765138 | 68060476 | 2.6185 | 3.9539 | NA | NA | NA | NA |
| WCM14-TP1 | X | 68381246 | 92965154 | −0.643 | 0.1242 | NA | NA | NA | NA |
| WCM14-TP1 | X | 94318093 | 154275641 | 0.1216 | 1.1777 | NA | NA | NA | NA |
| WCM14-TP1 | X | 154282988 | 154509348 | −0.7932 | −0.1139 | NA | NA | NA | NA |
| WCM14-TP2 | 1 | 762244 | 108307731 | −0.0902 | 0.837 | 2.3135 | 1.2591 | 2 | 1 |
| WCM14-TP2 | 1 | 108309202 | 111773600 | −0.5303 | 0.1715 | 1.9589 | 0.2936 | 2 | 0 |
| WCM14-TP2 | 1 | 111773882 | 197480029 | −0.1288 | 0.7824 | 2.1306 | 1.3094 | 2 | 1 |
| WCM14-TP2 | 1 | 197481015 | 207958406 | 0.3417 | 1.4127 | 3.0552 | 2.2696 | 3 | 2 |
| WCM14-TP2 | 1 | 207959070 | 249212589 | −0.1645 | 0.7315 | 2.0609 | 1.2597 | 2 | 1 |
| WCM14-TP2 | 10 | 226083 | 45938452 | 0.1675 | 1.1873 | 2.3342 | 2.2202 | 2 | 2 |
| WCM14-TP2 | 10 | 45984844 | 70502209 | −0.2999 | 0.5327 | 1.7441 | 1.1493 | 2 | 1 |
| WCM14-TP2 | 10 | 70502336 | 81343131 | 0.5978 | 1.7313 | 4.0118 | 2.6286 | 4 | 3 |
| WCM14-TP2 | 10 | 81372081 | 135101582 | −0.4862 | 0.2434 | 2.2045 | 0.163 | 2 | 0 |
| WCM14-TP2 | 10 | 135101839 | 135378985 | −0.0581 | 0.8819 | 3.3034 | 0.3822 | 3 | 0 |
| WCM14-TP2 | 11 | 193096 | 4159719 | 0.0211 | 0.991 | 3.4248 | 0.5504 | 3 | 1 |
| WCM14-TP2 | 11 | 4185612 | 7067950 | −0.7247 | −0.1633 | 1.8325 | −0.0466 | 2 | 0 |
| WCM14-TP2 | 11 | 7068117 | 43852491 | −0.119 | 0.7963 | 3.0531 | 0.4201 | 3 | 0 |
| WCM14-TP2 | 11 | 43859820 | 134257738 | 0.0201 | 0.9897 | 2.8231 | 1.1484 | 3 | 1 |
| WCM14-TP2 | 12 | 247582 | 313816 | 0.8612 | 2.0463 | NA | NA | NA | NA |
| WCM14-TP2 | 12 | 319111 | 4854828 | 0.1147 | 1.1173 | 2.2099 | 2.1289 | 2 | 2 |
| WCM14-TP2 | 12 | 4855365 | 28122893 | −0.2362 | 0.6273 | 2.1074 | 0.982 | 2 | 1 |
| WCM14-TP2 | 12 | 28410146 | 49496723 | −0.0123 | 0.9453 | 1.9785 | 1.8725 | 2 | 2 |
| WCM14-TP2 | 12 | 49496978 | 53443061 | 0.3525 | 1.4265 | 2.7186 | 2.6572 | 3 | 3 |
| WCM14-TP2 | 12 | 53443982 | 91502602 | 0.0025 | 0.9656 | 1.9689 | 1.9368 | 2 | 2 |
| WCM14-TP2 | 12 | 91502721 | 133810751 | 0.6377 | 1.7798 | 4.307 | 2.5607 | 4 | 3 |
| WCM14-TP2 | 13 | 19601109 | 45915151 | −0.5798 | 0.0893 | 1.9937 | 0.1341 | 2 | 0 |
| WCM14-TP2 | 13 | 45970090 | 110408608 | −1.1189 | −0.9817 | 0.9814 | 0.0314 | 1 | 0 |
| WCM14-TP2 | 13 | 110434474 | 115089411 | −0.3301 | 0.4873 | 2.4435 | 0.3601 | 2 | 0 |
| WCM14-TP2 | 14 | 19110188 | 107283269 | 0.178 | 1.2011 | 2.4564 | 2.142 | 2 | 2 |
| WCM14-TP2 | 15 | 20087605 | 25287198 | −0.4054 | 0.3714 | 1.8183 | 0.769 | 2 | 1 |
| WCM14-TP2 | 15 | 25296754 | 25495991 | 1.8014 | 3.1028 | NA | NA | NA | NA |
| WCM14-TP2 | 15 | 25514925 | 102389685 | −0.529 | 0.1737 | 1.2798 | 0.976 | 1 | 1 |
| WCM14-TP2 | 16 | 97423 | 33961553 | 0.1246 | 1.1305 | 2.4283 | 1.9505 | 2 | 2 |
| WCM14-TP2 | 16 | 33965589 | 79632669 | −0.4244 | 0.3416 | 1.4364 | 1.0979 | 1 | 1 |
| WCM14-TP2 | 16 | 79632894 | 82197704 | −0.9422 | −0.5849 | 1.0686 | 0.2647 | 1 | 0 |
| WCM14-TP2 | 16 | 82203705 | 89735933 | −0.2522 | 0.6038 | 1.7598 | 1.2796 | 2 | 1 |
| WCM14-TP2 | 16 | 89753225 | 90168687 | −0.0978 | 0.8263 | 1.913 | 1.6333 | 2 | 2 |
| WCM14-TP2 | 17 | 131595 | 5462349 | 0.2042 | 1.2355 | 2.407 | 2.3023 | 2 | 2 |
| WCM14-TP2 | 17 | 5462627 | 21201719 | −0.5638 | 0.1161 | 2.0927 | 0.075 | 2 | 0 |
| WCM14-TP2 | 17 | 21201851 | 81052303 | 0.1656 | 1.1848 | 2.519 | 2.0275 | 3 | 2 |
| WCM14-TP2 | 18 | 47668 | 19444692 | 0.0053 | 0.9694 | 2.4086 | 1.5074 | 2 | 2 |
| WCM14-TP2 | 18 | 19751212 | 78005215 | −0.565 | 0.1141 | 2.0595 | 0.105 | 2 | 0 |
| WCM14-TP2 | 19 | 281405 | 7990225 | −0.3554 | 0.4486 | 2.5283 | 0.2012 | 3 | 0 |
| WCM14-TP2 | 19 | 7992052 | 8982235 | 0.305 | 1.3659 | 2.6665 | 2.4882 | 3 | 2 |
| WCM14-TP2 | 19 | 8987127 | 9038066 | 1.8683 | 3.1752 | 9.5503 | 8.516 | 10 | 9 |
| WCM14-TP2 | 19 | 9038331 | 11687654 | 0.0736 | 1.0623 | 2.1051 | 2.0714 | 2 | 2 |
| WCM14-TP2 | 19 | 11688130 | 12576173 | 1.4268 | 2.6917 | NA | NA | NA | NA |
| WCM14-TP2 | 19 | 12576453 | 19843548 | 0.1484 | 1.1621 | 2.2586 | 2.2171 | 2 | 2 |
| WCM14-TP2 | 19 | 19843810 | 30020678 | 1.5574 | 2.8362 | 7.3066 | 6.9762 | 7 | 7 |
| WCM14-TP2 | 19 | 30021023 | 52793878 | 0.1783 | 1.2015 | 2.3482 | 2.2514 | 2 | 2 |
| WCM14-TP2 | 19 | 52794291 | 54304483 | 1.785 | 3.085 | 8.4855 | 8.4855 | 8 | 8 |
| WCM14-TP2 | 19 | 54307249 | 59083951 | 0.225 | 1.2626 | 2.4336 | 2.3649 | 2 | 2 |
| WCM14-TP2 | 2 | 41612 | 86357774 | −0.1457 | 0.7583 | 2.1988 | 1.1842 | 2 | 1 |
| WCM14-TP2 | 2 | 86358285 | 179367086 | −0.1335 | 0.7758 | 1.9948 | 1.4295 | 2 | 1 |
| WCM14-TP2 | 2 | 179391834 | 179647077 | −0.7374 | −0.1864 | 1.0182 | 0.7393 | 1 | 1 |
| WCM14-TP2 | 2 | 179647314 | 242590329 | −0.1554 | 0.7445 | 1.8937 | 1.457 | 2 | 1 |
| WCM14-TP2 | 2 | 242590775 | 242946657 | −0.1567 | 0.7426 | 1.8027 | 1.5437 | 2 | 2 |
| WCM14-TP2 | 20 | 68375 | 25448025 | 0.1369 | 1.1468 | 2.2348 | 2.1936 | 2 | 2 |
| WCM14-TP2 | 20 | 25450742 | 30897493 | −0.124 | 0.7892 | 2.5031 | 0.9532 | 3 | 1 |
| WCM14-TP2 | 20 | 30897883 | 41101170 | −0.4352 | 0.3247 | 1.2959 | 1.2089 | 1 | 1 |
| WCM14-TP2 | 20 | 41306600 | 56275268 | −0.2379 | 0.6248 | 1.9748 | 1.1091 | 2 | 1 |
| WCM14-TP2 | 20 | 56284586 | 62905001 | 0.0822 | 1.0738 | 2.7388 | 1.4713 | 3 | 1 |
| WCM14-TP2 | 21 | 9907274 | 14414913 | 0.4233 | 1.5158 | 3.9527 | 1.7666 | 4 | 2 |
| WCM14-TP2 | 21 | 14418567 | 16340289 | −0.8351 | −0.3699 | 1.5543 | −0.0066 | 2 | 0 |
| WCM14-TP2 | 21 | 16340454 | 48084142 | 0.1983 | 1.2278 | 2.3635 | 2.3206 | 2 | 2 |
| WCM14-TP2 | 22 | 17052984 | 51219089 | −0.1556 | 0.7443 | 3.1579 | 0.1924 | 3 | 0 |
| WCM14-TP2 | 3 | 239524 | 7678056 | 0.3205 | 1.3858 | 2.7199 | 2.5063 | 3 | 3 |
| WCM14-TP2 | 3 | 7721826 | 16216760 | −0.4749 | 0.2615 | 2.2159 | 0.1815 | 2 | 0 |
| WCM14-TP2 | 3 | 16217079 | 33093505 | 0.3197 | 1.3848 | 2.6913 | 2.5312 | 3 | 3 |
| WCM14-TP2 | 3 | 33095121 | 37369294 | −0.3555 | 0.4485 | 1.6863 | 1.0429 | 2 | 1 |
| WCM14-TP2 | 3 | 37369984 | 45714194 | 0.3385 | 1.4087 | 2.8644 | 2.4454 | 3 | 2 |
| WCM14-TP2 | 3 | 45715065 | 49701108 | −0.7041 | −0.1261 | 1.83 | 0.0026 | 2 | 0 |
| WCM14-TP2 | 3 | 49701298 | 52722099 | −0.3127 | 0.5134 | 1.5264 | 1.3285 | 2 | 1 |
| WCM14-TP2 | 3 | 52723268 | 53925849 | −0.6576 | −0.044 | 1.8408 | 0.0992 | 2 | 0 |
| WCM14-TP2 | 3 | 54157497 | 61911540 | 0.5926 | 1.725 | 3.3903 | 3.2211 | 3 | 3 |
| WCM14-TP2 | 3 | 61975520 | 90264953 | −0.1864 | 0.6999 | 3.2308 | 0.018 | 3 | 0 |
| WCM14-TP2 | 3 | 90274768 | 113392205 | 0.0796 | 1.0703 | 2.1083 | 2.0914 | 2 | 2 |
| WCM14-TP2 | 3 | 113393740 | 125694511 | −0.5425 | 0.1515 | NA | NA | NA | NA |
| WCM14-TP2 | 3 | 125701277 | 183696402 | 0.2347 | 1.2753 | 2.4424 | 2.3984 | 2 | 2 |
| WCM14-TP2 | 3 | 183699421 | 184003328 | −0.3488 | 0.4587 | 1.6849 | 1.0638 | 2 | 1 |
| WCM14-TP2 | 3 | 184005619 | 197847416 | 0.1987 | 1.2283 | 2.3614 | 2.3246 | 2 | 2 |
| WCM14-TP2 | 4 | 53446 | 55593685 | −0.1581 | 0.7406 | 2.1785 | 1.1631 | 2 | 1 |
| WCM14-TP2 | 4 | 55594073 | 72622601 | 0.4247 | 1.5175 | 2.8809 | 2.845 | 3 | 3 |
| WCM14-TP2 | 4 | 72623766 | 74008518 | −0.3021 | 0.5295 | NA | NA | NA | NA |
| WCM14-TP2 | 4 | 74010439 | 85648072 | 0.1647 | 1.1836 | 2.8766 | 1.6664 | 3 | 2 |
| WCM14-TP2 | 4 | 85654688 | 141560576 | −0.3089 | 0.5193 | 1.7935 | 1.073 | 2 | 1 |
| WCM14-TP2 | 4 | 141563742 | 187188343 | 0.0284 | 1.001 | 2.0096 | 1.9932 | 2 | 2 |
| WCM14-TP2 | 4 | 187192933 | 190862042 | −0.6666 | −0.0597 | NA | NA | NA | NA |
| WCM14-TP2 | 5 | 140603 | 44388817 | 0.3756 | 1.4558 | 2.7777 | 2.7084 | 3 | 3 |
| WCM14-TP2 | 5 | 44809120 | 180687847 | −0.2249 | 0.6439 | 2.9621 | 0.1631 | 3 | 0 |
| WCM14-TP2 | 6 | 203546 | 62442604 | −0.1825 | 0.7056 | 2.0217 | 1.2399 | 2 | 1 |
| WCM14-TP2 | 6 | 62604611 | 69348586 | −1.3488 | −1.621 | NA | NA | NA | NA |
| WCM14-TP2 | 6 | 69348859 | 148852745 | −0.1547 | 0.7454 | 2.1854 | 1.1675 | 2 | 1 |
| WCM14-TP2 | 6 | 148854058 | 152660451 | −1.0228 | −0.7585 | 1.1212 | 0.0611 | 1 | 0 |
| WCM14-TP2 | 6 | 152665261 | 159686065 | −0.3939 | 0.3892 | 1.871 | 0.7484 | 2 | 1 |
| WCM14-TP2 | 6 | 159687149 | 166580358 | 0.2077 | 1.24 | 2.7447 | 1.9794 | 3 | 2 |
| WCM14-TP2 | 6 | 166581034 | 170892873 | −0.51 | 0.2047 | 2.1245 | 0.1804 | 2 | 0 |
| WCM14-TP2 | 7 | 193346 | 1518526 | −0.0067 | 0.953 | NA | NA | NA | NA |
| WCM14-TP2 | 7 | 1519056 | 38259291 | −0.1342 | 0.7747 | 2.1175 | 1.3042 | 2 | 1 |
| WCM14-TP2 | 7 | 38268395 | 38785245 | 0.279 | 1.3325 | 2.7292 | 2.3077 | 3 | 2 |
| WCM14-TP2 | 7 | 38791843 | 39874024 | 1.5625 | 2.8418 | NA | NA | NA | NA |
| WCM14-TP2 | 7 | 39990283 | 56183977 | 0.1036 | 1.1024 | 2.5061 | 1.7882 | 3 | 2 |
| WCM14-TP2 | 7 | 56358862 | 65552703 | 1.5709 | 2.8511 | 7.7078 | 6.7233 | 8 | 7 |
| WCM14-TP2 | 7 | 65553833 | 158937357 | 0.2374 | 1.2787 | 2.7507 | 2.1017 | 3 | 2 |
| WCM14-TP2 | 8 | 190912 | 43046564 | −0.5288 | 0.174 | 2.1191 | 0.1372 | 2 | 0 |
| WCM14-TP2 | 8 | 43047497 | 146279479 | 0.9353 | 2.1329 | 6.2974 | 2.4749 | 6 | 2 |
| WCM14-TP2 | 9 | 214597 | 17502798 | −0.999 | −0.706 | 1.1292 | 0.0969 | 1 | 0 |
| WCM14-TP2 | 9 | 17579296 | 20981684 | 0.7714 | 1.9402 | 7.5052 | 0.17 | 8 | 0 |
| WCM14-TP2 | 9 | 20982416 | 32541894 | −0.1723 | 0.7202 | 3.2103 | 0.0846 | 3 | 0 |
| WCM14-TP2 | 9 | 32542204 | 71225850 | −7.00E−04 | 0.9612 | 2.2808 | 1.6131 | 2 | 2 |
| WCM14-TP2 | 9 | 71395145 | 141016459 | −0.0745 | 0.859 | 3.4532 | 0.1743 | 3 | 0 |
| WCM14-TP2 | X | 2700108 | 65835822 | 0.8077 | 1.9832 | NA | NA | NA | NA |
| WCM14-TP2 | X | 66765033 | 68060461 | 2.5993 | 3.9514 | NA | NA | NA | NA |
| WCM14-TP2 | X | 68381264 | 96140046 | −0.5702 | 0.1055 | NA | NA | NA | NA |
| WCM14-TP2 | X | 96167471 | 154509348 | 0.1475 | 1.1608 | NA | NA | NA | NA |
| WCM14-TP3 | 1 | 762244 | 109177786 | −0.0938 | 0.8271 | 2.4313 | 1.1169 | 2 | 1 |
| WCM14-TP3 | 1 | 109191376 | 111785067 | −0.4183 | −0.0788 | 1.7866 | 0.1071 | 2 | 0 |
| WCM14-TP3 | 1 | 111824318 | 152185500 | −0.1378 | 0.7231 | 2.1647 | 1.1368 | 2 | 1 |
| WCM14-TP3 | 1 | 152185767 | 152328479 | 1.2979 | 3.0779 | 9.349 | 7.5385 | 9 | 8 |
| WCM14-TP3 | 1 | 152328908 | 201020105 | −0.0992 | 0.8145 | 2.2703 | 1.2472 | 2 | 1 |
| WCM14-TP3 | 1 | 201021692 | 206651179 | 0.3299 | 1.6673 | 3.2422 | 3.11 | 3 | 3 |
| WCM14-TP3 | 1 | 206651624 | 249212760 | −0.1254 | 0.7527 | 2.3279 | 1.0421 | 2 | 1 |
| WCM14-TP3 | 10 | 225982 | 70514563 | −0.003 | 1.0292 | 2.4127 | 1.6691 | 2 | 2 |
| WCM14-TP3 | 10 | 70515093 | 81113488 | 0.4899 | 1.9345 | 4.6781 | 2.9668 | 5 | 3 |
| WCM14-TP3 | 10 | 81145992 | 135279810 | −0.3057 | 0.2786 | 2.3478 | 0.0782 | 2 | 0 |
| WCM14-TP3 | 10 | 135280765 | 135378971 | 0.2469 | 1.5201 | NA | NA | NA | NA |
| WCM14-TP3 | 11 | 193096 | 1031238 | −0.0168 | 0.9995 | 3.3944 | 0.6042 | 3 | 1 |
| WCM14-TP3 | 11 | 1031814 | 4408265 | 0.1521 | 1.3433 | 4.1836 | 0.8911 | 4 | 1 |
| WCM14-TP3 | 11 | 4409527 | 7065086 | −0.7423 | −1.8014 | 1.2695 | −0.6957 | 1 | −1 |
| WCM14-TP3 | 11 | 7067929 | 43852491 | −0.0876 | 0.8414 | 3.1118 | 0.4717 | 3 | 0 |
| WCM14-TP3 | 11 | 43859976 | 134257753 | −0.0484 | 0.9301 | 2.732 | 1.0789 | 3 | 1 |
| WCM14-TP3 | 12 | 247582 | 4543574 | 0.1144 | 1.27 | 2.5397 | 2.2835 | 3 | 2 |
| WCM14-TP3 | 12 | 4553391 | 40631959 | −0.0967 | 0.8203 | 2.438 | 1.0935 | 2 | 1 |
| WCM14-TP3 | 12 | 40634388 | 92538265 | 0.0209 | 1.0799 | 2.1361 | 2.0916 | 2 | 2 |
| WCM14-TP3 | 12 | 92539254 | 133770121 | 0.4861 | 1.9284 | 4.6352 | 2.9775 | 5 | 3 |
| WCM14-TP3 | 12 | 133778796 | 133810935 | −0.4409 | −0.1588 | NA | NA | NA | NA |
| WCM14-TP3 | 13 | 19601153 | 45841502 | −0.3725 | 0.074 | 2.1158 | −0.0106 | 2 | 0 |
| WCM14-TP3 | 13 | 45857595 | 110895092 | −0.7717 | −2.0951 | 0.6962 | −0.2281 | 1 | 0 |
| WCM14-TP3 | 13 | 110959368 | 115089411 | −0.1113 | 0.7862 | 3.1489 | 0.3002 | 3 | 0 |
| WCM14-TP3 | 14 | 19110188 | 107283270 | 0.1095 | 1.2605 | 2.6417 | 2.1498 | 3 | 2 |
| WCM14-TP3 | 15 | 20087605 | 25287205 | −0.1645 | 0.6577 | 2.4223 | 0.7328 | 2 | 1 |
| WCM14-TP3 | 15 | 25296735 | 25515021 | 2.3115 | 4.2798 | NA | NA | NA | NA |
| WCM14-TP3 | 15 | 25523593 | 102389747 | −0.355 | 0.1294 | 1.3441 | 0.8437 | 1 | 1 |
| WCM14-TP3 | 16 | 97515 | 28916320 | 0.1456 | 1.3309 | 2.7871 | 2.244 | 3 | 2 |
| WCM14-TP3 | 16 | 28916759 | 85696657 | −0.2769 | 0.3611 | 1.6896 | 0.8792 | 2 | 1 |
| WCM14-TP3 | 16 | 85697100 | 90168701 | 0.0803 | 1.2023 | 2.681 | 1.921 | 3 | 2 |
| WCM14-TP3 | 17 | 131595 | 5433341 | 0.1327 | 1.3058 | 2.5539 | 2.3905 | 3 | 2 |
| WCM14-TP3 | 17 | 5433941 | 21730703 | −0.4493 | −0.1893 | 1.9044 | −0.1503 | 2 | 0 |
| WCM14-TP3 | 17 | 21825435 | 81052303 | 0.1205 | 1.2821 | 2.6941 | 2.1699 | 3 | 2 |
| WCM14-TP3 | 18 | 47661 | 20573029 | 0.0395 | 1.1189 | 2.6989 | 1.6447 | 3 | 2 |
| WCM14-TP3 | 18 | 20573217 | 74680114 | −0.4045 | −0.0316 | 1.9817 | −0.0251 | 2 | 0 |
| WCM14-TP3 | 18 | 74692281 | 78005150 | −0.2208 | 0.514 | NA | NA | NA | NA |
| WCM14-TP3 | 19 | 281435 | 7992625 | −0.2376 | 0.4693 | 2.5741 | 0.1948 | 3 | 0 |
| WCM14-TP3 | 19 | 7992959 | 8987187 | 0.1838 | 1.4036 | 2.7303 | 2.561 | 3 | 3 |
| WCM14-TP3 | 19 | 8993008 | 9027274 | 1.9636 | 3.8839 | 16.6952 | 12.8313 | 17 | 13 |
| WCM14-TP3 | 19 | 9027623 | 11688156 | 0.0589 | 1.1588 | 2.2783 | 2.187 | 2 | 2 |
| WCM14-TP3 | 19 | 11708464 | 12576269 | 1.1582 | 2.8971 | NA | NA | NA | NA |
| WCM14-TP3 | 19 | 12576371 | 19843634 | 0.037 | 1.1136 | 2.2089 | 2.1189 | 2 | 2 |
| WCM14-TP3 | 19 | 19905342 | 30020678 | 1.4624 | 3.2848 | 10.0598 | 9.4317 | 10 | 9 |
| WCM14-TP3 | 19 | 30020992 | 52794022 | 0.0493 | 1.139 | 2.4517 | 1.9528 | 2 | 2 |
| WCM14-TP3 | 19 | 52794243 | 54301537 | 1.4193 | 3.2312 | 9.484 | 9.2964 | 9 | 9 |
| WCM14-TP3 | 19 | 54304572 | 59083923 | 0.1188 | 1.2788 | 2.5692 | 2.2836 | 3 | 2 |
| WCM14-TP3 | 2 | 41602 | 179341953 | −0.1156 | 0.776 | 2.1574 | 1.2674 | 2 | 1 |
| WCM14-TP3 | 2 | 179343217 | 186678786 | −0.6198 | −0.9457 | 0.5534 | 0.4849 | 1 | 0 |
| WCM14-TP3 | 2 | 186680141 | 189874873 | −0.0493 | 0.9281 | 1.9748 | 1.8307 | 2 | 2 |
| WCM14-TP3 | 2 | 189875382 | 234098481 | −0.0998 | 0.8131 | 1.7939 | 1.7202 | 2 | 2 |
| WCM14-TP3 | 2 | 234102624 | 242946531 | −0.0258 | 0.9798 | 2.4803 | 1.4642 | 2 | 1 |
| WCM14-TP3 | 20 | 68351 | 3218127 | −0.1542 | 0.6833 | 1.783 | 1.4285 | 2 | 1 |
| WCM14-TP3 | 20 | 3218634 | 25448091 | 0.146 | 1.3317 | 2.5606 | 2.4733 | 3 | 2 |
| WCM14-TP3 | 20 | 25450682 | 62905001 | −0.1528 | 0.6865 | 2.0667 | 1.152 | 2 | 1 |
| WCM14-TP3 | 21 | 9907274 | 48084196 | 0.1343 | 1.309 | 2.5257 | 2.4295 | 3 | 2 |
| WCM14-TP3 | 22 | 17052984 | 51219090 | −0.1119 | 0.7849 | 3.3617 | 0.0842 | 3 | 0 |
| WCM14-TP3 | 3 | 239495 | 7728097 | 0.1329 | 1.3062 | 2.9102 | 2.0355 | 3 | 2 |
| WCM14-TP3 | 3 | 7732961 | 16216961 | −0.26 | 0.4083 | 2.5717 | 0.0826 | 3 | 0 |
| WCM14-TP3 | 3 | 16217145 | 45714211 | 0.2184 | 1.4681 | 2.9246 | 2.6084 | 3 | 3 |
| WCM14-TP3 | 3 | 45714902 | 49040173 | −0.3822 | 0.0425 | 2.1936 | −0.1338 | 2 | 0 |
| WCM14-TP3 | 3 | 49042344 | 49720779 | −1.0777 | NA | 0.4039 | −0.917 | 0 | −1 |
| WCM14-TP3 | 3 | 49728896 | 53925849 | −0.272 | 0.375 | 2.5347 | 0.0589 | 3 | 0 |
| WCM14-TP3 | 3 | 54157639 | 61975493 | 0.4941 | 1.9413 | 3.9639 | 3.7169 | 4 | 4 |
| WCM14-TP3 | 3 | 61989192 | 75378434 | −0.192 | 0.5885 | 3.0577 | −0.0503 | 3 | 0 |
| WCM14-TP3 | 3 | 75378546 | 113376954 | 0.0363 | 1.1122 | 2.2112 | 2.1123 | 2 | 2 |
| WCM14-TP3 | 3 | 113377361 | 125271511 | −0.412 | −0.0571 | 1.5245 | 0.3978 | 2 | 0 |
| WCM14-TP3 | 3 | 125272968 | 197847416 | 0.1299 | 1.3003 | 2.7267 | 2.1991 | 3 | 2 |
| WCM14-TP3 | 4 | 53446 | 55133936 | −0.1267 | 0.7498 | 2.1473 | 1.2159 | 2 | 1 |
| WCM14-TP3 | 4 | 55136853 | 72897868 | 0.2997 | 1.6145 | 3.1214 | 3.0027 | 3 | 3 |
| WCM14-TP3 | 4 | 72934632 | 190862042 | −0.1258 | 0.752 | 2.0256 | 1.3426 | 2 | 1 |
| WCM14-TP3 | 5 | 140637 | 44388956 | 0.2971 | 1.6099 | 3.0847 | 3.02 | 3 | 3 |
| WCM14-TP3 | 5 | 44809186 | 140731357 | −0.1776 | 0.6251 | 3.1081 | −0.0235 | 3 | 0 |
| WCM14-TP3 | 5 | 140731451 | 141337225 | −0.6936 | −1.4075 | 1.7457 | −0.9917 | 2 | −1 |
| WCM14-TP3 | 5 | 141353338 | 180687821 | −0.1852 | 0.6058 | 3.0832 | −0.0395 | 3 | 0 |
| WCM14-TP3 | 6 | 203428 | 150464488 | −0.178 | 0.6241 | 2.0643 | 1.0181 | 2 | 1 |
| WCM14-TP3 | 6 | 150535957 | 152675994 | −0.9304 | NA | 0.4405 | −0.5071 | 0 | −1 |
| WCM14-TP3 | 6 | 152679571 | 170892853 | −0.122 | 0.761 | 2.464 | 0.9253 | 2 | 1 |
| WCM14-TP3 | 7 | 193359 | 4780430 | 0.1474 | 1.3343 | 3.1721 | 1.871 | 3 | 2 |
| WCM14-TP3 | 7 | 4780658 | 36571999 | −0.0927 | 0.8296 | 2.2581 | 1.2963 | 2 | 1 |
| WCM14-TP3 | 7 | 36580045 | 56183977 | 0.1706 | 1.3787 | 3.1529 | 2.0478 | 3 | 2 |
| WCM14-TP3 | 7 | 56358862 | 64441754 | 1.6636 | 3.5302 | 12.1779 | 10.9281 | 12 | 11 |
| WCM14-TP3 | 7 | 64451558 | 158937361 | 0.2185 | 1.4681 | 3.3327 | 2.2005 | 3 | 2 |
| WCM14-TP3 | 8 | 190907 | 43027457 | −0.3798 | 0.0501 | 2.1167 | −0.046 | 2 | 0 |
| WCM14-TP3 | 8 | 43028806 | 144650332 | 0.7634 | 2.3526 | 7.3618 | 2.8528 | 7 | 3 |
| WCM14-TP3 | 8 | 144651821 | 145750114 | 0.0908 | 1.2232 | 4.328 | 0.3415 | 4 | 0 |
| WCM14-TP3 | 8 | 145753014 | 146279529 | 0.6529 | 2.1886 | NA | NA | NA | NA |
| WCM14-TP3 | 9 | 214494 | 17579270 | −0.6452 | −1.0911 | 0.9907 | −0.0519 | 1 | 0 |
| WCM14-TP3 | 9 | 17747173 | 21968293 | 0.4914 | 1.9369 | 7.1997 | 0.4582 | 7 | 0 |
| WCM14-TP3 | 9 | 21968678 | 33473846 | −0.1921 | 0.5884 | 2.881 | 0.1261 | 3 | 0 |
| WCM14-TP3 | 9 | 33528813 | 34088551 | 0.5946 | 2.0996 | NA | NA | NA | NA |
| WCM14-TP3 | 9 | 34089395 | 74562066 | −0.1874 | 0.6002 | 1.6122 | 1.4197 | 2 | 1 |
| WCM14-TP3 | 9 | 74586468 | 141016451 | −0.0317 | 0.9671 | 3.8117 | 0.0981 | 4 | 0 |
| WCM14-TP3 | X | 2700159 | 65835841 | 0.6025 | 2.1117 | NA | NA | NA | NA |
| WCM14-TP3 | X | 66765039 | 68060470 | 2.2709 | 4.2344 | NA | NA | NA | NA |
| WCM14-TP3 | X | 68381222 | 99663472 | −0.4952 | −0.365 | NA | NA | NA | NA |
| WCM14-TP3 | X | 99840019 | 154275612 | 0.1065 | 1.2544 | NA | NA | NA | NA |
| WCM14-TP3 | X | 154282994 | 154509348 | −0.4333 | −0.1315 | NA | NA | NA | NA |
| WCM14-TP4 | 1 | 762244 | 108309127 | −0.1206 | 0.7541 | 2.2201 | 1.1531 | 2 | 1 |
| WCM14-TP4 | 1 | 108311173 | 111217122 | −0.6955 | −0.1035 | 1.8091 | 0.0524 | 2 | 0 |
| WCM14-TP4 | 1 | 111217233 | 193099346 | −0.155 | 0.707 | 2.1533 | 1.1115 | 2 | 1 |
| WCM14-TP4 | 1 | 193104708 | 207245580 | 0.3538 | 1.3684 | 2.7799 | 2.3838 | 3 | 2 |
| WCM14-TP4 | 1 | 207252295 | 249212725 | −0.1302 | 0.741 | 2.2538 | 1.0889 | 2 | 1 |
| WCM14-TP4 | 10 | 225938 | 49450424 | 0.1025 | 1.0505 | 2.1169 | 2.0256 | 2 | 2 |
| WCM14-TP4 | 10 | 49452867 | 70531217 | −0.2358 | 0.5947 | 1.9347 | 1.0855 | 2 | 1 |
| WCM14-TP4 | 10 | 70531237 | 70666793 | 0.2949 | 1.2952 | NA | NA | NA | NA |
| WCM14-TP4 | 10 | 70670104 | 81343103 | 0.6873 | 1.7709 | 4.238 | 2.5875 | 4 | 3 |
| WCM14-TP4 | 10 | 81372081 | 135378940 | −0.5202 | 0.1767 | 2.1937 | 0.067 | 2 | 0 |
| WCM14-TP4 | 11 | 193096 | 4562903 | 0.0257 | 0.9501 | 3.3775 | 0.4865 | 3 | 0 |
| WCM14-TP4 | 11 | 4566572 | 7065231 | −0.7894 | −0.2626 | 1.7377 | −0.0705 | 2 | 0 |
| WCM14-TP4 | 11 | 7067923 | 11964668 | −0.1523 | 0.7107 | 2.8784 | 0.3949 | 3 | 0 |
| WCM14-TP4 | 11 | 11969414 | 21392487 | −0.0147 | 0.8967 | 3.4183 | 0.3052 | 3 | 0 |
| WCM14-TP4 | 11 | 21556002 | 48328606 | −0.1167 | 0.7594 | 2.8756 | 0.51 | 3 | 1 |
| WCM14-TP4 | 11 | 48346648 | 134257766 | 0.0358 | 0.9633 | 2.7007 | 1.1989 | 3 | 1 |
| WCM14-TP4 | 12 | 247582 | 4830082 | 0.1686 | 1.1356 | 2.2111 | 2.183 | 2 | 2 |
| WCM14-TP4 | 12 | 4835755 | 38712161 | −0.2266 | 0.6075 | 2.1689 | 0.8784 | 2 | 1 |
| WCM14-TP4 | 12 | 38712265 | 51636326 | 0.1157 | 1.0675 | 2.1093 | 2.0823 | 2 | 2 |
| WCM14-TP4 | 12 | 51639763 | 53442983 | 0.5455 | 1.6021 | 3.0468 | 3.0249 | 3 | 3 |
| WCM14-TP4 | 12 | 53443938 | 92538269 | −0.0442 | 0.8574 | 1.8214 | 1.802 | 2 | 2 |
| WCM14-TP4 | 12 | 92539254 | 133778796 | 0.6526 | 1.7299 | 4.2288 | 2.4053 | 4 | 2 |
| WCM14-TP4 | 12 | 133779108 | 133810735 | −0.123 | 0.7509 | NA | NA | NA | NA |
| WCM14-TP4 | 13 | 19601153 | 45841502 | −0.6222 | 0.0161 | 1.9532 | 0.0692 | 2 | 0 |
| WCM14-TP4 | 13 | 45857742 | 111102128 | −1.1943 | −1.0601 | 0.9232 | 0.036 | 1 | 0 |
| WCM14-TP4 | 13 | 111102728 | 115089411 | −0.419 | 0.33 | 2.3453 | 0.1687 | 2 | 0 |
| WCM14-TP4 | 14 | 19110188 | 107283269 | 0.2181 | 1.1985 | 2.4812 | 2.109 | 2 | 2 |
| WCM14-TP4 | 15 | 20087605 | 25287234 | −0.4032 | 0.3533 | 1.6991 | 0.856 | 2 | 1 |
| WCM14-TP4 | 15 | 25296719 | 25515016 | 1.5225 | 2.7158 | NA | NA | NA | NA |
| WCM14-TP4 | 15 | 25523597 | 102389685 | −0.5678 | 0.1025 | 1.1327 | 1.0146 | 1 | 1 |
| WCM14-TP4 | 16 | 97503 | 34681533 | 0.1754 | 1.1442 | 2.2357 | 2.1849 | 2 | 2 |
| WCM14-TP4 | 16 | 34681928 | 73127412 | −0.4709 | 0.252 | 1.2937 | 1.0881 | 1 | 1 |
| WCM14-TP4 | 16 | 73161291 | 77246795 | 0.0271 | 0.9519 | 2.4006 | 1.4683 | 2 | 1 |
| WCM14-TP4 | 16 | 77317756 | 80577227 | −0.1932 | 0.6542 | 1.6088 | 1.5388 | 2 | 2 |
| WCM14-TP4 | 16 | 80581631 | 81735431 | −0.9684 | −0.5891 | 1.2576 | 0.0719 | 1 | 0 |
| WCM14-TP4 | 16 | 81736324 | 90110950 | −0.246 | 0.5803 | 1.6469 | 1.3434 | 2 | 1 |
| WCM14-TP4 | 16 | 90124787 | 90168697 | 0.0102 | 0.9296 | NA | NA | NA | NA |
| WCM14-TP4 | 17 | 131595 | 5461799 | 0.2513 | 1.2405 | 2.3926 | 2.3331 | 2 | 2 |
| WCM14-TP4 | 17 | 5461970 | 21207909 | −0.623 | 0.0149 | 1.9941 | 0.0267 | 2 | 0 |
| WCM14-TP4 | 17 | 21208449 | 81052321 | 0.1834 | 1.1544 | 2.2373 | 2.2146 | 2 | 2 |
| WCM14-TP4 | 18 | 47669 | 19444692 | 0.0295 | 0.9551 | 2.3775 | 1.4998 | 2 | 1 |
| WCM14-TP4 | 18 | 19751215 | 78005164 | −0.6036 | 0.046 | 2.013 | 0.0518 | 2 | 0 |
| WCM14-TP4 | 19 | 281404 | 7990208 | −0.4153 | 0.3355 | 2.4054 | 0.1182 | 2 | 0 |
| WCM14-TP4 | 19 | 7992052 | 8977614 | 0.2701 | 1.2642 | 2.438 | 2.3657 | 2 | 2 |
| WCM14-TP4 | 19 | 8979199 | 9033680 | 2.0385 | 3.2717 | 10.3457 | 8.9694 | 10 | 9 |
| WCM14-TP4 | 19 | 9038116 | 11687888 | 0.0918 | 1.0365 | 2.0566 | 2.046 | 2 | 2 |
| WCM14-TP4 | 19 | 11688178 | 12576371 | 1.2073 | 2.3672 | NA | NA | NA | NA |
| WCM14-TP4 | 19 | 12577245 | 19824936 | 0.2112 | 1.1898 | 2.2929 | 2.2697 | 2 | 2 |
| WCM14-TP4 | 19 | 19825314 | 30020678 | 1.5422 | 2.7372 | 7.345 | 5.9908 | 7 | 6 |
| WCM14-TP4 | 19 | 30021023 | 52794214 | 0.1402 | 1.0991 | 2.2386 | 2.0458 | 2 | 2 |
| WCM14-TP4 | 19 | 52794435 | 54304573 | 1.3147 | 2.487 | 5.6184 | 5.5935 | 6 | 6 |
| WCM14-TP4 | 19 | 54307335 | 59083953 | 0.1056 | 1.0544 | 2.0957 | 2.0581 | 2 | 2 |
| WCM14-TP4 | 2 | 41595 | 173913468 | −0.1735 | 0.6815 | 1.9629 | 1.2447 | 2 | 1 |
| WCM14-TP4 | 2 | 173916507 | 186678794 | −0.596 | 0.0581 | 1.056 | 1.0262 | 1 | 1 |
| WCM14-TP4 | 2 | 186680222 | 242946607 | −0.1351 | 0.7343 | 1.8753 | 1.4518 | 2 | 1 |
| WCM14-TP4 | 20 | 68413 | 377219 | −0.8348 | −0.3423 | NA | NA | NA | NA |
| WCM14-TP4 | 20 | 377341 | 25507117 | 0.1383 | 1.0966 | 2.1606 | 2.1165 | 2 | 2 |
| WCM14-TP4 | 20 | 25596639 | 30454862 | −0.0679 | 0.8255 | 2.7786 | 0.7656 | 3 | 1 |
| WCM14-TP4 | 20 | 30457341 | 40710558 | −0.4567 | 0.2735 | 1.3071 | 1.1104 | 1 | 1 |
| WCM14-TP4 | 20 | 40713478 | 62905001 | −0.0802 | 0.809 | 2.2084 | 1.2956 | 2 | 1 |
| WCM14-TP4 | 21 | 9907274 | 14414885 | 0.214 | 1.1934 | NA | NA | NA | NA |
| WCM14-TP4 | 21 | 14418567 | 17163811 | −0.9039 | −0.4675 | 1.4834 | −0.037 | 1 | 0 |
| WCM14-TP4 | 21 | 17172133 | 48084211 | 0.2387 | 1.2246 | 2.3487 | 2.3251 | 2 | 2 |
| WCM14-TP4 | 22 | 17052984 | 51219090 | −0.2283 | 0.6052 | 2.9424 | 0.0999 | 3 | 0 |
| WCM14-TP4 | 3 | 239390 | 7678056 | 0.3573 | 1.3727 | 3.1853 | 1.9939 | 3 | 2 |
| WCM14-TP4 | 3 | 7721887 | 16216718 | −0.4916 | 0.2206 | 2.2498 | 0.0805 | 2 | 0 |
| WCM14-TP4 | 3 | 16217091 | 33093505 | 0.3765 | 1.3965 | 2.7657 | 2.4995 | 3 | 2 |
| WCM14-TP4 | 3 | 33095121 | 37369237 | −0.3615 | 0.4146 | 1.835 | 0.8309 | 2 | 1 |
| WCM14-TP4 | 3 | 37370061 | 45588862 | 0.2972 | 1.2981 | 2.4806 | 2.4376 | 2 | 2 |
| WCM14-TP4 | 3 | 45636355 | 49701148 | −0.7299 | −0.1609 | 1.832 | −0.0431 | 2 | 0 |
| WCM14-TP4 | 3 | 49701298 | 52864584 | −0.3796 | 0.3882 | 1.8853 | 0.7323 | 2 | 1 |
| WCM14-TP4 | 3 | 52867288 | 53925849 | −0.7462 | −0.1884 | 1.8225 | −0.0674 | 2 | 0 |
| WCM14-TP4 | 3 | 54157662 | 61975367 | 0.6618 | 1.7408 | 3.366 | 3.3185 | 3 | 3 |
| WCM14-TP4 | 3 | 61989192 | 85985017 | −0.1333 | 0.7368 | 3.2917 | 0.0413 | 3 | 0 |
| WCM14-TP4 | 3 | 86010600 | 113392192 | 0.0722 | 1.0111 | 2.0364 | 1.9944 | 2 | 2 |
| WCM14-TP4 | 3 | 113393747 | 125691052 | −0.6212 | 0.0178 | NA | NA | NA | NA |
| WCM14-TP4 | 3 | 125694554 | 182631792 | 0.247 | 1.235 | 2.3806 | 2.3272 | 2 | 2 |
| WCM14-TP4 | 3 | 182631843 | 184070076 | −0.2454 | 0.5812 | 1.9242 | 1.0679 | 2 | 1 |
| WCM14-TP4 | 3 | 184070332 | 197847390 | 0.2142 | 1.1936 | 2.3017 | 2.2727 | 2 | 2 |
| WCM14-TP4 | 4 | 53446 | 55156400 | −0.2117 | 0.6285 | 1.8957 | 1.1962 | 2 | 1 |
| WCM14-TP4 | 4 | 55156739 | 72634975 | 0.4015 | 1.4273 | 2.7026 | 2.6762 | 3 | 3 |
| WCM14-TP4 | 4 | 72649819 | 74088854 | −0.342 | 0.443 | NA | NA | NA | NA |
| WCM14-TP4 | 4 | 74124154 | 85648113 | 0.0895 | 1.0336 | 2.5978 | 1.4965 | 3 | 1 |
| WCM14-TP4 | 4 | 85654548 | 142642271 | −0.3368 | 0.4506 | 1.6893 | 1.0439 | 2 | 1 |
| WCM14-TP4 | 4 | 142643062 | 187179210 | 1.00E−04 | 0.9163 | 1.8998 | 1.8748 | 2 | 2 |
| WCM14-TP4 | 4 | 187188406 | 190862042 | −0.5393 | 0.1472 | NA | NA | NA | NA |
| WCM14-TP4 | 5 | 140712 | 44388822 | 0.3669 | 1.3847 | 2.6208 | 2.6014 | 3 | 3 |
| WCM14-TP4 | 5 | 44809079 | 139893044 | −0.2335 | 0.5979 | 2.9297 | 0.0973 | 3 | 0 |
| WCM14-TP4 | 5 | 139903824 | 141359805 | −0.7203 | −0.1448 | 2.0213 | −0.2123 | 2 | 0 |
| WCM14-TP4 | 5 | 141362974 | 180687847 | −0.1889 | 0.6602 | 3.0673 | 0.0933 | 3 | 0 |
| WCM14-TP4 | 6 | 203479 | 62687896 | −0.2077 | 0.634 | 1.9588 | 1.1449 | 2 | 1 |
| WCM14-TP4 | 6 | 62757908 | 66803535 | −1.4967 | −1.873 | NA | NA | NA | NA |
| WCM14-TP4 | 6 | 66803647 | 110036274 | −0.2439 | 0.5832 | 1.8613 | 1.1351 | 2 | 1 |
| WCM14-TP4 | 6 | 110037614 | 133767925 | −0.0042 | 0.9107 | 2.6701 | 1.0897 | 3 | 1 |
| WCM14-TP4 | 6 | 133769200 | 149285623 | −0.2891 | 0.5192 | 1.7892 | 1.0772 | 2 | 1 |
| WCM14-TP4 | 6 | 149340390 | 152688397 | −1.1754 | −1.0175 | 0.9755 | 0.0125 | 1 | 0 |
| WCM14-TP4 | 6 | 152690092 | 158850920 | −0.1461 | 0.7192 | 2.132 | 1.1606 | 2 | 1 |
| WCM14-TP4 | 6 | 158870063 | 159672491 | −0.9283 | −0.5129 | 1.4194 | −0.0178 | 1 | 0 |
| WCM14-TP4 | 6 | 159677528 | 160209172 | −0.1396 | 0.7282 | NA | NA | NA | NA |
| WCM14-TP4 | 6 | 160210407 | 166579270 | 0.2073 | 1.1848 | 2.7698 | 1.7769 | 3 | 2 |
| WCM14-TP4 | 6 | 166580193 | 170892825 | −0.6023 | 0.0481 | 1.9627 | 0.1051 | 2 | 0 |
| WCM14-TP4 | 7 | 193346 | 822145 | 0.2021 | 1.1783 | NA | NA | NA | NA |
| WCM14-TP4 | 7 | 822522 | 38254631 | −0.09 | 0.7957 | 2.1635 | 1.3085 | 2 | 1 |
| WCM14-TP4 | 7 | 38256597 | 38768322 | 0.3331 | 1.3428 | NA | NA | NA | NA |
| WCM14-TP4 | 7 | 38780883 | 40132331 | 1.4162 | 2.5991 | 10.0223 | 2.0959 | 10 | 2 |
| WCM14-TP4 | 7 | 40132599 | 56155396 | 0.2135 | 1.1927 | 2.3496 | 2.222 | 2 | 2 |
| WCM14-TP4 | 7 | 56155519 | 65447001 | 1.4584 | 2.6455 | 6.4632 | 6.051 | 6 | 6 |
| WCM14-TP4 | 7 | 65541029 | 158937361 | 0.3511 | 1.3651 | 2.8853 | 2.2667 | 3 | 2 |
| WCM14-TP4 | 8 | 190879 | 43054665 | −0.5656 | 0.1061 | 2.1067 | 0.0459 | 2 | 0 |
| WCM14-TP4 | 8 | 43147666 | 144650419 | 1.0349 | 2.173 | 6.5686 | 2.4503 | 7 | 2 |
| WCM14-TP4 | 8 | 144651838 | 146279349 | 0.5238 | 1.576 | 4.678 | 1.2849 | 5 | 1 |
| WCM14-TP4 | 9 | 214656 | 17579306 | −1.0884 | −0.8293 | 1.0707 | 0.0549 | 1 | 0 |
| WCM14-TP4 | 9 | 17747173 | 21968164 | 0.6846 | 1.7678 | 6.6037 | 0.2068 | 7 | 0 |
| WCM14-TP4 | 9 | 21968695 | 32541876 | −0.2355 | 0.5952 | 3.0969 | −0.0756 | 3 | 0 |
| WCM14-TP4 | 9 | 32542204 | 71225850 | −0.0453 | 0.8559 | 1.965 | 1.6548 | 2 | 2 |
| WCM14-TP4 | 9 | 71395044 | 141016459 | −0.0808 | 0.8081 | 3.3653 | 0.1364 | 3 | 0 |
| WCM14-TP4 | X | 2700084 | 65238850 | 0.7669 | 1.8643 | NA | NA | NA | NA |
| WCM14-TP4 | X | 65241997 | 68060461 | 2.2546 | 3.5005 | NA | NA | NA | NA |
| WCM14-TP4 | X | 68381282 | 96140065 | −0.7377 | −0.1741 | NA | NA | NA | NA |
| WCM14-TP4 | X | 96167496 | 153735196 | 0.17 | 1.1374 | NA | NA | NA | NA |
| WCM14-TP4 | X | 153760604 | 153940861 | −0.5194 | 0.1779 | NA | NA | NA | NA |
| WCM14-TP4 | X | 153941673 | 154509348 | 0.3368 | 1.3473 | NA | NA | NA | NA |
| WCM14_x1_1 | 1 | 762178 | 145096653 | −0.1975 | 0.6499 | 2.0503 | 1.0877 | 2 | 1 |
| WCM14_x1_1 | 1 | 145103928 | 249212761 | 0.0216 | 1.0443 | 2.0798 | 2.0448 | 2 | 2 |
| WCM14_x1_1 | 10 | 225995 | 38120558 | 0.01 | 1.0245 | 2.0516 | 2.0168 | 2 | 2 |
| WCM14_x1_1 | 10 | 38121054 | 70229735 | 0.1307 | 1.2249 | 2.3752 | 2.2997 | 2 | 2 |
| WCM14_x1_1 | 10 | 70229941 | 70502336 | 0.0408 | 1.0767 | NA | NA | NA | NA |
| WCM14_x1_1 | 10 | 70507061 | 77818559 | 0.4741 | 1.7451 | 4.1572 | 2.5473 | 4 | 3 |
| WCM14_x1_1 | 10 | 78084125 | 81317809 | 0.9943 | 2.4416 | NA | NA | NA | NA |
| WCM14_x1_1 | 10 | 81318621 | 112582191 | −0.3426 | 0.3574 | 2.6494 | −0.0871 | 3 | 0 |
| WCM14_x1_1 | 10 | 112583342 | 135379059 | −0.4792 | 0.0503 | 2.0575 | 0.0135 | 2 | 0 |
| WCM14_x1_1 | 11 | 193096 | 15994502 | 0.0049 | 1.0159 | 2.0429 | 2.0013 | 2 | 2 |
| WCM14_x1_1 | 11 | 16007889 | 51527499 | −0.2243 | 0.598 | 1.9978 | 1.0294 | 2 | 1 |
| WCM14_x1_1 | 11 | 55110668 | 134257807 | 0.1947 | 1.3268 | 2.9783 | 2.0388 | 3 | 2 |
| WCM14_x1_1 | 12 | 247582 | 39069551 | −0.1978 | 0.6493 | 2.0796 | 1.0571 | 2 | 1 |
| WCM14_x1_1 | 12 | 39071220 | 89919715 | 0.236 | 1.3914 | 3.0254 | 2.2211 | 3 | 2 |
| WCM14_x1_1 | 12 | 89984716 | 133810874 | 0.0109 | 1.026 | 2.0572 | 2.0155 | 2 | 2 |
| WCM14_x1_1 | 13 | 19601049 | 115089411 | −0.4766 | 0.0564 | 2.0711 | 0.0087 | 2 | 0 |
| WCM14_x1_1 | 14 | 19110203 | 107283263 | 0.0183 | 1.0387 | 2.0648 | 2.0438 | 2 | 2 |
| WCM14_x1_1 | 15 | 20087605 | 22440726 | −0.1645 | 0.7125 | 2.7681 | 0.5091 | 3 | 1 |
| WCM14_x1_1 | 15 | 22448436 | 37387827 | −0.1878 | 0.6685 | 1.9407 | 1.2381 | 2 | 1 |
| WCM14_x1_1 | 15 | 37388647 | 102389672 | 0.0074 | 1.02 | 2.0453 | 2.0106 | 2 | 2 |
| WCM14_x1_1 | 16 | 97424 | 34682250 | 0.0514 | 1.0945 | 2.1568 | 2.1139 | 2 | 2 |
| WCM14_x1_1 | 16 | 46508189 | 72993860 | −0.1477 | 0.7438 | 2.1914 | 1.1577 | 2 | 1 |
| WCM14_x1_1 | 16 | 72993919 | 76461500 | 0.0707 | 1.1267 | 2.2923 | 2.075 | 2 | 2 |
| WCM14_x1_1 | 16 | 76481940 | 80583456 | −0.1745 | 0.6936 | 1.8863 | 1.3484 | 2 | 1 |
| WCM14_x1_1 | 16 | 80584342 | 82660739 | −0.7766 | −0.7996 | 1.09 | 0.059 | 1 | 0 |
| WCM14_x1_1 | 16 | 82892037 | 90168701 | −0.1544 | 0.7313 | 2.0383 | 1.2821 | 2 | 1 |
| WCM14_x1_1 | 17 | 131595 | 5463332 | 0.0071 | 1.0195 | 2.0619 | 1.9925 | 2 | 2 |
| WCM14_x1_1 | 17 | 5485295 | 21188211 | −0.4859 | 0.034 | 2.0411 | 0.0067 | 2 | 0 |
| WCM14_x1_1 | 17 | 21201719 | 81052366 | 0.026 | 1.0518 | 2.0941 | 2.052 | 2 | 2 |
| WCM14_x1_1 | 18 | 47665 | 19444692 | −0.0431 | 0.9327 | 1.949 | 1.8687 | 2 | 2 |
| WCM14_x1_1 | 18 | 19751194 | 78005243 | −0.4954 | 0.0113 | 2.0356 | −0.0198 | 2 | 0 |
| WCM14_x1_1 | 19 | 281445 | 7853194 | −0.4755 | 0.059 | 2.1457 | −0.0622 | 2 | 0 |
| WCM14_x1_1 | 19 | 7911577 | 59083968 | 0.0227 | 1.0461 | 2.0649 | 2.0649 | 2 | 2 |
| WCM14_x1_1 | 2 | 41595 | 242946597 | −0.0189 | 0.9748 | 1.9857 | 1.9449 | 2 | 2 |
| WCM14_x1_1 | 20 | 68375 | 36149831 | 0.0364 | 1.0694 | 2.1233 | 2.0738 | 2 | 2 |
| WCM14_x1_1 | 20 | 36150779 | 37662857 | −0.2052 | 0.635 | 2.0706 | 1.0354 | 2 | 1 |
| WCM14_x1_1 | 20 | 37667223 | 45140783 | 0.0375 | 1.0711 | 2.2391 | 1.963 | 2 | 2 |
| WCM14_x1_1 | 20 | 45169921 | 62905001 | −0.1826 | 0.6783 | 2.0986 | 1.1018 | 2 | 1 |
| WCM14_x1_1 | 21 | 9826166 | 17116365 | −0.4937 | 0.0154 | 2.0126 | 0.0088 | 2 | 0 |
| WCM14_x1_1 | 21 | 17135323 | 48084196 | 0.0054 | 1.0166 | 2.0474 | 1.9989 | 2 | 2 |
| WCM14_x1_1 | 22 | 16255826 | 51219089 | −0.045 | 0.9293 | 1.9277 | 1.8809 | 2 | 2 |
| WCM14_x1_1 | 3 | 239405 | 7678010 | 0.3716 | 1.5963 | 3.8047 | 2.2428 | 4 | 2 |
| WCM14_x1_1 | 3 | 7721765 | 15915357 | −0.3659 | 0.3074 | 2.5731 | −0.0981 | 3 | 0 |
| WCM14_x1_1 | 3 | 16216733 | 33058287 | 0.3997 | 1.6375 | 3.6164 | 2.6063 | 4 | 3 |
| WCM14_x1_1 | 3 | 33059967 | 33728618 | −0.398 | 0.2371 | NA | NA | NA | NA |
| WCM14_x1_1 | 3 | 33729756 | 41953086 | 0.0579 | 1.1054 | 2.2664 | 2.0366 | 2 | 2 |
| WCM14_x1_1 | 3 | 41954265 | 45636356 | 0.4276 | 1.6782 | 3.7708 | 2.6296 | 4 | 3 |
| WCM14_x1_1 | 3 | 45636831 | 49751551 | −0.3836 | 0.269 | 2.4185 | −0.0086 | 2 | 0 |
| WCM14_x1_1 | 3 | 49753144 | 52726769 | 0.1455 | 1.2486 | NA | NA | NA | NA |
| WCM14_x1_1 | 3 | 52726992 | 54157609 | −0.4861 | 0.0336 | 2.0135 | 0.0336 | 2 | 0 |
| WCM14_x1_1 | 3 | 54354431 | 59997092 | 0.4851 | 1.7608 | 3.4901 | 3.2874 | 3 | 3 |
| WCM14_x1_1 | 3 | 59999866 | 90274787 | −0.1953 | 0.654 | 3.1701 | −0.0231 | 3 | 0 |
| WCM14_x1_1 | 3 | 90306533 | 113464797 | 0.274 | 1.4498 | 2.9695 | 2.494 | 3 | 2 |
| WCM14_x1_1 | 3 | 113497697 | 125400268 | −0.3866 | 0.2625 | NA | NA | NA | NA |
| WCM14_x1_1 | 3 | 125690929 | 128363758 | 0.4147 | 1.6595 | 3.8764 | 2.4418 | 4 | 2 |
| WCM14_x1_1 | 3 | 128369437 | 142840524 | 0.246 | 1.4068 | 2.7047 | 2.5984 | 3 | 3 |
| WCM14_x1_1 | 3 | 142840735 | 147106695 | 0.3455 | 1.5577 | 3.9036 | 1.9839 | 4 | 2 |
| WCM14_x1_1 | 3 | 147108697 | 170244659 | 0.4131 | 1.6571 | 3.9393 | 2.3685 | 4 | 2 |
| WCM14_x1_1 | 3 | 170425809 | 180359790 | 0.2472 | 1.4087 | 2.7532 | 2.5567 | 3 | 3 |
| WCM14_x1_1 | 3 | 180361950 | 184035229 | 0.1193 | 1.2064 | 2.36 | 2.2551 | 2 | 2 |
| WCM14_x1_1 | 3 | 184035306 | 197847416 | 0.3028 | 1.4936 | 3.1605 | 2.4712 | 3 | 2 |
| WCM14_x1_1 | 4 | 53446 | 48896047 | −0.2018 | 0.6415 | 2.0518 | 1.0681 | 2 | 1 |
| WCM14_x1_1 | 4 | 48899857 | 83424202 | 0.0193 | 1.0404 | 2.0848 | 2.0289 | 2 | 2 |
| WCM14_x1_1 | 4 | 83425726 | 151770138 | −0.107 | 0.8186 | 2.0551 | 1.4722 | 2 | 1 |
| WCM14_x1_1 | 4 | 151770561 | 190862042 | 0.0233 | 1.0471 | 2.1049 | 2.0278 | 2 | 2 |
| WCM14_x1_1 | 5 | 140685 | 45696169 | 0.02 | 1.0415 | 2.0898 | 2.0269 | 2 | 2 |
| WCM14_x1_1 | 5 | 49695023 | 68800075 | −0.8654 | −1.1383 | 0.8954 | 0.0131 | 1 | 0 |
| WCM14_x1_1 | 5 | 68804987 | 180687854 | −0.1966 | 0.6516 | 3.136 | 0.0057 | 3 | 0 |
| WCM14_x1_1 | 6 | 203374 | 397277 | −0.0776 | 0.8716 | NA | NA | NA | NA |
| WCM14_x1_1 | 6 | 398888 | 57512718 | −0.1263 | 0.7835 | 2.328 | 1.1145 | 2 | 1 |
| WCM14_x1_1 | 6 | 62284247 | 69653778 | −0.4508 | 0.1172 | 2.1572 | 0.0121 | 2 | 0 |
| WCM14_x1_1 | 6 | 69665899 | 85473891 | −0.0573 | 0.9077 | 1.9125 | 1.8395 | 2 | 2 |
| WCM14_x1_1 | 6 | 86160065 | 89675638 | 0.2896 | 1.4735 | NA | NA | NA | NA |
| WCM14_x1_1 | 6 | 89790810 | 90097350 | −0.5673 | −0.17 | NA | NA | NA | NA |
| WCM14_x1_1 | 6 | 90121630 | 110620349 | 0.1222 | 1.2111 | 2.7619 | 1.8684 | 3 | 2 |
| WCM14_x1_1 | 6 | 110636524 | 135263579 | 0.3 | 1.4894 | 3.7759 | 1.8394 | 4 | 2 |
| WCM14_x1_1 | 6 | 135264946 | 149068790 | 0.1199 | 1.2073 | 2.8376 | 1.7806 | 3 | 2 |
| WCM14_x1_1 | 6 | 149208148 | 152673420 | −0.6044 | −0.2692 | 1.701 | −0.0414 | 2 | 0 |
| WCM14_x1_1 | 6 | 152674455 | 158735342 | 0.106 | 1.1848 | 2.96 | 1.5865 | 3 | 2 |
| WCM14_x1_1 | 6 | 158834198 | 159692405 | −0.5538 | −0.1348 | 1.6557 | 0.1659 | 2 | 0 |
| WCM14_x1_1 | 6 | 160103568 | 166581100 | 0.1501 | 1.2561 | 3.1348 | 1.6421 | 3 | 2 |
| WCM14_x1_1 | 6 | 166720806 | 170892860 | −0.6062 | −0.2742 | 1.6358 | 0.018 | 2 | 0 |
| WCM14_x1_1 | 7 | 193355 | 56126419 | 0.0041 | 1.0144 | 2.0477 | 1.9923 | 2 | 2 |
| WCM14_x1_1 | 7 | 56128090 | 158937357 | 0.4163 | 1.6618 | 4.1585 | 2.1699 | 4 | 2 |
| WCM14_x1_1 | 8 | 190897 | 48068823 | −0.4816 | 0.0444 | 2.0564 | 0.0061 | 2 | 0 |
| WCM14_x1_1 | 8 | 48114127 | 146279324 | 0.9367 | 2.3683 | 8.2334 | 2.0932 | 8 | 2 |
| WCM14_x1_1 | 9 | 214650 | 17747173 | −0.8 | −0.8833 | 1.1286 | −0.0443 | 1 | 0 |
| WCM14_x1_1 | 9 | 17761444 | 20821092 | 0.2586 | 1.4262 | 5.3529 | 0.0217 | 5 | 0 |
| WCM14_x1_1 | 9 | 20823142 | 32635545 | −0.2703 | 0.5068 | 2.8194 | 0.0224 | 3 | 0 |
| WCM14_x1_1 | 9 | 32784246 | 34297030 | 0.6745 | 2.0237 | 4.2811 | 3.8516 | 4 | 4 |
| WCM14_x1_1 | 9 | 34306436 | 66513346 | 0.393 | 1.6277 | 3.9463 | 2.2342 | 4 | 2 |
| WCM14_x1_1 | 9 | 66514076 | 141016459 | −0.4559 | 0.1052 | 2.1821 | −0.0308 | 2 | 0 |
| WCM14_x1_1 | X | 2700146 | 17092203 | 0.2093 | 1.3498 | NA | NA | NA | NA |
| WCM14_x1_1 | X | 17095487 | 65835787 | 0.2717 | 1.4464 | NA | NA | NA | NA |
| WCM14_x1_1 | X | 66765033 | 67751827 | 1.1607 | 2.649 | NA | NA | NA | NA |
| WCM14_x1_1 | X | 67867798 | 82764141 | 0.2888 | 1.4723 | NA | NA | NA | NA |
| WCM14_x1_1 | X | 82764391 | 154509348 | 0.1566 | 1.2665 | NA | NA | NA | NA |
| WCM14_Z11_1 | 1 | 721476 | 89448868 | −0.1336 | 0.6086 | 2.0072 | 1.0423 | 2 | 1 |
| WCM14_Z11_1 | 1 | 89449069 | 91406840 | 0.2076 | 1.0328 | NA | NA | NA | NA |
| WCM14_Z11_1 | 1 | 91447883 | 92711097 | −0.1735 | 0.5572 | NA | NA | NA | NA |
| WCM14_Z11_1 | 1 | 92712203 | 102270469 | −0.4289 | 0.2181 | 1.359 | 0.9674 | 1 | 1 |
| WCM14_Z11_1 | 1 | 102271671 | 104080004 | −0.1871 | 0.5397 | NA | NA | NA | NA |
| WCM14_Z11_1 | 1 | 104084022 | 111738612 | −0.7935 | −0.308 | 1.5298 | 0.0857 | 2 | 0 |
| WCM14_Z11_1 | 1 | 111739874 | 119965099 | −0.3623 | 0.3085 | 1.3668 | 1.1101 | 1 | 1 |
| WCM14_Z11_1 | 1 | 119985626 | 197150225 | 0.0649 | 0.8583 | 2.5795 | 1.0463 | 3 | 1 |
| WCM14_Z11_1 | 1 | 197157513 | 207200888 | 0.3493 | 1.2024 | 2.364 | 2.2384 | 2 | 2 |
| WCM14_Z11_1 | 1 | 207222535 | 249212424 | −0.1723 | 0.5588 | 2.6236 | 0.3226 | 3 | 0 |
| WCM14_Z11_1 | 10 | 225980 | 70482258 | −0.1827 | 0.5454 | 1.9352 | 0.9837 | 2 | 1 |
| WCM14_Z11_1 | 10 | 70496707 | 77818522 | 0.5741 | 1.4655 | 3.6962 | 1.8268 | 4 | 2 |
| WCM14_Z11_1 | 10 | 78084229 | 81371653 | 1.0815 | 2.0378 | NA | NA | NA | NA |
| WCM14_Z11_1 | 10 | 81372081 | 135381800 | −0.5264 | 0.0829 | 2.0708 | 0.0475 | 2 | 0 |
| WCM14_Z11_1 | 11 | 193151 | 48328093 | 0.0239 | 0.8074 | 2.7849 | 0.7152 | 3 | 1 |
| WCM14_Z11_1 | 11 | 48328327 | 56510600 | 0.0651 | 0.8586 | 1.8715 | 1.7549 | 2 | 2 |
| WCM14_Z11_1 | 11 | 56756798 | 134856562 | 0.0864 | 0.8848 | 2.8931 | 0.8 | 3 | 1 |
| WCM14_Z11_1 | 12 | 148674 | 4737849 | 0.0673 | 0.8612 | 1.9086 | 1.7245 | 2 | 2 |
| WCM14_Z11_1 | 12 | 4758290 | 39161492 | −0.1786 | 0.5506 | 2.0516 | 0.8778 | 2 | 1 |
| WCM14_Z11_1 | 12 | 39242372 | 47471439 | 0.033 | 0.8187 | 1.8427 | 1.6849 | 2 | 2 |
| WCM14_Z11_1 | 12 | 47471567 | 64609742 | 0.1633 | 0.979 | 1.9782 | 1.9638 | 2 | 2 |
| WCM14_Z11_1 | 12 | 64616006 | 91347928 | 0.0838 | 0.8817 | 1.9424 | 1.7426 | 2 | 2 |
| WCM14_Z11_1 | 12 | 91348155 | 133810880 | 0.5575 | 1.4463 | 3.567 | 1.883 | 4 | 2 |
| WCM14_Z11_1 | 13 | 19601063 | 45841502 | −0.5714 | 0.0193 | 2.0244 | 0.0026 | 2 | 0 |
| WCM14_Z11_1 | 13 | 45857620 | 111896592 | −1.0766 | −0.7675 | 1.1198 | 0.055 | 1 | 0 |
| WCM14_Z11_1 | 13 | 111917932 | 115089411 | −0.5388 | 0.0655 | 2.1099 | −0.017 | 2 | 0 |
| WCM14_Z11_1 | 14 | 19377698 | 27064664 | 0.2143 | 1.0409 | 2.0848 | 2.0301 | 2 | 2 |
| WCM14_Z11_1 | 14 | 27065792 | 65008406 | 0.0936 | 0.8936 | 2.1162 | 1.5995 | 2 | 2 |
| WCM14_Z11_1 | 14 | 65008685 | 107283074 | 0.2087 | 1.034 | 2.07 | 2.0254 | 2 | 2 |
| WCM14_Z11_1 | 15 | 20170150 | 22466489 | −0.6466 | −0.0888 | 1.3715 | 0.5091 | 1 | 1 |
| WCM14_Z11_1 | 15 | 22482966 | 38234397 | −0.8998 | −0.4742 | 1.0759 | 0.3639 | 1 | 0 |
| WCM14_Z11_1 | 15 | 38235545 | 102515352 | −0.4606 | 0.1745 | 1.2905 | 0.9666 | 1 | 1 |
| WCM14_Z11_1 | 16 | 65607 | 33961499 | 0.1412 | 0.9521 | 1.9347 | 1.9347 | 2 | 2 |
| WCM14_Z11_1 | 16 | 33961702 | 80584447 | −0.145 | 0.594 | 1.9804 | 1.0385 | 2 | 1 |
| WCM14_Z11_1 | 16 | 80638372 | 82197800 | −1.0043 | −0.6445 | 1.1931 | 0.0863 | 1 | 0 |
| WCM14_Z11_1 | 16 | 82660694 | 90233294 | −0.1743 | 0.5563 | 1.9376 | 1.0033 | 2 | 1 |
| WCM14_Z11_1 | 17 | 131595 | 5463107 | 0.1278 | 0.9357 | 1.9175 | 1.9081 | 2 | 2 |
| WCM14_Z11_1 | 17 | 5463309 | 21191346 | −0.5124 | 0.1026 | 2.0923 | 0.0552 | 2 | 0 |
| WCM14_Z11_1 | 17 | 21194843 | 81083525 | 0.1413 | 0.9522 | 1.9442 | 1.9253 | 2 | 2 |
| WCM14_Z11_1 | 18 | 48259 | 19438557 | −0.1641 | 0.5694 | 1.9831 | 0.9848 | 2 | 1 |
| WCM14_Z11_1 | 18 | 19751342 | 78005201 | −0.5267 | 0.0825 | 2.0674 | 0.0504 | 2 | 0 |
| WCM14_Z11_1 | 19 | 281439 | 7833861 | −0.5221 | 0.089 | 2.0663 | 0.0609 | 2 | 0 |
| WCM14_Z11_1 | 19 | 7911442 | 32083584 | 0.1779 | 0.9967 | 2.0173 | 1.9736 | 2 | 2 |
| WCM14_Z11_1 | 19 | 32083892 | 59083988 | −0.029 | 0.7412 | 2.0013 | 1.3419 | 2 | 1 |
| WCM14_Z11_1 | 2 | 41614 | 92129784 | −0.1973 | 0.5265 | 1.8198 | 1.061 | 2 | 1 |
| WCM14_Z11_1 | 2 | 95537342 | 131099795 | −0.0038 | 0.7728 | 1.7364 | 1.6808 | 2 | 2 |
| WCM14_Z11_1 | 2 | 131100690 | 242946589 | −0.198 | 0.5255 | 1.7327 | 1.1462 | 2 | 1 |
| WCM14_Z11_1 | 20 | 68363 | 21695381 | −0.0441 | 0.7223 | 1.9553 | 1.3443 | 2 | 1 |
| WCM14_Z11_1 | 20 | 22562674 | 25755672 | 0.223 | 1.0513 | 2.21 | 1.9349 | 2 | 2 |
| WCM14_Z11_1 | 20 | 26063602 | 30437652 | −0.0499 | 0.715 | NA | NA | NA | NA |
| WCM14_Z11_1 | 20 | 30438475 | 41101195 | −0.2739 | 0.4263 | 1.6521 | 1.0354 | 2 | 1 |
| WCM14_Z11_1 | 20 | 41306600 | 62904848 | 0.025 | 0.8088 | 1.8106 | 1.6929 | 2 | 2 |
| WCM14_Z11_1 | 21 | 9907304 | 16423152 | −0.6782 | −0.1351 | NA | NA | NA | NA |
| WCM14_Z11_1 | 21 | 17102741 | 48084207 | 0.1069 | 0.9101 | 1.9022 | 1.8561 | 2 | 2 |
| WCM14_Z11_1 | 22 | 16449708 | 51219138 | −0.5106 | 0.1051 | 2.1089 | 0.0422 | 2 | 0 |
| WCM14_Z11_1 | 3 | 239436 | 7649779 | 0.2928 | 1.1352 | 2.7557 | 1.6374 | 3 | 2 |
| WCM14_Z11_1 | 3 | 7721939 | 15915340 | −0.502 | 0.1171 | 2.109 | 0.0602 | 2 | 0 |
| WCM14_Z11_1 | 3 | 16216731 | 32995599 | 0.3004 | 1.1443 | 2.6299 | 1.7908 | 3 | 2 |
| WCM14_Z11_1 | 3 | 32995928 | 41925382 | 0.04 | 0.8274 | 1.8053 | 1.7436 | 2 | 2 |
| WCM14_Z11_1 | 3 | 41938332 | 45636376 | 0.3728 | 1.2302 | 2.5458 | 2.1463 | 3 | 2 |
| WCM14_Z11_1 | 3 | 45636700 | 49298139 | −0.4941 | 0.1281 | 2.1153 | 0.0705 | 2 | 0 |
| WCM14_Z11_1 | 3 | 49298484 | 49751596 | −1.0114 | −0.6565 | 1.2541 | 0.0147 | 1 | 0 |
| WCM14_Z11_1 | 3 | 49753099 | 52658955 | −0.0969 | 0.6555 | NA | NA | NA | NA |
| WCM14_Z11_1 | 3 | 52661350 | 53836179 | −0.9303 | −0.5231 | NA | NA | NA | NA |
| WCM14_Z11_1 | 3 | 53837510 | 54157561 | −0.1619 | 0.5722 | NA | NA | NA | NA |
| WCM14_Z11_1 | 3 | 54354552 | 59999828 | 0.4812 | 1.3576 | 2.5626 | 2.5626 | 3 | 3 |
| WCM14_Z11_1 | 3 | 60522689 | 85984919 | −0.152 | 0.585 | 3.0237 | −0.0236 | 3 | 0 |
| WCM14_Z11_1 | 3 | 86010623 | 113441818 | 0.0442 | 0.8326 | 1.8317 | 1.7301 | 2 | 2 |
| WCM14_Z11_1 | 3 | 113442385 | 125313565 | −0.4971 | 0.124 | NA | NA | NA | NA |
| WCM14_Z11_1 | 3 | 125649401 | 128526493 | 0.4175 | 1.283 | 2.9372 | 1.9298 | 3 | 2 |
| WCM14_Z11_1 | 3 | 128532190 | 142775178 | 0.1357 | 0.9453 | 1.9727 | 1.8785 | 2 | 2 |
| WCM14_Z11_1 | 3 | 142839762 | 180359919 | 0.2553 | 1.0902 | 2.3848 | 1.8733 | 2 | 2 |
| WCM14_Z11_1 | 3 | 180361955 | 184042761 | −0.148 | 0.5901 | 2.0275 | 0.9832 | 2 | 1 |
| WCM14_Z11_1 | 3 | 184043303 | 197894712 | 0.1402 | 0.9508 | 2.0345 | 1.8314 | 2 | 2 |
| WCM14_Z11_1 | 4 | 59327 | 57899380 | −0.1667 | 0.566 | 1.9386 | 1.0223 | 2 | 1 |
| WCM14_Z11_1 | 4 | 57907036 | 83838806 | 0.2686 | 1.1062 | 2.824 | 1.4816 | 3 | 1 |
| WCM14_Z11_1 | 4 | 83839054 | 84028971 | 0.0731 | 0.8684 | NA | NA | NA | NA |
| WCM14_Z11_1 | 4 | 84185374 | 151773593 | −0.1714 | 0.5599 | 1.9366 | 1.0118 | 2 | 1 |
| WCM14_Z11_1 | 4 | 151773951 | 190426570 | 0.0728 | 0.868 | 1.868 | 1.7824 | 2 | 2 |
| WCM14_Z11_1 | 5 | 140682 | 49699117 | 0.2255 | 1.0544 | 2.1244 | 2.0292 | 2 | 2 |
| WCM14_Z11_1 | 5 | 49706728 | 180687769 | −0.166 | 0.5669 | 2.9462 | 0.0164 | 3 | 0 |
| WCM14_Z11_1 | 6 | 203561 | 62407127 | −0.1453 | 0.5935 | 1.972 | 1.0459 | 2 | 1 |
| WCM14_Z11_1 | 6 | 62442604 | 69349121 | −1.1824 | −0.9556 | NA | NA | NA | NA |
| WCM14_Z11_1 | 6 | 69640532 | 110636619 | −0.2034 | 0.5185 | 1.9237 | 0.9411 | 2 | 1 |
| WCM14_Z11_1 | 6 | 110643995 | 135239816 | 0.0896 | 0.8887 | 2.8137 | 0.8893 | 3 | 1 |
| WCM14_Z11_1 | 6 | 135250239 | 149068711 | −0.1669 | 0.5658 | 1.967 | 0.9935 | 2 | 1 |
| WCM14_Z11_1 | 6 | 149208150 | 152673418 | −1.0773 | −0.7687 | 1.0998 | 0.0741 | 1 | 0 |
| WCM14_Z11_1 | 6 | 152674455 | 158834221 | −0.0906 | 0.6634 | 2.0165 | 1.1512 | 2 | 1 |
| WCM14_Z11_1 | 6 | 158850844 | 159688951 | −1.1042 | −0.8155 | 1.0486 | 0.0878 | 1 | 0 |
| WCM14_Z11_1 | 6 | 160103542 | 166580882 | −0.0872 | 0.6678 | 1.873 | 1.3042 | 2 | 1 |
| WCM14_Z11_1 | 6 | 166720806 | 170893661 | −0.5573 | 0.0394 | 2.0223 | 0.0331 | 2 | 0 |
| WCM14_Z11_1 | 7 | 193265 | 38785175 | −0.157 | 0.5785 | 1.9699 | 1.0167 | 2 | 1 |
| WCM14_Z11_1 | 7 | 38791817 | 40174670 | 1.2752 | 2.2503 | NA | NA | NA | NA |
| WCM14_Z11_1 | 7 | 40220595 | 56130404 | 0.1108 | 0.9149 | 1.9317 | 1.8391 | 2 | 2 |
| WCM14_Z11_1 | 7 | 56130723 | 158937268 | 0.3548 | 1.209 | 2.8571 | 1.7664 | 3 | 2 |
| WCM14_Z11_1 | 8 | 190906 | 43212022 | −0.5221 | 0.0889 | 2.0783 | 0.0489 | 2 | 0 |
| WCM14_Z11_1 | 8 | 47752745 | 146279480 | 0.8941 | 1.8294 | 5.346 | 1.7618 | 5 | 2 |
| WCM14_Z11_1 | 9 | 17040 | 17579284 | −1.06 | −0.7388 | 1.1573 | 0.0411 | 1 | 0 |
| WCM14_Z11_1 | 9 | 17747099 | 20823070 | 1.0279 | 1.9785 | NA | NA | NA | NA |
| WCM14_Z11_1 | 9 | 20862634 | 32567188 | −0.1173 | 0.6294 | NA | NA | NA | NA |
| WCM14_Z11_1 | 9 | 32567414 | 32635509 | 0.1886 | 1.0098 | NA | NA | NA | NA |
| WCM14_Z11_1 | 9 | 32784303 | 34256832 | 0.5149 | 1.3969 | 3.6192 | 1.6474 | 4 | 2 |
| WCM14_Z11_1 | 9 | 34257095 | 71395287 | 0.1588 | 0.9736 | 2.3847 | 1.5427 | 2 | 2 |
| WCM14_Z11_1 | 9 | 71395435 | 141111414 | −0.1508 | 0.5865 | 2.9454 | 0.0578 | 3 | 0 |
| WCM14_Z11_1 | X | 2700148 | 65392239 | 0.7989 | 1.7222 | NA | NA | NA | NA |
| WCM14_Z11_1 | X | 65393522 | 68060476 | 1.7319 | 2.7419 | NA | NA | NA | NA |
| WCM14_Z11_1 | X | 68381398 | 92964855 | −0.5647 | 0.0289 | NA | NA | NA | NA |
| WCM14_Z11_1 | X | 95940147 | 154509348 | −0.0441 | 0.7222 | NA | NA | NA | NA |
| WCM15 | 1 | 14907 | 10428452 | 0.05 | 0.7375 | 1.6673 | 1.6673 | 2 | 2 |
| WCM15 | 1 | 10431158 | 12780953 | −0.1663 | −0.9906 | 0.9638 | 0.0427 | 1 | 0 |
| WCM15 | 1 | 12785247 | 249213964 | 0.0283 | 0.625 | 1.5422 | 1.5422 | 2 | 2 |
| WCM15 | 10 | 226093 | 88649941 | −0.0013 | 0.4593 | 1.3749 | 1.3749 | 1 | 1 |
| WCM15 | 10 | 88650021 | 95326754 | −0.315 | NA | −0.1039 | −0.2987 | 0 | 0 |
| WCM15 | 10 | 95335903 | 135477891 | 0.0328 | 0.6489 | 1.568 | 1.568 | 2 | 2 |
| WCM15 | 11 | 180151 | 134605849 | 0.0068 | 0.5062 | 1.4203 | 1.4203 | 1 | 1 |
| WCM15 | 12 | 148465 | 13768448 | −0.1521 | −0.7994 | 0.7708 | 0.3784 | 1 | 0 |
| WCM15 | 12 | 13769404 | 133448825 | 0.0402 | 0.6877 | 1.6107 | 1.6107 | 2 | 2 |
| WCM15 | 12 | 133454126 | 133810842 | −0.1835 | −1.2576 | NA | NA | NA | NA |
| WCM15 | 13 | 19042004 | 21753162 | −0.1376 | −0.6265 | 1.0294 | 0.2661 | 1 | 0 |
| WCM15 | 13 | 21875699 | 48830390 | 0.203 | 1.3799 | 5.6698 | −0.4648 | 6 | 0 |
| WCM15 | 13 | 48832327 | 115089411 | −0.1923 | −1.4161 | 0.5231 | 0.2263 | 1 | 0 |
| WCM15 | 14 | 19110194 | 107283210 | 0.2422 | 1.5153 | 2.8587 | 2.8587 | 3 | 3 |
| WCM15 | 15 | 20083857 | 59519598 | −0.0869 | −0.1351 | 0.9106 | 0.9106 | 1 | 1 |
| WCM15 | 15 | 59523887 | 101445757 | 0.0212 | 0.5868 | 1.5265 | 1.4774 | 2 | 1 |
| WCM15 | 15 | 101447450 | 102514189 | −0.1104 | −0.3442 | 1.3338 | 0.2417 | 1 | 0 |
| WCM15 | 16 | 66888 | 90168693 | −0.1869 | −1.3171 | 0.678 | 0.1247 | 1 | 0 |
| WCM15 | 17 | 131595 | 16089921 | 0.0048 | 0.495 | 3.2727 | −0.4541 | 3 | 0 |
| WCM15 | 17 | 16090076 | 21201579 | −0.2245 | −2.1914 | 0.6682 | −0.2303 | 1 | 0 |
| WCM15 | 17 | 21201851 | 81187883 | 0.0461 | 0.7176 | 1.6445 | 1.6445 | 2 | 2 |
| WCM15 | 18 | 47806 | 78005197 | −0.0721 | −0.0165 | 2.3789 | −0.4016 | 2 | 0 |
| WCM15 | 19 | 105010 | 11411925 | 0.065 | 0.8112 | 1.7547 | 1.7547 | 2 | 2 |
| WCM15 | 19 | 11417438 | 14655988 | −0.2488 | −3.2717 | 0.2599 | −0.0528 | 0 | 0 |
| WCM15 | 19 | 14673353 | 59093696 | 0.1913 | 1.3376 | 2.5272 | 2.5272 | 3 | 3 |
| WCM15 | 2 | 41614 | 243037022 | −0.0701 | −0.001 | 0.9993 | 0.9993 | 1 | 1 |
| WCM15 | 20 | 68375 | 25657632 | −0.1116 | −0.3562 | 1.9545 | −0.3921 | 2 | 0 |
| WCM15 | 20 | 25666249 | 62934623 | 0.1408 | 1.1444 | 2.2105 | 2.2105 | 2 | 2 |
| WCM15 | 21 | 9590311 | 48084181 | 0.0366 | 0.6691 | 1.5901 | 1.5901 | 2 | 2 |
| WCM15 | 22 | 16159014 | 51219089 | −0.17 | −1.0447 | 0.6498 | 0.3197 | 1 | 0 |
| WCM15 | 3 | 239436 | 197896662 | 0.0601 | 0.7873 | 1.7258 | 1.7258 | 2 | 2 |
| WCM15 | 4 | 53346 | 77228138 | −0.1405 | −0.66 | 0.6329 | 0.6329 | 1 | 1 |
| WCM15 | 4 | 77230296 | 85416804 | −0.3233 | NA | −0.2387 | −0.2387 | 0 | 0 |
| WCM15 | 4 | 85417019 | 190878508 | −0.0857 | −0.1257 | 0.9166 | 0.9166 | 1 | 1 |
| WCM15 | 5 | 90750 | 50125712 | 0.2346 | 1.4895 | 2.808 | 2.808 | 3 | 3 |
| WCM15 | 5 | 50128684 | 148016583 | −0.0814 | −0.0905 | 2.4426 | −0.5642 | 2 | −1 |
| WCM15 | 5 | 148206440 | 180687808 | 0.2526 | 1.5497 | 2.9276 | 2.9276 | 3 | 3 |
| WCM15 | 6 | 203428 | 19838753 | 0.0217 | 0.5898 | 1.505 | 1.505 | 2 | 2 |
| WCM15 | 6 | 19839429 | 28334474 | −0.2377 | −2.6809 | 0.2998 | 0.0121 | 0 | 0 |
| WCM15 | 6 | 28336407 | 170892860 | 0.0449 | 0.7116 | 1.6377 | 1.6377 | 2 | 2 |
| WCM15 | 7 | 193254 | 16823442 | −0.1831 | −1.2518 | 0.6191 | 0.2208 | 1 | 0 |
| WCM15 | 7 | 16823775 | 130294942 | −0.002 | 0.4556 | 1.3714 | 1.3714 | 1 | 1 |
| WCM15 | 7 | 130295322 | 132973648 | −0.021 | 0.3403 | 1.274 | 1.2581 | 1 | 1 |
| WCM15 | 7 | 132973904 | 158738404 | 0.1651 | 1.2397 | 2.5335 | 2.1894 | 3 | 2 |
| WCM15 | 7 | 158738484 | 158937158 | −0.098 | −0.2305 | NA | NA | NA | NA |
| WCM15 | 8 | 163457 | 32152225 | −0.1867 | −1.3134 | 0.5514 | 0.2534 | 1 | 0 |
| WCM15 | 8 | 32406215 | 145933686 | 0.0347 | 0.6589 | 1.5789 | 1.5789 | 2 | 2 |
| WCM15 | 8 | 145946992 | 146279510 | −0.201 | −1.5891 | NA | NA | NA | NA |
| WCM15 | 9 | 14856 | 39178192 | −0.0614 | 0.0651 | 1.0462 | 1.0462 | 1 | 1 |
| WCM15 | 9 | 39817370 | 77752528 | −0.3065 | NA | 0.0982 | −0.4242 | 0 | 0 |
| WCM15 | 9 | 77755768 | 141107513 | −0.0198 | 0.3476 | 1.2724 | 1.2724 | 1 | 1 |
| WCM15 | X | 2700162 | 26213604 | 0.3771 | 1.9241 | NA | NA | NA | NA |
| WCM15 | X | 26235651 | 154509348 | 0.0595 | 0.7846 | NA | NA | NA | NA |
| WCM154 | 1 | 14907 | 16817677 | −0.2964 | 0.1971 | 2.1785 | 0.1143 | 2 | 0 |
| WCM154 | 1 | 16828222 | 17198659 | −0.4363 | −0.1492 | 1.555 | 0.2485 | 2 | 0 |
| WCM154 | 1 | 17248500 | 28010508 | −0.3374 | 0.1007 | 2.1192 | 0.0254 | 2 | 0 |
| WCM154 | 1 | 28014988 | 51253917 | 0.3427 | 1.3903 | 2.6214 | 2.6214 | 3 | 3 |
| WCM154 | 1 | 51267373 | 55452078 | 0.0126 | 0.8308 | 1.9294 | 1.6278 | 2 | 2 |
| WCM154 | 1 | 55457489 | 72739681 | 0.3286 | 1.3682 | 2.5855 | 2.5773 | 3 | 3 |
| WCM154 | 1 | 72748016 | 107682908 | −0.0922 | 0.6312 | 3.0427 | 0.0549 | 3 | 0 |
| WCM154 | 1 | 107683065 | 225339733 | 0.3308 | 1.3717 | 2.5918 | 2.5836 | 3 | 3 |
| WCM154 | 1 | 225340546 | 226259090 | −0.1489 | 0.5173 | NA | NA | NA | NA |
| WCM154 | 1 | 226332498 | 249213973 | 0.2986 | 1.3203 | 2.4973 | 2.4973 | 2 | 2 |
| WCM154 | 10 | 225989 | 135477891 | −0.3536 | 0.0616 | 2.0356 | 0.0516 | 2 | 0 |
| WCM154 | 11 | 193112 | 134605959 | −0.195 | 0.4213 | 1.3391 | 1.3391 | 1 | 1 |
| WCM154 | 12 | 148444 | 29519813 | −0.3431 | 0.0869 | 2.1074 | 0.0167 | 2 | 0 |
| WCM154 | 12 | 29521193 | 120936081 | 0.0938 | 0.977 | 1.9684 | 1.9684 | 2 | 2 |
| WCM154 | 12 | 120941464 | 132212822 | 0.3691 | 1.4316 | 2.7563 | 2.6388 | 3 | 3 |
| WCM154 | 12 | 132237750 | 133810883 | 0.2137 | 1.182 | 2.367 | 2.171 | 2 | 2 |
| WCM154 | 13 | 19041979 | 36424881 | 0.3728 | 1.4374 | 3.9119 | 1.5047 | 4 | 2 |
| WCM154 | 13 | 36428595 | 55015114 | 0.0688 | 0.9326 | 3.8789 | −0.0615 | 4 | 0 |
| WCM154 | 13 | 55015454 | 115089411 | 0.3268 | 1.3653 | 3.7339 | 1.4186 | 4 | 1 |
| WCM154 | 14 | 19110183 | 107283263 | 0.136 | 1.0506 | 2.0714 | 2.0714 | 2 | 2 |
| WCM154 | 15 | 20083885 | 23811262 | −0.2152 | 0.3782 | 1.9801 | 0.6194 | 2 | 1 |
| WCM154 | 15 | 23811592 | 102506273 | −0.1352 | 0.5452 | 2.8176 | 0.101 | 3 | 0 |
| WCM154 | 16 | 67604 | 2581992 | −0.2473 | 0.308 | 2.3605 | 0.1154 | 2 | 0 |
| WCM154 | 16 | 2593705 | 33965560 | 0.1931 | 1.1475 | 2.2153 | 2.2153 | 2 | 2 |
| WCM154 | 16 | 34274675 | 90168697 | −0.3378 | 0.0998 | 2.1183 | 0.025 | 2 | 0 |
| WCM154 | 17 | 131595 | 440373 | 0.126 | 1.0332 | NA | NA | NA | NA |
| WCM154 | 17 | 455146 | 17210003 | −0.3365 | 0.1029 | 2.137 | 0.0109 | 2 | 0 |
| WCM154 | 17 | 17226544 | 80038011 | −0.0515 | 0.7103 | 2.0187 | 1.2535 | 2 | 1 |
| WCM154 | 17 | 80038478 | 81052563 | 0.189 | 1.1408 | 2.205 | 2.205 | 2 | 2 |
| WCM154 | 18 | 47800 | 78005123 | 0.1581 | 1.0884 | 2.1263 | 2.1263 | 2 | 2 |
| WCM154 | 19 | 104935 | 59093696 | 0.1303 | 1.0406 | 2.0571 | 2.0571 | 2 | 2 |
| WCM154 | 2 | 41662 | 69300093 | −0.062 | 0.6901 | 1.8874 | 1.3394 | 2 | 1 |
| WCM154 | 2 | 69302835 | 243037022 | −0.346 | 0.08 | 2.0542 | 0.0598 | 2 | 0 |
| WCM154 | 20 | 68351 | 62934623 | −0.0511 | 0.7109 | 1.6368 | 1.6368 | 2 | 2 |
| WCM154 | 21 | 9590331 | 34030022 | −0.3656 | 0.032 | 1.8851 | 0.1598 | 2 | 0 |
| WCM154 | 21 | 34037433 | 42839671 | 0.2375 | 1.2213 | 2.3815 | 2.2818 | 2 | 2 |
| WCM154 | 21 | 42839878 | 48084211 | −0.3224 | 0.1365 | 2.1623 | 0.0361 | 2 | 0 |
| WCM154 | 22 | 16159043 | 51223730 | −0.3021 | 0.1839 | 1.7712 | 0.5007 | 2 | 1 |
| WCM154 | 3 | 239431 | 90279242 | 0.0676 | 0.9306 | 1.906 | 1.906 | 2 | 2 |
| WCM154 | 3 | 90283452 | 107097091 | −0.3843 | −0.0146 | 1.9784 | 0.0014 | 2 | 0 |
| WCM154 | 3 | 107097387 | 185641782 | 0.3326 | 1.3745 | 2.6501 | 2.5355 | 3 | 3 |
| WCM154 | 3 | 185643220 | 197896740 | 0.113 | 1.0107 | 2.0148 | 2.0148 | 2 | 2 |
| WCM154 | 4 | 53435 | 184828290 | −0.3747 | 0.0096 | 1.0067 | 1.0067 | 1 | 1 |
| WCM154 | 4 | 184910040 | 190874234 | −0.763 | −1.2684 | 0.9092 | −0.079 | 1 | 0 |
| WCM154 | 5 | 90537 | 34043227 | 0.3678 | 1.4296 | 2.7608 | 2.6266 | 3 | 3 |
| WCM154 | 5 | 34175975 | 34193028 | −0.7179 | −1.0711 | NA | NA | NA | NA |
| WCM154 | 5 | 34526264 | 64981293 | 0.3506 | 1.4027 | 2.7517 | 2.5362 | 3 | 3 |
| WCM154 | 5 | 65000283 | 74018300 | −0.3909 | −0.0312 | 1.9799 | −0.0228 | 2 | 0 |
| WCM154 | 5 | 74021755 | 82406889 | 0.38 | 1.4485 | 2.7969 | 2.6616 | 3 | 3 |
| WCM154 | 5 | 82491751 | 90136848 | −0.4045 | −0.066 | 1.8294 | 0.0812 | 2 | 0 |
| WCM154 | 5 | 90144522 | 180687814 | 0.3506 | 1.4028 | 2.6855 | 2.6026 | 3 | 3 |
| WCM154 | 6 | 203364 | 78633079 | 0.0839 | 0.9596 | 1.9447 | 1.9447 | 2 | 2 |
| WCM154 | 6 | 79424459 | 102516718 | −0.3867 | −0.0205 | 1.9977 | −0.0259 | 2 | 0 |
| WCM154 | 6 | 105176968 | 170892855 | 0.1002 | 0.9882 | 1.9837 | 1.9837 | 2 | 2 |
| WCM154 | 7 | 193222 | 158937066 | 0.0845 | 0.9605 | 1.9459 | 1.9459 | 2 | 2 |
| WCM154 | 8 | 163532 | 146279533 | 0.1479 | 1.0709 | 2.1007 | 2.1007 | 2 | 2 |
| WCM154 | 9 | 14878 | 79007527 | 0.0868 | 0.9647 | 1.9516 | 1.9516 | 2 | 2 |
| WCM154 | 9 | 79009077 | 86905107 | −0.3596 | 0.0467 | 1.9372 | 0.1287 | 2 | 0 |
| WCM154 | 9 | 86907582 | 93657870 | 0.1431 | 1.0627 | 2.0888 | 2.0888 | 2 | 2 |
| WCM154 | 9 | 93976491 | 95571939 | −0.3309 | 0.1162 | 2.2337 | −0.0659 | 2 | 0 |
| WCM154 | 9 | 95575820 | 95609878 | 0.2736 | 1.2802 | NA | NA | NA | NA |
| WCM154 | 9 | 95610230 | 130104684 | 0.1447 | 1.0654 | 2.0928 | 2.0928 | 2 | 2 |
| WCM154 | 9 | 130106435 | 141107513 | 0.2657 | 1.2673 | 2.4072 | 2.4072 | 2 | 2 |
| WCM154 | X | 2700159 | 9656265 | 0.1244 | 1.0305 | NA | NA | NA | NA |
| WCM154 | X | 9659738 | 38547899 | 0.0821 | 0.9563 | NA | NA | NA | NA |
| WCM154 | X | 38664196 | 39935801 | −1.1843 | NA | NA | NA | NA | NA |
| WCM154 | X | 40218561 | 49034387 | 0.0419 | 0.8842 | NA | NA | NA | NA |
| WCM154 | X | 49065850 | 154493598 | 0.2205 | 1.1933 | NA | NA | NA | NA |
| WCM154_Z1_1 | 1 | 69603 | 16802942 | −0.5794 | 0.2166 | 2.2971 | 0.027 | 2 | 0 |
| WCM154_Z1_1 | 1 | 16863328 | 17275305 | −1.017 | −0.3008 | 1.3949 | 0.2287 | 1 | 0 |
| WCM154_Z1_1 | 1 | 17277585 | 28052657 | −0.6148 | 0.1758 | 2.2097 | 0.0495 | 2 | 0 |
| WCM154_Z1_1 | 1 | 28054037 | 28290084 | 0.4196 | 1.3211 | NA | NA | NA | NA |
| WCM154_Z1_1 | 1 | 28293068 | 29365776 | −0.0475 | 0.8146 | NA | NA | NA | NA |
| WCM154_Z1_1 | 1 | 29379775 | 51253762 | 0.4547 | 1.3587 | 2.9099 | 2.2192 | 3 | 2 |
| WCM154_Z1_1 | 1 | 51267349 | 55307501 | −0.1104 | 0.7452 | 2.2894 | 1.0629 | 2 | 1 |
| WCM154_Z1_1 | 1 | 55318024 | 65312358 | 0.4229 | 1.3247 | 2.6093 | 2.4002 | 3 | 2 |
| WCM154_Z1_1 | 1 | 65313282 | 66384416 | −0.1602 | 0.6901 | NA | NA | NA | NA |
| WCM154_Z1_1 | 1 | 66458649 | 72748046 | 0.408 | 1.3087 | 2.5753 | 2.379 | 3 | 2 |
| WCM154_Z1_1 | 1 | 74492554 | 104163199 | −0.3211 | 0.5105 | 2.8141 | 0.0349 | 3 | 0 |
| WCM154_Z1_1 | 1 | 107599497 | 225340563 | 0.4077 | 1.3084 | 2.7792 | 2.1742 | 3 | 2 |
| WCM154_Z1_1 | 1 | 225341507 | 226187000 | −0.1851 | 0.6624 | NA | NA | NA | NA |
| WCM154_Z1_1 | 1 | 226253527 | 249212342 | 0.1802 | 1.0633 | 2.0896 | 2.0896 | 2 | 2 |
| WCM154_Z1_1 | 10 | 225958 | 135440068 | −0.7717 | −0.0072 | 1.9556 | 0.0344 | 2 | 0 |
| WCM154_Z1_1 | 11 | 193112 | 48328644 | −0.3469 | 0.4814 | 2.6825 | 0.1097 | 3 | 0 |
| WCM154_Z1_1 | 11 | 48346822 | 57148170 | −0.3921 | 0.4303 | 1.7087 | 0.9863 | 2 | 1 |
| WCM154_Z1_1 | 11 | 57154983 | 134856626 | −0.3491 | 0.4789 | 2.6968 | 0.0904 | 3 | 0 |
| WCM154_Z1_1 | 12 | 148642 | 29521193 | −0.6911 | 0.0872 | 2.0546 | 0.07 | 2 | 0 |
| WCM154_Z1_1 | 12 | 29523114 | 120128145 | 0.0792 | 0.9534 | 1.9617 | 1.911 | 2 | 2 |
| WCM154_Z1_1 | 12 | 120135573 | 120662505 | −0.256 | 0.5834 | NA | NA | NA | NA |
| WCM154_Z1_1 | 12 | 120720384 | 132238926 | 0.4243 | 1.3262 | 2.859 | 2.1558 | 3 | 2 |
| WCM154_Z1_1 | 12 | 132239988 | 132633366 | −0.212 | 0.6325 | NA | NA | NA | NA |
| WCM154_Z1_1 | 12 | 132635585 | 133810874 | 0.4275 | 1.3296 | 2.8872 | 2.1394 | 3 | 2 |
| WCM154_Z1_1 | 13 | 19600515 | 36429656 | 0.3632 | 1.2607 | 2.8669 | 1.9254 | 3 | 2 |
| WCM154_Z1_1 | 13 | 36445390 | 54886136 | −0.3066 | 0.5267 | 2.8379 | 0.0435 | 3 | 0 |
| WCM154_Z1_1 | 13 | 55748673 | 115089411 | 0.3527 | 1.2494 | 2.7554 | 1.9995 | 3 | 2 |
| WCM154_Z1_1 | 14 | 19377718 | 107283092 | 0.1044 | 0.9808 | 1.9908 | 1.9564 | 2 | 2 |
| WCM154_Z1_1 | 15 | 20170159 | 23807222 | −0.8031 | −0.0443 | 1.3366 | 0.603 | 1 | 1 |
| WCM154_Z1_1 | 15 | 23811118 | 102506284 | −0.7487 | 0.0197 | 1.985 | 0.0425 | 2 | 0 |
| WCM154_Z1_1 | 16 | 67604 | 2581992 | −0.6968 | 0.0805 | 2.0679 | 0.0469 | 2 | 0 |
| WCM154_Z1_1 | 16 | 2636684 | 34256879 | 0.1255 | 1.0038 | 2.0315 | 1.9791 | 2 | 2 |
| WCM154_Z1_1 | 16 | 34257210 | 90233626 | −0.6573 | 0.1265 | 2.1416 | 0.0416 | 2 | 0 |
| WCM154_Z1_1 | 17 | 131595 | 7559174 | 0.0704 | 0.9437 | 3.8114 | 0.0356 | 4 | 0 |
| WCM154_Z1_1 | 17 | 7573966 | 7579856 | −2.4614 | −2.4214 | NA | NA | NA | NA |
| WCM154_Z1_1 | 17 | 7580651 | 20931872 | 0.0759 | 0.9498 | 3.8141 | 0.0491 | 4 | 0 |
| WCM154_Z1_1 | 17 | 20946100 | 21454339 | 0.1464 | 1.0266 | 2.1678 | 1.9066 | 2 | 2 |
| WCM154_Z1_1 | 17 | 21454552 | 78237495 | 0.0707 | 0.9441 | 3.7934 | 0.0546 | 4 | 0 |
| WCM154_Z1_1 | 17 | 78247122 | 80019506 | 0.423 | 1.3248 | 4.02 | 0.99 | 4 | 1 |
| WCM154_Z1_1 | 17 | 80019857 | 81083526 | 0.6458 | 1.562 | 3.8101 | 2.0953 | 4 | 2 |
| WCM154_Z1_1 | 18 | 48233 | 78005214 | 0.604 | 1.5177 | 2.8634 | 2.8634 | 3 | 3 |
| WCM154_Z1_1 | 19 | 281491 | 59083951 | 0.1144 | 0.9917 | 2.0037 | 1.9734 | 2 | 2 |
| WCM154_Z1_1 | 2 | 41614 | 69240666 | 0.0504 | 0.9219 | 2.8121 | 0.9771 | 3 | 1 |
| WCM154_Z1_1 | 2 | 69267173 | 242946607 | −0.2925 | 0.5425 | 2.8756 | 0.0375 | 3 | 0 |
| WCM154_Z1_1 | 20 | 68351 | 62904881 | −0.1539 | 0.6971 | 2.085 | 1.1574 | 2 | 1 |
| WCM154_Z1_1 | 21 | 9907280 | 34017281 | −0.3865 | 0.4367 | 2.6784 | 0.0285 | 3 | 0 |
| WCM154_Z1_1 | 21 | 34017994 | 34113830 | 0.1174 | 0.995 | NA | NA | NA | NA |
| WCM154_Z1_1 | 21 | 34115350 | 39817513 | 0.5473 | 1.4574 | 2.7842 | 2.7082 | 3 | 3 |
| WCM154_Z1_1 | 21 | 39822803 | 42839766 | 0.3007 | 1.1935 | 2.9916 | 1.5825 | 3 | 2 |
| WCM154_Z1_1 | 21 | 42840423 | 48084214 | −0.341 | 0.488 | 2.7636 | 0.0414 | 3 | 0 |
| WCM154_Z1_1 | 22 | 16287443 | 24313782 | −0.2118 | 0.6326 | 3.0804 | 0.0204 | 3 | 0 |
| WCM154_Z1_1 | 22 | 24340851 | 24432609 | 0.8663 | 1.7947 | NA | NA | NA | NA |
| WCM154_Z1_1 | 22 | 24434805 | 51219138 | −0.2195 | 0.6241 | 3.0356 | 0.0468 | 3 | 0 |
| WCM154_Z1_1 | 3 | 239377 | 89528644 | 0.0196 | 0.8883 | 1.8854 | 1.8165 | 2 | 2 |
| WCM154_Z1_1 | 3 | 93595840 | 105588192 | −0.8046 | −0.0461 | 1.9178 | 0.0193 | 2 | 0 |
| WCM154_Z1_1 | 3 | 107096547 | 185638927 | 0.3436 | 1.2397 | 2.7878 | 1.9351 | 3 | 2 |
| WCM154_Z1_1 | 3 | 185641632 | 197894712 | −0.0246 | 0.8398 | 1.8055 | 1.774 | 2 | 2 |
| WCM154_Z1_1 | 4 | 59434 | 184633780 | −0.7804 | −0.0175 | 1.1207 | 0.8552 | 1 | 1 |
| WCM154_Z1_1 | 4 | 184827971 | 190426502 | −1.4807 | −0.8886 | 1.006 | 0.0743 | 1 | 0 |
| WCM154_Z1_1 | 5 | 140621 | 65004313 | 0.7894 | 1.7139 | 4.6838 | 1.8769 | 5 | 2 |
| WCM154_Z1_1 | 5 | 65008792 | 74021487 | −0.7731 | −0.0089 | 1.9629 | 0.0248 | 2 | 0 |
| WCM154_Z1_1 | 5 | 74021874 | 82373138 | 0.7958 | 1.7205 | 4.6948 | 1.8963 | 5 | 2 |
| WCM154_Z1_1 | 5 | 82406962 | 90136712 | −0.7972 | −0.0374 | 1.9067 | 0.0422 | 2 | 0 |
| WCM154_Z1_1 | 5 | 90144496 | 180687814 | 0.7897 | 1.7141 | 4.6604 | 1.9014 | 5 | 2 |
| WCM154_Z1_1 | 6 | 203615 | 78173107 | 0.0614 | 0.934 | 1.9523 | 1.8688 | 2 | 2 |
| WCM154_Z1_1 | 6 | 79424433 | 102516260 | −0.6983 | 0.0788 | 2.0792 | 0.0331 | 2 | 0 |
| WCM154_Z1_1 | 6 | 105177568 | 170893407 | 0.0407 | 0.9114 | 2.066 | 1.6957 | 2 | 2 |
| WCM154_Z1_1 | 7 | 193254 | 158937018 | −0.226 | 0.6169 | 2.0688 | 0.9983 | 2 | 1 |
| WCM154_Z1_1 | 8 | 190906 | 146279453 | 0.3026 | 1.1955 | 2.6775 | 1.903 | 3 | 2 |
| WCM154_Z1_1 | 9 | 17088 | 78965806 | 0.0567 | 0.9288 | 2.6485 | 1.159 | 3 | 1 |
| WCM154_Z1_1 | 9 | 78969059 | 86900926 | −0.364 | 0.4621 | 2.7419 | 0.0131 | 3 | 0 |
| WCM154_Z1_1 | 9 | 86903030 | 93657848 | 0.2809 | 1.1721 | 2.8293 | 1.6775 | 3 | 2 |
| WCM154_Z1_1 | 9 | 93976701 | 95608658 | −0.3483 | 0.4798 | 2.7912 | −0.0021 | 3 | 0 |
| WCM154_Z1_1 | 9 | 95608768 | 95874189 | 0.4449 | 1.3483 | NA | NA | NA | NA |
| WCM154_Z1_1 | 9 | 95874528 | 130083043 | 0.3075 | 1.2008 | 2.7986 | 1.7987 | 3 | 2 |
| WCM154_Z1_1 | 9 | 130087356 | 141111414 | 0.3918 | 1.2914 | 2.7652 | 2.1301 | 3 | 2 |
| WCM154_Z1_1 | X | 2707711 | 38664643 | 0.0263 | 0.8956 | NA | NA | NA | NA |
| WCM154_Z1_1 | X | 39696863 | 40450579 | −2.3745 | −2.2569 | NA | NA | NA | NA |
| WCM154_Z1_1 | X | 40456505 | 154509348 | 0.0915 | 0.9668 | NA | NA | NA | NA |
| WCM155 | 1 | 17453 | 22987613 | −0.37 | 0.1586 | 1.1162 | 1.1162 | 1 | 1 |
| WCM155 | 1 | 22987765 | 26131804 | −1.1442 | −0.9594 | 0.9991 | 0.0294 | 1 | 0 |
| WCM155 | 1 | 26135250 | 31192678 | −0.2998 | 0.2492 | 1.2159 | 1.1611 | 1 | 1 |
| WCM155 | 1 | 31194308 | 249213973 | 0.2303 | 0.8986 | 2.6993 | 1.0292 | 3 | 1 |
| WCM155 | 10 | 225936 | 2056415 | 0.3275 | 1.0125 | NA | NA | NA | NA |
| WCM155 | 10 | 3111625 | 57359679 | −0.2476 | 0.3157 | 2.4044 | 0.0848 | 2 | 0 |
| WCM155 | 10 | 58117959 | 60124557 | −2.4118 | −5.7406 | NA | NA | NA | NA |
| WCM155 | 10 | 60127688 | 61554414 | −0.2768 | 0.2785 | NA | NA | NA | NA |
| WCM155 | 10 | 61564204 | 88760258 | 0.1477 | 0.8007 | 2.4289 | 1.055 | 2 | 1 |
| WCM155 | 10 | 88761503 | 89603915 | −0.1829 | 0.3971 | NA | NA | NA | NA |
| WCM155 | 10 | 89621724 | 90074395 | −2.1402 | −3.448 | NA | NA | NA | NA |
| WCM155 | 10 | 90122345 | 135477904 | −0.2068 | 0.3672 | 2.5429 | 0.0367 | 3 | 0 |
| WCM155 | 11 | 193096 | 134605954 | −0.1116 | 0.486 | 1.7109 | 1.0902 | 2 | 1 |
| WCM155 | 12 | 148430 | 16757863 | −0.4379 | 0.0698 | 2.0549 | 0.0442 | 2 | 0 |
| WCM155 | 12 | 18234316 | 22843284 | 0.5247 | 1.2399 | 2.5019 | 2.2216 | 3 | 2 |
| WCM155 | 12 | 22843569 | 34180172 | −0.4867 | 0.0051 | 1.9327 | 0.0744 | 2 | 0 |
| WCM155 | 12 | 34180370 | 104032032 | 0.3364 | 1.0228 | 2.0319 | 2.0319 | 2 | 2 |
| WCM155 | 12 | 104034050 | 133810880 | −0.3724 | 0.1555 | 2.2049 | 0.0227 | 2 | 0 |
| WCM155 | 13 | 19041937 | 47315970 | −0.0569 | 0.5534 | 2.9083 | 0.0268 | 3 | 0 |
| WCM155 | 13 | 47317388 | 47367537 | −2.0257 | −2.9802 | NA | NA | NA | NA |
| WCM155 | 13 | 47396144 | 115089411 | −0.0504 | 0.5614 | 2.9049 | 0.0466 | 3 | 0 |
| WCM155 | 14 | 19110239 | 107283270 | 0.6165 | 1.3441 | 3.1994 | 1.8782 | 3 | 2 |
| WCM155 | 15 | 20083968 | 22933511 | −0.4535 | 0.0492 | 1.6776 | 0.3917 | 2 | 0 |
| WCM155 | 15 | 22933760 | 102514218 | −0.4674 | 0.0308 | 1.9972 | 0.0461 | 2 | 0 |
| WCM155 | 16 | 97584 | 10274308 | −0.0506 | 0.5612 | 1.8065 | 1.1444 | 2 | 1 |
| WCM155 | 16 | 10275755 | 11876217 | −1.1254 | −0.9287 | 1.0096 | 0.0411 | 1 | 0 |
| WCM155 | 16 | 11913796 | 30129735 | −0.1537 | 0.4337 | 1.6555 | 1.0459 | 2 | 1 |
| WCM155 | 16 | 30133298 | 31044683 | −1.2245 | −1.0944 | 0.959 | −0.0223 | 1 | 0 |
| WCM155 | 16 | 31045168 | 48204077 | −0.1076 | 0.4909 | 1.5983 | 1.2123 | 2 | 1 |
| WCM155 | 16 | 48204832 | 57017527 | −0.5169 | −0.0352 | 1.9185 | 0.0332 | 2 | 0 |
| WCM155 | 16 | 57051133 | 74446830 | −0.0789 | 0.5264 | 1.6833 | 1.1973 | 2 | 1 |
| WCM155 | 16 | 74447064 | 90260461 | −0.4877 | 0.0038 | 1.9739 | 0.0314 | 2 | 0 |
| WCM155 | 17 | 131595 | 27577326 | 0.1653 | 0.8216 | 1.7674 | 1.7674 | 2 | 2 |
| WCM155 | 17 | 27579045 | 40975970 | −0.5066 | −0.0215 | 1.9161 | 0.0543 | 2 | 0 |
| WCM155 | 17 | 40976245 | 81187854 | 0.2546 | 0.9272 | 1.9015 | 1.9015 | 2 | 2 |
| WCM155 | 18 | 47711 | 50683793 | −0.0506 | 0.5612 | 1.7179 | 1.2331 | 2 | 1 |
| WCM155 | 18 | 50705361 | 78005226 | −0.9699 | −0.6825 | 1.2381 | 0.0081 | 1 | 0 |
| WCM155 | 19 | 105050 | 40196683 | −0.0482 | 0.5641 | 1.5111 | 1.4459 | 2 | 1 |
| WCM155 | 19 | 40197328 | 42713835 | 0.2774 | 0.9539 | 2.6409 | 1.2333 | 3 | 1 |
| WCM155 | 19 | 42719324 | 42806483 | −0.8862 | −0.556 | NA | NA | NA | NA |
| WCM155 | 19 | 42813884 | 46444066 | 0.0109 | 0.6362 | 1.5845 | 1.5241 | 2 | 2 |
| WCM155 | 19 | 46457052 | 47217284 | −0.6034 | −0.1524 | 1.8097 | −0.0103 | 2 | 0 |
| WCM155 | 19 | 47219499 | 59093645 | 0.0655 | 0.7022 | 1.627 | 1.627 | 2 | 2 |
| WCM155 | 2 | 41615 | 14777883 | −0.5084 | −0.0238 | 0.9836 | 0.9836 | 1 | 1 |
| WCM155 | 2 | 14778142 | 187498107 | −0.2421 | 0.3226 | 1.2506 | 1.2506 | 1 | 1 |
| WCM155 | 2 | 187500767 | 243036958 | −0.4498 | 0.0541 | 1.0382 | 1.0382 | 1 | 1 |
| WCM155 | 20 | 68311 | 62934623 | 0.1446 | 0.7971 | 1.7376 | 1.7376 | 2 | 2 |
| WCM155 | 21 | 9590370 | 48084114 | 0.4988 | 1.2102 | 2.7954 | 1.8319 | 3 | 2 |
| WCM155 | 22 | 16159044 | 46486943 | −0.4567 | 0.045 | 2.0144 | 0.0489 | 2 | 0 |
| WCM155 | 22 | 46493741 | 51223730 | 0.2871 | 0.9653 | 1.9525 | 1.9525 | 2 | 2 |
| WCM155 | 3 | 239451 | 9547806 | 0.1659 | 0.8224 | 1.7683 | 1.7683 | 2 | 2 |
| WCM155 | 3 | 9594037 | 11888165 | −0.4936 | −0.004 | 1.9845 | 0.0099 | 2 | 0 |
| WCM155 | 3 | 11902005 | 70017107 | 0.2231 | 0.8901 | 1.8534 | 1.8534 | 2 | 2 |
| WCM155 | 3 | 70017459 | 72311601 | −0.7385 | −0.341 | NA | NA | NA | NA |
| WCM155 | 3 | 72423904 | 197896800 | 0.1981 | 0.8605 | 1.8157 | 1.8157 | 2 | 2 |
| WCM155 | 4 | 53336 | 17963681 | −0.476 | 0.0194 | 1.0135 | 1.0135 | 1 | 1 |
| WCM155 | 4 | 17964594 | 42965095 | −1.1945 | −1.0434 | 0.9599 | 0.0104 | 1 | 0 |
| WCM155 | 4 | 43022362 | 146086293 | −0.5093 | −0.025 | 0.9828 | 0.9828 | 1 | 1 |
| WCM155 | 4 | 146092923 | 147863881 | −1.1618 | −0.9886 | NA | NA | NA | NA |
| WCM155 | 4 | 148265864 | 190903996 | −0.5272 | −0.049 | 0.9666 | 0.9666 | 1 | 1 |
| WCM155 | 5 | 90537 | 180687847 | −0.111 | 0.4867 | 1.7169 | 1.0855 | 2 | 1 |
| WCM155 | 6 | 203588 | 73879528 | 0.1941 | 0.8558 | 2.6414 | 0.9783 | 3 | 1 |
| WCM155 | 6 | 73900433 | 117249987 | −0.1639 | 0.4209 | 2.6564 | 0.0212 | 3 | 0 |
| WCM155 | 6 | 117250139 | 170892860 | 0.1441 | 0.7964 | 2.5234 | 0.9501 | 3 | 1 |
| WCM155 | 7 | 193254 | 141628158 | −0.109 | 0.4892 | 1.7198 | 1.0875 | 2 | 1 |
| WCM155 | 7 | 141629933 | 158937046 | 0.4981 | 1.2094 | 2.6643 | 1.9605 | 3 | 2 |
| WCM155 | 8 | 163470 | 7631260 | −0.5384 | −0.0641 | 1.8801 | 0.033 | 2 | 0 |
| WCM155 | 8 | 7717895 | 8086107 | −2.3362 | −4.7314 | NA | NA | NA | NA |
| WCM155 | 8 | 8090350 | 42698270 | −0.5063 | −0.0211 | 1.9351 | 0.0359 | 2 | 0 |
| WCM155 | 8 | 42705973 | 146279528 | 0.4174 | 1.1168 | 2.1686 | 2.1686 | 2 | 2 |
| WCM155 | 9 | 14908 | 21512219 | −0.5111 | −0.0274 | 1.9066 | 0.0558 | 2 | 0 |
| WCM155 | 9 | 21802811 | 23815002 | −2.3709 | −5.1165 | NA | NA | NA | NA |
| WCM155 | 9 | 24543350 | 27567137 | −0.605 | −0.1547 | 1.8319 | −0.0353 | 2 | 0 |
| WCM155 | 9 | 27610103 | 141107536 | 0.2373 | 0.9068 | 1.8749 | 1.8749 | 2 | 2 |
| WCM155 | X | 2700084 | 154493598 | 0.1189 | 0.7664 | NA | NA | NA | NA |
| WCM155_Z1_1 | 1 | 69634 | 22336336 | −0.6878 | −0.0339 | 1.0463 | 0.9073 | 1 | 1 |
| WCM155_Z1_1 | 1 | 22405202 | 22834595 | −1.5575 | −0.9418 | NA | NA | NA | NA |
| WCM155_Z1_1 | 1 | 22835077 | 22987537 | −0.6586 | −0.0037 | NA | NA | NA | NA |
| WCM155_Z1_1 | 1 | 22987618 | 26131654 | −1.6325 | −1.0214 | 0.9958 | −0.0105 | 1 | 0 |
| WCM155_Z1_1 | 1 | 26135156 | 29863127 | −0.1748 | 0.4928 | 1.8228 | 0.9916 | 2 | 1 |
| WCM155_Z1_1 | 1 | 31186459 | 154033493 | 0.2943 | 0.9707 | 2.9252 | 0.9943 | 3 | 1 |
| WCM155_Z1_1 | 1 | 154042912 | 154293675 | 1.0722 | 1.758 | NA | NA | NA | NA |
| WCM155_Z1_1 | 1 | 154294529 | 249212401 | 0.2758 | 0.9518 | 2.9268 | 0.9419 | 3 | 1 |
| WCM155_Z1_1 | 10 | 225979 | 1599093 | 0.576 | 1.2563 | NA | NA | NA | NA |
| WCM155_Z1_1 | 10 | 1779275 | 61552524 | −0.2249 | 0.4416 | 2.7193 | −0.0031 | 3 | 0 |
| WCM155_Z1_1 | 10 | 61552637 | 88730500 | 0.2283 | 0.9036 | 2.8235 | 0.918 | 3 | 1 |
| WCM155_Z1_1 | 10 | 88764111 | 135440068 | −0.1864 | 0.4809 | 2.7937 | −0.0024 | 3 | 0 |
| WCM155_Z1_1 | 11 | 193112 | 134856626 | −0.1485 | 0.5196 | 1.8934 | 0.9737 | 2 | 1 |
| WCM155_Z1_1 | 12 | 148644 | 17826233 | −0.4963 | 0.1634 | 2.2477 | −0.0079 | 2 | 0 |
| WCM155_Z1_1 | 12 | 18234288 | 22837880 | 0.5777 | 1.2581 | 2.4354 | 2.3482 | 2 | 2 |
| WCM155_Z1_1 | 12 | 23686050 | 34182560 | −0.5584 | 0.0995 | 2.1517 | −0.0089 | 2 | 0 |
| WCM155_Z1_1 | 12 | 38710847 | 49920023 | 0.3228 | 0.9996 | 2.0868 | 1.9121 | 2 | 2 |
| WCM155_Z1_1 | 12 | 49933229 | 80837600 | 0.4244 | 1.1028 | 2.1477 | 2.1477 | 2 | 2 |
| WCM155_Z1_1 | 12 | 80838125 | 104034014 | 0.3651 | 1.0425 | 2.1581 | 1.9615 | 2 | 2 |
| WCM155_Z1_1 | 12 | 104042576 | 133810926 | −0.574 | 0.0834 | 2.1173 | 0.0018 | 2 | 0 |
| WCM155_Z1_1 | 13 | 19600159 | 115089411 | −0.0979 | 0.5713 | 2.9724 | −6.00E−04 | 3 | 0 |
| WCM155_Z1_1 | 14 | 19377727 | 107283047 | 0.7687 | 1.4514 | 3.7719 | 1.6976 | 4 | 2 |
| WCM155_Z1_1 | 15 | 20170150 | 102517642 | −0.6963 | −0.0426 | 1.9427 | −9.00E−04 | 2 | 0 |
| WCM155_Z1_1 | 16 | 97486 | 10274203 | −0.1249 | 0.5438 | 1.9223 | 0.9933 | 2 | 1 |
| WCM155_Z1_1 | 16 | 10524563 | 11914071 | −1.6181 | −1.0061 | 1.0098 | −0.014 | 1 | 0 |
| WCM155_Z1_1 | 16 | 11931840 | 30194351 | −0.1577 | 0.5102 | 1.8594 | 0.9892 | 2 | 1 |
| WCM155_Z1_1 | 16 | 30196714 | 31012907 | −1.6152 | −1.003 | 0.9928 | 0.0051 | 1 | 0 |
| WCM155_Z1_1 | 16 | 31045389 | 48204026 | −0.1533 | 0.5147 | 1.8681 | 0.9894 | 2 | 1 |
| WCM155_Z1_1 | 16 | 48204820 | 57054762 | −0.6796 | −0.0254 | 1.9758 | −0.0107 | 2 | 0 |
| WCM155_Z1_1 | 16 | 57056170 | 74366531 | −0.0484 | 0.6219 | 2.0616 | 1.0161 | 2 | 1 |
| WCM155_Z1_1 | 16 | 74371376 | 90233622 | −0.5347 | 0.1238 | 2.1765 | 0.0027 | 2 | 0 |
| WCM155_Z1_1 | 17 | 131595 | 27493713 | 0.1593 | 0.8334 | 1.8037 | 1.7602 | 2 | 2 |
| WCM155_Z1_1 | 17 | 27493802 | 40972815 | −0.6837 | −0.0296 | 1.959 | 4.00E−04 | 2 | 0 |
| WCM155_Z1_1 | 17 | 40975773 | 81083561 | 0.3846 | 1.0624 | 2.1138 | 2.0629 | 2 | 2 |
| WCM155_Z1_1 | 18 | 48202 | 50592427 | −0.0965 | 0.5728 | 1.9155 | 1.0592 | 2 | 1 |
| WCM155_Z1_1 | 18 | 50683803 | 64235922 | −1.5413 | −0.9247 | 1.0689 | −0.0153 | 1 | 0 |
| WCM155_Z1_1 | 18 | 64239283 | 66678298 | −0.1658 | 0.5019 | NA | NA | NA | NA |
| WCM155_Z1_1 | 18 | 67068541 | 70532137 | −1.5374 | −0.9205 | NA | NA | NA | NA |
| WCM155_Z1_1 | 18 | 70532441 | 78005206 | −0.8013 | −0.151 | 1.8023 | −0.0011 | 2 | 0 |
| WCM155_Z1_1 | 19 | 281415 | 40276696 | −0.1425 | 0.5258 | 1.9147 | 0.9647 | 2 | 1 |
| WCM155_Z1_1 | 19 | 40316358 | 42626711 | 0.2755 | 0.9516 | 2.9105 | 0.9574 | 3 | 1 |
| WCM155_Z1_1 | 19 | 42636543 | 42806417 | −1.6452 | −1.0348 | NA | NA | NA | NA |
| WCM155_Z1_1 | 19 | 42813955 | 46417907 | −0.1479 | 0.5202 | 1.8942 | 0.9741 | 2 | 1 |
| WCM155_Z1_1 | 19 | 46443397 | 47219580 | −0.7699 | −0.1186 | 1.8502 | −0.0081 | 2 | 0 |
| WCM155_Z1_1 | 19 | 47223949 | 59083988 | 0.2182 | 0.8933 | 1.882 | 1.8329 | 2 | 2 |
| WCM155_Z1_1 | 2 | 41620 | 15307391 | −0.6807 | −0.0265 | 1.0082 | 0.9555 | 1 | 1 |
| WCM155_Z1_1 | 2 | 15307442 | 186696610 | −0.1898 | 0.4775 | 1.825 | 0.9597 | 2 | 1 |
| WCM155_Z1_1 | 2 | 186697938 | 201541625 | −0.7019 | −0.0484 | 1.0828 | 0.8513 | 1 | 1 |
| WCM155_Z1_1 | 2 | 201677131 | 201760020 | −1.6823 | −1.0744 | NA | NA | NA | NA |
| WCM155_Z1_1 | 2 | 201761859 | 242946616 | −0.7024 | −0.0489 | 1.0584 | 0.8749 | 1 | 1 |
| WCM155_Z1_1 | 20 | 68406 | 62904845 | 0.2413 | 0.9168 | 1.8879 | 1.8879 | 2 | 2 |
| WCM155_Z1_1 | 21 | 9826235 | 48084282 | 0.6209 | 1.3018 | 3.0745 | 1.8563 | 3 | 2 |
| WCM155_Z1_1 | 22 | 17063476 | 46449629 | −0.6842 | −0.0302 | 1.9592 | −6.00E−04 | 2 | 0 |
| WCM155_Z1_1 | 22 | 46449924 | 51219138 | 0.1809 | 0.8554 | 1.8534 | 1.7651 | 2 | 2 |
| WCM155_Z1_1 | 3 | 239431 | 9543958 | 0.3152 | 0.9919 | 1.9968 | 1.9807 | 2 | 2 |
| WCM155_Z1_1 | 3 | 9547696 | 11887993 | −0.5619 | 0.0959 | 2.1464 | −0.0089 | 2 | 0 |
| WCM155_Z1_1 | 3 | 12046076 | 69998309 | 0.3142 | 0.9908 | 2.0095 | 1.9651 | 2 | 2 |
| WCM155_Z1_1 | 3 | 70000995 | 71834110 | −0.6288 | 0.027 | NA | NA | NA | NA |
| WCM155_Z1_1 | 3 | 72427727 | 197894712 | 0.2971 | 0.9734 | 1.9755 | 1.9516 | 2 | 2 |
| WCM155_Z1_1 | 4 | 59334 | 17887713 | −0.6876 | −0.0336 | 1.0506 | 0.9034 | 1 | 1 |
| WCM155_Z1_1 | 4 | 17910840 | 43032460 | −1.6182 | −1.0063 | 0.9986 | −0.0029 | 1 | 0 |
| WCM155_Z1_1 | 4 | 44176927 | 146475191 | −0.7165 | −0.0635 | 0.9797 | 0.9342 | 1 | 1 |
| WCM155_Z1_1 | 4 | 146478954 | 148265854 | −1.5998 | −0.9867 | NA | NA | NA | NA |
| WCM155_Z1_1 | 4 | 148406880 | 190426471 | −0.7164 | −0.0633 | 0.9847 | 0.9294 | 1 | 1 |
| WCM155_Z1_1 | 5 | 140480 | 180687800 | −0.1883 | 0.479 | 1.8312 | 0.9564 | 2 | 1 |
| WCM155_Z1_1 | 6 | 203583 | 73904476 | 0.2396 | 0.9151 | 2.8651 | 0.9063 | 3 | 1 |
| WCM155_Z1_1 | 6 | 73904851 | 117589385 | −0.178 | 0.4895 | 2.8101 | −0.0022 | 3 | 0 |
| WCM155_Z1_1 | 6 | 117589621 | 170893415 | 0.2196 | 0.8947 | 2.7855 | 0.9331 | 3 | 1 |
| WCM155_Z1_1 | 7 | 193254 | 141619380 | −0.1434 | 0.5248 | 1.8988 | 0.9787 | 2 | 1 |
| WCM155_Z1_1 | 7 | 141629934 | 158937400 | 0.5833 | 1.2638 | 2.9387 | 1.8637 | 3 | 2 |
| WCM155_Z1_1 | 8 | 190907 | 42698213 | −0.8305 | −0.1813 | 1.7674 | −0.0035 | 2 | 0 |
| WCM155_Z1_1 | 8 | 42705962 | 146279545 | 0.2674 | 0.9433 | 2.0969 | 1.7491 | 2 | 2 |
| WCM155_Z1_1 | 9 | 17079 | 27949279 | −0.7122 | −0.0591 | 1.9309 | −0.0111 | 2 | 0 |
| WCM155_Z1_1 | 9 | 27949467 | 141111414 | 0.2328 | 0.9082 | 1.8767 | 1.8767 | 2 | 2 |
| WCM155_Z1_1 | X | 2700159 | 154509348 | −0.0109 | 0.6601 | NA | NA | NA | NA |
| WCM155_Z2_1 | 1 | 69610 | 22987834 | −0.5832 | −0.0243 | 1.0793 | 0.8873 | 1 | 1 |
| WCM155_Z2_1 | 1 | 23046058 | 26131654 | −1.0756 | −0.7836 | 1.124 | 0.0377 | 1 | 0 |
| WCM155_Z2_1 | 1 | 26135075 | 29475580 | −0.1314 | 0.5803 | 1.9044 | 1.0859 | 2 | 1 |
| WCM155_Z2_1 | 1 | 29476670 | 249212388 | 0.2268 | 1.0227 | 3.0907 | 0.9727 | 3 | 1 |
| WCM155_Z2_1 | 10 | 225979 | 3143115 | 0.4606 | 1.2992 | NA | NA | NA | NA |
| WCM155_Z2_1 | 10 | 3143624 | 57359707 | −0.1131 | 0.6035 | 3.0386 | 3.00E−04 | 3 | 0 |
| WCM155_Z2_1 | 10 | 58117933 | 60128497 | −1.8535 | −2.5689 | NA | NA | NA | NA |
| WCM155_Z2_1 | 10 | 60145356 | 61552397 | −0.2131 | 0.4755 | NA | NA | NA | NA |
| WCM155_Z2_1 | 10 | 61552694 | 88811601 | 0.2616 | 1.0644 | 3.0786 | 1.1039 | 3 | 1 |
| WCM155_Z2_1 | 10 | 88813150 | 135440117 | −0.1151 | 0.601 | 3.0338 | −3.00E−04 | 3 | 0 |
| WCM155_Z2_1 | 11 | 193112 | 61036462 | −0.1146 | 0.6016 | 2.0239 | 1.0109 | 2 | 1 |
| WCM155_Z2_1 | 11 | 61039162 | 85576181 | −0.0771 | 0.649 | 2.1176 | 1.0186 | 2 | 1 |
| WCM155_Z2_1 | 11 | 85584303 | 134856626 | −0.1335 | 0.5776 | 2.0018 | 0.9829 | 2 | 1 |
| WCM155_Z2_1 | 12 | 148637 | 17826293 | −0.4945 | 0.0997 | 2.106 | 0.037 | 2 | 0 |
| WCM155_Z2_1 | 12 | 18234236 | 22837885 | 0.2213 | 1.016 | 2.0831 | 1.9615 | 2 | 2 |
| WCM155_Z2_1 | 12 | 23686046 | 34182560 | −0.579 | −0.0183 | 1.9993 | −0.0246 | 2 | 0 |
| WCM155_Z2_1 | 12 | 38710800 | 104033980 | 0.2318 | 1.0286 | 2.0499 | 2.0303 | 2 | 2 |
| WCM155_Z2_1 | 12 | 104042484 | 133810794 | −0.5525 | 0.019 | 2.0241 | 0.0024 | 2 | 0 |
| WCM155_Z2_1 | 13 | 19600147 | 115089411 | −0.0755 | 0.6511 | 3.1435 | −0.0028 | 3 | 0 |
| WCM155_Z2_1 | 14 | 19377733 | 107283060 | 0.4341 | 1.2682 | 2.9213 | 1.8959 | 3 | 2 |
| WCM155_Z2_1 | 15 | 20170150 | 102517639 | −0.5598 | 0.0087 | 2.0061 | 0.006 | 2 | 0 |
| WCM155_Z2_1 | 16 | 97464 | 10775855 | −0.1843 | 0.5126 | 1.8927 | 0.9605 | 2 | 1 |
| WCM155_Z2_1 | 16 | 10783119 | 11868148 | −1.2348 | −1.0665 | NA | NA | NA | NA |
| WCM155_Z2_1 | 16 | 11868856 | 30199807 | −0.5731 | −0.01 | 1.0113 | 0.9749 | 1 | 1 |
| WCM155_Z2_1 | 16 | 30217373 | 31051114 | −1.096 | −0.8186 | 1.0664 | 0.0676 | 1 | 0 |
| WCM155_Z2_1 | 16 | 31072510 | 48177864 | −0.1824 | 0.515 | 1.8975 | 0.9604 | 2 | 1 |
| WCM155_Z2_1 | 16 | 48180277 | 57054692 | −0.5821 | −0.0227 | 1.9956 | −0.0268 | 2 | 0 |
| WCM155_Z2_1 | 16 | 57056167 | 74366531 | −0.1414 | 0.5675 | 1.9981 | 0.9659 | 2 | 1 |
| WCM155_Z2_1 | 16 | 74371512 | 90233622 | −0.6065 | −0.0574 | 1.9484 | −0.0264 | 2 | 0 |
| WCM155_Z2_1 | 17 | 131595 | 27493920 | 0.0964 | 0.8645 | 1.8386 | 1.8029 | 2 | 2 |
| WCM155_Z2_1 | 17 | 27573886 | 40972834 | −0.5558 | 0.0143 | 2.0284 | −0.0085 | 2 | 0 |
| WCM155_Z2_1 | 17 | 40975796 | 81083561 | 0.2017 | 0.9924 | 1.9895 | 1.9895 | 2 | 2 |
| WCM155_Z2_1 | 18 | 48202 | 50592425 | −0.126 | 0.5872 | 1.9532 | 1.0514 | 2 | 1 |
| WCM155_Z2_1 | 18 | 50683882 | 71803073 | −1.0917 | −0.8113 | 1.1167 | 0.023 | 1 | 0 |
| WCM155_Z2_1 | 18 | 71807493 | 78005164 | −0.5944 | −0.0402 | 1.8715 | 0.0735 | 2 | 0 |
| WCM155_Z2_1 | 19 | 281535 | 40317363 | −0.1902 | 0.505 | 1.8668 | 0.9713 | 2 | 1 |
| WCM155_Z2_1 | 19 | 40318021 | 42621152 | 0.2477 | 1.0477 | 3.1739 | 0.9606 | 3 | 1 |
| WCM155_Z2_1 | 19 | 42621404 | 42806423 | −0.9569 | −0.5871 | NA | NA | NA | NA |
| WCM155_Z2_1 | 19 | 42813797 | 46444058 | −0.1707 | 0.5301 | 1.8627 | 1.0254 | 2 | 1 |
| WCM155_Z2_1 | 19 | 46444177 | 47242116 | −0.6074 | −0.0587 | 1.9934 | −0.0731 | 2 | 0 |
| WCM155_Z2_1 | 19 | 47258832 | 59083988 | 0.1133 | 0.8852 | 1.8628 | 1.8313 | 2 | 2 |
| WCM155_Z2_1 | 2 | 41614 | 11800179 | −0.6001 | −0.0482 | 1.0015 | 0.9327 | 1 | 1 |
| WCM155_Z2_1 | 2 | 11802135 | 186666103 | −0.1853 | 0.5112 | 1.8562 | 0.9944 | 2 | 1 |
| WCM155_Z2_1 | 2 | 186666414 | 242946607 | −0.5885 | −0.0317 | 0.9935 | 0.963 | 1 | 1 |
| WCM155_Z2_1 | 20 | 68397 | 62904881 | −0.1209 | 0.5936 | 1.9787 | 1.0392 | 2 | 1 |
| WCM155_Z2_1 | 21 | 9826230 | 48084214 | 0.1357 | 0.9124 | 1.896 | 1.8685 | 2 | 2 |
| WCM155_Z2_1 | 22 | 17063431 | 46494384 | −0.566 | −1.00E−04 | 2.021 | −0.0211 | 2 | 0 |
| WCM155_Z2_1 | 22 | 46501443 | 51219138 | 0.1487 | 0.9283 | 1.9678 | 1.8384 | 2 | 2 |
| WCM155_Z2_1 | 3 | 239451 | 9547837 | −0.1549 | 0.5503 | 1.9719 | 0.9569 | 2 | 1 |
| WCM155_Z2_1 | 3 | 9594120 | 12278890 | −0.5265 | 0.0554 | 1.9511 | 0.1271 | 2 | 0 |
| WCM155_Z2_1 | 3 | 12393092 | 197894712 | −0.1461 | 0.5616 | 1.9978 | 0.9539 | 2 | 1 |
| WCM155_Z2_1 | 4 | 59390 | 20552510 | −0.556 | 0.014 | 1.0282 | 0.9913 | 1 | 1 |
| WCM155_Z2_1 | 4 | 20555479 | 42658879 | −1.205 | −1.0116 | 0.9799 | 0.0121 | 1 | 0 |
| WCM155_Z2_1 | 4 | 42895389 | 190426471 | −0.5324 | 0.0471 | 1.0606 | 1.0058 | 1 | 1 |
| WCM155_Z2_1 | 5 | 140443 | 180687800 | −0.1187 | 0.5964 | 2.0363 | 0.9877 | 2 | 1 |
| WCM155_Z2_1 | 6 | 203588 | 73787085 | 0.194 | 0.9831 | 2.9666 | 0.9869 | 3 | 1 |
| WCM155_Z2_1 | 6 | 73787517 | 117589463 | −0.1168 | 0.5988 | 3.0393 | −0.0104 | 3 | 0 |
| WCM155_Z2_1 | 6 | 117589612 | 170893427 | 0.2115 | 1.0042 | 2.9908 | 1.0208 | 3 | 1 |
| WCM155_Z2_1 | 7 | 193245 | 141635637 | −0.1143 | 0.6021 | 1.9744 | 1.0613 | 2 | 1 |
| WCM155_Z2_1 | 7 | 141643719 | 158937400 | 0.4544 | 1.2919 | 2.9686 | 1.9286 | 3 | 2 |
| WCM155_Z2_1 | 8 | 190900 | 42693429 | −0.5512 | 0.0208 | 2.0149 | 0.0142 | 2 | 0 |
| WCM155_Z2_1 | 8 | 42694404 | 146279507 | 0.1917 | 0.9804 | 1.9849 | 1.9611 | 2 | 2 |
| WCM155_Z2_1 | 9 | 17053 | 27949026 | −0.5349 | 0.0436 | 2.0236 | 0.0377 | 2 | 0 |
| WCM155_Z2_1 | 9 | 27949189 | 141111414 | 0.1724 | 0.957 | 1.9413 | 1.9413 | 2 | 2 |
| WCM155_Z2_1 | X | 2700147 | 154509348 | 0.2048 | 0.9962 | NA | NA | NA | NA |
| WCM156 | 1 | 14896 | 117120081 | −0.008 | 0.0593 | 1.042 | 1.042 | 1 | 1 |
| WCM156 | 1 | 117122058 | 160784336 | 0.1167 | 0.9622 | 1.9483 | 1.9483 | 2 | 2 |
| WCM156 | 1 | 160786632 | 197404692 | −0.016 | −0.0196 | 0.9865 | 0.9865 | 1 | 1 |
| WCM156 | 1 | 197407787 | 248512064 | 0.1049 | 0.8945 | 1.8589 | 1.8589 | 2 | 2 |
| WCM156 | 1 | 248512498 | 249213961 | 0.0101 | 0.2244 | 2.5004 | −0.1637 | 3 | 0 |
| WCM156 | 10 | 225938 | 135439032 | −0.0413 | −0.3 | 0.8122 | 0.8122 | 1 | 1 |
| WCM156 | 11 | 193096 | 134605906 | −0.0123 | 0.0173 | 1.0121 | 1.0121 | 1 | 1 |
| WCM156 | 12 | 148452 | 133810880 | −0.0232 | −0.0948 | 0.9364 | 0.9364 | 1 | 1 |
| WCM156 | 13 | 19042005 | 29012330 | −0.0107 | 0.033 | 1.0232 | 1.0232 | 1 | 1 |
| WCM156 | 13 | 29040947 | 74268850 | −0.1602 | −4.7778 | 0.0365 | 0.0365 | 0 | 0 |
| WCM156 | 13 | 74269160 | 115089411 | 0.0715 | 0.6874 | 1.6104 | 1.6104 | 2 | 2 |
| WCM156 | 14 | 19110239 | 107283263 | −0.1003 | −1.2536 | 0.4194 | 0.4194 | 0 | 0 |
| WCM156 | 15 | 20083896 | 35664328 | 0.0197 | 0.3059 | 1.2361 | 1.2361 | 1 | 1 |
| WCM156 | 15 | 35664559 | 53081788 | −0.1384 | −2.5234 | 0.1739 | 0.1739 | 0 | 0 |
| WCM156 | 15 | 53176757 | 102515241 | −0.0018 | 0.118 | 1.0852 | 1.0852 | 1 | 1 |
| WCM156 | 16 | 64430 | 90168697 | −0.0405 | −0.2899 | 0.818 | 0.818 | 1 | 1 |
| WCM156 | 17 | 131595 | 8445626 | −0.166 | −11.7873 | 3.00E−04 | 3.00E−04 | 0 | 0 |
| WCM156 | 17 | 8448822 | 81052545 | 0.0206 | 0.3132 | 1.2425 | 1.2425 | 1 | 1 |
| WCM156 | 18 | 47682 | 78005145 | −0.0406 | −0.2914 | 0.8171 | 0.8171 | 1 | 1 |
| WCM156 | 19 | 105050 | 59093645 | −0.0067 | 0.0711 | 1.0505 | 1.0505 | 1 | 1 |
| WCM156 | 2 | 41613 | 243037091 | 0.007 | 0.1977 | 1.1468 | 1.1468 | 1 | 1 |
| WCM156 | 20 | 68312 | 62934623 | −1.00E−04 | 0.1332 | 1.0967 | 1.0967 | 1 | 1 |
| WCM156 | 21 | 9590311 | 48084210 | −0.0459 | −0.3564 | 0.7811 | 0.7811 | 1 | 1 |
| WCM156 | 22 | 16159043 | 51219090 | 0.0104 | 0.2269 | 1.1703 | 1.1703 | 1 | 1 |
| WCM156 | 3 | 239459 | 113505143 | −0.0402 | −0.2858 | 0.8203 | 0.8203 | 1 | 1 |
| WCM156 | 3 | 113505277 | 173999810 | 0.078 | 0.7297 | 1.6583 | 1.6583 | 2 | 2 |
| WCM156 | 3 | 174000019 | 197896708 | −0.0262 | −0.1271 | 0.9157 | 0.9157 | 1 | 1 |
| WCM156 | 4 | 53361 | 190878508 | −0.0301 | −0.1697 | 0.889 | 0.889 | 1 | 1 |
| WCM156 | 5 | 90680 | 180687854 | −0.0398 | −0.2815 | 0.8227 | 0.8227 | 1 | 1 |
| WCM156 | 6 | 203475 | 76388512 | 0.0313 | 0.3998 | 1.3193 | 1.3193 | 1 | 1 |
| WCM156 | 6 | 76388561 | 170892712 | −0.1023 | −1.2997 | 0.4062 | 0.4062 | 0 | 0 |
| WCM156 | 7 | 193205 | 158937135 | −0.0241 | −0.1044 | 0.9302 | 0.9302 | 1 | 1 |
| WCM156 | 8 | 163438 | 68110923 | −0.0783 | −0.8265 | 0.5639 | 0.5639 | 1 | 1 |
| WCM156 | 8 | 68111147 | 146279396 | 0.093 | 0.8239 | 1.7702 | 1.7702 | 2 | 2 |
| WCM156 | 9 | 116808 | 141107525 | 0.0353 | 0.4306 | 1.3478 | 1.3478 | 1 | 1 |
| WCM156 | X | 2700162 | 154493589 | −0.1808 | NA | NA | NA | NA | NA |
| WCM156_Z5_1 | 1 | 721439 | 878434 | −0.1011 | −0.1138 | NA | NA | NA | NA |
| WCM156_Z5_1 | 1 | 878734 | 11276286 | −0.4587 | −0.9475 | 1.0288 | 0.0082 | 1 | 0 |
| WCM156_Z5_1 | 1 | 11288758 | 93302926 | −0.0403 | 0.0052 | 1.0171 | 0.9901 | 1 | 1 |
| WCM156_Z5_1 | 1 | 93303154 | 95360379 | −0.4945 | −1.0493 | NA | NA | NA | NA |
| WCM156_Z5_1 | 1 | 95363337 | 145699074 | −0.0583 | −0.0296 | 1.0196 | 0.9397 | 1 | 1 |
| WCM156_Z5_1 | 1 | 145704016 | 160771581 | 0.3051 | 0.6086 | 1.9727 | 1.0768 | 2 | 1 |
| WCM156_Z5_1 | 1 | 160783679 | 190250846 | −0.0741 | −0.0605 | 1.994 | −0.0762 | 2 | 0 |
| WCM156_Z5_1 | 1 | 190423929 | 249212346 | 0.2803 | 0.5685 | 2.9796 | −0.0136 | 3 | 0 |
| WCM156_Z5_1 | 10 | 225980 | 24762793 | −0.4892 | −1.034 | 1.0798 | −0.103 | 1 | 0 |
| WCM156_Z5_1 | 10 | 24762971 | 87675947 | −0.0428 | 3.00E−04 | 1.0502 | 0.9502 | 1 | 1 |
| WCM156_Z5_1 | 10 | 87898734 | 96234541 | −0.4745 | −0.9919 | 1.0162 | −0.0105 | 1 | 0 |
| WCM156_Z5_1 | 10 | 96253145 | 104934633 | −0.0545 | −0.0222 | 1.0115 | 0.958 | 1 | 1 |
| WCM156_Z5_1 | 10 | 105005970 | 105386870 | 0.3345 | 0.6554 | NA | NA | NA | NA |
| WCM156_Z5_1 | 10 | 105420796 | 129923888 | −0.0432 | −3.00E−04 | 0.9998 | 0.9998 | 1 | 1 |
| WCM156_Z5_1 | 10 | 131265549 | 135440080 | −0.4215 | −0.8464 | 1.0843 | 0.0281 | 1 | 0 |
| WCM156_Z5_1 | 11 | 193127 | 134856597 | −0.0386 | 0.0085 | 1.0233 | 0.9885 | 1 | 1 |
| WCM156_Z5_1 | 12 | 148639 | 68042938 | −0.0548 | −0.0228 | 1.0053 | 0.9634 | 1 | 1 |
| WCM156_Z5_1 | 12 | 68043592 | 80602001 | 1.0459 | 1.6633 | 5.3723 | 0.9627 | 5 | 1 |
| WCM156_Z5_1 | 12 | 80603289 | 133810704 | 0.2831 | 0.5731 | 3.0138 | −0.0383 | 3 | 0 |
| WCM156_Z5_1 | 13 | 19600159 | 31543095 | −0.0863 | −0.0845 | 0.9879 | 0.8983 | 1 | 1 |
| WCM156_Z5_1 | 13 | 31549017 | 31903679 | −0.3719 | −0.718 | NA | NA | NA | NA |
| WCM156_Z5_1 | 13 | 32313822 | 33226096 | −1.1235 | NA | NA | NA | NA | NA |
| WCM156_Z5_1 | 13 | 33226199 | 73649950 | −0.4022 | −0.7956 | 1.2357 | −0.0835 | 1 | 0 |
| WCM156_Z5_1 | 13 | 74269778 | 88330464 | 0.0682 | 0.2067 | 1.26 | 1.0481 | 1 | 1 |
| WCM156_Z5_1 | 13 | 90883524 | 115039326 | 0.3559 | 0.6893 | 2.074 | 1.151 | 2 | 1 |
| WCM156_Z5_1 | 13 | 115047220 | 115089411 | 0.1095 | 0.2802 | NA | NA | NA | NA |
| WCM156_Z5_1 | 14 | 19377710 | 33004964 | 0.2723 | 0.5555 | 1.9746 | 0.9648 | 2 | 1 |
| WCM156_Z5_1 | 14 | 33005011 | 107283027 | −0.0466 | −0.0069 | 1.9736 | 0.0169 | 2 | 0 |
| WCM156_Z5_1 | 15 | 20170091 | 35834654 | −0.0215 | 0.0411 | 1.3548 | 0.7029 | 1 | 1 |
| WCM156_Z5_1 | 15 | 36219092 | 54786879 | −0.4919 | −1.0416 | 0.962 | 0.0096 | 1 | 0 |
| WCM156_Z5_1 | 15 | 54792371 | 102517666 | −0.033 | 0.0192 | 1.0424 | 0.9843 | 1 | 1 |
| WCM156_Z5_1 | 16 | 67604 | 69972998 | −0.041 | 0.0038 | 1.018 | 0.9872 | 1 | 1 |
| WCM156_Z5_1 | 16 | 69973304 | 90233642 | −0.4661 | −0.9681 | 0.9945 | 0.0278 | 1 | 0 |
| WCM156_Z5_1 | 17 | 131595 | 14683390 | −0.479 | −1.0045 | 0.996 | 9.00E−04 | 1 | 0 |
| WCM156_Z5_1 | 17 | 15134287 | 71197439 | −0.0444 | −0.0026 | 1.0155 | 0.9809 | 1 | 1 |
| WCM156_Z5_1 | 17 | 71197707 | 81083507 | 0.3202 | 0.6328 | 2.018 | 1.083 | 2 | 1 |
| WCM156_Z5_1 | 18 | 48272 | 78005224 | −0.0435 | −0.001 | 1.0166 | 0.982 | 1 | 1 |
| WCM156_Z5_1 | 19 | 281467 | 59083951 | −0.0446 | −0.0031 | 1.0132 | 0.9825 | 1 | 1 |
| WCM156_Z5_1 | 2 | 41613 | 242946604 | −0.0454 | −0.0046 | 1.0103 | 0.9834 | 1 | 1 |
| WCM156_Z5_1 | 20 | 68373 | 3190210 | 0.3026 | 0.6046 | 2.0337 | 1.0074 | 2 | 1 |
| WCM156_Z5_1 | 20 | 3193978 | 62904845 | −0.0258 | 0.0328 | 1.0464 | 0.9997 | 1 | 1 |
| WCM156_Z5_1 | 21 | 9826235 | 39672220 | −0.055 | −0.0232 | 1.1938 | 0.7742 | 1 | 1 |
| WCM156_Z5_1 | 21 | 39755581 | 42870049 | −0.4392 | −0.8939 | 1.0715 | 0.0048 | 1 | 0 |
| WCM156_Z5_1 | 21 | 43133121 | 48084204 | −0.0181 | 0.0475 | 1.0687 | 0.9983 | 1 | 1 |
| WCM156_Z5_1 | 22 | 16287470 | 51219138 | −0.0485 | −0.0106 | 1.0157 | 0.9697 | 1 | 1 |
| WCM156_Z5_1 | 3 | 239443 | 117716044 | −0.0415 | 0.0029 | 1.0155 | 0.9886 | 1 | 1 |
| WCM156_Z5_1 | 3 | 118227497 | 171087418 | 0.276 | 0.5615 | 1.9268 | 1.0249 | 2 | 1 |
| WCM156_Z5_1 | 3 | 171321030 | 197894712 | −0.0591 | −0.031 | 1.0148 | 0.9426 | 1 | 1 |
| WCM156_Z5_1 | 4 | 59326 | 190426471 | −0.0551 | −0.0234 | 1.0049 | 0.9629 | 1 | 1 |
| WCM156_Z5_1 | 5 | 140532 | 66479914 | −0.0491 | −0.0118 | 1.0263 | 0.9574 | 1 | 1 |
| WCM156_Z5_1 | 5 | 66480145 | 94417174 | −0.5095 | −1.0932 | 0.9739 | −0.0365 | 1 | 0 |
| WCM156_Z5_1 | 5 | 94619615 | 180687804 | −0.047 | −0.0078 | 2.0129 | −0.0237 | 2 | 0 |
| WCM156_Z5_1 | 6 | 203542 | 367352 | 0.4145 | 0.7804 | NA | NA | NA | NA |
| WCM156_Z5_1 | 6 | 393317 | 32729453 | 0.5063 | 0.9192 | 2.9604 | 0.8217 | 3 | 1 |
| WCM156_Z5_1 | 6 | 32729693 | 108840919 | 0.2773 | 0.5637 | 1.9967 | 0.9594 | 2 | 1 |
| WCM156_Z5_1 | 6 | 108843583 | 119226912 | −0.5058 | −1.0825 | 0.9285 | 0.016 | 1 | 0 |
| WCM156_Z5_1 | 6 | 119228683 | 148664338 | 0.2682 | 0.5489 | 1.8161 | 1.1098 | 2 | 1 |
| WCM156_Z5_1 | 6 | 148701057 | 170893389 | −0.4759 | −0.9959 | 0.9858 | 0.0171 | 1 | 0 |
| WCM156_Z5_1 | 7 | 193265 | 72413593 | −0.0382 | 0.0093 | 1.0296 | 0.9833 | 1 | 1 |
| WCM156_Z5_1 | 7 | 72416688 | 84751079 | 0.282 | 0.5714 | 1.9774 | 0.9945 | 2 | 1 |
| WCM156_Z5_1 | 7 | 86274161 | 140912423 | −0.0555 | −0.0242 | 1.195 | 0.7717 | 1 | 1 |
| WCM156_Z5_1 | 7 | 141137460 | 158937375 | 0.2953 | 0.5928 | 1.9823 | 1.0341 | 2 | 1 |
| WCM156_Z5_1 | 8 | 190907 | 32621337 | −0.4372 | −0.8883 | 1.0861 | −0.0056 | 1 | 0 |
| WCM156_Z5_1 | 8 | 32621531 | 68939483 | −0.0464 | −0.0066 | 1.0587 | 0.9321 | 1 | 1 |
| WCM156_Z5_1 | 8 | 68942754 | 146279480 | 0.3028 | 0.6049 | 1.9803 | 1.0614 | 2 | 1 |
| WCM156_Z5_1 | 9 | 17073 | 141111414 | −0.0316 | 0.0219 | 1.0153 | 1.0153 | 1 | 1 |
| WCM156_Z5_1 | X | 2700122 | 2836047 | 0.079 | 0.2261 | NA | NA | NA | NA |
| WCM156_Z5_1 | X | 2836138 | 135961265 | −0.0891 | −0.0901 | NA | NA | NA | NA |
| WCM156_Z5_1 | X | 135961583 | 154509348 | 0.5129 | 0.929 | NA | NA | NA | NA |
| WCM158 | 1 | 14907 | 67207067 | 0.0758 | 0.1394 | 1.1014 | 1.1014 | 1 | 1 |
| WCM158 | 1 | 67208729 | 156314467 | −0.0032 | −0.2283 | 0.8537 | 0.8537 | 1 | 1 |
| WCM158 | 1 | 156314539 | 200635440 | 0.0618 | 0.0792 | 1.0564 | 1.0564 | 1 | 1 |
| WCM158 | 1 | 200635617 | 207651294 | 0.2014 | 0.6078 | 1.5239 | 1.5239 | 2 | 2 |
| WCM158 | 1 | 207651437 | 249213967 | 0.0076 | −0.1733 | 0.8868 | 0.8868 | 1 | 1 |
| WCM158 | 10 | 226100 | 135438946 | 0.0517 | 0.0345 | 1.0242 | 1.0242 | 1 | 1 |
| WCM158 | 11 | 193112 | 18045429 | 0.0941 | 0.2148 | 1.1605 | 1.1605 | 1 | 1 |
| WCM158 | 11 | 18047284 | 117152292 | −0.012 | −0.2743 | 0.8268 | 0.8268 | 1 | 1 |
| WCM158 | 11 | 117152674 | 134605954 | 0.0926 | 0.2089 | 1.1558 | 1.1558 | 1 | 1 |
| WCM158 | 12 | 148444 | 51851126 | 0.0409 | −0.0139 | 0.9904 | 0.9904 | 1 | 1 |
| WCM158 | 12 | 51852444 | 53346667 | 0.2767 | 0.8443 | 1.7954 | 1.7954 | 2 | 2 |
| WCM158 | 12 | 53400263 | 133810780 | −0.0152 | −0.2911 | 0.8173 | 0.8173 | 1 | 1 |
| WCM158 | 13 | 19042044 | 115089411 | 0.0092 | −0.1656 | 0.8915 | 0.8915 | 1 | 1 |
| WCM158 | 14 | 19110188 | 107283266 | 0.0193 | −0.116 | 0.9228 | 0.9228 | 1 | 1 |
| WCM158 | 15 | 20083910 | 102514187 | 0.065 | 0.0933 | 1.0668 | 1.0668 | 1 | 1 |
| WCM158 | 16 | 64396 | 90260461 | −0.019 | −0.3118 | 0.8056 | 0.8056 | 1 | 1 |
| WCM158 | 17 | 131595 | 81187855 | 0.0297 | −0.0661 | 0.9552 | 0.9552 | 1 | 1 |
| WCM158 | 18 | 47725 | 78005282 | 0.0223 | −0.1014 | 0.9321 | 0.9321 | 1 | 1 |
| WCM158 | 19 | 105050 | 59093645 | −0.07 | −0.6112 | 0.6547 | 0.6547 | 1 | 1 |
| WCM158 | 2 | 41609 | 243052790 | 0.0187 | −0.1187 | 0.921 | 0.921 | 1 | 1 |
| WCM158 | 20 | 68375 | 62934572 | 0.0478 | 0.0172 | 1.012 | 1.012 | 1 | 1 |
| WCM158 | 21 | 9590370 | 42080352 | −0.0392 | −0.4243 | 0.7452 | 0.7452 | 1 | 1 |
| WCM158 | 21 | 42080666 | 47639391 | 0.1809 | 0.5385 | 1.4525 | 1.4525 | 1 | 1 |
| WCM158 | 21 | 47641788 | 48084160 | −0.1233 | −0.993 | NA | NA | NA | NA |
| WCM158 | 22 | 16159016 | 51219090 | 0.0835 | 0.1715 | 1.1262 | 1.1262 | 1 | 1 |
| WCM158 | 3 | 239451 | 197896800 | 0.0217 | −0.104 | 0.9304 | 0.9304 | 1 | 1 |
| WCM158 | 4 | 53363 | 190878508 | −0.0011 | −0.2175 | 0.86 | 0.86 | 1 | 1 |
| WCM158 | 5 | 90697 | 139884482 | −0.0092 | −0.2593 | 0.8355 | 0.8355 | 1 | 1 |
| WCM158 | 5 | 139884860 | 140799205 | −0.2664 | −3.0565 | 0.2228 | 0.0176 | 0 | 0 |
| WCM158 | 5 | 140799403 | 180687812 | 0.0431 | −0.0041 | 0.9971 | 0.9971 | 1 | 1 |
| WCM158 | 6 | 203424 | 170892854 | 0.0184 | −0.12 | 0.9202 | 0.9202 | 1 | 1 |
| WCM158 | 7 | 193189 | 158937074 | 0.0039 | −0.1919 | 0.8755 | 0.8755 | 1 | 1 |
| WCM158 | 8 | 163457 | 146279441 | 0.006 | −0.1816 | 0.8817 | 0.8817 | 1 | 1 |
| WCM158 | 9 | 14859 | 141107535 | 0.043 | −0.0046 | 0.9968 | 0.9968 | 1 | 1 |
| WCM158 | X | 2700139 | 154493576 | −0.1172 | −0.9452 | NA | NA | NA | NA |
| WCM158_X1_1 | 1 | 721439 | 1289006 | 0.0934 | 1.0785 | 3.0498 | 1.1739 | 3 | 1 |
| WCM158_X1_1 | 1 | 1289427 | 2526275 | −0.0565 | 0.7428 | 1.8097 | 1.5372 | 2 | 2 |
| WCM158_X1_1 | 1 | 2526746 | 12157172 | −0.2809 | 0.1326 | 2.0291 | 0.1634 | 2 | 0 |
| WCM158_X1_1 | 1 | 12164521 | 120612005 | −0.1233 | 0.5773 | 1.9741 | 1.0101 | 2 | 1 |
| WCM158_X1_1 | 1 | 143767848 | 249212420 | 0.2079 | 1.3098 | 2.9795 | 1.9786 | 3 | 2 |
| WCM158_X1_1 | 10 | 225995 | 3151636 | −0.3272 | −0.0176 | NA | NA | NA | NA |
| WCM158_X1_1 | 10 | 3154460 | 42916228 | 4.00E−04 | 0.8755 | 1.943 | 1.7262 | 2 | 2 |
| WCM158_X1_1 | 10 | 42920832 | 59956205 | −0.0567 | 0.7421 | 3.5554 | −0.2102 | 4 | 0 |
| WCM158_X1_1 | 10 | 59956439 | 88836391 | 0.2111 | 1.3162 | 3.6911 | 1.289 | 4 | 1 |
| WCM158_X1_1 | 10 | 88853704 | 89550161 | −0.089 | 0.6637 | NA | NA | NA | NA |
| WCM158_X1_1 | 10 | 89552500 | 105039713 | −0.325 | −0.0102 | 2.1172 | −0.1313 | 2 | 0 |
| WCM158_X1_1 | 10 | 105046794 | 105128296 | 0.1119 | 1.1172 | NA | NA | NA | NA |
| WCM158_X1_1 | 10 | 105133344 | 123727226 | 0.284 | 1.4541 | 2.9884 | 2.4912 | 3 | 2 |
| WCM158_X1_1 | 10 | 123733594 | 135440090 | 0.1566 | 1.2086 | 3.5651 | 1.0571 | 4 | 1 |
| WCM158_X1_1 | 11 | 193151 | 1017893 | 0.1311 | 1.1569 | 2.8914 | 1.5681 | 3 | 2 |
| WCM158_X1_1 | 11 | 1018295 | 1575841 | 0.2238 | 1.3406 | 3.3987 | 1.6665 | 3 | 2 |
| WCM158_X1_1 | 11 | 1576205 | 134856562 | 0.0486 | 0.9827 | 2.0322 | 1.9201 | 2 | 2 |
| WCM158_X1_1 | 12 | 148637 | 10224112 | 0.1049 | 1.1026 | 2.264 | 2.031 | 2 | 2 |
| WCM158_X1_1 | 12 | 10225907 | 18234256 | −0.3448 | −0.0778 | 2.145 | −0.25 | 2 | 0 |
| WCM158_X1_1 | 12 | 18234384 | 21448664 | −0.9182 | NA | 0.059 | −0.3159 | 0 | 0 |
| WCM158_X1_1 | 12 | 21450349 | 23687226 | −0.3261 | −0.0139 | NA | NA | NA | NA |
| WCM158_X1_1 | 12 | 23687449 | 33576943 | 0.0131 | 0.904 | 1.9721 | 1.7703 | 2 | 2 |
| WCM158_X1_1 | 12 | 33579278 | 41903632 | −0.4591 | −0.5194 | 1.4069 | −0.0116 | 1 | 0 |
| WCM158_X1_1 | 12 | 41946539 | 96377687 | 0.0318 | 0.9457 | 2.0028 | 1.8494 | 2 | 2 |
| WCM158_X1_1 | 12 | 96379914 | 131296908 | 0.0718 | 1.0327 | 3.2239 | 0.8679 | 3 | 1 |
| WCM158_X1_1 | 12 | 131306314 | 133810874 | −0.1188 | 0.5888 | 2.1563 | 0.8516 | 2 | 1 |
| WCM158_X1_1 | 13 | 19600159 | 39550828 | −0.1422 | 0.5282 | 1.7404 | 1.1439 | 2 | 1 |
| WCM158_X1_1 | 13 | 39550924 | 102051466 | −0.2997 | 0.0728 | 1.959 | 0.1446 | 2 | 0 |
| WCM158_X1_1 | 13 | 102105212 | 115089411 | −0.169 | 0.457 | 1.5951 | 1.1503 | 2 | 1 |
| WCM158_X1_1 | 14 | 19377719 | 20389264 | −0.1192 | 0.5879 | NA | NA | NA | NA |
| WCM158_X1_1 | 14 | 20389476 | 39650473 | −0.0883 | 0.6654 | 3.0715 | 0.1006 | 3 | 0 |
| WCM158_X1_1 | 14 | 39650733 | 107283063 | 0.027 | 0.9352 | 3.0075 | 0.8168 | 3 | 1 |
| WCM158_X1_1 | 15 | 20170150 | 102517667 | −0.1245 | 0.5742 | 1.9743 | 1.0034 | 2 | 1 |
| WCM158_X1_1 | 16 | 66953 | 135678 | −0.2719 | 0.1605 | NA | NA | NA | NA |
| WCM158_X1_1 | 16 | 136772 | 46655205 | −0.2742 | 0.1532 | 1.1499 | 1.0741 | 1 | 1 |
| WCM158_X1_1 | 16 | 46660579 | 69660315 | −0.0829 | 0.6786 | 1.7501 | 1.4511 | 2 | 1 |
| WCM158_X1_1 | 16 | 69678710 | 70508797 | −0.2662 | 0.178 | NA | NA | NA | NA |
| WCM158_X1_1 | 16 | 70509327 | 90233627 | −0.2744 | 0.1528 | 2.1108 | 0.1127 | 2 | 0 |
| WCM158_X1_1 | 17 | 131595 | 21191346 | −0.2996 | 0.0731 | 2.0709 | 0.033 | 2 | 0 |
| WCM158_X1_1 | 17 | 21194843 | 81083455 | 0.0783 | 1.0466 | 2.1184 | 2.0128 | 2 | 2 |
| WCM158_X1_1 | 18 | 49000 | 78005210 | 0.0366 | 0.9563 | 3.0173 | 0.8634 | 3 | 1 |
| WCM158_X1_1 | 19 | 281503 | 5623236 | −0.1048 | 0.6242 | 2.0954 | 0.9873 | 2 | 1 |
| WCM158_X1_1 | 19 | 5626460 | 19768190 | 0.0664 | 1.0213 | 2.0559 | 2.0035 | 2 | 2 |
| WCM158_X1_1 | 19 | 19770501 | 33414392 | −0.058 | 0.739 | 2.0893 | 1.2488 | 2 | 1 |
| WCM158_X1_1 | 19 | 33417117 | 59083929 | 0.0545 | 0.9955 | 2.9587 | 1.0288 | 3 | 1 |
| WCM158_X1_1 | 2 | 41609 | 32419023 | 0.0386 | 0.9608 | 2.0362 | 1.8565 | 2 | 2 |
| WCM158_X1_1 | 2 | 32422422 | 58311262 | −0.3288 | −0.023 | 1.8998 | 0.0685 | 2 | 0 |
| WCM158_X1_1 | 2 | 58313555 | 89442316 | 0.0494 | 0.9844 | 2.0043 | 1.9526 | 2 | 2 |
| WCM158_X1_1 | 2 | 89475873 | 115822349 | −0.1094 | 0.6128 | 2.2012 | 0.8571 | 2 | 1 |
| WCM158_X1_1 | 2 | 115919825 | 141474380 | −0.5362 | −0.8893 | 1.1145 | −0.0348 | 1 | 0 |
| WCM158_X1_1 | 2 | 141526813 | 242946616 | −0.1373 | 0.5412 | 1.9249 | 0.9854 | 2 | 1 |
| WCM158_X1_1 | 20 | 68351 | 62904932 | −0.0664 | 0.7188 | 2.2546 | 1.037 | 2 | 1 |
| WCM158_X1_1 | 21 | 9826207 | 48084282 | 0.0665 | 1.0214 | 2.0823 | 1.9776 | 2 | 2 |
| WCM158_X1_1 | 22 | 16287495 | 32554985 | −0.3018 | 0.0661 | 2.0271 | 0.0666 | 2 | 0 |
| WCM158_X1_1 | 22 | 32579559 | 51064080 | −0.1158 | 0.5964 | 2.008 | 1.0159 | 2 | 1 |
| WCM158_X1_1 | 22 | 51064485 | 51215157 | −0.2745 | 0.1524 | NA | NA | NA | NA |
| WCM158_X1_1 | 3 | 239349 | 431068 | 0.1935 | 1.2817 | NA | NA | NA | NA |
| WCM158_X1_1 | 3 | 432418 | 13679203 | 0.2929 | 1.4706 | 3.629 | 1.9138 | 4 | 2 |
| WCM158_X1_1 | 3 | 13679335 | 47614274 | 0.278 | 1.443 | 3.6838 | 1.7539 | 4 | 2 |
| WCM158_X1_1 | 3 | 47618447 | 56026216 | 0.2016 | 1.2976 | 2.6307 | 2.2857 | 3 | 2 |
| WCM158_X1_1 | 3 | 56041300 | 89528635 | 0.0623 | 1.0124 | 2.6569 | 1.3778 | 3 | 1 |
| WCM158_X1_1 | 3 | 93595943 | 97454773 | 0.3973 | 1.6573 | NA | NA | NA | NA |
| WCM158_X1_1 | 3 | 97464222 | 148881712 | 0.4098 | 1.679 | 5.4715 | 0.9325 | 5 | 1 |
| WCM158_X1_1 | 3 | 148883833 | 197896711 | 0.2892 | 1.4637 | 5.36 | 0.1564 | 5 | 0 |
| WCM158_X1_1 | 4 | 59390 | 118006371 | −0.1045 | 0.625 | 2.0654 | 1.019 | 2 | 1 |
| WCM158_X1_1 | 4 | 118006445 | 138442774 | −0.3287 | −0.0226 | 2.003 | −0.0341 | 2 | 0 |
| WCM158_X1_1 | 4 | 138449683 | 190426502 | −0.1617 | 0.4766 | 1.9315 | 0.8513 | 2 | 1 |
| WCM158_X1_1 | 5 | 140636 | 64748682 | −0.1605 | 0.4799 | 1.8233 | 0.9659 | 2 | 1 |
| WCM158_X1_1 | 5 | 64756164 | 82360910 | −1.00E−04 | 0.8741 | 2.6828 | 0.983 | 3 | 1 |
| WCM158_X1_1 | 5 | 82373142 | 96519257 | −0.181 | 0.4244 | 2.7423 | −0.0583 | 3 | 0 |
| WCM158_X1_1 | 5 | 98109791 | 107197473 | −0.8747 | NA | NA | NA | NA | NA |
| WCM158_X1_1 | 5 | 107216644 | 112102993 | −0.1836 | 0.4173 | 2.5938 | 0.077 | 3 | 0 |
| WCM158_X1_1 | 5 | 112116552 | 180687769 | 0.0261 | 0.9332 | 2.9405 | 0.8785 | 3 | 1 |
| WCM158_X1_1 | 6 | 203699 | 64390043 | −0.0064 | 0.8598 | 2.7108 | 0.9188 | 3 | 1 |
| WCM158_X1_1 | 6 | 64394035 | 70745815 | −0.2084 | 0.348 | NA | NA | NA | NA |
| WCM158_X1_1 | 6 | 70769669 | 96997436 | −0.4305 | −0.3991 | 1.1479 | 0.3688 | 1 | 0 |
| WCM158_X1_1 | 6 | 96997629 | 170893450 | −0.1597 | 0.4819 | 1.8666 | 0.9266 | 2 | 1 |
| WCM158_X1_1 | 7 | 193222 | 158937046 | 0.2707 | 1.4294 | 2.7487 | 2.6381 | 3 | 3 |
| WCM158_X1_1 | 8 | 190900 | 607779 | −0.5608 | −1.0252 | NA | NA | NA | NA |
| WCM158_X1_1 | 8 | 608034 | 12888899 | −0.5406 | −0.9128 | 0.9093 | 0.153 | 1 | 0 |
| WCM158_X1_1 | 8 | 12943356 | 35425672 | −0.3178 | 0.0136 | 1.9087 | 0.1102 | 2 | 0 |
| WCM158_X1_1 | 8 | 35453112 | 51664666 | 0.2309 | 1.3542 | 5.0845 | 0.0285 | 5 | 0 |
| WCM158_X1_1 | 8 | 51705264 | 71520327 | −0.1354 | 0.546 | 2.8029 | 0.1172 | 3 | 0 |
| WCM158_X1_1 | 8 | 71550105 | 146279489 | 0.211 | 1.3159 | 5.0457 | −0.0666 | 5 | 0 |
| WCM158_X1_1 | 9 | 17074 | 23705016 | −0.2068 | 0.3525 | 1.4104 | 1.1432 | 1 | 1 |
| WCM158_X1_1 | 9 | 23731032 | 140852095 | 0.0566 | 1.0001 | 2.9361 | 1.0641 | 3 | 1 |
| WCM158_X1_1 | 9 | 140865989 | 141111414 | −0.0524 | 0.7526 | NA | NA | NA | NA |
| WCM158_X1_1 | X | 2700151 | 46539182 | −0.0855 | 0.6722 | NA | NA | NA | NA |
| WCM158_X1_1 | X | 46541841 | 52681330 | −0.3961 | −0.2638 | NA | NA | NA | NA |
| WCM158_X1_1 | X | 52825534 | 136113442 | 0.0154 | 0.9093 | NA | NA | NA | NA |
| WCM158_X1_1 | X | 136113784 | 140969584 | −0.788 | −3.6492 | NA | NA | NA | NA |
| WCM158_X1_1 | X | 140982567 | 154509348 | 0.0639 | 1.0157 | NA | NA | NA | NA |
| WCM15_Z1_1 | 1 | 808999 | 10408784 | 0.0876 | 0.0144 | 1.0197 | 1.0004 | 1 | 1 |
| WCM15_Z1_1 | 1 | 10412700 | 12780904 | −0.7759 | −0.9257 | 1.06 | −0.0072 | 1 | 0 |
| WCM15_Z1_1 | 1 | 12785303 | 249212579 | 0.087 | 0.0139 | 1.0097 | 1.0097 | 1 | 1 |
| WCM15_Z1_1 | 10 | 225952 | 44876365 | 0.0595 | −0.0154 | 0.9989 | 0.9799 | 1 | 1 |
| WCM15_Z1_1 | 10 | 45406998 | 62149194 | −0.8055 | −0.959 | 1.0314 | −0.0025 | 1 | 0 |
| WCM15_Z1_1 | 10 | 62539896 | 74774057 | 0.0981 | 0.0256 | 1.0244 | 1.0114 | 1 | 1 |
| WCM15_Z1_1 | 10 | 74776587 | 76603080 | −0.815 | −0.9696 | 1.0173 | 0.004 | 1 | 0 |
| WCM15_Z1_1 | 10 | 76603125 | 88213604 | 0.0656 | −0.0089 | 1.0203 | 0.9674 | 1 | 1 |
| WCM15_Z1_1 | 10 | 88218761 | 88696746 | −0.7971 | −0.9496 | NA | NA | NA | NA |
| WCM15_Z1_1 | 10 | 88702220 | 90774134 | −3.5124 | −5.3373 | NA | NA | NA | NA |
| WCM15_Z1_1 | 10 | 90966393 | 95279565 | −0.7851 | −0.9361 | 1.0634 | −0.0181 | 1 | 0 |
| WCM15_Z1_1 | 10 | 95287873 | 105093694 | 0.087 | 0.0138 | 1.0204 | 0.9989 | 1 | 1 |
| WCM15_Z1_1 | 10 | 105104833 | 106163525 | −0.8342 | −0.9913 | 1.0047 | 0.0014 | 1 | 0 |
| WCM15_Z1_1 | 10 | 106166524 | 135373678 | 0.0371 | −0.0393 | 0.9731 | 0.9731 | 1 | 1 |
| WCM15_Z1_1 | 11 | 193096 | 134257436 | 0.0624 | −0.0123 | 0.9957 | 0.9873 | 1 | 1 |
| WCM15_Z1_1 | 12 | 247582 | 11548375 | −0.8181 | −0.9731 | 1.0299 | −0.0111 | 1 | 0 |
| WCM15_Z1_1 | 12 | 11803048 | 12871051 | −3.5811 | −5.6001 | NA | NA | NA | NA |
| WCM15_Z1_1 | 12 | 12917552 | 14019034 | −0.8244 | −0.9802 | NA | NA | NA | NA |
| WCM15_Z1_1 | 12 | 14577059 | 15073853 | 0.801 | 0.763 | NA | NA | NA | NA |
| WCM15_Z1_1 | 12 | 15087898 | 133779249 | 0.0834 | 0.01 | 1.0091 | 1.0048 | 1 | 1 |
| WCM15_Z1_1 | 13 | 19747959 | 21751210 | −0.3668 | −0.4741 | NA | NA | NA | NA |
| WCM15_Z1_1 | 13 | 21751271 | 48835337 | 0.6114 | 0.5658 | 2.9625 | −0.0021 | 3 | 0 |
| WCM15_Z1_1 | 13 | 48878001 | 115089411 | −0.8513 | −1.0106 | 0.9932 | −5.00E−04 | 1 | 0 |
| WCM15_Z1_1 | 14 | 20215851 | 107283088 | 0.634 | 0.5894 | 1.9955 | 1.0137 | 2 | 1 |
| WCM15_Z1_1 | 15 | 20170133 | 24921462 | −0.0323 | −0.1133 | 1.465 | 0.3839 | 1 | 0 |
| WCM15_Z1_1 | 15 | 24921771 | 28525361 | −0.0531 | −0.1356 | 0.9308 | 0.8899 | 1 | 1 |
| WCM15_Z1_1 | 15 | 28900819 | 59813441 | −0.3119 | −0.4145 | 0.9725 | 0.528 | 1 | 1 |
| WCM15_Z1_1 | 15 | 59910425 | 102359167 | 0.0802 | 0.0066 | 1.496 | 0.5131 | 1 | 1 |
| WCM15_Z1_1 | 16 | 97610 | 67335734 | −0.8077 | −0.9615 | 1.0274 | −3.00E−04 | 1 | 0 |
| WCM15_Z1_1 | 16 | 67337200 | 67943642 | 0.4085 | 0.3534 | NA | NA | NA | NA |
| WCM15_Z1_1 | 16 | 67961447 | 90161760 | −0.8129 | −0.9673 | 1.0287 | −0.0057 | 1 | 0 |
| WCM15_Z1_1 | 17 | 131595 | 16097787 | 0.0345 | −0.042 | 1.9443 | −0.0017 | 2 | 0 |
| WCM15_Z1_1 | 17 | 16120623 | 21188298 | −0.8376 | −0.9951 | 1.0044 | −0.0011 | 1 | 0 |
| WCM15_Z1_1 | 17 | 21201719 | 81052325 | 0.0695 | −0.0048 | 1.002 | 0.9914 | 1 | 1 |
| WCM15_Z1_1 | 18 | 47646 | 78005197 | −0.3082 | −0.4104 | 1.5115 | −0.0067 | 2 | 0 |
| WCM15_Z1_1 | 19 | 281565 | 11436181 | 0.0194 | −0.0582 | 0.9646 | 0.9564 | 1 | 1 |
| WCM15_Z1_1 | 19 | 11446316 | 14294396 | −0.8891 | −1.0533 | 0.9588 | 0.0049 | 1 | 0 |
| WCM15_Z1_1 | 19 | 14492263 | 15807807 | 0.0209 | −0.0566 | 0.9728 | 0.9503 | 1 | 1 |
| WCM15_Z1_1 | 19 | 15838925 | 59082752 | 0.5914 | 0.5449 | 1.9371 | 0.9808 | 2 | 1 |
| WCM15_Z1_1 | 2 | 41583 | 61420113 | −0.3003 | −0.4019 | 0.9949 | 0.5189 | 1 | 1 |
| WCM15_Z1_1 | 2 | 61430344 | 62132964 | −0.8124 | −0.9667 | NA | NA | NA | NA |
| WCM15_Z1_1 | 2 | 62227997 | 110373618 | −0.3034 | −0.4052 | 0.9893 | 0.5209 | 1 | 1 |
| WCM15_Z1_1 | 2 | 110881513 | 112786476 | −1.5668 | −1.8504 | NA | NA | NA | NA |
| WCM15_Z1_1 | 2 | 112817600 | 174232341 | −0.2883 | −0.3889 | 1.0062 | 0.5213 | 1 | 1 |
| WCM15_Z1_1 | 2 | 174774863 | 176046152 | −1.5922 | −1.8817 | NA | NA | NA | NA |
| WCM15_Z1_1 | 2 | 176794779 | 242815461 | −0.2973 | −0.3985 | 1.0004 | 0.5168 | 1 | 1 |
| WCM15_Z1_1 | 20 | 68336 | 26188809 | −0.6045 | −0.7349 | 1.2037 | −0.002 | 1 | 0 |
| WCM15_Z1_1 | 20 | 29845559 | 62904835 | 0.2149 | 0.1495 | 1.1314 | 1.0869 | 1 | 1 |
| WCM15_Z1_1 | 21 | 10862667 | 48084270 | 0.0508 | −0.0247 | 0.9883 | 0.9778 | 1 | 1 |
| WCM15_Z1_1 | 22 | 17072636 | 38485064 | −0.8198 | −0.9751 | 1.0183 | −9.00E−04 | 1 | 0 |
| WCM15_Z1_1 | 22 | 38485716 | 38710157 | −3.4227 | −5.0392 | NA | NA | NA | NA |
| WCM15_Z1_1 | 22 | 38822894 | 43582988 | −0.8218 | −0.9773 | 1.0271 | −0.0112 | 1 | 0 |
| WCM15_Z1_1 | 22 | 43600006 | 44131712 | −3.6181 | −5.7582 | NA | NA | NA | NA |
| WCM15_Z1_1 | 22 | 44151646 | 51216336 | −0.8171 | −0.972 | 1.0243 | −0.0047 | 1 | 0 |
| WCM15_Z1_1 | 3 | 367645 | 197765432 | 0.0902 | 0.0173 | 1.012 | 1.012 | 1 | 1 |
| WCM15_Z1_1 | 4 | 53340 | 27162532 | −0.8554 | −1.0151 | 0.9919 | −0.0024 | 1 | 0 |
| WCM15_Z1_1 | 4 | 30723155 | 77134535 | −0.3115 | −0.414 | 0.99 | 0.5111 | 1 | 1 |
| WCM15_Z1_1 | 4 | 77228029 | 85504535 | −1.615 | −1.9099 | 0.5472 | −0.015 | 1 | 0 |
| WCM15_Z1_1 | 4 | 85525527 | 189068520 | −0.2997 | −0.4012 | 0.9929 | 0.5216 | 1 | 1 |
| WCM15_Z1_1 | 5 | 140663 | 50689286 | 0.4385 | 0.3849 | 1.4661 | 1.1456 | 1 | 1 |
| WCM15_Z1_1 | 5 | 52096290 | 148207663 | −0.2829 | −0.383 | 1.5394 | −0.0057 | 2 | 0 |
| WCM15_Z1_1 | 5 | 148384455 | 149265875 | 0.8371 | 0.8005 | 1.9601 | 1.5232 | 2 | 2 |
| WCM15_Z1_1 | 5 | 149274826 | 149932950 | 0.4177 | 0.3631 | 1.5305 | 1.0418 | 2 | 1 |
| WCM15_Z1_1 | 5 | 150027818 | 153857582 | 0.8753 | 0.84 | 2.0347 | 1.5453 | 2 | 2 |
| WCM15_Z1_1 | 5 | 154092540 | 180687824 | 0.4395 | 0.386 | 1.4781 | 1.1354 | 1 | 1 |
| WCM15_Z1_1 | 6 | 292575 | 18465260 | 0.0785 | 0.0048 | 1.0194 | 0.9874 | 1 | 1 |
| WCM15_Z1_1 | 6 | 18572160 | 28472253 | −0.8603 | −1.0207 | 0.9922 | −0.0065 | 1 | 0 |
| WCM15_Z1_1 | 6 | 28473566 | 170892853 | 0.0791 | 0.0054 | 1.008 | 0.9995 | 1 | 1 |
| WCM15_Z1_1 | 7 | 193687 | 16918167 | −0.7718 | −0.9211 | 1.0626 | −0.0064 | 1 | 0 |
| WCM15_Z1_1 | 7 | 17338900 | 18633661 | 1.7016 | 1.6886 | NA | NA | NA | NA |
| WCM15_Z1_1 | 7 | 18669158 | 45614757 | 0.097 | 0.0245 | 1.0312 | 1.0031 | 1 | 1 |
| WCM15_Z1_1 | 7 | 45632498 | 131982831 | −0.0136 | −0.0934 | 1.0014 | 0.8733 | 1 | 1 |
| WCM15_Z1_1 | 7 | 132070054 | 158937481 | 0.5529 | 0.5047 | 1.9674 | 0.8702 | 2 | 1 |
| WCM15_Z1_1 | 8 | 190890 | 681206 | −0.0167 | −0.0967 | NA | NA | NA | NA |
| WCM15_Z1_1 | 8 | 1496890 | 31498232 | −0.8319 | −0.9887 | 1.0149 | −0.0071 | 1 | 0 |
| WCM15_Z1_1 | 8 | 32406274 | 146279472 | 0.057 | −0.0181 | 0.9875 | 0.9875 | 1 | 1 |
| WCM15_Z1_1 | 9 | 214625 | 66517198 | −0.2877 | −0.3882 | 1.0365 | 0.4917 | 1 | 0 |
| WCM15_Z1_1 | 9 | 71144570 | 78547330 | −1.6631 | −1.9698 | 0.502 | 0.0086 | 1 | 0 |
| WCM15_Z1_1 | 9 | 78601149 | 141016415 | −0.2946 | −0.3956 | 1.0189 | 0.5014 | 1 | 1 |
| WCM15_Z1_1 | X | 2700119 | 25033756 | 1.3114 | 1.2895 | NA | NA | NA | NA |
| WCM15_Z1_1 | X | 26157133 | 154507335 | 0.5804 | 0.5334 | NA | NA | NA | NA |
| WCM160 | 1 | 14907 | 62444854 | −0.0242 | −0.1584 | 0.896 | 0.896 | 1 | 1 |
| WCM160 | 1 | 62445467 | 117618067 | −0.1379 | −1.2173 | 0.4301 | 0.4301 | 0 | 0 |
| WCM160 | 1 | 117618402 | 145475385 | −0.0494 | −0.3407 | 1.0131 | 0.5662 | 1 | 1 |
| WCM160 | 1 | 145487351 | 156670737 | 0.2857 | 1.2443 | 2.369 | 2.369 | 2 | 2 |
| WCM160 | 1 | 156670822 | 228456529 | 0.096 | 0.5162 | 1.4302 | 1.4302 | 1 | 1 |
| WCM160 | 1 | 228459663 | 228601638 | 0.3421 | 1.4181 | 2.6724 | 2.6724 | 3 | 3 |
| WCM160 | 1 | 228602501 | 249214028 | −0.0651 | −0.4651 | 0.7244 | 0.7244 | 1 | 1 |
| WCM160 | 10 | 226060 | 135477933 | −0.0362 | −0.2429 | 0.8451 | 0.8451 | 1 | 1 |
| WCM160 | 11 | 193112 | 61717854 | 0.0748 | 0.4143 | 1.3326 | 1.3326 | 1 | 1 |
| WCM160 | 11 | 61719237 | 65836130 | −0.0813 | −0.6051 | 0.6574 | 0.6574 | 1 | 1 |
| WCM160 | 11 | 65961017 | 107526694 | 0.1363 | 0.6954 | 1.6194 | 1.6194 | 2 | 2 |
| WCM160 | 11 | 107535902 | 126310358 | −0.0993 | −0.7749 | 0.6544 | 0.5144 | 1 | 1 |
| WCM160 | 11 | 126314948 | 134605906 | 0.1214 | 0.6309 | 1.5486 | 1.5486 | 2 | 2 |
| WCM160 | 12 | 148468 | 133810703 | −0.0231 | −0.1508 | 0.9008 | 0.9008 | 1 | 1 |
| WCM160 | 13 | 19041937 | 115089411 | −0.0809 | −0.6017 | 0.659 | 0.659 | 1 | 1 |
| WCM160 | 14 | 19110188 | 107283269 | −0.0135 | −0.0868 | 0.9416 | 0.9416 | 1 | 1 |
| WCM160 | 15 | 20083889 | 35235505 | 0.1047 | 0.5563 | 1.5444 | 1.3965 | 2 | 1 |
| WCM160 | 15 | 35236651 | 102291971 | −0.0347 | −0.2316 | 0.8517 | 0.8517 | 1 | 1 |
| WCM160 | 15 | 102292673 | 102514248 | −0.0399 | −0.2695 | NA | NA | NA | NA |
| WCM160 | 16 | 66868 | 304565 | 0.0841 | 0.4594 | NA | NA | NA | NA |
| WCM160 | 16 | 304661 | 19234465 | 0.1958 | 0.9324 | 1.937 | 1.8798 | 2 | 2 |
| WCM160 | 16 | 19236063 | 28950223 | 0.423 | 1.6459 | 3.7243 | 2.5345 | 4 | 3 |
| WCM160 | 16 | 28962488 | 30046763 | 0.2265 | 1.0444 | 4.2343 | −0.1093 | 4 | 0 |
| WCM160 | 16 | 30064826 | 90260461 | −0.0846 | −0.6349 | 0.7088 | 0.5792 | 1 | 1 |
| WCM160 | 17 | 131595 | 10097546 | −0.1787 | −1.879 | 0.4707 | 0.0731 | 0 | 0 |
| WCM160 | 17 | 10204269 | 29704153 | 0.0993 | 0.5315 | 1.4455 | 1.4455 | 1 | 1 |
| WCM160 | 17 | 29704421 | 37902376 | 0.423 | 1.6459 | 3.9339 | 2.325 | 4 | 2 |
| WCM160 | 17 | 37903063 | 81187883 | −0.0245 | −0.1607 | 0.8946 | 0.8946 | 1 | 1 |
| WCM160 | 18 | 47666 | 78005150 | −0.0108 | −0.0686 | 0.9535 | 0.9535 | 1 | 1 |
| WCM160 | 19 | 104935 | 59093645 | −0.0483 | −0.332 | 0.7944 | 0.7944 | 1 | 1 |
| WCM160 | 2 | 41595 | 275178 | 0.0235 | 0.1411 | NA | NA | NA | NA |
| WCM160 | 2 | 276942 | 202150042 | −0.0507 | −0.3509 | 0.8082 | 0.76 | 1 | 1 |
| WCM160 | 2 | 202151148 | 243037103 | 0.0593 | 0.3359 | 1.2622 | 1.2622 | 1 | 1 |
| WCM160 | 20 | 68319 | 62934618 | 0.104 | 0.5531 | 1.4672 | 1.4672 | 1 | 1 |
| WCM160 | 21 | 9590356 | 48084189 | −0.1532 | −1.4333 | 0.3703 | 0.3703 | 0 | 0 |
| WCM160 | 22 | 16159060 | 51223730 | 3.00E−04 | 0.0021 | 1.0014 | 1.0014 | 1 | 1 |
| WCM160 | 3 | 239390 | 197896638 | −0.0271 | −0.1785 | 0.8836 | 0.8836 | 1 | 1 |
| WCM160 | 4 | 53345 | 49601579 | −0.0328 | −0.2182 | 0.8596 | 0.8596 | 1 | 1 |
| WCM160 | 4 | 52729276 | 100851835 | 0.0941 | 0.5074 | 1.4348 | 1.4081 | 1 | 1 |
| WCM160 | 4 | 100867759 | 101045932 | −1.4365 | NA | NA | NA | NA | NA |
| WCM160 | 4 | 101046788 | 184241550 | 0.0556 | 0.3167 | 1.278 | 1.213 | 1 | 1 |
| WCM160 | 4 | 184241811 | 190878479 | −0.008 | −0.0505 | 1.0091 | 0.9221 | 1 | 1 |
| WCM160 | 5 | 90647 | 37057454 | 0.0524 | 0.3 | 1.2311 | 1.2311 | 1 | 1 |
| WCM160 | 5 | 37058956 | 145562047 | −0.066 | −0.4732 | 0.7204 | 0.7204 | 1 | 1 |
| WCM160 | 5 | 145583278 | 180687854 | 0.0435 | 0.2526 | 1.1913 | 1.1913 | 1 | 1 |
| WCM160 | 6 | 203542 | 33545262 | 0.0329 | 0.1944 | 1.1442 | 1.1442 | 1 | 1 |
| WCM160 | 6 | 33553455 | 65016935 | 0.1392 | 0.7077 | 1.6332 | 1.6332 | 2 | 2 |
| WCM160 | 6 | 65097346 | 170892883 | −0.0391 | −0.264 | 0.8328 | 0.8328 | 1 | 1 |
| WCM160 | 7 | 193205 | 158937158 | 0.0343 | 0.202 | 1.1503 | 1.1503 | 1 | 1 |
| WCM160 | 8 | 163470 | 38959498 | −0.1024 | −0.8064 | 0.5951 | 0.5485 | 1 | 1 |
| WCM160 | 8 | 38961086 | 146279383 | 0.2277 | 1.0487 | 2.0687 | 2.0687 | 2 | 2 |
| WCM160 | 9 | 14886 | 141107539 | 0.0753 | 0.4169 | 1.335 | 1.335 | 1 | 1 |
| WCM160 | X | 2700189 | 65819567 | 0.0209 | 0.126 | NA | NA | NA | NA |
| WCM160 | X | 65819850 | 67454319 | 2.481 | 4.8896 | NA | NA | NA | NA |
| WCM160 | X | 67494544 | 68383243 | 1.1119 | 3.0458 | NA | NA | NA | NA |
| WCM160 | X | 68394313 | 154493601 | 0.0326 | 0.1926 | NA | NA | NA | NA |
| WCM160_X1_1 | 1 | 721450 | 249212243 | 0.0021 | NA | NA | NA | NA | NA |
| WCM160_X1_1 | 10 | 255851 | 135440080 | −0.0049 | NA | NA | NA | NA | NA |
| WCM160_X1_1 | 11 | 193112 | 134856603 | 0.0096 | NA | NA | NA | NA | NA |
| WCM160_X1_1 | 12 | 148674 | 133810703 | 0.001 | NA | NA | NA | NA | NA |
| WCM160_X1_1 | 13 | 19600475 | 115089411 | −0.0188 | NA | NA | NA | NA | NA |
| WCM160_X1_1 | 14 | 20201693 | 107283026 | 0.0059 | NA | NA | NA | NA | NA |
| WCM160_X1_1 | 15 | 20169971 | 102515282 | 6.00E−04 | NA | NA | NA | NA | NA |
| WCM160_X1_1 | 16 | 67604 | 90161743 | 0.0082 | NA | NA | NA | NA | NA |
| WCM160_X1_1 | 17 | 131595 | 81083455 | 0.0148 | NA | NA | NA | NA | NA |
| WCM160_X1_1 | 18 | 49034 | 78005160 | −0.0228 | NA | NA | NA | NA | NA |
| WCM160_X1_1 | 19 | 281430 | 59083953 | 0.0035 | NA | NA | NA | NA | NA |
| WCM160_X1_1 | 2 | 41613 | 242946604 | −7.00E−04 | NA | NA | NA | NA | NA |
| WCM160_X1_1 | 20 | 68375 | 62904835 | 0.0101 | NA | NA | NA | NA | NA |
| WCM160_X1_1 | 21 | 9826203 | 48084281 | 0.0072 | NA | NA | NA | NA | NA |
| WCM160_X1_1 | 22 | 16287454 | 51215130 | 0.0248 | NA | NA | NA | NA | NA |
| WCM160_X1_1 | 3 | 239350 | 197894713 | 0.0025 | NA | NA | NA | NA | NA |
| WCM160_X1_1 | 4 | 59331 | 190426502 | −0.0143 | NA | NA | NA | NA | NA |
| WCM160_X1_1 | 5 | 140448 | 180687768 | −0.0026 | NA | NA | NA | NA | NA |
| WCM160_X1_1 | 6 | 203615 | 170892697 | −0.0099 | NA | NA | NA | NA | NA |
| WCM160_X1_1 | 7 | 193451 | 158937022 | −0.0098 | NA | NA | NA | NA | NA |
| WCM160_X1_1 | 8 | 190906 | 146279472 | −0.0101 | NA | NA | NA | NA | NA |
| WCM160_X1_1 | 9 | 17073 | 141111414 | 0.0042 | NA | NA | NA | NA | NA |
| WCM160_X1_1 | X | 2707694 | 154509348 | −0.0526 | NA | NA | NA | NA | NA |
| WCM160_Z7_1 | 1 | 721415 | 21585265 | −0.1959 | −0.6063 | 0.9825 | 0.3312 | 1 | 0 |
| WCM160_Z7_1 | 1 | 21586773 | 62483618 | −0.1094 | −0.317 | 0.8453 | 0.7601 | 1 | 1 |
| WCM160_Z7_1 | 1 | 62503653 | 145109583 | −0.2269 | −0.7211 | 0.759 | 0.4542 | 1 | 0 |
| WCM160_Z7_1 | 1 | 145112485 | 145439923 | 0.0725 | 0.1882 | NA | NA | NA | NA |
| WCM160_Z7_1 | 1 | 145440294 | 156641049 | 0.5097 | 1.1012 | 2.5956 | 1.6951 | 3 | 2 |
| WCM160_Z7_1 | 1 | 156641353 | 156815487 | 0.0846 | 0.2181 | NA | NA | NA | NA |
| WCM160_Z7_1 | 1 | 156815892 | 180180665 | 0.0469 | 0.1235 | 1.6869 | 0.4919 | 2 | 0 |
| WCM160_Z7_1 | 1 | 180199714 | 223285646 | 0.2044 | 0.4971 | 1.4612 | 1.3615 | 1 | 1 |
| WCM160_Z7_1 | 1 | 223285946 | 229738236 | 0.0203 | 0.0541 | 1.104 | 0.9725 | 1 | 1 |
| WCM160_Z7_1 | 1 | 229738549 | 234586237 | −0.2143 | −0.6736 | 1.242 | 0.0119 | 1 | 0 |
| WCM160_Z7_1 | 1 | 234593444 | 249212111 | −0.0554 | −0.1549 | 0.9996 | 0.7967 | 1 | 1 |
| WCM160_Z7_1 | 10 | 255922 | 3167355 | −0.1738 | −0.5286 | NA | NA | NA | NA |
| WCM160_Z7_1 | 10 | 3172121 | 58117891 | −0.0776 | −0.2202 | 0.9122 | 0.8047 | 1 | 1 |
| WCM160_Z7_1 | 10 | 58118330 | 101689378 | 0.0578 | 0.1512 | 1.1302 | 1.0908 | 1 | 1 |
| WCM160_Z7_1 | 10 | 101689826 | 106075786 | −0.1185 | −0.3458 | 0.8591 | 0.7147 | 1 | 1 |
| WCM160_Z7_1 | 10 | 106113656 | 135440086 | −0.26 | −0.8513 | 1.087 | 0.0216 | 1 | 0 |
| WCM160_Z7_1 | 11 | 193112 | 65167238 | 0.0478 | 0.1257 | 1.1134 | 1.0687 | 1 | 1 |
| WCM160_Z7_1 | 11 | 65168230 | 105481795 | 0.1691 | 0.4181 | 1.3696 | 1.3028 | 1 | 1 |
| WCM160_Z7_1 | 11 | 105483133 | 107326440 | 0.0939 | 0.2409 | NA | NA | NA | NA |
| WCM160_Z7_1 | 11 | 107328498 | 126162862 | −0.2482 | −0.804 | 1.0961 | 0.0494 | 1 | 0 |
| WCM160_Z7_1 | 11 | 126162885 | 134856489 | 0.0269 | 0.0716 | 1.1611 | 0.9408 | 1 | 1 |
| WCM160_Z7_1 | 12 | 148675 | 133810703 | −0.0903 | −0.2585 | 1.1736 | 0.4983 | 1 | 0 |
| WCM160_Z7_1 | 13 | 19600475 | 19997282 | −0.213 | −0.669 | NA | NA | NA | NA |
| WCM160_Z7_1 | 13 | 20012234 | 32676149 | 0.0378 | 0.1 | 1.4408 | 0.7028 | 1 | 1 |
| WCM160_Z7_1 | 13 | 32691547 | 115089411 | −0.2384 | −0.7652 | 0.6365 | 0.5403 | 1 | 1 |
| WCM160_Z7_1 | 14 | 20201708 | 20470903 | −0.1446 | −0.4301 | 1.5955 | −0.1111 | 2 | 0 |
| WCM160_Z7_1 | 14 | 20471089 | 38679473 | 0.05 | 0.1314 | 1.1038 | 1.087 | 1 | 1 |
| WCM160_Z7_1 | 14 | 38679656 | 107283027 | −0.0919 | −0.2634 | 1.107 | 0.5593 | 1 | 1 |
| WCM160_Z7_1 | 15 | 20169972 | 20876515 | 0.2536 | 0.6036 | 2.2348 | 0.8041 | 2 | 1 |
| WCM160_Z7_1 | 15 | 21145647 | 37100525 | 0.1579 | 0.3924 | 1.7347 | 0.8904 | 2 | 1 |
| WCM160_Z7_1 | 15 | 37101104 | 102515271 | −0.0895 | −0.2561 | 1.1142 | 0.5605 | 1 | 1 |
| WCM160_Z7_1 | 16 | 67604 | 18847528 | 0.3002 | 0.7005 | 2.1542 | 1.096 | 2 | 1 |
| WCM160_Z7_1 | 16 | 18847837 | 30569350 | 0.5463 | 1.1662 | 3.5545 | 0.9338 | 4 | 1 |
| WCM160_Z7_1 | 16 | 30581403 | 30719006 | 0.1591 | 0.3951 | NA | NA | NA | NA |
| WCM160_Z7_1 | 16 | 30721055 | 61687709 | −0.0482 | −0.134 | 1.2276 | 0.595 | 1 | 1 |
| WCM160_Z7_1 | 16 | 61687979 | 90108923 | −0.2553 | −0.8322 | 1.1323 | −0.009 | 1 | 0 |
| WCM160_Z7_1 | 16 | 90109702 | 90161547 | −0.0985 | −0.2835 | NA | NA | NA | NA |
| WCM160_Z7_1 | 17 | 131595 | 10101706 | −0.2669 | −0.8798 | 1.0398 | 0.047 | 1 | 0 |
| WCM160_Z7_1 | 17 | 10204987 | 21318882 | 0.0865 | 0.2227 | 1.2014 | 1.1325 | 1 | 1 |
| WCM160_Z7_1 | 17 | 21318996 | 29761098 | 0.2187 | 0.5284 | 1.7474 | 1.1372 | 2 | 1 |
| WCM160_Z7_1 | 17 | 29844704 | 37899218 | 0.5951 | 1.2509 | 3.7393 | 1.0203 | 4 | 1 |
| WCM160_Z7_1 | 17 | 37899479 | 81083419 | −0.1044 | −0.3018 | 1.1456 | 0.4769 | 1 | 0 |
| WCM160_Z7_1 | 18 | 49034 | 78005206 | −0.119 | −0.3474 | 0.9428 | 0.6292 | 1 | 1 |
| WCM160_Z7_1 | 19 | 281507 | 59083988 | −0.1119 | −0.3249 | 1.131 | 0.4657 | 1 | 0 |
| WCM160_Z7_1 | 2 | 41612 | 1457570 | −0.0159 | −0.0435 | NA | NA | NA | NA |
| WCM160_Z7_1 | 2 | 1459965 | 26534054 | −0.0831 | −0.2366 | 1.6372 | 0.0604 | 2 | 0 |
| WCM160_Z7_1 | 2 | 26534322 | 26534345 | −2.4875 | NA | NA | NA | NA | NA |
| WCM160_Z7_1 | 2 | 26534813 | 71886073 | −0.0755 | −0.214 | 1.7136 | 0.0106 | 2 | 0 |
| WCM160_Z7_1 | 2 | 71887715 | 89890579 | −0.2628 | −0.8627 | 1.0951 | 0.0048 | 1 | 0 |
| WCM160_Z7_1 | 2 | 89986379 | 90139116 | 0.5343 | 1.145 | NA | NA | NA | NA |
| WCM160_Z7_1 | 2 | 90139370 | 202260154 | −0.0694 | −0.196 | 1.0996 | 0.6463 | 1 | 1 |
| WCM160_Z7_1 | 2 | 202262942 | 234449316 | 0.0744 | 0.1928 | 1.6809 | 0.6052 | 2 | 1 |
| WCM160_Z7_1 | 2 | 234450931 | 242946607 | −0.1187 | −0.3466 | 0.8288 | 0.7441 | 1 | 1 |
| WCM160_Z7_1 | 20 | 68396 | 55012318 | 0.0357 | 0.0946 | 1.0872 | 1.0484 | 1 | 1 |
| WCM160_Z7_1 | 20 | 55012582 | 62904835 | 0.3256 | 0.7521 | 2.0669 | 1.3015 | 2 | 1 |
| WCM160_Z7_1 | 21 | 9826205 | 48084282 | −0.2764 | −0.9194 | 0.5578 | 0.4997 | 1 | 0 |
| WCM160_Z7_1 | 22 | 16287431 | 46486972 | −0.1084 | −0.3142 | 1.0726 | 0.5361 | 1 | 1 |
| WCM160_Z7_1 | 22 | 46493816 | 51215153 | −0.4762 | −2.0579 | 0.485 | −0.0046 | 0 | 0 |
| WCM160_Z7_1 | 3 | 239531 | 197894713 | −0.0809 | −0.2301 | 1.1183 | 0.5868 | 1 | 1 |
| WCM160_Z7_1 | 4 | 59334 | 47034626 | −0.0885 | −0.2528 | 1.1062 | 0.5723 | 1 | 1 |
| WCM160_Z7_1 | 4 | 47034738 | 57676321 | 0.1494 | 0.3728 | 1.5227 | 1.0671 | 2 | 1 |
| WCM160_Z7_1 | 4 | 57686706 | 75107314 | −0.0872 | −0.2489 | 1.6608 | 0.0223 | 2 | 0 |
| WCM160_Z7_1 | 4 | 75147195 | 190426502 | 0.0993 | 0.2541 | 1.6732 | 0.7119 | 2 | 1 |
| WCM160_Z7_1 | 5 | 140477 | 162910046 | −0.0762 | −0.216 | 1.1352 | 0.5866 | 1 | 1 |
| WCM160_Z7_1 | 5 | 162930418 | 180687768 | 0.178 | 0.4382 | 1.4405 | 1.2693 | 1 | 1 |
| WCM160_Z7_1 | 6 | 203624 | 398901 | −0.1096 | −0.3177 | NA | NA | NA | NA |
| WCM160_Z7_1 | 6 | 401492 | 64791847 | 0.1827 | 0.4487 | 1.6869 | 1.0427 | 2 | 1 |
| WCM160_Z7_1 | 6 | 64940499 | 170892612 | −0.0851 | −0.2428 | 1.1534 | 0.5368 | 1 | 1 |
| WCM160_Z7_1 | 7 | 193254 | 158937227 | −0.1088 | −0.3152 | 0.8288 | 0.7787 | 1 | 1 |
| WCM160_Z7_1 | 8 | 190907 | 39550165 | −0.3175 | −1.1014 | 0.7675 | 0.1646 | 1 | 0 |
| WCM160_Z7_1 | 8 | 39581427 | 146279498 | 0.3163 | 0.7333 | 2.2144 | 1.1106 | 2 | 1 |
| WCM160_Z7_1 | 9 | 17107 | 141111414 | −0.0069 | −0.0187 | 1.014 | 0.9602 | 1 | 1 |
| WCM160_Z7_1 | X | 2707709 | 65474878 | 0.0038 | 0.0101 | NA | NA | NA | NA |
| WCM160_Z7_1 | X | 65476180 | 68382719 | 1.7329 | 2.8641 | NA | NA | NA | NA |
| WCM160_Z7_1 | X | 68382969 | 154509348 | 0.0144 | 0.0387 | NA | NA | NA | NA |
| WCM161 | 1 | 14930 | 35470728 | −0.0226 | −0.0169 | 0.9884 | 0.9884 | 1 | 1 |
| WCM161 | 1 | 35472611 | 51323474 | −0.1413 | −0.2612 | 0.8344 | 0.8344 | 1 | 1 |
| WCM161 | 1 | 51425515 | 111397084 | −0.3116 | −0.6563 | 0.7844 | 0.4845 | 1 | 0 |
| WCM161 | 1 | 111397401 | 118426295 | −0.207 | −0.4065 | 0.7544 | 0.7544 | 1 | 1 |
| WCM161 | 1 | 118429221 | 167367336 | 0.1167 | 0.2461 | 1.186 | 1.186 | 1 | 1 |
| WCM161 | 1 | 167368543 | 178269220 | −0.2422 | −0.4877 | 1.2103 | 0.216 | 1 | 0 |
| WCM161 | 1 | 178271298 | 249214028 | 0.1348 | 0.2787 | 1.2131 | 1.2131 | 1 | 1 |
| WCM161 | 10 | 225958 | 459820 | −0.0949 | −0.1632 | NA | NA | NA | NA |
| WCM161 | 10 | 460082 | 11312806 | −0.2288 | −0.4565 | 1.1224 | 0.3351 | 1 | 0 |
| WCM161 | 10 | 11317093 | 15885306 | 0.1103 | 0.2343 | 1.2234 | 1.1293 | 1 | 1 |
| WCM161 | 10 | 15889863 | 21807083 | −0.3096 | −0.6512 | 1.1463 | 0.1271 | 1 | 0 |
| WCM161 | 10 | 21807340 | 28599927 | −0.0859 | −0.1446 | 0.9699 | 0.8393 | 1 | 1 |
| WCM161 | 10 | 28821811 | 29844067 | −0.4682 | −1.088 | 0.9384 | 0.0024 | 1 | 0 |
| WCM161 | 10 | 29852240 | 38622117 | −0.1351 | −0.2478 | 0.8963 | 0.7881 | 1 | 1 |
| WCM161 | 10 | 38633714 | 38679824 | −0.3068 | −0.6443 | NA | NA | NA | NA |
| WCM161 | 10 | 38680963 | 135477897 | 0.0199 | 0.0657 | 1.0466 | 1.0466 | 1 | 1 |
| WCM161 | 11 | 193096 | 1269642 | 0.12 | 0.2521 | 1.7611 | 0.6207 | 2 | 1 |
| WCM161 | 11 | 1270348 | 26725462 | 0.0682 | 0.1569 | 1.7231 | 0.5067 | 2 | 1 |
| WCM161 | 11 | 26730894 | 65234014 | 0.0728 | 0.1656 | 1.6006 | 0.6427 | 2 | 1 |
| WCM161 | 11 | 65265357 | 65293819 | −1.2204 | NA | NA | NA | NA | NA |
| WCM161 | 11 | 65294444 | 94209605 | 0.0865 | 0.1909 | 1.5556 | 0.7273 | 2 | 1 |
| WCM161 | 11 | 94211871 | 134241094 | −0.1056 | −0.1854 | 0.8978 | 0.861 | 1 | 1 |
| WCM161 | 11 | 134241320 | 134605849 | 0.0102 | 0.0472 | NA | NA | NA | NA |
| WCM161 | 12 | 148485 | 11547405 | −0.1797 | −0.3451 | 0.789 | 0.7855 | 1 | 1 |
| WCM161 | 12 | 11548341 | 20602432 | −0.4754 | −1.1101 | 0.8179 | 0.1087 | 1 | 0 |
| WCM161 | 12 | 20704634 | 21317791 | −0.0257 | −0.0231 | 1.0366 | 0.9316 | 1 | 1 |
| WCM161 | 12 | 21319526 | 30906509 | −0.4547 | −1.0474 | 0.7829 | 0.1848 | 1 | 0 |
| WCM161 | 12 | 31106915 | 100043645 | −0.0064 | 0.0148 | 1.0103 | 1.0103 | 1 | 1 |
| WCM161 | 12 | 100048854 | 133810880 | −0.1011 | −0.176 | 0.8852 | 0.8852 | 1 | 1 |
| WCM161 | 13 | 19042004 | 22141055 | 0.7439 | 1.2357 | 3.4989 | 1.211 | 3 | 1 |
| WCM161 | 13 | 22178125 | 37453797 | −0.3637 | −0.7909 | 1.0073 | 0.1487 | 1 | 0 |
| WCM161 | 13 | 37524255 | 40362491 | 0.6493 | 1.1007 | 3.2595 | 1.0298 | 3 | 1 |
| WCM161 | 13 | 40783682 | 42897300 | −0.4059 | −0.9063 | 0.9101 | 0.157 | 1 | 0 |
| WCM161 | 13 | 43148586 | 46594713 | 0.5942 | 1.0202 | 3.0415 | 1.015 | 3 | 1 |
| WCM161 | 13 | 46616324 | 58299838 | −0.5909 | −1.4992 | 0.5628 | 0.1447 | 1 | 0 |
| WCM161 | 13 | 58300293 | 95271496 | −0.1654 | −0.3135 | 0.8082 | 0.8012 | 1 | 1 |
| WCM161 | 13 | 95271797 | 101315270 | −0.5872 | −1.4856 | 0.5218 | 0.1924 | 1 | 0 |
| WCM161 | 13 | 101315464 | 115089411 | 0.0505 | 0.1238 | 1.2967 | 0.8825 | 1 | 1 |
| WCM161 | 14 | 19110348 | 51722752 | 0.1292 | 0.2686 | 1.2047 | 1.2047 | 1 | 1 |
| WCM161 | 14 | 51956133 | 82000125 | −0.118 | −0.2115 | 0.9239 | 0.8034 | 1 | 1 |
| WCM161 | 14 | 82127872 | 107283255 | 0.1242 | 0.2595 | 1.1971 | 1.1971 | 1 | 1 |
| WCM161 | 15 | 20083992 | 78274014 | −0.0735 | −0.1191 | 1.2143 | 0.6272 | 1 | 1 |
| WCM161 | 15 | 78277960 | 102506273 | 0.0489 | 0.1209 | 1.0956 | 1.0792 | 1 | 1 |
| WCM161 | 16 | 66905 | 72816877 | −0.0107 | 0.0064 | 1.0045 | 1.0045 | 1 | 1 |
| WCM161 | 16 | 72817159 | 90260461 | −0.441 | −1.0067 | 0.8229 | 0.1724 | 1 | 0 |
| WCM161 | 17 | 131595 | 21176028 | −0.2256 | −0.449 | 1.3726 | 0.0925 | 1 | 0 |
| WCM161 | 17 | 21201719 | 77769837 | 0.1906 | 0.3771 | 1.2988 | 1.2988 | 1 | 1 |
| WCM161 | 17 | 77770234 | 80858575 | 0.0305 | 0.086 | 1.381 | 0.7419 | 1 | 1 |
| WCM161 | 17 | 80861313 | 81187946 | 0.2563 | 0.4895 | NA | NA | NA | NA |
| WCM161 | 18 | 47692 | 14157278 | 0.3543 | 0.651 | 1.8309 | 1.3095 | 2 | 1 |
| WCM161 | 18 | 14169508 | 44057760 | 0.1848 | 0.367 | 1.3167 | 1.2627 | 1 | 1 |
| WCM161 | 18 | 44063639 | 46388898 | 0.3263 | 0.6056 | 1.6358 | 1.4074 | 2 | 1 |
| WCM161 | 18 | 46446442 | 78005215 | −0.0158 | −0.0035 | 0.9976 | 0.9976 | 1 | 1 |
| WCM161 | 19 | 105080 | 9251120 | −0.1507 | −0.2815 | 0.8388 | 0.8067 | 1 | 1 |
| WCM161 | 19 | 9266682 | 34583879 | 0.0759 | 0.1713 | 1.1449 | 1.1073 | 1 | 1 |
| WCM161 | 19 | 34583985 | 59093696 | −0.2276 | −0.4537 | 0.7302 | 0.7302 | 1 | 1 |
| WCM161 | 2 | 41463 | 206628638 | −0.086 | −0.1447 | 0.9046 | 0.9046 | 1 | 1 |
| WCM161 | 2 | 206630337 | 240500567 | −0.1822 | −0.3506 | 0.7843 | 0.7843 | 1 | 1 |
| WCM161 | 2 | 240504813 | 243037116 | −0.105 | −0.1841 | 0.8894 | 0.871 | 1 | 1 |
| WCM161 | 20 | 68397 | 62934592 | 0.1761 | 0.3518 | 1.2762 | 1.2762 | 1 | 1 |
| WCM161 | 21 | 9590301 | 15310168 | −0.0643 | −0.1003 | 1.2586 | 0.6071 | 1 | 1 |
| WCM161 | 21 | 15310356 | 22840930 | −0.0957 | −0.1647 | 1.6129 | 0.1713 | 2 | 0 |
| WCM161 | 21 | 22841154 | 39754989 | −0.2411 | −0.4853 | 1.0678 | 0.3609 | 1 | 0 |
| WCM161 | 21 | 39755310 | 42866347 | 0.1164 | 0.2455 | 2.0332 | 0.3377 | 2 | 0 |
| WCM161 | 21 | 42866514 | 48084114 | −0.136 | −0.2498 | 1.2933 | 0.3888 | 1 | 0 |
| WCM161 | 22 | 16159046 | 38531080 | −0.0539 | −0.0792 | 1.2441 | 0.649 | 1 | 1 |
| WCM161 | 22 | 38536000 | 51045433 | −0.1827 | −0.3518 | 0.8377 | 0.7295 | 1 | 1 |
| WCM161 | 22 | 51048059 | 51223730 | −0.1369 | −0.2517 | 1.0434 | 0.6364 | 1 | 1 |
| WCM161 | 3 | 239339 | 49457771 | −0.0814 | −0.1353 | 1.266 | 0.555 | 1 | 1 |
| WCM161 | 3 | 49457846 | 197445074 | −0.023 | −0.0177 | 0.9878 | 0.9878 | 1 | 1 |
| WCM161 | 3 | 197476474 | 197896798 | −0.2063 | −0.4049 | NA | NA | NA | NA |
| WCM161 | 4 | 53397 | 46929505 | −0.0391 | −0.0496 | 1.1628 | 0.7697 | 1 | 1 |
| WCM161 | 4 | 46929587 | 61529532 | 0.1588 | 0.3214 | 1.2495 | 1.2495 | 1 | 1 |
| WCM161 | 4 | 61529897 | 77305346 | 0.405 | 0.7319 | 2.1012 | 1.2204 | 2 | 1 |
| WCM161 | 4 | 77305572 | 89591376 | 0.1641 | 0.3308 | 1.3486 | 1.1667 | 1 | 1 |
| WCM161 | 4 | 89597416 | 119613045 | 0.0189 | 0.0638 | 1.0452 | 1.0452 | 1 | 1 |
| WCM161 | 4 | 119615699 | 128584736 | 0.2577 | 0.492 | 1.8183 | 0.9945 | 2 | 1 |
| WCM161 | 4 | 128590255 | 139988840 | −0.016 | −0.0039 | 1.0404 | 0.9542 | 1 | 1 |
| WCM161 | 4 | 139992984 | 177095753 | 0.2095 | 0.4099 | 1.6399 | 1.0172 | 2 | 1 |
| WCM161 | 4 | 177098348 | 190904482 | −0.1534 | −0.2873 | 0.8444 | 0.7945 | 1 | 1 |
| WCM161 | 5 | 90647 | 180687773 | −0.0057 | 0.0161 | 1.0113 | 1.0113 | 1 | 1 |
| WCM161 | 6 | 203475 | 70386431 | −0.0924 | −0.1581 | 1.0615 | 0.731 | 1 | 1 |
| WCM161 | 6 | 70407434 | 170892918 | −0.2223 | −0.4414 | 0.7364 | 0.7364 | 1 | 1 |
| WCM161 | 7 | 193185 | 48335533 | 0.174 | 0.3481 | 1.2729 | 1.2729 | 1 | 1 |
| WCM161 | 7 | 48336844 | 80088200 | −0.1832 | −0.3529 | 1.2974 | 0.2686 | 1 | 0 |
| WCM161 | 7 | 80091509 | 104485901 | 0.162 | 0.327 | 1.456 | 1.0528 | 1 | 1 |
| WCM161 | 7 | 104546608 | 158937158 | −0.1643 | −0.3111 | 1.481 | 0.1311 | 1 | 0 |
| WCM161 | 8 | 163457 | 32621932 | −0.4452 | −1.0192 | 0.8278 | 0.1589 | 1 | 0 |
| WCM161 | 8 | 33230203 | 41575226 | 0.7931 | 1.3046 | 4.0586 | 0.8816 | 4 | 1 |
| WCM161 | 8 | 41575637 | 85249809 | 0.6851 | 1.1522 | 3.445 | 1.0002 | 3 | 1 |
| WCM161 | 8 | 85272976 | 128959818 | 0.8493 | 1.382 | 4.1616 | 1.0511 | 4 | 1 |
| WCM161 | 8 | 128960272 | 145678720 | 1.0767 | 1.685 | 4.915 | 1.5156 | 5 | 2 |
| WCM161 | 8 | 145689642 | 146279527 | 0.8721 | 1.4131 | NA | NA | NA | NA |
| WCM161 | 9 | 14857 | 141107515 | 0.1939 | 0.3829 | 1.304 | 1.304 | 1 | 1 |
| WCM161 | X | 2700147 | 7870115 | −0.2311 | −0.4619 | NA | NA | NA | NA |
| WCM161 | X | 7880072 | 9935585 | 0.9088 | 1.4628 | NA | NA | NA | NA |
| WCM161 | X | 9935807 | 14937969 | 0.1656 | 0.3334 | NA | NA | NA | NA |
| WCM161 | X | 14938390 | 64754607 | 0.4917 | 0.8664 | NA | NA | NA | NA |
| WCM161 | X | 64887746 | 67518904 | 1.4727 | 2.1809 | NA | NA | NA | NA |
| WCM161 | X | 67652875 | 67942444 | 0.3003 | 0.5629 | NA | NA | NA | NA |
| WCM161 | X | 67943988 | 154509348 | 0.1644 | 0.3313 | NA | NA | NA | NA |
| WCM161_Z1_1 | 1 | 69640 | 40363562 | −0.258 | 0.5253 | 1.9011 | 0.9772 | 2 | 1 |
| WCM161_Z1_1 | 1 | 40366619 | 111217266 | −0.7153 | −0.0698 | 1.9008 | 0.0047 | 2 | 0 |
| WCM161_Z1_1 | 1 | 111434052 | 145209388 | −0.3028 | 0.4693 | 1.9139 | 0.855 | 2 | 1 |
| WCM161_Z1_1 | 1 | 145281656 | 167358840 | 0.3399 | 1.238 | 2.8411 | 1.8763 | 3 | 2 |
| WCM161_Z1_1 | 1 | 167365520 | 171765830 | −0.7456 | −0.1114 | 1.8911 | −0.0398 | 2 | 0 |
| WCM161_Z1_1 | 1 | 171765886 | 174992477 | −0.2728 | 0.5068 | 1.9278 | 0.9141 | 2 | 1 |
| WCM161_Z1_1 | 1 | 175037040 | 178252706 | −0.7414 | −0.1056 | 1.8888 | −0.0299 | 2 | 0 |
| WCM161_Z1_1 | 1 | 178269164 | 249212557 | 0.0454 | 0.8939 | 1.8582 | 1.8582 | 2 | 2 |
| WCM161_Z1_1 | 10 | 225979 | 5141556 | −0.2997 | 0.4732 | NA | NA | NA | NA |
| WCM161_Z1_1 | 10 | 5144319 | 9281413 | −0.6732 | −0.0125 | 1.9581 | 0.0246 | 2 | 0 |
| WCM161_Z1_1 | 10 | 9449213 | 15902209 | −0.0094 | 0.8286 | 1.8143 | 1.7377 | 2 | 2 |
| WCM161_Z1_1 | 10 | 16526423 | 21461323 | −0.7472 | −0.1136 | 1.8542 | −0.0057 | 2 | 0 |
| WCM161_Z1_1 | 10 | 21784753 | 28438913 | −0.2655 | 0.516 | 1.5564 | 1.3035 | 2 | 1 |
| WCM161_Z1_1 | 10 | 28491144 | 30306828 | −0.7209 | −0.0775 | 1.8844 | 0.0111 | 2 | 0 |
| WCM161_Z1_1 | 10 | 30315095 | 39147119 | −0.2899 | 0.4855 | 1.88 | 0.9202 | 2 | 1 |
| WCM161_Z1_1 | 10 | 42905606 | 44053435 | 0.0401 | 0.8877 | NA | NA | NA | NA |
| WCM161_Z1_1 | 10 | 44102500 | 75868764 | 0.0686 | 0.9215 | 1.9098 | 1.8784 | 2 | 2 |
| WCM161_Z1_1 | 10 | 75871762 | 96454758 | 0.3331 | 1.2302 | 2.7506 | 1.9414 | 3 | 2 |
| WCM161_Z1_1 | 10 | 96466623 | 96609749 | 0.4115 | 1.32 | NA | NA | NA | NA |
| WCM161_Z1_1 | 10 | 96612495 | 135440090 | 0.0434 | 0.8915 | 2.7554 | 0.9549 | 3 | 1 |
| WCM161_Z1_1 | 11 | 193105 | 6412129 | 0.0595 | 0.9107 | 2.7501 | 1.0097 | 3 | 1 |
| WCM161_Z1_1 | 11 | 6412744 | 7092474 | 0.3703 | 1.2728 | 2.6128 | 2.2198 | 3 | 2 |
| WCM161_Z1_1 | 11 | 7110548 | 30034151 | 0.039 | 0.8864 | 2.7483 | 0.9488 | 3 | 1 |
| WCM161_Z1_1 | 11 | 30255185 | 96125013 | 0.3393 | 1.2373 | 2.8641 | 1.851 | 3 | 2 |
| WCM161_Z1_1 | 11 | 96125317 | 113320759 | 0.0116 | 0.8537 | 1.9085 | 1.7056 | 2 | 2 |
| WCM161_Z1_1 | 11 | 113558990 | 134856604 | 0.3578 | 1.2585 | 2.8205 | 1.9645 | 3 | 2 |
| WCM161_Z1_1 | 12 | 148675 | 11903775 | −0.709 | −0.0611 | 0.9834 | 0.9336 | 1 | 1 |
| WCM161_Z1_1 | 12 | 11905386 | 20522733 | −1.3283 | −0.9961 | 0.9448 | 0.0579 | 1 | 0 |
| WCM161_Z1_1 | 12 | 20523039 | 21325668 | −0.2658 | 0.5156 | NA | NA | NA | NA |
| WCM161_Z1_1 | 12 | 21327644 | 31079921 | −1.3577 | −1.0464 | 0.993 | −0.0247 | 1 | 0 |
| WCM161_Z1_1 | 12 | 31106968 | 133810712 | −0.2641 | 0.5177 | 1.8524 | 1.0109 | 2 | 1 |
| WCM161_Z1_1 | 13 | 19600515 | 22178262 | 1.4418 | 2.4503 | NA | NA | NA | NA |
| WCM161_Z1_1 | 13 | 22246243 | 26828877 | −1.3502 | −1.0335 | 0.9874 | −0.0104 | 1 | 0 |
| WCM161_Z1_1 | 13 | 26923262 | 29608109 | 0.2477 | 1.1315 | NA | NA | NA | NA |
| WCM161_Z1_1 | 13 | 29675095 | 30091439 | −1.0915 | −0.6138 | NA | NA | NA | NA |
| WCM161_Z1_1 | 13 | 30091835 | 32932032 | 0.2618 | 1.1479 | NA | NA | NA | NA |
| WCM161_Z1_1 | 13 | 32936801 | 37453698 | −1.1016 | −0.6294 | 1.3159 | −0.0231 | 1 | 0 |
| WCM161_Z1_1 | 13 | 37524175 | 40362472 | 1.4669 | 2.477 | NA | NA | NA | NA |
| WCM161_Z1_1 | 13 | 41133727 | 42893286 | −1.3827 | −1.09 | NA | NA | NA | NA |
| WCM161_Z1_1 | 13 | 43148655 | 46594663 | 1.5015 | 2.5138 | NA | NA | NA | NA |
| WCM161_Z1_1 | 13 | 46616338 | 58299377 | −1.3132 | −0.9705 | 1.0902 | −0.0696 | 1 | 0 |
| WCM161_Z1_1 | 13 | 60240961 | 61141739 | −0.1884 | 0.6114 | NA | NA | NA | NA |
| WCM161_Z1_1 | 13 | 61985607 | 95257649 | −0.7593 | −0.1304 | 1.0806 | 0.7466 | 1 | 1 |
| WCM161_Z1_1 | 13 | 95264604 | 101308657 | −1.3898 | −1.1023 | 0.9315 | 0 | 1 | 0 |
| WCM161_Z1_1 | 13 | 101315395 | 115089411 | −0.0989 | 0.7207 | 2.2998 | 0.9962 | 2 | 1 |
| WCM161_Z1_1 | 14 | 19377716 | 21699145 | 0.0602 | 0.9115 | 2.8588 | 0.9032 | 3 | 1 |
| WCM161_Z1_1 | 14 | 21702176 | 50998701 | 0.3361 | 1.2336 | 2.7597 | 1.9434 | 3 | 2 |
| WCM161_Z1_1 | 14 | 51027003 | 51721117 | −0.3155 | 0.4534 | NA | NA | NA | NA |
| WCM161_Z1_1 | 14 | 52156572 | 53619818 | −1.4031 | −1.1258 | NA | NA | NA | NA |
| WCM161_Z1_1 | 14 | 54416901 | 87379886 | −0.2738 | 0.5056 | 2.8401 | −7.00E−04 | 3 | 0 |
| WCM161_Z1_1 | 14 | 87386660 | 93397704 | 0.2949 | 1.1862 | 2.4331 | 2.1179 | 2 | 2 |
| WCM161_Z1_1 | 14 | 93398046 | 107283027 | 0.7369 | 1.6857 | 3.267 | 3.167 | 3 | 3 |
| WCM161_Z1_1 | 15 | 20170113 | 102506284 | −0.7111 | −0.064 | 1.0456 | 0.8677 | 1 | 1 |
| WCM161_Z1_1 | 16 | 67604 | 65025718 | 0.0842 | 0.94 | 1.9412 | 1.8958 | 2 | 2 |
| WCM161_Z1_1 | 16 | 65026863 | 72198696 | −0.2318 | 0.5578 | 1.9115 | 1.0326 | 2 | 1 |
| WCM161_Z1_1 | 16 | 72821129 | 90233626 | −0.6908 | −0.0363 | 1.9501 | 2.00E−04 | 2 | 0 |
| WCM161_Z1_1 | 17 | 131595 | 21146674 | −0.2721 | 0.5077 | 2.8332 | 0.0104 | 3 | 0 |
| WCM161_Z1_1 | 17 | 21147526 | 68172454 | 0.0719 | 0.9254 | 1.9262 | 1.8722 | 2 | 2 |
| WCM161_Z1_1 | 17 | 70117582 | 74729698 | 0.2879 | 1.178 | 2.3149 | 2.2104 | 2 | 2 |
| WCM161_Z1_1 | 17 | 74732285 | 74936537 | −0.0792 | 0.7446 | NA | NA | NA | NA |
| WCM161_Z1_1 | 17 | 74936836 | 81083398 | −0.2325 | 0.5569 | 1.9723 | 0.9699 | 2 | 1 |
| WCM161_Z1_1 | 18 | 48339 | 31226226 | 0.2748 | 1.1629 | 2.5292 | 1.949 | 3 | 2 |
| WCM161_Z1_1 | 18 | 31241685 | 77891027 | −0.0151 | 0.8217 | 2.6129 | 0.9221 | 3 | 1 |
| WCM161_Z1_1 | 18 | 77893683 | 78005224 | 0.0155 | 0.8583 | NA | NA | NA | NA |
| WCM161_Z1_1 | 19 | 281498 | 19379441 | −0.2765 | 0.5023 | 1.922 | 0.9109 | 2 | 1 |
| WCM161_Z1_1 | 19 | 19380538 | 59083953 | −0.7116 | −0.0647 | 1.096 | 0.8162 | 1 | 1 |
| WCM161_Z1_1 | 2 | 41609 | 242946597 | −0.2834 | 0.4936 | 1.8619 | 0.9541 | 2 | 1 |
| WCM161_Z1_1 | 20 | 68373 | 31238927 | 0.071 | 0.9244 | 1.9203 | 1.8753 | 2 | 2 |
| WCM161_Z1_1 | 20 | 31291216 | 31998055 | −0.2876 | 0.4884 | NA | NA | NA | NA |
| WCM161_Z1_1 | 20 | 32000152 | 62904845 | 0.0878 | 0.9443 | 1.9447 | 1.9038 | 2 | 2 |
| WCM161_Z1_1 | 21 | 9826230 | 11097630 | 0.9211 | 1.8887 | NA | NA | NA | NA |
| WCM161_Z1_1 | 21 | 14745377 | 19169411 | −0.7534 | −0.1222 | NA | NA | NA | NA |
| WCM161_Z1_1 | 21 | 19190566 | 39822803 | −0.2584 | 0.5247 | 1.8683 | 1.0091 | 2 | 1 |
| WCM161_Z1_1 | 21 | 39947593 | 42866299 | 0.8494 | 1.8101 | 6.0143 | 0.9989 | 6 | 1 |
| WCM161_Z1_1 | 21 | 42870095 | 48084211 | −0.207 | 0.5884 | 1.9784 | 1.0287 | 2 | 1 |
| WCM161_Z1_1 | 22 | 17063431 | 19230365 | 0.4125 | 1.3211 | 2.9744 | 2.0226 | 3 | 2 |
| WCM161_Z1_1 | 22 | 19347005 | 24837301 | −0.2247 | 0.5666 | 1.963 | 0.9991 | 2 | 1 |
| WCM161_Z1_1 | 22 | 24837413 | 24951586 | 0.645 | 1.5834 | NA | NA | NA | NA |
| WCM161_Z1_1 | 22 | 24953769 | 26423602 | −0.2706 | 0.5096 | 1.9988 | 0.8486 | 2 | 1 |
| WCM161_Z1_1 | 22 | 26565683 | 29678535 | 0.5362 | 1.4614 | 3.5548 | 1.9526 | 4 | 2 |
| WCM161_Z1_1 | 22 | 29683020 | 36745230 | −0.2432 | 0.5437 | 1.9099 | 1.0054 | 2 | 1 |
| WCM161_Z1_1 | 22 | 36863959 | 37769219 | 0.8536 | 1.8146 | 5.2316 | 1.8037 | 5 | 2 |
| WCM161_Z1_1 | 22 | 37769606 | 39239439 | 1.2786 | 2.2759 | 7.8923 | 1.794 | 8 | 2 |
| WCM161_Z1_1 | 22 | 39262250 | 40814504 | −0.2211 | 0.571 | 1.9191 | 1.052 | 2 | 1 |
| WCM161_Z1_1 | 22 | 40814636 | 50020539 | −0.6682 | −0.0057 | 1.12 | 0.8721 | 1 | 1 |
| WCM161_Z1_1 | 22 | 50168042 | 50708692 | 1.2712 | 2.268 | 8.6925 | 0.9406 | 9 | 1 |
| WCM161_Z1_1 | 22 | 50713811 | 51215138 | −0.6901 | −0.0354 | 1.0262 | 0.9253 | 1 | 1 |
| WCM161_Z1_1 | 3 | 239405 | 49343264 | 0.1707 | 1.0418 | 3.1361 | 0.9814 | 3 | 1 |
| WCM161_Z1_1 | 3 | 49348116 | 49462402 | 0.2081 | 1.0854 | NA | NA | NA | NA |
| WCM161_Z1_1 | 3 | 49462825 | 99569326 | 0.408 | 1.3159 | 3.1629 | 1.8163 | 3 | 2 |
| WCM161_Z1_1 | 3 | 99569595 | 170218920 | 0.2237 | 1.1037 | 2.4165 | 1.8815 | 2 | 2 |
| WCM161_Z1_1 | 3 | 170244462 | 197259373 | 0.0243 | 0.8688 | 1.8566 | 1.7957 | 2 | 2 |
| WCM161_Z1_1 | 3 | 197260370 | 197410211 | 0.2666 | 1.1534 | NA | NA | NA | NA |
| WCM161_Z1_1 | 3 | 197411090 | 197894713 | 0.0636 | 0.9155 | NA | NA | NA | NA |
| WCM161_Z1_1 | 4 | 59334 | 8129285 | 0.1787 | 1.0511 | 3.182 | 0.9622 | 3 | 1 |
| WCM161_Z1_1 | 4 | 8206988 | 10566308 | −0.2382 | 0.5498 | 1.6018 | 1.3259 | 2 | 1 |
| WCM161_Z1_1 | 4 | 10567746 | 17782908 | 0.0924 | 0.9497 | NA | NA | NA | NA |
| WCM161_Z1_1 | 4 | 17805295 | 49032896 | −0.1946 | 0.6038 | 2.1128 | 0.9266 | 2 | 1 |
| WCM161_Z1_1 | 4 | 49034724 | 54968018 | 0.43 | 1.341 | NA | NA | NA | NA |
| WCM161_Z1_1 | 4 | 55124955 | 57976254 | −0.1617 | 0.6442 | NA | NA | NA | NA |
| WCM161_Z1_1 | 4 | 57976479 | 77357286 | 0.6962 | 1.6405 | 5.3624 | 0.8733 | 5 | 1 |
| WCM161_Z1_1 | 4 | 77476765 | 85552977 | 0.3018 | 1.1941 | 3.6815 | 0.8944 | 4 | 1 |
| WCM161_Z1_1 | 4 | 85555069 | 89052323 | 0.6436 | 1.5819 | 5.1513 | 0.8361 | 5 | 1 |
| WCM161_Z1_1 | 4 | 89053047 | 99956611 | 0.1115 | 0.9723 | 2.9636 | 0.9603 | 3 | 1 |
| WCM161_Z1_1 | 4 | 99957208 | 100268901 | 0.726 | 1.6737 | NA | NA | NA | NA |
| WCM161_Z1_1 | 4 | 100334320 | 119273849 | −0.6937 | −0.0403 | 1.0148 | 0.9301 | 1 | 1 |
| WCM161_Z1_1 | 4 | 119472321 | 126428439 | 0.0938 | 0.9514 | 2.93 | 0.9374 | 3 | 1 |
| WCM161_Z1_1 | 4 | 128544639 | 139163217 | −0.6985 | −0.0468 | NA | NA | NA | NA |
| WCM161_Z1_1 | 4 | 139964292 | 146101105 | −0.2556 | 0.5283 | 1.7991 | 1.0853 | 2 | 1 |
| WCM161_Z1_1 | 4 | 146435939 | 164087801 | 0.0828 | 0.9384 | 2.8869 | 0.9458 | 3 | 1 |
| WCM161_Z1_1 | 4 | 164246608 | 166408605 | −0.6819 | −0.0242 | NA | NA | NA | NA |
| WCM161_Z1_1 | 4 | 166414352 | 178283623 | −0.0211 | 0.8146 | 2.5332 | 0.9844 | 3 | 1 |
| WCM161_Z1_1 | 4 | 178352874 | 187525108 | −0.6515 | 0.0169 | 1.0445 | 0.9791 | 1 | 1 |
| WCM161_Z1_1 | 4 | 187525689 | 190426502 | −1.2905 | −0.9325 | 0.9852 | 0.0627 | 1 | 0 |
| WCM161_Z1_1 | 5 | 140670 | 53751726 | 0.03 | 0.8756 | 1.9003 | 1.7692 | 2 | 2 |
| WCM161_Z1_1 | 5 | 53751988 | 121488130 | −0.2852 | 0.4914 | 1.8012 | 1.0105 | 2 | 1 |
| WCM161_Z1_1 | 5 | 121488405 | 180687804 | −0.08 | 0.7436 | 1.9215 | 1.4272 | 2 | 1 |
| WCM161_Z1_1 | 6 | 203545 | 170892833 | −0.2845 | 0.4923 | 1.8567 | 0.9566 | 2 | 1 |
| WCM161_Z1_1 | 7 | 193254 | 2515603 | −0.2812 | 0.4963 | 1.8828 | 0.9384 | 2 | 1 |
| WCM161_Z1_1 | 7 | 2516015 | 2582584 | −0.0533 | 0.7759 | NA | NA | NA | NA |
| WCM161_Z1_1 | 7 | 2582835 | 27134388 | 0.0183 | 0.8617 | 2.6543 | 0.9802 | 3 | 1 |
| WCM161_Z1_1 | 7 | 27134985 | 46729002 | 0.3344 | 1.2317 | 2.7426 | 1.9543 | 3 | 2 |
| WCM161_Z1_1 | 7 | 46732471 | 80123942 | −0.2844 | 0.4924 | 2.7658 | 0.0477 | 3 | 0 |
| WCM161_Z1_1 | 7 | 80141138 | 104377185 | 0.3103 | 1.2039 | 2.8267 | 1.7806 | 3 | 2 |
| WCM161_Z1_1 | 7 | 104485841 | 140259576 | −0.2901 | 0.4852 | 2.7903 | 0.0094 | 3 | 0 |
| WCM161_Z1_1 | 7 | 140267023 | 142498833 | −0.2893 | 0.4863 | 2.3242 | 0.4774 | 2 | 0 |
| WCM161_Z1_1 | 7 | 142499034 | 158937018 | −0.2516 | 0.5332 | 2.8896 | 0.0048 | 3 | 0 |
| WCM161_Z1_1 | 8 | 190907 | 26636925 | −0.7171 | −0.0722 | 1.8916 | 0.0108 | 2 | 0 |
| WCM161_Z1_1 | 8 | 26716715 | 26906371 | 1.3171 | 2.3173 | NA | NA | NA | NA |
| WCM161_Z1_1 | 8 | 26943771 | 32621431 | −0.7205 | −0.0769 | 1.872 | 0.0242 | 2 | 0 |
| WCM161_Z1_1 | 8 | 33230142 | 41542173 | 1.3848 | 2.3895 | 8.2089 | 2.271 | 8 | 2 |
| WCM161_Z1_1 | 8 | 41543690 | 146203933 | 1.5456 | 2.5606 | 10.0062 | 1.7927 | 10 | 2 |
| WCM161_Z1_1 | 8 | 146218375 | 146279489 | 1.3013 | 2.3003 | NA | NA | NA | NA |
| WCM161_Z1_1 | 9 | 17060 | 141111414 | 0.0553 | 0.9057 | 1.8868 | 1.8601 | 2 | 2 |
| WCM161_Z1_1 | X | 2707688 | 2833638 | 0.3187 | 1.2135 | NA | NA | NA | NA |
| WCM161_Z1_1 | X | 2836060 | 57936541 | 0.3209 | 1.2161 | NA | NA | NA | NA |
| WCM161_Z1_1 | X | 62898360 | 65835819 | 1.0279 | 2.0053 | NA | NA | NA | NA |
| WCM161_Z1_1 | X | 66765023 | 66943587 | 2.1478 | 3.1918 | NA | NA | NA | NA |
| WCM161_Z1_1 | X | 67268291 | 102005155 | −0.0093 | 0.8287 | NA | NA | NA | NA |
| WCM161_Z1_1 | X | 102005371 | 107979390 | 0.9681 | 1.9401 | NA | NA | NA | NA |
| WCM161_Z1_1 | X | 108619147 | 154509348 | −0.0426 | 0.7887 | NA | NA | NA | NA |
| WCM161_Z15_1 | 1 | 69569 | 49208397 | 0.0654 | 0.591 | 1.9954 | 1.0171 | 2 | 1 |
| WCM161_Z15_1 | 1 | 49224584 | 117753464 | −0.5234 | −0.0182 | 1.0081 | 0.9669 | 1 | 1 |
| WCM161_Z15_1 | 1 | 117911042 | 159824967 | 0.4214 | 0.9557 | 1.9593 | 1.9197 | 2 | 2 |
| WCM161_Z15_1 | 1 | 159825822 | 160458907 | −0.0578 | 0.4642 | NA | NA | NA | NA |
| WCM161_Z15_1 | 1 | 160460021 | 167334762 | 0.4295 | 0.9639 | 1.9506 | 1.9506 | 2 | 2 |
| WCM161_Z15_1 | 1 | 167339436 | 171765880 | −0.5161 | −0.0105 | 1.9847 | 7.00E−04 | 2 | 0 |
| WCM161_Z15_1 | 1 | 171810840 | 174992531 | −0.0129 | 0.5104 | 1.7939 | 1.055 | 2 | 1 |
| WCM161_Z15_1 | 1 | 175037064 | 178252762 | −0.5447 | −0.0404 | 1.9399 | 0.005 | 2 | 0 |
| WCM161_Z15_1 | 1 | 178269171 | 249212402 | 0.4167 | 0.9508 | 1.9329 | 1.9329 | 2 | 2 |
| WCM161_Z15_1 | 10 | 225981 | 5139649 | 0.0181 | 0.5423 | NA | NA | NA | NA |
| WCM161_Z15_1 | 10 | 5141056 | 9281413 | −0.5346 | −0.0298 | 1.959 | 2.00E−04 | 2 | 0 |
| WCM161_Z15_1 | 10 | 9449213 | 15902242 | 0.3018 | 0.8334 | 1.8001 | 1.7636 | 2 | 2 |
| WCM161_Z15_1 | 10 | 16526479 | 21414892 | −0.6326 | −0.1322 | 1.8217 | 0.0032 | 2 | 0 |
| WCM161_Z15_1 | 10 | 21415076 | 28527508 | 0.072 | 0.5977 | 1.8803 | 1.1463 | 2 | 1 |
| WCM161_Z15_1 | 10 | 28822933 | 29932918 | −0.5688 | −0.0655 | 1.931 | −0.0197 | 2 | 0 |
| WCM161_Z15_1 | 10 | 30306846 | 116197610 | 0.0222 | 0.5465 | 1.9352 | 0.9858 | 2 | 1 |
| WCM161_Z15_1 | 10 | 116198940 | 118187528 | −0.48 | 0.0271 | NA | NA | NA | NA |
| WCM161_Z15_1 | 10 | 118196258 | 135440068 | 0.0378 | 0.5625 | 1.9567 | 0.997 | 2 | 1 |
| WCM161_Z15_1 | 11 | 193100 | 93754655 | 0.0409 | 0.5657 | 1.961 | 0.9992 | 2 | 1 |
| WCM161_Z15_1 | 11 | 93778896 | 134856534 | −0.4994 | 0.0069 | 1.0184 | 0.9912 | 1 | 1 |
| WCM161_Z15_1 | 12 | 148674 | 11548434 | −0.5167 | −0.0111 | 1.0047 | 0.9799 | 1 | 1 |
| WCM161_Z15_1 | 12 | 11803072 | 20523087 | −1.4088 | −0.9561 | 1.0564 | −0.0255 | 1 | 0 |
| WCM161_Z15_1 | 12 | 20709602 | 21317791 | −0.0394 | 0.4832 | NA | NA | NA | NA |
| WCM161_Z15_1 | 12 | 21325705 | 30906565 | −1.4256 | −0.9742 | 0.9947 | 0.0233 | 1 | 0 |
| WCM161_Z15_1 | 12 | 30948848 | 100169404 | 0.0317 | 0.5563 | 1.9649 | 0.976 | 2 | 1 |
| WCM161_Z15_1 | 12 | 100173677 | 133810874 | −0.5042 | 0.0018 | 1.0127 | 0.9898 | 1 | 1 |
| WCM161_Z15_1 | 13 | 19600515 | 23238094 | 0.3953 | 0.929 | NA | NA | NA | NA |
| WCM161_Z15_1 | 13 | 23243302 | 37453532 | −0.4911 | 0.0155 | 2.0201 | 0.0015 | 2 | 0 |
| WCM161_Z15_1 | 13 | 37453784 | 41133922 | 0.3925 | 0.926 | NA | NA | NA | NA |
| WCM161_Z15_1 | 13 | 41134214 | 46563218 | −0.4278 | 0.0814 | 1.8938 | 0.2223 | 2 | 0 |
| WCM161_Z15_1 | 13 | 46577368 | 58299420 | −1.417 | −0.9649 | 1.0198 | 0.0049 | 1 | 0 |
| WCM161_Z15_1 | 13 | 60240961 | 67802561 | −0.6261 | −0.1253 | NA | NA | NA | NA |
| WCM161_Z15_1 | 13 | 70281780 | 95271437 | −0.012 | 0.5113 | 1.9168 | 0.9339 | 2 | 1 |
| WCM161_Z15_1 | 13 | 95271757 | 101316546 | −1.4635 | −1.0153 | 1.0049 | −0.0154 | 1 | 0 |
| WCM161_Z15_1 | 13 | 101318340 | 115089411 | 0.0536 | 0.5788 | 1.9742 | 1.0131 | 2 | 1 |
| WCM161_Z15_1 | 14 | 19377697 | 51721141 | 0.438 | 0.9725 | 1.9623 | 1.9623 | 2 | 2 |
| WCM161_Z15_1 | 14 | 52156636 | 53619550 | −1.2608 | −0.7967 | NA | NA | NA | NA |
| WCM161_Z15_1 | 14 | 53619818 | 63920440 | −0.0363 | 0.4863 | 1.7975 | 1.0041 | 2 | 1 |
| WCM161_Z15_1 | 14 | 64006292 | 64749659 | −1.5901 | −1.1531 | NA | NA | NA | NA |
| WCM161_Z15_1 | 14 | 64805001 | 107283026 | 0.0451 | 0.5701 | 1.9578 | 1.0115 | 2 | 1 |
| WCM161_Z15_1 | 15 | 20170036 | 102506273 | 0.0355 | 0.5601 | 2.9602 | −0.0113 | 3 | 0 |
| WCM161_Z15_1 | 16 | 67604 | 72198810 | 0.0452 | 0.5702 | 1.9778 | 0.9917 | 2 | 1 |
| WCM161_Z15_1 | 16 | 72821271 | 90233622 | −1.41 | −0.9573 | 1.0322 | −0.0021 | 1 | 0 |
| WCM161_Z15_1 | 17 | 131595 | 21194820 | −0.5213 | −0.0159 | 1.9888 | −0.0107 | 2 | 0 |
| WCM161_Z15_1 | 17 | 21199022 | 77093792 | 0.4471 | 0.9819 | 1.9892 | 1.961 | 2 | 2 |
| WCM161_Z15_1 | 17 | 77097678 | 81083519 | 0.0558 | 0.5811 | 2.0295 | 0.9624 | 2 | 1 |
| WCM161_Z15_1 | 18 | 48338 | 2847830 | 1.0255 | 1.5703 | NA | NA | NA | NA |
| WCM161_Z15_1 | 18 | 2885118 | 78005219 | 0.4141 | 0.9481 | 1.9294 | 1.9294 | 2 | 2 |
| WCM161_Z15_1 | 19 | 281499 | 5456450 | −0.5081 | −0.0022 | 1.0963 | 0.9007 | 1 | 1 |
| WCM161_Z15_1 | 19 | 5456683 | 6392236 | 0.092 | 0.6182 | 2.2019 | 0.8681 | 2 | 1 |
| WCM161_Z15_1 | 19 | 6392905 | 59083923 | −0.5046 | 0.0014 | 1.0771 | 0.9249 | 1 | 1 |
| WCM161_Z15_1 | 2 | 41612 | 205969220 | 0.0261 | 0.5505 | 1.9572 | 0.9721 | 2 | 1 |
| WCM161_Z15_1 | 2 | 205978257 | 242946607 | −0.5199 | −0.0145 | 1.0034 | 0.9766 | 1 | 1 |
| WCM161_Z15_1 | 20 | 68396 | 59091758 | 0.442 | 0.9766 | 1.9679 | 1.9679 | 2 | 2 |
| WCM161_Z15_1 | 20 | 59139674 | 62904890 | 0.7431 | 1.2835 | 2.8305 | 2.0382 | 3 | 2 |
| WCM161_Z15_1 | 21 | 9826235 | 48084226 | −0.4612 | 0.0466 | 2.0644 | 0.0013 | 2 | 0 |
| WCM161_Z15_1 | 22 | 17063440 | 18912678 | −0.0909 | 0.4301 | 1.8501 | 0.8445 | 2 | 1 |
| WCM161_Z15_1 | 22 | 18913224 | 29537989 | −0.4695 | 0.038 | 2.0524 | 0.0011 | 2 | 0 |
| WCM161_Z15_1 | 22 | 29563074 | 30188461 | −1.3187 | −0.8589 | NA | NA | NA | NA |
| WCM161_Z15_1 | 22 | 30189467 | 51215157 | −0.4896 | 0.017 | 2.0233 | 5.00E−04 | 2 | 0 |
| WCM161_Z15_1 | 3 | 239431 | 88205442 | −0.5048 | 0.0012 | 1.0113 | 0.9904 | 1 | 1 |
| WCM161_Z15_1 | 3 | 88205677 | 115805400 | 0.0321 | 0.5567 | 1.9338 | 1.0081 | 2 | 1 |
| WCM161_Z15_1 | 3 | 116163765 | 133098806 | −0.5164 | −0.0108 | 1.0039 | 0.9811 | 1 | 1 |
| WCM161_Z15_1 | 3 | 133099060 | 197712716 | 0.0133 | 0.5373 | 1.9365 | 0.9661 | 2 | 1 |
| WCM161_Z15_1 | 3 | 197717503 | 197894719 | −0.2125 | 0.3045 | NA | NA | NA | NA |
| WCM161_Z15_1 | 4 | 59331 | 46067515 | 0.053 | 0.5782 | 1.9871 | 0.9988 | 2 | 1 |
| WCM161_Z15_1 | 4 | 46086060 | 46737146 | −0.2663 | 0.2489 | NA | NA | NA | NA |
| WCM161_Z15_1 | 4 | 46930289 | 71835721 | 0.7355 | 1.2758 | 2.9065 | 1.9361 | 3 | 2 |
| WCM161_Z15_1 | 4 | 71840896 | 84216579 | 0.3931 | 0.9268 | 1.901 | 1.901 | 2 | 2 |
| WCM161_Z15_1 | 4 | 84222243 | 87968198 | −0.1706 | 0.3478 | NA | NA | NA | NA |
| WCM161_Z15_1 | 4 | 87968475 | 122743637 | 0.3716 | 0.9047 | 1.8913 | 1.8531 | 2 | 2 |
| WCM161_Z15_1 | 4 | 122744682 | 124323610 | 0.0328 | 0.5574 | NA | NA | NA | NA |
| WCM161_Z15_1 | 4 | 125590293 | 169606664 | 0.3907 | 0.9243 | 1.8977 | 1.8977 | 2 | 2 |
| WCM161_Z15_1 | 4 | 169611875 | 187525598 | 0.0033 | 0.5271 | 1.8784 | 1.0036 | 2 | 1 |
| WCM161_Z15_1 | 4 | 187527266 | 190426502 | −1.3441 | −0.8862 | 1.0783 | 0.0038 | 1 | 0 |
| WCM161_Z15_1 | 5 | 140515 | 180687769 | 0.0203 | 0.5446 | 1.9342 | 0.983 | 2 | 1 |
| WCM161_Z15_1 | 6 | 203510 | 66205263 | 0.0463 | 0.5713 | 1.9609 | 1.0108 | 2 | 1 |
| WCM161_Z15_1 | 6 | 68599049 | 170190670 | −0.5281 | −0.023 | 0.9965 | 0.9719 | 1 | 1 |
| WCM161_Z15_1 | 6 | 170592124 | 170892615 | −0.0175 | 0.5057 | NA | NA | NA | NA |
| WCM161_Z15_1 | 7 | 193253 | 48407419 | 0.4133 | 0.9473 | 1.9283 | 1.9283 | 2 | 2 |
| WCM161_Z15_1 | 7 | 48411945 | 48556375 | 0.01 | 0.5339 | NA | NA | NA | NA |
| WCM161_Z15_1 | 7 | 48559666 | 77256361 | −0.5569 | −0.053 | 1.8488 | 0.079 | 2 | 0 |
| WCM161_Z15_1 | 7 | 77256629 | 94146940 | 0.1098 | 0.6365 | 2.1691 | 0.94 | 2 | 1 |
| WCM161_Z15_1 | 7 | 94147633 | 104377207 | 0.4949 | 1.0307 | 2.2369 | 1.8491 | 2 | 2 |
| WCM161_Z15_1 | 7 | 104485841 | 141490841 | −0.5145 | −0.0088 | 1.9881 | −2.00E−04 | 2 | 0 |
| WCM161_Z15_1 | 7 | 141490992 | 143632618 | −0.4961 | 0.0103 | 1.7298 | 0.2845 | 2 | 0 |
| WCM161_Z15_1 | 7 | 143632984 | 158937227 | −0.5257 | −0.0206 | 1.9815 | −0.0098 | 2 | 0 |
| WCM161_Z15_1 | 8 | 190897 | 35584004 | −1.4214 | −0.9696 | 1.0232 | −0.0019 | 1 | 0 |
| WCM161_Z15_1 | 8 | 35588556 | 40011108 | 1.2319 | 1.7794 | NA | NA | NA | NA |
| WCM161_Z15_1 | 8 | 40011312 | 82751902 | 0.6972 | 1.2368 | 3.7599 | 0.9537 | 4 | 1 |
| WCM161_Z15_1 | 8 | 82752176 | 86556651 | 1.1819 | 1.7288 | NA | NA | NA | NA |
| WCM161_Z15_1 | 8 | 86575713 | 125498427 | 1.1882 | 1.7352 | 5.7158 | 0.9426 | 6 | 1 |
| WCM161_Z15_1 | 8 | 125498761 | 146279472 | 1.416 | 1.9656 | 5.8716 | 1.9399 | 6 | 2 |
| WCM161_Z15_1 | 9 | 17084 | 110081149 | 0.4487 | 0.9835 | 2.0014 | 1.953 | 2 | 2 |
| WCM161_Z15_1 | 9 | 110084261 | 119488084 | 0.0405 | 0.5653 | 2.0244 | 0.9351 | 2 | 1 |
| WCM161_Z15_1 | 9 | 119491319 | 126774736 | 0.4126 | 0.9466 | 1.949 | 1.9057 | 2 | 2 |
| WCM161_Z15_1 | 9 | 126776323 | 127661645 | 0.9721 | 1.5161 | NA | NA | NA | NA |
| WCM161_Z15_1 | 9 | 127662748 | 141111414 | 0.4811 | 1.0166 | 2.0231 | 2.0231 | 2 | 2 |
| WCM161_Z15_1 | X | 2827931 | 9936131 | 1.6889 | 2.2411 | NA | NA | NA | NA |
| WCM161_Z15_1 | X | 10035339 | 154509348 | 0.3033 | 0.8349 | NA | NA | NA | NA |
| WCM161_Z6_1 | 1 | 69590 | 39893161 | −0.1329 | 0.9411 | 2.7681 | 1.0718 | 3 | 1 |
| WCM161_Z6_1 | 1 | 39893768 | 111738552 | −0.3665 | 0.4945 | 2.8457 | −0.0281 | 3 | 0 |
| WCM161_Z6_1 | 1 | 111739863 | 111862042 | −0.052 | 1.0822 | NA | NA | NA | NA |
| WCM161_Z6_1 | 1 | 111862872 | 120510847 | −0.1278 | 0.9501 | 2.6871 | 1.177 | 3 | 1 |
| WCM161_Z6_1 | 1 | 120611425 | 167356420 | 0.1006 | 1.3338 | 2.8584 | 2.183 | 3 | 2 |
| WCM161_Z6_1 | 1 | 167358837 | 178063703 | −0.302 | 0.6245 | 2.8679 | 0.2155 | 3 | 0 |
| WCM161_Z6_1 | 1 | 178252762 | 249212388 | 0.0766 | 1.2955 | 2.5021 | 2.4072 | 3 | 2 |
| WCM161_Z6_1 | 10 | 225952 | 9281413 | −0.4614 | 0.2911 | 2.3048 | 0.1423 | 2 | 0 |
| WCM161_Z6_1 | 10 | 9449213 | 15902234 | −0.1057 | 0.9892 | 2.0757 | 1.8945 | 2 | 2 |
| WCM161_Z6_1 | 10 | 16526479 | 38122001 | −0.3842 | 0.4576 | 2.057 | 0.6894 | 2 | 1 |
| WCM161_Z6_1 | 10 | 38126632 | 75673879 | −0.036 | 1.1094 | 2.1978 | 2.1173 | 2 | 2 |
| WCM161_Z6_1 | 10 | 75674588 | 75863605 | −0.361 | 0.5058 | NA | NA | NA | NA |
| WCM161_Z6_1 | 10 | 75864906 | 76781722 | 0.0556 | 1.2614 | NA | NA | NA | NA |
| WCM161_Z6_1 | 10 | 76784820 | 96698603 | 0.3335 | 1.6895 | 3.7499 | 2.7009 | 4 | 3 |
| WCM161_Z6_1 | 10 | 96701711 | 135043989 | 0.0857 | 1.31 | 3.8364 | 1.1224 | 4 | 1 |
| WCM161_Z6_1 | 10 | 135044881 | 135440068 | −0.0707 | 1.0501 | NA | NA | NA | NA |
| WCM161_Z6_1 | 11 | 193135 | 6341365 | −0.0473 | 1.0902 | 4.1505 | 0.1077 | 4 | 0 |
| WCM161_Z6_1 | 11 | 6341675 | 7111497 | 0.1763 | 1.4528 | 4.1526 | 1.322 | 4 | 1 |
| WCM161_Z6_1 | 11 | 7256013 | 28143692 | −0.054 | 1.0788 | 4.2111 | 0.0136 | 4 | 0 |
| WCM161_Z6_1 | 11 | 28219089 | 99786841 | 0.1612 | 1.4295 | 4.2199 | 1.167 | 4 | 1 |
| WCM161_Z6_1 | 11 | 99827584 | 113320799 | −0.2014 | 0.8168 | 2.6192 | 0.9037 | 3 | 1 |
| WCM161_Z6_1 | 11 | 113558990 | 134856603 | 0.1959 | 1.4831 | 4.4706 | 1.1202 | 4 | 1 |
| WCM161_Z6_1 | 12 | 148644 | 12232263 | −0.2587 | 0.7087 | 2.1986 | 1.0702 | 2 | 1 |
| WCM161_Z6_1 | 12 | 12232563 | 31079921 | −0.5617 | 0.0575 | 2.0289 | 0.0523 | 2 | 0 |
| WCM161_Z6_1 | 12 | 31107026 | 133779852 | −0.0502 | 1.0852 | 2.1579 | 2.0855 | 2 | 2 |
| WCM161_Z6_1 | 12 | 133780161 | 133810868 | −0.2809 | 0.6659 | NA | NA | NA | NA |
| WCM161_Z6_1 | 13 | 19600515 | 22178275 | 1.3703 | 3.0347 | NA | NA | NA | NA |
| WCM161_Z6_1 | 13 | 22246093 | 26145827 | −0.8334 | −0.7261 | 1.1517 | 0.0573 | 1 | 0 |
| WCM161_Z6_1 | 13 | 26148963 | 28368049 | −0.0377 | 1.1065 | NA | NA | NA | NA |
| WCM161_Z6_1 | 13 | 28494281 | 34098379 | −0.7851 | −0.5644 | 1.6751 | −0.3227 | 2 | 0 |
| WCM161_Z6_1 | 13 | 34392395 | 35517179 | 0.5218 | 1.9576 | NA | NA | NA | NA |
| WCM161_Z6_1 | 13 | 35615073 | 37453779 | −0.9112 | −1.0175 | NA | NA | NA | NA |
| WCM161_Z6_1 | 13 | 37524190 | 40362473 | 1.1656 | 2.7891 | NA | NA | NA | NA |
| WCM161_Z6_1 | 13 | 41133851 | 42893343 | −0.8707 | −0.8605 | NA | NA | NA | NA |
| WCM161_Z6_1 | 13 | 43148455 | 46616337 | 1.2857 | 2.9341 | NA | NA | NA | NA |
| WCM161_Z6_1 | 13 | 46619175 | 55748666 | −0.8669 | −0.8463 | 1.0559 | 0.0565 | 1 | 0 |
| WCM161_Z6_1 | 13 | 58206799 | 95235475 | −0.41 | 0.4031 | 1.4183 | 1.2264 | 1 | 1 |
| WCM161_Z6_1 | 13 | 95244526 | 101287127 | −0.922 | −1.0614 | 1.1125 | −0.1542 | 1 | 0 |
| WCM161_Z6_1 | 13 | 101287404 | 115089411 | −0.1501 | 0.9103 | 2.6432 | 1.1157 | 3 | 1 |
| WCM161_Z6_1 | 14 | 19377712 | 50998731 | 0.1208 | 1.3659 | 3.1034 | 2.0514 | 3 | 2 |
| WCM161_Z6_1 | 14 | 51027017 | 87387822 | −0.2978 | 0.6329 | 2.1456 | 0.9558 | 2 | 1 |
| WCM161_Z6_1 | 14 | 88400339 | 107283019 | 0.177 | 1.4539 | 3.3925 | 2.0867 | 3 | 2 |
| WCM161_Z6_1 | 15 | 20170134 | 102506284 | −0.2925 | 0.6433 | 2.1375 | 0.9863 | 2 | 1 |
| WCM161_Z6_1 | 16 | 67604 | 72827432 | −0.0624 | 1.0644 | 2.1559 | 2.0266 | 2 | 2 |
| WCM161_Z6_1 | 16 | 72827758 | 90233622 | −0.5499 | 0.0861 | 2.0886 | 0.0343 | 2 | 0 |
| WCM161_Z6_1 | 17 | 131595 | 21194820 | −0.2947 | 0.6389 | 3.1774 | −0.0632 | 3 | 0 |
| WCM161_Z6_1 | 17 | 21199037 | 21319868 | 0.0628 | 1.2731 | 2.6479 | 2.1857 | 3 | 2 |
| WCM161_Z6_1 | 17 | 21438747 | 71746796 | 0.1375 | 1.3922 | 3.1611 | 2.0886 | 3 | 2 |
| WCM161_Z6_1 | 17 | 71757115 | 74921130 | 0.3059 | 1.6488 | 3.2423 | 3.029 | 3 | 3 |
| WCM161_Z6_1 | 17 | 74922755 | 81083381 | −0.0494 | 1.0866 | 3.2979 | 0.9497 | 3 | 1 |
| WCM161_Z6_1 | 18 | 48338 | 216486 | 0.0665 | 1.2792 | NA | NA | NA | NA |
| WCM161_Z6_1 | 18 | 218887 | 15006027 | 0.4897 | 1.9129 | 5.7245 | 1.8068 | 6 | 2 |
| WCM161_Z6_1 | 18 | 18531345 | 31324171 | 0.1846 | 1.4657 | 3.6272 | 1.8968 | 4 | 2 |
| WCM161_Z6_1 | 18 | 31324563 | 39647378 | 0.4068 | 1.7956 | 6.1065 | 0.8366 | 6 | 1 |
| WCM161_Z6_1 | 18 | 40323456 | 46343666 | 0.0516 | 1.255 | 3.5099 | 1.2634 | 4 | 1 |
| WCM161_Z6_1 | 18 | 46383964 | 78005224 | −0.4228 | 0.3755 | 1.384 | 1.2106 | 1 | 1 |
| WCM161_Z6_1 | 19 | 281445 | 651785 | −0.5568 | 0.0694 | 2.1787 | −0.08 | 2 | 0 |
| WCM161_Z6_1 | 19 | 652238 | 1581250 | 0.4225 | 1.8181 | 4.6117 | 2.4404 | 5 | 2 |
| WCM161_Z6_1 | 19 | 1582673 | 9251258 | −0.5375 | 0.1157 | 2.1014 | 0.0656 | 2 | 0 |
| WCM161_Z6_1 | 19 | 9266691 | 9406900 | 0.5188 | 1.9534 | NA | NA | NA | NA |
| WCM161_Z6_1 | 19 | 9406953 | 14991963 | 0.4787 | 1.8975 | 5.3091 | 2.1423 | 5 | 2 |
| WCM161_Z6_1 | 19 | 14992157 | 34304214 | 0.182 | 1.4617 | 3.303 | 2.2054 | 3 | 2 |
| WCM161_Z6_1 | 19 | 34513673 | 57088843 | −0.5341 | 0.124 | 2.1878 | −0.0083 | 2 | 0 |
| WCM161_Z6_1 | 19 | 57088850 | 59083985 | −0.268 | 0.6909 | 2.2122 | 1.0163 | 2 | 1 |
| WCM161_Z6_1 | 2 | 41612 | 242946607 | −0.281 | 0.6658 | 2.1482 | 1.0247 | 2 | 1 |
| WCM161_Z6_1 | 20 | 68406 | 62904835 | −0.054 | 1.0788 | 2.1268 | 2.0979 | 2 | 2 |
| WCM161_Z6_1 | 21 | 9826235 | 39822803 | −0.2284 | 0.7663 | 2.45 | 0.9519 | 2 | 1 |
| WCM161_Z6_1 | 21 | 39947594 | 42866388 | 0.7651 | 2.2848 | 8.7284 | 1.0173 | 9 | 1 |
| WCM161_Z6_1 | 21 | 42870082 | 48084277 | −0.2704 | 0.6862 | 2.1873 | 1.0307 | 2 | 1 |
| WCM161_Z6_1 | 22 | 17063485 | 18256374 | −0.2692 | 0.6886 | NA | NA | NA | NA |
| WCM161_Z6_1 | 22 | 18257224 | 40831521 | 0.1049 | 1.3407 | 2.807 | 2.2585 | 3 | 2 |
| WCM161_Z6_1 | 22 | 40859256 | 51215134 | −0.2901 | 0.6479 | 2.2048 | 0.929 | 2 | 1 |
| WCM161_Z6_1 | 3 | 239377 | 7603260 | −0.1328 | 0.9412 | NA | NA | NA | NA |
| WCM161_Z6_1 | 3 | 7620382 | 8705555 | 0.2888 | 1.6235 | NA | NA | NA | NA |
| WCM161_Z6_1 | 3 | 8757555 | 49679822 | 0.1289 | 1.3788 | 4.2653 | 0.9357 | 4 | 1 |
| WCM161_Z6_1 | 3 | 49680066 | 87289860 | 0.2911 | 1.6269 | 4.1575 | 2.0193 | 4 | 2 |
| WCM161_Z6_1 | 3 | 87295049 | 170585946 | 0.073 | 1.2897 | 2.7958 | 2.0936 | 3 | 2 |
| WCM161_Z6_1 | 3 | 170586128 | 197894726 | −0.0956 | 1.0069 | 2.0904 | 1.9289 | 2 | 2 |
| WCM161_Z6_1 | 4 | 59331 | 8098834 | −0.0621 | 1.0649 | 3.1409 | 1.0432 | 3 | 1 |
| WCM161_Z6_1 | 4 | 8108325 | 49046830 | −0.2882 | 0.6517 | 2.1453 | 0.9967 | 2 | 1 |
| WCM161_Z6_1 | 4 | 49052752 | 55127300 | 0.4755 | 1.893 | NA | NA | NA | NA |
| WCM161_Z6_1 | 4 | 55127533 | 57976281 | −0.3407 | 0.5472 | NA | NA | NA | NA |
| WCM161_Z6_1 | 4 | 57976427 | 77305759 | 0.7413 | 2.2536 | 8.5676 | 0.9699 | 9 | 1 |
| WCM161_Z6_1 | 4 | 77324327 | 82090890 | −0.3176 | 0.5936 | 1.6685 | 1.3495 | 2 | 1 |
| WCM161_Z6_1 | 4 | 82092915 | 89052995 | 0.6709 | 2.1603 | 8.2016 | 0.7388 | 8 | 1 |
| WCM161_Z6_1 | 4 | 89053781 | 119472275 | −0.2832 | 0.6613 | 2.1054 | 1.0577 | 2 | 1 |
| WCM161_Z6_1 | 4 | 119606698 | 126428439 | 0.6241 | 2.0976 | 7.4992 | 1.0605 | 7 | 1 |
| WCM161_Z6_1 | 4 | 128544578 | 139966271 | −0.2481 | 0.729 | NA | NA | NA | NA |
| WCM161_Z6_1 | 4 | 139966467 | 164087809 | 0.5101 | 1.9413 | 6.3931 | 1.2877 | 6 | 1 |
| WCM161_Z6_1 | 4 | 164246708 | 166408681 | −0.3494 | 0.5296 | NA | NA | NA | NA |
| WCM161_Z6_1 | 4 | 166414332 | 178911657 | 0.5103 | 1.9416 | 6.7684 | 0.9143 | 7 | 1 |
| WCM161_Z6_1 | 4 | 183090455 | 190426502 | −0.3304 | 0.5679 | 2.1326 | 0.8322 | 2 | 1 |
| WCM161_Z6_1 | 5 | 140627 | 228448 | −0.0321 | 1.116 | NA | NA | NA | NA |
| WCM161_Z6_1 | 5 | 231001 | 142254679 | 0.0756 | 1.2938 | 2.5031 | 2.4003 | 3 | 2 |
| WCM161_Z6_1 | 5 | 142258986 | 180687814 | −0.1137 | 0.9752 | 1.9971 | 1.9347 | 2 | 2 |
| WCM161_Z6_1 | 6 | 203466 | 170892825 | −0.2916 | 0.645 | 2.0601 | 1.0674 | 2 | 1 |
| WCM161_Z6_1 | 7 | 193265 | 12417407 | 0.0057 | 1.1794 | 2.7542 | 1.7754 | 3 | 2 |
| WCM161_Z6_1 | 7 | 12419108 | 45148667 | 0.3257 | 1.678 | 4.4078 | 1.992 | 4 | 2 |
| WCM161_Z6_1 | 7 | 45148773 | 45960731 | 0.2856 | 1.6188 | NA | NA | NA | NA |
| WCM161_Z6_1 | 7 | 45961271 | 82582228 | −0.0315 | 1.117 | 4.1025 | 0.2354 | 4 | 0 |
| WCM161_Z6_1 | 7 | 82582513 | 104681442 | 0.2426 | 1.5542 | 3.7885 | 2.0848 | 4 | 2 |
| WCM161_Z6_1 | 7 | 104702680 | 138593771 | −0.1612 | 0.8902 | 3.8286 | −0.1217 | 4 | 0 |
| WCM161_Z6_1 | 7 | 138596064 | 143632984 | −0.0859 | 1.0237 | 3.7538 | 0.3125 | 4 | 0 |
| WCM161_Z6_1 | 7 | 143633157 | 158937011 | −0.1312 | 0.9441 | 3.862 | −0.014 | 4 | 0 |
| WCM161_Z6_1 | 8 | 190897 | 32621438 | −0.5747 | 0.0255 | 2.1181 | −0.0825 | 2 | 0 |
| WCM161_Z6_1 | 8 | 33230313 | 35453147 | 1.3199 | 2.9749 | NA | NA | NA | NA |
| WCM161_Z6_1 | 8 | 35541126 | 126370010 | 1.2418 | 2.8814 | 12.5478 | 2.1893 | 13 | 2 |
| WCM161_Z6_1 | 8 | 126379041 | 146279479 | 1.3846 | 3.0516 | 14.284 | 2.2991 | 14 | 2 |
| WCM161_Z6_1 | 9 | 17101 | 141111414 | 0.0219 | 1.2062 | 2.3377 | 2.2768 | 2 | 2 |
| WCM161_Z6_1 | X | 2707682 | 14937881 | 0.0083 | 1.1837 | NA | NA | NA | NA |
| WCM161_Z6_1 | X | 15262636 | 37641416 | 0.9644 | 2.5403 | NA | NA | NA | NA |
| WCM161_Z6_1 | X | 37642778 | 42637017 | 1.468 | 3.1495 | NA | NA | NA | NA |
| WCM161_Z6_1 | X | 42637040 | 64137685 | 0.9294 | 2.4961 | NA | NA | NA | NA |
| WCM161_Z6_1 | X | 64138962 | 67652739 | 2.3359 | 4.1237 | NA | NA | NA | NA |
| WCM161_Z6_1 | X | 67731711 | 154509348 | −0.1036 | 0.9928 | NA | NA | NA | NA |
| WCM162 | 1 | 14930 | 1002932 | −0.2114 | −0.0502 | 1.1258 | 0.8058 | 1 | 1 |
| WCM162 | 1 | 1004565 | 1636379 | −0.6388 | −0.9304 | 1.0159 | 0.0335 | 1 | 0 |
| WCM162 | 1 | 1638780 | 3683090 | 0.2638 | 0.7109 | 1.7229 | 1.5507 | 2 | 2 |
| WCM162 | 1 | 3683747 | 11354924 | −0.2108 | −0.0492 | 0.9665 | 0.9665 | 1 | 1 |
| WCM162 | 1 | 11561038 | 13933849 | 0.2561 | 0.6996 | 1.7359 | 1.5123 | 2 | 2 |
| WCM162 | 1 | 13936822 | 17313157 | −0.1231 | 0.1035 | 1.1128 | 1.036 | 1 | 1 |
| WCM162 | 1 | 17313454 | 20680271 | 0.2308 | 0.6625 | 1.688 | 1.4777 | 2 | 1 |
| WCM162 | 1 | 20680662 | 21175861 | −0.2773 | −0.1698 | 0.9481 | 0.8298 | 1 | 1 |
| WCM162 | 1 | 21177773 | 24022911 | −0.6247 | −0.8962 | 1.0036 | 0.071 | 1 | 0 |
| WCM162 | 1 | 24075460 | 29010295 | −0.16 | 0.0401 | 1.0282 | 1.0282 | 1 | 1 |
| WCM162 | 1 | 29016716 | 31186387 | 0.542 | 1.1013 | NA | NA | NA | NA |
| WCM162 | 1 | 31187116 | 55969890 | −0.1922 | −0.0162 | 0.9889 | 0.9889 | 1 | 1 |
| WCM162 | 1 | 55970134 | 91182847 | 0.0613 | 0.4046 | 1.3237 | 1.3237 | 1 | 1 |
| WCM162 | 1 | 91380919 | 92498024 | 0.6584 | 1.2565 | 3.8384 | 0.94 | 4 | 1 |
| WCM162 | 1 | 92508428 | 99418879 | −0.2823 | −0.179 | 0.8833 | 0.8833 | 1 | 1 |
| WCM162 | 1 | 99422203 | 110876376 | 0.0548 | 0.3943 | 1.4033 | 1.2254 | 1 | 1 |
| WCM162 | 1 | 110882174 | 145687061 | −0.1198 | 0.1091 | 1.0785 | 1.0785 | 1 | 1 |
| WCM162 | 1 | 145687155 | 152384759 | 0.3428 | 0.8249 | 1.822 | 1.721 | 2 | 2 |
| WCM162 | 1 | 152483959 | 157963503 | −0.0395 | 0.243 | 1.1835 | 1.1835 | 1 | 1 |
| WCM162 | 1 | 158045072 | 160209979 | 0.6745 | 1.2777 | 3.5848 | 1.2642 | 4 | 1 |
| WCM162 | 1 | 160210223 | 162345189 | 0.3502 | 0.8354 | 2.3455 | 1.2233 | 2 | 1 |
| WCM162 | 1 | 162346496 | 163046227 | 0.1705 | 0.5725 | NA | NA | NA | NA |
| WCM162 | 1 | 163114605 | 169890854 | −0.3388 | −0.2856 | 1.5857 | 0.055 | 2 | 0 |
| WCM162 | 1 | 169923219 | 181724537 | −0.6337 | −0.9179 | 1.0427 | 0.0158 | 1 | 0 |
| WCM162 | 1 | 181725167 | 183853056 | −0.2011 | −0.0319 | 1.9847 | −0.0285 | 2 | 0 |
| WCM162 | 1 | 183853985 | 203819595 | 0.1076 | 0.4767 | 1.4388 | 1.3444 | 1 | 1 |
| WCM162 | 1 | 203820303 | 207532965 | 0.6028 | 1.1829 | 3.4136 | 1.127 | 3 | 1 |
| WCM162 | 1 | 207635031 | 222892214 | −0.0253 | 0.2662 | 1.2026 | 1.2026 | 1 | 1 |
| WCM162 | 1 | 222892601 | 228612921 | 0.2608 | 0.7066 | 1.8611 | 1.4029 | 2 | 1 |
| WCM162 | 1 | 228645113 | 237994957 | −0.1902 | −0.0126 | 0.9913 | 0.9913 | 1 | 1 |
| WCM162 | 1 | 237995938 | 246021881 | 0.2643 | 0.7117 | 1.731 | 1.5444 | 2 | 2 |
| WCM162 | 1 | 246027253 | 249213967 | −0.1159 | 0.1158 | 1.0836 | 1.0836 | 1 | 1 |
| WCM162 | 10 | 225910 | 43089731 | −0.073 | 0.1877 | 1.139 | 1.139 | 1 | 1 |
| WCM162 | 10 | 43089949 | 54074415 | −0.59 | −0.8141 | 1.1496 | −0.0121 | 1 | 0 |
| WCM162 | 10 | 54074660 | 69647342 | −0.132 | 0.0883 | 1.0631 | 1.0631 | 1 | 1 |
| WCM162 | 10 | 69648749 | 71835399 | 0.2871 | 0.7448 | 1.7657 | 1.5859 | 2 | 2 |
| WCM162 | 10 | 71849793 | 79814784 | −0.0649 | 0.2011 | 1.1496 | 1.1496 | 1 | 1 |
| WCM162 | 10 | 80029759 | 82405911 | 0.4591 | 0.9881 | 2.7256 | 1.2416 | 3 | 1 |
| WCM162 | 10 | 83635560 | 97810219 | −0.0928 | 0.1547 | 1.1132 | 1.1132 | 1 | 1 |
| WCM162 | 10 | 97816773 | 102283647 | 0.504 | 1.0497 | 2.8468 | 1.2934 | 3 | 1 |
| WCM162 | 10 | 102286222 | 102510673 | 0.1344 | 0.5178 | NA | NA | NA | NA |
| WCM162 | 10 | 102539321 | 105048293 | −0.2956 | −0.2038 | 0.8683 | 0.8683 | 1 | 1 |
| WCM162 | 10 | 105048435 | 108334796 | 0.1981 | 0.6139 | 1.816 | 1.2448 | 2 | 1 |
| WCM162 | 10 | 108335627 | 120907203 | −0.6421 | −0.9384 | 0.9455 | 0.0981 | 1 | 0 |
| WCM162 | 10 | 120914530 | 123239438 | −0.1276 | 0.0958 | 1.22 | 0.9174 | 1 | 1 |
| WCM162 | 10 | 123241737 | 134981837 | 0.2653 | 0.7131 | 1.7422 | 1.5364 | 2 | 2 |
| WCM162 | 10 | 134996974 | 135477898 | −0.1939 | −0.0193 | 1.0703 | 0.9032 | 1 | 1 |
| WCM162 | 11 | 193096 | 1021268 | −0.1529 | 0.0525 | 1.0908 | 0.9833 | 1 | 1 |
| WCM162 | 11 | 1023485 | 2905091 | 0.2911 | 0.7507 | 2.1626 | 1.2025 | 2 | 1 |
| WCM162 | 11 | 2905297 | 9519351 | −0.1874 | −0.0077 | 0.9947 | 0.9947 | 1 | 1 |
| WCM162 | 11 | 9522712 | 18108492 | 0.1195 | 0.4949 | 1.4633 | 1.3551 | 1 | 1 |
| WCM162 | 11 | 18108687 | 43340140 | −0.1466 | 0.0632 | 1.0448 | 1.0448 | 1 | 1 |
| WCM162 | 11 | 43342439 | 46473454 | 0.1928 | 0.606 | 1.736 | 1.308 | 2 | 1 |
| WCM162 | 11 | 46515196 | 47799857 | −0.3097 | −0.2302 | 0.8573 | 0.8477 | 1 | 1 |
| WCM162 | 11 | 47800351 | 55036774 | 0.3009 | 0.7648 | 2.0671 | 1.3311 | 2 | 1 |
| WCM162 | 11 | 55038033 | 62296346 | −0.1043 | 0.1353 | 1.0983 | 1.0983 | 1 | 1 |
| WCM162 | 11 | 62296703 | 65743928 | −0.3245 | −0.2582 | 0.8362 | 0.8362 | 1 | 1 |
| WCM162 | 11 | 65744493 | 67012860 | 0.053 | 0.3916 | 1.4854 | 1.1383 | 1 | 1 |
| WCM162 | 11 | 67013600 | 70013506 | 0.6188 | 1.2041 | 3.1236 | 1.4843 | 3 | 1 |
| WCM162 | 11 | 70014114 | 113566153 | −0.1433 | 0.0689 | 1.0489 | 1.0489 | 1 | 1 |
| WCM162 | 11 | 113567679 | 117076026 | −0.4464 | −0.4997 | 0.7935 | 0.621 | 1 | 1 |
| WCM162 | 11 | 117076048 | 134605954 | −0.0434 | 0.2366 | 1.1782 | 1.1782 | 1 | 1 |
| WCM162 | 12 | 148452 | 2602428 | 0.0981 | 0.4619 | 1.5756 | 1.1791 | 2 | 1 |
| WCM162 | 12 | 2613521 | 3768751 | 0.7029 | 1.3147 | 3.5477 | 1.4275 | 4 | 1 |
| WCM162 | 12 | 3782578 | 10324618 | 0.2627 | 0.7094 | 1.6853 | 1.5849 | 2 | 2 |
| WCM162 | 12 | 10332177 | 25706018 | −0.1713 | 0.0205 | 1.0143 | 1.0143 | 1 | 1 |
| WCM162 | 12 | 25801446 | 38327906 | 0.4527 | 0.9792 | 2.1486 | 1.7943 | 2 | 2 |
| WCM162 | 12 | 38362752 | 55361593 | −0.1891 | −0.0106 | 0.9927 | 0.9927 | 1 | 1 |
| WCM162 | 12 | 55367178 | 57112802 | 0.135 | 0.5186 | 1.4326 | 1.4326 | 1 | 1 |
| WCM162 | 12 | 57113144 | 58240199 | 0.529 | 1.0836 | 2.1567 | 2.082 | 2 | 2 |
| WCM162 | 12 | 58335495 | 66537186 | −0.1249 | 0.1004 | 1.2054 | 0.9387 | 1 | 1 |
| WCM162 | 12 | 66539585 | 71519125 | 0.4479 | 0.9726 | 2.8834 | 1.0414 | 3 | 1 |
| WCM162 | 12 | 71523111 | 94727444 | −0.1462 | 0.0639 | 1.0759 | 1.0147 | 1 | 1 |
| WCM162 | 12 | 94729482 | 97222870 | 0.4391 | 0.9604 | 2.8558 | 1.0359 | 3 | 1 |
| WCM162 | 12 | 97254620 | 105198607 | −0.2192 | −0.064 | 0.9566 | 0.9566 | 1 | 1 |
| WCM162 | 12 | 105199021 | 110390335 | 0.428 | 0.9449 | 2.6513 | 1.199 | 3 | 1 |
| WCM162 | 12 | 110391038 | 116397465 | −0.2248 | −0.0741 | 0.9721 | 0.9278 | 1 | 1 |
| WCM162 | 12 | 116397584 | 120806700 | 0.3701 | 0.8637 | 1.8197 | 1.8197 | 2 | 2 |
| WCM162 | 12 | 120875914 | 123246794 | −0.3197 | −0.2492 | 0.8414 | 0.8414 | 1 | 1 |
| WCM162 | 12 | 123247491 | 124498969 | 0.5834 | 1.1569 | 3.504 | 0.9556 | 4 | 1 |
| WCM162 | 12 | 124499429 | 133810842 | −0.1093 | 0.1269 | 1.0919 | 1.0919 | 1 | 1 |
| WCM162 | 13 | 19042059 | 36822838 | 0.2653 | 0.7132 | 2.0224 | 1.2564 | 2 | 1 |
| WCM162 | 13 | 36828152 | 92380952 | −0.2485 | −0.117 | 0.9221 | 0.9221 | 1 | 1 |
| WCM162 | 13 | 92408638 | 112526718 | −0.0014 | 0.305 | 1.2413 | 1.2294 | 1 | 1 |
| WCM162 | 13 | 112527826 | 115089411 | −0.2195 | −0.0646 | 0.9562 | 0.9562 | 1 | 1 |
| WCM162 | 14 | 19110392 | 36005387 | −0.2387 | −0.0992 | 0.9336 | 0.9336 | 1 | 1 |
| WCM162 | 14 | 36005659 | 38277999 | 0.2333 | 0.6661 | 1.7132 | 1.4604 | 2 | 1 |
| WCM162 | 14 | 38281452 | 54897173 | −0.3379 | −0.2839 | 0.8214 | 0.8214 | 1 | 1 |
| WCM162 | 14 | 54898917 | 60470474 | 0.0587 | 0.4005 | 1.3531 | 1.2867 | 1 | 1 |
| WCM162 | 14 | 60470542 | 75748011 | −0.1533 | 0.0516 | 1.0364 | 1.0364 | 1 | 1 |
| WCM162 | 14 | 75748361 | 78709578 | 0.323 | 0.7967 | 1.8692 | 1.6051 | 2 | 2 |
| WCM162 | 14 | 78709776 | 94756701 | −0.135 | 0.0832 | 1.0593 | 1.0593 | 1 | 1 |
| WCM162 | 14 | 94756968 | 99880213 | 0.2111 | 0.6332 | 1.9798 | 1.1222 | 2 | 1 |
| WCM162 | 14 | 99924727 | 100615442 | 0.8247 | 1.4716 | 4.3395 | 1.207 | 4 | 1 |
| WCM162 | 14 | 100615561 | 106385364 | 0.3519 | 0.8379 | 1.8332 | 1.7417 | 2 | 2 |
| WCM162 | 14 | 106386996 | 107283255 | 0.1651 | 0.5643 | NA | NA | NA | NA |
| WCM162 | 15 | 20083896 | 51250610 | −0.1511 | 0.0556 | 1.0393 | 1.0393 | 1 | 1 |
| WCM162 | 15 | 51250944 | 54817819 | 0.2262 | 0.6557 | 1.6429 | 1.5078 | 2 | 2 |
| WCM162 | 15 | 54825172 | 75759536 | −0.1767 | 0.011 | 1.0076 | 1.0076 | 1 | 1 |
| WCM162 | 15 | 75760095 | 78346326 | 0.5481 | 1.1094 | 3.5321 | 0.7832 | 4 | 1 |
| WCM162 | 15 | 78358330 | 80464560 | −0.2089 | −0.0458 | 0.9687 | 0.9687 | 1 | 1 |
| WCM162 | 15 | 80465300 | 85191184 | 0.313 | 0.7822 | 2.1082 | 1.3313 | 2 | 1 |
| WCM162 | 15 | 85191825 | 102516227 | −0.1066 | 0.1314 | 1.0954 | 1.0954 | 1 | 1 |
| WCM162 | 16 | 64400 | 3788610 | −0.2216 | −0.0684 | 0.9537 | 0.9537 | 1 | 1 |
| WCM162 | 16 | 3789581 | 5145405 | 0.3861 | 0.8864 | 2.5331 | 1.1639 | 3 | 1 |
| WCM162 | 16 | 5147665 | 10576420 | −0.1785 | 0.008 | 1.0055 | 1.0055 | 1 | 1 |
| WCM162 | 16 | 10624302 | 13297242 | 0.4115 | 0.9219 | 2.7634 | 1.0257 | 3 | 1 |
| WCM162 | 16 | 13307046 | 18817489 | −0.1798 | 0.0055 | 1.0038 | 1.0038 | 1 | 1 |
| WCM162 | 16 | 18817730 | 18882673 | 0.0723 | 0.4218 | NA | NA | NA | NA |
| WCM162 | 16 | 18883592 | 20605121 | 0.9364 | 1.6121 | 4.917 | 1.1971 | 5 | 1 |
| WCM162 | 16 | 20634764 | 21261856 | 0.2186 | 0.6444 | NA | NA | NA | NA |
| WCM162 | 16 | 21261989 | 27789278 | −0.0844 | 0.1687 | 1.124 | 1.124 | 1 | 1 |
| WCM162 | 16 | 27789831 | 29814805 | 0.3731 | 0.868 | 2.5758 | 1.0745 | 3 | 1 |
| WCM162 | 16 | 29815328 | 30667618 | −0.0994 | 0.1436 | 1.2181 | 0.9913 | 1 | 1 |
| WCM162 | 16 | 30671691 | 32941839 | 0.3789 | 0.8761 | 1.8354 | 1.8354 | 2 | 2 |
| WCM162 | 16 | 32945341 | 65390380 | −0.1634 | 0.0341 | 1.0239 | 1.0239 | 1 | 1 |
| WCM162 | 16 | 65397073 | 67440075 | 0.3797 | 0.8773 | 1.8369 | 1.8369 | 2 | 2 |
| WCM162 | 16 | 67440339 | 70818759 | −0.1372 | 0.0793 | 1.0819 | 1.0312 | 1 | 1 |
| WCM162 | 16 | 70819754 | 71127657 | −0.3441 | −0.2959 | NA | NA | NA | NA |
| WCM162 | 16 | 71209574 | 88502662 | −0.6917 | −1.063 | 0.9657 | −0.0084 | 1 | 0 |
| WCM162 | 16 | 88502933 | 90124889 | −0.3724 | −0.3508 | 1.6411 | −0.0728 | 2 | 0 |
| WCM162 | 16 | 90125408 | 90168693 | 0.0311 | 0.3569 | NA | NA | NA | NA |
| WCM162 | 17 | 131595 | 8638800 | −0.0709 | 0.1912 | 1.1417 | 1.1417 | 1 | 1 |
| WCM162 | 17 | 8644766 | 11882027 | 0.482 | 1.0196 | 2.0274 | 2.0274 | 2 | 2 |
| WCM162 | 17 | 11886699 | 28999184 | 0.0509 | 0.3882 | 1.4037 | 1.2138 | 1 | 1 |
| WCM162 | 17 | 29059119 | 29219818 | −0.392 | −0.3895 | NA | NA | NA | NA |
| WCM162 | 17 | 29220389 | 37561613 | −0.6758 | −1.0222 | 0.9424 | 0.0423 | 1 | 0 |
| WCM162 | 17 | 37563252 | 38956108 | −0.2447 | −0.11 | 1.9637 | −0.1106 | 2 | 0 |
| WCM162 | 17 | 38974707 | 41622705 | −0.7545 | −1.2306 | 0.8866 | −0.0343 | 1 | 0 |
| WCM162 | 17 | 41623030 | 44271152 | −0.2686 | −0.1536 | 1.9109 | −0.1129 | 2 | 0 |
| WCM162 | 17 | 44788445 | 45760741 | 0.0628 | 0.4069 | NA | NA | NA | NA |
| WCM162 | 17 | 45760961 | 48072078 | 0.6638 | 1.2636 | 3.4264 | 1.3754 | 3 | 1 |
| WCM162 | 17 | 48072344 | 57350263 | 0.032 | 0.3583 | 1.2962 | 1.2677 | 1 | 1 |
| WCM162 | 17 | 57351017 | 60142547 | 0.5348 | 1.0915 | 3.1061 | 1.1557 | 3 | 1 |
| WCM162 | 17 | 60215513 | 71427648 | −0.1976 | −0.0256 | 0.9824 | 0.9824 | 1 | 1 |
| WCM162 | 17 | 71429949 | 72765215 | 0.3089 | 0.7763 | 1.957 | 1.4685 | 2 | 1 |
| WCM162 | 17 | 72768124 | 73504268 | 1.1021 | 1.8161 | 5.6034 | 1.439 | 6 | 1 |
| WCM162 | 17 | 73509765 | 81052609 | 0.0798 | 0.4335 | 1.4179 | 1.2832 | 1 | 1 |
| WCM162 | 18 | 47680 | 78005245 | −0.1088 | 0.1278 | 1.0926 | 1.0926 | 1 | 1 |
| WCM162 | 19 | 104963 | 362418 | −0.3526 | −0.3122 | NA | NA | NA | NA |
| WCM162 | 19 | 367100 | 3281149 | 0.2067 | 0.6268 | 1.5947 | 1.4936 | 2 | 1 |
| WCM162 | 19 | 3282053 | 13319842 | −0.1881 | −0.0089 | 0.9939 | 0.9939 | 1 | 1 |
| WCM162 | 19 | 13321452 | 16136395 | 0.1024 | 0.4686 | 1.3837 | 1.3837 | 1 | 1 |
| WCM162 | 19 | 16136636 | 42930955 | −0.1939 | −0.0191 | 0.9869 | 0.9869 | 1 | 1 |
| WCM162 | 19 | 42931202 | 45397249 | 0.2654 | 0.7133 | 1.9795 | 1.2996 | 2 | 1 |
| WCM162 | 19 | 45404020 | 59093645 | −0.2074 | −0.0432 | 0.9705 | 0.9705 | 1 | 1 |
| WCM162 | 2 | 41474 | 1079242 | −0.1624 | 0.036 | 1.3011 | 0.7494 | 1 | 1 |
| WCM162 | 2 | 1079396 | 14774018 | 0.2321 | 0.6644 | 1.6714 | 1.4983 | 2 | 1 |
| WCM162 | 2 | 14774374 | 25194741 | −0.1889 | −0.0103 | 0.9929 | 0.9929 | 1 | 1 |
| WCM162 | 2 | 25306119 | 26818100 | 0.1357 | 0.5198 | 1.5716 | 1.2959 | 2 | 1 |
| WCM162 | 2 | 26828459 | 29136874 | −0.2856 | −0.1851 | 0.8796 | 0.8796 | 1 | 1 |
| WCM162 | 2 | 29137129 | 36774339 | 0.1014 | 0.4671 | 1.44 | 1.3247 | 1 | 1 |
| WCM162 | 2 | 36775661 | 51259378 | −0.2516 | −0.1226 | 0.9185 | 0.9185 | 1 | 1 |
| WCM162 | 2 | 51662228 | 65540794 | 0.207 | 0.6273 | 1.8274 | 1.2618 | 2 | 1 |
| WCM162 | 2 | 65540905 | 86831060 | −0.2083 | −0.0447 | 0.9695 | 0.9695 | 1 | 1 |
| WCM162 | 2 | 86831148 | 103433142 | −0.0361 | 0.2486 | 1.223 | 1.1531 | 1 | 1 |
| WCM162 | 2 | 105185363 | 112944976 | 0.1887 | 0.5998 | 1.5791 | 1.452 | 2 | 1 |
| WCM162 | 2 | 112988488 | 138722273 | −0.1564 | 0.0463 | 1.0326 | 1.0326 | 1 | 1 |
| WCM162 | 2 | 138724524 | 144525805 | 0.4922 | 1.0335 | 2.4252 | 1.6688 | 2 | 2 |
| WCM162 | 2 | 144699787 | 169551552 | −0.0479 | 0.2292 | 1.2049 | 1.1394 | 1 | 1 |
| WCM162 | 2 | 169571466 | 170428398 | 0.3899 | 0.8916 | 2.3641 | 1.3464 | 2 | 1 |
| WCM162 | 2 | 170440959 | 207621974 | −0.0657 | 0.1999 | 1.1486 | 1.1486 | 1 | 1 |
| WCM162 | 2 | 207622091 | 216300375 | −0.2166 | −0.0595 | 0.9596 | 0.9596 | 1 | 1 |
| WCM162 | 2 | 216743547 | 219942790 | 0.349 | 0.8337 | 2.5255 | 1.039 | 3 | 1 |
| WCM162 | 2 | 219959961 | 232327894 | −0.1547 | 0.0492 | 1.0347 | 1.0347 | 1 | 1 |
| WCM162 | 2 | 232329052 | 234741227 | 0.1433 | 0.5314 | 1.5575 | 1.3331 | 2 | 1 |
| WCM162 | 2 | 234741739 | 243052762 | −0.2043 | −0.0375 | 0.9743 | 0.9743 | 1 | 1 |
| WCM162 | 20 | 68312 | 6760367 | −0.186 | −0.0052 | 0.9964 | 0.9964 | 1 | 1 |
| WCM162 | 20 | 6760770 | 14066276 | 0.4127 | 0.9236 | 2.5226 | 1.2712 | 3 | 1 |
| WCM162 | 20 | 14066797 | 46307567 | −0.1821 | 0.0016 | 1.0011 | 1.0011 | 1 | 1 |
| WCM162 | 20 | 46311866 | 55749953 | 0.1226 | 0.4996 | 1.4963 | 1.3315 | 1 | 1 |
| WCM162 | 20 | 55758827 | 58441500 | 0.613 | 1.1965 | 2.2918 | 2.2918 | 2 | 2 |
| WCM162 | 20 | 58441609 | 60808435 | 0.0596 | 0.4019 | 1.56 | 1.0826 | 2 | 1 |
| WCM162 | 20 | 60831247 | 62934623 | −0.1902 | −0.0127 | 0.9912 | 0.9912 | 1 | 1 |
| WCM162 | 21 | 9590271 | 45008625 | −0.1962 | −0.0232 | 0.984 | 0.984 | 1 | 1 |
| WCM162 | 21 | 45012148 | 47545534 | 0.1643 | 0.5632 | 1.578 | 1.3771 | 2 | 1 |
| WCM162 | 21 | 47545922 | 48084175 | −0.3315 | −0.2716 | 0.8284 | 0.8284 | 1 | 1 |
| WCM162 | 22 | 16159060 | 24181169 | 0.3801 | 0.8779 | 2.3691 | 1.3063 | 2 | 1 |
| WCM162 | 22 | 24199151 | 40859315 | −0.2087 | −0.0453 | 0.9691 | 0.9691 | 1 | 1 |
| WCM162 | 22 | 40915494 | 49146061 | 0.0994 | 0.464 | 1.3793 | 1.3793 | 1 | 1 |
| WCM162 | 22 | 49146347 | 51219090 | −0.3264 | −0.2618 | 0.834 | 0.834 | 1 | 1 |
| WCM162 | 3 | 239524 | 9990294 | −0.1145 | 0.1181 | 1.0853 | 1.0853 | 1 | 1 |
| WCM162 | 3 | 9991041 | 13860590 | 0.386 | 0.8862 | 2.4104 | 1.2861 | 2 | 1 |
| WCM162 | 3 | 13860873 | 38042439 | −0.079 | 0.1778 | 1.1311 | 1.1311 | 1 | 1 |
| WCM162 | 3 | 38042850 | 38991885 | 0.4486 | 0.9735 | 2.7518 | 1.1754 | 3 | 1 |
| WCM162 | 3 | 39093406 | 47870459 | −0.1642 | 0.0329 | 1.023 | 1.023 | 1 | 1 |
| WCM162 | 3 | 47882481 | 49753788 | 0.2944 | 0.7554 | 1.7541 | 1.6222 | 2 | 2 |
| WCM162 | 3 | 49755498 | 50683265 | −0.2364 | −0.0951 | NA | NA | NA | NA |
| WCM162 | 3 | 50683692 | 54352241 | 0.1352 | 0.519 | 1.5379 | 1.3279 | 2 | 1 |
| WCM162 | 3 | 54354431 | 63953527 | −0.1493 | 0.0587 | 1.0415 | 1.0415 | 1 | 1 |
| WCM162 | 3 | 63965712 | 67568710 | 0.3416 | 0.8232 | 2.7233 | 0.8154 | 3 | 1 |
| WCM162 | 3 | 67570888 | 112301532 | −0.204 | −0.0371 | 0.9746 | 0.9746 | 1 | 1 |
| WCM162 | 3 | 112324305 | 133914095 | 0.1718 | 0.5745 | 1.7853 | 1.193 | 2 | 1 |
| WCM162 | 3 | 133921683 | 197896724 | −0.0989 | 0.1445 | 1.1053 | 1.1053 | 1 | 1 |
| WCM162 | 4 | 53423 | 15055789 | −0.0408 | 0.2408 | 1.1817 | 1.1817 | 1 | 1 |
| WCM162 | 4 | 15059988 | 17802412 | 0.4506 | 0.9763 | 2.9173 | 1.0175 | 3 | 1 |
| WCM162 | 4 | 17803748 | 73148843 | 0.0342 | 0.3619 | 1.2994 | 1.2708 | 1 | 1 |
| WCM162 | 4 | 73149313 | 76434534 | 0.4143 | 0.9258 | 2.205 | 1.5945 | 2 | 2 |
| WCM162 | 4 | 76439485 | 106638763 | −0.1734 | 0.0168 | 1.0117 | 1.0117 | 1 | 1 |
| WCM162 | 4 | 106639102 | 113726300 | 0.2546 | 0.6975 | 1.9116 | 1.3318 | 2 | 1 |
| WCM162 | 4 | 113728100 | 143029296 | −0.1458 | 0.0647 | 1.0459 | 1.0459 | 1 | 1 |
| WCM162 | 4 | 143033676 | 149357758 | 0.2187 | 0.6446 | 1.7324 | 1.3941 | 2 | 1 |
| WCM162 | 4 | 149358014 | 166389045 | −0.1847 | −0.003 | 0.9979 | 0.9979 | 1 | 1 |
| WCM162 | 4 | 166403455 | 173704113 | 0.5416 | 1.1008 | 2.7129 | 1.5765 | 3 | 2 |
| WCM162 | 4 | 173730564 | 190903980 | −0.0853 | 0.1673 | 1.123 | 1.123 | 1 | 1 |
| WCM162 | 5 | 90696 | 5469124 | −0.1437 | 0.0683 | 1.0485 | 1.0485 | 1 | 1 |
| WCM162 | 5 | 5473728 | 41842746 | 0.0228 | 0.3436 | 1.2689 | 1.2689 | 1 | 1 |
| WCM162 | 5 | 41850204 | 96512820 | −0.2353 | −0.093 | 0.9375 | 0.9375 | 1 | 1 |
| WCM162 | 5 | 96513453 | 109025747 | −0.6421 | −0.9383 | 1.0566 | −0.0129 | 1 | 0 |
| WCM162 | 5 | 109026194 | 109203296 | −0.0536 | 0.2199 | NA | NA | NA | NA |
| WCM162 | 5 | 109668614 | 114860322 | 0.5379 | 1.0957 | 2.1372 | 2.1372 | 2 | 2 |
| WCM162 | 5 | 114860647 | 135469500 | −0.1827 | 5.00E−04 | 1.0004 | 1.0004 | 1 | 1 |
| WCM162 | 5 | 135469770 | 137893545 | 0.5592 | 1.1244 | 3.0165 | 1.3438 | 3 | 1 |
| WCM162 | 5 | 137894341 | 140795145 | −0.3669 | −0.34 | 0.79 | 0.79 | 1 | 1 |
| WCM162 | 5 | 140797493 | 161324532 | −0.0676 | 0.1968 | 1.1461 | 1.1461 | 1 | 1 |
| WCM162 | 5 | 161325273 | 167182213 | −0.299 | −0.21 | 0.8645 | 0.8645 | 1 | 1 |
| WCM162 | 5 | 167182240 | 169714998 | 0.4445 | 0.9678 | 1.9559 | 1.9559 | 2 | 2 |
| WCM162 | 5 | 169720203 | 180687847 | −0.1809 | 0.0037 | 1.0026 | 1.0026 | 1 | 1 |
| WCM162 | 6 | 203561 | 40996318 | −0.1892 | −0.0108 | 0.9925 | 0.9925 | 1 | 1 |
| WCM162 | 6 | 40998169 | 46135842 | 0.4062 | 0.9144 | 1.8848 | 1.8848 | 2 | 2 |
| WCM162 | 6 | 46188487 | 73112380 | −0.1425 | 0.0704 | 1.05 | 1.05 | 1 | 1 |
| WCM162 | 6 | 73248242 | 86181051 | 0.1073 | 0.4762 | 1.4898 | 1.2924 | 1 | 1 |
| WCM162 | 6 | 86181214 | 99322151 | −0.2454 | −0.1112 | 0.9258 | 0.9258 | 1 | 1 |
| WCM162 | 6 | 99323323 | 102012757 | 0.7225 | 1.3402 | 3.9582 | 1.1057 | 4 | 1 |
| WCM162 | 6 | 102069937 | 128841423 | −0.1902 | −0.0126 | 0.9913 | 0.9913 | 1 | 1 |
| WCM162 | 6 | 129204447 | 134758977 | −0.001 | 0.3056 | 1.5404 | 0.9314 | 2 | 1 |
| WCM162 | 6 | 134785989 | 138763072 | 0.4518 | 0.9779 | 2.193 | 1.7462 | 2 | 2 |
| WCM162 | 6 | 138768206 | 159187640 | 0.0625 | 0.4065 | 1.3255 | 1.3255 | 1 | 1 |
| WCM162 | 6 | 159187941 | 170892848 | −0.1688 | 0.0249 | 1.0174 | 1.0174 | 1 | 1 |
| WCM162 | 7 | 193189 | 2834585 | −0.107 | 0.1308 | 1.0949 | 1.0949 | 1 | 1 |
| WCM162 | 7 | 2839721 | 28547407 | 0.211 | 0.6331 | 1.6224 | 1.4794 | 2 | 1 |
| WCM162 | 7 | 28552418 | 32527304 | 0.7761 | 1.4095 | 2.7178 | 2.595 | 3 | 3 |
| WCM162 | 7 | 32528216 | 48634325 | 0.3817 | 0.8801 | 2.536 | 1.1451 | 3 | 1 |
| WCM162 | 7 | 48634484 | 65705605 | 0.0669 | 0.4134 | 1.3318 | 1.3318 | 1 | 1 |
| WCM162 | 7 | 65705779 | 79828710 | 0.3794 | 0.8768 | 1.9943 | 1.6783 | 2 | 2 |
| WCM162 | 7 | 79828753 | 92361119 | −0.2407 | −0.1028 | 0.9312 | 0.9312 | 1 | 1 |
| WCM162 | 7 | 92367215 | 95216390 | 0.2253 | 0.6544 | 1.858 | 1.2899 | 2 | 1 |
| WCM162 | 7 | 95216684 | 96765730 | −0.1445 | 0.067 | NA | NA | NA | NA |
| WCM162 | 7 | 96810324 | 100088698 | 0.7505 | 1.3765 | 4.0639 | 1.1287 | 4 | 1 |
| WCM162 | 7 | 100092412 | 108524326 | −0.2393 | −0.1002 | 0.9717 | 0.8941 | 1 | 1 |
| WCM162 | 7 | 108524593 | 120737809 | 0.1082 | 0.4776 | 1.4504 | 1.3344 | 1 | 1 |
| WCM162 | 7 | 120739990 | 122001002 | 0.5855 | 1.1597 | 3.1815 | 1.2868 | 3 | 1 |
| WCM162 | 7 | 122001089 | 141920843 | −0.04 | 0.2422 | 1.1828 | 1.1828 | 1 | 1 |
| WCM162 | 7 | 141920981 | 146536869 | 0.5458 | 1.1064 | 3.2637 | 1.0424 | 3 | 1 |
| WCM162 | 7 | 146590712 | 158828679 | 0.0611 | 0.4043 | 1.3608 | 1.286 | 1 | 1 |
| WCM162 | 7 | 158829410 | 158937158 | −0.2861 | −0.1861 | NA | NA | NA | NA |
| WCM162 | 8 | 163461 | 12435678 | −0.5474 | −0.7169 | 1.1372 | 0.0796 | 1 | 0 |
| WCM162 | 8 | 12493711 | 21549666 | −0.2803 | −0.1754 | 1.8121 | −0.041 | 2 | 0 |
| WCM162 | 8 | 21549713 | 30610655 | −0.6642 | −0.993 | 0.9989 | 0.006 | 1 | 0 |
| WCM162 | 8 | 30612148 | 68982167 | −0.1886 | −0.0098 | 0.9932 | 0.9932 | 1 | 1 |
| WCM162 | 8 | 68984721 | 74650536 | 0.4807 | 1.0178 | 2.5917 | 1.4578 | 3 | 1 |
| WCM162 | 8 | 74692722 | 102644013 | −0.1698 | 0.0231 | 1.0162 | 1.0162 | 1 | 1 |
| WCM162 | 8 | 102644486 | 144644249 | 0.1987 | 0.6148 | 1.6113 | 1.4513 | 2 | 1 |
| WCM162 | 8 | 144644623 | 145732226 | 0.6201 | 1.2058 | 3.5372 | 1.0761 | 4 | 1 |
| WCM162 | 8 | 145732395 | 145979138 | 0.1523 | 0.5451 | 1.6672 | 1.251 | 2 | 1 |
| WCM162 | 8 | 145979779 | 146279371 | −0.1125 | 0.1215 | 1.1303 | 1.0455 | 1 | 1 |
| WCM162 | 9 | 15883 | 711396 | 0.1166 | 0.4905 | 1.4892 | 1.3207 | 1 | 1 |
| WCM162 | 9 | 711743 | 78631328 | 0.3668 | 0.8591 | 1.9089 | 1.7189 | 2 | 2 |
| WCM162 | 9 | 78638689 | 88457704 | −0.0839 | 0.1696 | 1.1247 | 1.1247 | 1 | 1 |
| WCM162 | 9 | 88460884 | 93637188 | 0.228 | 0.6584 | 2.0939 | 1.0628 | 2 | 1 |
| WCM162 | 9 | 93639973 | 95477576 | 0.7243 | 1.3426 | 2.7433 | 2.3288 | 3 | 2 |
| WCM162 | 9 | 95477693 | 97356033 | 0.1502 | 0.5417 | 1.5684 | 1.343 | 2 | 1 |
| WCM162 | 9 | 97365642 | 100088893 | −0.1237 | 0.1025 | 1.0736 | 1.0736 | 1 | 1 |
| WCM162 | 9 | 100090254 | 101915916 | 0.3979 | 0.9029 | 1.9643 | 1.7754 | 2 | 2 |
| WCM162 | 9 | 101979938 | 124241532 | −0.142 | 0.0711 | 1.0505 | 1.0505 | 1 | 1 |
| WCM162 | 9 | 124329532 | 131831408 | 0.2007 | 0.6178 | 1.6559 | 1.4133 | 2 | 1 |
| WCM162 | 9 | 131831971 | 141107532 | −0.1224 | 0.1046 | 1.0752 | 1.0752 | 1 | 1 |
| WCM162 | X | 2700189 | 43817834 | −0.1442 | 0.0674 | NA | NA | NA | NA |
| WCM162 | X | 44008014 | 49593896 | 1.0019 | 1.6934 | NA | NA | NA | NA |
| WCM162 | X | 49595103 | 64754601 | 0.2881 | 0.7463 | NA | NA | NA | NA |
| WCM162 | X | 64887665 | 68840787 | 1.0303 | 1.7284 | NA | NA | NA | NA |
| WCM162 | X | 68844800 | 154493590 | −0.0569 | 0.2144 | NA | NA | NA | NA |
| WCM162_Z1_1 | 1 | 721450 | 1007837 | −0.2375 | 0.1023 | NA | NA | NA | NA |
| WCM162_Z1_1 | 1 | 1018144 | 1670405 | −0.9737 | −0.845 | 1.0784 | 0.0351 | 1 | 0 |
| WCM162_Z1_1 | 1 | 1684475 | 3703501 | 0.2567 | 0.6796 | 1.9241 | 1.2794 | 2 | 1 |
| WCM162_Z1_1 | 1 | 3703695 | 11595551 | −0.2764 | 0.0553 | 1.0618 | 1.0164 | 1 | 1 |
| WCM162_Z1_1 | 1 | 11596396 | 14220186 | 0.1632 | 0.5729 | 2.0235 | 0.9515 | 2 | 1 |
| WCM162_Z1_1 | 1 | 14506932 | 17312660 | −0.3123 | 0.0117 | 1.1616 | 0.8547 | 1 | 1 |
| WCM162_Z1_1 | 1 | 17312787 | 20678580 | 0.1543 | 0.5627 | 1.9164 | 1.0376 | 2 | 1 |
| WCM162_Z1_1 | 1 | 20680293 | 21231381 | −0.2631 | 0.0715 | NA | NA | NA | NA |
| WCM162_Z1_1 | 1 | 21267821 | 24076429 | −1.0632 | −0.9714 | 1.009 | 0.011 | 1 | 0 |
| WCM162_Z1_1 | 1 | 24077432 | 28907543 | −0.3079 | 0.0171 | 1.0341 | 0.9897 | 1 | 1 |
| WCM162_Z1_1 | 1 | 28919190 | 31187101 | 0.7898 | 1.2726 | NA | NA | NA | NA |
| WCM162_Z1_1 | 1 | 31188089 | 61869783 | −0.2998 | 0.027 | 1.035 | 1.0028 | 1 | 1 |
| WCM162_Z1_1 | 1 | 61872311 | 63991267 | 0.339 | 0.7729 | 2.0944 | 1.3231 | 2 | 1 |
| WCM162_Z1_1 | 1 | 63997633 | 65351941 | −0.3076 | 0.0174 | NA | NA | NA | NA |
| WCM162_Z1_1 | 1 | 65523519 | 70687455 | 0.3745 | 0.8129 | 2.2942 | 1.2192 | 2 | 1 |
| WCM162_Z1_1 | 1 | 70694111 | 75608851 | −0.2838 | 0.0464 | 1.1053 | 0.96 | 1 | 1 |
| WCM162_Z1_1 | 1 | 75609547 | 76081549 | 0.1834 | 0.596 | NA | NA | NA | NA |
| WCM162_Z1_1 | 1 | 76190494 | 78585145 | 0.6757 | 1.1476 | NA | NA | NA | NA |
| WCM162_Z1_1 | 1 | 78591638 | 87045830 | 0.2057 | 0.6215 | 2.1696 | 0.9074 | 2 | 1 |
| WCM162_Z1_1 | 1 | 87100046 | 91182689 | −0.3028 | 0.0233 | 1.1077 | 0.9248 | 1 | 1 |
| WCM162_Z1_1 | 1 | 91382424 | 92528654 | 0.9669 | 1.4648 | NA | NA | NA | NA |
| WCM162_Z1_1 | 1 | 92546144 | 100111889 | −0.3287 | −0.0082 | 1.0368 | 0.9518 | 1 | 1 |
| WCM162_Z1_1 | 1 | 100127889 | 110921508 | 0.1703 | 0.581 | 1.9367 | 1.055 | 2 | 1 |
| WCM162_Z1_1 | 1 | 110921797 | 113933947 | −0.2964 | 0.0311 | 1.0925 | 0.951 | 1 | 1 |
| WCM162_Z1_1 | 1 | 114092219 | 115537613 | 0.1866 | 0.5996 | NA | NA | NA | NA |
| WCM162_Z1_1 | 1 | 115576068 | 145326086 | −0.3065 | 0.0188 | 1.2867 | 0.7395 | 1 | 1 |
| WCM162_Z1_1 | 1 | 145367178 | 149877531 | 0.3357 | 0.7692 | NA | NA | NA | NA |
| WCM162_Z1_1 | 1 | 149878317 | 152052703 | 0.6575 | 1.1276 | NA | NA | NA | NA |
| WCM162_Z1_1 | 1 | 152052722 | 152323132 | 0.2918 | 0.7195 | 2.2307 | 1.0625 | 2 | 1 |
| WCM162_Z1_1 | 1 | 152323409 | 153752448 | −0.2872 | 0.0422 | 1.0997 | 0.9597 | 1 | 1 |
| WCM162_Z1_1 | 1 | 153902813 | 155319144 | 0.1556 | 0.5642 | 1.9285 | 1.0285 | 2 | 1 |
| WCM162_Z1_1 | 1 | 155319384 | 155942053 | 0.4995 | 0.9528 | NA | NA | NA | NA |
| WCM162_Z1_1 | 1 | 155948165 | 157963481 | −0.3247 | −0.0034 | 1.0354 | 0.9599 | 1 | 1 |
| WCM162_Z1_1 | 1 | 158045960 | 159002453 | 0.6429 | 1.1116 | 3.3866 | 0.9349 | 3 | 1 |
| WCM162_Z1_1 | 1 | 159015238 | 160313230 | 0.9693 | 1.4673 | 4.2097 | 1.3206 | 4 | 1 |
| WCM162_Z1_1 | 1 | 160313565 | 162769689 | 0.6558 | 1.1258 | 3.2413 | 1.123 | 3 | 1 |
| WCM162_Z1_1 | 1 | 162773300 | 163325157 | 0.1554 | 0.5639 | NA | NA | NA | NA |
| WCM162_Z1_1 | 1 | 164529115 | 164818608 | −0.4865 | −0.2033 | NA | NA | NA | NA |
| WCM162_Z1_1 | 1 | 164853793 | 167734968 | −0.9736 | −0.8449 | NA | NA | NA | NA |
| WCM162_Z1_1 | 1 | 167741559 | 169857919 | −0.2916 | 0.0369 | 2.0584 | −0.0065 | 2 | 0 |
| WCM162_Z1_1 | 1 | 169890853 | 182027035 | −1.0302 | −0.9244 | 1.0434 | 0.0104 | 1 | 0 |
| WCM162_Z1_1 | 1 | 182353729 | 184023947 | −0.2738 | 0.0585 | 2.1295 | −0.0468 | 2 | 0 |
| WCM162_Z1_1 | 1 | 184041389 | 185704020 | 0.6979 | 1.172 | NA | NA | NA | NA |
| WCM162_Z1_1 | 1 | 185704178 | 190423920 | −0.2548 | 0.0815 | 1.0901 | 1.0262 | 1 | 1 |
| WCM162_Z1_1 | 1 | 192127815 | 193218871 | 0.5973 | 1.0612 | NA | NA | NA | NA |
| WCM162_Z1_1 | 1 | 193219820 | 202152786 | 0.2026 | 0.6179 | 2.0346 | 1.0347 | 2 | 1 |
| WCM162_Z1_1 | 1 | 202155661 | 203139575 | −0.2898 | 0.0391 | NA | NA | NA | NA |
| WCM162_Z1_1 | 1 | 203140225 | 204135389 | 0.1916 | 0.6054 | NA | NA | NA | NA |
| WCM162_Z1_1 | 1 | 204159844 | 204966684 | 1.0563 | 1.561 | NA | NA | NA | NA |
| WCM162_Z1_1 | 1 | 204970302 | 207110638 | 0.6629 | 1.1336 | 3.4443 | 0.9438 | 3 | 1 |
| WCM162_Z1_1 | 1 | 207110936 | 207532982 | 0.9344 | 1.4297 | NA | NA | NA | NA |
| WCM162_Z1_1 | 1 | 207627820 | 209602666 | −0.301 | 0.0255 | 1.0327 | 1.003 | 1 | 1 |
| WCM162_Z1_1 | 1 | 209603784 | 212788501 | 0.4091 | 0.8517 | 2.2965 | 1.3128 | 2 | 1 |
| WCM162_Z1_1 | 1 | 212791525 | 221057662 | −0.2694 | 0.0638 | 1.0907 | 0.9997 | 1 | 1 |
| WCM162_Z1_1 | 1 | 221057972 | 228699923 | 0.0795 | 0.4764 | 1.649 | 1.1335 | 2 | 1 |
| WCM162_Z1_1 | 1 | 228786094 | 238053863 | −0.2807 | 0.0501 | 1.0655 | 1.0052 | 1 | 1 |
| WCM162_Z1_1 | 1 | 240070851 | 242035574 | 0.1552 | 0.5636 | 1.9227 | 1.0332 | 2 | 1 |
| WCM162_Z1_1 | 1 | 242042206 | 245180554 | 0.6297 | 1.097 | NA | NA | NA | NA |
| WCM162_Z1_1 | 1 | 245222729 | 246670511 | 0.1851 | 0.598 | NA | NA | NA | NA |
| WCM162_Z1_1 | 1 | 246704458 | 249212550 | −0.3197 | 0.0028 | 1.0215 | 0.9824 | 1 | 1 |
| WCM162_Z1_1 | 10 | 225960 | 3827174 | −0.3258 | −0.0048 | 1.0903 | 0.903 | 1 | 1 |
| WCM162_Z1_1 | 10 | 4867218 | 6557025 | 0.1559 | 0.5644 | 1.8949 | 1.0627 | 2 | 1 |
| WCM162_Z1_1 | 10 | 7205773 | 23326228 | −0.231 | 0.1102 | 1.1105 | 1.0482 | 1 | 1 |
| WCM162_Z1_1 | 10 | 23393134 | 25877958 | 0.1543 | 0.5626 | NA | NA | NA | NA |
| WCM162_Z1_1 | 10 | 25883204 | 28884783 | −0.2327 | 0.1081 | 1.144 | 1.0117 | 1 | 1 |
| WCM162_Z1_1 | 10 | 28897226 | 33623304 | 0.3033 | 0.7325 | 2.2766 | 1.0464 | 2 | 1 |
| WCM162_Z1_1 | 10 | 34400305 | 43088922 | −0.3176 | 0.0053 | 1.1455 | 0.8618 | 1 | 1 |
| WCM162_Z1_1 | 10 | 43089209 | 54531361 | −1.0433 | −0.943 | 1.0276 | 0.0127 | 1 | 0 |
| WCM162_Z1_1 | 10 | 55566501 | 69407257 | −0.1049 | 0.2606 | 1.2291 | 1.1668 | 1 | 1 |
| WCM162_Z1_1 | 10 | 69556964 | 71392557 | 0.4322 | 0.8776 | 2.6046 | 1.0702 | 3 | 1 |
| WCM162_Z1_1 | 10 | 71392808 | 74702483 | −0.2706 | 0.0624 | 1.0922 | 0.9961 | 1 | 1 |
| WCM162_Z1_1 | 10 | 74714366 | 74884074 | 0.2801 | 0.7062 | NA | NA | NA | NA |
| WCM162_Z1_1 | 10 | 74884984 | 79784372 | 0.0605 | 0.4544 | NA | NA | NA | NA |
| WCM162_Z1_1 | 10 | 79784814 | 83637721 | 0.4902 | 0.9424 | 2.9966 | 0.8468 | 3 | 1 |
| WCM162_Z1_1 | 10 | 83926519 | 91533797 | −0.2199 | 0.1235 | 1.1444 | 1.0344 | 1 | 1 |
| WCM162_Z1_1 | 10 | 92503357 | 95216670 | 0.2739 | 0.6992 | NA | NA | NA | NA |
| WCM162_Z1_1 | 10 | 95237963 | 97810047 | −0.329 | −0.0086 | 1.0341 | 0.954 | 1 | 1 |
| WCM162_Z1_1 | 10 | 97816950 | 99024633 | 0.9379 | 1.4334 | NA | NA | NA | NA |
| WCM162_Z1_1 | 10 | 99025716 | 99968470 | 1.2695 | 1.7889 | 5.936 | 0.9748 | 6 | 1 |
| WCM162_Z1_1 | 10 | 99968673 | 100195117 | 0.8512 | 1.3395 | NA | NA | NA | NA |
| WCM162_Z1_1 | 10 | 100195405 | 102306918 | 0.5334 | 0.9905 | 2.031 | 1.9427 | 2 | 2 |
| WCM162_Z1_1 | 10 | 102307850 | 104866362 | −0.2986 | 0.0284 | NA | NA | NA | NA |
| WCM162_Z1_1 | 10 | 104899207 | 107023082 | 0.1946 | 0.6088 | 1.975 | 1.0749 | 2 | 1 |
| WCM162_Z1_1 | 10 | 108337198 | 120907359 | −0.9835 | −0.8586 | 1.0858 | 0.0172 | 1 | 0 |
| WCM162_Z1_1 | 10 | 120914657 | 123233909 | −0.0556 | 0.3188 | NA | NA | NA | NA |
| WCM162_Z1_1 | 10 | 123239130 | 128076689 | 0.2623 | 0.686 | 2.161 | 1.0567 | 2 | 1 |
| WCM162_Z1_1 | 10 | 128114649 | 131977604 | 0.7715 | 1.2526 | 3.2545 | 1.511 | 3 | 2 |
| WCM162_Z1_1 | 10 | 132760893 | 134751028 | 0.2433 | 0.6644 | 1.8881 | 1.2818 | 2 | 1 |
| WCM162_Z1_1 | 10 | 134752203 | 135440090 | −0.312 | 0.0121 | 1.0428 | 0.974 | 1 | 1 |
| WCM162_Z1_1 | 11 | 130546 | 694936 | −0.3459 | −0.0292 | 1.0135 | 0.9464 | 1 | 1 |
| WCM162_Z1_1 | 11 | 695842 | 3069028 | 0.1583 | 0.5672 | 1.9681 | 0.9951 | 2 | 1 |
| WCM162_Z1_1 | 11 | 3076167 | 9187407 | −0.3393 | −0.0212 | 1.0047 | 0.9662 | 1 | 1 |
| WCM162_Z1_1 | 11 | 9191494 | 12023977 | 0.3059 | 0.7354 | NA | NA | NA | NA |
| WCM162_Z1_1 | 11 | 12029923 | 14004426 | −0.2884 | 0.0407 | NA | NA | NA | NA |
| WCM162_Z1_1 | 11 | 14063140 | 18124802 | 0.2861 | 0.7131 | 1.9813 | 1.2973 | 2 | 1 |
| WCM162_Z1_1 | 11 | 18127581 | 42262610 | −0.2172 | 0.1267 | 1.1324 | 1.0511 | 1 | 1 |
| WCM162_Z1_1 | 11 | 43333722 | 46419157 | 0.1772 | 0.5889 | 1.9315 | 1.0766 | 2 | 1 |
| WCM162_Z1_1 | 11 | 46419299 | 47786875 | −0.3023 | 0.0239 | 1.0773 | 0.9562 | 1 | 1 |
| WCM162_Z1_1 | 11 | 47788806 | 50003964 | 0.171 | 0.5818 | 1.7576 | 1.2359 | 2 | 1 |
| WCM162_Z1_1 | 11 | 51411495 | 57583447 | −0.3335 | −0.0141 | 1.0157 | 0.9649 | 1 | 1 |
| WCM162_Z1_1 | 11 | 57798493 | 59712725 | 0.243 | 0.664 | 2.1437 | 1.0252 | 2 | 1 |
| WCM162_Z1_1 | 11 | 59807988 | 65746353 | −0.3158 | 0.0075 | 1.0284 | 0.9819 | 1 | 1 |
| WCM162_Z1_1 | 11 | 65766151 | 67012772 | 0.3948 | 0.8357 | 2.5664 | 1.0031 | 3 | 1 |
| WCM162_Z1_1 | 11 | 67013459 | 67079325 | 0.8782 | 1.3687 | NA | NA | NA | NA |
| WCM162_Z1_1 | 11 | 67119449 | 67431185 | 1.294 | 1.8149 | 6.0826 | 0.9542 | 6 | 1 |
| WCM162_Z1_1 | 11 | 67431998 | 68483386 | 0.8952 | 1.3872 | NA | NA | NA | NA |
| WCM162_Z1_1 | 11 | 68506201 | 70017016 | 0.3097 | 0.7397 | 2.3236 | 1.0161 | 2 | 1 |
| WCM162_Z1_1 | 11 | 70026131 | 94583354 | −0.2656 | 0.0684 | 1.0675 | 1.0295 | 1 | 1 |
| WCM162_Z1_1 | 11 | 94587241 | 99690428 | 0.2048 | 0.6205 | NA | NA | NA | NA |
| WCM162_Z1_1 | 11 | 99715699 | 113684612 | −0.2194 | 0.124 | 1.1199 | 1.0597 | 1 | 1 |
| WCM162_Z1_1 | 11 | 113685956 | 115102086 | −1.0571 | −0.9627 | NA | NA | NA | NA |
| WCM162_Z1_1 | 11 | 115109260 | 121500245 | −0.3056 | 0.0199 | 1.0262 | 1.0015 | 1 | 1 |
| WCM162_Z1_1 | 11 | 121970500 | 124746180 | 0.1387 | 0.5447 | 1.9152 | 1.0022 | 2 | 1 |
| WCM162_Z1_1 | 11 | 124747183 | 129321221 | −0.2668 | 0.067 | 1.083 | 1.012 | 1 | 1 |
| WCM162_Z1_1 | 11 | 129722553 | 134856611 | 0.1296 | 0.5342 | 1.9251 | 0.9712 | 2 | 1 |
| WCM162_Z1_1 | 12 | 148673 | 977409 | −0.2331 | 0.1076 | NA | NA | NA | NA |
| WCM162_Z1_1 | 12 | 977723 | 990165 | 0.4113 | 0.8542 | NA | NA | NA | NA |
| WCM162_Z1_1 | 12 | 990912 | 3659185 | 0.619 | 1.0852 | 3.3953 | 0.8481 | 3 | 1 |
| WCM162_Z1_1 | 12 | 3661961 | 6729677 | 0.4819 | 0.9332 | 2.1905 | 1.6285 | 2 | 2 |
| WCM162_Z1_1 | 12 | 6730054 | 7309199 | 0.1652 | 0.5751 | 2.0214 | 0.9582 | 2 | 1 |
| WCM162_Z1_1 | 12 | 7309471 | 8693390 | −0.3 | 0.0266 | 1.1004 | 0.9369 | 1 | 1 |
| WCM162_Z1_1 | 12 | 8756901 | 10332192 | 0.1862 | 0.5992 | 1.9849 | 1.0449 | 2 | 1 |
| WCM162_Z1_1 | 12 | 10335159 | 26026977 | −0.2182 | 0.1255 | 1.1066 | 1.0751 | 1 | 1 |
| WCM162_Z1_1 | 12 | 26208297 | 26277693 | 0.4701 | 0.9199 | NA | NA | NA | NA |
| WCM162_Z1_1 | 12 | 26348806 | 27934122 | 0.8818 | 1.3727 | 4.2777 | 0.9013 | 4 | 1 |
| WCM162_Z1_1 | 12 | 27944736 | 31286878 | 0.1684 | 0.5788 | 1.891 | 1.0962 | 2 | 1 |
| WCM162_Z1_1 | 12 | 31297871 | 32975538 | 0.6808 | 1.1533 | NA | NA | NA | NA |
| WCM162_Z1_1 | 12 | 32976997 | 34182560 | 0.2163 | 0.6337 | NA | NA | NA | NA |
| WCM162_Z1_1 | 12 | 38710697 | 43769276 | −0.3221 | −3.00E−04 | 1.1754 | 0.8242 | 1 | 1 |
| WCM162_Z1_1 | 12 | 43770087 | 44605113 | 0.2206 | 0.6386 | NA | NA | NA | NA |
| WCM162_Z1_1 | 12 | 44693517 | 56079048 | −0.3146 | 0.0089 | 1.0405 | 0.9719 | 1 | 1 |
| WCM162_Z1_1 | 12 | 56080039 | 56151297 | −0.1114 | 0.2529 | NA | NA | NA | NA |
| WCM162_Z1_1 | 12 | 56182862 | 57132299 | 0.2858 | 0.7127 | 1.7764 | 1.5014 | 2 | 2 |
| WCM162_Z1_1 | 12 | 57133140 | 58240165 | 0.6561 | 1.126 | 2.3579 | 2.0072 | 2 | 2 |
| WCM162_Z1_1 | 12 | 58335505 | 60173312 | −0.2802 | 0.0508 | NA | NA | NA | NA |
| WCM162_Z1_1 | 12 | 62104159 | 63359409 | 0.4925 | 0.9449 | NA | NA | NA | NA |
| WCM162_Z1_1 | 12 | 63541330 | 65762800 | −0.2726 | 0.0599 | NA | NA | NA | NA |
| WCM162_Z1_1 | 12 | 65847540 | 68726027 | 0.4018 | 0.8435 | 2.6322 | 0.9566 | 3 | 1 |
| WCM162_Z1_1 | 12 | 69021118 | 70249147 | 0.9797 | 1.4786 | NA | NA | NA | NA |
| WCM162_Z1_1 | 12 | 70272979 | 71314166 | 0.5648 | 1.0253 | NA | NA | NA | NA |
| WCM162_Z1_1 | 12 | 71441459 | 94727342 | −0.1291 | 0.2319 | 1.4727 | 0.8761 | 1 | 1 |
| WCM162_Z1_1 | 12 | 94729371 | 97019519 | 0.6392 | 1.1075 | 3.3726 | 0.9367 | 3 | 1 |
| WCM162_Z1_1 | 12 | 97020673 | 105239727 | −0.2835 | 0.0467 | 1.093 | 0.9728 | 1 | 1 |
| WCM162_Z1_1 | 12 | 105255146 | 106770201 | 0.5478 | 1.0065 | NA | NA | NA | NA |
| WCM162_Z1_1 | 12 | 106772118 | 108917393 | 0.1184 | 0.5214 | NA | NA | NA | NA |
| WCM162_Z1_1 | 12 | 108918098 | 110405308 | 0.7215 | 1.1979 | 3.6815 | 0.9065 | 4 | 1 |
| WCM162_Z1_1 | 12 | 110418806 | 115120993 | −0.2952 | 0.0325 | 1.0402 | 1.0054 | 1 | 1 |
| WCM162_Z1_1 | 12 | 115801897 | 121018950 | 0.4752 | 0.9256 | 1.9864 | 1.8126 | 2 | 2 |
| WCM162_Z1_1 | 12 | 121079121 | 123247467 | −0.3196 | 0.0028 | 1.0533 | 0.9507 | 1 | 1 |
| WCM162_Z1_1 | 12 | 123249924 | 124497473 | 0.8417 | 1.3291 | 4.0177 | 1.0071 | 4 | 1 |
| WCM162_Z1_1 | 12 | 124796472 | 133810855 | −0.2223 | 0.1205 | 1.1263 | 1.0479 | 1 | 1 |
| WCM162_Z1_1 | 13 | 19600364 | 20077375 | 0.1434 | 0.5501 | NA | NA | NA | NA |
| WCM162_Z1_1 | 13 | 20208095 | 31835189 | 0.4214 | 0.8655 | 2.4487 | 1.1952 | 2 | 1 |
| WCM162_Z1_1 | 13 | 31843397 | 37015300 | 0.0086 | 0.3939 | 1.6974 | 0.9306 | 2 | 1 |
| WCM162_Z1_1 | 13 | 37016366 | 83597934 | −0.7341 | −0.5209 | 1.004 | 0.3899 | 1 | 0 |
| WCM162_Z1_1 | 13 | 84453741 | 112324768 | −0.4977 | −0.2174 | 1.1019 | 0.6183 | 1 | 1 |
| WCM162_Z1_1 | 13 | 112721971 | 115089411 | −0.1596 | 0.1957 | 1.2517 | 1.0388 | 1 | 1 |
| WCM162_Z1_1 | 14 | 19377698 | 22938326 | −0.3045 | 0.0213 | 1.0568 | 0.9729 | 1 | 1 |
| WCM162_Z1_1 | 14 | 22938596 | 25101196 | −0.0985 | 0.2682 | 1.2541 | 1.1545 | 1 | 1 |
| WCM162_Z1_1 | 14 | 25101548 | 35592765 | −0.2757 | 0.0562 | 1.0913 | 0.9881 | 1 | 1 |
| WCM162_Z1_1 | 14 | 35593030 | 38281564 | 0.2297 | 0.6489 | 1.9577 | 1.1783 | 2 | 1 |
| WCM162_Z1_1 | 14 | 38286843 | 54878241 | −0.2448 | 0.0935 | 1.1223 | 1.0116 | 1 | 1 |
| WCM162_Z1_1 | 14 | 54878523 | 60474840 | 0.1886 | 0.602 | 1.9903 | 1.0452 | 2 | 1 |
| WCM162_Z1_1 | 14 | 60483383 | 69619622 | −0.2653 | 0.0687 | 1.1021 | 0.9955 | 1 | 1 |
| WCM162_Z1_1 | 14 | 69676460 | 72176353 | 0.2925 | 0.7202 | 1.7221 | 1.5728 | 2 | 2 |
| WCM162_Z1_1 | 14 | 72190399 | 75989077 | −0.3135 | 0.0103 | 1.0391 | 0.9753 | 1 | 1 |
| WCM162_Z1_1 | 14 | 75991447 | 78374178 | 0.3474 | 0.7823 | 2.2009 | 1.2389 | 2 | 1 |
| WCM162_Z1_1 | 14 | 78390827 | 91467486 | −0.0984 | 0.2683 | 1.2613 | 1.1474 | 1 | 1 |
| WCM162_Z1_1 | 14 | 91592832 | 92905707 | 0.3877 | 0.8277 | 2.6584 | 0.8914 | 3 | 1 |
| WCM162_Z1_1 | 14 | 92908434 | 93998910 | 0.0444 | 0.4357 | NA | NA | NA | NA |
| WCM162_Z1_1 | 14 | 94004472 | 94756690 | −0.3146 | 0.0089 | NA | NA | NA | NA |
| WCM162_Z1_1 | 14 | 94756794 | 98444444 | 0.1549 | 0.5634 | 1.9614 | 0.994 | 2 | 1 |
| WCM162_Z1_1 | 14 | 99182575 | 100625907 | 0.8347 | 1.3214 | NA | NA | NA | NA |
| WCM162_Z1_1 | 14 | 100705782 | 102843174 | 0.2484 | 0.6702 | 2.015 | 1.1675 | 2 | 1 |
| WCM162_Z1_1 | 14 | 102873682 | 104710651 | 0.7451 | 1.2238 | 2.471 | 2.2002 | 2 | 2 |
| WCM162_Z1_1 | 14 | 104979232 | 106354418 | 0.2996 | 0.7283 | 2.3096 | 1.0038 | 2 | 1 |
| WCM162_Z1_1 | 14 | 106357053 | 107283183 | −0.331 | −0.0111 | NA | NA | NA | NA |
| WCM162_Z1_1 | 15 | 20170091 | 24924482 | −0.3211 | 0.0011 | 1.4242 | 0.5773 | 1 | 1 |
| WCM162_Z1_1 | 15 | 25200208 | 31197002 | −0.016 | 0.3652 | 1.3876 | 1.1886 | 1 | 1 |
| WCM162_Z1_1 | 15 | 31197491 | 34782582 | −0.3167 | 0.0064 | 1.0277 | 0.9812 | 1 | 1 |
| WCM162_Z1_1 | 15 | 34812832 | 40556114 | −0.0479 | 0.3278 | 1.4969 | 1.0132 | 1 | 1 |
| WCM162_Z1_1 | 15 | 40557098 | 40764390 | 0.6189 | 1.0851 | NA | NA | NA | NA |
| WCM162_Z1_1 | 15 | 40820970 | 51204336 | −0.278 | 0.0534 | 1.0565 | 1.0188 | 1 | 1 |
| WCM162_Z1_1 | 15 | 51216153 | 54557613 | 0.3521 | 0.7876 | NA | NA | NA | NA |
| WCM162_Z1_1 | 15 | 54586167 | 59430546 | −0.2885 | 0.0406 | 1.0685 | 0.9886 | 1 | 1 |
| WCM162_Z1_1 | 15 | 59445869 | 62146674 | 0.1432 | 0.5498 | NA | NA | NA | NA |
| WCM162_Z1_1 | 15 | 62147082 | 68126118 | −0.2589 | 0.0765 | 1.1156 | 0.9933 | 1 | 1 |
| WCM162_Z1_1 | 15 | 68346664 | 72226021 | 0.0477 | 0.4395 | 1.4507 | 1.2616 | 1 | 1 |
| WCM162_Z1_1 | 15 | 72227724 | 73635840 | 0.4612 | 0.9101 | NA | NA | NA | NA |
| WCM162_Z1_1 | 15 | 73636038 | 74315444 | 0.0607 | 0.4546 | NA | NA | NA | NA |
| WCM162_Z1_1 | 15 | 74315733 | 75722690 | −0.312 | 0.0122 | NA | NA | NA | NA |
| WCM162_Z1_1 | 15 | 75761206 | 78358391 | 0.3159 | 0.7468 | 2.4143 | 0.9418 | 2 | 1 |
| WCM162_Z1_1 | 15 | 78369815 | 81289814 | −0.6019 | −0.3494 | 1.0139 | 0.5559 | 1 | 1 |
| WCM162_Z1_1 | 15 | 81294795 | 85399751 | 0.1269 | 0.5311 | 2.0043 | 0.8857 | 2 | 1 |
| WCM162_Z1_1 | 15 | 85399874 | 89876384 | −0.2798 | 0.0512 | 1.0863 | 0.9859 | 1 | 1 |
| WCM162_Z1_1 | 15 | 89876725 | 90984776 | 0.1715 | 0.5823 | 1.9436 | 1.0509 | 2 | 1 |
| WCM162_Z1_1 | 15 | 90986676 | 102517666 | −0.2631 | 0.0714 | 1.1041 | 0.9974 | 1 | 1 |
| WCM162_Z1_1 | 16 | 66953 | 111914 | −0.3445 | −0.0276 | NA | NA | NA | NA |
| WCM162_Z1_1 | 16 | 112689 | 3778115 | −0.3105 | 0.014 | 1.0245 | 0.995 | 1 | 1 |
| WCM162_Z1_1 | 16 | 3778320 | 5517243 | 0.5075 | 0.9617 | 2.8816 | 1.0136 | 3 | 1 |
| WCM162_Z1_1 | 16 | 5649083 | 10912024 | −0.2403 | 0.099 | NA | NA | NA | NA |
| WCM162_Z1_1 | 16 | 10960357 | 12996399 | 0.5482 | 1.0068 | 2.9612 | 1.0577 | 3 | 1 |
| WCM162_Z1_1 | 16 | 13002434 | 18441854 | −0.3225 | −8.00E−04 | 1.0434 | 0.9556 | 1 | 1 |
| WCM162_Z1_1 | 16 | 18539351 | 18908256 | 0.2857 | 0.7125 | NA | NA | NA | NA |
| WCM162_Z1_1 | 16 | 19020719 | 20566621 | 1.1573 | 1.6692 | NA | NA | NA | NA |
| WCM162_Z1_1 | 16 | 20634806 | 21136559 | 0.1544 | 0.5627 | NA | NA | NA | NA |
| WCM162_Z1_1 | 16 | 21139130 | 22826387 | −0.3262 | −0.0052 | 1.0366 | 0.9563 | 1 | 1 |
| WCM162_Z1_1 | 16 | 22926511 | 24166130 | 0.1626 | 0.5722 | NA | NA | NA | NA |
| WCM162_Z1_1 | 16 | 24183647 | 27802781 | −0.2996 | 0.0272 | NA | NA | NA | NA |
| WCM162_Z1_1 | 16 | 27818833 | 29820906 | 0.5157 | 0.9708 | 3.0082 | 0.9116 | 3 | 1 |
| WCM162_Z1_1 | 16 | 29821596 | 30676211 | 0.0349 | 0.4246 | 1.5952 | 1.0892 | 2 | 1 |
| WCM162_Z1_1 | 16 | 30676370 | 32486609 | 0.5169 | 0.9721 | 2.1362 | 1.7872 | 2 | 2 |
| WCM162_Z1_1 | 16 | 32487093 | 65345363 | −0.6717 | −0.4394 | 1.0733 | 0.4015 | 1 | 0 |
| WCM162_Z1_1 | 16 | 65388168 | 67469632 | 0.0913 | 0.49 | NA | NA | NA | NA |
| WCM162_Z1_1 | 16 | 67469743 | 70891771 | −0.6251 | −0.3792 | 1.0189 | 0.5188 | 1 | 1 |
| WCM162_Z1_1 | 16 | 70894091 | 82753020 | −1.0308 | −0.9253 | 1.0481 | 0.0051 | 1 | 0 |
| WCM162_Z1_1 | 16 | 82882290 | 88502998 | −0.5918 | −0.3365 | 1.5667 | 0.0172 | 2 | 0 |
| WCM162_Z1_1 | 16 | 88503329 | 89846290 | −0.3089 | 0.0159 | 1.9944 | 0.0278 | 2 | 0 |
| WCM162_Z1_1 | 16 | 89849480 | 90162178 | −0.766 | −0.5629 | 1.2094 | 0.1444 | 1 | 0 |
| WCM162_Z1_1 | 17 | 131595 | 1972084 | 0.103 | 0.5035 | 1.8695 | 0.9658 | 2 | 1 |
| WCM162_Z1_1 | 17 | 1985124 | 3819002 | −0.2341 | 0.1064 | 1.1763 | 0.9768 | 1 | 1 |
| WCM162_Z1_1 | 17 | 3819168 | 4356375 | 0.0567 | 0.4499 | NA | NA | NA | NA |
| WCM162_Z1_1 | 17 | 4381887 | 6014100 | 0.5087 | 0.963 | 2.0275 | 1.8713 | 2 | 2 |
| WCM162_Z1_1 | 17 | 6021386 | 7462969 | 0.208 | 0.6241 | 2.1382 | 0.9444 | 2 | 1 |
| WCM162_Z1_1 | 17 | 7463436 | 7942885 | −0.347 | −0.0307 | NA | NA | NA | NA |
| WCM162_Z1_1 | 17 | 7943298 | 9460842 | 0.1928 | 0.6067 | 1.9897 | 1.0559 | 2 | 1 |
| WCM162_Z1_1 | 17 | 9471787 | 11696868 | 0.593 | 1.0565 | 2.1672 | 1.9924 | 2 | 2 |
| WCM162_Z1_1 | 17 | 11700955 | 13504385 | 0.1643 | 0.5741 | NA | NA | NA | NA |
| WCM162_Z1_1 | 17 | 13972942 | 16734869 | −0.3144 | 0.0091 | NA | NA | NA | NA |
| WCM162_Z1_1 | 17 | 16735618 | 19470414 | 0.2002 | 0.6152 | 2.0098 | 1.0537 | 2 | 1 |
| WCM162_Z1_1 | 17 | 19474794 | 20483901 | 0.7011 | 1.1756 | NA | NA | NA | NA |
| WCM162_Z1_1 | 17 | 20769980 | 22023445 | 0.182 | 0.5944 | 1.9318 | 1.0879 | 2 | 1 |
| WCM162_Z1_1 | 17 | 25620970 | 26857856 | −0.2826 | 0.0479 | NA | NA | NA | NA |
| WCM162_Z1_1 | 17 | 26861499 | 29151658 | 0.2917 | 0.7194 | 2.863 | 0.4298 | 3 | 0 |
| WCM162_Z1_1 | 17 | 29159404 | 30806364 | −0.4765 | −0.1909 | 1.8073 | −0.0552 | 2 | 0 |
| WCM162_Z1_1 | 17 | 30806881 | 37459872 | −1.0681 | −0.9784 | 1.0012 | 0.0139 | 1 | 0 |
| WCM162_Z1_1 | 17 | 37499447 | 39114868 | −0.3166 | 0.0066 | 2.0364 | −0.0273 | 2 | 0 |
| WCM162_Z1_1 | 17 | 39240560 | 41833047 | −1.014 | −0.9015 | 1.0229 | 0.0477 | 1 | 0 |
| WCM162_Z1_1 | 17 | 41836085 | 44109474 | −0.2645 | 0.0698 | 2.08 | 0.0191 | 2 | 0 |
| WCM162_Z1_1 | 17 | 44110547 | 45664643 | 0.3044 | 0.7337 | NA | NA | NA | NA |
| WCM162_Z1_1 | 17 | 45677059 | 48072328 | 0.8424 | 1.3298 | 3.8721 | 1.1553 | 4 | 1 |
| WCM162_Z1_1 | 17 | 48133856 | 49062302 | 0.4829 | 0.9343 | 2.8484 | 0.9736 | 3 | 1 |
| WCM162_Z1_1 | 17 | 49063084 | 54588471 | 0.1743 | 0.5856 | NA | NA | NA | NA |
| WCM162_Z1_1 | 17 | 54671811 | 57125157 | −0.3102 | 0.0143 | 1.0591 | 0.9608 | 1 | 1 |
| WCM162_Z1_1 | 17 | 57126560 | 57643129 | 0.5815 | 1.0437 | NA | NA | NA | NA |
| WCM162_Z1_1 | 17 | 57643990 | 59479161 | 0.9473 | 1.4435 | NA | NA | NA | NA |
| WCM162_Z1_1 | 17 | 59480428 | 60140618 | 0.5302 | 0.9869 | NA | NA | NA | NA |
| WCM162_Z1_1 | 17 | 60344524 | 71746508 | −0.2884 | 0.0408 | 1.0723 | 0.985 | 1 | 1 |
| WCM162_Z1_1 | 17 | 71746796 | 72763086 | 0.3196 | 0.7509 | 1.8674 | 1.4983 | 2 | 1 |
| WCM162_Z1_1 | 17 | 72764292 | 73502730 | 1.4192 | 1.9476 | NA | NA | NA | NA |
| WCM162_Z1_1 | 17 | 73503170 | 74533512 | 0.2864 | 0.7133 | 1.9371 | 1.3421 | 2 | 1 |
| WCM162_Z1_1 | 17 | 74534583 | 76045484 | −0.3116 | 0.0125 | NA | NA | NA | NA |
| WCM162_Z1_1 | 17 | 76045691 | 78039924 | 0.1683 | 0.5787 | 1.9851 | 1.0018 | 2 | 1 |
| WCM162_Z1_1 | 17 | 78041446 | 80121181 | 0.5311 | 0.9879 | 2.6023 | 1.3643 | 3 | 1 |
| WCM162_Z1_1 | 17 | 80129716 | 81083455 | −0.1698 | 0.1835 | NA | NA | NA | NA |
| WCM162_Z1_1 | 18 | 48272 | 11868541 | 0.0822 | 0.4795 | 1.7977 | 0.9908 | 2 | 1 |
| WCM162_Z1_1 | 18 | 11872280 | 15005663 | −0.276 | 0.0559 | NA | NA | NA | NA |
| WCM162_Z1_1 | 18 | 15006027 | 19994791 | 0.3311 | 0.7639 | NA | NA | NA | NA |
| WCM162_Z1_1 | 18 | 19995753 | 58310796 | −0.2857 | 0.0441 | 1.0511 | 1.011 | 1 | 1 |
| WCM162_Z1_1 | 18 | 59157840 | 65181659 | 0.2709 | 0.6958 | 2.2158 | 1.0239 | 2 | 1 |
| WCM162_Z1_1 | 18 | 66344342 | 78005205 | −0.2614 | 0.0735 | 1.1069 | 0.9976 | 1 | 1 |
| WCM162_Z1_1 | 19 | 281395 | 375957 | −0.4262 | −0.1282 | NA | NA | NA | NA |
| WCM162_Z1_1 | 19 | 407260 | 619583 | 0.5488 | 1.0075 | NA | NA | NA | NA |
| WCM162_Z1_1 | 19 | 620073 | 1085910 | 0.91 | 1.4032 | 3.4755 | 1.8142 | 3 | 2 |
| WCM162_Z1_1 | 19 | 1085959 | 2280919 | 0.5475 | 1.006 | 2.0084 | 2.0084 | 2 | 2 |
| WCM162_Z1_1 | 19 | 2290147 | 3282486 | 0.1749 | 0.5863 | 1.9712 | 1.0316 | 2 | 1 |
| WCM162_Z1_1 | 19 | 3284943 | 8520452 | −0.2756 | 0.0563 | 1.0637 | 1.0159 | 1 | 1 |
| WCM162_Z1_1 | 19 | 8527426 | 11200257 | 0.0218 | 0.4094 | 1.6252 | 1.031 | 2 | 1 |
| WCM162_Z1_1 | 19 | 11201363 | 13249139 | −0.223 | 0.1198 | 1.1963 | 0.9769 | 1 | 1 |
| WCM162_Z1_1 | 19 | 13249222 | 13616825 | −0.0255 | 0.3541 | NA | NA | NA | NA |
| WCM162_Z1_1 | 19 | 13862624 | 15852274 | 0.1982 | 0.6129 | 1.9613 | 1.0974 | 2 | 1 |
| WCM162_Z1_1 | 19 | 15852700 | 17581460 | −0.0205 | 0.36 | 1.5134 | 1.0535 | 2 | 1 |
| WCM162_Z1_1 | 19 | 17597465 | 19681514 | −0.3342 | −0.015 | 1.0163 | 0.963 | 1 | 1 |
| WCM162_Z1_1 | 19 | 19729371 | 20045557 | 0.2134 | 0.6303 | NA | NA | NA | NA |
| WCM162_Z1_1 | 19 | 20117046 | 33882375 | −0.2918 | 0.0367 | 1.1204 | 0.9311 | 1 | 1 |
| WCM162_Z1_1 | 19 | 33892633 | 35435069 | 0.2756 | 0.7011 | 2.0722 | 1.1792 | 2 | 1 |
| WCM162_Z1_1 | 19 | 35435411 | 42937971 | −0.3171 | 0.0059 | 1.0212 | 0.987 | 1 | 1 |
| WCM162_Z1_1 | 19 | 42946932 | 45404313 | 0.1574 | 0.5662 | 1.9787 | 0.9825 | 2 | 1 |
| WCM162_Z1_1 | 19 | 45404557 | 56321295 | −0.3439 | −0.0268 | 1.0056 | 0.9576 | 1 | 1 |
| WCM162_Z1_1 | 19 | 56321475 | 58565377 | 0.1592 | 0.5682 | 1.9423 | 1.0231 | 2 | 1 |
| WCM162_Z1_1 | 19 | 58570636 | 59083970 | −0.2656 | 0.0684 | 1.1119 | 0.9851 | 1 | 1 |
| WCM162_Z1_1 | 2 | 41623 | 1947007 | −0.0151 | 0.3663 | NA | NA | NA | NA |
| WCM162_Z1_1 | 2 | 1982995 | 9419497 | 0.1451 | 0.552 | 1.9145 | 1.0178 | 2 | 1 |
| WCM162_Z1_1 | 2 | 9437476 | 11053865 | 0.572 | 1.0332 | 2.2052 | 1.888 | 2 | 2 |
| WCM162_Z1_1 | 2 | 11053945 | 11716637 | 0.2521 | 0.6744 | NA | NA | NA | NA |
| WCM162_Z1_1 | 2 | 11718535 | 25326630 | −0.2285 | 0.1131 | 1.1258 | 1.0373 | 1 | 1 |
| WCM162_Z1_1 | 2 | 25344644 | 26846282 | 0.1461 | 0.5532 | 1.9414 | 0.9934 | 2 | 1 |
| WCM162_Z1_1 | 2 | 26852310 | 29449819 | −0.2924 | 0.0359 | 1.06 | 0.9903 | 1 | 1 |
| WCM162_Z1_1 | 2 | 29450510 | 32903986 | 0.2348 | 0.6548 | 2.1484 | 1.0003 | 2 | 1 |
| WCM162_Z1_1 | 2 | 32927963 | 51255400 | −0.2956 | 0.032 | 1.0522 | 0.9926 | 1 | 1 |
| WCM162_Z1_1 | 2 | 52929680 | 65659100 | 0.4378 | 0.8839 | 2.3243 | 1.3663 | 2 | 1 |
| WCM162_Z1_1 | 2 | 66585456 | 69771640 | −0.3528 | −0.0378 | 1.0685 | 0.8799 | 1 | 1 |
| WCM162_Z1_1 | 2 | 69784031 | 72786626 | −0.0359 | 0.3419 | NA | NA | NA | NA |
| WCM162_Z1_1 | 2 | 72802713 | 86712133 | −0.3085 | 0.0164 | 1.0299 | 0.993 | 1 | 1 |
| WCM162_Z1_1 | 2 | 86712969 | 98916552 | −0.0479 | 0.3278 | 1.5338 | 0.9764 | 2 | 1 |
| WCM162_Z1_1 | 2 | 98920194 | 105472888 | −0.2752 | 0.0568 | 1.1321 | 0.9482 | 1 | 1 |
| WCM162_Z1_1 | 2 | 105473048 | 112989489 | 0.2657 | 0.6898 | 2.2014 | 1.0248 | 2 | 1 |
| WCM162_Z1_1 | 2 | 112990918 | 128631706 | −0.3119 | 0.0122 | 1.0428 | 0.9741 | 1 | 1 |
| WCM162_Z1_1 | 2 | 128699703 | 131904331 | 0.4653 | 0.9146 | NA | NA | NA | NA |
| WCM162_Z1_1 | 2 | 132110593 | 138208584 | −0.334 | −0.0148 | 1.1761 | 0.8036 | 1 | 1 |
| WCM162_Z1_1 | 2 | 138320856 | 144525593 | 0.6049 | 1.0696 | NA | NA | NA | NA |
| WCM162_Z1_1 | 2 | 144704689 | 153032137 | 0.0886 | 0.4869 | NA | NA | NA | NA |
| WCM162_Z1_1 | 2 | 153192232 | 160053169 | −0.3004 | 0.0262 | NA | NA | NA | NA |
| WCM162_Z1_1 | 2 | 160055512 | 163693049 | 0.1702 | 0.5809 | 2.0532 | 0.9383 | 2 | 1 |
| WCM162_Z1_1 | 2 | 163695026 | 169439498 | −0.2982 | 0.0289 | 1.1615 | 0.8789 | 1 | 1 |
| WCM162_Z1_1 | 2 | 169551479 | 171191900 | 0.1532 | 0.5614 | 1.952 | 0.9993 | 2 | 1 |
| WCM162_Z1_1 | 2 | 171225777 | 172600665 | −0.3036 | 0.0224 | NA | NA | NA | NA |
| WCM162_Z1_1 | 2 | 172602363 | 177036268 | 0.1953 | 0.6096 | NA | NA | NA | NA |
| WCM162_Z1_1 | 2 | 177036754 | 185803255 | −0.2889 | 0.0402 | 1.0619 | 0.9946 | 1 | 1 |
| WCM162_Z1_1 | 2 | 185803364 | 193059154 | 0.2926 | 0.7204 | 1.9893 | 1.3059 | 2 | 1 |
| WCM162_Z1_1 | 2 | 196544809 | 202900999 | −0.2715 | 0.0613 | 1.0889 | 0.9979 | 1 | 1 |
| WCM162_Z1_1 | 2 | 202938424 | 207176130 | 0.2074 | 0.6235 | 1.9595 | 1.1217 | 2 | 1 |
| WCM162_Z1_1 | 2 | 207310075 | 216300469 | −0.2635 | 0.0709 | 1.0605 | 1.0402 | 1 | 1 |
| WCM162_Z1_1 | 2 | 216808165 | 219554646 | 0.52 | 0.9755 | 3.1468 | 0.786 | 3 | 1 |
| WCM162_Z1_1 | 2 | 219555262 | 219942899 | 0.143 | 0.5497 | NA | NA | NA | NA |
| WCM162_Z1_1 | 2 | 219960006 | 224463141 | −0.2892 | 0.0398 | 1.0878 | 0.9681 | 1 | 1 |
| WCM162_Z1_1 | 2 | 224463170 | 227729815 | 0.2156 | 0.6329 | NA | NA | NA | NA |
| WCM162_Z1_1 | 2 | 227731974 | 231775627 | −0.2767 | 0.055 | 1.0753 | 1.0023 | 1 | 1 |
| WCM162_Z1_1 | 2 | 231861173 | 235405221 | 0.1069 | 0.508 | 1.8801 | 0.9641 | 2 | 1 |
| WCM162_Z1_1 | 2 | 235943655 | 236403422 | 0.6049 | 1.0696 | NA | NA | NA | NA |
| WCM162_Z1_1 | 2 | 236578807 | 242946597 | −0.3319 | −0.0122 | 1.011 | 0.9722 | 1 | 1 |
| WCM162_Z1_1 | 20 | 68351 | 6759629 | −0.3099 | 0.0147 | 1.0373 | 0.9832 | 1 | 1 |
| WCM162_Z1_1 | 20 | 6759706 | 14308144 | 0.5524 | 1.0115 | 3.0868 | 0.9452 | 3 | 1 |
| WCM162_Z1_1 | 20 | 14665558 | 46318901 | −0.3137 | 0.0101 | 1.0438 | 0.9702 | 1 | 1 |
| WCM162_Z1_1 | 20 | 46331321 | 53266980 | 0.2209 | 0.6388 | 2.1078 | 1.0063 | 2 | 1 |
| WCM162_Z1_1 | 20 | 54573624 | 55748206 | −0.4216 | −0.1225 | 0.9652 | 0.8719 | 1 | 1 |
| WCM162_Z1_1 | 20 | 55750050 | 55966818 | 0.2472 | 0.6688 | NA | NA | NA | NA |
| WCM162_Z1_1 | 20 | 55967778 | 57617770 | 0.6704 | 1.1418 | 2.3158 | 2.0974 | 2 | 2 |
| WCM162_Z1_1 | 20 | 57766230 | 60791797 | 0.2469 | 0.6685 | NA | NA | NA | NA |
| WCM162_Z1_1 | 20 | 60791866 | 62904863 | −0.3041 | 0.0217 | 1.0485 | 0.9818 | 1 | 1 |
| WCM162_Z1_1 | 21 | 9826205 | 17250200 | −0.3243 | −0.0029 | 1.0832 | 0.9127 | 1 | 1 |
| WCM162_Z1_1 | 21 | 17250737 | 30464055 | 0.0521 | 0.4446 | 1.7355 | 0.9864 | 2 | 1 |
| WCM162_Z1_1 | 21 | 30464786 | 36079575 | −0.3262 | −0.0052 | 1.0426 | 0.9502 | 1 | 1 |
| WCM162_Z1_1 | 21 | 36080366 | 38132035 | 0.1622 | 0.5717 | 2.0662 | 0.9063 | 2 | 1 |
| WCM162_Z1_1 | 21 | 38137470 | 45064211 | −0.2799 | 0.051 | 1.0561 | 1.0159 | 1 | 1 |
| WCM162_Z1_1 | 21 | 45076528 | 47320528 | 0.1827 | 0.5951 | NA | NA | NA | NA |
| WCM162_Z1_1 | 21 | 47320949 | 48084281 | −0.32 | 0.0023 | 1.0553 | 0.9479 | 1 | 1 |
| WCM162_Z1_1 | 22 | 16287495 | 17671149 | 0.2402 | 0.6609 | NA | NA | NA | NA |
| WCM162_Z1_1 | 22 | 17672662 | 21414708 | 0.5067 | 0.9607 | 2.8061 | 1.0864 | 3 | 1 |
| WCM162_Z1_1 | 22 | 21576156 | 24237463 | 0.1839 | 0.5966 | 2.0437 | 0.9805 | 2 | 1 |
| WCM162_Z1_1 | 22 | 24237682 | 40859227 | −0.2959 | 0.0317 | 1.0458 | 0.9986 | 1 | 1 |
| WCM162_Z1_1 | 22 | 41075635 | 41625569 | 0.1474 | 0.5547 | NA | NA | NA | NA |
| WCM162_Z1_1 | 22 | 41626190 | 43089470 | 0.5277 | 0.9841 | 2.0968 | 1.8595 | 2 | 2 |
| WCM162_Z1_1 | 22 | 43089849 | 44028074 | 0.1457 | 0.5528 | 1.9357 | 0.9981 | 2 | 1 |
| WCM162_Z1_1 | 22 | 44031058 | 46449730 | −0.298 | 0.0291 | 1.0477 | 0.9931 | 1 | 1 |
| WCM162_Z1_1 | 22 | 46449893 | 48940506 | 0.1834 | 0.596 | 1.9581 | 1.0649 | 2 | 1 |
| WCM162_Z1_1 | 22 | 48942353 | 51215137 | −0.2861 | 0.0435 | 1.0619 | 0.9994 | 1 | 1 |
| WCM162_Z1_1 | 3 | 239347 | 3888402 | −0.364 | −0.0515 | NA | NA | NA | NA |
| WCM162_Z1_1 | 3 | 4110270 | 7649764 | 0.2887 | 0.716 | NA | NA | NA | NA |
| WCM162_Z1_1 | 3 | 7721939 | 9945733 | −0.3554 | −0.0409 | 1.0018 | 0.9422 | 1 | 1 |
| WCM162_Z1_1 | 3 | 9948050 | 11350521 | 0.166 | 0.5761 | 1.9731 | 1.0085 | 2 | 1 |
| WCM162_Z1_1 | 3 | 11354843 | 13399786 | 0.5632 | 1.0235 | 3.0058 | 1.0598 | 3 | 1 |
| WCM162_Z1_1 | 3 | 13401875 | 13671430 | 0.2249 | 0.6434 | NA | NA | NA | NA |
| WCM162_Z1_1 | 3 | 13672256 | 15269462 | −0.3085 | 0.0164 | NA | NA | NA | NA |
| WCM162_Z1_1 | 3 | 15270566 | 18390752 | 0.2117 | 0.6284 | NA | NA | NA | NA |
| WCM162_Z1_1 | 3 | 18391160 | 36873819 | −0.2392 | 0.1002 | 1.2205 | 0.9234 | 1 | 1 |
| WCM162_Z1_1 | 3 | 36874135 | 38042425 | 0.1871 | 0.6002 | NA | NA | NA | NA |
| WCM162_Z1_1 | 3 | 38042993 | 40574559 | 0.4503 | 0.8979 | 1.9571 | 1.7696 | 2 | 2 |
| WCM162_Z1_1 | 3 | 41265567 | 41723263 | 0.132 | 0.537 | NA | NA | NA | NA |
| WCM162_Z1_1 | 3 | 41746628 | 47870597 | −0.3084 | 0.0165 | 1.0226 | 1.0004 | 1 | 1 |
| WCM162_Z1_1 | 3 | 47882580 | 47894745 | 0.6337 | 1.1014 | NA | NA | NA | NA |
| WCM162_Z1_1 | 3 | 47896774 | 48573754 | 0.4302 | 0.8754 | NA | NA | NA | NA |
| WCM162_Z1_1 | 3 | 48575930 | 49570882 | 0.725 | 1.2018 | 2.6983 | 1.9021 | 3 | 2 |
| WCM162_Z1_1 | 3 | 49662635 | 49845471 | 0.176 | 0.5875 | NA | NA | NA | NA |
| WCM162_Z1_1 | 3 | 49845842 | 51317578 | −0.2968 | 0.0306 | NA | NA | NA | NA |
| WCM162_Z1_1 | 3 | 51347686 | 53920940 | 0.1716 | 0.5825 | 1.9369 | 1.0579 | 2 | 1 |
| WCM162_Z1_1 | 3 | 53922508 | 63661228 | −0.2687 | 0.0646 | 1.0736 | 1.018 | 1 | 1 |
| WCM162_Z1_1 | 3 | 63805214 | 67275920 | 0.4115 | 0.8545 | NA | NA | NA | NA |
| WCM162_Z1_1 | 3 | 67411103 | 112299732 | −0.2778 | 0.0537 | 1.0668 | 1.009 | 1 | 1 |
| WCM162_Z1_1 | 3 | 112300090 | 119373355 | 0.15 | 0.5576 | 1.9235 | 1.0203 | 2 | 1 |
| WCM162_Z1_1 | 3 | 119378872 | 121563395 | −0.3089 | 0.0158 | NA | NA | NA | NA |
| WCM162_Z1_1 | 3 | 121573628 | 133907702 | 0.1914 | 0.6051 | 2.045 | 0.9972 | 2 | 1 |
| WCM162_Z1_1 | 3 | 133910803 | 143704616 | −0.2864 | 0.0433 | 1.0579 | 1.0029 | 1 | 1 |
| WCM162_Z1_1 | 3 | 143708352 | 148757885 | 0.4423 | 0.8889 | NA | NA | NA | NA |
| WCM162_Z1_1 | 3 | 148759439 | 167351216 | −0.2082 | 0.1375 | 1.1383 | 1.0617 | 1 | 1 |
| WCM162_Z1_1 | 3 | 167371084 | 167543112 | 0.549 | 1.0078 | NA | NA | NA | NA |
| WCM162_Z1_1 | 3 | 167728070 | 169381124 | −0.2983 | 0.0288 | NA | NA | NA | NA |
| WCM162_Z1_1 | 3 | 169482658 | 174577202 | 0.2278 | 0.6468 | NA | NA | NA | NA |
| WCM162_Z1_1 | 3 | 174814784 | 180650188 | −0.2996 | 0.0272 | NA | NA | NA | NA |
| WCM162_Z1_1 | 3 | 180651130 | 183967556 | 0.3137 | 0.7442 | 1.7982 | 1.5519 | 2 | 2 |
| WCM162_Z1_1 | 3 | 183975460 | 189038499 | −0.3226 | −8.00E−04 | 1.0628 | 0.9361 | 1 | 1 |
| WCM162_Z1_1 | 3 | 189349353 | 190374214 | 0.2442 | 0.6654 | NA | NA | NA | NA |
| WCM162_Z1_1 | 3 | 190573229 | 194408362 | −0.3057 | 0.0198 | 1.0977 | 0.9299 | 1 | 1 |
| WCM162_Z1_1 | 3 | 194408704 | 197238772 | 0.2035 | 0.619 | 2.0939 | 0.9776 | 2 | 1 |
| WCM162_Z1_1 | 3 | 197239101 | 197894726 | −0.3555 | −0.041 | NA | NA | NA | NA |
| WCM162_Z1_1 | 4 | 59404 | 3221934 | 0.1996 | 0.6145 | 2.1437 | 0.9183 | 2 | 1 |
| WCM162_Z1_1 | 4 | 3224184 | 15003656 | −0.2973 | 0.03 | 1.0557 | 0.9863 | 1 | 1 |
| WCM162_Z1_1 | 4 | 15003936 | 17666205 | 0.527 | 0.9834 | 2.9875 | 0.9667 | 3 | 1 |
| WCM162_Z1_1 | 4 | 17690076 | 24914486 | −0.1077 | 0.2573 | NA | NA | NA | NA |
| WCM162_Z1_1 | 4 | 25001439 | 27162478 | 0.4224 | 0.8666 | NA | NA | NA | NA |
| WCM162_Z1_1 | 4 | 28364214 | 37828373 | −0.3387 | −0.0204 | NA | NA | NA | NA |
| WCM162_Z1_1 | 4 | 37831658 | 41699194 | 0.2809 | 0.7071 | 2.1585 | 1.1066 | 2 | 1 |
| WCM162_Z1_1 | 4 | 41747853 | 46263926 | −0.3201 | 0.0023 | NA | NA | NA | NA |
| WCM162_Z1_1 | 4 | 46305495 | 48583588 | 0.2117 | 0.6284 | 2.0278 | 1.0639 | 2 | 1 |
| WCM162_Z1_1 | 4 | 48588742 | 56225539 | −0.3261 | −0.0051 | NA | NA | NA | NA |
| WCM162_Z1_1 | 4 | 56230378 | 70361342 | 0.1453 | 0.5523 | 2.0042 | 0.9286 | 2 | 1 |
| WCM162_Z1_1 | 4 | 70361543 | 73149405 | −0.2755 | 0.0564 | NA | NA | NA | NA |
| WCM162_Z1_1 | 4 | 73154498 | 75971423 | 0.5552 | 1.0147 | NA | NA | NA | NA |
| WCM162_Z1_1 | 4 | 76407839 | 105412771 | −0.283 | 0.0473 | 1.0709 | 0.9957 | 1 | 1 |
| WCM162_Z1_1 | 4 | 106155296 | 109461954 | 0.1525 | 0.5605 | NA | NA | NA | NA |
| WCM162_Z1_1 | 4 | 109478108 | 110460786 | −0.6213 | −0.3743 | NA | NA | NA | NA |
| WCM162_Z1_1 | 4 | 110580185 | 113540768 | 0.2688 | 0.6934 | 2.6236 | 0.6107 | 3 | 1 |
| WCM162_Z1_1 | 4 | 113541183 | 113825648 | 0.0058 | 0.3907 | NA | NA | NA | NA |
| WCM162_Z1_1 | 4 | 113970897 | 119256710 | −0.66 | −0.4243 | NA | NA | NA | NA |
| WCM162_Z1_1 | 4 | 119259449 | 143044460 | −0.1713 | 0.1817 | 1.4063 | 0.8621 | 1 | 1 |
| WCM162_Z1_1 | 4 | 143045817 | 149358014 | 0.4273 | 0.8721 | 2.0189 | 1.6417 | 2 | 2 |
| WCM162_Z1_1 | 4 | 151000419 | 165118755 | −0.304 | 0.0219 | 1.0486 | 0.9819 | 1 | 1 |
| WCM162_Z1_1 | 4 | 165878405 | 166795069 | 0.2193 | 0.6371 | NA | NA | NA | NA |
| WCM162_Z1_1 | 4 | 166910635 | 171010772 | 0.8043 | 1.2884 | NA | NA | NA | NA |
| WCM162_Z1_1 | 4 | 172735808 | 175604041 | 0.2319 | 0.6514 | NA | NA | NA | NA |
| WCM162_Z1_1 | 4 | 175636717 | 186997882 | −0.2852 | 0.0447 | 1.0678 | 0.9952 | 1 | 1 |
| WCM162_Z1_1 | 4 | 186998098 | 190426502 | 0.1916 | 0.6054 | NA | NA | NA | NA |
| WCM162_Z1_1 | 5 | 140443 | 796297 | −0.281 | 0.0497 | 1.0702 | 0.9999 | 1 | 1 |
| WCM162_Z1_1 | 5 | 814886 | 32786413 | −0.0347 | 0.3432 | 1.5261 | 1.0112 | 2 | 1 |
| WCM162_Z1_1 | 5 | 32789694 | 36208784 | 0.3328 | 0.7658 | 1.804 | 1.5967 | 2 | 2 |
| WCM162_Z1_1 | 5 | 36211967 | 53751988 | −0.0331 | 0.3452 | 1.5743 | 0.9664 | 2 | 1 |
| WCM162_Z1_1 | 5 | 53814062 | 63894249 | −0.3376 | −0.0191 | 1.0544 | 0.9193 | 1 | 1 |
| WCM162_Z1_1 | 5 | 63905079 | 65474605 | 0.1869 | 0.6 | NA | NA | NA | NA |
| WCM162_Z1_1 | 5 | 65892597 | 96519230 | −0.2708 | 0.0621 | 1.0642 | 1.0237 | 1 | 1 |
| WCM162_Z1_1 | 5 | 98109828 | 108436193 | −1.0375 | −0.9348 | NA | NA | NA | NA |
| WCM162_Z1_1 | 5 | 108516527 | 109106104 | −0.0906 | 0.2775 | NA | NA | NA | NA |
| WCM162_Z1_1 | 5 | 109110537 | 114861304 | 0.7098 | 1.1851 | 2.4408 | 2.1067 | 2 | 2 |
| WCM162_Z1_1 | 5 | 114878759 | 118507544 | −0.3229 | −0.0012 | NA | NA | NA | NA |
| WCM162_Z1_1 | 5 | 118510994 | 124080614 | 0.1802 | 0.5924 | 1.6476 | 1.3678 | 2 | 1 |
| WCM162_Z1_1 | 5 | 125696025 | 135416296 | −0.2556 | 0.0805 | 1.074 | 1.0408 | 1 | 1 |
| WCM162_Z1_1 | 5 | 135469500 | 137904647 | 0.7671 | 1.2478 | NA | NA | NA | NA |
| WCM162_Z1_1 | 5 | 137906805 | 140774636 | −0.3272 | −0.0065 | 1.0271 | 0.9639 | 1 | 1 |
| WCM162_Z1_1 | 5 | 140778259 | 143059433 | 0.1918 | 0.6057 | 2.0371 | 1.0063 | 2 | 1 |
| WCM162_Z1_1 | 5 | 143191860 | 147863825 | −0.1818 | 0.1692 | 1.1957 | 1.0532 | 1 | 1 |
| WCM162_Z1_1 | 5 | 147870374 | 150257594 | 0.144 | 0.5508 | 1.9685 | 0.9612 | 2 | 1 |
| WCM162_Z1_1 | 5 | 150275138 | 156945875 | −0.2524 | 0.0844 | 1.1165 | 1.0039 | 1 | 1 |
| WCM162_Z1_1 | 5 | 156946942 | 173035285 | 0.1228 | 0.5264 | 1.5897 | 1.291 | 2 | 1 |
| WCM162_Z1_1 | 5 | 173036261 | 180687773 | −0.3221 | −2.00E−04 | 1.0291 | 0.9706 | 1 | 1 |
| WCM162_Z1_1 | 6 | 203552 | 22146924 | −0.2956 | 0.0321 | 1.0461 | 0.9989 | 1 | 1 |
| WCM162_Z1_1 | 6 | 22147408 | 25450202 | 0.1573 | 0.5661 | 1.9871 | 0.9739 | 2 | 1 |
| WCM162_Z1_1 | 6 | 25450617 | 33235641 | −0.3229 | −0.0012 | 1.0138 | 0.9846 | 1 | 1 |
| WCM162_Z1_1 | 6 | 33235755 | 35200740 | 0.0166 | 0.4033 | 1.4536 | 1.1914 | 1 | 1 |
| WCM162_Z1_1 | 6 | 35200985 | 40998158 | −0.2904 | 0.0384 | 1.0706 | 0.9833 | 1 | 1 |
| WCM162_Z1_1 | 6 | 40998395 | 41310669 | 0.1572 | 0.5659 | NA | NA | NA | NA |
| WCM162_Z1_1 | 6 | 41318438 | 44272221 | 0.8001 | 1.2838 | 3.1037 | 1.766 | 3 | 2 |
| WCM162_Z1_1 | 6 | 44272503 | 46216493 | 0.4206 | 0.8646 | NA | NA | NA | NA |
| WCM162_Z1_1 | 6 | 46293210 | 62887088 | −0.2873 | 0.0421 | 1.0496 | 1.0096 | 1 | 1 |
| WCM162_Z1_1 | 6 | 62995834 | 65525121 | 0.2466 | 0.6681 | 2.052 | 1.126 | 2 | 1 |
| WCM162_Z1_1 | 6 | 65531555 | 73516585 | −0.2321 | 0.1088 | 1.1198 | 1.0368 | 1 | 1 |
| WCM162_Z1_1 | 6 | 73677432 | 84930814 | 0.2023 | 0.6177 | 2.0097 | 1.0591 | 2 | 1 |
| WCM162_Z1_1 | 6 | 84936092 | 99282761 | −0.2944 | 0.0335 | 1.103 | 0.9439 | 1 | 1 |
| WCM162_Z1_1 | 6 | 99283328 | 101315842 | 0.91 | 1.4032 | NA | NA | NA | NA |
| WCM162_Z1_1 | 6 | 101847171 | 131277564 | −0.2342 | 0.1063 | 1.1037 | 1.0492 | 1 | 1 |
| WCM162_Z1_1 | 6 | 131466515 | 135507079 | 0.1662 | 0.5763 | 1.9152 | 1.0668 | 2 | 1 |
| WCM162_Z1_1 | 6 | 135509024 | 138833430 | 0.8363 | 1.3233 | 2.8616 | 2.143 | 3 | 2 |
| WCM162_Z1_1 | 6 | 138892979 | 149395233 | −0.0586 | 0.3152 | 1.3862 | 1.1022 | 1 | 1 |
| WCM162_Z1_1 | 6 | 149691202 | 152489038 | 0.4733 | 0.9235 | 2.4321 | 1.3613 | 2 | 1 |
| WCM162_Z1_1 | 6 | 152489294 | 153332747 | 0.0293 | 0.4181 | NA | NA | NA | NA |
| WCM162_Z1_1 | 6 | 153344530 | 156275691 | −0.3049 | 0.0208 | NA | NA | NA | NA |
| WCM162_Z1_1 | 6 | 157099205 | 159316276 | 0.2594 | 0.6828 | NA | NA | NA | NA |
| WCM162_Z1_1 | 6 | 159331220 | 170893435 | −0.2972 | 0.0301 | 1.1402 | 0.902 | 1 | 1 |
| WCM162_Z1_1 | 7 | 193265 | 3205705 | −0.2657 | 0.0683 | 1.0865 | 1.0105 | 1 | 1 |
| WCM162_Z1_1 | 7 | 3658806 | 5460136 | 0.2112 | 0.6278 | 2.0093 | 1.0811 | 2 | 1 |
| WCM162_Z1_1 | 7 | 5460239 | 6205440 | −0.2285 | 0.1132 | NA | NA | NA | NA |
| WCM162_Z1_1 | 7 | 6210248 | 8791392 | 0.3356 | 0.769 | 1.7811 | 1.6272 | 2 | 2 |
| WCM162_Z1_1 | 7 | 10973282 | 14249142 | −0.3139 | 0.0098 | 1.1147 | 0.8989 | 1 | 1 |
| WCM162_Z1_1 | 7 | 14378237 | 16921571 | 0.2096 | 0.6259 | NA | NA | NA | NA |
| WCM162_Z1_1 | 7 | 17338900 | 18535851 | −0.3431 | −0.0258 | NA | NA | NA | NA |
| WCM162_Z1_1 | 7 | 18617811 | 28060255 | 0.2403 | 0.6609 | 1.8908 | 1.2714 | 2 | 1 |
| WCM162_Z1_1 | 7 | 28111248 | 31003690 | 0.7583 | 1.2382 | 2.7097 | 2.0083 | 3 | 2 |
| WCM162_Z1_1 | 7 | 31008552 | 42079796 | 0.3387 | 0.7725 | 2.4042 | 1.0123 | 2 | 1 |
| WCM162_Z1_1 | 7 | 42085094 | 44807194 | 0.7169 | 1.1929 | 3.5565 | 1.0158 | 4 | 1 |
| WCM162_Z1_1 | 7 | 44838852 | 56022658 | 0.1769 | 0.5886 | 1.9656 | 1.0419 | 2 | 1 |
| WCM162_Z1_1 | 7 | 56022865 | 65233274 | −0.2737 | 0.0586 | 1.2886 | 0.7943 | 1 | 1 |
| WCM162_Z1_1 | 7 | 65305416 | 72892707 | 0.5846 | 1.0472 | NA | NA | NA | NA |
| WCM162_Z1_1 | 7 | 72966569 | 77045605 | 0.095 | 0.4943 | 1.9654 | 0.8518 | 2 | 1 |
| WCM162_Z1_1 | 7 | 77166958 | 79082370 | 0.7867 | 1.2691 | NA | NA | NA | NA |
| WCM162_Z1_1 | 7 | 79082596 | 92354972 | −0.2593 | 0.076 | 1.2451 | 0.8631 | 1 | 1 |
| WCM162_Z1_1 | 7 | 92404097 | 95053889 | 0.2194 | 0.6371 | NA | NA | NA | NA |
| WCM162_Z1_1 | 7 | 95115426 | 96650360 | −0.1711 | 0.1819 | NA | NA | NA | NA |
| WCM162_Z1_1 | 7 | 96651501 | 100091311 | 0.9506 | 1.4471 | 4.2763 | 1.177 | 4 | 1 |
| WCM162_Z1_1 | 7 | 100137121 | 102881051 | −0.3369 | −0.0183 | 1.1455 | 0.8293 | 1 | 1 |
| WCM162_Z1_1 | 7 | 102895234 | 103290786 | 0.0594 | 0.4531 | NA | NA | NA | NA |
| WCM162_Z1_1 | 7 | 103292176 | 108206300 | −0.2792 | 0.0519 | 1.0894 | 0.9839 | 1 | 1 |
| WCM162_Z1_1 | 7 | 108209988 | 116199331 | 0.125 | 0.5289 | NA | NA | NA | NA |
| WCM162_Z1_1 | 7 | 116339224 | 122091469 | 0.4765 | 0.9271 | NA | NA | NA | NA |
| WCM162_Z1_1 | 7 | 122111493 | 133261429 | −0.0308 | 0.3479 | 1.4756 | 1.0697 | 1 | 1 |
| WCM162_Z1_1 | 7 | 133314861 | 135123067 | 0.7029 | 1.1775 | 2.8822 | 1.6415 | 3 | 2 |
| WCM162_Z1_1 | 7 | 135255995 | 137172383 | −0.0408 | 0.3361 | NA | NA | NA | NA |
| WCM162_Z1_1 | 7 | 137178573 | 139226819 | 0.6229 | 1.0895 | 3.1726 | 1.0833 | 3 | 1 |
| WCM162_Z1_1 | 7 | 139229024 | 142334894 | 0.1031 | 0.5036 | 1.7223 | 1.1132 | 2 | 1 |
| WCM162_Z1_1 | 7 | 142353505 | 144708009 | 0.9873 | 1.4868 | 4.5827 | 1.0226 | 5 | 1 |
| WCM162_Z1_1 | 7 | 145601241 | 150020619 | 0.2032 | 0.6186 | 1.6477 | 1.4231 | 2 | 1 |
| WCM162_Z1_1 | 7 | 150027559 | 152142849 | 0.6283 | 1.0954 | 3.2583 | 1.0152 | 3 | 1 |
| WCM162_Z1_1 | 7 | 152143261 | 158937375 | 0.2809 | 0.7072 | 1.79 | 1.4752 | 2 | 1 |
| WCM162_Z1_1 | 8 | 190897 | 4851916 | −1.049 | −0.9511 | 1.0337 | 8.00E−04 | 1 | 0 |
| WCM162_Z1_1 | 8 | 6266812 | 7215653 | −0.3288 | −0.0084 | NA | NA | NA | NA |
| WCM162_Z1_1 | 8 | 7215836 | 12004001 | −1.0674 | −0.9775 | 1.0236 | −0.0078 | 1 | 0 |
| WCM162_Z1_1 | 8 | 12042922 | 20112642 | −0.323 | −0.0013 | 2.013 | −0.0148 | 2 | 0 |
| WCM162_Z1_1 | 8 | 21550826 | 29953045 | −1.0569 | −0.9624 | 0.9836 | 0.0428 | 1 | 0 |
| WCM162_Z1_1 | 8 | 29953401 | 55576321 | −0.2508 | 0.0863 | 1.1052 | 1.0181 | 1 | 1 |
| WCM162_Z1_1 | 8 | 55586412 | 59872515 | 0.1602 | 0.5695 | NA | NA | NA | NA |
| WCM162_Z1_1 | 8 | 60031528 | 68972954 | −0.3193 | 0.0032 | 1.0401 | 0.9643 | 1 | 1 |
| WCM162_Z1_1 | 8 | 68981359 | 69728143 | 0.2525 | 0.6749 | NA | NA | NA | NA |
| WCM162_Z1_1 | 8 | 69730480 | 72948672 | 0.9511 | 1.4476 | NA | NA | NA | NA |
| WCM162_Z1_1 | 8 | 72950215 | 75171708 | 0.367 | 0.8044 | 2.5348 | 0.9581 | 3 | 1 |
| WCM162_Z1_1 | 8 | 75227143 | 101937169 | −0.2295 | 0.112 | 1.16 | 1.0014 | 1 | 1 |
| WCM162_Z1_1 | 8 | 101960329 | 109009148 | 0.1869 | 0.6 | 1.9964 | 1.0351 | 2 | 1 |
| WCM162_Z1_1 | 8 | 109094770 | 118817117 | 0.6136 | 1.0792 | 3.1694 | 1.0563 | 3 | 1 |
| WCM162_Z1_1 | 8 | 118819560 | 124801970 | 0.2699 | 0.6946 | 2.1845 | 1.0523 | 2 | 1 |
| WCM162_Z1_1 | 8 | 124810427 | 131955674 | −0.23 | 0.1114 | 1.1777 | 0.9828 | 1 | 1 |
| WCM162_Z1_1 | 8 | 131964193 | 144650032 | 0.2147 | 0.6317 | 2.0394 | 1.0594 | 2 | 1 |
| WCM162_Z1_1 | 8 | 144650375 | 145732358 | 0.8379 | 1.325 | 3.7506 | 1.2601 | 4 | 1 |
| WCM162_Z1_1 | 8 | 145734792 | 146076708 | 0.2959 | 0.7242 | 2.2677 | 1.0362 | 2 | 1 |
| WCM162_Z1_1 | 8 | 146105825 | 146279527 | −0.3454 | −0.0287 | NA | NA | NA | NA |
| WCM162_Z1_1 | 9 | 17074 | 710966 | 0.1815 | 0.5939 | 2.0763 | 0.9422 | 2 | 1 |
| WCM162_Z1_1 | 9 | 711396 | 3228871 | 0.4978 | 0.9509 | 2.0751 | 1.791 | 2 | 2 |
| WCM162_Z1_1 | 9 | 3248025 | 8528711 | 0.1979 | 0.6126 | 2.0465 | 1.0114 | 2 | 1 |
| WCM162_Z1_1 | 9 | 8633445 | 15177778 | 0.8652 | 1.3547 | 4.2151 | 0.8996 | 4 | 1 |
| WCM162_Z1_1 | 9 | 15182370 | 18950787 | 0.3486 | 0.7837 | NA | NA | NA | NA |
| WCM162_Z1_1 | 9 | 19032906 | 20789594 | 0.8222 | 1.3079 | NA | NA | NA | NA |
| WCM162_Z1_1 | 9 | 20819849 | 23765050 | 0.2489 | 0.6708 | NA | NA | NA | NA |
| WCM162_Z1_1 | 9 | 24543256 | 26116168 | 1.2384 | 1.7558 | NA | NA | NA | NA |
| WCM162_Z1_1 | 9 | 26842384 | 35562430 | 0.4123 | 0.8553 | 2.4258 | 1.1924 | 2 | 1 |
| WCM162_Z1_1 | 9 | 35562895 | 37955953 | 0.7734 | 1.2547 | 2.579 | 2.1934 | 3 | 2 |
| WCM162_Z1_1 | 9 | 37974743 | 38424245 | 0.5624 | 1.0226 | NA | NA | NA | NA |
| WCM162_Z1_1 | 9 | 38527117 | 71866216 | 0.1232 | 0.5268 | 1.7628 | 1.1187 | 2 | 1 |
| WCM162_Z1_1 | 9 | 71867751 | 75407265 | 0.6589 | 1.1292 | NA | NA | NA | NA |
| WCM162_Z1_1 | 9 | 75420379 | 82006446 | −0.0836 | 0.2858 | 1.278 | 1.1601 | 1 | 1 |
| WCM162_Z1_1 | 9 | 82006548 | 85677674 | 0.3613 | 0.798 | NA | NA | NA | NA |
| WCM162_Z1_1 | 9 | 85863188 | 88457757 | −0.3136 | 0.0101 | NA | NA | NA | NA |
| WCM162_Z1_1 | 9 | 88464286 | 94519715 | 0.3263 | 0.7585 | 2.2842 | 1.0992 | 2 | 1 |
| WCM162_Z1_1 | 9 | 94519838 | 95475744 | 1.1311 | 1.6412 | NA | NA | NA | NA |
| WCM162_Z1_1 | 9 | 95477468 | 97369228 | 0.3514 | 0.7869 | NA | NA | NA | NA |
| WCM162_Z1_1 | 9 | 97372256 | 100105735 | −0.2846 | 0.0453 | NA | NA | NA | NA |
| WCM162_Z1_1 | 9 | 100107898 | 102068867 | 0.4688 | 0.9186 | 2.4578 | 1.3227 | 2 | 1 |
| WCM162_Z1_1 | 9 | 102589053 | 123870111 | −0.2547 | 0.0816 | 1.085 | 1.0314 | 1 | 1 |
| WCM162_Z1_1 | 9 | 123874854 | 130191186 | 0.3557 | 0.7917 | 2.2812 | 1.181 | 2 | 1 |
| WCM162_Z1_1 | 9 | 130197414 | 130825931 | 0.5986 | 1.0626 | NA | NA | NA | NA |
| WCM162_Z1_1 | 9 | 130826167 | 131900037 | 0.2841 | 0.7108 | 2.1991 | 1.0743 | 2 | 1 |
| WCM162_Z1_1 | 9 | 131904750 | 141111414 | −0.2955 | 0.0322 | 1.1058 | 0.9393 | 1 | 1 |
| WCM162_Z1_1 | X | 2700156 | 31341744 | −0.2953 | 0.0324 | NA | NA | NA | NA |
| WCM162_Z1_1 | X | 31366746 | 31986543 | −2.005 | −2.6414 | NA | NA | NA | NA |
| WCM162_Z1_1 | X | 32235149 | 43741497 | −0.2948 | 0.033 | NA | NA | NA | NA |
| WCM162_Z1_1 | X | 43809119 | 46696611 | 0.6992 | 1.1735 | NA | NA | NA | NA |
| WCM162_Z1_1 | X | 46713261 | 49075803 | 1.6762 | 2.218 | NA | NA | NA | NA |
| WCM162_Z1_1 | X | 49079035 | 55650231 | 0.8533 | 1.3417 | NA | NA | NA | NA |
| WCM162_Z1_1 | X | 55650703 | 78622653 | −0.3147 | 0.0088 | NA | NA | NA | NA |
| WCM162_Z1_1 | X | 79277940 | 80552709 | 0.8131 | 1.2979 | NA | NA | NA | NA |
| WCM162_Z1_1 | X | 82763411 | 86071084 | −0.3818 | −0.0733 | NA | NA | NA | NA |
| WCM162_Z1_1 | X | 86773300 | 100613625 | 0.0701 | 0.4654 | NA | NA | NA | NA |
| WCM162_Z1_1 | X | 100614336 | 123234423 | −0.3579 | −0.0439 | NA | NA | NA | NA |
| WCM162_Z1_1 | X | 123480504 | 125955487 | 0.454 | 0.902 | NA | NA | NA | NA |
| WCM162_Z1_1 | X | 127185439 | 132548850 | −0.3572 | −0.0431 | NA | NA | NA | NA |
| WCM162_Z1_1 | X | 132670164 | 134715513 | 0.5139 | 0.9688 | NA | NA | NA | NA |
| WCM162_Z1_1 | X | 134989113 | 154509348 | −0.3094 | 0.0152 | NA | NA | NA | NA |
| WCM162_Z9_1 | 1 | 69594 | 249212550 | 0.0059 | NA | NA | NA | NA | NA |
| WCM162_Z9_1 | 10 | 225960 | 135440080 | 0.0115 | NA | NA | NA | NA | NA |
| WCM162_Z9_1 | 11 | 130546 | 134856523 | −0.0072 | NA | NA | NA | NA | NA |
| WCM162_Z9_1 | 12 | 148674 | 133810712 | 0.0351 | NA | NA | NA | NA | NA |
| WCM162_Z9_1 | 13 | 19600364 | 115089411 | 0.0068 | NA | NA | NA | NA | NA |
| WCM162_Z9_1 | 14 | 19377737 | 107283019 | 0.0137 | NA | NA | NA | NA | NA |
| WCM162_Z9_1 | 15 | 20170133 | 102517678 | −0.0018 | NA | NA | NA | NA | NA |
| WCM162_Z9_1 | 16 | 66953 | 3824584 | −0.0344 | NA | NA | NA | NA | NA |
| WCM162_Z9_1 | 16 | 3828064 | 33961794 | 0.0491 | NA | NA | NA | NA | NA |
| WCM162_Z9_1 | 16 | 33962238 | 90162177 | −0.042 | NA | NA | NA | NA | NA |
| WCM162_Z9_1 | 17 | 131595 | 29585440 | 0.0282 | NA | NA | NA | NA | NA |
| WCM162_Z9_1 | 17 | 29587440 | 41958701 | −0.0912 | NA | NA | NA | NA | NA |
| WCM162_Z9_1 | 17 | 41958941 | 72709000 | 0.0337 | NA | NA | NA | NA | NA |
| WCM162_Z9_1 | 17 | 72725434 | 73494805 | 0.3207 | NA | NA | NA | NA | NA |
| WCM162_Z9_1 | 17 | 73495162 | 81083455 | 0.0014 | NA | NA | NA | NA | NA |
| WCM162_Z9_1 | 18 | 48221 | 78005199 | −0.018 | NA | NA | NA | NA | NA |
| WCM162_Z9_1 | 19 | 281413 | 59083985 | −0.0305 | NA | NA | NA | NA | NA |
| WCM162_Z9_1 | 2 | 41614 | 242946604 | 0.0262 | NA | NA | NA | NA | NA |
| WCM162_Z9_1 | 20 | 68373 | 62904835 | −0.0109 | NA | NA | NA | NA | NA |
| WCM162_Z9_1 | 21 | 9826207 | 48084226 | −0.0156 | NA | NA | NA | NA | NA |
| WCM162_Z9_1 | 22 | 16287497 | 51215123 | −0.0126 | NA | NA | NA | NA | NA |
| WCM162_Z9_1 | 3 | 239347 | 197894720 | 0.0164 | NA | NA | NA | NA | NA |
| WCM162_Z9_1 | 4 | 59408 | 190426502 | 0.0304 | NA | NA | NA | NA | NA |
| WCM162_Z9_1 | 5 | 140663 | 180687803 | 0.0119 | NA | NA | NA | NA | NA |
| WCM162_Z9_1 | 6 | 203552 | 170893408 | 0.0093 | NA | NA | NA | NA | NA |
| WCM162_Z9_1 | 7 | 193253 | 158937400 | 0.0577 | NA | NA | NA | NA | NA |
| WCM162_Z9_1 | 8 | 190907 | 139763703 | −6.00E−04 | NA | NA | NA | NA | NA |
| WCM162_Z9_1 | 8 | 139767450 | 146156846 | 0.168 | NA | NA | NA | NA | NA |
| WCM162_Z9_1 | 8 | 146157063 | 146279528 | 0.006 | NA | NA | NA | NA | NA |
| WCM162_Z9_1 | 9 | 17056 | 328102 | −0.2616 | NA | NA | NA | NA | NA |
| WCM162_Z9_1 | 9 | 332403 | 95608515 | 0.0992 | NA | NA | NA | NA | NA |
| WCM162_Z9_1 | 9 | 95608796 | 141111414 | 0.0011 | NA | NA | NA | NA | NA |
| WCM162_Z9_1 | X | 2700119 | 45059985 | −0.0831 | NA | NA | NA | NA | NA |
| WCM162_Z9_1 | X | 46322830 | 63410486 | 0.259 | NA | NA | NA | NA | NA |
| WCM162_Z9_1 | X | 63410609 | 67273643 | 0.8022 | NA | NA | NA | NA | NA |
| WCM162_Z9_1 | X | 67283750 | 67741339 | 0.119 | NA | NA | NA | NA | NA |
| WCM162_Z9_1 | X | 67742672 | 154509348 | −0.0596 | NA | NA | NA | NA | NA |
| WCM163 | 1 | 14907 | 3566483 | 0.0123 | 0.5873 | 1.7744 | 1.2303 | 2 | 1 |
| WCM163 | 1 | 3598910 | 19666149 | −0.435 | 0.0612 | 1.791 | 0.2957 | 2 | 0 |
| WCM163 | 1 | 19669362 | 20879690 | 0.067 | 0.6498 | 1.6315 | 1.5064 | 2 | 2 |
| WCM163 | 1 | 20880587 | 23857025 | −0.4016 | 0.1016 | 1.8536 | 0.2923 | 2 | 0 |
| WCM163 | 1 | 23884369 | 35447738 | −0.0429 | 0.5238 | 1.7792 | 1.0964 | 2 | 1 |
| WCM163 | 1 | 35449342 | 53736722 | −0.2013 | 0.3396 | 1.7747 | 0.7561 | 2 | 1 |
| WCM163 | 1 | 53736989 | 142713878 | −0.0229 | 0.5469 | 1.797 | 1.1249 | 2 | 1 |
| WCM163 | 1 | 142727161 | 249213961 | 0.501 | 1.1354 | 2.4746 | 1.9189 | 2 | 2 |
| WCM163 | 10 | 225920 | 32576111 | −0.3522 | 0.1609 | 1.1233 | 1.1126 | 1 | 1 |
| WCM163 | 10 | 32580219 | 135477928 | −0.0707 | 0.4918 | 1.5135 | 1.2989 | 2 | 1 |
| WCM163 | 11 | 193105 | 62297210 | −0.0347 | 0.5333 | 1.7806 | 1.1139 | 2 | 1 |
| WCM163 | 11 | 62297548 | 65233864 | −0.1497 | 0.4 | 1.6458 | 0.9933 | 2 | 1 |
| WCM163 | 11 | 65265442 | 65293083 | −1.1962 | −0.928 | NA | NA | NA | NA |
| WCM163 | 11 | 65293528 | 67022750 | −0.1661 | 0.3809 | 1.5015 | 1.1028 | 2 | 1 |
| WCM163 | 11 | 67023037 | 134605916 | −0.0296 | 0.5391 | 1.7174 | 1.1888 | 2 | 1 |
| WCM163 | 12 | 148427 | 38364209 | −0.5394 | −0.0661 | 1.8691 | 0.0413 | 2 | 0 |
| WCM163 | 12 | 38596734 | 39998777 | −0.1326 | 0.42 | NA | NA | NA | NA |
| WCM163 | 12 | 40001483 | 54719023 | −0.2378 | 0.2968 | 1.6251 | 0.8317 | 2 | 1 |
| WCM163 | 12 | 54731126 | 133810883 | −0.1111 | 0.445 | 1.5557 | 1.1669 | 2 | 1 |
| WCM163 | 13 | 19042059 | 22178191 | 0.2662 | 0.8748 | 2.341 | 1.3266 | 2 | 1 |
| WCM163 | 13 | 22202744 | 24334368 | −0.618 | −0.1634 | 1.3135 | 0.4724 | 1 | 0 |
| WCM163 | 13 | 24334651 | 32849014 | 0.3807 | 1.0024 | 2.0478 | 1.9589 | 2 | 2 |
| WCM163 | 13 | 32849172 | 44971311 | −0.4337 | 0.0628 | 1.693 | 0.3961 | 2 | 0 |
| WCM163 | 13 | 45008046 | 48664499 | −0.8988 | −0.5225 | 0.9663 | 0.426 | 1 | 0 |
| WCM163 | 13 | 48667727 | 50140708 | −1.8694 | −2.013 | 0.3361 | 0.1594 | 0 | 0 |
| WCM163 | 13 | 50140962 | 51523661 | −1.0341 | −0.7033 | NA | NA | NA | NA |
| WCM163 | 13 | 51528055 | 72012167 | −0.5312 | −0.056 | 1.5384 | 0.3854 | 2 | 0 |
| WCM163 | 13 | 72012603 | 96539554 | 0.1089 | 0.6975 | 1.6549 | 1.5886 | 2 | 2 |
| WCM163 | 13 | 96540242 | 101256476 | −0.1908 | 0.352 | 1.2764 | 1.2764 | 1 | 1 |
| WCM163 | 13 | 101257253 | 115089411 | 0.1536 | 0.7481 | 1.6986 | 1.6606 | 2 | 2 |
| WCM163 | 14 | 19110338 | 51491970 | 0.2016 | 0.8022 | 1.7438 | 1.7438 | 2 | 2 |
| WCM163 | 14 | 51560857 | 71582083 | 0.0076 | 0.5818 | 1.7697 | 1.2239 | 2 | 1 |
| WCM163 | 14 | 71996051 | 107283270 | 0.2769 | 0.8867 | 1.849 | 1.849 | 2 | 2 |
| WCM163 | 15 | 20083968 | 23114146 | −0.1674 | 0.3794 | 1.8648 | 0.7368 | 2 | 1 |
| WCM163 | 15 | 23128382 | 59344625 | −0.0092 | 0.5626 | 1.6482 | 1.3056 | 2 | 1 |
| WCM163 | 15 | 59344672 | 102506240 | −0.2043 | 0.3362 | 1.7084 | 0.8163 | 2 | 1 |
| WCM163 | 16 | 64430 | 103575 | −0.535 | −0.0607 | NA | NA | NA | NA |
| WCM163 | 16 | 103923 | 71319679 | −0.0598 | 0.5044 | 1.4513 | 1.3857 | 1 | 1 |
| WCM163 | 16 | 71392787 | 90168687 | −0.6674 | −0.2251 | 1.651 | 0.06 | 2 | 0 |
| WCM163 | 17 | 131595 | 463759 | −0.0487 | 0.5172 | NA | NA | NA | NA |
| WCM163 | 17 | 463843 | 7478498 | −0.3709 | 0.1384 | 1.1007 | 1.1007 | 1 | 1 |
| WCM163 | 17 | 7480081 | 7960992 | −0.7263 | −0.2996 | 1.6402 | −0.0152 | 2 | 0 |
| WCM163 | 17 | 7976291 | 81052551 | 0.0475 | 0.6275 | 1.7443 | 1.3456 | 2 | 1 |
| WCM163 | 18 | 47666 | 78005146 | 0.2038 | 0.8048 | 1.7469 | 1.7469 | 2 | 2 |
| WCM163 | 19 | 104935 | 30054822 | 0.125 | 0.7158 | 1.6424 | 1.6424 | 2 | 2 |
| WCM163 | 19 | 30094208 | 43699356 | −0.2697 | 0.259 | 2.3409 | 0.0524 | 2 | 0 |
| WCM163 | 19 | 43702293 | 43766263 | −1.9045 | −2.0799 | NA | NA | NA | NA |
| WCM163 | 19 | 43772124 | 54006848 | −0.3115 | 0.2095 | 2.2816 | 0.0309 | 2 | 0 |
| WCM163 | 19 | 54072622 | 59093696 | 0.3796 | 1.0012 | 2.0327 | 1.9705 | 2 | 2 |
| WCM163 | 2 | 41662 | 96850652 | −0.043 | 0.5238 | 1.4377 | 1.4377 | 1 | 1 |
| WCM163 | 2 | 96850885 | 96971102 | −0.3593 | 0.1523 | NA | NA | NA | NA |
| WCM163 | 2 | 96991492 | 233470843 | −0.1367 | 0.4151 | 1.4903 | 1.1766 | 1 | 1 |
| WCM163 | 2 | 233498576 | 242690876 | −0.2234 | 0.3137 | 1.5529 | 0.9328 | 2 | 1 |
| WCM163 | 2 | 242694601 | 243037013 | −0.0441 | 0.5224 | 1.6252 | 1.2476 | 2 | 1 |
| WCM163 | 20 | 68336 | 62959246 | 0.016 | 0.5915 | 1.6812 | 1.3324 | 2 | 1 |
| WCM163 | 21 | 9590326 | 39906149 | −0.0053 | 0.5671 | 1.6976 | 1.2655 | 2 | 1 |
| WCM163 | 21 | 39947601 | 42848578 | −0.5563 | −0.0869 | 1.8449 | 0.0382 | 2 | 0 |
| WCM163 | 21 | 42851197 | 48084149 | 0.0467 | 0.6267 | 1.6851 | 1.4029 | 2 | 1 |
| WCM163 | 22 | 16158973 | 25774702 | −0.064 | 0.4995 | 1.6162 | 1.2112 | 2 | 1 |
| WCM163 | 22 | 25777515 | 50870685 | −0.5582 | −0.0893 | 1.0165 | 0.8635 | 1 | 1 |
| WCM163 | 22 | 50873364 | 51223730 | 0.051 | 0.6315 | 2.1789 | 0.9195 | 2 | 1 |
| WCM163 | 3 | 239349 | 46784329 | 0.0175 | 0.5933 | 1.7247 | 1.2927 | 2 | 1 |
| WCM163 | 3 | 46785355 | 52821011 | −0.0884 | 0.4713 | 1.7204 | 1.0523 | 2 | 1 |
| WCM163 | 3 | 52821186 | 71161672 | −0.0185 | 0.552 | 1.6936 | 1.2385 | 2 | 1 |
| WCM163 | 3 | 71179844 | 75496082 | −0.5217 | −0.0443 | 1.9212 | 0.0183 | 2 | 0 |
| WCM163 | 3 | 75599058 | 101062749 | 0.0256 | 0.6025 | 1.7374 | 1.2994 | 2 | 1 |
| WCM163 | 3 | 101066612 | 115342611 | −0.1257 | 0.428 | 1.7882 | 0.9025 | 2 | 1 |
| WCM163 | 3 | 115350247 | 141682610 | 0.0246 | 0.6014 | 1.7777 | 1.2567 | 2 | 1 |
| WCM163 | 3 | 141688904 | 160970552 | −0.2444 | 0.289 | 1.7608 | 0.6827 | 2 | 1 |
| WCM163 | 3 | 161062777 | 192053227 | −0.0258 | 0.5435 | 1.7275 | 1.1875 | 2 | 1 |
| WCM163 | 3 | 192078416 | 197785570 | −0.1981 | 0.3434 | 1.7941 | 0.7434 | 2 | 1 |
| WCM163 | 3 | 197785804 | 197896656 | 0.0263 | 0.6033 | NA | NA | NA | NA |
| WCM163 | 4 | 53469 | 52779488 | −0.4711 | 0.0175 | 1.5418 | 0.4826 | 2 | 0 |
| WCM163 | 4 | 52779778 | 90855914 | −0.2035 | 0.3371 | 1.4975 | 1.0289 | 1 | 1 |
| WCM163 | 4 | 90856341 | 184647831 | −0.0726 | 0.4896 | 1.7287 | 1.0793 | 2 | 1 |
| WCM163 | 4 | 184827253 | 186298102 | 0.3807 | 1.0024 | 3.9816 | 0.025 | 4 | 0 |
| WCM163 | 4 | 186299319 | 190903980 | −0.3908 | 0.1146 | 2.1036 | 0.0618 | 2 | 0 |
| WCM163 | 5 | 90680 | 50138236 | 0.0176 | 0.5934 | 1.6989 | 1.3187 | 2 | 1 |
| WCM163 | 5 | 50679150 | 148618941 | −0.3623 | 0.1488 | 1.8192 | 0.3981 | 2 | 0 |
| WCM163 | 5 | 148619412 | 180687804 | 0.0587 | 0.6403 | 1.7721 | 1.3453 | 2 | 1 |
| WCM163 | 6 | 203364 | 10430494 | −0.041 | 0.526 | 1.8017 | 1.0782 | 2 | 1 |
| WCM163 | 6 | 10509342 | 31584223 | 0.3541 | 0.9729 | 1.9758 | 1.9496 | 2 | 2 |
| WCM163 | 6 | 31584512 | 31829856 | 1.00E−04 | 0.5733 | 1.5801 | 1.3957 | 2 | 1 |
| WCM163 | 6 | 31830485 | 47846043 | 0.2179 | 0.8207 | 1.7662 | 1.7662 | 2 | 2 |
| WCM163 | 6 | 47846349 | 70890113 | 0.0763 | 0.6604 | 1.7804 | 1.3808 | 2 | 1 |
| WCM163 | 6 | 70890394 | 84563410 | −0.0181 | 0.5524 | 1.5053 | 1.4277 | 2 | 1 |
| WCM163 | 6 | 84563915 | 142487469 | −0.2636 | 0.2663 | 1.2041 | 1.2013 | 1 | 1 |
| WCM163 | 6 | 142490556 | 167275697 | 0.0371 | 0.6157 | 1.809 | 1.2555 | 2 | 1 |
| WCM163 | 6 | 167343104 | 170893464 | −0.2774 | 0.2499 | 1.2117 | 1.1666 | 1 | 1 |
| WCM163 | 7 | 193346 | 87908854 | −0.0517 | 0.5137 | 1.7011 | 1.1542 | 2 | 1 |
| WCM163 | 7 | 87908918 | 129908822 | −0.2153 | 0.3233 | 1.7365 | 0.7658 | 2 | 1 |
| WCM163 | 7 | 129909527 | 131150990 | −0.1831 | 0.361 | 1.5114 | 1.0572 | 2 | 1 |
| WCM163 | 7 | 131155668 | 144094425 | 0.0293 | 0.6067 | 1.7998 | 1.2458 | 2 | 1 |
| WCM163 | 7 | 144094600 | 158937135 | −0.2131 | 0.3258 | 1.7394 | 0.7673 | 2 | 1 |
| WCM163 | 8 | 163536 | 48974169 | −0.0426 | 0.5241 | 1.4497 | 1.4265 | 1 | 1 |
| WCM163 | 8 | 49503215 | 63161660 | 0.1983 | 0.7986 | 1.7394 | 1.7394 | 2 | 2 |
| WCM163 | 8 | 63195064 | 100865937 | 0.4437 | 1.0721 | 2.1397 | 2.0654 | 2 | 2 |
| WCM163 | 8 | 100866202 | 145616288 | 0.2879 | 0.8991 | 2.2984 | 1.4313 | 2 | 1 |
| WCM163 | 8 | 145616586 | 146279480 | 0.1178 | 0.7076 | 1.8943 | 1.3719 | 2 | 1 |
| WCM163 | 9 | 15900 | 141107582 | 0.2001 | 0.8006 | 1.7418 | 1.7418 | 2 | 2 |
| WCM163 | X | 2700156 | 64254635 | 0.5579 | 1.1977 | NA | NA | NA | NA |
| WCM163 | X | 64435187 | 67333003 | 1.1805 | 1.868 | NA | NA | NA | NA |
| WCM163 | X | 67339047 | 154509348 | 0.5898 | 1.2327 | NA | NA | NA | NA |
| WCM163_Z7_1 | 1 | 69592 | 3566525 | 0.3303 | 0.9981 | 2.037 | 1.9578 | 2 | 2 |
| WCM163_Z7_1 | 1 | 3598963 | 8029446 | −0.6118 | −0.003 | 2.0112 | −0.0154 | 2 | 0 |
| WCM163_Z7_1 | 1 | 8031068 | 8482842 | 0.1934 | 0.855 | NA | NA | NA | NA |
| WCM163_Z7_1 | 1 | 8525970 | 19655128 | −0.5865 | 0.0244 | 2.0317 | 0.0025 | 2 | 0 |
| WCM163_Z7_1 | 1 | 19666094 | 20834410 | 0.2761 | 0.9415 | NA | NA | NA | NA |
| WCM163_Z7_1 | 1 | 20879475 | 23857070 | −0.6031 | 0.0064 | 1.9856 | 0.0233 | 2 | 0 |
| WCM163_Z7_1 | 1 | 23857285 | 34684351 | 0.3398 | 1.008 | 2.0321 | 1.9902 | 2 | 2 |
| WCM163_Z7_1 | 1 | 35135208 | 46775884 | −0.6139 | −0.0053 | 2.0016 | −0.009 | 2 | 0 |
| WCM163_Z7_1 | 1 | 46782226 | 47025935 | 0.2573 | 0.9218 | NA | NA | NA | NA |
| WCM163_Z7_1 | 1 | 47027285 | 53793521 | −0.611 | −0.0021 | 2.0015 | −0.0045 | 2 | 0 |
| WCM163_Z7_1 | 1 | 53925247 | 64680553 | 0.3529 | 1.0217 | 2.0493 | 2.0112 | 2 | 2 |
| WCM163_Z7_1 | 1 | 64686564 | 67880900 | −0.6106 | −0.0017 | 1.9999 | −0.0023 | 2 | 0 |
| WCM163_Z7_1 | 1 | 67885713 | 114646337 | 0.3348 | 1.0028 | 2.029 | 1.9788 | 2 | 2 |
| WCM163_Z7_1 | 1 | 114680276 | 116675831 | −0.5851 | 0.026 | NA | NA | NA | NA |
| WCM163_Z7_1 | 1 | 116916851 | 120611489 | 0.3059 | 0.9726 | 2.0771 | 1.8476 | 2 | 2 |
| WCM163_Z7_1 | 1 | 120611968 | 249212557 | 0.8618 | 1.5489 | 3.9322 | 1.9196 | 4 | 2 |
| WCM163_Z7_1 | 10 | 225980 | 135440080 | −0.0721 | 0.5754 | 1.9963 | 0.9839 | 2 | 1 |
| WCM163_Z7_1 | 11 | 193106 | 134856534 | −0.0869 | 0.5597 | 1.9516 | 0.9963 | 2 | 1 |
| WCM163_Z7_1 | 12 | 148674 | 12879241 | −0.5995 | 0.0104 | 2.0237 | −0.0093 | 2 | 0 |
| WCM163_Z7_1 | 12 | 12939872 | 33592436 | −1.4442 | −0.9371 | 1.0402 | 0.0044 | 1 | 0 |
| WCM163_Z7_1 | 12 | 34175539 | 133810735 | −0.6064 | 0.0029 | 1.014 | 0.99 | 1 | 1 |
| WCM163_Z7_1 | 13 | 19600515 | 22275541 | 0.2854 | 0.9512 | NA | NA | NA | NA |
| WCM163_Z7_1 | 13 | 23238161 | 24334681 | −0.5944 | 0.0159 | 2.0542 | −0.0321 | 2 | 0 |
| WCM163_Z7_1 | 13 | 24380087 | 32841457 | 0.2931 | 0.9593 | 2.0093 | 1.8793 | 2 | 2 |
| WCM163_Z7_1 | 13 | 32849042 | 48919320 | −0.5891 | 0.0216 | 2.0114 | 0.0188 | 2 | 0 |
| WCM163_Z7_1 | 13 | 48947628 | 49865887 | −3.4303 | −3.9035 | NA | NA | NA | NA |
| WCM163_Z7_1 | 13 | 49885021 | 70681598 | −0.5821 | 0.0292 | 2.0315 | 0.0094 | 2 | 0 |
| WCM163_Z7_1 | 13 | 72014800 | 115089411 | −0.0633 | 0.5847 | 2.0043 | 0.9951 | 2 | 1 |
| WCM163_Z7_1 | 14 | 19377719 | 71576774 | −0.0784 | 0.5687 | 1.9667 | 0.9996 | 2 | 1 |
| WCM163_Z7_1 | 14 | 72040288 | 107283184 | 0.3162 | 0.9834 | 1.9936 | 1.9606 | 2 | 2 |
| WCM163_Z7_1 | 15 | 20169912 | 59523960 | −0.0685 | 0.5792 | 2 | 0.9881 | 2 | 1 |
| WCM163_Z7_1 | 15 | 59528860 | 102506276 | −0.6131 | −0.0045 | 1.0142 | 0.9795 | 1 | 1 |
| WCM163_Z7_1 | 16 | 66966 | 71319801 | −0.0827 | 0.5642 | 1.9795 | 0.9776 | 2 | 1 |
| WCM163_Z7_1 | 16 | 71392741 | 90162182 | −1.4646 | −0.9609 | 1.0286 | −0.0011 | 1 | 0 |
| WCM163_Z7_1 | 17 | 131595 | 7478703 | −0.0759 | 0.5714 | 1.9846 | 0.9873 | 2 | 1 |
| WCM163_Z7_1 | 17 | 7479880 | 7977032 | −0.7388 | −0.1417 | 1.8343 | −0.0214 | 2 | 0 |
| WCM163_Z7_1 | 17 | 7978932 | 81083526 | 0.3122 | 0.9793 | 1.9818 | 1.9612 | 2 | 2 |
| WCM163_Z7_1 | 18 | 49039 | 32864519 | 0.3018 | 0.9684 | 1.9811 | 1.9321 | 2 | 2 |
| WCM163_Z7_1 | 18 | 32864994 | 78005210 | −0.0741 | 0.5733 | 2.0297 | 0.9462 | 2 | 1 |
| WCM163_Z7_1 | 19 | 281499 | 24310731 | 0.3097 | 0.9766 | 1.9842 | 1.9514 | 2 | 2 |
| WCM163_Z7_1 | 19 | 29567134 | 53995279 | −0.6375 | −0.031 | 1.9592 | −0.0017 | 2 | 0 |
| WCM163_Z7_1 | 19 | 54072640 | 59083968 | 0.2712 | 0.9364 | 1.9257 | 1.9017 | 2 | 2 |
| WCM163_Z7_1 | 2 | 41614 | 95537463 | 0.3203 | 0.9877 | 2.9662 | 0.9998 | 3 | 1 |
| WCM163_Z7_1 | 2 | 95537770 | 127839416 | −0.5952 | 0.015 | 1.0335 | 0.9874 | 1 | 1 |
| WCM163_Z7_1 | 2 | 127944890 | 128284004 | −1.4578 | −0.953 | NA | NA | NA | NA |
| WCM163_Z7_1 | 2 | 128284225 | 216986843 | −0.6124 | −0.0037 | 1.0169 | 0.9779 | 1 | 1 |
| WCM163_Z7_1 | 2 | 216990755 | 220505600 | −0.0958 | 0.5503 | 1.9809 | 0.9478 | 2 | 1 |
| WCM163_Z7_1 | 2 | 220506380 | 242946616 | −0.6091 | −1.00E−04 | 1.0173 | 0.9826 | 1 | 1 |
| WCM163_Z7_1 | 20 | 68351 | 62904890 | 0.3046 | 0.9713 | 1.9709 | 1.9504 | 2 | 2 |
| WCM163_Z7_1 | 21 | 9826225 | 39818748 | 0.2806 | 0.9463 | 1.951 | 1.9027 | 2 | 2 |
| WCM163_Z7_1 | 21 | 39947601 | 42866383 | −0.6125 | −0.0038 | 2.0036 | −0.0089 | 2 | 0 |
| WCM163_Z7_1 | 21 | 42870049 | 48084214 | 0.297 | 0.9633 | 1.9742 | 1.9253 | 2 | 2 |
| WCM163_Z7_1 | 22 | 17063440 | 50893487 | −0.609 | 0 | 1.0794 | 0.9207 | 1 | 1 |
| WCM163_Z7_1 | 22 | 50893569 | 51215115 | −0.0999 | 0.5459 | 1.8575 | 1.0624 | 2 | 1 |
| WCM163_Z7_1 | 3 | 239377 | 46063329 | −0.066 | 0.5819 | 1.9608 | 1.0327 | 2 | 1 |
| WCM163_Z7_1 | 3 | 46244879 | 52730331 | −0.5985 | 0.0114 | 2.013 | 0.0028 | 2 | 0 |
| WCM163_Z7_1 | 3 | 52730673 | 71247064 | −0.0357 | 0.6138 | 2.0333 | 1.0273 | 2 | 1 |
| WCM163_Z7_1 | 3 | 71247462 | 75478292 | −0.5856 | 0.0254 | NA | NA | NA | NA |
| WCM163_Z7_1 | 3 | 75832483 | 197894720 | −0.0784 | 0.5687 | 1.9652 | 1.0012 | 2 | 1 |
| WCM163_Z7_1 | 4 | 59327 | 49063842 | −0.0703 | 0.5773 | 1.9815 | 1.0025 | 2 | 1 |
| WCM163_Z7_1 | 4 | 52709803 | 98480213 | −0.5934 | 0.017 | 2.0315 | −0.0078 | 2 | 0 |
| WCM163_Z7_1 | 4 | 98633927 | 100470508 | 0.2958 | 0.9621 | NA | NA | NA | NA |
| WCM163_Z7_1 | 4 | 100472128 | 110880530 | −0.6432 | −0.0372 | 1.9989 | −0.0498 | 2 | 0 |
| WCM163_Z7_1 | 4 | 110882078 | 184633769 | −0.073 | 0.5744 | 1.9682 | 1.01 | 2 | 1 |
| WCM163_Z7_1 | 4 | 184827965 | 186337006 | 0.5587 | 1.2357 | 4.687 | 0.0229 | 5 | 0 |
| WCM163_Z7_1 | 4 | 186339611 | 187348117 | −0.4995 | 0.1187 | 2.1845 | −0.0131 | 2 | 0 |
| WCM163_Z7_1 | 4 | 187455127 | 190396320 | −0.6291 | −0.0218 | NA | NA | NA | NA |
| WCM163_Z7_1 | 5 | 140532 | 9630067 | 0.3068 | 0.9736 | 2.015 | 1.9125 | 2 | 2 |
| WCM163_Z7_1 | 5 | 10227673 | 11903945 | −0.6293 | −0.0221 | NA | NA | NA | NA |
| WCM163_Z7_1 | 5 | 13692218 | 50129872 | 0.2997 | 0.9662 | 1.9536 | 1.9536 | 2 | 2 |
| WCM163_Z7_1 | 5 | 50130675 | 148639132 | −0.6038 | 0.0057 | 1.9967 | 0.0112 | 2 | 0 |
| WCM163_Z7_1 | 5 | 148679086 | 180687808 | 0.3126 | 0.9796 | 1.972 | 1.972 | 2 | 2 |
| WCM163_Z7_1 | 6 | 203619 | 10415173 | −0.1098 | 0.5354 | 1.9026 | 0.9961 | 2 | 1 |
| WCM163_Z7_1 | 6 | 10419680 | 31503233 | 0.2873 | 0.9532 | 1.9584 | 1.9139 | 2 | 2 |
| WCM163_Z7_1 | 6 | 31540784 | 75977432 | −0.0754 | 0.5719 | 1.9931 | 0.9798 | 2 | 1 |
| WCM163_Z7_1 | 6 | 75994283 | 76332511 | −1.4163 | −0.9045 | NA | NA | NA | NA |
| WCM163_Z7_1 | 6 | 76333621 | 170893415 | −0.0767 | 0.5705 | 1.9834 | 0.9867 | 2 | 1 |
| WCM163_Z7_1 | 7 | 193254 | 72400606 | −0.0776 | 0.5696 | 1.9664 | 1.0018 | 2 | 1 |
| WCM163_Z7_1 | 7 | 72409165 | 131060212 | 0.3162 | 0.9834 | 2.004 | 1.9503 | 2 | 2 |
| WCM163_Z7_1 | 7 | 131071891 | 144096925 | −0.0881 | 0.5584 | 1.9639 | 0.9814 | 2 | 1 |
| WCM163_Z7_1 | 7 | 144097354 | 158937013 | 0.3074 | 0.9743 | 1.9954 | 1.9339 | 2 | 2 |
| WCM163_Z7_1 | 8 | 190907 | 62626884 | −0.0854 | 0.5613 | 1.9647 | 0.9865 | 2 | 1 |
| WCM163_Z7_1 | 8 | 63161664 | 146279480 | 0.3115 | 0.9785 | 1.989 | 1.952 | 2 | 2 |
| WCM163_Z7_1 | 9 | 116846 | 131493649 | 0.3095 | 0.9764 | 1.9819 | 1.9531 | 2 | 2 |
| WCM163_Z7_1 | 9 | 131495750 | 132816234 | −0.0901 | 0.5563 | 1.9751 | 0.9659 | 2 | 1 |
| WCM163_Z7_1 | 9 | 132837968 | 141111414 | 0.3364 | 1.0045 | 2.0272 | 1.9854 | 2 | 2 |
| WCM163_Z7_1 | X | 2707716 | 154509348 | 0.2148 | 0.8774 | NA | NA | NA | NA |
| WCM164 | 1 | 14930 | 249213967 | −0.033 | −0.1813 | 0.8819 | 0.8819 | 1 | 1 |
| WCM164 | 10 | 226100 | 135439019 | 0.0077 | 0.3635 | 1.2866 | 1.2866 | 1 | 1 |
| WCM164 | 11 | 193096 | 134605959 | −0.011 | 0.1362 | 1.099 | 1.099 | 1 | 1 |
| WCM164 | 12 | 148414 | 133810703 | −0.0165 | 0.0629 | 1.0445 | 1.0445 | 1 | 1 |
| WCM164 | 13 | 19041992 | 115089411 | −0.1247 | −6.8365 | 0.0088 | 0.0088 | 0 | 0 |
| WCM164 | 14 | 19110263 | 107283270 | −0.0591 | −0.6714 | 0.6279 | 0.6279 | 1 | 1 |
| WCM164 | 15 | 20083896 | 102506237 | −0.0226 | −0.0225 | 0.9845 | 0.9845 | 1 | 1 |
| WCM164 | 16 | 66930 | 90168646 | −0.0362 | −0.2338 | 0.8504 | 0.8504 | 1 | 1 |
| WCM164 | 17 | 131595 | 81187875 | −0.0633 | −0.7671 | 0.5876 | 0.5876 | 1 | 1 |
| WCM164 | 18 | 47610 | 78005197 | −0.082 | −1.2916 | 0.4085 | 0.4085 | 0 | 0 |
| WCM164 | 19 | 105050 | 844077 | 0.1202 | 1.3041 | 3.6351 | 1.3034 | 4 | 1 |
| WCM164 | 19 | 846272 | 19166817 | −0.0842 | −1.3657 | 0.388 | 0.388 | 0 | 0 |
| WCM164 | 19 | 19168336 | 59093425 | 0.0653 | 0.9115 | 1.881 | 1.881 | 2 | 2 |
| WCM164 | 2 | 41602 | 243037022 | −0.0453 | −0.3932 | 0.7614 | 0.7614 | 1 | 1 |
| WCM164 | 20 | 68406 | 62934572 | 0.0126 | 0.4183 | 1.3363 | 1.3363 | 1 | 1 |
| WCM164 | 21 | 9590368 | 48084204 | −0.0375 | −0.2555 | 0.8377 | 0.8377 | 1 | 1 |
| WCM164 | 22 | 16159079 | 51219129 | −0.027 | −0.0882 | 0.9407 | 0.9407 | 1 | 1 |
| WCM164 | 3 | 239524 | 197896705 | −0.0347 | −0.2093 | 0.8649 | 0.8649 | 1 | 1 |
| WCM164 | 4 | 53428 | 190878479 | −0.019 | 0.0284 | 1.0199 | 1.0199 | 1 | 1 |
| WCM164 | 5 | 90696 | 180687776 | −0.0325 | −0.1744 | 0.8861 | 0.8861 | 1 | 1 |
| WCM164 | 6 | 203374 | 26368360 | −0.0682 | −0.8888 | 0.5401 | 0.5401 | 1 | 1 |
| WCM164 | 6 | 26370634 | 86195006 | 0.0355 | 0.6512 | 1.5705 | 1.5705 | 2 | 2 |
| WCM164 | 6 | 86195167 | 170892848 | −0.1243 | −6.3259 | 0.0125 | 0.0125 | 0 | 0 |
| WCM164 | 7 | 193253 | 158937046 | 0.0377 | 0.6723 | 1.5936 | 1.5936 | 2 | 2 |
| WCM164 | 8 | 163506 | 42231674 | −0.0862 | −1.4404 | 0.3685 | 0.3685 | 0 | 0 |
| WCM164 | 8 | 42231845 | 144657760 | 0.2013 | 1.7569 | 3.3797 | 3.3797 | 3 | 3 |
| WCM164 | 8 | 144658262 | 146279547 | 0.0333 | 0.6301 | 2.2152 | 0.8801 | 2 | 1 |
| WCM164 | 9 | 116774 | 141107495 | −0.0298 | −0.1314 | 0.913 | 0.913 | 1 | 1 |
| WCM164 | X | 2700110 | 154509348 | −0.0465 | −0.4148 | NA | NA | NA | NA |
| WCM164_X1_1 | 1 | 69635 | 2408142 | −0.1825 | 0.6373 | 1.9209 | 1.19 | 2 | 1 |
| WCM164_X1_1 | 1 | 2410049 | 20680456 | −0.8557 | −0.731 | 1.1041 | 0.1009 | 1 | 0 |
| WCM164_X1_1 | 1 | 20680600 | 249212402 | −0.1868 | 0.6302 | 2.014 | 1.0815 | 2 | 1 |
| WCM164_X1_1 | 10 | 255939 | 84745256 | 0.2678 | 1.3171 | 2.928 | 2.0551 | 3 | 2 |
| WCM164_X1_1 | 10 | 85901270 | 88446833 | −0.1692 | 0.6591 | 2.8068 | 0.3515 | 3 | 0 |
| WCM164_X1_1 | 10 | 88451790 | 90354519 | 0.1921 | 1.2098 | 2.6079 | 2.0182 | 3 | 2 |
| WCM164_X1_1 | 10 | 90356656 | 101124261 | 0.4675 | 1.5897 | 3.8318 | 2.1878 | 4 | 2 |
| WCM164_X1_1 | 10 | 101124720 | 101515420 | 0.3837 | 1.477 | NA | NA | NA | NA |
| WCM164_X1_1 | 10 | 101542607 | 103384550 | −0.4903 | 0.0901 | 1.9934 | 0.1354 | 2 | 0 |
| WCM164_X1_1 | 10 | 103427711 | 121612629 | 0.4507 | 1.5673 | 3.7919 | 2.1352 | 4 | 2 |
| WCM164_X1_1 | 10 | 121616863 | 121663703 | 0.1085 | 1.0884 | NA | NA | NA | NA |
| WCM164_X1_1 | 10 | 121668592 | 124214448 | −0.5586 | −0.0455 | 1.9692 | −0.0313 | 2 | 0 |
| WCM164_X1_1 | 10 | 124214532 | 135440086 | 0.0514 | 1.0037 | 2.0232 | 1.9871 | 2 | 2 |
| WCM164_X1_1 | 11 | 193152 | 17868245 | 0.0382 | 0.9838 | 2.21 | 1.7454 | 2 | 2 |
| WCM164_X1_1 | 11 | 17940305 | 18436773 | −0.5186 | 0.0348 | 1.9192 | 0.1296 | 2 | 0 |
| WCM164_X1_1 | 11 | 18456308 | 134856536 | 0.0321 | 0.9747 | 1.9771 | 1.9533 | 2 | 2 |
| WCM164_X1_1 | 12 | 148644 | 26383903 | 0.2891 | 1.3468 | 2.9085 | 2.1786 | 3 | 2 |
| WCM164_X1_1 | 12 | 26405818 | 133780851 | 0.0607 | 1.0177 | 2.0519 | 1.9975 | 2 | 2 |
| WCM164_X1_1 | 12 | 133781072 | 133810703 | 0.1941 | 1.2127 | NA | NA | NA | NA |
| WCM164_X1_1 | 13 | 19600515 | 115089411 | −0.4838 | 0.1026 | 2.1126 | 0.0348 | 2 | 0 |
| WCM164_X1_1 | 14 | 19377692 | 20470519 | −0.1082 | 0.7573 | 2.2123 | 1.1684 | 2 | 1 |
| WCM164_X1_1 | 14 | 20470737 | 63749879 | −0.1589 | 0.6758 | 2.0157 | 1.1793 | 2 | 1 |
| WCM164_X1_1 | 14 | 63757641 | 65569057 | −0.4822 | 0.1058 | 2.1473 | 0.0048 | 2 | 0 |
| WCM164_X1_1 | 14 | 65734340 | 71444717 | 0.0645 | 1.0233 | 2.1054 | 1.9598 | 2 | 2 |
| WCM164_X1_1 | 14 | 71445100 | 73538368 | −0.5329 | 0.0063 | NA | NA | NA | NA |
| WCM164_X1_1 | 14 | 73543046 | 107283203 | 0.0808 | 1.0476 | 2.0917 | 2.0426 | 2 | 2 |
| WCM164_X1_1 | 15 | 20170150 | 41772425 | −0.1607 | 0.6729 | 2.0301 | 1.1584 | 2 | 1 |
| WCM164_X1_1 | 15 | 41772812 | 43699658 | −0.8487 | −0.7123 | 1.0661 | 0.1546 | 1 | 0 |
| WCM164_X1_1 | 15 | 43700203 | 102506273 | −0.1869 | 0.6301 | 1.9502 | 1.1451 | 2 | 1 |
| WCM164_X1_1 | 16 | 67604 | 72170625 | 0.0725 | 1.0352 | 2.0677 | 2.0311 | 2 | 2 |
| WCM164_X1_1 | 16 | 72173279 | 74334954 | −0.7697 | −0.5125 | NA | NA | NA | NA |
| WCM164_X1_1 | 16 | 74335498 | 75564150 | 0.0994 | 1.0751 | 2.2188 | 1.9951 | 2 | 2 |
| WCM164_X1_1 | 16 | 75565504 | 83983920 | −0.4964 | 0.0782 | 2.0198 | 0.0916 | 2 | 0 |
| WCM164_X1_1 | 16 | 83984894 | 84651466 | 0.0922 | 1.0645 | 2.1996 | 1.9832 | 2 | 2 |
| WCM164_X1_1 | 16 | 84684517 | 85138998 | −0.4833 | 0.1037 | 2.1097 | 0.0393 | 2 | 0 |
| WCM164_X1_1 | 16 | 85141491 | 88933013 | 0.1411 | 1.1362 | 2.2652 | 2.1309 | 2 | 2 |
| WCM164_X1_1 | 16 | 88943648 | 90233622 | 0.4383 | 1.5507 | 3.7548 | 2.1044 | 4 | 2 |
| WCM164_X1_1 | 17 | 131595 | 21191309 | −0.4944 | 0.0822 | 2.0344 | 0.0828 | 2 | 0 |
| WCM164_X1_1 | 17 | 21194843 | 27067924 | 0.1691 | 1.1767 | 2.9258 | 1.5955 | 3 | 2 |
| WCM164_X1_1 | 17 | 27068131 | 74527752 | −0.1808 | 0.64 | 2.0701 | 1.0467 | 2 | 1 |
| WCM164_X1_1 | 17 | 74533540 | 81083455 | 0.2775 | 1.3306 | 2.9439 | 2.0863 | 3 | 2 |
| WCM164_X1_1 | 18 | 48238 | 78005197 | −0.4816 | 0.107 | 2.0947 | 0.0593 | 2 | 0 |
| WCM164_X1_1 | 19 | 281498 | 2015331 | 0.1007 | 1.0771 | 2.3368 | 1.8827 | 2 | 2 |
| WCM164_X1_1 | 19 | 2039707 | 14591479 | −0.5021 | 0.0671 | 2.0017 | 0.0934 | 2 | 0 |
| WCM164_X1_1 | 19 | 14593667 | 16686944 | 0.4727 | 1.5966 | 4.0746 | 1.9739 | 4 | 2 |
| WCM164_X1_1 | 19 | 16687339 | 17327069 | 0.0751 | 1.0391 | NA | NA | NA | NA |
| WCM164_X1_1 | 19 | 17328276 | 18562382 | −0.5189 | 0.0342 | 1.9469 | 0.101 | 2 | 0 |
| WCM164_X1_1 | 19 | 18569116 | 34934811 | 0.0645 | 1.0233 | 2.1023 | 1.9628 | 2 | 2 |
| WCM164_X1_1 | 19 | 34935949 | 35614212 | −0.4385 | 0.1889 | 2.1627 | 0.117 | 2 | 0 |
| WCM164_X1_1 | 19 | 35616191 | 42497555 | 0.0588 | 1.0148 | 2.2592 | 1.782 | 2 | 2 |
| WCM164_X1_1 | 19 | 42525461 | 42799297 | −0.4973 | 0.0764 | NA | NA | NA | NA |
| WCM164_X1_1 | 19 | 42801345 | 59083953 | 0.4384 | 1.5508 | 3.8455 | 2.0142 | 4 | 2 |
| WCM164_X1_1 | 2 | 41615 | 242946597 | −0.1861 | 0.6314 | 2.0232 | 1.075 | 2 | 1 |
| WCM164_X1_1 | 20 | 68396 | 62904847 | −0.151 | 0.6887 | 1.9357 | 1.2879 | 2 | 1 |
| WCM164_X1_1 | 21 | 9826203 | 10910347 | −0.5671 | −0.0629 | NA | NA | NA | NA |
| WCM164_X1_1 | 21 | 10914363 | 17566741 | −0.4362 | 0.1933 | NA | NA | NA | NA |
| WCM164_X1_1 | 21 | 17763960 | 39817521 | 0.0757 | 1.0401 | 2.1144 | 1.9982 | 2 | 2 |
| WCM164_X1_1 | 21 | 39818748 | 42817383 | −0.503 | 0.0653 | 1.9815 | 0.1111 | 2 | 0 |
| WCM164_X1_1 | 21 | 42817930 | 48084277 | 0.1161 | 1.0996 | 2.2309 | 2.0549 | 2 | 2 |
| WCM164_X1_1 | 22 | 16287446 | 39267732 | 0.2807 | 1.335 | 3.0266 | 2.019 | 3 | 2 |
| WCM164_X1_1 | 22 | 39353703 | 51215120 | −0.1875 | 0.6291 | 2.0508 | 1.0423 | 2 | 1 |
| WCM164_X1_1 | 3 | 239350 | 4759080 | 0.0494 | 1.0008 | 2.2081 | 1.794 | 2 | 2 |
| WCM164_X1_1 | 3 | 4767244 | 4942294 | −1.2976 | −2.4761 | NA | NA | NA | NA |
| WCM164_X1_1 | 3 | 5021403 | 9857817 | −0.5046 | 0.0622 | 2.0558 | 0.0322 | 2 | 0 |
| WCM164_X1_1 | 3 | 9859380 | 46509745 | 0.0625 | 1.0203 | 2.0284 | 2.0284 | 2 | 2 |
| WCM164_X1_1 | 3 | 46539556 | 65607679 | 0.2835 | 1.339 | 2.6603 | 2.3991 | 3 | 2 |
| WCM164_X1_1 | 3 | 66023739 | 73114621 | −0.4847 | 0.1009 | 2.0734 | 0.0714 | 2 | 0 |
| WCM164_X1_1 | 3 | 73432623 | 123550262 | 0.2828 | 1.3379 | 2.8454 | 2.2104 | 3 | 2 |
| WCM164_X1_1 | 3 | 123616407 | 124998008 | −0.2022 | 0.6048 | 2.9475 | 0.094 | 3 | 0 |
| WCM164_X1_1 | 3 | 125006994 | 175181248 | 0.2747 | 1.3267 | 2.803 | 2.2136 | 3 | 2 |
| WCM164_X1_1 | 3 | 175184925 | 176768376 | 0.2039 | 1.2267 | NA | NA | NA | NA |
| WCM164_X1_1 | 3 | 176769343 | 197894712 | −0.1895 | 0.6258 | 2.9926 | 0.0935 | 3 | 0 |
| WCM164_X1_1 | 4 | 59334 | 190426502 | 0.0641 | 1.0227 | 2.056 | 2.0075 | 2 | 2 |
| WCM164_X1_1 | 5 | 140532 | 88100493 | 0.0184 | 0.954 | 1.9549 | 1.9197 | 2 | 2 |
| WCM164_X1_1 | 5 | 89689917 | 108523200 | −0.5568 | −0.0419 | 1.9255 | 0.0172 | 2 | 0 |
| WCM164_X1_1 | 5 | 108673043 | 132763351 | 0.0083 | 0.9386 | 1.9896 | 1.8438 | 2 | 2 |
| WCM164_X1_1 | 5 | 132918994 | 134130562 | −0.575 | −0.0789 | NA | NA | NA | NA |
| WCM164_X1_1 | 5 | 134131770 | 180687766 | 0.0153 | 0.9493 | 1.9514 | 1.9104 | 2 | 2 |
| WCM164_X1_1 | 6 | 203479 | 25520513 | −0.5032 | 0.0649 | 2.0322 | 0.0598 | 2 | 0 |
| WCM164_X1_1 | 6 | 25529050 | 33556747 | 0.069 | 1.03 | 2.0755 | 2.0086 | 2 | 2 |
| WCM164_X1_1 | 6 | 33560992 | 38950227 | 0.7641 | 1.9727 | 5.7719 | 2.0781 | 6 | 2 |
| WCM164_X1_1 | 6 | 38951998 | 47277181 | 1.0476 | 2.3203 | 7.9738 | 2.0152 | 8 | 2 |
| WCM164_X1_1 | 6 | 47471020 | 57372307 | −0.178 | 0.6446 | 2.0298 | 1.0968 | 2 | 1 |
| WCM164_X1_1 | 6 | 57398163 | 74537868 | 0.2213 | 1.2514 | 3.0737 | 1.6876 | 3 | 2 |
| WCM164_X1_1 | 6 | 75794916 | 76564953 | 0.7477 | 1.9521 | NA | NA | NA | NA |
| WCM164_X1_1 | 6 | 76570740 | 107956440 | 0.2741 | 1.3258 | 2.9485 | 2.0648 | 3 | 2 |
| WCM164_X1_1 | 6 | 107956582 | 170892708 | −0.4815 | 0.1072 | 2.1365 | 0.0178 | 2 | 0 |
| WCM164_X1_1 | 7 | 193254 | 71275347 | −0.1855 | 0.6323 | 2.0549 | 1.0451 | 2 | 1 |
| WCM164_X1_1 | 7 | 71488741 | 123672171 | 0.247 | 1.2878 | 3.801 | 1.0822 | 4 | 1 |
| WCM164_X1_1 | 7 | 123672860 | 131071908 | 0.0582 | 1.0139 | 3.9729 | 0.0658 | 4 | 0 |
| WCM164_X1_1 | 7 | 131073716 | 151855968 | 0.2673 | 1.3163 | 3.8933 | 1.0872 | 4 | 1 |
| WCM164_X1_1 | 7 | 151859240 | 152345741 | −0.7929 | −0.5695 | NA | NA | NA | NA |
| WCM164_X1_1 | 7 | 152346092 | 158937022 | 0.0837 | 1.0519 | 3.0536 | 1.0929 | 3 | 1 |
| WCM164_X1_1 | 8 | 190907 | 28420424 | −0.4827 | 0.1048 | 2.0196 | 0.1311 | 2 | 0 |
| WCM164_X1_1 | 8 | 28573618 | 67417505 | 0.4749 | 1.5996 | 4.0584 | 2.0028 | 4 | 2 |
| WCM164_X1_1 | 8 | 67422365 | 143746111 | 0.772 | 1.9827 | 5.9035 | 2.0011 | 6 | 2 |
| WCM164_X1_1 | 8 | 143781858 | 146279441 | 0.4641 | 1.5852 | 5.9009 | 0.1001 | 6 | 0 |
| WCM164_X1_1 | 9 | 17085 | 43884984 | −0.4779 | 0.1141 | 2.0464 | 0.1182 | 2 | 0 |
| WCM164_X1_1 | 9 | 44870008 | 77112899 | 0.0453 | 0.9946 | NA | NA | NA | NA |
| WCM164_X1_1 | 9 | 77230548 | 84208159 | −0.4969 | 0.0772 | 1.99 | 0.12 | 2 | 0 |
| WCM164_X1_1 | 9 | 84215027 | 128725347 | 0.0785 | 1.0441 | 2.0621 | 2.0621 | 2 | 2 |
| WCM164_X1_1 | 9 | 128728183 | 141111414 | −0.184 | 0.6349 | 2.0532 | 1.0525 | 2 | 1 |
| WCM164_X1_1 | X | 2707687 | 154509348 | 0.044 | 0.9926 | NA | NA | NA | NA |
| WCM169 | 1 | 14930 | 249213973 | 0.0218 | 0.3259 | 1.2535 | 1.2535 | 1 | 1 |
| WCM169 | 10 | 226091 | 135438982 | 0.0084 | 0.1339 | 1.0973 | 1.0973 | 1 | 1 |
| WCM169 | 11 | 193096 | 62327934 | 0.0387 | 0.539 | 1.453 | 1.453 | 1 | 1 |
| WCM169 | 11 | 62334423 | 71150131 | −0.0842 | −4.1943 | 0.0546 | 0.0546 | 0 | 0 |
| WCM169 | 11 | 71152328 | 134605916 | 0.0178 | 0.271 | 1.2067 | 1.2067 | 1 | 1 |
| WCM169 | 12 | 148637 | 133810883 | −0.0085 | −0.1484 | 0.9022 | 0.9022 | 1 | 1 |
| WCM169 | 13 | 19041937 | 115089411 | −0.0228 | −0.4372 | 0.7386 | 0.7386 | 1 | 1 |
| WCM169 | 14 | 19110239 | 107283270 | −0.0093 | −0.1637 | 0.8927 | 0.8927 | 1 | 1 |
| WCM169 | 15 | 20083896 | 102506238 | 0.0226 | 0.3366 | 1.2628 | 1.2628 | 1 | 1 |
| WCM169 | 16 | 64401 | 90168646 | −0.0015 | −0.0257 | 0.9823 | 0.9823 | 1 | 1 |
| WCM169 | 17 | 131595 | 81187934 | −8.00E−04 | −0.0137 | 0.9906 | 0.9906 | 1 | 1 |
| WCM169 | 18 | 47711 | 78005203 | −0.0159 | −0.2906 | 0.8176 | 0.8176 | 1 | 1 |
| WCM169 | 19 | 105069 | 59093645 | −0.0155 | −0.2837 | 0.8215 | 0.8215 | 1 | 1 |
| WCM169 | 2 | 41585 | 243037039 | −0.0035 | −0.0595 | 0.9596 | 0.9596 | 1 | 1 |
| WCM169 | 20 | 68427 | 62934623 | 0.0229 | 0.3404 | 1.2661 | 1.2661 | 1 | 1 |
| WCM169 | 21 | 9590334 | 48084177 | −0.0077 | −0.1339 | 0.9114 | 0.9114 | 1 | 1 |
| WCM169 | 22 | 16159060 | 51215157 | 0.0184 | 0.279 | 1.2134 | 1.2134 | 1 | 1 |
| WCM169 | 3 | 239451 | 197896711 | −0.0048 | −0.0824 | 0.9445 | 0.9445 | 1 | 1 |
| WCM169 | 4 | 53359 | 190903980 | −0.0067 | −0.1165 | 0.9224 | 0.9224 | 1 | 1 |
| WCM169 | 5 | 90680 | 180687808 | −0.0107 | −0.1896 | 0.8769 | 0.8769 | 1 | 1 |
| WCM169 | 6 | 203561 | 170892869 | −0.0082 | −0.1438 | 0.9051 | 0.9051 | 1 | 1 |
| WCM169 | 7 | 193253 | 158937022 | 2.00E−04 | 0.004 | 1.0028 | 1.0028 | 1 | 1 |
| WCM169 | 8 | 163438 | 146279483 | −0.0099 | −0.1751 | 0.8857 | 0.8857 | 1 | 1 |
| WCM169 | 9 | 14886 | 141071710 | 0.0173 | 0.2641 | 1.2009 | 1.2009 | 1 | 1 |
| WCM169 | X | 2700151 | 154493590 | 0.0333 | 0.4745 | NA | NA | NA | NA |
| WCM169_X1_1 | 1 | 69603 | 249212164 | −0.0365 | 0.064 | 1.2262 | 0.8645 | 1 | 1 |
| WCM169_X1_1 | 10 | 225979 | 135440080 | −0.0983 | −0.68 | 0.7012 | 0.5472 | 1 | 1 |
| WCM169_X1_1 | 11 | 193154 | 134856604 | −0.0621 | −0.2033 | 1.0659 | 0.6712 | 1 | 1 |
| WCM169_X1_1 | 12 | 148811 | 133810842 | −0.0677 | −0.269 | 1.0265 | 0.6333 | 1 | 1 |
| WCM169_X1_1 | 13 | 19600515 | 115089411 | −0.091 | −0.5721 | 0.721 | 0.6243 | 1 | 1 |
| WCM169_X1_1 | 14 | 19377711 | 107283034 | −0.0303 | 0.1219 | 1.1992 | 0.9772 | 1 | 1 |
| WCM169_X1_1 | 15 | 20170156 | 102506273 | −0.0327 | 0.0997 | 1.3032 | 0.8399 | 1 | 1 |
| WCM169_X1_1 | 16 | 66930 | 90161580 | 0.0052 | 0.4219 | 1.4328 | 1.2467 | 1 | 1 |
| WCM169_X1_1 | 17 | 131595 | 81083455 | 0.0252 | 0.5698 | 1.5578 | 1.411 | 2 | 1 |
| WCM169_X1_1 | 18 | 48201 | 78005164 | −0.0492 | −0.0635 | 1.0366 | 0.8773 | 1 | 1 |
| WCM169_X1_1 | 19 | 281404 | 59083985 | 0.0118 | 0.4722 | 1.5638 | 1.2107 | 2 | 1 |
| WCM169_X1_1 | 2 | 41614 | 242946597 | −0.0485 | −0.0563 | 1.0315 | 0.892 | 1 | 1 |
| WCM169_X1_1 | 20 | 68397 | 62904925 | 0.0157 | 0.5011 | 1.5195 | 1.3112 | 2 | 1 |
| WCM169_X1_1 | 21 | 9826225 | 48084214 | −0.034 | 0.0876 | 1.133 | 0.9921 | 1 | 1 |
| WCM169_X1_1 | 22 | 17063476 | 51215123 | 0.0201 | 0.5334 | 1.5518 | 1.3428 | 2 | 1 |
| WCM169_X1_1 | 3 | 239405 | 197896714 | −0.0132 | 0.2731 | 1.2798 | 1.1369 | 1 | 1 |
| WCM169_X1_1 | 4 | 59345 | 190426502 | −0.1152 | −0.966 | 0.607 | 0.4168 | 1 | 0 |
| WCM169_X1_1 | 5 | 140538 | 180687776 | −0.0911 | −0.5729 | 0.74 | 0.6046 | 1 | 1 |
| WCM169_X1_1 | 6 | 203699 | 170893450 | −0.0609 | −0.1896 | 1.0744 | 0.6793 | 1 | 1 |
| WCM169_X1_1 | 7 | 193253 | 158937135 | −0.0942 | −0.6183 | 1.2884 | 0.0144 | 1 | 0 |
| WCM169_X1_1 | 8 | 190907 | 146279434 | −0.1115 | −0.8992 | 0.6316 | 0.4408 | 1 | 0 |
| WCM169_X1_1 | 9 | 17084 | 141111414 | −0.087 | −0.515 | 0.7677 | 0.6319 | 1 | 1 |
| WCM169_X1_1 | X | 2707716 | 154509348 | −0.0992 | −0.6945 | NA | NA | NA | NA |
| WCM170 | 1 | 17465 | 249214018 | −0.0186 | 0.1415 | 1.103 | 1.103 | 1 | 1 |
| WCM170 | 10 | 225982 | 135439032 | −0.0374 | −0.1253 | 0.9168 | 0.9168 | 1 | 1 |
| WCM170 | 11 | 193096 | 134320514 | −0.0018 | 0.3467 | 1.2716 | 1.2716 | 1 | 1 |
| WCM170 | 12 | 148444 | 133810832 | 0.0256 | 0.633 | 1.5508 | 1.5508 | 2 | 2 |
| WCM170 | 13 | 19042005 | 115089411 | −0.0793 | −0.9703 | 0.5104 | 0.5104 | 1 | 1 |
| WCM170 | 14 | 19110239 | 107283156 | −0.0481 | −0.3008 | 0.8118 | 0.8118 | 1 | 1 |
| WCM170 | 15 | 20083896 | 102506273 | −0.0287 | 0.0044 | 1.0031 | 1.0031 | 1 | 1 |
| WCM170 | 16 | 67688 | 90260461 | −0.0503 | −0.3398 | 0.7902 | 0.7902 | 1 | 1 |
| WCM170 | 17 | 131595 | 81052563 | −0.0641 | −0.6074 | 0.6564 | 0.6564 | 1 | 1 |
| WCM170 | 18 | 47682 | 78005201 | −0.0881 | −1.2295 | 0.4265 | 0.4265 | 0 | 0 |
| WCM170 | 19 | 105069 | 59093696 | −0.0427 | −0.2089 | 0.8652 | 0.8652 | 1 | 1 |
| WCM170 | 2 | 41474 | 243037103 | −0.0232 | 0.0808 | 1.0576 | 1.0576 | 1 | 1 |
| WCM170 | 20 | 68319 | 62934623 | 0.0091 | 0.4676 | 1.3828 | 1.3828 | 1 | 1 |
| WCM170 | 21 | 9590370 | 48084189 | −0.0405 | −0.1742 | 0.8862 | 0.8862 | 1 | 1 |
| WCM170 | 22 | 16159014 | 51219090 | −0.0296 | −0.0084 | 0.9942 | 0.9942 | 1 | 1 |
| WCM170 | 3 | 239436 | 197896656 | −0.0245 | 0.0634 | 1.0449 | 1.0449 | 1 | 1 |
| WCM170 | 4 | 53360 | 190878508 | −0.0965 | −1.5264 | 0.3471 | 0.3471 | 0 | 0 |
| WCM170 | 5 | 90680 | 45261219 | 0.0542 | 0.8857 | 1.8476 | 1.8476 | 2 | 2 |
| WCM170 | 5 | 45261346 | 140957127 | −0.1334 | −8.5453 | 0.0027 | 0.0027 | 0 | 0 |
| WCM170 | 5 | 140957746 | 180687821 | −0.0151 | 0.1864 | 1.1379 | 1.1379 | 1 | 1 |
| WCM170 | 6 | 203545 | 170892848 | −0.0581 | −0.4847 | 0.7146 | 0.7146 | 1 | 1 |
| WCM170 | 7 | 193222 | 158937011 | 0.0277 | 0.6532 | 1.5727 | 1.5727 | 2 | 2 |
| WCM170 | 8 | 163527 | 146279544 | 0.0295 | 0.6701 | 1.5912 | 1.5912 | 2 | 2 |
| WCM170 | 9 | 116855 | 141107532 | 0.0229 | 0.6069 | 1.523 | 1.523 | 2 | 2 |
| WCM170 | X | 2700125 | 154493609 | −0.0128 | 0.2152 | NA | NA | NA | NA |
| WCM170_Z1_1 | 1 | 69603 | 249212557 | −0.0642 | −0.0388 | 0.9869 | 0.9601 | 1 | 1 |
| WCM170_Z1_1 | 10 | 225980 | 135440068 | −0.0831 | −0.0914 | 0.9518 | 0.9254 | 1 | 1 |
| WCM170_Z1_1 | 11 | 193113 | 77409733 | −0.0397 | 0.0277 | 1.199 | 0.8397 | 1 | 1 |
| WCM170_Z1_1 | 11 | 77412026 | 134856542 | 0.1901 | 0.5748 | 1.997 | 0.9819 | 2 | 1 |
| WCM170_Z1_1 | 12 | 148644 | 38714925 | −0.0156 | 0.0912 | 1.1261 | 1.0043 | 1 | 1 |
| WCM170_Z1_1 | 12 | 39047837 | 73015527 | 0.2944 | 0.7897 | 2.4596 | 0.9978 | 2 | 1 |
| WCM170_Z1_1 | 12 | 73046114 | 117365827 | 0.4683 | 1.1165 | 3.3875 | 0.9488 | 3 | 1 |
| WCM170_Z1_1 | 12 | 117383210 | 133810703 | 0.3011 | 0.803 | 2.4756 | 1.0139 | 2 | 1 |
| WCM170_Z1_1 | 13 | 19600365 | 115089411 | −0.1036 | −0.1496 | 0.9249 | 0.878 | 1 | 1 |
| WCM170_Z1_1 | 14 | 19377692 | 107283027 | −0.0787 | −0.0788 | 0.976 | 0.9177 | 1 | 1 |
| WCM170_Z1_1 | 15 | 20170150 | 57839587 | −0.0406 | 0.0252 | 1.1944 | 0.8408 | 1 | 1 |
| WCM170_Z1_1 | 15 | 57891898 | 63009804 | −0.244 | −0.5996 | 1.0852 | 0.2347 | 1 | 0 |
| WCM170_Z1_1 | 15 | 63012010 | 77908028 | −0.0478 | 0.0058 | 1.042 | 0.9662 | 1 | 1 |
| WCM170_Z1_1 | 15 | 77926631 | 78894604 | −0.2811 | −0.7372 | 1.1571 | 0.0427 | 1 | 0 |
| WCM170_Z1_1 | 15 | 78909398 | 84287031 | −0.0403 | 0.026 | 1.1243 | 0.9121 | 1 | 1 |
| WCM170_Z1_1 | 15 | 84324566 | 89074314 | −0.3875 | −1.1966 | 1.078 | −0.2054 | 1 | 0 |
| WCM170_Z1_1 | 15 | 89074587 | 102506273 | −0.0407 | 0.025 | 1.0556 | 0.9794 | 1 | 1 |
| WCM170_Z1_1 | 16 | 67604 | 90233599 | −0.0471 | 0.0079 | 1.0271 | 0.9838 | 1 | 1 |
| WCM170_Z1_1 | 17 | 131595 | 7214789 | −0.0783 | −0.0778 | 0.982 | 0.913 | 1 | 1 |
| WCM170_Z1_1 | 17 | 7214960 | 19705108 | −0.3654 | −1.0917 | 1.06 | −0.1216 | 1 | 0 |
| WCM170_Z1_1 | 17 | 19708017 | 54872439 | −0.0812 | −0.0859 | 0.966 | 0.9184 | 1 | 1 |
| WCM170_Z1_1 | 17 | 54892235 | 56811549 | −0.3402 | −0.9784 | 0.8634 | 0.1517 | 1 | 0 |
| WCM170_Z1_1 | 17 | 56833502 | 81083455 | −0.0597 | −0.0264 | 0.9926 | 0.9711 | 1 | 1 |
| WCM170_Z1_1 | 18 | 48266 | 78005164 | −0.331 | −0.9386 | 1.1135 | −0.07 | 1 | 0 |
| WCM170_Z1_1 | 19 | 281424 | 3767338 | −0.3088 | −0.8465 | 1.044 | 0.0682 | 1 | 0 |
| WCM170_Z1_1 | 19 | 3770696 | 42728723 | −0.0507 | −0.0018 | 1.0231 | 0.9744 | 1 | 1 |
| WCM170_Z1_1 | 19 | 42728836 | 46997897 | −0.3518 | −1.0297 | 1.0379 | −0.0583 | 1 | 0 |
| WCM170_Z1_1 | 19 | 46998236 | 59083923 | −0.0641 | −0.0384 | 1.0085 | 0.9389 | 1 | 1 |
| WCM170_Z1_1 | 2 | 41614 | 103149100 | −0.0571 | −0.0194 | 1.0082 | 0.9651 | 1 | 1 |
| WCM170_Z1_1 | 2 | 103236360 | 112917368 | 0.2656 | 0.7319 | 1.8525 | 1.4691 | 2 | 1 |
| WCM170_Z1_1 | 2 | 112922704 | 234959741 | −0.0697 | −0.0539 | 0.99 | 0.9367 | 1 | 1 |
| WCM170_Z1_1 | 2 | 234967560 | 242946604 | −0.3549 | −1.0435 | 1.0321 | −0.0618 | 1 | 0 |
| WCM170_Z1_1 | 20 | 68373 | 33122595 | −0.0273 | 0.0605 | 1.084 | 1.0016 | 1 | 1 |
| WCM170_Z1_1 | 20 | 33122709 | 62904863 | 0.2571 | 0.7147 | 2.1227 | 1.1597 | 2 | 1 |
| WCM170_Z1_1 | 21 | 9826225 | 48084282 | −0.0692 | −0.0525 | 1.0283 | 0.9003 | 1 | 1 |
| WCM170_Z1_1 | 22 | 16287461 | 51215142 | −0.063 | −0.0355 | 1.0078 | 0.9435 | 1 | 1 |
| WCM170_Z1_1 | 3 | 239405 | 70014274 | −0.0426 | 0.02 | 1.0303 | 0.9977 | 1 | 1 |
| WCM170_Z1_1 | 3 | 71007463 | 88199313 | −0.3566 | −1.051 | NA | NA | NA | NA |
| WCM170_Z1_1 | 3 | 88202415 | 172353810 | −0.0378 | 0.0328 | 1.0448 | 1.0012 | 1 | 1 |
| WCM170_Z1_1 | 3 | 172363447 | 197894726 | 0.1801 | 0.5533 | 1.9271 | 1.0078 | 2 | 1 |
| WCM170_Z1_1 | 4 | 59391 | 66217296 | −0.0829 | −0.0907 | 0.9813 | 0.8968 | 1 | 1 |
| WCM170_Z1_1 | 4 | 66218767 | 190426471 | −0.3788 | −1.1544 | 0.9571 | −0.0586 | 1 | 0 |
| WCM170_Z1_1 | 5 | 140538 | 45695959 | 0.239 | 0.6775 | 2.2499 | 0.9488 | 2 | 1 |
| WCM170_Z1_1 | 5 | 49698142 | 133537659 | −0.2795 | −0.731 | 0.9759 | 0.229 | 1 | 0 |
| WCM170_Z1_1 | 5 | 133541598 | 180687812 | 0.1058 | 0.3876 | 2.3018 | 0.3146 | 2 | 0 |
| WCM170_Z1_1 | 6 | 203577 | 165895824 | −0.0586 | −0.0234 | 1.0108 | 0.9571 | 1 | 1 |
| WCM170_Z1_1 | 6 | 165956988 | 168695345 | −0.338 | −0.9687 | 0.8519 | 0.1701 | 1 | 0 |
| WCM170_Z1_1 | 6 | 168708354 | 170893406 | −0.0861 | −0.0998 | 1.0702 | 0.7961 | 1 | 1 |
| WCM170_Z1_1 | 7 | 193254 | 14026288 | 0.1243 | 0.4299 | 1.4356 | 1.2587 | 1 | 1 |
| WCM170_Z1_1 | 7 | 14027756 | 66774614 | 0.3354 | 0.87 | 1.8806 | 1.7747 | 2 | 2 |
| WCM170_Z1_1 | 7 | 69064936 | 158937416 | 0.0677 | 0.2983 | 1.9921 | 0.4673 | 2 | 0 |
| WCM170_Z1_1 | 8 | 190900 | 39873099 | −0.2016 | −0.4532 | 1.5844 | −0.1236 | 2 | 0 |
| WCM170_Z1_1 | 8 | 40011281 | 134108453 | 0.3644 | 0.9254 | 2.3026 | 1.4958 | 2 | 1 |
| WCM170_Z1_1 | 8 | 134125682 | 146279545 | 0.1139 | 0.4062 | 1.413 | 1.2374 | 1 | 1 |
| WCM170_Z1_1 | 9 | 116832 | 5361821 | −0.201 | −0.4512 | 0.8726 | 0.5902 | 1 | 1 |
| WCM170_Z1_1 | 9 | 5456150 | 7105406 | −0.5581 | −2.339 | NA | NA | NA | NA |
| WCM170_Z1_1 | 9 | 7128104 | 66457076 | −0.1034 | −0.1491 | 0.9383 | 0.8653 | 1 | 1 |
| WCM170_Z1_1 | 9 | 66499220 | 141111414 | 0.0806 | 0.329 | 2.0521 | 0.4602 | 2 | 0 |
| WCM170_Z1_1 | X | 2700125 | 154509348 | 0.3432 | 0.885 | NA | NA | NA | NA |
| WCM170_Z16_1 | 1 | 69592 | 234041378 | −0.0348 | 0.0367 | 1.0297 | 1.0218 | 1 | 1 |
| WCM170_Z16_1 | 1 | 234350315 | 237063481 | −0.7925 | −1.0249 | 0.9308 | 0.0521 | 1 | 0 |
| WCM170_Z16_1 | 1 | 237430905 | 249212557 | −0.1042 | −0.0524 | 1.0192 | 0.9095 | 1 | 1 |
| WCM170_Z16_1 | 10 | 225982 | 65370001 | −0.0802 | −0.0214 | 0.9853 | 0.9853 | 1 | 1 |
| WCM170_Z16_1 | 10 | 65379544 | 71634885 | 0.3827 | 0.5505 | 1.8594 | 1.0699 | 2 | 1 |
| WCM170_Z16_1 | 10 | 71637810 | 83635724 | 0.7225 | 0.9476 | 2.8483 | 1.009 | 3 | 1 |
| WCM170_Z16_1 | 10 | 83635914 | 89420748 | −0.118 | −0.0702 | 0.971 | 0.934 | 1 | 1 |
| WCM170_Z16_1 | 10 | 89463512 | 91484799 | −0.8471 | −1.1118 | NA | NA | NA | NA |
| WCM170_Z16_1 | 10 | 91486160 | 135440108 | −0.1102 | −0.0601 | 0.9592 | 0.9592 | 1 | 1 |
| WCM170_Z16_1 | 11 | 193100 | 3661375 | −0.0557 | 0.01 | 1.1874 | 0.8265 | 1 | 1 |
| WCM170_Z16_1 | 11 | 3661536 | 4929430 | 0.5129 | 0.7046 | 2.1057 | 1.1537 | 2 | 1 |
| WCM170_Z16_1 | 11 | 4936253 | 6292743 | −0.0306 | 0.042 | 1.0296 | 1.0296 | 1 | 1 |
| WCM170_Z16_1 | 11 | 6340480 | 7111436 | 0.6767 | 0.8949 | 2.7204 | 0.9986 | 3 | 1 |
| WCM170_Z16_1 | 11 | 7256057 | 134856603 | −0.0555 | 0.0102 | 1.0174 | 0.9968 | 1 | 1 |
| WCM170_Z16_1 | 12 | 148811 | 17142456 | −0.0481 | 0.0197 | 1.0384 | 0.9891 | 1 | 1 |
| WCM170_Z16_1 | 12 | 17826313 | 33031940 | 0.5411 | 0.7376 | 2.3069 | 1.028 | 2 | 1 |
| WCM170_Z16_1 | 12 | 33049563 | 55969088 | −0.0478 | 0.0201 | 1.9887 | 0.0394 | 2 | 0 |
| WCM170_Z16_1 | 12 | 56030806 | 72863602 | 0.3805 | 0.548 | 1.9068 | 1.0173 | 2 | 1 |
| WCM170_Z16_1 | 12 | 72866880 | 78598891 | 1.0827 | 1.3536 | NA | NA | NA | NA |
| WCM170_Z16_1 | 12 | 78604205 | 89916595 | 0.6458 | 0.8593 | 3.6042 | 0.024 | 4 | 0 |
| WCM170_Z16_1 | 12 | 89917037 | 117476976 | 0.9835 | 1.243 | 3.7973 | 0.9365 | 4 | 1 |
| WCM170_Z16_1 | 12 | 117479762 | 130185206 | 0.7331 | 0.9596 | 2.8601 | 1.0296 | 3 | 1 |
| WCM170_Z16_1 | 12 | 130387852 | 133810703 | 0.371 | 0.5366 | 1.8456 | 1.0555 | 2 | 1 |
| WCM170_Z16_1 | 13 | 19600365 | 115089411 | −0.0613 | 0.0029 | 1.0174 | 0.9866 | 1 | 1 |
| WCM170_Z16_1 | 14 | 19377716 | 20403909 | −0.4742 | −0.5514 | NA | NA | NA | NA |
| WCM170_Z16_1 | 14 | 20404091 | 37754521 | −0.6779 | −0.8484 | 1.078 | 0.0328 | 1 | 0 |
| WCM170_Z16_1 | 14 | 37777274 | 79181460 | 0.4055 | 0.5777 | 1.968 | 1.0169 | 2 | 1 |
| WCM170_Z16_1 | 14 | 79270093 | 98218070 | −0.0854 | −0.0281 | 1.006 | 0.9555 | 1 | 1 |
| WCM170_Z16_1 | 14 | 98435700 | 107283213 | 0.4474 | 0.6274 | 2.0161 | 1.0735 | 2 | 1 |
| WCM170_Z16_1 | 15 | 20170150 | 54590067 | −0.6825 | −0.8554 | 1.0569 | 0.0485 | 1 | 0 |
| WCM170_Z16_1 | 15 | 54614222 | 57891880 | −0.132 | −0.0884 | NA | NA | NA | NA |
| WCM170_Z16_1 | 15 | 57896486 | 62948264 | −0.7616 | −0.9766 | 0.9776 | 0.0388 | 1 | 0 |
| WCM170_Z16_1 | 15 | 62950063 | 77770749 | 0.0078 | 0.0908 | 1.0824 | 1.0474 | 1 | 1 |
| WCM170_Z16_1 | 15 | 77771562 | 88799228 | −0.6989 | −0.8802 | 1.0484 | 0.0382 | 1 | 0 |
| WCM170_Z16_1 | 15 | 89003023 | 102506284 | −0.0236 | 0.0509 | 1.0742 | 0.9977 | 1 | 1 |
| WCM170_Z16_1 | 16 | 67604 | 90233599 | 0.0179 | 0.1035 | 1.0798 | 1.069 | 1 | 1 |
| WCM170_Z16_1 | 17 | 131595 | 7196815 | 0.0023 | 0.0838 | 1.0598 | 1.0598 | 1 | 1 |
| WCM170_Z16_1 | 17 | 7210413 | 19684318 | −0.6542 | −0.8129 | 1.1021 | 0.0363 | 1 | 0 |
| WCM170_Z16_1 | 17 | 19685315 | 49354579 | 0.0015 | 0.0829 | 1.0725 | 1.0457 | 1 | 1 |
| WCM170_Z16_1 | 17 | 49357388 | 57043188 | −0.5982 | −0.7299 | 1.1676 | 0.0383 | 1 | 0 |
| WCM170_Z16_1 | 17 | 57043561 | 81083455 | 0.034 | 0.1239 | 1.0979 | 1.0814 | 1 | 1 |
| WCM170_Z16_1 | 18 | 48221 | 78005197 | −0.668 | −0.8335 | 1.0696 | 0.0527 | 1 | 0 |
| WCM170_Z16_1 | 19 | 281404 | 3760211 | −0.6293 | −0.7758 | 1.1306 | 0.0375 | 1 | 0 |
| WCM170_Z16_1 | 19 | 3762752 | 42603950 | 0.0211 | 0.1076 | 1.0869 | 1.0679 | 1 | 1 |
| WCM170_Z16_1 | 19 | 42621155 | 47030244 | −0.6611 | −0.8232 | 1.0845 | 0.0458 | 1 | 0 |
| WCM170_Z16_1 | 19 | 47104178 | 59083985 | 0 | 0.081 | 1.0671 | 1.0484 | 1 | 1 |
| WCM170_Z16_1 | 2 | 41615 | 103149100 | −0.0242 | 0.0502 | 1.0512 | 1.0196 | 1 | 1 |
| WCM170_Z16_1 | 2 | 103236321 | 112834794 | 0.4101 | 0.5833 | 2.0198 | 0.9766 | 2 | 1 |
| WCM170_Z16_1 | 2 | 112838655 | 185803742 | −0.0374 | 0.0333 | 1.0338 | 1.0129 | 1 | 1 |
| WCM170_Z16_1 | 2 | 186603429 | 190044287 | −0.6636 | −0.8269 | 1.0289 | 0.0986 | 1 | 0 |
| WCM170_Z16_1 | 2 | 190306233 | 234969119 | −0.0359 | 0.0353 | 1.0352 | 1.0143 | 1 | 1 |
| WCM170_Z16_1 | 2 | 234975919 | 242946604 | −0.6511 | −0.8083 | 1.1358 | 0.0063 | 1 | 0 |
| WCM170_Z16_1 | 20 | 68351 | 36761253 | 0.0092 | 0.0926 | 1.0757 | 1.0568 | 1 | 1 |
| WCM170_Z16_1 | 20 | 36766633 | 62867958 | 0.4605 | 0.6429 | 2.0537 | 1.0693 | 2 | 1 |
| WCM170_Z16_1 | 20 | 62868665 | 62904881 | 0.4831 | 0.6695 | NA | NA | NA | NA |
| WCM170_Z16_1 | 21 | 9826230 | 36421143 | 0.0313 | 0.1205 | 1.1254 | 1.0488 | 1 | 1 |
| WCM170_Z16_1 | 21 | 36422352 | 48084226 | −0.6722 | −0.8399 | 1.069 | 0.0484 | 1 | 0 |
| WCM170_Z16_1 | 22 | 16287495 | 50832418 | 0.0147 | 0.0995 | 1.0809 | 1.0619 | 1 | 1 |
| WCM170_Z16_1 | 22 | 50845151 | 51215123 | −0.6146 | −0.754 | 1.1381 | 0.0478 | 1 | 0 |
| WCM170_Z16_1 | 3 | 239405 | 42954777 | −0.0223 | 0.0526 | 1.0477 | 1.0266 | 1 | 1 |
| WCM170_Z16_1 | 3 | 42956036 | 44673989 | −0.6719 | −0.8394 | NA | NA | NA | NA |
| WCM170_Z16_1 | 3 | 44683581 | 45772862 | −0.0122 | 0.0654 | 2.044 | 0.0487 | 2 | 0 |
| WCM170_Z16_1 | 3 | 45773610 | 88205677 | −0.6459 | −0.8005 | 1.0767 | 0.0716 | 1 | 0 |
| WCM170_Z16_1 | 3 | 89156905 | 113046535 | −0.0804 | −0.0218 | 1.0243 | 0.9457 | 1 | 1 |
| WCM170_Z16_1 | 3 | 113049242 | 114099624 | 0.6202 | 0.8297 | NA | NA | NA | NA |
| WCM170_Z16_1 | 3 | 114462303 | 149638994 | −0.0124 | 0.0652 | 1.0595 | 1.033 | 1 | 1 |
| WCM170_Z16_1 | 3 | 149677875 | 151599328 | −0.7169 | −0.9077 | 0.9805 | 0.0856 | 1 | 0 |
| WCM170_Z16_1 | 3 | 151986434 | 172163223 | −0.0657 | −0.0028 | 1.0288 | 0.9673 | 1 | 1 |
| WCM170_Z16_1 | 3 | 172165378 | 197894726 | 0.4512 | 0.6319 | 1.9691 | 1.1302 | 2 | 1 |
| WCM170_Z16_1 | 4 | 59331 | 67142617 | −0.0957 | −0.0414 | 0.988 | 0.9554 | 1 | 1 |
| WCM170_Z16_1 | 4 | 68355735 | 190426502 | −0.7517 | −0.9612 | 1.0038 | 0.0235 | 1 | 0 |
| WCM170_Z16_1 | 5 | 140515 | 45695803 | 0.3513 | 0.5131 | 1.8886 | 0.9656 | 2 | 1 |
| WCM170_Z16_1 | 5 | 49698142 | 133702132 | −0.7457 | −0.952 | 0.9931 | 0.0407 | 1 | 0 |
| WCM170_Z16_1 | 5 | 133707316 | 180687814 | −0.0555 | 0.0102 | 1.9972 | 0.017 | 2 | 0 |
| WCM170_Z16_1 | 6 | 203619 | 27215741 | −0.7136 | −0.9027 | 1.0218 | 0.048 | 1 | 0 |
| WCM170_Z16_1 | 6 | 27215807 | 45459757 | −0.054 | 0.0122 | 1.9648 | 0.0522 | 2 | 0 |
| WCM170_Z16_1 | 6 | 45480011 | 170893454 | −0.7342 | −0.9341 | 0.9929 | 0.0538 | 1 | 0 |
| WCM170_Z16_1 | 7 | 193222 | 14653016 | −0.067 | −0.0045 | 1.0084 | 0.9854 | 1 | 1 |
| WCM170_Z16_1 | 7 | 14661103 | 103629735 | 0.3705 | 0.536 | 1.9227 | 0.9771 | 2 | 1 |
| WCM170_Z16_1 | 7 | 103767338 | 158937416 | −0.0393 | 0.031 | 1.0374 | 1.0061 | 1 | 1 |
| WCM170_Z16_1 | 8 | 190908 | 40011239 | −0.66 | −0.8215 | 1.1136 | 0.0181 | 1 | 0 |
| WCM170_Z16_1 | 8 | 40011352 | 55534097 | 0.3598 | 0.5232 | 1.7225 | 1.1518 | 2 | 1 |
| WCM170_Z16_1 | 8 | 55534806 | 64100165 | −0.0586 | 0.0063 | NA | NA | NA | NA |
| WCM170_Z16_1 | 8 | 65290081 | 134239876 | 0.4108 | 0.584 | 1.9354 | 1.0627 | 2 | 1 |
| WCM170_Z16_1 | 8 | 134239949 | 146279453 | −0.0309 | 0.0417 | 1.0582 | 1.0005 | 1 | 1 |
| WCM170_Z16_1 | 9 | 116832 | 5361795 | 0.3325 | 0.4905 | 1.9651 | 0.8449 | 2 | 1 |
| WCM170_Z16_1 | 9 | 5456164 | 7046864 | −0.8022 | −1.0402 | NA | NA | NA | NA |
| WCM170_Z16_1 | 9 | 7049110 | 28888926 | 0.4154 | 0.5895 | 1.9031 | 1.1062 | 2 | 1 |
| WCM170_Z16_1 | 9 | 32384732 | 141111414 | −0.0731 | −0.0123 | 1.0017 | 0.9813 | 1 | 1 |
| WCM170_Z16_1 | X | 2700125 | 65483487 | −0.0788 | −0.0196 | NA | NA | NA | NA |
| WCM170_Z16_1 | X | 65486383 | 66943533 | 3.0196 | 3.4036 | NA | NA | NA | NA |
| WCM170_Z16_1 | X | 67268291 | 68840737 | 0.7674 | 0.9989 | NA | NA | NA | NA |
| WCM170_Z16_1 | X | 69176943 | 154509348 | −0.1139 | −0.0649 | NA | NA | NA | NA |
| WCM172 | 1 | 17473 | 249214028 | 0.0197 | NA | NA | NA | NA | NA |
| WCM172 | 10 | 225936 | 135477904 | −0.0057 | NA | NA | NA | NA | NA |
| WCM172 | 11 | 193146 | 134605954 | 0.0198 | NA | NA | NA | NA | NA |
| WCM172 | 12 | 148513 | 133810794 | −0.0117 | NA | NA | NA | NA | NA |
| WCM172 | 13 | 19041979 | 115089411 | −0.0318 | NA | NA | NA | NA | NA |
| WCM172 | 14 | 19110401 | 107283156 | 0.0095 | NA | NA | NA | NA | NA |
| WCM172 | 15 | 20083894 | 102515252 | 0.0082 | NA | NA | NA | NA | NA |
| WCM172 | 16 | 66870 | 90168646 | −0.0022 | NA | NA | NA | NA | NA |
| WCM172 | 17 | 131595 | 81187875 | 0.0172 | NA | NA | NA | NA | NA |
| WCM172 | 18 | 47716 | 78005205 | 0.0025 | NA | NA | NA | NA | NA |
| WCM172 | 19 | 104963 | 59093696 | −0.023 | NA | NA | NA | NA | NA |
| WCM172 | 2 | 41583 | 243037103 | −0.006 | NA | NA | NA | NA | NA |
| WCM172 | 20 | 68397 | 62934592 | 0.0349 | NA | NA | NA | NA | NA |
| WCM172 | 21 | 9590301 | 48084214 | 0.0236 | NA | NA | NA | NA | NA |
| WCM172 | 22 | 16159014 | 51219129 | 0.0424 | NA | NA | NA | NA | NA |
| WCM172 | 3 | 239436 | 197896705 | 0.0301 | NA | NA | NA | NA | NA |
| WCM172 | 4 | 53446 | 190878508 | 0.0272 | NA | NA | NA | NA | NA |
| WCM172 | 5 | 90680 | 180687803 | −0.0075 | NA | NA | NA | NA | NA |
| WCM172 | 6 | 203546 | 170892923 | −0.0135 | NA | NA | NA | NA | NA |
| WCM172 | 7 | 193253 | 158937158 | 0.0224 | NA | NA | NA | NA | NA |
| WCM172 | 8 | 163461 | 43048915 | −0.0455 | NA | NA | NA | NA | NA |
| WCM172 | 8 | 43052114 | 145999447 | 0.1836 | NA | NA | NA | NA | NA |
| WCM172 | 8 | 145999815 | 146279514 | 0.059 | NA | NA | NA | NA | NA |
| WCM172 | 9 | 15903 | 141107533 | 0.0299 | NA | NA | NA | NA | NA |
| WCM172 | X | 2700174 | 154507335 | −0.082 | NA | NA | NA | NA | NA |
| WCM172_X1_1 | 1 | 721476 | 160971123 | −0.1464 | 0.6446 | 2.045 | 1.0817 | 2 | 1 |
| WCM172_X1_1 | 1 | 160990858 | 178820272 | −0.3987 | 0.0863 | 2.1645 | −0.0412 | 2 | 0 |
| WCM172_X1_1 | 1 | 178821944 | 249212402 | 0.0584 | 1.0288 | 4.0409 | 0.0399 | 4 | 0 |
| WCM172_X1_1 | 10 | 255883 | 135440090 | −0.1567 | 0.624 | 1.9914 | 1.0909 | 2 | 1 |
| WCM172_X1_1 | 11 | 193112 | 79133220 | 0.0604 | 1.0325 | 2.0744 | 2.0166 | 2 | 2 |
| WCM172_X1_1 | 11 | 81601786 | 85340205 | −0.4127 | 0.0514 | NA | NA | NA | NA |
| WCM172_X1_1 | 11 | 85345210 | 134856603 | 0.0552 | 1.0231 | 2.0755 | 1.9892 | 2 | 2 |
| WCM172_X1_1 | 12 | 148639 | 49919969 | −0.4084 | 0.0621 | 2.0246 | 0.0633 | 2 | 0 |
| WCM172_X1_1 | 12 | 49920011 | 97301389 | 0.0658 | 1.0419 | 2.0843 | 2.0335 | 2 | 2 |
| WCM172_X1_1 | 12 | 97303569 | 101017445 | −0.3683 | 0.1604 | NA | NA | NA | NA |
| WCM172_X1_1 | 12 | 101017811 | 121758244 | 0.0856 | 1.0767 | 2.1092 | 2.1092 | 2 | 2 |
| WCM172_X1_1 | 12 | 122064924 | 133810926 | −0.3719 | 0.1515 | 2.1887 | 0.0328 | 2 | 0 |
| WCM172_X1_1 | 13 | 19600515 | 23914098 | −0.1726 | 0.5917 | 2.1796 | 0.8345 | 2 | 1 |
| WCM172_X1_1 | 13 | 23914443 | 86370473 | −0.4201 | 0.0327 | 2.0164 | 0.0295 | 2 | 0 |
| WCM172_X1_1 | 13 | 87045827 | 101944587 | 0.0796 | 1.0662 | 2.3828 | 1.8049 | 2 | 2 |
| WCM172_X1_1 | 13 | 101944688 | 115089411 | −0.3735 | 0.1479 | 2.0992 | 0.1166 | 2 | 0 |
| WCM172_X1_1 | 14 | 19377711 | 105207214 | 0.0363 | 0.9896 | 2.0212 | 1.9502 | 2 | 2 |
| WCM172_X1_1 | 14 | 105207489 | 107283019 | −0.3584 | 0.1841 | 2.1054 | 0.1667 | 2 | 0 |
| WCM172_X1_1 | 15 | 20170150 | 25326443 | −0.2904 | 0.3407 | 1.94 | 0.5927 | 2 | 1 |
| WCM172_X1_1 | 15 | 25327739 | 64792505 | −0.4126 | 0.0517 | 2.0611 | 0.0119 | 2 | 0 |
| WCM172_X1_1 | 15 | 64915075 | 67073865 | −0.171 | 0.595 | 1.8763 | 1.1446 | 2 | 1 |
| WCM172_X1_1 | 15 | 67212220 | 87097704 | −0.396 | 0.093 | 2.0373 | 0.0959 | 2 | 0 |
| WCM172_X1_1 | 15 | 87099493 | 102517646 | −0.1313 | 0.6747 | 2.0925 | 1.1001 | 2 | 1 |
| WCM172_X1_1 | 16 | 66966 | 30456006 | −0.0207 | 0.886 | 2.0972 | 1.599 | 2 | 2 |
| WCM172_X1_1 | 16 | 30456182 | 34578588 | 0.1732 | 1.2258 | 2.8892 | 1.7886 | 3 | 2 |
| WCM172_X1_1 | 16 | 34649016 | 49823410 | −0.0036 | 0.9174 | NA | NA | NA | NA |
| WCM172_X1_1 | 16 | 49856589 | 90128851 | −0.3898 | 0.1082 | 2.0765 | 0.0792 | 2 | 0 |
| WCM172_X1_1 | 16 | 90130117 | 90233299 | 0.2449 | 1.3436 | NA | NA | NA | NA |
| WCM172_X1_1 | 17 | 131595 | 2268602 | −0.4132 | 0.0502 | 2.1142 | −0.0434 | 2 | 0 |
| WCM172_X1_1 | 17 | 2270608 | 6558768 | 0.0307 | 0.9795 | 1.993 | 1.9506 | 2 | 2 |
| WCM172_X1_1 | 17 | 6589542 | 7960958 | −0.3996 | 0.084 | 1.9731 | 0.1468 | 2 | 0 |
| WCM172_X1_1 | 17 | 7976251 | 81083419 | 0.0578 | 1.0278 | 2.0677 | 2.01 | 2 | 2 |
| WCM172_X1_1 | 18 | 49030 | 78005215 | −0.0049 | 0.9151 | 1.9133 | 1.8581 | 2 | 2 |
| WCM172_X1_1 | 19 | 281467 | 18547530 | −0.4116 | 0.054 | 2.0479 | 0.0285 | 2 | 0 |
| WCM172_X1_1 | 19 | 18547805 | 31767965 | 0.0656 | 1.0416 | 2.2216 | 1.8955 | 2 | 2 |
| WCM172_X1_1 | 19 | 31768267 | 35504447 | −0.4326 | 9.00E−04 | 1.9239 | 0.0773 | 2 | 0 |
| WCM172_X1_1 | 19 | 35505284 | 37238741 | 0.1145 | 1.1265 | 2.3407 | 2.0259 | 2 | 2 |
| WCM172_X1_1 | 19 | 37239013 | 50753870 | −0.3945 | 0.0967 | 2.1385 | 2.00E−04 | 2 | 0 |
| WCM172_X1_1 | 19 | 50755952 | 59083970 | −0.1608 | 0.6158 | 2.0619 | 1.0029 | 2 | 1 |
| WCM172_X1_1 | 2 | 41620 | 145255317 | −0.1414 | 0.6547 | 2.0357 | 1.113 | 2 | 1 |
| WCM172_X1_1 | 2 | 145271267 | 152320118 | −0.5328 | −0.2714 | NA | NA | NA | NA |
| WCM172_X1_1 | 2 | 152320365 | 172725301 | −0.1744 | 0.5882 | 1.8684 | 1.1383 | 2 | 1 |
| WCM172_X1_1 | 2 | 172749765 | 175201998 | −0.7383 | −0.9688 | NA | NA | NA | NA |
| WCM172_X1_1 | 2 | 175213425 | 234926011 | −0.1418 | 0.6539 | 2.0471 | 1.0996 | 2 | 1 |
| WCM172_X1_1 | 2 | 234959482 | 240702118 | −0.6623 | −0.6824 | 1.2662 | −0.0199 | 1 | 0 |
| WCM172_X1_1 | 2 | 240721541 | 242573252 | −0.3606 | 0.1788 | 1.2074 | 1.0565 | 1 | 1 |
| WCM172_X1_1 | 2 | 242573369 | 242946607 | −0.7398 | −0.9749 | NA | NA | NA | NA |
| WCM172_X1_1 | 20 | 68406 | 590525 | −0.0363 | 0.8571 | 2.0308 | 1.592 | 2 | 2 |
| WCM172_X1_1 | 20 | 590855 | 35695515 | 0.0828 | 1.0716 | 2.1018 | 2.1018 | 2 | 2 |
| WCM172_X1_1 | 20 | 35696443 | 62904925 | −0.146 | 0.6456 | 2.1087 | 1.02 | 2 | 1 |
| WCM172_X1_1 | 21 | 9826235 | 48084282 | 0.0562 | 1.025 | 2.0997 | 1.9701 | 2 | 2 |
| WCM172_X1_1 | 22 | 16287461 | 50320941 | 0.051 | 1.0158 | 2.0614 | 1.9825 | 2 | 2 |
| WCM172_X1_1 | 22 | 50354843 | 51219138 | −0.4351 | −0.0054 | 1.941 | 0.0515 | 2 | 0 |
| WCM172_X1_1 | 3 | 239588 | 93780303 | 0.2115 | 1.2892 | 2.488 | 2.3998 | 2 | 2 |
| WCM172_X1_1 | 3 | 93781971 | 127806676 | −0.2456 | 0.4388 | 2.7073 | 0.0037 | 3 | 0 |
| WCM172_X1_1 | 3 | 127816309 | 168546914 | 0.1585 | 1.2012 | 2.3496 | 2.2491 | 2 | 2 |
| WCM172_X1_1 | 3 | 168802763 | 195802175 | 0.566 | 1.832 | 5.1028 | 2.0179 | 5 | 2 |
| WCM172_X1_1 | 3 | 195803944 | 197894713 | −0.4252 | 0.0197 | 1.8361 | 0.1914 | 2 | 0 |
| WCM172_X1_1 | 4 | 59394 | 344000 | −0.0537 | 0.8245 | NA | NA | NA | NA |
| WCM172_X1_1 | 4 | 366483 | 57891653 | 0.0616 | 1.0346 | 2.0666 | 2.0305 | 2 | 2 |
| WCM172_X1_1 | 4 | 57896500 | 190426502 | 0.2048 | 1.2782 | 2.7047 | 2.1462 | 3 | 2 |
| WCM172_X1_1 | 5 | 140636 | 54965423 | −0.1604 | 0.6165 | 2.0566 | 1.0097 | 2 | 1 |
| WCM172_X1_1 | 5 | 54965688 | 115833060 | −0.4195 | 0.0341 | 2.0359 | 0.012 | 2 | 0 |
| WCM172_X1_1 | 5 | 115838022 | 131728291 | 0.0723 | 1.0534 | 2.2355 | 1.9152 | 2 | 2 |
| WCM172_X1_1 | 5 | 131729504 | 132902891 | −0.3905 | 0.1064 | NA | NA | NA | NA |
| WCM172_X1_1 | 5 | 132918987 | 140085278 | −0.0241 | 0.8798 | 1.9661 | 1.7141 | 2 | 2 |
| WCM172_X1_1 | 5 | 140166824 | 143200053 | −0.3922 | 0.1023 | 2.0098 | 0.1371 | 2 | 0 |
| WCM172_X1_1 | 5 | 143538043 | 169446096 | 0.0776 | 1.0626 | 2.1435 | 2.0339 | 2 | 2 |
| WCM172_X1_1 | 5 | 169454923 | 174869239 | −0.4176 | 0.0391 | NA | NA | NA | NA |
| WCM172_X1_1 | 5 | 174869327 | 180687773 | 0.0828 | 1.0717 | 2.201 | 2.003 | 2 | 2 |
| WCM172_X1_1 | 6 | 203509 | 13807190 | 0.0101 | 0.9425 | 2.0299 | 1.8138 | 2 | 2 |
| WCM172_X1_1 | 6 | 13814338 | 22570064 | −0.4076 | 0.0641 | 1.9066 | 0.1843 | 2 | 0 |
| WCM172_X1_1 | 6 | 22570479 | 80982916 | 0.0314 | 0.9808 | 2.009 | 1.9382 | 2 | 2 |
| WCM172_X1_1 | 6 | 81053489 | 153347598 | −0.4138 | 0.0486 | 2.0164 | 0.0521 | 2 | 0 |
| WCM172_X1_1 | 6 | 153365130 | 170893411 | −0.1335 | 0.6704 | 2.0775 | 1.1056 | 2 | 1 |
| WCM172_X1_1 | 7 | 193253 | 158937400 | 0.0487 | 1.0117 | 2.0522 | 1.9805 | 2 | 2 |
| WCM172_X1_1 | 8 | 190906 | 17539139 | −0.4037 | 0.0738 | 2.0521 | 0.053 | 2 | 0 |
| WCM172_X1_1 | 8 | 17541999 | 30209791 | −0.6722 | −0.7173 | 1.0753 | 0.1412 | 1 | 0 |
| WCM172_X1_1 | 8 | 30210180 | 47752745 | −0.4327 | 7.00E−04 | 2.0545 | −0.0536 | 2 | 0 |
| WCM172_X1_1 | 8 | 47753945 | 146279424 | 1.1134 | 2.5668 | 9.9019 | 1.9483 | 10 | 2 |
| WCM172_X1_1 | 9 | 17086 | 5466769 | −0.4063 | 0.0674 | 1.9202 | 0.1754 | 2 | 0 |
| WCM172_X1_1 | 9 | 5467856 | 19328063 | 0.0566 | 1.0258 | 3.9837 | 0.0884 | 4 | 0 |
| WCM172_X1_1 | 9 | 19332036 | 41954826 | 0.4354 | 1.6401 | 3.9968 | 2.2368 | 4 | 2 |
| WCM172_X1_1 | 9 | 43624879 | 103091424 | 0.0619 | 1.0351 | 2.0854 | 2.0131 | 2 | 2 |
| WCM172_X1_1 | 9 | 103092362 | 105765508 | −0.4457 | −0.033 | NA | NA | NA | NA |
| WCM172_X1_1 | 9 | 105767036 | 117064377 | 0.0337 | 0.9849 | 2.0855 | 1.8729 | 2 | 2 |
| WCM172_X1_1 | 9 | 117066944 | 125143995 | −0.4244 | 0.0219 | 1.9129 | 0.1176 | 2 | 0 |
| WCM172_X1_1 | 9 | 125145982 | 126439014 | 0.0273 | 0.9735 | 1.9636 | 1.9636 | 2 | 2 |
| WCM172_X1_1 | 9 | 126520068 | 130331147 | −0.432 | 0.0025 | 1.8305 | 0.173 | 2 | 0 |
| WCM172_X1_1 | 9 | 130341207 | 141111414 | 0.0686 | 1.0468 | 2.095 | 2.037 | 2 | 2 |
| WCM172_X1_1 | X | 2700141 | 37701149 | −0.3679 | 0.1613 | NA | NA | NA | NA |
| WCM172_X1_1 | X | 37705579 | 65420492 | 0.056 | 1.0246 | NA | NA | NA | NA |
| WCM172_X1_1 | X | 65423237 | 154509348 | 0.3854 | 1.5643 | NA | NA | NA | NA |
| WCM172_X2_1 | 1 | 721476 | 979270 | −0.1113 | 0.5712 | NA | NA | NA | NA |
| WCM172_X2_1 | 1 | 979736 | 11007907 | −0.0625 | 0.6281 | 1.9764 | 1.1146 | 2 | 1 |
| WCM172_X2_1 | 1 | 11008238 | 43300805 | −0.2373 | 0.423 | 1.7511 | 0.9303 | 2 | 1 |
| WCM172_X2_1 | 1 | 43304619 | 160971139 | −0.1006 | 0.5838 | 1.9482 | 1.0493 | 2 | 1 |
| WCM172_X2_1 | 1 | 160990852 | 178514702 | −0.5577 | 0.034 | 1.978 | 0.0697 | 2 | 0 |
| WCM172_X2_1 | 1 | 178514941 | 200842232 | 0.058 | 0.7672 | 3.3825 | 0.0213 | 3 | 0 |
| WCM172_X2_1 | 1 | 200842459 | 249212550 | −0.1301 | 0.5492 | 2.8682 | 0.0584 | 3 | 0 |
| WCM172_X2_1 | 10 | 255953 | 135440117 | −0.2418 | 0.4175 | 1.74 | 0.9313 | 2 | 1 |
| WCM172_X2_1 | 11 | 193112 | 79133220 | −0.0106 | 0.6882 | 1.7342 | 1.4885 | 2 | 1 |
| WCM172_X2_1 | 11 | 81601786 | 84245693 | −0.5804 | 0.0056 | NA | NA | NA | NA |
| WCM172_X2_1 | 11 | 84634139 | 134856574 | 0.0131 | 0.7156 | 1.68 | 1.6043 | 2 | 2 |
| WCM172_X2_1 | 12 | 148639 | 33532891 | −0.445 | 0.1729 | 2.1948 | 0.0599 | 2 | 0 |
| WCM172_X2_1 | 12 | 33538123 | 49724853 | −0.6873 | −0.1291 | 1.7784 | 0.0504 | 2 | 0 |
| WCM172_X2_1 | 12 | 49725091 | 49906650 | −0.4038 | 0.2231 | NA | NA | NA | NA |
| WCM172_X2_1 | 12 | 49908354 | 97254620 | 0.1231 | 0.8416 | 1.8165 | 1.7675 | 2 | 2 |
| WCM172_X2_1 | 12 | 97301389 | 100955712 | −0.6313 | −0.0581 | NA | NA | NA | NA |
| WCM172_X2_1 | 12 | 100957179 | 121758192 | 0.0047 | 0.7058 | 1.6593 | 1.6028 | 2 | 2 |
| WCM172_X2_1 | 12 | 122064924 | 133810926 | −0.627 | −0.0527 | 1.8772 | 0.0511 | 2 | 0 |
| WCM172_X2_1 | 13 | 19600515 | 24243208 | −0.247 | 0.4114 | 1.7407 | 0.9192 | 2 | 1 |
| WCM172_X2_1 | 13 | 24304501 | 88327790 | −0.782 | −0.2509 | 1.6429 | 0.0379 | 2 | 0 |
| WCM172_X2_1 | 13 | 88327974 | 101881814 | 0.18 | 0.9062 | 1.9506 | 1.7976 | 2 | 2 |
| WCM172_X2_1 | 13 | 101910874 | 115089411 | −0.6928 | −0.1361 | 1.715 | 0.1049 | 2 | 0 |
| WCM172_X2_1 | 14 | 19377697 | 105181089 | 0.1153 | 0.8327 | 1.8074 | 1.7546 | 2 | 2 |
| WCM172_X2_1 | 14 | 105181673 | 107283203 | −0.4558 | 0.1597 | 2.1492 | 0.0849 | 2 | 0 |
| WCM172_X2_1 | 15 | 20170150 | 25325366 | −0.3983 | 0.2298 | 1.7346 | 0.6107 | 2 | 1 |
| WCM172_X2_1 | 15 | 25326470 | 64792592 | −0.5575 | 0.0343 | 1.9464 | 0.1016 | 2 | 0 |
| WCM172_X2_1 | 15 | 64915214 | 65944142 | −0.0862 | 0.6005 | NA | NA | NA | NA |
| WCM172_X2_1 | 15 | 65945098 | 86838568 | −0.2098 | 0.4555 | 1.9598 | 0.7827 | 2 | 1 |
| WCM172_X2_1 | 15 | 86940607 | 102517641 | −0.0914 | 0.5945 | 1.9816 | 1.0383 | 2 | 1 |
| WCM172_X2_1 | 16 | 66953 | 30424927 | 0.2145 | 0.9453 | 1.9647 | 1.8864 | 2 | 2 |
| WCM172_X2_1 | 16 | 30429046 | 46727007 | 0.4887 | 1.2515 | 2.8413 | 1.9205 | 3 | 2 |
| WCM172_X2_1 | 16 | 46729489 | 49764837 | 0.2435 | 0.9781 | NA | NA | NA | NA |
| WCM172_X2_1 | 16 | 49823411 | 90233299 | −0.5257 | 0.0737 | 2.0215 | 0.0834 | 2 | 0 |
| WCM172_X2_1 | 17 | 131595 | 2268617 | −0.7419 | −0.199 | 1.6716 | 0.0706 | 2 | 0 |
| WCM172_X2_1 | 17 | 2270608 | 6558768 | 0.0395 | 0.7459 | 1.6982 | 1.6558 | 2 | 2 |
| WCM172_X2_1 | 17 | 6589642 | 7960983 | −0.6051 | −0.0252 | 1.837 | 0.1284 | 2 | 0 |
| WCM172_X2_1 | 17 | 7976140 | 19871755 | 0.2626 | 0.9995 | 1.9993 | 1.9993 | 2 | 2 |
| WCM172_X2_1 | 17 | 20000049 | 21202191 | −0.4132 | 0.2117 | 1.5452 | 0.7708 | 2 | 1 |
| WCM172_X2_1 | 17 | 21203893 | 81083455 | 0.2573 | 0.9935 | 2.0067 | 1.9753 | 2 | 2 |
| WCM172_X2_1 | 18 | 49022 | 78005224 | 0.1755 | 0.9011 | 1.8823 | 1.8527 | 2 | 2 |
| WCM172_X2_1 | 19 | 281499 | 18547178 | −0.4497 | 0.1672 | 2.1621 | 0.0836 | 2 | 0 |
| WCM172_X2_1 | 19 | 18547507 | 31048071 | 0.1546 | 0.8775 | 2.0125 | 1.6618 | 2 | 2 |
| WCM172_X2_1 | 19 | 31767480 | 35502443 | −0.4925 | 0.1147 | 2.081 | 0.0845 | 2 | 0 |
| WCM172_X2_1 | 19 | 35504167 | 37211397 | 0.2215 | 0.9532 | 2.0389 | 1.8334 | 2 | 2 |
| WCM172_X2_1 | 19 | 37211517 | 50655886 | −0.4134 | 0.2115 | 2.2306 | 0.0851 | 2 | 0 |
| WCM172_X2_1 | 19 | 50657885 | 59083988 | 0.0747 | 0.7863 | 2.404 | 1.0454 | 2 | 1 |
| WCM172_X2_1 | 2 | 41609 | 100360602 | −0.232 | 0.4292 | 1.6275 | 1.0655 | 2 | 1 |
| WCM172_X2_1 | 2 | 100368748 | 103335652 | 0.2772 | 1.016 | 2.9357 | 1.1088 | 3 | 1 |
| WCM172_X2_1 | 2 | 103340208 | 111408315 | −0.0542 | 0.6377 | NA | NA | NA | NA |
| WCM172_X2_1 | 2 | 111411035 | 145182370 | −0.5251 | 0.0745 | 1.0764 | 1.0295 | 1 | 1 |
| WCM172_X2_1 | 2 | 145187370 | 151343916 | −1.1317 | −0.7234 | NA | NA | NA | NA |
| WCM172_X2_1 | 2 | 151857850 | 172666765 | −0.1526 | 0.5228 | 1.8348 | 1.0387 | 2 | 1 |
| WCM172_X2_1 | 2 | 172669928 | 175202000 | −1.1904 | −0.8069 | NA | NA | NA | NA |
| WCM172_X2_1 | 2 | 175213297 | 184022202 | −0.1237 | 0.5567 | 1.9149 | 1.0269 | 2 | 1 |
| WCM172_X2_1 | 2 | 184023082 | 234925980 | 0.2694 | 1.0072 | 2.0326 | 1.9874 | 2 | 2 |
| WCM172_X2_1 | 2 | 234959435 | 240834686 | −0.4714 | 0.1406 | 2.2126 | −0.0079 | 2 | 0 |
| WCM172_X2_1 | 2 | 240882476 | 242572846 | −0.0308 | 0.6648 | 2.0626 | 1.1082 | 2 | 1 |
| WCM172_X2_1 | 2 | 242573114 | 242946616 | −0.427 | 0.1949 | NA | NA | NA | NA |
| WCM172_X2_1 | 20 | 68363 | 62904881 | 0.2375 | 0.9712 | 1.9605 | 1.9605 | 2 | 2 |
| WCM172_X2_1 | 21 | 9826207 | 48084226 | 0.3405 | 1.0869 | 2.1858 | 2.0624 | 2 | 2 |
| WCM172_X2_1 | 22 | 16287437 | 50320941 | −0.0598 | 0.6312 | 1.8996 | 1.1981 | 2 | 1 |
| WCM172_X2_1 | 22 | 50355176 | 51215124 | −0.9657 | −0.494 | 1.2938 | 0.1264 | 1 | 0 |
| WCM172_X2_1 | 3 | 239350 | 93629510 | 0.358 | 1.1064 | 2.6409 | 1.6653 | 3 | 2 |
| WCM172_X2_1 | 3 | 93643108 | 127788325 | −0.221 | 0.4423 | 2.6522 | 0.0653 | 3 | 0 |
| WCM172_X2_1 | 3 | 127800229 | 168542702 | 0.3823 | 1.1335 | 2.5556 | 1.8322 | 3 | 2 |
| WCM172_X2_1 | 3 | 168546914 | 195956827 | 0.9638 | 1.7685 | 5.0381 | 1.776 | 5 | 2 |
| WCM172_X2_1 | 3 | 195959323 | 197894719 | −0.7303 | −0.1841 | 1.66 | 0.1004 | 2 | 0 |
| WCM172_X2_1 | 4 | 59394 | 17636697 | 0.1995 | 0.9283 | 1.9397 | 1.8665 | 2 | 2 |
| WCM172_X2_1 | 4 | 17638206 | 74027040 | 0.0608 | 0.7704 | 1.7273 | 1.6842 | 2 | 2 |
| WCM172_X2_1 | 4 | 74043189 | 190426502 | 0.1884 | 0.9158 | 2.1194 | 1.6537 | 2 | 2 |
| WCM172_X2_1 | 5 | 140532 | 55034800 | −0.239 | 0.4209 | 1.7105 | 0.967 | 2 | 1 |
| WCM172_X2_1 | 5 | 55038000 | 115833029 | −0.706 | −0.1529 | 1.727 | 0.0718 | 2 | 0 |
| WCM172_X2_1 | 5 | 115837962 | 131726445 | 0.0803 | 0.7927 | 1.9285 | 1.536 | 2 | 2 |
| WCM172_X2_1 | 5 | 131728263 | 132939664 | −0.758 | −0.2197 | NA | NA | NA | NA |
| WCM172_X2_1 | 5 | 133292650 | 134989582 | 0.0467 | 0.7542 | NA | NA | NA | NA |
| WCM172_X2_1 | 5 | 135170558 | 137476428 | −0.6076 | −0.0284 | 1.8702 | 0.0909 | 2 | 0 |
| WCM172_X2_1 | 5 | 137481513 | 140085334 | −0.0021 | 0.698 | 1.6916 | 1.553 | 2 | 2 |
| WCM172_X2_1 | 5 | 140166824 | 143200053 | −0.8144 | −0.293 | 1.569 | 0.0635 | 2 | 0 |
| WCM172_X2_1 | 5 | 143538036 | 169361700 | 0.1107 | 0.8275 | 1.7908 | 1.7584 | 2 | 2 |
| WCM172_X2_1 | 5 | 169410122 | 174178761 | −0.6421 | −0.0717 | NA | NA | NA | NA |
| WCM172_X2_1 | 5 | 174869014 | 180687776 | 0.1277 | 0.8468 | 1.9828 | 1.6141 | 2 | 2 |
| WCM172_X2_1 | 6 | 203515 | 407514 | −0.1474 | 0.529 | NA | NA | NA | NA |
| WCM172_X2_1 | 6 | 486679 | 13807193 | −0.2863 | 0.3646 | 1.7103 | 0.8647 | 2 | 1 |
| WCM172_X2_1 | 6 | 13814338 | 22570496 | −0.6925 | −0.1357 | 1.633 | 0.1874 | 2 | 0 |
| WCM172_X2_1 | 6 | 24134663 | 82462292 | 0.0068 | 0.7083 | 1.7638 | 1.5038 | 2 | 2 |
| WCM172_X2_1 | 6 | 82881145 | 153344531 | −0.7015 | −0.1472 | 1.7355 | 0.0705 | 2 | 0 |
| WCM172_X2_1 | 6 | 153345426 | 170893438 | −0.2172 | 0.4467 | 1.7322 | 0.9935 | 2 | 1 |
| WCM172_X2_1 | 7 | 193451 | 158937416 | 0.0746 | 0.7862 | 1.7423 | 1.7068 | 2 | 2 |
| WCM172_X2_1 | 8 | 190900 | 36662784 | −0.989 | −0.5255 | 1.3343 | 0.0551 | 1 | 0 |
| WCM172_X2_1 | 8 | 36663789 | 43212034 | −0.5397 | 0.0564 | 2.0137 | 0.066 | 2 | 0 |
| WCM172_X2_1 | 8 | 47752739 | 146279476 | 1.4512 | 2.286 | 8.0404 | 1.7137 | 8 | 2 |
| WCM172_X2_1 | 9 | 17056 | 5774170 | −0.7451 | −0.2031 | 1.6551 | 0.0822 | 2 | 0 |
| WCM172_X2_1 | 9 | 5774225 | 19296139 | 0.1741 | 0.8995 | 3.6784 | 0.0525 | 4 | 0 |
| WCM172_X2_1 | 9 | 19298087 | 71395477 | 0.6267 | 1.4033 | 3.6901 | 1.5999 | 4 | 2 |
| WCM172_X2_1 | 9 | 71395635 | 71606148 | 0.1021 | 0.8176 | NA | NA | NA | NA |
| WCM172_X2_1 | 9 | 71623366 | 103066014 | 0.0596 | 0.769 | 1.7353 | 1.6728 | 2 | 2 |
| WCM172_X2_1 | 9 | 103070951 | 106858549 | −0.7397 | −0.1961 | NA | NA | NA | NA |
| WCM172_X2_1 | 9 | 106860755 | 117064359 | 0.005 | 0.7062 | 1.6616 | 1.6014 | 2 | 2 |
| WCM172_X2_1 | 9 | 117066929 | 125273843 | −0.7623 | −0.2253 | 1.6248 | 0.086 | 2 | 0 |
| WCM172_X2_1 | 9 | 125281589 | 126319974 | 0.042 | 0.7488 | 1.7518 | 1.6089 | 2 | 2 |
| WCM172_X2_1 | 9 | 126345454 | 130453061 | −0.7801 | −0.2483 | 1.6815 | 0.0022 | 2 | 0 |
| WCM172_X2_1 | 9 | 130455534 | 141111414 | 0.0481 | 0.7558 | 1.7273 | 1.6498 | 2 | 2 |
| WCM172_X2_1 | X | 2700119 | 47486962 | −0.2064 | 0.4595 | NA | NA | NA | NA |
| WCM172_X2_1 | X | 47487606 | 154509348 | 0.5056 | 1.2702 | NA | NA | NA | NA |
| WCM176 | 1 | 14907 | 249213973 | 0.0562 | NA | NA | NA | NA | NA |
| WCM176 | 10 | 226083 | 135438991 | 0.0511 | NA | NA | NA | NA | NA |
| WCM176 | 11 | 193112 | 134320516 | 0.0406 | NA | NA | NA | NA | NA |
| WCM176 | 12 | 148465 | 133810703 | 0.0319 | NA | NA | NA | NA | NA |
| WCM176 | 13 | 19042005 | 115089411 | 0.0251 | NA | NA | NA | NA | NA |
| WCM176 | 14 | 19110349 | 107283270 | 0.034 | NA | NA | NA | NA | NA |
| WCM176 | 15 | 20083896 | 102514170 | 0.0522 | NA | NA | NA | NA | NA |
| WCM176 | 16 | 66882 | 90234899 | 0.0223 | NA | NA | NA | NA | NA |
| WCM176 | 17 | 131595 | 81187855 | 0.0303 | NA | NA | NA | NA | NA |
| WCM176 | 18 | 47708 | 78005274 | 0.0307 | NA | NA | NA | NA | NA |
| WCM176 | 19 | 105021 | 59093696 | −0.0308 | NA | NA | NA | NA | NA |
| WCM176 | 2 | 41620 | 243037022 | 0.0361 | NA | NA | NA | NA | NA |
| WCM176 | 20 | 68340 | 62934592 | 0.0624 | NA | NA | NA | NA | NA |
| WCM176 | 21 | 9590314 | 48084198 | 0.0248 | NA | NA | NA | NA | NA |
| WCM176 | 22 | 16159060 | 51216336 | 0.0936 | NA | NA | NA | NA | NA |
| WCM176 | 3 | 239349 | 197896724 | 0.0357 | NA | NA | NA | NA | NA |
| WCM176 | 4 | 53435 | 190903980 | 0.0332 | NA | NA | NA | NA | NA |
| WCM176 | 5 | 90750 | 180687821 | 0.0266 | NA | NA | NA | NA | NA |
| WCM176 | 6 | 203466 | 170892873 | 0.0307 | NA | NA | NA | NA | NA |
| WCM176 | 7 | 193245 | 158937158 | 0.0292 | NA | NA | NA | NA | NA |
| WCM176 | 8 | 163457 | 146279390 | 0.0191 | NA | NA | NA | NA | NA |
| WCM176 | 9 | 116800 | 141107536 | 0.0557 | NA | NA | NA | NA | NA |
| WCM176 | X | 2700125 | 154493609 | −0.2993 | NA | NA | NA | NA | NA |
| WCM176_Z1_1 | 1 | 69610 | 82459717 | 0.0418 | −0.011 | 1.0031 | 0.9816 | 1 | 1 |
| WCM176_Z1_1 | 1 | 84335658 | 107599765 | −0.6887 | −0.9526 | 1.0398 | −0.0064 | 1 | 0 |
| WCM176_Z1_1 | 1 | 107599970 | 249212354 | 0.0523 | 0.0015 | 1.0107 | 0.9914 | 1 | 1 |
| WCM176_Z1_1 | 10 | 225995 | 87628923 | 0.0686 | 0.0211 | 1.0245 | 1.005 | 1 | 1 |
| WCM176_Z1_1 | 10 | 87898622 | 89269470 | −0.6543 | −0.9047 | NA | NA | NA | NA |
| WCM176_Z1_1 | 10 | 89280886 | 90122349 | −2.3104 | −5.0762 | NA | NA | NA | NA |
| WCM176_Z1_1 | 10 | 90332716 | 96313960 | −0.6931 | −0.9587 | 0.9907 | 0.0383 | 1 | 0 |
| WCM176_Z1_1 | 10 | 96316943 | 135381727 | 0.0687 | 0.0213 | 1.0405 | 0.9893 | 1 | 1 |
| WCM176_Z1_1 | 11 | 193112 | 19261653 | 0.0627 | 0.0141 | 1.022 | 0.9977 | 1 | 1 |
| WCM176_Z1_1 | 11 | 19372553 | 20419168 | −0.7091 | −0.9812 | NA | NA | NA | NA |
| WCM176_Z1_1 | 11 | 20424525 | 106810318 | 0.063 | 0.0145 | 1.0247 | 0.9955 | 1 | 1 |
| WCM176_Z1_1 | 11 | 106810789 | 122968641 | −0.6814 | −0.9423 | 1.0396 | 0.0011 | 1 | 0 |
| WCM176_Z1_1 | 11 | 123065632 | 134856534 | 0.0722 | 0.0255 | 1.0288 | 1.0068 | 1 | 1 |
| WCM176_Z1_1 | 12 | 148637 | 12790582 | 0.0826 | 0.038 | 1.034 | 1.0193 | 1 | 1 |
| WCM176_Z1_1 | 12 | 12794929 | 14720583 | −0.723 | −1.0007 | 0.9493 | 0.0502 | 1 | 0 |
| WCM176_Z1_1 | 12 | 14766139 | 85623336 | 0.0716 | 0.0248 | 1.0308 | 1.0039 | 1 | 1 |
| WCM176_Z1_1 | 12 | 85674163 | 88568412 | −0.7308 | −1.0117 | NA | NA | NA | NA |
| WCM176_Z1_1 | 12 | 88582704 | 133810703 | 0.0686 | 0.0212 | 1.0245 | 1.005 | 1 | 1 |
| WCM176_Z1_1 | 13 | 19600159 | 40362474 | 0.0775 | 0.0319 | 1.0493 | 0.9953 | 1 | 1 |
| WCM176_Z1_1 | 13 | 41133697 | 53286967 | −0.7151 | −0.9896 | 0.996 | 0.0113 | 1 | 0 |
| WCM176_Z1_1 | 13 | 53307501 | 53624856 | −2.2239 | −4.5023 | NA | NA | NA | NA |
| WCM176_Z1_1 | 13 | 54886157 | 66879145 | −0.7312 | −1.0123 | NA | NA | NA | NA |
| WCM176_Z1_1 | 13 | 67205392 | 67802328 | −2.2171 | −4.4645 | NA | NA | NA | NA |
| WCM176_Z1_1 | 13 | 70281805 | 79175910 | −0.7027 | −0.9722 | 1.0371 | −0.0176 | 1 | 0 |
| WCM176_Z1_1 | 13 | 79176545 | 115089411 | 0.0056 | −0.0549 | 0.9883 | 0.937 | 1 | 1 |
| WCM176_Z1_1 | 14 | 19377723 | 107283203 | 0.0481 | −0.0035 | 1.0072 | 0.988 | 1 | 1 |
| WCM176_Z1_1 | 15 | 20170011 | 102515303 | 0.0267 | −0.0294 | 0.9881 | 0.9716 | 1 | 1 |
| WCM176_Z1_1 | 16 | 67604 | 90233622 | 0.0209 | −0.0363 | 0.9846 | 0.9657 | 1 | 1 |
| WCM176_Z1_1 | 17 | 131595 | 81083567 | 0.0169 | −0.0412 | 0.9718 | 0.9718 | 1 | 1 |
| WCM176_Z1_1 | 18 | 48248 | 37202112 | 0.0354 | −0.0189 | 1.0013 | 0.9727 | 1 | 1 |
| WCM176_Z1_1 | 18 | 39535310 | 60795987 | −0.7315 | −1.0127 | 0.9971 | −0.0059 | 1 | 0 |
| WCM176_Z1_1 | 18 | 60985357 | 67871369 | −0.0167 | −0.082 | 0.9838 | 0.9057 | 1 | 1 |
| WCM176_Z1_1 | 18 | 67872491 | 72353065 | −0.6893 | −0.9534 | NA | NA | NA | NA |
| WCM176_Z1_1 | 18 | 72493448 | 78005210 | −0.0476 | −0.1197 | 0.9913 | 0.8495 | 1 | 1 |
| WCM176_Z1_1 | 19 | 281435 | 59083923 | 0.0289 | −0.0266 | 0.9912 | 0.9722 | 1 | 1 |
| WCM176_Z1_1 | 2 | 41612 | 101656726 | 0.0202 | −0.0372 | 0.9828 | 0.9663 | 1 | 1 |
| WCM176_Z1_1 | 2 | 101666894 | 106509634 | −0.7521 | −1.0419 | 0.9262 | 0.0451 | 1 | 0 |
| WCM176_Z1_1 | 2 | 106687385 | 138771622 | 0.0542 | 0.0038 | 1.0099 | 0.9954 | 1 | 1 |
| WCM176_Z1_1 | 2 | 139045855 | 151890398 | −0.7062 | −0.9771 | NA | NA | NA | NA |
| WCM176_Z1_1 | 2 | 151900286 | 159434699 | 0.0223 | −0.0347 | 1.0081 | 0.9443 | 1 | 1 |
| WCM176_Z1_1 | 2 | 159477620 | 165600288 | −0.6667 | −0.9219 | 1.0304 | 0.0252 | 1 | 0 |
| WCM176_Z1_1 | 2 | 165630226 | 176046091 | 0.0562 | 0.0063 | 1.0152 | 0.9935 | 1 | 1 |
| WCM176_Z1_1 | 2 | 176794846 | 177202287 | −0.8142 | −1.131 | NA | NA | NA | NA |
| WCM176_Z1_1 | 2 | 178077593 | 242946616 | 0.0455 | −0.0067 | 1.005 | 0.9858 | 1 | 1 |
| WCM176_Z1_1 | 20 | 68363 | 62904932 | 0.0301 | −0.0253 | 0.9909 | 0.9743 | 1 | 1 |
| WCM176_Z1_1 | 21 | 9907310 | 48084277 | 0.0325 | −0.0223 | 1.0037 | 0.9656 | 1 | 1 |
| WCM176_Z1_1 | 22 | 17063476 | 51215157 | 0.0191 | −0.0385 | 0.9866 | 0.9607 | 1 | 1 |
| WCM176_Z1_1 | 3 | 239524 | 197894719 | 0.0385 | −0.0151 | 0.9991 | 0.98 | 1 | 1 |
| WCM176_Z1_1 | 4 | 59331 | 15018876 | 0.0641 | 0.0158 | 1.0329 | 0.9891 | 1 | 1 |
| WCM176_Z1_1 | 4 | 15042102 | 44724171 | −0.6679 | −0.9236 | 1.0425 | 0.0119 | 1 | 0 |
| WCM176_Z1_1 | 4 | 46053591 | 105393505 | 0.0613 | 0.0124 | 1.0207 | 0.9965 | 1 | 1 |
| WCM176_Z1_1 | 4 | 105412061 | 107845325 | −0.6217 | −0.8598 | NA | NA | NA | NA |
| WCM176_Z1_1 | 4 | 107845722 | 118006173 | 0.0622 | 0.0135 | 1.0689 | 0.9499 | 1 | 1 |
| WCM176_Z1_1 | 4 | 118006317 | 138453077 | −0.6272 | −0.8674 | 1.085 | 0.0113 | 1 | 0 |
| WCM176_Z1_1 | 4 | 139100434 | 175252660 | 0.0651 | 0.017 | 1.0325 | 0.9912 | 1 | 1 |
| WCM176_Z1_1 | 4 | 175344986 | 178897031 | −0.6948 | −0.9612 | 1.0511 | −0.0238 | 1 | 0 |
| WCM176_Z1_1 | 4 | 178911657 | 190426502 | 0.0624 | 0.0138 | 1.0679 | 0.9513 | 1 | 1 |
| WCM176_Z1_1 | 5 | 140448 | 2753409 | 0.042 | −0.0109 | 1.0487 | 0.9362 | 1 | 1 |
| WCM176_Z1_1 | 5 | 2755166 | 5489412 | −0.7325 | −1.0142 | NA | NA | NA | NA |
| WCM176_Z1_1 | 5 | 6312525 | 64595822 | 0.0725 | 0.0259 | 1.0364 | 0.9998 | 1 | 1 |
| WCM176_Z1_1 | 5 | 64625302 | 81614091 | −0.6886 | −0.9525 | 1.0204 | 0.0131 | 1 | 0 |
| WCM176_Z1_1 | 5 | 82352930 | 98115828 | 0.0645 | 0.0163 | 1.0503 | 0.9724 | 1 | 1 |
| WCM176_Z1_1 | 5 | 98128892 | 98240642 | −1.9783 | −3.4686 | NA | NA | NA | NA |
| WCM176_Z1_1 | 5 | 99715473 | 101834445 | −0.6751 | −0.9336 | NA | NA | NA | NA |
| WCM176_Z1_1 | 5 | 102201966 | 180687814 | 0.0764 | 0.0305 | 1.0324 | 1.0103 | 1 | 1 |
| WCM176_Z1_1 | 6 | 203708 | 6649864 | 0.0404 | −0.0128 | 1.0594 | 0.923 | 1 | 1 |
| WCM176_Z1_1 | 6 | 6654856 | 9977777 | −0.7155 | −0.9901 | 1.0113 | −0.0044 | 1 | 0 |
| WCM176_Z1_1 | 6 | 10393779 | 73814985 | 0.0504 | −7.00E−04 | 1.0091 | 0.9899 | 1 | 1 |
| WCM176_Z1_1 | 6 | 73821042 | 127607935 | −0.6852 | −0.9477 | 1.0377 | −9.00E−04 | 1 | 0 |
| WCM176_Z1_1 | 6 | 127608082 | 129649548 | 0.0582 | 0.0087 | NA | NA | NA | NA |
| WCM176_Z1_1 | 6 | 129670483 | 129764207 | −0.9363 | −1.3115 | NA | NA | NA | NA |
| WCM176_Z1_1 | 6 | 129766885 | 170893435 | 0.0643 | 0.016 | 1.0403 | 0.982 | 1 | 1 |
| WCM176_Z1_1 | 7 | 193253 | 48964707 | 0.0505 | −6.00E−04 | 1.0152 | 0.9839 | 1 | 1 |
| WCM176_Z1_1 | 7 | 48965266 | 53104078 | −0.6601 | −0.9129 | 1.0934 | −0.0311 | 1 | 0 |
| WCM176_Z1_1 | 7 | 54612356 | 158937400 | 0.0678 | 0.0202 | 1.036 | 0.9921 | 1 | 1 |
| WCM176_Z1_1 | 8 | 190906 | 146279475 | 0.056 | 0.006 | 1.015 | 0.9933 | 1 | 1 |
| WCM176_Z1_1 | 9 | 116832 | 6015279 | 0.0561 | 0.0061 | NA | NA | NA | NA |
| WCM176_Z1_1 | 9 | 6241712 | 21481451 | −0.6786 | −0.9385 | 1.0427 | 8.00E−04 | 1 | 0 |
| WCM176_Z1_1 | 9 | 21512126 | 24545918 | −2.0474 | −3.7078 | NA | NA | NA | NA |
| WCM176_Z1_1 | 9 | 25677782 | 27949335 | −0.6723 | −0.9297 | NA | NA | NA | NA |
| WCM176_Z1_1 | 9 | 27949506 | 141111414 | 0.0558 | 0.0058 | 1.0149 | 0.9932 | 1 | 1 |
| WCM176_Z1_1 | X | 2712633 | 154509348 | 0.0187 | −0.039 | NA | NA | NA | NA |
| WCM183 | 1 | 14930 | 16865953 | −0.4962 | 0.0638 | 2.0368 | 0.0537 | 2 | 0 |
| WCM183 | 1 | 16890598 | 17250991 | −0.7439 | −0.3517 | 1.2724 | 0.2948 | 1 | 0 |
| WCM183 | 1 | 17256433 | 22850960 | −0.4559 | 0.1271 | 2.1134 | 0.0708 | 2 | 0 |
| WCM183 | 1 | 22852796 | 65068581 | −0.1514 | 0.578 | 1.8971 | 1.0885 | 2 | 1 |
| WCM183 | 1 | 65095054 | 75055696 | −0.4971 | 0.0623 | 1.0441 | 1.0441 | 1 | 1 |
| WCM183 | 1 | 75065485 | 85511022 | −0.1534 | 0.5752 | 1.8431 | 1.1366 | 2 | 1 |
| WCM183 | 1 | 85537522 | 91967454 | −0.5293 | 0.0109 | 1.0076 | 1.0076 | 1 | 1 |
| WCM183 | 1 | 91973516 | 115537286 | −0.1765 | 0.5425 | 1.8497 | 1.0633 | 2 | 1 |
| WCM183 | 1 | 115537869 | 151553583 | 0.0901 | 0.908 | 1.884 | 1.869 | 2 | 2 |
| WCM183 | 1 | 151554137 | 154926249 | 0.4047 | 1.3121 | 2.5791 | 2.3871 | 3 | 2 |
| WCM183 | 1 | 154930410 | 155451162 | 0.2339 | 1.0959 | 2.2283 | 2.0468 | 2 | 2 |
| WCM183 | 1 | 155451288 | 158450576 | −0.4608 | 0.1195 | 2.0957 | 0.077 | 2 | 0 |
| WCM183 | 1 | 158507184 | 212002590 | 0.1852 | 1.0329 | 2.0461 | 2.0461 | 2 | 2 |
| WCM183 | 1 | 212002684 | 215751313 | −0.4982 | 0.0606 | 2.0993 | −0.0135 | 2 | 0 |
| WCM183 | 1 | 215752370 | 249214018 | 0.1508 | 0.9881 | 1.9836 | 1.9836 | 2 | 2 |
| WCM183 | 10 | 225938 | 30750519 | −0.5388 | −0.0044 | 1.9564 | 0.0375 | 2 | 0 |
| WCM183 | 10 | 30807392 | 39011152 | 0.124 | 0.9529 | 1.9892 | 1.8822 | 2 | 2 |
| WCM183 | 10 | 42681235 | 52603945 | −0.4294 | 0.1683 | 2.1692 | 0.0783 | 2 | 0 |
| WCM183 | 10 | 52610365 | 135477898 | 0.1102 | 0.9347 | 1.9115 | 1.9115 | 2 | 2 |
| WCM183 | 11 | 193112 | 20104762 | −0.1147 | 0.6295 | 1.9574 | 1.1366 | 2 | 1 |
| WCM183 | 11 | 20112353 | 47266288 | −0.4764 | 0.095 | 1.0681 | 1.0681 | 1 | 1 |
| WCM183 | 11 | 47266439 | 49533487 | −0.1393 | 0.595 | 1.8651 | 1.1559 | 2 | 1 |
| WCM183 | 11 | 49597812 | 82564202 | 0.0823 | 0.8977 | 1.8631 | 1.8631 | 2 | 2 |
| WCM183 | 11 | 82571081 | 111614149 | −0.1112 | 0.6344 | 1.9636 | 1.1409 | 2 | 1 |
| WCM183 | 11 | 111614263 | 120278450 | −0.4796 | 0.09 | 1.0643 | 1.0643 | 1 | 1 |
| WCM183 | 11 | 120280188 | 134605817 | −0.1217 | 0.6197 | 1.9879 | 1.0852 | 2 | 1 |
| WCM183 | 12 | 148427 | 9099034 | 0.0897 | 0.9075 | 1.8758 | 1.8758 | 2 | 2 |
| WCM183 | 12 | 9142297 | 25101862 | −0.5937 | −0.0942 | 1.8247 | 0.0488 | 2 | 0 |
| WCM183 | 12 | 25147248 | 67691593 | 0.0774 | 0.8911 | 1.8546 | 1.8546 | 2 | 2 |
| WCM183 | 12 | 67692761 | 68054306 | 0.3445 | 1.2366 | NA | NA | NA | NA |
| WCM183 | 12 | 68054525 | 85255668 | 0.4953 | 1.4242 | 3.2375 | 2.1299 | 3 | 2 |
| WCM183 | 12 | 85257368 | 133810874 | 0.2776 | 1.1519 | 3.3956 | 1.0483 | 3 | 1 |
| WCM183 | 13 | 19041937 | 28794548 | 0.1255 | 0.9549 | 1.9461 | 1.9308 | 2 | 2 |
| WCM183 | 13 | 28813807 | 80911621 | −0.5158 | 0.0324 | 2.0238 | 0.0217 | 2 | 0 |
| WCM183 | 13 | 80911750 | 115089411 | 0.1373 | 0.9704 | 1.9594 | 1.9594 | 2 | 2 |
| WCM183 | 14 | 19110298 | 61518612 | 0.1033 | 0.9255 | 1.8994 | 1.8994 | 2 | 2 |
| WCM183 | 14 | 61518769 | 97411378 | −0.1297 | 0.6085 | 1.9309 | 1.1186 | 2 | 1 |
| WCM183 | 14 | 97411602 | 107283269 | 0.1757 | 1.0206 | 2.0287 | 2.0287 | 2 | 2 |
| WCM183 | 15 | 20083896 | 37390395 | −0.1233 | 0.6175 | 1.9615 | 1.1069 | 2 | 1 |
| WCM183 | 15 | 37391675 | 45360464 | −0.5048 | 0.05 | 2.0242 | 0.0464 | 2 | 0 |
| WCM183 | 15 | 45361166 | 102506273 | −0.1333 | 0.6035 | 1.9502 | 1.0887 | 2 | 1 |
| WCM183 | 16 | 64396 | 49313348 | 0.1243 | 0.9533 | 1.9362 | 1.9362 | 2 | 2 |
| WCM183 | 16 | 49313526 | 78133986 | −0.5369 | −0.0014 | 1.9759 | 0.0222 | 2 | 0 |
| WCM183 | 16 | 78142332 | 90168697 | 0.1437 | 0.9788 | 1.9708 | 1.9708 | 2 | 2 |
| WCM183 | 17 | 131595 | 21081505 | −0.1691 | 0.553 | 1.8623 | 1.0719 | 2 | 1 |
| WCM183 | 17 | 21087080 | 81187845 | 0.1259 | 0.9554 | 1.9392 | 1.9392 | 2 | 2 |
| WCM183 | 18 | 47607 | 78005197 | −0.1407 | 0.5931 | 1.9115 | 1.1054 | 2 | 1 |
| WCM183 | 19 | 105041 | 8553924 | −0.1615 | 0.5637 | 1.8754 | 1.0808 | 2 | 1 |
| WCM183 | 19 | 8555078 | 37441980 | 0.1079 | 0.9317 | 1.9075 | 1.9075 | 2 | 2 |
| WCM183 | 19 | 37464375 | 59093696 | −0.1516 | 0.5778 | 1.8926 | 1.0925 | 2 | 1 |
| WCM183 | 2 | 41509 | 16747054 | 0.1011 | 0.9226 | 1.8955 | 1.8955 | 2 | 2 |
| WCM183 | 2 | 16769307 | 25377002 | −0.1271 | 0.6122 | 1.7856 | 1.2716 | 2 | 1 |
| WCM183 | 2 | 25377265 | 36774348 | 0.1336 | 0.9654 | 1.9527 | 1.9527 | 2 | 2 |
| WCM183 | 2 | 36775533 | 39988500 | −0.1952 | 0.5158 | 1.8179 | 1.0417 | 2 | 1 |
| WCM183 | 2 | 39993167 | 40670651 | −0.0276 | 0.7498 | NA | NA | NA | NA |
| WCM183 | 2 | 41823623 | 49510173 | −0.5173 | 0.0301 | 2.0047 | 0.0374 | 2 | 0 |
| WCM183 | 2 | 50106501 | 51259342 | 0.2472 | 1.1129 | NA | NA | NA | NA |
| WCM183 | 2 | 51662287 | 64751619 | −0.6046 | −0.1123 | 1.8402 | 0.01 | 2 | 0 |
| WCM183 | 2 | 64778706 | 68480319 | −0.1088 | 0.6378 | 1.8965 | 1.2155 | 2 | 1 |
| WCM183 | 2 | 68483305 | 77747048 | 0.1415 | 0.9758 | 1.9668 | 1.9668 | 2 | 2 |
| WCM183 | 2 | 77748891 | 88885534 | 0.3644 | 1.2617 | 2.5123 | 2.2834 | 3 | 2 |
| WCM183 | 2 | 88885602 | 97031593 | 0.1521 | 0.9898 | 2.0613 | 1.9105 | 2 | 2 |
| WCM183 | 2 | 97032943 | 116548640 | −0.1467 | 0.5847 | 1.8335 | 1.1659 | 2 | 1 |
| WCM183 | 2 | 116572572 | 133066879 | −0.5116 | 0.0392 | 1.9937 | 0.0613 | 2 | 0 |
| WCM183 | 2 | 133070556 | 133175426 | 0.0316 | 0.83 | 1.8637 | 1.6916 | 2 | 2 |
| WCM183 | 2 | 133281690 | 134884753 | −1.9884 | NA | NA | NA | NA | NA |
| WCM183 | 2 | 135011869 | 138434696 | −0.5099 | 0.042 | 2.0084 | 0.0506 | 2 | 0 |
| WCM183 | 2 | 138435203 | 138772143 | −1.8363 | −4.572 | NA | NA | NA | NA |
| WCM183 | 2 | 139307742 | 142012147 | −0.4207 | 0.1818 | 2.2594 | 0.0091 | 2 | 0 |
| WCM183 | 2 | 142238113 | 239033901 | −0.1137 | 0.6309 | 1.8857 | 1.2114 | 2 | 1 |
| WCM183 | 2 | 239034076 | 243037091 | 0.2306 | 1.0917 | 2.9791 | 1.2833 | 3 | 1 |
| WCM183 | 20 | 68337 | 62959285 | 0.169 | 1.0119 | 2.0165 | 2.0165 | 2 | 2 |
| WCM183 | 21 | 9590305 | 48084214 | 0.1157 | 0.9419 | 1.9211 | 1.9211 | 2 | 2 |
| WCM183 | 22 | 16159046 | 38879562 | 0.1859 | 1.0338 | 2.0474 | 2.0474 | 2 | 2 |
| WCM183 | 22 | 38880092 | 51115065 | −0.1657 | 0.5578 | 1.8681 | 1.0759 | 2 | 1 |
| WCM183 | 22 | 51117137 | 51223730 | 0.0596 | 0.8675 | NA | NA | NA | NA |
| WCM183 | 3 | 239472 | 47165993 | 0.4374 | 1.3528 | 2.5541 | 2.5541 | 3 | 3 |
| WCM183 | 3 | 47168108 | 57743471 | −0.1094 | 0.6369 | 2.0102 | 1.0997 | 2 | 1 |
| WCM183 | 3 | 57817266 | 79582217 | 0.3404 | 1.2316 | 2.5822 | 2.1143 | 3 | 2 |
| WCM183 | 3 | 79610945 | 99514629 | 0.0611 | 0.8694 | 1.8782 | 1.7757 | 2 | 2 |
| WCM183 | 3 | 99514776 | 119886023 | −0.1334 | 0.6033 | 1.9243 | 1.1141 | 2 | 1 |
| WCM183 | 3 | 119886306 | 124114050 | 0.1787 | 1.0244 | 2.0342 | 2.0342 | 2 | 2 |
| WCM183 | 3 | 124114560 | 137484312 | 0.4803 | 1.4058 | 2.7051 | 2.5942 | 3 | 3 |
| WCM183 | 3 | 137717678 | 195508387 | 0.7206 | 1.6964 | 4.2197 | 2.262 | 4 | 2 |
| WCM183 | 3 | 195509052 | 197896705 | 0.5388 | 1.4776 | 3.5765 | 1.9931 | 4 | 2 |
| WCM183 | 4 | 53344 | 52938243 | −0.1517 | 0.5776 | 1.8924 | 1.0924 | 2 | 1 |
| WCM183 | 4 | 52942976 | 61529800 | 0.1646 | 1.0061 | 2.0084 | 2.0084 | 2 | 2 |
| WCM183 | 4 | 62066709 | 105412852 | −0.1284 | 0.6104 | 1.8604 | 1.1929 | 2 | 1 |
| WCM183 | 4 | 106155087 | 178897062 | 0.186 | 1.034 | 2.0477 | 2.0477 | 2 | 2 |
| WCM183 | 4 | 178911676 | 190878582 | −0.1772 | 0.5415 | 1.834 | 1.077 | 2 | 1 |
| WCM183 | 5 | 90684 | 10761154 | −0.1259 | 0.6138 | 1.869 | 1.1917 | 2 | 1 |
| WCM183 | 5 | 10972012 | 45645593 | 0.1273 | 0.9572 | 1.9415 | 1.9415 | 2 | 2 |
| WCM183 | 5 | 45695914 | 52405567 | −0.5636 | −0.0447 | NA | NA | NA | NA |
| WCM183 | 5 | 52778714 | 58285608 | 0.1273 | 0.9572 | 1.9415 | 1.9415 | 2 | 2 |
| WCM183 | 5 | 58285786 | 83362298 | −0.1741 | 0.5458 | 1.8537 | 1.066 | 2 | 1 |
| WCM183 | 5 | 83402668 | 101774480 | −0.5981 | −0.1015 | 1.8652 | −0.0011 | 2 | 0 |
| WCM183 | 5 | 101794080 | 109065224 | −1.9304 | −10.5279 | 0.0534 | −0.0521 | 0 | 0 |
| WCM183 | 5 | 109091146 | 112128141 | −0.5779 | −0.0681 | 1.9473 | −0.0394 | 2 | 0 |
| WCM183 | 5 | 112137014 | 112164713 | −1.8235 | −4.3854 | NA | NA | NA | NA |
| WCM183 | 5 | 112170629 | 112222759 | −0.4629 | 0.1162 | NA | NA | NA | NA |
| WCM183 | 5 | 112222891 | 112257908 | −1.7604 | −3.6913 | NA | NA | NA | NA |
| WCM183 | 5 | 112312528 | 121726778 | −0.5668 | −0.0499 | 1.9544 | −0.0224 | 2 | 0 |
| WCM183 | 5 | 121736703 | 180687808 | 0.1499 | 0.9869 | 1.9819 | 1.9819 | 2 | 2 |
| WCM183 | 6 | 203466 | 7559668 | 0.104 | 0.9265 | 2.813 | 0.9882 | 3 | 1 |
| WCM183 | 6 | 7562866 | 41765419 | −0.1823 | 0.5343 | 1.8398 | 1.0566 | 2 | 1 |
| WCM183 | 6 | 41765587 | 66115283 | 0.1557 | 0.9945 | 1.9924 | 1.9924 | 2 | 2 |
| WCM183 | 6 | 66200400 | 97051569 | −0.5334 | 0.0043 | 1.9693 | 0.0367 | 2 | 0 |
| WCM183 | 6 | 97051660 | 120336327 | −0.1732 | 0.5472 | 2.8571 | 0.0653 | 3 | 0 |
| WCM183 | 6 | 121401995 | 121770714 | 0.3162 | 1.2009 | 2.6308 | 1.9669 | 3 | 2 |
| WCM183 | 6 | 122720841 | 127476429 | −0.1416 | 0.5919 | 2.8811 | 0.1334 | 3 | 0 |
| WCM183 | 6 | 127476634 | 154431565 | 0.387 | 1.29 | 2.5615 | 2.3291 | 3 | 2 |
| WCM183 | 6 | 154439865 | 165690063 | 0.096 | 0.9159 | 1.8867 | 1.8867 | 2 | 2 |
| WCM183 | 6 | 165693624 | 170892848 | 0.3262 | 1.2137 | 2.3662 | 2.2723 | 2 | 2 |
| WCM183 | 7 | 193253 | 8792598 | −0.4748 | 0.0976 | 1.0868 | 1.0532 | 1 | 1 |
| WCM183 | 7 | 9654607 | 11022012 | 0.4141 | 1.3239 | NA | NA | NA | NA |
| WCM183 | 7 | 11022392 | 24849598 | 0.3903 | 1.2941 | 3.6355 | 1.2691 | 4 | 1 |
| WCM183 | 7 | 24854765 | 43283513 | 0.2198 | 1.0777 | 2.936 | 1.2855 | 3 | 1 |
| WCM183 | 7 | 43351320 | 53861130 | 0.4459 | 1.3633 | 2.6747 | 2.4708 | 3 | 2 |
| WCM183 | 7 | 53879272 | 75988025 | 0.1764 | 1.0214 | 2.0669 | 1.9929 | 2 | 2 |
| WCM183 | 7 | 76018802 | 94937446 | 0.36 | 1.2562 | 2.8148 | 1.9624 | 3 | 2 |
| WCM183 | 7 | 94940801 | 95167113 | 0.246 | 1.1114 | NA | NA | NA | NA |
| WCM183 | 7 | 95213059 | 103629730 | 0.1373 | 0.9704 | 2.0134 | 1.9053 | 2 | 2 |
| WCM183 | 7 | 103766940 | 128446479 | −0.1588 | 0.5675 | 1.8339 | 1.1301 | 2 | 1 |
| WCM183 | 7 | 128446905 | 158937203 | 0.1513 | 0.9886 | 1.9843 | 1.9843 | 2 | 2 |
| WCM183 | 8 | 163432 | 27204931 | −0.5186 | 0.0281 | 1.9959 | 0.0434 | 2 | 0 |
| WCM183 | 8 | 27255232 | 56962567 | 0.1205 | 0.9483 | 1.9296 | 1.9296 | 2 | 2 |
| WCM183 | 8 | 56982316 | 69243580 | −0.1341 | 0.6024 | 1.5971 | 1.4394 | 2 | 1 |
| WCM183 | 8 | 69351789 | 69730485 | 0.0422 | 0.8442 | NA | NA | NA | NA |
| WCM183 | 8 | 69730697 | 75617961 | 0.3557 | 1.2508 | 3.6217 | 1.1378 | 4 | 1 |
| WCM183 | 8 | 75737595 | 76402304 | −1.2571 | −1.4518 | NA | NA | NA | NA |
| WCM183 | 8 | 76452313 | 76698796 | 0.4089 | 1.3174 | NA | NA | NA | NA |
| WCM183 | 8 | 77593626 | 79717166 | −1.0698 | −0.9965 | NA | NA | NA | NA |
| WCM183 | 8 | 80549046 | 81555368 | 0.1582 | 0.9977 | NA | NA | NA | NA |
| WCM183 | 8 | 81557185 | 90796265 | 0.4077 | 1.3159 | 3.7523 | 1.2269 | 4 | 1 |
| WCM183 | 8 | 90798879 | 113267681 | 0.7247 | 1.7013 | 5.308 | 1.1958 | 5 | 1 |
| WCM183 | 8 | 113275894 | 127564852 | 0.9297 | 1.9418 | 6.4837 | 1.2002 | 6 | 1 |
| WCM183 | 8 | 127565184 | 146203769 | 1.0803 | 2.1151 | 6.5376 | 2.1267 | 7 | 2 |
| WCM183 | 8 | 146220279 | 146279368 | 0.1008 | 0.9222 | NA | NA | NA | NA |
| WCM183 | 9 | 14867 | 9766911 | −0.1585 | 0.568 | 1.8366 | 1.1283 | 2 | 1 |
| WCM183 | 9 | 12694079 | 33342786 | −0.4954 | 0.0651 | 1.0461 | 1.0461 | 1 | 1 |
| WCM183 | 9 | 33344104 | 78547260 | −0.141 | 0.5926 | 1.9152 | 1.1008 | 2 | 1 |
| WCM183 | 9 | 78601032 | 141107539 | 0.1415 | 0.9758 | 2.8438 | 1.0897 | 3 | 1 |
| WCM183 | X | 2700156 | 10535265 | 0.2168 | 1.0738 | NA | NA | NA | NA |
| WCM183 | X | 10588654 | 31241136 | −0.4759 | 0.0958 | NA | NA | NA | NA |
| WCM183 | X | 31279075 | 154507335 | 0.1994 | 1.0513 | NA | NA | NA | NA |
| WCM183_Z4_1 | 1 | 69603 | 16774618 | −0.8205 | 0.0697 | 2.0414 | 0.0576 | 2 | 0 |
| WCM183_Z4_1 | 1 | 16775691 | 17296801 | −0.6229 | 0.2746 | 1.5225 | 0.8968 | 2 | 1 |
| WCM183_Z4_1 | 1 | 17297200 | 22852746 | −0.7991 | 0.092 | 2.087 | 0.0447 | 2 | 0 |
| WCM183_Z4_1 | 1 | 22895820 | 64698324 | −0.3046 | 0.6027 | 2.0272 | 1.0099 | 2 | 1 |
| WCM183_Z4_1 | 1 | 64707356 | 75055585 | −0.8391 | 0.0504 | 1.1724 | 0.8988 | 1 | 1 |
| WCM183_Z4_1 | 1 | 75065394 | 85547006 | −0.3348 | 0.5717 | 1.8973 | 1.0753 | 2 | 1 |
| WCM183_Z4_1 | 1 | 85548079 | 91851307 | −0.8645 | 0.024 | 1.1501 | 0.8834 | 1 | 1 |
| WCM183_Z4_1 | 1 | 91859761 | 115829271 | −0.3181 | 0.5889 | 1.957 | 1.0512 | 2 | 1 |
| WCM183_Z4_1 | 1 | 116194051 | 151506526 | 0.0877 | 1.0045 | 2.2089 | 1.8035 | 2 | 2 |
| WCM183_Z4_1 | 1 | 151507566 | 155452212 | 0.3931 | 1.3155 | 3.0685 | 1.9094 | 3 | 2 |
| WCM183_Z4_1 | 1 | 155490984 | 158450552 | −0.8303 | 0.0595 | 2.0349 | 0.0494 | 2 | 0 |
| WCM183_Z4_1 | 1 | 158507184 | 212002477 | 0.0902 | 1.007 | 2.0241 | 1.9954 | 2 | 2 |
| WCM183_Z4_1 | 1 | 212115362 | 215741053 | −0.8156 | 0.0749 | 2.065 | 0.0415 | 2 | 0 |
| WCM183_Z4_1 | 1 | 215747180 | 249212354 | 0.0855 | 1.0022 | 2.0132 | 1.9928 | 2 | 2 |
| WCM183_Z4_1 | 10 | 226016 | 30748399 | −0.8534 | 0.0355 | 1.9913 | 0.0586 | 2 | 0 |
| WCM183_Z4_1 | 10 | 30749763 | 39147119 | 0.0358 | 0.9515 | 2.0821 | 1.7857 | 2 | 2 |
| WCM183_Z4_1 | 10 | 42905578 | 53059393 | −0.8348 | 0.0549 | 2.0069 | 0.0707 | 2 | 0 |
| WCM183_Z4_1 | 10 | 53227568 | 134699362 | 0.0526 | 0.9686 | 1.9569 | 1.9569 | 2 | 2 |
| WCM183_Z4_1 | 10 | 134705908 | 135087709 | −0.3005 | 0.6069 | 2.0268 | 1.0192 | 2 | 1 |
| WCM183_Z4_1 | 10 | 135088309 | 135440105 | −0.3444 | 0.5619 | 1.9907 | 0.9616 | 2 | 1 |
| WCM183_Z4_1 | 11 | 193112 | 47203976 | −0.8033 | 0.0876 | 1.1907 | 0.9345 | 1 | 1 |
| WCM183_Z4_1 | 11 | 47204270 | 48346791 | −0.2719 | 0.6364 | 1.9151 | 1.1937 | 2 | 1 |
| WCM183_Z4_1 | 11 | 48347067 | 56431977 | −0.0353 | 0.8788 | 2.0457 | 1.632 | 2 | 2 |
| WCM183_Z4_1 | 11 | 56467926 | 78516507 | 0.3868 | 1.3092 | 2.9517 | 2.0043 | 3 | 2 |
| WCM183_Z4_1 | 11 | 78523167 | 110007479 | 0.0811 | 0.9978 | 2.9815 | 1.0123 | 3 | 1 |
| WCM183_Z4_1 | 11 | 110007857 | 111635566 | −0.3339 | 0.5727 | NA | NA | NA | NA |
| WCM183_Z4_1 | 11 | 111706902 | 131530906 | −0.8157 | 0.0748 | 1.0762 | 1.0302 | 1 | 1 |
| WCM183_Z4_1 | 11 | 132016197 | 134856498 | −0.2787 | 0.6294 | NA | NA | NA | NA |
| WCM183_Z4_1 | 12 | 148811 | 9147765 | 0.0869 | 1.0036 | 2.0357 | 1.9743 | 2 | 2 |
| WCM183_Z4_1 | 12 | 9161648 | 25101865 | −0.827 | 0.063 | 2.0516 | 0.0376 | 2 | 0 |
| WCM183_Z4_1 | 12 | 25147250 | 67688883 | 0.0811 | 0.9977 | 2.0152 | 1.9785 | 2 | 2 |
| WCM183_Z4_1 | 12 | 67691075 | 72164395 | 0.3545 | 1.2764 | 2.92 | 1.9245 | 3 | 2 |
| WCM183_Z4_1 | 12 | 72167794 | 85255675 | 0.3266 | 1.2479 | 2.9526 | 1.7974 | 3 | 2 |
| WCM183_Z4_1 | 12 | 85260923 | 133733253 | 0.078 | 0.9946 | 2.9445 | 1.0405 | 3 | 1 |
| WCM183_Z4_1 | 12 | 133733526 | 133810735 | 0.1971 | 1.1161 | NA | NA | NA | NA |
| WCM183_Z4_1 | 13 | 19600515 | 28794416 | 0.0581 | 0.9742 | 2.0208 | 1.9084 | 2 | 2 |
| WCM183_Z4_1 | 13 | 28813807 | 82264468 | −0.8262 | 0.0638 | 2.0169 | 0.0735 | 2 | 0 |
| WCM183_Z4_1 | 13 | 82264533 | 115089411 | 0.0931 | 1.01 | 2.0386 | 1.9892 | 2 | 2 |
| WCM183_Z4_1 | 14 | 19377716 | 63174760 | 0.0748 | 0.9913 | 2.0083 | 1.9677 | 2 | 2 |
| WCM183_Z4_1 | 14 | 63175007 | 98444438 | −0.2921 | 0.6156 | 2.0107 | 1.0538 | 2 | 1 |
| WCM183_Z4_1 | 14 | 99182569 | 107283027 | 0.1142 | 1.0315 | 2.0838 | 2.0045 | 2 | 2 |
| WCM183_Z4_1 | 15 | 20170028 | 26260619 | −0.2532 | 0.6555 | 2.1282 | 1.0222 | 2 | 1 |
| WCM183_Z4_1 | 15 | 26793033 | 45951336 | −0.8224 | 0.0678 | 2.0257 | 0.0706 | 2 | 0 |
| WCM183_Z4_1 | 15 | 45954219 | 102506282 | −0.2911 | 0.6167 | 3.0138 | 0.0528 | 3 | 0 |
| WCM183_Z4_1 | 16 | 66953 | 48643797 | 0.0995 | 1.0166 | 2.0437 | 2.0024 | 2 | 2 |
| WCM183_Z4_1 | 16 | 49193569 | 78085854 | −0.8212 | 0.0691 | 2.0503 | 0.0478 | 2 | 0 |
| WCM183_Z4_1 | 16 | 78133757 | 90162178 | 0.0887 | 1.0055 | 2.0077 | 2.0077 | 2 | 2 |
| WCM183_Z4_1 | 17 | 131595 | 21215537 | −0.2955 | 0.6121 | 1.965 | 1.092 | 2 | 1 |
| WCM183_Z4_1 | 17 | 21216846 | 45050443 | 0.0949 | 1.0118 | 2.0618 | 1.9712 | 2 | 2 |
| WCM183_Z4_1 | 17 | 45056177 | 81083455 | 0.4004 | 1.323 | 2.9774 | 2.0262 | 3 | 2 |
| WCM183_Z4_1 | 18 | 48247 | 78005201 | −0.3053 | 0.602 | 2.02 | 1.0157 | 2 | 1 |
| WCM183_Z4_1 | 19 | 281498 | 37660744 | 0.0935 | 1.0103 | 2.035 | 1.9938 | 2 | 2 |
| WCM183_Z4_1 | 19 | 37676251 | 59083951 | −0.2883 | 0.6195 | 2.0319 | 1.0409 | 2 | 1 |
| WCM183_Z4_1 | 2 | 41609 | 11721146 | −0.291 | 0.6168 | 2.0894 | 0.9774 | 2 | 1 |
| WCM183_Z4_1 | 2 | 11725320 | 40657378 | 0.0819 | 0.9985 | 2.0265 | 1.9694 | 2 | 2 |
| WCM183_Z4_1 | 2 | 42165742 | 49510173 | −0.8376 | 0.052 | 1.9931 | 0.0802 | 2 | 0 |
| WCM183_Z4_1 | 2 | 50149331 | 53832013 | 0.0513 | 0.9673 | NA | NA | NA | NA |
| WCM183_Z4_1 | 2 | 53897735 | 64685492 | −0.8461 | 0.0431 | 2.0466 | 0.0141 | 2 | 0 |
| WCM183_Z4_1 | 2 | 64778623 | 65593739 | 0.1324 | 1.0501 | 2.1361 | 2.0051 | 2 | 2 |
| WCM183_Z4_1 | 2 | 65604767 | 88895343 | 0.3981 | 1.3207 | 3.0133 | 1.9823 | 3 | 2 |
| WCM183_Z4_1 | 2 | 88913369 | 116548918 | 0.0823 | 0.9989 | 2.0454 | 1.9516 | 2 | 2 |
| WCM183_Z4_1 | 2 | 116572408 | 133175429 | −0.831 | 0.0588 | 2.023 | 0.0602 | 2 | 0 |
| WCM183_Z4_1 | 2 | 133403772 | 134884754 | −3.7835 | −3.3469 | NA | NA | NA | NA |
| WCM183_Z4_1 | 2 | 135012032 | 142012149 | −0.8281 | 0.0619 | 2.037 | 0.0506 | 2 | 0 |
| WCM183_Z4_1 | 2 | 142237996 | 239079602 | 0.0704 | 0.9868 | 2.002 | 1.9615 | 2 | 2 |
| WCM183_Z4_1 | 2 | 239090708 | 242946607 | 0.404 | 1.3267 | 3.0131 | 2.0034 | 3 | 2 |
| WCM183_Z4_1 | 20 | 68363 | 10035223 | 0.0905 | 1.0073 | 2.0614 | 1.9588 | 2 | 2 |
| WCM183_Z4_1 | 20 | 10036202 | 14307161 | −0.2999 | 0.6076 | NA | NA | NA | NA |
| WCM183_Z4_1 | 20 | 14307388 | 37601243 | 0.1198 | 1.0373 | 2.0858 | 2.0188 | 2 | 2 |
| WCM183_Z4_1 | 20 | 37612399 | 37667181 | −0.872 | 0.0162 | NA | NA | NA | NA |
| WCM183_Z4_1 | 20 | 39316581 | 62904848 | 0.112 | 1.0292 | 2.041 | 2.041 | 2 | 2 |
| WCM183_Z4_1 | 21 | 9826205 | 48084211 | 0.0817 | 0.9983 | 2.018 | 1.9773 | 2 | 2 |
| WCM183_Z4_1 | 22 | 16287443 | 39267726 | 0.0988 | 1.0157 | 2.0426 | 2.0013 | 2 | 2 |
| WCM183_Z4_1 | 22 | 39353704 | 51215138 | −0.2853 | 0.6226 | 2.0521 | 1.0273 | 2 | 1 |
| WCM183_Z4_1 | 3 | 239347 | 3084004 | 0.3389 | 1.2605 | NA | NA | NA | NA |
| WCM183_Z4_1 | 3 | 3084747 | 9066960 | 0.6307 | 1.5568 | 3.0017 | 2.8823 | 3 | 3 |
| WCM183_Z4_1 | 3 | 9068649 | 47165118 | 0.388 | 1.3104 | 2.9845 | 1.9758 | 3 | 2 |
| WCM183_Z4_1 | 3 | 47165651 | 57678597 | −0.286 | 0.6219 | 2.0478 | 1.03 | 2 | 1 |
| WCM183_Z4_1 | 3 | 57743550 | 79639015 | 0.3658 | 1.2879 | 2.916 | 1.9673 | 3 | 2 |
| WCM183_Z4_1 | 3 | 81539611 | 81792714 | −0.947 | −0.062 | NA | NA | NA | NA |
| WCM183_Z4_1 | 3 | 81810770 | 98600581 | −0.056 | 0.8576 | 2.027 | 1.597 | 2 | 2 |
| WCM183_Z4_1 | 3 | 98620161 | 119962621 | −0.3258 | 0.581 | 1.9956 | 0.9962 | 2 | 1 |
| WCM183_Z4_1 | 3 | 119962869 | 124193571 | 0.0885 | 1.0053 | 2.0914 | 1.9234 | 2 | 2 |
| WCM183_Z4_1 | 3 | 124196174 | 137484072 | 0.394 | 1.3165 | 2.9362 | 2.0449 | 3 | 2 |
| WCM183_Z4_1 | 3 | 137484257 | 197894713 | 0.625 | 1.5511 | 3.9557 | 1.905 | 4 | 2 |
| WCM183_Z4_1 | 4 | 59339 | 52960717 | −0.3141 | 0.593 | 1.9888 | 1.0278 | 2 | 1 |
| WCM183_Z4_1 | 4 | 53463887 | 57907033 | 0.3575 | 1.2794 | 3.0249 | 1.8299 | 3 | 2 |
| WCM183_Z4_1 | 4 | 57976292 | 102269630 | 0.0702 | 0.9866 | 2.0199 | 1.943 | 2 | 2 |
| WCM183_Z4_1 | 4 | 102269801 | 103978998 | 0.2954 | 1.2162 | NA | NA | NA | NA |
| WCM183_Z4_1 | 4 | 103987659 | 104027377 | −0.9997 | −0.117 | NA | NA | NA | NA |
| WCM183_Z4_1 | 4 | 104030207 | 104079831 | 0.2955 | 1.2162 | NA | NA | NA | NA |
| WCM183_Z4_1 | 4 | 104080281 | 108615102 | 0.5945 | 1.5201 | NA | NA | NA | NA |
| WCM183_Z4_1 | 4 | 108622411 | 118005832 | 0.3408 | 1.2624 | NA | NA | NA | NA |
| WCM183_Z4_1 | 4 | 118006183 | 126242679 | 0.6 | 1.5257 | 2.9873 | 2.7711 | 3 | 3 |
| WCM183_Z4_1 | 4 | 126315100 | 126337716 | −0.2466 | 0.6623 | NA | NA | NA | NA |
| WCM183_Z4_1 | 4 | 126355420 | 178911657 | 0.6395 | 1.5658 | 3.0264 | 2.8943 | 3 | 3 |
| WCM183_Z4_1 | 4 | 183090510 | 190426471 | 0.08 | 0.9966 | 3.0098 | 0.9809 | 3 | 1 |
| WCM183_Z4_1 | 5 | 140532 | 11903945 | −0.3016 | 0.6058 | 2.0128 | 1.0309 | 2 | 1 |
| WCM183_Z4_1 | 5 | 13692093 | 45645328 | 0.0796 | 0.9962 | 2.0274 | 1.9622 | 2 | 2 |
| WCM183_Z4_1 | 5 | 45695964 | 52900714 | −0.7653 | 0.1271 | NA | NA | NA | NA |
| WCM183_Z4_1 | 5 | 52942197 | 58021861 | 0.0748 | 0.9913 | 2.1422 | 1.8337 | 2 | 2 |
| WCM183_Z4_1 | 5 | 58120931 | 83549998 | −0.3092 | 0.598 | 1.9522 | 1.075 | 2 | 1 |
| WCM183_Z4_1 | 5 | 83680169 | 101774388 | −0.9033 | −0.0164 | 1.9613 | 0.016 | 2 | 0 |
| WCM183_Z4_1 | 5 | 101811461 | 109049411 | −3.2844 | −2.6793 | NA | NA | NA | NA |
| WCM183_Z4_1 | 5 | 109091107 | 121518257 | −0.8618 | 0.0268 | 2.0197 | 0.0177 | 2 | 0 |
| WCM183_Z4_1 | 5 | 121726864 | 180687769 | 0.0785 | 0.9951 | 2.0115 | 1.9749 | 2 | 2 |
| WCM183_Z4_1 | 6 | 203588 | 8428236 | 0.0591 | 0.9753 | 3.0241 | 0.908 | 3 | 1 |
| WCM183_Z4_1 | 6 | 8430049 | 41721696 | −0.3021 | 0.6054 | 2.0293 | 1.0134 | 2 | 1 |
| WCM183_Z4_1 | 6 | 41738429 | 50810865 | 0.0972 | 1.0142 | 2.0672 | 1.9723 | 2 | 2 |
| WCM183_Z4_1 | 6 | 51483949 | 55113575 | 0.3372 | 1.2587 | 2.4463 | 2.3392 | 2 | 2 |
| WCM183_Z4_1 | 6 | 55120051 | 66115210 | 0.0984 | 1.0154 | 2.0958 | 1.9472 | 2 | 2 |
| WCM183_Z4_1 | 6 | 66200507 | 97001296 | −0.8624 | 0.0261 | 1.9902 | 0.0463 | 2 | 0 |
| WCM183_Z4_1 | 6 | 97051527 | 127471675 | −0.316 | 0.591 | 2.9753 | 0.0373 | 3 | 0 |
| WCM183_Z4_1 | 6 | 127476490 | 143655236 | 0.3845 | 1.3068 | 2.8236 | 2.1243 | 3 | 2 |
| WCM183_Z4_1 | 6 | 143656207 | 153323820 | 0.0764 | 0.9929 | 3.0447 | 0.9357 | 3 | 1 |
| WCM183_Z4_1 | 6 | 153332748 | 165715270 | −0.287 | 0.6208 | 2.108 | 0.9676 | 2 | 1 |
| WCM183_Z4_1 | 6 | 165715556 | 170893427 | 0.3106 | 1.2317 | 2.7045 | 1.9923 | 3 | 2 |
| WCM183_Z4_1 | 7 | 193451 | 8196529 | −0.2798 | 0.6283 | 2.0997 | 0.9917 | 2 | 1 |
| WCM183_Z4_1 | 7 | 8196810 | 51261169 | 0.3825 | 1.3048 | 2.9252 | 2.0158 | 3 | 2 |
| WCM183_Z4_1 | 7 | 51287453 | 69064936 | 0.0833 | 1 | 2.0775 | 1.9224 | 2 | 2 |
| WCM183_Z4_1 | 7 | 69364361 | 94919406 | 0.3649 | 1.2869 | 2.9017 | 1.9785 | 3 | 2 |
| WCM183_Z4_1 | 7 | 94928327 | 103389942 | 0.0988 | 1.0158 | 2.0963 | 1.9478 | 2 | 2 |
| WCM183_Z4_1 | 7 | 103393636 | 158937400 | −0.2952 | 0.6124 | 2.0063 | 1.0514 | 2 | 1 |
| WCM183_Z4_1 | 8 | 190906 | 27204941 | −0.4027 | 0.5018 | 2.7717 | 0.0604 | 3 | 0 |
| WCM183_Z4_1 | 8 | 27255276 | 69730480 | 0.3423 | 1.2639 | 2.7924 | 2.0106 | 3 | 2 |
| WCM183_Z4_1 | 8 | 70476335 | 75272466 | 0.8077 | 1.7362 | NA | NA | NA | NA |
| WCM183_Z4_1 | 8 | 75274141 | 76476254 | 0.2562 | 1.1763 | NA | NA | NA | NA |
| WCM183_Z4_1 | 8 | 77616564 | 79717152 | −0.3701 | 0.5355 | NA | NA | NA | NA |
| WCM183_Z4_1 | 8 | 80523445 | 89340141 | 0.806 | 1.7345 | 3.4523 | 3.203 | 3 | 3 |
| WCM183_Z4_1 | 8 | 90770353 | 113237068 | 1.1568 | 2.0892 | 5.7503 | 2.7598 | 6 | 3 |
| WCM183_Z4_1 | 8 | 113241088 | 146279475 | 1.336 | 2.27 | 6.7545 | 2.8918 | 7 | 3 |
| WCM183_Z4_1 | 9 | 17101 | 8733788 | −0.31 | 0.5972 | 1.9651 | 1.0604 | 2 | 1 |
| WCM183_Z4_1 | 9 | 12694075 | 32987821 | −0.8586 | 0.0301 | 1.1592 | 0.8829 | 1 | 1 |
| WCM183_Z4_1 | 9 | 32988129 | 78686806 | −0.2996 | 0.6079 | 1.9546 | 1.0935 | 2 | 1 |
| WCM183_Z4_1 | 9 | 78710991 | 141111414 | 0.0859 | 1.0026 | 2.9731 | 1.0341 | 3 | 1 |
| WCM183_Z4_1 | X | 2707682 | 10535466 | 0.0641 | 0.9804 | NA | NA | NA | NA |
| WCM183_Z4_1 | X | 11130185 | 31279075 | −0.8632 | 0.0253 | NA | NA | NA | NA |
| WCM183_Z4_1 | X | 31284937 | 154509348 | 0.0437 | 0.9595 | NA | NA | NA | NA |
| WCM185 | 1 | 17484 | 249213973 | −0.0128 | NA | NA | NA | NA | NA |
| WCM185 | 10 | 225936 | 135438983 | −0.0244 | NA | NA | NA | NA | NA |
| WCM185 | 11 | 193112 | 134605916 | −0.0303 | NA | NA | NA | NA | NA |
| WCM185 | 12 | 148444 | 133810868 | −0.0384 | NA | NA | NA | NA | NA |
| WCM185 | 13 | 19041992 | 115089411 | −0.0449 | NA | NA | NA | NA | NA |
| WCM185 | 14 | 19110239 | 107283184 | −0.0427 | NA | NA | NA | NA | NA |
| WCM185 | 15 | 20083897 | 102514189 | −0.0092 | NA | NA | NA | NA | NA |
| WCM185 | 16 | 67688 | 90168687 | −0.058 | NA | NA | NA | NA | NA |
| WCM185 | 17 | 131595 | 81052563 | −0.0285 | NA | NA | NA | NA | NA |
| WCM185 | 18 | 47732 | 78005164 | −0.0253 | NA | NA | NA | NA | NA |
| WCM185 | 19 | 105080 | 59093645 | −0.0105 | NA | NA | NA | NA | NA |
| WCM185 | 2 | 41463 | 243036958 | −0.0228 | NA | NA | NA | NA | NA |
| WCM185 | 20 | 68342 | 62934617 | −1.00E−04 | NA | NA | NA | NA | NA |
| WCM185 | 21 | 9590311 | 48084201 | −0.0337 | NA | NA | NA | NA | NA |
| WCM185 | 22 | 16159016 | 51193629 | −0.0032 | NA | NA | NA | NA | NA |
| WCM185 | 3 | 239472 | 197896724 | −0.0339 | NA | NA | NA | NA | NA |
| WCM185 | 4 | 53360 | 190878508 | −0.0593 | NA | NA | NA | NA | NA |
| WCM185 | 5 | 90714 | 180687827 | −0.0308 | NA | NA | NA | NA | NA |
| WCM185 | 6 | 203424 | 170892848 | −0.057 | NA | NA | NA | NA | NA |
| WCM185 | 7 | 193253 | 158937161 | −0.0024 | NA | NA | NA | NA | NA |
| WCM185 | 8 | 190879 | 146279480 | −0.0485 | NA | NA | NA | NA | NA |
| WCM185 | 9 | 116874 | 141071646 | 0.0096 | NA | NA | NA | NA | NA |
| WCM185 | X | 2700139 | 154490227 | −0.0771 | NA | NA | NA | NA | NA |
| WCM185-TP1 | 1 | 17459 | 215808056 | −0.1144 | 0.0209 | 1.0146 | 1.0146 | 1 | 1 |
| WCM185-TP1 | 1 | 215812492 | 243595136 | −0.3443 | −2.7772 | 0.1459 | 0.1459 | 0 | 0 |
| WCM185-TP1 | 1 | 243651876 | 249213961 | −0.0242 | 0.4803 | 1.395 | 1.395 | 1 | 1 |
| WCM185-TP1 | 10 | 226106 | 135439032 | −0.0957 | 0.1264 | 1.0916 | 1.0916 | 1 | 1 |
| WCM185-TP1 | 11 | 193112 | 134605916 | −0.1216 | −0.0216 | 0.9852 | 0.9852 | 1 | 1 |
| WCM185-TP1 | 12 | 148388 | 133810788 | −0.1158 | 0.0128 | 1.0089 | 1.0089 | 1 | 1 |
| WCM185-TP1 | 13 | 19041992 | 115089411 | −0.1178 | 0.0011 | 1.0008 | 1.0008 | 1 | 1 |
| WCM185-TP1 | 14 | 19110394 | 107283210 | −0.1104 | 0.0439 | 1.0309 | 1.0309 | 1 | 1 |
| WCM185-TP1 | 15 | 20083887 | 25277789 | 8.00E−04 | 0.5897 | 1.7732 | 1.2367 | 2 | 1 |
| WCM185-TP1 | 15 | 25287053 | 25599532 | 1.4144 | 3.6009 | NA | NA | NA | NA |
| WCM185-TP1 | 15 | 25599815 | 102514230 | −0.0853 | 0.1827 | 1.135 | 1.135 | 1 | 1 |
| WCM185-TP1 | 16 | 67686 | 72156908 | −0.1261 | −0.0481 | 0.9672 | 0.9672 | 1 | 1 |
| WCM185-TP1 | 16 | 72157544 | 88932017 | −0.3456 | −2.8199 | 0.3376 | −0.0543 | 0 | 0 |
| WCM185-TP1 | 16 | 88941214 | 90168697 | −0.2258 | −0.7949 | 1.1659 | −0.0132 | 1 | 0 |
| WCM185-TP1 | 17 | 131595 | 81052563 | −0.1158 | 0.0128 | 1.0089 | 1.0089 | 1 | 1 |
| WCM185-TP1 | 18 | 47672 | 78005140 | −0.1362 | −0.1104 | 0.9263 | 0.9263 | 1 | 1 |
| WCM185-TP1 | 19 | 105050 | 8982261 | −0.138 | −0.1221 | 0.9189 | 0.9189 | 1 | 1 |
| WCM185-TP1 | 19 | 8986999 | 9768797 | 0.3797 | 1.7755 | 3.4236 | 3.4236 | 3 | 3 |
| WCM185-TP1 | 19 | 9770173 | 11687908 | −0.1806 | −0.4149 | 0.7501 | 0.7501 | 1 | 1 |
| WCM185-TP1 | 19 | 11708361 | 12754426 | 0.7157 | 2.4859 | 5.6018 | 5.6018 | 6 | 6 |
| WCM185-TP1 | 19 | 12754621 | 19773917 | −0.161 | −0.2736 | 0.8273 | 0.8273 | 1 | 1 |
| WCM185-TP1 | 19 | 19779736 | 24310467 | 0.7496 | 2.5486 | 5.8508 | 5.8508 | 6 | 6 |
| WCM185-TP1 | 19 | 24310774 | 43023330 | −0.0724 | 0.2498 | 1.1891 | 1.1891 | 1 | 1 |
| WCM185-TP1 | 19 | 43025383 | 43780550 | 1.3262 | 3.4756 | 11.9406 | 10.308 | 12 | 10 |
| WCM185-TP1 | 19 | 43858026 | 52794481 | −0.0846 | 0.1863 | 1.1378 | 1.1378 | 1 | 1 |
| WCM185-TP1 | 19 | 52795443 | 54265726 | 0.7123 | 2.4795 | 5.577 | 5.577 | 6 | 6 |
| WCM185-TP1 | 19 | 54290959 | 59093696 | 0.0341 | 0.7258 | 1.6539 | 1.6539 | 2 | 2 |
| WCM185-TP1 | 2 | 41623 | 116600728 | −0.0789 | 0.216 | 1.1615 | 1.1615 | 1 | 1 |
| WCM185-TP1 | 2 | 116601631 | 151326761 | −0.3189 | −2.0882 | 0.4092 | 0.0612 | 0 | 0 |
| WCM185-TP1 | 2 | 151328247 | 243036958 | −0.1139 | 0.0237 | 1.0166 | 1.0166 | 1 | 1 |
| WCM185-TP1 | 20 | 68346 | 62934623 | −0.1191 | −0.0067 | 0.9953 | 0.9953 | 1 | 1 |
| WCM185-TP1 | 21 | 9590305 | 48084175 | −0.0913 | 0.1506 | 1.11 | 1.11 | 1 | 1 |
| WCM185-TP1 | 22 | 16159068 | 51193629 | −0.0554 | 0.3346 | 1.2611 | 1.2611 | 1 | 1 |
| WCM185-TP1 | 3 | 239472 | 197896724 | −0.1065 | 0.0666 | 1.0473 | 1.0473 | 1 | 1 |
| WCM185-TP1 | 4 | 53360 | 190878479 | −0.1214 | −0.0199 | 0.9863 | 0.9863 | 1 | 1 |
| WCM185-TP1 | 5 | 90750 | 180687824 | −0.1098 | 0.0477 | 1.0336 | 1.0336 | 1 | 1 |
| WCM185-TP1 | 6 | 203479 | 66005970 | −0.1104 | 0.0443 | 1.0312 | 1.0312 | 1 | 1 |
| WCM185-TP1 | 6 | 66006023 | 126329584 | −0.3688 | −4.0283 | 0.0613 | 0.0613 | 0 | 0 |
| WCM185-TP1 | 6 | 126332349 | 170892848 | −0.1439 | −0.1594 | 0.8954 | 0.8954 | 1 | 1 |
| WCM185-TP1 | 7 | 193222 | 158937135 | 0.0724 | 0.8716 | 1.8297 | 1.8297 | 2 | 2 |
| WCM185-TP1 | 8 | 190878 | 3165420 | −0.1523 | −0.2143 | 0.862 | 0.862 | 1 | 1 |
| WCM185-TP1 | 8 | 3165934 | 35563495 | −0.3393 | −2.6115 | 0.1636 | 0.1636 | 0 | 0 |
| WCM185-TP1 | 8 | 35579010 | 146279498 | −0.0524 | 0.3492 | 1.2738 | 1.2738 | 1 | 1 |
| WCM185-TP1 | 9 | 116918 | 141071646 | 0.0072 | 0.6165 | 1.5331 | 1.5331 | 2 | 2 |
| WCM185-TP1 | X | 2700122 | 154490187 | −0.0835 | 0.1922 | NA | NA | NA | NA |
| WCM185-TP2 | 1 | 17483 | 6605097 | 0.0032 | 0.1046 | 1.0752 | 1.0752 | 1 | 1 |
| WCM185-TP2 | 1 | 6605205 | 60297867 | −0.1647 | −0.1165 | 0.9224 | 0.9224 | 1 | 1 |
| WCM185-TP2 | 1 | 60299038 | 152104268 | −0.0728 | 0.0055 | 1.0038 | 1.0038 | 1 | 1 |
| WCM185-TP2 | 1 | 152127221 | 153122563 | 0.6665 | 0.9162 | 1.8871 | 1.8871 | 2 | 2 |
| WCM185-TP2 | 1 | 153177276 | 185278583 | −0.0867 | −0.0128 | 0.9912 | 0.9912 | 1 | 1 |
| WCM185-TP2 | 1 | 185703968 | 196762536 | −0.7244 | −0.9317 | 0.9602 | 0.0883 | 1 | 0 |
| WCM185-TP2 | 1 | 196763797 | 215808069 | −0.0209 | 0.0734 | 1.0522 | 1.0522 | 1 | 1 |
| WCM185-TP2 | 1 | 215812492 | 243289756 | −0.7235 | −0.9302 | 0.9795 | 0.07 | 1 | 0 |
| WCM185-TP2 | 1 | 243292633 | 249213973 | −0.0315 | 0.0596 | 1.0422 | 1.0422 | 1 | 1 |
| WCM185-TP2 | 10 | 225995 | 135438967 | −0.0716 | 0.0072 | 1.005 | 1.005 | 1 | 1 |
| WCM185-TP2 | 11 | 193112 | 134605916 | −0.1091 | −0.0424 | 0.971 | 0.971 | 1 | 1 |
| WCM185-TP2 | 12 | 148417 | 133810832 | −0.1109 | −0.0447 | 0.9695 | 0.9695 | 1 | 1 |
| WCM185-TP2 | 13 | 19041937 | 115089411 | −0.0862 | −0.0121 | 0.9917 | 0.9917 | 1 | 1 |
| WCM185-TP2 | 14 | 19110394 | 107283228 | −0.0966 | −0.0258 | 0.9823 | 0.9823 | 1 | 1 |
| WCM185-TP2 | 15 | 20083889 | 25287053 | 0.0396 | 0.1515 | 1.1863 | 1.0351 | 1 | 1 |
| WCM185-TP2 | 15 | 25296693 | 25495973 | 1.5249 | 1.879 | NA | NA | NA | NA |
| WCM185-TP2 | 15 | 25514900 | 102514230 | −0.0837 | −0.0089 | 0.9939 | 0.9939 | 1 | 1 |
| WCM185-TP2 | 16 | 67688 | 72184720 | −0.1167 | −0.0525 | 0.9642 | 0.9642 | 1 | 1 |
| WCM185-TP2 | 16 | 72188278 | 90128558 | −0.7445 | −0.964 | 0.9741 | 0.0512 | 1 | 0 |
| WCM185-TP2 | 16 | 90128812 | 90168694 | −0.1228 | −0.0605 | NA | NA | NA | NA |
| WCM185-TP2 | 17 | 131595 | 563578 | −0.1653 | −0.1174 | NA | NA | NA | NA |
| WCM185-TP2 | 17 | 565054 | 14110317 | −0.5413 | −0.6485 | 0.6866 | 0.5893 | 1 | 1 |
| WCM185-TP2 | 17 | 14139572 | 81052563 | −0.1048 | −0.0367 | 0.9749 | 0.9749 | 1 | 1 |
| WCM185-TP2 | 18 | 47808 | 25646481 | −0.0511 | 0.034 | 1.0239 | 1.0239 | 1 | 1 |
| WCM185-TP2 | 18 | 25727739 | 78005197 | −0.4135 | −0.4614 | 0.7648 | 0.6877 | 1 | 1 |
| WCM185-TP2 | 19 | 105021 | 2538224 | −0.1129 | −0.0474 | 0.9741 | 0.9613 | 1 | 1 |
| WCM185-TP2 | 19 | 2538668 | 3985327 | −0.7086 | −0.9064 | 0.9208 | 0.1462 | 1 | 0 |
| WCM185-TP2 | 19 | 4013115 | 8982193 | −0.1197 | −0.0565 | 0.9616 | 0.9616 | 1 | 1 |
| WCM185-TP2 | 19 | 8987037 | 9869186 | 0.4323 | 0.6388 | 1.5571 | 1.5571 | 2 | 2 |
| WCM185-TP2 | 19 | 9869378 | 11687372 | −0.2109 | −0.179 | 0.8833 | 0.8833 | 1 | 1 |
| WCM185-TP2 | 19 | 11687629 | 12754375 | 0.6722 | 0.9228 | 1.8958 | 1.8958 | 2 | 2 |
| WCM185-TP2 | 19 | 12754621 | 19773929 | −0.1514 | −0.0988 | 0.9338 | 0.9338 | 1 | 1 |
| WCM185-TP2 | 19 | 19779759 | 24310198 | 0.8067 | 1.0786 | 2.112 | 2.112 | 2 | 2 |
| WCM185-TP2 | 19 | 24310535 | 43016600 | −0.0992 | −0.0293 | 0.9799 | 0.9799 | 1 | 1 |
| WCM185-TP2 | 19 | 43023114 | 43780474 | 1.2674 | 1.5971 | 3.1791 | 2.8717 | 3 | 3 |
| WCM185-TP2 | 19 | 43858046 | 52772856 | −0.1204 | −0.0573 | 0.961 | 0.961 | 1 | 1 |
| WCM185-TP2 | 19 | 52780808 | 54299079 | 0.7019 | 0.9575 | 1.9419 | 1.9419 | 2 | 2 |
| WCM185-TP2 | 19 | 54299338 | 59093645 | 0.0502 | 0.1651 | 1.1213 | 1.1213 | 1 | 1 |
| WCM185-TP2 | 2 | 41614 | 88999476 | −0.0867 | −0.0128 | 0.9912 | 0.9912 | 1 | 1 |
| WCM185-TP2 | 2 | 89034134 | 96648067 | 0.5495 | 0.7787 | 1.7968 | 1.6343 | 2 | 2 |
| WCM185-TP2 | 2 | 96652502 | 116601864 | −0.0298 | 0.0618 | 1.0438 | 1.0438 | 1 | 1 |
| WCM185-TP2 | 2 | 118572355 | 148705796 | −0.6733 | −0.8506 | 0.9906 | 0.1185 | 1 | 0 |
| WCM185-TP2 | 2 | 148710008 | 243037004 | −0.088 | −0.0146 | 0.99 | 0.99 | 1 | 1 |
| WCM185-TP2 | 20 | 68397 | 62934541 | −0.0708 | 0.0081 | 1.0057 | 1.0057 | 1 | 1 |
| WCM185-TP2 | 21 | 9590314 | 48084226 | −0.0859 | −0.0118 | 0.9919 | 0.9919 | 1 | 1 |
| WCM185-TP2 | 22 | 16159079 | 51193629 | −0.0844 | −0.0097 | 0.9933 | 0.9933 | 1 | 1 |
| WCM185-TP2 | 3 | 239472 | 197896724 | −0.0768 | 3.00E−04 | 1.0002 | 1.0002 | 1 | 1 |
| WCM185-TP2 | 4 | 53360 | 172735860 | −0.1044 | −0.0362 | 0.9752 | 0.9752 | 1 | 1 |
| WCM185-TP2 | 4 | 172761121 | 190878508 | −0.735 | −0.9487 | 0.9442 | 0.092 | 1 | 0 |
| WCM185-TP2 | 5 | 90714 | 840342 | −0.4971 | −0.5829 | 1.1957 | 0.1395 | 1 | 0 |
| WCM185-TP2 | 5 | 840575 | 53247452 | 0.0428 | 0.1556 | 1.1139 | 1.1139 | 1 | 1 |
| WCM185-TP2 | 5 | 53362913 | 55063722 | −0.6394 | −0.7978 | 0.9602 | 0.1903 | 1 | 0 |
| WCM185-TP2 | 5 | 55075867 | 58470295 | −2.0846 | −6.4512 | 0.0314 | −0.0085 | 0 | 0 |
| WCM185-TP2 | 5 | 58475470 | 80911313 | −0.5599 | −0.6764 | 1.1341 | 0.1173 | 1 | 0 |
| WCM185-TP2 | 5 | 80932452 | 86707209 | −0.0195 | 0.0752 | 1.1206 | 0.9864 | 1 | 1 |
| WCM185-TP2 | 5 | 86708647 | 102894868 | −0.5669 | −0.6871 | 1.1486 | 0.0936 | 1 | 0 |
| WCM185-TP2 | 5 | 102895139 | 131731248 | −0.0629 | 0.0185 | 1.0129 | 1.0129 | 1 | 1 |
| WCM185-TP2 | 5 | 131755651 | 133640258 | −0.7897 | −1.0378 | 0.972 | 0.0021 | 1 | 0 |
| WCM185-TP2 | 5 | 133642261 | 180687830 | −0.1084 | −0.0415 | 0.9716 | 0.9716 | 1 | 1 |
| WCM185-TP2 | 6 | 203428 | 69348629 | −0.1278 | −0.0672 | 0.9545 | 0.9545 | 1 | 1 |
| WCM185-TP2 | 6 | 69348824 | 126342407 | −0.7395 | −0.956 | 0.9278 | 0.1032 | 1 | 0 |
| WCM185-TP2 | 6 | 126349463 | 170892848 | −0.0985 | −0.0283 | 0.9806 | 0.9806 | 1 | 1 |
| WCM185-TP2 | 7 | 193254 | 56174230 | 0.3369 | 0.5233 | 1.5021 | 1.3724 | 2 | 1 |
| WCM185-TP2 | 7 | 56358786 | 65444845 | 0.9745 | 1.2699 | 2.5887 | 2.2342 | 3 | 2 |
| WCM185-TP2 | 7 | 65445312 | 158937161 | 0.3047 | 0.4839 | 1.4636 | 1.3333 | 1 | 1 |
| WCM185-TP2 | 8 | 190890 | 3226783 | −0.0869 | −0.013 | 0.991 | 0.991 | 1 | 1 |
| WCM185-TP2 | 8 | 3245054 | 34180923 | −0.7237 | −0.9305 | 0.9794 | 0.07 | 1 | 0 |
| WCM185-TP2 | 8 | 35383173 | 146279511 | 0.0235 | 0.1309 | 1.0949 | 1.0949 | 1 | 1 |
| WCM185-TP2 | 9 | 116944 | 141071646 | 0.1902 | 0.3423 | 1.2678 | 1.2678 | 1 | 1 |
| WCM185-TP2 | X | 2700189 | 154490238 | −0.0811 | −0.0053 | NA | NA | NA | NA |
| WCM185-TP3 | 1 | 17477 | 152009401 | −0.0876 | −0.0415 | 0.9717 | 0.9717 | 1 | 1 |
| WCM185-TP3 | 1 | 152052152 | 153177182 | 0.5987 | 0.8298 | 1.7775 | 1.7775 | 2 | 2 |
| WCM185-TP3 | 1 | 153190794 | 185277118 | −0.0937 | −0.0498 | 0.9661 | 0.9661 | 1 | 1 |
| WCM185-TP3 | 1 | 185277929 | 196758777 | −0.6518 | −0.8798 | 1.0257 | 0.0612 | 1 | 0 |
| WCM185-TP3 | 1 | 196762591 | 215793883 | −0.0399 | 0.0231 | 1.0161 | 1.0161 | 1 | 1 |
| WCM185-TP3 | 1 | 215794338 | 243289748 | −0.7222 | −0.9977 | 0.9192 | 0.0824 | 1 | 0 |
| WCM185-TP3 | 1 | 243292657 | 249213967 | 0.0289 | 0.115 | 1.0829 | 1.0829 | 1 | 1 |
| WCM185-TP3 | 10 | 225958 | 135439027 | −0.0549 | 0.0029 | 1.002 | 1.002 | 1 | 1 |
| WCM185-TP3 | 11 | 193112 | 48373815 | −0.0642 | −0.0097 | 0.9933 | 0.9933 | 1 | 1 |
| WCM185-TP3 | 11 | 48387206 | 55111013 | 0.4406 | 0.6384 | 1.6482 | 1.465 | 2 | 1 |
| WCM185-TP3 | 11 | 55111284 | 134605906 | −0.1099 | −0.0719 | 0.9514 | 0.9514 | 1 | 1 |
| WCM185-TP3 | 12 | 148454 | 133810883 | −0.1011 | −0.0599 | 0.9593 | 0.9593 | 1 | 1 |
| WCM185-TP3 | 13 | 19042005 | 115089411 | −0.073 | −0.0216 | 0.9851 | 0.9851 | 1 | 1 |
| WCM185-TP3 | 14 | 19110239 | 107283269 | −0.0942 | −0.0505 | 0.9656 | 0.9656 | 1 | 1 |
| WCM185-TP3 | 15 | 20083921 | 25277791 | 0.0849 | 0.1886 | 1.2097 | 1.0696 | 1 | 1 |
| WCM185-TP3 | 15 | 25287052 | 25599866 | 1.27 | 1.6036 | NA | NA | NA | NA |
| WCM185-TP3 | 15 | 25601049 | 102514189 | −0.0698 | −0.0174 | 0.988 | 0.988 | 1 | 1 |
| WCM185-TP3 | 16 | 67699 | 72188322 | −0.0924 | −0.048 | 0.9673 | 0.9673 | 1 | 1 |
| WCM185-TP3 | 16 | 72198706 | 90103742 | −0.7314 | −1.0133 | 0.9146 | 0.0763 | 1 | 0 |
| WCM185-TP3 | 16 | 90104154 | 90168680 | −0.0403 | 0.0225 | NA | NA | NA | NA |
| WCM185-TP3 | 17 | 131595 | 14110179 | −0.6757 | −0.9194 | 0.5692 | 0.4883 | 1 | 0 |
| WCM185-TP3 | 17 | 14139572 | 81052563 | −0.0995 | −0.0577 | 0.9608 | 0.9608 | 1 | 1 |
| WCM185-TP3 | 18 | 47801 | 27878947 | −0.0634 | −0.0086 | 0.9941 | 0.9941 | 1 | 1 |
| WCM185-TP3 | 18 | 28571229 | 78005279 | −0.3475 | −0.4082 | 0.7536 | 0.7536 | 1 | 1 |
| WCM185-TP3 | 19 | 105080 | 2515096 | −0.1197 | −0.0854 | 0.9968 | 0.8882 | 1 | 1 |
| WCM185-TP3 | 19 | 2520679 | 3985366 | −0.7292 | −1.0096 | 0.9114 | 0.082 | 1 | 0 |
| WCM185-TP3 | 19 | 4013004 | 8982204 | −0.0494 | 0.0103 | 1.0072 | 1.0072 | 1 | 1 |
| WCM185-TP3 | 19 | 8987030 | 9080462 | 0.6632 | 0.9068 | 1.8749 | 1.8749 | 2 | 2 |
| WCM185-TP3 | 19 | 9080646 | 11708361 | −0.1027 | −0.0621 | 0.9578 | 0.9578 | 1 | 1 |
| WCM185-TP3 | 19 | 11725438 | 12637528 | 0.6954 | 0.9449 | 1.9251 | 1.9251 | 2 | 2 |
| WCM185-TP3 | 19 | 12638056 | 19843710 | −0.1318 | −0.102 | 0.9317 | 0.9317 | 1 | 1 |
| WCM185-TP3 | 19 | 19905316 | 24310193 | 0.7049 | 0.9561 | 1.9401 | 1.9401 | 2 | 2 |
| WCM185-TP3 | 19 | 24310487 | 43015702 | −0.0391 | 0.0241 | 1.0168 | 1.0168 | 1 | 1 |
| WCM185-TP3 | 19 | 43016506 | 43780453 | 1.2447 | 1.5753 | 2.9801 | 2.9801 | 3 | 3 |
| WCM185-TP3 | 19 | 43857931 | 52887131 | −0.0951 | −0.0517 | 0.9648 | 0.9648 | 1 | 1 |
| WCM185-TP3 | 19 | 52887599 | 54265648 | 0.7614 | 1.0227 | 2.0317 | 2.0317 | 2 | 2 |
| WCM185-TP3 | 19 | 54290991 | 59093645 | 0.1217 | 0.2365 | 1.1782 | 1.1782 | 1 | 1 |
| WCM185-TP3 | 2 | 41474 | 88999476 | −0.0783 | −0.0288 | 0.9802 | 0.9802 | 1 | 1 |
| WCM185-TP3 | 2 | 89034028 | 96686913 | 0.5399 | 0.7591 | 1.7783 | 1.6067 | 2 | 2 |
| WCM185-TP3 | 2 | 96687324 | 116600767 | −0.031 | 0.035 | 1.0245 | 1.0245 | 1 | 1 |
| WCM185-TP3 | 2 | 116601631 | 148730283 | −0.6773 | −0.922 | 0.9647 | 0.0908 | 1 | 0 |
| WCM185-TP3 | 2 | 148731023 | 243037004 | −0.0723 | −0.0207 | 0.9858 | 0.9858 | 1 | 1 |
| WCM185-TP3 | 20 | 68311 | 62934623 | −0.0791 | −0.03 | 0.9794 | 0.9794 | 1 | 1 |
| WCM185-TP3 | 21 | 9590305 | 48084181 | −0.1015 | −0.0605 | 0.9589 | 0.9589 | 1 | 1 |
| WCM185-TP3 | 22 | 16159031 | 51193629 | −0.0723 | −0.0208 | 0.9857 | 0.9857 | 1 | 1 |
| WCM185-TP3 | 3 | 239472 | 197896711 | −0.064 | −0.0094 | 0.9935 | 0.9935 | 1 | 1 |
| WCM185-TP3 | 4 | 53360 | 172735749 | −0.0698 | −0.0173 | 0.9881 | 0.9881 | 1 | 1 |
| WCM185-TP3 | 4 | 172761121 | 190878508 | −0.7199 | −0.9938 | 0.9136 | 0.0908 | 1 | 0 |
| WCM185-TP3 | 5 | 90684 | 876309 | −0.5423 | −0.7037 | 1.1363 | 0.0917 | 1 | 0 |
| WCM185-TP3 | 5 | 878475 | 53362913 | 0.0051 | 0.0833 | 1.0594 | 1.0594 | 1 | 1 |
| WCM185-TP3 | 5 | 53389633 | 55063758 | −0.5704 | −0.7481 | 1.0782 | 0.1126 | 1 | 0 |
| WCM185-TP3 | 5 | 55075944 | 58571238 | −1.9417 | −6.0989 | 0.0354 | −0.0063 | 0 | 0 |
| WCM185-TP3 | 5 | 58652670 | 80809502 | −0.5967 | −0.7902 | 1.0524 | 0.1042 | 1 | 0 |
| WCM185-TP3 | 5 | 80911233 | 86707209 | 0.0206 | 0.1039 | 1.2272 | 0.9221 | 1 | 1 |
| WCM185-TP3 | 5 | 86708626 | 102614287 | −0.5685 | −0.7451 | 1.0725 | 0.1207 | 1 | 0 |
| WCM185-TP3 | 5 | 102886605 | 131731248 | −0.0608 | −0.0052 | 0.9964 | 0.9964 | 1 | 1 |
| WCM185-TP3 | 5 | 131755651 | 133640210 | −0.7609 | −1.0642 | 0.9022 | 0.0543 | 1 | 0 |
| WCM185-TP3 | 5 | 133642358 | 180687830 | −0.0928 | −0.0486 | 0.9669 | 0.9669 | 1 | 1 |
| WCM185-TP3 | 6 | 203479 | 67069279 | −0.1008 | −0.0595 | 0.9596 | 0.9596 | 1 | 1 |
| WCM185-TP3 | 6 | 67859534 | 126358837 | −0.7204 | −0.9945 | 0.9251 | 0.0787 | 1 | 0 |
| WCM185-TP3 | 6 | 126359931 | 170892848 | −0.0821 | −0.034 | 0.9767 | 0.9767 | 1 | 1 |
| WCM185-TP3 | 7 | 193222 | 56358942 | 0.3618 | 0.5411 | 1.5436 | 1.3666 | 2 | 1 |
| WCM185-TP3 | 7 | 56428074 | 65444853 | 0.9186 | 1.2054 | 2.6701 | 1.942 | 3 | 2 |
| WCM185-TP3 | 7 | 65445356 | 158937072 | 0.3441 | 0.5192 | 1.5492 | 1.3172 | 2 | 1 |
| WCM185-TP3 | 8 | 190850 | 3226761 | 0.0739 | 0.1742 | 1.1284 | 1.1284 | 1 | 1 |
| WCM185-TP3 | 8 | 3245054 | 35563496 | −0.7035 | −0.966 | 0.9349 | 0.0889 | 1 | 0 |
| WCM185-TP3 | 8 | 35579008 | 146279507 | 0.1025 | 0.2116 | 1.158 | 1.158 | 1 | 1 |
| WCM185-TP3 | 9 | 116815 | 141071646 | 0.1236 | 0.2391 | 1.1803 | 1.1803 | 1 | 1 |
| WCM185-TP3 | X | 2700194 | 154490194 | −0.1347 | −0.106 | NA | NA | NA | NA |
| WCM185-TP4 | 1 | 17465 | 152104264 | −0.0741 | NA | NA | NA | NA | NA |
| WCM185-TP4 | 1 | 152127392 | 153177196 | 0.6815 | NA | NA | NA | NA | NA |
| WCM185-TP4 | 1 | 153190787 | 249213961 | −0.0929 | NA | NA | NA | NA | NA |
| WCM185-TP4 | 10 | 226048 | 135439021 | −0.0702 | NA | NA | NA | NA | NA |
| WCM185-TP4 | 11 | 193112 | 134605906 | −0.0892 | NA | NA | NA | NA | NA |
| WCM185-TP4 | 12 | 148567 | 133810794 | −0.0965 | NA | NA | NA | NA | NA |
| WCM185-TP4 | 13 | 19042005 | 115089411 | −0.1112 | NA | NA | NA | NA | NA |
| WCM185-TP4 | 14 | 19110394 | 107283203 | −0.1017 | NA | NA | NA | NA | NA |
| WCM185-TP4 | 15 | 20083921 | 102514186 | −0.055 | NA | NA | NA | NA | NA |
| WCM185-TP4 | 16 | 67688 | 90168694 | −0.1067 | NA | NA | NA | NA | NA |
| WCM185-TP4 | 17 | 131595 | 81052609 | −0.0765 | NA | NA | NA | NA | NA |
| WCM185-TP4 | 18 | 47647 | 78005194 | −0.0911 | NA | NA | NA | NA | NA |
| WCM185-TP4 | 19 | 105010 | 11687372 | −0.0516 | NA | NA | NA | NA | NA |
| WCM185-TP4 | 19 | 11687630 | 12637487 | 0.602 | NA | NA | NA | NA | NA |
| WCM185-TP4 | 19 | 12637883 | 19824879 | −0.1208 | NA | NA | NA | NA | NA |
| WCM185-TP4 | 19 | 19825246 | 24310198 | 0.5843 | NA | NA | NA | NA | NA |
| WCM185-TP4 | 19 | 24310464 | 43023366 | −0.0764 | NA | NA | NA | NA | NA |
| WCM185-TP4 | 19 | 43025535 | 43780453 | 1.0716 | NA | NA | NA | NA | NA |
| WCM185-TP4 | 19 | 43857954 | 52919716 | −0.0833 | NA | NA | NA | NA | NA |
| WCM185-TP4 | 19 | 52919981 | 54265598 | 0.5703 | NA | NA | NA | NA | NA |
| WCM185-TP4 | 19 | 54290994 | 59093645 | 0.019 | NA | NA | NA | NA | NA |
| WCM185-TP4 | 2 | 41614 | 88913273 | −0.1012 | NA | NA | NA | NA | NA |
| WCM185-TP4 | 2 | 88998020 | 96795829 | 0.3926 | NA | NA | NA | NA | NA |
| WCM185-TP4 | 2 | 96798330 | 243037116 | −0.0737 | NA | NA | NA | NA | NA |
| WCM185-TP4 | 20 | 68312 | 62934572 | −0.0764 | NA | NA | NA | NA | NA |
| WCM185-TP4 | 21 | 9590316 | 48084136 | −0.067 | NA | NA | NA | NA | NA |
| WCM185-TP4 | 22 | 16159005 | 51193629 | −0.0512 | NA | NA | NA | NA | NA |
| WCM185-TP4 | 3 | 239495 | 197896656 | −0.0899 | NA | NA | NA | NA | NA |
| WCM185-TP4 | 4 | 53360 | 190878508 | −0.1049 | NA | NA | NA | NA | NA |
| WCM185-TP4 | 5 | 90750 | 180687830 | −0.0735 | NA | NA | NA | NA | NA |
| WCM185-TP4 | 6 | 203516 | 170892848 | −0.1308 | NA | NA | NA | NA | NA |
| WCM185-TP4 | 7 | 193254 | 158937158 | −0.0516 | NA | NA | NA | NA | NA |
| WCM185-TP4 | 8 | 190877 | 146279472 | −0.0906 | NA | NA | NA | NA | NA |
| WCM185-TP4 | 9 | 116800 | 141071646 | −0.0168 | NA | NA | NA | NA | NA |
| WCM185-TP4 | X | 2700156 | 154490196 | −0.0738 | NA | NA | NA | NA | NA |
| WCM185_X1_1 | 1 | 721450 | 212960903 | −0.0142 | 0.1025 | 1.1314 | 1.0159 | 1 | 1 |
| WCM185_X1_1 | 1 | 212964924 | 247109117 | −0.1682 | −1.543 | 0.6675 | 0.0189 | 1 | 0 |
| WCM185_X1_1 | 1 | 247130152 | 249212114 | −0.0766 | −0.3801 | 0.9342 | 0.6025 | 1 | 1 |
| WCM185_X1_1 | 10 | 225958 | 135440080 | −0.07 | −0.3221 | 0.8415 | 0.7582 | 1 | 1 |
| WCM185_X1_1 | 11 | 193112 | 134856562 | −0.0642 | −0.2729 | 0.8834 | 0.7719 | 1 | 1 |
| WCM185_X1_1 | 12 | 148673 | 133810713 | −0.0707 | −0.3279 | 0.8661 | 0.7273 | 1 | 1 |
| WCM185_X1_1 | 13 | 19600159 | 115089411 | −0.0098 | 0.1317 | 1.1825 | 1.0088 | 1 | 1 |
| WCM185_X1_1 | 14 | 19377733 | 107283068 | −0.0357 | −0.0485 | 1.0309 | 0.9029 | 1 | 1 |
| WCM185_X1_1 | 15 | 20170150 | 102515282 | 0.0282 | 0.3663 | 1.3634 | 1.2147 | 1 | 1 |
| WCM185_X1_1 | 16 | 67604 | 90233627 | 0.0276 | 0.3626 | 1.8429 | 0.7286 | 2 | 1 |
| WCM185_X1_1 | 17 | 131595 | 81083455 | 0.041 | 0.4382 | 1.4074 | 1.3025 | 1 | 1 |
| WCM185_X1_1 | 18 | 48965 | 78005219 | 0.0058 | 0.2319 | 1.2256 | 1.1232 | 1 | 1 |
| WCM185_X1_1 | 19 | 281424 | 59083923 | 0.0335 | 0.3965 | 1.7862 | 0.8464 | 2 | 1 |
| WCM185_X1_1 | 2 | 41620 | 242946597 | −0.0055 | 0.1602 | 1.1683 | 1.0666 | 1 | 1 |
| WCM185_X1_1 | 20 | 68363 | 62904845 | 0.0109 | 0.2634 | 1.237 | 1.1636 | 1 | 1 |
| WCM185_X1_1 | 21 | 9826203 | 48084226 | 0.008 | 0.2453 | 1.2953 | 1.0754 | 1 | 1 |
| WCM185_X1_1 | 22 | 17063440 | 51215130 | 0.0609 | 0.5453 | 1.5582 | 1.3605 | 2 | 1 |
| WCM185_X1_1 | 3 | 239436 | 197894720 | 0.0249 | 0.3473 | 1.3463 | 1.198 | 1 | 1 |
| WCM185_X1_1 | 4 | 59383 | 190426471 | −0.0926 | −0.5317 | 0.7532 | 0.6302 | 1 | 1 |
| WCM185_X1_1 | 5 | 140532 | 180687769 | −0.0935 | −0.5402 | 0.7833 | 0.5921 | 1 | 1 |
| WCM185_X1_1 | 6 | 203475 | 170893408 | −0.0679 | −0.3037 | 1.13 | 0.4903 | 1 | 0 |
| WCM185_X1_1 | 7 | 193222 | 158937400 | −0.0295 | −0.0035 | 1.5259 | 0.4692 | 2 | 0 |
| WCM185_X1_1 | 8 | 190907 | 146279416 | −0.0911 | −0.5168 | 0.7742 | 0.6237 | 1 | 1 |
| WCM185_X1_1 | 9 | 116819 | 139395118 | −0.0467 | −0.1316 | 0.9551 | 0.8705 | 1 | 1 |
| WCM185_X1_1 | 9 | 139396338 | 139397557 | −1.9589 | NA | NA | NA | NA | NA |
| WCM185_X1_1 | 9 | 139397678 | 141111414 | 0.0026 | 0.2117 | 1.4282 | 0.8879 | 1 | 1 |
| WCM185_X1_1 | X | 2700156 | 154509348 | −0.0852 | −0.4594 | NA | NA | NA | NA |
| WCM185_Z1_1 | 1 | 721450 | 157069273 | −0.0179 | −0.0135 | 0.9968 | 0.9845 | 1 | 1 |
| WCM185_Z1_1 | 1 | 157094682 | 157811477 | −0.7617 | −1.0083 | NA | NA | NA | NA |
| WCM185_Z1_1 | 1 | 157963503 | 185278606 | −0.0134 | −0.0079 | 0.9946 | 0.9946 | 1 | 1 |
| WCM185_Z1_1 | 1 | 185703968 | 196857331 | −0.7711 | −1.0221 | 1.0092 | −0.0245 | 1 | 0 |
| WCM185_Z1_1 | 1 | 196871717 | 215821013 | −0.0063 | 8.00E−04 | 1.0006 | 1.0006 | 1 | 1 |
| WCM185_Z1_1 | 1 | 215822033 | 243292679 | −0.7564 | −1.0006 | 1.0227 | −0.0231 | 1 | 0 |
| WCM185_Z1_1 | 1 | 243327940 | 249212238 | −0.0017 | 0.0066 | 1.0207 | 0.9885 | 1 | 1 |
| WCM185_Z1_1 | 10 | 226016 | 135440090 | −0.0128 | −0.0072 | 0.995 | 0.995 | 1 | 1 |
| WCM185_Z1_1 | 11 | 193112 | 134856562 | −0.0156 | −0.0106 | 0.9951 | 0.9902 | 1 | 1 |
| WCM185_Z1_1 | 12 | 148673 | 133810713 | −0.0125 | −0.0068 | 1.0027 | 0.988 | 1 | 1 |
| WCM185_Z1_1 | 13 | 19600147 | 115089411 | −0.0026 | 0.0054 | 1.0038 | 1.0038 | 1 | 1 |
| WCM185_Z1_1 | 14 | 19377711 | 36400963 | 0.0062 | 0.0163 | 1.0201 | 1.0026 | 1 | 1 |
| WCM185_Z1_1 | 14 | 36768359 | 38152053 | 0.6282 | 0.7509 | NA | NA | NA | NA |
| WCM185_Z1_1 | 14 | 38165967 | 107283183 | −0.0175 | −0.0129 | 0.9997 | 0.9825 | 1 | 1 |
| WCM185_Z1_1 | 15 | 20170150 | 102515266 | −0.0177 | −0.0132 | 0.997 | 0.9848 | 1 | 1 |
| WCM185_Z1_1 | 16 | 67604 | 72198737 | −0.0214 | −0.0177 | 0.9927 | 0.9829 | 1 | 1 |
| WCM185_Z1_1 | 16 | 72821209 | 90233626 | −0.7527 | −0.9951 | 1.0179 | −0.0144 | 1 | 0 |
| WCM185_Z1_1 | 17 | 131595 | 81083455 | −0.0278 | −0.0258 | 0.9908 | 0.9738 | 1 | 1 |
| WCM185_Z1_1 | 18 | 49039 | 78005214 | −0.012 | −0.0062 | 0.9957 | 0.9957 | 1 | 1 |
| WCM185_Z1_1 | 19 | 281506 | 2576047 | −0.0344 | −0.034 | 0.9973 | 0.9561 | 1 | 1 |
| WCM185_Z1_1 | 19 | 2642971 | 3984142 | −0.8066 | −1.0749 | 0.9457 | 0.0037 | 1 | 0 |
| WCM185_Z1_1 | 19 | 3985379 | 59083953 | −0.0504 | −0.0538 | 0.9706 | 0.9562 | 1 | 1 |
| WCM185_Z1_1 | 2 | 41613 | 118575215 | −0.0081 | −0.0014 | 1.0027 | 0.9953 | 1 | 1 |
| WCM185_Z1_1 | 2 | 118575296 | 148733472 | −0.7577 | −1.0024 | 1.0172 | −0.0188 | 1 | 0 |
| WCM185_Z1_1 | 2 | 149220220 | 242946604 | −0.007 | 0 | 1.0037 | 0.9963 | 1 | 1 |
| WCM185_Z1_1 | 20 | 68363 | 62904845 | −0.022 | −0.0186 | 0.9933 | 0.9811 | 1 | 1 |
| WCM185_Z1_1 | 21 | 9826207 | 48084214 | −0.0186 | −0.0144 | 0.995 | 0.9852 | 1 | 1 |
| WCM185_Z1_1 | 22 | 16287508 | 51215142 | −0.0212 | −0.0176 | 0.9977 | 0.9781 | 1 | 1 |
| WCM185_Z1_1 | 3 | 239431 | 197894713 | −0.0041 | 0.0036 | 1.0025 | 1.0025 | 1 | 1 |
| WCM185_Z1_1 | 4 | 59390 | 171010812 | −0.0048 | 0.0028 | 1.0019 | 1.0019 | 1 | 1 |
| WCM185_Z1_1 | 4 | 172735897 | 190426471 | −0.7622 | −1.009 | 0.995 | −0.0012 | 1 | 0 |
| WCM185_Z1_1 | 5 | 140532 | 878525 | −0.716 | −0.9415 | 1.038 | 0.0033 | 1 | 0 |
| WCM185_Z1_1 | 5 | 881237 | 53182522 | 0.0325 | 0.0486 | 1.0457 | 1.0229 | 1 | 1 |
| WCM185_Z1_1 | 5 | 53247347 | 55062786 | −0.7081 | −0.93 | 1.0185 | 0.0312 | 1 | 0 |
| WCM185_Z1_1 | 5 | 55063821 | 58476464 | −2.2118 | −4.9115 | NA | NA | NA | NA |
| WCM185_Z1_1 | 5 | 58481027 | 80932390 | −0.696 | −0.9126 | 1.0841 | −0.0217 | 1 | 0 |
| WCM185_Z1_1 | 5 | 81006528 | 86708587 | 3.00E−04 | 0.009 | NA | NA | NA | NA |
| WCM185_Z1_1 | 5 | 87486046 | 102886605 | −0.6006 | −0.7771 | 1.1562 | 0.0109 | 1 | 0 |
| WCM185_Z1_1 | 5 | 102887945 | 131729935 | 0.0515 | 0.0719 | 1.0704 | 1.0318 | 1 | 1 |
| WCM185_Z1_1 | 5 | 131755613 | 133561464 | −0.7379 | −0.9734 | 0.9824 | 0.0362 | 1 | 0 |
| WCM185_Z1_1 | 5 | 133622246 | 180687800 | −0.007 | 0 | 1 | 1 | 1 | 1 |
| WCM185_Z1_1 | 6 | 203561 | 66204848 | −0.0088 | −0.0023 | 1.0071 | 0.9898 | 1 | 1 |
| WCM185_Z1_1 | 6 | 66205150 | 126342320 | −0.7312 | −0.9637 | 1.0209 | 0.0046 | 1 | 0 |
| WCM185_Z1_1 | 6 | 126359850 | 170893424 | 0.0013 | 0.0102 | 1.0071 | 1.0071 | 1 | 1 |
| WCM185_Z1_1 | 7 | 193222 | 1883863 | 0.4683 | 0.5673 | 1.9846 | 0.9788 | 2 | 1 |
| WCM185_Z1_1 | 7 | 1886998 | 3197867 | 0.7351 | 0.8721 | 1.9521 | 1.7085 | 2 | 2 |
| WCM185_Z1_1 | 7 | 3205705 | 158937400 | 0.4808 | 0.5818 | 2.0057 | 0.9877 | 2 | 1 |
| WCM185_Z1_1 | 8 | 190907 | 3226824 | −0.0789 | −0.0894 | 0.9399 | 0.9399 | 1 | 1 |
| WCM185_Z1_1 | 8 | 3245054 | 33454987 | −0.7407 | −0.9775 | 1.0402 | −0.0245 | 1 | 0 |
| WCM185_Z1_1 | 8 | 35407004 | 146279514 | −0.0088 | −0.0023 | 1.0034 | 0.9935 | 1 | 1 |
| WCM185_Z1_1 | 9 | 116800 | 141111414 | 0.0042 | 0.0138 | 1.0096 | 1.0096 | 1 | 1 |
| WCM185_Z1_1 | X | 2700159 | 154509348 | −0.0322 | −0.0312 | NA | NA | NA | NA |
| WCM192 | 1 | 17462 | 249213961 | −0.0119 | NA | NA | NA | NA | NA |
| WCM192 | 10 | 225988 | 135438982 | −0.0803 | NA | NA | NA | NA | NA |
| WCM192 | 11 | 193112 | 134605954 | −0.0104 | NA | NA | NA | NA | NA |
| WCM192 | 12 | 148468 | 133810855 | −0.0394 | NA | NA | NA | NA | NA |
| WCM192 | 13 | 19041994 | 115089411 | −0.0543 | NA | NA | NA | NA | NA |
| WCM192 | 14 | 19110231 | 107283255 | −0.0189 | NA | NA | NA | NA | NA |
| WCM192 | 15 | 20083889 | 102514230 | −0.0056 | NA | NA | NA | NA | NA |
| WCM192 | 16 | 67570 | 90168646 | −0.0699 | NA | NA | NA | NA | NA |
| WCM192 | 17 | 131595 | 81052551 | −0.0552 | NA | NA | NA | NA | NA |
| WCM192 | 18 | 47797 | 78005210 | −0.0078 | NA | NA | NA | NA | NA |
| WCM192 | 19 | 105021 | 59093696 | 0.0652 | NA | NA | NA | NA | NA |
| WCM192 | 2 | 41675 | 101564763 | −0.0071 | NA | NA | NA | NA | NA |
| WCM192 | 2 | 101566016 | 179665386 | −0.1094 | NA | NA | NA | NA | NA |
| WCM192 | 2 | 179667035 | 243037013 | 0.0018 | NA | NA | NA | NA | NA |
| WCM192 | 20 | 68413 | 62934617 | 0.011 | NA | NA | NA | NA | NA |
| WCM192 | 21 | 9590312 | 48084214 | −0.0206 | NA | NA | NA | NA | NA |
| WCM192 | 22 | 16159043 | 51219089 | −0.0027 | NA | NA | NA | NA | NA |
| WCM192 | 3 | 239443 | 197896789 | −0.0152 | NA | NA | NA | NA | NA |
| WCM192 | 4 | 53359 | 190878479 | −0.0541 | NA | NA | NA | NA | NA |
| WCM192 | 5 | 90647 | 180687828 | −0.0122 | NA | NA | NA | NA | NA |
| WCM192 | 6 | 203364 | 170892873 | −0.0267 | NA | NA | NA | NA | NA |
| WCM192 | 7 | 193171 | 158937161 | 0.0029 | NA | NA | NA | NA | NA |
| WCM192 | 8 | 163527 | 146279545 | −0.0093 | NA | NA | NA | NA | NA |
| WCM192 | 9 | 14876 | 37917268 | −0.0985 | NA | NA | NA | NA | NA |
| WCM192 | 9 | 37919096 | 141107533 | 0.1113 | NA | NA | NA | NA | NA |
| WCM192 | X | 2700122 | 154509348 | −0.0294 | NA | NA | NA | NA | NA |
| WCM192_X1_1 | 1 | 721476 | 5940243 | 0.0791 | 0.0602 | 1.0613 | 1.0239 | 1 | 1 |
| WCM192_X1_1 | 1 | 5947386 | 21377390 | −0.7432 | −0.9982 | 0.998 | 0.0033 | 1 | 0 |
| WCM192_X1_1 | 1 | 21415705 | 43300813 | −0.0364 | −0.0792 | 0.9719 | 0.9212 | 1 | 1 |
| WCM192_X1_1 | 1 | 43304641 | 176132049 | 0.1147 | 0.1027 | 1.1594 | 0.9881 | 1 | 1 |
| WCM192_X1_1 | 1 | 176132997 | 207135760 | −0.0331 | −0.0751 | 0.9735 | 0.925 | 1 | 1 |
| WCM192_X1_1 | 1 | 207135828 | 229795037 | −0.4866 | −0.6485 | 0.9692 | 0.3067 | 1 | 0 |
| WCM192_X1_1 | 1 | 230314032 | 249212348 | 0.1161 | 0.1043 | 1.1032 | 1.0469 | 1 | 1 |
| WCM192_X1_1 | 10 | 226016 | 16918997 | −0.2555 | −0.3504 | 1.6301 | −0.0614 | 2 | 0 |
| WCM192_X1_1 | 10 | 16930461 | 47680417 | −0.5054 | −0.6733 | 0.9282 | 0.3259 | 1 | 0 |
| WCM192_X1_1 | 10 | 47681735 | 106160501 | −0.7457 | −1.0017 | 0.9776 | 0.0212 | 1 | 0 |
| WCM192_X1_1 | 10 | 106163516 | 128147658 | −0.5275 | −0.7028 | 1.2288 | 0 | 1 | 0 |
| WCM192_X1_1 | 10 | 128147884 | 135440108 | −0.7523 | −1.011 | 0.9477 | 0.0447 | 1 | 0 |
| WCM192_X1_1 | 11 | 193112 | 134856611 | −0.0624 | −0.1109 | 1.0108 | 0.8412 | 1 | 1 |
| WCM192_X1_1 | 12 | 148644 | 3982475 | −0.1042 | −0.162 | 0.8971 | 0.8905 | 1 | 1 |
| WCM192_X1_1 | 12 | 4383239 | 26878682 | −0.7849 | −1.0573 | 0.9726 | −0.0116 | 1 | 0 |
| WCM192_X1_1 | 12 | 26943134 | 38714877 | −0.0798 | −0.1321 | 0.9337 | 0.8913 | 1 | 1 |
| WCM192_X1_1 | 12 | 39047754 | 45695832 | 0.3887 | 0.4235 | 1.74 | 0.9423 | 2 | 1 |
| WCM192_X1_1 | 12 | 45740843 | 52754667 | −0.8235 | −1.1126 | 0.9229 | 0.002 | 1 | 0 |
| WCM192_X1_1 | 12 | 52755248 | 118693368 | −0.0326 | −0.0745 | 1.0466 | 0.8527 | 1 | 1 |
| WCM192_X1_1 | 12 | 118814556 | 120568537 | −0.7942 | −1.0705 | NA | NA | NA | NA |
| WCM192_X1_1 | 12 | 120569025 | 133810735 | −0.0874 | −0.1414 | 0.9222 | 0.8911 | 1 | 1 |
| WCM192_X1_1 | 13 | 19600365 | 30524481 | −0.8056 | −1.0868 | 0.9187 | 0.0229 | 1 | 0 |
| WCM192_X1_1 | 13 | 30782858 | 46137837 | −0.0399 | −0.0834 | 1.0105 | 0.8772 | 1 | 1 |
| WCM192_X1_1 | 13 | 46142549 | 61103114 | −0.6165 | −0.8227 | 1.1685 | −0.0377 | 1 | 0 |
| WCM192_X1_1 | 13 | 61109310 | 115089411 | 0.0899 | 0.0731 | 1.096 | 1.008 | 1 | 1 |
| WCM192_X1_1 | 14 | 19377726 | 107283060 | 0.1141 | 0.1019 | 1.1716 | 0.9748 | 1 | 1 |
| WCM192_X1_1 | 15 | 20170156 | 76452462 | 0.0845 | 0.0667 | 1.0598 | 1.0348 | 1 | 1 |
| WCM192_X1_1 | 15 | 76463463 | 102517667 | −0.6658 | −0.8902 | 1.0405 | 0.0386 | 1 | 0 |
| WCM192_X1_1 | 16 | 67604 | 46513110 | 0.1 | 0.0851 | 1.1582 | 0.9633 | 1 | 1 |
| WCM192_X1_1 | 16 | 46532030 | 67042966 | −0.6586 | −0.8803 | 1.0818 | 0.0046 | 1 | 0 |
| WCM192_X1_1 | 16 | 67063379 | 67261122 | −2.1219 | −3.9112 | NA | NA | NA | NA |
| WCM192_X1_1 | 16 | 67261739 | 90233642 | −0.6439 | −0.8602 | 1.0859 | 0.0158 | 1 | 0 |
| WCM192_X1_1 | 17 | 131595 | 4442235 | 0.5741 | 0.6352 | 2.1333 | 0.9731 | 2 | 1 |
| WCM192_X1_1 | 17 | 4442835 | 21826220 | 0.0907 | 0.074 | 1.0627 | 1.0426 | 1 | 1 |
| WCM192_X1_1 | 17 | 22023461 | 29326175 | −0.6436 | −0.8598 | 1.167 | −0.065 | 1 | 0 |
| WCM192_X1_1 | 17 | 29483072 | 61905539 | −0.2738 | −0.3735 | 1.5377 | 0.0062 | 2 | 0 |
| WCM192_X1_1 | 17 | 61906846 | 62084040 | −1.8644 | −3.0651 | NA | NA | NA | NA |
| WCM192_X1_1 | 17 | 62121480 | 81083507 | −0.2763 | −0.3767 | 1.5131 | 0.0272 | 2 | 0 |
| WCM192_X1_1 | 18 | 48266 | 33484792 | 0.1484 | 0.1427 | 1.1524 | 1.0555 | 1 | 1 |
| WCM192_X1_1 | 18 | 33552709 | 39535310 | −0.6827 | −0.9137 | NA | NA | NA | NA |
| WCM192_X1_1 | 18 | 39537555 | 78005203 | 0.1038 | 0.0897 | 1.087 | 1.0413 | 1 | 1 |
| WCM192_X1_1 | 19 | 281405 | 2123376 | −0.0623 | −0.1107 | 1.1331 | 0.7192 | 1 | 1 |
| WCM192_X1_1 | 19 | 2123863 | 30193878 | 0.1307 | 0.1217 | 1.2121 | 0.9639 | 1 | 1 |
| WCM192_X1_1 | 19 | 30199197 | 42603713 | 0.5773 | 0.6389 | 2.054 | 1.0602 | 2 | 1 |
| WCM192_X1_1 | 19 | 42603950 | 43430860 | −1.9977 | −3.46 | NA | NA | NA | NA |
| WCM192_X1_1 | 19 | 43433805 | 44739303 | 0.0435 | 0.0175 | 2.0722 | −0.0478 | 2 | 0 |
| WCM192_X1_1 | 19 | 44739399 | 58790713 | 0.5876 | 0.6505 | 2.0949 | 1.0444 | 2 | 1 |
| WCM192_X1_1 | 19 | 58790978 | 59083968 | −0.5777 | −0.77 | 1.1822 | −0.0094 | 1 | 0 |
| WCM192_X1_1 | 2 | 41613 | 99198091 | 0.1095 | 0.0965 | 1.0844 | 1.0539 | 1 | 1 |
| WCM192_X1_1 | 2 | 99204069 | 176957969 | −0.634 | −0.8465 | 1.1337 | −0.0214 | 1 | 0 |
| WCM192_X1_1 | 2 | 176958139 | 225761065 | 0.1254 | 0.1154 | 1.1168 | 1.0498 | 1 | 1 |
| WCM192_X1_1 | 2 | 225781443 | 229890400 | −0.6571 | −0.8783 | 1.0577 | 0.0303 | 1 | 0 |
| WCM192_X1_1 | 2 | 229890656 | 233470873 | 0.0835 | 0.0654 | 1.0789 | 1.0139 | 1 | 1 |
| WCM192_X1_1 | 2 | 233498564 | 237276918 | −0.3984 | −0.533 | 1.0217 | 0.3605 | 1 | 0 |
| WCM192_X1_1 | 2 | 237285764 | 238785909 | 0.027 | −0.0024 | NA | NA | NA | NA |
| WCM192_X1_1 | 2 | 238820208 | 239140843 | −0.6598 | −0.8819 | NA | NA | NA | NA |
| WCM192_X1_1 | 2 | 239147655 | 242946616 | 0.0253 | −0.0044 | 1.0498 | 0.9441 | 1 | 1 |
| WCM192_X1_1 | 20 | 68406 | 61945519 | 0.1058 | 0.092 | 1.1129 | 1.0189 | 1 | 1 |
| WCM192_X1_1 | 20 | 61946778 | 62904863 | 0.9753 | 1.0819 | 3.0083 | 1.2253 | 3 | 1 |
| WCM192_X1_1 | 21 | 9826235 | 48084214 | 0.06 | 0.0373 | 1.0459 | 1.0065 | 1 | 1 |
| WCM192_X1_1 | 22 | 16287446 | 39104945 | 0.1032 | 0.0889 | 1.0813 | 1.0458 | 1 | 1 |
| WCM192_X1_1 | 22 | 39111928 | 51214212 | −0.6473 | −0.8649 | 1.0565 | 0.0417 | 1 | 0 |
| WCM192_X1_1 | 3 | 239611 | 111870336 | 0.1012 | 0.0865 | 1.1606 | 0.963 | 1 | 1 |
| WCM192_X1_1 | 3 | 111870767 | 126452028 | −0.6348 | −0.8477 | 1.12 | −0.0086 | 1 | 0 |
| WCM192_X1_1 | 3 | 126571570 | 197894726 | 0.1165 | 0.1048 | 1.1611 | 0.9896 | 1 | 1 |
| WCM192_X1_1 | 4 | 59385 | 24556419 | 0.0543 | 0.0304 | 1.0507 | 0.9919 | 1 | 1 |
| WCM192_X1_1 | 4 | 24557921 | 28364204 | −0.8091 | −1.092 | NA | NA | NA | NA |
| WCM192_X1_1 | 4 | 28372367 | 78871010 | −0.0477 | −0.0929 | 0.9559 | 0.9193 | 1 | 1 |
| WCM192_X1_1 | 4 | 78979202 | 91832492 | −0.7392 | −0.9925 | 1.0084 | −0.0032 | 1 | 0 |
| WCM192_X1_1 | 4 | 91839399 | 115997565 | −0.3847 | −0.5153 | 1.4088 | −0.0095 | 1 | 0 |
| WCM192_X1_1 | 4 | 115997938 | 146092838 | −0.7908 | −1.0658 | 0.9883 | −0.0328 | 1 | 0 |
| WCM192_X1_1 | 4 | 146095863 | 185552305 | −0.4644 | −0.6192 | 0.9292 | 0.3729 | 1 | 0 |
| WCM192_X1_1 | 4 | 185552952 | 190426570 | −0.7457 | −1.0017 | 0.96 | 0.0388 | 1 | 0 |
| WCM192_X1_1 | 5 | 140532 | 35641648 | −0.0888 | −0.1432 | 0.9255 | 0.8855 | 1 | 1 |
| WCM192_X1_1 | 5 | 35646863 | 38528924 | −0.7444 | −0.9999 | NA | NA | NA | NA |
| WCM192_X1_1 | 5 | 38530749 | 90678841 | −0.0696 | −0.1196 | 0.9486 | 0.8923 | 1 | 1 |
| WCM192_X1_1 | 5 | 92921014 | 120022354 | −0.7711 | −1.0377 | 1.0019 | −0.0277 | 1 | 0 |
| WCM192_X1_1 | 5 | 121187718 | 124036846 | −0.1015 | −0.1587 | NA | NA | NA | NA |
| WCM192_X1_1 | 5 | 124044430 | 169017781 | 0.3658 | 0.397 | 1.6866 | 0.9471 | 2 | 1 |
| WCM192_X1_1 | 5 | 169018159 | 170149739 | −0.087 | −0.1409 | NA | NA | NA | NA |
| WCM192_X1_1 | 5 | 170159801 | 172196074 | 0.3866 | 0.421 | NA | NA | NA | NA |
| WCM192_X1_1 | 5 | 172196672 | 180687768 | −0.1307 | −0.1947 | 1.7851 | −0.0376 | 2 | 0 |
| WCM192_X1_1 | 6 | 203516 | 43970447 | −0.1006 | −0.1577 | 0.9141 | 0.8789 | 1 | 1 |
| WCM192_X1_1 | 6 | 43970827 | 46133178 | −0.838 | −1.1337 | 0.9862 | −0.0747 | 1 | 0 |
| WCM192_X1_1 | 6 | 46135347 | 170893450 | −0.0489 | −0.0944 | 1.0246 | 0.8488 | 1 | 1 |
| WCM192_X1_1 | 7 | 193265 | 158937046 | −0.0796 | −0.1319 | 1.0501 | 0.7752 | 1 | 1 |
| WCM192_X1_1 | 8 | 190906 | 146279527 | −0.0631 | −0.1118 | 1.0543 | 0.7966 | 1 | 1 |
| WCM192_X1_1 | 9 | 116854 | 28888879 | −0.7032 | −0.9421 | 1.0601 | −0.0191 | 1 | 0 |
| WCM192_X1_1 | 9 | 32384732 | 35243312 | −0.1626 | −0.2342 | 0.8881 | 0.8122 | 1 | 1 |
| WCM192_X1_1 | 9 | 35259018 | 35676179 | −0.9673 | −1.3251 | NA | NA | NA | NA |
| WCM192_X1_1 | 9 | 35677794 | 36223427 | −0.1048 | −0.1628 | NA | NA | NA | NA |
| WCM192_X1_1 | 9 | 36227271 | 36669402 | 0.2041 | 0.2085 | NA | NA | NA | NA |
| WCM192_X1_1 | 9 | 36671094 | 75773677 | 0 | −0.035 | 1.0209 | 0.9312 | 1 | 1 |
| WCM192_X1_1 | 9 | 75774304 | 141111414 | 0.4099 | 0.4479 | 1.7986 | 0.9295 | 2 | 1 |
| WCM192_X1_1 | X | 2700146 | 154509348 | −0.0213 | −0.0608 | NA | NA | NA | NA |
| WCM192_Z1_1 | 1 | 721415 | 5965831 | −0.0085 | 0.7126 | 2.247 | 1.0305 | 2 | 1 |
| WCM192_Z1_1 | 1 | 5967259 | 6184092 | −0.3487 | −0.0712 | NA | NA | NA | NA |
| WCM192_Z1_1 | 1 | 6184592 | 22174426 | −0.2962 | 0.0669 | 2.0896 | 0.0053 | 2 | 0 |
| WCM192_Z1_1 | 1 | 22174518 | 22181215 | −0.1615 | 0.3892 | NA | NA | NA | NA |
| WCM192_Z1_1 | 1 | 22181420 | 23645104 | −0.0653 | 0.5968 | 1.7545 | 1.2703 | 2 | 1 |
| WCM192_Z1_1 | 1 | 23650078 | 35454501 | −0.3573 | −0.0947 | 0.975 | 0.8979 | 1 | 1 |
| WCM192_Z1_1 | 1 | 35457747 | 35480334 | 0.297 | 1.269 | NA | NA | NA | NA |
| WCM192_Z1_1 | 1 | 35480677 | 204434438 | 0.0047 | 0.7388 | 2.276 | 1.0615 | 2 | 1 |
| WCM192_Z1_1 | 1 | 204435934 | 228582508 | 0.0767 | 0.8779 | 1.8863 | 1.7891 | 2 | 2 |
| WCM192_Z1_1 | 1 | 228582706 | 229794992 | 0.0719 | 0.8687 | NA | NA | NA | NA |
| WCM192_Z1_1 | 1 | 230314016 | 249212348 | 0.4619 | 1.5357 | 3.6425 | 2.1563 | 4 | 2 |
| WCM192_Z1_1 | 10 | 225952 | 135440105 | −0.2955 | 0.0688 | 2.1478 | −0.0501 | 2 | 0 |
| WCM192_Z1_1 | 11 | 193112 | 134856562 | −0.0202 | 0.6892 | 2.2093 | 1.0155 | 2 | 1 |
| WCM192_Z1_1 | 12 | 148674 | 26985684 | −0.2466 | 0.1906 | 2.217 | 0.0654 | 2 | 0 |
| WCM192_Z1_1 | 12 | 27059293 | 39064532 | 0.0043 | 0.7381 | 1.9451 | 1.3908 | 2 | 1 |
| WCM192_Z1_1 | 12 | 39071289 | 45664250 | 0.4755 | 1.5569 | 4.9181 | 0.9664 | 5 | 1 |
| WCM192_Z1_1 | 12 | 45686703 | 52756206 | −0.6149 | −0.9533 | 1.0771 | −0.0442 | 1 | 0 |
| WCM192_Z1_1 | 12 | 52756668 | 118828806 | 0.0024 | 0.7343 | 2.2336 | 1.0935 | 2 | 1 |
| WCM192_Z1_1 | 12 | 118829053 | 120569028 | −0.3255 | −0.0093 | NA | NA | NA | NA |
| WCM192_Z1_1 | 12 | 120569794 | 133810905 | −0.0176 | 0.6943 | 2.1251 | 1.1112 | 2 | 1 |
| WCM192_Z1_1 | 13 | 19600371 | 30521813 | −0.2807 | 0.1061 | 2.211 | −0.0583 | 2 | 0 |
| WCM192_Z1_1 | 13 | 30524459 | 46124385 | −0.0041 | 0.7213 | 2.1801 | 1.1173 | 2 | 1 |
| WCM192_Z1_1 | 13 | 46135505 | 61986918 | −0.2948 | 0.0705 | 2.1094 | −0.0092 | 2 | 0 |
| WCM192_Z1_1 | 13 | 61987202 | 115089411 | 0.0092 | 0.7477 | 2.2651 | 1.0931 | 2 | 1 |
| WCM192_Z1_1 | 14 | 19377701 | 107283027 | 0.1963 | 1.0959 | 2.1677 | 2.1073 | 2 | 2 |
| WCM192_Z1_1 | 15 | 20170091 | 76254301 | −0.3191 | 0.0076 | 1.1661 | 0.8444 | 1 | 1 |
| WCM192_Z1_1 | 15 | 76298096 | 102517678 | −0.6146 | −0.9518 | 1.0885 | −0.0545 | 1 | 0 |
| WCM192_Z1_1 | 16 | 67604 | 34682253 | 0.0601 | 0.8463 | 2.0997 | 1.4961 | 2 | 1 |
| WCM192_Z1_1 | 16 | 46508214 | 90233596 | −0.4965 | −0.5142 | 1.4394 | −0.039 | 1 | 0 |
| WCM192_Z1_1 | 17 | 131595 | 2203262 | 0.2434 | 1.1779 | 2.2937 | 2.2314 | 2 | 2 |
| WCM192_Z1_1 | 17 | 2203720 | 7623163 | 0.4039 | 1.4441 | 3.3956 | 2.0463 | 3 | 2 |
| WCM192_Z1_1 | 17 | 7626964 | 10318816 | 0.1709 | 1.0509 | 2.1533 | 1.9904 | 2 | 2 |
| WCM192_Z1_1 | 17 | 10319022 | 12823143 | 0.4783 | 1.5613 | 3.7303 | 2.1721 | 4 | 2 |
| WCM192_Z1_1 | 17 | 12832337 | 30211465 | 0.1069 | 0.9344 | 1.9714 | 1.8509 | 2 | 2 |
| WCM192_Z1_1 | 17 | 30213068 | 81083561 | −0.2941 | 0.0722 | 2.1781 | −0.0755 | 2 | 0 |
| WCM192_Z1_1 | 18 | 48224 | 1276033 | 0.1869 | 1.0793 | NA | NA | NA | NA |
| WCM192_Z1_1 | 18 | 1278621 | 55862847 | 0.2033 | 1.1082 | 2.2013 | 2.1104 | 2 | 2 |
| WCM192_Z1_1 | 18 | 55912692 | 78005215 | 0.0136 | 0.7563 | 2.2869 | 1.0914 | 2 | 1 |
| WCM192_Z1_1 | 19 | 281503 | 42599837 | 0.1846 | 1.0752 | 2.1406 | 2.0733 | 2 | 2 |
| WCM192_Z1_1 | 19 | 42600052 | 42719311 | −0.4647 | −0.4109 | NA | NA | NA | NA |
| WCM192_Z1_1 | 19 | 42720952 | 43414882 | −1.0721 | NA | NA | NA | NA | NA |
| WCM192_Z1_1 | 19 | 43420451 | 44721988 | −0.2974 | 0.0639 | 1.9982 | 0.0923 | 2 | 0 |
| WCM192_Z1_1 | 19 | 44732725 | 59083923 | 0.1789 | 1.0651 | 2.1295 | 2.0551 | 2 | 2 |
| WCM192_Z1_1 | 2 | 41623 | 676630 | 0.1784 | 1.0643 | NA | NA | NA | NA |
| WCM192_Z1_1 | 2 | 905302 | 44502788 | 0.0772 | 0.8788 | 2.1477 | 1.53 | 2 | 2 |
| WCM192_Z1_1 | 2 | 44502992 | 99198091 | 0.1957 | 1.095 | 2.1737 | 2.0984 | 2 | 2 |
| WCM192_Z1_1 | 2 | 99203972 | 176945234 | −0.2959 | 0.0677 | 2.0824 | 0.0137 | 2 | 0 |
| WCM192_Z1_1 | 2 | 176946914 | 189852834 | 0.0874 | 0.898 | 2.3842 | 1.3427 | 2 | 1 |
| WCM192_Z1_1 | 2 | 189853326 | 226378260 | 0.0105 | 0.7502 | 2.199 | 1.1651 | 2 | 1 |
| WCM192_Z1_1 | 2 | 226378359 | 228996775 | −0.3576 | −0.0955 | 2.1837 | −0.3119 | 2 | 0 |
| WCM192_Z1_1 | 2 | 229035210 | 238903428 | 0.1553 | 1.0228 | 2.0758 | 1.9879 | 2 | 2 |
| WCM192_Z1_1 | 2 | 238934045 | 239147660 | −0.3227 | −0.0018 | NA | NA | NA | NA |
| WCM192_Z1_1 | 2 | 239147783 | 242946597 | 0.1578 | 1.0274 | 2.097 | 1.9796 | 2 | 2 |
| WCM192_Z1_1 | 20 | 68396 | 61945435 | 0.1891 | 1.0833 | 2.1489 | 2.0889 | 2 | 2 |
| WCM192_Z1_1 | 20 | 61946779 | 62904866 | 0.7326 | 1.939 | 6.7261 | 0.9429 | 7 | 1 |
| WCM192_Z1_1 | 21 | 9826230 | 45649494 | −0.2638 | 0.1484 | 2.1797 | 0.0371 | 2 | 0 |
| WCM192_Z1_1 | 21 | 45649715 | 48084207 | −0.1533 | 0.4075 | 2.1783 | 0.4745 | 2 | 0 |
| WCM192_Z1_1 | 22 | 16287467 | 21576187 | −0.2908 | 0.0806 | 1.1112 | 1.0037 | 1 | 1 |
| WCM192_Z1_1 | 22 | 21799250 | 23629349 | −0.1609 | 0.3905 | 1.3962 | 1.2255 | 1 | 1 |
| WCM192_Z1_1 | 22 | 23630353 | 26040629 | 0.1583 | 1.0282 | 2.6452 | 1.4338 | 3 | 1 |
| WCM192_Z1_1 | 22 | 26057576 | 30639993 | −0.0369 | 0.6553 | 1.9661 | 1.1837 | 2 | 1 |
| WCM192_Z1_1 | 22 | 30640817 | 32914312 | 0.1609 | 1.0329 | 3.3002 | 0.792 | 3 | 1 |
| WCM192_Z1_1 | 22 | 32923962 | 39122435 | −0.0194 | 0.6907 | 1.9516 | 1.2765 | 2 | 1 |
| WCM192_Z1_1 | 22 | 39123282 | 42473441 | −0.3228 | −0.002 | 1.9455 | 0.0518 | 2 | 0 |
| WCM192_Z1_1 | 22 | 42473614 | 43459885 | 0.1703 | 1.0498 | 3.8953 | 0.2453 | 4 | 0 |
| WCM192_Z1_1 | 22 | 43460301 | 49042503 | −0.2918 | 0.0782 | 1.9935 | 0.1178 | 2 | 0 |
| WCM192_Z1_1 | 22 | 49089182 | 51219138 | −0.6144 | −0.9513 | 1.0694 | −0.0351 | 1 | 0 |
| WCM192_Z1_1 | 3 | 239443 | 29476406 | 0.0058 | 0.741 | 2.1536 | 1.1892 | 2 | 1 |
| WCM192_Z1_1 | 3 | 29529959 | 111860122 | −0.2774 | 0.1145 | 1.1179 | 1.0474 | 1 | 1 |
| WCM192_Z1_1 | 3 | 111870274 | 122496628 | −0.6291 | −1.0133 | 1.0846 | −0.0938 | 1 | 0 |
| WCM192_Z1_1 | 3 | 122512504 | 126348309 | −0.2986 | 0.0607 | 2.2519 | −0.1659 | 2 | 0 |
| WCM192_Z1_1 | 3 | 126349577 | 126633582 | −0.5023 | −0.5337 | NA | NA | NA | NA |
| WCM192_Z1_1 | 3 | 126645187 | 197894720 | 0.037 | 0.8021 | 2.3815 | 1.1057 | 2 | 1 |
| WCM192_Z1_1 | 4 | 59405 | 71275632 | −0.1253 | 0.4692 | 2.1956 | 0.5731 | 2 | 1 |
| WCM192_Z1_1 | 4 | 71339753 | 190426471 | −0.2682 | 0.1375 | 2.2582 | −0.0582 | 2 | 0 |
| WCM192_Z1_1 | 5 | 140532 | 35010172 | 0.2244 | 1.1451 | 2.3192 | 2.104 | 2 | 2 |
| WCM192_Z1_1 | 5 | 35013125 | 43613093 | −0.151 | 0.4126 | 1.8318 | 0.8304 | 2 | 1 |
| WCM192_Z1_1 | 5 | 43616071 | 90678832 | 0.2517 | 1.1923 | 2.3321 | 2.2381 | 2 | 2 |
| WCM192_Z1_1 | 5 | 92921008 | 119800199 | −0.2374 | 0.2127 | 2.3197 | −0.002 | 2 | 0 |
| WCM192_Z1_1 | 5 | 119816974 | 168690612 | 0.2077 | 1.116 | 2.1864 | 2.1485 | 2 | 2 |
| WCM192_Z1_1 | 5 | 169015496 | 169931634 | −0.2588 | 0.1608 | NA | NA | NA | NA |
| WCM192_Z1_1 | 5 | 170139864 | 172096791 | 0.1538 | 1.0202 | NA | NA | NA | NA |
| WCM192_Z1_1 | 5 | 172110465 | 180687768 | −0.3021 | 0.0517 | 2.0637 | 0.0093 | 2 | 0 |
| WCM192_Z1_1 | 6 | 203588 | 7569463 | −0.3269 | −0.0129 | 1.3714 | 0.6108 | 1 | 1 |
| WCM192_Z1_1 | 6 | 7570685 | 21065411 | −0.0421 | 0.6447 | 2.1481 | 0.9787 | 2 | 1 |
| WCM192_Z1_1 | 6 | 21198329 | 26431826 | 0.0769 | 0.8782 | 1.8936 | 1.7826 | 2 | 2 |
| WCM192_Z1_1 | 6 | 26444331 | 43028084 | 0.0366 | 0.8013 | 2.0193 | 1.4659 | 2 | 1 |
| WCM192_Z1_1 | 6 | 43029282 | 170893408 | 0.1143 | 0.9482 | 1.9721 | 1.8867 | 2 | 2 |
| WCM192_Z1_1 | 7 | 193254 | 76991907 | 0.0112 | 0.7517 | 2.1976 | 1.1699 | 2 | 1 |
| WCM192_Z1_1 | 7 | 77003457 | 158663881 | 0.2088 | 1.1179 | 2.2235 | 2.1171 | 2 | 2 |
| WCM192_Z1_1 | 7 | 158664253 | 158937072 | 0.1255 | 0.9687 | NA | NA | NA | NA |
| WCM192_Z1_1 | 8 | 190897 | 121475938 | 0.0274 | 0.7834 | 2.2476 | 1.1948 | 2 | 1 |
| WCM192_Z1_1 | 8 | 121483155 | 146279527 | 0.2078 | 1.1162 | 2.2438 | 2.0918 | 2 | 2 |
| WCM192_Z1_1 | 9 | 17079 | 312226 | −0.3785 | −0.1537 | NA | NA | NA | NA |
| WCM192_Z1_1 | 9 | 312494 | 27558505 | −0.2545 | 0.1713 | 2.276 | −0.0239 | 2 | 0 |
| WCM192_Z1_1 | 9 | 27560281 | 75450992 | −0.0394 | 0.6501 | 2.1967 | 0.9419 | 2 | 1 |
| WCM192_Z1_1 | 9 | 75516125 | 141111414 | 0.2121 | 1.1238 | 2.202 | 2.1563 | 2 | 2 |
| WCM192_Z1_1 | X | 2700122 | 154509348 | 0.1653 | 1.0409 | NA | NA | NA | NA |
| WCM195 | 1 | 14930 | 249213206 | 0.0343 | 0.3087 | 1.2386 | 1.2386 | 1 | 1 |
| WCM195 | 10 | 226118 | 135477898 | −0.0052 | −0.1609 | 0.8945 | 0.8945 | 1 | 1 |
| WCM195 | 11 | 193096 | 62259215 | 0.0269 | 0.2311 | 1.1737 | 1.1737 | 1 | 1 |
| WCM195 | 11 | 62283366 | 67375962 | −0.1063 | −4.1538 | 0.0562 | 0.0562 | 0 | 0 |
| WCM195 | 11 | 67376113 | 134605916 | 0.0092 | 0.0269 | 1.0188 | 1.0188 | 1 | 1 |
| WCM195 | 12 | 148452 | 133810752 | 0.0039 | −0.0398 | 0.9728 | 0.9728 | 1 | 1 |
| WCM195 | 13 | 19041994 | 115089411 | 0.0276 | 0.2389 | 1.1801 | 1.1801 | 1 | 1 |
| WCM195 | 14 | 19110349 | 107283156 | −0.0121 | −0.2585 | 0.836 | 0.836 | 1 | 1 |
| WCM195 | 15 | 20083896 | 102514189 | 0.0237 | 0.1955 | 1.1451 | 1.1451 | 1 | 1 |
| WCM195 | 16 | 74402 | 90260461 | −0.0087 | −0.2095 | 0.8648 | 0.8648 | 1 | 1 |
| WCM195 | 17 | 131595 | 81187934 | 0.003 | −0.0509 | 0.9654 | 0.9654 | 1 | 1 |
| WCM195 | 18 | 47818 | 78005201 | 0.0112 | 0.0512 | 1.0361 | 1.0361 | 1 | 1 |
| WCM195 | 19 | 105010 | 59093645 | −0.0447 | −0.8373 | 0.5597 | 0.5597 | 1 | 1 |
| WCM195 | 2 | 41620 | 243052790 | −0.0081 | −0.2007 | 0.8701 | 0.8701 | 1 | 1 |
| WCM195 | 20 | 68342 | 47887791 | 0.0426 | 0.3924 | 1.3125 | 1.3125 | 1 | 1 |
| WCM195 | 20 | 47887863 | 47897204 | −1.2965 | NA | NA | NA | NA | NA |
| WCM195 | 20 | 47905620 | 62934623 | 0.0111 | 0.0505 | 1.0356 | 1.0356 | 1 | 1 |
| WCM195 | 21 | 9590407 | 48084181 | −0.0102 | −0.2309 | 0.8521 | 0.8521 | 1 | 1 |
| WCM195 | 22 | 16159060 | 51219090 | 0.013 | 0.073 | 1.0519 | 1.0519 | 1 | 1 |
| WCM195 | 3 | 239398 | 197896740 | 0.0174 | 0.1253 | 1.0907 | 1.0907 | 1 | 1 |
| WCM195 | 4 | 53340 | 190903980 | −0.0023 | −0.121 | 0.9195 | 0.9195 | 1 | 1 |
| WCM195 | 5 | 90684 | 180687854 | 0.005 | −0.0251 | 0.9827 | 0.9827 | 1 | 1 |
| WCM195 | 6 | 203424 | 170892918 | −0.0123 | −0.262 | 0.8339 | 0.8339 | 1 | 1 |
| WCM195 | 7 | 193222 | 158937046 | 0.0149 | 0.0954 | 1.0683 | 1.0683 | 1 | 1 |
| WCM195 | 8 | 163476 | 146279511 | 0.0169 | 0.1189 | 1.0859 | 1.0859 | 1 | 1 |
| WCM195 | 9 | 14884 | 141107536 | 0.0452 | 0.4172 | 1.3353 | 1.3353 | 1 | 1 |
| WCM195 | X | 2700122 | 154493590 | −0.0701 | −1.5169 | NA | NA | NA | NA |
| WCM195_Z8_1 | 1 | 721476 | 37346317 | −0.1043 | 0.5985 | 1.9936 | 1.0347 | 2 | 1 |
| WCM195_Z8_1 | 1 | 37356679 | 38511660 | 0.6083 | 1.3305 | 3.0387 | 1.9911 | 3 | 2 |
| WCM195_Z8_1 | 1 | 38511716 | 52526164 | −0.1145 | 0.5879 | 2.018 | 0.9881 | 2 | 1 |
| WCM195_Z8_1 | 1 | 52530552 | 52769545 | 0.7188 | 1.4433 | NA | NA | NA | NA |
| WCM195_Z8_1 | 1 | 52800371 | 52981585 | −0.141 | 0.5605 | NA | NA | NA | NA |
| WCM195_Z8_1 | 1 | 52991442 | 59364929 | 0.6237 | 1.3463 | 3.1809 | 1.9041 | 3 | 2 |
| WCM195_Z8_1 | 1 | 59787414 | 66458662 | −0.1562 | 0.5447 | 1.9348 | 0.9827 | 2 | 1 |
| WCM195_Z8_1 | 1 | 66713293 | 85279851 | 0.5612 | 1.2824 | 2.8996 | 1.9652 | 3 | 2 |
| WCM195_Z8_1 | 1 | 85331194 | 120612037 | −0.1283 | 0.5736 | 1.9567 | 1.0198 | 2 | 1 |
| WCM195_Z8_1 | 1 | 143767833 | 150483355 | 0.5175 | 1.2377 | 3.5829 | 1.1335 | 4 | 1 |
| WCM195_Z8_1 | 1 | 150483644 | 150636176 | 0.6327 | 1.3554 | NA | NA | NA | NA |
| WCM195_Z8_1 | 1 | 150667278 | 151282710 | 1.1622 | 1.894 | NA | NA | NA | NA |
| WCM195_Z8_1 | 1 | 151288172 | 153751651 | 0.8041 | 1.5301 | 2.9027 | 2.8733 | 3 | 3 |
| WCM195_Z8_1 | 1 | 153751870 | 154933456 | 1.2292 | 1.9619 | 5.0646 | 2.7271 | 5 | 3 |
| WCM195_Z8_1 | 1 | 154936403 | 156646939 | 0.8293 | 1.5558 | 2.94 | 2.94 | 3 | 3 |
| WCM195_Z8_1 | 1 | 156669858 | 156843713 | 1.2478 | 1.9808 | NA | NA | NA | NA |
| WCM195_Z8_1 | 1 | 156844408 | 157494341 | 0.856 | 1.583 | NA | NA | NA | NA |
| WCM195_Z8_1 | 1 | 157497597 | 159558487 | 0.2292 | 0.9423 | 2.8494 | 0.9937 | 3 | 1 |
| WCM195_Z8_1 | 1 | 159683392 | 160681485 | 0.8128 | 1.539 | 2.9621 | 2.8496 | 3 | 3 |
| WCM195_Z8_1 | 1 | 160709117 | 190423813 | 0.2657 | 0.9797 | 2.9095 | 1.0346 | 3 | 1 |
| WCM195_Z8_1 | 1 | 192128425 | 192628621 | −1.357 | −0.7253 | NA | NA | NA | NA |
| WCM195_Z8_1 | 1 | 192778247 | 202574894 | 0.2434 | 0.9569 | 2.8523 | 1.03 | 3 | 1 |
| WCM195_Z8_1 | 1 | 202698267 | 203734472 | 0.797 | 1.5229 | NA | NA | NA | NA |
| WCM195_Z8_1 | 1 | 203734755 | 205902157 | 0.2562 | 0.9701 | 2.846 | 1.0718 | 3 | 1 |
| WCM195_Z8_1 | 1 | 205904837 | 207627762 | 0.4247 | 1.1428 | NA | NA | NA | NA |
| WCM195_Z8_1 | 1 | 207639933 | 211380649 | 0.8027 | 1.5286 | 3.035 | 2.7353 | 3 | 3 |
| WCM195_Z8_1 | 1 | 211381231 | 214567089 | 0.3056 | 1.0208 | NA | NA | NA | NA |
| WCM195_Z8_1 | 1 | 214568312 | 214781685 | 0.1093 | 0.8189 | NA | NA | NA | NA |
| WCM195_Z8_1 | 1 | 214782145 | 242612655 | −0.1547 | 0.5463 | 2.893 | 0.0276 | 3 | 0 |
| WCM195_Z8_1 | 1 | 242687390 | 245849969 | 0.3404 | 1.0564 | NA | NA | NA | NA |
| WCM195_Z8_1 | 1 | 245850070 | 247004281 | 0.8467 | 1.5734 | NA | NA | NA | NA |
| WCM195_Z8_1 | 1 | 247007137 | 249212348 | 0.2514 | 0.9651 | 2.8784 | 1.0259 | 3 | 1 |
| WCM195_Z8_1 | 10 | 225958 | 135440117 | −0.6631 | 0.0158 | 1.9789 | 0.0431 | 2 | 0 |
| WCM195_Z8_1 | 11 | 193112 | 17868254 | −0.1267 | 0.5753 | 1.9744 | 1.0056 | 2 | 1 |
| WCM195_Z8_1 | 11 | 17940305 | 18655750 | −0.662 | 0.0169 | 1.941 | 0.0825 | 2 | 0 |
| WCM195_Z8_1 | 11 | 18720435 | 66043540 | −0.1231 | 0.579 | 1.9361 | 1.0515 | 2 | 1 |
| WCM195_Z8_1 | 11 | 66045951 | 66083241 | −1.3126 | −0.6771 | NA | NA | NA | NA |
| WCM195_Z8_1 | 11 | 66083591 | 67821513 | −0.1229 | 0.5792 | 1.9549 | 1.0331 | 2 | 1 |
| WCM195_Z8_1 | 11 | 67829229 | 68216291 | −1.4833 | −0.8631 | NA | NA | NA | NA |
| WCM195_Z8_1 | 11 | 68305354 | 89951324 | −0.1155 | 0.5868 | 1.9046 | 1.0993 | 2 | 1 |
| WCM195_Z8_1 | 11 | 89955794 | 105948544 | −1.4994 | −0.8808 | 1.0238 | 0.0623 | 1 | 0 |
| WCM195_Z8_1 | 11 | 105950296 | 106856840 | −0.495 | 0.1921 | NA | NA | NA | NA |
| WCM195_Z8_1 | 11 | 107197707 | 108302472 | −1.4797 | −0.8592 | NA | NA | NA | NA |
| WCM195_Z8_1 | 11 | 108332229 | 110001831 | −0.1848 | 0.5151 | NA | NA | NA | NA |
| WCM195_Z8_1 | 11 | 110007543 | 122968504 | −1.4919 | −0.8725 | 1.0504 | 0.042 | 1 | 0 |
| WCM195_Z8_1 | 11 | 123065642 | 134856489 | −0.1255 | 0.5766 | 1.9545 | 1.0281 | 2 | 1 |
| WCM195_Z8_1 | 12 | 148675 | 8930211 | −0.1036 | 0.5992 | 2.0258 | 1.0038 | 2 | 1 |
| WCM195_Z8_1 | 12 | 8975308 | 16758746 | −1.4483 | −0.8248 | 1.0953 | 0.0338 | 1 | 0 |
| WCM195_Z8_1 | 12 | 17142102 | 133810703 | −0.1264 | 0.5755 | 1.9732 | 1.0073 | 2 | 1 |
| WCM195_Z8_1 | 13 | 19600515 | 23908034 | 1.068 | 1.7984 | 6.8679 | 0.0886 | 7 | 0 |
| WCM195_Z8_1 | 13 | 23908225 | 26978129 | 0.2276 | 0.9406 | 3.7888 | 0.0499 | 4 | 0 |
| WCM195_Z8_1 | 13 | 27216479 | 31543083 | −0.1677 | 0.5329 | NA | NA | NA | NA |
| WCM195_Z8_1 | 13 | 31711224 | 37014182 | 1.4277 | 2.1629 | 8.9091 | 0.047 | 9 | 0 |
| WCM195_Z8_1 | 13 | 37015314 | 42803258 | 0.274 | 0.9883 | 3.8758 | 0.092 | 4 | 0 |
| WCM195_Z8_1 | 13 | 42866613 | 43180895 | −1.5001 | −0.8815 | NA | NA | NA | NA |
| WCM195_Z8_1 | 13 | 43358204 | 43987026 | −0.1601 | 0.5407 | NA | NA | NA | NA |
| WCM195_Z8_1 | 13 | 44411561 | 47470930 | 1.4712 | 2.2069 | 9.1652 | 0.0688 | 9 | 0 |
| WCM195_Z8_1 | 13 | 48517540 | 50746944 | −1.4576 | −0.8349 | NA | NA | NA | NA |
| WCM195_Z8_1 | 13 | 51287361 | 53624814 | 1.3713 | 2.1058 | 8.5439 | 0.065 | 9 | 0 |
| WCM195_Z8_1 | 13 | 55748609 | 94680066 | −1.4364 | −0.8118 | 1.0158 | 0.1235 | 1 | 0 |
| WCM195_Z8_1 | 13 | 94938654 | 97639137 | 0.4282 | 1.1464 | NA | NA | NA | NA |
| WCM195_Z8_1 | 13 | 97639403 | 101944627 | 1.6339 | 2.3713 | 10.2808 | 0.0676 | 10 | 0 |
| WCM195_Z8_1 | 13 | 101997684 | 107823109 | 0.3701 | 1.0869 | 4.1746 | 0.0736 | 4 | 0 |
| WCM195_Z8_1 | 13 | 107863061 | 108955933 | −1.3898 | −0.761 | NA | NA | NA | NA |
| WCM195_Z8_1 | 13 | 109282197 | 111303432 | 1.12 | 1.8512 | 7.1809 | 0.0352 | 7 | 0 |
| WCM195_Z8_1 | 13 | 111315796 | 115089411 | 0.2795 | 0.994 | 3.9398 | 0.0434 | 4 | 0 |
| WCM195_Z8_1 | 14 | 19377703 | 23398410 | −0.6231 | 0.0578 | 1.9934 | 0.0884 | 2 | 0 |
| WCM195_Z8_1 | 14 | 23415772 | 23978555 | −0.153 | 0.5481 | NA | NA | NA | NA |
| WCM195_Z8_1 | 14 | 23991261 | 31058634 | −0.6211 | 0.0599 | 2.0192 | 0.0656 | 2 | 0 |
| WCM195_Z8_1 | 14 | 31059942 | 107283039 | −0.1022 | 0.6007 | 1.9934 | 1.0394 | 2 | 1 |
| WCM195_Z8_1 | 15 | 20170150 | 102517666 | −0.1543 | 0.5467 | 1.9344 | 0.9871 | 2 | 1 |
| WCM195_Z8_1 | 16 | 97436 | 90233601 | −0.1228 | 0.5794 | 1.9644 | 1.024 | 2 | 1 |
| WCM195_Z8_1 | 17 | 131595 | 18238908 | −0.6626 | 0.0163 | 1.9892 | 0.0336 | 2 | 0 |
| WCM195_Z8_1 | 17 | 18243508 | 22023436 | −0.6793 | −0.0013 | 1.4429 | 0.5553 | 1 | 1 |
| WCM195_Z8_1 | 17 | 25621438 | 81083567 | 0.344 | 1.0601 | 2.085 | 2.085 | 2 | 2 |
| WCM195_Z8_1 | 18 | 48241 | 78005226 | −0.1668 | 0.5337 | 1.9082 | 0.9871 | 2 | 1 |
| WCM195_Z8_1 | 19 | 281413 | 59083988 | −0.1366 | 0.565 | 1.9589 | 0.9999 | 2 | 1 |
| WCM195_Z8_1 | 2 | 41620 | 242842512 | −0.1508 | 0.5503 | 1.9347 | 0.9941 | 2 | 1 |
| WCM195_Z8_1 | 20 | 68396 | 21695425 | −0.6394 | 0.0406 | 1.0632 | 0.994 | 1 | 1 |
| WCM195_Z8_1 | 20 | 22562677 | 26084194 | 0.2591 | 0.973 | 2.9203 | 1.0055 | 3 | 1 |
| WCM195_Z8_1 | 20 | 26094564 | 43738633 | −0.1346 | 0.5671 | 1.8866 | 1.0766 | 2 | 1 |
| WCM195_Z8_1 | 20 | 43739119 | 45916015 | 0.2276 | 0.9406 | 1.9194 | 1.9194 | 2 | 2 |
| WCM195_Z8_1 | 20 | 45918931 | 62904847 | 0.5502 | 1.2711 | 2.8253 | 2.0016 | 3 | 2 |
| WCM195_Z8_1 | 21 | 9826230 | 32537369 | −1.4899 | −0.8704 | 1.0765 | 0.0175 | 1 | 0 |
| WCM195_Z8_1 | 21 | 32554954 | 35260482 | −0.0139 | 0.692 | 2.166 | 1.065 | 2 | 1 |
| WCM195_Z8_1 | 21 | 35276281 | 35774409 | −1.4363 | −0.8117 | NA | NA | NA | NA |
| WCM195_Z8_1 | 21 | 35791722 | 37586402 | −0.0291 | 0.6762 | 2.2086 | 0.9872 | 2 | 1 |
| WCM195_Z8_1 | 21 | 37586746 | 37711087 | −1.5961 | −0.9872 | NA | NA | NA | NA |
| WCM195_Z8_1 | 21 | 37713834 | 41741027 | −0.0333 | 0.6719 | 2.2317 | 0.9546 | 2 | 1 |
| WCM195_Z8_1 | 21 | 42064841 | 43557698 | −1.5137 | −0.8965 | 1.0091 | 0.0653 | 1 | 0 |
| WCM195_Z8_1 | 21 | 43621811 | 48084226 | −0.0299 | 0.6753 | 2.1974 | 0.9965 | 2 | 1 |
| WCM195_Z8_1 | 22 | 16287459 | 51219138 | −0.1534 | 0.5476 | 1.8963 | 1.0271 | 2 | 1 |
| WCM195_Z8_1 | 3 | 239436 | 197894726 | 0.2548 | 0.9686 | 1.9569 | 1.9569 | 2 | 2 |
| WCM195_Z8_1 | 4 | 59334 | 164061482 | −0.142 | 0.5595 | 1.9423 | 1.0051 | 2 | 1 |
| WCM195_Z8_1 | 4 | 164066938 | 186170237 | −0.6836 | −0.0059 | 1.9407 | 0.0512 | 2 | 0 |
| WCM195_Z8_1 | 4 | 186180113 | 186515084 | −0.1085 | 0.5941 | NA | NA | NA | NA |
| WCM195_Z8_1 | 4 | 186528433 | 187209696 | −0.6549 | 0.0244 | NA | NA | NA | NA |
| WCM195_Z8_1 | 4 | 187348233 | 190426502 | −0.1495 | 0.5517 | NA | NA | NA | NA |
| WCM195_Z8_1 | 5 | 140636 | 7897346 | 0.1731 | 0.8846 | 1.8785 | 1.814 | 2 | 2 |
| WCM195_Z8_1 | 5 | 7900145 | 10227732 | −0.7663 | −0.093 | NA | NA | NA | NA |
| WCM195_Z8_1 | 5 | 10235340 | 39377031 | 0.2093 | 0.9218 | 1.9646 | 1.8244 | 2 | 2 |
| WCM195_Z8_1 | 5 | 39377301 | 40681138 | −0.7287 | −0.0533 | NA | NA | NA | NA |
| WCM195_Z8_1 | 5 | 40681469 | 161580375 | 0.204 | 0.9164 | 1.8874 | 1.8874 | 2 | 2 |
| WCM195_Z8_1 | 5 | 162866353 | 162945299 | −0.6397 | 0.0404 | NA | NA | NA | NA |
| WCM195_Z8_1 | 5 | 166711885 | 180687769 | 0.2474 | 0.961 | 1.9607 | 1.9327 | 2 | 2 |
| WCM195_Z8_1 | 6 | 203509 | 16145325 | 0.2487 | 0.9623 | 1.9884 | 1.9084 | 2 | 2 |
| WCM195_Z8_1 | 6 | 16145435 | 66115161 | −0.1334 | 0.5684 | 1.9542 | 1.0114 | 2 | 1 |
| WCM195_Z8_1 | 6 | 66200549 | 74530266 | −1.5055 | −0.8874 | 1.0519 | 0.0292 | 1 | 0 |
| WCM195_Z8_1 | 6 | 74533342 | 81053500 | −0.1108 | 0.5918 | 1.7863 | 1.2279 | 2 | 1 |
| WCM195_Z8_1 | 6 | 82204799 | 89675731 | −1.4982 | −0.8795 | 1.0473 | 0.0398 | 1 | 0 |
| WCM195_Z8_1 | 6 | 89790737 | 124125396 | −0.1376 | 0.564 | 1.9346 | 1.0221 | 2 | 1 |
| WCM195_Z8_1 | 6 | 124604279 | 127440392 | −0.7201 | −0.0443 | NA | NA | NA | NA |
| WCM195_Z8_1 | 6 | 127469973 | 170893435 | −0.1301 | 0.5718 | 1.965 | 1.0077 | 2 | 1 |
| WCM195_Z8_1 | 7 | 193222 | 84727188 | −0.1373 | 0.5644 | 1.9473 | 1.0101 | 2 | 1 |
| WCM195_Z8_1 | 7 | 84751103 | 93633525 | −0.6633 | 0.0156 | 2.0031 | 0.0186 | 2 | 0 |
| WCM195_Z8_1 | 7 | 94024405 | 110603137 | −0.142 | 0.5595 | 1.9255 | 1.0219 | 2 | 1 |
| WCM195_Z8_1 | 7 | 110763015 | 117880031 | 0.3966 | 1.114 | NA | NA | NA | NA |
| WCM195_Z8_1 | 7 | 119914746 | 124533800 | −0.1446 | 0.5567 | 1.96 | 0.9819 | 2 | 1 |
| WCM195_Z8_1 | 7 | 126079195 | 133261358 | 0.2752 | 0.9895 | 2.01 | 1.9611 | 2 | 2 |
| WCM195_Z8_1 | 7 | 133261625 | 151049914 | 0.5347 | 1.2553 | 2.8703 | 1.9041 | 3 | 2 |
| WCM195_Z8_1 | 7 | 151052937 | 158937375 | −0.1228 | 0.5793 | 1.9226 | 1.0657 | 2 | 1 |
| WCM195_Z8_1 | 8 | 190907 | 49987715 | 0.2172 | 0.93 | 1.9248 | 1.8857 | 2 | 2 |
| WCM195_Z8_1 | 8 | 50449793 | 71646613 | −0.6824 | −0.0045 | 1.9467 | 0.0471 | 2 | 0 |
| WCM195_Z8_1 | 8 | 72111599 | 146279483 | 0.2268 | 0.9398 | 1.94 | 1.8966 | 2 | 2 |
| WCM195_Z8_1 | 9 | 17101 | 8733816 | 0.3001 | 1.015 | 2.0438 | 1.9981 | 2 | 2 |
| WCM195_Z8_1 | 9 | 12694159 | 16738401 | −0.6929 | −0.0156 | NA | NA | NA | NA |
| WCM195_Z8_1 | 9 | 17135137 | 17579270 | 0.3023 | 1.0173 | NA | NA | NA | NA |
| WCM195_Z8_1 | 9 | 17747061 | 18723029 | −0.7256 | −0.0501 | NA | NA | NA | NA |
| WCM195_Z8_1 | 9 | 18753396 | 19424700 | 0.1859 | 0.8978 | NA | NA | NA | NA |
| WCM195_Z8_1 | 9 | 19435061 | 21077820 | −0.6688 | 0.0098 | NA | NA | NA | NA |
| WCM195_Z8_1 | 9 | 21141078 | 21239884 | 0.2551 | 0.9689 | NA | NA | NA | NA |
| WCM195_Z8_1 | 9 | 21304913 | 27950658 | −0.6908 | −0.0134 | NA | NA | NA | NA |
| WCM195_Z8_1 | 9 | 28863629 | 35313921 | 0.2297 | 0.9428 | 2.023 | 1.8216 | 2 | 2 |
| WCM195_Z8_1 | 9 | 35364549 | 94712154 | 0.5474 | 1.2682 | 2.832 | 1.9853 | 3 | 2 |
| WCM195_Z8_1 | 9 | 94794652 | 100919921 | 0.2398 | 0.9532 | 2.8709 | 1.0014 | 3 | 1 |
| WCM195_Z8_1 | 9 | 100961805 | 139618094 | 0.5479 | 1.2688 | 2.843 | 1.9763 | 3 | 2 |
| WCM195_Z8_1 | 9 | 139618190 | 141111414 | 0.2889 | 1.0036 | 2.9926 | 1.0173 | 3 | 1 |
| WCM195_Z8_1 | X | 2707688 | 154509348 | 0.2178 | 0.9305 | NA | NA | NA | NA |
| WCM198 | 1 | 14930 | 249214028 | −0.1189 | 0.0233 | 1.0201 | 1.0124 | 1 | 1 |
| WCM198 | 10 | 225938 | 135438980 | −0.1225 | 0.0163 | 1.0114 | 1.0114 | 1 | 1 |
| WCM198 | 11 | 193097 | 134605831 | −0.1428 | −0.0229 | 0.9995 | 0.969 | 1 | 1 |
| WCM198 | 12 | 148388 | 7459291 | −0.1344 | −0.0066 | 1.0088 | 0.982 | 1 | 1 |
| WCM198 | 12 | 7459351 | 27919810 | −0.5581 | −0.9792 | 0.9893 | 0.0252 | 1 | 0 |
| WCM198 | 12 | 27924111 | 95927187 | −0.1461 | −0.0291 | 0.9971 | 0.9629 | 1 | 1 |
| WCM198 | 12 | 95927650 | 133810883 | 0.2223 | 0.6169 | 1.9979 | 1.0693 | 2 | 1 |
| WCM198 | 13 | 19042044 | 111536114 | −0.5838 | −1.0529 | 0.9457 | 0.0183 | 1 | 0 |
| WCM198 | 13 | 111545534 | 115089411 | −0.074 | 0.1077 | 2.1278 | 0.0272 | 2 | 0 |
| WCM198 | 14 | 19110394 | 20181525 | −0.3163 | −0.3804 | NA | NA | NA | NA |
| WCM198 | 14 | 20215815 | 21681123 | 0.4695 | 0.9948 | 1.9929 | 1.9929 | 2 | 2 |
| WCM198 | 14 | 21698574 | 31425358 | 0.8615 | 1.5376 | 3.6608 | 2.1456 | 4 | 2 |
| WCM198 | 14 | 31483953 | 31647448 | 0.448 | 0.9632 | NA | NA | NA | NA |
| WCM198 | 14 | 31674980 | 107283269 | −0.1286 | 0.0047 | 2.0068 | −4.00E−04 | 2 | 0 |
| WCM198 | 15 | 20083887 | 102506320 | −0.1306 | 8.00E−04 | 1.016 | 0.9852 | 1 | 1 |
| WCM198 | 16 | 67570 | 35136495 | −0.1496 | −0.0359 | 0.9963 | 0.9545 | 1 | 1 |
| WCM198 | 16 | 46507933 | 60393404 | −0.5599 | −0.9845 | 1.0125 | −0.0017 | 1 | 0 |
| WCM198 | 16 | 61687719 | 90260461 | −0.1284 | 0.005 | 1.0189 | 0.9881 | 1 | 1 |
| WCM198 | 17 | 131595 | 16675378 | −0.1722 | −0.0804 | 1.9195 | −0.0279 | 2 | 0 |
| WCM198 | 17 | 16676829 | 20209329 | 0.4577 | 0.9774 | 1.9747 | 1.9632 | 2 | 2 |
| WCM198 | 17 | 20217316 | 81187829 | 0.2143 | 0.6041 | 1.9647 | 1.0754 | 2 | 1 |
| WCM198 | 18 | 47593 | 15057262 | 0.6807 | 1.2943 | 3.8568 | 1.0483 | 4 | 1 |
| WCM198 | 18 | 15254367 | 24722788 | 0.2092 | 0.5958 | 1.9836 | 1.0391 | 2 | 1 |
| WCM198 | 18 | 24917001 | 78005123 | −0.5782 | −1.0367 | 0.9726 | 0.0022 | 1 | 0 |
| WCM198 | 19 | 105021 | 29699152 | 0.1906 | 0.5658 | 1.9297 | 1.0308 | 2 | 1 |
| WCM198 | 19 | 30017605 | 59021241 | 0.3956 | 0.8853 | 1.8472 | 1.8472 | 2 | 2 |
| WCM198 | 19 | 59021398 | 59093696 | 0.183 | 0.5534 | NA | NA | NA | NA |
| WCM198 | 2 | 41680 | 243052749 | −0.1543 | −0.0452 | 0.9691 | 0.9691 | 1 | 1 |
| WCM198 | 20 | 68337 | 62934623 | 0.1509 | 0.5008 | 1.9294 | 0.9006 | 2 | 1 |
| WCM198 | 21 | 9590314 | 39903442 | −0.1726 | −0.0811 | 0.9734 | 0.9173 | 1 | 1 |
| WCM198 | 21 | 39906086 | 42848452 | −0.5645 | −0.9975 | 1.1075 | −0.1057 | 1 | 0 |
| WCM198 | 21 | 42851135 | 48084198 | −0.084 | 0.089 | 1.0795 | 1.0478 | 1 | 1 |
| WCM198 | 22 | 16159046 | 51223730 | −0.1038 | 0.0518 | 1.0366 | 1.0366 | 1 | 1 |
| WCM198 | 3 | 239347 | 148460718 | −0.136 | −0.0096 | 0.9934 | 0.9934 | 1 | 1 |
| WCM198 | 3 | 148545681 | 197896818 | 0.1593 | 0.5146 | 1.8566 | 1.0006 | 2 | 1 |
| WCM198 | 4 | 53360 | 13604632 | −0.1965 | −0.1287 | 0.9385 | 0.8907 | 1 | 1 |
| WCM198 | 4 | 13604961 | 31144294 | −0.6118 | −1.1363 | 0.9166 | −0.0067 | 1 | 0 |
| WCM198 | 4 | 36069041 | 190904468 | −0.1587 | −0.0537 | 0.9635 | 0.9635 | 1 | 1 |
| WCM198 | 5 | 90751 | 45696101 | 0.6486 | 1.2499 | 3.7483 | 1.0082 | 4 | 1 |
| WCM198 | 5 | 49694853 | 116052172 | −0.5818 | −1.0472 | 0.9482 | 0.0196 | 1 | 0 |
| WCM198 | 5 | 118176549 | 129100561 | 0.1376 | 0.4788 | 1.6878 | 1.0994 | 2 | 1 |
| WCM198 | 5 | 129100796 | 134077219 | −0.6088 | −1.1272 | 0.9743 | −0.0586 | 1 | 0 |
| WCM198 | 5 | 134079712 | 139931766 | 0.1154 | 0.4417 | 1.6639 | 1.0525 | 2 | 1 |
| WCM198 | 5 | 139936747 | 140864810 | −0.096 | 0.0666 | NA | NA | NA | NA |
| WCM198 | 5 | 140865096 | 180687800 | 0.1506 | 0.5003 | 1.7184 | 1.1106 | 2 | 1 |
| WCM198 | 6 | 203475 | 170892848 | 0.1879 | 0.5614 | 1.9122 | 1.0391 | 2 | 1 |
| WCM198 | 7 | 193189 | 102114064 | −0.1437 | −0.0246 | 1.006 | 0.9602 | 1 | 1 |
| WCM198 | 7 | 102119351 | 104707175 | −0.5729 | −1.0215 | 1.0334 | −0.0482 | 1 | 0 |
| WCM198 | 7 | 104714103 | 158937046 | −0.1293 | 0.0033 | 1.0158 | 0.9888 | 1 | 1 |
| WCM198 | 8 | 163549 | 42958855 | −0.1247 | 0.012 | 1.9548 | 0.062 | 2 | 0 |
| WCM198 | 8 | 42977330 | 146279480 | 0.8366 | 1.5047 | 3.7138 | 1.9613 | 4 | 2 |
| WCM198 | 9 | 14811 | 334337 | 0.0558 | 0.3401 | NA | NA | NA | NA |
| WCM198 | 9 | 334430 | 16416376 | 0.457 | 0.9764 | 1.9676 | 1.9676 | 2 | 2 |
| WCM198 | 9 | 16416800 | 71436975 | 0.3467 | 0.8114 | 1.7951 | 1.7147 | 2 | 2 |
| WCM198 | 9 | 71437519 | 127934237 | 0.4684 | 0.9932 | 1.9906 | 1.9906 | 2 | 2 |
| WCM198 | 9 | 127951255 | 141107519 | 0.2265 | 0.6236 | 1.9892 | 1.0923 | 2 | 1 |
| WCM198 | X | 2700216 | 154509348 | −0.1884 | −0.1124 | NA | NA | NA | NA |
| WCM198_Z1_1 | 1 | 69590 | 249212550 | −0.2018 | 0.0379 | 1.0329 | 1.0204 | 1 | 1 |
| WCM198_Z1_1 | 10 | 225958 | 135440081 | −0.2608 | −0.0222 | 0.9927 | 0.9767 | 1 | 1 |
| WCM198_Z1_1 | 11 | 193112 | 134856536 | −0.2554 | −0.0167 | 1.046 | 0.931 | 1 | 1 |
| WCM198_Z1_1 | 12 | 148642 | 7361725 | −0.2285 | 0.0107 | 1.0167 | 0.9982 | 1 | 1 |
| WCM198_Z1_1 | 12 | 7362282 | 27916082 | −1.1518 | −0.939 | 1.0305 | 0.0127 | 1 | 0 |
| WCM198_Z1_1 | 12 | 27916224 | 33532808 | −0.2289 | 0.0104 | 1.0483 | 0.9661 | 1 | 1 |
| WCM198_Z1_1 | 12 | 33538103 | 38710819 | −1.1275 | −0.9138 | NA | NA | NA | NA |
| WCM198_Z1_1 | 12 | 38712108 | 96184039 | −0.2454 | −0.0066 | 1.0046 | 0.9863 | 1 | 1 |
| WCM198_Z1_1 | 12 | 96216930 | 133810896 | 0.3147 | 0.5631 | 1.9867 | 0.9682 | 2 | 1 |
| WCM198_Z1_1 | 13 | 19600159 | 111536111 | −1.1667 | −0.9545 | 1.0304 | 0.0016 | 1 | 0 |
| WCM198_Z1_1 | 13 | 111545487 | 115089411 | −0.2876 | −0.0496 | 1.9321 | 3.00E−04 | 2 | 0 |
| WCM198_Z1_1 | 14 | 19377727 | 21571367 | 0.6698 | 0.9225 | 1.8954 | 1.8954 | 2 | 2 |
| WCM198_Z1_1 | 14 | 21584732 | 31495229 | 1.2254 | 1.4831 | 3.6261 | 1.9648 | 4 | 2 |
| WCM198_Z1_1 | 14 | 31495393 | 31762562 | 0.8159 | 1.07 | NA | NA | NA | NA |
| WCM198_Z1_1 | 14 | 31762794 | 107283197 | −0.2385 | 5.00E−04 | 2.0007 | 0 | 2 | 0 |
| WCM198_Z1_1 | 15 | 20170150 | 74748972 | −0.2495 | −0.0107 | 1.0716 | 0.9136 | 1 | 1 |
| WCM198_Z1_1 | 15 | 74751168 | 75094783 | −1.176 | −0.9642 | NA | NA | NA | NA |
| WCM198_Z1_1 | 15 | 75105273 | 102506282 | −0.2735 | −0.0352 | 0.9939 | 0.958 | 1 | 1 |
| WCM198_Z1_1 | 16 | 67604 | 34682238 | −0.2747 | −0.0364 | 0.991 | 0.9591 | 1 | 1 |
| WCM198_Z1_1 | 16 | 46509877 | 60393311 | −1.2086 | −0.9981 | 1.0051 | −0.0037 | 1 | 0 |
| WCM198_Z1_1 | 16 | 61681688 | 90233642 | −0.2574 | −0.0188 | 1.0001 | 0.9739 | 1 | 1 |
| WCM198_Z1_1 | 17 | 131595 | 16594014 | −0.2678 | −0.0294 | 1.95 | 0.0096 | 2 | 0 |
| WCM198_Z1_1 | 17 | 16610800 | 20484011 | 0.6953 | 0.9482 | 1.9295 | 1.9295 | 2 | 2 |
| WCM198_Z1_1 | 17 | 20770002 | 22023445 | −0.4537 | −0.2194 | 0.9671 | 0.7508 | 1 | 1 |
| WCM198_Z1_1 | 17 | 25620983 | 81083455 | 0.3061 | 0.5543 | 1.9419 | 0.995 | 2 | 1 |
| WCM198_Z1_1 | 18 | 48996 | 15006107 | 1.0136 | 1.2695 | 3.8137 | 1.008 | 4 | 1 |
| WCM198_Z1_1 | 18 | 18531342 | 25543462 | 0.2969 | 0.545 | 2.002 | 0.9161 | 2 | 1 |
| WCM198_Z1_1 | 18 | 25565081 | 78005215 | −1.1679 | −0.9558 | 1.0333 | −0.0022 | 1 | 0 |
| WCM198_Z1_1 | 19 | 281467 | 24310774 | 0.2901 | 0.5381 | 1.9174 | 0.9868 | 2 | 1 |
| WCM198_Z1_1 | 19 | 29567134 | 59083970 | 0.683 | 0.9358 | 1.9129 | 1.9129 | 2 | 2 |
| WCM198_Z1_1 | 2 | 41620 | 242946607 | −0.2336 | 0.0055 | 1.0151 | 0.9925 | 1 | 1 |
| WCM198_Z1_1 | 20 | 68373 | 30686877 | 0.2226 | 0.4696 | 1.8794 | 0.8901 | 2 | 1 |
| WCM198_Z1_1 | 20 | 30689267 | 62904847 | 0.0042 | 0.2477 | 1.4772 | 0.8975 | 1 | 1 |
| WCM198_Z1_1 | 21 | 9826230 | 39818748 | −0.2404 | −0.0014 | 1.0224 | 0.9756 | 1 | 1 |
| WCM198_Z1_1 | 21 | 39822803 | 42851139 | −1.1656 | −0.9534 | NA | NA | NA | NA |
| WCM198_Z1_1 | 21 | 42852497 | 48084281 | −0.2866 | −0.0486 | 0.9817 | 0.9521 | 1 | 1 |
| WCM198_Z1_1 | 22 | 16287467 | 51219138 | −0.2553 | −0.0166 | 0.9976 | 0.9795 | 1 | 1 |
| WCM198_Z1_1 | 3 | 239347 | 148458977 | −0.2438 | −0.0049 | 1.0038 | 0.9895 | 1 | 1 |
| WCM198_Z1_1 | 3 | 148459322 | 197894720 | 0.2708 | 0.5185 | 1.8727 | 0.9923 | 2 | 1 |
| WCM198_Z1_1 | 4 | 59345 | 13629026 | −0.7032 | −0.4754 | 0.9774 | 0.4611 | 1 | 0 |
| WCM198_Z1_1 | 4 | 15003701 | 31144395 | −1.1946 | −0.9835 | 1.0108 | 6.00E−04 | 1 | 0 |
| WCM198_Z1_1 | 4 | 36069805 | 49052830 | −0.6145 | −0.3843 | 0.9667 | 0.5656 | 1 | 1 |
| WCM198_Z1_1 | 4 | 49063850 | 190426570 | −0.1445 | 0.0964 | 1.0789 | 1.0593 | 1 | 1 |
| WCM198_Z1_1 | 5 | 140526 | 45695823 | 0.9153 | 1.1704 | 3.5363 | 0.9653 | 4 | 1 |
| WCM198_Z1_1 | 5 | 49695734 | 115833034 | −1.1613 | −0.9489 | 1.0344 | 0.0017 | 1 | 0 |
| WCM198_Z1_1 | 5 | 115837961 | 129241266 | 0.1178 | 0.3632 | 1.575 | 0.9975 | 2 | 1 |
| WCM198_Z1_1 | 5 | 129243923 | 134077023 | −1.1583 | −0.9458 | 1.0155 | 0.0228 | 1 | 0 |
| WCM198_Z1_1 | 5 | 134079721 | 180687804 | 0.0602 | 0.3047 | 1.4976 | 0.9728 | 1 | 1 |
| WCM198_Z1_1 | 6 | 203699 | 170893408 | 0.2863 | 0.5342 | 1.9093 | 0.987 | 2 | 1 |
| WCM198_Z1_1 | 7 | 193254 | 102257116 | −0.2724 | −0.0341 | 1.0534 | 0.8999 | 1 | 1 |
| WCM198_Z1_1 | 7 | 102392141 | 104485841 | −1.1137 | −0.8995 | NA | NA | NA | NA |
| WCM198_Z1_1 | 7 | 104681423 | 158937018 | −0.2675 | −0.0291 | 0.993 | 0.967 | 1 | 1 |
| WCM198_Z1_1 | 8 | 190906 | 43052876 | −0.273 | −0.0347 | 1.9429 | 0.0095 | 2 | 0 |
| WCM198_Z1_1 | 8 | 43054597 | 146279423 | 1.2304 | 1.4881 | 3.7149 | 1.8953 | 4 | 2 |
| WCM198_Z1_1 | 9 | 17101 | 127996232 | 0.6781 | 0.9309 | 1.9065 | 1.9065 | 2 | 2 |
| WCM198_Z1_1 | 9 | 127999000 | 141111414 | 0.2579 | 0.5055 | 1.8559 | 0.9833 | 2 | 1 |
| WCM198_Z1_1 | X | 2700147 | 154509348 | −0.3112 | −0.0737 | NA | NA | NA | NA |
| WCM199 | 1 | 14907 | 249213458 | 0.059 | NA | NA | NA | NA | NA |
| WCM199 | 10 | 225912 | 135477892 | −0.0037 | NA | NA | NA | NA | NA |
| WCM199 | 11 | 193112 | 45935361 | 0.0727 | NA | NA | NA | NA | NA |
| WCM199 | 11 | 45935781 | 66193482 | −0.0318 | NA | NA | NA | NA | NA |
| WCM199 | 11 | 66193938 | 134605959 | 0.0436 | NA | NA | NA | NA | NA |
| WCM199 | 12 | 148427 | 133810769 | −0.0204 | NA | NA | NA | NA | NA |
| WCM199 | 13 | 19041965 | 115089411 | 0.0075 | NA | NA | NA | NA | NA |
| WCM199 | 14 | 19110194 | 107283169 | 0.029 | NA | NA | NA | NA | NA |
| WCM199 | 15 | 20083875 | 102514158 | 0.0064 | NA | NA | NA | NA | NA |
| WCM199 | 16 | 64401 | 90168697 | −0.0277 | NA | NA | NA | NA | NA |
| WCM199 | 17 | 131595 | 81052609 | 0.0044 | NA | NA | NA | NA | NA |
| WCM199 | 18 | 47686 | 78005206 | 0.0431 | NA | NA | NA | NA | NA |
| WCM199 | 19 | 105021 | 59093696 | −0.04 | NA | NA | NA | NA | NA |
| WCM199 | 2 | 41670 | 243037045 | −0.0049 | NA | NA | NA | NA | NA |
| WCM199 | 20 | 68337 | 62934572 | 0.0548 | NA | NA | NA | NA | NA |
| WCM199 | 21 | 9590311 | 48084201 | −0.0023 | NA | NA | NA | NA | NA |
| WCM199 | 22 | 16159043 | 51219090 | 0.0149 | NA | NA | NA | NA | NA |
| WCM199 | 3 | 239431 | 130114141 | 0.0209 | NA | NA | NA | NA | NA |
| WCM199 | 3 | 130116470 | 197659088 | 0.1088 | NA | NA | NA | NA | NA |
| WCM199 | 3 | 197665463 | 197896728 | −0.0426 | NA | NA | NA | NA | NA |
| WCM199 | 4 | 53345 | 190903980 | 0.0032 | NA | NA | NA | NA | NA |
| WCM199 | 5 | 90750 | 180687824 | 0.049 | NA | NA | NA | NA | NA |
| WCM199 | 6 | 203374 | 170892918 | −0.0067 | NA | NA | NA | NA | NA |
| WCM199 | 7 | 195702 | 158937046 | 0.0264 | NA | NA | NA | NA | NA |
| WCM199 | 8 | 163438 | 146279481 | 0.025 | NA | NA | NA | NA | NA |
| WCM199 | 9 | 14887 | 141107536 | 0.0155 | NA | NA | NA | NA | NA |
| WCM199 | X | 2700166 | 154493394 | −0.0569 | NA | NA | NA | NA | NA |
| WCM199_Z3_1 | 1 | 721415 | 29189597 | −0.328 | 0.0265 | 2.0315 | 0.0056 | 2 | 0 |
| WCM199_Z3_1 | 1 | 29314018 | 45671942 | 0.8136 | 1.3186 | 2.8936 | 2.0947 | 3 | 2 |
| WCM199_Z3_1 | 1 | 45792829 | 46531740 | −0.2834 | 0.0799 | NA | NA | NA | NA |
| WCM199_Z3_1 | 1 | 46532734 | 47280884 | 0.8377 | 1.3446 | NA | NA | NA | NA |
| WCM199_Z3_1 | 1 | 47282840 | 48267161 | −0.3505 | −6.00E−04 | NA | NA | NA | NA |
| WCM199_Z3_1 | 1 | 48459721 | 65349137 | 0.7819 | 1.2842 | 2.8447 | 2.0262 | 3 | 2 |
| WCM199_Z3_1 | 1 | 65523519 | 113256142 | 0.086 | 0.5111 | 1.8522 | 0.9981 | 2 | 1 |
| WCM199_Z3_1 | 1 | 113264930 | 192335065 | 0.775 | 1.2767 | 2.9431 | 1.9026 | 3 | 2 |
| WCM199_Z3_1 | 1 | 192545005 | 249212495 | 0.1142 | 0.5433 | 1.9343 | 0.9803 | 2 | 1 |
| WCM199_Z3_1 | 10 | 243934 | 128335169 | −0.377 | −0.0326 | 0.9883 | 0.967 | 1 | 1 |
| WCM199_Z3_1 | 10 | 128358921 | 129350888 | −1.1574 | −1.048 | NA | NA | NA | NA |
| WCM199_Z3_1 | 10 | 129534603 | 135440117 | −0.2961 | 0.0648 | 1.1597 | 0.9321 | 1 | 1 |
| WCM199_Z3_1 | 11 | 193112 | 17436875 | 0.131 | 0.5624 | 1.9846 | 0.9689 | 2 | 1 |
| WCM199_Z3_1 | 11 | 17438506 | 17498219 | −1.209 | −1.1221 | NA | NA | NA | NA |
| WCM199_Z3_1 | 11 | 17515924 | 78602285 | 0.128 | 0.559 | 1.9531 | 0.9933 | 2 | 1 |
| WCM199_Z3_1 | 11 | 78614351 | 83641490 | −0.4375 | −0.1061 | NA | NA | NA | NA |
| WCM199_Z3_1 | 11 | 83674042 | 134856523 | 0.0954 | 0.5219 | 1.9198 | 0.9518 | 2 | 1 |
| WCM199_Z3_1 | 12 | 148675 | 1042182 | −0.3521 | −0.0026 | NA | NA | NA | NA |
| WCM199_Z3_1 | 12 | 1137182 | 15067706 | −1.1087 | −0.9792 | 1.0214 | −0.0069 | 1 | 0 |
| WCM199_Z3_1 | 12 | 15068467 | 132467012 | −0.3731 | −0.0279 | 1.0212 | 0.9406 | 1 | 1 |
| WCM199_Z3_1 | 12 | 132471230 | 133810926 | −1.083 | −0.9433 | 1.047 | −0.0069 | 1 | 0 |
| WCM199_Z3_1 | 13 | 19600371 | 25077801 | 0.1173 | 0.5469 | 1.7008 | 1.221 | 2 | 1 |
| WCM199_Z3_1 | 13 | 25254891 | 26104202 | −0.3819 | −0.0385 | NA | NA | NA | NA |
| WCM199_Z3_1 | 13 | 26104750 | 46288145 | 0.0695 | 0.4922 | 1.7676 | 1.0457 | 2 | 1 |
| WCM199_Z3_1 | 13 | 46288408 | 48941658 | −0.4105 | −0.0732 | NA | NA | NA | NA |
| WCM199_Z3_1 | 13 | 48942680 | 49109832 | −2.5224 | −4.0008 | NA | NA | NA | NA |
| WCM199_Z3_1 | 13 | 49281121 | 50007525 | −1.0979 | −0.964 | NA | NA | NA | NA |
| WCM199_Z3_1 | 13 | 50018165 | 99112680 | −0.2986 | 0.0617 | 1.0724 | 1.015 | 1 | 1 |
| WCM199_Z3_1 | 13 | 99114053 | 115089411 | 0.184 | 0.6227 | 1.9985 | 1.0809 | 2 | 1 |
| WCM199_Z3_1 | 14 | 19377726 | 38183916 | 0.5339 | 1.0133 | 2.0186 | 2.0186 | 2 | 2 |
| WCM199_Z3_1 | 14 | 38194107 | 107283213 | 0.1443 | 0.5776 | 1.9879 | 0.9968 | 2 | 1 |
| WCM199_Z3_1 | 15 | 20170150 | 38990605 | −0.4074 | −0.0694 | 1.0792 | 0.8268 | 1 | 1 |
| WCM199_Z3_1 | 15 | 39544512 | 40576723 | −1.1531 | −1.0419 | NA | NA | NA | NA |
| WCM199_Z3_1 | 15 | 40581093 | 102517666 | −0.3785 | −0.0344 | 0.9906 | 0.9623 | 1 | 1 |
| WCM199_Z3_1 | 16 | 66943 | 2200038 | −0.989 | −0.8138 | 1.1128 | 0.025 | 1 | 0 |
| WCM199_Z3_1 | 16 | 2201144 | 68772307 | −0.3152 | 0.0418 | 1.0442 | 1.0146 | 1 | 1 |
| WCM199_Z3_1 | 16 | 68835730 | 90161609 | −1.0483 | −0.8952 | 1.0649 | 0.0105 | 1 | 0 |
| WCM199_Z3_1 | 17 | 131595 | 5487276 | 0.5462 | 1.0268 | 2.0666 | 2.0083 | 2 | 2 |
| WCM199_Z3_1 | 17 | 5984162 | 8812440 | −0.2997 | 0.0604 | 2.067 | 0.0185 | 2 | 0 |
| WCM199_Z3_1 | 17 | 8814786 | 12569512 | 0.4846 | 0.9589 | 1.9718 | 1.9159 | 2 | 2 |
| WCM199_Z3_1 | 17 | 12608461 | 20931877 | −0.345 | 0.0061 | 2.015 | −0.0065 | 2 | 0 |
| WCM199_Z3_1 | 17 | 20946104 | 21454632 | 0.0741 | 0.4975 | 1.491 | 1.3325 | 1 | 1 |
| WCM199_Z3_1 | 17 | 21730846 | 45895647 | −0.3315 | 0.0222 | 2.0113 | 0.0197 | 2 | 0 |
| WCM199_Z3_1 | 17 | 45895908 | 81083455 | 0.1439 | 0.5771 | 2.0314 | 0.9523 | 2 | 1 |
| WCM199_Z3_1 | 18 | 48234 | 52608292 | 0.4763 | 0.9497 | 1.94 | 1.9229 | 2 | 2 |
| WCM199_Z3_1 | 18 | 52609955 | 57569959 | −0.3668 | −0.0202 | 1.9781 | −0.0059 | 2 | 0 |
| WCM199_Z3_1 | 18 | 58038807 | 59828538 | 0.4107 | 0.877 | NA | NA | NA | NA |
| WCM199_Z3_1 | 18 | 59854838 | 78005210 | −0.3955 | −0.055 | 1.9035 | 0.0217 | 2 | 0 |
| WCM199_Z3_1 | 19 | 281508 | 59083929 | −0.3437 | 0.0075 | 1.119 | 0.8915 | 1 | 1 |
| WCM199_Z3_1 | 2 | 41623 | 242946604 | −0.3293 | 0.0249 | 1.0394 | 0.9954 | 1 | 1 |
| WCM199_Z3_1 | 20 | 68351 | 37005528 | 0.1456 | 0.5791 | 1.9151 | 1.0726 | 2 | 1 |
| WCM199_Z3_1 | 20 | 37053870 | 39742726 | −0.3441 | 0.0071 | NA | NA | NA | NA |
| WCM199_Z3_1 | 20 | 39744078 | 62904863 | 0.4794 | 0.9532 | 1.9361 | 1.9361 | 2 | 2 |
| WCM199_Z3_1 | 21 | 9826230 | 39817483 | −0.338 | 0.0144 | 1.1498 | 0.8703 | 1 | 1 |
| WCM199_Z3_1 | 21 | 39818741 | 42866297 | −1.0521 | −0.9003 | 1.0379 | 0.0336 | 1 | 0 |
| WCM199_Z3_1 | 21 | 42870050 | 48084211 | −0.2747 | 0.0902 | 1.0823 | 1.0468 | 1 | 1 |
| WCM199_Z3_1 | 22 | 16287437 | 26348385 | −0.3323 | 0.0213 | 1.0381 | 0.9917 | 1 | 1 |
| WCM199_Z3_1 | 22 | 26351317 | 29195959 | −1.217 | −1.1336 | 0.8891 | 0.0224 | 1 | 0 |
| WCM199_Z3_1 | 22 | 29279991 | 29976094 | −0.3175 | 0.0391 | 1.1051 | 0.9498 | 1 | 1 |
| WCM199_Z3_1 | 22 | 29977037 | 32924933 | −1.107 | −0.9768 | 1.0178 | −0.0016 | 1 | 0 |
| WCM199_Z3_1 | 22 | 32929841 | 51219138 | −0.3448 | 0.0063 | 1.0261 | 0.9826 | 1 | 1 |
| WCM199_Z3_1 | 3 | 239443 | 9920138 | −0.3282 | 0.0262 | 1.0587 | 0.978 | 1 | 1 |
| WCM199_Z3_1 | 3 | 9934740 | 10191495 | −1.0508 | −0.8986 | NA | NA | NA | NA |
| WCM199_Z3_1 | 3 | 10206701 | 26751779 | −0.3261 | 0.0287 | 1.0434 | 0.9968 | 1 | 1 |
| WCM199_Z3_1 | 3 | 27152767 | 35835317 | −1.0088 | −0.8408 | 1.0933 | 0.0234 | 1 | 0 |
| WCM199_Z3_1 | 3 | 36422135 | 47555052 | 0.1939 | 0.6339 | 1.9136 | 1.1899 | 2 | 1 |
| WCM199_Z3_1 | 3 | 47604152 | 53925815 | −0.3025 | 0.0571 | 2.105 | −0.0243 | 2 | 0 |
| WCM199_Z3_1 | 3 | 54157575 | 70014365 | 0.5144 | 0.9917 | 2.0039 | 1.9732 | 2 | 2 |
| WCM199_Z3_1 | 3 | 71007463 | 109321699 | 0.0519 | 0.472 | 1.8901 | 0.884 | 2 | 1 |
| WCM199_Z3_1 | 3 | 110611201 | 119962908 | −1.1064 | −0.9759 | 1.0177 | −8.00E−04 | 1 | 0 |
| WCM199_Z3_1 | 3 | 120050075 | 132440575 | 0.1458 | 0.5793 | 2.0446 | 0.9437 | 2 | 1 |
| WCM199_Z3_1 | 3 | 132440864 | 197894719 | 1.0075 | 1.5273 | 3.9414 | 1.8235 | 4 | 2 |
| WCM199_Z3_1 | 4 | 59448 | 190426570 | −0.3827 | −0.0395 | 1.0495 | 0.8964 | 1 | 1 |
| WCM199_Z3_1 | 5 | 140636 | 37247740 | 0.437 | 0.9062 | 1.9095 | 1.8388 | 2 | 2 |
| WCM199_Z3_1 | 5 | 37292124 | 39394368 | −0.4066 | −0.0685 | NA | NA | NA | NA |
| WCM199_Z3_1 | 5 | 40681118 | 180687776 | 0.462 | 0.9339 | 1.9252 | 1.8956 | 2 | 2 |
| WCM199_Z3_1 | 6 | 203510 | 108227912 | −0.3644 | −0.0174 | 0.9988 | 0.9773 | 1 | 1 |
| WCM199_Z3_1 | 6 | 108232558 | 109690101 | −1.0962 | −0.9617 | NA | NA | NA | NA |
| WCM199_Z3_1 | 6 | 109691656 | 160241520 | −0.3851 | −0.0423 | 0.9875 | 0.9546 | 1 | 1 |
| WCM199_Z3_1 | 6 | 160328065 | 160872045 | −1.0856 | −0.9469 | NA | NA | NA | NA |
| WCM199_Z3_1 | 6 | 160908460 | 170893411 | −0.3569 | −0.0083 | 1.0099 | 0.9787 | 1 | 1 |
| WCM199_Z3_1 | 7 | 193436 | 100608823 | 0.0846 | 0.5095 | 1.8893 | 0.9578 | 2 | 1 |
| WCM199_Z3_1 | 7 | 100609566 | 134962033 | −0.411 | −0.0738 | 1.9342 | −0.0339 | 2 | 0 |
| WCM199_Z3_1 | 7 | 135047620 | 158937416 | 0.4769 | 0.9504 | 1.9345 | 1.9303 | 2 | 2 |
| WCM199_Z3_1 | 8 | 190906 | 146279508 | 0.3432 | 0.8018 | 2.5322 | 0.9543 | 3 | 1 |
| WCM199_Z3_1 | 9 | 17086 | 3469160 | −0.4179 | −0.0823 | 0.9756 | 0.9135 | 1 | 1 |
| WCM199_Z3_1 | 9 | 3828281 | 7165343 | −1.1072 | −0.9771 | 1.0098 | 0.0062 | 1 | 0 |
| WCM199_Z3_1 | 9 | 7170006 | 22451906 | 0.1137 | 0.5427 | 1.9851 | 0.9284 | 2 | 1 |
| WCM199_Z3_1 | 9 | 23692600 | 28888941 | −1.1075 | −0.9775 | NA | NA | NA | NA |
| WCM199_Z3_1 | 9 | 32384732 | 141111414 | −0.3448 | 0.0063 | 1.0177 | 0.9911 | 1 | 1 |
| WCM199_Z3_1 | X | 2700139 | 51644748 | 0.2692 | 0.7188 | NA | NA | NA | NA |
| WCM199_Z3_1 | X | 52677392 | 154509348 | 0.5231 | 1.0014 | NA | NA | NA | NA |
| WCM1_T | 1 | 809178 | 898507 | 0.0422 | NA | NA | NA | NA | NA |
| WCM1_T | 1 | 898703 | 55224766 | −0.0485 | NA | NA | NA | NA | NA |
| WCM1_T | 1 | 55251822 | 145209427 | 0.0112 | NA | NA | NA | NA | NA |
| WCM1_T | 1 | 145295450 | 152060431 | −0.0956 | NA | NA | NA | NA | NA |
| WCM1_T | 1 | 152060670 | 153391648 | 0.1007 | NA | NA | NA | NA | NA |
| WCM1_T | 1 | 153409671 | 156764528 | −0.0628 | NA | NA | NA | NA | NA |
| WCM1_T | 1 | 156767081 | 249212465 | 0.0298 | NA | NA | NA | NA | NA |
| WCM1_T | 10 | 226039 | 69565336 | −0.03 | NA | NA | NA | NA | NA |
| WCM1_T | 10 | 69565561 | 70741319 | −0.2088 | NA | NA | NA | NA | NA |
| WCM1_T | 10 | 70742279 | 135440041 | −0.0198 | NA | NA | NA | NA | NA |
| WCM1_T | 11 | 193154 | 46455047 | 0.0459 | NA | NA | NA | NA | NA |
| WCM1_T | 11 | 46456582 | 48134313 | −0.0863 | NA | NA | NA | NA | NA |
| WCM1_T | 11 | 48134527 | 134257488 | 0.0189 | NA | NA | NA | NA | NA |
| WCM1_T | 12 | 247582 | 52470799 | −0.0068 | NA | NA | NA | NA | NA |
| WCM1_T | 12 | 52565153 | 53215782 | 0.183 | NA | NA | NA | NA | NA |
| WCM1_T | 12 | 53216113 | 56233259 | −0.0257 | NA | NA | NA | NA | NA |
| WCM1_T | 12 | 56233522 | 57181004 | −0.1007 | NA | NA | NA | NA | NA |
| WCM1_T | 12 | 57317623 | 118454721 | 0.0077 | NA | NA | NA | NA | NA |
| WCM1_T | 12 | 118456884 | 133779230 | −0.1263 | NA | NA | NA | NA | NA |
| WCM1_T | 13 | 19705463 | 115089411 | 0.0213 | NA | NA | NA | NA | NA |
| WCM1_T | 14 | 19553695 | 22294221 | −0.0208 | NA | NA | NA | NA | NA |
| WCM1_T | 14 | 22309448 | 23016560 | 0.5751 | NA | NA | NA | NA | NA |
| WCM1_T | 14 | 23016747 | 30066976 | 0.053 | NA | NA | NA | NA | NA |
| WCM1_T | 14 | 30068194 | 104474806 | −0.0118 | NA | NA | NA | NA | NA |
| WCM1_T | 14 | 104481083 | 107283156 | 0.0604 | NA | NA | NA | NA | NA |
| WCM1_T | 15 | 20170048 | 102359217 | −0.0357 | NA | NA | NA | NA | NA |
| WCM1_T | 16 | 97541 | 90162322 | −0.0449 | NA | NA | NA | NA | NA |
| WCM1_T | 17 | 131595 | 16340164 | −0.1042 | NA | NA | NA | NA | NA |
| WCM1_T | 17 | 16342582 | 80401890 | −0.0287 | NA | NA | NA | NA | NA |
| WCM1_T | 17 | 80402296 | 81052325 | −0.0743 | NA | NA | NA | NA | NA |
| WCM1_T | 18 | 47756 | 78005206 | 0.0118 | NA | NA | NA | NA | NA |
| WCM1_T | 19 | 156724 | 14063267 | −0.1231 | NA | NA | NA | NA | NA |
| WCM1_T | 19 | 14065176 | 16060772 | 0.0485 | NA | NA | NA | NA | NA |
| WCM1_T | 19 | 16178540 | 30314701 | −0.1454 | NA | NA | NA | NA | NA |
| WCM1_T | 19 | 30433534 | 36297389 | −0.0035 | NA | NA | NA | NA | NA |
| WCM1_T | 19 | 36297760 | 49794028 | −0.1336 | NA | NA | NA | NA | NA |
| WCM1_T | 19 | 49796617 | 59082605 | −0.0384 | NA | NA | NA | NA | NA |
| WCM1_T | 2 | 41595 | 64331975 | −0.0361 | NA | NA | NA | NA | NA |
| WCM1_T | 2 | 64335349 | 135476319 | 0.0214 | NA | NA | NA | NA | NA |
| WCM1_T | 2 | 135476550 | 136872444 | −0.0645 | NA | NA | NA | NA | NA |
| WCM1_T | 2 | 136872912 | 179399659 | 0.0664 | NA | NA | NA | NA | NA |
| WCM1_T | 2 | 179400105 | 179669281 | 0.1688 | NA | NA | NA | NA | NA |
| WCM1_T | 2 | 179701746 | 196771309 | 0.0713 | NA | NA | NA | NA | NA |
| WCM1_T | 2 | 196771736 | 242815461 | 0.0219 | NA | NA | NA | NA | NA |
| WCM1_T | 20 | 68427 | 62904819 | −0.006 | NA | NA | NA | NA | NA |
| WCM1_T | 21 | 9590301 | 48084202 | 0.0023 | NA | NA | NA | NA | NA |
| WCM1_T | 22 | 16255843 | 51216406 | −0.0194 | NA | NA | NA | NA | NA |
| WCM1_T | 3 | 361455 | 195622236 | 0.0367 | NA | NA | NA | NA | NA |
| WCM1_T | 3 | 195778899 | 197847360 | −0.0967 | NA | NA | NA | NA | NA |
| WCM1_T | 4 | 53400 | 140375549 | −0.0339 | NA | NA | NA | NA | NA |
| WCM1_T | 4 | 140393856 | 187630541 | 0.0449 | NA | NA | NA | NA | NA |
| WCM1_T | 4 | 187630910 | 190947560 | −0.2104 | NA | NA | NA | NA | NA |
| WCM1_T | 5 | 140657 | 180687808 | 0.0286 | NA | NA | NA | NA | NA |
| WCM1_T | 6 | 311934 | 170893507 | −0.0015 | NA | NA | NA | NA | NA |
| WCM1_T | 7 | 193546 | 6859561 | −0.053 | NA | NA | NA | NA | NA |
| WCM1_T | 7 | 7118608 | 38292948 | 0.0533 | NA | NA | NA | NA | NA |
| WCM1_T | 7 | 38295985 | 38388945 | 0.7638 | NA | NA | NA | NA | NA |
| WCM1_T | 7 | 38389276 | 55750390 | 0.0376 | NA | NA | NA | NA | NA |
| WCM1_T | 7 | 55752915 | 77648591 | −0.1074 | NA | NA | NA | NA | NA |
| WCM1_T | 7 | 77649052 | 97780714 | 0.0457 | NA | NA | NA | NA | NA |
| WCM1_T | 7 | 97784061 | 103123404 | −0.1042 | NA | NA | NA | NA | NA |
| WCM1_T | 7 | 103124075 | 142008695 | 0.0078 | NA | NA | NA | NA | NA |
| WCM1_T | 7 | 142008902 | 142612658 | 0.2814 | NA | NA | NA | NA | NA |
| WCM1_T | 7 | 142622780 | 158937400 | 0.0317 | NA | NA | NA | NA | NA |
| WCM1_T | 8 | 190908 | 48749976 | −0.0549 | NA | NA | NA | NA | NA |
| WCM1_T | 8 | 48751747 | 145806491 | 0.1368 | NA | NA | NA | NA | NA |
| WCM1_T | 8 | 145830968 | 146279396 | 0.0237 | NA | NA | NA | NA | NA |
| WCM1_T | 9 | 117703 | 127302393 | 0.0196 | NA | NA | NA | NA | NA |
| WCM1_T | 9 | 127306158 | 135278208 | −0.0468 | NA | NA | NA | NA | NA |
| WCM1_T | 9 | 135285650 | 141071165 | 0.0304 | NA | NA | NA | NA | NA |
| WCM1_T | X | 2700145 | 154509348 | 0.0292 | NA | NA | NA | NA | NA |
| WCM2 | 1 | 17468 | 249213964 | −0.0149 | −0.0673 | 0.9544 | 0.9544 | 1 | 1 |
| WCM2 | 10 | 225995 | 135439008 | 0.008 | 0.1202 | 1.0869 | 1.0869 | 1 | 1 |
| WCM2 | 11 | 193096 | 134605954 | −0.0334 | −0.2365 | 0.8488 | 0.8488 | 1 | 1 |
| WCM2 | 12 | 148637 | 133810703 | 0.0138 | 0.1651 | 1.1212 | 1.1212 | 1 | 1 |
| WCM2 | 13 | 19041994 | 115089411 | 0.0243 | 0.242 | 1.1826 | 1.1826 | 1 | 1 |
| WCM2 | 14 | 19110239 | 107283270 | 0.0288 | 0.2741 | 1.2093 | 1.2093 | 1 | 1 |
| WCM2 | 15 | 20083885 | 102506252 | 0.0515 | 0.4277 | 1.3451 | 1.3451 | 1 | 1 |
| WCM2 | 16 | 66881 | 304529 | −0.1161 | −1.3482 | NA | NA | NA | NA |
| WCM2 | 16 | 304633 | 28963480 | 0.0462 | 0.3926 | 1.3127 | 1.3127 | 1 | 1 |
| WCM2 | 16 | 28967440 | 90168680 | −0.1059 | −1.1597 | 0.4476 | 0.4476 | 0 | 0 |
| WCM2 | 17 | 131595 | 6900133 | −0.0746 | −0.6934 | 0.6184 | 0.6184 | 1 | 1 |
| WCM2 | 17 | 6901879 | 17394791 | −0.1948 | NA | −0.0172 | −0.0172 | 0 | 0 |
| WCM2 | 17 | 17397915 | 81187845 | −0.0347 | −0.2486 | 0.8417 | 0.8417 | 1 | 1 |
| WCM2 | 18 | 47790 | 78005274 | −0.1016 | −1.0865 | 0.4709 | 0.4709 | 0 | 0 |
| WCM2 | 19 | 105050 | 59093645 | −0.1026 | −1.1022 | 0.4658 | 0.4658 | 0 | 0 |
| WCM2 | 2 | 41474 | 243037003 | −0.0011 | 0.0484 | 1.0341 | 1.0341 | 1 | 1 |
| WCM2 | 20 | 68373 | 62959246 | 0.051 | 0.4241 | 1.3417 | 1.3417 | 1 | 1 |
| WCM2 | 21 | 9590393 | 48084163 | −0.0645 | −0.5678 | 0.6747 | 0.6747 | 1 | 1 |
| WCM2 | 22 | 16159016 | 51219090 | 0.0329 | 0.3027 | 1.2335 | 1.2335 | 1 | 1 |
| WCM2 | 3 | 239451 | 89448418 | −0.0241 | −0.1496 | 0.9015 | 0.9015 | 1 | 1 |
| WCM2 | 3 | 89456555 | 196050697 | 0.0462 | 0.3932 | 1.3133 | 1.3133 | 1 | 1 |
| WCM2 | 3 | 196050725 | 197896700 | −0.0424 | −0.326 | 0.7978 | 0.7978 | 1 | 1 |
| WCM2 | 4 | 53396 | 190878479 | −0.0297 | −0.2008 | 0.8701 | 0.8701 | 1 | 1 |
| WCM2 | 5 | 90647 | 52356692 | 0.1068 | 0.7521 | 1.6843 | 1.6843 | 2 | 2 |
| WCM2 | 5 | 52356935 | 145859690 | −0.047 | −0.3735 | 0.7719 | 0.7719 | 1 | 1 |
| WCM2 | 5 | 145862179 | 180687828 | 0.0626 | 0.4974 | 1.4117 | 1.4117 | 1 | 1 |
| WCM2 | 6 | 203588 | 170892848 | −0.0221 | −0.1311 | 0.9131 | 0.9131 | 1 | 1 |
| WCM2 | 7 | 193245 | 158937018 | 0.0865 | 0.6398 | 1.5581 | 1.5581 | 2 | 2 |
| WCM2 | 8 | 163474 | 57125511 | −0.0527 | −0.4336 | 0.7404 | 0.7404 | 1 | 1 |
| WCM2 | 8 | 57127251 | 125113312 | 0.1411 | 0.9265 | 1.9007 | 1.9007 | 2 | 2 |
| WCM2 | 8 | 125113606 | 146279489 | −0.0677 | −0.6068 | 0.6567 | 0.6567 | 1 | 1 |
| WCM2 | 9 | 14852 | 94895143 | −0.0716 | −0.6556 | 0.6348 | 0.6348 | 1 | 1 |
| WCM2 | 9 | 94896351 | 94899732 | −1.3712 | NA | NA | NA | NA | NA |
| WCM2 | 9 | 94900462 | 141071668 | −0.0289 | −0.1932 | 0.8747 | 0.8747 | 1 | 1 |
| WCM2 | X | 2700089 | 63005022 | 0.059 | 0.475 | NA | NA | NA | NA |
| WCM2 | X | 63406189 | 66942624 | 0.9706 | 3.1821 | NA | NA | NA | NA |
| WCM2 | X | 67268340 | 67742721 | 0.1623 | 1.0271 | NA | NA | NA | NA |
| WCM2 | X | 67751827 | 154493598 | −0.1414 | −1.9501 | NA | NA | NA | NA |
| WCM201 | 1 | 14930 | 249214018 | 0.0265 | 0.1054 | 1.0758 | 1.0758 | 1 | 1 |
| WCM201 | 10 | 225981 | 135438983 | 0.0241 | 0.0927 | 1.0663 | 1.0663 | 1 | 1 |
| WCM201 | 11 | 193096 | 62327458 | 0.0356 | 0.1521 | 1.1565 | 1.0659 | 1 | 1 |
| WCM201 | 11 | 62327793 | 66055525 | −0.1525 | −1.257 | 0.4831 | 0.3538 | 0 | 0 |
| WCM201 | 11 | 66061754 | 134605827 | 0.0317 | 0.1322 | 1.096 | 1.096 | 1 | 1 |
| WCM201 | 12 | 148452 | 133810842 | −0.002 | −0.0508 | 0.9654 | 0.9654 | 1 | 1 |
| WCM201 | 13 | 19042044 | 115089411 | 0.0191 | 0.066 | 1.0468 | 1.0468 | 1 | 1 |
| WCM201 | 14 | 19110203 | 107283269 | 3.00E−04 | −0.0374 | 0.9744 | 0.9744 | 1 | 1 |
| WCM201 | 15 | 20083857 | 102515250 | 0.0275 | 0.1102 | 1.0794 | 1.0794 | 1 | 1 |
| WCM201 | 16 | 64396 | 28975045 | −0.0098 | −0.096 | 0.9356 | 0.9356 | 1 | 1 |
| WCM201 | 16 | 28986442 | 31213839 | −0.2184 | −2.348 | 0.3912 | 0.0016 | 0 | 0 |
| WCM201 | 16 | 31228239 | 90260461 | −0.0108 | −0.1019 | 0.9318 | 0.9318 | 1 | 1 |
| WCM201 | 17 | 131595 | 81187931 | −0.0153 | −0.1288 | 0.9146 | 0.9146 | 1 | 1 |
| WCM201 | 18 | 47801 | 78005160 | 0.0294 | 0.1203 | 1.087 | 1.087 | 1 | 1 |
| WCM201 | 19 | 104937 | 59093645 | −0.0625 | −0.4371 | 0.7386 | 0.7386 | 1 | 1 |
| WCM201 | 2 | 41680 | 243052762 | 0.0174 | 0.0571 | 1.0403 | 1.0403 | 1 | 1 |
| WCM201 | 20 | 68351 | 62934623 | 0.0242 | 0.0932 | 1.0667 | 1.0667 | 1 | 1 |
| WCM201 | 21 | 9590369 | 48084196 | 0.0019 | −0.0288 | 0.9802 | 0.9802 | 1 | 1 |
| WCM201 | 22 | 16159079 | 51223730 | 0.0209 | 0.0756 | 1.0538 | 1.0538 | 1 | 1 |
| WCM201 | 3 | 239436 | 197896801 | 0.0229 | 0.086 | 1.0615 | 1.0615 | 1 | 1 |
| WCM201 | 4 | 53375 | 190878479 | 0.0257 | 0.1009 | 1.0724 | 1.0724 | 1 | 1 |
| WCM201 | 5 | 90576 | 180687854 | 0.0187 | 0.064 | 1.0454 | 1.0454 | 1 | 1 |
| WCM201 | 6 | 203545 | 170892854 | 0.0128 | 0.0317 | 1.0222 | 1.0222 | 1 | 1 |
| WCM201 | 7 | 193189 | 158937072 | 0.0232 | 0.088 | 1.0629 | 1.0629 | 1 | 1 |
| WCM201 | 8 | 163438 | 146279434 | 0.02 | 0.071 | 1.0504 | 1.0504 | 1 | 1 |
| WCM201 | 9 | 14856 | 141107533 | 0.0214 | 0.078 | 1.0555 | 1.0555 | 1 | 1 |
| WCM201 | X | 2700108 | 154493576 | −0.0995 | −0.7261 | NA | NA | NA | NA |
| WCM201_Z1_1 | 1 | 69603 | 110655683 | 0.0397 | 0.9464 | 1.9494 | 1.9046 | 2 | 2 |
| WCM201_Z1_1 | 1 | 110709771 | 116519280 | −0.6277 | 0.0071 | 2.0378 | −0.028 | 2 | 0 |
| WCM201_Z1_1 | 1 | 116534852 | 161683155 | 0.1189 | 1.0479 | 2.1174 | 2.0175 | 2 | 2 |
| WCM201_Z1_1 | 1 | 161692462 | 179851903 | −0.0437 | 0.8376 | 1.8482 | 1.726 | 2 | 2 |
| WCM201_Z1_1 | 1 | 179852118 | 193202135 | 0.1926 | 1.141 | 2.2689 | 2.1416 | 2 | 2 |
| WCM201_Z1_1 | 1 | 193205390 | 247875961 | −0.0832 | 0.7855 | 1.9249 | 1.5225 | 2 | 2 |
| WCM201_Z1_1 | 1 | 247886494 | 249212557 | 0.0673 | 0.9819 | 2.4115 | 1.5386 | 2 | 2 |
| WCM201_Z1_1 | 10 | 225952 | 27702540 | −0.1195 | 0.7372 | 1.7405 | 1.5933 | 2 | 2 |
| WCM201_Z1_1 | 10 | 27702725 | 44052903 | 0.0957 | 1.0183 | 2.0592 | 1.9919 | 2 | 2 |
| WCM201_Z1_1 | 10 | 44052986 | 63525749 | 0.2797 | 1.2495 | 2.7127 | 2.0424 | 3 | 2 |
| WCM201_Z1_1 | 10 | 63662089 | 81113483 | −0.1994 | 0.6292 | 1.8968 | 1.1966 | 2 | 1 |
| WCM201_Z1_1 | 10 | 81146052 | 116201540 | −0.6282 | 0.0063 | 1.053 | 0.9558 | 1 | 1 |
| WCM201_Z1_1 | 10 | 116203815 | 135440117 | −0.2472 | 0.5636 | 1.9371 | 1.0189 | 2 | 1 |
| WCM201_Z1_1 | 11 | 193106 | 134856626 | −0.0047 | 0.8887 | 1.8684 | 1.8346 | 2 | 2 |
| WCM201_Z1_1 | 12 | 148811 | 7475108 | −0.2755 | 0.5244 | 1.9125 | 0.9641 | 2 | 1 |
| WCM201_Z1_1 | 12 | 7476073 | 8673804 | 0.5377 | 1.5623 | NA | NA | NA | NA |
| WCM201_Z1_1 | 12 | 8687326 | 12044495 | −0.6153 | 0.0264 | 1.0896 | 0.9474 | 1 | 1 |
| WCM201_Z1_1 | 12 | 12232265 | 12673911 | 0.4248 | 1.4269 | NA | NA | NA | NA |
| WCM201_Z1_1 | 12 | 12765117 | 23757357 | −0.5153 | 0.1789 | 1.1896 | 1.0744 | 1 | 1 |
| WCM201_Z1_1 | 12 | 23793818 | 32520605 | 0.1849 | 1.1313 | 2.6972 | 1.684 | 3 | 2 |
| WCM201_Z1_1 | 12 | 32530498 | 54447966 | −0.0899 | 0.7765 | 1.7631 | 1.6629 | 2 | 2 |
| WCM201_Z1_1 | 12 | 54448661 | 55615327 | 0.4898 | 1.5051 | NA | NA | NA | NA |
| WCM201_Z1_1 | 12 | 55615403 | 56417527 | −0.0935 | 0.7717 | NA | NA | NA | NA |
| WCM201_Z1_1 | 12 | 56418797 | 77449736 | 0.2008 | 1.1512 | 2.2683 | 2.1737 | 2 | 2 |
| WCM201_Z1_1 | 12 | 77458341 | 82746972 | 1.1559 | 2.2732 | 7.3596 | 2.3084 | 7 | 2 |
| WCM201_Z1_1 | 12 | 82750772 | 102444064 | 0.0828 | 1.0018 | 2.1384 | 1.8665 | 2 | 2 |
| WCM201_Z1_1 | 12 | 102455047 | 107914392 | −0.2369 | 0.5779 | 2.064 | 0.9213 | 2 | 1 |
| WCM201_Z1_1 | 12 | 107937768 | 113269582 | −0.5837 | 0.0752 | 1.0972 | 1.0099 | 1 | 1 |
| WCM201_Z1_1 | 12 | 113275610 | 133810892 | −0.0114 | 0.8799 | 2.8254 | 0.8551 | 3 | 1 |
| WCM201_Z1_1 | 13 | 19600515 | 115089411 | −0.2886 | 0.5061 | 1.8366 | 1.0039 | 2 | 1 |
| WCM201_Z1_1 | 14 | 19377716 | 107283026 | −0.0262 | 0.8606 | 1.8372 | 1.7944 | 2 | 2 |
| WCM201_Z1_1 | 15 | 20170150 | 102514186 | −0.16 | 0.6828 | 1.7309 | 1.4795 | 2 | 1 |
| WCM201_Z1_1 | 16 | 67604 | 34267311 | 0.0145 | 0.9136 | 1.9009 | 1.8667 | 2 | 2 |
| WCM201_Z1_1 | 16 | 34274554 | 68293320 | −0.0795 | 0.7903 | 1.812 | 1.647 | 2 | 2 |
| WCM201_Z1_1 | 16 | 68308731 | 68397442 | −0.0013 | 0.8931 | NA | NA | NA | NA |
| WCM201_Z1_1 | 16 | 68397767 | 68598635 | −0.4374 | 0.294 | NA | NA | NA | NA |
| WCM201_Z1_1 | 16 | 68679291 | 72188271 | −0.3706 | 0.3904 | 1.4044 | 1.2171 | 1 | 1 |
| WCM201_Z1_1 | 16 | 72198345 | 90233626 | −0.5852 | 0.0728 | 1.4505 | 0.6531 | 1 | 1 |
| WCM201_Z1_1 | 17 | 131595 | 17171260 | −0.659 | −0.042 | 1.9361 | 0.0065 | 2 | 0 |
| WCM201_Z1_1 | 17 | 17174286 | 81083567 | 0.0069 | 0.9038 | 1.8953 | 1.8467 | 2 | 2 |
| WCM201_Z1_1 | 18 | 48203 | 28898294 | −0.0124 | 0.8787 | 1.9204 | 1.7571 | 2 | 2 |
| WCM201_Z1_1 | 18 | 28906543 | 40500703 | −0.2159 | 0.6067 | 2.0086 | 1.0369 | 2 | 1 |
| WCM201_Z1_1 | 18 | 40503597 | 53723814 | 0.0576 | 0.9694 | 1.9907 | 1.9252 | 2 | 2 |
| WCM201_Z1_1 | 18 | 54278293 | 56024461 | −0.5871 | 0.0699 | NA | NA | NA | NA |
| WCM201_Z1_1 | 18 | 56033398 | 78005197 | −0.0823 | 0.7867 | 1.8303 | 1.6199 | 2 | 2 |
| WCM201_Z1_1 | 19 | 281483 | 59083953 | −0.0071 | 0.8856 | 1.8644 | 1.8306 | 2 | 2 |
| WCM201_Z1_1 | 2 | 41623 | 3826444 | −0.1286 | 0.725 | 1.7482 | 1.5576 | 2 | 2 |
| WCM201_Z1_1 | 2 | 4135981 | 9143675 | −0.5537 | 0.121 | NA | NA | NA | NA |
| WCM201_Z1_1 | 2 | 9419481 | 12880905 | 0.0315 | 0.9357 | 1.987 | 1.8385 | 2 | 2 |
| WCM201_Z1_1 | 2 | 14774334 | 20251253 | −0.6531 | −0.0327 | NA | NA | NA | NA |
| WCM201_Z1_1 | 2 | 20402033 | 61597484 | 0.0114 | 0.9097 | 1.9128 | 1.8445 | 2 | 2 |
| WCM201_Z1_1 | 2 | 61605543 | 74458429 | −0.6128 | 0.0304 | 1.9984 | 0.0441 | 2 | 0 |
| WCM201_Z1_1 | 2 | 74459683 | 147596973 | 0.0121 | 0.9106 | 1.9262 | 1.8334 | 2 | 2 |
| WCM201_Z1_1 | 2 | 148653948 | 152460230 | −0.6968 | −0.1022 | 1.8251 | 0.0381 | 2 | 0 |
| WCM201_Z1_1 | 2 | 152466498 | 170558145 | 0.015 | 0.9143 | 1.9287 | 1.8406 | 2 | 2 |
| WCM201_Z1_1 | 2 | 170591627 | 171822576 | −0.669 | −0.0578 | NA | NA | NA | NA |
| WCM201_Z1_1 | 2 | 171850329 | 242946616 | 0.0115 | 0.9098 | 1.913 | 1.8447 | 2 | 2 |
| WCM201_Z1_1 | 20 | 68406 | 62904866 | −0.0296 | 0.8562 | 2.0214 | 1.5991 | 2 | 2 |
| WCM201_Z1_1 | 21 | 9826205 | 48084277 | −0.0112 | 0.8802 | 1.8743 | 1.807 | 2 | 2 |
| WCM201_Z1_1 | 22 | 17063485 | 51215138 | −0.1704 | 0.6686 | 1.6132 | 1.5658 | 2 | 2 |
| WCM201_Z1_1 | 3 | 239405 | 111439610 | 0.0526 | 0.9629 | 1.9493 | 1.9493 | 2 | 2 |
| WCM201_Z1_1 | 3 | 111451475 | 134670524 | −0.1543 | 0.6905 | 2.3581 | 0.8695 | 2 | 1 |
| WCM201_Z1_1 | 3 | 134670759 | 197894720 | 0.1972 | 1.1467 | 2.2641 | 2.1641 | 2 | 2 |
| WCM201_Z1_1 | 4 | 59327 | 190426459 | −0.0769 | 0.7938 | 1.752 | 1.7152 | 2 | 2 |
| WCM201_Z1_1 | 5 | 140632 | 52216256 | 0.0213 | 0.9225 | 1.9568 | 1.8339 | 2 | 2 |
| WCM201_Z1_1 | 5 | 52218705 | 79617319 | −0.2055 | 0.6209 | 1.9053 | 1.1702 | 2 | 1 |
| WCM201_Z1_1 | 5 | 79654971 | 180687776 | 0.0399 | 0.9466 | 1.9423 | 1.9124 | 2 | 2 |
| WCM201_Z1_1 | 6 | 203515 | 350829 | −0.3525 | 0.4162 | NA | NA | NA | NA |
| WCM201_Z1_1 | 6 | 367481 | 16328258 | −0.3344 | 0.4419 | 1.5505 | 1.1663 | 2 | 1 |
| WCM201_Z1_1 | 6 | 16328394 | 170892637 | −0.0499 | 0.8295 | 1.8004 | 1.7536 | 2 | 2 |
| WCM201_Z1_1 | 7 | 193451 | 158937135 | 0.0707 | 0.9862 | 1.9962 | 1.9657 | 2 | 2 |
| WCM201_Z1_1 | 8 | 190907 | 41399565 | −0.7811 | −0.2398 | 1.6573 | 0.0365 | 2 | 0 |
| WCM201_Z1_1 | 8 | 41456684 | 146279545 | 0.1003 | 1.0241 | 2.0596 | 2.0077 | 2 | 2 |
| WCM201_Z1_1 | 9 | 17075 | 37955919 | −0.2129 | 0.6108 | 1.9011 | 1.153 | 2 | 1 |
| WCM201_Z1_1 | 9 | 37974678 | 105767903 | 0.0162 | 0.9159 | 1.9308 | 1.8426 | 2 | 2 |
| WCM201_Z1_1 | 9 | 106857726 | 141111414 | 0.2791 | 1.2487 | 2.87 | 1.8826 | 3 | 2 |
| WCM201_Z1_1 | X | 2707687 | 10427687 | −0.1191 | 0.7377 | NA | NA | NA | NA |
| WCM201_Z1_1 | X | 10437702 | 11139089 | 0.7532 | 1.8155 | NA | NA | NA | NA |
| WCM201_Z1_1 | X | 11139926 | 27839452 | 0.2933 | 1.2662 | NA | NA | NA | NA |
| WCM201_Z1_1 | X | 27839708 | 57936469 | 0.5808 | 1.6134 | NA | NA | NA | NA |
| WCM201_Z1_1 | X | 62898360 | 73095467 | 0.074 | 0.9904 | NA | NA | NA | NA |
| WCM201_Z1_1 | X | 73405763 | 107867540 | −0.1761 | 0.6609 | NA | NA | NA | NA |
| WCM201_Z1_1 | X | 107869013 | 154509348 | 0.1844 | 1.1307 | NA | NA | NA | NA |
| WCM207 | 1 | 14930 | 3662308 | −0.2358 | 0.412 | 1.9145 | 0.7465 | 2 | 1 |
| WCM207 | 1 | 3662677 | 6029377 | −0.1821 | 0.4825 | 1.7441 | 1.0503 | 2 | 1 |
| WCM207 | 1 | 6038450 | 79474631 | 0.3189 | 1.1079 | 3.1785 | 1.1321 | 3 | 1 |
| WCM207 | 1 | 81967629 | 146639526 | −0.0836 | 0.61 | 1.9485 | 1.104 | 2 | 1 |
| WCM207 | 1 | 146643580 | 149872078 | −0.2276 | 0.4228 | 2.4887 | 0.1924 | 2 | 0 |
| WCM207 | 1 | 149875427 | 213349637 | −0.0212 | 0.6895 | 3.1232 | 0.1022 | 3 | 0 |
| WCM207 | 1 | 213403861 | 249214018 | −0.4797 | 0.0799 | 2.0374 | 0.0765 | 2 | 0 |
| WCM207 | 10 | 225979 | 135439049 | −0.4874 | 0.069 | 2.039 | 0.059 | 2 | 0 |
| WCM207 | 11 | 193096 | 134605959 | 0.2985 | 1.0834 | 2.119 | 2.119 | 2 | 2 |
| WCM207 | 12 | 148430 | 9849055 | −0.2947 | 0.3336 | 1.2788 | 1.2416 | 1 | 1 |
| WCM207 | 12 | 9875402 | 24737345 | −1.0977 | −0.8907 | 1.0185 | 0.0602 | 1 | 0 |
| WCM207 | 12 | 24968390 | 133810855 | −0.3519 | 0.2564 | 1.1945 | 1.1945 | 1 | 1 |
| WCM207 | 13 | 19042165 | 35739216 | 0.1898 | 0.9517 | 1.9341 | 1.9341 | 2 | 2 |
| WCM207 | 13 | 35743083 | 48667721 | −0.5361 | 0 | 1.9742 | 0.0257 | 2 | 0 |
| WCM207 | 13 | 48669223 | 49027319 | −2.4741 | NA | NA | NA | NA | NA |
| WCM207 | 13 | 49030283 | 115089411 | −0.5498 | −0.0196 | 1.9204 | 0.0526 | 2 | 0 |
| WCM207 | 14 | 19110174 | 107283263 | 0.2402 | 1.013 | 2.018 | 2.018 | 2 | 2 |
| WCM207 | 15 | 20083857 | 102506258 | −0.4698 | 0.0937 | 1.0671 | 1.0671 | 1 | 1 |
| WCM207 | 16 | 64396 | 21812102 | 0.3401 | 1.1333 | 2.1936 | 2.1936 | 2 | 2 |
| WCM207 | 16 | 21813932 | 22337593 | −0.3326 | 0.2826 | NA | NA | NA | NA |
| WCM207 | 16 | 22339794 | 29000870 | 0.3081 | 1.095 | 2.1361 | 2.1361 | 2 | 2 |
| WCM207 | 16 | 29001205 | 29974704 | −0.4355 | 0.1416 | NA | NA | NA | NA |
| WCM207 | 16 | 29974864 | 52689967 | 0.1972 | 0.9606 | 2.0174 | 1.8749 | 2 | 2 |
| WCM207 | 16 | 53190128 | 90260461 | −0.4841 | 0.0736 | 2.0298 | 0.0749 | 2 | 0 |
| WCM207 | 17 | 131595 | 18220476 | −0.5182 | 0.0255 | 2.0057 | 0.0299 | 2 | 0 |
| WCM207 | 17 | 18220859 | 40004450 | 0.2442 | 1.0179 | 2.025 | 2.025 | 2 | 2 |
| WCM207 | 17 | 40023368 | 43893780 | −0.5045 | 0.0449 | 2.023 | 0.0401 | 2 | 0 |
| WCM207 | 17 | 44039757 | 81187891 | 0.2536 | 1.0292 | 2.0409 | 2.0409 | 2 | 2 |
| WCM207 | 18 | 47779 | 13681817 | 0.2247 | 0.9942 | 1.992 | 1.992 | 2 | 2 |
| WCM207 | 18 | 13681994 | 15325957 | −0.5145 | 0.0308 | 1.8322 | 0.2109 | 2 | 0 |
| WCM207 | 18 | 18529837 | 31432784 | 0.2437 | 1.0173 | 2.0241 | 2.0241 | 2 | 2 |
| WCM207 | 18 | 31433029 | 78005189 | −0.4933 | 0.0607 | 2.0466 | 0.0394 | 2 | 0 |
| WCM207 | 19 | 105080 | 1013287 | 0.1914 | 0.9536 | 1.9603 | 1.913 | 2 | 2 |
| WCM207 | 19 | 1014398 | 3122138 | −0.529 | 0.0101 | 2.0019 | 0.0122 | 2 | 0 |
| WCM207 | 19 | 3136508 | 16977361 | 0.2471 | 1.0213 | 2.0298 | 2.0298 | 2 | 2 |
| WCM207 | 19 | 16980397 | 17438011 | −0.5049 | 0.0443 | 1.9917 | 0.0707 | 2 | 0 |
| WCM207 | 19 | 17438525 | 39669779 | 0.2165 | 0.9843 | 1.9783 | 1.9783 | 2 | 2 |
| WCM207 | 19 | 39688815 | 44174180 | −0.4723 | 0.0903 | 2.1157 | 0.0134 | 2 | 0 |
| WCM207 | 19 | 44222843 | 59093645 | 0.2348 | 1.0065 | 2.009 | 2.009 | 2 | 2 |
| WCM207 | 2 | 41602 | 71432813 | −0.4946 | 0.0589 | 1.0417 | 1.0417 | 1 | 1 |
| WCM207 | 2 | 71432918 | 73118500 | −1.0735 | −0.8477 | 1.0754 | 0.036 | 1 | 0 |
| WCM207 | 2 | 73118674 | 73497978 | −0.4655 | 0.0998 | 1.1121 | 1.0311 | 1 | 1 |
| WCM207 | 2 | 73513335 | 74690512 | −1.1123 | −0.917 | 1.0039 | 0.0553 | 1 | 0 |
| WCM207 | 2 | 74691841 | 74734906 | −0.4859 | 0.0711 | NA | NA | NA | NA |
| WCM207 | 2 | 74741940 | 85894308 | −0.2174 | 0.4363 | 1.6204 | 1.0859 | 2 | 1 |
| WCM207 | 2 | 85894946 | 86385668 | −0.9763 | −0.6797 | 1.2108 | 0.0379 | 1 | 0 |
| WCM207 | 2 | 86386755 | 243037004 | −0.1823 | 0.4823 | 1.6817 | 1.1122 | 2 | 1 |
| WCM207 | 20 | 68363 | 62934592 | 0.248 | 1.0225 | 2.0314 | 2.0314 | 2 | 2 |
| WCM207 | 21 | 9590334 | 48084202 | 0.2166 | 0.9844 | 1.9784 | 1.9784 | 2 | 2 |
| WCM207 | 22 | 16159060 | 51219138 | −0.4608 | 0.1063 | 2.0849 | 0.0681 | 2 | 0 |
| WCM207 | 3 | 239459 | 45160011 | 0.1914 | 0.9536 | 1.9367 | 1.9367 | 2 | 2 |
| WCM207 | 3 | 45266204 | 46414615 | −0.4905 | 0.0646 | 2.1206 | −0.029 | 2 | 0 |
| WCM207 | 3 | 46415115 | 47371644 | 0.2797 | 1.0608 | 2.0861 | 2.0861 | 2 | 2 |
| WCM207 | 3 | 47374842 | 49004662 | −0.4627 | 0.1037 | NA | NA | NA | NA |
| WCM207 | 3 | 49006004 | 195754093 | 0.2532 | 1.0288 | 2.0403 | 2.0403 | 2 | 2 |
| WCM207 | 3 | 195777375 | 196310987 | −0.356 | 0.2509 | 2.407 | −0.0272 | 2 | 0 |
| WCM207 | 3 | 196311203 | 197896720 | 0.2529 | 1.0284 | 2.0398 | 2.0398 | 2 | 2 |
| WCM207 | 4 | 53396 | 1135648 | −0.5112 | 0.0354 | 1.9201 | 0.1295 | 2 | 0 |
| WCM207 | 4 | 1147513 | 7461686 | 0.2119 | 0.9786 | 1.9706 | 1.9706 | 2 | 2 |
| WCM207 | 4 | 7533331 | 26491867 | −0.5132 | 0.0325 | 2.0039 | 0.0417 | 2 | 0 |
| WCM207 | 4 | 26585787 | 52961030 | 0.2479 | 1.0223 | 2.0312 | 2.0312 | 2 | 2 |
| WCM207 | 4 | 53304891 | 69484456 | −0.4956 | 0.0574 | 1.9911 | 0.09 | 2 | 0 |
| WCM207 | 4 | 69693136 | 87610220 | 0.248 | 1.0225 | 2.0314 | 2.0314 | 2 | 2 |
| WCM207 | 4 | 87614787 | 104101509 | −0.4982 | 0.0538 | 2.0661 | 0.0099 | 2 | 0 |
| WCM207 | 4 | 104101650 | 146092963 | 0.2021 | 0.9667 | 1.9543 | 1.9543 | 2 | 2 |
| WCM207 | 4 | 146095879 | 190878479 | −0.5025 | 0.0477 | 1.9987 | 0.0686 | 2 | 0 |
| WCM207 | 5 | 90751 | 32101263 | 0.1318 | 0.8805 | 1.8411 | 1.8411 | 2 | 2 |
| WCM207 | 5 | 32108141 | 32791867 | −0.5412 | −0.0074 | 1.9514 | 0.0383 | 2 | 0 |
| WCM207 | 5 | 32888214 | 180687804 | 0.2236 | 0.9928 | 1.9901 | 1.9901 | 2 | 2 |
| WCM207 | 6 | 203424 | 34883506 | 0.5163 | 1.3415 | 2.9964 | 2.072 | 3 | 2 |
| WCM207 | 6 | 34934994 | 36046518 | −0.4001 | 0.1905 | 2.3153 | −0.033 | 2 | 0 |
| WCM207 | 6 | 36063789 | 36298337 | 0.2142 | 0.9814 | NA | NA | NA | NA |
| WCM207 | 6 | 36322317 | 36652242 | −0.4438 | 0.1301 | NA | NA | NA | NA |
| WCM207 | 6 | 36653610 | 45922904 | 0.2568 | 1.0331 | 2.0464 | 2.0464 | 2 | 2 |
| WCM207 | 6 | 46028186 | 170892883 | −0.0757 | 0.6201 | 1.9341 | 1.14 | 2 | 1 |
| WCM207 | 7 | 193222 | 158937022 | 0.1721 | 0.93 | 1.9053 | 1.9053 | 2 | 2 |
| WCM207 | 8 | 163474 | 39203434 | −0.4488 | 0.1231 | 2.159 | 0.0191 | 2 | 0 |
| WCM207 | 8 | 39233365 | 39380308 | −2.2896 | NA | NA | NA | NA | NA |
| WCM207 | 8 | 39416136 | 94777889 | −0.4553 | 0.114 | 2.1156 | 0.0488 | 2 | 0 |
| WCM207 | 8 | 94781747 | 146279475 | 0.3058 | 1.0922 | 2.132 | 2.132 | 2 | 2 |
| WCM207 | 9 | 14880 | 33364781 | −0.4803 | 0.079 | 2.0705 | 0.0421 | 2 | 0 |
| WCM207 | 9 | 33366691 | 141107545 | 0.2527 | 1.0282 | 2.0395 | 2.0395 | 2 | 2 |
| WCM207 | X | 2700125 | 154493589 | 0.2443 | 1.018 | NA | NA | NA | NA |
| WCM207_X1_1 | 1 | 721476 | 1670402 | −0.1701 | 0.5123 | 1.8932 | 0.9595 | 2 | 1 |
| WCM207_X1_1 | 1 | 1684395 | 2268211 | −0.707 | −0.0292 | NA | NA | NA | NA |
| WCM207_X1_1 | 1 | 2288949 | 3350369 | −0.1469 | 0.5356 | 1.8437 | 1.0555 | 2 | 1 |
| WCM207_X1_1 | 1 | 3379975 | 3649951 | −1.6394 | −0.9752 | NA | NA | NA | NA |
| WCM207_X1_1 | 1 | 3662242 | 3784600 | −0.0834 | 0.5996 | NA | NA | NA | NA |
| WCM207_X1_1 | 1 | 3786189 | 79474645 | 0.3332 | 1.0186 | 3.0459 | 1.0059 | 3 | 1 |
| WCM207_X1_1 | 1 | 82302705 | 84880488 | −0.0437 | 0.6396 | NA | NA | NA | NA |
| WCM207_X1_1 | 1 | 84944989 | 145578433 | −0.0593 | 0.6238 | 2.0993 | 0.9826 | 2 | 1 |
| WCM207_X1_1 | 1 | 145578668 | 213405588 | −0.0525 | 0.6306 | 3.0755 | 0.0211 | 3 | 0 |
| WCM207_X1_1 | 1 | 213414282 | 249212557 | −0.5724 | 0.1067 | 2.1418 | 0.0116 | 2 | 0 |
| WCM207_X1_1 | 10 | 225988 | 135440090 | −0.6821 | −0.004 | 1.9844 | 0.01 | 2 | 0 |
| WCM207_X1_1 | 11 | 193151 | 134856542 | 0.3612 | 1.0468 | 2.0846 | 2.0472 | 2 | 2 |
| WCM207_X1_1 | 12 | 148673 | 9847457 | −0.3758 | 0.3051 | 1.517 | 0.954 | 2 | 1 |
| WCM207_X1_1 | 12 | 9871292 | 24736882 | −1.5828 | −0.9175 | 1.049 | 0.0099 | 1 | 0 |
| WCM207_X1_1 | 12 | 24970959 | 133810935 | −0.363 | 0.318 | 1.5168 | 0.9764 | 2 | 1 |
| WCM207_X1_1 | 13 | 19600159 | 35739231 | 0.2888 | 0.974 | 1.984 | 1.9445 | 2 | 2 |
| WCM207_X1_1 | 13 | 35743126 | 115089411 | −0.6807 | −0.0026 | 1.9853 | 0.0111 | 2 | 0 |
| WCM207_X1_1 | 14 | 19377733 | 107283063 | 0.2734 | 0.9585 | 1.9433 | 1.9433 | 2 | 2 |
| WCM207_X1_1 | 15 | 20170150 | 102506276 | −0.6753 | 0.0029 | 1.0789 | 0.9251 | 1 | 1 |
| WCM207_X1_1 | 16 | 67604 | 21817525 | 0.268 | 0.9531 | 1.9535 | 1.9185 | 2 | 2 |
| WCM207_X1_1 | 16 | 21847295 | 22339835 | −0.6731 | 0.005 | NA | NA | NA | NA |
| WCM207_X1_1 | 16 | 22343384 | 29001519 | 0.2725 | 0.9576 | 1.985 | 1.8991 | 2 | 2 |
| WCM207_X1_1 | 16 | 29086429 | 29974525 | −0.7341 | −0.0566 | NA | NA | NA | NA |
| WCM207_X1_1 | 16 | 29974838 | 47733249 | 0.1986 | 0.8833 | 1.9133 | 1.776 | 2 | 2 |
| WCM207_X1_1 | 16 | 48117824 | 52502463 | 0.2188 | 0.9036 | NA | NA | NA | NA |
| WCM207_X1_1 | 16 | 52553356 | 90233627 | −0.6713 | 0.0068 | 1.9891 | 0.0203 | 2 | 0 |
| WCM207_X1_1 | 17 | 131595 | 18226310 | −0.6999 | −0.022 | 1.9499 | 0.0197 | 2 | 0 |
| WCM207_X1_1 | 17 | 18232096 | 40004449 | 0.2709 | 0.956 | 1.9672 | 1.9126 | 2 | 2 |
| WCM207_X1_1 | 17 | 40024093 | 43585632 | −0.6849 | −0.0069 | 1.9683 | 0.0221 | 2 | 0 |
| WCM207_X1_1 | 17 | 43922774 | 81083455 | 0.2704 | 0.9555 | 1.947 | 1.9314 | 2 | 2 |
| WCM207_X1_1 | 18 | 48987 | 13682036 | 0.2777 | 0.9628 | 1.9922 | 1.906 | 2 | 2 |
| WCM207_X1_1 | 18 | 13731608 | 15006148 | −0.6101 | 0.0686 | NA | NA | NA | NA |
| WCM207_X1_1 | 18 | 18539817 | 31463382 | 0.2863 | 0.9715 | 2.0397 | 1.882 | 2 | 2 |
| WCM207_X1_1 | 18 | 31523089 | 78005199 | −0.6836 | −0.0056 | 1.9712 | 0.0211 | 2 | 0 |
| WCM207_X1_1 | 19 | 281506 | 1026720 | 0.2075 | 0.8923 | 1.899 | 1.8132 | 2 | 2 |
| WCM207_X1_1 | 19 | 1031109 | 3121109 | −0.7328 | −0.0553 | 1.9154 | 0.0093 | 2 | 0 |
| WCM207_X1_1 | 19 | 3136493 | 16977327 | 0.2581 | 0.9431 | 1.9305 | 1.915 | 2 | 2 |
| WCM207_X1_1 | 19 | 16980287 | 17438128 | −0.7058 | −0.028 | 1.9518 | 0.0097 | 2 | 0 |
| WCM207_X1_1 | 19 | 17438340 | 39687733 | 0.2564 | 0.9414 | 1.9416 | 1.8992 | 2 | 2 |
| WCM207_X1_1 | 19 | 39687929 | 42754695 | −0.6964 | −0.0185 | 1.9636 | 0.0108 | 2 | 0 |
| WCM207_X1_1 | 19 | 42791023 | 42796956 | 0.2443 | 0.9292 | NA | NA | NA | NA |
| WCM207_X1_1 | 19 | 42797159 | 44174243 | −0.7563 | −0.0791 | 1.8718 | 0.0215 | 2 | 0 |
| WCM207_X1_1 | 19 | 44222896 | 59083970 | 0.2375 | 0.9224 | 1.9105 | 1.88 | 2 | 2 |
| WCM207_X1_1 | 2 | 41623 | 71429659 | −0.6676 | 0.0106 | 1.0236 | 0.9911 | 1 | 1 |
| WCM207_X1_1 | 2 | 71576002 | 73118645 | −1.5991 | −0.9341 | 0.9838 | 0.063 | 1 | 0 |
| WCM207_X1_1 | 2 | 73143923 | 73635855 | −0.6715 | 0.0066 | NA | NA | NA | NA |
| WCM207_X1_1 | 2 | 73646260 | 74692039 | −1.6077 | −0.9428 | 1.0298 | 0.0106 | 1 | 0 |
| WCM207_X1_1 | 2 | 74699492 | 85875915 | −0.4137 | 0.2668 | 1.424 | 0.9823 | 1 | 1 |
| WCM207_X1_1 | 2 | 85888593 | 86378421 | −1.5773 | −0.9119 | NA | NA | NA | NA |
| WCM207_X1_1 | 2 | 86385743 | 150320685 | −0.4027 | 0.2779 | 1.446 | 0.9789 | 1 | 1 |
| WCM207_X1_1 | 2 | 150325236 | 152117867 | −1.5639 | −0.8982 | NA | NA | NA | NA |
| WCM207_X1_1 | 2 | 152127368 | 152586147 | −0.412 | 0.2685 | NA | NA | NA | NA |
| WCM207_X1_1 | 2 | 152589640 | 152830214 | −1.2938 | −0.6235 | NA | NA | NA | NA |
| WCM207_X1_1 | 2 | 152954898 | 242946616 | −0.3988 | 0.2818 | 1.4486 | 0.9828 | 1 | 1 |
| WCM207_X1_1 | 20 | 68375 | 62919134 | 0.2666 | 0.9517 | 1.9574 | 1.9108 | 2 | 2 |
| WCM207_X1_1 | 21 | 9826203 | 48084277 | 0.2634 | 0.9484 | 1.9569 | 1.9026 | 2 | 2 |
| WCM207_X1_1 | 22 | 16287434 | 51215134 | −0.6945 | −0.0166 | 1.9573 | 0.0199 | 2 | 0 |
| WCM207_X1_1 | 3 | 239350 | 45160010 | 0.2844 | 0.9695 | 1.9838 | 1.9326 | 2 | 2 |
| WCM207_X1_1 | 3 | 45187717 | 46401182 | −0.6467 | 0.0317 | NA | NA | NA | NA |
| WCM207_X1_1 | 3 | 46414687 | 47371527 | 0.3024 | 0.9876 | NA | NA | NA | NA |
| WCM207_X1_1 | 3 | 47374684 | 49004575 | −0.6836 | −0.0056 | NA | NA | NA | NA |
| WCM207_X1_1 | 3 | 49005971 | 71830739 | 0.2933 | 0.9785 | 2.0061 | 1.9348 | 2 | 2 |
| WCM207_X1_1 | 3 | 72427624 | 72881603 | −0.5772 | 0.1018 | NA | NA | NA | NA |
| WCM207_X1_1 | 3 | 72890204 | 195701321 | 0.2941 | 0.9793 | 1.9716 | 1.9716 | 2 | 2 |
| WCM207_X1_1 | 3 | 195702579 | 196311060 | −0.6326 | 0.0459 | NA | NA | NA | NA |
| WCM207_X1_1 | 3 | 196381422 | 197894712 | 0.2701 | 0.9552 | NA | NA | NA | NA |
| WCM207_X1_1 | 4 | 59339 | 1107212 | −0.6596 | 0.0186 | 2.0095 | 0.0165 | 2 | 0 |
| WCM207_X1_1 | 4 | 1161395 | 7461784 | 0.2724 | 0.9575 | 1.942 | 1.942 | 2 | 2 |
| WCM207_X1_1 | 4 | 7533287 | 26491818 | −0.7144 | −0.0367 | 1.9363 | 0.0135 | 2 | 0 |
| WCM207_X1_1 | 4 | 26585876 | 53463887 | 0.2808 | 0.966 | 1.9848 | 1.922 | 2 | 2 |
| WCM207_X1_1 | 4 | 53464785 | 62936572 | −0.7037 | −0.0259 | 1.9498 | 0.0147 | 2 | 0 |
| WCM207_X1_1 | 4 | 62937422 | 68355783 | 0.3478 | 1.0332 | NA | NA | NA | NA |
| WCM207_X1_1 | 4 | 68357940 | 69870670 | −0.7374 | −0.0599 | NA | NA | NA | NA |
| WCM207_X1_1 | 4 | 69874614 | 87610237 | 0.2836 | 0.9688 | 1.9867 | 1.9276 | 2 | 2 |
| WCM207_X1_1 | 4 | 87614754 | 104101545 | −0.6678 | 0.0104 | 1.9859 | 0.0286 | 2 | 0 |
| WCM207_X1_1 | 4 | 104102593 | 146101102 | 0.277 | 0.9621 | 1.9795 | 1.9168 | 2 | 2 |
| WCM207_X1_1 | 4 | 146435975 | 190426502 | −0.665 | 0.0132 | 2.0071 | 0.0113 | 2 | 0 |
| WCM207_X1_1 | 5 | 140579 | 32108079 | 0.0735 | 0.7575 | 1.9134 | 1.4678 | 2 | 1 |
| WCM207_X1_1 | 5 | 32126441 | 33446771 | −0.6641 | 0.0141 | NA | NA | NA | NA |
| WCM207_X1_1 | 5 | 33448684 | 180661992 | 0.2757 | 0.9609 | 1.9543 | 1.9386 | 2 | 2 |
| WCM207_X1_1 | 5 | 180662380 | 180687773 | 0.0785 | 0.7625 | NA | NA | NA | NA |
| WCM207_X1_1 | 6 | 203535 | 34855689 | 0.618 | 1.3047 | 3.0632 | 1.8775 | 3 | 2 |
| WCM207_X1_1 | 6 | 34935070 | 36063792 | −0.6523 | 0.026 | NA | NA | NA | NA |
| WCM207_X1_1 | 6 | 36068017 | 45922958 | 0.2641 | 0.9492 | 1.9483 | 1.9133 | 2 | 2 |
| WCM207_X1_1 | 6 | 46047538 | 170892614 | −0.1238 | 0.5589 | 1.9359 | 1.0104 | 2 | 1 |
| WCM207_X1_1 | 7 | 193253 | 158937135 | 0.1501 | 0.8345 | 1.9196 | 1.647 | 2 | 2 |
| WCM207_X1_1 | 8 | 190897 | 94777692 | −0.4771 | 0.2029 | 2.2888 | 0.0131 | 2 | 0 |
| WCM207_X1_1 | 8 | 94784839 | 142524822 | 0.3978 | 1.0835 | 2.1447 | 2.0936 | 2 | 2 |
| WCM207_X1_1 | 8 | 142528477 | 143447280 | −0.6667 | 0.0115 | NA | NA | NA | NA |
| WCM207_X1_1 | 8 | 143545903 | 146279416 | 0.3488 | 1.0343 | 2.2745 | 1.8217 | 2 | 2 |
| WCM207_X1_1 | 9 | 116887 | 33364754 | −0.5511 | 0.1282 | 2.174 | 0.012 | 2 | 0 |
| WCM207_X1_1 | 9 | 33366722 | 141111414 | 0.3258 | 1.0111 | 2.0277 | 2.0033 | 2 | 2 |
| WCM207_X1_1 | X | 2700146 | 154509348 | 0.2372 | 0.9221 | NA | NA | NA | NA |
| WCM212 | 1 | 14907 | 1118275 | −0.0938 | 0.5429 | 1.6532 | 1.2606 | 2 | 1 |
| WCM212 | 1 | 1119400 | 145474295 | −0.3189 | 0.1915 | 1.1419 | 1.1419 | 1 | 1 |
| WCM212 | 1 | 145474325 | 249213967 | −0.0023 | 0.6779 | 1.5998 | 1.5998 | 2 | 2 |
| WCM212 | 10 | 226116 | 105107185 | −0.3092 | 0.2073 | 2.3043 | 0.0048 | 2 | 0 |
| WCM212 | 10 | 105108455 | 105133335 | −1.5166 | −3.4838 | NA | NA | NA | NA |
| WCM212 | 10 | 105138095 | 135439016 | −0.2747 | 0.263 | 2.3775 | 0.0224 | 2 | 0 |
| WCM212 | 11 | 193096 | 2946374 | 0.1309 | 0.8675 | 2.4388 | 1.2103 | 2 | 1 |
| WCM212 | 11 | 2950322 | 20869234 | 0.0215 | 0.7123 | 1.9692 | 1.3077 | 2 | 1 |
| WCM212 | 11 | 20869336 | 102393971 | 0.115 | 0.8453 | 1.7966 | 1.7966 | 2 | 2 |
| WCM212 | 11 | 102394175 | 134090538 | 0.2252 | 0.9976 | 1.9967 | 1.9967 | 2 | 2 |
| WCM212 | 11 | 134095110 | 134605916 | 0.1703 | 0.9224 | 2.0288 | 1.7616 | 2 | 2 |
| WCM212 | 12 | 148465 | 39046712 | −0.0564 | 0.5986 | 1.5143 | 1.5143 | 2 | 2 |
| WCM212 | 12 | 39046800 | 46623349 | 0.4889 | 1.3461 | 3.47 | 1.6147 | 3 | 2 |
| WCM212 | 12 | 46633510 | 133810780 | −0.1068 | 0.5234 | 1.4374 | 1.4374 | 1 | 1 |
| WCM212 | 13 | 19041937 | 115089411 | −0.451 | −0.0312 | 1.961 | −0.0039 | 2 | 0 |
| WCM212 | 14 | 19110343 | 44976405 | 0.0987 | 0.8224 | 1.7683 | 1.7683 | 2 | 2 |
| WCM212 | 14 | 45333758 | 104245153 | 0.0043 | 0.6874 | 1.6104 | 1.6104 | 2 | 2 |
| WCM212 | 14 | 104251302 | 107283274 | 0.1704 | 0.9224 | 1.911 | 1.8796 | 2 | 2 |
| WCM212 | 15 | 20084088 | 41071399 | −0.0391 | 0.6241 | 1.5412 | 1.5412 | 2 | 2 |
| WCM212 | 15 | 41071816 | 73564881 | −0.2461 | 0.3086 | 1.2385 | 1.2385 | 1 | 1 |
| WCM212 | 15 | 73566192 | 102514188 | −0.1149 | 0.5113 | 1.4253 | 1.4253 | 1 | 1 |
| WCM212 | 16 | 64401 | 46532056 | 0.0882 | 0.8075 | 2.5667 | 0.9337 | 3 | 1 |
| WCM212 | 16 | 46534060 | 90260461 | −0.3473 | 0.1448 | 1.1055 | 1.1055 | 1 | 1 |
| WCM212 | 17 | 131595 | 21175998 | −0.2928 | 0.2339 | 2.3503 | 0.0017 | 2 | 0 |
| WCM212 | 17 | 21201719 | 81187930 | 0.1223 | 0.8555 | 1.8094 | 1.8094 | 2 | 2 |
| WCM212 | 18 | 47632 | 14124411 | −0.0559 | 0.5993 | 1.5284 | 1.5016 | 2 | 2 |
| WCM212 | 18 | 14132231 | 78005146 | −0.223 | 0.345 | 1.2702 | 1.2702 | 1 | 1 |
| WCM212 | 19 | 105021 | 1037831 | 0.3322 | 1.1416 | 2.7337 | 1.6788 | 3 | 2 |
| WCM212 | 19 | 1041340 | 24310554 | −0.0741 | 0.5724 | 2.9618 | 0.0122 | 3 | 0 |
| WCM212 | 19 | 24310774 | 40197844 | 0.64 | 1.5376 | 3.799 | 2.0071 | 4 | 2 |
| WCM212 | 19 | 40199806 | 41634029 | 1.29 | 2.3125 | 7.9045 | 2.0307 | 8 | 2 |
| WCM212 | 19 | 41697297 | 59093645 | −0.0144 | 0.6603 | 1.5804 | 1.5804 | 2 | 2 |
| WCM212 | 2 | 41686 | 39998631 | 0.0651 | 0.7749 | 1.711 | 1.711 | 2 | 2 |
| WCM212 | 2 | 40339627 | 43986123 | −0.4792 | −0.0806 | 1.9225 | −0.0312 | 2 | 0 |
| WCM212 | 2 | 43989475 | 65245765 | 0.0393 | 0.7379 | 1.6677 | 1.6677 | 2 | 2 |
| WCM212 | 2 | 65248064 | 71753481 | −0.4744 | −0.0722 | 1.9095 | −0.0071 | 2 | 0 |
| WCM212 | 2 | 71755439 | 128409127 | 0.0746 | 0.7883 | 1.7271 | 1.7271 | 2 | 2 |
| WCM212 | 2 | 128409268 | 240984637 | −0.0257 | 0.6437 | 1.5624 | 1.5624 | 2 | 2 |
| WCM212 | 2 | 240984789 | 243036990 | 0.0538 | 0.7587 | 1.7668 | 1.617 | 2 | 2 |
| WCM212 | 20 | 68427 | 10393154 | 0.4078 | 1.2411 | 2.3638 | 2.3638 | 2 | 2 |
| WCM212 | 20 | 10393469 | 60739295 | 0.2426 | 1.0214 | 2.0298 | 2.0298 | 2 | 2 |
| WCM212 | 20 | 60747704 | 62934592 | 0.3514 | 1.167 | 2.2454 | 2.2454 | 2 | 2 |
| WCM212 | 21 | 9590271 | 39947608 | 0.1048 | 0.8309 | 1.7789 | 1.7789 | 2 | 2 |
| WCM212 | 21 | 40177325 | 42866524 | −0.4685 | −0.0617 | 1.875 | 0.0413 | 2 | 0 |
| WCM212 | 21 | 42870037 | 48084210 | 0.1196 | 0.8517 | 1.8046 | 1.8046 | 2 | 2 |
| WCM212 | 22 | 16159031 | 17219250 | −0.055 | 0.6007 | NA | NA | NA | NA |
| WCM212 | 22 | 17280746 | 28503098 | 0.1104 | 0.8388 | 2.6177 | 0.9594 | 3 | 1 |
| WCM212 | 22 | 28503344 | 51223730 | −0.1184 | 0.506 | 1.8919 | 0.9484 | 2 | 1 |
| WCM212 | 3 | 239495 | 75469116 | −0.12 | 0.5037 | 1.4178 | 1.4178 | 1 | 1 |
| WCM212 | 3 | 75470091 | 197896801 | 0.2418 | 1.0202 | 2.2952 | 1.7612 | 2 | 2 |
| WCM212 | 4 | 53346 | 46042429 | −0.4236 | 0.0161 | 1.0112 | 1.0112 | 1 | 1 |
| WCM212 | 4 | 46043100 | 70261534 | −0.1549 | 0.4506 | 1.3666 | 1.3666 | 1 | 1 |
| WCM212 | 4 | 70261601 | 76957171 | 0.0826 | 0.7996 | 1.7406 | 1.7406 | 2 | 2 |
| WCM212 | 4 | 76997052 | 120528200 | −0.2342 | 0.3274 | 1.2547 | 1.2547 | 1 | 1 |
| WCM212 | 4 | 120528327 | 184243528 | −0.3522 | 0.1367 | 1.0994 | 1.0994 | 1 | 1 |
| WCM212 | 4 | 184256418 | 190878479 | −0.6848 | −0.4661 | 1.041 | 0.4068 | 1 | 0 |
| WCM212 | 5 | 90697 | 52402916 | 0.4649 | 1.3152 | 2.5013 | 2.4756 | 3 | 2 |
| WCM212 | 5 | 52404480 | 143855184 | −0.4441 | −0.0192 | 1.9449 | 0.0287 | 2 | 0 |
| WCM212 | 5 | 144577493 | 180687808 | 0.4094 | 1.2432 | 2.3673 | 2.3673 | 2 | 2 |
| WCM212 | 6 | 203516 | 11306330 | −0.5066 | −0.1293 | 1.7794 | 0.0492 | 2 | 0 |
| WCM212 | 6 | 11538569 | 27876763 | 0.0507 | 0.7543 | 1.6868 | 1.6868 | 2 | 2 |
| WCM212 | 6 | 27879080 | 62757794 | 0.207 | 0.9728 | 1.9626 | 1.9626 | 2 | 2 |
| WCM212 | 6 | 62887207 | 170892821 | 0.0322 | 0.7278 | 1.6561 | 1.6561 | 2 | 2 |
| WCM212 | 7 | 195606 | 63796744 | 0.2002 | 0.9635 | 1.974 | 1.9259 | 2 | 2 |
| WCM212 | 7 | 63797256 | 128363212 | 0.0809 | 0.7973 | 1.7378 | 1.7378 | 2 | 2 |
| WCM212 | 7 | 128364895 | 158937185 | 0.2196 | 0.99 | 2.0647 | 1.9075 | 2 | 2 |
| WCM212 | 8 | 163527 | 35402086 | −0.5623 | −0.2307 | 0.8664 | 0.838 | 1 | 1 |
| WCM212 | 8 | 35425681 | 125551423 | 0.4589 | 1.3076 | 2.4753 | 2.4753 | 2 | 2 |
| WCM212 | 8 | 125555427 | 125989339 | 2.585 | 3.7271 | NA | NA | NA | NA |
| WCM212 | 8 | 125989637 | 144351632 | 0.5144 | 1.3789 | 2.6007 | 2.6007 | 3 | 3 |
| WCM212 | 8 | 144357021 | 144407620 | 2.3154 | 3.4412 | NA | NA | NA | NA |
| WCM212 | 8 | 144411658 | 146279416 | 0.3996 | 1.2304 | 2.3463 | 2.3463 | 2 | 2 |
| WCM212 | 9 | 116873 | 12821387 | −0.1467 | 0.463 | 1.473 | 1.2838 | 1 | 1 |
| WCM212 | 9 | 12821770 | 37842209 | 0.1807 | 0.9367 | 1.9142 | 1.9142 | 2 | 2 |
| WCM212 | 9 | 37854818 | 39178322 | 0.0557 | 0.7614 | NA | NA | NA | NA |
| WCM212 | 9 | 39355656 | 132576494 | −0.1603 | 0.4422 | 1.3587 | 1.3587 | 1 | 1 |
| WCM212 | 9 | 132580791 | 141107519 | 0.0697 | 0.7813 | 1.7187 | 1.7187 | 2 | 2 |
| WCM212 | X | 2700162 | 68061897 | 0.1449 | 0.8871 | NA | NA | NA | NA |
| WCM212 | X | 68381729 | 154493484 | −0.077 | 0.568 | NA | NA | NA | NA |
| WCM212_Z2_1 | 1 | 69590 | 145115810 | −0.6242 | −0.0113 | 1.085 | 0.8995 | 1 | 1 |
| WCM212_Z2_1 | 1 | 145209427 | 249212557 | −0.0932 | 0.5634 | 1.9618 | 0.9937 | 2 | 1 |
| WCM212_Z2_1 | 10 | 225981 | 135440105 | −0.6197 | −0.0062 | 1.9808 | 0.0106 | 2 | 0 |
| WCM212_Z2_1 | 11 | 193150 | 134856556 | 0.2525 | 0.9296 | 1.9207 | 1.8887 | 2 | 2 |
| WCM212_Z2_1 | 12 | 148639 | 133810926 | −0.115 | 0.5402 | 1.962 | 0.9463 | 2 | 1 |
| WCM212_Z2_1 | 13 | 19600515 | 115089411 | −0.6215 | −0.0082 | 1.9869 | 0.0017 | 2 | 0 |
| WCM212_Z2_1 | 14 | 19377727 | 107283063 | −0.1087 | 0.5469 | 1.9554 | 0.9665 | 2 | 1 |
| WCM212_Z2_1 | 15 | 20170003 | 41556373 | −0.1363 | 0.5174 | 1.8935 | 0.9692 | 2 | 1 |
| WCM212_Z2_1 | 15 | 41562802 | 73076410 | −0.6202 | −0.0068 | 1.0194 | 0.9712 | 1 | 1 |
| WCM212_Z2_1 | 15 | 73408967 | 101427822 | −0.1384 | 0.5151 | 1.8784 | 0.9799 | 2 | 1 |
| WCM212_Z2_1 | 15 | 101432288 | 102514214 | 0.2364 | 0.9126 | 2.0726 | 1.6922 | 2 | 2 |
| WCM212_Z2_1 | 16 | 67604 | 47196555 | −0.1193 | 0.5355 | 1.9465 | 0.9525 | 2 | 1 |
| WCM212_Z2_1 | 16 | 47252840 | 90233642 | −0.6327 | −0.0205 | 1.0716 | 0.9002 | 1 | 1 |
| WCM212_Z2_1 | 17 | 131595 | 21191381 | −0.6827 | −0.0757 | 1.8908 | 0.0069 | 2 | 0 |
| WCM212_Z2_1 | 17 | 21194852 | 81083455 | 0.2063 | 0.8809 | 1.859 | 1.8241 | 2 | 2 |
| WCM212_Z2_1 | 18 | 48959 | 78005226 | −0.0845 | 0.5727 | 1.9806 | 0.994 | 2 | 1 |
| WCM212_Z2_1 | 19 | 281404 | 1041928 | 0.6196 | 1.3134 | 2.984 | 1.9865 | 3 | 2 |
| WCM212_Z2_1 | 19 | 1042104 | 24310723 | −0.0543 | 0.6049 | 3.0434 | −0.0017 | 3 | 0 |
| WCM212_Z2_1 | 19 | 29567134 | 40199954 | 1.1032 | 1.8132 | 5.1148 | 1.9136 | 5 | 2 |
| WCM212_Z2_1 | 19 | 40222033 | 41249826 | 2.1874 | 2.9187 | 13.2077 | 1.9159 | 13 | 2 |
| WCM212_Z2_1 | 19 | 41250094 | 41633847 | 1.7558 | 2.4805 | NA | NA | NA | NA |
| WCM212_Z2_1 | 19 | 41699321 | 59083929 | −0.1109 | 0.5445 | 1.9195 | 0.9975 | 2 | 1 |
| WCM212_Z2_1 | 2 | 41613 | 39998733 | 0.2967 | 0.976 | 1.967 | 1.967 | 2 | 2 |
| WCM212_Z2_1 | 2 | 40342533 | 43973047 | −0.6386 | −0.0271 | 1.9601 | 0.0026 | 2 | 0 |
| WCM212_Z2_1 | 2 | 43980780 | 44446027 | 0.3357 | 1.0169 | NA | NA | NA | NA |
| WCM212_Z2_1 | 2 | 44457576 | 65245348 | 0.2917 | 0.9707 | 1.9804 | 1.9392 | 2 | 2 |
| WCM212_Z2_1 | 2 | 65245810 | 72359573 | −0.6271 | −0.0144 | 1.9799 | 2.00E−04 | 2 | 0 |
| WCM212_Z2_1 | 2 | 72359737 | 118577231 | 0.27 | 0.9479 | 1.9635 | 1.8946 | 2 | 2 |
| WCM212_Z2_1 | 2 | 118578821 | 242946597 | −0.0666 | 0.5918 | 2.0162 | 0.998 | 2 | 1 |
| WCM212_Z2_1 | 20 | 68375 | 11904301 | 0.5784 | 1.2704 | 2.8368 | 1.988 | 3 | 2 |
| WCM212_Z2_1 | 20 | 12990016 | 43399941 | 0.2914 | 0.9704 | 1.98 | 1.9388 | 2 | 2 |
| WCM212_Z2_1 | 20 | 43530369 | 62904866 | 0.5848 | 1.2772 | 2.8928 | 1.9544 | 3 | 2 |
| WCM212_Z2_1 | 21 | 9826230 | 39822803 | 0.1749 | 0.8478 | 1.8282 | 1.7712 | 2 | 2 |
| WCM212_Z2_1 | 21 | 39947601 | 42870093 | −0.7515 | −0.1518 | 1.8292 | −0.029 | 2 | 0 |
| WCM212_Z2_1 | 21 | 43133046 | 48084210 | 0.1851 | 0.8586 | 1.8534 | 1.7731 | 2 | 2 |
| WCM212_Z2_1 | 22 | 16287470 | 28559401 | 0.2581 | 0.9355 | 2.8478 | 0.9772 | 3 | 1 |
| WCM212_Z2_1 | 22 | 28692190 | 51215153 | −0.1184 | 0.5364 | 1.9307 | 0.9701 | 2 | 1 |
| WCM212_Z2_1 | 3 | 239347 | 75476978 | −0.1314 | 0.5226 | 1.8864 | 0.9866 | 2 | 1 |
| WCM212_Z2_1 | 3 | 75478311 | 112643934 | 0.2076 | 0.8822 | 1.8801 | 1.8063 | 2 | 2 |
| WCM212_Z2_1 | 3 | 112647672 | 197894726 | 0.4944 | 1.1829 | 2.6968 | 1.8439 | 3 | 2 |
| WCM212_Z2_1 | 4 | 59331 | 17876703 | −0.6728 | −0.0648 | 1.0658 | 0.8464 | 1 | 1 |
| WCM212_Z2_1 | 4 | 17885400 | 23815522 | −1.3699 | −0.8576 | NA | NA | NA | NA |
| WCM212_Z2_1 | 4 | 23815924 | 26241363 | −0.5741 | 0.0438 | NA | NA | NA | NA |
| WCM212_Z2_1 | 4 | 26274274 | 30724790 | −1.3496 | −0.8338 | NA | NA | NA | NA |
| WCM212_Z2_1 | 4 | 30725087 | 71062426 | −0.6242 | −0.0112 | 1.0076 | 0.977 | 1 | 1 |
| WCM212_Z2_1 | 4 | 71063722 | 120987875 | −0.1261 | 0.5283 | 1.9647 | 0.9198 | 2 | 1 |
| WCM212_Z2_1 | 4 | 121616352 | 124235105 | −0.6188 | −0.0052 | NA | NA | NA | NA |
| WCM212_Z2_1 | 4 | 124323012 | 128584580 | −0.1213 | 0.5334 | NA | NA | NA | NA |
| WCM212_Z2_1 | 4 | 128590245 | 184368308 | −0.5825 | 0.0346 | 1.0501 | 0.9984 | 1 | 1 |
| WCM212_Z2_1 | 4 | 184368582 | 185350159 | −1.3567 | −0.8421 | NA | NA | NA | NA |
| WCM212_Z2_1 | 4 | 185550524 | 186097061 | −0.4836 | 0.1427 | NA | NA | NA | NA |
| WCM212_Z2_1 | 4 | 186111299 | 190426570 | −1.4686 | −0.9748 | 1.0388 | −0.0211 | 1 | 0 |
| WCM212_Z2_1 | 5 | 140666 | 52405544 | 0.2868 | 0.9656 | 1.9959 | 1.9098 | 2 | 2 |
| WCM212_Z2_1 | 5 | 52778714 | 143853636 | −0.6146 | −7.00E−04 | 1.9859 | 0.0131 | 2 | 0 |
| WCM212_Z2_1 | 5 | 145144521 | 180687776 | 0.2819 | 0.9605 | 1.9562 | 1.9357 | 2 | 2 |
| WCM212_Z2_1 | 6 | 203475 | 11565979 | −0.6182 | −0.0046 | 1.9895 | 0.0041 | 2 | 0 |
| WCM212_Z2_1 | 6 | 11575661 | 170893415 | 0.2176 | 0.8928 | 1.8685 | 1.845 | 2 | 2 |
| WCM212_Z2_1 | 7 | 193222 | 158937400 | 0.3149 | 0.9951 | 2.0183 | 1.9681 | 2 | 2 |
| WCM212_Z2_1 | 8 | 190908 | 35650786 | −0.6523 | −0.0421 | 0.9873 | 0.9552 | 1 | 1 |
| WCM212_Z2_1 | 8 | 36641976 | 146279507 | 0.2723 | 0.9503 | 2.8747 | 0.9898 | 3 | 1 |
| WCM212_Z2_1 | 9 | 116902 | 69256890 | 0.2676 | 0.9454 | 1.952 | 1.8994 | 2 | 2 |
| WCM212_Z2_1 | 9 | 71144493 | 132576451 | −0.1083 | 0.5473 | 1.9309 | 0.9917 | 2 | 1 |
| WCM212_Z2_1 | 9 | 132581031 | 141111414 | 0.2607 | 0.9382 | 1.9161 | 1.9161 | 2 | 2 |
| WCM212_Z2_1 | X | 2700145 | 154509348 | 0.2483 | 0.9251 | NA | NA | NA | NA |
| WCM213 | 1 | 14930 | 23609333 | 0.0663 | 0.2994 | 1.2306 | 1.2306 | 1 | 1 |
| WCM213 | 1 | 23636234 | 156737969 | −0.0097 | −0.155 | 0.8981 | 0.8981 | 1 | 1 |
| WCM213 | 1 | 156752048 | 249213967 | 0.0872 | 0.4063 | 1.3253 | 1.3253 | 1 | 1 |
| WCM213 | 10 | 225958 | 135438959 | −0.0092 | −0.1512 | 0.9005 | 0.9005 | 1 | 1 |
| WCM213 | 11 | 193112 | 62334787 | 0.0062 | −0.0496 | 0.9662 | 0.9662 | 1 | 1 |
| WCM213 | 11 | 62335042 | 65835438 | −0.23 | −5.1812 | 0.0276 | 0.0276 | 0 | 0 |
| WCM213 | 11 | 65836198 | 134605959 | −0.01 | −0.1572 | 0.8967 | 0.8967 | 1 | 1 |
| WCM213 | 12 | 148576 | 133810869 | −0.0231 | −0.2494 | 0.8412 | 0.8412 | 1 | 1 |
| WCM213 | 13 | 19041994 | 115089411 | −0.0566 | −0.5118 | 0.7014 | 0.7014 | 1 | 1 |
| WCM213 | 14 | 19110251 | 107283270 | −0.006 | −0.1298 | 0.9139 | 0.9139 | 1 | 1 |
| WCM213 | 15 | 20084061 | 102514249 | 0.0099 | −0.026 | 0.9821 | 0.9821 | 1 | 1 |
| WCM213 | 16 | 67256 | 90168697 | 0.0127 | −0.008 | 0.9945 | 0.9945 | 1 | 1 |
| WCM213 | 17 | 262900 | 81187853 | −0.0131 | −0.1784 | 0.8837 | 0.8837 | 1 | 1 |
| WCM213 | 18 | 47806 | 78005203 | −0.0209 | −0.2334 | 0.8506 | 0.8506 | 1 | 1 |
| WCM213 | 19 | 105050 | 59093645 | −0.0374 | −0.3562 | 0.7812 | 0.7812 | 1 | 1 |
| WCM213 | 2 | 41612 | 243052762 | −0.0176 | −0.2097 | 0.8647 | 0.8647 | 1 | 1 |
| WCM213 | 20 | 68340 | 62934592 | 0.0378 | 0.1429 | 1.1041 | 1.1041 | 1 | 1 |
| WCM213 | 21 | 9590301 | 48084196 | 0.036 | 0.1325 | 1.0962 | 1.0962 | 1 | 1 |
| WCM213 | 22 | 16159060 | 51219090 | 0.023 | 0.0555 | 1.0392 | 1.0392 | 1 | 1 |
| WCM213 | 3 | 239524 | 121591830 | −0.0048 | −0.1215 | 0.9193 | 0.9193 | 1 | 1 |
| WCM213 | 3 | 121605171 | 140397288 | 0.1466 | 0.6795 | 1.6015 | 1.6015 | 2 | 2 |
| WCM213 | 3 | 140401351 | 197896818 | −0.0346 | −0.3343 | 0.7932 | 0.7932 | 1 | 1 |
| WCM213 | 4 | 53377 | 190903980 | 6.00E−04 | −0.0859 | 0.9422 | 0.9422 | 1 | 1 |
| WCM213 | 5 | 90647 | 55238586 | 0.051 | 0.2168 | 1.1622 | 1.1622 | 1 | 1 |
| WCM213 | 5 | 55240490 | 145849065 | −0.0528 | −0.4796 | 0.7172 | 0.7172 | 1 | 1 |
| WCM213 | 5 | 145849345 | 180687854 | 0.0524 | 0.2249 | 1.1687 | 1.1687 | 1 | 1 |
| WCM213 | 6 | 203479 | 170892848 | −0.0249 | −0.2627 | 0.8336 | 0.8336 | 1 | 1 |
| WCM213 | 7 | 193222 | 158937072 | 0.0611 | 0.2717 | 1.2072 | 1.2072 | 1 | 1 |
| WCM213 | 8 | 163527 | 65489024 | −0.0204 | −0.2299 | 0.8527 | 0.8527 | 1 | 1 |
| WCM213 | 8 | 65489135 | 145666371 | 0.1511 | 0.6986 | 1.623 | 1.623 | 2 | 2 |
| WCM213 | 8 | 145667516 | 146279480 | −0.0107 | −0.162 | 0.9921 | 0.7955 | 1 | 1 |
| WCM213 | 9 | 116800 | 141107533 | −9.00E−04 | −0.0955 | 0.936 | 0.936 | 1 | 1 |
| WCM213 | X | 2700202 | 154493478 | −0.0371 | −0.354 | NA | NA | NA | NA |
| WCM213_X1_1 | 1 | 721415 | 880916 | 0.317 | NA | NA | NA | NA | NA |
| WCM213_X1_1 | 1 | 881627 | 10463163 | −0.0117 | NA | NA | NA | NA | NA |
| WCM213_X1_1 | 1 | 10464227 | 70883666 | −0.149 | NA | NA | NA | NA | NA |
| WCM213_X1_1 | 1 | 70887343 | 102290624 | 0.004 | NA | NA | NA | NA | NA |
| WCM213_X1_1 | 1 | 102296279 | 120611454 | −0.3643 | NA | NA | NA | NA | NA |
| WCM213_X1_1 | 1 | 120611948 | 150913872 | 0.7462 | NA | NA | NA | NA | NA |
| WCM213_X1_1 | 1 | 150915083 | 242042621 | 0.4759 | NA | NA | NA | NA | NA |
| WCM213_X1_1 | 1 | 242045259 | 249212373 | 0.6624 | NA | NA | NA | NA | NA |
| WCM213_X1_1 | 10 | 255940 | 81700492 | −0.0843 | NA | NA | NA | NA | NA |
| WCM213_X1_1 | 10 | 81701219 | 105203787 | −0.5466 | NA | NA | NA | NA | NA |
| WCM213_X1_1 | 10 | 105204367 | 135440117 | −0.2389 | NA | NA | NA | NA | NA |
| WCM213_X1_1 | 11 | 193112 | 59516849 | −0.1067 | NA | NA | NA | NA | NA |
| WCM213_X1_1 | 11 | 59523088 | 65888527 | −0.3477 | NA | NA | NA | NA | NA |
| WCM213_X1_1 | 11 | 65960986 | 68177550 | 0.166 | NA | NA | NA | NA | NA |
| WCM213_X1_1 | 11 | 68178928 | 134180978 | −0.117 | NA | NA | NA | NA | NA |
| WCM213_X1_1 | 11 | 134182254 | 134856559 | 0.0839 | NA | NA | NA | NA | NA |
| WCM213_X1_1 | 12 | 148642 | 110295384 | −0.2751 | NA | NA | NA | NA | NA |
| WCM213_X1_1 | 12 | 110295991 | 133318071 | −0.0202 | NA | NA | NA | NA | NA |
| WCM213_X1_1 | 12 | 133318406 | 133810868 | −0.2612 | NA | NA | NA | NA | NA |
| WCM213_X1_1 | 13 | 19600515 | 115089411 | −0.1374 | NA | NA | NA | NA | NA |
| WCM213_X1_1 | 14 | 19377698 | 73581029 | −0.0709 | NA | NA | NA | NA | NA |
| WCM213_X1_1 | 14 | 73614814 | 75142480 | 0.3656 | NA | NA | NA | NA | NA |
| WCM213_X1_1 | 14 | 75142987 | 75264565 | 0.1564 | NA | NA | NA | NA | NA |
| WCM213_X1_1 | 14 | 75264735 | 103355925 | −0.1102 | NA | NA | NA | NA | NA |
| WCM213_X1_1 | 14 | 103357737 | 107283087 | 0.1409 | NA | NA | NA | NA | NA |
| WCM213_X1_1 | 15 | 20170134 | 41030170 | −0.1495 | NA | NA | NA | NA | NA |
| WCM213_X1_1 | 15 | 41030715 | 60656628 | −0.0244 | NA | NA | NA | NA | NA |
| WCM213_X1_1 | 15 | 60666686 | 102517678 | −0.1112 | NA | NA | NA | NA | NA |
| WCM213_X1_1 | 16 | 67604 | 2164652 | −0.0975 | NA | NA | NA | NA | NA |
| WCM213_X1_1 | 16 | 2166628 | 2321748 | 0.0867 | NA | NA | NA | NA | NA |
| WCM213_X1_1 | 16 | 2326679 | 21156619 | 0.1496 | NA | NA | NA | NA | NA |
| WCM213_X1_1 | 16 | 21157383 | 87448043 | 0.0357 | NA | NA | NA | NA | NA |
| WCM213_X1_1 | 16 | 87448935 | 90162182 | −0.0381 | NA | NA | NA | NA | NA |
| WCM213_X1_1 | 17 | 131595 | 7230144 | 0.0217 | NA | NA | NA | NA | NA |
| WCM213_X1_1 | 17 | 7230486 | 15848844 | −0.1972 | NA | NA | NA | NA | NA |
| WCM213_X1_1 | 17 | 15878276 | 16734839 | −0.7701 | NA | NA | NA | NA | NA |
| WCM213_X1_1 | 17 | 16735643 | 19607457 | −0.2538 | NA | NA | NA | NA | NA |
| WCM213_X1_1 | 17 | 19607989 | 40629672 | 0.0992 | NA | NA | NA | NA | NA |
| WCM213_X1_1 | 17 | 40630510 | 42295575 | −0.2195 | NA | NA | NA | NA | NA |
| WCM213_X1_1 | 17 | 42327850 | 44954069 | 0.1555 | NA | NA | NA | NA | NA |
| WCM213_X1_1 | 17 | 44962398 | 64800116 | 0.3046 | NA | NA | NA | NA | NA |
| WCM213_X1_1 | 17 | 64873552 | 81083526 | 0.1014 | NA | NA | NA | NA | NA |
| WCM213_X1_1 | 18 | 48202 | 33703547 | 0.1314 | NA | NA | NA | NA | NA |
| WCM213_X1_1 | 18 | 33704532 | 78005215 | −0.1502 | NA | NA | NA | NA | NA |
| WCM213_X1_1 | 19 | 281405 | 8842313 | −0.2977 | NA | NA | NA | NA | NA |
| WCM213_X1_1 | 19 | 8931884 | 43441188 | −0.0778 | NA | NA | NA | NA | NA |
| WCM213_X1_1 | 19 | 43514159 | 43708392 | −0.145 | NA | NA | NA | NA | NA |
| WCM213_X1_1 | 19 | 43709006 | 59083968 | 0.2312 | NA | NA | NA | NA | NA |
| WCM213_X1_1 | 2 | 41612 | 33526713 | −0.0085 | NA | NA | NA | NA | NA |
| WCM213_X1_1 | 2 | 33534529 | 75425728 | −0.2991 | NA | NA | NA | NA | NA |
| WCM213_X1_1 | 2 | 75425942 | 84869148 | −0.1128 | NA | NA | NA | NA | NA |
| WCM213_X1_1 | 2 | 84880445 | 209358277 | 0.0905 | NA | NA | NA | NA | NA |
| WCM213_X1_1 | 2 | 209436747 | 242946616 | −0.2041 | NA | NA | NA | NA | NA |
| WCM213_X1_1 | 20 | 68363 | 49219097 | 0.144 | NA | NA | NA | NA | NA |
| WCM213_X1_1 | 20 | 49219132 | 62863548 | 0.5424 | NA | NA | NA | NA | NA |
| WCM213_X1_1 | 20 | 62867958 | 62904847 | 0.0143 | NA | NA | NA | NA | NA |
| WCM213_X1_1 | 21 | 9826225 | 40177454 | 0.1639 | NA | NA | NA | NA | NA |
| WCM213_X1_1 | 21 | 40177902 | 43164295 | −0.4651 | NA | NA | NA | NA | NA |
| WCM213_X1_1 | 21 | 43165986 | 48084282 | 0.3015 | NA | NA | NA | NA | NA |
| WCM213_X1_1 | 22 | 16287446 | 51215142 | −0.0118 | NA | NA | NA | NA | NA |
| WCM213_X1_1 | 3 | 239431 | 93772078 | −0.0504 | NA | NA | NA | NA | NA |
| WCM213_X1_1 | 3 | 93779860 | 107647064 | 0.3842 | NA | NA | NA | NA | NA |
| WCM213_X1_1 | 3 | 107766138 | 121438530 | −0.0074 | NA | NA | NA | NA | NA |
| WCM213_X1_1 | 3 | 121441126 | 140981565 | 0.342 | NA | NA | NA | NA | NA |
| WCM213_X1_1 | 3 | 140997285 | 143704528 | 0.0562 | NA | NA | NA | NA | NA |
| WCM213_X1_1 | 3 | 143708450 | 180426604 | −0.4314 | NA | NA | NA | NA | NA |
| WCM213_X1_1 | 3 | 180466038 | 197894719 | 0.0079 | NA | NA | NA | NA | NA |
| WCM213_X1_1 | 4 | 59382 | 155394 | −0.3894 | NA | NA | NA | NA | NA |
| WCM213_X1_1 | 4 | 155713 | 7026855 | 0.059 | NA | NA | NA | NA | NA |
| WCM213_X1_1 | 4 | 7026987 | 23803456 | 0.2145 | NA | NA | NA | NA | NA |
| WCM213_X1_1 | 4 | 23803869 | 71690040 | −0.0718 | NA | NA | NA | NA | NA |
| WCM213_X1_1 | 4 | 71691144 | 89597367 | 0.2138 | NA | NA | NA | NA | NA |
| WCM213_X1_1 | 4 | 89599185 | 146791544 | 0.0094 | NA | NA | NA | NA | NA |
| WCM213_X1_1 | 4 | 146806947 | 190426502 | −0.4002 | NA | NA | NA | NA | NA |
| WCM213_X1_1 | 5 | 140532 | 59064184 | −0.0014 | NA | NA | NA | NA | NA |
| WCM213_X1_1 | 5 | 59189111 | 96071925 | −0.3475 | NA | NA | NA | NA | NA |
| WCM213_X1_1 | 5 | 96073629 | 131411460 | −0.0919 | NA | NA | NA | NA | NA |
| WCM213_X1_1 | 5 | 131530678 | 134130657 | −0.3565 | NA | NA | NA | NA | NA |
| WCM213_X1_1 | 5 | 134131812 | 180687814 | −0.0089 | NA | NA | NA | NA | NA |
| WCM213_X1_1 | 6 | 203542 | 10530042 | −0.2536 | NA | NA | NA | NA | NA |
| WCM213_X1_1 | 6 | 10556811 | 57055356 | −0.0324 | NA | NA | NA | NA | NA |
| WCM213_X1_1 | 6 | 57059617 | 74446196 | −0.1469 | NA | NA | NA | NA | NA |
| WCM213_X1_1 | 6 | 74468745 | 118024816 | −0.3926 | NA | NA | NA | NA | NA |
| WCM213_X1_1 | 6 | 118475611 | 170893424 | −0.2158 | NA | NA | NA | NA | NA |
| WCM213_X1_1 | 7 | 193253 | 37243784 | −0.3196 | NA | NA | NA | NA | NA |
| WCM213_X1_1 | 7 | 37251018 | 99375664 | 0.2418 | NA | NA | NA | NA | NA |
| WCM213_X1_1 | 7 | 99377695 | 110762898 | 0.535 | NA | NA | NA | NA | NA |
| WCM213_X1_1 | 7 | 110763304 | 158937400 | 0.2897 | NA | NA | NA | NA | NA |
| WCM213_X1_1 | 8 | 190906 | 1853827 | −0.7279 | NA | NA | NA | NA | NA |
| WCM213_X1_1 | 8 | 1857548 | 16921612 | −0.3875 | NA | NA | NA | NA | NA |
| WCM213_X1_1 | 8 | 16935331 | 22990099 | −0.6869 | NA | NA | NA | NA | NA |
| WCM213_X1_1 | 8 | 22995427 | 25267622 | −0.059 | NA | NA | NA | NA | NA |
| WCM213_X1_1 | 8 | 25268743 | 37374035 | −0.4381 | NA | NA | NA | NA | NA |
| WCM213_X1_1 | 8 | 37374363 | 63930163 | 0.1923 | NA | NA | NA | NA | NA |
| WCM213_X1_1 | 8 | 63936699 | 79629612 | 0.4859 | NA | NA | NA | NA | NA |
| WCM213_X1_1 | 8 | 79648711 | 89198712 | 0.9379 | NA | NA | NA | NA | NA |
| WCM213_X1_1 | 8 | 89209504 | 131848566 | 0.4695 | NA | NA | NA | NA | NA |
| WCM213_X1_1 | 8 | 131859743 | 146279480 | 0.2895 | NA | NA | NA | NA | NA |
| WCM213_X1_1 | 9 | 17079 | 35706249 | −0.1987 | NA | NA | NA | NA | NA |
| WCM213_X1_1 | 9 | 35706488 | 111903799 | −0.3679 | NA | NA | NA | NA | NA |
| WCM213_X1_1 | 9 | 111909417 | 141111414 | −0.2855 | NA | NA | NA | NA | NA |
| WCM213_X1_1 | X | 2700159 | 67431965 | 0.6698 | NA | NA | NA | NA | NA |
| WCM213_X1_1 | X | 67454333 | 69176946 | −0.2318 | NA | NA | NA | NA | NA |
| WCM213_X1_1 | X | 69242765 | 74960745 | 0.1355 | NA | NA | NA | NA | NA |
| WCM213_X1_1 | X | 74961034 | 154509348 | −0.2841 | NA | NA | NA | NA | NA |
| WCM213_Z13_1 | 1 | 721415 | 49208347 | −0.0713 | NA | NA | NA | NA | NA |
| WCM213_Z13_1 | 1 | 49224803 | 70899534 | −0.176 | NA | NA | NA | NA | NA |
| WCM213_Z13_1 | 1 | 70900851 | 79402067 | 0.1411 | NA | NA | NA | NA | NA |
| WCM213_Z13_1 | 1 | 79403501 | 93826157 | −0.1906 | NA | NA | NA | NA | NA |
| WCM213_Z13_1 | 1 | 93965033 | 102290649 | 0.1582 | NA | NA | NA | NA | NA |
| WCM213_Z13_1 | 1 | 102296279 | 120612014 | −0.4902 | NA | NA | NA | NA | NA |
| WCM213_Z13_1 | 1 | 143767833 | 153751862 | 0.3 | NA | NA | NA | NA | NA |
| WCM213_Z13_1 | 1 | 153752478 | 155582876 | 0.6297 | NA | NA | NA | NA | NA |
| WCM213_Z13_1 | 1 | 155583287 | 155982353 | 0.262 | NA | NA | NA | NA | NA |
| WCM213_Z13_1 | 1 | 155984760 | 179873099 | 0.1275 | NA | NA | NA | NA | NA |
| WCM213_Z13_1 | 1 | 179877780 | 225609897 | 0.2621 | NA | NA | NA | NA | NA |
| WCM213_Z13_1 | 1 | 225611661 | 226497271 | −0.1495 | NA | NA | NA | NA | NA |
| WCM213_Z13_1 | 1 | 226497483 | 249212429 | 0.3001 | NA | NA | NA | NA | NA |
| WCM213_Z13_1 | 10 | 255871 | 81697868 | −0.0208 | NA | NA | NA | NA | NA |
| WCM213_Z13_1 | 10 | 81700493 | 105108750 | −0.3724 | NA | NA | NA | NA | NA |
| WCM213_Z13_1 | 10 | 105110649 | 135440105 | −0.0791 | NA | NA | NA | NA | NA |
| WCM213_Z13_1 | 11 | 193112 | 34378465 | −0.1458 | NA | NA | NA | NA | NA |
| WCM213_Z13_1 | 11 | 34378580 | 59480980 | −0.0049 | NA | NA | NA | NA | NA |
| WCM213_Z13_1 | 11 | 59481155 | 65961018 | −0.2449 | NA | NA | NA | NA | NA |
| WCM213_Z13_1 | 11 | 65977871 | 69978074 | 0.0858 | NA | NA | NA | NA | NA |
| WCM213_Z13_1 | 11 | 69995893 | 76076986 | −0.1394 | NA | NA | NA | NA | NA |
| WCM213_Z13_1 | 11 | 76091814 | 83874546 | 0.1448 | NA | NA | NA | NA | NA |
| WCM213_Z13_1 | 11 | 83877999 | 134856559 | −0.1625 | NA | NA | NA | NA | NA |
| WCM213_Z13_1 | 12 | 148644 | 416249 | 0.0655 | NA | NA | NA | NA | NA |
| WCM213_Z13_1 | 12 | 416977 | 14664562 | −0.0417 | NA | NA | NA | NA | NA |
| WCM213_Z13_1 | 12 | 14688651 | 68618970 | −0.1765 | NA | NA | NA | NA | NA |
| WCM213_Z13_1 | 12 | 68619233 | 110246136 | −0.0511 | NA | NA | NA | NA | NA |
| WCM213_Z13_1 | 12 | 110252458 | 111872722 | 0.1733 | NA | NA | NA | NA | NA |
| WCM213_Z13_1 | 12 | 111884608 | 113496222 | 0.4112 | NA | NA | NA | NA | NA |
| WCM213_Z13_1 | 12 | 113515269 | 133810842 | 0.1179 | NA | NA | NA | NA | NA |
| WCM213_Z13_1 | 13 | 19600515 | 115089411 | −0.2213 | NA | NA | NA | NA | NA |
| WCM213_Z13_1 | 14 | 19377723 | 47687273 | 0.0035 | NA | NA | NA | NA | NA |
| WCM213_Z13_1 | 14 | 47770744 | 53619818 | 0.4714 | NA | NA | NA | NA | NA |
| WCM213_Z13_1 | 14 | 54416948 | 73576161 | −0.0508 | NA | NA | NA | NA | NA |
| WCM213_Z13_1 | 14 | 73577565 | 76525685 | 0.2183 | NA | NA | NA | NA | NA |
| WCM213_Z13_1 | 14 | 76543004 | 107283220 | −0.0311 | NA | NA | NA | NA | NA |
| WCM213_Z13_1 | 15 | 20170150 | 31515782 | −0.209 | NA | NA | NA | NA | NA |
| WCM213_Z13_1 | 15 | 31521493 | 102358660 | −0.0874 | NA | NA | NA | NA | NA |
| WCM213_Z13_1 | 15 | 102359097 | 102517678 | −0.0867 | NA | NA | NA | NA | NA |
| WCM213_Z13_1 | 16 | 67604 | 2354058 | 0.0066 | NA | NA | NA | NA | NA |
| WCM213_Z13_1 | 16 | 2358567 | 3658573 | 0.5421 | NA | NA | NA | NA | NA |
| WCM213_Z13_1 | 16 | 3658716 | 16138322 | 0.6321 | NA | NA | NA | NA | NA |
| WCM213_Z13_1 | 16 | 16139714 | 27629752 | 0.5625 | NA | NA | NA | NA | NA |
| WCM213_Z13_1 | 16 | 27640103 | 90162178 | 0.0091 | NA | NA | NA | NA | NA |
| WCM213_Z13_1 | 17 | 131595 | 6903715 | −0.0229 | NA | NA | NA | NA | NA |
| WCM213_Z13_1 | 17 | 6905061 | 15848882 | −0.2137 | NA | NA | NA | NA | NA |
| WCM213_Z13_1 | 17 | 15878256 | 16734866 | −0.822 | NA | NA | NA | NA | NA |
| WCM213_Z13_1 | 17 | 16735640 | 19605934 | −0.2425 | NA | NA | NA | NA | NA |
| WCM213_Z13_1 | 17 | 19606468 | 22023461 | 0.105 | NA | NA | NA | NA | NA |
| WCM213_Z13_1 | 17 | 25621436 | 27186031 | 0.4366 | NA | NA | NA | NA | NA |
| WCM213_Z13_1 | 17 | 27186274 | 34964703 | 0.0754 | NA | NA | NA | NA | NA |
| WCM213_Z13_1 | 17 | 35294486 | 37119226 | −0.3237 | NA | NA | NA | NA | NA |
| WCM213_Z13_1 | 17 | 37219748 | 40575101 | 0.0813 | NA | NA | NA | NA | NA |
| WCM213_Z13_1 | 17 | 40612954 | 42293927 | −0.3477 | NA | NA | NA | NA | NA |
| WCM213_Z13_1 | 17 | 42295568 | 67075142 | 0.0974 | NA | NA | NA | NA | NA |
| WCM213_Z13_1 | 17 | 67075396 | 70117575 | −0.3819 | NA | NA | NA | NA | NA |
| WCM213_Z13_1 | 17 | 70117819 | 81083528 | 0.1004 | NA | NA | NA | NA | NA |
| WCM213_Z13_1 | 18 | 48259 | 18603627 | 0.1015 | NA | NA | NA | NA | NA |
| WCM213_Z13_1 | 18 | 18608748 | 29435667 | −0.0996 | NA | NA | NA | NA | NA |
| WCM213_Z13_1 | 18 | 29437760 | 39537593 | 0.1683 | NA | NA | NA | NA | NA |
| WCM213_Z13_1 | 18 | 39542526 | 78005210 | −0.3755 | NA | NA | NA | NA | NA |
| WCM213_Z13_1 | 19 | 281468 | 8842274 | −0.3704 | NA | NA | NA | NA | NA |
| WCM213_Z13_1 | 19 | 8931865 | 14772866 | −0.2344 | NA | NA | NA | NA | NA |
| WCM213_Z13_1 | 19 | 14774238 | 14828507 | −0.4725 | NA | NA | NA | NA | NA |
| WCM213_Z13_1 | 19 | 14829105 | 46319815 | −0.0978 | NA | NA | NA | NA | NA |
| WCM213_Z13_1 | 19 | 46320066 | 59083953 | 0.0411 | NA | NA | NA | NA | NA |
| WCM213_Z13_1 | 2 | 41623 | 32148721 | 0.0243 | NA | NA | NA | NA | NA |
| WCM213_Z13_1 | 2 | 32157206 | 68717345 | −0.3045 | NA | NA | NA | NA | NA |
| WCM213_Z13_1 | 2 | 68729916 | 172930997 | −0.0907 | NA | NA | NA | NA | NA |
| WCM213_Z13_1 | 2 | 172935758 | 172967005 | −0.2211 | NA | NA | NA | NA | NA |
| WCM213_Z13_1 | 2 | 172967171 | 209358295 | 0.0584 | NA | NA | NA | NA | NA |
| WCM213_Z13_1 | 2 | 209436747 | 242946597 | −0.2669 | NA | NA | NA | NA | NA |
| WCM213_Z13_1 | 20 | 68406 | 11903839 | −0.2426 | NA | NA | NA | NA | NA |
| WCM213_Z13_1 | 20 | 11904105 | 45700876 | −0.0696 | NA | NA | NA | NA | NA |
| WCM213_Z13_1 | 20 | 45702935 | 48892255 | −0.2563 | NA | NA | NA | NA | NA |
| WCM213_Z13_1 | 20 | 48894093 | 62868734 | 0.4315 | NA | NA | NA | NA | NA |
| WCM213_Z13_1 | 20 | 62871232 | 62904863 | 0.1003 | NA | NA | NA | NA | NA |
| WCM213_Z13_1 | 21 | 9826225 | 39822803 | 0.3015 | NA | NA | NA | NA | NA |
| WCM213_Z13_1 | 21 | 39947608 | 43133055 | −0.5405 | NA | NA | NA | NA | NA |
| WCM213_Z13_1 | 21 | 43133829 | 48084226 | 0.2816 | NA | NA | NA | NA | NA |
| WCM213_Z13_1 | 22 | 16287470 | 51215134 | 0.0202 | NA | NA | NA | NA | NA |
| WCM213_Z13_1 | 3 | 239398 | 121500699 | 0.0177 | NA | NA | NA | NA | NA |
| WCM213_Z13_1 | 3 | 121507186 | 132400812 | 0.4483 | NA | NA | NA | NA | NA |
| WCM213_Z13_1 | 3 | 132401599 | 137893494 | 0.2004 | NA | NA | NA | NA | NA |
| WCM213_Z13_1 | 3 | 137907373 | 138034421 | 0.4203 | NA | NA | NA | NA | NA |
| WCM213_Z13_1 | 3 | 138037051 | 145782435 | 0.103 | NA | NA | NA | NA | NA |
| WCM213_Z13_1 | 3 | 145788513 | 157081393 | −0.4181 | NA | NA | NA | NA | NA |
| WCM213_Z13_1 | 3 | 157082200 | 197894713 | −0.0618 | NA | NA | NA | NA | NA |
| WCM213_Z13_1 | 4 | 59390 | 7064095 | −0.1629 | NA | NA | NA | NA | NA |
| WCM213_Z13_1 | 4 | 7065881 | 103806495 | 0.086 | NA | NA | NA | NA | NA |
| WCM213_Z13_1 | 4 | 103832690 | 104640818 | 0.5384 | NA | NA | NA | NA | NA |
| WCM213_Z13_1 | 4 | 105393505 | 126412765 | 0.0765 | NA | NA | NA | NA | NA |
| WCM213_Z13_1 | 4 | 128544612 | 151114357 | 0.2821 | NA | NA | NA | NA | NA |
| WCM213_Z13_1 | 4 | 151119170 | 190426471 | −0.3147 | NA | NA | NA | NA | NA |
| WCM213_Z13_1 | 5 | 140532 | 56255217 | 0.1721 | NA | NA | NA | NA | NA |
| WCM213_Z13_1 | 5 | 56509999 | 93489726 | −0.2485 | NA | NA | NA | NA | NA |
| WCM213_Z13_1 | 5 | 93720096 | 118486102 | −0.0995 | NA | NA | NA | NA | NA |
| WCM213_Z13_1 | 5 | 118487676 | 131410568 | 0.1616 | NA | NA | NA | NA | NA |
| WCM213_Z13_1 | 5 | 131411460 | 131553496 | −0.1457 | NA | NA | NA | NA | NA |
| WCM213_Z13_1 | 5 | 131554241 | 134147383 | −0.2427 | NA | NA | NA | NA | NA |
| WCM213_Z13_1 | 5 | 134152195 | 180687808 | 0.2084 | NA | NA | NA | NA | NA |
| WCM213_Z13_1 | 6 | 203515 | 10529797 | −0.2725 | NA | NA | NA | NA | NA |
| WCM213_Z13_1 | 6 | 10529956 | 10830846 | −0.1236 | NA | NA | NA | NA | NA |
| WCM213_Z13_1 | 6 | 10874272 | 18406327 | 0.1965 | NA | NA | NA | NA | NA |
| WCM213_Z13_1 | 6 | 18427820 | 28963248 | −0.197 | NA | NA | NA | NA | NA |
| WCM213_Z13_1 | 6 | 28963364 | 36564739 | 0.1187 | NA | NA | NA | NA | NA |
| WCM213_Z13_1 | 6 | 36566629 | 41900435 | −0.1213 | NA | NA | NA | NA | NA |
| WCM213_Z13_1 | 6 | 41903783 | 49580247 | 0.1181 | NA | NA | NA | NA | NA |
| WCM213_Z13_1 | 6 | 49582512 | 74520803 | −0.0949 | NA | NA | NA | NA | NA |
| WCM213_Z13_1 | 6 | 74521964 | 80201400 | −0.28 | NA | NA | NA | NA | NA |
| WCM213_Z13_1 | 6 | 80202326 | 99374815 | −0.2542 | NA | NA | NA | NA | NA |
| WCM213_Z13_1 | 6 | 99572563 | 118886989 | −0.5112 | NA | NA | NA | NA | NA |
| WCM213_Z13_1 | 6 | 118887303 | 170893407 | −0.2006 | NA | NA | NA | NA | NA |
| WCM213_Z13_1 | 7 | 193451 | 37136228 | −0.0214 | NA | NA | NA | NA | NA |
| WCM213_Z13_1 | 7 | 37172789 | 37903060 | 0.5554 | NA | NA | NA | NA | NA |
| WCM213_Z13_1 | 7 | 37905208 | 45108060 | 0.4152 | NA | NA | NA | NA | NA |
| WCM213_Z13_1 | 7 | 45109511 | 81335063 | 0.5599 | NA | NA | NA | NA | NA |
| WCM213_Z13_1 | 7 | 81335673 | 99375666 | 0.2733 | NA | NA | NA | NA | NA |
| WCM213_Z13_1 | 7 | 99377661 | 110526661 | 0.4698 | NA | NA | NA | NA | NA |
| WCM213_Z13_1 | 7 | 110603137 | 158937375 | 0.3 | NA | NA | NA | NA | NA |
| WCM213_Z13_1 | 8 | 190908 | 22675196 | −0.6668 | NA | NA | NA | NA | NA |
| WCM213_Z13_1 | 8 | 22777766 | 37333471 | −0.4215 | NA | NA | NA | NA | NA |
| WCM213_Z13_1 | 8 | 37374128 | 38775571 | 0.5906 | NA | NA | NA | NA | NA |
| WCM213_Z13_1 | 8 | 38793527 | 43157113 | 0.1777 | NA | NA | NA | NA | NA |
| WCM213_Z13_1 | 8 | 43171038 | 50449788 | −0.1825 | NA | NA | NA | NA | NA |
| WCM213_Z13_1 | 8 | 51306813 | 64100298 | 0.3216 | NA | NA | NA | NA | NA |
| WCM213_Z13_1 | 8 | 65286010 | 67547149 | 0.9455 | NA | NA | NA | NA | NA |
| WCM213_Z13_1 | 8 | 67547257 | 77896016 | 0.6605 | NA | NA | NA | NA | NA |
| WCM213_Z13_1 | 8 | 77896321 | 89340194 | 0.9933 | NA | NA | NA | NA | NA |
| WCM213_Z13_1 | 8 | 90770338 | 99468115 | 0.4532 | NA | NA | NA | NA | NA |
| WCM213_Z13_1 | 8 | 99539049 | 102731537 | 0.0455 | NA | NA | NA | NA | NA |
| WCM213_Z13_1 | 8 | 102731825 | 103662579 | 0.8502 | NA | NA | NA | NA | NA |
| WCM213_Z13_1 | 8 | 103663391 | 142264209 | 0.4141 | NA | NA | NA | NA | NA |
| WCM213_Z13_1 | 8 | 142264797 | 146279480 | 0.5117 | NA | NA | NA | NA | NA |
| WCM213_Z13_1 | 9 | 17056 | 27055662 | −0.106 | NA | NA | NA | NA | NA |
| WCM213_Z13_1 | 9 | 27056372 | 69256883 | 0.1551 | NA | NA | NA | NA | NA |
| WCM213_Z13_1 | 9 | 71144547 | 113169030 | −0.3184 | NA | NA | NA | NA | NA |
| WCM213_Z13_1 | 9 | 113169442 | 132382629 | −0.2021 | NA | NA | NA | NA | NA |
| WCM213_Z13_1 | 9 | 132395015 | 133769178 | −0.2765 | NA | NA | NA | NA | NA |
| WCM213_Z13_1 | 9 | 133779422 | 141111414 | −0.0939 | NA | NA | NA | NA | NA |
| WCM213_Z13_1 | X | 2700119 | 67431965 | 0.3733 | NA | NA | NA | NA | NA |
| WCM213_Z13_1 | X | 67454405 | 154509348 | −0.2057 | NA | NA | NA | NA | NA |
| WCM216 | 1 | 14930 | 899359 | −0.0562 | −0.1679 | 0.9533 | 0.827 | 1 | 1 |
| WCM216 | 1 | 899571 | 35577385 | 0.0817 | 0.1216 | 1.0988 | 1.0771 | 1 | 1 |
| WCM216 | 1 | 35577746 | 249213961 | 0.0023 | −0.0413 | 0.9718 | 0.9718 | 1 | 1 |
| WCM216 | 10 | 225958 | 135438909 | 0.0245 | 0.0053 | 1.0037 | 1.0037 | 1 | 1 |
| WCM216 | 11 | 128378 | 134605827 | 0.0143 | −0.0161 | 0.9889 | 0.9889 | 1 | 1 |
| WCM216 | 12 | 148567 | 133810704 | 0.0103 | −0.0244 | 0.9832 | 0.9832 | 1 | 1 |
| WCM216 | 13 | 19042165 | 115089411 | −0.0019 | −0.0502 | 0.9658 | 0.9658 | 1 | 1 |
| WCM216 | 14 | 19110174 | 107283270 | 0.0016 | −0.0428 | 0.9708 | 0.9708 | 1 | 1 |
| WCM216 | 15 | 20083972 | 102506257 | 0.03 | 0.0167 | 1.0116 | 1.0116 | 1 | 1 |
| WCM216 | 16 | 83338 | 90168857 | 0.015 | −0.0147 | 0.9899 | 0.9899 | 1 | 1 |
| WCM216 | 17 | 131595 | 81052551 | 0.0218 | −4.00E−04 | 0.9998 | 0.9998 | 1 | 1 |
| WCM216 | 18 | 47588 | 78005115 | 0.0035 | −0.0387 | 0.9735 | 0.9735 | 1 | 1 |
| WCM216 | 19 | 105021 | 59093696 | 0.0115 | −0.0221 | 0.9848 | 0.9848 | 1 | 1 |
| WCM216 | 2 | 41680 | 243037022 | −0.0013 | −0.0489 | 0.9666 | 0.9666 | 1 | 1 |
| WCM216 | 20 | 68406 | 62934541 | 0.0527 | 0.0633 | 1.0448 | 1.0448 | 1 | 1 |
| WCM216 | 21 | 9590305 | 41559253 | −0.0469 | −0.1474 | 0.9168 | 0.889 | 1 | 1 |
| WCM216 | 21 | 41559927 | 47700514 | 0.1239 | 0.2047 | 1.1524 | 1.1524 | 1 | 1 |
| WCM216 | 21 | 47703649 | 48084207 | 0.0062 | −0.033 | 1.0639 | 0.8908 | 1 | 1 |
| WCM216 | 22 | 16159043 | 51223730 | 0.0746 | 0.1076 | 1.0774 | 1.0774 | 1 | 1 |
| WCM216 | 3 | 239347 | 197896705 | 0.0049 | −0.0359 | 0.9754 | 0.9754 | 1 | 1 |
| WCM216 | 4 | 53349 | 190882003 | −0.0254 | −0.1007 | 0.9326 | 0.9326 | 1 | 1 |
| WCM216 | 5 | 90750 | 180687828 | −0.0123 | −0.0724 | 0.951 | 0.951 | 1 | 1 |
| WCM216 | 6 | 203561 | 170893411 | −0.0032 | −0.0531 | 0.9639 | 0.9639 | 1 | 1 |
| WCM216 | 7 | 193189 | 158937375 | 0.0163 | −0.0119 | 0.998 | 0.9856 | 1 | 1 |
| WCM216 | 8 | 163550 | 146279514 | 0.0062 | −0.0332 | 0.9773 | 0.9773 | 1 | 1 |
| WCM216 | 9 | 14822 | 111625219 | −0.0143 | −0.0767 | 0.9482 | 0.9482 | 1 | 1 |
| WCM216 | 9 | 111625520 | 141015379 | 0.0775 | 0.1133 | 1.0817 | 1.0817 | 1 | 1 |
| WCM216 | 9 | 141015921 | 141107533 | −0.1788 | −0.4553 | 1.458 | 6.00E−04 | 1 | 0 |
| WCM216 | X | 2700202 | 154493589 | −0.0648 | −0.1872 | NA | NA | NA | NA |
| WCM216_Z1_1 | 1 | 69569 | 249212346 | −0.006 | NA | NA | NA | NA | NA |
| WCM216_Z1_1 | 10 | 226016 | 135440108 | −0.0158 | NA | NA | NA | NA | NA |
| WCM216_Z1_1 | 11 | 193152 | 134856597 | −0.0069 | NA | NA | NA | NA | NA |
| WCM216_Z1_1 | 12 | 148674 | 133810703 | −0.0061 | NA | NA | NA | NA | NA |
| WCM216_Z1_1 | 13 | 19600475 | 115089411 | −0.0367 | NA | NA | NA | NA | NA |
| WCM216_Z1_1 | 14 | 19377703 | 107283027 | −0.0054 | NA | NA | NA | NA | NA |
| WCM216_Z1_1 | 15 | 20170029 | 102506273 | −0.0127 | NA | NA | NA | NA | NA |
| WCM216_Z1_1 | 16 | 97531 | 90162166 | 0.0013 | NA | NA | NA | NA | NA |
| WCM216_Z1_1 | 17 | 131595 | 81083455 | 0.0052 | NA | NA | NA | NA | NA |
| WCM216_Z1_1 | 18 | 48269 | 78005197 | −0.0417 | NA | NA | NA | NA | NA |
| WCM216_Z1_1 | 19 | 281424 | 59083929 | 0.0086 | NA | NA | NA | NA | NA |
| WCM216_Z1_1 | 2 | 41613 | 242946607 | −0.0089 | NA | NA | NA | NA | NA |
| WCM216_Z1_1 | 20 | 68351 | 62904863 | −0.0019 | NA | NA | NA | NA | NA |
| WCM216_Z1_1 | 21 | 9826207 | 48084281 | −6.00E−04 | NA | NA | NA | NA | NA |
| WCM216_Z1_1 | 22 | 16287480 | 51219138 | 0.0065 | NA | NA | NA | NA | NA |
| WCM216_Z1_1 | 3 | 239443 | 197896720 | −0.0057 | NA | NA | NA | NA | NA |
| WCM216_Z1_1 | 4 | 59331 | 190426502 | −0.0268 | NA | NA | NA | NA | NA |
| WCM216_Z1_1 | 5 | 140532 | 180687803 | −0.017 | NA | NA | NA | NA | NA |
| WCM216_Z1_1 | 6 | 203510 | 170893407 | −0.0179 | NA | NA | NA | NA | NA |
| WCM216_Z1_1 | 7 | 193265 | 158937185 | −0.0118 | NA | NA | NA | NA | NA |
| WCM216_Z1_1 | 8 | 190907 | 61482596 | −0.0488 | NA | NA | NA | NA | NA |
| WCM216_Z1_1 | 8 | 61484612 | 135621053 | 0.0357 | NA | NA | NA | NA | NA |
| WCM216_Z1_1 | 8 | 135622763 | 146279544 | −0.0539 | NA | NA | NA | NA | NA |
| WCM216_Z1_1 | 9 | 17066 | 141111414 | −0.0144 | NA | NA | NA | NA | NA |
| WCM216_Z1_1 | X | 2700147 | 154509348 | −0.0478 | NA | NA | NA | NA | NA |
| WCM218 | 1 | 14930 | 249213964 | 0.0263 | 0.1425 | 1.1038 | 1.1038 | 1 | 1 |
| WCM218 | 10 | 226116 | 135477891 | 0.0176 | 0.0797 | 1.0568 | 1.0568 | 1 | 1 |
| WCM218 | 11 | 193135 | 62300042 | 0.0336 | 0.1927 | 1.2369 | 1.0489 | 1 | 1 |
| WCM218 | 11 | 62300434 | 66445317 | −0.129 | −1.6995 | 0.3919 | 0.2239 | 0 | 0 |
| WCM218 | 11 | 66453322 | 134605959 | 0.0242 | 0.127 | 1.092 | 1.092 | 1 | 1 |
| WCM218 | 12 | 148513 | 133810769 | 0.0067 | −0.0026 | 0.9982 | 0.9982 | 1 | 1 |
| WCM218 | 13 | 19041965 | 115089411 | −6.00E−04 | −0.0598 | 0.9594 | 0.9594 | 1 | 1 |
| WCM218 | 14 | 19110251 | 107283270 | 0.0069 | −8.00E−04 | 0.9994 | 0.9994 | 1 | 1 |
| WCM218 | 15 | 20083896 | 102514188 | 0.0308 | 0.1736 | 1.1278 | 1.1278 | 1 | 1 |
| WCM218 | 16 | 67591 | 28931150 | 0.0152 | 0.0622 | 1.044 | 1.044 | 1 | 1 |
| WCM218 | 16 | 28935787 | 46726503 | −0.1581 | −2.5703 | 0.3536 | −0.0169 | 0 | 0 |
| WCM218 | 16 | 46727031 | 90260461 | 0.0088 | 0.0136 | 1.0094 | 1.0094 | 1 | 1 |
| WCM218 | 17 | 131595 | 81187946 | 8.00E−04 | −0.0486 | 0.9669 | 0.9669 | 1 | 1 |
| WCM218 | 18 | 47654 | 78005197 | 0.0076 | 0.005 | 1.0034 | 1.0034 | 1 | 1 |
| WCM218 | 19 | 105050 | 59093645 | −0.0524 | −0.5361 | 0.6896 | 0.6896 | 1 | 1 |
| WCM218 | 2 | 41613 | 243037022 | 0.0068 | −0.0013 | 0.9991 | 0.9991 | 1 | 1 |
| WCM218 | 20 | 68446 | 62934572 | 0.0357 | 0.2071 | 1.1543 | 1.1543 | 1 | 1 |
| WCM218 | 21 | 9590311 | 48084214 | 0.0093 | 0.0173 | 1.0121 | 1.0121 | 1 | 1 |
| WCM218 | 22 | 16159021 | 51223730 | 0.0322 | 0.1833 | 1.1355 | 1.1355 | 1 | 1 |
| WCM218 | 3 | 239451 | 197896714 | 0.0094 | 0.0183 | 1.0128 | 1.0128 | 1 | 1 |
| WCM218 | 4 | 53396 | 190903980 | 0.0146 | 0.0571 | 1.0404 | 1.0404 | 1 | 1 |
| WCM218 | 5 | 90751 | 180687804 | −0.0014 | −0.0661 | 0.9552 | 0.9552 | 1 | 1 |
| WCM218 | 6 | 203397 | 170892918 | −3.00E−04 | −0.0571 | 0.9612 | 0.9612 | 1 | 1 |
| WCM218 | 7 | 193205 | 158937011 | 0.012 | 0.038 | 1.0267 | 1.0267 | 1 | 1 |
| WCM218 | 8 | 163476 | 146279529 | 0.0089 | 0.0146 | 1.0102 | 1.0102 | 1 | 1 |
| WCM218 | 9 | 15879 | 141107515 | 0.0323 | 0.1843 | 1.1363 | 1.1363 | 1 | 1 |
| WCM218 | X | 2700163 | 154493601 | −0.0584 | −0.6014 | NA | NA | NA | NA |
| WCM218_Z11_1 | 1 | 69592 | 31187094 | −0.4185 | −1.0514 | 1.0263 | −0.0613 | 1 | 0 |
| WCM218_Z11_1 | 1 | 31188089 | 145747046 | 0.0081 | 0.0962 | 1.085 | 1.0529 | 1 | 1 |
| WCM218_Z11_1 | 1 | 145748529 | 158605722 | 0.2868 | 0.639 | 2.0548 | 1.0597 | 2 | 1 |
| WCM218_Z11_1 | 1 | 158606463 | 205770144 | 0.2566 | 0.5846 | 1.9678 | 1.0315 | 2 | 1 |
| WCM218_Z11_1 | 1 | 205779231 | 227921639 | 0.2583 | 0.5877 | 2.2086 | 0.797 | 2 | 1 |
| WCM218_Z11_1 | 1 | 227922517 | 228363152 | −0.0943 | −0.1329 | NA | NA | NA | NA |
| WCM218_Z11_1 | 1 | 228391808 | 249212241 | 0.0985 | 0.2834 | 2.4709 | −0.0367 | 2 | 0 |
| WCM218_Z11_1 | 10 | 225981 | 23409768 | −0.3891 | −0.9503 | 0.9785 | 0.0565 | 1 | 0 |
| WCM218_Z11_1 | 10 | 23481591 | 75898098 | 0.0365 | 0.1565 | 1.3296 | 0.8996 | 1 | 1 |
| WCM218_Z11_1 | 10 | 75911036 | 135440081 | −0.4467 | −1.1536 | 0.9377 | −0.0386 | 1 | 0 |
| WCM218_Z11_1 | 11 | 193102 | 134856536 | −0.0601 | −0.0541 | 0.9807 | 0.9457 | 1 | 1 |
| WCM218_Z11_1 | 12 | 148673 | 60162031 | −0.0777 | −0.0943 | 0.9648 | 0.9087 | 1 | 1 |
| WCM218_Z11_1 | 12 | 60165031 | 133810896 | 0.22 | 0.5174 | 1.925 | 0.9378 | 2 | 1 |
| WCM218_Z11_1 | 13 | 19600515 | 19755886 | −0.4702 | −1.243 | NA | NA | NA | NA |
| WCM218_Z11_1 | 13 | 19759475 | 37679268 | −0.1453 | −0.2551 | 0.9162 | 0.7596 | 1 | 1 |
| WCM218_Z11_1 | 13 | 37804353 | 96261756 | −0.3747 | −0.9024 | 0.9954 | 0.0746 | 1 | 0 |
| WCM218_Z11_1 | 13 | 96263699 | 115089411 | 0.5032 | 1.0051 | 2.9404 | 1.0738 | 3 | 1 |
| WCM218_Z11_1 | 14 | 19377711 | 107283047 | −0.0148 | 0.0466 | 1.0509 | 1.0148 | 1 | 1 |
| WCM218_Z11_1 | 15 | 20170150 | 102514154 | 0.0024 | 0.084 | 1.0828 | 1.0372 | 1 | 1 |
| WCM218_Z11_1 | 16 | 67604 | 68725783 | −0.003 | 0.0723 | 1.0673 | 1.0355 | 1 | 1 |
| WCM218_Z11_1 | 16 | 68729264 | 68835716 | −2.2104 | NA | NA | NA | NA | NA |
| WCM218_Z11_1 | 16 | 68842395 | 79619821 | 8.00E−04 | 0.0805 | 1.0824 | 1.0323 | 1 | 1 |
| WCM218_Z11_1 | 16 | 79628419 | 90108931 | −0.3757 | −0.9057 | 1.0448 | 0.0228 | 1 | 0 |
| WCM218_Z11_1 | 16 | 90109662 | 90161584 | −0.1172 | −0.187 | NA | NA | NA | NA |
| WCM218_Z11_1 | 17 | 131595 | 13977759 | −0.343 | −0.8007 | 1.0662 | 0.0819 | 1 | 0 |
| WCM218_Z11_1 | 17 | 13980240 | 30771558 | 0.0014 | 0.0819 | 1.0995 | 1.0174 | 1 | 1 |
| WCM218_Z11_1 | 17 | 30774004 | 41961452 | 0.2828 | 0.6318 | 2.0429 | 1.0562 | 2 | 1 |
| WCM218_Z11_1 | 17 | 41975745 | 42829321 | −0.3464 | −0.8114 | NA | NA | NA | NA |
| WCM218_Z11_1 | 17 | 42837130 | 42855082 | 0.4011 | 0.837 | NA | NA | NA | NA |
| WCM218_Z11_1 | 17 | 42855228 | 79099738 | 0.345 | 0.7414 | 2.2158 | 1.1277 | 2 | 1 |
| WCM218_Z11_1 | 17 | 79100235 | 80992962 | 0.5769 | 1.1218 | 2.234 | 2.1184 | 2 | 2 |
| WCM218_Z11_1 | 17 | 81006387 | 81083419 | 0.7421 | 1.3721 | NA | NA | NA | NA |
| WCM218_Z11_1 | 18 | 48221 | 78005215 | −0.0568 | −0.0467 | 0.9966 | 0.9397 | 1 | 1 |
| WCM218_Z11_1 | 19 | 281404 | 7795248 | −0.3702 | −0.8876 | 1.131 | −0.05 | 1 | 0 |
| WCM218_Z11_1 | 19 | 7795940 | 47589710 | −0.0216 | 0.0319 | 1.0448 | 0.9999 | 1 | 1 |
| WCM218_Z11_1 | 19 | 47593410 | 49004797 | −0.3788 | −0.916 | 1.0539 | 0.0061 | 1 | 0 |
| WCM218_Z11_1 | 19 | 49005758 | 59083968 | −0.0169 | 0.0421 | 1.0544 | 1.0048 | 1 | 1 |
| WCM218_Z11_1 | 2 | 41615 | 272203 | 0.0891 | 0.2645 | NA | NA | NA | NA |
| WCM218_Z11_1 | 2 | 275167 | 234763031 | −0.0134 | 0.0498 | 1.0509 | 1.0193 | 1 | 1 |
| WCM218_Z11_1 | 2 | 234774887 | 242946597 | −0.3842 | −0.934 | 1.0227 | 0.0242 | 1 | 0 |
| WCM218_Z11_1 | 20 | 68351 | 62904848 | 2.00E−04 | 0.0792 | 1.0792 | 1.0336 | 1 | 1 |
| WCM218_Z11_1 | 21 | 9826225 | 48084210 | −0.0121 | 0.0527 | 1.0688 | 1.0056 | 1 | 1 |
| WCM218_Z11_1 | 22 | 17060359 | 51215120 | −0.0154 | 0.0453 | 1.0567 | 1.0071 | 1 | 1 |
| WCM218_Z11_1 | 3 | 239451 | 33907993 | −0.2561 | −0.5446 | 0.9755 | 0.3956 | 1 | 0 |
| WCM218_Z11_1 | 3 | 35723322 | 190106199 | 0.0186 | 0.1186 | 1.0857 | 1.0857 | 1 | 1 |
| WCM218_Z11_1 | 3 | 190120142 | 197894712 | −0.2846 | −0.6255 | 1.0746 | 0.2217 | 1 | 0 |
| WCM218_Z11_1 | 4 | 59361 | 100479298 | −0.0604 | −0.0548 | 0.9802 | 0.9453 | 1 | 1 |
| WCM218_Z11_1 | 4 | 100480481 | 187542217 | 0.2107 | 0.5002 | 1.8336 | 0.9952 | 2 | 1 |
| WCM218_Z11_1 | 4 | 187542394 | 190426459 | 0.165 | 0.4137 | NA | NA | NA | NA |
| WCM218_Z11_1 | 5 | 140663 | 148610209 | −0.0728 | −0.0831 | 0.9614 | 0.9267 | 1 | 1 |
| WCM218_Z11_1 | 5 | 148612820 | 168096886 | 0.2079 | 0.495 | 1.8777 | 0.9409 | 2 | 1 |
| WCM218_Z11_1 | 5 | 168098235 | 168195160 | 0.2488 | 0.5704 | NA | NA | NA | NA |
| WCM218_Z11_1 | 5 | 168199876 | 180622668 | −0.0497 | −0.0306 | 2.0048 | −0.0468 | 2 | 0 |
| WCM218_Z11_1 | 5 | 180625186 | 180687808 | 0.0941 | 0.2747 | NA | NA | NA | NA |
| WCM218_Z11_1 | 6 | 203695 | 12122818 | −0.4402 | −1.1296 | 0.8164 | 0.0977 | 1 | 0 |
| WCM218_Z11_1 | 6 | 12123218 | 64356570 | −0.0556 | −0.044 | 1.0051 | 0.9349 | 1 | 1 |
| WCM218_Z11_1 | 6 | 64390027 | 65300775 | −0.4674 | −1.2323 | NA | NA | NA | NA |
| WCM218_Z11_1 | 6 | 65301167 | 93956647 | −0.3915 | −0.9585 | 1.0409 | −0.0117 | 1 | 0 |
| WCM218_Z11_1 | 6 | 93965751 | 170893407 | −0.0596 | −0.053 | 0.9902 | 0.9377 | 1 | 1 |
| WCM218_Z11_1 | 7 | 193245 | 129765724 | −0.0584 | −0.0503 | 1.1517 | 0.7798 | 1 | 1 |
| WCM218_Z11_1 | 7 | 129769310 | 158937018 | 0.3952 | 0.827 | 2.0108 | 1.5373 | 2 | 2 |
| WCM218_Z11_1 | 8 | 190897 | 280879 | 0.4834 | 0.9731 | NA | NA | NA | NA |
| WCM218_Z11_1 | 8 | 328513 | 19276220 | −0.0416 | −0.0124 | 2.0938 | −0.111 | 2 | 0 |
| WCM218_Z11_1 | 8 | 19277858 | 35563468 | −0.4454 | −1.149 | 0.8859 | 0.0159 | 1 | 0 |
| WCM218_Z11_1 | 8 | 35579745 | 67352400 | −0.0979 | −0.1412 | 0.977 | 0.8365 | 1 | 1 |
| WCM218_Z11_1 | 8 | 67355054 | 146112284 | 0.2144 | 0.507 | 1.9392 | 0.903 | 2 | 1 |
| WCM218_Z11_1 | 8 | 146115037 | 146279529 | 0.0523 | 0.1893 | NA | NA | NA | NA |
| WCM218_Z11_1 | 9 | 17058 | 8518143 | −0.3488 | −0.8188 | 1.041 | 0.0928 | 1 | 0 |
| WCM218_Z11_1 | 9 | 8518395 | 38527602 | −0.0743 | −0.0866 | 0.9872 | 0.8963 | 1 | 1 |
| WCM218_Z11_1 | 9 | 38527860 | 141111414 | 0.1734 | 0.4298 | 1.7813 | 0.9128 | 2 | 1 |
| WCM218_Z11_1 | X | 2707711 | 65819596 | −0.105 | −0.1581 | NA | NA | NA | NA |
| WCM218_Z11_1 | X | 65822515 | 76938748 | 0.4655 | 0.9439 | NA | NA | NA | NA |
| WCM218_Z11_1 | X | 76939226 | 154509348 | −0.1015 | −0.1499 | NA | NA | NA | NA |
| WCM218_Z3_1 | 1 | 69592 | 31186434 | −0.4433 | −0.8552 | 1.045 | 0.0606 | 1 | 0 |
| WCM218_Z3_1 | 1 | 31187146 | 146232669 | −0.0344 | 0.0029 | 1.0111 | 0.9929 | 1 | 1 |
| WCM218_Z3_1 | 1 | 146398506 | 227348252 | 0.3067 | 0.573 | 1.9856 | 0.9895 | 2 | 1 |
| WCM218_Z3_1 | 1 | 227381561 | 228362451 | 0.0177 | 0.0962 | NA | NA | NA | NA |
| WCM218_Z3_1 | 1 | 228362682 | 243809321 | −0.0587 | −0.0416 | 1.9846 | −0.0414 | 2 | 0 |
| WCM218_Z3_1 | 1 | 243858900 | 249212243 | −0.4994 | −0.9987 | 1.0371 | −0.0362 | 1 | 0 |
| WCM218_Z3_1 | 10 | 225952 | 3827207 | −0.0655 | −0.0541 | 0.9899 | 0.9365 | 1 | 1 |
| WCM218_Z3_1 | 10 | 4867218 | 23303037 | −0.5063 | −1.0167 | 0.989 | −5.00E−04 | 1 | 0 |
| WCM218_Z3_1 | 10 | 23319547 | 63441055 | −0.0728 | −0.0675 | 0.9879 | 0.9206 | 1 | 1 |
| WCM218_Z3_1 | 10 | 63445812 | 75863657 | 0.1074 | 0.251 | 1.2282 | 1.1518 | 1 | 1 |
| WCM218_Z3_1 | 10 | 75865002 | 76158233 | −0.0925 | −0.1045 | NA | NA | NA | NA |
| WCM218_Z3_1 | 10 | 76285109 | 135440068 | −0.4905 | −0.9751 | 1.0116 | 0.0058 | 1 | 0 |
| WCM218_Z3_1 | 11 | 193127 | 66481170 | −0.041 | −0.0092 | 1.0136 | 0.9737 | 1 | 1 |
| WCM218_Z3_1 | 11 | 66481812 | 69469088 | 0.3875 | 0.6962 | 2.1031 | 1.1374 | 2 | 1 |
| WCM218_Z3_1 | 11 | 69469211 | 134856542 | −0.0416 | −0.0103 | 1.0183 | 0.9676 | 1 | 1 |
| WCM218_Z3_1 | 12 | 148642 | 60173356 | −0.0427 | −0.0123 | 1.0657 | 0.9173 | 1 | 1 |
| WCM218_Z3_1 | 12 | 62104199 | 133810869 | 0.3035 | 0.5679 | 1.9816 | 0.9832 | 2 | 1 |
| WCM218_Z3_1 | 13 | 19600371 | 32366149 | −0.0398 | −0.0068 | 1.2183 | 0.7722 | 1 | 1 |
| WCM218_Z3_1 | 13 | 32367046 | 34395385 | −0.7409 | −1.7505 | NA | NA | NA | NA |
| WCM218_Z3_1 | 13 | 34398071 | 38145574 | −0.1893 | −0.2921 | NA | NA | NA | NA |
| WCM218_Z3_1 | 13 | 38151931 | 96264374 | −0.5075 | −1.0199 | 0.9757 | 0.0106 | 1 | 0 |
| WCM218_Z3_1 | 13 | 96272196 | 115089411 | 0.4104 | 0.7306 | 2.2638 | 1.0548 | 2 | 1 |
| WCM218_Z3_1 | 14 | 19377727 | 75138157 | −0.0482 | −0.0222 | 1.019 | 0.9504 | 1 | 1 |
| WCM218_Z3_1 | 14 | 75139811 | 75421875 | −0.4981 | −0.995 | NA | NA | NA | NA |
| WCM218_Z3_1 | 14 | 75469723 | 107283210 | −0.0305 | 0.01 | 1.0289 | 0.9851 | 1 | 1 |
| WCM218_Z3_1 | 15 | 20170150 | 102514154 | −0.0384 | −0.0043 | 1.0206 | 0.9734 | 1 | 1 |
| WCM218_Z3_1 | 16 | 67604 | 79245956 | −0.0208 | 0.0274 | 1.0339 | 1.0045 | 1 | 1 |
| WCM218_Z3_1 | 16 | 79619821 | 90233627 | −0.4618 | −0.9016 | 1.0483 | 0.0223 | 1 | 0 |
| WCM218_Z3_1 | 17 | 131595 | 13400048 | −0.4783 | −0.9435 | 0.9803 | 0.0596 | 1 | 0 |
| WCM218_Z3_1 | 17 | 13446846 | 30202424 | −0.0106 | 0.0458 | 1.1322 | 0.9324 | 1 | 1 |
| WCM218_Z3_1 | 17 | 30205278 | 41961469 | 0.3249 | 0.6011 | 1.9605 | 1.0732 | 2 | 1 |
| WCM218_Z3_1 | 17 | 41975714 | 42837180 | −0.4728 | −0.9294 | NA | NA | NA | NA |
| WCM218_Z3_1 | 17 | 42847102 | 79106464 | 0.3285 | 0.6066 | 2.0517 | 0.9937 | 2 | 1 |
| WCM218_Z3_1 | 17 | 79107061 | 81083455 | 0.6407 | 1.0622 | 2.1317 | 2.0445 | 2 | 2 |
| WCM218_Z3_1 | 18 | 48266 | 67992954 | −0.0516 | −0.0284 | 0.9895 | 0.9715 | 1 | 1 |
| WCM218_Z3_1 | 18 | 67993232 | 78005206 | −0.466 | −0.9122 | 1.0037 | 0.059 | 1 | 0 |
| WCM218_Z3_1 | 19 | 281483 | 7830099 | −0.4535 | −0.8807 | 1.0604 | 0.0258 | 1 | 0 |
| WCM218_Z3_1 | 19 | 7830571 | 47597859 | −0.0258 | 0.0185 | 1.0294 | 0.9965 | 1 | 1 |
| WCM218_Z3_1 | 19 | 47615692 | 49001956 | −0.4852 | −0.9614 | 1.0223 | 0.0048 | 1 | 0 |
| WCM218_Z3_1 | 19 | 49002413 | 59083953 | −0.0292 | 0.0123 | 1.0177 | 0.9995 | 1 | 1 |
| WCM218_Z3_1 | 2 | 41609 | 73989806 | −0.0434 | −0.0136 | 1.0124 | 0.9689 | 1 | 1 |
| WCM218_Z3_1 | 2 | 73993650 | 74466483 | −0.4953 | −0.9877 | NA | NA | NA | NA |
| WCM218_Z3_1 | 2 | 74469159 | 160604812 | −0.0523 | −0.0298 | 0.9957 | 0.9634 | 1 | 1 |
| WCM218_Z3_1 | 2 | 160605188 | 161349869 | −0.5083 | −1.0221 | NA | NA | NA | NA |
| WCM218_Z3_1 | 2 | 161797513 | 234839347 | −0.0625 | −0.0485 | 0.9991 | 0.9348 | 1 | 1 |
| WCM218_Z3_1 | 2 | 234846108 | 242946604 | −0.4873 | −0.9669 | 0.997 | 0.0262 | 1 | 0 |
| WCM218_Z3_1 | 20 | 68396 | 62904847 | −0.0299 | 0.0111 | 1.0151 | 1.0004 | 1 | 1 |
| WCM218_Z3_1 | 21 | 9826203 | 48084277 | −0.0405 | −0.0081 | 1.0053 | 0.9835 | 1 | 1 |
| WCM218_Z3_1 | 22 | 16287481 | 51215137 | −0.0227 | 0.0241 | 1.0278 | 1.0058 | 1 | 1 |
| WCM218_Z3_1 | 3 | 239350 | 197894713 | −0.0417 | −0.0104 | 1.0091 | 0.9765 | 1 | 1 |
| WCM218_Z3_1 | 4 | 59375 | 367639 | 0.131 | 0.2906 | NA | NA | NA | NA |
| WCM218_Z3_1 | 4 | 420497 | 100575735 | −0.06 | −0.0439 | 0.9789 | 0.9611 | 1 | 1 |
| WCM218_Z3_1 | 4 | 100738152 | 190426459 | 0.2743 | 0.5224 | 1.9188 | 0.9539 | 2 | 1 |
| WCM218_Z3_1 | 5 | 140684 | 148521664 | −0.0491 | −0.0239 | 0.998 | 0.9691 | 1 | 1 |
| WCM218_Z3_1 | 5 | 148563110 | 169127097 | 0.2827 | 0.5355 | 1.937 | 0.9619 | 2 | 1 |
| WCM218_Z3_1 | 5 | 169129414 | 180687814 | −0.0383 | −0.0041 | 1.9883 | 0.006 | 2 | 0 |
| WCM218_Z3_1 | 6 | 203545 | 12089436 | −0.2821 | −0.4843 | 0.9632 | 0.4665 | 1 | 0 |
| WCM218_Z3_1 | 6 | 12120127 | 58250780 | −0.0412 | −0.0095 | 1.0134 | 0.9735 | 1 | 1 |
| WCM218_Z3_1 | 6 | 62284302 | 62757815 | −0.2277 | −0.3699 | NA | NA | NA | NA |
| WCM218_Z3_1 | 6 | 62887108 | 93969182 | −0.5103 | −1.0275 | 0.9735 | 0.0076 | 1 | 0 |
| WCM218_Z3_1 | 6 | 93973575 | 170893408 | −0.0646 | −0.0524 | 0.9821 | 0.9465 | 1 | 1 |
| WCM218_Z3_1 | 7 | 193265 | 127031567 | −0.0394 | −0.0062 | 1.0175 | 0.9739 | 1 | 1 |
| WCM218_Z3_1 | 7 | 127222157 | 139026260 | 0.289 | 0.5453 | 1.974 | 0.9446 | 2 | 1 |
| WCM218_Z3_1 | 7 | 139030252 | 158937022 | 0.5603 | 0.949 | 1.9608 | 1.9003 | 2 | 2 |
| WCM218_Z3_1 | 8 | 190906 | 328513 | 0.0979 | 0.235 | NA | NA | NA | NA |
| WCM218_Z3_1 | 8 | 328616 | 19456843 | 0.0171 | 0.0951 | 2.164 | −0.0278 | 2 | 0 |
| WCM218_Z3_1 | 8 | 19675096 | 36644853 | −0.4757 | −0.9369 | 1.0532 | −0.0085 | 1 | 0 |
| WCM218_Z3_1 | 8 | 36661556 | 67422385 | −0.0524 | −0.03 | 1.0549 | 0.9039 | 1 | 1 |
| WCM218_Z3_1 | 8 | 67425832 | 146279544 | 0.3003 | 0.5629 | 1.934 | 1.0204 | 2 | 1 |
| WCM218_Z3_1 | 9 | 17085 | 12695819 | −0.5077 | −1.0204 | 0.9833 | 0.0026 | 1 | 0 |
| WCM218_Z3_1 | 9 | 12698568 | 71155663 | −0.0579 | −0.0401 | 1.0174 | 0.9278 | 1 | 1 |
| WCM218_Z3_1 | 9 | 71395275 | 141111414 | 0.2949 | 0.5545 | 1.926 | 1.0113 | 2 | 1 |
| WCM218_Z3_1 | X | 2707708 | 65486503 | −0.1034 | −0.1251 | NA | NA | NA | NA |
| WCM218_Z3_1 | X | 65819332 | 78622701 | 0.5402 | 0.9204 | NA | NA | NA | NA |
| WCM218_Z3_1 | X | 79277892 | 154509348 | −0.1042 | −0.1265 | NA | NA | NA | NA |
| WCM219 | 1 | 14896 | 249213458 | 0.0217 | 0.1472 | 1.1878 | 1.027 | 1 | 1 |
| WCM219 | 10 | 225960 | 135439016 | 0.016 | 0.0389 | 1.0874 | 0.9672 | 1 | 1 |
| WCM219 | 11 | 193096 | 134605906 | 0.0173 | 0.0646 | 2.1884 | −0.0968 | 2 | 0 |
| WCM219 | 12 | 148513 | 133810703 | 0.0072 | −0.1415 | 0.9663 | 0.8468 | 1 | 1 |
| WCM219 | 13 | 19041994 | 115089411 | 0.0034 | −0.2289 | 0.8731 | 0.8334 | 1 | 1 |
| WCM219 | 14 | 19110183 | 107283266 | 0.0164 | 0.048 | 1.114 | 0.9537 | 1 | 1 |
| WCM219 | 15 | 20083896 | 102516227 | 0.0346 | 0.3654 | 1.3897 | 1.1868 | 1 | 1 |
| WCM219 | 16 | 64400 | 90260461 | 0.0082 | −0.1196 | 0.9802 | 0.8607 | 1 | 1 |
| WCM219 | 17 | 131595 | 81187855 | 0.0159 | 0.0368 | 1.0259 | 1.0259 | 1 | 1 |
| WCM219 | 18 | 47581 | 78005279 | 0.0091 | −0.1005 | 1.0124 | 0.853 | 1 | 1 |
| WCM219 | 19 | 105010 | 59093696 | −0.0095 | −0.5645 | 0.7549 | 0.5975 | 1 | 1 |
| WCM219 | 2 | 41595 | 243037022 | 0.0059 | −0.1711 | 0.8882 | 0.8882 | 1 | 1 |
| WCM219 | 20 | 68340 | 62934592 | 0.0399 | 0.4459 | 1.464 | 1.2603 | 1 | 1 |
| WCM219 | 21 | 9590311 | 48084170 | 0.0208 | 0.1294 | 1.134 | 1.0536 | 1 | 1 |
| WCM219 | 22 | 16159068 | 51223730 | 0.0529 | 0.6295 | 1.6497 | 1.4443 | 2 | 1 |
| WCM219 | 3 | 239459 | 197896683 | 0.0105 | −0.0712 | 1.0316 | 0.872 | 1 | 1 |
| WCM219 | 4 | 53422 | 190878508 | −0.0048 | −0.433 | 0.7605 | 0.721 | 1 | 1 |
| WCM219 | 5 | 90714 | 180687808 | −0.0018 | −0.3541 | 0.7824 | 0.7824 | 1 | 1 |
| WCM219 | 6 | 203583 | 170892883 | −0.0087 | −0.5386 | 0.7081 | 0.6688 | 1 | 1 |
| WCM219 | 7 | 193245 | 158937357 | 0.0054 | −0.1836 | 1.8149 | −0.0539 | 2 | 0 |
| WCM219 | 8 | 163470 | 146279424 | 0.0035 | −0.2253 | 0.8753 | 0.8356 | 1 | 1 |
| WCM219 | 9 | 116800 | 141107514 | 0.0329 | 0.3384 | 1.3657 | 1.163 | 1 | 1 |
| WCM219 | X | 2700084 | 154507335 | −0.1425 | NA | NA | NA | NA | NA |
| WCM223 | 1 | 14930 | 249213964 | 0.02 | 0.1589 | 1.1165 | 1.1165 | 1 | 1 |
| WCM223 | 10 | 225995 | 135439051 | 0.0223 | 0.1761 | 1.1298 | 1.1298 | 1 | 1 |
| WCM223 | 11 | 180170 | 63884918 | 0.0103 | 0.0834 | 1.0763 | 1.0427 | 1 | 1 |
| WCM223 | 11 | 63885182 | 65827316 | −0.177 | −4.7292 | 0.0377 | 0.0377 | 0 | 0 |
| WCM223 | 11 | 65828033 | 134605954 | 0.0064 | 0.0522 | 1.0368 | 1.0368 | 1 | 1 |
| WCM223 | 12 | 148485 | 133810788 | −0.0043 | −0.0365 | 0.975 | 0.975 | 1 | 1 |
| WCM223 | 13 | 19041937 | 115089411 | −0.0032 | −0.0272 | 0.9813 | 0.9813 | 1 | 1 |
| WCM223 | 14 | 19110297 | 107283269 | 0.0017 | 0.0144 | 1.01 | 1.01 | 1 | 1 |
| WCM223 | 15 | 20083896 | 102506269 | 0.0285 | 0.2221 | 1.1664 | 1.1664 | 1 | 1 |
| WCM223 | 16 | 74427 | 90168828 | −0.014 | −0.1213 | 0.9194 | 0.9194 | 1 | 1 |
| WCM223 | 17 | 131595 | 81052545 | 0.0059 | 0.0482 | 1.034 | 1.034 | 1 | 1 |
| WCM223 | 18 | 47610 | 78005146 | 0.0034 | 0.0283 | 1.0198 | 1.0198 | 1 | 1 |
| WCM223 | 19 | 104935 | 59093696 | −0.0465 | −0.4427 | 0.7357 | 0.7357 | 1 | 1 |
| WCM223 | 2 | 41670 | 243037103 | 0.0025 | 0.0205 | 1.0143 | 1.0143 | 1 | 1 |
| WCM223 | 20 | 68299 | 62934541 | 0.0118 | 0.0956 | 1.0685 | 1.0685 | 1 | 1 |
| WCM223 | 21 | 9590292 | 48084211 | 0.002 | 0.0165 | 1.0115 | 1.0115 | 1 | 1 |
| WCM223 | 22 | 16159079 | 51219089 | 0.0275 | 0.2144 | 1.1602 | 1.1602 | 1 | 1 |
| WCM223 | 3 | 239443 | 197896800 | 0.0021 | 0.0174 | 1.0121 | 1.0121 | 1 | 1 |
| WCM223 | 4 | 53375 | 190904482 | −0.0031 | −0.0259 | 0.9822 | 0.9822 | 1 | 1 |
| WCM223 | 5 | 90680 | 180687808 | 6.00E−04 | 0.0054 | 1.0037 | 1.0037 | 1 | 1 |
| WCM223 | 6 | 203515 | 170892869 | 0.0026 | 0.0215 | 1.015 | 1.015 | 1 | 1 |
| WCM223 | 7 | 193189 | 158937018 | −0.0069 | −0.0582 | 0.9605 | 0.9605 | 1 | 1 |
| WCM223 | 8 | 163461 | 146279489 | −0.0075 | −0.0639 | 0.9567 | 0.9567 | 1 | 1 |
| WCM223 | 9 | 116867 | 141107535 | 0.0148 | 0.1193 | 1.0862 | 1.0862 | 1 | 1 |
| WCM223 | X | 2700087 | 154493598 | −0.0839 | −0.9181 | NA | NA | NA | NA |
| WCM223_Z2_1 | 1 | 69594 | 17958926 | −0.0067 | −0.082 | 0.9576 | 0.932 | 1 | 1 |
| WCM223_Z2_1 | 1 | 17961381 | 32560384 | −0.1532 | −0.2487 | 0.9459 | 0.7374 | 1 | 1 |
| WCM223_Z2_1 | 1 | 32566212 | 249212550 | 0.0218 | −0.0498 | 1.0347 | 0.8974 | 1 | 1 |
| WCM223_Z2_1 | 10 | 225952 | 135440086 | −0.106 | −0.1948 | 1.0163 | 0.7311 | 1 | 1 |
| WCM223_Z2_1 | 11 | 193146 | 36657634 | 0.0482 | −0.0201 | 1.0794 | 0.893 | 1 | 1 |
| WCM223_Z2_1 | 11 | 36669589 | 49080617 | −0.8175 | −1.0426 | 0.871 | 0.0999 | 1 | 0 |
| WCM223_Z2_1 | 11 | 49089189 | 104034549 | 0.0349 | −0.035 | 0.987 | 0.965 | 1 | 1 |
| WCM223_Z2_1 | 11 | 104757520 | 106856850 | −0.8449 | −1.0771 | NA | NA | NA | NA |
| WCM223_Z2_1 | 11 | 107197755 | 134856574 | 0.039 | −0.0304 | 0.9869 | 0.9714 | 1 | 1 |
| WCM223_Z2_1 | 12 | 148811 | 124497463 | 0.0607 | −0.006 | 1.0877 | 0.9041 | 1 | 1 |
| WCM223_Z2_1 | 12 | 124796378 | 125396273 | −0.7154 | −0.9156 | NA | NA | NA | NA |
| WCM223_Z2_1 | 12 | 125397541 | 133810842 | 0.0382 | −0.0313 | 1.005 | 0.9521 | 1 | 1 |
| WCM223_Z2_1 | 13 | 19600515 | 40175311 | 0.0565 | −0.0106 | 1.0049 | 0.9804 | 1 | 1 |
| WCM223_Z2_1 | 13 | 40229957 | 58299417 | −0.7354 | −0.9403 | 1.0465 | −0.0043 | 1 | 0 |
| WCM223_Z2_1 | 13 | 60240829 | 72440384 | 0.1679 | 0.114 | NA | NA | NA | NA |
| WCM223_Z2_1 | 13 | 72440887 | 77663054 | −0.7502 | −0.9586 | 0.9964 | 0.0328 | 1 | 0 |
| WCM223_Z2_1 | 13 | 77664299 | 100295350 | 0.0768 | 0.0121 | 1.0265 | 0.9903 | 1 | 1 |
| WCM223_Z2_1 | 13 | 100511293 | 108518188 | −0.8426 | −1.0741 | 0.9771 | −0.0272 | 1 | 0 |
| WCM223_Z2_1 | 13 | 108518377 | 115089411 | 0.1001 | 0.0383 | 1.058 | 0.9958 | 1 | 1 |
| WCM223_Z2_1 | 14 | 20201899 | 107283184 | 0.0662 | 2.00E−04 | 1.0642 | 0.9361 | 1 | 1 |
| WCM223_Z2_1 | 15 | 20170028 | 102389740 | 0.0731 | 0.008 | 1.1004 | 0.9107 | 1 | 1 |
| WCM223_Z2_1 | 16 | 97462 | 49672433 | 0.0585 | −0.0084 | 1.0098 | 0.9785 | 1 | 1 |
| WCM223_Z2_1 | 16 | 49672761 | 84108282 | −0.7631 | −0.9747 | 1.0186 | −9.00E−04 | 1 | 0 |
| WCM223_Z2_1 | 16 | 84115406 | 90233626 | 0.1144 | 0.0543 | 1.1092 | 0.9675 | 1 | 1 |
| WCM223_Z2_1 | 17 | 131595 | 81083457 | 0.0574 | −0.0096 | 1.0056 | 0.9811 | 1 | 1 |
| WCM223_Z2_1 | 18 | 48235 | 78005160 | 0.0645 | −0.0017 | 1.01 | 0.9876 | 1 | 1 |
| WCM223_Z2_1 | 19 | 281507 | 59083929 | −0.1357 | −0.2287 | 0.9746 | 0.7323 | 1 | 1 |
| WCM223_Z2_1 | 2 | 41613 | 43015719 | 0.0676 | 0.0018 | 1.0597 | 0.9427 | 1 | 1 |
| WCM223_Z2_1 | 2 | 43019611 | 45235872 | −0.579 | −0.7492 | 1.0613 | 0.1286 | 1 | 0 |
| WCM223_Z2_1 | 2 | 45236148 | 114715029 | 0.079 | 0.0146 | 1.0249 | 0.9954 | 1 | 1 |
| WCM223_Z2_1 | 2 | 115200395 | 138425397 | −0.7513 | −0.96 | 1.0484 | −0.0203 | 1 | 0 |
| WCM223_Z2_1 | 2 | 138434157 | 148653997 | 0.0289 | −0.0418 | 1.0284 | 0.9145 | 1 | 1 |
| WCM223_Z2_1 | 2 | 148657382 | 162251649 | −0.695 | −0.8905 | 1.0798 | −9.00E−04 | 1 | 0 |
| WCM223_Z2_1 | 2 | 162265348 | 163302810 | 0.1683 | 0.1145 | NA | NA | NA | NA |
| WCM223_Z2_1 | 2 | 163360961 | 183291329 | −0.7523 | −0.9613 | 1.0316 | −0.0044 | 1 | 0 |
| WCM223_Z2_1 | 2 | 183387026 | 242946597 | 0.0664 | 4.00E−04 | 1.0565 | 0.9441 | 1 | 1 |
| WCM223_Z2_1 | 20 | 68373 | 62904835 | 0.0657 | −4.00E−04 | 1.0941 | 0.9054 | 1 | 1 |
| WCM223_Z2_1 | 21 | 9826235 | 48084282 | 0.0691 | 0.0035 | 1.0159 | 0.9889 | 1 | 1 |
| WCM223_Z2_1 | 22 | 16287443 | 51215137 | 0.0427 | −0.0262 | 0.9974 | 0.9665 | 1 | 1 |
| WCM223_Z2_1 | 3 | 239459 | 75832483 | 0.0643 | −0.0019 | 1.0548 | 0.9426 | 1 | 1 |
| WCM223_Z2_1 | 3 | 75986643 | 89391019 | −0.7013 | −0.8982 | NA | NA | NA | NA |
| WCM223_Z2_1 | 3 | 89391220 | 98309560 | 0.0629 | −0.0035 | 1.0368 | 0.9583 | 1 | 1 |
| WCM223_Z2_1 | 3 | 98309871 | 99683234 | −0.6816 | −0.874 | NA | NA | NA | NA |
| WCM223_Z2_1 | 3 | 99860401 | 118649049 | 0.082 | 0.018 | 1.0262 | 0.9989 | 1 | 1 |
| WCM223_Z2_1 | 3 | 118753396 | 120347345 | −0.7028 | −0.9001 | 1.0753 | −0.0036 | 1 | 0 |
| WCM223_Z2_1 | 3 | 120352070 | 197896714 | 0.0482 | −0.02 | 1.0007 | 0.9718 | 1 | 1 |
| WCM223_Z2_1 | 4 | 59327 | 190426502 | 0.0698 | 0.0043 | 1.0154 | 0.9906 | 1 | 1 |
| WCM223_Z2_1 | 5 | 140632 | 71495575 | 0.0761 | 0.0113 | 1.0215 | 0.9943 | 1 | 1 |
| WCM223_Z2_1 | 5 | 71496025 | 77524025 | −0.7166 | −0.917 | 1.0685 | −0.0093 | 1 | 0 |
| WCM223_Z2_1 | 5 | 77536725 | 96315010 | 0.0918 | 0.029 | 1.0592 | 0.9814 | 1 | 1 |
| WCM223_Z2_1 | 5 | 96315192 | 101816132 | −0.5911 | −0.7638 | NA | NA | NA | NA |
| WCM223_Z2_1 | 5 | 101834232 | 102611706 | −2.3815 | −3.6014 | NA | NA | NA | NA |
| WCM223_Z2_1 | 5 | 102611934 | 111312435 | −0.8076 | −1.0302 | NA | NA | NA | NA |
| WCM223_Z2_1 | 5 | 111479564 | 112178437 | −2.4942 | −3.9015 | NA | NA | NA | NA |
| WCM223_Z2_1 | 5 | 112178759 | 112927754 | −0.7545 | −0.9639 | 1.0274 | −0.0021 | 1 | 0 |
| WCM223_Z2_1 | 5 | 113698579 | 124080542 | 0.1064 | 0.0453 | 1.0654 | 0.9984 | 1 | 1 |
| WCM223_Z2_1 | 5 | 125696083 | 126993282 | −0.8064 | −1.0287 | NA | NA | NA | NA |
| WCM223_Z2_1 | 5 | 127419805 | 151784397 | 0.0603 | −0.0064 | 1.0247 | 0.9665 | 1 | 1 |
| WCM223_Z2_1 | 5 | 151784521 | 156887344 | −0.807 | −1.0295 | 0.9985 | −0.0187 | 1 | 0 |
| WCM223_Z2_1 | 5 | 156887583 | 167489211 | 0.0651 | −0.001 | 1.0756 | 0.9229 | 1 | 1 |
| WCM223_Z2_1 | 5 | 167517764 | 169186755 | −0.8338 | −1.0631 | 0.9941 | −0.0369 | 1 | 0 |
| WCM223_Z2_1 | 5 | 169188534 | 180687804 | 0.0593 | −0.0075 | 1.0216 | 0.9679 | 1 | 1 |
| WCM223_Z2_1 | 6 | 203545 | 70733547 | 0.0262 | −0.0447 | 1.0318 | 0.9072 | 1 | 1 |
| WCM223_Z2_1 | 6 | 70744183 | 100838345 | −0.7825 | −0.9988 | 1.0215 | −0.0206 | 1 | 0 |
| WCM223_Z2_1 | 6 | 100838569 | 170893450 | 0.0319 | −0.0384 | 0.9924 | 0.9551 | 1 | 1 |
| WCM223_Z2_1 | 7 | 193451 | 158937416 | 0.0558 | −0.0115 | 1.0836 | 0.9006 | 1 | 1 |
| WCM223_Z2_1 | 8 | 190900 | 146279527 | 0.0619 | −0.0046 | 1.008 | 0.9856 | 1 | 1 |
| WCM223_Z2_1 | 9 | 17066 | 141111414 | 0.0508 | −0.017 | 0.9972 | 0.9794 | 1 | 1 |
| WCM223_Z2_1 | X | 2700146 | 154509348 | −0.0286 | −0.1068 | NA | NA | NA | NA |
| WCM223_Z4_1 | 1 | 69569 | 46685865 | 0.0408 | 0.036 | 1.0339 | 1.0165 | 1 | 1 |
| WCM223_Z4_1 | 1 | 46714242 | 49711536 | 0.4907 | 0.5097 | 1.9459 | 0.9016 | 2 | 1 |
| WCM223_Z4_1 | 1 | 50162959 | 174769612 | −0.0572 | −0.0684 | 0.9537 | 0.9537 | 1 | 1 |
| WCM223_Z4_1 | 1 | 174781061 | 249212557 | 0.058 | 0.0542 | 1.0383 | 1.0383 | 1 | 1 |
| WCM223_Z4_1 | 10 | 225960 | 135440108 | 0.0502 | 0.046 | 1.0324 | 1.0324 | 1 | 1 |
| WCM223_Z4_1 | 11 | 193146 | 36657651 | 0.0557 | 0.0518 | 1.0464 | 1.0266 | 1 | 1 |
| WCM223_Z4_1 | 11 | 36669604 | 48511269 | −0.8631 | −0.9484 | 0.9775 | 0.059 | 1 | 0 |
| WCM223_Z4_1 | 11 | 48997230 | 104034537 | 0.0496 | 0.0453 | 1.0319 | 1.0319 | 1 | 1 |
| WCM223_Z4_1 | 11 | 104757470 | 106856849 | −0.9242 | −1.0173 | 0.9855 | 0.0026 | 1 | 0 |
| WCM223_Z4_1 | 11 | 107197725 | 134856597 | 0.029 | 0.0234 | 1.0163 | 1.0163 | 1 | 1 |
| WCM223_Z4_1 | 12 | 148644 | 124497381 | 0.0566 | 0.0527 | 1.0416 | 1.0328 | 1 | 1 |
| WCM223_Z4_1 | 12 | 124497466 | 125396262 | −0.7638 | −0.8373 | NA | NA | NA | NA |
| WCM223_Z4_1 | 12 | 125397541 | 133810788 | 0.0424 | 0.0377 | 1.0461 | 1.0068 | 1 | 1 |
| WCM223_Z4_1 | 13 | 19600371 | 40175311 | 0.0686 | 0.0654 | 1.0586 | 1.0342 | 1 | 1 |
| WCM223_Z4_1 | 13 | 40229957 | 58299425 | −0.8994 | −0.9893 | 1.0111 | −0.0037 | 1 | 0 |
| WCM223_Z4_1 | 13 | 60240829 | 72440887 | −0.1664 | −0.1852 | NA | NA | NA | NA |
| WCM223_Z4_1 | 13 | 73284470 | 77661658 | −0.942 | −1.0373 | 1.0124 | −0.0379 | 1 | 0 |
| WCM223_Z4_1 | 13 | 77663003 | 100295349 | −0.0533 | −0.0642 | 0.9697 | 0.9432 | 1 | 1 |
| WCM223_Z4_1 | 13 | 100511205 | 108183525 | −0.9593 | −1.0569 | 0.9407 | 0.0206 | 1 | 0 |
| WCM223_Z4_1 | 13 | 108518049 | 115089411 | −0.0116 | −0.0197 | 0.998 | 0.9749 | 1 | 1 |
| WCM223_Z4_1 | 14 | 19377733 | 107283183 | −0.04 | −0.0501 | 0.972 | 0.9597 | 1 | 1 |
| WCM223_Z4_1 | 15 | 20169982 | 102389740 | −0.0439 | −0.0542 | 0.9734 | 0.9529 | 1 | 1 |
| WCM223_Z4_1 | 16 | 97439 | 49672681 | −0.036 | −0.0458 | 0.9791 | 0.9584 | 1 | 1 |
| WCM223_Z4_1 | 16 | 49764837 | 84108301 | −0.92 | −1.0125 | 0.9931 | −0.0017 | 1 | 0 |
| WCM223_Z4_1 | 16 | 84115502 | 90233642 | −0.0184 | −0.027 | 0.995 | 0.9679 | 1 | 1 |
| WCM223_Z4_1 | 17 | 131595 | 81083561 | −0.0149 | −0.0233 | 0.9882 | 0.9798 | 1 | 1 |
| WCM223_Z4_1 | 18 | 48269 | 78005215 | −0.0627 | −0.0743 | 0.962 | 0.9376 | 1 | 1 |
| WCM223_Z4_1 | 19 | 281445 | 59083970 | −0.1943 | −0.2151 | 0.9919 | 0.731 | 1 | 1 |
| WCM223_Z4_1 | 2 | 41609 | 114492232 | −0.0478 | −0.0583 | 0.9655 | 0.9553 | 1 | 1 |
| WCM223_Z4_1 | 2 | 114493367 | 138727740 | −0.9046 | −0.9951 | 1.0095 | −0.0061 | 1 | 0 |
| WCM223_Z4_1 | 2 | 138738916 | 147346315 | −0.1442 | −0.1614 | 0.9047 | 0.8836 | 1 | 1 |
| WCM223_Z4_1 | 2 | 147596973 | 162251613 | −0.958 | −1.0555 | 0.9792 | −0.017 | 1 | 0 |
| WCM223_Z4_1 | 2 | 162265398 | 163374493 | −0.0113 | −0.0195 | NA | NA | NA | NA |
| WCM223_Z4_1 | 2 | 163393463 | 183291329 | −0.9607 | −1.0585 | 0.9626 | −0.0024 | 1 | 0 |
| WCM223_Z4_1 | 2 | 183387026 | 242946597 | −0.0524 | −0.0632 | 0.9622 | 0.952 | 1 | 1 |
| WCM223_Z4_1 | 20 | 68397 | 62904890 | −0.0143 | −0.0227 | 0.9844 | 0.9844 | 1 | 1 |
| WCM223_Z4_1 | 21 | 9826235 | 35987113 | −0.0729 | −0.0851 | 0.9759 | 0.9095 | 1 | 1 |
| WCM223_Z4_1 | 21 | 36041973 | 42830468 | −0.9395 | −1.0346 | 0.964 | 0.0123 | 1 | 0 |
| WCM223_Z4_1 | 21 | 42838075 | 48084282 | −0.0203 | −0.0291 | 0.9978 | 0.9623 | 1 | 1 |
| WCM223_Z4_1 | 22 | 16287481 | 51215134 | −0.0023 | −0.0099 | 0.9932 | 0.9932 | 1 | 1 |
| WCM223_Z4_1 | 3 | 239472 | 75986730 | −0.0338 | −0.0434 | 0.9745 | 0.9662 | 1 | 1 |
| WCM223_Z4_1 | 3 | 77147269 | 89259670 | −0.9195 | −1.0119 | NA | NA | NA | NA |
| WCM223_Z4_1 | 3 | 89390222 | 98312348 | −0.0979 | −0.1118 | 0.962 | 0.8888 | 1 | 1 |
| WCM223_Z4_1 | 3 | 98451717 | 99683234 | −0.9506 | −1.047 | NA | NA | NA | NA |
| WCM223_Z4_1 | 3 | 99860401 | 118753415 | −0.0602 | −0.0716 | 0.9739 | 0.9292 | 1 | 1 |
| WCM223_Z4_1 | 3 | 118865097 | 120347292 | −0.8865 | −0.9747 | 0.9985 | 0.0192 | 1 | 0 |
| WCM223_Z4_1 | 3 | 120352064 | 197896720 | −0.0411 | −0.0513 | 0.9744 | 0.9558 | 1 | 1 |
| WCM223_Z4_1 | 4 | 59334 | 190426471 | 0.0382 | 0.0332 | 1.0309 | 1.0156 | 1 | 1 |
| WCM223_Z4_1 | 5 | 140666 | 71499532 | 0.0461 | 0.0415 | 1.0401 | 1.0183 | 1 | 1 |
| WCM223_Z4_1 | 5 | 71500934 | 77524037 | −0.8401 | −0.9226 | 1.0764 | −0.0212 | 1 | 0 |
| WCM223_Z4_1 | 5 | 77536725 | 96253296 | 0.0191 | 0.0129 | 1.0347 | 0.9832 | 1 | 1 |
| WCM223_Z4_1 | 5 | 96315004 | 101834442 | −0.7876 | −0.8639 | NA | NA | NA | NA |
| WCM223_Z4_1 | 5 | 102237108 | 102612076 | −3.1008 | −4.0691 | NA | NA | NA | NA |
| WCM223_Z4_1 | 5 | 102612215 | 111311009 | −0.8139 | −0.8933 | NA | NA | NA | NA |
| WCM223_Z4_1 | 5 | 111312435 | 112178381 | −3.0827 | −4.0319 | NA | NA | NA | NA |
| WCM223_Z4_1 | 5 | 112178770 | 112927786 | −0.8318 | −0.9133 | 1.0461 | 0.0159 | 1 | 0 |
| WCM223_Z4_1 | 5 | 113698705 | 124080262 | 0.1019 | 0.1008 | 1.1121 | 1.0326 | 1 | 1 |
| WCM223_Z4_1 | 5 | 124080309 | 126993282 | −0.8141 | −0.8935 | NA | NA | NA | NA |
| WCM223_Z4_1 | 5 | 127419802 | 151784655 | 0.0472 | 0.0427 | 1.0355 | 1.0246 | 1 | 1 |
| WCM223_Z4_1 | 5 | 152869386 | 156887202 | −0.8435 | −0.9264 | 1.079 | −0.0267 | 1 | 0 |
| WCM223_Z4_1 | 5 | 156887583 | 167489255 | 0.0391 | 0.0341 | 1.0587 | 0.9891 | 1 | 1 |
| WCM223_Z4_1 | 5 | 167517646 | 169188527 | −0.8062 | −0.8847 | 1.1234 | −0.0402 | 1 | 0 |
| WCM223_Z4_1 | 5 | 169230068 | 180687808 | 0.0332 | 0.0279 | 1.0293 | 1.0098 | 1 | 1 |
| WCM223_Z4_1 | 6 | 203542 | 70733547 | 0.0539 | 0.0498 | 1.0352 | 1.0352 | 1 | 1 |
| WCM223_Z4_1 | 6 | 70744192 | 100403928 | −0.8417 | −0.9244 | 1.0574 | −0.0036 | 1 | 0 |
| WCM223_Z4_1 | 6 | 100838312 | 170893411 | 0.0462 | 0.0416 | 1.0325 | 1.026 | 1 | 1 |
| WCM223_Z4_1 | 7 | 193245 | 158937416 | 0.0477 | 0.0433 | 1.0359 | 1.025 | 1 | 1 |
| WCM223_Z4_1 | 8 | 190906 | 21608453 | 0.0752 | 0.0725 | 1.0694 | 1.0337 | 1 | 1 |
| WCM223_Z4_1 | 8 | 21766846 | 30335312 | −0.8669 | −0.9526 | 1.0339 | −6.00E−04 | 1 | 0 |
| WCM223_Z4_1 | 8 | 30336893 | 146279498 | 0.0716 | 0.0686 | 1.0576 | 1.0398 | 1 | 1 |
| WCM223_Z4_1 | 9 | 17055 | 141111414 | 0.0502 | 0.0459 | 1.0323 | 1.0323 | 1 | 1 |
| WCM223_Z4_1 | X | 2700139 | 154509348 | 0.0209 | 0.0148 | NA | NA | NA | NA |
| WCM243 | 1 | 14930 | 6221386 | 0.0113 | 0.2532 | 1.1918 | 1.1918 | 1 | 1 |
| WCM243 | 1 | 6226299 | 109897022 | −0.1503 | −0.6402 | 0.6416 | 0.6416 | 1 | 1 |
| WCM243 | 1 | 109897983 | 214781971 | 0.1493 | 0.7763 | 1.7127 | 1.7127 | 2 | 2 |
| WCM243 | 1 | 214787266 | 235902124 | −0.0604 | −0.0892 | 1.2266 | 0.6535 | 1 | 1 |
| WCM243 | 1 | 235904395 | 249214018 | 0.1588 | 0.808 | 1.7508 | 1.7508 | 2 | 2 |
| WCM243 | 10 | 226083 | 135438994 | −0.037 | 0.0296 | 1.0207 | 1.0207 | 1 | 1 |
| WCM243 | 11 | 193096 | 9769562 | 0.012 | 0.2561 | 1.1942 | 1.1942 | 1 | 1 |
| WCM243 | 11 | 9770622 | 93803559 | 0.0903 | 0.5696 | 1.4842 | 1.4842 | 1 | 1 |
| WCM243 | 11 | 93806210 | 134320516 | −0.0792 | −0.191 | 0.876 | 0.876 | 1 | 1 |
| WCM243 | 12 | 148452 | 69145785 | −0.1835 | −0.8994 | 0.5361 | 0.5361 | 1 | 1 |
| WCM243 | 12 | 69152951 | 133345574 | 0.0094 | 0.2449 | 1.185 | 1.185 | 1 | 1 |
| WCM243 | 12 | 133347659 | 133810794 | −0.2109 | −1.1501 | 0.4506 | 0.4506 | 0 | 0 |
| WCM243 | 13 | 19041992 | 115089411 | 0.0952 | 0.5875 | 1.5027 | 1.5027 | 2 | 2 |
| WCM243 | 14 | 19110343 | 107283169 | 0.0213 | 0.296 | 1.2277 | 1.2277 | 1 | 1 |
| WCM243 | 15 | 20083896 | 63986772 | 0.1258 | 0.6965 | 1.6205 | 1.6205 | 2 | 2 |
| WCM243 | 15 | 63987016 | 102304817 | −0.1427 | −0.5865 | 0.666 | 0.666 | 1 | 1 |
| WCM243 | 15 | 102311872 | 102506258 | 0.1378 | 0.7378 | NA | NA | NA | NA |
| WCM243 | 16 | 74369 | 48218471 | −0.053 | −0.0507 | 0.9655 | 0.9655 | 1 | 1 |
| WCM243 | 16 | 48221117 | 81185425 | −0.1975 | −1.0222 | 0.5373 | 0.4474 | 1 | 0 |
| WCM243 | 16 | 81187685 | 90260461 | −0.161 | −0.7192 | 0.6074 | 0.6074 | 1 | 1 |
| WCM243 | 17 | 131595 | 8508157 | −0.0552 | −0.0622 | 1.9741 | −0.0586 | 2 | 0 |
| WCM243 | 17 | 8526369 | 39791573 | 0.04 | 0.3741 | 2.5406 | 0.0515 | 3 | 0 |
| WCM243 | 17 | 39845329 | 81187853 | −0.1442 | −0.5968 | 0.6612 | 0.6612 | 1 | 1 |
| WCM243 | 18 | 47692 | 78005146 | 0.0282 | 0.3253 | 1.2529 | 1.2529 | 1 | 1 |
| WCM243 | 19 | 105080 | 1277169 | −0.0567 | −0.0698 | 1.2797 | 0.6259 | 1 | 1 |
| WCM243 | 19 | 1278977 | 31767287 | 0.0685 | 0.4871 | 1.4017 | 1.4017 | 1 | 1 |
| WCM243 | 19 | 31767676 | 43023375 | −0.1845 | −0.9078 | 0.533 | 0.533 | 1 | 1 |
| WCM243 | 19 | 43025541 | 43920730 | 0.4938 | 1.702 | 3.7758 | 2.7313 | 4 | 3 |
| WCM243 | 19 | 43922159 | 59093645 | −0.1793 | −0.8645 | 0.5492 | 0.5492 | 1 | 1 |
| WCM243 | 2 | 41647 | 243037116 | −0.1635 | −0.7389 | 0.5992 | 0.5992 | 1 | 1 |
| WCM243 | 20 | 68363 | 42574625 | −0.0024 | 0.1925 | 1.1427 | 1.1427 | 1 | 1 |
| WCM243 | 20 | 42597482 | 62934572 | 0.3195 | 1.28 | 2.4284 | 2.4284 | 2 | 2 |
| WCM243 | 21 | 9590301 | 48084253 | −0.0988 | −0.3037 | 0.8102 | 0.8102 | 1 | 1 |
| WCM243 | 22 | 16159014 | 51216306 | −0.0452 | −0.0112 | 0.9922 | 0.9922 | 1 | 1 |
| WCM243 | 3 | 239531 | 197894690 | −0.0526 | −0.0488 | 0.9667 | 0.9667 | 1 | 1 |
| WCM243 | 4 | 53364 | 190873327 | −0.0929 | −0.2686 | 0.8301 | 0.8301 | 1 | 1 |
| WCM243 | 5 | 90647 | 52285275 | −0.0219 | 0.1023 | 1.0735 | 1.0735 | 1 | 1 |
| WCM243 | 5 | 52322635 | 148377838 | −0.19 | −0.9557 | 0.5156 | 0.5156 | 1 | 1 |
| WCM243 | 5 | 148381846 | 180687830 | 0.0611 | 0.4584 | 1.374 | 1.374 | 1 | 1 |
| WCM243 | 6 | 203475 | 170892848 | −0.0671 | −0.1246 | 0.9172 | 0.9172 | 1 | 1 |
| WCM243 | 7 | 193253 | 158937018 | 0.1378 | 0.7379 | 1.7131 | 1.6224 | 2 | 2 |
| WCM243 | 8 | 166429 | 146279383 | 0.0278 | 0.3234 | 1.2513 | 1.2513 | 1 | 1 |
| WCM243 | 9 | 116867 | 105768000 | −0.1719 | −0.8043 | 0.5727 | 0.5727 | 1 | 1 |
| WCM243 | 9 | 106857798 | 141013089 | −0.0243 | 0.0909 | 1.5412 | 0.5889 | 2 | 1 |
| WCM243 | 9 | 141014652 | 141107513 | −0.0408 | 0.0112 | NA | NA | NA | NA |
| WCM243 | X | 2700151 | 154493340 | −0.2346 | −1.4029 | NA | NA | NA | NA |
| WCM243_X1_1 | 1 | 69603 | 878432 | 0.0032 | 0.6782 | NA | NA | NA | NA |
| WCM243_X1_1 | 1 | 878700 | 15863120 | −0.0419 | 0.6161 | 2.0352 | 1.0303 | 2 | 1 |
| WCM243_X1_1 | 1 | 15870980 | 50460823 | −0.4439 | 0.0245 | 1.0589 | 0.9754 | 1 | 1 |
| WCM243_X1_1 | 1 | 50460995 | 53925184 | −0.9393 | −0.8603 | 0.9275 | 0.1741 | 1 | 0 |
| WCM243_X1_1 | 1 | 53925585 | 109940443 | −0.4287 | 0.0485 | 1.1639 | 0.9045 | 1 | 1 |
| WCM243_X1_1 | 1 | 109944699 | 120611999 | 0.2419 | 0.9954 | 3.0286 | 0.9587 | 3 | 1 |
| WCM243_X1_1 | 1 | 143767833 | 214822081 | 0.4998 | 1.3212 | 2.9525 | 2.0451 | 3 | 2 |
| WCM243_X1_1 | 1 | 214825220 | 236175291 | −0.0485 | 0.607 | 3.0405 | 0.0057 | 3 | 0 |
| WCM243_X1_1 | 1 | 236176739 | 249212366 | 0.5036 | 1.3259 | 2.9463 | 2.0676 | 3 | 2 |
| WCM243_X1_1 | 10 | 225980 | 135440086 | −0.0515 | 0.6029 | 1.9926 | 1.0449 | 2 | 1 |
| WCM243_X1_1 | 11 | 193146 | 34511577 | 0.2751 | 1.0382 | 2.0854 | 2.022 | 2 | 2 |
| WCM243_X1_1 | 11 | 34515066 | 36596676 | 0.4591 | 1.2708 | 3.0791 | 1.7469 | 3 | 2 |
| WCM243_X1_1 | 11 | 36597061 | 48285558 | 0.5189 | 1.3448 | 3.9597 | 1.1202 | 4 | 1 |
| WCM243_X1_1 | 11 | 48285705 | 77594926 | 0.491 | 1.3104 | 3.0262 | 1.9342 | 3 | 2 |
| WCM243_X1_1 | 11 | 77595863 | 93400796 | 0.308 | 1.0805 | 2.212 | 2.0175 | 2 | 2 |
| WCM243_X1_1 | 11 | 93402856 | 134856523 | −0.0573 | 0.5948 | 1.9906 | 1.0299 | 2 | 1 |
| WCM243_X1_1 | 12 | 148673 | 69265683 | −0.4329 | 0.0419 | 1.0489 | 1.0101 | 1 | 1 |
| WCM243_X1_1 | 12 | 69279662 | 112893752 | −0.0542 | 0.599 | 2.0885 | 0.9409 | 2 | 1 |
| WCM243_X1_1 | 12 | 112910815 | 133810926 | 0.2475 | 1.0026 | 2.0037 | 2.0037 | 2 | 2 |
| WCM243_X1_1 | 13 | 19600365 | 29291983 | 0.5171 | 1.3427 | 4.1198 | 0.9526 | 4 | 1 |
| WCM243_X1_1 | 13 | 29598959 | 54886136 | 0.251 | 1.0072 | 3.0021 | 1.0178 | 3 | 1 |
| WCM243_X1_1 | 13 | 55748673 | 97639984 | 0.487 | 1.3054 | 4.0775 | 0.8657 | 4 | 1 |
| WCM243_X1_1 | 13 | 97928639 | 115089411 | 0.1941 | 0.9333 | 2.7786 | 1.0407 | 3 | 1 |
| WCM243_X1_1 | 14 | 19377727 | 20770020 | −0.03 | 0.6326 | 2.198 | 0.9026 | 2 | 1 |
| WCM243_X1_1 | 14 | 20774081 | 21359923 | 0.2851 | 1.0511 | 3.0614 | 1.0828 | 3 | 1 |
| WCM243_X1_1 | 14 | 21360144 | 38218418 | −0.04 | 0.6188 | 2.0259 | 1.0452 | 2 | 1 |
| WCM243_X1_1 | 14 | 38220376 | 107283085 | 0.2486 | 1.004 | 2.0238 | 1.9874 | 2 | 2 |
| WCM243_X1_1 | 15 | 20170011 | 22382897 | −0.0025 | 0.6704 | 2.5959 | 0.5871 | 3 | 1 |
| WCM243_X1_1 | 15 | 22383189 | 42680058 | 0.2343 | 0.9856 | 2.9301 | 1.0302 | 3 | 1 |
| WCM243_X1_1 | 15 | 42681250 | 43132576 | −0.4096 | 0.0782 | NA | NA | NA | NA |
| WCM243_X1_1 | 15 | 43164810 | 43483717 | 0.31 | 1.0829 | NA | NA | NA | NA |
| WCM243_X1_1 | 15 | 43484981 | 52622650 | 0.7091 | 1.5756 | 5.0143 | 0.9469 | 5 | 1 |
| WCM243_X1_1 | 15 | 52628719 | 63970465 | 0.2633 | 1.023 | 2.9359 | 1.1285 | 3 | 1 |
| WCM243_X1_1 | 15 | 63972297 | 102514214 | −0.4405 | 0.03 | 1.132 | 0.91 | 1 | 1 |
| WCM243_X1_1 | 16 | 67604 | 46508225 | −0.0424 | 0.6154 | 2.0194 | 1.0446 | 2 | 1 |
| WCM243_X1_1 | 16 | 46509904 | 48212572 | 0.2639 | 1.0238 | NA | NA | NA | NA |
| WCM243_X1_1 | 16 | 48218460 | 90233603 | −0.4357 | 0.0375 | 2.03 | 0.0226 | 2 | 0 |
| WCM243_X1_1 | 17 | 131595 | 4356483 | 0.2092 | 0.9529 | 3.8962 | −0.0247 | 4 | 0 |
| WCM243_X1_1 | 17 | 4381921 | 4925943 | −0.0436 | 0.6137 | 3.0866 | −0.0262 | 3 | 0 |
| WCM243_X1_1 | 17 | 4926882 | 7216124 | 0.1879 | 0.9251 | 3.8182 | −0.0205 | 4 | 0 |
| WCM243_X1_1 | 17 | 7216586 | 7592579 | −1.0079 | −1.0057 | 1.0299 | −0.0338 | 1 | 0 |
| WCM243_X1_1 | 17 | 7592957 | 17696270 | −0.0515 | 0.6028 | 3.0223 | 0.015 | 3 | 0 |
| WCM243_X1_1 | 17 | 17696750 | 21191389 | 0.2324 | 0.9831 | 3.9639 | −0.0104 | 4 | 0 |
| WCM243_X1_1 | 17 | 21194820 | 22023445 | 0.1154 | 0.8294 | 2.0985 | 1.4554 | 2 | 1 |
| WCM243_X1_1 | 17 | 25621441 | 40265724 | 0.2357 | 0.9874 | 3.9255 | 0.0398 | 4 | 0 |
| WCM243_X1_1 | 17 | 40266254 | 56060735 | −0.0524 | 0.6016 | 3.0265 | 0.0083 | 3 | 0 |
| WCM243_X1_1 | 17 | 56082752 | 81083567 | −0.4542 | 0.0082 | 1.9806 | 0.0308 | 2 | 0 |
| WCM243_X1_1 | 18 | 48190 | 78005164 | 0.2182 | 0.9647 | 1.9719 | 1.9313 | 2 | 2 |
| WCM243_X1_1 | 19 | 281503 | 32083868 | 0.2276 | 0.9769 | 2.8832 | 1.0534 | 3 | 1 |
| WCM243_X1_1 | 19 | 32843799 | 59083923 | −0.462 | −0.0041 | 1.1318 | 0.8625 | 1 | 1 |
| WCM243_X1_1 | 2 | 41612 | 44079913 | −0.4181 | 0.0651 | 1.0723 | 1.02 | 1 | 1 |
| WCM243_X1_1 | 2 | 44099168 | 47085320 | −0.8972 | −0.7746 | NA | NA | NA | NA |
| WCM243_X1_1 | 2 | 47132711 | 242946607 | −0.4331 | 0.0415 | 1.0664 | 0.992 | 1 | 1 |
| WCM243_X1_1 | 20 | 68397 | 26188912 | −0.0504 | 0.6043 | 2.0303 | 1.0102 | 2 | 1 |
| WCM243_X1_1 | 20 | 29632632 | 39726959 | 0.2572 | 1.0152 | 2.1568 | 1.8855 | 2 | 2 |
| WCM243_X1_1 | 20 | 39728812 | 42602040 | −0.0191 | 0.6476 | NA | NA | NA | NA |
| WCM243_X1_1 | 20 | 42635393 | 62904861 | 0.8828 | 1.7812 | 4.9462 | 1.9279 | 5 | 2 |
| WCM243_X1_1 | 21 | 9826225 | 39822803 | −0.058 | 0.5938 | 1.9118 | 1.1067 | 2 | 1 |
| WCM243_X1_1 | 21 | 39947608 | 43133145 | −0.4193 | 0.0632 | 2.073 | 0.0166 | 2 | 0 |
| WCM243_X1_1 | 21 | 43133829 | 44195395 | 0.0155 | 0.6949 | 2.0931 | 1.1445 | 2 | 1 |
| WCM243_X1_1 | 21 | 44270183 | 48056892 | −0.4281 | 0.0495 | 2.0601 | 0.0097 | 2 | 0 |
| WCM243_X1_1 | 21 | 48063531 | 48084281 | −0.1265 | 0.4977 | NA | NA | NA | NA |
| WCM243_X1_1 | 22 | 16287478 | 21576160 | 0.2498 | 1.0057 | 2.0935 | 1.9223 | 2 | 2 |
| WCM243_X1_1 | 22 | 21799370 | 29759016 | −0.0783 | 0.5656 | 2.0795 | 0.8805 | 2 | 1 |
| WCM243_X1_1 | 22 | 29815167 | 31795693 | −0.4727 | −0.0211 | 1.9292 | 0.0418 | 2 | 0 |
| WCM243_X1_1 | 22 | 31796610 | 51219138 | −0.0739 | 0.5716 | 2.0212 | 0.9512 | 2 | 1 |
| WCM243_X1_1 | 3 | 239524 | 68055826 | −0.0562 | 0.5963 | 2.0052 | 1.0186 | 2 | 1 |
| WCM243_X1_1 | 3 | 68466539 | 74351914 | 0.4053 | 1.2037 | NA | NA | NA | NA |
| WCM243_X1_1 | 3 | 74384005 | 97311581 | −0.0816 | 0.5609 | NA | NA | NA | NA |
| WCM243_X1_1 | 3 | 97329692 | 116163815 | 0.2557 | 1.0132 | 2.0732 | 1.9637 | 2 | 2 |
| WCM243_X1_1 | 3 | 116569172 | 120976170 | −0.0342 | 0.6268 | 2.1814 | 0.9068 | 2 | 1 |
| WCM243_X1_1 | 3 | 120977904 | 125199137 | −0.4382 | 0.0336 | 1.974 | 0.0732 | 2 | 0 |
| WCM243_X1_1 | 3 | 125216198 | 197894713 | −0.0477 | 0.6081 | 2.0015 | 1.047 | 2 | 1 |
| WCM243_X1_1 | 4 | 59405 | 40440867 | −0.4302 | 0.0462 | 1.0633 | 1.0018 | 1 | 1 |
| WCM243_X1_1 | 4 | 40504083 | 49063839 | −0.0353 | 0.6253 | 2.1623 | 0.9227 | 2 | 1 |
| WCM243_X1_1 | 4 | 52709800 | 54880275 | −0.9531 | −0.8888 | NA | NA | NA | NA |
| WCM243_X1_1 | 4 | 54915170 | 57976487 | −0.4383 | 0.0334 | 1.1201 | 0.9268 | 1 | 1 |
| WCM243_X1_1 | 4 | 61788391 | 69434201 | −0.9229 | −0.8265 | NA | NA | NA | NA |
| WCM243_X1_1 | 4 | 69696596 | 125591686 | −0.0395 | 0.6195 | 2.0312 | 1.0415 | 2 | 1 |
| WCM243_X1_1 | 4 | 125591799 | 138453409 | −0.4471 | 0.0195 | NA | NA | NA | NA |
| WCM243_X1_1 | 4 | 139100419 | 152498625 | −0.0398 | 0.6191 | 3.1325 | −0.0608 | 3 | 0 |
| WCM243_X1_1 | 4 | 152499055 | 156643299 | −0.3277 | 0.2038 | 2.2352 | 0.0683 | 2 | 0 |
| WCM243_X1_1 | 4 | 156651366 | 156764911 | −1.7778 | −4.3452 | NA | NA | NA | NA |
| WCM243_X1_1 | 4 | 157555881 | 159590915 | −0.3774 | 0.1281 | NA | NA | NA | NA |
| WCM243_X1_1 | 4 | 159592912 | 178257420 | −0.0572 | 0.595 | 2.874 | 0.1469 | 3 | 0 |
| WCM243_X1_1 | 4 | 178260970 | 178911657 | 0.0321 | 0.7175 | NA | NA | NA | NA |
| WCM243_X1_1 | 4 | 183090470 | 186064618 | −0.7729 | −0.5354 | 1.3797 | 2.00E−04 | 1 | 0 |
| WCM243_X1_1 | 4 | 186066098 | 190426570 | −0.0689 | 0.5787 | 1.9201 | 1.0669 | 2 | 1 |
| WCM243_X1_1 | 5 | 140532 | 52145311 | −0.0417 | 0.6164 | 1.9338 | 1.1323 | 2 | 1 |
| WCM243_X1_1 | 5 | 52157358 | 78938702 | −0.4366 | 0.036 | 1.0446 | 1.0059 | 1 | 1 |
| WCM243_X1_1 | 5 | 78941013 | 79768623 | −0.9167 | −0.8138 | NA | NA | NA | NA |
| WCM243_X1_1 | 5 | 79770520 | 83680163 | −0.4066 | 0.083 | 1.1777 | 0.9406 | 1 | 1 |
| WCM243_X1_1 | 5 | 85578660 | 112927811 | −0.955 | −0.8928 | 1.0531 | 0.024 | 1 | 0 |
| WCM243_X1_1 | 5 | 112929081 | 148045072 | −0.4342 | 0.0399 | 1.0506 | 1.0054 | 1 | 1 |
| WCM243_X1_1 | 5 | 148206641 | 180687768 | 0.2452 | 0.9996 | 3.0006 | 0.9984 | 3 | 1 |
| WCM243_X1_1 | 6 | 203546 | 159420735 | −0.0433 | 0.6142 | 2.0052 | 1.0563 | 2 | 1 |
| WCM243_X1_1 | 6 | 159421004 | 170893406 | −0.4224 | 0.0583 | 2.0688 | 0.0136 | 2 | 0 |
| WCM243_X1_1 | 7 | 193222 | 63529950 | 0.2434 | 0.9974 | 3.0118 | 0.981 | 3 | 1 |
| WCM243_X1_1 | 7 | 63537737 | 100549579 | 0.4837 | 1.3014 | 4.0333 | 0.8959 | 4 | 1 |
| WCM243_X1_1 | 7 | 100549873 | 100634157 | 0.5931 | 1.4356 | 3.4194 | 1.9905 | 3 | 2 |
| WCM243_X1_1 | 7 | 100634374 | 158937185 | 0.4995 | 1.3209 | 3.9701 | 1.0265 | 4 | 1 |
| WCM243_X1_1 | 8 | 190900 | 32621630 | −0.0484 | 0.6072 | 2.0151 | 1.0315 | 2 | 1 |
| WCM243_X1_1 | 8 | 33230211 | 146279483 | 0.2465 | 1.0014 | 2.0227 | 1.9812 | 2 | 2 |
| WCM243_X1_1 | 9 | 116836 | 12704701 | −0.963 | −0.9095 | 1.0452 | 0.0196 | 1 | 0 |
| WCM243_X1_1 | 9 | 12708079 | 105767269 | −0.4227 | 0.0579 | 1.9984 | 0.0835 | 2 | 0 |
| WCM243_X1_1 | 9 | 105767545 | 141111414 | −0.0546 | 0.5985 | 3.0356 | −0.0073 | 3 | 0 |
| WCM243_X1_1 | X | 2707735 | 27999355 | −0.4791 | −0.0314 | NA | NA | NA | NA |
| WCM243_X1_1 | X | 28807508 | 57020905 | 0.2007 | 0.9419 | NA | NA | NA | NA |
| WCM243_X1_1 | X | 57021244 | 65486396 | 0.6779 | 1.5382 | NA | NA | NA | NA |
| WCM243_X1_1 | X | 65817849 | 68060428 | 3.0388 | 4.1168 | NA | NA | NA | NA |
| WCM243_X1_1 | X | 68381320 | 80001126 | 0.1948 | 0.9341 | NA | NA | NA | NA |
| WCM243_X1_1 | X | 80049168 | 154509348 | −0.4666 | −0.0113 | NA | NA | NA | NA |
| WCM247 | 1 | 17483 | 249213973 | −0.0369 | −0.1679 | 0.8901 | 0.8901 | 1 | 1 |
| WCM247 | 10 | 226021 | 135477913 | −0.0636 | −0.9285 | 0.5254 | 0.5254 | 1 | 1 |
| WCM247 | 11 | 193096 | 134605849 | 0.0055 | 0.5699 | 1.4844 | 1.4844 | 1 | 1 |
| WCM247 | 12 | 148474 | 133810710 | −0.0658 | −1.0105 | 0.4964 | 0.4964 | 0 | 0 |
| WCM247 | 13 | 19042005 | 115089411 | −0.1043 | NA | −0.0164 | −0.0164 | 0 | 0 |
| WCM247 | 14 | 19110245 | 22371472 | −0.0242 | 0.0929 | 1.0665 | 1.0665 | 1 | 1 |
| WCM247 | 14 | 22372638 | 22981823 | 0.5819 | 3.529 | 11.5437 | 11.5437 | 12 | 12 |
| WCM247 | 14 | 22982965 | 107283270 | −0.0212 | 0.1478 | 1.1079 | 1.1079 | 1 | 1 |
| WCM247 | 15 | 20083910 | 102506269 | −8.00E−04 | 0.4794 | 1.3941 | 1.3941 | 1 | 1 |
| WCM247 | 16 | 64429 | 90168697 | −0.0446 | −0.3496 | 0.7848 | 0.7848 | 1 | 1 |
| WCM247 | 17 | 131595 | 81187901 | −0.0997 | −4.5411 | 0.043 | 0.043 | 0 | 0 |
| WCM247 | 18 | 47609 | 78005240 | −0.0302 | −0.0238 | 0.9836 | 0.9836 | 1 | 1 |
| WCM247 | 19 | 105041 | 59093696 | −0.0175 | 0.2134 | 1.1594 | 1.1594 | 1 | 1 |
| WCM247 | 2 | 41623 | 243052713 | −0.0769 | −1.5291 | 0.3465 | 0.3465 | 0 | 0 |
| WCM247 | 20 | 68319 | 62934592 | −0.0247 | 0.0835 | 1.0596 | 1.0596 | 1 | 1 |
| WCM247 | 21 | 9590334 | 48084177 | −0.0557 | −0.6578 | 0.6339 | 0.6339 | 1 | 1 |
| WCM247 | 22 | 16159014 | 51216306 | −0.0521 | −0.5524 | 0.6819 | 0.6819 | 1 | 1 |
| WCM247 | 3 | 239495 | 197896740 | −0.0067 | 0.3914 | 1.3116 | 1.3116 | 1 | 1 |
| WCM247 | 4 | 53423 | 190878508 | −0.0297 | −0.0148 | 0.9898 | 0.9898 | 1 | 1 |
| WCM247 | 5 | 90714 | 180687861 | −0.0082 | 0.3683 | 1.2909 | 1.2909 | 1 | 1 |
| WCM247 | 6 | 203397 | 170892918 | −0.0614 | −0.8486 | 0.5553 | 0.5553 | 1 | 1 |
| WCM247 | 7 | 193245 | 330721 | 0.174 | 2.0076 | NA | NA | NA | NA |
| WCM247 | 7 | 538249 | 27191872 | −0.1025 | −7.1518 | 0.007 | 0.007 | 0 | 0 |
| WCM247 | 7 | 27192556 | 158937074 | 0.0894 | 1.4387 | 2.7108 | 2.7108 | 3 | 3 |
| WCM247 | 8 | 163474 | 146279480 | 0.0536 | 1.1233 | 2.1785 | 2.1785 | 2 | 2 |
| WCM247 | 9 | 14885 | 141071710 | −0.022 | 0.1346 | 1.0978 | 1.0978 | 1 | 1 |
| WCM247 | X | 2700148 | 154493576 | 0.0177 | 0.7289 | NA | NA | NA | NA |
| WCM247_Z9_1 | 1 | 721476 | 7886717 | −0.0382 | −0.3994 | 1.0513 | 0.4651 | 1 | 0 |
| WCM247_Z9_1 | 1 | 7887421 | 11172909 | −0.4713 | −2.0315 | 0.3355 | 0.1537 | 0 | 0 |
| WCM247_Z9_1 | 1 | 11174386 | 46088946 | −0.0431 | −0.4122 | 1.0021 | 0.5008 | 1 | 1 |
| WCM247_Z9_1 | 1 | 46089316 | 46120340 | −0.2364 | −0.9887 | NA | NA | NA | NA |
| WCM247_Z9_1 | 1 | 46120949 | 87597961 | −0.4639 | −1.9878 | 0.5101 | −0.0059 | 1 | 0 |
| WCM247_Z9_1 | 1 | 87598225 | 109479978 | −0.2591 | −1.0678 | 0.9614 | −0.0072 | 1 | 0 |
| WCM247_Z9_1 | 1 | 109482344 | 214781892 | 0.1314 | 0.0074 | 1.0386 | 0.9718 | 1 | 1 |
| WCM247_Z9_1 | 1 | 214782135 | 243736289 | −0.2586 | −1.0659 | 0.9842 | −0.0289 | 1 | 0 |
| WCM247_Z9_1 | 1 | 243809291 | 249212354 | 0.167 | 0.0852 | 1.086 | 1.0357 | 1 | 1 |
| WCM247_Z9_1 | 10 | 225988 | 76928356 | −0.2417 | −1.0068 | 0.5166 | 0.4787 | 1 | 0 |
| WCM247_Z9_1 | 10 | 76935848 | 135053292 | −0.0487 | −0.4271 | 1.0053 | 0.4823 | 1 | 0 |
| WCM247_Z9_1 | 10 | 135053476 | 135440108 | −0.2645 | −1.0871 | NA | NA | NA | NA |
| WCM247_Z9_1 | 11 | 193112 | 65484441 | 0.1353 | 0.0161 | 1.0246 | 0.9978 | 1 | 1 |
| WCM247_Z9_1 | 11 | 65486119 | 134856536 | 0.2565 | 0.2713 | 1.2458 | 1.1681 | 1 | 1 |
| WCM247_Z9_1 | 12 | 148637 | 133810896 | −0.0498 | −0.4299 | 0.9859 | 0.4987 | 1 | 0 |
| WCM247_Z9_1 | 13 | 19600515 | 115089411 | −0.2437 | −1.0137 | 0.9762 | 0.0144 | 1 | 0 |
| WCM247_Z9_1 | 14 | 19377703 | 107283184 | 0.1302 | 0.0049 | 1.0034 | 1.0034 | 1 | 1 |
| WCM247_Z9_1 | 15 | 20170150 | 102506276 | −0.0937 | −0.549 | 1.1123 | 0.2547 | 1 | 0 |
| WCM247_Z9_1 | 16 | 66926 | 90233622 | −0.0419 | −0.409 | 1.0001 | 0.5062 | 1 | 1 |
| WCM247_Z9_1 | 17 | 131595 | 44117119 | −0.2392 | −0.9983 | 0.9933 | 0.0079 | 1 | 0 |
| WCM247_Z9_1 | 17 | 44128049 | 47656367 | 0.1394 | 0.0253 | 1.1543 | 0.8811 | 1 | 1 |
| WCM247_Z9_1 | 17 | 47656511 | 80136385 | −0.2309 | −0.9697 | 1.0287 | −0.0075 | 1 | 0 |
| WCM247_Z9_1 | 17 | 80137041 | 81083567 | 0.1098 | −0.0407 | 0.9919 | 0.9524 | 1 | 1 |
| WCM247_Z9_1 | 18 | 48220 | 22907639 | 0.1169 | −0.0249 | 1.0116 | 0.9542 | 1 | 1 |
| WCM247_Z9_1 | 18 | 22930877 | 78005219 | −0.0624 | −0.4635 | 0.9083 | 0.5421 | 1 | 1 |
| WCM247_Z9_1 | 19 | 281483 | 42741075 | 0.1228 | −0.0115 | 1.0142 | 0.9699 | 1 | 1 |
| WCM247_Z9_1 | 19 | 42744173 | 52468136 | −0.2508 | −1.0385 | 0.9859 | −0.0122 | 1 | 0 |
| WCM247_Z9_1 | 19 | 52468520 | 59083968 | 0.1365 | 0.0187 | 1.0309 | 0.9952 | 1 | 1 |
| WCM247_Z9_1 | 2 | 41609 | 128015197 | −0.2411 | −1.0049 | 1.0076 | −0.011 | 1 | 0 |
| WCM247_Z9_1 | 2 | 128016945 | 242946597 | −0.0425 | −0.4105 | 0.9992 | 0.5055 | 1 | 1 |
| WCM247_Z9_1 | 20 | 68351 | 1895842 | −0.443 | −1.8697 | 0.4606 | 0.0866 | 0 | 0 |
| WCM247_Z9_1 | 20 | 1896055 | 25397817 | −0.0582 | −0.4522 | 0.7681 | 0.6938 | 1 | 1 |
| WCM247_Z9_1 | 20 | 25398790 | 62904845 | 0.097 | −0.0699 | 1.4377 | 0.4677 | 1 | 0 |
| WCM247_Z9_1 | 21 | 9907304 | 48084210 | −0.0517 | −0.4348 | 0.9949 | 0.4847 | 1 | 0 |
| WCM247_Z9_1 | 22 | 17060362 | 51219138 | −0.2405 | −1.0026 | 1.0568 | −0.0586 | 1 | 0 |
| WCM247_Z9_1 | 3 | 239431 | 197894726 | 0.1189 | −0.0202 | 0.9994 | 0.9729 | 1 | 1 |
| WCM247_Z9_1 | 4 | 59385 | 190426459 | −0.0384 | −0.3999 | 1.0034 | 0.5124 | 1 | 1 |
| WCM247_Z9_1 | 5 | 140480 | 107559900 | 0.1206 | −0.0164 | 1.0152 | 0.9622 | 1 | 1 |
| WCM247_Z9_1 | 5 | 107684154 | 112222787 | −0.244 | −1.0149 | NA | NA | NA | NA |
| WCM247_Z9_1 | 5 | 112227443 | 180687766 | 0.1238 | −0.0093 | 1.0202 | 0.967 | 1 | 1 |
| WCM247_Z9_1 | 6 | 203577 | 170893409 | −0.0461 | −0.42 | 0.9937 | 0.5012 | 1 | 1 |
| WCM247_Z9_1 | 7 | 193205 | 24331286 | −0.2441 | −1.0149 | 1.0203 | −0.0306 | 1 | 0 |
| WCM247_Z9_1 | 7 | 24663325 | 77584192 | 0.3003 | 0.3582 | 2.5589 | 0.0048 | 3 | 0 |
| WCM247_Z9_1 | 7 | 77648671 | 157208739 | 0.2901 | 0.3383 | 2.0127 | 0.5158 | 2 | 1 |
| WCM247_Z9_1 | 7 | 157234015 | 158937161 | 0.3558 | 0.4647 | NA | NA | NA | NA |
| WCM247_Z9_1 | 8 | 190907 | 81605336 | 0.1291 | 0.0025 | 1.024 | 0.9795 | 1 | 1 |
| WCM247_Z9_1 | 8 | 81733727 | 146279480 | 0.502 | 0.7291 | 2.2249 | 1.0903 | 2 | 1 |
| WCM247_Z9_1 | 9 | 17110 | 20764960 | −0.0184 | −0.3482 | 1.5646 | 0.0065 | 2 | 0 |
| WCM247_Z9_1 | 9 | 20770103 | 23693476 | −0.6799 | −4.4062 | NA | NA | NA | NA |
| WCM247_Z9_1 | 9 | 23701514 | 141111414 | −0.0297 | −0.3773 | 1.4851 | 0.0547 | 1 | 0 |
| WCM247_Z9_1 | X | 2700140 | 154509348 | 0.0827 | −0.1026 | NA | NA | NA | NA |
| WCM252 | 1 | 14930 | 2408066 | 0.0597 | 0.0631 | 1.0462 | 1.0433 | 1 | 1 |
| WCM252 | 1 | 2409892 | 11097930 | −0.5608 | −0.8795 | 0.9874 | 0.0997 | 1 | 0 |
| WCM252 | 1 | 11102891 | 183881247 | 0.0365 | 0.0312 | 1.0219 | 1.0219 | 1 | 1 |
| WCM252 | 1 | 183885666 | 200012867 | −0.6089 | −0.9629 | 0.9496 | 0.0765 | 1 | 0 |
| WCM252 | 1 | 200014657 | 249213961 | 0.0322 | 0.0252 | 1.0176 | 1.0176 | 1 | 1 |
| WCM252 | 10 | 226021 | 135438935 | 0.0111 | −0.0041 | 0.9972 | 0.9972 | 1 | 1 |
| WCM252 | 11 | 193045 | 70387547 | 0.0214 | 0.0103 | 1.0072 | 1.0072 | 1 | 1 |
| WCM252 | 11 | 70449957 | 81601884 | −0.5284 | −0.8243 | 1.0197 | 0.1099 | 1 | 0 |
| WCM252 | 11 | 82444623 | 103175488 | −0.0097 | −0.033 | 0.9774 | 0.9774 | 1 | 1 |
| WCM252 | 11 | 103178529 | 110001824 | −0.5513 | −0.8632 | 0.9984 | 0.1011 | 1 | 0 |
| WCM252 | 11 | 110007492 | 132307150 | 0.0452 | 0.0431 | 1.0303 | 1.0303 | 1 | 1 |
| WCM252 | 11 | 132399108 | 134184202 | −0.5429 | −0.8488 | 1.0066 | 0.104 | 1 | 0 |
| WCM252 | 11 | 134188671 | 134605916 | 0.0839 | 0.0962 | NA | NA | NA | NA |
| WCM252 | 12 | 148430 | 111319152 | 0.417 | 0.5337 | 1.8039 | 1.0913 | 2 | 1 |
| WCM252 | 12 | 111321916 | 133683261 | −0.0238 | −0.0528 | 1.8381 | 0.0901 | 2 | 0 |
| WCM252 | 12 | 133697006 | 133810703 | 0.5676 | 0.7222 | 2.0268 | 1.2726 | 2 | 1 |
| WCM252 | 13 | 19042004 | 43155462 | 0.0026 | −0.0159 | 0.9891 | 0.9891 | 1 | 1 |
| WCM252 | 13 | 43175059 | 115089411 | −0.575 | −0.9039 | 0.9389 | 0.13 | 1 | 0 |
| WCM252 | 14 | 19110187 | 107283270 | 0.0182 | 0.0058 | 1.004 | 1.004 | 1 | 1 |
| WCM252 | 15 | 20083896 | 102516227 | 0.0353 | 0.0295 | 1.0207 | 1.0207 | 1 | 1 |
| WCM252 | 16 | 67591 | 90168693 | −0.0034 | −0.0243 | 0.9833 | 0.9833 | 1 | 1 |
| WCM252 | 17 | 131595 | 3716289 | −0.018 | −0.0446 | 0.9696 | 0.9696 | 1 | 1 |
| WCM252 | 17 | 3716581 | 16614960 | −0.5885 | −0.9273 | 1.0061 | 0.0456 | 1 | 0 |
| WCM252 | 17 | 16623497 | 56357410 | 0.0136 | −5.00E−04 | 0.9996 | 0.9996 | 1 | 1 |
| WCM252 | 17 | 56357750 | 56604390 | −0.4643 | −0.7176 | 1.0797 | 0.1365 | 1 | 0 |
| WCM252 | 17 | 56604562 | 81187930 | 0.0173 | 0.0045 | 1.0032 | 1.0032 | 1 | 1 |
| WCM252 | 18 | 47808 | 8406478 | 0.0148 | 0.0011 | 1.0008 | 1.0008 | 1 | 1 |
| WCM252 | 18 | 8609854 | 10871272 | −0.6316 | −1.0031 | 0.9162 | 0.0817 | 1 | 0 |
| WCM252 | 18 | 10871415 | 11874986 | −0.2733 | −0.4165 | NA | NA | NA | NA |
| WCM252 | 18 | 11876613 | 74968072 | 0.0167 | 0.0038 | 1.0026 | 1.0026 | 1 | 1 |
| WCM252 | 18 | 74980707 | 78005123 | −0.4993 | −0.7755 | 1.1009 | 0.0675 | 1 | 0 |
| WCM252 | 19 | 105041 | 4675262 | −0.5641 | −0.8851 | 1.0122 | 0.0707 | 1 | 0 |
| WCM252 | 19 | 4676683 | 24345254 | 0.0241 | 0.014 | 1.0097 | 1.0097 | 1 | 1 |
| WCM252 | 19 | 29213655 | 31769738 | 2.1061 | 2.468 | NA | NA | NA | NA |
| WCM252 | 19 | 31770009 | 33701820 | −0.5519 | −0.8641 | 0.9632 | 0.1356 | 1 | 0 |
| WCM252 | 19 | 33702121 | 34513619 | 2.1489 | 2.5138 | NA | NA | NA | NA |
| WCM252 | 19 | 34583530 | 38055958 | −0.5754 | −0.9046 | 0.969 | 0.0994 | 1 | 0 |
| WCM252 | 19 | 38056225 | 38397909 | 2.1984 | 2.5666 | 10.3749 | 1.4735 | 10 | 1 |
| WCM252 | 19 | 38572055 | 39384115 | −0.5244 | −0.8176 | 1.0188 | 0.116 | 1 | 0 |
| WCM252 | 19 | 39384471 | 41399799 | 2.4183 | 2.7998 | 12.897 | 1.0301 | 13 | 1 |
| WCM252 | 19 | 41404212 | 48973910 | −0.5396 | −0.8433 | 1.0257 | 0.089 | 1 | 0 |
| WCM252 | 19 | 48976609 | 59093696 | 0.0087 | −0.0073 | 0.9949 | 0.9949 | 1 | 1 |
| WCM252 | 2 | 41614 | 61025160 | −0.0065 | −0.0285 | 0.9804 | 0.9804 | 1 | 1 |
| WCM252 | 2 | 61025409 | 62739000 | −0.6492 | −1.0345 | NA | NA | NA | NA |
| WCM252 | 2 | 62901154 | 127821611 | −0.0019 | −0.0221 | 0.9848 | 0.9848 | 1 | 1 |
| WCM252 | 2 | 127825802 | 128583801 | −0.5502 | −0.8613 | 1.0549 | 0.046 | 1 | 0 |
| WCM252 | 2 | 128587255 | 128947505 | 0.0453 | 0.0433 | 1.0305 | 1.0305 | 1 | 1 |
| WCM252 | 2 | 129025896 | 131672145 | −0.5855 | −0.9221 | NA | NA | NA | NA |
| WCM252 | 2 | 131672309 | 179843346 | 0.0075 | −0.009 | 0.9938 | 0.9938 | 1 | 1 |
| WCM252 | 2 | 179912073 | 193059229 | −0.5673 | −0.8906 | 1.0082 | 0.0706 | 1 | 0 |
| WCM252 | 2 | 193059496 | 219960128 | 0.0364 | 0.031 | 1.0217 | 1.0217 | 1 | 1 |
| WCM252 | 2 | 219976528 | 229035272 | −0.5943 | −0.9373 | 0.9231 | 0.1213 | 1 | 0 |
| WCM252 | 2 | 229035294 | 233439024 | 0.0461 | 0.0444 | 1.0312 | 1.0312 | 1 | 1 |
| WCM252 | 2 | 233445900 | 238233373 | −0.5092 | −0.7919 | 1.0395 | 0.1156 | 1 | 0 |
| WCM252 | 2 | 238234260 | 243037022 | 0.0235 | 0.0131 | 1.0091 | 1.0091 | 1 | 1 |
| WCM252 | 20 | 68446 | 62934592 | 0.0079 | −0.0085 | 0.9941 | 0.9941 | 1 | 1 |
| WCM252 | 21 | 9590292 | 39891572 | −0.0324 | −0.0648 | 0.956 | 0.956 | 1 | 1 |
| WCM252 | 21 | 39903359 | 42866713 | −0.6276 | −0.9959 | 0.8994 | 0.1035 | 1 | 0 |
| WCM252 | 21 | 42870049 | 48084142 | 0.0332 | 0.0266 | 1.0186 | 1.0186 | 1 | 1 |
| WCM252 | 22 | 16159043 | 51223730 | 0.0107 | −0.0047 | 0.9968 | 0.9968 | 1 | 1 |
| WCM252 | 3 | 239524 | 44884660 | 0.026 | 0.0166 | 1.0116 | 1.0116 | 1 | 1 |
| WCM252 | 3 | 44889548 | 90306304 | −0.5739 | −0.9019 | 0.9944 | 0.0759 | 1 | 0 |
| WCM252 | 3 | 93593059 | 176769541 | 0.4419 | 0.5651 | 1.9466 | 1.0124 | 2 | 1 |
| WCM252 | 3 | 176771755 | 184429374 | −0.0127 | −0.0372 | 0.9746 | 0.9746 | 1 | 1 |
| WCM252 | 3 | 184542422 | 197896678 | 0.4523 | 0.5783 | 1.9843 | 1.0019 | 2 | 1 |
| WCM252 | 4 | 53344 | 2097555 | −0.033 | −0.0657 | 0.9555 | 0.9555 | 1 | 1 |
| WCM252 | 4 | 2129996 | 5969113 | −0.432 | −0.6651 | 1.0486 | 0.2126 | 1 | 0 |
| WCM252 | 4 | 5975428 | 190903980 | −0.0016 | −0.0217 | 0.985 | 0.985 | 1 | 1 |
| WCM252 | 5 | 90750 | 45695959 | 0.0088 | −0.0072 | 0.995 | 0.995 | 1 | 1 |
| WCM252 | 5 | 49694840 | 68541847 | −0.5939 | −0.9367 | 0.9771 | 0.0677 | 1 | 0 |
| WCM252 | 5 | 68546612 | 118729017 | −0.0013 | −0.0213 | 0.9853 | 0.9853 | 1 | 1 |
| WCM252 | 5 | 118792001 | 132270597 | −0.5725 | −0.8996 | 0.9661 | 0.1059 | 1 | 0 |
| WCM252 | 5 | 132272804 | 153374509 | 1.00E−04 | −0.0194 | 0.9866 | 0.9866 | 1 | 1 |
| WCM252 | 5 | 153377398 | 154346445 | −0.5166 | −0.8045 | NA | NA | NA | NA |
| WCM252 | 5 | 154393462 | 180687821 | 0.0291 | 0.0209 | 1.0146 | 1.0146 | 1 | 1 |
| WCM252 | 6 | 203428 | 24353742 | −0.0161 | −0.042 | 0.9713 | 0.9713 | 1 | 1 |
| WCM252 | 6 | 24357648 | 26225341 | −0.6319 | −1.0036 | 0.9559 | 0.0416 | 1 | 0 |
| WCM252 | 6 | 26225743 | 165809769 | 0.0182 | 0.0059 | 1.0041 | 1.0041 | 1 | 1 |
| WCM252 | 6 | 165826966 | 170892855 | −0.6232 | −0.9881 | 0.8971 | 0.1112 | 1 | 0 |
| WCM252 | 7 | 193205 | 1474342 | −0.0905 | −0.1473 | 0.9029 | 0.9029 | 1 | 1 |
| WCM252 | 7 | 1474799 | 4218082 | −0.4688 | −0.7251 | 1.078 | 0.1319 | 1 | 0 |
| WCM252 | 7 | 4226333 | 7677510 | −0.0181 | −0.0448 | 0.9694 | 0.9694 | 1 | 1 |
| WCM252 | 7 | 7678680 | 16505341 | −0.6059 | −0.9577 | 0.9604 | 0.0693 | 1 | 0 |
| WCM252 | 7 | 16566645 | 18166885 | −1.6706 | −4.5338 | NA | NA | NA | NA |
| WCM252 | 7 | 18330068 | 20706356 | −0.6114 | −0.9673 | 0.9382 | 0.0847 | 1 | 0 |
| WCM252 | 7 | 20721116 | 53879290 | 0.0265 | 0.0173 | 1.0121 | 1.0121 | 1 | 1 |
| WCM252 | 7 | 53879540 | 62708584 | −0.6485 | −1.0334 | 0.9317 | 0.0454 | 1 | 0 |
| WCM252 | 7 | 62752657 | 107303886 | 0.0089 | −0.0071 | 0.9978 | 0.9923 | 1 | 1 |
| WCM252 | 7 | 107312607 | 108524593 | −0.5895 | −0.929 | NA | NA | NA | NA |
| WCM252 | 7 | 109599655 | 154989982 | 0.0745 | 0.0834 | 1.0595 | 1.0595 | 1 | 1 |
| WCM252 | 7 | 155014996 | 158937161 | −0.1936 | −0.2971 | 1.1061 | 0.5216 | 1 | 1 |
| WCM252 | 8 | 163486 | 23160868 | −0.0298 | −0.0612 | 0.9584 | 0.9584 | 1 | 1 |
| WCM252 | 8 | 23167191 | 28205909 | −0.5517 | −0.8639 | 0.9914 | 0.1075 | 1 | 0 |
| WCM252 | 8 | 28206254 | 48068897 | 0.0027 | −0.0157 | 0.9892 | 0.9892 | 1 | 1 |
| WCM252 | 8 | 48069064 | 59361259 | −0.6043 | −0.9549 | 0.9683 | 0.0635 | 1 | 0 |
| WCM252 | 8 | 59363647 | 60032851 | −1.5944 | −3.9082 | 0.1345 | −0.0013 | 0 | 0 |
| WCM252 | 8 | 60882984 | 70476343 | −0.5857 | −0.9225 | 0.94 | 0.1152 | 1 | 0 |
| WCM252 | 8 | 70488148 | 146279431 | 0.0282 | 0.0197 | 1.0138 | 1.0138 | 1 | 1 |
| WCM252 | 9 | 15902 | 33750592 | −0.571 | −0.8969 | 0.9472 | 0.1268 | 1 | 0 |
| WCM252 | 9 | 33784581 | 35555392 | 0.041 | 0.0374 | 1.0729 | 0.9795 | 1 | 1 |
| WCM252 | 9 | 35555914 | 87636359 | −0.5571 | −0.873 | 0.963 | 0.129 | 1 | 0 |
| WCM252 | 9 | 88162019 | 141107525 | 0.0419 | 0.0385 | 1.0271 | 1.0271 | 1 | 1 |
| WCM252 | X | 2700122 | 154493601 | 0.0354 | 0.0297 | NA | NA | NA | NA |
| WCM252_Z3_1 | 1 | 721450 | 2341812 | 0.0125 | 0.0266 | 1.0464 | 0.9909 | 1 | 1 |
| WCM252_Z3_1 | 1 | 2342303 | 11103002 | −0.667 | −1.0085 | 0.9964 | −0.0023 | 1 | 0 |
| WCM252_Z3_1 | 1 | 11103514 | 183895406 | −0.0177 | −0.0146 | 1.0008 | 0.979 | 1 | 1 |
| WCM252_Z3_1 | 1 | 183899402 | 198828264 | −0.6713 | −1.0159 | 1.0572 | −0.0682 | 1 | 0 |
| WCM252_Z3_1 | 1 | 199997036 | 249212344 | −0.0168 | −0.0135 | 1.0029 | 0.9784 | 1 | 1 |
| WCM252_Z3_1 | 10 | 226016 | 135440108 | −0.0194 | −0.0171 | 0.9882 | 0.9882 | 1 | 1 |
| WCM252_Z3_1 | 11 | 193113 | 70349036 | −0.0067 | 4.00E−04 | 1.014 | 0.9866 | 1 | 1 |
| WCM252_Z3_1 | 11 | 70505962 | 81601788 | −0.6665 | −1.0076 | 1.0108 | −0.016 | 1 | 0 |
| WCM252_Z3_1 | 11 | 82443590 | 103229027 | −0.0406 | −0.0462 | 0.9805 | 0.9564 | 1 | 1 |
| WCM252_Z3_1 | 11 | 103270422 | 109294927 | −0.6928 | −1.0538 | NA | NA | NA | NA |
| WCM252_Z3_1 | 11 | 109964156 | 132398997 | −0.0291 | −0.0304 | 0.9859 | 0.9724 | 1 | 1 |
| WCM252_Z3_1 | 11 | 132527171 | 134229035 | −0.5989 | −0.8917 | NA | NA | NA | NA |
| WCM252_Z3_1 | 11 | 134234253 | 134856626 | −0.04 | −0.0455 | NA | NA | NA | NA |
| WCM252_Z3_1 | 12 | 148642 | 111311714 | 0.4136 | 0.5496 | 1.9899 | 0.9375 | 2 | 1 |
| WCM252_Z3_1 | 12 | 111317723 | 133502284 | −0.0079 | −0.0012 | 2.0205 | −0.0221 | 2 | 0 |
| WCM252_Z3_1 | 12 | 133670042 | 133810703 | 0.4627 | 0.6108 | NA | NA | NA | NA |
| WCM252_Z3_1 | 13 | 19600364 | 42891786 | −0.0297 | −0.0311 | 1.007 | 0.9503 | 1 | 1 |
| WCM252_Z3_1 | 13 | 42893359 | 115089411 | −0.661 | −0.9979 | 1.0117 | −0.0103 | 1 | 0 |
| WCM252_Z3_1 | 14 | 19377727 | 107283063 | −0.0103 | −0.0045 | 1.0023 | 0.9914 | 1 | 1 |
| WCM252_Z3_1 | 15 | 20170145 | 102517667 | −0.0171 | −0.0139 | 1.0054 | 0.9755 | 1 | 1 |
| WCM252_Z3_1 | 16 | 67604 | 90162150 | −0.0031 | 0.0053 | 1.0147 | 0.9927 | 1 | 1 |
| WCM252_Z3_1 | 17 | 131595 | 3680928 | 0.0133 | 0.0278 | 1.05 | 0.9889 | 1 | 1 |
| WCM252_Z3_1 | 17 | 3704410 | 16594009 | −0.6581 | −0.9929 | 1.0233 | −0.0183 | 1 | 0 |
| WCM252_Z3_1 | 17 | 16594048 | 56350247 | −0.0106 | −0.0049 | 1.0116 | 0.9816 | 1 | 1 |
| WCM252_Z3_1 | 17 | 56350921 | 56599428 | −0.599 | −0.8918 | 1.0079 | 0.07 | 1 | 0 |
| WCM252_Z3_1 | 17 | 56602496 | 81083561 | 0.0022 | 0.0126 | 1.0143 | 1.0033 | 1 | 1 |
| WCM252_Z3_1 | 18 | 48341 | 8394552 | 0.0051 | 0.0165 | 1.0267 | 0.9963 | 1 | 1 |
| WCM252_Z3_1 | 18 | 8406156 | 10871384 | −0.6217 | −0.9303 | 1.0472 | 0.0023 | 1 | 0 |
| WCM252_Z3_1 | 18 | 10979629 | 74980815 | −0.021 | −0.0192 | 0.9868 | 0.9868 | 1 | 1 |
| WCM252_Z3_1 | 18 | 76856583 | 78005199 | −0.5668 | −0.8379 | NA | NA | NA | NA |
| WCM252_Z3_1 | 19 | 281503 | 4660726 | −0.6438 | −0.9682 | 1.0195 | 0.0028 | 1 | 0 |
| WCM252_Z3_1 | 19 | 4668644 | 24310661 | −0.0085 | −0.002 | 1.0054 | 0.9917 | 1 | 1 |
| WCM252_Z3_1 | 19 | 29567134 | 31769483 | 2.9223 | 3.3313 | NA | NA | NA | NA |
| WCM252_Z3_1 | 19 | 31769763 | 33702026 | −0.6675 | −1.0093 | 0.9934 | 2.00E−04 | 1 | 0 |
| WCM252_Z3_1 | 19 | 33702390 | 34513633 | 2.8041 | 3.2086 | NA | NA | NA | NA |
| WCM252_Z3_1 | 19 | 34513852 | 38055827 | −0.6926 | −1.0533 | 0.9665 | −0.0028 | 1 | 0 |
| WCM252_Z3_1 | 19 | 38056158 | 38388864 | 2.7631 | 3.1659 | NA | NA | NA | NA |
| WCM252_Z3_1 | 19 | 38572343 | 39382581 | −0.6483 | −0.9759 | 1.0143 | 0.0025 | 1 | 0 |
| WCM252_Z3_1 | 19 | 39384564 | 40275185 | 2.9418 | 3.3515 | 19.3875 | 1.0263 | 19 | 1 |
| WCM252_Z3_1 | 19 | 40276461 | 40978590 | 3.4077 | 3.8317 | 27.649 | 0.828 | 28 | 1 |
| WCM252_Z3_1 | 19 | 40993650 | 41404085 | 2.7532 | 3.1555 | 17.0498 | 0.7716 | 17 | 1 |
| WCM252_Z3_1 | 19 | 41405962 | 48968015 | −0.6711 | −1.0155 | 0.9848 | 0.0045 | 1 | 0 |
| WCM252_Z3_1 | 19 | 48973263 | 59083985 | −0.0303 | −0.0321 | 0.9888 | 0.9672 | 1 | 1 |
| WCM252_Z3_1 | 2 | 41612 | 61019325 | −0.0306 | −0.0325 | 0.9926 | 0.9629 | 1 | 1 |
| WCM252_Z3_1 | 2 | 61020808 | 62738946 | −0.6591 | −0.9947 | NA | NA | NA | NA |
| WCM252_Z3_1 | 2 | 62934328 | 127826533 | −0.0203 | −0.0182 | 1.0119 | 0.963 | 1 | 1 |
| WCM252_Z3_1 | 2 | 127827580 | 131595261 | −0.5579 | −0.8232 | 1.0822 | 0.0481 | 1 | 0 |
| WCM252_Z3_1 | 2 | 131671631 | 179843334 | −0.003 | 0.0055 | 1.0175 | 0.9901 | 1 | 1 |
| WCM252_Z3_1 | 2 | 179912030 | 196545161 | −0.6648 | −1.0047 | 0.982 | 0.0148 | 1 | 0 |
| WCM252_Z3_1 | 2 | 196545513 | 219959996 | −0.0133 | −0.0086 | 1.0254 | 0.9627 | 1 | 1 |
| WCM252_Z3_1 | 2 | 220011408 | 228993243 | −0.6508 | −0.9803 | 1.0171 | −0.0034 | 1 | 0 |
| WCM252_Z3_1 | 2 | 228996781 | 233438805 | −0.0025 | 0.0062 | 1.1005 | 0.9081 | 1 | 1 |
| WCM252_Z3_1 | 2 | 233445763 | 237300993 | −0.6493 | −0.9777 | 1.0274 | −0.0118 | 1 | 0 |
| WCM252_Z3_1 | 2 | 237349734 | 242946604 | 0.0105 | 0.0239 | 1.025 | 1.0084 | 1 | 1 |
| WCM252_Z3_1 | 20 | 68375 | 62904847 | −0.0123 | −0.0072 | 1.0018 | 0.9882 | 1 | 1 |
| WCM252_Z3_1 | 21 | 9826205 | 39817361 | −0.0263 | −0.0265 | 1.0116 | 0.9521 | 1 | 1 |
| WCM252_Z3_1 | 21 | 39818741 | 43134216 | −0.706 | −1.0772 | 0.9617 | −0.0138 | 1 | 0 |
| WCM252_Z3_1 | 21 | 43134276 | 48084282 | −0.0237 | −0.023 | 0.9977 | 0.9707 | 1 | 1 |
| WCM252_Z3_1 | 22 | 16287467 | 51215142 | −0.0182 | −0.0153 | 1.0003 | 0.9785 | 1 | 1 |
| WCM252_Z3_1 | 3 | 239405 | 44893778 | −0.0037 | 0.0045 | 1.0031 | 1.0031 | 1 | 1 |
| WCM252_Z3_1 | 3 | 44903385 | 89521693 | −0.6605 | −0.9972 | 0.9913 | 0.0107 | 1 | 0 |
| WCM252_Z3_1 | 3 | 89528644 | 176750860 | 0.4149 | 0.5512 | 1.9424 | 0.9882 | 2 | 1 |
| WCM252_Z3_1 | 3 | 176756215 | 184550501 | 0.0031 | 0.0138 | 1.0496 | 0.9696 | 1 | 1 |
| WCM252_Z3_1 | 3 | 184552483 | 197894719 | 0.4285 | 0.5682 | 1.9995 | 0.9658 | 2 | 1 |
| WCM252_Z3_1 | 4 | 59331 | 2087469 | 0.0108 | 0.0243 | 1.0267 | 1.0073 | 1 | 1 |
| WCM252_Z3_1 | 4 | 2097647 | 2515531 | −0.6307 | −0.9457 | NA | NA | NA | NA |
| WCM252_Z3_1 | 4 | 2598055 | 3088697 | −0.0238 | −0.0231 | NA | NA | NA | NA |
| WCM252_Z3_1 | 4 | 3101043 | 5894571 | −0.6548 | −0.9871 | 0.9233 | 0.0857 | 1 | 0 |
| WCM252_Z3_1 | 4 | 5925054 | 190426570 | −0.0156 | −0.0118 | 1.0068 | 0.9769 | 1 | 1 |
| WCM252_Z3_1 | 5 | 140532 | 49699283 | −0.0208 | −0.019 | 1.0018 | 0.972 | 1 | 1 |
| WCM252_Z3_1 | 5 | 49706767 | 68550493 | −0.6732 | −1.0192 | 0.9699 | 0.0169 | 1 | 0 |
| WCM252_Z3_1 | 5 | 68551401 | 118729066 | −0.0275 | −0.0281 | 0.9982 | 0.9631 | 1 | 1 |
| WCM252_Z3_1 | 5 | 118788464 | 132270123 | −0.6614 | −0.9987 | 0.9382 | 0.0627 | 1 | 0 |
| WCM252_Z3_1 | 5 | 132270461 | 153190635 | −0.0155 | −0.0116 | 0.992 | 0.992 | 1 | 1 |
| WCM252_Z3_1 | 5 | 153372563 | 154336695 | −0.645 | −0.9702 | NA | NA | NA | NA |
| WCM252_Z3_1 | 5 | 154346250 | 180687803 | 0.0032 | 0.0139 | 1.0207 | 0.9986 | 1 | 1 |
| WCM252_Z3_1 | 6 | 203552 | 24357971 | −0.0054 | 0.0023 | 1.018 | 0.9851 | 1 | 1 |
| WCM252_Z3_1 | 6 | 24403380 | 26056549 | −0.6224 | −0.9315 | 1.0608 | −0.0122 | 1 | 0 |
| WCM252_Z3_1 | 6 | 26087705 | 165809845 | −0.0123 | −0.0073 | 1.0059 | 0.984 | 1 | 1 |
| WCM252_Z3_1 | 6 | 165827133 | 170893407 | −0.6579 | −0.9926 | 0.985 | 0.0201 | 1 | 0 |
| WCM252_Z3_1 | 7 | 193451 | 1276154 | 0.01 | 0.0233 | 1.1119 | 0.9206 | 1 | 1 |
| WCM252_Z3_1 | 7 | 1473997 | 4218187 | −0.6271 | −0.9395 | 1.0339 | 0.0089 | 1 | 0 |
| WCM252_Z3_1 | 7 | 4245515 | 7646687 | 0.0022 | 0.0126 | 1.0294 | 0.9881 | 1 | 1 |
| WCM252_Z3_1 | 7 | 7676634 | 16503950 | −0.6108 | −0.9117 | 0.9958 | 0.0673 | 1 | 0 |
| WCM252_Z3_1 | 7 | 16505238 | 18330136 | −1.6571 | −3.9082 | NA | NA | NA | NA |
| WCM252_Z3_1 | 7 | 18498491 | 20698710 | −0.6784 | −1.0284 | NA | NA | NA | NA |
| WCM252_Z3_1 | 7 | 20706390 | 53104210 | −0.0055 | 0.0021 | 1.0138 | 0.9891 | 1 | 1 |
| WCM252_Z3_1 | 7 | 54610425 | 57529635 | −0.6534 | −0.9848 | NA | NA | NA | NA |
| WCM252_Z3_1 | 7 | 63529293 | 107303789 | −0.013 | −0.0082 | 1.0121 | 0.9766 | 1 | 1 |
| WCM252_Z3_1 | 7 | 107312675 | 108524568 | −0.5946 | −0.8844 | NA | NA | NA | NA |
| WCM252_Z3_1 | 7 | 109599726 | 157060397 | −0.0027 | 0.0059 | 1.0124 | 0.9959 | 1 | 1 |
| WCM252_Z3_1 | 7 | 157151328 | 158937022 | −0.4036 | −0.5762 | NA | NA | NA | NA |
| WCM252_Z3_1 | 8 | 190907 | 23174487 | −0.0066 | 5.00E−04 | 1.0154 | 0.9853 | 1 | 1 |
| WCM252_Z3_1 | 8 | 23177553 | 28047189 | −0.6624 | −1.0005 | 0.9608 | 0.0389 | 1 | 0 |
| WCM252_Z3_1 | 8 | 28186691 | 47759684 | −0.008 | −0.0014 | 1.0223 | 0.9757 | 1 | 1 |
| WCM252_Z3_1 | 8 | 47842465 | 59360111 | −0.6326 | −0.9489 | 1.0774 | −0.0413 | 1 | 0 |
| WCM252_Z3_1 | 8 | 59404150 | 60031477 | −1.7298 | −4.469 | NA | NA | NA | NA |
| WCM252_Z3_1 | 8 | 61121416 | 69730480 | −0.6413 | −0.9639 | 1.0254 | −1.00E−04 | 1 | 0 |
| WCM252_Z3_1 | 8 | 70476343 | 146279527 | −0.0023 | 0.0064 | 1.0251 | 0.9838 | 1 | 1 |
| WCM252_Z3_1 | 9 | 17079 | 33676526 | −0.6641 | −1.0034 | 1.0105 | −0.0128 | 1 | 0 |
| WCM252_Z3_1 | 9 | 33794809 | 35555075 | −0.037 | −0.0412 | NA | NA | NA | NA |
| WCM252_Z3_1 | 9 | 35555520 | 88162058 | −0.6553 | −0.988 | 0.9709 | 0.0374 | 1 | 0 |
| WCM252_Z3_1 | 9 | 88190371 | 141111414 | −0.0108 | −0.0052 | 1.0073 | 0.9855 | 1 | 1 |
| WCM252_Z3_1 | X | 2700139 | 154509348 | −0.055 | −0.0661 | NA | NA | NA | NA |
| WCM253 | 1 | 17459 | 249213964 | −0.0055 | 0.1827 | 1.135 | 1.135 | 1 | 1 |
| WCM253 | 10 | 225982 | 135477897 | −0.0271 | −0.0776 | 0.9476 | 0.9476 | 1 | 1 |
| WCM253 | 11 | 193096 | 62327358 | −0.0107 | 0.1239 | 1.0897 | 1.0897 | 1 | 1 |
| WCM253 | 11 | 62327798 | 67377973 | −0.1438 | NA | −0.0201 | −0.0201 | 0 | 0 |
| WCM253 | 11 | 67378574 | 134605959 | −0.01 | 0.1315 | 1.0954 | 1.0954 | 1 | 1 |
| WCM253 | 12 | 148465 | 133810710 | −0.0393 | −0.2474 | 0.8424 | 0.8424 | 1 | 1 |
| WCM253 | 13 | 19041965 | 115089411 | −0.0445 | −0.3258 | 0.7979 | 0.7979 | 1 | 1 |
| WCM253 | 14 | 19110239 | 107283270 | −0.0288 | −0.101 | 0.9324 | 0.9324 | 1 | 1 |
| WCM253 | 15 | 20083896 | 102506282 | −0.0163 | 0.0578 | 1.0409 | 1.0409 | 1 | 1 |
| WCM253 | 16 | 97550 | 90234899 | −0.0377 | −0.2236 | 0.8564 | 0.8564 | 1 | 1 |
| WCM253 | 17 | 131595 | 81052545 | −0.037 | −0.2147 | 0.8617 | 0.8617 | 1 | 1 |
| WCM253 | 18 | 47592 | 78005164 | −0.0328 | −0.1553 | 0.8979 | 0.8979 | 1 | 1 |
| WCM253 | 19 | 105050 | 59093696 | −0.0397 | −0.2529 | 0.8392 | 0.8392 | 1 | 1 |
| WCM253 | 2 | 41620 | 243037116 | −0.0184 | 0.0326 | 1.0229 | 1.0229 | 1 | 1 |
| WCM253 | 20 | 68423 | 62934618 | −0.0037 | 0.2024 | 1.1506 | 1.1506 | 1 | 1 |
| WCM253 | 21 | 9590301 | 48084202 | −0.0375 | −0.2209 | 0.858 | 0.858 | 1 | 1 |
| WCM253 | 22 | 16159014 | 51219090 | 0.0019 | 0.2631 | 1.2001 | 1.2001 | 1 | 1 |
| WCM253 | 3 | 239349 | 197896700 | −0.0178 | 0.0398 | 1.028 | 1.028 | 1 | 1 |
| WCM253 | 4 | 53446 | 190903980 | −0.0256 | −0.0588 | 0.9601 | 0.9601 | 1 | 1 |
| WCM253 | 5 | 90784 | 180687828 | −0.0223 | −0.0158 | 0.9891 | 0.9891 | 1 | 1 |
| WCM253 | 6 | 203561 | 170892848 | −0.0494 | −0.4024 | 0.7566 | 0.7566 | 1 | 1 |
| WCM253 | 7 | 193171 | 158937185 | −0.0267 | −0.0734 | 0.9504 | 0.9504 | 1 | 1 |
| WCM253 | 8 | 163470 | 146279453 | −0.0287 | −0.099 | 0.9337 | 0.9337 | 1 | 1 |
| WCM253 | 9 | 73203 | 141107495 | −0.0081 | 0.1537 | 1.1124 | 1.1124 | 1 | 1 |
| WCM253 | X | 2700163 | 154507335 | −0.0385 | −0.2355 | NA | NA | NA | NA |
| WCM253_Z1_1 | 1 | 69569 | 120468283 | −0.1487 | 0.606 | 2.0585 | 0.9855 | 2 | 1 |
| WCM253_Z1_1 | 1 | 120469231 | 145682077 | 0.6058 | 1.6064 | 4.6306 | 1.4592 | 5 | 1 |
| WCM253_Z1_1 | 1 | 145683579 | 157653025 | 0.4115 | 1.3639 | 3.0346 | 2.1129 | 3 | 2 |
| WCM253_Z1_1 | 1 | 157659649 | 159750941 | −0.0426 | 0.7579 | 2.0132 | 1.369 | 2 | 1 |
| WCM253_Z1_1 | 1 | 159751148 | 198703332 | 0.3478 | 1.2825 | 2.8704 | 1.9947 | 3 | 2 |
| WCM253_Z1_1 | 1 | 198711139 | 215781395 | 0.7002 | 1.7215 | 3.3537 | 3.2419 | 3 | 3 |
| WCM253_Z1_1 | 1 | 215791431 | 249212388 | 0.0699 | 0.9142 | 3.7779 | −0.0088 | 4 | 0 |
| WCM253_Z1_1 | 10 | 255873 | 38679844 | −0.5974 | −0.1057 | 1.9104 | −0.0517 | 2 | 0 |
| WCM253_Z1_1 | 10 | 39147119 | 73122353 | 0.1204 | 0.9829 | 2.0165 | 1.9364 | 2 | 2 |
| WCM253_Z1_1 | 10 | 73157066 | 89312198 | −0.4828 | 0.0891 | 2.0925 | 0.0349 | 2 | 0 |
| WCM253_Z1_1 | 10 | 89419744 | 90580238 | −1.5302 | −2.681 | NA | NA | NA | NA |
| WCM253_Z1_1 | 10 | 90582744 | 135440108 | −0.5154 | 0.0347 | 2.0439 | 0.0048 | 2 | 0 |
| WCM253_Z1_1 | 11 | 193112 | 15263652 | −0.2188 | 0.5027 | 1.8582 | 0.9755 | 2 | 1 |
| WCM253_Z1_1 | 11 | 15267468 | 30928177 | 0.3723 | 1.3139 | 2.9632 | 2.0089 | 3 | 2 |
| WCM253_Z1_1 | 11 | 30930590 | 47823386 | 0.125 | 0.9892 | 2.0065 | 1.9637 | 2 | 2 |
| WCM253_Z1_1 | 11 | 47826104 | 48131610 | 0.4492 | 1.4116 | NA | NA | NA | NA |
| WCM253_Z1_1 | 11 | 48134424 | 64981522 | −0.1072 | 0.6659 | 2.0246 | 1.1485 | 2 | 1 |
| WCM253_Z1_1 | 11 | 64981837 | 73689104 | −0.1489 | 0.6056 | 2.1223 | 0.9209 | 2 | 1 |
| WCM253_Z1_1 | 11 | 73689423 | 74054403 | −0.0234 | 0.7849 | NA | NA | NA | NA |
| WCM253_Z1_1 | 11 | 74056686 | 92087892 | 0.2326 | 1.1327 | 2.4092 | 1.976 | 2 | 2 |
| WCM253_Z1_1 | 11 | 92088270 | 103157068 | −0.1668 | 0.5794 | 2.0678 | 0.9207 | 2 | 1 |
| WCM253_Z1_1 | 11 | 103158316 | 134856562 | −0.5184 | 0.0296 | 2.0176 | 0.024 | 2 | 0 |
| WCM253_Z1_1 | 12 | 148811 | 1967752 | 0.2061 | 1.0977 | 2.1883 | 2.0919 | 2 | 2 |
| WCM253_Z1_1 | 12 | 1969335 | 49434086 | 0.0681 | 0.9119 | 1.9098 | 1.8531 | 2 | 2 |
| WCM253_Z1_1 | 12 | 49434349 | 93881384 | 0.246 | 1.1502 | 2.2661 | 2.1729 | 2 | 2 |
| WCM253_Z1_1 | 12 | 93898134 | 133810874 | −0.1736 | 0.5695 | 3.0027 | −0.0347 | 3 | 0 |
| WCM253_Z1_1 | 13 | 19600515 | 30857822 | −0.2317 | 0.4834 | 1.8396 | 0.9564 | 2 | 1 |
| WCM253_Z1_1 | 13 | 31035629 | 60348886 | −0.9813 | −0.8627 | 1.0379 | 0.0619 | 1 | 0 |
| WCM253_Z1_1 | 13 | 60384944 | 115089411 | −0.1869 | 0.5499 | 1.9411 | 0.9868 | 2 | 1 |
| WCM253_Z1_1 | 14 | 19377716 | 31097471 | 0.6097 | 1.6112 | 4.1463 | 1.9638 | 4 | 2 |
| WCM253_Z1_1 | 14 | 31099738 | 107283203 | 0.523 | 1.5041 | 4.6903 | 0.9825 | 5 | 1 |
| WCM253_Z1_1 | 15 | 20170122 | 35275599 | −0.1773 | 0.564 | 1.8131 | 1.1437 | 2 | 1 |
| WCM253_Z1_1 | 15 | 35664399 | 47873549 | −0.504 | 0.0539 | 2.1281 | −0.052 | 2 | 0 |
| WCM253_Z1_1 | 15 | 48052073 | 70961375 | −0.1802 | 0.5598 | 1.9274 | 1.0207 | 2 | 1 |
| WCM253_Z1_1 | 15 | 70961689 | 81282019 | −0.4989 | 0.0623 | 1.0737 | 1.0145 | 1 | 1 |
| WCM253_Z1_1 | 15 | 81289814 | 88727457 | −0.2106 | 0.5149 | 1.9216 | 0.9361 | 2 | 1 |
| WCM253_Z1_1 | 15 | 88799174 | 90016029 | −0.643 | −0.1864 | 0.9339 | 0.8237 | 1 | 1 |
| WCM253_Z1_1 | 15 | 90020057 | 102506276 | −0.1465 | 0.6091 | 2.0426 | 1.008 | 2 | 1 |
| WCM253_Z1_1 | 16 | 97541 | 20043506 | 0.1467 | 1.0184 | 2.2133 | 1.8379 | 2 | 2 |
| WCM253_Z1_1 | 16 | 20043735 | 49433056 | −0.1588 | 0.5912 | 2.0744 | 0.9385 | 2 | 1 |
| WCM253_Z1_1 | 16 | 49525190 | 70765429 | −0.4584 | 0.1291 | 2.1757 | 0.0116 | 2 | 0 |
| WCM253_Z1_1 | 16 | 70778474 | 74339202 | 0.145 | 1.0161 | 2.1257 | 1.9192 | 2 | 2 |
| WCM253_Z1_1 | 16 | 74339594 | 90233622 | −0.5194 | 0.028 | 2.0654 | −0.0263 | 2 | 0 |
| WCM253_Z1_1 | 17 | 131595 | 16285747 | −0.5128 | 0.0392 | 2.0575 | −0.0025 | 2 | 0 |
| WCM253_Z1_1 | 17 | 16321061 | 16344386 | 0.2253 | 1.123 | NA | NA | NA | NA |
| WCM253_Z1_1 | 17 | 16344726 | 19648426 | 0.1812 | 1.0645 | 2.1834 | 1.9995 | 2 | 2 |
| WCM253_Z1_1 | 17 | 19651682 | 26488217 | −0.5117 | 0.0409 | 1.5907 | 0.4669 | 2 | 0 |
| WCM253_Z1_1 | 17 | 26490633 | 37341052 | 0.1624 | 1.0395 | 2.0995 | 2.0115 | 2 | 2 |
| WCM253_Z1_1 | 17 | 37341385 | 44827171 | 0.4232 | 1.3788 | 3.0783 | 2.1227 | 3 | 2 |
| WCM253_Z1_1 | 17 | 44828970 | 75398418 | 0.154 | 1.0282 | 3.0457 | 1.0332 | 3 | 1 |
| WCM253_Z1_1 | 17 | 75398774 | 80159616 | −0.0334 | 0.7709 | 2.4556 | 0.9572 | 2 | 1 |
| WCM253_Z1_1 | 17 | 80159669 | 80320400 | 0.2077 | 1.0998 | NA | NA | NA | NA |
| WCM253_Z1_1 | 17 | 80332251 | 81083567 | 0.8326 | 1.8803 | 6.4038 | 0.9594 | 6 | 1 |
| WCM253_Z1_1 | 18 | 48236 | 53696076 | 0.0487 | 0.8852 | 1.8725 | 1.8216 | 2 | 2 |
| WCM253_Z1_1 | 18 | 53723847 | 67992210 | 0.328 | 1.257 | 2.5288 | 2.2511 | 3 | 2 |
| WCM253_Z1_1 | 18 | 67992484 | 70532807 | −0.0451 | 0.7544 | NA | NA | NA | NA |
| WCM253_Z1_1 | 18 | 71740779 | 78005226 | −0.7282 | −0.3426 | 1.5274 | 0.0498 | 2 | 0 |
| WCM253_Z1_1 | 19 | 281435 | 3805998 | −0.1333 | 0.6283 | 2.1127 | 0.9789 | 2 | 1 |
| WCM253_Z1_1 | 19 | 3808876 | 16687318 | −0.5369 | −0.0015 | 1.1649 | 0.8329 | 1 | 1 |
| WCM253_Z1_1 | 19 | 16687574 | 17845156 | 0.6428 | 1.6517 | 5.1221 | 1.1622 | 5 | 1 |
| WCM253_Z1_1 | 19 | 17865979 | 18885764 | 0.0417 | 0.8755 | 2.3811 | 1.2883 | 2 | 1 |
| WCM253_Z1_1 | 19 | 18886579 | 43568748 | −0.1659 | 0.5808 | 1.9535 | 1.0378 | 2 | 1 |
| WCM253_Z1_1 | 19 | 43570713 | 50391765 | 0.2197 | 1.1156 | 3.2113 | 1.1223 | 3 | 1 |
| WCM253_Z1_1 | 19 | 50393182 | 57839567 | 0.0282 | 0.8569 | 2.6256 | 0.9966 | 3 | 1 |
| WCM253_Z1_1 | 19 | 57839678 | 59083923 | 0.2112 | 1.1043 | 3.0632 | 1.2368 | 3 | 1 |
| WCM253_Z1_1 | 2 | 41609 | 97560812 | 0.1154 | 0.9761 | 1.9911 | 1.9432 | 2 | 2 |
| WCM253_Z1_1 | 2 | 97568441 | 144525573 | −0.2021 | 0.5275 | 1.9584 | 0.9244 | 2 | 1 |
| WCM253_Z1_1 | 2 | 144704631 | 148674856 | 0.1275 | 0.9926 | NA | NA | NA | NA |
| WCM253_Z1_1 | 2 | 148676071 | 170367093 | 0.4216 | 1.3767 | 2.6888 | 2.5045 | 3 | 3 |
| WCM253_Z1_1 | 2 | 170367356 | 206608039 | 0.0386 | 0.8713 | 2.8364 | 0.8221 | 3 | 1 |
| WCM253_Z1_1 | 2 | 206608278 | 242946607 | −0.2273 | 0.49 | 2.8236 | −0.0147 | 3 | 0 |
| WCM253_Z1_1 | 20 | 68373 | 419927 | −0.4851 | 0.0852 | 1.1697 | 0.952 | 1 | 1 |
| WCM253_Z1_1 | 20 | 420894 | 1963643 | −1.0747 | −1.0837 | 0.8872 | 0.0565 | 1 | 0 |
| WCM253_Z1_1 | 20 | 2082677 | 20349226 | −0.486 | 0.0838 | 1.0879 | 1.0317 | 1 | 1 |
| WCM253_Z1_1 | 20 | 20349746 | 30602830 | −0.1685 | 0.577 | 1.9859 | 0.9976 | 2 | 1 |
| WCM253_Z1_1 | 20 | 30605642 | 31873994 | −0.5712 | −0.0602 | 0.9955 | 0.9227 | 1 | 1 |
| WCM253_Z1_1 | 20 | 31876576 | 33867903 | −0.0192 | 0.7908 | 2.1836 | 1.2766 | 2 | 1 |
| WCM253_Z1_1 | 20 | 33868618 | 35632100 | 0.4674 | 1.4345 | NA | NA | NA | NA |
| WCM253_Z1_1 | 20 | 35635934 | 44351065 | −0.1084 | 0.6642 | 1.6531 | 1.5163 | 2 | 2 |
| WCM253_Z1_1 | 20 | 44352620 | 45138254 | 0.3905 | 1.3371 | 3.8021 | 1.2508 | 4 | 1 |
| WCM253_Z1_1 | 20 | 45140720 | 48744705 | −0.3678 | 0.2746 | 1.3715 | 1.0478 | 1 | 1 |
| WCM253_Z1_1 | 20 | 48746096 | 50287790 | 0.4378 | 1.3972 | NA | NA | NA | NA |
| WCM253_Z1_1 | 20 | 50290707 | 62120458 | −0.1564 | 0.5947 | 1.9577 | 1.0625 | 2 | 1 |
| WCM253_Z1_1 | 20 | 62121784 | 62904845 | 0.6337 | 1.6407 | NA | NA | NA | NA |
| WCM253_Z1_1 | 21 | 9826207 | 40182005 | 0.081 | 0.9295 | 2.8612 | 0.948 | 3 | 1 |
| WCM253_Z1_1 | 21 | 40184934 | 42866410 | −0.3309 | 0.3322 | 1.3976 | 1.1202 | 1 | 1 |
| WCM253_Z1_1 | 21 | 42870050 | 48084277 | 0.0534 | 0.8917 | 2.7326 | 0.9781 | 3 | 1 |
| WCM253_Z1_1 | 22 | 17063476 | 42981262 | −0.1354 | 0.6252 | 2.0145 | 1.0703 | 2 | 1 |
| WCM253_Z1_1 | 22 | 42981890 | 51159796 | −0.5135 | 0.038 | 1.0611 | 0.9923 | 1 | 1 |
| WCM253_Z1_1 | 22 | 51159919 | 51215138 | −0.2163 | 0.5064 | NA | NA | NA | NA |
| WCM253_Z1_1 | 3 | 239459 | 43760010 | −0.1777 | 0.5635 | 1.9385 | 1.0173 | 2 | 1 |
| WCM253_Z1_1 | 3 | 44155784 | 78706315 | −0.5007 | 0.0594 | 1.0715 | 1.0125 | 1 | 1 |
| WCM253_Z1_1 | 3 | 78708941 | 93755468 | −0.2869 | 0.3999 | NA | NA | NA | NA |
| WCM253_Z1_1 | 3 | 93758825 | 96706450 | 0.3868 | 1.3325 | NA | NA | NA | NA |
| WCM253_Z1_1 | 3 | 96706710 | 109178758 | 0.0492 | 0.8858 | 1.9826 | 1.7131 | 2 | 2 |
| WCM253_Z1_1 | 3 | 109195587 | 132390687 | 0.3928 | 1.3401 | 2.893 | 2.1702 | 3 | 2 |
| WCM253_Z1_1 | 3 | 132390911 | 168542644 | 0.1379 | 1.0065 | 2.0766 | 1.9414 | 2 | 2 |
| WCM253_Z1_1 | 3 | 168546991 | 190578594 | 0.4257 | 1.3819 | 2.8835 | 2.3289 | 3 | 2 |
| WCM253_Z1_1 | 3 | 190930341 | 197894712 | −0.0552 | 0.7401 | 2.0937 | 1.2469 | 2 | 1 |
| WCM253_Z1_1 | 4 | 59345 | 3040147 | −0.5352 | 0.0013 | 1.1756 | 0.8262 | 1 | 1 |
| WCM253_Z1_1 | 4 | 3042322 | 37633055 | −0.8849 | −0.6531 | 0.9393 | 0.3325 | 1 | 0 |
| WCM253_Z1_1 | 4 | 37636680 | 38829989 | −0.2997 | 0.3803 | NA | NA | NA | NA |
| WCM253_Z1_1 | 4 | 38830234 | 155749212 | −0.552 | −0.0272 | 1.1272 | 0.8355 | 1 | 1 |
| WCM253_Z1_1 | 4 | 156135228 | 190426570 | −0.2257 | 0.4923 | 1.8417 | 0.9716 | 2 | 1 |
| WCM253_Z1_1 | 5 | 140532 | 56170944 | −0.0581 | 0.7361 | 2.2156 | 1.1156 | 2 | 1 |
| WCM253_Z1_1 | 5 | 56171126 | 125696034 | −0.4695 | 0.1109 | 1.2112 | 0.9486 | 1 | 1 |
| WCM253_Z1_1 | 5 | 125707167 | 180687812 | −0.1981 | 0.5334 | 1.8951 | 0.9996 | 2 | 1 |
| WCM253_Z1_1 | 6 | 203624 | 3751682 | −0.538 | −0.0034 | 1.7406 | 0.2546 | 2 | 0 |
| WCM253_Z1_1 | 6 | 3850216 | 7585995 | 0.3164 | 1.242 | 2.7999 | 1.9308 | 3 | 2 |
| WCM253_Z1_1 | 6 | 7590624 | 18130933 | −0.2812 | 0.4086 | 1.5923 | 1.0625 | 2 | 1 |
| WCM253_Z1_1 | 6 | 18132389 | 47846965 | 0.07 | 0.9144 | 1.9209 | 1.8487 | 2 | 2 |
| WCM253_Z1_1 | 6 | 47847262 | 57244780 | −0.5177 | 0.0308 | 2.0601 | −0.0169 | 2 | 0 |
| WCM253_Z1_1 | 6 | 57246884 | 72006097 | −0.9151 | −0.7171 | 0.8624 | 0.3542 | 1 | 0 |
| WCM253_Z1_1 | 6 | 72006512 | 136904711 | −0.537 | −0.0018 | 2.0048 | −0.0073 | 2 | 0 |
| WCM253_Z1_1 | 6 | 136913416 | 160222208 | −0.2431 | 0.4663 | 2.7375 | 0.0256 | 3 | 0 |
| WCM253_Z1_1 | 6 | 160224313 | 170893408 | −0.5464 | −0.0176 | 1.9588 | 0.0169 | 2 | 0 |
| WCM253_Z1_1 | 7 | 193253 | 331348 | −0.12 | 0.6475 | NA | NA | NA | NA |
| WCM253_Z1_1 | 7 | 539958 | 30665896 | 0.0549 | 0.8937 | 1.8885 | 1.8272 | 2 | 2 |
| WCM253_Z1_1 | 7 | 30668260 | 43531763 | −0.2352 | 0.4782 | 1.7834 | 1.0025 | 2 | 1 |
| WCM253_Z1_1 | 7 | 43532712 | 64437406 | 0.0437 | 0.8783 | 1.8991 | 1.7775 | 2 | 2 |
| WCM253_Z1_1 | 7 | 64437584 | 103808935 | 0.0744 | 0.9204 | 2.8189 | 0.9663 | 3 | 1 |
| WCM253_Z1_1 | 7 | 103824464 | 104731771 | 0.0245 | 0.8518 | NA | NA | NA | NA |
| WCM253_Z1_1 | 7 | 104742021 | 158937400 | −0.2323 | 0.4825 | 1.8365 | 0.9578 | 2 | 1 |
| WCM253_Z1_1 | 8 | 190897 | 12809667 | −0.2698 | 0.426 | 1.7695 | 0.9174 | 2 | 1 |
| WCM253_Z1_1 | 8 | 12848399 | 42761326 | −0.556 | −0.034 | 1.9111 | 0.0423 | 2 | 0 |
| WCM253_Z1_1 | 8 | 42780709 | 49987799 | 0.3867 | 1.3323 | NA | NA | NA | NA |
| WCM253_Z1_1 | 8 | 50449716 | 55638922 | 0.8228 | 1.8687 | 5.8648 | 1.4395 | 6 | 1 |
| WCM253_Z1_1 | 8 | 55647242 | 69243602 | 0.2471 | 1.1517 | 2.8488 | 1.5946 | 3 | 2 |
| WCM253_Z1_1 | 8 | 69351868 | 82751929 | 0.4813 | 1.452 | 3.9161 | 1.5557 | 4 | 2 |
| WCM253_Z1_1 | 8 | 82752126 | 134267581 | 0.2505 | 1.1561 | 2.7781 | 1.6789 | 3 | 2 |
| WCM253_Z1_1 | 8 | 134269104 | 146279472 | −0.2017 | 0.528 | 2.8864 | −0.0026 | 3 | 0 |
| WCM253_Z1_1 | 9 | 17056 | 1057249 | 0.0068 | 0.8272 | 2.0335 | 1.515 | 2 | 2 |
| WCM253_Z1_1 | 9 | 2029140 | 19446231 | 0.2304 | 1.1298 | 2.566 | 1.8106 | 3 | 2 |
| WCM253_Z1_1 | 9 | 19450449 | 79326097 | 0.0085 | 0.8295 | 1.9724 | 1.5818 | 2 | 2 |
| WCM253_Z1_1 | 9 | 79326270 | 82337885 | −0.3267 | 0.3387 | NA | NA | NA | NA |
| WCM253_Z1_1 | 9 | 82339978 | 98242669 | −0.8091 | −0.4987 | 1.4315 | −0.016 | 1 | 0 |
| WCM253_Z1_1 | 9 | 98244421 | 98783187 | −0.5029 | 0.0557 | NA | NA | NA | NA |
| WCM253_Z1_1 | 9 | 98783369 | 133947012 | −0.3076 | 0.3682 | 2.5874 | −0.0059 | 3 | 0 |
| WCM253_Z1_1 | 9 | 133948049 | 141111414 | −0.1929 | 0.541 | 3.0266 | −0.1166 | 3 | 0 |
| WCM253_Z1_1 | X | 2700163 | 63410922 | −0.0175 | 0.7932 | NA | NA | NA | NA |
| WCM253_Z1_1 | X | 63411160 | 145109356 | −0.557 | −0.0357 | NA | NA | NA | NA |
| WCM253_Z1_1 | X | 147800748 | 154509348 | 0.0849 | 0.9348 | NA | NA | NA | NA |
| WCM261 | 1 | 14860 | 249213973 | 0.0349 | NA | NA | NA | NA | NA |
| WCM261 | 10 | 226106 | 135477898 | 0.0235 | NA | NA | NA | NA | NA |
| WCM261 | 11 | 193146 | 134605849 | 0.0169 | NA | NA | NA | NA | NA |
| WCM261 | 12 | 148388 | 133810892 | 0.0029 | NA | NA | NA | NA | NA |
| WCM261 | 13 | 19041979 | 115089411 | −0.0069 | NA | NA | NA | NA | NA |
| WCM261 | 14 | 19110297 | 107283269 | 0.014 | NA | NA | NA | NA | NA |
| WCM261 | 15 | 20083887 | 102515229 | 0.0363 | NA | NA | NA | NA | NA |
| WCM261 | 16 | 83338 | 90260461 | 0.0065 | NA | NA | NA | NA | NA |
| WCM261 | 17 | 131595 | 81187849 | 0.026 | NA | NA | NA | NA | NA |
| WCM261 | 18 | 47686 | 78005145 | −0.004 | NA | NA | NA | NA | NA |
| WCM261 | 19 | 105010 | 59093696 | −0.0067 | NA | NA | NA | NA | NA |
| WCM261 | 2 | 41662 | 243037019 | 0.0094 | NA | NA | NA | NA | NA |
| WCM261 | 20 | 68337 | 62934623 | 0.0462 | NA | NA | NA | NA | NA |
| WCM261 | 21 | 9590331 | 48084201 | 0.0142 | NA | NA | NA | NA | NA |
| WCM261 | 22 | 16159046 | 51216331 | 0.0524 | NA | NA | NA | NA | NA |
| WCM261 | 3 | 239431 | 197896700 | 0.0189 | NA | NA | NA | NA | NA |
| WCM261 | 4 | 53377 | 190903980 | 0.0088 | NA | NA | NA | NA | NA |
| WCM261 | 5 | 90576 | 180687576 | 0.0022 | NA | NA | NA | NA | NA |
| WCM261 | 6 | 203516 | 170892848 | 0.008 | NA | NA | NA | NA | NA |
| WCM261 | 7 | 195656 | 158937013 | −5.00E−04 | NA | NA | NA | NA | NA |
| WCM261 | 8 | 163470 | 146279529 | 0.0056 | NA | NA | NA | NA | NA |
| WCM261 | 9 | 14908 | 141107582 | 0.0292 | NA | NA | NA | NA | NA |
| WCM261 | X | 2700139 | 154493406 | −0.1254 | NA | NA | NA | NA | NA |
| WCM261_Z4_1 | 1 | 69640 | 145112428 | −0.3656 | 0.1398 | 1.1263 | 1.0772 | 1 | 1 |
| WCM261_Z4_1 | 1 | 145115810 | 190424021 | 0.1862 | 1.0978 | 3.2188 | 1.0619 | 3 | 1 |
| WCM261_Z4_1 | 1 | 192128396 | 193121560 | −0.8586 | −1.1315 | NA | NA | NA | NA |
| WCM261_Z4_1 | 1 | 193149667 | 249212550 | 0.185 | 1.096 | 3.1726 | 1.1025 | 3 | 1 |
| WCM261_Z4_1 | 10 | 225958 | 135440086 | −0.3527 | 0.1657 | 2.2014 | 0.042 | 2 | 0 |
| WCM261_Z4_1 | 11 | 193112 | 2161519 | −0.3226 | 0.2254 | 1.6514 | 0.6868 | 2 | 1 |
| WCM261_Z4_1 | 11 | 2161950 | 102496036 | −0.0504 | 0.7189 | 2.1841 | 1.1079 | 2 | 1 |
| WCM261_Z4_1 | 11 | 102554079 | 110697305 | −0.324 | 0.2227 | 1.6648 | 0.669 | 2 | 1 |
| WCM261_Z4_1 | 11 | 111152166 | 134856626 | 0.1646 | 1.0648 | 2.1208 | 2.0629 | 2 | 2 |
| WCM261_Z4_1 | 12 | 148673 | 122626860 | 0.1714 | 1.0753 | 2.133 | 2.0813 | 2 | 2 |
| WCM261_Z4_1 | 12 | 122657189 | 133810703 | −0.339 | 0.193 | 2.1423 | 0.144 | 2 | 0 |
| WCM261_Z4_1 | 13 | 19600365 | 32313777 | 0.3689 | 1.3683 | 4.1581 | 1.0052 | 4 | 1 |
| WCM261_Z4_1 | 13 | 32332471 | 39265108 | 0.0721 | 0.9199 | 2.9398 | 0.8442 | 3 | 1 |
| WCM261_Z4_1 | 13 | 39265305 | 39541159 | 0.346 | 1.3353 | NA | NA | NA | NA |
| WCM261_Z4_1 | 13 | 39542673 | 90883435 | −0.7321 | −0.7333 | 1.1497 | 0.0534 | 1 | 0 |
| WCM261_Z4_1 | 13 | 92003159 | 115089411 | 0.1684 | 1.0706 | 3.1591 | 1.0415 | 3 | 1 |
| WCM261_Z4_1 | 14 | 19377703 | 57099859 | −0.0551 | 0.7111 | 2.1541 | 1.1201 | 2 | 1 |
| WCM261_Z4_1 | 14 | 57101660 | 82000024 | −0.3627 | 0.1456 | 1.1397 | 1.0727 | 1 | 1 |
| WCM261_Z4_1 | 14 | 85995090 | 107283094 | −0.069 | 0.6875 | 2.1308 | 1.0903 | 2 | 1 |
| WCM261_Z4_1 | 15 | 20170134 | 41988440 | −0.734 | −0.7386 | 1.1796 | 0.0191 | 1 | 0 |
| WCM261_Z4_1 | 15 | 41988711 | 102517662 | −0.0805 | 0.6679 | 2.1158 | 1.0618 | 2 | 1 |
| WCM261_Z4_1 | 16 | 97540 | 46615932 | −0.0576 | 0.7069 | 2.1788 | 1.0859 | 2 | 1 |
| WCM261_Z4_1 | 16 | 46617439 | 71884775 | −0.371 | 0.1287 | 1.14 | 1.0467 | 1 | 1 |
| WCM261_Z4_1 | 16 | 71884997 | 90233626 | −0.7056 | −0.6582 | 1.1903 | 0.0771 | 1 | 0 |
| WCM261_Z4_1 | 17 | 131595 | 81083567 | −0.0669 | 0.6912 | 2.1219 | 1.1073 | 2 | 1 |
| WCM261_Z4_1 | 18 | 48201 | 78005214 | −0.3592 | 0.1526 | 2.1416 | 0.0815 | 2 | 0 |
| WCM261_Z4_1 | 19 | 281506 | 21992341 | −0.0677 | 0.6898 | 2.1557 | 1.0704 | 2 | 1 |
| WCM261_Z4_1 | 19 | 21992542 | 48176995 | −0.36 | 0.151 | 1.3274 | 0.8932 | 1 | 1 |
| WCM261_Z4_1 | 19 | 48179138 | 59083953 | 0.1303 | 1.0116 | 2.9624 | 1.0699 | 3 | 1 |
| WCM261_Z4_1 | 2 | 41613 | 47797392 | 0.1571 | 1.0532 | 3.0905 | 1.0599 | 3 | 1 |
| WCM261_Z4_1 | 2 | 47905398 | 70528091 | −0.1267 | 0.588 | 3.0238 | −0.0175 | 3 | 0 |
| WCM261_Z4_1 | 2 | 70528601 | 106015995 | −0.0913 | 0.6495 | 2.1537 | 0.9835 | 2 | 1 |
| WCM261_Z4_1 | 2 | 106471734 | 115991546 | −0.4138 | 0.0404 | 1.9598 | 0.097 | 2 | 0 |
| WCM261_Z4_1 | 2 | 116066907 | 125669051 | −0.0608 | 0.7014 | 1.9098 | 1.3425 | 2 | 1 |
| WCM261_Z4_1 | 2 | 125671800 | 157369859 | −0.4031 | 0.0629 | 2.0763 | 0.0127 | 2 | 0 |
| WCM261_Z4_1 | 2 | 157406249 | 242946607 | −0.1162 | 0.6064 | 2.0101 | 1.0348 | 2 | 1 |
| WCM261_Z4_1 | 20 | 68351 | 397883 | 0.0676 | 0.9126 | NA | NA | NA | NA |
| WCM261_Z4_1 | 20 | 398171 | 25666211 | 0.2061 | 1.1281 | 2.2155 | 2.1559 | 2 | 2 |
| WCM261_Z4_1 | 20 | 25666642 | 62904845 | −0.0462 | 0.7261 | 2.1605 | 1.1479 | 2 | 1 |
| WCM261_Z4_1 | 21 | 9826203 | 48084277 | 0.1601 | 1.0578 | 2.1362 | 2.0271 | 2 | 2 |
| WCM261_Z4_1 | 22 | 16287449 | 51215124 | −0.0639 | 0.6962 | 2.1589 | 1.0817 | 2 | 1 |
| WCM261_Z4_1 | 3 | 239349 | 17550038 | 0.1808 | 1.0896 | 3.2448 | 1.0114 | 3 | 1 |
| WCM261_Z4_1 | 3 | 18055231 | 23258777 | −0.4257 | 0.0155 | NA | NA | NA | NA |
| WCM261_Z4_1 | 3 | 23312442 | 74316427 | 0.2051 | 1.1267 | 3.22 | 1.1471 | 3 | 1 |
| WCM261_Z4_1 | 3 | 74334621 | 197894719 | 0.3827 | 1.388 | 3.1677 | 2.0667 | 3 | 2 |
| WCM261_Z4_1 | 4 | 59345 | 111542124 | −0.0529 | 0.7148 | 2.1314 | 1.1512 | 2 | 1 |
| WCM261_Z4_1 | 4 | 111542366 | 133367616 | −0.3854 | 0.0993 | 2.1773 | −0.0348 | 2 | 0 |
| WCM261_Z4_1 | 4 | 133368196 | 138442573 | −1.3657 | NA | NA | NA | NA | NA |
| WCM261_Z4_1 | 4 | 138442795 | 138453409 | −0.3506 | 0.1699 | NA | NA | NA | NA |
| WCM261_Z4_1 | 4 | 139100391 | 190426502 | −0.0333 | 0.7477 | 2.2095 | 1.1486 | 2 | 1 |
| WCM261_Z4_1 | 5 | 140532 | 31294164 | −0.0421 | 0.7329 | 2.1976 | 1.1264 | 2 | 1 |
| WCM261_Z4_1 | 5 | 31294357 | 49699122 | 0.1863 | 1.0981 | 3.0231 | 1.2582 | 3 | 1 |
| WCM261_Z4_1 | 5 | 49706767 | 53606301 | −0.0963 | 0.6408 | NA | NA | NA | NA |
| WCM261_Z4_1 | 5 | 53751868 | 90672527 | −0.3797 | 0.1111 | 1.1264 | 1.0337 | 1 | 1 |
| WCM261_Z4_1 | 5 | 90678654 | 112927740 | −0.6583 | −0.5304 | 1.1947 | 0.19 | 1 | 0 |
| WCM261_Z4_1 | 5 | 113698581 | 118850687 | −0.2379 | 0.3874 | 1.4371 | 1.179 | 1 | 1 |
| WCM261_Z4_1 | 5 | 118860935 | 180687766 | 0.19 | 1.1037 | 3.2008 | 1.0972 | 3 | 1 |
| WCM261_Z4_1 | 6 | 203509 | 22570189 | −0.3321 | 0.2068 | 1.1929 | 1.1153 | 1 | 1 |
| WCM261_Z4_1 | 6 | 22570464 | 68599049 | −0.0722 | 0.6822 | 2.0854 | 1.1237 | 2 | 1 |
| WCM261_Z4_1 | 6 | 69348819 | 69945012 | −0.457 | −0.0517 | NA | NA | NA | NA |
| WCM261_Z4_1 | 6 | 69949086 | 102516268 | −0.7447 | −0.7697 | 1.1488 | 0.0243 | 1 | 0 |
| WCM261_Z4_1 | 6 | 105177609 | 126332504 | −0.3639 | 0.1432 | 2.1288 | 0.0799 | 2 | 0 |
| WCM261_Z4_1 | 6 | 126334135 | 170893406 | −0.0792 | 0.6701 | 2.1078 | 1.0746 | 2 | 1 |
| WCM261_Z4_1 | 7 | 193205 | 8043669 | −0.0386 | 0.7389 | 2.2813 | 1.0564 | 2 | 1 |
| WCM261_Z4_1 | 7 | 8062198 | 57529304 | 0.2298 | 1.164 | 2.2845 | 2.197 | 2 | 2 |
| WCM261_Z4_1 | 7 | 57529644 | 110764849 | 0.3862 | 1.393 | 3.148 | 2.1044 | 3 | 2 |
| WCM261_Z4_1 | 7 | 111032062 | 113558694 | 0.1756 | 1.0817 | NA | NA | NA | NA |
| WCM261_Z4_1 | 7 | 114066583 | 115624478 | −1.3012 | −4.7226 | NA | NA | NA | NA |
| WCM261_Z4_1 | 7 | 115750875 | 154002623 | 0.205 | 1.1265 | 2.2297 | 2.137 | 2 | 2 |
| WCM261_Z4_1 | 7 | 154143361 | 156786875 | −0.2749 | 0.3178 | 2.2649 | 0.2279 | 2 | 0 |
| WCM261_Z4_1 | 7 | 156798302 | 158937203 | 0.2307 | 1.1653 | NA | NA | NA | NA |
| WCM261_Z4_1 | 8 | 190907 | 8239230 | 0.5603 | 1.6359 | 3.29 | 2.9257 | 3 | 3 |
| WCM261_Z4_1 | 8 | 8559908 | 32621591 | −0.0745 | 0.6782 | 3.1687 | 0.0317 | 3 | 0 |
| WCM261_Z4_1 | 8 | 33230244 | 57905962 | 0.5825 | 1.666 | 3.2336 | 3.1132 | 3 | 3 |
| WCM261_Z4_1 | 8 | 59058907 | 65286010 | 0.1501 | 1.0424 | NA | NA | NA | NA |
| WCM261_Z4_1 | 8 | 65290081 | 82752149 | 0.5804 | 1.6633 | 3.4079 | 2.9268 | 3 | 3 |
| WCM261_Z4_1 | 8 | 85097244 | 87497188 | −0.084 | 0.662 | NA | NA | NA | NA |
| WCM261_Z4_1 | 8 | 87498764 | 113812467 | 0.5501 | 1.6219 | 3.2629 | 2.8928 | 3 | 3 |
| WCM261_Z4_1 | 8 | 113841912 | 118184872 | −0.0506 | 0.7188 | NA | NA | NA | NA |
| WCM261_Z4_1 | 8 | 118533170 | 146279489 | 0.606 | 1.6979 | 3.2442 | 3.2442 | 3 | 3 |
| WCM261_Z4_1 | 9 | 17074 | 141111414 | −0.0593 | 0.7041 | 2.1694 | 1.0888 | 2 | 1 |
| WCM261_Z4_1 | X | 2700147 | 64141841 | −0.3873 | 0.0954 | NA | NA | NA | NA |
| WCM261_Z4_1 | X | 64196252 | 68049691 | 2.4189 | 3.8311 | NA | NA | NA | NA |
| WCM261_Z4_1 | X | 68058523 | 109419611 | 0.1646 | 1.0647 | NA | NA | NA | NA |
| WCM261_Z4_1 | X | 109441769 | 109698481 | −0.9619 | −1.5272 | NA | NA | NA | NA |
| WCM261_Z4_1 | X | 109919491 | 154509348 | 0.1631 | 1.0624 | NA | NA | NA | NA |
| WCM2_Z3_1 | 1 | 69592 | 109200198 | −0.162 | −0.0117 | 1.0128 | 0.9711 | 1 | 1 |
| WCM2_Z3_1 | 1 | 109202492 | 110527726 | −0.4807 | −0.909 | 1.1142 | −0.049 | 1 | 0 |
| WCM2_Z3_1 | 1 | 110551727 | 205767885 | −0.1618 | −0.0112 | 1.0177 | 0.9668 | 1 | 1 |
| WCM2_Z3_1 | 1 | 205768191 | 216172264 | −0.5283 | −1.0821 | 1.0423 | −0.0976 | 1 | 0 |
| WCM2_Z3_1 | 1 | 216173794 | 236208773 | −0.1864 | −0.0692 | 1.8646 | 0.0417 | 2 | 0 |
| WCM2_Z3_1 | 1 | 236212176 | 249212557 | 0.0352 | 0.413 | 1.5945 | 1.0684 | 2 | 1 |
| WCM2_Z3_1 | 10 | 225988 | 293361 | −0.2338 | −0.1853 | NA | NA | NA | NA |
| WCM2_Z3_1 | 10 | 294457 | 82012599 | 0.0613 | 0.4646 | 1.913 | 0.847 | 2 | 1 |
| WCM2_Z3_1 | 10 | 82013146 | 88466450 | −0.108 | 0.1114 | 2.122 | 0.0385 | 2 | 0 |
| WCM2_Z3_1 | 10 | 88469733 | 135440108 | −0.258 | −0.2469 | 0.8796 | 0.8058 | 1 | 1 |
| WCM2_Z3_1 | 11 | 193112 | 533360 | 0.2277 | 0.7747 | 2.567 | 0.8545 | 3 | 1 |
| WCM2_Z3_1 | 11 | 533611 | 34654150 | 0.1449 | 0.6242 | 2.711 | 0.3716 | 3 | 0 |
| WCM2_Z3_1 | 11 | 34668175 | 134856523 | 0.0398 | 0.4221 | 1.8517 | 0.8281 | 2 | 1 |
| WCM2_Z3_1 | 12 | 148674 | 9847465 | 0.1212 | 0.5797 | 1.9401 | 1.0489 | 2 | 1 |
| WCM2_Z3_1 | 12 | 9871328 | 32137512 | −0.213 | −0.1338 | 1.8398 | −0.0169 | 2 | 0 |
| WCM2_Z3_1 | 12 | 32137682 | 125263024 | 0.0967 | 0.5331 | 1.9905 | 0.9036 | 2 | 1 |
| WCM2_Z3_1 | 12 | 125267352 | 133810703 | −0.1952 | −0.0905 | 1.8384 | 0.0401 | 2 | 0 |
| WCM2_Z3_1 | 13 | 19600475 | 115089411 | −0.2179 | −0.1457 | 1.8515 | −0.0436 | 2 | 0 |
| WCM2_Z3_1 | 14 | 19377703 | 59113147 | 0.2454 | 0.8058 | 2.1323 | 1.3639 | 2 | 1 |
| WCM2_Z3_1 | 14 | 59113430 | 74439698 | −0.1825 | −0.06 | 1.9372 | −0.0187 | 2 | 0 |
| WCM2_Z3_1 | 14 | 74440597 | 86089622 | 0.1144 | 0.5669 | 1.7508 | 1.2119 | 2 | 1 |
| WCM2_Z3_1 | 14 | 86089790 | 95670564 | 0.2789 | 0.8642 | 2.8615 | 0.779 | 3 | 1 |
| WCM2_Z3_1 | 14 | 95670669 | 95875449 | −0.0036 | 0.3344 | NA | NA | NA | NA |
| WCM2_Z3_1 | 14 | 95875689 | 105964244 | 0.5136 | 1.2486 | 2.824 | 1.9282 | 3 | 2 |
| WCM2_Z3_1 | 14 | 105965102 | 107283080 | 0.4259 | 1.1096 | NA | NA | NA | NA |
| WCM2_Z3_1 | 15 | 20169912 | 102506276 | −0.1957 | −0.0917 | 0.9633 | 0.9135 | 1 | 1 |
| WCM2_Z3_1 | 16 | 66930 | 47007464 | 0.3853 | 1.0435 | 2.0782 | 2.0443 | 2 | 2 |
| WCM2_Z3_1 | 16 | 47117174 | 90233626 | −0.169 | −0.0281 | 1.954 | 0.0075 | 2 | 0 |
| WCM2_Z3_1 | 17 | 131595 | 3324660 | −0.5106 | −1.0161 | 0.9295 | 0.0595 | 1 | 0 |
| WCM2_Z3_1 | 17 | 3324810 | 3979949 | 0.1305 | 0.5972 | 2.035 | 0.9905 | 2 | 1 |
| WCM2_Z3_1 | 17 | 3980179 | 4016096 | 0.0374 | 0.4173 | NA | NA | NA | NA |
| WCM2_Z3_1 | 17 | 4017750 | 6330367 | 0.2149 | 0.7518 | 3.3842 | −0.0165 | 3 | 0 |
| WCM2_Z3_1 | 17 | 6331822 | 10618855 | −0.1619 | −0.0113 | 2.0746 | −0.0902 | 2 | 0 |
| WCM2_Z3_1 | 17 | 10628422 | 11532835 | −0.4752 | −0.8897 | NA | NA | NA | NA |
| WCM2_Z3_1 | 17 | 11535977 | 17398632 | −0.5832 | −1.3034 | 0.708 | 0.1023 | 1 | 0 |
| WCM2_Z3_1 | 17 | 17398840 | 81083567 | −0.1577 | −0.0017 | 1.1755 | 0.8222 | 1 | 1 |
| WCM2_Z3_1 | 18 | 48269 | 78005215 | −0.1607 | −0.0087 | 1.9125 | 0.0754 | 2 | 0 |
| WCM2_Z3_1 | 19 | 281508 | 40366362 | −0.1469 | 0.0234 | 2.0445 | −0.0118 | 2 | 0 |
| WCM2_Z3_1 | 19 | 40367908 | 40373952 | −1.4168 | NA | NA | NA | NA | NA |
| WCM2_Z3_1 | 19 | 40376653 | 59083953 | −0.1233 | 0.0772 | 2.1452 | −0.0353 | 2 | 0 |
| WCM2_Z3_1 | 2 | 41615 | 116573264 | 0.1135 | 0.5652 | 1.9452 | 1.0139 | 2 | 1 |
| WCM2_Z3_1 | 2 | 116593809 | 127413870 | −0.2028 | −0.1089 | 1.7631 | 0.0915 | 2 | 0 |
| WCM2_Z3_1 | 2 | 127414170 | 242946616 | 0.0996 | 0.5387 | 1.9666 | 0.9387 | 2 | 1 |
| WCM2_Z3_1 | 20 | 68373 | 60702744 | 0.148 | 0.63 | 1.9383 | 1.1568 | 2 | 1 |
| WCM2_Z3_1 | 20 | 60704003 | 62738025 | 0.2915 | 0.8859 | 1.8956 | 1.8003 | 2 | 2 |
| WCM2_Z3_1 | 20 | 62830252 | 62919090 | 0.0867 | 0.514 | NA | NA | NA | NA |
| WCM2_Z3_1 | 21 | 9907304 | 48084277 | −0.2033 | −0.11 | 0.9761 | 0.877 | 1 | 1 |
| WCM2_Z3_1 | 22 | 17063476 | 51215142 | −0.155 | 0.0046 | 1.0218 | 0.9846 | 1 | 1 |
| WCM2_Z3_1 | 3 | 239398 | 197894712 | 0.0886 | 0.5176 | 1.9334 | 0.9298 | 2 | 1 |
| WCM2_Z3_1 | 4 | 59345 | 36069616 | 0.1046 | 0.5482 | 1.8832 | 1.0413 | 2 | 1 |
| WCM2_Z3_1 | 4 | 36069833 | 52729651 | −0.5238 | −1.0653 | 0.9468 | 0.009 | 1 | 0 |
| WCM2_Z3_1 | 4 | 52740547 | 111543521 | 0.0702 | 0.482 | 1.8323 | 0.9611 | 2 | 1 |
| WCM2_Z3_1 | 4 | 111553577 | 190426502 | 0.2725 | 0.8532 | 2.7304 | 0.8825 | 3 | 1 |
| WCM2_Z3_1 | 5 | 140532 | 180687766 | 0.0651 | 0.4721 | 1.832 | 0.9422 | 2 | 1 |
| WCM2_Z3_1 | 6 | 203695 | 47645620 | 0.0786 | 0.4983 | 1.9492 | 0.8759 | 2 | 1 |
| WCM2_Z3_1 | 6 | 47646842 | 62284306 | −0.2125 | −0.1325 | 1.1696 | 0.6549 | 1 | 1 |
| WCM2_Z3_1 | 6 | 62390919 | 170893450 | 0.0257 | 0.394 | 1.8314 | 0.7967 | 2 | 1 |
| WCM2_Z3_1 | 7 | 193436 | 299850 | 0.308 | 0.9142 | NA | NA | NA | NA |
| WCM2_Z3_1 | 7 | 330227 | 71142262 | 0.3366 | 0.9626 | 1.975 | 1.9226 | 2 | 2 |
| WCM2_Z3_1 | 7 | 71175907 | 158937400 | 0.1004 | 0.5403 | 1.8852 | 1.0234 | 2 | 1 |
| WCM2_Z3_1 | 8 | 190908 | 29814844 | −0.1212 | 0.0819 | 2.1598 | −0.043 | 2 | 0 |
| WCM2_Z3_1 | 8 | 29921417 | 48885436 | 0.2048 | 0.7336 | 2.2545 | 1.0711 | 2 | 1 |
| WCM2_Z3_1 | 8 | 48887432 | 55612062 | 0.6175 | 1.4073 | NA | NA | NA | NA |
| WCM2_Z3_1 | 8 | 55614082 | 59483449 | 1.0097 | 1.9617 | NA | NA | NA | NA |
| WCM2_Z3_1 | 8 | 59484869 | 77775640 | 0.4688 | 1.1782 | 3.5943 | 0.9316 | 4 | 1 |
| WCM2_Z3_1 | 8 | 77775884 | 93329029 | 0.077 | 0.4951 | 1.538 | 1.2809 | 2 | 1 |
| WCM2_Z3_1 | 8 | 93897050 | 101727729 | 0.6764 | 1.4948 | 3.4895 | 2.1469 | 3 | 2 |
| WCM2_Z3_1 | 8 | 101730409 | 103541605 | 1.1004 | 2.082 | NA | NA | NA | NA |
| WCM2_Z3_1 | 8 | 103564040 | 104709502 | −0.1715 | −0.0339 | NA | NA | NA | NA |
| WCM2_Z3_1 | 8 | 104778567 | 110535519 | 0.4028 | 1.0721 | 2.1471 | 2.0579 | 2 | 2 |
| WCM2_Z3_1 | 8 | 110539186 | 117859924 | −0.1439 | 0.0302 | NA | NA | NA | NA |
| WCM2_Z3_1 | 8 | 117861249 | 124798817 | 0.751 | 1.6032 | NA | NA | NA | NA |
| WCM2_Z3_1 | 8 | 124799536 | 127568900 | 0.1948 | 0.7156 | NA | NA | NA | NA |
| WCM2_Z3_1 | 8 | 127569110 | 136657316 | −0.4889 | −0.9377 | 0.9304 | 0.1137 | 1 | 0 |
| WCM2_Z3_1 | 8 | 139144849 | 141745372 | 0.4083 | 1.081 | NA | NA | NA | NA |
| WCM2_Z3_1 | 8 | 141749130 | 146279545 | −0.472 | −0.8789 | 1.0317 | 0.0559 | 1 | 0 |
| WCM2_Z3_1 | 9 | 17116 | 7046917 | −0.5677 | −1.2383 | 0.884 | −0.0362 | 1 | 0 |
| WCM2_Z3_1 | 9 | 7049148 | 88968021 | −0.1546 | 0.0056 | 1.0435 | 0.9643 | 1 | 1 |
| WCM2_Z3_1 | 9 | 89560852 | 141111414 | 0.1543 | 0.6416 | 2.1631 | 0.9569 | 2 | 1 |
| WCM2_Z3_1 | X | 2707724 | 2836060 | 0.9315 | 1.856 | NA | NA | NA | NA |
| WCM2_Z3_1 | X | 2836211 | 27999311 | 0.9396 | 1.867 | NA | NA | NA | NA |
| WCM2_Z3_1 | X | 28807515 | 62885779 | 0.6368 | 1.4362 | NA | NA | NA | NA |
| WCM2_Z3_1 | X | 62898360 | 65428076 | 2.3608 | 3.5842 | NA | NA | NA | NA |
| WCM2_Z3_1 | X | 65474917 | 68381424 | 4.1876 | 5.5211 | NA | NA | NA | NA |
| WCM2_Z3_1 | X | 68381748 | 154509348 | −0.2302 | −0.1763 | NA | NA | NA | NA |
| WCM341 | 1 | 14930 | 156811634 | −0.1899 | 0.5685 | 1.9941 | 0.972 | 2 | 1 |
| WCM341 | 1 | 156812249 | 190067028 | 0.0301 | 1.0913 | 3.1105 | 1.1508 | 3 | 1 |
| WCM341 | 1 | 190067420 | 228473805 | −0.129 | 0.7246 | 2.1937 | 1.1111 | 2 | 1 |
| WCM341 | 1 | 228474437 | 248685768 | −0.0285 | 0.9621 | 2.8098 | 1.0865 | 3 | 1 |
| WCM341 | 1 | 248737474 | 249214028 | −0.24 | 0.4317 | NA | NA | NA | NA |
| WCM341 | 10 | 226107 | 5948533 | −0.0418 | 0.932 | 2.0429 | 1.7729 | 2 | 2 |
| WCM341 | 10 | 5951025 | 48025597 | 0.1238 | 1.2856 | 2.4379 | 2.4379 | 2 | 2 |
| WCM341 | 10 | 48033087 | 56106120 | 0.5175 | 1.9895 | 3.9709 | 3.9709 | 4 | 4 |
| WCM341 | 10 | 56129038 | 65928970 | 0.2837 | 1.59 | 3.0487 | 2.9723 | 3 | 3 |
| WCM341 | 10 | 65929489 | 79397240 | 0.5372 | 2.0212 | 4.0593 | 4.0593 | 4 | 4 |
| WCM341 | 10 | 79492257 | 79589199 | 0.198 | 1.4307 | NA | NA | NA | NA |
| WCM341 | 10 | 79590105 | 101667847 | −0.0316 | 0.9551 | 3.1584 | 0.7192 | 3 | 1 |
| WCM341 | 10 | 101668679 | 135235510 | −0.2309 | 0.4571 | 1.9001 | 0.8455 | 2 | 1 |
| WCM341 | 10 | 135235561 | 135477892 | −0.0684 | 0.8706 | NA | NA | NA | NA |
| WCM341 | 11 | 193112 | 65233857 | 0.1803 | 1.3967 | 2.633 | 2.633 | 3 | 3 |
| WCM341 | 11 | 65265435 | 65273799 | −0.9926 | NA | NA | NA | NA | NA |
| WCM341 | 11 | 65292653 | 69063668 | 0.12 | 1.278 | 2.425 | 2.425 | 2 | 2 |
| WCM341 | 11 | 69456225 | 71693951 | 1.5206 | 3.381 | 17.2626 | 3.573 | 17 | 4 |
| WCM341 | 11 | 71698125 | 134605959 | 0.1887 | 1.4128 | 2.6625 | 2.6625 | 3 | 3 |
| WCM341 | 12 | 148388 | 133810703 | −0.0415 | 0.9327 | 1.9088 | 1.9088 | 2 | 2 |
| WCM341 | 13 | 19041965 | 115089411 | 0.0285 | 1.0878 | 2.7293 | 1.5218 | 3 | 2 |
| WCM341 | 14 | 19110348 | 107283274 | 0.0921 | 1.2213 | 2.3316 | 2.3316 | 2 | 2 |
| WCM341 | 15 | 20083905 | 102506321 | −0.0357 | 0.9459 | 1.9264 | 1.9264 | 2 | 2 |
| WCM341 | 16 | 64424 | 68264699 | 0.1762 | 1.3887 | 2.6185 | 2.6185 | 3 | 3 |
| WCM341 | 16 | 68265322 | 90168857 | −0.3534 | 0.0864 | 2.2446 | −0.1211 | 2 | 0 |
| WCM341 | 17 | 263123 | 16046845 | −0.3825 | −0.0119 | 2.0272 | −0.0437 | 2 | 0 |
| WCM341 | 17 | 16049839 | 71205554 | −0.2091 | 0.5169 | 2.1447 | 0.7172 | 2 | 1 |
| WCM341 | 17 | 71205854 | 80541988 | −0.092 | 0.8148 | 1.8306 | 1.6876 | 2 | 2 |
| WCM341 | 17 | 80543738 | 80684986 | −0.215 | 0.5008 | NA | NA | NA | NA |
| WCM341 | 17 | 80696325 | 81187811 | −0.2834 | 0.3058 | 2.2567 | 0.2156 | 2 | 0 |
| WCM341 | 18 | 47714 | 78005194 | 0.0481 | 1.1296 | 2.7999 | 1.576 | 3 | 2 |
| WCM341 | 19 | 105021 | 42585292 | 0.0109 | 1.0496 | 2.6573 | 1.4825 | 3 | 1 |
| WCM341 | 19 | 42585512 | 45010215 | −0.1687 | 0.6239 | 1.7086 | 1.3735 | 2 | 1 |
| WCM341 | 19 | 45015182 | 48249031 | −0.0257 | 0.9686 | 2.5912 | 1.3225 | 3 | 1 |
| WCM341 | 19 | 48250324 | 58928786 | 0.5261 | 2.0033 | 5.1716 | 2.8469 | 5 | 3 |
| WCM341 | 19 | 58929052 | 59093696 | −0.1693 | 0.6224 | NA | NA | NA | NA |
| WCM341 | 2 | 41662 | 243037019 | −0.2044 | 0.5296 | 2.1441 | 0.743 | 2 | 1 |
| WCM341 | 20 | 68396 | 62934618 | 0.1518 | 1.3412 | 3.4502 | 1.6169 | 3 | 2 |
| WCM341 | 21 | 9590301 | 48084207 | −0.1174 | 0.7533 | 2.5785 | 0.7927 | 3 | 1 |
| WCM341 | 22 | 16159079 | 51219138 | 0.0342 | 1.1 | 2.4282 | 1.8588 | 2 | 2 |
| WCM341 | 3 | 239390 | 113655207 | 0.1324 | 1.3027 | 3.1845 | 1.7492 | 3 | 2 |
| WCM341 | 3 | 113656972 | 132075549 | −0.5012 | −0.474 | 1.4582 | −0.0182 | 1 | 0 |
| WCM341 | 3 | 132075945 | 197896800 | 0.1357 | 1.3094 | 3.1977 | 1.7591 | 3 | 2 |
| WCM341 | 4 | 53334 | 39458051 | −0.3611 | 0.0607 | 1.1024 | 0.9836 | 1 | 1 |
| WCM341 | 4 | 39459154 | 39460535 | −1.6505 | NA | NA | NA | NA | NA |
| WCM341 | 4 | 39460797 | 122137704 | −0.3403 | 0.1292 | 1.1291 | 1.0582 | 1 | 1 |
| WCM341 | 4 | 122148526 | 128865030 | −0.1355 | 0.7083 | 2.3687 | 0.8991 | 2 | 1 |
| WCM341 | 4 | 128865139 | 190903980 | −0.3611 | 0.0609 | 1.092 | 0.9942 | 1 | 1 |
| WCM341 | 5 | 90714 | 137759916 | −0.2332 | 0.4506 | 1.9274 | 0.8058 | 2 | 1 |
| WCM341 | 5 | 137760083 | 140857380 | −0.4037 | −0.087 | 1.5822 | 0.3008 | 2 | 0 |
| WCM341 | 5 | 140857868 | 180687812 | −0.2291 | 0.4622 | 1.9362 | 0.819 | 2 | 1 |
| WCM341 | 6 | 203428 | 66115281 | 0.113 | 1.2638 | 2.862 | 1.9406 | 3 | 2 |
| WCM341 | 6 | 66200400 | 170892877 | −0.0537 | 0.9047 | 2.9003 | 0.8438 | 3 | 1 |
| WCM341 | 7 | 193222 | 158937142 | 0.3403 | 1.6911 | 3.2291 | 3.2291 | 3 | 3 |
| WCM341 | 8 | 163527 | 29786035 | 0.1373 | 1.3126 | 3.9635 | 1.0042 | 4 | 1 |
| WCM341 | 8 | 29786342 | 146279529 | 0.0563 | 1.147 | 4.406 | 0.023 | 4 | 0 |
| WCM341 | 9 | 14903 | 2060789 | 0.246 | 1.521 | 2.923 | 2.8167 | 3 | 3 |
| WCM341 | 9 | 2070400 | 36372494 | 0.332 | 1.6764 | 3.1963 | 3.1963 | 3 | 3 |
| WCM341 | 9 | 36375867 | 69847116 | −0.0088 | 1.0064 | 3.1543 | 0.8634 | 3 | 1 |
| WCM341 | 9 | 69848036 | 141107539 | 0.294 | 1.6086 | 4.358 | 1.7411 | 4 | 2 |
| WCM341 | X | 2700090 | 63574833 | 0.1086 | 1.255 | NA | NA | NA | NA |
| WCM341 | X | 63576002 | 68062026 | 1.754 | 3.6641 | NA | NA | NA | NA |
| WCM341 | X | 68381140 | 154493589 | 0.0764 | 1.1889 | NA | NA | NA | NA |
| WCM341_X1_1_p2 | 1 | 69594 | 43149117 | −0.1499 | −0.9346 | 0.7984 | 0.2479 | 1 | 0 |
| WCM341_X1_1_p2 | 1 | 43161872 | 118617164 | −0.2548 | −1.2742 | 0.6461 | 0.1808 | 1 | 0 |
| WCM341_X1_1_p2 | 1 | 118624123 | 145561594 | −0.1967 | −1.0792 | 0.9169 | 0.0296 | 1 | 0 |
| WCM341_X1_1_p2 | 1 | 145561750 | 158047869 | −0.2375 | −1.2142 | 0.6739 | 0.1882 | 1 | 0 |
| WCM341_X1_1_p2 | 1 | 158054237 | 249212369 | −0.08 | −0.735 | 0.9045 | 0.2971 | 1 | 0 |
| WCM341_X1_1_p2 | 10 | 255922 | 3181071 | −0.0896 | −0.7613 | NA | NA | NA | NA |
| WCM341_X1_1_p2 | 10 | 3182988 | 48033075 | 0.0771 | −0.3407 | 0.991 | 0.5883 | 1 | 1 |
| WCM341_X1_1_p2 | 10 | 48370643 | 79397279 | 0.3428 | 0.2139 | 1.1796 | 1.14 | 1 | 1 |
| WCM341_X1_1_p2 | 10 | 79552234 | 135440117 | −0.0959 | −0.7789 | 0.8702 | 0.2954 | 1 | 0 |
| WCM341_X1_1_p2 | 11 | 193112 | 134856534 | 0.1357 | −0.2083 | 0.8846 | 0.8465 | 1 | 1 |
| WCM341_X1_1_p2 | 12 | 148639 | 133810703 | −0.0222 | −0.5822 | 0.8115 | 0.5244 | 1 | 1 |
| WCM341_X1_1_p2 | 13 | 19600515 | 115089411 | 0.001 | −0.5235 | 0.9301 | 0.4613 | 1 | 0 |
| WCM341_X1_1_p2 | 14 | 19377689 | 107283055 | −0.045 | −0.6412 | 0.6715 | 0.6109 | 1 | 1 |
| WCM341_X1_1_p2 | 15 | 20170106 | 102506273 | −0.1464 | −0.9242 | 0.6602 | 0.3937 | 1 | 0 |
| WCM341_X1_1_p2 | 16 | 67604 | 68224794 | 0.0257 | −0.4626 | 0.7522 | 0.6992 | 1 | 1 |
| WCM341_X1_1_p2 | 16 | 68225005 | 90233312 | −0.3508 | −1.6446 | 0.6518 | −0.0122 | 1 | 0 |
| WCM341_X1_1_p2 | 17 | 131595 | 16330085 | −0.3376 | −1.5891 | 0.7045 | −0.0398 | 1 | 0 |
| WCM341_X1_1_p2 | 17 | 16330769 | 80847561 | −0.2189 | −1.1514 | 0.6365 | 0.2638 | 1 | 0 |
| WCM341_X1_1_p2 | 17 | 80851476 | 81083561 | −0.3874 | −1.807 | 0.5405 | 0.0311 | 1 | 0 |
| WCM341_X1_1_p2 | 18 | 48220 | 78005215 | −0.1033 | −0.7995 | 0.7183 | 0.4308 | 1 | 0 |
| WCM341_X1_1_p2 | 19 | 281467 | 42621153 | −0.0866 | −0.7529 | 0.7797 | 0.4071 | 1 | 0 |
| WCM341_X1_1_p2 | 19 | 42621545 | 43699410 | −0.36 | −1.6841 | NA | NA | NA | NA |
| WCM341_X1_1_p2 | 19 | 43708085 | 48202609 | −0.0924 | −0.7691 | 0.7512 | 0.4224 | 1 | 0 |
| WCM341_X1_1_p2 | 19 | 48204609 | 58944877 | 0.2655 | 0.0635 | 1.2703 | 0.8197 | 1 | 1 |
| WCM341_X1_1_p2 | 19 | 58945079 | 59083929 | −0.0133 | −0.5595 | NA | NA | NA | NA |
| WCM341_X1_1_p2 | 2 | 41612 | 242946607 | −0.2509 | −1.2604 | 0.6405 | 0.1943 | 1 | 0 |
| WCM341_X1_1_p2 | 20 | 68396 | 62904863 | 0.074 | −0.3477 | 1.111 | 0.4607 | 1 | 0 |
| WCM341_X1_1_p2 | 21 | 9826205 | 48084204 | −0.1855 | −1.0437 | 0.688 | 0.2821 | 1 | 0 |
| WCM341_X1_1_p2 | 22 | 17063496 | 51215137 | −0.0462 | −0.6445 | 0.8531 | 0.4263 | 1 | 0 |
| WCM341_X1_1_p2 | 3 | 239349 | 113684048 | 0.0042 | −0.5157 | 0.9744 | 0.4245 | 1 | 0 |
| WCM341_X1_1_p2 | 3 | 113697110 | 132051081 | −0.4952 | −2.3938 | 0.4316 | −0.051 | 0 | 0 |
| WCM341_X1_1_p2 | 3 | 132056357 | 197894713 | 0.0042 | −0.5156 | 0.9257 | 0.4733 | 1 | 0 |
| WCM341_X1_1_p2 | 4 | 59326 | 190426459 | −0.2529 | −1.2673 | 0.5742 | 0.2567 | 1 | 0 |
| WCM341_X1_1_p2 | 5 | 140532 | 180687812 | −0.1327 | −0.8838 | 0.7572 | 0.3267 | 1 | 0 |
| WCM341_X1_1_p2 | 6 | 203510 | 68598936 | 0.0803 | −0.3333 | 1.0121 | 0.5754 | 1 | 1 |
| WCM341_X1_1_p2 | 6 | 69348790 | 170852725 | −0.0167 | −0.5681 | 1.0827 | 0.2663 | 1 | 0 |
| WCM341_X1_1_p2 | 6 | 170855270 | 170892767 | 0.457 | 0.4235 | NA | NA | NA | NA |
| WCM341_X1_1_p2 | 7 | 193222 | 158937013 | 0.3315 | 0.1924 | 1.1667 | 1.1186 | 1 | 1 |
| WCM341_X1_1_p2 | 8 | 190900 | 30040659 | 0.2092 | −0.0511 | 1.5429 | 0.3875 | 2 | 0 |
| WCM341_X1_1_p2 | 8 | 30209634 | 146157248 | 0.1152 | −0.2538 | 1.5829 | 0.0944 | 2 | 0 |
| WCM341_X1_1_p2 | 8 | 146157542 | 146279513 | 0.1947 | −0.0813 | NA | NA | NA | NA |
| WCM341_X1_1_p2 | 9 | 17055 | 36219880 | 0.3161 | 0.1629 | 1.1865 | 1.0526 | 1 | 1 |
| WCM341_X1_1_p2 | 9 | 36222887 | 140901323 | 0.2463 | 0.0249 | 1.5094 | 0.5255 | 2 | 1 |
| WCM341_X1_1_p2 | 9 | 140904475 | 141111414 | 0.2214 | −0.0258 | NA | NA | NA | NA |
| WCM341_X1_1_p2 | X | 2700151 | 65486388 | 0.1255 | −0.2308 | NA | NA | NA | NA |
| WCM341_X1_1_p2 | X | 65819482 | 68725979 | 3.2749 | 3.9778 | NA | NA | NA | NA |
| WCM341_X1_1_p2 | X | 68748871 | 154509348 | 0.0991 | −0.2901 | NA | NA | NA | NA |
| WCM341_Z2_1 | 1 | 69635 | 157103911 | −0.2593 | −0.5891 | 0.9757 | 0.3538 | 1 | 0 |
| WCM341_Z2_1 | 1 | 157105454 | 183913347 | −0.0336 | −0.2569 | 1.292 | 0.3818 | 1 | 0 |
| WCM341_Z2_1 | 1 | 183914597 | 229738171 | −0.2223 | −0.5329 | 1.0133 | 0.369 | 1 | 0 |
| WCM341_Z2_1 | 1 | 229738236 | 248366749 | −0.0152 | −0.2309 | 1.3443 | 0.3599 | 1 | 0 |
| WCM341_Z2_1 | 1 | 248366980 | 249212373 | 0.0167 | −0.1861 | NA | NA | NA | NA |
| WCM341_Z2_1 | 10 | 255851 | 4884039 | −0.1838 | −0.4752 | NA | NA | NA | NA |
| WCM341_Z2_1 | 10 | 4884636 | 47701275 | 0.0988 | −0.0724 | 1.0956 | 0.8065 | 1 | 1 |
| WCM341_Z2_1 | 10 | 47769327 | 50667132 | 0.4265 | 0.3597 | 1.3126 | 1.2538 | 1 | 1 |
| WCM341_Z2_1 | 10 | 50668448 | 50678717 | −1.0272 | −2.0268 | NA | NA | NA | NA |
| WCM341_Z2_1 | 10 | 50678858 | 78868313 | 0.52 | 0.4778 | 1.4118 | 1.3734 | 1 | 1 |
| WCM341_Z2_1 | 10 | 78870062 | 79397279 | 0.0231 | −0.177 | NA | NA | NA | NA |
| WCM341_Z2_1 | 10 | 79552256 | 106027158 | −0.1221 | −0.3843 | 1.2042 | 0.3281 | 1 | 0 |
| WCM341_Z2_1 | 10 | 106028094 | 135440105 | −0.4005 | −0.8108 | 0.9958 | 0.1444 | 1 | 0 |
| WCM341_Z2_1 | 11 | 193112 | 14246296 | 0.2467 | 0.1265 | 1.2576 | 0.9257 | 1 | 1 |
| WCM341_Z2_1 | 11 | 14264916 | 19596868 | 0.6198 | 0.6017 | 2.009 | 1.026 | 2 | 1 |
| WCM341_Z2_1 | 11 | 19735325 | 134856536 | 0.2019 | 0.0671 | 1.0616 | 1.0336 | 1 | 1 |
| WCM341_Z2_1 | 12 | 148811 | 133810703 | −0.0524 | −0.2837 | 0.9681 | 0.6749 | 1 | 1 |
| WCM341_Z2_1 | 13 | 19600515 | 115089411 | 0.1136 | −0.0522 | 1.2776 | 0.6513 | 1 | 1 |
| WCM341_Z2_1 | 14 | 20201875 | 45711700 | 0.2692 | 0.1562 | 1.1378 | 1.0909 | 1 | 1 |
| WCM341_Z2_1 | 14 | 45712000 | 106361508 | 0.1291 | −0.0311 | 0.9933 | 0.9641 | 1 | 1 |
| WCM341_Z2_1 | 14 | 106363825 | 107283063 | 0.249 | 0.1296 | NA | NA | NA | NA |
| WCM341_Z2_1 | 15 | 20170117 | 102389747 | −0.0617 | −0.297 | 0.9829 | 0.645 | 1 | 1 |
| WCM341_Z2_1 | 16 | 67604 | 68217219 | 0.1987 | 0.0627 | 1.0626 | 1.0263 | 1 | 1 |
| WCM341_Z2_1 | 16 | 68224911 | 90233297 | −0.513 | −0.9973 | 0.9629 | 0.039 | 1 | 0 |
| WCM341_Z2_1 | 17 | 131595 | 15973601 | −0.5134 | −0.9981 | 0.965 | 0.0363 | 1 | 0 |
| WCM341_Z2_1 | 17 | 15973823 | 74309955 | −0.2676 | −0.6017 | 0.951 | 0.367 | 1 | 0 |
| WCM341_Z2_1 | 17 | 74324870 | 80786553 | −0.0781 | −0.3205 | 0.9529 | 0.6487 | 1 | 1 |
| WCM341_Z2_1 | 17 | 80788219 | 81083561 | −0.2913 | −0.6383 | 0.9615 | 0.3235 | 1 | 0 |
| WCM341_Z2_1 | 18 | 48212 | 78005224 | 0.0082 | −0.1979 | 1.0455 | 0.6981 | 1 | 1 |
| WCM341_Z2_1 | 19 | 281445 | 42603965 | 0.0437 | −0.1484 | 1.1329 | 0.6716 | 1 | 1 |
| WCM341_Z2_1 | 19 | 42621152 | 42914965 | −0.7954 | −1.517 | NA | NA | NA | NA |
| WCM341_Z2_1 | 19 | 42927572 | 48253479 | 0.0036 | −0.2044 | 1.052 | 0.6838 | 1 | 1 |
| WCM341_Z2_1 | 19 | 48254331 | 58929052 | 0.634 | 0.6192 | 1.8531 | 1.219 | 2 | 1 |
| WCM341_Z2_1 | 19 | 58943692 | 59083951 | −0.0439 | −0.2715 | NA | NA | NA | NA |
| WCM341_Z2_1 | 2 | 41620 | 242946597 | −0.2574 | −0.5861 | 0.9785 | 0.3538 | 1 | 0 |
| WCM341_Z2_1 | 20 | 68373 | 62904847 | 0.2753 | 0.1643 | 1.574 | 0.6672 | 2 | 1 |
| WCM341_Z2_1 | 21 | 9826225 | 48084281 | −0.1307 | −0.3968 | 1.1528 | 0.3663 | 1 | 0 |
| WCM341_Z2_1 | 22 | 17063496 | 51215130 | 0.0705 | −0.1113 | 1.2387 | 0.6129 | 1 | 1 |
| WCM341_Z2_1 | 3 | 239472 | 113664267 | 0.22 | 0.0912 | 1.4747 | 0.6559 | 1 | 1 |
| WCM341_Z2_1 | 3 | 113667680 | 131624150 | −0.7761 | −1.4786 | 0.6983 | 0.0194 | 1 | 0 |
| WCM341_Z2_1 | 3 | 131756412 | 197894719 | 0.2444 | 0.1235 | 1.4698 | 0.709 | 1 | 1 |
| WCM341_Z2_1 | 4 | 59331 | 122687238 | −0.4837 | −0.9478 | 0.6828 | 0.354 | 1 | 0 |
| WCM341_Z2_1 | 4 | 122687482 | 128996108 | −0.1997 | −0.499 | 1.055 | 0.3603 | 1 | 0 |
| WCM341_Z2_1 | 4 | 128999075 | 190426459 | −0.4728 | −0.9295 | 0.681 | 0.369 | 1 | 0 |
| WCM341_Z2_1 | 5 | 140532 | 180687812 | −0.2629 | −0.5945 | 0.9694 | 0.3551 | 1 | 0 |
| WCM341_Z2_1 | 6 | 203583 | 66014528 | 0.1134 | −0.0524 | 1.288 | 0.6407 | 1 | 1 |
| WCM341_Z2_1 | 6 | 66042231 | 170892615 | −0.0473 | −0.2764 | 1.2871 | 0.3643 | 1 | 0 |
| WCM341_Z2_1 | 7 | 193205 | 158937161 | 0.5524 | 0.5183 | 1.5642 | 1.3003 | 2 | 1 |
| WCM341_Z2_1 | 8 | 190908 | 30510964 | 0.3667 | 0.2831 | 2.0087 | 0.4249 | 2 | 0 |
| WCM341_Z2_1 | 8 | 30537041 | 92088822 | 0.1887 | 0.0494 | 1.9913 | 0.0784 | 2 | 0 |
| WCM341_Z2_1 | 8 | 92090727 | 97307346 | 0.7705 | 0.7852 | NA | NA | NA | NA |
| WCM341_Z2_1 | 8 | 97311955 | 146279481 | 0.1615 | 0.0128 | 1.9733 | 0.0445 | 2 | 0 |
| WCM341_Z2_1 | 9 | 17058 | 36170461 | 0.5139 | 0.4702 | 1.5885 | 1.1822 | 2 | 1 |
| WCM341_Z2_1 | 9 | 36170642 | 141111414 | 0.4121 | 0.3414 | 1.8965 | 0.6374 | 2 | 1 |
| WCM341_Z2_1 | X | 2700148 | 65486378 | 0.2507 | 0.1318 | NA | NA | NA | NA |
| WCM341_Z2_1 | X | 65819482 | 68725979 | 4.2131 | 4.4728 | NA | NA | NA | NA |
| WCM341_Z2_1 | X | 68748875 | 154509348 | 0.22 | 0.0912 | NA | NA | NA | NA |
| WCM365 | 1 | 14930 | 16817454 | −0.3978 | 0.0418 | 1.9956 | 0.0632 | 2 | 0 |
| WCM365 | 1 | 16817690 | 17272777 | −0.6134 | −0.3473 | 1.449 | 0.1231 | 1 | 0 |
| WCM365 | 1 | 17273325 | 143391598 | −0.4233 | −0.0019 | 1.9634 | 0.0339 | 2 | 0 |
| WCM365 | 1 | 143391861 | 156351113 | 0.3119 | 1.1001 | 2.6485 | 1.639 | 3 | 2 |
| WCM365 | 1 | 156351699 | 205538350 | 0.4357 | 1.2634 | 2.7069 | 2.0944 | 3 | 2 |
| WCM365 | 1 | 205548875 | 225602998 | 0.3421 | 1.1405 | 2.2162 | 2.1928 | 2 | 2 |
| WCM365 | 1 | 225605715 | 228873445 | −0.1625 | 0.4223 | 2.6859 | −0.0058 | 3 | 0 |
| WCM365 | 1 | 228879028 | 230933898 | 0.5168 | 1.3682 | 2.6112 | 2.5517 | 3 | 3 |
| WCM365 | 1 | 230972981 | 235491896 | −0.2 | 0.3641 | 2.6185 | −0.0443 | 3 | 0 |
| WCM365 | 1 | 235498675 | 249213464 | 0.523 | 1.3762 | 2.5957 | 2.5957 | 3 | 3 |
| WCM365 | 10 | 226118 | 35099886 | −0.5124 | −0.1591 | 1.7897 | 0.0015 | 2 | 0 |
| WCM365 | 10 | 35103904 | 35896974 | 0.5731 | 1.4399 | NA | NA | NA | NA |
| WCM365 | 10 | 35897346 | 65930362 | 0.2447 | 1.0095 | 2.0132 | 2.0132 | 2 | 2 |
| WCM365 | 10 | 66473307 | 84625107 | 0.735 | 1.6421 | 4.171 | 2.0714 | 4 | 2 |
| WCM365 | 10 | 84711187 | 89550252 | −0.4238 | −0.0028 | 2.0004 | −0.0043 | 2 | 0 |
| WCM365 | 10 | 89552520 | 89728379 | −1.8756 | NA | NA | NA | NA | NA |
| WCM365 | 10 | 90034829 | 135477898 | −0.413 | 0.0159 | 1.9828 | 0.0394 | 2 | 0 |
| WCM365 | 11 | 193096 | 2893001 | 0.1509 | 0.8804 | 2.2564 | 1.4253 | 2 | 1 |
| WCM365 | 11 | 2905000 | 36680581 | 0.0594 | 0.7513 | 1.6833 | 1.6833 | 2 | 2 |
| WCM365 | 11 | 39882942 | 62286373 | 0.1896 | 0.9342 | 2.414 | 1.4075 | 2 | 1 |
| WCM365 | 11 | 62286825 | 62652204 | −0.1488 | 0.4433 | NA | NA | NA | NA |
| WCM365 | 11 | 62652779 | 66613043 | 0.1591 | 0.8919 | 1.8556 | 1.8556 | 2 | 2 |
| WCM365 | 11 | 66613575 | 68131420 | 0.9582 | 1.9124 | 5.5061 | 2.0229 | 6 | 2 |
| WCM365 | 11 | 68133067 | 69997146 | 1.4475 | 2.4795 | 9.0365 | 2.1177 | 9 | 2 |
| WCM365 | 11 | 69998546 | 71276632 | 1.1366 | 2.1228 | 6.5039 | 2.2067 | 7 | 2 |
| WCM365 | 11 | 71276809 | 90602334 | −0.0672 | 0.5666 | 1.889 | 1.0731 | 2 | 1 |
| WCM365 | 11 | 92085375 | 107650507 | 0.1396 | 0.8647 | 1.821 | 1.821 | 2 | 2 |
| WCM365 | 11 | 107661831 | 109296234 | −0.2182 | 0.3357 | 1.7744 | 0.7495 | 2 | 1 |
| WCM365 | 11 | 110001861 | 111431614 | 0.3328 | 1.1281 | 2.2089 | 2.1624 | 2 | 2 |
| WCM365 | 11 | 111473353 | 125525358 | −0.4004 | 0.0375 | 1.9733 | 0.0793 | 2 | 0 |
| WCM365 | 11 | 125542510 | 134605853 | −0.0099 | 0.6512 | 1.6646 | 1.4764 | 2 | 1 |
| WCM365 | 12 | 148576 | 20903865 | −0.0874 | 0.5364 | 1.994 | 0.9067 | 2 | 1 |
| WCM365 | 12 | 20905250 | 43887093 | 0.2258 | 0.9837 | 1.983 | 1.9722 | 2 | 2 |
| WCM365 | 12 | 43895927 | 65641123 | −0.0421 | 0.6038 | 1.5198 | 1.5198 | 2 | 2 |
| WCM365 | 12 | 65641544 | 133733463 | 0.1823 | 0.924 | 1.9341 | 1.8607 | 2 | 2 |
| WCM365 | 12 | 133758509 | 133810794 | −0.0452 | 0.5993 | NA | NA | NA | NA |
| WCM365 | 13 | 19042044 | 20660818 | 0.1325 | 0.8547 | 1.8616 | 1.7552 | 2 | 2 |
| WCM365 | 13 | 20715539 | 115089411 | −0.3752 | 0.0802 | 2.0013 | 0.1131 | 2 | 0 |
| WCM365 | 14 | 19110297 | 38016880 | 0.1303 | 0.8517 | 2.4368 | 1.1724 | 2 | 1 |
| WCM365 | 14 | 38019959 | 38680104 | −0.241 | 0.2996 | NA | NA | NA | NA |
| WCM365 | 14 | 38680352 | 89154832 | −0.421 | 0.0021 | 1.0014 | 1.0014 | 1 | 1 |
| WCM365 | 14 | 89160728 | 97347512 | −0.1357 | 0.4634 | 1.5597 | 1.198 | 2 | 1 |
| WCM365 | 14 | 97410013 | 107283263 | 0.1927 | 0.9384 | 2.8141 | 1.0186 | 3 | 1 |
| WCM365 | 15 | 20083928 | 67008737 | −0.1314 | 0.4699 | 1.9124 | 0.8578 | 2 | 1 |
| WCM365 | 15 | 67010033 | 69689748 | −0.8674 | −0.8829 | 1.1628 | −0.0783 | 1 | 0 |
| WCM365 | 15 | 69692440 | 102506270 | −0.0661 | 0.5683 | 1.891 | 1.0745 | 2 | 1 |
| WCM365 | 16 | 64426 | 51680598 | 0.1383 | 0.8629 | 1.8187 | 1.8187 | 2 | 2 |
| WCM365 | 16 | 52289317 | 90260461 | −0.4357 | −0.0232 | 1.9406 | 0.0275 | 2 | 0 |
| WCM365 | 17 | 131595 | 455063 | 0.3647 | 1.1704 | NA | NA | NA | NA |
| WCM365 | 17 | 456727 | 6900189 | 0.1641 | 0.8988 | 3.6507 | 0.0784 | 4 | 0 |
| WCM365 | 17 | 6900362 | 10267863 | −0.5209 | −0.1746 | 1.7781 | −0.006 | 2 | 0 |
| WCM365 | 17 | 10293908 | 25309007 | 0.1436 | 0.8702 | 1.8433 | 1.8126 | 2 | 2 |
| WCM365 | 17 | 25621347 | 30821990 | −0.078 | 0.5506 | 1.6967 | 1.2327 | 2 | 1 |
| WCM365 | 17 | 30921534 | 36093849 | 0.137 | 0.861 | 1.9383 | 1.6944 | 2 | 2 |
| WCM365 | 17 | 36099546 | 41180046 | −0.0279 | 0.6247 | 1.5419 | 1.5419 | 2 | 2 |
| WCM365 | 17 | 41180451 | 41296245 | 0.1477 | 0.876 | NA | NA | NA | NA |
| WCM365 | 17 | 41323203 | 42478819 | −0.5168 | −0.1672 | 1.8335 | −0.0524 | 2 | 0 |
| WCM365 | 17 | 42483233 | 45886695 | −0.1431 | 0.452 | 1.5667 | 1.1692 | 2 | 1 |
| WCM365 | 17 | 45888097 | 48918385 | 0.3738 | 1.1824 | 2.4791 | 2.06 | 2 | 2 |
| WCM365 | 17 | 48939746 | 64001883 | −0.1269 | 0.4768 | 1.6519 | 1.1314 | 2 | 1 |
| WCM365 | 17 | 64023624 | 73760724 | 0.1629 | 0.8972 | 1.8625 | 1.8625 | 2 | 2 |
| WCM365 | 17 | 73761057 | 73805973 | −0.9231 | −1.0165 | NA | NA | NA | NA |
| WCM365 | 17 | 73806075 | 81187930 | 0.1535 | 0.884 | 1.8455 | 1.8455 | 2 | 2 |
| WCM365 | 18 | 47793 | 199244 | 0.2547 | 1.0231 | NA | NA | NA | NA |
| WCM365 | 18 | 203081 | 2674182 | 0.5629 | 1.4269 | 3.1222 | 2.2553 | 3 | 2 |
| WCM365 | 18 | 2688513 | 3277944 | 1.0521 | 2.0238 | NA | NA | NA | NA |
| WCM365 | 18 | 3447814 | 14147414 | 0.5877 | 1.4583 | 3.4319 | 2.0639 | 3 | 2 |
| WCM365 | 18 | 14149969 | 24081139 | 0.1701 | 0.9071 | 1.9065 | 1.8441 | 2 | 2 |
| WCM365 | 18 | 24126753 | 67997305 | −0.4097 | 0.0216 | 1.9995 | 0.0307 | 2 | 0 |
| WCM365 | 18 | 70204111 | 78005240 | 0.1266 | 0.8465 | 1.8359 | 1.7602 | 2 | 2 |
| WCM365 | 19 | 105021 | 3595522 | −0.501 | −0.1387 | 1.8013 | 0.0154 | 2 | 0 |
| WCM365 | 19 | 3595794 | 4053998 | 0.3845 | 1.1965 | 2.4004 | 2.1833 | 2 | 2 |
| WCM365 | 19 | 4054772 | 5592938 | 0.7179 | 1.621 | 4.1703 | 1.9815 | 4 | 2 |
| WCM365 | 19 | 5595369 | 10073478 | 0.3982 | 1.2145 | 2.5519 | 2.0891 | 3 | 2 |
| WCM365 | 19 | 10077158 | 15752323 | −0.112 | 0.4994 | 1.7685 | 1.0587 | 2 | 1 |
| WCM365 | 19 | 15752423 | 18499149 | 0.2144 | 0.9682 | 1.9564 | 1.9564 | 2 | 2 |
| WCM365 | 19 | 18502834 | 23542752 | 0.4159 | 1.2377 | 2.6079 | 2.1086 | 3 | 2 |
| WCM365 | 19 | 23543100 | 39109975 | 0.1467 | 0.8746 | 1.8335 | 1.8335 | 2 | 2 |
| WCM365 | 19 | 39111060 | 43262094 | −0.507 | −0.1495 | 1.8055 | −0.0023 | 2 | 0 |
| WCM365 | 19 | 43262444 | 43544495 | −1.6236 | −5.2873 | NA | NA | NA | NA |
| WCM365 | 19 | 43568739 | 44896664 | −0.4029 | 0.0332 | 2.014 | 0.0325 | 2 | 0 |
| WCM365 | 19 | 44901402 | 47030313 | 0.2977 | 1.0812 | 4.2769 | −0.0454 | 4 | 0 |
| WCM365 | 19 | 47032047 | 50399231 | 0.0126 | 0.6839 | 3.2519 | −0.0389 | 3 | 0 |
| WCM365 | 19 | 50399335 | 51430398 | 0.2421 | 1.006 | 3.8391 | 0.1776 | 4 | 0 |
| WCM365 | 19 | 51430652 | 54265616 | 0.6193 | 1.4981 | 3.712 | 1.9375 | 4 | 2 |
| WCM365 | 19 | 54291049 | 58437644 | 1.135 | 2.121 | 6.5418 | 2.1579 | 7 | 2 |
| WCM365 | 19 | 58437851 | 58992002 | 1.5873 | 2.6365 | 10.5227 | 1.914 | 11 | 2 |
| WCM365 | 19 | 59009984 | 59093696 | −0.2677 | 0.2569 | NA | NA | NA | NA |
| WCM365 | 2 | 41583 | 84804515 | −0.0343 | 0.6154 | 1.8004 | 1.2635 | 2 | 1 |
| WCM365 | 2 | 84804548 | 88482514 | 0.3079 | 1.0947 | 2.2902 | 1.9813 | 2 | 2 |
| WCM365 | 2 | 88484945 | 97613616 | −0.07 | 0.5625 | 1.5913 | 1.3624 | 2 | 1 |
| WCM365 | 2 | 97617140 | 196619240 | −0.158 | 0.4293 | 1.5516 | 1.1416 | 2 | 1 |
| WCM365 | 2 | 196620991 | 207395744 | −0.0317 | 0.6191 | 1.9518 | 1.1201 | 2 | 1 |
| WCM365 | 2 | 207406890 | 210010180 | 0.3133 | 1.1021 | 2.8902 | 1.4031 | 3 | 1 |
| WCM365 | 2 | 210343638 | 243037004 | −0.0273 | 0.6257 | 1.9714 | 1.1144 | 2 | 1 |
| WCM365 | 20 | 68397 | 25667039 | −0.4221 | 3.00E−04 | 1.9451 | 0.0553 | 2 | 0 |
| WCM365 | 20 | 25733312 | 47888117 | 0.235 | 0.9963 | 1.9949 | 1.9949 | 2 | 2 |
| WCM365 | 20 | 47892331 | 47905508 | −0.9816 | −1.1653 | NA | NA | NA | NA |
| WCM365 | 20 | 47988701 | 62934541 | 0.2712 | 1.0455 | 2.0641 | 2.0641 | 2 | 2 |
| WCM365 | 21 | 9590305 | 39903442 | −0.3022 | 0.2012 | 1.1946 | 1.1047 | 1 | 1 |
| WCM365 | 21 | 39906065 | 42870017 | −0.7751 | −0.6759 | 1.2281 | 0.0238 | 1 | 0 |
| WCM365 | 21 | 43133094 | 48084177 | −0.2262 | 0.323 | 1.2589 | 1.2431 | 1 | 1 |
| WCM365 | 22 | 16159060 | 41734331 | −0.4255 | −0.0057 | 1.9766 | 0.0155 | 2 | 0 |
| WCM365 | 22 | 41734486 | 42896725 | −0.0976 | 0.5212 | 2.8947 | −0.0245 | 3 | 0 |
| WCM365 | 22 | 42898736 | 51219089 | −0.4112 | 0.0189 | 1.9739 | 0.0524 | 2 | 0 |
| WCM365 | 3 | 239524 | 85561088 | −0.0884 | 0.535 | 1.8509 | 1.0469 | 2 | 1 |
| WCM365 | 3 | 85757553 | 118866376 | 0.0799 | 0.7806 | 1.7398 | 1.6959 | 2 | 2 |
| WCM365 | 3 | 118867047 | 197785515 | 0.1021 | 0.8119 | 2.3755 | 1.1356 | 2 | 1 |
| WCM365 | 3 | 197785811 | 197896678 | 0.131 | 0.8526 | NA | NA | NA | NA |
| WCM365 | 4 | 53341 | 2438561 | −0.0918 | 0.5298 | 2.9093 | −0.0217 | 3 | 0 |
| WCM365 | 4 | 2439900 | 2691258 | 0.013 | 0.6844 | NA | NA | NA | NA |
| WCM365 | 4 | 2692454 | 21950259 | 0.1857 | 0.9288 | 2.7367 | 1.0706 | 3 | 1 |
| WCM365 | 4 | 22389499 | 49063783 | 0.0348 | 0.7159 | 2.2342 | 1.0509 | 2 | 1 |
| WCM365 | 4 | 49064001 | 49561719 | −1.2987 | −2.2175 | NA | NA | NA | NA |
| WCM365 | 4 | 49601562 | 54969875 | 0.0769 | 0.7763 | NA | NA | NA | NA |
| WCM365 | 4 | 55125035 | 55606728 | 0.6479 | 1.534 | NA | NA | NA | NA |
| WCM365 | 4 | 55946171 | 190904468 | 0.0193 | 0.6936 | 2.2027 | 1.0319 | 2 | 1 |
| WCM365 | 5 | 90784 | 181826 | 0.3374 | 1.1342 | NA | NA | NA | NA |
| WCM365 | 5 | 182291 | 3599698 | 0.9204 | 1.8674 | 5.1704 | 2.1269 | 5 | 2 |
| WCM365 | 5 | 3599857 | 52404485 | 0.3893 | 1.2028 | 2.3019 | 2.3019 | 2 | 2 |
| WCM365 | 5 | 52404546 | 64538001 | −0.3708 | 0.0876 | 1.0662 | 1.059 | 1 | 1 |
| WCM365 | 5 | 64556485 | 67593446 | −0.0793 | 0.5486 | 1.8781 | 1.0471 | 2 | 1 |
| WCM365 | 5 | 67593968 | 92919714 | −0.4203 | 0.0034 | 1.0024 | 1.0024 | 1 | 1 |
| WCM365 | 5 | 92921032 | 96514921 | −0.097 | 0.5221 | 1.5246 | 1.3475 | 2 | 1 |
| WCM365 | 5 | 96518765 | 131411722 | −0.4121 | 0.0174 | 1.0122 | 1.0122 | 1 | 1 |
| WCM365 | 5 | 131528316 | 133308560 | −0.9314 | −1.0371 | 1.0094 | −0.0348 | 1 | 0 |
| WCM365 | 5 | 133309581 | 145872599 | −0.3443 | 0.1319 | 1.1066 | 1.0848 | 1 | 1 |
| WCM365 | 5 | 145878058 | 180687776 | 0.0485 | 0.7357 | 1.6699 | 1.6604 | 2 | 2 |
| WCM365 | 6 | 203546 | 22147301 | −0.0165 | 0.6414 | 1.5987 | 1.521 | 2 | 2 |
| WCM365 | 6 | 22287685 | 26017582 | 0.2958 | 1.0786 | 2.112 | 2.112 | 2 | 2 |
| WCM365 | 6 | 26017895 | 32139102 | 0.05 | 0.7378 | 1.8087 | 1.5265 | 2 | 2 |
| WCM365 | 6 | 32146358 | 34835294 | −0.411 | 0.0193 | 1.6738 | 0.3532 | 2 | 0 |
| WCM365 | 6 | 34838640 | 57190745 | −0.056 | 0.5832 | 1.896 | 1.1004 | 2 | 1 |
| WCM365 | 6 | 57244672 | 67859571 | 0.2333 | 0.994 | 2.3584 | 1.6251 | 2 | 2 |
| WCM365 | 6 | 69345889 | 76624584 | 0.0119 | 0.6828 | 1.7264 | 1.4842 | 2 | 1 |
| WCM365 | 6 | 76625632 | 84935986 | 0.4609 | 1.2962 | 3.6514 | 1.2601 | 4 | 1 |
| WCM365 | 6 | 85398259 | 170892873 | −0.1294 | 0.4731 | 1.3881 | 1.3881 | 1 | 1 |
| WCM365 | 7 | 193222 | 933585 | 0.1209 | 0.8385 | 1.8635 | 1.7128 | 2 | 2 |
| WCM365 | 7 | 935032 | 83758396 | 0.1955 | 0.9422 | 1.9214 | 1.9214 | 2 | 2 |
| WCM365 | 7 | 83764092 | 158937161 | 0.114 | 0.8287 | 1.8061 | 1.7461 | 2 | 2 |
| WCM365 | 8 | 163470 | 35406923 | −0.7423 | −0.6061 | 1.3157 | −0.0018 | 1 | 0 |
| WCM365 | 8 | 35425695 | 56652020 | −0.2156 | 0.3397 | 2.5588 | −0.0278 | 3 | 0 |
| WCM365 | 8 | 56652748 | 90777609 | 0.4966 | 1.3423 | 2.7898 | 2.2813 | 3 | 2 |
| WCM365 | 8 | 90782014 | 105601105 | 0.7934 | 1.7137 | 4.5013 | 2.0587 | 5 | 2 |
| WCM365 | 8 | 106331156 | 125498283 | 0.5246 | 1.3781 | 2.935 | 2.2637 | 3 | 2 |
| WCM365 | 8 | 125498510 | 132052217 | 0.9457 | 1.8976 | 5.1545 | 2.2975 | 5 | 2 |
| WCM365 | 8 | 132052526 | 144621084 | 0.4981 | 1.3442 | 2.5814 | 2.4966 | 3 | 2 |
| WCM365 | 8 | 144621355 | 146279416 | 0.1303 | 0.8516 | 1.8349 | 1.7742 | 2 | 2 |
| WCM365 | 9 | 116924 | 9425617 | −0.671 | −0.4601 | 1.4685 | −0.0146 | 1 | 0 |
| WCM365 | 9 | 9766889 | 21482089 | −0.2144 | 0.3417 | 2.4899 | 0.0445 | 2 | 0 |
| WCM365 | 9 | 21512197 | 21967669 | 1.4355 | 2.4659 | NA | NA | NA | NA |
| WCM365 | 9 | 21968126 | 22452130 | −0.5336 | −0.1977 | NA | NA | NA | NA |
| WCM365 | 9 | 23690357 | 34729468 | 1.0981 | 2.0778 | 5.3741 | 3.0693 | 5 | 3 |
| WCM365 | 9 | 34832762 | 38604943 | 0.6683 | 1.5595 | 2.9476 | 2.9476 | 3 | 3 |
| WCM365 | 9 | 38607419 | 141107525 | 0.0514 | 0.7399 | 1.6701 | 1.6701 | 2 | 2 |
| WCM365 | X | 2700156 | 44870323 | 0.1964 | 0.9435 | NA | NA | NA | NA |
| WCM365 | X | 44879891 | 46584900 | 0.848 | 1.78 | NA | NA | NA | NA |
| WCM365 | X | 46696611 | 64628427 | 0.4003 | 1.2172 | NA | NA | NA | NA |
| WCM365 | X | 64708632 | 65835822 | 1.234 | 2.2358 | NA | NA | NA | NA |
| WCM365 | X | 66765005 | 68748959 | 3.2585 | 4.4186 | NA | NA | NA | NA |
| WCM365 | X | 68749585 | 154493598 | 0.1486 | 0.8773 | NA | NA | NA | NA |
| WCM365_Z2_1 | 1 | 721450 | 29651762 | −0.569 | 0.0142 | 2.011 | 0.0088 | 2 | 0 |
| WCM365_Z2_1 | 1 | 29652118 | 32793189 | −0.2981 | 0.3564 | NA | NA | NA | NA |
| WCM365_Z2_1 | 1 | 32794689 | 120612034 | −0.5851 | −0.0066 | 1.9854 | 0.0054 | 2 | 0 |
| WCM365_Z2_1 | 1 | 144014068 | 225606973 | 0.4754 | 1.2633 | 2.8457 | 1.955 | 3 | 2 |
| WCM365_Z2_1 | 1 | 225609862 | 228786362 | −0.1668 | 0.5168 | 2.8575 | 0.0039 | 3 | 0 |
| WCM365_Z2_1 | 1 | 228871682 | 230933950 | 1.2081 | 2.0658 | NA | NA | NA | NA |
| WCM365_Z2_1 | 1 | 230937343 | 235424048 | −0.2235 | 0.4479 | 2.7674 | −0.0393 | 3 | 0 |
| WCM365_Z2_1 | 1 | 235498577 | 235590541 | 1.0008 | 1.8425 | NA | NA | NA | NA |
| WCM365_Z2_1 | 1 | 235594065 | 241953929 | 1.2301 | 2.0893 | 5.8583 | 2.6523 | 6 | 3 |
| WCM365_Z2_1 | 1 | 241958560 | 249212401 | 0.8613 | 1.6907 | 3.7818 | 2.6744 | 4 | 3 |
| WCM365_Z2_1 | 10 | 255883 | 34985257 | −0.6617 | −0.107 | 1.8612 | −0.0042 | 2 | 0 |
| WCM365_Z2_1 | 10 | 35103898 | 35930068 | 0.66 | 1.4692 | NA | NA | NA | NA |
| WCM365_Z2_1 | 10 | 35930331 | 49440168 | 0.1008 | 0.8345 | 1.8852 | 1.6812 | 2 | 2 |
| WCM365_Z2_1 | 10 | 49444555 | 54528022 | 0.335 | 1.1044 | 2.2651 | 2.0349 | 2 | 2 |
| WCM365_Z2_1 | 10 | 54528252 | 67829154 | 0.0883 | 0.8198 | 1.8333 | 1.697 | 2 | 2 |
| WCM365_Z2_1 | 10 | 67862924 | 84118519 | 0.5516 | 1.3486 | 3.0674 | 2.026 | 3 | 2 |
| WCM365_Z2_1 | 10 | 84498367 | 135440105 | −0.5907 | −0.0139 | 1.9766 | 0.0042 | 2 | 0 |
| WCM365_Z2_1 | 11 | 193146 | 107578641 | 0.2123 | 0.9638 | 1.9797 | 1.9212 | 2 | 2 |
| WCM365_Z2_1 | 11 | 107647302 | 111430956 | −0.2405 | 0.4271 | NA | NA | NA | NA |
| WCM365_Z2_1 | 11 | 111431184 | 125351473 | −0.5496 | 0.0393 | 2.0396 | 0.0156 | 2 | 0 |
| WCM365_Z2_1 | 11 | 125359503 | 134241680 | −0.1095 | 0.5857 | 1.9543 | 1.0473 | 2 | 1 |
| WCM365_Z2_1 | 11 | 134243650 | 134856603 | 0.0034 | 0.72 | NA | NA | NA | NA |
| WCM365_Z2_1 | 12 | 148644 | 40715834 | −0.1331 | 0.5573 | 1.9053 | 1.0378 | 2 | 1 |
| WCM365_Z2_1 | 12 | 40716260 | 49168253 | −0.0369 | 0.6723 | 1.8547 | 1.3325 | 2 | 1 |
| WCM365_Z2_1 | 12 | 49168481 | 123035889 | −0.1294 | 0.5618 | 1.9837 | 0.9685 | 2 | 1 |
| WCM365_Z2_1 | 12 | 123036236 | 133810935 | 0.1066 | 0.8412 | 2.6485 | 0.9345 | 3 | 1 |
| WCM365_Z2_1 | 13 | 19600515 | 80117742 | −0.151 | 0.5358 | 1.9096 | 0.9899 | 2 | 1 |
| WCM365_Z2_1 | 13 | 80125186 | 115089411 | −0.5973 | −0.0226 | 1.9584 | 0.0105 | 2 | 0 |
| WCM365_Z2_1 | 14 | 19377692 | 23888815 | 0.1989 | 0.9484 | 1.952 | 1.9074 | 2 | 2 |
| WCM365_Z2_1 | 14 | 23889048 | 107283183 | −0.1227 | 0.5699 | 1.9908 | 0.978 | 2 | 1 |
| WCM365_Z2_1 | 15 | 20170044 | 22489992 | −0.8809 | −0.4045 | 1.5735 | −0.0626 | 2 | 0 |
| WCM365_Z2_1 | 15 | 22833664 | 65792946 | −0.5863 | −0.0082 | 1.9835 | 0.0052 | 2 | 0 |
| WCM365_Z2_1 | 15 | 65804678 | 66857697 | −0.0904 | 0.6086 | NA | NA | NA | NA |
| WCM365_Z2_1 | 15 | 66976341 | 69684016 | −1.2345 | −0.9259 | NA | NA | NA | NA |
| WCM365_Z2_1 | 15 | 69689537 | 102506276 | −0.1289 | 0.5624 | 1.9615 | 0.9921 | 2 | 1 |
| WCM365_Z2_1 | 16 | 67604 | 51185127 | 0.2365 | 0.9916 | 2.0067 | 1.9701 | 2 | 2 |
| WCM365_Z2_1 | 16 | 52060303 | 90233297 | −0.5822 | −0.0029 | 1.9887 | 0.0073 | 2 | 0 |
| WCM365_Z2_1 | 17 | 131595 | 6719251 | 0.3544 | 1.1264 | 4.3647 | 0.0016 | 4 | 0 |
| WCM365_Z2_1 | 17 | 6722644 | 10267770 | −0.4426 | 0.176 | 2.2711 | −0.0116 | 2 | 0 |
| WCM365_Z2_1 | 17 | 10293875 | 29902304 | 0.3285 | 1.0969 | 2.1926 | 2.0854 | 2 | 2 |
| WCM365_Z2_1 | 17 | 29902620 | 41277201 | −0.0859 | 0.6139 | 1.9493 | 1.1116 | 2 | 1 |
| WCM365_Z2_1 | 17 | 41290786 | 42478676 | −1.2331 | −0.9237 | 1.0061 | 0.0482 | 1 | 0 |
| WCM365_Z2_1 | 17 | 42501781 | 49297776 | −0.5807 | −9.00E−04 | 1.0344 | 0.9644 | 1 | 1 |
| WCM365_Z2_1 | 17 | 49338241 | 59770797 | −1.2554 | −0.9587 | 1.0024 | 0.0266 | 1 | 0 |
| WCM365_Z2_1 | 17 | 59793382 | 63010673 | −0.5706 | 0.0122 | 1.1457 | 0.8712 | 1 | 1 |
| WCM365_Z2_1 | 17 | 63049623 | 81083561 | 0.2496 | 1.0068 | 2.965 | 1.0539 | 3 | 1 |
| WCM365_Z2_1 | 18 | 48220 | 2674028 | 0.8911 | 1.7232 | NA | NA | NA | NA |
| WCM365_Z2_1 | 18 | 2688626 | 3603996 | 1.5814 | 2.4624 | NA | NA | NA | NA |
| WCM365_Z2_1 | 18 | 3708471 | 15006148 | 0.7621 | 1.582 | 3.8631 | 2.1246 | 4 | 2 |
| WCM365_Z2_1 | 18 | 18531342 | 24081088 | −0.1336 | 0.5568 | 1.8337 | 1.1082 | 2 | 1 |
| WCM365_Z2_1 | 18 | 24126843 | 70416282 | −0.5977 | −0.0231 | 1.9617 | 0.0066 | 2 | 0 |
| WCM365_Z2_1 | 18 | 70417525 | 73139433 | −0.1752 | 0.5065 | 1.9244 | 0.9169 | 2 | 1 |
| WCM365_Z2_1 | 18 | 74074452 | 74980800 | 2.3029 | 3.2142 | NA | NA | NA | NA |
| WCM365_Z2_1 | 18 | 76856549 | 78005199 | −0.2696 | 0.3915 | NA | NA | NA | NA |
| WCM365_Z2_1 | 19 | 281508 | 3595923 | −0.5379 | 0.0545 | 2.0842 | −0.0072 | 2 | 0 |
| WCM365_Z2_1 | 19 | 3600000 | 4097323 | 0.257 | 1.0152 | 2.0746 | 1.9679 | 2 | 2 |
| WCM365_Z2_1 | 19 | 4099350 | 5262989 | 0.7544 | 1.5734 | 3.88 | 2.0723 | 4 | 2 |
| WCM365_Z2_1 | 19 | 5265089 | 9971456 | 0.5304 | 1.3249 | 2.9314 | 2.079 | 3 | 2 |
| WCM365_Z2_1 | 19 | 10071227 | 36967452 | −0.1161 | 0.5777 | 1.969 | 1.016 | 2 | 1 |
| WCM365_Z2_1 | 19 | 36980642 | 39218979 | 0.4979 | 1.2885 | 2.769 | 2.1165 | 3 | 2 |
| WCM365_Z2_1 | 19 | 39219780 | 39989894 | −0.0862 | 0.6136 | 3.0681 | −0.0079 | 3 | 0 |
| WCM365_Z2_1 | 19 | 39991293 | 43269732 | −1.2455 | −0.9432 | 1.0048 | 0.0354 | 1 | 0 |
| WCM365_Z2_1 | 19 | 43341536 | 43530527 | −2.3115 | −3.436 | NA | NA | NA | NA |
| WCM365_Z2_1 | 19 | 43568732 | 43883177 | −1.2802 | −0.9981 | NA | NA | NA | NA |
| WCM365_Z2_1 | 19 | 43910667 | 51381789 | −0.113 | 0.5815 | 2.9827 | 0.0101 | 3 | 0 |
| WCM365_Z2_1 | 19 | 51385856 | 53383944 | 0.5054 | 1.297 | 2.9808 | 1.9336 | 3 | 2 |
| WCM365_Z2_1 | 19 | 53384185 | 57640301 | 1.2757 | 2.138 | 6.8222 | 1.9809 | 7 | 2 |
| WCM365_Z2_1 | 19 | 57640593 | 58806837 | 2.1821 | 3.0894 | 15.0401 | 1.9832 | 15 | 2 |
| WCM365_Z2_1 | 19 | 58846414 | 59083923 | −0.54 | 0.0517 | NA | NA | NA | NA |
| WCM365_Z2_1 | 2 | 41613 | 80529766 | −0.1008 | 0.5962 | 1.948 | 1.0754 | 2 | 1 |
| WCM365_Z2_1 | 2 | 80530029 | 80773128 | 0.3849 | 1.1611 | NA | NA | NA | NA |
| WCM365_Z2_1 | 2 | 80782965 | 97166614 | 0.2263 | 0.98 | 1.9974 | 1.9475 | 2 | 2 |
| WCM365_Z2_1 | 2 | 97171003 | 155306936 | 0.4743 | 1.262 | 2.8056 | 1.9911 | 3 | 2 |
| WCM365_Z2_1 | 2 | 155555473 | 242946604 | −0.1593 | 0.5257 | 1.9786 | 0.9008 | 2 | 1 |
| WCM365_Z2_1 | 20 | 68363 | 26188896 | −0.6072 | −0.0354 | 1.9456 | 0.0059 | 2 | 0 |
| WCM365_Z2_1 | 20 | 29632632 | 62904848 | 0.2404 | 0.9962 | 2.0062 | 1.9832 | 2 | 2 |
| WCM365_Z2_1 | 21 | 9907318 | 39822803 | −0.1503 | 0.5366 | 1.8862 | 1.015 | 2 | 1 |
| WCM365_Z2_1 | 21 | 39947620 | 42870095 | −0.6154 | −0.0461 | 1.9975 | −0.0605 | 2 | 0 |
| WCM365_Z2_1 | 21 | 43133055 | 48084277 | −0.0958 | 0.6022 | 1.9485 | 1.0874 | 2 | 1 |
| WCM365_Z2_1 | 22 | 16287495 | 41676968 | −0.5662 | 0.0178 | 2.0194 | 0.0055 | 2 | 0 |
| WCM365_Z2_1 | 22 | 41716687 | 42828268 | −0.0652 | 0.6387 | 3.1156 | −0.0018 | 3 | 0 |
| WCM365_Z2_1 | 22 | 42896695 | 51215152 | −0.5661 | 0.018 | 2.017 | 0.0081 | 2 | 0 |
| WCM365_Z2_1 | 3 | 239350 | 71179680 | −0.5562 | 0.0308 | 2.0342 | 0.0089 | 2 | 0 |
| WCM365_Z2_1 | 3 | 71247213 | 197894720 | −0.1376 | 0.5519 | 1.9655 | 0.9666 | 2 | 1 |
| WCM365_Z2_1 | 4 | 59339 | 2464581 | −0.5126 | 0.0869 | 2.1194 | 0.0048 | 2 | 0 |
| WCM365_Z2_1 | 4 | 2498826 | 90874552 | 0.2958 | 1.0597 | 2.1178 | 2.0511 | 2 | 2 |
| WCM365_Z2_1 | 4 | 91229611 | 93218685 | −0.2963 | 0.3587 | NA | NA | NA | NA |
| WCM365_Z2_1 | 4 | 93221978 | 164533963 | −0.1787 | 0.5023 | 1.8401 | 0.9928 | 2 | 1 |
| WCM365_Z2_1 | 4 | 164534482 | 190426502 | −0.356 | 0.2847 | 1.4472 | 0.9891 | 1 | 1 |
| WCM365_Z2_1 | 5 | 140644 | 52405546 | −0.0111 | 0.7029 | 1.9611 | 1.2945 | 2 | 1 |
| WCM365_Z2_1 | 5 | 52778774 | 64625289 | −0.616 | −0.047 | 1.0351 | 0.9008 | 1 | 1 |
| WCM365_Z2_1 | 5 | 64747418 | 68411126 | −0.1477 | 0.5397 | NA | NA | NA | NA |
| WCM365_Z2_1 | 5 | 68411965 | 131409786 | −0.6148 | −0.0453 | 0.9868 | 0.9513 | 1 | 1 |
| WCM365_Z2_1 | 5 | 131410607 | 133304151 | −1.2787 | −0.9958 | NA | NA | NA | NA |
| WCM365_Z2_1 | 5 | 133308513 | 143853561 | −0.5751 | 0.0064 | 1.1673 | 0.8415 | 1 | 1 |
| WCM365_Z2_1 | 5 | 145176022 | 180687808 | 0.2061 | 0.9566 | 2.9284 | 0.9532 | 3 | 1 |
| WCM365_Z2_1 | 6 | 203552 | 367352 | −0.1833 | 0.4968 | NA | NA | NA | NA |
| WCM365_Z2_1 | 6 | 393256 | 10557344 | −1.2868 | −1.0088 | 0.9561 | 0.0379 | 1 | 0 |
| WCM365_Z2_1 | 6 | 10557539 | 18572108 | −0.183 | 0.4971 | 1.9227 | 0.9001 | 2 | 1 |
| WCM365_Z2_1 | 6 | 19838198 | 25509925 | 1.1923 | 2.0488 | 6.4536 | 1.8219 | 6 | 2 |
| WCM365_Z2_1 | 6 | 25510801 | 30782303 | 0.6685 | 1.4786 | 3.6323 | 1.9414 | 4 | 2 |
| WCM365_Z2_1 | 6 | 30858815 | 32139228 | 0.2417 | 0.9977 | 2.107 | 1.8866 | 2 | 2 |
| WCM365_Z2_1 | 6 | 32159158 | 32796751 | −0.1903 | 0.4883 | NA | NA | NA | NA |
| WCM365_Z2_1 | 6 | 32800412 | 33756205 | −0.554 | 0.0336 | NA | NA | NA | NA |
| WCM365_Z2_1 | 6 | 33763331 | 36068017 | −0.1319 | 0.5589 | NA | NA | NA | NA |
| WCM365_Z2_1 | 6 | 36075351 | 54254674 | −0.5851 | −0.0067 | 1.0407 | 0.9501 | 1 | 1 |
| WCM365_Z2_1 | 6 | 54735316 | 76751755 | −0.1499 | 0.5371 | 1.8413 | 1.0609 | 2 | 1 |
| WCM365_Z2_1 | 6 | 76782138 | 84936092 | 0.4404 | 1.2238 | 3.6712 | 1 | 4 | 1 |
| WCM365_Z2_1 | 6 | 85398382 | 170893424 | −0.1531 | 0.5333 | 1.9012 | 0.9933 | 2 | 1 |
| WCM365_Z2_1 | 7 | 193222 | 71743764 | 0.1946 | 0.9434 | 1.943 | 1.903 | 2 | 2 |
| WCM365_Z2_1 | 7 | 71868350 | 77045605 | 1.6884 | 2.5749 | 9.8504 | 2.0665 | 10 | 2 |
| WCM365_Z2_1 | 7 | 77166921 | 158937046 | 0.2233 | 0.9765 | 2.0017 | 1.9337 | 2 | 2 |
| WCM365_Z2_1 | 8 | 190900 | 32621527 | −0.596 | −0.0209 | 1.9502 | 0.0211 | 2 | 0 |
| WCM365_Z2_1 | 8 | 33230198 | 56436496 | 0.2127 | 0.9643 | 3.8769 | 0.0253 | 4 | 0 |
| WCM365_Z2_1 | 8 | 56656130 | 64099757 | 0.5648 | 1.3634 | NA | NA | NA | NA |
| WCM365_Z2_1 | 8 | 64099940 | 113841912 | 1.0951 | 1.9443 | 5.6236 | 2.0735 | 6 | 2 |
| WCM365_Z2_1 | 8 | 113871381 | 144400207 | 1.2852 | 2.1482 | 5.7386 | 3.1268 | 6 | 3 |
| WCM365_Z2_1 | 8 | 144403485 | 146279481 | 0.556 | 1.3536 | 3.1235 | 1.9875 | 3 | 2 |
| WCM365_Z2_1 | 9 | 118113 | 8733796 | −1.332 | −1.0818 | 0.9567 | −0.0118 | 1 | 0 |
| WCM365_Z2_1 | 9 | 12694114 | 21481664 | −0.5857 | −0.0074 | 1.991 | −0.0012 | 2 | 0 |
| WCM365_Z2_1 | 9 | 21512150 | 21861975 | 1.9144 | 2.8113 | NA | NA | NA | NA |
| WCM365_Z2_1 | 9 | 21931138 | 22451832 | −0.8298 | −0.3337 | NA | NA | NA | NA |
| WCM365_Z2_1 | 9 | 23692622 | 34723744 | 1.0947 | 1.9439 | 5.7465 | 1.9486 | 6 | 2 |
| WCM365_Z2_1 | 9 | 34723935 | 43884984 | 0.5041 | 1.2955 | 2.9999 | 1.9095 | 3 | 2 |
| WCM365_Z2_1 | 9 | 44870094 | 141111414 | −0.1317 | 0.5591 | 1.9216 | 1.025 | 2 | 1 |
| WCM365_Z2_1 | X | 2712619 | 44833908 | −0.643 | −0.0824 | NA | NA | NA | NA |
| WCM365_Z2_1 | X | 44879891 | 51151372 | 1.7957 | 2.6874 | NA | NA | NA | NA |
| WCM365_Z2_1 | X | 51644748 | 65835798 | 0.7194 | 1.5349 | NA | NA | NA | NA |
| WCM365_Z2_1 | X | 66765060 | 67431965 | 5.5436 | 6.4959 | NA | NA | NA | NA |
| WCM365_Z2_1 | X | 67454402 | 154509348 | 0.1539 | 0.8963 | NA | NA | NA | NA |
| WCM366 | 1 | 14930 | 249213961 | 0.0368 | 0.1832 | 1.1354 | 1.1354 | 1 | 1 |
| WCM366 | 10 | 225979 | 135477891 | 0.0288 | −0.0056 | 0.9961 | 0.9961 | 1 | 1 |
| WCM366 | 11 | 193112 | 134605916 | 0.0348 | 0.1386 | 1.2012 | 1.0004 | 1 | 1 |
| WCM366 | 12 | 148560 | 133810703 | 0.0173 | −0.3263 | 0.7976 | 0.7976 | 1 | 1 |
| WCM366 | 13 | 19041979 | 115089411 | 0.022 | −0.185 | 0.9543 | 0.805 | 1 | 1 |
| WCM366 | 14 | 19110178 | 107283269 | 0.0257 | −0.084 | 0.9434 | 0.9434 | 1 | 1 |
| WCM366 | 15 | 20083896 | 102506240 | 0.041 | 0.2741 | 1.2093 | 1.2093 | 1 | 1 |
| WCM366 | 16 | 64423 | 90260461 | 0.02 | −0.2438 | 0.919 | 0.77 | 1 | 1 |
| WCM366 | 17 | 131595 | 81187906 | 0.0176 | −0.3152 | 0.8038 | 0.8038 | 1 | 1 |
| WCM366 | 18 | 47610 | 78005135 | 0.0269 | −0.0527 | 0.9641 | 0.9641 | 1 | 1 |
| WCM366 | 19 | 105080 | 59093696 | −0.0334 | NA | −0.0579 | −0.0579 | 0 | 0 |
| WCM366 | 2 | 41463 | 243052762 | 0.0164 | −0.3539 | 0.7824 | 0.7824 | 1 | 1 |
| WCM366 | 20 | 68299 | 62934617 | 0.0537 | 0.5181 | 1.4321 | 1.4321 | 1 | 1 |
| WCM366 | 21 | 9590331 | 48084167 | 0.0275 | −0.0388 | 1.0484 | 0.8986 | 1 | 1 |
| WCM366 | 22 | 16159079 | 51219089 | 0.066 | 0.7224 | 1.6756 | 1.6243 | 2 | 2 |
| WCM366 | 3 | 239472 | 197896697 | 0.0288 | −0.0048 | 0.9967 | 0.9967 | 1 | 1 |
| WCM366 | 4 | 53334 | 190903980 | 0.0121 | −0.4948 | 0.7097 | 0.7097 | 1 | 1 |
| WCM366 | 5 | 90751 | 180687861 | 0.0079 | −0.6525 | 0.6362 | 0.6362 | 1 | 1 |
| WCM366 | 6 | 203509 | 170892848 | 0.0191 | −0.2695 | 0.8296 | 0.8296 | 1 | 1 |
| WCM366 | 7 | 193205 | 158937142 | 0.0103 | −0.5608 | 0.9247 | 0.4311 | 1 | 0 |
| WCM366 | 8 | 163457 | 146279508 | 0.0215 | −0.2004 | 0.8703 | 0.8703 | 1 | 1 |
| WCM366 | 9 | 14822 | 141107582 | 0.0392 | 0.2359 | 1.1776 | 1.1776 | 1 | 1 |
| WCM366 | X | 2700119 | 154509348 | −0.0997 | NA | NA | NA | NA | NA |
| WCM366_X1_1 | 1 | 721476 | 91447889 | 0.0667 | −0.0108 | 0.9999 | 0.9853 | 1 | 1 |
| WCM366_X1_1 | 1 | 91731640 | 99470218 | −0.4962 | −0.9436 | 1.0607 | −0.0209 | 1 | 0 |
| WCM366_X1_1 | 1 | 99730124 | 155018619 | 0.0644 | −0.0142 | 1.0968 | 0.8836 | 1 | 1 |
| WCM366_X1_1 | 1 | 155018860 | 157811484 | −0.5509 | −1.0498 | 0.9608 | 0.0053 | 1 | 0 |
| WCM366_X1_1 | 1 | 157963503 | 249212244 | 0.0603 | −0.0202 | 1.005 | 0.9672 | 1 | 1 |
| WCM366_X1_1 | 10 | 255873 | 50603453 | 0.0721 | −0.0028 | 1.0259 | 0.9701 | 1 | 1 |
| WCM366_X1_1 | 10 | 50666859 | 88445446 | −0.5388 | −1.026 | 0.9854 | −0.0032 | 1 | 0 |
| WCM366_X1_1 | 10 | 88446946 | 90537781 | −1.4777 | −5.0095 | NA | NA | NA | NA |
| WCM366_X1_1 | 10 | 90565762 | 135440108 | −0.5127 | −0.9753 | 1.0031 | 0.0141 | 1 | 0 |
| WCM366_X1_1 | 11 | 193112 | 134856563 | 0.0631 | −0.016 | 0.9992 | 0.9788 | 1 | 1 |
| WCM366_X1_1 | 12 | 148675 | 133810703 | 0.0713 | −0.0039 | 1.009 | 0.9855 | 1 | 1 |
| WCM366_X1_1 | 13 | 19600515 | 39622060 | 0.128 | 0.0787 | 1.0835 | 1.0286 | 1 | 1 |
| WCM366_X1_1 | 13 | 39918139 | 109562474 | −0.5388 | −1.0261 | 1.0401 | −0.058 | 1 | 0 |
| WCM366_X1_1 | 13 | 109610052 | 115089411 | 0.0812 | 0.0106 | 1.0458 | 0.969 | 1 | 1 |
| WCM366_X1_1 | 14 | 19377710 | 55655757 | 0.0479 | −0.0386 | 0.9881 | 0.9592 | 1 | 1 |
| WCM366_X1_1 | 14 | 55817136 | 58678089 | −0.5971 | −1.1424 | NA | NA | NA | NA |
| WCM366_X1_1 | 14 | 58680352 | 74083896 | 0.0625 | −0.017 | 1.0218 | 0.9547 | 1 | 1 |
| WCM366_X1_1 | 14 | 74085999 | 93182528 | −0.5276 | −1.0042 | 0.9977 | −6.00E−04 | 1 | 0 |
| WCM366_X1_1 | 14 | 93183771 | 107283203 | 0.0741 | 2.00E−04 | 1.0163 | 0.9839 | 1 | 1 |
| WCM366_X1_1 | 15 | 20169955 | 25496043 | −0.4307 | −0.8215 | 0.9939 | 0.1378 | 1 | 0 |
| WCM366_X1_1 | 15 | 25514932 | 102506276 | 0.0573 | −0.0246 | 0.9991 | 0.9671 | 1 | 1 |
| WCM366_X1_1 | 16 | 66953 | 90161103 | 0.0769 | 0.0043 | 1.0148 | 0.9912 | 1 | 1 |
| WCM366_X1_1 | 17 | 131595 | 5424198 | 0.0799 | 0.0086 | 1.0193 | 0.9927 | 1 | 1 |
| WCM366_X1_1 | 17 | 5424989 | 8224276 | −0.544 | −1.0361 | 1.0223 | −0.0471 | 1 | 0 |
| WCM366_X1_1 | 17 | 8243498 | 16351411 | 0.0876 | 0.0199 | 1.0451 | 0.9827 | 1 | 1 |
| WCM366_X1_1 | 17 | 16365616 | 16665658 | 0.5029 | 0.5937 | NA | NA | NA | NA |
| WCM366_X1_1 | 17 | 16734836 | 17962196 | 0.053 | −0.031 | 2.0151 | −0.0575 | 2 | 0 |
| WCM366_X1_1 | 17 | 17965234 | 21191325 | 0.4549 | 0.5304 | 1.9575 | 0.9311 | 2 | 1 |
| WCM366_X1_1 | 17 | 21194843 | 31267828 | 0.057 | −0.025 | 1.0468 | 0.9189 | 1 | 1 |
| WCM366_X1_1 | 17 | 31318958 | 34122377 | −0.5313 | −1.0114 | 0.9504 | 0.0417 | 1 | 0 |
| WCM366_X1_1 | 17 | 34182122 | 81083419 | 0.0745 | 8.00E−04 | 1.0123 | 0.9888 | 1 | 1 |
| WCM366_X1_1 | 18 | 48209 | 21492808 | 0.0687 | −0.0078 | 1.0239 | 0.9653 | 1 | 1 |
| WCM366_X1_1 | 18 | 21494398 | 30351030 | −0.5641 | −1.0759 | 0.926 | 0.0227 | 1 | 0 |
| WCM366_X1_1 | 18 | 30353290 | 56587778 | 0.0598 | −0.021 | 0.9929 | 0.9783 | 1 | 1 |
| WCM366_X1_1 | 18 | 56601821 | 78005214 | −0.5358 | −1.0201 | 1.0163 | −0.0302 | 1 | 0 |
| WCM366_X1_1 | 19 | 281503 | 8212353 | −0.523 | −0.9953 | 0.9926 | 0.0107 | 1 | 0 |
| WCM366_X1_1 | 19 | 8316022 | 47551844 | 0.064 | −0.0148 | 1.0073 | 0.9723 | 1 | 1 |
| WCM366_X1_1 | 19 | 47569690 | 48364615 | −0.5442 | −1.0367 | NA | NA | NA | NA |
| WCM366_X1_1 | 19 | 48374796 | 59083968 | 0.0716 | −0.0036 | 1.0107 | 0.9843 | 1 | 1 |
| WCM366_X1_1 | 2 | 41623 | 242946607 | 0.0695 | −0.0067 | 1.0042 | 0.9866 | 1 | 1 |
| WCM366_X1_1 | 20 | 68373 | 6100088 | −0.5292 | −1.0072 | 1.0145 | −0.0195 | 1 | 0 |
| WCM366_X1_1 | 20 | 6750903 | 62384099 | 0.0664 | −0.0112 | 1.0069 | 0.9777 | 1 | 1 |
| WCM366_X1_1 | 20 | 62407104 | 62904845 | −0.5089 | −0.968 | 1.0208 | 0.0017 | 1 | 0 |
| WCM366_X1_1 | 21 | 9907297 | 39775468 | 0.0682 | −0.0086 | 1.0277 | 0.9604 | 1 | 1 |
| WCM366_X1_1 | 21 | 39795398 | 42870081 | −0.512 | −0.974 | 0.9774 | 0.0408 | 1 | 0 |
| WCM366_X1_1 | 21 | 43133077 | 48084207 | 0.0737 | −4.00E−04 | 1.0306 | 0.9688 | 1 | 1 |
| WCM366_X1_1 | 22 | 17063485 | 51215124 | 0.0709 | −0.0046 | 1.0174 | 0.9763 | 1 | 1 |
| WCM366_X1_1 | 3 | 239347 | 45077391 | 0.0816 | 0.0111 | 1.0181 | 0.9974 | 1 | 1 |
| WCM366_X1_1 | 3 | 45127226 | 49828287 | −0.5224 | −0.9941 | 1.0253 | −0.0212 | 1 | 0 |
| WCM366_X1_1 | 3 | 49828879 | 89521694 | 0.0658 | −0.012 | 1.0136 | 0.9698 | 1 | 1 |
| WCM366_X1_1 | 3 | 89528644 | 102196386 | −0.5023 | −0.9553 | 0.9961 | 0.0354 | 1 | 0 |
| WCM366_X1_1 | 3 | 105086318 | 137889032 | 0.064 | −0.0147 | 1.0249 | 0.9548 | 1 | 1 |
| WCM366_X1_1 | 3 | 137890526 | 139181115 | 0.5033 | 0.5943 | NA | NA | NA | NA |
| WCM366_X1_1 | 3 | 139195237 | 189586486 | 0.0863 | 0.0181 | 1.0275 | 0.9978 | 1 | 1 |
| WCM366_X1_1 | 3 | 189587169 | 197894720 | −0.5576 | −1.063 | 0.9608 | −0.0036 | 1 | 0 |
| WCM366_X1_1 | 4 | 59376 | 190426471 | 0.0714 | −0.0038 | 1.0091 | 0.9856 | 1 | 1 |
| WCM366_X1_1 | 5 | 140526 | 180687800 | 0.0742 | 3.00E−04 | 1.0119 | 0.9884 | 1 | 1 |
| WCM366_X1_1 | 6 | 203535 | 72011510 | 0.0638 | −0.015 | 1.0013 | 0.978 | 1 | 1 |
| WCM366_X1_1 | 6 | 72086672 | 129465150 | −0.2462 | −0.4995 | 0.9888 | 0.4258 | 1 | 0 |
| WCM366_X1_1 | 6 | 129475727 | 170893415 | 0.0728 | −0.0017 | 1.015 | 0.9827 | 1 | 1 |
| WCM366_X1_1 | 7 | 193265 | 158937400 | 0.0666 | −0.0109 | 1.0042 | 0.9808 | 1 | 1 |
| WCM366_X1_1 | 8 | 190906 | 9912088 | −0.5486 | −1.0453 | 0.9755 | −0.0064 | 1 | 0 |
| WCM366_X1_1 | 8 | 10065365 | 10623169 | −1.4484 | −4.5969 | 0.0649 | 0.0178 | 0 | 0 |
| WCM366_X1_1 | 8 | 10645331 | 25902325 | −0.521 | −0.9914 | 1.018 | −0.012 | 1 | 0 |
| WCM366_X1_1 | 8 | 26149344 | 27402010 | −1.6289 | NA | NA | NA | NA | NA |
| WCM366_X1_1 | 8 | 27455988 | 39525545 | −0.4997 | −0.9504 | 1.0303 | 0.0047 | 1 | 0 |
| WCM366_X1_1 | 8 | 39534982 | 40683184 | −1.596 | NA | NA | NA | NA | NA |
| WCM366_X1_1 | 8 | 41122763 | 67817527 | −0.5399 | −1.0281 | 1.0755 | −0.0949 | 1 | 0 |
| WCM366_X1_1 | 8 | 67831389 | 146279507 | 0.0668 | −0.0106 | 0.9927 | 0.9927 | 1 | 1 |
| WCM366_X1_1 | 9 | 17084 | 916823 | 0.0539 | −0.0297 | NA | NA | NA | NA |
| WCM366_X1_1 | 9 | 968037 | 6255988 | −0.5027 | −0.9561 | 1.0597 | −0.0288 | 1 | 0 |
| WCM366_X1_1 | 9 | 6328813 | 16020595 | 0.0467 | −0.0403 | 1.0388 | 0.9061 | 1 | 1 |
| WCM366_X1_1 | 9 | 16215239 | 19050060 | −0.6584 | −1.2704 | NA | NA | NA | NA |
| WCM366_X1_1 | 9 | 19050259 | 79635870 | 0.071 | −0.0045 | 1.0233 | 0.9705 | 1 | 1 |
| WCM366_X1_1 | 9 | 79792635 | 89771644 | −0.4696 | −0.8935 | NA | NA | NA | NA |
| WCM366_X1_1 | 9 | 90062823 | 113341692 | 0.0944 | 0.0298 | 1.0388 | 1.003 | 1 | 1 |
| WCM366_X1_1 | 9 | 113341804 | 119249760 | −0.5307 | −1.0102 | 0.9723 | 0.0207 | 1 | 0 |
| WCM366_X1_1 | 9 | 119380680 | 141111414 | 0.0792 | 0.0077 | 1.0186 | 0.9921 | 1 | 1 |
| WCM366_X1_1 | X | 2712639 | 154509348 | 0.0213 | −0.0782 | NA | NA | NA | NA |
| WCM369 | 1 | 14907 | 249213420 | 0.0381 | 0.1251 | 1.0906 | 1.0906 | 1 | 1 |
| WCM369 | 10 | 225982 | 135438959 | 0.0299 | 0.0133 | 1.0092 | 1.0092 | 1 | 1 |
| WCM369 | 11 | 193096 | 62285788 | 0.0444 | 0.2053 | 1.1529 | 1.1529 | 1 | 1 |
| WCM369 | 11 | 62285912 | 66099717 | −0.0814 | NA | −0.0525 | −0.0525 | 0 | 0 |
| WCM369 | 11 | 66100104 | 134605849 | 0.0378 | 0.1206 | 1.0872 | 1.0872 | 1 | 1 |
| WCM369 | 12 | 148444 | 133810886 | 0.0184 | −0.1594 | 0.8954 | 0.8954 | 1 | 1 |
| WCM369 | 13 | 19042044 | 115089411 | 0.0269 | −0.0298 | 0.9795 | 0.9795 | 1 | 1 |
| WCM369 | 14 | 19110231 | 107283269 | 0.0231 | −0.0869 | 0.9415 | 0.9415 | 1 | 1 |
| WCM369 | 15 | 20084061 | 102506264 | 0.0366 | 0.1046 | 1.0752 | 1.0752 | 1 | 1 |
| WCM369 | 16 | 64400 | 90260461 | 0.0351 | 0.0842 | 1.0601 | 1.0601 | 1 | 1 |
| WCM369 | 17 | 131595 | 81052609 | 0.0157 | −0.2027 | 0.8689 | 0.8689 | 1 | 1 |
| WCM369 | 18 | 47790 | 78005160 | 0.0199 | −0.1361 | 0.9099 | 0.9099 | 1 | 1 |
| WCM369 | 19 | 105010 | 59093696 | −0.0018 | −0.5189 | 0.6979 | 0.6979 | 1 | 1 |
| WCM369 | 2 | 41662 | 243037045 | 0.0126 | −0.2545 | 0.8383 | 0.8383 | 1 | 1 |
| WCM369 | 20 | 68446 | 62934541 | 0.0414 | 0.1682 | 1.1236 | 1.1236 | 1 | 1 |
| WCM369 | 21 | 9590305 | 48084202 | 0.0115 | −0.2719 | 0.8282 | 0.8282 | 1 | 1 |
| WCM369 | 22 | 16158982 | 51219129 | 0.0615 | 0.4062 | 1.3252 | 1.3252 | 1 | 1 |
| WCM369 | 3 | 239436 | 197896818 | 0.022 | −0.1028 | 0.9312 | 0.9312 | 1 | 1 |
| WCM369 | 4 | 53336 | 190878479 | 0.0215 | −0.1108 | 0.9261 | 0.9261 | 1 | 1 |
| WCM369 | 5 | 90680 | 180687818 | 0.0104 | −0.2912 | 0.8172 | 0.8172 | 1 | 1 |
| WCM369 | 6 | 203374 | 170892848 | 0.0136 | −0.2367 | 0.8487 | 0.8487 | 1 | 1 |
| WCM369 | 7 | 193245 | 158937161 | 0.0191 | −0.1488 | 0.902 | 0.902 | 1 | 1 |
| WCM369 | 8 | 163432 | 146279472 | 0.0115 | −0.2722 | 0.828 | 0.828 | 1 | 1 |
| WCM369 | 9 | 14859 | 141107519 | 0.0405 | 0.1567 | 1.1147 | 1.1147 | 1 | 1 |
| WCM369 | X | 2700202 | 154493585 | −0.0845 | NA | NA | NA | NA | NA |
| WCM369_Z2_1 | 1 | 721415 | 93029234 | 0.0048 | −0.0403 | 1.0157 | 0.9293 | 1 | 1 |
| WCM369_Z2_1 | 1 | 93070905 | 120468032 | −0.164 | −1.0132 | 0.9298 | 0.0612 | 1 | 0 |
| WCM369_Z2_1 | 1 | 120468280 | 249212402 | 0.0177 | 0.0159 | 1.046 | 0.9762 | 1 | 1 |
| WCM369_Z2_1 | 10 | 225979 | 108489811 | −0.0068 | −0.0928 | 0.9677 | 0.9077 | 1 | 1 |
| WCM369_Z2_1 | 10 | 108536376 | 124713689 | −0.1804 | −1.1459 | 0.9345 | −0.0307 | 1 | 0 |
| WCM369_Z2_1 | 10 | 124740140 | 135440068 | −0.0198 | −0.1531 | 0.9375 | 0.8611 | 1 | 1 |
| WCM369_Z2_1 | 11 | 193154 | 47862084 | 0.001 | −0.0575 | 1.0126 | 0.9092 | 1 | 1 |
| WCM369_Z2_1 | 11 | 47869483 | 63137597 | −0.1618 | −0.9962 | 1.0595 | −0.0568 | 1 | 0 |
| WCM369_Z2_1 | 11 | 63137897 | 134856597 | 0.005 | −0.0393 | 1.0077 | 0.9385 | 1 | 1 |
| WCM369_Z2_1 | 12 | 148637 | 46325313 | 0.018 | 0.0174 | 1.0906 | 0.9337 | 1 | 1 |
| WCM369_Z2_1 | 12 | 46339070 | 56120500 | 0.182 | 0.62 | 1.6541 | 1.4196 | 2 | 1 |
| WCM369_Z2_1 | 12 | 56120950 | 72893328 | 0.4489 | 1.339 | 2.6533 | 2.4064 | 3 | 2 |
| WCM369_Z2_1 | 12 | 72936128 | 110969446 | 0.1431 | 0.4926 | 3.0807 | −0.2667 | 3 | 0 |
| WCM369_Z2_1 | 12 | 110974890 | 133683077 | 0.3172 | 1.0121 | 2.9557 | 1.078 | 3 | 1 |
| WCM369_Z2_1 | 12 | 133696900 | 133810703 | 0.1838 | 0.6257 | NA | NA | NA | NA |
| WCM369_Z2_1 | 13 | 19600159 | 36767768 | −0.0327 | −0.2151 | 0.9625 | 0.7605 | 1 | 1 |
| WCM369_Z2_1 | 13 | 36776080 | 73637987 | −0.1838 | −1.175 | 0.8712 | 0.0145 | 1 | 0 |
| WCM369_Z2_1 | 13 | 73649988 | 115089411 | 0.0037 | −0.0454 | 1.0208 | 0.9172 | 1 | 1 |
| WCM369_Z2_1 | 14 | 19377710 | 107283034 | 0.0152 | 0.0053 | 1.0602 | 0.9471 | 1 | 1 |
| WCM369_Z2_1 | 15 | 20169971 | 102506273 | 0.0153 | 0.0055 | 1.3346 | 0.6731 | 1 | 1 |
| WCM369_Z2_1 | 16 | 67604 | 57953115 | 0.0331 | 0.0814 | 1.1198 | 0.9964 | 1 | 1 |
| WCM369_Z2_1 | 16 | 57954302 | 89620186 | 0.0315 | 0.0747 | 2.0609 | 0.0453 | 2 | 0 |
| WCM369_Z2_1 | 16 | 89620933 | 90162178 | 0.0378 | 0.1006 | 2.2302 | −0.0858 | 2 | 0 |
| WCM369_Z2_1 | 17 | 131595 | 1707988 | 0.0291 | 0.0646 | 1.0458 | 1.0458 | 1 | 1 |
| WCM369_Z2_1 | 17 | 1715316 | 17991379 | −0.1635 | −1.0093 | 1.0043 | −0.0107 | 1 | 0 |
| WCM369_Z2_1 | 17 | 17997248 | 81083419 | 0.027 | 0.0559 | 1.0878 | 0.9912 | 1 | 1 |
| WCM369_Z2_1 | 18 | 48206 | 18547017 | −0.0035 | −0.0776 | 1.0378 | 0.8574 | 1 | 1 |
| WCM369_Z2_1 | 18 | 18547759 | 44683862 | 0.1889 | 0.6419 | 1.7272 | 1.3935 | 2 | 1 |
| WCM369_Z2_1 | 18 | 44693042 | 78005215 | −0.1784 | −1.1293 | 1.0317 | −0.1174 | 1 | 0 |
| WCM369_Z2_1 | 19 | 281405 | 373513 | −0.2561 | −1.9559 | NA | NA | NA | NA |
| WCM369_Z2_1 | 19 | 374344 | 9268741 | −0.1678 | −1.0434 | 0.9835 | −0.0131 | 1 | 0 |
| WCM369_Z2_1 | 19 | 9269580 | 59083953 | 0.0117 | −0.0099 | 1.0236 | 0.9628 | 1 | 1 |
| WCM369_Z2_1 | 2 | 41623 | 249732 | −0.2823 | −2.3754 | NA | NA | NA | NA |
| WCM369_Z2_1 | 2 | 253112 | 219221890 | 0.009 | −0.0217 | 1.0284 | 0.9417 | 1 | 1 |
| WCM369_Z2_1 | 2 | 219222400 | 242946597 | −0.1501 | −0.9092 | 0.9826 | 0.0824 | 1 | 0 |
| WCM369_Z2_1 | 20 | 68397 | 62904848 | 0.0188 | 0.0206 | 1.0406 | 0.9882 | 1 | 1 |
| WCM369_Z2_1 | 21 | 9907318 | 48084210 | −0.0319 | −0.2114 | 0.9479 | 0.7795 | 1 | 1 |
| WCM369_Z2_1 | 22 | 17060362 | 20044100 | 0.0633 | 0.2032 | 1.2907 | 1.0118 | 1 | 1 |
| WCM369_Z2_1 | 22 | 20044324 | 29932632 | −0.1783 | −1.1286 | 0.9787 | −0.0639 | 1 | 0 |
| WCM369_Z2_1 | 22 | 29935409 | 43565703 | 0.0238 | 0.0421 | 1.1303 | 0.929 | 1 | 1 |
| WCM369_Z2_1 | 22 | 43565855 | 51215134 | −0.1695 | −1.0569 | 0.9133 | 0.048 | 1 | 0 |
| WCM369_Z2_1 | 3 | 239459 | 45715028 | 0.02 | 0.0257 | 1.0485 | 0.9874 | 1 | 1 |
| WCM369_Z2_1 | 3 | 45715850 | 97503819 | −0.1209 | −0.7087 | 0.6673 | 0.5564 | 1 | 1 |
| WCM369_Z2_1 | 3 | 97506924 | 132179158 | 0.0082 | −0.0253 | 1.0086 | 0.9566 | 1 | 1 |
| WCM369_Z2_1 | 3 | 132179936 | 197894726 | 0.1542 | 0.5299 | 1.5396 | 1.348 | 2 | 1 |
| WCM369_Z2_1 | 4 | 59434 | 190426502 | −0.0221 | −0.1638 | 0.9393 | 0.846 | 1 | 1 |
| WCM369_Z2_1 | 5 | 140657 | 52856504 | −0.0289 | −0.1967 | 0.9148 | 0.8303 | 1 | 1 |
| WCM369_Z2_1 | 5 | 52900714 | 74026175 | −0.2012 | −1.3305 | 0.7684 | 0.0268 | 1 | 0 |
| WCM369_Z2_1 | 5 | 74032597 | 180687812 | −0.0047 | −0.083 | 0.9956 | 0.8926 | 1 | 1 |
| WCM369_Z2_1 | 6 | 203510 | 170893407 | −0.029 | −0.1972 | 0.9271 | 0.8174 | 1 | 1 |
| WCM369_Z2_1 | 7 | 193222 | 158937375 | 0 | −0.0618 | 0.9925 | 0.9236 | 1 | 1 |
| WCM369_Z2_1 | 8 | 190907 | 17851057 | 0.0234 | 0.0405 | 1.2123 | 0.8447 | 1 | 1 |
| WCM369_Z2_1 | 8 | 17863777 | 28827860 | −0.1999 | −1.3183 | 0.8059 | −0.0039 | 1 | 0 |
| WCM369_Z2_1 | 8 | 28828030 | 146279508 | −3.00E−04 | −0.0634 | 1.0216 | 0.8924 | 1 | 1 |
| WCM369_Z2_1 | 9 | 17083 | 141111414 | −0.1918 | −1.2445 | 1.0478 | −0.2038 | 1 | 0 |
| WCM369_Z2_1 | X | 2707688 | 2835964 | 0.1385 | 0.477 | NA | NA | NA | NA |
| WCM369_Z2_1 | X | 2836181 | 57934478 | 0.2665 | 0.8731 | NA | NA | NA | NA |
| WCM369_Z2_1 | X | 57935422 | 68060181 | 1.7467 | 3.4728 | NA | NA | NA | NA |
| WCM369_Z2_1 | X | 68381640 | 154509348 | −0.0324 | −0.2137 | NA | NA | NA | NA |
| WCM4 | 1 | 14930 | 6160087 | 0.1301 | NA | NA | NA | NA | NA |
| WCM4 | 1 | 6161939 | 115400167 | −0.1131 | NA | NA | NA | NA | NA |
| WCM4 | 1 | 115401180 | 249213973 | 0.0886 | NA | NA | NA | NA | NA |
| WCM4 | 10 | 226116 | 42833224 | −0.0794 | NA | NA | NA | NA | NA |
| WCM4 | 10 | 42833380 | 88935651 | 0.0388 | NA | NA | NA | NA | NA |
| WCM4 | 10 | 88939943 | 135477904 | −0.096 | NA | NA | NA | NA | NA |
| WCM4 | 11 | 193062 | 47435995 | −0.0247 | NA | NA | NA | NA | NA |
| WCM4 | 11 | 47436351 | 75277858 | −0.1362 | NA | NA | NA | NA | NA |
| WCM4 | 11 | 75279846 | 134605954 | 0.0409 | NA | NA | NA | NA | NA |
| WCM4 | 12 | 148474 | 133810704 | 0.0289 | NA | NA | NA | NA | NA |
| WCM4 | 13 | 19041994 | 115089411 | −0.1285 | NA | NA | NA | NA | NA |
| WCM4 | 14 | 19110239 | 107283263 | 0.0085 | NA | NA | NA | NA | NA |
| WCM4 | 15 | 20083910 | 102514200 | −0.0356 | NA | NA | NA | NA | NA |
| WCM4 | 16 | 64400 | 28992738 | 0.0524 | NA | NA | NA | NA | NA |
| WCM4 | 16 | 28992924 | 90110967 | −0.1169 | NA | NA | NA | NA | NA |
| WCM4 | 16 | 90113379 | 90260461 | −0.0013 | NA | NA | NA | NA | NA |
| WCM4 | 17 | 131595 | 15449101 | −0.1304 | NA | NA | NA | NA | NA |
| WCM4 | 17 | 15457000 | 80918962 | −0.0071 | NA | NA | NA | NA | NA |
| WCM4 | 17 | 80923643 | 81187925 | −0.4072 | NA | NA | NA | NA | NA |
| WCM4 | 18 | 47725 | 78005199 | −0.1024 | NA | NA | NA | NA | NA |
| WCM4 | 19 | 105021 | 59093645 | −0.0188 | NA | NA | NA | NA | NA |
| WCM4 | 2 | 41686 | 98744672 | −0.1316 | NA | NA | NA | NA | NA |
| WCM4 | 2 | 98750402 | 212295835 | 0.0037 | NA | NA | NA | NA | NA |
| WCM4 | 2 | 212295875 | 243036990 | −0.1127 | NA | NA | NA | NA | NA |
| WCM4 | 20 | 68413 | 62934592 | 0.0731 | NA | NA | NA | NA | NA |
| WCM4 | 21 | 9590312 | 48084175 | 0.0154 | NA | NA | NA | NA | NA |
| WCM4 | 22 | 16159044 | 51219090 | −0.0573 | NA | NA | NA | NA | NA |
| WCM4 | 3 | 239495 | 134644799 | 0.0186 | NA | NA | NA | NA | NA |
| WCM4 | 3 | 134652841 | 197896714 | 0.2308 | NA | NA | NA | NA | NA |
| WCM4 | 4 | 53345 | 515522 | −0.1528 | NA | NA | NA | NA | NA |
| WCM4 | 4 | 515682 | 77941804 | −0.0128 | NA | NA | NA | NA | NA |
| WCM4 | 4 | 77949963 | 190878508 | −0.1558 | NA | NA | NA | NA | NA |
| WCM4 | 5 | 90750 | 180687818 | −0.0113 | NA | NA | NA | NA | NA |
| WCM4 | 6 | 203515 | 67069109 | −0.0134 | NA | NA | NA | NA | NA |
| WCM4 | 6 | 67859579 | 100957844 | −0.1758 | NA | NA | NA | NA | NA |
| WCM4 | 6 | 100960563 | 170892848 | −0.0126 | NA | NA | NA | NA | NA |
| WCM4 | 7 | 193205 | 158937135 | 0.0066 | NA | NA | NA | NA | NA |
| WCM4 | 8 | 163438 | 62366826 | −0.0635 | NA | NA | NA | NA | NA |
| WCM4 | 8 | 62371063 | 144621438 | 0.2659 | NA | NA | NA | NA | NA |
| WCM4 | 8 | 144623550 | 146279472 | 0.0303 | NA | NA | NA | NA | NA |
| WCM4 | 9 | 14878 | 20995821 | −0.1395 | NA | NA | NA | NA | NA |
| WCM4 | 9 | 21007152 | 141107513 | 0.0419 | NA | NA | NA | NA | NA |
| WCM4 | X | 2700194 | 154493598 | −0.0226 | NA | NA | NA | NA | NA |
| WCM4_X1_1 | 1 | 721476 | 5624154 | 0.1793 | 1.2493 | 2.5103 | 2.2442 | 3 | 2 |
| WCM4_X1_1 | 1 | 5716339 | 114634694 | −0.2199 | 0.0526 | 2.025 | 0.0492 | 2 | 0 |
| WCM4_X1_1 | 1 | 114636419 | 117509908 | 0.0705 | 0.9813 | 2.4369 | 1.5115 | 2 | 2 |
| WCM4_X1_1 | 1 | 117516944 | 145115820 | 0.327 | 1.5728 | 4.762 | 1.1876 | 5 | 1 |
| WCM4_X1_1 | 1 | 145209397 | 160012078 | 0.0918 | 1.0361 | 2.8855 | 1.2159 | 3 | 1 |
| WCM4_X1_1 | 1 | 160012313 | 201176977 | 0.1963 | 1.2886 | 3.9462 | 0.9395 | 4 | 1 |
| WCM4_X1_1 | 1 | 201177033 | 249212393 | 0.2893 | 1.4938 | 3.7257 | 1.9069 | 4 | 2 |
| WCM4_X1_1 | 10 | 225958 | 42907265 | −0.1018 | 0.4808 | 1.8973 | 0.8937 | 2 | 1 |
| WCM4_X1_1 | 10 | 42916247 | 88973968 | 0.0905 | 1.0328 | 2.1294 | 1.9626 | 2 | 2 |
| WCM4_X1_1 | 10 | 88975814 | 108924259 | −0.2391 | −0.027 | 1.9521 | 0.0108 | 2 | 0 |
| WCM4_X1_1 | 10 | 111625035 | 125805389 | −0.07 | 0.5824 | 1.6419 | 1.3528 | 2 | 1 |
| WCM4_X1_1 | 10 | 125805631 | 135440108 | −0.2105 | 0.0902 | 1.9826 | 0.1463 | 2 | 0 |
| WCM4_X1_1 | 11 | 193112 | 211203 | 0.011 | 0.8209 | NA | NA | NA | NA |
| WCM4_X1_1 | 11 | 212483 | 280464 | −0.1155 | 0.4357 | NA | NA | NA | NA |
| WCM4_X1_1 | 11 | 280767 | 48387559 | −0.0509 | 0.6412 | 2.1976 | 0.9216 | 2 | 1 |
| WCM4_X1_1 | 11 | 48387833 | 67200098 | −0.2074 | 0.1026 | 1.8794 | 0.268 | 2 | 0 |
| WCM4_X1_1 | 11 | 67200513 | 75298409 | −0.0739 | 0.57 | 2.9776 | −0.0085 | 3 | 0 |
| WCM4_X1_1 | 11 | 75298797 | 75851965 | −0.1463 | 0.3301 | NA | NA | NA | NA |
| WCM4_X1_1 | 11 | 75852095 | 134856592 | 0.1753 | 1.2399 | 2.478 | 2.2458 | 2 | 2 |
| WCM4_X1_1 | 12 | 148811 | 10870701 | 0.3412 | 1.6018 | 4.0586 | 2.012 | 4 | 2 |
| WCM4_X1_1 | 12 | 10871730 | 27470922 | 0.2032 | 1.3042 | 2.6048 | 2.3342 | 3 | 2 |
| WCM4_X1_1 | 12 | 27472282 | 50291309 | 0.0549 | 0.9404 | 2.031 | 1.8072 | 2 | 2 |
| WCM4_X1_1 | 12 | 50291799 | 133779971 | −0.0676 | 0.5898 | 1.9208 | 1.0892 | 2 | 1 |
| WCM4_X1_1 | 12 | 133780228 | 133810735 | 0.1609 | 1.2058 | NA | NA | NA | NA |
| WCM4_X1_1 | 13 | 19600515 | 31729729 | 0.0585 | 0.9499 | 1.9827 | 1.8807 | 2 | 2 |
| WCM4_X1_1 | 13 | 31732966 | 115089411 | −0.2362 | −0.0149 | 1.9623 | 0.0171 | 2 | 0 |
| WCM4_X1_1 | 14 | 19377711 | 55411144 | 0.0581 | 0.9489 | 1.9559 | 1.9049 | 2 | 2 |
| WCM4_X1_1 | 14 | 55422341 | 72932528 | −0.1035 | 0.4755 | 1.7321 | 1.0486 | 2 | 1 |
| WCM4_X1_1 | 14 | 72936731 | 107283039 | 0.0666 | 0.9712 | 2.0066 | 1.9143 | 2 | 2 |
| WCM4_X1_1 | 15 | 20169960 | 30337272 | −0.3626 | −0.6413 | 1.437 | −0.1547 | 1 | 0 |
| WCM4_X1_1 | 15 | 30384259 | 42162092 | 0.0457 | 0.9159 | 2.8469 | 0.9266 | 3 | 1 |
| WCM4_X1_1 | 15 | 42162683 | 53997323 | −0.0889 | 0.5226 | 1.5562 | 1.3169 | 2 | 1 |
| WCM4_X1_1 | 15 | 53998178 | 72050344 | −0.209 | 0.096 | 1.899 | 0.2387 | 2 | 0 |
| WCM4_X1_1 | 15 | 72057368 | 72454350 | −0.1687 | 0.2495 | NA | NA | NA | NA |
| WCM4_X1_1 | 15 | 72454688 | 79586159 | −0.0629 | 0.6045 | 1.6938 | 1.347 | 2 | 1 |
| WCM4_X1_1 | 15 | 79586361 | 102517678 | −0.2139 | 0.0766 | 1.0883 | 1.0208 | 1 | 1 |
| WCM4_X1_1 | 16 | 66926 | 48582018 | 0.0868 | 1.0234 | 2.0742 | 1.991 | 2 | 2 |
| WCM4_X1_1 | 16 | 48585330 | 90233642 | −0.2066 | 0.1057 | 2.0391 | 0.1129 | 2 | 0 |
| WCM4_X1_1 | 17 | 131595 | 4458164 | −0.0475 | 0.6514 | 2.954 | 0.1874 | 3 | 0 |
| WCM4_X1_1 | 17 | 4458515 | 15341453 | −0.2231 | 0.0396 | 2.0051 | 0.0505 | 2 | 0 |
| WCM4_X1_1 | 17 | 15343552 | 76554194 | −0.0548 | 0.6291 | 2.0839 | 1.0093 | 2 | 1 |
| WCM4_X1_1 | 17 | 76556956 | 81083561 | 0.0981 | 1.0523 | 2.1681 | 1.9795 | 2 | 2 |
| WCM4_X1_1 | 18 | 48245 | 254318 | −0.1582 | 0.2877 | NA | NA | NA | NA |
| WCM4_X1_1 | 18 | 259240 | 23866185 | −0.0327 | 0.6956 | 1.701 | 1.5382 | 2 | 2 |
| WCM4_X1_1 | 18 | 23866440 | 78005226 | −0.2385 | −0.0245 | 1.9791 | −0.0128 | 2 | 0 |
| WCM4_X1_1 | 19 | 281439 | 24309994 | −0.0509 | 0.641 | 2.1407 | 0.9781 | 2 | 1 |
| WCM4_X1_1 | 19 | 24310193 | 59083953 | 0.0624 | 0.9602 | 1.9762 | 1.9149 | 2 | 2 |
| WCM4_X1_1 | 2 | 41613 | 54587381 | −0.2267 | 0.0245 | 1.8916 | 0.1427 | 2 | 0 |
| WCM4_X1_1 | 2 | 54587596 | 68280249 | −0.0047 | 0.7768 | 2.543 | 0.8838 | 3 | 1 |
| WCM4_X1_1 | 2 | 68352475 | 96859079 | −0.1981 | 0.139 | 2.0144 | 0.1878 | 2 | 0 |
| WCM4_X1_1 | 2 | 96860719 | 99136591 | −0.019 | 0.7357 | NA | NA | NA | NA |
| WCM4_X1_1 | 2 | 99137194 | 116572392 | 0.1848 | 1.262 | 3.5724 | 1.224 | 4 | 1 |
| WCM4_X1_1 | 2 | 116573271 | 186610166 | 0.0373 | 0.8932 | 3.7965 | −0.082 | 4 | 0 |
| WCM4_X1_1 | 2 | 186611497 | 187713816 | −0.3782 | −0.7367 | NA | NA | NA | NA |
| WCM4_X1_1 | 2 | 188211116 | 189839262 | −0.2186 | 0.0578 | NA | NA | NA | NA |
| WCM4_X1_1 | 2 | 189849556 | 213790998 | −0.0849 | 0.5353 | 2.9078 | −0.0094 | 3 | 0 |
| WCM4_X1_1 | 2 | 213872233 | 232894702 | −0.2213 | 0.0468 | 1.9821 | 0.0839 | 2 | 0 |
| WCM4_X1_1 | 2 | 232952213 | 242946616 | −0.3799 | −0.7472 | 1.2053 | −0.0137 | 1 | 0 |
| WCM4_X1_1 | 20 | 68396 | 30115358 | 0.188 | 1.2693 | 3.8158 | 1.005 | 4 | 1 |
| WCM4_X1_1 | 20 | 30126122 | 62904848 | 0.0906 | 1.0332 | 3.9649 | 0.1281 | 4 | 0 |
| WCM4_X1_1 | 21 | 9826207 | 16340289 | 0.3148 | 1.5473 | 5.5291 | 0.3165 | 6 | 0 |
| WCM4_X1_1 | 21 | 16340513 | 41137780 | −0.1009 | 0.4839 | 1.855 | 0.942 | 2 | 1 |
| WCM4_X1_1 | 21 | 41142950 | 48084207 | 0.0719 | 0.985 | 2.144 | 1.8147 | 2 | 2 |
| WCM4_X1_1 | 22 | 16287439 | 17443751 | 0.0854 | 1.0199 | NA | NA | NA | NA |
| WCM4_X1_1 | 22 | 17444668 | 24086864 | 0.0745 | 0.9918 | 2.133 | 1.8443 | 2 | 2 |
| WCM4_X1_1 | 22 | 24087185 | 51215124 | −0.2025 | 0.1216 | 2.1133 | 0.0626 | 2 | 0 |
| WCM4_X1_1 | 3 | 239390 | 121303785 | −0.0633 | 0.6033 | 2.0065 | 1.0318 | 2 | 1 |
| WCM4_X1_1 | 3 | 121304902 | 123550262 | 0.1949 | 1.2852 | 3.7933 | 1.081 | 4 | 1 |
| WCM4_X1_1 | 3 | 123616390 | 134977951 | −0.0833 | 0.5406 | 1.5655 | 1.3437 | 2 | 1 |
| WCM4_X1_1 | 3 | 135720431 | 197894726 | 0.4794 | 1.8714 | 5.3857 | 1.932 | 5 | 2 |
| WCM4_X1_1 | 4 | 59376 | 338183 | −0.0823 | 0.5436 | NA | NA | NA | NA |
| WCM4_X1_1 | 4 | 343982 | 77676328 | 0.0753 | 0.9938 | 2.0224 | 1.9605 | 2 | 2 |
| WCM4_X1_1 | 4 | 77677694 | 190426459 | −0.2501 | −0.0742 | 1.9543 | −0.0546 | 2 | 0 |
| WCM4_X1_1 | 5 | 140608 | 9063206 | 0.4565 | 1.8284 | 4.304 | 2.7991 | 4 | 3 |
| WCM4_X1_1 | 5 | 9066648 | 45645434 | 0.1661 | 1.2182 | 2.7881 | 1.8652 | 3 | 2 |
| WCM4_X1_1 | 5 | 45695803 | 180687766 | −0.0831 | 0.5413 | 1.8897 | 1.0208 | 2 | 1 |
| WCM4_X1_1 | 6 | 203624 | 395927 | 0.0394 | 0.899 | NA | NA | NA | NA |
| WCM4_X1_1 | 6 | 397251 | 33140341 | −0.089 | 0.5224 | 1.8138 | 1.0589 | 2 | 1 |
| WCM4_X1_1 | 6 | 33141291 | 66205190 | 0.0677 | 0.9741 | 1.9645 | 1.9645 | 2 | 2 |
| WCM4_X1_1 | 6 | 68598968 | 107389972 | −0.2472 | −0.0617 | 1.8656 | 0.0507 | 2 | 0 |
| WCM4_X1_1 | 6 | 107390452 | 108370490 | −0.0208 | 0.7304 | NA | NA | NA | NA |
| WCM4_X1_1 | 6 | 108372400 | 158735083 | 0.0594 | 0.9523 | 1.9911 | 1.8788 | 2 | 2 |
| WCM4_X1_1 | 6 | 158834126 | 170893406 | −0.0701 | 0.582 | 2.0658 | 0.9279 | 2 | 1 |
| WCM4_X1_1 | 7 | 193245 | 11415429 | −0.0567 | 0.6233 | 1.9546 | 1.1262 | 2 | 1 |
| WCM4_X1_1 | 7 | 11416258 | 90675228 | 0.1008 | 1.0589 | 2.1359 | 2.0309 | 2 | 2 |
| WCM4_X1_1 | 7 | 90708790 | 95664996 | 0.3872 | 1.6943 | 4.7218 | 1.7508 | 5 | 2 |
| WCM4_X1_1 | 7 | 95668664 | 100674493 | 0.0857 | 1.0206 | 2.46 | 1.5975 | 2 | 2 |
| WCM4_X1_1 | 7 | 100674969 | 158937074 | −0.063 | 0.6042 | 2.0169 | 1.0233 | 2 | 1 |
| WCM4_X1_1 | 8 | 190897 | 17270785 | 0.0605 | 0.9551 | 2.0358 | 1.8418 | 2 | 2 |
| WCM4_X1_1 | 8 | 17396380 | 38028519 | −0.2298 | 0.0121 | 1.9812 | 0.0356 | 2 | 0 |
| WCM4_X1_1 | 8 | 38029513 | 61765560 | 0.0321 | 0.8792 | 1.9895 | 1.6892 | 2 | 2 |
| WCM4_X1_1 | 8 | 61765944 | 65527696 | 0.3593 | 1.6386 | NA | NA | NA | NA |
| WCM4_X1_1 | 8 | 65528414 | 141449204 | 0.5795 | 2.0528 | 6.3147 | 1.9837 | 6 | 2 |
| WCM4_X1_1 | 8 | 141461185 | 143999086 | 0.4292 | 1.7764 | 4.441 | 2.4105 | 4 | 2 |
| WCM4_X1_1 | 8 | 144063784 | 146279507 | 0.1039 | 1.0667 | 2.0947 | 2.0947 | 2 | 2 |
| WCM4_X1_1 | 9 | 17066 | 21409188 | −0.1418 | 0.3458 | 2.5134 | 0.0284 | 3 | 0 |
| WCM4_X1_1 | 9 | 21409656 | 141006951 | 0.0671 | 0.9726 | 2.0085 | 1.9162 | 2 | 2 |
| WCM4_X1_1 | 9 | 141008884 | 141111414 | −0.1567 | 0.2932 | NA | NA | NA | NA |
| WCM4_X1_1 | X | 2700125 | 6069337 | 0.4826 | 1.8773 | NA | NA | NA | NA |
| WCM4_X1_1 | X | 6301987 | 64137723 | 0.0422 | 0.9066 | NA | NA | NA | NA |
| WCM4_X1_1 | X | 64138973 | 67273525 | 0.711 | 2.2772 | NA | NA | NA | NA |
| WCM4_X1_1 | X | 67283750 | 154509348 | 0.0233 | 0.8552 | NA | NA | NA | NA |
| WCM6 | 1 | 17468 | 1635601 | 0.0695 | NA | NA | NA | NA | NA |
| WCM6 | 1 | 1644760 | 83451726 | −0.035 | NA | NA | NA | NA | NA |
| WCM6 | 1 | 83568637 | 86907151 | 0.0049 | NA | NA | NA | NA | NA |
| WCM6 | 1 | 86909567 | 145290346 | 0.0037 | NA | NA | NA | NA | NA |
| WCM6 | 1 | 145293515 | 249213973 | −0.0046 | NA | NA | NA | NA | NA |
| WCM6 | 10 | 226107 | 26355872 | −0.0325 | NA | NA | NA | NA | NA |
| WCM6 | 10 | 26357625 | 35320431 | −0.0181 | NA | NA | NA | NA | NA |
| WCM6 | 10 | 35321414 | 135438976 | −0.0199 | NA | NA | NA | NA | NA |
| WCM6 | 11 | 193096 | 134605610 | −0.0463 | NA | NA | NA | NA | NA |
| WCM6 | 12 | 148444 | 72288678 | −0.0531 | NA | NA | NA | NA | NA |
| WCM6 | 12 | 72289843 | 105568239 | −0.0589 | NA | NA | NA | NA | NA |
| WCM6 | 12 | 105569748 | 133810794 | −0.0687 | NA | NA | NA | NA | NA |
| WCM6 | 13 | 19041992 | 115070216 | −0.0442 | NA | NA | NA | NA | NA |
| WCM6 | 14 | 19110297 | 107283263 | −0.0482 | NA | NA | NA | NA | NA |
| WCM6 | 15 | 20083928 | 102506284 | −0.0279 | NA | NA | NA | NA | NA |
| WCM6 | 16 | 67688 | 90260461 | −0.0528 | NA | NA | NA | NA | NA |
| WCM6 | 17 | 131595 | 81187935 | −0.0473 | NA | NA | NA | NA | NA |
| WCM6 | 18 | 47823 | 78005135 | −0.05 | NA | NA | NA | NA | NA |
| WCM6 | 19 | 105021 | 59093533 | −0.0412 | NA | NA | NA | NA | NA |
| WCM6 | 2 | 41614 | 243037022 | −0.0358 | NA | NA | NA | NA | NA |
| WCM6 | 20 | 68413 | 62934572 | −0.0542 | NA | NA | NA | NA | NA |
| WCM6 | 21 | 9590301 | 48084211 | −0.0267 | NA | NA | NA | NA | NA |
| WCM6 | 22 | 16159021 | 51215098 | −0.0693 | NA | NA | NA | NA | NA |
| WCM6 | 3 | 361557 | 197894726 | −0.0154 | NA | NA | NA | NA | NA |
| WCM6 | 4 | 53379 | 190874234 | −0.0123 | NA | NA | NA | NA | NA |
| WCM6 | 5 | 90697 | 180687824 | −0.0351 | NA | NA | NA | NA | NA |
| WCM6 | 6 | 203397 | 13601066 | −0.0438 | NA | NA | NA | NA | NA |
| WCM6 | 6 | 13604710 | 34950511 | −0.0703 | NA | NA | NA | NA | NA |
| WCM6 | 6 | 34951233 | 170892883 | −0.0386 | NA | NA | NA | NA | NA |
| WCM6 | 7 | 193222 | 158937072 | −0.0422 | NA | NA | NA | NA | NA |
| WCM6 | 8 | 163474 | 146279349 | −0.002 | NA | NA | NA | NA | NA |
| WCM6 | 9 | 116841 | 141107533 | −0.0089 | NA | NA | NA | NA | NA |
| WCM6 | X | 2700194 | 154493484 | −0.1863 | NA | NA | NA | NA | NA |
| WCM63 | 1 | 14907 | 249213206 | 0.0131 | 0.1289 | 1.0935 | 1.0935 | 1 | 1 |
| WCM63 | 10 | 225988 | 135438960 | −0.0101 | −0.021 | 0.9856 | 0.9856 | 1 | 1 |
| WCM63 | 11 | 193096 | 134605906 | −0.0186 | −0.0794 | 0.9464 | 0.9464 | 1 | 1 |
| WCM63 | 12 | 148567 | 133810712 | −2.00E−04 | 0.0448 | 1.0315 | 1.0315 | 1 | 1 |
| WCM63 | 13 | 19041979 | 115089411 | −0.0753 | −0.5315 | 0.6919 | 0.6919 | 1 | 1 |
| WCM63 | 14 | 19110343 | 107283274 | −0.0036 | 0.0223 | 1.0156 | 1.0156 | 1 | 1 |
| WCM63 | 15 | 20083905 | 102513459 | −0.0343 | −0.1923 | 0.8752 | 0.8752 | 1 | 1 |
| WCM63 | 16 | 66881 | 310757 | −0.1359 | −1.2176 | 0.8263 | 0.0337 | 1 | 0 |
| WCM63 | 16 | 311469 | 50134196 | 0.0548 | 0.3695 | 1.2919 | 1.2919 | 1 | 1 |
| WCM63 | 16 | 50134334 | 90168697 | −0.0833 | −0.6065 | 0.6568 | 0.6568 | 1 | 1 |
| WCM63 | 17 | 131595 | 81187935 | 0.0021 | 0.0596 | 1.0422 | 1.0422 | 1 | 1 |
| WCM63 | 18 | 47654 | 78005150 | −0.0078 | −0.0054 | 0.9963 | 0.9963 | 1 | 1 |
| WCM63 | 19 | 105069 | 59093696 | −0.0521 | −0.3314 | 0.7948 | 0.7948 | 1 | 1 |
| WCM63 | 2 | 41602 | 243037003 | −0.0181 | −0.0759 | 0.9488 | 0.9488 | 1 | 1 |
| WCM63 | 20 | 68346 | 62934541 | 0.0145 | 0.1378 | 1.1003 | 1.1003 | 1 | 1 |
| WCM63 | 21 | 9590314 | 48084170 | −0.0025 | 0.0295 | 1.0206 | 1.0206 | 1 | 1 |
| WCM63 | 22 | 16159068 | 51219089 | 0.0075 | 0.0943 | 1.0675 | 1.0675 | 1 | 1 |
| WCM63 | 3 | 239524 | 197896800 | 0.055 | 0.3706 | 1.2928 | 1.2928 | 1 | 1 |
| WCM63 | 4 | 53364 | 52729266 | 0.0212 | 0.1781 | 1.1314 | 1.1314 | 1 | 1 |
| WCM63 | 4 | 52729812 | 82366788 | 0.2959 | 1.3546 | 2.6477 | 2.4668 | 3 | 2 |
| WCM63 | 4 | 82366976 | 190878479 | −0.0136 | −0.0447 | 0.9695 | 0.9695 | 1 | 1 |
| WCM63 | 5 | 90784 | 44310492 | 0.0703 | 0.4512 | 1.3672 | 1.3672 | 1 | 1 |
| WCM63 | 5 | 44388482 | 140866319 | −0.0876 | −0.6492 | 0.6376 | 0.6376 | 1 | 1 |
| WCM63 | 5 | 140866565 | 180687776 | 0.0175 | 0.1562 | 1.1144 | 1.1144 | 1 | 1 |
| WCM63 | 6 | 203475 | 170892918 | −0.0226 | −0.1076 | 0.9281 | 0.9281 | 1 | 1 |
| WCM63 | 7 | 193265 | 158937022 | 0.0452 | 0.317 | 1.2458 | 1.2458 | 1 | 1 |
| WCM63 | 8 | 163432 | 37607152 | −0.1036 | −0.8149 | 0.6121 | 0.5248 | 1 | 1 |
| WCM63 | 8 | 37607377 | 146003567 | 0.1243 | 0.7093 | 1.635 | 1.635 | 2 | 2 |
| WCM63 | 8 | 146005648 | 146279416 | −0.175 | −1.9032 | NA | NA | NA | NA |
| WCM63 | 9 | 116804 | 141107525 | 0.0128 | 0.1271 | 1.0921 | 1.0921 | 1 | 1 |
| WCM63 | X | 2700141 | 154493590 | −0.2258 | −4.0144 | NA | NA | NA | NA |
| WCM63_X1_1 | 1 | 69603 | 11169379 | 0.0999 | NA | NA | NA | NA | NA |
| WCM63_X1_1 | 1 | 11169776 | 43315345 | −0.0839 | NA | NA | NA | NA | NA |
| WCM63_X1_1 | 1 | 43316408 | 174606535 | 0.1084 | NA | NA | NA | NA | NA |
| WCM63_X1_1 | 1 | 174652731 | 249212550 | −0.0447 | NA | NA | NA | NA | NA |
| WCM63_X1_1 | 10 | 226021 | 135440086 | −0.0495 | NA | NA | NA | NA | NA |
| WCM63_X1_1 | 11 | 193146 | 134856523 | −0.0543 | NA | NA | NA | NA | NA |
| WCM63_X1_1 | 12 | 148644 | 133810703 | −0.0561 | NA | NA | NA | NA | NA |
| WCM63_X1_1 | 13 | 19600515 | 115089411 | 0.027 | NA | NA | NA | NA | NA |
| WCM63_X1_1 | 14 | 19377726 | 107283210 | 0.1078 | NA | NA | NA | NA | NA |
| WCM63_X1_1 | 15 | 20170150 | 102517660 | 0.0854 | NA | NA | NA | NA | NA |
| WCM63_X1_1 | 16 | 66953 | 90162166 | 0.0955 | NA | NA | NA | NA | NA |
| WCM63_X1_1 | 17 | 131595 | 81083507 | 0.1009 | NA | NA | NA | NA | NA |
| WCM63_X1_1 | 18 | 48236 | 78005215 | 0.0916 | NA | NA | NA | NA | NA |
| WCM63_X1_1 | 19 | 281535 | 59083953 | 0.0623 | NA | NA | NA | NA | NA |
| WCM63_X1_1 | 2 | 41612 | 242946607 | 0.0916 | NA | NA | NA | NA | NA |
| WCM63_X1_1 | 20 | 68363 | 62904932 | 0.0883 | NA | NA | NA | NA | NA |
| WCM63_X1_1 | 21 | 9907319 | 48084282 | 0.1182 | NA | NA | NA | NA | NA |
| WCM63_X1_1 | 22 | 16287491 | 51215107 | 0.074 | NA | NA | NA | NA | NA |
| WCM63_X1_1 | 3 | 239347 | 197894720 | 0.1278 | NA | NA | NA | NA | NA |
| WCM63_X1_1 | 4 | 59327 | 190426502 | −0.0175 | NA | NA | NA | NA | NA |
| WCM63_X1_1 | 5 | 140532 | 180687768 | −0.0564 | NA | NA | NA | NA | NA |
| WCM63_X1_1 | 6 | 203577 | 170893389 | −0.0536 | NA | NA | NA | NA | NA |
| WCM63_X1_1 | 7 | 193436 | 158937416 | −0.0506 | NA | NA | NA | NA | NA |
| WCM63_X1_1 | 8 | 190897 | 146279434 | −0.0324 | NA | NA | NA | NA | NA |
| WCM63_X1_1 | 9 | 116846 | 141111414 | −0.0704 | NA | NA | NA | NA | NA |
| WCM63_X1_1 | X | 2707709 | 154509348 | −0.1085 | NA | NA | NA | NA | NA |
| WCM63_Z1_1 | 1 | 69569 | 119444015 | −0.5313 | −0.0115 | 1.0108 | 0.9733 | 1 | 1 |
| WCM63_Z1_1 | 1 | 119466151 | 187610871 | 0.1372 | 0.7158 | 2.3052 | 0.9795 | 2 | 1 |
| WCM63_Z1_1 | 1 | 190067283 | 202532025 | −0.5489 | −0.031 | 1.0005 | 0.9569 | 1 | 1 |
| WCM63_Z1_1 | 1 | 202533582 | 214209028 | −0.0092 | 0.5589 | 1.9833 | 0.963 | 2 | 1 |
| WCM63_Z1_1 | 1 | 214454687 | 249212420 | −0.5702 | −0.0548 | 0.9778 | 0.9477 | 1 | 1 |
| WCM63_Z1_1 | 10 | 226016 | 135440081 | −0.5616 | −0.0452 | 0.9778 | 0.9605 | 1 | 1 |
| WCM63_Z1_1 | 11 | 193127 | 43419010 | −0.4779 | 0.0479 | 1.1207 | 0.9467 | 1 | 1 |
| WCM63_Z1_1 | 11 | 43419535 | 48286316 | −0.0803 | 0.4823 | 2.8115 | −0.0176 | 3 | 0 |
| WCM63_Z1_1 | 11 | 48327935 | 65625613 | 0.3382 | 0.9294 | 2.7672 | 1.0418 | 3 | 1 |
| WCM63_Z1_1 | 11 | 65625903 | 71139887 | 0.6443 | 1.2515 | 2.847 | 1.9149 | 3 | 2 |
| WCM63_Z1_1 | 11 | 71146195 | 107300116 | 0.3379 | 0.9291 | 2.7968 | 1.0114 | 3 | 1 |
| WCM63_Z1_1 | 11 | 107304401 | 126969232 | −0.1717 | 0.3834 | 2.618 | −0.0092 | 3 | 0 |
| WCM63_Z1_1 | 11 | 128332391 | 134856563 | 0.6312 | 1.2378 | 2.4522 | 2.2645 | 2 | 2 |
| WCM63_Z1_1 | 12 | 148642 | 45108506 | 0.555 | 1.1578 | 2.6224 | 1.8401 | 3 | 2 |
| WCM63_Z1_1 | 12 | 45168521 | 133810703 | −0.0332 | 0.5331 | 1.9285 | 0.9656 | 2 | 1 |
| WCM63_Z1_1 | 13 | 19600515 | 30854220 | −0.3051 | 0.2381 | 1.6879 | 0.6709 | 2 | 1 |
| WCM63_Z1_1 | 13 | 30857848 | 88329795 | −0.7767 | −0.2872 | 1.6524 | −0.0134 | 2 | 0 |
| WCM63_Z1_1 | 13 | 88329996 | 115089411 | −0.3603 | 0.1775 | 1.6488 | 0.613 | 2 | 1 |
| WCM63_Z1_1 | 14 | 19377710 | 39787206 | 0.0238 | 0.5944 | 2.0026 | 1.0171 | 2 | 1 |
| WCM63_Z1_1 | 14 | 39788417 | 64009521 | 0.3376 | 0.9287 | 1.9238 | 1.8835 | 2 | 2 |
| WCM63_Z1_1 | 14 | 64065707 | 94510964 | −0.4862 | 0.0386 | 2.0572 | −0.003 | 2 | 0 |
| WCM63_Z1_1 | 14 | 94512109 | 107283027 | 0.0433 | 0.6153 | 2.0494 | 1.0143 | 2 | 1 |
| WCM63_Z1_1 | 15 | 20170150 | 102517667 | −0.5243 | −0.0036 | 1.0696 | 0.9254 | 1 | 1 |
| WCM63_Z1_1 | 16 | 66926 | 50245357 | 0.5427 | 1.145 | 3.4563 | 0.9666 | 3 | 1 |
| WCM63_Z1_1 | 16 | 50250068 | 53738108 | −0.4434 | 0.086 | 1.2045 | 0.9184 | 1 | 1 |
| WCM63_Z1_1 | 16 | 53844066 | 67919945 | −1.3215 | −0.9245 | 1.0564 | −0.0027 | 1 | 0 |
| WCM63_Z1_1 | 16 | 67942698 | 68849468 | −0.4845 | 0.0405 | 2.0621 | −0.0051 | 2 | 0 |
| WCM63_Z1_1 | 16 | 68853266 | 74447514 | 0.0584 | 0.6315 | 1.9592 | 1.1391 | 2 | 1 |
| WCM63_Z1_1 | 16 | 74452059 | 90162163 | −0.7075 | −0.2088 | 1.7333 | −0.0027 | 2 | 0 |
| WCM63_Z1_1 | 17 | 131595 | 8090556 | −0.0227 | 0.5444 | 1.9528 | 0.964 | 2 | 1 |
| WCM63_Z1_1 | 17 | 8092612 | 81083455 | 0.3817 | 0.9754 | 1.9703 | 1.962 | 2 | 2 |
| WCM63_Z1_1 | 18 | 48209 | 23865917 | −0.1168 | 0.4429 | 1.8372 | 0.8814 | 2 | 1 |
| WCM63_Z1_1 | 18 | 23866346 | 78005224 | −0.5546 | −0.0374 | 1.2957 | 0.6531 | 1 | 1 |
| WCM63_Z1_1 | 19 | 281503 | 59083923 | 0.0139 | 0.5837 | 1.9865 | 1.0109 | 2 | 1 |
| WCM63_Z1_1 | 2 | 41612 | 11341155 | 0.1532 | 0.7328 | 1.7647 | 1.559 | 2 | 2 |
| WCM63_Z1_1 | 2 | 11341482 | 17692196 | −0.7778 | −0.2884 | 1.0439 | 0.5938 | 1 | 1 |
| WCM63_Z1_1 | 2 | 17696573 | 17888572 | 0.6026 | 1.2079 | NA | NA | NA | NA |
| WCM63_Z1_1 | 2 | 17889918 | 26863417 | 0.818 | 1.4328 | 3.8944 | 1.5051 | 4 | 2 |
| WCM63_Z1_1 | 2 | 26864126 | 47596234 | 0.5156 | 1.1165 | 2.9203 | 1.416 | 3 | 1 |
| WCM63_Z1_1 | 2 | 47596923 | 67630744 | −0.0999 | 0.4611 | 1.8541 | 0.899 | 2 | 1 |
| WCM63_Z1_1 | 2 | 67630982 | 74275496 | −0.5087 | 0.0136 | 1.0274 | 0.9916 | 1 | 1 |
| WCM63_Z1_1 | 2 | 74300717 | 74757554 | 0.0221 | 0.5925 | NA | NA | NA | NA |
| WCM63_Z1_1 | 2 | 74757804 | 80874991 | 0.5884 | 1.1929 | NA | NA | NA | NA |
| WCM63_Z1_1 | 2 | 84517973 | 96858129 | 0.2781 | 0.8657 | 1.8647 | 1.7797 | 2 | 2 |
| WCM63_Z1_1 | 2 | 96859034 | 116497429 | 0.6012 | 1.2063 | 2.7864 | 1.8286 | 3 | 2 |
| WCM63_Z1_1 | 2 | 116503691 | 125671779 | −0.5346 | −0.0151 | 1.9857 | −0.0065 | 2 | 0 |
| WCM63_Z1_1 | 2 | 127413867 | 141533723 | −1.4031 | −1.024 | 0.9903 | −0.0068 | 1 | 0 |
| WCM63_Z1_1 | 2 | 141571257 | 142237986 | 0.0405 | 0.6123 | NA | NA | NA | NA |
| WCM63_Z1_1 | 2 | 142567961 | 174131113 | 0.2498 | 0.8357 | 2.6397 | 0.9297 | 3 | 1 |
| WCM63_Z1_1 | 2 | 174131421 | 242946604 | −0.0226 | 0.5445 | 1.934 | 0.9829 | 2 | 1 |
| WCM63_Z1_1 | 20 | 68396 | 62904932 | 0.3719 | 0.9651 | 1.9521 | 1.9521 | 2 | 2 |
| WCM63_Z1_1 | 21 | 9907313 | 48084282 | −0.0663 | 0.4974 | 1.8624 | 0.9609 | 2 | 1 |
| WCM63_Z1_1 | 22 | 16287437 | 51215152 | 0.0045 | 0.5736 | 1.9776 | 0.9988 | 2 | 1 |
| WCM63_Z1_1 | 3 | 239377 | 15686250 | −0.5122 | 0.0098 | 1.0214 | 0.9923 | 1 | 1 |
| WCM63_Z1_1 | 3 | 15686532 | 21706501 | −0.0648 | 0.499 | 1.904 | 0.9224 | 2 | 1 |
| WCM63_Z1_1 | 3 | 21792384 | 33183909 | −0.4154 | 0.1169 | 1.1848 | 0.984 | 1 | 1 |
| WCM63_Z1_1 | 3 | 33194345 | 49298205 | 0.0172 | 0.5873 | 2.0059 | 0.999 | 2 | 1 |
| WCM63_Z1_1 | 3 | 49298375 | 56600990 | −0.4996 | 0.0237 | 1.0493 | 0.9839 | 1 | 1 |
| WCM63_Z1_1 | 3 | 56603355 | 62118332 | −0.0058 | 0.5626 | 1.9358 | 1.018 | 2 | 1 |
| WCM63_Z1_1 | 3 | 62142766 | 66428224 | 0.4881 | 1.0876 | NA | NA | NA | NA |
| WCM63_Z1_1 | 3 | 66430811 | 97888405 | 0.7136 | 1.3239 | 3.4003 | 1.6067 | 3 | 2 |
| WCM63_Z1_1 | 3 | 97888572 | 116163765 | 0.3188 | 0.9089 | 1.9194 | 1.8358 | 2 | 2 |
| WCM63_Z1_1 | 3 | 116569172 | 132036371 | −0.0748 | 0.4882 | 1.9492 | 0.8562 | 2 | 1 |
| WCM63_Z1_1 | 3 | 132051162 | 167183428 | 0.5384 | 1.1405 | 3.4968 | 0.9124 | 3 | 1 |
| WCM63_Z1_1 | 3 | 167184936 | 197894726 | 0.8185 | 1.4333 | 4.4833 | 0.918 | 4 | 1 |
| WCM63_Z1_1 | 4 | 59327 | 49063852 | −0.5442 | −0.0259 | 1.0599 | 0.9046 | 1 | 1 |
| WCM63_Z1_1 | 4 | 52709800 | 69056991 | 0.6821 | 1.291 | 3.8053 | 1.0886 | 4 | 1 |
| WCM63_Z1_1 | 4 | 69094507 | 82355821 | 0.9277 | 1.5469 | 3.8469 | 1.9968 | 4 | 2 |
| WCM63_Z1_1 | 4 | 82362035 | 83277787 | 0.6239 | 1.2302 | NA | NA | NA | NA |
| WCM63_Z1_1 | 4 | 83278007 | 130023828 | 0.0405 | 0.6123 | 1.7255 | 1.3319 | 2 | 1 |
| WCM63_Z1_1 | 4 | 130030652 | 135121668 | −2.4686 | −2.4987 | NA | NA | NA | NA |
| WCM63_Z1_1 | 4 | 135122024 | 143007392 | −0.9161 | −0.4465 | 1.4858 | −0.0182 | 1 | 0 |
| WCM63_Z1_1 | 4 | 143029296 | 189026071 | −0.3476 | 0.1914 | 1.3812 | 0.9025 | 1 | 1 |
| WCM63_Z1_1 | 4 | 189026391 | 190426502 | −0.056 | 0.5085 | NA | NA | NA | NA |
| WCM63_Z1_1 | 5 | 140532 | 45695884 | −0.4434 | 0.086 | 1.2385 | 0.8844 | 1 | 1 |
| WCM63_Z1_1 | 5 | 49698142 | 66480250 | −1.4152 | −1.0388 | 0.9674 | 0.0061 | 1 | 0 |
| WCM63_Z1_1 | 5 | 66481790 | 82406916 | −0.5682 | −0.0525 | 0.9943 | 0.9341 | 1 | 1 |
| WCM63_Z1_1 | 5 | 82491672 | 110730437 | −1.335 | −0.9409 | 1.0669 | −0.0252 | 1 | 0 |
| WCM63_Z1_1 | 5 | 110784859 | 112102072 | −3.2003 | −4.0035 | NA | NA | NA | NA |
| WCM63_Z1_1 | 5 | 112102961 | 115230851 | −1.3602 | −0.9716 | 1.0055 | 0.0143 | 1 | 0 |
| WCM63_Z1_1 | 5 | 115238635 | 115249078 | 0.2105 | 0.7939 | NA | NA | NA | NA |
| WCM63_Z1_1 | 5 | 115297939 | 130721295 | −1.3196 | −0.9222 | 1.0368 | 0.0186 | 1 | 0 |
| WCM63_Z1_1 | 5 | 130726741 | 180687776 | −0.5071 | 0.0155 | 1.0209 | 1.0007 | 1 | 1 |
| WCM63_Z1_1 | 6 | 203479 | 393152 | 0.4715 | 1.0702 | NA | NA | NA | NA |
| WCM63_Z1_1 | 6 | 394847 | 18572091 | 0.3236 | 0.9139 | 1.9261 | 1.8423 | 2 | 2 |
| WCM63_Z1_1 | 6 | 19838312 | 84573910 | −0.0491 | 0.5159 | 1.8891 | 0.9707 | 2 | 1 |
| WCM63_Z1_1 | 6 | 84618797 | 88346163 | −0.6576 | −0.1526 | NA | NA | NA | NA |
| WCM63_Z1_1 | 6 | 88360928 | 170893408 | −0.099 | 0.4621 | 1.8226 | 0.9324 | 2 | 1 |
| WCM63_Z1_1 | 7 | 193245 | 57529304 | 0.5785 | 1.1826 | 2.6603 | 1.8793 | 3 | 2 |
| WCM63_Z1_1 | 7 | 57529628 | 158937400 | −0.0796 | 0.483 | 1.8458 | 0.9496 | 2 | 1 |
| WCM63_Z1_1 | 8 | 190907 | 37593994 | 0.228 | 0.8125 | 3.5117 | 9.00E−04 | 4 | 0 |
| WCM63_Z1_1 | 8 | 37595535 | 37632892 | 1.3761 | 2.0098 | NA | NA | NA | NA |
| WCM63_Z1_1 | 8 | 37633533 | 43197370 | 0.8718 | 1.4888 | 3.2646 | 2.3485 | 3 | 2 |
| WCM63_Z1_1 | 8 | 47752739 | 52325767 | 1.5551 | 2.1935 | NA | NA | NA | NA |
| WCM63_Z1_1 | 8 | 52336239 | 59720311 | 0.7191 | 1.3297 | 4.9805 | 0.0464 | 5 | 0 |
| WCM63_Z1_1 | 8 | 59720741 | 92212918 | 1.1924 | 1.8207 | 5.2342 | 1.831 | 5 | 2 |
| WCM63_Z1_1 | 8 | 92219027 | 98289897 | 0.9191 | 1.5379 | 4.8965 | 0.9109 | 5 | 1 |
| WCM63_Z1_1 | 8 | 98656803 | 110255528 | 1.5198 | 2.1573 | 4.5521 | 4.3692 | 5 | 4 |
| WCM63_Z1_1 | 8 | 110283353 | 116426487 | 1.1007 | 1.726 | 5.1409 | 1.4754 | 5 | 1 |
| WCM63_Z1_1 | 8 | 116426675 | 146279527 | 0.7817 | 1.395 | 5.2614 | −0.0015 | 5 | 0 |
| WCM63_Z1_1 | 9 | 116846 | 84528514 | −0.2083 | 0.3436 | 1.5615 | 0.9764 | 2 | 1 |
| WCM63_Z1_1 | 9 | 84529352 | 141111414 | −0.0611 | 0.503 | 1.8894 | 0.9449 | 2 | 1 |
| WCM63_Z1_1 | X | 2707715 | 22292191 | −0.6009 | −0.0891 | NA | NA | NA | NA |
| WCM63_Z1_1 | X | 22292259 | 27765165 | 0.3187 | 0.9088 | NA | NA | NA | NA |
| WCM63_Z1_1 | X | 27765759 | 48801499 | −0.645 | −0.1384 | NA | NA | NA | NA |
| WCM63_Z1_1 | X | 48814257 | 50378620 | 0.4123 | 1.0077 | NA | NA | NA | NA |
| WCM63_Z1_1 | X | 50556923 | 154509348 | −0.5951 | −0.0826 | NA | NA | NA | NA |
| WCM63_Z19_1 | 1 | 69590 | 249212401 | 6.00E−04 | 0.058 | 1.4412 | 0.6409 | 1 | 1 |
| WCM63_Z19_1 | 10 | 226048 | 135440080 | −0.0276 | NA | −0.4023 | −1.3834 | 0 | −1 |
| WCM63_Z19_1 | 11 | 193106 | 134856603 | −0.0251 | NA | −0.0366 | −1.4124 | 0 | −1 |
| WCM63_Z19_1 | 12 | 148674 | 133810703 | −0.0278 | NA | −0.2203 | −1.5936 | 0 | −2 |
| WCM63_Z19_1 | 13 | 19600515 | 115089411 | −0.025 | NA | 0.5628 | −1.9926 | 1 | −2 |
| WCM63_Z19_1 | 14 | 19377701 | 107283210 | −5.00E−04 | −0.0482 | 2.0668 | −0.1325 | 2 | 0 |
| WCM63_Z19_1 | 15 | 20170150 | 102517639 | 0.0019 | 0.1791 | 1.8331 | 0.4312 | 2 | 0 |
| WCM63_Z19_1 | 16 | 66966 | 90162169 | 0.0358 | 1.8113 | 3.5095 | 3.5095 | 4 | 4 |
| WCM63_Z19_1 | 17 | 131595 | 81083455 | 0.0395 | 1.916 | 4.4933 | 3.0545 | 4 | 3 |
| WCM63_Z19_1 | 18 | 48236 | 78005210 | −0.0155 | NA | 0.523 | −0.6641 | 1 | −1 |
| WCM63_Z19_1 | 19 | 281403 | 59083968 | 0.0482 | 2.1368 | 5.0182 | 3.7774 | 5 | 4 |
| WCM63_Z19_1 | 2 | 41615 | 242946589 | −5.00E−04 | −0.046 | 1.1685 | 0.7686 | 1 | 1 |
| WCM63_Z19_1 | 20 | 68351 | 62904925 | 0.0292 | 1.6078 | 3.0479 | 3.0479 | 3 | 3 |
| WCM63_Z19_1 | 21 | 9907283 | 48084277 | −0.011 | −2.0575 | 1.1334 | −0.6529 | 1 | −1 |
| WCM63_Z19_1 | 22 | 16287434 | 51215152 | 0.0247 | 1.4459 | 3.3347 | 2.114 | 3 | 2 |
| WCM63_Z19_1 | 3 | 239436 | 197894719 | 0.0123 | 0.8926 | 2.3608 | 1.3522 | 2 | 1 |
| WCM63_Z19_1 | 4 | 59345 | 190426502 | −0.0301 | NA | −0.2828 | −1.8498 | 0 | −2 |
| WCM63_Z19_1 | 5 | 140532 | 180687800 | −0.0246 | NA | −0.1992 | −1.1823 | 0 | −1 |
| WCM63_Z19_1 | 6 | 203509 | 170893411 | −0.0382 | NA | −0.836 | −2.3941 | −1 | −2 |
| WCM63_Z19_1 | 7 | 193451 | 158937416 | −0.0298 | NA | −0.3609 | −1.7322 | 0 | −2 |
| WCM63_Z19_1 | 8 | 190907 | 146279481 | −0.02 | NA | 0.2134 | −0.97 | 0 | −1 |
| WCM63_Z19_1 | 9 | 116938 | 141111414 | −0.0328 | NA | −0.76 | −1.7375 | −1 | −2 |
| WCM63_Z19_1 | X | 2707716 | 154509348 | −0.0797 | NA | NA | NA | NA | NA |
| WCM63_Z19_2 | 1 | 69649 | 84640739 | −0.1806 | 0.6087 | 2.082 | 0.9678 | 2 | 1 |
| WCM63_Z19_2 | 1 | 84641480 | 104114376 | −0.6114 | −0.0114 | 2.0881 | −0.1038 | 2 | 0 |
| WCM63_Z19_2 | 1 | 104114732 | 111217270 | −0.1438 | 0.6583 | 2.1835 | 0.9728 | 2 | 1 |
| WCM63_Z19_2 | 1 | 111434093 | 117307165 | −0.4918 | 0.1694 | 2.1207 | 0.1284 | 2 | 0 |
| WCM63_Z19_2 | 1 | 117311331 | 119427530 | −0.2246 | 0.5488 | 1.9212 | 1.0046 | 2 | 1 |
| WCM63_Z19_2 | 1 | 119427687 | 120387120 | 0.1875 | 1.0871 | NA | NA | NA | NA |
| WCM63_Z19_2 | 1 | 120436981 | 186329455 | 0.411 | 1.3621 | 3.1459 | 1.9955 | 3 | 2 |
| WCM63_Z19_2 | 1 | 186329951 | 202406993 | 0.1041 | 0.9818 | 2.0428 | 1.9071 | 2 | 2 |
| WCM63_Z19_2 | 1 | 202407199 | 205211164 | 0.4284 | 1.3833 | 3.1278 | 2.0893 | 3 | 2 |
| WCM63_Z19_2 | 1 | 205215773 | 214209123 | 0.1215 | 1.0039 | 2.0309 | 1.98 | 2 | 2 |
| WCM63_Z19_2 | 1 | 214454711 | 249212402 | −0.2259 | 0.547 | 1.9509 | 0.9712 | 2 | 1 |
| WCM63_Z19_2 | 10 | 226021 | 135440080 | −0.21 | 0.5687 | 2.0351 | 0.9313 | 2 | 1 |
| WCM63_Z19_2 | 11 | 193112 | 36614397 | −0.2175 | 0.5586 | 2.0279 | 0.9177 | 2 | 1 |
| WCM63_Z19_2 | 11 | 36614835 | 48286303 | −0.5658 | 0.0585 | 2.1505 | −0.0677 | 2 | 0 |
| WCM63_Z19_2 | 11 | 48327975 | 59834432 | −0.1571 | 0.6403 | 2.0284 | 1.0889 | 2 | 1 |
| WCM63_Z19_2 | 11 | 59837097 | 65547781 | 0.1284 | 1.0127 | 2.0407 | 1.9947 | 2 | 2 |
| WCM63_Z19_2 | 11 | 65547974 | 71155945 | 0.6307 | 1.6239 | 4.1575 | 2.0068 | 4 | 2 |
| WCM63_Z19_2 | 11 | 71164394 | 93463117 | 0.1448 | 1.0335 | 2.0832 | 2.0108 | 2 | 2 |
| WCM63_Z19_2 | 11 | 93463415 | 107299831 | −0.1836 | 0.6046 | 1.9283 | 1.1129 | 2 | 1 |
| WCM63_Z19_2 | 11 | 107300172 | 126969289 | −0.6102 | −0.0095 | 2.0673 | −0.0804 | 2 | 0 |
| WCM63_Z19_2 | 11 | 128332352 | 134856604 | 0.1521 | 1.0427 | 2.1172 | 2.0029 | 2 | 2 |
| WCM63_Z19_2 | 12 | 148675 | 43793017 | 0.1014 | 0.9785 | 1.9929 | 1.9478 | 2 | 2 |
| WCM63_Z19_2 | 12 | 43817588 | 50867846 | −0.2038 | 0.5772 | 2.0422 | 0.9417 | 2 | 1 |
| WCM63_Z19_2 | 12 | 50869561 | 67197936 | 0.0282 | 0.8845 | 1.8771 | 1.8151 | 2 | 2 |
| WCM63_Z19_2 | 12 | 67663563 | 106848421 | −0.235 | 0.5346 | 1.8896 | 1.0074 | 2 | 1 |
| WCM63_Z19_2 | 12 | 106851001 | 108642092 | −0.1285 | 0.6787 | NA | NA | NA | NA |
| WCM63_Z19_2 | 12 | 108675125 | 111760276 | 0.3994 | 1.3482 | 3.2337 | 1.8581 | 3 | 2 |
| WCM63_Z19_2 | 12 | 111772250 | 116406796 | −0.2247 | 0.5487 | 1.943 | 0.9825 | 2 | 1 |
| WCM63_Z19_2 | 12 | 116408538 | 121471337 | 0.3657 | 1.3072 | 2.6072 | 2.3421 | 3 | 2 |
| WCM63_Z19_2 | 12 | 121471525 | 122763598 | 0.0464 | 0.908 | 1.9586 | 1.7944 | 2 | 2 |
| WCM63_Z19_2 | 12 | 122773055 | 123968766 | −0.1835 | 0.6047 | 2.1076 | 0.9338 | 2 | 1 |
| WCM63_Z19_2 | 12 | 123969764 | 124821696 | 0.2169 | 1.124 | 2.2882 | 2.0707 | 2 | 2 |
| WCM63_Z19_2 | 12 | 124824663 | 132391447 | −0.1955 | 0.5884 | 2.0815 | 0.9257 | 2 | 1 |
| WCM63_Z19_2 | 12 | 132392049 | 133810703 | 0.1388 | 1.0259 | 2.0902 | 1.9821 | 2 | 2 |
| WCM63_Z19_2 | 13 | 19600515 | 30854269 | −0.2197 | 0.5555 | 1.8974 | 1.042 | 2 | 1 |
| WCM63_Z19_2 | 13 | 30857900 | 88329759 | −0.6282 | −0.0374 | 2.0274 | −0.0785 | 2 | 0 |
| WCM63_Z19_2 | 13 | 88330022 | 115089411 | −0.1542 | 0.6443 | 2.1304 | 0.9956 | 2 | 1 |
| WCM63_Z19_2 | 14 | 19377718 | 34993982 | 0.0998 | 0.9763 | 2.0226 | 1.9123 | 2 | 2 |
| WCM63_Z19_2 | 14 | 34998655 | 107283080 | −0.2069 | 0.573 | 2.0063 | 0.9689 | 2 | 1 |
| WCM63_Z19_2 | 15 | 20170150 | 22382897 | −0.6885 | −0.132 | 1.4524 | 0.3728 | 1 | 0 |
| WCM63_Z19_2 | 15 | 22383189 | 34380331 | −0.1777 | 0.6126 | 2.079 | 0.9791 | 2 | 1 |
| WCM63_Z19_2 | 15 | 34382621 | 102517646 | −0.5589 | 0.0689 | 1.1109 | 0.987 | 1 | 1 |
| WCM63_Z19_2 | 16 | 66955 | 27353498 | 0.4571 | 1.4178 | 4.2544 | 1.0891 | 4 | 1 |
| WCM63_Z19_2 | 16 | 27356242 | 50250077 | 0.679 | 1.6805 | 4.4559 | 1.955 | 4 | 2 |
| WCM63_Z19_2 | 16 | 50251538 | 53738097 | 0.16 | 1.0526 | 2.1396 | 2.009 | 2 | 2 |
| WCM63_Z19_2 | 16 | 53844114 | 69602441 | −0.5505 | 0.0816 | 2.1614 | −0.0451 | 2 | 0 |
| WCM63_Z19_2 | 16 | 69660405 | 73177675 | 0.2376 | 1.1497 | 2.2352 | 2.2021 | 2 | 2 |
| WCM63_Z19_2 | 16 | 73517324 | 90162177 | −0.5355 | 0.1043 | 2.1543 | −0.0044 | 2 | 0 |
| WCM63_Z19_2 | 17 | 131595 | 25636295 | −0.1238 | 0.685 | 2.1271 | 1.0883 | 2 | 1 |
| WCM63_Z19_2 | 17 | 25639382 | 81083419 | 0.1933 | 1.0944 | 2.1352 | 2.1352 | 2 | 2 |
| WCM63_Z19_2 | 18 | 48209 | 225134 | −0.0837 | 0.7383 | NA | NA | NA | NA |
| WCM63_Z19_2 | 18 | 225400 | 12785819 | 0.1943 | 1.0956 | 2.236 | 2.0381 | 2 | 2 |
| WCM63_Z19_2 | 18 | 12788127 | 78005210 | −0.1593 | 0.6374 | 2.0665 | 1.0446 | 2 | 1 |
| WCM63_Z19_2 | 19 | 281491 | 59083951 | −0.1253 | 0.683 | 2.1522 | 1.0588 | 2 | 1 |
| WCM63_Z19_2 | 2 | 41615 | 11315092 | 0.1456 | 1.0344 | 3.1503 | 0.9464 | 3 | 1 |
| WCM63_Z19_2 | 2 | 11317937 | 16745309 | −1.0517 | −0.7622 | 1.1085 | 0.0707 | 1 | 0 |
| WCM63_Z19_2 | 2 | 16746986 | 17888615 | −0.1346 | 0.6706 | NA | NA | NA | NA |
| WCM63_Z19_2 | 2 | 17889918 | 44104808 | 0.1393 | 1.0264 | 2.073 | 2.0009 | 2 | 2 |
| WCM63_Z19_2 | 2 | 44105051 | 68365887 | −0.2757 | 0.4785 | 1.9033 | 0.8833 | 2 | 1 |
| WCM63_Z19_2 | 2 | 68368831 | 72707874 | −0.5652 | 0.0594 | 2.1104 | −0.0264 | 2 | 0 |
| WCM63_Z19_2 | 2 | 72719504 | 74328649 | −1.1239 | −0.9035 | 1.0464 | 0.0227 | 1 | 0 |
| WCM63_Z19_2 | 2 | 74328895 | 74777384 | −0.5224 | 0.1238 | NA | NA | NA | NA |
| WCM63_Z19_2 | 2 | 74779681 | 84954858 | −0.1757 | 0.6153 | NA | NA | NA | NA |
| WCM63_Z19_2 | 2 | 84960570 | 116497383 | 0.1157 | 0.9966 | 2.2741 | 1.7166 | 2 | 2 |
| WCM63_Z19_2 | 2 | 116503691 | 125669078 | −0.5285 | 0.1147 | 2.1236 | 0.0419 | 2 | 0 |
| WCM63_Z19_2 | 2 | 125671671 | 141625760 | −1.1424 | −0.9411 | 1.0717 | −0.03 | 1 | 0 |
| WCM63_Z19_2 | 2 | 141641548 | 242946607 | −0.1733 | 0.6186 | 2.1015 | 0.9692 | 2 | 1 |
| WCM63_Z19_2 | 20 | 68396 | 29632635 | −0.5587 | 0.0693 | 1.0682 | 1.0301 | 1 | 1 |
| WCM63_Z19_2 | 20 | 29845473 | 62904925 | 0.2224 | 1.1308 | 2.2035 | 2.1762 | 2 | 2 |
| WCM63_Z19_2 | 21 | 9907291 | 48084277 | 0.1621 | 1.0553 | 2.1121 | 2.0441 | 2 | 2 |
| WCM63_Z19_2 | 22 | 16287488 | 51215120 | −0.1523 | 0.6469 | 2.1024 | 1.0292 | 2 | 1 |
| WCM63_Z19_2 | 3 | 239459 | 15686776 | −0.1242 | 0.6844 | 2.0855 | 1.1286 | 2 | 1 |
| WCM63_Z19_2 | 3 | 15686992 | 33617733 | 0.1478 | 1.0372 | 2.6403 | 1.4642 | 3 | 1 |
| WCM63_Z19_2 | 3 | 33618724 | 42632410 | 0.4508 | 1.4103 | 3.1725 | 2.1432 | 3 | 2 |
| WCM63_Z19_2 | 3 | 42633050 | 49294426 | 0.1807 | 1.0786 | 3.2014 | 1.0227 | 3 | 1 |
| WCM63_Z19_2 | 3 | 49294675 | 56653467 | −0.527 | 0.1169 | 1.1299 | 1.039 | 1 | 1 |
| WCM63_Z19_2 | 3 | 56653845 | 73432623 | 0.1017 | 0.9787 | 2.9242 | 1.0172 | 3 | 1 |
| WCM63_Z19_2 | 3 | 73432984 | 121217381 | 0.4254 | 1.3796 | 3.1547 | 2.0492 | 3 | 2 |
| WCM63_Z19_2 | 3 | 121229015 | 121339424 | 0.1169 | 0.9981 | NA | NA | NA | NA |
| WCM63_Z19_2 | 3 | 121340285 | 129152975 | 0.6982 | 1.703 | 3.2899 | 3.2216 | 3 | 3 |
| WCM63_Z19_2 | 3 | 129155463 | 197894713 | 0.8149 | 1.8381 | 4.0155 | 3.1351 | 4 | 3 |
| WCM63_Z19_2 | 4 | 59434 | 49019314 | 0.1582 | 1.0503 | 2.1257 | 2.0161 | 2 | 2 |
| WCM63_Z19_2 | 4 | 49030719 | 82377890 | 1.1132 | 2.1763 | 7.8334 | 1.2067 | 8 | 1 |
| WCM63_Z19_2 | 4 | 82378764 | 133368267 | −0.2298 | 0.5417 | 1.9841 | 0.9272 | 2 | 1 |
| WCM63_Z19_2 | 4 | 134071300 | 143045743 | −1.1842 | −1.0273 | NA | NA | NA | NA |
| WCM63_Z19_2 | 4 | 143067127 | 153245499 | 0.5018 | 1.4713 | 4.829 | 0.7165 | 5 | 1 |
| WCM63_Z19_2 | 4 | 153247278 | 178911632 | −0.134 | 0.6714 | 2.0934 | 1.0918 | 2 | 1 |
| WCM63_Z19_2 | 4 | 183090524 | 189060715 | −0.6223 | −0.0282 | 1.0323 | 0.929 | 1 | 1 |
| WCM63_Z19_2 | 4 | 189061024 | 190426502 | −0.4171 | 0.2785 | NA | NA | NA | NA |
| WCM63_Z19_2 | 5 | 140532 | 45695818 | 0.167 | 1.0614 | 2.3495 | 1.8244 | 2 | 2 |
| WCM63_Z19_2 | 5 | 49698128 | 66492459 | −0.651 | −0.0728 | 1.892 | 0.0096 | 2 | 0 |
| WCM63_Z19_2 | 5 | 67494070 | 82360047 | 0.0381 | 0.8973 | 1.8985 | 1.8265 | 2 | 2 |
| WCM63_Z19_2 | 5 | 82360839 | 130762977 | −0.615 | −0.0168 | 2.0419 | −0.0651 | 2 | 0 |
| WCM63_Z19_2 | 5 | 130764604 | 180687803 | 0.1132 | 0.9934 | 2.0061 | 1.9757 | 2 | 2 |
| WCM63_Z19_2 | 6 | 203535 | 393308 | 0.5438 | 1.5213 | NA | NA | NA | NA |
| WCM63_Z19_2 | 6 | 394889 | 19838425 | 0.2066 | 1.1111 | 2.4839 | 1.8363 | 2 | 2 |
| WCM63_Z19_2 | 6 | 19838843 | 84618750 | −0.1938 | 0.5907 | 2.0274 | 0.9847 | 2 | 1 |
| WCM63_Z19_2 | 6 | 84624165 | 88346138 | −0.7325 | −0.2027 | NA | NA | NA | NA |
| WCM63_Z19_2 | 6 | 88360921 | 170893411 | −0.2115 | 0.5667 | 1.972 | 0.9904 | 2 | 1 |
| WCM63_Z19_2 | 7 | 193245 | 57510008 | 0.3264 | 1.2593 | 3.8657 | 0.9218 | 4 | 1 |
| WCM63_Z19_2 | 7 | 57522211 | 158937400 | 0.0373 | 0.8963 | 2.0437 | 1.6789 | 2 | 2 |
| WCM63_Z19_2 | 8 | 190908 | 37456103 | −0.643 | −0.0604 | 1.9743 | −0.0563 | 2 | 0 |
| WCM63_Z19_2 | 8 | 37553685 | 59720303 | 0.5358 | 1.5118 | 4.2585 | 1.4447 | 4 | 1 |
| WCM63_Z19_2 | 8 | 59720709 | 103301755 | 0.6887 | 1.6919 | 3.8338 | 2.6277 | 4 | 3 |
| WCM63_Z19_2 | 8 | 103305906 | 146279416 | 0.3626 | 1.3034 | 3.9443 | 0.9919 | 4 | 1 |
| WCM63_Z19_2 | 9 | 116867 | 28888930 | −0.2032 | 0.5779 | 2.0615 | 0.924 | 2 | 1 |
| WCM63_Z19_2 | 9 | 32384732 | 38411484 | 0.1338 | 1.0195 | 2.0682 | 1.9861 | 2 | 2 |
| WCM63_Z19_2 | 9 | 38413346 | 141111414 | −0.2067 | 0.5732 | 2.0045 | 0.9711 | 2 | 1 |
| WCM63_Z19_2 | X | 2707682 | 22291762 | −0.6149 | −0.0166 | NA | NA | NA | NA |
| WCM63_Z19_2 | X | 22291972 | 27765205 | 0.0855 | 0.9581 | NA | NA | NA | NA |
| WCM63_Z19_2 | X | 27765657 | 100105218 | −0.584 | 0.0308 | NA | NA | NA | NA |
| WCM63_Z19_2 | X | 100106293 | 154509348 | 0.0616 | 0.9275 | NA | NA | NA | NA |
| WCM63_Z2_1 | 1 | 69590 | 120491704 | −0.2811 | −0.3973 | 1.0145 | 0.5041 | 1 | 1 |
| WCM63_Z2_1 | 1 | 120510178 | 249212557 | 0.0801 | −0.0095 | 0.9977 | 0.9892 | 1 | 1 |
| WCM63_Z2_1 | 10 | 226016 | 9281413 | −0.7952 | −0.9643 | 1.0278 | −0.0027 | 1 | 0 |
| WCM63_Z2_1 | 10 | 11047400 | 13167995 | −0.3225 | −0.4422 | 0.9607 | 0.5112 | 1 | 1 |
| WCM63_Z2_1 | 10 | 13169898 | 17641339 | −0.787 | −0.9551 | 1.0178 | 0.0138 | 1 | 0 |
| WCM63_Z2_1 | 10 | 17686365 | 23257397 | −0.357 | −0.4798 | 0.9272 | 0.507 | 1 | 1 |
| WCM63_Z2_1 | 10 | 23270419 | 28527518 | 0.0693 | −0.0209 | 1.0412 | 0.93 | 1 | 1 |
| WCM63_Z2_1 | 10 | 28822523 | 30336727 | −0.2485 | −0.362 | 0.9608 | 0.5954 | 1 | 1 |
| WCM63_Z2_1 | 10 | 30602783 | 35896538 | −0.6863 | −0.8424 | 1.0935 | 0.0219 | 1 | 0 |
| WCM63_Z2_1 | 10 | 35896983 | 56424006 | 0.045 | −0.0469 | 1 | 0.936 | 1 | 1 |
| WCM63_Z2_1 | 10 | 57359710 | 59064251 | −0.9505 | −1.1402 | NA | NA | NA | NA |
| WCM63_Z2_1 | 10 | 59956189 | 135440117 | 0.0743 | −0.0156 | 0.9903 | 0.9882 | 1 | 1 |
| WCM63_Z2_1 | 11 | 193144 | 5255444 | 0.1984 | 0.1161 | 1.2307 | 0.9369 | 1 | 1 |
| WCM63_Z2_1 | 11 | 5263449 | 40137779 | 0.0642 | −0.0264 | 0.9881 | 0.9756 | 1 | 1 |
| WCM63_Z2_1 | 11 | 42210168 | 49221910 | −0.8134 | −0.9848 | 0.9424 | 0.0682 | 1 | 0 |
| WCM63_Z2_1 | 11 | 49597890 | 107300165 | 0.0931 | 0.0044 | 1.0126 | 0.9934 | 1 | 1 |
| WCM63_Z2_1 | 11 | 107304361 | 126968472 | −0.8167 | −0.9884 | 1.0083 | −2.00E−04 | 1 | 0 |
| WCM63_Z2_1 | 11 | 126969289 | 134856562 | 0.0929 | 0.0041 | 1.0359 | 0.9698 | 1 | 1 |
| WCM63_Z2_1 | 12 | 148674 | 133810703 | 0.0831 | −0.0062 | 0.9999 | 0.9914 | 1 | 1 |
| WCM63_Z2_1 | 13 | 19600515 | 30829619 | −0.3055 | −0.4237 | 0.9373 | 0.5537 | 1 | 1 |
| WCM63_Z2_1 | 13 | 30854358 | 92101017 | −0.8846 | −1.0652 | 0.9682 | −0.0124 | 1 | 0 |
| WCM63_Z2_1 | 13 | 92345691 | 115089411 | −0.3406 | −0.4619 | 0.9425 | 0.5096 | 1 | 1 |
| WCM63_Z2_1 | 14 | 19377723 | 107283213 | 0.0634 | −0.0273 | 0.9876 | 0.975 | 1 | 1 |
| WCM63_Z2_1 | 15 | 20170150 | 102517666 | −0.2953 | −0.4127 | 1.0063 | 0.4961 | 1 | 0 |
| WCM63_Z2_1 | 16 | 66953 | 1251963 | 0.416 | 0.3456 | 2.0327 | 0.5087 | 2 | 1 |
| WCM63_Z2_1 | 16 | 1252259 | 33965573 | 0.6385 | 0.5784 | 1.982 | 1.0043 | 2 | 1 |
| WCM63_Z2_1 | 16 | 34256808 | 49672214 | 1.0441 | 0.999 | 2.9885 | 1.0086 | 3 | 1 |
| WCM63_Z2_1 | 16 | 49672520 | 53675301 | 0.0586 | −0.0324 | 1.0216 | 0.9341 | 1 | 1 |
| WCM63_Z2_1 | 16 | 53679551 | 69496362 | −0.8187 | −0.9907 | 1.0164 | −0.01 | 1 | 0 |
| WCM63_Z2_1 | 16 | 69602425 | 69788812 | 1.008 | 0.9618 | NA | NA | NA | NA |
| WCM63_Z2_1 | 16 | 69820970 | 73177676 | 0.1092 | 0.0215 | 1.0323 | 0.9977 | 1 | 1 |
| WCM63_Z2_1 | 16 | 73517324 | 90162135 | −0.7838 | −0.9515 | 1.0278 | 0.0065 | 1 | 0 |
| WCM63_Z2_1 | 17 | 131595 | 21188249 | −0.2707 | −0.386 | 1.0123 | 0.5182 | 1 | 1 |
| WCM63_Z2_1 | 17 | 21191346 | 25975908 | 0.0024 | −0.0923 | 1.0763 | 0.7998 | 1 | 1 |
| WCM63_Z2_1 | 17 | 25981805 | 47121328 | 0.1821 | 0.0989 | 1.0823 | 1.0596 | 1 | 1 |
| WCM63_Z2_1 | 17 | 47122370 | 81083507 | 0.037 | −0.0554 | 0.9767 | 0.948 | 1 | 1 |
| WCM63_Z2_1 | 18 | 48236 | 78005210 | 0.0489 | −0.0427 | 0.9832 | 0.9584 | 1 | 1 |
| WCM63_Z2_1 | 19 | 281535 | 59083951 | −0.2654 | −0.3803 | 1.0262 | 0.5103 | 1 | 1 |
| WCM63_Z2_1 | 2 | 41623 | 90199107 | −0.3065 | −0.4248 | 0.9843 | 0.5055 | 1 | 1 |
| WCM63_Z2_1 | 2 | 90229327 | 115919809 | 0.0633 | −0.0274 | 1.0042 | 0.9582 | 1 | 1 |
| WCM63_Z2_1 | 2 | 115991546 | 125671840 | −0.8239 | −0.9965 | 0.9996 | 0.0028 | 1 | 0 |
| WCM63_Z2_1 | 2 | 127413862 | 141641404 | −1.6692 | −1.9963 | 0.5029 | −0.0016 | 1 | 0 |
| WCM63_Z2_1 | 2 | 141660586 | 242946607 | −0.3253 | −0.4453 | 0.9707 | 0.4981 | 1 | 0 |
| WCM63_Z2_1 | 20 | 68396 | 23967150 | −0.3028 | −0.4209 | 0.9926 | 0.5013 | 1 | 1 |
| WCM63_Z2_1 | 20 | 24523899 | 30467693 | 0.3259 | 0.2508 | 1.2412 | 1.1384 | 1 | 1 |
| WCM63_Z2_1 | 20 | 30475042 | 32026712 | 0.6007 | 0.5389 | 1.9185 | 0.9872 | 2 | 1 |
| WCM63_Z2_1 | 20 | 32031205 | 37121690 | 0.2395 | 0.1596 | 1.1843 | 1.0497 | 1 | 1 |
| WCM63_Z2_1 | 20 | 37126150 | 62904847 | −0.2916 | −0.4087 | 0.9911 | 0.5155 | 1 | 1 |
| WCM63_Z2_1 | 21 | 9907291 | 48084282 | 0.1114 | 0.0238 | 1.0383 | 0.995 | 1 | 1 |
| WCM63_Z2_1 | 22 | 16287451 | 51215134 | −0.2511 | −0.3648 | 1.0228 | 0.5303 | 1 | 1 |
| WCM63_Z2_1 | 3 | 239390 | 24164472 | 0.3821 | 0.3099 | 1.4847 | 0.9946 | 1 | 1 |
| WCM63_Z2_1 | 3 | 24168987 | 49095364 | 0.0869 | −0.0022 | 1.0038 | 0.9931 | 1 | 1 |
| WCM63_Z2_1 | 3 | 49114160 | 49695542 | −0.2181 | −0.3291 | NA | NA | NA | NA |
| WCM63_Z2_1 | 3 | 49698091 | 89528638 | 0.082 | −0.0075 | 1.016 | 0.9737 | 1 | 1 |
| WCM63_Z2_1 | 3 | 93595842 | 132036298 | 0.6037 | 0.5421 | 1.9258 | 0.9864 | 2 | 1 |
| WCM63_Z2_1 | 3 | 132051167 | 132441155 | 1.278 | 1.2398 | NA | NA | NA | NA |
| WCM63_Z2_1 | 3 | 133098837 | 197894713 | 0.5941 | 0.5321 | 1.9035 | 0.9885 | 2 | 1 |
| WCM63_Z2_1 | 4 | 59331 | 49040138 | 0.0674 | −0.0229 | 0.9926 | 0.9758 | 1 | 1 |
| WCM63_Z2_1 | 4 | 49046800 | 82380654 | 1.274 | 1.2357 | 3.6998 | 1.0101 | 4 | 1 |
| WCM63_Z2_1 | 4 | 83276547 | 133367616 | 0.0293 | −0.0636 | 0.9712 | 0.9426 | 1 | 1 |
| WCM63_Z2_1 | 4 | 133368235 | 135121501 | −3.4541 | −5.1881 | NA | NA | NA | NA |
| WCM63_Z2_1 | 4 | 135121842 | 142949975 | −0.8319 | −1.0056 | 1.0049 | −0.0088 | 1 | 0 |
| WCM63_Z2_1 | 4 | 142951306 | 190426502 | 0.027 | −0.066 | 0.9685 | 0.942 | 1 | 1 |
| WCM63_Z2_1 | 5 | 140532 | 16067663 | 0.6083 | 0.5469 | 1.9259 | 0.9959 | 2 | 1 |
| WCM63_Z2_1 | 5 | 16067862 | 17275364 | 0.0369 | −0.0555 | NA | NA | NA | NA |
| WCM63_Z2_1 | 5 | 17275799 | 45695896 | 0.5996 | 0.5378 | 1.9458 | 0.9577 | 2 | 1 |
| WCM63_Z2_1 | 5 | 49698142 | 66492459 | −0.8603 | −1.0376 | 0.9758 | −0.0016 | 1 | 0 |
| WCM63_Z2_1 | 5 | 67494070 | 82373138 | 0.05 | −0.0416 | 0.9933 | 0.9499 | 1 | 1 |
| WCM63_Z2_1 | 5 | 82406983 | 110462504 | −0.8434 | −1.0186 | 0.9802 | 0.007 | 1 | 0 |
| WCM63_Z2_1 | 5 | 110560257 | 112043485 | −3.335 | −4.8248 | NA | NA | NA | NA |
| WCM63_Z2_1 | 5 | 112090601 | 130764860 | −0.8345 | −1.0085 | 0.9878 | 0.0063 | 1 | 0 |
| WCM63_Z2_1 | 5 | 130766415 | 180687808 | 0.0721 | −0.018 | 0.9928 | 0.9823 | 1 | 1 |
| WCM63_Z2_1 | 6 | 203516 | 12296255 | −0.2763 | −0.392 | 0.9784 | 0.5457 | 1 | 1 |
| WCM63_Z2_1 | 6 | 12934068 | 18572091 | 0.1849 | 0.1018 | 1.114 | 1.0322 | 1 | 1 |
| WCM63_Z2_1 | 6 | 19838288 | 56323827 | −0.2829 | −0.3992 | 1.0032 | 0.5133 | 1 | 1 |
| WCM63_Z2_1 | 6 | 56324990 | 66205172 | 0.0352 | −0.0573 | 0.9857 | 0.9365 | 1 | 1 |
| WCM63_Z2_1 | 6 | 68598997 | 84573919 | −0.3888 | −0.5144 | 0.9499 | 0.4503 | 1 | 0 |
| WCM63_Z2_1 | 6 | 84618756 | 88331194 | −0.8149 | −0.9864 | NA | NA | NA | NA |
| WCM63_Z2_1 | 6 | 88346168 | 119238820 | −0.3156 | −0.4347 | 0.9456 | 0.5341 | 1 | 1 |
| WCM63_Z2_1 | 6 | 119243243 | 119399331 | −1.5824 | −1.8881 | NA | NA | NA | NA |
| WCM63_Z2_1 | 6 | 119501086 | 170893437 | −0.3255 | −0.4455 | 0.9435 | 0.5252 | 1 | 1 |
| WCM63_Z2_1 | 7 | 193222 | 64254947 | 0.6135 | 0.5523 | 1.9836 | 0.9492 | 2 | 1 |
| WCM63_Z2_1 | 7 | 64275300 | 158937416 | 0.0605 | −0.0303 | 1.0689 | 0.8895 | 1 | 1 |
| WCM63_Z2_1 | 8 | 190906 | 37456103 | −0.8111 | −0.9821 | 1.0124 | 0 | 1 | 0 |
| WCM63_Z2_1 | 8 | 37553685 | 80523432 | 0.6602 | 0.601 | 1.9973 | 1.0363 | 2 | 1 |
| WCM63_Z2_1 | 8 | 80549097 | 126194478 | 0.4327 | 0.3631 | 1.4914 | 1.081 | 1 | 1 |
| WCM63_Z2_1 | 8 | 126369507 | 146279511 | 0.1865 | 0.1035 | 1.5604 | 0.5883 | 2 | 1 |
| WCM63_Z2_1 | 9 | 116887 | 141111414 | 0.0788 | −0.0109 | 0.9999 | 0.9851 | 1 | 1 |
| WCM63_Z2_1 | X | 2707716 | 46309907 | −0.8601 | −1.0375 | NA | NA | NA | NA |
| WCM63_Z2_1 | X | 46322675 | 154509348 | 0.5647 | 0.5013 | NA | NA | NA | NA |
| WCM677 | 1 | 14907 | 5924359 | 0.1387 | 0.1862 | 1.1519 | 1.1236 | 1 | 1 |
| WCM677 | 1 | 5924444 | 16455987 | −0.3927 | −0.9602 | 1.0169 | 0.0111 | 1 | 0 |
| WCM677 | 1 | 16456656 | 18024350 | −0.3157 | −0.7593 | 0.9173 | 0.2642 | 1 | 0 |
| WCM677 | 1 | 18149510 | 18962716 | −0.3886 | −0.949 | NA | NA | NA | NA |
| WCM677 | 1 | 18965426 | 145457631 | 0.0368 | −4.00E−04 | 0.9997 | 0.9997 | 1 | 1 |
| WCM677 | 1 | 145457926 | 156623141 | 0.3975 | 0.6188 | 1.9329 | 1.1383 | 2 | 1 |
| WCM677 | 1 | 156626132 | 160264666 | 0.0778 | 0.076 | 1.1085 | 0.9998 | 1 | 1 |
| WCM677 | 1 | 160265672 | 249213464 | 0.3888 | 0.6051 | 1.9184 | 1.1238 | 2 | 1 |
| WCM677 | 10 | 226104 | 135438994 | 0.0333 | −0.0069 | 0.9952 | 0.9952 | 1 | 1 |
| WCM677 | 11 | 193096 | 103814416 | 0.0434 | 0.0121 | 1.0198 | 0.9971 | 1 | 1 |
| WCM677 | 11 | 103818411 | 126870418 | −0.4221 | −1.0416 | 0.9525 | 0.0191 | 1 | 0 |
| WCM677 | 11 | 126873592 | 134605906 | 0.0162 | −0.0396 | 0.999 | 0.9469 | 1 | 1 |
| WCM677 | 12 | 148388 | 9748018 | 0.0113 | −0.0489 | 1.0056 | 0.9277 | 1 | 1 |
| WCM677 | 12 | 9750669 | 15834157 | −0.433 | −1.0727 | 0.9604 | −0.0095 | 1 | 0 |
| WCM677 | 12 | 15835855 | 133810869 | 0.0392 | 0.0042 | 1.0029 | 1.0029 | 1 | 1 |
| WCM677 | 13 | 19042004 | 41032853 | 0.005 | −0.0611 | 0.9586 | 0.9586 | 1 | 1 |
| WCM677 | 13 | 41034618 | 70535607 | −0.4914 | −1.2469 | 0.9145 | −0.0718 | 1 | 0 |
| WCM677 | 13 | 70549818 | 115089411 | 0.0045 | −0.062 | 0.9579 | 0.9579 | 1 | 1 |
| WCM677 | 14 | 19110194 | 107283270 | 0.0432 | 0.0118 | 1.0082 | 1.0082 | 1 | 1 |
| WCM677 | 15 | 20083896 | 102514214 | −0.1726 | −0.4249 | 0.9211 | 0.5687 | 1 | 1 |
| WCM677 | 16 | 64396 | 25712440 | 0.0555 | 0.0346 | 1.0511 | 0.9976 | 1 | 1 |
| WCM677 | 16 | 25719928 | 32402223 | −0.5158 | −1.324 | 0.8419 | −0.043 | 1 | 0 |
| WCM677 | 16 | 32416429 | 48675332 | −0.0371 | −0.1431 | 1.2623 | 0.5489 | 1 | 1 |
| WCM677 | 16 | 48686063 | 61935280 | −0.4145 | −1.0204 | 0.9883 | −0.0023 | 1 | 0 |
| WCM677 | 16 | 62055230 | 70177614 | 0.0604 | 0.0439 | 1.075 | 0.9868 | 1 | 1 |
| WCM677 | 16 | 70180141 | 90260461 | −0.4092 | −1.0055 | 0.969 | 0.0272 | 1 | 0 |
| WCM677 | 17 | 131595 | 81187813 | 0.0394 | 0.0046 | 1.0032 | 1.0032 | 1 | 1 |
| WCM677 | 18 | 47661 | 78005110 | 0.0086 | −0.054 | 1.9179 | 0.0086 | 2 | 0 |
| WCM677 | 19 | 105050 | 59093645 | 0.0208 | −0.0308 | 0.9789 | 0.9789 | 1 | 1 |
| WCM677 | 2 | 41612 | 116601843 | 0.0151 | −0.0416 | 0.9716 | 0.9716 | 1 | 1 |
| WCM677 | 2 | 118572355 | 148657339 | −0.4584 | −1.1466 | 0.8894 | 0.014 | 1 | 0 |
| WCM677 | 2 | 148657490 | 162567121 | 0.004 | −0.0628 | 0.9666 | 0.9482 | 1 | 1 |
| WCM677 | 2 | 162627444 | 189447071 | −0.2264 | −0.5455 | 0.7606 | 0.6097 | 1 | 1 |
| WCM677 | 2 | 189447335 | 218577646 | −0.0021 | −0.0747 | 0.9495 | 0.9495 | 1 | 1 |
| WCM677 | 2 | 218604181 | 242814884 | −0.4191 | −1.0334 | 0.9562 | 0.0209 | 1 | 0 |
| WCM677 | 2 | 242815059 | 243037013 | −0.4061 | −0.997 | NA | NA | NA | NA |
| WCM677 | 20 | 68299 | 62934617 | −0.1562 | −0.3894 | 1.0308 | 0.4961 | 1 | 0 |
| WCM677 | 21 | 9590311 | 48084175 | 0.0371 | 1.00E−04 | 1.0001 | 1.0001 | 1 | 1 |
| WCM677 | 22 | 16159014 | 51219089 | −0.3764 | −0.9161 | 1.0485 | 0.0114 | 1 | 0 |
| WCM677 | 3 | 239436 | 97634601 | −0.0818 | −0.233 | 0.87 | 0.8317 | 1 | 1 |
| WCM677 | 3 | 97643140 | 118913289 | −0.4474 | −1.1143 | 0.9029 | 0.0209 | 1 | 0 |
| WCM677 | 3 | 118917943 | 197896801 | 0.2851 | 0.4372 | 1.596 | 1.112 | 2 | 1 |
| WCM677 | 4 | 53344 | 117220900 | 0.012 | −0.0476 | 0.9676 | 0.9676 | 1 | 1 |
| WCM677 | 4 | 118004865 | 138149449 | −0.5255 | −1.3555 | 0.8495 | −0.0678 | 1 | 0 |
| WCM677 | 4 | 138440988 | 190904482 | −0.0182 | −0.1061 | 0.9291 | 0.9291 | 1 | 1 |
| WCM677 | 5 | 90576 | 1443182 | −0.0028 | −0.0759 | 1.2369 | 0.6605 | 1 | 1 |
| WCM677 | 5 | 1461779 | 26906284 | −0.2205 | −0.5321 | 0.8683 | 0.5148 | 1 | 1 |
| WCM677 | 5 | 26906844 | 180687830 | 0.0272 | −0.0185 | 0.9873 | 0.9873 | 1 | 1 |
| WCM677 | 6 | 203588 | 80411756 | −0.1962 | −0.4772 | 0.9784 | 0.4583 | 1 | 0 |
| WCM677 | 6 | 80412040 | 93967201 | −0.4928 | −1.2513 | 0.9075 | −0.0674 | 1 | 0 |
| WCM677 | 6 | 93967851 | 170892873 | −0.2247 | −0.5417 | 0.9419 | 0.432 | 1 | 0 |
| WCM677 | 7 | 193245 | 5545157 | 0.1547 | 0.2146 | 1.2096 | 1.1113 | 1 | 1 |
| WCM677 | 7 | 5553263 | 127231302 | 5.00E−04 | −0.0696 | 0.9713 | 0.9345 | 1 | 1 |
| WCM677 | 7 | 127231699 | 158937074 | 0.0696 | 0.061 | 1.0663 | 1.02 | 1 | 1 |
| WCM677 | 8 | 163438 | 43415878 | −0.1982 | −0.4817 | 1.4454 | −0.0132 | 1 | 0 |
| WCM677 | 8 | 47886108 | 51705393 | 0.9305 | 1.3886 | NA | NA | NA | NA |
| WCM677 | 8 | 52232487 | 65496220 | 0.6003 | 0.927 | 3.7721 | 0.0305 | 4 | 0 |
| WCM677 | 8 | 65509349 | 146279472 | 0.9945 | 1.4736 | 3.9497 | 1.6047 | 4 | 2 |
| WCM677 | 9 | 14903 | 103348297 | 0.0128 | −0.0459 | 0.9687 | 0.9687 | 1 | 1 |
| WCM677 | 9 | 103348634 | 112711585 | −0.4354 | −1.0796 | 0.9723 | −0.0259 | 1 | 0 |
| WCM677 | 9 | 112713081 | 141107526 | 0.1174 | 0.1482 | 1.1082 | 1.1082 | 1 | 1 |
| WCM677 | X | 2700087 | 154493609 | −0.0944 | −0.2588 | NA | NA | NA | NA |
| WCM677_Z12_1 | 1 | 721476 | 3809495 | 0.0788 | 0.0351 | 1.0402 | 1.0091 | 1 | 1 |
| WCM677_Z12_1 | 1 | 3816745 | 18962771 | −0.8752 | −0.9322 | 1.0475 | 6.00E−04 | 1 | 0 |
| WCM677_Z12_1 | 1 | 19018405 | 55538462 | 0.0897 | 0.0461 | 1.0325 | 1.0325 | 1 | 1 |
| WCM677_Z12_1 | 1 | 55539559 | 109649631 | −0.033 | −0.0778 | 0.96 | 0.9351 | 1 | 1 |
| WCM677_Z12_1 | 1 | 109650634 | 145109583 | 0.0585 | 0.0146 | 1.0459 | 0.9745 | 1 | 1 |
| WCM677_Z12_1 | 1 | 145112393 | 156568810 | 0.6574 | 0.6185 | 2.0498 | 1.0206 | 2 | 1 |
| WCM677_Z12_1 | 1 | 156571224 | 160254843 | 0.061 | 0.0172 | 1.0294 | 0.9946 | 1 | 1 |
| WCM677_Z12_1 | 1 | 160259952 | 249212424 | 0.6143 | 0.575 | 1.9771 | 1.0023 | 2 | 1 |
| WCM677_Z12_1 | 10 | 225981 | 135440105 | 0.0408 | −0.0033 | 0.9977 | 0.9977 | 1 | 1 |
| WCM677_Z12_1 | 11 | 193105 | 103720698 | 0.0678 | 0.024 | 1.0168 | 1.0168 | 1 | 1 |
| WCM677_Z12_1 | 11 | 103780503 | 126969252 | −0.8998 | −0.9573 | 1.0296 | 4.00E−04 | 1 | 0 |
| WCM677_Z12_1 | 11 | 128332328 | 134856563 | 0.0708 | 0.0271 | 1.0324 | 1.0056 | 1 | 1 |
| WCM677_Z12_1 | 12 | 148639 | 7061279 | 0.0958 | 0.0523 | 1.0683 | 1.0055 | 1 | 1 |
| WCM677_Z12_1 | 12 | 7064121 | 7633746 | −0.1005 | −0.146 | NA | NA | NA | NA |
| WCM677_Z12_1 | 12 | 7635291 | 8090973 | −0.9807 | −1.0399 | NA | NA | NA | NA |
| WCM677_Z12_1 | 12 | 8192452 | 8925987 | 0.0478 | 0.0038 | NA | NA | NA | NA |
| WCM677_Z12_1 | 12 | 8930225 | 9087059 | −0.9416 | −1 | NA | NA | NA | NA |
| WCM677_Z12_1 | 12 | 9087727 | 9465276 | 0.0099 | −0.0345 | NA | NA | NA | NA |
| WCM677_Z12_1 | 12 | 9571404 | 15835885 | −0.9617 | −1.0204 | 0.991 | −0.0051 | 1 | 0 |
| WCM677_Z12_1 | 12 | 16035709 | 59313789 | 0.0661 | 0.0223 | 1.0248 | 1.0063 | 1 | 1 |
| WCM677_Z12_1 | 12 | 59917206 | 103670968 | −0.0301 | −0.0749 | 0.9619 | 0.9369 | 1 | 1 |
| WCM677_Z12_1 | 12 | 103695973 | 133810896 | 0.0699 | 0.0262 | 1.0224 | 1.0142 | 1 | 1 |
| WCM677_Z12_1 | 13 | 19600515 | 40362474 | 0.0066 | −0.0378 | 0.987 | 0.9614 | 1 | 1 |
| WCM677_Z12_1 | 13 | 41133777 | 70549835 | −0.9596 | −1.0184 | 0.9874 | 0 | 1 | 0 |
| WCM677_Z12_1 | 13 | 70681400 | 115089411 | 0.0107 | −0.0336 | 1.0066 | 0.9474 | 1 | 1 |
| WCM677_Z12_1 | 14 | 19377712 | 107283068 | 0.0429 | −0.0011 | 0.9992 | 0.9992 | 1 | 1 |
| WCM677_Z12_1 | 15 | 20170094 | 24922202 | −0.3099 | −0.3579 | 0.8554 | 0.7052 | 1 | 1 |
| WCM677_Z12_1 | 15 | 24922401 | 102517659 | −0.3641 | −0.4128 | 1.0001 | 0.5022 | 1 | 1 |
| WCM677_Z12_1 | 16 | 67604 | 26147480 | 0.0803 | 0.0367 | 1.032 | 1.0196 | 1 | 1 |
| WCM677_Z12_1 | 16 | 27078221 | 32013471 | −0.899 | −0.9565 | 1.0281 | 0.0025 | 1 | 0 |
| WCM677_Z12_1 | 16 | 32077496 | 48643772 | −0.0066 | −0.0511 | 0.9993 | 0.931 | 1 | 1 |
| WCM677_Z12_1 | 16 | 49193569 | 61935359 | −0.9052 | −0.9628 | 1.0257 | 4.00E−04 | 1 | 0 |
| WCM677_Z12_1 | 16 | 62055118 | 70191029 | 0.082 | 0.0383 | 1.0601 | 0.9937 | 1 | 1 |
| WCM677_Z12_1 | 16 | 70211274 | 90233642 | −0.9144 | −0.9722 | 1.0192 | 3.00E−04 | 1 | 0 |
| WCM677_Z12_1 | 17 | 131595 | 81083567 | 0.071 | 0.0272 | 1.0191 | 1.0191 | 1 | 1 |
| WCM677_Z12_1 | 18 | 48236 | 78005164 | 0.0178 | −0.0265 | 1.9638 | −2.00E−04 | 2 | 0 |
| WCM677_Z12_1 | 19 | 281506 | 59083985 | 0.0703 | 0.0265 | 1.0186 | 1.0186 | 1 | 1 |
| WCM677_Z12_1 | 2 | 41623 | 11802360 | 0.0136 | −0.0307 | 0.9958 | 0.9622 | 1 | 1 |
| WCM677_Z12_1 | 2 | 11809731 | 11853913 | −0.9499 | −1.0084 | NA | NA | NA | NA |
| WCM677_Z12_1 | 2 | 11905676 | 116599858 | 0.0241 | −0.0201 | 0.9912 | 0.9812 | 1 | 1 |
| WCM677_Z12_1 | 2 | 118572384 | 144765060 | −0.9324 | −0.9906 | 1.0064 | 2.00E−04 | 1 | 0 |
| WCM677_Z12_1 | 2 | 144899623 | 145274873 | −0.8509 | −0.9075 | NA | NA | NA | NA |
| WCM677_Z12_1 | 2 | 147345768 | 162274748 | 0.0221 | −0.0221 | 1.0137 | 0.956 | 1 | 1 |
| WCM677_Z12_1 | 2 | 162275474 | 188332522 | −0.4062 | −0.4555 | 0.9629 | 0.4956 | 1 | 0 |
| WCM677_Z12_1 | 2 | 188343519 | 207814400 | −0.0099 | −0.0545 | 0.9736 | 0.9522 | 1 | 1 |
| WCM677_Z12_1 | 2 | 207820276 | 208485372 | −1.0382 | −1.0986 | NA | NA | NA | NA |
| WCM677_Z12_1 | 2 | 208486581 | 218604265 | 0.0267 | −0.0175 | 1.0109 | 0.965 | 1 | 1 |
| WCM677_Z12_1 | 2 | 218620846 | 242946597 | −0.9099 | −0.9677 | 1.0223 | 3.00E−04 | 1 | 0 |
| WCM677_Z12_1 | 20 | 68406 | 33080396 | −0.3338 | −0.3821 | 1.0254 | 0.5092 | 1 | 1 |
| WCM677_Z12_1 | 20 | 33092196 | 33442624 | −1.5882 | −1.6632 | NA | NA | NA | NA |
| WCM677_Z12_1 | 20 | 33444677 | 49508567 | −0.2335 | −0.2806 | 1.0875 | 0.5591 | 1 | 1 |
| WCM677_Z12_1 | 20 | 49509045 | 62904932 | −0.3449 | −0.3934 | 1.0113 | 0.5114 | 1 | 1 |
| WCM677_Z12_1 | 21 | 9907284 | 48084253 | 0.0421 | −0.0019 | 1.0118 | 0.9856 | 1 | 1 |
| WCM677_Z12_1 | 22 | 16287438 | 51215152 | −0.8702 | −0.9271 | 1.0565 | −0.0047 | 1 | 0 |
| WCM677_Z12_1 | 3 | 239347 | 89462368 | −0.3454 | −0.3939 | 1.021 | 0.5012 | 1 | 1 |
| WCM677_Z12_1 | 3 | 89468375 | 97655642 | 0.1867 | 0.1441 | NA | NA | NA | NA |
| WCM677_Z12_1 | 3 | 97660104 | 118878762 | −0.9723 | −1.0313 | 0.9781 | 4.00E−04 | 1 | 0 |
| WCM677_Z12_1 | 3 | 118905598 | 197894726 | 0.3397 | 0.2984 | 1.4703 | 0.9892 | 1 | 1 |
| WCM677_Z12_1 | 4 | 59448 | 117220900 | 0.0079 | −0.0365 | 0.9819 | 0.9681 | 1 | 1 |
| WCM677_Z12_1 | 4 | 118005455 | 135122163 | −0.9915 | −1.0509 | 0.9631 | 0.0022 | 1 | 0 |
| WCM677_Z12_1 | 4 | 138442430 | 190426502 | −0.0135 | −0.0581 | 0.9702 | 0.9508 | 1 | 1 |
| WCM677_Z12_1 | 5 | 140684 | 24535261 | −0.3359 | −0.3843 | 0.985 | 0.5472 | 1 | 1 |
| WCM677_Z12_1 | 5 | 24535917 | 180687773 | 0.029 | −0.0152 | 0.9895 | 0.9895 | 1 | 1 |
| WCM677_Z12_1 | 6 | 203466 | 80383517 | −0.3638 | −0.4126 | 1.0078 | 0.4947 | 1 | 0 |
| WCM677_Z12_1 | 6 | 80406233 | 93953228 | −0.9481 | −1.0066 | 0.9929 | 0.0026 | 1 | 0 |
| WCM677_Z12_1 | 6 | 93955019 | 170893415 | −0.4042 | −0.4535 | 0.9923 | 0.4682 | 1 | 0 |
| WCM677_Z12_1 | 7 | 193254 | 158937416 | 0.035 | −0.0091 | 0.9997 | 0.9877 | 1 | 1 |
| WCM677_Z12_1 | 8 | 190906 | 43212026 | −0.36 | −0.4087 | 1.5091 | −0.0025 | 2 | 0 |
| WCM677_Z12_1 | 8 | 47752739 | 51465616 | 1.3758 | 1.3406 | NA | NA | NA | NA |
| WCM677_Z12_1 | 8 | 51571163 | 65493792 | 0.9672 | 0.9301 | 3.8017 | 0.0091 | 4 | 0 |
| WCM677_Z12_1 | 8 | 65494220 | 146279508 | 1.4489 | 1.414 | 3.855 | 1.4744 | 4 | 1 |
| WCM677_Z12_1 | 9 | 17055 | 8733822 | −0.0054 | −0.0499 | 0.9962 | 0.9357 | 1 | 1 |
| WCM677_Z12_1 | 9 | 12694196 | 14102457 | −1.0131 | −1.073 | NA | NA | NA | NA |
| WCM677_Z12_1 | 9 | 14113017 | 103348634 | 0.032 | −0.0121 | 1.0027 | 0.9806 | 1 | 1 |
| WCM677_Z12_1 | 9 | 104032203 | 112969805 | −0.9519 | −1.0105 | 0.9927 | 0 | 1 | 0 |
| WCM677_Z12_1 | 9 | 113006466 | 141111414 | 0.0897 | 0.0462 | 1.0325 | 1.0325 | 1 | 1 |
| WCM677_Z12_1 | X | 2707734 | 154509348 | −0.0086 | −0.0532 | NA | NA | NA | NA |
| WCM677_Z16_1 | 1 | 721476 | 3809556 | 0.0839 | 0.0411 | 1.0405 | 1.0172 | 1 | 1 |
| WCM677_Z16_1 | 1 | 3816750 | 19018405 | −0.8529 | −0.9359 | 1.0426 | 0.0028 | 1 | 0 |
| WCM677_Z16_1 | 1 | 19027206 | 55551698 | 0.0831 | 0.0403 | 1.0283 | 1.0283 | 1 | 1 |
| WCM677_Z16_1 | 1 | 55555377 | 115537573 | −0.0084 | −0.0541 | 0.9732 | 0.9533 | 1 | 1 |
| WCM677_Z16_1 | 1 | 115576128 | 145209360 | 0.0716 | 0.0284 | 1.229 | 0.8108 | 1 | 1 |
| WCM677_Z16_1 | 1 | 145281644 | 156568810 | 0.6546 | 0.6259 | 2.072 | 1.0143 | 2 | 1 |
| WCM677_Z16_1 | 1 | 156571231 | 160254880 | 0.0648 | 0.0214 | 1.0651 | 0.9647 | 1 | 1 |
| WCM677_Z16_1 | 1 | 160259961 | 249212369 | 0.6016 | 0.5718 | 2.0007 | 0.972 | 2 | 1 |
| WCM677_Z16_1 | 10 | 225982 | 135440090 | 0.0468 | 0.0029 | 1.002 | 1.002 | 1 | 1 |
| WCM677_Z16_1 | 11 | 193152 | 103327019 | 0.0702 | 0.027 | 1.0252 | 1.0126 | 1 | 1 |
| WCM677_Z16_1 | 11 | 103339332 | 126969249 | −0.8867 | −0.9717 | 1.0178 | 0.0021 | 1 | 0 |
| WCM677_Z16_1 | 11 | 128332268 | 134856563 | 0.0611 | 0.0176 | 1.0321 | 0.9925 | 1 | 1 |
| WCM677_Z16_1 | 12 | 148674 | 7632561 | 0.0786 | 0.0356 | 1.0345 | 1.0155 | 1 | 1 |
| WCM677_Z16_1 | 12 | 7633843 | 8192567 | −0.9475 | −1.0363 | NA | NA | NA | NA |
| WCM677_Z16_1 | 12 | 8192707 | 9462943 | −0.0417 | −0.0884 | 1.1183 | 0.7628 | 1 | 1 |
| WCM677_Z16_1 | 12 | 9465477 | 15835866 | −0.9165 | −1.0033 | 1.0117 | −0.014 | 1 | 0 |
| WCM677_Z16_1 | 12 | 16035708 | 133810703 | 0.054 | 0.0103 | 1.0113 | 1.003 | 1 | 1 |
| WCM677_Z16_1 | 13 | 19600515 | 40362489 | −0.0071 | −0.0527 | 0.9751 | 0.9532 | 1 | 1 |
| WCM677_Z16_1 | 13 | 41133852 | 70535554 | −0.9324 | −1.0202 | 0.9982 | −0.0121 | 1 | 0 |
| WCM677_Z16_1 | 13 | 70549886 | 115089411 | 0.0046 | −0.0407 | 0.9863 | 0.9582 | 1 | 1 |
| WCM677_Z16_1 | 14 | 19377718 | 107283047 | 0.0409 | −0.0031 | 0.9978 | 0.9978 | 1 | 1 |
| WCM677_Z16_1 | 15 | 20170161 | 102517646 | −0.3452 | −0.403 | 1.0105 | 0.502 | 1 | 1 |
| WCM677_Z16_1 | 16 | 67604 | 26147482 | 0.0653 | 0.022 | 1.0216 | 1.0091 | 1 | 1 |
| WCM677_Z16_1 | 16 | 27078282 | 32077460 | −0.8848 | −0.9696 | 1.0181 | 0.0032 | 1 | 0 |
| WCM677_Z16_1 | 16 | 32486465 | 48643776 | −0.0115 | −0.0573 | 0.9988 | 0.9234 | 1 | 1 |
| WCM677_Z16_1 | 16 | 49193606 | 61935339 | −0.8763 | −0.9606 | 1.0232 | 0.0045 | 1 | 0 |
| WCM677_Z16_1 | 16 | 62055298 | 70191029 | 0.0609 | 0.0175 | 1.0445 | 0.9798 | 1 | 1 |
| WCM677_Z16_1 | 16 | 70211267 | 90233642 | −0.8956 | −0.9812 | 1.0064 | 0.0067 | 1 | 0 |
| WCM677_Z16_1 | 17 | 131595 | 81083561 | 0.0684 | 0.0252 | 1.0176 | 1.0176 | 1 | 1 |
| WCM677_Z16_1 | 18 | 48245 | 78005214 | 0.0159 | −0.029 | 1.9608 | −6.00E−04 | 2 | 0 |
| WCM677_Z16_1 | 19 | 281468 | 59083988 | 0.07 | 0.0268 | 1.0188 | 1.0188 | 1 | 1 |
| WCM677_Z16_1 | 2 | 41613 | 116599874 | 0.0384 | −0.0058 | 0.996 | 0.996 | 1 | 1 |
| WCM677_Z16_1 | 2 | 118572386 | 144765043 | −0.9213 | −1.0084 | 1.0082 | −0.014 | 1 | 0 |
| WCM677_Z16_1 | 2 | 144899623 | 147345771 | −0.8197 | −0.9007 | NA | NA | NA | NA |
| WCM677_Z16_1 | 2 | 147345885 | 162274748 | 0.0458 | 0.0019 | 1.0178 | 0.9848 | 1 | 1 |
| WCM677_Z16_1 | 2 | 162275518 | 188343531 | −0.4046 | −0.465 | 0.9602 | 0.4888 | 1 | 0 |
| WCM677_Z16_1 | 2 | 188349537 | 218604289 | −0.0063 | −0.0519 | 0.9756 | 0.9537 | 1 | 1 |
| WCM677_Z16_1 | 2 | 218620799 | 242946616 | −0.8829 | −0.9677 | 1.0259 | −0.0033 | 1 | 0 |
| WCM677_Z16_1 | 20 | 68363 | 33068922 | −0.3396 | −0.3973 | 1.009 | 0.5096 | 1 | 1 |
| WCM677_Z16_1 | 20 | 33077167 | 33433156 | −1.5817 | −1.7219 | NA | NA | NA | NA |
| WCM677_Z16_1 | 20 | 33437829 | 62904866 | −0.2791 | −0.3344 | 1.0618 | 0.5245 | 1 | 1 |
| WCM677_Z16_1 | 21 | 9907297 | 48084277 | 0.039 | −0.0051 | 1.0067 | 0.9862 | 1 | 1 |
| WCM677_Z16_1 | 22 | 16287440 | 51215123 | −0.8491 | −0.9319 | 1.0515 | −0.0032 | 1 | 0 |
| WCM677_Z16_1 | 3 | 239347 | 89521623 | −0.3387 | −0.3964 | 1.0152 | 0.5044 | 1 | 1 |
| WCM677_Z16_1 | 3 | 89528647 | 97634486 | 0.3279 | 0.2918 | NA | NA | NA | NA |
| WCM677_Z16_1 | 3 | 97655712 | 118878762 | −0.9479 | −1.0367 | 0.9872 | −0.0123 | 1 | 0 |
| WCM677_Z16_1 | 3 | 118905654 | 197894720 | 0.3353 | 0.2995 | 1.4653 | 0.996 | 1 | 1 |
| WCM677_Z16_1 | 4 | 59326 | 117220891 | −0.0018 | −0.0472 | 0.9728 | 0.9628 | 1 | 1 |
| WCM677_Z16_1 | 4 | 118005534 | 135122230 | −0.98 | −1.0708 | 0.9572 | −0.0051 | 1 | 0 |
| WCM677_Z16_1 | 4 | 138442299 | 190426502 | −0.0157 | −0.0615 | 0.9671 | 0.9493 | 1 | 1 |
| WCM677_Z16_1 | 5 | 140574 | 24537589 | −0.3561 | −0.4144 | 0.9824 | 0.5183 | 1 | 1 |
| WCM677_Z16_1 | 5 | 24593417 | 180687808 | 0.0379 | −0.0063 | 0.9957 | 0.9957 | 1 | 1 |
| WCM677_Z16_1 | 6 | 203479 | 80382863 | −0.3564 | −0.4148 | 0.9985 | 0.5018 | 1 | 1 |
| WCM677_Z16_1 | 6 | 80383505 | 92231446 | −0.9321 | −1.0199 | 0.9942 | −0.0079 | 1 | 0 |
| WCM677_Z16_1 | 6 | 93953208 | 170893450 | −0.3874 | −0.447 | 0.9797 | 0.4874 | 1 | 0 |
| WCM677_Z16_1 | 7 | 193205 | 158937375 | 0.0405 | −0.0036 | 1.0016 | 0.9934 | 1 | 1 |
| WCM677_Z16_1 | 8 | 190906 | 43211989 | −0.3484 | −0.4064 | 1.5085 | 5.00E−04 | 2 | 0 |
| WCM677_Z16_1 | 8 | 47752730 | 51465616 | 1.3941 | 1.377 | NA | NA | NA | NA |
| WCM677_Z16_1 | 8 | 51571204 | 65291793 | 0.9578 | 0.9345 | 3.8146 | 0.0079 | 4 | 0 |
| WCM677_Z16_1 | 8 | 65489068 | 146279511 | 1.4328 | 1.4162 | 3.8971 | 1.4404 | 4 | 1 |
| WCM677_Z16_1 | 9 | 17086 | 8733822 | −0.0048 | −0.0503 | 0.9757 | 0.9558 | 1 | 1 |
| WCM677_Z16_1 | 9 | 12694040 | 14088211 | −0.8624 | −0.9459 | NA | NA | NA | NA |
| WCM677_Z16_1 | 9 | 14102437 | 103348634 | 0.0374 | −0.0068 | 1.0035 | 0.9871 | 1 | 1 |
| WCM677_Z16_1 | 9 | 104032275 | 112969691 | −0.8876 | −0.9727 | 0.9949 | 0.0242 | 1 | 0 |
| WCM677_Z16_1 | 9 | 113006477 | 141111414 | 0.0857 | 0.043 | 1.0345 | 1.026 | 1 | 1 |
| WCM677_Z16_1 | X | 2707711 | 154509348 | −0.0164 | −0.0623 | NA | NA | NA | NA |
| WCM677_Z3_1 | 1 | 721415 | 3809482 | 0.0784 | 0.0509 | 1.0519 | 1.0199 | 1 | 1 |
| WCM677_Z3_1 | 1 | 3816745 | 18962795 | −0.8736 | −0.9419 | 1.0439 | −0.0028 | 1 | 0 |
| WCM677_Z3_1 | 1 | 19018405 | 65852599 | 0.0511 | 0.0227 | 1.018 | 1.0138 | 1 | 1 |
| WCM677_Z3_1 | 1 | 65854024 | 109294896 | −0.0478 | −0.0793 | 0.9632 | 0.9299 | 1 | 1 |
| WCM677_Z3_1 | 1 | 109295707 | 145209367 | 0.0198 | −0.0095 | 1.0938 | 0.893 | 1 | 1 |
| WCM677_Z3_1 | 1 | 145281677 | 156568810 | 0.6129 | 0.5987 | 1.9841 | 1.0446 | 2 | 1 |
| WCM677_Z3_1 | 1 | 156571231 | 160254854 | 0.0136 | −0.0158 | 1.0227 | 0.9554 | 1 | 1 |
| WCM677_Z3_1 | 1 | 160259952 | 249212366 | 0.577 | 0.562 | 1.9557 | 0.997 | 2 | 1 |
| WCM677_Z3_1 | 10 | 225980 | 135440080 | 0.0295 | 5.00E−04 | 1.0086 | 0.9921 | 1 | 1 |
| WCM677_Z3_1 | 11 | 193114 | 103720705 | 0.0273 | −0.0018 | 1.006 | 0.9916 | 1 | 1 |
| WCM677_Z3_1 | 11 | 103780446 | 126969303 | −0.8987 | −0.9685 | 1.0253 | −0.0033 | 1 | 0 |
| WCM677_Z3_1 | 11 | 128332323 | 134856556 | 0.0592 | 0.0311 | 1.0945 | 0.9492 | 1 | 1 |
| WCM677_Z3_1 | 12 | 148637 | 7083589 | 0.0636 | 0.0357 | 1.0694 | 0.9807 | 1 | 1 |
| WCM677_Z3_1 | 12 | 7169203 | 7633645 | −0.076 | −0.1084 | NA | NA | NA | NA |
| WCM677_Z3_1 | 12 | 7635307 | 8090972 | −0.9211 | −0.9923 | NA | NA | NA | NA |
| WCM677_Z3_1 | 12 | 8192529 | 8926151 | 0.0886 | 0.0614 | NA | NA | NA | NA |
| WCM677_Z3_1 | 12 | 8930196 | 9087060 | −0.9105 | −0.981 | NA | NA | NA | NA |
| WCM677_Z3_1 | 12 | 9087720 | 9355174 | 0.1525 | 0.1272 | NA | NA | NA | NA |
| WCM677_Z3_1 | 12 | 9356505 | 15835878 | −0.9227 | −0.994 | 1.0127 | −0.0085 | 1 | 0 |
| WCM677_Z3_1 | 12 | 16035736 | 22208202 | 0.0277 | −0.0013 | 1.031 | 0.9671 | 1 | 1 |
| WCM677_Z3_1 | 12 | 22211613 | 80714467 | 0.1311 | 0.1051 | 1.0922 | 1.059 | 1 | 1 |
| WCM677_Z3_1 | 12 | 80717554 | 133810880 | −0.007 | −0.0371 | 0.9837 | 0.9656 | 1 | 1 |
| WCM677_Z3_1 | 13 | 19600515 | 40362473 | −3.00E−04 | −0.0302 | 1.0055 | 0.953 | 1 | 1 |
| WCM677_Z3_1 | 13 | 41133678 | 70549848 | −0.9451 | −1.0177 | 1.0009 | −0.0131 | 1 | 0 |
| WCM677_Z3_1 | 13 | 70681397 | 115089411 | 0.0016 | −0.0283 | 1.0069 | 0.9543 | 1 | 1 |
| WCM677_Z3_1 | 14 | 19377710 | 107283026 | 0.0351 | 0.0063 | 1.0168 | 0.9919 | 1 | 1 |
| WCM677_Z3_1 | 15 | 20170126 | 102517641 | −0.3649 | −0.408 | 1.0016 | 0.5057 | 1 | 1 |
| WCM677_Z3_1 | 16 | 67604 | 25268368 | 0.0671 | 0.0392 | 1.0403 | 1.0149 | 1 | 1 |
| WCM677_Z3_1 | 16 | 25704145 | 32077496 | −0.8958 | −0.9654 | 1.034 | −0.0097 | 1 | 0 |
| WCM677_Z3_1 | 16 | 32486609 | 48643776 | −0.0057 | −0.0357 | 1.0057 | 0.9453 | 1 | 1 |
| WCM677_Z3_1 | 16 | 49193515 | 61935339 | −0.9206 | −0.9917 | 1.0014 | 0.0043 | 1 | 0 |
| WCM677_Z3_1 | 16 | 62055296 | 70211314 | 0.0622 | 0.0342 | 1.0599 | 0.9881 | 1 | 1 |
| WCM677_Z3_1 | 16 | 70255159 | 90233627 | −0.879 | −0.9476 | 1.0404 | −0.0035 | 1 | 0 |
| WCM677_Z3_1 | 17 | 131595 | 81083561 | 0.0488 | 0.0204 | 1.0215 | 1.0069 | 1 | 1 |
| WCM677_Z3_1 | 18 | 48238 | 78005189 | −0.0098 | −0.04 | 1.9561 | −0.0109 | 2 | 0 |
| WCM677_Z3_1 | 19 | 281483 | 59083968 | 0.0509 | 0.0225 | 1.0231 | 1.0084 | 1 | 1 |
| WCM677_Z3_1 | 2 | 41615 | 116599829 | 0.0346 | 0.0058 | 1.0164 | 0.9916 | 1 | 1 |
| WCM677_Z3_1 | 2 | 118572427 | 144728285 | −0.9389 | −1.0112 | 1.0069 | −0.0146 | 1 | 0 |
| WCM677_Z3_1 | 2 | 144764822 | 162567212 | −0.0187 | −0.0492 | 0.9755 | 0.9575 | 1 | 1 |
| WCM677_Z3_1 | 2 | 162627523 | 189348168 | −0.653 | −0.7092 | 1.001 | 0.2222 | 1 | 0 |
| WCM677_Z3_1 | 2 | 189387510 | 207655376 | 9.00E−04 | −0.029 | 0.9942 | 0.9659 | 1 | 1 |
| WCM677_Z3_1 | 2 | 207656454 | 208485406 | −0.9835 | −1.0586 | NA | NA | NA | NA |
| WCM677_Z3_1 | 2 | 208486582 | 218620790 | −2.00E−04 | −0.0302 | 1.0258 | 0.9328 | 1 | 1 |
| WCM677_Z3_1 | 2 | 218669266 | 242946607 | −0.8713 | −0.9395 | 1.0456 | −0.0028 | 1 | 0 |
| WCM677_Z3_1 | 20 | 68375 | 33080396 | −0.3343 | −0.3762 | 1.0462 | 0.4948 | 1 | 0 |
| WCM677_Z3_1 | 20 | 33092171 | 33370089 | −1.6804 | −1.8144 | NA | NA | NA | NA |
| WCM677_Z3_1 | 20 | 33433156 | 62904925 | −0.2786 | −0.3183 | 1.0811 | 0.523 | 1 | 1 |
| WCM677_Z3_1 | 21 | 9907283 | 48084204 | 0.039 | 0.0103 | 1.0217 | 0.9926 | 1 | 1 |
| WCM677_Z3_1 | 22 | 16287434 | 51215120 | −0.7392 | −0.7999 | 1.1495 | −7.00E−04 | 1 | 0 |
| WCM677_Z3_1 | 3 | 239347 | 89521717 | −0.0931 | −0.126 | 0.9988 | 0.8339 | 1 | 1 |
| WCM677_Z3_1 | 3 | 89528647 | 97634521 | 0.5268 | 0.5107 | NA | NA | NA | NA |
| WCM677_Z3_1 | 3 | 97655660 | 118878762 | −0.9462 | −1.019 | 0.9895 | −0.0026 | 1 | 0 |
| WCM677_Z3_1 | 3 | 118905658 | 197894713 | 0.4834 | 0.4664 | 1.7857 | 0.9777 | 2 | 1 |
| WCM677_Z3_1 | 4 | 59339 | 25678199 | −0.0034 | −0.0335 | 0.9841 | 0.97 | 1 | 1 |
| WCM677_Z3_1 | 4 | 25678266 | 114081521 | −0.0697 | −0.1018 | 0.9405 | 0.9232 | 1 | 1 |
| WCM677_Z3_1 | 4 | 114095568 | 117220900 | −0.1023 | −0.1356 | NA | NA | NA | NA |
| WCM677_Z3_1 | 4 | 118005531 | 135122158 | −1.0546 | −1.1344 | 0.9323 | −0.0212 | 1 | 0 |
| WCM677_Z3_1 | 4 | 138442211 | 190426502 | −0.0545 | −0.0861 | 0.9508 | 0.9333 | 1 | 1 |
| WCM677_Z3_1 | 5 | 140657 | 24593503 | −0.3577 | −0.4005 | 0.9965 | 0.5187 | 1 | 1 |
| WCM677_Z3_1 | 5 | 26881462 | 118580165 | −0.012 | −0.0423 | 0.9771 | 0.9651 | 1 | 1 |
| WCM677_Z3_1 | 5 | 118582886 | 180660174 | 0.1081 | 0.0814 | 1.069 | 1.0472 | 1 | 1 |
| WCM677_Z3_1 | 5 | 180660694 | 180687773 | 0.0045 | −0.0252 | NA | NA | NA | NA |
| WCM677_Z3_1 | 6 | 203509 | 80515697 | −0.6156 | −0.6699 | 0.9985 | 0.2586 | 1 | 0 |
| WCM677_Z3_1 | 6 | 80580038 | 96999725 | −0.9418 | −1.0142 | 0.9901 | 1.00E−04 | 1 | 0 |
| WCM677_Z3_1 | 6 | 97000464 | 170893458 | −0.6989 | −0.7574 | 0.9439 | 0.2392 | 1 | 0 |
| WCM677_Z3_1 | 7 | 193265 | 158937375 | 0.0151 | −0.0144 | 0.9962 | 0.984 | 1 | 1 |
| WCM677_Z3_1 | 8 | 190900 | 43212026 | −0.3422 | −0.3844 | 1.5392 | −0.007 | 2 | 0 |
| WCM677_Z3_1 | 8 | 47752739 | 51465632 | 1.3866 | 1.3845 | NA | NA | NA | NA |
| WCM677_Z3_1 | 8 | 51571179 | 66517770 | 0.9506 | 0.9425 | 3.8355 | 0.0081 | 4 | 0 |
| WCM677_Z3_1 | 8 | 66525522 | 146279513 | 1.4257 | 1.4241 | 3.9051 | 1.462 | 4 | 1 |
| WCM677_Z3_1 | 9 | 17091 | 8733811 | −0.0559 | −0.0876 | 0.9965 | 0.8857 | 1 | 1 |
| WCM677_Z3_1 | 9 | 12694039 | 14088265 | −0.9131 | −0.9838 | NA | NA | NA | NA |
| WCM677_Z3_1 | 9 | 14102421 | 103348634 | −0.0149 | −0.0453 | 0.9871 | 0.9511 | 1 | 1 |
| WCM677_Z3_1 | 9 | 104032333 | 112969726 | −0.7696 | −0.8319 | 1.1426 | −0.0191 | 1 | 0 |
| WCM677_Z3_1 | 9 | 113006461 | 141111414 | 0.1631 | 0.138 | 1.1128 | 1.088 | 1 | 1 |
| WCM677_Z3_1 | X | 2707734 | 154509348 | −0.0288 | −0.0596 | NA | NA | NA | NA |
| WCM677_Z6_1 | 1 | 721476 | 3809552 | 0.0476 | 0.0551 | 1.0536 | 1.0243 | 1 | 1 |
| WCM677_Z6_1 | 1 | 3816741 | 19027200 | −0.9219 | −0.9278 | 1.0507 | 6.00E−04 | 1 | 0 |
| WCM677_Z6_1 | 1 | 19029642 | 59250977 | 0.0373 | 0.0448 | 1.0378 | 1.0253 | 1 | 1 |
| WCM677_Z6_1 | 1 | 59359386 | 109655251 | −0.0714 | −0.065 | 0.9694 | 0.9424 | 1 | 1 |
| WCM677_Z6_1 | 1 | 109656269 | 145209435 | 0.0105 | 0.0177 | 1.0338 | 0.9909 | 1 | 1 |
| WCM677_Z6_1 | 1 | 145281646 | 156571231 | 0.6024 | 0.6144 | 2.0287 | 1.0332 | 2 | 1 |
| WCM677_Z6_1 | 1 | 156593281 | 160254868 | 0.0184 | 0.0257 | 1.018 | 1.018 | 1 | 1 |
| WCM677_Z6_1 | 1 | 160259944 | 249212354 | 0.5727 | 0.5845 | 1.9795 | 1.0194 | 2 | 1 |
| WCM677_Z6_1 | 10 | 225995 | 135440068 | −0.007 | 0 | 1 | 1 | 1 | 1 |
| WCM677_Z6_1 | 11 | 193097 | 103720705 | −0.0269 | −0.0202 | 0.9931 | 0.9792 | 1 | 1 |
| WCM677_Z6_1 | 11 | 103780506 | 126969289 | −0.9559 | −0.9625 | 1.026 | 4.00E−04 | 1 | 0 |
| WCM677_Z6_1 | 11 | 128332337 | 134856534 | 0.0666 | 0.0743 | 1.0869 | 1.0189 | 1 | 1 |
| WCM677_Z6_1 | 12 | 148639 | 7083589 | 0.0776 | 0.0854 | 1.0707 | 1.0514 | 1 | 1 |
| WCM677_Z6_1 | 12 | 7169192 | 7254681 | −0.8726 | −0.8776 | NA | NA | NA | NA |
| WCM677_Z6_1 | 12 | 7261027 | 7633814 | 0.0817 | 0.0896 | NA | NA | NA | NA |
| WCM677_Z6_1 | 12 | 7635293 | 8090972 | −1.0235 | −1.0315 | NA | NA | NA | NA |
| WCM677_Z6_1 | 12 | 8192440 | 8926171 | 0.2656 | 0.2751 | NA | NA | NA | NA |
| WCM677_Z6_1 | 12 | 8930211 | 9087060 | −0.9452 | −0.9516 | NA | NA | NA | NA |
| WCM677_Z6_1 | 12 | 9087756 | 9465477 | 0.3167 | 0.3266 | NA | NA | NA | NA |
| WCM677_Z6_1 | 12 | 9571646 | 15835870 | −0.9846 | −0.9918 | 1.0004 | 0.0053 | 1 | 0 |
| WCM677_Z6_1 | 12 | 16035659 | 44194268 | 0.254 | 0.2635 | 1.3916 | 1.0091 | 1 | 1 |
| WCM677_Z6_1 | 12 | 44196100 | 62954390 | 0.3685 | 0.3788 | 1.5466 | 1.0539 | 2 | 1 |
| WCM677_Z6_1 | 12 | 62954589 | 81537013 | 0.2601 | 0.2695 | 1.4376 | 0.9732 | 1 | 1 |
| WCM677_Z6_1 | 12 | 81545846 | 133810752 | −0.006 | 0.001 | 1.0048 | 0.9967 | 1 | 1 |
| WCM677_Z6_1 | 13 | 19600515 | 40362474 | −0.041 | −0.0343 | 0.9982 | 0.9548 | 1 | 1 |
| WCM677_Z6_1 | 13 | 41133678 | 70535533 | −1.0047 | −1.0123 | 0.9915 | 0 | 1 | 0 |
| WCM677_Z6_1 | 13 | 70549848 | 115089411 | −0.0074 | −4.00E−04 | 0.9997 | 0.9997 | 1 | 1 |
| WCM677_Z6_1 | 14 | 19377712 | 107283220 | 0.0051 | 0.0122 | 1.0146 | 1.0024 | 1 | 1 |
| WCM677_Z6_1 | 15 | 20169971 | 22836062 | −0.1741 | −0.1689 | 1.055 | 0.724 | 1 | 1 |
| WCM677_Z6_1 | 15 | 22840319 | 102517678 | −0.4076 | −0.4052 | 0.9992 | 0.511 | 1 | 1 |
| WCM677_Z6_1 | 16 | 67604 | 26147212 | 0.0325 | 0.0399 | 1.0312 | 1.025 | 1 | 1 |
| WCM677_Z6_1 | 16 | 26147555 | 32013471 | −0.9459 | −0.9523 | 1.0332 | 4.00E−04 | 1 | 0 |
| WCM677_Z6_1 | 16 | 32077421 | 48643763 | −0.061 | −0.0546 | 1.0115 | 0.9142 | 1 | 1 |
| WCM677_Z6_1 | 16 | 49193498 | 61935339 | −0.973 | −0.98 | 1.0137 | 2.00E−04 | 1 | 0 |
| WCM677_Z6_1 | 16 | 62055278 | 70211314 | 0.0314 | 0.0388 | 1.0407 | 1.0137 | 1 | 1 |
| WCM677_Z6_1 | 16 | 70255159 | 90233627 | −0.937 | −0.9432 | 1.0396 | 5.00E−04 | 1 | 0 |
| WCM677_Z6_1 | 17 | 131595 | 81083567 | 0.0229 | 0.0302 | 1.0211 | 1.0211 | 1 | 1 |
| WCM677_Z6_1 | 18 | 48212 | 78005226 | −0.0264 | −0.0196 | 1.9632 | 0.0098 | 2 | 0 |
| WCM677_Z6_1 | 19 | 281483 | 59083953 | 0.0268 | 0.0341 | 1.0281 | 1.0198 | 1 | 1 |
| WCM677_Z6_1 | 2 | 41620 | 116594134 | −0.0132 | −0.0062 | 0.9977 | 0.9937 | 1 | 1 |
| WCM677_Z6_1 | 2 | 116598375 | 144765061 | −0.9811 | −0.9882 | 1.008 | 2.00E−04 | 1 | 0 |
| WCM677_Z6_1 | 2 | 144899623 | 145274888 | −0.8782 | −0.8833 | NA | NA | NA | NA |
| WCM677_Z6_1 | 2 | 147345584 | 162735643 | −0.0317 | −0.0249 | 1.0007 | 0.965 | 1 | 1 |
| WCM677_Z6_1 | 2 | 162738892 | 188368391 | −0.4472 | −0.4454 | 0.9764 | 0.4924 | 1 | 0 |
| WCM677_Z6_1 | 2 | 189162253 | 218604247 | −0.0325 | −0.0258 | 0.9962 | 0.9684 | 1 | 1 |
| WCM677_Z6_1 | 2 | 218620823 | 242946616 | −0.9394 | −0.9457 | 1.0378 | 5.00E−04 | 1 | 0 |
| WCM677_Z6_1 | 20 | 68363 | 31024207 | −0.4049 | −0.4025 | 0.9996 | 0.5135 | 1 | 1 |
| WCM677_Z6_1 | 20 | 31024365 | 33080320 | −0.2518 | −0.2475 | 1.1083 | 0.5764 | 1 | 1 |
| WCM677_Z6_1 | 20 | 33092186 | 33370041 | −1.7601 | −1.7881 | NA | NA | NA | NA |
| WCM677_Z6_1 | 20 | 33433156 | 49209602 | −0.2759 | −0.2719 | 1.0823 | 0.5742 | 1 | 1 |
| WCM677_Z6_1 | 20 | 49211093 | 62904890 | −0.3783 | −0.3756 | 1.048 | 0.4936 | 1 | 0 |
| WCM677_Z6_1 | 21 | 9907315 | 48084281 | −0.0021 | 0.0049 | 1.0166 | 0.9902 | 1 | 1 |
| WCM677_Z6_1 | 22 | 16287434 | 51215153 | −0.8994 | −0.9049 | 1.0673 | 8.00E−04 | 1 | 0 |
| WCM677_Z6_1 | 3 | 239347 | 89521706 | −6.00E−04 | 0.0065 | 1.0045 | 1.0045 | 1 | 1 |
| WCM677_Z6_1 | 3 | 89528647 | 97634562 | 0.4945 | 0.5057 | NA | NA | NA | NA |
| WCM677_Z6_1 | 3 | 97655629 | 118865581 | −0.996 | −1.0035 | 0.996 | 0.0016 | 1 | 0 |
| WCM677_Z6_1 | 3 | 118865801 | 197894719 | 0.5405 | 0.5521 | 1.9386 | 0.9938 | 2 | 1 |
| WCM677_Z6_1 | 4 | 59326 | 117220889 | −0.0371 | −0.0304 | 0.987 | 0.9712 | 1 | 1 |
| WCM677_Z6_1 | 4 | 118005455 | 135122261 | −1.0232 | −1.0312 | 0.9767 | 0.0019 | 1 | 0 |
| WCM677_Z6_1 | 4 | 138442426 | 190426502 | −0.0588 | −0.0523 | 0.9771 | 0.9517 | 1 | 1 |
| WCM677_Z6_1 | 5 | 140443 | 24535387 | −0.3982 | −0.3957 | 1.0004 | 0.5198 | 1 | 1 |
| WCM677_Z6_1 | 5 | 24535914 | 50124177 | −0.0705 | −0.0641 | 0.9797 | 0.9333 | 1 | 1 |
| WCM677_Z6_1 | 5 | 50125752 | 109178078 | −0.2123 | −0.2076 | 0.9492 | 0.7828 | 1 | 1 |
| WCM677_Z6_1 | 5 | 109181596 | 109941012 | 0.0245 | 0.0319 | NA | NA | NA | NA |
| WCM677_Z6_1 | 5 | 109941963 | 110843043 | −0.3157 | −0.3122 | NA | NA | NA | NA |
| WCM677_Z6_1 | 5 | 111013692 | 154396803 | 0.1838 | 0.1926 | 1.5095 | 0.7762 | 2 | 1 |
| WCM677_Z6_1 | 5 | 154397060 | 155771579 | −1.3383 | −1.3535 | NA | NA | NA | NA |
| WCM677_Z6_1 | 5 | 155820806 | 166711862 | 0.1386 | 0.147 | 1.4782 | 0.7363 | 1 | 1 |
| WCM677_Z6_1 | 5 | 166802355 | 180687800 | 0.3229 | 0.3329 | 1.4919 | 1.0272 | 1 | 1 |
| WCM677_Z6_1 | 6 | 203545 | 80515673 | −0.4116 | −0.4093 | 0.9987 | 0.5073 | 1 | 1 |
| WCM677_Z6_1 | 6 | 80579988 | 93973575 | −1.0211 | −1.0291 | 0.9782 | 0.0019 | 1 | 0 |
| WCM677_Z6_1 | 6 | 93974399 | 170893408 | −0.4497 | −0.4479 | 0.9739 | 0.4923 | 1 | 0 |
| WCM677_Z6_1 | 7 | 193451 | 158937375 | −0.0137 | −0.0068 | 1.0044 | 0.9863 | 1 | 1 |
| WCM677_Z6_1 | 8 | 190907 | 43211989 | −0.4112 | −0.4089 | 1.5089 | −0.0025 | 2 | 0 |
| WCM677_Z6_1 | 8 | 47752745 | 51442765 | 1.3358 | 1.3516 | NA | NA | NA | NA |
| WCM677_Z6_1 | 8 | 51449236 | 66517768 | 0.9343 | 0.9483 | 3.8498 | 0.0093 | 4 | 0 |
| WCM677_Z6_1 | 8 | 66525552 | 146279472 | 1.4106 | 1.4267 | 3.8998 | 1.4767 | 4 | 1 |
| WCM677_Z6_1 | 9 | 17068 | 8733822 | −0.0617 | −0.0553 | 0.9702 | 0.9546 | 1 | 1 |
| WCM677_Z6_1 | 9 | 12694158 | 14088237 | −1.034 | −1.0422 | NA | NA | NA | NA |
| WCM677_Z6_1 | 9 | 14102457 | 103348634 | −0.0182 | −0.0113 | 1.0082 | 0.9762 | 1 | 1 |
| WCM677_Z6_1 | 9 | 104032309 | 112969854 | −0.4994 | −0.4983 | 1.4102 | 0.0057 | 1 | 0 |
| WCM677_Z6_1 | 9 | 113006491 | 141111414 | 0.3648 | 0.3751 | 1.5401 | 1.0538 | 2 | 1 |
| WCM677_Z6_1 | X | 2707708 | 154509348 | −0.0588 | −0.0523 | NA | NA | NA | NA |
| WCM677_Z7_1 | 1 | 721415 | 3816741 | 0.0389 | 0.0605 | 1.0513 | 1.0344 | 1 | 1 |
| WCM677_Z7_1 | 1 | 4715508 | 18962797 | −0.9259 | −0.9177 | 1.0527 | 0.0061 | 1 | 0 |
| WCM677_Z7_1 | 1 | 19018405 | 60299887 | 0.0165 | 0.0379 | 1.0349 | 1.0183 | 1 | 1 |
| WCM677_Z7_1 | 1 | 60301047 | 109486557 | −0.1033 | −0.0831 | 0.9516 | 0.9364 | 1 | 1 |
| WCM677_Z7_1 | 1 | 109493049 | 145209396 | −0.0159 | 0.0052 | 1.0827 | 0.9245 | 1 | 1 |
| WCM677_Z7_1 | 1 | 145281653 | 156571231 | 0.5828 | 0.6088 | 2.0039 | 1.046 | 2 | 1 |
| WCM677_Z7_1 | 1 | 156593337 | 160260395 | 0.0068 | 0.0281 | 1.0372 | 1.0022 | 1 | 1 |
| WCM677_Z7_1 | 1 | 160261169 | 249212342 | 0.5421 | 0.5678 | 1.9732 | 0.9913 | 2 | 1 |
| WCM677_Z7_1 | 10 | 225960 | 135440117 | −0.0237 | −0.0027 | 0.9981 | 0.9981 | 1 | 1 |
| WCM677_Z7_1 | 11 | 193112 | 103720705 | −0.0513 | −0.0306 | 0.9889 | 0.9691 | 1 | 1 |
| WCM677_Z7_1 | 11 | 103780446 | 126969303 | −0.9667 | −0.9593 | 1.023 | 0.0056 | 1 | 0 |
| WCM677_Z7_1 | 11 | 128332453 | 134856626 | 0.0213 | 0.0427 | 1.0987 | 0.9614 | 1 | 1 |
| WCM677_Z7_1 | 12 | 148673 | 7083589 | 0.0282 | 0.0497 | 1.0497 | 1.0204 | 1 | 1 |
| WCM677_Z7_1 | 12 | 7169149 | 7254681 | −0.853 | −0.8434 | NA | NA | NA | NA |
| WCM677_Z7_1 | 12 | 7261012 | 7633833 | −0.0231 | −0.0021 | NA | NA | NA | NA |
| WCM677_Z7_1 | 12 | 7635291 | 8192527 | −1.0501 | −1.0443 | NA | NA | NA | NA |
| WCM677_Z7_1 | 12 | 8192718 | 8925979 | 0.3169 | 0.3409 | NA | NA | NA | NA |
| WCM677_Z7_1 | 12 | 8930225 | 9087059 | −0.8465 | −0.8368 | NA | NA | NA | NA |
| WCM677_Z7_1 | 12 | 9087727 | 9465477 | 0.3275 | 0.3517 | NA | NA | NA | NA |
| WCM677_Z7_1 | 12 | 9571416 | 15835878 | −1.0046 | −0.9979 | 1.0013 | 1.00E−04 | 1 | 0 |
| WCM677_Z7_1 | 12 | 16035656 | 48073547 | 0.2358 | 0.2592 | 1.4623 | 0.9313 | 1 | 1 |
| WCM677_Z7_1 | 12 | 48075570 | 62946748 | 0.345 | 0.3693 | 1.5235 | 1.06 | 2 | 1 |
| WCM677_Z7_1 | 12 | 62949801 | 81102650 | 0.2452 | 0.2687 | 1.4489 | 0.9605 | 1 | 1 |
| WCM677_Z7_1 | 12 | 81110946 | 133810855 | −0.0092 | 0.0119 | 1.0113 | 1.0052 | 1 | 1 |
| WCM677_Z7_1 | 13 | 19600515 | 40362489 | −0.0708 | −0.0503 | 0.9872 | 0.9443 | 1 | 1 |
| WCM677_Z7_1 | 13 | 41133803 | 70535457 | −1.0136 | −1.0071 | 0.993 | 0.0021 | 1 | 0 |
| WCM677_Z7_1 | 13 | 70549886 | 115089411 | −0.0535 | −0.0328 | 0.9982 | 0.9568 | 1 | 1 |
| WCM677_Z7_1 | 14 | 19377718 | 107283019 | −0.0083 | 0.0128 | 1.0089 | 1.0089 | 1 | 1 |
| WCM677_Z7_1 | 15 | 20170045 | 102517639 | −0.4209 | −0.4046 | 1.0058 | 0.5051 | 1 | 1 |
| WCM677_Z7_1 | 16 | 67604 | 26147480 | 0.0243 | 0.0458 | 1.0395 | 1.0249 | 1 | 1 |
| WCM677_Z7_1 | 16 | 27078366 | 32013455 | −0.9525 | −0.9448 | 1.0327 | 0.0063 | 1 | 0 |
| WCM677_Z7_1 | 16 | 32077430 | 48643740 | −0.0621 | −0.0415 | 1.0158 | 0.9275 | 1 | 1 |
| WCM677_Z7_1 | 16 | 49193569 | 61935266 | −0.9383 | −0.9303 | 1.0489 | 6.00E−04 | 1 | 0 |
| WCM677_Z7_1 | 16 | 61935359 | 70211272 | 0.0337 | 0.0553 | 1.0391 | 1.0391 | 1 | 1 |
| WCM677_Z7_1 | 16 | 70255159 | 90233626 | −0.9354 | −0.9273 | 1.0457 | 0.006 | 1 | 0 |
| WCM677_Z7_1 | 17 | 131595 | 81083567 | 0.019 | 0.0404 | 1.0284 | 1.0284 | 1 | 1 |
| WCM677_Z7_1 | 18 | 48221 | 78005226 | −0.0445 | −0.0238 | 1.9675 | −2.00E−04 | 2 | 0 |
| WCM677_Z7_1 | 19 | 281498 | 59083923 | 0.0227 | 0.0441 | 1.0352 | 1.0269 | 1 | 1 |
| WCM677_Z7_1 | 2 | 41614 | 116599858 | −0.0328 | −0.0119 | 1.0018 | 0.9817 | 1 | 1 |
| WCM677_Z7_1 | 2 | 118572428 | 144765055 | −0.9926 | −0.9857 | 1.0041 | 0.0059 | 1 | 0 |
| WCM677_Z7_1 | 2 | 144899591 | 147345860 | −0.8787 | −0.8696 | NA | NA | NA | NA |
| WCM677_Z7_1 | 2 | 147346233 | 162567212 | −0.0518 | −0.0311 | 1.0044 | 0.953 | 1 | 1 |
| WCM677_Z7_1 | 2 | 162627511 | 189393878 | −0.4759 | −0.4603 | 0.9686 | 0.4851 | 1 | 0 |
| WCM677_Z7_1 | 2 | 189405938 | 218604233 | −0.0679 | −0.0474 | 0.9804 | 0.955 | 1 | 1 |
| WCM677_Z7_1 | 2 | 218620799 | 242946607 | −0.956 | −0.9483 | 1.036 | 5.00E−04 | 1 | 0 |
| WCM677_Z7_1 | 20 | 68406 | 33077679 | −0.3884 | −0.3716 | 1.0313 | 0.5145 | 1 | 1 |
| WCM677_Z7_1 | 20 | 33080320 | 33433235 | −1.7355 | −1.7481 | NA | NA | NA | NA |
| WCM677_Z7_1 | 20 | 33437999 | 62904835 | −0.3336 | −0.3161 | 1.0562 | 0.5503 | 1 | 1 |
| WCM677_Z7_1 | 21 | 9907301 | 48084210 | −0.0069 | 0.0142 | 1.0181 | 1.0018 | 1 | 1 |
| WCM677_Z7_1 | 22 | 16287443 | 51215123 | −0.6417 | −0.6286 | 1.2906 | 0.003 | 1 | 0 |
| WCM677_Z7_1 | 3 | 239347 | 89499501 | −0.0101 | 0.011 | 1.0077 | 1.0077 | 1 | 1 |
| WCM677_Z7_1 | 3 | 89521648 | 97634536 | 0.4572 | 0.4823 | NA | NA | NA | NA |
| WCM677_Z7_1 | 3 | 97655739 | 118878762 | −1.045 | −1.0391 | 0.9704 | 0.0028 | 1 | 0 |
| WCM677_Z7_1 | 3 | 118905658 | 197894713 | 0.5206 | 0.5462 | 1.941 | 0.9794 | 2 | 1 |
| WCM677_Z7_1 | 4 | 59434 | 117220900 | −0.0528 | −0.0321 | 0.978 | 0.978 | 1 | 1 |
| WCM677_Z7_1 | 4 | 118005573 | 135122340 | −1.0805 | −1.0754 | 0.9495 | −4.00E−04 | 1 | 0 |
| WCM677_Z7_1 | 4 | 138442422 | 190426502 | −0.0739 | −0.0534 | 0.9724 | 0.9549 | 1 | 1 |
| WCM677_Z7_1 | 5 | 140683 | 24537589 | −0.4312 | −0.415 | 0.9993 | 0.5007 | 1 | 1 |
| WCM677_Z7_1 | 5 | 24593567 | 111496834 | −0.0769 | −0.0565 | 0.9771 | 0.946 | 1 | 1 |
| WCM677_Z7_1 | 5 | 111497290 | 180687803 | 0.2939 | 0.3177 | 1.5065 | 0.9863 | 2 | 1 |
| WCM677_Z7_1 | 6 | 203515 | 80201426 | −0.4281 | −0.4118 | 0.9817 | 0.5216 | 1 | 1 |
| WCM677_Z7_1 | 6 | 80203460 | 97001314 | −0.9741 | −0.9668 | 1.0198 | 0.0035 | 1 | 0 |
| WCM677_Z7_1 | 6 | 97051521 | 170893406 | −0.4795 | −0.464 | 0.9676 | 0.4824 | 1 | 0 |
| WCM677_Z7_1 | 7 | 193265 | 158937416 | −0.0272 | −0.0063 | 1.0027 | 0.9886 | 1 | 1 |
| WCM677_Z7_1 | 8 | 190908 | 43211998 | −0.428 | −0.4117 | 1.4983 | 0.0051 | 1 | 0 |
| WCM677_Z7_1 | 8 | 47752730 | 51465616 | 1.2928 | 1.3225 | NA | NA | NA | NA |
| WCM677_Z7_1 | 8 | 51571151 | 66517769 | 0.9117 | 0.9397 | 3.827 | 0.0092 | 4 | 0 |
| WCM677_Z7_1 | 8 | 66525560 | 146279529 | 1.3912 | 1.4213 | 3.9041 | 1.4523 | 4 | 1 |
| WCM677_Z7_1 | 9 | 17091 | 8733816 | −0.0472 | −0.0265 | 1.0076 | 0.956 | 1 | 1 |
| WCM677_Z7_1 | 9 | 12694081 | 14088237 | −0.9355 | −0.9274 | NA | NA | NA | NA |
| WCM677_Z7_1 | 9 | 14102457 | 103348634 | −0.0288 | −0.0079 | 1.0026 | 0.9865 | 1 | 1 |
| WCM677_Z7_1 | 9 | 104032333 | 113013157 | −0.5335 | −0.5188 | 1.3905 | 0.0055 | 1 | 0 |
| WCM677_Z7_1 | 9 | 113013683 | 141111414 | 0.3409 | 0.3651 | 1.5294 | 1.0465 | 2 | 1 |
| WCM677_Z7_1 | X | 2707724 | 154509348 | −0.0679 | −0.0473 | NA | NA | NA | NA |
| WCM677_Z9_1 | 1 | 721439 | 3809475 | 0.0581 | 0.0658 | 1.0635 | 1.0297 | 1 | 1 |
| WCM677_Z9_1 | 1 | 3816750 | 18962816 | −0.9563 | −0.9629 | 1.0257 | 4.00E−04 | 1 | 0 |
| WCM677_Z9_1 | 1 | 19018405 | 55551640 | 0.0149 | 0.0221 | 1.0155 | 1.0155 | 1 | 1 |
| WCM677_Z9_1 | 1 | 55555353 | 109159012 | −0.0857 | −0.0795 | 0.9627 | 0.9301 | 1 | 1 |
| WCM677_Z9_1 | 1 | 109167290 | 145115810 | −0.0038 | 0.0033 | 1.1055 | 0.899 | 1 | 1 |
| WCM677_Z9_1 | 1 | 145209432 | 156568212 | 0.5853 | 0.5972 | 2.0153 | 1.0102 | 2 | 1 |
| WCM677_Z9_1 | 1 | 156568774 | 160254868 | 0.0149 | 0.0221 | 1.0319 | 0.999 | 1 | 1 |
| WCM677_Z9_1 | 1 | 160259957 | 249212373 | 0.5623 | 0.5741 | 1.9803 | 0.9971 | 2 | 1 |
| WCM677_Z9_1 | 10 | 225995 | 135440068 | −0.0113 | −0.0043 | 0.997 | 0.997 | 1 | 1 |
| WCM677_Z9_1 | 11 | 193124 | 103327014 | 0.0014 | 0.0085 | 1.0099 | 1.0018 | 1 | 1 |
| WCM677_Z9_1 | 11 | 103339409 | 126969232 | −0.956 | −0.9626 | 1.0259 | 4.00E−04 | 1 | 0 |
| WCM677_Z9_1 | 11 | 128332352 | 134856626 | 0.0132 | 0.0204 | 1.0409 | 0.9876 | 1 | 1 |
| WCM677_Z9_1 | 12 | 148674 | 7632562 | 0.0389 | 0.0463 | 1.0504 | 1.0149 | 1 | 1 |
| WCM677_Z9_1 | 12 | 7633843 | 8090972 | −1.0598 | −1.0686 | NA | NA | NA | NA |
| WCM677_Z9_1 | 12 | 8192459 | 8926041 | 0.2845 | 0.2941 | NA | NA | NA | NA |
| WCM677_Z9_1 | 12 | 8930225 | 9087073 | −0.9295 | −0.9356 | NA | NA | NA | NA |
| WCM677_Z9_1 | 12 | 9087727 | 9465276 | 0.3365 | 0.3466 | NA | NA | NA | NA |
| WCM677_Z9_1 | 12 | 9571646 | 15835866 | −0.959 | −0.9656 | 1.0227 | 0.0014 | 1 | 0 |
| WCM677_Z9_1 | 12 | 16035648 | 48073545 | 0.2598 | 0.2693 | 1.4859 | 0.9245 | 1 | 1 |
| WCM677_Z9_1 | 12 | 48075570 | 62946748 | 0.3628 | 0.3731 | 1.5026 | 1.0876 | 2 | 1 |
| WCM677_Z9_1 | 12 | 62949801 | 81101832 | 0.2671 | 0.2766 | 1.4422 | 0.9805 | 1 | 1 |
| WCM677_Z9_1 | 12 | 81102375 | 133810842 | −0.0172 | −0.0103 | 0.9929 | 0.9929 | 1 | 1 |
| WCM677_Z9_1 | 13 | 19600515 | 40362474 | −0.0474 | −0.0409 | 0.9799 | 0.9642 | 1 | 1 |
| WCM677_Z9_1 | 13 | 41133892 | 70535555 | −1.0087 | −1.0164 | 0.9887 | 0 | 1 | 0 |
| WCM677_Z9_1 | 13 | 70549887 | 115089411 | −0.023 | −0.0162 | 0.9928 | 0.9848 | 1 | 1 |
| WCM677_Z9_1 | 14 | 19377726 | 107283213 | 0.0027 | 0.0098 | 1.0068 | 1.0068 | 1 | 1 |
| WCM677_Z9_1 | 15 | 20170006 | 102517642 | −0.4198 | −0.4176 | 0.9937 | 0.5036 | 1 | 1 |
| WCM677_Z9_1 | 16 | 67604 | 27078374 | 0.0252 | 0.0325 | 1.03 | 1.0156 | 1 | 1 |
| WCM677_Z9_1 | 16 | 27078548 | 32013471 | −0.9702 | −0.9771 | 1.0158 | 3.00E−04 | 1 | 0 |
| WCM677_Z9_1 | 16 | 32077478 | 48643776 | −0.0645 | −0.058 | 0.9877 | 0.9334 | 1 | 1 |
| WCM677_Z9_1 | 16 | 49193569 | 61935111 | −0.9608 | −0.9675 | 1.0224 | 3.00E−04 | 1 | 0 |
| WCM677_Z9_1 | 16 | 61935339 | 70211290 | 0.0416 | 0.049 | 1.0513 | 1.0179 | 1 | 1 |
| WCM677_Z9_1 | 16 | 70255159 | 90233627 | −0.9529 | −0.9594 | 1.0281 | 4.00E−04 | 1 | 0 |
| WCM677_Z9_1 | 17 | 131595 | 81083561 | 0.0146 | 0.0218 | 1.0152 | 1.0152 | 1 | 1 |
| WCM677_Z9_1 | 18 | 48203 | 78005199 | −0.0266 | −0.0198 | 1.9729 | −1.00E−04 | 2 | 0 |
| WCM677_Z9_1 | 19 | 281415 | 59083985 | 0.0041 | 0.0112 | 1.0118 | 1.0037 | 1 | 1 |
| WCM677_Z9_1 | 2 | 41612 | 116573267 | −0.0156 | −0.0087 | 1.001 | 0.987 | 1 | 1 |
| WCM677_Z9_1 | 2 | 116593754 | 145271267 | −0.9766 | −0.9836 | 1.0112 | 2.00E−04 | 1 | 0 |
| WCM677_Z9_1 | 2 | 145274869 | 162567203 | −0.0351 | −0.0284 | 1.0043 | 0.9568 | 1 | 1 |
| WCM677_Z9_1 | 2 | 162627550 | 188211112 | −0.4407 | −0.4388 | 0.9898 | 0.4857 | 1 | 0 |
| WCM677_Z9_1 | 2 | 188216855 | 207656422 | −0.0345 | −0.0278 | 1.0166 | 0.9453 | 1 | 1 |
| WCM677_Z9_1 | 2 | 207814367 | 208485372 | −1.0114 | −1.0191 | NA | NA | NA | NA |
| WCM677_Z9_1 | 2 | 208486562 | 218604289 | −0.0296 | −0.0228 | 1.0211 | 0.9475 | 1 | 1 |
| WCM677_Z9_1 | 2 | 218620790 | 242946616 | −0.9368 | −0.943 | 1.0398 | 5.00E−04 | 1 | 0 |
| WCM677_Z9_1 | 20 | 68351 | 31383283 | −0.4083 | −0.406 | 1.0086 | 0.5008 | 1 | 1 |
| WCM677_Z9_1 | 20 | 31383484 | 33077692 | −0.2456 | −0.2412 | NA | NA | NA | NA |
| WCM677_Z9_1 | 20 | 33080320 | 33370121 | −1.7932 | −1.8224 | NA | NA | NA | NA |
| WCM677_Z9_1 | 20 | 33433294 | 49621339 | −0.2992 | −0.2954 | 1.0714 | 0.5583 | 1 | 1 |
| WCM677_Z9_1 | 20 | 49626314 | 62904863 | −0.3824 | −0.3798 | 1.0247 | 0.5124 | 1 | 1 |
| WCM677_Z9_1 | 21 | 9907318 | 48084282 | −0.0056 | 0.0014 | 1.0101 | 0.9919 | 1 | 1 |
| WCM677_Z9_1 | 22 | 16287439 | 51215115 | −0.5432 | −0.5428 | 1.3691 | 0.0038 | 1 | 0 |
| WCM677_Z9_1 | 3 | 239347 | 97634561 | −2.00E−04 | 0.0068 | 1.0088 | 1.0007 | 1 | 1 |
| WCM677_Z9_1 | 3 | 97655720 | 118865604 | −0.991 | −0.9983 | 1.0011 | 1.00E−04 | 1 | 0 |
| WCM677_Z9_1 | 3 | 118865801 | 197894726 | 0.5416 | 0.5532 | 1.9549 | 0.9798 | 2 | 1 |
| WCM677_Z9_1 | 4 | 59326 | 117220908 | −0.0334 | −0.0267 | 0.9817 | 0.9817 | 1 | 1 |
| WCM677_Z9_1 | 4 | 118005614 | 135122242 | −1.0209 | −1.0289 | 0.9803 | −1.00E−04 | 1 | 0 |
| WCM677_Z9_1 | 4 | 138442429 | 190426502 | −0.0625 | −0.0561 | 0.9697 | 0.9541 | 1 | 1 |
| WCM677_Z9_1 | 5 | 140436 | 24593567 | −0.4014 | −0.399 | 0.9759 | 0.5409 | 1 | 1 |
| WCM677_Z9_1 | 5 | 26881275 | 110843047 | −0.0569 | −0.0505 | 0.9783 | 0.9529 | 1 | 1 |
| WCM677_Z9_1 | 5 | 111013695 | 180687776 | 0.312 | 0.3219 | 1.4969 | 1.003 | 1 | 1 |
| WCM677_Z9_1 | 6 | 203542 | 80222917 | −0.4018 | −0.3994 | 1.0101 | 0.5062 | 1 | 1 |
| WCM677_Z9_1 | 6 | 80223223 | 92231400 | −0.992 | −0.9994 | 1.0003 | 1.00E−04 | 1 | 0 |
| WCM677_Z9_1 | 6 | 93953172 | 170893438 | −0.46 | −0.4584 | 0.9713 | 0.4843 | 1 | 0 |
| WCM677_Z9_1 | 7 | 193265 | 158937416 | −0.0045 | 0.0025 | 1.0078 | 0.9957 | 1 | 1 |
| WCM677_Z9_1 | 8 | 190908 | 43212019 | −0.4114 | −0.4091 | 1.5086 | −0.0025 | 2 | 0 |
| WCM677_Z9_1 | 8 | 47752745 | 51465617 | 1.3881 | 1.4041 | NA | NA | NA | NA |
| WCM677_Z9_1 | 8 | 51571149 | 65509244 | 0.9353 | 0.9492 | 3.8524 | 0.0093 | 4 | 0 |
| WCM677_Z9_1 | 8 | 65517340 | 146279489 | 1.411 | 1.427 | 3.917 | 1.4609 | 4 | 1 |
| WCM677_Z9_1 | 9 | 17116 | 8733795 | −0.0505 | −0.044 | 1.0278 | 0.9121 | 1 | 1 |
| WCM677_Z9_1 | 9 | 12694236 | 14088271 | −1.0484 | −1.0569 | NA | NA | NA | NA |
| WCM677_Z9_1 | 9 | 14102457 | 103348634 | −0.0107 | −0.0037 | 1.0126 | 0.9823 | 1 | 1 |
| WCM677_Z9_1 | 9 | 104032203 | 112969822 | −0.4759 | −0.4746 | 1.4349 | 0.0045 | 1 | 0 |
| WCM677_Z9_1 | 9 | 113006491 | 141111414 | 0.3449 | 0.355 | 1.5188 | 1.0392 | 2 | 1 |
| WCM677_Z9_1 | X | 2707711 | 154509348 | −0.0567 | −0.0502 | NA | NA | NA | NA |
| WCM6_Z5_1 | 1 | 809582 | 51180660 | −0.2943 | 0.6039 | 2.014 | 1.0257 | 2 | 1 |
| WCM6_Z5_1 | 1 | 51210433 | 62299351 | 0.0701 | 0.9916 | 2.9941 | 0.9827 | 3 | 1 |
| WCM6_Z5_1 | 1 | 62321782 | 72748079 | 0.3301 | 1.2649 | 2.8431 | 1.9631 | 3 | 2 |
| WCM6_Z5_1 | 1 | 74492587 | 104163135 | −0.8472 | 9.00E−04 | 1.9634 | 0.0379 | 2 | 0 |
| WCM6_Z5_1 | 1 | 107599703 | 249212341 | 0.3592 | 1.2953 | 2.9469 | 1.9618 | 3 | 2 |
| WCM6_Z5_1 | 10 | 255895 | 8115989 | −0.8853 | −0.0415 | 1.8562 | 0.087 | 2 | 0 |
| WCM6_Z5_1 | 10 | 11060033 | 18331789 | 1.0374 | 1.9978 | 6.2188 | 1.769 | 6 | 2 |
| WCM6_Z5_1 | 10 | 18430123 | 18803448 | 0.6107 | 1.5573 | NA | NA | NA | NA |
| WCM6_Z5_1 | 10 | 18803989 | 18962937 | 0.3624 | 1.2987 | NA | NA | NA | NA |
| WCM6_Z5_1 | 10 | 18964170 | 21435454 | −0.7912 | 0.063 | NA | NA | NA | NA |
| WCM6_Z5_1 | 10 | 21461338 | 22856835 | 0.3448 | 1.2803 | NA | NA | NA | NA |
| WCM6_Z5_1 | 10 | 22862202 | 28908551 | 0.636 | 1.5835 | 4.0101 | 1.9837 | 4 | 2 |
| WCM6_Z5_1 | 10 | 28966936 | 38650270 | −0.8943 | −0.0516 | 1.8956 | 0.0341 | 2 | 0 |
| WCM6_Z5_1 | 10 | 38654529 | 46962074 | 0.3743 | 1.3111 | 2.8027 | 2.1598 | 3 | 2 |
| WCM6_Z5_1 | 10 | 46963813 | 49687666 | −0.7215 | 0.1399 | NA | NA | NA | NA |
| WCM6_Z5_1 | 10 | 49791169 | 53059393 | 0.2041 | 1.1328 | NA | NA | NA | NA |
| WCM6_Z5_1 | 10 | 53227568 | 61122203 | −0.5523 | 0.325 | NA | NA | NA | NA |
| WCM6_Z5_1 | 10 | 61412614 | 64575610 | 0.1842 | 1.1119 | NA | NA | NA | NA |
| WCM6_Z5_1 | 10 | 64893248 | 69106445 | −0.7002 | 0.1633 | NA | NA | NA | NA |
| WCM6_Z5_1 | 10 | 69281686 | 70405593 | 0.5914 | 1.5372 | 3.8243 | 1.9802 | 4 | 2 |
| WCM6_Z5_1 | 10 | 70406062 | 71921623 | 0.0653 | 0.9866 | 2.1196 | 1.8434 | 2 | 2 |
| WCM6_Z5_1 | 10 | 71964679 | 75277538 | 0.6375 | 1.585 | 3.8931 | 2.1072 | 4 | 2 |
| WCM6_Z5_1 | 10 | 75279549 | 82369257 | 1.2772 | 2.2438 | 7.4413 | 2.0315 | 7 | 2 |
| WCM6_Z5_1 | 10 | 83635291 | 88260290 | 0.0998 | 1.0231 | NA | NA | NA | NA |
| WCM6_Z5_1 | 10 | 88277518 | 135440062 | −0.8439 | 0.0045 | 1.9481 | 0.0583 | 2 | 0 |
| WCM6_Z5_1 | 11 | 193112 | 65268919 | −0.3324 | 0.563 | 1.9586 | 0.996 | 2 | 1 |
| WCM6_Z5_1 | 11 | 65269375 | 78467874 | 0.0627 | 0.9839 | 1.9966 | 1.959 | 2 | 2 |
| WCM6_Z5_1 | 11 | 78481998 | 134257515 | −0.3208 | 0.5754 | 1.9849 | 0.9953 | 2 | 1 |
| WCM6_Z5_1 | 12 | 283766 | 78583891 | 0.0379 | 0.9576 | 1.9544 | 1.9298 | 2 | 2 |
| WCM6_Z5_1 | 12 | 78591073 | 81839440 | −0.3757 | 0.5164 | 2.0408 | 0.82 | 2 | 1 |
| WCM6_Z5_1 | 12 | 81851545 | 114793866 | 0.0339 | 0.9534 | 1.9712 | 1.9016 | 2 | 2 |
| WCM6_Z5_1 | 12 | 114804034 | 115120914 | −0.9246 | −0.0853 | NA | NA | NA | NA |
| WCM6_Z5_1 | 12 | 116399206 | 133779169 | 0.0559 | 0.9766 | 1.9928 | 1.9429 | 2 | 2 |
| WCM6_Z5_1 | 13 | 19747994 | 24243145 | 1.07 | 2.0314 | 7.3547 | 0.8211 | 7 | 1 |
| WCM6_Z5_1 | 13 | 24304550 | 41949696 | −0.3365 | 0.5586 | 1.9353 | 1.0103 | 2 | 1 |
| WCM6_Z5_1 | 13 | 42032482 | 75860900 | −1.5814 | −0.8437 | 1.0411 | 0.0733 | 1 | 0 |
| WCM6_Z5_1 | 13 | 75863089 | 115089411 | −0.3248 | 0.5711 | 1.944 | 1.0274 | 2 | 1 |
| WCM6_Z5_1 | 14 | 19377685 | 107283026 | 0.0404 | 0.9602 | 1.96 | 1.9312 | 2 | 2 |
| WCM6_Z5_1 | 15 | 20170045 | 40510711 | −0.7084 | 0.1544 | 1.2978 | 0.9281 | 1 | 1 |
| WCM6_Z5_1 | 15 | 40512943 | 40920783 | −1.5851 | −0.8482 | NA | NA | NA | NA |
| WCM6_Z5_1 | 15 | 40921518 | 43426191 | −0.6971 | 0.1667 | 1.3815 | 0.8634 | 1 | 1 |
| WCM6_Z5_1 | 15 | 43426454 | 43940187 | −1.4988 | −0.7453 | 1.0891 | 0.104 | 1 | 0 |
| WCM6_Z5_1 | 15 | 43988604 | 102514154 | −0.7073 | 0.1555 | 1.2583 | 0.9693 | 1 | 1 |
| WCM6_Z5_1 | 16 | 79914 | 11370138 | 0.0429 | 0.9629 | 1.974 | 1.9246 | 2 | 2 |
| WCM6_Z5_1 | 16 | 11374866 | 12142278 | −0.8772 | −0.0325 | NA | NA | NA | NA |
| WCM6_Z5_1 | 16 | 12145860 | 18903605 | −8.00E−04 | 0.9167 | 1.9237 | 1.8518 | 2 | 2 |
| WCM6_Z5_1 | 16 | 19020458 | 19562544 | −0.8397 | 0.0092 | NA | NA | NA | NA |
| WCM6_Z5_1 | 16 | 19576164 | 20781523 | −3.2602 | −3.3564 | NA | NA | NA | NA |
| WCM6_Z5_1 | 16 | 20787375 | 21128601 | −0.7569 | 0.1009 | NA | NA | NA | NA |
| WCM6_Z5_1 | 16 | 21132099 | 57007445 | 0.057 | 0.9778 | 1.9861 | 1.9528 | 2 | 2 |
| WCM6_Z5_1 | 16 | 57012086 | 90161530 | −0.8239 | 0.0268 | 1.9983 | 0.0392 | 2 | 0 |
| WCM6_Z5_1 | 17 | 131595 | 19186977 | −0.3352 | 0.56 | 1.9325 | 1.0161 | 2 | 1 |
| WCM6_Z5_1 | 17 | 19188994 | 81052298 | 0.3371 | 1.2722 | 2.8802 | 1.9504 | 3 | 2 |
| WCM6_Z5_1 | 18 | 47669 | 57026471 | −0.8323 | 0.0174 | 1.1031 | 0.9212 | 1 | 1 |
| WCM6_Z5_1 | 18 | 57103214 | 78005224 | −1.5838 | −0.8466 | 1.0712 | 0.041 | 1 | 0 |
| WCM6_Z5_1 | 19 | 281534 | 1506220 | 0.0084 | 0.9264 | 1.9408 | 1.8603 | 2 | 2 |
| WCM6_Z5_1 | 19 | 1506286 | 3028766 | −0.7736 | 0.0824 | 2.0696 | 0.048 | 2 | 0 |
| WCM6_Z5_1 | 19 | 3053838 | 3118983 | −3.0698 | −2.9762 | NA | NA | NA | NA |
| WCM6_Z5_1 | 19 | 3136470 | 4366925 | −0.7922 | 0.0619 | 2.0786 | 0.009 | 2 | 0 |
| WCM6_Z5_1 | 19 | 4402786 | 17750672 | 0.058 | 0.9789 | 1.9876 | 1.9543 | 2 | 2 |
| WCM6_Z5_1 | 19 | 17751398 | 17932138 | −0.8689 | −0.0232 | NA | NA | NA | NA |
| WCM6_Z5_1 | 19 | 17937588 | 42636501 | 0.0381 | 0.9579 | 1.9733 | 1.9116 | 2 | 2 |
| WCM6_Z5_1 | 19 | 42703764 | 48018114 | −0.8439 | 0.0045 | 1.9781 | 0.0282 | 2 | 0 |
| WCM6_Z5_1 | 19 | 48176904 | 59082615 | 0.0403 | 0.9601 | 1.9661 | 1.9249 | 2 | 2 |
| WCM6_Z5_1 | 2 | 41602 | 33811712 | −0.3057 | 0.5917 | 1.9536 | 1.0604 | 2 | 1 |
| WCM6_Z5_1 | 2 | 33813495 | 80846314 | −0.8254 | 0.0251 | 1.1677 | 0.8674 | 1 | 1 |
| WCM6_Z5_1 | 2 | 80874686 | 125504906 | −0.3278 | 0.5679 | 1.962 | 1.0027 | 2 | 1 |
| WCM6_Z5_1 | 2 | 125521391 | 144709545 | −0.8101 | 0.0421 | 1.138 | 0.9212 | 1 | 1 |
| WCM6_Z5_1 | 2 | 144710346 | 242815257 | −0.3123 | 0.5846 | 1.9941 | 1.0051 | 2 | 1 |
| WCM6_Z5_1 | 20 | 68413 | 947801 | 0.058 | 0.9789 | 2.0147 | 1.9272 | 2 | 2 |
| WCM6_Z5_1 | 20 | 948665 | 23967268 | −0.2462 | 0.6554 | 2.1552 | 0.995 | 2 | 1 |
| WCM6_Z5_1 | 20 | 24523781 | 62904982 | 0.1061 | 1.0296 | 2.0673 | 2.0157 | 2 | 2 |
| WCM6_Z5_1 | 21 | 10862853 | 48084281 | 0.0299 | 0.9492 | 1.9512 | 1.9104 | 2 | 2 |
| WCM6_Z5_1 | 22 | 16287704 | 51215123 | −0.7755 | 0.0803 | 2.0725 | 0.0419 | 2 | 0 |
| WCM6_Z5_1 | 3 | 361501 | 96533521 | 0.6032 | 1.5495 | 3.9175 | 1.9366 | 4 | 2 |
| WCM6_Z5_1 | 3 | 96533819 | 98311874 | 0.9653 | 1.9237 | 4.1219 | 3.4658 | 4 | 3 |
| WCM6_Z5_1 | 3 | 98312138 | 140140107 | 0.5919 | 1.5377 | 3.889 | 1.9176 | 4 | 2 |
| WCM6_Z5_1 | 3 | 140167508 | 140284912 | −0.6298 | 0.2406 | NA | NA | NA | NA |
| WCM6_Z5_1 | 3 | 140397256 | 197847250 | 0.5823 | 1.5277 | 3.8715 | 1.8952 | 4 | 2 |
| WCM6_Z5_1 | 4 | 53361 | 166418697 | 0.0421 | 0.9621 | 1.9625 | 1.9337 | 2 | 2 |
| WCM6_Z5_1 | 4 | 166795070 | 169602489 | 0.5216 | 1.4647 | NA | NA | NA | NA |
| WCM6_Z5_1 | 4 | 169604196 | 189068390 | 0.0611 | 0.9822 | 2.0151 | 1.9358 | 2 | 2 |
| WCM6_Z5_1 | 5 | 140568 | 83550040 | −0.3426 | 0.552 | 1.9866 | 0.9456 | 2 | 1 |
| WCM6_Z5_1 | 5 | 83680098 | 98195745 | −0.8613 | −0.0148 | 1.9684 | 0.0111 | 2 | 0 |
| WCM6_Z5_1 | 5 | 98209377 | 108168579 | −2.9332 | −2.7299 | NA | NA | NA | NA |
| WCM6_Z5_1 | 5 | 108203574 | 122012328 | −0.8485 | −6.00E−04 | 1.9526 | 0.0466 | 2 | 0 |
| WCM6_Z5_1 | 5 | 122110904 | 151230962 | −0.3179 | 0.5785 | 1.9506 | 1.036 | 2 | 1 |
| WCM6_Z5_1 | 5 | 151234644 | 161580404 | −0.816 | 0.0355 | 1.9565 | 0.0934 | 2 | 0 |
| WCM6_Z5_1 | 5 | 162866423 | 169812455 | −0.3955 | 0.4951 | 2.0951 | 0.7236 | 2 | 1 |
| WCM6_Z5_1 | 5 | 169931570 | 180687824 | 0.0522 | 0.9727 | 1.9895 | 1.9356 | 2 | 2 |
| WCM6_Z5_1 | 6 | 292582 | 57512678 | 0.0495 | 0.9699 | 1.9918 | 1.9256 | 2 | 2 |
| WCM6_Z5_1 | 6 | 62390958 | 102483365 | −0.8242 | 0.0264 | 2.0103 | 0.0267 | 2 | 0 |
| WCM6_Z5_1 | 6 | 102483435 | 129571329 | −1.5713 | −0.8317 | 1.0848 | 0.039 | 1 | 0 |
| WCM6_Z5_1 | 6 | 129573432 | 138734011 | −0.3215 | 0.5747 | 1.8959 | 1.0828 | 2 | 1 |
| WCM6_Z5_1 | 6 | 139094822 | 170893408 | 0.0383 | 0.958 | 1.9632 | 1.9221 | 2 | 2 |
| WCM6_Z5_1 | 7 | 193717 | 28610032 | 0.357 | 1.293 | 2.8884 | 2.0124 | 3 | 2 |
| WCM6_Z5_1 | 7 | 28725796 | 39612224 | −0.8555 | −0.0083 | 1.957 | 0.0315 | 2 | 0 |
| WCM6_Z5_1 | 7 | 39726346 | 76071147 | 0.3582 | 1.2943 | 2.9729 | 1.9321 | 3 | 2 |
| WCM6_Z5_1 | 7 | 76109950 | 82595650 | 1.3846 | 2.3536 | 7.2571 | 2.9646 | 7 | 3 |
| WCM6_Z5_1 | 7 | 82763817 | 87856203 | −0.3344 | 0.5609 | 2.9488 | 0.0015 | 3 | 0 |
| WCM6_Z5_1 | 7 | 87908920 | 158937416 | 0.0561 | 0.9768 | 2.8809 | 1.0553 | 3 | 1 |
| WCM6_Z5_1 | 8 | 190928 | 1514009 | 0.1715 | 1.0985 | NA | NA | NA | NA |
| WCM6_Z5_1 | 8 | 1574933 | 3611497 | 0.0373 | 0.957 | NA | NA | NA | NA |
| WCM6_Z5_1 | 8 | 3855632 | 33354233 | −0.8141 | 0.0376 | 2.0263 | 0.0265 | 2 | 0 |
| WCM6_Z5_1 | 8 | 33356074 | 33454885 | −0.7491 | 0.1095 | NA | NA | NA | NA |
| WCM6_Z5_1 | 8 | 35406917 | 40011362 | 0.1185 | 1.0427 | 2.2185 | 1.9015 | 2 | 2 |
| WCM6_Z5_1 | 8 | 40389730 | 42857247 | −0.6946 | 0.1695 | 2.201 | 0.0483 | 2 | 0 |
| WCM6_Z5_1 | 8 | 42862997 | 146279472 | 1.2828 | 2.2495 | 7.3585 | 2.1517 | 7 | 2 |
| WCM6_Z5_1 | 9 | 17044 | 71152313 | 0.0428 | 0.9628 | 1.9739 | 1.9244 | 2 | 2 |
| WCM6_Z5_1 | 9 | 71155621 | 141071320 | 0.3502 | 1.2859 | 2.9074 | 1.9692 | 3 | 2 |
| WCM6_Z5_1 | X | 2707687 | 57936541 | 0.5524 | 1.4967 | NA | NA | NA | NA |
| WCM6_Z5_1 | X | 62898360 | 69282554 | 1.5282 | 2.5002 | NA | NA | NA | NA |
| WCM6_Z5_1 | X | 69282680 | 154509348 | 0.5528 | 1.4971 | NA | NA | NA | NA |
| WCM7 | 1 | 14930 | 1149126 | 0.0356 | 0.155 | 1.3231 | 0.9038 | 1 | 1 |
| WCM7 | 1 | 1149559 | 145475378 | −0.1576 | −0.9167 | 0.6153 | 0.4441 | 1 | 0 |
| WCM7 | 1 | 145487375 | 249214028 | 0.0509 | 0.2182 | 1.1633 | 1.1633 | 1 | 1 |
| WCM7 | 10 | 226048 | 55943305 | 0.0295 | 0.1295 | 1.0939 | 1.0939 | 1 | 1 |
| WCM7 | 10 | 55943429 | 96466633 | −0.1856 | −1.1479 | 0.5112 | 0.3913 | 1 | 0 |
| WCM7 | 10 | 96466720 | 135477891 | 0.0499 | 0.2142 | 1.16 | 1.16 | 1 | 1 |
| WCM7 | 11 | 193096 | 134605916 | 0.0509 | 0.2183 | 1.1633 | 1.1633 | 1 | 1 |
| WCM7 | 12 | 148468 | 133810868 | 0.0361 | 0.1571 | 1.115 | 1.115 | 1 | 1 |
| WCM7 | 13 | 19042044 | 115089411 | −0.1923 | −1.2083 | 0.5123 | 0.3532 | 1 | 0 |
| WCM7 | 14 | 19110239 | 107283274 | 0.0291 | 0.1276 | 1.0925 | 1.0925 | 1 | 1 |
| WCM7 | 15 | 20083896 | 102515245 | −0.1566 | −0.9092 | 0.614 | 0.4509 | 1 | 0 |
| WCM7 | 16 | 66894 | 304514 | −0.1045 | −0.5513 | NA | NA | NA | NA |
| WCM7 | 16 | 304684 | 50248191 | 0.0353 | 0.1539 | 1.1126 | 1.1126 | 1 | 1 |
| WCM7 | 16 | 50250043 | 90260461 | −0.1578 | −0.9189 | 0.6104 | 0.4474 | 1 | 0 |
| WCM7 | 17 | 131595 | 81187839 | 0.021 | 0.093 | 1.0666 | 1.0666 | 1 | 1 |
| WCM7 | 18 | 47824 | 23969850 | 0.0185 | 0.0822 | 1.0586 | 1.0586 | 1 | 1 |
| WCM7 | 18 | 23970027 | 74593283 | −0.1749 | −1.0558 | 0.5575 | 0.4045 | 1 | 0 |
| WCM7 | 18 | 74611127 | 78005281 | −0.0644 | −0.3191 | 0.8015 | 0.8015 | 1 | 1 |
| WCM7 | 19 | 105041 | 59093696 | 0.0422 | 0.1825 | 1.1349 | 1.1349 | 1 | 1 |
| WCM7 | 2 | 41583 | 243037022 | 0.038 | 0.1654 | 2.2415 | 0.0014 | 2 | 0 |
| WCM7 | 20 | 68337 | 62934592 | 0.0419 | 0.1813 | 1.1339 | 1.1339 | 1 | 1 |
| WCM7 | 21 | 9590311 | 48084201 | −0.0149 | −0.0688 | 0.9534 | 0.9534 | 1 | 1 |
| WCM7 | 22 | 16159068 | 51219129 | −0.1497 | −0.8572 | 0.6381 | 0.466 | 1 | 0 |
| WCM7 | 3 | 239531 | 131254111 | 0.0441 | 0.1904 | 1.1411 | 1.1411 | 1 | 1 |
| WCM7 | 3 | 131261473 | 197896660 | 0.2049 | 0.76 | 1.6935 | 1.6935 | 2 | 2 |
| WCM7 | 4 | 53378 | 40763142 | −0.1887 | −1.1752 | 0.5226 | 0.3631 | 1 | 0 |
| WCM7 | 4 | 40776459 | 190904471 | 0.0101 | 0.0451 | 1.0318 | 1.0318 | 1 | 1 |
| WCM7 | 5 | 90714 | 52404312 | 0.1678 | 0.6421 | 1.5606 | 1.5606 | 2 | 2 |
| WCM7 | 5 | 52404546 | 133996832 | −0.0597 | −0.2937 | 1.4395 | 0.1922 | 1 | 0 |
| WCM7 | 5 | 133997136 | 180687821 | 0.1527 | 0.592 | 1.5074 | 1.5074 | 2 | 2 |
| WCM7 | 6 | 203542 | 63939127 | 0.0296 | 0.1298 | 1.0941 | 1.0941 | 1 | 1 |
| WCM7 | 6 | 63939458 | 170892848 | −0.1832 | −1.1267 | 0.538 | 0.3779 | 1 | 0 |
| WCM7 | 7 | 195638 | 158937022 | 0.2098 | 0.7752 | 1.7115 | 1.7115 | 2 | 2 |
| WCM7 | 8 | 163476 | 36642059 | −0.1584 | −0.923 | 0.6089 | 0.446 | 1 | 0 |
| WCM7 | 8 | 36644852 | 48689573 | 0.0347 | 0.1516 | 1.1108 | 1.1108 | 1 | 1 |
| WCM7 | 8 | 48690265 | 125339650 | 0.2148 | 0.7905 | 1.7297 | 1.7297 | 2 | 2 |
| WCM7 | 8 | 125342947 | 145737040 | 0.0917 | 0.3767 | 1.2984 | 1.2984 | 1 | 1 |
| WCM7 | 8 | 145737286 | 146279332 | −0.0114 | −0.0524 | 1.0726 | 0.8561 | 1 | 1 |
| WCM7 | 9 | 15908 | 15267878 | 0.0475 | 0.2044 | 1.1522 | 1.1522 | 1 | 1 |
| WCM7 | 9 | 15307171 | 27949716 | −0.1449 | −0.8221 | 0.652 | 0.4793 | 1 | 0 |
| WCM7 | 9 | 27949961 | 141107539 | 0.0505 | 0.2167 | 1.1621 | 1.1621 | 1 | 1 |
| WCM7 | X | 2700084 | 154493601 | 0.048 | 0.2063 | NA | NA | NA | NA |
| WCM73 | 1 | 17483 | 249213964 | 0.0272 | 0.8473 | 1.7992 | 1.7992 | 2 | 2 |
| WCM73 | 10 | 225914 | 135438935 | 0 | 0.2175 | 1.1627 | 1.1627 | 1 | 1 |
| WCM73 | 11 | 193096 | 134605906 | 0.0083 | 0.4386 | 1.3553 | 1.3553 | 1 | 1 |
| WCM73 | 12 | 148417 | 133810752 | −1.00E−04 | 0.2146 | 1.1603 | 1.1603 | 1 | 1 |
| WCM73 | 13 | 19041979 | 115089411 | −0.0258 | −0.8169 | 0.5676 | 0.5676 | 1 | 1 |
| WCM73 | 14 | 19110239 | 107283263 | −0.0532 | NA | −0.0496 | −0.0496 | 0 | 0 |
| WCM73 | 15 | 20083896 | 102514227 | 0.0306 | 0.91 | 1.879 | 1.879 | 2 | 2 |
| WCM73 | 16 | 67604 | 90168701 | −0.0286 | −0.9847 | 0.5053 | 0.5053 | 1 | 1 |
| WCM73 | 17 | 131595 | 81187925 | 2.00E−04 | 0.2232 | 1.1673 | 1.1673 | 1 | 1 |
| WCM73 | 18 | 47684 | 78005224 | −0.0611 | NA | −0.2263 | −0.2263 | 0 | 0 |
| WCM73 | 19 | 104963 | 59093645 | −0.0304 | −1.1122 | 0.4626 | 0.4626 | 0 | 0 |
| WCM73 | 2 | 41509 | 243036990 | 0.0075 | 0.4195 | 1.3375 | 1.3375 | 1 | 1 |
| WCM73 | 20 | 68373 | 62934623 | 0.014 | 0.575 | 1.4897 | 1.4897 | 1 | 1 |
| WCM73 | 21 | 9590311 | 48084136 | 0.0079 | 0.4288 | 1.3461 | 1.3461 | 1 | 1 |
| WCM73 | 22 | 16159016 | 51219090 | 0.0081 | 0.4326 | 1.3497 | 1.3497 | 1 | 1 |
| WCM73 | 3 | 239530 | 197896818 | −0.0026 | 0.141 | 1.1027 | 1.1027 | 1 | 1 |
| WCM73 | 4 | 53363 | 190903980 | −0.0106 | −0.126 | 0.9164 | 0.9164 | 1 | 1 |
| WCM73 | 5 | 90697 | 180687818 | −0.0218 | −0.6023 | 0.6587 | 0.6587 | 1 | 1 |
| WCM73 | 6 | 203369 | 170892848 | −0.0217 | −0.5953 | 0.6619 | 0.6619 | 1 | 1 |
| WCM73 | 7 | 193253 | 158937011 | −0.0089 | −0.066 | 0.9553 | 0.9553 | 1 | 1 |
| WCM73 | 8 | 163476 | 146279423 | −0.0283 | −0.9661 | 0.5119 | 0.5119 | 1 | 1 |
| WCM73 | 9 | 14884 | 141107514 | 0.0089 | 0.4547 | 1.3705 | 1.3705 | 1 | 1 |
| WCM73 | X | 2700189 | 154493484 | −0.2189 | NA | NA | NA | NA | NA |
| WCM73_X1_1 | 1 | 69590 | 26644555 | 0.0166 | 0.0272 | 1.0449 | 0.9931 | 1 | 1 |
| WCM73_X1_1 | 1 | 26646717 | 27744862 | −0.2484 | −0.8983 | NA | NA | NA | NA |
| WCM73_X1_1 | 1 | 27745553 | 249212420 | 0.0199 | 0.0366 | 1.043 | 1.0084 | 1 | 1 |
| WCM73_X1_1 | 10 | 225958 | 135440108 | 0.0091 | 0.0059 | 1.0041 | 1.0041 | 1 | 1 |
| WCM73_X1_1 | 11 | 193127 | 2016825 | 0.0362 | 0.0819 | 1.6095 | 0.5074 | 2 | 1 |
| WCM73_X1_1 | 11 | 2017128 | 3381034 | −0.1958 | −0.6772 | 0.8016 | 0.4491 | 1 | 0 |
| WCM73_X1_1 | 11 | 3381337 | 134856603 | 0.0272 | 0.0568 | 1.0402 | 1.0402 | 1 | 1 |
| WCM73_X1_1 | 12 | 148811 | 133810842 | 0.0239 | 0.0478 | 1.0424 | 1.025 | 1 | 1 |
| WCM73_X1_1 | 13 | 19600364 | 115089411 | −0.0157 | −0.0657 | 1.0061 | 0.9049 | 1 | 1 |
| WCM73_X1_1 | 14 | 19377716 | 55903285 | 0.0072 | 7.00E−04 | 1.0319 | 0.9691 | 1 | 1 |
| WCM73_X1_1 | 14 | 55903640 | 79117616 | −0.2514 | −0.912 | 1.0569 | 0.006 | 1 | 0 |
| WCM73_X1_1 | 14 | 79175749 | 107283091 | −0.0147 | −0.0627 | 1.0081 | 0.9068 | 1 | 1 |
| WCM73_X1_1 | 15 | 20170036 | 102517666 | −0.0596 | −0.1992 | 0.9011 | 0.841 | 1 | 1 |
| WCM73_X1_1 | 16 | 67604 | 50060368 | 0.02 | 0.0369 | 1.0259 | 1.0259 | 1 | 1 |
| WCM73_X1_1 | 16 | 50063666 | 55559524 | −0.2502 | −0.9065 | 1.1694 | −0.1024 | 1 | 0 |
| WCM73_X1_1 | 16 | 55562359 | 79633459 | 0.023 | 0.0452 | 1.0578 | 1.0058 | 1 | 1 |
| WCM73_X1_1 | 16 | 79633797 | 90233622 | −0.2105 | −0.7366 | 1.0203 | 0.1799 | 1 | 0 |
| WCM73_X1_1 | 17 | 131595 | 81083567 | −0.0011 | −0.0231 | 1.0097 | 0.9585 | 1 | 1 |
| WCM73_X1_1 | 18 | 48224 | 78005224 | −0.2295 | −0.8162 | 1.0578 | 0.0781 | 1 | 0 |
| WCM73_X1_1 | 19 | 281507 | 1149368 | 0.0352 | 0.0792 | 1.0943 | 1.0186 | 1 | 1 |
| WCM73_X1_1 | 19 | 1154193 | 3902518 | −0.1405 | −0.4692 | 1.0783 | 0.3664 | 1 | 0 |
| WCM73_X1_1 | 19 | 3905633 | 11364354 | 0.0201 | 0.0371 | 1.0433 | 1.0087 | 1 | 1 |
| WCM73_X1_1 | 19 | 11373088 | 13054676 | −0.274 | −1.0166 | 1.0045 | −0.016 | 1 | 0 |
| WCM73_X1_1 | 19 | 13056799 | 59083929 | −0.0246 | −0.0922 | 0.9661 | 0.9101 | 1 | 1 |
| WCM73_X1_1 | 2 | 41613 | 1312299 | −0.1101 | −0.3635 | NA | NA | NA | NA |
| WCM73_X1_1 | 2 | 1320143 | 96781886 | 2.00E−04 | −0.0196 | 0.9865 | 0.9865 | 1 | 1 |
| WCM73_X1_1 | 2 | 96789787 | 232790241 | −7.00E−04 | −0.0221 | 1.9112 | 0.0583 | 2 | 0 |
| WCM73_X1_1 | 2 | 232879652 | 233995353 | −0.4741 | −2.3971 | NA | NA | NA | NA |
| WCM73_X1_1 | 2 | 233995535 | 242946607 | −6.00E−04 | −0.0218 | 1.9315 | 0.0386 | 2 | 0 |
| WCM73_X1_1 | 20 | 68351 | 62904835 | −0.005 | −0.0347 | 1.0074 | 0.9451 | 1 | 1 |
| WCM73_X1_1 | 21 | 9907279 | 48084277 | −0.0933 | −0.3075 | 0.848 | 0.7681 | 1 | 1 |
| WCM73_X1_1 | 22 | 16287431 | 51219138 | −0.0177 | −0.0718 | 0.9711 | 0.9318 | 1 | 1 |
| WCM73_X1_1 | 3 | 239347 | 141644446 | 0.0263 | 0.0545 | 1.0559 | 1.0211 | 1 | 1 |
| WCM73_X1_1 | 3 | 141671431 | 146303867 | −0.2568 | −0.9362 | NA | NA | NA | NA |
| WCM73_X1_1 | 3 | 146307533 | 197894712 | 0.0256 | 0.0524 | 1.0717 | 1.0023 | 1 | 1 |
| WCM73_X1_1 | 4 | 59331 | 190426502 | −0.0286 | −0.1042 | 0.9526 | 0.908 | 1 | 1 |
| WCM73_X1_1 | 5 | 140560 | 54252345 | 0.0252 | 0.0513 | 1.1056 | 0.9667 | 1 | 1 |
| WCM73_X1_1 | 5 | 54253581 | 79949874 | −0.1838 | −0.6302 | 1.0716 | 0.2205 | 1 | 0 |
| WCM73_X1_1 | 5 | 79950403 | 180687773 | 0.0276 | 0.0581 | 1.0643 | 1.0179 | 1 | 1 |
| WCM73_X1_1 | 6 | 203699 | 170893661 | −0.0185 | −0.0742 | 0.9667 | 0.9331 | 1 | 1 |
| WCM73_X1_1 | 7 | 193253 | 149133916 | 0.0179 | 0.0309 | 1.0648 | 0.9784 | 1 | 1 |
| WCM73_X1_1 | 7 | 149148163 | 155604655 | −0.2271 | −0.8056 | 1.0774 | 0.0668 | 1 | 0 |
| WCM73_X1_1 | 7 | 155748043 | 158937142 | 0.0226 | 0.044 | 1.0858 | 0.9761 | 1 | 1 |
| WCM73_X1_1 | 8 | 190900 | 24251645 | −0.2372 | −0.849 | 1.1317 | −0.0214 | 1 | 0 |
| WCM73_X1_1 | 8 | 24253290 | 28420320 | 0.011 | 0.0115 | 1.0567 | 0.9593 | 1 | 1 |
| WCM73_X1_1 | 8 | 28573760 | 104417045 | −0.0504 | −0.1705 | 0.9379 | 0.8391 | 1 | 1 |
| WCM73_X1_1 | 8 | 104420009 | 146279489 | 0.0323 | 0.071 | 1.0766 | 1.0243 | 1 | 1 |
| WCM73_X1_1 | 9 | 17055 | 134385788 | 0.0259 | 0.0533 | 1.055 | 1.0203 | 1 | 1 |
| WCM73_X1_1 | 9 | 134386759 | 137309131 | −0.2653 | −0.9757 | 1.0573 | −0.0403 | 1 | 0 |
| WCM73_X1_1 | 9 | 137313635 | 141111414 | 0.0059 | −0.0031 | 1.0264 | 0.9693 | 1 | 1 |
| WCM73_X1_1 | X | 2707682 | 154509348 | −0.0081 | −0.0434 | NA | NA | NA | NA |
| WCM78 | 1 | 14930 | 898735 | −0.089 | −0.477 | NA | NA | NA | NA |
| WCM78 | 1 | 898872 | 16804753 | 0.0787 | 0.4527 | 1.3686 | 1.3686 | 1 | 1 |
| WCM78 | 1 | 16804965 | 150040828 | −0.041 | −0.1568 | 0.897 | 0.897 | 1 | 1 |
| WCM78 | 1 | 150044243 | 152127631 | 0.4244 | 1.5718 | 4.3279 | 1.6177 | 4 | 2 |
| WCM78 | 1 | 152127741 | 249213973 | −0.0212 | −0.0407 | 0.9722 | 0.9722 | 1 | 1 |
| WCM78 | 10 | 225914 | 106153158 | −0.0554 | −0.2467 | 0.8428 | 0.8428 | 1 | 1 |
| WCM78 | 10 | 106153254 | 135438955 | 0.0995 | 0.5406 | 1.4546 | 1.4546 | 1 | 1 |
| WCM78 | 11 | 193096 | 62291779 | 0.0111 | 0.134 | 1.103 | 1.0917 | 1 | 1 |
| WCM78 | 11 | 62292063 | 62678147 | −0.2651 | −3.1533 | 0.1684 | 0.0564 | 0 | 0 |
| WCM78 | 11 | 62678512 | 134320624 | 0.0403 | 0.2787 | 1.2131 | 1.2131 | 1 | 1 |
| WCM78 | 12 | 148444 | 310983 | 0.0868 | 0.4874 | NA | NA | NA | NA |
| WCM78 | 12 | 312021 | 31254752 | 0.2886 | 1.1995 | 2.2965 | 2.2965 | 2 | 2 |
| WCM78 | 12 | 31255238 | 57146122 | 0.0547 | 0.3461 | 1.2711 | 1.2711 | 1 | 1 |
| WCM78 | 12 | 57167649 | 62768361 | 0.3208 | 1.2934 | 3.46 | 1.4421 | 3 | 1 |
| WCM78 | 12 | 62775413 | 116413409 | 0.1037 | 0.5581 | 1.4723 | 1.4723 | 1 | 1 |
| WCM78 | 12 | 116418505 | 122208890 | 0.4356 | 1.5999 | 4.4576 | 1.605 | 4 | 2 |
| WCM78 | 12 | 122209503 | 133810703 | −0.066 | −0.3158 | 0.9534 | 0.6533 | 1 | 1 |
| WCM78 | 13 | 19042044 | 109282133 | −0.0264 | −0.0702 | 0.9525 | 0.9525 | 1 | 1 |
| WCM78 | 13 | 109311775 | 111340117 | 0.3559 | 1.3917 | 2.6239 | 2.6239 | 3 | 3 |
| WCM78 | 13 | 111340386 | 115089411 | 0.0031 | 0.0928 | 1.0664 | 1.0664 | 1 | 1 |
| WCM78 | 14 | 19110378 | 22038268 | −0.0431 | −0.1699 | 0.8889 | 0.8889 | 1 | 1 |
| WCM78 | 14 | 22038456 | 24114450 | 0.3544 | 1.3876 | 3.8571 | 1.3756 | 4 | 1 |
| WCM78 | 14 | 24403717 | 107283266 | 0.0037 | 0.0955 | 1.0684 | 1.0684 | 1 | 1 |
| WCM78 | 15 | 20083896 | 102506273 | 0.017 | 0.1644 | 1.1207 | 1.1207 | 1 | 1 |
| WCM78 | 16 | 64423 | 740365 | −0.0166 | −0.0148 | 1.0841 | 0.8955 | 1 | 1 |
| WCM78 | 16 | 742668 | 2474346 | 0.1551 | 0.7576 | 1.8281 | 1.5533 | 2 | 2 |
| WCM78 | 16 | 2474680 | 90168855 | −0.0344 | −0.1173 | 0.9219 | 0.9219 | 1 | 1 |
| WCM78 | 17 | 131595 | 6913781 | −0.0544 | −0.2406 | 0.8464 | 0.8464 | 1 | 1 |
| WCM78 | 17 | 6915497 | 8132135 | −0.3084 | NA | 0.0061 | −0.0573 | 0 | 0 |
| WCM78 | 17 | 8132489 | 81187834 | 0.0096 | 0.1264 | 1.0916 | 1.0916 | 1 | 1 |
| WCM78 | 18 | 47804 | 78005210 | 0.0042 | 0.0985 | 1.0707 | 1.0707 | 1 | 1 |
| WCM78 | 19 | 105021 | 2901091 | −0.1677 | −1.1887 | 0.5386 | 0.3388 | 1 | 0 |
| WCM78 | 19 | 2915598 | 5215310 | 0.1654 | 0.7955 | 1.742 | 1.7294 | 2 | 2 |
| WCM78 | 19 | 5215558 | 50453416 | −0.0295 | −0.0883 | 0.9406 | 0.9406 | 1 | 1 |
| WCM78 | 19 | 50455177 | 52505036 | 0.26 | 1.1123 | 3.1292 | 1.1946 | 3 | 1 |
| WCM78 | 19 | 52505463 | 59093645 | −0.0488 | −0.2051 | 0.8675 | 0.8675 | 1 | 1 |
| WCM78 | 2 | 41662 | 3628529 | −0.0984 | −0.5476 | 0.6842 | 0.6842 | 1 | 1 |
| WCM78 | 2 | 3651221 | 204736155 | 0.0057 | 0.1064 | 1.0765 | 1.0765 | 1 | 1 |
| WCM78 | 2 | 204737487 | 243037022 | −0.1058 | −0.6049 | 0.6575 | 0.6575 | 1 | 1 |
| WCM78 | 20 | 68336 | 62934572 | −0.0554 | −0.247 | 0.8427 | 0.8427 | 1 | 1 |
| WCM78 | 21 | 9590271 | 48084214 | −0.017 | −0.0169 | 0.9883 | 0.9883 | 1 | 1 |
| WCM78 | 22 | 16159005 | 51219138 | −0.0123 | 0.0094 | 1.0066 | 1.0066 | 1 | 1 |
| WCM78 | 3 | 239524 | 65940205 | 0.004 | 0.0971 | 1.0697 | 1.0697 | 1 | 1 |
| WCM78 | 3 | 66017711 | 69435184 | 0.4374 | 1.6046 | 3.4437 | 2.6385 | 3 | 3 |
| WCM78 | 3 | 69788724 | 197896683 | −0.0061 | 0.0434 | 1.0305 | 1.0305 | 1 | 1 |
| WCM78 | 4 | 53418 | 70823000 | −0.0333 | −0.1109 | 0.926 | 0.926 | 1 | 1 |
| WCM78 | 4 | 70823265 | 74853890 | 0.2043 | 0.9316 | 2.0431 | 1.7717 | 2 | 2 |
| WCM78 | 4 | 74861637 | 190904482 | −0.0369 | −0.1319 | 0.9126 | 0.9126 | 1 | 1 |
| WCM78 | 5 | 90680 | 180687803 | −0.031 | −0.0971 | 0.9349 | 0.9349 | 1 | 1 |
| WCM78 | 6 | 203535 | 27806861 | −0.0602 | −0.2779 | 0.8248 | 0.8248 | 1 | 1 |
| WCM78 | 6 | 27809368 | 32007956 | 0.1912 | 0.8868 | 3.0404 | 0.6578 | 3 | 1 |
| WCM78 | 6 | 32016415 | 144869844 | 0.0236 | 0.1976 | 1.1468 | 1.1468 | 1 | 1 |
| WCM78 | 6 | 144870019 | 148872433 | 0.1797 | 0.8467 | 2.2241 | 1.3727 | 2 | 1 |
| WCM78 | 6 | 148872898 | 170892848 | −0.0862 | −0.456 | 0.729 | 0.729 | 1 | 1 |
| WCM78 | 7 | 193222 | 30066104 | 0.1529 | 0.7497 | 1.6814 | 1.6814 | 2 | 2 |
| WCM78 | 7 | 30084684 | 33014377 | 0.4277 | 1.5803 | 4.522 | 1.4584 | 5 | 1 |
| WCM78 | 7 | 33014963 | 49951754 | 0.1465 | 0.7257 | 1.6599 | 1.6475 | 2 | 2 |
| WCM78 | 7 | 49952114 | 148113783 | 0.0175 | 0.1667 | 1.1282 | 1.1168 | 1 | 1 |
| WCM78 | 7 | 148114529 | 151064006 | 0.3048 | 1.2473 | 2.5611 | 2.1869 | 3 | 2 |
| WCM78 | 7 | 151064228 | 158937227 | −0.1296 | −0.8056 | 0.8285 | 0.3157 | 1 | 0 |
| WCM78 | 8 | 163470 | 99208130 | −0.0094 | 0.0254 | 1.0178 | 1.0178 | 1 | 1 |
| WCM78 | 8 | 99215285 | 126163426 | 0.1389 | 0.6969 | 1.6211 | 1.6211 | 2 | 2 |
| WCM78 | 8 | 126194510 | 128098838 | 0.0054 | 0.1046 | NA | NA | NA | NA |
| WCM78 | 8 | 128428061 | 146003567 | 0.3996 | 1.5082 | 3.8806 | 1.8085 | 4 | 2 |
| WCM78 | 8 | 146005624 | 146279431 | 0.1378 | 0.6928 | 2.32 | 0.9128 | 2 | 1 |
| WCM78 | 9 | 14887 | 141107545 | −0.0069 | 0.0391 | 1.0275 | 1.0275 | 1 | 1 |
| WCM78 | X | 2700151 | 11072078 | −0.0392 | −0.1456 | NA | NA | NA | NA |
| WCM78 | X | 11072297 | 29414436 | 0.3165 | 1.281 | NA | NA | NA | NA |
| WCM78 | X | 29417363 | 133304174 | −0.0265 | −0.0707 | NA | NA | NA | NA |
| WCM78 | X | 133372863 | 134126394 | 0.7069 | 2.202 | NA | NA | NA | NA |
| WCM78 | X | 134155927 | 154493585 | −0.0652 | −0.3108 | NA | NA | NA | NA |
| WCM78_Z1_1 | 1 | 721439 | 878434 | −0.0261 | 0.148 | NA | NA | NA | NA |
| WCM78_Z1_1 | 1 | 878716 | 2938265 | 0.2213 | 0.5018 | 1.8889 | 0.943 | 2 | 1 |
| WCM78_Z1_1 | 1 | 2938692 | 16578147 | 0.0481 | 0.2569 | 1.4012 | 0.9887 | 1 | 1 |
| WCM78_Z1_1 | 1 | 16578863 | 29652156 | −0.1607 | −0.0568 | 0.9794 | 0.9434 | 1 | 1 |
| WCM78_Z1_1 | 1 | 29696230 | 35579769 | 0.0981 | 0.3289 | 1.554 | 0.9582 | 2 | 1 |
| WCM78_Z1_1 | 1 | 35580079 | 39466695 | −0.1556 | −0.049 | 1.1216 | 0.8116 | 1 | 1 |
| WCM78_Z1_1 | 1 | 39469068 | 40211140 | 0.1825 | 0.4481 | NA | NA | NA | NA |
| WCM78_Z1_1 | 1 | 40214692 | 65068566 | −0.1462 | −0.0344 | 1.0007 | 0.9522 | 1 | 1 |
| WCM78_Z1_1 | 1 | 65095077 | 67891937 | 0.224 | 0.5055 | NA | NA | NA | NA |
| WCM78_Z1_1 | 1 | 67895918 | 116380979 | −0.1388 | −0.0228 | 1.0148 | 0.9539 | 1 | 1 |
| WCM78_Z1_1 | 1 | 116519280 | 118166515 | 0.2828 | 0.586 | 2.011 | 0.9912 | 2 | 1 |
| WCM78_Z1_1 | 1 | 118413224 | 149877566 | −0.077 | 0.0716 | 1.3831 | 0.7187 | 1 | 1 |
| WCM78_Z1_1 | 1 | 149878293 | 152129173 | 0.9724 | 1.46 | 4.7742 | 0.728 | 5 | 1 |
| WCM78_Z1_1 | 1 | 152129428 | 154698440 | −0.1665 | −0.0659 | 0.9897 | 0.921 | 1 | 1 |
| WCM78_Z1_1 | 1 | 154709546 | 155172907 | 0.2163 | 0.495 | NA | NA | NA | NA |
| WCM78_Z1_1 | 1 | 155173301 | 196197439 | −0.1074 | 0.0255 | 1.052 | 0.9836 | 1 | 1 |
| WCM78_Z1_1 | 1 | 196205140 | 203472834 | 0.0844 | 0.3093 | 1.5093 | 0.9689 | 2 | 1 |
| WCM78_Z1_1 | 1 | 203652473 | 204588916 | 0.4525 | 0.812 | NA | NA | NA | NA |
| WCM78_Z1_1 | 1 | 204589072 | 207758176 | 0.0284 | 0.2283 | 1.3116 | 1.0312 | 1 | 1 |
| WCM78_Z1_1 | 1 | 207760874 | 211093346 | −0.2016 | −0.1212 | 0.9967 | 0.8421 | 1 | 1 |
| WCM78_Z1_1 | 1 | 211192166 | 212798977 | 0.2027 | 0.4761 | NA | NA | NA | NA |
| WCM78_Z1_1 | 1 | 212799330 | 241094033 | −0.2047 | −0.126 | 0.9323 | 0.9003 | 1 | 1 |
| WCM78_Z1_1 | 1 | 241099999 | 243327896 | 0.165 | 0.4235 | NA | NA | NA | NA |
| WCM78_Z1_1 | 1 | 243328842 | 249212434 | −0.1909 | −0.1043 | 0.9508 | 0.9097 | 1 | 1 |
| WCM78_Z1_1 | 10 | 225952 | 19612972 | −0.4025 | −0.4542 | 0.9696 | 0.4902 | 1 | 0 |
| WCM78_Z1_1 | 10 | 19616498 | 69792546 | −0.1308 | −0.0105 | 1.0142 | 0.9713 | 1 | 1 |
| WCM78_Z1_1 | 10 | 69793903 | 70922249 | 0.2354 | 0.5213 | NA | NA | NA | NA |
| WCM78_Z1_1 | 10 | 70925853 | 106960956 | −0.1444 | −0.0315 | 0.983 | 0.9739 | 1 | 1 |
| WCM78_Z1_1 | 10 | 106970952 | 107022174 | 0.0544 | 0.266 | NA | NA | NA | NA |
| WCM78_Z1_1 | 10 | 108337227 | 119302836 | 0.2792 | 0.5812 | 1.7768 | 1.2153 | 2 | 1 |
| WCM78_Z1_1 | 10 | 119303155 | 135346340 | 0.0502 | 0.2599 | 1.3744 | 1.0203 | 1 | 1 |
| WCM78_Z1_1 | 10 | 135347380 | 135440090 | −0.0345 | 0.1355 | NA | NA | NA | NA |
| WCM78_Z1_1 | 11 | 193127 | 2016741 | −0.1689 | −0.0696 | 1.2154 | 0.6904 | 1 | 1 |
| WCM78_Z1_1 | 11 | 2017122 | 2591932 | 0.2475 | 0.5379 | NA | NA | NA | NA |
| WCM78_Z1_1 | 11 | 2592619 | 4621764 | −0.0813 | 0.0652 | 1.1286 | 0.9638 | 1 | 1 |
| WCM78_Z1_1 | 11 | 4622015 | 5323171 | 0.2988 | 0.6078 | 2.1034 | 0.9444 | 2 | 1 |
| WCM78_Z1_1 | 11 | 5344907 | 46625353 | −0.1367 | −0.0196 | 1.0094 | 0.9637 | 1 | 1 |
| WCM78_Z1_1 | 11 | 46637197 | 48328695 | 0.3661 | 0.6981 | NA | NA | NA | NA |
| WCM78_Z1_1 | 11 | 48346681 | 63766449 | −0.1393 | −0.0236 | 1.0096 | 0.9579 | 1 | 1 |
| WCM78_Z1_1 | 11 | 63766819 | 66481851 | 0.2482 | 0.5388 | 1.9545 | 0.951 | 2 | 1 |
| WCM78_Z1_1 | 11 | 66482814 | 67399334 | 0.6682 | 1.088 | NA | NA | NA | NA |
| WCM78_Z1_1 | 11 | 67399798 | 69063384 | 0.2632 | 0.5593 | NA | NA | NA | NA |
| WCM78_Z1_1 | 11 | 69063594 | 75152243 | −0.1596 | −0.0552 | 0.9955 | 0.9294 | 1 | 1 |
| WCM78_Z1_1 | 11 | 75152805 | 88924375 | 0.3157 | 0.6306 | 2.095 | 1.0015 | 2 | 1 |
| WCM78_Z1_1 | 11 | 88925201 | 116629800 | −0.1465 | −0.0348 | 1.017 | 0.9353 | 1 | 1 |
| WCM78_Z1_1 | 11 | 116631510 | 119340576 | 0.3018 | 0.6118 | 1.9457 | 1.1107 | 2 | 1 |
| WCM78_Z1_1 | 11 | 119369420 | 134856556 | −0.1131 | 0.0168 | 1.0489 | 0.9745 | 1 | 1 |
| WCM78_Z1_1 | 12 | 148639 | 14019098 | 0.4356 | 0.7899 | 2.4024 | 1.0556 | 2 | 1 |
| WCM78_Z1_1 | 12 | 14576917 | 19671645 | 0.9662 | 1.4526 | 4.0866 | 1.3876 | 4 | 1 |
| WCM78_Z1_1 | 12 | 20522359 | 34182559 | 0.4111 | 0.7578 | 2.2527 | 1.1291 | 2 | 1 |
| WCM78_Z1_1 | 12 | 38710697 | 57106677 | −0.0103 | 0.1714 | 1.1627 | 1.0895 | 1 | 1 |
| WCM78_Z1_1 | 12 | 57106972 | 60173262 | 0.419 | 0.7682 | 2.0954 | 1.3109 | 2 | 1 |
| WCM78_Z1_1 | 12 | 62104181 | 69646914 | −8.00E−04 | 0.1855 | 1.226 | 1.0484 | 1 | 1 |
| WCM78_Z1_1 | 12 | 69650492 | 77216256 | 0.3718 | 0.7057 | 2.1103 | 1.1515 | 2 | 1 |
| WCM78_Z1_1 | 12 | 77222173 | 93175790 | −0.0124 | 0.1683 | 1.1985 | 1.0489 | 1 | 1 |
| WCM78_Z1_1 | 12 | 93181739 | 95771114 | 0.3807 | 0.7176 | NA | NA | NA | NA |
| WCM78_Z1_1 | 12 | 95774646 | 115801955 | −0.005 | 0.1792 | 1.1573 | 1.1072 | 1 | 1 |
| WCM78_Z1_1 | 12 | 116399156 | 121341672 | 0.7824 | 1.2296 | 3.1784 | 1.5117 | 3 | 2 |
| WCM78_Z1_1 | 12 | 121416650 | 122265949 | 0.3894 | 0.7291 | 2.8742 | 0.441 | 3 | 0 |
| WCM78_Z1_1 | 12 | 122268091 | 133810703 | −0.3537 | −0.3704 | 1.0229 | 0.5243 | 1 | 1 |
| WCM78_Z1_1 | 13 | 19600371 | 30003017 | −0.1011 | 0.0351 | 1.0621 | 0.9871 | 1 | 1 |
| WCM78_Z1_1 | 13 | 30014128 | 33316780 | 0.1833 | 0.4492 | 1.569 | 1.1616 | 2 | 1 |
| WCM78_Z1_1 | 13 | 33327642 | 44456410 | −0.1313 | −0.0113 | 1.0427 | 0.9417 | 1 | 1 |
| WCM78_Z1_1 | 13 | 44457960 | 52293442 | −0.2997 | −0.28 | 1.0783 | 0.5689 | 1 | 1 |
| WCM78_Z1_1 | 13 | 52301897 | 109496798 | −0.1071 | 0.0259 | 1.0633 | 0.973 | 1 | 1 |
| WCM78_Z1_1 | 13 | 109507816 | 111927131 | 0.5386 | 0.9236 | NA | NA | NA | NA |
| WCM78_Z1_1 | 13 | 111927915 | 115089411 | −0.1751 | −0.0794 | 1.0266 | 0.8662 | 1 | 1 |
| WCM78_Z1_1 | 14 | 19377689 | 22005013 | −0.1485 | −0.0379 | 1.0104 | 0.9378 | 1 | 1 |
| WCM78_Z1_1 | 14 | 22038042 | 24429152 | 0.7697 | 1.214 | 3.6089 | 1.0307 | 4 | 1 |
| WCM78_Z1_1 | 14 | 24439304 | 31077205 | −0.1442 | −0.0311 | 1.0181 | 0.9392 | 1 | 1 |
| WCM78_Z1_1 | 14 | 31081399 | 34419856 | 0.2539 | 0.5466 | 1.9045 | 1.0168 | 2 | 1 |
| WCM78_Z1_1 | 14 | 34904416 | 36041904 | 0.8575 | 1.3215 | NA | NA | NA | NA |
| WCM78_Z1_1 | 14 | 36064978 | 37203747 | 0.3274 | 0.6464 | NA | NA | NA | NA |
| WCM78_Z1_1 | 14 | 37344183 | 72457513 | −0.1246 | −0.001 | 1.0316 | 0.967 | 1 | 1 |
| WCM78_Z1_1 | 14 | 72818848 | 75585587 | 0.2243 | 0.506 | 1.9509 | 0.8893 | 2 | 1 |
| WCM78_Z1_1 | 14 | 75587265 | 78744993 | −0.1656 | −0.0645 | 1.0205 | 0.892 | 1 | 1 |
| WCM78_Z1_1 | 14 | 78748484 | 88946216 | 0.2362 | 0.5224 | NA | NA | NA | NA |
| WCM78_Z1_1 | 14 | 88946504 | 91467472 | −0.1081 | 0.0243 | NA | NA | NA | NA |
| WCM78_Z1_1 | 14 | 91526748 | 92251540 | 0.4138 | 0.7612 | 2.3516 | 1.0383 | 2 | 1 |
| WCM78_Z1_1 | 14 | 92253956 | 107283183 | −0.1014 | 0.0346 | 1.043 | 1.0055 | 1 | 1 |
| WCM78_Z1_1 | 15 | 20170150 | 36937461 | −0.149 | −0.0386 | 1.2004 | 0.7468 | 1 | 1 |
| WCM78_Z1_1 | 15 | 36946303 | 42974689 | 0.0775 | 0.2994 | 1.3721 | 1.0891 | 1 | 1 |
| WCM78_Z1_1 | 15 | 42976351 | 52090435 | −0.1648 | −0.0632 | 1.0513 | 0.8629 | 1 | 1 |
| WCM78_Z1_1 | 15 | 52098590 | 56436621 | 0.2584 | 0.5528 | 1.856 | 1.0779 | 2 | 1 |
| WCM78_Z1_1 | 15 | 56471494 | 65147238 | −0.0577 | 0.1008 | 1.1143 | 1.0305 | 1 | 1 |
| WCM78_Z1_1 | 15 | 65152152 | 65739223 | 0.4315 | 0.7846 | NA | NA | NA | NA |
| WCM78_Z1_1 | 15 | 65743407 | 71704162 | −0.1702 | −0.0717 | 0.9873 | 0.9158 | 1 | 1 |
| WCM78_Z1_1 | 15 | 71839794 | 73409151 | 0.253 | 0.5454 | NA | NA | NA | NA |
| WCM78_Z1_1 | 15 | 73414888 | 75660826 | −0.1689 | −0.0697 | 0.9901 | 0.9156 | 1 | 1 |
| WCM78_Z1_1 | 15 | 75664417 | 77176198 | 0.4981 | 0.8714 | NA | NA | NA | NA |
| WCM78_Z1_1 | 15 | 77197684 | 79382831 | −0.1464 | −0.0346 | 1.005 | 0.9475 | 1 | 1 |
| WCM78_Z1_1 | 15 | 79502141 | 82575402 | 0.2637 | 0.56 | 1.8869 | 1.0616 | 2 | 1 |
| WCM78_Z1_1 | 15 | 83215194 | 86813174 | −0.0491 | 0.1136 | 1.1176 | 1.0463 | 1 | 1 |
| WCM78_Z1_1 | 15 | 86814912 | 93616975 | 0.2985 | 0.6074 | 1.9338 | 1.1132 | 2 | 1 |
| WCM78_Z1_1 | 15 | 93749491 | 102506276 | −0.1158 | 0.0126 | 1.066 | 0.9515 | 1 | 1 |
| WCM78_Z1_1 | 16 | 67604 | 836829 | −0.1665 | −0.0659 | 1.0121 | 0.8986 | 1 | 1 |
| WCM78_Z1_1 | 16 | 837073 | 2880797 | 0.2446 | 0.534 | 1.9326 | 0.9632 | 2 | 1 |
| WCM78_Z1_1 | 16 | 2881928 | 8722958 | −0.1754 | −0.0799 | 0.964 | 0.9283 | 1 | 1 |
| WCM78_Z1_1 | 16 | 8729124 | 11991733 | 0.1245 | 0.3665 | 1.3477 | 1.2307 | 1 | 1 |
| WCM78_Z1_1 | 16 | 12008872 | 33965562 | −0.1432 | −0.0297 | 1.0055 | 0.9538 | 1 | 1 |
| WCM78_Z1_1 | 16 | 34257013 | 48296699 | 0.1829 | 0.4485 | NA | NA | NA | NA |
| WCM78_Z1_1 | 16 | 48304001 | 48596287 | 0.0024 | 0.1901 | NA | NA | NA | NA |
| WCM78_Z1_1 | 16 | 48643720 | 54509664 | −0.3651 | −0.3899 | 1.1316 | 0.3948 | 1 | 0 |
| WCM78_Z1_1 | 16 | 54691223 | 84922841 | −0.0325 | 0.1385 | 1.1319 | 1.0696 | 1 | 1 |
| WCM78_Z1_1 | 16 | 84940224 | 85392987 | 0.0388 | 0.2434 | NA | NA | NA | NA |
| WCM78_Z1_1 | 16 | 85646998 | 88870460 | 0.3205 | 0.637 | 1.8775 | 1.2327 | 2 | 1 |
| WCM78_Z1_1 | 16 | 88871202 | 90233626 | 0.0372 | 0.241 | 1.2713 | 1.0924 | 1 | 1 |
| WCM78_Z1_1 | 17 | 131595 | 3794004 | −0.1293 | −0.0082 | 1.0541 | 0.9346 | 1 | 1 |
| WCM78_Z1_1 | 17 | 3801316 | 4012978 | −0.7687 | −1.1663 | 0.8825 | 0.0087 | 1 | 0 |
| WCM78_Z1_1 | 17 | 4015997 | 6703366 | −0.1452 | −0.0328 | 0.9927 | 0.9624 | 1 | 1 |
| WCM78_Z1_1 | 17 | 6704132 | 8065671 | −0.7254 | −1.0722 | 0.9968 | −0.0456 | 1 | 0 |
| WCM78_Z1_1 | 17 | 8077550 | 16747580 | −0.1427 | −0.0289 | 1.0075 | 0.9528 | 1 | 1 |
| WCM78_Z1_1 | 17 | 16836425 | 18003011 | −0.7078 | −1.0351 | NA | NA | NA | NA |
| WCM78_Z1_1 | 17 | 18003922 | 19480843 | −0.1854 | −0.0955 | 0.9875 | 0.8843 | 1 | 1 |
| WCM78_Z1_1 | 17 | 19534050 | 29705944 | 0.2306 | 0.5147 | 1.6962 | 1.1611 | 2 | 1 |
| WCM78_Z1_1 | 17 | 29708959 | 35656225 | −0.1528 | −0.0446 | 1.0359 | 0.9032 | 1 | 1 |
| WCM78_Z1_1 | 17 | 35687144 | 39346219 | 0.276 | 0.5768 | 2.0011 | 0.982 | 2 | 1 |
| WCM78_Z1_1 | 17 | 39346578 | 41165901 | 0.1306 | 0.3751 | 1.4089 | 1.185 | 1 | 1 |
| WCM78_Z1_1 | 17 | 41166265 | 53120662 | −0.1521 | −0.0434 | 1.002 | 0.9387 | 1 | 1 |
| WCM78_Z1_1 | 17 | 53150347 | 55607038 | 0.278 | 0.5795 | NA | NA | NA | NA |
| WCM78_Z1_1 | 17 | 55693375 | 57643094 | −0.1277 | −0.0057 | 1.1019 | 0.8902 | 1 | 1 |
| WCM78_Z1_1 | 17 | 57644016 | 58767060 | 0.2274 | 0.5103 | NA | NA | NA | NA |
| WCM78_Z1_1 | 17 | 58786645 | 73043678 | −0.0979 | 0.0399 | 1.061 | 0.9952 | 1 | 1 |
| WCM78_Z1_1 | 17 | 73045419 | 73780688 | 0.4408 | 0.7967 | 2.5541 | 0.9202 | 3 | 1 |
| WCM78_Z1_1 | 17 | 73780948 | 79010722 | −0.091 | 0.0505 | 1.0939 | 0.9774 | 1 | 1 |
| WCM78_Z1_1 | 17 | 79027491 | 81083567 | 0.2186 | 0.4981 | 1.8337 | 0.991 | 2 | 1 |
| WCM78_Z1_1 | 18 | 48213 | 24039644 | −0.0596 | 0.0979 | 1.2311 | 0.9094 | 1 | 1 |
| WCM78_Z1_1 | 18 | 24039842 | 32677541 | 0.2813 | 0.584 | 1.9513 | 1.0467 | 2 | 1 |
| WCM78_Z1_1 | 18 | 32682002 | 78005215 | −0.1224 | 0.0024 | 1.0356 | 0.9678 | 1 | 1 |
| WCM78_Z1_1 | 19 | 281506 | 1863095 | −0.409 | −0.4655 | 0.9831 | 0.4654 | 1 | 0 |
| WCM78_Z1_1 | 19 | 1877406 | 3381857 | −0.1563 | −0.0501 | 1.2262 | 0.7056 | 1 | 1 |
| WCM78_Z1_1 | 19 | 3382096 | 5206820 | 0.3264 | 0.645 | 1.6667 | 1.4607 | 2 | 1 |
| WCM78_Z1_1 | 19 | 5207999 | 17531833 | −0.1226 | 0.0022 | 1.0832 | 0.9199 | 1 | 1 |
| WCM78_Z1_1 | 19 | 17533221 | 20117187 | 0.2363 | 0.5225 | 1.867 | 1.0059 | 2 | 1 |
| WCM78_Z1_1 | 19 | 20117424 | 36593955 | −0.103 | 0.0322 | 1.1584 | 0.8868 | 1 | 1 |
| WCM78_Z1_1 | 19 | 36594463 | 40009567 | 0.0307 | 0.2316 | 1.4258 | 0.9224 | 1 | 1 |
| WCM78_Z1_1 | 19 | 40023002 | 48389415 | −0.1509 | −0.0416 | 0.9988 | 0.9444 | 1 | 1 |
| WCM78_Z1_1 | 19 | 48480032 | 51363383 | 0.2498 | 0.541 | 1.9334 | 0.9765 | 2 | 1 |
| WCM78_Z1_1 | 19 | 51376759 | 51742921 | 0.6238 | 1.0321 | 3.2386 | 0.8515 | 3 | 1 |
| WCM78_Z1_1 | 19 | 51767330 | 53086668 | 0.3277 | 0.6468 | 2.1716 | 0.9597 | 2 | 1 |
| WCM78_Z1_1 | 19 | 53100220 | 59083929 | −0.1444 | −0.0315 | 1.0011 | 0.9556 | 1 | 1 |
| WCM78_Z1_1 | 2 | 41615 | 6980746 | −0.1461 | −0.0341 | 1.0357 | 0.9175 | 1 | 1 |
| WCM78_Z1_1 | 2 | 6990102 | 10350703 | 0.2749 | 0.5754 | 1.8937 | 1.0864 | 2 | 1 |
| WCM78_Z1_1 | 2 | 10559979 | 60614577 | −0.0941 | 0.0457 | 1.0621 | 1.0024 | 1 | 1 |
| WCM78_Z1_1 | 2 | 60679791 | 65488378 | 0.3425 | 0.6666 | 2.1698 | 1.0049 | 2 | 1 |
| WCM78_Z1_1 | 2 | 65492355 | 68805107 | −0.2031 | −0.1235 | NA | NA | NA | NA |
| WCM78_Z1_1 | 2 | 68873075 | 70165237 | 0.2508 | 0.5424 | NA | NA | NA | NA |
| WCM78_Z1_1 | 2 | 70187808 | 80874871 | −0.1505 | −0.041 | 1.0082 | 0.9357 | 1 | 1 |
| WCM78_Z1_1 | 2 | 84518074 | 86255758 | 0.5478 | 0.9354 | 2.0104 | 1.8144 | 2 | 2 |
| WCM78_Z1_1 | 2 | 86257383 | 100343553 | −0.1477 | −0.0367 | 1.0581 | 0.8917 | 1 | 1 |
| WCM78_Z1_1 | 2 | 100368724 | 100722390 | 0.2329 | 0.5178 | NA | NA | NA | NA |
| WCM78_Z1_1 | 2 | 100900841 | 102803488 | 0.4099 | 0.7561 | NA | NA | NA | NA |
| WCM78_Z1_1 | 2 | 102804374 | 122139867 | 0.0312 | 0.2324 | 1.4472 | 0.9024 | 1 | 1 |
| WCM78_Z1_1 | 2 | 122144729 | 125521274 | −0.1247 | −0.0011 | NA | NA | NA | NA |
| WCM78_Z1_1 | 2 | 125521617 | 157439316 | −0.4159 | −0.4775 | 0.9381 | 0.4983 | 1 | 0 |
| WCM78_Z1_1 | 2 | 158114632 | 178080580 | −0.2554 | −0.2074 | 1.3071 | 0.4251 | 1 | 0 |
| WCM78_Z1_1 | 2 | 178081314 | 179593862 | −0.4061 | −0.4605 | 0.9912 | 0.4623 | 1 | 0 |
| WCM78_Z1_1 | 2 | 179594021 | 180851439 | 0.0575 | 0.2705 | NA | NA | NA | NA |
| WCM78_Z1_1 | 2 | 180853362 | 189342436 | −0.3721 | −0.4018 | 1.0471 | 0.4667 | 1 | 0 |
| WCM78_Z1_1 | 2 | 189348178 | 196682553 | −0.2217 | −0.1533 | 0.9941 | 0.8043 | 1 | 1 |
| WCM78_Z1_1 | 2 | 196688986 | 204732752 | 0.3439 | 0.6684 | 1.7894 | 1.3893 | 2 | 1 |
| WCM78_Z1_1 | 2 | 204735439 | 212293145 | −0.1809 | −0.0885 | 0.9553 | 0.9257 | 1 | 1 |
| WCM78_Z1_1 | 2 | 212295777 | 218683154 | 0.2274 | 0.5102 | NA | NA | NA | NA |
| WCM78_Z1_1 | 2 | 218683278 | 234969120 | −0.1869 | −0.098 | 0.9623 | 0.9064 | 1 | 1 |
| WCM78_Z1_1 | 2 | 234975187 | 242946616 | −0.4722 | −0.5776 | 0.9033 | 0.4368 | 1 | 0 |
| WCM78_Z1_1 | 20 | 68406 | 4893538 | −0.3842 | −0.4225 | 0.9992 | 0.4931 | 1 | 0 |
| WCM78_Z1_1 | 20 | 4913140 | 6759696 | 0.0376 | 0.2416 | 1.9687 | 0.3959 | 2 | 0 |
| WCM78_Z1_1 | 20 | 7864284 | 19664881 | −0.2861 | −0.2576 | 1.1046 | 0.5684 | 1 | 1 |
| WCM78_Z1_1 | 20 | 19665779 | 62919090 | −0.1211 | 0.0044 | 1.0277 | 0.9784 | 1 | 1 |
| WCM78_Z1_1 | 21 | 9907291 | 36421166 | −0.0881 | 0.0549 | 1.0845 | 0.993 | 1 | 1 |
| WCM78_Z1_1 | 21 | 37093097 | 39087397 | 0.2724 | 0.572 | 2.0252 | 0.9479 | 2 | 1 |
| WCM78_Z1_1 | 21 | 39212977 | 48084277 | −0.1458 | −0.0337 | 1.0148 | 0.9391 | 1 | 1 |
| WCM78_Z1_1 | 22 | 16287437 | 51215153 | −0.1542 | −0.0467 | 0.9847 | 0.9516 | 1 | 1 |
| WCM78_Z1_1 | 3 | 239347 | 12660190 | −0.1316 | −0.0118 | 1.0087 | 0.9751 | 1 | 1 |
| WCM78_Z1_1 | 3 | 12776286 | 15292465 | 0.2439 | 0.5329 | 1.967 | 0.9267 | 2 | 1 |
| WCM78_Z1_1 | 3 | 15297803 | 44827925 | −0.0728 | 0.0781 | 1.1178 | 0.9935 | 1 | 1 |
| WCM78_Z1_1 | 3 | 44835729 | 48485278 | 0.2463 | 0.5362 | 1.8539 | 1.0465 | 2 | 1 |
| WCM78_Z1_1 | 3 | 48488362 | 65344822 | −0.1283 | −0.0067 | 1.0061 | 0.9847 | 1 | 1 |
| WCM78_Z1_1 | 3 | 65346941 | 69434992 | 0.8135 | 1.2678 | NA | NA | NA | NA |
| WCM78_Z1_1 | 3 | 69788852 | 107097239 | −0.1452 | −0.0328 | 1.0321 | 0.923 | 1 | 1 |
| WCM78_Z1_1 | 3 | 107378588 | 109136690 | 0.2345 | 0.52 | NA | NA | NA | NA |
| WCM78_Z1_1 | 3 | 109178730 | 121822663 | −0.1191 | 0.0075 | 1.0562 | 0.9543 | 1 | 1 |
| WCM78_Z1_1 | 3 | 121825197 | 124397114 | 0.2715 | 0.5707 | 1.9609 | 1.0097 | 2 | 1 |
| WCM78_Z1_1 | 3 | 124398358 | 127786882 | −0.1539 | −0.0463 | 1.0016 | 0.9353 | 1 | 1 |
| WCM78_Z1_1 | 3 | 127788409 | 129290379 | 0.2099 | 0.486 | NA | NA | NA | NA |
| WCM78_Z1_1 | 3 | 129290616 | 160037645 | −0.1052 | 0.0288 | 1.0357 | 1.0046 | 1 | 1 |
| WCM78_Z1_1 | 3 | 160061568 | 167167191 | 0.1981 | 0.4697 | NA | NA | NA | NA |
| WCM78_Z1_1 | 3 | 167183176 | 186504569 | −0.0882 | 0.0548 | 1.0545 | 1.0229 | 1 | 1 |
| WCM78_Z1_1 | 3 | 186505104 | 189604187 | 0.4126 | 0.7597 | 1.7333 | 1.653 | 2 | 2 |
| WCM78_Z1_1 | 3 | 189607204 | 197894712 | −0.1431 | −0.0294 | 1.0117 | 0.9479 | 1 | 1 |
| WCM78_Z1_1 | 4 | 59331 | 533001 | −0.1581 | −0.0528 | NA | NA | NA | NA |
| WCM78_Z1_1 | 4 | 619657 | 1993416 | 0.0844 | 0.3092 | 1.4435 | 1.0346 | 1 | 1 |
| WCM78_Z1_1 | 4 | 1994151 | 39233465 | −0.145 | −0.0325 | 1.0172 | 0.9383 | 1 | 1 |
| WCM78_Z1_1 | 4 | 39233790 | 52740525 | 0.2499 | 0.5412 | 1.9595 | 0.9508 | 2 | 1 |
| WCM78_Z1_1 | 4 | 52743961 | 71066297 | −0.1245 | −8.00E−04 | 1.0256 | 0.9733 | 1 | 1 |
| WCM78_Z1_1 | 4 | 71067194 | 75971400 | 0.3899 | 0.7298 | 2.2669 | 1.0499 | 2 | 1 |
| WCM78_Z1_1 | 4 | 76407786 | 86643660 | −0.1625 | −0.0596 | 0.979 | 0.9401 | 1 | 1 |
| WCM78_Z1_1 | 4 | 86844835 | 88903881 | 0.2766 | 0.5777 | 1.9961 | 0.9888 | 2 | 1 |
| WCM78_Z1_1 | 4 | 88903986 | 174537676 | −0.1356 | −0.0178 | 1.0259 | 0.9496 | 1 | 1 |
| WCM78_Z1_1 | 4 | 175158604 | 186112111 | 0.1187 | 0.3583 | 1.5823 | 0.9815 | 2 | 1 |
| WCM78_Z1_1 | 4 | 186168530 | 190426502 | −0.1203 | 0.0057 | 1.0857 | 0.9222 | 1 | 1 |
| WCM78_Z1_1 | 5 | 140666 | 3596449 | 0.2342 | 0.5196 | 1.9265 | 0.9405 | 2 | 1 |
| WCM78_Z1_1 | 5 | 3599378 | 14474161 | −0.1469 | −0.0353 | 1.0167 | 0.9349 | 1 | 1 |
| WCM78_Z1_1 | 5 | 14477015 | 16935914 | 0.5216 | 0.9018 | NA | NA | NA | NA |
| WCM78_Z1_1 | 5 | 17275470 | 35089682 | −0.1443 | −0.0313 | 1.0438 | 0.9133 | 1 | 1 |
| WCM78_Z1_1 | 5 | 35618132 | 37479988 | 0.6381 | 1.0501 | NA | NA | NA | NA |
| WCM78_Z1_1 | 5 | 37605267 | 53467637 | −0.1701 | −0.0715 | 1.0157 | 0.8876 | 1 | 1 |
| WCM78_Z1_1 | 5 | 53606295 | 56111876 | 0.3023 | 0.6124 | 2.1759 | 0.8818 | 2 | 1 |
| WCM78_Z1_1 | 5 | 56152496 | 136602727 | −0.1039 | 0.0308 | 1.0622 | 0.981 | 1 | 1 |
| WCM78_Z1_1 | 5 | 136834178 | 138287542 | 0.2591 | 0.5537 | NA | NA | NA | NA |
| WCM78_Z1_1 | 5 | 138356873 | 154397093 | −0.1128 | 0.0172 | 1.0337 | 0.9903 | 1 | 1 |
| WCM78_Z1_1 | 5 | 155756587 | 159841437 | 0.3151 | 0.6298 | 2.0938 | 1.0009 | 2 | 1 |
| WCM78_Z1_1 | 5 | 159842257 | 169064362 | −0.1312 | −0.0111 | 1.0153 | 0.9694 | 1 | 1 |
| WCM78_Z1_1 | 5 | 169081425 | 172384183 | 0.2765 | 0.5775 | 2.0101 | 0.9744 | 2 | 1 |
| WCM78_Z1_1 | 5 | 172385401 | 180687773 | −0.1869 | −0.098 | 0.9638 | 0.9049 | 1 | 1 |
| WCM78_Z1_1 | 6 | 203515 | 656915 | −0.1759 | −0.0807 | NA | NA | NA | NA |
| WCM78_Z1_1 | 6 | 707202 | 4131045 | 0.2669 | 0.5644 | 1.8842 | 1.0733 | 2 | 1 |
| WCM78_Z1_1 | 6 | 4133890 | 27775611 | −0.1283 | −0.0066 | 1.0276 | 0.9632 | 1 | 1 |
| WCM78_Z1_1 | 6 | 27775992 | 32007849 | 0.509 | 0.8855 | 2.7493 | 0.9455 | 3 | 1 |
| WCM78_Z1_1 | 6 | 32008543 | 33553460 | −0.073 | 0.0777 | NA | NA | NA | NA |
| WCM78_Z1_1 | 6 | 33554532 | 35715129 | 0.4287 | 0.7809 | NA | NA | NA | NA |
| WCM78_Z1_1 | 6 | 35715358 | 41774635 | −0.0404 | 0.1267 | 1.1619 | 1.0217 | 1 | 1 |
| WCM78_Z1_1 | 6 | 41874919 | 44084286 | 0.3273 | 0.6462 | 1.931 | 1.199 | 2 | 1 |
| WCM78_Z1_1 | 6 | 44102327 | 51547037 | −0.155 | −0.0479 | 1.1149 | 0.8198 | 1 | 1 |
| WCM78_Z1_1 | 6 | 51586814 | 65622528 | 0.109 | 0.3444 | 1.5662 | 0.9731 | 2 | 1 |
| WCM78_Z1_1 | 6 | 65655779 | 107040160 | −0.131 | −0.0107 | 1.0462 | 0.939 | 1 | 1 |
| WCM78_Z1_1 | 6 | 107050770 | 119623239 | 0.2427 | 0.5313 | 1.834 | 1.0564 | 2 | 1 |
| WCM78_Z1_1 | 6 | 119628098 | 128294933 | −0.1456 | −0.0334 | 1.0423 | 0.9119 | 1 | 1 |
| WCM78_Z1_1 | 6 | 128297866 | 130762903 | 0.4837 | 0.8527 | NA | NA | NA | NA |
| WCM78_Z1_1 | 6 | 131148737 | 137815297 | −0.1412 | −0.0266 | 1.0015 | 0.962 | 1 | 1 |
| WCM78_Z1_1 | 6 | 138192536 | 147704045 | 0.1013 | 0.3335 | 1.4345 | 1.0856 | 1 | 1 |
| WCM78_Z1_1 | 6 | 147830073 | 166952263 | −0.1378 | −0.0213 | 1.0021 | 0.9686 | 1 | 1 |
| WCM78_Z1_1 | 6 | 167040508 | 170893409 | −0.679 | −0.975 | 0.9842 | 0.0333 | 1 | 0 |
| WCM78_Z1_1 | 7 | 193451 | 3188637 | 0.5618 | 0.9533 | 2.8123 | 1.0602 | 3 | 1 |
| WCM78_Z1_1 | 7 | 3197867 | 30029844 | 0.2568 | 0.5506 | 1.9494 | 0.9799 | 2 | 1 |
| WCM78_Z1_1 | 7 | 30054475 | 33384197 | 0.8429 | 1.3038 | NA | NA | NA | NA |
| WCM78_Z1_1 | 7 | 33385839 | 49815105 | 0.0648 | 0.2811 | 1.4817 | 0.9486 | 1 | 1 |
| WCM78_Z1_1 | 7 | 49815390 | 65439658 | −0.1703 | −0.0719 | 1.0005 | 0.9022 | 1 | 1 |
| WCM78_Z1_1 | 7 | 65440053 | 79833065 | 0.1841 | 0.4503 | 1.7758 | 0.9568 | 2 | 1 |
| WCM78_Z1_1 | 7 | 79840350 | 94043586 | −0.1707 | −0.0725 | 1.0225 | 0.8795 | 1 | 1 |
| WCM78_Z1_1 | 7 | 94044543 | 100839424 | 0.0296 | 0.23 | 1.4142 | 0.9316 | 1 | 1 |
| WCM78_Z1_1 | 7 | 100839496 | 103848322 | 0.2322 | 0.5169 | 1.7479 | 1.1138 | 2 | 1 |
| WCM78_Z1_1 | 7 | 103969576 | 123143356 | −0.065 | 0.0897 | 1.194 | 0.9344 | 1 | 1 |
| WCM78_Z1_1 | 7 | 123152035 | 135421928 | 0.2466 | 0.5366 | 1.8166 | 1.0846 | 2 | 1 |
| WCM78_Z1_1 | 7 | 135433327 | 147092792 | −0.1455 | −0.0333 | 0.9878 | 0.9666 | 1 | 1 |
| WCM78_Z1_1 | 7 | 147183104 | 148724945 | 0.4585 | 0.8199 | NA | NA | NA | NA |
| WCM78_Z1_1 | 7 | 148725458 | 150028027 | 0.7052 | 1.1342 | 2.3316 | 2.0582 | 2 | 2 |
| WCM78_Z1_1 | 7 | 150034173 | 151970797 | 0.4513 | 0.8105 | 2.6409 | 0.8667 | 3 | 1 |
| WCM78_Z1_1 | 7 | 152007133 | 158937066 | −0.6469 | −0.9099 | 1.0879 | −0.0235 | 1 | 0 |
| WCM78_Z1_1 | 8 | 190908 | 4277512 | −0.1567 | −0.0506 | 0.9986 | 0.9324 | 1 | 1 |
| WCM78_Z1_1 | 8 | 4494996 | 8749985 | 0.3125 | 0.6262 | 1.8954 | 1.1917 | 2 | 1 |
| WCM78_Z1_1 | 8 | 8750407 | 54155419 | −0.1174 | 0.0102 | 1.038 | 0.9762 | 1 | 1 |
| WCM78_Z1_1 | 8 | 54163541 | 69017590 | 0.0742 | 0.2946 | 1.373 | 1.08 | 1 | 1 |
| WCM78_Z1_1 | 8 | 69018574 | 76476328 | 0.397 | 0.7392 | 2.1483 | 1.1901 | 2 | 1 |
| WCM78_Z1_1 | 8 | 77616345 | 100654723 | 0.0924 | 0.3208 | 1.3062 | 1.1918 | 1 | 1 |
| WCM78_Z1_1 | 8 | 100711890 | 103663925 | 0.8344 | 1.2934 | NA | NA | NA | NA |
| WCM78_Z1_1 | 8 | 103664206 | 122626403 | 0.0972 | 0.3275 | 1.4468 | 1.063 | 1 | 1 |
| WCM78_Z1_1 | 8 | 122626551 | 132998459 | 0.4564 | 0.8172 | 2.5464 | 0.9775 | 3 | 1 |
| WCM78_Z1_1 | 8 | 132999908 | 134225367 | 0.7034 | 1.1319 | NA | NA | NA | NA |
| WCM78_Z1_1 | 8 | 134232996 | 136657349 | 1.7178 | 2.3151 | NA | NA | NA | NA |
| WCM78_Z1_1 | 8 | 139144868 | 144377983 | 0.7408 | 1.1784 | 3.6501 | 0.8764 | 4 | 1 |
| WCM78_Z1_1 | 8 | 144378283 | 145806310 | 0.9762 | 1.4645 | 3.9272 | 1.5921 | 4 | 2 |
| WCM78_Z1_1 | 8 | 145806491 | 146279472 | 0.7062 | 1.1354 | 3.4414 | 0.9523 | 3 | 1 |
| WCM78_Z1_1 | 9 | 17060 | 2039814 | −0.1805 | −0.0878 | 1.0652 | 0.8167 | 1 | 1 |
| WCM78_Z1_1 | 9 | 2047452 | 5535038 | 0.2434 | 0.5322 | NA | NA | NA | NA |
| WCM78_Z1_1 | 9 | 5549588 | 12709175 | −0.1079 | 0.0246 | NA | NA | NA | NA |
| WCM78_Z1_1 | 9 | 12775909 | 16253063 | 0.2996 | 0.6088 | NA | NA | NA | NA |
| WCM78_Z1_1 | 9 | 16419334 | 32784197 | −0.1317 | −0.0119 | 1.0163 | 0.9673 | 1 | 1 |
| WCM78_Z1_1 | 9 | 32784640 | 35107517 | 0.2383 | 0.5253 | 1.8446 | 1.0338 | 2 | 1 |
| WCM78_Z1_1 | 9 | 35107765 | 36276966 | −0.0884 | 0.0544 | 1.0526 | 1.0242 | 1 | 1 |
| WCM78_Z1_1 | 9 | 36339787 | 38622116 | 0.2541 | 0.5469 | NA | NA | NA | NA |
| WCM78_Z1_1 | 9 | 39078450 | 77277434 | −0.1594 | −0.0549 | 1.0152 | 0.9101 | 1 | 1 |
| WCM78_Z1_1 | 9 | 77280458 | 86281347 | 0.154 | 0.4081 | 1.4706 | 1.1833 | 1 | 1 |
| WCM78_Z1_1 | 9 | 86292711 | 100698451 | −0.1462 | −0.0343 | 1.0144 | 0.9386 | 1 | 1 |
| WCM78_Z1_1 | 9 | 100700351 | 104433034 | 0.2026 | 0.476 | 1.5259 | 1.2558 | 2 | 1 |
| WCM78_Z1_1 | 9 | 104433235 | 141111414 | −0.153 | −0.0449 | 0.9829 | 0.9558 | 1 | 1 |
| WCM78_Z1_1 | X | 2707724 | 11130205 | −0.1616 | −0.0582 | NA | NA | NA | NA |
| WCM78_Z1_1 | X | 11136454 | 27999373 | 0.7075 | 1.1371 | NA | NA | NA | NA |
| WCM78_Z1_1 | X | 28807515 | 41206927 | −0.2364 | −0.1767 | NA | NA | NA | NA |
| WCM78_Z1_1 | X | 41307160 | 48435908 | 0.4223 | 0.7724 | NA | NA | NA | NA |
| WCM78_Z1_1 | X | 48542706 | 65486378 | 0.0827 | 0.3068 | NA | NA | NA | NA |
| WCM78_Z1_1 | X | 65819524 | 67944205 | 1.6731 | 2.2654 | NA | NA | NA | NA |
| WCM78_Z1_1 | X | 68049652 | 127186187 | −0.2133 | −0.1397 | NA | NA | NA | NA |
| WCM78_Z1_1 | X | 128582347 | 134305059 | 0.1188 | 0.3584 | NA | NA | NA | NA |
| WCM78_Z1_1 | X | 134655007 | 154509348 | −0.1786 | −0.0849 | NA | NA | NA | NA |
| WCM7_Tissue | 1 | 14716 | 16254698 | −0.6288 | −0.9144 | 1.049 | 0.0121 | 1 | 0 |
| WCM7_Tissue | 1 | 16255132 | 17087465 | −0.5107 | −0.7372 | 0.8786 | 0.3212 | 1 | 0 |
| WCM7_Tissue | 1 | 17248521 | 37967613 | −0.646 | −0.9408 | 1.0614 | −0.0195 | 1 | 0 |
| WCM7_Tissue | 1 | 37975029 | 39318120 | −0.0412 | −0.0906 | 1.0759 | 0.8024 | 1 | 1 |
| WCM7_Tissue | 1 | 39321475 | 144679970 | −0.5849 | −0.8476 | 1.0969 | 0.0145 | 1 | 0 |
| WCM7_Tissue | 1 | 144811819 | 249212558 | 0.0546 | 0.0327 | 1.1234 | 0.9224 | 1 | 1 |
| WCM7_Tissue | 10 | 226016 | 39006437 | −0.0017 | −0.0394 | 0.9956 | 0.9504 | 1 | 1 |
| WCM7_Tissue | 10 | 39011230 | 44866798 | −0.5584 | −0.808 | NA | NA | NA | NA |
| WCM7_Tissue | 10 | 44868828 | 54531331 | 0.0427 | 0.0176 | 1.2388 | 0.7858 | 1 | 1 |
| WCM7_Tissue | 10 | 55566646 | 96602719 | −0.6222 | −0.9044 | 1.0494 | 0.0192 | 1 | 0 |
| WCM7_Tissue | 10 | 96609769 | 135477937 | 0.0793 | 0.0642 | 1.0946 | 0.9964 | 1 | 1 |
| WCM7_Tissue | 11 | 193096 | 134257494 | 0.0516 | 0.029 | 1.1453 | 0.8953 | 1 | 1 |
| WCM7_Tissue | 12 | 247582 | 109042588 | 0.0636 | 0.0442 | 1.0469 | 1.0154 | 1 | 1 |
| WCM7_Tissue | 12 | 109046120 | 123201311 | −0.1332 | −0.2114 | 0.882 | 0.8454 | 1 | 1 |
| WCM7_Tissue | 12 | 123213956 | 133779351 | 3.00E−04 | −0.0369 | 0.9999 | 0.9496 | 1 | 1 |
| WCM7_Tissue | 13 | 19240930 | 115089411 | −0.6332 | −0.9212 | 1.0514 | 0.0047 | 1 | 0 |
| WCM7_Tissue | 14 | 19377775 | 107283088 | 0.0603 | 0.04 | 1.0713 | 0.9849 | 1 | 1 |
| WCM7_Tissue | 15 | 20169986 | 22709666 | −0.2867 | −0.4188 | 1.0808 | 0.4153 | 1 | 0 |
| WCM7_Tissue | 15 | 22782337 | 102516473 | −0.6345 | −0.9231 | 1.0495 | 0.0053 | 1 | 0 |
| WCM7_Tissue | 16 | 64456 | 50136324 | −0.0246 | −0.069 | 1.062 | 0.8446 | 1 | 1 |
| WCM7_Tissue | 16 | 50245309 | 90162517 | −0.6791 | −0.9922 | 1.0285 | −0.0231 | 1 | 0 |
| WCM7_Tissue | 17 | 131595 | 81188087 | 0.0285 | −7.00E−04 | 1.0969 | 0.9021 | 1 | 1 |
| WCM7_Tissue | 18 | 47725 | 24056669 | 0.0552 | 0.0336 | 1.1489 | 0.8982 | 1 | 1 |
| WCM7_Tissue | 18 | 24081044 | 74154807 | −0.6652 | −0.9706 | 1.0214 | −8.00E−04 | 1 | 0 |
| WCM7_Tissue | 18 | 74536282 | 78005194 | 0.0572 | 0.036 | 1.0841 | 0.9665 | 1 | 1 |
| WCM7_Tissue | 19 | 110724 | 2901016 | −0.0152 | −0.0569 | 1.1006 | 0.8221 | 1 | 1 |
| WCM7_Tissue | 19 | 2915494 | 19627096 | −0.1373 | −0.2169 | 0.8958 | 0.825 | 1 | 1 |
| WCM7_Tissue | 19 | 19637074 | 59082684 | −0.0366 | −0.0847 | 1.0373 | 0.8486 | 1 | 1 |
| WCM7_Tissue | 2 | 41609 | 66795846 | 0.0845 | 0.0707 | 2.1214 | −0.0209 | 2 | 0 |
| WCM7_Tissue | 2 | 66796154 | 69769730 | 0.5967 | 0.6943 | NA | NA | NA | NA |
| WCM7_Tissue | 2 | 69771644 | 176856991 | 0.082 | 0.0676 | 2.1041 | −0.0081 | 2 | 0 |
| WCM7_Tissue | 2 | 176860268 | 179621460 | 0.3921 | 0.4509 | NA | NA | NA | NA |
| WCM7_Tissue | 2 | 179622318 | 243037091 | 0.078 | 0.0626 | 2.0988 | −0.0101 | 2 | 0 |
| WCM7_Tissue | 20 | 68415 | 62958909 | 0.0011 | −0.0359 | 1.0245 | 0.9264 | 1 | 1 |
| WCM7_Tissue | 21 | 9590334 | 48084177 | 0.0512 | 0.0284 | 1.0407 | 0.999 | 1 | 1 |
| WCM7_Tissue | 22 | 16255891 | 51216417 | −0.6664 | −0.9724 | 1.0195 | −2.00E−04 | 1 | 0 |
| WCM7_Tissue | 3 | 361470 | 132051063 | 0.0709 | 0.0534 | 1.0589 | 1.0166 | 1 | 1 |
| WCM7_Tissue | 3 | 132056349 | 197847562 | 0.4994 | 0.5794 | 1.9526 | 1.036 | 2 | 1 |
| WCM7_Tissue | 4 | 53403 | 40440902 | −0.6109 | −0.887 | 1.0714 | 0.01 | 1 | 0 |
| WCM7_Tissue | 4 | 40752866 | 164466928 | 0.0418 | 0.0164 | 1.027 | 0.9959 | 1 | 1 |
| WCM7_Tissue | 4 | 164507052 | 177650817 | −0.5864 | −0.8499 | 1.1406 | −0.031 | 1 | 0 |
| WCM7_Tissue | 4 | 177713345 | 190947521 | 0.0935 | 0.0822 | 1.0841 | 1.0332 | 1 | 1 |
| WCM7_Tissue | 5 | 140532 | 52398000 | 0.5392 | 0.6266 | 1.9968 | 1.0911 | 2 | 1 |
| WCM7_Tissue | 5 | 52402932 | 132073106 | 0.0952 | 0.0843 | 2.1367 | −0.0163 | 2 | 0 |
| WCM7_Tissue | 5 | 132084132 | 180687821 | 0.5096 | 0.5915 | 1.9415 | 1.0721 | 2 | 1 |
| WCM7_Tissue | 6 | 292536 | 64423298 | 0.0927 | 0.0812 | 1.1637 | 0.952 | 1 | 1 |
| WCM7_Tissue | 6 | 66044947 | 170892772 | −0.648 | −0.9439 | 1.0322 | 0.0074 | 1 | 0 |
| WCM7_Tissue | 7 | 195638 | 158935160 | 0.4968 | 0.5763 | 1.9183 | 1.0637 | 2 | 1 |
| WCM7_Tissue | 8 | 163536 | 36644900 | −0.6564 | −0.9569 | 1.0629 | −0.0325 | 1 | 0 |
| WCM7_Tissue | 8 | 36661538 | 48114334 | −0.0072 | −0.0466 | 1.0395 | 0.897 | 1 | 1 |
| WCM7_Tissue | 8 | 48508535 | 128972936 | 0.5001 | 0.5802 | 1.9678 | 1.0224 | 2 | 1 |
| WCM7_Tissue | 8 | 129021111 | 146279513 | 0.0921 | 0.0804 | 1.1913 | 0.9234 | 1 | 1 |
| WCM7_Tissue | 9 | 14885 | 15447280 | 0.0195 | −0.0122 | 1.0094 | 0.9737 | 1 | 1 |
| WCM7_Tissue | 9 | 15453096 | 28888864 | −0.7082 | −1.0378 | 1.0232 | −0.049 | 1 | 0 |
| WCM7_Tissue | 9 | 29999669 | 141071668 | 0.0579 | 0.037 | 1.1385 | 0.9135 | 1 | 1 |
| WCM7_Tissue | X | 2700146 | 154493585 | −0.0194 | −0.0624 | NA | NA | NA | NA |
| WCM80 | 1 | 14907 | 9995562 | 0.0513 | 0.0268 | 1.0245 | 1.013 | 1 | 1 |
| WCM80 | 1 | 9996718 | 11189787 | −0.4499 | −1.1218 | 0.9119 | 0.0072 | 1 | 0 |
| WCM80 | 1 | 11190646 | 226175904 | 0.0436 | 0.0124 | 1.0087 | 1.0087 | 1 | 1 |
| WCM80 | 1 | 226176043 | 227954677 | −0.4296 | −1.0632 | 0.9346 | 0.0225 | 1 | 0 |
| WCM80 | 1 | 227958225 | 249214018 | 0.041 | 0.0076 | 1.0053 | 1.0053 | 1 | 1 |
| WCM80 | 10 | 225938 | 135439027 | 0.0273 | −0.0184 | 0.9873 | 0.9873 | 1 | 1 |
| WCM80 | 11 | 193096 | 33881135 | 0.0865 | 0.0919 | 1.0658 | 1.0658 | 1 | 1 |
| WCM80 | 11 | 33886338 | 48347429 | −0.4206 | −1.0374 | 0.8087 | 0.1657 | 1 | 0 |
| WCM80 | 11 | 48366958 | 124135655 | 0.0336 | −0.0065 | 0.9955 | 0.9955 | 1 | 1 |
| WCM80 | 11 | 124179835 | 125366605 | −0.3855 | −0.9405 | 1.0602 | −0.0182 | 1 | 0 |
| WCM80 | 11 | 125442373 | 134605906 | 0.0379 | 0.0017 | 1.0012 | 1.0012 | 1 | 1 |
| WCM80 | 12 | 148388 | 104160293 | 0.0258 | −0.0213 | 0.9854 | 0.9854 | 1 | 1 |
| WCM80 | 12 | 104167656 | 108418514 | −0.4349 | −1.0782 | 0.8505 | 0.0968 | 1 | 0 |
| WCM80 | 12 | 108589691 | 110945998 | 0.0316 | −0.0103 | 0.9929 | 0.9929 | 1 | 1 |
| WCM80 | 12 | 110950705 | 112159518 | −0.4313 | −1.0678 | NA | NA | NA | NA |
| WCM80 | 12 | 112165777 | 124172706 | 9.00E−04 | −0.0688 | 0.9534 | 0.9534 | 1 | 1 |
| WCM80 | 12 | 124175056 | 125591689 | −0.461 | −1.1545 | 0.9235 | −0.0251 | 1 | 0 |
| WCM80 | 12 | 125591849 | 133810703 | 0.0656 | 0.0535 | 1.0378 | 1.0378 | 1 | 1 |
| WCM80 | 13 | 19041994 | 33020568 | −0.0058 | −0.0819 | 0.9448 | 0.9448 | 1 | 1 |
| WCM80 | 13 | 33054686 | 76111751 | −0.4875 | −1.2349 | 0.8315 | 0.0183 | 1 | 0 |
| WCM80 | 13 | 76124003 | 115089411 | −0.0217 | −0.1128 | 0.9339 | 0.9158 | 1 | 1 |
| WCM80 | 14 | 19110297 | 107283263 | 0.0262 | −0.0204 | 0.9859 | 0.9859 | 1 | 1 |
| WCM80 | 15 | 20083928 | 27017924 | 0.4217 | 0.6569 | 2.1112 | 1.0421 | 2 | 1 |
| WCM80 | 15 | 27018841 | 45879748 | −0.4707 | −1.1837 | 0.8888 | −0.0083 | 1 | 0 |
| WCM80 | 15 | 45884450 | 102515248 | 0.0582 | 0.0398 | 1.0279 | 1.0279 | 1 | 1 |
| WCM80 | 16 | 66908 | 50106594 | 0.034 | −0.0058 | 0.996 | 0.996 | 1 | 1 |
| WCM80 | 16 | 50109478 | 66620997 | −0.4279 | −1.0582 | 0.904 | 0.0564 | 1 | 0 |
| WCM80 | 16 | 66621996 | 81744842 | 0.0192 | −0.0337 | 0.9769 | 0.9769 | 1 | 1 |
| WCM80 | 16 | 81745317 | 90128849 | −0.3805 | −0.9271 | 0.8 | 0.2519 | 1 | 0 |
| WCM80 | 16 | 90130039 | 90168857 | 0.0696 | 0.0609 | NA | NA | NA | NA |
| WCM80 | 17 | 131595 | 81187930 | 0.0369 | −2.00E−04 | 0.9998 | 0.9998 | 1 | 1 |
| WCM80 | 18 | 47680 | 78005267 | 0.0122 | −0.0471 | 0.9679 | 0.9679 | 1 | 1 |
| WCM80 | 19 | 105021 | 59093696 | 0.0639 | 0.0504 | 1.0355 | 1.0355 | 1 | 1 |
| WCM80 | 2 | 41658 | 211542575 | 0.0225 | −0.0274 | 0.9812 | 0.9812 | 1 | 1 |
| WCM80 | 2 | 212240652 | 217366164 | −0.3564 | −0.8633 | 0.9864 | 0.113 | 1 | 0 |
| WCM80 | 2 | 217443557 | 243037103 | 0.0355 | −0.0028 | 0.998 | 0.998 | 1 | 1 |
| WCM80 | 20 | 68299 | 62934572 | 0.0452 | 0.0154 | 1.0107 | 1.0107 | 1 | 1 |
| WCM80 | 21 | 9590305 | 48084189 | 0.006 | −0.0591 | 0.9654 | 0.9543 | 1 | 1 |
| WCM80 | 22 | 16159016 | 17528394 | 0.5175 | 0.8037 | NA | NA | NA | NA |
| WCM80 | 22 | 17577957 | 20290883 | −0.4152 | −1.0224 | 0.9777 | 0.0069 | 1 | 0 |
| WCM80 | 22 | 20291208 | 51223730 | 0.0592 | 0.0415 | 1.0292 | 1.0292 | 1 | 1 |
| WCM80 | 3 | 239390 | 197896724 | 0.0306 | −0.0122 | 0.9916 | 0.9916 | 1 | 1 |
| WCM80 | 4 | 53377 | 174253209 | 0.0132 | −0.0453 | 0.9691 | 0.9691 | 1 | 1 |
| WCM80 | 4 | 174254091 | 190904482 | −0.5256 | −1.3559 | 0.8378 | −0.0564 | 1 | 0 |
| WCM80 | 5 | 90647 | 180687804 | 0.0264 | −0.02 | 0.9862 | 0.9862 | 1 | 1 |
| WCM80 | 6 | 203397 | 170892848 | 0.0153 | −0.0412 | 0.9719 | 0.9719 | 1 | 1 |
| WCM80 | 7 | 193265 | 158937161 | 0.0203 | −0.0316 | 0.9783 | 0.9783 | 1 | 1 |
| WCM80 | 8 | 163457 | 2092641 | −0.4777 | −1.2047 | 0.9529 | −0.0852 | 1 | 0 |
| WCM80 | 8 | 2092921 | 3919229 | 0.4394 | 0.6843 | 1.9187 | 1.2952 | 2 | 1 |
| WCM80 | 8 | 4255373 | 42911666 | −0.4847 | −1.2261 | 0.848 | 0.0069 | 1 | 0 |
| WCM80 | 8 | 42914223 | 146279434 | 0.379 | 0.5895 | 1.8324 | 1.1771 | 2 | 1 |
| WCM80 | 9 | 14880 | 3012996 | 0.0024 | −0.0661 | 1.8947 | 0.0158 | 2 | 0 |
| WCM80 | 9 | 3014100 | 4703077 | −0.9891 | −4.6541 | 0.1166 | −0.0372 | 0 | 0 |
| WCM80 | 9 | 4706214 | 8507472 | 0.0289 | −0.0152 | 2.0102 | −0.0312 | 2 | 0 |
| WCM80 | 9 | 8517899 | 141107533 | 0.03 | −0.0132 | 0.9909 | 0.9909 | 1 | 1 |
| WCM80 | X | 2700110 | 154493609 | −0.0598 | −0.1884 | NA | NA | NA | NA |
| WCM80_Z1_1 | 1 | 69590 | 9932071 | 0.0291 | 0.0218 | 1.0489 | 0.9816 | 1 | 1 |
| WCM80_Z1_1 | 1 | 9990413 | 11204736 | −0.5711 | −0.9523 | 1.0221 | 0.0115 | 1 | 0 |
| WCM80_Z1_1 | 1 | 11205053 | 226128842 | 0.0138 | −3.00E−04 | 1.0114 | 0.9882 | 1 | 1 |
| WCM80_Z1_1 | 1 | 226173146 | 227968259 | −0.6038 | −1.0141 | 0.991 | −7.00E−04 | 1 | 0 |
| WCM80_Z1_1 | 1 | 228003508 | 249212366 | 0.0031 | −0.0158 | 1.002 | 0.9761 | 1 | 1 |
| WCM80_Z1_1 | 10 | 225981 | 135440105 | 0.0163 | 0.0034 | 1.0125 | 0.9922 | 1 | 1 |
| WCM80_Z1_1 | 11 | 193124 | 33902990 | 0.0118 | −0.0032 | 1.0152 | 0.9804 | 1 | 1 |
| WCM80_Z1_1 | 11 | 34073997 | 48328644 | −0.5589 | −0.9296 | 1.0317 | 0.0184 | 1 | 0 |
| WCM80_Z1_1 | 11 | 48346822 | 124135662 | 0.0091 | −0.0071 | 1.0081 | 0.9821 | 1 | 1 |
| WCM80_Z1_1 | 11 | 124179835 | 125366466 | −0.539 | −0.8929 | 1.0937 | −0.0166 | 1 | 0 |
| WCM80_Z1_1 | 11 | 125366680 | 134856536 | 0.0117 | −0.0034 | 1.0309 | 0.9644 | 1 | 1 |
| WCM80_Z1_1 | 12 | 148642 | 104151962 | 0.0093 | −0.0067 | 1.0517 | 0.939 | 1 | 1 |
| WCM80_Z1_1 | 12 | 104153004 | 104492158 | −0.5435 | −0.9012 | NA | NA | NA | NA |
| WCM80_Z1_1 | 12 | 104495751 | 105467741 | −0.0386 | −0.0768 | NA | NA | NA | NA |
| WCM80_Z1_1 | 12 | 105501594 | 108589662 | −0.5318 | −0.8797 | 1.0111 | 0.0759 | 1 | 0 |
| WCM80_Z1_1 | 12 | 108589886 | 110956484 | 0.0189 | 0.007 | 1.0296 | 0.9802 | 1 | 1 |
| WCM80_Z1_1 | 12 | 110957663 | 112035573 | −0.571 | −0.952 | NA | NA | NA | NA |
| WCM80_Z1_1 | 12 | 112036794 | 113624746 | 0.0179 | 0.0056 | 1.0984 | 0.9095 | 1 | 1 |
| WCM80_Z1_1 | 12 | 113629230 | 114841579 | −0.5818 | −0.9723 | NA | NA | NA | NA |
| WCM80_Z1_1 | 12 | 115109666 | 124209332 | 0.0094 | −0.0067 | 1.0532 | 0.9376 | 1 | 1 |
| WCM80_Z1_1 | 12 | 124210786 | 125619355 | −0.5642 | −0.9394 | 1.068 | −0.0251 | 1 | 0 |
| WCM80_Z1_1 | 12 | 125621409 | 133810703 | 0.0127 | −0.0018 | 1.0161 | 0.9814 | 1 | 1 |
| WCM80_Z1_1 | 13 | 19600371 | 33091980 | 0.0183 | 0.0062 | 1.0319 | 0.9767 | 1 | 1 |
| WCM80_Z1_1 | 13 | 33095529 | 76055820 | −0.5996 | −1.006 | 0.9791 | 0.0167 | 1 | 0 |
| WCM80_Z1_1 | 13 | 76101983 | 115089411 | −0.0015 | −0.0224 | 1.0046 | 0.9645 | 1 | 1 |
| WCM80_Z1_1 | 14 | 19377733 | 107283210 | 0.0109 | −0.0045 | 1.0041 | 0.9897 | 1 | 1 |
| WCM80_Z1_1 | 15 | 20170057 | 27018858 | 0.4307 | 0.5708 | 2.0657 | 0.905 | 2 | 1 |
| WCM80_Z1_1 | 15 | 27114425 | 45059946 | −0.5745 | −0.9586 | 1.0437 | −0.0146 | 1 | 0 |
| WCM80_Z1_1 | 15 | 45249116 | 45492969 | −1.6963 | NA | NA | NA | NA | NA |
| WCM80_Z1_1 | 15 | 45544449 | 45782955 | −0.6003 | −1.0074 | NA | NA | NA | NA |
| WCM80_Z1_1 | 15 | 45803456 | 102517641 | 0.0058 | −0.012 | 1.0062 | 0.9773 | 1 | 1 |
| WCM80_Z1_1 | 16 | 67604 | 49856592 | 0.0274 | 0.0193 | 1.0252 | 1.0018 | 1 | 1 |
| WCM80_Z1_1 | 16 | 50059580 | 66647013 | −0.5861 | −0.9805 | 1.0327 | −0.019 | 1 | 0 |
| WCM80_Z1_1 | 16 | 66651247 | 81743404 | 0.0183 | 0.0063 | 1.0276 | 0.9811 | 1 | 1 |
| WCM80_Z1_1 | 16 | 81816733 | 90162178 | −0.5679 | −0.9464 | 0.8171 | 0.2207 | 1 | 0 |
| WCM80_Z1_1 | 17 | 131595 | 81083567 | 0.0146 | 9.00E−04 | 1.0383 | 0.963 | 1 | 1 |
| WCM80_Z1_1 | 18 | 48206 | 78005215 | 0.0557 | 0.0601 | 1.0724 | 1.0127 | 1 | 1 |
| WCM80_Z1_1 | 19 | 281422 | 59083985 | 0.0146 | 8.00E−04 | 1.0513 | 0.9498 | 1 | 1 |
| WCM80_Z1_1 | 2 | 41609 | 215878343 | 0.0055 | −0.0123 | 1.003 | 0.98 | 1 | 1 |
| WCM80_Z1_1 | 2 | 215880324 | 217366061 | −0.5923 | −0.9921 | NA | NA | NA | NA |
| WCM80_Z1_1 | 2 | 217498633 | 242946607 | 0.0207 | 0.0097 | 1.0257 | 0.9878 | 1 | 1 |
| WCM80_Z1_1 | 20 | 68406 | 62919118 | 0.0222 | 0.0119 | 1.02 | 0.9966 | 1 | 1 |
| WCM80_Z1_1 | 21 | 9907291 | 48084204 | −0.0088 | −0.0332 | 0.9958 | 0.9587 | 1 | 1 |
| WCM80_Z1_1 | 22 | 16287449 | 17488945 | 0.3694 | 0.4904 | NA | NA | NA | NA |
| WCM80_Z1_1 | 22 | 17566091 | 20255183 | −0.5528 | −0.9182 | 1.0174 | 0.0409 | 1 | 0 |
| WCM80_Z1_1 | 22 | 20302698 | 51215137 | 0.0279 | 0.0201 | 1.0711 | 0.957 | 1 | 1 |
| WCM80_Z1_1 | 3 | 239350 | 197894712 | 0.0068 | −0.0104 | 1.0029 | 0.9827 | 1 | 1 |
| WCM80_Z1_1 | 4 | 59390 | 174312482 | −4.00E−04 | −0.0209 | 0.9986 | 0.9727 | 1 | 1 |
| WCM80_Z1_1 | 4 | 174448142 | 190873327 | −0.6063 | −1.0188 | 0.9658 | 0.0212 | 1 | 0 |
| WCM80_Z1_1 | 5 | 140532 | 180687808 | 0.0055 | −0.0123 | 1.0059 | 0.9771 | 1 | 1 |
| WCM80_Z1_1 | 6 | 203695 | 170893450 | 0.0014 | −0.0183 | 0.9989 | 0.9759 | 1 | 1 |
| WCM80_Z1_1 | 7 | 193451 | 158937375 | 0.0121 | −0.0028 | 1.0096 | 0.9865 | 1 | 1 |
| WCM80_Z1_1 | 8 | 190907 | 2092866 | −0.6092 | −1.0243 | NA | NA | NA | NA |
| WCM80_Z1_1 | 8 | 2796235 | 3889551 | 0.3922 | 0.5205 | NA | NA | NA | NA |
| WCM80_Z1_1 | 8 | 4277559 | 42873514 | −0.5705 | −0.9512 | 1.0333 | 0.0011 | 1 | 0 |
| WCM80_Z1_1 | 8 | 42911680 | 87242373 | 0.4193 | 0.5559 | 1.9806 | 0.9595 | 2 | 1 |
| WCM80_Z1_1 | 8 | 87386317 | 90802584 | −7.00E−04 | −0.0214 | NA | NA | NA | NA |
| WCM80_Z1_1 | 8 | 90921923 | 146279480 | 0.4222 | 0.5598 | 1.9683 | 0.9797 | 2 | 1 |
| WCM80_Z1_1 | 9 | 17110 | 2838500 | −0.0278 | −0.061 | 1.9511 | −0.034 | 2 | 0 |
| WCM80_Z1_1 | 9 | 3225187 | 4544670 | −1.675 | −12.1435 | NA | NA | NA | NA |
| WCM80_Z1_1 | 9 | 4561454 | 4646492 | 0.0543 | 0.0581 | NA | NA | NA | NA |
| WCM80_Z1_1 | 9 | 4656051 | 4701874 | −1.3652 | −3.2124 | NA | NA | NA | NA |
| WCM80_Z1_1 | 9 | 4706022 | 8525041 | −0.0147 | −0.0418 | NA | NA | NA | NA |
| WCM80_Z1_1 | 9 | 8526630 | 141111414 | 0.0139 | −1.00E−04 | 1.0057 | 0.9941 | 1 | 1 |
| WCM80_Z1_1 | X | 2700119 | 154509348 | −0.0264 | −0.0589 | NA | NA | NA | NA |
| WCM89 | 1 | 14930 | 249214028 | 0.0328 | NA | NA | NA | NA | NA |
| WCM89 | 10 | 226021 | 135477913 | 0.0274 | NA | NA | NA | NA | NA |
| WCM89 | 11 | 193096 | 134605916 | 0.0252 | NA | NA | NA | NA | NA |
| WCM89 | 12 | 148480 | 133810703 | 0.0102 | NA | NA | NA | NA | NA |
| WCM89 | 13 | 19041994 | 115089411 | 0.0198 | NA | NA | NA | NA | NA |
| WCM89 | 14 | 19110178 | 107283266 | 0.0235 | NA | NA | NA | NA | NA |
| WCM89 | 15 | 20083896 | 102506257 | 0.0403 | NA | NA | NA | NA | NA |
| WCM89 | 16 | 66905 | 90168857 | 0.0216 | NA | NA | NA | NA | NA |
| WCM89 | 17 | 131595 | 81187845 | 0.0256 | NA | NA | NA | NA | NA |
| WCM89 | 18 | 47738 | 78005205 | 0.0162 | NA | NA | NA | NA | NA |
| WCM89 | 19 | 105080 | 59093696 | 0.0042 | NA | NA | NA | NA | NA |
| WCM89 | 2 | 41602 | 243037022 | 0.0216 | NA | NA | NA | NA | NA |
| WCM89 | 20 | 68346 | 270236 | 0.0386 | NA | NA | NA | NA | NA |
| WCM89 | 20 | 271168 | 278806 | −1.4078 | NA | NA | NA | NA | NA |
| WCM89 | 20 | 278983 | 62934617 | 0.0425 | NA | NA | NA | NA | NA |
| WCM89 | 21 | 9590314 | 48084204 | 0.0192 | NA | NA | NA | NA | NA |
| WCM89 | 22 | 16159060 | 51223730 | 0.0617 | NA | NA | NA | NA | NA |
| WCM89 | 3 | 239531 | 197896705 | 0.0309 | NA | NA | NA | NA | NA |
| WCM89 | 4 | 53345 | 190878479 | 0.0148 | NA | NA | NA | NA | NA |
| WCM89 | 5 | 90684 | 180687847 | 0.0158 | NA | NA | NA | NA | NA |
| WCM89 | 6 | 203369 | 170892854 | 0.0112 | NA | NA | NA | NA | NA |
| WCM89 | 7 | 193254 | 158937161 | 0.0078 | NA | NA | NA | NA | NA |
| WCM89 | 8 | 163438 | 146279489 | 0.0106 | NA | NA | NA | NA | NA |
| WCM89 | 9 | 14893 | 141107582 | 0.0332 | NA | NA | NA | NA | NA |
| WCM89 | X | 2700166 | 154493598 | −0.1228 | NA | NA | NA | NA | NA |
| WCM89_X1_1 | 1 | 69590 | 38005914 | −0.0279 | −0.2393 | 0.9142 | 0.7802 | 1 | 1 |
| WCM89_X1_1 | 1 | 38006256 | 38463196 | −0.699 | −1.5195 | 0.6559 | 0.0417 | 1 | 0 |
| WCM89_X1_1 | 1 | 38463504 | 79093646 | 0.0094 | −0.1808 | 0.9262 | 0.8382 | 1 | 1 |
| WCM89_X1_1 | 1 | 79093994 | 85666196 | 0.2327 | 0.1536 | NA | NA | NA | NA |
| WCM89_X1_1 | 1 | 85724328 | 120438136 | 0.1159 | −0.018 | 1.1696 | 0.8055 | 1 | 1 |
| WCM89_X1_1 | 1 | 120438468 | 180471187 | 0.277 | 0.2171 | 1.1922 | 1.1326 | 1 | 1 |
| WCM89_X1_1 | 1 | 180601391 | 193119454 | −0.1236 | −0.3937 | 1.022 | 0.5004 | 1 | 1 |
| WCM89_X1_1 | 1 | 193121514 | 220253100 | 0.0916 | −0.0546 | 0.9643 | 0.9614 | 1 | 1 |
| WCM89_X1_1 | 1 | 220267657 | 249212434 | −0.1673 | −0.4665 | 0.9925 | 0.4549 | 1 | 0 |
| WCM89_X1_1 | 10 | 225958 | 85902454 | 0.1293 | 0.002 | 1.0014 | 1.0014 | 1 | 1 |
| WCM89_X1_1 | 10 | 85903786 | 111628425 | −0.5217 | −1.124 | 0.9364 | −0.0188 | 1 | 0 |
| WCM89_X1_1 | 10 | 111629723 | 135440081 | 0.0465 | −0.1233 | 0.9181 | 0.9181 | 1 | 1 |
| WCM89_X1_1 | 11 | 193112 | 55563900 | 0.0856 | −0.0638 | 0.9712 | 0.9423 | 1 | 1 |
| WCM89_X1_1 | 11 | 55579118 | 134215007 | −0.1744 | −0.4784 | 0.9526 | 0.483 | 1 | 0 |
| WCM89_X1_1 | 11 | 134217298 | 134856574 | 0.0732 | −0.0826 | NA | NA | NA | NA |
| WCM89_X1_1 | 12 | 148811 | 16507249 | −0.3549 | −0.7972 | 1.1874 | −0.0365 | 1 | 0 |
| WCM89_X1_1 | 12 | 16516928 | 78582090 | −0.0609 | −0.2919 | 1.1559 | 0.4777 | 1 | 0 |
| WCM89_X1_1 | 12 | 78582402 | 93881418 | 0.2723 | 0.2103 | 1.2625 | 1.0514 | 1 | 1 |
| WCM89_X1_1 | 12 | 93897959 | 123694661 | −0.0864 | −0.333 | 1.1052 | 0.4826 | 1 | 0 |
| WCM89_X1_1 | 12 | 123699304 | 133810703 | −0.3162 | −0.7262 | 1.2035 | 0.0055 | 1 | 0 |
| WCM89_X1_1 | 13 | 19600515 | 115089411 | −0.3999 | −0.8819 | 0.6193 | 0.4661 | 1 | 0 |
| WCM89_X1_1 | 14 | 19377692 | 107283184 | 0.1228 | −0.0077 | 0.9991 | 0.9902 | 1 | 1 |
| WCM89_X1_1 | 15 | 20170123 | 102506284 | 0.1239 | −0.0062 | 1.0062 | 0.9853 | 1 | 1 |
| WCM89_X1_1 | 16 | 67604 | 33020784 | 0.3636 | 0.3389 | 1.5055 | 1.024 | 2 | 1 |
| WCM89_X1_1 | 16 | 33500782 | 90162133 | −0.1072 | −0.3668 | 1.0265 | 0.5244 | 1 | 1 |
| WCM89_X1_1 | 17 | 131595 | 20483882 | −0.4152 | −0.9114 | 1.0387 | 0.0247 | 1 | 0 |
| WCM89_X1_1 | 17 | 20798992 | 81083455 | 0.1509 | 0.034 | 1.0314 | 1.0162 | 1 | 1 |
| WCM89_X1_1 | 18 | 49007 | 7034508 | −0.0951 | −0.3472 | 0.9817 | 0.5905 | 1 | 1 |
| WCM89_X1_1 | 18 | 7036028 | 13387727 | 0.2845 | 0.2278 | 1.878 | 0.464 | 2 | 0 |
| WCM89_X1_1 | 18 | 13438274 | 19751474 | −0.5299 | −1.1411 | NA | NA | NA | NA |
| WCM89_X1_1 | 18 | 19752089 | 28648063 | 0.2611 | 0.1945 | 1.8678 | 0.4208 | 2 | 0 |
| WCM89_X1_1 | 18 | 28648310 | 78005210 | −0.1696 | −0.4704 | 0.9076 | 0.536 | 1 | 1 |
| WCM89_X1_1 | 19 | 281435 | 59083951 | 0.1124 | −0.0233 | 0.9869 | 0.981 | 1 | 1 |
| WCM89_X1_1 | 2 | 41613 | 286163 | −0.0245 | −0.234 | NA | NA | NA | NA |
| WCM89_X1_1 | 2 | 287719 | 8867065 | −0.1473 | −0.4329 | 1.0651 | 0.4164 | 1 | 0 |
| WCM89_X1_1 | 2 | 8870862 | 10595578 | 0.5375 | 0.5752 | NA | NA | NA | NA |
| WCM89_X1_1 | 2 | 10712300 | 89161435 | −0.145 | −0.4292 | 1.0107 | 0.4746 | 1 | 0 |
| WCM89_X1_1 | 2 | 89185094 | 242946616 | 0.1126 | −0.023 | 0.996 | 0.9723 | 1 | 1 |
| WCM89_X1_1 | 20 | 68397 | 62904835 | 0.0874 | −0.0611 | 0.9832 | 0.9339 | 1 | 1 |
| WCM89_X1_1 | 21 | 9907295 | 39795444 | 0.1742 | 0.0684 | 1.0901 | 1.0069 | 1 | 1 |
| WCM89_X1_1 | 21 | 39817489 | 42870081 | −0.2932 | −0.6847 | 1.1471 | 0.0971 | 1 | 0 |
| WCM89_X1_1 | 21 | 43133061 | 48084210 | 0.2466 | 0.1736 | 1.1489 | 1.1068 | 1 | 1 |
| WCM89_X1_1 | 22 | 17063476 | 51215120 | 0.124 | −0.006 | 1.0078 | 0.984 | 1 | 1 |
| WCM89_X1_1 | 3 | 239347 | 60522697 | −0.1032 | −0.3603 | 1.0282 | 0.5298 | 1 | 1 |
| WCM89_X1_1 | 3 | 61548006 | 67054317 | 0.5708 | 0.6194 | NA | NA | NA | NA |
| WCM89_X1_1 | 3 | 67054523 | 75986717 | −0.444 | −0.9673 | 1.1159 | −0.0929 | 1 | 0 |
| WCM89_X1_1 | 3 | 77089988 | 195937552 | 0.1507 | 0.0337 | 1.0358 | 1.0115 | 1 | 1 |
| WCM89_X1_1 | 3 | 195938177 | 197896720 | −0.0427 | −0.2628 | 0.9382 | 0.7287 | 1 | 1 |
| WCM89_X1_1 | 4 | 59397 | 48588742 | −0.3048 | −0.7056 | 0.7779 | 0.4484 | 1 | 0 |
| WCM89_X1_1 | 4 | 48591778 | 190426502 | −0.0746 | −0.314 | 0.9056 | 0.7033 | 1 | 1 |
| WCM89_X1_1 | 5 | 140559 | 180687776 | 0.0384 | −0.1359 | 0.9213 | 0.8989 | 1 | 1 |
| WCM89_X1_1 | 6 | 203552 | 7727404 | −0.6648 | −1.4384 | 0.5663 | 0.1717 | 1 | 0 |
| WCM89_X1_1 | 6 | 7727750 | 170893438 | −0.1689 | −0.4691 | 0.9702 | 0.4745 | 1 | 0 |
| WCM89_X1_1 | 7 | 193222 | 39649510 | 0.0603 | −0.1023 | 0.9501 | 0.913 | 1 | 1 |
| WCM89_X1_1 | 7 | 39726338 | 158937416 | −0.1982 | −0.5187 | 0.9527 | 0.4432 | 1 | 0 |
| WCM89_X1_1 | 8 | 190897 | 17924739 | −0.2095 | −0.5381 | 0.9674 | 0.4099 | 1 | 0 |
| WCM89_X1_1 | 8 | 17927304 | 28420497 | −0.9267 | −2.1459 | 0.476 | −0.0241 | 0 | 0 |
| WCM89_X1_1 | 8 | 28573703 | 48874714 | −0.2359 | −0.5837 | 0.9155 | 0.4191 | 1 | 0 |
| WCM89_X1_1 | 8 | 48874963 | 66619386 | 0.367 | 0.3436 | 1.3535 | 1.1844 | 1 | 1 |
| WCM89_X1_1 | 8 | 66620187 | 86027716 | 0.8716 | 1.0047 | 3.1161 | 0.8969 | 3 | 1 |
| WCM89_X1_1 | 8 | 86035738 | 92407324 | 0.4433 | 0.4485 | 1.7584 | 0.9709 | 2 | 1 |
| WCM89_X1_1 | 8 | 92972719 | 114627743 | −0.1154 | −0.3802 | 1.0709 | 0.4657 | 1 | 0 |
| WCM89_X1_1 | 8 | 116426335 | 143602208 | 0.2541 | 0.1844 | 1.4328 | 0.8399 | 1 | 1 |
| WCM89_X1_1 | 8 | 143603425 | 146279423 | −0.345 | −0.7789 | 0.814 | 0.3516 | 1 | 0 |
| WCM89_X1_1 | 9 | 17066 | 19378494 | −0.4428 | −0.9648 | 1.0288 | −0.0042 | 1 | 0 |
| WCM89_X1_1 | 9 | 19378751 | 71152348 | 0.1131 | −0.0223 | 1.0039 | 0.9655 | 1 | 1 |
| WCM89_X1_1 | 9 | 71155503 | 86414206 | −0.5354 | −1.1525 | 0.8588 | 0.0408 | 1 | 0 |
| WCM89_X1_1 | 9 | 86421335 | 141111414 | 0.0343 | −0.1422 | 0.9117 | 0.9005 | 1 | 1 |
| WCM89_X1_1 | X | 2707716 | 19027799 | 0.0057 | −0.1865 | NA | NA | NA | NA |
| WCM89_X1_1 | X | 19032163 | 48791818 | −0.4677 | −1.0143 | NA | NA | NA | NA |
| WCM89_X1_1 | X | 48792099 | 63410086 | 0.1762 | 0.0713 | NA | NA | NA | NA |
| WCM89_X1_1 | X | 63410555 | 69282985 | 1.7947 | 2.0862 | NA | NA | NA | NA |
| WCM89_X1_1 | X | 69353804 | 154509348 | 0.1597 | 0.0471 | NA | NA | NA | NA |
| WCM89_Z1_1 | 1 | 69594 | 917492 | 0.0777 | 0.2591 | NA | NA | NA | NA |
| WCM89_Z1_1 | 1 | 935081 | 37980309 | −0.0466 | −0.0455 | 1.0804 | 0.8575 | 1 | 1 |
| WCM89_Z1_1 | 1 | 38003554 | 38463504 | −0.405 | −1.2804 | 0.9491 | −0.1257 | 1 | 0 |
| WCM89_Z1_1 | 1 | 38463706 | 85136473 | −0.0251 | 0.01 | 1.0429 | 0.971 | 1 | 1 |
| WCM89_Z1_1 | 1 | 85136884 | 249212366 | 0.1218 | 0.359 | 1.3167 | 1.2484 | 1 | 1 |
| WCM89_Z1_1 | 10 | 225979 | 85901313 | 0.0503 | 0.1952 | 1.1558 | 1.1341 | 1 | 1 |
| WCM89_Z1_1 | 10 | 85902478 | 111629778 | −0.3054 | −0.8546 | 1.1056 | 4.00E−04 | 1 | 0 |
| WCM89_Z1_1 | 10 | 111630651 | 135440105 | 0.0194 | 0.121 | 1.1114 | 1.0636 | 1 | 1 |
| WCM89_Z1_1 | 11 | 193112 | 134856559 | 0.047 | 0.1874 | 1.1549 | 1.1225 | 1 | 1 |
| WCM89_Z1_1 | 12 | 148644 | 16758746 | −0.2854 | −0.7797 | 1.2265 | −0.0615 | 1 | 0 |
| WCM89_Z1_1 | 12 | 17142198 | 78593284 | −0.0917 | −0.1667 | 1.21 | 0.5717 | 1 | 1 |
| WCM89_Z1_1 | 12 | 78598894 | 94287893 | 0.0949 | 0.2986 | 1.3501 | 1.1098 | 1 | 1 |
| WCM89_Z1_1 | 12 | 94543029 | 123694675 | −0.1188 | −0.2426 | 1.1657 | 0.5248 | 1 | 1 |
| WCM89_Z1_1 | 12 | 123699304 | 133810703 | −0.2617 | −0.694 | 1.2575 | −0.0213 | 1 | 0 |
| WCM89_Z1_1 | 13 | 19600475 | 38225461 | −0.2986 | −0.8289 | 0.5949 | 0.5311 | 1 | 1 |
| WCM89_Z1_1 | 13 | 38229294 | 115089411 | −0.1559 | −0.3508 | 0.8405 | 0.7278 | 1 | 1 |
| WCM89_Z1_1 | 14 | 19377689 | 107283027 | 0.0354 | 0.1596 | 1.1304 | 1.1036 | 1 | 1 |
| WCM89_Z1_1 | 15 | 20170129 | 102506273 | 0.0441 | 0.1805 | 1.1495 | 1.1171 | 1 | 1 |
| WCM89_Z1_1 | 16 | 67604 | 33020700 | 0.0641 | 0.2277 | 1.201 | 1.1409 | 1 | 1 |
| WCM89_Z1_1 | 16 | 33500782 | 90233627 | −0.1178 | −0.2398 | 1.1507 | 0.5431 | 1 | 1 |
| WCM89_Z1_1 | 17 | 131595 | 21191346 | −0.3062 | −0.8577 | 1.1908 | −0.0872 | 1 | 0 |
| WCM89_Z1_1 | 17 | 21194820 | 81083507 | 0.0523 | 0.1999 | 1.1486 | 1.1486 | 1 | 1 |
| WCM89_Z1_1 | 18 | 49036 | 78005219 | −0.0226 | 0.0163 | 1.4234 | 0.5993 | 1 | 1 |
| WCM89_Z1_1 | 19 | 281483 | 59083951 | 0.0336 | 0.1555 | 1.1299 | 1.0977 | 1 | 1 |
| WCM89_Z1_1 | 2 | 41623 | 8822518 | −0.1996 | −0.4859 | 0.7961 | 0.632 | 1 | 1 |
| WCM89_Z1_1 | 2 | 8822985 | 10594161 | 0.213 | 0.5541 | NA | NA | NA | NA |
| WCM89_Z1_1 | 2 | 10595578 | 60688446 | −0.1788 | −0.4205 | 0.8003 | 0.694 | 1 | 1 |
| WCM89_Z1_1 | 2 | 60688928 | 62934335 | 0.1741 | 0.4725 | 2.4826 | 0.2925 | 2 | 0 |
| WCM89_Z1_1 | 2 | 62998533 | 96942672 | −0.1733 | −0.4037 | 0.7954 | 0.7165 | 1 | 1 |
| WCM89_Z1_1 | 2 | 96942929 | 96992374 | 0.0781 | 0.26 | NA | NA | NA | NA |
| WCM89_Z1_1 | 2 | 96992814 | 152127328 | −0.0145 | 0.0368 | 1.0673 | 0.9844 | 1 | 1 |
| WCM89_Z1_1 | 2 | 152128190 | 242946616 | 0.1089 | 0.3301 | 1.5279 | 0.9863 | 2 | 1 |
| WCM89_Z1_1 | 20 | 68373 | 62904845 | −0.0364 | −0.0192 | 0.997 | 0.9766 | 1 | 1 |
| WCM89_Z1_1 | 21 | 9907301 | 39822803 | 0.0547 | 0.2057 | 1.2157 | 1.0907 | 1 | 1 |
| WCM89_Z1_1 | 21 | 39947586 | 43133829 | −0.2935 | −0.8099 | 0.9952 | 0.1457 | 1 | 0 |
| WCM89_Z1_1 | 21 | 43134212 | 48084204 | 0.0491 | 0.1923 | 1.24 | 1.0451 | 1 | 1 |
| WCM89_Z1_1 | 22 | 16287509 | 51215120 | 0.0429 | 0.1775 | 1.1417 | 1.1202 | 1 | 1 |
| WCM89_Z1_1 | 3 | 239459 | 67054185 | 0.0543 | 0.2047 | 1.1524 | 1.1524 | 1 | 1 |
| WCM89_Z1_1 | 3 | 67054451 | 74548852 | −0.323 | −0.9232 | 1.0544 | 3.00E−04 | 1 | 0 |
| WCM89_Z1_1 | 3 | 75263631 | 197896714 | 0.0488 | 0.1917 | 1.1448 | 1.1394 | 1 | 1 |
| WCM89_Z1_1 | 4 | 59394 | 102712108 | 0.0063 | 0.0889 | 1.0688 | 1.0583 | 1 | 1 |
| WCM89_Z1_1 | 4 | 102751034 | 129776829 | −0.1692 | −0.391 | 0.8464 | 0.6789 | 1 | 1 |
| WCM89_Z1_1 | 4 | 129778510 | 190426502 | 0.0018 | 0.0776 | 1.0867 | 1.0238 | 1 | 1 |
| WCM89_Z1_1 | 5 | 140477 | 180687804 | 0.0489 | 0.1919 | 1.1477 | 1.1369 | 1 | 1 |
| WCM89_Z1_1 | 6 | 203479 | 7845478 | −0.3923 | −1.2203 | 0.485 | 0.3734 | 0 | 0 |
| WCM89_Z1_1 | 6 | 7861696 | 170893409 | −0.1139 | −0.2286 | 1.204 | 0.5029 | 1 | 1 |
| WCM89_Z1_1 | 7 | 193254 | 39730022 | 0.0413 | 0.1738 | 1.1495 | 1.1065 | 1 | 1 |
| WCM89_Z1_1 | 7 | 39736391 | 158937400 | −0.12 | −0.246 | 1.1923 | 0.4942 | 1 | 0 |
| WCM89_Z1_1 | 8 | 190900 | 17924729 | −0.078 | −0.129 | 1.1474 | 0.6815 | 1 | 1 |
| WCM89_Z1_1 | 8 | 17927312 | 28420410 | −0.5042 | −1.834 | 0.5424 | 0.0186 | 1 | 0 |
| WCM89_Z1_1 | 8 | 28573750 | 52321623 | −0.1286 | −0.2706 | 0.9487 | 0.7093 | 1 | 1 |
| WCM89_Z1_1 | 8 | 52321776 | 70539449 | 0.1408 | 0.4008 | 1.6347 | 1.0059 | 2 | 1 |
| WCM89_Z1_1 | 8 | 70540006 | 85785606 | 0.5618 | 1.2002 | 3.2052 | 1.3902 | 3 | 1 |
| WCM89_Z1_1 | 8 | 85799889 | 88296935 | 0.2562 | 0.6419 | 1.7073 | 1.4136 | 2 | 1 |
| WCM89_Z1_1 | 8 | 88298843 | 107630406 | −0.0908 | −0.164 | 1.1996 | 0.5855 | 1 | 1 |
| WCM89_Z1_1 | 8 | 107670297 | 114448958 | 0.1107 | 0.3342 | 1.4273 | 1.094 | 1 | 1 |
| WCM89_Z1_1 | 8 | 114612663 | 143694768 | 0.3171 | 0.7615 | 1.7474 | 1.6431 | 2 | 2 |
| WCM89_Z1_1 | 8 | 143695144 | 145171114 | 0.1001 | 0.3105 | 1.9832 | 0.497 | 2 | 0 |
| WCM89_Z1_1 | 8 | 145218743 | 146279489 | −0.1492 | −0.3308 | NA | NA | NA | NA |
| WCM89_Z1_1 | 9 | 17055 | 19352622 | −0.3083 | −0.8658 | 1.1171 | −0.0196 | 1 | 0 |
| WCM89_Z1_1 | 9 | 19357090 | 71152177 | 0.066 | 0.2321 | 1.232 | 1.117 | 1 | 1 |
| WCM89_Z1_1 | 9 | 71155549 | 86322524 | −0.3105 | −0.8744 | 1.1467 | −0.0557 | 1 | 0 |
| WCM89_Z1_1 | 9 | 86327752 | 141111414 | 0.0601 | 0.2182 | 1.1769 | 1.1497 | 1 | 1 |
| WCM89_Z1_1 | X | 2707682 | 19663526 | −0.0076 | 0.0542 | NA | NA | NA | NA |
| WCM89_Z1_1 | X | 19666174 | 48435411 | −0.3597 | −1.0746 | NA | NA | NA | NA |
| WCM89_Z1_1 | X | 48435890 | 62926275 | 0.0744 | 0.2516 | NA | NA | NA | NA |
| WCM89_Z1_1 | X | 63005022 | 65427997 | 0.5097 | 1.1115 | NA | NA | NA | NA |
| WCM89_Z1_1 | X | 65474912 | 69282771 | 1.9692 | 3.1176 | NA | NA | NA | NA |
| WCM89_Z1_1 | X | 69353859 | 154509348 | 0.0749 | 0.2529 | NA | NA | NA | NA |
| WCM89_Z12_1 | 1 | 69603 | 249212402 | −0.0649 | −0.0022 | 1.0278 | 0.9693 | 1 | 1 |
| WCM89_Z12_1 | 10 | 225988 | 135440081 | −0.0724 | −0.0206 | 0.9979 | 0.9737 | 1 | 1 |
| WCM89_Z12_1 | 11 | 193112 | 134856592 | −0.0666 | −0.0064 | 1.0175 | 0.9737 | 1 | 1 |
| WCM89_Z12_1 | 12 | 148811 | 133810703 | −0.0576 | 0.0155 | 1.0353 | 0.9863 | 1 | 1 |
| WCM89_Z12_1 | 13 | 19600515 | 115089411 | −0.0729 | −0.0219 | 1.0165 | 0.9534 | 1 | 1 |
| WCM89_Z12_1 | 14 | 19377701 | 107283210 | −0.0696 | −0.0137 | 1.1314 | 0.8496 | 1 | 1 |
| WCM89_Z12_1 | 15 | 20170057 | 102506273 | −0.068 | −0.0097 | 1.0273 | 0.9592 | 1 | 1 |
| WCM89_Z12_1 | 16 | 67604 | 90233627 | −0.0628 | 0.0029 | 1.0264 | 0.9776 | 1 | 1 |
| WCM89_Z12_1 | 17 | 131595 | 81083507 | −0.0679 | −0.0095 | 1.0129 | 0.974 | 1 | 1 |
| WCM89_Z12_1 | 18 | 49046 | 78005210 | −0.0835 | −0.0481 | 0.9865 | 0.948 | 1 | 1 |
| WCM89_Z12_1 | 19 | 281499 | 59083923 | −0.0409 | 0.0557 | 1.0641 | 1.0145 | 1 | 1 |
| WCM89_Z12_1 | 2 | 41623 | 242946616 | −0.0669 | −0.0071 | 1.0073 | 0.9829 | 1 | 1 |
| WCM89_Z12_1 | 20 | 68375 | 62904861 | −0.0705 | −0.0159 | 1.0133 | 0.9647 | 1 | 1 |
| WCM89_Z12_1 | 21 | 9907316 | 48084214 | −0.0975 | −0.0832 | 0.9869 | 0.9011 | 1 | 1 |
| WCM89_Z12_1 | 22 | 16287479 | 51215134 | −0.0794 | −0.0379 | 1.0006 | 0.9475 | 1 | 1 |
| WCM89_Z12_1 | 3 | 239431 | 197896714 | −0.0752 | −0.0275 | 0.9908 | 0.9714 | 1 | 1 |
| WCM89_Z12_1 | 4 | 59389 | 190426502 | −0.0727 | −0.0214 | 1.0022 | 0.9683 | 1 | 1 |
| WCM89_Z12_1 | 5 | 140515 | 180687768 | −0.0515 | 0.0304 | 1.0434 | 0.9991 | 1 | 1 |
| WCM89_Z12_1 | 6 | 203615 | 170893454 | −0.068 | −0.0098 | 1.0224 | 0.9641 | 1 | 1 |
| WCM89_Z12_1 | 7 | 193205 | 158937400 | −0.0643 | −7.00E−04 | 1.019 | 0.98 | 1 | 1 |
| WCM89_Z12_1 | 8 | 190908 | 4851906 | −0.0263 | 0.0902 | NA | NA | NA | NA |
| WCM89_Z12_1 | 8 | 6266883 | 27650185 | −0.361 | −0.8724 | 1.0962 | −0.0037 | 1 | 0 |
| WCM89_Z12_1 | 8 | 27657086 | 146279511 | −0.0708 | −0.0167 | 1.0152 | 0.9618 | 1 | 1 |
| WCM89_Z12_1 | 9 | 17058 | 141111414 | −0.059 | 0.0122 | 1.0403 | 0.9767 | 1 | 1 |
| WCM89_Z12_1 | X | 2707700 | 154509348 | −0.0289 | 0.0841 | NA | NA | NA | NA |
| WCM89_Z17_1 | 1 | 69610 | 249212348 | −0.0424 | 0.0679 | 1.1654 | 0.931 | 1 | 1 |
| WCM89_Z17_1 | 10 | 225981 | 85909909 | −0.0258 | 0.1079 | 1.1009 | 1.0545 | 1 | 1 |
| WCM89_Z17_1 | 10 | 85910582 | 111639975 | −0.3721 | −0.9222 | 1.0448 | 0.0106 | 1 | 0 |
| WCM89_Z17_1 | 10 | 111640659 | 135440108 | −0.1009 | −0.0786 | 1.0106 | 0.8834 | 1 | 1 |
| WCM89_Z17_1 | 11 | 193112 | 294119 | −0.1405 | −0.1831 | 1.4141 | 0.3475 | 1 | 0 |
| WCM89_Z17_1 | 11 | 294453 | 2683222 | −0.3069 | −0.6864 | 0.9778 | 0.265 | 1 | 0 |
| WCM89_Z17_1 | 11 | 2790078 | 134856562 | −0.0328 | 0.0912 | 1.0729 | 1.0576 | 1 | 1 |
| WCM89_Z17_1 | 12 | 148675 | 16347371 | −0.3313 | −0.7714 | 1.1303 | 0.0415 | 1 | 0 |
| WCM89_Z17_1 | 12 | 16369897 | 123714910 | −0.1291 | −0.1526 | 1.0628 | 0.7365 | 1 | 1 |
| WCM89_Z17_1 | 12 | 123738311 | 133810703 | −0.4298 | −1.1577 | 0.9392 | −0.0427 | 1 | 0 |
| WCM89_Z17_1 | 13 | 19600515 | 115089411 | −0.1298 | −0.1545 | 0.9392 | 0.8577 | 1 | 1 |
| WCM89_Z17_1 | 14 | 19377692 | 107283027 | −0.1525 | −0.2159 | 1.0404 | 0.6816 | 1 | 1 |
| WCM89_Z17_1 | 15 | 20170003 | 102506282 | −0.0888 | −0.0473 | 0.9998 | 0.9356 | 1 | 1 |
| WCM89_Z17_1 | 16 | 67604 | 815266 | −0.1473 | −0.2015 | 0.9858 | 0.7535 | 1 | 1 |
| WCM89_Z17_1 | 16 | 815560 | 49315259 | −0.0794 | −0.0236 | 1.0161 | 0.9515 | 1 | 1 |
| WCM89_Z17_1 | 16 | 49407998 | 90233627 | −0.2291 | −0.4365 | 1.0479 | 0.43 | 1 | 0 |
| WCM89_Z17_1 | 17 | 131595 | 20363675 | −0.3606 | −0.8786 | 1.0324 | 0.0554 | 1 | 0 |
| WCM89_Z17_1 | 17 | 20483308 | 80789885 | −0.0907 | −0.0523 | 0.9965 | 0.9324 | 1 | 1 |
| WCM89_Z17_1 | 17 | 80790093 | 81083455 | −0.3406 | −0.8049 | NA | NA | NA | NA |
| WCM89_Z17_1 | 18 | 49005 | 78005214 | −0.1723 | −0.2707 | 1.0851 | 0.5727 | 1 | 1 |
| WCM89_Z17_1 | 19 | 281491 | 15166802 | −0.1113 | −0.1056 | 0.9683 | 0.8906 | 1 | 1 |
| WCM89_Z17_1 | 19 | 15198024 | 15198606 | −1.9732 | NA | NA | NA | NA | NA |
| WCM89_Z17_1 | 19 | 15198751 | 59083953 | −0.043 | 0.0667 | 1.2154 | 0.8792 | 1 | 1 |
| WCM89_Z17_1 | 2 | 41614 | 127447856 | −0.0695 | 0.0013 | 1.0234 | 0.9784 | 1 | 1 |
| WCM89_Z17_1 | 2 | 127451505 | 225346646 | 0.1149 | 0.4243 | 1.5948 | 1.089 | 2 | 1 |
| WCM89_Z17_1 | 2 | 225360672 | 242946616 | −0.0887 | −0.0473 | 1.0171 | 0.9184 | 1 | 1 |
| WCM89_Z17_1 | 20 | 68406 | 62904881 | −0.0829 | −0.0323 | 0.9952 | 0.9605 | 1 | 1 |
| WCM89_Z17_1 | 21 | 9907297 | 48084226 | −0.16 | −0.2366 | 1.0179 | 0.6797 | 1 | 1 |
| WCM89_Z17_1 | 22 | 16287467 | 51215120 | −0.1505 | −0.2105 | 0.8855 | 0.843 | 1 | 1 |
| WCM89_Z17_1 | 3 | 239350 | 197896711 | −0.0232 | 0.1144 | 1.0877 | 1.0773 | 1 | 1 |
| WCM89_Z17_1 | 4 | 59375 | 190426502 | −0.0763 | −0.0157 | 1.0215 | 0.9568 | 1 | 1 |
| WCM89_Z17_1 | 5 | 140559 | 180687773 | −0.0555 | 0.0359 | 1.0479 | 1.0025 | 1 | 1 |
| WCM89_Z17_1 | 6 | 203515 | 170893427 | −0.1869 | −0.3121 | 0.8331 | 0.7778 | 1 | 1 |
| WCM89_Z17_1 | 7 | 193451 | 158937375 | −0.0611 | 0.0222 | 1.0381 | 0.9929 | 1 | 1 |
| WCM89_Z17_1 | 8 | 190906 | 17924729 | 0.0047 | 0.18 | 1.1355 | 1.1302 | 1 | 1 |
| WCM89_Z17_1 | 8 | 17927313 | 29035028 | −0.3258 | −0.752 | 1.1843 | 0.0033 | 1 | 0 |
| WCM89_Z17_1 | 8 | 29037644 | 146279489 | −0.0209 | 0.1198 | 1.1151 | 1.0582 | 1 | 1 |
| WCM89_Z17_1 | 9 | 17066 | 19360389 | −0.2928 | −0.6386 | 1.0108 | 0.2738 | 1 | 0 |
| WCM89_Z17_1 | 9 | 19361898 | 36170952 | −0.0322 | 0.0928 | 1.1537 | 0.9791 | 1 | 1 |
| WCM89_Z17_1 | 9 | 36171200 | 86243114 | −0.2417 | −0.475 | 1.1323 | 0.3067 | 1 | 0 |
| WCM89_Z17_1 | 9 | 86243799 | 126133804 | −0.018 | 0.1267 | 1.1203 | 1.0633 | 1 | 1 |
| WCM89_Z17_1 | 9 | 126134542 | 141111414 | −0.2806 | −0.5986 | 0.8483 | 0.4724 | 1 | 0 |
| WCM89_Z17_1 | X | 2707700 | 154509348 | 0.0027 | 0.1754 | NA | NA | NA | NA |
| WCM89_ZLN1_1 | 1 | 69592 | 249212366 | −0.0556 | 0.0555 | 1.0579 | 1.0205 | 1 | 1 |
| WCM89_ZLN1_1 | 10 | 225960 | 85904745 | −0.0353 | 0.1073 | 1.0772 | 1.0772 | 1 | 1 |
| WCM89_ZLN1_1 | 10 | 85908571 | 111879244 | −0.3211 | −0.7605 | 0.9636 | 0.217 | 1 | 0 |
| WCM89_ZLN1_1 | 10 | 111881962 | 135440090 | −0.181 | −0.2916 | 0.8586 | 0.7754 | 1 | 1 |
| WCM89_ZLN1_1 | 11 | 193112 | 134856592 | −0.0584 | 0.0485 | 1.0555 | 1.0129 | 1 | 1 |
| WCM89_ZLN1_1 | 12 | 148674 | 14650619 | −0.2948 | −0.6636 | 1.0002 | 0.2625 | 1 | 0 |
| WCM89_ZLN1_1 | 12 | 14650954 | 77440011 | −0.143 | −0.1805 | 1.1288 | 0.636 | 1 | 1 |
| WCM89_ZLN1_1 | 12 | 77440093 | 94658921 | 0.1288 | 0.4891 | 1.5826 | 1.2245 | 2 | 1 |
| WCM89_ZLN1_1 | 12 | 94671859 | 123787393 | −0.1774 | −0.281 | 0.8574 | 0.7887 | 1 | 1 |
| WCM89_ZLN1_1 | 12 | 123789135 | 133810703 | −0.394 | −1.0562 | 0.9499 | 0.0119 | 1 | 0 |
| WCM89_ZLN1_1 | 13 | 19600515 | 115089411 | −0.1122 | −0.0945 | 0.9803 | 0.893 | 1 | 1 |
| WCM89_ZLN1_1 | 14 | 19377712 | 107283019 | −0.0644 | 0.033 | 1.0444 | 1.0019 | 1 | 1 |
| WCM89_ZLN1_1 | 15 | 20170019 | 102506284 | −0.1184 | −0.1116 | 0.9486 | 0.9026 | 1 | 1 |
| WCM89_ZLN1_1 | 16 | 67604 | 90233622 | −0.1427 | −0.1799 | 1.0739 | 0.6917 | 1 | 1 |
| WCM89_ZLN1_1 | 17 | 131595 | 21201719 | −0.3258 | −0.7782 | 1.0327 | 0.1335 | 1 | 0 |
| WCM89_ZLN1_1 | 17 | 21202237 | 80992927 | −0.0663 | 0.028 | 1.0434 | 0.9957 | 1 | 1 |
| WCM89_ZLN1_1 | 17 | 81006348 | 81083455 | −0.2889 | −0.6428 | NA | NA | NA | NA |
| WCM89_ZLN1_1 | 18 | 49021 | 78005226 | −0.0428 | 0.0884 | 1.0632 | 1.0632 | 1 | 1 |
| WCM89_ZLN1_1 | 19 | 281430 | 59083951 | −0.0873 | −0.0272 | 1.1041 | 0.8585 | 1 | 1 |
| WCM89_ZLN1_1 | 2 | 41614 | 98429177 | −0.0901 | −0.0347 | 1.177 | 0.7754 | 1 | 1 |
| WCM89_ZLN1_1 | 2 | 98430495 | 235404927 | 0.0553 | 0.3251 | 1.2527 | 1.2527 | 1 | 1 |
| WCM89_ZLN1_1 | 2 | 235405215 | 242946616 | −0.0603 | 0.0434 | 1.0811 | 0.98 | 1 | 1 |
| WCM89_ZLN1_1 | 20 | 68351 | 62904848 | −0.0921 | −0.0401 | 0.9882 | 0.957 | 1 | 1 |
| WCM89_ZLN1_1 | 21 | 9907291 | 48084207 | −0.0938 | −0.0445 | 1.2063 | 0.7329 | 1 | 1 |
| WCM89_ZLN1_1 | 22 | 16287488 | 51215157 | −0.1085 | −0.0843 | 0.9844 | 0.902 | 1 | 1 |
| WCM89_ZLN1_1 | 3 | 239398 | 197896720 | −0.0145 | 0.1591 | 1.1358 | 1.0973 | 1 | 1 |
| WCM89_ZLN1_1 | 4 | 59361 | 190426502 | −0.0483 | 0.0742 | 1.0528 | 1.0528 | 1 | 1 |
| WCM89_ZLN1_1 | 5 | 140448 | 180687804 | −0.0333 | 0.1122 | 1.1107 | 1.051 | 1 | 1 |
| WCM89_ZLN1_1 | 6 | 203535 | 170893415 | −0.1467 | −0.1913 | 1.1115 | 0.6401 | 1 | 1 |
| WCM89_ZLN1_1 | 7 | 193451 | 158937416 | −0.0849 | −0.0208 | 1.0145 | 0.9569 | 1 | 1 |
| WCM89_ZLN1_1 | 8 | 190897 | 18257624 | −0.1265 | −0.134 | 0.952 | 0.8706 | 1 | 1 |
| WCM89_ZLN1_1 | 8 | 18257854 | 28409255 | −0.5041 | −1.6184 | 0.5077 | 0.1437 | 1 | 0 |
| WCM89_ZLN1_1 | 8 | 28413362 | 52258494 | −0.2541 | −0.5228 | 0.8078 | 0.5843 | 1 | 1 |
| WCM89_ZLN1_1 | 8 | 52284523 | 67509770 | −0.047 | 0.0777 | 1.0768 | 1.0338 | 1 | 1 |
| WCM89_ZLN1_1 | 8 | 67511377 | 81471724 | 0.2396 | 0.7188 | 2.2391 | 1.0526 | 2 | 1 |
| WCM89_ZLN1_1 | 8 | 81471908 | 142498652 | −0.0506 | 0.0685 | 1.0647 | 1.0325 | 1 | 1 |
| WCM89_ZLN1_1 | 8 | 142504986 | 146279507 | −0.2726 | −0.5857 | 0.9605 | 0.3722 | 1 | 0 |
| WCM89_ZLN1_1 | 9 | 17053 | 271651 | 0.086 | 0.3949 | NA | NA | NA | NA |
| WCM89_ZLN1_1 | 9 | 286593 | 18950895 | −0.2877 | −0.6386 | 1.1244 | 0.1603 | 1 | 0 |
| WCM89_ZLN1_1 | 9 | 19032907 | 141111414 | −0.1063 | −0.0783 | 0.9755 | 0.9188 | 1 | 1 |
| WCM89_ZLN1_1 | X | 2707687 | 154509348 | 0.0051 | 0.2067 | NA | NA | NA | NA |
| WCM9 | 1 | 14907 | 249213964 | 0.0563 | 0.1877 | 1.1389 | 1.1389 | 1 | 1 |
| WCM9 | 10 | 225960 | 135477937 | 0.0359 | 0.1121 | 1.0808 | 1.0808 | 1 | 1 |
| WCM9 | 11 | 193065 | 62301149 | 0.0867 | 0.2953 | 1.2271 | 1.2271 | 1 | 1 |
| WCM9 | 11 | 62301560 | 70332804 | −0.067 | −0.322 | 0.8 | 0.8 | 1 | 1 |
| WCM9 | 11 | 70333178 | 134605906 | 0.0508 | 0.1677 | 1.1233 | 1.1233 | 1 | 1 |
| WCM9 | 12 | 148465 | 133810703 | 0.0077 | 0.0029 | 1.002 | 1.002 | 1 | 1 |
| WCM9 | 13 | 19041965 | 115089411 | −0.0486 | −0.2365 | 0.8488 | 0.8488 | 1 | 1 |
| WCM9 | 14 | 19110239 | 107283269 | 0.006 | −0.0039 | 0.9973 | 0.9973 | 1 | 1 |
| WCM9 | 15 | 20083887 | 102515226 | 0.0612 | 0.2056 | 1.1531 | 1.1531 | 1 | 1 |
| WCM9 | 16 | 64430 | 90168680 | −0.0152 | −0.0909 | 0.9389 | 0.9389 | 1 | 1 |
| WCM9 | 17 | 131595 | 81187925 | 0.0052 | −0.0073 | 0.9949 | 0.9949 | 1 | 1 |
| WCM9 | 18 | 47609 | 78005164 | 0.0021 | −0.0197 | 0.9864 | 0.9864 | 1 | 1 |
| WCM9 | 19 | 105021 | 8992978 | −0.0859 | −0.4139 | 0.7506 | 0.7506 | 1 | 1 |
| WCM9 | 19 | 8993501 | 9204307 | 0.6002 | 1.6014 | 3.1792 | 2.8895 | 3 | 3 |
| WCM9 | 19 | 9204568 | 43093154 | −0.0361 | −0.1807 | 0.8823 | 0.8823 | 1 | 1 |
| WCM9 | 19 | 43093807 | 43858051 | 0.6533 | 1.7051 | 3.4421 | 3.079 | 3 | 3 |
| WCM9 | 19 | 43858556 | 59093696 | −0.0226 | −0.1223 | 0.9187 | 0.9187 | 1 | 1 |
| WCM9 | 2 | 41647 | 243037091 | 0.0192 | 0.0482 | 1.0339 | 1.0339 | 1 | 1 |
| WCM9 | 20 | 68375 | 62959285 | 0.0329 | 0.1008 | 1.0724 | 1.0724 | 1 | 1 |
| WCM9 | 21 | 9590305 | 48084201 | −0.0323 | −0.1643 | 0.8924 | 0.8924 | 1 | 1 |
| WCM9 | 22 | 16159043 | 51223730 | 0.0577 | 0.1928 | 1.143 | 1.143 | 1 | 1 |
| WCM9 | 3 | 239339 | 197896656 | 0.0421 | 0.1356 | 1.0985 | 1.0985 | 1 | 1 |
| WCM9 | 4 | 53446 | 190903980 | −0.0039 | −0.0441 | 0.9699 | 0.9699 | 1 | 1 |
| WCM9 | 5 | 90537 | 140086191 | −9.00E−04 | −0.0318 | 0.9782 | 0.9782 | 1 | 1 |
| WCM9 | 5 | 140166824 | 140812117 | −0.4111 | NA | −0.0064 | −0.0064 | 0 | 0 |
| WCM9 | 5 | 140812420 | 180687821 | 0.032 | 0.0976 | 1.07 | 1.07 | 1 | 1 |
| WCM9 | 6 | 203546 | 170892877 | −0.0041 | −0.0448 | 0.9694 | 0.9694 | 1 | 1 |
| WCM9 | 7 | 193189 | 158937142 | −0.0018 | −0.0357 | 0.9756 | 0.9756 | 1 | 1 |
| WCM9 | 8 | 163527 | 146279384 | −0.0111 | −0.0739 | 0.95 | 0.95 | 1 | 1 |
| WCM9 | 9 | 14822 | 141107545 | 0.0458 | 0.1494 | 1.1091 | 1.1091 | 1 | 1 |
| WCM9 | X | 2700148 | 154507335 | 0.0182 | 0.0443 | NA | NA | NA | NA |
| WCM90 | 1 | 14907 | 1149078 | 0.0145 | 0.0662 | 1.0597 | 1.0343 | 1 | 1 |
| WCM90 | 1 | 1149480 | 1458176 | −0.1143 | −0.366 | 0.8717 | 0.6802 | 1 | 1 |
| WCM90 | 1 | 1458275 | 154744489 | −0.0806 | −0.2437 | 0.8446 | 0.8446 | 1 | 1 |
| WCM90 | 1 | 154744852 | 156956017 | 0.0789 | 0.2529 | 1.1916 | 1.1916 | 1 | 1 |
| WCM90 | 1 | 156957597 | 159785345 | −0.0394 | −0.1036 | 1.4074 | 0.454 | 1 | 0 |
| WCM90 | 1 | 159785453 | 198288709 | −0.1433 | −0.4775 | 1.3096 | 0.1268 | 1 | 0 |
| WCM90 | 1 | 198492696 | 249213964 | 0.0834 | 0.2657 | 1.2022 | 1.2022 | 1 | 1 |
| WCM90 | 10 | 225982 | 135477891 | −0.0763 | −0.2288 | 1.5384 | 0.1683 | 2 | 0 |
| WCM90 | 11 | 193201 | 134605906 | 0.0203 | 0.0837 | 1.0597 | 1.0597 | 1 | 1 |
| WCM90 | 12 | 148576 | 121603118 | 0.0293 | 0.1107 | 1.0797 | 1.0797 | 1 | 1 |
| WCM90 | 12 | 121603856 | 133810703 | −0.1651 | −0.5657 | 0.8969 | 0.4544 | 1 | 0 |
| WCM90 | 13 | 19041994 | 19751794 | −0.0119 | −0.0153 | 1.6746 | 0.3042 | 2 | 0 |
| WCM90 | 13 | 19752392 | 64413947 | −0.0321 | −0.0799 | 1.8767 | 0.0156 | 2 | 0 |
| WCM90 | 13 | 64415622 | 115089411 | −0.1973 | −0.704 | 1.0165 | 0.2113 | 1 | 0 |
| WCM90 | 14 | 19110374 | 107283269 | −0.0626 | −0.1814 | 0.8819 | 0.8819 | 1 | 1 |
| WCM90 | 15 | 20083896 | 102514159 | −0.1002 | −0.3139 | 0.8045 | 0.8045 | 1 | 1 |
| WCM90 | 16 | 67570 | 90260461 | 0.1226 | 0.3716 | 1.2938 | 1.2938 | 1 | 1 |
| WCM90 | 17 | 131595 | 21030347 | −0.2271 | −0.8421 | 0.8449 | 0.2708 | 1 | 0 |
| WCM90 | 17 | 21075425 | 81187855 | 0.0033 | 0.0319 | 1.0224 | 1.0224 | 1 | 1 |
| WCM90 | 18 | 47790 | 78005150 | −0.0149 | −0.025 | 0.9828 | 0.9828 | 1 | 1 |
| WCM90 | 19 | 104937 | 24116208 | −0.0221 | −0.0478 | 0.9674 | 0.9674 | 1 | 1 |
| WCM90 | 19 | 24116307 | 38946014 | 0.2769 | 0.7482 | 1.7482 | 1.6113 | 2 | 2 |
| WCM90 | 19 | 38946182 | 49639242 | −0.2136 | −0.7781 | 0.946 | 0.2202 | 1 | 0 |
| WCM90 | 19 | 49639948 | 57176482 | 0.3253 | 0.8555 | 2.4625 | 1.1563 | 2 | 1 |
| WCM90 | 19 | 57176561 | 59093645 | −0.083 | −0.2522 | 0.8396 | 0.8396 | 1 | 1 |
| WCM90 | 2 | 41670 | 118579684 | 0.0211 | 0.0862 | 1.0616 | 1.0616 | 1 | 1 |
| WCM90 | 2 | 118582099 | 166536072 | −0.2042 | −0.735 | 1.0315 | 0.1701 | 1 | 0 |
| WCM90 | 2 | 166536619 | 243037022 | 0.0763 | 0.2457 | 1.1857 | 1.1857 | 1 | 1 |
| WCM90 | 20 | 68342 | 62934541 | 0.3128 | 0.8283 | 1.7756 | 1.7756 | 2 | 2 |
| WCM90 | 21 | 9590314 | 48084201 | −0.0553 | −0.1567 | 0.8971 | 0.8971 | 1 | 1 |
| WCM90 | 22 | 16159060 | 51219129 | −0.014 | −0.0219 | 0.9849 | 0.9849 | 1 | 1 |
| WCM90 | 3 | 239495 | 197896711 | 0.0379 | 0.136 | 1.0989 | 1.0989 | 1 | 1 |
| WCM90 | 4 | 53469 | 139140567 | 0.0284 | 0.1079 | 1.0777 | 1.0777 | 1 | 1 |
| WCM90 | 4 | 139144360 | 145656089 | 0.3399 | 0.8872 | 2.1595 | 1.5395 | 2 | 2 |
| WCM90 | 4 | 145658878 | 190904471 | 0.0548 | 0.1848 | 1.1367 | 1.1367 | 1 | 1 |
| WCM90 | 5 | 90697 | 21519594 | −0.0243 | −0.0548 | 0.9627 | 0.9627 | 1 | 1 |
| WCM90 | 5 | 21734225 | 39721117 | 0.5234 | 1.2561 | 3.5574 | 1.2195 | 4 | 1 |
| WCM90 | 5 | 40681132 | 83525707 | 0.011 | 0.0554 | 1.0391 | 1.0391 | 1 | 1 |
| WCM90 | 5 | 83549800 | 121767643 | −0.21 | −0.7616 | 1.0486 | 0.131 | 1 | 0 |
| WCM90 | 5 | 121776301 | 145583269 | −0.0037 | 0.0101 | 1.007 | 1.007 | 1 | 1 |
| WCM90 | 5 | 145598650 | 153854557 | 0.1914 | 0.5466 | 1.4786 | 1.4427 | 1 | 1 |
| WCM90 | 5 | 153854872 | 180687776 | 0.0229 | 0.0913 | 1.0653 | 1.0653 | 1 | 1 |
| WCM90 | 6 | 203552 | 170892873 | −0.0775 | −0.233 | 1.5651 | 0.1367 | 2 | 0 |
| WCM90 | 7 | 193254 | 20662947 | 0.2229 | 0.6227 | 1.9393 | 1.1403 | 2 | 1 |
| WCM90 | 7 | 20666193 | 21985462 | −0.2836 | −1.1366 | 0.6793 | 0.2304 | 1 | 0 |
| WCM90 | 7 | 22158277 | 158937011 | 0.1256 | 0.3794 | 1.3145 | 1.2871 | 1 | 1 |
| WCM90 | 8 | 163457 | 34181068 | −0.2508 | −0.9596 | 0.662 | 0.3664 | 1 | 0 |
| WCM90 | 8 | 34181122 | 43415879 | 0.4098 | 1.0332 | 3.223 | 0.8702 | 3 | 1 |
| WCM90 | 8 | 47886271 | 52284471 | −0.0075 | −0.0016 | NA | NA | NA | NA |
| WCM90 | 8 | 52287263 | 65495642 | −0.1854 | −0.6517 | 1.0397 | 0.2333 | 1 | 0 |
| WCM90 | 8 | 65496059 | 146279508 | 0.0011 | 0.0251 | 1.0175 | 1.0175 | 1 | 1 |
| WCM90 | 9 | 14852 | 93976289 | −0.092 | −0.2842 | 0.8212 | 0.8212 | 1 | 1 |
| WCM90 | 9 | 93976659 | 120476591 | 0.0163 | 0.0716 | 1.0509 | 1.0509 | 1 | 1 |
| WCM90 | 9 | 120476758 | 141107582 | −0.1129 | −0.3608 | 0.7817 | 0.7759 | 1 | 1 |
| WCM90 | X | 2700084 | 23899036 | −0.1539 | −0.5198 | NA | NA | NA | NA |
| WCM90 | X | 23928470 | 34150150 | 0.5292 | 1.267 | NA | NA | NA | NA |
| WCM90 | X | 34150365 | 64141724 | 0.0234 | 0.093 | NA | NA | NA | NA |
| WCM90 | X | 64628306 | 69397492 | 1.2896 | 2.4729 | NA | NA | NA | NA |
| WCM90 | X | 69419206 | 154493590 | 0.0489 | 0.1681 | NA | NA | NA | NA |
| WCM90_Z1_1 | 1 | 69649 | 152883866 | −0.2031 | 0.58 | 1.9661 | 1.0236 | 2 | 1 |
| WCM90_Z1_1 | 1 | 152883967 | 153113093 | 1.0344 | 2.038 | NA | NA | NA | NA |
| WCM90_Z1_1 | 1 | 153122578 | 154680706 | −0.2072 | 0.5748 | NA | NA | NA | NA |
| WCM90_Z1_1 | 1 | 154685997 | 155152403 | 0.2568 | 1.1487 | NA | NA | NA | NA |
| WCM90_Z1_1 | 1 | 155154608 | 155629461 | 0.6238 | 1.577 | NA | NA | NA | NA |
| WCM90_Z1_1 | 1 | 155629741 | 157014097 | 0.1281 | 0.9937 | 2.0469 | 1.9356 | 2 | 2 |
| WCM90_Z1_1 | 1 | 157062679 | 158943539 | −0.6247 | 0.0108 | 1.9438 | 0.0712 | 2 | 0 |
| WCM90_Z1_1 | 1 | 158984557 | 159785367 | 0.1674 | 1.0414 | NA | NA | NA | NA |
| WCM90_Z1_1 | 1 | 159796728 | 198288550 | −0.6066 | 0.0366 | 1.9767 | 0.0746 | 2 | 0 |
| WCM90_Z1_1 | 1 | 198492609 | 249212346 | 0.1837 | 1.061 | 2.1116 | 2.0613 | 2 | 2 |
| WCM90_Z1_1 | 10 | 225980 | 135440108 | −0.2166 | 0.5627 | 2.8814 | 0.0726 | 3 | 0 |
| WCM90_Z1_1 | 11 | 193105 | 51412327 | −0.2117 | 0.569 | 1.8812 | 1.0858 | 2 | 1 |
| WCM90_Z1_1 | 11 | 51515549 | 82874879 | 0.0939 | 0.9521 | 1.9347 | 1.9347 | 2 | 2 |
| WCM90_Z1_1 | 11 | 82875318 | 134856563 | −0.206 | 0.5764 | 1.9787 | 1.0035 | 2 | 1 |
| WCM90_Z1_1 | 12 | 148811 | 121660769 | −0.0846 | 0.7309 | 1.9186 | 1.4007 | 2 | 1 |
| WCM90_Z1_1 | 12 | 121660819 | 133254263 | −0.5721 | 0.0852 | 2.0245 | 0.0972 | 2 | 0 |
| WCM90_Z1_1 | 12 | 133256215 | 133810703 | 0.0077 | 0.846 | 2.1248 | 1.4703 | 2 | 1 |
| WCM90_Z1_1 | 13 | 19600475 | 63745832 | −0.1709 | 0.6213 | 2.9781 | 0.0986 | 3 | 0 |
| WCM90_Z1_1 | 13 | 66540556 | 115089411 | −0.5764 | 0.0792 | 2.0439 | 0.0689 | 2 | 0 |
| WCM90_Z1_1 | 14 | 19377692 | 107283183 | −0.0516 | 0.7723 | 1.7315 | 1.6845 | 2 | 2 |
| WCM90_Z1_1 | 15 | 20170150 | 52179801 | −0.5357 | 0.136 | 1.1844 | 1.0133 | 1 | 1 |
| WCM90_Z1_1 | 15 | 52181305 | 102517662 | −0.1803 | 0.6093 | 1.9828 | 1.0683 | 2 | 1 |
| WCM90_Z1_1 | 16 | 67604 | 90233627 | 0.3683 | 1.2807 | 2.9298 | 1.9294 | 3 | 2 |
| WCM90_Z1_1 | 17 | 131595 | 535990 | −0.7536 | −0.1766 | NA | NA | NA | NA |
| WCM90_Z1_1 | 17 | 556546 | 21092139 | −0.6016 | 0.0436 | 1.9836 | 0.0778 | 2 | 0 |
| WCM90_Z1_1 | 17 | 21094369 | 21826154 | −0.6865 | −0.0782 | 1.2417 | 0.6528 | 1 | 1 |
| WCM90_Z1_1 | 17 | 22023461 | 30469681 | 0.0923 | 0.9501 | 1.9721 | 1.8919 | 2 | 2 |
| WCM90_Z1_1 | 17 | 30498088 | 32483536 | −0.626 | 0.009 | NA | NA | NA | NA |
| WCM90_Z1_1 | 17 | 32582389 | 36104713 | 0.0869 | 0.9435 | 2.0032 | 1.8433 | 2 | 2 |
| WCM90_Z1_1 | 17 | 36104803 | 37791860 | −0.6012 | 0.0442 | NA | NA | NA | NA |
| WCM90_Z1_1 | 17 | 37809842 | 81083455 | 0.0884 | 0.9454 | 1.9445 | 1.9068 | 2 | 2 |
| WCM90_Z1_1 | 18 | 48221 | 28611071 | 0.2435 | 1.1328 | 2.2557 | 2.1299 | 2 | 2 |
| WCM90_Z1_1 | 18 | 28612171 | 78005224 | 0.0205 | 0.8619 | 1.8512 | 1.7838 | 2 | 2 |
| WCM90_Z1_1 | 19 | 281498 | 1997375 | −0.5693 | 0.0891 | 1.195 | 0.9324 | 1 | 1 |
| WCM90_Z1_1 | 19 | 2015394 | 4117620 | 0.042 | 0.8884 | 2.6763 | 1.0258 | 3 | 1 |
| WCM90_Z1_1 | 19 | 4153746 | 9091804 | −0.5509 | 0.1149 | 1.1131 | 1.0527 | 1 | 1 |
| WCM90_Z1_1 | 19 | 9203936 | 10169300 | −0.2004 | 0.5836 | 1.8879 | 1.1093 | 2 | 1 |
| WCM90_Z1_1 | 19 | 10169651 | 10362937 | 0.3178 | 1.2211 | NA | NA | NA | NA |
| WCM90_Z1_1 | 19 | 10363335 | 10690481 | −0.5122 | 0.1686 | NA | NA | NA | NA |
| WCM90_Z1_1 | 19 | 10692000 | 17004032 | −0.1998 | 0.5843 | 1.856 | 1.1427 | 2 | 1 |
| WCM90_Z1_1 | 19 | 17004058 | 17015357 | −0.392 | 0.3322 | NA | NA | NA | NA |
| WCM90_Z1_1 | 19 | 17017878 | 23836108 | 0.0475 | 0.8952 | 1.9011 | 1.8187 | 2 | 2 |
| WCM90_Z1_1 | 19 | 23836322 | 35258308 | 0.5398 | 1.4806 | 3.5728 | 2.0083 | 4 | 2 |
| WCM90_Z1_1 | 19 | 35260442 | 36582353 | 0.8601 | 1.8443 | 5.3352 | 1.8464 | 5 | 2 |
| WCM90_Z1_1 | 19 | 36583658 | 38943505 | 0.7184 | 1.6847 | 4.6648 | 1.7647 | 5 | 2 |
| WCM90_Z1_1 | 19 | 38946094 | 49622216 | −0.6582 | −0.0372 | 1.9062 | 0.0429 | 2 | 0 |
| WCM90_Z1_1 | 19 | 49631250 | 57089394 | 0.8602 | 1.8444 | 5.428 | 1.7541 | 5 | 2 |
| WCM90_Z1_1 | 19 | 57089812 | 59083988 | 0.0625 | 0.9136 | 1.9253 | 1.8421 | 2 | 2 |
| WCM90_Z1_1 | 2 | 41613 | 60773427 | 0.1208 | 0.9849 | 1.996 | 1.9623 | 2 | 2 |
| WCM90_Z1_1 | 2 | 60983613 | 61713061 | −0.5858 | 0.066 | NA | NA | NA | NA |
| WCM90_Z1_1 | 2 | 61715313 | 116599804 | 0.0993 | 0.9586 | 1.9435 | 1.9435 | 2 | 2 |
| WCM90_Z1_1 | 2 | 118572380 | 166536237 | −0.5902 | 0.0597 | 1.9983 | 0.0862 | 2 | 0 |
| WCM90_Z1_1 | 2 | 166605407 | 242946607 | 0.1348 | 1.0018 | 2.0099 | 1.9953 | 2 | 2 |
| WCM90_Z1_1 | 20 | 68351 | 33122712 | 0.7226 | 1.6895 | 3.2875 | 3.1633 | 3 | 3 |
| WCM90_Z1_1 | 20 | 33128390 | 49225891 | 0.6155 | 1.5675 | 3.0113 | 2.9164 | 3 | 3 |
| WCM90_Z1_1 | 20 | 49226166 | 62904932 | 0.7753 | 1.749 | 3.9032 | 2.8193 | 4 | 3 |
| WCM90_Z1_1 | 21 | 9907297 | 48084282 | −0.2212 | 0.5568 | 1.8452 | 1.0968 | 2 | 1 |
| WCM90_Z1_1 | 22 | 17063485 | 51219138 | −0.2346 | 0.5394 | 1.8034 | 1.1033 | 2 | 1 |
| WCM90_Z1_1 | 3 | 239347 | 197894719 | 0.097 | 0.9558 | 1.9396 | 1.9396 | 2 | 2 |
| WCM90_Z1_1 | 4 | 59405 | 126412863 | 0.1265 | 0.9918 | 2.4431 | 1.5342 | 2 | 2 |
| WCM90_Z1_1 | 4 | 126428439 | 130032843 | −0.797 | −0.2415 | NA | NA | NA | NA |
| WCM90_Z1_1 | 4 | 133356977 | 190426471 | 0.187 | 1.065 | 2.4602 | 1.7241 | 2 | 2 |
| WCM90_Z1_1 | 5 | 140670 | 26915934 | −0.1983 | 0.5863 | 1.8488 | 1.154 | 2 | 1 |
| WCM90_Z1_1 | 5 | 26916020 | 36301481 | 0.8168 | 1.7957 | 5.184 | 1.7595 | 5 | 2 |
| WCM90_Z1_1 | 5 | 36608583 | 39394363 | 1.2662 | 2.292 | 7.6243 | 2.1705 | 8 | 2 |
| WCM90_Z1_1 | 5 | 40681254 | 50137894 | −0.2894 | 0.4681 | 1.7185 | 1.0481 | 2 | 1 |
| WCM90_Z1_1 | 5 | 50679521 | 82876255 | 0.082 | 0.9376 | 1.9505 | 1.8801 | 2 | 2 |
| WCM90_Z1_1 | 5 | 82937318 | 122139280 | −0.6289 | 0.0048 | 1.937 | 0.0697 | 2 | 0 |
| WCM90_Z1_1 | 5 | 122143973 | 141059972 | 0.0777 | 0.9322 | 1.9619 | 1.8544 | 2 | 2 |
| WCM90_Z1_1 | 5 | 141233942 | 154065351 | 0.3209 | 1.2249 | 2.838 | 1.8367 | 3 | 2 |
| WCM90_Z1_1 | 5 | 154092647 | 170395289 | −0.2083 | 0.5734 | 1.812 | 1.1641 | 2 | 1 |
| WCM90_Z1_1 | 5 | 170395724 | 179071866 | 0.3697 | 1.2824 | 2.7872 | 2.0777 | 3 | 2 |
| WCM90_Z1_1 | 5 | 179078329 | 180687769 | −0.2074 | 0.5746 | 1.8728 | 1.1057 | 2 | 1 |
| WCM90_Z1_1 | 6 | 203588 | 367352 | 0.2031 | 1.0844 | NA | NA | NA | NA |
| WCM90_Z1_1 | 6 | 393323 | 75994343 | −0.183 | 0.6059 | 2.9595 | 0.0844 | 3 | 0 |
| WCM90_Z1_1 | 6 | 76005656 | 105850757 | −0.308 | 0.4437 | 2.6121 | 0.108 | 3 | 0 |
| WCM90_Z1_1 | 6 | 106536118 | 170893408 | −0.0102 | 0.8238 | 3.4806 | 0.0595 | 3 | 0 |
| WCM90_Z1_1 | 7 | 193451 | 15240972 | 0.4051 | 1.3239 | 3.946 | 1.0609 | 4 | 1 |
| WCM90_Z1_1 | 7 | 15346160 | 20449577 | 0.1963 | 1.0762 | NA | NA | NA | NA |
| WCM90_Z1_1 | 7 | 20662957 | 22162118 | −1.0665 | −0.6696 | NA | NA | NA | NA |
| WCM90_Z1_1 | 7 | 22165242 | 38783060 | 0.233 | 1.1202 | 3.1263 | 1.2214 | 3 | 1 |
| WCM90_Z1_1 | 7 | 38785187 | 38869884 | −0.9957 | −0.5524 | NA | NA | NA | NA |
| WCM90_Z1_1 | 7 | 38870816 | 55985759 | 0.1901 | 1.0688 | 3.1511 | 1.0442 | 3 | 1 |
| WCM90_Z1_1 | 7 | 55990881 | 117365164 | 0.4772 | 1.4081 | 2.7001 | 2.6076 | 3 | 3 |
| WCM90_Z1_1 | 7 | 117368316 | 158937400 | 0.1437 | 1.0127 | 2.0568 | 1.9786 | 2 | 2 |
| WCM90_Z1_1 | 8 | 190906 | 33454845 | −0.9825 | −0.531 | 1.267 | 0.1172 | 1 | 0 |
| WCM90_Z1_1 | 8 | 35406847 | 43211967 | 0.9549 | 1.9499 | 6.4432 | 1.284 | 6 | 1 |
| WCM90_Z1_1 | 8 | 47752739 | 50449716 | −0.0271 | 0.8028 | NA | NA | NA | NA |
| WCM90_Z1_1 | 8 | 51306797 | 65528834 | −0.4915 | 0.1971 | 2.2394 | 0.0534 | 2 | 0 |
| WCM90_Z1_1 | 8 | 65536969 | 146279510 | −0.025 | 0.8054 | 2.2264 | 1.2689 | 2 | 1 |
| WCM90_Z1_1 | 9 | 17075 | 141111414 | −0.2245 | 0.5525 | 1.8495 | 1.0838 | 2 | 1 |
| WCM90_Z1_1 | X | 2707709 | 8993583 | 0.0452 | 0.8923 | NA | NA | NA | NA |
| WCM90_Z1_1 | X | 8996004 | 23892569 | −0.5551 | 0.109 | NA | NA | NA | NA |
| WCM90_Z1_1 | X | 23897107 | 29417325 | 0.4638 | 1.3924 | NA | NA | NA | NA |
| WCM90_Z1_1 | X | 29592467 | 33229421 | 1.4519 | 2.4929 | NA | NA | NA | NA |
| WCM90_Z1_1 | X | 34148186 | 64196239 | 0.0418 | 0.8881 | NA | NA | NA | NA |
| WCM90_Z1_1 | X | 64708914 | 69397495 | 2.2606 | 3.3479 | NA | NA | NA | NA |
| WCM90_Z1_1 | X | 69419206 | 154509348 | 0.0568 | 0.9066 | NA | NA | NA | NA |
| WCM90_Z19_1 | 1 | 69634 | 157014097 | 0.0018 | 0.0292 | 1.0372 | 1.0037 | 1 | 1 |
| WCM90_Z19_1 | 1 | 157062614 | 198700746 | −0.2376 | −0.9781 | 1.0616 | −0.0463 | 1 | 0 |
| WCM90_Z19_1 | 1 | 198701436 | 249212341 | −0.0066 | 0.0012 | 1.0009 | 1.0009 | 1 | 1 |
| WCM90_Z19_1 | 10 | 225979 | 135440080 | −0.0044 | 0.0086 | 2.0311 | −0.0192 | 2 | 0 |
| WCM90_Z19_1 | 11 | 193097 | 134856626 | −0.0958 | −0.3199 | 1.124 | 0.4783 | 1 | 0 |
| WCM90_Z19_1 | 12 | 148675 | 121659917 | 0.0085 | 0.0511 | 1.0562 | 1.0158 | 1 | 1 |
| WCM90_Z19_1 | 12 | 121660769 | 133254282 | −0.2072 | −0.816 | 1.1193 | 0.0168 | 1 | 0 |
| WCM90_Z19_1 | 12 | 133256218 | 133810703 | 0.0705 | 0.2435 | 1.3668 | 1.001 | 1 | 1 |
| WCM90_Z19_1 | 13 | 19600515 | 63745832 | −0.1018 | −0.3436 | 1.7401 | −0.164 | 2 | 0 |
| WCM90_Z19_1 | 13 | 66540556 | 115089411 | −0.2587 | −1.1003 | 0.8948 | 0.0381 | 1 | 0 |
| WCM90_Z19_1 | 14 | 19377727 | 107283091 | −0.0015 | 0.0182 | 1.0361 | 0.9893 | 1 | 1 |
| WCM90_Z19_1 | 15 | 20170150 | 102517678 | −0.0556 | −0.1687 | 0.9251 | 0.8542 | 1 | 1 |
| WCM90_Z19_1 | 16 | 67604 | 90233626 | 0.1494 | 0.4663 | 1.4299 | 1.3333 | 1 | 1 |
| WCM90_Z19_1 | 17 | 131595 | 25628910 | −0.2045 | −0.8019 | 1.0649 | 0.0823 | 1 | 0 |
| WCM90_Z19_1 | 17 | 25630569 | 36099545 | 0.0321 | 0.1264 | 1.1498 | 1.0333 | 1 | 1 |
| WCM90_Z19_1 | 17 | 36104698 | 37785430 | −0.2331 | −0.9532 | NA | NA | NA | NA |
| WCM90_Z19_1 | 17 | 37785802 | 81083419 | 0.0407 | 0.1532 | 1.1465 | 1.0776 | 1 | 1 |
| WCM90_Z19_1 | 18 | 48235 | 2954511 | −0.1735 | −0.6512 | 1.3199 | −0.0464 | 1 | 0 |
| WCM90_Z19_1 | 18 | 2960708 | 33555048 | 0.0588 | 0.2086 | 1.2253 | 1.0858 | 1 | 1 |
| WCM90_Z19_1 | 18 | 33557534 | 78005224 | −0.1356 | −0.4825 | 1.2767 | 0.1547 | 1 | 0 |
| WCM90_Z19_1 | 19 | 281534 | 24309552 | −0.0137 | −0.0225 | 1.0177 | 0.9514 | 1 | 1 |
| WCM90_Z19_1 | 19 | 24309782 | 38943459 | 0.291 | 0.8194 | 2.7114 | 0.8181 | 3 | 1 |
| WCM90_Z19_1 | 19 | 38945965 | 49622280 | −0.2785 | −1.2236 | 0.8148 | 0.0416 | 1 | 0 |
| WCM90_Z19_1 | 19 | 49631346 | 57036381 | 0.3691 | 0.9947 | 3.1996 | 0.7857 | 3 | 1 |
| WCM90_Z19_1 | 19 | 57036653 | 59083968 | −0.0217 | −0.0498 | 1.0222 | 0.91 | 1 | 1 |
| WCM90_Z19_1 | 2 | 41613 | 114715011 | 0.0067 | 0.0451 | 1.0486 | 1.015 | 1 | 1 |
| WCM90_Z19_1 | 2 | 115200374 | 166771711 | −0.2426 | −1.0063 | 1.0442 | −0.0486 | 1 | 0 |
| WCM90_Z19_1 | 2 | 166773971 | 183902737 | −0.0055 | 0.0049 | 1.0034 | 1.0034 | 1 | 1 |
| WCM90_Z19_1 | 2 | 183943812 | 242813150 | 0.1332 | 0.4225 | 1.388 | 1.2925 | 1 | 1 |
| WCM90_Z19_1 | 2 | 242814009 | 242946607 | −0.1962 | −0.7604 | NA | NA | NA | NA |
| WCM90_Z19_1 | 20 | 68406 | 62904861 | 0.3788 | 1.0158 | 2.022 | 2.022 | 2 | 2 |
| WCM90_Z19_1 | 21 | 9907313 | 48084282 | −0.0061 | 0.0029 | 1.0621 | 0.942 | 1 | 1 |
| WCM90_Z19_1 | 22 | 17063496 | 51219138 | −0.0775 | −0.2498 | 0.8728 | 0.8093 | 1 | 1 |
| WCM90_Z19_1 | 3 | 239347 | 197894726 | 0.0092 | 0.0533 | 1.0376 | 1.0376 | 1 | 1 |
| WCM90_Z19_1 | 4 | 59404 | 190426459 | −0.0155 | −0.0286 | 1.0135 | 0.9472 | 1 | 1 |
| WCM90_Z19_1 | 5 | 140579 | 83362279 | −1.00E−04 | 0.023 | 1.0395 | 0.9926 | 1 | 1 |
| WCM90_Z19_1 | 5 | 83402477 | 122426238 | −0.2786 | −1.2243 | 0.9471 | −0.0911 | 1 | 0 |
| WCM90_Z19_1 | 5 | 122435582 | 145522987 | 0.0211 | 0.0916 | 1.1369 | 0.9942 | 1 | 1 |
| WCM90_Z19_1 | 5 | 145524050 | 154092578 | 0.2186 | 0.6456 | 1.7631 | 1.3656 | 2 | 1 |
| WCM90_Z19_1 | 5 | 154135401 | 170395343 | 0.0183 | 0.0825 | 1.1742 | 0.9435 | 1 | 1 |
| WCM90_Z19_1 | 5 | 170395731 | 179229105 | 0.2476 | 0.7167 | 2.0212 | 1.2656 | 2 | 1 |
| WCM90_Z19_1 | 5 | 179248027 | 180687776 | −0.0166 | −0.0323 | 1.0573 | 0.8984 | 1 | 1 |
| WCM90_Z19_1 | 6 | 203588 | 170893438 | −0.118 | −0.4089 | 1.5403 | −0.0339 | 2 | 0 |
| WCM90_Z19_1 | 7 | 193245 | 11441553 | 0.1959 | 0.5883 | 1.6224 | 1.3845 | 2 | 1 |
| WCM90_Z19_1 | 7 | 11446060 | 40027425 | 0.0193 | 0.0859 | 1.1496 | 0.9731 | 1 | 1 |
| WCM90_Z19_1 | 7 | 40027611 | 103824602 | 0.202 | 0.6036 | 2.0628 | 0.9763 | 2 | 1 |
| WCM90_Z19_1 | 7 | 103828827 | 126249446 | −0.0283 | −0.0721 | 1.0859 | 0.8166 | 1 | 1 |
| WCM90_Z19_1 | 7 | 126341876 | 129690318 | 0.2201 | 0.6492 | 1.8492 | 1.2874 | 2 | 1 |
| WCM90_Z19_1 | 7 | 129691167 | 137597573 | −0.0217 | −0.0496 | 1.1411 | 0.7913 | 1 | 1 |
| WCM90_Z19_1 | 7 | 137600739 | 158937400 | 0.2065 | 0.6152 | 2.0806 | 0.9829 | 2 | 1 |
| WCM90_Z19_1 | 8 | 190907 | 51314769 | 0.0314 | 0.1241 | 1.1514 | 1.0282 | 1 | 1 |
| WCM90_Z19_1 | 8 | 51351139 | 65536982 | −0.2489 | −1.0426 | 1.0041 | −0.0332 | 1 | 0 |
| WCM90_Z19_1 | 8 | 66439766 | 146279507 | 0.0233 | 0.0987 | 1.0708 | 1.0708 | 1 | 1 |
| WCM90_Z19_1 | 9 | 17086 | 1051973 | −0.3758 | −2.0109 | 0.4082 | 0.0881 | 0 | 0 |
| WCM90_Z19_1 | 9 | 1053739 | 6814636 | −0.1192 | −0.4139 | 0.9572 | 0.544 | 1 | 1 |
| WCM90_Z19_1 | 9 | 6849614 | 28863675 | −0.3277 | −1.5751 | 0.5064 | 0.1648 | 1 | 0 |
| WCM90_Z19_1 | 9 | 32384732 | 38595673 | −0.1149 | −0.3964 | 0.834 | 0.6855 | 1 | 1 |
| WCM90_Z19_1 | 9 | 38596201 | 79118585 | −0.3913 | −2.1812 | 0.4989 | −0.0579 | 0 | 0 |
| WCM90_Z19_1 | 9 | 79234286 | 110249505 | −0.1509 | −0.5487 | 0.9702 | 0.397 | 1 | 0 |
| WCM90_Z19_1 | 9 | 110249584 | 122004387 | −0.3756 | −2.0086 | 0.3156 | 0.1814 | 0 | 0 |
| WCM90_Z19_1 | 9 | 122011331 | 134889825 | −0.0891 | −0.2941 | 0.8974 | 0.7337 | 1 | 1 |
| WCM90_Z19_1 | 9 | 134949107 | 141111414 | −0.3234 | −1.5411 | 0.4427 | 0.2446 | 0 | 0 |
| WCM90_Z19_1 | X | 2707711 | 154509348 | −0.0358 | −0.0985 | NA | NA | NA | NA |
| WCM90_Z21_1 | 1 | 69635 | 152883816 | −0.3165 | −0.3909 | 1.0094 | 0.516 | 1 | 1 |
| WCM90_Z21_1 | 1 | 152883957 | 153113025 | 0.9887 | 1.1283 | NA | NA | NA | NA |
| WCM90_Z21_1 | 1 | 153122393 | 154698473 | −0.318 | −0.3927 | NA | NA | NA | NA |
| WCM90_Z21_1 | 1 | 154728827 | 155629535 | 0.4515 | 0.5288 | 1.657 | 1.2286 | 2 | 1 |
| WCM90_Z21_1 | 1 | 155629762 | 156628006 | −0.0036 | −0.0044 | NA | NA | NA | NA |
| WCM90_Z21_1 | 1 | 156628475 | 157014097 | 0.2968 | 0.3507 | NA | NA | NA | NA |
| WCM90_Z21_1 | 1 | 157062657 | 158943505 | −0.6737 | −0.8612 | 1.0739 | 0.0271 | 1 | 0 |
| WCM90_Z21_1 | 1 | 158984681 | 159785346 | 0.3159 | 0.3728 | NA | NA | NA | NA |
| WCM90_Z21_1 | 1 | 159796693 | 198288563 | −0.7477 | −0.9638 | 1.0422 | −0.0168 | 1 | 0 |
| WCM90_Z21_1 | 1 | 198492616 | 249212434 | 0.2689 | 0.3183 | 1.4602 | 1.0334 | 1 | 1 |
| WCM90_Z21_1 | 10 | 225981 | 135440081 | −0.3268 | −0.4039 | 1.5214 | −0.0098 | 2 | 0 |
| WCM90_Z21_1 | 11 | 193097 | 134856556 | −0.0039 | −0.0047 | 1.004 | 0.9896 | 1 | 1 |
| WCM90_Z21_1 | 12 | 148675 | 34179683 | −0.02 | −0.0242 | 0.9858 | 0.981 | 1 | 1 |
| WCM90_Z21_1 | 12 | 34182559 | 41582590 | 0.4564 | 0.5343 | 1.893 | 1.0036 | 2 | 1 |
| WCM90_Z21_1 | 12 | 41582881 | 46756340 | 0.054 | 0.0649 | 1.0998 | 0.9922 | 1 | 1 |
| WCM90_Z21_1 | 12 | 46756890 | 48240146 | 0.4203 | 0.4932 | NA | NA | NA | NA |
| WCM90_Z21_1 | 12 | 48240461 | 50500104 | −0.0055 | −0.0067 | 1.0482 | 0.9426 | 1 | 1 |
| WCM90_Z21_1 | 12 | 50500746 | 52470833 | 0.4357 | 0.5107 | 1.9413 | 0.9082 | 2 | 1 |
| WCM90_Z21_1 | 12 | 52565235 | 52914037 | 0.8261 | 0.9495 | 2.7385 | 1.1237 | 3 | 1 |
| WCM90_Z21_1 | 12 | 52938364 | 54350229 | 0.4906 | 0.5734 | 1.9975 | 0.9784 | 2 | 1 |
| WCM90_Z21_1 | 12 | 54367154 | 57843532 | −0.0289 | −0.0349 | 0.995 | 0.9572 | 1 | 1 |
| WCM90_Z21_1 | 12 | 57843737 | 71077926 | 0.5331 | 0.6217 | 2.0861 | 0.9912 | 2 | 1 |
| WCM90_Z21_1 | 12 | 71078517 | 85285862 | −0.0343 | −0.0414 | 1.0188 | 0.9247 | 1 | 1 |
| WCM90_Z21_1 | 12 | 85408291 | 96422886 | 0.5256 | 0.6131 | 2.1955 | 0.8635 | 2 | 1 |
| WCM90_Z21_1 | 12 | 96429295 | 98850629 | −0.0422 | −0.051 | NA | NA | NA | NA |
| WCM90_Z21_1 | 12 | 98880948 | 111052034 | 0.4927 | 0.5758 | 2.0449 | 0.9361 | 2 | 1 |
| WCM90_Z21_1 | 12 | 111057639 | 112844686 | 0.8018 | 0.9226 | NA | NA | NA | NA |
| WCM90_Z21_1 | 12 | 112846128 | 118852236 | 0.4366 | 0.5117 | 1.8399 | 1.0116 | 2 | 1 |
| WCM90_Z21_1 | 12 | 119419708 | 121660770 | −0.0206 | −0.0248 | 1.0221 | 0.9438 | 1 | 1 |
| WCM90_Z21_1 | 12 | 121660838 | 123110771 | −0.7222 | −0.9282 | 1.0616 | −0.0106 | 1 | 0 |
| WCM90_Z21_1 | 12 | 123186730 | 133252758 | 0.0079 | 0.0096 | 2.0073 | 0.006 | 2 | 0 |
| WCM90_Z21_1 | 12 | 133253159 | 133810703 | 0.5071 | 0.5921 | 2.0828 | 0.9321 | 2 | 1 |
| WCM90_Z21_1 | 13 | 19600475 | 63745832 | −0.2965 | −0.3656 | 1.5335 | 0.0188 | 2 | 0 |
| WCM90_Z21_1 | 13 | 66540556 | 115089411 | −0.7471 | −0.963 | 1.0262 | −2.00E−04 | 1 | 0 |
| WCM90_Z21_1 | 14 | 19377727 | 107283197 | −0.1298 | −0.1579 | 0.9822 | 0.8104 | 1 | 1 |
| WCM90_Z21_1 | 15 | 20170150 | 102517666 | −0.3218 | −0.3976 | 1.0088 | 0.5095 | 1 | 1 |
| WCM90_Z21_1 | 16 | 67604 | 1004554 | 0.4095 | 0.4807 | 1.8195 | 0.9714 | 2 | 1 |
| WCM90_Z21_1 | 16 | 1004658 | 3724437 | 0.2111 | 0.2508 | 1.3823 | 0.9974 | 1 | 1 |
| WCM90_Z21_1 | 16 | 3725395 | 50138915 | 0.3968 | 0.4662 | 1.7637 | 0.9991 | 2 | 1 |
| WCM90_Z21_1 | 16 | 50186890 | 90233622 | 0.1028 | 0.1229 | 1.2429 | 0.935 | 1 | 1 |
| WCM90_Z21_1 | 17 | 131595 | 21208370 | −0.7526 | −0.9706 | 1.0188 | 0.0019 | 1 | 0 |
| WCM90_Z21_1 | 17 | 21215537 | 30469651 | 0.0176 | 0.0211 | 1.0465 | 0.983 | 1 | 1 |
| WCM90_Z21_1 | 17 | 30498106 | 32483427 | −0.6863 | −0.8786 | NA | NA | NA | NA |
| WCM90_Z21_1 | 17 | 32582389 | 36104728 | 0.0224 | 0.0269 | 1.0947 | 0.943 | 1 | 1 |
| WCM90_Z21_1 | 17 | 36104800 | 37809839 | −0.7965 | −1.0325 | NA | NA | NA | NA |
| WCM90_Z21_1 | 17 | 37813285 | 81083419 | 0.0159 | 0.0191 | 1.0341 | 0.9926 | 1 | 1 |
| WCM90_Z21_1 | 18 | 48206 | 78005199 | 0.0086 | 0.0103 | 1.0278 | 0.9866 | 1 | 1 |
| WCM90_Z21_1 | 19 | 281405 | 1997366 | −0.2703 | −0.3325 | 1.0479 | 0.5404 | 1 | 1 |
| WCM90_Z21_1 | 19 | 2015393 | 4117566 | 0.2506 | 0.297 | 1.3963 | 1.0608 | 1 | 1 |
| WCM90_Z21_1 | 19 | 4153748 | 9091814 | −0.3252 | −0.402 | 0.9915 | 0.5222 | 1 | 1 |
| WCM90_Z21_1 | 19 | 9204111 | 10166375 | 0.0478 | 0.0574 | 1.0717 | 1.0094 | 1 | 1 |
| WCM90_Z21_1 | 19 | 10169265 | 10362938 | 0.5585 | 0.6504 | NA | NA | NA | NA |
| WCM90_Z21_1 | 19 | 10363286 | 10394239 | 0.0565 | 0.0678 | NA | NA | NA | NA |
| WCM90_Z21_1 | 19 | 10394452 | 10685607 | −0.359 | −0.445 | NA | NA | NA | NA |
| WCM90_Z21_1 | 19 | 10687990 | 24310734 | −0.0013 | −0.0015 | 1.0158 | 0.9821 | 1 | 1 |
| WCM90_Z21_1 | 19 | 29567134 | 30506455 | 0.8262 | 0.9496 | NA | NA | NA | NA |
| WCM90_Z21_1 | 19 | 30934584 | 35256531 | 0.5011 | 0.5853 | 1.9675 | 1.0331 | 2 | 1 |
| WCM90_Z21_1 | 19 | 35258264 | 36582353 | 0.8431 | 0.9683 | 2.9788 | 0.9342 | 3 | 1 |
| WCM90_Z21_1 | 19 | 36583651 | 38056745 | 0.5212 | 0.6081 | 1.915 | 1.1335 | 2 | 1 |
| WCM90_Z21_1 | 19 | 38057059 | 38943512 | 0.8629 | 0.9902 | 2.9681 | 1.0047 | 3 | 1 |
| WCM90_Z21_1 | 19 | 38946112 | 49631306 | −0.7833 | −1.0139 | 0.9999 | −0.0095 | 1 | 0 |
| WCM90_Z21_1 | 19 | 49631653 | 57132937 | 0.8682 | 0.996 | 2.982 | 1.007 | 3 | 1 |
| WCM90_Z21_1 | 19 | 57133421 | 59083923 | 0.0064 | 0.0077 | 1.0054 | 1.0054 | 1 | 1 |
| WCM90_Z21_1 | 2 | 41620 | 60773167 | 0.0261 | 0.0314 | 1.0318 | 1.0122 | 1 | 1 |
| WCM90_Z21_1 | 2 | 60773434 | 61715377 | −0.7316 | −0.9413 | NA | NA | NA | NA |
| WCM90_Z21_1 | 2 | 61715763 | 116599847 | 0.0191 | 0.023 | 1.0307 | 1.0014 | 1 | 1 |
| WCM90_Z21_1 | 2 | 118572371 | 166605395 | −0.7438 | −0.9583 | 1.0385 | −0.0091 | 1 | 0 |
| WCM90_Z21_1 | 2 | 166606327 | 242946604 | 0.0337 | 0.0405 | 1.0383 | 1.0186 | 1 | 1 |
| WCM90_Z21_1 | 20 | 68406 | 37356764 | 0.8223 | 0.9453 | 1.9256 | 1.9256 | 2 | 2 |
| WCM90_Z21_1 | 20 | 37356993 | 48894078 | 0.6886 | 0.7966 | 1.9408 | 1.5331 | 2 | 2 |
| WCM90_Z21_1 | 20 | 48894756 | 49199249 | 0.8618 | 0.989 | NA | NA | NA | NA |
| WCM90_Z21_1 | 20 | 49202351 | 62904845 | 1.007 | 1.1483 | 2.294 | 2.139 | 2 | 2 |
| WCM90_Z21_1 | 21 | 9907301 | 48084210 | −0.0285 | −0.0344 | 0.9906 | 0.9623 | 1 | 1 |
| WCM90_Z21_1 | 22 | 17063496 | 51219138 | −0.1132 | −0.1376 | 0.9247 | 0.8935 | 1 | 1 |
| WCM90_Z21_1 | 3 | 239347 | 85984969 | 0.0155 | 0.0187 | 1.0203 | 1.0057 | 1 | 1 |
| WCM90_Z21_1 | 3 | 86010733 | 197894726 | 0.2486 | 0.2947 | 1.4585 | 0.9947 | 1 | 1 |
| WCM90_Z21_1 | 4 | 59331 | 126428439 | 0.0146 | 0.0176 | 1.0123 | 1.0123 | 1 | 1 |
| WCM90_Z21_1 | 4 | 128544639 | 130032843 | −0.7809 | −1.0105 | NA | NA | NA | NA |
| WCM90_Z21_1 | 4 | 133357010 | 190426459 | −0.0017 | −0.0021 | 1.0286 | 0.9685 | 1 | 1 |
| WCM90_Z21_1 | 5 | 140560 | 26915966 | −0.3162 | −0.3905 | 0.9477 | 0.578 | 1 | 1 |
| WCM90_Z21_1 | 5 | 26916024 | 35702306 | 0.8106 | 0.9323 | 2.8191 | 0.9976 | 3 | 1 |
| WCM90_Z21_1 | 5 | 35704684 | 39394363 | 1.2252 | 1.3851 | 4.0934 | 1.1303 | 4 | 1 |
| WCM90_Z21_1 | 5 | 40681254 | 50137815 | −0.3649 | −0.4525 | 0.9179 | 0.5437 | 1 | 1 |
| WCM90_Z21_1 | 5 | 50679527 | 82849325 | 0.0155 | 0.0186 | 1.0313 | 0.9947 | 1 | 1 |
| WCM90_Z21_1 | 5 | 82850775 | 121799209 | −0.7467 | −0.9623 | 1.0396 | −0.0131 | 1 | 0 |
| WCM90_Z21_1 | 5 | 122131040 | 141059944 | 0.0196 | 0.0236 | 1.058 | 0.975 | 1 | 1 |
| WCM90_Z21_1 | 5 | 141233873 | 154065351 | 0.2714 | 0.3212 | 1.4414 | 1.0575 | 1 | 1 |
| WCM90_Z21_1 | 5 | 154092634 | 170395475 | −0.3084 | −0.3806 | 1.0104 | 0.5259 | 1 | 1 |
| WCM90_Z21_1 | 5 | 170395724 | 179079306 | 0.2957 | 0.3495 | 1.5107 | 1.0375 | 2 | 1 |
| WCM90_Z21_1 | 5 | 179105658 | 180687773 | −0.2876 | −0.3542 | 1.0113 | 0.5533 | 1 | 1 |
| WCM90_Z21_1 | 6 | 203542 | 74533254 | −0.2958 | −0.3647 | 1.55 | 0.0032 | 2 | 0 |
| WCM90_Z21_1 | 6 | 74537868 | 105850763 | −0.7 | −0.8975 | 1.0767 | −0.0031 | 1 | 0 |
| WCM90_Z21_1 | 6 | 106536255 | 170893411 | −0.2969 | −0.366 | 1.5488 | 0.0031 | 2 | 0 |
| WCM90_Z21_1 | 7 | 193451 | 20449251 | 0.5614 | 0.6537 | 2.1474 | 0.9989 | 2 | 1 |
| WCM90_Z21_1 | 7 | 20449551 | 21985424 | −0.7559 | −0.9752 | NA | NA | NA | NA |
| WCM90_Z21_1 | 7 | 22162085 | 38783051 | 0.3823 | 0.4495 | 1.7079 | 1.0232 | 2 | 1 |
| WCM90_Z21_1 | 7 | 38785188 | 38860819 | −0.5893 | −0.7466 | NA | NA | NA | NA |
| WCM90_Z21_1 | 7 | 38869897 | 51261144 | 0.4134 | 0.4852 | 1.756 | 1.0436 | 2 | 1 |
| WCM90_Z21_1 | 7 | 51287470 | 54636754 | 0.0511 | 0.0613 | NA | NA | NA | NA |
| WCM90_Z21_1 | 7 | 54820103 | 84751118 | 0.1773 | 0.2111 | 1.3823 | 0.9327 | 1 | 1 |
| WCM90_Z21_1 | 7 | 86274171 | 86567465 | −0.7368 | −0.9485 | NA | NA | NA | NA |
| WCM90_Z21_1 | 7 | 86569426 | 117876925 | 0.1633 | 0.1946 | 1.3373 | 0.9515 | 1 | 1 |
| WCM90_Z21_1 | 7 | 117879999 | 158937400 | −0.1077 | −0.1308 | 0.9636 | 0.863 | 1 | 1 |
| WCM90_Z21_1 | 8 | 190900 | 33455009 | −0.7318 | −0.9416 | 1.0161 | 0.0252 | 1 | 0 |
| WCM90_Z21_1 | 8 | 35407005 | 47752739 | 1.266 | 1.4291 | 4.3472 | 1.0382 | 4 | 1 |
| WCM90_Z21_1 | 8 | 47753945 | 50449716 | 0.2616 | 0.3097 | NA | NA | NA | NA |
| WCM90_Z21_1 | 8 | 51306825 | 65537003 | −0.3433 | −0.425 | 1.4485 | 0.0411 | 1 | 0 |
| WCM90_Z21_1 | 8 | 66439725 | 146279510 | 0.2773 | 0.3281 | 1.4554 | 1.0553 | 1 | 1 |
| WCM90_Z21_1 | 9 | 17107 | 113228268 | −0.3175 | −0.3921 | 0.995 | 0.529 | 1 | 1 |
| WCM90_Z21_1 | 9 | 113231236 | 131088114 | −0.0087 | −0.0104 | 1.0036 | 0.982 | 1 | 1 |
| WCM90_Z21_1 | 9 | 131094519 | 141111414 | −0.718 | −0.9224 | 1.0243 | 0.031 | 1 | 0 |
| WCM90_Z21_1 | X | 2707700 | 9001009 | −0.0987 | −0.1198 | NA | NA | NA | NA |
| WCM90_Z21_1 | X | 9621705 | 23899036 | −0.7767 | −1.0046 | NA | NA | NA | NA |
| WCM90_Z21_1 | X | 23933912 | 29417279 | 0.4557 | 0.5336 | NA | NA | NA | NA |
| WCM90_Z21_1 | X | 29592467 | 33229421 | 2.1223 | 2.3336 | NA | NA | NA | NA |
| WCM90_Z21_1 | X | 34148248 | 64196239 | −0.0306 | −0.0369 | NA | NA | NA | NA |
| WCM90_Z21_1 | X | 64708855 | 69397504 | 3.2113 | 3.4534 | NA | NA | NA | NA |
| WCM90_Z21_1 | X | 69419227 | 154509348 | −0.0265 | −0.032 | NA | NA | NA | NA |
| WCM90_Z25_1 | 1 | 69569 | 157014089 | −0.088 | NA | NA | NA | NA | NA |
| WCM90_Z25_1 | 1 | 157062660 | 198262160 | −0.2206 | NA | NA | NA | NA | NA |
| WCM90_Z25_1 | 1 | 198288614 | 249212373 | −0.0849 | NA | NA | NA | NA | NA |
| WCM90_Z25_1 | 10 | 225989 | 135440105 | −0.0347 | NA | NA | NA | NA | NA |
| WCM90_Z25_1 | 11 | 193102 | 134856592 | −0.0836 | NA | NA | NA | NA | NA |
| WCM90_Z25_1 | 12 | 148644 | 121666868 | 0.0269 | NA | NA | NA | NA | NA |
| WCM90_Z25_1 | 12 | 121670294 | 133254258 | −0.1948 | NA | NA | NA | NA | NA |
| WCM90_Z25_1 | 12 | 133256230 | 133810703 | 0.0154 | NA | NA | NA | NA | NA |
| WCM90_Z25_1 | 13 | 19600475 | 61988094 | −0.0994 | NA | NA | NA | NA | NA |
| WCM90_Z25_1 | 13 | 61989235 | 115028480 | −0.2218 | NA | NA | NA | NA | NA |
| WCM90_Z25_1 | 13 | 115030714 | 115089411 | −0.1064 | NA | NA | NA | NA | NA |
| WCM90_Z25_1 | 14 | 19377733 | 107283096 | 0.0227 | NA | NA | NA | NA | NA |
| WCM90_Z25_1 | 15 | 20170150 | 102517667 | −0.0177 | NA | NA | NA | NA | NA |
| WCM90_Z25_1 | 16 | 67604 | 90233622 | 0.1282 | NA | NA | NA | NA | NA |
| WCM90_Z25_1 | 17 | 131595 | 25637146 | −0.1972 | NA | NA | NA | NA | NA |
| WCM90_Z25_1 | 17 | 25639374 | 36358124 | 0.0131 | NA | NA | NA | NA | NA |
| WCM90_Z25_1 | 17 | 36358956 | 37785473 | −0.2225 | NA | NA | NA | NA | NA |
| WCM90_Z25_1 | 17 | 37785806 | 81083419 | 0.0372 | NA | NA | NA | NA | NA |
| WCM90_Z25_1 | 18 | 48241 | 2960731 | −0.1678 | NA | NA | NA | NA | NA |
| WCM90_Z25_1 | 18 | 2982674 | 33289660 | 0.0462 | NA | NA | NA | NA | NA |
| WCM90_Z25_1 | 18 | 33484800 | 78005210 | −0.1224 | NA | NA | NA | NA | NA |
| WCM90_Z25_1 | 19 | 281403 | 24309680 | −0.0722 | NA | NA | NA | NA | NA |
| WCM90_Z25_1 | 19 | 24310032 | 38943505 | 0.2351 | NA | NA | NA | NA | NA |
| WCM90_Z25_1 | 19 | 38945988 | 49631284 | −0.3473 | NA | NA | NA | NA | NA |
| WCM90_Z25_1 | 19 | 49631641 | 57134012 | 0.3238 | NA | NA | NA | NA | NA |
| WCM90_Z25_1 | 19 | 57157110 | 59083985 | −0.0744 | NA | NA | NA | NA | NA |
| WCM90_Z25_1 | 2 | 41620 | 114709329 | 0.0251 | NA | NA | NA | NA | NA |
| WCM90_Z25_1 | 2 | 114713215 | 166611485 | −0.2117 | NA | NA | NA | NA | NA |
| WCM90_Z25_1 | 2 | 166613648 | 187625926 | 0.0237 | NA | NA | NA | NA | NA |
| WCM90_Z25_1 | 2 | 187626338 | 242946607 | 0.1441 | NA | NA | NA | NA | NA |
| WCM90_Z25_1 | 20 | 68373 | 62904925 | 0.2777 | NA | NA | NA | NA | NA |
| WCM90_Z25_1 | 21 | 9907280 | 48084210 | 0.0203 | NA | NA | NA | NA | NA |
| WCM90_Z25_1 | 22 | 17063431 | 51219138 | −0.0854 | NA | NA | NA | NA | NA |
| WCM90_Z25_1 | 3 | 239347 | 197894720 | 0.017 | NA | NA | NA | NA | NA |
| WCM90_Z25_1 | 4 | 59388 | 190426471 | 0.0026 | NA | NA | NA | NA | NA |
| WCM90_Z25_1 | 5 | 140627 | 83402577 | 0.0115 | NA | NA | NA | NA | NA |
| WCM90_Z25_1 | 5 | 83433096 | 121786790 | −0.2443 | NA | NA | NA | NA | NA |
| WCM90_Z25_1 | 5 | 121787002 | 141024595 | 0.0318 | NA | NA | NA | NA | NA |
| WCM90_Z25_1 | 5 | 141024710 | 153857077 | 0.2211 | NA | NA | NA | NA | NA |
| WCM90_Z25_1 | 5 | 153857407 | 170834720 | 0.0606 | NA | NA | NA | NA | NA |
| WCM90_Z25_1 | 5 | 170837557 | 179079332 | 0.2535 | NA | NA | NA | NA | NA |
| WCM90_Z25_1 | 5 | 179105616 | 180687766 | −0.0144 | NA | NA | NA | NA | NA |
| WCM90_Z25_1 | 6 | 203509 | 170893450 | −0.0972 | NA | NA | NA | NA | NA |
| WCM90_Z25_1 | 7 | 193253 | 20449362 | 0.2074 | NA | NA | NA | NA | NA |
| WCM90_Z25_1 | 7 | 20449607 | 22179676 | −0.199 | NA | NA | NA | NA | NA |
| WCM90_Z25_1 | 7 | 22184676 | 26893772 | 0.2315 | NA | NA | NA | NA | NA |
| WCM90_Z25_1 | 7 | 26903991 | 39046460 | −0.0224 | NA | NA | NA | NA | NA |
| WCM90_Z25_1 | 7 | 39125461 | 103828761 | 0.2196 | NA | NA | NA | NA | NA |
| WCM90_Z25_1 | 7 | 103835685 | 124533800 | −0.0303 | NA | NA | NA | NA | NA |
| WCM90_Z25_1 | 7 | 126079219 | 129663496 | 0.2531 | NA | NA | NA | NA | NA |
| WCM90_Z25_1 | 7 | 129664312 | 137588758 | −0.001 | NA | NA | NA | NA | NA |
| WCM90_Z25_1 | 7 | 137590592 | 158937400 | 0.2236 | NA | NA | NA | NA | NA |
| WCM90_Z25_1 | 8 | 190900 | 50449806 | 0.0852 | NA | NA | NA | NA | NA |
| WCM90_Z25_1 | 8 | 51306825 | 66439640 | −0.2181 | NA | NA | NA | NA | NA |
| WCM90_Z25_1 | 8 | 66516719 | 146279508 | 0.0805 | NA | NA | NA | NA | NA |
| WCM90_Z25_1 | 9 | 17086 | 745226 | −0.3743 | NA | NA | NA | NA | NA |
| WCM90_Z25_1 | 9 | 842048 | 38543273 | −0.1386 | NA | NA | NA | NA | NA |
| WCM90_Z25_1 | 9 | 38572051 | 79118447 | −0.3657 | NA | NA | NA | NA | NA |
| WCM90_Z25_1 | 9 | 79118585 | 111617184 | −0.1266 | NA | NA | NA | NA | NA |
| WCM90_Z25_1 | 9 | 111617341 | 121929811 | −0.3428 | NA | NA | NA | NA | NA |
| WCM90_Z25_1 | 9 | 121930197 | 134473918 | −0.0793 | NA | NA | NA | NA | NA |
| WCM90_Z25_1 | 9 | 134477459 | 141111414 | −0.3054 | NA | NA | NA | NA | NA |
| WCM90_Z25_1 | X | 2707688 | 154509348 | −0.0107 | NA | NA | NA | NA | NA |
| WCM90_Z29_1 | 1 | 69603 | 154687414 | −0.1016 | 0.613 | 2.0458 | 1.0131 | 2 | 1 |
| WCM90_Z29_1 | 1 | 154698431 | 155152376 | 0.1867 | 1.34 | NA | NA | NA | NA |
| WCM90_Z29_1 | 1 | 155154668 | 155629629 | 0.4475 | 1.859 | NA | NA | NA | NA |
| WCM90_Z29_1 | 1 | 155629743 | 157014113 | 0.0606 | 1.0485 | 2.2609 | 1.8757 | 2 | 2 |
| WCM90_Z29_1 | 1 | 157062685 | 198700804 | −0.2817 | −0.0031 | 2.0173 | −0.0216 | 2 | 0 |
| WCM90_Z29_1 | 1 | 198701643 | 249212388 | 0.0547 | 1.0342 | 2.0479 | 2.0479 | 2 | 2 |
| WCM90_Z29_1 | 10 | 225952 | 135440086 | −0.1201 | 0.5577 | 3.0594 | −0.1156 | 3 | 0 |
| WCM90_Z29_1 | 11 | 193097 | 2016846 | −0.1464 | 0.4762 | 2.423 | 0.3592 | 2 | 0 |
| WCM90_Z29_1 | 11 | 2017122 | 49229962 | −0.2359 | 0.1722 | 1.1722 | 1.0814 | 1 | 1 |
| WCM90_Z29_1 | 11 | 49598081 | 61404455 | −0.1289 | 0.5306 | 2.1245 | 0.7645 | 2 | 1 |
| WCM90_Z29_1 | 11 | 61404866 | 82880683 | 0.0819 | 1.1002 | 2.1593 | 2.1284 | 2 | 2 |
| WCM90_Z29_1 | 11 | 82880850 | 82938815 | −0.2558 | 0.0981 | NA | NA | NA | NA |
| WCM90_Z29_1 | 11 | 82947489 | 96124759 | −0.0955 | 0.6311 | 2.2265 | 0.871 | 2 | 1 |
| WCM90_Z29_1 | 11 | 96124891 | 134856563 | −0.2776 | 0.0134 | 2.0959 | −0.0772 | 2 | 0 |
| WCM90_Z29_1 | 12 | 148644 | 121666646 | 0.0616 | 1.0509 | 2.1226 | 2.0212 | 2 | 2 |
| WCM90_Z29_1 | 12 | 121666868 | 133254274 | −0.2595 | 0.084 | 2.1236 | −0.0038 | 2 | 0 |
| WCM90_Z29_1 | 12 | 133256221 | 133810703 | 0.1253 | 1.2022 | 2.656 | 1.9457 | 3 | 2 |
| WCM90_Z29_1 | 13 | 19600475 | 115089411 | −0.2244 | 0.2141 | 2.3081 | 0.0118 | 2 | 0 |
| WCM90_Z29_1 | 14 | 19377710 | 107283210 | 0.0474 | 1.0161 | 2.0727 | 1.9723 | 2 | 2 |
| WCM90_Z29_1 | 15 | 20170150 | 52258248 | −0.2943 | −0.0542 | 1.0344 | 0.8918 | 1 | 1 |
| WCM90_Z29_1 | 15 | 52258698 | 102517662 | −0.1527 | 0.4562 | 1.8135 | 0.9304 | 2 | 1 |
| WCM90_Z29_1 | 16 | 67604 | 114934 | 0.148 | 1.2539 | NA | NA | NA | NA |
| WCM90_Z29_1 | 16 | 115009 | 25258035 | 0.2085 | 1.3872 | 2.6831 | 2.5483 | 3 | 3 |
| WCM90_Z29_1 | 16 | 25258450 | 90233622 | 0.1251 | 1.2017 | 3.0421 | 1.5581 | 3 | 2 |
| WCM90_Z29_1 | 17 | 131595 | 21201719 | −0.2694 | 0.0458 | 2.1129 | −0.0484 | 2 | 0 |
| WCM90_Z29_1 | 17 | 21202191 | 36357236 | 0.0469 | 1.0147 | 2.1861 | 1.8547 | 2 | 2 |
| WCM90_Z29_1 | 17 | 36358127 | 37785806 | −0.2821 | −0.0045 | NA | NA | NA | NA |
| WCM90_Z29_1 | 17 | 37786257 | 81083419 | 0.0655 | 1.0605 | 2.1314 | 2.0399 | 2 | 2 |
| WCM90_Z29_1 | 18 | 48216 | 78005226 | −0.0271 | 0.8233 | 1.8553 | 1.6836 | 2 | 2 |
| WCM90_Z29_1 | 19 | 281499 | 1990074 | −0.232 | 0.1865 | 1.2414 | 1.0345 | 1 | 1 |
| WCM90_Z29_1 | 19 | 1990752 | 4171129 | 0.0555 | 1.036 | 3.192 | 0.9091 | 3 | 1 |
| WCM90_Z29_1 | 19 | 4171445 | 9325126 | −0.2677 | 0.0521 | 1.1054 | 0.9682 | 1 | 1 |
| WCM90_Z29_1 | 19 | 9325252 | 17015340 | −0.0727 | 0.6969 | 2.134 | 1.1081 | 2 | 1 |
| WCM90_Z29_1 | 19 | 17017878 | 21560270 | 0.0755 | 1.0847 | 2.2592 | 1.9827 | 2 | 2 |
| WCM90_Z29_1 | 19 | 21580051 | 24309712 | −0.062 | 0.7273 | 2.554 | 0.757 | 3 | 1 |
| WCM90_Z29_1 | 19 | 24310032 | 38943503 | 0.449 | 1.8617 | 5.3593 | 1.9095 | 5 | 2 |
| WCM90_Z29_1 | 19 | 38945906 | 49631692 | −0.2812 | −8.00E−04 | 1.9913 | 0.0076 | 2 | 0 |
| WCM90_Z29_1 | 19 | 49632212 | 57157157 | 0.5367 | 2.018 | 6.1089 | 1.9916 | 6 | 2 |
| WCM90_Z29_1 | 19 | 57158960 | 59083988 | 0.0673 | 1.0648 | 2.1989 | 1.985 | 2 | 2 |
| WCM90_Z29_1 | 2 | 41609 | 60687690 | 0.0591 | 1.0449 | 2.0632 | 2.0632 | 2 | 2 |
| WCM90_Z29_1 | 2 | 60687959 | 61715389 | −0.1999 | 0.3 | NA | NA | NA | NA |
| WCM90_Z29_1 | 2 | 61715779 | 116593770 | 0.0538 | 1.032 | 2.1154 | 1.9742 | 2 | 2 |
| WCM90_Z29_1 | 2 | 116594117 | 166535708 | −0.2574 | 0.0921 | 2.1759 | −0.0441 | 2 | 0 |
| WCM90_Z29_1 | 2 | 166535918 | 242946607 | 0.0474 | 1.016 | 2.0524 | 1.9922 | 2 | 2 |
| WCM90_Z29_1 | 20 | 68397 | 62904890 | 0.5009 | 1.9551 | 3.8773 | 3.8773 | 4 | 4 |
| WCM90_Z29_1 | 21 | 9907291 | 48084277 | −0.1123 | 0.5812 | 2.0176 | 0.9745 | 2 | 1 |
| WCM90_Z29_1 | 22 | 17063485 | 51219138 | −0.0803 | 0.6754 | 2.0889 | 1.1051 | 2 | 1 |
| WCM90_Z29_1 | 3 | 239347 | 197894712 | 0.0471 | 1.0152 | 2.0512 | 1.991 | 2 | 2 |
| WCM90_Z29_1 | 4 | 59390 | 126411214 | 0.033 | 0.98 | 2.0072 | 1.9376 | 2 | 2 |
| WCM90_Z29_1 | 4 | 126411401 | 130032843 | −0.2902 | −0.0375 | NA | NA | NA | NA |
| WCM90_Z29_1 | 4 | 133357020 | 190426459 | 0.0436 | 1.0067 | 2.0794 | 1.9391 | 2 | 2 |
| WCM90_Z29_1 | 5 | 140443 | 31294351 | −0.1401 | 0.496 | 1.4853 | 1.3353 | 1 | 1 |
| WCM90_Z29_1 | 5 | 31297501 | 40681254 | 0.6393 | 2.1922 | 7.1209 | 2.0188 | 7 | 2 |
| WCM90_Z29_1 | 5 | 40681530 | 83433096 | 0.0054 | 0.9095 | 1.976 | 1.7808 | 2 | 2 |
| WCM90_Z29_1 | 5 | 83476228 | 122139220 | −0.2905 | −0.0386 | 1.8716 | 0.0756 | 2 | 0 |
| WCM90_Z29_1 | 5 | 122143973 | 154065352 | 0.096 | 1.1338 | 2.2724 | 2.1165 | 2 | 2 |
| WCM90_Z29_1 | 5 | 154092631 | 170380610 | −0.0967 | 0.6276 | 1.8041 | 1.2859 | 2 | 1 |
| WCM90_Z29_1 | 5 | 170395368 | 179079358 | 0.1961 | 1.3604 | 2.8014 | 2.3337 | 3 | 2 |
| WCM90_Z29_1 | 5 | 179105614 | 180687776 | −0.133 | 0.518 | 2.0924 | 0.7716 | 2 | 1 |
| WCM90_Z29_1 | 6 | 203615 | 57046862 | −0.0966 | 0.6279 | 3.1225 | −0.0319 | 3 | 0 |
| WCM90_Z29_1 | 6 | 57048783 | 106536261 | −0.266 | 0.0588 | 1.9752 | 0.108 | 2 | 0 |
| WCM90_Z29_1 | 6 | 106543515 | 170893454 | −0.1269 | 0.5367 | 3.0218 | −0.1204 | 3 | 0 |
| WCM90_Z29_1 | 7 | 193222 | 14620521 | 0.4437 | 1.852 | 6.2739 | 0.9462 | 6 | 1 |
| WCM90_Z29_1 | 7 | 14622748 | 20449617 | 0.2317 | 1.4366 | NA | NA | NA | NA |
| WCM90_Z29_1 | 7 | 20662961 | 21985409 | −0.4412 | −0.787 | NA | NA | NA | NA |
| WCM90_Z29_1 | 7 | 22162090 | 117876935 | 0.1663 | 1.2949 | 3.9262 | 0.9812 | 4 | 1 |
| WCM90_Z29_1 | 7 | 117880031 | 158937375 | −0.1186 | 0.5622 | 1.9331 | 1.0199 | 2 | 1 |
| WCM90_Z29_1 | 8 | 190908 | 33454982 | −0.4785 | −1.035 | 1.0008 | −0.0247 | 1 | 0 |
| WCM90_Z29_1 | 8 | 35406923 | 43172516 | 0.4657 | 1.8921 | 6.0072 | 1.4164 | 6 | 1 |
| WCM90_Z29_1 | 8 | 43173764 | 49987801 | −0.062 | 0.7272 | NA | NA | NA | NA |
| WCM90_Z29_1 | 8 | 50449807 | 66439647 | −0.2834 | −0.0097 | 1.8798 | 0.1068 | 2 | 0 |
| WCM90_Z29_1 | 8 | 66516809 | 146279529 | −0.0978 | 0.6242 | 1.9909 | 1.0918 | 2 | 1 |
| WCM90_Z29_1 | 9 | 17116 | 39078450 | −0.1211 | 0.5547 | 2.1347 | 0.803 | 2 | 1 |
| WCM90_Z29_1 | 9 | 39086729 | 141111414 | −0.2656 | 0.0603 | 1.0872 | 0.9982 | 1 | 1 |
| WCM90_Z29_1 | X | 2707694 | 5811191 | −0.0546 | 0.748 | NA | NA | NA | NA |
| WCM90_Z29_1 | X | 5811678 | 23892572 | −0.2988 | −0.0727 | NA | NA | NA | NA |
| WCM90_Z29_1 | X | 23897107 | 29417278 | 0.251 | 1.4768 | NA | NA | NA | NA |
| WCM90_Z29_1 | X | 29592467 | 33229421 | 0.9459 | 2.6695 | NA | NA | NA | NA |
| WCM90_Z29_1 | X | 34148186 | 64196239 | 0.0178 | 0.9415 | NA | NA | NA | NA |
| WCM90_Z29_1 | X | 64708913 | 69397514 | 1.7398 | 3.7259 | NA | NA | NA | NA |
| WCM90_Z29_1 | X | 69419145 | 154509348 | −0.0033 | 0.8867 | NA | NA | NA | NA |
| WCM91 | 1 | 15922 | 249213973 | −0.0095 | NA | NA | NA | NA | NA |
| WCM91 | 10 | 225958 | 135438943 | −0.025 | NA | NA | NA | NA | NA |
| WCM91 | 11 | 193096 | 134605959 | −0.0225 | NA | NA | NA | NA | NA |
| WCM91 | 12 | 148468 | 133810788 | −0.0415 | NA | NA | NA | NA | NA |
| WCM91 | 13 | 19041937 | 115089411 | −0.0409 | NA | NA | NA | NA | NA |
| WCM91 | 14 | 19110239 | 107283213 | −0.0406 | NA | NA | NA | NA | NA |
| WCM91 | 15 | 20083921 | 102506257 | 0.0053 | NA | NA | NA | NA | NA |
| WCM91 | 16 | 67619 | 90260461 | −0.0319 | NA | NA | NA | NA | NA |
| WCM91 | 17 | 263028 | 81052545 | −0.0344 | NA | NA | NA | NA | NA |
| WCM91 | 18 | 47732 | 78005203 | −0.0585 | NA | NA | NA | NA | NA |
| WCM91 | 19 | 105021 | 59093696 | −0.0166 | NA | NA | NA | NA | NA |
| WCM91 | 2 | 41612 | 243037103 | −0.0208 | NA | NA | NA | NA | NA |
| WCM91 | 20 | 68342 | 62934592 | −0.0214 | NA | NA | NA | NA | NA |
| WCM91 | 21 | 9590271 | 48084175 | −0.022 | NA | NA | NA | NA | NA |
| WCM91 | 22 | 16159060 | 51219129 | −0.0221 | NA | NA | NA | NA | NA |
| WCM91 | 3 | 361470 | 197896800 | −0.0239 | NA | NA | NA | NA | NA |
| WCM91 | 4 | 53403 | 190873327 | −0.0231 | NA | NA | NA | NA | NA |
| WCM91 | 5 | 90696 | 180687610 | −0.0343 | NA | NA | NA | NA | NA |
| WCM91 | 6 | 203535 | 170892854 | −0.0356 | NA | NA | NA | NA | NA |
| WCM91 | 7 | 193222 | 158937104 | −0.0247 | NA | NA | NA | NA | NA |
| WCM91 | 8 | 166443 | 146279528 | −0.0398 | NA | NA | NA | NA | NA |
| WCM91 | 9 | 116931 | 141071668 | −0.0092 | NA | NA | NA | NA | NA |
| WCM91 | X | 2700162 | 154493562 | −0.2438 | NA | NA | NA | NA | NA |
| WCM91_Z1_1 | 1 | 69603 | 9780597 | 0.0604 | 0.8091 | 2.4946 | 1.0097 | 2 | 1 |
| WCM91_Z1_1 | 1 | 9780843 | 111964236 | −0.1309 | 0.5784 | 1.9863 | 1 | 2 | 1 |
| WCM91_Z1_1 | 1 | 111965553 | 112991698 | −0.5314 | 0.0709 | NA | NA | NA | NA |
| WCM91_Z1_1 | 1 | 112997110 | 116932317 | −0.1402 | 0.567 | 1.9623 | 1.0005 | 2 | 1 |
| WCM91_Z1_1 | 1 | 116932921 | 116952766 | −0.0259 | 0.7058 | NA | NA | NA | NA |
| WCM91_Z1_1 | 1 | 117037377 | 117603156 | 0.7842 | 1.6374 | NA | NA | NA | NA |
| WCM91_Z1_1 | 1 | 117605058 | 117622238 | 1.6651 | 2.5873 | NA | NA | NA | NA |
| WCM91_Z1_1 | 1 | 117624586 | 118574329 | 0.9974 | 1.8716 | NA | NA | NA | NA |
| WCM91_Z1_1 | 1 | 118579398 | 118727707 | 0.8975 | 1.7623 | NA | NA | NA | NA |
| WCM91_Z1_1 | 1 | 119427406 | 145096591 | −0.1059 | 0.6089 | NA | NA | NA | NA |
| WCM91_Z1_1 | 1 | 145103947 | 151079544 | 0.8043 | 1.6595 | 4.5597 | 1.7587 | 5 | 2 |
| WCM91_Z1_1 | 1 | 151089888 | 152331302 | 1.1707 | 2.0597 | 6.3392 | 1.9989 | 6 | 2 |
| WCM91_Z1_1 | 1 | 152382077 | 152957970 | −0.4309 | 0.2019 | NA | NA | NA | NA |
| WCM91_Z1_1 | 1 | 152975619 | 154243372 | 0.3234 | 1.1172 | 2.3219 | 2.0166 | 2 | 2 |
| WCM91_Z1_1 | 1 | 154246028 | 155162235 | 1.1087 | 1.9927 | 6.0457 | 1.9138 | 6 | 2 |
| WCM91_Z1_1 | 1 | 155162615 | 170959088 | −0.402 | 0.2391 | 1.9855 | 0.375 | 2 | 0 |
| WCM91_Z1_1 | 1 | 170961433 | 174210796 | 0.1892 | 0.9612 | 1.9925 | 1.9013 | 2 | 2 |
| WCM91_Z1_1 | 1 | 174219725 | 178442258 | −0.4483 | 0.1794 | 2.1526 | 0.1123 | 2 | 0 |
| WCM91_Z1_1 | 1 | 178482329 | 205689741 | 0.2137 | 0.9899 | 1.986 | 1.986 | 2 | 2 |
| WCM91_Z1_1 | 1 | 205696890 | 207200892 | −0.5563 | 0.0381 | 2.0876 | −0.0341 | 2 | 0 |
| WCM91_Z1_1 | 1 | 207222546 | 207653394 | 0.1785 | 0.9487 | NA | NA | NA | NA |
| WCM91_Z1_1 | 1 | 207669702 | 207975820 | −0.5982 | −0.0177 | NA | NA | NA | NA |
| WCM91_Z1_1 | 1 | 207990748 | 213445936 | 0.214 | 0.9902 | 2.0189 | 1.954 | 2 | 2 |
| WCM91_Z1_1 | 1 | 214170086 | 249212558 | 0.6289 | 1.4643 | 3.4954 | 2.0233 | 3 | 2 |
| WCM91_Z1_1 | 10 | 226016 | 15902237 | −0.5865 | −0.002 | 1.0199 | 0.9773 | 1 | 1 |
| WCM91_Z1_1 | 10 | 16526397 | 17243618 | −1.2683 | −1.0088 | 0.9661 | 0.0278 | 1 | 0 |
| WCM91_Z1_1 | 10 | 17271587 | 50386260 | −0.1087 | 0.6055 | 2.0204 | 1.0225 | 2 | 1 |
| WCM91_Z1_1 | 10 | 50396343 | 50740980 | −0.6589 | −0.0994 | NA | NA | NA | NA |
| WCM91_Z1_1 | 10 | 50818812 | 60966553 | 0.2302 | 1.0091 | 2.0972 | 1.9283 | 2 | 2 |
| WCM91_Z1_1 | 10 | 60994064 | 61122227 | −0.6964 | −0.1505 | NA | NA | NA | NA |
| WCM91_Z1_1 | 10 | 61412538 | 88681437 | 0.2203 | 0.9975 | 2.0082 | 1.9849 | 2 | 2 |
| WCM91_Z1_1 | 10 | 88683169 | 90122360 | −0.6158 | −0.0412 | NA | NA | NA | NA |
| WCM91_Z1_1 | 10 | 90332716 | 93558531 | 0.2473 | 1.0291 | 2.0407 | 2.0407 | 2 | 2 |
| WCM91_Z1_1 | 10 | 93572874 | 95072891 | −0.5851 | −2.00E−04 | NA | NA | NA | NA |
| WCM91_Z1_1 | 10 | 95076498 | 135440081 | −0.1244 | 0.5864 | 2.0152 | 0.9877 | 2 | 1 |
| WCM91_Z1_1 | 11 | 193146 | 62886468 | −0.1295 | 0.5801 | 2.0033 | 0.9867 | 2 | 1 |
| WCM91_Z1_1 | 11 | 62886727 | 85367358 | 0.2085 | 0.9838 | 1.9777 | 1.9777 | 2 | 2 |
| WCM91_Z1_1 | 11 | 85373391 | 134856597 | 0.205 | 0.9797 | 2.998 | 0.9462 | 3 | 1 |
| WCM91_Z1_1 | 12 | 148674 | 20896242 | −0.2701 | 0.4062 | 2.6653 | −0.0149 | 3 | 0 |
| WCM91_Z1_1 | 12 | 20903659 | 26231979 | 0.7914 | 1.6454 | 3.329 | 2.9275 | 3 | 3 |
| WCM91_Z1_1 | 12 | 26274965 | 76796498 | −0.276 | 0.3988 | 2.6201 | 0.0168 | 3 | 0 |
| WCM91_Z1_1 | 12 | 76804371 | 121758193 | 0.4026 | 1.2082 | 2.3475 | 2.2735 | 2 | 2 |
| WCM91_Z1_1 | 12 | 122064924 | 122962804 | −0.0276 | 0.7038 | 2.3018 | 0.9558 | 2 | 1 |
| WCM91_Z1_1 | 12 | 122964798 | 133810751 | 0.4006 | 1.2059 | 2.5734 | 2.0401 | 3 | 2 |
| WCM91_Z1_1 | 13 | 19600515 | 70681598 | −0.5663 | 0.0248 | 2.0587 | −0.024 | 2 | 0 |
| WCM91_Z1_1 | 13 | 72014808 | 72255975 | −2.4292 | −4.7173 | NA | NA | NA | NA |
| WCM91_Z1_1 | 13 | 72440256 | 94938655 | −0.5947 | −0.013 | 1.9975 | −0.0155 | 2 | 0 |
| WCM91_Z1_1 | 13 | 94958373 | 95735530 | −2.3566 | −4.1283 | NA | NA | NA | NA |
| WCM91_Z1_1 | 13 | 95768240 | 115089411 | −0.5753 | 0.0129 | 2.0264 | −0.0085 | 2 | 0 |
| WCM91_Z1_1 | 14 | 19377703 | 107283210 | −0.2907 | 0.3803 | 1.6319 | 0.9714 | 2 | 1 |
| WCM91_Z1_1 | 15 | 20170150 | 83358196 | −0.1374 | 0.5704 | 1.974 | 0.9958 | 2 | 1 |
| WCM91_Z1_1 | 15 | 83378388 | 83561552 | −1.1962 | −0.8902 | NA | NA | NA | NA |
| WCM91_Z1_1 | 15 | 83657568 | 102506284 | −0.1308 | 0.5785 | 1.9646 | 1.0221 | 2 | 1 |
| WCM91_Z1_1 | 16 | 67604 | 90233299 | 0.0994 | 0.8554 | 2.6321 | 0.9865 | 3 | 1 |
| WCM91_Z1_1 | 17 | 131595 | 6733672 | 0.1908 | 0.963 | 1.9494 | 1.9494 | 2 | 2 |
| WCM91_Z1_1 | 17 | 6888795 | 8633488 | −0.5848 | 2.00E−04 | 2.0229 | −0.0226 | 2 | 0 |
| WCM91_Z1_1 | 17 | 8638480 | 10447278 | 0.2272 | 1.0056 | 2.0335 | 1.982 | 2 | 2 |
| WCM91_Z1_1 | 17 | 10448738 | 19748662 | −0.5948 | −0.0131 | 1.9802 | 0.0017 | 2 | 0 |
| WCM91_Z1_1 | 17 | 19752713 | 43176872 | 0.2179 | 0.9948 | 2.0114 | 1.9742 | 2 | 2 |
| WCM91_Z1_1 | 17 | 43180331 | 44632740 | −0.7355 | −0.2045 | NA | NA | NA | NA |
| WCM91_Z1_1 | 17 | 44770430 | 65212042 | 0.2098 | 0.9853 | 2.0075 | 1.952 | 2 | 2 |
| WCM91_Z1_1 | 17 | 65214766 | 81083419 | −0.1301 | 0.5794 | 1.9767 | 1.0117 | 2 | 1 |
| WCM91_Z1_1 | 18 | 48200 | 78005224 | −0.5714 | 0.0181 | 2.0409 | −0.0157 | 2 | 0 |
| WCM91_Z1_1 | 19 | 281424 | 8455333 | −0.5975 | −0.0167 | 1.9997 | −0.0227 | 2 | 0 |
| WCM91_Z1_1 | 19 | 8464856 | 59083970 | 0.2012 | 0.9752 | 1.9774 | 1.9544 | 2 | 2 |
| WCM91_Z1_1 | 2 | 41612 | 56603065 | −0.1228 | 0.5883 | 1.9865 | 1.0204 | 2 | 1 |
| WCM91_Z1_1 | 2 | 56611401 | 58468432 | −1.264 | −1.0017 | NA | NA | NA | NA |
| WCM91_Z1_1 | 2 | 60614577 | 240500560 | −0.1229 | 0.5882 | 1.9974 | 1.0093 | 2 | 1 |
| WCM91_Z1_1 | 2 | 240504712 | 242946607 | −0.4034 | 0.2373 | 1.9984 | 0.3592 | 2 | 0 |
| WCM91_Z1_1 | 20 | 68375 | 2517926 | 0.1827 | 0.9536 | 2.0207 | 1.8527 | 2 | 2 |
| WCM91_Z1_1 | 20 | 2539274 | 3585650 | −0.6148 | −0.0399 | 1.951 | −0.0055 | 2 | 0 |
| WCM91_Z1_1 | 20 | 3605206 | 62904925 | 0.2062 | 0.9811 | 1.9739 | 1.9739 | 2 | 2 |
| WCM91_Z1_1 | 21 | 9826207 | 48084253 | −0.1426 | 0.5641 | 2.0039 | 0.953 | 2 | 1 |
| WCM91_Z1_1 | 22 | 16287443 | 51219138 | −0.1652 | 0.5364 | 1.9498 | 0.9509 | 2 | 1 |
| WCM91_Z1_1 | 3 | 239398 | 71161716 | 0.2196 | 0.9967 | 1.9954 | 1.9954 | 2 | 2 |
| WCM91_Z1_1 | 3 | 71179670 | 73432800 | −0.6262 | −0.0552 | NA | NA | NA | NA |
| WCM91_Z1_1 | 3 | 73433185 | 197896720 | 0.217 | 0.9937 | 1.9983 | 1.9843 | 2 | 2 |
| WCM91_Z1_1 | 4 | 59326 | 25235857 | −0.1373 | 0.5706 | 1.9652 | 1.005 | 2 | 1 |
| WCM91_Z1_1 | 4 | 25254012 | 57976479 | −1.211 | −0.9143 | 1.0636 | −0.0024 | 1 | 0 |
| WCM91_Z1_1 | 4 | 61788378 | 159165453 | −0.1328 | 0.576 | 2.0106 | 0.9709 | 2 | 1 |
| WCM91_Z1_1 | 4 | 159174637 | 190426570 | −1.2 | −0.8964 | 1.0709 | 0.0036 | 1 | 0 |
| WCM91_Z1_1 | 5 | 140666 | 53467650 | 0.2119 | 0.9877 | 2.0016 | 1.9645 | 2 | 2 |
| WCM91_Z1_1 | 5 | 53606277 | 54737696 | −0.6402 | −0.0741 | NA | NA | NA | NA |
| WCM91_Z1_1 | 5 | 54763765 | 58021848 | −2.4768 | −5.2683 | NA | NA | NA | NA |
| WCM91_Z1_1 | 5 | 58120955 | 150257586 | −0.5666 | 0.0245 | 2.0502 | −0.016 | 2 | 0 |
| WCM91_Z1_1 | 5 | 150275227 | 150566946 | 0.6439 | 1.4811 | NA | NA | NA | NA |
| WCM91_Z1_1 | 5 | 150578592 | 180687776 | 0.1919 | 0.9644 | 1.9604 | 1.9421 | 2 | 2 |
| WCM91_Z1_1 | 6 | 203515 | 109563427 | −0.1509 | 0.5539 | 1.9419 | 0.9943 | 2 | 1 |
| WCM91_Z1_1 | 6 | 109575770 | 170892772 | 0.2016 | 0.9757 | 1.9666 | 1.9666 | 2 | 2 |
| WCM91_Z1_1 | 7 | 193205 | 14758296 | 0.224 | 1.0019 | 2.026 | 1.9793 | 2 | 2 |
| WCM91_Z1_1 | 7 | 14775720 | 22170598 | −0.5154 | 0.092 | 2.1111 | 0.0207 | 2 | 0 |
| WCM91_Z1_1 | 7 | 22175743 | 96747098 | 0.2079 | 0.9831 | 1.9929 | 1.9605 | 2 | 2 |
| WCM91_Z1_1 | 7 | 96810356 | 105148851 | −0.1228 | 0.5883 | 1.9664 | 1.0406 | 2 | 1 |
| WCM91_Z1_1 | 7 | 105172804 | 111634284 | 0.2064 | 0.9813 | NA | NA | NA | NA |
| WCM91_Z1_1 | 7 | 111638506 | 111982645 | −0.6741 | −0.1202 | NA | NA | NA | NA |
| WCM91_Z1_1 | 7 | 112090747 | 158937011 | 0.2102 | 0.9858 | 1.9804 | 1.9804 | 2 | 2 |
| WCM91_Z1_1 | 8 | 190900 | 37689026 | −0.5831 | 0.0025 | 2.0164 | −0.0129 | 2 | 0 |
| WCM91_Z1_1 | 8 | 37690607 | 146279416 | 0.6365 | 1.4729 | 3.5841 | 1.9674 | 4 | 2 |
| WCM91_Z1_1 | 9 | 116832 | 131115438 | 0.0011 | 0.7383 | 1.9605 | 1.376 | 2 | 1 |
| WCM91_Z1_1 | 9 | 131115784 | 136550376 | −0.9933 | −0.5749 | 1.3575 | −0.0149 | 1 | 0 |
| WCM91_Z1_1 | 9 | 136555629 | 141111414 | 0.0181 | 0.7587 | 2.0038 | 1.38 | 2 | 1 |
| WCM91_Z1_1 | X | 2700147 | 20081599 | 0.9618 | 1.8328 | NA | NA | NA | NA |
| WCM91_Z1_1 | X | 20134945 | 57936542 | 0.7402 | 1.5885 | NA | NA | NA | NA |
| WCM91_Z1_1 | X | 62898360 | 65486458 | 1.4266 | 2.3343 | NA | NA | NA | NA |
| WCM91_Z1_1 | X | 65819524 | 66943685 | 3.3069 | 4.2824 | NA | NA | NA | NA |
| WCM91_Z1_1 | X | 67268291 | 154509348 | −0.6037 | −0.0251 | NA | NA | NA | NA |
| WCM9_X1_1 | 1 | 721450 | 59042563 | −0.0131 | 0.8658 | 2.6238 | 1.021 | 3 | 1 |
| WCM9_X1_1 | 1 | 59042734 | 79357292 | −0.4094 | −0.785 | 1.2246 | −0.0639 | 1 | 0 |
| WCM9_X1_1 | 1 | 79358827 | 249212366 | −0.0101 | 0.8741 | 2.5271 | 1.1387 | 3 | 1 |
| WCM9_X1_1 | 10 | 243934 | 135440117 | −0.0196 | 0.8473 | 2.4425 | 1.1557 | 2 | 1 |
| WCM9_X1_1 | 11 | 193127 | 123886681 | −0.0051 | 0.8882 | 2.5423 | 1.1595 | 3 | 1 |
| WCM9_X1_1 | 11 | 123887066 | 134856592 | −0.1544 | 0.4222 | 2.5073 | 0.1727 | 3 | 0 |
| WCM9_X1_1 | 12 | 148642 | 111072571 | 0.1822 | 1.3629 | 2.5897 | 2.5542 | 3 | 3 |
| WCM9_X1_1 | 12 | 111074280 | 115120975 | −0.2067 | 0.2301 | 1.9867 | 0.3592 | 2 | 0 |
| WCM9_X1_1 | 12 | 115801948 | 133779951 | 0.0043 | 0.9146 | 2.2723 | 1.4978 | 2 | 1 |
| WCM9_X1_1 | 12 | 133780309 | 133810703 | −0.0539 | 0.7469 | NA | NA | NA | NA |
| WCM9_X1_1 | 13 | 19600147 | 48833056 | 0.2849 | 1.5891 | 3.2185 | 2.7989 | 3 | 3 |
| WCM9_X1_1 | 13 | 48835315 | 115089411 | −0.2093 | 0.22 | 2.3696 | −0.0401 | 2 | 0 |
| WCM9_X1_1 | 14 | 19377701 | 21840847 | 0.098 | 1.1611 | 2.7165 | 1.756 | 3 | 2 |
| WCM9_X1_1 | 14 | 21851781 | 78398036 | 0.1532 | 1.2952 | 2.4541 | 2.4541 | 2 | 2 |
| WCM9_X1_1 | 14 | 78399592 | 107283019 | 7.00E−04 | 0.9046 | 2.4201 | 1.3239 | 2 | 1 |
| WCM9_X1_1 | 15 | 20170019 | 102514159 | −0.0187 | 0.8499 | 2.441 | 1.1638 | 2 | 1 |
| WCM9_X1_1 | 16 | 66930 | 18903638 | 0.0131 | 0.9389 | 2.5646 | 1.2695 | 3 | 1 |
| WCM9_X1_1 | 16 | 18908268 | 90086249 | 0.1559 | 1.3016 | 2.5289 | 2.401 | 3 | 2 |
| WCM9_X1_1 | 16 | 90086471 | 90233294 | −0.0086 | 0.8786 | 2.6736 | 1.0036 | 3 | 1 |
| WCM9_X1_1 | 17 | 131595 | 10555851 | −0.2049 | 0.2371 | 2.4551 | −0.0978 | 2 | 0 |
| WCM9_X1_1 | 17 | 10558376 | 30981567 | −0.0014 | 0.8987 | 2.5628 | 1.166 | 3 | 1 |
| WCM9_X1_1 | 17 | 30986163 | 34948538 | −0.2208 | 0.1749 | 2.5212 | −0.2634 | 3 | 0 |
| WCM9_X1_1 | 17 | 34951424 | 36552222 | 0.1723 | 1.3399 | NA | NA | NA | NA |
| WCM9_X1_1 | 17 | 36704045 | 38991032 | −0.2308 | 0.1349 | 2.823 | −0.627 | 3 | −1 |
| WCM9_X1_1 | 17 | 38991476 | 39634674 | 0.0283 | 0.9802 | NA | NA | NA | NA |
| WCM9_X1_1 | 17 | 39635231 | 46265278 | 0.2475 | 1.5089 | 2.9265 | 2.7655 | 3 | 3 |
| WCM9_X1_1 | 17 | 46266848 | 49365936 | 0.042 | 1.017 | 2.2386 | 1.8089 | 2 | 2 |
| WCM9_X1_1 | 17 | 49371340 | 58711271 | −0.1965 | 0.2692 | 2.571 | −0.1608 | 3 | 0 |
| WCM9_X1_1 | 17 | 58725437 | 60837229 | 0.1193 | 1.2135 | NA | NA | NA | NA |
| WCM9_X1_1 | 17 | 60879042 | 64225437 | 0.4313 | 1.8825 | 4.8621 | 2.5124 | 5 | 3 |
| WCM9_X1_1 | 17 | 64299011 | 65103522 | −0.141 | 0.4686 | NA | NA | NA | NA |
| WCM9_X1_1 | 17 | 65103784 | 81083507 | −0.2073 | 0.228 | 2.4411 | −0.0986 | 2 | 0 |
| WCM9_X1_1 | 18 | 48224 | 580797 | −0.1594 | 0.4047 | NA | NA | NA | NA |
| WCM9_X1_1 | 18 | 617996 | 29340380 | −0.0031 | 0.894 | 2.254 | 1.4627 | 2 | 1 |
| WCM9_X1_1 | 18 | 29340529 | 46448256 | −0.3787 | −0.5889 | 1.3269 | 0.0028 | 1 | 0 |
| WCM9_X1_1 | 18 | 46468946 | 67231807 | −0.1966 | 0.2686 | 1.3048 | 1.1045 | 1 | 1 |
| WCM9_X1_1 | 18 | 67266700 | 78005194 | −0.3498 | −0.4228 | 1.0244 | 0.4676 | 1 | 0 |
| WCM9_X1_1 | 19 | 281433 | 59083951 | −0.0199 | 0.8463 | 2.5286 | 1.0672 | 3 | 1 |
| WCM9_X1_1 | 2 | 41615 | 242946616 | −0.0151 | 0.8601 | 2.4038 | 1.2266 | 2 | 1 |
| WCM9_X1_1 | 20 | 68375 | 62904848 | 0.1573 | 1.3047 | 2.5285 | 2.4122 | 3 | 2 |
| WCM9_X1_1 | 21 | 9826207 | 48084210 | −0.2375 | 0.1077 | 1.1439 | 1.0111 | 1 | 1 |
| WCM9_X1_1 | 22 | 17060362 | 40364102 | −0.2024 | 0.2468 | 1.2501 | 1.1231 | 1 | 1 |
| WCM9_X1_1 | 22 | 40365458 | 51159824 | −0.0296 | 0.8184 | 2.4075 | 1.1194 | 2 | 1 |
| WCM9_X1_1 | 22 | 51160242 | 51215157 | −0.2172 | 0.1894 | NA | NA | NA | NA |
| WCM9_X1_1 | 3 | 239405 | 52437893 | −0.147 | 0.448 | 2.74 | −0.0118 | 3 | 0 |
| WCM9_X1_1 | 3 | 52438553 | 54880461 | −0.4438 | −1.0355 | 0.7602 | 0.2155 | 1 | 0 |
| WCM9_X1_1 | 3 | 54905620 | 97499470 | 0.0298 | 0.9843 | 3.6562 | 0.3006 | 4 | 0 |
| WCM9_X1_1 | 3 | 97503887 | 121810467 | 0.1096 | 1.1899 | 3.7505 | 0.8121 | 4 | 1 |
| WCM9_X1_1 | 3 | 121822524 | 141164413 | 0.3408 | 1.7048 | 3.3127 | 3.207 | 3 | 3 |
| WCM9_X1_1 | 3 | 141164729 | 142561804 | 0.1723 | 1.3399 | 4.1363 | 0.9263 | 4 | 1 |
| WCM9_X1_1 | 3 | 142562146 | 164059210 | 0.0121 | 0.9362 | 3.9706 | −0.1437 | 4 | 0 |
| WCM9_X1_1 | 3 | 164697153 | 185826295 | 0.3327 | 1.6883 | 3.4002 | 3.0454 | 3 | 3 |
| WCM9_X1_1 | 3 | 185867924 | 197896714 | −0.0182 | 0.8513 | 3.427 | 0.1813 | 3 | 0 |
| WCM9_X1_1 | 4 | 59331 | 190396288 | −0.2011 | 0.2517 | 2.4477 | −0.0666 | 2 | 0 |
| WCM9_X1_1 | 5 | 140636 | 180687814 | −0.0091 | 0.877 | 2.2824 | 1.3907 | 2 | 1 |
| WCM9_X1_1 | 6 | 203583 | 25554247 | −0.1641 | 0.388 | 2.3516 | 0.2655 | 2 | 0 |
| WCM9_X1_1 | 6 | 25557017 | 84233628 | −0.0067 | 0.8839 | 2.3076 | 1.3832 | 2 | 1 |
| WCM9_X1_1 | 6 | 84233831 | 151674270 | −0.1852 | 0.3114 | 2.4753 | 0.0066 | 2 | 0 |
| WCM9_X1_1 | 6 | 151674506 | 170156857 | −0.0212 | 0.8427 | 2.2972 | 1.2895 | 2 | 1 |
| WCM9_X1_1 | 6 | 170159096 | 170893454 | −0.1009 | 0.6013 | NA | NA | NA | NA |
| WCM9_X1_1 | 7 | 193253 | 26192171 | 0.1248 | 1.227 | 2.3806 | 2.301 | 2 | 2 |
| WCM9_X1_1 | 7 | 26192678 | 158937142 | −0.0096 | 0.8757 | 2.4827 | 1.187 | 2 | 1 |
| WCM9_X1_1 | 8 | 190897 | 146279423 | −0.0112 | 0.8711 | 2.6471 | 1.0112 | 3 | 1 |
| WCM9_X1_1 | 9 | 17066 | 24545256 | −0.1616 | 0.3967 | 2.5618 | 0.0711 | 3 | 0 |
| WCM9_X1_1 | 9 | 24545500 | 134312107 | −0.0174 | 0.8535 | 2.4151 | 1.1986 | 2 | 1 |
| WCM9_X1_1 | 9 | 134314386 | 134393686 | −0.1342 | 0.4917 | NA | NA | NA | NA |
| WCM9_X1_1 | 9 | 134393862 | 141006877 | 0.1689 | 1.3319 | 2.6464 | 2.3883 | 3 | 2 |
| WCM9_X1_1 | 9 | 141008931 | 141111414 | −0.0345 | 0.8042 | NA | NA | NA | NA |
| WCM9_X1_1 | X | 2707709 | 64744479 | 0.3486 | 1.7205 | NA | NA | NA | NA |
| WCM9_X1_1 | X | 64744852 | 75003338 | 0.7277 | 2.4039 | NA | NA | NA | NA |
| WCM9_X1_1 | X | 75003567 | 154509348 | 0.1223 | 1.2211 | NA | NA | NA | NA |
| WCM9_Z3-1 | 1 | 762142 | 59364783 | −0.3745 | −0.6037 | 0.673 | 0.6431 | 1 | 1 |
| WCM9_Z3-1 | 1 | 59787350 | 77987517 | −0.4113 | −0.6982 | 1.1045 | 0.1282 | 1 | 0 |
| WCM9_Z3-1 | 1 | 78024284 | 249212550 | −0.1789 | −0.1538 | 1.0432 | 0.7546 | 1 | 1 |
| WCM9_Z3-1 | 10 | 243934 | 135381766 | −0.0753 | 0.0569 | 1.0402 | 1.0402 | 1 | 1 |
| WCM9_Z3-1 | 11 | 193144 | 134257738 | −0.0527 | 0.1009 | 1.0828 | 1.0621 | 1 | 1 |
| WCM9_Z3-1 | 12 | 283817 | 133810703 | −0.2954 | −0.4122 | 0.762 | 0.741 | 1 | 1 |
| WCM9_Z3-1 | 13 | 19601055 | 115089411 | −0.3174 | −0.4639 | 1.2443 | 0.2058 | 1 | 0 |
| WCM9_Z3-1 | 14 | 20201798 | 65453785 | −0.0075 | 0.187 | 1.1384 | 1.1384 | 1 | 1 |
| WCM9_Z3-1 | 14 | 65471021 | 78028803 | −0.4964 | −0.9331 | 0.8544 | 0.1931 | 1 | 0 |
| WCM9_Z3-1 | 14 | 78036835 | 107283068 | −0.0532 | 0.0998 | 1.0965 | 1.0468 | 1 | 1 |
| WCM9_Z3-1 | 15 | 20170053 | 102389740 | −0.1163 | −0.0246 | 0.993 | 0.9732 | 1 | 1 |
| WCM9_Z3-1 | 16 | 97558 | 90160840 | −0.1476 | −0.0886 | 0.9404 | 0.9404 | 1 | 1 |
| WCM9_Z3-1 | 17 | 131595 | 81083455 | −0.517 | −0.994 | 1.0128 | −0.0086 | 1 | 0 |
| WCM9_Z3-1 | 18 | 48202 | 78005214 | −0.0696 | 0.068 | 1.0544 | 1.0421 | 1 | 1 |
| WCM9_Z3-1 | 19 | 281435 | 59083970 | −0.3677 | −0.5867 | 0.9207 | 0.411 | 1 | 0 |
| WCM9_Z3-1 | 2 | 41620 | 179394802 | −0.1042 | −4.00E−04 | 1.1517 | 0.8478 | 1 | 1 |
| WCM9_Z3-1 | 2 | 179395151 | 179635158 | 0.9601 | 1.6699 | 3.1819 | 3.1819 | 3 | 3 |
| WCM9_Z3-1 | 2 | 179635550 | 242946607 | −0.0354 | 0.134 | 1.0973 | 1.0973 | 1 | 1 |
| WCM9_Z3-1 | 20 | 68375 | 62904932 | −0.1522 | −0.0981 | 1.1199 | 0.7485 | 1 | 1 |
| WCM9_Z3-1 | 21 | 9826203 | 48084226 | −0.244 | −0.295 | 0.8278 | 0.8024 | 1 | 1 |
| WCM9_Z3-1 | 22 | 17060362 | 51214211 | −0.5339 | −1.045 | 0.6836 | 0.2857 | 1 | 0 |
| WCM9_Z3-1 | 3 | 239443 | 54930848 | −0.4193 | −0.7193 | 1.1619 | 0.0528 | 1 | 0 |
| WCM9_Z3-1 | 3 | 54933874 | 148877862 | −0.0052 | 0.1913 | 1.1418 | 1.1418 | 1 | 1 |
| WCM9_Z3-1 | 3 | 148880004 | 151171249 | −0.2709 | −0.3556 | 1.4087 | 0.1544 | 1 | 0 |
| WCM9_Z3-1 | 3 | 151171439 | 196460733 | −0.0814 | 0.0449 | 1.0621 | 1.0011 | 1 | 1 |
| WCM9_Z3-1 | 3 | 196528821 | 197762833 | −0.333 | −0.5013 | NA | NA | NA | NA |
| WCM9_Z3-1 | 4 | 59405 | 190395617 | −0.1563 | −0.1066 | 1.1467 | 0.7108 | 1 | 1 |
| WCM9_Z3-1 | 5 | 140532 | 180687769 | −0.0243 | 0.1553 | 1.1137 | 1.1137 | 1 | 1 |
| WCM9_Z3-1 | 6 | 203516 | 87967574 | −0.0025 | 0.1964 | 1.1458 | 1.1458 | 1 | 1 |
| WCM9_Z3-1 | 6 | 87967696 | 151917713 | −0.4131 | −0.7031 | 1.1679 | 0.0606 | 1 | 0 |
| WCM9_Z3-1 | 6 | 151936677 | 170893438 | −0.1731 | −0.1416 | 0.9198 | 0.8931 | 1 | 1 |
| WCM9_Z3-1 | 7 | 193205 | 158937158 | −0.076 | 0.0554 | 1.0453 | 1.033 | 1 | 1 |
| WCM9_Z3-1 | 8 | 190897 | 444591 | 0.3895 | 0.8606 | NA | NA | NA | NA |
| WCM9_Z3-1 | 8 | 494742 | 47842983 | −0.1523 | −0.0982 | 1.7059 | 0.1625 | 2 | 0 |
| WCM9_Z3-1 | 8 | 48102450 | 146279507 | 0.4347 | 0.9301 | 1.9054 | 1.9054 | 2 | 2 |
| WCM9_Z3-1 | 9 | 214625 | 21409282 | −0.3713 | −0.5957 | 1.0871 | 0.2363 | 1 | 0 |
| WCM9_Z3-1 | 9 | 21409551 | 140995594 | −0.2192 | −0.2404 | 0.8465 | 0.8465 | 1 | 1 |
| WCM9_Z3-1 | 9 | 141000211 | 141071248 | −1.5361 | NA | NA | NA | NA | NA |
| WCM9_Z3-1 | X | 2707688 | 154509348 | −0.1724 | −0.1401 | NA | NA | NA | NA |

(v) Tumor Ploidy and Tumor Content Assessment

To assess tumor ploidy and tumor content (estimation of cfDNA tumor fraction, ctDNA) from plasma DNA samples, an extended version of CLONET was used (Prandi and Demichelis (2019) Curr Protoc Bioinformatics 67:e81), a tool developed to deal with highly heterogeneous tissue samples. It now embeds a refined mathematical approach for local tumor content estimation (TC) (Prandi and Demichelis (2019) Curr Protoc Bioinformatics 67:e81) to further increase the performance in computation time and in the ability to detect somatic aberrations in low TC samples. Specifically, a new method for the calculation of CLONET β values (i.e., the percentage of reads from cells harboring two alleles) was implemented and used in this work. For each segment S, previously identified, the corresponding β value was compute with the following formula:

β=max{i|W(d>Dᵢ)}+(max{i|W(d<Dᵢ)}−min{i|W(d>Dᵢ)})*P

where

P=(median(d)−min(d)/(max(d)−min(d))

and W(d>Dᵢ) is the Mann-Whitney statistics (using significance cutoff at 1%) comparing the mirrored AF distribution distribution of all informative SNPs in segment S (i.e., SNPs with heterozygous genotype in germline patient's sample) and the reference distribution Dᵢ simulating a mono-allelic deletion with β=i(were i∈[0,1]) and AF noise estimated from the germline patient's samples at SNPs positions. The extended CLONET pipeline was applied to estimate TC and ploidy of all cfDNA and tumor tissue samples. CLONET analysis estimated tumor content for 55 out of 69 plasma samples (80% of the total): 30 samples with tumor content >=20% (43% of the total), 43 samples with tumor content >=10% (62% of the total) and 12 samples with tumor content <10%. Manual inspection of the somatic copy number profiles of the remaining 15 samples indicate low or absent tumor signal. Ploidy estimates were available for 68 samples with 12 samples having a ploidy >=2.5. For tumor tissue samples, CLONET analysis estimated tumor content for 90 out of 98 samples: 85 samples with tumor content >=20% (87% of the total), 87 samples with tumor content >=10% and 1 samples with tumor content <10%. Ploidy estimates were available for 96 samples with 34 samples having a ploidy >=2.5. CLONET allele-specific somatic copy number profiles were computed for all plasma and tissue samples at gene-level resolution, using a gene model consisting of 19,027 genes (Table 3).

(vi) Detection of Somatic Single Nucleotide Variants

To identify and characterize somatic single-nucleotide variants (SNVs) in WES captured regions, MuTect (Cibulskis et al. (2013) Nat Biotechnol 31:213-219) was run on BAM files and read duplicates were removed using Picard (available on the World Wide Web at broadinstitute.github.io/picard). Putative somatic SNVs were nominated as genomic positions called by MuTect and having in the plasma/tumor sample a depth of

alternative base, and having in the matched normal sample an allelic fraction <0.01. Oncotator (Ramos et al. (2015) Hum Mutat 36:E2423-2429) was finally used to annotate retained SNVs with variant- and gene-centric information relevant to cancer. Complete annotated list of called missense SNVs is available in Table 4.

TABLE 4
Non-Synonymous SNVs Data

| | | | | | | Allelic | | Allelic | Coverage | | |
|---|---|---|---|---|---|---|---|---|---|---|---|
| | | | | | | fraction | Coverage | Fraction | in | | |
| | | | | | | in | in | in tumor/ | tumor/ | | |
| Chr | Position | Ref. | Alt. | Hugo | Protein | normal | control | plasma | plasma | | |
| (hg19) | (hg19) | base | base | Symbol | Change | sample | sample | sample | sample | Sample | Type |
| 1 | 183093931 | C | A | LAMC1 | p.T856N | 0 | 36 | 0.150685 | 73 | WCM0 | Plasma |
| 1 | 241753378 | C | T | KMO | p.A388V | 0 | 38 | 0.282353 | 85 | WCM0 | Plasma |
| 13 | 48923137 | G | T | RB1 | p.W195C | 0 | 42 | 0.413462 | 104 | WCM0 | Plasma |
| 14 | 38061334 | G | A | FOXA1 | p.R219C | 0 | 31 | 0.223881 | 67 | WCM0 | Plasma |
| 18 | 50918073 | C | A | DCC | p.T835N | 0 | 34 | 0.171429 | 70 | WCM0 | Plasma |
| 19 | 16472591 | G | A | EPS15L1 | p.S862F | 0 | 44 | 0.205882 | 136 | WCM0 | Plasma |
| 3 | 186281967 | C | T | TBCCD1 | p.R51H | 0 | 38 | 0.35 | 100 | WCM0 | Plasma |
| 5 | 121413104 | G | T | LOX | p.P193T | 0 | 47 | 0.097345 | 113 | WCM0 | Plasma |
| 6 | 136681140 | C | G | MAP7 | p.R611S | 0 | 37 | 0.087912 | 91 | WCM0 | Plasma |
| 9 | 131860587 | C | G | CRAT | p.K423N | 0 | 36 | 0.190476 | 63 | WCM0 | Plasma |
| 1 | 145562588 | G | C | ANKRD35 | p.G759A | 0 | 215 | 0.100629 | 159 | WCM0_LM1 | Tissue |
| 11 | 5528985 | A | T | UBQLN3 | p.L602I | 0 | 243 | 0.231707 | 164 | WCM0_LM1 | Tissue |
| 11 | 74904293 | G | A | SLCO2B1 | p.R369H | 0 | 276 | 0.502164 | 231 | WCM0_LM1 | Tissue |
| 1 | 183093931 | C | A | LAMC1 | p.T856N | 0 | 317 | 0.514124 | 354 | WCM0_LM1 | Tissue |
| 11 | 94532860 | C | A | AMOTL1 | p.D168E | 0 | 542 | 0.241791 | 670 | WCM0_LM1 | Tissue |
| 12 | 108685719 | C | G | CMKLR1 | p.D341H | 0 | 304 | 0.563636 | 275 | WCM0_LM1 | Tissue |
| 1 | 241753378 | C | T | KMO | p.A388V | 0 | 232 | 0.411552 | 277 | WCM0_LM1 | Tissue |
| 12 | 6495257 | C | G | LTBR | p.H166Q | 0 | 199 | 0.285714 | 63 | WCM0_LM1 | Tissue |
| 13 | 48923137 | G | T | RB1 | p.W195C | 0 | 266 | 0.875817 | 153 | WCM0_LM1 | Tissue |
| 14 | 38061334 | G | A | FOXA1 | p.R219C | 0 | 560 | 0.427914 | 652 | WCM0_LM1 | Tissue |
| 14 | 93117957 | A | T | RIN3 | p.Q188L | 0 | 271 | 0.261062 | 226 | WCM0_LM1 | Tissue |
| 15 | 78893807 | C | A | CHRNA3 | p.E393* | 0 | 598 | 0.463196 | 557 | WCM0_LM1 | Tissue |
| 17 | 10209870 | T | C | MYH13 | p.E1791G | 0 | 153 | 0.068323 | 161 | WCM0_LM1 | Tissue |
| 17 | 28769407 | A | G | CPD | p.E771G | 0 | 152 | 0.675373 | 268 | WCM0_LM1 | Tissue |
| 1 | 75693493 | A | C | SLC44A5 | p.S301R | 0 | 505 | 0.544029 | 829 | WCM0_LM1 | Tissue |
| 17 | 76130640 | A | T | TMC8 | p.K328* | 0.008696 | 230 | 0.49 | 200 | WCM0_LM1 | Tissue |
| 17 | 80887372 | C | T | TBCD | p.S996L | 0 | 416 | 0.207675 | 443 | WCM0_LM1 | Tissue |
| 18 | 50918073 | C | A | DCC | p.T835N | 0 | 761 | 0.423002 | 513 | WCM0_LM1 | Tissue |
| 19 | 11617019 | G | A | ECSIT | p.Q426* | 0 | 103 | 0.371179 | 229 | WCM0_LM1 | Tissue |
| 19 | 16040379 | C | G | CYP4F11 | p.L77F | 0 | 344 | 0.307818 | 614 | WCM0_LM1 | Tissue |
| 19 | 16472591 | G | A | EPS15L1 | p.S862F | 0 | 90 | 0.451977 | 177 | WCM0_LM1 | Tissue |
| 19 | 19313184 | G | A | NR2C2AP | p.H87Y | 0 | 318 | 0.272861 | 678 | WCM0_LM1 | Tissue |
| 19 | 22847655 | G | A | ZNF492 | p.C395Y | 0 | 44 | 0.122222 | 90 | WCM0_LM1 | Tissue |
| 1 | 9305367 | A | C | H6PD | p.E125A | 0 | 604 | 0.311178 | 331 | WCM0_LM1 | Tissue |
| 19 | 40380382 | C | A | FCGBP | p.G3645C | 0 | 33 | 0.090909 | 33 | WCM0_LM1 | Tissue |
| 19 | 48643373 | G | C | LIG1 | p.N314K | 0 | 77 | 0.1375 | 80 | WCM0_LM1 | Tissue |
| 19 | 50029305 | C | G | FCGRT | p.L343V | 0 | 976 | 0.054455 | 606 | WCM0_LM1 | Tissue |
| 19 | 51189522 | T | A | SHANK1 | p.H850L | 0 | 453 | 0.287781 | 622 | WCM0_LM1 | Tissue |
| 19 | 56370147 | T | G | NLRP4 | p.F463C | 0 | 547 | 0.121437 | 807 | WCM0_LM1 | Tissue |
| 19 | 5843818 | G | T | FUT3 | p.Q345K | 0 | 37 | 0.0625 | 48 | WCM0_LM1 | Tissue |
| 20 | 47865195 | G | A | ZNFX1 | p.R1456C | 0 | 493 | 0.514477 | 449 | WCM0_LM1 | Tissue |
| 20 | 60882854 | G | A | ADRM1 | p.V276I | 0 | 474 | 0.482684 | 462 | WCM0_LM1 | Tissue |
| 22 | 21153533 | G | A | PI4KA | p.R560* | 0 | 102 | 0.097561 | 41 | WCM0_LM1 | Tissue |
| 3 | 186281967 | C | T | TBCCD1 | p.R51H | 0 | 368 | 0.486216 | 399 | WCM0_LM1 | Tissue |
| 4 | 134073066 | C | T | PCDH10 | p.P591S | 0 | 428 | 0.101504 | 532 | WCM0_LM1 | Tissue |
| 5 | 121413104 | G | T | LOX | p.P193T | 0 | 533 | 0.232782 | 726 | WCM0_LM1 | Tissue |
| 6 | 108492701 | G | A | NR2E1 | p.R22H | 0 | 182 | 0.431373 | 153 | WCM0_LM1 | Tissue |
| 6 | 110567235 | A | G | METTL24 | p.S339P | 0.001751 | 571 | 0.506156 | 731 | WCM0_LM1 | Tissue |
| 6 | 136681140 | C | G | MAP7 | p.R611S | 0 | 311 | 0.216912 | 272 | WCM0_LM1 | Tissue |
| 6 | 24716433 | C | G | C6orf62 | p.E83D | 0 | 231 | 0.193676 | 253 | WCM0_LM1 | Tissue |
| 7 | 141619105 | G | T | OR9A4 | p.V144L | 0 | 493 | 0.48595 | 605 | WCM0_LM1 | Tissue |
| 7 | 142574254 | G | C | TRPV6 | p.D223E | 0 | 350 | 0.152542 | 413 | WCM0_LM1 | Tissue |
| 8 | 22165564 | G | A | PIWIL2 | p.R555H | 0 | 74 | 0.915254 | 59 | WCM0_LM1 | Tissue |
| 9 | 131860587 | C | G | CRAT | p.K423N | 0 | 365 | 0.247387 | 287 | WCM0_LM1 | Tissue |
| 9 | 135139691 | A | G | SETX | p.S2657P | 0.001193 | 838 | 0.056805 | 845 | WCM0_LM1 | Tissue |
| 9 | 139902948 | C | A | ABCA2 | p.E2398* | 0 | 251 | 0.25 | 192 | WCM0_LM1 | Tissue |
| X | 135291473 | C | T | FHL1 | p.R254C | 0 | 31 | 0.979592 | 49 | WCM0_LM1 | Tissue |
| X | 96369830 | C | T | DIAPH2 | p.L819F | 0 | 282 | 0.465046 | 329 | WCM0_LM1 | Tissue |
| 11 | 119170354 | G | A | CBL | p.E862K | 0 | 631 | 0.083086 | 674 | WCM0_LM2 | Tissue |
| 11 | 26725464 | C | A | SLC5A12 | p.A186S | 0 | 88 | 0.067568 | 74 | WCM0_LM2 | Tissue |
| 1 | 145562588 | G | C | ANKRD35 | p.G759A | 0 | 215 | 0.156463 | 147 | WCM0_LM2 | Tissue |
| 11 | 4615558 | G | T | OR52I1 | p.S97I | 0 | 81 | 0.071429 | 42 | WCM0_LM2 | Tissue |
| 11 | 6024189 | C | A | OR56A4 | p.V64F | 0 | 176 | 0.050505 | 99 | WCM0_LM2 | Tissue |
| 11 | 74904293 | G | A | SLCO2B1 | p.R369H | 0 | 276 | 0.526923 | 260 | WCM0_LM2 | Tissue |
| 1 | 183093931 | C | A | LAMC1 | p.T856N | 0 | 317 | 0.334694 | 245 | WCM0_LM2 | Tissue |
| 11 | 94532860 | C | A | AMOTL1 | p.D168E | 0 | 542 | 0.239598 | 697 | WCM0_LM2 | Tissue |
| 12 | 108685719 | C | G | CMKLR1 | p.D341H | 0 | 304 | 0.508929 | 224 | WCM0_LM2 | Tissue |
| 1 | 241753378 | C | T | KMO | p.A388V | 0 | 232 | 0.336788 | 193 | WCM0_LM2 | Tissue |
| 13 | 48923137 | G | T | RB1 | p.W195C | 0 | 266 | 0.9375 | 112 | WCM0_LM2 | Tissue |
| 14 | 38061334 | G | A | FOXA1 | p.R219C | 0 | 560 | 0.503949 | 633 | WCM0_LM2 | Tissue |
| 14 | 93117957 | A | T | RIN3 | p.Q188L | 0 | 271 | 0.252033 | 246 | WCM0_LM2 | Tissue |
| 15 | 78893807 | C | A | CHRNA3 | p.E393* | 0 | 598 | 0.486957 | 575 | WCM0_LM2 | Tissue |
| 16 | 29755841 | C | A | C16orf54 | p.E144D | 0 | 98 | 0.067797 | 59 | WCM0_LM2 | Tissue |
| 16 | 68056205 | C | T | DDX28 | p.D301N | 0 | 663 | 0.052326 | 344 | WCM0_LM2 | Tissue |
| 17 | 28769407 | A | G | CPD | p.E771G | 0 | 152 | 0.692661 | 218 | WCM0_LM2 | Tissue |
| 1 | 75693493 | A | C | SLC44A5 | p.S301R | 0 | 505 | 0.496656 | 598 | WCM0_LM2 | Tissue |
| 17 | 57153068 | C | A | TRIM37 | p.K208N | 0 | 116 | 0.051948 | 77 | WCM0_LM2 | Tissue |
| 17 | 76130640 | A | T | TMC8 | p.K328* | 0.008696 | 230 | 0.471264 | 174 | WCM0_LM2 | Tissue |
| 17 | 80887372 | C | T | TBCD | p.S996L | 0 | 416 | 0.237179 | 468 | WCM0_LM2 | Tissue |
| 18 | 50918073 | C | A | DCC | p.T835N | 0 | 761 | 0.424036 | 441 | WCM0_LM2 | Tissue |
| 19 | 11617019 | G | A | ECSIT | p.Q426* | 0 | 103 | 0.252747 | 182 | WCM0_LM2 | Tissue |
| 19 | 16040379 | C | G | CYP4F11 | p.L77F | 0 | 344 | 0.283582 | 536 | WCM0_LM2 | Tissue |
| 19 | 16472591 | G | A | EPS15L1 | p.S862F | 0 | 90 | 0.341346 | 208 | WCM0_LM2 | Tissue |
| 19 | 19313184 | G | A | NR2C2AP | p.H87Y | 0 | 318 | 0.225962 | 624 | WCM0_LM2 | Tissue |
| 19 | 22847655 | G | A | ZNF492 | p.C395Y | 0 | 44 | 0.255319 | 47 | WCM0_LM2 | Tissue |
| 1 | 9305367 | A | C | H6PD | p.E125A | 0 | 604 | 0.267956 | 362 | WCM0_LM2 | Tissue |
| 19 | 48643373 | G | C | LIG1 | p.N314K | 0 | 77 | 0.146667 | 75 | WCM0_LM2 | Tissue |
| 19 | 51189522 | T | A | SHANK1 | p.H850L | 0 | 453 | 0.342908 | 557 | WCM0_LM2 | Tissue |
| 19 | 56370147 | T | G | NLRP4 | p.F463C | 0 | 547 | 0.178344 | 628 | WCM0_LM2 | Tissue |
| 19 | 5843871 | C | T | FUT3 | p.R327Q | 0 | 39 | 0.069767 | 43 | WCM0_LM2 | Tissue |
| 20 | 47865195 | G | A | ZNFX1 | p.R1456C | 0 | 493 | 0.488281 | 512 | WCM0_LM2 | Tissue |
| 20 | 60882854 | G | A | ADRM1 | p.V276I | 0 | 474 | 0.505929 | 506 | WCM0_LM2 | Tissue |
| 2 | 131415089 | G | T | POTEJ | p.R919L | 0 | 91 | 0.054348 | 92 | WCM0_LM2 | Tissue |
| 3 | 186281967 | C | T | TBCCD1 | p.R51H | 0 | 368 | 0.428571 | 427 | WCM0_LM2 | Tissue |
| 5 | 121413104 | G | T | LOX | p.P193T | 0 | 533 | 0.250382 | 655 | WCM0_LM2 | Tissue |
| 6 | 108492701 | G | A | NR2E1 | p.R22H | 0 | 182 | 0.536585 | 164 | WCM0_LM2 | Tissue |
| 6 | 110567235 | A | G | METTL24 | p.S339P | 0.001751 | 571 | 0.484892 | 695 | WCM0_LM2 | Tissue |
| 6 | 136681140 | C | G | MAP7 | p.R611S | 0 | 311 | 0.291304 | 230 | WCM0_LM2 | Tissue |
| 6 | 24716433 | C | G | C6orf62 | p.E83D | 0 | 231 | 0.263374 | 243 | WCM0_LM2 | Tissue |
| 6 | 32012994 | C | G | TNXB | p.M3572I | 0 | 44 | 0.097561 | 41 | WCM0_LM2 | Tissue |
| 7 | 141619105 | G | T | OR9A4 | p.V144L | 0 | 493 | 0.484603 | 617 | WCM0_LM2 | Tissue |
| 7 | 150674998 | G | A | KCNH2 | p.P2S | 0 | 49 | 0.08 | 50 | WCM0_LM2 | Tissue |
| 7 | 4876021 | G | C | RADIL | p.L251V | 0 | 43 | 0.071429 | 42 | WCM0_LM2 | Tissue |
| 8 | 22165564 | G | A | PIWIL2 | p.R555H | 0 | 74 | 1 | 38 | WCM0_LM2 | Tissue |
| 9 | 131860587 | C | G | CRAT | p.K423N | 0 | 365 | 0.212963 | 324 | WCM0_LM2 | Tissue |
| 9 | 139902948 | C | A | ABCA2 | p.E2398* | 0 | 251 | 0.220149 | 268 | WCM0_LM2 | Tissue |
| X | 135291473 | C | T | FHL1 | p.R254C | 0 | 31 | 0.96875 | 64 | WCM0_LM2 | Tissue |
| X | 96369830 | C | T | DIAPH2 | p.L819F | 0 | 282 | 0.474903 | 259 | WCM0_LM2 | Tissue |
| 10 | 31134360 | C | T | ZNF438 | p.E673K | 0 | 965 | 0.43131 | 626 | WCMO_PM1 | Tissue |
| 10 | 89118177 | C | A | NUTM2D | p.A52D | 0 | 55 | 0.06383 | 47 | WCMO_PM1 | Tissue |
| 11 | 55890099 | C | A | OR8H3 | p.A84E | 0 | 76 | 0.058824 | 51 | WCMO_PM1 | Tissue |
| 1 | 156012692 | C | A | UBQLN4 | p.S380I | 0 | 142 | 0.05618 | 89 | WCMO_PM1 | Tissue |
| 11 | 74904293 | G | A | SLCO2B1 | p.R369H | 0 | 276 | 0.430303 | 330 | WCMO_PM1 | Tissue |
| 1 | 241753378 | C | T | KMO | p.A388V | 0 | 232 | 0.422311 | 251 | WCMO_PM1 | Tissue |
| 13 | 48923137 | G | T | RB1 | p.W195C | 0 | 266 | 0.944444 | 126 | WCMO_PM1 | Tissue |
| 14 | 38061334 | G | A | FOXA1 | p.R219C | 0 | 560 | 0.623639 | 643 | WCMO_PM1 | Tissue |
| 1 | 46105974 | C | A | GPBP1L1 | p.G218* | 0 | 253 | 0.20649 | 339 | WCMO_PM1 | Tissue |
| 15 | 78893807 | C | A | CHRNA3 | p.E393* | 0 | 598 | 0.333333 | 456 | WCMO_PM1 | Tissue |
| 17 | 10558268 | G | C | MYH3 | p.C38W | 0 | 1106 | 0.20547 | 1426 | WCMO_PM1 | Tissue |
| 17 | 10558269 | C | T | MYH3 | p.C38Y | 0 | 1095 | 0.20736 | 1413 | WCMO_PM1 | Tissue |
| 17 | 28769407 | A | G | CPD | p.E771G | 0 | 152 | 0.61674 | 227 | WCMO_PM1 | Tissue |
| 1 | 75693493 | A | C | SLC44A5 | p.S301R | 0 | 505 | 0.46849 | 841 | WCMO_PM1 | Tissue |
| 17 | 76130640 | A | T | TMC8 | p.K328* | 0.008696 | 230 | 0.477387 | 199 | WCMO_PM1 | Tissue |
| 1 | 92801974 | C | G | RPAP2 | p.R537G | 0 | 163 | 0.455556 | 90 | WCM0_PM1 | Tissue |
| 19 | 51189522 | T | A | SHANK1 | p.H850L | 0 | 453 | 0.44086 | 465 | WCM0_PM1 | Tissue |
| 20 | 47865195 | G | A | ZNFX1 | p.R1456C | 0 | 493 | 0.539286 | 560 | WCM0_PM1 | Tissue |
| 20 | 60882854 | G | A | ADRM1 | p.V276I | 0 | 474 | 0.483146 | 623 | WCM0_PM1 | Tissue |
| 21 | 45993661 | G | T | KRTAP10-4 | p.S9I | 0 | 74 | 0.072727 | 55 | WCM0_PM1 | Tissue |
| 3 | 186281967 | C | T | TBCCD1 | p.R51H | 0 | 368 | 0.458774 | 473 | WCM0_PM1 | Tissue |
| 3 | 58121847 | C | G | FLNB | p.R1605G | 0 | 460 | 0.083004 | 253 | WCM0_PM1 | Tissue |
| 6 | 108492701 | G | A | NR2E1 | p.R22H | 0 | 182 | 0.440171 | 234 | WCM0_PM1 | Tissue |
| 6 | 110567235 | A | G | METTL24 | p.S339P | 0.001751 | 571 | 0.469697 | 792 | WCM0_PM1 | Tissue |
| 6 | 136681140 | C | G | MAP7 | p.R611S | 0 | 311 | 0.206704 | 358 | WCM0_PM1 | Tissue |
| 7 | 141619105 | G | T | OR9A4 | p.V144L | 0 | 493 | 0.491329 | 692 | WCM0_PM1 | Tissue |
| 8 | 121823950 | G | C | SNTB1 | p.A45G | 0 | 113 | 0.25641 | 117 | WCM0_PM1 | Tissue |
| 8 | 121823951 | C | T | SNTB1 | p.A45T | 0.008929 | 112 | 0.25641 | 117 | WCM0_PM1 | Tissue |
| 8 | 22165564 | G | A | PIWIL2 | p.R555H | 0 | 74 | 0.934426 | 61 | WCM0_PM1 | Tissue |
| X | 135291473 | C | T | FHL1 | p.R254C | 0 | 31 | 0.964286 | 56 | WCM0_PM1 | Tissue |
| 10 | 31134360 | C | T | ZNF438 | p.E673K | 0 | 965 | 0.503425 | 584 | WCM0_PM2 | Tissue |
| 11 | 64119018 | G | T | CCDC88B | p.S1010I | 0 | 81 | 0.066667 | 45 | WCM0_PM2 | Tissue |
| 11 | 74904293 | G | A | SLCO2B1 | p.R369H | 0 | 276 | 0.53125 | 224 | WCM0_PM2 | Tissue |
| 1 | 241753378 | C | T | KMO | p.A388V | 0 | 232 | 0.495763 | 236 | WCM0_PM2 | Tissue |
| 13 | 48923137 | G | T | RB1 | p.W195C | 0 | 266 | 0.96988 | 166 | WCM0_PM2 | Tissue |
| 1 | 36642105 | G | A | MAP7D1 | p.G386S | 0.008333 | 120 | 0.058252 | 103 | WCM0_PM2 | Tissue |
| 14 | 38061334 | G | A | FOXA1 | p.R219C | 0 | 560 | 0.705769 | 520 | WCM0_PM2 | Tissue |
| 15 | 78893807 | C | A | CHRNA3 | p.E393* | 0 | 598 | 0.291284 | 436 | WCM0_PM2 | Tissue |
| 17 | 10558268 | G | C | MYH3 | p.C38W | 0 | 1106 | 0.220508 | 1102 | WCM0_PM2 | Tissue |
| 17 | 10558269 | C | T | MYH3 | p.C38Y | 0 | 1095 | 0.224565 | 1091 | WCM0_PM2 | Tissue |
| 17 | 28769407 | A | G | CPD | p.E771G | 0 | 152 | 0.618257 | 241 | WCM0_PM2 | Tissue |
| 17 | 39383010 | C | A | KRTAP9-2 | p.T35K | 0 | 57 | 0.078947 | 38 | WCM0_PM2 | Tissue |
| 17 | 39389012 | G | A | KRTAP9-3 | p.G87R | 0 | 71 | 0.081967 | 61 | WCM0_PM2 | Tissue |
| 1 | 75693493 | A | C | SLC44A5 | p.S301R | 0 | 505 | 0.489388 | 801 | WCM0_PM2 | Tissue |
| 17 | 57941135 | C | A | TUBD1 | p.Q383H | 0 | 185 | 0.064103 | 78 | WCM0_PM2 | Tissue |
| 17 | 76130640 | A | T | TMC8 | p.K328* | 0.008696 | 230 | 0.469072 | 194 | WCM0_PM2 | Tissue |
| 18 | 29340528 | G | T | SLC25A52 | p.H33N | 0 | 36 | 0.068182 | 44 | WCM0_PM2 | Tissue |
| 1 | 92801974 | C | G | RPAP2 | p.R537G | 0 | 163 | 0.44 | 100 | WCM0_PM2 | Tissue |
| 19 | 41383141 | C | T | CYP2A7 | p.R372H | 0 | 303 | 0.126761 | 355 | WCM0_PM2 | Tissue |
| 19 | 51189522 | T | A | SHANK1 | p.H850L | 0 | 453 | 0.421659 | 434 | WCM0_PM2 | Tissue |
| 20 | 47865195 | G | A | ZNFX1 | p.R1456C | 0 | 493 | 0.474041 | 443 | WCM0_PM2 | Tissue |
| 20 | 60882854 | G | A | ADRM1 | p.V276I | 0 | 474 | 0.469828 | 464 | WCM0_PM2 | Tissue |
| 3 | 186281967 | C | T | TBCCD1 | p.R51H | 0 | 368 | 0.498818 | 423 | WCM0_PM2 | Tissue |
| 6 | 108492701 | G | A | NR2E1 | p.R22H | 0 | 182 | 0.407609 | 184 | WCM0_PM2 | Tissue |
| 6 | 110567235 | A | G | METTL24 | p.S339P | 0.001751 | 571 | 0.491803 | 671 | WCM0_PM2 | Tissue |
| 6 | 136681140 | C | G | MAP7 | p.R611S | 0 | 311 | 0.266106 | 357 | WCM0_PM2 | Tissue |
| 7 | 141619105 | G | T | OR9A4 | p.V144L | 0 | 493 | 0.463636 | 660 | WCM0_PM2 | Tissue |
| 7 | 38433732 | G | A | AMPH | p.A494V | 0 | 628 | 0.079893 | 751 | WCM0_PM2 | Tissue |
| 8 | 22165564 | G | A | PIWIL2 | p.R555H | 0 | 74 | 0.970588 | 34 | WCM0_PM2 | Tissue |
| 8 | 42940369 | T | A | FNTA | p.S362T | 0 | 94 | 0.082645 | 121 | WCM0_PM2 | Tissue |
| X | 135291473 | C | T | FHL1 | p.R254C | 0 | 31 | 0.974359 | 39 | WCM0_PM2 | Tissue |
| 10 | 31134360 | C | T | ZNF438 | p.E673K | 0 | 965 | 0.279528 | 762 | WCM0_T24 | Tissue |
| 11 | 105923958 | C | T | KBTBD3 | p.W486* | 0 | 313 | 0.052036 | 442 | WCM0_T24 | Tissue |
| 11 | 74904293 | G | A | SLCO2B1 | p.R369H | 0 | 276 | 0.523466 | 277 | WCM0_T24 | Tissue |
| 12 | 110290404 | C | A | GLTP | p.E196* | 0 | 67 | 0.09375 | 32 | WCM0_T24 | Tissue |
| 1 | 241753378 | C | T | KMO | p.A388V | 0 | 232 | 0.434164 | 281 | WCM0_T24 | Tissue |
| 13 | 48923137 | G | T | RB1 | p.W195C | 0 | 266 | 0.84492 | 187 | WCM0_T24 | Tissue |
| 14 | 38061334 | G | A | FOXA1 | p.R219C | 0 | 560 | 0.545455 | 649 | WCM0_T24 | Tissue |
| 15 | 101605608 | C | A | LRRK1 | p.P1656T | 0 | 432 | 0.079186 | 442 | WCM0_T24 | Tissue |
| 15 | 78893807 | C | A | CHRNA3 | p.E393* | 0 | 598 | 0.344231 | 520 | WCM0_T24 | Tissue |
| 17 | 10558268 | G | C | MYH3 | p.C38W | 0 | 1106 | 0.154589 | 1242 | WCM0_T24 | Tissue |
| 17 | 10558269 | C | T | MYH3 | p.C38Y | 0 | 1095 | 0.15154 | 1234 | WCM0_T24 | Tissue |
| 17 | 11515065 | C | G | DNAH9 | p.A291G | 0 | 120 | 0.074074 | 135 | WCM0_T24 | Tissue |
| 17 | 28769407 | A | G | CPD | p.E771G | 0 | 152 | 0.600823 | 243 | WCM0_T24 | Tissue |
| 17 | 39778650 | T | C | KRT17 | p.N210S | 0 | 52 | 0.06383 | 47 | WCM0_T24 | Tissue |
| 1 | 75693493 | A | C | SLC44A5 | p.S301R | 0 | 505 | 0.45814 | 860 | WCM0_T24 | Tissue |
| 17 | 76130640 | A | T | TMC8 | p.K328* | 0.008696 | 230 | 0.478448 | 232 | WCM0_T24 | Tissue |
| 1 | 92801974 | C | G | RPAP2 | p.R537G | 0 | 163 | 0.21875 | 96 | WCM0_T24 | Tissue |
| 19 | 51189522 | T | A | SHANK1 | p.H850L | 0 | 453 | 0.380457 | 481 | WCM0_T24 | Tissue |
| 20 | 42969923 | G | A | R3HDML | p.V117M | 0 | 504 | 0.083164 | 493 | WCM0_T24 | Tissue |
| 20 | 47865195 | G | A | ZNFX1 | p.R1456C | 0 | 493 | 0.489362 | 517 | WCM0_T24 | Tissue |
| 20 | 60882854 | G | A | ADRM1 | p.V276I | 0 | 474 | 0.474766 | 535 | WCM0_T24 | Tissue |
| 2 | 103125390 | C | T | SLC9A4 | p.R496C | 0 | 122 | 0.12987 | 154 | WCM0_T24 | Tissue |
| 2 | 128471433 | T | A | WDR33 | p.D1011V | 0 | 711 | 0.231183 | 372 | WCM0_T24 | Tissue |
| 21 | 46117138 | C | A | KRTAP10-12 | p.L8M | 0 | 59 | 0.055556 | 54 | WCM0_T24 | Tissue |
| 3 | 186281967 | C | T | TBCCD1 | p.R51H | 0 | 368 | 0.39759 | 415 | WCM0_T24 | Tissue |
| 3 | 23960890 | C | A | RPL15 | p.S171R | 0 | 86 | 0.056604 | 53 | WCM0_T24 | Tissue |
| 3 | 50338477 | G | A | HYAL1 | p.A311V | 0 | 119 | 0.055556 | 54 | WCM0_T24 | Tissue |
| 3 | 52817058 | C | T | ITIH1 | p.S339L | 0 | 461 | 0.12 | 350 | WCM0_T24 | Tissue |
| 6 | 108492701 | G | A | NR2E1 | p.R22H | 0 | 182 | 0.432692 | 208 | WCM0_T24 | Tissue |
| 6 | 110567235 | A | G | METTL24 | p.S339P | 0.001751 | 571 | 0.485577 | 832 | WCM0_T24 | Tissue |
| 6 | 136681140 | C | G | MAP7 | p.R611S | 0 | 311 | 0.22963 | 405 | WCM0_T24 | Tissue |
| 6 | 143486255 | G | A | AIG1 | p.D112N | 0 | 296 | 0.054054 | 407 | WCM0_T24 | Tissue |
| 6 | 34499489 | G | A | PACSIN1 | p.D384N | 0 | 247 | 0.099631 | 271 | WCM0_T24 | Tissue |
| 7 | 141619105 | G | T | OR9A4 | p.V144L | 0 | 493 | 0.472477 | 654 | WCM0_T24 | Tissue |
| 8 | 22165564 | G | A | PIWIL2 | p.R555H | 0 | 74 | 0.88 | 50 | WCM0_T24 | Tissue |
| 9 | 73027843 | G | A | KLF9 | p.P146L | 0 | 283 | 0.056522 | 230 | WCM0_T24 | Tissue |
| X | 135291473 | C | T | FHL1 | p.R254C | 0 | 31 | 0.883721 | 43 | WCM0_T24 | Tissue |
| 10 | 23257326 | G | T | ARMC3 | p.G275V | 0 | 161 | 0.338983 | 59 | WCM0_T5 | Tissue |
| 10 | 92616897 | T | A | HTR7 | p.I178F | 0 | 367 | 0.163934 | 244 | WCM0_T5 | Tissue |
| 1 | 117122376 | T | C | IGSF3 | p.Y991C | 0 | 692 | 0.133772 | 456 | WCM0_T5 | Tissue |
| 11 | 74904293 | G | A | SLCO2B1 | p.R369H | 0 | 276 | 0.439331 | 239 | WCM0_T5 | Tissue |
| 1 | 220863884 | C | T | C1orf115 | p.A47V | 0 | 85 | 0.196078 | 51 | WCM0_T5 | Tissue |
| 1 | 241753378 | C | T | KMO | p.A388V | 0 | 232 | 0.412451 | 257 | WCM0_T5 | Tissue |
| 13 | 48923137 | G | T | RB1 | p.W195C | 0 | 266 | 0.948718 | 156 | WCM0_T5 | Tissue |
| 14 | 24644791 | G | T | REC8 | p.E168* | 0.001883 | 531 | 0.227273 | 1386 | WCM0_T5 | Tissue |
| 14 | 38061334 | G | A | FOXA1 | p.R219C | 0 | 560 | 0.601887 | 530 | WCM0_T5 | Tissue |
| 15 | 78893807 | C | A | CHRNA3 | p.E393* | 0 | 598 | 0.487319 | 552 | WCM0_T5 | Tissue |
| 16 | 65005535 | C | G | CDH11 | p.S530T | 0 | 308 | 0.488987 | 227 | WCM0_T5 | Tissue |
| 16 | 681269 | C | G | WFIKKN1 | p.P6A | 0 | 220 | 0.066667 | 75 | WCM0_T5 | Tissue |
| 17 | 18566133 | G | T | ZNF286B | p.S229* | 0 | 36 | 0.085714 | 35 | WCM0_T5 | Tissue |
| 17 | 28769407 | A | G | CPD | p.E771G | 0 | 152 | 0.65 | 260 | WCM0_T5 | Tissue |
| 1 | 75693493 | A | C | SLC44A5 | p.S301R | 0 | 505 | 0.492976 | 783 | WCM0_T5 | Tissue |
| 17 | 76130640 | A | T | TMC8 | p.K328* | 0.008696 | 230 | 0.49162 | 179 | WCM0_T5 | Tissue |
| 19 | 51189522 | T | A | SHANK1 | p.H850L | 0 | 453 | 0.419565 | 460 | WCM0_T5 | Tissue |
| 20 | 47865195 | G | A | ZNFX1 | p.R1456C | 0 | 493 | 0.533835 | 399 | WCM0_T5 | Tissue |
| 20 | 60882854 | G | A | ADRM1 | p.V276I | 0 | 474 | 0.521415 | 537 | WCM0_T5 | Tissue |
| 2 | 110372320 | A | C | SOWAHC | p.E85A | 0 | 215 | 0.08642 | 162 | WCM0_T5 | Tissue |
| 3 | 186281967 | C | T | TBCCD1 | p.R51H | 0 | 368 | 0.414286 | 350 | WCM0_T5 | Tissue |
| 4 | 160268015 | C | A | RAPGEF2 | p.Q1032K | 0 | 470 | 0.116114 | 422 | WCM0_T5 | Tissue |
| 4 | 57367953 | A | C | SRP72 | p.K648Q | 0 | 195 | 0.097701 | 174 | WCM0_T5 | Tissue |
| 5 | 136973004 | C | A | KLHL3 | p.V434L | 0 | 113 | 0.168919 | 148 | WCM0_T5 | Tissue |
| 5 | 148427526 | T | A | SH3TC2 | p.S60C | 0 | 72 | 0.132743 | 113 | WCM0_T5 | Tissue |
| 5 | 21760693 | G | T | CDH12 | p.P536Q | 0 | 159 | 0.164894 | 188 | WCM0_T5 | Tissue |
| 5 | 40945369 | A | G | C7 | p.T213A | 0 | 342 | 0.1125 | 400 | WCM0_T5 | Tissue |
| 6 | 108492701 | G | A | NR2E1 | p.R22H | 0 | 182 | 0.648438 | 128 | WCM0_T5 | Tissue |
| 6 | 110567235 | A | G | METTL24 | p.S339P | 0.001751 | 571 | 0.309122 | 592 | WCM0_T5 | Tissue |
| 7 | 141619105 | G | T | OR9A4 | p.V144L | 0 | 493 | 0.519947 | 752 | WCM0_T5 | Tissue |
| 8 | 22165564 | G | A | PIWIL2 | p.R555H | 0 | 74 | 0.979592 | 49 | WCM0_T5 | Tissue |
| X | 117577634 | C | G | WDR44 | p.F832L | 0 | 99 | 0.050505 | 99 | WCM0_T5 | Tissue |
| X | 135291473 | C | T | FHL1 | p.R254C | 0 | 31 | 0.970588 | 34 | WCM0_T5 | Tissue |
| X | 26157715 | G | C | MAGEB18 | p.G205R | 0 | 106 | 0.180451 | 133 | WCM0_T5 | Tissue |
| X | 37026700 | C | T | FAM47C | p.R73C | 0 | 66 | 0.481013 | 79 | WCM0_T5 | Tissue |
| 10 | 47669242 | C | T | ANTXRL | p.T247M | 0 | 40 | 0.135593 | 59 | WCM1 | Plasma |
| 10 | 5139715 | T | A | AKR1C3 | p.Y114* | 0 | 64 | 0.104651 | 86 | WCM1 | Plasma |
| 1 | 12008048 | T | A | PLOD1 | p.L31H | 0 | 59 | 0.267442 | 86 | WCM1 | Plasma |
| 13 | 58208319 | G | T | PCDH17 | p.A547S | 0 | 41 | 0.209302 | 43 | WCM1 | Plasma |
| 15 | 28412883 | C | A | HERC2 | p.D3502Y | 0 | 54 | 0.177215 | 79 | WCM1 | Plasma |
| 15 | 93563245 | G | T | CHD2 | p.R1637L | 0 | 58 | 0.0625 | 64 | WCM1 | Plasma |
| 16 | 58557296 | C | A | CNOT1 | p.A2299S | 0 | 65 | 0.073171 | 82 | WCM1 | Plasma |
| 1 | 70728464 | T | C | ANKRD13C | p.D521G | 0 | 33 | 0.061538 | 65 | WCM1 | Plasma |
| 17 | 19318461 | G | A | RNF112 | p.A413T | 0 | 45 | 0.122449 | 49 | WCM1 | Plasma |
| 17 | 72588241 | C | T | C17orf77 | p.A19V | 0 | 32 | 0.113924 | 79 | WCM1 | Plasma |
| 18 | 28923489 | A | C | DSG1 | p.E588D | 0 | 70 | 0.219298 | 114 | WCM1 | Plasma |
| 20 | 36625323 | G | T | TTI1 | p.D942E | 0 | 49 | 0.059701 | 67 | WCM1 | Plasma |
| 2 | 187523882 | C | T | ITGAV | p.P613S | 0 | 55 | 0.223881 | 67 | WCM1 | Plasma |
| 4 | 164066996 | T | G | NAF1 | p.N219H | 0 | 89 | 0.163934 | 122 | WCM1 | Plasma |
| 4 | 55152038 | G | A | PDGFRA | p.V824I | 0 | 56 | 0.239437 | 71 | WCM1 | Plasma |
| 4 | 57244443 | G | T | AASDH | p.A180E | 0 | 55 | 0.112903 | 62 | WCM1 | Plasma |
| 5 | 5486894 | C | T | KIAA0947 | p.S2194L | 0 | 78 | 0.201835 | 109 | WCM1 | Plasma |
| 7 | 117359625 | T | C | CTTNBP2 | p.D1526G | 0 | 43 | 0.129412 | 85 | WCM1 | Plasma |
| 7 | 128409148 | A | G | CALU | p.D292G | 0 | 55 | 0.153846 | 78 | WCM1 | Plasma |
| 8 | 125115401 | A | T | FER1L6 | p.N1714Y | 0 | 60 | 0.136842 | 95 | WCM1 | Plasma |
| 8 | 95142932 | G | A | CDH17 | p.R774W | 0 | 60 | 0.233645 | 107 | WCM1 | Plasma |
| 9 | 14155884 | C | T | NFIB | p.E209K | 0 | 69 | 0.229167 | 96 | WCM1 | Plasma |
| X | 144904175 | T | C | SLITRK2 | p.Y78H | 0 | 43 | 0.202532 | 79 | WCM1 | Plasma |
| 15 | 43748441 | A | C | TP53BP1 | p.S784A | 0 | 35 | 0.055556 | 72 | WCM10 | Plasma |
| 16 | 58608985 | G | T | CNOT1 | p.L585I | 0 | 67 | 0.056338 | 71 | WCM10 | Plasma |
| 10 | 102782018 | C | A | PDZD7 | p.G223C | 0 | 237 | 0.284916 | 179 | WCM10_Z1-1 | Tissue |
| 10 | 105128125 | G | A | TAF5 | p.V127M | 0 | 88 | 0.092105 | 76 | WCM10_Z1-1 | Tissue |
| 10 | 105194032 | C | T | PDCD11 | p.R1198W | 0 | 151 | 0.354839 | 124 | WCM10_Z1-1 | Tissue |
| 10 | 105207219 | G | A | CALHM2 | p.A221V | 0 | 100 | 0.253165 | 79 | WCM10_Z1-1 | Tissue |
| 10 | 105331300 | G | A | NEURL | p.G124S | 0 | 141 | 0.318182 | 132 | WCM10_Z1-1 | Tissue |
| 10 | 105831807 | G | A | COL17A1 | p.A149V | 0 | 308 | 0.326923 | 312 | WCM10_Z1-1 | Tissue |
| 10 | 105889913 | G | A | WDR96 | p.A1661V | 0 | 178 | 0.058824 | 170 | WCM10_Z1-1 | Tissue |
| 10 | 115612923 | C | T | DCLRE1A | p.E7K | 0 | 79 | 0.314286 | 105 | WCM10_Z1-1 | Tissue |
| 10 | 118443350 | C | T | HSPA12A | p.V263I | 0 | 81 | 0.30137 | 73 | WCM10_Z1-1 | Tissue |
| 10 | 12162822 | G | A | DHTKD1 | p.A899T | 0 | 108 | 0.324324 | 74 | WCM10_Z1-1 | Tissue |
| 10 | 124753459 | C | T | IKZF5 | p.C366Y | 0 | 104 | 0.059406 | 101 | WCM10_Z1-1 | Tissue |
| 10 | 129902969 | G | A | MKI67 | p.R2379* | 0 | 538 | 0.336163 | 589 | WCM10_Z1-1 | Tissue |
| 10 | 129906317 | T | C | MKI67 | p.T1263A | 0 | 418 | 0.356968 | 409 | WCM10_Z1-1 | Tissue |
| 10 | 131640410 | C | T | EBF3 | p.V448M | 0 | 124 | 0.37069 | 116 | WCM10_Z1-1 | Tissue |
| 10 | 17891729 | A | G | MRC1L1 | p.I404V | 0 | 94 | 0.758242 | 91 | WCM10_Z1-1 | Tissue |
| 10 | 23633673 | A | G | C10orf67 | p.Y12H | 0 | 72 | 0.254545 | 55 | WCM10_Z1-1 | Tissue |
| 10 | 30317629 | G | A | KIAA1462 | p.P483L | 0 | 50 | 0.266667 | 60 | WCM10_Z1-1 | Tissue |
| 10 | 44139736 | C | A | ZNF32 | p.G195V | 0 | 376 | 0.319088 | 351 | WCM10_Z1-1 | Tissue |
| 10 | 50740869 | G | A | ERCC6 | p.R48C | 0 | 161 | 0.338129 | 139 | WCM10_Z1-1 | Tissue |
| 10 | 50947784 | A | G | OGDHL | p.F748L | 0 | 86 | 0.313953 | 86 | WCM10_Z1-1 | Tissue |
| 10 | 64957288 | G | A | JMJD1C | p.R1843W | 0 | 101 | 0.1 | 90 | WCM10_Z1-1 | Tissue |
| 10 | 73501496 | C | T | CDH23 | p.R1560C | 0 | 126 | 0.322581 | 124 | WCM10_Z1-1 | Tissue |
| 10 | 73767936 | C | T | CHST3 | p.R383C | 0 | 43 | 0.378378 | 37 | WCM10_Z1-1 | Tissue |
| 10 | 74128105 | C | T | MICU1 | p.E427K | 0 | 74 | 0.37931 | 58 | WCM10_Z1-1 | Tissue |
| 10 | 74810830 | C | T | P4HA1 | p.R294H | 0 | 314 | 0.315412 | 279 | WCM10_Z1-1 | Tissue |
| 10 | 75877841 | C | T | VCL | p.R1107W | 0 | 137 | 0.277228 | 101 | WCM10_Z1-1 | Tissue |
| 10 | 76789389 | G | A | KAT6B | p.V1603I | 0 | 322 | 0.335714 | 280 | WCM10_Z1-1 | Tissue |
| 10 | 76790451 | C | T | KAT6B | p.R1957W | 0 | 206 | 0.328947 | 228 | WCM10_Z1-1 | Tissue |
| 10 | 91514405 | G | A | KIF20B | p.R1493H | 0 | 103 | 0.294118 | 102 | WCM10_Z1-1 | Tissue |
| 10 | 96798695 | C | T | CYP2C8 | p.G417D | 0 | 43 | 0.086957 | 46 | WCM10_Z1-1 | Tissue |
| 1 | 100154787 | C | T | PALMD | p.P324L | 0.005882 | 170 | 0.294118 | 153 | WCM10_Z1-1 | Tissue |
| 1 | 109824326 | G | A | PSRC1 | p.T145M | 0 | 102 | 0.382716 | 81 | WCM10_Z1-1 | Tissue |
| 11 | 118373197 | G | A | KMT2A | p.R2194H | 0 | 305 | 0.328859 | 298 | WCM10_Z1-1 | Tissue |
| 11 | 118498402 | G | A | PHLDB1 | p.R288H | 0 | 144 | 0.380165 | 121 | WCM10_Z1-1 | Tissue |
| 11 | 119051923 | C | T | NLRX1 | p.R771* | 0 | 310 | 0.37037 | 297 | WCM10_Z1-1 | Tissue |
| 11 | 119182104 | G | A | MCAM | p.R479* | 0 | 108 | 0.339806 | 103 | WCM10_Z1-1 | Tissue |
| 11 | 123754810 | G | T | TMEM225 | p.Y145* | 0 | 45 | 0.295455 | 44 | WCM10_Z1-1 | Tissue |
| 11 | 128638164 | G | A | FLI1 | p.A128T | 0.009804 | 102 | 0.378947 | 95 | WCM10_Z1-1 | Tissue |
| 1 | 114524024 | C | T | OLFML3 | p.A285V | 0 | 178 | 0.382199 | 191 | WCM10_Z1-1 | Tissue |
| 1 | 1168367 | G | A | B3GALT6 | p.A237T | 0 | 135 | 0.366667 | 150 | WCM10_Z1-1 | Tissue |
| 1 | 117487574 | G | A | PTGFRN | p.R231H | 0 | 182 | 0.366667 | 180 | WCM10_Z1-1 | Tissue |
| 1 | 120468198 | T | C | NOTCH2 | p.Y1414C | 0 | 138 | 0.380952 | 126 | WCM10_Z1-1 | Tissue |
| 11 | 22833372 | G | A | GAS2 | p.R251H | 0 | 60 | 0.28 | 50 | WCM10_Z1-1 | Tissue |
| 11 | 33163244 | C | T | CSTF3 | p.G65D | 0 | 190 | 0.359281 | 167 | WCM10_Z1-1 | Tissue |
| 11 | 36595188 | C | T | RAG1 | p.R112C | 0 | 458 | 0.341357 | 457 | WCM10_Z1-1 | Tissue |
| 11 | 3712617 | G | A | NUP98 | p.R1472* | 0 | 137 | 0.283333 | 120 | WCM10_Z1-1 | Tissue |
| 11 | 46419100 | G | A | AMBRA1 | p.P1266L | 0 | 115 | 0.326531 | 98 | WCM10_Z1-1 | Tissue |
| 11 | 48146642 | G | C | PTPRJ | p.V333L | 0 | 205 | 0.05814 | 172 | WCM10_Z1-1 | Tissue |
| 11 | 5020909 | C | T | OR51L1 | p.R233C | 0 | 109 | 0.074627 | 134 | WCM10_Z1-1 | Tissue |
| 1 | 150236249 | G | A | CA14 | p.A307T | 0 | 354 | 0.314815 | 324 | WCM10_Z1-1 | Tissue |
| 1 | 150445212 | G | T | RPRD2 | p.S1263I | 0 | 266 | 0.108911 | 202 | WCM10_Z1-1 | Tissue |
| 1 | 150931701 | C | T | SETDB1 | p.T793I | 0 | 110 | 0.286957 | 115 | WCM10_Z1-1 | Tissue |
| 1 | 150938981 | G | A | CERS2 | p.R325C | 0 | 65 | 0.294118 | 51 | WCM10_Z1-1 | Tissue |
| 1 | 150972424 | C | T | FAM63A | p.R251H | 0 | 138 | 0.056452 | 124 | WCM10_Z1-1 | Tissue |
| 1 | 151260814 | C | T | ZNF687 | p.R683W | 0 | 134 | 0.320755 | 159 | WCM10_Z1-1 | Tissue |
| 1 | 152130326 | C | T | RPTN | p.V14I | 0 | 151 | 0.294118 | 119 | WCM10_Z1-1 | Tissue |
| 1 | 152648693 | G | A | LCE2C | p.A68T | 0 | 32 | 0.289474 | 38 | WCM10_Z1-1 | Tissue |
| 1 | 153271634 | A | G | PGLYRP3 | p.F268L | 0 | 141 | 0.262295 | 122 | WCM10_Z1-1 | Tissue |
| 1 | 153752345 | G | A | SLC27A3 | p.R687Q | 0 | 217 | 0.26257 | 179 | WCM10_Z1-1 | Tissue |
| 11 | 5510100 | A | T | OR52D1 | p.D55V | 0 | 557 | 0.295727 | 585 | WCM10_Z1-1 | Tissue |
| 1 | 155295433 | G | A | RUSC1 | p.R595H | 0 | 376 | 0.055851 | 376 | WCM10_Z1-1 | Tissue |
| 11 | 56237501 | G | A | OR5M3 | p.T158I | 0 | 47 | 0.352113 | 71 | WCM10_Z1-1 | Tissue |
| 1 | 156616793 | G | A | BCAN | p.V98I | 0 | 46 | 0.392157 | 51 | WCM10_Z1-1 | Tissue |
| 11 | 59271238 | C | T | OR4D11 | p.R64C | 0 | 655 | 0.282051 | 585 | WCM10_Z1-1 | Tissue |
| 1 | 159284175 | G | A | OR10J3 | p.P92L | 0 | 333 | 0.333333 | 372 | WCM10_Z1-1 | Tissue |
| 11 | 6129180 | G | A | OR56B4 | p.E58K | 0 | 318 | 0.358553 | 304 | WCM10_Z1-1 | Tissue |
| 1 | 16199493 | G | A | SPEN | p.R89Q | 0 | 215 | 0.306122 | 196 | WCM10_Z1-1 | Tissue |
| 11 | 62373382 | G | A | EML3 | p.T576M | 0.003984 | 251 | 0.27451 | 255 | WCM10_Z1-1 | Tissue |
| 11 | 6261787 | G | A | CNGA4 | p.V255I | 0 | 69 | 0.266667 | 60 | WCM10_Z1-1 | Tissue |
| 1 | 162745621 | T | C | DDR2 | p.V679A | 0 | 49 | 0.348837 | 43 | WCM10_Z1-1 | Tissue |
| 11 | 64876348 | G | A | VPS51 | p.A469T | 0 | 41 | 0.4375 | 32 | WCM10_Z1-1 | Tissue |
| 11 | 65375126 | G | A | MAP3K11 | p.R154* | 0 | 298 | 0.358156 | 282 | WCM10_Z1-1 | Tissue |
| 11 | 65819956 | C | T | SF3B2 | p.A34V | 0 | 113 | 0.348624 | 109 | WCM10_Z1-1 | Tissue |
| 11 | 66100748 | G | T | RIN1 | p.P619H | 0 | 82 | 0.256098 | 82 | WCM10_Z1-1 | Tissue |
| 11 | 6622730 | C | T | RRP8 | p.R189Q | 0 | 95 | 0.329545 | 88 | WCM10_Z1-1 | Tissue |
| 11 | 6638002 | C | T | TPP1 | p.R259H | 0 | 237 | 0.328185 | 259 | WCM10_Z1-1 | Tissue |
| 11 | 66483393 | G | C | SPTBN2 | p.R73G | 0 | 34 | 0.097561 | 41 | WCM10_Z1-1 | Tissue |
| 1 | 166890215 | C | T | ILDR2 | p.R538Q | 0 | 110 | 0.310345 | 116 | WCM10_Z1-1 | Tissue |
| 11 | 67059545 | G | A | ANKRD13D | p.E122K | 0 | 73 | 0.324324 | 74 | WCM10_Z1-1 | Tissue |
| 11 | 67223665 | G | A | CABP4 | p.E125K | 0 | 114 | 0.413043 | 92 | WCM10_Z1-1 | Tissue |
| 11 | 67379443 | C | T | NDUFV1 | p.R386C | 0 | 68 | 0.084507 | 71 | WCM10_Z1-1 | Tissue |
| 11 | 67379621 | G | A | NDUFV1 | p.R398H | 0 | 48 | 0.276596 | 47 | WCM10_Z1-1 | Tissue |
| 1 | 168073820 | C | T | GPR161 | p.R90H | 0 | 128 | 0.330357 | 112 | WCM10_Z1-1 | Tissue |
| 11 | 68571499 | G | A | CPT1A | p.P175L | 0 | 183 | 0.358025 | 162 | WCM10_Z1-1 | Tissue |
| 11 | 69462905 | G | A | CCND1 | p.D240N | 0 | 170 | 0.421769 | 147 | WCM10_Z1-1 | Tissue |
| 11 | 70052301 | C | T | FADD | p.R117C | 0 | 124 | 0.354839 | 124 | WCM10_Z1-1 | Tissue |
| 11 | 70253434 | T | C | CTTN | p.S11P | 0 | 41 | 0.085106 | 47 | WCM10_Z1-1 | Tissue |
| 11 | 70281224 | C | T | CTTN | p.R537W | 0 | 106 | 0.111111 | 72 | WCM10_Z1-1 | Tissue |
| 11 | 71723452 | T | C | NUMA1 | p.K1549E | 0 | 94 | 0.287356 | 87 | WCM10_Z1-1 | Tissue |
| 11 | 71729532 | C | T | NUMA1 | p.R257H | 0 | 87 | 0.4 | 75 | WCM10_Z1-1 | Tissue |
| 1 | 171765760 | A | G | METTL13 | p.H655R | 0 | 87 | 0.382022 | 89 | WCM10_Z1-1 | Tissue |
| 11 | 72600915 | G | A | FCHSD2 | p.R334C | 0 | 98 | 0.314286 | 70 | WCM10_Z1-1 | Tissue |
| 1 | 173910473 | T | C | RC3H1 | p.K1061E | 0 | 95 | 0.352113 | 71 | WCM10_Z1-1 | Tissue |
| 1 | 175046703 | T | C | TNN | p.V50A | 0 | 164 | 0.346939 | 147 | WCM10_Z1-1 | Tissue |
| 11 | 75188686 | C | T | GDPD5 | p.R32H | 0 | 46 | 0.388889 | 54 | WCM10_Z1-1 | Tissue |
| 1 | 17607185 | G | A | PADI3 | p.R552H | 0 | 152 | 0.07483 | 147 | WCM10_Z1-1 | Tissue |
| 1 | 176738812 | G | A | PAPPA2 | p.G1465S | 0 | 70 | 0.111111 | 72 | WCM10_Z1-1 | Tissue |
| 11 | 77991936 | T | G | GAB2 | p.K29N | 0 | 92 | 0.057143 | 105 | WCM10_Z1-1 | Tissue |
| 1 | 180914662 | G | A | KIAA1614 | p.G1171S | 0 | 101 | 0.359551 | 89 | WCM10_Z1-1 | Tissue |
| 1 | 19166643 | C | T | TAS1R2 | p.C657Y | 0 | 120 | 0.395161 | 124 | WCM10_Z1-1 | Tissue |
| 11 | 9517890 | G | A | ZNF143 | p.C269Y | 0 | 118 | 0.309735 | 113 | WCM10_Z1-1 | Tissue |
| 1 | 196927096 | C | T | CFHR2 | p.A169V | 0 | 47 | 0.44898 | 49 | WCM10_Z1-1 | Tissue |
| 1 | 202457713 | C | T | PPP1R12B | p.R635C | 0 | 55 | 0.240741 | 54 | WCM10_Z1-1 | Tissue |
| 1 | 203678603 | G | A | ATP2B4 | p.V578I | 0 | 203 | 0.316062 | 193 | WCM10_Z1-1 | Tissue |
| 1 | 204438809 | C | T | PIK3C2B | p.R41Q | 0 | 100 | 0.345238 | 84 | WCM10_Z1-1 | Tissue |
| 1 | 207288767 | G | A | C4BPA | p.R112Q | 0 | 100 | 0.271739 | 92 | WCM10_Z1-1 | Tissue |
| 12 | 100453127 | A | G | UHRF1BP1L | p.F643S | 0 | 98 | 0.054054 | 111 | WCM10_Z1-1 | Tissue |
| 12 | 104086654 | A | G | STAB2 | p.H1121R | 0 | 114 | 0.264957 | 117 | WCM10_Z1-1 | Tissue |
| 12 | 108104257 | C | T | PWP1 | p.R456W | 0 | 127 | 0.336538 | 104 | WCM10_Z1-1 | Tissue |
| 12 | 111856367 | C | T | SH2B3 | p.R140C | 0 | 33 | 0.272727 | 33 | WCM10_Z1-1 | Tissue |
| 12 | 117158157 | T | C | C12orf49 | p.N122D | 0 | 165 | 0.317568 | 148 | WCM10_Z1-1 | Tissue |
| 12 | 118517168 | C | T | VSIG10 | p.S303N | 0 | 173 | 0.247788 | 113 | WCM10_Z1-1 | Tissue |
| 12 | 121880281 | G | A | KDM2B | p.P988L | 0 | 187 | 0.385621 | 153 | WCM10_Z1-1 | Tissue |
| 12 | 122091153 | C | T | MORN3 | p.R159H | 0 | 234 | 0.055866 | 179 | WCM10_Z1-1 | Tissue |
| 12 | 122839066 | C | T | CLIP1 | p.R414Q | 0 | 37 | 0.09375 | 32 | WCM10_Z1-1 | Tissue |
| 12 | 124497247 | C | A | ZNF664 | p.L186I | 0 | 196 | 0.0625 | 176 | WCM10_Z1-1 | Tissue |
| 12 | 132210002 | G | A | SFSWAP | p.C220Y | 0 | 79 | 0.066667 | 60 | WCM10_Z1-1 | Tissue |
| 12 | 132400519 | G | A | ULK1 | p.V565I | 0 | 37 | 0.2 | 40 | WCM10_Z1-1 | Tissue |
| 12 | 19436421 | G | A | PLEKHA5 | p.W501* | 0 | 182 | 0.308036 | 224 | WCM10_Z1-1 | Tissue |
| 1 | 223396618 | T | C | SUSD4 | p.T473A | 0 | 59 | 0.311111 | 45 | WCM10_Z1-1 | Tissue |
| 1 | 22446767 | C | T | WNT4 | p.D278N | 0 | 148 | 0.316547 | 139 | WCM10_Z1-1 | Tissue |
| 1 | 226110032 | C | T | PYCR2 | p.D64N | 0 | 80 | 0.371134 | 97 | WCM10_Z1-1 | Tissue |
| 12 | 2702423 | C | T | CACNA1C | p.P859S | 0 | 49 | 0.105263 | 38 | WCM10_Z1-1 | Tissue |
| 1 | 228602514 | G | A | TRIM17 | p.A87V | 0 | 104 | 0.436782 | 87 | WCM10_Z1-1 | Tissue |
| 1 | 229635471 | G | A | NUP133 | p.S203L | 0 | 72 | 0.403846 | 52 | WCM10_Z1-1 | Tissue |
| 12 | 2997144 | C | T | RHNO1 | p.A79V | 0 | 185 | 0.300613 | 163 | WCM10_Z1-1 | Tissue |
| 1 | 231048444 | A | T | TTC13 | p.Y718* | 0 | 161 | 0.101523 | 197 | WCM10_Z1-1 | Tissue |
| 1 | 23768689 | C | T | ASAP3 | p.A221T | 0 | 53 | 0.355932 | 59 | WCM10_Z1-1 | Tissue |
| 1 | 241953906 | A | T | WDR64 | p.S959C | 0 | 289 | 0.314879 | 289 | WCM10_Z1-1 | Tissue |
| 12 | 41966404 | C | T | PDZRN4 | p.S608F | 0 | 119 | 0.412214 | 131 | WCM10_Z1-1 | Tissue |
| 1 | 24201179 | G | T | CNR2 | p.A310D | 0 | 81 | 0.074074 | 81 | WCM10_Z1-1 | Tissue |
| 1 | 242271008 | T | G | PLD5 | p.I402L | 0 | 53 | 0.297872 | 47 | WCM10_Z1-1 | Tissue |
| 1 | 2428326 | C | T | PLCH2 | p.Q665* | 0 | 119 | 0.12069 | 116 | WCM10_Z1-1 | Tissue |
| 1 | 24406580 | C | T | MYOM3 | p.V838I | 0 | 66 | 0.293103 | 58 | WCM10_Z1-1 | Tissue |
| 1 | 24663153 | G | A | GRHL3 | p.A150T | 0 | 271 | 0.337165 | 261 | WCM10_Z1-1 | Tissue |
| 12 | 48095371 | C | T | RPAP3 | p.R57Q | 0 | 43 | 0.320755 | 53 | WCM10_Z1-1 | Tissue |
| 12 | 48258907 | C | T | VDR | p.R67H | 0 | 91 | 0.25 | 76 | WCM10_Z1-1 | Tissue |
| 12 | 49259520 | C | T | RND1 | p.V11M | 0 | 115 | 0.407767 | 103 | WCM10_Z1-1 | Tissue |
| 12 | 49427360 | C | T | KMT2D | p.G3710R | 0 | 76 | 0.305556 | 72 | WCM10_Z1-1 | Tissue |
| 12 | 49431633 | C | T | KMT2D | p.G3169D | 0 | 74 | 0.321429 | 84 | WCM10_Z1-1 | Tissue |
| 12 | 51019819 | C | T | DIP2B | p.P54L | 0 | 83 | 0.274194 | 62 | WCM10_Z1-1 | Tissue |
| 12 | 53170972 | C | T | KRT76 | p.R35H | 0.006289 | 159 | 0.09396 | 149 | WCM10_Z1-1 | Tissue |
| 12 | 53215804 | C | T | KRT79 | p.G487D | 0 | 111 | 0.373494 | 83 | WCM10_Z1-1 | Tissue |
| 12 | 53467149 | C | T | SPRYD3 | p.R205H | 0 | 133 | 0.354167 | 96 | WCM10_Z1-1 | Tissue |
| 12 | 53691689 | C | T | PFDN5 | p.T88I | 0 | 104 | 0.35 | 100 | WCM10_Z1-1 | Tissue |
| 12 | 56420735 | C | T | IKZF4 | p.R153W | 0 | 84 | 0.317647 | 85 | WCM10_Z1-1 | Tissue |
| 12 | 57000084 | G | A | BAZ2A | p.R738W | 0 | 198 | 0.281609 | 174 | WCM10_Z1-1 | Tissue |
| 12 | 57968961 | G | A | KIF5A | p.R604Q | 0 | 158 | 0.41791 | 134 | WCM10_Z1-1 | Tissue |
| 1 | 26156119 | G | A | MTFR1L | p.E191K | 0 | 286 | 0.310219 | 274 | WCM10_Z1-1 | Tissue |
| 12 | 66742840 | C | T | GRIP1 | p.G1064S | 0 | 215 | 0.331897 | 232 | WCM10_Z1-1 | Tissue |
| 12 | 6760375 | G | A | ING4 | p.R246W | 0 | 101 | 0.415929 | 113 | WCM10_Z1-1 | Tissue |
| 12 | 6837155 | G | A | COPS7A | p.A76T | 0 | 213 | 0.270833 | 192 | WCM10_Z1-1 | Tissue |
| 12 | 71533538 | G | T | TSPAN8 | p.L72M | 0 | 159 | 0.331081 | 148 | WCM10_Z1-1 | Tissue |
| 12 | 7289685 | G | A | CLSTN3 | p.V397I | 0 | 142 | 0.256 | 125 | WCM10_Z1-1 | Tissue |
| 12 | 80771656 | G | T | OTOGL | p.C2276F | 0 | 162 | 0.441667 | 120 | WCM10_Z1-1 | Tissue |
| 1 | 28477310 | C | T | PTAFR | p.V75I | 0 | 77 | 0.333333 | 51 | WCM10_Z1-1 | Tissue |
| 12 | 9075272 | G | A | PHC1 | p.R167Q | 0 | 76 | 0.3125 | 64 | WCM10_Z1-1 | Tissue |
| 1 | 29611288 | C | T | PTPRU | p.S742L | 0 | 72 | 0.304348 | 69 | WCM10_Z1-1 | Tissue |
| 1 | 29631359 | C | T | PTPRU | p.A924V | 0 | 45 | 0.342857 | 35 | WCM10_Z1-1 | Tissue |
| 13 | 110435972 | T | C | IRS2 | p.Y810C | 0 | 59 | 0.373333 | 75 | WCM10_Z1-1 | Tissue |
| 13 | 110804766 | C | T | COL4A1 | p.E1615K | 0 | 62 | 0.230769 | 52 | WCM10_Z1-1 | Tissue |
| 13 | 110817289 | C | T | COL4A1 | p.G1357E | 0 | 113 | 0.340909 | 88 | WCM10_Z1-1 | Tissue |
| 13 | 114525098 | C | T | GAS6 | p.G572D | 0 | 43 | 0.073171 | 41 | WCM10_Z1-1 | Tissue |
| 1 | 31206686 | A | G | LAPTM5 | p.S260P | 0 | 57 | 0.346939 | 49 | WCM10_Z1-1 | Tissue |
| 1 | 32052539 | G | A | TINAGL1 | p.V455I | 0 | 167 | 0.351351 | 148 | WCM10_Z1-1 | Tissue |
| 13 | 25276082 | G | A | ATP12A | p.V631I | 0 | 70 | 0.449438 | 89 | WCM10_Z1-1 | Tissue |
| 13 | 25840368 | G | A | MTMR6 | p.R121* | 0 | 127 | 0.302817 | 142 | WCM10_Z1-1 | Tissue |
| 13 | 29041690 | C | T | FLT1 | p.M43I | 0 | 48 | 0.314286 | 35 | WCM10_Z1-1 | Tissue |
| 1 | 34209090 | C | T | CSMD2 | p.R655H | 0 | 109 | 0.055556 | 126 | WCM10_Z1-1 | Tissue |
| 13 | 47303015 | G | A | LRCH1 | p.E600K | 0 | 183 | 0.353535 | 198 | WCM10_Z1-1 | Tissue |
| 1 | 35260209 | G | A | GJA4 | p.R132H | 0 | 119 | 0.319672 | 122 | WCM10_Z1-1 | Tissue |
| 1 | 36642123 | C | T | MAP7D1 | p.R392C | 0 | 40 | 0.151515 | 33 | WCM10_Z1-1 | Tissue |
| 1 | 36859493 | G | A | LSM10 | p.R80C | 0 | 145 | 0.325581 | 129 | WCM10_Z1-1 | Tissue |
| 13 | 73345226 | C | T | DIS3 | p.V555M | 0 | 125 | 0.484848 | 66 | WCM10_Z1-1 | Tissue |
| 13 | 75936218 | C | T | TBC1D4 | p.A342T | 0 | 56 | 0.114286 | 35 | WCM10_Z1-1 | Tissue |
| 13 | 96205173 | G | A | CLDN10 | p.D56N | 0 | 254 | 0.317181 | 227 | WCM10_Z1-1 | Tissue |
| 14 | 100126037 | G | A | HHIPL1 | p.R440H | 0 | 44 | 0.302326 | 43 | WCM10_Z1-1 | Tissue |
| 14 | 102500356 | G | A | DYNC1H1 | p.R3486H | 0 | 146 | 0.313043 | 115 | WCM10_Z1-1 | Tissue |
| 14 | 105395129 | G | A | PLD4 | p.A110T | 0 | 65 | 0.058824 | 68 | WCM10_Z1-1 | Tissue |
| 1 | 41486065 | G | A | SLFNL1 | p.R90W | 0 | 155 | 0.351351 | 148 | WCM10_Z1-1 | Tissue |
| 14 | 20216469 | A | G | OR4Q3 | p.M295V | 0 | 100 | 0.216495 | 97 | WCM10_Z1-1 | Tissue |
| 14 | 20482854 | A | T | OR4K14 | p.L167M | 0 | 340 | 0.328042 | 378 | WCM10_Z1-1 | Tissue |
| 14 | 21822692 | C | T | SUPT16H | p.D890N | 0 | 159 | 0.374233 | 163 | WCM10_Z1-1 | Tissue |
| 14 | 23345382 | G | A | LRP10 | p.E409K | 0 | 142 | 0.344371 | 151 | WCM10_Z1-1 | Tissue |
| 1 | 43205707 | C | A | CLDN19 | p.G10C | 0 | 39 | 0.06383 | 47 | WCM10_Z1-1 | Tissue |
| 14 | 51411117 | G | A | PYGL | p.A2V | 0 | 175 | 0.368715 | 179 | WCM10_Z1-1 | Tissue |
| 14 | 53184828 | G | A | PSMC6 | p.V187I | 0 | 31 | 0.421053 | 38 | WCM10_Z1-1 | Tissue |
| 14 | 64519872 | C | T | SYNE2 | p.R3081W | 0 | 104 | 0.224719 | 89 | WCM10_Z1-1 | Tissue |
| 14 | 65268517 | C | T | SPTB | p.S201N | 0 | 46 | 0.403509 | 57 | WCM10_Z1-1 | Tissue |
| 14 | 74205720 | G | A | ELMSAN1 | p.P331L | 0 | 99 | 0.336957 | 92 | WCM10_Z1-1 | Tissue |
| 14 | 79454417 | G | A | NRXN3 | p.M692I | 0 | 69 | 0.341772 | 79 | WCM10_Z1-1 | Tissue |
| 14 | 88652390 | G | A | KCNK10 | p.A369V | 0 | 40 | 0.311111 | 45 | WCM10_Z1-1 | Tissue |
| 14 | 95597865 | T | C | DICER1 | p.Q140R | 0 | 95 | 0.052632 | 95 | WCM10_Z1-1 | Tissue |
| 15 | 100594173 | G | A | ADAMTS17 | p.R742C | 0 | 410 | 0.318318 | 333 | WCM10_Z1-1 | Tissue |
| 15 | 100802621 | T | C | ADAMTS17 | p.H270R | 0 | 92 | 0.218182 | 110 | WCM10_Z1-1 | Tissue |
| 15 | 23048918 | C | T | NIPA1 | p.V301I | 0 | 313 | 0.325792 | 221 | WCM10_Z1-1 | Tissue |
| 15 | 31339412 | C | T | TRPM1 | p.G556R | 0 | 152 | 0.362903 | 124 | WCM10_Z1-1 | Tissue |
| 15 | 42454660 | C | T | VPS39 | p.R743Q | 0 | 106 | 0.292035 | 113 | WCM10_Z1-1 | Tissue |
| 15 | 45408397 | G | A | DUOXA2 | p.R94H | 0 | 191 | 0.051546 | 194 | WCM10_Z1-1 | Tissue |
| 15 | 50190412 | G | T | ATP8B4 | p.L776I | 0 | 78 | 0.371429 | 70 | WCM10_Z1-1 | Tissue |
| 15 | 55527089 | A | G | RAB27A | p.L15S | 0 | 115 | 0.313726 | 102 | WCM10_Z1-1 | Tissue |
| 15 | 56735889 | G | A | MNS1 | p.R284W | 0 | 147 | 0.292308 | 130 | WCM10_Z1-1 | Tissue |
| 15 | 59179239 | C | T | SLTM | p.A924T | 0 | 51 | 0.390244 | 41 | WCM10_Z1-1 | Tissue |
| 15 | 62209823 | A | G | VPS13C | p.F2591S | 0 | 48 | 0.071429 | 56 | WCM10_Z1-1 | Tissue |
| 15 | 63014597 | G | A | TLN2 | p.V1013M | 0 | 36 | 0.268293 | 41 | WCM10_Z1-1 | Tissue |
| 15 | 66727483 | G | A | MAP2K1 | p.D67N | 0 | 88 | 0.303571 | 56 | WCM10_Z1-1 | Tissue |
| 15 | 72292209 | G | A | MYO9A | p.P485S | 0 | 127 | 0.057143 | 105 | WCM10_Z1-1 | Tissue |
| 15 | 72581515 | C | T | CELF6 | p.D332N | 0 | 38 | 0.105263 | 38 | WCM10_Z1-1 | Tissue |
| 15 | 74043452 | G | A | C15orf59 | p.P7L | 0 | 197 | 0.167702 | 161 | WCM10_Z1-1 | Tissue |
| 15 | 74336927 | G | A | PML | p.A743T | 0 | 219 | 0.35567 | 194 | WCM10_Z1-1 | Tissue |
| 15 | 74426576 | C | T | ISLR2 | p.A494V | 0 | 118 | 0.333333 | 81 | WCM10_Z1-1 | Tissue |
| 15 | 75704008 | G | A | SIN3A | p.P278L | 0 | 130 | 0.360465 | 86 | WCM10_Z1-1 | Tissue |
| 15 | 81592486 | C | T | IL16 | p.P940L | 0 | 108 | 0.37069 | 116 | WCM10_Z1-1 | Tissue |
| 15 | 91009567 | C | T | IQGAP1 | p.A645V | 0 | 99 | 0.313253 | 83 | WCM10_Z1-1 | Tissue |
| 15 | 91490037 | G | A | UNC45A | p.A465T | 0 | 131 | 0.25 | 124 | WCM10_Z1-1 | Tissue |
| 16 | 11145464 | G | A | CLEC16A | p.R654Q | 0 | 80 | 0.304348 | 92 | WCM10_Z1-1 | Tissue |
| 16 | 1252389 | G | A | CACNA1H | p.G647S | 0 | 101 | 0.3875 | 80 | WCM10_Z1-1 | Tissue |
| 16 | 17202563 | G | A | XYLT1 | p.R957W | 0 | 185 | 0.284024 | 169 | WCM10_Z1-1 | Tissue |
| 16 | 2107141 | C | G | TSC2 | p.H270Q | 0 | 67 | 0.307692 | 78 | WCM10_Z1-1 | Tissue |
| 16 | 21145739 | C | T | DNAH3 | p.R308Q | 0 | 123 | 0.228916 | 83 | WCM10_Z1-1 | Tissue |
| 16 | 2347906 | C | T | ABCA3 | p.R638H | 0 | 57 | 0.380952 | 42 | WCM10_Z1-1 | Tissue |
| 16 | 23646726 | G | T | PALB2 | p.L381I | 0 | 168 | 0.301887 | 159 | WCM10_Z1-1 | Tissue |
| 16 | 28841229 | G | A | ATXN2L | p.R295H | 0 | 67 | 0.404494 | 89 | WCM10_Z1-1 | Tissue |
| 16 | 29912717 | C | T | ASPHD1 | p.T142M | 0 | 128 | 0.291262 | 103 | WCM10_Z1-1 | Tissue |
| 16 | 30536199 | C | T | ZNF768 | p.R421H | 0 | 74 | 0.276316 | 76 | WCM10_Z1-1 | Tissue |
| 16 | 31235763 | C | T | TRIM72 | p.A374V | 0 | 47 | 0.354839 | 31 | WCM10_Z1-1 | Tissue |
| 16 | 3169401 | C | T | ZNF205 | p.P247L | 0 | 37 | 0.358974 | 39 | WCM10_Z1-1 | Tissue |
| 16 | 4016348 | G | A | ADCY9 | p.R1164C | 0 | 244 | 0.364078 | 206 | WCM10_Z1-1 | Tissue |
| 16 | 4562820 | C | T | CDIP1 | p.V163M | 0 | 131 | 0.173469 | 98 | WCM10_Z1-1 | Tissue |
| 16 | 47143528 | T | C | NETO2 | p.N250S | 0 | 134 | 0.377193 | 114 | WCM10_Z1-1 | Tissue |
| 16 | 4827917 | G | T | 12-Sep | p.L320M | 0 | 110 | 0.13 | 100 | WCM10_Z1-1 | Tissue |
| 16 | 5046920 | G | A | SEC14L5 | p.R282Q | 0 | 129 | 0.330275 | 109 | WCM10_Z1-1 | Tissue |
| 1 | 6530820 | C | T | PLEKHG5 | p.R562H | 0 | 119 | 0.320896 | 134 | WCM10_Z1-1 | Tissue |
| 16 | 53907736 | G | A | FTO | p.G312S | 0 | 116 | 0.052632 | 95 | WCM10_Z1-1 | Tissue |
| 16 | 57059640 | G | A | NLRC5 | p.R262H | 0 | 340 | 0.062802 | 207 | WCM10_Z1-1 | Tissue |
| 16 | 57059850 | G | A | NLRC5 | p.C332Y | 0 | 238 | 0.134752 | 141 | WCM10_Z1-1 | Tissue |
| 16 | 57295954 | A | C | PLLP | p.I55S | 0 | 89 | 0.578947 | 38 | WCM10_Z1-1 | Tissue |
| 16 | 66613674 | C | T | CMIM2 | p.A55V | 0 | 156 | 0.372881 | 118 | WCM10_Z1-1 | Tissue |
| 16 | 67263009 | G | A | TMEM208 | p.R172Q | 0 | 32 | 0.114286 | 35 | WCM10_Z1-1 | Tissue |
| 16 | 67293534 | C | A | SLC9A5 | p.S562Y | 0 | 276 | 0.056034 | 232 | WCM10_Z1-1 | Tissue |
| 16 | 683405 | G | A | WFIKKN1 | p.R332H | 0 | 121 | 0.412214 | 131 | WCM10_Z1-1 | Tissue |
| 16 | 69201044 | C | T | CIRH1A | p.R634W | 0 | 178 | 0.315789 | 152 | WCM10_Z1-1 | Tissue |
| 16 | 70505140 | C | T | FUK | p.S409F | 0 | 68 | 0.362069 | 58 | WCM10_Z1-1 | Tissue |
| 16 | 70731097 | A | G | VAC14 | p.S634P | 0 | 125 | 0.329787 | 94 | WCM10_Z1-1 | Tissue |
| 16 | 72135085 | G | A | DHX38 | p.R460H | 0 | 128 | 0.063492 | 126 | WCM10_Z1-1 | Tissue |
| 16 | 88706470 | G | A | IL17C | p.R195H | 0 | 42 | 0.333333 | 51 | WCM10_Z1-1 | Tissue |
| 16 | 89346600 | G | A | ANKRD11 | p.P2117L | 0 | 54 | 0.409091 | 44 | WCM10_Z1-1 | Tissue |
| 16 | 89922615 | C | T | SPIRE2 | p.P358L | 0 | 32 | 0.388889 | 36 | WCM10_Z1-1 | Tissue |
| 17 | 1028646 | G | C | ABR | p.P40A | 0 | 479 | 0.336043 | 369 | WCM10_Z1-1 | Tissue |
| 17 | 15942818 | C | T | NCOR1 | p.G2295D | 0 | 106 | 0.07767 | 103 | WCM10_Z1-1 | Tissue |
| 17 | 1637421 | G | A | WDR81 | p.R1697H | 0 | 36 | 0.372549 | 51 | WCM10_Z1-1 | Tissue |
| 17 | 18024036 | C | T | MYO15A | p.A641V | 0 | 61 | 0.410256 | 39 | WCM10_Z1-1 | Tissue |
| 17 | 21319088 | G | A | KCNJ12 | p.G145D | 0 | 457 | 0.116592 | 446 | WCM10_Z1-1 | Tissue |
| 17 | 27186078 | C | T | ERAL1 | p.R353* | 0 | 297 | 0.293286 | 283 | WCM10_Z1-1 | Tissue |
| 17 | 27207745 | C | T | FLOT2 | p.V412M | 0 | 82 | 0.263889 | 72 | WCM10_Z1-1 | Tissue |
| 17 | 27899641 | G | A | TP53I13 | p.R332Q | 0 | 84 | 0.369048 | 84 | WCM10_Z1-1 | Tissue |
| 17 | 27899662 | G | A | TP53I13 | p.R339H | 0 | 74 | 0.231707 | 82 | WCM10_Z1-1 | Tissue |
| 17 | 28817222 | C | T | GOSR1 | p.R143* | 0 | 37 | 0.290323 | 31 | WCM10_Z1-1 | Tissue |
| 17 | 31260256 | G | A | TMEM98 | p.D66N | 0 | 225 | 0.293785 | 177 | WCM10_Z1-1 | Tissue |
| 17 | 3526742 | C | T | SHPK | p.D180N | 0 | 142 | 0.352381 | 105 | WCM10_Z1-1 | Tissue |
| 17 | 37226179 | G | A | PLXDC1 | p.A438V | 0 | 105 | 0.057971 | 69 | WCM10_Z1-1 | Tissue |
| 17 | 37342815 | C | T | CACNB1 | p.D188N | 0 | 108 | 0.367925 | 106 | WCM10_Z1-1 | Tissue |
| 17 | 3844528 | G | A | ATP2A3 | p.R613C | 0 | 223 | 0.338798 | 183 | WCM10_Z1-1 | Tissue |
| 17 | 39324367 | C | T | KRTAP4-3 | p.G20S | 0 | 42 | 0.410256 | 39 | WCM10_Z1-1 | Tissue |
| 17 | 39644614 | C | T | KRT36 | p.A194T | 0 | 100 | 0.360465 | 86 | WCM10_Z1-1 | Tissue |
| 17 | 40028079 | G | A | ACLY | p.R934W | 0 | 123 | 0.096386 | 83 | WCM10_Z1-1 | Tissue |
| 17 | 40474338 | C | T | STAT3 | p.R688Q | 0 | 75 | 0.337662 | 77 | WCM10_Z1-1 | Tissue |
| 17 | 40705267 | G | A | HSD17B1 | p.A75T | 0 | 342 | 0.381924 | 343 | WCM10_Z1-1 | Tissue |
| 17 | 40717048 | C | T | COASY | p.R429W | 0 | 212 | 0.351598 | 219 | WCM10_Z1-1 | Tissue |
| 17 | 41907051 | C | T | MPP3 | p.V138I | 0 | 127 | 0.313559 | 118 | WCM10_Z1-1 | Tissue |
| 17 | 42018863 | G | A | PPY | p.R54C | 0 | 77 | 0.380282 | 71 | WCM10_Z1-1 | Tissue |
| 17 | 42635388 | G | A | FZD2 | p.R111H | 0 | 160 | 0.355263 | 152 | WCM10_Z1-1 | Tissue |
| 17 | 4701403 | C | T | PSMB6 | p.R178W | 0 | 188 | 0.314721 | 197 | WCM10_Z1-1 | Tissue |
| 17 | 48070847 | G | A | DLX3 | p.Q145* | 0 | 56 | 0.333333 | 51 | WCM10_Z1-1 | Tissue |
| 17 | 48273721 | C | T | COL1A1 | p.R304H | 0 | 177 | 0.305556 | 144 | WCM10_Z1-1 | Tissue |
| 17 | 48456169 | C | T | EME1 | p.T342M | 0 | 59 | 0.309524 | 42 | WCM10_Z1-1 | Tissue |
| 17 | 48462601 | C | T | LRRC59 | p.R185Q | 0 | 392 | 0.34005 | 397 | WCM10_Z1-1 | Tissue |
| 17 | 48538075 | G | A | ACSF2 | p.G56R | 0 | 138 | 0.388889 | 108 | WCM10_Z1-1 | Tissue |
| 17 | 526892 | G | A | VPS53 | p.R333C | 0 | 115 | 0.347826 | 115 | WCM10_Z1-1 | Tissue |
| 17 | 53345166 | C | T | HLF | p.P57L | 0 | 139 | 0.067227 | 119 | WCM10_Z1-1 | Tissue |
| 17 | 56271132 | G | A | EPX | p.R135Q | 0 | 121 | 0.326733 | 101 | WCM10_Z1-1 | Tissue |
| 17 | 56382463 | C | T | BZRAP1 | p.G1835S | 0 | 187 | 0.068571 | 175 | WCM10_Z1-1 | Tissue |
| 17 | 58233977 | G | A | CA4 | p.V57I | 0 | 115 | 0.359223 | 103 | WCM10_Z1-1 | Tissue |
| 17 | 58952033 | G | A | BCAS3 | p.V199I | 0 | 91 | 0.326531 | 98 | WCM10_Z1-1 | Tissue |
| 17 | 58979988 | G | A | BCAS3 | p.R249Q | 0 | 102 | 0.358025 | 81 | WCM10_Z1-1 | Tissue |
| 17 | 61958873 | C | T | GH2 | p.R6Q | 0 | 40 | 0.245283 | 53 | WCM10_Z1-1 | Tissue |
| 17 | 66339822 | G | A | ARSG | p.R99H | 0 | 33 | 0.078947 | 38 | WCM10_Z1-1 | Tissue |
| 17 | 725651 | C | T | NXN | p.R220Q | 0 | 72 | 0.296875 | 64 | WCM10_Z1-1 | Tissue |
| 17 | 72576202 | G | A | CD300LD | p.P175L | 0 | 80 | 0.320513 | 78 | WCM10_Z1-1 | Tissue |
| 17 | 73317762 | C | T | GRB2 | p.R149Q | 0 | 41 | 0.268293 | 41 | WCM10_Z1-1 | Tissue |
| 17 | 73910092 | G | A | FBF1 | p.R996C | 0 | 159 | 0.421875 | 128 | WCM10_Z1-1 | Tissue |
| 17 | 76130568 | G | A | TMC8 | p.V304I | 0 | 57 | 0.06 | 50 | WCM10_Z1-1 | Tissue |
| 17 | 76446422 | G | A | DNAH17 | p.R3647C | 0 | 155 | 0.103448 | 116 | WCM10_Z1-1 | Tissue |
| 17 | 76455208 | T | A | DNAH17 | p.I3241F | 0 | 161 | 0.289062 | 128 | WCM10_Z1-1 | Tissue |
| 17 | 7660519 | G | A | DNAH2 | p.R672H | 0 | 89 | 0.080808 | 99 | WCM10_Z1-1 | Tissue |
| 17 | 78972949 | C | T | CHMP6 | p.S201L | 0 | 35 | 0.371429 | 35 | WCM10_Z1-1 | Tissue |
| 17 | 7907445 | G | A | GUCY2D | p.E333K | 0 | 154 | 0.337662 | 154 | WCM10_Z1-1 | Tissue |
| 17 | 79567372 | G | A | NPLOC4 | p.R306W | 0 | 164 | 0.076389 | 144 | WCM10_Z1-1 | Tissue |
| 17 | 80373449 | C | T | OGFOD3 | p.W43* | 0 | 111 | 0.325843 | 89 | WCM10_Z1-1 | Tissue |
| 17 | 80436783 | G | A | NARF | p.A210T | 0 | 58 | 0.487805 | 41 | WCM10_Z1-1 | Tissue |
| 17 | 8053361 | G | A | PER1 | p.R153C | 0 | 198 | 0.360465 | 172 | WCM10_Z1-1 | Tissue |
| 17 | 80616552 | C | T | RAB40B | p.R127H | 0 | 78 | 0.328125 | 64 | WCM10_Z1-1 | Tissue |
| 17 | 8219452 | C | T | ARHGEF15 | p.R564C | 0 | 142 | 0.443299 | 97 | WCM10_Z1-1 | Tissue |
| 1 | 79094070 | G | A | IFI44L | p.R157Q | 0 | 70 | 0.208791 | 91 | WCM10_Z1-1 | Tissue |
| 18 | 10487899 | C | T | APCDD1 | p.S470L | 0 | 136 | 0.273585 | 106 | WCM10_Z1-1 | Tissue |
| 18 | 28991355 | C | T | DSG4 | p.R767W | 0 | 141 | 0.39759 | 83 | WCM10_Z1-1 | Tissue |
| 18 | 42533188 | G | A | SETBP1 | p.V1295M | 0 | 62 | 0.276923 | 65 | WCM10_Z1-1 | Tissue |
| 1 | 8425976 | C | T | RERE | p.R448Q | 0 | 57 | 0.295455 | 44 | WCM10_Z1-1 | Tissue |
| 1 | 84791363 | G | A | SAMD13 | p.V53M | 0 | 91 | 0.347222 | 72 | WCM10_Z1-1 | Tissue |
| 18 | 60527681 | G | A | PHLPP1 | p.R638H | 0.003401 | 294 | 0.356667 | 300 | WCM10_Z1-1 | Tissue |
| 18 | 6239790 | C | T | L3MBTL4 | p.A212T | 0 | 499 | 0.275862 | 493 | WCM10_Z1-1 | Tissue |
| 1 | 86957043 | C | T | CLCA1 | p.R485C | 0 | 77 | 0.067568 | 74 | WCM10_Z1-1 | Tissue |
| 18 | 72998519 | C | T | TSHZ1 | p.A386V | 0 | 217 | 0.31982 | 222 | WCM10_Z1-1 | Tissue |
| 18 | 74154850 | C | T | ZNF516 | p.R54H | 0 | 158 | 0.084507 | 142 | WCM10_Z1-1 | Tissue |
| 1 | 874456 | G | A | SAMD11 | p.R156H | 0 | 93 | 0.340206 | 97 | WCM10_Z1-1 | Tissue |
| 1 | 87538659 | G | A | HS2ST1 | p.R56Q | 0 | 31 | 0.090909 | 44 | WCM10_Z1-1 | Tissue |
| 1 | 898494 | G | A | KLHL17 | p.G350R | 0 | 126 | 0.346457 | 127 | WCM10_Z1-1 | Tissue |
| 1 | 900543 | C | T | KLHL17 | p.T634M | 0 | 146 | 0.323308 | 133 | WCM10_Z1-1 | Tissue |
| 1 | 908671 | G | A | PLEKHN1 | p.G472S | 0 | 117 | 0.313726 | 102 | WCM10_Z1-1 | Tissue |
| 19 | 10818202 | T | C | QTRT1 | p.I186T | 0 | 151 | 0.372263 | 137 | WCM10_Z1-1 | Tissue |
| 19 | 1127685 | G | A | SBNO2 | p.T120M | 0 | 60 | 0.434783 | 46 | WCM10_Z1-1 | Tissue |
| 19 | 12502776 | G | A | ZNF799 | p.R146C | 0 | 163 | 0.358108 | 148 | WCM10_Z1-1 | Tissue |
| 19 | 14262296 | G | A | LPHN1 | p.R1272W | 0 | 146 | 0.344595 | 148 | WCM10_Z1-1 | Tissue |
| 19 | 1467081 | G | A | APC2 | p.V1261M | 0 | 104 | 0.333333 | 96 | WCM10_Z1-1 | Tissue |
| 19 | 15052875 | C | T | OR7C2 | p.T192I | 0 | 186 | 0.409091 | 154 | WCM10_Z1-1 | Tissue |
| 19 | 15278099 | C | T | NOTCH3 | p.A1775T | 0 | 84 | 0.081081 | 74 | WCM10_Z1-1 | Tissue |
| 19 | 18280055 | C | T | PIK3R2 | p.A713V | 0 | 100 | 0.247312 | 93 | WCM10_Z1-1 | Tissue |
| 19 | 2403116 | G | A | TMPRSS9 | p.S164N | 0 | 77 | 0.127273 | 55 | WCM10_Z1-1 | Tissue |
| 19 | 31767554 | T | C | TSHZ3 | p.T1049A | 0 | 155 | 0.310559 | 161 | WCM10_Z1-1 | Tissue |
| 19 | 34292106 | G | A | KCTD15 | p.R34Q | 0 | 277 | 0.314286 | 245 | WCM10_Z1-1 | Tissue |
| 19 | 35800969 | C | T | MAG | p.T475M | 0 | 71 | 0.104167 | 48 | WCM10_Z1-1 | Tissue |
| 19 | 36357071 | G | A | KIRREL2 | p.G602S | 0 | 92 | 0.272727 | 66 | WCM10_Z1-1 | Tissue |
| 19 | 3778366 | C | T | MATK | p.E447K | 0 | 172 | 0.321918 | 146 | WCM10_Z1-1 | Tissue |
| 19 | 38911560 | C | G | RASGRP4 | p.C122S | 0 | 206 | 0.342697 | 178 | WCM10_Z1-1 | Tissue |
| 19 | 38942479 | G | A | RYR1 | p.A400T | 0 | 117 | 0.311688 | 77 | WCM10_Z1-1 | Tissue |
| 19 | 38993310 | G | A | RYR1 | p.R2593H | 0 | 200 | 0.321951 | 205 | WCM10_Z1-1 | Tissue |
| 19 | 40424102 | C | T | FCGBP | p.V701I | 0 | 147 | 0.270492 | 122 | WCM10_Z1-1 | Tissue |
| 19 | 40839769 | C | T | C19orf47 | p.R134H | 0 | 76 | 0.509804 | 51 | WCM10_Z1-1 | Tissue |
| 19 | 44015647 | C | T | ETHE1 | p.M149I | 0 | 61 | 0.075472 | 53 | WCM10_Z1-1 | Tissue |
| 19 | 44131873 | C | T | CADM4 | p.R45H | 0 | 179 | 0.245902 | 122 | WCM10_Z1-1 | Tissue |
| 19 | 44933643 | C | T | ZNF229 | p.C438Y | 0 | 187 | 0.050847 | 177 | WCM10_Z1-1 | Tissue |
| 19 | 45855903 | C | T | ERCC2 | p.R636Q | 0 | 130 | 0.267327 | 101 | WCM10_Z1-1 | Tissue |
| 19 | 45917252 | G | A | ERCC1 | p.T248M | 0 | 47 | 0.45 | 40 | WCM10_Z1-1 | Tissue |
| 19 | 4793067 | G | A | FEM1A | p.A401T | 0 | 289 | 0.421687 | 249 | WCM10_Z1-1 | Tissue |
| 19 | 47941171 | C | T | SLC8A2 | p.G649S | 0 | 344 | 0.257812 | 256 | WCM10_Z1-1 | Tissue |
| 19 | 48660279 | C | T | LIG1 | p.R121H | 0 | 49 | 0.095238 | 42 | WCM10_Z1-1 | Tissue |
| 19 | 49685914 | G | A | TRPM4 | p.G448D | 0 | 361 | 0.275194 | 258 | WCM10_Z1-1 | Tissue |
| 19 | 50100585 | C | T | PRR12 | p.A998V | 0 | 258 | 0.361345 | 238 | WCM10_Z1-1 | Tissue |
| 19 | 50306616 | C | T | AP2A1 | p.P798L | 0 | 88 | 0.258065 | 93 | WCM10_Z1-1 | Tissue |
| 19 | 50376513 | C | T | AKT1S1 | p.V14M | 0 | 54 | 0.282609 | 46 | WCM10_Z1-1 | Tissue |
| 19 | 50957409 | G | A | MYBPC2 | p.V628M | 0 | 154 | 0.274336 | 113 | WCM10_Z1-1 | Tissue |
| 19 | 51848493 | G | A | ETFB | p.A247V | 0 | 126 | 0.370079 | 127 | WCM10_Z1-1 | Tissue |
| 19 | 52537238 | T | C | ZNF432 | p.E565G | 0 | 188 | 0.298246 | 171 | WCM10_Z1-1 | Tissue |
| 19 | 55591513 | A | G | EPS8L1 | p.Y99C | 0 | 64 | 0.223881 | 67 | WCM10_Z1-1 | Tissue |
| 19 | 55665537 | C | T | TNNI3 | p.G137D | 0 | 142 | 0.31068 | 103 | WCM10_Z1-1 | Tissue |
| 19 | 56133410 | C | T | ZNF784 | p.G227S | 0 | 44 | 0.243902 | 41 | WCM10_Z1-1 | Tissue |
| 19 | 56160780 | C | T | CCDC106 | p.P48L | 0 | 76 | 0.315789 | 95 | WCM10_Z1-1 | Tissue |
| 19 | 59009937 | C | T | SLC27A5 | p.R673Q | 0 | 246 | 0.349206 | 252 | WCM10_Z1-1 | Tissue |
| 19 | 59028640 | G | A | ZBTB45 | p.T134M | 0 | 39 | 0.291667 | 48 | WCM10_Z1-1 | Tissue |
| 19 | 59063511 | C | G | CHMP2A | p.E130D | 0 | 479 | 0.068293 | 410 | WCM10_Z1-1 | Tissue |
| 19 | 7546989 | C | T | PEX11G | p.V120M | 0 | 47 | 0.425926 | 54 | WCM10_Z1-1 | Tissue |
| 1 | 981932 | C | T | AGRN | p.R1023* | 0 | 146 | 0.443709 | 151 | WCM10_Z1-1 | Tissue |
| 19 | 8368768 | C | T | CD320 | p.C158Y | 0 | 49 | 0.333333 | 45 | WCM10_Z1-1 | Tissue |
| 19 | 9049889 | G | A | MUC16 | p.P10581L | 0 | 115 | 0.265487 | 113 | WCM10_Z1-1 | Tissue |
| 19 | 9065775 | G | A | MUC16 | p.A7224V | 0 | 182 | 0.322034 | 177 | WCM10_Z1-1 | Tissue |
| 20 | 17970739 | A | C | MGME1 | p.E341A | 0 | 124 | 0.35 | 120 | WCM10_Z1-1 | Tissue |
| 20 | 23028782 | C | T | THBD | p.V454M | 0 | 152 | 0.306748 | 163 | WCM10_Z1-1 | Tissue |
| 20 | 23345909 | C | T | GZF1 | p.P297S | 0 | 52 | 0.444444 | 45 | WCM10_Z1-1 | Tissue |
| 20 | 23346318 | G | A | GZF1 | p.R433Q | 0 | 59 | 0.3 | 60 | WCM10_Z1-1 | Tissue |
| 20 | 24949607 | G | A | APMAP | p.S321L | 0 | 118 | 0.364583 | 96 | WCM10_Z1-1 | Tissue |
| 20 | 30730869 | G | A | TM9SF4 | p.V205I | 0 | 153 | 0.346405 | 153 | WCM10_Z1-1 | Tissue |
| 20 | 31040712 | C | T | C20orf112 | p.R288H | 0 | 65 | 0.27451 | 51 | WCM10_Z1-1 | Tissue |
| 20 | 31628087 | C | T | BPIFB6 | p.H369Y | 0 | 259 | 0.097744 | 266 | WCM10_Z1-1 | Tissue |
| 20 | 31672750 | G | A | BPIFB4 | p.G244S | 0 | 158 | 0.257143 | 105 | WCM10_Z1-1 | Tissue |
| 20 | 33586362 | T | C | MYH7B | p.L1350P | 0 | 421 | 0.321526 | 367 | WCM10_Z1-1 | Tissue |
| 20 | 33703693 | G | A | EDEM2 | p.S427L | 0 | 158 | 0.345133 | 113 | WCM10_Z1-1 | Tissue |
| 20 | 36687868 | A | C | RPRD1B | p.I201L | 0 | 50 | 0.101695 | 59 | WCM10_Z1-1 | Tissue |
| 20 | 37536697 | C | A | PPP1R16B | p.S352* | 0 | 169 | 0.322785 | 158 | WCM10_Z1-1 | Tissue |
| 20 | 43804604 | C | T | PI3 | p.A61V | 0 | 94 | 0.389474 | 95 | WCM10_Z1-1 | Tissue |
| 20 | 44675057 | G | A | SLC12A5 | p.R613H | 0 | 140 | 0.1 | 140 | WCM10_Z1-1 | Tissue |
| 20 | 44803600 | G | A | CDH22 | p.Q678* | 0 | 130 | 0.07377 | 122 | WCM10_Z1-1 | Tissue |
| 20 | 45354634 | C | T | SLC2A10 | p.A320V | 0 | 129 | 0.378151 | 119 | WCM10_Z1-1 | Tissue |
| 20 | 47246057 | G | A | PREX1 | p.P1566S | 0 | 47 | 0.314286 | 35 | WCM10_Z1-1 | Tissue |
| 20 | 4843432 | G | A | SLC23A2 | p.T493M | 0 | 43 | 0.487179 | 39 | WCM10_Z1-1 | Tissue |
| 20 | 57429071 | G | A | GNAS | p.G251S | 0 | 328 | 0.073955 | 311 | WCM10_Z1-1 | Tissue |
| 20 | 62329713 | C | T | TNFRSF6B | p.Q234* | 0 | 145 | 0.42953 | 149 | WCM10_Z1-1 | Tissue |
| 20 | 62729869 | C | T | OPRL1 | p.T277M | 0 | 179 | 0.083871 | 155 | WCM10_Z1-1 | Tissue |
| 20 | 62839109 | C | T | MYT1 | p.A187V | 0 | 146 | 0.345588 | 136 | WCM10_Z1-1 | Tissue |
| 20 | 7976714 | A | G | TMX4 | p.S158P | 0 | 53 | 0.391304 | 46 | WCM10_Z1-1 | Tissue |
| 2 | 111395549 | G | A | BUB1 | p.R1084* | 0 | 110 | 0.08209 | 134 | WCM10_Z1-1 | Tissue |
| 2 | 116447300 | A | G | DPP10 | p.Y160C | 0 | 36 | 0.095238 | 42 | WCM10_Z1-1 | Tissue |
| 2 | 121992810 | C | T | TFCP2L1 | p.A361T | 0 | 160 | 0.286713 | 143 | WCM10_Z1-1 | Tissue |
| 21 | 31971103 | G | A | KRTAP6-2 | p.R31C | 0 | 149 | 0.314685 | 143 | WCM10_Z1-1 | Tissue |
| 21 | 40649199 | A | T | BRWD1 | p.I361N | 0 | 80 | 0.25 | 64 | WCM10_Z1-1 | Tissue |
| 21 | 43905882 | G | A | RSPH1 | p.T133M | 0 | 140 | 0.314286 | 140 | WCM10_Z1-1 | Tissue |
| 21 | 46702336 | T | C | POFUT2 | p.N189S | 0 | 76 | 0.09 | 100 | WCM10_Z1-1 | Tissue |
| 21 | 47678923 | C | T | MCM3AP | p.V1222M | 0 | 63 | 0.415385 | 65 | WCM10_Z1-1 | Tissue |
| 21 | 47836333 | T | A | PCNT | p.D2167E | 0 | 124 | 0.308411 | 107 | WCM10_Z1-1 | Tissue |
| 2 | 153526877 | G | A | PRPF40A | p.R500* | 0 | 31 | 0.274194 | 62 | WCM10_Z1-1 | Tissue |
| 2 | 15555683 | T | C | NBAS | p.H975R | 0 | 49 | 0.387755 | 49 | WCM10_Z1-1 | Tissue |
| 2 | 15601391 | G | A | NBAS | p.A758V | 0 | 42 | 0.105263 | 38 | WCM10_Z1-1 | Tissue |
| 2 | 168098390 | A | C | XIRP2 | p.K415N | 0 | 62 | 0.303571 | 56 | WCM10_Z1-1 | Tissue |
| 2 | 170062573 | C | T | LRP2 | p.V2506I | 0 | 84 | 0.377551 | 98 | WCM10_Z1-1 | Tissue |
| 2 | 172935684 | G | A | METAP1D | p.G197D | 0 | 46 | 0.321429 | 56 | WCM10_Z1-1 | Tissue |
| 2 | 177016750 | C | T | HOXD4 | p.P130L | 0 | 98 | 0.07 | 100 | WCM10_Z1-1 | Tissue |
| 2 | 178416834 | G | A | TTC30B | p.R220C | 0 | 231 | 0.30292 | 274 | WCM10_Z1-1 | Tissue |
| 2 | 179403742 | C | T | TTN | p.A31333T | 0 | 203 | 0.050228 | 219 | WCM10_Z1-1 | Tissue |
| 2 | 183094761 | T | A | PDE1A | p.H232L | 0 | 96 | 0.258065 | 93 | WCM10_Z1-1 | Tissue |
| 2 | 192711200 | C | T | SDPR | p.R151Q | 0 | 130 | 0.29703 | 101 | WCM10_Z1-1 | Tissue |
| 2 | 196674542 | C | T | DNAH7 | p.R3272Q | 0 | 61 | 0.305556 | 72 | WCM10_Z1-1 | Tissue |
| 2 | 196740527 | A | G | DNAH7 | p.V2053A | 0 | 170 | 0.286517 | 178 | WCM10_Z1-1 | Tissue |
| 2 | 211542647 | C | T | CPS1 | p.R1481C | 0 | 76 | 0.325 | 80 | WCM10_Z1-1 | Tissue |
| 2 | 219514122 | C | T | ZNF142 | p.R170H | 0 | 48 | 0.3 | 60 | WCM10_Z1-1 | Tissue |
| 2 | 219612433 | G | A | TTLL4 | p.R788H | 0 | 75 | 0.305556 | 72 | WCM10_Z1-1 | Tissue |
| 2 | 220290430 | C | T | DES | p.T445M | 0 | 102 | 0.253165 | 79 | WCM10_Z1-1 | Tissue |
| 2 | 220397070 | C | T | ASIC4 | p.R424C | 0 | 83 | 0.071429 | 84 | WCM10_Z1-1 | Tissue |
| 2 | 220397157 | G | A | ASIC4 | p.A453T | 0 | 62 | 0.455882 | 68 | WCM10_Z1-1 | Tissue |
| 22 | 26304313 | A | T | MYO18B | p.I1726F | 0 | 37 | 0.34375 | 32 | WCM10_Z1-1 | Tissue |
| 2 | 228159737 | G | A | COL4A3 | p.R1159H | 0 | 140 | 0.393443 | 122 | WCM10_Z1-1 | Tissue |
| 22 | 31534686 | A | G | PLA2G3 | p.F205S | 0 | 99 | 0.25974 | 77 | WCM10_Z1-1 | Tissue |
| 22 | 31998611 | C | T | SFI1 | p.R549* | 0 | 180 | 0.283019 | 159 | WCM10_Z1-1 | Tissue |
| 2 | 233273223 | G | A | ALPPL2 | p.V266M | 0 | 102 | 0.351351 | 74 | WCM10_Z1-1 | Tissue |
| 2 | 233274365 | G | A | ALPPL2 | p.R461H | 0 | 94 | 0.097222 | 72 | WCM10_Z1-1 | Tissue |
| 22 | 35815891 | G | A | MCM5 | p.R573Q | 0 | 109 | 0.375 | 96 | WCM10_Z1-1 | Tissue |
| 22 | 36698672 | C | T | MYH9 | p.R814Q | 0 | 38 | 0.340909 | 44 | WCM10_Z1-1 | Tissue |
| 22 | 37603395 | G | A | SSTR3 | p.R150C | 0 | 72 | 0.0625 | 64 | WCM10_Z1-1 | Tissue |
| 22 | 38219447 | C | T | GALR3 | p.P12S | 0 | 123 | 0.28125 | 96 | WCM10_Z1-1 | Tissue |
| 2 | 239038950 | G | A | ESPNL | p.R532Q | 0 | 56 | 0.076923 | 52 | WCM10_Z1-1 | Tissue |
| 22 | 40417472 | C | T | FAM83F | p.R320* | 0 | 330 | 0.305147 | 272 | WCM10_Z1-1 | Tissue |
| 2 | 240969191 | C | G | OR6B2 | p.W219S | 0 | 124 | 0.098039 | 102 | WCM10_Z1-1 | Tissue |
| 2 | 240969192 | A | C | OR6B2 | p.W219G | 0 | 123 | 0.1 | 100 | WCM10_Z1-1 | Tissue |
| 2 | 242501811 | G | A | BOK | p.R90H | 0 | 87 | 0.108108 | 111 | WCM10_Z1-1 | Tissue |
| 22 | 42525748 | C | T | CYP2D6 | p.R115H | 0 | 81 | 0.368421 | 76 | WCM10_Z1-1 | Tissue |
| 22 | 43568479 | C | T | TTLL12 | p.V475M | 0 | 300 | 0.330677 | 251 | WCM10_Z1-1 | Tissue |
| 22 | 46733744 | G | A | TRMU | p.D51N | 0 | 138 | 0.308333 | 120 | WCM10_Z1-1 | Tissue |
| 22 | 46859820 | G | A | CELSR1 | p.R1323* | 0 | 331 | 0.329114 | 237 | WCM10_Z1-1 | Tissue |
| 2 | 25057678 | C | T | ADCY3 | p.R597Q | 0 | 145 | 0.339806 | 103 | WCM10_Z1-1 | Tissue |
| 22 | 51159328 | C | T | SHANK3 | p.R1009W | 0 | 87 | 0.262295 | 61 | WCM10_Z1-1 | Tissue |
| 2 | 26536276 | G | A | GPR113 | p.A481V | 0 | 56 | 0.316667 | 60 | WCM10_Z1-1 | Tissue |
| 2 | 27880243 | C | T | SUPT7L | p.R238H | 0.002976 | 336 | 0.057057 | 333 | WCM10_Z1-1 | Tissue |
| 2 | 29416527 | C | T | ALK | p.V1476M | 0 | 160 | 0.335443 | 158 | WCM10_Z1-1 | Tissue |
| 2 | 36739445 | G | A | CRIM1 | p.R563H | 0 | 72 | 0.318182 | 66 | WCM10_Z1-1 | Tissue |
| 2 | 39050359 | G | A | DHX57 | p.R1023W | 0 | 47 | 0.378378 | 37 | WCM10_Z1-1 | Tissue |
| 2 | 48030670 | G | A | MSH6 | p.R1095H | 0 | 368 | 0.05314 | 207 | WCM10_Z1-1 | Tissue |
| 2 | 63283204 | C | T | OTX1 | p.A273V | 0 | 196 | 0.053254 | 169 | WCM10_Z1-1 | Tissue |
| 2 | 70057092 | C | T | GMCL1 | p.A23V | 0 | 119 | 0.425926 | 108 | WCM10_Z1-1 | Tissue |
| 2 | 77746838 | C | T | LRRTM4 | p.A53T | 0 | 240 | 0.364964 | 274 | WCM10_Z1-1 | Tissue |
| 2 | 85766496 | A | G | MAT2A | p.H29R | 0 | 94 | 0.050633 | 79 | WCM10_Z1-1 | Tissue |
| 2 | 86302239 | C | T | POLR1A | p.A509T | 0 | 85 | 0.352273 | 88 | WCM10_Z1-1 | Tissue |
| 2 | 98373636 | G | A | TMEM131 | p.R1860W | 0 | 184 | 0.348148 | 135 | WCM10_Z1-1 | Tissue |
| 3 | 108355532 | G | A | DZIP3 | p.V330I | 0 | 85 | 0.35 | 80 | WCM10_Z1-1 | Tissue |
| 3 | 108672424 | A | T | GUCA1C | p.N62K | 0 | 77 | 0.054348 | 92 | WCM10_Z1-1 | Tissue |
| 3 | 113377122 | G | A | KIAA2018 | p.A1136V | 0 | 90 | 0.11236 | 89 | WCM10_Z1-1 | Tissue |
| 3 | 114069661 | G | A | ZBTB20 | p.Q422* | 0 | 178 | 0.351852 | 162 | WCM10_Z1-1 | Tissue |
| 3 | 119133387 | G | A | ARHGAP31 | p.V871I | 0 | 170 | 0.301775 | 169 | WCM10_Z1-1 | Tissue |
| 3 | 119150871 | G | A | TMEM39A | p.A475V | 0 | 51 | 0.333333 | 48 | WCM10_Z1-1 | Tissue |
| 3 | 123008728 | C | T | ADCY5 | p.G1134D | 0 | 61 | 0.306452 | 62 | WCM10_Z1-1 | Tissue |
| 3 | 129373876 | G | A | TMCC1 | p.Q528* | 0 | 61 | 0.465116 | 43 | WCM10_Z1-1 | Tissue |
| 3 | 129389857 | C | T | TMCC1 | p.R276H | 0 | 403 | 0.295775 | 355 | WCM10_Z1-1 | Tissue |
| 3 | 138478044 | G | A | PIK3CB | p.R48W | 0 | 47 | 0.28125 | 32 | WCM10_Z1-1 | Tissue |
| 3 | 143550991 | C | T | SLC9A9 | p.C83Y | 0 | 52 | 0.396552 | 58 | WCM10_Z1-1 | Tissue |
| 3 | 150690462 | T | C | CLRN1 | p.M12V | 0 | 273 | 0.296429 | 280 | WCM10_Z1-1 | Tissue |
| 3 | 151176331 | G | A | IGSF10 | p.P56L | 0 | 47 | 0.368421 | 38 | WCM10_Z1-1 | Tissue |
| 3 | 152554031 | G | A | P2RY1 | p.V154M | 0 | 173 | 0.329949 | 197 | WCM10_Z1-1 | Tissue |
| 3 | 155232543 | G | A | PLCH1 | p.T522M | 0 | 119 | 0.365385 | 104 | WCM10_Z1-1 | Tissue |
| 3 | 16216789 | T | C | GALNT15 | p.V44A | 0 | 226 | 0.310526 | 190 | WCM10_Z1-1 | Tissue |
| 3 | 170015104 | C | T | PRKCI | p.R504* | 0 | 56 | 0.276596 | 47 | WCM10_Z1-1 | Tissue |
| 3 | 172165797 | G | A | GHSR | p.A136V | 0 | 111 | 0.271028 | 107 | WCM10_Z1-1 | Tissue |
| 3 | 178916623 | C | T | PIK3CA | p.R4* | 0 | 39 | 0.088889 | 45 | WCM10_Z1-1 | Tissue |
| 3 | 194118399 | G | A | GP5 | p.R205C | 0 | 162 | 0.373016 | 126 | WCM10_Z1-1 | Tissue |
| 3 | 195495927 | G | A | MUC4 | p.R263C | 0 | 95 | 0.376471 | 85 | WCM10_Z1-1 | Tissue |
| 3 | 32491039 | G | A | CMTM7 | p.G143R | 0 | 36 | 0.212121 | 33 | WCM10_Z1-1 | Tissue |
| 3 | 33415149 | C | T | FBXL2 | p.R179* | 0 | 83 | 0.349057 | 106 | WCM10_Z1-1 | Tissue |
| 3 | 38167177 | G | A | ACAA1 | p.R360* | 0 | 81 | 0.280488 | 82 | WCM10_Z1-1 | Tissue |
| 3 | 391116 | G | A | CHL1 | p.G308D | 0 | 82 | 0.28 | 75 | WCM10_Z1-1 | Tissue |
| 3 | 45266896 | C | A | TMEM158 | p.Q208H | 0 | 127 | 0.330645 | 124 | WCM10_Z1-1 | Tissue |
| 3 | 48465594 | C | T | PLXNB1 | p.V143M | 0 | 115 | 0.304348 | 92 | WCM10_Z1-1 | Tissue |
| 3 | 48511138 | C | T | SHISA5 | p.R140H | 0 | 51 | 0.309091 | 55 | WCM10_Z1-1 | Tissue |
| 3 | 48694506 | C | T | CELSR3 | p.A1342T | 0 | 334 | 0.347134 | 314 | WCM10_Z1-1 | Tissue |
| 3 | 49701094 | G | T | BSN | p.G3835* | 0 | 158 | 0.310345 | 145 | WCM10_Z1-1 | Tissue |
| 3 | 51697135 | C | T | RAD54L2 | p.S1368F | 0 | 320 | 0.348123 | 293 | WCM10_Z1-1 | Tissue |
| 3 | 54596912 | A | G | CACNA2D3 | p.I210M | 0 | 54 | 0.25 | 64 | WCM10_Z1-1 | Tissue |
| 3 | 64085133 | C | T | PRICKLE2 | p.R710Q | 0 | 128 | 0.072581 | 124 | WCM10_Z1-1 | Tissue |
| 3 | 64145684 | G | A | PRICKLE2 | p.R110C | 0 | 121 | 0.431579 | 95 | WCM10_Z1-1 | Tissue |
| 3 | 64579981 | G | A | ADAMTS9 | p.H1437Y | 0 | 127 | 0.267123 | 146 | WCM10_Z1-1 | Tissue |
| 3 | 7620917 | T | C | GRM7 | p.I775T | 0 | 89 | 0.297619 | 84 | WCM10_Z1-1 | Tissue |
| 3 | 9991622 | C | T | PRRT3 | p.V60M | 0 | 106 | 0.415094 | 106 | WCM10_Z1-1 | Tissue |
| 4 | 10099441 | C | T | WDR1 | p.S151N | 0 | 89 | 0.123077 | 65 | WCM10_Z1-1 | Tissue |
| 4 | 1018278 | G | A | FGFRL1 | p.G300S | 0 | 183 | 0.051948 | 154 | WCM10_Z1-1 | Tissue |
| 4 | 106863686 | C | A | NPNT | p.P329Q | 0 | 485 | 0.078704 | 432 | WCM10_Z1-1 | Tissue |
| 4 | 107157932 | G | A | TBCK | p.P402S | 0 | 62 | 0.058824 | 51 | WCM10_Z1-1 | Tissue |
| 4 | 121675739 | C | T | PRDM5 | p.G531E | 0 | 85 | 0.594203 | 69 | WCM10_Z1-1 | Tissue |
| 4 | 123237963 | G | A | KIAA1109 | p.R3539H | 0 | 33 | 0.128205 | 39 | WCM10_Z1-1 | Tissue |
| 4 | 141578691 | C | T | TBC1D9 | p.V733I | 0 | 84 | 0.238636 | 88 | WCM10_Z1-1 | Tissue |
| 4 | 147561160 | A | C | POU4F2 | p.T144P | 0 | 288 | 0.336879 | 282 | WCM10_Z1-1 | Tissue |
| 4 | 170322839 | T | C | NEK1 | p.S1155G | 0 | 106 | 0.398058 | 103 | WCM10_Z1-1 | Tissue |
| 4 | 183714417 | C | T | TENM3 | p.R2198C | 0 | 214 | 0.315789 | 228 | WCM10_Z1-1 | Tissue |
| 4 | 185018479 | G | A | ENPP6 | p.R346W | 0 | 104 | 0.275862 | 87 | WCM10_Z1-1 | Tissue |
| 4 | 1957897 | G | A | WHSC1 | p.G955R | 0 | 65 | 0.37931 | 58 | WCM10_Z1-1 | Tissue |
| 4 | 2097629 | C | T | POLN | p.A672T | 0 | 149 | 0.300578 | 173 | WCM10_Z1-1 | Tissue |
| 4 | 2692544 | A | G | FAM193A | p.K593E | 0 | 102 | 0.3125 | 80 | WCM10_Z1-1 | Tissue |
| 4 | 41621352 | C | T | LIMCH1 | p.T277M | 0 | 136 | 0.089552 | 134 | WCM10_Z1-1 | Tissue |
| 4 | 42554524 | G | A | ATP8A1 | p.P506L | 0 | 70 | 0.180556 | 72 | WCM10_Z1-1 | Tissue |
| 4 | 4317388 | C | T | ZBTB49 | p.R468C | 0 | 71 | 0.263158 | 76 | WCM10_Z1-1 | Tissue |
| 4 | 56841100 | C | T | CEP135 | p.R480C | 0 | 91 | 0.372093 | 86 | WCM10_Z1-1 | Tissue |
| 4 | 72423507 | C | T | SLC4A4 | p.R948C | 0 | 69 | 0.276316 | 76 | WCM10_Z1-1 | Tissue |
| 4 | 74283811 | G | A | ALB | p.V329M | 0 | 73 | 0.236559 | 93 | WCM10_Z1-1 | Tissue |
| 4 | 78085559 | C | A | CCNG2 | p.L280I | 0 | 266 | 0.285714 | 238 | WCM10_Z1-1 | Tissue |
| 4 | 8621109 | G | A | CPZ | p.G575D | 0.004566 | 219 | 0.340782 | 179 | WCM10_Z1-1 | Tissue |
| 4 | 90857691 | C | T | MMRN1 | p.P954S | 0 | 182 | 0.058252 | 206 | WCM10_Z1-1 | Tissue |
| 4 | 947036 | G | A | TMEM175 | p.R174H | 0 | 180 | 0.278571 | 140 | WCM10_Z1-1 | Tissue |
| 5 | 102342673 | C | T | PAM | p.Q658* | 0 | 79 | 0.176471 | 34 | WCM10_Z1-1 | Tissue |
| 5 | 115818186 | C | T | SEMA6A | p.D350N | 0 | 54 | 0.085714 | 35 | WCM10_Z1-1 | Tissue |
| 5 | 127622532 | T | C | FBN2 | p.E2297G | 0 | 43 | 0.096774 | 31 | WCM10_Z1-1 | Tissue |
| 5 | 131924481 | G | A | RAD50 | p.R385H | 0 | 42 | 0.323529 | 34 | WCM10_Z1-1 | Tissue |
| 5 | 132158730 | C | T | SHROOM1 | p.V773M | 0 | 118 | 0.342857 | 105 | WCM10_Z1-1 | Tissue |
| 5 | 140189003 | C | T | PCDHA4 | p.A744V | 0 | 83 | 0.317647 | 85 | WCM10_Z1-1 | Tissue |
| 5 | 140202321 | G | A | PCDHA5 | p.A321T | 0 | 151 | 0.299363 | 157 | WCM10_Z1-1 | Tissue |
| 5 | 140730012 | G | A | PCDHGB1 | p.R62Q | 0.007143 | 140 | 0.322368 | 152 | WCM10_Z1-1 | Tissue |
| 5 | 140871065 | C | T | PCDHGC5 | p.S753L | 0 | 229 | 0.27957 | 186 | WCM10_Z1-1 | Tissue |
| 5 | 145610364 | C | A | RBM27 | p.P245H | 0 | 114 | 0.066667 | 105 | WCM10_Z1-1 | Tissue |
| 5 | 149582170 | G | A | SLC6A7 | p.A331T | 0 | 36 | 0.22807 | 57 | WCM10_Z1-1 | Tissue |
| 5 | 157078477 | G | T | SOX30 | p.P204T | 0 | 454 | 0.297436 | 390 | WCM10_Z1-1 | Tissue |
| 5 | 175913360 | C | T | FAF2 | p.A46V | 0 | 313 | 0.325088 | 283 | WCM10_Z1-1 | Tissue |
| 5 | 176025146 | A | G | GPRIN1 | p.S564P | 0 | 248 | 0.356863 | 255 | WCM10_Z1-1 | Tissue |
| 5 | 178771118 | C | T | ADAMTS2 | p.V62M | 0 | 108 | 0.271845 | 103 | WCM10_Z1-1 | Tissue |
| 5 | 31410883 | G | A | DROSHA | p.R1213C | 0 | 121 | 0.368 | 125 | WCM10_Z1-1 | Tissue |
| 5 | 35876433 | G | T | IL7R | p.G409W | 0 | 150 | 0.337209 | 172 | WCM10_Z1-1 | Tissue |
| 5 | 38484884 | G | A | LIFR | p.R862* | 0 | 253 | 0.341772 | 237 | WCM10_Z1-1 | Tissue |
| 5 | 38490385 | G | A | LIFR | p.R692* | 0 | 73 | 0.343284 | 67 | WCM10_Z1-1 | Tissue |
| 5 | 39376950 | C | G | DAB2 | p.E647Q | 0 | 118 | 0.262712 | 118 | WCM10_Z1-1 | Tissue |
| 5 | 50073914 | C | T | PARP8 | p.A177V | 0 | 31 | 0.159091 | 44 | WCM10_Z1-1 | Tissue |
| 5 | 53814512 | G | A | SNX18 | p.V244M | 0 | 161 | 0.274074 | 135 | WCM10_Z1-1 | Tissue |
| 5 | 55110902 | G | A | DDX4 | p.R630H | 0.005952 | 168 | 0.075949 | 158 | WCM10_Z1-1 | Tissue |
| 5 | 55422845 | C | T | ANKRD55 | p.C234Y | 0 | 130 | 0.300971 | 103 | WCM10_Z1-1 | Tissue |
| 5 | 63510253 | G | A | RNF180 | p.S367N | 0 | 66 | 0.257576 | 66 | WCM10_Z1-1 | Tissue |
| 5 | 79336091 | C | T | THBS4 | p.R94C | 0 | 143 | 0.325 | 120 | WCM10_Z1-1 | Tissue |
| 5 | 89769683 | C | T | MBLAC2 | p.V143M | 0 | 68 | 0.256757 | 74 | WCM10_Z1-1 | Tissue |
| 5 | 89953815 | C | T | GPR98 | p.T1491M | 0 | 61 | 0.118644 | 59 | WCM10_Z1-1 | Tissue |
| 5 | 94024218 | C | T | ANKRD32 | p.S710F | 0 | 119 | 0.309735 | 113 | WCM10_Z1-1 | Tissue |
| 6 | 112671082 | G | A | RFPL4B | p.V58I | 0 | 307 | 0.054913 | 346 | WCM10_Z1-1 | Tissue |
| 6 | 128134119 | C | T | THEMIS | p.R556H | 0 | 119 | 0.384615 | 130 | WCM10_Z1-1 | Tissue |
| 6 | 129419331 | C | T | LAMA2 | p.A137V | 0 | 199 | 0.257642 | 229 | WCM10_Z1-1 | Tissue |
| 6 | 136562753 | G | A | MTFR2 | p.R115W | 0 | 108 | 0.385542 | 83 | WCM10_Z1-1 | Tissue |
| 6 | 1390541 | G | A | FOXF2 | p.R120H | 0 | 142 | 0.274809 | 131 | WCM10_Z1-1 | Tissue |
| 6 | 149922784 | C | G | KATNA1 | p.L278F | 0 | 74 | 0.283582 | 67 | WCM10_Z1-1 | Tissue |
| 6 | 152603061 | A | G | SYNE1 | p.Y6088H | 0 | 146 | 0.285714 | 147 | WCM10_Z1-1 | Tissue |
| 6 | 158924441 | C | T | TULP4 | p.T1249M | 0 | 161 | 0.370861 | 151 | WCM10_Z1-1 | Tissue |
| 6 | 160465609 | T | A | IGF2R | p.I562N | 0 | 60 | 0.09434 | 53 | WCM10_Z1-1 | Tissue |
| 6 | 166778930 | G | A | MPC1 | p.T106M | 0 | 73 | 0.300885 | 113 | WCM10_Z1-1 | Tissue |
| 6 | 17675875 | G | A | NUP153 | p.S154L | 0 | 111 | 0.336957 | 92 | WCM10_Z1-1 | Tissue |
| 6 | 24146040 | C | T | NRSN1 | p.Q152* | 0 | 191 | 0.349462 | 186 | WCM10_Z1-1 | Tissue |
| 6 | 26087679 | G | A | HFE | p.R4Q | 0 | 51 | 0.240741 | 54 | WCM10_Z1-1 | Tissue |
| 6 | 26189262 | C | T | HIST1H4D | p.G15S | 0 | 155 | 0.339286 | 112 | WCM10_Z1-1 | Tissue |
| 6 | 26250728 | C | T | HIST1H3F | p.V36M | 0 | 153 | 0.315068 | 146 | WCM10_Z1-1 | Tissue |
| 6 | 28227352 | G | A | NKAPL | p.R68H | 0 | 207 | 0.302439 | 205 | WCM10_Z1-1 | Tissue |
| 6 | 28251733 | G | A | PGBD1 | p.R48Q | 0 | 106 | 0.347458 | 118 | WCM10_Z1-1 | Tissue |
| 6 | 29796440 | G | A | HLA-G | p.R155H | 0 | 92 | 0.41 | 100 | WCM10_Z1-1 | Tissue |
| 6 | 30139980 | G | A | TRIM15 | p.A418T | 0 | 77 | 0.314607 | 89 | WCM10_Z1-1 | Tissue |
| 6 | 31599491 | C | T | PRRC2A | p.S1014L | 0 | 110 | 0.386139 | 101 | WCM10_Z1-1 | Tissue |
| 6 | 31807429 | C | T | C6orf48 | p.P73S | 0 | 82 | 0.285714 | 84 | WCM10_Z1-1 | Tissue |
| 6 | 32023768 | C | T | TNXB | p.R2776Q | 0 | 99 | 0.207317 | 82 | WCM10_Z1-1 | Tissue |
| 6 | 32035513 | G | A | TNXB | p.R2157C | 0 | 119 | 0.412371 | 97 | WCM10_Z1-1 | Tissue |
| 6 | 32165134 | C | T | NOTCH4 | p.R1665Q | 0 | 47 | 0.345455 | 55 | WCM10_Z1-1 | Tissue |
| 6 | 32810026 | C | T | PSMB8 | p.R141Q | 0 | 95 | 0.366337 | 101 | WCM10_Z1-1 | Tissue |
| 6 | 36990084 | C | T | FGD2 | p.R466C | 0 | 203 | 0.357143 | 168 | WCM10_Z1-1 | Tissue |
| 6 | 4935880 | C | T | CDYL | p.R329C | 0 | 224 | 0.292308 | 260 | WCM10_Z1-1 | Tissue |
| 6 | 51720792 | G | A | PKHD1 | p.R2604C | 0 | 77 | 0.22093 | 86 | WCM10_Z1-1 | Tissue |
| 6 | 56420328 | T | C | DST | p.N4773S | 0 | 378 | 0.298578 | 422 | WCM10_Z1-1 | Tissue |
| 6 | 6006948 | A | G | NRN1 | p.L12P | 0 | 160 | 0.338235 | 136 | WCM10_Z1-1 | Tissue |
| 6 | 73905087 | G | A | KCNQ5 | p.A917T | 0 | 107 | 0.329412 | 85 | WCM10_Z1-1 | Tissue |
| 6 | 74073548 | C | T | KHDC3L | p.R207C | 0 | 160 | 0.336066 | 122 | WCM10_Z1-1 | Tissue |
| 6 | 83845403 | C | T | DOPEY1 | p.A979V | 0 | 129 | 0.309091 | 110 | WCM10_Z1-1 | Tissue |
| 6 | 88046835 | G | A | SMIM8 | p.R29H | 0 | 91 | 0.063158 | 95 | WCM10_Z1-1 | Tissue |
| 6 | 90415872 | A | G | MDN1 | p.V2685A | 0 | 32 | 0.413043 | 46 | WCM10_Z1-1 | Tissue |
| 7 | 100187924 | G | A | FBXO24 | p.R89H | 0 | 121 | 0.394958 | 119 | WCM10_Z1-1 | Tissue |
| 7 | 102962501 | G | A | DNAJC2 | p.R321W | 0 | 167 | 0.064865 | 185 | WCM10_Z1-1 | Tissue |
| 7 | 123302371 | C | T | LMOD2 | p.T244M | 0 | 124 | 0.058394 | 137 | WCM10_Z1-1 | Tissue |
| 7 | 129311325 | C | T | NRF1 | p.P94S | 0 | 136 | 0.373016 | 126 | WCM10_Z1-1 | Tissue |
| 7 | 135392952 | A | C | SLC13A4 | p.V92G | 0 | 42 | 0.265306 | 49 | WCM10_Z1-1 | Tissue |
| 7 | 137792181 | C | A | AKR1D1 | p.P237H | 0 | 68 | 0.076923 | 78 | WCM10_Z1-1 | Tissue |
| 7 | 140481402 | C | G | BRAF | p.G469A | 0 | 68 | 0.38 | 50 | WCM10_Z1-1 | Tissue |
| 7 | 150768788 | A | G | SLC4A2 | p.Q735R | 0 | 125 | 0.344 | 125 | WCM10_Z1-1 | Tissue |
| 7 | 150920903 | G | A | ABCF2 | p.R195C | 0 | 70 | 0.113208 | 53 | WCM10_Z1-1 | Tissue |
| 7 | 155251326 | C | T | EN2 | p.A85V | 0 | 111 | 0.306931 | 101 | WCM10_Z1-1 | Tissue |
| 7 | 16817450 | A | G | TSPAN13 | p.N114D | 0 | 88 | 0.236111 | 72 | WCM10_Z1-1 | Tissue |
| 7 | 21611581 | G | A | DNAH11 | p.C528Y | 0 | 60 | 0.219178 | 73 | WCM10_Z1-1 | Tissue |
| 7 | 23163451 | G | A | KLHL7 | p.R59H | 0 | 43 | 0.282609 | 46 | WCM10_Z1-1 | Tissue |
| 7 | 23349052 | T | C | MALSU1 | p.Y199H | 0 | 202 | 0.294479 | 163 | WCM10_Z1-1 | Tissue |
| 7 | 2419094 | G | A | EIF3B | p.V803I | 0 | 86 | 0.068182 | 88 | WCM10_Z1-1 | Tissue |
| 7 | 2473242 | G | A | CHST12 | p.R323Q | 0 | 242 | 0.341709 | 199 | WCM10_Z1-1 | Tissue |
| 7 | 28848933 | C | T | CREB5 | p.R386W | 0 | 69 | 0.39726 | 73 | WCM10_Z1-1 | Tissue |
| 7 | 30893019 | C | T | FAM188B | p.H541Y | 0 | 34 | 0.358974 | 39 | WCM10_Z1-1 | Tissue |
| 7 | 34724255 | A | G | NPSR1 | p.K80R | 0 | 44 | 0.40678 | 59 | WCM10_Z1-1 | Tissue |
| 7 | 42004916 | G | A | GLI3 | p.P1252L | 0 | 144 | 0.394558 | 147 | WCM10_Z1-1 | Tissue |
| 7 | 4218134 | C | T | SDK1 | p.R1672C | 0 | 43 | 0.288889 | 45 | WCM10_Z1-1 | Tissue |
| 7 | 44260473 | G | A | CAMK2B | p.T542M | 0 | 90 | 0.417582 | 91 | WCM10_Z1-1 | Tissue |
| 7 | 45004618 | C | T | MYO1G | p.A818T | 0 | 80 | 0.293103 | 58 | WCM10_Z1-1 | Tissue |
| 7 | 45139949 | G | A | TBRG4 | p.A624V | 0 | 74 | 0.411765 | 68 | WCM10_Z1-1 | Tissue |
| 7 | 47408037 | C | T | TNS3 | p.V736M | 0 | 80 | 0.292683 | 82 | WCM10_Z1-1 | Tissue |
| 7 | 48314164 | T | C | ABCA13 | p.L1634P | 0 | 227 | 0.058608 | 273 | WCM10_Z1-1 | Tissue |
| 7 | 590153 | G | A | PRKAR1B | p.R354C | 0 | 79 | 0.357143 | 70 | WCM10_Z1-1 | Tissue |
| 7 | 72907233 | G | A | BAZ1B | p.S197L | 0 | 36 | 0.096774 | 31 | WCM10_Z1-1 | Tissue |
| 7 | 75197525 | G | A | HIP1 | p.R261C | 0 | 116 | 0.060976 | 82 | WCM10_Z1-1 | Tissue |
| 7 | 780511 | G | A | HEATR2 | p.R279H | 0 | 198 | 0.393064 | 173 | WCM10_Z1-1 | Tissue |
| 7 | 8198266 | C | T | ICA1 | p.R199H | 0 | 82 | 0.053333 | 75 | WCM10_Z1-1 | Tissue |
| 7 | 97874361 | C | T | TECPR1 | p.G82S | 0 | 83 | 0.363636 | 88 | WCM10_Z1-1 | Tissue |
| 8 | 100396522 | C | T | VPS13B | p.R971* | 0 | 47 | 0.4 | 35 | WCM10_Z1-1 | Tissue |
| 8 | 119391874 | G | A | SAMD12 | p.R130W | 0 | 200 | 0.35 | 200 | WCM10_Z1-1 | Tissue |
| 8 | 123965008 | C | T | ZHX2 | p.R420C | 0 | 324 | 0.276923 | 260 | WCM10_Z1-1 | Tissue |
| 8 | 125565703 | G | A | MTSS1 | p.R600C | 0 | 112 | 0.368 | 125 | WCM10_Z1-1 | Tissue |
| 8 | 136619213 | T | A | KHDRBS3 | p.Y275N | 0 | 144 | 0.344156 | 154 | WCM10_Z1-1 | Tissue |
| 8 | 144809865 | C | T | FAM83H | p.G589D | 0 | 84 | 0.36 | 75 | WCM10_Z1-1 | Tissue |
| 8 | 144919600 | C | T | NRBP2 | p.R352H | 0 | 231 | 0.073276 | 232 | WCM10_Z1-1 | Tissue |
| 8 | 144992347 | C | T | PLEC | p.R4018H | 0 | 64 | 0.424658 | 73 | WCM10_Z1-1 | Tissue |
| 8 | 145112410 | T | C | OPLAH | p.M455V | 0 | 52 | 0.195652 | 46 | WCM10_Z1-1 | Tissue |
| 8 | 20068085 | G | A | ATP6V1B2 | p.V131I | 0 | 147 | 0.354839 | 186 | WCM10_Z1-1 | Tissue |
| 8 | 23056948 | C | T | TNFRSF10A | p.R282H | 0 | 42 | 0.212121 | 33 | WCM10_Z1-1 | Tissue |
| 8 | 25346096 | T | C | CDCA2 | p.V521A | 0 | 67 | 0.409836 | 61 | WCM10_Z1-1 | Tissue |
| 8 | 27457417 | G | T | CLU | p.Y348* | 0 | 307 | 0.307692 | 260 | WCM10_Z1-1 | Tissue |
| 8 | 27462468 | G | A | CLU | p.Q268* | 0 | 93 | 0.397436 | 78 | WCM10_Z1-1 | Tissue |
| 8 | 30585270 | A | G | GSR | p.L28P | 0 | 35 | 0.317073 | 41 | WCM10_Z1-1 | Tissue |
| 8 | 37654892 | G | A | GPR124 | p.G36S | 0 | 49 | 0.404762 | 42 | WCM10_Z1-1 | Tissue |
| 8 | 39525646 | G | A | ADAM18 | p.G486R | 0 | 98 | 0.055046 | 109 | WCM10_Z1-1 | Tissue |
| 8 | 55533959 | G | A | RP1 | p.A145T | 0 | 214 | 0.359223 | 206 | WCM10_Z1-1 | Tissue |
| 8 | 623373 | C | T | ERICH1 | p.E327K | 0 | 356 | 0.341176 | 340 | WCM10_Z1-1 | Tissue |
| 8 | 72183995 | C | G | EYA1 | p.E322Q | 0 | 70 | 0.069444 | 72 | WCM10_Z1-1 | Tissue |
| 8 | 88249240 | C | T | CNBD1 | p.P224L | 0 | 97 | 0.050505 | 99 | WCM10_Z1-1 | Tissue |
| 8 | 95752008 | G | A | DPY19L4 | p.V201I | 0.005682 | 176 | 0.3625 | 160 | WCM10_Z1-1 | Tissue |
| 8 | 98289661 | C | T | TSPYL5 | p.V138I | 0 | 266 | 0.300926 | 216 | WCM10_Z1-1 | Tissue |
| 9 | 113169047 | T | C | SVEP1 | p.K2945E | 0 | 102 | 0.053763 | 93 | WCM10_Z1-1 | Tissue |
| 9 | 114840872 | G | A | SUSD1 | p.R567W | 0 | 116 | 0.318182 | 132 | WCM10_Z1-1 | Tissue |
| 9 | 114997111 | G | T | PTBP3 | p.P318H | 0 | 50 | 0.302326 | 43 | WCM10_Z1-1 | Tissue |
| 9 | 115336703 | C | T | KIAA1958 | p.R115W | 0 | 103 | 0.276786 | 112 | WCM10_Z1-1 | Tissue |
| 9 | 115408032 | G | A | KIAA1958 | p.R425Q | 0 | 210 | 0.05291 | 189 | WCM10_Z1-1 | Tissue |
| 9 | 130885298 | C | A | PTGES2 | p.G268C | 0 | 100 | 0.078431 | 102 | WCM10_Z1-1 | Tissue |
| 9 | 131388809 | C | T | SPTAN1 | p.A2135V | 0 | 86 | 0.306452 | 62 | WCM10_Z1-1 | Tissue |
| 9 | 134136426 | C | T | FAM78A | p.R212H | 0 | 263 | 0.271493 | 221 | WCM10_Z1-1 | Tissue |
| 9 | 134360347 | C | T | PRRC2B | p.R1860C | 0 | 150 | 0.278571 | 140 | WCM10_Z1-1 | Tissue |
| 9 | 135158791 | G | A | SETX | p.R2136C | 0 | 146 | 0.304762 | 105 | WCM10_Z1-1 | Tissue |
| 9 | 136559478 | G | A | SARDH | p.T608M | 0 | 47 | 0.096154 | 52 | WCM10_Z1-1 | Tissue |
| 9 | 137593102 | C | T | COL5A1 | p.R193C | 0 | 319 | 0.303371 | 267 | WCM10_Z1-1 | Tissue |
| 9 | 139101016 | C | T | QSOX2 | p.G552D | 0 | 67 | 0.5 | 50 | WCM10_Z1-1 | Tissue |
| 9 | 139272411 | G | A | SNAPC4 | p.R1290W | 0 | 35 | 0.270833 | 48 | WCM10_Z1-1 | Tissue |
| 9 | 139839861 | C | T | C8G | p.A30V | 0 | 150 | 0.342105 | 152 | WCM10_Z1-1 | Tissue |
| 9 | 139925634 | C | T | FUT7 | p.R186H | 0 | 70 | 0.385965 | 57 | WCM10_Z1-1 | Tissue |
| 9 | 140157579 | C | T | NELFB | p.R230W | 0 | 130 | 0.34188 | 117 | WCM10_Z1-1 | Tissue |
| 9 | 140852062 | C | T | CACNA1B | p.A419V | 0 | 44 | 0.25 | 36 | WCM10_Z1-1 | Tissue |
| 9 | 21859405 | A | G | MTAP | p.E282G | 0 | 44 | 0.26087 | 46 | WCM10_Z1-1 | Tissue |
| 9 | 33469279 | C | T | NOL6 | p.R263H | 0 | 220 | 0.290323 | 217 | WCM10_Z1-1 | Tissue |
| 9 | 35106881 | G | A | FAM214B | p.T301I | 0 | 83 | 0.095745 | 94 | WCM10_Z1-1 | Tissue |
| 9 | 35399709 | G | A | UNC13B | p.A1358T | 0 | 116 | 0.377049 | 122 | WCM10_Z1-1 | Tissue |
| 9 | 35558352 | G | T | RUSC2 | p.E1073D | 0 | 112 | 0.32 | 125 | WCM10_Z1-1 | Tissue |
| 9 | 35715064 | G | A | TLN1 | p.R916C | 0 | 489 | 0.332649 | 487 | WCM10_Z1-1 | Tissue |
| 9 | 71831325 | C | T | TJP2 | p.T62M | 0 | 54 | 0.311475 | 61 | WCM10_Z1-1 | Tissue |
| 9 | 91617077 | G | A | S1PR3 | p.R321Q | 0 | 146 | 0.316129 | 155 | WCM10_Z1-1 | Tissue |
| 9 | 99150565 | G | A | ZNF367 | p.A336V | 0 | 153 | 0.087591 | 137 | WCM10_Z1-1 | Tissue |
| X | 10174563 | C | A | CLCN4 | p.L241I | 0 | 155 | 0.137255 | 153 | WCM10_Z1-1 | Tissue |
| X | 103349130 | G | A | SLC25A53 | p.R271C | 0 | 49 | 0.611111 | 54 | WCM10_Z1-1 | Tissue |
| X | 109694200 | C | T | RGAG1 | p.P119S | 0 | 198 | 0.074713 | 174 | WCM10_Z1-1 | Tissue |
| X | 11682567 | G | A | ARHGAP6 | p.R128C | 0 | 57 | 0.106383 | 47 | WCM10_Z1-1 | Tissue |
| X | 142718456 | G | C | SLITRK4 | p.L157V | 0 | 80 | 0.628866 | 97 | WCM10_Z1-1 | Tissue |
| X | 153036318 | C | T | PLXNB3 | p.R706W | 0 | 52 | 0.431034 | 58 | WCM10_Z1-1 | Tissue |
| X | 153196242 | G | A | NAA10 | p.R149W | 0 | 39 | 0.594595 | 37 | WCM10_Z1-1 | Tissue |
| X | 153543623 | C | A | TKTL1 | p.S322Y | 0 | 78 | 0.063291 | 79 | WCM10_Z1-1 | Tissue |
| X | 24227134 | C | A | ZFX | p.P404H | 0 | 81 | 0.102564 | 78 | WCM10_Z1-1 | Tissue |
| X | 37586937 | G | A | XK | p.R186H | 0 | 76 | 0.717949 | 78 | WCM10_Z1-1 | Tissue |
| X | 48839662 | C | T | GRIPAP1 | p.R488H | 0 | 112 | 0.080808 | 99 | WCM10_Z1-1 | Tissue |
| X | 49035644 | C | T | PRICKLE3 | p.V174M | 0 | 81 | 0.629032 | 62 | WCM10_Z1-1 | Tissue |
| X | 53970674 | G | A | PHF8 | p.R884C | 0 | 104 | 0.681159 | 138 | WCM10_Z1-1 | Tissue |
| X | 69478490 | G | A | P2RY4 | p.R329C | 0 | 114 | 0.65625 | 96 | WCM10_Z1-1 | Tissue |
| X | 70643034 | C | T | TAF1 | p.A1506V | 0 | 77 | 0.616667 | 60 | WCM10_Z1-1 | Tissue |
| X | 75649389 | G | A | MAGEE1 | p.V356I | 0 | 157 | 0.694118 | 170 | WCM10_Z1-1 | Tissue |
| 11 | 19170770 | G | T | ZDHHC13 | p.Q137H | 0 | 39 | 0.357143 | 98 | WCM119 | Plasma |
| 1 | 232568026 | G | A | SIPA1L2 | p.P1408L | 0 | 50 | 0.255319 | 94 | WCM119 | Plasma |
| 12 | 75601178 | C | G | KCNC2 | p.A196P | 0 | 33 | 0.337662 | 77 | WCM119 | Plasma |
| 17 | 17699855 | G | A | RAI1 | p.R1198K | 0 | 41 | 0.096774 | 62 | WCM119 | Plasma |
| 17 | 38510644 | G | A | RARA | p.A300T | 0 | 37 | 0.4 | 55 | WCM119 | Plasma |
| 17 | 60040181 | G | C | MED13 | p.L1666V | 0 | 47 | 0.211765 | 85 | WCM119 | Plasma |
| 18 | 77896514 | T | C | ADNP2 | p.I1073T | 0 | 56 | 0.442623 | 61 | WCM119 | Plasma |
| 20 | 47569385 | G | A | AREGEF2 | p.M189I | 0 | 38 | 0.488095 | 84 | WCM119 | Plasma |
| 2 | 183088628 | G | T | PDE1A | p.H263N | 0 | 56 | 0.307692 | 91 | WCM119 | Plasma |
| 22 | 20800753 | C | T | KLHL22 | p.G506R | 0 | 38 | 0.366337 | 101 | WCM119 | Plasma |
| 3 | 148756856 | G | C | HLTF | p.R926G | 0 | 57 | 0.326316 | 95 | WCM119 | Plasma |
| 3 | 56675432 | G | C | FAM208A | p.S855C | 0 | 61 | 0.348624 | 109 | WCM119 | Plasma |
| 8 | 4494994 | A | T | CSMD1 | p.C58S | 0 | 45 | 0.148649 | 74 | WCM119 | Plasma |
| 9 | 113243636 | A | T | SVEP1 | p.L751I | 0 | 52 | 0.310345 | 87 | WCM119 | Plasma |
| 10 | 17905650 | G | A | MRC1L1 | p.E581K | 0 | 34 | 0.540541 | 37 | WCM119_Z6_1 | Tissue |
| 11 | 8111755 | G | T | TUB | p.S77I | 0 | 50 | 0.051724 | 58 | WCM119_Z6_1 | Tissue |
| 1 | 203134607 | T | C | ADORA1 | p.V187A | 0 | 46 | 0.150943 | 53 | WCM119_Z6_1 | Tissue |
| 12 | 57485590 | T | C | NAB2 | p.F256L | 0 | 73 | 0.246914 | 81 | WCM119_Z6_1 | Tissue |
| 17 | 38279626 | G | T | MSL1 | p.R254I | 0 | 142 | 0.594828 | 232 | WCM119_Z6_1 | Tissue |
| 17 | 60040181 | G | C | MED13 | p.L1666V | 0 | 56 | 0.208333 | 48 | WCM119_Z6_1 | Tissue |
| 19 | 55447768 | G | A | NLRP7 | p.R721W | 0 | 55 | 0.164384 | 73 | WCM119_Z6_1 | Tissue |
| 1 | 95699815 | C | T | RWDD3 | p.S10L | 0 | 41 | 0.219512 | 41 | WCM119_Z6_1 | Tissue |
| 19 | 6222644 | C | T | MLLT1 | p.E200K | 0 | 109 | 0.246269 | 134 | WCM119_Z6_1 | Tissue |
| 21 | 43166878 | C | T | RIPK4 | p.E243K | 0 | 149 | 0.083333 | 132 | WCM119_Z6_1 | Tissue |
| 22 | 42089894 | G | A | C22orf46 | p.R215H | 0 | 72 | 0.057692 | 52 | WCM119_Z6_1 | Tissue |
| 3 | 121353250 | C | T | HCLS1 | p.R236H | 0 | 64 | 0.269231 | 52 | WCM119_Z6_1 | Tissue |
| 3 | 130116596 | G | C | COL6A5 | p.L1246F | 0 | 116 | 0.064748 | 139 | WCM119_Z6_1 | Tissue |
| 3 | 148756856 | G | C | HLTF | p.R926G | 0 | 117 | 0.326531 | 147 | WCM119_Z6_1 | Tissue |
| 3 | 56675432 | G | C | FAM208A | p.S855C | 0 | 99 | 0.363636 | 88 | WCM119_Z6_1 | Tissue |
| 5 | 124080030 | A | C | ZNF608 | p.L218R | 0 | 32 | 0.5 | 34 | WCM119_Z6_1 | Tissue |
| 6 | 155154029 | G | A | SCAF8 | p.V1106I | 0 | 83 | 0.109756 | 82 | WCM119_Z6_1 | Tissue |
| 7 | 117431424 | G | A | CTTNBP2 | p.P609L | 0 | 173 | 0.259459 | 185 | WCM119_Z6_1 | Tissue |
| 7 | 15725552 | G | A | MEOX2 | p.A159V | 0 | 56 | 0.190476 | 42 | WCM119_Z6_1 | Tissue |
| 7 | 54617755 | G | A | VSTM2A | p.V176I | 0 | 106 | 0.502674 | 187 | WCM119_Z6_1 | Tissue |
| 8 | 113420582 | G | T | CSMD3 | p.P1857Q | 0 | 59 | 0.322917 | 96 | WCM119_Z6_1 | Tissue |
| 8 | 1497307 | C | T | DLGAP2 | p.R150C | 0 | 99 | 0.206186 | 97 | WCM119_Z6_1 | Tissue |
| 8 | 4494994 | A | T | CSMD1 | p.C58S | 0 | 69 | 0.144144 | 111 | WCM119_Z6_1 | Tissue |
| 15 | 43783909 | C | G | TP53BP1 | p.S105T | 0 | 54 | 0.075949 | 79 | WCM121 | Plasma |
| 15 | 43783910 | T | C | TP53BP1 | p.S105G | 0 | 54 | 0.076923 | 78 | WCM121 | Plasma |
| 1 | 144918897 | C | T | PDE4DIP | p.R430Q | 0 | 45 | 0.111111 | 36 | WCM121_Z1_1 | Tissue |
| 12 | 122252481 | C | T | SETD1B | p.T787M | 0 | 103 | 0.273684 | 95 | WCM121_Z1_1 | Tissue |
| 12 | 22013982 | C | A | ABCC9 | p.A783S | 0 | 42 | 0.202703 | 74 | WCM121_Z1_1 | Tissue |
| 1 | 235972899 | C | A | LYST | p.G407* | 0 | 206 | 0.184211 | 190 | WCM121_Z1_1 | Tissue |
| 13 | 21306151 | C | T | N6AMT2 | p.E113K | 0 | 111 | 0.109589 | 73 | WCM121_Z1_1 | Tissue |
| 13 | 24876763 | C | T | SPATA13 | p.P604L | 0 | 141 | 0.117188 | 128 | WCM121_Z1_1 | Tissue |
| 1 | 32797822 | A | G | HDAC1 | p.K451E | 0 | 43 | 0.220588 | 68 | WCM121_Z1_1 | Tissue |
| 15 | 74290754 | G | T | PML | p.G180V | 0 | 85 | 0.380952 | 63 | WCM121_Z1_1 | Tissue |
| 19 | 39667297 | T | G | PAK4 | p.V476G | 0 | 125 | 0.223684 | 152 | WCM121_Z1_1 | Tissue |
| 19 | 40520343 | A | T | ZNF546 | p.E389V | 0 | 66 | 0.168831 | 77 | WCM121_Z1_1 | Tissue |
| 2 | 128380840 | G | A | MYO7B | p.V1211I | 0 | 111 | 0.135922 | 103 | WCM121_Z1_1 | Tissue |
| 21 | 47969722 | G | A | DIP2A | p.R854Q | 0 | 203 | 0.138776 | 245 | WCM121_Z1_1 | Tissue |
| 2 | 234738282 | G | A | MROH2A | p.R1488H | 0 | 57 | 0.170455 | 88 | WCM121_Z1_1 | Tissue |
| 2 | 65299398 | G | C | CEP68 | p.G390R | 0 | 227 | 0.5 | 182 | WCM121_Z1_1 | Tissue |
| 3 | 197670800 | T | C | IQCG | p.E44G | 0 | 88 | 0.394231 | 104 | WCM121_Z1_1 | Tissue |
| 3 | 39230287 | A | G | XIRP1 | p.L217P | 0 | 213 | 0.156951 | 223 | WCM121_Z1_1 | Tissue |
| 3 | 52883823 | G | A | TMEM110 | p.R138C | 0 | 123 | 0.189655 | 116 | WCM121_Z1_1 | Tissue |
| 4 | 39448474 | G | C | KLB | p.G710R | 0 | 46 | 0.264151 | 53 | WCM121_Z1_1 | Tissue |
| 4 | 84374518 | G | C | HELQ | p.T293S | 0 | 98 | 0.342593 | 108 | WCM121_Z1_1 | Tissue |
| 9 | 133961040 | C | A | LAMC3 | p.A1387E | 0 | 72 | 0.081081 | 74 | WCM121_Z1_1 | Tissue |
| 9 | 26923260 | G | A | PLAA | p.H319Y | 0 | 36 | 0.159091 | 44 | WCM121_Z1_1 | Tissue |
| 9 | 36110059 | A | C | RECK | p.D624A | 0 | 191 | 0.14359 | 195 | WCM121_Z1_1 | Tissue |
| 9 | 84605740 | C | A | SPATA31D1 | p.H119N | 0 | 57 | 0.128205 | 39 | WCM121_Z1_1 | Tissue |
| 10 | 27702774 | C | T | PTCHD3 | p.A136T | 0 | 66 | 0.368421 | 57 | WCM124_X1_3 | Tissue |
| 10 | 7285660 | T | C | SFMBT2 | p.N327S | 0 | 37 | 0.258065 | 31 | WCM124_X1_3 | Tissue |
| 11 | 6478162 | G | A | TRIMS | p.P265L | 0 | 122 | 0.490066 | 151 | WCM124_X1_3 | Tissue |
| 11 | 76751319 | G | A | B3GNT6 | p.G242S | 0 | 50 | 0.082192 | 73 | WCM124_X1_3 | Tissue |
| 1 | 186043891 | C | T | HMCN1 | p.H2720Y | 0 | 37 | 0.220339 | 59 | WCM124_X1_3 | Tissue |
| 11 | 87882847 | G | T | RAB38 | p.A160E | 0 | 227 | 0.067511 | 237 | WCM124_X1_3 | Tissue |
| 1 | 215821900 | G | T | USH2A | p.P4851H | 0 | 71 | 0.193548 | 124 | WCM124_X1_3 | Tissue |
| 12 | 52467672 | G | T | C12orf44 | p.V80F | 0 | 103 | 0.090164 | 122 | WCM124_X1_3 | Tissue |
| 1 | 27690551 | G | A | MAP3K6 | p.R241W | 0 | 67 | 0.256757 | 74 | WCM124_X1_3 | Tissue |
| 13 | 27333336 | A | C | GPR12 | p.F210C | 0 | 106 | 0.405882 | 170 | WCM124_X1_3 | Tissue |
| 14 | 96783585 | C | T | ATG2B | p.R1036H | 0 | 100 | 0.275 | 80 | WCM124_X1_3 | Tissue |
| 1 | 53793511 | T | C | LRP8 | p.Q25R | 0 | 42 | 0.083333 | 48 | WCM124_X1_3 | Tissue |
| 15 | 85189436 | G | A | WDR73 | p.R166W | 0 | 155 | 0.433071 | 127 | WCM124_X1_3 | Tissue |
| 16 | 57496641 | C | T | POLR2C | p.P2L | 0 | 42 | 0.424242 | 33 | WCM124_X1_3 | Tissue |
| 17 | 27064987 | T | G | NEKS | p.V347G | 0 | 51 | 0.166667 | 66 | WCM124_X1_3 | Tissue |
| 17 | 37922387 | G | A | IKZF3 | p.R396C | 0 | 163 | 0.108808 | 193 | WCM124_X1_3 | Tissue |
| 17 | 40065910 | T | C | ACLY | p.D130G | 0 | 189 | 0.108974 | 156 | WCM124_X1_3 | Tissue |
| 17 | 71197854 | C | T | COG1 | p.Q630* | 0 | 124 | 0.26875 | 160 | WCM124_X1_3 | Tissue |
| 19 | 45656197 | C | T | NKPD1 | p.A278T | 0 | 33 | 0.444444 | 36 | WCM124_X1_3 | Tissue |
| 19 | 6230663 | G | C | MLLT1 | p.P113R | 0 | 34 | 0.36 | 100 | WCM124_X1_3 | Tissue |
| 2 | 111395672 | G | T | BUB1 | p.H1043N | 0 | 39 | 0.142857 | 42 | WCM124_X1_3 | Tissue |
| 5 | 141334958 | G | A | PCDH12 | p.P820L | 0 | 157 | 0.384615 | 130 | WCM124_X1_3 | Tissue |
| 6 | 159636090 | C | T | FNDC1 | p.R192W | 0 | 71 | 0.352113 | 71 | WCM124_X1_3 | Tissue |
| 6 | 97001230 | A | C | UFL1 | p.T746P | 0 | 48 | 0.382979 | 47 | WCM124_X1_3 | Tissue |
| 7 | 106509446 | C | A | PIK3CG | p.Y480* | 0 | 101 | 0.338462 | 65 | WCM124_X1_3 | Tissue |
| 9 | 129458674 | G | A | LMX1B | p.V385M | 0 | 176 | 0.154839 | 310 | WCM124_X1_3 | Tissue |
| 10 | 95326847 | G | T | FFAR4 | p.V124F | 0 | 196 | 0.738889 | 180 | PM12_Z10_1 | Tissue |
| 11 | 12029948 | G | A | DKK3 | p.T61M | 0 | 75 | 0.131579 | 38 | PM12_Z10_1 | Tissue |
| 11 | 17546056 | G | T | USH1C | p.P234H | 0 | 139 | 0.06701 | 194 | PM12_Z10_1 | Tissue |
| 1 | 118535220 | T | C | SPAG17 | p.I1744V | 0 | 263 | 0.126354 | 277 | PM12_Z10_1 | Tissue |
| 1 | 118579497 | T | A | SPAG17 | p.D1110V | 0 | 61 | 0.126582 | 79 | PM12_Z10_1 | Tissue |
| 1 | 158047834 | G | A | KIRREL | p.E86K | 0 | 203 | 0.268012 | 347 | PM12_Z10_1 | Tissue |
| 11 | 71944012 | C | T | INPPL1 | p.R649* | 0 | 164 | 0.052083 | 96 | PM12_Z10_1 | Tissue |
| 1 | 175957441 | C | G | RFWD2 | p.G652A | 0 | 79 | 0.157658 | 222 | PM12_Z10_1 | Tissue |
| 1 | 204948171 | G | C | NFASC | p.G651A | 0 | 103 | 0.06414 | 343 | PM12_Z10_1 | Tissue |
| 12 | 123459815 | C | T | OGFOD2 | p.A6V | 0 | 70 | 0.487179 | 39 | PM12_Z10_1 | Tissue |
| 1 | 227077810 | A | T | PSEN2 | p.I288L | 0 | 140 | 0.192893 | 197 | PM12_Z10_1 | Tissue |
| 1 | 228482627 | C | G | OBSCN | p.L3848V | 0.002342 | 427 | 0.358607 | 488 | PM12_Z10_1 | Tissue |
| 12 | 42717823 | G | C | ZCRB1 | p.R28G | 0 | 110 | 0.191011 | 89 | PM12_Z10_1 | Tissue |
| 13 | 25281512 | G | A | ATP12A | p.G812R | 0 | 44 | 0.073171 | 41 | PM12_Z10_1 | Tissue |
| 15 | 31619458 | C | G | KLF13 | p.L15V | 0 | 74 | 0.322222 | 90 | PM12_Z10_1 | Tissue |
| 1 | 53236935 | C | G | ZYG11B | p.T147S | 0 | 55 | 0.307692 | 39 | PM12_Z10_1 | Tissue |
| 1 | 53323317 | G | A | ZYG11A | p.A302T | 0 | 179 | 0.085227 | 176 | PM12_Z10_1 | Tissue |
| 1 | 62738884 | G | T | KANK4 | p.P631Q | 0 | 62 | 0.0625 | 48 | PM12_Z10_1 | Tissue |
| 16 | 30777502 | G | T | RNF40 | p.E338* | 0 | 155 | 0.141553 | 219 | PM12_Z10_1 | Tissue |
| 17 | 2323445 | C | G | METTL16 | p.R503T | 0 | 149 | 0.285714 | 98 | PM12_Z10_1 | Tissue |
| 17 | 76993090 | G | T | CANT1 | p.D205E | 0 | 255 | 0.11215 | 214 | PM12_Z10_1 | Tissue |
| 19 | 35829124 | A | T | CD22 | p.S347C | 0 | 216 | 0.116071 | 224 | PM12_Z10_1 | Tissue |
| 19 | 38704297 | G | A | DPF1 | p.T316I | 0 | 117 | 0.149254 | 67 | PM12_Z10_1 | Tissue |
| 20 | 40052263 | G | T | CHD6 | p.T1475N | 0 | 40 | 0.462963 | 54 | PM12_Z10_1 | Tissue |
| 20 | 44997585 | G | A | ELMO2 | p.S636F | 0 | 121 | 0.078125 | 128 | PM12_Z10_1 | Tissue |
| 2 | 108924898 | T | C | SULT1C2 | p.I304T | 0 | 131 | 0.159292 | 113 | PM12_Z10_1 | Tissue |
| 2 | 113479447 | G | A | NT5DC4 | p.D22N | 0 | 91 | 0.343284 | 67 | PM12_Z10_1 | Tissue |
| 2 | 149854985 | A | C | KIF5C | p.E724D | 0 | 150 | 0.103448 | 145 | PM12_Z10_1 | Tissue |
| 2 | 152406251 | G | C | NEB | p.L4949V | 0 | 131 | 0.513369 | 187 | PM12_Z10_1 | Tissue |
| 2 | 15608610 | T | A | NBAS | p.R591S | 0 | 157 | 0.429577 | 142 | PM12_Z10_1 | Tissue |
| 2 | 190315633 | A | C | WDR75 | p.Y74S | 0 | 61 | 0.152 | 125 | PM12_Z10_1 | Tissue |
| 2 | 209138323 | G | T | PIKFYVE | p.G63V | 0 | 166 | 0.055556 | 234 | PM12_Z10_1 | Tissue |
| 2 | 239257426 | G | A | TRAF3IP1 | p.G435D | 0 | 77 | 0.107692 | 65 | PM12_Z10_1 | Tissue |
| 2 | 242683147 | G | A | D2HGDH | p.G201R | 0 | 140 | 0.0625 | 80 | PM12_Z10_1 | Tissue |
| 2 | 286134 | G | T | FAM150B | p.T126N | 0 | 52 | 0.450549 | 91 | PM12_Z10_1 | Tissue |
| 2 | 9083337 | A | T | MBOAT2 | p.I93K | 0 | 44 | 0.35 | 40 | PM12_Z10_1 | Tissue |
| 3 | 122631875 | G | T | SEMA5B | p.T847N | 0 | 40 | 0.54717 | 53 | PM12_Z10_1 | Tissue |
| 3 | 165547604 | A | T | BCHE | p.D406E | 0 | 138 | 0.071942 | 139 | PM12_Z10_1 | Tissue |
| 3 | 69242846 | C | T | FRMD4B | p.R556Q | 0 | 83 | 0.090909 | 66 | PM12_Z10_1 | Tissue |
| 4 | 109663687 | T | C | ETNPPL | p.M485V | 0 | 111 | 0.377953 | 127 | PM12_Z10_1 | Tissue |
| 4 | 68930433 | C | T | TMPRSS11F | p.V329I | 0 | 53 | 0.050955 | 157 | PM12_Z10_1 | Tissue |
| 5 | 121487959 | C | T | ZNF474 | p.P92S | 0 | 148 | 0.081818 | 110 | PM12_Z10_1 | Tissue |
| 5 | 1244730 | G | A | SLC6A18 | p.D502N | 0 | 149 | 0.64557 | 79 | PM12_Z10_1 | Tissue |
| 5 | 140475710 | G | A | PCDHB2 | p.V446I | 0 | 135 | 0.130081 | 123 | PM12_Z10_1 | Tissue |
| 5 | 140857128 | G | A | PCDHGC3 | p.W482* | 0 | 86 | 0.060606 | 66 | PM12_Z10_1 | Tissue |
| 5 | 166711927 | A | G | TENM2 | p.S29G | 0 | 67 | 0.1625 | 80 | PM12_Z10_1 | Tissue |
| 5 | 53815095 | T | C | SNX18 | p.M438T | 0 | 305 | 0.377581 | 339 | PM12_Z10_1 | Tissue |
| 6 | 90402844 | C | T | MDN1 | p.R3302Q | 0 | 35 | 0.06383 | 47 | PM12_Z10_1 | Tissue |
| 7 | 151873569 | T | G | KMT2C | p.N2990T | 0 | 237 | 0.051672 | 329 | PM12_Z10_1 | Tissue |
| 7 | 36338762 | G | A | EEPD1 | p.G553S | 0 | 56 | 0.068182 | 44 | PM12_Z10_1 | Tissue |
| 7 | 94989397 | G | A | PON3 | p.T318I | 0 | 49 | 0.089552 | 67 | PM12_Z10_1 | Tissue |
| 8 | 142367144 | G | C | GPR20 | p.P294A | 0 | 147 | 0.262948 | 251 | PM12_Z10_1 | Tissue |
| 9 | 113192211 | T | G | SVEP1 | p.K1868N | 0 | 92 | 0.414286 | 140 | PM12_Z10_1 | Tissue |
| 9 | 13206032 | G | T | MPDZ | p.Q453K | 0 | 53 | 0.107143 | 56 | PM12_Z10_1 | Tissue |
| 9 | 139311463 | A | C | PMPCA | p.K232Q | 0 | 131 | 0.358974 | 156 | PM12_Z10_1 | Tissue |
| X | 16170596 | G | C | GRPR | p.S328T | 0 | 93 | 0.066667 | 120 | PM12_Z10_1 | Tissue |
| 10 | 124380665 | G | A | DMBT1 | p.V1664M | 0 | 89 | 0.14433 | 97 | WCM131 | Plasma |
| 10 | 13325796 | T | C | PHYH | p.E241G | 0 | 79 | 0.117647 | 85 | WCM131 | Plasma |
| 10 | 134750473 | C | T | TTC40 | p.M249I | 0 | 41 | 0.133333 | 60 | WCM131 | Plasma |
| 10 | 134895273 | G | A | GPR123 | p.V306M | 0 | 42 | 0.176471 | 68 | WCM131 | Plasma |
| 10 | 25465161 | A | G | GPR158 | p.E271G | 0 | 36 | 0.132075 | 53 | WCM131 | Plasma |
| 10 | 50025396 | G | A | WDFY4 | p.R1816Q | 0 | 42 | 0.126984 | 63 | WCM131 | Plasma |
| 10 | 5498098 | G | A | NET1 | p.V416I | 0 | 49 | 0.179104 | 67 | WCM131 | Plasma |
| 10 | 82012967 | G | T | AL359195.1 | p.C162F | 0 | 33 | 0.08 | 50 | WCM131 | Plasma |
| 10 | 82013119 | G | A | AL359195.1 | p.G213S | 0 | 45 | 0.210526 | 76 | WCM131 | Plasma |
| 10 | 82187167 | G | A | FAM213A | p.R164Q | 0 | 72 | 0.163462 | 104 | WCM131 | Plasma |
| 11 | 100820663 | G | A | ARHGAP42 | p.A380T | 0 | 70 | 0.076923 | 91 | WCM131 | Plasma |
| 11 | 103006228 | T | A | DYNC2H1 | p.F737Y | 0 | 46 | 0.095238 | 63 | WCM131 | Plasma |
| 11 | 104912206 | G | A | CARD16 | p.P172L | 0 | 60 | 0.150685 | 73 | WCM131 | Plasma |
| 11 | 113639557 | C | T | ZW10 | p.E80K | 0 | 56 | 0.220588 | 68 | WCM131 | Plasma |
| 11 | 11374269 | A | G | CSNK2A3 | p.I133T | 0 | 62 | 0.15625 | 96 | WCM131 | Plasma |
| 11 | 114401611 | A | G | NXPE1 | p.L40S | 0 | 31 | 0.1 | 60 | WCM131 | Plasma |
| 11 | 114550423 | T | G | NXPE2 | p.L24R | 0 | 56 | 0.075 | 80 | WCM131 | Plasma |
| 11 | 114568942 | T | C | NXPE2 | p.V103A | 0 | 57 | 0.197183 | 71 | WCM131 | Plasma |
| 11 | 118362527 | C | T | KMT2A | p.R1627W | 0 | 45 | 0.178571 | 56 | WCM131 | Plasma |
| 1 | 111998809 | A | G | ATP5F1 | p.M109V | 0 | 33 | 0.166667 | 60 | WCM131 | Plasma |
| 11 | 120041110 | T | A | AP000679.2 | p.L198Q | 0 | 36 | 0.12 | 50 | WCM131 | Plasma |
| 11 | 123676829 | G | C | OR6M1 | p.P77A | 0 | 45 | 0.133333 | 90 | WCM131 | Plasma |
| 11 | 124056566 | G | A | OR10D3 | p.R197K | 0 | 35 | 0.15873 | 63 | WCM131 | Plasma |
| 11 | 124056732 | T | G | OR10D3 | p.Y252* | 0 | 42 | 0.135593 | 59 | WCM131 | Plasma |
| 11 | 124056787 | G | A | OR10D3 | p.V271M | 0 | 35 | 0.189655 | 58 | WCM131 | Plasma |
| 1 | 113265772 | C | T | FAM19A3 | p.P84S | 0 | 33 | 0.075472 | 53 | WCM131 | Plasma |
| 1 | 117619377 | C | G | TTF2 | p.P502R | 0 | 35 | 0.197183 | 71 | WCM131 | Plasma |
| 11 | 17629855 | C | T | OTOG | p.S1402L | 0 | 37 | 0.076923 | 65 | WCM131 | Plasma |
| 11 | 43428757 | A | G | TTC17 | p.I611V | 0 | 31 | 0.212766 | 47 | WCM131 | Plasma |
| 11 | 48387186 | A | G | OR4C5 | p.F278L | 0 | 72 | 0.094118 | 85 | WCM131 | Plasma |
| 11 | 49974261 | T | C | OR4C13 | p.M96T | 0 | 51 | 0.138889 | 72 | WCM131 | Plasma |
| 1 | 151638469 | G | A | SNX27 | p.V323M | 0 | 70 | 0.139535 | 86 | WCM131 | Plasma |
| 1 | 160917793 | G | A | ITLN2 | p.R251W | 0 | 51 | 0.151899 | 79 | WCM131 | Plasma |
| 11 | 62863518 | A | G | SLC22A24 | p.S339P | 0 | 32 | 0.104478 | 67 | WCM131 | Plasma |
| 11 | 66358007 | C | T | CCDC87 | p.R827H | 0 | 37 | 0.090909 | 55 | WCM131 | Plasma |
| 1 | 171118840 | G | A | FMO6P | p.V257I | 0 | 59 | 0.055556 | 72 | WCM131 | Plasma |
| 11 | 73020965 | C | A | ARHGEF17 | p.L428M | 0 | 31 | 0.134615 | 52 | WCM131 | Plasma |
| 1 | 173873176 | C | A | SERPINC1 | p.A416S | 0 | 43 | 0.089286 | 56 | WCM131 | Plasma |
| 11 | 74203308 | T | A | LIPT2 | p.T190S | 0 | 43 | 0.112903 | 62 | WCM131 | Plasma |
| 11 | 75439104 | C | T | MOGAT2 | p.Q189* | 0 | 31 | 0.173333 | 75 | WCM131 | Plasma |
| 11 | 7818410 | C | T | OR5P2 | p.R27Q | 0 | 38 | 0.125 | 80 | WCM131 | Plasma |
| 11 | 92088067 | A | G | FAT3 | p.N930S | 0 | 53 | 0.180556 | 72 | WCM131 | Plasma |
| 11 | 93457532 | C | G | KIAA1731 | p.D2103E | 0 | 36 | 0.183333 | 60 | WCM131 | Plasma |
| 11 | 93458848 | C | G | KIAA1731 | p.A2117G | 0 | 41 | 0.134328 | 67 | WCM131 | Plasma |
| 1 | 20640926 | T | C | VWA5B1 | p.I135T | 0 | 33 | 0.116667 | 60 | WCM131 | Plasma |
| 12 | 101016068 | G | A | GAS2L3 | p.E222K | 0 | 61 | 0.132353 | 68 | WCM131 | Plasma |
| 12 | 101016071 | G | A | GAS2L3 | p.D223N | 0 | 61 | 0.136364 | 66 | WCM131 | Plasma |
| 12 | 113384741 | A | G | OAS3 | p.D277G | 0 | 43 | 0.168831 | 77 | WCM131 | Plasma |
| 12 | 129153986 | G | A | TMEM132C | p.V444I | 0 | 54 | 0.202247 | 89 | WCM131 | Plasma |
| 1 | 212988440 | T | A | TATDN3 | p.V256E | 0 | 36 | 0.101695 | 59 | WCM131 | Plasma |
| 12 | 13061554 | G | A | GPRC5A | p.R124Q | 0 | 42 | 0.195122 | 82 | WCM131 | Plasma |
| 12 | 130923011 | C | T | RIMBP2 | p.A502T | 0 | 40 | 0.139535 | 43 | WCM131 | Plasma |
| 12 | 132401575 | C | T | ULK1 | p.T717M | 0 | 58 | 0.157895 | 76 | WCM131 | Plasma |
| 1 | 225565015 | T | C | DNAH14 | p.L3088P | 0 | 31 | 0.298246 | 57 | WCM131 | Plasma |
| 1 | 225569241 | T | G | DNAH14 | p.F3236C | 0 | 58 | 0.242857 | 70 | WCM131 | Plasma |
| 12 | 40821867 | G | A | MUC19 | p.V206I | 0 | 50 | 0.193182 | 88 | WCM131 | Plasma |
| 12 | 40832161 | T | C | MUC19 | p.I567T | 0 | 49 | 0.230769 | 65 | WCM131 | Plasma |
| 12 | 40835734 | C | G | MUC19 | p.I694M | 0 | 61 | 0.149425 | 87 | WCM131 | Plasma |
| 12 | 40838216 | T | C | MUC19 | p.V909A | 0 | 45 | 0.189655 | 58 | WCM131 | Plasma |
| 12 | 50744119 | G | A | FAM186A | p.H2166Y | 0 | 32 | 0.0625 | 64 | WCM131 | Plasma |
| 12 | 50754563 | T | G | FAM186A | p.K187Q | 0 | 36 | 0.095238 | 63 | WCM131 | Plasma |
| 12 | 53162813 | C | A | KRT76 | p.S534I | 0 | 36 | 0.20339 | 59 | WCM131 | Plasma |
| 12 | 7252566 | C | T | C1RL | p.A183T | 0 | 33 | 0.173913 | 46 | WCM131 | Plasma |
| 12 | 80899901 | C | A | PTPRQ | p.Q787K | 0 | 53 | 0.095238 | 84 | WCM131 | Plasma |
| 1 | 28208868 | C | T | THEMIS2 | p.R345W | 0 | 49 | 0.195402 | 87 | WCM131 | Plasma |
| 12 | 96934699 | A | G | C12orf55 | p.T671A | 0 | 43 | 0.148148 | 54 | WCM131 | Plasma |
| 12 | 97023939 | A | T | C12orf55 | p.N1391I | 0 | 55 | 0.1125 | 80 | WCM131 | Plasma |
| 12 | 97024355 | A | C | C12orf55 | p.R1414S | 0 | 62 | 0.162791 | 86 | WCM131 | Plasma |
| 12 | 97186242 | C | A | C12orf55 | p.P2939T | 0 | 63 | 0.157303 | 89 | WCM131 | Plasma |
| 13 | 103384712 | G | A | CCDC168 | p.T1483M | 0 | 56 | 0.179775 | 89 | WCM131 | Plasma |
| 13 | 112278413 | C | T | RP11-65D24.2 | p.P24L | 0 | 37 | 0.142857 | 63 | WCM131 | Plasma |
| 13 | 30829740 | C | G | KATNAL1 | p.Q112H | 0 | 35 | 0.085106 | 47 | WCM131 | Plasma |
| 13 | 32799081 | C | T | FRY | p.A1643V | 0 | 46 | 0.1 | 70 | WCM131 | Plasma |
| 13 | 36886481 | C | T | SPG20 | p.M539I | 0 | 51 | 0.111111 | 81 | WCM131 | Plasma |
| 13 | 60485919 | A | G | DIAPH3 | p.F773L | 0 | 31 | 0.213115 | 61 | WCM131 | Plasma |
| 13 | 99462473 | C | T | DOCK9 | p.D1735N | 0 | 37 | 0.14 | 50 | WCM131 | Plasma |
| 14 | 100795151 | C | T | SLC25A47 | p.S139L | 0 | 31 | 0.108108 | 37 | WCM131 | Plasma |
| 14 | 21498871 | C | G | TPPP2 | p.T44R | 0 | 32 | 0.142857 | 49 | WCM131 | Plasma |
| 14 | 23102882 | C | A | OR6J1 | p.V279L | 0 | 31 | 0.075472 | 53 | WCM131 | Plasma |
| 14 | 51368610 | A | G | ABHD12B | p.I282V | 0 | 68 | 0.15942 | 69 | WCM131 | Plasma |
| 14 | 59988324 | C | T | CCDC175 | p.S689N | 0 | 54 | 0.183333 | 60 | WCM131 | Plasma |
| 1 | 49119120 | A | G | AGBL4 | p.I243T | 0 | 40 | 0.2 | 65 | WCM131 | Plasma |
| 15 | 23609550 | G | T | GOLGA8S | p.E470* | 0 | 55 | 0.159091 | 44 | WCM131 | Plasma |
| 1 | 52747414 | G | A | ZFYVE9 | p.C984Y | 0 | 33 | 0.147541 | 61 | WCM131 | Plasma |
| 1 | 53329820 | T | G | ZYG11A | p.H439Q | 0 | 43 | 0.105263 | 57 | WCM131 | Plasma |
| 15 | 33445701 | A | G | FMN1 | p.L472P | 0 | 31 | 0.177419 | 62 | WCM131 | Plasma |
| 15 | 33446947 | A | C | FMN1 | p.S57A | 0 | 38 | 0.114286 | 70 | WCM131 | Plasma |
| 15 | 42342824 | T | G | PLA2G4E | p.E26D | 0 | 53 | 0.073529 | 68 | WCM131 | Plasma |
| 15 | 43783909 | C | G | TP53BP1 | p.S105T | 0 | 50 | 0.087719 | 57 | WCM131 | Plasma |
| 15 | 43783910 | T | C | TP53BP1 | p.S105G | 0 | 49 | 0.089286 | 56 | WCM131 | Plasma |
| 1 | 57349308 | C | T | C8A | p.S270L | 0 | 44 | 0.118421 | 76 | WCM131 | Plasma |
| 15 | 81214459 | G | A | KIAA1199 | p.R728Q | 0 | 53 | 0.098765 | 81 | WCM131 | Plasma |
| 16 | 14345779 | G | A | MKL2 | p.A811T | 0 | 33 | 0.137931 | 58 | WCM131 | Plasma |
| 16 | 1484528 | G | A | CCDC154 | p.R636C | 0 | 31 | 0.111111 | 72 | WCM131 | Plasma |
| 16 | 2161666 | G | A | PKD1 | p.P1168S | 0 | 51 | 0.148649 | 74 | WCM131 | Plasma |
| 16 | 30772988 | C | T | C16orf93 | p.A28T | 0 | 56 | 0.152542 | 59 | WCM131 | Plasma |
| 16 | 4787917 | G | T | C16orf71 | p.W82C | 0 | 31 | 0.146341 | 41 | WCM131 | Plasma |
| 16 | 67314790 | C | T | PLEKHG4 | p.R201W | 0 | 31 | 0.15 | 60 | WCM131 | Plasma |
| 16 | 80584435 | A | C | DYNLRB2 | p.I91L | 0 | 44 | 0.118421 | 76 | WCM131 | Plasma |
| 16 | 85144059 | G | C | FAM92B | p.Q10E | 0 | 37 | 0.21875 | 64 | WCM131 | Plasma |
| 17 | 18881605 | C | A | FAM83G | p.Q458H | 0 | 45 | 0.145161 | 62 | WCM131 | Plasma |
| 17 | 26822793 | G | A | SLC13A2 | p.V477M | 0 | 33 | 0.203125 | 64 | WCM131 | Plasma |
| 17 | 39538524 | C | T | KRT34 | p.C34Y | 0 | 36 | 0.114286 | 70 | WCM131 | Plasma |
| 17 | 4058094 | C | T | CYB5D2 | p.P173L | 0 | 42 | 0.181818 | 66 | WCM131 | Plasma |
| 17 | 48606459 | G | C | MYCBPAP | p.L921F | 0 | 52 | 0.067568 | 74 | WCM131 | Plasma |
| 17 | 55184494 | G | T | AKAP1 | p.A557S | 0 | 31 | 0.121951 | 41 | WCM131 | Plasma |
| 17 | 6023854 | G | A | WSCD1 | p.R534H | 0 | 75 | 0.223684 | 76 | WCM131 | Plasma |
| 17 | 66039102 | T | C | KPNA2 | p.M218T | 0 | 68 | 0.162162 | 74 | WCM131 | Plasma |
| 17 | 74470523 | G | A | RHBDF2 | p.R495W | 0 | 35 | 0.227273 | 66 | WCM131 | Plasma |
| 17 | 76454668 | C | G | DNAH17 | p.R3314T | 0 | 36 | 0.130435 | 46 | WCM131 | Plasma |
| 17 | 80401897 | G | A | C17orf62 | p.P183S | 0 | 43 | 0.115385 | 78 | WCM131 | Plasma |
| 18 | 13096186 | A | G | CEP192 | p.D1550G | 0 | 33 | 0.160714 | 56 | WCM131 | Plasma |
| 18 | 44773382 | A | T | SKOR2 | p.C725S | 0 | 32 | 0.09375 | 32 | WCM131 | Plasma |
| 18 | 72021145 | T | A | C18orf63 | p.F548Y | 0 | 44 | 0.169811 | 53 | WCM131 | Plasma |
| 19 | 1535205 | G | C | PLK5 | p.G323R | 0 | 35 | 0.137931 | 58 | WCM131 | Plasma |
| 19 | 1650226 | C | A | TCF3 | p.A8S | 0 | 69 | 0.097561 | 82 | WCM131 | Plasma |
| 19 | 19162980 | A | G | ARMC6 | p.K277E | 0 | 39 | 0.222222 | 63 | WCM131 | Plasma |
| 19 | 19427258 | G | A | SUGP1 | p.A60V | 0 | 44 | 0.071429 | 42 | WCM131 | Plasma |
| 19 | 37482151 | A | G | ZNF568 | p.D96G | 0 | 49 | 0.057143 | 70 | WCM131 | Plasma |
| 19 | 37487632 | G | A | ZNF568 | p.A283T | 0 | 32 | 0.12 | 50 | WCM131 | Plasma |
| 19 | 37488299 | A | G | ZNF568 | p.E505G | 0 | 32 | 0.183333 | 60 | WCM131 | Plasma |
| 19 | 37488472 | C | G | ZNF568 | p.P563A | 0 | 47 | 0.115942 | 69 | WCM131 | Plasma |
| 19 | 37488481 | G | C | ZNF568 | p.G566R | 0 | 48 | 0.121212 | 66 | WCM131 | Plasma |
| 19 | 40978640 | G | A | SPTBN4 | p.A38T | 0 | 37 | 0.139535 | 43 | WCM131 | Plasma |
| 19 | 44153202 | A | G | PLAUR | p.I283T | 0 | 33 | 0.210526 | 57 | WCM131 | Plasma |
| 19 | 52097261 | C | G | AC018755.1 | p.R105P | 0 | 40 | 0.142857 | 35 | WCM131 | Plasma |
| 19 | 53643919 | C | T | ZNF347 | p.R721H | 0 | 61 | 0.179775 | 89 | WCM131 | Plasma |
| 19 | 58352806 | T | C | ZNF587B | p.V255A | 0 | 76 | 0.293478 | 92 | WCM131 | Plasma |
| 19 | 58353003 | G | A | ZNF587B | p.A321T | 0 | 72 | 0.264368 | 87 | WCM131 | Plasma |
| 19 | 6468102 | C | T | DENND1C | p.D607N | 0 | 42 | 0.070175 | 57 | WCM131 | Plasma |
| 19 | 7711198 | C | T | STXBP2 | p.R474C | 0 | 43 | 0.131579 | 76 | WCM131 | Plasma |
| 19 | 9407205 | G | A | ZNF699 | p.S292L | 0 | 32 | 0.098361 | 61 | WCM131 | Plasma |
| 19 | 9801365 | T | G | ZNF812 | p.T272P | 0 | 41 | 0.09434 | 53 | WCM131 | Plasma |
| 20 | 1210647 | T | C | RAD21L1 | p.C90R | 0 | 34 | 0.173077 | 52 | WCM131 | Plasma |
| 20 | 1224774 | A | C | RAD21L1 | p.H423P | 0 | 43 | 0.12963 | 54 | WCM131 | Plasma |
| 20 | 31466534 | C | T | EFCAB8 | p.R118C | 0 | 48 | 0.107692 | 65 | WCM131 | Plasma |
| 20 | 56796445 | C | T | ANKRD60 | p.V226M | 0 | 38 | 0.203704 | 54 | WCM131 | Plasma |
| 20 | 61522352 | C | G | DIDO1 | p.Q1167H | 0 | 54 | 0.149425 | 87 | WCM131 | Plasma |
| 2 | 102642617 | T | C | IL1R2 | p.I311T | 0 | 50 | 0.1 | 70 | WCM131 | Plasma |
| 2 | 10959475 | G | C | PDIA6 | p.P34A | 0 | 43 | 0.206897 | 58 | WCM131 | Plasma |
| 2 | 113480339 | G | A | NT5DC4 | p.R147H | 0 | 48 | 0.12069 | 58 | WCM131 | Plasma |
| 2 | 121223274 | C | T | FLJ14816 | p.C138Y | 0 | 32 | 0.294118 | 68 | WCM131 | Plasma |
| 2 | 1320143 | C | T | SNTG2 | p.T495M | 0 | 45 | 0.208333 | 72 | WCM131 | Plasma |
| 2 | 167108386 | G | A | SCN9A | p.R1121W | 0 | 52 | 0.178947 | 95 | WCM131 | Plasma |
| 2 | 170034424 | T | C | LRP2 | p.R3428G | 0 | 44 | 0.115942 | 69 | WCM131 | Plasma |
| 2 | 170488368 | T | A | PPIG | p.F285Y | 0 | 38 | 0.054054 | 74 | WCM131 | Plasma |
| 2 | 172379099 | C | T | CYBRD1 | p.S15L | 0 | 44 | 0.153846 | 65 | WCM131 | Plasma |
| 2 | 175318530 | C | G | GPR155 | p.C604S | 0 | 58 | 0.1125 | 80 | WCM131 | Plasma |
| 2 | 179665289 | C | T | TTN | p.R139Q | 0 | 39 | 0.085714 | 70 | WCM131 | Plasma |
| 2 | 179751485 | T | G | CCDC141 | p.Q507H | 0 | 38 | 0.121212 | 66 | WCM131 | Plasma |
| 2 | 186668935 | A | G | FSIP2 | p.M4968V | 0 | 48 | 0.130435 | 92 | WCM131 | Plasma |
| 22 | 18209989 | G | A | BCL2L13 | p.E383K | 0 | 35 | 0.071429 | 42 | WCM131 | Plasma |
| 22 | 21330818 | G | A | AIFM3 | p.G341S | 0 | 41 | 0.104478 | 67 | WCM131 | Plasma |
| 22 | 25291215 | G | A | SGSM1 | p.R715H | 0 | 31 | 0.066667 | 60 | WCM131 | Plasma |
| 22 | 29138301 | A | G | HSCB | p.Y73C | 0 | 73 | 0.083333 | 72 | WCM131 | Plasma |
| 2 | 231865090 | T | C | SPATA3 | p.I104T | 0 | 32 | 0.097561 | 41 | WCM131 | Plasma |
| 22 | 32113233 | C | T | PRR14L | p.E198K | 0 | 45 | 0.125 | 56 | WCM131 | Plasma |
| 22 | 37398201 | C | A | TEX33 | p.A56S | 0 | 50 | 0.136364 | 88 | WCM131 | Plasma |
| 2 | 238289980 | G | C | COL6A3 | p.T492S | 0 | 31 | 0.142857 | 56 | WCM131 | Plasma |
| 22 | 39003414 | C | T | FAM227A | p.R369Q | 0 | 36 | 0.203125 | 64 | WCM131 | Plasma |
| 22 | 41513682 | A | G | EP300 | p.I196V | 0 | 43 | 0.117647 | 51 | WCM131 | Plasma |
| 2 | 242606080 | G | A | ATG4B | p.V187I | 0 | 33 | 0.108696 | 46 | WCM131 | Plasma |
| 2 | 25013348 | T | G | PTRHD1 | p.I119L | 0 | 60 | 0.166667 | 84 | WCM131 | Plasma |
| 2 | 32733203 | G | A | BIRC6 | p.R3286Q | 0 | 69 | 0.155556 | 90 | WCM131 | Plasma |
| 2 | 36706700 | A | C | CRIM1 | p.H412P | 0 | 32 | 0.128205 | 39 | WCM131 | Plasma |
| 2 | 44174907 | T | C | LRPPRC | p.H643R | 0 | 46 | 0.174603 | 63 | WCM131 | Plasma |
| 3 | 112732817 | A | C | C3orf17 | p.F108L | 0 | 48 | 0.168831 | 77 | WCM131 | Plasma |
| 3 | 131442345 | T | C | CPNE4 | p.H102R | 0 | 35 | 0.094595 | 74 | WCM131 | Plasma |
| 3 | 132242405 | G | A | DNAJC13 | p.V1970M | 0 | 51 | 0.135135 | 74 | WCM131 | Plasma |
| 3 | 132424634 | C | T | NPHP3 | p.A434T | 0 | 63 | 0.086957 | 69 | WCM131 | Plasma |
| 3 | 149485521 | T | C | ANKUB1 | p.M310V | 0 | 43 | 0.169231 | 65 | WCM131 | Plasma |
| 3 | 20042834 | C | T | PP2D1 | p.A260T | 0 | 34 | 0.138462 | 65 | WCM131 | Plasma |
| 3 | 36899394 | C | T | TRANK1 | p.V563M | 0 | 43 | 0.130435 | 69 | WCM131 | Plasma |
| 3 | 49847804 | C | T | UBA7 | p.V509M | 0 | 32 | 0.121951 | 41 | WCM131 | Plasma |
| 3 | 57414097 | T | A | DNAH12 | p.K1754N | 0 | 71 | 0.209877 | 81 | WCM131 | Plasma |
| 3 | 67411166 | A | G | SUCLG2 | p.Y404H | 0 | 52 | 0.111111 | 54 | WCM131 | Plasma |
| 3 | 98235681 | A | G | CLDND1 | p.I195T | 0 | 38 | 0.086957 | 69 | WCM131 | Plasma |
| 4 | 100575711 | C | T | RP11- | p.R32Q | 0 | 63 | 0.153846 | 78 | WCM131 | Plasma |
| | | | | 766F14.2 | | | | | | | |
| 4 | 104084352 | T | C | CENPE | p.Q595R | 0 | 41 | 0.1 | 80 | WCM131 | Plasma |
| 4 | 114458513 | G | T | CAMK2D | p.D167E | 0 | 39 | 0.1 | 60 | WCM131 | Plasma |
| 4 | 135121601 | C | T | PABPC4L | p.V192I | 0 | 56 | 0.297872 | 47 | WCM131 | Plasma |
| 4 | 141383121 | A | T | RP11- | p.L242M | 0 | 74 | 0.130435 | 92 | WCM131 | Plasma |
| | | | | 542P2.1 | | | | | | | |
| 4 | 152203391 | G | A | PRSS48 | p.V103I | 0 | 48 | 0.164179 | 67 | WCM131 | Plasma |
| 4 | 17690072 | T | C | FAM184B | p.Q524R | 0 | 40 | 0.131868 | 91 | WCM131 | Plasma |
| 4 | 20619178 | C | T | SLIT2 | p.A1418V | 0 | 44 | 0.173913 | 69 | WCM131 | Plasma |
| 4 | 2460546 | G | A | RP11- | p.A165T | 0 | 39 | 0.152174 | 46 | WCM131 | Plasma |
| | | | | 503N18.3 | | | | | | | |
| 4 | 26487482 | T | C | CCKAR | p.I135V | 0 | 46 | 0.153846 | 65 | WCM131 | Plasma |
| 4 | 3189510 | T | C | HTT | p.C1708R | 0 | 42 | 0.151515 | 66 | WCM131 | Plasma |
| 4 | 438189 | G | A | ZNF721 | p.P11S | 0 | 40 | 0.096154 | 52 | WCM131 | Plasma |
| 4 | 48851970 | G | A | OCIAD1 | p.C83Y | 0 | 56 | 0.138462 | 65 | WCM131 | Plasma |
| 4 | 79291115 | A | T | FRAS1 | p.D949V | 0 | 48 | 0.152941 | 85 | WCM131 | Plasma |
| 4 | 83776079 | G | A | SEC31A | p.P662L | 0 | 75 | 0.093023 | 86 | WCM131 | Plasma |
| 5 | 112175240 | G | C | APC | p.E1317Q | 0 | 33 | 0.137931 | 58 | WCM131 | Plasma |
| 5 | 140755929 | C | G | PCDHGA6 | p.T760S | 0 | 45 | 0.111111 | 63 | WCM131 | Plasma |
| 5 | 149006768 | A | G | ARHGEF37 | p.I532V | 0 | 45 | 0.145833 | 48 | WCM131 | Plasma |
| 5 | 157102159 | G | A | C5orf52 | p.R91Q | 0 | 90 | 0.17 | 100 | WCM131 | Plasma |
| 5 | 32088073 | A | G | PDZD2 | p.I1507V | 0 | 50 | 0.060606 | 66 | WCM131 | Plasma |
| 5 | 36052004 | A | T | UGT3A2 | p.F93L | 0 | 65 | 0.130435 | 69 | WCM131 | Plasma |
| 5 | 43245944 | G | A | NIM1 | p.D23N | 0 | 34 | 0.150943 | 53 | WCM131 | Plasma |
| 5 | 55237432 | A | C | IL6ST | p.D745E | 0 | 57 | 0.152941 | 85 | WCM131 | Plasma |
| 5 | 74066543 | A | G | NSA2 | p.K144E | 0 | 37 | 0.111111 | 54 | WCM131 | Plasma |
| 5 | 74066544 | A | C | NSA2 | p.K144T | 0 | 36 | 0.115385 | 52 | WCM131 | Plasma |
| 5 | 74442964 | T | C | ANKRD31 | p.R758G | 0 | 38 | 0.177419 | 62 | WCM131 | Plasma |
| 5 | 86703952 | A | T | CCNH | p.S122R | 0 | 44 | 0.092308 | 65 | WCM131 | Plasma |
| 5 | 93987532 | C | A | ANKRD32 | p.S288R | 0 | 52 | 0.109375 | 64 | WCM131 | Plasma |
| 6 | 131540832 | A | C | AKAP7 | p.E245A | 0 | 49 | 0.133333 | 75 | WCM131 | Plasma |
| 6 | 167585654 | G | A | TCP10L2 | p.A8T | 0 | 40 | 0.213115 | 61 | WCM131 | Plasma |
| 6 | 27277665 | G | A | POM121L2 | p.P762L | 0 | 35 | 0.169492 | 59 | WCM131 | Plasma |
| 6 | 28964347 | C | T | ZNF311 | p.M144I | 0 | 32 | 0.207547 | 53 | WCM131 | Plasma |
| 6 | 38029537 | C | T | ZFAND3 | p.P94L | 0 | 50 | 0.160494 | 81 | WCM131 | Plasma |
| 6 | 49479750 | T | C | GLYATL3 | p.M16T | 0 | 45 | 0.173913 | 46 | WCM131 | Plasma |
| 6 | 49489397 | C | A | GLYATL3 | p.A118D | 0 | 57 | 0.162162 | 74 | WCM131 | Plasma |
| 6 | 53385691 | C | T | GCLC | p.G111R | 0 | 50 | 0.162162 | 74 | WCM131 | Plasma |
| 6 | 64694354 | C | T | EYS | p.R2326Q | 0 | 56 | 0.227273 | 88 | WCM131 | Plasma |
| 6 | 65301217 | G | A | EYS | p.R1515W | 0 | 43 | 0.064516 | 62 | WCM131 | Plasma |
| 6 | 65301679 | T | C | EYS | p.I1361V | 0 | 38 | 0.101695 | 59 | WCM131 | Plasma |
| 6 | 65303100 | T | C | EYS | p.I1263V | 0 | 71 | 0.116279 | 86 | WCM131 | Plasma |
| 6 | 74183172 | C | T | MTO1 | p.T207M | 0 | 58 | 0.098765 | 81 | WCM131 | Plasma |
| 6 | 74528127 | A | G | CD109 | p.R1310G | 0 | 49 | 0.12 | 75 | WCM131 | Plasma |
| 6 | 90660390 | C | T | BACH2 | p.A479T | 0 | 47 | 0.123077 | 65 | WCM131 | Plasma |
| 7 | 121733190 | G | A | AASS | p.P560S | 0 | 38 | 0.151515 | 33 | WCM131 | Plasma |
| 7 | 12406989 | C | G | VWDE | p.K964N | 0 | 66 | 0.151515 | 99 | WCM131 | Plasma |
| 7 | 141838362 | C | G | RP11- | p.F327L | 0 | 50 | 0.264368 | 87 | WCM131 | Plasma |
| | | | | 1220K2.2 | | | | | | | |
| 7 | 144094613 | G | T | NOBOX | p.P599H | 0 | 34 | 0.209302 | 43 | WCM131 | Plasma |
| 7 | 150932381 | T | G | CHPF2 | p.F171V | 0 | 33 | 0.159091 | 44 | WCM131 | Plasma |
| 7 | 150932382 | T | C | CHPF2 | p.F171S | 0 | 33 | 0.162791 | 43 | WCM131 | Plasma |
| 7 | 45124286 | A | G | NACAD | p.V498A | 0 | 45 | 0.115385 | 78 | WCM131 | Plasma |
| 7 | 87053289 | G | A | ABCB4 | p.T715I | 0 | 33 | 0.149254 | 67 | WCM131 | Plasma |
| 7 | 87822541 | G | A | ADAM22 | p.R896Q | 0 | 36 | 0.089552 | 67 | WCM131 | Plasma |
| 7 | 91779971 | T | G | LRRD1 | p.I719L | 0 | 48 | 0.2 | 60 | WCM131 | Plasma |
| 7 | 99145678 | C | G | FAM200A | p.R118P | 0 | 31 | 0.189189 | 37 | WCM131 | Plasma |
| 8 | 103851047 | T | G | AZIN1 | p.K125T | 0 | 49 | 0.103448 | 58 | WCM131 | Plasma |
| 8 | 120103453 | A | G | COLEC10 | p.K96E | 0 | 33 | 0.111111 | 63 | WCM131 | Plasma |
| 8 | 133092138 | G | A | HHLA1 | p.P251L | 0 | 51 | 0.328125 | 64 | WCM131 | Plasma |
| 8 | 17143907 | A | T | VPS37A | p.M389L | 0 | 34 | 0.262295 | 61 | WCM131 | Plasma |
| 8 | 17170887 | T | A | MTMR7 | p.M297L | 0 | 31 | 0.207547 | 53 | WCM131 | Plasma |
| 8 | 23150878 | T | G | R3HCC1 | p.L403R | 0 | 32 | 0.135135 | 37 | WCM131 | Plasma |
| 8 | 70476314 | A | G | SULF1 | p.Q35R | 0 | 50 | 0.1 | 60 | WCM131 | Plasma |
| 8 | 90936893 | T | A | OSGIN2 | p.D217E | 0 | 32 | 0.12963 | 54 | WCM131 | Plasma |
| 9 | 100405634 | G | A | NCBP1 | p.V125M | 0 | 48 | 0.088235 | 68 | WCM131 | Plasma |
| 9 | 116346651 | G | A | RGS3 | p.G987S | 0 | 44 | 0.207547 | 53 | WCM131 | Plasma |
| 9 | 134314416 | A | G | PRRC2B | p.Q146R | 0 | 45 | 0.171053 | 76 | WCM131 | Plasma |
| 9 | 34726671 | G | A | FAM205A | p.T189M | 0 | 39 | 0.193548 | 62 | WCM131 | Plasma |
| 9 | 37888096 | T | C | SLC25A51 | p.H151R | 0 | 45 | 0.115942 | 69 | WCM131 | Plasma |
| 9 | 38615698 | C | T | ANKRD18A | p.E130K | 0 | 74 | 0.125 | 96 | WCM131 | Plasma |
| 9 | 5358304 | T | C | PLGRKT | p.M127V | 0 | 42 | 0.081633 | 49 | WCM131 | Plasma |
| 9 | 96312954 | A | G | FAM120A | p.D752G | 0 | 41 | 0.130435 | 69 | WCM131 | Plasma |
| 10 | 124380665 | G | A | DMBT1 | p.V1664M | 0 | 141 | 0.408163 | 98 | WCM131_Z1_1 | Tissue |
| 10 | 12940543 | C | A | CCDC3 | p.R229L | 0 | 106 | 0.120301 | 133 | WCM131_Z1_1 | Tissue |
| 10 | 13325796 | T | C | PHYH | p.E241G | 0 | 71 | 0.636364 | 66 | WCM131_Z1_1 | Tissue |
| 10 | 30915154 | T | C | LYZL2 | p.T106A | 0 | 99 | 0.367925 | 106 | WCM131_Z1_1 | Tissue |
| 10 | 47669277 | C | G | ANTXRL | p.P259A | 0 | 82 | 0.383333 | 60 | WCM131_Z1_1 | Tissue |
| 10 | 50013402 | T | C | WDFY4 | p.S1528P | 0 | 82 | 0.392857 | 84 | WCM131_Z1_1 | Tissue |
| 10 | 50025396 | G | A | WDFY4 | p.R1816Q | 0 | 167 | 0.389262 | 149 | WCM131_Z1_1 | Tissue |
| 10 | 50339754 | G | T | FAM170B | p.D252E | 0 | 380 | 0.362319 | 207 | WCM131_Z1_1 | Tissue |
| 10 | 50339962 | A | T | FAM170B | p.L183Q | 0 | 156 | 0.405405 | 111 | WCM131_Z1_1 | Tissue |
| 10 | 5498098 | G | A | NET1 | p.V416I | 0 | 53 | 0.604651 | 43 | WCM131_Z1_1 | Tissue |
| 10 | 6533711 | G | A | PRKCQ | p.P242S | 0.009346 | 107 | 0.213836 | 159 | WCM131_Z1_1 | Tissue |
| 10 | 76994877 | C | T | COMTD1 | p.V130M | 0 | 34 | 0.543478 | 46 | WCM131_Z1_1 | Tissue |
| 10 | 92677567 | A | T | ANKRD1 | p.H158Q | 0 | 102 | 0.352941 | 119 | WCM131_Z1_1 | Tissue |
| 10 | 94659361 | G | A | EXOC6 | p.E140K | 0 | 36 | 0.15 | 40 | WCM131_Z1_1 | Tissue |
| 1 | 103381192 | C | A | COL11A1 | p.V1271L | 0 | 48 | 0.545455 | 33 | WCM131_Z1_1 | Tissue |
| 11 | 113639557 | C | T | ZW10 | p.E80K | 0 | 106 | 0.625 | 88 | WCM131_Z1_1 | Tissue |
| 11 | 116633658 | C | T | BUD13 | p.R216K | 0 | 78 | 0.451613 | 31 | WCM131_Z1_1 | Tissue |
| 11 | 123600380 | C | T | ZNF202 | p.A186T | 0 | 113 | 0.732394 | 71 | WCM131_Z1_1 | Tissue |
| 11 | 123676829 | G | C | OR6M1 | p.P77A | 0 | 144 | 0.118644 | 59 | WCM131_Z1_1 | Tissue |
| 11 | 124056566 | G | A | OR10D3 | p.R197K | 0 | 94 | 0.111111 | 63 | WCM131_Z1_1 | Tissue |
| 11 | 17393644 | A | G | NCR3LG1 | p.H317R | 0 | 272 | 0.403941 | 203 | WCM131_Z1_1 | Tissue |
| 11 | 17629855 | C | T | OTOG | p.S1402L | 0 | 197 | 0.238806 | 67 | WCM131_Z1_1 | Tissue |
| 11 | 49974261 | T | C | OR4C13 | p.M96T | 0 | 73 | 0.5 | 38 | WCM131_Z1_1 | Tissue |
| 1 | 151638469 | G | A | SNX27 | p.V323M | 0 | 57 | 0.178571 | 84 | WCM131_Z1_1 | Tissue |
| 1 | 152059754 | G | A | TCHHL1 | p.P135L | 0 | 109 | 0.608295 | 217 | WCM131_Z1_1 | Tissue |
| 11 | 5322790 | G | T | OR51B4 | p.Y129* | 0 | 52 | 0.512821 | 39 | WCM131_Z1_1 | Tissue |
| 11 | 55872925 | T | G | OR8H2 | p.M136R | 0 | 147 | 0.183673 | 98 | WCM131_Z1_1 | Tissue |
| 11 | 5653623 | T | G | TRIM34 | p.L21R | 0 | 127 | 0.38961 | 77 | WCM131_Z1_1 | Tissue |
| 1 | 156594952 | C | T | HAPLN2 | p.R267* | 0 | 107 | 0.151899 | 79 | WCM131_Z1_1 | Tissue |
| 11 | 57080394 | A | C | TNKS1BP1 | p.Y590D | 0 | 212 | 0.39823 | 113 | WCM131_Z1_1 | Tissue |
| 1 | 15708540 | A | G | FHAD1 | p.I1325V | 0 | 64 | 0.563636 | 55 | WCM131_Z1_1 | Tissue |
| 1 | 158368948 | G | T | OR10T2 | p.F103L | 0 | 203 | 0.285124 | 242 | WCM131_Z1_1 | Tissue |
| 1 | 160718227 | C | G | SLAMF7 | p.S100* | 0 | 154 | 0.11215 | 107 | WCM131_Z1_1 | Tissue |
| 1 | 160917793 | G | A | ITLN2 | p.R251W | 0 | 93 | 0.15 | 100 | WCM131_Z1_1 | Tissue |
| 11 | 62886800 | G | C | SLC22A24 | p.R172G | 0 | 168 | 0.33945 | 109 | WCM131_Z1_1 | Tissue |
| 11 | 65620182 | C | T | SNX32 | p.R332W | 0 | 84 | 0.383562 | 73 | WCM131_Z1_1 | Tissue |
| 11 | 6569896 | G | A | DNHD1 | p.V2374M | 0 | 72 | 0.803571 | 56 | WCM131_Z1_1 | Tissue |
| 11 | 66358007 | C | T | CCDC87 | p.R827H | 0 | 67 | 0.474576 | 59 | WCM131_Z1_1 | Tissue |
| 11 | 66472775 | G | A | SPTBN2 | p.R658W | 0 | 35 | 0.342857 | 35 | WCM131_Z1_1 | Tissue |
| 11 | 66638660 | C | T | PC | p.V166I | 0 | 67 | 0.292308 | 65 | WCM131_Z1_1 | Tissue |
| 11 | 7022448 | T | C | ZNF214 | p.M156V | 0 | 168 | 0.392593 | 135 | WCM131_Z1_1 | Tissue |
| 1 | 171112490 | C | T | FMO6P | p.Q105* | 0 | 90 | 0.52 | 50 | WCM131_Z1_1 | Tissue |
| 11 | 74203308 | T | A | LIPT2 | p.T190S | 0 | 132 | 0.339623 | 106 | WCM131_Z1_1 | Tissue |
| 11 | 7818410 | C | T | OR5P2 | p.R27Q | 0 | 102 | 0.325843 | 89 | WCM131_Z1_1 | Tissue |
| 1 | 179544941 | G | A | NPHS2 | p.P20L | 0 | 131 | 0.4 | 70 | WCM131_Z1_1 | Tissue |
| 1 | 182369105 | C | T | TEDDM1 | p.M172I | 0 | 58 | 0.285714 | 63 | WCM131_Z1_1 | Tissue |
| 1 | 183201936 | C | T | LAMC2 | p.L722F | 0 | 114 | 0.198347 | 121 | WCM131_Z1_1 | Tissue |
| 11 | 93457532 | C | G | KIAA1731 | p.D2103E | 0 | 230 | 0.565657 | 99 | WCM131_Z1_1 | Tissue |
| 11 | 93458848 | C | G | KIAA1731 | p.A2117G | 0 | 64 | 0.162162 | 37 | WCM131_Z1_1 | Tissue |
| 11 | 95561083 | G | A | CEP57 | p.G340D | 0 | 48 | 0.548387 | 31 | WCM131_Z1_1 | Tissue |
| 1 | 197889142 | C | T | LHX9 | p.A72V | 0 | 52 | 0.131579 | 38 | WCM131_Z1_1 | Tissue |
| 1 | 21009189 | T | G | KIF17 | p.E807A | 0 | 86 | 0.09375 | 64 | WCM131_Z1_1 | Tissue |
| 12 | 104461767 | C | T | HCFC2 | p.P119S | 0 | 53 | 0.295455 | 44 | WCM131_Z1_1 | Tissue |
| 12 | 113384741 | A | G | OAS3 | p.D277G | 0 | 114 | 0.342105 | 76 | WCM131_Z1_1 | Tissue |
| 12 | 128900005 | G | A | TMEM132C | p.V272I | 0 | 75 | 0.447368 | 76 | WCM131_Z1_1 | Tissue |
| 12 | 129153986 | G | A | TMEM132C | p.V444I | 0 | 34 | 0.484848 | 33 | WCM131_Z1_1 | Tissue |
| 12 | 12939892 | G | A | APOLD1 | p.R49Q | 0 | 34 | 0.711111 | 45 | WCM131_Z1_1 | Tissue |
| 1 | 212988440 | T | A | TATDN3 | p.V256E | 0 | 128 | 0.064 | 125 | WCM131_Z1_1 | Tissue |
| 12 | 13061554 | G | A | GPRC5A | p.R124Q | 0 | 100 | 0.565217 | 115 | WCM131_Z1_1 | Tissue |
| 12 | 132401575 | C | T | ULK1 | p.T717M | 0 | 183 | 0.458065 | 155 | WCM131_Z1_1 | Tissue |
| 12 | 132589038 | A | G | EP400NL | p.D158G | 0 | 59 | 0.086957 | 46 | WCM131_Z1_1 | Tissue |
| 12 | 133803551 | G | C | ANHX | p.S230C | 0 | 138 | 0.481481 | 108 | WCM131_Z1_1 | Tissue |
| 1 | 237754229 | C | A | RYR2 | p.T1366N | 0 | 138 | 0.390244 | 82 | WCM131_Z1_1 | Tissue |
| 1 | 238053787 | G | A | ZP4 | p.T50M | 0 | 124 | 0.6625 | 80 | WCM131_Z1_1 | Tissue |
| 12 | 40835734 | C | G | MUC19 | p.I694M | 0 | 54 | 0.435484 | 62 | WCM131_Z1_1 | Tissue |
| 12 | 50744119 | G | A | FAM186A | p.H2166Y | 0 | 101 | 0.27907 | 86 | WCM131_Z1_1 | Tissue |
| 12 | 5155046 | G | A | KCNA5 | p.R578K | 0 | 52 | 0.638095 | 105 | WCM131_Z1_1 | Tissue |
| 12 | 53685589 | G | C | ESPL1 | p.R1879P | 0 | 144 | 0.195652 | 92 | WCM131_Z1_1 | Tissue |
| 12 | 6566837 | C | G | TAPBPL | p.D277E | 0 | 123 | 0.15534 | 206 | WCM131_Z1_1 | Tissue |
| 12 | 65857067 | G | A | MSRB3 | p.A182T | 0 | 71 | 0.5 | 58 | WCM131_Z1_1 | Tissue |
| 12 | 70351684 | C | A | MYRFL | p.L845M | 0 | 53 | 0.285714 | 42 | WCM131_Z1_1 | Tissue |
| 12 | 7252566 | C | T | C1RL | p.A183T | 0 | 363 | 0.677054 | 353 | WCM131_Z1_1 | Tissue |
| 12 | 80655852 | T | C | OTOGL | p.C656R | 0 | 171 | 0.615385 | 65 | WCM131_Z1_1 | Tissue |
| 12 | 80899901 | C | A | PTPRQ | p.Q787K | 0 | 52 | 0.35 | 40 | WCM131_Z1_1 | Tissue |
| 12 | 81627216 | C | G | ACSS3 | p.A562G | 0 | 62 | 0.19403 | 67 | WCM131_Z1_1 | Tissue |
| 12 | 96131658 | C | G | NTN4 | p.G284R | 0 | 106 | 0.307692 | 104 | WCM131_Z1_1 | Tissue |
| 12 | 96934699 | A | G | C12orf55 | p.T671A | 0 | 112 | 0.405172 | 116 | WCM131_Z1_1 | Tissue |
| 12 | 97023939 | A | T | C12orf55 | p.N1391I | 0 | 107 | 0.425532 | 94 | WCM131_Z1_1 | Tissue |
| 12 | 97024355 | A | C | C12orf55 | p.R1414S | 0 | 48 | 0.34375 | 32 | WCM131_Z1_1 | Tissue |
| 13 | 103384712 | G | A | CCDC168 | p.T1483M | 0 | 129 | 0.370968 | 62 | WCM131_Z1_1 | Tissue |
| 13 | 103388015 | C | G | CCDC168 | p.G382A | 0 | 257 | 0.408072 | 223 | WCM131_Z1_1 | Tissue |
| 13 | 112278413 | C | T | RP11- | p.P24L | 0 | 74 | 0.347222 | 72 | WCM131_Z1_1 | Tissue |
| | | | | 65D24.2 | | | | | | | |
| 13 | 113731372 | G | A | MCF2L | p.V533M | 0 | 63 | 0.342105 | 76 | WCM131_Z1_1 | Tissue |
| 13 | 27828398 | G | A | RPL21 | p.G37S | 0 | 112 | 0.277778 | 72 | WCM131_Z1_1 | Tissue |
| 13 | 36764114 | G | A | SOHLH2 | p.L204F | 0 | 101 | 0.297619 | 84 | WCM131_Z1_1 | Tissue |
| 13 | 36886481 | C | T | SPG20 | p.M539I | 0 | 171 | 0.456311 | 103 | WCM131_Z1_1 | Tissue |
| 13 | 39261956 | G | A | FREM2 | p.A159T | 0 | 46 | 0.219512 | 41 | WCM131_Z1_1 | Tissue |
| 13 | 60485919 | A | G | DIAPH3 | p.F773L | 0 | 49 | 0.394737 | 38 | WCM131_Z1_1 | Tissue |
| 14 | 100795151 | C | T | SLC25A47 | p.S139L | 0 | 93 | 0.159091 | 44 | WCM131_Z1_1 | Tissue |
| 14 | 21498871 | C | G | TPPP2 | p.T44R | 0 | 62 | 0.393443 | 61 | WCM131_Z1_1 | Tissue |
| 14 | 21899631 | C | T | CHD8 | p.V58M | 0.006289 | 159 | 0.637363 | 91 | WCM131_Z1_1 | Tissue |
| 14 | 23102704 | G | T | OR6J1 | p.P338Q | 0 | 134 | 0.252336 | 107 | WCM131_Z1_1 | Tissue |
| 14 | 23102882 | C | A | OR6J1 | p.V279L | 0 | 102 | 0.520833 | 48 | WCM131_Z1_1 | Tissue |
| 14 | 23102896 | A | G | OR6J1 | p.L274S | 0 | 227 | 0.519608 | 102 | WCM131_Z1_1 | Tissue |
| 1 | 43240963 | T | G | C1orf50 | p.I166M | 0 | 124 | 0.538462 | 78 | WCM131_Z1_1 | Tissue |
| 1 | 43903355 | A | T | SZT2 | p.Y2070F | 0 | 139 | 0.564706 | 85 | WCM131_Z1_1 | Tissue |
| 14 | 58060664 | G | C | SLC35F4 | p.N130K | 0 | 104 | 0.285714 | 70 | WCM131_Z1_1 | Tissue |
| 14 | 61180321 | G | A | SIX4 | p.S717L | 0 | 97 | 0.271605 | 81 | WCM131_Z1_1 | Tissue |
| 1 | 49119120 | A | G | AGBL4 | p.I243T | 0 | 96 | 0.636364 | 44 | WCM131_Z1_1 | Tissue |
| 14 | 92040673 | C | T | AL133373.1 | p.G95E | 0 | 81 | 0.368421 | 38 | WCM131_Z1_1 | Tissue |
| 14 | 94155163 | T | A | UNC79 | p.H2393Q | 0 | 71 | 0.25 | 88 | WCM131_Z1_1 | Tissue |
| 15 | 29418520 | C | T | FAM189A1 | p.R393H | 0 | 55 | 0.842105 | 38 | WCM131_Z1_1 | Tissue |
| 15 | 29418574 | T | C | FAM189A1 | p.H375R | 0 | 65 | 0.745455 | 55 | WCM131_Z1_1 | Tissue |
| 1 | 53329820 | T | G | ZYG11A | p.H439Q | 0 | 137 | 0.647887 | 71 | WCM131_Z1_1 | Tissue |
| 15 | 33445701 | A | G | FMN1 | p.L472P | 0 | 48 | 0.38 | 50 | WCM131_Z1_1 | Tissue |
| 15 | 41856406 | C | A | TYRO3 | p.S202Y | 0 | 163 | 0.55 | 100 | WCM131_Z1_1 | Tissue |
| 15 | 42342824 | T | G | PLA2G4E | p.E26D | 0 | 190 | 0.381944 | 144 | WCM131_Z1_1 | Tissue |
| 15 | 42528594 | C | G | TMEM87A | p.E298Q | 0 | 44 | 0.47619 | 63 | WCM131_Z1_1 | Tissue |
| 15 | 43817594 | C | T | MAP1A | p.A1308V | 0 | 67 | 0.492958 | 71 | WCM131_Z1_1 | Tissue |
| 15 | 44967847 | A | G | PATL2 | p.L20P | 0 | 64 | 0.227273 | 44 | WCM131_Z1_1 | Tissue |
| 15 | 81214459 | G | A | KIAA1199 | p.R728Q | 0 | 42 | 0.54902 | 51 | WCM131_Z1_1 | Tissue |
| 15 | 83932951 | C | A | BNC1 | p.G351V | 0 | 70 | 0.327586 | 58 | WCM131_Z1_1 | Tissue |
| 15 | 86123809 | C | T | AKAP13 | p.T837M | 0 | 234 | 0.421941 | 237 | WCM131_Z1_1 | Tissue |
| 16 | 11370220 | C | T | PRM2 | p.R3Q | 0 | 145 | 0.401869 | 107 | WCM131_Z1_1 | Tissue |
| 16 | 11931847 | G | A | RSL1D1 | p.P424S | 0 | 141 | 0.140845 | 71 | WCM131_Z1_1 | Tissue |
| 16 | 14345779 | G | A | MKL2 | p.A811T | 0 | 105 | 0.296875 | 64 | WCM131_Z1_1 | Tissue |
| 16 | 1484528 | G | A | CCDC154 | p.R636C | 0 | 133 | 0.125 | 48 | WCM131_Z1_1 | Tissue |
| 16 | 19297219 | T | G | CLEC19A | p.S16A | 0 | 76 | 0.434783 | 69 | WCM131_Z1_1 | Tissue |
| 16 | 23646627 | G | A | PALB2 | p.R414* | 0 | 141 | 0.585106 | 94 | WCM131_Z1_1 | Tissue |
| 16 | 27473628 | A | T | GTF3C1 | p.L2035Q | 0 | 142 | 0.459459 | 74 | WCM131_Z1_1 | Tissue |
| 16 | 28846422 | C | T | ATXN2L | p.T826M | 0 | 74 | 0.385965 | 57 | WCM131_Z1_1 | Tissue |
| 16 | 30772988 | C | T | C16orf93 | p.A28T | 0 | 39 | 0.40625 | 32 | WCM131_Z1_1 | Tissue |
| 16 | 4793135 | G | C | C16orf71 | p.W292S | 0 | 56 | 0.35 | 60 | WCM131_Z1_1 | Tissue |
| 16 | 67911677 | A | G | EDC4 | p.S275G | 0 | 55 | 0.270833 | 48 | WCM131_Z1_1 | Tissue |
| 16 | 85144059 | G | C | FAM92B | p.Q10E | 0 | 182 | 0.1 | 60 | WCM131_Z1_1 | Tissue |
| 16 | 90106769 | A | G | GAS8 | p.Q358R | 0 | 164 | 0.567164 | 67 | WCM131_Z1_1 | Tissue |
| 17 | 10415757 | C | A | MYH1 | p.R372L | 0 | 45 | 0.2 | 70 | WCM131_Z1_1 | Tissue |
| 17 | 1630208 | C | T | WDR81 | p.P652L | 0 | 65 | 0.6 | 40 | WCM131_Z1_1 | Tissue |
| 17 | 17171273 | T | C | COPS3 | p.I123V | 0 | 128 | 0.264463 | 121 | WCM131_Z1_1 | Tissue |
| 17 | 1841037 | G | A | RTN4RL1 | p.R27W | 0 | 67 | 0.396226 | 53 | WCM131_Z1_1 | Tissue |
| 17 | 18881605 | C | A | FAM83G | p.Q458H | 0 | 32 | 0.416667 | 36 | WCM131_Z1_1 | Tissue |
| 17 | 25967610 | C | A | LGALS9 | p.N48K | 0 | 195 | 0.247863 | 117 | WCM131_Z1_1 | Tissue |
| 17 | 26822793 | G | A | SLC13A2 | p.V477M | 0 | 82 | 0.363636 | 77 | WCM131_Z1_1 | Tissue |
| 17 | 28890265 | A | G | TBC1D29 | p.H92R | 0 | 218 | 0.338583 | 127 | WCM131_Z1_1 | Tissue |
| 17 | 29844756 | A | T | RAB11FIP4 | p.M142L | 0 | 170 | 0.310078 | 129 | WCM131_Z1_1 | Tissue |
| 17 | 33875688 | T | A | SLFN14 | p.Y770F | 0 | 66 | 0.405797 | 69 | WCM131_Z1_1 | Tissue |
| 17 | 34122300 | C | T | MMP28 | p.G28R | 0 | 43 | 0.30303 | 33 | WCM131_Z1_1 | Tissue |
| 17 | 38925270 | C | T | KRT26 | p.G350R | 0 | 77 | 0.315068 | 73 | WCM131_Z1_1 | Tissue |
| 17 | 39538524 | C | T | KRT34 | p.C34Y | 0 | 151 | 0.361345 | 119 | WCM131_Z1_1 | Tissue |
| 17 | 39623282 | G | A | KRT32 | p.T99I | 0 | 85 | 0.35 | 60 | WCM131_Z1_1 | Tissue |
| 17 | 4058094 | C | T | CYB5D2 | p.P173L | 0 | 65 | 0.342857 | 70 | WCM131_Z1_1 | Tissue |
| 17 | 41994794 | C | T | FAM215A | p.T39M | 0 | 148 | 0.298851 | 87 | WCM131_Z1_1 | Tissue |
| 17 | 48606459 | G | C | MYCBPAP | p.L921F | 0 | 172 | 0.32 | 125 | WCM131_Z1_1 | Tissue |
| 17 | 55184494 | G | T | AKAP1 | p.A557S | 0 | 75 | 0.25 | 92 | WCM131_Z1_1 | Tissue |
| 17 | 56350817 | G | A | MPO | p.L527F | 0 | 186 | 0.309735 | 113 | WCM131_Z1_1 | Tissue |
| 17 | 6337395 | C | G | AIPL1 | p.M40I | 0 | 203 | 0.283784 | 148 | WCM131_Z1_1 | Tissue |
| 17 | 72613377 | T | C | CD300E | p.T90A | 0 | 107 | 0.164384 | 73 | WCM131_Z1_1 | Tissue |
| 17 | 73843676 | G | A | WBP2 | p.P183S | 0 | 39 | 0.358025 | 81 | WCM131_Z1_1 | Tissue |
| 17 | 74470523 | G | A | RHBDF2 | p.R495W | 0 | 31 | 0.272727 | 55 | WCM131_Z1_1 | Tissue |
| 17 | 80401897 | G | A | C17orf62 | p.P183S | 0 | 67 | 0.653846 | 52 | WCM131_Z1_1 | Tissue |
| 17 | 9631577 | G | A | USP43 | p.R881Q | 0 | 111 | 0.3125 | 64 | WCM131_Z1_1 | Tissue |
| 17 | 9757888 | T | G | GLP2R | p.F194C | 0 | 193 | 0.507143 | 140 | WCM131_Z1_1 | Tissue |
| 18 | 13096186 | A | G | CEP192 | p.D1550G | 0 | 49 | 0.227273 | 66 | WCM131_Z1_1 | Tissue |
| 18 | 44773382 | A | T | SKOR2 | p.C725S | 0 | 52 | 0.43038 | 79 | WCM131_Z1_1 | Tissue |
| 18 | 46146079 | A | G | CTIF | p.D48G | 0 | 57 | 0.341463 | 41 | WCM131_Z1_1 | Tissue |
| 18 | 72021145 | T | A | C18orf63 | p.F548Y | 0 | 89 | 0.380165 | 121 | WCM131_Z1_1 | Tissue |
| 19 | 11350560 | C | T | C19orf80 | p.R83W | 0 | 65 | 0.38 | 50 | WCM131_Z1_1 | Tissue |
| 19 | 14029592 | C | T | CC2D1A | p.S327L | 0 | 31 | 0.282609 | 46 | WCM131_Z1_1 | Tissue |
| 19 | 14164629 | C | T | PALM3 | p.D604N | 0 | 94 | 0.441667 | 120 | WCM131_Z1_1 | Tissue |
| 19 | 14743808 | A | C | EMR3 | p.F523C | 0 | 91 | 0.336842 | 95 | WCM131_Z1_1 | Tissue |
| 19 | 15121702 | G | T | CCDC105 | p.R22L | 0 | 92 | 0.372881 | 59 | WCM131_Z1_1 | Tissue |
| 19 | 1533972 | C | G | PLK5 | p.L253V | 0 | 98 | 0.767123 | 73 | WCM131_Z1_1 | Tissue |
| 19 | 1650226 | C | A | TCF3 | p.A8S | 0 | 160 | 0.507042 | 71 | WCM131_Z1_1 | Tissue |
| 19 | 1754193 | C | G | ONECUT3 | p.L178V | 0 | 45 | 0.333333 | 33 | WCM131_Z1_1 | Tissue |
| 19 | 19162980 | A | G | ARMC6 | p.K277E | 0 | 257 | 0.286585 | 164 | WCM131_Z1_1 | Tissue |
| 19 | 19746315 | G | A | GMIP | p.A490V | 0 | 39 | 0.358974 | 39 | WCM131_Z1_1 | Tissue |
| 19 | 22497445 | G | A | ZNF729 | p.C409Y | 0 | 107 | 0.339623 | 53 | WCM131_Z1_1 | Tissue |
| 19 | 32844888 | C | G | ZNF507 | p.I384M | 0 | 46 | 0.25 | 44 | WCM131_Z1_1 | Tissue |
| 19 | 35597462 | A | G | AC020907.1 | p.E58G | 0 | 69 | 0.760563 | 71 | WCM131_Z1_1 | Tissue |
| 19 | 37487975 | G | A | ZNF568 | p.R397H | 0 | 174 | 0.443396 | 106 | WCM131_Z1_1 | Tissue |
| 19 | 38861424 | A | T | CATSPERG | p.M1158L | 0 | 127 | 0.34 | 100 | WCM131_Z1_1 | Tissue |
| 19 | 40978640 | G | A | SPTBN4 | p.A38T | 0 | 89 | 0.48855 | 131 | WCM131_Z1_1 | Tissue |
| 19 | 4283173 | C | T | SHD | p.R176W | 0 | 48 | 0.315789 | 38 | WCM131_Z1_1 | Tissue |
| 19 | 42906881 | G | C | LIPE | p.R949G | 0 | 74 | 0.233333 | 60 | WCM131_Z1_1 | Tissue |
| 19 | 52097261 | C | G | AC018755.1 | p.R105P | 0 | 58 | 0.2625 | 80 | WCM131_Z1_1 | Tissue |
| 19 | 52570714 | C | G | ZNF841 | p.V25L | 0 | 56 | 0.532258 | 62 | WCM131_Z1_1 | Tissue |
| 19 | 52888245 | A | G | ZNF880 | p.K471R | 0 | 91 | 0.573529 | 136 | WCM131_Z1_1 | Tissue |
| 19 | 53643919 | C | T | ZNF347 | p.R721H | 0 | 80 | 0.645161 | 124 | WCM131_Z1_1 | Tissue |
| 19 | 56028918 | C | T | SSC5D | p.P1092L | 0 | 33 | 0.22807 | 57 | WCM131_Z1_1 | Tissue |
| 19 | 56544053 | C | T | NLRP5 | p.Q785* | 0 | 31 | 0.741935 | 31 | WCM131_Z1_1 | Tissue |
| 19 | 56717333 | C | A | ZSCAN5C | p.S17R | 0 | 167 | 0.192488 | 213 | WCM131_Z1_1 | Tissue |
| 19 | 58352806 | T | C | ZNF587B | p.V255A | 0 | 54 | 0.884211 | 95 | WCM131_Z1_1 | Tissue |
| 19 | 58353003 | G | A | ZNF587B | p.A321T | 0 | 42 | 0.708333 | 96 | WCM131_Z1_1 | Tissue |
| 19 | 6468102 | C | T | DENND1C | p.D607N | 0 | 83 | 0.226667 | 75 | WCM131_Z1_1 | Tissue |
| 19 | 7711198 | C | T | STXBP2 | p.R474C | 0 | 80 | 0.22807 | 57 | WCM131_Z1_1 | Tissue |
| 19 | 9071592 | G | T | MUC16 | p.P5285H | 0 | 54 | 0.258065 | 31 | WCM131_Z1_1 | Tissue |
| 19 | 9407205 | G | A | ZNF699 | p.S292L | 0.006494 | 154 | 0.24359 | 78 | WCM131_Z1_1 | Tissue |
| 19 | 9801365 | T | G | ZNF812 | p.T272P | 0 | 31 | 0.25 | 32 | WCM131_Z1_1 | Tissue |
| 20 | 1224774 | A | C | RAD21L1 | p.H423P | 0 | 111 | 0.481481 | 54 | WCM131_Z1_1 | Tissue |
| 20 | 52645167 | C | A | BCAS1 | p.V163F | 0 | 52 | 0.441176 | 34 | WCM131_Z1_1 | Tissue |
| 20 | 56796445 | C | T | ANKRD60 | p.V226M | 0 | 106 | 0.489362 | 94 | WCM131_Z1_1 | Tissue |
| 20 | 61522352 | C | G | DIDO1 | p.Q1167H | 0 | 88 | 0.37931 | 87 | WCM131_Z1_1 | Tissue |
| 2 | 102642617 | T | C | IL1R2 | p.I311T | 0 | 173 | 0.466216 | 148 | WCM131_Z1_1 | Tissue |
| 2 | 10959475 | G | C | PDIA6 | p.P34A | 0 | 146 | 0.377778 | 135 | WCM131_Z1_1 | Tissue |
| 2 | 113480339 | G | A | NT5DC4 | p.R147H | 0 | 191 | 0.434783 | 138 | WCM131_Z1_1 | Tissue |
| 2 | 121223274 | C | T | FLJ14816 | p.C138Y | 0 | 55 | 0.802817 | 71 | WCM131_Z1_1 | Tissue |
| 2 | 127656810 | G | A | AC114783.1 | p.E55K | 0 | 150 | 0.336066 | 122 | WCM131_Z1_1 | Tissue |
| 21 | 28296469 | G | A | ADAMTS5 | p.T899M | 0 | 71 | 0.428571 | 77 | WCM131_Z1_1 | Tissue |
| 2 | 128408826 | G | A | GPR17 | p.V201M | 0 | 76 | 0.4 | 50 | WCM131_Z1_1 | Tissue |
| 2 | 1320143 | C | T | SNTG2 | p.T495M | 0 | 80 | 0.77027 | 74 | WCM131_Z1_1 | Tissue |
| 2 | 136566805 | A | T | LCT | p.L1038M | 0 | 92 | 0.283019 | 106 | WCM131_Z1_1 | Tissue |
| 21 | 44836887 | G | A | SIK1 | p.P696L | 0 | 31 | 0.447368 | 38 | WCM131_Z1_1 | Tissue |
| 21 | 44841593 | C | T | SIK1 | p.D142N | 0 | 119 | 0.41844 | 141 | WCM131_Z1_1 | Tissue |
| 21 | 47421284 | G | A | COL6A1 | p.R647Q | 0 | 193 | 0.363057 | 157 | WCM131_Z1_1 | Tissue |
| 2 | 152515620 | G | T | NEB | p.Q2012K | 0 | 107 | 0.252874 | 174 | WCM131_Z1_1 | Tissue |
| 2 | 15534469 | A | G | NBAS | p.S1047P | 0 | 191 | 0.351351 | 111 | WCM131_Z1_1 | Tissue |
| 2 | 167108386 | G | A | SCN9A | p.R1121W | 0 | 176 | 0.295858 | 169 | WCM131_Z1_1 | Tissue |
| 2 | 170034424 | T | C | LRP2 | p.R3428G | 0 | 85 | 0.216981 | 106 | WCM131_Z1_1 | Tissue |
| 2 | 170488368 | T | A | PPIG | p.F285Y | 0 | 46 | 0.606061 | 66 | WCM131_Z1_1 | Tissue |
| 2 | 172379099 | C | T | CYBRD1 | p.S15L | 0 | 36 | 0.456522 | 46 | WCM131_Z1_1 | Tissue |
| 2 | 179665289 | C | T | TTN | p.R139Q | 0 | 180 | 0.564103 | 195 | WCM131_Z1_1 | Tissue |
| 2 | 186659184 | C | A | FSIP2 | p.Q2441K | 0 | 41 | 0.516129 | 31 | WCM131_Z1_1 | Tissue |
| 2 | 19553475 | G | A | OSR1 | p.P31L | 0 | 240 | 0.329268 | 164 | WCM131_Z1_1 | Tissue |
| 2 | 215646030 | C | T | BARD1 | p.D190N | 0 | 103 | 0.445652 | 92 | WCM131_Z1_1 | Tissue |
| 2 | 220479221 | C | T | STK11IP | p.S941F | 0 | 78 | 0.314607 | 89 | WCM131_Z1_1 | Tissue |
| 22 | 21330818 | G | A | AIFM3 | p.G341S | 0 | 123 | 0.379032 | 124 | WCM131_Z1_1 | Tissue |
| 22 | 22892382 | A | T | PRAME | p.V240E | 0 | 176 | 0.302083 | 96 | WCM131_Z1_1 | Tissue |
| 22 | 31008882 | G | A | TCN2 | p.G94S | 0 | 126 | 0.425287 | 87 | WCM131_Z1_1 | Tissue |
| 22 | 32081748 | C | A | PRR14L | p.G2074V | 0 | 164 | 0.2 | 115 | WCM131_Z1_1 | Tissue |
| 22 | 32113233 | C | T | PRR14L | p.E198K | 0 | 111 | 0.463158 | 95 | WCM131_Z1_1 | Tissue |
| 22 | 39003414 | C | T | FAM227A | p.R369Q | 0 | 167 | 0.453704 | 108 | WCM131_Z1_1 | Tissue |
| 2 | 239134138 | A | C | AC016757.3 | p.V46G | 0 | 86 | 0.693878 | 49 | WCM131_Z1_1 | Tissue |
| 2 | 240702099 | G | A | AC093802.1 | p.R50H | 0 | 72 | 0.709677 | 62 | WCM131_Z1_1 | Tissue |
| 22 | 41513682 | A | G | EP300 | p.I196V | 0 | 118 | 0.431579 | 95 | WCM131_Z1_1 | Tissue |
| 2 | 241898824 | A | G | AC104809.3 | p.Q911R | 0 | 40 | 0.181818 | 33 | WCM131_Z1_1 | Tissue |
| 2 | 24222717 | A | T | UBXN2A | p.R254* | 0 | 92 | 0.258065 | 62 | WCM131_Z1_1 | Tissue |
| 22 | 42473730 | C | A | FAM109B | p.Q145K | 0 | 40 | 0.5 | 36 | WCM131_Z1_1 | Tissue |
| 2 | 25013348 | T | G | PTRHD1 | p.I119L | 0 | 212 | 0.337931 | 145 | WCM131_Z1_1 | Tissue |
| 2 | 31609376 | G | A | XDH | p.R233C | 0 | 88 | 0.403226 | 62 | WCM131_Z1_1 | Tissue |
| 2 | 32733203 | G | A | BIRC6 | p.R3286Q | 0 | 124 | 0.34965 | 143 | WCM131_Z1_1 | Tissue |
| 2 | 36706700 | A | C | CRIM1 | p.H412P | 0 | 48 | 0.142857 | 42 | WCM131_Z1_1 | Tissue |
| 2 | 40392088 | T | A | SLC8A1 | p.E692V | 0 | 96 | 0.388889 | 72 | WCM131_Z1_1 | Tissue |
| 2 | 73491590 | C | T | FBXO41 | p.R541H | 0 | 74 | 0.202899 | 69 | WCM131_Z1_1 | Tissue |
| 2 | 79254251 | G | T | REG3G | p.S96I | 0 | 128 | 0.327273 | 55 | WCM131_Z1_1 | Tissue |
| 3 | 112732817 | A | C | C3orf17 | p.F108L | 0 | 38 | 0.5 | 38 | WCM131_Z1_1 | Tissue |
| 3 | 130098272 | A | G | COL6A5 | p.I227V | 0 | 184 | 0.386364 | 132 | WCM131_Z1_1 | Tissue |
| 3 | 131442345 | T | C | CPNE4 | p.H102R | 0 | 98 | 0.211268 | 71 | WCM131_Z1_1 | Tissue |
| 3 | 132242405 | G | A | DNAJC13 | p.V1970M | 0 | 66 | 0.491803 | 61 | WCM131_Z1_1 | Tissue |
| 3 | 13413399 | C | T | NUP210 | p.S574N | 0 | 37 | 0.44186 | 43 | WCM131_Z1_1 | Tissue |
| 3 | 149485521 | T | C | ANKUB1 | p.M310V | 0 | 138 | 0.321739 | 115 | WCM131_Z1_1 | Tissue |
| 3 | 151166177 | T | A | IGSF10 | p.D531V | 0 | 58 | 0.229508 | 61 | WCM131_Z1_1 | Tissue |
| 3 | 158363545 | C | G | GFM1 | p.T70S | 0 | 159 | 0.242991 | 107 | WCM131_Z1_1 | Tissue |
| 3 | 170843784 | A | G | TNIK | p.S644P | 0 | 50 | 0.372093 | 43 | WCM131_Z1_1 | Tissue |
| 3 | 178968678 | C | A | KCNMB3 | p.S38I | 0 | 258 | 0.287179 | 195 | WCM131_Z1_1 | Tissue |
| 3 | 36899394 | C | T | TRANK 1 | p.V563M | 0 | 167 | 0.362069 | 116 | WCM131_Z1_1 | Tissue |
| 4 | 100570681 | T | A | RP11- | p.M1709L | 0 | 157 | 0.300971 | 103 | WCM131_Z1_1 | Tissue |
| | | | | 766F14.2 | | | | | | | |
| 4 | 100571001 | T | C | RP11- | p.Q1602R | 0 | 129 | 0.423529 | 85 | WCM131_Z1_1 | Tissue |
| | | | | 766F14.2 | | | | | | | |
| 4 | 100575711 | C | T | RP11- | p.R32Q | 0 | 159 | 0.440945 | 127 | WCM131_Z1_1 | Tissue |
| | | | | 766F14.2 | | | | | | | |
| 4 | 114458513 | G | T | CAMK2D | p.D167E | 0 | 185 | 0.384615 | 117 | WCM131_Z1_1 | Tissue |
| 4 | 126238008 | A | T | FAT4 | p.S148C | 0 | 54 | 0.204545 | 44 | WCM131_Z1_1 | Tissue |
| 4 | 126371478 | A | G | FAT4 | p.S3103G | 0 | 193 | 0.212766 | 94 | WCM131_Z1_1 | Tissue |
| 4 | 135121066 | T | G | PABPC4L | p.H370P | 0 | 141 | 0.831683 | 101 | WCM131_Z1_1 | Tissue |
| 4 | 135121601 | C | T | PABPC4L | p.V192I | 0 | 58 | 0.875 | 64 | WCM131_Z1_1 | Tissue |
| 4 | 152203391 | G | A | PRSS48 | p.V103I | 0 | 111 | 0.432432 | 74 | WCM131_Z1_1 | Tissue |
| 4 | 154557562 | C | T | KIAA0922 | p.T1555M | 0 | 80 | 0.323529 | 68 | WCM131_Z1_1 | Tissue |
| 4 | 164271746 | G | T | NPY5R | p.W107C | 0 | 82 | 0.195122 | 82 | WCM131_Z1_1 | Tissue |
| 4 | 17690072 | T | C | FAM184B | p.Q524R | 0 | 169 | 0.321739 | 115 | WCM131_Z1_1 | Tissue |
| 4 | 20619178 | C | T | SLIT2 | p.A1418V | 0.005155 | 194 | 0.466667 | 150 | WCM131_Z1_1 | Tissue |
| 4 | 2460546 | G | A | RP11- | p.A165T | 0 | 217 | 0.403846 | 208 | WCM131_Z1_1 | Tissue |
| | | | | 503N18.3 | | | | | | | |
| 4 | 3263138 | G | A | MSANTD1 | p.R282Q | 0 | 111 | 0.418919 | 74 | WCM131_Z1_1 | Tissue |
| 4 | 38023317 | C | G | TBC1D1 | p.H396Q | 0 | 70 | 0.050633 | 79 | WCM131_Z1_1 | Tissue |
| 4 | 438189 | G | A | ZNF721 | p.P11S | 0 | 101 | 0.25 | 64 | WCM131_Z1_1 | Tissue |
| 4 | 527677 | T | C | PIGG | p.I881T | 0 | 237 | 0.41206 | 199 | WCM131_Z1_1 | Tissue |
| 4 | 70391429 | G | A | UGT2B4 | p.H7Y | 0 | 92 | 0.465517 | 58 | WCM131_Z1_1 | Tissue |
| 4 | 77177614 | G | A | FAM47E | p.A120T | 0 | 61 | 0.285714 | 35 | WCM131_Z1_1 | Tissue |
| 4 | 77192868 | G | A | FAM47E | p.E273K | 0 | 211 | 0.84127 | 126 | WCM131_Z1_1 | Tissue |
| 4 | 79291115 | A | T | FRAS1 | p.D949V | 0 | 105 | 0.263736 | 91 | WCM131_Z1_1 | Tissue |
| 5 | 102894627 | A | G | NUDT12 | p.L250P | 0 | 60 | 0.363636 | 33 | WCM131_Z1_1 | Tissue |
| 5 | 112175240 | G | C | APC | p.E1317Q | 0 | 79 | 0.22449 | 49 | WCM131_Z1_1 | Tissue |
| 5 | 127744469 | G | A | FBN2 | p.P326S | 0 | 105 | 0.280488 | 82 | WCM131_Z1_1 | Tissue |
| 5 | 130495256 | C | A | HINT1 | p.G89C | 0 | 124 | 0.082192 | 73 | WCM131_Z1_1 | Tissue |
| 5 | 135258105 | G | A | LECT2 | p.P103L | 0 | 188 | 0.752 | 125 | WCM131_Z1_1 | Tissue |
| 5 | 137894313 | C | G | HSPA9 | p.V482L | 0 | 208 | 0.388571 | 175 | WCM131_Z1_1 | Tissue |
| 5 | 138730407 | C | T | PROB1 | p.G122S | 0 | 72 | 0.352941 | 68 | WCM131_Z1_1 | Tissue |
| 5 | 140047844 | G | A | WDR55 | p.E73K | 0 | 96 | 0.444444 | 99 | WCM131_Z1_1 | Tissue |
| 5 | 140581704 | A | T | PCDHB11 | p.N786I | 0 | 87 | 0.280899 | 89 | WCM131_Z1_1 | Tissue |
| 5 | 141019693 | T | A | RELL2 | p.V237E | 0 | 90 | 0.181818 | 88 | WCM131_Z1_1 | Tissue |
| 5 | 141019695 | C | T | RELL2 | p.Q238* | 0 | 105 | 0.170213 | 94 | WCM131_Z1_1 | Tissue |
| 5 | 167675370 | G | A | TENM2 | p.V2476M | 0 | 53 | 0.066667 | 45 | WCM131_Z1_1 | Tissue |
| 5 | 176011974 | G | C | CDHR2 | p.D898H | 0 | 49 | 0.338983 | 59 | WCM131_Z1_1 | Tissue |
| 5 | 23976115 | G | A | C5orf17 | p.V84I | 0.009852 | 203 | 0.763441 | 93 | WCM131_Z1_1 | Tissue |
| 5 | 32088073 | A | G | PDZD2 | p.I1507V | 0 | 80 | 0.412698 | 63 | WCM131_Z1_1 | Tissue |
| 5 | 43245944 | G | A | NIM1 | p.D23N | 0 | 108 | 0.4 | 85 | WCM131_Z1_1 | Tissue |
| 5 | 55237432 | A | C | IL6ST | p.D745E | 0 | 74 | 0.321429 | 112 | WCM131_Z1_1 | Tissue |
| 5 | 71494208 | G | A | MAP1B | p.V1676M | 0 | 92 | 0.417722 | 79 | WCM131_Z1_1 | Tissue |
| 5 | 74442964 | T | C | ANKRD31 | p.R758G | 0 | 80 | 0.352113 | 71 | WCM131_Z1_1 | Tissue |
| 5 | 86703952 | A | T | CCNH | p.S122R | 0 | 59 | 0.483871 | 31 | WCM131_Z1_1 | Tissue |
| 6 | 100390957 | C | T | MCHR2 | p.R152Q | 0 | 116 | 0.46281 | 121 | WCM131_Z1_1 | Tissue |
| 6 | 119137147 | T | C | MCM9 | p.T758A | 0 | 93 | 0.410256 | 78 | WCM131_Z1_1 | Tissue |
| 6 | 13711281 | G | C | RANBP9 | p.R153G | 0 | 130 | 0.283582 | 67 | WCM131_Z1_1 | Tissue |
| 6 | 146625752 | G | A | GRM1 | p.G319E | 0 | 211 | 0.282511 | 223 | WCM131_Z1_1 | Tissue |
| 6 | 166308065 | G | C | SDIM1 | p.S30C | 0 | 101 | 0.272059 | 136 | WCM131_Z1_1 | Tissue |
| 6 | 27278020 | C | A | POM121L2 | p.G644C | 0 | 153 | 0.344262 | 122 | WCM131_Z1_1 | Tissue |
| 6 | 27279632 | C | A | POM121L2 | p.W106C | 0 | 192 | 0.347222 | 144 | WCM131_Z1_1 | Tissue |
| 6 | 28964347 | C | T | ZNF311 | p.M144I | 0 | 41 | 0.307692 | 52 | WCM131_Z1_1 | Tissue |
| 6 | 30139794 | C | T | TRIM15 | p.Q356* | 0 | 67 | 0.547619 | 42 | WCM131_Z1_1 | Tissue |
| 6 | 31625493 | G | C | APOM | p.R178S | 0 | 147 | 0.369863 | 146 | WCM131_Z1_1 | Tissue |
| 6 | 38029537 | C | T | ZFAND3 | p.P94L | 0 | 112 | 0.296875 | 64 | WCM131_Z1_1 | Tissue |
| 6 | 46977400 | C | T | GPR110 | p.V591M | 0 | 82 | 0.416667 | 60 | WCM131_Z1_1 | Tissue |
| 6 | 49479750 | T | C | GLYATL3 | p.M16T | 0 | 109 | 0.349398 | 83 | WCM131_Z1_1 | Tissue |
| 6 | 49489397 | C | A | GLYATL3 | p.A118D | 0 | 93 | 0.338462 | 65 | WCM131_Z1_1 | Tissue |
| 6 | 52141987 | G | A | MCM3 | p.S348F | 0 | 174 | 0.387597 | 129 | WCM131_Z1_1 | Tissue |
| 6 | 5999364 | C | T | NRN1 | p.D92N | 0 | 112 | 0.350515 | 97 | WCM131_Z1_1 | Tissue |
| 6 | 65300143 | G | C | EYS | p.L1873V | 0 | 74 | 0.341463 | 41 | WCM131_Z1_1 | Tissue |
| 6 | 65301408 | A | G | EYS | p.I1451T | 0 | 98 | 0.445545 | 101 | WCM131_Z1_1 | Tissue |
| 6 | 74183172 | C | T | MTO1 | p.T207M | 0 | 110 | 0.346939 | 49 | WCM131_Z1_1 | Tissue |
| 6 | 74528127 | A | G | CD109 | p.R1310G | 0 | 136 | 0.293333 | 75 | WCM131_Z1_1 | Tissue |
| 6 | 97246455 | T | C | GPR63 | p.M385V | 0 | 75 | 0.402985 | 67 | WCM131_Z1_1 | Tissue |
| 7 | 100678784 | G | T | MUC17 | p.D1363Y | 0 | 60 | 0.355556 | 45 | WCM131_Z1_1 | Tissue |
| 7 | 100678785 | A | T | MUC17 | p.D1363V | 0 | 59 | 0.355556 | 45 | WCM131_Z1_1 | Tissue |
| 7 | 104749419 | C | T | KMT2E | p.R1167C | 0 | 76 | 0.597701 | 87 | WCM131_Z1_1 | Tissue |
| 7 | 116340262 | A | G | MET | p.N375S | 0 | 72 | 0.377358 | 53 | WCM131_Z1_1 | Tissue |
| 7 | 141838362 | C | G | RP11- | p.F327L | 0 | 81 | 0.848837 | 86 | WCM131_Z1_1 | Tissue |
| | | | | 1220K2.2 | | | | | | | |
| 7 | 150932381 | T | G | CHPF2 | p.F171V | 0 | 137 | 0.339286 | 112 | WCM131_Z1_1 | Tissue |
| 7 | 150932382 | T | C | CHPF2 | p.F171S | 0 | 137 | 0.339286 | 112 | WCM131_Z1_1 | Tissue |
| 7 | 150934469 | C | T | CHPF2 | p.R341W | 0 | 85 | 0.428571 | 84 | WCM131_Z1_1 | Tissue |
| 7 | 31692357 | C | T | CCDC129 | p.P1017S | 0 | 122 | 0.351064 | 94 | WCM131_Z1_1 | Tissue |
| 7 | 45122324 | C | A | NACAD | p.C1152F | 0 | 37 | 0.369565 | 46 | WCM131_Z1_1 | Tissue |
| 7 | 45122464 | A | C | NACAD | p.D1105E | 0 | 66 | 0.372881 | 59 | WCM131_Z1_1 | Tissue |
| 7 | 45124286 | A | G | NACAD | p.V498A | 0 | 121 | 0.347222 | 72 | WCM131_Z1_1 | Tissue |
| 7 | 45124465 | A | T | NACAD | p.D438E | 0 | 50 | 0.444444 | 36 | WCM131_Z1_1 | Tissue |
| 7 | 48685105 | C | G | ABCA13 | p.I5058M | 0 | 178 | 0.301587 | 126 | WCM131_Z1_1 | Tissue |
| 7 | 5920569 | C | T | OCM | p.Q17* | 0 | 87 | 0.27451 | 51 | WCM131_Z1_1 | Tissue |
| 7 | 86469006 | G | A | GRM3 | p.V726I | 0 | 62 | 0.179487 | 39 | WCM131_Z1_1 | Tissue |
| 7 | 87053289 | G | A | ABCB4 | p.T715I | 0 | 57 | 0.326087 | 46 | WCM131_Z1_1 | Tissue |
| 7 | 87822541 | G | A | ADAM22 | p.R896Q | 0 | 53 | 0.45 | 40 | WCM131_Z1_1 | Tissue |
| 7 | 91779971 | T | G | LRRD1 | p.I719L | 0 | 49 | 0.365854 | 41 | WCM131_Z1_1 | Tissue |
| 7 | 99145678 | C | G | FAM200A | p.R118P | 0 | 58 | 0.421875 | 64 | WCM131_Z1_1 | Tissue |
| 8 | 120103453 | A | G | COLECI10 | p.K96E | 0 | 96 | 0.319149 | 94 | WCM131_Z1_1 | Tissue |
| 8 | 12285202 | T | G | FAM86B2 | p.N286H | 0 | 61 | 0.090909 | 55 | WCM131_Z1_1 | Tissue |
| 8 | 126194399 | A | G | NSMCE2 | p.K107E | 0 | 40 | 0.47541 | 61 | WCM131_Z1_1 | Tissue |
| 8 | 142367903 | G | C | GPR20 | p.L41V | 0 | 97 | 0.285714 | 112 | WCM131_Z1_1 | Tissue |
| 8 | 145141025 | G | T | GPAA1 | p.K621N | 0 | 87 | 0.651685 | 89 | WCM131_Z1_1 | Tissue |
| 8 | 145677818 | G | T | CYHR1 | p.D207E | 0 | 131 | 0.294118 | 119 | WCM131_Z1_1 | Tissue |
| 8 | 17170887 | T | A | MTMR7 | p.M297L | 0 | 107 | 0.324675 | 77 | WCM131_Z1_1 | Tissue |
| 8 | 20074745 | C | G | ATP6V1B2 | p.I392M | 0 | 79 | 0.265625 | 64 | WCM131_Z1_1 | Tissue |
| 8 | 23147564 | G | A | R3HCC1 | p.R185K | 0 | 143 | 0.423423 | 111 | WCM131_Z1_1 | Tissue |
| 8 | 23150878 | T | G | R3HCC1 | p.L403R | 0 | 142 | 0.301887 | 106 | WCM131_Z1_1 | Tissue |
| 8 | 70476314 | A | G | SULF1 | p.Q35R | 0 | 54 | 0.351852 | 54 | WCM131_Z1_1 | Tissue |
| 8 | 90936900 | A | G | OSGIN2 | p.T220A | 0 | 122 | 0.282609 | 92 | WCM131_Z1_1 | Tissue |
| 9 | 123287277 | G | A | CDK5RAP2 | p.T360I | 0 | 249 | 0.386831 | 243 | WCM131_Z1_1 | Tissue |
| 9 | 125377540 | C | T | OR1Q1 | p.P175L | 0 | 167 | 0.575758 | 165 | WCM131_Z1_1 | Tissue |
| 9 | 133305911 | G | A | HMCN2 | p.R566H | 0 | 301 | 0.420765 | 183 | WCM131_Z1_1 | Tissue |
| 9 | 134314416 | A | G | PRRC2B | p.Q146R | 0 | 78 | 0.397727 | 88 | WCM131_Z1_1 | Tissue |
| 9 | 137801702 | G | A | FCN1 | p.A308V | 0 | 110 | 0.395833 | 96 | WCM131_Z1_1 | Tissue |
| 9 | 18706950 | G | A | ADAMTSL1 | p.E594K | 0 | 167 | 0.393162 | 117 | WCM131_Z1_1 | Tissue |
| 9 | 34978079 | G | A | KIAA1045 | p.A392T | 0 | 52 | 0.547619 | 42 | WCM131_Z1_1 | Tissue |
| 9 | 5358304 | T | C | PLGRKT | p.M127V | 0 | 61 | 0.368421 | 38 | WCM131_Z1_1 | Tissue |
| 9 | 96312954 | A | G | FAM120A | p.D752G | 0 | 118 | 0.294118 | 68 | WCM131_Z1_1 | Tissue |
| X | 128924982 | C | A | SASH3 | p.P123T | 0 | 39 | 0.66 | 50 | WCM131_Z1_1 | Tissue |
| X | 152772527 | A | G | BGN | p.I265V | 0 | 42 | 0.857143 | 35 | WCM131_Z1_1 | Tissue |
| X | 19398315 | C | T | MAP3K15 | p.G838S | 0 | 118 | 0.850746 | 67 | WCM131_Z1_1 | Tissue |
| X | 27766045 | A | G | DCAF8L2 | p.T345A | 0 | 74 | 0.791667 | 48 | WCM131_Z1_1 | Tissue |
| X | 65253352 | C | T | VSIG4 | p.V126M | 0 | 104 | 0.802817 | 71 | WCM131_Z1_1 | Tissue |
| 10 | 124380665 | G | A | DMBT1 | p.V1664M | 0 | 141 | 0.141304 | 92 | WCM131_Z2_1 | Tissue |
| 10 | 1279750 | G | C | ADARB2 | p.R467G | 0 | 89 | 0.08547 | 117 | WCM131_Z2_1 | Tissue |
| 10 | 13325796 | T | C | PHYH | p.E241G | 0 | 71 | 0.225806 | 62 | WCM131_Z2_1 | Tissue |
| 10 | 30915154 | T | C | LYZL2 | p.T106A | 0 | 99 | 0.191304 | 115 | WCM131_Z2_1 | Tissue |
| 10 | 47669277 | C | G | ANTXRL | p.P259A | 0 | 82 | 0.194805 | 77 | WCM131_Z2_1 | Tissue |
| 10 | 50013402 | T | C | WDFY4 | p.S1528P | 0 | 82 | 0.211111 | 90 | WCM131_Z2_1 | Tissue |
| 10 | 50025396 | G | A | WDFY4 | p.R1816Q | 0 | 167 | 0.205479 | 146 | WCM131_Z2_1 | Tissue |
| 10 | 50339754 | G | T | FAM170B | p.D252E | 0 | 380 | 0.094637 | 317 | WCM131_Z2_1 | Tissue |
| 10 | 50339962 | A | T | FAM170B | p.L183Q | 0 | 156 | 0.180328 | 122 | WCM131_Z2_1 | Tissue |
| 10 | 5139704 | G | C | AKR1C3 | p.V111L | 0 | 213 | 0.125 | 200 | WCM131_Z2_1 | Tissue |
| 10 | 5416113 | C | T | UCN3 | p.R144C | 0 | 63 | 0.094118 | 85 | WCM131_Z2_1 | Tissue |
| 10 | 5498098 | G | A | NET1 | p.V416I | 0 | 53 | 0.217391 | 46 | WCM131_Z2_1 | Tissue |
| 10 | 6533711 | G | A | PRKCQ | p.P242S | 0.009346 | 107 | 0.18018 | 111 | WCM131_Z2_1 | Tissue |
| 10 | 76994877 | C | T | COMTD1 | p.V130M | 0 | 34 | 0.222222 | 36 | WCM131_Z2_1 | Tissue |
| 10 | 94659361 | G | A | EXOC6 | p.E140K | 0 | 36 | 0.193548 | 62 | WCM131_Z2_1 | Tissue |
| 1 | 103381192 | C | A | COL11A1 | p.V1271L | 0 | 48 | 0.190476 | 63 | WCM131_Z2_1 | Tissue |
| 11 | 100820663 | G | A | ARHGAP42 | p.A380T | 0 | 49 | 0.128205 | 39 | WCM131_Z2_1 | Tissue |
| 11 | 104912206 | G | A | CARD16 | p.P172L | 0 | 211 | 0.193878 | 98 | WCM131_Z2_1 | Tissue |
| 1 | 110714807 | C | T | SLC6A17 | p.R138C | 0 | 93 | 0.180556 | 72 | WCM131_Z2_1 | Tissue |
| 11 | 113639557 | C | T | ZW10 | p.E80K | 0 | 106 | 0.266667 | 90 | WCM131_Z2_1 | Tissue |
| 11 | 114568942 | T | C | NXPE2 | p.V103A | 0 | 46 | 0.135135 | 37 | WCM131_Z2_1 | Tissue |
| 11 | 116633658 | C | T | BUD13 | p.R216K | 0 | 78 | 0.22 | 50 | WCM131_Z2_1 | Tissue |
| 11 | 123600380 | C | T | ZNF202 | p.A186T | 0 | 113 | 0.179487 | 78 | WCM131_Z2_1 | Tissue |
| 11 | 17393644 | A | G | NCR3LG1 | p.H317R | 0 | 272 | 0.128755 | 233 | WCM131_Z2_1 | Tissue |
| 1 | 1237391 | C | A | ACAP3 | p.Q105H | 0 | 84 | 0.084507 | 71 | WCM131_Z2_1 | Tissue |
| 11 | 49075621 | G | A | TRIM64C | p.A330V | 0 | 35 | 0.114286 | 35 | WCM131_Z2_1 | Tissue |
| 11 | 49080616 | T | C | TRIM64C | p.I17V | 0 | 115 | 0.12 | 100 | WCM131_Z2_1 | Tissue |
| 11 | 49974261 | T | C | OR4C13 | p.M96T | 0 | 73 | 0.175676 | 74 | WCM131_Z2_1 | Tissue |
| 1 | 151638469 | G | A | SNX27 | p.V323M | 0 | 57 | 0.092105 | 76 | WCM131_Z2_1 | Tissue |
| 1 | 152059754 | G | A | TCHHL1 | p.P135L | 0 | 109 | 0.464912 | 114 | WCM131_Z2_1 | Tissue |
| 11 | 5322790 | G | T | OR51B4 | p.Y129* | 0 | 52 | 0.14 | 50 | WCM131_Z2_1 | Tissue |
| 11 | 55872925 | T | G | OR8H2 | p.M136R | 0 | 147 | 0.083333 | 72 | WCM131_Z2_1 | Tissue |
| 11 | 5653623 | T | G | TRIM34 | p.L21R | 0 | 127 | 0.116667 | 120 | WCM131_Z2_1 | Tissue |
| 11 | 57080394 | A | C | TNKS1BP1 | p.Y590D | 0 | 212 | 0.096386 | 166 | WCM131_Z2_1 | Tissue |
| 1 | 15708540 | A | G | FHAD1 | p.I1325V | 0 | 64 | 0.155172 | 58 | WCM131_Z2_1 | Tissue |
| 1 | 160718227 | C | G | SLAMF7 | p.S100* | 0 | 154 | 0.070423 | 142 | WCM131_Z2_1 | Tissue |
| 11 | 62886800 | G | C | SLC22A24 | p.R172G | 0 | 168 | 0.16 | 150 | WCM131_Z2_1 | Tissue |
| 11 | 65620182 | C | T | SNX32 | p.R332W | 0 | 84 | 0.191489 | 94 | WCM131_Z2_1 | Tissue |
| 11 | 6569896 | G | A | DNHD1 | p.V2374M | 0 | 72 | 0.333333 | 45 | WCM131_Z2_1 | Tissue |
| 11 | 66638660 | C | T | PC | p.V166I | 0 | 67 | 0.233333 | 90 | WCM131_Z2_1 | Tissue |
| 11 | 68747902 | A | T | MRGPRD | p.L185H | 0 | 185 | 0.093525 | 139 | WCM131_Z2_1 | Tissue |
| 11 | 7022448 | T | C | ZNF214 | p.M156V | 0 | 168 | 0.141176 | 170 | WCM131_Z2_1 | Tissue |
| 1 | 175372507 | C | A | TNR | p.E249* | 0 | 124 | 0.104839 | 124 | WCM131_Z2_1 | Tissue |
| 11 | 7818410 | C | T | OR5P2 | p.R27Q | 0 | 102 | 0.303922 | 102 | WCM131_Z2_1 | Tissue |
| 1 | 179544941 | G | A | NPHS2 | p.P20L | 0 | 131 | 0.145455 | 110 | WCM131_Z2_1 | Tissue |
| 1 | 182369105 | C | T | TEDDM1 | p.M172I | 0 | 58 | 0.163636 | 55 | WCM131_Z2_1 | Tissue |
| 11 | 93457532 | C | G | KIAA1731 | p.D2103E | 0 | 230 | 0.182741 | 197 | WCM131_Z2_1 | Tissue |
| 1 | 197021933 | G | C | F13B | p.N462K | 0 | 94 | 0.125 | 96 | WCM131_Z2_1 | Tissue |
| 1 | 201191923 | G | A | IGFN1 | p.G3420R | 0 | 81 | 0.098592 | 71 | WCM131_Z2_1 | Tissue |
| 1 | 205312591 | A | G | KLHDC8A | p.C48R | 0 | 71 | 0.163934 | 61 | WCM131_Z2_1 | Tissue |
| 12 | 104461767 | C | T | HCFC2 | p.P119S | 0 | 53 | 0.173077 | 52 | WCM131_Z2_1 | Tissue |
| 12 | 113384741 | A | G | OAS3 | p.D277G | 0 | 114 | 0.145161 | 124 | WCM131_Z2_1 | Tissue |
| 12 | 113515832 | C | A | DTX1 | p.T288N | 0 | 100 | 0.28125 | 64 | WCM131_Z2_1 | Tissue |
| 12 | 117658053 | G | T | NOS1 | p.Q1367K | 0 | 79 | 0.193548 | 93 | WCM131_Z2_1 | Tissue |
| 12 | 121437407 | A | G | HNF1A | p.H582R | 0 | 62 | 0.246154 | 65 | WCM131_Z2_1 | Tissue |
| 12 | 128900005 | G | A | TMEM132C | p.V272I | 0 | 75 | 0.212766 | 47 | WCM131_Z2_1 | Tissue |
| 12 | 129153986 | G | A | TMEM132C | p.V444I | 0 | 34 | 0.1 | 40 | WCM131_Z2_1 | Tissue |
| 12 | 12939892 | G | A | APOLD1 | p.R49Q | 0 | 34 | 0.340909 | 44 | WCM131_Z2_1 | Tissue |
| 12 | 13061554 | G | A | GPRC5A | p.R124Q | 0 | 100 | 0.333333 | 90 | WCM131_Z2_1 | Tissue |
| 12 | 132401575 | C | T | ULK1 | p.T717M | 0 | 183 | 0.155914 | 186 | WCM131_Z2_1 | Tissue |
| 12 | 27915718 | A | T | MANSC4 | p.S326T | 0 | 88 | 0.202899 | 69 | WCM131_Z2_1 | Tissue |
| 1 | 237754229 | C | A | RYR2 | p.T1366N | 0 | 138 | 0.066667 | 90 | WCM131_Z2_1 | Tissue |
| 1 | 238053787 | G | A | ZP4 | p.T50M | 0 | 124 | 0.394231 | 104 | WCM131_Z2_1 | Tissue |
| 12 | 40835734 | C | G | MUC19 | p.I694M | 0 | 54 | 0.206349 | 63 | WCM131_Z2_1 | Tissue |
| 12 | 50744119 | G | A | FAM186A | p.H2166Y | 0 | 101 | 0.2 | 130 | WCM131_Z2_1 | Tissue |
| 12 | 5155046 | G | A | KCNA5 | p.R578K | 0 | 52 | 0.266667 | 60 | WCM131_Z2_1 | Tissue |
| 1 | 26189841 | G | T | PAQR7 | p.P164T | 0 | 44 | 0.121951 | 41 | WCM131_Z2_1 | Tissue |
| 12 | 65857067 | G | A | MSRB3 | p.A182T | 0 | 71 | 0.123077 | 65 | WCM131_Z2_1 | Tissue |
| 12 | 70351684 | C | A | MYRFL | p.L845M | 0 | 53 | 0.145455 | 55 | WCM131_Z2_1 | Tissue |
| 12 | 80655852 | T | C | OTOGL | p.C656R | 0 | 171 | 0.196262 | 107 | WCM131_Z2_1 | Tissue |
| 12 | 96934699 | A | G | C12orf55 | p.T671A | 0 | 112 | 0.109091 | 110 | WCM131_Z2_1 | Tissue |
| 12 | 97023939 | A | T | C12orf55 | p.N1391I | 0 | 107 | 0.184 | 125 | WCM131_Z2_1 | Tissue |
| 13 | 103384712 | G | A | CCDC168 | p.T1483M | 0 | 129 | 0.088 | 125 | WCM131_Z2_1 | Tissue |
| 13 | 103388015 | C | G | CCDC168 | p.G382A | 0 | 257 | 0.071111 | 225 | WCM131_Z2_1 | Tissue |
| 13 | 112278413 | C | T | RP11- | p.P24L | 0 | 74 | 0.2 | 65 | WCM131_Z2_1 | Tissue |
| | | | | 65D24.2 | | | | | | | |
| 13 | 113731372 | G | A | MCF2L | p.V533M | 0 | 63 | 0.2 | 70 | WCM131_Z2_1 | Tissue |
| 13 | 25367445 | G | A | RNF17 | p.D401N | 0 | 113 | 0.173913 | 69 | WCM131_Z2_1 | Tissue |
| 13 | 29246531 | G | C | POMP | p.G107A | 0 | 60 | 0.173913 | 46 | WCM131_Z2_1 | Tissue |
| 13 | 36764114 | G | A | SOHLH2 | p.L204F | 0 | 101 | 0.214286 | 126 | WCM131_Z2_1 | Tissue |
| 13 | 36886481 | C | T | SPG20 | p.M539I | 0 | 171 | 0.193103 | 145 | WCM131_Z2_1 | Tissue |
| 13 | 60485919 | A | G | DIAPH3 | p.F773L | 0 | 49 | 0.232558 | 43 | WCM131_Z2_1 | Tissue |
| 1 | 36557401 | A | G | ADPRHL2 | p.Y164C | 0 | 81 | 0.2 | 50 | WCM131_Z2_1 | Tissue |
| 1 | 39799329 | G | T | MACF1 | p.E2362* | 0 | 144 | 0.123188 | 138 | WCM131_Z2_1 | Tissue |
| 1 | 39799330 | A | G | MACF1 | p.E2362G | 0 | 144 | 0.123188 | 138 | WCM131_Z2_1 | Tissue |
| 13 | 99738554 | G | A | DOCK9 | p.R18W | 0 | 44 | 0.172414 | 58 | WCM131_Z2_1 | Tissue |
| 14 | 100795151 | C | T | SLC25A47 | p.S139L | 0 | 93 | 0.053571 | 56 | WCM131_Z2_1 | Tissue |
| 14 | 21899631 | C | T | CHD8 | p.V58M | 0.006289 | 159 | 0.282258 | 124 | WCM131_Z2_1 | Tissue |
| 14 | 23102704 | G | T | OR6J1 | p.P338Q | 0 | 134 | 0.159664 | 119 | WCM131_Z2_1 | Tissue |
| 14 | 23102882 | C | A | OR6J1 | p.V279L | 0 | 102 | 0.222222 | 117 | WCM131_Z2_1 | Tissue |
| 14 | 23102896 | A | G | OR6J1 | p.L274S | 0 | 227 | 0.188341 | 223 | WCM131_Z2_1 | Tissue |
| 14 | 23102969 | G | A | OR6J1 | p.P250S | 0 | 129 | 0.188525 | 122 | WCM131_Z2_1 | Tissue |
| 1 | 43240963 | T | G | C1orf50 | p.I166M | 0 | 124 | 0.16 | 100 | WCM131_Z2_1 | Tissue |
| 1 | 43903355 | A | T | SZT2 | p.Y2070F | 0 | 139 | 0.157534 | 146 | WCM131_Z2_1 | Tissue |
| 14 | 61180321 | G | A | SIX4 | p.S717L | 0 | 97 | 0.156627 | 83 | WCM131_Z2_1 | Tissue |
| 14 | 77948908 | C | G | ISM2 | p.A244P | 0 | 165 | 0.198864 | 176 | WCM131_Z2_1 | Tissue |
| 1 | 49119120 | A | G | AGBL4 | p.I243T | 0 | 96 | 0.186667 | 75 | WCM131_Z2_1 | Tissue |
| 14 | 92040673 | C | T | AL133373.1 | p.G95E | 0 | 81 | 0.21978 | 91 | WCM131_Z2_1 | Tissue |
| 1 | 52747414 | G | A | ZFYVE9 | p.C984Y | 0 | 63 | 0.090909 | 44 | WCM131_Z2_1 | Tissue |
| 15 | 29418520 | C | T | FAM189A1 | p.R393H | 0 | 55 | 0.181818 | 55 | WCM131_Z2_1 | Tissue |
| 15 | 29418574 | T | C | FAM189A1 | p.H375R | 0 | 65 | 0.333333 | 60 | WCM131_Z2_1 | Tissue |
| 1 | 53329820 | T | G | ZYG11A | p.H439Q | 0 | 137 | 0.197917 | 96 | WCM131_Z2_1 | Tissue |
| 15 | 33445701 | A | G | FMN1 | p.L472P | 0 | 48 | 0.184211 | 76 | WCM131_Z2_1 | Tissue |
| 15 | 41856406 | C | A | TYRO3 | p.S202Y | 0 | 163 | 0.139785 | 186 | WCM131_Z2_1 | Tissue |
| 15 | 42150888 | G | A | SPTBN5 | p.T2713I | 0 | 283 | 0.153226 | 248 | WCM131_Z2_1 | Tissue |
| 15 | 42342824 | T | G | PLA2G4E | p.E26D | 0 | 190 | 0.206897 | 203 | WCM131_Z2_1 | Tissue |
| 15 | 43817594 | C | T | MAP1A | p.A1308V | 0 | 67 | 0.183099 | 71 | WCM131_Z2_1 | Tissue |
| 15 | 44967847 | A | G | PATL2 | p.L20P | 0 | 64 | 0.212121 | 66 | WCM131_Z2_1 | Tissue |
| 15 | 70961722 | G | A | UACA | p.T434M | 0 | 40 | 0.15 | 40 | WCM131_Z2_1 | Tissue |
| 1 | 57349308 | C | T | C8A | p.S270L | 0 | 66 | 0.235294 | 51 | WCM131_Z2_1 | Tissue |
| 15 | 81214459 | G | A | KIAA1199 | p.R728Q | 0 | 42 | 0.163265 | 49 | WCM131_Z2_1 | Tissue |
| 15 | 83932951 | C | A | BNC1 | p.G351V | 0 | 70 | 0.166667 | 60 | WCM131_Z2_1 | Tissue |
| 15 | 86123809 | C | T | AKAP13 | p.T837M | 0 | 234 | 0.147959 | 196 | WCM131_Z2_1 | Tissue |
| 16 | 11370220 | C | T | PRM2 | p.R3Q | 0 | 145 | 0.098485 | 132 | WCM131_Z2_1 | Tissue |
| 16 | 14345779 | G | A | MKL2 | p.A811T | 0 | 105 | 0.134831 | 89 | WCM131_Z2_1 | Tissue |
| 16 | 1484528 | G | A | CCDC154 | p.R636C | 0 | 133 | 0.08 | 125 | WCM131_Z2_1 | Tissue |
| 16 | 19297219 | T | G | CLEC19A | p.S16A | 0 | 76 | 0.189873 | 79 | WCM131_Z2_1 | Tissue |
| 16 | 23646627 | G | A | PALB2 | p.R414* | 0 | 141 | 0.230769 | 156 | WCM131_Z2_1 | Tissue |
| 16 | 27473628 | A | T | GTF3C1 | p.L2035Q | 0 | 142 | 0.162162 | 111 | WCM131_Z2_1 | Tissue |
| 16 | 28846422 | C | T | ATXN2L | p.T826M | 0 | 74 | 0.158537 | 82 | WCM131_Z2_1 | Tissue |
| 16 | 30772988 | C | T | C16orf93 | p.A28T | 0 | 39 | 0.222222 | 45 | WCM131_Z2_1 | Tissue |
| 16 | 4793135 | G | C | C16orf71 | p.W292S | 0 | 56 | 0.22619 | 84 | WCM131_Z2_1 | Tissue |
| 1 | 65147732 | C | G | CACHD1 | p.H1177D | 0 | 88 | 0.082474 | 97 | WCM131_Z2_1 | Tissue |
| 16 | 67911677 | A | G | EDC4 | p.S275G | 0 | 55 | 0.156863 | 51 | WCM131_Z2_1 | Tissue |
| 16 | 75263653 | T | G | BCAR1 | p.H790P | 0 | 103 | 0.229885 | 87 | WCM131_Z2_1 | Tissue |
| 16 | 90106769 | A | G | GAS8 | p.Q358R | 0 | 164 | 0.248276 | 145 | WCM131_Z2_1 | Tissue |
| 17 | 1630208 | C | T | WDR81 | p.P652L | 0 | 65 | 0.223684 | 76 | WCM131_Z2_1 | Tissue |
| 17 | 17171273 | T | C | COPS3 | p.I123V | 0 | 128 | 0.153846 | 130 | WCM131_Z2_1 | Tissue |
| 17 | 26822793 | G | A | SLC13A2 | p.V477M | 0 | 82 | 0.192661 | 109 | WCM131_Z2_1 | Tissue |
| 17 | 28890265 | A | G | TBC1D29 | p.H92R | 0 | 218 | 0.211957 | 184 | WCM131_Z2_1 | Tissue |
| 17 | 29844756 | A | T | RAB11FIP4 | p.M142L | 0 | 170 | 0.16 | 150 | WCM131_Z2_1 | Tissue |
| 17 | 38925270 | C | T | KRT26 | p.G350R | 0 | 77 | 0.150943 | 106 | WCM131_Z2_1 | Tissue |
| 17 | 39623282 | G | A | KRT32 | p.T99I | 0 | 85 | 0.142857 | 63 | WCM131_Z2_1 | Tissue |
| 17 | 4058094 | C | T | CYB5D2 | p.P173L | 0 | 65 | 0.2 | 75 | WCM131_Z2_1 | Tissue |
| 17 | 41994794 | C | T | FAM215A | p.T39M | 0 | 148 | 0.218487 | 119 | WCM131_Z2_1 | Tissue |
| 17 | 48606459 | G | C | MYCBPAP | p.L921F | 0 | 172 | 0.173554 | 121 | WCM131_Z2_1 | Tissue |
| 1 | 74946052 | C | A | LRRC53 | p.R230I | 0 | 170 | 0.178161 | 174 | WCM131_Z2_1 | Tissue |
| 17 | 55184494 | G | T | AKAP1 | p.A557S | 0 | 75 | 0.219178 | 73 | WCM131_Z2_1 | Tissue |
| 17 | 60767597 | G | A | MRC2 | p.E1275K | 0 | 298 | 0.11215 | 214 | WCM131_Z2_1 | Tissue |
| 17 | 63173912 | G | C | RGS9 | p.K215N | 0 | 124 | 0.152542 | 118 | WCM131_Z2_1 | Tissue |
| 17 | 72613377 | T | C | CD300E | p.T90A | 0 | 107 | 0.160494 | 81 | WCM131_Z2_1 | Tissue |
| 17 | 80401897 | G | A | C17orf62 | p.P183S | 0 | 67 | 0.088235 | 68 | WCM131_Z2_1 | Tissue |
| 17 | 9631577 | G | A | USP43 | p.R881Q | 0 | 111 | 0.135135 | 74 | WCM131_Z2_1 | Tissue |
| 17 | 9757888 | T | G | GLP2R | p.F194C | 0 | 193 | 0.142857 | 154 | WCM131_Z2_1 | Tissue |
| 18 | 13096186 | A | G | CEP192 | p.D1550G | 0 | 49 | 0.121951 | 41 | WCM131_Z2_1 | Tissue |
| 18 | 24524310 | C | T | CHST9 | p.E75K | 0 | 49 | 0.3125 | 48 | WCM131_Z2_1 | Tissue |
| 18 | 44773382 | A | T | SKOR2 | p.C725S | 0 | 52 | 0.140845 | 71 | WCM131_Z2_1 | Tissue |
| 18 | 46146079 | A | G | CTIF | p.D48G | 0 | 57 | 0.15 | 40 | WCM131_Z2_1 | Tissue |
| 18 | 47812423 | G | T | CXXC1 | p.S143Y | 0 | 148 | 0.216561 | 157 | WCM131_Z2_1 | Tissue |
| 18 | 5890777 | T | A | TMEM200C | p.E429V | 0 | 42 | 0.319149 | 47 | WCM131_Z2_1 | Tissue |
| 18 | 72021145 | T | A | C18orf63 | p.F548Y | 0 | 89 | 0.245902 | 122 | WCM131_Z2_1 | Tissue |
| 19 | 11350560 | C | T | C19orf80 | p.R83W | 0 | 65 | 0.196078 | 51 | WCM131_Z2_1 | Tissue |
| 19 | 14029592 | C | T | CC2D1A | p.S327L | 0 | 31 | 0.186047 | 43 | WCM131_Z2_1 | Tissue |
| 19 | 14743808 | A | C | EMR3 | p.F523C | 0 | 91 | 0.15625 | 64 | WCM131_Z2_1 | Tissue |
| 19 | 1533972 | C | G | PLK5 | p.L253V | 0 | 98 | 0.306667 | 75 | WCM131_Z2_1 | Tissue |
| 19 | 1535205 | G | C | PLK5 | p.G323R | 0 | 76 | 0.30137 | 73 | WCM131_Z2_1 | Tissue |
| 19 | 1650226 | C | A | TCF3 | p.A8S | 0 | 160 | 0.111111 | 135 | WCM131_Z2_1 | Tissue |
| 19 | 1754193 | C | G | ONECUT3 | p.L178V | 0 | 45 | 0.142857 | 42 | WCM131_Z2_1 | Tissue |
| 19 | 19162980 | A | G | ARMC6 | p.K277E | 0 | 257 | 0.124464 | 233 | WCM131_Z2_1 | Tissue |
| 19 | 19746315 | G | A | GMIP | p.A490V | 0 | 39 | 0.157895 | 38 | WCM131_Z2_1 | Tissue |
| 19 | 22497445 | G | A | ZNF729 | p.C409Y | 0 | 107 | 0.108434 | 83 | WCM131_Z2_1 | Tissue |
| 19 | 32844888 | C | G | ZNF507 | p.I384M | 0 | 46 | 0.128205 | 39 | WCM131_Z2_1 | Tissue |
| 19 | 37487975 | G | A | ZNF568 | p.R397H | 0 | 174 | 0.207692 | 130 | WCM131_Z2_1 | Tissue |
| 19 | 38861424 | A | T | CATSPERG | p.M1158L | 0 | 127 | 0.197368 | 76 | WCM131_Z2_1 | Tissue |
| 19 | 40978640 | G | A | SPTBN4 | p.A38T | 0 | 89 | 0.22449 | 98 | WCM131_Z2_1 | Tissue |
| 19 | 4283173 | C | T | SHD | p.R176W | 0 | 48 | 0.081081 | 37 | WCM131_Z2_1 | Tissue |
| 19 | 52097261 | C | G | AC018755.1 | p.R105P | 0 | 58 | 0.107143 | 56 | WCM131_Z2_1 | Tissue |
| 19 | 52570714 | C | G | ZNF841 | p.V25L | 0 | 56 | 0.381818 | 55 | WCM131_Z2_1 | Tissue |
| 19 | 52888245 | A | G | ZNF880 | p.K471R | 0 | 91 | 0.40625 | 128 | WCM131_Z2_1 | Tissue |
| 19 | 53643919 | C | T | ZNF347 | p.R721H | 0 | 80 | 0.458824 | 85 | WCM131_Z2_1 | Tissue |
| 19 | 56028918 | C | T | SSC5D | p.P1092L | 0 | 33 | 0.146341 | 41 | WCM131_Z2_1 | Tissue |
| 19 | 56544053 | C | T | NLRP5 | p.Q785* | 0 | 31 | 0.26087 | 46 | WCM131_Z2_1 | Tissue |
| 19 | 56717333 | C | A | ZSCAN5C | p.S17R | 0 | 167 | 0.063107 | 206 | WCM131_Z2_1 | Tissue |
| 19 | 57335912 | C | T | PEG3 | p.G38S | 0 | 164 | 0.125 | 184 | WCM131_Z2_1 | Tissue |
| 19 | 58352806 | T | C | ZNF587B | p.V255A | 0 | 54 | 0.512821 | 39 | WCM131_Z2_1 | Tissue |
| 19 | 58353003 | G | A | ZNF587B | p.A321T | 0 | 42 | 0.461538 | 78 | WCM131_Z2_1 | Tissue |
| 19 | 6468102 | C | T | DENND1C | p.D607N | 0 | 83 | 0.090909 | 66 | WCM131_Z2_1 | Tissue |
| 19 | 7711198 | C | T | STXBP2 | p.R474C | 0 | 80 | 0.105263 | 95 | WCM131_Z2_1 | Tissue |
| 19 | 8953449 | G | T | MBD3L1 | p.S32I | 0 | 175 | 0.148387 | 155 | WCM131_Z2_1 | Tissue |
| 19 | 9407205 | G | A | ZNF699 | p.S292L | 0.006494 | 154 | 0.132743 | 113 | WCM131_Z2_1 | Tissue |
| 20 | 1224774 | A | C | RAD21L1 | p.H423P | 0 | 111 | 0.166667 | 54 | WCM131_Z2_1 | Tissue |
| 20 | 52645167 | C | A | BCAS1 | p.V163F | 0 | 52 | 0.222222 | 45 | WCM131_Z2_1 | Tissue |
| 20 | 56796445 | C | T | ANKRD60 | p.V226M | 0 | 106 | 0.17094 | 117 | WCM131_Z2_1 | Tissue |
| 20 | 61522352 | C | G | DIDO1 | p.Q1167H | 0 | 88 | 0.105263 | 95 | WCM131_Z2_1 | Tissue |
| 2 | 102642617 | T | C | IL1R2 | p.I311T | 0 | 173 | 0.25 | 144 | WCM131_Z2_1 | Tissue |
| 2 | 10959475 | G | C | PDIA6 | p.P34A | 0 | 146 | 0.236025 | 161 | WCM131_Z2_1 | Tissue |
| 2 | 113480339 | G | A | NT5DC4 | p.R147H | 0 | 191 | 0.198718 | 156 | WCM131_Z2_1 | Tissue |
| 2 | 121223274 | C | T | FLJ14816 | p.C138Y | 0 | 55 | 0.365385 | 52 | WCM131_Z2_1 | Tissue |
| 21 | 28296469 | G | A | ADAMTS5 | p.T899M | 0 | 71 | 0.083333 | 72 | WCM131_Z2_1 | Tissue |
| 2 | 128408826 | G | A | GPR17 | p.V201M | 0 | 76 | 0.237288 | 59 | WCM131_Z2_1 | Tissue |
| 2 | 1320143 | C | T | SNTG2 | p.T495M | 0 | 80 | 0.598039 | 102 | WCM131_Z2_1 | Tissue |
| 21 | 44841593 | C | T | SIK1 | p.D142N | 0 | 119 | 0.201493 | 134 | WCM131_Z2_1 | Tissue |
| 21 | 47421284 | G | A | COL6A1 | p.R647Q | 0 | 193 | 0.163636 | 165 | WCM131_Z2_1 | Tissue |
| 2 | 152515620 | G | T | NEB | p.Q2012K | 0 | 107 | 0.188976 | 127 | WCM131_Z2_1 | Tissue |
| 2 | 15534469 | A | G | NBAS | p.S1047P | 0 | 191 | 0.127451 | 204 | WCM131_Z2_1 | Tissue |
| 2 | 1652072 | C | A | PXDN | p.Q1160H | 0 | 152 | 0.136126 | 191 | WCM131_Z2_1 | Tissue |
| 2 | 167108386 | G | A | SCN9A | p.R1121W | 0 | 176 | 0.186047 | 129 | WCM131_Z2_1 | Tissue |
| 2 | 170034424 | T | C | LRP2 | p.R3428G | 0 | 85 | 0.181818 | 66 | WCM131_Z2_1 | Tissue |
| 2 | 170488368 | T | A | PPIG | p.F285Y | 0 | 46 | 0.275862 | 58 | WCM131_Z2_1 | Tissue |
| 2 | 173292556 | G | T | ITGA6 | p.A14S | 0 | 33 | 0.081081 | 37 | WCM131_Z2_1 | Tissue |
| 2 | 179665289 | C | T | TTN | p.R139Q | 0 | 180 | 0.190722 | 194 | WCM131_Z2_1 | Tissue |
| 2 | 186659184 | C | A | FSIP2 | p.Q2441K | 0 | 41 | 0.142857 | 56 | WCM131_Z2_1 | Tissue |
| 2 | 19553475 | G | A | OSR1 | p.P31L | 0 | 240 | 0.163743 | 171 | WCM131_Z2_1 | Tissue |
| 2 | 196619105 | T | C | DNAH7 | p.Y3907C | 0 | 49 | 0.266667 | 60 | WCM131_Z2_1 | Tissue |
| 2 | 207310189 | T | G | ADAM23 | p.S125A | 0 | 86 | 0.242105 | 95 | WCM131_Z2_1 | Tissue |
| 2 | 215646030 | C | T | BARD1 | p.D190N | 0 | 103 | 0.153846 | 130 | WCM131_Z2_1 | Tissue |
| 2 | 220479221 | C | T | STK11IP | p.S941F | 0 | 78 | 0.108434 | 83 | WCM131_Z2_1 | Tissue |
| 22 | 21330818 | G | A | AIFM3 | p.G341S | 0 | 123 | 0.165414 | 133 | WCM131_Z2_1 | Tissue |
| 22 | 22892382 | A | T | PRAME | p.V240E | 0 | 176 | 0.202247 | 178 | WCM131_Z2_1 | Tissue |
| 2 | 228131783 | C | G | COL4A3 | p.H495D | 0 | 186 | 0.139073 | 151 | WCM131_Z2_1 | Tissue |
| 22 | 31008882 | G | A | TCN2 | p.G94S | 0 | 126 | 0.275591 | 127 | WCM131_Z2_1 | Tissue |
| 2 | 231865090 | T | C | SPATA3 | p.I104T | 0 | 71 | 0.073529 | 68 | WCM131_Z2_1 | Tissue |
| 22 | 32081748 | C | A | PRR14L | p.G2074V | 0 | 164 | 0.171875 | 192 | WCM131_Z2_1 | Tissue |
| 22 | 32113233 | C | T | PRR14L | p.E198K | 0 | 111 | 0.156863 | 102 | WCM131_Z2_1 | Tissue |
| 2 | 238289984 | C | G | COL6A3 | p.D491H | 0 | 82 | 0.146067 | 89 | WCM131_Z2_1 | Tissue |
| 22 | 39003414 | C | T | FAM227A | p.R369Q | 0 | 167 | 0.20438 | 137 | WCM131_Z2_1 | Tissue |
| 2 | 239133980 | C | T | AC016757.3 | p.G99R | 0 | 69 | 0.162162 | 74 | WCM131_Z2_1 | Tissue |
| 2 | 239134138 | A | C | AC016757.3 | p.V46G | 0 | 86 | 0.161616 | 99 | WCM131_Z2_1 | Tissue |
| 2 | 240036889 | C | A | HDAC4 | p.G546W | 0 | 110 | 0.138298 | 94 | WCM131_Z2_1 | Tissue |
| 2 | 240702099 | G | A | AC093802.1 | p.R50H | 0 | 72 | 0.147059 | 68 | WCM131_Z2_1 | Tissue |
| 2 | 241872597 | A | G | AC104809.3 | p.Q328R | 0 | 48 | 0.145833 | 48 | WCM131_Z2_1 | Tissue |
| 2 | 24222717 | A | T | UBXN2A | p.R254* | 0 | 92 | 0.128571 | 70 | WCM131_Z2_1 | Tissue |
| 2 | 242606080 | G | A | ATG4B | p.V187I | 0 | 49 | 0.128205 | 39 | WCM131_Z2_1 | Tissue |
| 2 | 25013348 | T | G | PTRHD1 | p.I119L | 0 | 212 | 0.192513 | 187 | WCM131_Z2_1 | Tissue |
| 2 | 31609376 | G | A | XDH | p.R233C | 0 | 88 | 0.184466 | 103 | WCM131_Z2_1 | Tissue |
| 2 | 32733203 | G | A | BIRC6 | p.R3286Q | 0 | 124 | 0.230216 | 139 | WCM131_Z2_1 | Tissue |
| 2 | 40392088 | T | A | SLC8A1 | p.E692V | 0 | 96 | 0.1875 | 80 | WCM131_Z2_1 | Tissue |
| 2 | 73491590 | C | T | FBXO41 | p.R541H | 0 | 74 | 0.142857 | 49 | WCM131_Z2_1 | Tissue |
| 2 | 79254251 | G | T | REG3G | p.S96I | 0 | 128 | 0.144068 | 118 | WCM131_Z2_1 | Tissue |
| 3 | 112732817 | A | C | C3orf17 | p.F108L | 0 | 38 | 0.1875 | 48 | WCM131_Z2_1 | Tissue |
| 3 | 130098272 | A | G | COL6A5 | p.I227V | 0 | 184 | 0.152174 | 184 | WCM131_Z2_1 | Tissue |
| 3 | 132242405 | G | A | DNAJC13 | p.V1970M | 0 | 66 | 0.089888 | 89 | WCM131_Z2_1 | Tissue |
| 3 | 13413399 | C | T | NUP210 | p.S574N | 0 | 37 | 0.066667 | 60 | WCM131_Z2_1 | Tissue |
| 3 | 149485521 | T | C | ANKUB1 | p.M310V | 0 | 138 | 0.151079 | 139 | WCM131_Z2_1 | Tissue |
| 3 | 36899394 | C | T | TRANK1 | p.V563M | 0 | 167 | 0.172932 | 133 | WCM131_Z2_1 | Tissue |
| 4 | 100570681 | T | A | RP11- | p.M1709L | 0 | 157 | 0.164179 | 134 | WCM131_Z2_1 | Tissue |
| | | | | 766F14.2 | | | | | | | |
| 4 | 100571001 | T | C | RP11- | p.Q1602R | 0 | 129 | 0.145631 | 103 | WCM131_Z2_1 | Tissue |
| | | | | 766F14.2 | | | | | | | |
| 4 | 100575711 | C | T | RP11- | p.R32Q | 0 | 159 | 0.144509 | 173 | WCM131_Z2_1 | Tissue |
| | | | | 766F14.2 | | | | | | | |
| 4 | 114458513 | G | T | CAMK2D | p.D167E | 0 | 185 | 0.168831 | 154 | WCM131_Z2_1 | Tissue |
| 4 | 126238008 | A | T | FAT4 | p.S148C | 0 | 54 | 0.093023 | 43 | WCM131_Z2_1 | Tissue |
| 4 | 126371478 | A | G | FAT4 | p.S3103G | 0 | 193 | 0.118012 | 161 | WCM131_Z2_1 | Tissue |
| 4 | 135121066 | T | G | PABPC4L | p.H370P | 0 | 141 | 0.4 | 120 | WCM131_Z2_1 | Tissue |
| 4 | 135121601 | C | T | PABPC4L | p.V192I | 0 | 58 | 0.301205 | 83 | WCM131_Z2_1 | Tissue |
| 4 | 152203391 | G | A | PRSS48 | p.V103I | 0 | 111 | 0.346154 | 104 | WCM131_Z2_1 | Tissue |
| 4 | 154557562 | C | T | KIAA0922 | p.T1555M | 0 | 80 | 0.168317 | 101 | WCM131_Z2_1 | Tissue |
| 4 | 15516409 | C | A | CC2D2A | p.A266E | 0 | 79 | 0.236364 | 55 | WCM131_Z2_1 | Tissue |
| 4 | 17690072 | T | C | FAM184B | p.Q524R | 0 | 169 | 0.119403 | 134 | WCM131_Z2_1 | Tissue |
| 4 | 20619178 | C | T | SLIT2 | p.A1418V | 0.005155 | 194 | 0.193182 | 176 | WCM131_Z2_1 | Tissue |
| 4 | 3263138 | G | A | MSANTD1 | p.R282Q | 0 | 111 | 0.116667 | 60 | WCM131_Z2_1 | Tissue |
| 4 | 527677 | T | C | PIGG | p.I881T | 0 | 237 | 0.189076 | 238 | WCM131_Z2_1 | Tissue |
| 4 | 54967809 | C | A | GSX2 | p.T212K | 0 | 93 | 0.19403 | 67 | WCM131_Z2_1 | Tissue |
| 4 | 70391429 | G | A | UGT2B4 | p.H7Y | 0 | 92 | 0.236559 | 93 | WCM131_Z2_1 | Tissue |
| 4 | 77192868 | G | A | FAM47E | p.E273K | 0 | 211 | 0.388889 | 180 | WCM131_Z2_1 | Tissue |
| 4 | 79291115 | A | T | FRAS1 | p.D949V | 0 | 105 | 0.190083 | 121 | WCM131_Z2_1 | Tissue |
| 5 | 102894627 | A | G | NUDT12 | p.L250P | 0 | 60 | 0.061224 | 49 | WCM131_Z2_1 | Tissue |
| 5 | 112175240 | G | C | APC | p.E1317Q | 0 | 79 | 0.192308 | 52 | WCM131_Z2_1 | Tissue |
| 5 | 127744469 | G | A | FBN2 | p.P326S | 0 | 105 | 0.194175 | 103 | WCM131_Z2_1 | Tissue |
| 5 | 135258105 | G | A | LECT2 | p.P103L | 0 | 188 | 0.413333 | 150 | WCM131_Z2_1 | Tissue |
| 5 | 138730407 | C | T | PROB1 | p.G122S | 0 | 72 | 0.15942 | 69 | WCM131_Z2_1 | Tissue |
| 5 | 140047844 | G | A | WDR55 | p.E73K | 0 | 96 | 0.106667 | 75 | WCM131_Z2_1 | Tissue |
| 5 | 140581704 | A | T | PCDHB11 | p.N786I | 0 | 87 | 0.15 | 100 | WCM131_Z2_1 | Tissue |
| 5 | 141019693 | T | A | RELL2 | p.V237E | 0 | 90 | 0.078947 | 76 | WCM131_Z2_1 | Tissue |
| 5 | 141019695 | C | T | RELL2 | p.Q238* | 0 | 105 | 0.1 | 80 | WCM131_Z2_1 | Tissue |
| 5 | 171201499 | G | C | CTB-78H18.1 | p.G97A | 0 | 93 | 0.072289 | 83 | WCM131_Z2_1 | Tissue |
| 5 | 23976115 | G | A | C5orf17 | p.V84I | 0.009852 | 203 | 0.365854 | 123 | WCM131_Z2_1 | Tissue |
| 5 | 32088073 | A | G | PDZD2 | p.I1507V | 0 | 80 | 0.081395 | 86 | WCM131_Z2_1 | Tissue |
| 5 | 36052004 | A | T | UGT3A2 | p.F93L | 0 | 114 | 0.211538 | 104 | WCM131_Z2_1 | Tissue |
| 5 | 43245944 | G | A | NIM1 | p.D23N | 0 | 108 | 0.165138 | 109 | WCM131_Z2_1 | Tissue |
| 5 | 55237432 | A | C | IL6ST | p.D745E | 0 | 74 | 0.234043 | 94 | WCM131_Z2_1 | Tissue |
| 5 | 60840045 | T | A | ZSWIM6 | p.H1183Q | 0 | 168 | 0.152047 | 171 | WCM131_Z2_1 | Tissue |
| 5 | 71494208 | G | A | MAP1B | p.V1676M | 0 | 92 | 0.13 | 100 | WCM131_Z2_1 | Tissue |
| 5 | 86703952 | A | T | CCNH | p.S122R | 0 | 59 | 0.181818 | 44 | WCM131_Z2_1 | Tissue |
| 5 | 93753017 | C | T | KIAA0825 | p.A851T | 0 | 149 | 0.203593 | 167 | WCM131_Z2_1 | Tissue |
| 5 | 93987532 | C | A | ANKRD32 | p.S288R | 0 | 145 | 0.137097 | 124 | WCM131_Z2_1 | Tissue |
| 6 | 100390957 | C | T | MCHR2 | p.R152Q | 0 | 116 | 0.197368 | 76 | WCM131_Z2_1 | Tissue |
| 6 | 111912730 | C | A | TRAF3IP2 | p.R196L | 0 | 37 | 0.075 | 40 | WCM131_Z2_1 | Tissue |
| 6 | 119137147 | T | C | MCM9 | p.T758A | 0 | 93 | 0.261364 | 88 | WCM131_Z2_1 | Tissue |
| 6 | 135323972 | C | T | HBS1L | p.V147I | 0 | 94 | 0.072289 | 83 | WCM131_Z2_1 | Tissue |
| 6 | 13711281 | G | C | RANBP9 | p.R153G | 0 | 130 | 0.12 | 125 | WCM131_Z2_1 | Tissue |
| 6 | 159420722 | G | T | RSPH3 | p.P96H | 0 | 38 | 0.138889 | 36 | WCM131_Z2_1 | Tissue |
| 6 | 166308065 | G | C | SDIM1 | p.S30C | 0 | 101 | 0.148387 | 155 | WCM131_Z2_1 | Tissue |
| 6 | 167356509 | G | C | RNASET2 | p.F110L | 0 | 32 | 0.162162 | 37 | WCM131_Z2_1 | Tissue |
| 6 | 27278020 | C | A | POM121L2 | p.G644C | 0 | 153 | 0.218978 | 137 | WCM131_Z2_1 | Tissue |
| 6 | 27279632 | C | A | POM121L2 | p.W106C | 0 | 192 | 0.108108 | 148 | WCM131_Z2_1 | Tissue |
| 6 | 28297279 | C | T | ZSCAN31 | p.R61Q | 0 | 135 | 0.253846 | 130 | WCM131_Z2_1 | Tissue |
| 6 | 28964347 | C | T | ZNF311 | p.M144I | 0 | 41 | 0.113208 | 53 | WCM131_Z2_1 | Tissue |
| 6 | 30139794 | C | T | TRIM15 | p.Q356* | 0 | 67 | 0.214286 | 70 | WCM131_Z2_1 | Tissue |
| 6 | 43336946 | G | T | ZNF318 | p.P53Q | 0 | 46 | 0.130435 | 46 | WCM131_Z2_1 | Tissue |
| 6 | 46977400 | C | T | GPR110 | p.V591M | 0 | 82 | 0.2625 | 80 | WCM131_Z2_1 | Tissue |
| 6 | 49479750 | T | C | GLYATL3 | p.M16T | 0 | 109 | 0.130435 | 92 | WCM131_Z2_1 | Tissue |
| 6 | 49489397 | C | A | GLYATL3 | p.A118D | 0 | 93 | 0.136364 | 66 | WCM131_Z2_1 | Tissue |
| 6 | 52141987 | G | A | MCM3 | p.S348F | 0 | 174 | 0.240506 | 158 | WCM131_Z2_1 | Tissue |
| 6 | 5999364 | C | T | NRN1 | p.D92N | 0 | 112 | 0.15 | 120 | WCM131_Z2_1 | Tissue |
| 6 | 65300143 | G | C | EYS | p.L1873V | 0 | 74 | 0.107143 | 56 | WCM131_Z2_1 | Tissue |
| 6 | 65301217 | G | A | EYS | p.R1515W | 0 | 126 | 0.172414 | 58 | WCM131_Z2_1 | Tissue |
| 6 | 65301408 | A | G | EYS | p.I1451T | 0 | 98 | 0.116883 | 77 | WCM131_Z2_1 | Tissue |
| 6 | 65301679 | T | C | EYS | p.I1361V | 0 | 32 | 0.129032 | 31 | WCM131_Z2_1 | Tissue |
| 6 | 74183172 | C | T | MTO1 | p.T207M | 0 | 110 | 0.126437 | 87 | WCM131_Z2_1 | Tissue |
| 6 | 84896142 | C | T | KIAA1009 | p.E437K | 0 | 67 | 0.089552 | 67 | WCM131_Z2_1 | Tissue |
| 6 | 97246455 | T | C | GPR63 | p.M385V | 0 | 75 | 0.130952 | 84 | WCM131_Z2_1 | Tissue |
| 7 | 100636383 | G | A | MUC12 | p.V990I | 0 | 113 | 0.138614 | 101 | WCM131_Z2_1 | Tissue |
| 7 | 100678784 | G | T | MUC17 | p.D1363Y | 0 | 60 | 0.12069 | 58 | WCM131_Z2_1 | Tissue |
| 7 | 100678785 | A | T | MUC17 | p.D1363V | 0 | 59 | 0.12069 | 58 | WCM131_Z2_1 | Tissue |
| 7 | 116340262 | A | G | MET | p.N375S | 0 | 72 | 0.238095 | 63 | WCM131_Z2_1 | Tissue |
| 7 | 141838362 | C | G | RP11- | p.F327L | 0 | 81 | 0.268293 | 82 | WCM131_Z2_1 | Tissue |
| | | | | 1220K2.2 | | | | | | | |
| 7 | 144094613 | G | T | NOBOX | p.P599H | 0.006098 | 164 | 0.205298 | 151 | WCM131_Z2_1 | Tissue |
| 7 | 14622701 | C | T | DGKB | p.V500M | 0 | 35 | 0.069767 | 43 | WCM131_Z2_1 | Tissue |
| 7 | 150932381 | T | G | CHPF2 | p.F171V | 0 | 137 | 0.128205 | 78 | WCM131_Z2_1 | Tissue |
| 7 | 150932382 | T | C | CHPF2 | p.F171S | 0 | 137 | 0.128205 | 78 | WCM131_Z2_1 | Tissue |
| 7 | 150934469 | C | T | CHPF2 | p.R341W | 0 | 85 | 0.21978 | 91 | WCM131_Z2_1 | Tissue |
| 7 | 31692357 | C | T | CCDC129 | p.P1017S | 0 | 122 | 0.210526 | 114 | WCM131_Z2_1 | Tissue |
| 7 | 45122324 | C | A | NACAD | p.C1152F | 0 | 37 | 0.151515 | 33 | WCM131_Z2_1 | Tissue |
| 7 | 45122464 | A | C | NACAD | p.D1105E | 0 | 66 | 0.222222 | 72 | WCM131_Z2_1 | Tissue |
| 7 | 45124286 | A | G | NACAD | p.V498A | 0 | 121 | 0.222222 | 99 | WCM131_Z2_1 | Tissue |
| 7 | 48685105 | C | G | ABCA13 | p.I5058M | 0 | 178 | 0.162162 | 111 | WCM131_Z2_1 | Tissue |
| 7 | 86469006 | G | A | GRM3 | p.V726I | 0 | 62 | 0.090909 | 33 | WCM131_Z2_1 | Tissue |
| 7 | 87053289 | G | A | ABCB4 | p.T715I | 0 | 57 | 0.254545 | 55 | WCM131_Z2_1 | Tissue |
| 7 | 91779971 | T | G | LRRD1 | p.I719L | 0 | 49 | 0.14 | 50 | WCM131_Z2_1 | Tissue |
| 7 | 99145678 | C | G | FAM200A | p.R118P | 0 | 58 | 0.264151 | 53 | WCM131_Z2_1 | Tissue |
| 8 | 105511570 | C | G | LRP12 | p.K150N | 0 | 208 | 0.130631 | 222 | WCM131_Z2_1 | Tissue |
| 8 | 119945239 | G | T | TNFRSF11B | p.R111S | 0 | 121 | 0.151899 | 79 | WCM131_Z2_1 | Tissue |
| 8 | 120103453 | A | G | COLEC10 | p.K96E | 0 | 96 | 0.147287 | 129 | WCM131_Z2_1 | Tissue |
| 8 | 126194399 | A | G | NSMCE2 | p.K107E | 0 | 40 | 0.196721 | 61 | WCM131_Z2_1 | Tissue |
| 8 | 145141025 | G | T | GPAA1 | p.K621N | 0 | 87 | 0.251908 | 131 | WCM131_Z2_1 | Tissue |
| 8 | 17170887 | T | A | MTMR7 | p.M297L | 0 | 107 | 0.109375 | 128 | WCM131_Z2_1 | Tissue |
| 8 | 23147564 | G | A | R3HCC1 | p.R185K | 0 | 143 | 0.103226 | 155 | WCM131_Z2_1 | Tissue |
| 8 | 23150878 | T | G | R3HCC1 | p.L403R | 0 | 142 | 0.129496 | 139 | WCM131_Z2_1 | Tissue |
| 8 | 70476314 | A | G | SULF1 | p.Q35R | 0 | 54 | 0.138889 | 36 | WCM131_Z2_1 | Tissue |
| 9 | 100405634 | G | A | NCBP1 | p.V125M | 0 | 94 | 0.164835 | 91 | WCM131_Z2_1 | Tissue |
| 9 | 123287277 | G | A | CDK5RAP2 | p.T360I | 0 | 249 | 0.142857 | 252 | WCM131_Z2_1 | Tissue |
| 9 | 125377540 | C | T | OR1Q1 | p.P175L | 0 | 167 | 0.147826 | 115 | WCM131_Z2_1 | Tissue |
| 9 | 133305911 | G | A | HMCN2 | p.R566H | 0 | 301 | 0.147766 | 291 | WCM131_Z2_1 | Tissue |
| 9 | 134314416 | A | G | PRRC2B | p.Q146R | 0 | 78 | 0.227273 | 88 | WCM131_Z2_1 | Tissue |
| 9 | 137801702 | G | A | FCN1 | p.A308V | 0 | 110 | 0.105691 | 123 | WCM131_Z2_1 | Tissue |
| 9 | 18706950 | G | A | ADAMTSL1 | p.E594K | 0 | 167 | 0.202899 | 138 | WCM131_Z2_1 | Tissue |
| 9 | 34978079 | G | A | KIAA1045 | p.A392T | 0 | 52 | 0.25 | 72 | WCM131_Z2_1 | Tissue |
| 9 | 5358304 | T | C | PLGRKT | p.M127V | 0 | 61 | 0.1875 | 64 | WCM131_Z2_1 | Tissue |
| 9 | 80043927 | A | C | GNA14 | p.S207A | 0 | 32 | 0.176471 | 34 | WCM131_Z2_1 | Tissue |
| 9 | 96312954 | A | G | FAM120A | p.D752G | 0 | 118 | 0.185484 | 124 | WCM131_Z2_1 | Tissue |
| X | 128924982 | C | A | SASH3 | p.P123T | 0 | 39 | 0.157895 | 57 | WCM131_Z2_1 | Tissue |
| X | 152772527 | A | G | BGN | p.I265V | 0 | 42 | 0.484848 | 33 | WCM131_Z2_1 | Tissue |
| X | 19398315 | C | T | MAP3K15 | p.G838S | 0 | 118 | 0.364706 | 85 | WCM131_Z2_1 | Tissue |
| X | 27766045 | A | G | DCAF8L2 | p.T345A | 0 | 74 | 0.3625 | 80 | WCM131_Z2_1 | Tissue |
| X | 65253352 | C | T | VSIG4 | p.V126M | 0 | 104 | 0.208791 | 91 | WCM131_Z2_1 | Tissue |
| X | 8501076 | C | T | KAL1 | p.R668H | 0 | 47 | 0.0625 | 48 | WCM131_Z2_1 | Tissue |
| 15 | 43712787 | T | C | TP53BP1 | p.D1461G | 0 | 31 | 0.113208 | 53 | WCM139 | Plasma |
| 1 | 110022072 | G | T | SYPL2 | p.G241* | 0 | 86 | 0.308824 | 68 | WCM139_Z3_1 | Tissue |
| 11 | 102565721 | C | G | MMP27 | p.R337T | 0 | 160 | 0.363322 | 289 | WCM139_Z3_1 | Tissue |
| 11 | 119149311 | G | A | CBL | p.G440D | 0 | 106 | 0.368932 | 103 | WCM139_Z3_1 | Tissue |
| 11 | 17632357 | C | T | OTOG | p.A1849V | 0 | 45 | 0.468085 | 47 | WCM139_Z3_1 | Tissue |
| 1 | 158906898 | A | G | PYHIN1 | p.I66M | 0 | 56 | 0.347222 | 72 | WCM139_Z3_1 | Tissue |
| 1 | 16199609 | C | T | SPEN | p.R128C | 0 | 106 | 0.377551 | 98 | WCM139_Z3_1 | Tissue |
| 12 | 13906693 | T | G | GRIN2B | p.I190L | 0 | 43 | 0.373134 | 67 | WCM139_Z3_1 | Tissue |
| 1 | 223988456 | G | A | TP53BP2 | p.P428S | 0 | 92 | 0.247863 | 117 | WCM139_Z3_1 | Tissue |
| 12 | 28459740 | A | T | CCDC91 | p.K111N | 0 | 53 | 0.296296 | 54 | WCM139_Z3_1 | Tissue |
| 1 | 22927541 | C | G | EPHA8 | p.R897G | 0 | 76 | 0.353535 | 99 | WCM139_Z3_1 | Tissue |
| 1 | 2435432 | G | A | PLCH2 | p.A1011T | 0 | 69 | 0.333333 | 90 | WCM139_Z3_1 | Tissue |
| 12 | 50284851 | G | T | FAIM2 | p.P161T | 0 | 80 | 0.394366 | 71 | WCM139_Z3_1 | Tissue |
| 1 | 35370585 | C | T | DLGAP3 | p.D134N | 0 | 184 | 0.331776 | 214 | WCM139_Z3_1 | Tissue |
| 14 | 20216486 | G | T | OR4Q3 | p.K300N | 0 | 79 | 0.122222 | 90 | WCM139_Z3_1 | Tissue |
| 14 | 20825801 | G | A | PARP2 | p.G533R | 0 | 40 | 0.319149 | 47 | WCM139_Z3_1 | Tissue |
| 16 | 1420262 | G | A | UNKL | p.P482L | 0 | 79 | 0.058824 | 68 | WCM139_Z3_1 | Tissue |
| 16 | 4475898 | T | A | DNAJA3 | p.S6T | 0 | 131 | 0.414634 | 123 | WCM139_Z3_1 | Tissue |
| 16 | 68338031 | T | G | SLC7A6OS | p.K192N | 0 | 138 | 0.385057 | 174 | WCM139_Z3_1 | Tissue |
| 17 | 37627173 | C | G | CDK12 | p.S363* | 0 | 96 | 0.550459 | 109 | WCM139_Z3_1 | Tissue |
| 17 | 37676268 | G | A | CDK12 | p.R1008Q | 0 | 104 | 0.386598 | 194 | WCM139_Z3_1 | Tissue |
| 17 | 53848473 | A | G | PCTP | p.Y89C | 0 | 70 | 0.432432 | 74 | WCM139_Z3_1 | Tissue |
| 17 | 65919104 | G | C | BPTF | p.E2028D | 0 | 212 | 0.335878 | 131 | WCM139_Z3_1 | Tissue |
| 17 | 74383123 | G | A | SPHK1 | p.R204H | 0 | 42 | 0.315068 | 73 | WCM139_Z3_1 | Tissue |
| 18 | 74154470 | T | C | ZNF516 | p.S181G | 0 | 84 | 0.228571 | 70 | WCM139_Z3_1 | Tissue |
| 19 | 2834054 | A | G | ZNF554 | p.E274G | 0 | 76 | 0.432432 | 74 | WCM139_Z3_1 | Tissue |
| 19 | 36223914 | G | A | KMT2B | p.S2155N | 0 | 119 | 0.366667 | 120 | WCM139_Z3_1 | Tissue |
| 20 | 48522659 | G | A | SPATA2 | p.R354W | 0 | 77 | 0.308511 | 94 | WCM139_Z3_1 | Tissue |
| 21 | 30380741 | T | A | RWDD2B | p.N90I | 0 | 91 | 0.254386 | 114 | WCM139_Z3_1 | Tissue |
| 2 | 210769634 | T | C | UNC80 | p.L1510P | 0 | 49 | 0.789474 | 38 | WCM139_Z3_1 | Tissue |
| 2 | 73829384 | G | C | ALMS1 | p.V4062L | 0 | 173 | 0.323308 | 133 | WCM139_Z3_1 | Tissue |
| 3 | 180364927 | C | A | CCDC39 | p.K489N | 0 | 93 | 0.072289 | 83 | WCM139_Z3_1 | Tissue |
| 3 | 195486003 | T | A | MUC4 | p.E757V | 0 | 208 | 0.2443 | 307 | WCM139_Z3_1 | Tissue |
| 3 | 33626356 | C | T | CLASP2 | p.V710M | 0 | 62 | 0.276596 | 47 | WCM139_Z3_1 | Tissue |
| 3 | 55043418 | C | G | CACNA2D3 | p.F941L | 0 | 117 | 0.366412 | 131 | WCM139_Z3_1 | Tissue |
| 5 | 96322326 | C | G | LNPEP | p.F361L | 0 | 68 | 0.267857 | 56 | WCM139_Z3_1 | Tissue |
| 6 | 36930927 | C | T | PI16 | p.T270M | 0 | 53 | 0.247312 | 93 | WCM139_Z3_1 | Tissue |
| 7 | 114619628 | G | C | MDFIC | p.K95N | 0 | 289 | 0.290698 | 344 | WCM139_Z3_1 | Tissue |
| 8 | 88885353 | G | T | DCAF4L2 | p.Q283K | 0 | 80 | 0.417722 | 79 | WCM139_Z3_1 | Tissue |
| 8 | 97258618 | G | A | MTERFD1 | p.P248S | 0 | 40 | 0.116883 | 77 | WCM139_Z3_1 | Tissue |
| 9 | 100675731 | G | A | C9orf156 | p.R121C | 0 | 63 | 0.347826 | 69 | WCM139_Z3_1 | Tissue |
| X | 108628499 | G | A | GUCY2F | p.A990V | 0 | 44 | 0.818182 | 88 | WCM139_Z3_1 | Tissue |
| 18 | 2544212 | G | A | METTL4 | p.H419Y | 0 | 46 | 0.090909 | 99 | WCM142 | Plasma |
| 19 | 55568026 | A | G | RDH13 | p.I112T | 0 | 35 | 0.163265 | 98 | WCM142 | Plasma |
| 7 | 138793787 | G | T | ZC3HAV1 | p.N97K | 0 | 36 | 0.073171 | 41 | WCM142 | Plasma |
| 7 | 31378621 | G | A | NEUROD6 | p.R88* | 0 | 38 | 0.139785 | 93 | WCM142 | Plasma |
| 10 | 115535591 | T | A | PLEKHS1 | p.Y333N | 0 | 86 | 0.285714 | 56 | WCM14-TP1 | Plasma |
| 10 | 28884782 | C | T | WAC | p.T244M | 0 | 51 | 0.157895 | 38 | WCM14-TP1 | Plasma |
| 10 | 89692871 | G | T | PTEN | p.V119F | 0 | 73 | 0.560976 | 41 | WCM14-TP1 | Plasma |
| 1 | 115469139 | A | T | SYCP1 | p.K627N | 0 | 107 | 0.585185 | 135 | WCM14-TP1 | Plasma |
| 1 | 197019880 | G | C | F13B | p.S562C | 0 | 72 | 0.163043 | 92 | WCM14-TP1 | Plasma |
| 1 | 197020006 | G | A | F13B | p.S520F | 0 | 33 | 0.1 | 40 | WCM14-TP1 | Plasma |
| 1 | 241797444 | T | A | CHML | p.E542V | 0 | 77 | 0.323529 | 68 | WCM14-TP1 | Plasma |
| 1 | 24411081 | G | A | MYOM3 | p.T616I | 0 | 73 | 0.568965 | 58 | WCM14-TP1 | Plasma |
| 15 | 101602818 | C | T | LRRK1 | p.P1613S | 0 | 69 | 0.171429 | 35 | WCM14-TP1 | Plasma |
| 16 | 71674330 | G | A | MARVELD3 | p.M211I | 0 | 109 | 0.452381 | 42 | WCM14-TP1 | Plasma |
| 16 | 90130121 | G | A | PRDM7 | p.T136M | 0 | 76 | 0.348485 | 66 | WCM14-TP1 | Plasma |
| 19 | 44890819 | G | A | ZNF285 | p.H530Y | 0 | 71 | 0.194444 | 72 | WCM14-TP1 | Plasma |
| 20 | 45867667 | C | T | ZMYND8 | p.V814M | 0 | 110 | 0.1875 | 48 | WCM14-TP1 | Plasma |
| 2 | 206036978 | G | C | PARD3B | p.G555A | 0 | 52 | 0.192308 | 52 | WCM14-TP1 | Plasma |
| 22 | 41318352 | G | T | XPNPEP3 | p.Q357H | 0 | 79 | 0.085714 | 35 | WCM14-TP1 | Plasma |
| 3 | 185332407 | G | A | SENP2 | p.R330H | 0 | 83 | 0.193548 | 62 | WCM14-TP1 | Plasma |
| 3 | 89259166 | C | G | EPHA3 | p.R104G | 0 | 56 | 0.225 | 40 | WCM14-TP1 | Plasma |
| 4 | 46737038 | C | A | COX7B2 | p.A58S | 0 | 84 | 0.186441 | 59 | WCM14-TP1 | Plasma |
| 5 | 150075168 | G | A | RBM22 | p.R216W | 0 | 79 | 0.234375 | 64 | WCM14-TP1 | Plasma |
| 6 | 29012859 | C | T | OR2W1 | p.A32T | 0 | 69 | 0.148649 | 74 | WCM14-TP1 | Plasma |
| 6 | 70490440 | C | T | LMBRD1 | p.A85T | 0 | 80 | 0.230769 | 65 | WCM14-TP1 | Plasma |
| 7 | 111503433 | T | A | DOCK4 | p.N823I | 0 | 38 | 0.536585 | 41 | WCM14-TP1 | Plasma |
| 7 | 92244482 | T | C | CDK6 | p.Q318R | 0 | 47 | 0.108108 | 37 | WCM14-TP1 | Plasma |
| 8 | 85717033 | G | T | RALYL | p.V121F | 0 | 172 | 0.502825 | 177 | WCM14-TP1 | Plasma |
| 10 | 115535591 | T | A | PLEKHS1 | p.Y333N | 0 | 86 | 0.22973 | 74 | WCM14-TP2 | Plasma |
| 10 | 134664698 | G | A | TTC40 | p.Q1896* | 0 | 61 | 0.078947 | 38 | WCM14-TP2 | Plasma |
| 10 | 28884782 | C | T | WAC | p.T244M | 0 | 51 | 0.189189 | 74 | WCM14-TP2 | Plasma |
| 10 | 89692871 | G | T | PTEN | p.V119F | 0 | 73 | 0.44898 | 49 | WCM14-TP2 | Plasma |
| 1 | 115469139 | A | T | SYCP1 | p.K627N | 0 | 107 | 0.46729 | 107 | WCM14-TP2 | Plasma |
| 11 | 5537603 | G | T | UBQLNL | p.D23E | 0 | 40 | 0.130435 | 46 | WCM14-TP2 | Plasma |
| 11 | 62782337 | C | T | SLC22A8 | p.A32T | 0 | 41 | 0.068966 | 58 | WCM14-TP2 | Plasma |
| 1 | 197019880 | G | C | F13B | p.S562C | 0 | 72 | 0.154639 | 97 | WCM14-TP2 | Plasma |
| 1 | 197020006 | G | A | F13B | p.S520F | 0 | 33 | 0.113636 | 44 | WCM14-TP2 | Plasma |
| 1 | 197026575 | G | T | F13B | p.P276T | 0 | 52 | 0.12 | 100 | WCM14-TP2 | Plasma |
| 1 | 202287490 | C | T | LGR6 | p.R687* | 0 | 46 | 0.205882 | 34 | WCM14-TP2 | Plasma |
| 1 | 241797444 | T | A | CHML | p.E542V | 0 | 77 | 0.426829 | 82 | WCM14-TP2 | Plasma |
| 1 | 24411081 | G | A | MYOM3 | p.T616I | 0 | 73 | 0.394737 | 76 | WCM14-TP2 | Plasma |
| 15 | 101602818 | C | T | LRRK1 | p.P1613S | 0 | 69 | 0.292683 | 41 | WCM14-TP2 | Plasma |
| 15 | 50866653 | T | A | TRPM7 | p.Y1709F | 0 | 37 | 0.096774 | 31 | WCM14-TP2 | Plasma |
| 16 | 46962846 | G | T | GPT2 | p.K503N | 0 | 41 | 0.090909 | 33 | WCM14-TP2 | Plasma |
| 16 | 71674330 | G | A | MARVELD3 | p.M211I | 0 | 109 | 0.268293 | 82 | WCM14-TP2 | Plasma |
| 16 | 90130121 | G | A | PRDM7 | p.T136M | 0 | 76 | 0.253333 | 75 | WCM14-TP2 | Plasma |
| 17 | 43473626 | C | T | ARHGAP27 | p.R733H | 0 | 72 | 0.069767 | 43 | WCM14-TP2 | Plasma |
| 17 | 71418463 | C | T | SDK2 | p.D670N | 0 | 47 | 0.1875 | 32 | WCM14-TP2 | Plasma |
| 18 | 14748612 | A | T | ANKRD30B | p.N65I | 0 | 58 | 0.21875 | 32 | WCM14-TP2 | Plasma |
| 1 | 8418696 | C | T | RERE | p.R1300H | 0 | 89 | 0.26087 | 46 | WCM14-TP2 | Plasma |
| 18 | 60017090 | G | T | TNFRSF11A | p.C68F | 0 | 101 | 0.054348 | 92 | WCM14-TP2 | Plasma |
| 19 | 44890819 | G | A | ZNF285 | p.H530Y | 0 | 71 | 0.21875 | 96 | WCM14-TP2 | Plasma |
| 19 | 50045998 | G | A | RCN3 | p.D290N | 0 | 59 | 0.071429 | 42 | WCM14-TP2 | Plasma |
| 20 | 2572966 | G | A | TMC2 | p.G282D | 0 | 52 | 0.069767 | 43 | WCM14-TP2 | Plasma |
| 20 | 33971877 | G | T | UQCC1 | p.D63E | 0 | 59 | 0.061224 | 49 | WCM14-TP2 | Plasma |
| 20 | 35173396 | G | A | MYL9 | p.A37T | 0 | 55 | 0.076923 | 39 | WCM14-TP2 | Plasma |
| 20 | 45867667 | C | T | ZMYND8 | p.V814M | 0 | 110 | 0.197368 | 76 | WCM14-TP2 | Plasma |
| 2 | 206036978 | G | C | PARD3B | p.G555A | 0 | 52 | 0.183099 | 71 | WCM14-TP2 | Plasma |
| 3 | 185332407 | G | A | SENP2 | p.R330H | 0 | 83 | 0.162162 | 74 | WCM14-TP2 | Plasma |
| 3 | 89259166 | C | G | EPHA3 | p.R104G | 0 | 56 | 0.113636 | 44 | WCM14-TP2 | Plasma |
| 4 | 46737038 | C | A | COX7B2 | p.A58S | 0 | 84 | 0.15493 | 71 | WCM14-TP2 | Plasma |
| 5 | 140793488 | C | T | PCDHGA10 | p.P249L | 0 | 41 | 0.170732 | 41 | WCM14-TP2 | Plasma |
| 5 | 150075168 | G | A | RBM22 | p.R216W | 0 | 79 | 0.202703 | 74 | WCM14-TP2 | Plasma |
| 6 | 29012859 | C | T | OR2W1 | p.A32T | 0 | 69 | 0.209877 | 81 | WCM14-TP2 | Plasma |
| 6 | 47681812 | G | T | GPR115 | p.R277S | 0 | 39 | 0.411765 | 34 | WCM14-TP2 | Plasma |
| 6 | 70490440 | C | T | LMBRD1 | p.A85T | 0 | 80 | 0.168675 | 83 | WCM14-TP2 | Plasma |
| 7 | 111503433 | T | A | DOCK4 | p.N823I | 0 | 38 | 0.451613 | 62 | WCM14-TP2 | Plasma |
| 7 | 92244482 | T | C | CDK6 | p.Q318R | 0 | 47 | 0.134615 | 52 | WCM14-TP2 | Plasma |
| 8 | 145745719 | G | A | LRRC14 | p.V143I | 0 | 63 | 0.076923 | 39 | WCM14-TP2 | Plasma |
| 8 | 85717033 | G | T | RALYL | p.V121F | 0 | 172 | 0.425926 | 162 | WCM14-TP2 | Plasma |
| X | 100749082 | G | C | ARMCX4 | p.G1836R | 0 | 41 | 0.205882 | 34 | WCM14-TP2 | Plasma |
| X | 144904892 | G | T | SLITRK2 | p.V317L | 0 | 54 | 0.483871 | 31 | WCM14-TP2 | Plasma |
| X | 37265509 | A | G | PRRG1 | p.R3G | 0 | 46 | 0.064516 | 62 | WCM14-TP2 | Plasma |
| 10 | 115535591 | T | A | PLEKHS1 | p.Y333N | 0 | 86 | 0.19802 | 101 | WCM14-TP3 | Plasma |
| 10 | 28884782 | C | T | WAC | p.T244M | 0 | 51 | 0.076923 | 65 | WCM14-TP3 | Plasma |
| 10 | 89692871 | G | T | PTEN | p.V119F | 0 | 73 | 0.327273 | 55 | WCM14-TP3 | Plasma |
| 1 | 166819116 | C | A | POGK | p.R434S | 0 | 57 | 0.066667 | 45 | WCM14-TP3 | Plasma |
| 1 | 197019880 | G | C | F13B | p.S562C | 0 | 72 | 0.075472 | 106 | WCM14-TP3 | Plasma |
| 1 | 197020006 | G | A | F13B | p.S520F | 0 | 33 | 0.071429 | 56 | WCM14-TP3 | Plasma |
| 1 | 197026575 | G | T | F13B | p.P276T | 0 | 52 | 0.09434 | 106 | WCM14-TP3 | Plasma |
| 1 | 241797444 | T | A | CHML | p.E542V | 0 | 77 | 0.28972 | 107 | WCM14-TP3 | Plasma |
| 1 | 24411081 | G | A | MYOM3 | p.T616I | 0 | 73 | 0.259259 | 81 | WCM14-TP3 | Plasma |
| 1 | 29475181 | C | T | SRSF4 | p.R409H | 0 | 34 | 0.083333 | 36 | WCM14-TP3 | Plasma |
| 15 | 101602818 | C | T | LRRK1 | p.P1613S | 0 | 69 | 0.174603 | 63 | WCM14-TP3 | Plasma |
| 15 | 65425308 | T | C | PDCD7 | p.E271G | 0 | 76 | 0.151515 | 33 | WCM14-TP3 | Plasma |
| 16 | 71674330 | G | A | MARVELD3 | p.M211I | 0 | 109 | 0.190476 | 84 | WCM14-TP3 | Plasma |
| 16 | 90130121 | G | A | PRDM7 | p.T136M | 0 | 76 | 0.282353 | 85 | WCM14-TP3 | Plasma |
| 17 | 71418463 | C | T | SDK2 | p.D670N | 0 | 47 | 0.15625 | 32 | WCM14-TP3 | Plasma |
| 18 | 14748612 | A | T | ANKRD30B | p.N65I | 0 | 58 | 0.15 | 40 | WCM14-TP3 | Plasma |
| 1 | 8418696 | C | T | RERE | p.R1300H | 0 | 89 | 0.159091 | 44 | WCM14-TP3 | Plasma |
| 19 | 44890819 | G | A | ZNF285 | p.H530Y | 0 | 71 | 0.065421 | 107 | WCM14-TP3 | Plasma |
| 20 | 45867667 | C | T | ZMYND8 | p.V814M | 0 | 110 | 0.111111 | 90 | WCM14-TP3 | Plasma |
| 3 | 185332407 | G | A | SENP2 | p.R330H | 0 | 83 | 0.15534 | 103 | WCM14-TP3 | Plasma |
| 3 | 89259166 | C | G | EPHA3 | p.R104G | 0 | 56 | 0.112903 | 62 | WCM14-TP3 | Plasma |
| 4 | 46737038 | C | A | COX7B2 | p.A58S | 0 | 84 | 0.204819 | 83 | WCM14-TP3 | Plasma |
| 5 | 140793488 | C | T | PCDHGA10 | p.P249L | 0 | 41 | 0.130435 | 46 | WCM14-TP3 | Plasma |
| 5 | 150075168 | G | A | RBM22 | p.R216W | 0 | 79 | 0.11236 | 89 | WCM14-TP3 | Plasma |
| 6 | 29012859 | C | T | OR2W1 | p.A32T | 0 | 69 | 0.139785 | 93 | WCM14-TP3 | Plasma |
| 6 | 34100922 | G | T | GRM4 | p.Q118K | 0 | 42 | 0.081081 | 37 | WCM14-TP3 | Plasma |
| 6 | 47681812 | G | T | GPR115 | p.R277S | 0 | 39 | 0.323529 | 34 | WCM14-TP3 | Plasma |
| 6 | 70490440 | C | T | LMBRD1 | p.A85T | 0 | 80 | 0.144231 | 104 | WCM14-TP3 | Plasma |
| 7 | 111503433 | T | A | DOCK4 | p.N823I | 0 | 38 | 0.327869 | 61 | WCM14-TP3 | Plasma |
| X | 49075190 | G | T | CACNA1F | p.T924K | 0 | 46 | 0.071429 | 56 | WCM14-TP3 | Plasma |
| 10 | 115535591 | T | A | PLEKHS1 | p.Y333N | 0 | 86 | 0.34375 | 96 | WCM14-TP4 | Plasma |
| 10 | 28884782 | C | T | WAC | p.T244M | 0 | 51 | 0.180328 | 61 | WCM14-TP4 | Plasma |
| 10 | 89692871 | G | T | PTEN | p.V119F | 0 | 73 | 0.682927 | 41 | WCM14-TP4 | Plasma |
| 1 | 115469139 | A | T | SYCP1 | p.K627N | 0 | 107 | 0.469027 | 113 | WCM14-TP4 | Plasma |
| 11 | 40137316 | C | T | LRRC4C | p.R176Q | 0 | 40 | 0.098039 | 51 | WCM14-TP4 | Plasma |
| 1 | 197019880 | G | C | F13B | p.S562C | 0 | 72 | 0.163636 | 110 | WCM14-TP4 | Plasma |
| 1 | 197026575 | G | T | F13B | p.P276T | 0 | 52 | 0.107527 | 93 | WCM14-TP4 | Plasma |
| 1 | 202287490 | C | T | LGR6 | p.R687* | 0 | 46 | 0.142857 | 49 | WCM14-TP4 | Plasma |
| 1 | 241797444 | T | A | CHML | p.E542V | 0 | 77 | 0.488372 | 86 | WCM14-TP4 | Plasma |
| 1 | 24411081 | G | A | MYOM3 | p.T616I | 0 | 73 | 0.561798 | 89 | WCM14-TP4 | Plasma |
| 15 | 101602818 | C | T | LRRK1 | p.P1613S | 0 | 69 | 0.469697 | 66 | WCM14-TP4 | Plasma |
| 16 | 71674330 | G | A | MARVELD3 | p.M211I | 0 | 109 | 0.455556 | 90 | WCM14-TP4 | Plasma |
| 16 | 76587357 | G | A | RP11- | p.G15D | 0 | 49 | 0.08 | 50 | WCM14-TP4 | Plasma |
| | | | | 58C22.1 | | | | | | | |
| 16 | 90130121 | G | A | PRDM7 | p.T136M | 0 | 76 | 0.246914 | 81 | WCM14-TP4 | Plasma |
| 17 | 71418463 | C | T | SDK2 | p.D670N | 0 | 47 | 0.16 | 50 | WCM14-TP4 | Plasma |
| 18 | 14748612 | A | T | ANKRD30B | p.N65I | 0 | 58 | 0.179487 | 39 | WCM14-TP4 | Plasma |
| 1 | 8418696 | C | T | RERE | p.R1300H | 0 | 89 | 0.15873 | 63 | WCM14-TP4 | Plasma |
| 19 | 38573514 | G | A | SIPA1L3 | p.E437K | 0 | 36 | 0.472222 | 36 | WCM14-TP4 | Plasma |
| 19 | 44890819 | G | A | ZNF285 | p.H530Y | 0 | 71 | 0.206186 | 97 | WCM14-TP4 | Plasma |
| 20 | 45867667 | C | T | ZMYND8 | p.V814M | 0 | 110 | 0.323232 | 99 | WCM14-TP4 | Plasma |
| 2 | 206036978 | G | C | PARD3B | p.G555A | 0 | 52 | 0.202899 | 69 | WCM14-TP4 | Plasma |
| 3 | 185332407 | G | A | SENP2 | p.R330H | 0 | 83 | 0.137255 | 102 | WCM14-TP4 | Plasma |
| 3 | 89259166 | C | G | EPHA3 | p.R104G | 0 | 56 | 0.195652 | 46 | WCM14-TP4 | Plasma |
| 4 | 46737038 | C | A | COX7B2 | p.A58S | 0 | 84 | 0.142857 | 70 | WCM14-TP4 | Plasma |
| 5 | 140712178 | G | A | PCDHGA1 | p.V643M | 0 | 45 | 0.272727 | 33 | WCM14-TP4 | Plasma |
| 5 | 140793488 | C | T | PCDHGA10 | p.P249L | 0 | 41 | 0.095238 | 42 | WCM14-TP4 | Plasma |
| 5 | 150075168 | G | A | RBM22 | p.R216W | 0 | 79 | 0.157895 | 76 | WCM14-TP4 | Plasma |
| 6 | 29012859 | C | T | OR2W1 | p.A32T | 0 | 69 | 0.214286 | 98 | WCM14-TP4 | Plasma |
| 6 | 47681812 | G | T | GPR115 | p.R277S | 0 | 39 | 0.487805 | 41 | WCM14-TP4 | Plasma |
| 6 | 70490440 | C | T | LMBRD1 | p.A85T | 0 | 80 | 0.277108 | 83 | WCM14-TP4 | Plasma |
| 7 | 111503433 | T | A | DOCK4 | p.N823I | 0 | 38 | 0.571429 | 63 | WCM14-TP4 | Plasma |
| 7 | 92244482 | T | C | CDK6 | p.Q318R | 0 | 47 | 0.103448 | 58 | WCM14-TP4 | Plasma |
| 7 | 98649843 | G | C | SMURF 1 | p.L236V | 0 | 68 | 0.069307 | 101 | WCM14-TP4 | Plasma |
| 8 | 85717033 | G | T | RALYL | p.V121F | 0 | 172 | 0.666667 | 180 | WCM14-TP4 | Plasma |
| X | 37265509 | A | G | PRRG1 | p.R3G | 0 | 46 | 0.1 | 80 | WCM14-TP4 | Plasma |
| 10 | 115535591 | T | A | PLEKHS1 | p.Y333N | 0 | 87 | 0.186667 | 75 | WCM14_x1_1 | Tissue |
| 10 | 89692871 | G | T | PTEN | p.V119F | 0 | 117 | 0.49505 | 101 | WCM14_x1_1 | Tissue |
| 1 | 115469139 | A | T | SYCP1 | p.K627N | 0 | 116 | 0.348485 | 66 | WCM14_x1_1 | Tissue |
| 11 | 40137316 | C | T | LRRC4C | p.R176Q | 0 | 143 | 0.171875 | 128 | WCM14_x1_1 | Tissue |
| 11 | 5537603 | G | T | UBQLNL | p.D23E | 0 | 122 | 0.310078 | 129 | WCM14_x1_1 | Tissue |
| 1 | 241797444 | T | A | CHML | p.E542V | 0 | 177 | 0.283133 | 166 | WCM14_x1_1 | Tissue |
| 1 | 24411081 | G | A | MYOM3 | p.T616I | 0 | 103 | 0.454545 | 66 | WCM14_x1_1 | Tissue |
| 15 | 101602818 | C | T | LRRK1 | p.P1613S | 0 | 180 | 0.269608 | 204 | WCM14_x1_1 | Tissue |
| 16 | 71674330 | G | A | MARVELD3 | p.M211I | 0 | 70 | 0.402778 | 72 | WCM14_x1_1 | Tissue |
| 17 | 71418463 | C | T | SDK2 | p.D670N | 0 | 185 | 0.169697 | 165 | WCM14_x1_1 | Tissue |
| 18 | 14748612 | A | T | ANKRD30B | p.N65I | 0 | 118 | 0.204918 | 122 | WCM14_x1_1 | Tissue |
| 1 | 8418696 | C | T | RERE | p.R1300H | 0 | 133 | 0.169118 | 136 | WCM14_x1_1 | Tissue |
| 19 | 38573514 | G | A | SIPA1L3 | p.E437K | 0 | 199 | 0.274611 | 193 | WCM14_x1_1 | Tissue |
| 19 | 50251660 | G | A | TSKS | p.R156W | 0 | 95 | 0.067961 | 103 | WCM14_x1_1 | Tissue |
| 20 | 45867667 | C | T | ZMYND8 | p.V814M | 0 | 155 | 0.097744 | 133 | WCM14_x1_1 | Tissue |
| 21 | 33036109 | A | T | SOD1 | p.N27Y | 0 | 109 | 0.058824 | 102 | WCM14_x1_1 | Tissue |
| 2 | 202899559 | C | A | FZD7 | p.N63K | 0 | 98 | 0.365854 | 82 | WCM14_x1_1 | Tissue |
| 2 | 206036978 | G | C | PARD3B | p.G555A | 0 | 126 | 0.064935 | 154 | WCM14_x1_1 | Tissue |
| 3 | 185332407 | G | A | SENP2 | p.R330H | 0 | 63 | 0.060241 | 83 | WCM14_x1_1 | Tissue |
| 3 | 49689777 | C | T | BSN | p.R930C | 0 | 165 | 0.092437 | 119 | WCM14_x1_1 | Tissue |
| 4 | 46737038 | C | A | COX7B2 | p.A58S | 0 | 154 | 0.145455 | 110 | WCM14_x1_1 | Tissue |
| 5 | 140712178 | G | A | PCDHGA1 | p.V643M | 0 | 34 | 0.193548 | 31 | WCM14_x1_1 | Tissue |
| 5 | 154179454 | G | T | LARP1 | p.R446L | 0 | 167 | 0.6 | 120 | WCM14_x1_1 | Tissue |
| 6 | 110567307 | G | A | METTL24 | p.R315W | 0 | 81 | 0.134146 | 82 | WCM14_x1_1 | Tissue |
| 6 | 47681812 | G | T | GPR115 | p.R277S | 0 | 111 | 0.431193 | 109 | WCM14_x1_1 | Tissue |
| 6 | 70490440 | C | T | LMBRD1 | p.A85T | 0 | 56 | 0.326531 | 49 | WCM14_x1_1 | Tissue |
| 7 | 111503433 | T | A | DOCK4 | p.N823I | 0 | 165 | 0.451923 | 208 | WCM14_x1_1 | Tissue |
| 8 | 85717033 | G | T | RALYL | p.V121F | 0 | 59 | 0.59375 | 64 | WCM14_x1_1 | Tissue |
| 9 | 116132001 | C | T | BSPRY | p.A263V | 0 | 348 | 0.441065 | 263 | WCM14_x1_1 | Tissue |
| 9 | 140151472 | T | C | NELFB | p.F188S | 0 | 79 | 0.192308 | 52 | WCM14_x1_1 | Tissue |
| 10 | 122626092 | G | T | WDR11 | p.V336F | 0 | 62 | 0.071429 | 42 | WCM14_Z11_1 | Tissue |
| 1 | 110085766 | C | T | GPR61 | p.S41L | 0 | 43 | 0.06383 | 47 | WCM14_Z11_1 | Tissue |
| 11 | 117859166 | C | A | IL10RA | p.T46K | 0 | 45 | 0.069767 | 43 | WCM14_Z11_1 | Tissue |
| 11 | 126144826 | G | C | FOXRED1 | p.E181Q | 0 | 149 | 0.093264 | 193 | WCM14_Z11_1 | Tissue |
| 1 | 11579538 | G | T | PTCHD2 | p.L672F | 0 | 36 | 0.06383 | 47 | WCM14_Z11_1 | Tissue |
| 11 | 27395538 | C | A | LGR4 | p.G413W | 0 | 152 | 0.06383 | 94 | WCM14_Z11_1 | Tissue |
| 11 | 40137316 | C | T | LRRC4C | p.R176Q | 0 | 91 | 0.173913 | 69 | WCM14_Z11_1 | Tissue |
| 1 | 145597026 | G | T | POLR3C | p.R367S | 0 | 55 | 0.065217 | 46 | WCM14_Z11_1 | Tissue |
| 1 | 154000038 | G | T | NUP210L | p.C1297* | 0 | 71 | 0.061224 | 49 | WCM14_Z11_1 | Tissue |
| 11 | 606594 | C | A | PHRF1 | p.A536E | 0 | 51 | 0.086957 | 69 | WCM14_Z11_1 | Tissue |
| 1 | 197020006 | G | A | F13B | p.S520F | 0 | 74 | 0.139535 | 43 | WCM14_Z11_1 | Tissue |
| 1 | 197404691 | C | A | CRB1 | p.T1233N | 0 | 52 | 0.075 | 40 | WCM14_Z11_1 | Tissue |
| 1 | 202287490 | C | T | LGR6 | p.R687* | 0 | 108 | 0.338912 | 239 | WCM14_Z11_1 | Tissue |
| 1 | 211544765 | C | A | TRAF5 | p.T351K | 0 | 102 | 0.088889 | 45 | WCM14_Z11_1 | Tissue |
| 1 | 233397844 | G | T | PCNXL2 | p.R143S | 0 | 112 | 0.079208 | 101 | WCM14_Z11_1 | Tissue |
| 1 | 241797444 | T | A | CHML | p.E542V | 0 | 73 | 0.653061 | 49 | WCM14_Z11_1 | Tissue |
| 12 | 55759028 | C | A | OR6C75 | p.T45N | 0 | 45 | 0.068182 | 44 | WCM14_Z11_1 | Tissue |
| 1 | 28793176 | C | A | PHACTR4 | p.S240R | 0 | 44 | 0.085714 | 35 | WCM14_Z11_1 | Tissue |
| 13 | 32929413 | G | C | BRCA2 | p.E2475Q | 0 | 53 | 0.096774 | 31 | WCM14_Z11_1 | Tissue |
| 13 | 98642748 | C | A | IPO5 | p.A209D | 0 | 96 | 0.139535 | 43 | WCM14_Z11_1 | Tissue |
| 14 | 58975407 | G | T | KIAA0586 | p.A1420S | 0 | 64 | 0.066667 | 45 | WCM14_Z11_1 | Tissue |
| 15 | 101602818 | C | T | LRRK1 | p.P1613S | 0 | 151 | 0.32381 | 105 | WCM14_Z11_1 | Tissue |
| 15 | 28431864 | C | A | HERC2 | p.C2895F | 0 | 106 | 0.054545 | 55 | WCM14_Z11_1 | Tissue |
| 15 | 31200443 | G | T | FAN1 | p.A453S | 0.008 | 125 | 0.136364 | 44 | WCM14_Z11_1 | Tissue |
| 15 | 42529758 | C | A | TMEM87A | p.W247L | 0 | 156 | 0.068966 | 87 | WCM14_Z11_1 | Tissue |
| 15 | 43552639 | A | G | TGM5 | p.F50S | 0 | 80 | 0.133333 | 45 | WCM14_Z11_1 | Tissue |
| 15 | 62147094 | G | C | VPS13C | p.Y3712* | 0 | 60 | 0.081081 | 37 | WCM14_Z11_1 | Tissue |
| 15 | 89173466 | G | A | AEN | p.D307N | 0 | 58 | 0.0625 | 48 | WCM14_Z11_1 | Tissue |
| 15 | 91478812 | G | T | UNC45A | p.E30D | 0 | 168 | 0.061538 | 130 | WCM14_Z11_1 | Tissue |
| 1 | 6522098 | G | A | TNFRSF25 | p.P294L | 0 | 149 | 0.050847 | 177 | WCM14_Z11_1 | Tissue |
| 16 | 57261319 | G | T | RSPRY1 | p.W409C | 0 | 76 | 0.06 | 50 | WCM14_Z11_1 | Tissue |
| 16 | 71674330 | G | A | MARVELD3 | p.M211I | 0 | 133 | 0.495726 | 117 | WCM14_Z11_1 | Tissue |
| 16 | 85813414 | C | A | EMC8 | p.R178L | 0 | 73 | 0.055556 | 72 | WCM14_Z11_1 | Tissue |
| 16 | 90130121 | G | A | PRDM7 | p.T136M | 0 | 58 | 0.409091 | 66 | WCM14_Z11_1 | Tissue |
| 17 | 456623 | C | T | VPS53 | p.S595N | 0 | 36 | 0.096774 | 31 | WCM14_Z11_1 | Tissue |
| 17 | 57760498 | G | T | CLTC | p.A1306S | 0 | 66 | 0.090909 | 33 | WCM14_Z11_1 | Tissue |
| 17 | 71418463 | C | T | SDK2 | p.D670N | 0 | 257 | 0.182109 | 313 | WCM14_Z11_1 | Tissue |
| 18 | 14748612 | A | T | ANKRD30B | p.N65I | 0 | 219 | 0.209184 | 196 | WCM14_Z11_1 | Tissue |
| 18 | 25573549 | G | C | CDH2 | p.P358R | 0 | 154 | 0.063492 | 126 | WCM14_Z11_1 | Tissue |
| 18 | 32825672 | C | A | ZNF397 | p.H335N | 0 | 104 | 0.12 | 50 | WCM14_Z11_1 | Tissue |
| 19 | 1430265 | C | A | DAZAP1 | p.P259T | 0 | 33 | 0.071429 | 42 | WCM14_Z11_1 | Tissue |
| 19 | 2271949 | G | T | OAZ1 | p.D154Y | 0 | 46 | 0.073171 | 41 | WCM14_Z11_1 | Tissue |
| 19 | 38573514 | G | A | SIPA1L3 | p.E437K | 0 | 186 | 0.220588 | 204 | WCM14_Z11_1 | Tissue |
| 19 | 38774407 | G | T | SPINT2 | p.E83* | 0 | 98 | 0.070588 | 85 | WCM14_Z11_1 | Tissue |
| 1 | 94342137 | G | T | DNTTIP2 | p.Q452K | 0 | 69 | 0.081081 | 37 | WCM14_Z11_1 | Tissue |
| 19 | 44890819 | G | A | ZNF285 | p.H530Y | 0 | 74 | 0.266667 | 60 | WCM14_Z11_1 | Tissue |
| 19 | 519070 | G | A | TPGS1 | p.E174K | 0 | 80 | 0.153846 | 52 | WCM14_Z11_1 | Tissue |
| 19 | 53454264 | C | A | ZNF816 | p.R255I | 0 | 68 | 0.149254 | 67 | WCM14_Z11_1 | Tissue |
| 19 | 58232136 | G | T | ZNF671 | p.L440I | 0 | 97 | 0.097087 | 103 | WCM14_Z11_1 | Tissue |
| 2 | 113504004 | C | A | CKAP2L | p.G584V | 0 | 74 | 0.089552 | 67 | WCM14_Z11_1 | Tissue |
| 2 | 160879272 | G | T | PLA2R1 | p.H400N | 0 | 106 | 0.073171 | 82 | WCM14_Z11_1 | Tissue |
| 2 | 175202132 | C | A | SP9 | p.S440Y | 0 | 50 | 0.065574 | 61 | WCM14_Z11_1 | Tissue |
| 2 | 202899559 | C | A | FZD7 | p.N63K | 0 | 232 | 0.300412 | 243 | WCM14_Z11_1 | Tissue |
| 2 | 204304825 | G | T | RAPH1 | p.L1030I | 0 | 47 | 0.073171 | 41 | WCM14_Z11_1 | Tissue |
| 2 | 206036978 | G | C | PARD3B | p.G555A | 0 | 194 | 0.144279 | 201 | WCM14_Z11_1 | Tissue |
| 2 | 210809782 | C | A | UNC80 | p.A2287D | 0 | 102 | 0.125 | 40 | WCM14_Z11_1 | Tissue |
| 22 | 30409424 | C | T | MTMR3 | p.S466L | 0.007092 | 282 | 0.074236 | 229 | WCM14_Z11_1 | Tissue |
| 22 | 45741402 | C | A | SMC1B | p.V1182F | 0 | 38 | 0.09375 | 32 | WCM14_Z11_1 | Tissue |
| 3 | 130714923 | C | A | ATP2C1 | p.N698K | 0 | 84 | 0.061538 | 65 | WCM14_Z11_1 | Tissue |
| 3 | 13896130 | G | A | WNT7A | p.R157C | 0 | 73 | 0.083333 | 108 | WCM14_Z11_1 | Tissue |
| 3 | 142430743 | G | T | PLS1 | p.G595V | 0 | 130 | 0.054054 | 74 | WCM14_Z11_1 | Tissue |
| 3 | 185332407 | G | A | SENP2 | p.R330H | 0 | 71 | 0.19084 | 131 | WCM14_Z11_1 | Tissue |
| 4 | 183664431 | G | T | TENM3 | p.C1163F | 0 | 65 | 0.058824 | 68 | WCM14_Z11_1 | Tissue |
| 4 | 83745728 | G | T | SEC31A | p.L1131I | 0 | 69 | 0.072464 | 69 | WCM14_Z11_1 | Tissue |
| 5 | 133537696 | C | A | PPP2CA | p.R110L | 0 | 37 | 0.078947 | 38 | WCM14_Z11_1 | Tissue |
| 5 | 140712178 | G | A | PCDHGA1 | p.V643M | 0 | 60 | 0.279412 | 68 | WCM14_Z11_1 | Tissue |
| 5 | 140793488 | C | T | PCDHGA10 | p.P249L | 0 | 157 | 0.253521 | 142 | WCM14_Z11_1 | Tissue |
| 5 | 154179454 | G | T | LARP1 | p.R446L | 0.009524 | 105 | 0.770833 | 144 | WCM14_Z11_1 | Tissue |
| 6 | 110567307 | G | A | METTL24 | p.R315W | 0 | 105 | 0.216216 | 74 | WCM14_Z11_1 | Tissue |
| 6 | 170594410 | C | A | DLL1 | p.G322C | 0 | 133 | 0.074766 | 107 | WCM14_Z11_1 | Tissue |
| 6 | 170597383 | C | T | DLL1 | p.G205E | 0 | 95 | 0.102564 | 78 | WCM14_Z11_1 | Tissue |
| 6 | 34385294 | G | T | RPS10 | p.P163T | 0 | 35 | 0.09375 | 32 | WCM14_Z11_1 | Tissue |
| 6 | 47626738 | G | T | GPR111 | p.L39F | 0 | 114 | 0.088889 | 90 | WCM14_Z11_1 | Tissue |
| 6 | 47681812 | G | T | GPR115 | p.R277S | 0 | 148 | 0.456522 | 92 | WCM14_Z11_1 | Tissue |
| 6 | 70490440 | C | T | LMBRD1 | p.A85T | 0 | 53 | 0.261905 | 42 | WCM14_Z11_1 | Tissue |
| 6 | 72943521 | G | T | RIMS1 | p.R575L | 0 | 52 | 0.193548 | 31 | WCM14_Z11_1 | Tissue |
| 7 | 111503433 | T | A | DOCK4 | p.N823I | 0 | 179 | 0.40107 | 187 | WCM14_Z11_1 | Tissue |
| 7 | 122811922 | G | T | SLC13A1 | p.L89I | 0 | 61 | 0.055556 | 54 | WCM14_Z11_1 | Tissue |
| 7 | 135095294 | G | T | CNOT4 | p.N264K | 0 | 58 | 0.061224 | 49 | WCM14_Z11_1 | Tissue |
| 7 | 91870391 | C | A | KRIT1 | p.G60C | 0 | 86 | 0.057471 | 87 | WCM14_Z11_1 | Tissue |
| 7 | 92244482 | T | C | CDK6 | p.Q318R | 0 | 43 | 0.197368 | 76 | WCM14_Z11_1 | Tissue |
| 8 | 101271292 | C | T | RNF19A | p.G670E | 0 | 33 | 0.056604 | 53 | WCM14_Z11_1 | Tissue |
| 9 | 107380437 | G | T | OR13C9 | p.H17N | 0 | 45 | 0.09375 | 32 | WCM14_Z11_1 | Tissue |
| 9 | 114986244 | G | T | PTBP3 | p.L516I | 0 | 107 | 0.057143 | 70 | WCM14_Z11_1 | Tissue |
| 9 | 130210212 | G | A | RPL12 | p.R146C | 0 | 54 | 0.082192 | 73 | WCM14_Z11_1 | Tissue |
| 9 | 140151472 | T | C | NELFB | p.F188S | 0 | 180 | 0.568075 | 213 | WCM14_Z11_1 | Tissue |
| 9 | 32481395 | G | T | DDX58 | p.D527E | 0 | 58 | 0.090909 | 33 | WCM14_Z11_1 | Tissue |
| 9 | 95148521 | G | A | OGN | p.L230F | 0 | 85 | 0.055556 | 54 | WCM14_Z11_1 | Tissue |
| X | 129267363 | G | T | AIFM1 | p.A458D | 0 | 51 | 0.090909 | 33 | WCM14_Z11_1 | Tissue |
| X | 144904892 | G | T | SLITRK2 | p.V317L | 0 | 35 | 0.454545 | 44 | WCM14_Z11_1 | Tissue |
| 12 | 129190265 | C | T | TMEM132C | p.R918W | 0 | 33 | 0.102564 | 78 | WCM15 | Plasma |
| 12 | 20833108 | C | T | PDE3A | p.S1110L | 0 | 32 | 0.153846 | 52 | WCM15 | Plasma |
| 1 | 233275531 | G | T | PCNXL2 | p.Y1196* | 0 | 44 | 0.141026 | 78 | WCM15 | Plasma |
| 15 | 43748441 | A | C | TP53BP1 | p.S784A | 0 | 33 | 0.054795 | 73 | WCM15 | Plasma |
| 16 | 89245906 | G | A | CDH15 | p.R42H | 0 | 43 | 0.1625 | 80 | WCM15 | Plasma |
| 17 | 7576865 | A | C | TP53 | p.Y327* | 0 | 37 | 0.304762 | 105 | WCM15 | Plasma |
| 19 | 51021577 | C | T | LRRC4B | p.G465S | 0 | 36 | 0.234375 | 64 | WCM15 | Plasma |
| 19 | 56011593 | C | T | SSC5D | p.R706* | 0 | 36 | 0.123596 | 89 | WCM15 | Plasma |
| 3 | 66463436 | T | C | LRIG1 | p.D217G | 0 | 31 | 0.068493 | 73 | WCM15 | Plasma |
| 7 | 23213848 | T | A | KLHL7 | p.F564L | 0 | 47 | 0.225352 | 71 | WCM15 | Plasma |
| 8 | 17065500 | A | G | ZDHHC2 | p.H210R | 0 | 41 | 0.126984 | 63 | WCM15 | Plasma |
| 9 | 37745439 | C | T | FRMPD1 | p.P1137L | 0 | 33 | 0.075 | 40 | WCM15 | Plasma |
| 9 | 75406899 | G | A | TMC1 | p.W441* | 0 | 32 | 0.075472 | 53 | WCM15 | Plasma |
| 10 | 28272878 | G | T | ARMC4 | p.P238Q | 0 | 48 | 0.050633 | 79 | WCM154 | Plasma |
| 11 | 120291549 | C | T | ARHGEF12 | p.S96F | 0 | 44 | 0.191781 | 73 | WCM154 | Plasma |
| 11 | 73796788 | G | C | C2CD3 | p.S1262C | 0 | 50 | 0.189873 | 79 | WCM154 | Plasma |
| 11 | 78776035 | G | A | TENM4 | p.R81W | 0 | 42 | 0.351351 | 74 | WCM154 | Plasma |
| 1 | 201982147 | G | T | ELF3 | p.G224V | 0 | 47 | 0.202247 | 89 | WCM154 | Plasma |
| 1 | 209803163 | C | T | LAMB3 | p.E351K | 0 | 61 | 0.366337 | 101 | WCM154 | Plasma |
| 1 | 214791953 | C | T | CENPF | p.Q133* | 0 | 40 | 0.354545 | 110 | WCM154 | Plasma |
| 14 | 35565817 | T | G | PPP2R3C | p.K262T | 0 | 63 | 0.142857 | 112 | WCM154 | Plasma |
| 14 | 38061334 | G | T | FOXA1 | p.R219S | 0 | 40 | 0.28 | 75 | WCM154 | Plasma |
| 14 | 96178685 | C | T | TCL1A | p.V57I | 0 | 46 | 0.302083 | 96 | WCM154 | Plasma |
| 15 | 75108835 | G | A | LMAN1L | p.G133D | 0 | 44 | 0.060606 | 66 | WCM154 | Plasma |
| 2 | 179468729 | C | T | TTN | p.V16588M | 0 | 70 | 0.111111 | 90 | WCM154 | Plasma |
| 2 | 201485940 | G | T | AOX1 | p.E658* | 0 | 42 | 0.054795 | 73 | WCM154 | Plasma |
| 22 | 24896175 | C | G | UPB1 | p.P69A | 0 | 31 | 0.319149 | 47 | WCM154 | Plasma |
| 2 | 86707345 | C | T | KDM3A | p.S791L | 0 | 33 | 0.328767 | 73 | WCM154 | Plasma |
| 4 | 103553339 | A | G | MANBA | p.F839L | 0 | 35 | 0.2 | 60 | WCM154 | Plasma |
| 4 | 3318546 | G | A | RGS12 | p.D217N | 0 | 45 | 0.184615 | 65 | WCM154 | Plasma |
| 4 | 47954625 | G | A | CNGA1 | p.R32* | 0 | 45 | 0.206186 | 97 | WCM154 | Plasma |
| 5 | 33947380 | G | A | SLC45A2 | p.P419L | 0 | 62 | 0.325 | 120 | WCM154 | Plasma |
| 6 | 138200015 | G | T | TNFAIP3 | p.C478F | 0 | 46 | 0.384615 | 78 | WCM154 | Plasma |
| 6 | 36867354 | A | T | C6orf89 | p.K45M | 0 | 46 | 0.244898 | 98 | WCM154 | Plasma |
| 7 | 100552671 | G | T | MUC3A | p.E474D | 0 | 33 | 0.2 | 85 | WCM154 | Plasma |
| 7 | 55902249 | C | G | 14-Sep | p.D197H | 0 | 53 | 0.198113 | 106 | WCM154 | Plasma |
| 11 | 73796788 | G | C | C2CD3 | p.S1262C | 0 | 331 | 0.836653 | 251 | WCM154_Z1_1 | Tissue |
| 1 | 201982147 | G | T | ELF3 | p.G224V | 0 | 193 | 0.375 | 256 | WCM154_Z1_1 | Tissue |
| 1 | 206652374 | C | T | IKBKE | p.L361F | 0 | 91 | 0.316327 | 98 | WCM154_Z1_1 | Tissue |
| 1 | 209803163 | C | T | LAMB3 | p.E351K | 0 | 102 | 0.358209 | 134 | WCM154_Z1_1 | Tissue |
| 1 | 214791953 | C | T | CENPF | p.Q133* | 0 | 65 | 0.45 | 80 | WCM154_Z1_1 | Tissue |
| 1 | 3548141 | G | A | WRAP73 | p.R377C | 0 | 71 | 0.135135 | 37 | WCM154_Z1_1 | Tissue |
| 14 | 35565817 | T | G | PPP2R3C | p.K262T | 0 | 251 | 0.352273 | 176 | WCM154_Z1_1 | Tissue |
| 14 | 38061334 | G | T | FOXA1 | p.R219S | 0 | 32 | 0.4375 | 32 | WCM154_Z1_1 | Tissue |
| 14 | 96178685 | C | T | TCL1A | p.V57I | 0 | 76 | 0.276316 | 76 | WCM154_Z1_1 | Tissue |
| 16 | 10274172 | C | T | GRIN2A | p.A33T | 0 | 67 | 0.486842 | 76 | WCM154_Z1_1 | Tissue |
| 19 | 9047229 | C | A | MUC16 | p.G11468W | 0 | 76 | 0.180723 | 83 | WCM154_Z1_1 | Tissue |
| 21 | 31709709 | C | A | KRTAP27-1 | p.R93M | 0 | 60 | 0.27907 | 43 | WCM154_Z1_1 | Tissue |
| 2 | 204320216 | G | A | RAPH1 | p.R416C | 0 | 104 | 0.075 | 80 | WCM154_Z1_1 | Tissue |
| 2 | 86707345 | C | T | KDM3A | p.S791L | 0 | 94 | 0.85 | 80 | WCM154_Z1_1 | Tissue |
| 3 | 107491938 | G | A | BBX | p.S457N | 0 | 60 | 0.19 | 100 | WCM154_Z1_1 | Tissue |
| 3 | 153994032 | G | T | DHX36 | p.D985E | 0 | 35 | 0.085714 | 35 | WCM154_Z1_1 | Tissue |
| 3 | 19574999 | A | G | KCNH8 | p.H911R | 0 | 39 | 0.314286 | 35 | WCM154_Z1_1 | Tissue |
| 3 | 48698327 | G | T | CELSR3 | p.H581N | 0 | 57 | 0.571429 | 42 | WCM154_Z1_1 | Tissue |
| 4 | 103553339 | A | G | MANBA | p.F839L | 0 | 239 | 0.302521 | 119 | WCM154_Z1_1 | Tissue |
| 4 | 3318546 | G | A | RGS12 | p.D217N | 0 | 151 | 0.409524 | 105 | WCM154_Z1_1 | Tissue |
| 5 | 176882751 | T | G | PRR7 | p.C152G | 0 | 129 | 0.190283 | 247 | WCM154_Z1_1 | Tissue |
| 5 | 33947380 | G | A | SLC45A2 | p.P419L | 0 | 193 | 0.254902 | 357 | WCM154_Z1_1 | Tissue |
| 6 | 138200015 | G | T | TNFAIP3 | p.C478F | 0 | 79 | 0.448276 | 87 | WCM154_Z1_1 | Tissue |
| 6 | 50683084 | C | G | TFAP2D | p.Q99E | 0 | 95 | 0.295238 | 105 | WCM154_Z1_1 | Tissue |
| 7 | 140373613 | G | C | ADCK2 | p.E161D | 0 | 125 | 0.129032 | 93 | WCM154_Z1_1 | Tissue |
| 10 | 127520099 | G | T | BCCIP | p.K174N | 0 | 42 | 0.255556 | 90 | WCM155 | Plasma |
| 10 | 49667892 | C | T | ARHGAP22 | p.R165Q | 0 | 44 | 0.807229 | 83 | WCM155 | Plasma |
| 1 | 110033675 | C | T | ATXN7L2 | p.S497F | 0 | 40 | 0.260274 | 73 | WCM155 | Plasma |
| 11 | 120337925 | G | A | ARHGEF12 | p.C921Y | 0 | 53 | 0.221154 | 104 | WCM155 | Plasma |
| 11 | 20078159 | G | A | NAV2 | p.A1662T | 0 | 35 | 0.148649 | 74 | WCM155 | Plasma |
| 12 | 131466566 | C | T | GPR133 | p.R150W | 0 | 32 | 0.810345 | 58 | WCM155 | Plasma |
| 12 | 41966358 | G | A | PDZRN4 | p.D593N | 0 | 33 | 0.394231 | 104 | WCM155 | Plasma |
| 14 | 63860559 | G | C | PPP2R5E | p.S276R | 0 | 34 | 0.195652 | 92 | WCM155 | Plasma |
| 16 | 76523715 | G | A | CNTNAP4 | p.R675H | 0 | 41 | 0.78481 | 79 | WCM155 | Plasma |
| 19 | 41072213 | A | G | SPTBN4 | p.K2095R | 0 | 55 | 0.055556 | 126 | WCM155 | Plasma |
| 19 | 41072216 | C | G | SPTBN4 | p.A2096G | 0 | 59 | 0.054688 | 128 | WCM155 | Plasma |
| 19 | 51020884 | C | T | LRRC4B | p.E696K | 0 | 42 | 0.318182 | 66 | WCM155 | Plasma |
| 2 | 160082208 | C | T | TANC1 | p.A1274V | 0 | 55 | 0.377778 | 90 | WCM155 | Plasma |
| 2 | 179535859 | G | T | TTN | p.P11382T | 0 | 73 | 0.295082 | 122 | WCM155 | Plasma |
| 2 | 202025453 | G | C | CFLAR | p.E364D | 0 | 40 | 0.283951 | 81 | WCM155 | Plasma |
| 3 | 100275801 | G | A | TMEM45A | p.A130T | 0 | 37 | 0.291262 | 103 | WCM155 | Plasma |
| 3 | 14183242 | C | T | TMEM43 | p.L384F | 0 | 47 | 0.169492 | 118 | WCM155 | Plasma |
| 5 | 149263008 | G | A | PDE6A | p.R707W | 0 | 43 | 0.325 | 120 | WCM155 | Plasma |
| 5 | 66406010 | C | T | MAST4 | p.R481W | 0 | 58 | 0.211268 | 142 | WCM155 | Plasma |
| 7 | 150439339 | T | G | GIMAP5 | p.C38G | 0 | 33 | 0.147368 | 95 | WCM155 | Plasma |
| 10 | 127520099 | G | T | BCCIP | p.K174N | 0 | 149 | 0.366013 | 153 | WCM155_Z1_1 | Tissue |
| 10 | 49667892 | C | T | ARHGAP22 | p.R165Q | 0 | 211 | 1 | 129 | WCM155_Z1_1 | Tissue |
| 1 | 110033675 | C | T | ATXN7L2 | p.S497F | 0 | 150 | 0.066667 | 180 | WCM155_Z1_1 | Tissue |
| 1 | 115282345 | T | C | CSDE1 | p.Y56C | 0 | 98 | 0.273438 | 128 | WCM155_Z1_1 | Tissue |
| 11 | 20078159 | G | A | NAV2 | p.A1662T | 0 | 107 | 0.388889 | 108 | WCM155_Z1_1 | Tissue |
| 12 | 131466566 | C | T | GPR133 | p.R150W | 0 | 51 | 0.979592 | 49 | WCM155_Z1_1 | Tissue |
| 1 | 216737678 | T | C | ESRRG | p.I249V | 0 | 169 | 0.280193 | 207 | WCM155_Z1_1 | Tissue |
| 12 | 41966358 | G | A | PDZRN4 | p.D593N | 0 | 223 | 0.508816 | 397 | WCM155_Z1_1 | Tissue |
| 1 | 40703445 | A | G | RLF | p.D1024G | 0 | 136 | 0.201754 | 228 | WCM155_Z1_1 | Tissue |
| 14 | 38218349 | G | A | TTC6 | p.V857I | 0 | 123 | 0.680556 | 216 | WCM155_Z1_1 | Tissue |
| 15 | 42146271 | G | T | SPTBN5 | p.Q3251K | 0 | 173 | 0.075 | 80 | WCM155_Z1_1 | Tissue |
| 17 | 10532978 | G | A | MYH3 | p.A1911V | 0 | 60 | 0.594937 | 79 | WCM155_Z1_1 | Tissue |
| 19 | 2245540 | G | A | SF3A2 | p.R114H | 0 | 122 | 0.068966 | 116 | WCM155_Z1_1 | Tissue |
| 19 | 51020884 | C | T | LRRC4B | p.E696K | 0 | 138 | 0.439024 | 123 | WCM155_Z1_1 | Tissue |
| 19 | 9088753 | A | T | MUC16 | p.I1021N | 0 | 192 | 0.62439 | 205 | WCM155_Z1_1 | Tissue |
| 20 | 31291834 | C | A | COMMD7 | p.V173L | 0 | 75 | 0.370787 | 89 | WCM155_Z1_1 | Tissue |
| 20 | 47265971 | C | G | PREX1 | p.G1058R | 0 | 50 | 0.183673 | 49 | WCM155_Z1_1 | Tissue |
| 2 | 141707904 | T | A | LRP1B | p.R1012S | 0 | 155 | 0.333333 | 87 | WCM155_Z1_1 | Tissue |
| 2 | 241680699 | C | A | KIF1A | p.E1145* | 0 | 154 | 0.52381 | 84 | WCM155_Z1_1 | Tissue |
| 3 | 148880630 | C | T | HPS3 | p.P816S | 0 | 52 | 0.5 | 40 | WCM155_Z1_1 | Tissue |
| 3 | 9953155 | G | T | IL17RE | p.M367I | 0 | 68 | 0.061224 | 49 | WCM155_Z1_1 | Tissue |
| 5 | 149263008 | G | A | PDE6A | p.R707W | 0 | 153 | 0.341615 | 161 | WCM155_Z1_1 | Tissue |
| 5 | 66406010 | C | T | MAST4 | p.R481W | 0 | 80 | 0.313953 | 86 | WCM155_Z1_1 | Tissue |
| 7 | 150439339 | T | G | GIMAP5 | p.C38G | 0 | 231 | 0.195946 | 296 | WCM155_Z1_1 | Tissue |
| 7 | 31378317 | A | G | NEUROD6 | p.L189P | 0 | 88 | 0.315068 | 73 | WCM155_Z1_1 | Tissue |
| 7 | 6465708 | C | A | DAGLB | p.G323C | 0 | 65 | 0.095238 | 63 | WCM155_Z1_1 | Tissue |
| 8 | 146002914 | C | T | ZNF34 | p.R91Q | 0 | 101 | 0.547826 | 115 | WCM155_Z1_1 | Tissue |
| X | 110492205 | C | A | CAPN6 | p.M413I | 0 | 55 | 0.4375 | 48 | WCM155_Z1_1 | Tissue |
| 10 | 127520099 | G | T | BCCIP | p.K174N | 0 | 149 | 0.227758 | 281 | WCM155_Z2_1 | Tissue |
| 10 | 49667892 | C | T | ARHGAP22 | p.R165Q | 0 | 211 | 0.771739 | 184 | WCM155_Z2_1 | Tissue |
| 11 | 100558491 | G | T | ARHGAP42 | p.E28* | 0 | 44 | 0.083333 | 36 | WCM155_Z2_1 | Tissue |
| 11 | 111590677 | G | A | SIK2 | p.R482Q | 0 | 49 | 0.096774 | 62 | WCM155_Z2_1 | Tissue |
| 1 | 115282345 | T | C | CSDE1 | p.Y56C | 0 | 98 | 0.236641 | 131 | WCM155_Z2_1 | Tissue |
| 11 | 20078159 | G | A | NAV2 | p.A1662T | 0 | 107 | 0.134146 | 82 | WCM155_Z2_1 | Tissue |
| 11 | 56345191 | A | G | OR5M10 | p.S3P | 0 | 53 | 0.369565 | 46 | WCM155_Z2_1 | Tissue |
| 1 | 216737678 | T | C | ESRRG | p.I249V | 0 | 169 | 0.390244 | 246 | WCM155_Z2_1 | Tissue |
| 12 | 41966358 | G | A | PDZRN4 | p.D593N | 0 | 223 | 0.443662 | 568 | WCM155_Z2_1 | Tissue |
| 12 | 6701583 | C | T | CHD4 | p.R975H | 0 | 31 | 0.080645 | 62 | WCM155_Z2_1 | Tissue |
| 1 | 40703445 | A | G | RLF | p.D1024G | 0 | 136 | 0.250784 | 319 | WCM155_Z2_1 | Tissue |
| 14 | 38218349 | G | A | TTC6 | p.V857I | 0 | 123 | 0.47541 | 61 | WCM155_Z2_1 | Tissue |
| 16 | 1476220 | T | C | C16orf91 | p.R135G | 0 | 59 | 0.070175 | 57 | WCM155_Z2_1 | Tissue |
| 16 | 27788943 | G | A | KIAA0556 | p.D1522N | 0 | 91 | 0.081633 | 49 | WCM155_Z2_1 | Tissue |
| 16 | 31504801 | G | A | C16orf58 | p.R335C | 0 | 74 | 0.052632 | 152 | WCM155_Z2_1 | Tissue |
| 17 | 10532978 | G | A | MYH3 | p.A1911V | 0 | 60 | 0.444444 | 126 | WCM155_Z2_1 | Tissue |
| 17 | 41166132 | C | T | IFI35 | p.S226L | 0 | 41 | 0.363636 | 44 | WCM155_Z2_1 | Tissue |
| 18 | 2599039 | T | C | NDC80 | p.Y415H | 0 | 66 | 0.555556 | 72 | WCM155_Z2_1 | Tissue |
| 19 | 51020884 | C | T | LRRC4B | p.E696K | 0 | 138 | 0.242236 | 161 | WCM155_Z2_1 | Tissue |
| 19 | 9088753 | A | T | MUC16 | p.I1021N | 0 | 192 | 0.513089 | 191 | WCM155_Z2_1 | Tissue |
| 19 | 9089011 | G | C | MUC16 | p.A935G | 0 | 94 | 0.52381 | 63 | WCM155_Z2_1 | Tissue |
| 20 | 31291834 | C | A | COMMD7 | p.V173L | 0 | 75 | 0.245614 | 57 | WCM155_Z2_1 | Tissue |
| 2 | 141707904 | T | A | LRP1B | p.R1012S | 0 | 155 | 0.22335 | 197 | WCM155_Z2_1 | Tissue |
| 2 | 160082208 | C | T | TANC1 | p.A1274V | 0 | 167 | 0.553957 | 139 | WCM155_Z2_1 | Tissue |
| 2 | 166740465 | G | A | TTC21B | p.R1175* | 0 | 47 | 0.103448 | 58 | WCM155_Z2_1 | Tissue |
| 22 | 40055853 | C | T | CACNA1I | p.A867V | 0 | 141 | 0.105263 | 76 | WCM155_Z2_1 | Tissue |
| 2 | 241680699 | C | A | KIF1A | p.E1145* | 0 | 154 | 0.26087 | 92 | WCM155_Z2_1 | Tissue |
| 3 | 100275801 | G | A | TMEM45A | p.A130T | 0 | 69 | 0.242857 | 70 | WCM155_Z2_1 | Tissue |
| 3 | 179371120 | G | T | USP13 | p.R36L | 0 | 33 | 0.067416 | 89 | WCM155_Z2_1 | Tissue |
| 4 | 71554576 | C | A | UTP3 | p.A61D | 0 | 45 | 0.086207 | 58 | WCM155_Z2_1 | Tissue |
| 5 | 149263008 | G | A | PDE6A | p.R707W | 0 | 153 | 0.289017 | 173 | WCM155_Z2_1 | Tissue |
| 5 | 161115966 | G | A | GABRA6 | p.M79I | 0 | 169 | 0.248705 | 193 | WCM155_Z2_1 | Tissue |
| 5 | 66406010 | C | T | MAST4 | p.R481W | 0 | 80 | 0.166667 | 72 | WCM155_Z2_1 | Tissue |
| 7 | 150439339 | T | G | GIMAP5 | p.C38G | 0 | 231 | 0.284672 | 411 | WCM155_Z2_1 | Tissue |
| 7 | 31378317 | A | G | NEUROD6 | p.L189P | 0 | 88 | 0.16129 | 93 | WCM155_Z2_1 | Tissue |
| 8 | 146002914 | C | T | ZNF34 | p.R91Q | 0 | 101 | 0.314685 | 143 | WCM155_Z2_1 | Tissue |
| X | 110492205 | C | A | CAPN6 | p.M413I | 0 | 55 | 0.28169 | 71 | WCM155_Z2_1 | Tissue |
| 11 | 65984211 | C | T | PACS1 | p.R315W | 0 | 31 | 0.136364 | 66 | WCM156 | Plasma |
| 11 | 7960298 | A | C | OR10A3 | p.M257R | 0 | 32 | 0.084507 | 71 | WCM156 | Plasma |
| 22 | 24898163 | T | A | UPB1 | p.C116S | 0 | 33 | 0.108696 | 46 | WCM156 | Plasma |
| 2 | 64682532 | G | A | LGALSL | p.V31M | 0 | 34 | 0.071429 | 42 | WCM156 | Plasma |
| 6 | 32183057 | G | A | NOTCH4 | p.P656L | 0 | 31 | 0.06 | 50 | WCM156 | Plasma |
| 6 | 74063875 | A | T | DPPA5 | p.V25E | 0 | 31 | 0.056338 | 71 | WCM156 | Plasma |
| 10 | 47000144 | C | A | GPRIN2 | p.P422T | 0 | 204 | 0.144186 | 215 | WCM156_Z5_1 | Tissue |
| 10 | 61835487 | C | T | ANK3 | p.G1718R | 0 | 108 | 0.320513 | 78 | WCM156_Z5_1 | Tissue |
| 11 | 17741636 | C | T | MYOD1 | p.R103C | 0 | 57 | 0.2 | 40 | WCM156_Z5_1 | Tissue |
| 1 | 1268053 | T | G | TAS1R3 | p.V381G | 0 | 63 | 0.364865 | 74 | WCM156_Z5_1 | Tissue |
| 11 | 46916329 | G | A | LRP4 | p.R451W | 0 | 94 | 0.159574 | 94 | WCM156_Z5_1 | Tissue |
| 11 | 76255526 | A | T | C11orf30 | p.Q978L | 0 | 67 | 0.268293 | 41 | WCM156_Z5_1 | Tissue |
| 11 | 7960298 | A | C | OR10A3 | p.M257R | 0 | 95 | 0.1875 | 112 | WCM156_Z5_1 | Tissue |
| 1 | 235972382 | T | C | LYST | p.N579S | 0 | 147 | 0.19209 | 177 | WCM156_Z5_1 | Tissue |
| 12 | 57858962 | C | A | GLI1 | p.P153H | 0 | 154 | 0.051813 | 193 | WCM156_Z5_1 | Tissue |
| 12 | 95927279 | C | T | USP44 | p.E252K | 0 | 34 | 0.255814 | 43 | WCM156_Z5_1 | Tissue |
| 13 | 46718638 | G | A | LCP1 | p.R398W | 0 | 90 | 0.092308 | 65 | WCM156_Z5_1 | Tissue |
| 15 | 89386693 | G | C | ACAN | p.A289P | 0 | 40 | 0.226415 | 53 | WCM156_Z5_1 | Tissue |
| 16 | 31388172 | G | T | ITGAX | p.G854V | 0 | 178 | 0.20904 | 177 | WCM156_Z5_1 | Tissue |
| 16 | 58314519 | G | C | PRSS54 | p.S266C | 0 | 188 | 0.143713 | 167 | WCM156_Z5_1 | Tissue |
| 17 | 26902206 | T | G | ALDOC | p.K87Q | 0 | 204 | 0.263959 | 197 | WCM156_Z5_1 | Tissue |
| 17 | 51063816 | G | T | C17orf112 | p.E20D | 0 | 134 | 0.120482 | 166 | WCM156_Z5_1 | Tissue |
| 17 | 7247261 | G | C | ACAP1 | p.E219Q | 0 | 184 | 0.291667 | 144 | WCM156_Z5_1 | Tissue |
| 17 | 7577505 | T | G | TP53 | p.D259A | 0 | 127 | 0.346457 | 127 | WCM156_Z5_1 | Tissue |
| 1 | 7880612 | C | G | PER3 | p.I615M | 0 | 65 | 0.418182 | 55 | WCM156_Z5_1 | Tissue |
| 18 | 50994287 | A | T | DCC | p.S1215C | 0 | 197 | 0.308824 | 204 | WCM156_Z5_1 | Tissue |
| 19 | 36581409 | G | A | WDR62 | p.G674E | 0 | 112 | 0.11 | 100 | WCM156_Z5_1 | Tissue |
| 19 | 37368472 | G | A | ZNF345 | p.R247Q | 0 | 72 | 0.188679 | 53 | WCM156_Z5_1 | Tissue |
| 19 | 41008803 | A | T | SPTBN4 | p.E442V | 0 | 127 | 0.256198 | 121 | WCM156_Z5_1 | Tissue |
| 19 | 46974024 | C | A | PNMAL1 | p.G90V | 0 | 200 | 0.297468 | 158 | WCM156_Z5_1 | Tissue |
| 19 | 51560004 | T | G | KLK13 | p.N225T | 0 | 171 | 0.195652 | 138 | WCM156_Z5_1 | Tissue |
| 19 | 56090128 | G | T | ZNF579 | p.S293Y | 0 | 54 | 0.115385 | 52 | WCM156_Z5_1 | Tissue |
| 19 | 57985004 | T | C | ZNF772 | p.S370G | 0 | 128 | 0.287879 | 198 | WCM156_Z5_1 | Tissue |
| 20 | 3673226 | G | T | SIGLEC1 | p.C1324* | 0 | 130 | 0.26087 | 92 | WCM156_Z5_1 | Tissue |
| 21 | 30699104 | T | A | BACH1 | p.L320H | 0 | 91 | 0.290323 | 93 | WCM156_Z5_1 | Tissue |
| 2 | 191862655 | C | G | STAT1 | p.E238Q | 0 | 68 | 0.3 | 80 | WCM156_Z5_1 | Tissue |
| 22 | 24898163 | T | A | UPB1 | p.C116S | 0 | 110 | 0.234043 | 94 | WCM156_Z5_1 | Tissue |
| 22 | 30769717 | G | T | CCDC157 | p.E489D | 0 | 106 | 0.073394 | 109 | WCM156_Z5_1 | Tissue |
| 2 | 232137734 | A | G | ARMC9 | p.D426G | 0 | 239 | 0.169811 | 265 | WCM156_Z5_1 | Tissue |
| 22 | 41077031 | G | T | MCHR1 | p.G123V | 0 | 107 | 0.283951 | 81 | WCM156_Z5_1 | Tissue |
| 2 | 27447269 | G | A | CAD | p.G389R | 0 | 161 | 0.291005 | 189 | WCM156_Z5_1 | Tissue |
| 3 | 11059028 | G | A | SLC6A1 | p.R44Q | 0 | 121 | 0.256944 | 144 | WCM156_Z5_1 | Tissue |
| 3 | 170716017 | T | A | SLC2A2 | p.N447Y | 0 | 37 | 0.217391 | 46 | WCM156_Z5_1 | Tissue |
| 3 | 180337072 | A | G | CCDC39 | p.I747T | 0 | 67 | 0.27027 | 37 | WCM156_Z5_1 | Tissue |
| 3 | 196541356 | G | A | PAK2 | p.E324K | 0 | 104 | 0.289474 | 76 | WCM156_Z5_1 | Tissue |
| 3 | 4718403 | A | C | ITPR1 | p.E947A | 0 | 129 | 0.119658 | 117 | WCM156_Z5_1 | Tissue |
| 3 | 69134338 | A | C | ARL6IP5 | p.Y47S | 0 | 48 | 0.410256 | 39 | WCM156_Z5_1 | Tissue |
| 4 | 123164219 | T | G | KIAA1109 | p.S1580A | 0 | 119 | 0.163265 | 98 | WCM156_Z5_1 | Tissue |
| 4 | 55593428 | T | A | KIT | p.F529I | 0 | 81 | 0.264368 | 87 | WCM156_Z5_1 | Tissue |
| 4 | 8229765 | C | G | SH3TC1 | p.Q782E | 0 | 51 | 0.169014 | 71 | WCM156_Z5_1 | Tissue |
| 7 | 103205909 | A | G | RELN | p.F1676L | 0 | 69 | 0.223684 | 76 | WCM156_Z5_1 | Tissue |
| 7 | 150439456 | A | C | GIMAP5 | p.K77Q | 0 | 87 | 0.158273 | 139 | WCM156_Z5_1 | Tissue |
| 7 | 36450221 | A | T | ANLN | p.I399F | 0 | 130 | 0.261261 | 111 | WCM156_Z5_1 | Tissue |
| 8 | 121209117 | G | T | COL14A1 | p.R175I | 0 | 105 | 0.152778 | 144 | WCM156_Z5_1 | Tissue |
| 8 | 88218228 | C | G | CNBD1 | p.H147D | 0 | 139 | 0.216 | 125 | WCM156_Z5_1 | Tissue |
| 8 | 94146540 | G | A | C8orf87 | p.Q97* | 0 | 84 | 0.169231 | 130 | WCM156_Z5_1 | Tissue |
| 9 | 139730224 | A | C | RABL6 | p.N246H | 0 | 141 | 0.194286 | 175 | WCM156_Z5_1 | Tissue |
| 9 | 71395309 | C | A | FAM122A | p.P77T | 0 | 43 | 0.078947 | 38 | WCM156_Z5_1 | Tissue |
| 9 | 71628527 | A | T | PRKACG | p.L161H | 0 | 79 | 0.148936 | 94 | WCM156_Z5_1 | Tissue |
| 9 | 90343258 | C | T | CTSL | p.P115S | 0 | 64 | 0.296296 | 54 | WCM156_Z5_1 | Tissue |
| X | 40514221 | G | C | MED14 | p.P1355R | 0 | 51 | 0.227273 | 44 | WCM156_Z5_1 | Tissue |
| 12 | 132325175 | G | T | MMP17 | p.M160I | 0 | 32 | 0.050633 | 79 | WCM158 | Plasma |
| 15 | 43749223 | C | G | TP53BP1 | p.S523T | 0 | 58 | 0.060241 | 83 | WCM158 | Plasma |
| 15 | 43749224 | T | C | TP53BP1 | p.S523G | 0 | 59 | 0.059524 | 84 | WCM158 | Plasma |
| 17 | 40706506 | C | T | HSD17B1 | p.T208M | 0 | 42 | 0.071429 | 56 | WCM158 | Plasma |
| 17 | 4098298 | G | T | ANKFY1 | p.H449Q | 0 | 31 | 0.054795 | 73 | WCM158 | Plasma |
| 2 | 234581202 | C | T | UGT1A9 | p.R208W | 0 | 40 | 0.078431 | 51 | WCM158 | Plasma |
| 9 | 130540902 | G | T | SH2D3C | p.T2K | 0 | 36 | 0.060606 | 66 | WCM158 | Plasma |
| X | 37027959 | C | G | FAM47C | p.D492E | 0 | 39 | 0.054795 | 73 | WCM158 | Plasma |
| 1 | 103404628 | G | A | COL11A1 | p.P1134L | 0 | 76 | 0.169811 | 53 | WCM158_X1_1 | Tissue |
| 11 | 1993910 | T | G | MRPL23 | p.V140G | 0 | 163 | 0.238095 | 189 | WCM158_X1_1 | Tissue |
| 1 | 153516257 | T | C | S100A4 | p.D95G | 0 | 200 | 0.205882 | 238 | WCM158_X1_1 | Tissue |
| 11 | 6789770 | A | G | OR2AG2 | p.V140A | 0 | 126 | 0.196429 | 112 | WCM158_X1_1 | Tissue |
| 1 | 181701707 | C | T | CACNA1E | p.R829* | 0 | 82 | 0.086207 | 116 | WCM158_X1_1 | Tissue |
| 1 | 182500016 | A | G | RGSL1 | p.I779V | 0 | 93 | 0.284615 | 130 | WCM158_X1_1 | Tissue |
| 1 | 186934669 | C | T | PLA2G4A | p.L570F | 0 | 118 | 0.141593 | 113 | WCM158_X1_1 | Tissue |
| 11 | 94349694 | C | A | PIWIL4 | p.T660N | 0 | 42 | 0.08 | 50 | WCM158_X1_1 | Tissue |
| 1 | 217783692 | T | A | GPATCH2 | p.K357* | 0 | 72 | 0.346154 | 78 | WCM158_X1_1 | Tissue |
| 13 | 101266669 | G | T | TMTC4 | p.R599S | 0 | 66 | 0.071429 | 42 | WCM158_X1_1 | Tissue |
| 1 | 45271330 | C | T | PLK3 | p.R641W | 0 | 91 | 0.325843 | 89 | WCM158_X1_1 | Tissue |
| 14 | 65246451 | C | G | SPTB | p.D1489H | 0 | 46 | 0.392157 | 51 | WCM158_X1_1 | Tissue |
| 15 | 63008544 | C | A | TLN2 | p.N883K | 0 | 46 | 0.096774 | 31 | WCM158_X1_1 | Tissue |
| 16 | 602629 | C | T | CAPN15 | p.P921L | 0 | 40 | 0.25 | 32 | WCM158_X1_1 | Tissue |
| 16 | 61687934 | C | T | CDH8 | p.V660I | 0 | 92 | 0.32 | 50 | WCM158_X1_1 | Tissue |
| 17 | 39182914 | C | T | KRTAP1-5 | p.R165H | 0 | 179 | 0.202899 | 207 | WCM158_X1_1 | Tissue |
| 17 | 47696426 | A | C | SPOP | p.F133V | 0 | 130 | 0.226415 | 106 | WCM158_X1_1 | Tissue |
| 17 | 48941097 | G | C | TOB1 | p.I94M | 0 | 36 | 0.242424 | 33 | WCM158_X1_1 | Tissue |
| 17 | 73488775 | C | T | KIAA0195 | p.S606F | 0 | 186 | 0.221519 | 158 | WCM158_X1_1 | Tissue |
| 17 | 8408097 | T | G | MYH10 | p.K1141Q | 0 | 167 | 0.416 | 125 | WCM158_X1_1 | Tissue |
| 19 | 20117462 | T | G | ZNF682 | p.K283N | 0 | 69 | 0.160714 | 56 | WCM158_X1_1 | Tissue |
| 19 | 2767654 | C | A | SGTA | p.G44V | 0 | 64 | 0.241379 | 58 | WCM158_X1_1 | Tissue |
| 20 | 4771204 | G | A | RASSF2 | p.R144C | 0 | 41 | 0.159091 | 44 | WCM158_X1_1 | Tissue |
| 2 | 110373058 | C | T | SOWAHC | p.P331L | 0 | 110 | 0.06383 | 94 | WCM158_X1_1 | Tissue |
| 3 | 101086714 | G | A | SENP7 | p.S313F | 0 | 70 | 0.52439 | 82 | WCM158_X1_1 | Tissue |
| 3 | 131101026 | G | A | NUDT16 | p.R92H | 0 | 95 | 0.244898 | 98 | WCM158_X1_1 | Tissue |
| 3 | 64589750 | C | G | ADAMTS9 | p.G1199R | 0 | 57 | 0.081967 | 61 | WCM158_X1_1 | Tissue |
| 5 | 140552589 | A | G | PCDHB7 | p.E58G | 0 | 128 | 0.290323 | 93 | WCM158_X1_1 | Tissue |
| 5 | 176894484 | C | T | DBN1 | p.V159M | 0 | 103 | 0.21978 | 91 | WCM158_X1_1 | Tissue |
| 6 | 43306314 | C | A | ZNF318 | p.D1808Y | 0 | 133 | 0.103774 | 106 | WCM158_X1_1 | Tissue |
| 7 | 5427854 | C | A | TNRC18 | p.R534L | 0 | 60 | 0.1 | 60 | WCM158_X1_1 | Tissue |
| 8 | 36661568 | C | A | KCNU1 | p.S113R | 0 | 40 | 0.078947 | 38 | WCM158_X1_1 | Tissue |
| 9 | 116151886 | C | T | ALAD | p.R236Q | 0 | 62 | 0.304348 | 92 | WCM158_X1_1 | Tissue |
| X | 127186074 | C | T | ACTRT1 | p.V38I | 0 | 47 | 0.225806 | 31 | WCM158_X1_1 | Tissue |
| X | 1409310 | G | T | CSF2RA | p.G185V | 0 | 47 | 0.065217 | 46 | WCM158_X1_1 | Tissue |
| 1 | 109649197 | G | T | C1orf194 | p.N133K | 0 | 34 | 0.157895 | 38 | WCM15_Z1_1 | Tissue |
| 11 | 59811056 | A | T | PLAC1L | p.N60I | 0 | 62 | 0.145161 | 62 | WCM15_Z1_1 | Tissue |
| 1 | 166039635 | C | T | FAM78B | p.R210Q | 0 | 216 | 0.450199 | 251 | WCM15_Z1_1 | Tissue |
| 1 | 181702034 | C | T | CACNA1E | p.R938W | 0 | 173 | 0.42 | 200 | WCM15_Z1_1 | Tissue |
| 12 | 121947572 | T | A | KDM2B | p.K482M | 0 | 131 | 0.122093 | 172 | WCM15_Z1_1 | Tissue |
| 12 | 22005053 | C | T | ABCC9 | p.R916Q | 0 | 62 | 0.493151 | 73 | WCM15_Z1_1 | Tissue |
| 1 | 22332229 | C | T | CELA3A | p.P101L | 0 | 145 | 0.138158 | 152 | WCM15_Z1_1 | Tissue |
| 1 | 233275531 | G | T | PCNXL2 | p.Y1196* | 0 | 104 | 0.481132 | 106 | WCM15_Z1_1 | Tissue |
| 1 | 245019208 | C | T | HNRNPU | p.G722E | 0 | 41 | 0.491525 | 59 | WCM15_Z1_1 | Tissue |
| 12 | 57437688 | C | A | MYO1A | p.E282* | 0 | 342 | 0.488722 | 399 | WCM15_Z1_1 | Tissue |
| 13 | 61986159 | C | A | PCDH20 | p.K691N | 0 | 321 | 0.058511 | 188 | WCM15_Z1_1 | Tissue |
| 15 | 62232878 | T | A | VPS13C | p.M1857L | 0 | 117 | 0.150943 | 106 | WCM15_Z1_1 | Tissue |
| 17 | 3661050 | T | C | ITGAE | p.T324A | 0 | 91 | 0.127273 | 110 | WCM15_Z1_1 | Tissue |
| 17 | 7576865 | A | C | TP53 | p.Y327* | 0 | 131 | 0.965 | 200 | WCM15_Z1_1 | Tissue |
| 19 | 1119989 | C | T | SBNO2 | p.A395T | 0 | 144 | 0.5 | 112 | WCM15_Z1_1 | Tissue |
| 19 | 2272982 | C | T | OAZ1 | p.R188* | 0 | 200 | 0.412698 | 189 | WCM15_Z1_1 | Tissue |
| 19 | 31039968 | C | A | ZNF536 | p.L1148M | 0 | 193 | 0.082781 | 302 | WCM15_Z1_1 | Tissue |
| 19 | 36268761 | C | T | ARHGAP33 | p.A47V | 0 | 160 | 0.297619 | 336 | WCM15_Z1_1 | Tissue |
| 19 | 51021577 | C | T | LRRC4B | p.G465S | 0 | 140 | 0.628141 | 199 | WCM15_Z1_1 | Tissue |
| 20 | 25456818 | C | T | NINL | p.E1037K | 0 | 231 | 0.926554 | 177 | WCM15_Z1_1 | Tissue |
| 20 | 30072003 | G | A | REM1 | p.E223K | 0 | 62 | 0.111111 | 72 | WCM15_Z1_1 | Tissue |
| 20 | 33565758 | T | A | MYH7B | p.L26M | 0 | 37 | 0.12766 | 47 | WCM15_Z1_1 | Tissue |
| 2 | 17998038 | G | A | MSGN1 | p.G85S | 0 | 66 | 0.541667 | 72 | WCM15_Z1_1 | Tissue |
| 2 | 45601 | C | T | FAM110C | p.R262Q | 0 | 87 | 0.631579 | 95 | WCM15_Z1_1 | Tissue |
| 2 | 55439884 | G | C | CLHC1 | p.Q142E | 0 | 109 | 0.125 | 64 | WCM15_Z1_1 | Tissue |
| 3 | 100949982 | C | A | IMPG2 | p.V1081L | 0 | 96 | 0.183333 | 120 | WCM15_Z1_1 | Tissue |
| 5 | 150885539 | G | T | FAT2 | p.P4213T | 0 | 105 | 0.661692 | 201 | WCM15_Z1_1 | Tissue |
| 5 | 179200970 | G | T | MAML1 | p.A715S | 0 | 109 | 0.118012 | 161 | WCM15_Z1_1 | Tissue |
| 5 | 79031784 | A | C | CMYA5 | p.N2399T | 0 | 109 | 0.376344 | 93 | WCM15_Z1_1 | Tissue |
| 6 | 116837044 | C | A | FAM26E | p.S274R | 0 | 151 | 0.104294 | 163 | WCM15_Z1_1 | Tissue |
| 6 | 32781509 | C | A | HLA-DOB | p.A249S | 0 | 36 | 0.106383 | 47 | WCM15_Z1_1 | Tissue |
| 6 | 43271913 | C | A | SLC22A7 | p.T508N | 0 | 109 | 0.077465 | 142 | WCM15_Z1_1 | Tissue |
| 7 | 23213848 | T | A | KLHL7 | p.F564L | 0 | 125 | 0.463087 | 149 | WCM15_Z1_1 | Tissue |
| 8 | 131848611 | G | T | ADCY8 | p.L863M | 0 | 67 | 0.471698 | 53 | WCM15_Z1_1 | Tissue |
| 8 | 17065500 | A | G | ZDHHC2 | p.H210R | 0 | 127 | 0.957747 | 71 | WCM15_Z1_1 | Tissue |
| 8 | 95161094 | A | G | CDH17 | p.L602P | 0 | 81 | 0.104651 | 86 | WCM15_Z1_1 | Tissue |
| 9 | 17366668 | A | T | CNTLN | p.K647M | 0 | 114 | 0.051948 | 77 | WCM15_Z1_1 | Tissue |
| 9 | 75406899 | G | A | TMC1 | p.W441* | 0 | 216 | 0.880597 | 67 | WCM15_Z1_1 | Tissue |
| X | 12725014 | G | C | FRMPD4 | p.V423L | 0 | 32 | 0.104348 | 115 | WCM15_Z1_1 | Tissue |
| 11 | 103908356 | G | C | DDI1 | p.S269T | 0 | 47 | 0.209677 | 62 | WCM160 | Plasma |
| 11 | 10820640 | C | A | EIF4G2 | p.V853L | 0 | 31 | 0.068182 | 44 | WCM160 | Plasma |
| 12 | 123187305 | A | T | HCAR2 | p.L176M | 0 | 36 | 0.088889 | 45 | WCM160 | Plasma |
| 1 | 23337579 | G | A | C1orf234 | p.R61W | 0 | 37 | 0.217391 | 69 | WCM160 | Plasma |
| 12 | 9241818 | G | T | A2M | p.F916L | 0 | 64 | 0.058824 | 119 | WCM160 | Plasma |
| 14 | 20344468 | G | T | OR4K2 | p.L14F | 0 | 81 | 0.056604 | 106 | WCM160 | Plasma |
| 14 | 20871890 | G | A | TEP1 | p.R396C | 0 | 51 | 0.0625 | 64 | WCM160 | Plasma |
| 16 | 10524584 | A | G | ATF7IP2 | p.E36G | 0 | 63 | 0.290323 | 93 | WCM160 | Plasma |
| 16 | 22132374 | C | A | VWA3A | p.P401Q | 0 | 35 | 0.064516 | 93 | WCM160 | Plasma |
| 19 | 51132652 | G | A | SYT3 | p.R394W | 0 | 33 | 0.12963 | 54 | WCM160 | Plasma |
| 2 | 145157153 | T | C | ZEB2 | p.N534S | 0 | 42 | 0.098592 | 71 | WCM160 | Plasma |
| 2 | 61233705 | A | C | PUS10 | p.L152R | 0 | 55 | 0.135417 | 96 | WCM160 | Plasma |
| 4 | 126373323 | G | A | FAT4 | p.G3718S | 0 | 76 | 0.064516 | 93 | WCM160 | Plasma |
| 4 | 46973232 | C | G | GABRA4 | p.V248L | 0 | 62 | 0.075269 | 93 | WCM160 | Plasma |
| 5 | 140626723 | C | T | PCDHB15 | p.A526V | 0 | 62 | 0.056338 | 71 | WCM160 | Plasma |
| 7 | 47332452 | G | C | TNS3 | p.P1275R | 0 | 41 | 0.076923 | 65 | WCM160 | Plasma |
| 7 | 63726554 | G | T | ZNF679 | p.K181N | 0 | 75 | 0.072917 | 96 | WCM160 | Plasma |
| 7 | 77797283 | G | T | MAGI2 | p.A849D | 0 | 58 | 0.067416 | 89 | WCM160 | Plasma |
| 9 | 135801105 | C | T | TSC1 | p.E78K | 0 | 33 | 0.103896 | 77 | WCM160 | Plasma |
| X | 142718057 | G | A | SLITRK4 | p.H290Y | 0 | 45 | 0.117647 | 68 | WCM160 | Plasma |
| X | 32867862 | G | C | DMD | p.L57V | 0 | 62 | 0.150538 | 93 | WCM160 | Plasma |
| 4 | 37448316 | G | T | KIAA1239 | p.R1569L | 0 | 73 | 0.067797 | 59 | WCM160_X1_1 | Tissue |
| 6 | 56857309 | G | A | BEND6 | p.R85Q | 0 | 65 | 0.065934 | 91 | WCM160_X1_1 | Tissue |
| 11 | 103908356 | G | C | DDI1 | p.S269T | 0 | 61 | 0.209677 | 62 | WCM160_Z7_1 | Tissue |
| 11 | 55944544 | G | T | OR5J2 | p.G151C | 0 | 309 | 0.109467 | 338 | WCM160_Z7_1 | Tissue |
| 1 | 179077074 | C | G | ABL2 | p.V1110L | 0 | 268 | 0.098684 | 304 | WCM160_Z7_1 | Tissue |
| 1 | 201751699 | G | A | NAV1 | p.G687R | 0 | 92 | 0.269231 | 156 | WCM160_Z7_1 | Tissue |
| 12 | 122966631 | G | A | ZCCHC8 | p.R246W | 0 | 116 | 0.067416 | 89 | WCM160_Z7_1 | Tissue |
| 12 | 123187305 | A | T | HCAR2 | p.L176M | 0 | 159 | 0.107843 | 102 | WCM160_Z7_1 | Tissue |
| 12 | 50642451 | C | A | LIMA1 | p.K28N | 0 | 94 | 0.109589 | 73 | WCM160_Z7_1 | Tissue |
| 12 | 97137219 | T | C | C12orf55 | p.V2420A | 0 | 173 | 0.0875 | 240 | WCM160_Z7_1 | Tissue |
| 13 | 79942892 | G | T | RBM26 | p.P290T | 0 | 82 | 0.082192 | 73 | WCM160_Z7_1 | Tissue |
| 16 | 10524584 | A | G | ATF7IP2 | p.E36G | 0 | 32 | 0.392157 | 51 | WCM160_Z7_1 | Tissue |
| 16 | 89346220 | C | T | ANKRD11 | p.V2244I | 0 | 71 | 0.0625 | 96 | WCM160_Z7_1 | Tissue |
| 19 | 36515422 | G | A | CLIP3 | p.A265V | 0 | 92 | 0.096491 | 114 | WCM160_Z7_1 | Tissue |
| 19 | 51132652 | G | A | SYT3 | p.R394W | 0 | 100 | 0.154639 | 97 | WCM160_Z7_1 | Tissue |
| 19 | 56702288 | C | A | ZSCAN5B | p.Q219H | 0 | 58 | 0.12 | 50 | WCM160_Z7_1 | Tissue |
| 19 | 9061843 | C | A | MUC16 | p.E8535* | 0 | 92 | 0.206186 | 97 | WCM160_Z7_1 | Tissue |
| 2 | 239072961 | C | T | FAM132B | p.R283C | 0 | 47 | 0.168421 | 95 | WCM160_Z7_1 | Tissue |
| 2 | 61233705 | A | C | PUS10 | p.L152R | 0 | 60 | 0.196078 | 51 | WCM160_Z7_1 | Tissue |
| 3 | 179399759 | G | A | USP13 | p.V88I | 0 | 114 | 0.085938 | 128 | WCM160_Z7_1 | Tissue |
| 3 | 64666930 | C | T | ADAMTS9 | p.R209H | 0 | 163 | 0.090395 | 177 | WCM160_Z7_1 | Tissue |
| 4 | 185697802 | C | A | ACSL1 | p.V198F | 0 | 63 | 0.405797 | 69 | WCM160_Z7_1 | Tissue |
| 4 | 46973232 | C | G | GABRA4 | p.V248L | 0 | 92 | 0.129771 | 131 | WCM160_Z7_1 | Tissue |
| 7 | 47332452 | G | C | TNS3 | p.P1275R | 0 | 128 | 0.056497 | 177 | WCM160_Z7_1 | Tissue |
| 7 | 88965559 | C | G | ZNF804B | p.S1088* | 0 | 46 | 0.150685 | 73 | WCM160_Z7_1 | Tissue |
| 8 | 42024806 | G | C | AP3M2 | p.V310L | 0 | 49 | 0.082949 | 217 | WCM160_Z7_1 | Tissue |
| X | 142121979 | A | G | SPANXN4 | p.K83E | 0 | 55 | 0.135593 | 59 | WCM160_Z7_1 | Tissue |
| X | 142718057 | G | A | SLITRK4 | p.H290Y | 0 | 48 | 0.183673 | 49 | WCM160_Z7_1 | Tissue |
| 10 | 55582535 | C | T | PCDH15 | p.E1651K | 0 | 71 | 0.09 | 100 | WCM161 | Plasma |
| 12 | 121435460 | G | T | HNF1A | p.S498I | 0 | 36 | 0.066667 | 60 | WCM161 | Plasma |
| 12 | 85279766 | C | A | SLC6A15 | p.R124I | 0 | 50 | 0.058824 | 85 | WCM161 | Plasma |
| 1 | 32502610 | C | T | KHDRBS1 | p.R291W | 0 | 54 | 0.121951 | 82 | WCM161 | Plasma |
| 13 | 46701794 | C | T | LCP1 | p.E606K | 0 | 62 | 0.561224 | 98 | WCM161 | Plasma |
| 13 | 46701796 | A | C | LCP1 | p.V605G | 0 | 62 | 0.565657 | 99 | WCM161 | Plasma |
| 1 | 38288331 | G | C | MTF1 | p.S410C | 0 | 33 | 0.063492 | 63 | WCM161 | Plasma |
| 14 | 31581741 | C | A | HECTD1 | p.L2053F | 0 | 37 | 0.097222 | 72 | WCM161 | Plasma |
| 15 | 55912280 | C | T | PRTG | p.R1128Q | 0 | 72 | 0.130435 | 115 | WCM161 | Plasma |
| 15 | 63419792 | G | A | LACTB | p.D286N | 0 | 46 | 0.069444 | 72 | WCM161 | Plasma |
| 15 | 90169242 | C | T | TICRR | p.P1851L | 0 | 45 | 0.058252 | 103 | WCM161 | Plasma |
| 17 | 10316027 | T | C | MYH8 | p.Y389C | 0 | 53 | 0.118421 | 76 | WCM161 | Plasma |
| 17 | 58040371 | C | T | RNFT1 | p.V111I | 0 | 34 | 0.109375 | 64 | WCM161 | Plasma |
| 17 | 7578265 | A | G | TP53 | p.I195T | 0 | 59 | 0.405063 | 79 | WCM161 | Plasma |
| 17 | 79914822 | C | T | NOTUM | p.R275H | 0 | 53 | 0.075758 | 66 | WCM161 | Plasma |
| 18 | 25589803 | G | A | CDH2 | p.R194W | 0 | 46 | 0.090909 | 88 | WCM161 | Plasma |
| 18 | 5395676 | C | T | EPB41L3 | p.V1002M | 0 | 59 | 0.462963 | 108 | WCM161 | Plasma |
| 19 | 13054391 | A | G | CALR | p.N334S | 0 | 46 | 0.2 | 50 | WCM161 | Plasma |
| 19 | 54301601 | G | T | NLRP12 | p.H941Q | 0 | 52 | 0.169231 | 65 | WCM161 | Plasma |
| 21 | 46330230 | G | A | ITGB2 | p.S39L | 0 | 64 | 0.054054 | 74 | WCM161 | Plasma |
| 3 | 128204654 | C | T | GATA2 | p.G263R | 0 | 33 | 0.288462 | 52 | WCM161 | Plasma |
| 4 | 100572577 | G | A | RP11- | p.R1077C | 0 | 52 | 0.059406 | 101 | WCM161 | Plasma |
| | | | | 766F14.2 | | | | | | | |
| 5 | 37370969 | G | T | NUP155 | p.D37E | 0 | 47 | 0.078947 | 38 | WCM161 | Plasma |
| 6 | 56472225 | G | A | DST | p.P2190S | 0 | 52 | 0.2 | 70 | WCM161 | Plasma |
| 6 | 88853727 | C | A | CNR1 | p.D423Y | 0 | 34 | 0.147059 | 68 | WCM161 | Plasma |
| 7 | 151810392 | G | A | GALNT11 | p.R381Q | 0 | 73 | 0.111111 | 108 | WCM161 | Plasma |
| X | 151123553 | G | A | GABRE | p.R381C | 0 | 60 | 0.135593 | 118 | WCM161 | Plasma |
| 10 | 55582535 | C | T | PCDH15 | p.E1651K | 0 | 42 | 0.405405 | 37 | WCM161_Z1_1 | Tissue |
| 1 | 156811514 | C | T | INSRR | p.R1157H | 0 | 69 | 0.484211 | 95 | WCM161_Z1_1 | Tissue |
| 11 | 6422647 | G | A | APBB1 | p.R506W | 0 | 153 | 0.087432 | 183 | WCM161_Z1_1 | Tissue |
| 11 | 76371084 | C | T | LRRC32 | p.R518Q | 0 | 39 | 0.151515 | 66 | WCM161_Z1_1 | Tissue |
| 12 | 120572361 | C | T | GCN1L1 | p.R2393H | 0 | 101 | 0.271605 | 81 | WCM161_Z1_1 | Tissue |
| 13 | 46701794 | C | T | LCP1 | p.E606K | 0.007752 | 258 | 0.821149 | 766 | WCM161_Z1_1 | Tissue |
| 13 | 46701796 | A | C | LCP1 | p.V605G | 0.007843 | 255 | 0.821382 | 767 | WCM161_Z1_1 | Tissue |
| 13 | 67801938 | C | A | PCDH9 | p.R212I | 0 | 107 | 0.103448 | 58 | WCM161_Z1_1 | Tissue |
| 1 | 38288331 | G | C | MTF1 | p.S410C | 0 | 99 | 0.1625 | 80 | WCM161_Z1_1 | Tissue |
| 14 | 24686398 | T | A | NEDD8 | p.I61F | 0 | 104 | 0.198758 | 161 | WCM161_Z1_1 | Tissue |
| 14 | 45414997 | T | G | KLHL28 | p.K45N | 0 | 108 | 0.111111 | 108 | WCM161_Z1_1 | Tissue |
| 14 | 92560158 | C | G | ATXN3 | p.L84F | 0 | 69 | 0.087912 | 91 | WCM161_Z1_1 | Tissue |
| 15 | 74489709 | T | C | STRA6 | p.D79G | 0 | 106 | 0.524272 | 103 | WCM161_Z1_1 | Tissue |
| 15 | 90169242 | C | T | TICRR | p.P1851L | 0 | 95 | 0.390244 | 41 | WCM161_Z1_1 | Tissue |
| 16 | 3706746 | G | T | DNASE1 | p.R143L | 0 | 95 | 0.192053 | 151 | WCM161_Z1_1 | Tissue |
| 16 | 57503661 | G | A | POLR2C | p.V143M | 0 | 64 | 0.184466 | 103 | WCM161_Z1_1 | Tissue |
| 17 | 7578265 | A | G | TP53 | p.I195T | 0 | 199 | 0.782313 | 147 | WCM161_Z1_1 | Tissue |
| 17 | 79914822 | C | T | NOTUM | p.R275H | 0 | 150 | 0.339394 | 165 | WCM161_Z1_1 | Tissue |
| 18 | 5395676 | C | T | EPB41L3 | p.V1002M | 0 | 198 | 0.450777 | 193 | WCM161_Z1_1 | Tissue |
| 19 | 50804981 | C | T | MYH14 | p.R1804W | 0 | 36 | 0.28125 | 32 | WCM161_Z1_1 | Tissue |
| 19 | 51215331 | C | T | SHANK1 | p.R278H | 0 | 93 | 0.26 | 50 | WCM161_Z1_1 | Tissue |
| 19 | 54301601 | G | T | NLRP12 | p.H941Q | 0 | 143 | 0.417722 | 79 | WCM161_Z1_1 | Tissue |
| 2 | 186669449 | T | A | FSIP2 | p.L5139Q | 0 | 65 | 0.644444 | 45 | WCM161_Z1_1 | Tissue |
| 2 | 54858230 | C | T | SPTBN1 | p.Q1016* | 0 | 106 | 0.152542 | 118 | WCM161_Z1_1 | Tissue |
| 3 | 128204654 | C | T | GATA2 | p.G263R | 0 | 63 | 0.471698 | 53 | WCM161_Z1_1 | Tissue |
| 3 | 49698303 | C | T | BSN | p.R3009C | 0 | 135 | 0.053476 | 187 | WCM161_Z1_1 | Tissue |
| 3 | 57827029 | C | T | SLMAP | p.T117I | 0 | 56 | 0.153846 | 78 | WCM161_Z1_1 | Tissue |
| 3 | 77147468 | G | A | ROBO2 | p.R122Q | 0 | 219 | 0.144876 | 283 | WCM161_Z1_1 | Tissue |
| 3 | 97590787 | C | A | CRYBG3 | p.T250N | 0 | 216 | 0.061584 | 341 | WCM161_Z1_1 | Tissue |
| 6 | 39271778 | C | T | KCNK17 | p.A215T | 0 | 70 | 0.25 | 80 | WCM161_Z1_1 | Tissue |
| 6 | 88853727 | C | A | CNR1 | p.D423Y | 0 | 53 | 0.263889 | 72 | WCM161_Z1_1 | Tissue |
| 7 | 4285392 | G | C | SDK1 | p.E2112D | 0 | 81 | 0.317829 | 129 | WCM161_Z1_1 | Tissue |
| 7 | 87046777 | C | T | ABCB4 | p.G845S | 0 | 155 | 0.11828 | 186 | WCM161_Z1_1 | Tissue |
| 9 | 140267385 | C | T | EXD3 | p.R145H | 0 | 154 | 0.161137 | 211 | WCM161_Z1_1 | Tissue |
| 9 | 35043107 | G | A | C9orf131 | p.V161M | 0 | 102 | 0.401235 | 162 | WCM161_Z1_1 | Tissue |
| 11 | 62295576 | G | A | AHNAK | p.L2105F | 0 | 38 | 0.083333 | 36 | WCM161_Z15_1 | Tissue |
| 1 | 32502610 | C | T | KHDRBS1 | p.R291W | 0 | 129 | 0.292929 | 99 | WCM161_Z15_1 | Tissue |
| 13 | 38211227 | G | A | TRPC4 | p.T916M | 0 | 45 | 0.137931 | 116 | WCM161_Z15_1 | Tissue |
| 13 | 46701796 | A | C | LCP1 | p.V605G | 0.007843 | 255 | 0.465347 | 202 | WCM161_Z15_1 | Tissue |
| 15 | 63419792 | G | A | LACTB | p.D286N | 0 | 99 | 0.330435 | 115 | WCM161_Z15_1 | Tissue |
| 1 | 77528726 | G | A | ST6GALNAC5 | p.M282I | 0 | 53 | 0.329114 | 79 | WCM161_Z15_1 | Tissue |
| 17 | 7578265 | A | G | TP53 | p.I195T | 0 | 199 | 0.991525 | 118 | WCM161_Z15_1 | Tissue |
| 18 | 25589803 | G | A | CDH2 | p.R194W | 0 | 246 | 0.276923 | 195 | WCM161_Z15_1 | Tissue |
| 19 | 54301601 | G | T | NLRP12 | p.H941Q | 0 | 143 | 0.596154 | 52 | WCM161_Z15_1 | Tissue |
| 20 | 57428418 | C | A | GNAS | p.A33D | 0 | 67 | 0.204545 | 44 | WCM161_Z15_1 | Tissue |
| 2 | 186669449 | T | A | FSIP2 | p.L5139Q | 0 | 65 | 0.262295 | 61 | WCM161_Z15_1 | Tissue |
| 2 | 32474933 | C | T | NLRC4 | p.R667Q | 0 | 64 | 0.258824 | 85 | WCM161_Z15_1 | Tissue |
| 2 | 54858230 | C | T | SPTBN1 | p.Q1016* | 0 | 106 | 0.829787 | 47 | WCM161_Z15_1 | Tissue |
| 3 | 128204654 | C | T | GATA2 | p.G263R | 0 | 63 | 0.540541 | 37 | WCM161_Z15_1 | Tissue |
| 4 | 100572577 | G | A | RP11- | p.R1077C | 0 | 63 | 0.21831 | 142 | WCM161_Z15_1 | Tissue |
| | | | | 766F14.2 | | | | | | | |
| 6 | 88853727 | C | A | CNR1 | p.D423Y | 0 | 53 | 0.375 | 48 | WCM161_Z15_1 | Tissue |
| 8 | 118849393 | G | A | EXT1 | p.P337L | 0 | 155 | 0.119048 | 168 | WCM161_Z15_1 | Tissue |
| 9 | 35043107 | G | A | C9orf131 | p.V161M | 0 | 102 | 0.497041 | 169 | WCM161_Z15_1 | Tissue |
| 10 | 55582535 | C | T | PCDH15 | p.E1651K | 0 | 42 | 0.26 | 50 | WCM161_Z6_1 | Tissue |
| 11 | 76371084 | C | T | LRRC32 | p.R518Q | 0 | 39 | 0.136364 | 66 | WCM161_Z6_1 | Tissue |
| 1 | 201179977 | G | A | IGFN1 | p.G1986S | 0 | 34 | 0.125 | 56 | WCM161_Z6_1 | Tissue |
| 12 | 120572361 | C | T | GCN1L1 | p.R2393H | 0 | 101 | 0.15625 | 96 | WCM161_Z6_1 | Tissue |
| 13 | 46701794 | C | T | LCP1 | p.E606K | 0.007752 | 258 | 0.769366 | 568 | WCM161_Z6_1 | Tissue |
| 13 | 46701796 | A | C | LCP1 | p.V605G | 0.007843 | 255 | 0.768014 | 569 | WCM161_Z6_1 | Tissue |
| 1 | 38288331 | G | C | MTF1 | p.S410C | 0 | 99 | 0.072727 | 110 | WCM161_Z6_1 | Tissue |
| 14 | 24686398 | T | A | NEDD8 | p.I61F | 0 | 104 | 0.067308 | 104 | WCM161_Z6_1 | Tissue |
| 14 | 24801858 | G | A | ADCY4 | p.A130V | 0 | 39 | 0.102564 | 39 | WCM161_Z6_1 | Tissue |
| 15 | 74489709 | T | C | STRA6 | p.D79G | 0 | 106 | 0.297297 | 111 | WCM161_Z6_1 | Tissue |
| 15 | 90169242 | C | T | TICRR | p.P1851L | 0 | 95 | 0.141026 | 78 | WCM161_Z6_1 | Tissue |
| 16 | 57503661 | G | A | POLR2C | p.V143M | 0 | 64 | 0.108434 | 83 | WCM161_Z6_1 | Tissue |
| 17 | 7578265 | A | G | TP53 | p.I195T | 0 | 199 | 0.557692 | 208 | WCM161_Z6_1 | Tissue |
| 17 | 79914822 | C | T | NOTUM | p.R275H | 0 | 150 | 0.07971 | 138 | WCM161_Z6_1 | Tissue |
| 2 | 186669449 | T | A | FSIP2 | p.L5139Q | 0 | 65 | 0.322034 | 59 | WCM161_Z6_1 | Tissue |
| 2 | 54858230 | C | T | SPTBN1 | p.Q1016* | 0 | 106 | 0.142857 | 91 | WCM161_Z6_1 | Tissue |
| 3 | 123385148 | C | T | MYLK | p.R1250H | 0 | 38 | 0.078431 | 51 | WCM161_Z6_1 | Tissue |
| 3 | 128204654 | C | T | GATA2 | p.G263R | 0 | 63 | 0.401709 | 117 | WCM161_Z6_1 | Tissue |
| 3 | 51431267 | G | T | RBM15B | p.E813* | 0 | 62 | 0.337349 | 83 | WCM161_Z6_1 | Tissue |
| 5 | 23509657 | C | T | PRDM9 | p.R50C | 0 | 34 | 0.075 | 40 | WCM161_Z6_1 | Tissue |
| 6 | 39271778 | C | T | KCNK17 | p.A215T | 0 | 70 | 0.240506 | 79 | WCM161_Z6_1 | Tissue |
| 7 | 151810392 | G | A | GALNT11 | p.R381Q | 0 | 81 | 0.084746 | 59 | WCM161_Z6_1 | Tissue |
| 7 | 4285392 | G | C | SDK1 | p.E2112D | 0 | 81 | 0.07362 | 163 | WCM161_Z6_1 | Tissue |
| 8 | 77766195 | A | C | ZFHX4 | p.E2346D | 0 | 46 | 0.142857 | 98 | WCM161_Z6_1 | Tissue |
| 9 | 140267385 | C | T | EXD3 | p.R145H | 0 | 154 | 0.148936 | 235 | WCM161_Z6_1 | Tissue |
| 9 | 35043107 | G | A | C9orf131 | p.V161M | 0 | 102 | 0.310345 | 145 | WCM161_Z6_1 | Tissue |
| X | 79281236 | G | A | TBX22 | p.R198H | 0 | 57 | 0.090909 | 66 | WCM161_Z6_1 | Tissue |
| 1 | 16890515 | C | A | NBPF1 | p.V1115L | 0 | 104 | 0.055901 | 161 | WCM162 | Plasma |
| 1 | 169679606 | T | C | SELL | p.K32R | 0 | 38 | 0.637255 | 102 | WCM162 | Plasma |
| 12 | 13232864 | A | G | KIAA1467 | p.Q595R | 0 | 40 | 0.223214 | 112 | WCM162 | Plasma |
| 12 | 57871258 | C | T | ARHGAP9 | p.R247H | 0 | 31 | 0.255319 | 94 | WCM162 | Plasma |
| 14 | 53112919 | G | C | ERO1L | p.F433L | 0 | 51 | 0.063636 | 110 | WCM162 | Plasma |
| 14 | 73464896 | C | A | ZFYVE1 | p.G204V | 0 | 39 | 0.265487 | 113 | WCM162 | Plasma |
| 16 | 51175796 | G | A | SALL1 | p.L113F | 0 | 52 | 0.323529 | 136 | WCM162 | Plasma |
| 17 | 42463069 | G | T | ITGA2B | p.Q142K | 0 | 40 | 0.505376 | 93 | WCM162 | Plasma |
| 18 | 12122424 | G | A | ANKRD62 | p.V455I | 0 | 40 | 0.239437 | 71 | WCM162 | Plasma |
| 2 | 177016375 | C | T | HOXD4 | p.S5L | 0 | 46 | 0.051471 | 136 | WCM162 | Plasma |
| 2 | 234106805 | C | A | INPP5D | p.P908T | 0 | 33 | 0.068627 | 102 | WCM162 | Plasma |
| 2 | 88828747 | C | T | TEX37 | p.R100C | 0 | 40 | 0.054054 | 111 | WCM162 | Plasma |
| 3 | 134338044 | C | T | KY | p.R219Q | 0 | 33 | 0.33 | 100 | WCM162 | Plasma |
| 4 | 20535275 | C | T | SLIT2 | p.T590M | 0 | 49 | 0.33913 | 115 | WCM162 | Plasma |
| 4 | 36134981 | C | G | ARAP2 | p.L1098F | 0 | 38 | 0.173469 | 98 | WCM162 | Plasma |
| 7 | 4304776 | G | T | SDK1 | p.E2134D | 0 | 53 | 0.461078 | 167 | WCM162 | Plasma |
| 8 | 143994104 | C | A | CYP11B2 | p.A414S | 0 | 43 | 0.088496 | 113 | WCM162 | Plasma |
| 9 | 35649960 | G | C | SIT1 | p.S159C | 0 | 40 | 0.151316 | 152 | WCM162 | Plasma |
| 11 | 118375359 | A | G | KMT2A | p.I2915V | 0 | 83 | 0.362069 | 58 | WCM162_Z1_1 | Tissue |
| 1 | 169679606 | T | C | SELL | p.K32R | 0 | 80 | 0.883333 | 60 | WCM162_Z1_1 | Tissue |
| 1 | 182443421 | A | G | RGSL1 | p.N392S | 0 | 210 | 0.176471 | 170 | WCM162_Z1_1 | Tissue |
| 11 | 8643323 | G | A | TRIM66 | p.R866* | 0 | 68 | 0.385965 | 57 | WCM162_Z1_1 | Tissue |
| 1 | 201042709 | G | C | CACNA1S | p.P709A | 0 | 76 | 0.260274 | 73 | WCM162_Z1_1 | Tissue |
| 12 | 10342624 | G | A | TMEM52B | p.R146H | 0 | 159 | 0.238095 | 147 | WCM162_Z1_1 | Tissue |
| 12 | 13232864 | A | G | KIAA1467 | p.Q595R | 0 | 91 | 0.591837 | 49 | WCM162_Z1_1 | Tissue |
| 14 | 94962975 | A | G | SERPINA12 | p.W214R | 0 | 81 | 0.5 | 78 | WCM162_Z1_1 | Tissue |
| 14 | 99977099 | C | T | CCNK | p.R575W | 0 | 61 | 0.104651 | 86 | WCM162_Z1_1 | Tissue |
| 15 | 31664300 | C | T | KLF13 | p.T222M | 0 | 66 | 0.069767 | 43 | WCM162_Z1_1 | Tissue |
| 16 | 2814185 | C | G | SRRM2 | p.S1219C | 0 | 117 | 0.185484 | 124 | WCM162_Z1_1 | Tissue |
| 1 | 6293582 | C | A | ICMT | p.A136S | 0 | 57 | 0.065217 | 46 | WCM162_Z1_1 | Tissue |
| 17 | 42463069 | G | T | ITGA2B | p.Q142K | 0 | 105 | 0.8 | 80 | WCM162_Z1_1 | Tissue |
| 17 | 46608005 | C | A | HOXB1 | p.A88S | 0.009524 | 105 | 0.132353 | 136 | WCM162_Z1_1 | Tissue |
| 17 | 79171676 | T | C | AZI1 | p.S498G | 0 | 104 | 0.354286 | 175 | WCM162_Z1_1 | Tissue |
| 1 | 91405745 | T | C | ZNF644 | p.K389R | 0 | 162 | 0.112782 | 266 | WCM162_Z1_1 | Tissue |
| 19 | 49686118 | C | T | TRPM4 | p.A516V | 0 | 42 | 0.288462 | 52 | WCM162_Z1_1 | Tissue |
| 19 | 52909857 | G | T | ZNF528 | p.D78Y | 0 | 97 | 0.113924 | 79 | WCM162_Z1_1 | Tissue |
| 19 | 5456584 | G | T | ZNRF4 | p.S361I | 0 | 61 | 0.123077 | 65 | WCM162_Z1_1 | Tissue |
| 2 | 179482934 | C | T | TTN | p.E14110K | 0 | 75 | 0.294872 | 78 | WCM162_Z1_1 | Tissue |
| 22 | 38017651 | A | G | GGA1 | p.H186R | 0 | 170 | 0.229358 | 109 | WCM162_Z1_1 | Tissue |
| 2 | 37473249 | G | C | NDUFAF7 | p.E283Q | 0 | 56 | 0.209302 | 43 | WCM162_Z1_1 | Tissue |
| 3 | 134338044 | C | T | KY | p.R219Q | 0 | 184 | 0.156863 | 102 | WCM162_Z1_1 | Tissue |
| 3 | 45972765 | A | C | FYCO1 | p.V1350G | 0 | 43 | 0.368421 | 38 | WCM162_Z1_1 | Tissue |
| 4 | 1360144 | G | A | UVSSA | p.D405N | 0 | 52 | 0.179487 | 39 | WCM162_Z1_1 | Tissue |
| 4 | 185681621 | G | C | ACSL1 | p.H558D | 0 | 63 | 0.282051 | 39 | WCM162_Z1_1 | Tissue |
| 4 | 20535275 | C | T | SLIT2 | p.T590M | 0 | 69 | 0.27027 | 74 | WCM162_Z1_1 | Tissue |
| 4 | 36134981 | C | G | ARAP2 | p.L1098F | 0 | 131 | 0.138614 | 101 | WCM162_Z1_1 | Tissue |
| 4 | 4204201 | G | A | OTOP1 | p.T235I | 0 | 148 | 0.186275 | 102 | WCM162_Z1_1 | Tissue |
| 4 | 42467008 | T | C | ATP8A1 | p.N804D | 0 | 159 | 0.34 | 100 | WCM162_Z1_1 | Tissue |
| 5 | 140773380 | C | T | PCDHGA8 | p.L334F | 0 | 88 | 0.395833 | 48 | WCM162_Z1_1 | Tissue |
| 5 | 149500792 | C | T | PDGFRB | p.G813D | 0 | 37 | 0.205882 | 34 | WCM162_Z1_1 | Tissue |
| 5 | 180472533 | C | G | BTNL9 | p.S15W | 0 | 137 | 0.5 | 74 | WCM162_Z1_1 | Tissue |
| 5 | 482835 | C | T | SLC9A3 | p.R395H | 0 | 318 | 0.339768 | 259 | WCM162_Z1_1 | Tissue |
| 5 | 63894231 | G | C | RGS7BP | p.R217S | 0 | 84 | 0.087719 | 57 | WCM162_Z1_1 | Tissue |
| 6 | 100055043 | C | A | PRDM13 | p.P45T | 0 | 139 | 0.148148 | 297 | WCM162_Z1_1 | Tissue |
| 6 | 7230926 | C | G | RREB1 | p.P865R | 0 | 104 | 0.116883 | 77 | WCM162_Z1_1 | Tissue |
| 7 | 121651490 | C | T | PTPRZ1 | p.T797M | 0 | 150 | 0.09901 | 202 | WCM162_Z1_1 | Tissue |
| 7 | 148802170 | G | T | ZNF425 | p.Q265K | 0 | 55 | 0.305882 | 85 | WCM162_Z1_1 | Tissue |
| 7 | 31120231 | G | T | ADCYAP1R1 | p.W90L | 0 | 76 | 0.186275 | 102 | WCM162_Z1_1 | Tissue |
| 7 | 4304776 | G | T | SDK1 | p.E2134D | 0 | 197 | 0.561856 | 194 | WCM162_Z1_1 | Tissue |
| 8 | 133141609 | G | T | KCNQ3 | p.T840K | 0 | 35 | 0.058824 | 51 | WCM162_Z1_1 | Tissue |
| 8 | 77617806 | A | G | ZFHX4 | p.I495V | 0 | 93 | 0.45283 | 53 | WCM162_Z1_1 | Tissue |
| 9 | 100086128 | C | G | CCDC180 | p.F621L | 0 | 185 | 0.366667 | 150 | WCM162_Z1_1 | Tissue |
| 9 | 113308556 | C | G | SVEP1 | p.S268T | 0 | 97 | 0.054054 | 74 | WCM162_Z1_1 | Tissue |
| 9 | 35649960 | G | C | SIT1 | p.S159C | 0 | 76 | 0.20438 | 137 | WCM162_Z1_1 | Tissue |
| 11 | 8643323 | G | A | TRIM66 | p.R866* | 0 | 68 | 0.059524 | 84 | WCM162_Z9_1 | Tissue |
| 13 | 25882008 | G | T | NUPL1 | p.A58S | 0 | 113 | 0.074766 | 107 | WCM162_Z9_1 | Tissue |
| 13 | 39264439 | A | T | FREM2 | p.E986D | 0 | 35 | 0.096774 | 31 | WCM162_Z9_1 | Tissue |
| 14 | 94962975 | A | G | SERPINA12 | p.W214R | 0 | 81 | 0.103448 | 87 | WCM162_Z9_1 | Tissue |
| 17 | 42463069 | G | T | ITGA2B | p.Q142K | 0 | 105 | 0.178082 | 73 | WCM162_Z9_1 | Tissue |
| 17 | 46608005 | C | A | HOXB1 | p.A88S | 0.009524 | 105 | 0.056604 | 106 | WCM162_Z9_1 | Tissue |
| 18 | 28587094 | T | C | DSC3 | p.D556G | 0 | 62 | 0.06 | 50 | WCM162_Z9_1 | Tissue |
| 19 | 12297713 | G | A | ZNF136 | p.G174R | 0 | 56 | 0.140351 | 57 | WCM162_Z9_1 | Tissue |
| 19 | 57176325 | C | T | ZNF835 | p.R81Q | 0 | 125 | 0.07438 | 121 | WCM162_Z9_1 | Tissue |
| 2 | 102505261 | A | T | MAP4K4 | p.Y1167F | 0 | 218 | 0.055276 | 199 | WCM162_Z9_1 | Tissue |
| 21 | 47402557 | T | G | COL6A1 | p.V36G | 0 | 65 | 0.114286 | 70 | WCM162_Z9_1 | Tissue |
| 2 | 3691060 | C | T | COLEC11 | p.R119C | 0 | 72 | 0.057692 | 52 | WCM162_Z9_1 | Tissue |
| 4 | 36134981 | C | G | ARAP2 | p.L1098F | 0 | 131 | 0.096491 | 114 | WCM162_Z9_1 | Tissue |
| 7 | 4304776 | G | T | SDK1 | p.E2134D | 0 | 197 | 0.215596 | 218 | WCM162_Z9_1 | Tissue |
| 9 | 100086128 | C | G | CCDC180 | p.F621L | 0 | 185 | 0.050847 | 177 | WCM162_Z9_1 | Tissue |
| 13 | 48919317 | T | C | RB1 | p.L161P | 0 | 60 | 0.342105 | 38 | WCM163 | Plasma |
| 16 | 83813639 | C | G | CDH13 | p.P583R | 0 | 70 | 0.327869 | 61 | WCM163 | Plasma |
| 17 | 41960701 | G | A | MPP2 | p.T82M | 0 | 66 | 0.145833 | 96 | WCM163 | Plasma |
| 19 | 12059500 | A | C | ZNF700 | p.T221P | 0 | 74 | 0.409524 | 105 | WCM163 | Plasma |
| 19 | 47844555 | G | A | C5AR2 | p.V167M | 0 | 50 | 0.08 | 50 | WCM163 | Plasma |
| 22 | 24562733 | G | T | CABIN1 | p.A1712S | 0 | 60 | 0.306122 | 49 | WCM163 | Plasma |
| 4 | 119252962 | C | G | PRSS12 | p.E294Q | 0 | 50 | 0.175439 | 57 | WCM163 | Plasma |
| 4 | 144826647 | A | G | GYPE | p.I5T | 0 | 58 | 0.223404 | 94 | WCM163 | Plasma |
| 5 | 40937708 | T | G | C7 | p.F161L | 0 | 74 | 0.137931 | 87 | WCM163 | Plasma |
| 7 | 79082393 | C | T | MAGI2 | p.V82M | 0 | 77 | 0.168224 | 107 | WCM163 | Plasma |
| 8 | 3141672 | A | T | CSMD1 | p.F1384I | 0 | 42 | 0.351852 | 54 | WCM163 | Plasma |
| 11 | 57068506 | G | A | TNKS1BP1 | p.R1661C | 0 | 73 | 0.142857 | 42 | WCM163_Z7_1 | Tissue |
| 11 | 63258367 | G | A | HRASLS5 | p.P47L | 0 | 171 | 0.342657 | 143 | WCM163_Z7_1 | Tissue |
| 13 | 42273224 | C | A | VWA8 | p.V1183F | 0 | 43 | 0.935484 | 31 | WCM163_Z7_1 | Tissue |
| 1 | 53675828 | G | A | CPT2 | p.R161Q | 0 | 54 | 0.942857 | 35 | WCM163_Z7_1 | Tissue |
| 15 | 42028541 | A | G | MGA | p.N1360S | 0 | 82 | 0.308642 | 81 | WCM163_Z7_1 | Tissue |
| 1 | 62237255 | G | A | INADL | p.S226N | 0 | 78 | 0.222222 | 135 | WCM163_Z7_1 | Tissue |
| 16 | 85015495 | C | T | ZDHHC7 | p.E141K | 0 | 88 | 0.088235 | 34 | WCM163_Z7_1 | Tissue |
| 18 | 76754200 | G | A | SALL3 | p.V737M | 0 | 53 | 0.254902 | 51 | WCM163_Z7_1 | Tissue |
| 19 | 12059500 | A | C | ZNF700 | p.T221P | 0 | 62 | 0.4375 | 96 | WCM163_Z7_1 | Tissue |
| 19 | 19767554 | A | C | ATP13A1 | p.L333R | 0 | 83 | 0.31068 | 103 | WCM163_Z7_1 | Tissue |
| 19 | 47844555 | G | A | C5AR2 | p.V167M | 0 | 71 | 0.425532 | 47 | WCM163_Z7_1 | Tissue |
| 2 | 160637506 | G | A | CD302 | p.A61V | 0 | 68 | 0.395833 | 48 | WCM163_Z7_1 | Tissue |
| 22 | 44579216 | C | A | PARVG | p.P3T | 0 | 93 | 0.054545 | 55 | WCM163_Z7_1 | Tissue |
| 3 | 32180154 | C | A | GPD1L | p.H101N | 0 | 55 | 0.319149 | 47 | WCM163_Z7_1 | Tissue |
| 4 | 119252962 | C | G | PRSS12 | p.E294Q | 0 | 122 | 0.090909 | 77 | WCM163_Z7_1 | Tissue |
| 4 | 13606068 | T | A | BOD1L1 | p.N819I | 0 | 78 | 0.191176 | 68 | WCM163_Z7_1 | Tissue |
| 5 | 160047538 | C | A | ATP10B | p.E744D | 0 | 145 | 0.505435 | 184 | WCM163_Z7_1 | Tissue |
| 5 | 173359456 | G | A | CPEB4 | p.R404H | 0 | 98 | 0.248227 | 141 | WCM163_Z7_1 | Tissue |
| 5 | 176304669 | C | T | UNC5A | p.R534C | 0 | 60 | 0.265823 | 79 | WCM163_Z7_1 | Tissue |
| 7 | 141424950 | C | T | WEE2 | p.P449L | 0 | 199 | 0.311881 | 202 | WCM163_Z7_1 | Tissue |
| 7 | 94176475 | G | C | CASD1 | p.L567F | 0 | 31 | 0.333333 | 39 | WCM163_Z7_1 | Tissue |
| 8 | 3141672 | A | T | CSMD1 | p.F1384I | 0 | 68 | 0.432836 | 67 | WCM163_Z7_1 | Tissue |
| X | 152845721 | G | A | ATP2B3 | p.G1210R | 0 | 32 | 0.944444 | 36 | WCM163_Z7_1 | Tissue |
| X | 26212507 | C | A | MAGEB6 | p.Q182K | 0 | 35 | 0.055556 | 54 | WCM163_Z7_1 | Tissue |
| X | 71895976 | C | A | PHKA1 | p.D188Y | 0 | 53 | 0.076923 | 78 | WCM163_Z7_1 | Tissue |
| 1 | 112031565 | T | C | ADORA3 | p.Y180C | 0 | 38 | 0.096774 | 62 | WCM164 | Plasma |
| 12 | 72092752 | A | G | TMEM19 | p.Y237C | 0 | 41 | 0.096386 | 83 | WCM164 | Plasma |
| 17 | 7577574 | T | C | TP53 | p.Y236C | 0 | 33 | 0.109375 | 64 | WCM164 | Plasma |
| 10 | 135086499 | G | A | ADAM8 | p.R200C | 0 | 64 | 0.098039 | 51 | WCM164_X1_1 | Tissue |
| 10 | 27324385 | G | T | ANKRD26 | p.S998R | 0 | 64 | 0.12381 | 105 | WCM164_X1_1 | Tissue |
| 1 | 22816663 | G | T | ZBTB40 | p.L74F | 0 | 52 | 0.090909 | 33 | WCM164_X1_1 | Tissue |
| 12 | 49176698 | C | T | ADCY6 | p.A174T | 0 | 55 | 0.180556 | 72 | WCM164_X1_1 | Tissue |
| 12 | 8083127 | T | C | SLC2A3 | p.S208G | 0 | 165 | 0.07027 | 185 | WCM164_X1_1 | Tissue |
| 13 | 114778646 | T | C | RASA3 | p.N495S | 0 | 120 | 0.463415 | 123 | WCM164_X1_1 | Tissue |
| 13 | 114778664 | G | A | RASA3 | p.P489L | 0 | 109 | 0.478261 | 115 | WCM164_X1_1 | Tissue |
| 13 | 115009458 | C | G | CDC16 | p.T254S | 0 | 108 | 0.139241 | 79 | WCM164_X1_1 | Tissue |
| 14 | 101010278 | G | A | BEGAIN | p.R90C | 0 | 103 | 0.325397 | 126 | WCM164_X1_1 | Tissue |
| 1 | 43777674 | G | A | TIE1 | p.S501N | 0 | 190 | 0.375 | 168 | WCM164_X1_1 | Tissue |
| 17 | 7577574 | T | C | TP53 | p.Y236C | 0 | 195 | 0.517442 | 172 | WCM164_X1_1 | Tissue |
| 20 | 55027649 | G | T | CASS4 | p.E473* | 0 | 198 | 0.165217 | 115 | WCM164_X1_1 | Tissue |
| 2 | 158412656 | T | C | ACVR1C | p.K165E | 0 | 32 | 0.135135 | 37 | WCM164_X1_1 | Tissue |
| 2 | 33623439 | G | A | LTBP1 | p.E1665K | 0 | 48 | 0.326531 | 49 | WCM164_X1_1 | Tissue |
| 5 | 72469209 | C | A | TMEM174 | p.L47M | 0 | 191 | 0.116279 | 172 | WCM164_X1_1 | Tissue |
| 6 | 91278290 | G | T | MAP3K7 | p.A95D | 0 | 61 | 0.153846 | 91 | WCM164_X1_1 | Tissue |
| 7 | 150839597 | C | T | AGAP3 | p.L386F | 0 | 96 | 0.150685 | 146 | WCM164_X1_1 | Tissue |
| 7 | 76029752 | C | T | SRCRB4D | p.R109Q | 0 | 50 | 0.142857 | 49 | WCM164_X1_1 | Tissue |
| 9 | 123903763 | T | G | CNTRL | p.V863G | 0 | 126 | 0.179012 | 162 | WCM164_X1_1 | Tissue |
| X | 139587119 | G | T | SOX3 | p.A36D | 0 | 39 | 0.2 | 40 | WCM164_X1_1 | Tissue |
| 15 | 43712787 | T | C | TP53BP1 | p.D1461G | 0 | 33 | 0.090909 | 55 | WCM169 | Plasma |
| 15 | 43748917 | C | G | TP53BP1 | p.S625T | 0 | 33 | 0.105263 | 57 | WCM169 | Plasma |
| 15 | 43748918 | T | C | TP53BP1 | p.S625G | 0 | 32 | 0.105263 | 57 | WCM169 | Plasma |
| 15 | 43783909 | C | G | TP53BP1 | p.S105T | 0 | 53 | 0.090909 | 77 | WCM169 | Plasma |
| 15 | 43783910 | T | C | TP53BP1 | p.S105G | 0 | 53 | 0.090909 | 77 | WCM169 | Plasma |
| 11 | 128842742 | C | A | ARHGAP32 | p.R1206L | 0 | 58 | 0.096774 | 31 | WCM169_X1_1 | Tissue |
| 11 | 55563663 | G | T | OR5D14 | p.C211F | 0 | 67 | 0.052632 | 57 | WCM169_X1_1 | Tissue |
| 11 | 66083529 | G | T | CD248 | p.Q324K | 0 | 98 | 0.078947 | 76 | WCM169_X1_1 | Tissue |
| 12 | 113303281 | G | T | RPH3A | p.C98F | 0 | 77 | 0.065217 | 92 | WCM169_X1_1 | Tissue |
| 12 | 54448685 | C | A | HOXC4 | p.T164N | 0 | 126 | 0.061224 | 49 | WCM169_X1_1 | Tissue |
| 14 | 102661325 | G | T | WDR20 | p.Q100H | 0 | 59 | 0.081081 | 37 | WCM169_X1_1 | Tissue |
| 14 | 103336598 | G | T | TRAF3 | p.K20N | 0 | 70 | 0.073171 | 82 | WCM169_X1_1 | Tissue |
| 16 | 15729680 | C | A | KIAA0430 | p.G222C | 0 | 122 | 0.067416 | 89 | WCM169_X1_1 | Tissue |
| 19 | 15302421 | C | T | NOTCH3 | p.A284T | 0 | 58 | 0.084507 | 71 | WCM169_X1_1 | Tissue |
| 19 | 17722640 | C | A | UNC13A | p.G1528V | 0 | 137 | 0.054795 | 146 | WCM169_X1_1 | Tissue |
| 19 | 6697381 | C | A | C3 | p.G924C | 0 | 72 | 0.08 | 75 | WCM169_X1_1 | Tissue |
| 3 | 12422946 | G | T | PPARG | p.A146S | 0 | 155 | 0.0625 | 64 | WCM169_X1_1 | Tissue |
| 3 | 152163110 | G | T | MBNL1 | p.E197* | 0 | 56 | 0.061224 | 49 | WCM169_X1_1 | Tissue |
| 3 | 184039299 | G | T | EIF4G1 | p.E309D | 0 | 88 | 0.096774 | 31 | WCM169_X1_1 | Tissue |
| 5 | 141331076 | G | T | PCDH12 | p.A987D | 0 | 126 | 0.070175 | 114 | WCM169_X1_1 | Tissue |
| 7 | 100817863 | C | A | NAT16 | p.G76C | 0 | 50 | 0.077922 | 77 | WCM169_X1_1 | Tissue |
| 7 | 148801389 | G | A | ZNF425 | p.P525L | 0 | 57 | 0.0625 | 48 | WCM169_X1_1 | Tissue |
| 7 | 44149644 | G | A | AEBP1 | p.R394H | 0 | 101 | 0.117647 | 51 | WCM169_X1_1 | Tissue |
| X | 12701695 | G | A | FRMPD4 | p.G188S | 0 | 101 | 0.052174 | 115 | WCM169_X1_1 | Tissue |
| 19 | 58564971 | G | A | ZSCAN1 | p.R260H | 0 | 32 | 0.092105 | 76 | WCM170 | Plasma |
| 7 | 94047838 | G | A | COL1A2 | p.G667S | 0 | 33 | 0.074074 | 81 | WCM170 | Plasma |
| 9 | 32633273 | C | T | TAF1L | p.A769T | 0 | 34 | 0.06383 | 94 | WCM170 | Plasma |
| 10 | 101823439 | C | T | CPN1 | p.G268D | 0 | 60 | 0.081081 | 37 | WCM170_Z1_1 | Tissue |
| 10 | 48413661 | C | T | GDF2 | p.V403I | 0 | 128 | 0.191489 | 94 | WCM170_Z1_1 | Tissue |
| 10 | 6258698 | G | C | PFKFB3 | p.E132D | 0 | 242 | 0.238281 | 256 | WCM170_Z1_1 | Tissue |
| 10 | 75562812 | G | A | NDST2 | p.R781C | 0 | 76 | 0.333333 | 108 | WCM170_Z1_1 | Tissue |
| 1 | 15679397 | G | A | FHAD1 | p.E805K | 0 | 199 | 0.115385 | 182 | WCM170_Z1_1 | Tissue |
| 11 | 6150467 | G | T | OR56B3P | p.D210Y | 0 | 243 | 0.137725 | 167 | WCM170_Z1_1 | Tissue |
| 1 | 172558877 | G | A | SUCO | p.G879E | 0 | 60 | 0.06 | 50 | WCM170_Z1_1 | Tissue |
| 1 | 179521762 | T | G | NPHS2 | p.E283D | 0 | 126 | 0.181034 | 116 | WCM170_Z1_1 | Tissue |
| 1 | 20416368 | C | T | PLA2G5 | p.A91V | 0 | 191 | 0.16129 | 155 | WCM170_Z1_1 | Tissue |
| 1 | 223983752 | G | A | TP53BP2 | p.P830L | 0 | 86 | 0.302083 | 96 | WCM170_Z1_1 | Tissue |
| 12 | 27916182 | G | T | MANSC4 | p.T171K | 0 | 117 | 0.090909 | 88 | WCM170_Z1_1 | Tissue |
| 12 | 56992988 | G | A | BAZ2A | p.S1778L | 0 | 112 | 0.117978 | 178 | WCM170_Z1_1 | Tissue |
| 14 | 102506595 | T | G | DYNC1H1 | p.F3905V | 0 | 179 | 0.18797 | 133 | WCM170_Z1_1 | Tissue |
| 16 | 20492232 | G | A | ACSM2A | p.V500I | 0 | 38 | 0.297297 | 37 | WCM170_Z1_1 | Tissue |
| 1 | 62593744 | C | T | INADL | p.A1715V | 0 | 167 | 0.056452 | 124 | WCM170_Z1_1 | Tissue |
| 19 | 58564971 | G | A | ZSCAN1 | p.R260H | 0 | 187 | 0.166667 | 126 | WCM170_Z1_1 | Tissue |
| 21 | 27102057 | C | T | ATP5J | p.V17I | 0 | 228 | 0.191489 | 141 | WCM170_Z1_1 | Tissue |
| 2 | 229890413 | C | T | PID1 | p.G230R | 0 | 70 | 0.244275 | 131 | WCM170_Z1_1 | Tissue |
| 3 | 127358327 | G | T | PODXL2 | p.E104* | 0 | 157 | 0.178571 | 140 | WCM170_Z1_1 | Tissue |
| 4 | 159782540 | G | T | FNIP2 | p.V399L | 0 | 87 | 0.050847 | 59 | WCM170_Z1_1 | Tissue |
| 4 | 39347026 | T | A | RFC1 | p.D68V | 0 | 98 | 0.078125 | 64 | WCM170_Z1_1 | Tissue |
| 5 | 134060701 | C | A | SEC24A | p.Q1067K | 0 | 79 | 0.083333 | 72 | WCM170_Z1_1 | Tissue |
| 7 | 150695718 | C | A | NOS3 | p.Q256K | 0 | 221 | 0.386139 | 202 | WCM170_Z1_1 | Tissue |
| 7 | 57529622 | G | T | ZNF716 | p.M485I | 0 | 126 | 0.157895 | 133 | WCM170_Z1_1 | Tissue |
| 7 | 73280028 | C | T | WBSCR28 | p.T208I | 0 | 128 | 0.142857 | 161 | WCM170_Z1_1 | Tissue |
| 9 | 127316832 | G | A | NR6A1 | p.R54W | 0 | 56 | 0.0625 | 48 | WCM170_Z1_1 | Tissue |
| 9 | 32633273 | C | T | TAF1L | p.A769T | 0 | 63 | 0.083333 | 48 | WCM170_Z1_1 | Tissue |
| 10 | 48413661 | C | T | GDF2 | p.V403I | 0 | 128 | 0.32287 | 223 | WCM170_Z16_1 | Tissue |
| 10 | 75562812 | G | A | NDST2 | p.R781C | 0 | 76 | 0.197674 | 172 | WCM170_Z16_1 | Tissue |
| 11 | 112101358 | G | T | PTS | p.A66S | 0 | 61 | 0.068182 | 44 | WCM170_Z16_1 | Tissue |
| 1 | 15679397 | G | A | FHAD1 | P.E805K | 0 | 199 | 0.366492 | 191 | WCM170_Z16_1 | Tissue |
| 11 | 6150467 | G | T | OR56B3P | p.D210Y | 0 | 243 | 0.412556 | 223 | WCM170_Z16_1 | Tissue |
| 12 | 111884588 | C | G | SH2B3 | p.S255C | 0 | 50 | 0.389474 | 95 | WCM170_Z16_1 | Tissue |
| 12 | 132549292 | C | T | EP400 | p.P2841L | 0 | 104 | 0.230263 | 152 | WCM170_Z16_1 | Tissue |
| 1 | 223983752 | G | A | TP53BP2 | p.P830L | 0 | 86 | 0.357143 | 98 | WCM170_Z16_1 | Tissue |
| 12 | 50232759 | G | C | BCDIN3D | p.P92A | 0 | 48 | 0.274194 | 62 | WCM170_Z16_1 | Tissue |
| 12 | 56992988 | G | A | BAZ2A | p.S1778L | 0 | 112 | 0.305164 | 213 | WCM170_Z16_1 | Tissue |
| 15 | 43476731 | T | A | TMEM62 | p.S627T | 0 | 184 | 0.131579 | 114 | WCM170_Z16_1 | Tissue |
| 16 | 20492232 | G | A | ACSM2A | p.V500I | 0 | 38 | 0.402985 | 67 | WCM170_Z16_1 | Tissue |
| 16 | 67680412 | G | T | RLTPR | p.S153I | 0 | 73 | 0.086957 | 69 | WCM170_Z16_1 | Tissue |
| 18 | 21708929 | C | A | TTC39C | p.N500K | 0 | 60 | 0.74359 | 39 | WCM170_Z16_1 | Tissue |
| 19 | 18170889 | G | T | IL12RB1 | p.Q600K | 0 | 160 | 0.348 | 250 | WCM170_Z16_1 | Tissue |
| 19 | 58564971 | G | A | ZSCAN1 | p.R260H | 0 | 187 | 0.371951 | 164 | WCM170_Z16_1 | Tissue |
| 20 | 36767844 | C | G | TGM2 | p.D438H | 0 | 263 | 0.231707 | 492 | WCM170_Z16_1 | Tissue |
| 21 | 27102057 | C | T | ATP5J | p.V17I | 0 | 228 | 0.358621 | 145 | WCM170_Z16_1 | Tissue |
| 2 | 186656538 | C | T | FSIP2 | p.P1559S | 0 | 106 | 0.479167 | 48 | WCM170_Z16_1 | Tissue |
| 2 | 229890413 | C | T | PID1 | p.G230R | 0 | 70 | 0.429577 | 142 | WCM170_Z16_1 | Tissue |
| 2 | 29295516 | G | A | C2orf71 | p.Q538* | 0 | 51 | 0.053571 | 56 | WCM170_Z16_1 | Tissue |
| 3 | 127358327 | G | T | PODXL2 | p.E104* | 0 | 157 | 0.406417 | 187 | WCM170_Z16_1 | Tissue |
| 3 | 44684647 | G | C | ZNF197 | p.E675D | 0 | 44 | 0.390244 | 41 | WCM170_Z16_1 | Tissue |
| 5 | 52183702 | G | T | ITGA1 | p.V277F | 0 | 98 | 0.0625 | 48 | WCM170_Z16_1 | Tissue |
| 7 | 150695718 | C | A | NOS3 | p.Q256K | 0 | 221 | 0.354545 | 220 | WCM170_Z16_1 | Tissue |
| 7 | 57529622 | G | T | ZNF716 | p.M485I | 0 | 126 | 0.354839 | 124 | WCM170_Z16_1 | Tissue |
| 9 | 32633273 | C | T | TAF1L | p.A769T | 0 | 63 | 0.39726 | 73 | WCM170_Z16_1 | Tissue |
| 10 | 1038445 | C | A | GTPBP4 | p.T22K | 0 | 48 | 0.06383 | 47 | WCM172_X1_1 | Tissue |
| 1 | 11562050 | C | T | PTCHD2 | p.S334L | 0 | 90 | 0.148649 | 74 | WCM172_X1_1 | Tissue |
| 1 | 152127702 | C | T | RPTN | p.G625R | 0 | 110 | 0.275449 | 167 | WCM172_X1_1 | Tissue |
| 11 | 64806238 | G | A | SNX15 | p.A287T | 0 | 275 | 0.248062 | 258 | WCM172_X1_1 | Tissue |
| 12 | 109853384 | G | A | MYO1H | p.R513Q | 0 | 144 | 0.382199 | 191 | WCM172_X1_1 | Tissue |
| 1 | 224477260 | G | C | NVL | P.P501A | 0 | 62 | 0.102941 | 68 | WCM172_X1_1 | Tissue |
| 1 | 230904957 | C | T | CAPN9 | p.T246M | 0 | 88 | 0.521739 | 92 | WCM172_X1_1 | Tissue |
| 1 | 27684918 | C | T | MAP3K6 | p.R923Q | 0 | 170 | 0.296053 | 152 | WCM172_X1_1 | Tissue |
| 14 | 32903012 | G | A | AKAP6 | p.V105I | 0 | 99 | 0.235294 | 119 | WCM172_X1_1 | Tissue |
| 16 | 2134329 | G | A | TSC2 | p.R1369Q | 0 | 35 | 0.054545 | 55 | WCM172_X1_1 | Tissue |
| 16 | 9009316 | G | A | USP7 | p.R325C | 0 | 80 | 0.066667 | 90 | WCM172_X1_1 | Tissue |
| 19 | 49110516 | G | A | SPACA4 | p.R94H | 0 | 80 | 0.063492 | 63 | WCM172_X1_1 | Tissue |
| 2 | 32419022 | C | T | SLC30A6 | p.R156W | 0 | 47 | 0.065217 | 46 | WCM172_X1_1 | Tissue |
| 2 | 9607902 | T | G | CPSF3 | p.L618R | 0 | 111 | 0.058824 | 119 | WCM172_X1_1 | Tissue |
| 3 | 132363723 | G | T | ACAD11 | p.P56Q | 0 | 62 | 0.070423 | 71 | WCM172_X1_1 | Tissue |
| 3 | 44853755 | G | T | KIF15 | p.E751* | 0 | 246 | 0.272388 | 268 | WCM172_X1_1 | Tissue |
| 6 | 112462693 | A | C | LAMA4 | p.Y894D | 0 | 60 | 0.418605 | 43 | WCM172_X1_1 | Tissue |
| 6 | 33177715 | G | T | RING1 | p.R88L | 0 | 51 | 0.306452 | 62 | WCM172_X1_1 | Tissue |
| 6 | 42974348 | C | T | PPP2R5D | p.R85* | 0 | 130 | 0.21229 | 179 | WCM172_X1_1 | Tissue |
| 6 | 75912454 | A | C | COL12A1 | p.S19A | 0 | 179 | 0.364641 | 181 | WCM172_X1_1 | Tissue |
| 9 | 94486196 | G | T | ROR2 | p.S860R | 0 | 235 | 0.310714 | 280 | WCM172_X1_1 | Tissue |
| 10 | 38121401 | G | T | ZNF248 | p.D294E | 0 | 172 | 0.20202 | 99 | WCM172_X2_1 | Tissue |
| 1 | 11562050 | C | T | PTCHD2 | p.S334L | 0 | 90 | 0.371795 | 78 | WCM172_X2_1 | Tissue |
| 1 | 152127702 | C | T | RPTN | p.G625R | 0 | 110 | 0.581818 | 55 | WCM172_X2_1 | Tissue |
| 11 | 64806238 | G | A | SNX15 | p.A287T | 0 | 275 | 0.392265 | 181 | WCM172_X2_1 | Tissue |
| 11 | 76892428 | G | C | MYO7A | p.E899D | 0 | 208 | 0.070796 | 113 | WCM172_X2_1 | Tissue |
| 12 | 109853384 | G | A | MYO1H | p.R513Q | 0 | 144 | 0.420455 | 88 | WCM172_X2_1 | Tissue |
| 1 | 224477260 | G | C | NVL | p.P501A | 0 | 62 | 0.326531 | 49 | WCM172_X2_1 | Tissue |
| 1 | 230904957 | C | T | CAPN9 | p.T246M | 0 | 88 | 0.892473 | 93 | WCM172_X2_1 | Tissue |
| 12 | 49440489 | G | A | KMT2D | p.R1441C | 0 | 118 | 0.061224 | 49 | WCM172_X2_1 | Tissue |
| 12 | 57559911 | C | T | LRP1 | p.R906W | 0 | 58 | 0.070175 | 57 | WCM172_X2_1 | Tissue |
| 1 | 27684918 | C | T | MAP3K6 | p.R923Q | 0 | 170 | 0.561224 | 98 | WCM172_X2_1 | Tissue |
| 14 | 32903012 | G | A | AKAP6 | p.V105I | 0 | 99 | 0.333333 | 81 | WCM172_X2_1 | Tissue |
| 14 | 76437953 | A | T | TGFB3 | p.L154* | 0 | 31 | 0.091837 | 98 | WCM172_X2_1 | Tissue |
| 18 | 19116140 | C | T | ESCO1 | p.E684K | 0 | 39 | 0.066667 | 45 | WCM172_X2_1 | Tissue |
| 2 | 37513372 | C | G | PRKD3 | p.Q286H | 0 | 73 | 0.210526 | 76 | WCM172_X2_1 | Tissue |
| 3 | 44853755 | G | T | KIF15 | p.E751* | 0 | 246 | 0.320611 | 262 | WCM172_X2_1 | Tissue |
| 6 | 112462693 | A | C | LAMA4 | p.Y894D | 0 | 60 | 0.666667 | 36 | WCM172_X2_1 | Tissue |
| 6 | 136599171 | C | T | BCLAF1 | p.R283Q | 0 | 218 | 0.180645 | 155 | WCM172_X2_1 | Tissue |
| 6 | 33177715 | G | T | RING1 | p.R88L | 0 | 51 | 0.315789 | 76 | WCM172_X2_1 | Tissue |
| 6 | 42974348 | C | T | PPP2R5D | p.R85* | 0 | 130 | 0.363636 | 99 | WCM172_X2_1 | Tissue |
| 6 | 75912454 | A | C | COL12A1 | p.S19A | 0 | 179 | 0.430303 | 165 | WCM172_X2_1 | Tissue |
| 7 | 100682877 | C | A | MUC17 | p.A2727D | 0 | 232 | 0.069124 | 217 | WCM172_X2_1 | Tissue |
| 7 | 16815999 | T | A | TSPAN13 | p.F76Y | 0 | 68 | 0.072464 | 69 | WCM172_X2_1 | Tissue |
| 9 | 94486196 | G | T | ROR2 | p.S860R | 0 | 235 | 0.52459 | 305 | WCM172_X2_1 | Tissue |
| 11 | 108160489 | G | T | ATM | p.R1466L | 0 | 36 | 0.088889 | 45 | WCM176 | Plasma |
| 11 | 108224582 | A | G | ATM | p.T2921A | 0 | 44 | 0.061224 | 49 | WCM176 | Plasma |
| 1 | 161123830 | G | T | UFC1 | p.V15L | 0 | 50 | 0.096774 | 31 | WCM176 | Plasma |
| 12 | 11061365 | G | A | TAS2R13 | p.S178L | 0 | 42 | 0.085714 | 35 | WCM176 | Plasma |
| 14 | 93940602 | G | A | UNC79 | p.R104Q | 0 | 53 | 0.078947 | 38 | WCM176 | Plasma |
| 15 | 43712787 | T | C | TP53BP1 | p.D1461G | 0 | 35 | 0.371429 | 35 | WCM176 | Plasma |
| 15 | 43748441 | A | C | TP53BP1 | p.S784A | 0 | 39 | 0.075 | 40 | WCM176 | Plasma |
| 15 | 43749164 | T | C | TP53BP1 | p.T543A | 0 | 40 | 0.157895 | 38 | WCM176 | Plasma |
| 15 | 43749223 | C | G | TP53BP1 | p.S523T | 0 | 43 | 0.093023 | 43 | WCM176 | Plasma |
| 15 | 43749224 | T | C | TP53BP1 | p.S523G | 0 | 44 | 0.090909 | 44 | WCM176 | Plasma |
| 15 | 43769827 | T | C | TP53BP1 | p.T302A | 0 | 72 | 0.16 | 50 | WCM176 | Plasma |
| 15 | 43769897 | C | A | TP53BP1 | p.Q278H | 0 | 50 | 0.153846 | 52 | WCM176 | Plasma |
| 15 | 90129030 | G | T | TICRR | p.R423L | 0 | 35 | 0.078947 | 38 | WCM176 | Plasma |
| 17 | 73045382 | G | T | KCTD2 | p.R136L | 0 | 45 | 0.090909 | 33 | WCM176 | Plasma |
| 19 | 46916046 | C | T | CCDC8 | p.V8I | 0 | 35 | 0.081081 | 37 | WCM176 | Plasma |
| 22 | 28195045 | C | A | MN1 | p.G496V | 0 | 49 | 0.090909 | 33 | WCM176 | Plasma |
| 3 | 136035879 | G | T | PCCB | p.V355F | 0 | 58 | 0.071429 | 42 | WCM176 | Plasma |
| 3 | 172232713 | C | T | TNFSF10 | p.E70K | 0 | 33 | 0.071429 | 42 | WCM176 | Plasma |
| 5 | 140202861 | C | T | PCDHA5 | p.R501C | 0 | 67 | 0.088235 | 34 | WCM176 | Plasma |
| 9 | 78965788 | C | G | PCSK5 | p.L1644V | 0 | 42 | 0.096774 | 31 | WCM176 | Plasma |
| 11 | 66373322 | T | G | CCS | p.L274R | 0 | 211 | 0.35533 | 197 | WCM176_Z1_1 | Tissue |
| 12 | 131488739 | G | A | GPR133 | p.V385M | 0 | 64 | 0.08642 | 81 | WCM176_Z1_1 | Tissue |
| 12 | 65085348 | G | C | RASSF3 | p.E186Q | 0 | 134 | 0.361905 | 105 | WCM176_Z1_1 | Tissue |
| 15 | 75890986 | A | G | SNUPN | p.Y266H | 0 | 178 | 0.443662 | 142 | WCM176_Z1_1 | Tissue |
| 19 | 1428920 | G | A | DAZAP1 | p.R209Q | 0 | 198 | 0.542484 | 153 | WCM176_Z1_1 | Tissue |
| 22 | 24698332 | A | G | SPECC1L | p.T45A | 0 | 282 | 0.398524 | 271 | WCM176_Z1_1 | Tissue |
| 3 | 184144447 | C | T | RP11- | p.P37L | 0 | 69 | 0.416667 | 48 | WCM176_Z1_1 | Tissue |
| | | | | 433C9.2 | | | | | | | |
| 5 | 40959677 | C | T | C7 | p.T539M | 0 | 100 | 0.398305 | 118 | WCM176_Z1_1 | Tissue |
| 6 | 43410876 | G | A | ABCC10 | p.A799T | 0 | 83 | 0.392157 | 102 | WCM176_Z1_1 | Tissue |
| 9 | 134361525 | C | T | PRRC2B | p.R1938W | 0 | 414 | 0.41623 | 382 | WCM176_Z1_1 | Tissue |
| 10 | 103296387 | G | T | BTRC | p.R518S | 0 | 73 | 0.055046 | 109 | WCM183 | Plasma |
| 10 | 104174846 | C | T | PSD | p.D300N | 0 | 44 | 0.152174 | 92 | WCM183 | Plasma |
| 10 | 111860436 | G | A | ADD3 | p.V9M | 0 | 48 | 0.401869 | 107 | WCM183 | Plasma |
| 10 | 17373496 | T | G | ST8SIA6 | p.N145H | 0 | 62 | 0.067416 | 89 | WCM183 | Plasma |
| 10 | 48388191 | T | G | RBP3 | p.Q896P | 0 | 39 | 0.234375 | 64 | WCM183 | Plasma |
| 10 | 93244314 | T | G | HECTD2 | p.I291S | 0 | 69 | 0.121951 | 123 | WCM183 | Plasma |
| 11 | 1651119 | C | T | KRTAP5-5 | p.R17C | 0 | 43 | 0.055556 | 90 | WCM183 | Plasma |
| 11 | 46897468 | C | T | LRP4 | p.G1196R | 0 | 57 | 0.361111 | 108 | WCM183 | Plasma |
| 11 | 47370035 | G | A | MYBPC3 | p.R238C | 0 | 31 | 0.301205 | 83 | WCM183 | Plasma |
| 1 | 151239806 | A | G | PSMD4 | p.E374G | 0 | 42 | 0.173077 | 104 | WCM183 | Plasma |
| 1 | 151372091 | G | A | PSMB4 | p.G10R | 0 | 35 | 0.333333 | 51 | WCM183 | Plasma |
| 1 | 156916546 | C | G | ARHGEF11 | p.V828L | 0 | 37 | 0.305085 | 59 | WCM183 | Plasma |
| 11 | 62384796 | A | G | B3GAT3 | p.V94A | 0 | 40 | 0.22973 | 74 | WCM183 | Plasma |
| 11 | 69518088 | T | G | FGF19 | p.K93Q | 0 | 54 | 0.053191 | 94 | WCM183 | Plasma |
| 1 | 176526160 | T | A | PAPPA2 | p.S234R | 0 | 54 | 0.25 | 100 | WCM183 | Plasma |
| 11 | 94759539 | C | G | KDM4E | p.P273R | 0 | 37 | 0.058252 | 103 | WCM183 | Plasma |
| 12 | 110760817 | G | T | ATP2A2 | p.D162Y | 0 | 49 | 0.386139 | 101 | WCM183 | Plasma |
| 12 | 133398588 | C | T | GOLGA3 | p.V43I | 0 | 34 | 0.178571 | 84 | WCM183 | Plasma |
| 1 | 230827164 | G | A | COG2 | p.V657M | 0 | 35 | 0.356322 | 87 | WCM183 | Plasma |
| 1 | 235643456 | A | G | B3GALNT2 | p.F189L | 0 | 54 | 0.15 | 100 | WCM183 | Plasma |
| 1 | 237777529 | G | T | RYR2 | p.G1701C | 0 | 41 | 0.194915 | 118 | WCM183 | Plasma |
| 1 | 238050757 | C | G | ZP4 | p.D220H | 0 | 42 | 0.122449 | 98 | WCM183 | Plasma |
| 1 | 247059181 | G | C | AHCTF1 | p.S557C | 0 | 60 | 0.179245 | 106 | WCM183 | Plasma |
| 12 | 52162740 | A | G | SCN8A | p.Q998R | 0 | 50 | 0.121739 | 115 | WCM183 | Plasma |
| 12 | 57111005 | C | G | NACA | p.A1437P | 0 | 39 | 0.180328 | 61 | WCM183 | Plasma |
| 12 | 77424091 | C | G | E2F7 | p.K468N | 0 | 36 | 0.113402 | 97 | WCM183 | Plasma |
| 13 | 25275019 | G | A | ATP12A | p.V614M | 0 | 75 | 0.569231 | 130 | WCM183 | Plasma |
| 1 | 38155549 | T | C | C1orf109 | p.T2A | 0 | 42 | 0.272727 | 55 | WCM183 | Plasma |
| 1 | 45278702 | A | C | BTBD19 | p.E150A | 0 | 36 | 0.173077 | 52 | WCM183 | Plasma |
| 14 | 66208947 | G | A | FUT8 | p.R516Q | 0 | 64 | 0.642105 | 95 | WCM183 | Plasma |
| 14 | 73822431 | C | A | NUMB | p.R10I | 0 | 47 | 0.275362 | 69 | WCM183 | Plasma |
| 14 | 75248260 | A | C | YLPM1 | p.N505T | 0 | 40 | 0.128571 | 70 | WCM183 | Plasma |
| 14 | 75515587 | G | T | MLH3 | p.L258I | 0 | 39 | 0.053333 | 75 | WCM183 | Plasma |
| 14 | 75515589 | C | T | MLH3 | p.R257K | 0 | 40 | 0.054054 | 74 | WCM183 | Plasma |
| 14 | 77722884 | G | A | TMEM63C | p.V746M | 0 | 34 | 0.556962 | 79 | WCM183 | Plasma |
| 14 | 94582169 | C | T | IFI27 | p.A55V | 0 | 49 | 0.241379 | 87 | WCM183 | Plasma |
| 15 | 71184269 | G | T | THAP10 | p.L115I | 0 | 54 | 0.337209 | 86 | WCM183 | Plasma |
| 15 | 83456043 | G | C | FSD2 | p.H34D | 0 | 40 | 0.216495 | 97 | WCM183 | Plasma |
| 16 | 68289728 | T | C | PLA2G15 | p.Y188H | 0 | 45 | 0.378788 | 66 | WCM183 | Plasma |
| 16 | 9857926 | G | A | GRIN2A | p.R1159C | 0 | 61 | 0.255556 | 90 | WCM183 | Plasma |
| 1 | 70502205 | C | T | LRRC7 | p.S691L | 0 | 43 | 0.267606 | 71 | WCM183 | Plasma |
| 17 | 2275545 | G | A | SGSM2 | p.G502S | 0 | 84 | 0.32 | 125 | WCM183 | Plasma |
| 17 | 47696688 | T | C | SPOP | p.Y87C | 0 | 34 | 0.417722 | 79 | WCM183 | Plasma |
| 17 | 49270329 | C | A | MBTD1 | p.D502Y | 0 | 66 | 0.311927 | 109 | WCM183 | Plasma |
| 17 | 56332315 | C | A | LPO | p.L417M | 0 | 31 | 0.141304 | 92 | WCM183 | Plasma |
| 17 | 67092782 | A | T | ABCA6 | p.I1094N | 0 | 93 | 0.186207 | 145 | WCM183 | Plasma |
| 17 | 7133223 | C | G | DVL2 | p.R187P | 0 | 34 | 0.20339 | 59 | WCM183 | Plasma |
| 17 | 74163840 | C | A | RNF157 | p.S112I | 0 | 54 | 0.225 | 120 | WCM183 | Plasma |
| 17 | 76424614 | C | T | DNAH17 | p.E4189K | 0 | 31 | 0.272727 | 88 | WCM183 | Plasma |
| 17 | 8791542 | A | T | PIK3R5 | p.V521D | 0 | 58 | 0.289474 | 76 | WCM183 | Plasma |
| 18 | 210454 | T | C | USP14 | p.S432P | 0 | 54 | 0.214286 | 84 | WCM183 | Plasma |
| 18 | 51052995 | C | G | DCC | p.L1374V | 0 | 36 | 0.235294 | 85 | WCM183 | Plasma |
| 1 | 86909572 | C | T | CLCA2 | p.Q531* | 0 | 66 | 0.2 | 100 | WCM183 | Plasma |
| 19 | 12126943 | T | C | ZNF433 | p.T247A | 0 | 78 | 0.369748 | 119 | WCM183 | Plasma |
| 19 | 16006322 | C | T | CYP4F2 | p.A113T | 0 | 32 | 0.238095 | 84 | WCM183 | Plasma |
| 19 | 17379781 | G | A | BABAM1 | p.A56T | 0 | 81 | 0.346154 | 104 | WCM183 | Plasma |
| 19 | 5595407 | C | G | SAPB2 | p.Q628H | 0 | 40 | 0.213115 | 61 | WCM183 | Plasma |
| 19 | 56126233 | C | T | ZNF865 | p.R417C | 0 | 32 | 0.1875 | 32 | WCM183 | Plasma |
| 19 | 9068625 | C | T | MUC16 | p.R6274H | 0 | 56 | 0.333333 | 108 | WCM183 | Plasma |
| 20 | 168682 | C | G | DEFB128 | p.E43Q | 0 | 67 | 0.153846 | 130 | WCM183 | Plasma |
| 20 | 2297755 | G | C | TGM3 | p.W237C | 0 | 48 | 0.160377 | 106 | WCM183 | Plasma |
| 20 | 2845674 | C | G | VPS16 | p.Y659* | 0 | 56 | 0.153153 | 111 | WCM183 | Plasma |
| 20 | 35207220 | C | G | TGIF2 | p.L15V | 0 | 32 | 0.257576 | 66 | WCM183 | Plasma |
| 20 | 3683989 | G | T | SIGLEC1 | p.Y361* | 0 | 33 | 0.461538 | 91 | WCM183 | Plasma |
| 20 | 42223432 | C | A | IFT52 | p.L32I | 0 | 57 | 0.142857 | 105 | WCM183 | Plasma |
| 20 | 43090836 | C | A | C20orf62 | p.V68F | 0 | 32 | 0.283951 | 81 | WCM183 | Plasma |
| 21 | 19642347 | C | T | IMPRSS15 | p.R1000H | 0 | 68 | 0.240876 | 137 | WCM183 | Plasma |
| 21 | 19726056 | G | T | IMPRSS15 | p.N335K | 0 | 74 | 0.119403 | 134 | WCM183 | Plasma |
| 2 | 144460998 | C | A | ARHGAP15 | p.D383E | 0 | 43 | 0.051948 | 77 | WCM183 | Plasma |
| 2 | 160621143 | A | C | 7-Mar | p.N672H | 0 | 68 | 0.11215 | 107 | WCM183 | Plasma |
| 2 | 190557142 | C | G | ANKAR | p.Q401E | 0 | 50 | 0.445946 | 74 | WCM183 | Plasma |
| 2 | 198540059 | C | G | RFTN2 | p.D42H | 0 | 55 | 0.197802 | 91 | WCM183 | Plasma |
| 2 | 206036971 | G | C | PARD3B | p.V553L | 0 | 52 | 0.350515 | 97 | WCM183 | Plasma |
| 2 | 207615712 | G | T | MDH1B | p.A333D | 0 | 81 | 0.534483 | 116 | WCM183 | Plasma |
| 2 | 228882435 | C | G | SPHKAP | p.K1045N | 0 | 33 | 0.3 | 60 | WCM183 | Plasma |
| 2 | 27800725 | G | C | C2orf16 | p.G429A | 0 | 34 | 0.266667 | 105 | WCM183 | Plasma |
| 2 | 32693143 | C | T | BIRC6 | p.A1915V | 0 | 47 | 0.072727 | 110 | WCM183 | Plasma |
| 2 | 68364558 | C | T | WDR92 | p.S214N | 0 | 51 | 0.057143 | 105 | WCM183 | Plasma |
| 2 | 71744167 | C | T | DYSF | p.R302W | 0 | 58 | 0.539823 | 113 | WCM183 | Plasma |
| 2 | 99256375 | A | T | MGAT4A | p.H406Q | 0 | 31 | 0.1875 | 80 | WCM183 | Plasma |
| 3 | 118913124 | G | A | UPK1B | p.R176Q | 0 | 51 | 0.542056 | 107 | WCM183 | Plasma |
| 3 | 121187218 | A | T | POLQ | p.M2260K | 0 | 59 | 0.063636 | 110 | WCM183 | Plasma |
| 3 | 121212330 | G | T | POLQ | p.F839L | 0 | 89 | 0.172662 | 139 | WCM183 | Plasma |
| 3 | 121415555 | A | G | GOLGB1 | p.L1267S | 0 | 61 | 0.145631 | 103 | WCM183 | Plasma |
| 3 | 167039938 | T | C | ZBBX | p.Y317C | 0 | 59 | 0.234043 | 141 | WCM183 | Plasma |
| 3 | 190936699 | C | G | OSTN | p.S89C | 0 | 52 | 0.109375 | 128 | WCM183 | Plasma |
| 3 | 27465595 | T | C | SLC4A7 | p.K391R | 0 | 45 | 0.113636 | 88 | WCM183 | Plasma |
| 3 | 31725217 | C | A | OSBPL10 | p.E545D | 0 | 47 | 0.164948 | 97 | WCM183 | Plasma |
| 3 | 52821094 | A | C | ITIH1 | p.T623P | 0 | 35 | 0.315068 | 73 | WCM183 | Plasma |
| 3 | 93603590 | G | C | PROS1 | p.Q492E | 0 | 51 | 0.126126 | 111 | WCM183 | Plasma |
| 4 | 16597439 | C | T | LDB2 | p.D99N | 0 | 61 | 0.050505 | 99 | WCM183 | Plasma |
| 4 | 40824024 | G | C | APBB2 | p.S658C | 0 | 48 | 0.294737 | 95 | WCM183 | Plasma |
| 4 | 74283848 | C | T | ALB | p.T341M | 0 | 49 | 0.35 | 80 | WCM183 | Plasma |
| 5 | 148998522 | G | C | ARHGEF37 | p.D271H | 0 | 43 | 0.156627 | 83 | WCM183 | Plasma |
| 5 | 159996528 | T | A | ATP10B | p.T1305S | 0 | 47 | 0.295652 | 115 | WCM183 | Plasma |
| 5 | 35910017 | A | G | CAPSL | p.F159S | 0 | 65 | 0.184466 | 103 | WCM183 | Plasma |
| 5 | 76330271 | G | T | AGGF1 | p.R80L | 0 | 50 | 0.329787 | 94 | WCM183 | Plasma |
| 5 | 79837548 | T | C | FAM151B | p.L243P | 0 | 62 | 0.218182 | 110 | WCM183 | Plasma |
| 5 | 94253672 | C | T | MCTP1 | p.G427S | 0 | 41 | 0.279412 | 68 | WCM183 | Plasma |
| 6 | 102376391 | G | A | GRIK2 | p.A657T | 0 | 63 | 0.35 | 100 | WCM183 | Plasma |
| 6 | 121655438 | C | T | TBC1D32 | p.D47N | 0 | 67 | 0.150685 | 73 | WCM183 | Plasma |
| 6 | 129634205 | G | C | LAMA2 | p.C1125S | 0 | 35 | 0.108434 | 83 | WCM183 | Plasma |
| 6 | 152652697 | G | A | SYNE1 | p.P4375S | 0 | 44 | 0.181818 | 121 | WCM183 | Plasma |
| 6 | 56504349 | T | G | DST | p.T709P | 0 | 36 | 0.2 | 90 | WCM183 | Plasma |
| 6 | 7605635 | T | C | SNRNP48 | p.I241T | 0 | 53 | 0.1875 | 112 | WCM183 | Plasma |
| 7 | 100636331 | C | G | MUC12 | p.S972R | 0 | 77 | 0.144 | 125 | WCM183 | Plasma |
| 7 | 100678290 | G | C | MUC17 | p.S1198T | 0 | 49 | 0.19084 | 131 | WCM183 | Plasma |
| 7 | 107718670 | G | T | LAMB4 | p.S660Y | 0 | 43 | 0.277108 | 83 | WCM183 | Plasma |
| 7 | 117227838 | G | A | CFTR | p.G544S | 0 | 55 | 0.549451 | 91 | WCM183 | Plasma |
| 7 | 117235010 | C | A | CFTR | p.S839R | 0 | 39 | 0.306306 | 111 | WCM183 | Plasma |
| 7 | 142961754 | G | A | GSTK1 | p.V90M | 0 | 56 | 0.113043 | 115 | WCM183 | Plasma |
| 7 | 150553798 | C | G | AOC1 | p.Y80* | 0 | 56 | 0.396825 | 126 | WCM183 | Plasma |
| 8 | 100821613 | T | A | VPS13B | p.I2676N | 0 | 74 | 0.149701 | 167 | WCM183 | Plasma |
| 8 | 118541004 | A | G | MED30 | p.K98E | 0 | 77 | 0.051613 | 155 | WCM183 | Plasma |
| 8 | 124998311 | G | C | FER1L6 | p.V472L | 0 | 62 | 0.11194 | 134 | WCM183 | Plasma |
| 8 | 143846118 | T | A | LYNX1 | p.M101L | 0 | 54 | 0.113333 | 150 | WCM183 | Plasma |
| 8 | 146156387 | T | A | ZNF16 | p.T596S | 0 | 65 | 0.113333 | 150 | WCM183 | Plasma |
| 8 | 2857600 | C | A | CSMD1 | p.G2696C | 0 | 40 | 0.263158 | 57 | WCM183 | Plasma |
| 9 | 109689821 | A | T | ZNF462 | p.I1210F | 0 | 47 | 0.206897 | 87 | WCM183 | Plasma |
| 9 | 116930056 | T | G | COL27A1 | p.F74C | 0 | 60 | 0.17037 | 135 | WCM183 | Plasma |
| 9 | 124929180 | G | A | MORN5 | p.G61R | 0 | 31 | 0.311828 | 93 | WCM183 | Plasma |
| 9 | 95078261 | G | A | NOL8 | p.P216S | 0 | 52 | 0.459854 | 137 | WCM183 | Plasma |
| 9 | 98221932 | G | A | PTCH1 | p.P946L | 0 | 56 | 0.231481 | 108 | WCM183 | Plasma |
| 9 | 98643423 | A | C | ERCC6L2 | p.K118Q | 0 | 59 | 0.565574 | 122 | WCM183 | Plasma |
| X | 26212711 | G | A | MAGEB6 | p.V250I | 0 | 51 | 0.578125 | 64 | WCM183 | Plasma |
| 10 | 103919727 | G | T | NOLC1 | p.A312S | 0 | 85 | 0.155844 | 77 | WCM183_Z4_1 | Tissue |
| 10 | 104174846 | C | T | PSD | p.D300N | 0.008333 | 120 | 0.23741 | 139 | WCM183_Z4_1 | Tissue |
| 10 | 1230887 | C | T | ADARB2 | p.A653T | 0 | 155 | 0.5 | 90 | WCM183_Z4_1 | Tissue |
| 10 | 48388191 | T | G | RBP3 | p.Q896P | 0 | 128 | 0.457627 | 59 | WCM183_Z4_1 | Tissue |
| 10 | 73572594 | G | C | CDH23 | p.E3199Q | 0 | 194 | 0.195122 | 205 | WCM183_Z4_1 | Tissue |
| 11 | 46897468 | C | T | LRP4 | p.G1196R | 0 | 206 | 0.46087 | 115 | WCM183_Z4_1 | Tissue |
| 11 | 47370035 | G | A | MYBPC3 | p.R238C | 0 | 96 | 0.2625 | 80 | WCM183_Z4_1 | Tissue |
| 1 | 151239806 | A | G | PSMD4 | p.E374G | 0 | 283 | 0.238095 | 252 | WCM183_Z4_1 | Tissue |
| 1 | 151372091 | G | A | PSMB4 | p.G10R | 0 | 126 | 0.587156 | 109 | WCM183_Z4_1 | Tissue |
| 11 | 63059031 | A | G | SLC22A10 | p.Y141C | 0 | 71 | 0.204082 | 98 | WCM183_Z4_1 | Tissue |
| 11 | 64121627 | G | A | CCDC88B | p.E1362K | 0 | 169 | 0.318182 | 220 | WCM183_Z4_1 | Tissue |
| 1 | 176526160 | T | A | PAPPA2 | p.S234R | 0 | 69 | 0.194444 | 108 | WCM183_Z4_1 | Tissue |
| 12 | 110760817 | G | T | ATP2A2 | p.D162Y | 0 | 141 | 0.194286 | 175 | WCM183_Z4_1 | Tissue |
| 12 | 113826415 | A | G | PLBD2 | p.K585R | 0 | 201 | 0.789474 | 190 | WCM183_Z4_1 | Tissue |
| 12 | 122750898 | G | T | VPS33A | p.R20S | 0 | 52 | 0.06 | 50 | WCM183_Z4_1 | Tissue |
| 12 | 133398588 | C | T | GOLGA3 | p.V43I | 0 | 55 | 0.265306 | 49 | WCM183_Z4_1 | Tissue |
| 1 | 230827164 | G | A | COG2 | p.V657M | 0 | 53 | 0.480769 | 52 | WCM183_Z4_1 | Tissue |
| 12 | 32134263 | G | C | KIAA1551 | p.R125T | 0 | 141 | 0.192308 | 130 | WCM183_Z4_1 | Tissue |
| 1 | 237777529 | G | T | RYR2 | p.G1701C | 0 | 216 | 0.269663 | 267 | WCM183_Z4_1 | Tissue |
| 1 | 238050757 | C | G | ZP4 | p.D220H | 0.009346 | 107 | 0.188976 | 127 | WCM183_Z4_1 | Tissue |
| 1 | 247059181 | G | C | AHCTF1 | p.S557C | 0 | 56 | 0.113636 | 44 | WCM183_Z4_1 | Tissue |
| 12 | 52162740 | A | G | SCN8A | p.Q998R | 0 | 355 | 0.226257 | 358 | WCM183_Z4_1 | Tissue |
| 12 | 57111005 | C | G | NACA | p.A1437P | 0 | 154 | 0.165714 | 175 | WCM183_Z4_1 | Tissue |
| 12 | 57619421 | C | T | NXPH4 | p.P273L | 0 | 107 | 0.346154 | 78 | WCM183_Z4_1 | Tissue |
| 12 | 66786226 | C | G | GRIP1 | p.G724R | 0 | 171 | 0.266304 | 184 | WCM183_Z4_1 | Tissue |
| 12 | 69090622 | T | G | NUP107 | p.F158V | 0 | 157 | 0.225806 | 124 | WCM183_Z4_1 | Tissue |
| 12 | 7354909 | C | T | PEX5 | p.Q239* | 0 | 133 | 0.158333 | 120 | WCM183_Z4_1 | Tissue |
| 12 | 77424091 | C | G | E2F7 | p.K468N | 0 | 50 | 0.353846 | 65 | WCM183_Z4_1 | Tissue |
| 13 | 25275019 | G | A | ATP12A | p.V614M | 0 | 111 | 0.509804 | 102 | WCM183_Z4_1 | Tissue |
| 14 | 27064727 | G | A | NOVA1 | p.P57S | 0 | 97 | 0.136 | 125 | WCM183_Z4_1 | Tissue |
| 14 | 33147602 | G | T | AKAP6 | p.S939I | 0 | 52 | 0.06 | 50 | WCM183_Z4_1 | Tissue |
| 14 | 66208947 | G | A | FUT8 | p.R516Q | 0 | 145 | 0.712963 | 108 | WCM183_Z4_1 | Tissue |
| 14 | 70925489 | G | A | ADAM21 | p.V425I | 0 | 53 | 0.333333 | 36 | WCM183_Z4_1 | Tissue |
| 14 | 73822431 | C | A | NUMB | p.R10I | 0 | 82 | 0.481481 | 54 | WCM183_Z4_1 | Tissue |
| 14 | 75248260 | A | C | YLPM1 | p.N505T | 0 | 86 | 0.160714 | 56 | WCM183_Z4_1 | Tissue |
| 15 | 71184269 | G | T | THAP10 | p.L115I | 0 | 81 | 0.947368 | 57 | WCM183_Z4_1 | Tissue |
| 16 | 20360281 | C | A | UMOD | p.E114D | 0 | 46 | 0.266667 | 45 | WCM183_Z4_1 | Tissue |
| 16 | 66919683 | C | T | PDP2 | p.P499L | 0 | 107 | 0.357143 | 56 | WCM183_Z4_1 | Tissue |
| 16 | 68289728 | T | C | PLA2G15 | p.Y188H | 0 | 219 | 0.471264 | 87 | WCM183_Z4_1 | Tissue |
| 16 | 74640905 | G | C | GLG1 | p.L30V | 0 | 70 | 0.297297 | 37 | WCM183_Z4_1 | Tissue |
| 16 | 9857926 | G | A | GRIN2A | p.R1159C | 0 | 43 | 0.235294 | 34 | WCM183_Z4_1 | Tissue |
| 17 | 28530317 | C | T | SLC6A4 | p.R564Q | 0 | 52 | 0.149254 | 67 | WCM183_Z4_1 | Tissue |
| 17 | 33495121 | C | A | UNC45B | p.A398D | 0 | 71 | 0.428571 | 49 | WCM183_Z4_1 | Tissue |
| 17 | 4449158 | C | G | MYBBP1A | p.S635T | 0 | 179 | 0.254386 | 114 | WCM183_Z4_1 | Tissue |
| 17 | 47696688 | T | C | SPOP | p.Y87C | 0 | 40 | 0.30303 | 66 | WCM183_Z4_1 | Tissue |
| 17 | 49270329 | C | A | MBTD1 | p.D502Y | 0 | 147 | 0.608108 | 148 | WCM183_Z4_1 | Tissue |
| 17 | 56332315 | C | A | LPO | p.L417M | 0 | 127 | 0.196078 | 153 | WCM183_Z4_1 | Tissue |
| 17 | 62553818 | G | C | SMURF2 | p.H447D | 0 | 93 | 0.124031 | 129 | WCM183_Z4_1 | Tissue |
| 17 | 74382357 | C | A | SPHK1 | p.D73E | 0 | 108 | 0.495495 | 111 | WCM183_Z4_1 | Tissue |
| 17 | 76455115 | C | A | DNAH17 | p.E3272* | 0 | 36 | 0.53125 | 32 | WCM183_Z4_1 | Tissue |
| 1 | 86909572 | C | T | CLCA2 | p.Q531* | 0 | 106 | 0.411765 | 51 | WCM183_Z4_1 | Tissue |
| 19 | 16006322 | C | T | CYP4F2 | p.A113T | 0 | 78 | 0.142857 | 63 | WCM183_Z4_1 | Tissue |
| 19 | 17379781 | G | A | BABAM1 | p.A56T | 0 | 115 | 0.146789 | 109 | WCM183_Z4_1 | Tissue |
| 19 | 36051436 | C | A | ATP4A | p.G206C | 0 | 129 | 0.214286 | 126 | WCM183_Z4_1 | Tissue |
| 19 | 36351555 | C | T | KIRREL2 | p.A305V | 0 | 131 | 0.206667 | 150 | WCM183_Z4_1 | Tissue |
| 19 | 48806005 | G | A | CCDC114 | p.R359C | 0 | 156 | 0.235714 | 140 | WCM183_Z4_1 | Tissue |
| 19 | 52658531 | C | A | ZNF836 | p.G802V | 0 | 71 | 0.372093 | 43 | WCM183_Z4_1 | Tissue |
| 19 | 55696926 | C | G | PTPRH | p.M1002I | 0 | 318 | 0.257812 | 256 | WCM183_Z4_1 | Tissue |
| 19 | 7509218 | G | C | ARHGEF18 | p.D309H | 0 | 31 | 0.25 | 32 | WCM183_Z4_1 | Tissue |
| 19 | 9019996 | A | G | MUC16 | p.L12500P | 0 | 116 | 0.207921 | 101 | WCM183_Z4_1 | Tissue |
| 19 | 9068625 | C | T | MUC16 | p.R6274H | 0 | 209 | 0.242236 | 161 | WCM183_Z4_1 | Tissue |
| 20 | 168682 | C | G | DEFB128 | p.E43Q | 0 | 43 | 0.166667 | 36 | WCM183_Z4_1 | Tissue |
| 20 | 2297755 | G | C | TGM3 | p.W237C | 0 | 69 | 0.130435 | 69 | WCM183_Z4_1 | Tissue |
| 20 | 35207220 | C | G | TGIF2 | p.L15V | 0 | 58 | 0.489583 | 96 | WCM183_Z4_1 | Tissue |
| 20 | 3683989 | G | T | SIGLEC1 | p.Y361* | 0 | 213 | 0.393782 | 193 | WCM183_Z4_1 | Tissue |
| 20 | 37182550 | A | G | RALGAPB | p.Q1068R | 0 | 80 | 0.179487 | 78 | WCM183_Z4_1 | Tissue |
| 20 | 42223432 | C | A | IFT52 | p.L32I | 0 | 140 | 0.094488 | 127 | WCM183_Z4_1 | Tissue |
| 20 | 43090836 | C | A | C20orf62 | p.V68F | 0 | 58 | 0.22449 | 49 | WCM183_Z4_1 | Tissue |
| 20 | 4680111 | G | T | PRNP | p.G82V | 0 | 68 | 0.070175 | 57 | WCM183_Z4_1 | Tissue |
| 20 | 61288042 | C | T | SLCO4A1 | p.S79L | 0 | 59 | 0.235294 | 68 | WCM183_Z4_1 | Tissue |
| 2 | 160621143 | A | C | 7-Mar | p.N672H | 0 | 74 | 0.215686 | 51 | WCM183_Z4_1 | Tissue |
| 2 | 1795677 | C | T | MYT1L | p.E1175K | 0 | 77 | 0.155556 | 45 | WCM183_Z4_1 | Tissue |
| 2 | 1795679 | A | G | MYT1L | p.L1174P | 0 | 77 | 0.155556 | 45 | WCM183_Z4_1 | Tissue |
| 2 | 190557142 | C | G | ANKAR | p.Q401E | 0 | 120 | 0.320988 | 81 | WCM183_Z4_1 | Tissue |
| 2 | 197171902 | C | A | HECW2 | p.A881S | 0 | 53 | 0.2 | 60 | WCM183_Z4_1 | Tissue |
| 2 | 206036971 | G | C | PARD3B | p.V553L | 0 | 67 | 0.181818 | 66 | WCM183_Z4_1 | Tissue |
| 2 | 207615712 | G | T | MDH1B | p.A333D | 0 | 89 | 0.491071 | 112 | WCM183_Z4_1 | Tissue |
| 22 | 26348303 | C | T | MYO18B | p.R1963* | 0 | 179 | 0.254658 | 161 | WCM183_Z4_1 | Tissue |
| 22 | 26688483 | C | T | SEZ6L | p.A69V | 0 | 47 | 0.183673 | 49 | WCM183_Z4_1 | Tissue |
| 22 | 46792553 | C | T | CELSR1 | p.G1931D | 0 | 105 | 0.306667 | 75 | WCM183_Z4_1 | Tissue |
| 2 | 32693143 | C | T | BIRC6 | p.A1915V | 0 | 65 | 0.236842 | 76 | WCM183_Z4_1 | Tissue |
| 2 | 71744167 | C | T | DYSF | p.R302W | 0 | 210 | 0.554054 | 222 | WCM183_Z4_1 | Tissue |
| 3 | 121415555 | A | G | GOLGB1 | p.L1267S | 0 | 45 | 0.25 | 36 | WCM183_Z4_1 | Tissue |
| 3 | 93603590 | G | C | PROS1 | p.Q492E | 0 | 70 | 0.258621 | 58 | WCM183_Z4_1 | Tissue |
| 4 | 134073075 | G | T | PCDH10 | p.A594S | 0 | 31 | 0.648649 | 37 | WCM183_Z4_1 | Tissue |
| 4 | 40824024 | G | C | APBB2 | p.S658C | 0 | 71 | 0.36 | 50 | WCM183_Z4_1 | Tissue |
| 4 | 53525295 | G | T | USP46 | p.S8Y | 0 | 117 | 0.183099 | 142 | WCM183_Z4_1 | Tissue |
| 4 | 71099764 | G | T | FDCSP | p.G40W | 0 | 66 | 0.445946 | 74 | WCM183_Z4_1 | Tissue |
| 4 | 74283848 | C | T | ALB | p.T341M | 0 | 101 | 0.418367 | 98 | WCM183_Z4_1 | Tissue |
| 5 | 1293872 | G | A | TERT | p.R377C | 0 | 72 | 0.111111 | 36 | WCM183_Z4_1 | Tissue |
| 5 | 137727446 | G | A | KDM3B | p.A709T | 0 | 144 | 0.463415 | 123 | WCM183_Z4_1 | Tissue |
| 5 | 159996528 | T | A | ATP10B | p.T1305S | 0 | 188 | 0.232068 | 237 | WCM183_Z4_1 | Tissue |
| 5 | 34808752 | A | T | RAI14 | p.K148I | 0 | 228 | 0.257353 | 272 | WCM183_Z4_1 | Tissue |
| 5 | 35910017 | A | G | CAPSL | p.F159S | 0 | 35 | 0.176471 | 34 | WCM183_Z4_1 | Tissue |
| 5 | 76342409 | G | T | AGGF1 | p.E370* | 0 | 138 | 0.554622 | 119 | WCM183_Z4_1 | Tissue |
| 5 | 79837548 | T | C | FAM151B | p.L243P | 0 | 85 | 0.35 | 40 | WCM183_Z4_1 | Tissue |
| 6 | 121655438 | C | T | TBC1D32 | p.D47N | 0 | 137 | 0.181818 | 143 | WCM183_Z4_1 | Tissue |
| 6 | 129634205 | G | C | LAMA2 | p.C1125S | 0 | 255 | 0.190476 | 252 | WCM183_Z4_1 | Tissue |
| 6 | 46518129 | G | A | CYP39A1 | p.R462* | 0 | 86 | 0.124031 | 129 | WCM183_Z4_1 | Tissue |
| 7 | 100636331 | C | G | MUC12 | p.S972R | 0 | 128 | 0.167883 | 137 | WCM183_Z4_1 | Tissue |
| 7 | 100678290 | G | C | MUC17 | p.S1198T | 0 | 171 | 0.260606 | 165 | WCM183_Z4_1 | Tissue |
| 7 | 107718670 | G | T | LAMB4 | p.S660Y | 0 | 124 | 0.373832 | 107 | WCM183_Z4_1 | Tissue |
| 7 | 117235010 | C | A | CFTR | p.S839R | 0 | 151 | 0.234043 | 94 | WCM183_Z4_1 | Tissue |
| 7 | 142961754 | G | A | GSTK1 | p.V90M | 0 | 147 | 0.314286 | 105 | WCM183_Z4_1 | Tissue |
| 7 | 150553798 | C | G | AOC1 | p.Y80* | 0 | 183 | 0.276923 | 130 | WCM183_Z4_1 | Tissue |
| 7 | 50680445 | A | T | GRB10 | p.L396H | 0 | 192 | 0.124498 | 249 | WCM183_Z4_1 | Tissue |
| 7 | 88966340 | G | C | ZNF804B | p.L1348F | 0 | 37 | 0.179104 | 67 | WCM183_Z4_1 | Tissue |
| 8 | 118541004 | A | G | MED30 | p.K98E | 0 | 48 | 0.116883 | 154 | WCM183_Z4_1 | Tissue |
| 8 | 2857600 | C | A | CSMD1 | p.G2696C | 0 | 133 | 0.609756 | 123 | WCM183_Z4_1 | Tissue |
| 9 | 109689821 | A | T | ZNF462 | p.I1210F | 0 | 144 | 0.187879 | 165 | WCM183_Z4_1 | Tissue |
| 9 | 116930056 | T | G | COL27A1 | p.F74C | 0 | 236 | 0.090361 | 166 | WCM183_Z4_1 | Tissue |
| 9 | 124929180 | G | A | MORN5 | p.G61R | 0 | 51 | 0.440678 | 59 | WCM183_Z4_1 | Tissue |
| 9 | 135277689 | T | G | TTF1 | p.K174Q | 0 | 43 | 0.659091 | 44 | WCM183_Z4_1 | Tissue |
| 9 | 6007411 | C | A | KIAA2026 | p.G126V | 0 | 55 | 0.09375 | 32 | WCM183_Z4_1 | Tissue |
| X | 26212711 | G | A | MAGEB6 | p.V250I | 0 | 98 | 0.923077 | 39 | WCM183_Z4_1 | Tissue |
| 1 | 103412502 | C | G | COL11A1 | p.G1060A | 0 | 33 | 0.060403 | 149 | WCM185 | Plasma |
| 1 | 103412502 | C | G | COL11A1 | p.G1060A | 0 | 33 | 0.1 | 100 | WCM185-TP1 | Plasma |
| 15 | 53049924 | C | T | ONECUT1 | p.R409H | 0 | 32 | 0.211538 | 52 | WCM185-TP1 | Plasma |
| 2 | 96557455 | T | A | ANKRD36C | p.N939Y | 0 | 44 | 0.078947 | 152 | WCM185-TP1 | Plasma |
| 8 | 91837017 | A | G | NECAB1 | p.H66R | 0 | 36 | 0.2 | 100 | WCM185-TP1 | Plasma |
| 1 | 103412502 | C | G | COL11A1 | p.G1060A | 0 | 33 | 0.368852 | 122 | WCM185-TP2 | Plasma |
| 1 | 12819361 | G | T | C1orf158 | p.R115L | 0 | 34 | 0.086957 | 46 | WCM185-TP2 | Plasma |
| 11 | 85418516 | C | T | SYTL2 | p.V687I | 0 | 39 | 0.256637 | 113 | WCM185-TP2 | Plasma |
| 15 | 53049924 | C | T | ONECUT1 | p.R409H | 0 | 32 | 0.254545 | 55 | WCM185-TP2 | Plasma |
| 2 | 96557455 | T | A | ANKRD36C | p.N939Y | 0 | 44 | 0.063694 | 157 | WCM185-TP2 | Plasma |
| 8 | 91837017 | A | G | NECAB1 | p.H66R | 0 | 36 | 0.267857 | 112 | WCM185-TP2 | Plasma |
| 1 | 103412502 | C | G | COL11A1 | p.G1060A | 0 | 33 | 0.401869 | 107 | WCM185-TP3 | Plasma |
| 11 | 85418516 | C | T | SYTL2 | p.V687I | 0 | 39 | 0.230769 | 117 | WCM185-TP3 | Plasma |
| 15 | 53049924 | C | T | ONECUT1 | p.R409H | 0 | 32 | 0.326923 | 52 | WCM185-TP3 | Plasma |
| 2 | 96557455 | T | A | ANKRD36C | p.N939Y | 0 | 44 | 0.070423 | 142 | WCM185-TP3 | Plasma |
| 8 | 91837017 | A | G | NECAB1 | p.H66R | 0 | 36 | 0.301075 | 93 | WCM185-TP3 | Plasma |
| 15 | 53049924 | C | T | ONECUT1 | p.R409H | 0 | 32 | 0.104478 | 67 | WCM185-TP4 | Plasma |
| 2 | 96557455 | T | A | ANKRD36C | p.N939Y | 0 | 44 | 0.077922 | 154 | WCM185-TP4 | Plasma |
| 4 | 169189013 | G | T | DDX60 | p.Q970K | 0 | 34 | 0.052632 | 76 | WCM185-TP4 | Plasma |
| 10 | 108371773 | G | A | SORCS1 | p.R977W | 0 | 99 | 0.084507 | 71 | WCM185_X1_1 | Tissue |
| 10 | 87966267 | C | A | GRID1 | p.R125L | 0 | 31 | 0.081081 | 37 | WCM185_X1_1 | Tissue |
| 11 | 31327201 | T | C | DCDC1 | p.T239A | 0 | 151 | 0.081967 | 122 | WCM185_X1_1 | Tissue |
| 1 | 156914912 | C | T | ARHGEF11 | p.V924I | 0 | 37 | 0.151515 | 33 | WCM185_X1_1 | Tissue |
| 11 | 64335269 | C | A | SLC22A11 | p.N419K | 0 | 103 | 0.098361 | 61 | WCM185_X1_1 | Tissue |
| 1 | 205129365 | C | A | DSTYK | p.G661V | 0 | 82 | 0.076923 | 78 | WCM185_X1_1 | Tissue |
| 12 | 130648167 | C | A | FZD10 | p.A227E | 0 | 74 | 0.096774 | 62 | WCM185_X1_1 | Tissue |
| 15 | 53049924 | C | T | ONECUT1 | p.R409H | 0 | 75 | 0.129032 | 31 | WCM185_X1_1 | Tissue |
| 16 | 75688215 | G | T | TERF2IP | p.V239L | 0 | 51 | 0.068182 | 44 | WCM185_X1_1 | Tissue |
| 17 | 11572819 | C | T | DNAH9 | p.R1021W | 0 | 121 | 0.065217 | 46 | WCM185_X1_1 | Tissue |
| 17 | 47696643 | A | C | SPOP | p.F102C | 0 | 96 | 0.072727 | 110 | WCM185_X1_1 | Tissue |
| 19 | 52919239 | G | T | ZNF528 | p.R378S | 0 | 68 | 0.130435 | 46 | WCM185_X1_1 | Tissue |
| 2 | 172928495 | C | A | METAP1D | p.S85R | 0 | 77 | 0.065217 | 46 | WCM185_X1_1 | Tissue |
| 2 | 176958096 | G | A | HOXD13 | p.G160R | 0 | 88 | 0.072165 | 97 | WCM185_X1_1 | Tissue |
| 2 | 71650878 | G | T | ZNF638 | p.E1412* | 0 | 56 | 0.088889 | 45 | WCM185_X1_1 | Tissue |
| 4 | 8442605 | G | T | TRMT44 | p.G19V | 0 | 109 | 0.057971 | 138 | WCM185_X1_1 | Tissue |
| 5 | 113698689 | C | A | KCNN2 | p.H73N | 0 | 62 | 0.086957 | 69 | WCM185_X1_1 | Tissue |
| 6 | 24718813 | G | T | C6orf62 | p.D28E | 0 | 80 | 0.06383 | 47 | WCM185_X1_1 | Tissue |
| 7 | 102079481 | C | A | ORAI2 | p.Y26* | 0 | 52 | 0.096774 | 31 | WCM185_X1_1 | Tissue |
| 9 | 139889462 | C | T | CLIC3 | p.E158K | 0 | 71 | 0.125 | 32 | WCM185_X1_1 | Tissue |
| X | 12903734 | G | T | TLR7 | p.C36F | 0 | 121 | 0.125 | 48 | WCM185_X1_1 | Tissue |
| 10 | 25887500 | A | G | GPR158 | p.N982S | 0 | 232 | 0.403433 | 233 | WCM185_Z1_1 | Tissue |
| 1 | 103412502 | C | G | COL11A1 | p.G1060A | 0 | 85 | 0.368794 | 141 | WCM185_Z1_1 | Tissue |
| 1 | 208207879 | G | T | PLXNA2 | p.P1608H | 0 | 41 | 0.061224 | 49 | WCM185_Z1_1 | Tissue |
| 15 | 32460211 | G | A | CHRNA7 | p.R354H | 0 | 35 | 0.354839 | 31 | WCM185_Z1_1 | Tissue |
| 15 | 53049924 | C | T | ONECUT1 | p.R409H | 0 | 75 | 0.397959 | 98 | WCM185_Z1_1 | Tissue |
| 15 | 70345681 | C | G | TLE3 | p.C623S | 0 | 88 | 0.39 | 100 | WCM185_Z1_1 | Tissue |
| 17 | 47696643 | A | C | SPOP | p.F102C | 0 | 96 | 0.408 | 125 | WCM185_Z1_1 | Tissue |
| 22 | 32012774 | C | T | SFI1 | p.P1115L | 0 | 121 | 0.386364 | 176 | WCM185_Z1_1 | Tissue |
| 2 | 71650878 | G | T | ZNF638 | p.E1412* | 0 | 56 | 0.344262 | 61 | WCM185_Z1_1 | Tissue |
| 5 | 82833515 | G | A | VCAN | p.A1565T | 0 | 106 | 0.318966 | 116 | WCM185_Z1_1 | Tissue |
| 6 | 53519871 | C | T | KLHL31 | p.R67Q | 0 | 206 | 0.294964 | 278 | WCM185_Z1_1 | Tissue |
| 8 | 67926691 | T | G | PPP1R42 | p.N89T | 0 | 37 | 0.21875 | 32 | WCM185_Z1_1 | Tissue |
| 8 | 91837017 | A | G | NECAB1 | p.H66R | 0 | 87 | 0.344828 | 58 | WCM185_Z1_1 | Tissue |
| 15 | 72645482 | C | T | HEXA | p.R166H | 0 | 44 | 0.097561 | 82 | WCM192 | Plasma |
| 20 | 9624840 | G | A | PAK7 | p.T46M | 0 | 34 | 0.051948 | 77 | WCM192 | Plasma |
| 2 | 190660510 | G | A | PMS1 | p.D50N | 0 | 41 | 0.084337 | 83 | WCM192 | Plasma |
| 6 | 20212428 | C | T | MBOAT1 | p.R13H | 0 | 37 | 0.053333 | 75 | WCM192 | Plasma |
| 9 | 94172733 | C | T | NFIL3 | p.R95Q | 0 | 42 | 0.136364 | 66 | WCM192 | Plasma |
| 10 | 100190955 | G | A | HPS1 | p.R201W | 0 | 86 | 0.169492 | 59 | WCM192_X1_1 | Tissue |
| 10 | 128193341 | G | A | C10orf90 | p.T143M | 0 | 116 | 0.054545 | 110 | WCM192_X1_1 | Tissue |
| 10 | 22498209 | A | G | EBLN1 | p.M235T | 0 | 105 | 0.102564 | 39 | WCM192_X1_1 | Tissue |
| 10 | 22498468 | C | T | EBLN1 | p.G149R | 0 | 87 | 0.065217 | 92 | WCM192_X1_1 | Tissue |
| 10 | 97444321 | C | T | TCTN3 | p.G344R | 0 | 92 | 0.109589 | 73 | WCM192_X1_1 | Tissue |
| 10 | 97453433 | G | A | TCTN3 | p.A75V | 0 | 52 | 0.093023 | 43 | WCM192_X1_1 | Tissue |
| 11 | 1080487 | G | A | MUC2 | p.A377T | 0 | 122 | 0.068063 | 191 | WCM192_X1_1 | Tissue |
| 11 | 129245968 | G | T | BARX2 | p.G13V | 0 | 62 | 0.358209 | 67 | WCM192_X1_1 | Tissue |
| 1 | 119927488 | C | G | HAO2 | p.R125G | 0 | 118 | 0.294118 | 34 | WCM192_X1_1 | Tissue |
| 1 | 1361641 | C | T | TMEM88B | p.P45L | 0 | 32 | 0.173077 | 52 | WCM192_X1_1 | Tissue |
| 11 | 4608076 | C | T | OR52I2 | p.L12F | 0 | 35 | 0.175 | 40 | WCM192_X1_1 | Tissue |
| 1 | 1500277 | G | A | SSU72 | p.R34* | 0 | 54 | 0.516129 | 31 | WCM192_X1_1 | Tissue |
| 1 | 151140732 | G | A | SCNM1 | p.E171K | 0 | 149 | 0.105263 | 152 | WCM192_X1_1 | Tissue |
| 1 | 156567904 | G | A | GPATCH4 | p.Q91* | 0 | 142 | 0.387097 | 93 | WCM192_X1_1 | Tissue |
| 1 | 161145823 | A | G | B4GALT3 | p.C10R | 0 | 154 | 0.066667 | 225 | WCM192_X1_1 | Tissue |
| 11 | 8662516 | T | C | TRIM66 | p.H324R | 0 | 64 | 0.1 | 40 | WCM192_X1_1 | Tissue |
| 11 | 9087449 | C | T | SCUBE2 | p.R280Q | 0 | 74 | 0.088235 | 34 | WCM192_X1_1 | Tissue |
| 1 | 201176651 | C | T | IGFN1 | p.T877M | 0 | 252 | 0.053628 | 317 | WCM192_X1_1 | Tissue |
| 1 | 201181994 | A | G | IGFN1 | p.D2658G | 0 | 364 | 0.077406 | 478 | WCM192_X1_1 | Tissue |
| 1 | 202097559 | G | A | GPR37L1 | p.G441R | 0 | 64 | 0.115385 | 52 | WCM192_X1_1 | Tissue |
| 1 | 20417135 | C | G | PLA2G5 | p.R123G | 0 | 173 | 0.067633 | 207 | WCM192_X1_1 | Tissue |
| 1 | 211956765 | C | T | LPGAT1 | p.W178* | 0 | 59 | 0.068493 | 73 | WCM192_X1_1 | Tissue |
| 12 | 22487045 | C | T | ST8SIA1 | p.C41Y | 0 | 203 | 0.089286 | 112 | WCM192_X1_1 | Tissue |
| 1 | 23280424 | G | T | LACTBL1 | p.N251K | 0 | 50 | 0.244444 | 45 | WCM192_X1_1 | Tissue |
| 1 | 232941142 | G | A | MAP10 | p.G125S | 0 | 120 | 0.057471 | 87 | WCM192_X1_1 | Tissue |
| 1 | 24718079 | C | A | STPG1 | p.S54I | 0 | 81 | 0.06 | 150 | WCM192_X1_1 | Tissue |
| 12 | 49918501 | G | A | SPATS2 | p.R383Q | 0 | 70 | 0.119048 | 42 | WCM192_X1_1 | Tissue |
| 12 | 50727811 | C | T | FAM186A | p.M2193I | 0 | 84 | 0.133333 | 45 | WCM192_X1_1 | Tissue |
| 12 | 70351684 | C | A | MYRFL | p.L845M | 0 | 41 | 0.118644 | 59 | WCM192_X1_1 | Tissue |
| 12 | 8043659 | C | T | SLC2A14 | p.R22Q | 0 | 50 | 0.117647 | 34 | WCM192_X1_1 | Tissue |
| 13 | 32910749 | T | A | BRCA2 | p.F753I | 0 | 56 | 0.4 | 50 | WCM192_X1_1 | Tissue |
| 14 | 20404046 | C | G | OR4K1 | p.S74C | 0 | 63 | 0.096774 | 62 | WCM192_X1_1 | Tissue |
| 14 | 21542698 | C | T | ARHGEF40 | p.T270M | 0 | 78 | 0.10989 | 91 | WCM192_X1_1 | Tissue |
| 14 | 24771514 | A | G | NOP9 | p.R343G | 0 | 57 | 0.1 | 50 | WCM192_X1_1 | Tissue |
| 1 | 47139103 | C | T | TEX38 | p.A199V | 0 | 174 | 0.072289 | 166 | WCM192_X1_1 | Tissue |
| 1 | 48241061 | G | A | TRABD2B | p.P377L | 0 | 32 | 0.131579 | 38 | WCM192_X1_1 | Tissue |
| 1 | 49511311 | C | T | AGBL4 | p.S180N | 0 | 58 | 0.088235 | 34 | WCM192_X1_1 | Tissue |
| 14 | 99983489 | G | A | CCDC85C | p.S336L | 0 | 245 | 0.056338 | 213 | WCM192_X1_1 | Tissue |
| 15 | 45658378 | G | A | GATM | p.R282C | 0 | 37 | 0.354839 | 31 | WCM192_X1_1 | Tissue |
| 15 | 79750690 | G | A | KIAA1024 | p.R734H | 0 | 130 | 0.051546 | 97 | WCM192_X1_1 | Tissue |
| 1 | 5934559 | A | C | NPHP4 | p.F1068C | 0 | 92 | 0.0625 | 48 | WCM192_X1_1 | Tissue |
| 15 | 93749883 | G | A | AC112693.2 | p.R154Q | 0 | 338 | 0.060086 | 233 | WCM192_X1_1 | Tissue |
| 16 | 4625274 | A | G | C16orf96 | p.I265V | 0 | 176 | 0.069959 | 243 | WCM192_X1_1 | Tissue |
| 16 | 46771923 | T | C | MYLK3 | p.Q234R | 0 | 39 | 0.1875 | 32 | WCM192_X1_1 | Tissue |
| 16 | 58079165 | G | A | MMP15 | p.G609R | 0 | 133 | 0.213333 | 75 | WCM192_X1_1 | Tissue |
| 16 | 66792714 | T | C | CCDC79 | p.Q653R | 0 | 90 | 0.080645 | 124 | WCM192_X1_1 | Tissue |
| 16 | 75728368 | A | G | AC025287.1 | p.M150T | 0 | 162 | 0.078947 | 114 | WCM192_X1_1 | Tissue |
| 16 | 77246517 | C | A | SYCE1L | p.P210T | 0 | 42 | 0.142857 | 42 | WCM192_X1_1 | Tissue |
| 16 | 824442 | C | T | MSLNL | p.S249N | 0 | 112 | 0.394366 | 142 | WCM192_X1_1 | Tissue |
| 16 | 84497316 | A | C | ATP2C2 | p.Q940P | 0 | 110 | 0.065934 | 91 | WCM192_X1_1 | Tissue |
| 16 | 88501971 | T | A | ZNF469 | p.L2670Q | 0 | 139 | 0.119718 | 142 | WCM192_X1_1 | Tissue |
| 16 | 88803124 | T | C | PIEZO1 | p.R407G | 0 | 66 | 0.1875 | 48 | WCM192_X1_1 | Tissue |
| 17 | 11881792 | G | T | ZNF18 | p.H378N | 0 | 38 | 0.081633 | 49 | WCM192_X1_1 | Tissue |
| 17 | 17250197 | G | A | NT5M | p.R208H | 0 | 77 | 0.090909 | 66 | WCM192_X1_1 | Tissue |
| 17 | 19861701 | C | T | AKAP10 | p.R168Q | 0 | 81 | 0.394737 | 76 | WCM192_X1_1 | Tissue |
| 17 | 33805180 | G | C | SLFN12L | p.A373G | 0 | 157 | 0.086614 | 127 | WCM192_X1_1 | Tissue |
| 17 | 37319065 | C | T | ARL5C | p.V52M | 0 | 149 | 0.19917 | 241 | WCM192_X1_1 | Tissue |
| 17 | 37319103 | T | C | ARL5C | p.N39S | 0 | 81 | 0.195804 | 143 | WCM192_X1_1 | Tissue |
| 17 | 39186168 | A | C | KRTAP1-4 | p.S55A | 0 | 35 | 0.050847 | 59 | WCM192_X1_1 | Tissue |
| 17 | 42981499 | C | T | FAM187A | p.T101I | 0 | 115 | 0.07362 | 163 | WCM192_X1_1 | Tissue |
| 17 | 42982288 | A | G | FAM187A | p.H364R | 0 | 50 | 0.105263 | 38 | WCM192_X1_1 | Tissue |
| 17 | 71452153 | A | G | SDK2 | p.V124A | 0 | 104 | 0.097222 | 72 | WCM192_X1_1 | Tissue |
| 17 | 72368641 | G | A | GPR142 | p.D431N | 0 | 128 | 0.057325 | 157 | WCM192_X1_1 | Tissue |
| 17 | 73602081 | G | A | MYO15B | p.R207H | 0 | 83 | 0.060606 | 99 | WCM192_X1_1 | Tissue |
| 17 | 7577515 | T | G | TP53 | p.T256P | 0 | 114 | 0.447368 | 152 | WCM192_X1_1 | Tissue |
| 17 | 76490147 | C | T | DNAH17 | p.V2122I | 0 | 68 | 0.08547 | 117 | WCM192_X1_1 | Tissue |
| 18 | 21484014 | G | A | LAMA3 | p.A2146T | 0 | 111 | 0.057377 | 122 | WCM192_X1_1 | Tissue |
| 18 | 28659828 | G | A | DSC2 | p.L550F | 0 | 64 | 0.121212 | 66 | WCM192_X1_1 | Tissue |
| 18 | 51813694 | A | G | POLI | p.I371V | 0 | 73 | 0.065574 | 61 | WCM192_X1_1 | Tissue |
| 18 | 71740791 | C | G | FBXO15 | p.V480L | 0 | 81 | 0.075758 | 66 | WCM192_X1_1 | Tissue |
| 18 | 72021145 | T | A | C18orf63 | p.F548Y | 0 | 131 | 0.051813 | 193 | WCM192_X1_1 | Tissue |
| 19 | 1533972 | C | G | PLK5 | p.L253V | 0 | 90 | 0.097222 | 72 | WCM192_X1_1 | Tissue |
| 19 | 1535205 | G | C | PLK5 | p.G323R | 0 | 125 | 0.053191 | 94 | WCM192_X1_1 | Tissue |
| 19 | 16860493 | A | T | NWD1 | p.K347M | 0 | 179 | 0.064885 | 262 | WCM192_X1_1 | Tissue |
| 1 | 91840949 | C | A | HFM1 | p.A551S | 0 | 60 | 0.085714 | 35 | WCM192_X1_1 | Tissue |
| 19 | 18780018 | C | T | KLHL26 | p.A604V | 0 | 54 | 0.073684 | 95 | WCM192_X1_1 | Tissue |
| 19 | 37488299 | A | G | ZNF568 | p.E505G | 0 | 136 | 0.08 | 150 | WCM192_X1_1 | Tissue |
| 19 | 37488472 | C | G | ZNF568 | p.P563A | 0 | 101 | 0.053191 | 94 | WCM192_X1_1 | Tissue |
| 19 | 37488481 | G | C | ZNF568 | p.G566R | 0 | 102 | 0.052632 | 95 | WCM192_X1_1 | Tissue |
| 19 | 38160916 | C | T | ZNF781 | p.R45H | 0 | 34 | 0.512821 | 39 | WCM192_X1_1 | Tissue |
| 19 | 38377773 | G | A | WDR87 | p.R2141W | 0 | 95 | 0.061069 | 131 | WCM192_X1_1 | Tissue |
| 19 | 38997475 | G | A | RYR1 | p.G2900E | 0 | 195 | 0.114478 | 297 | WCM192_X1_1 | Tissue |
| 19 | 6040238 | G | T | RFX2 | p.T92N | 0 | 83 | 0.126316 | 95 | WCM192_X1_1 | Tissue |
| 20 | 18296291 | G | A | ZNF133 | p.G266R | 0 | 69 | 0.066667 | 60 | WCM192_X1_1 | Tissue |
| 20 | 31196431 | T | C | RP11- | p.L117P | 0 | 94 | 0.055556 | 108 | WCM192_X1_1 | Tissue |
| | | | | 410N8.4 | | | | | | | |
| 20 | 33345053 | C | T | NCOA6 | p.G500R | 0 | 107 | 0.075949 | 79 | WCM192_X1_1 | Tissue |
| 2 | 163124030 | C | T | IFIH1 | p.D953N | 0 | 45 | 0.146341 | 41 | WCM192_X1_1 | Tissue |
| 2 | 179914640 | G | A | CCDC141 | p.A10V | 0 | 221 | 0.070352 | 199 | WCM192_X1_1 | Tissue |
| 2 | 190660510 | G | A | PMS1 | p.D50N | 0 | 50 | 0.348485 | 66 | WCM192_X1_1 | Tissue |
| 22 | 18835298 | T | C | AC008132.13 | p.V285A | 0 | 50 | 0.050633 | 79 | WCM192_X1_1 | Tissue |
| 2 | 228566904 | T | G | SLC19A3 | p.K44T | 0 | 56 | 0.105263 | 38 | WCM192_X1_1 | Tissue |
| 22 | 31521404 | G | A | INPP5J | p.A227T | 0 | 104 | 0.100559 | 179 | WCM192_X1_1 | Tissue |
| 2 | 239133980 | C | T | AC016757.3 | p.G99R | 0 | 72 | 0.290323 | 62 | WCM192_X1_1 | Tissue |
| 22 | 41631282 | T | C | CHADL | p.Q710R | 0 | 196 | 0.055249 | 181 | WCM192_X1_1 | Tissue |
| 2 | 27930837 | C | T | AC074091.13 | p.G66R | 0 | 150 | 0.19469 | 113 | WCM192_X1_1 | Tissue |
| 2 | 54197829 | C | A | PSME4 | p.E31D | 0 | 54 | 0.076923 | 91 | WCM192_X1_1 | Tissue |
| 2 | 69046366 | A | T | ARHGAP25 | p.K371M | 0 | 203 | 0.074324 | 148 | WCM192_X1_1 | Tissue |
| 2 | 97633267 | G | C | FAM178B | p.P390A | 0 | 48 | 0.069444 | 72 | WCM192_X1_1 | Tissue |
| 3 | 113010544 | C | T | WDR52 | p.V1809I | 0 | 109 | 0.088889 | 90 | WCM192_X1_1 | Tissue |
| 3 | 113045325 | G | A | WDR52 | p.R1495W | 0 | 142 | 0.123077 | 65 | WCM192_X1_1 | Tissue |
| 3 | 113049270 | T | A | WDR52 | p.K1287N | 0 | 79 | 0.206897 | 87 | WCM192_X1_1 | Tissue |
| 3 | 126071216 | G | C | KLF15 | p.P184A | 0 | 159 | 0.073394 | 109 | WCM192_X1_1 | Tissue |
| 3 | 130113771 | G | A | COL6A5 | p.D1011N | 0 | 80 | 0.083333 | 96 | WCM192_X1_1 | Tissue |
| 3 | 138739355 | C | T | PRR23B | p.G50E | 0 | 55 | 0.117647 | 85 | WCM192_X1_1 | Tissue |
| 3 | 142840685 | C | T | CHST2 | p.R343C | 0 | 38 | 0.5 | 74 | WCM192_X1_1 | Tissue |
| 3 | 196311106 | G | T | FBXO45 | p.V260F | 0 | 100 | 0.326923 | 52 | WCM192_X1_1 | Tissue |
| 3 | 20042834 | C | T | PP2D1 | p.A260T | 0 | 123 | 0.065217 | 46 | WCM192_X1_1 | Tissue |
| 3 | 42700517 | G | C | ZBTB47 | p.G224R | 0 | 151 | 0.112782 | 133 | WCM192_X1_1 | Tissue |
| 3 | 42700518 | G | T | ZBTB47 | p.G224V | 0 | 151 | 0.112782 | 133 | WCM192_X1_1 | Tissue |
| 3 | 53126396 | C | A | RFT1 | p.A483S | 0 | 94 | 0.069652 | 201 | WCM192_X1_1 | Tissue |
| 3 | 56716922 | T | G | FAM208A | p.Q38P | 0 | 60 | 0.114286 | 70 | WCM192_X1_1 | Tissue |
| 3 | 9896259 | T | A | TTLL3 | p.M312K | 0 | 74 | 0.080645 | 62 | WCM192_X1_1 | Tissue |
| 4 | 114277956 | G | A | ANK2 | p.E2728K | 0 | 57 | 0.857143 | 35 | WCM192_X1_1 | Tissue |
| 4 | 122681487 | T | C | TMEM155 | p.T119A | 0 | 192 | 0.082707 | 133 | WCM192_X1_1 | Tissue |
| 4 | 13606303 | C | A | BOD1L1 | p.V741L | 0 | 99 | 0.050955 | 157 | WCM192_X1_1 | Tissue |
| 4 | 146567227 | G | A | MMAA | p.G218R | 0 | 95 | 0.092105 | 76 | WCM192_X1_1 | Tissue |
| 4 | 155410650 | T | C | DCHS2 | p.T620A | 0 | 83 | 0.138889 | 72 | WCM192_X1_1 | Tissue |
| 4 | 155410667 | G | A | DCHS2 | p.A614V | 0 | 82 | 0.140845 | 71 | WCM192_X1_1 | Tissue |
| 4 | 24801834 | C | G | SOD3 | p.R231G | 0 | 60 | 0.189189 | 74 | WCM192_X1_1 | Tissue |
| 4 | 25915957 | G | A | SMIM20 | p.A8T | 0 | 70 | 0.078652 | 89 | WCM192_X1_1 | Tissue |
| 4 | 36310065 | G | A | DTHD1 | p.R557Q | 0 | 92 | 0.055556 | 126 | WCM192_X1_1 | Tissue |
| 4 | 52938246 | G | A | SPATA18 | p.D228N | 0 | 311 | 0.411594 | 345 | WCM192_X1_1 | Tissue |
| 4 | 74019788 | T | A | ANKRD17 | p.D348V | 0 | 84 | 0.067416 | 89 | WCM192_X1_1 | Tissue |
| 5 | 134724624 | C | T | H2AFY | p.E54K | 0 | 112 | 0.142373 | 295 | WCM192_X1_1 | Tissue |
| 5 | 137765595 | G | A | KDM3B | p.A1612T | 0 | 140 | 0.604651 | 172 | WCM192_X1_1 | Tissue |
| 5 | 149406271 | C | T | HMGXB3 | p.A488V | 0 | 123 | 0.10084 | 119 | WCM192_X1_1 | Tissue |
| 5 | 151775064 | C | G | NMUR2 | p.S298T | 0 | 180 | 0.13308 | 263 | WCM192_X1_1 | Tissue |
| 5 | 167645814 | C | T | TENM2 | p.R1640W | 0 | 120 | 0.094737 | 95 | WCM192_X1_1 | Tissue |
| 5 | 180278091 | T | C | ZFP62 | p.N135S | 0 | 117 | 0.057971 | 69 | WCM192_X1_1 | Tissue |
| 5 | 6746450 | G | A | PAPD7 | p.G207S | 0 | 113 | 0.414062 | 128 | WCM192_X1_1 | Tissue |
| 5 | 93987532 | C | A | ANKRD32 | p.S288R | 0 | 81 | 0.196078 | 51 | WCM192_X1_1 | Tissue |
| 6 | 101215163 | A | G | ASCC3 | p.F485S | 0 | 108 | 0.058824 | 85 | WCM192_X1_1 | Tissue |
| 6 | 131148737 | G | A | SMLR1 | p.V62M | 0 | 56 | 0.059701 | 67 | WCM192_X1_1 | Tissue |
| 6 | 18171684 | G | A | KDM1B | p.G170S | 0 | 56 | 0.073171 | 41 | WCM192_X1_1 | Tissue |
| 6 | 30995532 | T | C | MUC22 | p.M775T | 0 | 57 | 0.066667 | 45 | WCM192_X1_1 | Tissue |
| 6 | 31526225 | G | A | NFKBIL1 | p.G328E | 0 | 57 | 0.157143 | 70 | WCM192_X1_1 | Tissue |
| 6 | 49485266 | C | A | GLYATL3 | p.Q104K | 0 | 142 | 0.057554 | 139 | WCM192_X1_1 | Tissue |
| 6 | 656256 | C | T | HUS1B | p.R230Q | 0 | 119 | 0.064935 | 77 | WCM192_X1_1 | Tissue |
| 6 | 73843172 | C | T | KCNQ5 | p.R426C | 0 | 49 | 0.516129 | 31 | WCM192_X1_1 | Tissue |
| 7 | 102098253 | A | C | ALKBH4 | p.I166S | 0 | 57 | 0.055556 | 54 | WCM192_X1_1 | Tissue |
| 7 | 12376811 | A | C | VWDE | p.F1485C | 0 | 116 | 0.105263 | 38 | WCM192_X1_1 | Tissue |
| 7 | 12428803 | G | A | VWDE | p.S142F | 0 | 188 | 0.106061 | 132 | WCM192_X1_1 | Tissue |
| 7 | 127999645 | C | T | PRRT4 | p.R134Q | 0 | 82 | 0.077922 | 154 | WCM192_X1_1 | Tissue |
| 7 | 141759668 | T | A | MGAM | p.S1321T | 0 | 102 | 0.083333 | 120 | WCM192_X1_1 | Tissue |
| 7 | 141759669 | C | G | MGAM | p.S1321C | 0 | 102 | 0.083333 | 120 | WCM192_X1_1 | Tissue |
| 7 | 46729069 | A | G | AC011294.3 | p.C83R | 0 | 89 | 0.121212 | 66 | WCM192_X1_1 | Tissue |
| 8 | 12957852 | C | T | DLC1 | p.R665H | 0 | 132 | 0.051095 | 137 | WCM192_X1_1 | Tissue |
| 8 | 61768636 | C | T | CHD7 | p.P2347S | 0 | 142 | 0.053398 | 206 | WCM192_X1_1 | Tissue |
| 9 | 271638 | C | T | DOCK8 | p.A22V | 0 | 144 | 0.111111 | 108 | WCM192_X1_1 | Tissue |
| 9 | 33572347 | T | C | ANKRD18B | p.F1009L | 0 | 66 | 0.140625 | 64 | WCM192_X1_1 | Tissue |
| 9 | 94172733 | C | T | NFIL3 | p.R95Q | 0 | 130 | 0.306533 | 199 | WCM192_X1_1 | Tissue |
| X | 27766045 | A | G | DCAF8L2 | p.T345A | 0 | 96 | 0.081818 | 110 | WCM192_X1_1 | Tissue |
| 11 | 129245968 | G | T | BARX2 | p.G13V | 0 | 62 | 0.135802 | 81 | WCM192_Z1_1 | Tissue |
| 11 | 134856546 | A | T | AP003062.1 | p.N385I | 0 | 137 | 0.240741 | 108 | WCM192_Z1_1 | Tissue |
| 1 | 22816907 | G | T | ZBTB40 | p.E156* | 0 | 46 | 0.14 | 50 | WCM192_Z1_1 | Tissue |
| 13 | 32910749 | T | A | BRCA2 | p.F753I | 0 | 56 | 0.3125 | 48 | WCM192_Z1_1 | Tissue |
| 13 | 58298857 | G | A | PCDH17 | p.R970H | 0 | 52 | 0.071429 | 42 | WCM192_Z1_1 | Tissue |
| 14 | 75989058 | C | G | BATF | p.S11R | 0 | 39 | 0.095238 | 42 | WCM192_Z1_1 | Tissue |
| 16 | 11272248 | G | T | CLEC16A | p.V955F | 0 | 79 | 0.116505 | 103 | WCM192_Z1_1 | Tissue |
| 16 | 824442 | C | T | MSLNL | p.S249N | 0 | 112 | 0.185185 | 81 | WCM192_Z1_1 | Tissue |
| 17 | 19861701 | C | T | AKAP10 | p.R168Q | 0 | 81 | 0.272727 | 88 | WCM192_Z1_1 | Tissue |
| 17 | 7577515 | T | G | TP53 | p.T256P | 0 | 114 | 0.268116 | 138 | WCM192_Z1_1 | Tissue |
| 1 | 91404282 | C | T | ZNF644 | p.E877K | 0 | 60 | 0.111111 | 72 | WCM192_Z1_1 | Tissue |
| 19 | 38160916 | C | T | ZNF781 | p.R45H | 0 | 34 | 0.407407 | 54 | WCM192_Z1_1 | Tissue |
| 2 | 190660510 | G | A | PMS1 | p.D50N | 0 | 50 | 0.297297 | 37 | WCM192_Z1_1 | Tissue |
| 3 | 142840685 | C | T | CHST2 | p.R343C | 0 | 38 | 0.54717 | 53 | WCM192_Z1_1 | Tissue |
| 3 | 196311106 | G | T | FBXO45 | p.V260F | 0 | 100 | 0.180851 | 94 | WCM192_Z1_1 | Tissue |
| 4 | 114277956 | G | A | ANK2 | p.E2728K | 0 | 57 | 0.4 | 45 | WCM192_Z1_1 | Tissue |
| 4 | 52938246 | G | A | SPATA18 | p.D228N | 0 | 311 | 0.084444 | 225 | WCM192_Z1_1 | Tissue |
| 5 | 137765595 | G | A | KDM3B | p.A1612T | 0 | 140 | 0.294643 | 112 | WCM192_Z1_1 | Tissue |
| 6 | 73843172 | C | T | KCNQ5 | p.R426C | 0 | 49 | 0.25 | 52 | WCM192_Z1_1 | Tissue |
| 8 | 133083719 | T | C | HHLA1 | p.K446R | 0 | 103 | 0.068376 | 117 | WCM192_Z1_1 | Tissue |
| 9 | 94172733 | C | T | NFIL3 | p.R95Q | 0 | 130 | 0.27027 | 148 | WCM192_Z1_1 | Tissue |
| 12 | 105550563 | G | T | KIAA1033 | p.A940S | 0 | 34 | 0.059701 | 67 | WCM195 | Plasma |
| 4 | 151408923 | C | T | LRBA | p.R2182H | 0 | 62 | 0.052632 | 95 | WCM195 | Plasma |
| 10 | 60124595 | T | C | UBE2D1 | p.I88T | 0 | 158 | 0.985714 | 70 | WCM195_Z8_1 | Tissue |
| 11 | 67800419 | G | A | NDUFS8 | p.D47N | 0 | 113 | 0.724409 | 127 | WCM195_Z8_1 | Tissue |
| 11 | 75141328 | C | T | KLHL35 | p.R116Q | 0 | 78 | 0.688889 | 45 | WCM195_Z8_1 | Tissue |
| 1 | 185959521 | C | T | HMCN1 | p.P1108L | 0 | 214 | 0.210784 | 204 | WCM195_Z8_1 | Tissue |
| 12 | 6230355 | A | G | VWF | p.S69P | 0 | 238 | 0.263636 | 220 | WCM195_Z8_1 | Tissue |
| 1 | 29630427 | G | A | PTPRU | p.R856H | 0 | 92 | 0.287879 | 66 | WCM195_Z8_1 | Tissue |
| 13 | 92345711 | G | A | GPC5 | p.R199Q | 0 | 162 | 0.054645 | 183 | WCM195_Z8_1 | Tissue |
| 1 | 43895513 | C | T | SZT2 | p.Q1379* | 0 | 142 | 0.606299 | 127 | WCM195_Z8_1 | Tissue |
| 14 | 94173103 | C | A | UNC79 | p.N2587K | 0 | 103 | 0.268817 | 93 | WCM195_Z8_1 | Tissue |
| 16 | 70599128 | A | G | SF3B3 | p.N875S | 0 | 147 | 0.352 | 125 | WCM195_Z8_1 | Tissue |
| 17 | 38346743 | G | A | RAPGEFL1 | p.S240N | 0 | 233 | 0.212698 | 315 | WCM195_Z8_1 | Tissue |
| 17 | 40695867 | C | T | NAGLU | p.R615C | 0 | 70 | 0.070588 | 85 | WCM195_Z8_1 | Tissue |
| 1 | 75078464 | G | A | C1orf173 | p.H344Y | 0 | 132 | 0.168224 | 214 | WCM195_Z8_1 | Tissue |
| 17 | 78013859 | C | A | CCDC40 | p.Y114* | 0 | 202 | 0.063158 | 190 | WCM195_Z8_1 | Tissue |
| 1 | 7812577 | C | T | CAMTA1 | p.R1648C | 0 | 77 | 0.646341 | 82 | WCM195_Z8_1 | Tissue |
| 18 | 29039945 | C | T | DSG3 | p.R219C | 0 | 91 | 0.528736 | 87 | WCM195_Z8_1 | Tissue |
| 18 | 33706813 | C | G | SLC39A6 | p.R53P | 0 | 175 | 0.444444 | 135 | WCM195_Z8_1 | Tissue |
| 19 | 10080580 | C | T | COL5A3 | p.G1319S | 0 | 192 | 0.108844 | 147 | WCM195_Z8_1 | Tissue |
| 19 | 57931006 | G | T | ZNF17 | p.C49F | 0 | 54 | 0.071429 | 70 | WCM195_Z8_1 | Tissue |
| 20 | 17437080 | G | A | PCSK2 | p.A397T | 0 | 52 | 0.454545 | 44 | WCM195_Z8_1 | Tissue |
| 20 | 17437081 | C | T | PCSK2 | p.A397V | 0 | 52 | 0.454545 | 44 | WCM195_Z8_1 | Tissue |
| 2 | 20454691 | C | T | PUM2 | p.D937N | 0 | 74 | 0.3875 | 80 | WCM195_Z8_1 | Tissue |
| 2 | 234197 | C | T | SH3YL1 | p.G123R | 0 | 276 | 0.521951 | 205 | WCM195_Z8_1 | Tissue |
| 22 | 38042916 | C | G | SH3BP1 | p.S339C | 0 | 80 | 0.677966 | 59 | WCM195_Z8_1 | Tissue |
| 3 | 113377900 | G | T | KIAA2018 | p.P877T | 0 | 104 | 0.418182 | 110 | WCM195_Z8_1 | Tissue |
| 3 | 38081110 | G | A | DLEC1 | p.V132M | 0 | 73 | 0.235849 | 106 | WCM195_Z8_1 | Tissue |
| 4 | 151408923 | C | T | LRBA | p.R2182H | 0 | 200 | 0.583333 | 168 | WCM195_Z8_1 | Tissue |
| 4 | 154709944 | G | A | SFRP2 | p.S15L | 0 | 341 | 0.304833 | 269 | WCM195_Z8_1 | Tissue |
| 4 | 47578826 | T | G | ATP10D | p.C1135G | 0 | 67 | 0.454545 | 77 | WCM195_Z8_1 | Tissue |
| 5 | 140734993 | G | A | PCDHGA4 | p.A76T | 0 | 190 | 0.506494 | 231 | WCM195_Z8_1 | Tissue |
| 5 | 150029332 | T | A | SYNPO | p.S743T | 0 | 212 | 0.45 | 160 | WCM195_Z8_1 | Tissue |
| 5 | 43533872 | G | T | PAIP1 | p.S407Y | 0 | 90 | 0.206349 | 126 | WCM195_Z8_1 | Tissue |
| 6 | 75848587 | G | A | COL12A1 | p.S1683F | 0 | 139 | 0.069565 | 115 | WCM195_Z8_1 | Tissue |
| 6 | 93956516 | C | A | EPHA7 | p.C907F | 0 | 120 | 0.330189 | 106 | WCM195_Z8_1 | Tissue |
| 7 | 151049965 | C | G | NUB1 | p.L138V | 0 | 71 | 0.542857 | 70 | WCM195_Z8_1 | Tissue |
| 9 | 136260749 | G | A | C9orf96 | p.R242Q | 0 | 79 | 0.354839 | 124 | WCM195_Z8_1 | Tissue |
| 10 | 8056602 | C | A | TAF3 | p.P893H | 0 | 65 | 0.197531 | 81 | WCM198 | Plasma |
| 1 | 103544263 | C | A | COL11A1 | p.A147S | 0 | 92 | 0.102564 | 78 | WCM198 | Plasma |
| 11 | 117985555 | C | A | TMPRSS4 | p.P308T | 0 | 41 | 0.210526 | 38 | WCM198 | Plasma |
| 11 | 124189793 | G | T | OR8D2 | p.L101I | 0 | 63 | 0.210526 | 57 | WCM198 | Plasma |
| 11 | 128839629 | G | A | ARHGAP32 | p.R1813C | 0 | 46 | 0.365385 | 52 | WCM198 | Plasma |
| 11 | 35211528 | T | G | CD44 | p.F195V | 0 | 50 | 0.283582 | 67 | WCM198 | Plasma |
| 1 | 165218668 | A | G | LMX1A | p.V158A | 0 | 37 | 0.302326 | 43 | WCM198 | Plasma |
| 11 | 984696 | C | G | AP2A2 | p.P253A | 0 | 45 | 0.28 | 50 | WCM198 | Plasma |
| 1 | 21191626 | G | C | EIF4G3 | p.L862V | 0 | 67 | 0.109589 | 73 | WCM198 | Plasma |
| 1 | 223983975 | G | T | TP53BP2 | p.Q756K | 0 | 59 | 0.054795 | 73 | WCM198 | Plasma |
| 1 | 231124091 | A | G | ARV1 | p.K67R | 0 | 62 | 0.2625 | 80 | WCM198 | Plasma |
| 12 | 53165684 | G | A | KRT76 | p.R412W | 0 | 74 | 0.255814 | 86 | WCM198 | Plasma |
| 12 | 54396308 | T | A | HOXC9 | p.F211L | 0 | 64 | 0.188406 | 69 | WCM198 | Plasma |
| 12 | 57038648 | A | C | ATP5B | p.I134M | 0 | 68 | 0.352941 | 68 | WCM198 | Plasma |
| 1 | 43166462 | C | T | YBX1 | p.R251C | 0 | 67 | 0.15873 | 63 | WCM198 | Plasma |
| 16 | 21982885 | T | G | UQCRC2 | p.F237C | 0 | 84 | 0.230769 | 78 | WCM198 | Plasma |
| 16 | 21982886 | T | A | UQCRC2 | p.F237L | 0 | 84 | 0.226667 | 75 | WCM198 | Plasma |
| 17 | 35946569 | T | C | SYNRG | p.Q110R | 0 | 71 | 0.212121 | 66 | WCM198 | Plasma |
| 17 | 41861224 | G | T | C17orf105 | p.R153I | 0 | 67 | 0.258824 | 85 | WCM198 | Plasma |
| 17 | 41861225 | A | T | C17orf105 | p.R153S | 0 | 66 | 0.238095 | 84 | WCM198 | Plasma |
| 17 | 49726562 | G | C | CA10 | p.I205M | 0 | 60 | 0.060976 | 82 | WCM198 | Plasma |
| 19 | 58024413 | G | A | ZNF773 | p.A111T | 0 | 62 | 0.25 | 76 | WCM198 | Plasma |
| 20 | 33734650 | C | T | EDEM2 | p.D66N | 0 | 45 | 0.294118 | 68 | WCM198 | Plasma |
| 20 | 61444348 | G | T | OGFR | p.V461L | 0 | 46 | 0.436364 | 55 | WCM198 | Plasma |
| 2 | 155157983 | G | A | GALNT13 | p.R346Q | 0 | 63 | 0.22449 | 49 | WCM198 | Plasma |
| 2 | 166870330 | G | A | SCN1A | p.T1210M | 0 | 59 | 0.253731 | 67 | WCM198 | Plasma |
| 2 | 179592875 | G | T | TTN | p.A6242E | 0 | 49 | 0.076923 | 39 | WCM198 | Plasma |
| 3 | 44763445 | A | G | ZNF502 | p.Y379C | 0 | 56 | 0.290323 | 62 | WCM198 | Plasma |
| 4 | 100131657 | C | T | ADH6 | p.D89N | 0 | 103 | 0.242718 | 103 | WCM198 | Plasma |
| 4 | 153247289 | G | A | FBXW7 | p.R505C | 0 | 54 | 0.20339 | 59 | WCM198 | Plasma |
| 4 | 76720821 | C | T | USO1 | p.T509I | 0 | 84 | 0.2625 | 80 | WCM198 | Plasma |
| 5 | 140720039 | T | A | PCDHGA2 | p.L501I | 0 | 67 | 0.277778 | 54 | WCM198 | Plasma |
| 5 | 157233022 | G | A | CLINT1 | p.T265I | 0 | 105 | 0.383838 | 99 | WCM198 | Plasma |
| 5 | 23521190 | C | A | PRDM9 | p.A137E | 0 | 61 | 0.15 | 100 | WCM198 | Plasma |
| 6 | 118606422 | G | C | SLC35F1 | p.S308T | 0 | 40 | 0.216667 | 60 | WCM198 | Plasma |
| 6 | 146480672 | C | T | GRM1 | p.R297* | 0 | 48 | 0.37931 | 58 | WCM198 | Plasma |
| 7 | 48033962 | C | A | SUN3 | p.V271L | 0 | 73 | 0.255814 | 86 | WCM198 | Plasma |
| 8 | 68931857 | A | C | PREX2 | p.H96P | 0 | 76 | 0.181818 | 99 | WCM198 | Plasma |
| 9 | 101258697 | T | G | GABBR2 | p.K244Q | 0 | 41 | 0.346154 | 78 | WCM198 | Plasma |
| 10 | 8056602 | C | A | TAF3 | p.P893H | 0 | 135 | 0.508621 | 116 | WCM198_Z1_1 | Tissue |
| 11 | 117985555 | C | A | TMPRSS4 | p.P308T | 0 | 79 | 0.413333 | 75 | WCM198_Z1_1 | Tissue |
| 11 | 124189793 | G | T | OR8D2 | p.L101I | 0 | 45 | 0.371429 | 35 | WCM198_Z1_1 | Tissue |
| 11 | 128839629 | G | A | ARHGAP32 | p.R1813C | 0 | 126 | 0.509202 | 163 | WCM198_Z1_1 | Tissue |
| 11 | 35211528 | T | G | CD44 | p.F195V | 0 | 72 | 0.44898 | 49 | WCM198_Z1_1 | Tissue |
| 1 | 15789913 | C | A | CELA2A | p.P130H | 0 | 36 | 0.078947 | 38 | WCM198_Z1_1 | Tissue |
| 1 | 165218668 | A | G | LMX1A | p.V158A | 0 | 86 | 0.487805 | 82 | WCM198_Z1_1 | Tissue |
| 1 | 179520434 | C | A | NPHS2 | p.L342F | 0 | 112 | 0.441379 | 145 | WCM198_Z1_1 | Tissue |
| 11 | 984696 | C | G | AP2A2 | p.P253A | 0 | 73 | 0.452055 | 73 | WCM198_Z1_1 | Tissue |
| 1 | 2252995 | C | T | MORN1 | p.D441N | 0 | 54 | 0.458333 | 48 | WCM198_Z1_1 | Tissue |
| 1 | 231124091 | A | G | ARV1 | p.K67R | 0 | 76 | 0.578125 | 64 | WCM198_Z1_1 | Tissue |
| 12 | 53165684 | G | A | KRT76 | p.R412W | 0 | 254 | 0.469194 | 211 | WCM198_Z1_1 | Tissue |
| 12 | 54396308 | T | A | HOXC9 | p.F211L | 0 | 76 | 0.66 | 50 | WCM198_Z1_1 | Tissue |
| 12 | 57038648 | A | C | ATP5B | p.I134M | 0 | 207 | 0.39521 | 167 | WCM198_Z1_1 | Tissue |
| 1 | 43166462 | C | T | YBX1 | p.R251C | 0 | 74 | 0.432836 | 67 | WCM198_Z1_1 | Tissue |
| 15 | 93545479 | C | A | CHD2 | p.Q1404K | 0 | 41 | 0.066667 | 45 | WCM198_Z1_1 | Tissue |
| 16 | 2812022 | G | T | SRRM2 | p.R498L | 0 | 50 | 0.285714 | 42 | WCM198_Z1_1 | Tissue |
| 17 | 35946569 | T | C | SYNRG | p.Q110R | 0 | 164 | 0.292929 | 198 | WCM198_Z1_1 | Tissue |
| 17 | 39503402 | G | C | KRT33A | p.L221V | 0 | 108 | 0.281818 | 110 | WCM198_Z1_1 | Tissue |
| 17 | 7573982 | C | A | TP53 | p.E349* | 0 | 98 | 0.909091 | 99 | WCM198_Z1_1 | Tissue |
| 19 | 15575069 | G | A | RASAL3 | p.A34V | 0 | 50 | 0.647059 | 68 | WCM198_Z1_1 | Tissue |
| 19 | 58024413 | G | A | ZNF773 | p.A111T | 0 | 58 | 0.409091 | 66 | WCM198_Z1_1 | Tissue |
| 20 | 33734650 | C | T | EDEM2 | p.D66N | 0 | 180 | 0.392405 | 158 | WCM198_Z1_1 | Tissue |
| 20 | 61444348 | G | T | OGFR | p.V461L | 0 | 118 | 0.634146 | 164 | WCM198_Z1_1 | Tissue |
| 2 | 219557366 | C | T | STK36 | p.S659F | 0 | 35 | 0.438596 | 57 | WCM198_Z1_1 | Tissue |
| 3 | 44763445 | A | G | ZNF502 | p.Y379C | 0 | 60 | 0.591837 | 49 | WCM198_Z1_1 | Tissue |
| 4 | 100131657 | C | T | ADH6 | p.D89N | 0 | 96 | 0.462963 | 108 | WCM198_Z1_1 | Tissue |
| 4 | 153247289 | G | A | FBXW7 | p.R505C | 0 | 80 | 0.468354 | 79 | WCM198_Z1_1 | Tissue |
| 4 | 76720821 | C | T | USO1 | p.T509I | 0 | 133 | 0.367188 | 128 | WCM198_Z1_1 | Tissue |
| 5 | 140720039 | T | A | PCDHGA2 | p.L501I | 0 | 91 | 0.33913 | 115 | WCM198_Z1_1 | Tissue |
| 5 | 157233022 | G | A | CLINT1 | p.T265I | 0 | 73 | 0.584416 | 77 | WCM198_Z1_1 | Tissue |
| 5 | 23521190 | C | A | PRDM9 | p.A137E | 0 | 74 | 0.188406 | 138 | WCM198_Z1_1 | Tissue |
| 6 | 34004374 | G | A | GRM4 | p.R505W | 0 | 71 | 0.275 | 80 | WCM198_Z1_1 | Tissue |
| 7 | 48033962 | C | A | SUN3 | p.V271L | 0 | 72 | 0.439394 | 66 | WCM198_Z1_1 | Tissue |
| 9 | 139815643 | C | T | TRAF2 | p.R372C | 0 | 50 | 0.1125 | 80 | WCM198_Z1_1 | Tissue |
| 11 | 1093105 | C | G | MUC2 | p.P1642A | 0 | 31 | 0.092308 | 65 | WCM199 | Plasma |
| 15 | 43748117 | A | C | TP53BP1 | p.S892A | 0 | 37 | 0.055556 | 72 | WCM199 | Plasma |
| 8 | 101276403 | A | T | RNF19A | p.L443I | 0 | 36 | 0.089286 | 56 | WCM199 | Plasma |
| 10 | 95791303 | G | C | PLCE1 | p.G167A | 0 | 102 | 0.5 | 68 | WCM199_Z3_1 | Tissue |
| 11 | 26558959 | G | A | ANO3 | p.R328H | 0 | 61 | 0.288136 | 59 | WCM199_Z3_1 | Tissue |
| 1 | 156821872 | C | G | INSRR | p.R250P | 0 | 44 | 0.634146 | 82 | WCM199_Z3_1 | Tissue |
| 1 | 44436817 | C | T | DPH2 | p.A147V | 0 | 242 | 0.381503 | 346 | WCM199_Z3_1 | Tissue |
| 15 | 40584342 | C | T | PLCB2 | p.A817T | 0 | 186 | 0.4 | 155 | WCM199_Z3_1 | Tissue |
| 19 | 33623148 | G | A | WDR88 | p.A25T | 0 | 82 | 0.3125 | 64 | WCM199_Z3_1 | Tissue |
| 1 | 94564442 | G | A | ABCA4 | p.R226C | 0 | 108 | 0.053763 | 93 | WCM199_Z3_1 | Tissue |
| 2 | 233323651 | G | A | ALPI | p.R461H | 0 | 81 | 0.085106 | 47 | WCM199_Z3_1 | Tissue |
| 3 | 185782211 | T | C | ETV5 | p.H322R | 0 | 86 | 0.324176 | 182 | WCM199_Z3_1 | Tissue |
| 4 | 46043213 | G | A | GABRG1 | p.P397L | 0 | 34 | 0.5 | 34 | WCM199_Z3_1 | Tissue |
| 6 | 44358075 | A | T | CDC5L | p.H39L | 0 | 146 | 0.423077 | 130 | WCM199_Z3_1 | Tissue |
| 8 | 24356786 | T | C | ADAM7 | p.V627A | 0 | 102 | 0.210191 | 157 | WCM199_Z3_1 | Tissue |
| 8 | 27764754 | C | G | SCARA5 | p.G336A | 0 | 88 | 0.062016 | 129 | WCM199_Z3_1 | Tissue |
| 9 | 2056721 | C | T | SMARCA2 | p.T408M | 0 | 127 | 0.366197 | 71 | WCM199_Z3_1 | Tissue |
| 9 | 35665136 | G | A | ARHGEF39 | p.P11S | 0 | 129 | 0.405063 | 79 | WCM199_Z3_1 | Tissue |
| 15 | 28412883 | C | A | HERC2 | p.D3502Y | 0 | 230 | 0.064516 | 217 | WCM1_T | Tissue |
| 17 | 7577538 | C | T | TP53 | p.R248Q | 0 | 100 | 0.054945 | 91 | WCM1_T | Tissue |
| 4 | 55152038 | G | A | PDGFRA | p.V824I | 0 | 491 | 0.053305 | 469 | WCM1_T | Tissue |
| 11 | 19206549 | C | A | CSRP3 | p.S153I | 0 | 36 | 0.060241 | 83 | WCM2 | Plasma |
| 11 | 6867517 | G | A | OR10A5 | p.V202I | 0 | 36 | 0.150685 | 73 | WCM2 | Plasma |
| 12 | 57598451 | A | C | LRP1 | p.N3705H | 0 | 32 | 0.056818 | 88 | WCM2 | Plasma |
| 13 | 45602098 | A | G | GPALPP1 | p.N328S | 0 | 54 | 0.07377 | 122 | WCM2 | Plasma |
| 14 | 47343266 | G | A | MDGA2 | p.R790C | 0 | 38 | 0.116505 | 103 | WCM2 | Plasma |
| 16 | 424079 | C | T | TMEM8A | p.D610N | 0 | 35 | 0.094118 | 85 | WCM2 | Plasma |
| 19 | 49926533 | G | C | PTH2 | p.L22V | 0 | 32 | 0.058824 | 85 | WCM2 | Plasma |
| 5 | 37121743 | C | A | C5orf42 | p.C3000F | 0 | 66 | 0.228814 | 118 | WCM2 | Plasma |
| 10 | 84118501 | C | T | NRG3 | p.T277M | 0 | 63 | 0.318182 | 44 | WCM201_Z1_1 | Tissue |
| 1 | 110032692 | A | G | ATXN7L2 | p.D393G | 0 | 66 | 0.284483 | 116 | WCM201_Z1_1 | Tissue |
| 11 | 123894494 | C | A | OR10G9 | p.L259M | 0 | 141 | 0.203488 | 172 | WCM201_Z1_1 | Tissue |
| 11 | 1477725 | G | A | BRSK2 | p.G606S | 0 | 141 | 0.165049 | 103 | WCM201_Z1_1 | Tissue |
| 1 | 153410724 | C | T | S100A7L2 | p.D39N | 0.004831 | 207 | 0.09375 | 192 | WCM201_Z1_1 | Tissue |
| 11 | 66982825 | C | G | KDM2A | p.D167E | 0 | 93 | 0.358974 | 78 | WCM201_Z1_1 | Tissue |
| 1 | 180885828 | C | T | KIAA1614 | p.H197Y | 0 | 194 | 0.129032 | 186 | WCM201_Z1_1 | Tissue |
| 1 | 186092324 | C | G | HMCN1 | p.S4157R | 0 | 89 | 0.068376 | 117 | WCM201_Z1_1 | Tissue |
| 1 | 201195129 | G | A | IGFN1 | p.R3555H | 0 | 55 | 0.152941 | 85 | WCM201_Z1_1 | Tissue |
| 12 | 32136327 | C | T | KIAA1551 | p.A813V | 0 | 59 | 0.084337 | 83 | WCM201_Z1_1 | Tissue |
| 1 | 248512142 | G | T | OR14C36 | p.Q22H | 0 | 106 | 0.07 | 100 | WCM201_Z1_1 | Tissue |
| 12 | 80943507 | G | A | PTPRQ | p.E1423K | 0 | 84 | 0.079245 | 265 | WCM201_Z1_1 | Tissue |
| 14 | 77750142 | G | A | POMT2 | p.R551W | 0 | 49 | 0.453333 | 75 | WCM201_Z1_1 | Tissue |
| 15 | 91434792 | C | G | FES | p.Y513* | 0.009615 | 104 | 0.122222 | 90 | WCM201_Z1_1 | Tissue |
| 16 | 19711736 | C | T | C16orf62 | p.Q944* | 0 | 199 | 0.364407 | 236 | WCM201_Z1_1 | Tissue |
| 17 | 27254014 | G | C | PHF12 | p.R106G | 0 | 105 | 0.268519 | 108 | WCM201_Z1_1 | Tissue |
| 17 | 29527473 | G | A | NF1 | p.A308T | 0 | 109 | 0.387755 | 147 | WCM201_Z1_1 | Tissue |
| 17 | 56355332 | G | A | MPO | p.R354C | 0 | 42 | 0.267857 | 56 | WCM201_Z1_1 | Tissue |
| 17 | 7577505 | T | A | TP53 | p.D259V | 0 | 103 | 0.60396 | 101 | WCM201_Z1_1 | Tissue |
| 17 | 76134755 | C | T | TMC8 | p.R589C | 0 | 140 | 0.098039 | 204 | WCM201_Z1_1 | Tissue |
| 18 | 76754198 | G | A | SALL3 | p.R736H | 0 | 60 | 0.266667 | 45 | WCM201_Z1_1 | Tissue |
| 19 | 46388174 | C | T | IRF2BP1 | p.A287T | 0 | 56 | 0.103448 | 58 | WCM201_Z1_1 | Tissue |
| 20 | 49626457 | C | T | KCNG1 | p.R140H | 0 | 61 | 0.061728 | 81 | WCM201_Z1_1 | Tissue |
| 2 | 186604169 | G | A | FSIP2 | p.V66I | 0 | 52 | 0.186441 | 59 | WCM201_Z1_1 | Tissue |
| 4 | 46053577 | G | A | GABRG1 | p.A332V | 0 | 57 | 0.362069 | 58 | WCM201_Z1_1 | Tissue |
| 5 | 153056685 | G | T | GRIA1 | p.W331C | 0 | 93 | 0.131868 | 91 | WCM201_Z1_1 | Tissue |
| 6 | 36334526 | G | T | ETV7 | p.P308T | 0 | 37 | 0.478261 | 46 | WCM201_Z1_1 | Tissue |
| 6 | 36981471 | G | A | FGD2 | p.R205Q | 0 | 117 | 0.073171 | 123 | WCM201_Z1_1 | Tissue |
| 7 | 137148323 | G | A | DGKI | p.R891C | 0 | 83 | 0.317308 | 104 | WCM201_Z1_1 | Tissue |
| 8 | 146033125 | G | A | ZNF517 | p.R275Q | 0 | 31 | 0.071429 | 42 | WCM201_Z1_1 | Tissue |
| 1 | 169572276 | G | T | SELP | p.P565T | 0 | 33 | 0.057143 | 70 | WCM207 | Plasma |
| 12 | 124303504 | A | G | DNAH10 | p.D1145G | 0 | 66 | 0.394737 | 76 | WCM207 | Plasma |
| 16 | 72822473 | G | T | ZFHX3 | p.D3234E | 0 | 37 | 0.447368 | 38 | WCM207 | Plasma |
| 17 | 3030034 | T | A | OR1G1 | p.D271V | 0 | 57 | 0.752577 | 97 | WCM207 | Plasma |
| 17 | 7579349 | A | C | TP53 | p.F113C | 0 | 55 | 0.621212 | 66 | WCM207 | Plasma |
| 18 | 67534634 | C | T | CD226 | p.E282K | 0 | 41 | 0.618182 | 55 | WCM207 | Plasma |
| 19 | 16279000 | C | A | CIB3 | p.M98I | 0 | 40 | 0.227273 | 66 | WCM207 | Plasma |
| 19 | 35505186 | C | T | GRAMD1A | p.P322S | 0 | 39 | 0.375 | 96 | WCM207 | Plasma |
| 2 | 183053727 | T | A | PDE1A | p.M412L | 0 | 61 | 0.52439 | 82 | WCM207 | Plasma |
| 2 | 202400781 | T | C | ALS2CR11 | p.Y490C | 0 | 43 | 0.383721 | 86 | WCM207 | Plasma |
| 2 | 232326373 | T | C | NCL | p.D164G | 0 | 38 | 0.191781 | 73 | WCM207 | Plasma |
| 2 | 232326380 | C | T | NCL | p.D162N | 0 | 39 | 0.180556 | 72 | WCM207 | Plasma |
| 5 | 80638502 | T | A | ACOT12 | p.T373S | 0 | 61 | 0.052632 | 114 | WCM207 | Plasma |
| 6 | 166845937 | C | G | RPS6KA2 | p.E458D | 0 | 46 | 0.211111 | 90 | WCM207 | Plasma |
| 6 | 166845945 | C | G | RPS6KA2 | p.E456Q | 0 | 45 | 0.197802 | 91 | WCM207 | Plasma |
| 6 | 56380269 | C | A | DST | p.R5865S | 0 | 66 | 0.436782 | 87 | WCM207 | Plasma |
| 7 | 121650401 | G | T | PTPRZ1 | p.G434V | 0 | 52 | 0.336957 | 92 | WCM207 | Plasma |
| 9 | 131389743 | G | A | SPTAN1 | p.A2214T | 0 | 57 | 0.396226 | 106 | WCM207 | Plasma |
| 9 | 139354245 | C | T | SEC16A | p.E1541K | 0 | 31 | 0.333333 | 48 | WCM207 | Plasma |
| 9 | 96857616 | G | A | PTPDC1 | p.G158R | 0 | 52 | 0.428571 | 91 | WCM207 | Plasma |
| 1 | 109395210 | A | T | AKNAD1 | p.I26N | 0.007692 | 130 | 0.676768 | 99 | WCM207_X1_1 | Tissue |
| 11 | 118211224 | G | A | CD3D | p.T47M | 0 | 125 | 0.479452 | 146 | WCM207_X1_1 | Tissue |
| 1 | 112329574 | C | T | KCND3 | p.A421T | 0.007463 | 134 | 0.333333 | 108 | WCM207_X1_1 | Tissue |
| 1 | 49005396 | G | A | AGBL4 | p.R428W | 0 | 120 | 0.755725 | 131 | WCM207_X1_1 | Tissue |
| 15 | 42115750 | C | T | MAPKBP1 | p.P1241L | 0 | 115 | 0.478261 | 46 | WCM207_X1_1 | Tissue |
| 17 | 19646602 | T | G | ALDH3A1 | p.T113P | 0 | 243 | 0.438095 | 210 | WCM207_X1_1 | Tissue |
| 17 | 3030034 | T | A | OR1G1 | p.D271V | 0.005495 | 182 | 0.988636 | 88 | WCM207_X1_1 | Tissue |
| 17 | 4646643 | G | A | ZMYND15 | p.R397Q | 0 | 64 | 0.075 | 40 | WCM207_X1_1 | Tissue |
| 17 | 7579349 | A | C | TP53 | p.F113C | 0 | 235 | 0.9 | 150 | WCM207_X1_1 | Tissue |
| 18 | 67534634 | C | T | CD226 | p.E282K | 0 | 101 | 0.982143 | 56 | WCM207_X1_1 | Tissue |
| 19 | 16279000 | C | A | CIB3 | p.M98I | 0 | 147 | 0.210227 | 176 | WCM207_X1_1 | Tissue |
| 19 | 35505186 | C | T | GRAMD1A | p.P322S | 0 | 58 | 0.538462 | 78 | WCM207_X1_1 | Tissue |
| 19 | 55785879 | A | G | HSPBP1 | p.V176A | 0 | 53 | 0.492754 | 69 | WCM207_X1_1 | Tissue |
| 2 | 183053727 | T | A | PDE1A | p.M412L | 0 | 96 | 0.584615 | 65 | WCM207_X1_1 | Tissue |
| 22 | 24040502 | A | T | RGL4 | p.Q455L | 0 | 73 | 0.54386 | 57 | WCM207_X1_1 | Tissue |
| 3 | 138724834 | C | T | PRR23A | p.D93N | 0 | 99 | 0.469388 | 98 | WCM207_X1_1 | Tissue |
| 6 | 166844093 | C | G | RPS6KA2 | p.D477H | 0 | 82 | 0.33871 | 62 | WCM207_X1_1 | Tissue |
| 6 | 56380269 | C | A | DST | p.R5865S | 0 | 125 | 0.596491 | 114 | WCM207_X1_1 | Tissue |
| 7 | 121650401 | G | T | PTPRZ1 | p.G434V | 0 | 72 | 0.507463 | 67 | WCM207_X1_1 | Tissue |
| 7 | 16605977 | G | A | LRRC72 | p.R156H | 0 | 83 | 0.096774 | 62 | WCM207_X1_1 | Tissue |
| 9 | 104238426 | G | A | TMEM246 | p.R317W | 0 | 129 | 0.48 | 150 | WCM207_X1_1 | Tissue |
| 9 | 131389743 | G | A | SPTAN1 | p.A2214T | 0 | 91 | 0.595041 | 121 | WCM207_X1_1 | Tissue |
| 9 | 138556089 | G | A | LCN9 | p.V60I | 0 | 101 | 0.509615 | 104 | WCM207_X1_1 | Tissue |
| 9 | 139354245 | C | T | SEC16A | p.E1541K | 0 | 108 | 0.52381 | 126 | WCM207_X1_1 | Tissue |
| X | 109695439 | G | T | RGAG1 | p.A532S | 0 | 44 | 0.073171 | 41 | WCM207_X1_1 | Tissue |
| 11 | 35211496 | G | A | CD44 | p.S184N | 0 | 41 | 0.375 | 72 | WCM212 | Plasma |
| 1 | 247615175 | A | G | OR2B11 | p.L37P | 0 | 42 | 0.168539 | 89 | WCM212 | Plasma |
| 14 | 23044016 | T | A | DAD1 | p.N110I | 0 | 42 | 0.055556 | 72 | WCM212 | Plasma |
| 16 | 67974112 | C | G | LCAT | p.G340R | 0 | 34 | 0.087719 | 57 | WCM212 | Plasma |
| 16 | 72141552 | A | G | DHX38 | p.T940A | 0 | 40 | 0.317073 | 82 | WCM212 | Plasma |
| 19 | 49003080 | G | A | LMTK3 | p.P416S | 0 | 42 | 0.070423 | 71 | WCM212 | Plasma |
| 2 | 191923448 | G | A | STAT4 | p.A361V | 0 | 120 | 0.05303 | 132 | WCM212 | Plasma |
| 2 | 197948168 | C | T | ANKRD44 | p.G436E | 0 | 34 | 0.058824 | 102 | WCM212 | Plasma |
| 2 | 44209481 | T | C | LRPPRC | p.N81S | 0 | 38 | 0.087379 | 103 | WCM212 | Plasma |
| 5 | 10236707 | T | G | FAM173B | p.K109N | 0 | 39 | 0.081967 | 122 | WCM212 | Plasma |
| 5 | 167855749 | G | A | WWC1 | p.E653K | 0 | 42 | 0.109091 | 110 | WCM212 | Plasma |
| 6 | 144811308 | A | T | UTRN | p.R1412S | 0 | 41 | 0.106383 | 94 | WCM212 | Plasma |
| 7 | 37916520 | C | T | NME8 | p.A302V | 0 | 54 | 0.440367 | 109 | WCM212 | Plasma |
| 7 | 48273713 | G | A | ABCA13 | p.E288K | 0 | 41 | 0.060976 | 82 | WCM212 | Plasma |
| 8 | 17731929 | C | T | FGL1 | p.D116N | 0 | 35 | 0.107143 | 56 | WCM212 | Plasma |
| 8 | 9473125 | G | A | TNKS | p.R311Q | 0 | 31 | 0.075472 | 53 | WCM212 | Plasma |
| 9 | 32467922 | G | A | DDX58 | p.L675F | 0 | 36 | 0.127273 | 110 | WCM212 | Plasma |
| 9 | 37744799 | G | C | FRMPD1 | p.E924Q | 0 | 31 | 0.054054 | 74 | WCM212 | Plasma |
| 10 | 13370389 | T | G | SEPHS1 | p.Q238P | 0 | 54 | 0.588235 | 34 | WCM212_Z2_1 | Tissue |
| 11 | 114401609 | A | G | NXPE1 | p.S41P | 0 | 70 | 0.236364 | 110 | WCM212_Z2_1 | Tissue |
| 11 | 123886634 | C | T | OR10G4 | p.S118F | 0 | 101 | 0.160839 | 143 | WCM212_Z2_1 | Tissue |
| 11 | 35211496 | G | A | CD44 | p.S184N | 0 | 48 | 0.357143 | 42 | WCM212_Z2_1 | Tissue |
| 1 | 209849198 | C | T | G0S2 | p.R57W | 0 | 44 | 0.555556 | 45 | WCM212_Z2_1 | Tissue |
| 1 | 247615175 | A | G | OR2B11 | p.L37P | 0 | 34 | 0.3125 | 32 | WCM212_Z2_1 | Tissue |
| 1 | 248186052 | C | G | OR2L5 | p.T268R | 0 | 83 | 0.685393 | 89 | WCM212_Z2_1 | Tissue |
| 1 | 40092289 | C | T | HEYL | p.V293M | 0 | 103 | 0.527273 | 55 | WCM212_Z2_1 | Tissue |
| 14 | 21813332 | A | G | RPGRIP1 | p.N1198S | 0 | 87 | 0.25 | 60 | WCM212_Z2_1 | Tissue |
| 16 | 27221835 | G | C | KDM8 | p.A131P | 0 | 58 | 0.338983 | 59 | WCM212_Z2_1 | Tissue |
| 16 | 72141552 | A | G | DHX38 | p.T940A | 0 | 146 | 0.414141 | 99 | WCM212_Z2_1 | Tissue |
| 16 | 737394 | G | A | WDR24 | p.R358C | 0 | 168 | 0.247059 | 170 | WCM212_Z2_1 | Tissue |
| 17 | 39681209 | G | T | KRT19 | p.D182E | 0 | 86 | 0.326733 | 101 | WCM212_Z2_1 | Tissue |
| 18 | 11066210 | G | A | PIEZO2 | p.R26* | 0 | 163 | 0.364964 | 137 | WCM212_Z2_1 | Tissue |
| 19 | 41800254 | G | A | HNRNPUL1 | p.M426I | 0 | 59 | 0.061538 | 65 | WCM212_Z2_1 | Tissue |
| 22 | 41621911 | T | A | L3MBTL2 | p.C490* | 0 | 179 | 0.052941 | 170 | WCM212_Z2_1 | Tissue |
| 2 | 73675876 | C | T | ALMS1 | p.T740I | 0 | 55 | 0.193548 | 31 | WCM212_Z2_1 | Tissue |
| 5 | 10236707 | T | G | FAM173B | p.K109N | 0 | 71 | 0.178082 | 73 | WCM212_Z2_1 | Tissue |
| 5 | 149001606 | C | A | ARHGEF37 | p.A439E | 0 | 137 | 0.272109 | 147 | WCM212_Z2_1 | Tissue |
| 5 | 175110957 | G | A | HRH2 | p.G241R | 0 | 132 | 0.392857 | 140 | WCM212_Z2_1 | Tissue |
| 7 | 37916520 | C | T | NME8 | p.A302V | 0 | 68 | 0.397727 | 88 | WCM212_Z2_1 | Tissue |
| 8 | 11710911 | C | T | CTSB | p.R18Q | 0 | 61 | 0.3125 | 32 | WCM212_Z2_1 | Tissue |
| 8 | 144944813 | G | A | EPPK1 | p.A870V | 0 | 57 | 0.270833 | 48 | WCM212_Z2_1 | Tissue |
| 8 | 145540305 | G | A | DGAT1 | p.S460L | 0 | 81 | 0.26087 | 115 | WCM212_Z2_1 | Tissue |
| 11 | 59577366 | G | A | MRPL16 | p.A28V | 0 | 42 | 0.069767 | 43 | WCM213 | Plasma |
| 17 | 76462868 | C | T | DNAH17 | p.V2932M | 0 | 35 | 0.058824 | 68 | WCM213 | Plasma |
| 18 | 5445164 | G | A | EPB41L3 | p.T154M | 0 | 42 | 0.061728 | 81 | WCM213 | Plasma |
| 2 | 208489076 | C | G | METTL21A | p.E8D | 0 | 43 | 0.059701 | 67 | WCM213 | Plasma |
| 2 | 55436588 | G | C | CLHC1 | p.P256A | 0 | 56 | 0.055556 | 90 | WCM213 | Plasma |
| 3 | 49720558 | A | G | APEH | p.K691R | 0 | 37 | 0.09589 | 73 | WCM213 | Plasma |
| 10 | 112541631 | T | C | RBM20 | p.F422L | 0 | 295 | 0.083682 | 239 | WCM213_X1_1 | Tissue |
| 1 | 109466823 | T | C | GPSM2 | p.L601P | 0 | 61 | 0.410256 | 39 | WCM213_X1_1 | Tissue |
| 11 | 10506394 | A | T | AMPD3 | p.E215V | 0 | 175 | 0.116438 | 146 | WCM213_X1_1 | Tissue |
| 11 | 124875035 | C | T | CCDC15 | p.L780F | 0 | 135 | 0.113402 | 97 | WCM213_X1_1 | Tissue |
| 11 | 1255834 | G | A | MUC5B | p.E852K | 0 | 173 | 0.088496 | 113 | WCM213_X1_1 | Tissue |
| 1 | 114429257 | C | A | BCL2L15 | p.A51S | 0 | 70 | 0.073171 | 41 | WCM213_X1_1 | Tissue |
| 11 | 17741910 | G | A | MYOD1 | p.S194N | 0 | 47 | 0.150943 | 53 | WCM213_X1_1 | Tissue |
| 11 | 36597306 | G | T | RAG1 | p.V818L | 0 | 145 | 0.107692 | 130 | WCM213_X1_1 | Tissue |
| 11 | 36614203 | G | A | RAG2 | p.R506C | 0 | 117 | 0.138889 | 108 | WCM213_X1_1 | Tissue |
| 11 | 71259729 | G | A | KRTAP5-9 | p.G9D | 0 | 164 | 0.081081 | 111 | WCM213_X1_1 | Tissue |
| 1 | 176992672 | C | G | ASTN1 | p.D436H | 0 | 183 | 0.087719 | 228 | WCM213_X1_1 | Tissue |
| 1 | 179965945 | C | G | CEP350 | p.S218C | 0 | 199 | 0.057692 | 208 | WCM213_X1_1 | Tissue |
| 1 | 180012277 | G | T | CEP350 | p.Q1483H | 0 | 188 | 0.066116 | 121 | WCM213_X1_1 | Tissue |
| 1 | 185143976 | A | T | SWT1 | p.S233C | 0 | 35 | 0.133333 | 60 | WCM213_X1_1 | Tissue |
| 11 | 92085484 | G | A | FAT3 | p.G69D | 0 | 49 | 0.282051 | 39 | WCM213_X1_1 | Tissue |
| 12 | 104179177 | G | C | NT5DC3 | p.T422R | 0 | 153 | 0.227273 | 110 | WCM213_X1_1 | Tissue |
| 1 | 231131571 | A | C | ARV1 | p.K172Q | 0 | 36 | 0.066667 | 60 | WCM213_X1_1 | Tissue |
| 12 | 7287982 | G | T | CLSTN3 | p.R148L | 0 | 215 | 0.12069 | 116 | WCM213_X1_1 | Tissue |
| 13 | 58207629 | C | A | PCDH17 | p.L317M | 0 | 133 | 0.085714 | 70 | WCM213_X1_1 | Tissue |
| 14 | 76123838 | C | T | C14orf1 | p.R3H | 0 | 216 | 0.061644 | 146 | WCM213_X1_1 | Tissue |
| 15 | 41146858 | T | C | SPINT1 | p.L379P | 0 | 126 | 0.070707 | 99 | WCM213_X1_1 | Tissue |
| 15 | 43695979 | G | C | TUBGCP4 | p.Q611H | 0 | 69 | 0.125 | 72 | WCM213_X1_1 | Tissue |
| 15 | 50555481 | T | G | HDC | p.D52A | 0 | 197 | 0.089552 | 67 | WCM213_X1_1 | Tissue |
| 16 | 88691615 | C | T | ZC3H18 | p.T683M | 0 | 53 | 0.0625 | 48 | WCM213_X1_1 | Tissue |
| 17 | 1840679 | C | T | RTN4RL1 | p.G146D | 0 | 58 | 0.087719 | 57 | WCM213_X1_1 | Tissue |
| 17 | 2996057 | G | C | OR1D2 | p.I78M | 0 | 104 | 0.196721 | 122 | WCM213_X1_1 | Tissue |
| 17 | 60767610 | C | G | MRC2 | p.S1279C | 0 | 204 | 0.055814 | 215 | WCM213_X1_1 | Tissue |
| 17 | 79425974 | G | A | RP11- | p.A1862T | 0 | 32 | 0.061224 | 49 | WCM213_X1_1 | Tissue |
| | | | | 1055B8.7 | | | | | | | |
| 17 | 79912150 | T | C | NOTUM | p.H389R | 0 | 44 | 0.107692 | 65 | WCM213_X1_1 | Tissue |
| 19 | 10129542 | G | A | RDH8 | p.R133Q | 0 | 81 | 0.120482 | 83 | WCM213_X1_1 | Tissue |
| 19 | 14938182 | C | G | OR7A5 | p.S291T | 0 | 95 | 0.180556 | 72 | WCM213_X1_1 | Tissue |
| 19 | 15769192 | C | T | CYP4F3 | p.R412W | 0 | 111 | 0.097826 | 92 | WCM213_X1_1 | Tissue |
| 19 | 46443275 | A | T | NOVA2 | p.I442N | 0 | 115 | 0.086957 | 69 | WCM213_X1_1 | Tissue |
| 19 | 55687474 | C | G | SYT5 | p.E91Q | 0 | 123 | 0.084034 | 119 | WCM213_X1_1 | Tissue |
| 20 | 34091548 | A | G | CEP250 | p.Q1784R | 0 | 181 | 0.051813 | 193 | WCM213_X1_1 | Tissue |
| 20 | 56078638 | G | A | CTCFL | p.S565L | 0 | 153 | 0.05814 | 86 | WCM213_X1_1 | Tissue |
| 20 | 61950905 | A | T | COL20A1 | p.D958V | 0 | 88 | 0.066176 | 136 | WCM213_X1_1 | Tissue |
| 21 | 16337311 | A | C | NRIP1 | p.L1068R | 0 | 75 | 0.061538 | 65 | WCM213_X1_1 | Tissue |
| 21 | 45950952 | G | A | TSPEAR | p.R203W | 0 | 122 | 0.066667 | 120 | WCM213_X1_1 | Tissue |
| 21 | 47821593 | G | C | PCNT | p.Q1640H | 0 | 245 | 0.107692 | 260 | WCM213_X1_1 | Tissue |
| 2 | 208489076 | C | G | METTL21A | p.E8D | 0 | 75 | 0.296296 | 81 | WCM213_X1_1 | Tissue |
| 2 | 211541747 | A | G | CPS1 | p.S1431G | 0 | 49 | 0.105263 | 38 | WCM213_X1_1 | Tissue |
| 22 | 24581790 | A | G | SUSD2 | p.Y411C | 0 | 140 | 0.108434 | 83 | WCM213_X1_1 | Tissue |
| 22 | 30074280 | G | T | NF2 | p.M514I | 0 | 91 | 0.056604 | 53 | WCM213_X1_1 | Tissue |
| 2 | 55436588 | G | C | CLHC1 | p.P256A | 0 | 73 | 0.102564 | 78 | WCM213_X1_1 | Tissue |
| 2 | 55750956 | C | A | CCDC104 | p.Q94K | 0 | 95 | 0.103448 | 58 | WCM213_X1_1 | Tissue |
| 2 | 70164394 | G | A | MXD1 | p.V116I | 0 | 52 | 0.096774 | 62 | WCM213_X1_1 | Tissue |
| 3 | 124103725 | G | A | KALRN | p.A600T | 0 | 88 | 0.066116 | 121 | WCM213_X1_1 | Tissue |
| 3 | 127842441 | C | T | RUVBL1 | p.E43K | 0 | 67 | 0.098901 | 91 | WCM213_X1_1 | Tissue |
| 3 | 151166337 | C | T | IGSF10 | p.D478N | 0 | 82 | 0.12069 | 58 | WCM213_X1_1 | Tissue |
| 3 | 49720558 | A | G | APEH | p.K691R | 0 | 188 | 0.136364 | 110 | WCM213_X1_1 | Tissue |
| 3 | 52185079 | C | T | POC1A | p.R19Q | 0 | 89 | 0.082353 | 85 | WCM213_X1_1 | Tissue |
| 3 | 78710186 | C | G | ROBO1 | p.E772Q | 0 | 71 | 0.152941 | 85 | WCM213_X1_1 | Tissue |
| 4 | 5468549 | C | G | STK32B | p.D343E | 0 | 32 | 0.071429 | 42 | WCM213_X1_1 | Tissue |
| 4 | 6083352 | C | T | JAKMIP1 | p.R362Q | 0 | 81 | 0.078947 | 76 | WCM213_X1_1 | Tissue |
| 4 | 73184410 | C | T | ADAMTS3 | p.C455Y | 0 | 94 | 0.155844 | 77 | WCM213_X1_1 | Tissue |
| 5 | 154181813 | C | T | LARP1 | p.R578W | 0 | 52 | 0.108696 | 46 | WCM213_X1_1 | Tissue |
| 5 | 156899352 | T | G | NIPAL4 | p.F262C | 0 | 81 | 0.093333 | 75 | WCM213_X1_1 | Tissue |
| 5 | 35875633 | C | A | IL7R | p.P274T | 0 | 186 | 0.051852 | 135 | WCM213_X1_1 | Tissue |
| 5 | 38493725 | T | C | LIFR | p.E683G | 0 | 72 | 0.084507 | 71 | WCM213_X1_1 | Tissue |
| 5 | 66462008 | C | G | MAST4 | p.P2334R | 0 | 219 | 0.167598 | 179 | WCM213_X1_1 | Tissue |
| 7 | 100210427 | G | C | MOSPD3 | p.A5P | 0 | 103 | 0.10084 | 119 | WCM213_X1_1 | Tissue |
| 7 | 99197857 | T | A | GS1- | p.F55I | 0 | 212 | 0.126126 | 222 | WCM213_X1_1 | Tissue |
| | | | | 259H13.10 | | | | | | | |
| 8 | 131848572 | C | T | ADCY8 | p.V876I | 0 | 179 | 0.326693 | 251 | WCM213_X1_1 | Tissue |
| 9 | 115422224 | C | T | KIAA1958 | p.R676C | 0 | 163 | 0.168539 | 89 | WCM213_X1_1 | Tissue |
| 9 | 35044247 | G | C | C9orf131 | p.A541P | 0 | 172 | 0.217822 | 101 | WCM213_X1_1 | Tissue |
| 9 | 37782031 | A | G | EXOSC3 | p.I193T | 0 | 233 | 0.109827 | 173 | WCM213_X1_1 | Tissue |
| X | 22292264 | G | T | ZNF645 | p.D386Y | 0 | 45 | 0.149254 | 67 | WCM213_X1_1 | Tissue |
| X | 53566676 | G | C | HUWE1 | p.D3858E | 0 | 44 | 0.246575 | 73 | WCM213_X1_1 | Tissue |
| X | 74332773 | G | T | ABCB7 | p.T94K | 0 | 81 | 0.070423 | 71 | WCM213_X1_1 | Tissue |
| 10 | 112541631 | T | C | RBM20 | p.F422L | 0 | 295 | 0.114068 | 263 | WCM213_Z13_1 | Tissue |
| 10 | 124345687 | A | T | DMBT1 | p.D524V | 0 | 107 | 0.083969 | 131 | WCM213_Z13_1 | Tissue |
| 10 | 47669115 | G | T | ANTXRL | p.G235C | 0 | 78 | 0.063636 | 110 | WCM213_Z13_1 | Tissue |
| 10 | 95993933 | G | A | PLCE1 | p.R693H | 0 | 63 | 0.076923 | 52 | WCM213_Z13_1 | Tissue |
| 1 | 109466823 | T | C | GPSM2 | p.L601P | 0 | 61 | 0.219512 | 41 | WCM213_Z13_1 | Tissue |
| 11 | 124875035 | C | T | CCDC15 | p.L780F | 0 | 135 | 0.091954 | 87 | WCM213_Z13_1 | Tissue |
| 11 | 1255834 | G | A | MUC5B | p.E852K | 0 | 173 | 0.080169 | 237 | WCM213_Z13_1 | Tissue |
| 11 | 36597306 | G | T | RAG1 | p.V818L | 0 | 145 | 0.103448 | 145 | WCM213_Z13_1 | Tissue |
| 11 | 36614203 | G | A | RAG2 | p.R506C | 0 | 117 | 0.150327 | 153 | WCM213_Z13_1 | Tissue |
| 1 | 149877492 | C | T | SV2A | p.S662N | 0 | 141 | 0.052632 | 114 | WCM213_Z13_1 | Tissue |
| 1 | 166974600 | G | T | MAEL | p.D271Y | 0 | 49 | 0.060606 | 66 | WCM213_Z13_1 | Tissue |
| 11 | 71259729 | G | A | KRTAP5-9 | p.G9D | 0 | 164 | 0.054795 | 146 | WCM213_Z13_1 | Tissue |
| 1 | 176992672 | C | G | ASTN1 | p.D436H | 0 | 183 | 0.060345 | 232 | WCM213_Z13_1 | Tissue |
| 1 | 185143976 | A | T | SWT1 | p.S233C | 0 | 35 | 0.181818 | 55 | WCM213_Z13_1 | Tissue |
| 11 | 92085484 | G | A | FAT3 | p.G69D | 0 | 49 | 0.27027 | 37 | WCM213_Z13_1 | Tissue |
| 12 | 102108390 | A | T | CHPT1 | p.Q177L | 0 | 43 | 0.125 | 32 | WCM213_Z13_1 | Tissue |
| 12 | 104179177 | G | C | NT5DC3 | p.T422R | 0 | 153 | 0.141892 | 148 | WCM213_Z13_1 | Tissue |
| 1 | 22902925 | C | G | EPHA8 | p.Y125* | 0 | 110 | 0.074324 | 148 | WCM213_Z13_1 | Tissue |
| 12 | 49391127 | T | C | DDN | p.Q511R | 0 | 145 | 0.07619 | 105 | WCM213_Z13_1 | Tissue |
| 12 | 58020708 | C | G | B4GALNT1 | p.G474A | 0 | 151 | 0.0625 | 112 | WCM213_Z13_1 | Tissue |
| 12 | 7287982 | G | T | CLSTN3 | p.R148L | 0 | 215 | 0.097561 | 164 | WCM213_Z13_1 | Tissue |
| 12 | 91445224 | G | A | KERA | p.P320S | 0 | 170 | 0.0875 | 240 | WCM213_Z13_1 | Tissue |
| 13 | 39438625 | C | T | FREM2 | p.T2622I | 0 | 81 | 0.08 | 75 | WCM213_Z13_1 | Tissue |
| 1 | 42047861 | C | G | HIVEP3 | p.E870Q | 0 | 116 | 0.075 | 120 | WCM213_Z13_1 | Tissue |
| 14 | 21860859 | G | C | CHD8 | p.P2193R | 0 | 146 | 0.126316 | 95 | WCM213_Z13_1 | Tissue |
| 1 | 46740310 | C | A | RAD54L | p.P597Q | 0 | 123 | 0.084034 | 119 | WCM213_Z13_1 | Tissue |
| 15 | 41146858 | T | C | SPINT1 | p.L379P | 0 | 126 | 0.124224 | 161 | WCM213_Z13_1 | Tissue |
| 15 | 50534866 | T | A | HDC | p.Q527L | 0 | 129 | 0.125874 | 143 | WCM213_Z13_1 | Tissue |
| 15 | 50555481 | T | G | HDC | p.D52A | 0 | 197 | 0.073171 | 82 | WCM213_Z13_1 | Tissue |
| 16 | 14721013 | A | G | PARN | p.F93L | 0 | 230 | 0.274286 | 350 | WCM213_Z13_1 | Tissue |
| 16 | 30409820 | C | G | ZNF48 | p.P417A | 0 | 70 | 0.05814 | 86 | WCM213_Z13_1 | Tissue |
| 16 | 57741445 | A | C | CCDC135 | p.K311T | 0 | 80 | 0.061856 | 97 | WCM213_Z13_1 | Tissue |
| 1 | 68512503 | C | G | DIRAS3 | p.E160Q | 0 | 103 | 0.055556 | 90 | WCM213_Z13_1 | Tissue |
| 17 | 2996057 | G | C | OR1D2 | p.I78M | 0 | 104 | 0.160377 | 106 | WCM213_Z13_1 | Tissue |
| 17 | 53045790 | C | A | COX11 | p.R73L | 0 | 109 | 0.089041 | 146 | WCM213_Z13_1 | Tissue |
| 18 | 5445164 | G | A | EPB41L3 | p.T154M | 0 | 34 | 0.38983 | 59 | WCM213_Z13_1 | Tissue |
| 19 | 14938182 | C | G | OR7A5 | p.S291T | 0 | 95 | 0.117647 | 85 | WCM213_Z13_1 | Tissue |
| 19 | 15769192 | C | T | CYP4F3 | p.R412W | 0 | 111 | 0.066667 | 120 | WCM213_Z13_1 | Tissue |
| 19 | 2438190 | G | C | LMNB2 | p.L199V | 0 | 155 | 0.06383 | 94 | WCM213_Z13_1 | Tissue |
| 19 | 42909569 | C | T | LIPE | p.R837H | 0 | 62 | 0.112903 | 62 | WCM213_Z13_1 | Tissue |
| 19 | 48047729 | G | C | ZNF541 | p.T686S | 0 | 39 | 0.130435 | 46 | WCM213_Z13_1 | Tissue |
| 19 | 55672534 | G | A | DNAAF3 | p.R271C | 0 | 45 | 0.135135 | 37 | WCM213_Z13_1 | Tissue |
| 19 | 55687474 | C | G | SYT5 | p.E91Q | 0 | 123 | 0.072464 | 138 | WCM213_Z13_1 | Tissue |
| 20 | 25203492 | T | C | ENTPD6 | p.L355P | 0 | 143 | 0.060976 | 164 | WCM213_Z13_1 | Tissue |
| 20 | 55968347 | G | C | RBM38 | p.G125A | 0 | 136 | 0.055556 | 162 | WCM213_Z13_1 | Tissue |
| 20 | 56078638 | G | A | CTCFL | p.S565L | 0 | 153 | 0.070234 | 299 | WCM213_Z13_1 | Tissue |
| 2 | 113591074 | C | A | IL1B | p.G60C | 0 | 238 | 0.148594 | 249 | WCM213_Z13_1 | Tissue |
| 21 | 31654507 | G | C | KRTAP24-1 | p.S248R | 0 | 124 | 0.050562 | 178 | WCM213_Z13_1 | Tissue |
| 21 | 47821593 | G | C | PCNT | p.Q1640H | 0 | 245 | 0.120521 | 307 | WCM213_Z13_1 | Tissue |
| 2 | 207170837 | A | G | ZDBF2 | p.K529E | 0 | 112 | 0.114035 | 114 | WCM213_Z13_1 | Tissue |
| 2 | 208489076 | C | G | METTL21A | p.E8D | 0 | 75 | 0.49 | 100 | WCM213_Z13_1 | Tissue |
| 2 | 20974602 | G | C | C2orf43 | p.L146V | 0 | 39 | 0.109091 | 55 | WCM213_Z13_1 | Tissue |
| 22 | 24581790 | A | G | SUSD2 | p.Y411C | 0 | 140 | 0.140625 | 128 | WCM213_Z13_1 | Tissue |
| 22 | 30888539 | T | C | SEC14L4 | p.K196E | 0 | 96 | 0.094828 | 116 | WCM213_Z13_1 | Tissue |
| 22 | 40718868 | G | T | TNRC6B | p.G1709* | 0 | 163 | 0.058824 | 170 | WCM213_Z13_1 | Tissue |
| 2 | 64147683 | C | T | VPS54 | p.E650K | 0 | 81 | 0.230769 | 39 | WCM213_Z13_1 | Tissue |
| 3 | 119013771 | A | T | ARHGAP31 | p.K7M | 0 | 72 | 0.054264 | 129 | WCM213_Z13_1 | Tissue |
| 3 | 119013772 | G | T | ARHGAP31 | p.K7N | 0 | 71 | 0.054264 | 129 | WCM213_Z13_1 | Tissue |
| 3 | 124103725 | G | A | KALRN | p.A600T | 0 | 88 | 0.098039 | 102 | WCM213_Z13_1 | Tissue |
| 3 | 151165013 | C | T | IGSF10 | p.R919K | 0 | 114 | 0.128205 | 78 | WCM213_Z13_1 | Tissue |
| 3 | 151166136 | C | T | IGSF10 | p.D545N | 0 | 109 | 0.1 | 70 | WCM213_Z13_1 | Tissue |
| 3 | 151166337 | C | T | IGSF10 | p.D478N | 0 | 82 | 0.098765 | 81 | WCM213_Z13_1 | Tissue |
| 3 | 33438622 | C | T | UBP1 | p.D396N | 0 | 73 | 0.061947 | 113 | WCM213_Z13_1 | Tissue |
| 3 | 39130778 | T | A | WDR48 | p.V546E | 0 | 56 | 0.083333 | 72 | WCM213_Z13_1 | Tissue |
| 3 | 49720558 | A | G | APEH | p.K691R | 0 | 188 | 0.303226 | 155 | WCM213_Z13_1 | Tissue |
| 3 | 50412231 | G | A | CACNA2D2 | p.L641F | 0 | 114 | 0.078947 | 76 | WCM213_Z13_1 | Tissue |
| 3 | 78710186 | C | G | ROBO1 | p.E772Q | 0 | 71 | 0.144928 | 69 | WCM213_Z13_1 | Tissue |
| 4 | 38995570 | G | A | TMEM156 | p.S136L | 0 | 75 | 0.220588 | 68 | WCM213_Z13_1 | Tissue |
| 5 | 140250171 | G | C | PCDHA11 | p.E495Q | 0 | 83 | 0.081081 | 37 | WCM213_Z13_1 | Tissue |
| 5 | 156899352 | T | G | NIPAL4 | p.F262C | 0 | 81 | 0.089431 | 123 | WCM213_Z13_1 | Tissue |
| 6 | 43030670 | G | T | KLC4 | p.A92S | 0 | 34 | 0.135135 | 37 | WCM213_Z13_1 | Tissue |
| 7 | 100210427 | G | C | MOSPD3 | p.A5P | 0 | 103 | 0.209302 | 129 | WCM213_Z13_1 | Tissue |
| 7 | 21920465 | C | A | DNAH11 | p.T4114N | 0 | 94 | 0.082353 | 85 | WCM213_Z13_1 | Tissue |
| 7 | 28547258 | T | A | CREB5 | p.F65Y | 0 | 175 | 0.051852 | 135 | WCM213_Z13_1 | Tissue |
| 7 | 73151347 | G | C | ABHD11 | p.L243V | 0 | 54 | 0.056075 | 107 | WCM213_Z13_1 | Tissue |
| 7 | 98638151 | C | A | SMURF1 | p.G493V | 0 | 223 | 0.082353 | 255 | WCM213_Z13_1 | Tissue |
| 8 | 12956025 | G | T | DLC1 | p.S1017* | 0 | 84 | 0.148936 | 47 | WCM213_Z13_1 | Tissue |
| 8 | 131848572 | C | T | ADCY8 | p.V876I | 0 | 179 | 0.335294 | 340 | WCM213_Z13_1 | Tissue |
| 8 | 142204286 | G | C | DENND3 | p.G1184A | 0 | 198 | 0.157895 | 171 | WCM213_Z13_1 | Tissue |
| 8 | 144696811 | C | G | TSTA3 | p.M149I | 0 | 102 | 0.051948 | 154 | WCM213_Z13_1 | Tissue |
| 8 | 38205084 | C | G | WHSC1L1 | p.K202N | 0 | 109 | 0.075758 | 198 | WCM213_Z13_1 | Tissue |
| 9 | 115422224 | C | T | KIAA1958 | p.R676C | 0 | 163 | 0.333333 | 117 | WCM213_Z13_1 | Tissue |
| 9 | 35044247 | G | C | C9orf131 | p.A541P | 0 | 172 | 0.295775 | 213 | WCM213_Z13_1 | Tissue |
| 9 | 43627264 | G | A | SPATA31A6 | p.R475C | 0 | 137 | 0.090278 | 144 | WCM213_Z13_1 | Tissue |
| X | 22292264 | G | T | ZNF645 | p.D386Y | 0 | 45 | 0.228571 | 70 | WCM213_Z13_1 | Tissue |
| X | 591805 | A | G | SHOX | p.D58G | 0 | 100 | 0.079268 | 164 | WCM213_Z13_1 | Tissue |
| 1 | 2435680 | G | T | PLCH2 | p.R1093S | 0 | 35 | 0.085714 | 35 | WCM216 | Plasma |
| 4 | 154556695 | G | T | KIAA0922 | p.W1509L | 0 | 43 | 0.070175 | 57 | WCM216 | Plasma |
| 1 | 159784043 | G | T | FCRL6 | p.V370F | 0 | 143 | 0.052632 | 152 | WCM216_Z1_1 | Tissue |
| 11 | 73104941 | C | T | RELT | p.R229C | 0 | 75 | 0.076923 | 78 | WCM216_Z1_1 | Tissue |
| 4 | 88016069 | C | A | AFF1 | p.Q404K | 0 | 67 | 0.086957 | 69 | WCM216_Z1_1 | Tissue |
| 7 | 56156627 | C | T | PHKG1 | p.R3Q | 0 | 45 | 0.075 | 40 | WCM216_Z1_1 | Tissue |
| 11 | 125267864 | G | T | PKNOX2 | p.C165F | 0 | 34 | 0.075 | 40 | WCM218 | Plasma |
| 15 | 43769897 | C | A | TP53BP1 | p.Q278H | 0 | 43 | 0.063291 | 79 | WCM218 | Plasma |
| 15 | 43783909 | C | G | TP53BP1 | p.S105T | 0 | 46 | 0.096154 | 52 | WCM218 | Plasma |
| 15 | 43783910 | T | C | TP53BP1 | p.S105G | 0 | 46 | 0.1 | 50 | WCM218 | Plasma |
| 10 | 70229791 | G | C | DNA2 | p.I68M | 0 | 73 | 0.321429 | 56 | WCM218_Z11_1 | Tissue |
| 11 | 1101093 | A | G | MUC2 | p.T2498A | 0 | 65 | 0.196429 | 56 | WCM218_Z11_1 | Tissue |
| 11 | 118769761 | C | T | BCL9L | p.R1288H | 0 | 139 | 0.25 | 116 | WCM218_Z11_1 | Tissue |
| 11 | 33589733 | G | A | KIAA1549L | p.R110OQ | 0 | 141 | 0.272727 | 176 | WCM218_Z11_1 | Tissue |
| 11 | 3661512 | C | A | ART5 | p.K49N | 0 | 69 | 0.2 | 110 | WCM218_Z11_1 | Tissue |
| 11 | 46564357 | A | T | AMBRA1 | p.S404T | 0 | 163 | 0.2657 | 207 | WCM218_Z11_1 | Tissue |
| 1 | 21053505 | C | T | SH2D5 | p.G78R | 0 | 59 | 0.060606 | 66 | WCM218_Z11_1 | Tissue |
| 12 | 113737742 | C | A | SLC8B1 | p.G532V | 0 | 245 | 0.374016 | 254 | WCM218_Z11_1 | Tissue |
| 12 | 29640802 | C | G | OVCH1 | p.G239A | 0 | 38 | 0.171429 | 35 | WCM218_Z11_1 | Tissue |
| 12 | 55357585 | A | G | TESPA1 | p.L199P | 0 | 97 | 0.218543 | 151 | WCM218_Z11_1 | Tissue |
| 12 | 57601911 | G | A | LRP1 | p.V3984M | 0 | 80 | 0.082353 | 85 | WCM218_Z11_1 | Tissue |
| 12 | 6637175 | G | C | NCAPD2 | p.V1021L | 0 | 153 | 0.152381 | 105 | WCM218_Z11_1 | Tissue |
| 14 | 38091823 | G | A | TTC6 | p.R185H | 0 | 55 | 0.283019 | 53 | WCM218_Z11_1 | Tissue |
| 1 | 51871579 | G | C | EPS15 | p.P559A | 0 | 273 | 0.092437 | 238 | WCM218_Z11_1 | Tissue |
| 15 | 76018504 | C | G | ODF3L1 | p.S112C | 0 | 136 | 0.06962 | 158 | WCM218_Z11_1 | Tissue |
| 16 | 3406074 | T | A | OR2C1 | p.I45N | 0 | 79 | 0.235849 | 106 | WCM218_Z11_1 | Tissue |
| 16 | 58619368 | A | T | CNOT1 | p.L227* | 0 | 106 | 0.075758 | 66 | WCM218_Z11_1 | Tissue |
| 17 | 19470440 | G | T | SLC47A1 | p.G403V | 0 | 263 | 0.238462 | 130 | WCM218_Z11_1 | Tissue |
| 17 | 27208908 | T | C | FLOT2 | p.K280E | 0 | 173 | 0.178571 | 140 | WCM218_Z11_1 | Tissue |
| 17 | 3352424 | T | A | SPATA22 | p.N117Y | 0 | 103 | 0.365385 | 104 | WCM218_Z11_1 | Tissue |
| 17 | 78313184 | C | G | RNF213 | p.L1673V | 0 | 111 | 0.22973 | 148 | WCM218_Z11_1 | Tissue |
| 17 | 7951863 | G | A | ALOX15B | p.E671K | 0 | 135 | 0.306122 | 98 | WCM218_Z11_1 | Tissue |
| 19 | 17323072 | C | T | MYO9B | p.P2143S | 0 | 36 | 0.212121 | 33 | WCM218_Z11_1 | Tissue |
| 19 | 17688896 | G | A | COLGALT1 | p.E422K | 0 | 105 | 0.293578 | 109 | WCM218_Z11_1 | Tissue |
| 19 | 53612278 | C | A | ZNF415 | p.R340S | 0 | 88 | 0.211111 | 90 | WCM218_Z11_1 | Tissue |
| 19 | 6935074 | C | A | EMR1 | p.T789K | 0 | 114 | 0.254902 | 102 | WCM218_Z11_1 | Tissue |
| 20 | 44641941 | C | T | MMP9 | p.Q460* | 0 | 138 | 0.204762 | 210 | WCM218_Z11_1 | Tissue |
| 2 | 112939442 | G | C | FBLN7 | p.S220T | 0 | 119 | 0.410526 | 95 | WCM218_Z11_1 | Tissue |
| 2 | 128932419 | T | C | UGGT1 | p.Y1176H | 0 | 61 | 0.163462 | 104 | WCM218_Z11_1 | Tissue |
| 2 | 225639690 | G | T | DOCK10 | p.P1982H | 0 | 48 | 0.178571 | 84 | WCM218_Z11_1 | Tissue |
| 3 | 115561378 | G | A | LSAMP | p.R233* | 0 | 92 | 0.2 | 65 | WCM218_Z11_1 | Tissue |
| 3 | 38153837 | C | A | DLEC1 | p.D1217E | 0 | 264 | 0.141689 | 367 | WCM218_Z11_1 | Tissue |
| 3 | 48617487 | G | T | COL7A1 | p.P1701T | 0 | 75 | 0.283333 | 60 | WCM218_Z11_1 | Tissue |
| 3 | 49166488 | G | T | LAMB2 | p.H566N | 0 | 52 | 0.21875 | 32 | WCM218_Z11_1 | Tissue |
| 3 | 50685395 | C | T | MAPKAPK3 | p.T356I | 0 | 208 | 0.272727 | 187 | WCM218_Z11_1 | Tissue |
| 3 | 77666743 | G | T | ROBO2 | p.D1125Y | 0 | 124 | 0.253521 | 142 | WCM218_Z11_1 | Tissue |
| 4 | 119252953 | G | T | PRSS12 | p.H297N | 0 | 179 | 0.34728 | 239 | WCM218_Z11_1 | Tissue |
| 4 | 141600909 | C | A | TBC1D9 | p.R150M | 0 | 110 | 0.345238 | 168 | WCM218_Z11_1 | Tissue |
| 5 | 180047942 | G | A | FLT4 | p.R745C | 0 | 40 | 0.130435 | 46 | WCM218_Z11_1 | Tissue |
| 6 | 30672288 | C | A | MDC1 | p.G1558C | 0 | 225 | 0.061111 | 180 | WCM218_Z11_1 | Tissue |
| 7 | 149129873 | G | A | ZNF777 | p.P497L | 0 | 125 | 0.254545 | 55 | WCM218_Z11_1 | Tissue |
| 7 | 155301708 | C | G | CNPY1 | p.D9H | 0 | 125 | 0.297778 | 225 | WCM218_Z11_1 | Tissue |
| 7 | 37960642 | G | T | EPDR1 | p.G34V | 0 | 31 | 0.104167 | 48 | WCM218_Z11_1 | Tissue |
| 8 | 101146260 | T | C | FBXO43 | p.T633A | 0 | 119 | 0.089888 | 89 | WCM218_Z11_1 | Tissue |
| 8 | 104898333 | G | C | RIMS2 | p.K280N | 0 | 74 | 0.197802 | 91 | WCM218_Z11_1 | Tissue |
| 8 | 106813685 | A | G | ZFPM2 | p.T459A | 0 | 112 | 0.44186 | 86 | WCM218_Z11_1 | Tissue |
| 8 | 107782084 | G | A | ABRA | p.T112M | 0 | 64 | 0.324561 | 114 | WCM218_Z11_1 | Tissue |
| 8 | 67577813 | C | A | VCPIP1 | p.V461L | 0 | 88 | 0.212121 | 99 | WCM218_Z11_1 | Tissue |
| 9 | 125377338 | G | A | OR1Q1 | p.A108T | 0 | 112 | 0.102041 | 98 | WCM218_Z11_1 | Tissue |
| 9 | 97082767 | T | C | NUTM2F | p.Q364R | 0 | 59 | 0.123077 | 130 | WCM218_Z11_1 | Tissue |
| X | 130408142 | G | A | IGSF1 | p.R1264* | 0 | 71 | 0.583333 | 108 | WCM218_Z11_1 | Tissue |
| 10 | 70229791 | G | C | DNA2 | p.I68M | 0 | 73 | 0.2 | 75 | WCM218_Z3_1 | Tissue |
| 11 | 103797726 | G | A | PDGFD | p.R301C | 0 | 103 | 0.330579 | 121 | WCM218_Z3_1 | Tissue |
| 11 | 1101093 | A | G | MUC2 | p.T2498A | 0 | 65 | 0.371429 | 35 | WCM218_Z3_1 | Tissue |
| 11 | 118769761 | C | T | BCL9L | p.R1288H | 0 | 139 | 0.346457 | 127 | WCM218_Z3_1 | Tissue |
| 11 | 33589733 | G | A | KIAA1549L | p.R110OQ | 0 | 141 | 0.373913 | 115 | WCM218_Z3_1 | Tissue |
| 11 | 3661512 | C | A | ART5 | p.K49N | 0 | 69 | 0.189474 | 95 | WCM218_Z3_1 | Tissue |
| 11 | 46564357 | A | T | AMBRA1 | p.S404T | 0 | 163 | 0.293706 | 143 | WCM218_Z3_1 | Tissue |
| 1 | 182845272 | G | A | DHX9 | p.E635K | 0 | 31 | 0.162791 | 43 | WCM218_Z3_1 | Tissue |
| 12 | 113737742 | C | A | SLC8B1 | p.G532V | 0 | 245 | 0.458194 | 299 | WCM218_Z3_1 | Tissue |
| 12 | 29640802 | C | G | OVCH1 | p.G239A | 0 | 38 | 0.229167 | 48 | WCM218_Z3_1 | Tissue |
| 1 | 234744504 | G | A | IRF2BP2 | p.A246V | 0 | 57 | 0.084746 | 59 | WCM218_Z3_1 | Tissue |
| 12 | 55357585 | A | G | TESPA1 | p.L199P | 0 | 97 | 0.130841 | 107 | WCM218_Z3_1 | Tissue |
| 1 | 37346322 | C | T | GRIK3 | p.D155N | 0 | 51 | 0.341463 | 41 | WCM218_Z3_1 | Tissue |
| 14 | 38091823 | G | A | TTC6 | p.R185H | 0 | 55 | 0.234043 | 47 | WCM218_Z3_1 | Tissue |
| 1 | 51871579 | G | C | EPS15 | p.P559A | 0 | 273 | 0.174699 | 166 | WCM218_Z3_1 | Tissue |
| 15 | 76018504 | C | G | ODF3L1 | p.S112C | 0 | 136 | 0.164835 | 91 | WCM218_Z3_1 | Tissue |
| 16 | 3406074 | T | A | OR2C1 | p.I45N | 0 | 79 | 0.213333 | 75 | WCM218_Z3_1 | Tissue |
| 16 | 58619317 | C | G | CNOT1 | p.G244A | 0 | 43 | 0.078431 | 51 | WCM218_Z3_1 | Tissue |
| 16 | 58619368 | A | T | CNOT1 | p.L227* | 0 | 106 | 0.08 | 100 | WCM218_Z3_1 | Tissue |
| 16 | 67655363 | G | A | CTCF | p.C409Y | 0 | 191 | 0.055866 | 179 | WCM218_Z3_1 | Tissue |
| 17 | 19470440 | G | T | SLC47A1 | p.G403V | 0 | 263 | 0.275109 | 229 | WCM218_Z3_1 | Tissue |
| 17 | 3352424 | T | A | SPATA22 | p.N117Y | 0 | 103 | 0.357895 | 95 | WCM218_Z3_1 | Tissue |
| 17 | 78313184 | C | G | RNF213 | p.L1673V | 0 | 111 | 0.266187 | 139 | WCM218_Z3_1 | Tissue |
| 17 | 7951863 | G | A | ALOX15B | p.E671K | 0 | 135 | 0.259259 | 81 | WCM218_Z3_1 | Tissue |
| 19 | 17323072 | C | T | MYO9B | p.P2143S | 0 | 36 | 0.205128 | 39 | WCM218_Z3_1 | Tissue |
| 19 | 17688896 | G | A | COLGALT1 | p.E422K | 0 | 105 | 0.241758 | 91 | WCM218_Z3_1 | Tissue |
| 19 | 41179380 | C | T | NUMBL | p.R302H | 0 | 61 | 0.0625 | 96 | WCM218_Z3_1 | Tissue |
| 19 | 53612278 | C | A | ZNF415 | p.R340S | 0 | 88 | 0.326087 | 92 | WCM218_Z3_1 | Tissue |
| 19 | 6935074 | C | A | EMR1 | p.T789K | 0 | 114 | 0.413793 | 58 | WCM218_Z3_1 | Tissue |
| 20 | 44641941 | C | T | MMP9 | p.Q460* | 0 | 138 | 0.24812 | 133 | WCM218_Z3_1 | Tissue |
| 2 | 112939442 | G | C | FBLN7 | p.S220T | 0 | 119 | 0.421053 | 95 | WCM218_Z3_1 | Tissue |
| 2 | 128932419 | T | C | UGGT1 | p.Y1176H | 0 | 61 | 0.294118 | 68 | WCM218_Z3_1 | Tissue |
| 2 | 225639690 | G | T | DOCK10 | p.P1982H | 0 | 48 | 0.418182 | 55 | WCM218_Z3_1 | Tissue |
| 3 | 115561378 | G | A | LSAMP | p.R233* | 0 | 92 | 0.273684 | 95 | WCM218_Z3_1 | Tissue |
| 3 | 48617487 | G | T | COL7A1 | p.P1701T | 0 | 75 | 0.169492 | 59 | WCM218_Z3_1 | Tissue |
| 3 | 50685395 | C | T | MAPKAPK3 | p.T356I | 0 | 208 | 0.238095 | 189 | WCM218_Z3_1 | Tissue |
| 3 | 53857923 | C | T | CHDH | p.R38Q | 0 | 47 | 0.282051 | 39 | WCM218_Z3_1 | Tissue |
| 3 | 77666743 | G | T | ROBO2 | p.D1125Y | 0 | 124 | 0.243243 | 111 | WCM218_Z3_1 | Tissue |
| 4 | 119252953 | G | T | PRSS12 | p.H297N | 0 | 179 | 0.414365 | 181 | WCM218_Z3_1 | Tissue |
| 4 | 141600909 | C | A | TBC1D9 | p.R150M | 0 | 110 | 0.31405 | 121 | WCM218_Z3_1 | Tissue |
| 5 | 140176608 | G | A | PCDHA2 | p.A687T | 0 | 78 | 0.333333 | 45 | WCM218_Z3_1 | Tissue |
| 5 | 150639352 | G | A | GM2A | p.D40N | 0 | 158 | 0.330357 | 112 | WCM218_Z3_1 | Tissue |
| 6 | 108645100 | G | A | LACE1 | p.V71I | 0 | 38 | 0.360656 | 61 | WCM218_Z3_1 | Tissue |
| 6 | 152754970 | G | T | SYNE1 | p.T1474N | 0 | 93 | 0.105263 | 57 | WCM218_Z3_1 | Tissue |
| 7 | 149129873 | G | A | ZNF777 | p.P497L | 0 | 125 | 0.219697 | 132 | WCM218_Z3_1 | Tissue |
| 7 | 155301708 | C | G | CNPY1 | p.D9H | 0 | 125 | 0.266667 | 195 | WCM218_Z3_1 | Tissue |
| 8 | 101146260 | T | C | FBXO43 | p.T633A | 0 | 119 | 0.177305 | 141 | WCM218_Z3_1 | Tissue |
| 8 | 104898333 | G | C | RIMS2 | p.K280N | 0 | 74 | 0.273973 | 73 | WCM218_Z3_1 | Tissue |
| 8 | 106813685 | A | G | ZFPM2 | p.T459A | 0 | 112 | 0.411348 | 141 | WCM218_Z3_1 | Tissue |
| 8 | 107782084 | G | A | ABRA | p.T112M | 0 | 64 | 0.227273 | 66 | WCM218_Z3_1 | Tissue |
| 8 | 145773124 | C | A | ARHGAP39 | p.S449I | 0 | 47 | 0.148148 | 54 | WCM218_Z3_1 | Tissue |
| 8 | 67577813 | C | A | VCPIP1 | p.V461L | 0 | 88 | 0.186916 | 107 | WCM218_Z3_1 | Tissue |
| 8 | 82606504 | G | C | SLC10A5 | p.A235G | 0 | 67 | 0.2 | 80 | WCM218_Z3_1 | Tissue |
| 9 | 118950348 | G | A | PAPPA | p.R444H | 0 | 116 | 0.139344 | 122 | WCM218_Z3_1 | Tissue |
| 9 | 72892345 | C | T | SMC5 | p.A167V | 0 | 38 | 0.262295 | 61 | WCM218_Z3_1 | Tissue |
| 9 | 97082767 | T | C | NUTM2F | p.Q364R | 0 | 59 | 0.181818 | 77 | WCM218_Z3_1 | Tissue |
| X | 130408142 | G | A | IGSF1 | p.R1264* | 0 | 71 | 0.46 | 50 | WCM218_Z3_1 | Tissue |
| X | 38031221 | A | G | SRPX | p.C147R | 0 | 95 | 0.069444 | 72 | WCM218_Z3_1 | Tissue |
| 15 | 43748917 | C | G | TP53BP1 | p.S625T | 0 | 41 | 0.090909 | 44 | WCM219 | Plasma |
| 15 | 43748918 | T | C | TP53BP1 | p.S625G | 0 | 41 | 0.093023 | 43 | WCM219 | Plasma |
| 2 | 191382331 | G | T | TMEM194B | p.T176N | 0 | 54 | 0.088235 | 34 | WCM219 | Plasma |
| 15 | 43712787 | T | C | TP53BP1 | p.D1461G | 0 | 34 | 0.076923 | 39 | WCM223 | Plasma |
| 10 | 101716269 | G | A | DNMBP | p.P321L | 0 | 140 | 0.09375 | 64 | WCM223_Z2_1 | Tissue |
| 10 | 52103403 | C | T | SGMS1 | p.E158K | 0 | 166 | 0.054795 | 146 | WCM223_Z2_1 | Tissue |
| 1 | 10725506 | G | A | CASZ1 | p.R47* | 0 | 76 | 0.106061 | 66 | WCM223_Z2_1 | Tissue |
| 11 | 118211295 | C | A | CD3D | p.K23N | 0 | 59 | 0.191176 | 68 | WCM223_Z2_1 | Tissue |
| 1 | 120437527 | G | T | ADAM30 | p.P478Q | 0 | 80 | 0.095238 | 63 | WCM223_Z2_1 | Tissue |
| 1 | 181687235 | T | C | CACNA1E | p.S524P | 0 | 60 | 0.403226 | 62 | WCM223_Z2_1 | Tissue |
| 12 | 50747833 | C | G | FAM186A | p.G928R | 0 | 115 | 0.333333 | 63 | WCM223_Z2_1 | Tissue |
| 14 | 38061191 | G | C | FOXA1 | p.F266L | 0 | 67 | 0.571429 | 63 | WCM223_Z2_1 | Tissue |
| 1 | 46660039 | G | T | POMGNT1 | p.N262K | 0 | 95 | 0.060606 | 66 | WCM223_Z2_1 | Tissue |
| 15 | 49031309 | C | A | CEP152 | p.E1424* | 0 | 42 | 0.5 | 46 | WCM223_Z2_1 | Tissue |
| 1 | 66723353 | C | G | PDE4B | p.T167S | 0 | 74 | 0.070588 | 85 | WCM223_Z2_1 | Tissue |
| 17 | 19284298 | G | A | MAPK7 | p.R259H | 0 | 96 | 0.402985 | 67 | WCM223_Z2_1 | Tissue |
| 1 | 75037558 | G | A | C1orf173 | p.P1279L | 0 | 87 | 0.253521 | 71 | WCM223_Z2_1 | Tissue |
| 20 | 2464746 | G | T | ZNF343 | p.H287Q | 0 | 85 | 0.086957 | 69 | WCM223_Z2_1 | Tissue |
| 21 | 43166326 | C | T | RIPK4 | p.G280R | 0 | 95 | 0.393443 | 61 | WCM223_Z2_1 | Tissue |
| 2 | 179486277 | C | T | TTN | p.A13451T | 0 | 49 | 0.8 | 35 | WCM223_Z2_1 | Tissue |
| 4 | 158254023 | G | A | GRIA2 | p.R312H | 0 | 248 | 0.387931 | 116 | WCM223_Z2_1 | Tissue |
| 4 | 158257722 | G | C | GRIA2 | p.G556A | 0 | 153 | 0.649123 | 57 | WCM223_Z2_1 | Tissue |
| 5 | 96126063 | G | C | ERAP1 | p.P487R | 0 | 84 | 0.560976 | 82 | WCM223_Z2_1 | Tissue |
| 6 | 152461140 | C | T | SYNE1 | p.R8468H | 0 | 101 | 0.132075 | 53 | WCM223_Z2_1 | Tissue |
| 6 | 34029684 | G | T | GRM4 | p.N286K | 0 | 106 | 0.075 | 80 | WCM223_Z2_1 | Tissue |
| 7 | 82583461 | C | A | PCLO | p.A2270S | 0 | 33 | 0.076923 | 39 | WCM223_Z2_1 | Tissue |
| 8 | 132052522 | G | T | ADCY8 | p.P20T | 0 | 124 | 0.536585 | 41 | WCM223_Z2_1 | Tissue |
| 8 | 133650275 | T | C | LRRC6 | p.N112S | 0 | 134 | 0.3 | 50 | WCM223_Z2_1 | Tissue |
| 8 | 21983191 | C | T | HR | p.C487Y | 0 | 217 | 0.467626 | 139 | WCM223_Z2_1 | Tissue |
| 8 | 89128849 | T | G | MMP16 | p.K324Q | 0 | 61 | 0.092308 | 65 | WCM223_Z2_1 | Tissue |
| X | 142967267 | G | C | UBE2NL | p.G22A | 0 | 53 | 0.942857 | 35 | WCM223_Z2_1 | Tissue |
| X | 69368672 | G | T | IGBP1 | p.R247L | 0 | 67 | 0.066667 | 45 | WCM223_Z2_1 | Tissue |
| 11 | 108382276 | A | G | EXPH5 | p.S1320P | 0 | 90 | 0.85 | 60 | WCM223_Z4_1 | Tissue |
| 1 | 158576737 | G | T | OR10Z1 | p.S170I | 0 | 343 | 0.29703 | 202 | WCM223_Z4_1 | Tissue |
| 11 | 76868342 | C | A | MYO7A | p.N251K | 0 | 264 | 0.428571 | 238 | WCM223_Z4_1 | Tissue |
| 12 | 50392983 | T | G | RACGAP1 | p.S274R | 0 | 134 | 0.368932 | 103 | WCM223_Z4_1 | Tissue |
| 1 | 3644315 | C | A | TP73 | p.N322K | 0 | 92 | 0.081081 | 74 | WCM223_Z4_1 | Tissue |
| 1 | 43634636 | G | A | EBNA1BP2 | p.R171* | 0 | 258 | 0.564246 | 179 | WCM223_Z4_1 | Tissue |
| 17 | 19284298 | G | A | MAPK7 | p.R259H | 0 | 169 | 0.404762 | 126 | WCM223_Z4_1 | Tissue |
| 17 | 34934417 | T | C | GGNBP2 | p.C216R | 0 | 70 | 0.4 | 80 | WCM223_Z4_1 | Tissue |
| 18 | 8784557 | G | A | SOGA2 | p.G843R | 0 | 172 | 0.426829 | 164 | WCM223_Z4_1 | Tissue |
| 2 | 128865631 | G | A | UGGT1 | p.A133T | 0 | 114 | 0.111111 | 54 | WCM223_Z4_1 | Tissue |
| 21 | 45993654 | G | A | KRTAP10-4 | p.D7N | 0 | 157 | 0.490566 | 106 | WCM223_Z4_1 | Tissue |
| 2 | 69093011 | C | T | BMP10 | p.A343T | 0 | 44 | 0.452991 | 117 | WCM223_Z4_1 | Tissue |
| 4 | 158254023 | G | A | GRIA2 | p.R312H | 0 | 174 | 0.494737 | 190 | WCM223_Z4_1 | Tissue |
| 5 | 73197014 | G | T | ARHGEF28 | p.V1327F | 0 | 162 | 0.089552 | 67 | WCM223_Z4_1 | Tissue |
| 8 | 132052522 | G | T | ADCY8 | p.P20T | 0 | 222 | 0.49635 | 137 | WCM223_Z4_1 | Tissue |
| 8 | 87162356 | C | T | ATP6V0D2 | p.R219C | 0 | 80 | 0.347826 | 92 | WCM223_Z4_1 | Tissue |
| X | 142967267 | G | C | UBE2NL | p.G22A | 0 | 46 | 0.942308 | 52 | WCM223_Z4_1 | Tissue |
| 19 | 49238557 | C | T | RASIP1 | p.G359R | 0 | 36 | 0.095745 | 94 | WCM243 | Plasma |
| 1 | 152275808 | C | G | FLG | p.G3852R | 0 | 68 | 0.215909 | 88 | WCM243_X1_1 | Tissue |
| 11 | 55927030 | A | C | OR8K5 | p.L255R | 0 | 172 | 0.105991 | 217 | WCM243_X1_1 | Tissue |
| 11 | 56000198 | G | T | OR5T2 | p.A155D | 0 | 81 | 0.12766 | 94 | WCM243_X1_1 | Tissue |
| 11 | 6898584 | C | T | OR10A4 | p.H236Y | 0 | 127 | 0.331034 | 145 | WCM243_X1_1 | Tissue |
| 12 | 114836461 | C | T | TBX5 | p.A143T | 0 | 143 | 0.374408 | 211 | WCM243_X1_1 | Tissue |
| 12 | 121176678 | G | A | ACADS | p.R330H | 0 | 162 | 0.151515 | 198 | WCM243_X1_1 | Tissue |
| 1 | 231334860 | G | A | TRIM67 | p.R403H | 0 | 101 | 0.276596 | 94 | WCM243_X1_1 | Tissue |
| 14 | 105404579 | G | A | AHNAK2 | p.R5737* | 0 | 199 | 0.165854 | 205 | WCM243_X1_1 | Tissue |
| 14 | 58909548 | G | A | KIAA0586 | p.V164M | 0 | 66 | 0.391304 | 46 | WCM243_X1_1 | Tissue |
| 15 | 85326151 | A | C | ZNF592 | p.E82A | 0 | 81 | 0.245614 | 57 | WCM243_X1_1 | Tissue |
| 15 | 86838635 | G | T | AGBL1 | p.Q744H | 0 | 219 | 0.051948 | 154 | WCM243_X1_1 | Tissue |
| 16 | 27709753 | G | T | KIAA0556 | p.V349L | 0 | 63 | 0.060606 | 66 | WCM243_X1_1 | Tissue |
| 16 | 3367592 | A | G | ZNF75A | p.N205S | 0 | 92 | 0.260274 | 73 | WCM243_X1_1 | Tissue |
| 17 | 650490 | A | G | GEMIN4 | p.Y265H | 0 | 197 | 0.099415 | 171 | WCM243_X1_1 | Tissue |
| 1 | 78178939 | A | C | USP33 | p.I779S | 0 | 32 | 0.142857 | 35 | WCM243_X1_1 | Tissue |
| 19 | 17598085 | C | T | SLC27A1 | p.P222L | 0 | 51 | 0.075472 | 53 | WCM243_X1_1 | Tissue |
| 19 | 49238557 | C | T | RASIP1 | p.G359R | 0 | 58 | 0.471698 | 53 | WCM243_X1_1 | Tissue |
| 20 | 57767362 | C | T | ZNF831 | p.R430W | 0 | 44 | 0.054795 | 73 | WCM243_X1_1 | Tissue |
| 2 | 179395177 | T | C | TTN | p.I33748V | 0 | 129 | 0.294643 | 112 | WCM243_X1_1 | Tissue |
| 2 | 20482930 | G | A | PUM2 | p.R500W | 0 | 109 | 0.313726 | 51 | WCM243_X1_1 | Tissue |
| 3 | 129269965 | G | T | H1FOO | p.A275S | 0 | 73 | 0.201923 | 104 | WCM243_X1_1 | Tissue |
| 3 | 37512512 | C | T | ITGA9 | p.A67V | 0 | 81 | 0.122222 | 90 | WCM243_X1_1 | Tissue |
| 3 | 49679897 | G | A | BSN | p.R277H | 0 | 33 | 0.275 | 40 | WCM243_X1_1 | Tissue |
| 4 | 100574365 | G | A | RP11- | p.P481S | 0 | 65 | 0.108696 | 46 | WCM243_X1_1 | Tissue |
| | | | | 766F14.2 | | | | | | | |
| 5 | 176700724 | T | A | NSD1 | p.L1854Q | 0 | 120 | 0.189024 | 164 | WCM243_X1_1 | Tissue |
| 6 | 139694804 | A | T | CITED2 | p.F93Y | 0 | 66 | 0.507246 | 69 | WCM243_X1_1 | Tissue |
| 7 | 128485276 | G | A | FLNC | p.V1253I | 0 | 131 | 0.141553 | 219 | WCM243_X1_1 | Tissue |
| 7 | 128490514 | C | T | FLNC | p.A1792V | 0 | 110 | 0.312139 | 173 | WCM243_X1_1 | Tissue |
| 7 | 148863929 | G | T | ZNF398 | p.D190Y | 0 | 79 | 0.168142 | 113 | WCM243_X1_1 | Tissue |
| 9 | 139399507 | C | A | NOTCH1 | p.D1546Y | 0 | 69 | 0.507246 | 69 | WCM243_X1_1 | Tissue |
| 9 | 34256113 | G | T | KIF24 | p.H1164Q | 0 | 57 | 0.074627 | 67 | WCM243_X1_1 | Tissue |
| 12 | 117612522 | C | T | FBXO21 | p.V225M | 0 | 34 | 0.1125 | 80 | WCM247 | Plasma |
| 5 | 140202639 | C | T | PCDHA5 | p.R427W | 0 | 31 | 0.092593 | 108 | WCM247 | Plasma |
| 8 | 56436599 | G | A | XKR4 | p.R589Q | 0 | 37 | 0.065934 | 91 | WCM247 | Plasma |
| 10 | 105836152 | A | T | COL17A1 | p.Y80N | 0 | 111 | 0.197368 | 76 | WCM247_Z9_1 | Tissue |
| 11 | 17099184 | C | G | RPS13 | p.G2A | 0 | 146 | 0.108527 | 129 | WCM247_Z9_1 | Tissue |
| 1 | 157512685 | C | T | FCRL5 | p.A363T | 0 | 151 | 0.184049 | 163 | WCM247_Z9_1 | Tissue |
| 11 | 78433758 | C | A | TENM4 | p.R1252I | 0 | 85 | 0.105691 | 123 | WCM247_Z9_1 | Tissue |
| 1 | 183221781 | C | T | NMNAT2 | p.G307S | 0 | 93 | 0.245283 | 106 | WCM247_Z9_1 | Tissue |
| 12 | 117612522 | C | T | FBXO21 | p.V225M | 0 | 177 | 0.278607 | 201 | WCM247_Z9_1 | Tissue |
| 12 | 29908650 | T | G | TMTC1 | p.Q241H | 0 | 63 | 0.135135 | 37 | WCM247_Z9_1 | Tissue |
| 1 | 44071048 | C | T | PTPRF | p.S1108F | 0 | 99 | 0.275 | 120 | WCM247_Z9_1 | Tissue |
| 15 | 84237376 | G | T | SH3GL3 | p.D95Y | 0 | 130 | 0.080645 | 62 | WCM247_Z9_1 | Tissue |
| 16 | 84922899 | G | A | CRISPLD2 | p.V457M | 0 | 83 | 0.125 | 104 | WCM247_Z9_1 | Tissue |
| 16 | 89977048 | C | G | TCF25 | p.L617V | 0 | 77 | 0.133333 | 60 | WCM247_Z9_1 | Tissue |
| 17 | 57287675 | C | G | SMG8 | p.P88R | 0 | 86 | 0.244898 | 49 | WCM247_Z9_1 | Tissue |
| 17 | 7577580 | T | C | TP53 | p.Y234C | 0 | 204 | 0.243421 | 152 | WCM247_Z9_1 | Tissue |
| 1 | 93683398 | A | T | CCDC18 | p.H644L | 0 | 45 | 0.088889 | 45 | WCM247_Z9_1 | Tissue |
| 19 | 46998691 | C | T | PNMAL2 | p.R11Q | 0 | 90 | 0.089552 | 67 | WCM247_Z9_1 | Tissue |
| 2 | 101885703 | T | A | CNOT11 | p.V454E | 0 | 31 | 0.228571 | 35 | WCM247_Z9_1 | Tissue |
| 2 | 23865523 | G | A | KLHL29 | p.G248E | 0 | 35 | 0.571429 | 42 | WCM247_Z9_1 | Tissue |
| 3 | 123014966 | A | G | ADCY5 | p.S1010P | 0 | 91 | 0.162162 | 74 | WCM247_Z9_1 | Tissue |
| 3 | 43074492 | C | T | FAM198A | p.T246M | 0 | 120 | 0.282609 | 92 | WCM247_Z9_1 | Tissue |
| 3 | 47044007 | C | G | NBEAL2 | p.F1766L | 0 | 46 | 0.209302 | 43 | WCM247_Z9_1 | Tissue |
| 3 | 63981669 | C | T | ATXN7 | p.S724F | 0 | 95 | 0.254545 | 110 | WCM247_Z9_1 | Tissue |
| 3 | 9798237 | G | A | OGG1 | p.R277H | 0 | 212 | 0.18408 | 201 | WCM247_Z9_1 | Tissue |
| 5 | 140202639 | C | T | PCDHA5 | p.R427W | 0 | 126 | 0.19685 | 127 | WCM247_Z9_1 | Tissue |
| 7 | 86415634 | G | A | GRM3 | p.A176T | 0 | 91 | 0.335821 | 134 | WCM247_Z9_1 | Tissue |
| 8 | 56015436 | G | T | XKR4 | p.V130F | 0 | 143 | 0.189781 | 137 | WCM247_Z9_1 | Tissue |
| 8 | 56436599 | G | A | XKR4 | p.R589Q | 0 | 75 | 0.164557 | 79 | WCM247_Z9_1 | Tissue |
| 8 | 97978169 | C | G | CPQ | p.L286V | 0 | 34 | 0.09901 | 101 | WCM247_Z9_1 | Tissue |
| 1 | 185894321 | G | T | HMCN1 | p.L476F | 0 | 51 | 0.449275 | 69 | WCM252 | Plasma |
| 17 | 4851661 | T | C | PFN1 | p.N10S | 0 | 50 | 0.368421 | 76 | WCM252 | Plasma |
| 17 | 62496142 | G | C | DDX5 | p.P582A | 0 | 35 | 0.054054 | 74 | WCM252 | Plasma |
| 2 | 231134273 | A | C | SP140 | p.M423L | 0 | 51 | 0.346535 | 101 | WCM252 | Plasma |
| 5 | 61785151 | G | A | IPO11 | p.M444I | 0 | 45 | 0.474576 | 59 | WCM252 | Plasma |
| 9 | 96859671 | C | T | PTPDC1 | p.R221W | 0 | 45 | 0.421053 | 114 | WCM252 | Plasma |
| 1 | 110768678 | C | T | KCNC4 | p.P566L | 0 | 69 | 0.3 | 80 | WCM252_Z3_1 | Tissue |
| 11 | 56086504 | G | A | OR8K3 | p.G241E | 0 | 46 | 0.088235 | 68 | WCM252_Z3_1 | Tissue |
| 11 | 6624716 | T | A | RRP8 | p.E6V | 0 | 94 | 0.102362 | 127 | WCM252_Z3_1 | Tissue |
| 1 | 185894321 | G | T | HMCN1 | p.L476F | 0 | 105 | 0.307692 | 52 | WCM252_Z3_1 | Tissue |
| 12 | 121712178 | G | A | CAMKK2 | p.S51F | 0 | 77 | 0.121495 | 107 | WCM252_Z3_1 | Tissue |
| 12 | 124904531 | C | T | NCOR2 | p.R485Q | 0 | 189 | 0.111607 | 224 | WCM252_Z3_1 | Tissue |
| 1 | 245019798 | C | T | HNRNPU | p.G625R | 0 | 39 | 0.083333 | 48 | WCM252_Z3_1 | Tissue |
| 12 | 7842988 | G | A | GDF3 | p.P194L | 0 | 64 | 0.052632 | 95 | WCM252_Z3_1 | Tissue |
| 13 | 20763512 | G | A | GJB2 | p.P70L | 0 | 154 | 0.217617 | 193 | WCM252_Z3_1 | Tissue |
| 13 | 58207821 | T | A | PCDH17 | p.S381T | 0 | 130 | 0.282353 | 85 | WCM252_Z3_1 | Tissue |
| 13 | 58207822 | C | A | PCDH17 | p.S381Y | 0 | 129 | 0.282353 | 85 | WCM252_Z3_1 | Tissue |
| 14 | 91804910 | C | T | CCDC88C | p.D272N | 0 | 101 | 0.107843 | 102 | WCM252_Z3_1 | Tissue |
| 15 | 39882019 | G | A | THBS1 | p.R647H | 0 | 55 | 0.36 | 75 | WCM252_Z3_1 | Tissue |
| 15 | 43500848 | T | A | EPB42 | p.R320* | 0 | 103 | 0.103093 | 97 | WCM252_Z3_1 | Tissue |
| 15 | 69553448 | G | A | GLCE | p.W203* | 0 | 42 | 0.128205 | 39 | WCM252_Z3_1 | Tissue |
| 16 | 23079683 | G | T | USP31 | p.A1248D | 0 | 76 | 0.060606 | 99 | WCM252_Z3_1 | Tissue |
| 16 | 30100319 | G | T | TBX6 | p.P189H | 0 | 72 | 0.134146 | 82 | WCM252_Z3_1 | Tissue |
| 17 | 48639044 | T | A | CACNA1G | p.L75H | 0 | 149 | 0.181818 | 143 | WCM252_Z3_1 | Tissue |
| 17 | 79477798 | A | T | ACTG1 | p.L349Q | 0 | 71 | 0.055556 | 72 | WCM252_Z3_1 | Tissue |
| 17 | 80678231 | G | A | FN3KRP | p.G126D | 0 | 164 | 0.233161 | 193 | WCM252_Z3_1 | Tissue |
| 1 | 978666 | A | G | AGRN | p.T478A | 0 | 86 | 0.150538 | 93 | WCM252_Z3_1 | Tissue |
| 19 | 9087822 | C | A | MUC16 | p.K1331N | 0 | 56 | 0.056818 | 88 | WCM252_Z3_1 | Tissue |
| 2 | 231134273 | A | C | SP140 | p.M423L | 0 | 66 | 0.430556 | 72 | WCM252_Z3_1 | Tissue |
| 3 | 141884523 | T | A | GK5 | p.S511C | 0 | 44 | 0.121212 | 66 | WCM252_Z3_1 | Tissue |
| 4 | 122103913 | A | T | TNIP3 | p.L43Q | 0 | 106 | 0.052632 | 114 | WCM252_Z3_1 | Tissue |
| 4 | 17609154 | G | A | LAP3 | p.G501E | 0 | 65 | 0.1875 | 48 | WCM252_Z3_1 | Tissue |
| 4 | 183676240 | C | A | TENM3 | p.P1574T | 0 | 87 | 0.421875 | 64 | WCM252_Z3_1 | Tissue |
| 4 | 47605450 | A | T | CORIN | p.C926S | 0 | 132 | 0.125 | 136 | WCM252_Z3_1 | Tissue |
| 6 | 167343198 | C | T | RNASET2 | p.G217R | 0 | 218 | 0.17757 | 107 | WCM252_Z3_1 | Tissue |
| 8 | 122641438 | A | T | HAS2 | p.L48Q | 0 | 133 | 0.130769 | 130 | WCM252_Z3_1 | Tissue |
| 8 | 144803964 | C | T | MAPK15 | p.Q458* | 0 | 83 | 0.096774 | 62 | WCM252_Z3_1 | Tissue |
| 8 | 35584026 | C | T | UNC5D | p.R554C | 0 | 154 | 0.316239 | 117 | WCM252_Z3_1 | Tissue |
| 9 | 123783877 | C | A | C5 | p.L404F | 0 | 168 | 0.090909 | 187 | WCM252_Z3_1 | Tissue |
| 9 | 126783486 | G | A | LHX2 | p.R279Q | 0 | 124 | 0.050562 | 178 | WCM252_Z3_1 | Tissue |
| 9 | 96859671 | C | T | PTPDC1 | p.R221W | 0.007407 | 135 | 0.319672 | 122 | WCM252_Z3_1 | Tissue |
| X | 8767075 | C | T | FAM9A | p.R51H | 0 | 42 | 0.541667 | 48 | WCM252_Z3_1 | Tissue |
| X | 96213115 | G | C | DIAPH2 | p.E635Q | 0 | 35 | 0.21875 | 32 | WCM252_Z3_1 | Tissue |
| 11 | 118100616 | G | T | MPZL3 | p.T234K | 0 | 64 | 0.083333 | 36 | WCM253_Z1_1 | Tissue |
| 11 | 14907394 | G | A | CYP2R1 | p.L99F | 0 | 74 | 0.241379 | 87 | WCM253_Z1_1 | Tissue |
| 1 | 1153950 | T | C | SDF4 | p.E267G | 0 | 226 | 0.259804 | 204 | WCM253_Z1_1 | Tissue |
| 1 | 12252505 | G | A | TNFRSF1B | p.V159M | 0 | 528 | 0.183007 | 459 | WCM253_Z1_1 | Tissue |
| 1 | 151680357 | G | T | CELF3 | p.L181I | 0 | 138 | 0.415385 | 195 | WCM253_Z1_1 | Tissue |
| 1 | 151687150 | A | G | CELF3 | p.C50R | 0 | 44 | 0.258065 | 31 | WCM253_Z1_1 | Tissue |
| 1 | 155912410 | C | T | RXFP4 | p.L304F | 0 | 75 | 0.085714 | 70 | WCM253_Z1_1 | Tissue |
| 1 | 158584086 | C | A | SPTA1 | p.G2267C | 0 | 61 | 0.178571 | 56 | WCM253_Z1_1 | Tissue |
| 1 | 158615007 | T | A | SPTA1 | p.K1389* | 0 | 108 | 0.132653 | 98 | WCM253_Z1_1 | Tissue |
| 11 | 58979775 | C | A | MPEG1 | p.R188S | 0 | 79 | 0.089286 | 56 | WCM253_Z1_1 | Tissue |
| 1 | 160144484 | A | G | ATP1A4 | p.D753G | 0 | 85 | 0.103093 | 97 | WCM253_Z1_1 | Tissue |
| 11 | 64535244 | C | T | SF1 | p.G381S | 0 | 125 | 0.069767 | 86 | WCM253_Z1_1 | Tissue |
| 1 | 175360506 | C | G | TNR | p.K475N | 0 | 84 | 0.227723 | 101 | WCM253_Z1_1 | Tissue |
| 1 | 186863272 | G | A | PLA2G4A | p.G103R | 0 | 68 | 0.518182 | 110 | WCM253_Z1_1 | Tissue |
| 1 | 192153607 | A | G | RGS18 | p.N211D | 0 | 67 | 0.275 | 80 | WCM253_Z1_1 | Tissue |
| 12 | 21015752 | G | C | SLCO1B3 | p.A231P | 0 | 144 | 0.123188 | 138 | WCM253_Z1_1 | Tissue |
| 1 | 230493041 | C | T | PGBD5 | p.A51T | 0 | 115 | 0.414894 | 94 | WCM253_Z1_1 | Tissue |
| 12 | 48387269 | G | C | COL2A1 | p.P314R | 0 | 53 | 0.290909 | 55 | WCM253_Z1_1 | Tissue |
| 12 | 64505618 | G | A | SRGAP1 | p.V666I | 0 | 69 | 0.188406 | 69 | WCM253_Z1_1 | Tissue |
| 1 | 27432553 | G | C | SLC9A1 | p.I436M | 0 | 368 | 0.19863 | 292 | WCM253_Z1_1 | Tissue |
| 12 | 95926840 | G | A | USP44 | p.A398V | 0 | 142 | 0.705479 | 146 | WCM253_Z1_1 | Tissue |
| 14 | 23103448 | G | T | OR6J1 | p.T90N | 0 | 84 | 0.230769 | 78 | WCM253_Z1_1 | Tissue |
| 14 | 31872059 | G | C | HEATR5A | p.S41R | 0 | 58 | 0.142857 | 56 | WCM253_Z1_1 | Tissue |
| 14 | 52520480 | C | T | NID2 | p.E416K | 0 | 180 | 0.617647 | 238 | WCM253_Z1_1 | Tissue |
| 14 | 94404173 | C | T | ASB2 | p.E500K | 0 | 50 | 0.311111 | 90 | WCM253_Z1_1 | Tissue |
| 15 | 37390178 | C | T | MEIS2 | p.A79T | 0 | 92 | 0.103448 | 58 | WCM253_Z1_1 | Tissue |
| 16 | 22337107 | C | G | POLR3E | p.D458E | 0 | 90 | 0.28125 | 64 | WCM253_Z1_1 | Tissue |
| 16 | 27689194 | G | A | KIAA0556 | p.D229N | 0 | 87 | 0.157025 | 121 | WCM253_Z1_1 | Tissue |
| 16 | 27689230 | G | C | KIAA0556 | p.D241H | 0 | 95 | 0.164062 | 128 | WCM253_Z1_1 | Tissue |
| 16 | 51174296 | C | T | SALL1 | p.E613K | 0 | 98 | 0.089888 | 89 | WCM253_Z1_1 | Tissue |
| 1 | 67263810 | C | T | INSL5 | p.M98I | 0 | 98 | 0.493671 | 79 | WCM253_Z1_1 | Tissue |
| 17 | 10535189 | T | G | MYH3 | p.K1701Q | 0 | 84 | 0.648649 | 37 | WCM253_Z1_1 | Tissue |
| 17 | 15207287 | T | A | TEKT3 | p.Y480F | 0 | 68 | 0.306667 | 75 | WCM253_Z1_1 | Tissue |
| 17 | 4720324 | T | C | PLD2 | p.F559L | 0 | 49 | 0.432432 | 37 | WCM253_Z1_1 | Tissue |
| 17 | 47395002 | C | T | ZNF652 | p.R29H | 0 | 91 | 0.083333 | 72 | WCM253_Z1_1 | Tissue |
| 18 | 10791250 | C | A | PIEZO2 | p.E611* | 0 | 95 | 0.067568 | 74 | WCM253_Z1_1 | Tissue |
| 1 | 881862 | C | G | NOC2L | p.G575R | 0 | 85 | 0.228571 | 35 | WCM253_Z1_1 | Tissue |
| 19 | 2403107 | G | A | TMPRSS9 | p.C161Y | 0 | 196 | 0.266667 | 135 | WCM253_Z1_1 | Tissue |
| 19 | 2403108 | C | A | TMPRSS9 | p.C161* | 0 | 196 | 0.268657 | 134 | WCM253_Z1_1 | Tissue |
| 19 | 36293972 | C | T | PRODH2 | p.R413H | 0 | 138 | 0.427273 | 110 | WCM253_Z1_1 | Tissue |
| 19 | 8656706 | C | G | ADAMTS10 | p.R592P | 0 | 128 | 0.108696 | 46 | WCM253_Z1_1 | Tissue |
| 20 | 40050229 | A | C | CHD6 | p.F1682L | 0 | 115 | 0.152542 | 118 | WCM253_Z1_1 | Tissue |
| 21 | 47863774 | C | A | PCNT | p.P3251Q | 0 | 71 | 0.070588 | 85 | WCM253_Z1_1 | Tissue |
| 2 | 201846030 | G | T | FAM126B | p.S519Y | 0 | 111 | 0.162791 | 129 | WCM253_Z1_1 | Tissue |
| 2 | 239049727 | C | A | KLHL30 | p.A111E | 0 | 53 | 0.079365 | 63 | WCM253_Z1_1 | Tissue |
| 2 | 32704553 | G | C | BIRC6 | p.A2426P | 0 | 61 | 0.122807 | 57 | WCM253_Z1_1 | Tissue |
| 3 | 12953144 | G | C | IQSEC1 | p.L815V | 0 | 133 | 0.166667 | 84 | WCM253_Z1_1 | Tissue |
| 3 | 167023464 | G | T | ZBBX | p.S564R | 0 | 35 | 0.210526 | 38 | WCM253_Z1_1 | Tissue |
| 3 | 39230124 | C | A | XIRP1 | p.W271C | 0 | 82 | 0.485294 | 68 | WCM253_Z1_1 | Tissue |
| 3 | 69058976 | C | T | EOGT | p.G8R | 0 | 91 | 0.294118 | 34 | WCM253_Z1_1 | Tissue |
| 4 | 110222996 | C | A | COL25A1 | p.Q60H | 0 | 217 | 0.27907 | 129 | WCM253_Z1_1 | Tissue |
| 4 | 126412490 | C | A | FAT4 | p.S4838Y | 0 | 145 | 0.37931 | 58 | WCM253_Z1_1 | Tissue |
| 5 | 26915751 | T | A | CDH9 | p.E170D | 0 | 42 | 0.106383 | 47 | WCM253_Z1_1 | Tissue |
| 5 | 5191866 | G | C | ADAMTS16 | p.G426R | 0 | 162 | 0.109827 | 173 | WCM253_Z1_1 | Tissue |
| 6 | 28474114 | T | C | GPX6 | p.N112D | 0 | 284 | 0.325243 | 206 | WCM253_Z1_1 | Tissue |
| 6 | 39046906 | G | T | GLP1R | p.V325F | 0 | 166 | 0.19337 | 181 | WCM253_Z1_1 | Tissue |
| 6 | 75833695 | C | G | COL12A1 | p.E2280D | 0 | 136 | 0.086207 | 58 | WCM253_Z1_1 | Tissue |
| 7 | 104758428 | A | C | SRPK2 | p.F653V | 0 | 225 | 0.403292 | 243 | WCM253_Z1_1 | Tissue |
| 8 | 28385331 | A | T | FZD3 | p.I352F | 0 | 174 | 0.521277 | 94 | WCM253_Z1_1 | Tissue |
| 8 | 41543767 | G | T | ANK1 | p.D1431E | 0 | 220 | 0.438095 | 105 | WCM253_Z1_1 | Tissue |
| 9 | 34514691 | C | T | DNAI1 | p.A591V | 0 | 117 | 0.355556 | 90 | WCM253_Z1_1 | Tissue |
| 10 | 120883013 | A | G | FAM45A | p.D209G | 0 | 135 | 0.246753 | 77 | WCM261_Z4_1 | Tissue |
| 10 | 99473795 | C | A | MARVELD1 | p.Y54* | 0 | 45 | 0.134615 | 52 | WCM261_Z4_1 | Tissue |
| 11 | 120099678 | C | T | OAF | p.R217C | 0 | 137 | 0.303191 | 188 | WCM261_Z4_1 | Tissue |
| 11 | 62415862 | C | G | INTS5 | p.G564R | 0 | 122 | 0.216 | 125 | WCM261_Z4_1 | Tissue |
| 11 | 6661065 | C | T | DCHS1 | p.G594R | 0 | 277 | 0.158103 | 253 | WCM261_Z4_1 | Tissue |
| 12 | 108939078 | T | C | SART3 | p.Y189C | 0 | 114 | 0.20339 | 118 | WCM261_Z4_1 | Tissue |
| 12 | 123738288 | C | A | C12orf65 | p.L23I | 0 | 122 | 0.229885 | 87 | WCM261_Z4_1 | Tissue |
| 12 | 13243577 | G | A | GSG1 | p.P75L | 0 | 75 | 0.173913 | 69 | WCM261_Z4_1 | Tissue |
| 12 | 22622651 | G | A | C2CD5 | p.P842L | 0 | 66 | 0.340426 | 47 | WCM261_Z4_1 | Tissue |
| 1 | 223939730 | G | A | CAPN2 | p.E311K | 0 | 81 | 0.328244 | 131 | WCM261_Z4_1 | Tissue |
| 12 | 5724394 | T | C | ANO2 | p.N630S | 0 | 84 | 0.17094 | 117 | WCM261_Z4_1 | Tissue |
| 12 | 58006878 | G | T | ARHGEF25 | p.C88F | 0 | 36 | 0.25 | 36 | WCM261_Z4_1 | Tissue |
| 14 | 105945464 | G | T | CRIP2 | p.E137* | 0 | 74 | 0.15873 | 63 | WCM261_Z4_1 | Tissue |
| 14 | 94254001 | C | T | PRIMA1 | p.A22T | 0 | 79 | 0.175 | 80 | WCM261_Z4_1 | Tissue |
| 1 | 55223781 | T | C | PARS2 | p.I352V | 0 | 66 | 0.222222 | 54 | WCM261_Z4_1 | Tissue |
| 15 | 89857911 | T | A | FANCI | p.F1263L | 0 | 155 | 0.156716 | 134 | WCM261_Z4_1 | Tissue |
| 15 | 89857912 | C | T | FANCI | p.L1264F | 0 | 156 | 0.156716 | 134 | WCM261_Z4_1 | Tissue |
| 16 | 1995589 | C | T | RPL3L | p.V360M | 0 | 171 | 0.24812 | 133 | WCM261_Z4_1 | Tissue |
| 16 | 816887 | C | T | MSLN | p.A467V | 0 | 54 | 0.339286 | 56 | WCM261_Z4_1 | Tissue |
| 17 | 47696450 | A | C | SPOP | p.F125V | 0 | 113 | 0.383562 | 73 | WCM261_Z4_1 | Tissue |
| 17 | 61790789 | T | A | STRADA | p.N109Y | 0 | 101 | 0.157895 | 95 | WCM261_Z4_1 | Tissue |
| 17 | 79477798 | A | T | ACTG1 | p.L349Q | 0 | 77 | 0.117647 | 68 | WCM261_Z4_1 | Tissue |
| 17 | 8198082 | A | G | SLC25A35 | p.V15A | 0 | 70 | 0.225806 | 62 | WCM261_Z4_1 | Tissue |
| 19 | 49316453 | G | T | HSD17B14 | p.D264E | 0 | 153 | 0.101695 | 177 | WCM261_Z4_1 | Tissue |
| 20 | 30720912 | G | A | TM9SF4 | p.D38N | 0 | 50 | 0.074074 | 54 | WCM261_Z4_1 | Tissue |
| 2 | 182423323 | C | T | CERKL | p.A290T | 0 | 35 | 0.414634 | 41 | WCM261_Z4_1 | Tissue |
| 2 | 88407999 | G | T | SMYD1 | p.A419S | 0 | 123 | 0.189189 | 111 | WCM261_Z4_1 | Tissue |
| 3 | 155632310 | C | G | GMPS | p.T330S | 0 | 81 | 0.064935 | 77 | WCM261_Z4_1 | Tissue |
| 3 | 170108053 | A | C | SKIL | p.E491A | 0 | 63 | 0.515625 | 64 | WCM261_Z4_1 | Tissue |
| 3 | 38991699 | G | C | SCN11A | p.P52R | 0 | 69 | 0.105263 | 57 | WCM261_Z4_1 | Tissue |
| 3 | 39374616 | G | A | CCR8 | p.S265N | 0 | 81 | 0.156627 | 83 | WCM261_Z4_1 | Tissue |
| 3 | 44489502 | G | A | ZNF445 | p.S554L | 0 | 95 | 0.290698 | 86 | WCM261_Z4_1 | Tissue |
| 4 | 57237798 | T | C | AASDH | p.D227G | 0 | 60 | 0.096774 | 31 | WCM261_Z4_1 | Tissue |
| 6 | 152708456 | C | A | SYNE1 | p.E2746D | 0 | 55 | 0.075 | 40 | WCM261_Z4_1 | Tissue |
| 6 | 24547442 | C | A | KIAA0319 | p.G1057V | 0 | 47 | 0.297297 | 37 | WCM261_Z4_1 | Tissue |
| 6 | 41895258 | A | C | BYSL | p.K139Q | 0 | 265 | 0.183746 | 283 | WCM261_Z4_1 | Tissue |
| 6 | 65327345 | G | T | EYS | p.P1217H | 0 | 90 | 0.152174 | 92 | WCM261_Z4_1 | Tissue |
| 7 | 130364004 | G | A | TSGA13 | p.L126F | 0 | 68 | 0.191176 | 68 | WCM261_Z4_1 | Tissue |
| 8 | 124749564 | C | T | ANXA13 | p.R4H | 0 | 41 | 0.12766 | 47 | WCM261_Z4_1 | Tissue |
| 9 | 108147760 | G | C | SLC44A1 | p.D643H | 0 | 52 | 0.157895 | 38 | WCM261_Z4_1 | Tissue |
| 9 | 4793179 | G | A | RCL1 | p.V30I | 0 | 63 | 0.26 | 50 | WCM261_Z4_1 | Tissue |
| 9 | 5906943 | G | A | MLANA | p.G78E | 0 | 56 | 0.356164 | 73 | WCM261_Z4_1 | Tissue |
| 9 | 73230925 | G | C | TRPM3 | p.P797A | 0 | 148 | 0.210084 | 119 | WCM261_Z4_1 | Tissue |
| 9 | 95077237 | C | T | NOL8 | p.G557D | 0 | 111 | 0.149425 | 87 | WCM261_Z4_1 | Tissue |
| X | 152821522 | C | T | ATP2B3 | p.R692* | 0 | 145 | 0.535088 | 114 | WCM261_Z4_1 | Tissue |
| X | 2772191 | C | T | GYG2 | p.T138I | 0 | 118 | 0.405063 | 79 | WCM261_Z4_1 | Tissue |
| 10 | 64219525 | G | A | ZNF365 | p.R317H | 0 | 107 | 0.123596 | 89 | WCM2_Z3_1 | Tissue |
| 11 | 57518560 | C | T | BTBD18 | p.C34Y | 0 | 112 | 0.213592 | 103 | WCM2_Z3_1 | Tissue |
| 11 | 67925529 | G | C | SUV420H1 | p.L762V | 0 | 54 | 0.333333 | 33 | WCM2_Z3_1 | Tissue |
| 12 | 56365029 | T | C | CDK2 | p.S264P | 0 | 139 | 0.228395 | 162 | WCM2_Z3_1 | Tissue |
| 12 | 57598451 | A | C | LRP1 | p.N3705H | 0 | 302 | 0.301887 | 212 | WCM2_Z3_1 | Tissue |
| 13 | 114507910 | A | G | TMEM255B | p.Y241C | 0 | 53 | 0.304348 | 46 | WCM2_Z3_1 | Tissue |
| 1 | 36886153 | C | T | OSCP1 | p.G305R | 0 | 186 | 0.163121 | 141 | WCM2_Z3_1 | Tissue |
| 14 | 105246526 | C | A | AKT1 | p.R25L | 0 | 126 | 0.155844 | 77 | WCM2_Z3_1 | Tissue |
| 15 | 77236202 | A | C | RCN2 | p.D184A | 0 | 60 | 0.25 | 32 | WCM2_Z3_1 | Tissue |
| 15 | 90193016 | G | C | KIF7 | p.A162G | 0 | 55 | 0.121951 | 41 | WCM2_Z3_1 | Tissue |
| 1 | 6659260 | T | C | KLHL21 | p.Y425C | 0 | 68 | 0.122807 | 57 | WCM2_Z3_1 | Tissue |
| 16 | 706421 | G | T | WDR90 | p.A696S | 0 | 55 | 0.144578 | 83 | WCM2_Z3_1 | Tissue |
| 16 | 72107856 | A | G | HPR | p.N24S | 0 | 230 | 0.216783 | 143 | WCM2_Z3_1 | Tissue |
| 19 | 34857731 | A | G | GPI | p.N86S | 0 | 59 | 0.147541 | 61 | WCM2_Z3_1 | Tissue |
| 1 | 94520757 | C | A | ABCA4 | p.E833* | 0 | 48 | 0.294118 | 34 | WCM2_Z3_1 | Tissue |
| 19 | 45322365 | G | T | BCAM | p.W463C | 0 | 213 | 0.485714 | 70 | WCM2_Z3_1 | Tissue |
| 20 | 32212668 | T | G | CBFA2T2 | p.L273R | 0 | 218 | 0.343042 | 309 | WCM2_Z3_1 | Tissue |
| 20 | 57769643 | G | A | ZNF831 | p.R1190H | 0 | 48 | 0.5 | 56 | WCM2_Z3_1 | Tissue |
| 20 | 62164907 | G | A | PTK6 | p.R223* | 0 | 119 | 0.25 | 112 | WCM2_Z3_1 | Tissue |
| 2 | 15601386 | G | C | NBAS | p.L760V | 0 | 79 | 0.096774 | 62 | WCM2_Z3_1 | Tissue |
| 2 | 220078380 | G | C | ABCB6 | p.D529E | 0 | 293 | 0.141414 | 297 | WCM2_Z3_1 | Tissue |
| 2 | 228883310 | A | T | SPHKAP | p.S754T | 0 | 47 | 0.1875 | 32 | WCM2_Z3_1 | Tissue |
| 3 | 170244539 | C | T | SLC7A14 | p.V63I | 0 | 47 | 0.345455 | 55 | WCM2_Z3_1 | Tissue |
| 3 | 35729235 | C | T | ARPP21 | p.S89L | 0 | 139 | 0.189394 | 132 | WCM2_Z3_1 | Tissue |
| 3 | 49063981 | T | C | IMPDH2 | p.Y294C | 0 | 106 | 0.170543 | 129 | WCM2_Z3_1 | Tissue |
| 3 | 50677812 | C | T | MAPKAPK3 | p.P79S | 0 | 97 | 0.307018 | 114 | WCM2_Z3_1 | Tissue |
| 4 | 71554892 | G | C | UTP3 | p.Q166H | 0 | 144 | 0.25 | 136 | WCM2_Z3_1 | Tissue |
| 5 | 149782709 | T | G | CD74 | p.R264S | 0 | 83 | 0.064516 | 62 | WCM2_Z3_1 | Tissue |
| 5 | 37121743 | C | A | C5orf42 | p.C3000F | 0 | 44 | 0.441176 | 34 | WCM2_Z3_1 | Tissue |
| 6 | 116833031 | C | G | FAM26E | p.P58A | 0 | 76 | 0.211538 | 104 | WCM2_Z3_1 | Tissue |
| 7 | 122342633 | C | G | RNF148 | p.E58Q | 0 | 65 | 0.181818 | 66 | WCM2_Z3_1 | Tissue |
| 7 | 53103860 | G | A | POM121L12 | p.A166T | 0 | 83 | 0.235849 | 106 | WCM2_Z3_1 | Tissue |
| 7 | 5414109 | G | A | TNRC18 | p.R936W | 0 | 238 | 0.263359 | 262 | WCM2_Z3_1 | Tissue |
| 8 | 29989567 | C | G | MBOAT4 | p.R400S | 0 | 341 | 0.215385 | 325 | WCM2_Z3_1 | Tissue |
| 9 | 94495697 | G | A | ROR2 | p.T215M | 0 | 156 | 0.378151 | 119 | WCM2_Z3_1 | Tissue |
| X | 118140114 | C | A | LONRF3 | p.C482* | 0 | 43 | 0.387097 | 31 | WCM2_Z3_1 | Tissue |
| 10 | 64936214 | A | C | JMJD1C | p.L2415R | 0 | 61 | 0.316239 | 117 | WCM341 | Plasma |
| 10 | 89624301 | G | C | PTEN | p.L25F | 0 | 33 | 0.25 | 44 | WCM341 | Plasma |
| 1 | 163042226 | A | C | RGS4 | p.K29T | 0 | 59 | 0.066038 | 106 | WCM341 | Plasma |
| 1 | 203194906 | G | T | CHIT1 | p.L50I | 0 | 43 | 0.05102 | 98 | WCM341 | Plasma |
| 1 | 204103659 | C | T | ETNK2 | p.D358N | 0 | 35 | 0.052632 | 95 | WCM341 | Plasma |
| 12 | 22610051 | G | A | C2CD5 | p.R860W | 0 | 69 | 0.252252 | 111 | WCM341 | Plasma |
| 1 | 24409176 | T | C | MYOM3 | p.K667E | 0 | 44 | 0.218391 | 87 | WCM341 | Plasma |
| 14 | 23893217 | G | A | MYH7 | p.R941C | 0 | 39 | 0.201835 | 109 | WCM341 | Plasma |
| 14 | 94754861 | C | T | SERPINA10 | p.E252K | 0 | 40 | 0.325301 | 83 | WCM341 | Plasma |
| 17 | 48917416 | G | A | WFIKKN2 | p.R256Q | 0 | 53 | 0.371795 | 78 | WCM341 | Plasma |
| 19 | 15794478 | C | T | CYP4F12 | p.R275C | 0 | 47 | 0.195402 | 87 | WCM341 | Plasma |
| 19 | 39660270 | A | G | PAK4 | p.D26G | 0 | 59 | 0.081395 | 86 | WCM341 | Plasma |
| 19 | 42738770 | G | A | GSK3A | p.R243C | 0 | 34 | 0.277778 | 54 | WCM341 | Plasma |
| 19 | 49387247 | C | A | TULP2 | p.R373L | 0 | 48 | 0.066038 | 106 | WCM341 | Plasma |
| 19 | 50750329 | C | T | MYH14 | p.R419C | 0 | 38 | 0.080808 | 99 | WCM341 | Plasma |
| 20 | 43753013 | C | T | WFDC12 | p.E26K | 0 | 35 | 0.411765 | 85 | WCM341 | Plasma |
| 2 | 100900812 | C | T | LONRF2 | p.R738H | 0 | 34 | 0.136986 | 73 | WCM341 | Plasma |
| 2 | 153526876 | C | T | PRPF40A | p.R500Q | 0 | 53 | 0.084337 | 83 | WCM341 | Plasma |
| 2 | 1677426 | A | C | PXDN | p.F336C | 0 | 39 | 0.318182 | 88 | WCM341 | Plasma |
| 2 | 192863795 | G | A | TMEFF2 | p.R226* | 0 | 61 | 0.095238 | 84 | WCM341 | Plasma |
| 2 | 216259383 | C | A | FN1 | p.E1222* | 0 | 75 | 0.052632 | 114 | WCM341 | Plasma |
| 22 | 42992268 | C | T | POLDIP3 | p.R246Q | 0 | 48 | 0.188034 | 117 | WCM341 | Plasma |
| 2 | 37468845 | G | A | NDUFAF7 | p.R178Q | 0 | 77 | 0.359375 | 128 | WCM341 | Plasma |
| 3 | 52430694 | C | T | DNAH1 | p.R3831C | 0 | 32 | 0.065789 | 76 | WCM341 | Plasma |
| 4 | 1341974 | G | A | UVSSA | p.C32Y | 0 | 44 | 0.089286 | 56 | WCM341 | Plasma |
| 4 | 46314718 | C | T | GABRA2 | p.V91I | 0 | 80 | 0.189189 | 111 | WCM341 | Plasma |
| 5 | 9337840 | A | C | SEMA5A | p.L70R | 0 | 39 | 0.283582 | 67 | WCM341 | Plasma |
| 6 | 157150520 | G | A | ARID1B | p.G568R | 0 | 40 | 0.422222 | 90 | WCM341 | Plasma |
| 6 | 159653153 | T | C | FNDC1 | p.S537P | 0 | 43 | 0.443038 | 79 | WCM341 | Plasma |
| 7 | 141952420 | C | T | PRSS58 | p.D150N | 0 | 57 | 0.072072 | 111 | WCM341 | Plasma |
| 8 | 28420374 | G | A | FZD3 | p.R616Q | 0 | 41 | 0.384615 | 104 | WCM341 | Plasma |
| 9 | 32436385 | C | T | ACO1 | p.S746F | 0 | 43 | 0.051546 | 97 | WCM341 | Plasma |
| 9 | 80336342 | G | A | GNAQ | p.S326F | 0 | 41 | 0.087379 | 103 | WCM341 | Plasma |
| 11 | 2798256 | G | A | KCNQ1 | p.V576I | 0 | 213 | 0.061818 | 275 | WCM341_X1_1_p2 | Tissue |
| 11 | 66837995 | C | T | RHOD | p.R144* | 0 | 54 | 0.08 | 75 | WCM341_X1_1_p2 | Tissue |
| 12 | 22610051 | G | A | C2CD5 | p.R860W | 0 | 151 | 0.091892 | 185 | WCM341_X1_1_p2 | Tissue |
| 1 | 24409176 | T | C | MYOM3 | p.K667E | 0 | 176 | 0.081545 | 233 | WCM341_X1_1_p2 | Tissue |
| 12 | 56398237 | A | G | SUOX | p.Q355R | 0 | 66 | 0.235294 | 68 | WCM341_X1_1_p2 | Tissue |
| 13 | 26144947 | G | A | ATP8A2 | p.V506M | 0.009174 | 109 | 0.181818 | 99 | WCM341_X1_1_p2 | Tissue |
| 1 | 34498248 | C | T | CSMD2 | p.R155H | 0 | 63 | 0.196721 | 61 | WCM341_X1_1_p2 | Tissue |
| 13 | 61986379 | G | A | PCDH20 | p.P618L | 0 | 50 | 0.140845 | 71 | WCM341_X1_1_p2 | Tissue |
| 14 | 58831848 | A | C | ARID4A | p.D1014A | 0 | 95 | 0.054795 | 146 | WCM341_X1_1_p2 | Tissue |
| 14 | 94754861 | C | T | SERPINA10 | p.E252K | 0.004032 | 248 | 0.231788 | 151 | WCM341_X1_1_p2 | Tissue |
| 15 | 33954809 | C | T | RYR3 | p.T1693M | 0 | 99 | 0.189189 | 74 | WCM341_X1_1_p2 | Tissue |
| 17 | 37331720 | C | T | CACNB1 | p.R508Q | 0 | 122 | 0.193548 | 124 | WCM341_X1_1_p2 | Tissue |
| 17 | 48917416 | G | A | WFIKKN2 | p.R256Q | 0 | 104 | 0.2 | 80 | WCM341_X1_1_p2 | Tissue |
| 19 | 15794478 | C | T | CYP4F12 | p.R275C | 0 | 82 | 0.152542 | 59 | WCM341_X1_1_p2 | Tissue |
| 19 | 39660270 | A | G | PAK4 | p.D26G | 0 | 51 | 0.087719 | 57 | WCM341_X1_1_p2 | Tissue |
| 19 | 42738770 | G | A | GSK3A | p.R243C | 0 | 168 | 0.144144 | 111 | WCM341_X1_1_p2 | Tissue |
| 19 | 47151848 | G | A | DACT3 | p.A594V | 0 | 32 | 0.081633 | 49 | WCM341_X1_1_p2 | Tissue |
| 19 | 56466262 | C | T | NLRP8 | p.Q280* | 0 | 120 | 0.107692 | 130 | WCM341_X1_1_p2 | Tissue |
| 20 | 18297333 | G | A | ZNF133 | p.R613Q | 0 | 102 | 0.085714 | 70 | WCM341_X1_1_p2 | Tissue |
| 20 | 31023042 | G | T | ASXL1 | p.V843L | 0 | 198 | 0.219178 | 219 | WCM341_X1_1_p2 | Tissue |
| 20 | 57415343 | G | A | GNAS | p.R61H | 0 | 114 | 0.128205 | 117 | WCM341_X1_1_p2 | Tissue |
| 2 | 1677426 | A | C | PXDN | p.F336C | 0 | 104 | 0.296296 | 54 | WCM341_X1_1_p2 | Tissue |
| 22 | 42992268 | C | T | POLDIP3 | p.R246Q | 0 | 153 | 0.117647 | 136 | WCM341_X1_1_p2 | Tissue |
| 2 | 37468845 | G | A | NDUFAF7 | p.R178Q | 0 | 116 | 0.173913 | 69 | WCM341_X1_1_p2 | Tissue |
| 3 | 147113965 | C | T | ZIC4 | p.R121H | 0 | 181 | 0.101351 | 148 | WCM341_X1_1_p2 | Tissue |
| 5 | 140783253 | G | A | PCDHGA9 | p.R245Q | 0 | 80 | 0.058824 | 85 | WCM341_X1_1_p2 | Tissue |
| 5 | 176562856 | C | T | NSD1 | p.A251V | 0 | 36 | 0.073171 | 41 | WCM341_X1_1_p2 | Tissue |
| 6 | 157150520 | G | A | ARID1B | p.G568R | 0 | 204 | 0.256098 | 164 | WCM341_X1_1_p2 | Tissue |
| 8 | 1497134 | C | T | DLGAP2 | p.T92M | 0 | 86 | 0.070175 | 114 | WCM341_X1_1_p2 | Tissue |
| 8 | 28420374 | G | A | FZD3 | p.R616Q | 0 | 46 | 0.3125 | 32 | WCM341_X1_1_p2 | Tissue |
| 9 | 32436385 | C | T | ACO1 | p.S746F | 0 | 39 | 0.078947 | 38 | WCM341_X1_1_p2 | Tissue |
| 1 | 152883120 | C | A | IVL | p.L283M | 0 | 46 | 0.122449 | 49 | WCM341_Z2_1 | Tissue |
| 12 | 22610051 | G | A | C2CD5 | p.R860W | 0 | 151 | 0.264901 | 151 | WCM341_Z2_1 | Tissue |
| 12 | 54367092 | G | A | HOXC11 | p.G23S | 0 | 101 | 0.132653 | 98 | WCM341_Z2_1 | Tissue |
| 12 | 56398237 | A | G | SUOX | p.Q355R | 0 | 66 | 0.364583 | 96 | WCM341_Z2_1 | Tissue |
| 13 | 26144947 | G | A | ATP8A2 | p.V506M | 0.009174 | 109 | 0.505495 | 91 | WCM341_Z2_1 | Tissue |
| 1 | 34498248 | C | T | CSMD2 | p.R155H | 0 | 63 | 0.394366 | 71 | WCM341_Z2_1 | Tissue |
| 13 | 61986379 | G | A | PCDH20 | p.P618L | 0 | 50 | 0.239437 | 71 | WCM341_Z2_1 | Tissue |
| 1 | 44415377 | G | A | IPO13 | p.V125M | 0 | 106 | 0.116883 | 77 | WCM341_Z2_1 | Tissue |
| 14 | 58831848 | A | C | ARID4A | p.D1014A | 0 | 95 | 0.062937 | 143 | WCM341_Z2_1 | Tissue |
| 14 | 94754861 | C | T | SERPINA10 | p.E252K | 0.004032 | 248 | 0.358621 | 145 | WCM341_Z2_1 | Tissue |
| 15 | 33954809 | C | T | RYR3 | p.T1693M | 0 | 99 | 0.221429 | 140 | WCM341_Z2_1 | Tissue |
| 17 | 37331720 | C | T | CACNB1 | p.R508Q | 0 | 122 | 0.42 | 100 | WCM341_Z2_1 | Tissue |
| 17 | 48917416 | G | A | WFIKKN2 | p.R256Q | 0 | 104 | 0.304348 | 69 | WCM341_Z2_1 | Tissue |
| 17 | 80136396 | G | A | CCDC57 | p.A487V | 0 | 111 | 0.140625 | 64 | WCM341_Z2_1 | Tissue |
| 19 | 39660270 | A | G | PAK4 | p.D26G | 0 | 51 | 0.05618 | 89 | WCM341_Z2_1 | Tissue |
| 19 | 42738770 | G | A | GSK3A | p.R243C | 0 | 168 | 0.395349 | 86 | WCM341_Z2_1 | Tissue |
| 19 | 50750329 | C | T | MYH14 | p.R419C | 0 | 143 | 0.104167 | 96 | WCM341_Z2_1 | Tissue |
| 19 | 56466262 | C | T | NLRP8 | p.Q280* | 0 | 120 | 0.142292 | 253 | WCM341_Z2_1 | Tissue |
| 20 | 31023042 | G | T | ASXL1 | p.V843L | 0 | 198 | 0.318182 | 176 | WCM341_Z2_1 | Tissue |
| 20 | 43753013 | C | T | WFDC12 | p.E26K | 0 | 82 | 0.505618 | 89 | WCM341_Z2_1 | Tissue |
| 20 | 57415343 | G | A | GNAS | p.R61H | 0 | 114 | 0.151351 | 185 | WCM341_Z2_1 | Tissue |
| 2 | 100900812 | C | T | LONRF2 | p.R738H | 0 | 37 | 0.177778 | 45 | WCM341_Z2_1 | Tissue |
| 2 | 153526876 | C | T | PRPF40A | p.R500Q | 0 | 112 | 0.141026 | 78 | WCM341_Z2_1 | Tissue |
| 2 | 1677426 | A | C | PXDN | p.F336C | 0 | 104 | 0.423729 | 59 | WCM341_Z2_1 | Tissue |
| 2 | 179732764 | C | T | CCDC141 | p.V855M | 0 | 42 | 0.4 | 55 | WCM341_Z2_1 | Tissue |
| 22 | 21107282 | G | A | PI4KA | p.R908W | 0 | 91 | 0.064103 | 78 | WCM341_Z2_1 | Tissue |
| 22 | 42992268 | C | T | POLDIP3 | p.R246Q | 0 | 153 | 0.2375 | 160 | WCM341_Z2_1 | Tissue |
| 2 | 33752258 | C | T | RASGRP3 | p.R288C | 0 | 76 | 0.126214 | 103 | WCM341_Z2_1 | Tissue |
| 2 | 37468845 | G | A | NDUFAF7 | p.R178Q | 0 | 116 | 0.442857 | 70 | WCM341_Z2_1 | Tissue |
| 2 | 96962311 | C | T | SNRNP200 | p.R545H | 0 | 77 | 0.072727 | 55 | WCM341_Z2_1 | Tissue |
| 3 | 147113965 | C | T | ZIC4 | p.R121H | 0 | 181 | 0.155738 | 122 | WCM341_Z2_1 | Tissue |
| 5 | 109974035 | G | A | TMEM232 | p.A122V | 0 | 58 | 0.066667 | 45 | WCM341_Z2_1 | Tissue |
| 5 | 140783253 | G | A | PCDHGA9 | p.R245Q | 0 | 80 | 0.145833 | 48 | WCM341_Z2_1 | Tissue |
| 5 | 176562856 | C | T | NSD1 | p.A251V | 0 | 36 | 0.090909 | 33 | WCM341_Z2_1 | Tissue |
| 6 | 157150520 | G | A | ARID1B | p.G568R | 0 | 204 | 0.351145 | 131 | WCM341_Z2_1 | Tissue |
| 6 | 52616491 | C | T | GSTA2 | p.G144R | 0 | 75 | 0.117647 | 102 | WCM341_Z2_1 | Tissue |
| 8 | 144946671 | C | T | EPPK1 | p.G251S | 0 | 35 | 0.093023 | 43 | WCM341_Z2_1 | Tissue |
| 9 | 32436385 | C | T | ACO1 | p.S746F | 0 | 39 | 0.075 | 40 | WCM341_Z2_1 | Tissue |
| 11 | 68191056 | G | A | LRP5 | p.E1043K | 0 | 59 | 0.073171 | 123 | WCM365 | Plasma |
| 11 | 95826086 | G | A | MAML2 | p.P370L | 0 | 39 | 0.396825 | 63 | WCM365 | Plasma |
| 12 | 117962698 | C | A | KSR2 | p.M726I | 0 | 49 | 0.231707 | 82 | WCM365 | Plasma |
| 14 | 45696920 | A | T | MIS18BP1 | p.D564E | 0 | 72 | 0.081633 | 98 | WCM365 | Plasma |
| 15 | 65684663 | G | T | IGDCC4 | p.S644Y | 0 | 31 | 0.285714 | 42 | WCM365 | Plasma |
| 16 | 27506176 | T | A | GTF3C1 | p.R896W | 0 | 34 | 0.342857 | 70 | WCM365 | Plasma |
| 17 | 1028687 | G | A | ABR | p.T26M | 0 | 118 | 0.395161 | 124 | WCM365 | Plasma |
| 17 | 39551769 | T | A | KRT31 | p.Y232F | 0 | 45 | 0.089744 | 78 | WCM365 | Plasma |
| 17 | 44249055 | G | C | KANSL1 | p.A152G | 0 | 60 | 0.084507 | 71 | WCM365 | Plasma |
| 21 | 17135247 | C | T | USP25 | p.T28M | 0 | 73 | 0.284091 | 88 | WCM365 | Plasma |
| 2 | 218954730 | C | T | RUFY4 | p.R557C | 0 | 36 | 0.057143 | 70 | WCM365 | Plasma |
| 2 | 219563523 | G | A | STK36 | p.A1086T | 0 | 39 | 0.107143 | 56 | WCM365 | Plasma |
| 2 | 26437359 | G | A | HADHA | p.R291* | 0 | 59 | 0.06 | 100 | WCM365 | Plasma |
| 3 | 127325123 | A | G | MCM2 | p.N279S | 0 | 52 | 0.079365 | 63 | WCM365 | Plasma |
| 3 | 164750342 | G | C | SI | p.H902D | 0 | 72 | 0.060606 | 99 | WCM365 | Plasma |
| 3 | 17053266 | A | T | PLCL2 | p.I684F | 0 | 51 | 0.094595 | 74 | WCM365 | Plasma |
| 4 | 113303691 | G | C | ALPK1 | p.G87R | 0 | 61 | 0.163043 | 92 | WCM365 | Plasma |
| 5 | 110075047 | C | T | SLC25A46 | p.P76L | 0 | 72 | 0.285714 | 49 | WCM365 | Plasma |
| 5 | 133311588 | G | A | VDAC1 | p.Q226* | 0 | 44 | 0.259259 | 54 | WCM365 | Plasma |
| 5 | 224505 | G | C | SDHA | p.E61Q | 0 | 46 | 0.050633 | 79 | WCM365 | Plasma |
| 6 | 118790366 | T | A | CEP85L | p.D708V | 0 | 50 | 0.118421 | 76 | WCM365 | Plasma |
| 6 | 123126094 | C | A | SMPDL3A | p.S260Y | 0 | 66 | 0.066667 | 90 | WCM365 | Plasma |
| 7 | 18705935 | G | A | HDAC9 | p.A520T | 0 | 34 | 0.288889 | 45 | WCM365 | Plasma |
| 9 | 100124651 | A | G | CCDC180 | p.K1255R | 0 | 34 | 0.40625 | 64 | WCM365 | Plasma |
| 9 | 32492500 | T | A | DDX58 | p.K154* | 0 | 76 | 0.516129 | 155 | WCM365 | Plasma |
| 9 | 90500073 | C | A | SPATA31E1 | p.P224Q | 0 | 36 | 0.196429 | 56 | WCM365 | Plasma |
| X | 48211539 | G | T | SSX3 | p.P109Q | 0 | 114 | 0.119497 | 159 | WCM365 | Plasma |
| X | 70444128 | A | G | GJB1 | p.T191A | 0 | 39 | 0.086957 | 69 | WCM365 | Plasma |
| 10 | 48370665 | T | A | ZNF488 | p.C45S | 0 | 186 | 0.096257 | 187 | WCM365_Z2_1 | Tissue |
| 11 | 120996471 | G | T | TECTA | p.C555F | 0 | 103 | 0.131868 | 91 | WCM365_Z2_1 | Tissue |
| 11 | 18231471 | G | A | RP11- | p.R86Q | 0.009259 | 216 | 0.37963 | 216 | WCM365_Z2_1 | Tissue |
| | | | | 113D6.10 | | | | | | | |
| 11 | 1858448 | G | T | SYT8 | p.W331C | 0 | 221 | 0.173203 | 306 | WCM365_Z2_1 | Tissue |
| 11 | 56000214 | C | A | OR5T2 | p.E150* | 0 | 142 | 0.167832 | 143 | WCM365_Z2_1 | Tissue |
| 11 | 95826086 | G | A | MAML2 | p.P370L | 0 | 201 | 0.395745 | 235 | WCM365_Z2_1 | Tissue |
| 12 | 133237686 | C | T | POLE | p.G977R | 0 | 105 | 0.089109 | 101 | WCM365_Z2_1 | Tissue |
| 1 | 39352229 | C | A | RHBDL2 | p.A287S | 0 | 76 | 0.115385 | 52 | WCM365_Z2_1 | Tissue |
| 1 | 43739015 | C | T | TMEM125 | p.R208C | 0 | 263 | 0.489362 | 188 | WCM365_Z2_1 | Tissue |
| 15 | 35212564 | G | C | AQR | p.T397S | 0 | 55 | 0.264706 | 34 | WCM365_Z2_1 | Tissue |
| 16 | 27506176 | T | A | GTF3C1 | p.R896W | 0 | 88 | 0.464912 | 114 | WCM365_Z2_1 | Tissue |
| 17 | 1028687 | G | A | ABR | p.T26M | 0 | 267 | 0.328084 | 381 | WCM365_Z2_1 | Tissue |
| 17 | 43893886 | C | T | CRHR1 | p.A60V | 0 | 122 | 0.111111 | 72 | WCM365_Z2_1 | Tissue |
| 1 | 75037769 | G | A | C1orf173 | p.R1209C | 0 | 321 | 0.424419 | 172 | WCM365_Z2_1 | Tissue |
| 17 | 7578403 | C | A | TP53 | p.C176F | 0 | 171 | 0.410256 | 117 | WCM365_Z2_1 | Tissue |
| 18 | 43481106 | G | C | EPG5 | p.R1501G | 0 | 51 | 0.189189 | 37 | WCM365_Z2_1 | Tissue |
| 19 | 18280021 | G | T | PIK3R2 | p.V702L | 0 | 86 | 0.375 | 80 | WCM365_Z2_1 | Tissue |
| 19 | 3543842 | G | A | C19orf71 | p.R155Q | 0 | 119 | 0.058824 | 85 | WCM365_Z2_1 | Tissue |
| 19 | 9057411 | G | T | MUC16 | p.S10012Y | 0 | 79 | 0.094828 | 116 | WCM365_Z2_1 | Tissue |
| 19 | 9362223 | T | A | OR7E24 | p.F168L | 0 | 74 | 0.166667 | 132 | WCM365_Z2_1 | Tissue |
| 22 | 20229347 | C | T | RTN4R | p.G437R | 0 | 49 | 0.15 | 40 | WCM365_Z2_1 | Tissue |
| 2 | 227896939 | C | T | COL4A4 | p.E1211K | 0 | 189 | 0.243523 | 193 | WCM365_Z2_1 | Tissue |
| 2 | 231313828 | C | A | SP100 | p.S183R | 0 | 51 | 0.057692 | 52 | WCM365_Z2_1 | Tissue |
| 2 | 97217824 | G | C | ARID5A | p.G520A | 0 | 88 | 0.165605 | 157 | WCM365_Z2_1 | Tissue |
| 3 | 38017293 | G | A | CTDSPL | p.V205M | 0 | 189 | 0.209677 | 124 | WCM365_Z2_1 | Tissue |
| 4 | 113303691 | G | C | ALPK1 | p.G87R | 0 | 145 | 0.230769 | 104 | WCM365_Z2_1 | Tissue |
| 4 | 47548757 | C | A | ATP10D | p.H505N | 0 | 299 | 0.167164 | 335 | WCM365_Z2_1 | Tissue |
| 5 | 110075047 | C | T | SLC25A46 | p.P76L | 0 | 80 | 0.365854 | 41 | WCM365_Z2_1 | Tissue |
| 5 | 172518005 | G | T | CREBRF | p.E275* | 0 | 134 | 0.064516 | 186 | WCM365_Z2_1 | Tissue |
| 5 | 83356276 | G | T | EDIL3 | p.S327* | 0 | 82 | 0.097561 | 82 | WCM365_Z2_1 | Tissue |
| 7 | 100240883 | G | A | ACTL6B | p.R423* | 0 | 73 | 0.214286 | 98 | WCM365_Z2_1 | Tissue |
| 7 | 155755684 | C | A | AC021218.2 | p.T54K | 0 | 122 | 0.113821 | 123 | WCM365_Z2_1 | Tissue |
| 7 | 77975264 | C | A | MAGI2 | p.K400N | 0 | 98 | 0.084112 | 107 | WCM365_Z2_1 | Tissue |
| 8 | 110655004 | C | A | SYBU | p.S61I | 0 | 69 | 0.106557 | 122 | WCM365_Z2_1 | Tissue |
| 8 | 140630515 | G | A | KCNK9 | p.R371W | 0 | 63 | 0.073034 | 178 | WCM365_Z2_1 | Tissue |
| 8 | 52321348 | G | A | PXDNL | p.R946* | 0 | 79 | 0.565217 | 69 | WCM365_Z2_1 | Tissue |
| 9 | 100124651 | A | G | CCDC180 | p.K1255R | 0 | 82 | 0.464286 | 84 | WCM365_Z2_1 | Tissue |
| 9 | 32492500 | T | A | DDX58 | p.K154* | 0 | 49 | 0.696078 | 102 | WCM365_Z2_1 | Tissue |
| X | 106028343 | C | T | RNF128 | p.R261C | 0 | 60 | 0.258065 | 62 | WCM365_Z2_1 | Tissue |
| X | 48211539 | G | T | SSX3 | p.P109Q | 0 | 90 | 0.134185 | 313 | WCM365_Z2_1 | Tissue |
| 12 | 51740409 | T | G | CELA1 | p.Y5S | 0 | 33 | 0.175 | 40 | WCM366 | Plasma |
| 12 | 51740410 | A | G | CELA1 | p.Y5H | 0 | 34 | 0.170732 | 41 | WCM366 | Plasma |
| 15 | 43712787 | T | C | TP53BP1 | p.D1461G | 0 | 33 | 0.121951 | 41 | WCM366 | Plasma |
| 19 | 36297954 | G | A | PRODH2 | p.R299W | 0 | 88 | 0.052632 | 95 | WCM366 | Plasma |
| 8 | 100568874 | G | A | VPS13B | p.A1673T | 0 | 37 | 0.071429 | 42 | WCM366 | Plasma |
| X | 118544323 | C | T | SLC25A43 | p.Q230* | 0 | 38 | 0.071429 | 42 | WCM366 | Plasma |
| 1 | 152059178 | A | G | TCHHL1 | p.V327A | 0 | 94 | 0.391892 | 74 | WCM366_X1_1 | Tissue |
| 11 | 5878161 | T | C | OR52E8 | p.T258A | 0 | 52 | 0.306452 | 62 | WCM366_X1_1 | Tissue |
| 11 | 93431553 | C | G | KIAA1731 | p.Q1159E | 0 | 42 | 0.40678 | 59 | WCM366_X1_1 | Tissue |
| 12 | 122260845 | C | T | SETD1B | p.R1454* | 0 | 145 | 0.463415 | 82 | WCM366_X1_1 | Tissue |
| 14 | 70926292 | G | C | ADAM21 | p.L692F | 0 | 50 | 0.521739 | 46 | WCM366_X1_1 | Tissue |
| 16 | 1961776 | C | T | HS3ST6 | p.A282T | 0 | 163 | 0.373134 | 134 | WCM366_X1_1 | Tissue |
| 16 | 56792499 | C | T | NUP93 | p.R77* | 0 | 84 | 0.361702 | 94 | WCM366_X1_1 | Tissue |
| 20 | 2686344 | C | T | EBF4 | p.R87C | 0 | 76 | 0.083333 | 72 | WCM366_X1_1 | Tissue |
| 21 | 34877955 | G | C | GART | p.L880V | 0 | 80 | 0.481481 | 54 | WCM366_X1_1 | Tissue |
| 4 | 169196534 | C | T | DDX60 | p.D756N | 0 | 85 | 0.247525 | 101 | WCM366_X1_1 | Tissue |
| 5 | 1219159 | G | A | SLC6A19 | p.V439I | 0 | 159 | 0.139344 | 122 | WCM366_X1_1 | Tissue |
| 8 | 113564857 | A | T | CSMD3 | p.W1443R | 0 | 39 | 0.162162 | 37 | WCM366_X1_1 | Tissue |
| 8 | 92982906 | G | T | RUNX1T1 | p.Q507K | 0 | 31 | 0.138889 | 36 | WCM366_X1_1 | Tissue |
| 9 | 27056345 | A | G | IFT74 | p.E504G | 0 | 37 | 0.328125 | 64 | WCM366_X1_1 | Tissue |
| 9 | 35675544 | A | G | CA9 | p.Q138R | 0 | 69 | 0.3 | 70 | WCM366_X1_1 | Tissue |
| 11 | 63258442 | A | G | HRASLS5 | p.L22P | 0 | 37 | 0.052632 | 38 | WCM369 | Plasma |
| 15 | 43712787 | T | C | TP53BP1 | p.D1461G | 0 | 32 | 0.090909 | 44 | WCM369 | Plasma |
| 2 | 85554616 | G | C | TGOLN2 | p.T80S | 0 | 37 | 0.053571 | 56 | WCM369 | Plasma |
| 11 | 57563960 | G | A | CTNND1 | p.R151Q | 0 | 43 | 0.090909 | 33 | WCM369_Z2_1 | Tissue |
| 1 | 209785486 | G | A | CAMK1G | p.C422Y | 0 | 109 | 0.164384 | 73 | WCM369_Z2_1 | Tissue |
| 1 | 244217801 | C | T | ZBTB18 | p.S242L | 0 | 54 | 0.069767 | 43 | WCM369_Z2_1 | Tissue |
| 14 | 20823096 | C | T | PARP2 | p.H311Y | 0 | 247 | 0.079245 | 265 | WCM369_Z2_1 | Tissue |
| 14 | 79746833 | G | C | NRXN3 | p.V67L | 0 | 122 | 0.17 | 100 | WCM369_Z2_1 | Tissue |
| 15 | 42361122 | C | T | PLA2G4D | p.A750T | 0 | 68 | 0.103896 | 77 | WCM369_Z2_1 | Tissue |
| 16 | 78458901 | T | A | WWOX | p.L247* | 0 | 171 | 0.148936 | 141 | WCM369_Z2_1 | Tissue |
| 17 | 37263686 | C | T | PLXDC1 | p.G229S | 0 | 120 | 0.146341 | 123 | WCM369_Z2_1 | Tissue |
| 17 | 8349120 | C | A | NDEL1 | p.Q60K | 0 | 122 | 0.075 | 80 | WCM369_Z2_1 | Tissue |
| 19 | 50434121 | C | T | ATF5 | p.A5V | 0 | 81 | 0.133333 | 90 | WCM369_Z2_1 | Tissue |
| 2 | 153488619 | A | T | FMNL2 | p.N258I | 0 | 145 | 0.126667 | 150 | WCM369_Z2_1 | Tissue |
| 3 | 130159551 | C | A | COL6A5 | p.D2123E | 0 | 39 | 0.166667 | 36 | WCM369_Z2_1 | Tissue |
| 6 | 31596004 | G | A | PRRC2A | p.E585K | 0 | 85 | 0.070796 | 113 | WCM369_Z2_1 | Tissue |
| 7 | 27239668 | C | A | HOXA13 | p.R10L | 0 | 71 | 0.090909 | 66 | WCM369_Z2_1 | Tissue |
| X | 107381043 | G | A | ATG4A | p.C186Y | 0 | 36 | 0.194444 | 36 | WCM369_Z2_1 | Tissue |
| 10 | 124189222 | C | G | PLEKHA1 | p.S328C | 0 | 33 | 0.078125 | 64 | WCM4 | Plasma |
| 10 | 5247768 | G | A | AKR1C4 | p.D140N | 0 | 46 | 0.055556 | 72 | WCM4 | Plasma |
| 10 | 64148158 | A | T | ZNF365 | p.E249D | 0 | 38 | 0.185185 | 54 | WCM4 | Plasma |
| 11 | 22384296 | G | A | SLC17A6 | p.G225R | 0 | 41 | 0.058824 | 85 | WCM4 | Plasma |
| 1 | 155152269 | C | T | TRIM46 | p.R483W | 0 | 39 | 0.175 | 80 | WCM4 | Plasma |
| 1 | 181685206 | G | A | CACNA1E | p.R419Q | 0 | 32 | 0.135135 | 74 | WCM4 | Plasma |
| 14 | 47120402 | C | T | RPL10L | p.E180K | 0 | 41 | 0.117647 | 68 | WCM4 | Plasma |
| 14 | 64606669 | G | T | SYNE2 | p.D4952Y | 0 | 53 | 0.144928 | 69 | WCM4 | Plasma |
| 15 | 43769897 | C | A | TP53BP1 | p.Q278H | 0 | 42 | 0.050633 | 79 | WCM4 | Plasma |
| 16 | 28878668 | T | C | SH2B1 | p.L319P | 0 | 37 | 0.119403 | 67 | WCM4 | Plasma |
| 17 | 10355643 | C | A | MYH4 | p.R1118L | 0 | 34 | 0.204545 | 44 | WCM4 | Plasma |
| 19 | 43411209 | C | G | PSG6 | p.G369R | 0 | 64 | 0.090909 | 88 | WCM4 | Plasma |
| 2 | 187703894 | C | A | ZSWIM2 | p.A96S | 0 | 46 | 0.063291 | 79 | WCM4 | Plasma |
| 2 | 24103566 | T | C | ATAD2B | p.R282G | 0 | 46 | 0.183099 | 71 | WCM4 | Plasma |
| 2 | 27322070 | G | T | KHK | p.V191F | 0 | 36 | 0.068182 | 44 | WCM4 | Plasma |
| 3 | 138256090 | G | A | CEP70 | p.R189C | 0 | 39 | 0.107692 | 65 | WCM4 | Plasma |
| 3 | 29910367 | T | A | RBMS3 | p.L219Q | 0 | 37 | 0.107692 | 65 | WCM4 | Plasma |
| 3 | 42794072 | G | A | CCDC13 | p.R170W | 0 | 34 | 0.1 | 80 | WCM4 | Plasma |
| 7 | 27872537 | C | T | JAZF1 | p.R205H | 0 | 48 | 0.054054 | 74 | WCM4 | Plasma |
| 7 | 87175277 | C | T | ABCB1 | p.V597I | 0 | 33 | 0.112676 | 71 | WCM4 | Plasma |
| 8 | 93029459 | G | C | RUNX1T1 | p.T74R | 0 | 51 | 0.052632 | 76 | WCM4 | Plasma |
| 9 | 21409485 | C | A | IFNA8 | p.L104I | 0 | 37 | 0.068966 | 87 | WCM4 | Plasma |
| 9 | 4697857 | G | A | CDC37L1 | p.R242H | 0 | 62 | 0.123457 | 81 | WCM4 | Plasma |
| 10 | 124189222 | C | G | PLEKHA1 | p.S328C | 0 | 55 | 0.101695 | 59 | WCM4_X1_1 | Tissue |
| 10 | 27048040 | A | C | ABI1 | p.F343L | 0 | 70 | 0.075 | 80 | WCM4_X1_1 | Tissue |
| 10 | 49447674 | G | C | FRMPD2 | p.H254Q | 0 | 213 | 0.088106 | 227 | WCM4_X1_1 | Tissue |
| 10 | 5247768 | G | A | AKR1C4 | p.D140N | 0 | 106 | 0.215054 | 93 | WCM4_X1_1 | Tissue |
| 10 | 64148158 | A | T | ZNF365 | p.E249D | 0 | 124 | 0.229358 | 109 | WCM4_X1_1 | Tissue |
| 10 | 85984369 | A | T | LRIT2 | p.C204* | 0 | 68 | 0.164706 | 85 | WCM4_X1_1 | Tissue |
| 1 | 11097836 | T | C | MASP2 | p.N308D | 0 | 132 | 0.27451 | 102 | WCM4_X1_1 | Tissue |
| 11 | 114441964 | A | C | NXPE4 | p.I444S | 0 | 126 | 0.157143 | 140 | WCM4_X1_1 | Tissue |
| 11 | 120831649 | T | C | GRIK4 | p.Y636H | 0 | 126 | 0.080537 | 149 | WCM4_X1_1 | Tissue |
| 11 | 20077385 | T | A | NAV2 | p.N1594K | 0 | 144 | 0.055556 | 90 | WCM4_X1_1 | Tissue |
| 1 | 150525561 | C | G | ADAMTSL4 | p.P89R | 0 | 36 | 0.130435 | 46 | WCM4_X1_1 | Tissue |
| 1 | 155057737 | A | T | EFNA3 | p.N100I | 0 | 115 | 0.096 | 125 | WCM4_X1_1 | Tissue |
| 1 | 155152269 | C | T | TRIM46 | p.R483W | 0 | 31 | 0.333333 | 39 | WCM4_X1_1 | Tissue |
| 1 | 156756703 | G | A | PRCC | p.E274K | 0 | 56 | 0.131148 | 61 | WCM4_X1_1 | Tissue |
| 1 | 181685206 | G | A | CACNA1E | p.R419Q | 0 | 60 | 0.349398 | 83 | WCM4_X1_1 | Tissue |
| 1 | 20639400 | G | A | VWA5B1 | p.V71M | 0 | 188 | 0.113095 | 168 | WCM4_X1_1 | Tissue |
| 1 | 228556463 | T | A | OBSCN | p.I6603N | 0 | 229 | 0.069204 | 289 | WCM4_X1_1 | Tissue |
| 12 | 53580203 | T | G | ZNF740 | p.C134W | 0 | 121 | 0.096491 | 114 | WCM4_X1_1 | Tissue |
| 13 | 61985648 | C | A | PCDH20 | p.E862* | 0 | 41 | 0.177778 | 45 | WCM4_X1_1 | Tissue |
| 13 | 77730258 | G | A | MYCBP2 | p.P2246S | 0 | 90 | 0.065574 | 61 | WCM4_X1_1 | Tissue |
| 14 | 21862459 | C | T | CHD8 | p.R1859H | 0 | 151 | 0.105263 | 133 | WCM4_X1_1 | Tissue |
| 14 | 47120402 | C | T | RPL10L | p.E180K | 0 | 50 | 0.162791 | 43 | WCM4_X1_1 | Tissue |
| 14 | 77576197 | C | G | KIAA1737 | p.S60C | 0 | 63 | 0.112903 | 62 | WCM4_X1_1 | Tissue |
| 15 | 48757768 | C | G | FBN1 | p.D1647H | 0 | 99 | 0.090909 | 88 | WCM4_X1_1 | Tissue |
| 15 | 83718831 | C | T | BTBD1 | p.D220N | 0 | 40 | 0.088235 | 34 | WCM4_X1_1 | Tissue |
| 15 | 95001413 | T | A | MCTP2 | p.D766E | 0 | 66 | 0.095238 | 42 | WCM4_X1_1 | Tissue |
| 16 | 2026856 | C | A | TBL3 | p.T445N | 0 | 88 | 0.095238 | 105 | WCM4_X1_1 | Tissue |
| 16 | 24831573 | C | T | TNRC6A | p.R1732C | 0 | 68 | 0.078947 | 38 | WCM4_X1_1 | Tissue |
| 16 | 28878668 | T | C | SH2B1 | p.L319P | 0 | 46 | 0.116279 | 43 | WCM4_X1_1 | Tissue |
| 16 | 30079982 | G | A | ALDOA | p.V114I | 0 | 60 | 0.175824 | 91 | WCM4_X1_1 | Tissue |
| 16 | 3108217 | C | A | MMP25 | p.L348M | 0 | 111 | 0.082569 | 109 | WCM4_X1_1 | Tissue |
| 16 | 3406560 | C | A | OR2C1 | p.T207N | 0 | 185 | 0.190184 | 163 | WCM4_X1_1 | Tissue |
| 16 | 55899940 | C | T | CES5A | p.G214S | 0 | 37 | 0.227273 | 44 | WCM4_X1_1 | Tissue |
| 16 | 57758653 | G | A | CCDC135 | p.R555H | 0 | 198 | 0.211765 | 170 | WCM4_X1_1 | Tissue |
| 17 | 36492868 | C | T | GPR179 | p.R407Q | 0 | 50 | 0.192308 | 52 | WCM4_X1_1 | Tissue |
| 17 | 36517603 | G | C | SOCS7 | p.K266N | 0 | 48 | 0.06383 | 47 | WCM4_X1_1 | Tissue |
| 18 | 28711744 | C | A | DSC1 | p.G767V | 0 | 109 | 0.051546 | 97 | WCM4_X1_1 | Tissue |
| 18 | 2891006 | T | G | EMILIN2 | p.L294R | 0 | 77 | 0.0625 | 64 | WCM4_X1_1 | Tissue |
| 1 | 91177999 | G | A | BARHL2 | p.P345L | 0 | 83 | 0.108108 | 74 | WCM4_X1_1 | Tissue |
| 19 | 15083650 | G | A | SLC1A6 | p.R25W | 0 | 60 | 0.2 | 60 | WCM4_X1_1 | Tissue |
| 19 | 15990654 | C | T | CYP4F2 | p.R390Q | 0 | 127 | 0.237624 | 101 | WCM4_X1_1 | Tissue |
| 19 | 39760242 | A | T | IFNL2 | p.T129S | 0 | 194 | 0.235294 | 204 | WCM4_X1_1 | Tissue |
| 19 | 42873052 | A | G | MEGF8 | p.D2180G | 0 | 224 | 0.117647 | 221 | WCM4_X1_1 | Tissue |
| 19 | 43411209 | C | G | PSG6 | p.G369R | 0 | 63 | 0.176471 | 51 | WCM4_X1_1 | Tissue |
| 20 | 60897408 | C | T | LAMA5 | p.C2088Y | 0 | 56 | 0.470588 | 68 | WCM4_X1_1 | Tissue |
| 2 | 179462516 | A | G | TTN | p.V17457A | 0 | 148 | 0.050847 | 118 | WCM4_X1_1 | Tissue |
| 2 | 17955554 | A | C | GEN1 | p.K363T | 0 | 78 | 0.121951 | 41 | WCM4_X1_1 | Tissue |
| 2 | 187703894 | C | A | ZSWIM2 | p.A96S | 0 | 75 | 0.148936 | 47 | WCM4_X1_1 | Tissue |
| 22 | 24509535 | C | T | CABIN1 | p.R1374* | 0 | 108 | 0.230769 | 91 | WCM4_X1_1 | Tissue |
| 2 | 24103566 | T | C | ATAD2B | p.R282G | 0 | 67 | 0.355556 | 45 | WCM4_X1_1 | Tissue |
| 3 | 125855680 | A | T | ALDH1L1 | p.L424H | 0 | 62 | 0.068493 | 73 | WCM4_X1_1 | Tissue |
| 3 | 138256090 | G | A | CEP70 | p.R189C | 0 | 158 | 0.106977 | 215 | WCM4_X1_1 | Tissue |
| 3 | 156867735 | T | C | CCNL1 | p.E304G | 0 | 46 | 0.3 | 60 | WCM4_X1_1 | Tissue |
| 3 | 173997355 | G | A | NLGN1 | p.E522K | 0 | 147 | 0.068783 | 189 | WCM4_X1_1 | Tissue |
| 3 | 183907668 | A | G | ABCF3 | p.N447S | 0 | 127 | 0.364055 | 217 | WCM4_X1_1 | Tissue |
| 3 | 29910367 | T | A | RBMS3 | p.L219Q | 0 | 84 | 0.092105 | 76 | WCM4_X1_1 | Tissue |
| 3 | 49050727 | G | A | WDR6 | p.R587H | 0 | 164 | 0.052632 | 152 | WCM4_X1_1 | Tissue |
| 4 | 17660050 | C | T | FAM184B | p.A654T | 0.00641 | 156 | 0.270833 | 144 | WCM4_X1_1 | Tissue |
| 4 | 46307612 | C | T | GABRA2 | p.G226R | 0 | 85 | 0.206897 | 58 | WCM4_X1_1 | Tissue |
| 4 | 6598902 | C | T | MAN2B2 | p.R374* | 0 | 60 | 0.204545 | 88 | WCM4_X1_1 | Tissue |
| 4 | 7802247 | C | A | AFAP1 | p.E396D | 0 | 33 | 0.078947 | 38 | WCM4_X1_1 | Tissue |
| 6 | 145149949 | A | C | UTRN | p.N3208T | 0 | 62 | 0.136364 | 44 | WCM4_X1_1 | Tissue |
| 7 | 127224623 | T | G | GCC1 | p.E205A | 0 | 222 | 0.075145 | 173 | WCM4_X1_1 | Tissue |
| 7 | 23871852 | A | C | STK31 | p.K976T | 0 | 47 | 0.121212 | 33 | WCM4_X1_1 | Tissue |
| 7 | 87175277 | C | T | ABCB1 | p.V597I | 0 | 99 | 0.202247 | 89 | WCM4_X1_1 | Tissue |
| 7 | 99711692 | G | C | TAF6 | p.I47M | 0 | 131 | 0.149123 | 114 | WCM4_X1_1 | Tissue |
| 8 | 142264769 | C | T | RP11- | p.R16W | 0 | 99 | 0.068627 | 102 | WCM4_X1_1 | Tissue |
| | | | | 10J21.3 | | | | | | | |
| 8 | 2830812 | G | T | CSMD1 | p.P2918H | 0 | 198 | 0.253886 | 193 | WCM4_X1_1 | Tissue |
| 8 | 93029459 | G | C | RUNX1T1 | p.T74R | 0 | 125 | 0.113333 | 150 | WCM4_X1_1 | Tissue |
| 9 | 131899885 | G | T | PPP2R4 | p.E269* | 0 | 52 | 0.082353 | 85 | WCM4_X1_1 | Tissue |
| 9 | 4697857 | G | A | CDC37L1 | p.R242H | 0 | 106 | 0.333333 | 81 | WCM4_X1_1 | Tissue |
| 9 | 97062956 | G | T | ZNF169 | p.R372S | 0 | 81 | 0.217391 | 69 | WCM4_X1_1 | Tissue |
| 9 | 98209630 | C | T | PTCH1 | p.R1303H | 0 | 36 | 0.111111 | 45 | WCM4_X1_1 | Tissue |
| X | 230852 | T | C | GTPBP6 | p.Y12C | 0 | 133 | 0.062937 | 143 | WCM4_X1_1 | Tissue |
| X | 47428973 | G | A | ARAF | p.G446S | 0 | 71 | 0.181818 | 77 | WCM4_X1_1 | Tissue |
| 1 | 46745215 | T | C | LRRC41 | p.M698V | 0 | 39 | 0.130435 | 46 | WCM63 | Plasma |
| 5 | 109961101 | T | G | TMEM232 | p.Y212S | 0 | 45 | 0.131579 | 38 | WCM63 | Plasma |
| 17 | 58740529 | C | A | PPM1D | p.C478* | 0 | 75 | 0.068966 | 87 | WCM63_X1_1 | Tissue |
| 10 | 875667 | T | G | LARP4B | p.K290T | 0 | 91 | 0.458333 | 48 | WCM63_Z1_1 | Tissue |
| 11 | 31812342 | G | A | PAX6 | p.R367W | 0 | 173 | 0.448598 | 107 | WCM63_Z1_1 | Tissue |
| 11 | 36615571 | C | A | RAG2 | p.V50L | 0 | 133 | 0.081081 | 74 | WCM63_Z1_1 | Tissue |
| 1 | 204429736 | A | G | PIK3C2B | p.I455T | 0 | 123 | 0.661417 | 127 | WCM63_Z1_1 | Tissue |
| 12 | 10978862 | G | A | TAS2R10 | p.R3C | 0 | 152 | 0.05988 | 167 | WCM63_Z1_1 | Tissue |
| 12 | 49956852 | C | T | MCRS1 | p.R246H | 0 | 95 | 0.3 | 100 | WCM63_Z1_1 | Tissue |
| 1 | 42915676 | C | A | ZMYND12 | p.Q55H | 0 | 86 | 0.0625 | 48 | WCM63_Z1_1 | Tissue |
| 1 | 46745215 | T | C | LRRC41 | p.M698V | 0 | 56 | 0.418605 | 43 | WCM63_Z1_1 | Tissue |
| 15 | 65115985 | G | T | PIF1 | p.P184T | 0 | 66 | 0.073171 | 41 | WCM63_Z1_1 | Tissue |
| 17 | 4619316 | G | A | ARRB2 | p.V35M | 0 | 318 | 0.096573 | 321 | WCM63_Z1_1 | Tissue |
| 17 | 7733675 | C | T | DNAH2 | p.R3971C | 0 | 40 | 0.475 | 40 | WCM63_Z1_1 | Tissue |
| 17 | 79078454 | A | G | BAIAP2 | p.T403A | 0 | 182 | 0.081897 | 232 | WCM63_Z1_1 | Tissue |
| 19 | 58928537 | G | A | ZNF584 | p.E218K | 0 | 211 | 0.365217 | 230 | WCM63_Z1_1 | Tissue |
| 20 | 25255333 | G | A | PYGB | p.D212N | 0 | 40 | 0.06383 | 47 | WCM63_Z1_1 | Tissue |
| 21 | 43298859 | C | T | PRDM15 | p.V120I | 0 | 79 | 0.61039 | 77 | WCM63_Z1_1 | Tissue |
| 2 | 170428478 | A | T | FASTKD1 | p.I21N | 0 | 71 | 0.416667 | 60 | WCM63_Z1_1 | Tissue |
| 2 | 173955871 | C | A | MLTK | p.Q38K | 0 | 80 | 0.134831 | 89 | WCM63_Z1_1 | Tissue |
| 2 | 200137128 | C | T | SATB2 | p.V670M | 0 | 218 | 0.668085 | 235 | WCM63_Z1_1 | Tissue |
| 3 | 27243026 | C | T | NEK10 | p.E756K | 0 | 206 | 0.257812 | 128 | WCM63_Z1_1 | Tissue |
| 3 | 57327915 | G | A | DNAH12 | p.T3058M | 0 | 84 | 0.246377 | 69 | WCM63_Z1_1 | Tissue |
| 4 | 126370155 | G | C | FAT4 | p.V2662L | 0 | 121 | 0.514286 | 140 | WCM63_Z1_1 | Tissue |
| 4 | 2515449 | G | A | RNF4 | p.C159Y | 0 | 45 | 0.388889 | 36 | WCM63_Z1_1 | Tissue |
| 4 | 4249835 | G | A | TMEM128 | p.P32L | 0 | 56 | 0.55814 | 43 | WCM63_Z1_1 | Tissue |
| 5 | 167887678 | C | G | WWC1 | p.S949R | 0 | 130 | 0.476636 | 107 | WCM63_Z1_1 | Tissue |
| 5 | 180278443 | C | G | ZFP62 | p.E18Q | 0 | 157 | 0.09009 | 111 | WCM63_Z1_1 | Tissue |
| 6 | 160679519 | G | A | SLC22A2 | p.R91C | 0 | 119 | 0.305263 | 95 | WCM63_Z1_1 | Tissue |
| 7 | 147600770 | G | T | CNTNAP2 | p.D738Y | 0 | 47 | 0.285714 | 35 | WCM63_Z1_1 | Tissue |
| 9 | 120053634 | G | A | ASTN2 | p.Q201* | 0 | 80 | 0.323944 | 71 | WCM63_Z1_1 | Tissue |
| X | 114424515 | G | T | RBMXL3 | p.A171S | 0 | 48 | 0.066667 | 45 | WCM63_Z1_1 | Tissue |
| 2 | 148684711 | A | T | ACVR2A | p.L470F | 0 | 49 | 0.076923 | 52 | WCM63_Z19_1 | Tissue |
| 9 | 130224560 | G | T | LRSAM1 | p.V146L | 0 | 61 | 0.071429 | 42 | WCM63_Z19_1 | Tissue |
| 9 | 135139810 | G | A | SETX | p.T2617M | 0 | 44 | 0.085714 | 35 | WCM63_Z19_1 | Tissue |
| 9 | 139743992 | C | A | PHPT1 | p.A37D | 0 | 36 | 0.050847 | 59 | WCM63_Z19_1 | Tissue |
| 10 | 6498648 | G | T | PRKCQ | p.Y545* | 0 | 152 | 0.182692 | 104 | WCM63_Z19_2 | Tissue |
| 10 | 875667 | T | G | LARP4B | p.K290T | 0 | 91 | 0.327586 | 58 | WCM63_Z19_2 | Tissue |
| 11 | 30925144 | C | A | DCDC1 | p.L1465F | 0 | 90 | 0.115942 | 69 | WCM63_Z19_2 | Tissue |
| 11 | 31812342 | G | A | PAX6 | p.R367W | 0 | 173 | 0.318681 | 182 | WCM63_Z19_2 | Tissue |
| 1 | 204429736 | A | G | PIK3C2B | p.I455T | 0 | 123 | 0.466667 | 60 | WCM63_Z19_2 | Tissue |
| 12 | 109505367 | G | C | USP30 | p.D139H | 0 | 228 | 0.285714 | 217 | WCM63_Z19_2 | Tissue |
| 12 | 109505411 | G | C | USP30 | p.Q153H | 0 | 154 | 0.114458 | 166 | WCM63_Z19_2 | Tissue |
| 12 | 47163207 | C | T | SLC38A4 | p.R435H | 0 | 80 | 0.186813 | 91 | WCM63_Z19_2 | Tissue |
| 12 | 49956852 | C | T | MCRS1 | p.R246H | 0 | 95 | 0.171429 | 70 | WCM63_Z19_2 | Tissue |
| 13 | 61986417 | G | T | PCDH20 | p.Y605* | 0 | 87 | 0.083333 | 36 | WCM63_Z19_2 | Tissue |
| 1 | 46745215 | T | C | LRRC41 | p.M698V | 0 | 56 | 0.214286 | 42 | WCM63_Z19_2 | Tissue |
| 17 | 7749246 | G | T | KDM6B | p.G65V | 0 | 77 | 0.075 | 40 | WCM63_Z19_2 | Tissue |
| 19 | 58928537 | G | A | ZNF584 | p.E218K | 0 | 211 | 0.282486 | 177 | WCM63_Z19_2 | Tissue |
| 20 | 7894922 | C | T | HAO1 | p.R145Q | 0 | 276 | 0.307692 | 91 | WCM63_Z19_2 | Tissue |
| 21 | 43298859 | C | T | PRDM15 | p.V120I | 0 | 79 | 0.342105 | 76 | WCM63_Z19_2 | Tissue |
| 2 | 200137128 | C | T | SATB2 | p.V670M | 0 | 218 | 0.545977 | 174 | WCM63_Z19_2 | Tissue |
| 3 | 27243026 | C | T | NEK10 | p.E756K | 0 | 206 | 0.173611 | 144 | WCM63_Z19_2 | Tissue |
| 4 | 126370155 | G | C | FAT4 | p.V2662L | 0 | 121 | 0.454545 | 66 | WCM63_Z19_2 | Tissue |
| 4 | 2515449 | G | A | RNF4 | p.C159Y | 0 | 45 | 0.421875 | 64 | WCM63_Z19_2 | Tissue |
| 4 | 4249835 | G | A | TMEM128 | p.P32L | 0 | 56 | 0.346154 | 52 | WCM63_Z19_2 | Tissue |
| 9 | 120053634 | G | A | ASTN2 | p.Q201* | 0 | 80 | 0.202128 | 94 | WCM63_Z19_2 | Tissue |
| 10 | 90768668 | C | A | FAS | p.C119* | 0 | 48 | 0.06383 | 47 | WCM63_Z2_1 | Tissue |
| 11 | 118375124 | C | A | KMT2A | p.N2836K | 0 | 74 | 0.078431 | 51 | WCM63_Z2_1 | Tissue |
| 1 | 204429736 | A | G | PIK3C2B | p.I455T | 0 | 71 | 0.395604 | 91 | WCM63_Z2_1 | Tissue |
| 1 | 224345357 | G | T | FBXO28 | p.G339V | 0 | 46 | 0.068182 | 44 | WCM63_Z2_1 | Tissue |
| 12 | 49956852 | C | T | MCRS1 | p.R246H | 0 | 139 | 0.476923 | 130 | WCM63_Z2_1 | Tissue |
| 16 | 51171049 | C | A | SALL1 | p.D1317Y | 0 | 248 | 0.206009 | 233 | WCM63_Z2_1 | Tissue |
| 17 | 7224478 | C | T | NEURL4 | p.G1105S | 0 | 171 | 0.065421 | 107 | WCM63_Z2_1 | Tissue |
| 18 | 44682601 | G | T | IER3IP1 | p.P66T | 0 | 32 | 0.081081 | 37 | WCM63_Z2_1 | Tissue |
| 2 | 200137128 | C | T | SATB2 | p.V670M | 0 | 177 | 0.720721 | 111 | WCM63_Z2_1 | Tissue |
| 2 | 98411451 | C | A | TMEM131 | p.A1110S | 0 | 58 | 0.096154 | 52 | WCM63_Z2_1 | Tissue |
| 4 | 126370155 | G | C | FAT4 | p.V2662L | 0 | 167 | 0.408602 | 186 | WCM63_Z2_1 | Tissue |
| 4 | 2515449 | G | A | RNF4 | p.C159Y | 0 | 45 | 0.390244 | 41 | WCM63_Z2_1 | Tissue |
| 5 | 167887678 | C | G | WWC1 | p.S949R | 0 | 101 | 0.397727 | 88 | WCM63_Z2_1 | Tissue |
| 7 | 107572789 | C | T | LAMB1 | p.E1408K | 0 | 142 | 0.081301 | 123 | WCM63_Z2_1 | Tissue |
| 7 | 143884051 | C | T | ARHGEF35 | p.D476N | 0 | 67 | 0.181818 | 77 | WCM63_Z2_1 | Tissue |
| 7 | 99474164 | G | T | OR2AE1 | p.H165N | 0 | 47 | 0.076923 | 52 | WCM63_Z2_1 | Tissue |
| 10 | 59956376 | G | A | IPMK | p.Q238* | 0 | 67 | 0.227273 | 66 | WCM677 | Plasma |
| 11 | 102986019 | G | C | DYNC2H1 | p.A206P | 0 | 84 | 0.233766 | 77 | WCM677 | Plasma |
| 11 | 10781764 | A | G | CTR9 | p.K213E | 0 | 91 | 0.288889 | 90 | WCM677 | Plasma |
| 11 | 7437381 | A | C | SYT9 | p.T385P | 0 | 55 | 0.260274 | 73 | WCM677 | Plasma |
| 1 | 85431277 | C | A | MCOLN2 | p.W64C | 0 | 83 | 0.317073 | 82 | WCM677 | Plasma |
| 19 | 49459028 | G | T | BAX | p.K57N | 0 | 49 | 0.27451 | 51 | WCM677 | Plasma |
| 20 | 48503379 | G | A | SLC9A8 | p.V528M | 0 | 62 | 0.132075 | 53 | WCM677 | Plasma |
| 21 | 39671379 | A | G | KCNJ15 | p.K66E | 0 | 45 | 0.233333 | 60 | WCM677 | Plasma |
| 2 | 179449648 | C | T | TTN | p.A19933T | 0 | 56 | 0.37037 | 54 | WCM677 | Plasma |
| 2 | 20871119 | C | A | GDF7 | p.D429E | 0 | 31 | 0.282051 | 39 | WCM677 | Plasma |
| 2 | 61417443 | C | T | USP34 | p.R3279Q | 0 | 75 | 0.338028 | 71 | WCM677 | Plasma |
| 2 | 8946477 | C | G | KIDINS220 | p.W176S | 0 | 88 | 0.310811 | 74 | WCM677 | Plasma |
| 3 | 52019234 | G | T | ACY1 | p.A36S | 0 | 40 | 0.076923 | 39 | WCM677 | Plasma |
| 5 | 112328389 | C | G | DCP2 | p.Y118* | 0 | 79 | 0.276316 | 76 | WCM677 | Plasma |
| 5 | 121787096 | G | A | SNCAIP | p.E852K | 0 | 59 | 0.277778 | 72 | WCM677 | Plasma |
| 5 | 177019246 | C | T | TMED9 | p.R11W | 0 | 76 | 0.078947 | 38 | WCM677 | Plasma |
| 5 | 179023579 | C | T | RUFY1 | p.R510W | 0 | 36 | 0.085714 | 35 | WCM677 | Plasma |
| 6 | 144171349 | G | C | LTV1 | p.V131L | 0 | 58 | 0.338462 | 65 | WCM677 | Plasma |
| 6 | 47846116 | G | A | PTCHD4 | p.R822C | 0 | 67 | 0.164706 | 85 | WCM677 | Plasma |
| 6 | 88173748 | G | A | C6orf165 | p.R550H | 0 | 71 | 0.142857 | 49 | WCM677 | Plasma |
| 7 | 42006083 | C | T | GLI3 | p.R863H | 0 | 49 | 0.096774 | 31 | WCM677 | Plasma |
| 7 | 42018209 | G | A | GLI3 | p.H546Y | 0 | 41 | 0.145161 | 62 | WCM677 | Plasma |
| 7 | 47384421 | C | T | TNS3 | p.R861H | 0 | 49 | 0.285714 | 42 | WCM677 | Plasma |
| 9 | 135493798 | G | A | DDX31 | p.R687* | 0 | 43 | 0.1875 | 64 | WCM677 | Plasma |
| 9 | 77473606 | G | A | TRPM6 | p.P31L | 0 | 61 | 0.1 | 70 | WCM677 | Plasma |
| X | 32563321 | G | T | DMD | p.P708H | 0 | 38 | 0.708333 | 48 | WCM677 | Plasma |
| 10 | 98307663 | G | T | TM9SF3 | p.A348D | 0 | 43 | 0.06383 | 47 | WCM677_Z12_1 | Tissue |
| 11 | 102986019 | G | C | DYNC2H1 | p.A206P | 0 | 69 | 0.235294 | 68 | WCM677_Z12_1 | Tissue |
| 11 | 10781764 | A | G | CTR9 | p.K213E | 0 | 31 | 0.4 | 40 | WCM677_Z12_1 | Tissue |
| 11 | 33689540 | C | T | KIAA1549L | p.P1797L | 0 | 51 | 0.409091 | 66 | WCM677_Z12_1 | Tissue |
| 11 | 4407050 | G | A | TRIM21 | p.P298L | 0.003759 | 266 | 0.188153 | 287 | WCM677_Z12_1 | Tissue |
| 11 | 7437381 | A | C | SYT9 | p.T385P | 0 | 39 | 0.375 | 56 | WCM677_Z12_1 | Tissue |
| 12 | 120156534 | G | C | CIT | p.R1283G | 0 | 42 | 0.086957 | 69 | WCM677_Z12_1 | Tissue |
| 12 | 12967155 | C | A | DDX47 | p.Q60K | 0 | 74 | 0.076923 | 39 | WCM677_Z12_1 | Tissue |
| 14 | 24731311 | C | T | TGM1 | p.R83Q | 0 | 156 | 0.245902 | 244 | WCM677_Z12_1 | Tissue |
| 17 | 40282409 | C | T | RAB5C | p.V38I | 0 | 284 | 0.219178 | 365 | WCM677_Z12_1 | Tissue |
| 19 | 49459028 | G | T | BAX | p.K57N | 0 | 95 | 0.561151 | 139 | WCM677_Z12_1 | Tissue |
| 20 | 48503379 | G | A | SLC9A8 | p.V528M | 0 | 99 | 0.351145 | 131 | WCM677_Z12_1 | Tissue |
| 21 | 39671379 | A | G | KCNJ15 | p.K66E | 0 | 63 | 0.421053 | 95 | WCM677_Z12_1 | Tissue |
| 2 | 179449648 | C | T | TTN | p.A19933T | 0 | 34 | 0.666667 | 48 | WCM677_Z12_1 | Tissue |
| 2 | 20871119 | C | A | GDF7 | p.D429E | 0 | 140 | 0.556701 | 194 | WCM677_Z12_1 | Tissue |
| 2 | 232100020 | G | A | ARMC9 | p.A236T | 0 | 207 | 0.079646 | 113 | WCM677_Z12_1 | Tissue |
| 2 | 61417443 | C | T | USP34 | p.R3279Q | 0 | 52 | 0.47191 | 89 | WCM677_Z12_1 | Tissue |
| 2 | 8946477 | C | G | KIDINS220 | p.W176S | 0 | 93 | 0.52 | 125 | WCM677_Z12_1 | Tissue |
| 3 | 126736332 | G | A | PLXNA1 | p.R1114H | 0 | 38 | 0.338028 | 71 | WCM677_Z12_1 | Tissue |
| 3 | 168838970 | G | A | MECOM | p.R148W | 0 | 90 | 0.379747 | 79 | WCM677_Z12_1 | Tissue |
| 5 | 112328389 | C | G | DCP2 | p.Y118* | 0 | 39 | 0.40678 | 59 | WCM677_Z12_1 | Tissue |
| 5 | 121787096 | G | A | SNCAIP | p.E852K | 0 | 81 | 0.555556 | 108 | WCM677_Z12_1 | Tissue |
| 5 | 79031102 | A | C | CMYA5 | p.K2172Q | 0 | 31 | 0.69697 | 33 | WCM677_Z12_1 | Tissue |
| 6 | 47846116 | G | A | PTCHD4 | p.R822C | 0 | 136 | 0.280374 | 107 | WCM677_Z12_1 | Tissue |
| 7 | 47384421 | C | T | TNS3 | p.R861H | 0 | 88 | 0.46875 | 128 | WCM677_Z12_1 | Tissue |
| 9 | 135493798 | G | A | DDX31 | p.R687* | 0 | 164 | 0.113537 | 229 | WCM677_Z12_1 | Tissue |
| 9 | 77473606 | G | A | TRPM6 | p.P31L | 0 | 34 | 0.096154 | 52 | WCM677_Z12_1 | Tissue |
| 11 | 102986019 | G | C | DYNC2H1 | p.A206P | 0 | 69 | 0.380952 | 63 | WCM677_Z16_1 | Tissue |
| 11 | 10781764 | A | G | CTR9 | p.K213E | 0 | 31 | 0.6 | 35 | WCM677_Z16_1 | Tissue |
| 11 | 33689540 | C | T | KIAA1549L | p.P1797L | 0 | 51 | 0.491228 | 57 | WCM677_Z16_1 | Tissue |
| 11 | 4407050 | G | A | TRIM21 | p.P298L | 0.003759 | 266 | 0.210345 | 290 | WCM677_Z16_1 | Tissue |
| 11 | 7437381 | A | C | SYT9 | p.T385P | 0 | 39 | 0.349206 | 63 | WCM677_Z16_1 | Tissue |
| 16 | 88951490 | C | G | CBFA2T3 | p.D361H | 0 | 253 | 0.469799 | 149 | WCM677_Z16_1 | Tissue |
| 19 | 49459028 | G | T | BAX | p.K57N | 0 | 95 | 0.385965 | 171 | WCM677_Z16_1 | Tissue |
| 20 | 48503379 | G | A | SLC9A8 | p.V528M | 0 | 99 | 0.377358 | 159 | WCM677_Z16_1 | Tissue |
| 21 | 39671379 | A | G | KCNJ15 | p.K66E | 0 | 63 | 0.537736 | 106 | WCM677_Z16_1 | Tissue |
| 2 | 179449648 | C | T | TTN | p.A19933T | 0 | 34 | 0.631579 | 38 | WCM677_Z16_1 | Tissue |
| 2 | 20871119 | C | A | GDF7 | p.D429E | 0 | 140 | 0.59116 | 181 | WCM677_Z16_1 | Tissue |
| 2 | 61417443 | C | T | USP34 | p.R3279Q | 0 | 52 | 0.471429 | 70 | WCM677_Z16_1 | Tissue |
| 2 | 8946477 | C | G | KIDINS220 | p.W176S | 0 | 93 | 0.475728 | 103 | WCM677_Z16_1 | Tissue |
| 3 | 126736332 | G | A | PLXNA1 | p.R1114H | 0 | 38 | 0.45 | 60 | WCM677_Z16_1 | Tissue |
| 3 | 168838970 | G | A | MECOM | p.R148W | 0 | 90 | 0.325926 | 135 | WCM677_Z16_1 | Tissue |
| 3 | 58552383 | C | T | FAM107A | p.E123K | 0 | 189 | 0.167785 | 149 | WCM677_Z16_1 | Tissue |
| 5 | 112328389 | C | G | DCP2 | p.Y118* | 0 | 39 | 0.2 | 60 | WCM677_Z16_1 | Tissue |
| 5 | 121787096 | G | A | SNCAIP | p.E852K | 0 | 81 | 0.4 | 125 | WCM677_Z16_1 | Tissue |
| 5 | 79031102 | A | C | CMYA5 | p.K2172Q | 0 | 31 | 0.394737 | 38 | WCM677_Z16_1 | Tissue |
| 6 | 47846116 | G | A | PTCHD4 | p.R822C | 0 | 136 | 0.344828 | 116 | WCM677_Z16_1 | Tissue |
| 7 | 47384421 | C | T | TNS3 | p.R861H | 0 | 88 | 0.510204 | 147 | WCM677_Z16_1 | Tissue |
| 9 | 135493798 | G | A | DDX31 | p.R687* | 0 | 164 | 0.251082 | 231 | WCM677_Z16_1 | Tissue |
| 11 | 102986019 | G | C | DYNC2H1 | p.A206P | 0 | 69 | 0.288462 | 52 | WCM677_Z3_1 | Tissue |
| 11 | 10781764 | A | G | CTR9 | p.K213E | 0 | 31 | 0.545455 | 33 | WCM677_Z3_1 | Tissue |
| 11 | 33689540 | C | T | KIAA1549L | p.P1797L | 0 | 51 | 0.35 | 40 | WCM677_Z3_1 | Tissue |
| 11 | 4407050 | G | A | TRIM21 | p.P298L | 0.003759 | 266 | 0.129921 | 254 | WCM677_Z3_1 | Tissue |
| 11 | 5699583 | C | T | TRIM5 | p.E199K | 0 | 68 | 0.131313 | 99 | WCM677_Z3_1 | Tissue |
| 11 | 7437381 | A | C | SYT9 | p.T385P | 0 | 39 | 0.395349 | 43 | WCM677_Z3_1 | Tissue |
| 16 | 47001545 | A | C | DNAJA2 | p.S153A | 0 | 100 | 0.484211 | 95 | WCM677_Z3_1 | Tissue |
| 17 | 32964784 | C | T | TMEM132E | p.R830C | 0 | 53 | 0.081081 | 74 | WCM677_Z3_1 | Tissue |
| 17 | 79867447 | G | T | PCYT2 | p.Q41K | 0 | 36 | 0.075 | 40 | WCM677_Z3_1 | Tissue |
| 19 | 49459028 | G | T | BAX | p.K57N | 0 | 95 | 0.440367 | 109 | WCM677_Z3_1 | Tissue |
| 20 | 472926 | T | C | CSNK2A1 | p.K198R | 0 | 45 | 0.4 | 35 | WCM677_Z3_1 | Tissue |
| 21 | 39671379 | A | G | KCNJ15 | p.K66E | 0 | 63 | 0.382716 | 81 | WCM677_Z3_1 | Tissue |
| 2 | 179449648 | C | T | TTN | p.A19933T | 0 | 34 | 0.789474 | 38 | WCM677_Z3_1 | Tissue |
| 2 | 20871119 | C | A | GDF7 | p.D429E | 0 | 140 | 0.461538 | 169 | WCM677_Z3_1 | Tissue |
| 2 | 54753639 | C | G | SPTBN1 | p.D28E | 0 | 38 | 0.23913 | 46 | WCM677_Z3_1 | Tissue |
| 2 | 61417443 | C | T | USP34 | p.R3279Q | 0 | 52 | 0.397059 | 68 | WCM677_Z3_1 | Tissue |
| 2 | 8946477 | C | G | KIDINS220 | p.W176S | 0 | 93 | 0.4 | 125 | WCM677_Z3_1 | Tissue |
| 3 | 126736332 | G | A | PLXNA1 | p.R1114H | 0 | 38 | 0.254902 | 51 | WCM677_Z3_1 | Tissue |
| 3 | 168838970 | G | A | MECOM | p.R148W | 0 | 90 | 0.329897 | 97 | WCM677_Z3_1 | Tissue |
| 5 | 112328389 | C | G | DCP2 | p.Y118* | 0 | 39 | 0.269231 | 52 | WCM677_Z3_1 | Tissue |
| 5 | 121787096 | G | A | SNCAIP | p.E852K | 0 | 81 | 0.35 | 100 | WCM677_Z3_1 | Tissue |
| 5 | 79031102 | A | C | CMYA5 | p.K2172Q | 0 | 31 | 0.514286 | 35 | WCM677_Z3_1 | Tissue |
| 6 | 47846116 | G | A | PTCHD4 | p.R822C | 0 | 136 | 0.168675 | 83 | WCM677_Z3_1 | Tissue |
| 7 | 47384421 | C | T | TNS3 | p.R861H | 0 | 88 | 0.383721 | 86 | WCM677_Z3_1 | Tissue |
| X | 128710479 | C | A | OCRL | p.R689S | 0 | 47 | 0.054545 | 55 | WCM677_Z3_1 | Tissue |
| X | 21627460 | G | A | CNKSR2 | p.R806Q | 0 | 97 | 0.275862 | 87 | WCM677_Z3_1 | Tissue |
| 11 | 102986019 | G | C | DYNC2H1 | p.A206P | 0 | 69 | 0.155172 | 58 | WCM677_Z6_1 | Tissue |
| 11 | 32956037 | C | A | QSER1 | p.T949K | 0 | 42 | 0.050847 | 59 | WCM677_Z6_1 | Tissue |
| 11 | 33689540 | C | T | KIAA1549L | p.P1797L | 0 | 51 | 0.396226 | 53 | WCM677_Z6_1 | Tissue |
| 11 | 4407050 | G | A | TRIM21 | p.P298L | 0.003759 | 266 | 0.164474 | 304 | WCM677_Z6_1 | Tissue |
| 11 | 5699583 | C | T | TRIM5 | p.E199K | 0 | 68 | 0.257353 | 136 | WCM677_Z6_1 | Tissue |
| 11 | 7437381 | A | C | SYT9 | p.T385P | 0 | 39 | 0.612245 | 49 | WCM677_Z6_1 | Tissue |
| 19 | 12812628 | T | C | TNPO2 | p.K817E | 0 | 285 | 0.164179 | 268 | WCM677_Z6_1 | Tissue |
| 19 | 49459028 | G | T | BAX | p.K57N | 0 | 95 | 0.485075 | 134 | WCM677_Z6_1 | Tissue |
| 20 | 472926 | T | C | CSNK2A1 | p.K198R | 0 | 45 | 0.363636 | 33 | WCM677_Z6_1 | Tissue |
| 21 | 39671379 | A | G | KCNJ15 | p.K66E | 0 | 63 | 0.483871 | 124 | WCM677_Z6_1 | Tissue |
| 2 | 179449648 | C | T | TTN | p.A19933T | 0 | 34 | 0.666667 | 39 | WCM677_Z6_1 | Tissue |
| 2 | 20871119 | C | A | GDF7 | p.D429E | 0 | 140 | 0.503067 | 163 | WCM677_Z6_1 | Tissue |
| 2 | 25799751 | C | T | DTNB | p.G278S | 0 | 65 | 0.24 | 75 | WCM677_Z6_1 | Tissue |
| 2 | 54753639 | C | G | SPTBN1 | p.D28E | 0 | 38 | 0.340909 | 44 | WCM677_Z6_1 | Tissue |
| 2 | 61417443 | C | T | USP34 | p.R3279Q | 0 | 52 | 0.646154 | 65 | WCM677_Z6_1 | Tissue |
| 2 | 8946477 | C | G | KIDINS220 | p.W176S | 0 | 93 | 0.632479 | 117 | WCM677_Z6_1 | Tissue |
| 3 | 126736332 | G | A | PLXNA1 | p.R1114H | 0 | 38 | 0.21519 | 79 | WCM677_Z6_1 | Tissue |
| 3 | 168838970 | G | A | MECOM | p.R148W | 0 | 90 | 0.378205 | 156 | WCM677_Z6_1 | Tissue |
| 4 | 153897264 | C | T | FHDC1 | p.R941C | 0 | 101 | 0.060403 | 149 | WCM677_Z6_1 | Tissue |
| 5 | 112328389 | C | G | DCP2 | p.Y118* | 0 | 39 | 0.129032 | 62 | WCM677_Z6_1 | Tissue |
| 5 | 121787096 | G | A | SNCAIP | p.E852K | 0 | 81 | 0.276119 | 134 | WCM677_Z6_1 | Tissue |
| 5 | 31483728 | C | A | DROSHA | p.L668F | 0 | 36 | 0.145833 | 48 | WCM677_Z6_1 | Tissue |
| 5 | 79031102 | A | C | CMYA5 | p.K2172Q | 0 | 31 | 0.488372 | 43 | WCM677_Z6_1 | Tissue |
| 6 | 47846116 | G | A | PTCHD4 | p.R822C | 0 | 136 | 0.130769 | 130 | WCM677_Z6_1 | Tissue |
| 7 | 47384421 | C | T | TNS3 | p.R861H | 0 | 88 | 0.397163 | 141 | WCM677_Z6_1 | Tissue |
| X | 70836268 | G | A | CXCR3 | p.R352C | 0 | 37 | 0.061224 | 49 | WCM677_Z6_1 | Tissue |
| 11 | 102986019 | G | C | DYNC2H1 | p.A206P | 0 | 69 | 0.255319 | 47 | WCM677_Z7_1 | Tissue |
| 11 | 14540549 | G | T | PSMA1 | p.S14R | 0 | 41 | 0.065217 | 46 | WCM677_Z7_1 | Tissue |
| 11 | 33689540 | C | T | KIAA1549L | p.P1797L | 0 | 51 | 0.487805 | 41 | WCM677_Z7_1 | Tissue |
| 11 | 4407050 | G | A | TRIM21 | p.P298L | 0.003759 | 266 | 0.175 | 240 | WCM677_Z7_1 | Tissue |
| 11 | 5699583 | C | T | TRIM5 | p.E199K | 0 | 68 | 0.145833 | 144 | WCM677_Z7_1 | Tissue |
| 11 | 7437381 | A | C | SYT9 | p.T385P | 0 | 39 | 0.615385 | 52 | WCM677_Z7_1 | Tissue |
| 16 | 47001545 | A | C | DNAJA2 | p.S153A | 0 | 100 | 0.519685 | 127 | WCM677_Z7_1 | Tissue |
| 19 | 49459028 | G | T | BAX | p.K57N | 0 | 95 | 0.435714 | 140 | WCM677_Z7_1 | Tissue |
| 20 | 472926 | T | C | CSNK2A1 | p.K198R | 0 | 45 | 0.382353 | 34 | WCM677_Z7_1 | Tissue |
| 21 | 39671379 | A | G | KCNJ15 | p.K66E | 0 | 63 | 0.486726 | 113 | WCM677_Z7_1 | Tissue |
| 2 | 179449648 | C | T | TTN | p.A19933T | 0 | 34 | 0.756757 | 37 | WCM677_Z7_1 | Tissue |
| 2 | 20871119 | C | A | GDF7 | p.D429E | 0 | 140 | 0.586021 | 186 | WCM677_Z7_1 | Tissue |
| 2 | 54753639 | C | G | SPTBN1 | p.D28E | 0 | 38 | 0.2 | 35 | WCM677_Z7_1 | Tissue |
| 2 | 61417443 | C | T | USP34 | p.R3279Q | 0 | 52 | 0.586207 | 58 | WCM677_Z7_1 | Tissue |
| 2 | 8946477 | C | G | KIDINS220 | p.W176S | 0 | 93 | 0.495146 | 103 | WCM677_Z7_1 | Tissue |
| 3 | 126736332 | G | A | PLXNA1 | p.R1114H | 0 | 38 | 0.351648 | 91 | WCM677_Z7_1 | Tissue |
| 3 | 168838970 | G | A | MECOM | p.R148W | 0 | 90 | 0.317073 | 123 | WCM677_Z7_1 | Tissue |
| 5 | 112328389 | C | G | DCP2 | p.Y118* | 0 | 39 | 0.47619 | 42 | WCM677_Z7_1 | Tissue |
| 5 | 121787096 | G | A | SNCAIP | p.E852K | 0 | 81 | 0.357143 | 126 | WCM677_Z7_1 | Tissue |
| 5 | 79031102 | A | C | CMYA5 | p.K2172Q | 0 | 31 | 0.547619 | 42 | WCM677_Z7_1 | Tissue |
| 6 | 47846116 | G | A | PTCHD4 | p.R822C | 0 | 136 | 0.207792 | 77 | WCM677_Z7_1 | Tissue |
| 7 | 47384421 | C | T | TNS3 | p.R861H | 0 | 88 | 0.558559 | 111 | WCM677_Z7_1 | Tissue |
| 10 | 69948713 | C | T | MYPN | p.R919C | 0 | 96 | 0.074074 | 81 | WCM677_Z9_1 | Tissue |
| 11 | 102986019 | G | C | DYNC2H1 | p.A206P | 0 | 69 | 0.349206 | 63 | WCM677_Z9_1 | Tissue |
| 11 | 10781764 | A | G | CTR9 | p.K213E | 0 | 31 | 0.540541 | 37 | WCM677_Z9_1 | Tissue |
| 11 | 33689540 | C | T | KIAA1549L | p.P1797L | 0 | 51 | 0.390625 | 64 | WCM677_Z9_1 | Tissue |
| 11 | 4407050 | G | A | TRIM21 | p.P298L | 0.003759 | 266 | 0.174377 | 281 | WCM677_Z9_1 | Tissue |
| 11 | 5699583 | C | T | TRIM5 | p.E199K | 0 | 68 | 0.281818 | 110 | WCM677_Z9_1 | Tissue |
| 11 | 7437381 | A | C | SYT9 | p.T385P | 0 | 39 | 0.467742 | 62 | WCM677_Z9_1 | Tissue |
| 16 | 47001545 | A | C | DNAJA2 | p.S153A | 0 | 100 | 0.46281 | 121 | WCM677_Z9_1 | Tissue |
| 17 | 39156020 | C | T | KRTAP3-2 | p.G29E | 0 | 150 | 0.086758 | 219 | WCM677_Z9_1 | Tissue |
| 19 | 49459028 | G | T | BAX | p.K57N | 0 | 95 | 0.454545 | 121 | WCM677_Z9_1 | Tissue |
| 20 | 472926 | T | C | CSNK2A1 | p.K198R | 0 | 45 | 0.378378 | 37 | WCM677_Z9_1 | Tissue |
| 21 | 39671379 | A | G | KCNJ15 | p.K66E | 0 | 63 | 0.631068 | 103 | WCM677_Z9_1 | Tissue |
| 2 | 179449648 | C | T | TTN | p.A19933T | 0 | 34 | 0.621622 | 37 | WCM677_Z9_1 | Tissue |
| 2 | 20871119 | C | A | GDF7 | p.D429E | 0 | 140 | 0.395349 | 172 | WCM677_Z9_1 | Tissue |
| 2 | 54753639 | C | G | SPTBN1 | p.D28E | 0 | 38 | 0.272727 | 44 | WCM677_Z9_1 | Tissue |
| 2 | 61417443 | C | T | USP34 | p.R3279Q | 0 | 52 | 0.410714 | 56 | WCM677_Z9_1 | Tissue |
| 2 | 8946477 | C | G | KIDINS220 | p.W176S | 0 | 93 | 0.557895 | 95 | WCM677_Z9_1 | Tissue |
| 3 | 126736332 | G | A | PLXNA1 | p.R1114H | 0 | 38 | 0.258065 | 62 | WCM677_Z9_1 | Tissue |
| 3 | 168838970 | G | A | MECOM | p.R148W | 0 | 90 | 0.276119 | 134 | WCM677_Z9_1 | Tissue |
| 3 | 38655281 | C | T | SCN5A | p.R219H | 0 | 32 | 0.06 | 50 | WCM677_Z9_1 | Tissue |
| 5 | 112328389 | C | G | DCP2 | p.Y118* | 0 | 39 | 0.375 | 56 | WCM677_Z9_1 | Tissue |
| 5 | 121787096 | G | A | SNCAIP | p.E852K | 0 | 81 | 0.34127 | 126 | WCM677_Z9_1 | Tissue |
| 5 | 79031102 | A | C | CMYA5 | p.K2172Q | 0 | 31 | 0.516129 | 31 | WCM677_Z9_1 | Tissue |
| 6 | 47846116 | G | A | PTCHD4 | p.R822C | 0 | 136 | 0.307018 | 114 | WCM677_Z9_1 | Tissue |
| 7 | 47384421 | C | T | TNS3 | p.R861H | 0 | 88 | 0.436975 | 119 | WCM677_Z9_1 | Tissue |
| 11 | 17548809 | G | A | USH1C | p.L153F | 0 | 41 | 0.608696 | 46 | WCM6_Z5_1 | Tissue |
| 11 | 47364249 | G | C | MYBPC3 | p.R502G | 0 | 81 | 0.181818 | 77 | WCM6_Z5_1 | Tissue |
| 11 | 5172835 | G | T | OR52A1 | p.Y255* | 0 | 100 | 0.346154 | 78 | WCM6_Z5_1 | Tissue |
| 11 | 68153886 | G | A | LRP5 | p.R373Q | 0 | 87 | 0.065789 | 76 | WCM6_Z5_1 | Tissue |
| 1 | 17266398 | G | C | CROCC | p.G540R | 0 | 386 | 0.142857 | 287 | WCM6_Z5_1 | Tissue |
| 1 | 179452274 | C | T | AXDND1 | p.A670V | 0 | 90 | 0.356436 | 101 | WCM6_Z5_1 | Tissue |
| 1 | 179526218 | G | T | NPHS2 | p.H228N | 0 | 483 | 0.38 | 450 | WCM6_Z5_1 | Tissue |
| 1 | 181702853 | G | A | CACNA1E | p.V1077M | 0 | 174 | 0.200893 | 224 | WCM6_Z5_1 | Tissue |
| 11 | 92533273 | G | T | FAT3 | p.R2365I | 0 | 104 | 0.12069 | 58 | WCM6_Z5_1 | Tissue |
| 1 | 215751377 | G | A | KCTD3 | p.R117H | 0 | 32 | 0.394737 | 38 | WCM6_Z5_1 | Tissue |
| 1 | 216246565 | C | A | USH2A | p.V1884F | 0 | 113 | 0.544118 | 136 | WCM6_Z5_1 | Tissue |
| 15 | 54015072 | C | A | WDR72 | p.A63S | 0 | 54 | 0.068182 | 44 | WCM6_Z5_1 | Tissue |
| 16 | 84096991 | A | G | MBTPS1 | p.Y831H | 0 | 229 | 0.842105 | 133 | WCM6_Z5_1 | Tissue |
| 17 | 43003524 | T | C | KIF18B | p.N828S | 0 | 113 | 0.220779 | 154 | WCM6_Z5_1 | Tissue |
| 17 | 7415226 | C | T | POLR2A | p.L1400F | 0 | 102 | 0.315789 | 95 | WCM6_Z5_1 | Tissue |
| 19 | 38857947 | G | A | CATSPERG | p.R922Q | 0 | 50 | 0.536585 | 41 | WCM6_Z5_1 | Tissue |
| 19 | 42754648 | T | G | ERF | p.H31P | 0 | 55 | 0.875 | 32 | WCM6_Z5_1 | Tissue |
| 19 | 50940930 | C | T | MYBPC2 | p.S176L | 0 | 178 | 0.47343 | 207 | WCM6_Z5_1 | Tissue |
| 2 | 217528711 | C | G | IGFBP2 | p.P288A | 0 | 196 | 0.321429 | 168 | WCM6_Z5_1 | Tissue |
| 2 | 236957874 | T | G | AGAP1 | p.V688G | 0 | 96 | 0.371795 | 78 | WCM6_Z5_1 | Tissue |
| 2 | 28772920 | C | T | PLB1 | p.T351I | 0 | 167 | 0.304348 | 115 | WCM6_Z5_1 | Tissue |
| 3 | 197619565 | A | T | IQCG | p.N343K | 0 | 97 | 0.185841 | 113 | WCM6_Z5_1 | Tissue |
| 3 | 48465272 | C | T | PLXNB1 | p.R250Q | 0 | 85 | 0.137615 | 109 | WCM6_Z5_1 | Tissue |
| 3 | 57276166 | T | G | APPL1 | p.S137R | 0 | 65 | 0.588235 | 68 | WCM6_Z5_1 | Tissue |
| 4 | 79207636 | G | A | FRAS1 | p.G493R | 0 | 49 | 0.444444 | 54 | WCM6_Z5_1 | Tissue |
| 5 | 75913928 | G | A | F2RL2 | p.R202W | 0 | 179 | 0.279221 | 154 | WCM6_Z5_1 | Tissue |
| 7 | 139268785 | C | T | HIPK2 | p.V915I | 0 | 120 | 0.649007 | 151 | WCM6_Z5_1 | Tissue |
| 8 | 107719065 | A | G | OXR1 | p.D440G | 0 | 44 | 0.604938 | 81 | WCM6_Z5_1 | Tissue |
| 9 | 101830900 | C | A | COL15A1 | p.H1301N | 0 | 184 | 0.216667 | 240 | WCM6_Z5_1 | Tissue |
| 9 | 136037796 | G | A | GBGT1 | p.H2Y | 0 | 102 | 0.12987 | 154 | WCM6_Z5_1 | Tissue |
| Y | 3447355 | C | A | TGIF2LY | p.Q24K | 0 | 32 | 0.95 | 40 | WCM6_Z5_1 | Tissue |
| 12 | 106853096 | G | T | POLR3B | p.L840F | 0 | 32 | 0.103093 | 97 | WCM7 | Plasma |
| 18 | 48591919 | G | A | SMAD4 | p.R361H | 0 | 37 | 0.07 | 100 | WCM7 | Plasma |
| 3 | 196867113 | C | T | DLG1 | p.S237N | 0 | 41 | 0.074766 | 107 | WCM7 | Plasma |
| 5 | 140475645 | C | A | PCDHB2 | p.T424N | 0 | 44 | 0.145631 | 103 | WCM7 | Plasma |
| 6 | 56880044 | A | T | BEND6 | p.N138Y | 0 | 34 | 0.114583 | 96 | WCM7 | Plasma |
| 9 | 94495617 | G | A | ROR2 | p.R242C | 0 | 56 | 0.099099 | 111 | WCM7 | Plasma |
| 11 | 118220630 | G | T | CD3G | p.M84I | 0 | 34 | 0.083333 | 36 | WCM73 | Plasma |
| 1 | 150680815 | C | T | HORMAD1 | p.R155H | 0 | 56 | 0.078431 | 51 | WCM73 | Plasma |
| 13 | 109318453 | C | T | MYO16 | p.T61M | 0 | 33 | 0.075472 | 53 | WCM73 | Plasma |
| 19 | 38958339 | G | C | RYR1 | p.E1090Q | 0 | 32 | 0.088889 | 45 | WCM73 | Plasma |
| 2 | 158971760 | C | T | UPP2 | p.L110F | 0 | 31 | 0.078947 | 38 | WCM73 | Plasma |
| 2 | 204322299 | T | C | RAPH1 | p.K371R | 0 | 36 | 0.081081 | 37 | WCM73 | Plasma |
| 2 | 220312964 | G | A | SPEG | p.G362R | 0 | 32 | 0.076923 | 39 | WCM73 | Plasma |
| 9 | 15848935 | C | T | CCDC171 | p.T1153M | 0 | 34 | 0.069767 | 43 | WCM73 | Plasma |
| 1 | 1153946 | G | T | SDF4 | p.N268K | 0 | 260 | 0.21875 | 256 | WCM73_X1_1 | Tissue |
| 12 | 56031070 | G | A | OR10P1 | p.R132H | 0 | 594 | 0.153101 | 516 | WCM73_X1_1 | Tissue |
| 12 | 58152463 | G | C | 9-Mar | p.G275A | 0 | 224 | 0.125581 | 215 | WCM73_X1_1 | Tissue |
| 14 | 99924635 | T | C | SETD3 | p.Y219C | 0 | 39 | 0.171429 | 35 | WCM73_X1_1 | Tissue |
| 15 | 74425513 | C | T | ISLR2 | p.R140W | 0 | 68 | 0.210526 | 57 | WCM73_X1_1 | Tissue |
| 19 | 16770221 | T | C | SMIM7 | p.R36G | 0 | 128 | 0.146341 | 123 | WCM73_X1_1 | Tissue |
| 19 | 2403164 | G | T | TMPRSS9 | p.C180F | 0 | 68 | 0.107692 | 65 | WCM73_X1_1 | Tissue |
| 19 | 38958339 | G | C | RYR1 | p.E1090Q | 0 | 112 | 0.151724 | 145 | WCM73_X1_1 | Tissue |
| 19 | 52469245 | G | C | ZNF350 | p.S154C | 0 | 110 | 0.158537 | 82 | WCM73_X1_1 | Tissue |
| 20 | 46301083 | C | T | SULF2 | p.G479S | 0 | 110 | 0.233645 | 107 | WCM73_X1_1 | Tissue |
| 20 | 61513674 | C | T | DIDO1 | p.D1212N | 0 | 114 | 0.191011 | 89 | WCM73_X1_1 | Tissue |
| 2 | 207619823 | G | A | MDH1B | p.R274C | 0 | 77 | 0.142857 | 84 | WCM73_X1_1 | Tissue |
| 2 | 218675010 | G | T | TNS1 | p.P1585T | 0 | 143 | 0.198198 | 111 | WCM73_X1_1 | Tissue |
| 2 | 70408858 | C | T | C2orf42 | p.R87Q | 0 | 119 | 0.204545 | 132 | WCM73_X1_1 | Tissue |
| 3 | 47470079 | C | A | SCAP | p.V112L | 0 | 41 | 0.103448 | 58 | WCM73_X1_1 | Tissue |
| 5 | 140481852 | C | A | PCDHB3 | p.A540D | 0 | 32 | 0.090909 | 33 | WCM73_X1_1 | Tissue |
| 7 | 98555641 | C | T | TRRAP | p.S2083F | 0 | 84 | 0.180851 | 94 | WCM73_X1_1 | Tissue |
| 9 | 131115331 | C | T | SLC27A4 | p.R339C | 0 | 59 | 0.06383 | 47 | WCM73_X1_1 | Tissue |
| X | 102931244 | T | C | MORF4L2 | p.M238V | 0 | 98 | 0.064815 | 108 | WCM73_X1_1 | Tissue |
| X | 125298904 | C | T | DCAP12L2 | p.R335H | 0 | 110 | 0.091954 | 87 | WCM73_X1_1 | Tissue |
| 10 | 48382209 | G | A | RBP3 | p.T1147M | 0 | 38 | 0.109589 | 73 | WCM78 | Plasma |
| 1 | 102271668 | C | T | OLFM3 | p.W241* | 0 | 65 | 0.145455 | 110 | WCM78 | Plasma |
| 12 | 78520960 | A | G | NAV3 | p.R1418G | 0 | 58 | 0.090909 | 99 | WCM78 | Plasma |
| 12 | 95461124 | C | T | NR2C1 | p.E13K | 0 | 53 | 0.093458 | 107 | WCM78 | Plasma |
| 12 | 98993884 | A | C | SLC25A3 | p.T266P | 0 | 35 | 0.096774 | 62 | WCM78 | Plasma |
| 14 | 23900801 | G | A | MYH7 | p.S242F | 0 | 51 | 0.095652 | 115 | WCM78 | Plasma |
| 14 | 79181393 | G | T | NRXN3 | p.C279F | 0 | 41 | 0.051948 | 77 | WCM78 | Plasma |
| 15 | 50535413 | G | T | HDC | p.S390Y | 0 | 33 | 0.129412 | 85 | WCM78 | Plasma |
| 17 | 37680959 | G | A | CDK12 | p.W1043* | 0 | 59 | 0.206186 | 97 | WCM78 | Plasma |
| 17 | 38027738 | C | T | ZPBP2 | p.T89I | 0 | 54 | 0.158879 | 107 | WCM78 | Plasma |
| 17 | 38711861 | C | G | CCR7 | p.K90N | 0 | 49 | 0.159574 | 94 | WCM78 | Plasma |
| 17 | 61628127 | A | G | DCAF7 | p.K30R | 0 | 44 | 0.155844 | 77 | WCM78 | Plasma |
| 17 | 8170770 | G | C | PFAS | p.A1086P | 0 | 47 | 0.070423 | 71 | WCM78 | Plasma |
| 18 | 28720116 | T | A | DSC1 | p.E470V | 0 | 48 | 0.084211 | 95 | WCM78 | Plasma |
| 18 | 346996 | A | C | COLEC12 | p.L209R | 0 | 43 | 0.084337 | 83 | WCM78 | Plasma |
| 19 | 9065391 | A | T | MUC16 | p.V7352E | 0 | 48 | 0.125 | 88 | WCM78 | Plasma |
| 20 | 35563511 | G | A | SAMHD1 | p.L144F | 0 | 49 | 0.087912 | 91 | WCM78 | Plasma |
| 2 | 141108374 | C | A | LRP1B | p.G3962C | 0 | 58 | 0.142857 | 91 | WCM78 | Plasma |
| 2 | 202257645 | C | A | TRAK2 | p.G367V | 0 | 54 | 0.086957 | 69 | WCM78 | Plasma |
| 2 | 210761127 | C | T | UNC80 | p.S1458L | 0 | 46 | 0.054348 | 92 | WCM78 | Plasma |
| 22 | 26830423 | C | T | ASPHD2 | p.T281M | 0 | 56 | 0.15 | 80 | WCM78 | Plasma |
| 2 | 241465794 | G | A | ANKMY1 | p.P252L | 0 | 48 | 0.08642 | 81 | WCM78 | Plasma |
| 22 | 42003846 | G | C | DESI1 | p.I36M | 0 | 43 | 0.053191 | 94 | WCM78 | Plasma |
| 5 | 112884245 | C | G | YTHDC2 | p.Y501* | 0 | 50 | 0.128205 | 78 | WCM78 | Plasma |
| 5 | 89695331 | C | A | CETN3 | p.E102* | 0 | 62 | 0.082474 | 97 | WCM78 | Plasma |
| 6 | 132939067 | C | T | TAAR2 | p.G93E | 0 | 51 | 0.1875 | 80 | WCM78 | Plasma |
| 7 | 100633889 | C | A | MUC12 | p.F158L | 0 | 34 | 0.148649 | 74 | WCM78 | Plasma |
| 8 | 133898711 | C | T | TG | p.A365V | 0 | 37 | 0.055556 | 72 | WCM78 | Plasma |
| 8 | 43024371 | C | T | HGSNAT | p.R235C | 0 | 60 | 0.107843 | 102 | WCM78 | Plasma |
| 10 | 24922371 | C | A | ARHGAP21 | p.G134C | 0 | 48 | 0.082192 | 73 | WCM78_Z1_1 | Tissue |
| 10 | 24922403 | C | A | ARHGAP21 | p.R123L | 0 | 48 | 0.082192 | 73 | WCM78_Z1_1 | Tissue |
| 10 | 48382209 | G | A | RBP3 | p.T1147M | 0 | 65 | 0.283333 | 60 | WCM78_Z1_1 | Tissue |
| 1 | 102271668 | C | T | OLFM3 | p.W241* | 0 | 92 | 0.324324 | 74 | WCM78_Z1_1 | Tissue |
| 11 | 119025562 | G | A | ABCG4 | p.R208H | 0 | 128 | 0.110497 | 181 | WCM78_Z1_1 | Tissue |
| 11 | 130275813 | G | C | ADAMTS8 | p.I770M | 0 | 102 | 0.093023 | 86 | WCM78_Z1_1 | Tissue |
| 12 | 6697048 | G | A | CHD4 | p.A1178V | 0 | 157 | 0.503731 | 268 | WCM78_Z1_1 | Tissue |
| 12 | 78520960 | A | G | NAV3 | p.R1418G | 0 | 140 | 0.418079 | 177 | WCM78_Z1_1 | Tissue |
| 12 | 98993884 | A | C | SLC25A3 | p.T266P | 0 | 87 | 0.306931 | 101 | WCM78_Z1_1 | Tissue |
| 13 | 113496617 | C | T | ATP11A | p.A578V | 0 | 65 | 0.073171 | 82 | WCM78_Z1_1 | Tissue |
| 14 | 23900801 | G | A | MYH7 | p.S242F | 0 | 195 | 0.12619 | 420 | WCM78_Z1_1 | Tissue |
| 15 | 33916174 | C | A | RYR3 | p.Q842K | 0 | 41 | 0.282609 | 46 | WCM78_Z1_1 | Tissue |
| 15 | 50535413 | G | T | HDC | p.S390Y | 0 | 171 | 0.344595 | 148 | WCM78_Z1_1 | Tissue |
| 16 | 16138529 | G | C | ABCC1 | p.Q344H | 0 | 86 | 0.307692 | 156 | WCM78_Z1_1 | Tissue |
| 16 | 28502861 | G | C | CLN3 | p.P23A | 0 | 67 | 0.226415 | 53 | WCM78_Z1_1 | Tissue |
| 16 | 840208 | G | A | CHTF18 | p.G213D | 0 | 36 | 0.172414 | 58 | WCM78_Z1_1 | Tissue |
| 17 | 37680959 | G | A | CDK12 | p.W1043* | 0 | 41 | 0.442623 | 61 | WCM78_Z1_1 | Tissue |
| 17 | 38643491 | G | C | TNS4 | p.P362R | 0 | 206 | 0.050167 | 299 | WCM78_Z1_1 | Tissue |
| 17 | 38711861 | C | G | CCR7 | p.K90N | 0 | 74 | 0.522388 | 134 | WCM78_Z1_1 | Tissue |
| 17 | 59668349 | C | A | NACA2 | p.G65C | 0 | 37 | 0.061224 | 49 | WCM78_Z1_1 | Tissue |
| 18 | 28720116 | T | A | DSC1 | p.E470V | 0 | 423 | 0.224138 | 464 | WCM78_Z1_1 | Tissue |
| 18 | 346996 | A | C | COLEC12 | p.L209R | 0 | 64 | 0.236842 | 76 | WCM78_Z1_1 | Tissue |
| 18 | 70461472 | C | A | NETO1 | p.E173D | 0 | 106 | 0.069444 | 72 | WCM78_Z1_1 | Tissue |
| 19 | 59028764 | C | A | ZBTB45 | p.V93F | 0 | 116 | 0.067227 | 119 | WCM78_Z1_1 | Tissue |
| 20 | 35563511 | G | A | SAMHD1 | p.L144F | 0 | 228 | 0.301435 | 209 | WCM78_Z1_1 | Tissue |
| 20 | 57768580 | C | T | ZNF831 | p.P836S | 0 | 268 | 0.198157 | 217 | WCM78_Z1_1 | Tissue |
| 2 | 141108374 | C | A | LRP1B | p.G3962C | 0 | 75 | 0.149254 | 67 | WCM78_Z1_1 | Tissue |
| 2 | 152382475 | A | T | NEB | p.S5685R | 0 | 108 | 0.095238 | 63 | WCM78_Z1_1 | Tissue |
| 2 | 202257645 | C | A | TRAK2 | p.G367V | 0 | 45 | 0.329114 | 79 | WCM78_Z1_1 | Tissue |
| 2 | 210761127 | C | T | UNC80 | p.S1458L | 0 | 98 | 0.245902 | 122 | WCM78_Z1_1 | Tissue |
| 22 | 26830423 | C | T | ASPHD2 | p.T281M | 0 | 155 | 0.280488 | 164 | WCM78_Z1_1 | Tissue |
| 22 | 31688276 | C | A | PIK3IP1 | p.A19S | 0 | 56 | 0.083333 | 36 | WCM78_Z1_1 | Tissue |
| 2 | 241465794 | G | A | ANKMY1 | p.P252L | 0 | 139 | 0.53211 | 109 | WCM78_Z1_1 | Tissue |
| 22 | 42003846 | G | C | DESI1 | p.I36M | 0 | 52 | 0.190476 | 63 | WCM78_Z1_1 | Tissue |
| 2 | 97403735 | C | T | LMAN2L | p.R86H | 0 | 73 | 0.1 | 60 | WCM78_Z1_1 | Tissue |
| 4 | 187455223 | G | A | MTNR1A | p.R225C | 0 | 93 | 0.211009 | 109 | WCM78_Z1_1 | Tissue |
| 4 | 48492375 | C | T | ZAR1 | p.R23W | 0 | 187 | 0.51751 | 257 | WCM78_Z1_1 | Tissue |
| 5 | 112884245 | C | G | YTHDC2 | p.Y501* | 0 | 56 | 0.367816 | 87 | WCM78_Z1_1 | Tissue |
| 5 | 176004674 | G | A | CDHR2 | p.V463I | 0 | 231 | 0.076233 | 223 | WCM78_Z1_1 | Tissue |
| 6 | 136904799 | T | A | MAP3K5 | p.D1102V | 0 | 194 | 0.105882 | 170 | WCM78_Z1_1 | Tissue |
| 7 | 100633889 | C | A | MUC12 | p.F158L | 0 | 173 | 0.35545 | 211 | WCM78_Z1_1 | Tissue |
| 8 | 110587233 | C | T | SYBU | p.G632R | 0 | 42 | 0.27027 | 37 | WCM78_Z1_1 | Tissue |
| 8 | 113304826 | G | C | CSMD3 | p.P2910A | 0 | 66 | 0.263158 | 95 | WCM78_Z1_1 | Tissue |
| 9 | 140076213 | C | A | ANAPC2 | p.S463I | 0 | 129 | 0.18254 | 126 | WCM78_Z1_1 | Tissue |
| 11 | 17794107 | C | T | KCNC1 | p.P489L | 0 | 74 | 0.264151 | 53 | WCM7_Tissue | Tissue |
| 12 | 106853096 | G | T | POLR3B | p.L840F | 0 | 58 | 0.340426 | 47 | WCM7_Tissue | Tissue |
| 17 | 20156848 | G | A | SPECC1 | p.A877T | 0 | 63 | 0.275 | 40 | WCM7_Tissue | Tissue |
| 17 | 27613283 | C | G | NUFIP2 | p.G577R | 0 | 154 | 0.337662 | 77 | WCM7_Tissue | Tissue |
| 2 | 107049447 | C | T | RGPD3 | p.A805T | 0 | 31 | 0.058824 | 85 | WCM7_Tissue | Tissue |
| 22 | 25007172 | G | A | GGT1 | p.A42T | 0 | 167 | 0.083333 | 36 | WCM7_Tissue | Tissue |
| 3 | 196867113 | C | T | DLG1 | p.S237N | 0 | 47 | 0.2 | 55 | WCM7_Tissue | Tissue |
| 3 | 41266124 | A | G | CTNNB1 | p.T41A | 0 | 59 | 0.392857 | 56 | WCM7_Tissue | Tissue |
| 3 | 56627669 | G | T | CCDC66 | p.V407F | 0 | 37 | 0.083333 | 36 | WCM7_Tissue | Tissue |
| 4 | 39435929 | C | T | KLB | p.R309W | 0 | 100 | 0.685714 | 35 | WCM7_Tissue | Tissue |
| 5 | 140475645 | C | A | PCDHB2 | p.T424N | 0 | 191 | 0.428571 | 98 | WCM7_Tissue | Tissue |
| 5 | 14336754 | G | A | TRIO | p.R655Q | 0 | 94 | 0.319444 | 72 | WCM7_Tissue | Tissue |
| 6 | 56880044 | A | T | BEND6 | p.N138Y | 0 | 80 | 0.290909 | 55 | WCM7_Tissue | Tissue |
| 9 | 94495617 | G | A | ROR2 | p.R242C | 0 | 105 | 0.4 | 35 | WCM7_Tissue | Tissue |
| X | 99662212 | C | T | PCDH19 | p.V462M | 0 | 92 | 0.083333 | 36 | WCM7_Tissue | Tissue |
| 10 | 133948606 | C | T | JAKMIP3 | p.A304V | 0 | 52 | 0.064516 | 62 | WCM80 | Plasma |
| 1 | 151630836 | G | C | SNX27 | p.W223C | 0 | 48 | 0.233333 | 60 | WCM80 | Plasma |
| 12 | 40852541 | C | A | MUC19 | p.L1106I | 0 | 38 | 0.230769 | 39 | WCM80 | Plasma |
| 14 | 102606487 | A | G | WDR20 | p.Y76C | 0 | 53 | 0.088235 | 34 | WCM80 | Plasma |
| 15 | 92459662 | G | A | SLCO3A1 | p.R207Q | 0 | 35 | 0.318182 | 66 | WCM80 | Plasma |
| 17 | 11622752 | G | A | DNAH9 | p.R1885H | 0 | 36 | 0.340909 | 44 | WCM80 | Plasma |
| 17 | 7808965 | C | T | CHD3 | p.R1389W | 0 | 35 | 0.081081 | 37 | WCM80 | Plasma |
| 19 | 14866513 | C | T | EMR2 | p.D457N | 0 | 46 | 0.285714 | 63 | WCM80 | Plasma |
| 19 | 17313977 | G | A | MYO9B | p.V1634M | 0 | 40 | 0.069767 | 43 | WCM80 | Plasma |
| 19 | 18311231 | G | A | RAB3A | p.R85W | 0 | 66 | 0.223684 | 76 | WCM80 | Plasma |
| 19 | 52619693 | G | C | ZNF616 | p.Q242E | 0 | 36 | 0.319149 | 47 | WCM80 | Plasma |
| 2 | 186678471 | A | G | FSIP2 | p.K6676R | 0 | 48 | 0.137255 | 51 | WCM80 | Plasma |
| 2 | 229890686 | G | A | PID1 | p.R139W | 0 | 45 | 0.47619 | 63 | WCM80 | Plasma |
| 22 | 31953032 | C | T | SFI1 | p.R217W | 0 | 50 | 0.066667 | 60 | WCM80 | Plasma |
| 22 | 38824050 | C | T | KCNJ4 | p.V30M | 0 | 41 | 0.088889 | 45 | WCM80 | Plasma |
| 3 | 66396808 | G | C | SLC25A26 | p.Q70H | 0 | 54 | 0.333333 | 63 | WCM80 | Plasma |
| 7 | 150500730 | C | T | TMEM176A | p.T122M | 0 | 50 | 0.369565 | 46 | WCM80 | Plasma |
| 8 | 103663827 | G | A | KLF10 | p.P245S | 0 | 32 | 0.09375 | 32 | WCM80 | Plasma |
| 8 | 113267570 | A | T | CSMD3 | p.C3317S | 0 | 36 | 0.534483 | 58 | WCM80 | Plasma |
| 8 | 77768306 | T | C | ZFHX4 | p.L3050S | 0 | 40 | 0.188679 | 53 | WCM80 | Plasma |
| 9 | 101068579 | C | T | GABBR2 | p.A685T | 0 | 41 | 0.233333 | 60 | WCM80 | Plasma |
| 9 | 1053761 | C | T | DMRT2 | p.R189C | 0 | 50 | 0.262295 | 61 | WCM80 | Plasma |
| 1 | 168282103 | G | T | TBX19 | p.G404C | 0 | 104 | 0.255814 | 86 | WCM80_Z1_1 | Tissue |
| 12 | 40852541 | C | A | MUC19 | p.L1106I | 0 | 177 | 0.320442 | 181 | WCM80_Z1_1 | Tissue |
| 14 | 102606487 | A | G | WDR20 | p.Y76C | 0 | 86 | 0.22619 | 84 | WCM80_Z1_1 | Tissue |
| 14 | 105353809 | C | A | CEP170B | p.S1078Y | 0 | 68 | 0.083333 | 72 | WCM80_Z1_1 | Tissue |
| 15 | 92459662 | G | A | SLCO3A1 | p.R207Q | 0 | 103 | 0.22 | 100 | WCM80_Z1_1 | Tissue |
| 17 | 11622752 | G | A | DNAH9 | p.R1885H | 0 | 206 | 0.242775 | 173 | WCM80_Z1_1 | Tissue |
| 18 | 44036562 | G | A | RNF165 | p.R335Q | 0 | 223 | 0.053892 | 167 | WCM80_Z1_1 | Tissue |
| 19 | 14866513 | C | T | EMR2 | p.D457N | 0 | 71 | 0.393258 | 89 | WCM80_Z1_1 | Tissue |
| 19 | 52619693 | G | C | ZNF616 | p.Q242E | 0 | 62 | 0.402985 | 67 | WCM80_Z1_1 | Tissue |
| 2 | 186678471 | A | G | FSIP2 | p.K6676R | 0 | 70 | 0.359375 | 64 | WCM80_Z1_1 | Tissue |
| 22 | 23230428 | C | G | IGLL5 | p.S65R | 0 | 151 | 0.089888 | 178 | WCM80_Z1_1 | Tissue |
| 2 | 229890686 | G | A | PID1 | p.R139W | 0 | 152 | 0.29771 | 131 | WCM80_Z1_1 | Tissue |
| 3 | 13679261 | C | T | FBLN2 | p.R1133C | 0 | 155 | 0.463087 | 149 | WCM80_Z1_1 | Tissue |
| 3 | 88188671 | G | T | ZNF654 | p.E71* | 0 | 62 | 0.297619 | 84 | WCM80_Z1_1 | Tissue |
| 7 | 150500730 | C | T | TMEM176A | p.T122M | 0 | 142 | 0.363636 | 143 | WCM80_Z1_1 | Tissue |
| 8 | 59059408 | A | G | FAM110B | p.T207A | 0 | 182 | 0.423529 | 170 | WCM80_Z1_1 | Tissue |
| 5 | 109103259 | G | T | MAN2A1 | p.A287S | 0 | 59 | 0.054054 | 74 | WCM89 | Plasma |
| 7 | 148515125 | G | A | EZH2 | p.R357W | 0 | 39 | 0.066667 | 60 | WCM89 | Plasma |
| 11 | 1099262 | A | G | MUC2 | p.E2414G | 0 | 78 | 0.361702 | 94 | WCM89_X1_1 | Tissue |
| 1 | 204226501 | G | A | PLEKHA6 | p.R502* | 0 | 65 | 0.173913 | 92 | WCM89_X1_1 | Tissue |
| 16 | 81397425 | G | T | GAN | p.E371D | 0 | 55 | 0.446429 | 56 | WCM89_X1_1 | Tissue |
| 17 | 40485737 | G | A | STAT3 | p.R335W | 0 | 52 | 0.380952 | 63 | WCM89_X1_1 | Tissue |
| 2 | 179588159 | C | T | TTN | p.R6906H | 0 | 196 | 0.304659 | 279 | WCM89_X1_1 | Tissue |
| 3 | 129297265 | G | T | PLXND1 | p.D751E | 0 | 57 | 0.066667 | 45 | WCM89_X1_1 | Tissue |
| 3 | 188242478 | A | C | LPP | p.E111A | 0 | 79 | 0.234043 | 94 | WCM89_X1_1 | Tissue |
| 3 | 77623761 | C | T | ROBO2 | p.R695* | 0 | 102 | 0.533333 | 120 | WCM89_X1_1 | Tissue |
| 4 | 39343998 | G | A | RFC1 | p.R100W | 0 | 74 | 0.117117 | 111 | WCM89_X1_1 | Tissue |
| 5 | 114961344 | A | C | TMED7 | p.F49C | 0 | 113 | 0.235772 | 123 | WCM89_X1_1 | Tissue |
| 5 | 98234026 | A | C | CHD1 | p.F433L | 0 | 77 | 0.38125 | 160 | WCM89_X1_1 | Tissue |
| 8 | 10465652 | T | C | RP1L1 | p.T1986A | 0 | 43 | 0.116279 | 43 | WCM89_X1_1 | Tissue |
| 11 | 1099262 | A | G | MUC2 | p.E2414G | 0 | 78 | 0.1875 | 64 | WCM89_Z1_1 | Tissue |
| 11 | 3239964 | G | A | MRGPRG | p.P27L | 0 | 94 | 0.327103 | 107 | WCM89_Z1_1 | Tissue |
| 1 | 204226501 | G | A | PLEKHA6 | p.R502* | 0 | 65 | 0.140845 | 71 | WCM89_Z1_1 | Tissue |
| 15 | 40247912 | G | T | EIF2AK4 | p.G229V | 0 | 54 | 0.073529 | 68 | WCM89_Z1_1 | Tissue |
| 15 | 93485061 | A | T | CHD2 | p.E234D | 0 | 52 | 0.171053 | 76 | WCM89_Z1_1 | Tissue |
| 17 | 40485737 | G | A | STAT3 | p.R335W | 0 | 52 | 0.259259 | 54 | WCM89_Z1_1 | Tissue |
| 18 | 77679334 | A | C | PQLC1 | p.F153C | 0 | 60 | 0.076923 | 65 | WCM89_Z1_1 | Tissue |
| 2 | 179588159 | C | T | TTN | p.R6906H | 0 | 196 | 0.210526 | 190 | WCM89_Z1_1 | Tissue |
| 3 | 188242478 | A | C | LPP | p.E111A | 0 | 79 | 0.091954 | 87 | WCM89_Z1_1 | Tissue |
| 3 | 77623761 | C | T | ROBO2 | p.R695* | 0 | 102 | 0.161905 | 105 | WCM89_Z1_1 | Tissue |
| 5 | 114961344 | A | C | TMED7 | p.F49C | 0 | 113 | 0.229008 | 131 | WCM89_Z1_1 | Tissue |
| 5 | 98234026 | A | C | CHD1 | p.F433L | 0 | 77 | 0.23 | 100 | WCM89_Z1_1 | Tissue |
| 6 | 29408478 | C | A | OR10C1 | p.S229Y | 0 | 199 | 0.118812 | 202 | WCM89_Z1_1 | Tissue |
| 8 | 17132424 | C | G | VPS37A | p.S200* | 0 | 104 | 0.265306 | 98 | WCM89_Z1_1 | Tissue |
| 10 | 120928786 | C | T | PRDX3 | p.R207Q | 0 | 64 | 0.092308 | 65 | WCM89_Z12_1 | Tissue |
| 10 | 73475820 | C | T | C10orf105 | p.R92H | 0 | 56 | 0.053333 | 75 | WCM89_Z12_1 | Tissue |
| 11 | 117063322 | C | G | SIDT2 | p.L672V | 0 | 54 | 0.114754 | 61 | WCM89_Z12_1 | Tissue |
| 11 | 20417415 | C | A | PRMT3 | p.T156K | 0 | 46 | 0.078947 | 38 | WCM89_Z12_1 | Tissue |
| 11 | 61053894 | C | T | VWCE | p.E145K | 0 | 75 | 0.0625 | 80 | WCM89_Z12_1 | Tissue |
| 1 | 20417103 | G | A | PLA2G5 | p.R112Q | 0 | 188 | 0.055172 | 145 | WCM89_Z12_1 | Tissue |
| 12 | 105464484 | C | T | ALDH1L2 | p.V98M | 0 | 257 | 0.149466 | 281 | WCM89_Z12_1 | Tissue |
| 15 | 81592812 | C | A | IL16 | p.L1049I | 0 | 48 | 0.071429 | 42 | WCM89_Z12_1 | Tissue |
| 16 | 19083386 | G | T | COQ7 | p.Q70H | 0 | 234 | 0.052632 | 190 | WCM89_Z12_1 | Tissue |
| 2 | 155099364 | G | T | GALNT13 | p.C211F | 0 | 130 | 0.258065 | 124 | WCM89_Z12_1 | Tissue |
| 2 | 204036714 | C | T | NBEAL1 | p.R2027* | 0 | 56 | 0.085106 | 47 | WCM89_Z12_1 | Tissue |
| 22 | 39631807 | G | A | PDGFB | p.R46C | 0 | 142 | 0.074766 | 107 | WCM89_Z12_1 | Tissue |
| 3 | 179525570 | C | T | PEX5L | p.R523H | 0 | 107 | 0.397059 | 68 | WCM89_Z12_1 | Tissue |
| 3 | 49163577 | G | A | LAMB2 | p.R723C | 0 | 46 | 0.096774 | 31 | WCM89_Z12_1 | Tissue |
| 3 | 52876927 | G | A | TMEM110 | p.T223M | 0 | 85 | 0.171875 | 64 | WCM89_Z12_1 | Tissue |
| 5 | 77511916 | C | T | AP3B1 | p.R250Q | 0 | 68 | 0.06 | 50 | WCM89_Z12_1 | Tissue |
| 6 | 35762930 | T | C | CLPS | p.K111R | 0 | 85 | 0.135417 | 96 | WCM89_Z12_1 | Tissue |
| 8 | 143546070 | G | A | BAI1 | p.V171M | 0 | 35 | 0.12 | 50 | WCM89_Z12_1 | Tissue |
| 9 | 116858674 | C | T | KIF12 | p.R106H | 0 | 59 | 0.090909 | 33 | WCM89_Z12_1 | Tissue |
| 9 | 134323084 | G | A | PRRC2B | p.R338H | 0 | 36 | 0.28125 | 32 | WCM89_Z12_1 | Tissue |
| 10 | 124596398 | G | A | CUZD1 | p.R256W | 0 | 58 | 0.09375 | 32 | WCM89_Z17_1 | Tissue |
| 10 | 21901358 | C | T | MLLT10 | p.R163* | 0 | 46 | 0.069767 | 86 | WCM89_Z17_1 | Tissue |
| 10 | 35300798 | C | A | CUL2 | p.R695L | 0 | 41 | 0.125 | 48 | WCM89_Z17_1 | Tissue |
| 10 | 63195969 | C | T | TMEM26 | p.V77I | 0 | 112 | 0.054264 | 129 | WCM89_Z17_1 | Tissue |
| 11 | 1099262 | A | G | MUC2 | p.E2414G | 0 | 78 | 0.193548 | 62 | WCM89_Z17_1 | Tissue |
| 11 | 64573749 | C | T | MEN1 | p.R340Q | 0 | 80 | 0.076923 | 78 | WCM89_Z17_1 | Tissue |
| 11 | 64875352 | G | A | VPS51 | p.A195T | 0 | 80 | 0.101695 | 59 | WCM89_Z17_1 | Tissue |
| 11 | 65771217 | C | T | BANF1 | p.R82* | 0 | 95 | 0.058824 | 85 | WCM89_Z17_1 | Tissue |
| 11 | 76922284 | G | A | MYO7A | p.E2047K | 0 | 102 | 0.065217 | 92 | WCM89_Z17_1 | Tissue |
| 11 | 773579 | C | T | PDDC1 | p.D100N | 0 | 93 | 0.078125 | 64 | WCM89_Z17_1 | Tissue |
| 1 | 19439346 | G | A | UBR4 | p.R3825C | 0 | 91 | 0.090909 | 66 | WCM89_Z17_1 | Tissue |
| 12 | 133381505 | G | A | GOLGA3 | p.S465L | 0 | 87 | 0.064516 | 93 | WCM89_Z17_1 | Tissue |
| 1 | 228466650 | G | A | OBSCN | p.V2374M | 0 | 121 | 0.081081 | 74 | WCM89_Z17_1 | Tissue |
| 12 | 49086916 | C | T | CCNT1 | p.R694Q | 0 | 81 | 0.086957 | 46 | WCM89_Z17_1 | Tissue |
| 1 | 39322727 | C | T | RRAGC | p.E89K | 0 | 41 | 0.1 | 50 | WCM89_Z17_1 | Tissue |
| 14 | 102471007 | G | A | DYNC1H1 | p.R1679Q | 0 | 41 | 0.0625 | 48 | WCM89_Z17_1 | Tissue |
| 14 | 102807759 | C | T | ZNF839 | p.P560L | 0 | 131 | 0.15 | 60 | WCM89_Z17_1 | Tissue |
| 14 | 23994842 | G | A | ZFHX2 | p.R1437C | 0 | 65 | 0.081081 | 37 | WCM89_Z17_1 | Tissue |
| 15 | 93485061 | A | T | CHD2 | p.E234D | 0 | 52 | 0.289474 | 38 | WCM89_Z17_1 | Tissue |
| 16 | 15880573 | C | T | MYH11 | p.G183R | 0 | 52 | 0.066667 | 45 | WCM89_Z17_1 | Tissue |
| 16 | 30794825 | G | A | ZNF629 | p.S275L | 0 | 60 | 0.082192 | 73 | WCM89_Z17_1 | Tissue |
| 16 | 31335782 | G | A | ITGAM | p.A689T | 0 | 39 | 0.081081 | 37 | WCM89_Z17_1 | Tissue |
| 16 | 3828768 | C | T | CREBBP | p.R625H | 0 | 40 | 0.063158 | 95 | WCM89_Z17_1 | Tissue |
| 16 | 50325728 | G | A | ADCY7 | p.V153I | 0 | 77 | 0.09375 | 64 | WCM89_Z17_1 | Tissue |
| 17 | 40485737 | G | A | STAT3 | p.R335W | 0 | 52 | 0.114286 | 35 | WCM89_Z17_1 | Tissue |
| 17 | 42432404 | G | A | FAM171A2 | p.P393L | 0 | 99 | 0.090909 | 66 | WCM89_Z17_1 | Tissue |
| 17 | 48678431 | C | T | CACNA1G | p.R1271W | 0 | 91 | 0.075472 | 53 | WCM89_Z17_1 | Tissue |
| 17 | 60106978 | C | T | MED13 | p.E403K | 0 | 83 | 0.077922 | 77 | WCM89_Z17_1 | Tissue |
| 18 | 9535810 | G | A | RALBP1 | p.G615R | 0 | 64 | 0.084746 | 59 | WCM89_Z17_1 | Tissue |
| 19 | 47863315 | G | A | DHX34 | p.V455I | 0 | 172 | 0.051724 | 174 | WCM89_Z17_1 | Tissue |
| 19 | 55773980 | C | T | HSPBP1 | p.R359Q | 0 | 64 | 0.073171 | 41 | WCM89_Z17_1 | Tissue |
| 1 | 9795197 | G | A | CLSTN1 | p.P640L | 0 | 114 | 0.064516 | 62 | WCM89_Z17_1 | Tissue |
| 22 | 29737569 | C | T | AP1B1 | p.V573I | 0 | 122 | 0.08 | 75 | WCM89_Z17_1 | Tissue |
| 2 | 38570463 | G | A | ATL2 | p.R104C | 0 | 95 | 0.075949 | 79 | WCM89_Z17_1 | Tissue |
| 3 | 129233312 | G | A | IFT122 | p.R1023H | 0 | 81 | 0.057971 | 69 | WCM89_Z17_1 | Tissue |
| 3 | 136240108 | G | A | STAG1 | p.T208M | 0 | 124 | 0.054054 | 111 | WCM89_Z17_1 | Tissue |
| 3 | 49457751 | C | T | AMT | p.E122K | 0 | 98 | 0.0625 | 80 | WCM89_Z17_1 | Tissue |
| 4 | 151357970 | C | T | LRBA | p.R2287H | 0 | 42 | 0.06383 | 47 | WCM89_Z17_1 | Tissue |
| 4 | 66356247 | C | T | EPHA5 | p.R417Q | 0 | 31 | 0.058824 | 51 | WCM89_Z17_1 | Tissue |
| 5 | 114961344 | A | C | TMED7 | p.F49C | 0 | 113 | 0.23622 | 127 | WCM89_Z17_1 | Tissue |
| 5 | 131822527 | G | A | IRF1 | p.S125L | 0 | 66 | 0.068182 | 44 | WCM89_Z17_1 | Tissue |
| 5 | 16680190 | C | T | MYO10 | p.G1470R | 0 | 94 | 0.070423 | 71 | WCM89_Z17_1 | Tissue |
| 5 | 176700744 | C | T | NSD1 | p.R1861* | 0 | 141 | 0.056911 | 123 | WCM89_Z17_1 | Tissue |
| 5 | 66462662 | C | T | MAST4 | p.S2552L | 0 | 51 | 0.142857 | 35 | WCM89_Z17_1 | Tissue |
| 5 | 98234026 | A | C | CHD1 | p.F433L | 0 | 77 | 0.151515 | 132 | WCM89_Z17_1 | Tissue |
| 6 | 30866056 | G | A | DDR1 | p.R808H | 0 | 77 | 0.05814 | 86 | WCM89_Z17_1 | Tissue |
| 6 | 90660782 | G | A | BACH2 | p.T348M | 0 | 88 | 0.061728 | 81 | WCM89_Z17_1 | Tissue |
| 7 | 44294154 | C | T | CAMK2B | p.E110K | 0 | 82 | 0.067568 | 74 | WCM89_Z17_1 | Tissue |
| 8 | 17132424 | C | G | VPS37A | p.S200* | 0 | 104 | 0.263158 | 95 | WCM89_Z17_1 | Tissue |
| 9 | 122004459 | G | A | BRINP1 | p.R149C | 0 | 71 | 0.061856 | 97 | WCM89_Z17_1 | Tissue |
| 9 | 139273671 | G | A | SNAPC4 | p.R870W | 0 | 67 | 0.072727 | 55 | WCM89_Z17_1 | Tissue |
| X | 118786978 | C | T | 6-Sep | p.D123N | 0 | 61 | 0.072464 | 69 | WCM89_Z17_1 | Tissue |
| X | 129354372 | C | T | ZNF280C | p.R493H | 0 | 62 | 0.064103 | 78 | WCM89_Z17_1 | Tissue |
| X | 132160520 | G | A | USP26 | p.R577* | 0 | 56 | 0.076923 | 78 | WCM89_Z17_1 | Tissue |
| 10 | 45938974 | G | A | ALOX5 | p.R521H | 0 | 90 | 0.073529 | 68 | WCM89_ZLN1_1 | Tissue |
| 10 | 49448448 | C | T | FRMPD2 | p.E219K | 0 | 114 | 0.051471 | 136 | WCM89_ZLN1_1 | Tissue |
| 11 | 20070393 | C | T | NAV2 | p.P1364L | 0 | 47 | 0.069767 | 43 | WCM89_ZLN1_1 | Tissue |
| 11 | 20398139 | G | A | HTATIP2 | p.R106H | 0 | 119 | 0.063063 | 111 | WCM89_ZLN1_1 | Tissue |
| 1 | 146766162 | C | T | CHD1L | p.R860W | 0 | 56 | 0.0625 | 48 | WCM89_ZLN1_1 | Tissue |
| 11 | 62751802 | C | T | SLC22A6 | p.V121M | 0 | 104 | 0.054054 | 148 | WCM89_ZLN1_1 | Tissue |
| 1 | 33161326 | C | T | SYNC | p.V125M | 0 | 85 | 0.062016 | 129 | WCM89_ZLN1_1 | Tissue |
| 1 | 43748569 | G | A | C1orf210 | p.R77C | 0 | 46 | 0.085714 | 70 | WCM89_ZLN1_1 | Tissue |
| 1 | 46497194 | C | T | MAST2 | p.R1042C | 0 | 47 | 0.136364 | 44 | WCM89_ZLN1_1 | Tissue |
| 15 | 93485061 | A | T | CHD2 | p.E234D | 0 | 52 | 0.086957 | 46 | WCM89_ZLN1_1 | Tissue |
| 16 | 67293791 | G | A | SLC9A5 | p.R595H | 0 | 72 | 0.078947 | 76 | WCM89_ZLN1_1 | Tissue |
| 17 | 29559809 | C | T | NF1 | p.R1136W | 0 | 56 | 0.059322 | 118 | WCM89_ZLN1_1 | Tissue |
| 17 | 40485737 | G | A | STAT3 | p.R335W | 0 | 52 | 0.232558 | 43 | WCM89_ZLN1_1 | Tissue |
| 19 | 2434447 | G | A | LMNB2 | p.R330W | 0 | 43 | 0.096774 | 62 | WCM89_ZLN1_1 | Tissue |
| 19 | 45406310 | G | A | TOMM40 | p.V324M | 0 | 131 | 0.055944 | 143 | WCM89_ZLN1_1 | Tissue |
| 2 | 179588159 | C | T | TTN | p.R6906H | 0 | 196 | 0.159509 | 326 | WCM89_ZLN1_1 | Tissue |
| 3 | 119133444 | G | A | ARHGAP31 | p.E890K | 0 | 53 | 0.073171 | 41 | WCM89_ZLN1_1 | Tissue |
| 4 | 74864001 | G | A | CXCL5 | p.T55M | 0 | 53 | 0.114286 | 35 | WCM89_ZLN1_1 | Tissue |
| 4 | 83827705 | C | T | THAP9 | p.R169* | 0 | 79 | 0.16 | 125 | WCM89_ZLN1_1 | Tissue |
| 5 | 114961344 | A | C | TMED7 | p.F49C | 0 | 113 | 0.165775 | 187 | WCM89_ZLN1_1 | Tissue |
| 5 | 41911199 | G | A | C5orf51 | p.R135Q | 0 | 38 | 0.066667 | 45 | WCM89_ZLN1_1 | Tissue |
| 5 | 98234026 | A | C | CHD1 | p.F433L | 0 | 77 | 0.243243 | 148 | WCM89_ZLN1_1 | Tissue |
| 6 | 161139747 | C | T | PEG | p.R325C | 0 | 96 | 0.068182 | 44 | WCM89_ZLN1_1 | Tissue |
| 6 | 7889769 | G | A | TXNDC5 | p.R260C | 0 | 59 | 0.088235 | 68 | WCM89_ZLN1_1 | Tissue |
| 7 | 87436810 | G | A | RUNDC3B | p.R377Q | 0 | 46 | 0.079365 | 63 | WCM89_ZLN1_1 | Tissue |
| 8 | 146033632 | G | A | ZNF517 | p.R444Q | 0 | 44 | 0.116279 | 43 | WCM89_ZLN1_1 | Tissue |
| 8 | 1496891 | C | T | DLGAP2 | p.P11L | 0 | 100 | 0.102564 | 78 | WCM89_ZLN1_1 | Tissue |
| 8 | 17132424 | C | G | VPS37A | p.S200* | 0 | 104 | 0.120482 | 83 | WCM89_ZLN1_1 | Tissue |
| X | 128922020 | G | A | SASH3 | p.E43K | 0 | 82 | 0.050847 | 59 | WCM89_ZLN1_1 | Tissue |
| X | 51640096 | C | T | MAGED1 | p.R449C | 0 | 38 | 0.070175 | 57 | WCM89_ZLN1_1 | Tissue |
| 15 | 43748117 | A | C | TP53BP1 | p.S892A | 0 | 42 | 0.081081 | 74 | WCM9 | Plasma |
| 17 | 26100253 | G | T | NOS2 | p.T498N | 0 | 37 | 0.081081 | 37 | WCM9 | Plasma |
| 21 | 39817395 | C | T | ERG | p.W63* | 0 | 41 | 0.073529 | 68 | WCM9 | Plasma |
| 12 | 107386621 | G | A | CRY1 | p.Q569* | 0 | 55 | 0.134615 | 104 | WCM90 | Plasma |
| 19 | 38943561 | C | G | RYR1 | p.I449M | 0 | 33 | 0.289855 | 69 | WCM90 | Plasma |
| 19 | 51021681 | T | G | LRRC4B | p.D430A | 0 | 62 | 0.116505 | 103 | WCM90 | Plasma |
| 2 | 74041237 | C | A | C2orf78 | p.P244Q | 0 | 32 | 0.09589 | 73 | WCM90 | Plasma |
| 3 | 179605463 | G | T | PEX5L | p.A103D | 0 | 60 | 0.050847 | 118 | WCM90 | Plasma |
| 3 | 56771308 | G | A | ARHGEF3 | p.R316W | 0 | 31 | 0.150538 | 93 | WCM90 | Plasma |
| 4 | 71509275 | G | A | ENAM | p.R711K | 0 | 36 | 0.051282 | 78 | WCM90 | Plasma |
| 5 | 89791499 | G | C | POLR3G | p.E85Q | 0 | 59 | 0.188679 | 106 | WCM90 | Plasma |
| 6 | 47682185 | G | T | GPR115 | p.D402Y | 0 | 54 | 0.206522 | 92 | WCM90 | Plasma |
| 7 | 44116185 | C | T | POLM | p.R253Q | 0 | 36 | 0.133333 | 90 | WCM90 | Plasma |
| 7 | 82580050 | G | A | PCLO | p.T3285M | 0 | 34 | 0.255814 | 86 | WCM90 | Plasma |
| 10 | 126631331 | G | A | ZRANB1 | p.C90Y | 0 | 59 | 0.185185 | 54 | WCM90_Z1_1 | Tissue |
| 10 | 7822090 | C | T | KIN | p.R102Q | 0 | 146 | 0.233083 | 133 | WCM90_Z1_1 | Tissue |
| 11 | 100064331 | G | A | CNTN5 | p.W607* | 0 | 77 | 0.104167 | 48 | WCM90_Z1_1 | Tissue |
| 1 | 152733118 | C | T | KPRP | p.R352C | 0 | 65 | 0.421053 | 57 | WCM90_Z1_1 | Tissue |
| 11 | 65983717 | T | G | PACS1 | p.I263S | 0 | 54 | 0.333333 | 54 | WCM90_Z1_1 | Tissue |
| 12 | 107386621 | G | A | CRY1 | p.Q569* | 0 | 138 | 0.271186 | 177 | WCM90_Z1_1 | Tissue |
| 15 | 75120439 | C | T | CPLX3 | p.R77* | 0 | 192 | 0.207254 | 193 | WCM90_Z1_1 | Tissue |
| 16 | 56500800 | C | G | OGFOD1 | p.P193R | 0 | 31 | 0.285714 | 42 | WCM90_Z1_1 | Tissue |
| 16 | 57794796 | G | A | KIFC3 | p.R692W | 0 | 37 | 0.166667 | 72 | WCM90_Z1_1 | Tissue |
| 16 | 87678040 | G | A | JPH3 | p.G187S | 0 | 47 | 0.140351 | 114 | WCM90_Z1_1 | Tissue |
| 17 | 47696426 | A | T | SPOP | p.F133I | 0 | 123 | 0.37931 | 116 | WCM90_Z1_1 | Tissue |
| 2 | 187366061 | T | A | ZC3H15 | p.D117E | 0 | 44 | 0.204082 | 49 | WCM90_Z1_1 | Tissue |
| 22 | 21355014 | C | T | THAP7 | p.R100H | 0 | 39 | 0.06 | 50 | WCM90_Z1_1 | Tissue |
| 22 | 24237638 | A | G | AP000350.4 | p.V56A | 0 | 53 | 0.4 | 60 | WCM90_Z1_1 | Tissue |
| 2 | 74041237 | C | A | C2orf78 | p.P244Q | 0 | 34 | 0.2 | 50 | WCM90_Z1_1 | Tissue |
| 3 | 134911606 | C | T | EPHB1 | p.R691W | 0 | 156 | 0.302469 | 162 | WCM90_Z1_1 | Tissue |
| 3 | 179605463 | G | T | PEX5L | p.A103D | 0 | 57 | 0.193182 | 88 | WCM90_Z1_1 | Tissue |
| 3 | 56771308 | G | A | ARHGEF3 | p.R316W | 0 | 43 | 0.375 | 40 | WCM90_Z1_1 | Tissue |
| 4 | 114436287 | G | T | CAMK2D | p.L253I | 0 | 38 | 0.071429 | 42 | WCM90_Z1_1 | Tissue |
| 4 | 71509275 | G | A | ENAM | p.R711K | 0 | 84 | 0.091667 | 120 | WCM90_Z1_1 | Tissue |
| 5 | 112175687 | G | A | APC | p.G1466R | 0 | 137 | 0.2 | 80 | WCM90_Z1_1 | Tissue |
| 5 | 149357479 | G | T | SLC26A2 | p.M88I | 0 | 53 | 0.405797 | 69 | WCM90_Z1_1 | Tissue |
| 5 | 156916172 | T | G | ADAM19 | p.N755H | 0 | 79 | 0.416667 | 96 | WCM90_Z1_1 | Tissue |
| 5 | 169805776 | C | A | KCNMB1 | p.G170C | 0 | 42 | 0.085714 | 35 | WCM90_Z1_1 | Tissue |
| 6 | 12124055 | G | A | HIVEP1 | p.E1343K | 0 | 67 | 0.076923 | 52 | WCM90_Z1_1 | Tissue |
| 6 | 47682185 | G | T | GPR115 | p.D402Y | 0.005587 | 179 | 0.769784 | 139 | WCM90_Z1_1 | Tissue |
| 7 | 24905026 | C | A | OSBPL3 | p.C203F | 0 | 39 | 0.06383 | 47 | WCM90_Z1_1 | Tissue |
| 7 | 44116185 | C | T | POLM | p.R253Q | 0 | 112 | 0.333333 | 159 | WCM90_Z1_1 | Tissue |
| 1 | 152733118 | C | T | KPRP | p.R352C | 0 | 65 | 0.101449 | 69 | WCM90_Z19_1 | Tissue |
| 11 | 55433205 | C | T | OR4C6 | p.T188M | 0 | 68 | 0.166667 | 60 | WCM90_Z19_1 | Tissue |
| 11 | 65983717 | T | G | PACS1 | p.I263S | 0 | 54 | 0.237288 | 59 | WCM90_Z19_1 | Tissue |
| 12 | 107386621 | G | A | CRY1 | p.Q569* | 0 | 138 | 0.148936 | 141 | WCM90_Z19_1 | Tissue |
| 12 | 50749478 | G | T | FAM186A | p.D379E | 0 | 39 | 0.085714 | 35 | WCM90_Z19_1 | Tissue |
| 14 | 70855215 | C | T | SYNJ2BP | p.R58Q | 0 | 103 | 0.093023 | 86 | WCM90_Z19_1 | Tissue |
| 15 | 90026426 | C | A | RHCG | p.V132L | 0 | 34 | 0.171429 | 35 | WCM90_Z19_1 | Tissue |
| 16 | 56500800 | C | G | OGFOD1 | p.P193R | 0 | 31 | 0.090909 | 33 | WCM90_Z19_1 | Tissue |
| 17 | 47696426 | A | T | SPOP | p.F133I | 0 | 123 | 0.12963 | 108 | WCM90_Z19_1 | Tissue |
| 22 | 24237638 | A | G | AP000350.4 | p.V56A | 0 | 53 | 0.126984 | 63 | WCM90_Z19_1 | Tissue |
| 22 | 30415490 | C | G | MTMR3 | p.Y614* | 0 | 154 | 0.10241 | 166 | WCM90_Z19_1 | Tissue |
| 3 | 134911606 | C | T | EPHB1 | p.R691W | 0 | 156 | 0.155556 | 135 | WCM90_Z19_1 | Tissue |
| 3 | 39374147 | G | T | CCR8 | p.V109L | 0 | 46 | 0.0625 | 48 | WCM90_Z19_1 | Tissue |
| 5 | 149357479 | G | T | SLC26A2 | p.M88I | 0 | 53 | 0.101124 | 89 | WCM90_Z19_1 | Tissue |
| 5 | 156916172 | T | G | ADAM19 | p.N755H | 0 | 79 | 0.159091 | 132 | WCM90_Z19_1 | Tissue |
| 5 | 89791499 | G | C | POLR3G | p.E85Q | 0 | 42 | 0.1 | 40 | WCM90_Z19_1 | Tissue |
| 6 | 47682185 | G | T | GPR115 | p.D402Y | 0.005587 | 179 | 0.186207 | 145 | WCM90_Z19_1 | Tissue |
| 7 | 137586116 | G | C | CREB3L2 | p.L343V | 0 | 162 | 0.174497 | 149 | WCM90_Z19_1 | Tissue |
| 1 | 152733118 | C | T | KPRP | p.R352C | 0 | 65 | 0.588235 | 51 | WCM90_Z21_1 | Tissue |
| 11 | 65983717 | T | G | PACS1 | p.I263S | 0 | 54 | 0.461538 | 52 | WCM90_Z21_1 | Tissue |
| 12 | 107386621 | G | A | CRY1 | p.Q569* | 0 | 138 | 0.329268 | 246 | WCM90_Z21_1 | Tissue |
| 13 | 113485800 | C | A | ATP11A | p.Q445K | 0 | 105 | 0.056604 | 53 | WCM90_Z21_1 | Tissue |
| 1 | 6186793 | C | T | CHD5 | p.R1306Q | 0 | 51 | 0.066667 | 45 | WCM90_Z21_1 | Tissue |
| 16 | 87678040 | G | A | JPH3 | p.G187S | 0 | 47 | 0.181818 | 77 | WCM90_Z21_1 | Tissue |
| 17 | 47696426 | A | T | SPOP | p.F133I | 0 | 123 | 0.41129 | 124 | WCM90_Z21_1 | Tissue |
| 19 | 1272015 | C | T | CIRBP | p.S156L | 0 | 37 | 0.083333 | 48 | WCM90_Z21_1 | Tissue |
| 19 | 34900207 | G | T | PDCD2L | p.A160S | 0 | 88 | 0.061856 | 97 | WCM90_Z21_1 | Tissue |
| 20 | 44586302 | C | A | ZNF335 | p.A789S | 0 | 111 | 0.05641 | 195 | WCM90_Z21_1 | Tissue |
| 2 | 187366061 | T | A | ZC3H15 | p.D117E | 0 | 44 | 0.142857 | 70 | WCM90_Z21_1 | Tissue |
| 22 | 24237638 | A | G | AP000350.4 | p.V56A | 0 | 53 | 0.303571 | 56 | WCM90_Z21_1 | Tissue |
| 2 | 61258865 | G | T | PEX13 | p.S135I | 0 | 152 | 0.078947 | 76 | WCM90_Z21_1 | Tissue |
| 3 | 134911606 | C | T | EPHB1 | p.R691W | 0 | 156 | 0.461538 | 169 | WCM90_Z21_1 | Tissue |
| 3 | 179605463 | G | T | PEX5L | p.A103D | 0 | 57 | 0.373333 | 75 | WCM90_Z21_1 | Tissue |
| 3 | 56771308 | G | A | ARHGEF3 | p.R316W | 0 | 43 | 0.44186 | 43 | WCM90_Z21_1 | Tissue |
| 4 | 71509275 | G | A | ENAM | p.R711K | 0 | 84 | 0.081081 | 74 | WCM90_Z21_1 | Tissue |
| 5 | 112175687 | G | A | APC | p.G1466R | 0 | 137 | 0.425532 | 47 | WCM90_Z21_1 | Tissue |
| 5 | 149357479 | G | T | SLC26A2 | p.M88I | 0 | 53 | 0.243243 | 74 | WCM90_Z21_1 | Tissue |
| 5 | 156916172 | T | G | ADAM19 | p.N755H | 0 | 79 | 0.594937 | 79 | WCM90_Z21_1 | Tissue |
| 5 | 89791499 | G | C | POLR3G | p.E85Q | 0 | 42 | 0.772727 | 44 | WCM90_Z21_1 | Tissue |
| 6 | 47682185 | G | T | GPR115 | p.D402Y | 0.005587 | 179 | 0.818182 | 121 | WCM90_Z21_1 | Tissue |
| 6 | 87966658 | G | T | ZNF292 | p.G1104V | 0 | 120 | 0.053571 | 56 | WCM90_Z21_1 | Tissue |
| 7 | 44116185 | C | T | POLM | p.R253Q | 0 | 112 | 0.292994 | 157 | WCM90_Z21_1 | Tissue |
| 8 | 11058363 | G | T | XKR6 | p.F162L | 0 | 37 | 0.09375 | 32 | WCM90_Z21_1 | Tissue |
| 1 | 152733118 | C | T | KPRP | p.R352C | 0 | 65 | 0.150685 | 73 | WCM90_Z25_1 | Tissue |
| 12 | 107386621 | G | A | CRY1 | p.Q569* | 0 | 138 | 0.155405 | 148 | WCM90_Z25_1 | Tissue |
| 17 | 47696426 | A | T | SPOP | p.F133I | 0 | 123 | 0.154472 | 123 | WCM90_Z25_1 | Tissue |
| 17 | 63189706 | G | T | RGS9 | p.M264I | 0 | 42 | 0.078947 | 38 | WCM90_Z25_1 | Tissue |
| 2 | 179484486 | C | G | TTN | p.V13879L | 0 | 52 | 0.121212 | 33 | WCM90_Z25_1 | Tissue |
| 3 | 134911606 | C | T | EPHB1 | p.R691W | 0 | 156 | 0.147059 | 170 | WCM90_Z25_1 | Tissue |
| 3 | 20212228 | G | A | SGOL1 | p.R518C | 0 | 147 | 0.116883 | 154 | WCM90_Z25_1 | Tissue |
| 3 | 56771308 | G | A | ARHGEF3 | p.R316W | 0 | 43 | 0.153846 | 52 | WCM90_Z25_1 | Tissue |
| 5 | 112175687 | G | A | APC | p.G1466R | 0 | 137 | 0.059322 | 118 | WCM90_Z25_1 | Tissue |
| 5 | 149357479 | G | T | SLC26A2 | p.M88I | 0 | 53 | 0.166667 | 66 | WCM90_Z25_1 | Tissue |
| 5 | 156916172 | T | G | ADAM19 | p.N755H | 0 | 79 | 0.145455 | 110 | WCM90_Z25_1 | Tissue |
| 5 | 89791499 | G | C | POLR3G | p.E85Q | 0 | 42 | 0.131579 | 38 | WCM90_Z25_1 | Tissue |
| 6 | 47682185 | G | T | GPR115 | p.D402Y | 0.005587 | 179 | 0.188525 | 122 | WCM90_Z25_1 | Tissue |
| 7 | 137586116 | G | C | CREB3L2 | p.L343V | 0 | 162 | 0.163043 | 184 | WCM90_Z25_1 | Tissue |
| 10 | 7822090 | C | T | KIN | p.R102Q | 0 | 146 | 0.158879 | 107 | WCM90_Z29_1 | Tissue |
| 1 | 152733118 | C | T | KPRP | p.R352C | 0 | 65 | 0.285714 | 63 | WCM90_Z29_1 | Tissue |
| 11 | 65983717 | T | G | PACS1 | p.I263S | 0 | 54 | 0.220339 | 59 | WCM90_Z29_1 | Tissue |
| 12 | 107386621 | G | A | CRY1 | p.Q569* | 0 | 138 | 0.235772 | 123 | WCM90_Z29_1 | Tissue |
| 15 | 75120439 | C | T | CPLX3 | p.R77* | 0 | 192 | 0.131868 | 182 | WCM90_Z29_1 | Tissue |
| 17 | 47696426 | A | T | SPOP | p.F133I | 0 | 123 | 0.278846 | 104 | WCM90_Z29_1 | Tissue |
| 19 | 51021681 | T | G | LRRC4B | p.D430A | 0 | 41 | 0.12 | 100 | WCM90_Z29_1 | Tissue |
| 22 | 24237638 | A | G | AP000350.4 | p.V56A | 0 | 53 | 0.152174 | 46 | WCM90_Z29_1 | Tissue |
| 2 | 74041237 | C | A | C2orf78 | p.P244Q | 0 | 34 | 0.125 | 32 | WCM90_Z29_1 | Tissue |
| 3 | 134911606 | C | T | EPHB1 | p.R691W | 0 | 156 | 0.131944 | 144 | WCM90_Z29_1 | Tissue |
| 3 | 179605463 | G | T | PEX5L | p.A103D | 0 | 57 | 0.15873 | 63 | WCM90_Z29_1 | Tissue |
| 3 | 56771308 | G | A | ARHGEF3 | p.R316W | 0 | 43 | 0.25 | 32 | WCM90_Z29_1 | Tissue |
| 5 | 112175687 | G | A | APC | p.G1466R | 0 | 137 | 0.177778 | 90 | WCM90_Z29_1 | Tissue |
| 5 | 149357479 | G | T | SLC26A2 | p.M88I | 0 | 53 | 0.123077 | 65 | WCM90_Z29_1 | Tissue |
| 5 | 156916172 | T | G | ADAM19 | p.N755H | 0 | 79 | 0.223404 | 94 | WCM90_Z29_1 | Tissue |
| 5 | 89791499 | G | C | POLR3G | p.E85Q | 0 | 42 | 0.236842 | 38 | WCM90_Z29_1 | Tissue |
| 6 | 47682185 | G | T | GPR115 | p.D402Y | 0.005587 | 179 | 0.325 | 120 | WCM90_Z29_1 | Tissue |
| 11 | 124189582 | G | T | OR8D2 | p.S171Y | 0 | 88 | 0.652778 | 72 | WCM91_Z1_1 | Tissue |
| 1 | 205211032 | C | T | TMCC2 | p.Q203* | 0 | 50 | 0.3 | 60 | WCM91_Z1_1 | Tissue |
| 12 | 110765737 | C | T | ATP2A2 | p.P337L | 0 | 117 | 0.436782 | 174 | WCM91_Z1_1 | Tissue |
| 1 | 232596637 | G | A | SIPA1L2 | p.R1031* | 0 | 193 | 0.587814 | 279 | WCM91_Z1_1 | Tissue |
| 1 | 248845130 | C | A | OR14I1 | p.G159V | 0 | 131 | 0.588235 | 204 | WCM91_Z1_1 | Tissue |
| 12 | 95914845 | C | T | USP44 | p.A623T | 0 | 199 | 0.117347 | 196 | WCM91_Z1_1 | Tissue |
| 13 | 111944474 | G | A | ARHGEF7 | p.G736D | 0 | 44 | 0.71875 | 32 | WCM91_Z1_1 | Tissue |
| 14 | 50459539 | C | T | C14orf182 | p.V87I | 0 | 36 | 0.170732 | 41 | WCM91_Z1_1 | Tissue |
| 15 | 45814220 | T | G | SLC30A4 | p.K111N | 0 | 88 | 0.1 | 70 | WCM91_Z1_1 | Tissue |
| 1 | 78401682 | G | A | NEXN | p.A476T | 0 | 44 | 0.089552 | 67 | WCM91_Z1_1 | Tissue |
| 19 | 13928586 | G | A | ZSWIM4 | p.G457R | 0 | 45 | 0.263158 | 57 | WCM91_Z1_1 | Tissue |
| 19 | 39380581 | G | A | SIRT2 | p.R97C | 0 | 104 | 0.181102 | 127 | WCM91_Z1_1 | Tissue |
| 19 | 44610939 | G | T | ZNF224 | p.C209F | 0 | 128 | 0.404255 | 141 | WCM91_Z1_1 | Tissue |
| 19 | 44833294 | G | A | ZNF112 | p.P345L | 0 | 80 | 0.37931 | 116 | WCM91_Z1_1 | Tissue |
| 19 | 50156923 | C | G | SCAF1 | p.P1093A | 0 | 65 | 0.112676 | 71 | WCM91_Z1_1 | Tissue |
| 2 | 10140761 | T | G | GRHL1 | p.S595A | 0 | 81 | 0.16129 | 93 | WCM91_Z1_1 | Tissue |
| 21 | 45999762 | C | T | KRTAP10-5 | p.A232T | 0 | 124 | 0.585714 | 140 | WCM91_Z1_1 | Tissue |
| 2 | 160239129 | C | T | BAZ2B | p.E1316K | 0 | 114 | 0.192308 | 104 | WCM91_Z1_1 | Tissue |
| 2 | 161025757 | A | G | ITGB6 | p.F328S | 0 | 60 | 0.133333 | 60 | WCM91_Z1_1 | Tissue |
| 2 | 80782980 | A | G | CTNNA2 | p.E568G | 0 | 149 | 0.30303 | 198 | WCM91_Z1_1 | Tissue |
| 3 | 164905781 | G | T | SLITRK3 | p.H946Q | 0 | 94 | 0.484536 | 97 | WCM91_Z1_1 | Tissue |
| 6 | 10756717 | A | C | TMEM14B | p.K104T | 0 | 179 | 0.2 | 195 | WCM91_Z1_1 | Tissue |
| 6 | 39688562 | T | G | KIF6 | p.K31T | 0 | 36 | 0.150943 | 53 | WCM91_Z1_1 | Tissue |
| 7 | 107417117 | T | G | SLC26A3 | p.T517P | 0 | 38 | 0.181818 | 44 | WCM91_Z1_1 | Tissue |
| X | 40464879 | A | T | ATP6AP2 | p.M309L | 0 | 42 | 0.597938 | 97 | WCM91_Z1_1 | Tissue |
| 11 | 10503668 | G | A | AMPD3 | p.R171Q | 0 | 184 | 0.257862 | 159 | WCM9_X1_1 | Tissue |
| 11 | 75438598 | C | T | MOGAT2 | p.S130L | 0 | 131 | 0.066667 | 120 | WCM9_X1_1 | Tissue |
| 12 | 18872552 | G | T | PLCZ1 | p.Q128K | 0 | 37 | 0.238806 | 67 | WCM9_X1_1 | Tissue |
| 1 | 27332698 | G | A | FAM46B | p.R339C | 0 | 85 | 0.134328 | 67 | WCM9_X1_1 | Tissue |
| 14 | 21360033 | G | A | RNASE3 | p.R63H | 0 | 113 | 0.181818 | 110 | WCM9_X1_1 | Tissue |
| 14 | 35872358 | T | C | NFKBIA | p.N182S | 0 | 52 | 0.28 | 100 | WCM9_X1_1 | Tissue |
| 14 | 75513676 | G | A | MLH3 | p.R895C | 0 | 77 | 0.067308 | 104 | WCM9_X1_1 | Tissue |
| 15 | 81585194 | G | A | IL16 | p.R573Q | 0 | 37 | 0.111111 | 36 | WCM9_X1_1 | Tissue |
| 16 | 18828765 | G | T | SMG1 | p.H3308N | 0 | 68 | 0.157895 | 76 | WCM9_X1_1 | Tissue |
| 16 | 23721844 | C | T | ERN2 | p.V76I | 0 | 41 | 0.283333 | 60 | WCM9_X1_1 | Tissue |
| 17 | 73239148 | G | A | GGA3 | p.S175F | 0 | 83 | 0.071429 | 70 | WCM9_X1_1 | Tissue |
| 19 | 17396343 | C | T | ANKLE1 | p.R494W | 0 | 113 | 0.311475 | 122 | WCM9_X1_1 | Tissue |
| 20 | 13260377 | C | T | ISM1 | p.R159W | 0 | 45 | 0.241935 | 62 | WCM9_X1_1 | Tissue |
| 3 | 50154781 | G | A | RBM5 | p.R764Q | 0 | 69 | 0.238806 | 67 | WCM9_X1_1 | Tissue |
| 5 | 140181632 | G | A | PCDHA3 | p.D284N | 0 | 90 | 0.304688 | 128 | WCM9_X1_1 | Tissue |
| 5 | 154278847 | G | C | GEMIN5 | p.A1013G | 0 | 86 | 0.158879 | 107 | WCM9_X1_1 | Tissue |
| 1 | 100214293 | C | T | FRRS1 | p.C11Y | 0 | 63 | 0.081081 | 37 | WCM9_Z3-1 | Tissue |
| 11 | 10503668 | G | A | AMPD3 | p.R171Q | 0 | 184 | 0.261538 | 65 | WCM9_Z3-1 | Tissue |
| 11 | 119181152 | G | T | MCAM | p.S606R | 0 | 143 | 0.096386 | 83 | WCM9_Z3-1 | Tissue |
| 12 | 104373677 | C | A | TDG | p.P79T | 0 | 77 | 0.257143 | 35 | WCM9_Z3-1 | Tissue |
| 12 | 13716863 | G | C | GRIN2B | p.D1103E | 0 | 32 | 0.143939 | 264 | WCM9_Z3-1 | Tissue |
| 1 | 27332698 | G | A | FAM46B | p.R339C | 0 | 85 | 0.230769 | 65 | WCM9_Z3-1 | Tissue |
| 14 | 21360033 | G | A | RNASE3 | p.R63H | 0 | 113 | 0.268657 | 134 | WCM9_Z3-1 | Tissue |
| 15 | 23686113 | C | G | GOLGA6L2 | p.Q503H | 0 | 36 | 0.148456 | 842 | WCM9_Z3-1 | Tissue |
| 16 | 3066004 | G | A | CLDN6 | p.Q7* | 0 | 162 | 0.052632 | 133 | WCM9_Z3-1 | Tissue |
| 16 | 71660273 | C | A | MARVELD3 | p.S47R | 0 | 32 | 0.058824 | 51 | WCM9_Z3-1 | Tissue |
| 17 | 1686445 | C | A | SMYD4 | p.W715C | 0 | 200 | 0.1 | 50 | WCM9_Z3-1 | Tissue |
| 17 | 39261693 | A | T | KRTAP4-9 | p.D18V | 0 | 39 | 0.083333 | 48 | WCM9_Z3-1 | Tissue |
| 17 | 80333060 | C | T | UTS2R | p.P287L | 0 | 83 | 0.232143 | 56 | WCM9_Z3-1 | Tissue |
| 19 | 17396343 | C | T | ANKLE1 | p.R494W | 0 | 113 | 0.292308 | 65 | WCM9_Z3-1 | Tissue |
| 19 | 44057183 | G | A | XRCC1 | p.A221V | 0 | 78 | 0.06 | 50 | WCM9_Z3-1 | Tissue |
| 19 | 48519277 | G | T | ELSPBP1 | p.W112C | 0 | 33 | 0.061224 | 49 | WCM9_Z3-1 | Tissue |
| 19 | 49969496 | C | T | ALDH16A1 | p.R632W | 0 | 107 | 0.2 | 45 | WCM9_Z3-1 | Tissue |
| 19 | 51826981 | G | A | IGLON5 | p.G75D | 0 | 46 | 0.068182 | 44 | WCM9_Z3-1 | Tissue |
| 20 | 13260377 | C | T | ISM1 | p.R159W | 0 | 45 | 0.25974 | 77 | WCM9_Z3-1 | Tissue |
| 2 | 230456343 | G | A | DNER | p.P180S | 0 | 94 | 0.202632 | 380 | WCM9_Z3-1 | Tissue |
| 5 | 149511596 | G | A | PDGFRB | p.R397W | 0 | 65 | 0.21875 | 128 | WCM9_Z3-1 | Tissue |
| 5 | 154278847 | G | C | GEMIN5 | p.A1013G | 0 | 86 | 0.307692 | 104 | WCM9_Z3-1 | Tissue |
| 7 | 107708565 | C | T | LAMB4 | p.C781Y | 0 | 86 | 0.072115 | 208 | WCM9_Z3-1 | Tissue |
| 7 | 76828504 | G | T | FGL2 | p.R203S | 0 | 39 | 0.180147 | 272 | WCM9_Z3-1 | Tissue |
| 8 | 116631420 | C | T | TRPS1 | p.G289D | 0 | 110 | 0.073171 | 82 | WCM9_Z3-1 | Tissue |
| 9 | 34725069 | T | A | FAM205A | p.E723V | 0 | 55 | 0.1875 | 48 | WCM9_Z3-1 | Tissue |
| X | 73962078 | G | A | KIAA2022 | p.R772C | 0 | 33 | 0.119658 | 117 | WCM9_Z3-1 | Tissue |

*denotes a nonsense mutation

(vii) Similarity Measure Based on Copy Number Profiles

For each patient, the somatic copy number aberration (SCNA) profiles of tumor tissues and cfDNA samples were compared (Table 3). Comparison was performed using an ad-hoc notion of similarity, which exploits tumor content corrected SCNA profiles (FIG. 1B) and measures the percentage of concordant aberration signal. Specifically, given a sample P and a sample T, the algorithm computes the following steps:

1. SCNA profile signal of sample P is centered around the P mean signal;

2. SCNA profile signal of sample T is centered around the T mean signal;

3. SCNA signals of P and T samples are synchronized around their median difference to avoid the presence of systematic signal shifts due to technical or processing bias. Specifically, SCNAs signal P is normalized as P=P −median(P−T);

4. a measure of loss similarity Sₗₒₛₛ is obtained by measuring the fraction of genes having signal below the detection threshold −THR in both P and T profiles over the total number of genes having a signal below the detection threshold −THR in at least one of the two samples;

5. a measure of gain similarity S_{gain} is obtained by measuring the fraction of genes having signal above the detection threshold THR in both P and T profiles over the total number of genes having a signal above the detection threshold THR in at least one of the two samples;

6. A global similarity measure is obtained as S=(Sₗₒₛₛ+S_{gain})/2.

A model with 19,027 genes was here considered along with a detection threshold THR equal to 0.3.

f. Similarity Measure Based on SNV Profiles

For each patient and each plasma and tumor tissue sample's pair private and shared somatic SNVs were then compiled. For each patient, an inclusion matrix considering all plasma-tissue and tissue-tissue pairs was computed, including non-synonymous SNVs only. Specifically, for each pair (P1, P2) of samples the inclusion matrix reports both the fraction of mutations detected in P1 that are also detected in P2 and the fraction of mutations detected in P2 that are also detected in P1.

g. Estimation of Clonality Divergence

Non-discretized estimations of allele-specific copy number analysis (Table 3) were used to calculate the Clonality Divergence Index. For each sample and each genomic segment, the Euclidean difference between the estimated raw allele-specific copy number and the closest expected clonal allele-specific copy number was assessed. The average of the minimum distances is the Clonality Divergence Index. Formally, considering a sample P and segment S and its estimated allele-specific copy number coordinates (cnA, cnB), in a Euclidean space with can and cnB real numbers, the value was compute:

$${D}_{S} = {min}_{e ∈ C} \overline{ep}$$

Where e̅p̅ is the Euclidian distance of p=(cnA, cnB) and a point in C={(a, b)|a, b∈M} with M equal to the least integer greater than or equal to the maximum raw cnA or cnB estimated across all P segments. The Clonality Divergence index of P is the mean of all Ds computed for all segments S of P. A sample with all and only segments with perfect integer (cnA, cnB) would have a Clonality Divergence Index equal to zero.

h. Whole Genome Bisulfite Sequencing (WGBS) Data Generation and Processing

5 ng of cfDNA and 100 ng of germline DNA were sonicated using a Covaris S220 to approximately 180-220 bp (Covaris, Woburn, Mass.) and bisulfite converted using EZ DNA Methylation-Gold Kit (cat # D5005, Zymo Research Corporation, Irvine, Calif.). The single-stranded DNA obtained was processed for library construction using the Accel-NGS@Methyl-seq DNA Library kit (cat #36024) as per manufacturer instructions (Swift BioSciences, Ann Arbor, Mich.). Briefly, truncated adapter sequences were incorporated to the single-stranded DNA in a template-independent reaction through sequential steps using the Adaptase™ module. DNA was then enriched using PCR with primers compatible with Illumina sequencing, 9 cycles for cfDNA and 6 cycles for genomic DNA. The libraries were clustered at 12 pM on a pair end read flow cell and sequenced for 125 cycles on Illumina HiSeq 2500 or 4000. Primary processing of sequencing images was done using Illumina's Real Time Analysis software (RTA). CASAVA 1.8.2 software was then used to demultiplex samples and generate raw reads and respective quality scores. The WGBS raw data was quality filtered and adapter trimmed using Flexbar with parameters -ao 6, -m 21, -at 2. Reads were aligned to unmasked Human genome build GRCh37/hg19 and methylation calls were generated with Bismark (Krueger and Andrews (2011) Bioinformatics 27:1571-1572) as described in the data analysis section of the Epigenomics Core in-house bisulfite sequencing analysis pipeline (Garrett-Bakelman et al. (2015) J Vis Exp:e52246). The average conversion rate in WGBS samples is 99.6%, the average CpG coverage is 14.3 and the average mapping efficiency is 76% (Table 5). For plasma related analysis, Only CpG sites covered by at least 10 reads and read in at least 10% of the study samples were considered for downstream analysis. For each sample, the percentage of methylation per site (beta value) was computed. ctDNA fraction was estimated by PAMES, using 10 hyper-methylated prostate specific CpG islands (Benelli, Romagnoli, and Demichelis (2018) Bioinformatics 34:1642-1649). CpG wise differential methylation analysis (CRPC-NE versus CRPC-Adeno) was performed by Area Under the Curve (AUC) analysis. Hyper- and hypo-methylated sites were identified as those sites demonstrating AUC=1 and AUC=0, respectively. Genomic annotation of methylation sites was performed by the tool annotatePeaks included in the HOMER package16 (Table 6).

TABLE 5
Aligner sequencing statistics for WGBS samples

| | | Mean | avg. | mapping | |
|---|---|---|---|---|---|
| | | CpG | conversion | efficiency | |
| Sample | Type | cov. | rate (%) | (%) | Mapped_reads |
| WCM0 | Plasma | 13.64 | 99.62 | 76.90% | 767980146 |
| WCM0 | Germline | 13.64 | 99.62 | 76.90% | 583747822 |
| WCM14 | Plasma | 14.06 | 99.62 | 76.00% | 679594352 |
| WCM14 | Germline | 14.06 | 99.62 | 76.00% | 633594624 |
| WCM78 | Plasma | 15.29 | 99.43 | 80.20% | 615261868 |
| WCM78 | Germline | 13.4 | 99.63 | 75.50% | 543578974 |
| WCM90 | Plasma | 14.47 | 99.47 | 79.10% | 626545886 |
| WCM90 | Germline | 14.18 | 99.62 | 76.80% | 658399870 |
| WCM119 | Plasma | 14.58 | 99.52 | 78.40% | 669516040 |
| WCM119 | Germline | 14.17 | 99.63 | 75.90% | 634191076 |
| WCM154 | Plasma | 14.44 | 99.57 | 79.50% | 638284428 |
| WCM154 | Germline | 13.66 | 99.63 | 75.30% | 556590598 |
| WCM155 | Plasma | 15.08 | 99.6 | 72.90% | 813407242 |
| WCM155 | Germline | 14.97 | 99.71 | 71.20% | 748223332 |
| WCM161 | Plasma | 15.12 | 99.52 | 71.00% | 714276482 |
| WCM161 | Germline | 14.9 | 99.68 | 72.40% | 758044212 |
| WCM163 | Plasma | 14.51 | 99.55 | 78.50% | 626607384 |
| WCM163 | Germline | 14.05 | 99.62 | 76.00% | 624074488 |
| WCM207 | Plasma | 14.39 | 99.56 | 79.60% | 644873354 |
| WCM207 | Germline | 14.22 | 99.58 | 76.20% | 642641686 |
| WCM677 | Plasma | 14 | 99.54 | 78.70% | 555277164 |
| WCM677 | Germline | 13.94 | 99.6 | 76.30% | 616407358 |

TABLE 6
Methylation sites included in the NEPC feature score

| | | | Methylation | | | |
|---|---|---|---|---|---|---|
| | | | status | | | Nearest |
| | Start | End | (NE vs | | Distance | Promoter |
| chr | (hg19) | (hg19) | Adeno) | Annotation | to TSS | ID |
| chr1 | 241206098 | 241206097 | hyper | intron | −89475 | NR_036069 |
| | | | | (NM_001282773, | | |
| | | | | intron 3 of 15) | | |
| chr18 | 47258089 | 47258088 | hyper | Intergenic | 82163 | NM_006111 |
| chr21 | 32546326 | 32546325 | hyper | intron | −135530 | NM_001099219 |
| | | | | (NM_003253, | | |
| | | | | intron 16 of 28) | | |
| chr5 | 1548487 | 1548486 | hyper | Intergenic | −24410 | NM_024830 |
| chr5 | 71146894 | 71146893 | hyper | Intergenic | 131903 | NM_004291 |
| chr5 | 142194123 | 142194122 | hyper | intron | 43830 | NM_015071 |
| | | | | (NM_015071, | | |
| | | | | intron 1 of 22) | | |
| chr7 | 7799018 | 7799017 | hyper | intron | −40779 | NM_002947 |
| | | | | (NM_001302350, | | |
| | | | | intron 3 of 4) | | |
| chr7 | 127841628 | 127841627 | hyper | Intergenic | −6298 | NR_029596 |
| chr8 | 53499560 | 53499559 | hyper | Intergenic | −21538 | NM_207413 |
| chr8 | 73479990 | 73479989 | hyper | exon | 30363 | NM_004770 |
| | | | | (NM_004770, | | |
| | | | | exon 2 of 3) | | |
| chr8 | 73849054 | 73849053 | hyper | exon | −72044 | NM_003218 |
| | | | | (NM_004770, | | |
| | | | | exon 3 of 3) | | |
| chr8 | 140727998 | 140727997 | hyper | Intergenic | −12698 | NR_104210 |
| chr8 | 141994084 | 141994083 | hyper | intron | 17329 | NM_001199649 |
| | | | | (NM_153831, | | |
| | | | | intron 1 of 31) | | |
| chr8 | 142367954 | 142367953 | hyper | exon | 9412 | NM_005293 |
| | | | | (NM_005293, | | |
| | | | | exon 2 of 2) | | |
| chr8 | 143395944 | 143395943 | hyper | intron | 88667 | NM_145003 |
| | | | | (NM_001291931, | | |
| | | | | intron 6 of 10) | | |
| chr9 | 36748433 | 36748432 | hyper | Intergenic | 75164 | NR_039686 |
| chr9 | 38437314 | 38437313 | hyper | Intergenic | −12869 | NM_001007563 |
| chr9 | 93792388 | 93792387 | hyper | Intergenic | 45027 | NR_033912 |
| chr9 | 133769512 | 133769511 | hyper | intron | 2688 | NM198180 |
| | | | | (NM_198180, | | |
| | | | | intron 2 of 2) | | |
| chr9 | 138719304 | 138719303 | hyper | exon | 79702 | NM_015447 |
| | | | | (NM_015447, | | |
| | | | | exon 8 of 17) | | |
| chr1 | 202039153 | 202039152 | hypo | Intergenic | −52877 | NM_004767 |
| chr14 | 100039425 | 100039424 | hypo | intron | 31303 | NM_001144995 |
| | | | | (NM_001144995, | | |
| | | | | intron 1 of 5) | | |
| chr18 | 8660098 | 8660097 | hypo | Intergenic | 47522 | NR_120598 |
| chr18 | 14917016 | 14917015 | hypo | Intergenic | −29251 | NR_104164 |
| chr18 | 25442246 | 25442245 | hypo | Intergenic | 174304 | NM_001308176 |
| chr2 | 89530057 | 89530056 | hypo | Intergenic | 418172 | NR_039635 |
| chr20 | 20421462 | 20421461 | hypo | intron | 72696 | NM_002196 |
| | | | | (NM_020343, | | |
| | | | | intron 37 of 39) | | |
| chr20 | 21563921 | 21563920 | hypo | intron | 13258 | NR_109880 |
| | | | | (NR_109880, | | |
| | | | | intron 1 of 3) | | |
| chr21 | 46885392 | 46885391 | hypo | intron | 9967 | NM_130444 |
| | | | | (NM_130444, | | |
| | | | | intron 1 of 41) | | |
| chr3 | 193490472 | 193490471 | hypo | Intergenic | −145350 | NR_046634 |
| chr5 | 36521979 | 36521978 | hypo | Intergenic | −84479 | NM_001289940 |
| chr5 | 39390555 | 39390554 | hypo | exon | −25899 | NM_001737 |
| | | | | (NM_001343, | | |
| | | | | exon 5 of 15) | | |
| chr5 | 168573593 | 16857359 | hypo | intron | 117106 | NR_030311 |
| | | | | (NM_001271946, | | |
| | | | | intron 4 of 35) | | |
| chr7 | 2006579 | 2006578 | hypo | intron | −26392 | NM_001304525 |
| | | | | (NM_001304523, | | |
| | | | | intron 14 of 17) | | |
| chr7 | 129912398 | 129912397 | hypo | intron | 5694 | NM_001869 |
| | | | | (NM_001869, | | |
| | | | | intron 4 of 10) | | |
| chr8 | 61990390 | 61990389 | hypo | Intergenic | −110082 | NR_034003 |
| chr8 | 69087227 | 69087226 | hypo | intron | −155731 | NM_052958 |
| | | | | (NM_024870, | | |
| | | | | intron 35 of 39) | | |
| chr8 | 133070961 | 133070960 | hypo | intron | 667 | NM_001080399 |
| | | | | (NM_001080399, | | |
| | | | | intron 1 of 13) | | |
| chr8 | 141458926 | 141458925 | hypo | intron | 8936 | NM_001160372 |
| | | | | (NM_031466, | | |
| | | | | intron 2 of 22) | | |
| chr9 | 134913161 | 134913163 | hypo | intron | 42111 | NM_001253881 |
| | | | | (NM_001253881, | | |
| | | | | intron 2 of 6) | | |

| | | | | Gene |
|---|---|---|---|---|
| chr | Gene Name | Gene Alias | Gene Description | Type |
| chr1 | MIR3123 | mir-3123 | microRNA 3123 | ncRNA |
| chr18 | ACAA2 | DSAEC | acetyl-CoA | protein- |
| | | | acyltransferase 2 | coding |
| chr21 | KRTAP19-8 | — | keratin associated | protein- |
| | | | protein 19-8 | coding |
| chr5 | LPCAT1 | AYTL2\| | lysophosphatidylcholine | protein- |
| | | PFAAP3\| | acyltransferase 1 | coding |
| | | lpcat | | |
| chr5 | CARTPT | CART | CART | protein- |
| | | | prepropeptide | coding |
| chr5 | ARHGAP26 | GRAF\|GRAF1\| | Rho GTPase | protein- |
| | | OPHN1L\|OPHN1L1 | activating protein | coding |
| | | | 26 | |
| chr7 | RPA3 | REPA3 | replication protein | protein- |
| | | | A3 | coding |
| chr7 | MIR129-1 | MIR-129b\|MIRN129- | microRNA 129-1 | ncRNA |
| | | 1\|mir-129-1 | | |
| chr8 | FAM150A | UNQ9433 | family with | protein- |
| | | | sequence | coding |
| | | | similarity 150, | |
| | | | member A | |
| chr8 | KCNB2 | KV2.2 | potassium | protein- |
| | | | channel, voltage | coding |
| | | | gated Shab related | |
| | | | subfamily B, | |
| | | | member 2 | |
| chr8 | TERF1 | PIN2\|TRBF1\| | telomeric repeat | protein- |
| | | TRF\|TRF1\|hTRF1- | binding factor | coding |
| | | AS\|t-TRF1 | (NIMA- | |
| | | | interacting) 1 | |
| chr8 | KCNK9 | K2p9.1\|KT3.2\| | potassium | protein- |
| | | TASK-3\|TASK3 | channel, two pore | coding |
| | | | domain subfamily | |
| | | | K, member 9 | |
| chr8 | PTK2 | FADK\|FAK\| | protein tyrosine | protein- |
| | | FAK1\|FRNK\| | kinase 2 | coding |
| | | PPP1R71\|p125FAK\| | | |
| | | pp125FAK | | |
| chr8 | GPR20 | — | G protein-coupled | protein- |
| | | | receptor 20 | coding |
| chr8 | TSNARE1 | — | t-SNARE domain | protein- |
| | | | containing 1 | coding |
| chr9 | MIR4475 | — | microRNA 4475 | ncRNA |
| chr9 | IGFBPL1 | IGFBP-RP4\| | insulin like | protein- |
| | | IGFBPRP4\| | growth factor | coding |
| | | bA113O24.1 | binding protein- | |
| | | | like 1 | |
| chr9 | LOC100129316 | — | uncharacterized | ncRNA |
| | | | LOC100129316 | |
| chr9 | QRFP | 26RFa\|P518 | pyroglutamylated | protein- |
| | | | RFamide peptide | coding |
| chr9 | CAMSAP1 | PRO2405\| | calmodulin | protein- |
| | | bA100C15.1 | regulated | coding |
| | | | spectrin- | |
| | | | associated protein | |
| | | | 1 | |
| chr1 | GPR37L1 | ET(B)R-LP-2\| | G protein-coupled | protein- |
| | | ETBR-LP-2 | receptor 37 like 1 | coding |
| chr14 | CCDC85C | — | coiled-coil | protein- |
| | | | domain | coding |
| | | | containing 85C | |
| chr18 | GACAT2 | MTCL1-AS1\| | gastric cancer | ncRNA |
| | | MTCL1ASI | associated | |
| | | | transcript 2 (non- | |
| | | | protein coding) | |
| chr18 | LINC01443 | — | long intergenic | ncRNA |
| | | | non-protein | |
| | | | coding RNA 1443 | |
| chr18 | CDH2 | CD325\|CDHN\| | cadherin 2, type 1, | protein- |
| | | CDw325\|NCAD | N-cadherin | coding |
| | | | (neuronal) | |
| chr2 | MIR4436A | mir-4436a | microRNA 4436a | ncRNA |
| chr20 | INSM1 | IA-1\|IA1 | insulinoma- | protein- |
| | | | associated 1 | coding |
| chr20 | LOC101929625 | — | uncharacterized | ncRNA |
| | | | LOC101929625 | |
| chr21 | COL18A1 | KNO\|KNO1\|KS | collagen, type | protein- |
| | | | XVIII, alpha 1 | coding |
| chr3 | OPA1-AS1 | — | OPA1 antisense | ncRNA |
| | | | RNA 1 | |
| chr5 | SLC1A3 | EA6\|EAAT1\| | solute carrier | protein- |
| | | GLAST\|GLAST1 | family 1 (glial | coding |
| | | | high affinity | |
| | | | glutamate | |
| | | | transporter), | |
| | | | member 3 | |
| chr5 | C9 | ARMD15\|C9D | complement | protein- |
| | | | component 9 | coding |
| chr5 | MIR585 | MIRN585\|hsa-mir- | microRNA 585 | ncRNA |
| | | 585\|mir-585 | | |
| chr7 | MAD1L1 | MAD1\|PIG9\| | MAD 1 mitotic | protein- |
| | | IP53I9\|TXBP181 | arrest deficient- | coding |
| | | | like 1 (yeast) | |
| chr7 | CPA2 | — | carboxypeptidase | protein- |
| | | | A2 (pancreatic) | coding |
| chr8 | LOC100130298 | — | hCG1816373-like | ncRNA |
| chr8 | C8orf34 | VEST-1\|VEST1 | chromosome 8 | protein- |
| | | | open reading | coding |
| | | | frame 34 | |
| chr8 | OC90 | PLA2L | otoconin 90 | protein- |
| | | | | coding |
| chr8 | TRAPPC9 | IBP\|IKBKBBP\| | trafficking protein | protein- |
| | | MRT13\|NIBP\| | particle complex 9 | coding |
| | | T1\|TRS120 | | |
| chr9 | MED27 | CRAP34\|CRSP34\| | mediator complex | protein- |
| | | CRSP8\|TRAP37 | subunit 27 | coding |

i. Concordance of Differential Methylation in Matched Plasma and Tissue Samples

Differentially methylated regions (DMRs) were nominated using Rocker-meth. Starting from published enhanced reduced representation bisulfate sequencing of solid tumor biopsy samples and healthy prostate tissues (eRRBS-Sequencing (Beltran et al. (2016) Nat Med 22:298-305; Lin et al. (2013) Neoplasia 15:373-383)), two orthogonal differential methylation analysis were performed using default parameters values with the exception of na_threshold set to 0.3. First, DMRs between primary prostate cancer tissues (test, n=7) and normal prostatic tissue (control, n=7), (PCa|NT) were identified. Second, CRPC-NE (test, n=10) was compared to CRPC-Adeno (control, n=18), obtaining a set of DMRs which reflects methylation changes upon transdifferentiation (CRPC-NE|CRPC-Adeno). FDR of 0.01, an absolute average Beta difference of 20 between test and control and the presence of at least 6 detected CpG sites inside the region were applied as filters. Shared DMRs were isolated using bedtools -multiiterclust (Quinlan (2014) Curr Protoc Bioinformatics 47:11.12.1-34) and discarded. To capture single sample alterations of DNA methylation, z-scores for each DMR in each pair of tissue-plasma matched samples were computed by Rocker-meth using normal prostatic tissue as reference. To control for potential sources of noise due to non-tumoral cfDNA, the methylome of peripheral blood mono-nuclear cells (PBMC) of all 11 patients was also profiled using WGBS sequencing. For both DMR sets the z-scores detected across PBMC samples were evaluated and the DMRs that consistently deviated (abs(z-score)>5) from the reference in more than 80% (at least 9 out of 11) of PBMC samples were excluded. A total of 7,606 and 3,843 DMRs were obtained for PCa|NT and CRPC-NE|CRPC-Adeno, respectively (Table 4). To maximize the compatibility of different assays, only CpG sites detected in at least more than half of plasma samples (ctDNA and PBMC) were retained in single sample z-score computation, excluding sites that were detected only in eRRBS tissue samples. Furthermore, all DMRs containing less than 6 detected CpG sites in WGBS samples or with an undetectable signal in one or more WGBS plasma samples were excluded from pairwise tissue-plasma analysis and DMR score computation to ensure consistency between samples.

j. Integration of Plasma Methylomes from an Independent CRPC Cohort

To evaluate the robustness of the findings, a published cohort of cfDNA methylomes of patients was analyzed with CRPC treated with abiraterone and prednisone (Gordeviĉius et al. (2018) Clin Cancer Res 24:3317-3324) generated with the HM450 array, including only end of treatment (EoT) time-point data to maximize the similarity in disease state and clinical history. Using the previously defined set of CRPC-NE|CRPC-Adeno DMRs, single sample z-scores were computed for EoT time-points (using normal tissue samples from TCGA-PRAD as reference (Network (2015) Cell 163:1011-1025)). In order to obtain a comparable set of samples, only samples with TC (Benelli, Romagnoli, and Demichelis (2018) Bioinformatics 34:1642-1649) above or equal to 10% were retained in the analysis using a threshold similar to prior WES ctDNA studies (Adalsteinsson et al. (2017) Nat Commun 8:1324).

k. Differentially Methylated Regions

Given methylation values for each CpG site in a control and a test set of samples, the following procedure was applied: i) for each detected site, the area under the receiver operator characteristics was computed (AUROC), evaluating the segregation between groups based solely on target CpG signal. ii) Sequential AUROC values were divided into segments with a common methylation status (hyper-methylated, neutral or hypo-methylated) following a three-state heterogeneous shifting level model. iii) Significance of differential methylation between control and test group was assessed for each DMR with WMW-test on average Beta values, and p-values are corrected for multiple hypothesis testing with standard BH procedure.

l. Neuroendocrine Prostate Cancer (NEPC) Score

The NEPC score includes genomic and methylation-based features. Genomic features include RB1 deletion or mutation, TP53 deletion or mutation, AR focal gain or mutation, and CYLD deletion. TP53, RB1 and CYLD genomic based features for the NEPC feature score are set to one for a specific sample if the aberration is present; AR genomic feature is instead set to one for a specific sample in case of absence of aberrations. Methylation based signal includes two components based on top 20 hypo- and 20 hyper-methylated sites based on previous tissue analysis (Table 6). Briefly, differentially methylated sites from Beltran et al. (Table 7, from Beltran et al. (2016) Nat Med 22:298-305) were ranked by FDR and by beta difference (CRPC-NE vs CRPC-Adeno) and retained only those sites found in WGBS data. To avoid redundant signal, the top-ranking one was retained if sites were within 10 Kp from each other. Next, to have comparable methylation levels from tissue and plasma samples, a purity correction of M-values was applied using PAMES. Then, for each top hyper- and hypo-methylated site, the threshold that best discriminated between CRPC-Adeno and CRPC-NE tissue samples was identified by Receiver operating characteristic (ROC) curve analysis. Last, plasma methylation data were dichotomized as follows: for each plasma sample, methylation feature score is equal to 1 if the majority of sites show methylation level above (hyper) or below (hypo) the site specific tissue-based threshold. The NEPC feature score was computed for a specific sample by counting the number of features set to one and normalizing by the number of available features for that sample. Features with no call were set to NA and were not considered in the score computation.

m. Statistical Analysis

Association of plasma genomics and binary clinical variables was performed using two-tailed Wilcoxon-Mann-Whitney test with significance level set at 5%. Association of plasma genomics and continuous clinical variables was performed using Pearson correlation statistics with significance level set at 5%. Statistical comparison of genomic variables across sample's classes was performed using two-tailed Wilcoxon-Mann-Whitney test with significance level set at 5%. Univariate overall survival and progression-free survival analyses were performed using the Kaplan-Meier estimator (log-rank test). Multivariate overall and progression-free survival analyses were performed using a proportional hazard model with stepwise model selection by Akaike information criterion using forward and backward directions.

n. Study Approval

This study was approved by the institutional review board (WCM IRB #1305013903, #1210013164) and written informed consent was obtained from participants prior to inclusion in the study.

All BAM files generated for this study and associated sample information are described in Tables 2 and 8. and accessible through dbGaP (phs001752.v1.p1): available on the World Wide Web at ncbi.nlm.nih.gov/projects/gap/cgibin/study.cgi?study_id=phs001752.v1.p1.

### Example 2: Identification of Genomic and Epigenomic Features of CRPC-NE

It was hypothesized that while tumors do acquire specific CRPC-NE changes (predominantly epigenetic) during the process of lineage plasticity, early resistant or aggressive prostate cancer clones (such as those already harboring loss of RB1 and TP53) may also gain clonal dominance during CRPC-NE transformation and may be early selected facilitators of transdifferentiation. While metastatic lesions in prostate cancer may harbor evidence of subclonal heterogeneity across sites potentially due to metastasis-to-metastasis seeding as observed in prior studies (Gundem et al. (2015) Nature 520:353-357), it was predicted that tumors would become less heterogeneous as patients progress towards CRPC-NE in later stages of the disease (as seen in rapid autopsy studies (Kumar et al. (2016) Nat Med 22:369-378)). To test this hypothesis, tumor DNA detectable in the circulation of patients was studied with CRPC-NE and CRPC-Adeno and compared their genomic and epigenomic profiles with patient-matched tumor biopsies.

In addition to using circulating tumor DNA (ctDNA) as a tool to study heterogeneity, the potential utility of this approach as a noninvasive tool for the detection of CRPC-NE was explored by querying specific genomic and epigenomic changes seen in CRPC-NE tumors (Abida et al. (2019) Proc Natl Acad Sci USA 116:11428-11436; Aggarwal et al. (2018) J Clin Oncol JCO201777 6880; Beltran et al. (2016) Nat Med 22:298-305) in the circulation of biopsy-confirmed patients. A circulating biomarker for CRPC-NE has potential clinical implications that can lead to future avoidance of invasive biopsy for patients (the current standard for CRPC-NE diagnosis). Furthermore, since there is a spectrum of histologies even within CRPC-NE and as patients progress from adenocarcinoma to CRPC-NE, with mixed adenocarcinoma-neuroendocrine morphologies and hybrid tumors with overlapping features sometimes observed (Beltran et al. (2019) Clin Cancer Res 25:6916-6924; Epstein et al. (2014) Am J Surg Pathol 38:756-767), identifying molecular features could provide additional information above or complementary to a tumor biopsy. Noninvasive detection of molecular features of CRPC-NE can therefore lead to a more refined subclassification and detect features in patients at high risk for transformation even without CRPC-NE histology, paving the way for early intervention treatment strategies.

Plasma blood samples and matched metastatic tumor biopsies were obtained from sixty-two men with metastatic prostate cancer (10 hormone naive metastatic prostate adenocarcinoma (mPCA), 35 castration resistant prostate adenocarcinoma (CRPC-Adeno) and 17 CRPC-NE) classified histologically using published criteria (Epstein et al. (2014) Am J Surg Pathol 38:756-767). Clinical features are summarized in Table 9 and FIG. 1A. Twenty-four of these individuals had multiple tissue or plasma time-points, for a total of 69 plasma samples and 98 metastatic tissues (Tables 2, and 9-11). Metastatic sites of patients at the time of specimen collection included bone (84%), lymph node (52%), liver (32%), lung (16%), brain (6%). Overall patients in this study had high metastatic burden, including 50% with visceral metastases and >45% of patients with >6 bone metastases. The median number of prior therapies for CRPC was two (range 0-9); 42.8% patients received prior abiraterone or enzalutamide, and 65.7% received prior cytotoxic chemotherapy. The median time between prior therapy progression and blood draw was 3.2 months (range 1.1-9.1). Median progression free survival on next line of systemic therapy (after blood/tissue collection) for CRPC patients was 4.1 months (4.3 months CRPC-Adeno, 4.0 months CRPC-NE), and median overall survival was 11.0 months (12.0 months CRPC-Adeno, 6.0 months CRPC-NE).

TABLE 9
Patient demographics

| | Overall | | CRPC-Adeno | | CRPC-NE | |
|---|---|---|---|---|---|---|
| | (n = 62) | | (n = 35) | | (n = 17) | |
| Age at diagnosis of PCA, years | 65 | (50-90) | 62 | (50-90) | 68 | (54-90) |
| Median (range) | | | | | | |
| Prostatectomy, n (%) | 21 | (33.9) | 15 | (42.9) | 5 | (29.4) |
| Radical radiotherapy, n (%) | 24 | (38.7) | 12 | (34.3) | 7 | (41.2) |
| Gleason sum at diagnosis, n (%) | | | | | | |
| ≤7 | 21 | (38.9) | 11 | (32.4) | 6 | (50) |
| ≥8 | 33 | (61.1) | 23 | (67.6) | 6 | (50) |
| Unknown/missing | 8 | | 1 | | 5 | |
| Classification at plasma | | | | | | |
| collection, n (%) | | | | | | |
| CRPC-Adeno | 35 | (56.5) | 35 | (100) | 0 | (0) |
| CRPC-NE | 17 | (27.4) | 0 | (0) | 17 | (100) |
| HN met PCA | 10 | (16.1) | 0 | (0) | 0 | (0) |
| Sites of metastases, n (%) | | | | | | |
| Bone | 52 | (83.9) | 30 | (85.7) | 12 | (70.6) |
| Lymph node | 35 | (56.5) | 21 | (60) | 9 | (53) |
| Visceral | 26 | (41.9) | 14 | (40) | 9 | (53) |
| Liver | 22 | (35.5) | 11 | (31.4) | 9 | (53) |
| Lung | 11 | (17.7) | 6 | (17.1) | 4 | (23.6) |
| Brain | 4 | (6.5) | 3 | (8.6) | 1 | (5.9) |
| Other | 4 | (6.5) | 2 | (5.7) | 2 | (11.8) |
| Time from PCA diagnosis to | | | | | | |
| castration-resistance, weeks | | | | | | |
| Median (range) | 85 | (1-915) | 102 | (6-915) | 79 | (1-279) |
| Time from PCA diagnosis | | | | | | |
| to metastasis, weeks | | | | | | |
| Median (range) | 9.5 | (0-987) | 18 | (0-987) | 2 | (0-423) |
| Number of systemic treatments | | | | | | |
| before plasma collection, n (%) | | | | | | |
| ≤2 | 37 | (59.7) | 16 | (45.7) | 12 | (70.6) |
| ≥3 | 25 | (40.3) | 19 | (54.3) | 5 | (29.4) |
| Type of prior systemic treatments | | | | | | |
| before plasma collection, n (%) | | | | | | |
| Anti-androgens | | | | | | |
| (bicalutamide, nilutamide, flutamide) | 21 | (33.9) | 17 | (48.6) | 2 | (11.8) |
| LHRH agonists (leuprolide, goserelin) | 29 | (46.8) | 23 | (65.7) | 5 | (29.4) |
| LHRH antagonists (degarelix) | 20 | (32.2) | 12 | (34.3) | 6 | (35.3) |
| Sipuleucel T | 12 | (19.4) | 12 | (34.3) | 0 | (0) |
| Docetaxel | 16 | (25.8) | 12 | (34.3) | 3 | (17.6) |
| Abiraterone | 16 | (25.8) | 14 | (40) | 1 | (5.9) |
| Enzalutamide | 4 | (6.5) | 4 | (11.4) | 0 | (0) |
| Cabazitaxel | 4 | (6.5) | 4 | (11.4) | 0 | (0) |
| Radium-223 | 3 | (4.8) | 3 | (8.6) | 0 | (0) |
| 177-Lutetium PSMA therapy | 6 | (9.7) | 6 | (17.1) | 0 | (0) |
| Platinum-based therapy | 10 | (16.1) | 2 | (5.7) | 8 | (47.1) |
| Other (ketoconazole, cabozantinib) | 1 | (1-6) | 1 | (2.9) | 0 | (0) |
| Treatments at time of plasma | | | | | | |
| collection, n (%) | | | | | | |
| Anti-androgens (bicalutamide) | 4 | (6.5) | 0 | (0) | 1 | (5.9) |
| LHRH agonists (leuprolide) | 5 | (8.1) | 0 | (0) | 1 | (5.9) |
| LHRH antagonists (degarelix) | 4 | (6.5) | 0 | (0) | 0 | (0) |
| Sipuleucel T | 0 | (0) | 0 | (0) | 0 | (0) |
| Docetaxel | 7 | (11.3) | 6 | (17.1) | 0 | (0) |
| Abiraterone | 12 | (19.4) | 9 | (25.7) | 3 | (17.6) |
| Enzalutamide | 6 | (9.7) | 5 | (14.3) | 0 | (0) |
| Cabazitaxel | 4 | (6.5) | 4 | (11.4) | 0 | (0) |
| 177-Lutetium PSMA therapy | 3 | (4.8) | 3 | (8.6) | 0 | (0) |
| Platinum-based therapy | 4 | (6.5) | 1 | (2.9) | 3 | (17.6) |
| Mitoxantrone | 1 | (1-6) | 1 | (2.9) | 0 | (0) |
| Alisertib | 14 | (22.6) | 6 | (17.1) | 8 | (47.1) |
| PSA before plasma collection, ng/dL | 12.1 | (0.01-7527) | 17.5 | (0.5-7527) | 0.58 | (0.01-110.9) |
| Median (range) | | | | | | |
| ALP before plasma collection, U/L | 96 | (35-1009) | 92 | (42-821) | 109 | (38-444) |
| Median (range) | | | | | | |
| Hemoglobin before plasma | 11.8 | (7.1-14.7) | 11.9 | (7.1-14.3) | 11 | (8.1-13.1) |
| collection, g/dL | | | | | | |
| Median (range) | | | | | | |
| Albumin before plasma | 3.8 | (2.2-4.7) | 3.9 | (2.2-4.7) | 3.2 | (2.8-4.1) |
| collection, g/dL | | | | | | |
| Median (range) | | | | | | |
| LDH before plasma collection, n (%) | | | | | | |
| <192 U/l | 36 | (64.3) | 20 | (62.5) | 9 | (64.3) |
| ≥192# U/l | 20 | (35.7) | 12 | (37.5) | 5 | (35.7) |
| Unknown/missing | 6 | | 3 | | 3 | |
| Serum Neuroendocrine Markers | | | | | | |
| CGA before plasma collection, n (%) | | | | | | |
| <95 ng/mL | 11 | (25) | 7 | (31.8) | 1 | (6.7) |
| ≥95# ng/mL | 33 | (75) | 15 | (68.2) | 14 | (93.3) |
| Unknown/missing | 18 | | 13 | | 2 | |
| NSE before plasma collection, n (%) | | | | | | |
| <8.9 mg/L | 22 | (57.9) | 13 | (65) | 7 | (46.7) |
| ≥8.9# mg/L | 16 | (42.1) | 7 | (35) | 8 | (53.3) |

| | | | |
|---|---|---|---|
| Unknown/missing | 24 | 15 | 2 |

* Upper normal value

Abbreviations: ALP, alkaline phosphatase; CGA, chromogranin A; CRPC-Adeno, castration-resistant prostate adenocarcinoma; CRPC-NE, castration resistant prostate adenocarcinoma with neuroendocrine features; HN met PCA, hormone-naïve metastatic prostate cancer; LDH, lactate dehydrogenase; LHRH, luteinizing hormone-releasing hormone; NSE, neuron-specific enolase; PCA, prostate carcinoma; PSA, prostate specific antigen; PSMA, prostate-specific membrane antigen.

TABLE 10
Plasma Samples Clinical Variables

| | | CRPC at | | | | |
|---|---|---|---|---|---|---|
| | | time of | | | therapy at | |
| | | plasma | | | plasma | |
| | Age at | collection | Class at | Plasma | collection | |
| | Plasma | no = 0, | plasma | collection | time | |
| Sample | Collection | yes = 1 | collection | date | (baseline) | |
| WCM0 | 56 | 0 | CRPC-NE | Jan. 3, 2013 | carboplatin etoposide | |
| WCM1 | 72 | 0 | HNPC | Feb. 6, 2013 | degarelix | |
| WCM2 | 73 | 1 | CRPC-Adeno | Feb. 21, 2013 | docetaxel | |
| WCM4 | 59 | 0 | HNPC | Mar. 28, 2013 | leuprolide, bicalutamide | |
| WCM6 | 76 | 1 | CRPC-Adeno | Mar. 28, 2013 | alisertib | |
| WCM7 | 63 | 1 | CRPC-Adeno | Apr. 9, 2013 | etoposide cisplatino | |
| WCM9 | 64 | 1 | CRPC-Adeno | Apr. 29, 2013 | abiraterone | |
| WCM10 | 86 | 1 | CRPC-NE | Apr. 24, 2013 | alisertib | |
| WCM12 | 67 | 1 | CRPC-NE | Jun. 4, 2013 | no systemic therapy | |
| | | | | | (radiotherapy cyberknife | |
| | | | | | 5/13) | |
| WCM14-TP1 | 69 | 1 | CRPC-Adeno | Apr. 26, 2017 | mitoxantrone | |
| WCM14-TP2 | 69 | 1 | CRPC-Adeno | May 10, 2017 | mitoxantrone | |
| WCM14-TP3 | 69 | 1 | CRPC-Adeno | Jun. 7, 2017 | mitoxantrone | |
| WCM14-TP4 | 70 | 1 | CRPC-Adeno | Jun. 28, 2017 | mitoxantrone | |
| WCM15 | 74 | 1 | CRPC-NE | Jun. 11, 2013 | alisertib | |
| WCM63 | 58 | 1 | CRPC-Adeno | Sep. 20, 2013 | docetaxel (+MoAb J591) | |
| WCM73 | 87 | 0 | HNPC | Aug. 28, 2013 | degarelix + leuprolide | |
| WCM78 | 79 | 1 | CRPC-Adeno | Nov. 12, 2013 | alisertib | |
| WCM80 | 65 | 0 | HNPC | Sep. 26, 2013 | leuprolide bicalutamide | |
| WCM89 | 54 | 1 | CRPC-Adeno | Sep. 24, 2013 | cabazitaxel | |
| WCM90 | 62 | 1 | CRPC-Adeno | Oct. 8, 2013 | no systemic therapy | |
| | | | | | (bilateral orchiectomy | |
| | | | | | 8/13) | |
| WCM91 | 57 | 1 | CRPC-Adeno | Sep. 3, 2014 | docetaxel | |
| WCM119 | 66 | 1 | CRPC-Adeno | Nov. 21, 2013 | abiraterone | |
| WCM121 | 69 | 1 | CRPC-Adeno | Oct. 16, 2013 | enzautamide | |
| WCM124 | 96 | 1 | CRPC-Adeno | Oct. 18, 2013 | abiraterone | |
| WCM131 | 59 | 0 | HNPC | Dec. 3, 2013 | degarelix | |
| WCM139 | 77 | 1 | CRPC-Adeno | Dec. 4, 2013 | enzalutamide | |
| WCM142 | 78 | 1 | HNPC | Dec. 19, 2013 | enzalutamide | |
| WCM154 | 79 | 1 | CRPC-NE | Jan. 10, 2014 | alisertib | |
| WCM155 | 87 | 1 | CRPC-NE | Jan. 8, 2014 | alisertib | |
| WCM156 | 62 | 1 | CRPC-Adeno | Dec. 19, 2013 | alisertib | |
| WCM158 | 65 | 1 | CRPC-Adeno | Jan. 28, 2014 | Sipuleucel T | |
| WCM160 | 73 | 0 | CRPC-NE | Feb. 12, 2014 | abiraterone | |
| WCM161 | 66 | 1 | CRPC-Adeno | Feb. 13, 2014 | alisertib | |
| WCM162 | 67 | 1 | CRPC-Adeno | Feb. 18, 2014 | docetaxel | |
| WCM163 | 68 | 1 | CRPC-NE | Mar. 5, 2014 | abiraterone | |
| WCM164 | 81 | 1 | CRPC-Adeno | Feb. 18, 2014 | Lu 177 DOTA-JR11 | |
| WCM169 | 66 | 0 | CRPC-Adeno | Apr. 15, 2014 | docetaxel gemcitabine | |
| WCM170 | 71 | 1 | CRPC-Adeno | Mar. 20, 2014 | abiraterone | |
| WCM172 | 69 | 1 | CRPC-Adeno | Mar. 18, 2014 | abiraterone | |
| WCM176 | 74 | 0 | HNPC | Dec. 15, 2014 | leuprolide, bicalutamide | |
| WCM183 | 71 | 1 | CRPC-Adeno | Apr. 15, 2014 | enzalutamide | |
| WCM185 | 65 | 1 | CRPC-Adeno | Apr. 30, 2014 | cabazitaxel | |
| WCM185-TP1 | 69 | 1 | CRPC-Adeno | May 17, 2017 | mitoxantrone | |
| WCM185-TP2 | 69 | 1 | CRPC-Adeno | May 24, 2017 | mitoxantrone | |
| WCM185-TP3 | 69 | 1 | CRPC-Adeno | May 31, 2017 | mitoxantrone | |
| WCM185-TP4 | 69 | 1 | CRPC-Adeno | Jun. 13, 2017 | mitoxantrone | |
| WCM192 | 67 | 0 | CRPC-NE | Feb. 5, 2015 | alisertib | |
| WCM195 | 86 | 1 | CRPC-Adeno | Apr. 29, 2014 | docetaxel | |
| WCM198 | 66 | 0 | CRPC-NE | Nov. 26, 2013 | alisertib | |
| WCM199 | 67 | 1 | CRPC-Adeno | Dec. 23, 2013 | alisertib | |
| WCM201 | 58 | 0 | HNPC | May 12, 2014 | NA | |
| WCM207 | 69 | 0 | CRPC-NE | May 7, 2014 | alisertib | |
| WCM212 | 59 | 1 | CRPC-NE | May 12, 2014 | NA | |
| WCM213 | 71 | 1 | CRPC-Adeno | May 20, 2014 | cabazitaxel | |
| WCM216 | 69 | 1 | CRPC-Adeno | Jul. 3, 2014 | abiraterone + enzalutamide | |
| WCM218 | 64 | 1 | CRPC-Adeno | Sep. 16, 2014 | 177Lu-J591 | |
| WCM219 | 67 | 1 | CRPC-Adeno | Jul. 8, 2014 | abiraterone enzalutamide | |
| WCM223 | 62 | 1 | CRPC-Adeno | Nov. 25, 2014 | enzalutamide | |
| WCM243 | 78 | 1 | CRPC-NE | Aug. 20, 2014 | abiraterone | |
| WCM247 | 86 | 0 | HNPC | Sep. 11, 2014 | degarelix | |
| WCM252 | 68 | 1 | CRPC-NE | Sep. 3, 2014 | alisertib | |
| WCM253 | 74 | 1 | CRPC-NE | Oct. 22, 2014 | carboplatin paclitaxel | |
| WCM261 | 75 | 1 | CRPC-Adeno | Oct. 28, 2014 | abiraterone + prednisone + | |
| | | | | | GDC0068/placebo | |
| WCM341 | 72 | 1 | CRPC-Adeno | Nov. 17, 2014 | alisertib | |
| WCM365 | 80 | 1 | CRPC-Adeno | Mar. 18, 2015 | NA | |
| WCM366 | 64 | 0 | HNPC | Mar. 26, 2015 | docetaxel | |
| WCM369 | 52 | 1 | CRPC-Adeno | Jun. 2, 2015 | abiraterone (+cabazitaxel) | |
| WCM677 | 93 | 0 | CRPC-NE | Dec. 22, 2015 | bicalutamide leuprolide | |
| | | | | | (Microwave ablation | |
| | | | | | Nov. 13, 2015) | |
| WCM1388 | 81 | 1 | CRPC-NE | Aug. 1, 2017 | Carboplatin/ | |
| | | | | | Paclitaxel | |
| | | | | | number of | |
| | | | | | previous | |
| | | | | | tp lines | |
| | | | | censor | (before | Type of |
| | start date | stop date | date of | (no = 0, | plasma | previous |
| Sample | of therapy | of therapy | progression | yes = 1) | collection) | tp lines |
| WCM0 | Dec. 7, 2012 | Jan. 9, 2013 | Jan. 9, 2013 | 1 | 0 | NA |
| WCM1 | Feb. 8, 2013 | Aug. 5, 2014 | Aug. 5, 2014 | 1 | 1 | docetaxel |
| WCM2 | Feb. 21, 2013 | Jul. 14, 2013 | Jul. 14, 2013 | 1 | 2 | degarelix, abiraterone |
| WCM4 | Apr. 24, 2013 | Aug. 20, 2013 | Aug. 20, 2013 | 1 | 0 | NA |
| WCM6 | Mar. 28, 2013 | Jul. 23, 2013 | Jul. 23, 2013 | 1 | 5 | leuprolide, bicalutamide, |
| | | | | | | ketoconazolo, docetaxel, |
| | | | | | | abiraterone |
| WCM7 | Apr. 1, 2013 | May 28, 2013 | Jun. 18, 2013 | 1 | 6 | degarelix, bicalutamide, docetaxel, abiraterone, |
| | | | | | | enzalutamide, cabazitaxel |
| WCM9 | Aug. 21, 2013 | Feb. 4, 2014 | Feb. 4, 2014 | 1 | 2 | degarelix, bicalutamide |
| WCM10 | May 3, 2013 | Feb. 2, 2017 | Feb. 2, 2017 | 1 | 1 | leuprolide |
| WCM12 | NA | NA | NA | NA | 0 | NA |
| WCM14-TP1 | May 10, 2017 | Jun. 28, 2017 | Jun. 28, 2017 | 1 | 9 | leuprolide, bicalutamide, bicalutamide, abiraterone, |
| | | | | | | radium223, docetaxel, enzlautamide, cabazitaxel, |
| | | | | | | docetaxel |
| WCM14-TP2 | May 10, 2017 | Jun. 28, 2017 | Jun. 28, 2017 | 1 | 9 | leuprolide, bicalutamide, bicalutamide, abiraterone, |
| | | | | | | radium223, docetaxel, enzlautamide, cabazitaxel, |
| | | | | | | docetaxel |
| WCM14-TP3 | May 10, 2017 | Jun. 28, 2017 | Jun. 28, 2017 | 1 | 9 | leuprolide, bicalutamide, bicalutamide, abiraterone, |
| | | | | | | radium223, docetaxel, enzlautamide, cabazitaxel, |
| | | | | | | docetaxel |
| WCM14-TP4 | May 10, 2017 | Jun. 28, 2017 | Jun. 28, 2017 | 1 | 9 | leuprolide, bicalutamide, bicalutamide, abiraterone, |
| | | | | | | radium223, docetaxel, enzlautamide, cabazitaxel, |
| | | | | | | docetaxel |
| WCM15 | Jun. 12, 2013 | Jan. 15, 2014 | Jan. 15, 2014 | 1 | 2 | ADT, cisplatin etoposide |
| WCM63 | Oct. 1, 2013 | Feb. 22, 2014 | Mar. 7, 2015 | 1 | 3 | degarelix, bicalutamide, sipuleucel T |
| WCM73 | Aug. 28, 2013 | Sep. 11, 2014 | Sep. 11, 2014 | 0 | 0 | NA |
| WCM78 | Nov. 4, 2013 | Mar. 3, 2014 | Mar. 3, 2014 | 1 | 2 | degarelix, abiraterone |
| WCM80 | Oct. 29, 2013 | Jun. 13, 2017 | Jun. 13, 2017 | 0 | 1 | degarelix |
| WCM89 | Oct. 2, 2013 | May 8, 2014 | May 8, 2014 | 1 | 4 | leuprolide, ketoconazolo, sipuleucel T, MoAb J591 |
| WCM90 | NA | NA | NA | NA | 4 | degarelix, bicalutamide, docetaxel, abiraterone |
| WCM91 | Sep. 17, 2014 | Dec. 1, 2014 | Dec. 1, 2014 | 1 | 2 | degarelix, abiraterone |
| WCM119 | Nov. 4, 2013 | Feb. 6, 2014 | Feb. 6, 2014 | 1 | 5 | leuprolide, bicalutamide, sipuleucel T, docetaxel, |
| | | | | | | enzalutamide |
| WCM121 | Aug. 24, 2013 | Nov. 4, 2013 | Nov. 4, 2013 | 1 | 5 | leuprolide, bicalutamide, nilutamide, sipuleucel T, |
| | | | | | | MoAb J591 |
| WCM124 | Sep. 22, 2013 | Apr. 3, 2014 | Apr. 3, 2014 | 1 | 4 | leuprolide, bicalutamide, sipuleucel T, MoAb J592 |
| WCM131 | Nov. 6, 2013 | Feb. 18, 2014 | Feb. 18, 2014 | 1 | 0 | NA |
| WCM139 | Nov. 15, 2013 | Dec. 24, 2013 | Dec. 24, 2013 | 1 | 3 | goserelin, bicalutamide, abiraterone |
| WCM142 | Dec. 29, 2013 | Mar. 1, 2014 | Mar. 1, 2014 | 1 | 3 | leuprolide, bicalutamide, abiraterone |
| WCM154 | Jan. 10, 2014 | Mar. 31, 2014 | Mar. 31, 2014 | 1 | 2 | degarelix, cisplatin etoposide |
| WCM155 | Jan. 16, 2014 | Mar. 17, 2015 | Mar. 17, 2015 | 1 | 1 | leuprolide |
| WCM156 | Nov. 22, 2013 | Jan. 17, 2014 | Jan. 17, 2014 | 1 | 4 | leuprolide, cisplatin etoposide, topotecan, |
| | | | | | | temozolomide etoposide |
| WCM158 | Apr. 19, 2014 | May 19, 2014 | Jul. 23, 2014 | 1 | 2 | leuprolide, bicalutamide |
| WCM160 | Nov. 11, 2014 | Feb. 24, 2015 | Feb. 24, 2015 | 1 | 3 | leuprolide, bicalutamide, carboplatin etoposide |
| WCM161 | May 21, 2014 | Jul. 22, 2014 | Jul. 22, 2014 | 1 | 5 | leuprolide, bicalutamide, MoAb J591, docetaxel, |
| | | | | | | abiraterone |
| WCM162 | Feb. 14, 2014 | Apr. 29, 2014 | Jul. 3, 2014 | 1 | 1 | leuprolide |
| WCM163 | Oct. 23, 2013 | Feb. 12, 2014 | Feb. 12, 2014 | 1 | 1 | degarelix |
| WCM164 | Mar. 27, 2014 | Apr. 7, 2014 | Apr. 7, 2014 | 1 | 4 | leuprolide, docetaxel, enzalutamide, radium223 |
| WCM169 | Dec. 11, 2014 | Feb. 12, 2015 | Jul. 3, 2015 | 1 | 2 | leuprolide, degarelix |
| WCM170 | Oct. 2, 2012 | Jul. 10, 2014 | Jul. 10, 2014 | 1 | 1 | degarelix |
| WCM172 | Dec. 26, 2012 | Mar. 26, 2015 | Mar. 26, 2015 | 1 | 3 | leuprolide, sipuleucel T, docetaxel |
| WCM176 | Jun. 16, 2014 | Dec. 18, 2017 | Dec. 18, 2017 | 0 | 2 | degarelix, bicalutamide |
| WCM183 | Apr. 15, 2014 | Feb. 18, 2015 | Feb. 18, 2015 | 1 | 8 | leuprolide, goserelin, MoAb J591, docetaxel, |
| | | | | | | cabazitaxel, abiraterone, cabozantinib, radium223 |
| WCM185 | Oct. 22, 2014 | Sep. 16, 2015 | Sep. 16, 2015 | 1 | 8 | leuprolide, bicalutamide, flutamide, MoAb J591, |
| | | | | | | sipuleucel T, abiraterone |
| WCM185-TP1 | May 24, 2017 | Jul. 19, 2017 | Jul. 19, 2017 | 1 | 9 | leuprolide, bicalutamide, flutamide, MoAb J591, |
| | | | | | | sipuleucel T, abiraterone, enzalutamide, docetaxel, |
| | | | | | | 177Lu PSMA |
| WCM185-TP2 | May 24, 2017 | Jul. 19, 2017 | Jul. 19, 2017 | 1 | 9 | leuprolide, bicalutamide, flutamide, MoAb J591, |
| | | | | | | sipuleucel T, abiraterone, enzalutamide, docetaxel, |
| | | | | | | 177Lu PSMA |
| WCM185-TP3 | May 24, 2017 | Jul. 19, 2017 | Jul. 19, 2017 | 1 | 9 | leuprolide, bicalutamide, flutamide, MoAb J591, |
| | | | | | | sipuleucel T, abiraterone, enzalutamide, docetaxel, |
| | | | | | | 177Lu PSMA |
| WCM185-TP4 | May 24, 2017 | Jul. 19, 2017 | Jul. 19, 2017 | 1 | 9 | leuprolide, bicalutamide, flutamide, MoAb J591, |
| | | | | | | sipuleucel T, abiraterone, enzalutamide, docetaxel, |
| | | | | | | 177Lu PSMA |
| WCM192 | Feb. 6, 2015 | Jun. 9, 2015 | Jun. 9, 2015 | 1 | 3 | Degarelix, cisplatin docetaxel, cisplatin etoposide |
| WCM195 | Mar. 11, 2014 | May 27, 2014 | May 27, 2014 | 1 | 2 | leuprolide, abiraterone |
| WCM198 | Nov. 26, 2013 | Feb. 28, 2014 | Feb. 28, 2014 | 1 | 2 | cisplatin etoposide, topotecan |
| WCM199 | Dec. 23, 2013 | Feb. 28, 2014 | Feb. 28, 2014 | 1 | 0 | NA |
| WCM201 | NA | NA | NA | NA | 0 | NA |
| WCM207 | May 8, 2014 | Jul. 3, 2014 | Jul. 3, 2014 | 1 | 2 | degarelix, carboplatin etoposide |
| WCM212 | NA | NA | NA | NA | 4 | leuprolide, abiraterone, docetaxel, carboplatin |
| | | | | | | etoposide |
| WCM213 | Jun. 17, 2014 | Dec. 1, 2014 | Dec. 1, 2014 | 1 | 4 | leuprolide, bicalutamide, docetaxel, abiraterone |
| WCM216 | Jun. 5, 2014 | Jan. 11, 2018 | Jan. 11, 2018 | 0 | 2 | leuprolide, sipuleucel T |
| WCM218 | Nov. 7, 2014 | Feb. 9, 2015 | Feb. 9, 2015 | 1 | 1 | degarelix |
| WCM219 | Jun. 5, 2014 | Aug. 13, 2014 | May 1, 2017 | 0 | 2 | degarelix, sipuleucel T |
| WCM223 | Apr. 11, 2014 | Jan. 5, 2017 | Jan. 5, 2017 | 1 | 3 | degarelix, bicalutamide, sipuleucel T |
| WCM243 | Aug. 23, 2014 | Feb. 18, 2015 | Feb. 18, 2015 | 1 | 1 | degarelix |
| WCM247 | Aug. 14, 2014 | Feb. 22, 2016 | Feb. 22, 2016 | 1 | 0 | NA |
| WCM252 | Sep. 3, 2014 | Nov. 5, 2014 | Nov. 5, 2014 | 1 | 3 | degarelix, cisplatin etoposide, topotecan |
| WCM253 | Sep. 25, 2014 | Jan. 15, 2015 | Feb. 3, 2015 | 1 | 3 | leuprolide, bicalutamide, docetaxel |
| WCM261 | Oct. 16, 2014 | Jan. 16, 2018 | Jan. 16, 2018 | 0 | 2 | leuprolide, docetaxel |
| WCM341 | Nov. 25, 2014 | Feb. 18, 2015 | Feb. 18, 2015 | 1 | 2 | leuprolide, bicalutamide |
| WCM365 | NA | NA | NA | NA | 9 | leuprolide, bicalutamide, abiraterone, sipuleucel, |
| | | | | | | docetaxel, cabazitaxel, enzalutamide, carboplatin, |
| | | | | | | 177Lu-J591 |
| WCM366 | Mar. 26, 2015 | Jul. 9, 2015 | Mar. 25, 2016 | 1 | 0 | NA |
| WCM369 | Apr. 14, 2015 | Nov. 29, 2016 | Nov. 29, 2016 | 1 | 2 | leuprolide, sipuleucel T |
| WCM677 | Sep. 24, 2013 | Apr. 12, 2016 | Apr. 12, 2016 | 1 | 1 | degarelix |
| WCM1388 | Aug. 11, 2017 | Dec. 10, 2017 | Dec. 10, 2017 | 1 | 0 | NA |
| | | | | | HEMO- | |
| | | Number | | | GLOBIN | SERUM |
| | Prior | of next | | | (range | ALBUMIN |
| | chemo. | line | Type of | | 13.3-17.7 | (range 3.5- |
| Sample | Y/N | therapy | Next Line Therapy | | g/dL) | 4.8 g/dL) |
| WCM0 | 0 | 0 | NA | | 13.1 | 3 |
| WCM1 | 1 | 1 | Docetaxel | | 12.5 | 3.1 |
| WCM2 | 0 | 3 | enzalutamide, carboplatin | | 11.8 | NA |
| | | | taxolo, cabozantinib | | | |
| WCM4 | 0 | 4 | docetaxel, abiraterone, | | 13.8 | 3.6 |
| | | | cabazitaxel, cisplatin | | | |
| | | | eoposide | | | |
| WCM6 | 1 | 2 | enzalutamide, | | 11.3 | 3.7 |
| | | | carboplatin taxolo | | | |
| WCM7 | 1 | 1 | Carboplatin taxolo | | 11.13 | 3.1 |
| WCM9 | 0 | 3 | docetaxel, enzalutamide, | | 13.3 | 4.2 |
| | | | cisplatin eoposide | | | |
| WCM10 | 0 | 1 | enzalutamide | | NA | 4.1 |
| WCM12 | 0 | 3 | docetaxel cisplatin, | | 11.3 | 3.9 |
| | | | carboplatin, | | | |
| | | | etoposide Carboplatin | | | |
| WCM14-TP1 | 1 | 0 | NA | | 9.8 | 2.2 |
| WCM14-TP2 | 1 | 0 | NA | | 9.2 | 2.5 |
| WCM14-TP3 | 1 | 0 | NA | | 6.9 | 2.4 |
| WCM14-TP4 | 1 | 0 | NA | | 8.7 | 2.6 |
| WCM15 | 1 | 1 | leuprolide | | 11.4 | 3.8 |
| WCM63 | 0 | 4 | Enzlautamide, radium223, | | 13.8 | 4.1 |
| | | | abiraterone, carboplatin | | | |
| | | | placlitaxel | | | |
| WCM73 | 0 | 0 | NA | | 11.2 | 3.8 |
| WCM78 | 0 | 1 | Alisertib | | 13.2 | 4 |
| WCM80 | 0 | 0 | NA | | 13.9 | 3.7 |
| WCM89 | 0 | 5 | abiraterone, enzalutamide, | | 13 | 3.9 |
| | | | radium223, docetaxel, | | | |
| | | | docetaxel carboplatin | | | |
| WCM90 | 1 | 0 | NA | | 12 | 2.2 |
| WCM91 | 0 | 1 | Docetaxel | | 10.3 | 3.5 |
| WCM119 | 1 | 1 | cabazitaxel | | 13.6 | 4 |
| WCM121 | 0 | 3 | docetaxel, abiraterone, | | 12.7 | 4.4 |
| | | | cabazitaxel | | | |
| WCM124 | 0 | 0 | NA | | 7.1 | 3.7 |
| WCM131 | 0 | 2 | leuprolide, enzalutamide | | 11.8 | 4 |
| WCM139 | 0 | 4 | MoAb J591, docetaxel | | 13 | NA |
| | | | cisplatin, carboplatin | | | |
| | | | paclitaxel, doxorubicin | | | |
| WCM142 | 0 | 2 | docetaxel, sipuleucel T | | 12.6 | 4 |
| WCM154 | 1 | 1 | Alisertib | | 10.2 | NA |
| WCM155 | 0 | 1 | Alisertib | | 10.7 | 3.1 |
| WCM156 | 1 | 1 | Alisertib | | 14.3 | 4.3 |
| WCM158 | 0 | 1 | enzalutamide | | 13 | 4.7 |
| WCM160 | 1 | 1 | docetaxel | | 10 | 4.1 |
| WCM161 | 1 | 0 | NA | | 11.8 | 3.9 |
| WCM162 | 0 | 3 | abiraterone, ipatasertib, | | 12.51 | 4 |
| | | | cabazitaxel | | | |
| WCM163 | 0 | 0 | NA | | 8.1 | 2.8 |
| WCM164 | 1 | 0 | NA | | 12 | 3.4 |
| WCM169 | 0 | 2 | carboplatin etoposide, | | 12.5 | 4 |
| | | | doxorubicin | | | |
| WCM170 | 0 | 5 | docetaxel, sipuleucel T, | | 11.4 | 3.6 |
| | | | indoximod, radium223, | | | |
| | | | carboplatin | | | |
| WCM172 | 1 | 3 | cabazitaxel, radium223, | | 11.6 | 3.7 |
| | | | carboplatin paclitaxel | | | |
| WCM176 | 0 | 1 | sipuleucel T | | 12.5 | 4.3 |
| WCM183 | 1 | 0 | NA | | 10.2 | 2.8 |
| WCM185 | 0 | 4 | enzalutamide, docetaxel, | | 9.5 | 3.4 |
| | | | 177Lu PSMA, | | | |
| | | | mitoxantrone | | | |
| WCM185-TP1 | 1 | 0 | NA | | 8 | 2.5 |
| WCM185-TP2 | 1 | 0 | NA | | 8.1 | 2.6 |
| WCM185-TP3 | 1 | 0 | NA | | 8.7 | 2.8 |
| WCM185-TP4 | 1 | 0 | NA | | 9.5 | 3 |
| WCM192 | 1 | 0 | NA | | NA | NA |
| WCM195 | 0 | 1 | carboplatin paclitaxel | | 8.7 | 2.8 |
| WCM198 | 1 | 0 | NA | | 11.8 | 3.8 |
| WCM199 | 0 | 0 | NA | | 11.9 | 4.1 |
| WCM201 | 0 | 1 | leuprolide orteronel | | 14 | 3.8 |
| WCM207 | 1 | 0 | NA | | NA | NA |
| WCM212 | 1 | 0 | NA | | 9.6 | 3 |
| WCM213 | 1 | 3 | enzalutamide, alisertib, | | 12.5 | 3.9 |
| | | | pazopanib | | | |
| WCM216 | 0 | 0 | NA | | 12.4 | 4.3 |
| WCM218 | 0 | 4 | docetaxel, enzalutamide, | | 13.7 | 4 |
| | | | carboplatin paclitaxel, | | | |
| | | | cabazitaxel | | | |
| WCM219 | 0 | 0 | NA | | 11.3 | 3.9 |
| WCM223 | 0 | 2 | degarelix, abiraterone | | 14.3 | 4 |
| WCM243 | 0 | 2 | docetaxel, radium223 | | 11.2 | 3.2 |
| WCM247 | 0 | NA | NA | | 12.9 | 3.7 |
| WCM252 | 1 | 0 | NA | | 10.5 | 3.4 |
| WCM253 | 1 | 1 | alisertib | | 11 | 3.2 |
| WCM261 | 1 | 0 | NA | | 10.4 | 3.8 |
| WCM341 | 0 | 1 | Alisertib | | NA | NA |
| WCM365 | 1 | 0 | NA | | 8.7 | 2.7 |
| WCM366 | NA | 1 | cabazitaxel carboplatin | | 14.7 | 4.2 |
| WCM369 | 0 | 1 | docetaxel | | 11.7 | 3.7 |
| WCM677 | 0 | 1 | radium223 | | 12 | 3.2 |
| WCM1388 | NA | 0 | NA | | NA | NA |
| | | | TESTOS- | | | |
| | ALP | LDH | TERONE | PSA | NSE | CGA |
| | (range | (range 98- | (range | (range 0- | (range | (range |
| | 32-91 | 192 | 175-781 | 4.0 ng/ | 3.7-8.9 | 0-95 |
| Sample | IU/L) | IU/L) | ng/dL) | mL) | ug/L) | ng/mL) |
| WCM0 | 114 | 349 | 136 | 0.03 | 44.2 | 405 |
| WCM1 | 1009 | 270 | 353 | 3056 | NA | 75 |
| WCM2 | 214 | 103 | NA | 2.64 | 9.4 | 222 |
| WCM4 | NA | 446 | 199 | 968.7 | NA | 48 |
| WCM6 | 54 | NA | 6 | 0.76 | 4 | 258 |
| WCM7 | 586 | 660 | 5.12 | 12.12 | 3.5 | 1129 |
| WCM9 | 230 | 186 | NA | 1.76 | NA | 84 |
| WCM10 | 38 | 177 | NA | 3 | 7.2 | 1379 |
| WCM12 | 77 | NA | NA | 0.01 | 8.8 | 319 |
| WCM14-TP1 | 821 | 1355 | 7 | 111.28 | NA | NA |
| WCM14-TP2 | 1326 | 1793 | 7 | 136.42 | NA | NA |
| WCM14-TP3 | 1033 | 754 | 7 | 138.14 | NA | NA |
| WCM14-TP4 | 782 | 1201 | 7 | 206.09 | NA | NA |
| WCM15 | 51 | 191 | 176 | 0.02 | 12.9 | 73 |
| WCM63 | 161 | 189 | NA | 43.63 | 7.5 | 60 |
| WCM73 | 51 | 180 | 409 | 356.1 | NA | NA |
| WCM78 | 161 | NA | 10 | 37.4 | NA | NA |
| WCM80 | 69 | 139 | 337 | 100.14 | 14.8 | 138 |
| WCM89 | 59 | 178 | 10 | 18.19 | 9 | 54 |
| WCM90 | 409 | 165 | 16 | 22.25 | 6.4 | 2937 |
| WCM91 | 69 | 162 | NA | 27.1 | NA | NA |
| WCM119 | 163 | 192 | 10 | 11.04 | 10.2 | 71 |
| WCM121 | 74 | 163 | NA | 16.8 | NA | NA |
| WCM124 | 304 | 242 | NA | 137 | 4.6 | 94 |
| WCM131 | 125 | 163 | NA | 0.46 | NA | 125 |
| WCM139 | NA | NA | NA | 31.14 | NA | 345 |
| WCM142 | 227 | 85 | NA | 14.33 | NA | NA |
| WCM154 | NA | 187 | 10 | 0.18 | 4.1 | 983 |
| WCM155 | 444 | 555 | NA | 1.29 | 61.2 | 1157 |
| WCM156 | 71 | 147 | NA | 1.83 | 23.6 | NA |
| WCM158 | 70 | 147 | NA | 0.49 | NA | NA |
| WCM160 | 109 | 116 | NA | 0.65 | 6.7 | 420 |
| WCM161 | 71 | 228 | 10 | 9.63 | 45.7 | 192 |
| WCM162 | 161 | 200 | 71 | 65.5 | 3.9 | 239 |
| WCM163 | 236 | 287 | NA | 110.9 | 35.6 | 17340 |
| WCM164 | 115 | 196 | 10 | 92.65 | 6.2 | 89 |
| WCM169 | 104 | 215 | 13 | 2.56 | 6.2 | 129 |
| WCM170 | 72 | 132 | NA | 5 | NA | NA |
| WCM172 | 92 | 106 | NA | 6.09 | NA | NA |
| WCM176 | 35 | 116 | 0.11 | 0.22 | NA | 49 |
| WCM183 | 114 | 178 | NA | 1272.5 | 12.7 | 362 |
| WCM185 | 91 | 212 | 10 | 542.5 | 3.3 | 148 |
| WCM185-TP1 | 147 | 187 | 7 | 1426 | NA | NA |
| WCM185-TP2 | 225 | NA | 7 | 2769 | NA | NA |
| WCM185-TP3 | 219 | NA | 7 | NA | NA | NA |
| WCM185-TP4 | 181 | 188 | 7 | 3124 | NA | NA |
| WCM192 | NA | NA | NA | NA | NA | NA |
| WCM195 | 96 | 364 | NA | 14.72 | 2.4 | 333 |
| WCM198 | 60 | 173 | 12 | 0.03 | 7.2 | 115 |
| WCM199 | 43 | 180 | NA | 0.45 | NA | NA |
| WCM201 | 53 | 161 | NA | 2.46 | NA | NA |
| WCM207 | NA | 153 | 10 | 0.13 | 6.6 | 266 |
| WCM212 | 135 | 1047 | 10 | 29.94 | 370 | 841 |
| WCM213 | 42 | 137 | NA | 2.2 | 10.4 | 78 |
| WCM216 | 81 | 170 | NA | 56.8 | 5.6 | 627 |
| WCM218 | 83 | 257 | 10 | 6.55 | 6 | 107 |
| WCM219 | 55 | 135 | 36 | 44.91 | NA | NA |
| WCM223 | 59 | 149 | NA | 7527 | NA | NA |
| WCM243 | 129 | 187 | NA | 0.58 | 5.8 | 170 |
| WCM247 | 115 | 198 | 287 | 0.66 | 3.4 | 104 |
| WCM252 | 66 | 191 | NA | 0.01 | 80.2 | 602 |
| WCM253 | 318 | 360 | 10 | 44 | 31.9 | 238 |
| WCM261 | 86 | 144 | 10 | 1.4 | NA | NA |
| WCM341 | NA | 24.27 | 10 | NA | NA | 1221 |
| WCM365 | 178 | 155 | 10 | 668.6 | 3.3 | 242 |
| WCM366 | 116 | 153 | 303 | 14.6 | 4.9 | 119 |
| WCM369 | 142 | 211 | 21 | 78 | NA | NA |
| WCM677 | 80 | 141 | 10 | 26 | 67.1 | 214 |
| WCM1388 | NA | NA | NA | NA | NA | NA |
| | | | | TOTAL | Nadir | |
| | | | | PSA before | PSA/ | |
| | CEA | Date of | | starting | PSA at | |
| | (range <= | death or | | therapy | 12 weeks | PSA |
| | 3.0 | last | alive = 0, | (range 0-4.0 | if no | change |
| Sample | mg/mL) | follow up | dead = 1 | ng/mL) | decline | (%) |
| WCM0 | 992.8 | Jan. 9, 2013 | 1 | 0.03 | 0.03 | 0 |
| WCM1 | 8.1 | Jun. 30, 2016 | 1 | 3056 | 344 | −89 |
| WCM2 | NA | Feb. 15, 2014 | 1 | 2.64 | 0.31 | −88 |
| WCM4 | NA | Mar. 6, 2015 | 1 | 968.7 | 277.9 | −71 |
| WCM6 | 2.6 | Feb. 9, 2016 | 1 | 0.76 | 0.71 | −7 |
| WCM7 | 2.4 | Sep. 23, 2013 | 0 | 12.12 | 33.8 | 179 |
| WCM9 | NA | Sep. 21, 2015 | 1 | 1.76 | 1.54 | −13 |
| WCM10 | NA | Apr. 3, 2017 | 1 | 3 | 4.1 | 37 |
| WCM12 | 2 | Jul. 29, 2015 | 1 | NA | NA | NA |
| WCM14-TP1 | NA | Aug. 24, 2017 | 1 | 111.18 | 206.09 | 85 |
| WCM14-TP2 | NA | Aug. 24, 2017 | 1 | 111.18 | 206.09 | 85 |
| WCM14-TP3 | NA | Aug. 24, 2017 | 1 | 111.18 | 206.09 | 85 |
| WCM14-TP4 | NA | Aug. 24, 2017 | 1 | 111.18 | 206.09 | 85 |
| WCM15 | 74.2 | Oct. 6, 2015 | 1 | 0.02 | 0.02 | 0 |
| WCM63 | NA | Jan. 11, 2018 | 1 | 265.9 | 2.48 | −99 |
| WCM73 | NA | Sep. 11, 2014 | 0 | 356.1 | 3.7 | −99 |
| WCM78 | NA | Mar. 3, 2014 | 1 | 37.4 | 182.9 | 389 |
| WCM80 | NA | Jan. 11, 2018 | 0 | 100.14 | 0.35 | −100 |
| WCM89 | NA | Aug. 13, 2017 | 1 | 22.25 | 7.85 | −65 |
| WCM90 | NA | Oct. 9, 2013 | 1 | NA | NA | NA |
| WCM91 | NA | Dec. 1, 2014 | 1 | 27.1 | 74.6 | 175 |
| WCM119 | NA | May 12, 2014 | 1 | 11.04 | 11.19 | 1 |
| WCM121 | NA | Jun. 7, 2014 | 1 | 11.02 | 39.36 | 257 |
| WCM124 | NA | Jun. 19, 2015 | 1 | 137 | 202 | 47 |
| WCM131 | NA | Jan. 23, 2015 | 1 | 0.46 | 0.19 | −59 |
| WCM139 | NA | Oct. 17, 2014 | 1 | 10.2 | 31.14 | 205 |
| WCM142 | NA | Nov. 27, 2014 | 1 | 14.33 | 61.44 | 329 |
| WCM154 | NA | Jun. 19, 2014 | 1 | 0.18 | 0.16 | −11 |
| WCM155 | 1287 | Apr. 3, 2015 | 1 | 1.29 | 0.88 | −32 |
| WCM156 | NA | Oct. 12, 2014 | 1 | 1.83 | 0.13 | −93 |
| WCM158 | NA | Jan. 3, 2018 | 0 | 0.74 | 1.01 | 36 |
| WCM160 | NA | Jun. 29, 2016 | 0 | 18.28 | 31.18 | 71 |
| WCM161 | NA | Aug. 16, 2014 | 1 | 26.84 | 80.39 | 200 |
| WCM162 | NA | Dec. 12, 2014 | 1 | 65.5 | 7.73 | −88 |
| WCM163 | NA | Mar. 24, 2014 | 1 | 110.9 | 210.6 | 90 |
| WCM164 | NA | Aug. 7, 2014 | 1 | 92.65 | 213.3 | 130 |
| WCM169 | NA | Oct. 7, 2015 | 1 | 12.29 | 9.94 | −19 |
| WCM170 | NA | May 4, 2017 | 0 | 6.8 | 0.62 | −91 |
| WCM172 | NA | May 24, 2016 | 1 | 59.38 | 3.22 | −95 |
| WCM176 | NA | Dec. 18, 2017 | 0 | 0.22 | 0.21 | −5 |
| WCM183 | NA | Mar. 7, 2015 | 1 | 1272.5 | 3243 | 155 |
| WCM185 | NA | Dec. 14, 2017 | 1 | 542.5 | 562 | 4 |
| WCM185-TP1 | NA | Dec. 14, 2017 | 1 | 1426 | 3124 | 119 |
| WCM185-TP2 | NA | Dec. 14, 2017 | 1 | 2769 | 3124 | 13 |
| WCM185-TP3 | NA | Dec. 14, 2017 | 1 | 2769 | 3124 | 13 |
| WCM185-TP4 | NA | Dec. 14, 2017 | 1 | 2769 | 3124 | 13 |
| WCM192 | NA | Jul. 25, 2015 | 1 | NA | NA | NA |
| WCM195 | NA | Jul. 24, 2014 | 1 | 14.72 | 45.12 | 207 |
| WCM198 | 21.8 | Feb. 28, 2014 | 0 | 0.03 | 0.03 | 0 |
| WCM199 | NA | May 1, 2014 | 1 | 0.45 | 0.75 | 67 |
| WCM201 | NA | Jan. 11, 2018 | 0 | NA | NA | NA |
| WCM207 | NA | Aug. 31, 2014 | 1 | 0.13 | 0.83 | 538 |
| WCM212 | NA | May 14, 2014 | 0 | NA | NA | NA |
| WCM213 | NA | Apr. 19, 2016 | 1 | 2.67 | 0.6 | −78 |
| WCM216 | NA | Jan. 11, 2018 | 0 | 56.8 | 0.06 | −100 |
| WCM218 | NA | Jul. 30, 2016 | 1 | 44.93 | 32.33 | −28 |
| WCM219 | NA | May 1, 2017 | 0 | 44.91 | 6.18 | −86 |
| WCM223 | NA | Jan. 25, 2018 | 0 | 7527 | 58 | −99 |
| WCM243 | 2.9 | Jan. 11, 2016 | 1 | 0.58 | 0.33 | −43 |
| WCM247 | NA | Feb. 22, 2016 | 1 | 0.66 | 0.01 | −98 |
| WCM252 | 26 | Dec. 18, 2014 | 1 | 0.01 | 0.01 | 0 |
| WCM253 | NA | Mar. 18, 2015 | 1 | 36 | 3.68 | −90 |
| WCM261 | NA | Jan. 18, 2018 | 0 | 1.4 | 0.12 | −91 |
| WCM341 | NA | Feb. 18, 2015 | 0 | 24.27 | 35.69 | 47 |
| WCM365 | NA | Apr. 24, 2015 | 1 | NA | NA | NA |
| WCM366 | NA | Aug. 2, 2016 | 1 | 14.6 | 1.32 | −91 |
| WCM369 | NA | Jan. 29, 2017 | 1 | 78 | 1.07 | −99 |
| WCM677 | 4.1 | Apr. 12, 2016 | 1 | 26 | 0.22 | −99 |
| WCM1388 | NA | Feb. 18, 2018 | 0 | NA | NA | NA |

TABLE 11
Plasma and tumor tissue samples genomic data

| | | | | Tumor | | | | Missense | | |
|---|---|---|---|---|---|---|---|---|---|---|
| | | | | content | | | SNVs | SNVs | | Label in main |
| Sample | Class | Date | Type | (TC) | Ploidy | CNAF | load | load | Site | figures |
| WCM0 | CRPC- | Jan. 3, 2013 | Plasma | 0.51 | 1.89 | 0.406233 | 174 | 47 | NA | WCM0_Plasma |
| | NE | | | | | | | | | |
| WCM10 | CRPC- | Apr. 24, 2013 | Plasma | 0.18 | 2 | 0.002196 | 23 | 11 | NA | NA |
| | NE | | | | | | | | | |
| WCM119 | CRPC- | Nov. 21, 2013 | Plasma | 0.67 | 1.84 | 0.522444 | 93 | 24 | NA | NA |
| | Adeno | | | | | | | | | |
| WCM1 | HNPC | Feb. 6, 2013 | Plasma | 0.34 | 1.97 | 0.34136 | 77 | 34 | NA | NA |
| WCM121 | CRPC- | Oct. 16, 2013 | Plasma | NA | 1.98 | NA | 15 | 6 | NA | NA |
| | Adeno | | | | | | | | | |
| WCM12 | CRPC- | Jun. 4, 2013 | Plasma | 0.1 | 1.99 | 0.084694 | 8 | 2 | NA | NA |
| | NE | | | | | | | | | |
| WCM124 | CRPC- | Oct. 18, 2013 | Plasma | NA | 1.96 | NA | 9 | 1 | NA | NA |
| | Adeno | | | | | | | | | |
| WCM131 | HNPC | Dec. 3, 2013 | Plasma | NA | NA | NA | 553 | 152 | NA | NA |
| WCM1388 | CRPC- | Aug. 1, 2017 | Plasma | 0.94 | 1.73 | 0.452772 | NA | NA | NA | NA |
| | NE | | | | | | | | | |
| WCM139 | CRPC- | Dec. 4, 2013 | Plasma | 0.07 | 1.98 | 0.212997 | 20 | 11 | NA | NA |
| | Adeno | | | | | | | | | |
| WCM142 | HNPC | Dec. 19, 2013 | Plasma | 0.08 | 1.99 | 0.457222 | 34 | 13 | NA | NA |
| WCM14-TP1 | CRPC- | Apr. 26, 2017 | Plasma | 0.63 | 3.3 | 0.914505 | 99 | 39 | NA | WCM14_Plasma1 |
| | Adeno | | | | | | | | | |
| WCM14-TP2 | CRPC- | May 10, 2017 | Plasma | 0.58 | 3.1 | 0.883492 | 114 | 42 | NA | WCM14_Plasma2 |
| | Adeno | | | | | | | | | |
| WCM14-TP3 | CRPC- | Jun. 7, 2017 | Plasma | 0.3 | 2.63 | 0.900793 | 71 | 27 | NA | WCM14_Plasma3 |
| | Adeno | | | | | | | | | |
| WCM14-TP4 | CRPC- | Jun. 28, 2017 | Plasma | 0.62 | 3.1 | 0.862413 | 87 | 32 | NA | WCM14_Plasma4 |
| | Adeno | | | | | | | | | |
| WCM15 | CRPC- | Jun. 11, 2013 | Plasma | 0.13 | 2.1 | 0.777127 | 38 | 17 | NA | NA |
| | NE | | | | | | | | | |
| WCM154 | CRPC- | Jan. 10, 2014 | Plasma | 0.4 | 2.6 | 0.742805 | 91 | 36 | NA | NA |
| | NE | | | | | | | | | |
| WCM155 | CRPC- | Jan. 8, 2014 | Plasma | 0.75 | 2.81 | 0.815751 | 106 | 34 | NA | NA |
| | NE | | | | | | | | | |
| WCM156 | CRPC- | Dec. 19, 2013 | Plasma | 0.1 | 2.02 | 0.36514 | 48 | 20 | NA | NA |
| | Adeno | | | | | | | | | |
| WCM158 | CRPC- | Jan. 28, 2014 | Plasma | 0.22 | 1.94 | 0.118501 | 22 | 12 | NA | NA |
| | Adeno | | | | | | | | | |
| WCM160 | CRPC- | Feb. 12, 2014 | Plasma | 0.16 | 2 | 0.518303 | 47 | 14 | NA | NA |
| | NE | | | | | | | | | |
| WCM161 | CRPC- | Feb. 13, 2014 | Plasma | 0.51 | 2.02 | 0.43724 | 50 | 14 | NA | WCM161_Plasma |
| | Adeno | | | | | | | | | |
| WCM162 | CRPC- | Feb. 18, 2014 | Plasma | 0.57 | 2.27 | 0.355735 | 113 | 30 | NA | NA |
| | Adeno | | | | | | | | | |
| WCM163 | CRPC- | Mar. 5, 2014 | Plasma | 0.82 | 2.8 | 0.844383 | 90 | 38 | NA | NA |
| | NE | | | | | | | | | |
| WCM164 | CRPC- | Feb. 18, 2014 | Plasma | 0.07 | 2.03 | 0.499529 | 37 | 14 | NA | NA |
| | Adeno | | | | | | | | | |
| WCM169 | CRPC- | Apr. 15, 2014 | Plasma | 0.06 | 2 | 0.24227 | 22 | 8 | NA | NA |
| | Adeno | | | | | | | | | |
| WCM170 | CRPC- | Mar. 20, 2014 | Plasma | 0.07 | 2.04 | 0.581636 | 20 | 4 | NA | NA |
| | Adeno | | | | | | | | | |
| WCM172 | CRPC- | Mar. 18, 2014 | Plasma | NA | 1.98 | NA | 21 | 9 | NA | NA |
| | Adeno | | | | | | | | | |
| WCM176 | HNPC | Dec. 15, 2014 | Plasma | NA | 1.95 | NA | 60 | 29 | NA | NA |
| WCM183 | CRPC- | Apr. 15, 2014 | Plasma | 0.62 | 2.9 | 0.857458 | 419 | 141 | NA | NA |
| | Adeno | | | | | | | | | |
| WCM185 | CRPC- | Apr. 30, 2014 | Plasma | NA | 2.04 | NA | 30 | 6 | NA | WCM185_Plasma |
| | Adeno | | | | | | | | | |
| WCM185-TP1 | CRPC- | May 17, 2017 | Plasma | 0.17 | 2.17 | 0.219132 | 75 | 17 | NA | WCM185_Plasma1 |
| | Adeno | | | | | | | | | |
| WCM185-TP2 | CRPC- | May 24, 2017 | Plasma | 0.76 | 2.11 | 0.212067 | 90 | 28 | NA | WCM185_Plasma2 |
| | Adeno | | | | | | | | | |
| WCM185-TP3 | CRPC- | May 31, 2017 | Plasma | 0.74 | 2.08 | 0.166852 | 88 | 26 | NA | WCM185_Plasma3 |
| | Adeno | | | | | | | | | |
| WCM185-TP4 | CRPC- | Jun. 13, 2017 | Plasma | NA | 2.11 | NA | 46 | 14 | NA | WCM185_Plasma4 |
| | Adeno | | | | | | | | | |
| WCM192 | CRPC- | Feb. 5, 2015 | Plasma | NA | 2.02 | NA | 36 | 12 | NA | NA |
| | NE | | | | | | | | | |
| WCM195 | CRPC- | Apr. 29, 2014 | Plasma | 0.08 | 1.99 | 0.222119 | 13 | 5 | NA | NA |
| | Adeno | | | | | | | | | |
| WCM198 | CRPC- | Nov. 26, 2013 | Plasma | 0.52 | 2.19 | 0.377376 | 150 | 49 | NA | NA |
| | NE | | | | | | | | | |
| WCM199 | CRPC- | Dec. 23, 2013 | Plasma | NA | 1.97 | NA | 19 | 5 | NA | NA |
| | Adeno | | | | | | | | | |
| WCM201 | HNPC | May 12, 2014 | Plasma | 0.18 | 1.99 | 0.073614 | 12 | 3 | NA | NA |
| WCM207 | CRPC- | May 7, 2014 | Plasma | 0.7 | 2.9 | 0.695142 | 86 | 29 | NA | NA |
| | NE | | | | | | | | | |
| WCM212 | CRPC- | May 12, 2014 | Plasma | 0.58 | 2.7 | 0.761776 | 93 | 20 | NA | NA |
| | NE | | | | | | | | | |
| WCM213 | CRPC- | May 20, 2014 | Plasma | 0.16 | 1.98 | 0.220748 | 32 | 11 | NA | NA |
| | Adeno | | | | | | | | | |
| WCM216 | CRPC- | Jul. 3, 2014 | Plasma | 0.48 | 1.97 | 3.10E−05 | 11 | 2 | NA | NA |
| | Adeno | | | | | | | | | |
| WCM218 | CRPC- | Sep. 16, 2014 | Plasma | 0.13 | 1.99 | 0.07904 | 12 | 3 | NA | NA |
| | Adeno | | | | | | | | | |
| WCM219 | CRPC- | Jul. 8, 2014 | Plasma | 0.05 | 1.98 | 0.313314 | 21 | 9 | NA | NA |
| | Adeno | | | | | | | | | |
| WCM2 | CRPC- | Feb. 21, 2013 | Plasma | 0.12 | 2.01 | 0.439123 | 41 | 12 | NA | NA |
| | Adeno | | | | | | | | | |
| WCM223 | CRPC- | Nov. 25, 2014 | Plasma | 0.12 | 2 | 0.072258 | 14 | 1 | NA | NA |
| | Adeno | | | | | | | | | |
| WCM243 | CRPC- | Aug. 20, 2014 | Plasma | 0.2 | 2.06 | 0.610595 | 33 | 13 | NA | NA |
| | NE | | | | | | | | | |
| WCM247 | HNPC | Sep. 11, 2014 | Plasma | 0.05 | 2.04 | 0.672381 | 32 | 8 | NA | NA |
| WCM252 | CRPC- | Sep. 3, 2014 | Plasma | 0.72 | 1.98 | 0.215364 | 397 | 55 | NA | NA |
| | NE | | | | | | | | | |
| WCM253 | CRPC- | Oct. 22, 2014 | Plasma | 0.08 | 2.03 | 0.090601 | 43 | 9 | NA | NA |
| | NE | | | | | | | | | |
| WCM261 | CRPC- | Oct. 28, 2014 | Plasma | NA | 1.98 | NA | 7 | 0 | NA | NA |
| | Adeno | | | | | | | | | |
| WCM341 | CRPC- | Nov. 17, 2014 | Plasma | 0.29 | 2.6 | 0.923577 | 77 | 25 | NA | NA |
| | Adeno | | | | | | | | | |
| WCM365 | CRPC- | Mar. 18, 2015 | Plasma | 0.58 | 2.68 | 0.774055 | 73 | 22 | NA | NA |
| | Adeno | | | | | | | | | |
| WCM366 | HNPC | Mar. 26, 2015 | Plasma | 0.04 | 1.96 | 0.36879 | 16 | 8 | NA | NA |
| WCM369 | CRPC- | Jun. 2, 2015 | Plasma | 0.07 | 1.96 | 0.031945 | 10 | 5 | NA | NA |
| | Adeno | | | | | | | | | |
| WCM4 | HNPC | Mar. 28, 2013 | Plasma | NA | 2.04 | NA | 50 | 18 | NA | NA |
| WCM6 | CRPC- | Mar. 28, 2013 | Plasma | NA | 2.04 | NA | 28 | 7 | NA | NA |
| | Adeno | | | | | | | | | |
| WCM63 | CRPC- | Sep. 20, 2013 | Plasma | 0.15 | 2.01 | 0.363378 | 24 | 8 | NA | NA |
| | Adeno | | | | | | | | | |
| WCM677 | CRPC- | Dec. 22, 2015 | Plasma | 0.53 | 1.95 | 0.32031 | 91 | 23 | NA | NA |
| | NE | | | | | | | | | |
| WCM7 | CRPC- | Apr. 9, 2013 | Plasma | 0.22 | 2 | 0.377302 | 36 | 12 | NA | NA |
| | Adeno | | | | | | | | | |
| WCM73 | HNPC | Aug. 28, 2013 | Plasma | 0.03 | 2.01 | 0.587287 | 53 | 14 | NA | NA |
| WCM78 | CRPC- | Nov. 12, 2013 | Plasma | 0.18 | 2.02 | 0.142563 | 56 | 23 | NA | NA |
| | Adeno | | | | | | | | | |
| WCM80 | HNPC | Sep. 26, 2013 | Plasma | 0.53 | 1.95 | 0.099424 | 86 | 31 | NA | NA |
| WCM89 | CRPC- | Sep. 24, 2013 | Plasma | NA | 1.97 | NA | 19 | 8 | NA | NA |
| | Adeno | | | | | | | | | |
| WCM90 | CRPC- | Oct. 8, 2013 | Plasma | 0.32 | 2.01 | 0.26617 | 42 | 16 | NA | NA |
| | Adeno | | | | | | | | | |
| WCM91 | CRPC- | Sep. 3, 2014 | Plasma | NA | 2.04 | NA | 34 | 8 | NA | NA |
| | Adeno | | | | | | | | | |
| WCM9 | CRPC- | Apr. 29, 2013 | Plasma | 0.25 | 1.99 | 0.006078 | 22 | 7 | NA | NA |
| | Adeno | | | | | | | | | |
| WCM0_LM1 | CRPC- | Jan. 9, 2013 | Tissue | 0.95 | 1.72 | 0.497432 | 161 | 77 | Liver | WCM0_Liverl |
| | NE | | | | | | | | | |
| WCM0_LM2 | CRPC- | Jan. 9, 2013 | Tissue | 0.96 | 1.63 | 0.518523 | 177 | 90 | Liver | WCM0_Liver2 |
| | NE | | | | | | | | | |
| WCM0_PM1 | CRPC- | Jan. 9, 2013 | Tissue | 0.96 | 1.63 | 0.431888 | 117 | 47 | PelvicMass | WCM0_PM1 |
| | NE | | | | | | | | | |
| WCM0_PM2 | CRPC- | Jan. 9, 2013 | Tissue | 0.98 | 2.07 | 0.717882 | 167 | 69 | PelvicMass | WCM0_PM2 |
| | NE | | | | | | | | | |
| WCM0_T24 | CRPC- | Jan. 9, 2013 | Tissue | 0.92 | 1.66 | 0.424753 | 144 | 62 | Lymphnode | WCM0_LN2 |
| | NE | | | | | | | | | |
| WCM0_T5 | CRPC- | Jan. 9, 2013 | Tissue | 0.98 | 1.7 | 0.443587 | 169 | 78 | Lymphnode | WCM0_LN1 |
| | NE | | | | | | | | | |
| WCM10_Z1-1 | CRPC- | Apr. 24, 2013 | Tissue | 0.64 | 1.98 | 0.034373 | 1345 | 644 | Lymphnode | NA |
| | NE | | | | | | | | | |
| WCM119_Z6_1 | CRPC- | Oct. 15, 2013 | Tissue | 0.7 | 3.2 | 0.875076 | 188 | 104 | Lymphnode | NA |
| | Adeno | | | | | | | | | |
| WCM121_Z1_1 | CRPC- | Oct. 18, 2013 | Tissue | 0.42 | 2.7 | 0.890388 | 347 | 178 | Lymphnode | NA |
| | Adeno | | | | | | | | | |
| WCM124_X1_3 | CRPC- | Oct. 21, 2013 | Tissue | 0.44 | 3 | 0.923381 | 247 | 154 | Bone | NA |
| | Adeno | | | | | | | | | |
| WCM12_Z10_1 | HNPC | Jan. 25, 2012 | Tissue | 0.8 | 2.06 | 0.251133 | 490 | 289 | Prostate | NA |
| WCM131_Z1_1 | CRPC- | Nov. 21, 2014 | Tissue | NA | NA | NA | 1212 | 614 | Bone | NA |
| | Adeno | | | | | | | | | |
| WCM131_Z2_1 | HNPC | Oct. 29, 2013 | Tissue | NA | NA | NA | 870 | 466 | Bone | NA |
| WCM1388_Z3_1 | CRPC- | Jul. 28, 2017 | Tissue | 0.93 | 1.81 | 0.467737 | 236 | 140 | Liver | NA |
| | NE | | | | | | | | | |
| WCM139_Z3_1 | CRPC- | Dec. 4, 2013 | Tissue | 0.81 | 2.34 | 0.35712 | 179 | 113 | Bone | NA |
| | Adeno | | | | | | | | | |
| WCM14_x1_1 | CRPC- | Nov. 26, 2012 | Tissue | 0.41 | 2.83 | 0.875067 | 52 | 29 | Prostate | WCM14_TURP |
| | Adeno | | | | | | | | | |
| WCM14_Z11_1 | CRPC- | Jul. 19, 2017 | Tissue | 0.7 | 3 | 0.870726 | 624 | 372 | Liver | WCM14_Liver |
| | Adeno | | | | | | | | | |
| WCM154_Z1_1 | CRPC- | Jan. 7, 2014 | Tissue | 0.85 | 3.4 | 0.805325 | 178 | 103 | Bone | NA |
| | NE | | | | | | | | | |
| WCM155_Z1_1 | CRPC- | Jan. 8, 2014 | Tissue | 0.97 | 3.15 | 0.813507 | 159 | 88 | Liver | NA |
| | NE | | | | | | | | | |
| WCM155_Z2_1 | CRPC- | Jan. 31, 2015 | Tissue | 0.71 | 2.96 | 0.80734 | 312 | 178 | GallBladder | NA |
| | NE | | | | | | | | | |
| WCM156_Z5_1 | CRPC- | Nov. 20, 2013 | Tissue | 0.52 | 2.06 | 0.254913 | 202 | 131 | Prostate | NA |
| | Adeno | | | | | | | | | |
| WCM158_X1_1 | CRPC- | Jan. 30, 2014 | Tissue | 0.3 | 2.5 | 0.899495 | 182 | 115 | Bone | NA |
| | Adeno | | | | | | | | | |
| WCM15_Z1_1 | CRPC- | Jun. 11, 2013 | Tissue | 0.94 | 1.9 | 0.51896 | 112 | 54 | Lymphnode | NA |
| | NE | | | | | | | | | |
| WCM160_X1_1 | CRPC- | Feb. 20, 2014 | Tissue | NA | 2 | NA | 69 | 43 | Bone | NA |
| | NE | | | | | | | | | |
| WCM160_Z7_1 | CRPC- | Nov. 4, 2014 | Tissue | 0.37 | 2 | 0.398985 | 145 | 85 | Bone | NA |
| | Adeno | | | | | | | | | |
| WCM161_Z1_1 | CRPC- | Feb. 13, 2014 | Tissue | 0.74 | 3.17 | 0.876004 | 150 | 102 | Bone | WCM161_Bone |
| | Adeno | | | | | | | | | |
| WCM161_Z15_1 | CRPC- | May 15, 2014 | Tissue | 0.96 | 2.84 | 0.768569 | 152 | 86 | Liver | WCM161_Liver |
| | NE | | | | | | | | | |
| WCM161_Z6_1 | CRPC- | Feb. 13, 2014 | Tissue | 0.4 | 3 | 0.962918 | 142 | 88 | Lymphnode | WCM161_Lymphnode |
| | Adeno | | | | | | | | | |
| WCM162_Z1_1 | CRPC- | Feb. 14, 2014 | Tissue | 0.82 | 2.5 | 0.439627 | 218 | 129 | Lung | NA |
| | Adeno | | | | | | | | | |
| WCM162_Z9_1 | CRPC- | Jul. 1, 2014 | Tissue | NA | 1.99 | NA | 146 | 91 | Bone | NA |
| | Adeno | | | | | | | | | |
| WCM163_Z7_1 | CRPC- | Feb. 14, 2014 | Tissue | 0.92 | 3.05 | 0.790792 | 160 | 99 | Liver | NA |
| | NE | | | | | | | | | |
| WCM164_X1_1 | CRPC- | Feb. 19, 2014 | Tissue | 0.5 | 2.9 | 0.851095 | 138 | 82 | Bone | NA |
| | Adeno | | | | | | | | | |
| WCM169_X1_1 | CRPC- | Jun. 30, 2015 | Tissue | 0.1 | 2.06 | 0.51887 | 250 | 164 | Bone | NA |
| | Adeno | | | | | | | | | |
| WCM170_Z1_1 | CRPC- | Mar. 12, 2014 | Tissue | 0.37 | 2.07 | 0.375831 | 145 | 92 | Bone | NA |
| | Adeno | | | | | | | | | |
| WCM170_Z16_1 | CRPC- | Mar. 17, 2017 | Tissue | 0.78 | 2.09 | 0.384168 | 214 | 121 | SpinalCord | NA |
| | Adeno | | | | | | | | | |
| WCM172_X1_1 | CRPC- | Mar. 19, 2014 | Tissue | 0.39 | 2.7 | 0.810411 | 161 | 101 | Bone | NA |
| | Adeno | | | | | | | | | |
| WCM172_X2_1 | CRPC- | Mar. 5, 2015 | Tissue | 0.8 | 3 | 0.8253 | 171 | 96 | Bone | NA |
| | Adeno | | | | | | | | | |
| WCM176_Z1_1 | HNPC | Mar. 26, 2014 | Tissue | 0.83 | 1.93 | 0.101837 | 127 | 71 | Lung | NA |
| WCM183_Z4_1 | CRPC- | Apr. 2, 2014 | Tissue | 0.96 | 3.7 | 0.855027 | 377 | 229 | Liver | NA |
| | Adeno | | | | | | | | | |
| WCM185_X1_1 | CRPC- | Jul. 1, 2015 | Tissue | 0.14 | 2.04 | 0.574249 | 262 | 177 | Prostate | WCM185_TURP |
| | Adeno | | | | | | | | | |
| WCM185_Z1_1 | CRPC- | Apr. 8, 2014 | Tissue | 0.81 | 2.01 | 0.137001 | 117 | 64 | Spine | WCM185_Spine |
| | Adeno | | | | | | | | | |
| WCM192_X1_1 | CRPC- | Jan. 26, 2015 | Tissue | 0.83 | 1.96 | 0.274942 | 346 | 187 | Lymphnode | NA |
| | NE | | | | | | | | | |
| WCM192_Z1_1 | CRPC- | Apr. 4, 2014 | Tissue | 0.38 | 2.5 | 0.748843 | 150 | 76 | Prostate | NA |
| | NE | | | | | | | | | |
| WCM195_Z8_1 | CRPC- | Apr. 10, 2014 | Tissue | 0.95 | 3.2 | 0.908459 | 160 | 90 | Bladder | NA |
| | Adeno | | | | | | | | | |
| WCM198_Z1_1 | CRPC- | Nov. 22, 2013 | Tissue | 0.98 | 2.36 | 0.374634 | 156 | 97 | Lymphnode | NA |
| | NE | | | | | | | | | |
| WCM199_Z3_1 | CRPC- | Dec. 5, 2013 | Tissue | 0.83 | 2.55 | 0.502945 | 125 | 68 | Liver | NA |
| | Adeno | | | | | | | | | |
| WCM1_T | HNPC | Feb. 15, 2013 | Tissue | NA | 2.01 | NA | 58 | 15 | Bone | NA |
| WCM201_Z1_1 | CRPC- | Oct. 7, 2013 | Tissue | 0.64 | 3.1 | 0.942948 | 165 | 97 | Lymphnode | NA |
| | Adeno | | | | | | | | | |
| WCM207_X1_1 | CRPC- | May 7, 2014 | Tissue | 0.99 | 3.2 | 0.676731 | 173 | 110 | Liver | NA |
| | NE | | | | | | | | | |
| WCM212_Z2_1 | CRPC- | May 12, 2014 | Tissue | 0.91 | 3.06 | 0.746506 | 152 | 84 | Softtissue | NA |
| | NE | | | | | | | | | |
| WCM213_X1_1 | CRPC- | Mar. 11, 2015 | Tissue | NA | 2.6 | NA | 293 | 186 | Liver | NA |
| | Adeno | | | | | | | | | |
| WCM213_Z13_1 | CRPC- | Jun. 4, 2014 | Tissue | NA | 2.06 | NA | 264 | 163 | Liver | NA |
| | Adeno | | | | | | | | | |
| WCM216_Z1_1 | CRPC- | Jun. 16, 2014 | Tissue | NA | 2.01 | NA | 143 | 88 | Bone | NA |
| | Adeno | | | | | | | | | |
| WCM218_Z11_1 | CRPC- | Apr. 8, 2015 | Tissue | 0.45 | 2.05 | 0.298643 | 216 | 129 | Bone | NA |
| | Adeno | | | | | | | | | |
| WCM218_Z3_1 | CRPC- | Jul. 23, 2014 | Tissue | 0.55 | 2.05 | 0.282554 | 210 | 131 | Bone | NA |
| | Adeno | | | | | | | | | |
| WCM223_Z2_1 | CRPC- | Jun. 9, 2014 | Tissue | 0.89 | 1.91 | 0.073906 | 243 | 145 | Prostate | NA |
| | Adeno | | | | | | | | | |
| WCM223_Z4_1 | CRPC- | Dec. 22, 2016 | Tissue | 0.94 | 1.99 | 0.079075 | 251 | 141 | Brain | NA |
| | Adeno | | | | | | | | | |
| WCM243_X1_1 | CRPC- | Aug. 14, 2014 | Tissue | 0.63 | 2.75 | 0.696385 | 197 | 110 | Bone | NA |
| | NE | | | | | | | | | |
| WCM247_Z9_1 | CRPC- | Aug. 25, 2014 | Tissue | 0.45 | 1.83 | 0.641502 | 136 | 84 | Bone | NA |
| | Adeno | | | | | | | | | |
| WCM252_Z3_1 | CRPC- | Sep. 3, 2014 | Tissue | 0.73 | 2.01 | 0.214638 | 174 | 84 | Liver | NA |
| | NE | | | | | | | | | |
| WCM253_Z1_1 | CRPC- | Sep. 12, 2014 | Tissue | 0.59 | 2.9 | 0.766665 | 374 | 235 | Liver | NA |
| | NE | | | | | | | | | |
| WCM261_Z4_1 | CRPC- | Oct. 14, 2014 | Tissue | 0.47 | 2.7 | 0.770169 | 190 | 113 | Bone | NA |
| | Adeno | | | | | | | | | |
| WCM2_Z3_1 | CRPC- | Feb. 19, 2013 | Tissue | 0.43 | 2.23 | 0.641158 | 204 | 120 | Lymphnode | NA |
| | Adeno | | | | | | | | | |
| WCM341_X1_1_p9 | CRPC- | N/A | Tissue | 0.49 | 1.7 | 0.737809 | 136 | 84 | Lymphnode | NA |
| | Adeno | | | | | | | | | |
| WCM341_Z2_1 | CRPC- | N/A | Tissue | 0.74 | 1.8 | 0.473458 | 175 | 104 | Lymphnode | NA |
| | Adeno | | | | | | | | | |
| WCM365_Z2_1 | CRPC- | Mar. 18, 2015 | Tissue | 0.77 | 2.99 | 0.74207 | 231 | 138 | Skin | NA |
| | Adeno | | | | | | | | | |
| WCM366_X1_1 | CRPC- | Mar. 11, 2015 | Tissue | 0.68 | 1.9 | 0.144074 | 163 | 85 | Lymphnode | NA |
| | Adeno | | | | | | | | | |
| WCM369_Z2_1 | CRPC- | Apr. 2, 2015 | Tissue | 0.23 | 1.98 | 0.226305 | 133 | 81 | Bone | NA |
| | Adeno | | | | | | | | | |
| WCM4_X1_1 | CRPC- | Apr. 30, 2014 | Tissue | 0.24 | 2.35 | 0.764132 | 210 | 124 | Bone | NA |
| | Adeno | | | | | | | | | |
| WCM63_X1_1 | CRPC- | Mar. 6, 2015 | Tissue | NA | 1.96 | NA | 100 | 62 | Bone | NA |
| | Adeno | | | | | | | | | |
| WCM63_Z1_1 | CRPC- | Oct. 19, 2016 | Tissue | 0.9 | 2.87 | 0.643923 | 207 | 125 | Bone | NA |
| | Adeno | | | | | | | | | |
| WCM63_Z19_1 | CRPC- | Feb. 27, 2017 | Tissue | 0.01 | 2 | 0.19152 | 128 | 65 | Liver | NA |
| | Adeno | | | | | | | | | |
| WCM63_Z19_2 | CRPC- | Feb. 27, 2017 | Tissue | 0.65 | 3.04 | 0.838218 | 242 | 137 | Liver | NA |
| | Adeno | | | | | | | | | |
| WCM63_Z2_1 | CRPC- | Aug. 26, 2013 | Tissue | 0.94 | 1.88 | 0.545437 | 171 | 111 | Prostate | NA |
| | Adeno | | | | | | | | | |
| WCM677_Z12_1 | CRPC- | Apr. 13, 2016 | Tissue | 0.99 | 1.94 | 0.318251 | 178 | 89 | Lymphnode | NA |
| | NE | | | | | | | | | |
| WCM677_Z16_1 | CRPC- | Apr. 13, 2016 | Tissue | 0.97 | 1.94 | 0.323656 | 195 | 100 | Lung | NA |
| | NE | | | | | | | | | |
| WCM677_Z3_1 | CRPC- | Nov. 13, 2015 | Tissue | 0.97 | 1.96 | 0.323568 | 214 | 117 | Bone | NA |
| | NE | | | | | | | | | |
| WCM677_Z6_1 | CRPC- | Apr. 13, 2016 | Tissue | 0.99 | 2.01 | 0.338037 | 187 | 107 | Peritoneum | NA |
| | NE | | | | | | | | | |
| WCM677_Z7_1 | CRPC- | Apr. 13, 2016 | Tissue | 0.99 | 2.03 | 0.362 | 202 | 111 | Bone | NA |
| | NE | | | | | | | | | |
| WCM677_Z9_1 | CRPC- | Apr. 13, 2016 | Tissue | 0.99 | 2.01 | 0.352733 | 204 | 112 | Liver | NA |
| | NE | | | | | | | | | |
| WCM6_Z5_1 | CRPC- | Aug. 22, 2013 | Tissue | 0.9 | 3.6 | 0.825182 | 271 | 158 | Lymphnode | NA |
| | Adeno | | | | | | | | | |
| WCM73_X1_1 | HNPC | Aug. 28, 2013 | Tissue | 0.35 | 1.99 | 0.077676 | 147 | 82 | Prostate | NA |
| WCM78_Z1_1 | CRPC- | Sep. 10, 2013 | Tissue | 0.65 | 2.18 | 0.243562 | 192 | 117 | Lymphnode | NA |
| | Adeno | | | | | | | | | |
| WCM7_Tissue | CRPC- | Mar. 28, 2013 | Tissue | 0.78 | 1.96 | 0.378935 | 223 | 91 | Liver | NA |
| | Adeno | | | | | | | | | |
| WCM80_Z1_1 | HNPC | Sep. 17, 2013 | Tissue | 0.69 | 1.98 | 0.096276 | 168 | 109 | Bone | NA |
| WCM89_X1_1 | CRPC- | May 22, 2015 | Tissue | 0.67 | 1.83 | 0.444335 | 164 | 92 | Lymphnode | NA |
| | Adeno | | | | | | | | | |
| WCM89_Z1_1 | CRPC- | Apr. 16, 2013 | Tissue | 0.39 | 2.04 | 0.245254 | 186 | 114 | Lymphnode | NA |
| | Adeno | | | | | | | | | |
| WCM89_Z12_1 | CRPC- | Mar. 22, 2010 | Tissue | 0.41 | 2.09 | 0.007271 | 299 | 174 | Prostate | NA |
| | Adeno | | | | | | | | | |
| WCM89_Z17_1 | CRPC- | Mar. 22, 2010 | Tissue | 0.4 | 2.1 | 0.163111 | 441 | 274 | Prostate | NA |
| | Adeno | | | | | | | | | |
| WCM89_ZLN1_1 | CRPC- | Mar. 22, 2010 | Tissue | 0.38 | 2.11 | 0.100778 | 400 | 247 | Lymphnode | NA |
| | Adeno | | | | | | | | | |
| WCM90_Z1_1 | CRPC- | Oct. 8, 2013 | Tissue | 0.7 | 3.1 | 0.898564 | 173 | 100 | Liver | NA |
| | Adeno | | | | | | | | | |
| WCM90_Z19_1 | CRPC- | Oct. 8, 2013 | Tissue | 0.3 | 2.01 | 0.380803 | 130 | 87 | Lymphnode | NA |
| | Adeno | | | | | | | | | |
| WCM90_Z21_1 | CRPC- | Oct. 8, 2013 | Tissue | 0.83 | 2 | 0.507591 | 178 | 110 | Liver | NA |
| | Adeno | | | | | | | | | |
| WCM90_Z25_1 | CRPC- | Oct. 8, 2013 | Tissue | NA | 1.97 | NA | 141 | 96 | Lymphnode | NA |
| | Adeno | | | | | | | | | |
| WCM90_Z29_1 | CRPC- | Oct. 8, 2013 | Tissue | 0.25 | 2.43 | 0.800655 | 154 | 95 | Bone | NA |
| | Adeno | | | | | | | | | |
| WCM91_Z1_1 | CRPC- | Aug. 18, 2014 | Tissue | 0.75 | 3 | 0.840813 | 138 | 89 | Prostate | NA |
| | Adeno | | | | | | | | | |
| WCM9_X1_1 | CRPC- | Jan. 30, 2014 | Tissue | 0.23 | 2.4 | 0.868995 | 222 | 89 | Bone | NA |
| | Adeno | | | | | | | | | |
| WCM9_Z3-1 | CRPC- | Apr. 17, 2013 | Tissue | 0.5 | 2.15 | 0.249972 | 157 | 73 | Prostate | NA |
| | Adeno | | | | | | | | | |

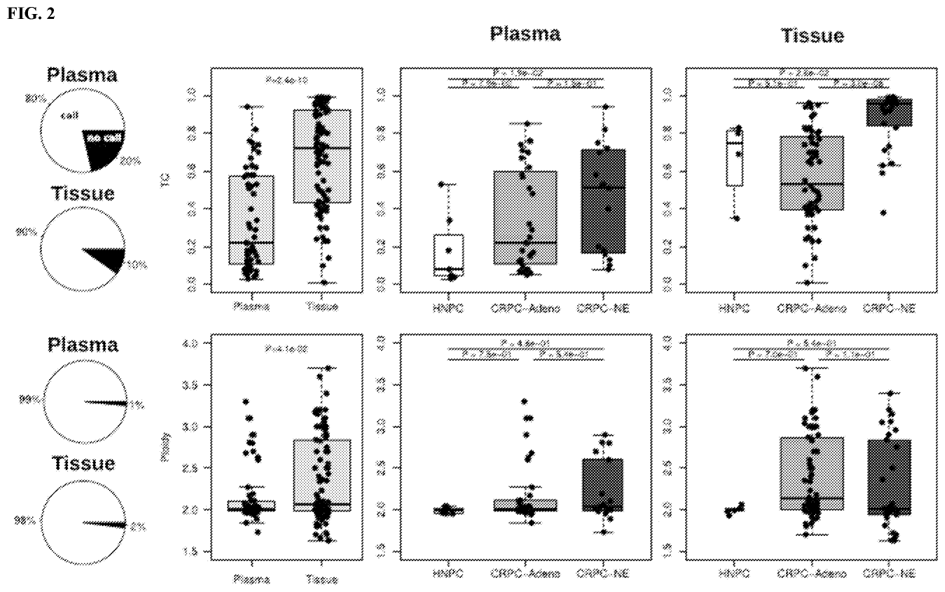

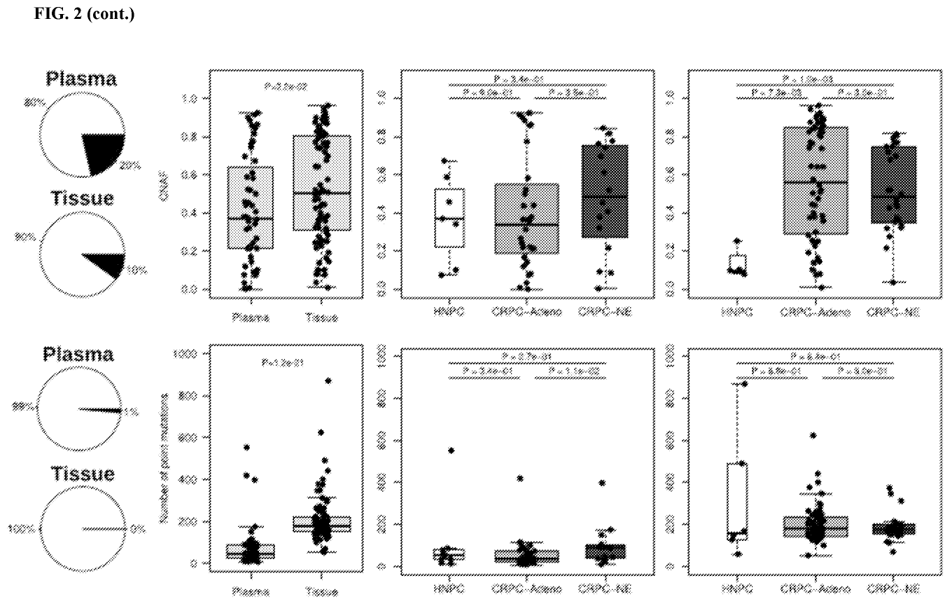

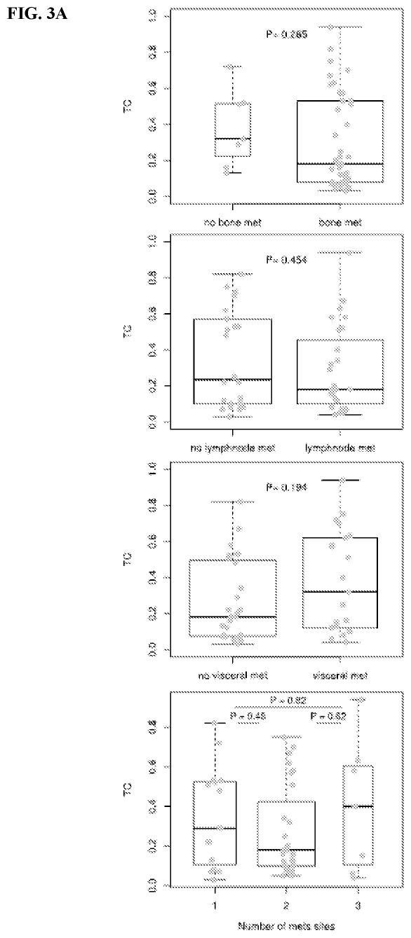

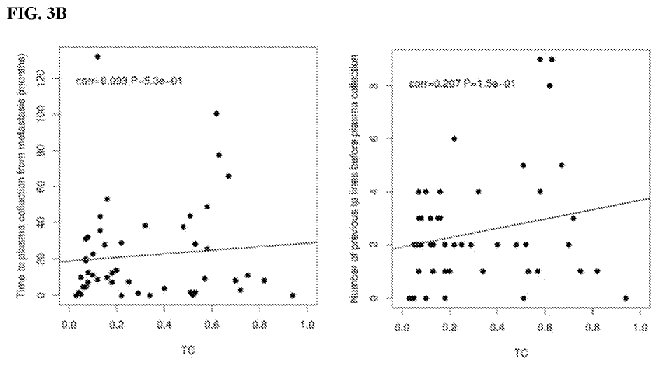

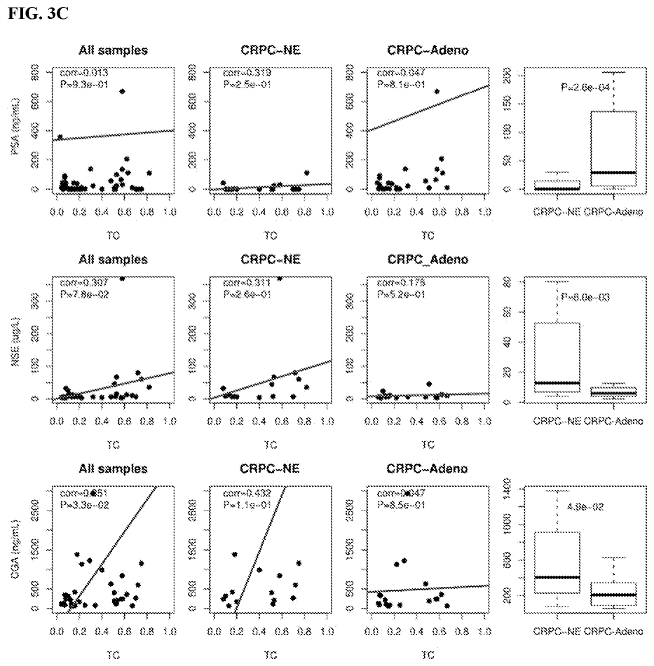

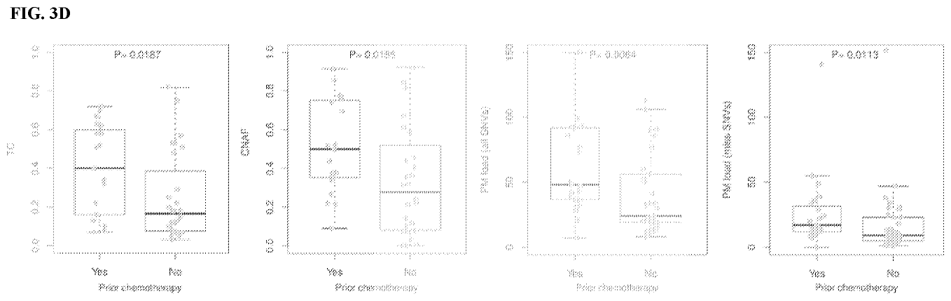

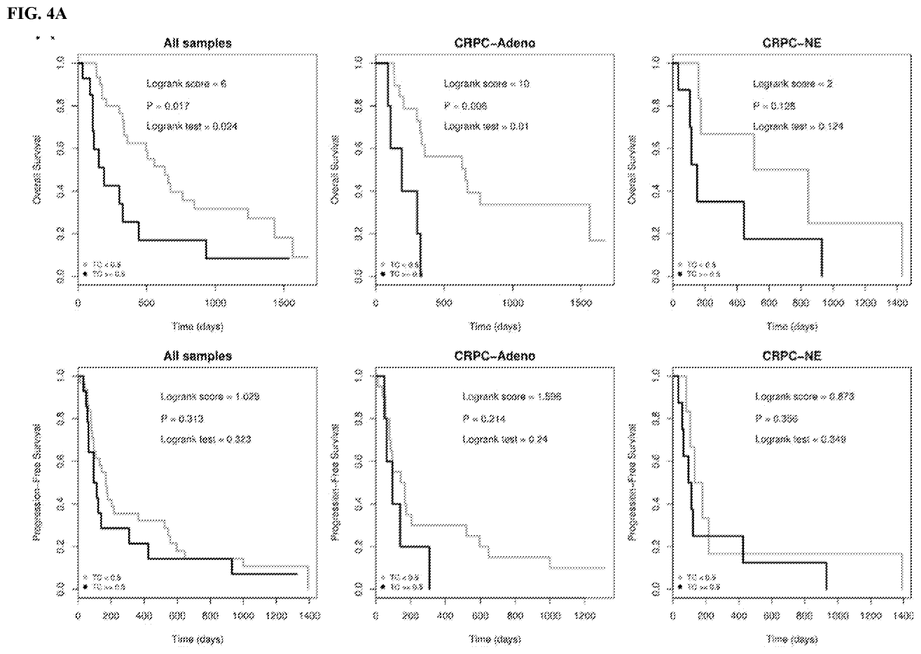

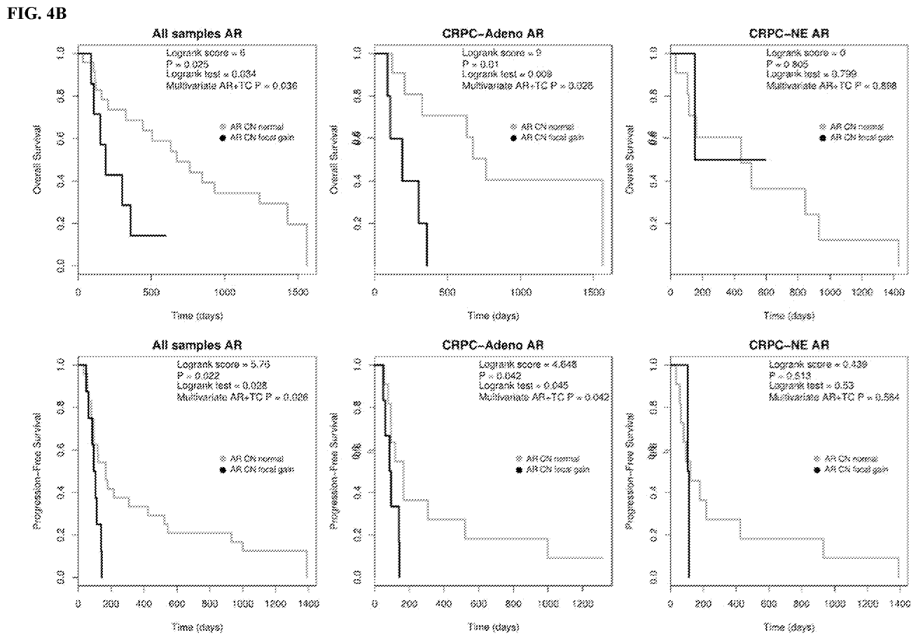

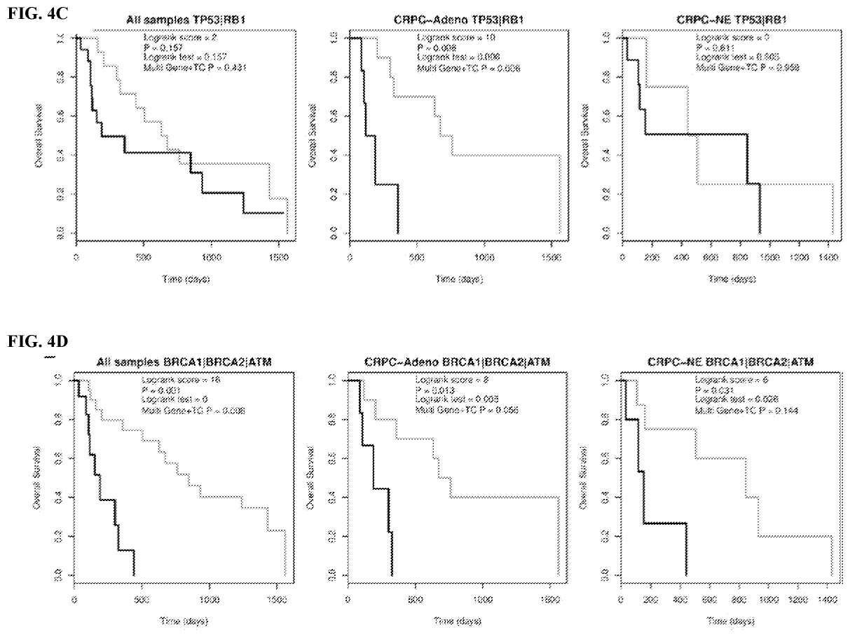

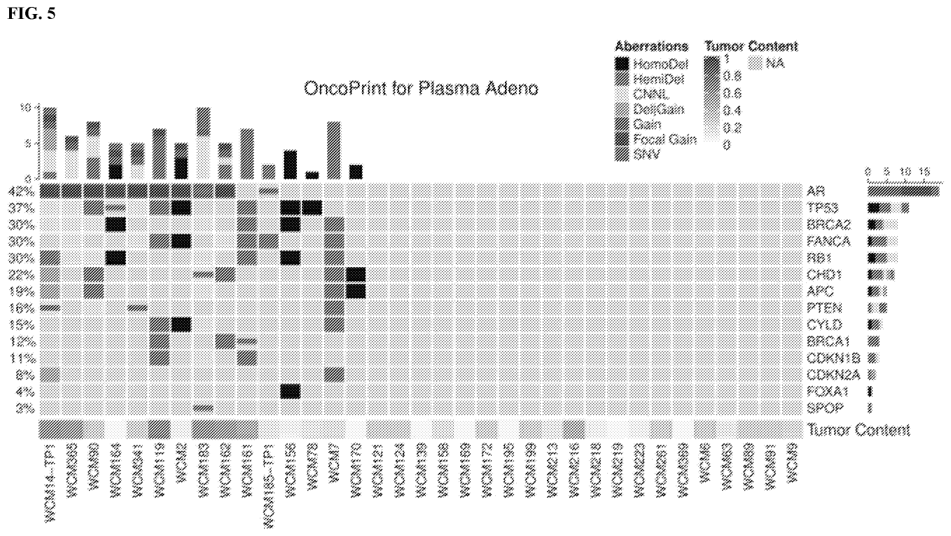

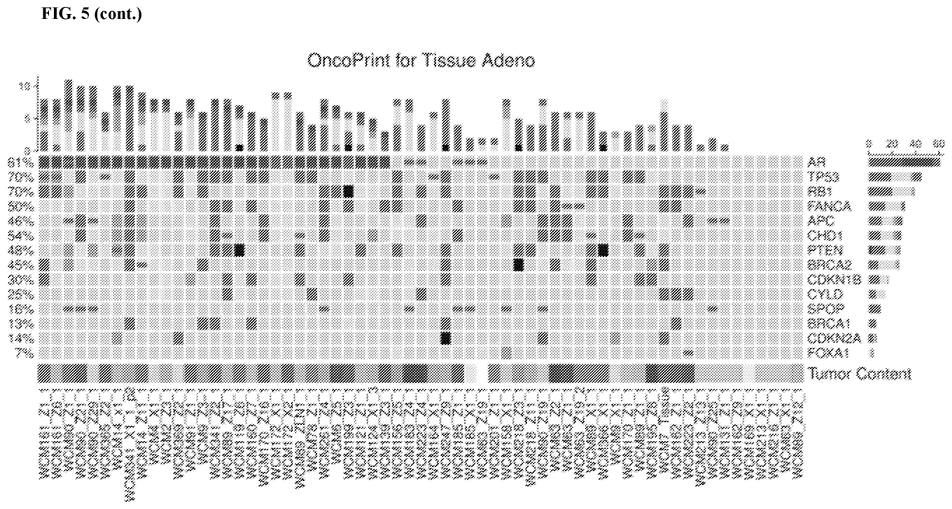

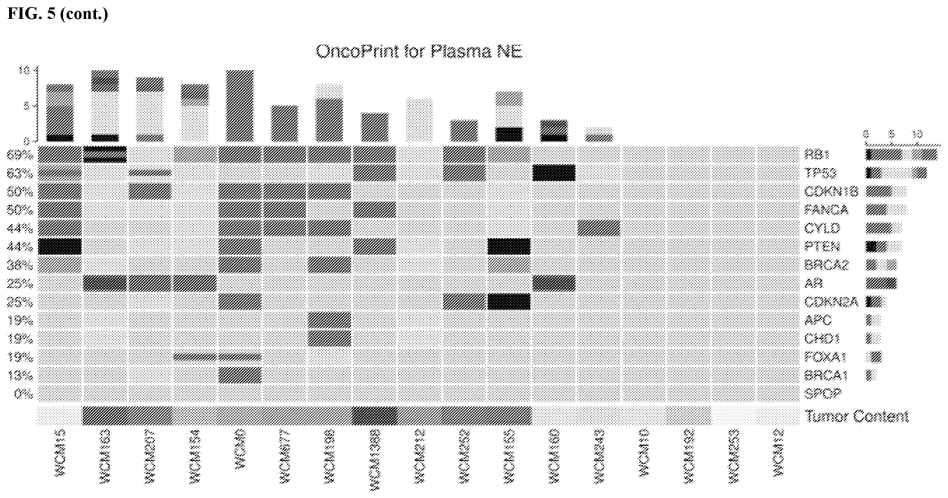

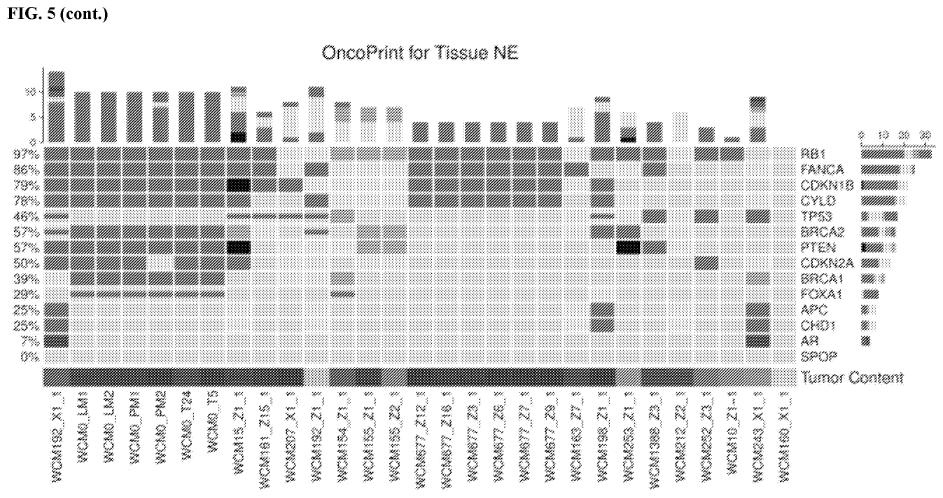

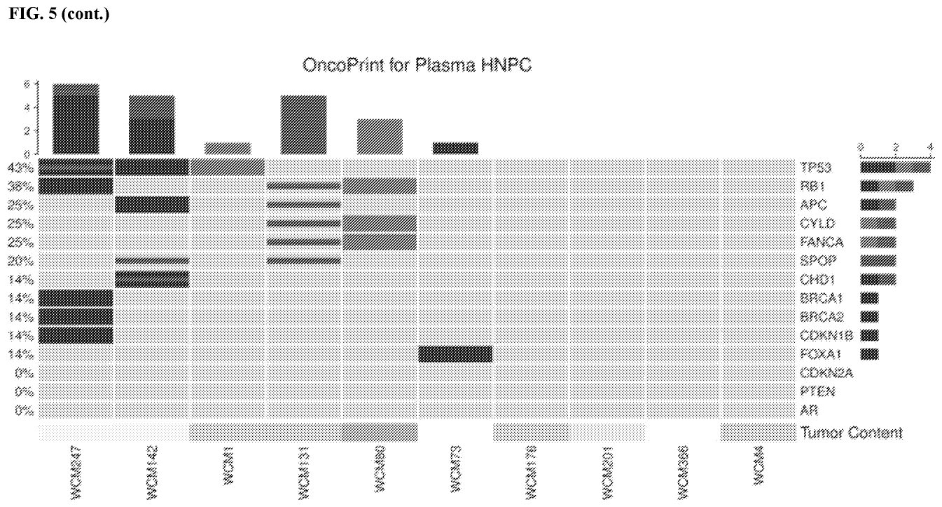

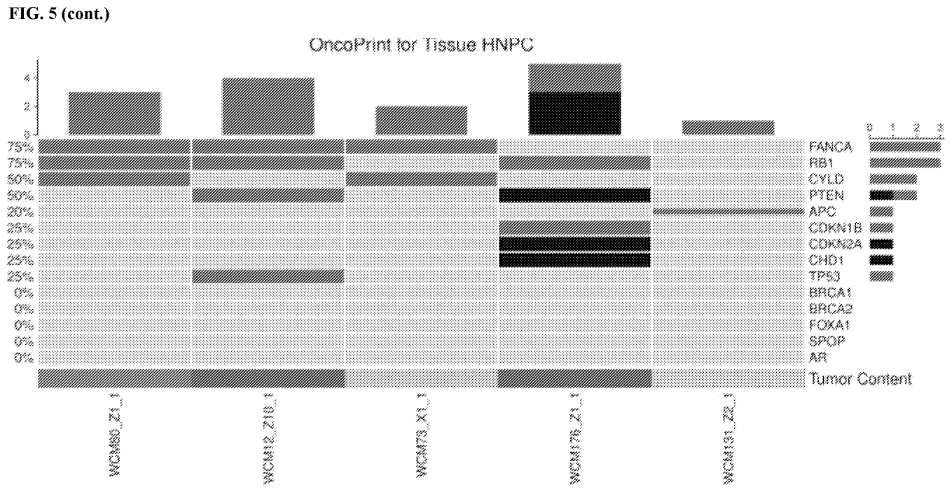

Tumor/normal whole exome sequencing (WES) was performed on cell free DNA (cfDNA) extracted from plasma and germline DNA from peripheral blood mononuclear cells (PBMCs) with a median depth of coverage of 357× for plasma samples and 105× for germline samples. Median cfDNA tumor content (TC) was 22% [3%-94%], and TC did not associate with histology subtype (i.e., adenocarcinoma or CRPC-NE) (FIG. 2), number or type of metastatic sites, prostate specific antigen (PSA) levels, serum neuroendocrine markers, or number of prior systemic therapies (FIGS. 3A-3C). Prior exposure to cytotoxic chemotherapy was associated with higher cfDNA TC (FIG. 3D), and no statistical difference in TC was observed with respect to presence or absence of sites of metastasis (bone, lymph node or visceral) in chemotherapy-treated patients. In patients with CRPC-Adeno, a cfDNA TC >50% was associated with inferior prognosis (p=0.01, FIG. 4A), but this was not observed in the CRPC-NE subclass. Tumor mutation load and copy number burden in ctDNA did not differ significantly between tumor histologies, but both were higher in patients that received prior chemotherapy compared to those that did not (FIG. 3D). Recurrent genomic alterations observed in plasma and in tissue samples of study cohort samples detected by WES are shown in FIGS. 1B-1C and FIG. 5.

Overall, copy number alterations and point mutations involving commonly altered prostate cancer associated genes (Table 12) detected in metastatic biopsy tissue samples were evident in patient-matched ctDNA (median concordance 71%, range from 0.29 to 1). This degree of concordance was similar to other targeted DNA and WES prostate cancer ctDNA studies (Wyatt et al. (2017) J Natl Cancer Inst 109: doi: 10.1093/jnci/djx118; Adalsteinsson et al. (2017) Nat Commun 8:1324). As expected based on prior studies (Abida et al. (2019) Proc Natl Acad Sci USA 116:11428-11436; Beltran et al. (2016) Nat Med 22:298-305), alterations involving RB1, TP53, and CYLD were more common in the ctDNA and biopsy tissues of CRPC-NE patients, AR alterations more common in CRPC-Adeno. There were no significant differences in the frequency of DNA repair gene aberrations involving BRCA1, BRCA2, ATM between the subtypes (FIGS. 1B and 5, Table 8). SPOP mutations were notably absent in CRPC-NE. The most common genomic alterations in ctDNA of mPCA patients included TP53 (43%), RB1 (38%), FANCA (25%), in CRPC-Adeno were AR (42%), TP53 (37%), and in CRPC-NE were RB1 (69%), TP53 (63%). As observed in prior studies (Romanel et al. (2015) Sci Transl Med 7:312re310; Azad et al. (2015) Clin Cancer Res 21:2315-2324), the presence of AR aberrations in the ctDNA of CRPC-Adeno patients was associated with inferior overall survival (p=0.009), which was maintained after adjustment for TC (multivariate p=0.028, FIG. 4B). Of note, AR focal gains could be either persistent or dynamic when comparing serial samples in individuals with CRPC-Adeno (FIG. 1D) supporting evolution of somatic AR aberrations. The prognostic value of other specific gene aberrations in CRPC-Adeno was consistent with a recent study by Annala et al (Annala et al. (2018) Cancer Discov 8:444-457). The presence of TP53 and/or RB1 loss of function alterations in the ctDNA of patients with CRPC-Adeno (but not CRPC-NE) associated with worse overall survival (p=0.006), including after accounting for TC (multivariate p=0.006, FIG. 4C). Somatic alterations involving BRCA1, BRCA2, or ATM were also prognostic across the cohort (p=0.008 multivariate, FIG. 4D).

TABLE 8
Frequency of somatic aberrations in CRPC-NE vs CRPC-Adeno

| | | | | | | | | |
|---|---|---|---|---|---|---|---|---|
| | | Tot | Freq. | | Tot | Freq. in | | |
| Gene | CRPC- | CRPC- | CRPC- | CRPC- | CRPC- | CRPC- | P-value | P-value |
| Symbol | NE | NE | NE | Adeno | Adeno | Adeno | (two sided) | (one sided) |

SCNA (gain for AR and allele-specific CN = 0|0, 1|0, 2|0 or N|0 for all other genes)

| | | | | | | | | |
|---|---|---|---|---|---|---|---|---|
| BRCA1 | 2 | 16 | 0.125 | 2 | 26 | 0.077 | 0.628339141 | 0.494773519 |
| BRCA2 | 6 | 16 | 0.375 | 8 | 27 | 0.296 | 0.738806798 | 0.418793442 |
| CDKN2A | 4 | 15 | 0.267 | 2 | 26 | 0.077 | 0.168343568 | 0.117140247 |
| APC | 3 | 16 | 0.188 | 5 | 27 | 0.185 | 1 | 0.641746861 |
| CHD1 | 3 | 16 | 0.188 | 6 | 27 | 0.222 | 1 | 0.739730625 |
| RB1 | 11 | 16 | 0.688 | 8 | 27 | 0.296 | 0.024835363 | 0.014353073 |
| CDKN1B | 8 | 16 | 0.500 | 3 | 27 | 0.111 | 0.009547745 | 0.007281075 |
| TP53 | 10 | 16 | 0.625 | 9 | 27 | 0.333 | 0.111247733 | 0.061240502 |
| CYLD | 7 | 16 | 0.438 | 4 | 27 | 0.148 | 0.067919147 | 0.042185777 |
| PTEN | 7 | 15 | 0.467 | 3 | 25 | 0.120 | 0.023745223 | 0.019889009 |
| FANCA | 8 | 16 | 0.500 | 8 | 27 | 0.296 | 0.208984468 | 0.156451431 |
| AR | 2 | 16 | 0.125 | 8 | 23 | 0.348 | 0.1523318 | 0.1149152 |

Non-synonymous SNV

| | | | | | | | | |
|---|---|---|---|---|---|---|---|---|
| TP53 | 3 | 16 | 0.1875 | 2 | 35 | 0.057 | 0.3445601 | 0.17228 |
| FOXA1 | 2 | 16 | 0.125 | 1 | 35 | 0.029 | 0.4735416 | 0.2367708 |
| SPOP | 0 | 16 | 0 | 3 | 35 | 0.086 | 1 | 1 |
| RB1 | 2 | 16 | 0.125 | 0 | 35 | 0.000 | 0.174922 | 0.08746101 |
| BRCA2 | 0 | 16 | 0 | 0 | 35 | 0.000 | 1 | 1 |

Non-synonymous SNVs or SCNA (gain for AR and allele-specific CN = 0|0, 1|0, 2|0 or N|0 for all
other genes)

| | | | | | | | | |
|---|---|---|---|---|---|---|---|---|
| RB1 | 11 | 16 | 0.688 | 8 | 26 | 0.308 | 0.025814876 | 0.018260359 |
| AR | 2 | 16 | 0.125 | 9 | 24 | 0.375 | 0.1478246 | 0.08252297 |

Bold indicates p-values < 0.05

TABLE 12
Concordance in genomic losses across plasma
and matched tissue samples (TC > 0.1)

| Hugo | Fraction of loss signal in | Fraction of loss signal in |
|---|---|---|
| symbol | tissue found in plasma | plasma found in tissue |
| TP53 | 0.89 | 0.67 |
| RB1 | 0.76 | 0.54 |
| FANCA | 0.94 | 0.74 |
| BRCA2 | 0.71 | 0.71 |
| CDKN1B | 0.62 | 0.38 |
| CYLD | 0.69 | 0.64 |
| PTEN | 1 | 0.43 |
| CHD1 | 0.55 | 0.55 |
| APC | 0.5 | 0.5 |
| CDKN2A | 0.75 | 0.38 |
| BRCA1 | 0.29 | 0.33 |

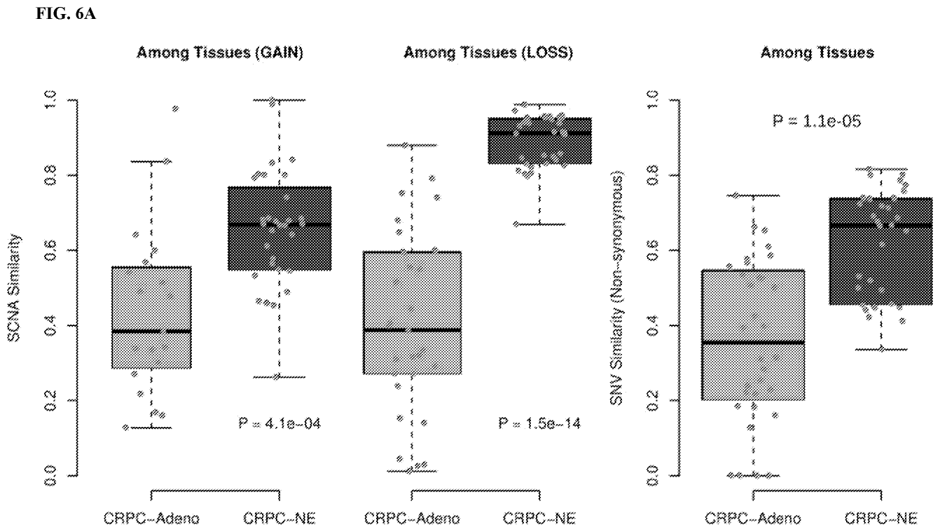

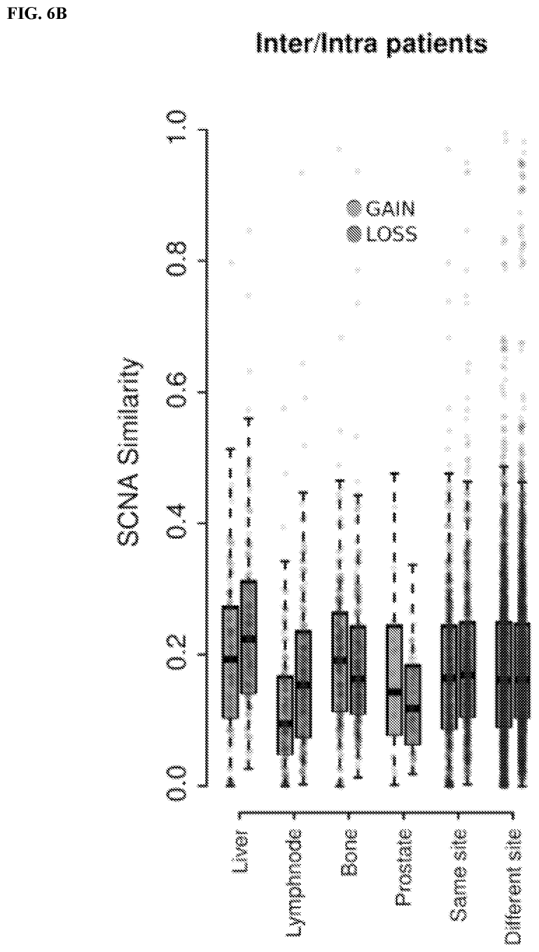

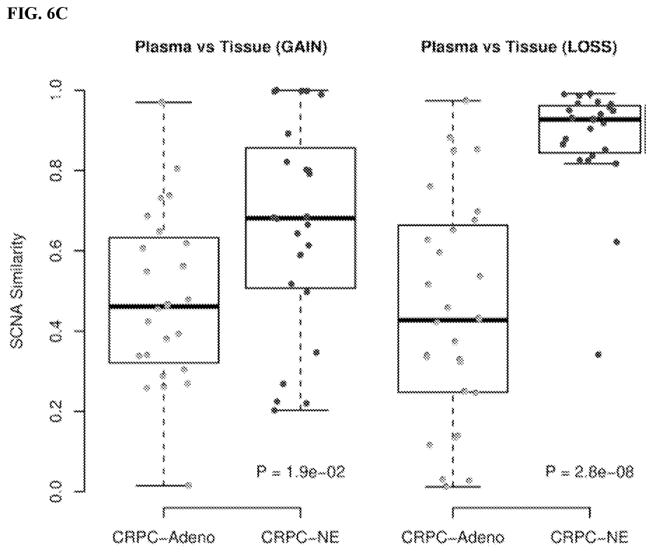

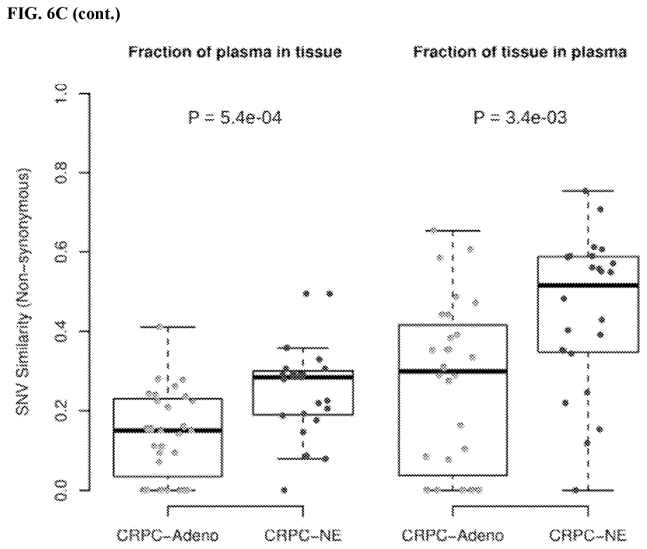

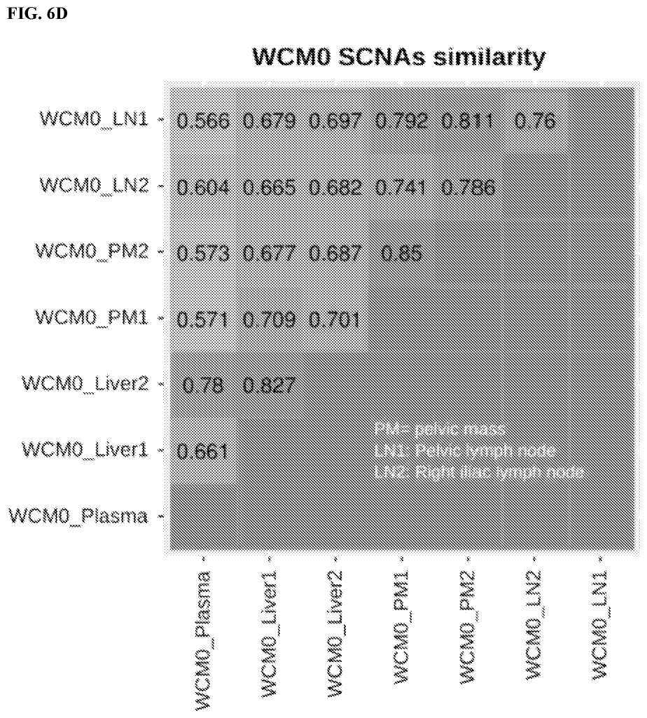

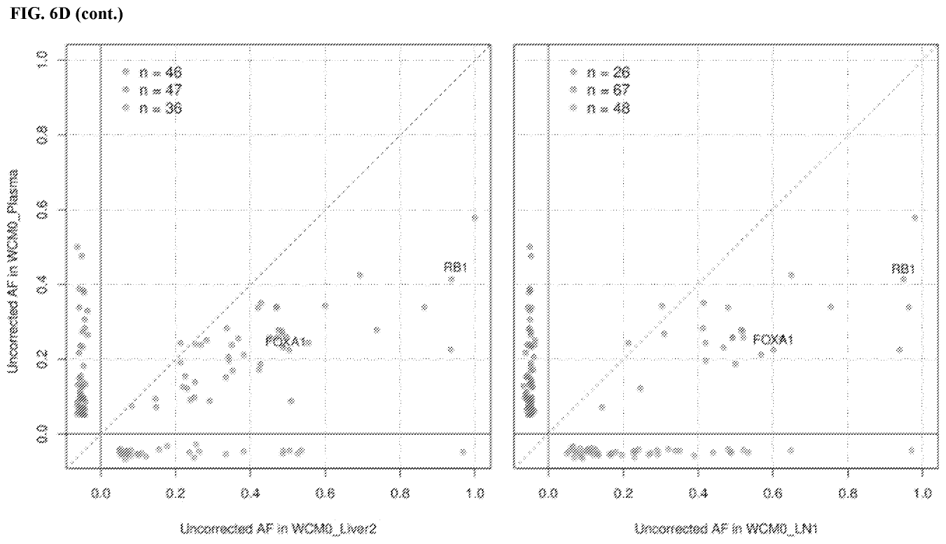

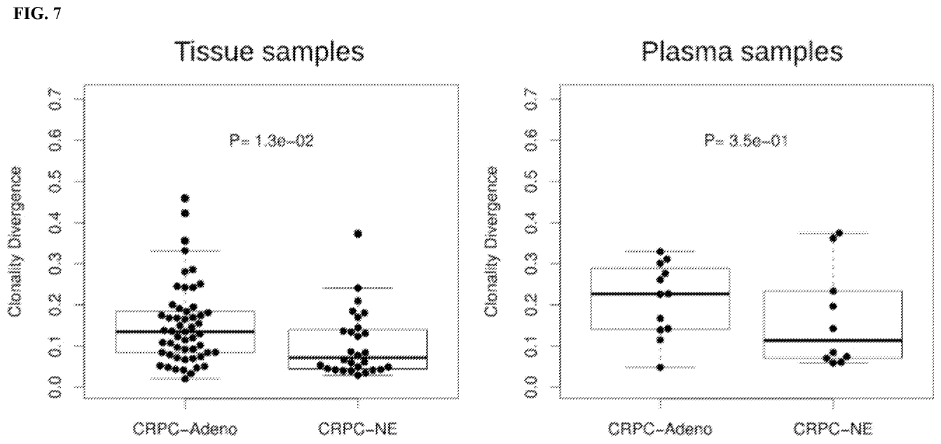

A subset of alterations detected in ctDNA was not detected in matched tissue samples (FIG. 5); though false negative calls cannot be excluded, it is likely that intra-patient tumor heterogeneity is a prominent reason for these differences, whereby the sampled metastasis did not fully capture the genomic status of the patient's cancer burden. In order to quantify and contextualize the degree of CRPC-NE intra- and inter-patient heterogeneity by comparing metastatic tissue and liquid biopsies, the distributions of genome-wide heterogeneity levels among all metastatic tissue biopsies of both CRPC-Adeno and CRPC-NE were first built using their genomic profiles (WES data). While there was generally genomic concordance across metastatic tissues in either subtype (FIG. 6A), the CRPC-NE subtype demonstrated significantly greater inter-individual similarity both for somatic copy number alterations (SCNA) and for single nucleotide variants (SNVs) (FIG. 6A). Importantly, this finding was irrespective of the anatomic site of the metastatic biopsy assessed as indicated by similarities stratified by site and across sites (FIG. 6B). Higher intra-patient genomic similarity was also observed when comparing ctDNA and biopsy samples within individuals with CRPC-NE (FIGS. 6C and 7) compared to CRPC-Adeno patients. If a plasma sample represents the genomics of all or most of an individual's metastases released into the circulation, a higher concordance between ctDNA and single site biopsy indicates lower intra-patient tumor heterogeneity in CRPC-NE. However, differential contribution or release rates of individual metastases into the circulation cannot be fully excluded. For instance, inspection of the relative contributions into the plasma of six spatially distinct metastases (with 74% of average genomic similarity with one another) of one CRPC-NE patient all obtained at the same time-point supported a higher contribution of liver metastasis tumor alterations in cfDNA compared with other sites (FIG. 6D).

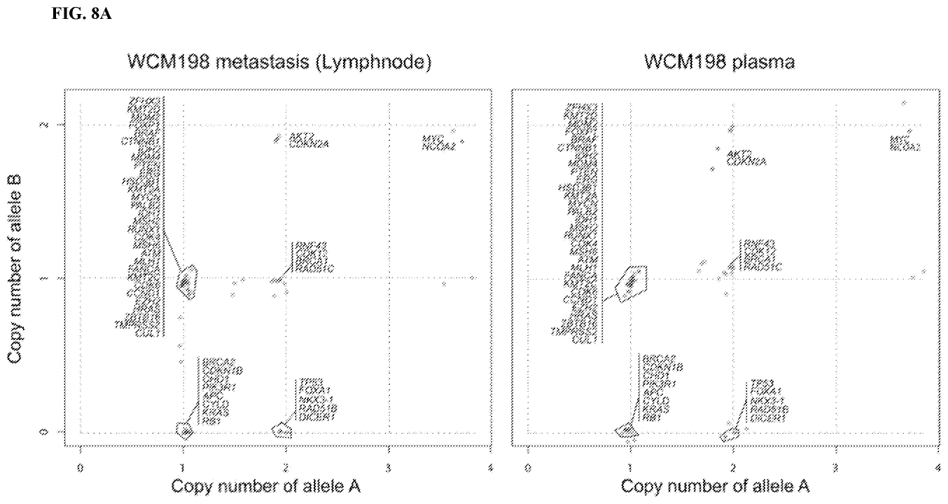

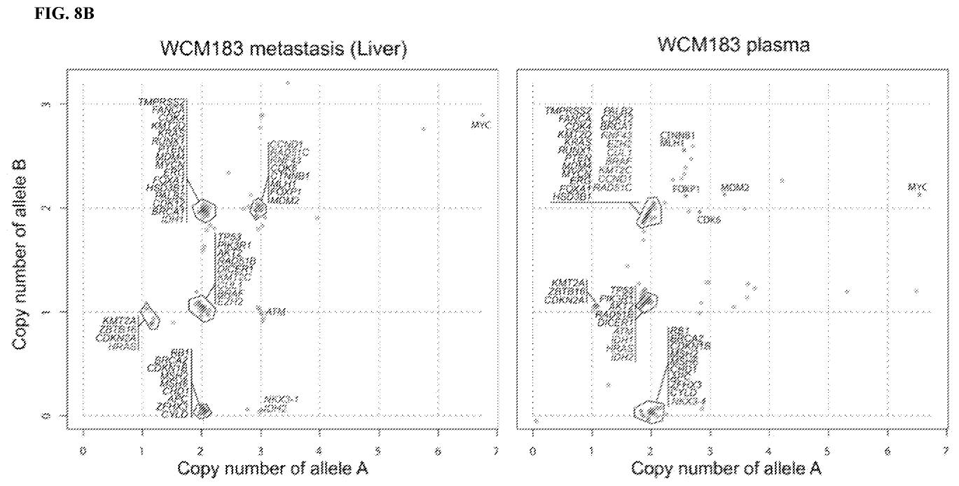

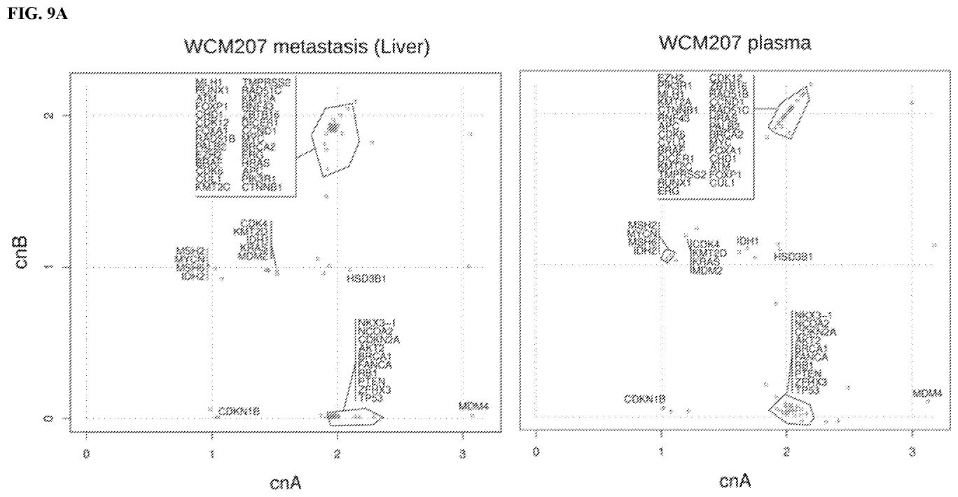

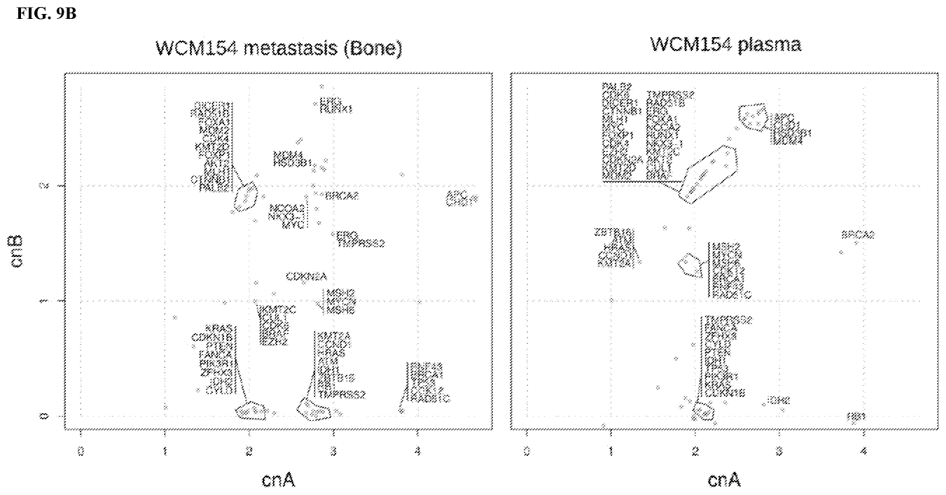

In order to further explore intra-patient heterogeneity across the cohort, allele-specific copy number analysis (Prandi and Demichelis (2019) Curr Protoc Bioinformatics 67:e81) of WES data was leveraged. While the vast majority of alterations were identified as shared between ctDNA and metastatic biopsies (FIGS. 8A and 9A), this higher resolution analysis allowed for the identification of distinct subclonal differences (as illustrated by cases examples in FIGS. 8B and 9B; blue genes demonstrate different allele-specific status between tissue and plasma samples). These differences can represent private events in metastatic lesions that are therefore differentially represented in the circulation; diversity can be restricted to a small set of lesions (FIG. 8B) or to a wide range of structural differences, resulting in allele-specific diversity (FIG. 9B). Overall these data support subclonal heterogeneity in metastatic lesions that can not be detected by single site biopsy but can also contribute to the development of therapy resistance.

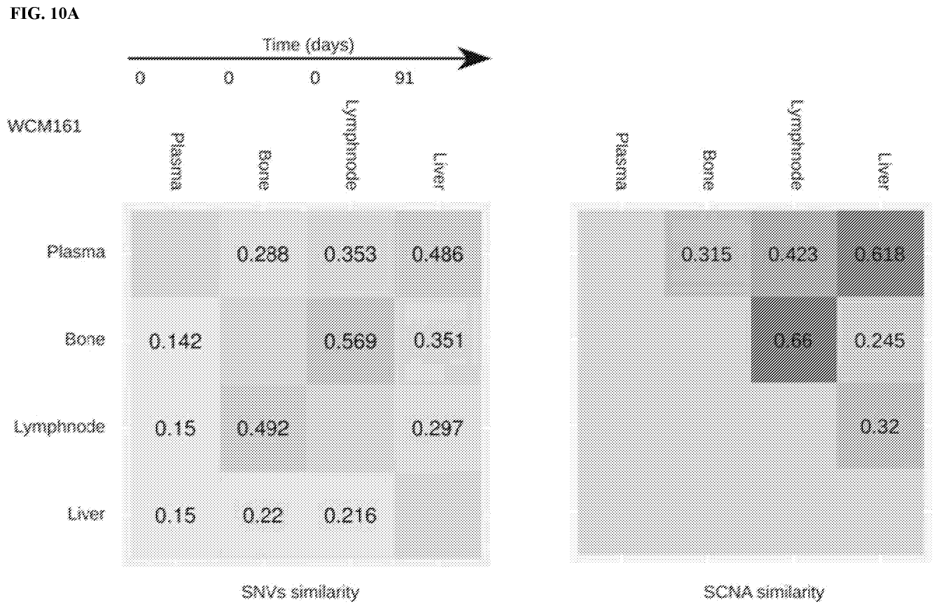

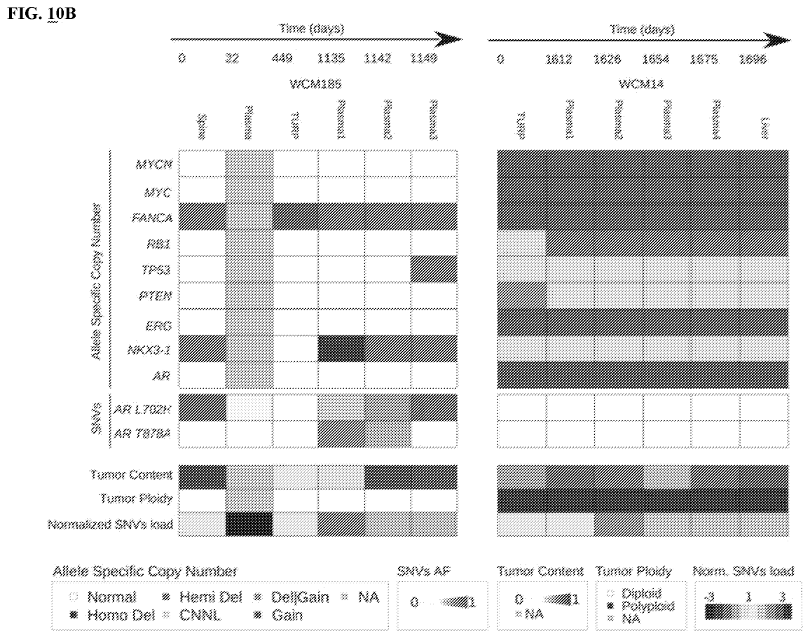

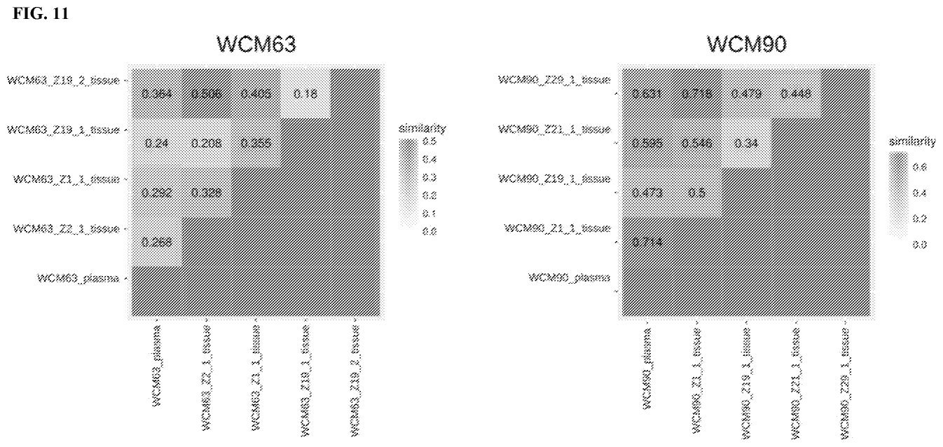

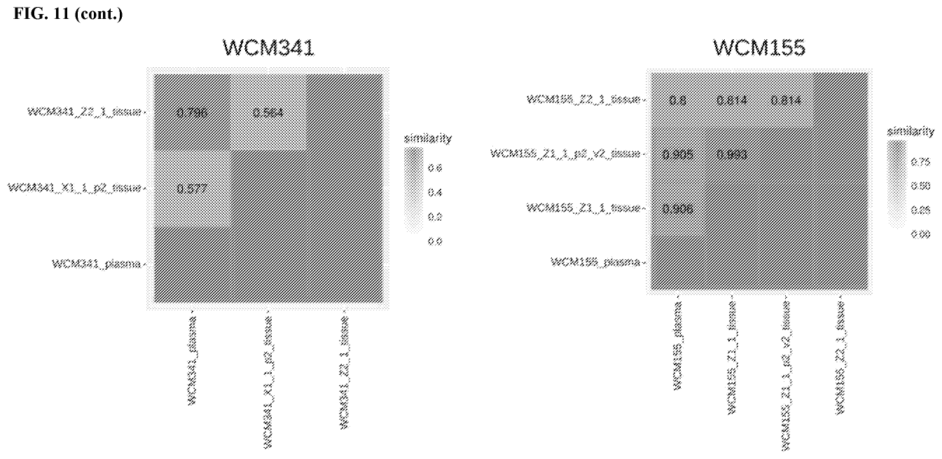

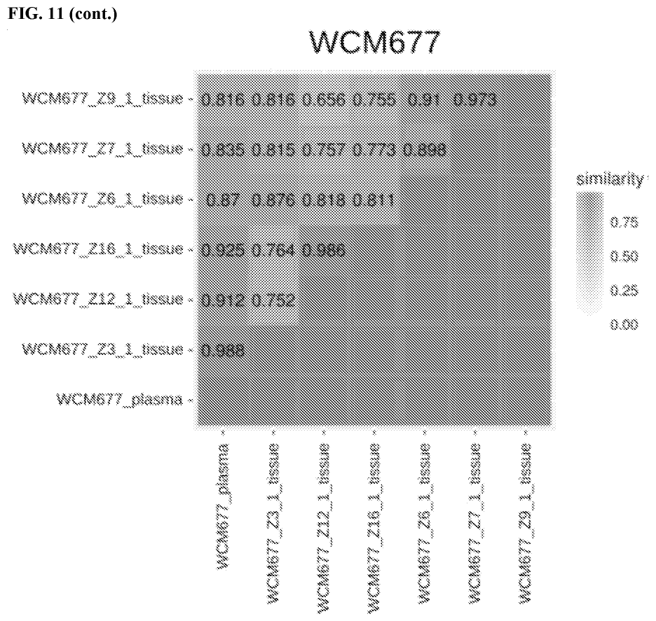

To explore how genomic alterations can change with time, serial tumor and blood samples from individuals were evaluated (FIGS. 10 and 11, Table 13). In patient WCM161, metastatic biopsy time-points during clinical progression from CRPC-Adeno to CRPC-NE were tracked. Interestingly, the plasma sample obtained at the time of CRPC-Adeno with lymph node and bone metastases present displayed a genomic ctDNA profile most similar (based on whole exome-wide comparison) to the CRPC-NE liver metastasis observed on imaging and biopsied three months later at the time of progression on abiraterone (FIG. 10A), supporting the presence of detectable resistant clones in the circulation prior to the development of clinical features of CRPC-NE or liver metastases.

TABLE 13
Somatic copy number aberration-based distance
among plasma and matched tissue samples

| Plasma | Tissue | Similarity LOSS | Similarity GAIN | Type |
|---|---|---|---|---|
| WCM156 | WCM156_Z5_1 | 0.46 | 0.33 | CRPC-Adeno |
| WCM2 | WCM2_Z3_1 | 0.26 | 0.32 | CRPC-Adeno |
| WCM15 | WCM15_Z1_1 | 0.68 | 0.9 | CRPC-NE |
| WCM218 | WCM218_Z11_1 | NA | 0.03 | CRPC-Adeno |
| WCM218 | WCM218_Z3_1 | NA | 0.03 | CRPC-Adeno |
| WCM63 | WCM63_Z2_1 | 0.29 | 0.25 | CRPC-Adeno |
| WCM63 | WCM63_Z1_1 | 0.34 | 0.25 | CRPC-Adeno |
| WCM63 | WCM63_Z19_2 | 0.39 | 0.34 | CRPC-Adeno |
| WCM160 | WCM160_Z7_1 | 0.56 | 0.52 | CRPC-Adeno |
| WCM185 | WCM185_X1_1 | NA | 0.13 | CRPC-Adeno |
| WCM185 | WCM185_Z1_1 | 0.38 | 0.65 | CRPC-Adeno |
| WCM78 | WCM78_Z1_1 | 0.3 | 0.14 | CRPC-Adeno |
| WCM243 | WCM243_X1_1 | 0.8 | 0.84 | CRPC-NE |
| WCM158 | WCM158_X1_1 | 0.01 | 0.01 | CRPC-Adeno |
| WCM7 | WCM7_Tissue | 0.97 | 0.88 | CRPC-Adeno |
| WCM9 | WCM9_Z3-1 | NA | 0.12 | CRPC-Adeno |
| WCM341 | WCM341_X1_1_p2 | 0.48 | 0.68 | CRPC-Adeno |
| WCM341 | WCM341_Z2_1 | 0.62 | 0.97 | CRPC-Adeno |
| WCM90 | WCM90_Z1_1 | 0.73 | 0.7 | CRPC-Adeno |
| WCM90 | WCM90_Z19_1 | 0.61 | 0.34 | CRPC-Adeno |
| WCM90 | WCM90_Z21_1 | 0.34 | 0.85 | CRPC-Adeno |
| WCM90 | WCM90_Z29_1 | 0.8 | 0.46 | CRPC-Adeno |
| WCM154 | WCM154_Z1_1 | 0.8 | 0.82 | CRPC-NE |
| WCM0 | WCM0_LM1 | 0.5 | 0.82 | CRPC-NE |
| WCM0 | WCM0_LM2 | 0.59 | 0.97 | CRPC-NE |
| WCM0 | WCM0_PM1 | 0.22 | 0.92 | CRPC-NE |
| WCM0 | WCM0_PM2 | 0.22 | 0.93 | CRPC-NE |
| WCM0 | WCM0_T24 | 0.27 | 0.94 | CRPC-NE |
| WCM0 | WCM0_T5 | 0.2 | 0.93 | CRPC-NE |
| WCM161 | WCM161_Z1_1 | 0.26 | 0.37 | CRPC-Adeno |
| WCM161 | WCM161_Z15_1 | 0.61 | 0.62 | CRPC-NE |
| WCM161 | WCM161_Z6_1 | 0.42 | 0.42 | CRPC-Adeno |
| WCM198 | WCM198_Z1_1 | 0.89 | 0.88 | CRPC-NE |
| WCM677 | WCM677_Z12_1 | 1 | 0.83 | CRPC-NE |
| WCM677 | WCM677_Z16_1 | 1 | 0.85 | CRPC-NE |
| WCM677 | WCM677_Z3_1 | 0.99 | 0.99 | CRPC-NE |
| WCM677 | WCM677_Z6_1 | 0.79 | 0.95 | CRPC-NE |
| WCM677 | WCM677_Z7_1 | 0.68 | 0.99 | CRPC-NE |
| WCM677 | WCM677_Z9_1 | 0.68 | 0.95 | CRPC-NE |
| WCM162 | WCM162_Z1_1 | 0.65 | 0.54 | CRPC-Adeno |
| WCM212 | WCM212_Z2_1 | 0.52 | 0.86 | CRPC-NE |
| WCM365 | WCM365_Z2_1 | 0.47 | 0.6 | CRPC-Adeno |
| WCM183 | WCM183_Z4_1 | 0.55 | 0.76 | CRPC-Adeno |
| WCM119 | WCM119_Z6_1 | 0.69 | 0.85 | CRPC-Adeno |
| WCM207 | WCM207_X1_1 | 1 | 0.97 | CRPC-NE |
| WCM14 | WCM14_x1_1 | 0.27 | 0.43 | CRPC-Adeno |
| WCM14 | WCM14_Z11_1 | 0.74 | 0.63 | CRPC-Adeno |
| WCM252 | WCM252_Z3_1 | 1 | 0.97 | CRPC-NE |
| WCM155 | WCM155_Z1_1 | 0.82 | 0.99 | CRPC-NE |
| WCM155 | WCM155_Z2_1 | 0.64 | 0.96 | CRPC-NE |
| WCM163 | WCM163_Z7_1 | 0.35 | 0.34 | CRPC-NE |
| WCM1388 | WCM1388_Z3_1 | 0.66 | 0.93 | CRPC-NE |

To delve deeper into tumor dynamics, short-interval serial time-points of two patients with metastatic CRPC progressing after multiple lines of systemic therapy (FIG. 10B) were assessed. Patient WCM185 is a patient with a rising PSA >3000 ng/ml, bone-only metastases, a clinical picture indicative of AR-driven progression. Patient WCM14 developed new visceral metastases despite a non-rising PSA, clinical features commonly observed in CRPC-NE. Three weekly serial plasma time-points of patient WCM185 were compared with WES data from his CRPC-Adeno tumor/plasma specimens collected over the three years prior. When corrected for ploidy and TC, distinct clones were identified in ctDNA including those with and without AR mutations and TP53 deletions that changed dynamically with time, indicating multiple clones within the circulation with competing frequencies. This fast evolution in tumor clones even in the absence of therapy is consistent with clonal disequilibrium. On the other hand, four serial samples of patient WCM14, as well as his tumor tissue samples collected five years earlier when he had minimal metastatic disease burden, all showed consistent alterations including MYC gain and RB1, TP53, PTEN, and BRCA2 losses across all time-points indicating these were relatively early events. Of note, concurrent loss of RB1 and TP53, frequently associated with small cell carcinomas including CRPC-NE, was detectable before he had developed clinical features of AR independence or CRPC-NE (FIG. 10B).

These data indicate that in some cases, CRPC can be dominated by a clone that arises early where, in other cases, subclones evolve and contribute to treatment resistance. By using ctDNA to track clones with a selective advantage with those that are less fit, tumor dynamics during prostate cancer progression can be better understood. In the setting of CRPC-NE, intrinsic drug resistance is likely a major factor that contributes to the development of clonal dominance. However, other biological factors, including a proliferative advantage, the local microenvironment, and epigenetic alterations also likely play a role.

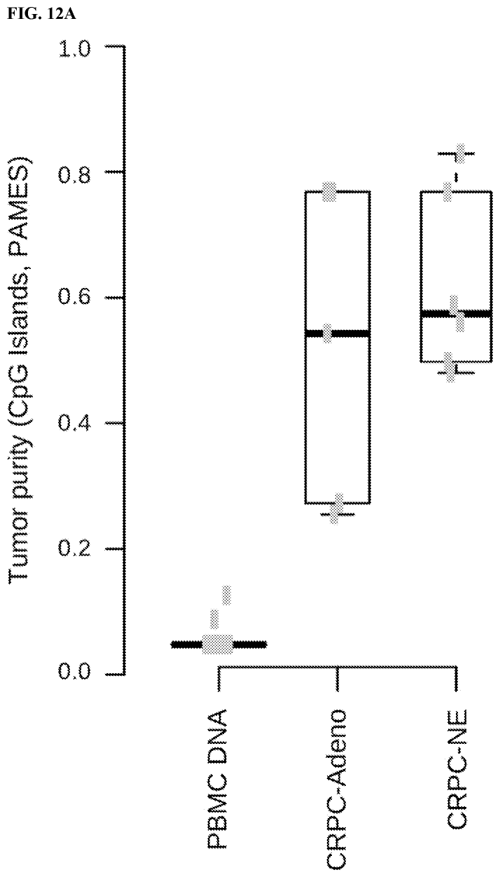

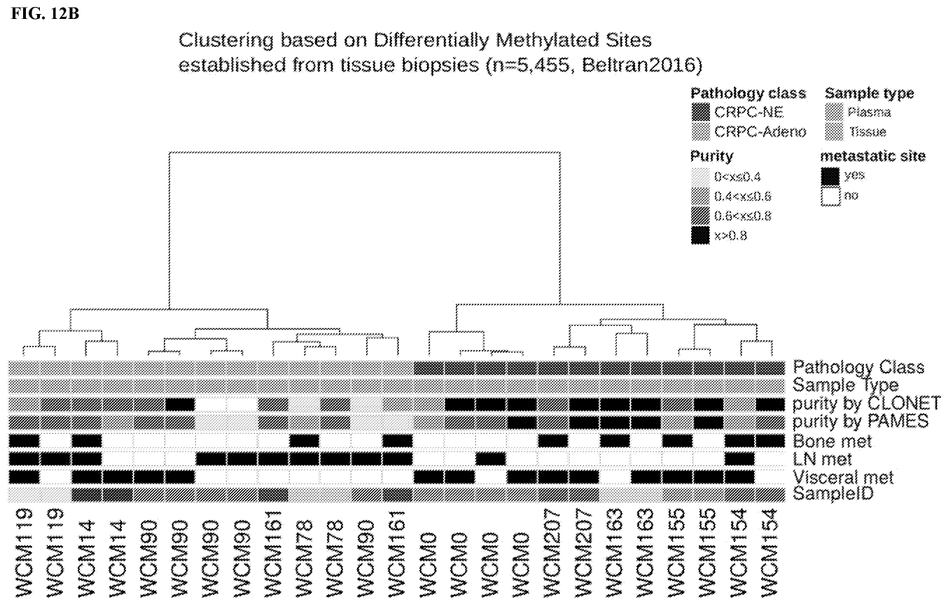

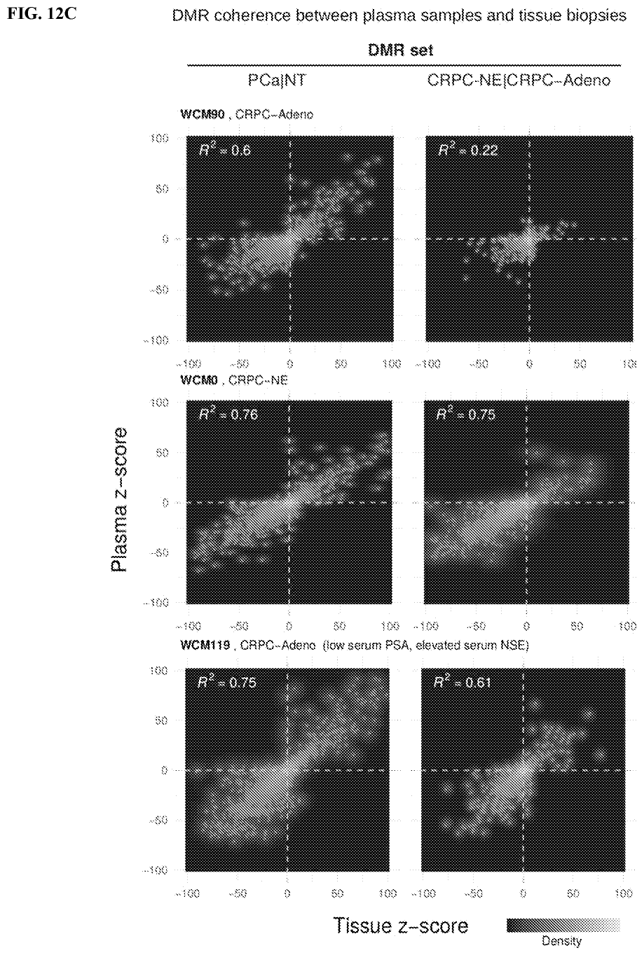

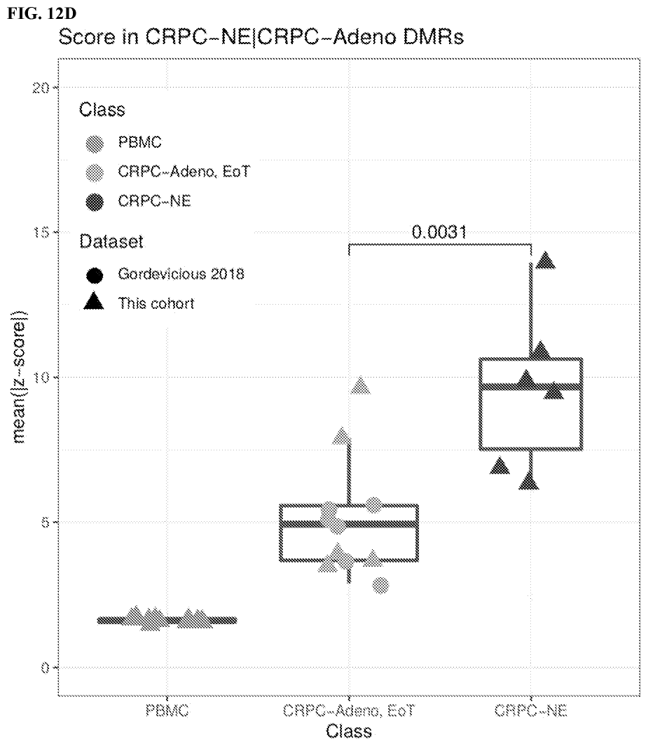

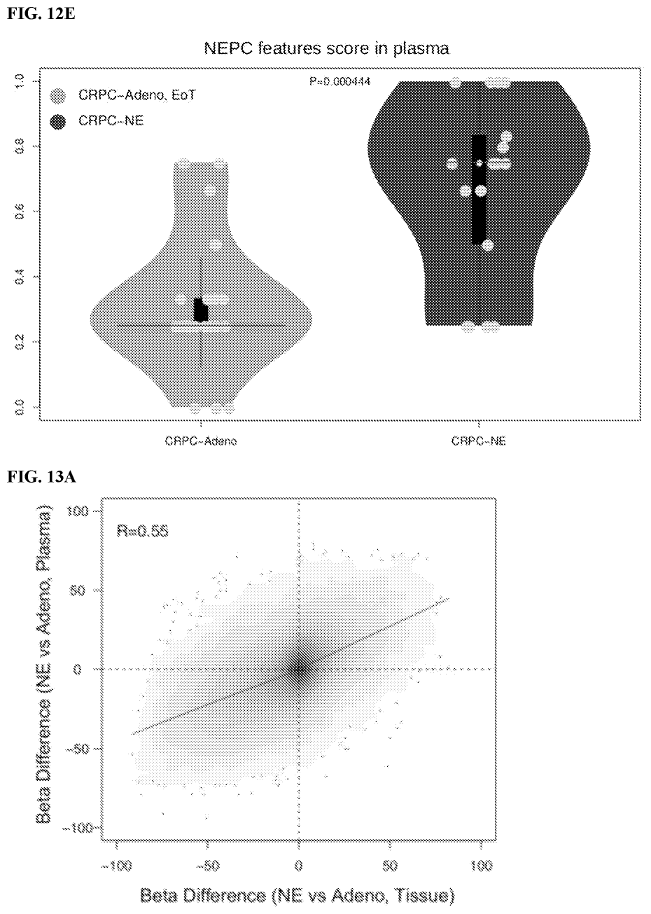

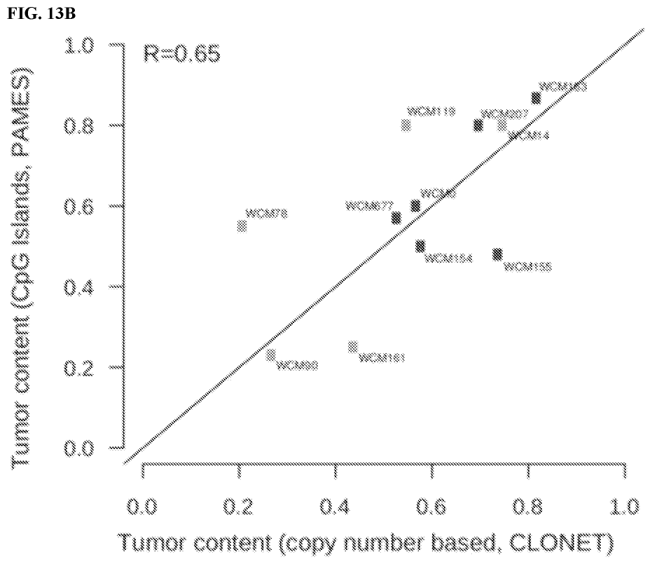

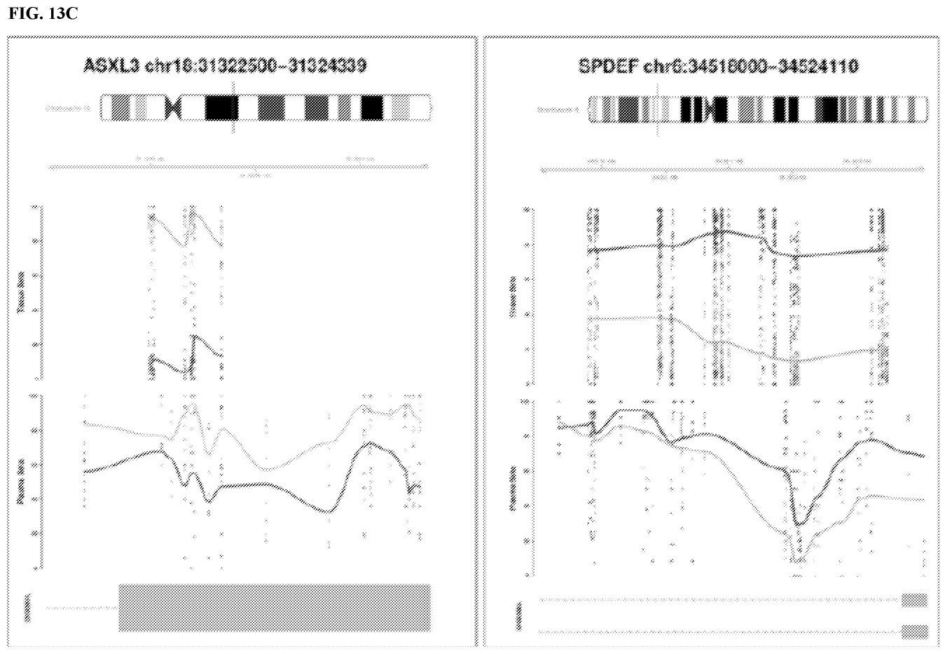

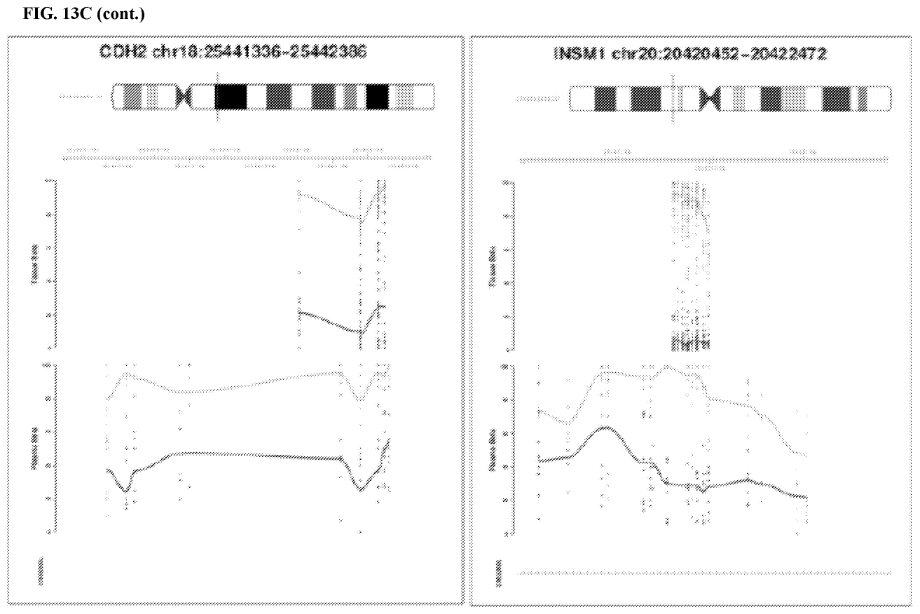

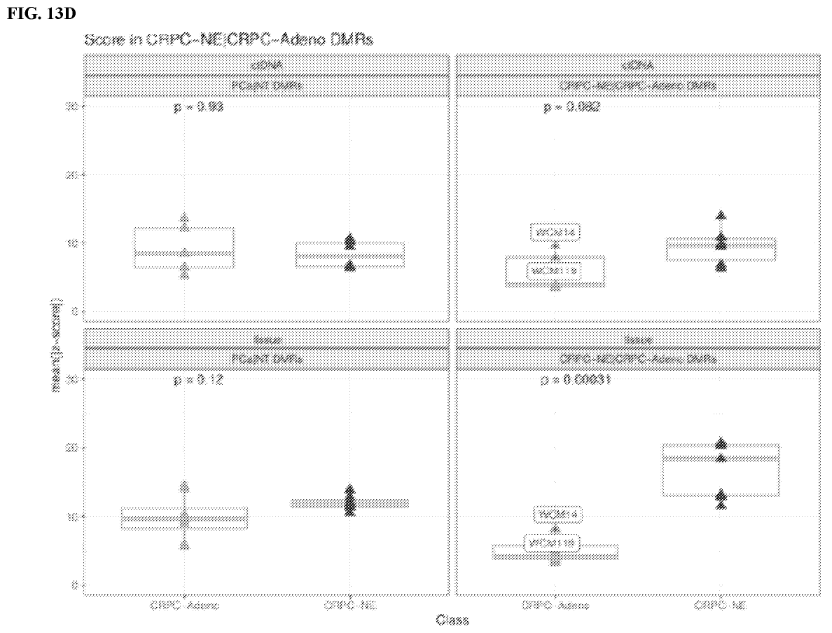

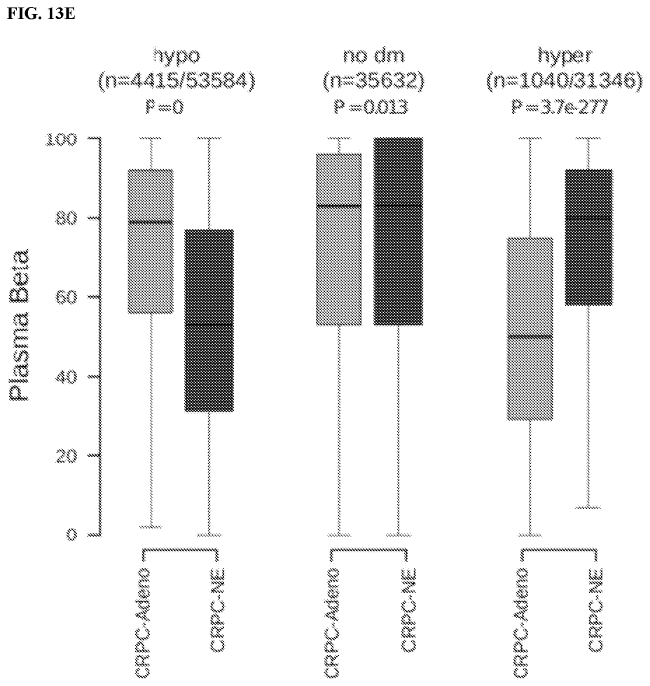

Epigenetic variability contributes to the diversity of phenotypes observed in cancer and other diseases and has been shown to play a key role in differentiation and phenotypic plasticity (Feinberg (2007) Nature 447:433-440). Conserved DNA methylation patterns across metastases has also been observed in patients with lethal prostate cancer (Aryee et al. (2013) Sci Transl Med 5:169ra110), supporting relative stability of DNA methylation patterns at least in late stage disease. Significant differences between DNA methylation patterns of CRPC-NE tissues compared to CRPC-Adeno had previously been identified (Beltran et al. (2016) Nat Med 22:298-305). In order to see if these changes are also captured by cfDNA, whole genome bisulfite sequencing of cfDNA was performed from five patients with CRPC-Adeno and six patients with CRPC-NE and compared these methylation profiles with those of their matched tumor biopsies (Table 5). Inference of cfDNA tumor content by DNA methylation using a methodology previously described (Benelli, Romagnoli, and Demichelis (2018) Bioinformatics 34:1642-1649) revealed no differences between CRPC-Adeno and CRPC-NE and was consistent with copy number-based tumor content data from WES of the same samples (FIG. 12A and FIG. 13B). Comparative analysis between plasma and biopsy tissue methylome data genome-wide (FIG. 12C and FIG. 13A) demonstrated overall concordance of differentially methylated regions in CRPC-NE (both hypo-methylated and hyper-methylated sites as shown in FIG. 13E). This included methylation of specific neuroendocrine prostate cancer classifier genes reported in Beltran et al. (Benelli, Romagnoli, and Demichelis (2018) Bioinformatics 34:1642-1649) such as ASXL3 and SPDEF, as well as hypomethylation of overexpressed CRPC-NE genes including the neuroendocrine marker INSM1 and the plasticity gene CDH2 (Table 7, FIG. 13C). Differentially methylated regions detectable by cfDNA were able to segregate patients with CRPC-Adeno or CRPC-NE (FIG. 12B and FIG. 13D). Clustering based on the location of metastases was not observed. This potentially indicates that cfDNA methylation patterns in the circulation are distinguishable based on their resistance subclass more so than by the anatomic sites of metastases within a given patient. To increase the study sample size, DNA methylation profiles were queried from a published independent cohort of 33 metastatic CRPC patients at baseline and after treatment with abiraterone acetate (Gordeviĉius et al. (2018) Clin Cancer Res 24:3317-3324).

TABLE 7

| chr | start | end | rockermeth.q_value | beta_difference | meth.state | DMR_set |
|---|---|---|---|---|---|---|
| chr1 | 833806 | 834358 | 0.003871035 | 30.95833333 | Hyper-methylated | PCa\|NT DMRs |
| chr1 | 841565 | 843221 | 0.000203452 | 48.13401361 | Hyper-methylated | PCa\|NT DMRs |
| chr1 | 861132 | 861612 | 1.20E−09 | 27.4505102 | Hyper-methylated | PCa\|NT DMRs |
| chr1 | 863044 | 863496 | 0.00064571 | −46.4047619 | Hypo-methylated | PCa\|NT DMRs |
| chr1 | 868475 | 869290 | 0.000553051 | −36.01150794 | Hypo-methylated | PCa\|NT DMRs |
| chr1 | 906156 | 907273 | 2.05E−05 | 24.38439716 | Hyper-methylated | PCa\|NT DMRs |
| chr1 | 932306 | 934055 | 1.33E−12 | 32.65304233 | Hyper-methylated | PCa\|NT DMRs |
| chr1 | 975466 | 975799 | 1.69E−08 | 39.06122449 | Hyper-methylated | PCa\|NT DMRs |
| chr1 | 986064 | 986804 | 5.92E−05 | −36.47833333 | Hypo-methylated | PCa\|NT DMRs |
| chr1 | 997828 | 997902 | 0.000205174 | 24.12202381 | Hyper-methylated | PCa\|NT DMRs |
| chr1 | 998341 | 998576 | 0.007560755 | −24.07671958 | Hypo-methylated | PCa\|NT DMRs |
| chr1 | 1009258 | 1009572 | 0.000783791 | −42.96285714 | Hypo-methylated | PCa\|NT DMRs |
| chr1 | 1108955 | 1109833 | 1.67E−11 | 24.57083333 | Hyper-methylated | PCa\|NT DMRs |
| chr1 | 1117473 | 1118863 | 3.20E−08 | 29.47852814 | Hyper-methylated | PCa\|NT DMRs |
| chr1 | 1168821 | 1169484 | 8.05E−08 | −40.97532468 | Hypo-methylated | PCa\|NT DMRs |
| chr1 | 1173013 | 1173360 | 2.78E−08 | −27.97893773 | Hypo-methylated | PCa\|NT DMRs |
| chr1 | 1181371 | 1182659 | 1.84E−19 | 32.66869301 | Hyper-methylated | PCa\|NT DMRs |
| chr1 | 1207459 | 1208406 | 0.003530139 | −28.29329004 | Hypo-methylated | PCa\|NT DMRs |
| chr1 | 1216835 | 1217837 | 1.70E−05 | −39.23003663 | Hypo-methylated | PCa\|NT DMRs |
| chr1 | 1223108 | 1227515 | 2.06E−06 | −21.73966318 | Hypo-methylated | PCa\|NT DMRs |
| chr1 | 1240237 | 1241791 | 1.34E−05 | 30.36530612 | Hyper-methylated | PCa\|NT DMRs |
| chr1 | 1244638 | 1245339 | 0.000279338 | −23.22832723 | Hypo-methylated | PCa\|NT DMRs |
| chr1 | 1289686 | 1290529 | 1.75E−12 | 25.99252874 | Hyper-methylated | PCa\|NT DMRs |
| chr1 | 1306954 | 1307443 | 0.002843063 | 26.42261905 | Hyper-methylated | PCa\|NT DMRs |
| chr1 | 1376954 | 1377657 | 0.005968871 | −22.06084656 | Hypo-methylated | PCa\|NT DMRs |
| chr1 | 1395851 | 1397240 | 2.04E−05 | 29.15826331 | Hyper-methylated | PCa\|NT DMRs |
| chr1 | 1408204 | 1408364 | 6.42E−05 | −43.0952381 | Hypo-methylated | PCa\|NT DMRs |
| chr1 | 1444231 | 1444417 | 0.005968871 | 34.17142857 | Hyper-methylated | PCa\|NT DMRs |
| chr1 | 1449165 | 1449332 | 5.94E−06 | −22.76493506 | Hypo-methylated | PCa\|NT DMRs |
| chr1 | 1475013 | 1475956 | 1.41E−22 | 28.46745027 | Hyper-methylated | PCa\|NT DMRs |
| chr1 | 1510744 | 1519232 | 0.001478325 | −23.87453416 | Hypo-methylated | PCa\|NT DMRs |
| chr1 | 1551815 | 1552238 | 0.006190613 | −30.71607143 | Hypo-methylated | PCa\|NT DMRs |
| chr1 | 1602789 | 1602925 | 0.001969641 | −36.1484127 | Hypo-methylated | PCa\|NT DMRs |
| chr1 | 1606890 | 1609822 | 2.20E−10 | 30.65119048 | Hyper-methylated | PCa\|NT DMRs |
| chr1 | 1689453 | 1691452 | 5.02E−10 | −37.8034632 | Hypo-methylated | PCa\|NT DMRs |
| chr1 | 1695797 | 1697576 | 2.42E−05 | 42.46290727 | Hyper-methylated | PCa\|NT DMRs |
| chr1 | 1823380 | 1823484 | 0.000231104 | 26.3 | Hyper-methylated | PCa\|NT DMRs |
| chr1 | 1825861 | 1826228 | 0.005968871 | −51.40952381 | Hypo-methylated | PCa\|NT DMRs |
| chr1 | 1830581 | 1831496 | 0.000570576 | 36.02486772 | Hyper-methylated | PCa\|NT DMRs |
| chr1 | 1950415 | 1950521 | 7.44E−06 | 24.18095238 | Hyper-methylated | PCa\|NT DMRs |
| chr1 | 1978540 | 1979100 | 0.00051204 | 27.80238095 | Hyper-methylated | PCa\|NT DMRs |
| chr1 | 2013800 | 2014245 | 0.005968871 | −60.71714286 | Hypo-methylated | PCa\|NT DMRs |
| chr1 | 2057319 | 2058636 | 0.000411236 | 28.56613757 | Hyper-methylated | PCa\|NT DMRs |
| chr1 | 2075932 | 2082443 | 0.000226774 | 45.09238095 | Hyper-methylated | PCa\|NT DMRs |
| chr1 | 2121111 | 2124399 | 5.90E−08 | −23.57375661 | Hypo-methylated | PCa\|NT DMRs |
| chr1 | 2136909 | 2137665 | 2.48E−08 | 25.92175824 | Hyper-methylated | PCa\|NT DMRs |
| chr1 | 2144913 | 2144998 | 8.17E−05 | 21.11688312 | Hyper-methylated | PCa\|NT DMRs |
| chr1 | 2222218 | 2223007 | 0.000860601 | 37.41385281 | Hyper-methylated | PCa\|NT DMRs |
| chr1 | 2230768 | 2233721 | 3.18E−10 | 40.87057387 | Hyper-methylated | PCa\|NT DMRs |
| chr1 | 2253541 | 2253851 | 0.000249628 | 20.55952381 | Hyper-methylated | PCa\|NT DMRs |
| chr1 | 2254128 | 2255441 | 8.84E−09 | −22.32430213 | Hypo-methylated | PCa\|NT DMRs |
| chr1 | 2273416 | 2273610 | 0.00179283 | −25.16071429 | Hypo-methylated | PCa\|NT DMRs |
| chr1 | 2335963 | 2337519 | 2.40E−06 | −23.93361345 | Hypo-methylated | PCa\|NT DMRs |
| chr1 | 2391010 | 2391691 | 2.67E−05 | −39.64217687 | Hypo-methylated | PCa\|NT DMRs |
| chr1 | 2398952 | 2399054 | 0.00064571 | 23.12585034 | Hyper-methylated | PCa\|NT DMRs |
| chr1 | 2496056 | 2497941 | 0.001083007 | −22.66 | Hypo-methylated | PCa\|NT DMRs |
| chr1 | 2541092 | 2541937 | 0.000696555 | −29.35324675 | Hypo-methylated | PCa\|NT DMRs |
| chr1 | 2564998 | 2569756 | 1.67E−05 | −26.14484127 | Hypo-methylated | PCa\|NT DMRs |
| chr1 | 2889964 | 2890447 | 0.000481535 | 20.03703704 | Hyper-methylated | PCa\|NT DMRs |
| chr1 | 2987330 | 2988549 | 1.24E−09 | 24.68901099 | Hyper-methylated | PCa\|NT DMRs |
| chr1 | 3230300 | 3237142 | 4.87E−07 | −33.46066253 | Hypo-methylated | PCa\|NT DMRs |
| chr1 | 3251914 | 3252004 | 0.008482405 | −69.11428571 | Hypo-methylated | PCa\|NT DMRs |
| chr1 | 3255907 | 3263385 | 4.71E−07 | −29.36917293 | Hypo-methylated | PCa\|NT DMRs |
| chr1 | 3265179 | 3266823 | 1.62E−10 | −30.5044733 | Hypo-methylated | PCa\|NT DMRs |
| chr1 | 3321503 | 3325095 | 1.56E−15 | −25.76432607 | Hypo-methylated | PCa\|NT DMRs |
| chr1 | 3397205 | 3399514 | 0.000680799 | 20.16026786 | Hyper-methylated | PCa\|NT DMRs |
| chr1 | 3412502 | 3412634 | 0.000858283 | 30.4969697 | Hyper-methylated | PCa\|NT DMRs |
| chr1 | 3432220 | 3436195 | 0.000118733 | 20.72708995 | Hyper-methylated | PCa\|NT DMRs |
| chr1 | 3454859 | 3455550 | 7.57E−05 | 41.04166667 | Hyper-methylated | PCa\|NT DMRs |
| chr1 | 3470175 | 3470440 | 0.00064571 | −43.92176871 | Hypo-methylated | PCa\|NT DMRs |
| chr1 | 3496497 | 3497778 | 0.000286886 | −20.83766234 | Hypo-methylated | PCa\|NT DMRs |
| chr1 | 3498249 | 3498791 | 3.66E−05 | −27.54333333 | Hypo-methylated | PCa\|NT DMRs |
| chr1 | 3503996 | 3504148 | 0.001969641 | −53 | Hypo-methylated | PCa\|NT DMRs |
| chr1 | 3593716 | 3595670 | 8.24E−05 | 35.22134647 | Hyper-methylated | PCa\|NT DMRs |
| chr1 | 3688353 | 3688626 | 0.003544378 | 31.35238095 | Hyper-methylated | PCa\|NT DMRs |
| chr1 | 3793061 | 3797937 | 0.009049402 | 22.2 | Hyper-methylated | PCa\|NT DMRs |
| chr1 | 3807161 | 3808428 | 0.000117699 | 21.90692641 | Hyper-methylated | PCa\|NT DMRs |
| chr1 | 3832884 | 4005884 | 1.48E−10 | −27.74664723 | Hypo-methylated | PCa\|NT DMRs |
| chr1 | 4008234 | 4058997 | 2.33E−18 | −35.05553571 | Hypo-methylated | PCa\|NT DMRs |
| chr1 | 4060225 | 4087186 | 2.81E−19 | −35.11046058 | Hypo-methylated | PCa\|NT DMRs |
| chr1 | 4093526 | 4117451 | 1.68E−07 | −34.3547619 | Hypo-methylated | PCa\|NT DMRs |
| chr1 | 4587426 | 4593526 | 5.84E−05 | −43.68315018 | Hypo-methylated | PCa\|NT DMRs |
| chr1 | 4722530 | 4736275 | 2.56E−05 | −26.49188713 | Hypo-methylated | PCa\|NT DMRs |
| chr1 | 4790174 | 4794300 | 0.000768419 | −39.51465201 | Hypo-methylated | PCa\|NT DMRs |
| chr1 | 4829849 | 4832644 | 2.81E−09 | −29.51684982 | Hypo-methylated | PCa\|NT DMRs |
| chr1 | 4851218 | 4995869 | 3.41E−11 | −34.58172757 | Hypo-methylated | PCa\|NT DMRs |
| chr1 | 5001929 | 5046353 | 1.31E−13 | −28.73313764 | Hypo-methylated | PCa\|NT DMRs |
| chr1 | 5335385 | 5554029 | 1.49E−45 | −30.9928836 | Hypo-methylated | PCa\|NT DMRs |
| chr1 | 5676228 | 5676419 | 6.42E−05 | −25.62962963 | Hypo-methylated | PCa\|NT DMRs |
| chr1 | 5712462 | 5745743 | 0.000240645 | −23.53783784 | Hypo-methylated | PCa\|NT DMRs |
| chr1 | 5795237 | 5805262 | 0.001480644 | −26.75571429 | Hypo-methylated | PCa\|NT DMRs |
| chr1 | 5814791 | 5829233 | 3.20E−14 | −24.03341837 | Hypo-methylated | PCa\|NT DMRs |
| chr1 | 5901850 | 5905277 | 0.005968871 | −28.4015873 | Hypo-methylated | PCa\|NT DMRs |
| chr1 | 6008515 | 6009858 | 0.00068166 | −44.50595238 | Hypo-methylated | PCa\|NT DMRs |
| chr1 | 6046227 | 6046872 | 0.007305013 | −51.41071429 | Hypo-methylated | PCa\|NT DMRs |
| chr1 | 6053341 | 6054950 | 0.003949974 | 37.8989011 | Hyper-methylated | PCa\|NT DMRs |
| chr1 | 6094117 | 6095514 | 0.000109224 | −30.61761905 | Hypo-methylated | PCa\|NT DMRs |
| chr1 | 6142719 | 6142965 | 0.005968871 | 25.15619048 | Hyper-methylated | PCa\|NT DMRs |
| chr1 | 6155169 | 6155484 | 0.001065506 | −30.27472527 | Hypo-methylated | PCa\|NT DMRs |
| chr1 | 6162700 | 6166714 | 7.82E−07 | −22.00401003 | Hypo-methylated | PCa\|NT DMRs |
| chr1 | 6230825 | 6231135 | 0.001850915 | 31.95714286 | Hyper-methylated | PCa\|NT DMRs |
| chr1 | 6280441 | 6283157 | 0.002765491 | −24.67380952 | Hypo-methylated | PCa\|NT DMRs |
| chr1 | 6391527 | 6392259 | 0.002291701 | −22.32440476 | Hypo-methylated | PCa\|NT DMRs |
| chr1 | 6474659 | 6479466 | 1.22E−09 | 20.17793975 | Hyper-methylated | PCa\|NT DMRs |
| chr1 | 6487398 | 6487656 | 0.007959104 | −23.10204082 | Hypo-methylated | PCa\|NT DMRs |
| chr1 | 6525502 | 6526718 | 0.000205174 | 43.21428571 | Hyper-methylated | PCa\|NT DMRs |
| chr1 | 6569576 | 6570371 | 0.00090274 | 33.7485119 | Hyper-methylated | PCa\|NT DMRs |
| chr1 | 6615084 | 6615539 | 0.000145094 | −31.57222222 | Hypo-methylated | PCa\|NT DMRs |
| chr1 | 6663334 | 6663683 | 1.00E−06 | 22.03007519 | Hyper-methylated | PCa\|NT DMRs |
| chr1 | 6665369 | 6672507 | 0.007679542 | −32.08095238 | Hypo-methylated | PCa\|NT DMRs |
| chr1 | 7129988 | 7130188 | 0.003742662 | 22.55904762 | Hyper-methylated | PCa\|NT DMRs |
| chr1 | 7154719 | 7187123 | 4.24E−10 | −26.76927438 | Hypo-methylated | PCa\|NT DMRs |
| chr1 | 7281456 | 7290018 | 4.45E−08 | −25.94126984 | Hypo-methylated | PCa\|NT DMRs |
| chr1 | 7393386 | 7393754 | 0.000117699 | −38.36363636 | Hypo-methylated | PCa\|NT DMRs |
| chr1 | 7496966 | 7500717 | 3.18E−06 | −23.19467787 | Hypo-methylated | PCa\|NT DMRs |
| chr1 | 7515592 | 7515823 | 0.00064571 | −47.26530612 | Hypo-methylated | PCa\|NT DMRs |
| chr1 | 7523160 | 7523237 | 0.005968871 | −33.2 | Hypo-methylated | PCa\|NT DMRs |
| chr1 | 7551243 | 7551524 | 0.004095673 | −45.36111111 | Hypo-methylated | PCa\|NT DMRs |
| chr1 | 7593169 | 7593260 | 0.001969641 | −28.16666667 | Hypo-methylated | PCa\|NT DMRs |
| chr1 | 7605939 | 7624371 | 6.90E−10 | −22.0377551 | Hypo-methylated | PCa\|NT DMRs |
| chr1 | 7634567 | 7634752 | 0.000474737 | −55.3015873 | Hypo-methylated | PCa\|NT DMRs |
| chr1 | 7669905 | 7676554 | 0.000149331 | −26.4755102 | Hypo-methylated | PCa\|NT DMRs |
| chr1 | 7687895 | 7690531 | 0.00010546 | −22.39904762 | Hypo-methylated | PCa\|NT DMRs |
| chr1 | 7693634 | 7696223 | 2.38E−07 | −24.58658009 | Hypo-methylated | PCa\|NT DMRs |
| chr1 | 7923960 | 7944257 | 0.000145094 | −26.3015873 | Hypo-methylated | PCa\|NT DMRs |
| chr1 | 8065016 | 8065134 | 0.008482405 | −49.09904762 | Hypo-methylated | PCa\|NT DMRs |
| chr1 | 8270840 | 8302412 | 1.20E−07 | 26.88635913 | Hyper-methylated | PCa\|NT DMRs |
| chr1 | 8336576 | 8343765 | 6.54E−05 | 27.20308123 | Hyper-methylated | PCa\|NT DMRs |
| chr1 | 8389605 | 8389970 | 0.000205174 | 24.78571429 | Hyper-methylated | PCa\|NT DMRs |
| chr1 | 8403834 | 8404051 | 0.00064571 | −62.47278912 | Hypo-methylated | PCa\|NT DMRs |
| chr1 | 8409842 | 8421025 | 4.98E−09 | −21.11657848 | Hypo-methylated | PCa\|NT DMRs |
| chr1 | 8421680 | 8433942 | 0.001582756 | −35.61754386 | Hypo-methylated | PCa\|NT DMRs |
| chr1 | 8721541 | 8745653 | 0.000803706 | −24.34962406 | Hypo-methylated | PCa\|NT DMRs |
| chr1 | 8968659 | 8968724 | 0.00023371 | 39.3 | Hyper-methylated | PCa\|NT DMRs |
| chr1 | 9124451 | 9127247 | 0.007239577 | 31.52793651 | Hyper-methylated | PCa\|NT DMRs |
| chr1 | 9293230 | 9293645 | 1.00E−05 | 38.64761905 | Hyper-methylated | PCa\|NT DMRs |
| chr1 | 9310824 | 9312074 | 2.58E−05 | −36.94196429 | Hypo-methylated | PCa\|NT DMRs |
| chr1 | 9378117 | 9378377 | 0.002094 | 25.04960317 | Hyper-methylated | PCa\|NT DMRs |
| chr1 | 9399002 | 9399325 | 0.000483473 | −37.71854637 | Hypo-methylated | PCa\|NT DMRs |
| chr1 | 9520277 | 9527533 | 0.000103673 | −45.01011905 | Hypo-methylated | PCa\|NT DMRs |
| chr1 | 9601503 | 9621111 | 1.91E−06 | −27.1378882 | Hypo-methylated | PCa\|NT DMRs |
| chr1 | 9666694 | 9667637 | 0.001291665 | −32.77705628 | Hypo-methylated | PCa\|NT DMRs |
| chr1 | 9688558 | 9691043 | 3.06E−06 | 38.84380952 | Hyper-methylated | PCa\|NT DMRs |
| chr1 | 9715244 | 9715398 | 0.001969641 | 39.53333333 | Hyper-methylated | PCa\|NT DMRs |
| chr1 | 9777564 | 9777967 | 0.001969641 | 40.07619048 | Hyper-methylated | PCa\|NT DMRs |
| chr1 | 9909812 | 9911364 | 0.003871035 | 37.87261905 | Hyper-methylated | PCa\|NT DMRs |
| chr1 | 9935455 | 9935545 | 0.000377317 | −25.625 | Hypo-methylated | PCa\|NT DMRs |
| chr1 | 10511545 | 10511597 | 0.000377317 | 26.43214286 | Hyper-methylated | PCa\|NT DMRs |
| chr1 | 10700503 | 10701575 | 0.001139334 | −26.98928571 | Hypo-methylated | PCa\|NT DMRs |
| chr1 | 10702182 | 10708045 | 1.60E−11 | −30.99710145 | Hypo-methylated | PCa\|NT DMRs |
| chr1 | 10717177 | 10724769 | 4.64E−10 | −22.26821572 | Hypo-methylated | PCa\|NT DMRs |
| chr1 | 10732160 | 10734473 | 0.002771971 | −30.43508772 | Hypo-methylated | PCa\|NT DMRs |
| chr1 | 10749937 | 10750080 | 0.004095673 | −46.28571429 | Hypo-methylated | PCa\|NT DMRs |
| chr1 | 10776393 | 10776855 | 0.005542011 | 44.98051948 | Hyper-methylated | PCa\|NT DMRs |
| chr1 | 10795360 | 10795472 | 0.005438982 | 25.8647619 | Hyper-methylated | PCa\|NT DMRs |
| chr1 | 10816475 | 10822115 | 0.00044387 | −34.77328042 | Hypo-methylated | PCa\|NT DMRs |
| chr1 | 10834251 | 10843155 | 0.000753029 | −28.68701299 | Hypo-methylated | PCa\|NT DMRs |
| chr1 | 10889510 | 10895896 | 0.000426728 | 21.09079365 | Hyper-methylated | PCa\|NT DMRs |
| chr1 | 10909319 | 10916649 | 0.002097193 | −27.71428571 | Hypo-methylated | PCa\|NT DMRs |
| chr1 | 10958562 | 10959556 | 0.001201396 | 34.46571429 | Hyper-methylated | PCa\|NT DMRs |
| chr1 | 10960174 | 10961646 | 0.000376662 | −20.27553366 | Hypo-methylated | PCa\|NT DMRs |
| chr1 | 10963986 | 10965396 | 2.87E−06 | 43.06011905 | Hyper-methylated | PCa\|NT DMRs |
| chr1 | 11118847 | 11119226 | 6.93E−06 | −49.33492063 | Hypo-methylated | PCa\|NT DMRs |
| chr1 | 11323251 | 11323468 | 0.005968871 | −33.84761905 | Hypo-methylated | PCa\|NT DMRs |
| chr1 | 11645732 | 11645979 | 0.003871035 | −20.53373016 | Hypo-methylated | PCa\|NT DMRs |
| chr1 | 11714280 | 11714430 | 8.62E−05 | 20.80952381 | Hyper-methylated | PCa\|NT DMRs |
| chr1 | 11752120 | 11752816 | 6.42E−13 | 29.48321624 | Hyper-methylated | PCa\|NT DMRs |
| chr1 | 11768133 | 11773620 | 0.001969641 | −20.33469388 | Hypo-methylated | PCa\|NT DMRs |
| chr1 | 11809720 | 11812628 | 0.001039378 | −20.08791209 | Hypo-methylated | PCa\|NT DMRs |
| chr1 | 11849426 | 11851296 | 0.002437742 | −38.38359788 | Hypo-methylated | PCa\|NT DMRs |
| chr1 | 11863236 | 11863430 | 1.97E−05 | 20.24285714 | Hyper-methylated | PCa\|NT DMRs |
| chr1 | 11919349 | 11919404 | 0.000145171 | 24.8877551 | Hyper-methylated | PCa\|NT DMRs |
| chr1 | 11958191 | 11958408 | 4.02E−05 | 24.63386243 | Hyper-methylated | PCa\|NT DMRs |
| chr1 | 11979113 | 11979209 | 0.001969641 | −49.3952381 | Hypo-methylated | PCa\|NT DMRs |
| chr1 | 12185599 | 12186259 | 0.000354065 | 30.15978836 | Hyper-methylated | PCa\|NT DMRs |
| chr1 | 12202241 | 12226851 | 0.000703976 | 20.01578231 | Hyper-methylated | PCa\|NT DMRs |
| chr1 | 12227265 | 12227921 | 6.08E−15 | 33.04055829 | Hyper-methylated | PCa\|NT DMRs |
| chr1 | 12239199 | 12240922 | 0.000205174 | 36.74089636 | Hyper-methylated | PCa\|NT DMRs |
| chr1 | 12245242 | 12245615 | 0.000117699 | 40.22467532 | Hyper-methylated | PCa\|NT DMRs |
| chr1 | 12248895 | 12251890 | 0.009069241 | −27.87012987 | Hypo-methylated | PCa\|NT DMRs |
| chr1 | 12655157 | 12655322 | 0.000481535 | 23.71428571 | Hyper-methylated | PCa\|NT DMRs |
| chr1 | 13774065 | 13789631 | 0.001836557 | −32.32967033 | Hypo-methylated | PCa\|NT DMRs |
| chr1 | 13909632 | 13909702 | 0.000908257 | 21.67032967 | Hyper-methylated | PCa\|NT DMRs |
| chr1 | 14227615 | 14228198 | 1.76E−06 | −53.49285714 | Hypo-methylated | PCa\|NT DMRs |
| chr1 | 15100185 | 15105936 | 0.002770271 | 21.63736264 | Hyper-methylated | PCa\|NT DMRs |
| chr1 | 15271860 | 15273458 | 0.00051204 | −45.00952381 | Hypo-methylated | PCa\|NT DMRs |
| chr1 | 15481103 | 15481562 | 1.63E−05 | 37.9 | Hyper-methylated | PCa\|NT DMRs |
| chr1 | 15672607 | 15672976 | 0.000513415 | 29.87142857 | Hyper-methylated | PCa\|NT DMRs |
| chr1 | 15711358 | 15713101 | 0.001241245 | 29.48994709 | Hyper-methylated | PCa\|NT DMRs |
| chr1 | 15738251 | 15742694 | 0.002225983 | 39.50952381 | Hyper-methylated | PCa\|NT DMRs |
| chr1 | 15772100 | 15772171 | 0.005968871 | 42.2 | Hyper-methylated | PCa\|NT DMRs |
| chr1 | 16124698 | 16126472 | 0.00064571 | −42.75986395 | Hypo-methylated | PCa\|NT DMRs |
| chr1 | 16163269 | 16166996 | 0.000473059 | 32.10666667 | Hyper-methylated | PCa\|NT DMRs |
| chr1 | 16266065 | 16268625 | 0.001097219 | −28.03753501 | Hypo-methylated | PCa\|NT DMRs |
| chr1 | 16276092 | 16278114 | 0.000396946 | 27.61458333 | Hyper-methylated | PCa\|NT DMRs |
| chr1 | 16471331 | 16472685 | 4.70E−07 | −41.0031746 | Hypo-methylated | PCa\|NT DMRs |
| chr1 | 16480820 | 16481019 | 0.000974795 | 30.35714286 | Hyper-methylated | PCa\|NT DMRs |
| chr1 | 16488561 | 16489314 | 2.17E−07 | 37.47063492 | Hyper-methylated | PCa\|NT DMRs |
| chr1 | 16542988 | 16543568 | 9.66E−07 | 21.66190476 | Hyper-methylated | PCa\|NT DMRs |
| chr1 | 16553411 | 16554461 | 1.60E−19 | 33.56600733 | Hyper-methylated | PCa\|NT DMRs |
| chr1 | 16638575 | 16643635 | 0.000783791 | −33.28285714 | Hypo-methylated | PCa\|NT DMRs |
| chr1 | 16847234 | 16847864 | 1.14E−05 | 26.69382716 | Hyper-methylated | PCa\|NT DMRs |
| chr1 | 16950871 | 16951184 | 0.002534928 | −37.63142857 | Hypo-methylated | PCa\|NT DMRs |
| chr1 | 16974210 | 16974916 | 6.42E−05 | 27.68677249 | Hyper-methylated | PCa\|NT DMRs |
| chr1 | 17305366 | 17305831 | 0.000166226 | 38.36043956 | Hyper-methylated | PCa\|NT DMRs |
| chr1 | 17445283 | 17445511 | 5.94E−06 | 38.74458874 | Hyper-methylated | PCa\|NT DMRs |
| chr1 | 17446145 | 17448659 | 0.005178633 | 20.08787879 | Hyper-methylated | PCa\|NT DMRs |
| chr1 | 17490697 | 17511836 | 0.000688325 | −28.99450549 | Hypo-methylated | PCa\|NT DMRs |
| chr1 | 17715983 | 17719078 | 0.000226106 | −21.07838828 | Hypo-methylated | PCa\|NT DMRs |
| chr1 | 17773133 | 17787034 | 0.005968871 | −29.74166667 | Hypo-methylated | PCa\|NT DMRs |
| chr1 | 17893243 | 17896312 | 0.001969641 | 26.35238095 | Hyper-methylated | PCa\|NT DMRs |
| chr1 | 17906181 | 17907373 | 0.00248539 | 20.33571429 | Hyper-methylated | PCa\|NT DMRs |
| chr1 | 18043398 | 18043467 | 0.00064571 | −21.89795918 | Hypo-methylated | PCa\|NT DMRs |
| chr1 | 18117942 | 18127214 | 8.73E−07 | −22.19979296 | Hypo-methylated | PCa\|NT DMRs |
| chr1 | 18348682 | 18384743 | 0.007428376 | −23.71978022 | Hypo-methylated | PCa\|NT DMRs |
| chr1 | 18482237 | 18483343 | 9.81E−07 | −44.39206349 | Hypo-methylated | PCa\|NT DMRs |
| chr1 | 18869064 | 18879613 | 0.007526322 | 25.11833333 | Hyper-methylated | PCa\|NT DMRs |
| chr1 | 18976852 | 18988808 | 0.008319869 | 20.97943723 | Hyper-methylated | PCa\|NT DMRs |
| chr1 | 19000796 | 19002350 | 6.42E−05 | 27.48941799 | Hyper-methylated | PCa\|NT DMRs |
| chr1 | 19010192 | 19011587 | 0.000324616 | 25.0535014 | Hyper-methylated | PCa\|NT DMRs |
| chr1 | 19144335 | 19149038 | 0.000114755 | 28.72547619 | Hyper-methylated | PCa\|NT DMRs |
| chr1 | 19201690 | 19202214 | 0.000104435 | −22.7478458 | Hypo-methylated | PCa\|NT DMRs |
| chr1 | 19717467 | 19717634 | 0.005968871 | 50.66190476 | Hyper-methylated | PCa\|NT DMRs |
| chr1 | 19739247 | 19739501 | 0.008482405 | −64.1047619 | Hypo-methylated | PCa\|NT DMRs |
| chr1 | 20081194 | 20081341 | 0.001969641 | −49.8468254 | Hypo-methylated | PCa\|NT DMRs |
| chr1 | 20491304 | 20495238 | 1.65E−07 | −34.76848073 | Hypo-methylated | PCa\|NT DMRs |
| chr1 | 20512064 | 20517701 | 5.31E−06 | 36.78095238 | Hyper-methylated | PCa\|NT DMRs |
| chr1 | 20617283 | 20637260 | 0.000109356 | 34.33142857 | Hyper-methylated | PCa\|NT DMRs |
| chr1 | 21056502 | 21058361 | 4.40E−10 | 29.44976959 | Hyper-methylated | PCa\|NT DMRs |
| chr1 | 21589014 | 21595186 | 4.69E−05 | −23.76890756 | Hypo-methylated | PCa\|NT DMRs |
| chr1 | 21652411 | 21652493 | 0.005968871 | 41.48571429 | Hyper-methylated | PCa\|NT DMRs |
| chr1 | 21860580 | 21861981 | 0.00641359 | −28.11904762 | Hypo-methylated | PCa\|NT DMRs |
| chr1 | 21864320 | 21865088 | 1.52E−06 | 31.16507937 | Hyper-methylated | PCa\|NT DMRs |
| chr1 | 21877537 | 21877782 | 0.000205174 | −35.13888889 | Hypo-methylated | PCa\|NT DMRs |
| chr1 | 21950270 | 21953521 | 0.000168936 | −35.44805195 | Hypo-methylated | PCa\|NT DMRs |
| chr1 | 21960463 | 21966546 | 0.002285803 | −26.07428571 | Hypo-methylated | PCa\|NT DMRs |
| chr1 | 21978653 | 21979288 | 0.001394173 | 26.67176871 | Hyper-methylated | PCa\|NT DMRs |
| chr1 | 22149879 | 22152072 | 0.002856529 | 23.73154762 | Hyper-methylated | PCa\|NT DMRs |
| chr1 | 22350910 | 22351261 | 7.95E−05 | 23.02930403 | Hyper-methylated | PCa\|NT DMRs |
| chr1 | 22535198 | 22535512 | 0.007679542 | 23.78306878 | Hyper-methylated | PCa\|NT DMRs |
| chr1 | 22576056 | 22586332 | 2.43E−07 | −43.89691877 | Hypo-methylated | PCa\|NT DMRs |
| chr1 | 22614808 | 22624308 | 0.001684481 | 23.85510204 | Hyper-methylated | PCa\|NT DMRs |
| chr1 | 22909639 | 22915043 | 0.000392715 | −21.06809524 | Hypo-methylated | PCa\|NT DMRs |
| chr1 | 22927298 | 22927533 | 0.00753722 | −24.31868132 | Hypo-methylated | PCa\|NT DMRs |
| chr1 | 22944642 | 22944694 | 0.000205174 | −29.91071429 | Hypo-methylated | PCa\|NT DMRs |
| chr1 | 22946418 | 22953022 | 0.007679542 | 25.57936508 | Hyper-methylated | PCa\|NT DMRs |
| chr1 | 23279817 | 23280300 | 2.19E−23 | 33.84963112 | Hyper-methylated | PCa\|NT DMRs |
| chr1 | 23805521 | 23805874 | 0.004095673 | −55.83333333 | Hypo-methylated | PCa\|NT DMRs |
| chr1 | 23880455 | 23881057 | 0.00216533 | 30.125 | Hyper-methylated | PCa\|NT DMRs |
| chr1 | 23918816 | 23928152 | 0.005968871 | 22.87142857 | Hyper-methylated | PCa\|NT DMRs |
| chr1 | 24120771 | 24124208 | 0.000200037 | −28.63533835 | Hypo-methylated | PCa\|NT DMRs |
| chr1 | 24127957 | 24128033 | 8.73E−05 | −37.30612245 | Hypo-methylated | PCa\|NT DMRs |
| chr1 | 24743847 | 24744307 | 0.005968871 | −43.1047619 | Hypo-methylated | PCa\|NT DMRs |
| chr1 | 24967334 | 24968562 | 0.00026034 | 23.03571429 | Hyper-methylated | PCa\|NT DMRs |
| chr1 | 25196356 | 25198876 | 5.87E−08 | 45.31738095 | Hyper-methylated | PCa\|NT DMRs |
| chr1 | 25327826 | 25328022 | 0.005438982 | 26.78571429 | Hyper-methylated | PCa\|NT DMRs |
| chr1 | 25346057 | 25349115 | 0.008581782 | 33.56547619 | Hyper-methylated | PCa\|NT DMRs |
| chr1 | 25568119 | 25572539 | 0.00068166 | −45.07738095 | Hypo-methylated | PCa\|NT DMRs |
| chr1 | 25882610 | 25890156 | 9.62E−05 | 21.5764881 | Hyper-methylated | PCa\|NT DMRs |
| chr1 | 25894889 | 25895652 | 6.42E−05 | −55.24338624 | Hypo-methylated | PCa\|NT DMRs |
| chr1 | 25944228 | 25944662 | 9.07E−08 | 22.14285714 | Hyper-methylated | PCa\|NT DMRs |
| chr1 | 26075447 | 26075643 | 0.005968871 | −44.8 | Hypo-methylated | PCa\|NT DMRs |
| chr1 | 26202251 | 26202628 | 1.70E−11 | 22.82014652 | Hyper-methylated | PCa\|NT DMRs |
| chr1 | 26317172 | 26319385 | 0.003587573 | 22.26493506 | Hyper-methylated | PCa\|NT DMRs |
| chr1 | 26372021 | 26372155 | 0.005375157 | 20.13265306 | Hyper-methylated | PCa\|NT DMRs |
| chr1 | 26481932 | 26482761 | 0.001241245 | 27.58730159 | Hyper-methylated | PCa\|NT DMRs |
| chr1 | 26503847 | 26504147 | 4.45E−08 | −21.9352381 | Hypo-methylated | PCa\|NT DMRs |
| chr1 | 26614241 | 26617546 | 2.86E−05 | 51.36190476 | Hyper-methylated | PCa\|NT DMRs |
| chr1 | 26645629 | 26645803 | 0.005968871 | 33.88571429 | Hyper-methylated | PCa\|NT DMRs |
| chr1 | 26671747 | 26684107 | 0.000822388 | −21.46513057 | Hypo-methylated | PCa\|NT DMRs |
| chr1 | 26687175 | 26687430 | 5.94E−06 | 20.11255411 | Hyper-methylated | PCa\|NT DMRs |
| chr1 | 26848240 | 26848450 | 0.001969641 | −21.7014652 | Hypo-methylated | PCa\|NT DMRs |
| chr1 | 27131671 | 27131723 | 0.001969641 | 42.61904762 | Hyper-methylated | PCa\|NT DMRs |
| chr1 | 27338911 | 27339194 | 0.000207281 | 29.16135531 | Hyper-methylated | PCa\|NT DMRs |
| chr1 | 27687140 | 27687696 | 2.45E−16 | 47.72445055 | Hyper-methylated | PCa\|NT DMRs |
| chr1 | 27729855 | 27730377 | 5.52E−05 | 33.27229437 | Hyper-methylated | PCa\|NT DMRs |
| chr1 | 27840125 | 27840800 | 0.001480644 | 32.49952381 | Hyper-methylated | PCa\|NT DMRs |
| chr1 | 27846977 | 27848263 | 0.007338757 | 43.20535714 | Hyper-methylated | PCa\|NT DMRs |
| chr1 | 27882335 | 27882652 | 0.006466965 | 30.18367347 | Hyper-methylated | PCa\|NT DMRs |
| chr1 | 27923968 | 27925971 | 0.001969641 | 40.27619048 | Hyper-methylated | PCa\|NT DMRs |
| chr1 | 27939500 | 27941783 | 0.000981814 | −24.03961905 | Hypo-methylated | PCa\|NT DMRs |
| chr1 | 28013247 | 28013387 | 0.001969641 | −40.35714286 | Hypo-methylated | PCa\|NT DMRs |
| chr1 | 28195587 | 28197508 | 0.009407512 | 32.57142857 | Hyper-methylated | PCa\|NT DMRs |
| chr1 | 28500721 | 28501881 | 0.00064571 | 23.29591837 | Hyper-methylated | PCa\|NT DMRs |
| chr1 | 29075805 | 29075853 | 0.005968871 | 41.42857143 | Hyper-methylated | PCa\|NT DMRs |
| chr1 | 29139076 | 29139838 | 0.005433534 | −30.9047619 | Hypo-methylated | PCa\|NT DMRs |
| chr1 | 29189987 | 29197088 | 0.000491935 | −22.88756614 | Hypo-methylated | PCa\|NT DMRs |
| chr1 | 29460830 | 29461328 | 2.17E−12 | 25.55703212 | Hyper-methylated | PCa\|NT DMRs |
| chr1 | 29657461 | 29708397 | 9.63E−06 | −25.73958333 | Hypo-methylated | PCa\|NT DMRs |
| chr1 | 29721179 | 29763815 | 7.09E−05 | −22.65303777 | Hypo-methylated | PCa\|NT DMRs |
| chr1 | 29869374 | 29877760 | 0.000188647 | −27.52877847 | Hypo-methylated | PCa\|NT DMRs |
| chr1 | 30259620 | 30301673 | 7.06E−08 | −27.06077098 | Hypo-methylated | PCa\|NT DMRs |
| chr1 | 30372179 | 30375353 | 0.003192753 | −20.29391534 | Hypo-methylated | PCa\|NT DMRs |
| chr1 | 30424950 | 30444639 | 0.00064571 | −30.02721088 | Hypo-methylated | PCa\|NT DMRs |
| chr1 | 30515011 | 30538636 | 5.95E−08 | −22.51490015 | Hypo-methylated | PCa\|NT DMRs |
| chr1 | 30573155 | 30574847 | 0.000509286 | −25.13784461 | Hypo-methylated | PCa\|NT DMRs |
| chr1 | 30587658 | 30705290 | 2.34E−06 | −27.11693122 | Hypo-methylated | PCa\|NT DMRs |
| chr1 | 30720474 | 30776141 | 1.07E−09 | −24.58324515 | Hypo-methylated | PCa\|NT DMRs |
| chr1 | 30798832 | 30802077 | 0.000196301 | −27.76785714 | Hypo-methylated | PCa\|NT DMRs |
| chr1 | 30911320 | 30937802 | 0.002585277 | −35.15119048 | Hypo-methylated | PCa\|NT DMRs |
| chr1 | 31278174 | 31280213 | 0.001789867 | 20.00291005 | Hyper-methylated | PCa\|NT DMRs |
| chr1 | 31380703 | 31381102 | 7.12E−06 | 22.21256614 | Hyper-methylated | PCa\|NT DMRs |
| chr1 | 31989683 | 32003965 | 4.78E−05 | 27.38738095 | Hyper-methylated | PCa\|NT DMRs |
| chr1 | 32013367 | 32014374 | 0.007560755 | −29.82202381 | Hypo-methylated | PCa\|NT DMRs |
| chr1 | 32052628 | 32052681 | 0.00023371 | 20.6547619 | Hyper-methylated | PCa\|NT DMRs |
| chr1 | 32281534 | 32281723 | 0.005513661 | −27.69642857 | Hypo-methylated | PCa\|NT DMRs |
| chr1 | 32666528 | 32666956 | 0.001969641 | 26.86734694 | Hyper-methylated | PCa\|NT DMRs |
| chr1 | 32788165 | 32788585 | 0.005968871 | −49.35238095 | Hypo-methylated | PCa\|NT DMRs |
| chr1 | 33390500 | 33391874 | 1.36E−05 | 35.76742857 | Hyper-methylated | PCa\|NT DMRs |
| chr1 | 33928344 | 33938451 | 6.59E−10 | 32.87727891 | Hyper-methylated | PCa\|NT DMRs |
| chr1 | 34628316 | 34628578 | 0.000377317 | 26.22916667 | Hyper-methylated | PCa\|NT DMRs |
| chr1 | 34660621 | 34834336 | 1.17E−06 | −25.03770914 | Hypo-methylated | PCa\|NT DMRs |
| chr1 | 34913158 | 34973225 | 7.31E−05 | −35.01156463 | Hypo-methylated | PCa\|NT DMRs |
| chr1 | 35116130 | 35116544 | 3.66E−05 | −29.70047619 | Hypo-methylated | PCa\|NT DMRs |
| chr1 | 35395855 | 35396461 | 0.002111644 | 21.06043956 | Hyper-methylated | PCa\|NT DMRs |
| chr1 | 36028849 | 36029016 | 0.005968871 | 50.6 | Hyper-methylated | PCa\|NT DMRs |
| chr1 | 36563353 | 36563519 | 0.000176046 | −23.07792208 | Hypo-methylated | PCa\|NT DMRs |
| chr1 | 36643764 | 36644433 | 4.29E−11 | −27.20013605 | Hypo-methylated | PCa\|NT DMRs |
| chr1 | 36832075 | 36832397 | 0.001969641 | 47.33730159 | Hyper-methylated | PCa\|NT DMRs |
| chr1 | 36866393 | 36866855 | 0.003448944 | 28.63265306 | Hyper-methylated | PCa\|NT DMRs |
| chr1 | 36948313 | 36952573 | 6.90E−05 | 33.88015873 | Hyper-methylated | PCa\|NT DMRs |
| chr1 | 36977709 | 37027135 | 2.06E−07 | −23.58852259 | Hypo-methylated | PCa\|NT DMRs |
| chr1 | 37055832 | 37177594 | 6.88E−11 | −27.80623742 | Hypo-methylated | PCa\|NT DMRs |
| chr1 | 37195966 | 37196023 | 0.003622801 | 20.76190476 | Hyper-methylated | PCa\|NT DMRs |
| chr1 | 37204419 | 37240015 | 1.57E−05 | −24.03588435 | Hypo-methylated | PCa\|NT DMRs |
| chr1 | 37266622 | 37307972 | 1.27E−08 | −31.32891156 | Hypo-methylated | PCa\|NT DMRs |
| chr1 | 37333487 | 37351798 | 8.79E−06 | −30.71325052 | Hypo-methylated | PCa\|NT DMRs |
| chr1 | 37357777 | 37447866 | 1.00E−06 | −32.3287619 | Hypo-methylated | PCa\|NT DMRs |
| chr1 | 37448535 | 37476265 | 9.00E−07 | −31.45349206 | Hypo-methylated | PCa\|NT DMRs |
| chr1 | 37549295 | 37561037 | 8.31E−07 | −31.76825397 | Hypo-methylated | PCa\|NT DMRs |
| chr1 | 37579229 | 37645899 | 6.68E−07 | −29.70617284 | Hypo-methylated | PCa\|NT DMRs |
| chr1 | 37735318 | 37756489 | 0.004149771 | −27.89304029 | Hypo-methylated | PCa\|NT DMRs |
| chr1 | 38192490 | 38201159 | 3.09E−06 | 33.65465839 | Hyper-methylated | PCa\|NT DMRs |
| chr1 | 38679372 | 38679582 | 0.005968871 | 34.05714286 | Hyper-methylated | PCa\|NT DMRs |
| chr1 | 38757788 | 38758397 | 6.80E−06 | 23.14264069 | Hyper-methylated | PCa\|NT DMRs |
| chr1 | 39025055 | 39025301 | 1.41E−05 | 36.3085034 | Hyper-methylated | PCa\|NT DMRs |
| chr1 | 39090142 | 39152455 | 9.84E−05 | −21.01117216 | Hypo-methylated | PCa\|NT DMRs |
| chr1 | 39281980 | 39283694 | 1.77E−08 | 34.92922824 | Hyper-methylated | PCa\|NT DMRs |
| chr1 | 39407394 | 39407770 | 0.000481535 | −56.42857143 | Hypo-methylated | PCa\|NT DMRs |
| chr1 | 39546640 | 39546835 | 2.75E−05 | 26.16772487 | Hyper-methylated | PCa\|NT DMRs |
| chr1 | 39957733 | 39958610 | 0.000468139 | 23.11904762 | Hyper-methylated | PCa\|NT DMRs |
| chr1 | 39974878 | 39976077 | 0.003056323 | 33.98148148 | Hyper-methylated | PCa\|NT DMRs |
| chr1 | 40075380 | 40075810 | 0.000241095 | 28.01503759 | Hyper-methylated | PCa\|NT DMRs |
| chr1 | 40137907 | 40149874 | 3.62E−06 | 24.5981417 | Hyper-methylated | PCa\|NT DMRs |
| chr1 | 40253709 | 40253793 | 1.67E−05 | 36.08055556 | Hyper-methylated | PCa\|NT DMRs |
| chr1 | 40268333 | 40269945 | 0.001969641 | 53.58174603 | Hyper-methylated | PCa\|NT DMRs |
| chr1 | 40780274 | 40782303 | 3.73E−11 | 27.26190476 | Hyper-methylated | PCa\|NT DMRs |
| chr1 | 41283622 | 41284920 | 6.78E−07 | 34.73370927 | Hyper-methylated | PCa\|NT DMRs |
| chr1 | 41898163 | 41898446 | 0.000205174 | −51.39285714 | Hypo-methylated | PCa\|NT DMRs |
| chr1 | 41901398 | 41901695 | 0.00068166 | 24.84583333 | Hyper-methylated | PCa\|NT DMRs |
| chr1 | 41947940 | 41948219 | 0.000481535 | −37.83862434 | Hypo-methylated | PCa\|NT DMRs |
| chr1 | 42192789 | 42207041 | 2.20E−06 | 38.9962963 | Hyper-methylated | PCa\|NT DMRs |
| chr1 | 42385901 | 42386064 | 0.005968871 | 38.34285714 | Hyper-methylated | PCa\|NT DMRs |
| chr1 | 42611494 | 42611607 | 3.05E−06 | 27.91208791 | Hyper-methylated | PCa\|NT DMRs |
| chr1 | 43250855 | 43251202 | 3.25E−10 | 44.5270936 | Hyper-methylated | PCa\|NT DMRs |
| chr1 | 43504955 | 43505044 | 0.00064571 | −49.35714286 | Hypo-methylated | PCa\|NT DMRs |
| chr1 | 43701705 | 43701956 | 0.005968871 | −49.57333333 | Hypo-methylated | PCa\|NT DMRs |
| chr1 | 43751201 | 43751567 | 0.00064571 | −40.89795918 | Hypo-methylated | PCa\|NT DMRs |
| chr1 | 43807789 | 43814894 | 1.19E−11 | −46.31965552 | Hypo-methylated | PCa\|NT DMRs |
| chr1 | 43832025 | 43832327 | 0.000974795 | −40.16785714 | Hypo-methylated | PCa\|NT DMRs |
| chr1 | 44031123 | 44031886 | 4.41E−16 | 38.34957599 | Hyper-methylated | PCa\|NT DMRs |
| chr1 | 44070591 | 44070757 | 0.005968871 | −42.82857143 | Hypo-methylated | PCa\|NT DMRs |
| chr1 | 44688915 | 44704214 | 0.00013079 | 36.73483709 | Hyper-methylated | PCa\|NT DMRs |
| chr1 | 44872844 | 44883781 | 1.01E−40 | 25.70754778 | Hyper-methylated | PCa\|NT DMRs |
| chr1 | 45126049 | 45126289 | 0.000215808 | −38.36984127 | Hypo-methylated | PCa\|NT DMRs |
| chr1 | 45267318 | 45267589 | 0.000481535 | 29.74603175 | Hyper-methylated | PCa\|NT DMRs |
| chr1 | 45285916 | 45286086 | 1.42E−05 | 40.87755102 | Hyper-methylated | PCa\|NT DMRs |
| chr1 | 46770707 | 46771105 | 0.000836132 | −25.76719577 | Hypo-methylated | PCa\|NT DMRs |
| chr1 | 46950611 | 46954881 | 1.62E−09 | 23.34227642 | Hyper-methylated | PCa\|NT DMRs |
| chr1 | 48246486 | 48247190 | 1.97E−05 | −31.61666667 | Hypo-methylated | PCa\|NT DMRs |
| chr1 | 48353253 | 48353639 | 0.00068166 | 39.3077381 | Hyper-methylated | PCa\|NT DMRs |
| chr1 | 48462240 | 48462616 | 7.74E−05 | 30.82783883 | Hyper-methylated | PCa\|NT DMRs |
| chr1 | 48555659 | 48564977 | 0.00641359 | −40.68095238 | Hypo-methylated | PCa\|NT DMRs |
| chr1 | 49038737 | 49038934 | 0.005968871 | −31.4952381 | Hypo-methylated | PCa\|NT DMRs |
| chr1 | 50893272 | 50898796 | 1.81E−07 | 21.31452381 | Hyper-methylated | PCa\|NT DMRs |
| chr1 | 51498730 | 51524743 | 0.002767976 | −38.76406926 | Hypo-methylated | PCa\|NT DMRs |
| chr1 | 51795443 | 51795600 | 0.005968871 | −49.25714286 | Hypo-methylated | PCa\|NT DMRs |
| chr1 | 52194940 | 52195821 | 6.68E−21 | 24.93141321 | Hyper-methylated | PCa\|NT DMRs |
| chr1 | 53067881 | 53068491 | 2.16E−33 | 25.51680672 | Hyper-methylated | PCa\|NT DMRs |
| chr1 | 53184149 | 53187506 | 0.00068166 | −22.06845238 | Hypo-methylated | PCa\|NT DMRs |
| chr1 | 53308824 | 53309451 | 2.83E−09 | 21.30588235 | Hyper-methylated | PCa\|NT DMRs |
| chr1 | 53858321 | 53866663 | 1.20E−07 | −22.76870748 | Hypo-methylated | PCa\|NT DMRs |
| chr1 | 53925973 | 53926473 | 8.63E−05 | −23.81873016 | Hypo-methylated | PCa\|NT DMRs |
| chr1 | 53966544 | 53969330 | 0.000632596 | −29.26041667 | Hypo-methylated | PCa\|NT DMRs |
| chr1 | 53992290 | 53994306 | 1.66E−05 | −50.09803922 | Hypo-methylated | PCa\|NT DMRs |
| chr1 | 54040624 | 54041031 | 0.00064571 | 48.9829932 | Hyper-methylated | PCa\|NT DMRs |
| chr1 | 54868836 | 54869222 | 0.003809353 | −24.70380952 | Hypo-methylated | PCa\|NT DMRs |
| chr1 | 54940393 | 54944221 | 3.09E−09 | 25.03543956 | Hyper-methylated | PCa\|NT DMRs |
| chr1 | 54951712 | 54953651 | 9.63E−06 | 25.95646259 | Hyper-methylated | PCa\|NT DMRs |
| chr1 | 54965564 | 54965767 | 0.000974795 | 20.90297619 | Hyper-methylated | PCa\|NT DMRs |
| chr1 | 54979765 | 54980174 | 5.28E−05 | −28.51465201 | Hypo-methylated | PCa\|NT DMRs |
| chr1 | 55008127 | 55008722 | 0.00025701 | 25.99285714 | Hyper-methylated | PCa\|NT DMRs |
| chr1 | 55481436 | 55487895 | 0.005968871 | −27.59077381 | Hypo-methylated | PCa\|NT DMRs |
| chr1 | 55513794 | 55516110 | 0.005513661 | −35.95646259 | Hypo-methylated | PCa\|NT DMRs |
| chr1 | 57285156 | 57285279 | 0.005968871 | −45.05619048 | Hypo-methylated | PCa\|NT DMRs |
| chr1 | 58715050 | 58716179 | 1.18E−20 | 24.64002761 | Hyper-methylated | PCa\|NT DMRs |
| chr1 | 59280256 | 59280466 | 6.67E−10 | −31.71683673 | Hypo-methylated | PCa\|NT DMRs |
| chr1 | 59314696 | 59315051 | 0.001969641 | −34.42063492 | Hypo-methylated | PCa\|NT DMRs |
| chr1 | 61509159 | 61509282 | 8.73E−07 | 23.39855072 | Hyper-methylated | PCa\|NT DMRs |
| chr1 | 61608446 | 61649974 | 0.005513661 | −51.59285714 | Hypo-methylated | PCa\|NT DMRs |
| chr1 | 62793453 | 62793591 | 0.00064571 | −39.69387755 | Hypo-methylated | PCa\|NT DMRs |
| chr1 | 63234270 | 63234475 | 0.005968871 | −57.48571429 | Hypo-methylated | PCa\|NT DMRs |
| chr1 | 64731354 | 64784318 | 0.000176046 | −37.07705628 | Hypo-methylated | PCa\|NT DMRs |
| chr1 | 68038066 | 68048485 | 0.00179283 | −33.51071429 | Hypo-methylated | PCa\|NT DMRs |
| chr1 | 68759638 | 68759826 | 0.005968871 | 22.25714286 | Hyper-methylated | PCa\|NT DMRs |
| chr1 | 72542872 | 72697809 | 3.50E−05 | −31.5970696 | Hypo-methylated | PCa\|NT DMRs |
| chr1 | 73092518 | 73115350 | 6.22E−05 | −21.78877551 | Hypo-methylated | PCa\|NT DMRs |
| chr1 | 73764404 | 74251998 | 2.59E−08 | −29.52310406 | Hypo-methylated | PCa\|NT DMRs |
| chr1 | 74311953 | 74663681 | 0.00284639 | −24.21942857 | Hypo-methylated | PCa\|NT DMRs |
| chr1 | 74754290 | 75159093 | 7.08E−13 | −34.67160827 | Hypo-methylated | PCa\|NT DMRs |
| chr1 | 75297758 | 75579832 | 4.90E−08 | −34.45238095 | Hypo-methylated | PCa\|NT DMRs |
| chr1 | 75616628 | 75687737 | 0.001789867 | −25.23439153 | Hypo-methylated | PCa\|NT DMRs |
| chr1 | 76354705 | 76537546 | 0.002585277 | −34.8625 | Hypo-methylated | PCa\|NT DMRs |
| chr1 | 76540513 | 76540541 | 0.008482405 | 26.74285714 | Hyper-methylated | PCa\|NT DMRs |
| chr1 | 77230619 | 77230837 | 0.000215808 | 23.01587302 | Hyper-methylated | PCa\|NT DMRs |
| chr1 | 77334133 | 77335031 | 1.33E−10 | 33.43320106 | Hyper-methylated | PCa\|NT DMRs |
| chr1 | 77748603 | 77748806 | 0.00064571 | 23.68707483 | Hyper-methylated | PCa\|NT DMRs |
| chr1 | 78140160 | 78147476 | 0.005685503 | −21.96428571 | Hypo-methylated | PCa\|NT DMRs |
| chr1 | 78153343 | 78153404 | 0.001969641 | 26.26190476 | Hyper-methylated | PCa\|NT DMRs |
| chr1 | 78442913 | 78443067 | 0.001969641 | −37.66666667 | Hypo-methylated | PCa\|NT DMRs |
| chr1 | 79982865 | 79982913 | 0.005968871 | −41.28571429 | Hypo-methylated | PCa\|NT DMRs |
| chr1 | 81674230 | 81674454 | 0.004095673 | 49.14285714 | Hyper-methylated | PCa\|NT DMRs |
| chr1 | 81990947 | 81991072 | 0.005968871 | −43.51428571 | Hypo-methylated | PCa\|NT DMRs |
| chr1 | 85463314 | 85463954 | 9.14E−09 | 23.12169312 | Hyper-methylated | PCa\|NT DMRs |
| chr1 | 87598003 | 87598464 | 1.21E−08 | 34.14327731 | Hyper-methylated | PCa\|NT DMRs |
| chr1 | 87678973 | 87767837 | 8.48E−11 | −31.62565284 | Hypo-methylated | PCa\|NT DMRs |
| chr1 | 90309308 | 90309965 | 2.69E−05 | 37.08067227 | Hyper-methylated | PCa\|NT DMRs |
| chr1 | 91692266 | 91703074 | 0.001667552 | −38.41798942 | Hypo-methylated | PCa\|NT DMRs |
| chr1 | 92690657 | 92695075 | 0.000205174 | −29.90654762 | Hypo-methylated | PCa\|NT DMRs |
| chr1 | 93251203 | 93251698 | 0.008581782 | −29.2952381 | Hypo-methylated | PCa\|NT DMRs |
| chr1 | 94146663 | 94146876 | 3.01E−07 | 31.35285714 | Hyper-methylated | PCa\|NT DMRs |
| chr1 | 94159729 | 94206975 | 0.000157376 | −21.36895238 | Hypo-methylated | PCa\|NT DMRs |
| chr1 | 95391231 | 95391315 | 0.000481535 | 25.50793651 | Hyper-methylated | PCa\|NT DMRs |
| chr1 | 95392642 | 95393462 | 1.47E−15 | 29.28767007 | Hyper-methylated | PCa\|NT DMRs |
| chr1 | 96421395 | 96616472 | 9.89E−08 | −30.47505669 | Hypo-methylated | PCa\|NT DMRs |
| chr1 | 98352882 | 98371134 | 0.000974795 | −27.55059524 | Hypo-methylated | PCa\|NT DMRs |
| chr1 | 99006863 | 99089943 | 0.000974795 | −41.83988095 | Hypo-methylated | PCa\|NT DMRs |
| chr1 | 99247365 | 99439568 | 4.33E−05 | −37.05164835 | Hypo-methylated | PCa\|NT DMRs |
| chr1 | 99478270 | 99544615 | 5.79E−09 | −35.16190476 | Hypo-methylated | PCa\|NT DMRs |
| chr1 | 102694337 | 103042273 | 1.23E−05 | −32.48385093 | Hypo-methylated | PCa\|NT DMRs |
| chr1 | 104434280 | 104616514 | 1.64E−13 | −34.23446712 | Hypo-methylated | PCa\|NT DMRs |
| chr1 | 104695970 | 105142681 | 1.96E−22 | −31.12258503 | Hypo-methylated | PCa\|NT DMRs |
| chr1 | 108023308 | 108023483 | 0.000974795 | 39.51785714 | Hyper-methylated | PCa\|NT DMRs |
| chr1 | 109655734 | 109655971 | 0.000570576 | 26.84391534 | Hyper-methylated | PCa\|NT DMRs |
| chr1 | 109783324 | 109783651 | 0.002534928 | −26.1 | Hypo-methylated | PCa\|NT DMRs |
| chr1 | 109806825 | 109806896 | 0.000205174 | −37.97321429 | Hypo-methylated | PCa\|NT DMRs |
| chr1 | 110009924 | 110010025 | 0.000861954 | 27.30753968 | Hyper-methylated | PCa\|NT DMRs |
| chr1 | 110086450 | 110086987 | 0.00355142 | −24.56776557 | Hypo-methylated | PCa\|NT DMRs |
| chr1 | 110166638 | 110167153 | 3.61E−05 | 37.19795918 | Hyper-methylated | PCa\|NT DMRs |
| chr1 | 110254680 | 110254776 | 0.001076343 | 30.45887446 | Hyper-methylated | PCa\|NT DMRs |
| chr1 | 110395903 | 110396099 | 0.004057568 | 33.1984127 | Hyper-methylated | PCa\|NT DMRs |
| chr1 | 110672715 | 110673214 | 8.23E−08 | 34.18024691 | Hyper-methylated | PCa\|NT DMRs |
| chr1 | 110754054 | 110754872 | 2.89E−13 | 24.75137255 | Hyper-methylated | PCa\|NT DMRs |
| chr1 | 112016297 | 112016636 | 0.000822388 | 44.18285714 | Hyper-methylated | PCa\|NT DMRs |
| chr1 | 112058195 | 112058444 | 9.30E−06 | 30.83035714 | Hyper-methylated | PCa\|NT DMRs |
| chr1 | 112504962 | 112508570 | 0.007679542 | 31.36349206 | Hyper-methylated | PCa\|NT DMRs |
| chr1 | 112933091 | 112933235 | 0.00064571 | −40.40816327 | Hypo-methylated | PCa\|NT DMRs |
| chr1 | 113181278 | 113184418 | 0.000836132 | −28.28835979 | Hypo-methylated | PCa\|NT DMRs |
| chr1 | 113265661 | 113265809 | 4.90E−08 | 44.11580087 | Hyper-methylated | PCa\|NT DMRs |
| chr1 | 113397255 | 113397302 | 0.005968871 | 25.87904762 | Hyper-methylated | PCa\|NT DMRs |
| chr1 | 115231628 | 115238458 | 0.001241245 | −23.48412698 | Hypo-methylated | PCa\|NT DMRs |
| chr1 | 115333233 | 115333495 | 0.000547122 | −23.1697479 | Hypo-methylated | PCa\|NT DMRs |
| chr1 | 115880730 | 115881506 | 1.95E−05 | 26.65958188 | Hyper-methylated | PCa\|NT DMRs |
| chr1 | 116004434 | 116088091 | 0.000261152 | −27.06819728 | Hypo-methylated | PCa\|NT DMRs |
| chr1 | 116666737 | 116666937 | 0.005968871 | −57.54285714 | Hypo-methylated | PCa\|NT DMRs |
| chr1 | 116710405 | 116770527 | 8.97E−09 | 34.10348432 | Hyper-methylated | PCa\|NT DMRs |
| chr1 | 116918436 | 116918833 | 0.00064571 | −39.19727891 | Hypo-methylated | PCa\|NT DMRs |
| chr1 | 117156409 | 117156642 | 2.86E−05 | −26.31135531 | Hypo-methylated | PCa\|NT DMRs |
| chr1 | 117283501 | 117299914 | 0.005968871 | 25.24761905 | Hyper-methylated | PCa\|NT DMRs |
| chr1 | 118114942 | 118115242 | 0.005513661 | −36.26964286 | Hypo-methylated | PCa\|NT DMRs |
| chr1 | 119870029 | 119870803 | 3.56E−08 | 38.3638448 | Hyper-methylated | PCa\|NT DMRs |
| chr1 | 120166206 | 120168799 | 0.000190021 | −37.7952381 | Hypo-methylated | PCa\|NT DMRs |
| chr1 | 120436834 | 120437114 | 0.002335477 | 28.12244898 | Hyper-methylated | PCa\|NT DMRs |
| chr1 | 120906320 | 120906355 | 0.005968871 | −55.17142857 | Hypo-methylated | PCa\|NT DMRs |
| chr1 | 143663862 | 143664368 | 3.66E−05 | 25.75809524 | Hyper-methylated | PCa\|NT DMRs |
| chr1 | 147295926 | 147301218 | 0.001969641 | −22.42857143 | Hypo-methylated | PCa\|NT DMRs |
| chr1 | 147718179 | 147718760 | 0.004553481 | 26.62539683 | Hyper-methylated | PCa\|NT DMRs |
| chr1 | 147825907 | 147826178 | 3.87E−05 | 30.8977591 | Hyper-methylated | PCa\|NT DMRs |
| chr1 | 148891082 | 148897613 | 0.00046742 | −35.35714286 | Hypo-methylated | PCa\|NT DMRs |
| chr1 | 149162565 | 149180525 | 0.001181579 | −23.4010989 | Hypo-methylated | PCa\|NT DMRs |
| chr1 | 149197879 | 149222789 | 0.000143323 | −24.5475 | Hypo-methylated | PCa\|NT DMRs |
| chr1 | 149231293 | 149284147 | 0.001758998 | −22.49174603 | Hypo-methylated | PCa\|NT DMRs |
| chr1 | 149672605 | 149672888 | 0.001969641 | 45.26190476 | Hyper-methylated | PCa\|NT DMRs |
| chr1 | 149712217 | 149719537 | 2.85E−06 | 29.41493506 | Hyper-methylated | PCa\|NT DMRs |
| chr1 | 150134720 | 150135088 | 0.005968871 | 44.14285714 | Hyper-methylated | PCa\|NT DMRs |
| chr1 | 150516925 | 150519680 | 0.004095673 | −39.56349206 | Hypo-methylated | PCa\|NT DMRs |
| chr1 | 150523439 | 150525535 | 0.004178506 | 25.53690476 | Hyper-methylated | PCa\|NT DMRs |
| chr1 | 150897012 | 150898078 | 0.005243408 | 23.54652015 | Hyper-methylated | PCa\|NT DMRs |
| chr1 | 150947666 | 150951875 | 1.75E−05 | 28.9225 | Hyper-methylated | PCa\|NT DMRs |
| chr1 | 151482593 | 151483096 | 0.000556452 | −29.06972789 | Hypo-methylated | PCa\|NT DMRs |
| chr1 | 151675661 | 151676614 | 0.001216085 | 26.21148459 | Hyper-methylated | PCa\|NT DMRs |
| chr1 | 151773258 | 151773306 | 0.005968871 | 36.17142857 | Hyper-methylated | PCa\|NT DMRs |
| chr1 | 152051042 | 152073000 | 7.14E−06 | −23.12014652 | Hypo-methylated | PCa\|NT DMRs |
| chr1 | 152506528 | 153195799 | 1.31E−14 | −31.33899749 | Hypo-methylated | PCa\|NT DMRs |
| chr1 | 153312923 | 153327231 | 2.29E−06 | −32.85661376 | Hypo-methylated | PCa\|NT DMRs |
| chr1 | 153670233 | 153670846 | 8.31E−07 | 41.16517857 | Hyper-methylated | PCa\|NT DMRs |
| chr1 | 153761958 | 153762187 | 0.003809353 | −23.62619048 | Hypo-methylated | PCa\|NT DMRs |
| chr1 | 153762201 | 153762518 | 0.001181579 | 26.14285714 | Hyper-methylated | PCa\|NT DMRs |
| chr1 | 154166483 | 154172333 | 0.007560755 | −33.66785714 | Hypo-methylated | PCa\|NT DMRs |
| chr1 | 154475135 | 154475936 | 3.45E−05 | 20.95887446 | Hyper-methylated | PCa\|NT DMRs |
| chr1 | 154681606 | 154685576 | 0.000377317 | −21.08035714 | Hypo-methylated | PCa\|NT DMRs |
| chr1 | 154786156 | 154786338 | 0.005968871 | −46.28571429 | Hypo-methylated | PCa\|NT DMRs |
| chr1 | 154808682 | 154811115 | 0.003464153 | −28.48484848 | Hypo-methylated | PCa\|NT DMRs |
| chr1 | 154843162 | 154851481 | 0.005618668 | −23.1026455 | Hypo-methylated | PCa\|NT DMRs |
| chr1 | 154971110 | 154971872 | 0.000117699 | 30.00865801 | Hyper-methylated | PCa\|NT DMRs |
| chr1 | 155097713 | 155098952 | 1.15E−07 | −33.830839 | Hypo-methylated | PCa\|NT DMRs |
| chr1 | 155220243 | 155224084 | 1.43E−06 | 36.72380952 | Hyper-methylated | PCa\|NT DMRs |
| chr1 | 155264329 | 155265468 | 1.72E−09 | 22.63065057 | Hyper-methylated | PCa\|NT DMRs |
| chr1 | 155290759 | 155291206 | 0.002994094 | 31.995671 | Hyper-methylated | PCa\|NT DMRs |
| chr1 | 155892560 | 155903223 | 0.006225823 | −34.0004329 | Hypo-methylated | PCa\|NT DMRs |
| chr1 | 156046344 | 156046864 | 2.49E−12 | 40.87116402 | Hyper-methylated | PCa\|NT DMRs |
| chr1 | 156051240 | 156051524 | 8.90E−07 | 26.91428571 | Hyper-methylated | PCa\|NT DMRs |
| chr1 | 156211434 | 156211494 | 0.001115302 | 25.87857143 | Hyper-methylated | PCa\|NT DMRs |
| chr1 | 156255142 | 156256264 | 7.92E−06 | −41.5837535 | Hypo-methylated | PCa\|NT DMRs |
| chr1 | 156405371 | 156405689 | 1.50E−08 | 29.13238095 | Hyper-methylated | PCa\|NT DMRs |
| chr1 | 156661102 | 156662023 | 0.000609882 | 37.06349206 | Hyper-methylated | PCa\|NT DMRs |
| chr1 | 156713538 | 156715050 | 3.05E−06 | 26.82857143 | Hyper-methylated | PCa\|NT DMRs |
| chr1 | 156785879 | 156786689 | 0.009807302 | 27.53142857 | Hyper-methylated | PCa\|NT DMRs |
| chr1 | 156813949 | 156821140 | 3.06E−05 | 33.23579639 | Hyper-methylated | PCa\|NT DMRs |
| chr1 | 157258369 | 157346444 | 9.43E−05 | −25.6996337 | Hypo-methylated | PCa\|NT DMRs |
| chr1 | 158083971 | 158085900 | 0.000132887 | −35.50952381 | Hypo-methylated | PCa\|NT DMRs |
| chr1 | 158098549 | 158118900 | 0.002097193 | −37.64285714 | Hypo-methylated | PCa\|NT DMRs |
| chr1 | 160347295 | 160365196 | 0.000215808 | −22.61428571 | Hypo-methylated | PCa\|NT DMRs |
| chr1 | 160376813 | 160582932 | 5.60E−12 | −28.73435374 | Hypo-methylated | PCa\|NT DMRs |
| chr1 | 160751831 | 160760529 | 0.004659837 | −28.05952381 | Hypo-methylated | PCa\|NT DMRs |
| chr1 | 161035517 | 161039594 | 0.000145171 | 54.42278912 | Hyper-methylated | PCa\|NT DMRs |
| chr1 | 161103733 | 161104036 | 0.009106413 | −37.47619048 | Hypo-methylated | PCa\|NT DMRs |
| chr1 | 161167870 | 161168394 | 1.71E−05 | −31.74461153 | Hypo-methylated | PCa\|NT DMRs |
| chr1 | 161585585 | 161586729 | 1.87E−05 | −22.07936508 | Hypo-methylated | PCa\|NT DMRs |
| chr1 | 161591532 | 161592118 | 3.11E−05 | 21.7010582 | Hyper-methylated | PCa\|NT DMRs |
| chr1 | 164669296 | 164681798 | 0.000196301 | 33.69920635 | Hyper-methylated | PCa\|NT DMRs |
| chr1 | 165325913 | 165326314 | 3.10E−05 | 29.27731092 | Hyper-methylated | PCa\|NT DMRs |
| chr1 | 165913078 | 166002328 | 2.86E−07 | −40 | Hypo-methylated | PCa\|NT DMRs |
| chr1 | 166853374 | 166853813 | 5.25E−10 | 25.49768707 | Hyper-methylated | PCa\|NT DMRs |
| chr1 | 166998500 | 166999070 | 0.000136862 | −24.92698413 | Hypo-methylated | PCa\|NT DMRs |
| chr1 | 167094887 | 167095593 | 0.001969641 | −52.65 | Hypo-methylated | PCa\|NT DMRs |
| chr1 | 167498532 | 167498592 | 0.005968871 | −35.71428571 | Hypo-methylated | PCa\|NT DMRs |
| chr1 | 167572289 | 167572355 | 0.008482405 | 56.51428571 | Hyper-methylated | PCa\|NT DMRs |
| chr1 | 168302983 | 168399471 | 0.001241245 | −33.53703704 | Hypo-methylated | PCa\|NT DMRs |
| chr1 | 170082030 | 170500398 | 3.79E−08 | −32.20819491 | Hypo-methylated | PCa\|NT DMRs |
| chr1 | 170749435 | 170826681 | 0.000114755 | −31.91941392 | Hypo-methylated | PCa\|NT DMRs |
| chr1 | 173854852 | 173854985 | 0.001969641 | 44.11904762 | Hyper-methylated | PCa\|NT DMRs |
| chr1 | 175037023 | 175048874 | 2.05E−13 | −23.65551684 | Hypo-methylated | PCa\|NT DMRs |
| chr1 | 175338190 | 175463492 | 1.07E−13 | −23.52293763 | Hypo-methylated | PCa\|NT DMRs |
| chr1 | 178482861 | 178483003 | 0.001969641 | −55.57142857 | Hypo-methylated | PCa\|NT DMRs |
| chr1 | 179264999 | 179265029 | 0.008482405 | −29.96666667 | Hypo-methylated | PCa\|NT DMRs |
| chr1 | 179334235 | 179334537 | 0.003391914 | −33.88941799 | Hypo-methylated | PCa\|NT DMRs |
| chr1 | 179922654 | 179922703 | 0.005968871 | 34.05619048 | Hyper-methylated | PCa\|NT DMRs |
| chr1 | 180176488 | 180182756 | 0.003892841 | 32.19047619 | Hyper-methylated | PCa\|NT DMRs |
| chr1 | 180202396 | 180203116 | 3.00E−19 | 20.76048237 | Hyper-methylated | PCa\|NT DMRs |
| chr1 | 180882232 | 180882453 | 5.75E−09 | 26.85714286 | Hyper-methylated | PCa\|NT DMRs |
| chr1 | 181073954 | 181074190 | 9.10E−08 | 23.17573696 | Hyper-methylated | PCa\|NT DMRs |
| chr1 | 181097831 | 181101599 | 0.004149771 | 34.07032967 | Hyper-methylated | PCa\|NT DMRs |
| chr1 | 181803019 | 181944036 | 0.001969641 | −36.78730159 | Hypo-methylated | PCa\|NT DMRs |
| chr1 | 182025773 | 182026562 | 7.84E−08 | 24.21269841 | Hyper-methylated | PCa\|NT DMRs |
| chr1 | 182310800 | 182316426 | 0.001969641 | −36.71825397 | Hypo-methylated | PCa\|NT DMRs |
| chr1 | 183386275 | 183386606 | 3.87E−08 | 26.57769424 | Hyper-methylated | PCa\|NT DMRs |
| chr1 | 184005493 | 184006015 | 4.11E−07 | 25.9037594 | Hyper-methylated | PCa\|NT DMRs |
| chr1 | 184618881 | 184639635 | 0.004711566 | 33.57983193 | Hyper-methylated | PCa\|NT DMRs |
| chr1 | 185663114 | 185663347 | 0.002097193 | 31.88435374 | Hyper-methylated | PCa\|NT DMRs |
| chr1 | 185721185 | 186230991 | 5.84E−05 | −47.35039683 | Hypo-methylated | PCa\|NT DMRs |
| chr1 | 186649692 | 186649986 | 5.83E−05 | 33.42857143 | Hyper-methylated | PCa\|NT DMRs |
| chr1 | 189063671 | 189109551 | 2.24E−08 | −22.41005291 | Hypo-methylated | PCa\|NT DMRs |
| chr1 | 189469044 | 189509725 | 0.000783791 | −24.51857143 | Hypo-methylated | PCa\|NT DMRs |
| chr1 | 190761371 | 190994247 | 6.41E−10 | −35.75697865 | Hypo-methylated | PCa\|NT DMRs |
| chr1 | 193253015 | 193253150 | 0.005968871 | −37.67428571 | Hypo-methylated | PCa\|NT DMRs |
| chr1 | 194312007 | 195888354 | 1.83E−50 | −34.29199485 | Hypo-methylated | PCa\|NT DMRs |
| chr1 | 196115710 | 196537618 | 4.50E−08 | −35.59504762 | Hypo-methylated | PCa\|NT DMRs |
| chr1 | 196611329 | 196844068 | 5.19E−07 | −38.29 | Hypo-methylated | PCa\|NT DMRs |
| chr1 | 196932515 | 197038359 | 1.67E−07 | −29.35119048 | Hypo-methylated | PCa\|NT DMRs |
| chr1 | 197304856 | 197324767 | 1.76E−06 | −32.02380952 | Hypo-methylated | PCa\|NT DMRs |
| chr1 | 197946957 | 197963991 | 0.000205174 | −38.80357143 | Hypo-methylated | PCa\|NT DMRs |
| chr1 | 200003048 | 200008049 | 2.24E−14 | 30.85496368 | Hyper-methylated | PCa\|NT DMRs |
| chr1 | 200456599 | 200482173 | 7.87E−05 | −40.63053221 | Hypo-methylated | PCa\|NT DMRs |
| chr1 | 200842697 | 200843427 | 1.80E−13 | 32.76537967 | Hyper-methylated | PCa\|NT DMRs |
| chr1 | 201154172 | 201175371 | 0.0078067 | −23.11764706 | Hypo-methylated | PCa\|NT DMRs |
| chr1 | 201228945 | 201229165 | 0.001969641 | 52.64285714 | Hyper-methylated | PCa\|NT DMRs |
| chr1 | 201244318 | 201244536 | 0.000957078 | −27.72727273 | Hypo-methylated | PCa\|NT DMRs |
| chr1 | 201252201 | 201253432 | 1.99E−11 | 27.24743231 | Hyper-methylated | PCa\|NT DMRs |
| chr1 | 201709358 | 201710008 | 1.30E−10 | 28.24039939 | Hyper-methylated | PCa\|NT DMRs |
| chr1 | 201858339 | 201858839 | 0.00512564 | 22.98280423 | Hyper-methylated | PCa\|NT DMRs |
| chr1 | 202091944 | 202092296 | 0.000205174 | −58 | Hypo-methylated | PCa\|NT DMRs |
| chr1 | 202125897 | 202132435 | 3.02E−07 | 42.0474026 | Hyper-methylated | PCa\|NT DMRs |
| chr1 | 202168301 | 202168650 | 0.00064571 | 40.96598639 | Hyper-methylated | PCa\|NT DMRs |
| chr1 | 202210873 | 202211194 | 0.001969641 | 34.45238095 | Hyper-methylated | PCa\|NT DMRs |
| chr1 | 202614001 | 202614128 | 0.001969641 | 49.1547619 | Hyper-methylated | PCa\|NT DMRs |
| chr1 | 202772807 | 202772872 | 0.005968871 | 35.51428571 | Hyper-methylated | PCa\|NT DMRs |
| chr1 | 203184087 | 203186963 | 0.005513661 | 26.46428571 | Hyper-methylated | PCa\|NT DMRs |
| chr1 | 203242717 | 203243001 | 0.003448944 | 33.66326531 | Hyper-methylated | PCa\|NT DMRs |
| chr1 | 203281669 | 203282013 | 0.005006723 | 25.26428571 | Hyper-methylated | PCa\|NT DMRs |
| chr1 | 203457686 | 203477847 | 0.002562638 | −20.35674603 | Hypo-methylated | PCa\|NT DMRs |
| chr1 | 203595061 | 203598800 | 1.75E−11 | 27.75462963 | Hyper-methylated | PCa\|NT DMRs |
| chr1 | 203695223 | 203731459 | 7.79E−05 | −21.39285714 | Hypo-methylated | PCa\|NT DMRs |
| chr1 | 204056651 | 204073611 | 0.001899053 | −33.63865546 | Hypo-methylated | PCa\|NT DMRs |
| chr1 | 204165654 | 204165825 | 0.002094 | −28.57380952 | Hypo-methylated | PCa\|NT DMRs |
| chr1 | 204300954 | 204301279 | 0.002097193 | −37.32653061 | Hypo-methylated | PCa\|NT DMRs |
| chr1 | 204348059 | 204348448 | 0.005968871 | −56.7047619 | Hypo-methylated | PCa\|NT DMRs |
| chr1 | 204695100 | 204721480 | 0.000709087 | −21.75602241 | Hypo-methylated | PCa\|NT DMRs |
| chr1 | 205039042 | 205039094 | 3.66E−05 | −27.08333333 | Hypo-methylated | PCa\|NT DMRs |
| chr1 | 205226422 | 205226532 | 6.42E−05 | −38.54603175 | Hypo-methylated | PCa\|NT DMRs |
| chr1 | 205312571 | 205313129 | 6.74E−10 | 32.21574344 | Hyper-methylated | PCa\|NT DMRs |
| chr1 | 205424275 | 205424899 | 0.001210062 | 29.0670068 | Hyper-methylated | PCa\|NT DMRs |
| chr1 | 205449598 | 205453924 | 0.003034084 | 24.77380952 | Hyper-methylated | PCa\|NT DMRs |
| chr1 | 205537857 | 205539322 | 2.00E−06 | 21.66488095 | Hyper-methylated | PCa\|NT DMRs |
| chr1 | 206223089 | 206223320 | 0.000696555 | 38.50649351 | Hyper-methylated | PCa\|NT DMRs |
| chr1 | 206644147 | 206644492 | 1.42E−05 | 20.13605442 | Hyper-methylated | PCa\|NT DMRs |
| chr1 | 206679959 | 206680526 | 1.23E−17 | 23.51253133 | Hyper-methylated | PCa\|NT DMRs |
| chr1 | 207198170 | 207201166 | 0.003400006 | −42.67346939 | Hypo-methylated | PCa\|NT DMRs |
| chr1 | 207596832 | 207598015 | 8.35E−05 | −31.01948052 | Hypo-methylated | PCa\|NT DMRs |
| chr1 | 207669481 | 207669836 | 7.86E−09 | 21.45142857 | Hyper-methylated | PCa\|NT DMRs |
| chr1 | 207987203 | 208000382 | 3.44E−06 | −44.11519274 | Hypo-methylated | PCa\|NT DMRs |
| chr1 | 208084005 | 208096210 | 0.006096657 | 29.11428571 | Hyper-methylated | PCa\|NT DMRs |
| chr1 | 208808282 | 209315673 | 2.94E−20 | −28.27251553 | Hypo-methylated | PCa\|NT DMRs |
| chr1 | 209405088 | 209405373 | 1.29E−05 | 24.69705215 | Hyper-methylated | PCa\|NT DMRs |
| chr1 | 209800707 | 209801480 | 0.002400984 | −28.19047619 | Hypo-methylated | PCa\|NT DMRs |
| chr1 | 209974625 | 209979044 | 0.003892841 | −38.46984127 | Hypo-methylated | PCa\|NT DMRs |
| chr1 | 210036434 | 210062618 | 2.54E−09 | −29.24025974 | Hypo-methylated | PCa\|NT DMRs |
| chr1 | 211176208 | 211288577 | 7.60E−08 | −22.29325397 | Hypo-methylated | PCa\|NT DMRs |
| chr1 | 211652352 | 211652728 | 1.97E−05 | 37.07142857 | Hyper-methylated | PCa\|NT DMRs |
| chr1 | 211898416 | 211909182 | 0.001969641 | −23.2047619 | Hypo-methylated | PCa\|NT DMRs |
| chr1 | 212415691 | 212416118 | 0.001115302 | −39.82619048 | Hypo-methylated | PCa\|NT DMRs |
| chr1 | 212927674 | 212927846 | 0.001969641 | 57.67063492 | Hyper-methylated | PCa\|NT DMRs |
| chr1 | 213089938 | 213090272 | 1.09E−06 | 36.46414566 | Hyper-methylated | PCa\|NT DMRs |
| chr1 | 213154446 | 213155055 | 9.66E−07 | −44.3526455 | Hypo-methylated | PCa\|NT DMRs |
| chr1 | 214169817 | 214171382 | 6.42E−05 | −35.42539683 | Hypo-methylated | PCa\|NT DMRs |
| chr1 | 214843371 | 214907951 | 1.75E−06 | −33.02142857 | Hypo-methylated | PCa\|NT DMRs |
| chr1 | 215810695 | 216251674 | 4.48E−06 | −24.88571429 | Hypo-methylated | PCa\|NT DMRs |
| chr1 | 218067939 | 218076279 | 1.42E−05 | −36.50510204 | Hypo-methylated | PCa\|NT DMRs |
| chr1 | 218139870 | 218257352 | 2.57E−06 | −24.09551821 | Hypo-methylated | PCa\|NT DMRs |
| chr1 | 220132345 | 220136931 | 0.001969641 | −53.36734694 | Hypo-methylated | PCa\|NT DMRs |
| chr1 | 220978575 | 220988536 | 0.001610287 | 27.3422619 | Hyper-methylated | PCa\|NT DMRs |
| chr1 | 221060802 | 221067932 | 2.75E−07 | 22.02638353 | Hyper-methylated | PCa\|NT DMRs |
| chr1 | 221067947 | 221291115 | 2.66E−08 | −20.94905869 | Hypo-methylated | PCa\|NT DMRs |
| chr1 | 221482362 | 221509985 | 0.004095673 | −29.1984127 | Hypo-methylated | PCa\|NT DMRs |
| chr1 | 223374069 | 223382516 | 3.36E−06 | −22.4914966 | Hypo-methylated | PCa\|NT DMRs |
| chr1 | 223440701 | 223440923 | 0.001969641 | −46.38095238 | Hypo-methylated | PCa\|NT DMRs |
| chr1 | 223538174 | 223538701 | 1.90E−12 | 27.85933076 | Hyper-methylated | PCa\|NT DMRs |
| chr1 | 225655019 | 225656210 | 2.86E−05 | 55.2967033 | Hyper-methylated | PCa\|NT DMRs |
| chr1 | 225865166 | 225865315 | 2.04E−05 | 44.13781513 | Hyper-methylated | PCa\|NT DMRs |
| chr1 | 226065204 | 226065429 | 0.008482405 | −40.57142857 | Hypo-methylated | PCa\|NT DMRs |
| chr1 | 226310783 | 226323692 | 9.84E−06 | −34.60555556 | Hypo-methylated | PCa\|NT DMRs |
| chr1 | 226537535 | 226538388 | 0.00068166 | 36.91802721 | Hyper-methylated | PCa\|NT DMRs |
| chr1 | 226556933 | 226557092 | 0.005968871 | 37.54285714 | Hyper-methylated | PCa\|NT DMRs |
| chr1 | 226593128 | 226593303 | 0.003622801 | 27.74206349 | Hyper-methylated | PCa\|NT DMRs |
| chr1 | 226899756 | 226900922 | 0.00064571 | 34.62030075 | Hyper-methylated | PCa\|NT DMRs |
| chr1 | 228195101 | 228197370 | 6.49E−05 | 25.99786535 | Hyper-methylated | PCa\|NT DMRs |
| chr1 | 228201122 | 228201256 | 5.84E−05 | −46.53174603 | Hypo-methylated | PCa\|NT DMRs |
| chr1 | 228246580 | 228246882 | 0.001775064 | 21.90436508 | Hyper-methylated | PCa\|NT DMRs |
| chr1 | 228329098 | 228329455 | 0.001969641 | −30.15428571 | Hypo-methylated | PCa\|NT DMRs |
| chr1 | 228344312 | 228345813 | 0.000196301 | 34.47896825 | Hyper-methylated | PCa\|NT DMRs |
| chr1 | 228354439 | 228354576 | 0.000570576 | −24.94505495 | Hypo-methylated | PCa\|NT DMRs |
| chr1 | 228401736 | 228402001 | 5.62E−06 | 25.7473545 | Hyper-methylated | PCa\|NT DMRs |
| chr1 | 228633876 | 228634336 | 3.69E−11 | 28.0552381 | Hyper-methylated | PCa\|NT DMRs |
| chr1 | 228784167 | 228786296 | 5.40E−05 | 34.82352941 | Hyper-methylated | PCa\|NT DMRs |
| chr1 | 228857345 | 228858255 | 0.001969641 | −57.14965986 | Hypo-methylated | PCa\|NT DMRs |
| chr1 | 228890579 | 228891071 | 5.32E−05 | 20.83134921 | Hyper-methylated | PCa\|NT DMRs |
| chr1 | 229033902 | 229057326 | 0.005685126 | 32.25677656 | Hyper-methylated | PCa\|NT DMRs |
| chr1 | 229203546 | 229203784 | 0.000205174 | 39.44642857 | Hyper-methylated | PCa\|NT DMRs |
| chr1 | 229283045 | 229283746 | 5.23E−08 | 32.18653061 | Hyper-methylated | PCa\|NT DMRs |
| chr1 | 229917349 | 229917561 | 0.005968871 | −35.84952381 | Hypo-methylated | PCa\|NT DMRs |
| chr1 | 230193513 | 230193574 | 0.005968871 | 36.53333333 | Hyper-methylated | PCa\|NT DMRs |
| chr1 | 230883707 | 230884198 | 1.11E−05 | −33.0952381 | Hypo-methylated | PCa\|NT DMRs |
| chr1 | 230976177 | 230976487 | 0.000974795 | −28.99702381 | Hypo-methylated | PCa\|NT DMRs |
| chr1 | 231176368 | 231176964 | 5.13E−13 | 22.14545455 | Hyper-methylated | PCa\|NT DMRs |
| chr1 | 231263394 | 231280946 | 0.000117699 | −34.53982684 | Hypo-methylated | PCa\|NT DMRs |
| chr1 | 231296607 | 231297254 | 7.29E−07 | 22.64603175 | Hyper-methylated | PCa\|NT DMRs |
| chr1 | 231299236 | 231299306 | 1.59E−05 | 22.65914787 | Hyper-methylated | PCa\|NT DMRs |
| chr1 | 231347678 | 231347818 | 0.001969641 | −54.22619048 | Hypo-methylated | PCa\|NT DMRs |
| chr1 | 231830537 | 231830815 | 0.001969641 | 43.38492063 | Hyper-methylated | PCa\|NT DMRs |
| chr1 | 232941139 | 232941642 | 2.39E−12 | 32.72522523 | Hyper-methylated | PCa\|NT DMRs |
| chr1 | 234261126 | 234261358 | 6.42E−05 | 33.12169312 | Hyper-methylated | PCa\|NT DMRs |
| chr1 | 234350793 | 234350835 | 0.008482405 | −33.30285714 | Hypo-methylated | PCa\|NT DMRs |
| chr1 | 234398333 | 234398881 | 0.002097193 | −39.66938776 | Hypo-methylated | PCa\|NT DMRs |
| chr1 | 234445221 | 234445412 | 0.004095673 | −52.23809524 | Hypo-methylated | PCa\|NT DMRs |
| chr1 | 234792339 | 234792735 | 0.002585277 | 20.82916667 | Hyper-methylated | PCa\|NT DMRs |
| chr1 | 235105604 | 235105650 | 0.008482405 | −40.02857143 | Hypo-methylated | PCa\|NT DMRs |
| chr1 | 235123103 | 235123738 | 0.008933897 | −40.57482993 | Hypo-methylated | PCa\|NT DMRs |
| chr1 | 235148032 | 235148290 | 0.005968871 | 60.12 | Hyper-methylated | PCa\|NT DMRs |
| chr1 | 235184121 | 235243042 | 0.007959104 | −20.32896825 | Hypo-methylated | PCa\|NT DMRs |
| chr1 | 235255977 | 235256647 | 0.000185577 | −42.72380952 | Hypo-methylated | PCa\|NT DMRs |
| chr1 | 236768539 | 236799430 | 2.89E−06 | −20.47204969 | Hypo-methylated | PCa\|NT DMRs |
| chr1 | 238011524 | 239092153 | 1.25E−36 | −34.62134532 | Hypo-methylated | PCa\|NT DMRs |
| chr1 | 239401388 | 239401479 | 0.000974795 | −34.1547619 | Hypo-methylated | PCa\|NT DMRs |
| chr1 | 240170594 | 240175715 | 0.001181579 | −20.35034014 | Hypo-methylated | PCa\|NT DMRs |
| chr1 | 240256554 | 240257051 | 1.17E−17 | 23.64170772 | Hyper-methylated | PCa\|NT DMRs |
| chr1 | 241541644 | 241575388 | 0.001969641 | −46.97222222 | Hypo-methylated | PCa\|NT DMRs |
| chr1 | 241715148 | 241715903 | 0.004553481 | 41.50529101 | Hyper-methylated | PCa\|NT DMRs |
| chr1 | 241729888 | 241730263 | 0.001969641 | −46.82539683 | Hypo-methylated | PCa\|NT DMRs |
| chr1 | 243074022 | 243255156 | 2.96E−08 | −24.66877289 | Hypo-methylated | PCa\|NT DMRs |
| chr1 | 244243867 | 244244145 | 0.001480644 | −20.02857143 | Hypo-methylated | PCa\|NT DMRs |
| chr1 | 244387932 | 244388473 | 0.008482405 | 47.8152381 | Hyper-methylated | PCa\|NT DMRs |
| chr1 | 245217861 | 245218040 | 0.005968871 | 49.1 | Hyper-methylated | PCa\|NT DMRs |
| chr1 | 245736547 | 245736596 | 0.005968871 | −32.41142857 | Hypo-methylated | PCa\|NT DMRs |
| chr1 | 245772696 | 245773403 | 1.97E−05 | −55.05714286 | Hypo-methylated | PCa\|NT DMRs |
| chr1 | 246866819 | 246867124 | 0.008234951 | 32.37142857 | Hyper-methylated | PCa\|NT DMRs |
| chr1 | 246939151 | 246939711 | 8.35E−08 | −50.01492063 | Hypo-methylated | PCa\|NT DMRs |
| chr1 | 246945661 | 246945871 | 0.005968871 | 43.71428571 | Hyper-methylated | PCa\|NT DMRs |
| chr1 | 247535548 | 247535896 | 0.001969641 | −44.76190476 | Hypo-methylated | PCa\|NT DMRs |
| chr1 | 247538743 | 247573800 | 1.87E−06 | −25.68571429 | Hypo-methylated | PCa\|NT DMRs |
| chr1 | 247590008 | 247614728 | 2.39E−08 | −34.12802198 | Hypo-methylated | PCa\|NT DMRs |
| chr1 | 248143991 | 248481926 | 3.09E−10 | −30.7972973 | Hypo-methylated | PCa\|NT DMRs |
| chr1 | 248685329 | 248685733 | 7.19E−05 | −28.08531746 | Hypo-methylated | PCa\|NT DMRs |
| chr1 | 249090768 | 249097012 | 0.001344447 | −26.875 | Hypo-methylated | PCa\|NT DMRs |
| chr1 | 249097719 | 249099771 | 0.000456684 | 30.74206349 | Hyper-methylated | PCa\|NT DMRs |
| chr2 | 11910 | 14238 | 0.008511532 | −32.38461538 | Hypo-methylated | PCa\|NT DMRs |
| chr2 | 45448 | 46433 | 3.18E−25 | 20.43745645 | Hyper-methylated | PCa\|NT DMRs |
| chr2 | 439970 | 440149 | 0.001969641 | −28.52380952 | Hypo-methylated | PCa\|NT DMRs |
| chr2 | 496069 | 497417 | 1.02E−09 | −26.19708995 | Hypo-methylated | PCa\|NT DMRs |
| chr2 | 636544 | 637016 | 7.46E−11 | 28.13809524 | Hyper-methylated | PCa\|NT DMRs |
| chr2 | 793307 | 795479 | 0.000840726 | −23.87037037 | Hypo-methylated | PCa\|NT DMRs |
| chr2 | 875812 | 876008 | 9.97E−07 | −21.22285714 | Hypo-methylated | PCa\|NT DMRs |
| chr2 | 1591015 | 1593315 | 8.23E−05 | 25.76266667 | Hyper-methylated | PCa\|NT DMRs |
| chr2 | 1609014 | 1613663 | 0.000258503 | 29.47985348 | Hyper-methylated | PCa\|NT DMRs |
| chr2 | 1628029 | 1629157 | 0.002750151 | −33.37174603 | Hypo-methylated | PCa\|NT DMRs |
| chr2 | 1679317 | 1679866 | 6.42E−05 | −56.53968254 | Hypo-methylated | PCa\|NT DMRs |
| chr2 | 1771043 | 1772199 | 0.001152293 | 24.00571429 | Hyper-methylated | PCa\|NT DMRs |
| chr2 | 1797185 | 1797682 | 0.00057866 | −21.25047619 | Hypo-methylated | PCa\|NT DMRs |
| chr2 | 1839428 | 1839689 | 0.005968871 | −54.06190476 | Hypo-methylated | PCa\|NT DMRs |
| chr2 | 1869405 | 1873216 | 0.000754912 | −24.50571429 | Hypo-methylated | PCa\|NT DMRs |
| chr2 | 1880304 | 1893255 | 6.21E−07 | −22.84901961 | Hypo-methylated | PCa\|NT DMRs |
| chr2 | 1896195 | 1914113 | 5.13E−15 | −25.7469246 | Hypo-methylated | PCa\|NT DMRs |
| chr2 | 1999403 | 1999862 | 0.000205174 | 28.85714286 | Hyper-methylated | PCa\|NT DMRs |
| chr2 | 2007603 | 2018579 | 0.001969641 | −27.55714286 | Hypo-methylated | PCa\|NT DMRs |
| chr2 | 2189948 | 2190029 | 1.42E−05 | −32.26360544 | Hypo-methylated | PCa\|NT DMRs |
| chr2 | 2295453 | 2404659 | 9.60E−07 | −25.5168254 | Hypo-methylated | PCa\|NT DMRs |
| chr2 | 2414861 | 2542083 | 3.32E−15 | −29.53874092 | Hypo-methylated | PCa\|NT DMRs |
| chr2 | 2581317 | 2604372 | 4.85E−13 | −31.88950216 | Hypo-methylated | PCa\|NT DMRs |
| chr2 | 2617176 | 2645948 | 5.78E−06 | −22.20675526 | Hypo-methylated | PCa\|NT DMRs |
| chr2 | 2666095 | 2667656 | 0.000384643 | −32.58125 | Hypo-methylated | PCa\|NT DMRs |
| chr2 | 2673279 | 2697612 | 2.88E−09 | −25.39968254 | Hypo-methylated | PCa\|NT DMRs |
| chr2 | 2712829 | 2725315 | 9.78E−12 | −32.51303258 | Hypo-methylated | PCa\|NT DMRs |
| chr2 | 2881302 | 2893716 | 2.86E−05 | −32.07216117 | Hypo-methylated | PCa\|NT DMRs |
| chr2 | 3352486 | 3367076 | 1.57E−07 | −40.09269841 | Hypo-methylated | PCa\|NT DMRs |
| chr2 | 3628198 | 3633408 | 1.03E−05 | −47.90595238 | Hypo-methylated | PCa\|NT DMRs |
| chr2 | 3827278 | 3827480 | 0.00064571 | −31.93877551 | Hypo-methylated | PCa\|NT DMRs |
| chr2 | 4976712 | 4976871 | 0.004095673 | −51.85714286 | Hypo-methylated | PCa\|NT DMRs |
| chr2 | 5582807 | 5583012 | 0.007959104 | −45.16326531 | Hypo-methylated | PCa\|NT DMRs |
| chr2 | 6072686 | 6072920 | 0.00064571 | −58.97959184 | Hypo-methylated | PCa\|NT DMRs |
| chr2 | 6121557 | 6121728 | 0.007959104 | 26.30544218 | Hyper-methylated | PCa\|NT DMRs |
| chr2 | 7571040 | 7571792 | 5.50E−18 | 27.36613011 | Hyper-methylated | PCa\|NT DMRs |
| chr2 | 8275330 | 8275400 | 0.001969641 | 20.47619048 | Hyper-methylated | PCa\|NT DMRs |
| chr2 | 8595402 | 8597487 | 4.59E−07 | 40.8989899 | Hyper-methylated | PCa\|NT DMRs |
| chr2 | 8613061 | 8617825 | 0.001969641 | 51.67857143 | Hyper-methylated | PCa\|NT DMRs |
| chr2 | 8767329 | 8767651 | 0.000651745 | −39.86904762 | Hypo-methylated | PCa\|NT DMRs |
| chr2 | 8804887 | 8805223 | 0.000228994 | 31.70684524 | Hyper-methylated | PCa\|NT DMRs |
| chr2 | 9229989 | 9236237 | 4.36E−06 | 34.61830357 | Hyper-methylated | PCa\|NT DMRs |
| chr2 | 9937836 | 9938039 | 0.001969641 | −45.47936508 | Hypo-methylated | PCa\|NT DMRs |
| chr2 | 9956025 | 9964206 | 0.003448944 | 23.70884354 | Hyper-methylated | PCa\|NT DMRs |
| chr2 | 10168865 | 10169784 | 0.005938065 | 24.05714286 | Hyper-methylated | PCa\|NT DMRs |
| chr2 | 10182542 | 10183283 | 1.03E−06 | 25.11885714 | Hyper-methylated | PCa\|NT DMRs |
| chr2 | 10196817 | 10196899 | 0.001969641 | −52.61111111 | Hypo-methylated | PCa\|NT DMRs |
| chr2 | 10200402 | 10200648 | 0.007628253 | −34.83673469 | Hypo-methylated | PCa\|NT DMRs |
| chr2 | 10220817 | 10220994 | 5.01E−06 | 21.55166667 | Hyper-methylated | PCa\|NT DMRs |
| chr2 | 10367948 | 10369419 | 0.001291665 | −41.91168831 | Hypo-methylated | PCa\|NT DMRs |
| chr2 | 10529276 | 10536699 | 0.001899053 | 20.09467787 | Hyper-methylated | PCa\|NT DMRs |
| chr2 | 10541049 | 10546988 | 2.33E−07 | 24.88275418 | Hyper-methylated | PCa\|NT DMRs |
| chr2 | 10550009 | 10550372 | 0.000205174 | 44.45654762 | Hyper-methylated | PCa\|NT DMRs |
| chr2 | 10570707 | 10573271 | 1.34E−07 | 33.54518272 | Hyper-methylated | PCa\|NT DMRs |
| chr2 | 11054446 | 11054665 | 0.005968871 | 22.32857143 | Hyper-methylated | PCa\|NT DMRs |
| chr2 | 11058934 | 11059334 | 0.003272154 | −25.09821429 | Hypo-methylated | PCa\|NT DMRs |
| chr2 | 11087220 | 11087315 | 0.001969641 | 34.0968254 | Hyper-methylated | PCa\|NT DMRs |
| chr2 | 11117281 | 11117480 | 0.000117699 | 39.35064935 | Hyper-methylated | PCa\|NT DMRs |
| chr2 | 11269676 | 11269781 | 0.00064571 | 44.9537415 | Hyper-methylated | PCa\|NT DMRs |
| chr2 | 11273541 | 11294839 | 0.000694428 | 20.65901361 | Hyper-methylated | PCa\|NT DMRs |
| chr2 | 11539045 | 11539312 | 0.003448944 | −31.57959184 | Hypo-methylated | PCa\|NT DMRs |
| chr2 | 11794703 | 11797035 | 1.76E−06 | −47.14285714 | Hypo-methylated | PCa\|NT DMRs |
| chr2 | 11816545 | 11816928 | 0.00064571 | −31.00884354 | Hypo-methylated | PCa\|NT DMRs |
| chr2 | 11817063 | 11818013 | 0.000144451 | 26.43465608 | Hyper-methylated | PCa\|NT DMRs |
| chr2 | 11822852 | 11823131 | 0.002767976 | −28.02597403 | Hypo-methylated | PCa\|NT DMRs |
| chr2 | 11990386 | 11990468 | 0.000377317 | −22.75892857 | Hypo-methylated | PCa\|NT DMRs |
| chr2 | 12083451 | 12124397 | 0.005968871 | −20.42857143 | Hypo-methylated | PCa\|NT DMRs |
| chr2 | 12858923 | 12859080 | 1.68E−06 | 32.37310924 | Hyper-methylated | PCa\|NT DMRs |
| chr2 | 15174632 | 15181949 | 0.005513661 | −26.76488095 | Hypo-methylated | PCa\|NT DMRs |
| chr2 | 15909180 | 15910005 | 0.001115302 | 26.37797619 | Hyper-methylated | PCa\|NT DMRs |
| chr2 | 15925398 | 15928759 | 5.06E−06 | −26.0070028 | Hypo-methylated | PCa\|NT DMRs |
| chr2 | 16082898 | 16089407 | 0.002668449 | 20.87843137 | Hyper-methylated | PCa\|NT DMRs |
| chr2 | 16386194 | 16448084 | 0.000355892 | −26.4 | Hypo-methylated | PCa\|NT DMRs |
| chr2 | 16744057 | 16780770 | 0.000205174 | −21.00595238 | Hypo-methylated | PCa\|NT DMRs |
| chr2 | 20068429 | 20068953 | 2.52E−13 | 38.86446886 | Hyper-methylated | PCa\|NT DMRs |
| chr2 | 20385350 | 20386297 | 0.000974795 | 27.31785714 | Hyper-methylated | PCa\|NT DMRs |
| chr2 | 20396474 | 20396637 | 1.97E−05 | −52.62142857 | Hypo-methylated | PCa\|NT DMRs |
| chr2 | 20412821 | 20413626 | 0.004095673 | −55.71269841 | Hypo-methylated | PCa\|NT DMRs |
| chr2 | 20644948 | 20646182 | 0.002773417 | 21.29444444 | Hyper-methylated | PCa\|NT DMRs |
| chr2 | 20647265 | 20648260 | 8.59E−07 | −27.25666667 | Hypo-methylated | PCa\|NT DMRs |
| chr2 | 21925827 | 22114299 | 1.52E−07 | −32.10612245 | Hypo-methylated | PCa\|NT DMRs |
| chr2 | 22307033 | 23000777 | 5.18E−21 | −31.68116883 | Hypo-methylated | PCa\|NT DMRs |
| chr2 | 23178425 | 23232896 | 0.002373155 | −33.46825397 | Hypo-methylated | PCa\|NT DMRs |
| chr2 | 23881541 | 23886385 | 0.000101817 | 22.41269841 | Hyper-methylated | PCa\|NT DMRs |
| chr2 | 23936461 | 24015758 | 0.009382828 | 33.77460317 | Hyper-methylated | PCa\|NT DMRs |
| chr2 | 24272701 | 24273290 | 1.92E−12 | 22.83521595 | Hyper-methylated | PCa\|NT DMRs |
| chr2 | 24397760 | 24398064 | 4.01E−08 | 26.50746032 | Hyper-methylated | PCa\|NT DMRs |
| chr2 | 25023163 | 25038618 | 0.001394173 | −34.61530612 | Hypo-methylated | PCa\|NT DMRs |
| chr2 | 25097021 | 25109925 | 4.63E−05 | 24.87563025 | Hyper-methylated | PCa\|NT DMRs |
| chr2 | 25149334 | 25151402 | 2.81E−06 | 41.5007619 | Hyper-methylated | PCa\|NT DMRs |
| chr2 | 25209069 | 25220341 | 0.001969641 | −29.17857143 | Hypo-methylated | PCa\|NT DMRs |
| chr2 | 25425302 | 25427514 | 6.86E−18 | 39.06543779 | Hyper-methylated | PCa\|NT DMRs |
| chr2 | 25799754 | 25800026 | 0.00179283 | −39.09821429 | Hypo-methylated | PCa\|NT DMRs |
| chr2 | 25805082 | 25805160 | 0.005968871 | 32.81428571 | Hyper-methylated | PCa\|NT DMRs |
| chr2 | 26045138 | 26045177 | 0.005968871 | 25.80952381 | Hyper-methylated | PCa\|NT DMRs |
| chr2 | 26139498 | 26139524 | 0.008482405 | 57.54285714 | Hyper-methylated | PCa\|NT DMRs |
| chr2 | 26204700 | 26204995 | 5.06E−06 | 29.79551821 | Hyper-methylated | PCa\|NT DMRs |
| chr2 | 26394194 | 26395683 | 0.000220386 | 25.21137026 | Hyper-methylated | PCa\|NT DMRs |
| chr2 | 26521360 | 26523216 | 2.01E−07 | 45.51071429 | Hyper-methylated | PCa\|NT DMRs |
| chr2 | 26624616 | 26625140 | 7.76E−14 | 34.73571429 | Hyper-methylated | PCa\|NT DMRs |
| chr2 | 26700315 | 26700393 | 0.004037929 | 38.83673469 | Hyper-methylated | PCa\|NT DMRs |
| chr2 | 26872310 | 26872729 | 0.000339627 | −44.02777778 | Hypo-methylated | PCa\|NT DMRs |
| chr2 | 27045085 | 27068314 | 0.00023371 | −20.89914966 | Hypo-methylated | PCa\|NT DMRs |
| chr2 | 27072408 | 27072835 | 3.91E−13 | 21.25736961 | Hyper-methylated | PCa\|NT DMRs |
| chr2 | 27194059 | 27194443 | 0.00064571 | 24.96258503 | Hyper-methylated | PCa\|NT DMRs |
| chr2 | 27529119 | 27530773 | 2.26E−27 | 36.86595945 | Hyper-methylated | PCa\|NT DMRs |
| chr2 | 27599189 | 27601856 | 0.007177823 | −24.13613445 | Hypo-methylated | PCa\|NT DMRs |
| chr2 | 27665129 | 27665651 | 2.05E−17 | −33.5462963 | Hypo-methylated | PCa\|NT DMRs |
| chr2 | 27953589 | 27960870 | 1.03E−08 | 36.57380952 | Hyper-methylated | PCa\|NT DMRs |
| chr2 | 28633060 | 28633406 | 0.000354065 | 51.36507937 | Hyper-methylated | PCa\|NT DMRs |
| chr2 | 28976750 | 28976898 | 0.004947537 | −25.11111111 | Hypo-methylated | PCa\|NT DMRs |
| chr2 | 29033353 | 29034134 | 3.51E−13 | 26.9191342 | Hyper-methylated | PCa\|NT DMRs |
| chr2 | 29338031 | 29338356 | 8.16E−17 | 23.01923077 | Hyper-methylated | PCa\|NT DMRs |
| chr2 | 30455541 | 30455705 | 1.64E−06 | 20.06890756 | Hyper-methylated | PCa\|NT DMRs |
| chr2 | 30559925 | 30561092 | 0.002767976 | 23.35670996 | Hyper-methylated | PCa\|NT DMRs |
| chr2 | 30885534 | 30885833 | 0.000205174 | −52.87559524 | Hypo-methylated | PCa\|NT DMRs |
| chr2 | 30998710 | 30999051 | 0.001344447 | 49.89880952 | Hyper-methylated | PCa\|NT DMRs |
| chr2 | 31942306 | 31962425 | 0.005968871 | −20.1752381 | Hypo-methylated | PCa\|NT DMRs |
| chr2 | 33532846 | 33533035 | 0.005968871 | −53.06666667 | Hypo-methylated | PCa\|NT DMRs |
| chr2 | 36041722 | 36528260 | 7.69E−11 | −25.96152381 | Hypo-methylated | PCa\|NT DMRs |
| chr2 | 38300862 | 38301774 | 5.29E−23 | 30.02061224 | Hyper-methylated | PCa\|NT DMRs |
| chr2 | 38827180 | 38827869 | 0.005968871 | −26.81216931 | Hypo-methylated | PCa\|NT DMRs |
| chr2 | 39186938 | 39187433 | 1.15E−05 | 21.67746032 | Hyper-methylated | PCa\|NT DMRs |
| chr2 | 39472228 | 39472654 | 5.84E−05 | 44.60714286 | Hyper-methylated | PCa\|NT DMRs |
| chr2 | 39892612 | 39892964 | 9.62E−06 | 26.81534392 | Hyper-methylated | PCa\|NT DMRs |
| chr2 | 40225200 | 40270225 | 0.000377317 | −25.82083333 | Hypo-methylated | PCa\|NT DMRs |
| chr2 | 40498961 | 40558601 | 4.84E−06 | −22.95029762 | Hypo-methylated | PCa\|NT DMRs |
| chr2 | 42106019 | 42106107 | 0.008482405 | 46.54285714 | Hyper-methylated | PCa\|NT DMRs |
| chr2 | 43132698 | 43133093 | 0.000366497 | 30.02380952 | Hyper-methylated | PCa\|NT DMRs |
| chr2 | 43153331 | 43153629 | 0.001969641 | −57.66031746 | Hypo-methylated | PCa\|NT DMRs |
| chr2 | 43161979 | 43165586 | 0.002085197 | 22.03174603 | Hyper-methylated | PCa\|NT DMRs |
| chr2 | 43228043 | 43233625 | 0.008581782 | 24.20982143 | Hyper-methylated | PCa\|NT DMRs |
| chr2 | 43286160 | 43294887 | 0.000481535 | 28.88095238 | Hyper-methylated | PCa\|NT DMRs |
| chr2 | 43396962 | 43405569 | 2.40E−07 | 46.00421245 | Hyper-methylated | PCa\|NT DMRs |
| chr2 | 44933808 | 45014780 | 1.77E−05 | 29.78761905 | Hyper-methylated | PCa\|NT DMRs |
| chr2 | 45149217 | 45155504 | 8.34E−07 | 25.54679803 | Hyper-methylated | PCa\|NT DMRs |
| chr2 | 45163020 | 45165772 | 3.88E−10 | 28.48441558 | Hyper-methylated | PCa\|NT DMRs |
| chr2 | 45396723 | 45398172 | 3.52E−12 | 31.25136054 | Hyper-methylated | PCa\|NT DMRs |
| chr2 | 45485264 | 45485490 | 0.00046742 | 36.19480519 | Hyper-methylated | PCa\|NT DMRs |
| chr2 | 45501627 | 45501894 | 0.001969641 | −48.57857143 | Hypo-methylated | PCa\|NT DMRs |
| chr2 | 46523506 | 46523905 | 0.003450017 | 20.29828571 | Hyper-methylated | PCa\|NT DMRs |
| chr2 | 46558860 | 46562940 | 0.001391421 | −24.51082251 | Hypo-methylated | PCa\|NT DMRs |
| chr2 | 46770499 | 46770976 | 2.42E−07 | 29.98302277 | Hyper-methylated | PCa\|NT DMRs |
| chr2 | 47206494 | 47210755 | 1.11E−05 | 54.75324675 | Hyper-methylated | PCa\|NT DMRs |
| chr2 | 48209009 | 48316147 | 4.77E−05 | −22.17482993 | Hypo-methylated | PCa\|NT DMRs |
| chr2 | 48994644 | 49054084 | 1.77E−05 | −32.62142857 | Hypo-methylated | PCa\|NT DMRs |
| chr2 | 49143214 | 49160870 | 1.42E−05 | −31.41326531 | Hypo-methylated | PCa\|NT DMRs |
| chr2 | 49222362 | 49345604 | 0.001969641 | −36.1031746 | Hypo-methylated | PCa\|NT DMRs |
| chr2 | 49751772 | 49800742 | 0.000392715 | −26.12380952 | Hypo-methylated | PCa\|NT DMRs |
| chr2 | 52799450 | 53537533 | 1.96E−30 | −28.5737646 | Hypo-methylated | PCa\|NT DMRs |
| chr2 | 54900534 | 54901150 | 4.90E−06 | 42.02678571 | Hyper-methylated | PCa\|NT DMRs |
| chr2 | 55349410 | 55381244 | 0.002713936 | 33.16883117 | Hyper-methylated | PCa\|NT DMRs |
| chr2 | 55960177 | 56022539 | 1.52E−07 | −24.69897959 | Hypo-methylated | PCa\|NT DMRs |
| chr2 | 56238691 | 56410072 | 0.00064571 | −33.15102041 | Hypo-methylated | PCa\|NT DMRs |
| chr2 | 56420144 | 58077786 | 2.52E−30 | −32.34753247 | Hypo-methylated | PCa\|NT DMRs |
| chr2 | 58478744 | 58654887 | 2.42E−06 | −30.5802381 | Hypo-methylated | PCa\|NT DMRs |
| chr2 | 58751739 | 59288266 | 3.34E−15 | −37.03481481 | Hypo-methylated | PCa\|NT DMRs |
| chr2 | 59294363 | 59580792 | 3.90E−09 | −36.65996473 | Hypo-methylated | PCa\|NT DMRs |
| chr2 | 60666901 | 60667013 | 0.001115302 | 32.71845238 | Hyper-methylated | PCa\|NT DMRs |
| chr2 | 60674391 | 60687915 | 4.45E−08 | −29.43079365 | Hypo-methylated | PCa\|NT DMRs |
| chr2 | 62520450 | 62521440 | 0.001969641 | −32.73190476 | Hypo-methylated | PCa\|NT DMRs |
| chr2 | 64291259 | 64291471 | 0.008482405 | −55.42857143 | Hypo-methylated | PCa\|NT DMRs |
| chr2 | 64682113 | 64682294 | 0.003544378 | −31.91428571 | Hypo-methylated | PCa\|NT DMRs |
| chr2 | 64834126 | 64834341 | 0.001969641 | 42.64285714 | Hyper-methylated | PCa\|NT DMRs |
| chr2 | 64836278 | 64836819 | 1.10E−14 | 23.87319902 | Hyper-methylated | PCa\|NT DMRs |
| chr2 | 64863610 | 64863692 | 6.42E−05 | 51.58730159 | Hyper-methylated | PCa\|NT DMRs |
| chr2 | 65173463 | 65173694 | 0.005968871 | 58.09047619 | Hyper-methylated | PCa\|NT DMRs |
| chr2 | 65793940 | 65794008 | 0.001969641 | 38.5 | Hyper-methylated | PCa\|NT DMRs |
| chr2 | 65958699 | 65958944 | 0.005968871 | −24.0952381 | Hypo-methylated | PCa\|NT DMRs |
| chr2 | 66096573 | 66309102 | 4.59E−06 | −20.42335601 | Hypo-methylated | PCa\|NT DMRs |
| chr2 | 67788644 | 67971406 | 0.003448944 | −47.26190476 | Hypo-methylated | PCa\|NT DMRs |
| chr2 | 69001774 | 69002219 | 6.63E−10 | 43.81734694 | Hyper-methylated | PCa\|NT DMRs |
| chr2 | 69524089 | 69533971 | 3.27E−06 | 20.71333333 | Hyper-methylated | PCa\|NT DMRs |
| chr2 | 70202705 | 70205900 | 0.000396159 | 34.85 | Hyper-methylated | PCa\|NT DMRs |
| chr2 | 70905810 | 70905975 | 0.001969641 | −28.97959184 | Hypo-methylated | PCa\|NT DMRs |
| chr2 | 71192319 | 71192453 | 0.000116533 | 25.08225108 | Hyper-methylated | PCa\|NT DMRs |
| chr2 | 71271947 | 71272816 | 7.19E−05 | −33.53571429 | Hypo-methylated | PCa\|NT DMRs |
| chr2 | 71692287 | 71693328 | 0.001037738 | 28.00285714 | Hyper-methylated | PCa\|NT DMRs |
| chr2 | 71832191 | 71832252 | 0.005968871 | 41.6 | Hyper-methylated | PCa\|NT DMRs |
| chr2 | 71927359 | 71928137 | 0.006738891 | −25.66598639 | Hypo-methylated | PCa\|NT DMRs |
| chr2 | 71944105 | 71944338 | 0.000205174 | −43.95833333 | Hypo-methylated | PCa\|NT DMRs |
| chr2 | 71974171 | 71974392 | 0.000481535 | −24.68253968 | Hypo-methylated | PCa\|NT DMRs |
| chr2 | 72012720 | 72013043 | 0.000215808 | 32.92328042 | Hyper-methylated | PCa\|NT DMRs |
| chr2 | 72369534 | 72371794 | 1.83E−11 | 32.8714966 | Hyper-methylated | PCa\|NT DMRs |
| chr2 | 73512124 | 73512214 | 0.003892841 | 29.73015873 | Hyper-methylated | PCa\|NT DMRs |
| chr2 | 73551380 | 73605217 | 0.003105416 | −26.403663 | Hypo-methylated | PCa\|NT DMRs |
| chr2 | 73931532 | 73931829 | 0.004005318 | 49.97619048 | Hyper-methylated | PCa\|NT DMRs |
| chr2 | 74138244 | 74153214 | 0.00022692 | −22.96825397 | Hypo-methylated | PCa\|NT DMRs |
| chr2 | 74252650 | 74252691 | 0.008482405 | 44.09142857 | Hyper-methylated | PCa\|NT DMRs |
| chr2 | 74412615 | 74412808 | 0.000205174 | 43.21428571 | Hyper-methylated | PCa\|NT DMRs |
| chr2 | 74641954 | 74643332 | 4.02E−10 | 31.96451914 | Hyper-methylated | PCa\|NT DMRs |
| chr2 | 74667768 | 74668636 | 0.002152552 | −21.59353741 | Hypo-methylated | PCa\|NT DMRs |
| chr2 | 74779511 | 74779629 | 0.001969641 | 44.53469388 | Hyper-methylated | PCa\|NT DMRs |
| chr2 | 75292212 | 75292273 | 0.001969641 | 45.28968254 | Hyper-methylated | PCa\|NT DMRs |
| chr2 | 76077436 | 76366398 | 7.54E−12 | −30.14414414 | Hypo-methylated | PCa\|NT DMRs |
| chr2 | 76975571 | 77061259 | 1.69E−06 | −52.71428571 | Hypo-methylated | PCa\|NT DMRs |
| chr2 | 77925980 | 77961243 | 3.51E−05 | −27.65054945 | Hypo-methylated | PCa\|NT DMRs |
| chr2 | 79006310 | 79077222 | 8.75E−09 | −27.07777778 | Hypo-methylated | PCa\|NT DMRs |
| chr2 | 79102826 | 79187496 | 6.42E−05 | −25.01798942 | Hypo-methylated | PCa\|NT DMRs |
| chr2 | 79492296 | 79684185 | 6.55E−13 | −33.63675214 | Hypo-methylated | PCa\|NT DMRs |
| chr2 | 79779504 | 79897577 | 7.12E−08 | −31.03152709 | Hypo-methylated | PCa\|NT DMRs |
| chr2 | 80334595 | 80494816 | 7.80E−07 | −34.53257143 | Hypo-methylated | PCa\|NT DMRs |
| chr2 | 80557949 | 80773063 | 2.15E−08 | −38.6127619 | Hypo-methylated | PCa\|NT DMRs |
| chr2 | 81098971 | 81128010 | 0.000205174 | −40.40178571 | Hypo-methylated | PCa\|NT DMRs |
| chr2 | 84102833 | 84522159 | 2.54E−16 | −33.18380952 | Hypo-methylated | PCa\|NT DMRs |
| chr2 | 85092756 | 85098904 | 0.00641359 | −37.42857143 | Hypo-methylated | PCa\|NT DMRs |
| chr2 | 85106954 | 85107452 | 6.94E−06 | 40.72952381 | Hyper-methylated | PCa\|NT DMRs |
| chr2 | 85361978 | 85362295 | 3.44E−06 | 38.5625 | Hyper-methylated | PCa\|NT DMRs |
| chr2 | 85640740 | 85644803 | 1.24E−06 | 27.3577573 | Hyper-methylated | PCa\|NT DMRs |
| chr2 | 85888390 | 85888724 | 0.001969641 | 22.13809524 | Hyper-methylated | PCa\|NT DMRs |
| chr2 | 85938636 | 85949493 | 0.000200126 | 35.42155388 | Hyper-methylated | PCa\|NT DMRs |
| chr2 | 85975464 | 85980817 | 6.13E−05 | 30.80912698 | Hyper-methylated | PCa\|NT DMRs |
| chr2 | 86001690 | 86017953 | 0.003410476 | 24.61724138 | Hyper-methylated | PCa\|NT DMRs |
| chr2 | 86245145 | 86248352 | 0.000908257 | −35.01318681 | Hypo-methylated | PCa\|NT DMRs |
| chr2 | 87581832 | 87743769 | 1.28E−07 | −37.84246032 | Hypo-methylated | PCa\|NT DMRs |
| chr2 | 88248026 | 88301765 | 0.001471804 | −26.25793651 | Hypo-methylated | PCa\|NT DMRs |
| chr2 | 88591069 | 88649189 | 1.73E−08 | −40.49047619 | Hypo-methylated | PCa\|NT DMRs |
| chr2 | 88756044 | 88791818 | 2.79E−08 | −33.61904762 | Hypo-methylated | PCa\|NT DMRs |
| chr2 | 90469037 | 90485802 | 1.22E−06 | −28.98571429 | Hypo-methylated | PCa\|NT DMRs |
| chr2 | 91678864 | 91698073 | 0.000435385 | −33.4003663 | Hypo-methylated | PCa\|NT DMRs |
| chr2 | 91706426 | 91755271 | 0.005968871 | −33.14285714 | Hypo-methylated | PCa\|NT DMRs |
| chr2 | 91940484 | 91957149 | 0.003032307 | −29.10578231 | Hypo-methylated | PCa\|NT DMRs |
| chr2 | 95523816 | 95524163 | 8.42E−05 | 28.52380952 | Hyper-methylated | PCa\|NT DMRs |
| chr2 | 95539703 | 95539830 | 0.00179283 | −27.50714286 | Hypo-methylated | PCa\|NT DMRs |
| chr2 | 95692274 | 95693115 | 7.43E−07 | 23.28154762 | Hyper-methylated | PCa\|NT DMRs |
| chr2 | 95884907 | 95899095 | 0.000205174 | −26.12142857 | Hypo-methylated | PCa\|NT DMRs |
| chr2 | 96012660 | 96013247 | 0.003095938 | 20.60345238 | Hyper-methylated | PCa\|NT DMRs |
| chr2 | 96829878 | 96830574 | 0.001042833 | 23.28571429 | Hyper-methylated | PCa\|NT DMRs |
| chr2 | 96990968 | 96991937 | 1.95E−11 | 23.34639456 | Hyper-methylated | PCa\|NT DMRs |
| chr2 | 97151767 | 97152249 | 0.000205174 | 39.15357143 | Hyper-methylated | PCa\|NT DMRs |
| chr2 | 97174350 | 97191875 | 0.000600104 | −26.98129252 | Hypo-methylated | PCa\|NT DMRs |
| chr2 | 97203371 | 97203683 | 2.58E−05 | 26.6970696 | Hyper-methylated | PCa\|NT DMRs |
| chr2 | 97504837 | 97505294 | 0.001060348 | 42.37662338 | Hyper-methylated | PCa\|NT DMRs |
| chr2 | 97532102 | 97532830 | 0.000102305 | 32.36825397 | Hyper-methylated | PCa\|NT DMRs |
| chr2 | 97533635 | 97534300 | 1.47E−05 | 21.70907029 | Hyper-methylated | PCa\|NT DMRs |
| chr2 | 98329337 | 98330350 | 1.24E−07 | 29.11309524 | Hyper-methylated | PCa\|NT DMRs |
| chr2 | 98896930 | 98897302 | 0.00179283 | 27.63988095 | Hyper-methylated | PCa\|NT DMRs |
| chr2 | 99796276 | 99796456 | 0.001969641 | −43.72619048 | Hypo-methylated | PCa\|NT DMRs |
| chr2 | 100186071 | 100189473 | 0.00064571 | −24.11020408 | Hypo-methylated | PCa\|NT DMRs |
| chr2 | 100722012 | 100763757 | 0.008083098 | −23.62063492 | Hypo-methylated | PCa\|NT DMRs |
| chr2 | 100851655 | 100851922 | 0.000205174 | 53.55297619 | Hyper-methylated | PCa\|NT DMRs |
| chr2 | 100956434 | 100998861 | 0.001969641 | −25.95952381 | Hypo-methylated | PCa\|NT DMRs |
| chr2 | 101619642 | 101619885 | 0.007959104 | −37.81632653 | Hypo-methylated | PCa\|NT DMRs |
| chr2 | 101926330 | 101929961 | 0.001969641 | 37.37301587 | Hyper-methylated | PCa\|NT DMRs |
| chr2 | 103464975 | 103465123 | 5.78E−05 | 29.03571429 | Hyper-methylated | PCa\|NT DMRs |
| chr2 | 104973286 | 105235377 | 5.04E−17 | −38.82587065 | Hypo-methylated | PCa\|NT DMRs |
| chr2 | 105853623 | 105853781 | 6.42E−05 | 50.63492063 | Hyper-methylated | PCa\|NT DMRs |
| chr2 | 105979730 | 105979901 | 0.005968871 | 46.4 | Hyper-methylated | PCa\|NT DMRs |
| chr2 | 106014832 | 106015180 | 3.04E−08 | 26.74202899 | Hyper-methylated | PCa\|NT DMRs |
| chr2 | 106548681 | 106571947 | 0.00068166 | −21.37142857 | Hypo-methylated | PCa\|NT DMRs |
| chr2 | 107484769 | 107485028 | 0.003622801 | −31.81428571 | Hypo-methylated | PCa\|NT DMRs |
| chr2 | 108888469 | 108889211 | 3.32E−06 | −57.38492063 | Hypo-methylated | PCa\|NT DMRs |
| chr2 | 108990108 | 108990417 | 0.00023371 | −48.78571429 | Hypo-methylated | PCa\|NT DMRs |
| chr2 | 109196276 | 109196645 | 0.000205174 | 22.85714286 | Hyper-methylated | PCa\|NT DMRs |
| chr2 | 109840995 | 109855262 | 0.001429556 | 33.6038961 | Hyper-methylated | PCa\|NT DMRs |
| chr2 | 109907927 | 109908003 | 0.000215808 | −24.03174603 | Hypo-methylated | PCa\|NT DMRs |
| chr2 | 110372895 | 110373059 | 0.000377317 | 25.03571429 | Hyper-methylated | PCa\|NT DMRs |
| chr2 | 112123978 | 112124095 | 2.73E−06 | 20.11071429 | Hyper-methylated | PCa\|NT DMRs |
| chr2 | 112897975 | 112898369 | 2.31E−07 | 33.76309524 | Hyper-methylated | PCa\|NT DMRs |
| chr2 | 112913017 | 112915980 | 0.001041221 | 30.76526611 | Hyper-methylated | PCa\|NT DMRs |
| chr2 | 113930805 | 113933877 | 0.005295761 | 30.47012987 | Hyper-methylated | PCa\|NT DMRs |
| chr2 | 113955773 | 113955973 | 0.000205174 | −29.79166667 | Hypo-methylated | PCa\|NT DMRs |
| chr2 | 114048902 | 114063733 | 0.002580892 | 38.78571429 | Hyper-methylated | PCa\|NT DMRs |
| chr2 | 115294076 | 115407087 | 7.51E−07 | −41.8015873 | Hypo-methylated | PCa\|NT DMRs |
| chr2 | 115542670 | 115918201 | 1.87E−16 | −31.58872692 | Hypo-methylated | PCa\|NT DMRs |
| chr2 | 115935859 | 117309836 | 3.44E−31 | −32.21763393 | Hypo-methylated | PCa\|NT DMRs |
| chr2 | 117602049 | 117784436 | 6.42E−05 | −35.13915344 | Hypo-methylated | PCa\|NT DMRs |
| chr2 | 119599195 | 119599642 | 0.000706751 | 22.21577381 | Hyper-methylated | PCa\|NT DMRs |
| chr2 | 119703150 | 119751859 | 2.30E−06 | −32.40136054 | Hypo-methylated | PCa\|NT DMRs |
| chr2 | 119809598 | 119902570 | 7.85E−08 | −24.8805291 | Hypo-methylated | PCa\|NT DMRs |
| chr2 | 119982015 | 119985068 | 0.009050681 | 21.67238095 | Hyper-methylated | PCa\|NT DMRs |
| chr2 | 120418595 | 120436086 | 0.003622801 | −26.52142857 | Hypo-methylated | PCa\|NT DMRs |
| chr2 | 121156865 | 121157082 | 0.000118733 | −34.4978836 | Hypo-methylated | PCa\|NT DMRs |
| chr2 | 121158454 | 121162391 | 0.003892841 | 22.23809524 | Hyper-methylated | PCa\|NT DMRs |
| chr2 | 121411913 | 121412564 | 1.48E−05 | 24.70457875 | Hyper-methylated | PCa\|NT DMRs |
| chr2 | 121499560 | 121499783 | 0.002585277 | 22.37738095 | Hyper-methylated | PCa\|NT DMRs |
| chr2 | 121624808 | 121625839 | 1.70E−07 | 20.43205575 | Hyper-methylated | PCa\|NT DMRs |
| chr2 | 121691704 | 121698626 | 0.001430886 | −29.98214286 | Hypo-methylated | PCa\|NT DMRs |
| chr2 | 121748671 | 121750909 | 0.006589535 | 22.46952381 | Hyper-methylated | PCa\|NT DMRs |
| chr2 | 121763736 | 121763822 | 0.000205174 | −58.57738095 | Hypo-methylated | PCa\|NT DMRs |
| chr2 | 121776280 | 121778871 | 0.005438982 | 21.17380952 | Hyper-methylated | PCa\|NT DMRs |
| chr2 | 121820414 | 121820577 | 0.00064571 | −40.42857143 | Hypo-methylated | PCa\|NT DMRs |
| chr2 | 122042236 | 122042484 | 2.69E−09 | 35.80842491 | Hyper-methylated | PCa\|NT DMRs |
| chr2 | 122522793 | 122522900 | 0.00641359 | 25.58888889 | Hyper-methylated | PCa\|NT DMRs |
| chr2 | 122532880 | 122545681 | 0.000205174 | −24.7797619 | Hypo-methylated | PCa\|NT DMRs |
| chr2 | 122660705 | 122720549 | 0.003847481 | −29.21804511 | Hypo-methylated | PCa\|NT DMRs |
| chr2 | 122809721 | 122809788 | 1.97E−05 | −43.13809524 | Hypo-methylated | PCa\|NT DMRs |
| chr2 | 123989564 | 124002963 | 0.001969641 | −26.78095238 | Hypo-methylated | PCa\|NT DMRs |
| chr2 | 124669211 | 124782299 | 8.93E−06 | 28.88971429 | Hyper-methylated | PCa\|NT DMRs |
| chr2 | 127536795 | 127643495 | 6.87E−21 | −34.39964986 | Hypo-methylated | PCa\|NT DMRs |
| chr2 | 127643858 | 127643939 | 0.001181579 | 21.2755102 | Hyper-methylated | PCa\|NT DMRs |
| chr2 | 127644612 | 127695655 | 6.21E−05 | −38.25612245 | Hypo-methylated | PCa\|NT DMRs |
| chr2 | 127734072 | 127754753 | 5.33E−05 | −29.4021978 | Hypo-methylated | PCa\|NT DMRs |
| chr2 | 127770008 | 127770465 | 0.005968871 | −38.1952381 | Hypo-methylated | PCa\|NT DMRs |
| chr2 | 127836137 | 127839768 | 0.002843063 | 32.9985119 | Hyper-methylated | PCa\|NT DMRs |
| chr2 | 127844776 | 127845178 | 0.00064571 | 35.13605442 | Hyper-methylated | PCa\|NT DMRs |
| chr2 | 127933204 | 127934921 | 0.000532147 | −20.97857143 | Hypo-methylated | PCa\|NT DMRs |
| chr2 | 127950803 | 127955560 | 0.00998923 | −21.05625 | Hypo-methylated | PCa\|NT DMRs |
| chr2 | 128340902 | 128341171 | 0.007959104 | −22.63741497 | Hypo-methylated | PCa\|NT DMRs |
| chr2 | 128432170 | 128432409 | 0.001924648 | 27.57922078 | Hyper-methylated | PCa\|NT DMRs |
| chr2 | 129037458 | 129037906 | 0.000481535 | 56.47566138 | Hyper-methylated | PCa\|NT DMRs |
| chr2 | 129492225 | 129492818 | 0.000205174 | 49.71428571 | Hyper-methylated | PCa\|NT DMRs |
| chr2 | 130952660 | 130952745 | 0.005968871 | −51.96190476 | Hypo-methylated | PCa\|NT DMRs |
| chr2 | 131594992 | 131599146 | 0.001860358 | 20.20984127 | Hyper-methylated | PCa\|NT DMRs |
| chr2 | 131723528 | 131724245 | 0.003448944 | 20.6122449 | Hyper-methylated | PCa\|NT DMRs |
| chr2 | 131875716 | 131884270 | 0.005968871 | −39.05714286 | Hypo-methylated | PCa\|NT DMRs |
| chr2 | 132056960 | 132109945 | 0.005513661 | 21.16296296 | Hyper-methylated | PCa\|NT DMRs |
| chr2 | 132236936 | 132237183 | 0.00064571 | −52.07619048 | Hypo-methylated | PCa\|NT DMRs |
| chr2 | 132559025 | 132559272 | 0.006410012 | −21.43040293 | Hypo-methylated | PCa\|NT DMRs |
| chr2 | 132589570 | 132589637 | 0.002097193 | 25.56802721 | Hyper-methylated | PCa\|NT DMRs |
| chr2 | 132795209 | 132795258 | 0.008482405 | 42.68571429 | Hyper-methylated | PCa\|NT DMRs |
| chr2 | 133015816 | 133025559 | 2.88E−06 | −21.17628766 | Hypo-methylated | PCa\|NT DMRs |
| chr2 | 133025978 | 133029690 | 0.000260619 | −23.1389234 | Hypo-methylated | PCa\|NT DMRs |
| chr2 | 133428721 | 133456173 | 1.31E−05 | 29.52748918 | Hyper-methylated | PCa\|NT DMRs |
| chr2 | 134657449 | 134776379 | 0.000117039 | −28.76666667 | Hypo-methylated | PCa\|NT DMRs |
| chr2 | 134928727 | 135025810 | 0.008581782 | 25.45436508 | Hyper-methylated | PCa\|NT DMRs |
| chr2 | 137164351 | 137172836 | 0.000822388 | −24.29393939 | Hypo-methylated | PCa\|NT DMRs |
| chr2 | 137244040 | 137343060 | 0.005302622 | −22.42232143 | Hypo-methylated | PCa\|NT DMRs |
| chr2 | 137354387 | 137411341 | 0.001969641 | −37.07142857 | Hypo-methylated | PCa\|NT DMRs |
| chr2 | 137543069 | 138417393 | 5.62E−15 | −30.99515599 | Hypo-methylated | PCa\|NT DMRs |
| chr2 | 140607151 | 142022624 | 3.97E−25 | −26.06710398 | Hypo-methylated | PCa\|NT DMRs |
| chr2 | 145281942 | 145281994 | 1.76E−06 | 33.45833333 | Hyper-methylated | PCa\|NT DMRs |
| chr2 | 146597676 | 147298861 | 8.85E−16 | −27.32891156 | Hypo-methylated | PCa\|NT DMRs |
| chr2 | 149854919 | 149859916 | 0.001111028 | −20.55555556 | Hypo-methylated | PCa\|NT DMRs |
| chr2 | 150176902 | 150177278 | 1.56E−08 | 26.61904762 | Hyper-methylated | PCa\|NT DMRs |
| chr2 | 151281772 | 151282012 | 0.005968871 | 29.86190476 | Hyper-methylated | PCa\|NT DMRs |
| chr2 | 153797945 | 154113653 | 4.78E−07 | −26.7689441 | Hypo-methylated | PCa\|NT DMRs |
| chr2 | 154634412 | 154675055 | 0.000840726 | −20.85873016 | Hypo-methylated | PCa\|NT DMRs |
| chr2 | 155554932 | 155556069 | 9.47E−07 | 31.3859127 | Hyper-methylated | PCa\|NT DMRs |
| chr2 | 156456945 | 156457000 | 0.000205174 | −37.96071429 | Hypo-methylated | PCa\|NT DMRs |
| chr2 | 157179350 | 157186447 | 1.85E−31 | 42.20275229 | Hyper-methylated | PCa\|NT DMRs |
| chr2 | 158607880 | 158607992 | 0.005968871 | −39.39619048 | Hypo-methylated | PCa\|NT DMRs |
| chr2 | 162095496 | 162101550 | 4.97E−08 | 20.83281573 | Hyper-methylated | PCa\|NT DMRs |
| chr2 | 162115207 | 162138207 | 0.001583613 | −23.71731602 | Hypo-methylated | PCa\|NT DMRs |
| chr2 | 162301847 | 162311737 | 4.30E−05 | −25.19413919 | Hypo-methylated | PCa\|NT DMRs |
| chr2 | 162482247 | 162482398 | 0.001969641 | 39.5952381 | Hyper-methylated | PCa\|NT DMRs |
| chr2 | 165811678 | 165851629 | 0.001089765 | −25.2359944 | Hypo-methylated | PCa\|NT DMRs |
| chr2 | 167088223 | 167225136 | 1.97E−05 | −32.36666667 | Hypo-methylated | PCa\|NT DMRs |
| chr2 | 167271040 | 167406725 | 0.003892841 | −33.3047619 | Hypo-methylated | PCa\|NT DMRs |
| chr2 | 167594864 | 167622982 | 0.002534928 | −33.74047619 | Hypo-methylated | PCa\|NT DMRs |
| chr2 | 169585372 | 169585445 | 0.005968871 | −29.2 | Hypo-methylated | PCa\|NT DMRs |
| chr2 | 170563283 | 170573652 | 0.000266788 | −38.15638095 | Hypo-methylated | PCa\|NT DMRs |
| chr2 | 172972302 | 172974226 | 1.03E−09 | 40.29130435 | Hyper-methylated | PCa\|NT DMRs |
| chr2 | 173330355 | 173330425 | 0.000117507 | −54.07792208 | Hypo-methylated | PCa\|NT DMRs |
| chr2 | 174313921 | 174314045 | 0.00576331 | −20.28869048 | Hypo-methylated | PCa\|NT DMRs |
| chr2 | 174397678 | 174448404 | 0.000339757 | −31.91125541 | Hypo-methylated | PCa\|NT DMRs |
| chr2 | 174877326 | 174877762 | 6.73E−08 | 25.17354497 | Hyper-methylated | PCa\|NT DMRs |
| chr2 | 175190675 | 175199882 | 2.15E−30 | 27.129634 | Hyper-methylated | PCa\|NT DMRs |
| chr2 | 175437120 | 175445027 | 0.004095673 | 26.68650794 | Hyper-methylated | PCa\|NT DMRs |
| chr2 | 177001780 | 177004583 | 4.70E−07 | 38.03968254 | Hyper-methylated | PCa\|NT DMRs |
| chr2 | 177053478 | 177054838 | 1.53E−20 | 26.16380952 | Hyper-methylated | PCa\|NT DMRs |
| chr2 | 179397807 | 179399188 | 0.001969641 | 46.63605442 | Hyper-methylated | PCa\|NT DMRs |
| chr2 | 180184481 | 180184526 | 0.005968871 | −25.92666667 | Hypo-methylated | PCa\|NT DMRs |
| chr2 | 180726644 | 180726742 | 0.000117699 | 44.8025974 | Hyper-methylated | PCa\|NT DMRs |
| chr2 | 183579384 | 183579582 | 0.00064571 | −48.63227513 | Hypo-methylated | PCa\|NT DMRs |
| chr2 | 184594781 | 184594866 | 0.001969641 | −43.47619048 | Hypo-methylated | PCa\|NT DMRs |
| chr2 | 184957584 | 185440919 | 7.58E−13 | −31.97978836 | Hypo-methylated | PCa\|NT DMRs |
| chr2 | 185463763 | 185602040 | 2.42E−05 | −34.81278195 | Hypo-methylated | PCa\|NT DMRs |
| chr2 | 185708150 | 185918405 | 0.001969641 | −23.43333333 | Hypo-methylated | PCa\|NT DMRs |
| chr2 | 186123428 | 186230566 | 0.00017871 | −20.22095238 | Hypo-methylated | PCa\|NT DMRs |
| chr2 | 187735461 | 187799347 | 0.004095673 | −28.1984127 | Hypo-methylated | PCa\|NT DMRs |
| chr2 | 187837330 | 187867958 | 3.65E−05 | −24.39724311 | Hypo-methylated | PCa\|NT DMRs |
| chr2 | 191042211 | 191045733 | 1.80E−22 | 37.05605787 | Hyper-methylated | PCa\|NT DMRs |
| chr2 | 192130371 | 192130815 | 0.001969641 | −46.33333333 | Hypo-methylated | PCa\|NT DMRs |
| chr2 | 197501365 | 197501514 | 0.00064571 | −34.76190476 | Hypo-methylated | PCa\|NT DMRs |
| chr2 | 198029845 | 198044986 | 1.97E−05 | −54.38333333 | Hypo-methylated | PCa\|NT DMRs |
| chr2 | 198455484 | 198455514 | 0.005968871 | 26.25714286 | Hyper-methylated | PCa\|NT DMRs |
| chr2 | 198670398 | 198670628 | 0.007959104 | 28.13877551 | Hyper-methy lated | PCa\|NT DMRs |
| chr2 | 203036152 | 203038067 | 0.001189237 | 32.95918367 | Hyper-methy lated | PCa\|NT DMRs |
| chr2 | 204975155 | 204975981 | 0.00064571 | 35.23129252 | Hyper-methylated | PCa\|NT DMRs |
| chr2 | 206551075 | 206551748 | 0.000216661 | 28.54235589 | Hyper-methy lated | PCa\|NT DMRs |
| chr2 | 208372808 | 208393580 | 0.001037738 | −35.16349206 | Hypo-methylated | PCa\|NT DMRs |
| chr2 | 209248665 | 209257588 | 0.001171269 | −21.49350649 | Hypo-methylated | PCa\|NT DMRs |
| chr2 | 211633104 | 211790226 | 0.002097193 | −30.6122449 | Hypo-methylated | PCa\|NT DMRs |
| chr2 | 216870971 | 216871374 | 0.00641359 | 39.62698413 | Hyper-methylated | PCa\|NT DMRs |
| chr2 | 217118607 | 217118739 | 0.005968871 | −44.12380952 | Hypo-methylated | PCa\|NT DMRs |
| chr2 | 217501703 | 217502200 | 0.00064571 | 28.28911565 | Hyper-methylated | PCa\|NT DMRs |
| chr2 | 218663904 | 218663979 | 0.005968871 | 49.86666667 | Hyper-methy lated | PCa\|NT DMRs |
| chr2 | 218809738 | 218845722 | 2.22E−05 | −32.93371429 | Hypo-methylated | PCa\|NT DMRs |
| chr2 | 218897690 | 218899361 | 0.009255851 | 29.27647059 | Hyper-methy lated | PCa\|NT DMRs |
| chr2 | 219192754 | 219203609 | 0.001509282 | 24.27919799 | Hyper-methy lated | PCa\|NT DMRs |
| chr2 | 219221846 | 219222123 | 0.003448944 | −32.59727891 | Hypo-methylated | PCa\|NT DMRs |
| chr2 | 219266966 | 219268668 | 2.43E−07 | −30.41380952 | Hypo-methylated | PCa\|NT DMRs |
| chr2 | 219646460 | 219647412 | 2.98E−16 | 43.11980952 | Hyper-methy lated | PCa\|NT DMRs |
| chr2 | 220024466 | 220024769 | 0.000165902 | −20.3040293 | Hypo-methy lated | PCa\|NT DMRs |
| chr2 | 220117660 | 220117987 | 1.37E−07 | 30.18047619 | Hyper-methylated | PCa\|NT DMRs |
| chr2 | 220306200 | 220306250 | 0.004095673 | 25.28571429 | Hyper-methy lated | PCa\|NT DMRs |
| chr2 | 220360817 | 220361690 | 2.04E−06 | 24.5037518 | Hyper-methylated | PCa\|NT DMRs |
| chr2 | 220414026 | 220416721 | 8.37E−05 | 25.59761905 | Hyper-methylated | PCa\|NT DMRs |
| chr2 | 220439769 | 220457830 | 0.001709403 | −24.09255952 | Hypo-methylated | PCa\|NT DMRs |
| chr2 | 220493909 | 220493959 | 0.005968871 | 63.50285714 | Hyper-methylated | PCa\|NT DMRs |
| chr2 | 224904321 | 224904735 | 2.70E−07 | 30.70666667 | Hyper-methylated | PCa\|NT DMRs |
| chr2 | 225266298 | 225266368 | 0.005968871 | 54.82857143 | Hyper-methylated | PCa\|NT DMRs |
| chr2 | 225639605 | 225639745 | 0.000967268 | 21.0952381 | Hyper-methylated | PCa\|NT DMRs |
| chr2 | 228028289 | 228028649 | 5.82E−05 | 26.39285714 | Hyper-methylated | PCa\|NT DMRs |
| chr2 | 229120368 | 229170764 | 3.66E−05 | −30.24047619 | Hypo-methylated | PCa\|NT DMRs |
| chr2 | 229326260 | 229879298 | 3.42E−13 | −25.45393586 | Hypo-methylated | PCa\|NT DMRs |
| chr2 | 230274961 | 230275101 | 0.005968871 | −24.68571429 | Hypo-methylated | PCa\|NT DMRs |
| chr2 | 231524469 | 231525078 | 0.005968871 | 53.37142857 | Hyper-methylated | PCa\|NT DMRs |
| chr2 | 231693254 | 231693446 | 0.000570487 | 20.78095238 | Hyper-methylated | PCa\|NT DMRs |
| chr2 | 231732670 | 231734772 | 0.005968871 | 21.78888889 | Hyper-methylated | PCa\|NT DMRs |
| chr2 | 231740522 | 231743113 | 0.000729802 | 22.38888889 | Hyper-methylated | PCa\|NT DMRs |
| chr2 | 231809233 | 231809291 | 0.001969641 | 22.42460317 | Hyper-methylated | PCa\|NT DMRs |
| chr2 | 231854776 | 231855941 | 2.25E−13 | 21.24337194 | Hyper-methylated | PCa\|NT DMRs |
| chr2 | 232055077 | 232055340 | 0.004231599 | −22.38974359 | Hypo-methylated | PCa\|NT DMRs |
| chr2 | 232056484 | 232062135 | 7.31E−05 | −22.88605442 | Hypo-methylated | PCa\|NT DMRs |
| chr2 | 232186761 | 232186899 | 0.001969641 | 53.82619048 | Hyper-methylated | PCa\|NT DMRs |
| chr2 | 232236067 | 232236424 | 0.000418089 | 23.56065163 | Hyper-methylated | PCa\|NT DMRs |
| chr2 | 232253187 | 232258762 | 0.008581782 | 31.26468254 | Hyper-methylated | PCa\|NT DMRs |
| chr2 | 232347993 | 232354900 | 4.96E−07 | 35.86691087 | Hyper-methylated | PCa\|NT DMRs |
| chr2 | 232425497 | 232428092 | 0.00641359 | −39.16269841 | Hypo-methylated | PCa\|NT DMRs |
| chr2 | 232479216 | 232480171 | 2.70E−14 | 37.56240079 | Hyper-methylated | PCa\|NT DMRs |
| chr2 | 233252274 | 233253378 | 4.72E−12 | −33.79213732 | Hypo-methylated | PCa\|NT DMRs |
| chr2 | 233285941 | 233286151 | 0.001083007 | 20.04047619 | Hyper-methy lated | PCa\|NT DMRs |
| chr2 | 233312595 | 233312629 | 0.003448944 | −21.06122449 | Hypo-methylated | PCa\|NT DMRs |
| chr2 | 233498313 | 233500251 | 9.84E−18 | 31.67488329 | Hyper-methy lated | PCa\|NT DMRs |
| chr2 | 233790639 | 233791818 | 0.008897139 | 24.495671 | Hyper-methylated | PCa\|NT DMRs |
| chr2 | 233809607 | 233815863 | 0.000205174 | −22.36428571 | Hypo-methy lated | PCa\|NT DMRs |
| chr2 | 233841555 | 233849331 | 6.04E−05 | −30.19350649 | Hypo-methy lated | PCa\|NT DMRs |
| chr2 | 233925165 | 233929137 | 3.34E−08 | 43.3358631 | Hyper-methylated | PCa\|NT DMRs |
| chr2 | 233943999 | 233950039 | 1.04E−05 | 26.41440186 | Hyper-methylated | PCa\|NT DMRs |
| chr2 | 234062066 | 234072770 | 0.000481535 | −23.85185185 | Hypo-methylated | PCa\|NT DMRs |
| chr2 | 234180219 | 234198585 | 0.00064571 | −44.50680272 | Hypo-methylated | PCa\|NT DMRs |
| chr2 | 234264046 | 234283708 | 0.009195794 | −22.14161491 | Hypo-methylated | PCa\|NT DMRs |
| chr2 | 234369848 | 234370089 | 4.45E−08 | −48.27428571 | Hypo-methylated | PCa\|NT DMRs |
| chr2 | 234389588 | 234389764 | 0.004095673 | −43.64285714 | Hypo-methylated | PCa\|NT DMRs |
| chr2 | 234756972 | 234757125 | 0.001969641 | 30.23809524 | Hyper-methylated | PCa\|NT DMRs |
| chr2 | 235539582 | 235541338 | 0.001969641 | −41.09444444 | Hypo-methylated | PCa\|NT DMRs |
| chr2 | 235709338 | 235709681 | 0.000205174 | −46.85535714 | Hypo-methylated | PCa\|NT DMRs |
| chr2 | 235906815 | 235916154 | 0.006225823 | −23.54285714 | Hypo-methylated | PCa\|NT DMRs |
| chr2 | 235917354 | 235917891 | 0.003622801 | −34.34126984 | Hypo-methylated | PCa\|NT DMRs |
| chr2 | 235936118 | 235936841 | 0.003056323 | −43.5968254 | Hypo-methylated | PCa\|NT DMRs |
| chr2 | 235961365 | 235961783 | 0.005968871 | −40.33015873 | Hypo-methylated | PCa\|NT DMRs |
| chr2 | 236474828 | 236475664 | 0.003391914 | 29.56878307 | Hyper-methylated | PCa\|NT DMRs |
| chr2 | 236961710 | 236963528 | 0.007840675 | 25.3358396 | Hyper-methylated | PCa\|NT DMRs |
| chr2 | 237077414 | 237085816 | 1.81E−43 | 31.65858319 | Hyper-methylated | PCa\|NT DMRs |
| chr2 | 237263701 | 237278472 | 0.001775064 | −42.91666667 | Hypo-methylated | PCa\|NT DMRs |
| chr2 | 237416056 | 237416123 | 0.000481535 | 21.63492063 | Hyper-methylated | PCa\|NT DMRs |
| chr2 | 237476476 | 237476892 | 1.25E−10 | 22.01142857 | Hyper-methylated | PCa\|NT DMRs |
| chr2 | 237685525 | 237685823 | 0.001969641 | −58.49206349 | Hypo-methylated | PCa\|NT DMRs |
| chr2 | 237712355 | 237712492 | 0.005968871 | 32.99428571 | Hyper-methylated | PCa\|NT DMRs |
| chr2 | 237969260 | 237970369 | 0.001969641 | −42.66111111 | Hypo-methylated | PCa\|NT DMRs |
| chr2 | 238187125 | 238207725 | 0.000260936 | −26.33952381 | Hypo-methylated | PCa\|NT DMRs |
| chr2 | 238261903 | 238341455 | 3.21E−22 | −21.85297619 | Hypo-methylated | PCa\|NT DMRs |
| chr2 | 238359571 | 238360255 | 1.97E−05 | −22.82047619 | Hypo-methylated | PCa\|NT DMRs |
| chr2 | 238420737 | 238433778 | 0.00032872 | −23.68888889 | Hypo-methylated | PCa\|NT DMRs |
| chr2 | 238642459 | 238644009 | 2.60E−05 | −39.44255952 | Hypo-methylated | PCa\|NT DMRs |
| chr2 | 238769342 | 238769420 | 0.004057568 | −50.64285714 | Hypo-methylated | PCa\|NT DMRs |
| chr2 | 239017454 | 239023305 | 0.000513415 | 23.95816327 | Hyper-methylated | PCa\|NT DMRs |
| chr2 | 239072275 | 239073970 | 4.02E−23 | 21.36383526 | Hyper-methylated | PCa\|NT DMRs |
| chr2 | 239435070 | 239437807 | 0.001969641 | −30.43650794 | Hypo-methylated | PCa\|NT DMRs |
| chr2 | 239694252 | 239705403 | 2.88E−05 | −24.79254658 | Hypo-methylated | PCa\|NT DMRs |
| chr2 | 239837692 | 239841275 | 4.63E−07 | −20.71785714 | Hypo-methylated | PCa\|NT DMRs |
| chr2 | 239981099 | 239981172 | 0.005968871 | 47.3952381 | Hyper-methylated | PCa\|NT DMRs |
| chr2 | 239997573 | 239997951 | 0.000377317 | −22.59702381 | Hypo-methylated | PCa\|NT DMRs |
| chr2 | 240076192 | 240076816 | 0.001160639 | 26.27142857 | Hyper-methylated | PCa\|NT DMRs |
| chr2 | 240079628 | 240079717 | 0.005968871 | −60.73333333 | Hypo-methylated | PCa\|NT DMRs |
| chr2 | 240167680 | 240168313 | 1.66E−10 | −42.70238095 | Hypo-methylated | PCa\|NT DMRs |
| chr2 | 240187878 | 240198162 | 0.006535327 | 24.36607143 | Hyper-methylated | PCa\|NT DMRs |
| chr2 | 240218300 | 240218487 | 0.00641359 | −28.45238095 | Hypo-methylated | PCa\|NT DMRs |
| chr2 | 240238759 | 240239010 | 0.001969641 | −47.6031746 | Hypo-methylated | PCa\|NT DMRs |
| chr2 | 240270058 | 240270798 | 0.001083007 | −35.08333333 | Hypo-methylated | PCa\|NT DMRs |
| chr2 | 240279807 | 240280163 | 0.001969641 | −53.08231293 | Hypo-methylated | PCa\|NT DMRs |
| chr2 | 240302429 | 240302645 | 0.00028029 | 34.85714286 | Hyper-methylated | PCa\|NT DMRs |
| chr2 | 240361406 | 240383441 | 0.000231303 | −24.56122449 | Hypo-methylated | PCa\|NT DMRs |
| chr2 | 240383511 | 240386085 | 1.95E−07 | −27.98412698 | Hypo-methylated | PCa\|NT DMRs |
| chr2 | 240399323 | 240410828 | 6.71E−09 | −22.314518 | Hypo-methylated | PCa\|NT DMRs |
| chr2 | 240420347 | 240421202 | 1.29E−06 | −33.869161 | Hypo-methylated | PCa\|NT DMRs |
| chr2 | 240430671 | 240451507 | 8.63E−13 | −25.98992063 | Hypo-methylated | PCa\|NT DMRs |
| chr2 | 240458193 | 240506049 | 6.60E−24 | −27.07705106 | Hypo-methylated | PCa\|NT DMRs |
| chr2 | 240515779 | 240518673 | 0.001344447 | −39.29107143 | Hypo-methylated | PCa\|NT DMRs |
| chr2 | 240530602 | 240532221 | 1.97E−05 | −31.06190476 | Hypo-methylated | PCa\|NT DMRs |
| chr2 | 240567882 | 240568112 | 0.005438982 | −26.56761905 | Hypo-methylated | PCa\|NT DMRs |
| chr2 | 240586327 | 240593374 | 3.44E−06 | −30.60714286 | Hypo-methylated | PCa\|NT DMRs |
| chr2 | 240599941 | 240619661 | 2.54E−07 | −23.92113095 | Hypo-methylated | PCa\|NT DMRs |
| chr2 | 240658654 | 240665479 | 7.52E−06 | −23.83756614 | Hypo-methylated | PCa\|NT DMRs |
| chr2 | 240672302 | 240672703 | 6.42E−05 | −32.11111111 | Hypo-methylated | PCa\|NT DMRs |
| chr2 | 240673060 | 240674119 | 2.52E−05 | 24.32773109 | Hyper-methylated | PCa\|NT DMRs |
| chr2 | 240689139 | 240689287 | 0.00064571 | −34.46122449 | Hypo-methylated | PCa\|NT DMRs |
| chr2 | 240698302 | 240731445 | 1.47E−16 | −28.12758621 | Hypo-methylated | PCa\|NT DMRs |
| chr2 | 240749506 | 240749872 | 0.005968871 | −41.74285714 | Hypo-methylated | PCa\|NT DMRs |
| chr2 | 240985852 | 241010928 | 0.00013778 | −27.13003663 | Hypo-methylated | PCa\|NT DMRs |
| chr2 | 241190512 | 241190982 | 0.000695921 | −34.98746867 | Hypo-methylated | PCa\|NT DMRs |
| chr2 | 241287924 | 241293252 | 6.77E−05 | −21.2873706 | Hypo-methylated | PCa\|NT DMRs |
| chr2 | 241303199 | 241304062 | 0.000564835 | −27.1 | Hypo-methylated | PCa\|NT DMRs |
| chr2 | 241317552 | 241323074 | 2.86E−05 | −22.61538462 | Hypo-methylated | PCa\|NT DMRs |
| chr2 | 241333368 | 241337189 | 0.000215808 | −31.76984127 | Hypo-methylated | PCa\|NT DMRs |
| chr2 | 241387484 | 241387957 | 0.000365371 | −23.93613445 | Hypo-methylated | PCa\|NT DMRs |
| chr2 | 241562086 | 241564964 | 1.09E−10 | 42.56785714 | Hyper-methylated | PCa\|NT DMRs |
| chr2 | 241775840 | 241795756 | 0.000614706 | −22.58116883 | Hypo-methylated | PCa\|NT DMRs |
| chr2 | 241853025 | 241854588 | 6.69E−05 | −49.19761905 | Hypo-methylated | PCa\|NT DMRs |
| chr2 | 241855280 | 241856157 | 0.00034072 | 26.78151261 | Hyper-methylated | PCa\|NT DMRs |
| chr2 | 241904140 | 241905740 | 1.52E−06 | 30.23870968 | Hyper-methylated | PCa\|NT DMRs |
| chr2 | 241930909 | 241931550 | 3.81E−05 | −42.8012987 | Hypo-methylated | PCa\|NT DMRs |
| chr2 | 242031375 | 242034103 | 0.001083007 | −29.84190476 | Hypo-methylated | PCa\|NT DMRs |
| chr2 | 242063157 | 242065890 | 0.00128874 | −41.05194805 | Hypo-methylated | PCa\|NT DMRs |
| chr2 | 242127948 | 242128930 | 8.23E−07 | −34.48819048 | Hypo-methylated | PCa\|NT DMRs |
| chr2 | 242139786 | 242141146 | 1.45E−07 | 25.54489796 | Hyper-methylated | PCa\|NT DMRs |
| chr2 | 242195217 | 242209595 | 0.000847246 | −28.44659864 | Hypo-methylated | PCa\|NT DMRs |
| chr2 | 242448612 | 242448879 | 0.000215808 | 22.02116402 | Hyper-methylated | PCa\|NT DMRs |
| chr2 | 242480249 | 242480505 | 3.36E−06 | 22.03726708 | Hyper-methylated | PCa\|NT DMRs |
| chr2 | 242489719 | 242490335 | 0.001789867 | −36.03968254 | Hypo-methylated | PCa\|NT DMRs |
| chr2 | 242602177 | 242605056 | 8.39E−05 | −24.7605042 | Hypo-methylated | PCa\|NT DMRs |
| chr2 | 242702750 | 242706889 | 0.000166226 | 33.25824176 | Hyper-methylated | PCa\|NT DMRs |
| chr2 | 242713019 | 242713689 | 0.004162937 | −32.56904762 | Hypo-methylated | PCa\|NT DMRs |
| chr2 | 242713751 | 242713888 | 0.002534928 | 28.53809524 | Hyper-methylated | PCa\|NT DMRs |
| chr2 | 242743649 | 242744590 | 1.66E−07 | 29.56568144 | Hyper-methylated | PCa\|NT DMRs |
| chr2 | 242763866 | 242764069 | 0.005295761 | 28.37662338 | Hyper-methylated | PCa\|NT DMRs |
| chr2 | 242774275 | 242776222 | 0.004231599 | −20.69267399 | Hypo-methylated | PCa\|NT DMRs |
| chr2 | 242784847 | 242786128 | 7.01E−09 | 41.80367347 | Hyper-methylated | PCa\|NT DMRs |
| chr2 | 242794855 | 242794981 | 1.69E−06 | −26.48711485 | Hypo-methylated | PCa\|NT DMRs |
| chr2 | 242805857 | 242810434 | 0.000104747 | 23.99085213 | Hyper-methylated | PCa\|NT DMRs |
| chr2 | 242829929 | 242834690 | 4.29E−06 | −40.41981982 | Hypo-methylated | PCa\|NT DMRs |
| chr2 | 242843822 | 242844721 | 6.54E−11 | 21.08558559 | Hyper-methylated | PCa\|NT DMRs |
| chr2 | 242927565 | 242927898 | 2.11E−06 | 36.92559524 | Hyper-methylated | PCa\|NT DMRs |
| chr2 | 242946990 | 242947347 | 0.000209339 | −33.80952381 | Hypo-methylated | PCa\|NT DMRs |
| chr3 | 2189939 | 2192851 | 0.00064571 | −30.58503401 | Hypo-methylated | PCa\|NT DMRs |
| chr3 | 3840551 | 3840913 | 5.41E−09 | 24.50095238 | Hyper-methylated | PCa\|NT DMRs |
| chr3 | 4345887 | 4345919 | 0.005968871 | −32.64 | Hypo-methylated | PCa\|NT DMRs |
| chr3 | 5141437 | 5155620 | 0.001789867 | −23.59259259 | Hypo-methylated | PCa\|NT DMRs |
| chr3 | 6654049 | 6715713 | 0.001969641 | −30.27698413 | Hypo-methylated | PCa\|NT DMRs |
| chr3 | 9222564 | 9222873 | 0.005968871 | 59.79428571 | Hyper-methylated | PCa\|NT DMRs |
| chr3 | 9771218 | 9771280 | 0.001969641 | −31.57142857 | Hypo-methylated | PCa\|NT DMRs |
| chr3 | 9884796 | 9884931 | 0.000118316 | −22.51785714 | Hypo-methylated | PCa\|NT DMRs |
| chr3 | 9987430 | 9996973 | 6.59E−11 | −28.9437996 | Hypo-methylated | PCa\|NT DMRs |
| chr3 | 10206210 | 10206259 | 0.003622801 | −25.16666667 | Hypo-methylated | PCa\|NT DMRs |
| chr3 | 10325249 | 10325495 | 0.005968871 | −37.36190476 | Hypo-methylated | PCa\|NT DMRs |
| chr3 | 10352032 | 10354338 | 1.43E−06 | −27.50793651 | Hypo-methylated | PCa\|NT DMRs |
| chr3 | 10370439 | 10371665 | 0.000111427 | −28.26904762 | Hypo-methylated | PCa\|NT DMRs |
| chr3 | 10651088 | 10662428 | 0.001013662 | −20.91666667 | Hypo-methylated | PCa\|NT DMRs |
| chr3 | 10805784 | 10806395 | 0.000109295 | 22.54458874 | Hyper-methylated | PCa\|NT DMRs |
| chr3 | 10987978 | 10992387 | 0.007628253 | −31.56462585 | Hypo-methylated | PCa\|NT DMRs |
| chr3 | 11035187 | 11035383 | 7.88E−06 | 22.11160714 | Hyper-methylated | PCa\|NT DMRs |
| chr3 | 11102832 | 11103118 | 0.0005791 | −27.73669468 | Hypo-methylated | PCa\|NT DMRs |
| chr3 | 11267851 | 11284261 | 0.000377317 | −22.63214286 | Hypo-methylated | PCa\|NT DMRs |
| chr3 | 11303244 | 11309033 | 0.005968871 | −34.23714286 | Hypo-methylated | PCa\|NT DMRs |
| chr3 | 12111766 | 12112029 | 0.001969641 | −30.08730159 | Hypo-methylated | PCa\|NT DMRs |
| chr3 | 12586068 | 12592851 | 0.001969641 | −33.34285714 | Hypo-methylated | PCa\|NT DMRs |
| chr3 | 12966141 | 12966358 | 0.00179283 | 27.11428571 | Hyper-methylated | PCa\|NT DMRs |
| chr3 | 13082454 | 13082682 | 0.005513661 | 32.42517007 | Hyper-methylated | PCa\|NT DMRs |
| chr3 | 13107889 | 13107931 | 0.001969641 | 38.4047619 | Hyper-methylated | PCa\|NT DMRs |
| chr3 | 13134381 | 13134439 | 0.00064571 | −43.2244898 | Hypo-methylated | PCa\|NT DMRs |
| chr3 | 13153914 | 13153995 | 0.005968871 | −54.6 | Hypo-methylated | PCa\|NT DMRs |
| chr3 | 13163633 | 13164899 | 0.008581782 | −22.26785714 | Hypo-methylated | PCa\|NT DMRs |
| chr3 | 13261052 | 13261521 | 3.87E−05 | −29.15686275 | Hypo-methylated | PCa\|NT DMRs |
| chr3 | 13295875 | 13296368 | 0.001789867 | 21.97089947 | Hyper-methylated | PCa\|NT DMRs |
| chr3 | 13322755 | 13324142 | 1.35E−12 | 29.0487619 | Hyper-methylated | PCa\|NT DMRs |
| chr3 | 13453556 | 13454895 | 0.005968871 | −29.97142857 | Hypo-methylated | PCa\|NT DMRs |
| chr3 | 13456129 | 13460374 | 0.000172237 | 36.15136054 | Hyper-methylated | PCa\|NT DMRs |
| chr3 | 13535801 | 13536396 | 0.003622801 | −24.82142857 | Hypo-methylated | PCa\|NT DMRs |
| chr3 | 13590903 | 13595543 | 0.00068166 | 21.76329365 | Hyper-methylated | PCa\|NT DMRs |
| chr3 | 13634770 | 13648506 | 0.000145812 | −25.45684524 | Hypo-methylated | PCa\|NT DMRs |
| chr3 | 13696498 | 13696590 | 0.001115302 | 31.79285714 | Hyper-methylated | PCa\|NT DMRs |
| chr3 | 13886422 | 13906816 | 6.73E−09 | −29.0985843 | Hypo-methylated | PCa\|NT DMRs |
| chr3 | 14347416 | 14349701 | 0.002767976 | −26.77359307 | Hypo-methylated | PCa\|NT DMRs |
| chr3 | 14351112 | 14359347 | 0.000840726 | 24.59365079 | Hyper-methylated | PCa\|NT DMRs |
| chr3 | 14494726 | 14494916 | 0.001969641 | −59.59183673 | Hypo-methylated | PCa\|NT DMRs |
| chr3 | 14597401 | 14597677 | 2.14E−08 | 48.06948854 | Hyper-methylated | PCa\|NT DMRs |
| chr3 | 14852520 | 14852812 | 9.95E−11 | 20.65414966 | Hyper-methylated | PCa\|NT DMRs |
| chr3 | 14944084 | 14962565 | 0.000440405 | −27.13157895 | Hypo-methylated | PCa\|NT DMRs |
| chr3 | 16359535 | 16359699 | 0.005968871 | 51.98571429 | Hyper-methylated | PCa\|NT DMRs |
| chr3 | 16572713 | 16625310 | 6.90E−06 | −31.86904762 | Hypo-methylated | PCa\|NT DMRs |
| chr3 | 16661170 | 16811682 | 5.94E−06 | −42.17316017 | Hypo-methylated | PCa\|NT DMRs |
| chr3 | 19777033 | 19870137 | 4.90E−08 | −27.9534632 | Hypo-methylated | PCa\|NT DMRs |
| chr3 | 20409494 | 20475035 | 1.03E−05 | −34.93095238 | Hypo-methylated | PCa\|NT DMRs |
| chr3 | 21095717 | 21267469 | 5.53E−08 | −31.78181818 | Hypo-methylated | PCa\|NT DMRs |
| chr3 | 22458288 | 22868648 | 2.49E−12 | −36.17619048 | Hypo-methylated | PCa\|NT DMRs |
| chr3 | 23784649 | 23785077 | 4.90E−08 | −45.83441558 | Hypo-methylated | PCa\|NT DMRs |
| chr3 | 24563543 | 24563856 | 0.000196301 | 33.91666667 | Hyper-methylated | PCa\|NT DMRs |
| chr3 | 25788413 | 25822319 | 0.001391421 | 24.56623377 | Hyper-methylated | PCa\|NT DMRs |
| chr3 | 27830321 | 27857016 | 0.003448944 | −26.16258503 | Hypo-methylated | PCa\|NT DMRs |
| chr3 | 32324971 | 32325053 | 0.005968871 | 28.54285714 | Hyper-methylated | PCa\|NT DMRs |
| chr3 | 32542453 | 32542607 | 0.002534928 | 37.9 | Hyper-methylated | PCa\|NT DMRs |
| chr3 | 33806130 | 33819158 | 0.002097193 | −38.67006803 | Hypo-methylated | PCa\|NT DMRs |
| chr3 | 33923352 | 33997649 | 0.000392715 | −21.95047619 | Hypo-methylated | PCa\|NT DMRs |
| chr3 | 35749002 | 36384340 | 6.29E−12 | −29.0510989 | Hypo-methylated | PCa\|NT DMRs |
| chr3 | 36433155 | 36581379 | 5.52E−06 | −23.97117794 | Hypo-methylated | PCa\|NT DMRs |
| chr3 | 36805618 | 36805655 | 0.001969641 | 30.5 | Hyper-methylated | PCa\|NT DMRs |
| chr3 | 36873851 | 36874173 | 0.001451132 | −22.87301587 | Hypo-methylated | PCa\|NT DMRs |
| chr3 | 38022164 | 38036310 | 8.69E−06 | 21.08294574 | Hyper-methylated | PCa\|NT DMRs |
| chr3 | 38080673 | 38081695 | 1.94E−17 | 27.74231806 | Hyper-methylated | PCa\|NT DMRs |
| chr3 | 38157991 | 38158048 | 0.005968871 | −43.20952381 | Hypo-methylated | PCa\|NT DMRs |
| chr3 | 38698863 | 38768663 | 5.42E−07 | −40.19312169 | Hypo-methylated | PCa\|NT DMRs |
| chr3 | 38793972 | 38980352 | 3.13E−09 | −37.34479718 | Hypo-methylated | PCa\|NT DMRs |
| chr3 | 39184553 | 39185795 | 7.95E−05 | −35.25824176 | Hypo-methylated | PCa\|NT DMRs |
| chr3 | 40368753 | 40368789 | 0.005968871 | −37.45714286 | Hypo-methylated | PCa\|NT DMRs |
| chr3 | 40656796 | 40657331 | 9.46E−05 | 31.07969925 | Hyper-methylated | PCa\|NT DMRs |
| chr3 | 40821888 | 40822906 | 0.001115302 | −39.56547619 | Hypo-methylated | PCa\|NT DMRs |
| chr3 | 42005345 | 42008186 | 0.005513661 | −21.17857143 | Hypo-methylated | PCa\|NT DMRs |
| chr3 | 42568067 | 42568556 | 0.000294557 | 24.74199134 | Hyper-methylated | PCa\|NT DMRs |
| chr3 | 42586344 | 42586708 | 0.00023371 | −58.84285714 | Hypo-methylated | PCa\|NT DMRs |
| chr3 | 42740568 | 42740650 | 0.005968871 | 41.71428571 | Hyper-methylated | PCa\|NT DMRs |
| chr3 | 42947590 | 42947903 | 1.02E−12 | 24.24350064 | Hyper-methylated | PCa\|NT DMRs |
| chr3 | 43037037 | 43049564 | 0.002437742 | −26.65925926 | Hypo-methylated | PCa\|NT DMRs |
| chr3 | 43830160 | 43862874 | 0.00179283 | −20.26785714 | Hypo-methylated | PCa\|NT DMRs |
| chr3 | 44069059 | 44075998 | 0.001969641 | −22.5 | Hypo-methylated | PCa\|NT DMRs |
| chr3 | 45821493 | 45821693 | 0.005513661 | −26.69047619 | Hypo-methylated | PCa\|NT DMRs |
| chr3 | 46009288 | 46009664 | 0.001969641 | −47.07142857 | Hypo-methylated | PCa\|NT DMRs |
| chr3 | 46607381 | 46607468 | 0.008482405 | 29.8 | Hyper-methylated | PCa\|NT DMRs |
| chr3 | 46735437 | 46735616 | 0.009069241 | 28.57142857 | Hyper-methylated | PCa\|NT DMRs |
| chr3 | 46853817 | 46853972 | 0.002243506 | 21.71212121 | Hyper-methylated | PCa\|NT DMRs |
| chr3 | 47029398 | 47029597 | 0.000205174 | 44.7952381 | Hyper-methylated | PCa\|NT DMRs |
| chr3 | 48131229 | 48131298 | 0.002097193 | −30.12244898 | Hypo-methylated | PCa\|NT DMRs |
| chr3 | 48283597 | 48283898 | 0.007560755 | −29.04166667 | Hypo-methylated | PCa\|NT DMRs |
| chr3 | 48598501 | 48598978 | 0.001969641 | 35.64285714 | Hyper-methylated | PCa\|NT DMRs |
| chr3 | 48632250 | 48633317 | 7.15E−06 | 29.76642857 | Hyper-methylated | PCa\|NT DMRs |
| chr3 | 48636277 | 48636596 | 0.000305995 | −39.5 | Hypo-methylated | PCa\|NT DMRs |
| chr3 | 48746476 | 48750172 | 0.005438982 | −23.94285714 | Hypo-methylated | PCa\|NT DMRs |
| chr3 | 48883897 | 48884081 | 0.000377317 | −24.51845238 | Hypo-methylated | PCa\|NT DMRs |
| chr3 | 49705312 | 49705658 | 0.000157125 | −27.31809524 | Hypo-methylated | PCa\|NT DMRs |
| chr3 | 50311119 | 50314337 | 1.66E−25 | 37.18675856 | Hyper-methylated | PCa\|NT DMRs |
| chr3 | 50567634 | 50568126 | 1.91E−08 | 21.67676768 | Hyper-methylated | PCa\|NT DMRs |
| chr3 | 50625996 | 50637484 | 0.006410012 | 30.75288221 | Hyper-methylated | PCa\|NT DMRs |
| chr3 | 50643197 | 50645354 | 1.00E−05 | 25.99174603 | Hyper-methylated | PCa\|NT DMRs |
| chr3 | 51671285 | 51671477 | 0.003448944 | −21.23260073 | Hypo-methylated | PCa\|NT DMRs |
| chr3 | 52224975 | 52227349 | 0.000168936 | −47.19264069 | Hypo-methylated | PCa\|NT DMRs |
| chr3 | 52351836 | 52351956 | 3.43E−06 | 42.54464286 | Hyper-methylated | PCa\|NT DMRs |
| chr3 | 52472027 | 52472119 | 0.001969641 | −48.13605442 | Hypo-methylated | PCa\|NT DMRs |
| chr3 | 52500911 | 52505893 | 0.000243521 | −39.60779221 | Hypo-methylated | PCa\|NT DMRs |
| chr3 | 52524840 | 52525480 | 0.000266785 | 25.67962963 | Hyper-methylated | PCa\|NT DMRs |
| chr3 | 52529132 | 52529760 | 8.76E−09 | 36.61581633 | Hyper-methylated | PCa\|NT DMRs |
| chr3 | 52565909 | 52567260 | 0.001289384 | 24.57635468 | Hyper-methylated | PCa\|NT DMRs |
| chr3 | 52568571 | 52568729 | 0.00023371 | −20.18571429 | Hypo-methylated | PCa\|NT DMRs |
| chr3 | 53080649 | 53081278 | 9.01E−08 | 22.78354978 | Hyper-methylated | PCa\|NT DMRs |
| chr3 | 53709966 | 53740155 | 0.00248539 | 26.5452381 | Hyper-methylated | PCa\|NT DMRs |
| chr3 | 54157600 | 54157745 | 0.002463399 | 20.32467532 | Hyper-methylated | PCa\|NT DMRs |
| chr3 | 54597851 | 54597987 | 0.007628253 | −39.92517007 | Hypo-methylated | PCa\|NT DMRs |
| chr3 | 56135628 | 56135965 | 0.005968871 | 37.08095238 | Hyper-methylated | PCa\|NT DMRs |
| chr3 | 56717505 | 56717770 | 0.000443725 | −27.21496599 | Hypo-methylated | PCa\|NT DMRs |
| chr3 | 57945651 | 57945743 | 0.005968871 | 34.22857143 | Hyper-methylated | PCa\|NT DMRs |
| chr3 | 57996569 | 58006904 | 0.000205174 | −52.71428571 | Hypo-methylated | PCa\|NT DMRs |
| chr3 | 58107099 | 58107183 | 0.001969641 | −27.28571429 | Hypo-methylated | PCa\|NT DMRs |
| chr3 | 58163925 | 58163998 | 0.009787745 | 29.19428571 | Hyper-methylated | PCa\|NT DMRs |
| chr3 | 58572635 | 58572808 | 6.29E−09 | 33.29395604 | Hyper-methylated | PCa\|NT DMRs |
| chr3 | 58645085 | 58645403 | 0.004376829 | −45.65608466 | Hypo-methylated | PCa\|NT DMRs |
| chr3 | 61549137 | 61549449 | 4.09E−07 | 38.28320802 | Hyper-methylated | PCa\|NT DMRs |
| chr3 | 63899054 | 63899382 | 0.000696555 | −23.26017316 | Hypo-methylated | PCa\|NT DMRs |
| chr3 | 64210992 | 64253866 | 4.83E−09 | 36.58131868 | Hyper-methylated | PCa\|NT DMRs |
| chr3 | 65317166 | 65356001 | 4.35E−13 | 38.25769231 | Hyper-methylated | PCa\|NT DMRs |
| chr3 | 66452406 | 66453560 | 0.000118733 | −31.40687831 | Hypo-methylated | PCa\|NT DMRs |
| chr3 | 66831353 | 66939121 | 0.000136892 | 26.63238095 | Hyper-methylated | PCa\|NT DMRs |
| chr3 | 67706348 | 67706568 | 5.84E−05 | 58.26587302 | Hyper-methylated | PCa\|NT DMRs |
| chr3 | 69061575 | 69061831 | 0.005968871 | −33.2 | Hypo-methylated | PCa\|NT DMRs |
| chr3 | 69230383 | 69251212 | 0.000826653 | −32.39166667 | Hypo-methylated | PCa\|NT DMRs |
| chr3 | 71410847 | 71411051 | 0.001969641 | −34.5952381 | Hypo-methylated | PCa\|NT DMRs |
| chr3 | 71981727 | 72033362 | 0.00641359 | −29.58333333 | Hypo-methylated | PCa\|NT DMRs |
| chr3 | 72202300 | 72211631 | 0.004947537 | −38.76455026 | Hypo-methylated | PCa\|NT DMRs |
| chr3 | 72788015 | 72788226 | 3.91E−07 | 32.92205514 | Hyper-methylated | PCa\|NT DMRs |
| chr3 | 73563981 | 73570326 | 0.005968871 | −50.0952381 | Hypo-methylated | PCa\|NT DMRs |
| chr3 | 74000093 | 74080135 | 1.10E−05 | −20.56164021 | Hypo-methylated | PCa\|NT DMRs |
| chr3 | 75191507 | 75263736 | 6.42E−05 | −28.83862434 | Hypo-methylated | PCa\|NT DMRs |
| chr3 | 75376761 | 75412696 | 9.09E−06 | −29.89071429 | Hypo-methylated | PCa\|NT DMRs |
| chr3 | 75495819 | 75556133 | 4.29E−08 | −22.72721088 | Hypo-methylated | PCa\|NT DMRs |
| chr3 | 75861114 | 75871514 | 0.001969641 | −34.51984127 | Hypo-methylated | PCa\|NT DMRs |
| chr3 | 79188728 | 79333142 | 4.20E−07 | −20.44679089 | Hypo-methylated | PCa\|NT DMRs |
| chr3 | 79556294 | 79708316 | 1.76E−06 | −27.47063492 | Hypo-methylated | PCa\|NT DMRs |
| chr3 | 80394942 | 80479627 | 6.94E−06 | −27.56031746 | Hypo-methylated | PCa\|NT DMRs |
| chr3 | 80931665 | 81228753 | 4.69E−12 | −32.26353078 | Hypo-methylated | PCa\|NT DMRs |
| chr3 | 81312941 | 81525412 | 1.48E−07 | −27.5995671 | Hypo-methylated | PCa\|NT DMRs |
| chr3 | 81816604 | 81881850 | 1.00E−08 | −23.95805861 | Hypo-methylated | PCa\|NT DMRs |
| chr3 | 82160140 | 82555783 | 1.07E−10 | −30.06040816 | Hypo-methylated | PCa\|NT DMRs |
| chr3 | 82690498 | 82923743 | 1.04E−12 | −31.06679537 | Hypo-methylated | PCa\|NT DMRs |
| chr3 | 83324209 | 84144425 | 4.72E−16 | −34.1669088 | Hypo-methylated | PCa\|NT DMRs |
| chr3 | 84174379 | 84549910 | 4.21E−14 | −32.90931677 | Hypo-methylated | PCa\|NT DMRs |
| chr3 | 84597769 | 84622970 | 0.008482405 | −34.69714286 | Hypo-methylated | PCa\|NT DMRs |
| chr3 | 87138203 | 87138521 | 1.52E−08 | 43.65520282 | Hyper-methylated | PCa\|NT DMRs |
| chr3 | 88244957 | 89113736 | 2.92E−14 | −32.15160173 | Hypo-methylated | PCa\|NT DMRs |
| chr3 | 93882885 | 93897493 | 0.004095673 | −52.37222222 | Hypo-methylated | PCa\|NT DMRs |
| chr3 | 97770314 | 98030112 | 1.87E−05 | −27.21428571 | Hypo-methylated | PCa\|NT DMRs |
| chr3 | 98613529 | 98613594 | 0.005968871 | −29.28571429 | Hypo-methylated | PCa\|NT DMRs |
| chr3 | 99381532 | 99419185 | 0.001171269 | −27.82207792 | Hypo-methylated | PCa\|NT DMRs |
| chr3 | 100526581 | 100658522 | 6.42E−05 | −40.7962963 | Hypo-methylated | PCa\|NT DMRs |
| chr3 | 101396737 | 101402717 | 3.16E−07 | −42.93469388 | Hypo-methylated | PCa\|NT DMRs |
| chr3 | 102642298 | 102678691 | 0.000339087 | −24.6957672 | Hypo-methylated | PCa\|NT DMRs |
| chr3 | 103012414 | 103749921 | 2.94E−14 | −31.15238095 | Hypo-methylated | PCa\|NT DMRs |
| chr3 | 107675880 | 107712051 | 0.001430886 | −33.03571429 | Hypo-methylated | PCa\|NT DMRs |
| chr3 | 108434212 | 108599282 | 1.41E−05 | −28.54455782 | Hypo-methylated | PCa\|NT DMRs |
| chr3 | 108616526 | 108735432 | 3.44E−06 | −35.72142857 | Hypo-methylated | PCa\|NT DMRs |
| chr3 | 108835484 | 109054925 | 1.61E−24 | −28.07724868 | Hypo-methylated | PCa\|NT DMRs |
| chr3 | 109059738 | 109068430 | 0.000118733 | −26.98677249 | Hypo-methylated | PCa\|NT DMRs |
| chr3 | 109080333 | 109110314 | 0.001344447 | −39.64702381 | Hypo-methylated | PCa\|NT DMRs |
| chr3 | 109129857 | 109404368 | 2.86E−09 | −30.63218391 | Hypo-methylated | PCa\|NT DMRs |
| chr3 | 111410981 | 111411091 | 5.52E−05 | −33.09307359 | Hypo-methylated | PCa\|NT DMRs |
| chr3 | 112052124 | 112053518 | 4.56E−10 | 28.44162826 | Hyper-methylated | PCa\|NT DMRs |
| chr3 | 112357212 | 112510113 | 9.64E−14 | −39.12590012 | Hypo-methylated | PCa\|NT DMRs |
| chr3 | 112936919 | 112943015 | 0.005513661 | 29.30952381 | Hyper-methylated | PCa\|NT DMRs |
| chr3 | 113160075 | 113160706 | 5.14E−08 | 26.68973214 | Hyper-methylated | PCa\|NT DMRs |
| chr3 | 116742526 | 116742590 | 0.005968871 | 40.64761905 | Hyper-methylated | PCa\|NT DMRs |
| chr3 | 116990369 | 117193182 | 0.001969641 | −35.84603175 | Hypo-methylated | PCa\|NT DMRs |
| chr3 | 117733238 | 118730168 | 5.46E−37 | −31.16159251 | Hypo-methylated | PCa\|NT DMRs |
| chr3 | 118791508 | 118858083 | 6.84E−10 | −25.63065476 | Hypo-methylated | PCa\|NT DMRs |
| chr3 | 119697546 | 119697780 | 0.007679542 | −25.56243386 | Hypo-methylated | PCa\|NT DMRs |
| chr3 | 120157815 | 120157995 | 0.001969641 | −56.78571429 | Hypo-methylated | PCa\|NT DMRs |
| chr3 | 120170040 | 120170425 | 2.04E−05 | 22.77800454 | Hyper-methylated | PCa\|NT DMRs |
| chr3 | 122234462 | 122234498 | 0.005968871 | −36.07619048 | Hypo-methylated | PCa\|NT DMRs |
| chr3 | 123432259 | 123439101 | 1.97E−05 | −43.48809524 | Hypo-methylated | PCa\|NT DMRs |
| chr3 | 124781735 | 124782070 | 0.007233677 | −40.85416667 | Hypo-methylated | PCa\|NT DMRs |
| chr3 | 125123729 | 125123874 | 0.005968871 | −22.88571429 | Hypo-methylated | PCa\|NT DMRs |
| chr3 | 125480002 | 125482262 | 0.007338757 | 21.17857143 | Hyper-methylated | PCa\|NT DMRs |
| chr3 | 125898944 | 125900066 | 3.76E−21 | 21.58427318 | Hyper-methylated | PCa\|NT DMRs |
| chr3 | 125989071 | 125989239 | 0.005968871 | −55.82857143 | Hypo-methylated | PCa\|NT DMRs |
| chr3 | 126135364 | 126135401 | 0.008482405 | 44.07904762 | Hyper-methylated | PCa\|NT DMRs |
| chr3 | 126248069 | 126248458 | 0.004095673 | 41.41269841 | Hyper-methylated | PCa\|NT DMRs |
| chr3 | 126260408 | 126261355 | 9.01E−14 | −22.4850686 | Hypo-methylated | PCa\|NT DMRs |
| chr3 | 126381917 | 126381952 | 0.007560755 | −41.79464286 | Hypo-methylated | PCa\|NT DMRs |
| chr3 | 126860805 | 126861412 | 0.001969641 | −40.46031746 | Hypo-methylated | PCa\|NT DMRs |
| chr3 | 127069800 | 127069877 | 0.002826041 | 27.25034014 | Hyper-methylated | PCa\|NT DMRs |
| chr3 | 127175083 | 127175137 | 0.00064571 | 22.21164021 | Hyper-methylated | PCa\|NT DMRs |
| chr3 | 127266365 | 127266500 | 0.000363161 | 25.38392857 | Hyper-methylated | PCa\|NT DMRs |
| chr3 | 127439424 | 127441768 | 2.05E−05 | −27.01082251 | Hypo-methylated | PCa\|NT DMRs |
| chr3 | 127633608 | 127633888 | 0.001083007 | −40.62142857 | Hypo-methylated | PCa\|NT DMRs |
| chr3 | 127634527 | 127634587 | 0.001419801 | 27.93888889 | Hyper-methylated | PCa\|NT DMRs |
| chr3 | 127634788 | 127693494 | 0.001969641 | −22.8531746 | Hypo-methylated | PCa\|NT DMRs |
| chr3 | 127954900 | 127957492 | 0.005513661 | 21.44642857 | Hyper-methylated | PCa\|NT DMRs |
| chr3 | 128001142 | 128001759 | 1.76E−06 | −56.41349206 | Hypo-methylated | PCa\|NT DMRs |
| chr3 | 128123108 | 128123259 | 0.000974795 | −27.67857143 | Hypo-methylated | PCa\|NT DMRs |
| chr3 | 128274726 | 128276076 | 0.008109221 | 39.27142857 | Hyper-methylated | PCa\|NT DMRs |
| chr3 | 128294897 | 128295135 | 0.001969641 | −22.06746032 | Hypo-methylated | PCa\|NT DMRs |
| chr3 | 128307104 | 128326680 | 2.58E−05 | 30.41415701 | Hyper-methylated | PCa\|NT DMRs |
| chr3 | 128370457 | 128371298 | 0.005295761 | 29.63419913 | Hyper-methylated | PCa\|NT DMRs |
| chr3 | 128567687 | 128568612 | 0.002097193 | −30.37687075 | Hypo-methylated | PCa\|NT DMRs |
| chr3 | 128710421 | 128710530 | 0.001969641 | 51.20873016 | Hyper-methylated | PCa\|NT DMRs |
| chr3 | 129295896 | 129297909 | 0.000754912 | 44.79714286 | Hyper-methylated | PCa\|NT DMRs |
| chr3 | 129337801 | 129338205 | 1.66E−05 | 38.16246499 | Hyper-methylated | PCa\|NT DMRs |
| chr3 | 129342553 | 129342702 | 1.27E−08 | −61.62291667 | Hypo-methylated | PCa\|NT DMRs |
| chr3 | 132624879 | 132728974 | 7.57E−05 | −26.79722222 | Hypo-methylated | PCa\|NT DMRs |
| chr3 | 132922215 | 133099864 | 0.000648693 | −25.03968254 | Hypo-methylated | PCa\|NT DMRs |
| chr3 | 133291052 | 133291116 | 0.005968871 | 42.4 | Hyper-methylated | PCa\|NT DMRs |
| chr3 | 133464950 | 133465387 | 1.34E−12 | 31.22015504 | Hyper-methylated | PCa\|NT DMRs |
| chr3 | 133661523 | 133661586 | 0.008482405 | 57.62857143 | Hyper-methylated | PCa\|NT DMRs |
| chr3 | 134077285 | 134089922 | 4.20E−07 | 34.46696429 | Hyper-methylated | PCa\|NT DMRs |
| chr3 | 134378765 | 134513435 | 4.79E−08 | −28.46850998 | Hypo-methylated | PCa\|NT DMRs |
| chr3 | 134523940 | 135582304 | 2.59E−28 | −28.69820566 | Hypo-methylated | PCa\|NT DMRs |
| chr3 | 137487843 | 137490651 | 4.97E−30 | 21.44655522 | Hyper-methylated | PCa\|NT DMRs |
| chr3 | 137920396 | 137920465 | 0.00064571 | −25.71428571 | Hypo-methylated | PCa\|NT DMRs |
| chr3 | 138154011 | 138154738 | 2.06E−08 | 27.51972789 | Hyper-methylated | PCa\|NT DMRs |
| chr3 | 138608383 | 138628551 | 0.001460345 | 20.91904762 | Hyper-methylated | PCa\|NT DMRs |
| chr3 | 138654723 | 138654889 | 0.003622801 | 36.02380952 | Hyper-methylated | PCa\|NT DMRs |
| chr3 | 140513452 | 140552250 | 0.005968871 | 28.82275132 | Hyper-methylated | PCa\|NT DMRs |
| chr3 | 140949414 | 140950323 | 2.99E−05 | 21.14234234 | Hyper-methylated | PCa\|NT DMRs |
| chr3 | 141087119 | 141087314 | 6.38E−06 | −39.4 | Hypo-methylated | PCa\|NT DMRs |
| chr3 | 141380054 | 141380400 | 0.001969641 | 28.36031746 | Hyper-methylated | PCa\|NT DMRs |
| chr3 | 141989206 | 141989938 | 0.004095673 | 32.18253968 | Hyper-methylated | PCa\|NT DMRs |
| chr3 | 142443823 | 142443934 | 6.42E−05 | 21.32010582 | Hyper-methylated | PCa\|NT DMRs |
| chr3 | 142895178 | 142895211 | 0.005968871 | −51.82857143 | Hypo-methylated | PCa\|NT DMRs |
| chr3 | 144509945 | 144818559 | 6.68E−10 | −32.58180272 | Hypo-methylated | PCa\|NT DMRs |
| chr3 | 145249570 | 145378745 | 0.005513661 | −30.57687075 | Hypo-methylated | PCa\|NT DMRs |
| chr3 | 147290242 | 147580795 | 1.23E−07 | −37.58550725 | Hypo-methylated | PCa\|NT DMRs |
| chr3 | 147706513 | 147776676 | 7.55E−05 | −27.39598997 | Hypo-methylated | PCa\|NT DMRs |
| chr3 | 147998621 | 147998677 | 0.000205174 | −29.99345238 | Hypo-methylated | PCa\|NT DMRs |
| chr3 | 149261225 | 149265370 | 0.00023371 | −30.05714286 | Hypo-methylated | PCa\|NT DMRs |
| chr3 | 152821757 | 152821793 | 0.005968871 | 34.58 | Hyper-methylated | PCa\|NT DMRs |
| chr3 | 156009117 | 156009490 | 9.92E−08 | 24.39160998 | Hyper-methylated | PCa\|NT DMRs |
| chr3 | 156533849 | 156534411 | 1.67E−10 | 29.01267281 | Hyper-methylated | PCa\|NT DMRs |
| chr3 | 156827612 | 156843435 | 0.000149331 | −34.85357143 | Hypo-methylated | PCa\|NT DMRs |
| chr3 | 159725550 | 159725722 | 0.00064571 | −35.44897959 | Hypo-methylated | PCa\|NT DMRs |
| chr3 | 159942271 | 159943073 | 8.98E−05 | 21.94404762 | Hyper-methylated | PCa\|NT DMRs |
| chr3 | 163285117 | 163643541 | 1.67E−10 | −29.62596006 | Hypo-methylated | PCa\|NT DMRs |
| chr3 | 163726905 | 164029516 | 9.82E−07 | −28.14616977 | Hypo-methylated | PCa\|NT DMRs |
| chr3 | 164436187 | 164537373 | 3.94E−09 | −24.6079932 | Hypo-methylated | PCa\|NT DMRs |
| chr3 | 164583687 | 164588390 | 1.67E−08 | −32.02539683 | Hypo-methylated | PCa\|NT DMRs |
| chr3 | 164912468 | 164912554 | 0.002097193 | 25.46122449 | Hyper-methylated | PCa\|NT DMRs |
| chr3 | 164923574 | 164989437 | 0.005968871 | −28.83809524 | Hypo-methylated | PCa\|NT DMRs |
| chr3 | 166984456 | 166984739 | 0.000205174 | −25.83928571 | Hypo-methylated | PCa\|NT DMRs |
| chr3 | 167126693 | 167377217 | 0.001969641 | −29.18650794 | Hypo-methylated | PCa\|NT DMRs |
| chr3 | 168005442 | 168834269 | 2.31E−13 | −28.96774892 | Hypo-methylated | PCa\|NT DMRs |
| chr3 | 169129528 | 169362341 | 6.77E−09 | −28.18752053 | Hypo-methylated | PCa\|NT DMRs |
| chr3 | 169376578 | 169379504 | 6.78E−07 | 26.66363636 | Hyper-methylated | PCa\|NT DMRs |
| chr3 | 169719911 | 169720363 | 0.001291665 | 33.6991342 | Hyper-methylated | PCa\|NT DMRs |
| chr3 | 170745826 | 170746208 | 0.000277103 | 40.18315018 | Hyper-methylated | PCa\|NT DMRs |
| chr3 | 171175816 | 171176142 | 2.86E−05 | 45.59340659 | Hyper-methylated | PCa\|NT DMRs |
| chr3 | 171846800 | 171854790 | 0.006006923 | 28.91680672 | Hyper-methylated | PCa\|NT DMRs |
| chr3 | 172618473 | 172881213 | 7.58E−07 | −24.47204969 | Hypo-methylated | PCa\|NT DMRs |
| chr3 | 172993980 | 173030958 | 0.003290817 | −24.49642857 | Hypo-methylated | PCa\|NT DMRs |
| chr3 | 177310875 | 177311090 | 0.003871035 | −26.68154762 | Hypo-methylated | PCa\|NT DMRs |
| chr3 | 179182653 | 179182799 | 0.005968871 | 45.27619048 | Hyper-methylated | PCa\|NT DMRs |
| chr3 | 179280026 | 179280075 | 0.001969641 | −43.23809524 | Hypo-methylated | PCa\|NT DMRs |
| chr3 | 180588007 | 180588082 | 0.000205174 | 26.35714286 | Hyper-methylated | PCa\|NT DMRs |
| chr3 | 181413070 | 181414195 | 3.74E−12 | 26.01486643 | Hyper-methylated | PCa\|NT DMRs |
| chr3 | 183258704 | 183272723 | 2.52E−05 | 35.58403361 | Hyper-methylated | PCa\|NT DMRs |
| chr3 | 183542549 | 183542851 | 8.68E−12 | 23.00225564 | Hyper-methylated | PCa\|NT DMRs |
| chr3 | 183913838 | 183925396 | 0.000696555 | −20.03896104 | Hypo-methylated | PCa\|NT DMRs |
| chr3 | 184277166 | 184277439 | 0.005968871 | −45.54285714 | Hypo-methylated | PCa\|NT DMRs |
| chr3 | 184280190 | 184280945 | 3.08E−06 | −29.04702381 | Hypo-methylated | PCa\|NT DMRs |
| chr3 | 184404896 | 184405563 | 0.007679542 | 34.01111111 | Hyper-methylated | PCa\|NT DMRs |
| chr3 | 184893503 | 184893781 | 0.005513661 | 37.23469388 | Hyper-methylated | PCa\|NT DMRs |
| chr3 | 186490372 | 186490791 | 2.41E−08 | 43.01573499 | Hyper-methylated | PCa\|NT DMRs |
| chr3 | 186542783 | 186543274 | 0.000575877 | 29.24632035 | Hyper-methylated | PCa\|NT DMRs |
| chr3 | 186822222 | 186844110 | 0.001789867 | −41.83280423 | Hypo-methylated | PCa\|NT DMRs |
| chr3 | 187676564 | 187683317 | 0.004629421 | 49.9952381 | Hyper-methylated | PCa\|NT DMRs |
| chr3 | 188012863 | 188012956 | 0.005968871 | −50.23333333 | Hypo-methylated | PCa\|NT DMRs |
| chr3 | 191167788 | 191661655 | 8.95E−16 | −33.16402116 | Hypo-methylated | PCa\|NT DMRs |
| chr3 | 192530105 | 192623547 | 9.33E−10 | −28.51970443 | Hypo-methylated | PCa\|NT DMRs |
| chr3 | 192670735 | 192751762 | 0.00015808 | −30.85612245 | Hypo-methylated | PCa\|NT DMRs |
| chr3 | 192760612 | 192943009 | 4.14E−08 | −35.67043651 | Hypo-methylated | PCa\|NT DMRs |
| chr3 | 192974771 | 193218451 | 8.70E−08 | −32.08710317 | Hypo-methylated | PCa\|NT DMRs |
| chr3 | 193576680 | 193599889 | 1.42E−07 | 28.88779762 | Hyper-methylated | PCa\|NT DMRs |
| chr3 | 193661153 | 193661325 | 0.001969641 | −30.45634921 | Hypo-methylated | PCa\|NT DMRs |
| chr3 | 193755127 | 193788568 | 2.58E−07 | 31.84500768 | Hyper-methylated | PCa\|NT DMRs |
| chr3 | 193983048 | 193983157 | 0.001969641 | −21.97959184 | Hypo-methylated | PCa\|NT DMRs |
| chr3 | 194018000 | 194018209 | 1.97E−05 | 47.22619048 | Hyper-methylated | PCa\|NT DMRs |
| chr3 | 194034015 | 194034113 | 0.00068166 | 23.18452381 | Hyper-methylated | PCa\|NT DMRs |
| chr3 | 194116989 | 194118924 | 3.32E−14 | 40.01227513 | Hyper-methylated | PCa\|NT DMRs |
| chr3 | 194845951 | 194865631 | 0.009050681 | 20.45714286 | Hyper-methylated | PCa\|NT DMRs |
| chr3 | 194899525 | 194907898 | 0.0029761 | 27.63406593 | Hyper-methylated | PCa\|NT DMRs |
| chr3 | 195488494 | 195490917 | 6.49E−06 | 20.99220779 | Hyper-methylated | PCa\|NT DMRs |
| chr3 | 195566483 | 195570580 | 0.000777687 | −22.31428571 | Hypo-methylated | PCa\|NT DMRs |
| chr3 | 195583593 | 195586665 | 0.002585277 | 46.58630952 | Hyper-methylated | PCa\|NT DMRs |
| chr3 | 195682379 | 195685848 | 0.000398029 | 35.91208791 | Hyper-methylated | PCa\|NT DMRs |
| chr3 | 195749097 | 195753109 | 0.002193447 | −33.395671 | Hypo-methylated | PCa\|NT DMRs |
| chr3 | 195920950 | 195921816 | 0.007338757 | 30.24404762 | Hyper-methylated | PCa\|NT DMRs |
| chr3 | 195929165 | 195929663 | 0.000339627 | 29.575 | Hyper-methylated | PCa\|NT DMRs |
| chr3 | 195942925 | 195943487 | 2.65E−07 | −40.02142857 | Hypo-methylated | PCa\|NT DMRs |
| chr3 | 195947033 | 195948885 | 0.007991909 | 24.00805861 | Hyper-methylated | PCa\|NT DMRs |
| chr3 | 195950175 | 195950324 | 0.001969641 | −50 | Hypo-methylated | PCa\|NT DMRs |
| chr3 | 196187786 | 196187834 | 0.005968871 | −36.22857143 | Hypo-methylated | PCa\|NT DMRs |
| chr3 | 196367507 | 196367833 | 6.93E−06 | 49.64761905 | Hyper-methylated | PCa\|NT DMRs |
| chr3 | 196387377 | 196393760 | 9.49E−05 | −39.3967033 | Hypo-methylated | PCa\|NT DMRs |
| chr3 | 196397715 | 196399708 | 0.007628253 | −21.14965986 | Hypo-methylated | PCa\|NT DMRs |
| chr3 | 196716852 | 196717004 | 0.001181579 | −40.49387755 | Hypo-methylated | PCa\|NT DMRs |
| chr3 | 196738019 | 196738112 | 0.005968871 | −43.44761905 | Hypo-methylated | PCa\|NT DMRs |
| chr3 | 197081608 | 197081754 | 0.000205174 | −34.03571429 | Hypo-methylated | PCa\|NT DMRs |
| chr3 | 197260360 | 197260527 | 0.001969641 | 41.52040816 | Hyper-methylated | PCa\|NT DMRs |
| chr3 | 197263525 | 197267031 | 0.001775064 | −45.94047619 | Hypo-methylated | PCa\|NT DMRs |
| chr3 | 197269671 | 197269871 | 0.00064571 | 39.08503401 | Hyper-methylated | PCa\|NT DMRs |
| chr3 | 197326607 | 197327213 | 0.000601868 | −20.30782313 | Hypo-methylated | PCa\|NT DMRs |
| chr3 | 197364016 | 197365314 | 0.005257942 | 23.72825397 | Hyper-methylated | PCa\|NT DMRs |
| chr3 | 197678267 | 197684523 | 0.001480644 | −37.46095238 | Hypo-methylated | PCa\|NT DMRs |
| chr4 | 382667 | 383850 | 0.001969641 | −33.11746032 | Hypo-methylated | PCa\|NT DMRs |
| chr4 | 583022 | 585254 | 0.008482405 | −24.05714286 | Hypo-methylated | PCa\|NT DMRs |
| chr4 | 601111 | 606632 | 9.23E−05 | −25.72521008 | Hypo-methylated | PCa\|NT DMRs |
| chr4 | 658691 | 666698 | 5.65E−13 | −23.05869565 | Hypo-methylated | PCa\|NT DMRs |
| chr4 | 684991 | 686987 | 0.000325529 | −32.9047619 | Hypo-methylated | PCa\|NT DMRs |
| chr4 | 720211 | 720653 | 0.005513661 | 31.87278912 | Hyper-methylated | PCa\|NT DMRs |
| chr4 | 778703 | 781744 | 5.70E−22 | 34.84824659 | Hyper-methylated | PCa\|NT DMRs |
| chr4 | 827973 | 828360 | 3.52E−06 | −20.64393241 | Hypo-methylated | PCa\|NT DMRs |
| chr4 | 942132 | 942382 | 0.001480644 | −22.68571429 | Hypo-methylated | PCa\|NT DMRs |
| chr4 | 961400 | 962298 | 7.98E−08 | 23.56616008 | Hyper-methylated | PCa\|NT DMRs |
| chr4 | 972502 | 973371 | 0.000179749 | 39.76817043 | Hyper-methylated | PCa\|NT DMRs |
| chr4 | 997048 | 997871 | 3.53E−08 | 23.85668203 | Hyper-methylated | PCa\|NT DMRs |
| chr4 | 1029311 | 1029676 | 1.23E−10 | −32.81228879 | Hypo-methylated | PCa\|NT DMRs |
| chr4 | 1032712 | 1035418 | 1.27E−05 | −41.11038961 | Hypo-methylated | PCa\|NT DMRs |
| chr4 | 1044864 | 1049270 | 1.10E−07 | −39.62981366 | Hypo-methylated | PCa\|NT DMRs |
| chr4 | 1049394 | 1049923 | 1.67E−07 | 46.01360544 | Hyper-methylated | PCa\|NT DMRs |
| chr4 | 1050670 | 1053023 | 3.13E−06 | −35.13854167 | Hypo-methylated | PCa\|NT DMRs |
| chr4 | 1219786 | 1220184 | 6.84E−06 | −20.43697479 | Hypo-methylated | PCa\|NT DMRs |
| chr4 | 1221838 | 1222191 | 0.001015461 | 25.77380952 | Hyper-methylated | PCa\|NT DMRs |
| chr4 | 1288917 | 1289089 | 0.000377317 | 28.31785714 | Hyper-methylated | PCa\|NT DMRs |
| chr4 | 1398732 | 1399206 | 6.67E−07 | 20.03513957 | Hyper-methylated | PCa\|NT DMRs |
| chr4 | 1399634 | 1399999 | 6.17E−07 | 20.23904762 | Hyper-methylated | PCa\|NT DMRs |
| chr4 | 1411454 | 1413594 | 0.005685503 | −22.30793651 | Hypo-methylated | PCa\|NT DMRs |
| chr4 | 1419622 | 1419973 | 0.001083007 | −26.79285714 | Hypo-methylated | PCa\|NT DMRs |
| chr4 | 1490858 | 1491097 | 1.16E−05 | −27.80498866 | Hypo-methylated | PCa\|NT DMRs |
| chr4 | 1555825 | 1557487 | 1.07E−05 | −26.10535714 | Hypo-methylated | PCa\|NT DMRs |
| chr4 | 1642065 | 1642543 | 0.000199784 | −31.33406593 | Hypo-methylated | PCa\|NT DMRs |
| chr4 | 1720110 | 1720849 | 0.00017871 | −38.9547619 | Hypo-methylated | PCa\|NT DMRs |
| chr4 | 1724680 | 1726858 | 0.007347803 | 29.72619048 | Hyper-methylated | PCa\|NT DMRs |
| chr4 | 1748760 | 1749266 | 7.74E−06 | −27.23388278 | Hypo-methylated | PCa\|NT DMRs |
| chr4 | 1753484 | 1755408 | 0.001805038 | 27.78497024 | Hyper-methylated | PCa\|NT DMRs |
| chr4 | 1761441 | 1761940 | 0.006225823 | 29.85714286 | Hyper-methylated | PCa\|NT DMRs |
| chr4 | 1762383 | 1763101 | 0.002217636 | −24.14711779 | Hypo-methylated | PCa\|NT DMRs |
| chr4 | 2059259 | 2060359 | 0.005925694 | 24.14384921 | Hyper-methylated | PCa\|NT DMRs |
| chr4 | 2066003 | 2068267 | 1.96E−08 | −30.76893939 | Hypo-methylated | PCa\|NT DMRs |
| chr4 | 2460407 | 2460490 | 0.001969641 | −42.95238095 | Hypo-methylated | PCa\|NT DMRs |
| chr4 | 2813250 | 2814969 | 0.00309961 | 32.30896359 | Hyper-methylated | PCa\|NT DMRs |
| chr4 | 3042816 | 3043909 | 2.82E−10 | 28.96311858 | Hyper-methylated | PCa\|NT DMRs |
| chr4 | 3081508 | 3082118 | 0.005968871 | 36.65238095 | Hyper-methylated | PCa\|NT DMRs |
| chr4 | 3322523 | 3322749 | 1.97E−05 | −26.15952381 | Hypo-methylated | PCa\|NT DMRs |
| chr4 | 3374739 | 3375210 | 0.00187042 | 35.19047619 | Hyper-methylated | PCa\|NT DMRs |
| chr4 | 3461141 | 3463747 | 0.00068166 | −41.06785714 | Hypo-methylated | PCa\|NT DMRs |
| chr4 | 3472262 | 3472432 | 6.42E−05 | −48.11904762 | Hypo-methylated | PCa\|NT DMRs |
| chr4 | 3485298 | 3485482 | 1.02E−06 | 24.41478697 | Hyper-methylated | PCa\|NT DMRs |
| chr4 | 3485917 | 3486308 | 3.24E−10 | −40.02873563 | Hypo-methylated | PCa\|NT DMRs |
| chr4 | 3516421 | 3516501 | 0.002097193 | 33.08979592 | Hyper-methylated | PCa\|NT DMRs |
| chr4 | 3572867 | 3574998 | 0.009214933 | −22.77310924 | Hypo-methylated | PCa\|NT DMRs |
| chr4 | 3615818 | 3616211 | 8.17E−05 | −36.22294372 | Hypo-methylated | PCa\|NT DMRs |
| chr4 | 3703042 | 3703291 | 0.005438982 | −24.41761905 | Hypo-methylated | PCa\|NT DMRs |
| chr4 | 3731958 | 3732297 | 0.001969641 | −37.72222222 | Hypo-methylated | PCa\|NT DMRs |
| chr4 | 3773821 | 3774409 | 0.008581782 | −21.32142857 | Hypo-methylated | PCa\|NT DMRs |
| chr4 | 3829455 | 3829864 | 1.97E−05 | −40.09285714 | Hypo-methylated | PCa\|NT DMRs |
| chr4 | 3866527 | 3866708 | 0.003622801 | −26.42460317 | Hypo-methylated | PCa\|NT DMRs |
| chr4 | 3873093 | 3873135 | 0.00023371 | 31.27142857 | Hyper-methylated | PCa\|NT DMRs |
| chr4 | 3881315 | 3881658 | 1.67E−05 | 21.23809524 | Hyper-methylated | PCa\|NT DMRs |
| chr4 | 3947643 | 3947729 | 0.000205174 | −55.10714286 | Hypo-methylated | PCa\|NT DMRs |
| chr4 | 4149554 | 4162367 | 0.003871035 | −32.16011905 | Hypo-methylated | PCa\|NT DMRs |
| chr4 | 4167004 | 4168811 | 0.002111644 | −30.37875458 | Hypo-methylated | PCa\|NT DMRs |
| chr4 | 4177672 | 4202128 | 2.38E−05 | −23.69738095 | Hypo-methylated | PCa\|NT DMRs |
| chr4 | 4337976 | 4341205 | 1.97E−05 | −32.82666667 | Hypo-methylated | PCa\|NT DMRs |
| chr4 | 4508991 | 4509381 | 6.42E−05 | −63.63492063 | Hypo-methylated | PCa\|NT DMRs |
| chr4 | 4732003 | 4732140 | 0.005968871 | −23.0047619 | Hypo-methylated | PCa\|NT DMRs |
| chr4 | 4765203 | 4765535 | 1.76E−05 | 34.97767857 | Hyper-methylated | PCa\|NT DMRs |
| chr4 | 4859674 | 4859847 | 5.84E−05 | 43.86111111 | Hyper-methylated | PCa\|NT DMRs |
| chr4 | 5841992 | 5842256 | 0.000967268 | −27.31746032 | Hypo-methylated | PCa\|NT DMRs |
| chr4 | 5867725 | 5868199 | 0.001789867 | 21.12962963 | Hyper-methylated | PCa\|NT DMRs |
| chr4 | 5872535 | 5872659 | 0.005513661 | 28.11309524 | Hyper-methylated | PCa\|NT DMRs |
| chr4 | 5873074 | 5879341 | 0.002097193 | −27.46938776 | Hypo-methylated | PCa\|NT DMRs |
| chr4 | 5892244 | 5892706 | 0.000487056 | 21.31792717 | Hyper-methylated | PCa\|NT DMRs |
| chr4 | 5899432 | 5907689 | 0.00449535 | −21.55438596 | Hypo-methylated | PCa\|NT DMRs |
| chr4 | 5930354 | 5957181 | 1.28E−08 | −26.77085714 | Hypo-methylated | PCa\|NT DMRs |
| chr4 | 5990228 | 5997337 | 4.45E−08 | −27.90634921 | Hypo-methylated | PCa\|NT DMRs |
| chr4 | 6025011 | 6026325 | 0.000117699 | −22.13246753 | Hypo-methylated | PCa\|NT DMRs |
| chr4 | 6310716 | 6314451 | 6.71E−12 | 27.86571429 | Hyper-methylated | PCa\|NT DMRs |
| chr4 | 6381815 | 6382787 | 0.001181579 | 36.55102041 | Hyper-methylated | PCa\|NT DMRs |
| chr4 | 6390691 | 6392474 | 2.41E−05 | 20.48271605 | Hyper-methylated | PCa\|NT DMRs |
| chr4 | 6578407 | 6578819 | 0.005513661 | −32.48809524 | Hypo-methylated | PCa\|NT DMRs |
| chr4 | 6659273 | 6660594 | 6.20E−09 | 35.41931217 | Hyper-methylated | PCa\|NT DMRs |
| chr4 | 6679681 | 6679830 | 0.005968871 | 35.7047619 | Hyper-methylated | PCa\|NT DMRs |
| chr4 | 6694506 | 6696428 | 5.42E−05 | 40.65277778 | Hyper-methylated | PCa\|NT DMRs |
| chr4 | 6883830 | 6883872 | 0.004095673 | −21.6 | Hypo-methylated | PCa\|NT DMRs |
| chr4 | 6891128 | 6894158 | 5.84E−05 | 23.74141414 | Hyper-methylated | PCa\|NT DMRs |
| chr4 | 6939615 | 6940777 | 0.000589422 | 25.93859649 | Hyper-methylated | PCa\|NT DMRs |
| chr4 | 7151787 | 7152611 | 9.62E−06 | −35.4537037 | Hypo-methylated | PCa\|NT DMRs |
| chr4 | 7185889 | 7193971 | 0.008234951 | −25.27285714 | Hypo-methylated | PCa\|NT DMRs |
| chr4 | 7297084 | 7298954 | 0.000167577 | −37.04613095 | Hypo-methylated | PCa\|NT DMRs |
| chr4 | 7306667 | 7308011 | 7.95E−05 | −25.05860806 | Hypo-methylated | PCa\|NT DMRs |
| chr4 | 7328211 | 7334272 | 5.34E−10 | −23.71273533 | Hypo-methylated | PCa\|NT DMRs |
| chr4 | 7336839 | 7337587 | 0.002879175 | −23.08683473 | Hypo-methylated | PCa\|NT DMRs |
| chr4 | 7363202 | 7370060 | 4.14E−05 | −34.61349206 | Hypo-methylated | PCa\|NT DMRs |
| chr4 | 7394782 | 7401877 | 1.22E−05 | −26.90785714 | Hypo-methylated | PCa\|NT DMRs |
| chr4 | 7462714 | 7463938 | 0.003391914 | −29.11851852 | Hypo-methylated | PCa\|NT DMRs |
| chr4 | 7512128 | 7512451 | 0.000197903 | −22.3952381 | Hypo-methylated | PCa\|NT DMRs |
| chr4 | 7520455 | 7520899 | 2.01E−07 | −48.115 | Hypo-methylated | PCa\|NT DMRs |
| chr4 | 7560377 | 7566963 | 9.22E−08 | −30.11704261 | Hypo-methylated | PCa\|NT DMRs |
| chr4 | 7595386 | 7604001 | 0.001391421 | −21.03582766 | Hypo-methylated | PCa\|NT DMRs |
| chr4 | 7632330 | 7634423 | 8.36E−06 | −41.17989418 | Hypo-methylated | PCa\|NT DMRs |
| chr4 | 7693291 | 7699047 | 3.13E−07 | −31.19603175 | Hypo-methylated | PCa\|NT DMRs |
| chr4 | 7829769 | 7829989 | 0.00064571 | 38.63673469 | Hyper-methylated | PCa\|NT DMRs |
| chr4 | 7958232 | 7958475 | 0.005968871 | 21.24095238 | Hyper-methylated | PCa\|NT DMRs |
| chr4 | 8184991 | 8185183 | 0.001969641 | −65.86666667 | Hypo-methylated | PCa\|NT DMRs |
| chr4 | 8191543 | 8193601 | 2.57E−05 | 35.57352941 | Hyper-methylated | PCa\|NT DMRs |
| chr4 | 8201515 | 8204391 | 6.48E−12 | 41.05639589 | Hyper-methylated | PCa\|NT DMRs |
| chr4 | 8216793 | 8216969 | 0.000974795 | −55.5 | Hypo-methylated | PCa\|NT DMRs |
| chr4 | 8228509 | 8229648 | 8.39E−05 | 41.0156746 | Hyper-methylated | PCa\|NT DMRs |
| chr4 | 8261808 | 8262851 | 0.002562638 | −26.54761905 | Hypo-methylated | PCa\|NT DMRs |
| chr4 | 8263587 | 8266746 | 8.96E−05 | 42.93174603 | Hyper-methylated | PCa\|NT DMRs |
| chr4 | 8271056 | 8271692 | 8.43E−07 | 25.18376068 | Hyper-methylated | PCa\|NT DMRs |
| chr4 | 8274761 | 8276039 | 0.005835969 | 26.23396825 | Hyper-methylated | PCa\|NT DMRs |
| chr4 | 8429102 | 8429459 | 0.008345196 | −43.4875 | Hypo-methylated | PCa\|NT DMRs |
| chr4 | 8453854 | 8468108 | 2.79E−05 | −20.88919414 | Hypo-methylated | PCa\|NT DMRs |
| chr4 | 9539104 | 9679680 | 1.27E−10 | −36.01693122 | Hypo-methylated | PCa\|NT DMRs |
| chr4 | 9693765 | 9701202 | 0.00179283 | −30.25297619 | Hypo-methylated | PCa\|NT DMRs |
| chr4 | 9705424 | 9705454 | 0.005968871 | −22.53333333 | Hypo-methylated | PCa\|NT DMRs |
| chr4 | 9733545 | 9780270 | 1.36E−08 | −28.18826945 | Hypo-methylated | PCa\|NT DMRs |
| chr4 | 9784902 | 9916437 | 4.90E−08 | −32.93290043 | Hypo-methylated | PCa\|NT DMRs |
| chr4 | 10035430 | 10035609 | 0.005968871 | −35.48571429 | Hypo-methylated | PCa\|NT DMRs |
| chr4 | 10116810 | 10117450 | 0.000556158 | 26.3847619 | Hyper-methylated | PCa\|NT DMRs |
| chr4 | 10256054 | 10385440 | 6.69E−05 | −29.14619048 | Hypo-methylated | PCa\|NT DMRs |
| chr4 | 11370382 | 11370691 | 1.67E−08 | 30.99487179 | Hyper-methylated | PCa\|NT DMRs |
| chr4 | 11558925 | 11652784 | 0.003007728 | −30.43150183 | Hypo-methylated | PCa\|NT DMRs |
| chr4 | 12538726 | 12688866 | 3.05E−06 | −32.96263736 | Hypo-methylated | PCa\|NT DMRs |
| chr4 | 12765196 | 12785349 | 0.000117699 | −24.64502165 | Hypo-methylated | PCa\|NT DMRs |
| chr4 | 13691364 | 14512872 | 1.10E−19 | −29.89767316 | Hypo-methylated | PCa\|NT DMRs |
| chr4 | 16229075 | 16240731 | 0.006195268 | 35.44480519 | Hyper-methylated | PCa\|NT DMRs |
| chr4 | 16322938 | 16386559 | 6.42E−05 | −30.68148148 | Hypo-methylated | PCa\|NT DMRs |
| chr4 | 17439111 | 17463098 | 0.001969641 | −29.5952381 | Hypo-methylated | PCa\|NT DMRs |
| chr4 | 17783241 | 17783692 | 1.02E−05 | 25.68419048 | Hyper-methylated | PCa\|NT DMRs |
| chr4 | 20256532 | 20256793 | 6.90E−08 | 29.06207483 | Hyper-methylated | PCa\|NT DMRs |
| chr4 | 24374606 | 24462075 | 5.78E−09 | −31.72704762 | Hypo-methylated | PCa\|NT DMRs |
| chr4 | 24800957 | 24801882 | 5.15E−15 | 37.06678424 | Hyper-methylated | PCa\|NT DMRs |
| chr4 | 24918971 | 24919056 | 0.002826041 | 30.17006803 | Hyper-methylated | PCa\|NT DMRs |
| chr4 | 25315220 | 25315306 | 6.42E−05 | −20.0952381 | Hypo-methylated | PCa\|NT DMRs |
| chr4 | 25845316 | 25862708 | 0.001428615 | 23.99107143 | Hyper-methylated | PCa\|NT DMRs |
| chr4 | 25880852 | 25894696 | 0.00064571 | −28.13945578 | Hypo-methylated | PCa\|NT DMRs |
| chr4 | 26222965 | 26228960 | 0.003587573 | −24.95454545 | Hypo-methylated | PCa\|NT DMRs |
| chr4 | 26275399 | 26277170 | 2.05E−05 | 39.38831169 | Hyper-methylated | PCa\|NT DMRs |
| chr4 | 26492350 | 26492489 | 0.008482405 | −30.2 | Hypo-methylated | PCa\|NT DMRs |
| chr4 | 27517943 | 27815111 | 8.47E−09 | −25.84334975 | Hypo-methylated | PCa\|NT DMRs |
| chr4 | 28977366 | 29260233 | 1.60E−11 | −26.73258145 | Hypo-methylated | PCa\|NT DMRs |
| chr4 | 29370807 | 29544767 | 1.92E−05 | −27.37170868 | Hypo-methylated | PCa\|NT DMRs |
| chr4 | 29705132 | 29816256 | 0.000117738 | −30.36825397 | Hypo-methylated | PCa\|NT DMRs |
| chr4 | 32417159 | 32681598 | 1.23E−14 | −31.82037471 | Hypo-methylated | PCa\|NT DMRs |
| chr4 | 32765622 | 33252003 | 4.35E−10 | −31.26654457 | Hypo-methylated | PCa\|NT DMRs |
| chr4 | 35081335 | 35132653 | 9.98E−10 | −30.38857143 | Hypo-methylated | PCa\|NT DMRs |
| chr4 | 37409388 | 37430650 | 0.001969641 | −22.70714286 | Hypo-methylated | PCa\|NT DMRs |
| chr4 | 37456149 | 37536784 | 6.11E−05 | −25.58536155 | Hypo-methylated | PCa\|NT DMRs |
| chr4 | 38138915 | 38139074 | 0.00064571 | 21.77006803 | Hyper-methylated | PCa\|NT DMRs |
| chr4 | 39483130 | 39483195 | 0.001969641 | 38.71768707 | Hyper-methylated | PCa\|NT DMRs |
| chr4 | 40516823 | 40518046 | 0.000392715 | −35.68619048 | Hypo-methylated | PCa\|NT DMRs |
| chr4 | 40564371 | 40564644 | 0.002437742 | 31.29153439 | Hyper-methylated | PCa\|NT DMRs |
| chr4 | 41258790 | 41259098 | 5.11E−13 | 29.49385965 | Hyper-methylated | PCa\|NT DMRs |
| chr4 | 41634825 | 41634912 | 0.005968871 | 43.08571429 | Hyper-methylated | PCa\|NT DMRs |
| chr4 | 41785626 | 41790072 | 0.000226106 | −22.67765568 | Hypo-methylated | PCa\|NT DMRs |
| chr4 | 43062921 | 43171463 | 0.000118733 | −29.75661376 | Hypo-methylated | PCa\|NT DMRs |
| chr4 | 46481587 | 46701221 | 1.42E−05 | −36.74659864 | Hypo-methylated | PCa\|NT DMRs |
| chr4 | 46726479 | 46726517 | 0.000260936 | 24.14285714 | Hyper-methylated | PCa\|NT DMRs |
| chr4 | 46768438 | 46833223 | 1.72E−11 | −23.9175 | Hypo-methylated | PCa\|NT DMRs |
| chr4 | 46853075 | 46956338 | 0.00064571 | −38.8 | Hypo-methylated | PCa\|NT DMRs |
| chr4 | 47222577 | 47387862 | 1.75E−06 | −30.41322751 | Hypo-methylated | PCa\|NT DMRs |
| chr4 | 48946604 | 48987966 | 0.002400984 | 26.83174603 | Hyper-methylated | PCa\|NT DMRs |
| chr4 | 53060099 | 53156841 | 6.42E−05 | −39.47883598 | Hypo-methylated | PCa\|NT DMRs |
| chr4 | 53708455 | 53714838 | 0.00064571 | −43.42176871 | Hypo-methylated | PCa\|NT DMRs |
| chr4 | 54967655 | 54976202 | 4.10E−28 | 36.93661376 | Hyper-methylated | PCa\|NT DMRs |
| chr4 | 55015540 | 55035219 | 0.000744739 | 26.81180952 | Hyper-methylated | PCa\|NT DMRs |
| chr4 | 55092628 | 55093783 | 6.30E−14 | 24.46436012 | Hyper-methylated | PCa\|NT DMRs |
| chr4 | 56023416 | 56023965 | 0.004505524 | 24.05882353 | Hyper-methylated | PCa\|NT DMRs |
| chr4 | 56552514 | 56574020 | 6.42E−05 | −32.2989418 | Hypo-methylated | PCa\|NT DMRs |
| chr4 | 56799018 | 56799469 | 0.001969641 | −26.3984127 | Hypo-methylated | PCa\|NT DMRs |
| chr4 | 56964950 | 56965146 | 0.00064571 | −29.01632653 | Hypo-methylated | PCa\|NT DMRs |
| chr4 | 57594501 | 57595240 | 0.005968871 | −38.73809524 | Hypo-methylated | PCa\|NT DMRs |
| chr4 | 57603960 | 57608792 | 0.001969641 | −54.53333333 | Hypo-methylated | PCa\|NT DMRs |
| chr4 | 58166581 | 58188744 | 6.42E−05 | −37.15502646 | Hypo-methylated | PCa\|NT DMRs |
| chr4 | 58596163 | 59038524 | 1.39E−12 | −31.19580499 | Hypo-methylated | PCa\|NT DMRs |
| chr4 | 59227061 | 60313531 | 1.05E−29 | −29.63496473 | Hypo-methylated | PCa\|NT DMRs |
| chr4 | 60405931 | 60834746 | 5.90E−07 | −28.43638095 | Hypo-methylated | PCa\|NT DMRs |
| chr4 | 62224037 | 62253365 | 0.001969641 | −30.22777778 | Hypo-methylated | PCa\|NT DMRs |
| chr4 | 62527830 | 62641591 | 5.87E−05 | −30.25863095 | Hypo-methylated | PCa\|NT DMRs |
| chr4 | 63203786 | 63675452 | 1.46E−14 | −34.23105159 | Hypo-methylated | PCa\|NT DMRs |
| chr4 | 63721604 | 64214752 | 1.47E−15 | −34.50452771 | Hypo-methylated | PCa\|NT DMRs |
| chr4 | 64294514 | 64899391 | 2.90E−13 | −28.92775041 | Hypo-methylated | PCa\|NT DMRs |
| chr4 | 64903156 | 64904679 | 1.42E−05 | −28.18333333 | Hypo-methylated | PCa\|NT DMRs |
| chr4 | 66027772 | 66060053 | 0.003871035 | −21.87083333 | Hypo-methylated | PCa\|NT DMRs |
| chr4 | 67076764 | 67237811 | 0.000117507 | −32.46753247 | Hypo-methylated | PCa\|NT DMRs |
| chr4 | 67575557 | 67786456 | 1.49E−09 | −29.11773399 | Hypo-methylated | PCa\|NT DMRs |
| chr4 | 68141285 | 68152698 | 1.69E−05 | −32.23428571 | Hypo-methylated | PCa\|NT DMRs |
| chr4 | 71517294 | 71517351 | 0.005968871 | −39.82857143 | Hypo-methylated | PCa\|NT DMRs |
| chr4 | 71703513 | 71703671 | 0.005968871 | −36.62857143 | Hypo-methylated | PCa\|NT DMRs |
| chr4 | 74470061 | 74470116 | 0.001181579 | −39.5170068 | Hypo-methylated | PCa\|NT DMRs |
| chr4 | 74735344 | 74735544 | 0.000826653 | 24.62202381 | Hyper-methylated | PCa\|NT DMRs |
| chr4 | 79971150 | 79971392 | 6.42E−05 | −66.02116402 | Hypo-methylated | PCa\|NT DMRs |
| chr4 | 81128344 | 81128545 | 0.001391421 | 20.66233766 | Hyper-methylated | PCa\|NT DMRs |
| chr4 | 81188030 | 81189752 | 2.66E−09 | 25.02551893 | Hyper-methylated | PCa\|NT DMRs |
| chr4 | 81222832 | 81229128 | 0.002097193 | −42.65306122 | Hypo-methylated | PCa\|NT DMRs |
| chr4 | 81257352 | 81257875 | 0.007560755 | 21.65079365 | Hyper-methylated | PCa\|NT DMRs |
| chr4 | 84029495 | 84030975 | 0.006207271 | 44.14285714 | Hyper-methylated | PCa\|NT DMRs |
| chr4 | 85246157 | 85246262 | 0.001344447 | 35.33928571 | Hyper-methylated | PCa\|NT DMRs |
| chr4 | 86049074 | 86276626 | 0.002529619 | −33.2996337 | Hypo-methylated | PCa\|NT DMRs |
| chr4 | 87770576 | 87793106 | 0.00641359 | −34.06984127 | Hypo-methylated | PCa\|NT DMRs |
| chr4 | 87814459 | 87814664 | 0.007959104 | −28.55102041 | Hypo-methylated | PCa\|NT DMRs |
| chr4 | 90600720 | 90600871 | 0.005968871 | 40.15238095 | Hyper-methylated | PCa\|NT DMRs |
| chr4 | 93397456 | 94616247 | 6.45E−31 | −31.09215969 | Hypo-methylated | PCa\|NT DMRs |
| chr4 | 94750894 | 94756007 | 1.66E−05 | 30.42095238 | Hyper-methylated | PCa\|NT DMRs |
| chr4 | 96468588 | 96469265 | 1.92E−05 | 21.86904762 | Hyper-methylated | PCa\|NT DMRs |
| chr4 | 99416600 | 99416658 | 0.00068166 | −24.16369048 | Hypo-methylated | PCa\|NT DMRs |
| chr4 | 99997615 | 100008409 | 3.39E−06 | −34.13779762 | Hypo-methylated | PCa\|NT DMRs |
| chr4 | 100575453 | 100603348 | 0.000125197 | −27.34065934 | Hypo-methylated | PCa\|NT DMRs |
| chr4 | 104458935 | 104524599 | 0.005968871 | −33.07428571 | Hypo-methylated | PCa\|NT DMRs |
| chr4 | 104719128 | 104832121 | 3.82E−07 | −34.40113379 | Hypo-methylated | PCa\|NT DMRs |
| chr4 | 105237730 | 105327614 | 0.001969641 | −30.35396825 | Hypo-methylated | PCa\|NT DMRs |
| chr4 | 107340712 | 107393166 | 0.00064571 | −37.02380952 | Hypo-methylated | PCa\|NT DMRs |
| chr4 | 107958117 | 107958168 | 0.00064571 | 25.77891156 | Hyper-methylated | PCa\|NT DMRs |
| chr4 | 107982224 | 108047704 | 4.16E−07 | −24.87559524 | Hypo-methylated | PCa\|NT DMRs |
| chr4 | 108912031 | 108928173 | 0.002437742 | −36.66402116 | Hypo-methylated | PCa\|NT DMRs |
| chr4 | 110480731 | 110481098 | 8.63E−05 | 21.31819728 | Hyper-methylated | PCa\|NT DMRs |
| chr4 | 111540025 | 111541764 | 1.96E−05 | 20.82571429 | Hyper-methylated | PCa\|NT DMRs |
| chr4 | 111562359 | 111562635 | 8.31E−07 | 25.76466165 | Hyper-methylated | PCa\|NT DMRs |
| chr4 | 113432375 | 113436136 | 0.000216379 | 20.39100529 | Hyper-methylated | PCa\|NT DMRs |
| chr4 | 113630671 | 113650639 | 2.01E−07 | −26.40333333 | Hypo-methylated | PCa\|NT DMRs |
| chr4 | 114823634 | 114823795 | 0.005968871 | −32.71428571 | Hypo-methylated | PCa\|NT DMRs |
| chr4 | 115580142 | 116035083 | 9.00E−07 | −30.6375 | Hypo-methylated | PCa\|NT DMRs |
| chr4 | 120596211 | 120875811 | 4.62E−05 | −23.43015873 | Hypo-methylated | PCa\|NT DMRs |
| chr4 | 123505088 | 123505240 | 0.005968871 | −27.94285714 | Hypo-methylated | PCa\|NT DMRs |
| chr4 | 123747810 | 123747898 | 4.85E−08 | 22.07142857 | Hyper-methylated | PCa\|NT DMRs |
| chr4 | 126239191 | 126539569 | 1.56E−09 | −31.06453202 | Hypo-methylated | PCa\|NT DMRs |
| chr4 | 126692918 | 127411465 | 1.56E−32 | −32.94203297 | Hypo-methylated | PCa\|NT DMRs |
| chr4 | 127591407 | 127794613 | 0.000260936 | −33.26857143 | Hypo-methylated | PCa\|NT DMRs |
| chr4 | 127795836 | 127849482 | 6.17E−06 | −28.40357143 | Hypo-methylated | PCa\|NT DMRs |
| chr4 | 128239783 | 128502727 | 1.29E−07 | −27.00226757 | Hypo-methylated | PCa\|NT DMRs |
| chr4 | 129511106 | 129512806 | 0.001789867 | −28.7989418 | Hypo-methylated | PCa\|NT DMRs |
| chr4 | 130053051 | 130623986 | 0.000406444 | −22.76326531 | Hypo-methylated | PCa\|NT DMRs |
| chr4 | 131416828 | 132385910 | 5.11E−23 | −26.28225108 | Hypo-methylated | PCa\|NT DMRs |
| chr4 | 132898758 | 132959575 | 3.46E−07 | −24.48690476 | Hypo-methylated | PCa\|NT DMRs |
| chr4 | 133107255 | 133488006 | 2.82E−07 | −25.67281746 | Hypo-methylated | PCa\|NT DMRs |
| chr4 | 133730649 | 134029195 | 1.10E−05 | −32.97172619 | Hypo-methylated | PCa\|NT DMRs |
| chr4 | 134301057 | 134306479 | 0.003622801 | −32.23809524 | Hypo-methylated | PCa\|NT DMRs |
| chr4 | 135873891 | 135919809 | 2.86E−05 | −25.12637363 | Hypo-methylated | PCa\|NT DMRs |
| chr4 | 136327930 | 136929593 | 5.45E−13 | −23.97537994 | Hypo-methylated | PCa\|NT DMRs |
| chr4 | 137098530 | 137316687 | 1.91E−18 | −25.55631141 | Hypo-methylated | PCa\|NT DMRs |
| chr4 | 137416378 | 137733900 | 5.48E−17 | −25.82677596 | Hypo-methylated | PCa\|NT DMRs |
| chr4 | 137790035 | 137850577 | 2.86E−05 | −32.6040293 | Hypo-methylated | PCa\|NT DMRs |
| chr4 | 139831807 | 139833697 | 1.97E−05 | −39.67952381 | Hypo-methylated | PCa\|NT DMRs |
| chr4 | 139964244 | 139964408 | 0.001969641 | −53.24206349 | Hypo-methylated | PCa\|NT DMRs |
| chr4 | 140187743 | 140188008 | 0.004095673 | 49.35555556 | Hyper-methylated | PCa\|NT DMRs |
| chr4 | 140554198 | 140555219 | 1.88E−07 | −34.77747748 | Hypo-methylated | PCa\|NT DMRs |
| chr4 | 140656525 | 140686025 | 5.84E−05 | 28.68869048 | Hyper-methylated | PCa\|NT DMRs |
| chr4 | 141489765 | 141489975 | 0.005968871 | 26.17099567 | Hyper-methylated | PCa\|NT DMRs |
| chr4 | 143992360 | 144068518 | 9.23E−05 | −20.30112045 | Hypo-methylated | PCa\|NT DMRs |
| chr4 | 144865812 | 145566787 | 7.88E−11 | −22.33043758 | Hypo-methylated | PCa\|NT DMRs |
| chr4 | 145585398 | 145605621 | 1.21E−07 | −32.4375 | Hypo-methylated | PCa\|NT DMRs |
| chr4 | 146830918 | 146831059 | 0.005968871 | −58.05714286 | Hypo-methylated | PCa\|NT DMRs |
| chr4 | 147164826 | 147165045 | 0.005968871 | 25.94285714 | Hyper-methylated | PCa\|NT DMRs |
| chr4 | 147576548 | 147576614 | 7.19E−05 | 24.01190476 | Hyper-methylated | PCa\|NT DMRs |
| chr4 | 148963047 | 148970731 | 0.00064571 | −41.92176871 | Hypo-methylated | PCa\|NT DMRs |
| chr4 | 152782627 | 152804741 | 0.000836132 | −20.73333333 | Hypo-methylated | PCa\|NT DMRs |
| chr4 | 153600942 | 153601467 | 5.28E−09 | 23.99434524 | Hyper-methylated | PCa\|NT DMRs |
| chr4 | 154386835 | 154386925 | 6.42E−05 | 35.53968254 | Hyper-methylated | PCa\|NT DMRs |
| chr4 | 155444642 | 155444910 | 5.84E−05 | −35.24404762 | Hypo-methylated | PCa\|NT DMRs |
| chr4 | 156933349 | 157120030 | 5.47E−10 | −38.28897243 | Hypo-methylated | PCa\|NT DMRs |
| chr4 | 158175644 | 158557579 | 2.36E−16 | −33.3627794 | Hypo-methylated | PCa\|NT DMRs |
| chr4 | 158740298 | 158914185 | 0.000543976 | −27.44047619 | Hypo-methylated | PCa\|NT DMRs |
| chr4 | 162440444 | 162699172 | 4.22E−19 | −29.30041051 | Hypo-methylated | PCa\|NT DMRs |
| chr4 | 162898581 | 162918857 | 6.42E−05 | −34.8978836 | Hypo-methylated | PCa\|NT DMRs |
| chr4 | 163442605 | 163594802 | 0.00023371 | −36.06380952 | Hypo-methylated | PCa\|NT DMRs |
| chr4 | 164097383 | 164246589 | 3.44E−06 | −40.11041667 | Hypo-methylated | PCa\|NT DMRs |
| chr4 | 164264854 | 164264949 | 2.39E−05 | 22.14880952 | Hyper-methylated | PCa\|NT DMRs |
| chr4 | 167724679 | 167882861 | 2.73E−06 | −26.62857143 | Hypo-methylated | PCa\|NT DMRs |
| chr4 | 169991576 | 169991815 | 0.005968871 | −55.43809524 | Hypo-methylated | PCa\|NT DMRs |
| chr4 | 170695756 | 170696193 | 0.000206041 | 30.95846561 | Hyper-methylated | PCa\|NT DMRs |
| chr4 | 170698920 | 170708508 | 2.42E−05 | −24.39863946 | Hypo-methylated | PCa\|NT DMRs |
| chr4 | 171630877 | 172251746 | 6.08E−36 | −29.58325775 | Hypo-methylated | PCa\|NT DMRs |
| chr4 | 172332382 | 172637033 | 6.16E−06 | −31.00092166 | Hypo-methylated | PCa\|NT DMRs |
| chr4 | 177310888 | 177546393 | 4.30E−08 | −28.99370748 | Hypo-methylated | PCa\|NT DMRs |
| chr4 | 177913924 | 178058918 | 1.57E−07 | −34.27967033 | Hypo-methylated | PCa\|NT DMRs |
| chr4 | 178302966 | 178322402 | 5.19E−07 | −35.1003663 | Hypo-methylated | PCa\|NT DMRs |
| chr4 | 180312144 | 180978557 | 4.85E−13 | −32.65024295 | Hypo-methylated | PCa\|NT DMRs |
| chr4 | 182454200 | 182481924 | 0.005968871 | −28.74285714 | Hypo-methylated | PCa\|NT DMRs |
| chr4 | 183061633 | 183064642 | 1.16E−12 | 29.45541872 | Hyper-methylated | PCa\|NT DMRs |
| chr4 | 183386534 | 183386716 | 0.005968871 | −52.6 | Hypo-methylated | PCa\|NT DMRs |
| chr4 | 183728044 | 183729468 | 3.63E−07 | 25.4262585 | Hyper-methylated | PCa\|NT DMRs |
| chr4 | 183749598 | 183749860 | 2.03E−06 | −29.11714286 | Hypo-methylated | PCa\|NT DMRs |
| chr4 | 184277792 | 184298017 | 1.67E−07 | −30.06961451 | Hypo-methylated | PCa\|NT DMRs |
| chr4 | 184643001 | 184643067 | 0.004095673 | 34.30952381 | Hyper-methylated | PCa\|NT DMRs |
| chr4 | 184675775 | 184681351 | 0.007959104 | −40.31020408 | Hypo-methylated | PCa\|NT DMRs |
| chr4 | 184762268 | 184764025 | 0.000974795 | −37.76428571 | Hypo-methylated | PCa\|NT DMRs |
| chr4 | 184931420 | 184941019 | 0.003122338 | −28.71626984 | Hypo-methylated | PCa\|NT DMRs |
| chr4 | 185110930 | 185138997 | 0.000783791 | −29.43285714 | Hypo-methylated | PCa\|NT DMRs |
| chr4 | 185323926 | 185331194 | 0.004236974 | 39.52428571 | Hyper-methylated | PCa\|NT DMRs |
| chr4 | 185936960 | 185937527 | 3.46E−07 | 32.10337302 | Hyper-methylated | PCa\|NT DMRs |
| chr4 | 186559938 | 186573655 | 2.72E−06 | 38.22196621 | Hyper-methylated | PCa\|NT DMRs |
| chr4 | 186573817 | 186573873 | 0.005968871 | −40.74761905 | Hypo-methylated | PCa\|NT DMRs |
| chr4 | 186954181 | 186961778 | 5.22E−05 | −45.33605442 | Hypo-methylated | PCa\|NT DMRs |
| chr4 | 187218545 | 187218676 | 0.005594179 | −21.64172336 | Hypo-methylated | PCa\|NT DMRs |
| chr4 | 187219383 | 187219649 | 0.001836557 | 25.61538462 | Hyper-methylated | PCa\|NT DMRs |
| chr4 | 187572821 | 187573086 | 2.61E−07 | −49.39047619 | Hypo-methylated | PCa\|NT DMRs |
| chr4 | 188897841 | 188898251 | 0.005968871 | −41.69142857 | Hypo-methylated | PCa\|NT DMRs |
| chr4 | 188953227 | 188953415 | 0.00064571 | 39.06122449 | Hyper-methylated | PCa\|NT DMRs |
| chr4 | 189039289 | 189043369 | 0.001850915 | −29.35357143 | Hypo-methylated | PCa\|NT DMRs |
| chr4 | 189102844 | 189103423 | 0.00064571 | −61.36054422 | Hypo-methylated | PCa\|NT DMRs |
| chr4 | 189204176 | 189204454 | 9.33E−05 | 21.47 | Hyper-methylated | PCa\|NT DMRs |
| chr4 | 189257667 | 189259026 | 0.000481535 | −31.10687831 | Hypo-methylated | PCa\|NT DMRs |
| chr4 | 189390720 | 189433429 | 1.42E−05 | −35.42006803 | Hypo-methylated | PCa\|NT DMRs |
| chr5 | 36644 | 49847 | 0.005968871 | −38.56571429 | Hypo-methylated | PCa\|NT DMRs |
| chr5 | 54484 | 54571 | 0.000205174 | 37.51488095 | Hyper-methylated | PCa\|NT DMRs |
| chr5 | 173045 | 173196 | 0.005513661 | 27.625 | Hyper-methylated | PCa\|NT DMRs |
| chr5 | 191676 | 192133 | 2.77E−06 | 36.03267196 | Hyper-methylated | PCa\|NT DMRs |
| chr5 | 322366 | 323864 | 9.10E−12 | 26.61380145 | Hyper-methylated | PCa\|NT DMRs |
| chr5 | 346708 | 346994 | 0.000339757 | 42.35497835 | Hyper-methylated | PCa\|NT DMRs |
| chr5 | 429311 | 429595 | 0.002373155 | 26.23386243 | Hyper-methylated | PCa\|NT DMRs |
| chr5 | 484994 | 485992 | 1.52E−07 | −33.01020408 | Hypo-methylated | PCa\|NT DMRs |
| chr5 | 486402 | 486528 | 0.000354065 | 25.49206349 | Hyper-methylated | PCa\|NT DMRs |
| chr5 | 491998 | 493316 | 0.000184589 | −21.4272578 | Hypo-methylated | PCa\|NT DMRs |
| chr5 | 496299 | 496580 | 0.000481535 | −32.9015873 | Hypo-methylated | PCa\|NT DMRs |
| chr5 | 504872 | 506563 | 0.000775062 | −26.11564626 | Hypo-methylated | PCa\|NT DMRs |
| chr5 | 510035 | 513353 | 0.009924186 | −20.37142857 | Hypo-methylated | PCa\|NT DMRs |
| chr5 | 524990 | 525799 | 0.004047992 | 20.59013605 | Hyper-methylated | PCa\|NT DMRs |
| chr5 | 528034 | 531433 | 4.46E−17 | 21.77670807 | Hyper-methylated | PCa\|NT DMRs |
| chr5 | 540534 | 541926 | 0.00010546 | −27.95873016 | Hypo-methylated | PCa\|NT DMRs |
| chr5 | 544499 | 544586 | 0.005968871 | 23.34285714 | Hyper-methylated | PCa\|NT DMRs |
| chr5 | 570255 | 574375 | 7.37E−05 | −21.99482402 | Hypo-methylated | PCa\|NT DMRs |
| chr5 | 672237 | 673043 | 0.000911204 | −30.33154762 | Hypo-methylated | PCa\|NT DMRs |
| chr5 | 759101 | 759173 | 0.001969641 | −45.14285714 | Hypo-methylated | PCa\|NT DMRs |
| chr5 | 891730 | 891887 | 0.00064571 | 28.65306122 | Hyper-methylated | PCa\|NT DMRs |
| chr5 | 931118 | 931365 | 0.000481535 | −29.87301587 | Hypo-methylated | PCa\|NT DMRs |
| chr5 | 958837 | 960204 | 9.50E−05 | −46.33643892 | Hypo-methylated | PCa\|NT DMRs |
| chr5 | 982032 | 982752 | 0.003764982 | −25.56904762 | Hypo-methylated | PCa\|NT DMRs |
| chr5 | 1101971 | 1102748 | 0.003034084 | 25.38027211 | Hyper-methylated | PCa\|NT DMRs |
| chr5 | 1106174 | 1109475 | 6.30E−16 | 34.92054016 | Hyper-methylated | PCa\|NT DMRs |
| chr5 | 1130994 | 1131377 | 6.69E−05 | −21.97714286 | Hypo-methylated | PCa\|NT DMRs |
| chr5 | 1161973 | 1162279 | 6.42E−05 | −41.11111111 | Hypo-methylated | PCa\|NT DMRs |
| chr5 | 1201147 | 1202000 | 1.15E−06 | −29.14115004 | Hypo-methylated | PCa\|NT DMRs |
| chr5 | 1245835 | 1245903 | 0.001969641 | −28.78571429 | Hypo-methylated | PCa\|NT DMRs |
| chr5 | 1282489 | 1282797 | 6.34E−06 | −25.92857143 | Hypo-methylated | PCa\|NT DMRs |
| chr5 | 1408867 | 1409169 | 2.05E−05 | 36.5974026 | Hyper-methylated | PCa\|NT DMRs |
| chr5 | 1493248 | 1497619 | 0.001821096 | 21.92431973 | Hyper-methylated | PCa\|NT DMRs |
| chr5 | 1593020 | 1594436 | 2.69E−06 | 23.16300366 | Hyper-methylated | PCa\|NT DMRs |
| chr5 | 1617871 | 1620046 | 0.000226073 | 20.28428571 | Hyper-methylated | PCa\|NT DMRs |
| chr5 | 1642528 | 1643655 | 8.96E−05 | −24.6452381 | Hypo-methylated | PCa\|NT DMRs |
| chr5 | 1767836 | 1768211 | 0.002770271 | −22.2967033 | Hypo-methylated | PCa\|NT DMRs |
| chr5 | 1770678 | 1772639 | 6.21E−07 | −41.0789916 | Hypo-methylated | PCa\|NT DMRs |
| chr5 | 1787184 | 1787751 | 0.003871035 | −34.24702381 | Hypo-methylated | PCa\|NT DMRs |
| chr5 | 1800475 | 1800685 | 5.02E−05 | 21.31092437 | Hyper-methylated | PCa\|NT DMRs |
| chr5 | 1841249 | 1841681 | 1.52E−07 | 39.65884354 | Hyper-methylated | PCa\|NT DMRs |
| chr5 | 1887890 | 1888331 | 1.33E−06 | 24.66666667 | Hyper-methylated | PCa\|NT DMRs |
| chr5 | 1912091 | 1914239 | 4.08E−06 | −23.68005952 | Hypo-methylated | PCa\|NT DMRs |
| chr5 | 1947200 | 1950439 | 4.18E−07 | −33.49062049 | Hypo-methylated | PCa\|NT DMRs |
| chr5 | 1952403 | 1953067 | 6.94E−06 | 32.30539683 | Hyper-methylated | PCa\|NT DMRs |
| chr5 | 2007414 | 2008029 | 1.65E−08 | −33.48467262 | Hypo-methylated | PCa\|NT DMRs |
| chr5 | 2627370 | 2635841 | 0.000554289 | −29.82301587 | Hypo-methylated | PCa\|NT DMRs |
| chr5 | 2646033 | 2657115 | 0.004149771 | −29.01978022 | Hypo-methylated | PCa\|NT DMRs |
| chr5 | 2737144 | 2739365 | 9.98E−10 | 30.8673774 | Hyper-methylated | PCa\|NT DMRs |
| chr5 | 5320280 | 5320485 | 0.00064571 | −45.55102041 | Hypo-methylated | PCa\|NT DMRs |
| chr5 | 5688533 | 5688867 | 0.00068166 | 28.1452381 | Hyper-methylated | PCa\|NT DMRs |
| chr5 | 5887486 | 5954559 | 9.84E−07 | −28.18272109 | Hypo-methylated | PCa\|NT DMRs |
| chr5 | 6583091 | 6583897 | 3.60E−15 | 30.54106167 | Hyper-methylated | PCa\|NT DMRs |
| chr5 | 6999237 | 7002383 | 0.005968871 | −27.66190476 | Hypo-methylated | PCa\|NT DMRs |
| chr5 | 7873542 | 7873602 | 0.004057568 | 23.42857143 | Hyper-methylated | PCa\|NT DMRs |
| chr5 | 7891828 | 8429773 | 1.26E−18 | −29.71930855 | Hypo-methylated | PCa\|NT DMRs |
| chr5 | 8822315 | 8839953 | 0.001969641 | −42.53571429 | Hypo-methylated | PCa\|NT DMRs |
| chr5 | 9090845 | 9328923 | 2.49E−08 | −27.57619048 | Hypo-methylated | PCa\|NT DMRs |
| chr5 | 9546424 | 9546840 | 6.10E−08 | 28.60057143 | Hyper-methylated | PCa\|NT DMRs |
| chr5 | 9606704 | 10065719 | 1.08E−12 | −28.94210526 | Hypo-methylated | PCa\|NT DMRs |
| chr5 | 10080596 | 10083348 | 0.000111427 | −29.24285714 | Hypo-methylated | PCa\|NT DMRs |
| chr5 | 10466910 | 10467001 | 0.005968871 | 50.00952381 | Hyper-methylated | PCa\|NT DMRs |
| chr5 | 10519079 | 10519314 | 0.005968871 | −34.45714286 | Hypo-methylated | PCa\|NT DMRs |
| chr5 | 10561931 | 10563665 | 1.74E−06 | 31.00625 | Hyper-methylated | PCa\|NT DMRs |
| chr5 | 10625692 | 10632701 | 0.003391914 | 27.38624339 | Hyper-methylated | PCa\|NT DMRs |
| chr5 | 10638145 | 10650090 | 1.50E−10 | −30.05619048 | Hypo-methylated | PCa\|NT DMRs |
| chr5 | 10761664 | 10762290 | 6.95E−05 | 30.00952381 | Hyper-methylated | PCa\|NT DMRs |
| chr5 | 12574955 | 12590482 | 0.001969641 | −23.98968254 | Hypo-methylated | PCa\|NT DMRs |
| chr5 | 12664526 | 12777965 | 6.67E−06 | −22.53303571 | Hypo-methylated | PCa\|NT DMRs |
| chr5 | 12805449 | 13466472 | 4.39E−16 | −33.2144528 | Hypo-methylated | PCa\|NT DMRs |
| chr5 | 13579536 | 13642747 | 3.77E−05 | −22.21507937 | Hypo-methylated | PCa\|NT DMRs |
| chr5 | 14131700 | 14132687 | 0.003464153 | −22.24242424 | Hypo-methylated | PCa\|NT DMRs |
| chr5 | 16215940 | 16226414 | 0.001969641 | −33.90873016 | Hypo-methylated | PCa\|NT DMRs |
| chr5 | 16740672 | 16740820 | 0.00064571 | 37.93877551 | Hyper-methylated | PCa\|NT DMRs |
| chr5 | 17053075 | 17061084 | 0.007959104 | −38.52789116 | Hypo-methylated | PCa\|NT DMRs |
| chr5 | 17218716 | 17219330 | 0.000575877 | 29.7965368 | Hyper-methylated | PCa\|NT DMRs |
| chr5 | 17506966 | 17620988 | 0.001775064 | −27.71190476 | Hypo-methylated | PCa\|NT DMRs |
| chr5 | 18113703 | 18291499 | 6.42E−05 | −34.58201058 | Hypo-methylated | PCa\|NT DMRs |
| chr5 | 18468601 | 18701383 | 5.91E−05 | −24.67981859 | Hypo-methylated | PCa\|NT DMRs |
| chr5 | 18754451 | 18837250 | 7.28E−07 | −32.2556391 | Hypo-methylated | PCa\|NT DMRs |
| chr5 | 18902785 | 19105793 | 2.27E−11 | −35.88246753 | Hypo-methylated | PCa\|NT DMRs |
| chr5 | 19185189 | 19360890 | 3.12E−11 | −30.36013289 | Hypo-methylated | PCa\|NT DMRs |
| chr5 | 19630207 | 19687332 | 0.000398029 | −29.36263736 | Hypo-methylated | PCa\|NT DMRs |
| chr5 | 20693877 | 21044402 | 7.31E−05 | −28.2755102 | Hypo-methylated | PCa\|NT DMRs |
| chr5 | 21876551 | 21935096 | 6.42E−05 | −34.72698413 | Hypo-methylated | PCa\|NT DMRs |
| chr5 | 23179652 | 23411522 | 2.52E−13 | −33.49488966 | Hypo-methylated | PCa\|NT DMRs |
| chr5 | 24318343 | 24433204 | 2.38E−07 | −29.10497835 | Hypo-methylated | PCa\|NT DMRs |
| chr5 | 24706900 | 25154479 | 1.28E−12 | −36.04371184 | Hypo-methylated | PCa\|NT DMRs |
| chr5 | 25190742 | 25200415 | 4.05E−15 | −25.99439153 | Hypo-methylated | PCa\|NT DMRs |
| chr5 | 25710410 | 26030670 | 1.56E−18 | −33.20130568 | Hypo-methylated | PCa\|NT DMRs |
| chr5 | 26145538 | 26229422 | 1.23E−07 | −33.65833333 | Hypo-methylated | PCa\|NT DMRs |
| chr5 | 26324500 | 26468493 | 6.33E−13 | −31.26825397 | Hypo-methylated | PCa\|NT DMRs |
| chr5 | 26704766 | 27230652 | 2.88E−25 | −37.77136978 | Hypo-methylated | PCa\|NT DMRs |
| chr5 | 27267583 | 27408078 | 0.000145366 | −34.05204082 | Hypo-methylated | PCa\|NT DMRs |
| chr5 | 27493792 | 27783717 | 3.09E−13 | −26.32439261 | Hypo-methylated | PCa\|NT DMRs |
| chr5 | 28575715 | 28747075 | 3.19E−07 | −28.87929607 | Hypo-methylated | PCa\|NT DMRs |
| chr5 | 28928178 | 28928431 | 3.62E−08 | 21.83669468 | Hyper-methylated | PCa\|NT DMRs |
| chr5 | 30139519 | 30169473 | 0.000377317 | −32.99107143 | Hypo-methylated | PCa\|NT DMRs |
| chr5 | 30345913 | 30346846 | 2.66E−05 | −25.69546485 | Hypo-methylated | PCa\|NT DMRs |
| chr5 | 30935659 | 31182658 | 8.01E−11 | −29.88404762 | Hypo-methylated | PCa\|NT DMRs |
| chr5 | 31212521 | 31339254 | 2.06E−05 | −36.32823129 | Hypo-methylated | PCa\|NT DMRs |
| chr5 | 31639750 | 31640087 | 8.92E−06 | 30.04004329 | Hyper-methylated | PCa\|NT DMRs |
| chr5 | 32128621 | 32128689 | 0.00064571 | −28.04081633 | Hypo-methylated | PCa\|NT DMRs |
| chr5 | 32587995 | 32613011 | 0.000564835 | −29.72142857 | Hypo-methylated | PCa\|NT DMRs |
| chr5 | 33891748 | 33892458 | 1.18E−08 | 37.00416667 | Hyper-methylated | PCa\|NT DMRs |
| chr5 | 37834171 | 37836849 | 6.90E−10 | 22.48412698 | Hyper-methylated | PCa\|NT DMRs |
| chr5 | 37838564 | 37839313 | 2.73E−05 | 22.02756892 | Hyper-methylated | PCa\|NT DMRs |
| chr5 | 38016960 | 38017179 | 0.001969641 | −20.28571429 | Hypo-methylated | PCa\|NT DMRs |
| chr5 | 38257858 | 38258134 | 5.19E−07 | 33.73699634 | Hyper-methylated | PCa\|NT DMRs |
| chr5 | 40930560 | 41204864 | 2.07E−09 | −29.95608466 | Hypo-methylated | PCa\|NT DMRs |
| chr5 | 41520375 | 41520434 | 0.004947537 | −32.67936508 | Hypo-methylated | PCa\|NT DMRs |
| chr5 | 42756739 | 42756905 | 0.003622801 | 29.21031746 | Hyper-methylated | PCa\|NT DMRs |
| chr5 | 43603430 | 43604298 | 1.16E−11 | 27.46957027 | Hyper-methylated | PCa\|NT DMRs |
| chr5 | 43745994 | 43825042 | 3.61E−19 | −27.48801843 | Hypo-methylated | PCa\|NT DMRs |
| chr5 | 44209423 | 44209492 | 0.005968871 | −33.97142857 | Hypo-methylated | PCa\|NT DMRs |
| chr5 | 44483467 | 44559127 | 6.23E−06 | −32.41077694 | Hypo-methylated | PCa\|NT DMRs |
| chr5 | 44847360 | 44862657 | 0.005968871 | −41.48571429 | Hypo-methylated | PCa\|NT DMRs |
| chr5 | 45889683 | 45891129 | 0.003764982 | −21.58611111 | Hypo-methylated | PCa\|NT DMRs |
| chr5 | 49971395 | 49971443 | 0.005968871 | −48.74285714 | Hypo-methylated | PCa\|NT DMRs |
| chr5 | 50260158 | 50264788 | 5.12E−08 | 33.44543651 | Hyper-methylated | PCa\|NT DMRs |
| chr5 | 50699155 | 50925915 | 0.002097193 | −38.13605442 | Hypo-methylated | PCa\|NT DMRs |
| chr5 | 51175685 | 51257655 | 6.50E−05 | −26.44312169 | Hypo-methylated | PCa\|NT DMRs |
| chr5 | 52096913 | 52097499 | 0.001451132 | −36.37142857 | Hypo-methylated | PCa\|NT DMRs |
| chr5 | 52778621 | 52778799 | 0.003949155 | 41.11507937 | Hyper-methylated | PCa\|NT DMRs |
| chr5 | 53941842 | 53942290 | 0.008482405 | 46.34285714 | Hyper-methylated | PCa\|NT DMRs |
| chr5 | 54530301 | 54537833 | 0.003622801 | −35.33333333 | Hypo-methylated | PCa\|NT DMRs |
| chr5 | 57859560 | 57859666 | 5.83E−05 | −22.65833333 | Hypo-methylated | PCa\|NT DMRs |
| chr5 | 58423471 | 58423726 | 0.000468799 | 47.71428571 | Hyper-methylated | PCa\|NT DMRs |
| chr5 | 64548943 | 64754400 | 1.57E−06 | −20.90993789 | Hypo-methylated | PCa\|NT DMRs |
| chr5 | 66461444 | 66504456 | 2.85E−11 | 29.43103448 | Hyper-methylated | PCa\|NT DMRs |
| chr5 | 67482957 | 67483406 | 0.000377317 | −29.425 | Hypo-methylated | PCa\|NT DMRs |
| chr5 | 68339298 | 68363946 | 0.005339316 | −22.95442177 | Hypo-methylated | PCa\|NT DMRs |
| chr5 | 70979930 | 71015616 | 0.009788405 | 23.55442177 | Hyper-methylated | PCa\|NT DMRs |
| chr5 | 71234676 | 71235006 | 0.005968871 | −45.28285714 | Hypo-methylated | PCa\|NT DMRs |
| chr5 | 72731939 | 72732636 | 6.92E−15 | 27.50103896 | Hyper-methylated | PCa\|NT DMRs |
| chr5 | 72740460 | 72740771 | 0.000111427 | 29.60238095 | Hyper-methylated | PCa\|NT DMRs |
| chr5 | 74162557 | 74162881 | 0.000254585 | 29.04166667 | Hyper-methylated | PCa\|NT DMRs |
| chr5 | 75013592 | 75013683 | 0.004095673 | −22.10714286 | Hypo-methylated | PCa\|NT DMRs |
| chr5 | 75377487 | 75378064 | 0.000199784 | 27.05488722 | Hyper-methylated | PCa\|NT DMRs |
| chr5 | 76933448 | 76940450 | 1.61E−24 | 22.14285714 | Hyper-methylated | PCa\|NT DMRs |
| chr5 | 77268417 | 77270020 | 1.00E−10 | 26.12254616 | Hyper-methylated | PCa\|NT DMRs |
| chr5 | 77943296 | 77943461 | 0.003622801 | 41.11904762 | Hyper-methylated | PCa\|NT DMRs |
| chr5 | 78984891 | 78985871 | 2.89E−15 | −42.11079932 | Hypo-methylated | PCa\|NT DMRs |
| chr5 | 79167786 | 79169828 | 0.003949155 | −25.10714286 | Hypo-methylated | PCa\|NT DMRs |
| chr5 | 80528526 | 80528737 | 0.00641359 | −24.14285714 | Hypo-methylated | PCa\|NT DMRs |
| chr5 | 82155603 | 82155778 | 0.00064571 | −41.51020408 | Hypo-methylated | PCa\|NT DMRs |
| chr5 | 83024021 | 83642055 | 1.93E−13 | −32.61940035 | Hypo-methylated | PCa\|NT DMRs |
| chr5 | 85756620 | 85865687 | 2.32E−10 | −29.24831933 | Hypo-methylated | PCa\|NT DMRs |
| chr5 | 90794947 | 90795417 | 0.001181579 | −35.24421769 | Hypo-methylated | PCa\|NT DMRs |
| chr5 | 91030006 | 91244923 | 4.10E−09 | −31.82126984 | Hypo-methylated | PCa\|NT DMRs |
| chr5 | 92346121 | 92521069 | 0.005968871 | −24.07619048 | Hypo-methylated | PCa\|NT DMRs |
| chr5 | 94955700 | 94955935 | 0.005968871 | 34.44666667 | Hyper-methylated | PCa\|NT DMRs |
| chr5 | 95159826 | 95163055 | 0.003448944 | 41.57346939 | Hyper-methylated | PCa\|NT DMRs |
| chr5 | 95295896 | 95296042 | 0.000972067 | −29.4047619 | Hypo-methylated | PCa\|NT DMRs |
| chr5 | 98420562 | 98695197 | 6.98E−05 | −28.56938776 | Hypo-methylated | PCa\|NT DMRs |
| chr5 | 98909608 | 99401805 | 3.77E−14 | −30.68787062 | Hypo-methylated | PCa\|NT DMRs |
| chr5 | 101373974 | 101463725 | 6.42E−05 | −34.59470899 | Hypo-methylated | PCa\|NT DMRs |
| chr5 | 101827404 | 101941754 | 5.01E−08 | −32.71172161 | Hypo-methylated | PCa\|NT DMRs |
| chr5 | 103074137 | 103100319 | 0.000117699 | −29.67662338 | Hypo-methylated | PCa\|NT DMRs |
| chr5 | 103156025 | 103285997 | 1.15E−06 | −26.89484127 | Hypo-methylated | PCa\|NT DMRs |
| chr5 | 104269547 | 104450770 | 0.00064571 | −27.0170068 | Hypo-methylated | PCa\|NT DMRs |
| chr5 | 105554186 | 105582068 | 4.90E−08 | −26.07467532 | Hypo-methylated | PCa\|NT DMRs |
| chr5 | 105954399 | 106038183 | 0.000118733 | −32.21746032 | Hypo-methylated | PCa\|NT DMRs |
| chr5 | 107778055 | 107895031 | 0.000145094 | −27.28571429 | Hypo-methylated | PCa\|NT DMRs |
| chr5 | 109468813 | 109883511 | 4.09E−06 | −24.74421769 | Hypo-methylated | PCa\|NT DMRs |
| chr5 | 110948125 | 110953963 | 0.000339627 | −29.92420635 | Hypo-methylated | PCa\|NT DMRs |
| chr5 | 111194066 | 111440748 | 3.43E−06 | −37.04017857 | Hypo-methylated | PCa\|NT DMRs |
| chr5 | 112373361 | 112379442 | 0.001789867 | −26.14021164 | Hypo-methylated | PCa\|NT DMRs |
| chr5 | 115912116 | 115912302 | 7.75E−06 | 21.84033613 | Hyper-methylated | PCa\|NT DMRs |
| chr5 | 121187787 | 121266661 | 2.97E−11 | −24.61230159 | Hypo-methylated | PCa\|NT DMRs |
| chr5 | 122620950 | 122621646 | 4.33E−05 | 35.88278388 | Hyper-methylated | PCa\|NT DMRs |
| chr5 | 124079870 | 124079990 | 0.00064571 | 30.84761905 | Hyper-methylated | PCa\|NT DMRs |
| chr5 | 125259773 | 125259951 | 0.00179283 | −28.35595238 | Hypo-methylated | PCa\|NT DMRs |
| chr5 | 125937484 | 125937691 | 0.000392715 | −30.21428571 | Hypo-methylated | PCa\|NT DMRs |
| chr5 | 126204968 | 126205018 | 6.20E−05 | 43.96938776 | Hyper-methylated | PCa\|NT DMRs |
| chr5 | 129116030 | 129204213 | 0.00641359 | −27.14285714 | Hypo-methylated | PCa\|NT DMRs |
| chr5 | 132602532 | 132613572 | 0.00064571 | 20.40204082 | Hyper-methylated | PCa\|NT DMRs |
| chr5 | 132830468 | 132830845 | 0.000974795 | −41.42559524 | Hypo-methylated | PCa\|NT DMRs |
| chr5 | 133264423 | 133264557 | 0.008482405 | 34.31428571 | Hyper-methylated | PCa\|NT DMRs |
| chr5 | 133451049 | 133477329 | 2.40E−06 | 21.32251701 | Hyper-methylated | PCa\|NT DMRs |
| chr5 | 133513536 | 133513690 | 0.005968871 | −46.71428571 | Hypo-methylated | PCa\|NT DMRs |
| chr5 | 133890165 | 133905232 | 0.001238355 | −30.08809524 | Hypo-methylated | PCa\|NT DMRs |
| chr5 | 134009546 | 134016320 | 0.003622801 | −35.85714286 | Hypo-methylated | PCa\|NT DMRs |
| chr5 | 134713990 | 134714234 | 0.005968871 | 46.62380952 | Hyper-methylated | PCa\|NT DMRs |
| chr5 | 134735440 | 134735756 | 0.003871035 | 31.54702381 | Hyper-methylated | PCa\|NT DMRs |
| chr5 | 134818134 | 134823833 | 0.001583951 | 27.58095238 | Hyper-methylated | PCa\|NT DMRs |
| chr5 | 135415093 | 135415259 | 3.32E−06 | 32.56507937 | Hyper-methylated | PCa\|NT DMRs |
| chr5 | 137225015 | 137225102 | 6.94E−06 | 27.58095238 | Hyper-methylated | PCa\|NT DMRs |
| chr5 | 137794151 | 137795306 | 6.94E−06 | 48.03428571 | Hyper-methylated | PCa\|NT DMRs |
| chr5 | 138849588 | 138852391 | 0.002654087 | 32.89115646 | Hyper-methylated | PCa\|NT DMRs |
| chr5 | 138855434 | 138870698 | 0.004172333 | 25.32912088 | Hyper-methylated | PCa\|NT DMRs |
| chr5 | 138923154 | 138923271 | 0.00064571 | 44.50340136 | Hyper-methylated | PCa\|NT DMRs |
| chr5 | 139026562 | 139027511 | 1.42E−05 | 28.85504762 | Hyper-methylated | PCa\|NT DMRs |
| chr5 | 139127989 | 139128275 | 0.000632596 | 28.90606061 | Hyper-methylated | PCa\|NT DMRs |
| chr5 | 139284247 | 139284813 | 0.000153204 | 20.28571429 | Hyper-methylated | PCa\|NT DMRs |
| chr5 | 139303814 | 139304570 | 8.96E−05 | −29.08333333 | Hypo-methylated | PCa\|NT DMRs |
| chr5 | 139525949 | 139528263 | 3.38E−07 | 48.17391304 | Hyper-methylated | PCa\|NT DMRs |
| chr5 | 139532389 | 139533233 | 0.005513661 | 28.66938776 | Hyper-methylated | PCa\|NT DMRs |
| chr5 | 140098496 | 140099544 | 5.94E−06 | −43.77186147 | Hypo-methylated | PCa\|NT DMRs |
| chr5 | 140347000 | 140347544 | 0.005033773 | 22.76785714 | Hyper-methylated | PCa\|NT DMRs |
| chr5 | 140723450 | 140723770 | 0.004422055 | 21.58095238 | Hyper-methylated | PCa\|NT DMRs |
| chr5 | 140892917 | 140936423 | 0.000328592 | 32.02517007 | Hyper-methylated | PCa\|NT DMRs |
| chr5 | 141062050 | 141065916 | 0.000133092 | 36.92202381 | Hyper-methylated | PCa\|NT DMRs |
| chr5 | 141325409 | 141330954 | 0.001969641 | 32.31904762 | Hyper-methylated | PCa\|NT DMRs |
| chr5 | 141337511 | 141338045 | 0.00064571 | 40.27687075 | Hyper-methylated | PCa\|NT DMRs |
| chr5 | 141675101 | 141675185 | 0.000392715 | 28.33809524 | Hyper-methylated | PCa\|NT DMRs |
| chr5 | 141703419 | 141703630 | 0.000205174 | 43.92857143 | Hyper-methylated | PCa\|NT DMRs |
| chr5 | 141737215 | 141737877 | 0.000335989 | 32.86520147 | Hyper-methylated | PCa\|NT DMRs |
| chr5 | 141931182 | 141931551 | 5.19E−07 | 35.09340659 | Hyper-methylated | PCa\|NT DMRs |
| chr5 | 141980430 | 141980603 | 0.001969641 | −51.40634921 | Hypo-methylated | PCa\|NT DMRs |
| chr5 | 142563071 | 142563232 | 0.008482405 | 47.60952381 | Hyper-methylated | PCa\|NT DMRs |
| chr5 | 145099211 | 145132848 | 0.001181579 | −26.26530612 | Hypo-methylated | PCa\|NT DMRs |
| chr5 | 145214426 | 145214485 | 0.00023371 | 21.44285714 | Hyper-methylated | PCa\|NT DMRs |
| chr5 | 145235917 | 145236069 | 0.002097193 | 32.66122449 | Hyper-methylated | PCa\|NT DMRs |
| chr5 | 146302496 | 146493322 | 4.48E−06 | −26.44738095 | Hypo-methylated | PCa\|NT DMRs |
| chr5 | 148463858 | 148505346 | 0.009069241 | −36.68398268 | Hypo-methylated | PCa\|NT DMRs |
| chr5 | 148610935 | 148620254 | 0.005513661 | 41.85714286 | Hyper-methylated | PCa\|NT DMRs |
| chr5 | 148678581 | 148679101 | 1.12E−06 | 44.89021164 | Hyper-methylated | PCa\|NT DMRs |
| chr5 | 148762553 | 148784809 | 0.005968871 | 39.57142857 | Hyper-methylated | PCa\|NT DMRs |
| chr5 | 149010096 | 149010377 | 0.002097193 | −28.23809524 | Hypo-methylated | PCa\|NT DMRs |
| chr5 | 149020890 | 149033180 | 0.001969641 | −54.88095238 | Hypo-methylated | PCa\|NT DMRs |
| chr5 | 149056968 | 149067470 | 0.004350823 | 40.47619048 | Hyper-methylated | PCa\|NT DMRs |
| chr5 | 149312425 | 149312649 | 0.000114675 | −32.92857143 | Hypo-methylated | PCa\|NT DMRs |
| chr5 | 149319932 | 149324152 | 0.004537864 | 26.81318681 | Hyper-methylated | PCa\|NT DMRs |
| chr5 | 149495250 | 149495624 | 0.000339757 | 27.84199134 | Hyper-methylated | PCa\|NT DMRs |
| chr5 | 149507322 | 149512117 | 0.007679542 | −20.4984127 | Hypo-methylated | PCa\|NT DMRs |
| chr5 | 149546247 | 149546970 | 1.09E−12 | 27.17352381 | Hyper-methylated | PCa\|NT DMRs |
| chr5 | 149680272 | 149682357 | 7.47E−08 | 28.3971686 | Hyper-methylated | PCa\|NT DMRs |
| chr5 | 149788811 | 149792331 | 0.00090274 | 22.56428571 | Hyper-methylated | PCa\|NT DMRs |
| chr5 | 150226252 | 150226355 | 0.00064571 | 54.37755102 | Hyper-methylated | PCa\|NT DMRs |
| chr5 | 150381248 | 150400461 | 7.67E−07 | 29.93471582 | Hyper-methylated | PCa\|NT DMRs |
| chr5 | 150461339 | 150480143 | 0.003921091 | 39.46493506 | Hyper-methylated | PCa\|NT DMRs |
| chr5 | 150535926 | 150537241 | 2.49E−05 | 34.22811791 | Hyper-methylated | PCa\|NT DMRs |
| chr5 | 150701672 | 150703337 | 0.00068166 | −22.02083333 | Hypo-methylated | PCa\|NT DMRs |
| chr5 | 150759836 | 150775766 | 0.000652677 | −20.83134921 | Hypo-methylated | PCa\|NT DMRs |
| chr5 | 151066304 | 151066713 | 1.42E−05 | 36.95918367 | Hyper-methylated | PCa\|NT DMRs |
| chr5 | 151149074 | 151150352 | 0.007305013 | 22.89285714 | Hyper-methylated | PCa\|NT DMRs |
| chr5 | 151205902 | 151261495 | 0.005685126 | −22.72857143 | Hypo-methylated | PCa\|NT DMRs |
| chr5 | 151355980 | 151540167 | 0.000118733 | −28.80952381 | Hypo-methylated | PCa\|NT DMRs |
| chr5 | 153872888 | 153873228 | 4.26E−06 | 30.43736264 | Hyper-methylated | PCa\|NT DMRs |
| chr5 | 154026679 | 154027124 | 1.47E−07 | 26.29251701 | Hyper-methylated | PCa\|NT DMRs |
| chr5 | 154318209 | 154320377 | 0.007347803 | −24.31746032 | Hypo-methylated | PCa\|NT DMRs |
| chr5 | 157038194 | 157058698 | 0.000117507 | −23.5025974 | Hypo-methylated | PCa\|NT DMRs |
| chr5 | 157464019 | 157464759 | 7.33E−05 | −22.34063492 | Hypo-methylated | PCa\|NT DMRs |
| chr5 | 158478379 | 158478594 | 1.93E−10 | 28.34047619 | Hyper-methylated | PCa\|NT DMRs |
| chr5 | 158523996 | 158526005 | 3.00E−08 | 29.16324405 | Hyper-methylated | PCa\|NT DMRs |
| chr5 | 158787806 | 158787872 | 0.004095673 | 28.38095238 | Hyper-methylated | PCa\|NT DMRs |
| chr5 | 159560571 | 159560827 | 0.003093471 | 25.09285714 | Hyper-methylated | PCa\|NT DMRs |
| chr5 | 162998919 | 163318501 | 1.86E−05 | −28.92889984 | Hypo-methylated | PCa\|NT DMRs |
| chr5 | 167715273 | 167715544 | 6.42E−05 | −40.93386243 | Hypo-methylated | PCa\|NT DMRs |
| chr5 | 167775197 | 167782355 | 0.000176046 | −45.58008658 | Hypo-methylated | PCa\|NT DMRs |
| chr5 | 167836808 | 167836882 | 0.005968871 | 23.28571429 | Hyper-methylated | PCa\|NT DMRs |
| chr5 | 168726818 | 168726890 | 0.00023371 | 37.19761905 | Hyper-methylated | PCa\|NT DMRs |
| chr5 | 168801252 | 168919140 | 0.000443725 | −27.61111111 | Hypo-methylated | PCa\|NT DMRs |
| chr5 | 170877505 | 170878722 | 5.13E−14 | 27.43551165 | Hyper-methylated | PCa\|NT DMRs |
| chr5 | 171023322 | 171023827 | 5.99E−06 | −21.57857143 | Hypo-methylated | PCa\|NT DMRs |
| chr5 | 171531198 | 171531314 | 0.005968871 | 50.60380952 | Hyper-methylated | PCa\|NT DMRs |
| chr5 | 171808412 | 171808618 | 0.001969641 | −36.37301587 | Hypo-methylated | PCa\|NT DMRs |
| chr5 | 171880789 | 171880984 | 0.00068166 | 28.56309524 | Hyper-methylated | PCa\|NT DMRs |
| chr5 | 171997039 | 172013458 | 0.001739875 | −25.56712018 | Hypo-methylated | PCa\|NT DMRs |
| chr5 | 172068912 | 172070257 | 0.000564835 | 27.84139194 | Hyper-methylated | PCa\|NT DMRs |
| chr5 | 172087043 | 172087243 | 0.001181579 | 29.42176871 | Hyper-methylated | PCa\|NT DMRs |
| chr5 | 172110584 | 172111500 | 9.51E−36 | 32.04674759 | Hyper-methylated | PCa\|NT DMRs |
| chr5 | 172227221 | 172234494 | 0.000444042 | 27.75465839 | Hyper-methylated | PCa\|NT DMRs |
| chr5 | 172300082 | 172305014 | 0.00013674 | 29.70833333 | Hyper-methylated | PCa\|NT DMRs |
| chr5 | 172327210 | 172335891 | 8.62E−10 | −28.2018759 | Hypo-methylated | PCa\|NT DMRs |
| chr5 | 172692358 | 172696125 | 0.004505524 | −20.71626984 | Hypo-methylated | PCa\|NT DMRs |
| chr5 | 172754083 | 172754173 | 1.43E−05 | 25.79411765 | Hyper-methylated | PCa\|NT DMRs |
| chr5 | 172756806 | 172757075 | 0.003218673 | 26.61538462 | Hyper-methylated | PCa\|NT DMRs |
| chr5 | 173191590 | 173193936 | 0.000205174 | −39.85178571 | Hypo-methylated | PCa\|NT DMRs |
| chr5 | 173291337 | 173293672 | 0.000570576 | 31.74126984 | Hyper-methylated | PCa\|NT DMRs |
| chr5 | 173317638 | 173317861 | 0.005968871 | −49.3952381 | Hypo-methylated | PCa\|NT DMRs |
| chr5 | 173533377 | 173593695 | 5.23E−08 | −27.71128748 | Hypo-methylated | PCa\|NT DMRs |
| chr5 | 173734760 | 173738678 | 0.00922177 | −26.49421769 | Hypo-methylated | PCa\|NT DMRs |
| chr5 | 173738735 | 173738778 | 0.005968871 | 27.72857143 | Hyper-methylated | PCa\|NT DMRs |
| chr5 | 173814296 | 173834459 | 0.001789867 | −27.86772487 | Hypo-methylated | PCa\|NT DMRs |
| chr5 | 173850763 | 173872341 | 0.000443725 | −30.70198413 | Hypo-methylated | PCa\|NT DMRs |
| chr5 | 173979531 | 174033581 | 0.00010287 | −33.32142857 | Hypo-methylated | PCa\|NT DMRs |
| chr5 | 174035973 | 174036107 | 0.004095673 | 26.02380952 | Hyper-methylated | PCa\|NT DMRs |
| chr5 | 174158681 | 174160942 | 9.64E−21 | 24.02464986 | Hyper-methylated | PCa\|NT DMRs |
| chr5 | 174643420 | 174762038 | 0.000286238 | −26.3697619 | Hypo-methylated | PCa\|NT DMRs |
| chr5 | 175056061 | 175069058 | 0.000205174 | −31.95238095 | Hypo-methylated | PCa\|NT DMRs |
| chr5 | 175085419 | 175085716 | 1.12E−06 | 21.8015873 | Hyper-methylated | PCa\|NT DMRs |
| chr5 | 175136593 | 175155598 | 5.36E−06 | −35.33452381 | Hypo-methylated | PCa\|NT DMRs |
| chr5 | 175200325 | 175200399 | 0.00023371 | 21.18571429 | Hyper-methylated | PCa\|NT DMRs |
| chr5 | 175604239 | 175614465 | 0.003314163 | −23.74285714 | Hypo-methylated | PCa\|NT DMRs |
| chr5 | 175793371 | 175793409 | 0.006096657 | −20.25 | Hypo-methylated | PCa\|NT DMRs |
| chr5 | 175956550 | 175963212 | 0.000130675 | 22.02559524 | Hyper-methylated | PCa\|NT DMRs |
| chr5 | 175995834 | 176002739 | 2.60E−05 | −23.83571429 | Hypo-methylated | PCa\|NT DMRs |
| chr5 | 176008274 | 176008316 | 0.005968871 | −24.69142857 | Hypo-methylated | PCa\|NT DMRs |
| chr5 | 176153803 | 176154061 | 8.36E−06 | 46.12328042 | Hyper-methylated | PCa\|NT DMRs |
| chr5 | 176163639 | 176166571 | 0.005526313 | 20.83968254 | Hyper-methylated | PCa\|NT DMRs |
| chr5 | 176170751 | 176171320 | 4.10E−09 | 26.02761905 | Hyper-methylated | PCa\|NT DMRs |
| chr5 | 176212655 | 176213169 | 4.41E−05 | −33.3984127 | Hypo-methylated | PCa\|NT DMRs |
| chr5 | 176279044 | 176279202 | 0.00023371 | −30.92640693 | Hypo-methylated | PCa\|NT DMRs |
| chr5 | 176289294 | 176289950 | 4.29E−05 | −25.96031746 | Hypo-methylated | PCa\|NT DMRs |
| chr5 | 176293233 | 176294572 | 0.002950526 | 26.40756303 | Hyper-methylated | PCa\|NT DMRs |
| chr5 | 176303302 | 176304269 | 0.008511532 | 20.16849817 | Hyper-methylated | PCa\|NT DMRs |
| chr5 | 176427199 | 176427904 | 0.005968871 | 35.82380952 | Hyper-methylated | PCa\|NT DMRs |
| chr5 | 176543868 | 176544125 | 8.03E−08 | 25.63528139 | Hyper-methylated | PCa\|NT DMRs |
| chr5 | 176559407 | 176560304 | 1.07E−15 | 30.94115646 | Hyper-methylated | PCa\|NT DMRs |
| chr5 | 176754635 | 176759474 | 1.24E−09 | 21.87467072 | Hyper-methylated | PCa\|NT DMRs |
| chr5 | 176793961 | 176798031 | 8.06E−08 | −33.29120879 | Hypo-methylated | PCa\|NT DMRs |
| chr5 | 176808043 | 176812219 | 0.005968871 | −40.90952381 | Hypo-methylated | PCa\|NT DMRs |
| chr5 | 176827023 | 176827456 | 2.09E−07 | −25.19835796 | Hypo-methylated | PCa\|NT DMRs |
| chr5 | 176857081 | 176861659 | 2.30E−06 | 38.74897959 | Hyper-methylated | PCa\|NT DMRs |
| chr5 | 176906668 | 176906879 | 0.002822916 | −24.72321429 | Hypo-methylated | PCa\|NT DMRs |
| chr5 | 176934735 | 176936883 | 5.15E−10 | 45.48711485 | Hyper-methylated | PCa\|NT DMRs |
| chr5 | 177036836 | 177036938 | 0.008482405 | 27.1047619 | Hyper-methylated | PCa\|NT DMRs |
| chr5 | 177423236 | 177434558 | 0.003829295 | 22.81118012 | Hyper-methylated | PCa\|NT DMRs |
| chr5 | 177541206 | 177543945 | 5.22E−06 | 26.93316913 | Hyper-methylated | PCa\|NT DMRs |
| chr5 | 177665546 | 177669122 | 6.61E−08 | 42.39115646 | Hyper-methylated | PCa\|NT DMRs |
| chr5 | 177669423 | 177669722 | 0.003622801 | −25.66666667 | Hypo-methylated | PCa\|NT DMRs |
| chr5 | 177811306 | 177811400 | 0.005968871 | −30.03333333 | Hypo-methylated | PCa\|NT DMRs |
| chr5 | 178004027 | 178004518 | 1.39E−13 | 33.03504762 | Hyper-methylated | PCa\|NT DMRs |
| chr5 | 178068598 | 178131950 | 4.67E−13 | −29.28674948 | Hypo-methylated | PCa\|NT DMRs |
| chr5 | 178420361 | 178420638 | 5.42E−05 | 30.72380952 | Hyper-methylated | PCa\|NT DMRs |
| chr5 | 178420776 | 178421025 | 0.00179283 | −29.48392857 | Hypo-methylated | PCa\|NT DMRs |
| chr5 | 178426424 | 178446205 | 8.94E−07 | −28.72993197 | Hypo-methylated | PCa\|NT DMRs |
| chr5 | 178451176 | 178451261 | 6.17E−06 | 26.83333333 | Hyper-methylated | PCa\|NT DMRs |
| chr5 | 178478044 | 178482003 | 7.05E−07 | −34.80654762 | Hypo-methylated | PCa\|NT DMRs |
| chr5 | 178548700 | 178548937 | 2.85E−05 | −28.02637363 | Hypo-methylated | PCa\|NT DMRs |
| chr5 | 178553528 | 178557175 | 9.23E−06 | −27.80392157 | Hypo-methylated | PCa\|NT DMRs |
| chr5 | 178565002 | 178566681 | 7.56E−05 | −28.61247166 | Hypo-methylated | PCa\|NT DMRs |
| chr5 | 178580530 | 178585775 | 1.23E−08 | −26.2518315 | Hypo-methylated | PCa\|NT DMRs |
| chr5 | 178594527 | 178598557 | 0.004906295 | −26.25555556 | Hypo-methylated | PCa\|NT DMRs |
| chr5 | 178605205 | 178631811 | 4.32E−12 | −26.33161905 | Hypo-methylated | PCa\|NT DMRs |
| chr5 | 178635377 | 178636919 | 2.31E−05 | −30.95824176 | Hypo-methylated | PCa\|NT DMRs |
| chr5 | 178689164 | 178703505 | 1.10E−13 | −23.37579088 | Hypo-methylated | PCa\|NT DMRs |
| chr5 | 178712514 | 178728014 | 7.83E−16 | −30.36096799 | Hypo-methylated | PCa\|NT DMRs |
| chr5 | 178779650 | 178793764 | 2.04E−09 | −27.87814408 | Hypo-methylated | PCa\|NT DMRs |
| chr5 | 178802025 | 178837641 | 3.29E−10 | −30.3530303 | Hypo-methylated | PCa\|NT DMRs |
| chr5 | 178855579 | 178899951 | 1.94E−17 | −27.63369408 | Hypo-methylated | PCa\|NT DMRs |
| chr5 | 178913310 | 178955544 | 8.69E−12 | −24.85962733 | Hypo-methylated | PCa\|NT DMRs |
| chr5 | 179075628 | 179075889 | 0.005968871 | −50.38857143 | Hypo-methylated | PCa\|NT DMRs |
| chr5 | 179097420 | 179097647 | 0.001969641 | 47.26587302 | Hyper-methylated | PCa\|NT DMRs |
| chr5 | 179193234 | 179200782 | 0.000395643 | 20.49950739 | Hyper-methylated | PCa\|NT DMRs |
| chr5 | 179218651 | 179219675 | 0.001969641 | 37.42301587 | Hyper-methylated | PCa\|NT DMRs |
| chr5 | 179333400 | 179333543 | 1.97E−05 | 26.67142857 | Hyper-methylated | PCa\|NT DMRs |
| chr5 | 179344323 | 179346713 | 0.001969641 | 27.69047619 | Hyper-methylated | PCa\|NT DMRs |
| chr5 | 179527452 | 179528960 | 0.000564835 | −24.71575092 | Hypo-methylated | PCa\|NT DMRs |
| chr5 | 179780971 | 179783246 | 2.99E−08 | 25.31207483 | Hyper-methylated | PCa\|NT DMRs |
| chr5 | 179894247 | 179894412 | 0.000205174 | −44.60714286 | Hypo-methylated | PCa\|NT DMRs |
| chr5 | 180022975 | 180023558 | 1.43E−05 | −44.29579832 | Hypo-methylated | PCa\|NT DMRs |
| chr5 | 180068127 | 180072089 | 4.67E−09 | −20.55042735 | Hypo-methylated | PCa\|NT DMRs |
| chr5 | 180072405 | 180072625 | 6.17E−06 | 31.67619048 | Hyper-methylated | PCa\|NT DMRs |
| chr5 | 180159361 | 180175137 | 0.000377317 | −25.47202381 | Hypo-methylated | PCa\|NT DMRs |
| chr5 | 180485943 | 180486892 | 7.44E−26 | 22.19529347 | Hyper-methylated | PCa\|NT DMRs |
| chr5 | 180591658 | 180591965 | 0.005574525 | 20.93073593 | Hyper-methylated | PCa\|NT DMRs |
| chr6 | 179543 | 179698 | 6.42E−05 | −29.20899471 | Hypo-methylated | PCa\|NT DMRs |
| chr6 | 374510 | 375214 | 0.000974795 | −23.39285714 | Hypo-methylated | PCa\|NT DMRs |
| chr6 | 401453 | 401963 | 4.39E−06 | −26.48148148 | Hypo-methylated | PCa\|NT DMRs |
| chr6 | 448943 | 449244 | 0.000199792 | −21.03809524 | Hypo-methylated | PCa\|NT DMRs |
| chr6 | 586206 | 586271 | 0.005968871 | −47.11428571 | Hypo-methylated | PCa\|NT DMRs |
| chr6 | 1599124 | 1602009 | 8.81E−06 | 37.46616541 | Hyper-methylated | PCa\|NT DMRs |
| chr6 | 1602048 | 1602138 | 0.000913082 | 24.81632653 | Hyper-methylated | PCa\|NT DMRs |
| chr6 | 1614192 | 1615653 | 6.67E−25 | 34.65833333 | Hyper-methylated | PCa\|NT DMRs |
| chr6 | 1619004 | 1621083 | 6.88E−24 | 36.56551587 | Hyper-methylated | PCa\|NT DMRs |
| chr6 | 2478053 | 2478181 | 0.008482405 | −49.23333333 | Hypo-methylated | PCa\|NT DMRs |
| chr6 | 2753283 | 2753533 | 0.005968871 | −35.06666667 | Hypo-methylated | PCa\|NT DMRs |
| chr6 | 2789228 | 2821488 | 1.84E−05 | 21.73351648 | Hyper-methylated | PCa\|NT DMRs |
| chr6 | 2841821 | 2858194 | 1.34E−17 | 40.56516291 | Hyper-methylated | PCa\|NT DMRs |
| chr6 | 3022205 | 3024654 | 2.98E−08 | 39.60564374 | Hyper-methylated | PCa\|NT DMRs |
| chr6 | 3284471 | 3285031 | 0.000847246 | −38.05274725 | Hypo-methylated | PCa\|NT DMRs |
| chr6 | 3438580 | 3438728 | 0.005968871 | −54.2 | Hypo-methylated | PCa\|NT DMRs |
| chr6 | 3762809 | 3762975 | 0.005968871 | −55.6 | Hypo-methylated | PCa\|NT DMRs |
| chr6 | 3840805 | 3840842 | 0.005968871 | −59.72380952 | Hypo-methylated | PCa\|NT DMRs |
| chr6 | 4078805 | 4079528 | 3.69E−05 | 21.59261419 | Hyper-methylated | PCa\|NT DMRs |
| chr6 | 4603561 | 4603735 | 0.00064571 | 24.75170068 | Hyper-methylated | PCa\|NT DMRs |
| chr6 | 5026568 | 5026995 | 0.000189331 | 32.65151515 | Hyper-methylated | PCa\|NT DMRs |
| chr6 | 5116366 | 5116739 | 0.008482405 | −38.85714286 | Hypo-methylated | PCa\|NT DMRs |
| chr6 | 6546375 | 6546943 | 1.52E−16 | 29.32623716 | Hyper-methylated | PCa\|NT DMRs |
| chr6 | 6687243 | 6687653 | 0.002094 | −22.41468254 | Hypo-methylated | PCa\|NT DMRs |
| chr6 | 6695225 | 6695396 | 0.005968871 | 29.82857143 | Hyper-methylated | PCa\|NT DMRs |
| chr6 | 7201216 | 7201456 | 0.00064571 | −40.59183673 | Hypo-methylated | PCa\|NT DMRs |
| chr6 | 7236055 | 7237615 | 1.97E−05 | −49.41428571 | Hypo-methylated | PCa\|NT DMRs |
| chr6 | 9140478 | 9140696 | 0.001969641 | −57.87142857 | Hypo-methylated | PCa\|NT DMRs |
| chr6 | 9971933 | 9976038 | 5.94E−06 | −26.52727273 | Hypo-methylated | PCa\|NT DMRs |
| chr6 | 10113590 | 10113704 | 4.45E−08 | 32.07619048 | Hyper-methylated | PCa\|NT DMRs |
| chr6 | 10381097 | 10382303 | 6.90E−15 | 43.9232684 | Hyper-methylated | PCa\|NT DMRs |
| chr6 | 10385142 | 10399289 | 7.81E−15 | 37.1681606 | Hyper-methylated | PCa\|NT DMRs |
| chr6 | 10405100 | 10410280 | 4.81E−06 | 33.86755952 | Hyper-methylated | PCa\|NT DMRs |
| chr6 | 10415766 | 10419590 | 1.85E−19 | 39.92361647 | Hyper-methylated | PCa\|NT DMRs |
| chr6 | 10691507 | 10691663 | 0.001969641 | −46.83333333 | Hypo-methylated | PCa\|NT DMRs |
| chr6 | 11154457 | 11154594 | 0.005968871 | 20.0047619 | Hyper-methylated | PCa\|NT DMRs |
| chr6 | 12262604 | 12288668 | 0.007959104 | −33.88707483 | Hypo-methylated | PCa\|NT DMRs |
| chr6 | 13015061 | 13015280 | 0.000145366 | −40.30510204 | Hypo-methylated | PCa\|NT DMRs |
| chr6 | 13274132 | 13276250 | 0.001042833 | 45.01571429 | Hyper-methylated | PCa\|NT DMRs |
| chr6 | 13797662 | 13798225 | 6.42E−05 | −51.94179894 | Hypo-methylated | PCa\|NT DMRs |
| chr6 | 14622366 | 14622427 | 0.008482405 | −22.45714286 | Hypo-methylated | PCa\|NT DMRs |
| chr6 | 15074182 | 15074418 | 0.001969641 | −30.47619048 | Hypo-methylated | PCa\|NT DMRs |
| chr6 | 15222335 | 15222736 | 0.003881641 | −52.50529101 | Hypo-methylated | PCa\|NT DMRs |
| chr6 | 15244752 | 15245034 | 5.53E−08 | 38 | Hyper-methylated | PCa\|NT DMRs |
| chr6 | 15501549 | 15502597 | 0.004553481 | 33.15873016 | Hyper-methylated | PCa\|NT DMRs |
| chr6 | 17087054 | 17087115 | 0.005968871 | −33.94761905 | Hypo-methylated | PCa\|NT DMRs |
| chr6 | 17212939 | 17213148 | 0.000205174 | −48.89285714 | Hypo-methylated | PCa\|NT DMRs |
| chr6 | 17702211 | 17702631 | 0.001789867 | 30.17460317 | Hyper-methylated | PCa\|NT DMRs |
| chr6 | 18122400 | 18122551 | 4.25E−07 | 27.74711779 | Hyper-methylated | PCa\|NT DMRs |
| chr6 | 20024141 | 20024570 | 5.84E−05 | 57.53571429 | Hyper-methylated | PCa\|NT DMRs |
| chr6 | 20062204 | 20068841 | 0.003809353 | 27.61190476 | Hyper-methylated | PCa\|NT DMRs |
| chr6 | 20320031 | 20320412 | 0.001969641 | 54.65079365 | Hyper-methylated | PCa\|NT DMRs |
| chr6 | 21479608 | 21479728 | 0.005968871 | −62.82857143 | Hypo-methylated | PCa\|NT DMRs |
| chr6 | 21825744 | 21847664 | 0.001969641 | 52.73412698 | Hyper-methylated | PCa\|NT DMRs |
| chr6 | 25035868 | 25042679 | 0.000205174 | 54.375 | Hyper-methylated | PCa\|NT DMRs |
| chr6 | 26122004 | 26122175 | 6.42E−05 | −30.49206349 | Hypo-methylated | PCa\|NT DMRs |
| chr6 | 26183478 | 26188847 | 3.15E−07 | 29.50612245 | Hyper-methylated | PCa\|NT DMRs |
| chr6 | 27173525 | 27173889 | 0.000111427 | 26.38809524 | Hyper-methylated | PCa\|NT DMRs |
| chr6 | 27205735 | 27206537 | 1.55E−05 | 29.51490683 | Hyper-methylated | PCa\|NT DMRs |
| chr6 | 27279861 | 27280084 | 0.000632596 | 26.83116883 | Hyper-methylated | PCa\|NT DMRs |
| chr6 | 27555535 | 27555849 | 0.005968871 | −32.13619048 | Hypo-methylated | PCa\|NT DMRs |
| chr6 | 27730428 | 27739947 | 0.009924186 | −26.18730159 | Hypo-methylated | PCa\|NT DMRs |
| chr6 | 27798649 | 27798699 | 0.008234951 | 33.76571429 | Hyper-methylated | PCa\|NT DMRs |
| chr6 | 27832041 | 27832231 | 0.005968871 | 27.54285714 | Hyper-methylated | PCa\|NT DMRs |
| chr6 | 27940212 | 27983636 | 0.000771292 | −26.36446886 | Hypo-methylated | PCa\|NT DMRs |
| chr6 | 28131798 | 28143819 | 6.42E−05 | −23.28571429 | Hypo-methylated | PCa\|NT DMRs |
| chr6 | 28740454 | 28744031 | 1.60E−16 | 20.47741935 | Hyper-methylated | PCa\|NT DMRs |
| chr6 | 28833281 | 28833678 | 0.003702731 | −22.88095238 | Hypo-methylated | PCa\|NT DMRs |
| chr6 | 28956466 | 28956666 | 5.94E−06 | 33.17229437 | Hyper-methylated | PCa\|NT DMRs |
| chr6 | 29541910 | 29589632 | 0.000370938 | −22.4047619 | Hypo-methylated | PCa\|NT DMRs |
| chr6 | 29595299 | 29595695 | 1.29E−05 | 24.36521739 | Hyper-methylated | PCa\|NT DMRs |
| chr6 | 29942977 | 29943409 | 6.42E−05 | 43.75661376 | Hyper-methylated | PCa\|NT DMRs |
| chr6 | 30466509 | 30466709 | 0.000205174 | −28.32142857 | Hypo-methylated | PCa\|NT DMRs |
| chr6 | 30649262 | 30653871 | 4.26E−13 | 38.77073171 | Hyper-methylated | PCa\|NT DMRs |
| chr6 | 30710878 | 30712542 | 3.13E−21 | 30.53610519 | Hyper-methylated | PCa\|NT DMRs |
| chr6 | 30852823 | 30854162 | 0.005968871 | −28.15714286 | Hypo-methylated | PCa\|NT DMRs |
| chr6 | 30902675 | 30920836 | 0.009924186 | −22.01587302 | Hypo-methylated | PCa\|NT DMRs |
| chr6 | 31166348 | 31170470 | 0.003809353 | 31.44047619 | Hyper-methylated | PCa\|NT DMRs |
| chr6 | 31276303 | 31276798 | 3.85E−08 | 27.8752381 | Hyper-methylated | PCa\|NT DMRs |
| chr6 | 31525861 | 31528136 | 3.44E−06 | 42.26755952 | Hyper-methylated | PCa\|NT DMRs |
| chr6 | 31539735 | 31540201 | 0.000226774 | 20.65396825 | Hyper-methylated | PCa\|NT DMRs |
| chr6 | 31650760 | 31650877 | 0.000137206 | 21.28730159 | Hyper-methylated | PCa\|NT DMRs |
| chr6 | 31830234 | 31831639 | 2.49E−21 | 26.98251488 | Hyper-methylated | PCa\|NT DMRs |
| chr6 | 31844592 | 31848535 | 0.00023371 | −34.2680776 | Hypo-methylated | PCa\|NT DMRs |
| chr6 | 32847660 | 32847758 | 0.00064571 | 25.87755102 | Hyper-methylated | PCa\|NT DMRs |
| chr6 | 33245438 | 33246044 | 1.44E−05 | 36.3968254 | Hyper-methylated | PCa\|NT DMRs |
| chr6 | 33282713 | 33283190 | 1.68E−07 | −28.75446429 | Hypo-methylated | PCa\|NT DMRs |
| chr6 | 33398866 | 33401761 | 0.001400829 | 29.54761905 | Hyper-methylated | PCa\|NT DMRs |
| chr6 | 33501243 | 33501502 | 0.000396159 | 24.21190476 | Hyper-methylated | PCa\|NT DMRs |
| chr6 | 33519032 | 33519095 | 0.003448944 | −38.57823129 | Hypo-methylated | PCa\|NT DMRs |
| chr6 | 33560929 | 33561176 | 1.42E−05 | 23.45748299 | Hyper-methylated | PCa\|NT DMRs |
| chr6 | 33657868 | 33664376 | 0.000357616 | 25.68903319 | Hyper-methylated | PCa\|NT DMRs |
| chr6 | 33848721 | 33848882 | 0.001969641 | −35.80952381 | Hypo-methylated | PCa\|NT DMRs |
| chr6 | 33927011 | 33943838 | 0.001969641 | 35.24206349 | Hyper-methylated | PCa\|NT DMRs |
| chr6 | 34033937 | 34033991 | 0.001969641 | −53.0952381 | Hypo-methylated | PCa\|NT DMRs |
| chr6 | 34191048 | 34191493 | 0.000328483 | −28.32380952 | Hypo-methylated | PCa\|NT DMRs |
| chr6 | 34634231 | 34640215 | 6.42E−05 | 26.98941799 | Hyper-methylated | PCa\|NT DMRs |
| chr6 | 35108879 | 35109894 | 9.59E−06 | 20.07722008 | Hyper-methylated | PCa\|NT DMRs |
| chr6 | 35145513 | 35181430 | 0.00012477 | 20.32553191 | Hyper-methylated | PCa\|NT DMRs |
| chr6 | 35182352 | 35182816 | 0.00028029 | 21.97587302 | Hyper-methylated | PCa\|NT DMRs |
| chr6 | 35453935 | 35454394 | 1.33E−17 | −50.04069264 | Hypo-methylated | PCa\|NT DMRs |
| chr6 | 35471346 | 35471404 | 6.42E−05 | 50.57142857 | Hyper-methylated | PCa\|NT DMRs |
| chr6 | 35679117 | 35688612 | 0.006225823 | 31.11428571 | Hyper-methylated | PCa\|NT DMRs |
| chr6 | 35698308 | 35698376 | 0.005968871 | 35.2 | Hyper-methylated | PCa\|NT DMRs |
| chr6 | 36703335 | 36703676 | 0.000205174 | −28.96428571 | Hypo-methylated | PCa\|NT DMRs |
| chr6 | 36737479 | 36738133 | 3.39E−05 | 41.62554113 | Hyper-methylated | PCa\|NT DMRs |
| chr6 | 36799121 | 36807496 | 0.00098106 | 24.6752381 | Hyper-methylated | PCa\|NT DMRs |
| chr6 | 36929879 | 36930841 | 2.01E−05 | 29.85595238 | Hyper-methylated | PCa\|NT DMRs |
| chr6 | 37483710 | 37484986 | 0.000117699 | −36.5961039 | Hypo-methylated | PCa\|NT DMRs |
| chr6 | 37750852 | 37751717 | 2.82E−09 | 39.7485348 | Hyper-methylated | PCa\|NT DMRs |
| chr6 | 39194632 | 39196450 | 5.91E−08 | 35.98015873 | Hyper-methylated | PCa\|NT DMRs |
| chr6 | 39964783 | 39990818 | 1.31E−06 | −25.0521164 | Hypo-methylated | PCa\|NT DMRs |
| chr6 | 40114717 | 40249785 | 3.32E−06 | −30.84960317 | Hypo-methylated | PCa\|NT DMRs |
| chr6 | 40846175 | 40880637 | 0.005436464 | −24.50250627 | Hypo-methylated | PCa\|NT DMRs |
| chr6 | 41153817 | 41153975 | 0.000822388 | 24.84285714 | Hyper-methylated | PCa\|NT DMRs |
| chr6 | 41394852 | 41395312 | 0.001580415 | 36.15108225 | Hyper-methylated | PCa\|NT DMRs |
| chr6 | 41437525 | 41438869 | 2.36E−06 | 26.49887218 | Hyper-methylated | PCa\|NT DMRs |
| chr6 | 41460262 | 41471123 | 0.000812507 | 26.69142857 | Hyper-methylated | PCa\|NT DMRs |
| chr6 | 41472290 | 41472432 | 0.007563547 | 23.48268398 | Hyper-methylated | PCa\|NT DMRs |
| chr6 | 41473448 | 41496061 | 0.000219662 | −27.72420635 | Hypo-methylated | PCa\|NT DMRs |
| chr6 | 41528285 | 41529051 | 1.60E−10 | 47.8 | Hyper-methylated | PCa\|NT DMRs |
| chr6 | 41604500 | 41605021 | 1.67E−08 | −25.06515679 | Hypo-methylated | PCa\|NT DMRs |
| chr6 | 41605297 | 41607280 | 1.05E−24 | 29.75810594 | Hyper-methylated | PCa\|NT DMRs |
| chr6 | 41903506 | 41905047 | 0.003448944 | 36.15306122 | Hyper-methylated | PCa\|NT DMRs |
| chr6 | 41975678 | 41978553 | 6.42E−05 | 51.61904762 | Hyper-methylated | PCa\|NT DMRs |
| chr6 | 42015967 | 42016245 | 0.000974795 | 21.32142857 | Hyper-methylated | PCa\|NT DMRs |
| chr6 | 42067286 | 42067648 | 0.000205174 | −44.09583333 | Hypo-methylated | PCa\|NT DMRs |
| chr6 | 42247165 | 42247373 | 0.001969641 | 47.51587302 | Hyper-methylated | PCa\|NT DMRs |
| chr6 | 42387065 | 42397211 | 0.00641359 | 59.08174603 | Hyper-methylated | PCa\|NT DMRs |
| chr6 | 42521978 | 42522140 | 1.27E−06 | 29.84166667 | Hyper-methylated | PCa\|NT DMRs |
| chr6 | 42878755 | 42883814 | 0.001235857 | 20.39466667 | Hyper-methylated | PCa\|NT DMRs |
| chr6 | 43235516 | 43235576 | 0.001969641 | −48.5 | Hypo-methylated | PCa\|NT DMRs |
| chr6 | 43237315 | 43243045 | 1.63E−09 | 24.83333333 | Hyper-methylated | PCa\|NT DMRs |
| chr6 | 43276295 | 43276479 | 0.001969641 | 22.33333333 | Hyper-methylated | PCa\|NT DMRs |
| chr6 | 43612900 | 43613077 | 3.32E−06 | 20.6547619 | Hyper-methylated | PCa\|NT DMRs |
| chr6 | 43670519 | 43670738 | 0.005968871 | 32.22380952 | Hyper-methylated | PCa\|NT DMRs |
| chr6 | 43755465 | 43796596 | 8.73E−05 | 33.14217687 | Hyper-methylated | PCa\|NT DMRs |
| chr6 | 43966771 | 43971921 | 2.52E−07 | 25.18606702 | Hyper-methylated | PCa\|NT DMRs |
| chr6 | 44186649 | 44187596 | 5.52E−05 | 22.53251232 | Hyper-methylated | PCa\|NT DMRs |
| chr6 | 44199352 | 44201403 | 0.00064571 | 45.55102041 | Hyper-methylated | PCa\|NT DMRs |
| chr6 | 44524551 | 44529286 | 2.02E−05 | 31.82804233 | Hyper-methylated | PCa\|NT DMRs |
| chr6 | 44621542 | 44621871 | 0.000205174 | 41.67083333 | Hyper-methylated | PCa\|NT DMRs |
| chr6 | 45390683 | 45399745 | 6.77E−23 | 25.78634921 | Hyper-methylated | PCa\|NT DMRs |
| chr6 | 48696432 | 48842884 | 3.55E−06 | −23.42089947 | Hypo-methylated | PCa\|NT DMRs |
| chr6 | 49047159 | 49270558 | 0.001241245 | −26.66719577 | Hypo-methylated | PCa\|NT DMRs |
| chr6 | 49953415 | 49993384 | 0.000481535 | −33.3994709 | Hypo-methylated | PCa\|NT DMRs |
| chr6 | 50784964 | 50785335 | 5.94E−06 | 41.07792208 | Hyper-methylated | PCa\|NT DMRs |
| chr6 | 50787591 | 50817375 | 3.96E−32 | 26.77379078 | Hyper-methylated | PCa\|NT DMRs |
| chr6 | 52227389 | 52228001 | 6.01E−15 | 20.91661376 | Hyper-methylated | PCa\|NT DMRs |
| chr6 | 52268531 | 52268827 | 0.00064571 | −46.86734694 | Hypo-methylated | PCa\|NT DMRs |
| chr6 | 52408933 | 52409436 | 0.001969641 | −35.46598639 | Hypo-methylated | PCa\|NT DMRs |
| chr6 | 53590475 | 53602679 | 0.002585277 | −31.29761905 | Hypo-methylated | PCa\|NT DMRs |
| chr6 | 54203241 | 54602028 | 2.96E−08 | −37.13066667 | Hypo-methylated | PCa\|NT DMRs |
| chr6 | 54723951 | 54874279 | 1.18E−08 | −36.97777778 | Hypo-methylated | PCa\|NT DMRs |
| chr6 | 54937317 | 55010509 | 0.003871035 | −30.91468254 | Hypo-methylated | PCa\|NT DMRs |
| chr6 | 55104788 | 55104870 | 0.000149331 | −36.87445887 | Hypo-methylated | PCa\|NT DMRs |
| chr6 | 56716301 | 56716682 | 0.000319085 | 38.20119048 | Hyper-methylated | PCa\|NT DMRs |
| chr6 | 61902245 | 62843838 | 3.13E−20 | −29.69627976 | Hypo-methylated | PCa\|NT DMRs |
| chr6 | 63202472 | 63809696 | 5.42E−12 | −29.70171958 | Hypo-methylated | PCa\|NT DMRs |
| chr6 | 63963112 | 64042810 | 3.14E−10 | −27.84801587 | Hypo-methylated | PCa\|NT DMRs |
| chr6 | 69201704 | 69255766 | 2.52E−06 | −34.54705882 | Hypo-methylated | PCa\|NT DMRs |
| chr6 | 69514944 | 69650669 | 8.31E−07 | −31.77804233 | Hypo-methylated | PCa\|NT DMRs |
| chr6 | 71039492 | 71085942 | 0.000354065 | −24.01111111 | Hypo-methylated | PCa\|NT DMRs |
| chr6 | 71815029 | 71874731 | 5.94E−06 | −30.82034632 | Hypo-methylated | PCa\|NT DMRs |
| chr6 | 72299560 | 72498578 | 1.49E−06 | −28.33658009 | Hypo-methylated | PCa\|NT DMRs |
| chr6 | 73330693 | 73331680 | 1.26E−16 | 25.21499118 | Hyper-methylated | PCa\|NT DMRs |
| chr6 | 75911644 | 75912132 | 1.55E−08 | 20.56 | Hyper-methylated | PCa\|NT DMRs |
| chr6 | 76059043 | 76059735 | 6.69E−05 | 23.08904762 | Hyper-methylated | PCa\|NT DMRs |
| chr6 | 81368678 | 81371686 | 4.90E−08 | −25.69718615 | Hypo-methylated | PCa\|NT DMRs |
| chr6 | 81647266 | 82051004 | 8.76E−21 | −30.56014652 | Hypo-methylated | PCa\|NT DMRs |
| chr6 | 84485488 | 84521569 | 0.001969641 | −29.50238095 | Hypo-methylated | PCa\|NT DMRs |
| chr6 | 84954352 | 85426573 | 4.03E−13 | −27.63938492 | Hypo-methylated | PCa\|NT DMRs |
| chr6 | 88450700 | 88450769 | 0.004095673 | −51.38095238 | Hypo-methylated | PCa\|NT DMRs |
| chr6 | 88757426 | 88757469 | 0.00064571 | 22.83333333 | Hyper-methylated | PCa\|NT DMRs |
| chr6 | 88803164 | 88818352 | 0.005513661 | −24.28869048 | Hypo-methylated | PCa\|NT DMRs |
| chr6 | 90116046 | 90120255 | 0.00170819 | 35.18315018 | Hyper-methylated | PCa\|NT DMRs |
| chr6 | 96572120 | 96709167 | 3.15E−07 | −22.00816327 | Hypo-methylated | PCa\|NT DMRs |
| chr6 | 97362727 | 97362869 | 0.005968871 | −28.25714286 | Hypo-methylated | PCa\|NT DMRs |
| chr6 | 97558432 | 97562194 | 0.004236974 | 26.75666667 | Hyper-methylated | PCa\|NT DMRs |
| chr6 | 99699790 | 99703151 | 0.001969641 | −23.9984127 | Hypo-methylated | PCa\|NT DMRs |
| chr6 | 100098671 | 100423544 | 5.84E−05 | −25.63730159 | Hypo-methylated | PCa\|NT DMRs |
| chr6 | 101113322 | 101160982 | 0.007959104 | −34.26530612 | Hypo-methylated | PCa\|NT DMRs |
| chr6 | 104474230 | 104588399 | 0.000118733 | −22.54656085 | Hypo-methylated | PCa\|NT DMRs |
| chr6 | 105525000 | 105534993 | 0.00641359 | −27.90873016 | Hypo-methylated | PCa\|NT DMRs |
| chr6 | 107426718 | 107426941 | 0.00179283 | 30.91785714 | Hyper-methylated | PCa\|NT DMRs |
| chr6 | 107527465 | 107550579 | 0.005968871 | −26.09428571 | Hypo-methylated | PCa\|NT DMRs |
| chr6 | 109267275 | 109274465 | 0.000137206 | −29.66857143 | Hypo-methylated | PCa\|NT DMRs |
| chr6 | 109768430 | 109770784 | 0.005968871 | −20.49659864 | Hypo-methylated | PCa\|NT DMRs |
| chr6 | 112627011 | 112671408 | 0.000406444 | −22.66530612 | Hypo-methylated | PCa\|NT DMRs |
| chr6 | 114181300 | 114181445 | 6.93E−06 | −30.48 | Hypo-methylated | PCa\|NT DMRs |
| chr6 | 115849822 | 116137038 | 0.000549619 | −20.6026455 | Hypo-methylated | PCa\|NT DMRs |
| chr6 | 116783000 | 116783480 | 8.07E−14 | 27.44642857 | Hyper-methylated | PCa\|NT DMRs |
| chr6 | 117807999 | 117869525 | 7.79E−05 | 23.67413793 | Hyper-methylated | PCa\|NT DMRs |
| chr6 | 121069769 | 121069819 | 0.00064571 | −26.33061224 | Hypo-methylated | PCa\|NT DMRs |
| chr6 | 123318725 | 124050907 | 9.39E−12 | −32.97692308 | Hypo-methylated | PCa\|NT DMRs |
| chr6 | 124134391 | 125000397 | 7.40E−16 | −32.84366577 | Hypo-methylated | PCa\|NT DMRs |
| chr6 | 126682514 | 127055771 | 1.76E−10 | −25.01034014 | Hypo-methylated | PCa\|NT DMRs |
| chr6 | 127314560 | 127415333 | 5.37E−05 | −32.63595238 | Hypo-methylated | PCa\|NT DMRs |
| chr6 | 129204125 | 129204541 | 2.73E−06 | 27.78677249 | Hyper-methylated | PCa\|NT DMRs |
| chr6 | 129251213 | 129251341 | 5.36E−09 | 33.04395604 | Hyper-methylated | PCa\|NT DMRs |
| chr6 | 132249752 | 132270549 | 0.001969641 | −29.04365079 | Hypo-methylated | PCa\|NT DMRs |
| chr6 | 132850730 | 132970219 | 3.48E−07 | −38.35095238 | Hypo-methylated | PCa\|NT DMRs |
| chr6 | 133307565 | 133355928 | 0.00064571 | −37.67006803 | Hypo-methylated | PCa\|NT DMRs |
| chr6 | 133375149 | 133559606 | 7.37E−05 | −30.5535014 | Hypo-methylated | PCa\|NT DMRs |
| chr6 | 133601782 | 134012715 | 5.85E−12 | −31.2981982 | Hypo-methylated | PCa\|NT DMRs |
| chr6 | 134436382 | 134436761 | 6.79E−07 | 23.37987013 | Hyper-methylated | PCa\|NT DMRs |
| chr6 | 137241530 | 137242368 | 0.000335989 | 28.41208791 | Hyper-methylated | PCa\|NT DMRs |
| chr6 | 137834594 | 137834650 | 0.000974795 | 34.91071429 | Hyper-methylated | PCa\|NT DMRs |
| chr6 | 138723259 | 138724700 | 0.002298126 | 25.79271709 | Hyper-methylated | PCa\|NT DMRs |
| chr6 | 140018202 | 140018442 | 0.005968871 | 32.37142857 | Hyper-methylated | PCa\|NT DMRs |
| chr6 | 142409729 | 142409878 | 3.09E−06 | 22.65986395 | Hyper-methylated | PCa\|NT DMRs |
| chr6 | 143175102 | 143231911 | 0.002193447 | 23.98008658 | Hyper-methylated | PCa\|NT DMRs |
| chr6 | 143247670 | 143248408 | 1.51E−13 | 21.9982684 | Hyper-methylated | PCa\|NT DMRs |
| chr6 | 144585545 | 144587623 | 0.005968871 | −48.99047619 | Hypo-methylated | PCa\|NT DMRs |
| chr6 | 148639792 | 148639837 | 0.005968871 | −28.71428571 | Hypo-methylated | PCa\|NT DMRs |
| chr6 | 149772376 | 149772825 | 1.42E−09 | 31.42593985 | Hyper-methylated | PCa\|NT DMRs |
| chr6 | 151134596 | 151134787 | 0.00064571 | −39.7877551 | Hypo-methylated | PCa\|NT DMRs |
| chr6 | 151187747 | 151188105 | 6.50E−05 | 28.16770186 | Hyper-methylated | PCa\|NT DMRs |
| chr6 | 151345821 | 151358206 | 8.51E−05 | −42.96825397 | Hypo-methylated | PCa\|NT DMRs |
| chr6 | 151365732 | 151388380 | 0.00020572 | −41.08571429 | Hypo-methylated | PCa\|NT DMRs |
| chr6 | 154375360 | 154532518 | 8.73E−08 | −32.4687619 | Hypo-methylated | PCa\|NT DMRs |
| chr6 | 155649902 | 156380644 | 6.21E−26 | −35.32424242 | Hypo-methylated | PCa\|NT DMRs |
| chr6 | 156407322 | 156472820 | 2.86E−05 | −29.87728938 | Hypo-methylated | PCa\|NT DMRs |
| chr6 | 156651838 | 156660827 | 0.000205174 | −30.66130952 | Hypo-methylated | PCa\|NT DMRs |
| chr6 | 157931773 | 157932149 | 1.76E−06 | 41.6547619 | Hyper-methylated | PCa\|NT DMRs |
| chr6 | 158056754 | 158066619 | 0.005968871 | 20.27619048 | Hyper-methylated | PCa\|NT DMRs |
| chr6 | 158185614 | 158185714 | 0.005968871 | 40.99619048 | Hyper-methylated | PCa\|NT DMRs |
| chr6 | 158245997 | 158274880 | 0.003871035 | −42.28571429 | Hypo-methylated | PCa\|NT DMRs |
| chr6 | 158406555 | 158409199 | 1.76E−06 | −51.29325397 | Hypo-methylated | PCa\|NT DMRs |
| chr6 | 158411822 | 158438202 | 7.30E−05 | −34.49455782 | Hypo-methylated | PCa\|NT DMRs |
| chr6 | 158506919 | 158508118 | 7.04E−07 | 37.25687831 | Hyper-methylated | PCa\|NT DMRs |
| chr6 | 158984337 | 158991389 | 3.25E−05 | 27.95918367 | Hyper-methylated | PCa\|NT DMRs |
| chr6 | 159360182 | 159360918 | 4.45E−08 | 36.63396825 | Hyper-methylated | PCa\|NT DMRs |
| chr6 | 159589597 | 159590220 | 8.46E−05 | 30.54786967 | Hyper-methylated | PCa\|NT DMRs |
| chr6 | 160308058 | 160337843 | 0.000121459 | −21.14891775 | Hypo-methylated | PCa\|NT DMRs |
| chr6 | 163570125 | 163570311 | 2.01E−07 | 29.73642857 | Hyper-methylated | PCa\|NT DMRs |
| chr6 | 163756801 | 163757154 | 9.92E−08 | 26.80272109 | Hyper-methylated | PCa\|NT DMRs |
| chr6 | 163817972 | 163818111 | 0.005968871 | 29.4 | Hyper-methylated | PCa\|NT DMRs |
| chr6 | 164012325 | 164030391 | 5.19E−07 | −21.50309524 | Hypo-methylated | PCa\|NT DMRs |
| chr6 | 164322022 | 164340526 | 6.42E−05 | −27.47830688 | Hypo-methylated | PCa\|NT DMRs |
| chr6 | 164379301 | 164393089 | 7.34E−09 | −25.41318681 | Hypo-methylated | PCa\|NT DMRs |
| chr6 | 164480017 | 164506646 | 5.79E−09 | −31.47066667 | Hypo-methylated | PCa\|NT DMRs |
| chr6 | 164525276 | 164527551 | 0.000974795 | −32.78869048 | Hypo-methylated | PCa\|NT DMRs |
| chr6 | 164587673 | 164638187 | 0.00023371 | −29.73761905 | Hypo-methylated | PCa\|NT DMRs |
| chr6 | 166218123 | 166218821 | 1.23E−10 | 25.0032175 | Hyper-methylated | PCa\|NT DMRs |
| chr6 | 166419013 | 166422172 | 1.86E−09 | 23.04761905 | Hyper-methylated | PCa\|NT DMRs |
| chr6 | 166582369 | 166583180 | 1.23E−12 | 31.18582503 | Hyper-methylated | PCa\|NT DMRs |
| chr6 | 166682087 | 166685722 | 0.003174366 | −27.7610119 | Hypo-methylated | PCa\|NT DMRs |
| chr6 | 166746974 | 166747097 | 0.001583444 | 40.73214286 | Hyper-methylated | PCa\|NT DMRs |
| chr6 | 166853680 | 166855458 | 2.84E−06 | 50.11160714 | Hyper-methylated | PCa\|NT DMRs |
| chr6 | 166901692 | 166901855 | 2.85E−05 | 55.61538462 | Hyper-methylated | PCa\|NT DMRs |
| chr6 | 166976448 | 166976809 | 0.009924186 | 26.13492063 | Hyper-methylated | PCa\|NT DMRs |
| chr6 | 167038855 | 167039292 | 0.001969641 | −53.38095238 | Hypo-methylated | PCa\|NT DMRs |
| chr6 | 167070040 | 167088250 | 0.003003689 | 21.6602381 | Hyper-methylated | PCa\|NT DMRs |
| chr6 | 167171091 | 167171454 | 0.00064571 | 30.60204082 | Hyper-methylated | PCa\|NT DMRs |
| chr6 | 167248198 | 167250731 | 3.20E−05 | −36.74659864 | Hypo-methylated | PCa\|NT DMRs |
| chr6 | 167312336 | 167312423 | 0.00064571 | −29.85306122 | Hypo-methylated | PCa\|NT DMRs |
| chr6 | 167559852 | 167559889 | 0.005968871 | 21.94857143 | Hyper-methylated | PCa\|NT DMRs |
| chr6 | 168106965 | 168113392 | 0.000733783 | 29.87142857 | Hyper-methylated | PCa\|NT DMRs |
| chr6 | 168333306 | 168333467 | 0.005968871 | −29.76666667 | Hypo-methylated | PCa\|NT DMRs |
| chr6 | 168785142 | 168788527 | 0.000123403 | 22.69423559 | Hyper-methylated | PCa\|NT DMRs |
| chr6 | 168789291 | 168789748 | 3.44E−06 | −41.31845238 | Hypo-methylated | PCa\|NT DMRs |
| chr6 | 168843001 | 168843280 | 5.92E−06 | 38.73529412 | Hyper-methylated | PCa\|NT DMRs |
| chr6 | 169048991 | 169049164 | 0.00641359 | −40.80952381 | Hypo-methylated | PCa\|NT DMRs |
| chr6 | 169226418 | 169226880 | 4.48E−06 | −34.74285714 | Hypo-methylated | PCa\|NT DMRs |
| chr6 | 169291050 | 169295971 | 0.006410012 | −30.21626984 | Hypo-methylated | PCa\|NT DMRs |
| chr6 | 169435560 | 169476311 | 3.42E−07 | −24.97414966 | Hypo-methylated | PCa\|NT DMRs |
| chr6 | 169487414 | 169497661 | 3.30E−05 | −22.87936508 | Hypo-methylated | PCa\|NT DMRs |
| chr6 | 169542058 | 169558202 | 0.00027677 | −28.80952381 | Hypo-methylated | PCa\|NT DMRs |
| chr6 | 169569524 | 169569685 | 0.001969641 | −36.62380952 | Hypo-methylated | PCa\|NT DMRs |
| chr6 | 169640238 | 169640606 | 0.000481535 | −44.37037037 | Hypo-methylated | PCa\|NT DMRs |
| chr6 | 169740755 | 169741075 | 0.004326525 | −20.81428571 | Hypo-methylated | PCa\|NT DMRs |
| chr6 | 169778035 | 169780994 | 8.70E−08 | −21.76818182 | Hypo-methylated | PCa\|NT DMRs |
| chr6 | 170338190 | 170338768 | 3.06E−06 | 25.13928571 | Hyper-methylated | PCa\|NT DMRs |
| chr6 | 170403221 | 170403994 | 0.00179283 | 36.93452381 | Hyper-methylated | PCa\|NT DMRs |
| chr6 | 170467172 | 170467502 | 2.86E−05 | 37.10989011 | Hyper-methylated | PCa\|NT DMRs |
| chr6 | 170535578 | 170535928 | 0.000190021 | −42.51212121 | Hypo-methylated | PCa\|NT DMRs |
| chr6 | 170556386 | 170557261 | 0.001974972 | −22.63180952 | Hypo-methylated | PCa\|NT DMRs |
| chr6 | 170573899 | 170575017 | 6.50E−05 | −47.21428571 | Hypo-methylated | PCa\|NT DMRs |
| chr6 | 170578845 | 170581299 | 6.79E−07 | 39.63593074 | Hyper-methylated | PCa\|NT DMRs |
| chr6 | 170581883 | 170586134 | 4.47E−08 | −24.48757764 | Hypo-methylated | PCa\|NT DMRs |
| chr7 | 85568 | 85990 | 0.001985874 | −43.95714286 | Hypo-methylated | PCa\|NT DMRs |
| chr7 | 119273 | 119967 | 0.001305585 | 44.37142857 | Hyper-methylated | PCa\|NT DMRs |
| chr7 | 149899 | 150928 | 0.00369512 | 24.17482993 | Hyper-methylated | PCa\|NT DMRs |
| chr7 | 184901 | 185316 | 4.43E−05 | −22.12051282 | Hypo-methylated | PCa\|NT DMRs |
| chr7 | 329190 | 329782 | 8.21E−08 | 22.08046398 | Hyper-methylated | PCa\|NT DMRs |
| chr7 | 334699 | 334741 | 0.001969641 | −26.44444444 | Hypo-methylated | PCa\|NT DMRs |
| chr7 | 373002 | 373649 | 2.49E−07 | −21.49546485 | Hypo-methylated | PCa\|NT DMRs |
| chr7 | 381545 | 382340 | 3.82E−05 | −42.93641457 | Hypo-methylated | PCa\|NT DMRs |
| chr7 | 391295 | 392268 | 9.27E−05 | 36.38765432 | Hyper-methylated | PCa\|NT DMRs |
| chr7 | 396939 | 400660 | 1.67E−07 | 31.06316527 | Hyper-methylated | PCa\|NT DMRs |
| chr7 | 412912 | 413113 | 0.003764982 | 26.35238095 | Hyper-methylated | PCa\|NT DMRs |
| chr7 | 415787 | 416459 | 0.001867853 | 23.9415873 | Hyper-methylated | PCa\|NT DMRs |
| chr7 | 419885 | 420252 | 0.000117699 | 28.20779221 | Hyper-methylated | PCa\|NT DMRs |
| chr7 | 474810 | 476132 | 4.28E−07 | −36.60194805 | Hypo-methylated | PCa\|NT DMRs |
| chr7 | 481826 | 484931 | 0.005513661 | −21.82108844 | Hypo-methylated | PCa\|NT DMRs |
| chr7 | 560525 | 561356 | 1.80E−10 | 29.41608752 | Hyper-methylated | PCa\|NT DMRs |
| chr7 | 577782 | 578623 | 0.000715461 | 34.45238095 | Hyper-methylated | PCa\|NT DMRs |
| chr7 | 601162 | 601454 | 2.39E−05 | −39.30357143 | Hypo-methylated | PCa\|NT DMRs |
| chr7 | 614047 | 614943 | 0.001083007 | −32.50952381 | Hypo-methylated | PCa\|NT DMRs |
| chr7 | 630473 | 630878 | 3.06E−08 | 37.62215321 | Hyper-methylated | PCa\|NT DMRs |
| chr7 | 630960 | 631133 | 0.005968871 | 33.66507937 | Hyper-methylated | PCa\|NT DMRs |
| chr7 | 632596 | 633218 | 6.10E−05 | 42.30805861 | Hyper-methylated | PCa\|NT DMRs |
| chr7 | 641029 | 645781 | 6.70E−18 | 40.15189873 | Hyper-methylated | PCa\|NT DMRs |
| chr7 | 676117 | 676781 | 0.000339627 | −30.45674603 | Hypo-methylated | PCa\|NT DMRs |
| chr7 | 744822 | 745664 | 0.001969641 | −59.55442177 | Hypo-methylated | PCa\|NT DMRs |
| chr7 | 752307 | 752522 | 1.34E−05 | 22.57142857 | Hyper-methylated | PCa\|NT DMRs |
| chr7 | 771824 | 779037 | 5.56E−05 | −33.00779221 | Hypo-methylated | PCa\|NT DMRs |
| chr7 | 953489 | 954045 | 0.001836065 | −24.38988095 | Hypo-methylated | PCa\|NT DMRs |
| chr7 | 959664 | 960120 | 3.05E−06 | −29.72681704 | Hypo-methylated | PCa\|NT DMRs |
| chr7 | 961836 | 965813 | 3.90E−11 | 28.92721088 | Hyper-methylated | PCa\|NT DMRs |
| chr7 | 968401 | 971926 | 0.003553407 | 20.47994987 | Hyper-methylated | PCa\|NT DMRs |
| chr7 | 997743 | 997911 | 0.001969641 | 52.35102041 | Hyper-methylated | PCa\|NT DMRs |
| chr7 | 1021911 | 1022200 | 0.00023371 | −40.72285714 | Hypo-methylated | PCa\|NT DMRs |
| chr7 | 1027898 | 1028614 | 5.13E−07 | 21.77077176 | Hyper-methylated | PCa\|NT DMRs |
| chr7 | 1051846 | 1052439 | 1.35E−06 | 27.77639752 | Hyper-methylated | PCa\|NT DMRs |
| chr7 | 1095255 | 1096685 | 3.29E−05 | 32.22893773 | Hyper-methylated | PCa\|NT DMRs |
| chr7 | 1100049 | 1105373 | 0.002992764 | 23.22228571 | Hyper-methylated | PCa\|NT DMRs |
| chr7 | 1147387 | 1147964 | 0.005574525 | 34.02121212 | Hyper-methylated | PCa\|NT DMRs |
| chr7 | 1184345 | 1184685 | 0.000169316 | −25.50396825 | Hypo-methylated | PCa\|NT DMRs |
| chr7 | 1217681 | 1220518 | 6.35E−06 | −23.27910053 | Hypo-methylated | PCa\|NT DMRs |
| chr7 | 1270161 | 1270186 | 0.001969641 | 29.38095238 | Hyper-methylated | PCa\|NT DMRs |
| chr7 | 1273389 | 1273439 | 0.001181579 | 21.91836735 | Hyper-methylated | PCa\|NT DMRs |
| chr7 | 1273584 | 1273629 | 0.000205174 | 27.36369048 | Hyper-methylated | PCa\|NT DMRs |
| chr7 | 1301746 | 1303468 | 6.60E−05 | −35.12321429 | Hypo-methylated | PCa\|NT DMRs |
| chr7 | 1314101 | 1314608 | 0.002301155 | −21.63389356 | Hypo-methylated | PCa\|NT DMRs |
| chr7 | 1314718 | 1315879 | 2.67E−12 | −28.42898914 | Hypo-methylated | PCa\|NT DMRs |
| chr7 | 1333280 | 1333725 | 3.20E−05 | −24.82417582 | Hypo-methylated | PCa\|NT DMRs |
| chr7 | 1336292 | 1337384 | 0.000474458 | −24.77582418 | Hypo-methylated | PCa\|NT DMRs |
| chr7 | 1362259 | 1362825 | 0.001431904 | −22.53928571 | Hypo-methylated | PCa\|NT DMRs |
| chr7 | 1409656 | 1415820 | 0.000447319 | −20.1347619 | Hypo-methylated | PCa\|NT DMRs |
| chr7 | 1422820 | 1423916 | 0.000226106 | −29.77086835 | Hypo-methylated | PCa\|NT DMRs |
| chr7 | 1493108 | 1495340 | 3.51E−05 | −32.90357143 | Hypo-methylated | PCa\|NT DMRs |
| chr7 | 1503838 | 1504468 | 7.19E−05 | −48.90714286 | Hypo-methylated | PCa\|NT DMRs |
| chr7 | 1548974 | 1553567 | 3.11E−11 | 31.8637827 | Hyper-methylated | PCa\|NT DMRs |
| chr7 | 1556337 | 1562593 | 0.00013312 | −33.9484127 | Hypo-methylated | PCa\|NT DMRs |
| chr7 | 1563266 | 1569378 | 7.74E−05 | −20.02738095 | Hypo-methylated | PCa\|NT DMRs |
| chr7 | 1594787 | 1595521 | 2.83E−09 | −38.16316527 | Hypo-methylated | PCa\|NT DMRs |
| chr7 | 1707639 | 1708142 | 0.000234237 | 30.49761905 | Hyper-methylated | PCa\|NT DMRs |
| chr7 | 1708710 | 1709430 | 2.74E−15 | 30.91932773 | Hyper-methylated | PCa\|NT DMRs |
| chr7 | 1753263 | 1756033 | 0.000457395 | −41.96666667 | Hypo-methylated | PCa\|NT DMRs |
| chr7 | 1772890 | 1774810 | 3.96E−08 | −33.99923195 | Hypo-methylated | PCa\|NT DMRs |
| chr7 | 1775035 | 1777564 | 0.000205174 | −26.625 | Hypo-methylated | PCa\|NT DMRs |
| chr7 | 1858682 | 1858749 | 0.005968871 | −41.1647619 | Hypo-methylated | PCa\|NT DMRs |
| chr7 | 1883642 | 1883877 | 0.003871035 | 22.5922619 | Hyper-methylated | PCa\|NT DMRs |
| chr7 | 1957050 | 1957383 | 0.000286238 | −21.27692308 | Hypo-methylated | PCa\|NT DMRs |
| chr7 | 1978508 | 1982391 | 1.60E−12 | 36.09398496 | Hyper-methylated | PCa\|NT DMRs |
| chr7 | 2057136 | 2060040 | 0.00011419 | 32.45052265 | Hyper-methylated | PCa\|NT DMRs |
| chr7 | 2106060 | 2106454 | 4.41E−05 | 29.33650794 | Hyper-methylated | PCa\|NT DMRs |
| chr7 | 2113487 | 2113695 | 0.000836132 | −37.87301587 | Hypo-methylated | PCa\|NT DMRs |
| chr7 | 2114270 | 2114928 | 2.99E−09 | 35.52488479 | Hyper-methylated | PCa\|NT DMRs |
| chr7 | 2119341 | 2119557 | 0.00064571 | 44.47278912 | Hyper-methylated | PCa\|NT DMRs |
| chr7 | 2208381 | 2208893 | 0.000456684 | −25.11388889 | Hypo-methylated | PCa\|NT DMRs |
| chr7 | 2285423 | 2286439 | 0.003314163 | 32.63952381 | Hyper-methylated | PCa\|NT DMRs |
| chr7 | 2286667 | 2287104 | 0.002988739 | −29.11831502 | Hypo-methylated | PCa\|NT DMRs |
| chr7 | 2289038 | 2289256 | 6.71E−06 | 43.08035714 | Hyper-methylated | PCa\|NT DMRs |
| chr7 | 2473806 | 2474104 | 0.000205174 | −35.16369048 | Hypo-methylated | PCa\|NT DMRs |
| chr7 | 2511042 | 2511432 | 0.00046742 | 26.66753247 | Hyper-methylated | PCa\|NT DMRs |
| chr7 | 2537532 | 2537948 | 0.00064571 | 41.05782313 | Hyper-methylated | PCa\|NT DMRs |
| chr7 | 2558419 | 2558676 | 2.96E−08 | 35.78829365 | Hyper-methylated | PCa\|NT DMRs |
| chr7 | 2568432 | 2570933 | 8.26E−09 | 39.8092437 | Hyper-methylated | PCa\|NT DMRs |
| chr7 | 2570956 | 2571642 | 2.41E−08 | −53.54099379 | Hypo-methylated | PCa\|NT DMRs |
| chr7 | 2602923 | 2607550 | 0.003032307 | 26.08163265 | Hyper-methylated | PCa\|NT DMRs |
| chr7 | 2646739 | 2646946 | 0.001969641 | 29.61904762 | Hyper-methylated | PCa\|NT DMRs |
| chr7 | 2663043 | 2663337 | 0.003871035 | 31.13095238 | Hyper-methylated | PCa\|NT DMRs |
| chr7 | 2669951 | 2670124 | 0.000974795 | −43.09285714 | Hypo-methylated | PCa\|NT DMRs |
| chr7 | 2677315 | 2677563 | 8.17E−05 | 38.81818182 | Hyper-methylated | PCa\|NT DMRs |
| chr7 | 2681361 | 2683061 | 0.000340264 | 26.02678571 | Hyper-methylated | PCa\|NT DMRs |
| chr7 | 2683140 | 2683469 | 0.008581782 | −21.19444444 | Hypo-methylated | PCa\|NT DMRs |
| chr7 | 2718697 | 2719488 | 5.43E−07 | −48.40827068 | Hypo-methylated | PCa\|NT DMRs |
| chr7 | 2719930 | 2722738 | 0.000634922 | −22.72380952 | Hypo-methylated | PCa\|NT DMRs |
| chr7 | 2738855 | 2738926 | 0.002826041 | 21.16326531 | Hyper-methylated | PCa\|NT DMRs |
| chr7 | 2774474 | 2776358 | 2.96E−06 | 45.09187675 | Hyper-methylated | PCa\|NT DMRs |
| chr7 | 2848597 | 2848762 | 0.000481535 | 29.41587302 | Hyper-methylated | PCa\|NT DMRs |
| chr7 | 2866860 | 2872955 | 0.001344447 | 20.83988095 | Hyper-methylated | PCa\|NT DMRs |
| chr7 | 2899611 | 2900193 | 0.001480644 | −36.78190476 | Hypo-methylated | PCa\|NT DMRs |
| chr7 | 2934456 | 2934513 | 0.005968871 | 50.6 | Hyper-methylated | PCa\|NT DMRs |
| chr7 | 2943499 | 2946005 | 0.002656308 | −29.68170426 | Hypo-methylated | PCa\|NT DMRs |
| chr7 | 2963037 | 2966354 | 8.46E−05 | −25.28195489 | Hypo-methylated | PCa\|NT DMRs |
| chr7 | 2993837 | 2999227 | 0.00179283 | 29.57142857 | Hyper-methylated | PCa\|NT DMRs |
| chr7 | 3018392 | 3020589 | 4.43E−10 | 43.97012987 | Hyper-methylated | PCa\|NT DMRs |
| chr7 | 3033261 | 3033409 | 0.001969641 | −50.71428571 | Hypo-methylated | PCa\|NT DMRs |
| chr7 | 3221067 | 3221409 | 0.00064571 | −65.82993197 | Hypo-methylated | PCa\|NT DMRs |
| chr7 | 3279063 | 3286550 | 0.001451125 | −31.67952381 | Hypo-methylated | PCa\|NT DMRs |
| chr7 | 3622737 | 3622977 | 0.003622801 | −32.55952381 | Hypo-methylated | PCa\|NT DMRs |
| chr7 | 4248708 | 4250972 | 0.001684481 | 22.97619048 | Hyper-methylated | PCa\|NT DMRs |
| chr7 | 4365334 | 4456378 | 8.92E−06 | −24.25204082 | Hypo-methylated | PCa\|NT DMRs |
| chr7 | 4653071 | 4653579 | 0.007560755 | 21.3968254 | Hyper-methylated | PCa\|NT DMRs |
| chr7 | 4663826 | 4675953 | 0.000976859 | −21.2858631 | Hypo-methylated | PCa\|NT DMRs |
| chr7 | 4729083 | 4734698 | 0.002094 | −20.44047619 | Hypo-methylated | PCa\|NT DMRs |
| chr7 | 4848822 | 4849577 | 1.31E−06 | 48.67936508 | Hyper-methylated | PCa\|NT DMRs |
| chr7 | 4863232 | 4863472 | 0.005968871 | 41.79047619 | Hyper-methylated | PCa\|NT DMRs |
| chr7 | 4885496 | 4885578 | 0.000205174 | 41.73214286 | Hyper-methylated | PCa\|NT DMRs |
| chr7 | 4920374 | 4922909 | 9.84E−08 | 22.64993895 | Hyper-methylated | PCa\|NT DMRs |
| chr7 | 4955678 | 4960035 | 0.005513661 | −41.75986395 | Hypo-methylated | PCa\|NT DMRs |
| chr7 | 5111823 | 5112718 | 7.54E−05 | 21.46190476 | Hyper-methylated | PCa\|NT DMRs |
| chr7 | 5184143 | 5184310 | 0.000133627 | 22.9914966 | Hyper-methylated | PCa\|NT DMRs |
| chr7 | 5214077 | 5214408 | 0.009503366 | −20.89285714 | Hypo-methylated | PCa\|NT DMRs |
| chr7 | 5243356 | 5243408 | 1.97E−05 | −31.65238095 | Hypo-methylated | PCa\|NT DMRs |
| chr7 | 5313665 | 5314228 | 0.00064571 | −46.08163265 | Hypo-methylated | PCa\|NT DMRs |
| chr7 | 5436680 | 5452873 | 0.005480955 | 21.23414966 | Hyper-methylated | PCa\|NT DMRs |
| chr7 | 5510402 | 5510923 | 5.60E−06 | −20.54797178 | Hypo-methylated | PCa\|NT DMRs |
| chr7 | 5527669 | 5528127 | 2.74E−05 | 37.46691729 | Hyper-methylated | PCa\|NT DMRs |
| chr7 | 5535253 | 5535937 | 1.38E−07 | −42.00186335 | Hypo-methylated | PCa\|NT DMRs |
| chr7 | 5613487 | 5614661 | 0.007222512 | 20.06135531 | Hyper-methylated | PCa\|NT DMRs |
| chr7 | 5626136 | 5626374 | 0.001181579 | −44.73469388 | Hypo-methylated | PCa\|NT DMRs |
| chr7 | 5734835 | 5741722 | 0.000121459 | 29.80411255 | Hyper-methylated | PCa\|NT DMRs |
| chr7 | 6034963 | 6035755 | 0.001241245 | −39.13227513 | Hypo-methylated | PCa\|NT DMRs |
| chr7 | 6199980 | 6203156 | 0.00179283 | −40.17559524 | Hypo-methylated | PCa\|NT DMRs |
| chr7 | 6435794 | 6437019 | 0.002580892 | 34.08134921 | Hyper-methylated | PCa\|NT DMRs |
| chr7 | 6458922 | 6459091 | 0.005968871 | −50.34857143 | Hypo-methylated | PCa\|NT DMRs |
| chr7 | 6463204 | 6465027 | 1.04E−05 | 31.36645022 | Hyper-methylated | PCa\|NT DMRs |
| chr7 | 6474566 | 6475282 | 0.002697142 | 26.12328042 | Hyper-methylated | PCa\|NT DMRs |
| chr7 | 6600894 | 6611746 | 0.001789867 | −27.57936508 | Hypo-methylated | PCa\|NT DMRs |
| chr7 | 6882006 | 6892166 | 0.00118504 | −29.72142857 | Hypo-methylated | PCa\|NT DMRs |
| chr7 | 6930715 | 6934354 | 0.00064571 | −22.09659864 | Hypo-methylated | PCa\|NT DMRs |
| chr7 | 8456923 | 8463768 | 0.001969641 | −25.02380952 | Hypo-methylated | PCa\|NT DMRs |
| chr7 | 9730523 | 9753072 | 0.00641359 | −25.63809524 | Hypo-methylated | PCa\|NT DMRs |
| chr7 | 11012954 | 11013130 | 0.001146289 | −37.92857143 | Hypo-methylated | PCa\|NT DMRs |
| chr7 | 14411827 | 14735428 | 2.15E−05 | −21.86848073 | Hypo-methylated | PCa\|NT DMRs |
| chr7 | 15626429 | 15688034 | 0.00064571 | −28.39795918 | Hypo-methylated | PCa\|NT DMRs |
| chr7 | 15725474 | 15726527 | 5.01E−08 | 20.97248677 | Hyper-methylated | PCa\|NT DMRs |
| chr7 | 15726835 | 15727139 | 0.001969641 | 21.54365079 | Hyper-methylated | PCa\|NT DMRs |
| chr7 | 16890636 | 16890871 | 0.000974795 | 36.07142857 | Hyper-methylated | PCa\|NT DMRs |
| chr7 | 18001089 | 18054101 | 3.82E−07 | −23.41888889 | Hypo-methylated | PCa\|NT DMRs |
| chr7 | 18143706 | 18387381 | 2.12E−06 | −25.15 | Hypo-methylated | PCa\|NT DMRs |
| chr7 | 18833006 | 18833085 | 0.004095673 | −26.97619048 | Hypo-methylated | PCa\|NT DMRs |
| chr7 | 22590069 | 22590220 | 0.002097193 | 24.91836735 | Hyper-methylated | PCa\|NT DMRs |
| chr7 | 23530109 | 23530453 | 6.93E−06 | 40.55396825 | Hyper-methylated | PCa\|NT DMRs |
| chr7 | 25569063 | 25584298 | 0.000118733 | −31.11322751 | Hypo-methylated | PCa\|NT DMRs |
| chr7 | 25934526 | 25935252 | 0.001501564 | 22.73389356 | Hyper-methylated | PCa\|NT DMRs |
| chr7 | 26192189 | 26216465 | 2.32E−07 | 21.98901099 | Hyper-methylated | PCa\|NT DMRs |
| chr7 | 27127534 | 27135348 | 1.38E−12 | 35.56059113 | Hyper-methylated | PCa\|NT DMRs |
| chr7 | 27141679 | 27143236 | 3.93E−11 | 49.08095238 | Hyper-methylated | PCa\|NT DMRs |
| chr7 | 27169137 | 27170067 | 1.33E−08 | −22.6457672 | Hypo-methylated | PCa\|NT DMRs |
| chr7 | 27191524 | 27195204 | 1.33E−07 | 21.14713065 | Hyper-methylated | PCa\|NT DMRs |
| chr7 | 27214660 | 27215710 | 4.08E−15 | −37.0315735 | Hypo-methylated | PCa\|NT DMRs |
| chr7 | 27242048 | 27265523 | 1.06E−31 | 37.02774943 | Hyper-methylated | PCa\|NT DMRs |
| chr7 | 28887427 | 28887706 | 2.79E−05 | −20.33333333 | Hypo-methylated | PCa\|NT DMRs |
| chr7 | 28995117 | 28995552 | 1.43E−05 | 23.96518883 | Hyper-methylated | PCa\|NT DMRs |
| chr7 | 30028084 | 30028297 | 0.000205174 | 44.95535714 | Hyper-methylated | PCa\|NT DMRs |
| chr7 | 31037912 | 31070475 | 4.57E−06 | −25.77063492 | Hypo-methylated | PCa\|NT DMRs |
| chr7 | 31100405 | 31116789 | 0.000570576 | −24.60695971 | Hypo-methylated | PCa\|NT DMRs |
| chr7 | 31125199 | 31174196 | 0.002225326 | −25.60340136 | Hypo-methylated | PCa\|NT DMRs |
| chr7 | 32306595 | 32335481 | 1.52E−07 | −42.74489796 | Hypo-methylated | PCa\|NT DMRs |
| chr7 | 32347627 | 32437622 | 3.15E−05 | −28.69440994 | Hypo-methylated | PCa\|NT DMRs |
| chr7 | 32482461 | 32518978 | 3.13E−07 | −27.25972222 | Hypo-methylated | PCa\|NT DMRs |
| chr7 | 32981742 | 32982455 | 2.06E−16 | 23.23971088 | Hyper-methylated | PCa\|NT DMRs |
| chr7 | 33871558 | 33871895 | 0.003871035 | −44.37142857 | Hypo-methylated | PCa\|NT DMRs |
| chr7 | 33943681 | 33944041 | 5.04E−06 | 20.76022409 | Hyper-methylated | PCa\|NT DMRs |
| chr7 | 34444720 | 34938659 | 4.62E−14 | −33.13625541 | Hypo-methylated | PCa\|NT DMRs |
| chr7 | 35340373 | 35403073 | 0.00064571 | −37.22993197 | Hypo-methylated | PCa\|NT DMRs |
| chr7 | 36007041 | 36007467 | 2.24E−06 | 30.45303777 | Hyper-methylated | PCa\|NT DMRs |
| chr7 | 36011125 | 36018066 | 4.61E−13 | 32.0223518 | Hyper-methylated | PCa\|NT DMRs |
| chr7 | 36564256 | 36584280 | 0.000481535 | −36.06349206 | Hypo-methylated | PCa\|NT DMRs |
| chr7 | 36687754 | 36838381 | 2.19E−08 | −24.1377551 | Hypo-methylated | PCa\|NT DMRs |
| chr7 | 37038702 | 37129765 | 0.00064571 | −28.95442177 | Hypo-methylated | PCa\|NT DMRs |
| chr7 | 37231246 | 37250947 | 0.003314163 | −25.18809524 | Hypo-methylated | PCa\|NT DMRs |
| chr7 | 37392953 | 37393050 | 0.001969641 | 43.08843537 | Hyper-methylated | PCa\|NT DMRs |
| chr7 | 38351103 | 38370875 | 0.006207271 | 24.76309524 | Hyper-methylated | PCa\|NT DMRs |
| chr7 | 38449115 | 38669647 | 1.22E−07 | −33.12797619 | Hypo-methylated | PCa\|NT DMRs |
| chr7 | 39164189 | 39227160 | 0.001098825 | −29.41876751 | Hypo-methylated | PCa\|NT DMRs |
| chr7 | 39273162 | 39443222 | 1.76E−12 | −32.17777778 | Hypo-methylated | PCa\|NT DMRs |
| chr7 | 39497898 | 39514914 | 6.42E−05 | −46.49100529 | Hypo-methylated | PCa\|NT DMRs |
| chr7 | 39579070 | 39586319 | 0.003391914 | −27.2037037 | Hypo-methylated | PCa\|NT DMRs |
| chr7 | 44315769 | 44315996 | 0.003622801 | 34.96428571 | Hyper-methylated | PCa\|NT DMRs |
| chr7 | 44607827 | 44613004 | 0.003510453 | −20.84897959 | Hypo-methylated | PCa\|NT DMRs |
| chr7 | 45068084 | 45068309 | 0.000205174 | 50.33154762 | Hyper-methylated | PCa\|NT DMRs |
| chr7 | 45229350 | 45233748 | 1.15E−05 | −25.54547619 | Hypo-methylated | PCa\|NT DMRs |
| chr7 | 45300230 | 45429858 | 5.02E−12 | −34.73346561 | Hypo-methylated | PCa\|NT DMRs |
| chr7 | 45438807 | 45553939 | 3.61E−11 | −32.90686275 | Hypo-methylated | PCa\|NT DMRs |
| chr7 | 45597605 | 45606284 | 0.000836132 | −22.44708995 | Hypo-methylated | PCa\|NT DMRs |
| chr7 | 45961239 | 45961563 | 2.18E−06 | 21.72481203 | Hyper-methylated | PCa\|NT DMRs |
| chr7 | 46002378 | 46495473 | 4.55E−10 | −29.56834734 | Hypo-methylated | PCa\|NT DMRs |
| chr7 | 47154443 | 47165537 | 0.00064571 | −37.27891156 | Hypo-methylated | PCa\|NT DMRs |
| chr7 | 47231371 | 47262951 | 0.003464153 | −26.63246753 | Hypo-methylated | PCa\|NT DMRs |
| chr7 | 47367493 | 47368403 | 0.000320955 | −21.10582011 | Hypo-methylated | PCa\|NT DMRs |
| chr7 | 47616221 | 47616717 | 0.000840726 | −26.02910053 | Hypo-methylated | PCa\|NT DMRs |
| chr7 | 47620545 | 47620828 | 0.000260936 | 31.88571429 | Hyper-methylated | PCa\|NT DMRs |
| chr7 | 47621795 | 47622199 | 0.001021229 | 20.5995671 | Hyper-methylated | PCa\|NT DMRs |
| chr7 | 47709393 | 47709604 | 0.000205174 | 52.36428571 | Hyper-methylated | PCa\|NT DMRs |
| chr7 | 47786925 | 47803215 | 0.006589535 | −26.35047619 | Hypo-methylated | PCa\|NT DMRs |
| chr7 | 48031355 | 48031800 | 1.27E−08 | 34.22619048 | Hyper-methylated | PCa\|NT DMRs |
| chr7 | 48047274 | 48049568 | 0.004095673 | −29.31746032 | Hypo-methylated | PCa\|NT DMRs |
| chr7 | 48075936 | 48075985 | 0.001111028 | 21.13227513 | Hyper-methylated | PCa\|NT DMRs |
| chr7 | 48083649 | 48086861 | 9.43E−05 | −26.21758242 | Hypo-methylated | PCa\|NT DMRs |
| chr7 | 48331271 | 48882713 | 8.64E−21 | −32.35232558 | Hypo-methylated | PCa\|NT DMRs |
| chr7 | 49814718 | 49814795 | 0.000116533 | 25.83116883 | Hyper-methylated | PCa\|NT DMRs |
| chr7 | 49843083 | 50016555 | 4.82E−06 | −33.85267857 | Hypo-methylated | PCa\|NT DMRs |
| chr7 | 50198161 | 50198986 | 8.30E−07 | −32.42380952 | Hypo-methylated | PCa\|NT DMRs |
| chr7 | 50221938 | 50226954 | 0.001969641 | −34.31984127 | Hypo-methylated | PCa\|NT DMRs |
| chr7 | 50293957 | 50343250 | 1.27E−05 | −40.95396825 | Hypo-methylated | PCa\|NT DMRs |
| chr7 | 50417791 | 50417858 | 0.001969641 | −29.57936508 | Hypo-methylated | PCa\|NT DMRs |
| chr7 | 50431031 | 50468270 | 1.37E−09 | −39.34850088 | Hypo-methylated | PCa\|NT DMRs |
| chr7 | 52155818 | 52155878 | 0.000205174 | 42.21428571 | Hyper-methylated | PCa\|NT DMRs |
| chr7 | 52467816 | 52528725 | 0.002094 | −27.95992063 | Hypo-methylated | PCa\|NT DMRs |
| chr7 | 53172302 | 53234254 | 6.42E−05 | −30.12698413 | Hypo-methylated | PCa\|NT DMRs |
| chr7 | 53286853 | 53287127 | 5.96E−09 | 22.51465201 | Hyper-methylated | PCa\|NT DMRs |
| chr7 | 53453173 | 53701286 | 6.09E−13 | −27.76734694 | Hypo-methylated | PCa\|NT DMRs |
| chr7 | 53861390 | 54511977 | 2.98E−22 | −32.33258145 | Hypo-methylated | PCa\|NT DMRs |
| chr7 | 54659215 | 54732133 | 0.001982559 | −21.03174603 | Hypo-methylated | PCa\|NT DMRs |
| chr7 | 54765896 | 54798598 | 3.60E−06 | −26.52248677 | Hypo-methylated | PCa\|NT DMRs |
| chr7 | 54901215 | 54901260 | 0.00064571 | −57.92789116 | Hypo-methylated | PCa\|NT DMRs |
| chr7 | 55072375 | 55086430 | 4.51E−07 | 23.54591837 | Hyper-methylated | PCa\|NT DMRs |
| chr7 | 55735259 | 55799334 | 2.28E−06 | −25.11813187 | Hypo-methylated | PCa\|NT DMRs |
| chr7 | 55840928 | 55849758 | 0.005772882 | −24.24444444 | Hypo-methylated | PCa\|NT DMRs |
| chr7 | 55885947 | 55936017 | 7.61E−07 | −28.02683983 | Hypo-methylated | PCa\|NT DMRs |
| chr7 | 56033317 | 56046059 | 2.52E−05 | −37.18431373 | Hypo-methylated | PCa\|NT DMRs |
| chr7 | 56120370 | 56127424 | 0.003622801 | −43.57142857 | Hypo-methylated | PCa\|NT DMRs |
| chr7 | 56147132 | 56147286 | 0.00064571 | −48.5952381 | Hypo-methylated | PCa\|NT DMRs |
| chr7 | 56159429 | 56163715 | 0.004540278 | −26.29285714 | Hypo-methylated | PCa\|NT DMRs |
| chr7 | 56193643 | 56231315 | 0.000392715 | −32.55142857 | Hypo-methylated | PCa\|NT DMRs |
| chr7 | 62532592 | 62573974 | 7.46E−07 | −28.23864469 | Hypo-methylated | PCa\|NT DMRs |
| chr7 | 63438308 | 63459428 | 9.13E−06 | −31.39724311 | Hypo-methylated | PCa\|NT DMRs |
| chr7 | 64019216 | 64020792 | 0.005968871 | −26.79904762 | Hypo-methylated | PCa\|NT DMRs |
| chr7 | 64029966 | 64030939 | 4.83E−10 | 24.6031746 | Hyper-methylated | PCa\|NT DMRs |
| chr7 | 64316961 | 64330130 | 7.34E−05 | −25.2552381 | Hypo-methylated | PCa\|NT DMRs |
| chr7 | 64349528 | 64350017 | 1.84E−14 | 34.58228128 | Hyper-methylated | PCa\|NT DMRs |
| chr7 | 65337399 | 65337460 | 0.003871035 | −22.32142857 | Hypo-methylated | PCa\|NT DMRs |
| chr7 | 65508863 | 65509219 | 2.15E−07 | 29.30204082 | Hyper-methylated | PCa\|NT DMRs |
| chr7 | 65967694 | 65968124 | 6.42E−05 | −24.66402116 | Hypo-methylated | PCa\|NT DMRs |
| chr7 | 65968275 | 65969422 | 0.000575877 | −29.34848485 | Hypo-methylated | PCa\|NT DMRs |
| chr7 | 66829609 | 66829834 | 1.99E−07 | 25.38877551 | Hyper-methylated | PCa\|NT DMRs |
| chr7 | 66888256 | 66888455 | 0.005968871 | −34.03238095 | Hypo-methylated | PCa\|NT DMRs |
| chr7 | 66958369 | 66958662 | 0.00179283 | −40.12857143 | Hypo-methylated | PCa\|NT DMRs |
| chr7 | 68887842 | 68887911 | 0.005968871 | −52.62857143 | Hypo-methylated | PCa\|NT DMRs |
| chr7 | 71256811 | 71267196 | 0.002437742 | −29.96455026 | Hypo-methylated | PCa\|NT DMRs |
| chr7 | 71800532 | 71801133 | 3.56E−11 | 20.56692407 | Hyper-methylated | PCa\|NT DMRs |
| chr7 | 72419050 | 72419443 | 0.00641359 | −23.21587302 | Hypo-methylated | PCa\|NT DMRs |
| chr7 | 72838296 | 72838812 | 1.35E−07 | 30.43061224 | Hyper-methylated | PCa\|NT DMRs |
| chr7 | 73072051 | 73081648 | 0.00399983 | −20.44987469 | Hypo-methylated | PCa\|NT DMRs |
| chr7 | 73083583 | 73085702 | 0.000255063 | 24.15610119 | Hyper-methylated | PCa\|NT DMRs |
| chr7 | 73155879 | 73161189 | 3.00E−06 | −29.92624585 | Hypo-methylated | PCa\|NT DMRs |
| chr7 | 73236823 | 73237244 | 0.000117699 | −56.70995671 | Hypo-methylated | PCa\|NT DMRs |
| chr7 | 73244523 | 73247798 | 1.08E−14 | −55.64989177 | Hypo-methylated | PCa\|NT DMRs |
| chr7 | 73300934 | 73305474 | 0.001039378 | 35.61611722 | Hyper-methylated | PCa\|NT DMRs |
| chr7 | 73305581 | 73305739 | 0.005968871 | −31.57428571 | Hypo-methylated | PCa\|NT DMRs |
| chr7 | 73331072 | 73331269 | 0.005968871 | −49.22857143 | Hypo-methylated | PCa\|NT DMRs |
| chr7 | 73437361 | 73438920 | 4.61E−05 | −32.75555556 | Hypo-methylated | PCa\|NT DMRs |
| chr7 | 73442880 | 73444203 | 0.000822388 | 20.62337662 | Hyper-methylated | PCa\|NT DMRs |
| chr7 | 73457452 | 73457743 | 6.42E−05 | −50.6026455 | Hypo-methylated | PCa\|NT DMRs |
| chr7 | 73476300 | 73476511 | 0.003448944 | −27.2244898 | Hypo-methylated | PCa\|NT DMRs |
| chr7 | 73497616 | 73497790 | 0.002585277 | 20.85714286 | Hyper-methylated | PCa\|NT DMRs |
| chr7 | 73498750 | 73498982 | 5.12E−06 | 25.57482993 | Hyper-methylated | PCa\|NT DMRs |
| chr7 | 73507826 | 73509173 | 6.33E−13 | 45.64367015 | Hyper-methylated | PCa\|NT DMRs |
| chr7 | 73720490 | 73721657 | 2.39E−09 | 33.46781609 | Hyper-methylated | PCa\|NT DMRs |
| chr7 | 73818559 | 73819866 | 0.003159484 | −24.6647619 | Hypo-methylated | PCa\|NT DMRs |
| chr7 | 73889890 | 73890169 | 0.007560755 | −24.57857143 | Hypo-methylated | PCa\|NT DMRs |
| chr7 | 73894831 | 73895072 | 5.19E−07 | 33.61904762 | Hyper-methylated | PCa\|NT DMRs |
| chr7 | 73994502 | 73994568 | 0.005968871 | −28.17142857 | Hypo-methylated | PCa\|NT DMRs |
| chr7 | 74074774 | 74075634 | 1.57E−06 | 39.29161905 | Hyper-methylated | PCa\|NT DMRs |
| chr7 | 74075733 | 74086834 | 0.006225823 | −20.26233766 | Hypo-methylated | PCa\|NT DMRs |
| chr7 | 75045443 | 75046477 | 0.00179283 | −25.88988095 | Hypo-methylated | PCa\|NT DMRs |
| chr7 | 75615224 | 75615978 | 0.000123553 | −28.88235294 | Hypo-methylated | PCa\|NT DMRs |
| chr7 | 75687295 | 75687366 | 0.00064571 | −55.30612245 | Hypo-methylated | PCa\|NT DMRs |
| chr7 | 75779637 | 75779924 | 7.94E−10 | 23.75333333 | Hyper-methylated | PCa\|NT DMRs |
| chr7 | 77354038 | 77354284 | 0.00064571 | 25.54761905 | Hyper-methylated | PCa\|NT DMRs |
| chr7 | 77669600 | 77669964 | 0.001709403 | 31.54464286 | Hyper-methylated | PCa\|NT DMRs |
| chr7 | 78498162 | 79031908 | 1.42E−12 | −34.75889724 | Hypo-methylated | PCa\|NT DMRs |
| chr7 | 79114858 | 79368969 | 3.61E−13 | −35.9025641 | Hypo-methylated | PCa\|NT DMRs |
| chr7 | 81168472 | 81305783 | 1.67E−07 | −24.9890873 | Hypo-methylated | PCa\|NT DMRs |
| chr7 | 81422971 | 81440915 | 6.42E−05 | −30.40952381 | Hypo-methylated | PCa\|NT DMRs |
| chr7 | 83368628 | 83783731 | 1.12E−16 | −24.01720969 | Hypo-methylated | PCa\|NT DMRs |
| chr7 | 83909556 | 83938456 | 0.000208485 | −24.07619048 | Hypo-methylated | PCa\|NT DMRs |
| chr7 | 85221008 | 85395494 | 0.001344447 | −35.79464286 | Hypo-methylated | PCa\|NT DMRs |
| chr7 | 85472847 | 85579521 | 4.96E−06 | −22.02747253 | Hypo-methylated | PCa\|NT DMRs |
| chr7 | 85602117 | 86268957 | 4.45E−15 | −34.39265873 | Hypo-methylated | PCa\|NT DMRs |
| chr7 | 92672831 | 92672988 | 1.97E−05 | 24.98571429 | Hyper-methylated | PCa\|NT DMRs |
| chr7 | 96635908 | 96636917 | 3.22E−10 | 33.74111498 | Hyper-methylated | PCa\|NT DMRs |
| chr7 | 96646327 | 96652464 | 7.52E−12 | 36.78391734 | Hyper-methylated | PCa\|NT DMRs |
| chr7 | 96654782 | 96656815 | 0.00083792 | 28.97029478 | Hyper-methylated | PCa\|NT DMRs |
| chr7 | 97498323 | 97498568 | 0.008482405 | 23.81904762 | Hyper-methylated | PCa\|NT DMRs |
| chr7 | 97663616 | 97663883 | 0.003448944 | −24.47346939 | Hypo-methylated | PCa\|NT DMRs |
| chr7 | 97744982 | 97755838 | 0.002416251 | 28.13333333 | Hyper-methylated | PCa\|NT DMRs |
| chr7 | 97795322 | 97795441 | 0.000481535 | −23.26984127 | Hypo-methylated | PCa\|NT DMRs |
| chr7 | 97839946 | 97840075 | 8.31E−07 | 25.76190476 | Hyper-methylated | PCa\|NT DMRs |
| chr7 | 97840799 | 97847454 | 8.66E−09 | −25.60642857 | Hypo-methylated | PCa\|NT DMRs |
| chr7 | 97852407 | 97854186 | 0.001391421 | −35.4952381 | Hypo-methylated | PCa\|NT DMRs |
| chr7 | 97911704 | 97914995 | 0.008073155 | −22.7491342 | Hypo-methylated | PCa\|NT DMRs |
| chr7 | 97920380 | 97920911 | 3.05E−06 | −23.67032967 | Hypo-methylated | PCa\|NT DMRs |
| chr7 | 97921626 | 97927265 | 0.000158614 | −28.38452381 | Hypo-methylated | PCa\|NT DMRs |
| chr7 | 98245716 | 98245858 | 0.008345196 | 24.75 | Hyper-methylated | PCa\|NT DMRs |
| chr7 | 98258710 | 98287247 | 0.000440265 | −25.64583333 | Hypo-methylated | PCa\|NT DMRs |
| chr7 | 98424445 | 98424689 | 6.42E−05 | −26.85714286 | Hypo-methylated | PCa\|NT DMRs |
| chr7 | 98971627 | 98972198 | 6.00E−08 | 26.71868132 | Hyper-methylated | PCa\|NT DMRs |
| chr7 | 99156074 | 99156869 | 3.97E−23 | 22.7885034 | Hyper-methylated | PCa\|NT DMRs |
| chr7 | 99165661 | 99166035 | 0.00064571 | −28.18367347 | Hypo-methylated | PCa\|NT DMRs |
| chr7 | 99177356 | 99178048 | 1.65E−05 | 28.71170635 | Hyper-methylated | PCa\|NT DMRs |
| chr7 | 99204325 | 99208815 | 0.005968871 | −35.76190476 | Hypo-methylated | PCa\|NT DMRs |
| chr7 | 99589389 | 99596385 | 0.009026269 | 29.07097506 | Hyper-methylated | PCa\|NT DMRs |
| chr7 | 99817556 | 99820023 | 7.40E−11 | 24.78277311 | Hyper-methylated | PCa\|NT DMRs |
| chr7 | 100028261 | 100028979 | 4.33E−05 | 23.4273183 | Hyper-methylated | PCa\|NT DMRs |
| chr7 | 100091312 | 100091715 | 9.97E−15 | 28.99415584 | Hyper-methylated | PCa\|NT DMRs |
| chr7 | 100180073 | 100180129 | 0.001969641 | −41.22619048 | Hypo-methylated | PCa\|NT DMRs |
| chr7 | 100422720 | 100423018 | 0.00064571 | −39.9122449 | Hypo-methylated | PCa\|NT DMRs |
| chr7 | 100426084 | 100427131 | 0.000112123 | 42.11071429 | Hyper-methylated | PCa\|NT DMRs |
| chr7 | 100461849 | 100463658 | 0.002487133 | 29.03809524 | Hyper-methylated | PCa\|NT DMRs |
| chr7 | 100464553 | 100466260 | 1.78E−10 | 33.13746867 | Hyper-methylated | PCa\|NT DMRs |
| chr7 | 100488589 | 100489031 | 1.72E−05 | 34.85136054 | Hyper-methylated | PCa\|NT DMRs |
| chr7 | 100550991 | 100555206 | 0.003464153 | −22.88311688 | Hypo-methylated | PCa\|NT DMRs |
| chr7 | 100659891 | 100660212 | 0.004553481 | 24.66666667 | Hyper-methylated | PCa\|NT DMRs |
| chr7 | 100867268 | 100867468 | 0.005968871 | 22.57142857 | Hyper-methylated | PCa\|NT DMRs |
| chr7 | 100872377 | 100876205 | 1.05E−10 | −46.25660377 | Hypo-methylated | PCa\|NT DMRs |
| chr7 | 100879314 | 100882709 | 6.29E−12 | 32.37727273 | Hyper-methylated | PCa\|NT DMRs |
| chr7 | 100943137 | 100943778 | 6.49E−05 | 20.76428571 | Hyper-methylated | PCa\|NT DMRs |
| chr7 | 100946052 | 100946916 | 8.30E−11 | 33.60720093 | Hyper-methylated | PCa\|NT DMRs |
| chr7 | 101261954 | 101262523 | 6.42E−05 | −31.79259259 | Hypo-methylated | PCa\|NT DMRs |
| chr7 | 101273855 | 101274191 | 0.002097193 | 38.57482993 | Hyper-methylated | PCa\|NT DMRs |
| chr7 | 101280380 | 101288502 | 0.00046209 | −35.28571429 | Hypo-methylated | PCa\|NT DMRs |
| chr7 | 101335369 | 101335742 | 0.001013662 | 31.64795918 | Hyper-methylated | PCa\|NT DMRs |
| chr7 | 101342277 | 101351052 | 8.81E−07 | −37.51183673 | Hypo-methylated | PCa\|NT DMRs |
| chr7 | 101360530 | 101362081 | 2.50E−06 | 35.81989796 | Hyper-methylated | PCa\|NT DMRs |
| chr7 | 101391074 | 101391423 | 0.001471804 | −28.27142857 | Hypo-methylated | PCa\|NT DMRs |
| chr7 | 101543446 | 101544937 | 0.002562908 | −53.30952381 | Hypo-methylated | PCa\|NT DMRs |
| chr7 | 101571203 | 101575580 | 0.008581782 | 28.22619048 | Hyper-methylated | PCa\|NT DMRs |
| chr7 | 101578022 | 101579991 | 6.20E−05 | −25.66857143 | Hypo-methylated | PCa\|NT DMRs |
| chr7 | 101584333 | 101584417 | 0.008482405 | −35.28571429 | Hypo-methylated | PCa\|NT DMRs |
| chr7 | 101958901 | 101962182 | 1.36E−08 | 20.38534226 | Hyper-methylated | PCa\|NT DMRs |
| chr7 | 102066109 | 102066575 | 1.01E−08 | 52.66450216 | Hyper-methylated | PCa\|NT DMRs |
| chr7 | 102081660 | 102085939 | 0.002822916 | 27.01964286 | Hyper-methylated | PCa\|NT DMRs |
| chr7 | 105293408 | 105294359 | 0.005968871 | −35.75714286 | Hypo-methylated | PCa\|NT DMRs |
| chr7 | 105319161 | 105319858 | 3.06E−08 | 32.60559006 | Hyper-methylated | PCa\|NT DMRs |
| chr7 | 105517812 | 105517955 | 0.002826041 | 29.95918367 | Hyper-methylated | PCa\|NT DMRs |
| chr7 | 106673369 | 106682188 | 0.000570576 | 30.44338624 | Hyper-methylated | PCa\|NT DMRs |
| chr7 | 107518437 | 107526769 | 0.00025637 | −24.44166667 | Hypo-methylated | PCa\|NT DMRs |
| chr7 | 107641801 | 107642842 | 1.78E−06 | 26.17142857 | Hyper-methylated | PCa\|NT DMRs |
| chr7 | 108573874 | 109435242 | 5.49E−08 | −29.01035197 | Hypo-methylated | PCa\|NT DMRs |
| chr7 | 113505091 | 113718634 | 8.88E−08 | −20.85102041 | Hypo-methylated | PCa\|NT DMRs |
| chr7 | 117410909 | 117498280 | 5.94E−06 | −31.84415584 | Hypo-methylated | PCa\|NT DMRs |
| chr7 | 117854304 | 117854944 | 0.000233379 | 24.02857143 | Hyper-methylated | PCa\|NT DMRs |
| chr7 | 118600016 | 118725787 | 6.99E−05 | −26.67326007 | Hypo-methylated | PCa\|NT DMRs |
| chr7 | 118953370 | 119591869 | 2.07E−13 | −27.33095238 | Hypo-methylated | PCa\|NT DMRs |
| chr7 | 119623727 | 119900893 | 1.30E−09 | −33.15190476 | Hypo-methylated | PCa\|NT DMRs |
| chr7 | 124765830 | 124980314 | 1.14E−06 | −23.40385488 | Hypo-methylated | PCa\|NT DMRs |
| chr7 | 127912088 | 127912382 | 2.13E−06 | 28.63428571 | Hyper-methylated | PCa\|NT DMRs |
| chr7 | 128046447 | 128046586 | 0.001083007 | −24.2952381 | Hypo-methylated | PCa\|NT DMRs |
| chr7 | 128048705 | 128049001 | 0.000205174 | 41.39880952 | Hyper-methylated | PCa\|NT DMRs |
| chr7 | 128430418 | 128431143 | 9.04E−05 | 24.03753501 | Hyper-methylated | PCa\|NT DMRs |
| chr7 | 128470351 | 128474036 | 1.56E−18 | 28.43789683 | Hyper-methylated | PCa\|NT DMRs |
| chr7 | 128509912 | 128510319 | 2.88E−05 | 25.61759834 | Hyper-methylated | PCa\|NT DMRs |
| chr7 | 128519584 | 128520936 | 6.22E−05 | −42.14863946 | Hypo-methylated | PCa\|NT DMRs |
| chr7 | 128573479 | 128573579 | 0.000205174 | 47.86904762 | Hyper-methylated | PCa\|NT DMRs |
| chr7 | 128578726 | 128581589 | 2.07E−05 | 38.11721612 | Hyper-methylated | PCa\|NT DMRs |
| chr7 | 128809044 | 128809288 | 7.00E−10 | 35.59831029 | Hyper-methylated | PCa\|NT DMRs |
| chr7 | 128851432 | 128852053 | 0.009106413 | 24.16269841 | Hyper-methylated | PCa\|NT DMRs |
| chr7 | 129780773 | 129781240 | 0.001731883 | 26.65404762 | Hyper-methylated | PCa\|NT DMRs |
| chr7 | 129794322 | 129794625 | 0.001419801 | −39.43095238 | Hypo-methylated | PCa\|NT DMRs |
| chr7 | 129917732 | 129945716 | 9.63E−06 | −28.29795918 | Hypo-methylated | PCa\|NT DMRs |
| chr7 | 129986609 | 129990875 | 0.001789867 | −23.76455026 | Hypo-methylated | PCa\|NT DMRs |
| chr7 | 130010730 | 130019806 | 2.49E−05 | −31.99447005 | Hypo-methylated | PCa\|NT DMRs |
| chr7 | 130127778 | 130132823 | 1.59E−27 | 21.96573661 | Hyper-methylated | PCa\|NT DMRs |
| chr7 | 130369743 | 130392873 | 0.002215138 | −22.97472527 | Hypo-methylated | PCa\|NT DMRs |
| chr7 | 130432349 | 130490560 | 0.00064571 | −27.92857143 | Hypo-methylated | PCa\|NT DMRs |
| chr7 | 130586419 | 130589355 | 0.004553481 | −26.67037037 | Hypo-methylated | PCa\|NT DMRs |
| chr7 | 131192644 | 131192914 | 0.001888566 | −21.14232804 | Hypo-methylated | PCa\|NT DMRs |
| chr7 | 131242618 | 131243006 | 1.69E−06 | 30.16946779 | Hyper-methylated | PCa\|NT DMRs |
| chr7 | 131308045 | 131308107 | 0.003622801 | 24.32936508 | Hyper-methylated | PCa\|NT DMRs |
| chr7 | 131350332 | 131373284 | 7.57E−05 | −32.67857143 | Hypo-methylated | PCa\|NT DMRs |
| chr7 | 131514718 | 131515085 | 8.49E−05 | 21.51555556 | Hyper-methylated | PCa\|NT DMRs |
| chr7 | 132160221 | 132160827 | 0.000243521 | 27.2961039 | Hyper-methylated | PCa\|NT DMRs |
| chr7 | 134236689 | 134237201 | 0.003448944 | −20.65170068 | Hypo-methylated | PCa\|NT DMRs |
| chr7 | 134851193 | 134851592 | 0.00064571 | 42.98367347 | Hyper-methylated | PCa\|NT DMRs |
| chr7 | 137531886 | 137532401 | 7.66E−13 | 25.55493612 | Hyper-methylated | PCa\|NT DMRs |
| chr7 | 137668416 | 137668618 | 0.008482405 | −49.16666667 | Hypo-methylated | PCa\|NT DMRs |
| chr7 | 138407351 | 138424610 | 0.002097193 | −20.37891156 | Hypo-methylated | PCa\|NT DMRs |
| chr7 | 138761042 | 138761120 | 0.004095673 | −45.57142857 | Hypo-methylated | PCa\|NT DMRs |
| chr7 | 138773705 | 138789703 | 0.006668079 | 22.41190476 | Hyper-methylated | PCa\|NT DMRs |
| chr7 | 140135272 | 140135575 | 1.18E−08 | 33.35515873 | Hyper-methylated | PCa\|NT DMRs |
| chr7 | 140212417 | 140219841 | 0.00095173 | −23.04027778 | Hypo-methylated | PCa\|NT DMRs |
| chr7 | 141589733 | 141720786 | 0.000145366 | −25.14965986 | Hypo-methylated | PCa\|NT DMRs |
| chr7 | 141849501 | 142012611 | 6.93E−06 | −30.46209524 | Hypo-methylated | PCa\|NT DMRs |
| chr7 | 142237672 | 142332431 | 1.52E−07 | −27.58163265 | Hypo-methylated | PCa\|NT DMRs |
| chr7 | 143042512 | 143042867 | 1.06E−06 | 24.44285714 | Hyper-methylated | PCa\|NT DMRs |
| chr7 | 143079114 | 143079495 | 2.38E−07 | 31.69480519 | Hyper-methylated | PCa\|NT DMRs |
| chr7 | 143110984 | 143111353 | 0.005513661 | 21.2755102 | Hyper-methylated | PCa\|NT DMRs |
| chr7 | 144565758 | 144713051 | 0.001969641 | −30.5 | Hypo-methylated | PCa\|NT DMRs |
| chr7 | 145481156 | 145481768 | 0.003622801 | −31.31746032 | Hypo-methylated | PCa\|NT DMRs |
| chr7 | 148198549 | 148308634 | 0.00338426 | −24.97104762 | Hypo-methylated | PCa\|NT DMRs |
| chr7 | 149095044 | 149095100 | 0.009106413 | 26.02380952 | Hyper-methylated | PCa\|NT DMRs |
| chr7 | 149411257 | 149411705 | 0.001182824 | 24.26632653 | Hyper-methylated | PCa\|NT DMRs |
| chr7 | 149412141 | 149412486 | 1.72E−07 | 24.44444444 | Hyper-methylated | PCa\|NT DMRs |
| chr7 | 149566838 | 149567085 | 0.005968871 | 57.24285714 | Hyper-methylated | PCa\|NT DMRs |
| chr7 | 149916642 | 149917350 | 0.000159126 | 25.69607843 | Hyper-methylated | PCa\|NT DMRs |
| chr7 | 150069281 | 150069946 | 7.82E−16 | −31.7047619 | Hypo-methylated | PCa\|NT DMRs |
| chr7 | 150118633 | 150140064 | 0.005030552 | −26.05208333 | Hypo-methylated | PCa\|NT DMRs |
| chr7 | 150328663 | 150329487 | 0.000836132 | 54.07407407 | Hyper-methylated | PCa\|NT DMRs |
| chr7 | 150692222 | 150695535 | 3.23E−07 | 34.98761905 | Hyper-methylated | PCa\|NT DMRs |
| chr7 | 150773386 | 150774524 | 0.004149771 | −24.3010989 | Hypo-methylated | PCa\|NT DMRs |
| chr7 | 150890349 | 150890467 | 0.000205174 | −30.5922619 | Hypo-methylated | PCa\|NT DMRs |
| chr7 | 150951963 | 150954795 | 1.08E−07 | −31.85514593 | Hypo-methylated | PCa\|NT DMRs |
| chr7 | 151077969 | 151078099 | 0.003622801 | 37.41428571 | Hyper-methylated | PCa\|NT DMRs |
| chr7 | 151437844 | 151442405 | 0.008083098 | 28.18507937 | Hyper-methylated | PCa\|NT DMRs |
| chr7 | 151516235 | 151517272 | 0.005513661 | −45.47482993 | Hypo-methylated | PCa\|NT DMRs |
| chr7 | 151535841 | 151536655 | 3.15E−06 | 57.75502646 | Hyper-methylated | PCa\|NT DMRs |
| chr7 | 152210265 | 152210334 | 0.000205174 | 48.89285714 | Hyper-methylated | PCa\|NT DMRs |
| chr7 | 152559079 | 152559308 | 0.00064571 | 30.20068027 | Hyper-methylated | PCa\|NT DMRs |
| chr7 | 152591316 | 152591405 | 0.000205174 | −36.17261905 | Hypo-methylated | PCa\|NT DMRs |
| chr7 | 154705890 | 154706040 | 0.000210841 | 34.6 | Hyper-methylated | PCa\|NT DMRs |
| chr7 | 154813067 | 154859345 | 3.95E−06 | −27.51047619 | Hypo-methylated | PCa\|NT DMRs |
| chr7 | 154863874 | 154879336 | 0.00064571 | −27.77210884 | Hypo-methylated | PCa\|NT DMRs |
| chr7 | 154899588 | 154902631 | 9.81E−07 | −34.54634921 | Hypo-methylated | PCa\|NT DMRs |
| chr7 | 154923361 | 154958943 | 8.28E−07 | −30.12408964 | Hypo-methylated | PCa\|NT DMRs |
| chr7 | 154961253 | 154981577 | 0.00068166 | −25.80416667 | Hypo-methylated | PCa\|NT DMRs |
| chr7 | 154986754 | 154991510 | 1.67E−06 | −26.50125313 | Hypo-methylated | PCa\|NT DMRs |
| chr7 | 154993934 | 155006511 | 0.000149911 | −20.96439909 | Hypo-methylated | PCa\|NT DMRs |
| chr7 | 155039686 | 155049673 | 1.02E−05 | −29.40642857 | Hypo-methylated | PCa\|NT DMRs |
| chr7 | 155058099 | 155080113 | 2.57E−12 | −24.35019305 | Hypo-methylated | PCa\|NT DMRs |
| chr7 | 155307843 | 155318299 | 1.67E−05 | −20.23333333 | Hypo-methylated | PCa\|NT DMRs |
| chr7 | 155386238 | 155386991 | 0.009924186 | 25.23809524 | Hyper-methylated | PCa\|NT DMRs |
| chr7 | 155556567 | 155556757 | 1.69E−06 | 52.38655462 | Hyper-methylated | PCa\|NT DMRs |
| chr7 | 155579930 | 155580536 | 1.21E−17 | 21.74123989 | Hyper-methylated | PCa\|NT DMRs |
| chr7 | 155598430 | 155599414 | 1.98E−09 | 33.88637874 | Hyper-methylated | PCa\|NT DMRs |
| chr7 | 155606684 | 155607230 | 0.000170278 | 34.01984127 | Hyper-methylated | PCa\|NT DMRs |
| chr7 | 155636891 | 155637294 | 0.000783791 | −34.4 | Hypo-methylated | PCa\|NT DMRs |
| chr7 | 156255240 | 156255292 | 0.001969641 | −40.71428571 | Hypo-methylated | PCa\|NT DMRs |
| chr7 | 156696702 | 156700085 | 0.007959104 | −34.6122449 | Hypo-methylated | PCa\|NT DMRs |
| chr7 | 156716094 | 156722985 | 0.009924186 | 24.65555556 | Hyper-methylated | PCa\|NT DMRs |
| chr7 | 156735267 | 156735665 | 1.54E−06 | 24.73854875 | Hyper-methylated | PCa\|NT DMRs |
| chr7 | 156786078 | 156786279 | 0.000783791 | 22.62857143 | Hyper-methylated | PCa\|NT DMRs |
| chr7 | 156800870 | 156802228 | 1.42E−05 | 25.81031746 | Hyper-methylated | PCa\|NT DMRs |
| chr7 | 156805387 | 156810756 | 1.64E−08 | 24.71848739 | Hyper-methylated | PCa\|NT DMRs |
| chr7 | 156814372 | 156826180 | 1.15E−06 | 21.26862027 | Hyper-methylated | PCa\|NT DMRs |
| chr7 | 156917419 | 156917926 | 1.29E−05 | −32.13184524 | Hypo-methylated | PCa\|NT DMRs |
| chr7 | 157078970 | 157082281 | 0.009924186 | −34.69312169 | Hypo-methylated | PCa\|NT DMRs |
| chr7 | 157090015 | 157093261 | 0.000516919 | 25.48263305 | Hyper-methylated | PCa\|NT DMRs |
| chr7 | 157261528 | 157263081 | 2.02E−10 | −23.44690983 | Hypo-methylated | PCa\|NT DMRs |
| chr7 | 157288249 | 157291070 | 2.02E−05 | −25.55132275 | Hypo-methylated | PCa\|NT DMRs |
| chr7 | 157306855 | 157307051 | 0.000974795 | −33.4702381 | Hypo-methylated | PCa\|NT DMRs |
| chr7 | 157315700 | 157320784 | 1.57E−06 | −23.75565476 | Hypo-methylated | PCa\|NT DMRs |
| chr7 | 157378077 | 157378560 | 2.32E−06 | 39.78711485 | Hyper-methylated | PCa\|NT DMRs |
| chr7 | 157405582 | 157406762 | 6.77E−16 | 33.40800866 | Hyper-methylated | PCa\|NT DMRs |
| chr7 | 157461082 | 157461525 | 0.000969909 | 36.485 | Hyper-methylated | PCa\|NT DMRs |
| chr7 | 157648636 | 157648754 | 0.003391914 | 29.50846561 | Hyper-methylated | PCa\|NT DMRs |
| chr7 | 157668107 | 157668187 | 0.005968871 | 40.02857143 | Hyper-methylated | PCa\|NT DMRs |
| chr7 | 157793319 | 157795183 | 2.98E−07 | −37.55824176 | Hypo-methylated | PCa\|NT DMRs |
| chr7 | 157820956 | 157821231 | 0.003448944 | −28.91836735 | Hypo-methylated | PCa\|NT DMRs |
| chr7 | 158030503 | 158030841 | 4.50E−09 | −32.18897243 | Hypo-methylated | PCa\|NT DMRs |
| chr7 | 158110582 | 158111273 | 1.98E−08 | 36.19177489 | Hyper-methylated | PCa\|NT DMRs |
| chr7 | 158201160 | 158201414 | 0.001969641 | 53.41666667 | Hyper-methylated | PCa\|NT DMRs |
| chr7 | 158221366 | 158221418 | 0.001969641 | 48.57142857 | Hyper-methylated | PCa\|NT DMRs |
| chr8 | 157149 | 157227 | 0.003622801 | −23.09126984 | Hypo-methylated | PCa\|NT DMRs |
| chr8 | 335341 | 335523 | 0.000396159 | −42.38571429 | Hypo-methylated | PCa\|NT DMRs |
| chr8 | 495872 | 496327 | 0.008518096 | 22.46825397 | Hyper-methylated | PCa\|NT DMRs |
| chr8 | 523093 | 523250 | 0.00064571 | −58.76326531 | Hypo-methylated | PCa\|NT DMRs |
| chr8 | 635022 | 637739 | 4.47E−08 | −34.59032738 | Hypo-methylated | PCa\|NT DMRs |
| chr8 | 644216 | 644850 | 0.000512756 | −49.7952381 | Hypo-methylated | PCa\|NT DMRs |
| chr8 | 672007 | 675055 | 0.002285154 | 21.10714286 | Hyper-methylated | PCa\|NT DMRs |
| chr8 | 748423 | 754477 | 0.003093471 | −33.20793651 | Hypo-methylated | PCa\|NT DMRs |
| chr8 | 898192 | 898681 | 5.85E−07 | 51.89501134 | Hyper-methylated | PCa\|NT DMRs |
| chr8 | 899474 | 900191 | 6.58E−08 | 38.90828157 | Hyper-methylated | PCa\|NT DMRs |
| chr8 | 1308896 | 1311552 | 0.002950526 | 22.93557423 | Hyper-methylated | PCa\|NT DMRs |
| chr8 | 1365050 | 1365223 | 1.76E−06 | 28.08373016 | Hyper-methylated | PCa\|NT DMRs |
| chr8 | 1728651 | 1729449 | 4.38E−05 | −25.62559524 | Hypo-methylated | PCa\|NT DMRs |
| chr8 | 1803030 | 1805181 | 9.49E−05 | 23.93479853 | Hyper-methylated | PCa\|NT DMRs |
| chr8 | 1807321 | 1807799 | 0.002097193 | −47.42176871 | Hypo-methylated | PCa\|NT DMRs |
| chr8 | 1810232 | 1810429 | 3.66E−05 | −30.8 | Hypo-methylated | PCa\|NT DMRs |
| chr8 | 1894575 | 1896172 | 0.000149336 | −38.43277311 | Hypo-methylated | PCa\|NT DMRs |
| chr8 | 1950305 | 1954120 | 2.77E−14 | 30.27138856 | Hyper-methylated | PCa\|NT DMRs |
| chr8 | 1969030 | 1971998 | 0.00013578 | 20.95145503 | Hyper-methylated | PCa\|NT DMRs |
| chr8 | 2039649 | 2040225 | 1.16E−05 | −24.63613445 | Hypo-methylated | PCa\|NT DMRs |
| chr8 | 2048742 | 2049292 | 8.17E−05 | −20.66233766 | Hypo-methylated | PCa\|NT DMRs |
| chr8 | 2191769 | 2210544 | 1.30E−09 | −33.79047619 | Hypo-methylated | PCa\|NT DMRs |
| chr8 | 2222761 | 2243000 | 2.12E−05 | −29.02571429 | Hypo-methylated | PCa\|NT DMRs |
| chr8 | 2269060 | 2403929 | 2.10E−14 | −37.02970522 | Hypo-methylated | PCa\|NT DMRs |
| chr8 | 2607430 | 2609669 | 0.003093471 | −26.00952381 | Hypo-methylated | PCa\|NT DMRs |
| chr8 | 6631261 | 6633060 | 0.001254235 | −40.56153846 | Hypo-methylated | PCa\|NT DMRs |
| chr8 | 6758409 | 6761867 | 0.007959104 | −37.78231293 | Hypo-methylated | PCa\|NT DMRs |
| chr8 | 7340396 | 7641383 | 0.003223657 | −20.95686275 | Hypo-methylated | PCa\|NT DMRs |
| chr8 | 8560333 | 8560812 | 0.000456684 | 30.17896825 | Hyper-methylated | PCa\|NT DMRs |
| chr8 | 9107225 | 9136077 | 1.37E−06 | −25.60975057 | Hypo-methylated | PCa\|NT DMRs |
| chr8 | 9194614 | 9222629 | 0.008581782 | −20.54880952 | Hypo-methylated | PCa\|NT DMRs |
| chr8 | 9705662 | 9754896 | 6.50E−07 | −26.08178054 | Hypo-methylated | PCa\|NT DMRs |
| chr8 | 9762687 | 9762892 | 7.61E−07 | 20.81168831 | Hyper-methylated | PCa\|NT DMRs |
| chr8 | 9764450 | 9764994 | 1.97E−05 | 21.13311688 | Hyper-methylated | PCa\|NT DMRs |
| chr8 | 9770284 | 9855269 | 3.62E−07 | −22.70229592 | Hypo-methylated | PCa\|NT DMRs |
| chr8 | 10282184 | 10282416 | 0.001969641 | −35.87142857 | Hypo-methylated | PCa\|NT DMRs |
| chr8 | 10393418 | 10393469 | 0.005513661 | −25.56428571 | Hypo-methylated | PCa\|NT DMRs |
| chr8 | 10404809 | 10405127 | 0.001969641 | 31.83260073 | Hyper-methylated | PCa\|NT DMRs |
| chr8 | 10448867 | 10450166 | 0.00068166 | 30.43154762 | Hyper-methylated | PCa\|NT DMRs |
| chr8 | 10568487 | 10568843 | 0.000377317 | 41.29761905 | Hyper-methylated | PCa\|NT DMRs |
| chr8 | 10573606 | 10574501 | 9.51E−10 | 33.43645833 | Hyper-methylated | PCa\|NT DMRs |
| chr8 | 10911919 | 10913565 | 0.004266047 | 20.51948052 | Hyper-methylated | PCa\|NT DMRs |
| chr8 | 11323538 | 11323621 | 0.00023371 | −20.58333333 | Hypo-methylated | PCa\|NT DMRs |
| chr8 | 11470739 | 11471216 | 2.91E−07 | 27.74627976 | Hyper-methylated | PCa\|NT DMRs |
| chr8 | 11508100 | 11508733 | 0.000260936 | −45.73714286 | Hypo-methylated | PCa\|NT DMRs |
| chr8 | 11653401 | 11653492 | 0.000205174 | −45.51904762 | Hypo-methylated | PCa\|NT DMRs |
| chr8 | 11710911 | 11711186 | 0.003871035 | −38.53571429 | Hypo-methylated | PCa\|NT DMRs |
| chr8 | 11735071 | 11735436 | 0.001969641 | 48.00396825 | Hyper-methylated | PCa\|NT DMRs |
| chr8 | 11743803 | 11743915 | 0.001969641 | 41.32380952 | Hyper-methylated | PCa\|NT DMRs |
| chr8 | 11762895 | 11763898 | 0.003448944 | −24.71088435 | Hypo-methylated | PCa\|NT DMRs |
| chr8 | 14424399 | 14532548 | 3.31E−10 | −35.33395445 | Hypo-methylated | PCa\|NT DMRs |
| chr8 | 14640664 | 15042204 | 1.31E−11 | −33.06517857 | Hypo-methylated | PCa\|NT DMRs |
| chr8 | 15125433 | 15231562 | 2.01E−07 | −37.7047619 | Hypo-methylated | PCa\|NT DMRs |
| chr8 | 15263553 | 15263737 | 0.005968871 | −44.94285714 | Hypo-methylated | PCa\|NT DMRs |
| chr8 | 15782085 | 15903541 | 8.96E−05 | −26.41120448 | Hypo-methylated | PCa\|NT DMRs |
| chr8 | 16669253 | 16687516 | 0.000149331 | −32.20562771 | Hypo-methylated | PCa\|NT DMRs |
| chr8 | 17105792 | 17106080 | 0.007425114 | −26.64285714 | Hypo-methylated | PCa\|NT DMRs |
| chr8 | 17270930 | 17271329 | 6.81E−12 | 21.39880952 | Hyper-methylated | PCa\|NT DMRs |
| chr8 | 17533391 | 17533670 | 0.005968871 | −23.14666667 | Hypo-methylated | PCa\|NT DMRs |
| chr8 | 17954994 | 17955260 | 0.000974795 | −55.31845238 | Hypo-methylated | PCa\|NT DMRs |
| chr8 | 18243948 | 18244094 | 0.001969641 | −23.04081633 | Hypo-methylated | PCa\|NT DMRs |
| chr8 | 18244312 | 18257704 | 2.55E−07 | 27.13801843 | Hyper-methylated | PCa\|NT DMRs |
| chr8 | 18871661 | 18872113 | 2.68E−11 | 30.63835264 | Hyper-methylated | PCa\|NT DMRs |
| chr8 | 20160002 | 20160842 | 0.000226106 | 28.1959707 | Hyper-methylated | PCa\|NT DMRs |
| chr8 | 20303498 | 20305063 | 6.42E−05 | −24.68042328 | Hypo-methylated | PCa\|NT DMRs |
| chr8 | 20952800 | 20953016 | 0.005968871 | −51.80952381 | Hypo-methylated | PCa\|NT DMRs |
| chr8 | 21067590 | 21067765 | 0.000205174 | 42.3452381 | Hyper-methylated | PCa\|NT DMRs |
| chr8 | 21263846 | 21275095 | 0.001969641 | −39.75170068 | Hypo-methylated | PCa\|NT DMRs |
| chr8 | 21614351 | 21626112 | 0.000815653 | −22.51772487 | Hypo-methylated | PCa\|NT DMRs |
| chr8 | 21764141 | 21767204 | 3.91E−05 | −39.72063492 | Hypo-methylated | PCa\|NT DMRs |
| chr8 | 21769239 | 21770745 | 0.000198783 | 34.74175824 | Hyper-methylated | PCa\|NT DMRs |
| chr8 | 21900543 | 21906062 | 0.000149006 | 22.24015152 | Hyper-methylated | PCa\|NT DMRs |
| chr8 | 21944035 | 21944339 | 6.94E−06 | 41.34920635 | Hyper-methylated | PCa\|NT DMRs |
| chr8 | 21988910 | 21990525 | 0.000326385 | −22.20337302 | Hypo-methylated | PCa\|NT DMRs |
| chr8 | 21996161 | 21997388 | 0.000139576 | 25.76260504 | Hyper-methylated | PCa\|NT DMRs |
| chr8 | 22021493 | 22021814 | 0.0014802 | 20.23933747 | Hyper-methylated | PCa\|NT DMRs |
| chr8 | 22429857 | 22436387 | 0.004556523 | 23.52857143 | Hyper-methylated | PCa\|NT DMRs |
| chr8 | 22437343 | 22438902 | 0.002055421 | 33.99383754 | Hyper-methylated | PCa\|NT DMRs |
| chr8 | 22442766 | 22447432 | 2.72E−07 | −41.2665733 | Hypo-methylated | PCa\|NT DMRs |
| chr8 | 22547685 | 22548978 | 6.51E−11 | 36.50228175 | Hyper-methylated | PCa\|NT DMRs |
| chr8 | 22559691 | 22571369 | 8.70E−08 | 23.43117386 | Hyper-methylated | PCa\|NT DMRs |
| chr8 | 22605261 | 22605358 | 0.007959104 | 21.22040816 | Hyper-methylated | PCa\|NT DMRs |
| chr8 | 22626344 | 22632950 | 0.000132819 | −23.44179894 | Hypo-methylated | PCa\|NT DMRs |
| chr8 | 23146336 | 23150322 | 0.002585277 | −30.90535714 | Hypo-methylated | PCa\|NT DMRs |
| chr8 | 23260500 | 23261067 | 1.00E−11 | 31.93865546 | Hyper-methylated | PCa\|NT DMRs |
| chr8 | 24529557 | 24529811 | 0.005968871 | −37.31428571 | Hypo-methylated | PCa\|NT DMRs |
| chr8 | 25105815 | 25106146 | 0.001042833 | −32.54285714 | Hypo-methylated | PCa\|NT DMRs |
| chr8 | 25678812 | 25681778 | 8.35E−08 | −32.84761905 | Hypo-methylated | PCa\|NT DMRs |
| chr8 | 26047518 | 26047944 | 5.27E−07 | 22.40326531 | Hyper-methylated | PCa\|NT DMRs |
| chr8 | 26060747 | 26062632 | 0.001896858 | −35.30899471 | Hypo-methylated | PCa\|NT DMRs |
| chr8 | 26295201 | 26298861 | 6.50E−07 | 46.72444444 | Hyper-methylated | PCa\|NT DMRs |
| chr8 | 26314979 | 26323381 | 0.001969641 | −47.42857143 | Hypo-methylated | PCa\|NT DMRs |
| chr8 | 26434373 | 26434423 | 0.004057568 | 20.54761905 | Hyper-methylated | PCa\|NT DMRs |
| chr8 | 26476734 | 26487972 | 0.000328483 | −23.34603175 | Hypo-methylated | PCa\|NT DMRs |
| chr8 | 27141129 | 27145133 | 0.001397404 | 25.37428571 | Hyper-methylated | PCa\|NT DMRs |
| chr8 | 27331576 | 27337571 | 0.00379014 | −29.76190476 | Hypo-methylated | PCa\|NT DMRs |
| chr8 | 27441261 | 27441384 | 0.004095673 | −45.68253968 | Hypo-methylated | PCa\|NT DMRs |
| chr8 | 27467666 | 27469002 | 0.002767976 | 35.57272727 | Hyper-methylated | PCa\|NT DMRs |
| chr8 | 28051550 | 28051712 | 0.000836132 | 35.47671958 | Hyper-methylated | PCa\|NT DMRs |
| chr8 | 28636837 | 28637198 | 1.45E−06 | 45.50113379 | Hyper-methylated | PCa\|NT DMRs |
| chr8 | 29205513 | 29205929 | 3.20E−05 | 24.87619048 | Hyper-methylated | PCa\|NT DMRs |
| chr8 | 30819100 | 30860251 | 0.000974795 | −33.19345238 | Hypo-methylated | PCa\|NT DMRs |
| chr8 | 31072942 | 31100884 | 1.19E−05 | −26.08857143 | Hypo-methylated | PCa\|NT DMRs |
| chr8 | 32083616 | 32394396 | 9.19E−10 | −39.67403994 | Hypo-methylated | PCa\|NT DMRs |
| chr8 | 32435759 | 32985289 | 3.17E−12 | −39.2018315 | Hypo-methylated | PCa\|NT DMRs |
| chr8 | 33432197 | 33432618 | 0.001789867 | 27.39153439 | Hyper-methylated | PCa\|NT DMRs |
| chr8 | 36064877 | 36530119 | 5.49E−16 | −38.4218254 | Hypo-methylated | PCa\|NT DMRs |
| chr8 | 37082339 | 37082626 | 2.20E−08 | 35.6140056 | Hyper-methylated | PCa\|NT DMRs |
| chr8 | 37554902 | 37561253 | 1.01E−20 | 26.84407369 | Hyper-methylated | PCa\|NT DMRs |
| chr8 | 37605454 | 37605718 | 0.008482405 | 27.42857143 | Hyper-methylated | PCa\|NT DMRs |
| chr8 | 37692789 | 37696672 | 0.001279232 | −21.61640212 | Hypo-methylated | PCa\|NT DMRs |
| chr8 | 37698692 | 37698866 | 0.000860601 | 28 | Hyper-methylated | PCa\|NT DMRs |
| chr8 | 37699124 | 37700577 | 5.68E−21 | −22.48994048 | Hypo-methylated | PCa\|NT DMRs |
| chr8 | 37793533 | 37797680 | 0.000513415 | −23.73809524 | Hypo-methylated | PCa\|NT DMRs |
| chr8 | 38287269 | 38287486 | 0.000205174 | 25.09722222 | Hyper-methylated | PCa\|NT DMRs |
| chr8 | 38494742 | 38508706 | 0.000118965 | 21.47979798 | Hyper-methylated | PCa\|NT DMRs |
| chr8 | 38586772 | 38592424 | 0.00903722 | −22.7 | Hypo-methylated | PCa\|NT DMRs |
| chr8 | 38757333 | 38757879 | 1.37E−09 | 47.41393298 | Hyper-methylated | PCa\|NT DMRs |
| chr8 | 38965081 | 38965322 | 2.80E−09 | 24.47252747 | Hyper-methylated | PCa\|NT DMRs |
| chr8 | 41400789 | 41416396 | 8.17E−05 | −50.13852814 | Hypo-methylated | PCa\|NT DMRs |
| chr8 | 41423303 | 41425291 | 1.86E−05 | 42.4057971 | Hyper-methylated | PCa\|NT DMRs |
| chr8 | 41506810 | 41507551 | 0.000176046 | −21.15757576 | Hypo-methylated | PCa\|NT DMRs |
| chr8 | 41522584 | 41523993 | 0.00037547 | −32.72349206 | Hypo-methylated | PCa\|NT DMRs |
| chr8 | 41540512 | 41541906 | 0.004288989 | 27.41456583 | Hyper-methylated | PCa\|NT DMRs |
| chr8 | 41640907 | 41670474 | 0.005429387 | 27.303125 | Hyper-methylated | PCa\|NT DMRs |
| chr8 | 42154670 | 42154952 | 0.005968871 | −42.1552381 | Hypo-methylated | PCa\|NT DMRs |
| chr8 | 42328772 | 42329926 | 0.003871035 | −51.67321429 | Hypo-methylated | PCa\|NT DMRs |
| chr8 | 42571805 | 42580051 | 0.002094 | −28.13214286 | Hypo-methylated | PCa\|NT DMRs |
| chr8 | 42660980 | 42662690 | 0.009407512 | −37.80687831 | Hypo-methylated | PCa\|NT DMRs |
| chr8 | 42922954 | 42923136 | 0.001969641 | −50.47619048 | Hypo-methylated | PCa\|NT DMRs |
| chr8 | 49308856 | 49309427 | 0.000136933 | 35.66369048 | Hyper-methylated | PCa\|NT DMRs |
| chr8 | 49485249 | 49495212 | 2.28E−14 | −53.23150963 | Hypo-methylated | PCa\|NT DMRs |
| chr8 | 50920607 | 51383421 | 2.00E−20 | −34.0789011 | Hypo-methylated | PCa\|NT DMRs |
| chr8 | 51767499 | 51787314 | 6.42E−05 | −28.62169312 | Hypo-methylated | PCa\|NT DMRs |
| chr8 | 54789846 | 54792185 | 1.46E−16 | 37.08552754 | Hyper-methylated | PCa\|NT DMRs |
| chr8 | 55300553 | 55338708 | 4.45E−08 | −35.42126984 | Hypo-methylated | PCa\|NT DMRs |
| chr8 | 58375388 | 58392688 | 0.006410012 | 24.82900433 | Hyper-methylated | PCa\|NT DMRs |
| chr8 | 61176666 | 61183652 | 0.007560755 | −41.16369048 | Hypo-methylated | PCa\|NT DMRs |
| chr8 | 62721086 | 62887070 | 0.000826653 | −25.34017857 | Hypo-methylated | PCa\|NT DMRs |
| chr8 | 62975784 | 63092355 | 6.36E−11 | −29.25434174 | Hypo-methylated | PCa\|NT DMRs |
| chr8 | 65499369 | 65499825 | 5.94E−06 | 47.97186147 | Hyper-methylated | PCa\|NT DMRs |
| chr8 | 65711626 | 65711691 | 0.000205174 | 44.84821429 | Hyper-methylated | PCa\|NT DMRs |
| chr8 | 67024862 | 67025042 | 0.000831781 | 21.35238095 | Hyper-methylated | PCa\|NT DMRs |
| chr8 | 68733543 | 68733639 | 0.005968871 | −40.75333333 | Hypo-methylated | PCa\|NT DMRs |
| chr8 | 70744312 | 70745250 | 0.000117699 | 21.05612245 | Hyper-methylated | PCa\|NT DMRs |
| chr8 | 70944891 | 70947091 | 1.25E−06 | 20.34933333 | Hyper-methylated | PCa\|NT DMRs |
| chr8 | 71909255 | 72392773 | 1.01E−08 | −38.1212585 | Hypo-methylated | PCa\|NT DMRs |
| chr8 | 72924085 | 73151529 | 1.03E−07 | −38.28246753 | Hypo-methylated | PCa\|NT DMRs |
| chr8 | 74332435 | 74339512 | 0.000396946 | −32.76011905 | Hypo-methylated | PCa\|NT DMRs |
| chr8 | 74660057 | 74660428 | 0.001969641 | 27.88492063 | Hyper-methylated | PCa\|NT DMRs |
| chr8 | 79899956 | 79945510 | 5.94E−06 | −34.44588745 | Hypo-methylated | PCa\|NT DMRs |
| chr8 | 79982432 | 80212085 | 3.19E−07 | −33.47380952 | Hypo-methylated | PCa\|NT DMRs |
| chr8 | 80255996 | 80518425 | 1.57E−08 | −33.50419501 | Hypo-methylated | PCa\|NT DMRs |
| chr8 | 81806094 | 81806180 | 0.000957078 | 25.03571429 | Hyper-methylated | PCa\|NT DMRs |
| chr8 | 82543201 | 82543456 | 1.52E−07 | 51.25034014 | Hyper-methylated | PCa\|NT DMRs |
| chr8 | 83572888 | 83791937 | 1.43E−05 | −20.7210084 | Hypo-methylated | PCa\|NT DMRs |
| chr8 | 84053134 | 84164157 | 1.16E−06 | −32.70535714 | Hypo-methylated | PCa\|NT DMRs |
| chr8 | 84470335 | 84598492 | 0.000364072 | −22.97985348 | Hypo-methylated | PCa\|NT DMRs |
| chr8 | 84642023 | 84844029 | 1.87E−09 | −27.3543514 | Hypo-methylated | PCa\|NT DMRs |
| chr8 | 86350545 | 86351196 | 4.43E−10 | 33.51890332 | Hyper-methylated | PCa\|NT DMRs |
| chr8 | 87081679 | 87081820 | 4.98E−06 | 33.8115942 | Hyper-methylated | PCa\|NT DMRs |
| chr8 | 87648629 | 88037918 | 4.97E−13 | −33.69057337 | Hypo-methylated | PCa\|NT DMRs |
| chr8 | 88085853 | 89250760 | 7.81E−43 | −33.8699232 | Hypo-methylated | PCa\|NT DMRs |
| chr8 | 89369973 | 89872954 | 2.63E−12 | −37.39082462 | Hypo-methylated | PCa\|NT DMRs |
| chr8 | 90547495 | 90547562 | 0.000974795 | −20.44642857 | Hypo-methylated | PCa\|NT DMRs |
| chr8 | 91109576 | 91488000 | 9.17E−09 | −37.21071429 | Hypo-methylated | PCa\|NT DMRs |
| chr8 | 94508105 | 94508296 | 4.90E−08 | 33.83896104 | Hyper-methylated | PCa\|NT DMRs |
| chr8 | 96085404 | 96085917 | 4.53E−09 | 44.45402299 | Hyper-methylated | PCa\|NT DMRs |
| chr8 | 97156648 | 97172784 | 2.52E−46 | 23.67092829 | Hyper-methylated | PCa\|NT DMRs |
| chr8 | 97173592 | 97174041 | 0.002437742 | 31.68253968 | Hyper-methylated | PCa\|NT DMRs |
| chr8 | 98271656 | 98271950 | 0.001969641 | 44.23015873 | Hyper-methylated | PCa\|NT DMRs |
| chr8 | 98617520 | 98619242 | 0.000305995 | −21.7952381 | Hypo-methylated | PCa\|NT DMRs |
| chr8 | 99951787 | 99953050 | 1.11E−25 | 40.70776644 | Hyper-methylated | PCa\|NT DMRs |
| chr8 | 101118292 | 101118744 | 8.86E−09 | 21.7537594 | Hyper-methylated | PCa\|NT DMRs |
| chr8 | 101821841 | 101822591 | 0.005672841 | 53.50238095 | Hyper-methylated | PCa\|NT DMRs |
| chr8 | 102236113 | 102236447 | 1.67E−06 | 51.75210084 | Hyper-methylated | PCa\|NT DMRs |
| chr8 | 102373999 | 102404615 | 1.97E−05 | −44.41047619 | Hypo-methylated | PCa\|NT DMRs |
| chr8 | 102431303 | 102450992 | 3.75E−05 | −52.6172619 | Hypo-methylated | PCa\|NT DMRs |
| chr8 | 103668997 | 103670101 | 0.000455672 | 29.63690476 | Hyper-methylated | PCa\|NT DMRs |
| chr8 | 103671436 | 103698389 | 0.005968871 | −26.67619048 | Hypo-methylated | PCa\|NT DMRs |
| chr8 | 103750821 | 103751040 | 1.42E−05 | 26.43707483 | Hyper-methylated | PCa\|NT DMRs |
| chr8 | 105342233 | 105342450 | 0.001115302 | 49.48809524 | Hyper-methylated | PCa\|NT DMRs |
| chr8 | 105379665 | 105379943 | 0.001254235 | 25.10622711 | Hyper-methylated | PCa\|NT DMRs |
| chr8 | 108579034 | 108601690 | 6.96E−08 | −30.73022774 | Hypo-methylated | PCa\|NT DMRs |
| chr8 | 109799167 | 109799880 | 9.88E−15 | 23.92774327 | Hyper-methylated | PCa\|NT DMRs |
| chr8 | 111600692 | 112053213 | 3.50E−24 | −32.73809524 | Hypo-methylated | PCa\|NT DMRs |
| chr8 | 112065032 | 112663088 | 1.33E−25 | −29.19802956 | Hypo-methylated | PCa\|NT DMRs |
| chr8 | 115228664 | 115332842 | 3.60E−14 | −28.77045455 | Hypo-methylated | PCa\|NT DMRs |
| chr8 | 115530086 | 115614442 | 2.01E−07 | −37.82595238 | Hypo-methylated | PCa\|NT DMRs |
| chr8 | 115887740 | 116071882 | 1.10E−13 | −37.18809524 | Hypo-methylated | PCa\|NT DMRs |
| chr8 | 117991719 | 118023209 | 3.66E−05 | −25.53333333 | Hypo-methylated | PCa\|NT DMRs |
| chr8 | 118632824 | 118785270 | 6.94E−06 | −36.28507937 | Hypo-methylated | PCa\|NT DMRs |
| chr8 | 120685356 | 120694752 | 1.48E−06 | −24.65194805 | Hypo-methylated | PCa\|NT DMRs |
| chr8 | 120779442 | 120779900 | 0.00569382 | −33.87 | Hypo-methylated | PCa\|NT DMRs |
| chr8 | 121137120 | 121138498 | 2.35E−05 | 32.21889401 | Hyper-methylated | PCa\|NT DMRs |
| chr8 | 123928248 | 123928529 | 0.005968871 | −26.21714286 | Hypo-methylated | PCa\|NT DMRs |
| chr8 | 124173160 | 124173634 | 1.68E−06 | 26.69568452 | Hyper-methylated | PCa\|NT DMRs |
| chr8 | 124194759 | 124195440 | 0.009379999 | 21.375 | Hyper-methylated | PCa\|NT DMRs |
| chr8 | 126737168 | 127493574 | 2.75E−16 | −32.96657682 | Hypo-methylated | PCa\|NT DMRs |
| chr8 | 127889338 | 127889425 | 0.005968871 | −30.94285714 | Hypo-methylated | PCa\|NT DMRs |
| chr8 | 128824018 | 128826584 | 0.00064571 | 37.52721088 | Hyper-methylated | PCa\|NT DMRs |
| chr8 | 128923716 | 128923775 | 0.004095673 | −42.14285714 | Hypo-methylated | PCa\|NT DMRs |
| chr8 | 129165439 | 129165636 | 0.00064571 | −62.44897959 | Hypo-methylated | PCa\|NT DMRs |
| chr8 | 129771078 | 129807059 | 4.34E−05 | −45.92380952 | Hypo-methylated | PCa\|NT DMRs |
| chr8 | 129870344 | 130645891 | 4.90E−17 | −33.64317998 | Hypo-methylated | PCa\|NT DMRs |
| chr8 | 130708576 | 130848921 | 0.001612399 | −23.10076805 | Hypo-methylated | PCa\|NT DMRs |
| chr8 | 131000521 | 131010839 | 0.006743251 | 28.3015873 | Hyper-methylated | PCa\|NT DMRs |
| chr8 | 131455250 | 131455494 | 4.27E−05 | 20.739599 | Hyper-methylated | PCa\|NT DMRs |
| chr8 | 131475677 | 131476021 | 0.000836132 | −41.24761905 | Hypo-methylated | PCa\|NT DMRs |
| chr8 | 131610370 | 131933793 | 1.57E−09 | −32.36728778 | Hypo-methylated | PCa\|NT DMRs |
| chr8 | 132133320 | 132257632 | 6.42E−05 | −42.98941799 | Hypo-methylated | PCa\|NT DMRs |
| chr8 | 133707885 | 133769795 | 9.76E−07 | −28.08058608 | Hypo-methylated | PCa\|NT DMRs |
| chr8 | 134498266 | 134513593 | 4.37E−05 | 48.26696429 | Hyper-methylated | PCa\|NT DMRs |
| chr8 | 135194427 | 135306812 | 2.06E−05 | −39.13401361 | Hypo-methylated | PCa\|NT DMRs |
| chr8 | 135338267 | 135465459 | 9.32E−06 | −25.83571429 | Hypo-methylated | PCa\|NT DMRs |
| chr8 | 135919142 | 135973097 | 6.74E−11 | −32.48108466 | Hypo-methylated | PCa\|NT DMRs |
| chr8 | 136092361 | 136449001 | 1.86E−10 | −30.08013937 | Hypo-methylated | PCa\|NT DMRs |
| chr8 | 137912402 | 139158455 | 9.24E−38 | −36.98143226 | Hypo-methylated | PCa\|NT DMRs |
| chr8 | 139672296 | 139672680 | 0.00068166 | 27.98511905 | Hyper-methylated | PCa\|NT DMRs |
| chr8 | 139870105 | 139871676 | 0.001181579 | −28.85170068 | Hypo-methylated | PCa\|NT DMRs |
| chr8 | 139972115 | 140630754 | 4.37E−28 | −34.84794974 | Hypo-methylated | PCa\|NT DMRs |
| chr8 | 140636481 | 140642897 | 7.35E−05 | −33.21428571 | Hypo-methylated | PCa\|NT DMRs |
| chr8 | 141052553 | 141053137 | 0.001496387 | −34.99859944 | Hypo-methylated | PCa\|NT DMRs |
| chr8 | 141100648 | 141100718 | 0.005968871 | −46.48571429 | Hypo-methylated | PCa\|NT DMRs |
| chr8 | 141108104 | 141108690 | 0.00218362 | 21.01820728 | Hyper-methylated | PCa\|NT DMRs |
| chr8 | 141354760 | 141361215 | 0.000475166 | 21.67216117 | Hyper-methylated | PCa\|NT DMRs |
| chr8 | 141568364 | 141568869 | 0.001198104 | 31.32653061 | Hyper-methylated | PCa\|NT DMRs |
| chr8 | 142095375 | 142095826 | 0.000122432 | −23.78354978 | Hypo-methylated | PCa\|NT DMRs |
| chr8 | 142096025 | 142097924 | 0.009106413 | 47.33571429 | Hyper-methylated | PCa\|NT DMRs |
| chr8 | 142119306 | 142119501 | 0.000775154 | 29.23809524 | Hyper-methylated | PCa\|NT DMRs |
| chr8 | 142248919 | 142249149 | 0.001969641 | 60.97619048 | Hyper-methylated | PCa\|NT DMRs |
| chr8 | 142270147 | 142274529 | 0.000513415 | −29.12346939 | Hypo-methylated | PCa\|NT DMRs |
| chr8 | 142336649 | 142339724 | 6.90E−08 | 32.72210884 | Hyper-methylated | PCa\|NT DMRs |
| chr8 | 142388044 | 142389061 | 0.009924186 | −38.62010582 | Hypo-methylated | PCa\|NT DMRs |
| chr8 | 142402682 | 142402949 | 0.00358897 | −20.62507937 | Hypo-methylated | PCa\|NT DMRs |
| chr8 | 142409106 | 142409703 | 3.44E−06 | −50.375 | Hypo-methylated | PCa\|NT DMRs |
| chr8 | 142520479 | 142523003 | 0.001346417 | −27.49801587 | Hypo-methylated | PCa\|NT DMRs |
| chr8 | 142538775 | 142545237 | 0.001064034 | −26.52312925 | Hypo-methylated | PCa\|NT DMRs |
| chr8 | 142555472 | 142571998 | 0.007670329 | −20.7464986 | Hypo-methylated | PCa\|NT DMRs |
| chr8 | 142577155 | 142590916 | 5.56E−05 | −26.45844156 | Hypo-methylated | PCa\|NT DMRs |
| chr8 | 142592894 | 142593783 | 0.000672633 | −22.68571429 | Hypo-methylated | PCa\|NT DMRs |
| chr8 | 142611236 | 142626664 | 1.64E−08 | −24.73066667 | Hypo-methylated | PCa\|NT DMRs |
| chr8 | 142690362 | 142690771 | 0.001152293 | −29.25460317 | Hypo-methylated | PCa\|NT DMRs |
| chr8 | 142692497 | 142719236 | 2.11E−11 | −22.89033816 | Hypo-methylated | PCa\|NT DMRs |
| chr8 | 142834497 | 142839706 | 3.10E−06 | −27.42212885 | Hypo-methylated | PCa\|NT DMRs |
| chr8 | 142984467 | 142986451 | 0.004553481 | −43.14285714 | Hypo-methylated | PCa\|NT DMRs |
| chr8 | 143085128 | 143085663 | 5.19E−07 | −51.40842491 | Hypo-methylated | PCa\|NT DMRs |
| chr8 | 143090744 | 143091427 | 1.13E−05 | −26.31648352 | Hypo-methylated | PCa\|NT DMRs |
| chr8 | 143096606 | 143098854 | 1.23E−07 | −21.3584127 | Hypo-methylated | PCa\|NT DMRs |
| chr8 | 143169844 | 143183051 | 4.61E−08 | −24.42787456 | Hypo-methylated | PCa\|NT DMRs |
| chr8 | 143206946 | 143208246 | 7.81E−11 | −33.05791506 | Hypo-methylated | PCa\|NT DMRs |
| chr8 | 143210813 | 143211208 | 1.43E−05 | −26.21680672 | Hypo-methylated | PCa\|NT DMRs |
| chr8 | 143217246 | 143225686 | 2.74E−07 | −25.31105991 | Hypo-methylated | PCa\|NT DMRs |
| chr8 | 143230687 | 143234651 | 0.000298021 | −24.86607143 | Hypo-methylated | PCa\|NT DMRs |
| chr8 | 143235507 | 143235930 | 0.000179253 | −22.68131868 | Hypo-methylated | PCa\|NT DMRs |
| chr8 | 143265415 | 143268430 | 0.002097193 | −48.33469388 | Hypo-methylated | PCa\|NT DMRs |
| chr8 | 143285996 | 143286977 | 0.000205174 | −28.26607143 | Hypo-methylated | PCa\|NT DMRs |
| chr8 | 143307697 | 143314436 | 1.92E−09 | −23.54261905 | Hypo-methylated | PCa\|NT DMRs |
| chr8 | 143329928 | 143331470 | 6.04E−09 | −25.66365079 | Hypo-methylated | PCa\|NT DMRs |
| chr8 | 143342806 | 143343281 | 0.003809353 | −26.52809524 | Hypo-methylated | PCa\|NT DMRs |
| chr8 | 143382202 | 143383762 | 3.39E−07 | 41.1974026 | Hyper-methylated | PCa\|NT DMRs |
| chr8 | 143508582 | 143522040 | 4.33E−06 | −23.89206349 | Hypo-methylated | PCa\|NT DMRs |
| chr8 | 143544017 | 143546524 | 3.54E−18 | 21.94169001 | Hyper-methylated | PCa\|NT DMRs |
| chr8 | 143573328 | 143574583 | 0.000250214 | −21.71071429 | Hypo-methylated | PCa\|NT DMRs |
| chr8 | 143624954 | 143626403 | 1.10E−07 | −43.56037415 | Hypo-methylated | PCa\|NT DMRs |
| chr8 | 143630963 | 143632473 | 6.20E−05 | −20.81972789 | Hypo-methylated | PCa\|NT DMRs |
| chr8 | 143656202 | 143656372 | 0.007959104 | −21.84013605 | Hypo-methylated | PCa\|NT DMRs |
| chr8 | 143700112 | 143700184 | 0.007959104 | −27.48979592 | Hypo-methylated | PCa\|NT DMRs |
| chr8 | 143762028 | 143762485 | 0.00179283 | 30.07460317 | Hyper-methylated | PCa\|NT DMRs |
| chr8 | 143832005 | 143832527 | 0.007552382 | 20.72321429 | Hyper-methylated | PCa\|NT DMRs |
| chr8 | 143850699 | 143851600 | 1.46E−06 | −40.96825397 | Hypo-methylated | PCa\|NT DMRs |
| chr8 | 143977598 | 143984504 | 0.000111427 | −23.96666667 | Hypo-methylated | PCa\|NT DMRs |
| chr8 | 144105161 | 144108519 | 7.12E−11 | 44.30995671 | Hyper-methylated | PCa\|NT DMRs |
| chr8 | 144118211 | 144125253 | 0.000544917 | −22.93792517 | Hypo-methylated | PCa\|NT DMRs |
| chr8 | 144178766 | 144188067 | 0.00064571 | −37.2244898 | Hypo-methylated | PCa\|NT DMRs |
| chr8 | 144265064 | 144268735 | 4.03E−09 | −35.02903828 | Hypo-methylated | PCa\|NT DMRs |
| chr8 | 144283322 | 144283354 | 0.005968871 | 31.88952381 | Hyper-methylated | PCa\|NT DMRs |
| chr8 | 144371506 | 144371553 | 0.001969641 | 28.46825397 | Hyper-methylated | PCa\|NT DMRs |
| chr8 | 144442847 | 144443233 | 0.001969641 | 22.05208333 | Hyper-methylated | PCa\|NT DMRs |
| chr8 | 144464615 | 144465299 | 0.000980855 | −36.82653061 | Hypo-methylated | PCa\|NT DMRs |
| chr8 | 144476461 | 144476530 | 0.001115302 | −25.94642857 | Hypo-methylated | PCa\|NT DMRs |
| chr8 | 144480110 | 144480867 | 3.20E−05 | 31.3139881 | Hyper-methylated | PCa\|NT DMRs |
| chr8 | 144481225 | 144482659 | 3.68E−08 | −27.29723502 | Hypo-methylated | PCa\|NT DMRs |
| chr8 | 144487603 | 144488145 | 0.001139334 | −35.14285714 | Hypo-methylated | PCa\|NT DMRs |
| chr8 | 144628719 | 144635319 | 0.005800294 | 21.60266667 | Hyper-methylated | PCa\|NT DMRs |
| chr8 | 144644321 | 144645084 | 0.006410012 | −23.57857143 | Hypo-methylated | PCa\|NT DMRs |
| chr8 | 144655447 | 144656066 | 0.001181579 | 45.31972789 | Hyper-methylated | PCa\|NT DMRs |
| chr8 | 144666236 | 144666445 | 0.008482405 | 40.35714286 | Hyper-methylated | PCa\|NT DMRs |
| chr8 | 144680875 | 144681950 | 0.000111427 | −25.23816327 | Hypo-methylated | PCa\|NT DMRs |
| chr8 | 144800968 | 144801303 | 3.47E−05 | −33.60918367 | Hypo-methylated | PCa\|NT DMRs |
| chr8 | 144809795 | 144810716 | 2.21E−20 | 29.54041812 | Hyper-methylated | PCa\|NT DMRs |
| chr8 | 144852195 | 144853210 | 0.000159126 | 22.23809524 | Hyper-methylated | PCa\|NT DMRs |
| chr8 | 144883852 | 144884746 | 0.004231599 | −32.10622711 | Hypo-methylated | PCa\|NT DMRs |
| chr8 | 144895169 | 144895870 | 0.000308231 | −23.54428571 | Hypo-methylated | PCa\|NT DMRs |
| chr8 | 144896231 | 144896410 | 0.002804846 | 33.82440476 | Hyper-methylated | PCa\|NT DMRs |
| chr8 | 144923621 | 144923673 | 0.000205174 | −24.21428571 | Hypo-methylated | PCa\|NT DMRs |
| chr8 | 144964837 | 144965646 | 0.000492616 | 27.80761905 | Hyper-methylated | PCa\|NT DMRs |
| chr8 | 144978099 | 144980365 | 0.001969641 | −47.42857143 | Hypo-methylated | PCa\|NT DMRs |
| chr8 | 145003406 | 145004388 | 2.63E−12 | 30.66631823 | Hyper-methylated | PCa\|NT DMRs |
| chr8 | 145006178 | 145007582 | 1.16E−10 | −23.21276596 | Hypo-methylated | PCa\|NT DMRs |
| chr8 | 145010401 | 145011617 | 0.003764982 | 26.39087302 | Hyper-methylated | PCa\|NT DMRs |
| chr8 | 145012905 | 145014527 | 2.17E−08 | 26.32010582 | Hyper-methylated | PCa\|NT DMRs |
| chr8 | 145022596 | 145022790 | 0.005968871 | 36.85142857 | Hyper-methylated | PCa\|NT DMRs |
| chr8 | 145052060 | 145054037 | 3.16E−07 | 44.11349206 | Hyper-methylated | PCa\|NT DMRs |
| chr8 | 145056465 | 145059928 | 1.83E−06 | −26.28018433 | Hypo-methylated | PCa\|NT DMRs |
| chr8 | 145096571 | 145099295 | 0.000250214 | −35.61190476 | Hypo-methylated | PCa\|NT DMRs |
| chr8 | 145129038 | 145129326 | 6.42E−05 | 40.37830688 | Hyper-methylated | PCa\|NT DMRs |
| chr8 | 145162222 | 145162641 | 0.000474458 | −42.04395604 | Hypo-methylated | PCa\|NT DMRs |
| chr8 | 145237052 | 145237263 | 0.005968871 | −55.96 | Hypo-methylated | PCa\|NT DMRs |
| chr8 | 145550987 | 145551177 | 1.62E−05 | 22.66809524 | Hyper-methylated | PCa\|NT DMRs |
| chr8 | 145577298 | 145577849 | 0.006320534 | 20.82619048 | Hyper-methylated | PCa\|NT DMRs |
| chr8 | 145578008 | 145578410 | 2.37E−08 | −24.14285714 | Hypo-methylated | PCa\|NT DMRs |
| chr8 | 145646575 | 145649007 | 0.004893702 | −20.65555556 | Hypo-methylated | PCa\|NT DMRs |
| chr8 | 145651397 | 145651575 | 0.004095673 | 38.16666667 | Hyper-methylated | PCa\|NT DMRs |
| chr8 | 145688603 | 145690550 | 0.00530467 | −20.52167488 | Hypo-methylated | PCa\|NT DMRs |
| chr8 | 145742341 | 145742509 | 3.52E−05 | −25.88205128 | Hypo-methylated | PCa\|NT DMRs |
| chr8 | 145749825 | 145752311 | 4.35E−09 | −23.55730428 | Hypo-methylated | PCa\|NT DMRs |
| chr8 | 145753330 | 145753922 | 0.000254837 | −27.69929453 | Hypo-methylated | PCa\|NT DMRs |
| chr8 | 145755264 | 145756232 | 8.06E−11 | 34.89721254 | Hyper-methylated | PCa\|NT DMRs |
| chr8 | 146253101 | 146253149 | 0.002097193 | −31.34285714 | Hypo-methylated | PCa\|NT DMRs |
| chr9 | 83408 | 83463 | 0.005968871 | −24.2952381 | Hypo-methylated | PCa\|NT DMRs |
| chr9 | 446271 | 446461 | 0.003871035 | 31.19642857 | Hyper-methylated | PCa\|NT DMRs |
| chr9 | 804498 | 811483 | 0.005295761 | −31.46103896 | Hypo-methylated | PCa\|NT DMRs |
| chr9 | 969611 | 970971 | 1.05E−06 | 21.11133005 | Hyper-methylated | PCa\|NT DMRs |
| chr9 | 5206951 | 5327317 | 0.000370938 | −24.56493506 | Hypo-methylated | PCa\|NT DMRs |
| chr9 | 5841748 | 5841963 | 0.00064571 | 47.04081633 | Hyper-methylated | PCa\|NT DMRs |
| chr9 | 6412491 | 6412687 | 0.000191863 | 29.03720238 | Hyper-methylated | PCa\|NT DMRs |
| chr9 | 6703571 | 6703660 | 0.001969641 | 22.98412698 | Hyper-methylated | PCa\|NT DMRs |
| chr9 | 7879923 | 8331761 | 1.14E−07 | −35.46634921 | Hypo-methylated | PCa\|NT DMRs |
| chr9 | 9032714 | 10195094 | 2.97E−21 | −34.10028011 | Hypo-methylated | PCa\|NT DMRs |
| chr9 | 14199285 | 14203629 | 0.000783791 | −49.77857143 | Hypo-methylated | PCa\|NT DMRs |
| chr9 | 14343048 | 14350085 | 6.25E−10 | 35.15319396 | Hyper-methylated | PCa\|NT DMRs |
| chr9 | 17018011 | 17018256 | 0.00064571 | −30.19251701 | Hypo-methylated | PCa\|NT DMRs |
| chr9 | 21138266 | 21235576 | 0.00026034 | −27.5639881 | Hypo-methylated | PCa\|NT DMRs |
| chr9 | 21971249 | 21974592 | 0.000822388 | 28.58441558 | Hyper-methylated | PCa\|NT DMRs |
| chr9 | 21976437 | 21993697 | 0.000149287 | 35.68907563 | Hyper-methylated | PCa\|NT DMRs |
| chr9 | 23820974 | 23821145 | 5.30E−07 | 21.46469622 | Hyper-methylated | PCa\|NT DMRs |
| chr9 | 23824191 | 23824651 | 6.69E−05 | 31.17142857 | Hyper-methylated | PCa\|NT DMRs |
| chr9 | 24897540 | 25293313 | 5.20E−08 | −28.56365915 | Hypo-methylated | PCa\|NT DMRs |
| chr9 | 25836072 | 26315147 | 1.17E−15 | −32.91326531 | Hypo-methylated | PCa\|NT DMRs |
| chr9 | 26664519 | 26664650 | 5.94E−06 | −28.37748918 | Hypo-methylated | PCa\|NT DMRs |
| chr9 | 27690811 | 28476568 | 9.52E−23 | −34.79254902 | Hypo-methylated | PCa\|NT DMRs |
| chr9 | 28524474 | 29135328 | 1.75E−17 | −35.78165266 | Hypo-methylated | PCa\|NT DMRs |
| chr9 | 29215194 | 29721755 | 1.21E−08 | −31.36306306 | Hypo-methylated | PCa\|NT DMRs |
| chr9 | 31160895 | 31160979 | 0.005968871 | −40.49809524 | Hypo-methylated | PCa\|NT DMRs |
| chr9 | 31262106 | 31570099 | 2.41E−08 | −30.47329193 | Hypo-methylated | PCa\|NT DMRs |
| chr9 | 31808482 | 32369843 | 8.62E−09 | −25.61503759 | Hypo-methylated | PCa\|NT DMRs |
| chr9 | 32597417 | 32742987 | 2.62E−09 | −34.24039409 | Hypo-methylated | PCa\|NT DMRs |
| chr9 | 32817548 | 32876791 | 2.03E−06 | −39.33206349 | Hypo-methylated | PCa\|NT DMRs |
| chr9 | 32955754 | 32956314 | 1.29E−08 | 26.94380952 | Hyper-methylated | PCa\|NT DMRs |
| chr9 | 33167845 | 33169708 | 0.000729529 | 30.48120301 | Hyper-methylated | PCa\|NT DMRs |
| chr9 | 33748717 | 33750047 | 1.11E−05 | 47.07575758 | Hyper-methylated | PCa\|NT DMRs |
| chr9 | 33750975 | 33751119 | 1.69E−06 | 35.3929972 | Hyper-methylated | PCa\|NT DMRs |
| chr9 | 34376267 | 34376474 | 0.001344447 | 22.33928571 | Hyper-methylated | PCa\|NT DMRs |
| chr9 | 34379748 | 34379985 | 0.000909347 | 30.37301587 | Hyper-methylated | PCa\|NT DMRs |
| chr9 | 34381268 | 34381893 | 8.74E−05 | 30.80434783 | Hyper-methylated | PCa\|NT DMRs |
| chr9 | 34457260 | 34457898 | 0.004967714 | −20.79802956 | Hypo-methylated | PCa\|NT DMRs |
| chr9 | 34590297 | 34591619 | 6.44E−10 | 36.47135177 | Hyper-methylated | PCa\|NT DMRs |
| chr9 | 34652035 | 34652303 | 4.82E−06 | −20.58184524 | Hypo-methylated | PCa\|NT DMRs |
| chr9 | 35406315 | 35406801 | 0.005641685 | 30.70748299 | Hyper-methylated | PCa\|NT DMRs |
| chr9 | 35645028 | 35646413 | 0.009924186 | 25.46957672 | Hyper-methylated | PCa\|NT DMRs |
| chr9 | 35649721 | 35656719 | 0.002695033 | 25.89732143 | Hyper-methylated | PCa\|NT DMRs |
| chr9 | 35689733 | 35690528 | 3.22E−10 | 32.30334347 | Hyper-methylated | PCa\|NT DMRs |
| chr9 | 35846266 | 35846608 | 0.000314499 | 28.3557423 | Hyper-methylated | PCa\|NT DMRs |
| chr9 | 36038139 | 36038236 | 1.97E−05 | −23.3 | Hypo-methylated | PCa\|NT DMRs |
| chr9 | 36137702 | 36141429 | 0.001969641 | −38.33730159 | Hypo-methylated | PCa\|NT DMRs |
| chr9 | 36458539 | 36458764 | 0.00064571 | 71.5 | Hyper-methylated | PCa\|NT DMRs |
| chr9 | 36765825 | 36781048 | 0.002487133 | 22.17142857 | Hyper-methylated | PCa\|NT DMRs |
| chr9 | 36842684 | 36842757 | 0.005968871 | 44.46190476 | Hyper-methylated | PCa\|NT DMRs |
| chr9 | 37025102 | 37026367 | 3.61E−14 | 22.5452381 | Hyper-methylated | PCa\|NT DMRs |
| chr9 | 37515737 | 37516055 | 0.000117699 | −52.92640693 | Hypo-methylated | PCa\|NT DMRs |
| chr9 | 37938545 | 37938960 | 4.82E−06 | −27.84136905 | Hypo-methylated | PCa\|NT DMRs |
| chr9 | 38242382 | 38253497 | 0.000783791 | −22.90619048 | Hypo-methylated | PCa\|NT DMRs |
| chr9 | 38486933 | 38488409 | 4.09E−09 | 20.33928571 | Hyper-methylated | PCa\|NT DMRs |
| chr9 | 38732661 | 38765566 | 3.66E−05 | −33.9 | Hypo-methylated | PCa\|NT DMRs |
| chr9 | 39062108 | 39062459 | 0.008482405 | −25.88 | Hypo-methylated | PCa\|NT DMRs |
| chr9 | 39132888 | 39132940 | 0.001115302 | 25.5 | Hyper-methylated | PCa\|NT DMRs |
| chr9 | 40408721 | 40601658 | 0.001060348 | −32.50562771 | Hypo-methylated | PCa\|NT DMRs |
| chr9 | 43433835 | 43682005 | 6.23E−06 | −29.98917749 | Hypo-methylated | PCa\|NT DMRs |
| chr9 | 66455995 | 66456414 | 1.45E−06 | 28.44489796 | Hyper-methylated | PCa\|NT DMRs |
| chr9 | 68414144 | 68414200 | 0.003622801 | 30.4047619 | Hyper-methylated | PCa\|NT DMRs |
| chr9 | 69197293 | 69197336 | 0.005968871 | 33.6352381 | Hyper-methylated | PCa\|NT DMRs |
| chr9 | 71222200 | 71226346 | 7.19E−05 | −22.82142857 | Hypo-methylated | PCa\|NT DMRs |
| chr9 | 71735333 | 71735419 | 0.000377317 | −32.25595238 | Hypo-methylated | PCa\|NT DMRs |
| chr9 | 72990148 | 72990275 | 7.57E−05 | −37.38134921 | Hypo-methylated | PCa\|NT DMRs |
| chr9 | 74527382 | 74529776 | 0.000650326 | −42.79960317 | Hypo-methylated | PCa\|NT DMRs |
| chr9 | 74779823 | 74942720 | 4.08E−08 | −25.282493 | Hypo-methylated | PCa\|NT DMRs |
| chr9 | 75533711 | 75696320 | 0.000377317 | −31.91258503 | Hypo-methylated | PCa\|NT DMRs |
| chr9 | 77114273 | 77115745 | 5.01E−08 | 32.87875458 | Hyper-methylated | PCa\|NT DMRs |
| chr9 | 78507195 | 78507575 | 1.98E−07 | 40.86428571 | Hyper-methylated | PCa\|NT DMRs |
| chr9 | 79093254 | 79123427 | 0.005968871 | 21.79047619 | Hyper-methylated | PCa\|NT DMRs |
| chr9 | 79281044 | 79293008 | 0.000117699 | −40.95238095 | Hypo-methylated | PCa\|NT DMRs |
| chr9 | 79620790 | 79629293 | 1.75E−06 | 25.43760263 | Hyper-methylated | PCa\|NT DMRs |
| chr9 | 79631191 | 79631696 | 1.42E−05 | 25.94727891 | Hyper-methylated | PCa\|NT DMRs |
| chr9 | 82590302 | 82590379 | 0.001969641 | −40 | Hypo-methylated | PCa\|NT DMRs |
| chr9 | 84117585 | 84123688 | 0.00044133 | 25.12755102 | Hyper-methylated | PCa\|NT DMRs |
| chr9 | 86047084 | 86047493 | 0.008482405 | −57.9952381 | Hypo-methylated | PCa\|NT DMRs |
| chr9 | 86982507 | 86987097 | 0.003448944 | 29.42857143 | Hyper-methylated | PCa\|NT DMRs |
| chr9 | 87904996 | 87905325 | 6.94E−06 | 45.06190476 | Hyper-methylated | PCa\|NT DMRs |
| chr9 | 88137611 | 88138101 | 5.78E−05 | 35.04325397 | Hyper-methylated | PCa\|NT DMRs |
| chr9 | 88650418 | 88656424 | 0.002097193 | −25.54217687 | Hypo-methylated | PCa\|NT DMRs |
| chr9 | 89763714 | 89763814 | 0.000836132 | 28.83333333 | Hyper-methylated | PCa\|NT DMRs |
| chr9 | 90864438 | 90873839 | 0.000463506 | −46.58008658 | Hypo-methylated | PCa\|NT DMRs |
| chr9 | 91150791 | 91151089 | 4.00E−05 | 20.68831169 | Hyper-methylated | PCa\|NT DMRs |
| chr9 | 91792464 | 91792793 | 0.000132782 | 20.19588745 | Hyper-methylated | PCa\|NT DMRs |
| chr9 | 91897310 | 91911579 | 8.84E−05 | 26.45833333 | Hyper-methylated | PCa\|NT DMRs |
| chr9 | 92045970 | 92050723 | 0.001637131 | −32.74107143 | Hypo-methylated | PCa\|NT DMRs |
| chr9 | 92056190 | 92056419 | 0.00064571 | −42.56462585 | Hypo-methylated | PCa\|NT DMRs |
| chr9 | 92068640 | 92082271 | 0.000652677 | 23.07380952 | Hyper-methylated | PCa\|NT DMRs |
| chr9 | 92254717 | 92255951 | 0.004677148 | 31.48787879 | Hyper-methylated | PCa\|NT DMRs |
| chr9 | 93922036 | 93922272 | 0.000205174 | −24.51845238 | Hypo-methylated | PCa\|NT DMRs |
| chr9 | 94648655 | 94648937 | 0.000118334 | 21.52116402 | Hyper-methylated | PCa\|NT DMRs |
| chr9 | 95727723 | 95728879 | 5.44E−05 | 43.67460317 | Hyper-methylated | PCa\|NT DMRs |
| chr9 | 95799473 | 95799671 | 0.008482405 | 23 | Hyper-methylated | PCa\|NT DMRs |
| chr9 | 95906216 | 95906520 | 0.000974795 | 44.94642857 | Hyper-methylated | PCa\|NT DMRs |
| chr9 | 95964146 | 95965806 | 0.000632596 | −44.12589286 | Hypo-methylated | PCa\|NT DMRs |
| chr9 | 95993869 | 95995565 | 0.005438982 | 23.20714286 | Hyper-methylated | PCa\|NT DMRs |
| chr9 | 96024060 | 96024937 | 4.25E−06 | −25.74260652 | Hypo-methylated | PCa\|NT DMRs |
| chr9 | 96079879 | 96080176 | 0.004418655 | 34.65873016 | Hyper-methylated | PCa\|NT DMRs |
| chr9 | 96104880 | 96105004 | 0.004095673 | −48.93650794 | Hypo-methylated | PCa\|NT DMRs |
| chr9 | 96212770 | 96212839 | 0.00179283 | −20.17857143 | Hypo-methylated | PCa\|NT DMRs |
| chr9 | 96443290 | 96443600 | 0.002437742 | −34.17619048 | Hypo-methylated | PCa\|NT DMRs |
| chr9 | 96572644 | 96572987 | 1.80E−06 | 26.08015873 | Hyper-methylated | PCa\|NT DMRs |
| chr9 | 96670941 | 96671449 | 0.000267383 | 23.82285714 | Hyper-methylated | PCa\|NT DMRs |
| chr9 | 97094722 | 97108504 | 0.002437742 | 26.66666667 | Hyper-methylated | PCa\|NT DMRs |
| chr9 | 97315817 | 97317348 | 2.26E−07 | −44.60698413 | Hypo-methylated | PCa\|NT DMRs |
| chr9 | 97469676 | 97487177 | 5.03E−05 | −20.97238095 | Hypo-methylated | PCa\|NT DMRs |
| chr9 | 98326973 | 98332587 | 0.008138809 | 25.86554622 | Hyper-methylated | PCa\|NT DMRs |
| chr9 | 98514556 | 98514827 | 0.000463436 | 23.85555556 | Hyper-methylated | PCa\|NT DMRs |
| chr9 | 98902244 | 98911780 | 0.004659837 | −27.62337662 | Hypo-methylated | PCa\|NT DMRs |
| chr9 | 99800899 | 99800956 | 0.00068166 | 30.14285714 | Hyper-methylated | PCa\|NT DMRs |
| chr9 | 99838561 | 99840960 | 6.00E−08 | 26.28154762 | Hyper-methylated | PCa\|NT DMRs |
| chr9 | 100069566 | 100069726 | 2.86E−06 | −20.98345865 | Hypo-methylated | PCa\|NT DMRs |
| chr9 | 100337640 | 100337731 | 0.000974795 | 48.61011905 | Hyper-methylated | PCa\|NT DMRs |
| chr9 | 100503347 | 100504015 | 6.33E−13 | 23.05957672 | Hyper-methylated | PCa\|NT DMRs |
| chr9 | 101013423 | 101013498 | 0.004095673 | 51.38095238 | Hyper-methylated | PCa\|NT DMRs |
| chr9 | 101352529 | 101364376 | 0.005968871 | −39.58857143 | Hypo-methylated | PCa\|NT DMRs |
| chr9 | 101378557 | 101446014 | 8.34E−11 | −29.24510582 | Hypo-methylated | PCa\|NT DMRs |
| chr9 | 101761074 | 101761681 | 0.001969641 | −45.53174603 | Hypo-methylated | PCa\|NT DMRs |
| chr9 | 101817650 | 101849787 | 0.001969641 | −21.43401361 | Hypo-methylated | PCa\|NT DMRs |
| chr9 | 102068138 | 102121756 | 2.94E−06 | −32.02970522 | Hypo-methylated | PCa\|NT DMRs |
| chr9 | 102131248 | 102279804 | 0.000205174 | −24.84333333 | Hypo-methylated | PCa\|NT DMRs |
| chr9 | 103447815 | 103447986 | 0.001969641 | −27.4047619 | Hypo-methylated | PCa\|NT DMRs |
| chr9 | 104248322 | 104248763 | 7.59E−06 | 23.42113095 | Hyper-methylated | PCa\|NT DMRs |
| chr9 | 105365772 | 105366360 | 0.003391914 | −24.61164021 | Hypo-methylated | PCa\|NT DMRs |
| chr9 | 106168220 | 106458650 | 2.85E−10 | −27.95935374 | Hypo-methylated | PCa\|NT DMRs |
| chr9 | 106647199 | 106761810 | 5.19E−07 | −25.18595238 | Hypo-methylated | PCa\|NT DMRs |
| chr9 | 107101282 | 107396403 | 9.12E−15 | −31.75291005 | Hypo-methylated | PCa\|NT DMRs |
| chr9 | 109177376 | 109177466 | 0.001969641 | −20.10396825 | Hypo-methylated | PCa\|NT DMRs |
| chr9 | 110249133 | 110249460 | 6.42E−05 | −21.88941799 | Hypo-methylated | PCa\|NT DMRs |
| chr9 | 110252769 | 110253913 | 1.71E−06 | 34.01445578 | Hyper-methylated | PCa\|NT DMRs |
| chr9 | 112231636 | 112240133 | 0.000644025 | −32.92207792 | Hypo-methylated | PCa\|NT DMRs |
| chr9 | 112810291 | 112812848 | 2.75E−18 | 22.33174603 | Hyper-methylated | PCa\|NT DMRs |
| chr9 | 112889589 | 112890061 | 1.76E−06 | 39.8234127 | Hyper-methylated | PCa\|NT DMRs |
| chr9 | 112965233 | 112976082 | 0.007428376 | −23.84102564 | Hypo-methylated | PCa\|NT DMRs |
| chr9 | 113054494 | 113054762 | 1.66E−07 | −22.31298701 | Hypo-methylated | PCa\|NT DMRs |
| chr9 | 113352499 | 113523988 | 1.68E−05 | −29.31755952 | Hypo-methylated | PCa\|NT DMRs |
| chr9 | 114247331 | 114247380 | 0.004095673 | −60.11904762 | Hypo-methylated | PCa\|NT DMRs |
| chr9 | 114392697 | 114392920 | 0.007338757 | −20.25 | Hypo-methylated | PCa\|NT DMRs |
| chr9 | 114654158 | 114654396 | 0.005968871 | −62.5047619 | Hypo-methylated | PCa\|NT DMRs |
| chr9 | 116225452 | 116226372 | 5.25E−18 | −58.43559719 | Hypo-methylated | PCa\|NT DMRs |
| chr9 | 116310993 | 116311238 | 0.000974795 | 43.35714286 | Hyper-methylated | PCa\|NT DMRs |
| chr9 | 116355917 | 116357047 | 0.000847246 | 22.49659864 | Hyper-methylated | PCa\|NT DMRs |
| chr9 | 116378428 | 116378699 | 0.007679542 | 32.57142857 | Hyper-methylated | PCa\|NT DMRs |
| chr9 | 116450226 | 116450453 | 1.13E−07 | 25.25170068 | Hyper-methylated | PCa\|NT DMRs |
| chr9 | 116875626 | 116875740 | 0.001969641 | −58.51428571 | Hypo-methylated | PCa\|NT DMRs |
| chr9 | 116918588 | 116919030 | 0.001924648 | 25.32683983 | Hyper-methylated | PCa\|NT DMRs |
| chr9 | 116973629 | 116982636 | 0.00179283 | −27.81774892 | Hypo-methylated | PCa\|NT DMRs |
| chr9 | 116990117 | 116990215 | 0.003448944 | −27.56802721 | Hypo-methylated | PCa\|NT DMRs |
| chr9 | 117348989 | 117349622 | 0.000238853 | −34.76441103 | Hypo-methylated | PCa\|NT DMRs |
| chr9 | 118917146 | 118919521 | 0.001206208 | 20.45964912 | Hyper-methylated | PCa\|NT DMRs |
| chr9 | 123656845 | 123657587 | 1.60E−11 | −23.13657143 | Hypo-methylated | PCa\|NT DMRs |
| chr9 | 124384800 | 124388584 | 0.001789867 | 34.82592593 | Hyper-methylated | PCa\|NT DMRs |
| chr9 | 124446950 | 124461217 | 0.000237526 | 26.34854071 | Hyper-methylated | PCa\|NT DMRs |
| chr9 | 124955349 | 124955636 | 0.001969641 | −21.36258503 | Hypo-methylated | PCa\|NT DMRs |
| chr9 | 124981272 | 124982930 | 1.90E−12 | 27.61475257 | Hyper-methylated | PCa\|NT DMRs |
| chr9 | 125137516 | 125137660 | 0.005968871 | −24.28571429 | Hypo-methylated | PCa\|NT DMRs |
| chr9 | 126128425 | 126129407 | 0.000960149 | 22.09821429 | Hyper-methylated | PCa\|NT DMRs |
| chr9 | 126440508 | 126440781 | 0.00068166 | −33.59821429 | Hypo-methylated | PCa\|NT DMRs |
| chr9 | 127005260 | 127005444 | 0.001969641 | 25.83730159 | Hyper-methylated | PCa\|NT DMRs |
| chr9 | 127131834 | 127162872 | 0.001844396 | 28.25 | Hyper-methylated | PCa\|NT DMRs |
| chr9 | 127184623 | 127198296 | 3.24E−05 | −21.25935374 | Hypo-methylated | PCa\|NT DMRs |
| chr9 | 127203662 | 127203901 | 8.96E−05 | 40.4202381 | Hyper-methylated | PCa\|NT DMRs |
| chr9 | 127258043 | 127258864 | 7.63E−08 | 36.79315476 | Hyper-methylated | PCa\|NT DMRs |
| chr9 | 127267027 | 127267430 | 1.70E−05 | 24.13659148 | Hyper-methylated | PCa\|NT DMRs |
| chr9 | 127614742 | 127615026 | 0.007560755 | −42.98511905 | Hypo-methylated | PCa\|NT DMRs |
| chr9 | 128091150 | 128091197 | 0.001969641 | −48.02380952 | Hypo-methylated | PCa\|NT DMRs |
| chr9 | 128187841 | 128188016 | 0.001969641 | −51.64285714 | Hypo-methylated | PCa\|NT DMRs |
| chr9 | 128887984 | 128888102 | 0.003622801 | −41.85793651 | Hypo-methylated | PCa\|NT DMRs |
| chr9 | 128928193 | 128932674 | 0.001969641 | −21.08163265 | Hypo-methylated | PCa\|NT DMRs |
| chr9 | 129244998 | 129260546 | 1.59E−06 | 21.36792717 | Hyper-methylated | PCa\|NT DMRs |
| chr9 | 129282457 | 129282630 | 1.97E−05 | −24.7652381 | Hypo-methylated | PCa\|NT DMRs |
| chr9 | 129325429 | 129341093 | 0.001139334 | −21.95238095 | Hypo-methylated | PCa\|NT DMRs |
| chr9 | 129349072 | 129350141 | 0.000632596 | −34.08441558 | Hypo-methylated | PCa\|NT DMRs |
| chr9 | 129410534 | 129417651 | 0.003622801 | 30.09309524 | Hyper-methylated | PCa\|NT DMRs |
| chr9 | 129884295 | 129884762 | 2.04E−05 | 32.12946429 | Hyper-methylated | PCa\|NT DMRs |
| chr9 | 130247532 | 130249679 | 0.001201396 | −20.31111111 | Hypo-methylated | PCa\|NT DMRs |
| chr9 | 130325238 | 130325963 | 0.00044067 | −23.79761905 | Hypo-methylated | PCa\|NT DMRs |
| chr9 | 130461414 | 130461708 | 8.37E−10 | 31.02170868 | Hyper-methylated | PCa\|NT DMRs |
| chr9 | 130498722 | 130504836 | 7.57E−08 | −23.56904762 | Hypo-methylated | PCa\|NT DMRs |
| chr9 | 130538891 | 130545566 | 0.000185577 | 40.24634921 | Hyper-methylated | PCa\|NT DMRs |
| chr9 | 130587934 | 130590839 | 2.53E−07 | −20.125 | Hypo-methylated | PCa\|NT DMRs |
| chr9 | 130615682 | 130616791 | 7.04E−07 | 27.10555556 | Hyper-methylated | PCa\|NT DMRs |
| chr9 | 130636547 | 130636911 | 0.003290817 | −32.60059524 | Hypo-methylated | PCa\|NT DMRs |
| chr9 | 130674451 | 130677049 | 5.38E−14 | −27.46645503 | Hypo-methylated | PCa\|NT DMRs |
| chr9 | 130677075 | 130678966 | 0.001496387 | 31.93109244 | Hyper-methylated | PCa\|NT DMRs |
| chr9 | 130688850 | 130689743 | 4.61E−09 | 27.36959707 | Hyper-methylated | PCa\|NT DMRs |
| chr9 | 130700674 | 130700891 | 0.000339757 | −22.30865801 | Hypo-methylated | PCa\|NT DMRs |
| chr9 | 130832419 | 130832953 | 0.003448944 | −34.85714286 | Hypo-methylated | PCa\|NT DMRs |
| chr9 | 131170888 | 131171294 | 0.00179283 | 32.25238095 | Hyper-methylated | PCa\|NT DMRs |
| chr9 | 131175224 | 131176304 | 0.001171269 | −21.94978355 | Hypo-methylated | PCa\|NT DMRs |
| chr9 | 131386788 | 131387123 | 0.004037929 | −42.10204082 | Hypo-methylated | PCa\|NT DMRs |
| chr9 | 131465280 | 131466577 | 1.38E−10 | 30.27512315 | Hyper-methylated | PCa\|NT DMRs |
| chr9 | 131683132 | 131683296 | 0.001969641 | −47.11904762 | Hypo-methylated | PCa\|NT DMRs |
| chr9 | 131900965 | 131901396 | 0.001181579 | −47.69795918 | Hypo-methylated | PCa\|NT DMRs |
| chr9 | 131924511 | 131924927 | 0.005513661 | 25.91666667 | Hyper-methylated | PCa\|NT DMRs |
| chr9 | 132033427 | 132033681 | 0.007560755 | 35.23214286 | Hyper-methylated | PCa\|NT DMRs |
| chr9 | 132044733 | 132045773 | 0.001326135 | 23.88291317 | Hyper-methylated | PCa\|NT DMRs |
| chr9 | 132103515 | 132103775 | 0.002575989 | 24.62857143 | Hyper-methylated | PCa\|NT DMRs |
| chr9 | 132105090 | 132110094 | 5.39E−07 | 29.54326241 | Hyper-methylated | PCa\|NT DMRs |
| chr9 | 132113450 | 132113607 | 0.001344447 | 30.38214286 | Hyper-methylated | PCa\|NT DMRs |
| chr9 | 132114691 | 132115040 | 2.88E−05 | 35.56302521 | Hyper-methylated | PCa\|NT DMRs |
| chr9 | 132140620 | 132140820 | 0.007560755 | −31.81547619 | Hypo-methylated | PCa\|NT DMRs |
| chr9 | 132146225 | 132146375 | 1.17E−06 | 32.84461153 | Hyper-methylated | PCa\|NT DMRs |
| chr9 | 132200296 | 132201118 | 5.51E−08 | 33.07942857 | Hyper-methylated | PCa\|NT DMRs |
| chr9 | 132221713 | 132221936 | 6.94E−06 | 42.39047619 | Hyper-methylated | PCa\|NT DMRs |
| chr9 | 132339296 | 132339428 | 0.001139334 | 26.54761905 | Hyper-methylated | PCa\|NT DMRs |
| chr9 | 132360185 | 132360352 | 0.007959104 | 23.53741497 | Hyper-methylated | PCa\|NT DMRs |
| chr9 | 132513968 | 132515259 | 2.12E−10 | −33.7258658 | Hypo-methylated | PCa\|NT DMRs |
| chr9 | 132539132 | 132540261 | 0.008109221 | 30.72142857 | Hyper-methylated | PCa\|NT DMRs |
| chr9 | 132547203 | 132547573 | 0.005968871 | −40.88571429 | Hypo-methylated | PCa\|NT DMRs |
| chr9 | 132573788 | 132574027 | 0.008482405 | −53.66761905 | Hypo-methylated | PCa\|NT DMRs |
| chr9 | 132625698 | 132628923 | 0.000205174 | 28.90022676 | Hyper-methylated | PCa\|NT DMRs |
| chr9 | 132630840 | 132631703 | 1.90E−05 | 26.22574956 | Hyper-methylated | PCa\|NT DMRs |
| chr9 | 132806203 | 132806281 | 0.001969641 | −25.07142857 | Hypo-methylated | PCa\|NT DMRs |
| chr9 | 132902616 | 132914133 | 0.004668266 | 24.87012987 | Hyper-methylated | PCa\|NT DMRs |
| chr9 | 132935542 | 132935819 | 6.83E−06 | 20.69691877 | Hyper-methylated | PCa\|NT DMRs |
| chr9 | 133320532 | 133320970 | 6.38E−09 | 24.44704185 | Hyper-methylated | PCa\|NT DMRs |
| chr9 | 133335225 | 133335709 | 0.00179283 | −32.6547619 | Hypo-methylated | PCa\|NT DMRs |
| chr9 | 133347391 | 133348397 | 9.19E−06 | 21.0499232 | Hyper-methylated | PCa\|NT DMRs |
| chr9 | 133534738 | 133534957 | 3.40E−07 | 21.96942356 | Hyper-methylated | PCa\|NT DMRs |
| chr9 | 133759698 | 133760095 | 0.000118421 | −27.97619048 | Hypo-methylated | PCa\|NT DMRs |
| chr9 | 133794267 | 133794512 | 0.000494377 | −23.23469388 | Hypo-methylated | PCa\|NT DMRs |
| chr9 | 133873921 | 133874195 | 0.001969641 | −29.88571429 | Hypo-methylated | PCa\|NT DMRs |
| chr9 | 133887793 | 133887962 | 0.00203766 | 20.27529762 | Hyper-methylated | PCa\|NT DMRs |
| chr9 | 133888184 | 133888470 | 0.002225326 | −20.83435374 | Hypo-methylated | PCa\|NT DMRs |
| chr9 | 133927945 | 133936600 | 0.000255766 | −27.40301587 | Hypo-methylated | PCa\|NT DMRs |
| chr9 | 133943638 | 133945604 | 0.000145094 | −31 | Hypo-methylated | PCa\|NT DMRs |
| chr9 | 134127729 | 134134288 | 9.89E−08 | 39.82989418 | Hyper-methylated | PCa\|NT DMRs |
| chr9 | 134140802 | 134151912 | 2.39E−12 | 39.86805556 | Hyper-methylated | PCa\|NT DMRs |
| chr9 | 134210073 | 134210206 | 0.002097193 | 28.47278912 | Hyper-methylated | PCa\|NT DMRs |
| chr9 | 134212146 | 134220286 | 0.004047992 | −27.46428571 | Hypo-methylated | PCa\|NT DMRs |
| chr9 | 134297287 | 134299109 | 1.46E−06 | −49.08174603 | Hypo-methylated | PCa\|NT DMRs |
| chr9 | 134473572 | 134478164 | 0.001756926 | 30.19220779 | Hyper-methylated | PCa\|NT DMRs |
| chr9 | 134604995 | 134609201 | 0.000615345 | 40.07509158 | Hyper-methylated | PCa\|NT DMRs |
| chr9 | 134630119 | 134630474 | 0.000205174 | −55.63095238 | Hypo-methylated | PCa\|NT DMRs |
| chr9 | 134959086 | 135007487 | 0.0019106 | −34.88690476 | Hypo-methylated | PCa\|NT DMRs |
| chr9 | 135083747 | 135083907 | 0.001969641 | 36.10714286 | Hyper-methylated | PCa\|NT DMRs |
| chr9 | 135114175 | 135116391 | 0.001579579 | 23.73959184 | Hyper-methylated | PCa\|NT DMRs |
| chr9 | 135300108 | 135302910 | 0.000140867 | 21.50151515 | Hyper-methylated | PCa\|NT DMRs |
| chr9 | 135362072 | 135362111 | 0.004095673 | −24.23809524 | Hypo-methylated | PCa\|NT DMRs |
| chr9 | 135645997 | 135646062 | 1.97E−05 | 37.24285714 | Hyper-methylated | PCa\|NT DMRs |
| chr9 | 135878361 | 135879598 | 7.18E−06 | 36.31142857 | Hyper-methylated | PCa\|NT DMRs |
| chr9 | 135926228 | 135926392 | 0.000205174 | −44.60714286 | Hypo-methylated | PCa\|NT DMRs |
| chr9 | 135987563 | 135990706 | 0.000154566 | 26.72619048 | Hyper-methylated | PCa\|NT DMRs |
| chr9 | 135994524 | 135995266 | 1.44E−08 | −32.85839599 | Hypo-methylated | PCa\|NT DMRs |
| chr9 | 136004538 | 136004699 | 0.001969641 | −27.88888889 | Hypo-methylated | PCa\|NT DMRs |
| chr9 | 136005295 | 136006562 | 0.005513661 | 25.21130952 | Hyper-methylated | PCa\|NT DMRs |
| chr9 | 136038535 | 136038637 | 0.00641359 | −22.47619048 | Hypo-methylated | PCa\|NT DMRs |
| chr9 | 136055928 | 136060069 | 0.005968871 | −41.65714286 | Hypo-methylated | PCa\|NT DMRs |
| chr9 | 136081567 | 136082059 | 0.000787744 | −30.64285714 | Hypo-methylated | PCa\|NT DMRs |
| chr9 | 136181528 | 136181805 | 1.52E−07 | −22.21258503 | Hypo-methylated | PCa\|NT DMRs |
| chr9 | 136182643 | 136190423 | 0.000564835 | −23.85934066 | Hypo-methylated | PCa\|NT DMRs |
| chr9 | 136223912 | 136225151 | 0.002142733 | −33.26825397 | Hypo-methylated | PCa\|NT DMRs |
| chr9 | 136261602 | 136262359 | 0.005855202 | −24.41369048 | Hypo-methylated | PCa\|NT DMRs |
| chr9 | 136325914 | 136325984 | 0.002097193 | −36.05442177 | Hypo-methylated | PCa\|NT DMRs |
| chr9 | 136340066 | 136341926 | 0.003736413 | 20.17800454 | Hyper-methylated | PCa\|NT DMRs |
| chr9 | 136350307 | 136350525 | 0.00064571 | −51.31496599 | Hypo-methylated | PCa\|NT DMRs |
| chr9 | 136355192 | 136357334 | 8.48E−06 | −27.99440994 | Hypo-methylated | PCa\|NT DMRs |
| chr9 | 136390944 | 136391008 | 0.001969641 | −48.86111111 | Hypo-methylated | PCa\|NT DMRs |
| chr9 | 136444128 | 136444370 | 3.65E−07 | 40.43106576 | Hyper-methylated | PCa\|NT DMRs |
| chr9 | 136454214 | 136460456 | 0.000131723 | −23.82401656 | Hypo-methylated | PCa\|NT DMRs |
| chr9 | 136487112 | 136494527 | 1.97E−05 | −28.25857143 | Hypo-methylated | PCa\|NT DMRs |
| chr9 | 136531040 | 136531768 | 0.001181579 | −21.26530612 | Hypo-methylated | PCa\|NT DMRs |
| chr9 | 136566356 | 136567309 | 2.64E−09 | 21.59447005 | Hyper-methylated | PCa\|NT DMRs |
| chr9 | 136567547 | 136567774 | 0.000117699 | 36.80519481 | Hyper-methylated | PCa\|NT DMRs |
| chr9 | 136650408 | 136656275 | 0.000376085 | 22.28119048 | Hyper-methylated | PCa\|NT DMRs |
| chr9 | 136720738 | 136721287 | 0.00576331 | 45.48214286 | Hyper-methylated | PCa\|NT DMRs |
| chr9 | 136727388 | 136728083 | 0.001992179 | 48.64761905 | Hyper-methylated | PCa\|NT DMRs |
| chr9 | 136728421 | 136729151 | 0.002373155 | −30.3042328 | Hypo-methylated | PCa\|NT DMRs |
| chr9 | 136780052 | 136780592 | 0.001775064 | 23.26785714 | Hyper-methylated | PCa\|NT DMRs |
| chr9 | 136797223 | 136797607 | 0.000205174 | −48.87698413 | Hypo-methylated | PCa\|NT DMRs |
| chr9 | 137184929 | 137186387 | 0.00020562 | 20.84586466 | Hyper-methylated | PCa\|NT DMRs |
| chr9 | 137230496 | 137236620 | 2.09E−08 | 33.44629014 | Hyper-methylated | PCa\|NT DMRs |
| chr9 | 137259594 | 137259966 | 1.42E−05 | −46.93401361 | Hypo-methylated | PCa\|NT DMRs |
| chr9 | 137272603 | 137274647 | 6.32E−07 | 34.08924731 | Hyper-methylated | PCa\|NT DMRs |
| chr9 | 137277731 | 137278107 | 1.42E−05 | 41.66496599 | Hyper-methylated | PCa\|NT DMRs |
| chr9 | 137278588 | 137279595 | 9.76E−07 | −44.1032967 | Hypo-methylated | PCa\|NT DMRs |
| chr9 | 137287071 | 137289437 | 2.80E−10 | 48.47380952 | Hyper-methylated | PCa\|NT DMRs |
| chr9 | 137304302 | 137304489 | 0.000575877 | 35.81601732 | Hyper-methylated | PCa\|NT DMRs |
| chr9 | 137324570 | 137324813 | 0.000191452 | 20.83587302 | Hyper-methylated | PCa\|NT DMRs |
| chr9 | 137347896 | 137348055 | 6.93E−09 | 28.77244898 | Hyper-methylated | PCa\|NT DMRs |
| chr9 | 137662668 | 137663197 | 0.000137816 | 30.58333333 | Hyper-methylated | PCa\|NT DMRs |
| chr9 | 137734281 | 137750605 | 8.64E−09 | −20.30021645 | Hypo-methylated | PCa\|NT DMRs |
| chr9 | 137968125 | 137968276 | 0.001111028 | 25.31746032 | Hyper-methylated | PCa\|NT DMRs |
| chr9 | 137980515 | 137981306 | 0.000149331 | 26.09029982 | Hyper-methylated | PCa\|NT DMRs |
| chr9 | 137990397 | 137990588 | 0.000205174 | 38.26785714 | Hyper-methylated | PCa\|NT DMRs |
| chr9 | 138047574 | 138067125 | 8.83E−19 | −28.5424812 | Hypo-methylated | PCa\|NT DMRs |
| chr9 | 138076200 | 138076479 | 6.42E−05 | −29.15555556 | Hypo-methylated | PCa\|NT DMRs |
| chr9 | 138109947 | 138110400 | 0.000783791 | −30.15238095 | Hypo-methylated | PCa\|NT DMRs |
| chr9 | 138135823 | 138136098 | 6.93E−06 | 30.00952381 | Hyper-methylated | PCa\|NT DMRs |
| chr9 | 138136674 | 138136896 | 0.005968871 | −46.73809524 | Hypo-methylated | PCa\|NT DMRs |
| chr9 | 138149841 | 138154779 | 1.46E−06 | −25.71496599 | Hypo-methylated | PCa\|NT DMRs |
| chr9 | 138193989 | 138200176 | 7.47E−07 | −25.56371882 | Hypo-methylated | PCa\|NT DMRs |
| chr9 | 138285343 | 138285756 | 0.000205174 | −47.47857143 | Hypo-methylated | PCa\|NT DMRs |
| chr9 | 138591535 | 138592709 | 7.74E−05 | −33.48511905 | Hypo-methylated | PCa\|NT DMRs |
| chr9 | 138672689 | 138675058 | 9.71E−09 | −23.65463659 | Hypo-methylated | PCa\|NT DMRs |
| chr9 | 138680915 | 138681142 | 0.000321712 | −21.3707483 | Hypo-methylated | PCa\|NT DMRs |
| chr9 | 138850454 | 138850548 | 0.005968871 | 60.61428571 | Hyper-methylated | PCa\|NT DMRs |
| chr9 | 138870677 | 138871546 | 8.49E−07 | 32.17063492 | Hyper-methylated | PCa\|NT DMRs |
| chr9 | 138874749 | 138876880 | 0.001430294 | −21.91957672 | Hypo-methylated | PCa\|NT DMRs |
| chr9 | 138976254 | 138976721 | 0.001201396 | 24.96507937 | Hyper-methylated | PCa\|NT DMRs |
| chr9 | 138980414 | 138982640 | 0.004676045 | 33.7015873 | Hyper-methylated | PCa\|NT DMRs |
| chr9 | 138985226 | 138985317 | 0.003622801 | −47.5952381 | Hypo-methylated | PCa\|NT DMRs |
| chr9 | 138990658 | 138991054 | 0.000260936 | 23.28571429 | Hyper-methylated | PCa\|NT DMRs |
| chr9 | 138991834 | 138992893 | 0.00101366 | −24.83619048 | Hypo-methylated | PCa\|NT DMRs |
| chr9 | 139001785 | 139003040 | 7.39E−07 | 40.95361552 | Hyper-methylated | PCa\|NT DMRs |
| chr9 | 139025711 | 139026245 | 2.40E−06 | 31.03361345 | Hyper-methylated | PCa\|NT DMRs |
| chr9 | 139065586 | 139069481 | 6.73E−05 | −27.638322 | Hypo-methylated | PCa\|NT DMRs |
| chr9 | 139080397 | 139080622 | 0.005968871 | −32.36666667 | Hypo-methylated | PCa\|NT DMRs |
| chr9 | 139121194 | 139121849 | 0.000176046 | −37.15151515 | Hypo-methylated | PCa\|NT DMRs |
| chr9 | 139146348 | 139148504 | 5.99E−05 | −29.32051282 | Hypo-methylated | PCa\|NT DMRs |
| chr9 | 139220532 | 139221347 | 4.40E−07 | 36.41519274 | Hyper-methylated | PCa\|NT DMRs |
| chr9 | 139237010 | 139237159 | 0.000190021 | 28.12987013 | Hyper-methylated | PCa\|NT DMRs |
| chr9 | 139239762 | 139240697 | 2.65E−09 | −38.50571429 | Hypo-methylated | PCa\|NT DMRs |
| chr9 | 139247030 | 139250363 | 7.82E−07 | −21.89395314 | Hypo-methylated | PCa\|NT DMRs |
| chr9 | 139260361 | 139260748 | 0.005513661 | 39.81071429 | Hyper-methylated | PCa\|NT DMRs |
| chr9 | 139267318 | 139268274 | 0.002585277 | 37.11309524 | Hyper-methylated | PCa\|NT DMRs |
| chr9 | 139271249 | 139272186 | 0.000632596 | 28.57359307 | Hyper-methylated | PCa\|NT DMRs |
| chr9 | 139371999 | 139375066 | 0.009407512 | −29.82539683 | Hypo-methylated | PCa\|NT DMRs |
| chr9 | 139378639 | 139379167 | 2.75E−05 | −23.76917989 | Hypo-methylated | PCa\|NT DMRs |
| chr9 | 139416961 | 139417517 | 2.04E−06 | 29.54675325 | Hyper-methylated | PCa\|NT DMRs |
| chr9 | 139426412 | 139435077 | 1.31E−13 | 35.21939605 | Hyper-methylated | PCa\|NT DMRs |
| chr9 | 139472661 | 139473954 | 0.000227038 | 30.4534632 | Hyper-methylated | PCa\|NT DMRs |
| chr9 | 139482489 | 139483401 | 8.87E−06 | 34.74285714 | Hyper-methylated | PCa\|NT DMRs |
| chr9 | 139494430 | 139494944 | 0.005375157 | 25.3244898 | Hyper-methylated | PCa\|NT DMRs |
| chr9 | 139498484 | 139498705 | 1.06E−07 | 43.49773243 | Hyper-methylated | PCa\|NT DMRs |
| chr9 | 139506187 | 139506436 | 1.69E−06 | −45.6697479 | Hypo-methylated | PCa\|NT DMRs |
| chr9 | 139511930 | 139515432 | 4.56E−16 | 32.02928827 | Hyper-methylated | PCa\|NT DMRs |
| chr9 | 139515605 | 139518540 | 0.000149331 | 22.59159068 | Hyper-methylated | PCa\|NT DMRs |
| chr9 | 139522237 | 139523438 | 3.39E−08 | 32.51291209 | Hyper-methylated | PCa\|NT DMRs |
| chr9 | 139539576 | 139546145 | 2.01E−17 | 35.12148148 | Hyper-methylated | PCa\|NT DMRs |
| chr9 | 139578490 | 139581203 | 3.00E−07 | 22.94533333 | Hyper-methylated | PCa\|NT DMRs |
| chr9 | 139587793 | 139589213 | 0.001419801 | −20.60952381 | Hypo-methylated | PCa\|NT DMRs |
| chr9 | 139589454 | 139589673 | 0.000205174 | −42.07142857 | Hypo-methylated | PCa\|NT DMRs |
| chr9 | 139607683 | 139610652 | 6.51E−06 | 38.81096681 | Hyper-methylated | PCa\|NT DMRs |
| chr9 | 139611890 | 139613872 | 4.11E−05 | 31.21025641 | Hyper-methylated | PCa\|NT DMRs |
| chr9 | 139654998 | 139656264 | 0.00023371 | −30.1 | Hypo-methylated | PCa\|NT DMRs |
| chr9 | 139725254 | 139725728 | 6.42E−05 | 37.17724868 | Hyper-methylated | PCa\|NT DMRs |
| chr9 | 139734936 | 139735683 | 2.78E−05 | −30.83110119 | Hypo-methylated | PCa\|NT DMRs |
| chr9 | 139796950 | 139797247 | 0.000117699 | −29.46969697 | Hypo-methylated | PCa\|NT DMRs |
| chr9 | 139819960 | 139820197 | 0.005685503 | −30.93650794 | Hypo-methylated | PCa\|NT DMRs |
| chr9 | 139837842 | 139837963 | 0.001969641 | −54.28571429 | Hypo-methylated | PCa\|NT DMRs |
| chr9 | 139841454 | 139842379 | 6.34E−06 | −29.8422619 | Hypo-methylated | PCa\|NT DMRs |
| chr9 | 139856054 | 139856311 | 0.00068166 | −27.26488095 | Hypo-methylated | PCa\|NT DMRs |
| chr9 | 139868939 | 139871011 | 0.00023371 | 37.21296296 | Hyper-methylated | PCa\|NT DMRs |
| chr9 | 139889122 | 139892886 | 1.83E−06 | 26.30505345 | Hyper-methylated | PCa\|NT DMRs |
| chr9 | 139925092 | 139929160 | 4.37E−08 | 25.04686319 | Hyper-methylated | PCa\|NT DMRs |
| chr9 | 139952153 | 139952825 | 0.000145094 | −53.35436508 | Hypo-methylated | PCa\|NT DMRs |
| chr9 | 140024941 | 140025119 | 0.001055672 | 24.82653061 | Hyper-methylated | PCa\|NT DMRs |
| chr9 | 140051178 | 140051233 | 1.95E−06 | 23 | Hyper-methylated | PCa\|NT DMRs |
| chr9 | 140111534 | 140114761 | 1.56E−11 | −40.91977226 | Hypo-methylated | PCa\|NT DMRs |
| chr9 | 140127033 | 140127499 | 1.35E−06 | −22.49689441 | Hypo-methylated | PCa\|NT DMRs |
| chr9 | 140171190 | 140172989 | 4.08E−14 | 38.32194211 | Hyper-methylated | PCa\|NT DMRs |
| chr9 | 140187609 | 140187811 | 0.000260936 | −38.10952381 | Hypo-methylated | PCa\|NT DMRs |
| chr9 | 140214591 | 140215570 | 0.00358897 | 28.87968254 | Hyper-methylated | PCa\|NT DMRs |
| chr9 | 140285007 | 140285942 | 8.74E−05 | −33.60910973 | Hypo-methylated | PCa\|NT DMRs |
| chr9 | 140301982 | 140302294 | 0.00355142 | −25.01575092 | Hypo-methylated | PCa\|NT DMRs |
| chr9 | 140305351 | 140307130 | 3.37E−06 | 27.82751323 | Hyper-methylated | PCa\|NT DMRs |
| chr9 | 140311098 | 140312521 | 1.16E−21 | 31.19112782 | Hyper-methylated | PCa\|NT DMRs |
| chr9 | 140329182 | 140329356 | 0.000205174 | 47.71428571 | Hyper-methylated | PCa\|NT DMRs |
| chr9 | 140330432 | 140334082 | 3.01E−09 | −29.49047619 | Hypo-methylated | PCa\|NT DMRs |
| chr9 | 140341458 | 140341607 | 0.005968871 | 44.68095238 | Hyper-methylated | PCa\|NT DMRs |
| chr9 | 140459598 | 140462140 | 0.001969641 | 43.12857143 | Hyper-methylated | PCa\|NT DMRs |
| chr9 | 140472268 | 140472540 | 0.007959104 | −24.39795918 | Hypo-methylated | PCa\|NT DMRs |
| chr9 | 140485693 | 140488177 | 0.00358897 | −35.22285714 | Hypo-methylated | PCa\|NT DMRs |
| chr9 | 140509383 | 140510053 | 0.001181579 | −45.42857143 | Hypo-methylated | PCa\|NT DMRs |
| chr9 | 140651601 | 140651808 | 0.001969641 | −57.87755102 | Hypo-methylated | PCa\|NT DMRs |
| chr9 | 140959802 | 140963403 | 0.001241245 | −23.53703704 | Hypo-methylated | PCa\|NT DMRs |
| chr9 | 141013804 | 141015318 | 3.29E−10 | −21.13191763 | Hypo-methylated | PCa\|NT DMRs |
| chr10 | 556444 | 556706 | 0.000205174 | −26.30357143 | Hypo-methylated | PCa\|NT DMRs |
| chr10 | 743193 | 743313 | 5.84E−05 | 21.98809524 | Hyper-methylated | PCa\|NT DMRs |
| chr10 | 835645 | 836435 | 0.002595994 | −25.62275132 | Hypo-methylated | PCa\|NT DMRs |
| chr10 | 1155970 | 1156826 | 4.68E−05 | −24.1712522 | Hypo-methylated | PCa\|NT DMRs |
| chr10 | 1449800 | 1449999 | 0.007959104 | 22.89795918 | Hyper-methylated | PCa\|NT DMRs |
| chr10 | 2543994 | 2544058 | 0.00023371 | 28.58714286 | Hyper-methylated | PCa\|NT DMRs |
| chr10 | 3027998 | 3028236 | 0.005968871 | −23.24761905 | Hypo-methylated | PCa\|NT DMRs |
| chr10 | 3100042 | 3109394 | 1.25E−08 | 27.75649936 | Hyper-methylated | PCa\|NT DMRs |
| chr10 | 3138410 | 3138703 | 1.92E−05 | 46.87058824 | Hyper-methylated | PCa\|NT DMRs |
| chr10 | 3160926 | 3161297 | 1.31E−06 | −26.71693122 | Hypo-methylated | PCa\|NT DMRs |
| chr10 | 3234515 | 3237346 | 0.003871035 | −21.55357143 | Hypo-methylated | PCa\|NT DMRs |
| chr10 | 3302454 | 3302715 | 0.000354065 | −47.35291005 | Hypo-methylated | PCa\|NT DMRs |
| chr10 | 3502753 | 3512546 | 0.009106413 | −29.08253968 | Hypo-methylated | PCa\|NT DMRs |
| chr10 | 3594345 | 3596666 | 0.002529619 | 38.92307692 | Hyper-methylated | PCa\|NT DMRs |
| chr10 | 3763180 | 3788686 | 0.003290817 | 25.36071429 | Hyper-methylated | PCa\|NT DMRs |
| chr10 | 3791988 | 3792369 | 0.001969641 | 32.53673469 | Hyper-methylated | PCa\|NT DMRs |
| chr10 | 3823758 | 3823908 | 4.28E−07 | 33.53111111 | Hyper-methylated | PCa\|NT DMRs |
| chr10 | 3929019 | 3929291 | 0.00064571 | −55.57959184 | Hypo-methylated | PCa\|NT DMRs |
| chr10 | 5530284 | 5530781 | 0.002585277 | −20.92857143 | Hypo-methylated | PCa\|NT DMRs |
| chr10 | 5615986 | 5616349 | 2.05E−05 | −39.5969697 | Hypo-methylated | PCa\|NT DMRs |
| chr10 | 5621457 | 5621639 | 0.00068166 | −51.03869048 | Hypo-methylated | PCa\|NT DMRs |
| chr10 | 5731421 | 5734828 | 7.34E−05 | 35.61269841 | Hyper-methylated | PCa\|NT DMRs |
| chr10 | 6104182 | 6107976 | 0.002097193 | 23.30612245 | Hyper-methylated | PCa\|NT DMRs |
| chr10 | 6109062 | 6120302 | 0.000149331 | −27.17489177 | Hypo-methylated | PCa\|NT DMRs |
| chr10 | 6185717 | 6186026 | 5.19E−07 | 28.71648352 | Hyper-methylated | PCa\|NT DMRs |
| chr10 | 6187491 | 6187913 | 0.00188599 | 24.84126984 | Hyper-methylated | PCa\|NT DMRs |
| chr10 | 6218761 | 6219158 | 0.00064571 | 40.37414966 | Hyper-methylated | PCa\|NT DMRs |
| chr10 | 6246171 | 6246451 | 1.45E−05 | −20.64880952 | Hypo-methylated | PCa\|NT DMRs |
| chr10 | 6297892 | 6298058 | 0.001969641 | −40.26530612 | Hypo-methylated | PCa\|NT DMRs |
| chr10 | 6302269 | 6317678 | 0.008649834 | 25.42857143 | Hyper-methylated | PCa\|NT DMRs |
| chr10 | 6763532 | 6775012 | 0.00641359 | −48.3015873 | Hypo-methylated | PCa\|NT DMRs |
| chr10 | 8075716 | 8076637 | 5.33E−12 | 29.71863354 | Hyper-methylated | PCa\|NT DMRs |
| chr10 | 11472552 | 11475258 | 0.001344447 | −49.50892857 | Hypo-methylated | PCa\|NT DMRs |
| chr10 | 11504160 | 11504220 | 0.004095673 | 29.01984127 | Hyper-methylated | PCa\|NT DMRs |
| chr10 | 11682342 | 11684889 | 0.001969641 | 35.50714286 | Hyper-methylated | PCa\|NT DMRs |
| chr10 | 11737214 | 11739418 | 0.00576331 | −31.61011905 | Hypo-methylated | PCa\|NT DMRs |
| chr10 | 12380735 | 12390599 | 0.002373155 | −24.61587302 | Hypo-methylated | PCa\|NT DMRs |
| chr10 | 12390656 | 12391138 | 1.31E−13 | 20.55137845 | Hyper-methylated | PCa\|NT DMRs |
| chr10 | 12458651 | 12458983 | 0.000190021 | 28.30519481 | Hyper-methylated | PCa\|NT DMRs |
| chr10 | 12468406 | 12476786 | 8.49E−05 | −28.7568254 | Hypo-methylated | PCa\|NT DMRs |
| chr10 | 12676665 | 12676771 | 0.00064571 | 22.1877551 | Hyper-methylated | PCa\|NT DMRs |
| chr10 | 12948442 | 12951611 | 0.001583613 | −27.58614719 | Hypo-methylated | PCa\|NT DMRs |
| chr10 | 13043118 | 13043450 | 1.01E−06 | 23.97883598 | Hyper-methylated | PCa\|NT DMRs |
| chr10 | 13442612 | 13442783 | 0.005968871 | −32.4 | Hypo-methylated | PCa\|NT DMRs |
| chr10 | 14305211 | 14305371 | 0.005968871 | 43.22857143 | Hyper-methylated | PCa\|NT DMRs |
| chr10 | 15077227 | 15077528 | 0.000377317 | 30.11666667 | Hyper-methylated | PCa\|NT DMRs |
| chr10 | 21788694 | 21789702 | 2.69E−05 | 38.50412698 | Hyper-methylated | PCa\|NT DMRs |
| chr10 | 21798528 | 21804387 | 0.00015558 | 27.81165846 | Hyper-methylated | PCa\|NT DMRs |
| chr10 | 22660428 | 22660476 | 0.005968871 | −38.23333333 | Hypo-methylated | PCa\|NT DMRs |
| chr10 | 22760891 | 22766904 | 8.13E−20 | 34.17026307 | Hyper-methylated | PCa\|NT DMRs |
| chr10 | 23460647 | 23461376 | 0.001610287 | 27.44960317 | Hyper-methylated | PCa\|NT DMRs |
| chr10 | 23551094 | 23631462 | 4.82E−05 | −23.72071429 | Hypo-methylated | PCa\|NT DMRs |
| chr10 | 27234975 | 27235299 | 0.001076343 | −28.46753247 | Hypo-methylated | PCa\|NT DMRs |
| chr10 | 27519447 | 27527854 | 5.94E−06 | −31.2012987 | Hypo-methylated | PCa\|NT DMRs |
| chr10 | 27702649 | 27702849 | 5.84E−05 | 26.16071429 | Hyper-methylated | PCa\|NT DMRs |
| chr10 | 27978739 | 27978860 | 0.00064571 | −53.15646259 | Hypo-methylated | PCa\|NT DMRs |
| chr10 | 28030428 | 28030952 | 4.32E−05 | 45.58974359 | Hyper-methylated | PCa\|NT DMRs |
| chr10 | 28965390 | 28965937 | 1.65E−06 | 22.6366843 | Hyper-methylated | PCa\|NT DMRs |
| chr10 | 29201275 | 29214362 | 0.00179283 | 26.78253968 | Hyper-methylated | PCa\|NT DMRs |
| chr10 | 29228804 | 29265453 | 0.006589535 | −30.21428571 | Hypo-methylated | PCa\|NT DMRs |
| chr10 | 29666139 | 29667292 | 0.001480644 | −33.87714286 | Hypo-methylated | PCa\|NT DMRs |
| chr10 | 29771617 | 29771828 | 0.001969641 | 33.11904762 | Hyper-methylated | PCa\|NT DMRs |
| chr10 | 30452793 | 30452937 | 0.007338757 | 33.48214286 | Hyper-methylated | PCa\|NT DMRs |
| chr10 | 30765882 | 30765936 | 0.005968871 | 39.17142857 | Hyper-methylated | PCa\|NT DMRs |
| chr10 | 30950283 | 30973199 | 0.005572785 | −21.4959707 | Hypo-methylated | PCa\|NT DMRs |
| chr10 | 31875888 | 31876293 | 0.000205174 | 39.23571429 | Hyper-methylated | PCa\|NT DMRs |
| chr10 | 32039435 | 32046741 | 0.004417112 | −27.02040816 | Hypo-methylated | PCa\|NT DMRs |
| chr10 | 32514672 | 32522497 | 0.001969641 | −22.62698413 | Hypo-methylated | PCa\|NT DMRs |
| chr10 | 34827654 | 34827714 | 0.001969641 | −32.11904762 | Hypo-methylated | PCa\|NT DMRs |
| chr10 | 36188221 | 36188292 | 0.004095673 | −55.11904762 | Hypo-methylated | PCa\|NT DMRs |
| chr10 | 37448783 | 37483524 | 0.000205174 | −36.46428571 | Hypo-methylated | PCa\|NT DMRs |
| chr10 | 38300369 | 38300428 | 0.005513661 | −20.06122449 | Hypo-methylated | PCa\|NT DMRs |
| chr10 | 42490532 | 42644711 | 1.05E−09 | −23.64537037 | Hypo-methylated | PCa\|NT DMRs |
| chr10 | 42676531 | 42730315 | 0.002097193 | −27.33605442 | Hypo-methylated | PCa\|NT DMRs |
| chr10 | 43366821 | 43367062 | 0.001969641 | 27.66666667 | Hyper-methylated | PCa\|NT DMRs |
| chr10 | 43600358 | 43600734 | 8.41E−14 | 38.53117914 | Hyper-methylated | PCa\|NT DMRs |
| chr10 | 43846514 | 43846706 | 2.50E−05 | 25.31150794 | Hyper-methylated | PCa\|NT DMRs |
| chr10 | 43912393 | 43913156 | 0.003871035 | 36.33253968 | Hyper-methylated | PCa\|NT DMRs |
| chr10 | 44787939 | 44806375 | 0.00044067 | 26.27819549 | Hyper-methylated | PCa\|NT DMRs |
| chr10 | 45323025 | 45324261 | 3.20E−05 | −33.77380952 | Hypo-methylated | PCa\|NT DMRs |
| chr10 | 45741465 | 45745225 | 0.005968871 | −26.55384615 | Hypo-methylated | PCa\|NT DMRs |
| chr10 | 45755738 | 45755810 | 6.42E−05 | −33.32592593 | Hypo-methylated | PCa\|NT DMRs |
| chr10 | 46970108 | 46970377 | 4.00E−07 | 27.84432234 | Hyper-methylated | PCa\|NT DMRs |
| chr10 | 46993211 | 46993637 | 3.70E−06 | 33.44347826 | Hyper-methylated | PCa\|NT DMRs |
| chr10 | 47638123 | 47638209 | 0.001969641 | 37.34693878 | Hyper-methylated | PCa\|NT DMRs |
| chr10 | 48451549 | 48451641 | 0.005968871 | −52.43428571 | Hypo-methylated | PCa\|NT DMRs |
| chr10 | 48461111 | 48485587 | 6.65E−07 | −24.67094017 | Hypo-methylated | PCa\|NT DMRs |
| chr10 | 49459788 | 49459846 | 0.005968871 | −53.02857143 | Hypo-methylated | PCa\|NT DMRs |
| chr10 | 49664506 | 49668100 | 0.000552328 | 25.71164021 | Hyper-methylated | PCa\|NT DMRs |
| chr10 | 49866754 | 49891545 | 0.008234951 | −23.3452381 | Hypo-methylated | PCa\|NT DMRs |
| chr10 | 49892861 | 49892955 | 0.005968871 | 48.67619048 | Hyper-methylated | PCa\|NT DMRs |
| chr10 | 49899260 | 49916517 | 1.92E−05 | −26.32761905 | Hypo-methylated | PCa\|NT DMRs |
| chr10 | 50323793 | 50347113 | 2.26E−07 | −31.96 | Hypo-methylated | PCa\|NT DMRs |
| chr10 | 50373103 | 50374117 | 1.97E−05 | −33.28380952 | Hypo-methylated | PCa\|NT DMRs |
| chr10 | 50404163 | 50437652 | 4.28E−07 | −28.21580087 | Hypo-methylated | PCa\|NT DMRs |
| chr10 | 50650006 | 50650326 | 0.000909347 | 22.46230159 | Hyper-methylated | PCa\|NT DMRs |
| chr10 | 50833615 | 50872572 | 1.62E−06 | −25.29437229 | Hypo-methylated | PCa\|NT DMRs |
| chr10 | 50904521 | 50943452 | 1.19E−05 | −22.5047619 | Hypo-methylated | PCa\|NT DMRs |
| chr10 | 51784605 | 51784889 | 2.06E−05 | 29.28469388 | Hyper-methylated | PCa\|NT DMRs |
| chr10 | 52763120 | 52764080 | 0.002097193 | −26.16666667 | Hypo-methylated | PCa\|NT DMRs |
| chr10 | 52883339 | 53159272 | 5.94E−06 | −34.26363636 | Hypo-methylated | PCa\|NT DMRs |
| chr10 | 53355683 | 53393542 | 0.00064571 | −33.93061224 | Hypo-methylated | PCa\|NT DMRs |
| chr10 | 56935279 | 57316424 | 8.42E−15 | −27.06092215 | Hypo-methylated | PCa\|NT DMRs |
| chr10 | 57407046 | 57407383 | 0.001181579 | −26.57142857 | Hypo-methylated | PCa\|NT DMRs |
| chr10 | 57842848 | 57862541 | 0.005968871 | −26.17142857 | Hypo-methylated | PCa\|NT DMRs |
| chr10 | 58346328 | 58456612 | 0.000335271 | −25.15384615 | Hypo-methylated | PCa\|NT DMRs |
| chr10 | 58671006 | 58817079 | 3.36E−06 | −25.49068323 | Hypo-methylated | PCa\|NT DMRs |
| chr10 | 63627786 | 63627947 | 0.00064571 | 38.37959184 | Hyper-methylated | PCa\|NT DMRs |
| chr10 | 63808929 | 63809171 | 2.85E−05 | −42.74908425 | Hypo-methylated | PCa\|NT DMRs |
| chr10 | 64646645 | 64678424 | 0.00064571 | −30.61088435 | Hypo-methylated | PCa\|NT DMRs |
| chr10 | 66084754 | 66170204 | 2.13E−06 | −30.54738095 | Hypo-methylated | PCa\|NT DMRs |
| chr10 | 69609835 | 69610039 | 0.00603903 | −25.72857143 | Hypo-methylated | PCa\|NT DMRs |
| chr10 | 70321294 | 70327140 | 0.005685503 | 26.375 | Hyper-methylated | PCa\|NT DMRs |
| chr10 | 70846441 | 70861730 | 0.006207271 | 28.11507937 | Hyper-methylated | PCa\|NT DMRs |
| chr10 | 71122398 | 71135706 | 0.007560755 | −22.95343915 | Hypo-methylated | PCa\|NT DMRs |
| chr10 | 71214813 | 71232517 | 0.007560755 | 21.20535714 | Hyper-methylated | PCa\|NT DMRs |
| chr10 | 71422000 | 71422301 | 0.000974795 | −32.92559524 | Hypo-methylated | PCa\|NT DMRs |
| chr10 | 71475998 | 71476234 | 0.003448944 | −22.30272109 | Hypo-methylated | PCa\|NT DMRs |
| chr10 | 71577803 | 71583584 | 0.003871035 | −20.80357143 | Hypo-methylated | PCa\|NT DMRs |
| chr10 | 71596002 | 71599682 | 0.006589535 | 30.71190476 | Hyper-methylated | PCa\|NT DMRs |
| chr10 | 71721702 | 71721874 | 0.005968871 | 47.84761905 | Hyper-methylated | PCa\|NT DMRs |
| chr10 | 72043396 | 72043785 | 9.29E−07 | 20.50584416 | Hyper-methylated | PCa\|NT DMRs |
| chr10 | 72218877 | 72220077 | 0.003764982 | 26.40793651 | Hyper-methylated | PCa\|NT DMRs |
| chr10 | 72253815 | 72260799 | 1.71E−06 | 32.81809524 | Hyper-methylated | PCa\|NT DMRs |
| chr10 | 72325278 | 72325450 | 0.005968871 | 42.99428571 | Hyper-methylated | PCa\|NT DMRs |
| chr10 | 72384433 | 72390950 | 0.00046742 | −20.81818182 | Hypo-methylated | PCa\|NT DMRs |
| chr10 | 72898069 | 72898339 | 0.000117699 | −47.93506494 | Hypo-methylated | PCa\|NT DMRs |
| chr10 | 73062374 | 73062852 | 0.007560755 | −28.07738095 | Hypo-methylated | PCa\|NT DMRs |
| chr10 | 73073033 | 73077029 | 0.000205174 | −51.49107143 | Hypo-methylated | PCa\|NT DMRs |
| chr10 | 73155765 | 73156525 | 4.08E−06 | 29.7020592 | Hyper-methylated | PCa\|NT DMRs |
| chr10 | 73398524 | 73398901 | 3.05E−06 | 37.21978022 | Hyper-methylated | PCa\|NT DMRs |
| chr10 | 73426527 | 73426831 | 0.001181579 | 36.34693878 | Hyper-methylated | PCa\|NT DMRs |
| chr10 | 73472344 | 73475785 | 0.003702731 | 29.14285714 | Hyper-methylated | PCa\|NT DMRs |
| chr10 | 73484911 | 73490384 | 0.000946908 | 35.23116883 | Hyper-methylated | PCa\|NT DMRs |
| chr10 | 73494321 | 73506192 | 6.25E−05 | 25.13646259 | Hyper-methylated | PCa\|NT DMRs |
| chr10 | 73557712 | 73558030 | 0.00064571 | 43.58503401 | Hyper-methylated | PCa\|NT DMRs |
| chr10 | 73723435 | 73723843 | 6.33E−05 | 26.6884058 | Hyper-methylated | PCa\|NT DMRs |
| chr10 | 73725083 | 73725232 | 0.00064571 | 34.73469388 | Hyper-methylated | PCa\|NT DMRs |
| chr10 | 73729855 | 73730385 | 0.002562638 | −40.49404762 | Hypo-methylated | PCa\|NT DMRs |
| chr10 | 73844109 | 73848209 | 3.82E−16 | 21.18701707 | Hyper-methylated | PCa\|NT DMRs |
| chr10 | 74020487 | 74020668 | 0.000377317 | 29.56428571 | Hyper-methylated | PCa\|NT DMRs |
| chr10 | 74035652 | 74036091 | 0.005968871 | −42.87142857 | Hypo-methylated | PCa\|NT DMRs |
| chr10 | 74088723 | 74095793 | 0.000455762 | 26.15014881 | Hyper-methylated | PCa\|NT DMRs |
| chr10 | 74928939 | 74938815 | 0.002097193 | −29.42857143 | Hypo-methylated | PCa\|NT DMRs |
| chr10 | 75475351 | 75475426 | 0.005968871 | −48.03904762 | Hypo-methylated | PCa\|NT DMRs |
| chr10 | 75579349 | 75579764 | 8.29E−07 | −49.02380952 | Hypo-methylated | PCa\|NT DMRs |
| chr10 | 76502199 | 76503649 | 0.001789867 | 33.54497354 | Hyper-methylated | PCa\|NT DMRs |
| chr10 | 76803562 | 76812891 | 3.67E−06 | 31.79333333 | Hyper-methylated | PCa\|NT DMRs |
| chr10 | 79396120 | 79396710 | 2.92E−06 | 20.09897959 | Hyper-methylated | PCa\|NT DMRs |
| chr10 | 79469897 | 79471025 | 0.003362655 | 23.73296703 | Hyper-methylated | PCa\|NT DMRs |
| chr10 | 79637737 | 79637988 | 0.005968871 | 37.8 | Hyper-methylated | PCa\|NT DMRs |
| chr10 | 80030398 | 80037953 | 0.006225823 | −32.08138528 | Hypo-methylated | PCa\|NT DMRs |
| chr10 | 80891971 | 80911461 | 1.75E−06 | 24.40854978 | Hyper-methylated | PCa\|NT DMRs |
| chr10 | 80941264 | 80947304 | 0.000191452 | 27.64952381 | Hyper-methylated | PCa\|NT DMRs |
| chr10 | 80990136 | 80990405 | 0.001789867 | 32.39100529 | Hyper-methylated | PCa\|NT DMRs |
| chr10 | 81053182 | 81058392 | 0.002400984 | 26.10888889 | Hyper-methylated | PCa\|NT DMRs |
| chr10 | 81074936 | 81075242 | 0.000159287 | −33.14285714 | Hypo-methylated | PCa\|NT DMRs |
| chr10 | 81163196 | 81175656 | 1.13E−05 | 41.73113553 | Hyper-methylated | PCa\|NT DMRs |
| chr10 | 81206455 | 81212255 | 0.005968871 | −28.29375 | Hypo-methylated | PCa\|NT DMRs |
| chr10 | 81742882 | 81743215 | 1.72E−05 | 23.98401361 | Hyper-methylated | PCa\|NT DMRs |
| chr10 | 81928358 | 81928878 | 0.002437742 | −22.45661376 | Hypo-methylated | PCa\|NT DMRs |
| chr10 | 82269180 | 82269399 | 0.00064571 | −25.09387755 | Hypo-methylated | PCa\|NT DMRs |
| chr10 | 83683256 | 83910012 | 5.52E−05 | −27.94829932 | Hypo-methylated | PCa\|NT DMRs |
| chr10 | 85997015 | 85997267 | 5.95E−07 | 27.28571429 | Hyper-methylated | PCa\|NT DMRs |
| chr10 | 86004879 | 86014216 | 0.004553481 | −23.07460317 | Hypo-methylated | PCa\|NT DMRs |
| chr10 | 86335270 | 86444165 | 2.35E−10 | −25.32125436 | Hypo-methylated | PCa\|NT DMRs |
| chr10 | 86522263 | 86732049 | 2.07E−07 | −28.32326007 | Hypo-methylated | PCa\|NT DMRs |
| chr10 | 87967682 | 88010471 | 8.35E−08 | −29.10978121 | Hypo-methylated | PCa\|NT DMRs |
| chr10 | 88127296 | 88127532 | 4.24E−06 | 24.63909774 | Hyper-methylated | PCa\|NT DMRs |
| chr10 | 88294981 | 88295574 | 8.39E−13 | −24.65452848 | Hypo-methylated | PCa\|NT DMRs |
| chr10 | 88376246 | 88392096 | 4.75E−06 | 24.43581781 | Hyper-methylated | PCa\|NT DMRs |
| chr10 | 88728350 | 88728573 | 1.73E−09 | 22.20798771 | Hyper-methylated | PCa\|NT DMRs |
| chr10 | 88730081 | 88731649 | 5.37E−22 | 33.62130326 | Hyper-methylated | PCa\|NT DMRs |
| chr10 | 88976060 | 88976088 | 0.005968871 | 42.71428571 | Hyper-methylated | PCa\|NT DMRs |
| chr10 | 90686379 | 90712685 | 0.005425083 | −33.26785714 | Hypo-methylated | PCa\|NT DMRs |
| chr10 | 90966717 | 90966942 | 0.000480383 | 21.88359788 | Hyper-methylated | PCa\|NT DMRs |
| chr10 | 92616684 | 92616848 | 0.000377317 | 26.04166667 | Hyper-methylated | PCa\|NT DMRs |
| chr10 | 93504923 | 93505044 | 0.005968871 | −45.11428571 | Hypo-methylated | PCa\|NT DMRs |
| chr10 | 93999137 | 93999348 | 7.88E−06 | −37.73809524 | Hypo-methylated | PCa\|NT DMRs |
| chr10 | 94449893 | 94451993 | 1.31E−25 | 29.08099174 | Hyper-methylated | PCa\|NT DMRs |
| chr10 | 94834546 | 94835374 | 1.87E−05 | 25.39455782 | Hyper-methylated | PCa\|NT DMRs |
| chr10 | 95360289 | 95360614 | 0.001294067 | 24.01410935 | Hyper-methylated | PCa\|NT DMRs |
| chr10 | 95683692 | 95694163 | 0.003622801 | −20.97619048 | Hypo-methylated | PCa\|NT DMRs |
| chr10 | 98272234 | 98272754 | 5.37E−10 | 22.53243243 | Hyper-methylated | PCa\|NT DMRs |
| chr10 | 98405168 | 98446983 | 0.000205174 | 34.05619048 | Hyper-methylated | PCa\|NT DMRs |
| chr10 | 98760392 | 98760812 | 0.001480644 | −36.59047619 | Hypo-methylated | PCa\|NT DMRs |
| chr10 | 98913759 | 98923658 | 0.005968871 | −21.6047619 | Hypo-methylated | PCa\|NT DMRs |
| chr10 | 99125969 | 99126316 | 0.000443725 | 27.55119048 | Hyper-methylated | PCa\|NT DMRs |
| chr10 | 99664428 | 99665496 | 0.000286238 | 23.00915751 | Hyper-methylated | PCa\|NT DMRs |
| chr10 | 99790772 | 99798763 | 3.69E−08 | 25.2609127 | Hyper-methylated | PCa\|NT DMRs |
| chr10 | 100150666 | 100150938 | 0.001969641 | 36.14047619 | Hyper-methylated | PCa\|NT DMRs |
| chr10 | 100227729 | 100282177 | 0.004497523 | 21.29503968 | Hyper-methylated | PCa\|NT DMRs |
| chr10 | 101279686 | 101280310 | 0.000649993 | 32.31501832 | Hyper-methylated | PCa\|NT DMRs |
| chr10 | 101283053 | 101283302 | 0.000974795 | 25.26130952 | Hyper-methylated | PCa\|NT DMRs |
| chr10 | 101295496 | 101300263 | 4.44E−15 | 33.24034632 | Hyper-methylated | PCa\|NT DMRs |
| chr10 | 101875003 | 101875060 | 1.42E−05 | 30.29931973 | Hyper-methylated | PCa\|NT DMRs |
| chr10 | 102329603 | 102329989 | 0.000847246 | 22.58945578 | Hyper-methylated | PCa\|NT DMRs |
| chr10 | 102748904 | 102749079 | 0.001969641 | −45.88095238 | Hypo-methylated | PCa\|NT DMRs |
| chr10 | 102791787 | 102792473 | 5.05E−15 | 21.59593254 | Hyper-methylated | PCa\|NT DMRs |
| chr10 | 102807574 | 102808087 | 2.08E−06 | 24.36153846 | Hyper-methylated | PCa\|NT DMRs |
| chr10 | 103043851 | 103044324 | 2.53E−10 | 37.2264881 | Hyper-methylated | PCa\|NT DMRs |
| chr10 | 103053118 | 103070481 | 8.87E−05 | 27.51580952 | Hyper-methylated | PCa\|NT DMRs |
| chr10 | 103329251 | 103329472 | 0.000632596 | 20.51428571 | Hyper-methylated | PCa\|NT DMRs |
| chr10 | 103589048 | 103589516 | 0.000392715 | 32.24047619 | Hyper-methylated | PCa\|NT DMRs |
| chr10 | 103830984 | 103831047 | 0.000205174 | 40.5297619 | Hyper-methylated | PCa\|NT DMRs |
| chr10 | 104211167 | 104211389 | 0.005513661 | −23.83673469 | Hypo-methylated | PCa\|NT DMRs |
| chr10 | 104233380 | 104233458 | 0.000205174 | 26.71428571 | Hyper-methylated | PCa\|NT DMRs |
| chr10 | 104416887 | 104419465 | 5.58E−05 | −32.61851852 | Hypo-methylated | PCa\|NT DMRs |
| chr10 | 104433792 | 104434245 | 1.76E−06 | 47.08412698 | Hyper-methylated | PCa\|NT DMRs |
| chr10 | 104629681 | 104629912 | 0.00641359 | 31.00238095 | Hyper-methylated | PCa\|NT DMRs |
| chr10 | 104679287 | 104679334 | 0.002585277 | 21.75 | Hyper-methylated | PCa\|NT DMRs |
| chr10 | 105157285 | 105176631 | 2.52E−05 | −34.6302521 | Hypo-methylated | PCa\|NT DMRs |
| chr10 | 105376624 | 105376761 | 3.51E−05 | 25.56043956 | Hyper-methylated | PCa\|NT DMRs |
| chr10 | 105423558 | 105423915 | 2.06E−05 | −38.65544218 | Hypo-methylated | PCa\|NT DMRs |
| chr10 | 106081178 | 106097511 | 0.000775154 | 31.20734127 | Hyper-methylated | PCa\|NT DMRs |
| chr10 | 106174588 | 106195165 | 1.76E−06 | −35.39285714 | Hypo-methylated | PCa\|NT DMRs |
| chr10 | 110092420 | 110187102 | 0.00266883 | −29.59809524 | Hypo-methylated | PCa\|NT DMRs |
| chr10 | 111766841 | 111767609 | 1.29E−05 | 25.30672269 | Hyper-methylated | PCa\|NT DMRs |
| chr10 | 112193490 | 112193856 | 0.00641359 | −41.91666667 | Hypo-methylated | PCa\|NT DMRs |
| chr10 | 112838347 | 112847422 | 5.13E−14 | 28.0691358 | Hyper-methylated | PCa\|NT DMRs |
| chr10 | 114059288 | 114074175 | 0.00891501 | −20.15963719 | Hypo-methylated | PCa\|NT DMRs |
| chr10 | 114823362 | 114833756 | 0.004236974 | −25.82857143 | Hypo-methylated | PCa\|NT DMRs |
| chr10 | 115033264 | 115033784 | 0.001181579 | 39.10204082 | Hyper-methylated | PCa\|NT DMRs |
| chr10 | 115279121 | 115279297 | 0.001969641 | −34.27380952 | Hypo-methylated | PCa\|NT DMRs |
| chr10 | 115897350 | 115925464 | 0.005513661 | −23.8170068 | Hypo-methylated | PCa\|NT DMRs |
| chr10 | 115940219 | 115940529 | 0.000205174 | −54.41964286 | Hypo-methylated | PCa\|NT DMRs |
| chr10 | 116162884 | 116162984 | 0.001969641 | 47.14285714 | Hyper-methylated | PCa\|NT DMRs |
| chr10 | 116164640 | 116165087 | 0.003541715 | 20.3415873 | Hyper-methylated | PCa\|NT DMRs |
| chr10 | 116307367 | 116323944 | 1.26E−05 | 37.43968254 | Hyper-methylated | PCa\|NT DMRs |
| chr10 | 117591036 | 117997714 | 4.16E−23 | −26.63263985 | Hypo-methylated | PCa\|NT DMRs |
| chr10 | 118030475 | 118031355 | 5.93E−19 | 20.07562657 | Hyper-methylated | PCa\|NT DMRs |
| chr10 | 118345648 | 118364310 | 0.001969641 | −35.20408163 | Hypo-methylated | PCa\|NT DMRs |
| chr10 | 118429481 | 118429953 | 0.000251303 | 22.97732426 | Hyper-methylated | PCa\|NT DMRs |
| chr10 | 118464403 | 118464683 | 0.003400006 | −35.97959184 | Hypo-methylated | PCa\|NT DMRs |
| chr10 | 118891071 | 118896117 | 1.75E−31 | 24.41024259 | Hyper-methylated | PCa\|NT DMRs |
| chr10 | 118927246 | 118927464 | 1.29E−05 | 24.60022676 | Hyper-methylated | PCa\|NT DMRs |
| chr10 | 118928583 | 118928762 | 0.001969641 | 27.19047619 | Hyper-methylated | PCa\|NT DMRs |
| chr10 | 119204972 | 119208689 | 0.006096657 | −26.94444444 | Hypo-methylated | PCa\|NT DMRs |
| chr10 | 119291157 | 119292588 | 4.20E−07 | 28.05590062 | Hyper-methylated | PCa\|NT DMRs |
| chr10 | 119296516 | 119296695 | 6.90E−05 | 24.8968254 | Hyper-methylated | PCa\|NT DMRs |
| chr10 | 119296847 | 119296894 | 0.003272154 | 25.23214286 | Hyper-methylated | PCa\|NT DMRs |
| chr10 | 119304380 | 119310965 | 4.38E−32 | 26.43669951 | Hyper-methylated | PCa\|NT DMRs |
| chr10 | 119444256 | 119444642 | 0.000260936 | −20.97142857 | Hypo-methylated | PCa\|NT DMRs |
| chr10 | 119650134 | 119681020 | 0.00064571 | −28.05510204 | Hypo-methylated | PCa\|NT DMRs |
| chr10 | 120515032 | 120515437 | 0.005375157 | 30.2414966 | Hyper-methylated | PCa\|NT DMRs |
| chr10 | 121030606 | 121030663 | 0.008482405 | 35.16190476 | Hyper-methylated | PCa\|NT DMRs |
| chr10 | 121066077 | 121066241 | 0.005968871 | 51.57142857 | Hyper-methylated | PCa\|NT DMRs |
| chr10 | 121073487 | 121073656 | 0.001969641 | 48.5 | Hyper-methylated | PCa\|NT DMRs |
| chr10 | 121127442 | 121127615 | 0.000840726 | 33.69047619 | Hyper-methylated | PCa\|NT DMRs |
| chr10 | 121252647 | 121255786 | 0.000424658 | 28.60090703 | Hyper-methylated | PCa\|NT DMRs |
| chr10 | 121445679 | 121445881 | 0.000652677 | −35.41071429 | Hypo-methylated | PCa\|NT DMRs |
| chr10 | 122708684 | 122724071 | 7.99E−07 | 36.90026455 | Hyper-methylated | PCa\|NT DMRs |
| chr10 | 122739457 | 122759005 | 0.005646573 | 40.21428571 | Hyper-methylated | PCa\|NT DMRs |
| chr10 | 123229472 | 123250537 | 0.001254235 | −41.46483516 | Hypo-methylated | PCa\|NT DMRs |
| chr10 | 123333228 | 123333456 | 0.000118733 | −42.40899471 | Hypo-methylated | PCa\|NT DMRs |
| chr10 | 123733690 | 123733844 | 0.001969641 | −22.45 | Hypo-methylated | PCa\|NT DMRs |
| chr10 | 123778434 | 123781741 | 3.20E−05 | −42.08644689 | Hypo-methylated | PCa\|NT DMRs |
| chr10 | 123873842 | 123874281 | 0.005968871 | −35.12698413 | Hypo-methylated | PCa\|NT DMRs |
| chr10 | 124058195 | 124059587 | 0.005513661 | −37.90612245 | Hypo-methylated | PCa\|NT DMRs |
| chr10 | 124233801 | 124234900 | 0.006589535 | 28.50714286 | Hyper-methylated | PCa\|NT DMRs |
| chr10 | 124279446 | 124279587 | 2.58E−05 | −32.86095238 | Hypo-methylated | PCa\|NT DMRs |
| chr10 | 124487395 | 124557584 | 7.75E−10 | −25.18452381 | Hypo-methylated | PCa\|NT DMRs |
| chr10 | 125220997 | 125228559 | 0.00017871 | −28.61 | Hypo-methylated | PCa\|NT DMRs |
| chr10 | 125234404 | 125239922 | 0.002437742 | −26.95185185 | Hypo-methylated | PCa\|NT DMRs |
| chr10 | 125310903 | 125411063 | 1.17E−18 | −31.61390977 | Hypo-methylated | PCa\|NT DMRs |
| chr10 | 125426266 | 125426294 | 0.001115302 | 23.23214286 | Hyper-methylated | PCa\|NT DMRs |
| chr10 | 125428601 | 125650354 | 8.22E−24 | −33.66460317 | Hypo-methylated | PCa\|NT DMRs |
| chr10 | 125673698 | 125727334 | 2.61E−07 | −21.44091711 | Hypo-methylated | PCa\|NT DMRs |
| chr10 | 125803689 | 125815013 | 0.000874558 | −27.55974026 | Hypo-methylated | PCa\|NT DMRs |
| chr10 | 125830141 | 125847725 | 0.001563412 | −28.8802005 | Hypo-methylated | PCa\|NT DMRs |
| chr10 | 125851162 | 125851825 | 4.51E−18 | 27.63044496 | Hyper-methylated | PCa\|NT DMRs |
| chr10 | 125922278 | 125933051 | 8.50E−10 | −20.69256678 | Hypo-methylated | PCa\|NT DMRs |
| chr10 | 126077501 | 126077549 | 0.000481535 | 27.92063492 | Hyper-methylated | PCa\|NT DMRs |
| chr10 | 126106146 | 126106294 | 0.00064571 | −35.84693878 | Hypo-methylated | PCa\|NT DMRs |
| chr10 | 126222956 | 126224655 | 0.001181579 | 39.86938776 | Hyper-methylated | PCa\|NT DMRs |
| chr10 | 126254410 | 126254770 | 0.00641359 | −32.50793651 | Hypo-methylated | PCa\|NT DMRs |
| chr10 | 126300445 | 126303557 | 0.005295761 | 34.53030303 | Hyper-methylated | PCa\|NT DMRs |
| chr10 | 126308381 | 126308821 | 0.005438982 | 24.36428571 | Hyper-methylated | PCa\|NT DMRs |
| chr10 | 126363463 | 126366197 | 0.003921091 | 23.62770563 | Hyper-methylated | PCa\|NT DMRs |
| chr10 | 126678095 | 126683437 | 0.005968871 | −36.06296296 | Hypo-methylated | PCa\|NT DMRs |
| chr10 | 126686860 | 126690570 | 2.24E−06 | −25.75517241 | Hypo-methylated | PCa\|NT DMRs |
| chr10 | 126812260 | 126823549 | 0.000751352 | −35.7070028 | Hypo-methylated | PCa\|NT DMRs |
| chr10 | 126850914 | 126851130 | 8.96E−05 | 35.29761905 | Hyper-methylated | PCa\|NT DMRs |
| chr10 | 127050542 | 127059592 | 0.005601663 | 21.05569358 | Hyper-methylated | PCa\|NT DMRs |
| chr10 | 127462344 | 127462851 | 1.69E−13 | 23.84531746 | Hyper-methylated | PCa\|NT DMRs |
| chr10 | 127914914 | 127938198 | 1.03E−05 | −30.49722222 | Hypo-methylated | PCa\|NT DMRs |
| chr10 | 128077288 | 128077792 | 5.62E−06 | 33.77931034 | Hyper-methylated | PCa\|NT DMRs |
| chr10 | 129733149 | 129733592 | 0.001139334 | 25.3281746 | Hyper-methylated | PCa\|NT DMRs |
| chr10 | 129770270 | 129790276 | 0.001116719 | 21.9562212 | Hyper-methylated | PCa\|NT DMRs |
| chr10 | 129858596 | 129864571 | 0.002233556 | 23.72704082 | Hyper-methylated | PCa\|NT DMRs |
| chr10 | 130020722 | 130020924 | 0.00064571 | −41.18367347 | Hypo-methylated | PCa\|NT DMRs |
| chr10 | 130064035 | 130064194 | 0.00179283 | 20.86147186 | Hyper-methylated | PCa\|NT DMRs |
| chr10 | 130084659 | 130085481 | 3.29E−07 | 20.1031746 | Hyper-methylated | PCa\|NT DMRs |
| chr10 | 130278887 | 130280004 | 2.49E−06 | −33.42162698 | Hypo-methylated | PCa\|NT DMRs |
| chr10 | 130482299 | 130489627 | 0.002437742 | −21.74232804 | Hypo-methylated | PCa\|NT DMRs |
| chr10 | 130594618 | 130600266 | 0.005968871 | −38.6047619 | Hypo-methylated | PCa\|NT DMRs |
| chr10 | 130832358 | 130832419 | 5.84E−05 | 32.72619048 | Hyper-methylated | PCa\|NT DMRs |
| chr10 | 131193314 | 131194474 | 0.000200126 | −24.43772894 | Hypo-methylated | PCa\|NT DMRs |
| chr10 | 131506200 | 131506279 | 0.005968871 | −58.62857143 | Hypo-methylated | PCa\|NT DMRs |
| chr10 | 131755588 | 131758298 | 1.32E−38 | 22.04497354 | Hyper-methylated | PCa\|NT DMRs |
| chr10 | 131826134 | 131826673 | 0.003448944 | −38.38367347 | Hypo-methylated | PCa\|NT DMRs |
| chr10 | 131861385 | 131861677 | 0.000188936 | −25.50595238 | Hypo-methylated | PCa\|NT DMRs |
| chr10 | 132019960 | 132020489 | 0.000118733 | −44.65873016 | Hypo-methylated | PCa\|NT DMRs |
| chr10 | 132119797 | 132128822 | 0.001083007 | −20.78031746 | Hypo-methylated | PCa\|NT DMRs |
| chr10 | 132154720 | 132162308 | 0.000815653 | −26.23730159 | Hypo-methylated | PCa\|NT DMRs |
| chr10 | 132177762 | 132308583 | 8.20E−23 | −24.79236948 | Hypo-methylated | PCa\|NT DMRs |
| chr10 | 132329756 | 132365705 | 6.88E−13 | −23.28377425 | Hypo-methylated | PCa\|NT DMRs |
| chr10 | 132448255 | 132452879 | 1.67E−05 | −34.06547619 | Hypo-methylated | PCa\|NT DMRs |
| chr10 | 132464783 | 132488867 | 0.002433478 | −30.73809524 | Hypo-methylated | PCa\|NT DMRs |
| chr10 | 132539506 | 132560543 | 3.12E−08 | −28.05044092 | Hypo-methylated | PCa\|NT DMRs |
| chr10 | 132818102 | 132847207 | 7.98E−14 | −28.92063492 | Hypo-methylated | PCa\|NT DMRs |
| chr10 | 132883449 | 132883666 | 0.00064571 | 30.20068027 | Hyper-methylated | PCa\|NT DMRs |
| chr10 | 133058588 | 133070062 | 3.81E−05 | −22.45378151 | Hypo-methylated | PCa\|NT DMRs |
| chr10 | 133357723 | 133454857 | 1.86E−26 | −29.24356553 | Hypo-methylated | PCa\|NT DMRs |
| chr10 | 133476648 | 133521931 | 3.23E−15 | −27.00648926 | Hypo-methylated | PCa\|NT DMRs |
| chr10 | 133619180 | 133635888 | 0.003578734 | −23.26834734 | Hypo-methylated | PCa\|NT DMRs |
| chr10 | 133638698 | 133737519 | 9.08E−22 | −24.98830409 | Hypo-methylated | PCa\|NT DMRs |
| chr10 | 133837293 | 133837348 | 0.000377317 | −30.51785714 | Hypo-methylated | PCa\|NT DMRs |
| chr10 | 133861842 | 133861990 | 0.001969641 | −48.38095238 | Hypo-methylated | PCa\|NT DMRs |
| chr10 | 133890732 | 133892633 | 8.30E−06 | 24.84970238 | Hyper-methylated | PCa\|NT DMRs |
| chr10 | 133946539 | 133946882 | 0.000810017 | −22.04389234 | Hypo-methylated | PCa\|NT DMRs |
| chr10 | 134001067 | 134002033 | 1.51E−09 | 24.50027473 | Hyper-methylated | PCa\|NT DMRs |
| chr10 | 134063331 | 134063676 | 0.000572385 | 22.51666667 | Hyper-methylated | PCa\|NT DMRs |
| chr10 | 134079332 | 134079479 | 0.00064571 | −53.9122449 | Hypo-methylated | PCa\|NT DMRs |
| chr10 | 134107089 | 134107362 | 0.001083007 | −45.15952381 | Hypo-methylated | PCa\|NT DMRs |
| chr10 | 134220460 | 134226000 | 0.000128082 | −28.495671 | Hypo-methylated | PCa\|NT DMRs |
| chr10 | 134256135 | 134262212 | 1.99E−05 | 26.84824798 | Hyper-methylated | PCa\|NT DMRs |
| chr10 | 134310933 | 134311115 | 0.00064571 | 38.84013605 | Hyper-methylated | PCa\|NT DMRs |
| chr10 | 134331048 | 134331115 | 0.005968871 | 20.71428571 | Hyper-methylated | PCa\|NT DMRs |
| chr10 | 134331816 | 134332730 | 0.001175696 | −21.54089636 | Hypo-methylated | PCa\|NT DMRs |
| chr10 | 134392611 | 134392984 | 2.75E−05 | 25.68941799 | Hyper-methylated | PCa\|NT DMRs |
| chr10 | 134499085 | 134499409 | 1.42E−05 | −54.55952381 | Hypo-methylated | PCa\|NT DMRs |
| chr10 | 134577650 | 134578521 | 0.000572385 | −35.34880952 | Hypo-methylated | PCa\|NT DMRs |
| chr10 | 134772885 | 134774108 | 3.98E−06 | −30.56340852 | Hypo-methylated | PCa\|NT DMRs |
| chr10 | 135071741 | 135074493 | 0.000163436 | 29.90833333 | Hyper-methylated | PCa\|NT DMRs |
| chr10 | 135087840 | 135090821 | 3.60E−19 | 31.87207792 | Hyper-methylated | PCa\|NT DMRs |
| chr10 | 135148215 | 135148379 | 0.00569382 | 25.25142857 | Hyper-methylated | PCa\|NT DMRs |
| chr10 | 135170769 | 135171524 | 9.80E−21 | 21.35485714 | Hyper-methylated | PCa\|NT DMRs |
| chr10 | 135173233 | 135174232 | 3.44E−06 | −50.9360119 | Hypo-methylated | PCa\|NT DMRs |
| chr10 | 135177818 | 135186510 | 2.49E−06 | −27.24642857 | Hypo-methylated | PCa\|NT DMRs |
| chr10 | 135193212 | 135193966 | 2.31E−05 | −21.26380952 | Hypo-methylated | PCa\|NT DMRs |
| chr10 | 135202490 | 135205539 | 0.000205174 | 33.59642857 | Hyper-methylated | PCa\|NT DMRs |
| chr10 | 135278717 | 135279026 | 5.45E−08 | 20.90742297 | Hyper-methylated | PCa\|NT DMRs |
| chr11 | 189759 | 197036 | 1.04E−05 | −26.84285714 | Hypo-methylated | PCa\|NT DMRs |
| chr11 | 296220 | 300915 | 2.83E−07 | −21.22723475 | Hypo-methylated | PCa\|NT DMRs |
| chr11 | 326026 | 326953 | 0.001076343 | 32.65367965 | Hyper-methylated | PCa\|NT DMRs |
| chr11 | 355557 | 355907 | 0.001129594 | 21.96938776 | Hyper-methylated | PCa\|NT DMRs |
| chr11 | 381303 | 382928 | 2.50E−05 | 32.16513057 | Hyper-methylated | PCa\|NT DMRs |
| chr11 | 391004 | 391323 | 0.007679542 | −22.96938776 | Hypo-methylated | PCa\|NT DMRs |
| chr11 | 409751 | 410268 | 2.03E−06 | −41.08 | Hypo-methylated | PCa\|NT DMRs |
| chr11 | 415120 | 416269 | 5.87E−08 | 47.19690476 | Hyper-methylated | PCa\|NT DMRs |
| chr11 | 428780 | 430335 | 0.000284668 | −20.79029304 | Hypo-methylated | PCa\|NT DMRs |
| chr11 | 438565 | 438623 | 0.003400006 | −27.17006803 | Hypo-methylated | PCa\|NT DMRs |
| chr11 | 457304 | 457482 | 0.00064571 | 46.06122449 | Hyper-methylated | PCa\|NT DMRs |
| chr11 | 468308 | 471390 | 2.42E−05 | −27.13250518 | Hypo-methylated | PCa\|NT DMRs |
| chr11 | 518565 | 518649 | 5.94E−06 | −39.21038961 | Hypo-methylated | PCa\|NT DMRs |
| chr11 | 569079 | 569585 | 0.000190658 | 28.39350649 | Hyper-methylated | PCa\|NT DMRs |
| chr11 | 611583 | 613053 | 4.56E−05 | −22.07142857 | Hypo-methylated | PCa\|NT DMRs |
| chr11 | 613978 | 615335 | 2.66E−11 | −42.66214896 | Hypo-methylated | PCa\|NT DMRs |
| chr11 | 637015 | 637876 | 4.60E−13 | 20.09545788 | Hyper-methylated | PCa\|NT DMRs |
| chr11 | 696745 | 706044 | 5.21E−05 | −27.78036929 | Hypo-methylated | PCa\|NT DMRs |
| chr11 | 772670 | 773252 | 0.003583021 | −23.78888889 | Hypo-methylated | PCa\|NT DMRs |
| chr11 | 782976 | 783617 | 0.000146826 | −34.75214286 | Hypo-methylated | PCa\|NT DMRs |
| chr11 | 787739 | 788443 | 0.001562883 | 22.08979592 | Hyper-methylated | PCa\|NT DMRs |
| chr11 | 794755 | 795583 | 6.43E−05 | −21.10294785 | Hypo-methylated | PCa\|NT DMRs |
| chr11 | 823706 | 826239 | 1.13E−15 | −49.31398176 | Hypo-methylated | PCa\|NT DMRs |
| chr11 | 840291 | 841636 | 0.001015461 | −22.35714286 | Hypo-methylated | PCa\|NT DMRs |
| chr11 | 850196 | 851049 | 3.78E−06 | −30.76210317 | Hypo-methylated | PCa\|NT DMRs |
| chr11 | 858671 | 862859 | 7.72E−13 | −28.12434457 | Hypo-methylated | PCa\|NT DMRs |
| chr11 | 925384 | 925430 | 0.001969641 | 33.80952381 | Hyper-methylated | PCa\|NT DMRs |
| chr11 | 968922 | 971653 | 0.003694294 | 28.91575092 | Hyper-methylated | PCa\|NT DMRs |
| chr11 | 1029129 | 1030777 | 5.90E−07 | −23.60426587 | Hypo-methylated | PCa\|NT DMRs |
| chr11 | 1041355 | 1042259 | 0.002803541 | −30.13730159 | Hypo-methylated | PCa\|NT DMRs |
| chr11 | 1043886 | 1043952 | 6.42E−05 | −45.79365079 | Hypo-methylated | PCa\|NT DMRs |
| chr11 | 1052880 | 1055266 | 3.70E−06 | −36.09137709 | Hypo-methylated | PCa\|NT DMRs |
| chr11 | 1079567 | 1079809 | 0.000354065 | −33.6968254 | Hypo-methylated | PCa\|NT DMRs |
| chr11 | 1081684 | 1081848 | 5.52E−05 | −36.48051948 | Hypo-methylated | PCa\|NT DMRs |
| chr11 | 1084299 | 1084406 | 0.000481535 | −22.27777778 | Hypo-methylated | PCa\|NT DMRs |
| chr11 | 1145197 | 1146854 | 3.86E−10 | 41.59300595 | Hyper-methylated | PCa\|NT DMRs |
| chr11 | 1216195 | 1216973 | 0.000344011 | −26.61176471 | Hypo-methylated | PCa\|NT DMRs |
| chr11 | 1217280 | 1219755 | 7.06E−12 | −22.22213868 | Hypo-methylated | PCa\|NT DMRs |
| chr11 | 1241256 | 1241568 | 0.001800067 | −21.88958333 | Hypo-methylated | PCa\|NT DMRs |
| chr11 | 1243817 | 1245364 | 3.77E−05 | −27.95142857 | Hypo-methylated | PCa\|NT DMRs |
| chr11 | 1260215 | 1260592 | 0.000392715 | −33.91714286 | Hypo-methylated | PCa\|NT DMRs |
| chr11 | 1270339 | 1271077 | 0.000205174 | −30.27380952 | Hypo-methylated | PCa\|NT DMRs |
| chr11 | 1331419 | 1331636 | 0.000251944 | −20.1443609 | Hypo-methylated | PCa\|NT DMRs |
| chr11 | 1332016 | 1332394 | 0.000858283 | 52.56493506 | Hyper-methylated | PCa\|NT DMRs |
| chr11 | 1358178 | 1358819 | 1.16E−22 | 23.79791332 | Hyper-methylated | PCa\|NT DMRs |
| chr11 | 1361518 | 1365001 | 0.003314163 | −30.60095238 | Hypo-methylated | PCa\|NT DMRs |
| chr11 | 1375302 | 1375865 | 2.37E−05 | −24.1047619 | Hypo-methylated | PCa\|NT DMRs |
| chr11 | 1409935 | 1410134 | 0.000974795 | 32.70595238 | Hyper-methylated | PCa\|NT DMRs |
| chr11 | 1412450 | 1412572 | 0.000377317 | 20.80952381 | Hyper-methylated | PCa\|NT DMRs |
| chr11 | 1427510 | 1437123 | 0.000157373 | −24.1021164 | Hypo-methylated | PCa\|NT DMRs |
| chr11 | 1455363 | 1455574 | 0.000632596 | −43.42857143 | Hypo-methylated | PCa\|NT DMRs |
| chr11 | 1470285 | 1470367 | 0.000172237 | 41.48571429 | Hyper-methylated | PCa\|NT DMRs |
| chr11 | 1507159 | 1508462 | 0.000164894 | −22.0543514 | Hypo-methylated | PCa\|NT DMRs |
| chr11 | 1524032 | 1524769 | 0.006407159 | −28.95670996 | Hypo-methylated | PCa\|NT DMRs |
| chr11 | 1536165 | 1537082 | 0.005955401 | −24.87359307 | Hypo-methylated | PCa\|NT DMRs |
| chr11 | 1568900 | 1569417 | 5.19E−07 | 26.61527778 | Hyper-methylated | PCa\|NT DMRs |
| chr11 | 1577228 | 1578737 | 4.17E−16 | 38.94197802 | Hyper-methylated | PCa\|NT DMRs |
| chr11 | 1591405 | 1592675 | 0.007403789 | 20.15892857 | Hyper-methylated | PCa\|NT DMRs |
| chr11 | 1629694 | 1644607 | 0.000392715 | −23.14714286 | Hypo-methylated | PCa\|NT DMRs |
| chr11 | 1662582 | 1664538 | 3.36E−06 | −28.01014493 | Hypo-methylated | PCa\|NT DMRs |
| chr11 | 1665947 | 1666155 | 5.94E−06 | −31.73593074 | Hypo-methylated | PCa\|NT DMRs |
| chr11 | 1687822 | 1705621 | 1.57E−07 | −21.01428571 | Hypo-methylated | PCa\|NT DMRs |
| chr11 | 1749330 | 1766467 | 1.26E−05 | −28.90062657 | Hypo-methylated | PCa\|NT DMRs |
| chr11 | 1767793 | 1768079 | 0.000169316 | 21.58730159 | Hyper-methylated | PCa\|NT DMRs |
| chr11 | 1772276 | 1776901 | 6.85E−07 | 20.82103767 | Hyper-methylated | PCa\|NT DMRs |
| chr11 | 1778046 | 1782746 | 1.49E−11 | 22.96197279 | Hyper-methylated | PCa\|NT DMRs |
| chr11 | 1802030 | 1802436 | 0.00064571 | 36.64285714 | Hyper-methylated | PCa\|NT DMRs |
| chr11 | 1846375 | 1846611 | 0.003448944 | 33.84798535 | Hyper-methylated | PCa\|NT DMRs |
| chr11 | 1879145 | 1879521 | 0.000205174 | −27.19642857 | Hypo-methylated | PCa\|NT DMRs |
| chr11 | 1884626 | 1886444 | 3.46E−05 | −33.72914286 | Hypo-methylated | PCa\|NT DMRs |
| chr11 | 1909030 | 1918969 | 5.47E−13 | 34.57640693 | Hyper-methylated | PCa\|NT DMRs |
| chr11 | 1987847 | 1990174 | 7.32E−07 | −20.57021792 | Hypo-methylated | PCa\|NT DMRs |
| chr11 | 1991933 | 1992320 | 0.00013778 | −20.83333333 | Hypo-methylated | PCa\|NT DMRs |
| chr11 | 1994362 | 1995123 | 3.02E−06 | −24.99821429 | Hypo-methylated | PCa\|NT DMRs |
| chr11 | 2060636 | 2092371 | 0.00068166 | −20.92916667 | Hypo-methylated | PCa\|NT DMRs |
| chr11 | 2118862 | 2119159 | 0.001310211 | −26.58571429 | Hypo-methylated | PCa\|NT DMRs |
| chr11 | 2143574 | 2144335 | 3.77E−05 | −29.34223602 | Hypo-methylated | PCa\|NT DMRs |
| chr11 | 2214934 | 2215334 | 0.000176268 | −20.38235294 | Hypo-methylated | PCa\|NT DMRs |
| chr11 | 2223474 | 2223662 | 0.007959104 | −35.00544218 | Hypo-methylated | PCa\|NT DMRs |
| chr11 | 2278449 | 2278938 | 4.89E−08 | −49.65909091 | Hypo-methylated | PCa\|NT DMRs |
| chr11 | 2310683 | 2320775 | 0.007526322 | −23.01071429 | Hypo-methylated | PCa\|NT DMRs |
| chr11 | 2321699 | 2324965 | 0.002211645 | 33.88278388 | Hyper-methylated | PCa\|NT DMRs |
| chr11 | 2329041 | 2329457 | 0.005968871 | −27.06190476 | Hypo-methylated | PCa\|NT DMRs |
| chr11 | 2397303 | 2397774 | 6.69E−05 | −48.85238095 | Hypo-methylated | PCa\|NT DMRs |
| chr11 | 2425925 | 2428586 | 6.80E−09 | −34.86359447 | Hypo-methylated | PCa\|NT DMRs |
| chr11 | 2443237 | 2448964 | 4.16E−09 | −20.57718254 | Hypo-methylated | PCa\|NT DMRs |
| chr11 | 2487303 | 2488886 | 0.000715461 | 31.28571429 | Hyper-methylated | PCa\|NT DMRs |
| chr11 | 2554914 | 2555248 | 2.86E−05 | 49.56043956 | Hyper-methylated | PCa\|NT DMRs |
| chr11 | 2769117 | 2769207 | 0.005118278 | 22.6984127 | Hyper-methylated | PCa\|NT DMRs |
| chr11 | 2795863 | 2799884 | 0.000278843 | 22.31587302 | Hyper-methylated | PCa\|NT DMRs |
| chr11 | 2801145 | 2810152 | 4.05E−07 | 40.71005291 | Hyper-methylated | PCa\|NT DMRs |
| chr11 | 2847158 | 2849208 | 1.16E−05 | 39.02689076 | Hyper-methylated | PCa\|NT DMRs |
| chr11 | 2859574 | 2859934 | 0.001699622 | 22.03736264 | Hyper-methylated | PCa\|NT DMRs |
| chr11 | 2860969 | 2862483 | 0.001850915 | −37.5952381 | Hypo-methylated | PCa\|NT DMRs |
| chr11 | 2870712 | 2871458 | 0.001452156 | −26.44867725 | Hypo-methylated | PCa\|NT DMRs |
| chr11 | 2905051 | 2905522 | 7.59E−10 | 23.59047619 | Hyper-methylated | PCa\|NT DMRs |
| chr11 | 2911274 | 2912409 | 5.46E−05 | −38.56190476 | Hypo-methylated | PCa\|NT DMRs |
| chr11 | 2952174 | 2952440 | 0.004886033 | −34.68681319 | Hypo-methylated | PCa\|NT DMRs |
| chr11 | 3104576 | 3104685 | 0.000354065 | 34.74973545 | Hyper-methylated | PCa\|NT DMRs |
| chr11 | 3236759 | 3237763 | 0.000354065 | −36.81904762 | Hypo-methylated | PCa\|NT DMRs |
| chr11 | 3241803 | 3243314 | 2.01E−05 | −26.47904762 | Hypo-methylated | PCa\|NT DMRs |
| chr11 | 3244384 | 3244487 | 0.004037929 | 28.75986395 | Hyper-methylated | PCa\|NT DMRs |
| chr11 | 3245602 | 3246417 | 0.000122432 | −20.97402597 | Hypo-methylated | PCa\|NT DMRs |
| chr11 | 3497927 | 3498254 | 1.97E−05 | −50.68095238 | Hypo-methylated | PCa\|NT DMRs |
| chr11 | 4233365 | 4233428 | 6.42E−05 | 32.06402116 | Hyper-methylated | PCa\|NT DMRs |
| chr11 | 6340446 | 6341909 | 9.20E−08 | 30.52438095 | Hyper-methylated | PCa\|NT DMRs |
| chr11 | 6718497 | 6938263 | 8.59E−05 | −24.72494331 | Hypo-methylated | PCa\|NT DMRs |
| chr11 | 7331763 | 7404387 | 0.000215808 | −25.83809524 | Hypo-methylated | PCa\|NT DMRs |
| chr11 | 8378642 | 8378865 | 5.94E−06 | −26.15844156 | Hypo-methylated | PCa\|NT DMRs |
| chr11 | 8737118 | 8737725 | 0.000279449 | 36.84835165 | Hyper-methylated | PCa\|NT DMRs |
| chr11 | 8743062 | 8743397 | 0.005968871 | −47.28571429 | Hypo-methylated | PCa\|NT DMRs |
| chr11 | 9112531 | 9115613 | 3.14E−16 | 23.1922619 | Hyper-methylated | PCa\|NT DMRs |
| chr11 | 9352573 | 9352746 | 0.005968871 | −21.94285714 | Hypo-methylated | PCa\|NT DMRs |
| chr11 | 9761857 | 9762057 | 0.005968871 | −48.02380952 | Hypo-methylated | PCa\|NT DMRs |
| chr11 | 9780693 | 9780891 | 0.001756926 | 27.67532468 | Hyper-methylated | PCa\|NT DMRs |
| chr11 | 10373208 | 10374117 | 0.000513415 | −30.88265306 | Hypo-methylated | PCa\|NT DMRs |
| chr11 | 10477821 | 10481018 | 0.002822916 | 29.07738095 | Hyper-methylated | PCa\|NT DMRs |
| chr11 | 10696385 | 10696570 | 0.001969641 | 32.70408163 | Hyper-methylated | PCa\|NT DMRs |
| chr11 | 11577882 | 11577963 | 0.00064571 | 20.2244898 | Hyper-methylated | PCa\|NT DMRs |
| chr11 | 11602473 | 11602883 | 0.00064571 | −55.06530612 | Hypo-methylated | PCa\|NT DMRs |
| chr11 | 12133006 | 12147266 | 0.000457395 | −27.72710623 | Hypo-methylated | PCa\|NT DMRs |
| chr11 | 12246231 | 12246580 | 0.002097193 | 47.28571429 | Hyper-methylated | PCa\|NT DMRs |
| chr11 | 13259400 | 13260165 | 0.001241245 | −21.4984127 | Hypo-methylated | PCa\|NT DMRs |
| chr11 | 14014146 | 14099328 | 0.004960253 | −24.18190476 | Hypo-methylated | PCa\|NT DMRs |
| chr11 | 14444083 | 14445441 | 0.005968871 | 42.85714286 | Hyper-methylated | PCa\|NT DMRs |
| chr11 | 15672498 | 15672803 | 2.86E−05 | 39.82234432 | Hyper-methylated | PCa\|NT DMRs |
| chr11 | 16812592 | 16812647 | 0.00023371 | 27.34761905 | Hyper-methylated | PCa\|NT DMRs |
| chr11 | 17034484 | 17034890 | 0.001795658 | −20.67965368 | Hypo-methylated | PCa\|NT DMRs |
| chr11 | 17497511 | 17497842 | 6.02E−06 | 27.45804989 | Hyper-methylated | PCa\|NT DMRs |
| chr11 | 17610719 | 17613146 | 2.86E−07 | −24.91836735 | Hypo-methylated | PCa\|NT DMRs |
| chr11 | 18067693 | 18067898 | 1.66E−10 | 20.13095238 | Hyper-methylated | PCa\|NT DMRs |
| chr11 | 18415095 | 18415164 | 0.005968871 | 45.14285714 | Hyper-methylated | PCa\|NT DMRs |
| chr11 | 18660236 | 18690509 | 0.002463549 | −20.83201058 | Hypo-methylated | PCa\|NT DMRs |
| chr11 | 18814124 | 18814307 | 0.000377317 | 31.57738095 | Hyper-methylated | PCa\|NT DMRs |
| chr11 | 19331335 | 19366473 | 0.004553481 | −32.04497354 | Hypo-methylated | PCa\|NT DMRs |
| chr11 | 19799412 | 19799663 | 0.001042833 | 22.07380952 | Hyper-methylated | PCa\|NT DMRs |
| chr11 | 20234715 | 20339377 | 8.58E−07 | −34.75694444 | Hypo-methylated | PCa\|NT DMRs |
| chr11 | 21920677 | 22163687 | 3.43E−06 | −28.15416667 | Hypo-methylated | PCa\|NT DMRs |
| chr11 | 22473811 | 22623620 | 0.001241245 | −34.38498168 | Hypo-methylated | PCa\|NT DMRs |
| chr11 | 29105863 | 29849310 | 3.26E−12 | −30.26559524 | Hypo-methylated | PCa\|NT DMRs |
| chr11 | 31915052 | 32005742 | 0.00046742 | −28.36147186 | Hypo-methylated | PCa\|NT DMRs |
| chr11 | 32022513 | 32052791 | 0.005968871 | −39.17047619 | Hypo-methylated | PCa\|NT DMRs |
| chr11 | 32915068 | 32915702 | 2.15E−12 | 23.46084656 | Hyper-methylated | PCa\|NT DMRs |
| chr11 | 33471349 | 33526406 | 0.004553481 | −23.48730159 | Hypo-methylated | PCa\|NT DMRs |
| chr11 | 33677736 | 33718878 | 1.04E−05 | −21.64536341 | Hypo-methylated | PCa\|NT DMRs |
| chr11 | 33720593 | 33722029 | 7.03E−07 | 33.94659864 | Hyper-methylated | PCa\|NT DMRs |
| chr11 | 33840720 | 33843290 | 0.000974795 | −23.17857143 | Hypo-methylated | PCa\|NT DMRs |
| chr11 | 33850491 | 33850706 | 0.002467228 | 20.79285714 | Hyper-methylated | PCa\|NT DMRs |
| chr11 | 33890313 | 33890427 | 0.000377317 | 26.80357143 | Hyper-methylated | PCa\|NT DMRs |
| chr11 | 34176919 | 34177143 | 0.000477661 | −42.79365079 | Hypo-methylated | PCa\|NT DMRs |
| chr11 | 34190768 | 34191182 | 0.007338757 | −54.99047619 | Hypo-methylated | PCa\|NT DMRs |
| chr11 | 35327601 | 35327749 | 0.001907648 | 23.26190476 | Hyper-methylated | PCa\|NT DMRs |
| chr11 | 35441332 | 35442136 | 4.32E−16 | 36.50096852 | Hyper-methylated | PCa\|NT DMRs |
| chr11 | 36145373 | 36145511 | 0.008482405 | −62.08571429 | Hypo-methylated | PCa\|NT DMRs |
| chr11 | 36397568 | 36397612 | 0.005968871 | 44.03333333 | Hyper-methylated | PCa\|NT DMRs |
| chr11 | 38300432 | 38470399 | 1.42E−09 | −21.30297619 | Hypo-methylated | PCa\|NT DMRs |
| chr11 | 38609186 | 38656021 | 0.001111028 | −34.76984127 | Hypo-methylated | PCa\|NT DMRs |
| chr11 | 39471249 | 40201149 | 1.34E−24 | −30.77926587 | Hypo-methylated | PCa\|NT DMRs |
| chr11 | 43602594 | 43604909 | 3.38E−14 | 20.64880952 | Hyper-methylated | PCa\|NT DMRs |
| chr11 | 44541795 | 44542054 | 3.11E−06 | 20.23714286 | Hyper-methylated | PCa\|NT DMRs |
| chr11 | 44814303 | 44833307 | 0.008234951 | −23.04761905 | Hypo-methylated | PCa\|NT DMRs |
| chr11 | 44840127 | 44865219 | 1.16E−06 | −26.09077381 | Hypo-methylated | PCa\|NT DMRs |
| chr11 | 44948290 | 44951493 | 0.000715461 | −23.2202381 | Hypo-methylated | PCa\|NT DMRs |
| chr11 | 44960769 | 44961736 | 5.50E−07 | 26.83503401 | Hyper-methylated | PCa\|NT DMRs |
| chr11 | 45101759 | 45102447 | 0.001969641 | 48.71428571 | Hyper-methylated | PCa\|NT DMRs |
| chr11 | 45182876 | 45184743 | 0.00601179 | −24.23904762 | Hypo-methylated | PCa\|NT DMRs |
| chr11 | 45446393 | 45467192 | 0.00064571 | −34.62857143 | Hypo-methylated | PCa\|NT DMRs |
| chr11 | 45685821 | 45685918 | 0.005968871 | 41.71238095 | Hyper-methylated | PCa\|NT DMRs |
| chr11 | 45907522 | 45908075 | 0.002879175 | 21.08739496 | Hyper-methylated | PCa\|NT DMRs |
| chr11 | 45943130 | 45943839 | 3.45E−14 | −34.64606227 | Hypo-methylated | PCa\|NT DMRs |
| chr11 | 46366989 | 46367974 | 0.005968871 | 60.26190476 | Hyper-methylated | PCa\|NT DMRs |
| chr11 | 46372695 | 46384984 | 0.00021751 | 23.60044297 | Hyper-methylated | PCa\|NT DMRs |
| chr11 | 46564148 | 46564344 | 0.007560755 | −22.46011905 | Hypo-methylated | PCa\|NT DMRs |
| chr11 | 47206523 | 47207567 | 4.03E−05 | −23.77960928 | Hypo-methylated | PCa\|NT DMRs |
| chr11 | 47208766 | 47214114 | 0.000196134 | 31.24952381 | Hyper-methylated | PCa\|NT DMRs |
| chr11 | 47214207 | 47215955 | 0.003892841 | −30.33862434 | Hypo-methylated | PCa\|NT DMRs |
| chr11 | 47279157 | 47289256 | 0.000185736 | 25.37637363 | Hyper-methylated | PCa\|NT DMRs |
| chr11 | 47376715 | 47380422 | 9.33E−05 | −45.89047619 | Hypo-methylated | PCa\|NT DMRs |
| chr11 | 47396170 | 47417596 | 1.56E−22 | 42.33257576 | Hyper-methylated | PCa\|NT DMRs |
| chr11 | 47609825 | 47609937 | 0.005968871 | 49.96571429 | Hyper-methylated | PCa\|NT DMRs |
| chr11 | 48012330 | 48012379 | 0.008482405 | −27 | Hypo-methylated | PCa\|NT DMRs |
| chr11 | 48023591 | 48057012 | 0.002885118 | 25.25380952 | Hyper-methylated | PCa\|NT DMRs |
| chr11 | 48101848 | 48103102 | 8.94E−05 | 22.56255411 | Hyper-methylated | PCa\|NT DMRs |
| chr11 | 49031974 | 49069341 | 0.005968871 | −24.81428571 | Hypo-methylated | PCa\|NT DMRs |
| chr11 | 49455099 | 49455940 | 0.001115302 | −31.80119048 | Hypo-methylated | PCa\|NT DMRs |
| chr11 | 49912911 | 49913484 | 0.000847246 | −30.07582418 | Hypo-methylated | PCa\|NT DMRs |
| chr11 | 49915274 | 50180943 | 3.56E−12 | −31.34031746 | Hypo-methylated | PCa\|NT DMRs |
| chr11 | 50221563 | 50257289 | 1.08E−14 | −28.44088889 | Hypo-methylated | PCa\|NT DMRs |
| chr11 | 56557728 | 56800416 | 2.33E−12 | −32.24821429 | Hypo-methylated | PCa\|NT DMRs |
| chr11 | 57147640 | 57182673 | 1.50E−05 | −23.68035714 | Hypo-methylated | PCa\|NT DMRs |
| chr11 | 57225287 | 57225426 | 0.001969641 | 34.08843537 | Hyper-methylated | PCa\|NT DMRs |
| chr11 | 57235346 | 57235492 | 6.94E−06 | −59.11428571 | Hypo-methylated | PCa\|NT DMRs |
| chr11 | 57478704 | 57478950 | 0.00179283 | −28.93452381 | Hypo-methylated | PCa\|NT DMRs |
| chr11 | 58376323 | 58377458 | 0.00329109 | 37.36363636 | Hyper-methylated | PCa\|NT DMRs |
| chr11 | 58429215 | 58442218 | 0.001896858 | −32.34920635 | Hypo-methylated | PCa\|NT DMRs |
| chr11 | 58940671 | 58940913 | 0.004095673 | 45.96507937 | Hyper-methylated | PCa\|NT DMRs |
| chr11 | 59221275 | 59278014 | 1.42E−05 | −35.71142857 | Hypo-methylated | PCa\|NT DMRs |
| chr11 | 59323338 | 59324098 | 0.001674516 | 21.28515406 | Hyper-methylated | PCa\|NT DMRs |
| chr11 | 59333340 | 59333864 | 4.96E−09 | 24.12315271 | Hyper-methylated | PCa\|NT DMRs |
| chr11 | 59478100 | 59520668 | 0.000205174 | −30.61785714 | Hypo-methylated | PCa\|NT DMRs |
| chr11 | 59968481 | 60105291 | 1.84E−08 | −28.66897547 | Hypo-methylated | PCa\|NT DMRs |
| chr11 | 60534949 | 60535138 | 0.008897139 | 23.62337662 | Hyper-methylated | PCa\|NT DMRs |
| chr11 | 60778547 | 60779705 | 0.002433478 | 39.3077381 | Hyper-methylated | PCa\|NT DMRs |
| chr11 | 60869969 | 60887659 | 0.001083007 | 23.42142857 | Hyper-methylated | PCa\|NT DMRs |
| chr11 | 61049111 | 61049231 | 0.003689112 | −21.97362637 | Hypo-methylated | PCa\|NT DMRs |
| chr11 | 61109959 | 61110130 | 0.00641359 | 35.83333333 | Hyper-methylated | PCa\|NT DMRs |
| chr11 | 61256187 | 61257657 | 0.00064571 | −50.74829932 | Hypo-methylated | PCa\|NT DMRs |
| chr11 | 61343520 | 61344277 | 0.000137816 | −53.59325397 | Hypo-methylated | PCa\|NT DMRs |
| chr11 | 61375495 | 61378730 | 0.00064571 | −40.92176871 | Hypo-methylated | PCa\|NT DMRs |
| chr11 | 61394328 | 61394537 | 0.005968871 | −41.46190476 | Hypo-methylated | PCa\|NT DMRs |
| chr11 | 61451512 | 61452306 | 0.00064571 | 44.40816327 | Hyper-methylated | PCa\|NT DMRs |
| chr11 | 61593787 | 61595285 | 0.004677148 | 26.17359307 | Hyper-methylated | PCa\|NT DMRs |
| chr11 | 61596960 | 61600845 | 0.002802605 | −28.63265306 | Hypo-methylated | PCa\|NT DMRs |
| chr11 | 61671833 | 61672078 | 0.005968871 | 46.6047619 | Hyper-methylated | PCa\|NT DMRs |
| chr11 | 62273922 | 62277677 | 0.005968871 | −23.46137566 | Hypo-methylated | PCa\|NT DMRs |
| chr11 | 62380679 | 62381007 | 0.005438982 | −32.15714286 | Hypo-methylated | PCa\|NT DMRs |
| chr11 | 62447157 | 62449378 | 0.000205174 | −32.23511905 | Hypo-methylated | PCa\|NT DMRs |
| chr11 | 62555857 | 62558534 | 0.001013662 | −25.80748299 | Hypo-methylated | PCa\|NT DMRs |
| chr11 | 62609849 | 62610107 | 0.000209486 | 29.82603175 | Hyper-methylated | PCa\|NT DMRs |
| chr11 | 62673739 | 62677297 | 0.000150833 | −30.52933333 | Hypo-methylated | PCa\|NT DMRs |
| chr11 | 62677889 | 62678152 | 6.42E−05 | −26.6005291 | Hypo-methylated | PCa\|NT DMRs |
| chr11 | 62689065 | 62689123 | 0.008482405 | −37.97142857 | Hypo-methylated | PCa\|NT DMRs |
| chr11 | 63257599 | 63278917 | 1.53E−05 | 31.75038402 | Hyper-methylated | PCa\|NT DMRs |
| chr11 | 63529502 | 63531940 | 7.77E−35 | −22.27085485 | Hypo-methylated | PCa\|NT DMRs |
| chr11 | 63608634 | 63609930 | 0.001969641 | −50.69047619 | Hypo-methylated | PCa\|NT DMRs |
| chr11 | 63766316 | 63766738 | 1.02E−05 | 32.27928571 | Hyper-methylated | PCa\|NT DMRs |
| chr11 | 63768254 | 63775073 | 3.87E−06 | 23.51582418 | Hyper-methylated | PCa\|NT DMRs |
| chr11 | 63802060 | 63802461 | 0.003448944 | 30.15306122 | Hyper-methylated | PCa\|NT DMRs |
| chr11 | 63913750 | 63913964 | 0.002437742 | 29.96031746 | Hyper-methylated | PCa\|NT DMRs |
| chr11 | 63932230 | 63932757 | 0.000205174 | −43.78571429 | Hypo-methylated | PCa\|NT DMRs |
| chr11 | 63972859 | 63978501 | 8.22E−09 | 34.14940476 | Hyper-methylated | PCa\|NT DMRs |
| chr11 | 64069092 | 64069339 | 0.00569382 | −22.89285714 | Hypo-methylated | PCa\|NT DMRs |
| chr11 | 64074084 | 64074166 | 0.000205174 | 35.61488095 | Hyper-methylated | PCa\|NT DMRs |
| chr11 | 64162954 | 64167498 | 0.000191452 | 32.52380952 | Hyper-methylated | PCa\|NT DMRs |
| chr11 | 64177216 | 64177389 | 0.005968871 | 39.68571429 | Hyper-methylated | PCa\|NT DMRs |
| chr11 | 64375615 | 64375708 | 0.001969641 | −54.82585034 | Hypo-methylated | PCa\|NT DMRs |
| chr11 | 64478990 | 64479058 | 0.00064571 | 36.39047619 | Hyper-methylated | PCa\|NT DMRs |
| chr11 | 64480312 | 64480500 | 4.20E−06 | 22.25396825 | Hyper-methylated | PCa\|NT DMRs |
| chr11 | 64609864 | 64611748 | 0.000357945 | −36.87751323 | Hypo-methylated | PCa\|NT DMRs |
| chr11 | 64618782 | 64630585 | 8.36E−09 | 35.07356657 | Hyper-methylated | PCa\|NT DMRs |
| chr11 | 64632705 | 64643976 | 1.94E−10 | 27.76675325 | Hyper-methylated | PCa\|NT DMRs |
| chr11 | 64659028 | 64659131 | 0.001181579 | −29.93877551 | Hypo-methylated | PCa\|NT DMRs |
| chr11 | 64660850 | 64662239 | 0.004505524 | 28.50992063 | Hyper-methylated | PCa\|NT DMRs |
| chr11 | 64706819 | 64721931 | 0.006589535 | −22.43095238 | Hypo-methylated | PCa\|NT DMRs |
| chr11 | 64757720 | 64758048 | 0.00064571 | 39.58367347 | Hyper-methylated | PCa\|NT DMRs |
| chr11 | 65006862 | 65010385 | 0.002534928 | −24.63 | Hypo-methylated | PCa\|NT DMRs |
| chr11 | 65145930 | 65146458 | 0.007959104 | 36.91020408 | Hyper-methylated | PCa\|NT DMRs |
| chr11 | 65307124 | 65307409 | 1.89E−05 | −24.24920635 | Hypo-methylated | PCa\|NT DMRs |
| chr11 | 65414273 | 65415386 | 1.03E−17 | −47.82563025 | Hypo-methylated | PCa\|NT DMRs |
| chr11 | 65459968 | 65460448 | 0.003448944 | −36.27891156 | Hypo-methylated | PCa\|NT DMRs |
| chr11 | 65549119 | 65549225 | 0.006589535 | 22.51428571 | Hyper-methylated | PCa\|NT DMRs |
| chr11 | 65553636 | 65553876 | 0.00020505 | 25.20017637 | Hyper-methylated | PCa\|NT DMRs |
| chr11 | 65560596 | 65560988 | 0.000111427 | −39.68238095 | Hypo-methylated | PCa\|NT DMRs |
| chr11 | 65658854 | 65667189 | 8.33E−17 | 24.46147959 | Hyper-methylated | PCa\|NT DMRs |
| chr11 | 65707163 | 65727355 | 1.35E−10 | −22.94155844 | Hypo-methylated | PCa\|NT DMRs |
| chr11 | 65790280 | 65790721 | 0.009165255 | −23.08280423 | Hypo-methylated | PCa\|NT DMRs |
| chr11 | 66084306 | 66084895 | 3.51E−06 | 23.40119048 | Hyper-methylated | PCa\|NT DMRs |
| chr11 | 66179672 | 66179920 | 0.001896858 | −37.58730159 | Hypo-methylated | PCa\|NT DMRs |
| chr11 | 66326554 | 66327126 | 0.000157858 | 20.08050595 | Hyper-methylated | PCa\|NT DMRs |
| chr11 | 66484890 | 66486141 | 3.90E−08 | −50.18178054 | Hypo-methylated | PCa\|NT DMRs |
| chr11 | 66816488 | 66816867 | 0.00641359 | 29.3968254 | Hyper-methylated | PCa\|NT DMRs |
| chr11 | 67043561 | 67046287 | 0.000226671 | 30.1377425 | Hyper-methylated | PCa\|NT DMRs |
| chr11 | 67049199 | 67054945 | 7.34E−12 | 30.2312256 | Hyper-methylated | PCa\|NT DMRs |
| chr11 | 67164636 | 67164789 | 0.00064571 | 47.26870748 | Hyper-methylated | PCa\|NT DMRs |
| chr11 | 67171015 | 67173367 | 6.94E−06 | 34.78027211 | Hyper-methylated | PCa\|NT DMRs |
| chr11 | 67176301 | 67177283 | 2.72E−07 | 33.35714286 | Hyper-methylated | PCa\|NT DMRs |
| chr11 | 67178868 | 67179368 | 0.000320751 | −20.01587302 | Hypo-methylated | PCa\|NT DMRs |
| chr11 | 67204882 | 67206343 | 0.000728857 | 33.8707483 | Hyper-methylated | PCa\|NT DMRs |
| chr11 | 67210077 | 67210845 | 2.98E−06 | −36.78280423 | Hypo-methylated | PCa\|NT DMRs |
| chr11 | 67219498 | 67220021 | 0.000145907 | −38.95142857 | Hypo-methylated | PCa\|NT DMRs |
| chr11 | 67345075 | 67349375 | 0.004535472 | −31.39821429 | Hypo-methylated | PCa\|NT DMRs |
| chr11 | 67733489 | 67746317 | 0.001139334 | −37.53809524 | Hypo-methylated | PCa\|NT DMRs |
| chr11 | 67770789 | 67770905 | 5.52E−05 | 31.51948052 | Hyper-methylated | PCa\|NT DMRs |
| chr11 | 67778480 | 67778837 | 1.76E−06 | 44.68809524 | Hyper-methylated | PCa\|NT DMRs |
| chr11 | 67786698 | 67788233 | 0.007679542 | −33.98677249 | Hypo-methylated | PCa\|NT DMRs |
| chr11 | 67803806 | 67804652 | 0.000100211 | 37.76666667 | Hyper-methylated | PCa\|NT DMRs |
| chr11 | 68049738 | 68050189 | 0.00064571 | 50.56802721 | Hyper-methylated | PCa\|NT DMRs |
| chr11 | 68064507 | 68064952 | 5.42E−05 | −33.91468254 | Hypo-methylated | PCa\|NT DMRs |
| chr11 | 68065644 | 68066064 | 0.001789867 | −35.15925926 | Hypo-methylated | PCa\|NT DMRs |
| chr11 | 68066301 | 68076037 | 0.000611234 | 24.33746032 | Hyper-methylated | PCa\|NT DMRs |
| chr11 | 68098668 | 68098999 | 0.000122432 | 40.73593074 | Hyper-methylated | PCa\|NT DMRs |
| chr11 | 68173861 | 68176237 | 0.000205174 | −53.81011905 | Hypo-methylated | PCa\|NT DMRs |
| chr11 | 68207094 | 68207415 | 0.000424462 | −23.92517007 | Hypo-methylated | PCa\|NT DMRs |
| chr11 | 68419149 | 68419629 | 0.00170819 | −31.21428571 | Hypo-methylated | PCa\|NT DMRs |
| chr11 | 68452595 | 68452685 | 2.18E−05 | 24.58319328 | Hyper-methylated | PCa\|NT DMRs |
| chr11 | 68530104 | 68532038 | 0.000339757 | 26.19345238 | Hyper-methylated | PCa\|NT DMRs |
| chr11 | 68534208 | 68534505 | 0.00064571 | 39.08163265 | Hyper-methylated | PCa\|NT DMRs |
| chr11 | 68592697 | 68597846 | 0.002931637 | 28.44057143 | Hyper-methylated | PCa\|NT DMRs |
| chr11 | 68621969 | 68627789 | 2.62E−09 | 26.04550265 | Hyper-methylated | PCa\|NT DMRs |
| chr11 | 68637018 | 68638340 | 1.07E−06 | 31.25192744 | Hyper-methylated | PCa\|NT DMRs |
| chr11 | 68639648 | 68643065 | 0.000205174 | −38.37797619 | Hypo-methylated | PCa\|NT DMRs |
| chr11 | 68701326 | 68702292 | 0.00064571 | −21.29931973 | Hypo-methylated | PCa\|NT DMRs |
| chr11 | 68864677 | 68867966 | 0.002675664 | 23.68696742 | Hyper-methylated | PCa\|NT DMRs |
| chr11 | 68890034 | 68890407 | 0.002641501 | −25.69047619 | Hypo-methylated | PCa\|NT DMRs |
| chr11 | 68924647 | 68925108 | 7.75E−10 | 22.57777778 | Hyper-methylated | PCa\|NT DMRs |
| chr11 | 68963501 | 68963776 | 0.000112123 | −25.93253968 | Hypo-methylated | PCa\|NT DMRs |
| chr11 | 69019266 | 69024680 | 0.000822388 | −27.11688312 | Hypo-methylated | PCa\|NT DMRs |
| chr11 | 69045416 | 69045825 | 0.001969641 | −46.81088435 | Hypo-methylated | PCa\|NT DMRs |
| chr11 | 69189180 | 69189636 | 6.21E−05 | 37.60714286 | Hyper-methylated | PCa\|NT DMRs |
| chr11 | 69193134 | 69193688 | 0.000457395 | −27.12527473 | Hypo-methylated | PCa\|NT DMRs |
| chr11 | 69229729 | 69230044 | 0.001969641 | 35.5952381 | Hyper-methylated | PCa\|NT DMRs |
| chr11 | 69258282 | 69258722 | 8.37E−10 | 32.54530612 | Hyper-methylated | PCa\|NT DMRs |
| chr11 | 69276968 | 69280578 | 9.48E−07 | −26.25192012 | Hypo-methylated | PCa\|NT DMRs |
| chr11 | 69285805 | 69286585 | 0.000186814 | 23.43142857 | Hyper-methylated | PCa\|NT DMRs |
| chr11 | 69311990 | 69312363 | 1.76E−06 | −27.34798535 | Hypo-methylated | PCa\|NT DMRs |
| chr11 | 69339369 | 69339606 | 0.005513661 | −29.86666667 | Hypo-methylated | PCa\|NT DMRs |
| chr11 | 69458552 | 69461852 | 0.000549692 | −21.71390977 | Hypo-methylated | PCa\|NT DMRs |
| chr11 | 69465895 | 69470244 | 5.64E−06 | −38.13984962 | Hypo-methylated | PCa\|NT DMRs |
| chr11 | 69520931 | 69527291 | 3.67E−06 | 27.4247619 | Hyper-methylated | PCa\|NT DMRs |
| chr11 | 69677205 | 69677632 | 0.000117699 | 23.07744361 | Hyper-methylated | PCa\|NT DMRs |
| chr11 | 69683075 | 69683462 | 5.18E−06 | 26.80910973 | Hyper-methylated | PCa\|NT DMRs |
| chr11 | 69707569 | 69707837 | 0.005968871 | 48.98761905 | Hyper-methylated | PCa\|NT DMRs |
| chr11 | 69832153 | 69832553 | 5.30E−09 | 20.07053571 | Hyper-methylated | PCa\|NT DMRs |
| chr11 | 69949556 | 69950083 | 0.00068166 | −35.39880952 | Hypo-methylated | PCa\|NT DMRs |
| chr11 | 70043914 | 70048550 | 0.005968871 | −21.88359788 | Hypo-methylated | PCa\|NT DMRs |
| chr11 | 70261857 | 70261911 | 0.00023371 | 55.32619048 | Hyper-methylated | PCa\|NT DMRs |
| chr11 | 70278186 | 70278327 | 0.009924186 | 35.44973545 | Hyper-methylated | PCa\|NT DMRs |
| chr11 | 70282413 | 70283318 | 7.27E−06 | −36.51407867 | Hypo-methylated | PCa\|NT DMRs |
| chr11 | 70713938 | 70714173 | 0.000118733 | −23.89365079 | Hypo-methylated | PCa\|NT DMRs |
| chr11 | 71189249 | 71191381 | 0.007239577 | 21.99365079 | Hyper-methylated | PCa\|NT DMRs |
| chr11 | 71326126 | 71348524 | 9.46E−05 | −33.13378685 | Hypo-methylated | PCa\|NT DMRs |
| chr11 | 71792288 | 71792393 | 0.00064571 | −29.05714286 | Hypo-methylated | PCa\|NT DMRs |
| chr11 | 72328987 | 72330016 | 0.000377317 | −21.1577381 | Hypo-methylated | PCa\|NT DMRs |
| chr11 | 72336639 | 72337206 | 0.001443483 | −25.88004535 | Hypo-methylated | PCa\|NT DMRs |
| chr11 | 72527551 | 72528765 | 0.002097193 | 48.61292517 | Hyper-methylated | PCa\|NT DMRs |
| chr11 | 72532653 | 72533479 | 5.58E−16 | −37.86146384 | Hypo-methylated | PCa\|NT DMRs |
| chr11 | 72915848 | 72917050 | 6.42E−05 | 51.07407407 | Hyper-methylated | PCa\|NT DMRs |
| chr11 | 72954276 | 72955231 | 8.96E−05 | −34.32420635 | Hypo-methylated | PCa\|NT DMRs |
| chr11 | 72980867 | 72986022 | 0.005896218 | 20.13508772 | Hyper-methylated | PCa\|NT DMRs |
| chr11 | 73372519 | 73372610 | 0.00025637 | 25.3281746 | Hyper-methylated | PCa\|NT DMRs |
| chr11 | 73668519 | 73668840 | 0.000196301 | 22.63015873 | Hyper-methylated | PCa\|NT DMRs |
| chr11 | 74178026 | 74179320 | 5.03E−08 | 26.42772109 | Hyper-methylated | PCa\|NT DMRs |
| chr11 | 75139269 | 75140869 | 4.23E−09 | 23.75014006 | Hyper-methylated | PCa\|NT DMRs |
| chr11 | 75184864 | 75188249 | 0.005574525 | −40.15930736 | Hypo-methylated | PCa\|NT DMRs |
| chr11 | 75433466 | 75433747 | 0.005968871 | −50.62857143 | Hypo-methylated | PCa\|NT DMRs |
| chr11 | 75852175 | 75852432 | 0.007959104 | −24.55102041 | Hypo-methylated | PCa\|NT DMRs |
| chr11 | 76371565 | 76372315 | 0.00064571 | −34.02380952 | Hypo-methylated | PCa\|NT DMRs |
| chr11 | 76375895 | 76377681 | 0.000233379 | 40.86849817 | Hyper-methylated | PCa\|NT DMRs |
| chr11 | 76397539 | 76397753 | 0.00064571 | 53.91836735 | Hyper-methylated | PCa\|NT DMRs |
| chr11 | 76430376 | 76430585 | 6.69E−05 | −22.68571429 | Hypo-methylated | PCa\|NT DMRs |
| chr11 | 76858730 | 76859141 | 3.16E−06 | 38.00634921 | Hyper-methylated | PCa\|NT DMRs |
| chr11 | 77907224 | 77907379 | 2.74E−05 | 21.99548872 | Hyper-methylated | PCa\|NT DMRs |
| chr11 | 78614210 | 78614533 | 9.87E−08 | 28.75555556 | Hyper-methylated | PCa\|NT DMRs |
| chr11 | 82815293 | 82815506 | 0.004057568 | −41.81746032 | Hypo-methylated | PCa\|NT DMRs |
| chr11 | 85521574 | 85521781 | 0.009924186 | 23.12698413 | Hyper-methylated | PCa\|NT DMRs |
| chr11 | 85913749 | 85913798 | 0.005968871 | 39.65714286 | Hyper-methylated | PCa\|NT DMRs |
| chr11 | 86026752 | 86087804 | 0.006882266 | 21.85513784 | Hyper-methylated | PCa\|NT DMRs |
| chr11 | 87100459 | 87845275 | 1.02E−07 | −36.34900794 | Hypo-methylated | PCa\|NT DMRs |
| chr11 | 87908783 | 87908950 | 5.19E−07 | 20.76190476 | Hyper-methylated | PCa\|NT DMRs |
| chr11 | 88149982 | 88235508 | 0.00064571 | −21.33386243 | Hypo-methylated | PCa\|NT DMRs |
| chr11 | 88713712 | 89135566 | 6.76E−07 | −28.23308271 | Hypo-methylated | PCa\|NT DMRs |
| chr11 | 89224506 | 89225015 | 1.92E−05 | 23.48370927 | Hyper-methylated | PCa\|NT DMRs |
| chr11 | 89983223 | 90716615 | 3.06E−16 | −27.08809524 | Hypo-methylated | PCa\|NT DMRs |
| chr11 | 90800075 | 91145307 | 4.38E−05 | −26.22202381 | Hypo-methylated | PCa\|NT DMRs |
| chr11 | 91549570 | 91805803 | 2.65E−09 | −34.98285714 | Hypo-methylated | PCa\|NT DMRs |
| chr11 | 91902869 | 91946834 | 0.000333533 | −29.63027211 | Hypo-methylated | PCa\|NT DMRs |
| chr11 | 93680657 | 93707470 | 0.000294557 | −24.71688312 | Hypo-methylated | PCa\|NT DMRs |
| chr11 | 93964904 | 94129108 | 5.78E−09 | −25.65733333 | Hypo-methylated | PCa\|NT DMRs |
| chr11 | 94319404 | 94333770 | 0.003809353 | −27.33190476 | Hypo-methylated | PCa\|NT DMRs |
| chr11 | 94840950 | 94841211 | 1.42E−05 | −31.26530612 | Hypo-methylated | PCa\|NT DMRs |
| chr11 | 96074857 | 96075066 | 2.24E−06 | 23.85854342 | Hyper-methylated | PCa\|NT DMRs |
| chr11 | 96792815 | 97538401 | 6.95E−19 | −30.89047619 | Hypo-methylated | PCa\|NT DMRs |
| chr11 | 97663020 | 97695853 | 0.005968871 | −30.34285714 | Hypo-methylated | PCa\|NT DMRs |
| chr11 | 98078394 | 98294685 | 5.19E−17 | −30.17584416 | Hypo-methylated | PCa\|NT DMRs |
| chr11 | 98341630 | 98685433 | 1.64E−20 | −28.06955064 | Hypo-methylated | PCa\|NT DMRs |
| chr11 | 99013106 | 99279937 | 2.42E−08 | −34.26845238 | Hypo-methylated | PCa\|NT DMRs |
| chr11 | 100146971 | 100207638 | 0.001969641 | −29.86111111 | Hypo-methylated | PCa\|NT DMRs |
| chr11 | 100359495 | 100387851 | 0.000377317 | −33.97380952 | Hypo-methylated | PCa\|NT DMRs |
| chr11 | 100940788 | 100941020 | 0.000118733 | −24.65079365 | Hypo-methylated | PCa\|NT DMRs |
| chr11 | 101112312 | 101437496 | 8.48E−07 | −22.31493506 | Hypo-methylated | PCa\|NT DMRs |
| chr11 | 101558336 | 101673496 | 0.005968871 | −33.05714286 | Hypo-methylated | PCa\|NT DMRs |
| chr11 | 103497084 | 104031760 | 2.12E−17 | −35.38873626 | Hypo-methylated | PCa\|NT DMRs |
| chr11 | 105716330 | 105735453 | 0.000370938 | −28.98268398 | Hypo-methylated | PCa\|NT DMRs |
| chr11 | 108389463 | 108390791 | 0.007959104 | −24.7952381 | Hypo-methylated | PCa\|NT DMRs |
| chr11 | 108873635 | 109279296 | 4.36E−12 | −40.08857143 | Hypo-methylated | PCa\|NT DMRs |
| chr11 | 109396626 | 109496463 | 8.34E−06 | −38.78285714 | Hypo-methylated | PCa\|NT DMRs |
| chr11 | 109507475 | 109931607 | 4.80E−10 | −35.96978654 | Hypo-methylated | PCa\|NT DMRs |
| chr11 | 111154952 | 111155121 | 1.69E−06 | 27.54061625 | Hyper-methylated | PCa\|NT DMRs |
| chr11 | 111228370 | 111250224 | 4.39E−07 | 36.79873016 | Hyper-methylated | PCa\|NT DMRs |
| chr11 | 112617155 | 112705405 | 0.000163639 | −22.57806122 | Hypo-methylated | PCa\|NT DMRs |
| chr11 | 113107412 | 113108244 | 0.005968871 | −20.66285714 | Hypo-methylated | PCa\|NT DMRs |
| chill | 113846762 | 113847036 | 0.005531225 | 31.61269841 | Hyper-methylated | PCa\|NT DMRs |
| chr11 | 113907106 | 113911682 | 0.001965153 | −22.64835165 | Hypo-methylated | PCa\|NT DMRs |
| chr11 | 114481872 | 114483968 | 3.64E−09 | −27.20252101 | Hypo-methylated | PCa\|NT DMRs |
| chr11 | 114760625 | 114835973 | 0.000787744 | −26.25634921 | Hypo-methylated | PCa\|NT DMRs |
| chr11 | 114920485 | 114920802 | 0.00064571 | −27.61904762 | Hypo-methylated | PCa\|NT DMRs |
| chr11 | 116399323 | 116438364 | 0.006257887 | −21.85693582 | Hypo-methylated | PCa\|NT DMRs |
| chr11 | 116474707 | 116498296 | 3.36E−06 | −23.76190476 | Hypo-methylated | PCa\|NT DMRs |
| chr11 | 117373488 | 117373667 | 0.00641359 | −32.36507937 | Hypo-methylated | PCa\|NT DMRs |
| chr11 | 117459220 | 117462571 | 1.05E−06 | −26.89880952 | Hypo-methylated | PCa\|NT DMRs |
| chr11 | 117475269 | 117495957 | 0.005513661 | −33.97278912 | Hypo-methylated | PCa\|NT DMRs |
| chr11 | 117534685 | 117534947 | 0.007560755 | 24.57142857 | Hyper-methylated | PCa\|NT DMRs |
| chr11 | 117588832 | 117589012 | 0.000974795 | −51.65357143 | Hypo-methylated | PCa\|NT DMRs |
| chr11 | 117652762 | 117665030 | 5.99E−05 | −20.62637363 | Hypo-methylated | PCa\|NT DMRs |
| chr11 | 117694283 | 117699419 | 2.19E−08 | 31.86058201 | Hyper-methylated | PCa\|NT DMRs |
| chr11 | 118003671 | 118003733 | 0.001969641 | −37.04761905 | Hypo-methylated | PCa\|NT DMRs |
| chr11 | 118051171 | 118052222 | 0.000118733 | 40.18624339 | Hyper-methylated | PCa\|NT DMRs |
| chr11 | 118505570 | 118506210 | 0.002842594 | −25.41980952 | Hypo-methylated | PCa\|NT DMRs |
| chr11 | 118743125 | 118750827 | 1.42E−05 | 22.5978836 | Hyper-methylated | PCa\|NT DMRs |
| chr11 | 118788503 | 118794718 | 0.000313712 | −20.85102041 | Hypo-methylated | PCa\|NT DMRs |
| chr11 | 118986793 | 118987127 | 0.002087251 | 32.71428571 | Hyper-methylated | PCa\|NT DMRs |
| chr11 | 119188026 | 119188299 | 0.003391914 | 22.41269841 | Hyper-methylated | PCa\|NT DMRs |
| chr11 | 119206378 | 119206667 | 0.004236974 | −28.05904762 | Hypo-methylated | PCa\|NT DMRs |
| chr11 | 119211611 | 119214288 | 0.005513661 | 30.14642857 | Hyper-methylated | PCa\|NT DMRs |
| chr11 | 119251680 | 119252044 | 0.00023371 | 28.02597403 | Hyper-methylated | PCa\|NT DMRs |
| chr11 | 119580056 | 119580589 | 0.002994094 | 35.59307359 | Hyper-methylated | PCa\|NT DMRs |
| chr11 | 119600926 | 119618861 | 7.69E−05 | 21.91456583 | Hyper-methylated | PCa\|NT DMRs |
| chr11 | 120063694 | 120074851 | 0.002647964 | −29.88435374 | Hypo-methylated | PCa\|NT DMRs |
| chr11 | 120082136 | 120082953 | 1.46E−05 | 21.53030303 | Hyper-methylated | PCa\|NT DMRs |
| chr11 | 120093121 | 120096386 | 6.81E−05 | 35.98571429 | Hyper-methylated | PCa\|NT DMRs |
| chr11 | 120384475 | 120387576 | 0.000241095 | 22.97994987 | Hyper-methylated | PCa\|NT DMRs |
| chr11 | 120387686 | 120388052 | 2.05E−05 | −30.1462585 | Hypo-methylated | PCa\|NT DMRs |
| chr11 | 120434823 | 120435293 | 8.63E−08 | 21.55299539 | Hyper-methylated | PCa\|NT DMRs |
| chr11 | 120837830 | 120837916 | 0.005968871 | −44.57142857 | Hypo-methylated | PCa\|NT DMRs |
| chr11 | 121107588 | 121107707 | 0.003871035 | −23.33928571 | Hypo-methylated | PCa\|NT DMRs |
| chr11 | 121349767 | 121349947 | 0.005968871 | 38.02857143 | Hyper-methylated | PCa\|NT DMRs |
| chr11 | 121533331 | 121565075 | 4.57E−08 | −36.64494048 | Hypo-methylated | PCa\|NT DMRs |
| chr11 | 122873060 | 122900389 | 5.05E−06 | −21.92142857 | Hypo-methylated | PCa\|NT DMRs |
| chr11 | 122972110 | 123016468 | 6.42E−05 | −23.76666667 | Hypo-methylated | PCa\|NT DMRs |
| chr11 | 123461633 | 123507562 | 6.90E−06 | −32.29904762 | Hypo-methylated | PCa\|NT DMRs |
| chr11 | 123651223 | 123928401 | 1.55E−06 | −34.8375 | Hypo-methylated | PCa\|NT DMRs |
| chr11 | 124284677 | 124311424 | 0.001969641 | −24.05380952 | Hypo-methylated | PCa\|NT DMRs |
| chr11 | 124405040 | 124435266 | 0.00064571 | −31.62433862 | Hypo-methylated | PCa\|NT DMRs |
| chr11 | 124628054 | 124631667 | 3.75E−06 | 25.27165899 | Hyper-methylated | PCa\|NT DMRs |
| chr11 | 124715092 | 124716382 | 0.00641359 | 23.82380952 | Hyper-methylated | PCa\|NT DMRs |
| chr11 | 124747040 | 124748116 | 0.002111644 | 29.05091575 | Hyper-methylated | PCa\|NT DMRs |
| chr11 | 125743714 | 125753502 | 0.001969641 | −20.95238095 | Hypo-methylated | PCa\|NT DMRs |
| chr11 | 125774146 | 125774493 | 4.48E−08 | 22.19847619 | Hyper-methylated | PCa\|NT DMRs |
| chr11 | 125822831 | 125823311 | 0.004095673 | −48.07936508 | Hypo-methylated | PCa\|NT DMRs |
| chr11 | 125985045 | 125986371 | 0.000914361 | −37.37585034 | Hypo-methylated | PCa\|NT DMRs |
| chr11 | 126206659 | 126215206 | 0.007165874 | 20.03119048 | Hyper-methylated | PCa\|NT DMRs |
| chr11 | 126316743 | 126317382 | 7.18E−06 | −20.41111111 | Hypo-methylated | PCa\|NT DMRs |
| chr11 | 126345215 | 126345532 | 0.000196301 | 36.01388889 | Hyper-methylated | PCa\|NT DMRs |
| chr11 | 126381434 | 126390974 | 2.36E−07 | −28.14729064 | Hypo-methylated | PCa\|NT DMRs |
| chr11 | 126455286 | 126485817 | 4.34E−07 | −33.08095238 | Hypo-methylated | PCa\|NT DMRs |
| chr11 | 126881639 | 126987170 | 0.000270512 | −25.78403361 | Hypo-methylated | PCa\|NT DMRs |
| chr11 | 127012786 | 127239512 | 5.68E−12 | −29.69825784 | Hypo-methylated | PCa\|NT DMRs |
| chr11 | 127282817 | 127549907 | 4.85E−06 | −31.00702948 | Hypo-methylated | PCa\|NT DMRs |
| chr11 | 127624031 | 128103561 | 3.06E−16 | −27.0217484 | Hypo-methylated | PCa\|NT DMRs |
| chr11 | 128205794 | 128206332 | 0.000215808 | −21.7037037 | Hypo-methylated | PCa\|NT DMRs |
| chr11 | 128685176 | 128693881 | 3.82E−05 | −25.09747899 | Hypo-methylated | PCa\|NT DMRs |
| chr11 | 128734754 | 128751765 | 0.002437742 | −37.14497354 | Hypo-methylated | PCa\|NT DMRs |
| chr11 | 130185670 | 130185785 | 0.001969641 | 54.56349206 | Hyper-methylated | PCa\|NT DMRs |
| chr11 | 130271080 | 130271852 | 0.003622801 | 43.16666667 | Hyper-methylated | PCa\|NT DMRs |
| chr11 | 131455241 | 131455478 | 0.003290817 | 32.25 | Hyper-methylated | PCa\|NT DMRs |
| chr11 | 132204883 | 132252514 | 1.37E−09 | −31.74091711 | Hypo-methylated | PCa\|NT DMRs |
| chr11 | 132315341 | 132558998 | 1.63E−11 | −30.63465608 | Hypo-methylated | PCa\|NT DMRs |
| chr11 | 132830113 | 132932992 | 0.000314331 | −22.45813492 | Hypo-methylated | PCa\|NT DMRs |
| chr11 | 132945383 | 132950104 | 5.87E−05 | −28.7110119 | Hypo-methylated | PCa\|NT DMRs |
| chr11 | 133023166 | 133032523 | 4.41E−05 | −21.63151927 | Hypo-methylated | PCa\|NT DMRs |
| chr11 | 133155303 | 133209529 | 0.002437742 | −28.46031746 | Hypo-methylated | PCa\|NT DMRs |
| chr11 | 133252438 | 133256936 | 0.009106413 | −36.07380952 | Hypo-methylated | PCa\|NT DMRs |
| chr11 | 133308628 | 133386584 | 8.31E−07 | −30.09724311 | Hypo-methylated | PCa\|NT DMRs |
| chr11 | 133433718 | 133653323 | 5.49E−14 | −30.56162047 | Hypo-methylated | PCa\|NT DMRs |
| chr11 | 133682204 | 133701882 | 0.001336455 | −30.33928571 | Hypo-methylated | PCa\|NT DMRs |
| chr11 | 133788094 | 133789240 | 1.87E−06 | 26.14646465 | Hyper-methylated | PCa\|NT DMRs |
| chr11 | 133789404 | 133789898 | 1.14E−07 | 21.66522367 | Hyper-methylated | PCa\|NT DMRs |
| chr11 | 133798159 | 133798330 | 0.004350823 | −27.63214286 | Hypo-methylated | PCa\|NT DMRs |
| chr11 | 133821373 | 133821967 | 2.12E−05 | −24.6468254 | Hypo-methylated | PCa\|NT DMRs |
| chr11 | 133841580 | 133848082 | 4.09E−06 | −27.1115942 | Hypo-methylated | PCa\|NT DMRs |
| chr11 | 133906819 | 133907005 | 0.000214406 | 32.23076923 | Hyper-methylated | PCa\|NT DMRs |
| chr11 | 133912462 | 133914968 | 0.000118733 | −25.57142857 | Hypo-methylated | PCa\|NT DMRs |
| chr11 | 133917160 | 133920309 | 4.62E−05 | −38.92380952 | Hypo-methylated | PCa\|NT DMRs |
| chr11 | 133939628 | 133994763 | 0.001764697 | −22.58894009 | Hypo-methylated | PCa\|NT DMRs |
| chr11 | 134009230 | 134011323 | 0.006589535 | −38.02904762 | Hypo-methylated | PCa\|NT DMRs |
| chr11 | 134405717 | 134406735 | 0.000269433 | 40.62755102 | Hyper-methylated | PCa\|NT DMRs |
| chr11 | 134526897 | 134527288 | 6.50E−07 | −27.13142857 | Hypo-methylated | PCa\|NT DMRs |
| chr11 | 134670498 | 134717506 | 1.74E−06 | −21.53794643 | Hypo-methylated | PCa\|NT DMRs |
| chr11 | 134854446 | 134919563 | 1.05E−07 | −21.75687831 | Hypo-methylated | PCa\|NT DMRs |
| chr12 | 569808 | 570443 | 7.05E−12 | 25.60952381 | Hyper-methylated | PCa\|NT DMRs |
| chr12 | 655964 | 662051 | 0.002400984 | −27.98095238 | Hypo-methylated | PCa\|NT DMRs |
| chr12 | 671272 | 671670 | 0.00023371 | 44.31714286 | Hyper-methylated | PCa\|NT DMRs |
| chr12 | 679463 | 681562 | 0.005669451 | −35.80532213 | Hypo-methylated | PCa\|NT DMRs |
| chr12 | 752244 | 753200 | 3.19E−06 | 22.85281385 | Hyper-methylated | PCa\|NT DMRs |
| chr12 | 1025178 | 1025633 | 4.54E−06 | 35.5877193 | Hyper-methylated | PCa\|NT DMRs |
| chr12 | 1582667 | 1582873 | 0.001969641 | 21.19444444 | Hyper-methylated | PCa\|NT DMRs |
| chr12 | 1605430 | 1605951 | 0.001969641 | −50.22460317 | Hypo-methylated | PCa\|NT DMRs |
| chr12 | 1638997 | 1639209 | 2.24E−05 | 20.96357143 | Hyper-methylated | PCa\|NT DMRs |
| chr12 | 1640961 | 1646739 | 0.000632596 | 30.02424242 | Hyper-methylated | PCa\|NT DMRs |
| chr12 | 1740149 | 1740637 | 1.52E−07 | 47.83163265 | Hyper-methylated | PCa\|NT DMRs |
| chr12 | 1744770 | 1744965 | 0.005968871 | 48.71428571 | Hyper-methylated | PCa\|NT DMRs |
| chr12 | 1756030 | 1756261 | 0.003290817 | 39.08035714 | Hyper-methylated | PCa\|NT DMRs |
| chr12 | 1770630 | 1770881 | 0.000879355 | 22.00300752 | Hyper-methylated | PCa\|NT DMRs |
| chr12 | 1906018 | 1907220 | 3.17E−06 | 23.7182684 | Hyper-methylated | PCa\|NT DMRs |
| chr12 | 1952172 | 1954957 | 0.000145094 | −24.1765873 | Hypo-methylated | PCa\|NT DMRs |
| chr12 | 2026839 | 2027354 | 1.52E−07 | 44.78231293 | Hyper-methylated | PCa\|NT DMRs |
| chr12 | 2030167 | 2030504 | 5.84E−05 | 49.95436508 | Hyper-methylated | PCa\|NT DMRs |
| chr12 | 2322888 | 2323091 | 0.001969641 | 38.07142857 | Hyper-methylated | PCa\|NT DMRs |
| chr12 | 2339290 | 2339614 | 5.25E−10 | 26.10770975 | Hyper-methylated | PCa\|NT DMRs |
| chr12 | 2612863 | 2613222 | 0.008529021 | 27.36904762 | Hyper-methylated | PCa\|NT DMRs |
| chr12 | 3053221 | 3054641 | 0.004505524 | −25.1265873 | Hypo-methylated | PCa\|NT DMRs |
| chr12 | 3070096 | 3073324 | 0.000688325 | 23.14725275 | Hyper-methylated | PCa\|NT DMRs |
| chr12 | 3947938 | 3948441 | 7.10E−13 | 42.14937343 | Hyper-methylated | PCa\|NT DMRs |
| chr12 | 4518923 | 4535585 | 0.000715461 | −28.33849206 | Hypo-methylated | PCa\|NT DMRs |
| chr12 | 4828343 | 4899022 | 0.000451335 | −25.08955453 | Hypo-methylated | PCa\|NT DMRs |
| chr12 | 4919618 | 4920724 | 3.37E−08 | −27.98796296 | Hypo-methylated | PCa\|NT DMRs |
| chr12 | 4945911 | 5003325 | 3.70E−07 | −29.63571429 | Hypo-methylated | PCa\|NT DMRs |
| chr12 | 5155093 | 5156934 | 7.44E−06 | −46.98241758 | Hypo-methylated | PCa\|NT DMRs |
| chr12 | 5404354 | 5414842 | 0.005968871 | −27.49142857 | Hypo-methylated | PCa\|NT DMRs |
| chr12 | 5542167 | 5542931 | 1.21E−11 | 21.76585366 | Hyper-methylated | PCa\|NT DMRs |
| chr12 | 5576109 | 5576272 | 0.001241245 | −29.03439153 | Hypo-methylated | PCa\|NT DMRs |
| chr12 | 5997135 | 5997258 | 0.000974795 | 37.76785714 | Hyper-methylated | PCa\|NT DMRs |
| chr12 | 6178534 | 6184558 | 1.13E−05 | 44.17687075 | Hyper-methylated | PCa\|NT DMRs |
| chr12 | 6199418 | 6214697 | 0.005968871 | 23.64814815 | Hyper-methylated | PCa\|NT DMRs |
| chr12 | 6319365 | 6337370 | 2.32E−05 | −29.81162465 | Hypo-methylated | PCa\|NT DMRs |
| chr12 | 6341734 | 6341821 | 0.00064571 | 35.7755102 | Hyper-methylated | PCa\|NT DMRs |
| chr12 | 6484016 | 6485475 | 0.001139334 | −36.74563492 | Hypo-methylated | PCa\|NT DMRs |
| chr12 | 6493402 | 6494457 | 0.008792234 | −30.35714286 | Hypo-methylated | PCa\|NT DMRs |
| chr12 | 6641241 | 6641856 | 0.00179283 | 30.6961039 | Hyper-methylated | PCa\|NT DMRs |
| chr12 | 6803209 | 6804725 | 0.001175696 | −26.48823529 | Hypo-methylated | PCa\|NT DMRs |
| chr12 | 6876688 | 6880022 | 0.000174611 | −23.27192118 | Hypo-methylated | PCa\|NT DMRs |
| chr12 | 6898647 | 6901984 | 0.000652677 | 37.70912698 | Hyper-methylated | PCa\|NT DMRs |
| chr12 | 6933745 | 6936153 | 8.74E−07 | −29.15619048 | Hypo-methylated | PCa\|NT DMRs |
| chr12 | 7044298 | 7046620 | 9.50E−05 | 20.95604396 | Hyper-methylated | PCa\|NT DMRs |
| chr12 | 7059950 | 7066499 | 7.82E−06 | 41.83139881 | Hyper-methylated | PCa\|NT DMRs |
| chr12 | 7066939 | 7069094 | 0.00240833 | 26.86589862 | Hyper-methylated | PCa\|NT DMRs |
| chr12 | 7071796 | 7079106 | 2.50E−08 | −35.94417077 | Hypo-methylated | PCa\|NT DMRs |
| chr12 | 7873132 | 7892159 | 0.000570576 | −25.74338624 | Hypo-methylated | PCa\|NT DMRs |
| chr12 | 8148528 | 8170758 | 0.00017871 | −29.46333333 | Hypo-methylated | PCa\|NT DMRs |
| chr12 | 8171468 | 8171769 | 4.11E−07 | 21.85714286 | Hyper-methylated | PCa\|NT DMRs |
| chr12 | 8320598 | 8322550 | 0.00064571 | −28.93877551 | Hypo-methylated | PCa\|NT DMRs |
| chr12 | 8413831 | 8490760 | 6.78E−07 | −26.77595238 | Hypo-methylated | PCa\|NT DMRs |
| chr12 | 8556792 | 8839279 | 1.11E−19 | −27.72018349 | Hypo-methylated | PCa\|NT DMRs |
| chr12 | 9603044 | 9603251 | 6.42E−05 | 21.82275132 | Hyper-methylated | PCa\|NT DMRs |
| chr12 | 9892632 | 9901742 | 0.00179283 | −39.88988095 | Hypo-methylated | PCa\|NT DMRs |
| chr12 | 9966938 | 10028922 | 0.002437742 | −34.07671958 | Hypo-methylated | PCa\|NT DMRs |
| chr12 | 10029037 | 10182598 | 1.09E−07 | −26.39189463 | Hypo-methylated | PCa\|NT DMRs |
| chr12 | 10185237 | 10253227 | 6.67E−10 | −31.73061224 | Hypo-methylated | PCa\|NT DMRs |
| chr12 | 10291998 | 10299495 | 0.000364072 | −21.69413919 | Hypo-methylated | PCa\|NT DMRs |
| chr12 | 10317786 | 10330979 | 0.00064571 | −30.89387755 | Hypo-methylated | PCa\|NT DMRs |
| chr12 | 11700234 | 11709909 | 4.80E−06 | −38.16051587 | Hypo-methylated | PCa\|NT DMRs |
| chr12 | 11809631 | 11944107 | 0.000635188 | 29.64696223 | Hyper-methylated | PCa\|NT DMRs |
| chr12 | 13080235 | 13080714 | 1.97E−05 | −35.70095238 | Hypo-methylated | PCa\|NT DMRs |
| chr12 | 13426961 | 13505997 | 0.001181579 | −21.85059524 | Hypo-methylated | PCa\|NT DMRs |
| chr12 | 14720581 | 14721015 | 3.00E−08 | 24.51751701 | Hyper-methylated | PCa\|NT DMRs |
| chr12 | 15484014 | 15626344 | 1.41E−11 | −33.94375804 | Hypo-methylated | PCa\|NT DMRs |
| chr12 | 16883705 | 17164660 | 5.68E−08 | −26.17206349 | Hypo-methylated | PCa\|NT DMRs |
| chr12 | 17497525 | 17569867 | 1.42E−05 | −33.63673469 | Hypo-methylated | PCa\|NT DMRs |
| chr12 | 17584274 | 18526644 | 2.85E−32 | −33.9105723 | Hypo-methylated | PCa\|NT DMRs |
| chr12 | 18857556 | 19062463 | 4.02E−05 | −23.40687831 | Hypo-methylated | PCa\|NT DMRs |
| chr12 | 21780466 | 21801327 | 0.000205174 | −26.83928571 | Hypo-methylated | PCa\|NT DMRs |
| chr12 | 21976755 | 22049398 | 1.97E−05 | −28.25142857 | Hypo-methylated | PCa\|NT DMRs |
| chr12 | 22486861 | 22487569 | 2.17E−07 | 37.73076923 | Hyper-methylated | PCa\|NT DMRs |
| chr12 | 23406213 | 23713145 | 2.32E−06 | −27.8789916 | Hypo-methylated | PCa\|NT DMRs |
| chr12 | 23883855 | 23977802 | 6.42E−05 | −23.51957672 | Hypo-methylated | PCa\|NT DMRs |
| chr12 | 25079205 | 25087001 | 5.33E−05 | −22.16190476 | Hypo-methylated | PCa\|NT DMRs |
| chr12 | 26427859 | 26430998 | 0.000145094 | 37.40992063 | Hyper-methylated | PCa\|NT DMRs |
| chr12 | 27862851 | 27863044 | 0.000847246 | −26.75457875 | Hypo-methylated | PCa\|NT DMRs |
| chr12 | 28123939 | 28128618 | 5.06E−15 | 26.14457455 | Hyper-methylated | PCa\|NT DMRs |
| chr12 | 30672087 | 30672306 | 0.001969641 | −20.63492063 | Hypo-methylated | PCa\|NT DMRs |
| chr12 | 32163366 | 32163399 | 0.001969641 | −53.08333333 | Hypo-methylated | PCa\|NT DMRs |
| chr12 | 32330133 | 32334246 | 0.001111028 | −23.2989418 | Hypo-methylated | PCa\|NT DMRs |
| chr12 | 34477568 | 34483952 | 3.53E−08 | −28.12594417 | Hypo-methylated | PCa\|NT DMRs |
| chr12 | 34488423 | 34488683 | 8.32E−06 | −25.76642857 | Hypo-methylated | PCa\|NT DMRs |
| chr12 | 34488817 | 34489037 | 0.000746346 | −23.45904762 | Hypo-methylated | PCa\|NT DMRs |
| chr12 | 34489145 | 34489397 | 0.000325529 | −24.82857143 | Hypo-methylated | PCa\|NT DMRs |
| chr12 | 34489518 | 34489774 | 1.48E−05 | −28.27329193 | Hypo-methylated | PCa\|NT DMRs |
| chr12 | 34489878 | 34490201 | 2.61E−07 | −36.86539683 | Hypo-methylated | PCa\|NT DMRs |
| chr12 | 34490425 | 34491459 | 1.56E−13 | −35.07952381 | Hypo-methylated | PCa\|NT DMRs |
| chr12 | 34492707 | 34492747 | 0.00641359 | −23.70238095 | Hypo-methylated | PCa\|NT DMRs |
| chr12 | 34492830 | 34492939 | 0.00179283 | −30.29264069 | Hypo-methylated | PCa\|NT DMRs |
| chr12 | 34494176 | 34494955 | 3.24E−10 | −33.2031746 | Hypo-methylated | PCa\|NT DMRs |
| chr12 | 34495257 | 34495730 | 0.000200681 | −22.77454844 | Hypo-methylated | PCa\|NT DMRs |
| chr12 | 34497838 | 34498033 | 0.002097193 | −33.34693878 | Hypo-methylated | PCa\|NT DMRs |
| chr12 | 34498501 | 34499074 | 3.45E−07 | −20.97009967 | Hypo-methylated | PCa\|NT DMRs |
| chrl2 | 34499417 | 34500449 | 2.02E−13 | −33.60384615 | Hypo-methylated | PCa\|NT DMRs |
| chr12 | 34500923 | 34501516 | 2.82E−07 | −37.93849206 | Hypo-methylated | PCa\|NT DMRs |
| chr12 | 34502636 | 34503112 | 7.57E−05 | −36.90952381 | Hypo-methylated | PCa\|NT DMRs |
| chr12 | 38551678 | 38553739 | 2.97E−20 | −24.76375 | Hypo-methylated | PCa\|NT DMRs |
| chr12 | 40628426 | 41040444 | 7.55E−12 | −32.6170068 | Hypo-methylated | PCa\|NT DMRs |
| chr12 | 41087584 | 41160083 | 0.005968871 | −28.95904762 | Hypo-methylated | PCa\|NT DMRs |
| chr12 | 41212912 | 41272044 | 0.00064571 | −35.93197279 | Hypo-methylated | PCa\|NT DMRs |
| chr12 | 41966930 | 42275153 | 1.18E−08 | −28.08769841 | Hypo-methylated | PCa\|NT DMRs |
| chr12 | 43781764 | 43908340 | 1.18E−08 | −37.65 | Hypo-methylated | PCa\|NT DMRs |
| chr12 | 43988798 | 44128510 | 1.25E−06 | −27.42057143 | Hypo-methylated | PCa\|NT DMRs |
| chr12 | 45443339 | 45444530 | 8.44E−09 | 21.16634921 | Hyper-methylated | PCa\|NT DMRs |
| chr12 | 46663445 | 46663695 | 4.62E−05 | 43.91555556 | Hyper-methylated | PCa\|NT DMRs |
| chr12 | 46767122 | 46767329 | 3.44E−06 | 36.70654762 | Hyper-methylated | PCa\|NT DMRs |
| chr12 | 47224672 | 47225065 | 1.72E−05 | 27.76870748 | Hyper-methylated | PCa\|NT DMRs |
| chr12 | 47937757 | 47952036 | 0.000377317 | −25.19795918 | Hypo-methylated | PCa\|NT DMRs |
| chr12 | 48275613 | 48277153 | 0.005968871 | 34.23809524 | Hyper-methylated | PCa\|NT DMRs |
| chr12 | 48599695 | 48665827 | 0.00064571 | −23.28571429 | Hypo-methylated | PCa\|NT DMRs |
| chr12 | 49297470 | 49298352 | 0.004543362 | 28.43223443 | Hyper-methylated | PCa\|NT DMRs |
| chr12 | 49363774 | 49364578 | 1.55E−06 | 21.02364532 | Hyper-methylated | PCa\|NT DMRs |
| chr12 | 49626915 | 49627711 | 0.000118733 | 39.11904762 | Hyper-methylated | PCa\|NT DMRs |
| chr12 | 49688942 | 49689474 | 1.57E−07 | 30.52248677 | Hyper-methylated | PCa\|NT DMRs |
| chr12 | 49726478 | 49730313 | 1.07E−08 | 31.48921283 | Hyper-methylated | PCa\|NT DMRs |
| chr12 | 49731598 | 49736288 | 1.67E−07 | 21.88538588 | Hyper-methylated | PCa\|NT DMRs |
| chr12 | 49780244 | 49783223 | 0.005513661 | 32.13571429 | Hyper-methylated | PCa\|NT DMRs |
| chr12 | 49943396 | 49943435 | 0.005968871 | −29.48571429 | Hypo-methylated | PCa\|NT DMRs |
| chr12 | 50099883 | 50100668 | 0.000187876 | 35.15079365 | Hyper-methylated | PCa\|NT DMRs |
| chr12 | 50297363 | 50297988 | 2.63E−09 | 29.6899696 | Hyper-methylated | PCa\|NT DMRs |
| chr12 | 50367487 | 50376049 | 0.00046742 | −48.94545455 | Hypo-methylated | PCa\|NT DMRs |
| chr12 | 50432893 | 50435658 | 0.005513661 | −24.70068027 | Hypo-methylated | PCa\|NT DMRs |
| chr12 | 50497810 | 50498024 | 0.000481535 | −44.74603175 | Hypo-methylated | PCa\|NT DMRs |
| chr12 | 50506785 | 50507180 | 0.005513661 | −40.0922619 | Hypo-methylated | PCa\|NT DMRs |
| chr12 | 50912168 | 50914115 | 0.003391914 | 34.55555556 | Hyper-methylated | PCa\|NT DMRs |
| chr12 | 51712324 | 51718251 | 7.44E−10 | 56.93809524 | Hyper-methylated | PCa\|NT DMRs |
| chr12 | 51729468 | 51739881 | 0.001115302 | 20.70654762 | Hyper-methylated | PCa\|NT DMRs |
| chr12 | 51785969 | 51788039 | 2.64E−08 | 29.53261183 | Hyper-methylated | PCa\|NT DMRs |
| chr12 | 52317218 | 52317733 | 0.002034353 | 21.16326531 | Hyper-methylated | PCa\|NT DMRs |
| chr12 | 52400495 | 52401828 | 3.27E−32 | 29.62617013 | Hyper-methylated | PCa\|NT DMRs |
| chr12 | 52509677 | 52511985 | 0.00064571 | −44.88503401 | Hypo-methylated | PCa\|NT DMRs |
| chr12 | 52715006 | 52715044 | 0.001969641 | −24.33333333 | Hypo-methylated | PCa\|NT DMRs |
| chr12 | 53128380 | 53130503 | 0.005968871 | −32.09714286 | Hypo-methylated | PCa\|NT DMRs |
| chr12 | 53207442 | 53212769 | 0.001426157 | −23.10084034 | Hypo-methylated | PCa\|NT DMRs |
| chr12 | 53268065 | 53268290 | 3.05E−09 | 25.05494505 | Hyper-methylated | PCa\|NT DMRs |
| chr12 | 53298237 | 53298969 | 0.001111028 | −39.9047619 | Hypo-methylated | PCa\|NT DMRs |
| chr12 | 53359203 | 53359419 | 0.000480383 | 31.14285714 | Hyper-methylated | PCa\|NT DMRs |
| chr12 | 53441697 | 53446877 | 0.000516057 | −33.92307692 | Hypo-methylated | PCa\|NT DMRs |
| chr12 | 53617239 | 53617383 | 2.86E−05 | 29.03296703 | Hyper-methylated | PCa\|NT DMRs |
| chr12 | 53836400 | 53836517 | 0.005968871 | 34.38761905 | Hyper-methylated | PCa\|NT DMRs |
| chr12 | 54131102 | 54135269 | 0.004227093 | 25.69708995 | Hyper-methylated | PCa\|NT DMRs |
| chr12 | 54321497 | 54330957 | 3.33E−10 | 21.83178054 | Hyper-methylated | PCa\|NT DMRs |
| chr12 | 54376047 | 54380101 | 4.09E−15 | 27.38863287 | Hyper-methylated | PCa\|NT DMRs |
| chr12 | 54407959 | 54409526 | 9.69E−12 | 40.35896359 | Hyper-methylated | PCa\|NT DMRs |
| chr12 | 54428362 | 54454375 | 2.16E−33 | 39.01127232 | Hyper-methylated | PCa\|NT DMRs |
| chr12 | 54474265 | 54475553 | 0.005968871 | 33.82857143 | Hyper-methylated | PCa\|NT DMRs |
| chr12 | 54500141 | 54500787 | 3.60E−10 | 32.46699507 | Hyper-methylated | PCa\|NT DMRs |
| chr12 | 54719728 | 54719764 | 0.001969641 | −39.5952381 | Hypo-methylated | PCa\|NT DMRs |
| chr12 | 54755304 | 54756713 | 0.004236974 | −20.72571429 | Hypo-methylated | PCa\|NT DMRs |
| chr12 | 54806218 | 54812532 | 0.000238731 | 33.46078431 | Hyper-methylated | PCa\|NT DMRs |
| chr12 | 56115070 | 56120638 | 0.004071561 | −25.31587302 | Hypo-methylated | PCa\|NT DMRs |
| chr12 | 57094398 | 57094646 | 0.000481535 | 53.51851852 | Hyper-methylated | PCa\|NT DMRs |
| chr12 | 57545775 | 57546086 | 0.001969641 | −28.8468254 | Hypo-methylated | PCa\|NT DMRs |
| chr12 | 57550599 | 57550897 | 4.28E−07 | 41.99428571 | Hyper-methylated | PCa\|NT DMRs |
| chr12 | 57634856 | 57635744 | 4.42E−05 | 25.47985348 | Hyper-methylated | PCa\|NT DMRs |
| chr12 | 57958739 | 57963109 | 0.005968871 | −25.18666667 | Hypo-methylated | PCa\|NT DMRs |
| chr12 | 58119674 | 58122083 | 7.14E−09 | −46.36507937 | Hypo-methylated | PCa\|NT DMRs |
| chr12 | 59395863 | 59981266 | 4.85E−16 | −31.45753158 | Hypo-methylated | PCa\|NT DMRs |
| chr12 | 60008487 | 60225335 | 4.11E−07 | −32.64987469 | Hypo-methylated | PCa\|NT DMRs |
| chr12 | 60619477 | 60642817 | 0.00064571 | −33.83401361 | Hypo-methylated | PCa\|NT DMRs |
| chr12 | 60782219 | 61788649 | 2.04E−17 | −21.89330573 | Hypo-methylated | PCa\|NT DMRs |
| chr12 | 62266135 | 62510885 | 1.91E−07 | −21.53358396 | Hypo-methylated | PCa\|NT DMRs |
| chr12 | 65218335 | 65220299 | 2.27E−27 | 38.01987818 | Hyper-methylated | PCa\|NT DMRs |
| chr12 | 65515438 | 65515703 | 3.77E−05 | 34.02946429 | Hyper-methylated | PCa\|NT DMRs |
| chr12 | 65672082 | 65672234 | 0.000836132 | 24.46190476 | Hyper-methylated | PCa\|NT DMRs |
| chr12 | 65801968 | 65997461 | 0.000133155 | −31.87103175 | Hypo-methylated | PCa\|NT DMRs |
| chr12 | 66275570 | 66276260 | 1.02E−09 | 32.65873016 | Hyper-methylated | PCa\|NT DMRs |
| chr12 | 68757487 | 68759201 | 0.000949805 | 33.87103175 | Hyper-methylated | PCa\|NT DMRs |
| chr12 | 68825940 | 68848995 | 0.00641359 | −41.41587302 | Hypo-methylated | PCa\|NT DMRs |
| chr12 | 72079236 | 72079323 | 0.00064571 | −48.46938776 | Hypo-methylated | PCa\|NT DMRs |
| chr12 | 72395098 | 72645075 | 1.32E−08 | −28.28265306 | Hypo-methylated | PCa\|NT DMRs |
| chr12 | 74635771 | 74665046 | 8.05E−06 | −21.05619048 | Hypo-methylated | PCa\|NT DMRs |
| chr12 | 75960802 | 76117780 | 6.42E−05 | −23.58095238 | Hypo-methylated | PCa\|NT DMRs |
| chr12 | 82246247 | 82286973 | 0.001969641 | −32 | Hypo-methylated | PCa\|NT DMRs |
| chr12 | 84274211 | 84433624 | 0.001241245 | −29.14867725 | Hypo-methylated | PCa\|NT DMRs |
| chr12 | 84518549 | 84549192 | 3.05E−06 | −30.50769231 | Hypo-methylated | PCa\|NT DMRs |
| chr12 | 85187989 | 85229497 | 1.42E−05 | −40.23979592 | Hypo-methylated | PCa\|NT DMRs |
| chr12 | 85896678 | 86215567 | 1.26E−05 | −31.45291005 | Hypo-methylated | PCa\|NT DMRs |
| chr12 | 86407124 | 86434887 | 1.03E−05 | −30.42857143 | Hypo-methylated | PCa\|NT DMRs |
| chr12 | 87781481 | 88226255 | 1.41E−07 | −24.08712522 | Hypo-methylated | PCa\|NT DMRs |
| chr12 | 89744877 | 89745193 | 0.00016423 | 23.41501832 | Hyper-methylated | PCa\|NT DMRs |
| chr12 | 94543850 | 94568501 | 4.42E−08 | 20.6626294 | Hyper-methylated | PCa\|NT DMRs |
| chr12 | 94626160 | 94658281 | 0.006025732 | −34.86428571 | Hypo-methylated | PCa\|NT DMRs |
| chr12 | 95038193 | 95039405 | 0.005513661 | 42.05357143 | Hyper-methylated | PCa\|NT DMRs |
| chr12 | 95840421 | 95840829 | 0.000165902 | 40.11941392 | Hyper-methylated | PCa\|NT DMRs |
| chr12 | 96183288 | 96183883 | 1.05E−10 | 27.01123321 | Hyper-methylated | PCa\|NT DMRs |
| chr12 | 96388463 | 96395696 | 0.000123553 | 20.89635854 | Hyper-methylated | PCa\|NT DMRs |
| chr12 | 98767469 | 98781960 | 0.00641359 | −40 | Hypo-methylated | PCa\|NT DMRs |
| chr12 | 100774654 | 100779268 | 0.002585277 | −27.48809524 | Hypo-methylated | PCa\|NT DMRs |
| chr12 | 103498427 | 103498511 | 0.005968871 | −38.03333333 | Hypo-methylated | PCa\|NT DMRs |
| chr12 | 103695950 | 103696238 | 1.52E−07 | 27.57891156 | Hyper-methylated | PCa\|NT DMRs |
| chr12 | 104697450 | 104697846 | 1.61E−10 | 29.16885457 | Hyper-methylated | PCa\|NT DMRs |
| chr12 | 104851871 | 104853159 | 9.54E−13 | 32.41496599 | Hyper-methylated | PCa\|NT DMRs |
| chr12 | 105107480 | 105109310 | 0.005968871 | −33.4 | Hypo-methylated | PCa\|NT DMRs |
| chr12 | 105477686 | 105477733 | 0.00641359 | 23.33333333 | Hyper-methylated | PCa\|NT DMRs |
| chr12 | 106022076 | 106022112 | 0.005968871 | −30.84 | Hypo-methylated | PCa\|NT DMRs |
| chr12 | 106531772 | 106532142 | 0.000111427 | 29.58285714 | Hyper-methylated | PCa\|NT DMRs |
| chr12 | 106533464 | 106533737 | 8.47E−06 | 29.96969697 | Hyper-methylated | PCa\|NT DMRs |
| chr12 | 107039031 | 107039098 | 0.001969641 | −51.04761905 | Hypo-methylated | PCa\|NT DMRs |
| chr12 | 107297126 | 107297326 | 0.002437742 | 24.28835979 | Hyper-methylated | PCa\|NT DMRs |
| chr12 | 108347105 | 108399657 | 0.001969641 | −37.0515873 | Hypo-methylated | PCa\|NT DMRs |
| chr12 | 109162275 | 109162678 | 2.99E−07 | 28.71530612 | Hyper-methylated | PCa\|NT DMRs |
| chr12 | 109273780 | 109278217 | 0.003871035 | −27.56785714 | Hypo-methylated | PCa\|NT DMRs |
| chr12 | 109297307 | 109299060 | 0.001241245 | 24.76190476 | Hyper-methylated | PCa\|NT DMRs |
| chr12 | 109526810 | 109527869 | 0.006589535 | −30.18857143 | Hypo-methylated | PCa\|NT DMRs |
| chr12 | 109711168 | 109711332 | 0.001969641 | 33.45238095 | Hyper-methylated | PCa\|NT DMRs |
| chr12 | 109716837 | 109716969 | 0.00064571 | −45.07142857 | Hypo-methylated | PCa\|NT DMRs |
| chr12 | 109729299 | 109729961 | 3.87E−06 | 30.06893424 | Hyper-methylated | PCa\|NT DMRs |
| chr12 | 110156175 | 110157510 | 1.66E−05 | 36.91456583 | Hyper-methylated | PCa\|NT DMRs |
| chr12 | 110265801 | 110266248 | 6.69E−05 | 50.88190476 | Hyper-methylated | PCa\|NT DMRs |
| chr12 | 111112991 | 111117302 | 0.005968871 | −32.98 | Hypo-methylated | PCa\|NT DMRs |
| chr12 | 111331137 | 111331381 | 0.001969641 | −40.22619048 | Hypo-methylated | PCa\|NT DMRs |
| chr12 | 111456714 | 111456855 | 0.004095673 | −34.50793651 | Hypo-methylated | PCa\|NT DMRs |
| chr12 | 111470768 | 111470828 | 0.001969641 | 41.39047619 | Hyper-methylated | PCa\|NT DMRs |
| chr12 | 111593459 | 111596411 | 0.007305013 | 22.31547619 | Hyper-methylated | PCa\|NT DMRs |
| chr12 | 111599699 | 111604656 | 0.007305013 | −46.46785714 | Hypo-methylated | PCa\|NT DMRs |
| chr12 | 111798936 | 111799226 | 0.00064571 | 37.85714286 | Hyper-methylated | PCa\|NT DMRs |
| chr12 | 111842687 | 111842940 | 6.42E−05 | 31.69417989 | Hyper-methylated | PCa\|NT DMRs |
| chr12 | 111844152 | 111847845 | 3.10E−12 | 36.89194139 | Hyper-methylated | PCa\|NT DMRs |
| chr12 | 112591144 | 112591636 | 0.005968871 | −27.4 | Hypo-methylated | PCa\|NT DMRs |
| chr12 | 112835090 | 112835526 | 0.008482405 | −34.77142857 | Hypo-methylated | PCa\|NT DMRs |
| chr12 | 113031027 | 113229124 | 4.40E−07 | −24.31471861 | Hypo-methylated | PCa\|NT DMRs |
| chr12 | 113234577 | 113307766 | 1.15E−08 | −29.43756614 | Hypo-methylated | PCa\|NT DMRs |
| chr12 | 113499672 | 113504056 | 0.000301781 | 23.0517316 | Hyper-methylated | PCa\|NT DMRs |
| chr12 | 113521291 | 113531394 | 0.000346222 | 20.35289575 | Hyper-methylated | PCa\|NT DMRs |
| chr12 | 113679215 | 113684696 | 0.001216064 | −26.65483871 | Hypo-methylated | PCa\|NT DMRs |
| chr12 | 113770527 | 113771942 | 0.006207271 | 30.31190476 | Hyper-methylated | PCa\|NT DMRs |
| chr12 | 114162770 | 114163053 | 1.97E−05 | −53.27809524 | Hypo-methylated | PCa\|NT DMRs |
| chr12 | 114192081 | 114204098 | 2.45E−07 | −31.16929392 | Hypo-methylated | PCa\|NT DMRs |
| chr12 | 115133106 | 115152344 | 4.14E−05 | 46.20972222 | Hyper-methylated | PCa\|NT DMRs |
| chr12 | 115153881 | 115174322 | 6.44E−06 | 35.96550265 | Hyper-methylated | PCa\|NT DMRs |
| chr12 | 115573770 | 115576790 | 0.007560755 | −34.97777778 | Hypo-methylated | PCa\|NT DMRs |
| chr12 | 116037353 | 116062028 | 3.96E−05 | 32.35892857 | Hyper-methylated | PCa\|NT DMRs |
| chr12 | 116278193 | 116278292 | 0.005968871 | −46.06857143 | Hypo-methylated | PCa\|NT DMRs |
| chr12 | 116716409 | 116724332 | 6.42E−05 | 41.56402116 | Hyper-methylated | PCa\|NT DMRs |
| chr12 | 116831310 | 116831525 | 0.005513661 | 30.3877551 | Hyper-methylated | PCa\|NT DMRs |
| chr12 | 116922248 | 116922446 | 0.001181579 | −26.67346939 | Hypo-methylated | PCa\|NT DMRs |
| chr12 | 116946159 | 116946306 | 7.70E−09 | 29.45308642 | Hyper-methylated | PCa\|NT DMRs |
| chr12 | 116996796 | 116997121 | 0.000176046 | 50.85281385 | Hyper-methylated | PCa\|NT DMRs |
| chr12 | 117096354 | 117096479 | 0.003622801 | −30.96666667 | Hypo-methylated | PCa\|NT DMRs |
| chr12 | 117129243 | 117133383 | 2.29E−06 | 28.6505291 | Hyper-methylated | PCa\|NT DMRs |
| chr12 | 117135779 | 117155674 | 0.000188172 | −28.63756614 | Hypo-methylated | PCa\|NT DMRs |
| chr12 | 117477883 | 117478603 | 0.000117699 | 43.87012987 | Hyper-methylated | PCa\|NT DMRs |
| chr12 | 117482953 | 117486917 | 0.000363991 | 31.76296296 | Hyper-methylated | PCa\|NT DMRs |
| chr12 | 117501297 | 117501486 | 0.002437742 | 46.03809524 | Hyper-methylated | PCa\|NT DMRs |
| chr12 | 117580082 | 117583972 | 0.000141004 | −34.20502646 | Hypo-methylated | PCa\|NT DMRs |
| chr12 | 117658428 | 117658788 | 0.001969641 | −31.45634921 | Hypo-methylated | PCa\|NT DMRs |
| chr12 | 117771728 | 117772769 | 0.00641359 | −31.93650794 | Hypo-methylated | PCa\|NT DMRs |
| chr12 | 118569390 | 118569736 | 0.00064571 | −38.18367347 | Hypo-methylated | PCa\|NT DMRs |
| chr12 | 119070924 | 119206291 | 0.006225823 | −22.72510823 | Hypo-methylated | PCa\|NT DMRs |
| chr12 | 119216863 | 119407630 | 0.000567209 | −27.97619048 | Hypo-methylated | PCa\|NT DMRs |
| chr12 | 119653771 | 119695299 | 0.00051204 | −25.98761905 | Hypo-methylated | PCa\|NT DMRs |
| chr12 | 120106954 | 120107676 | 0.000167524 | −24.59275362 | Hypo-methylated | PCa\|NT DMRs |
| chr12 | 120700871 | 120701175 | 0.000570576 | −37.57354497 | Hypo-methylated | PCa\|NT DMRs |
| chr12 | 120799332 | 120799725 | 2.39E−08 | 42.09213251 | Hyper-methylated | PCa\|NT DMRs |
| chr12 | 120877112 | 120877187 | 0.001969641 | −35.49603175 | Hypo-methylated | PCa\|NT DMRs |
| chr12 | 120973751 | 120975323 | 0.003122338 | −32.49603175 | Hypo-methylated | PCa\|NT DMRs |
| chr12 | 121456139 | 121456255 | 0.00179283 | 28.02380952 | Hyper-methylated | PCa\|NT DMRs |
| chr12 | 121565718 | 121565847 | 0.005968871 | −24.55428571 | Hypo-methylated | PCa\|NT DMRs |
| chr12 | 121716976 | 121728784 | 3.96E−08 | −37.09365079 | Hypo-methylated | PCa\|NT DMRs |
| chr12 | 121735816 | 121735963 | 0.00064571 | −42.3877551 | Hypo-methylated | PCa\|NT DMRs |
| chr12 | 121881969 | 121882219 | 1.76E−06 | −37.26190476 | Hypo-methylated | PCa\|NT DMRs |
| chr12 | 121890460 | 121890799 | 0.001391421 | 24.93896104 | Hyper-methylated | PCa\|NT DMRs |
| chr12 | 122014850 | 122017085 | 4.42E−12 | 30.87284226 | Hyper-methylated | PCa\|NT DMRs |
| chr12 | 122018897 | 122020662 | 0.00603903 | 29.97671958 | Hyper-methylated | PCa\|NT DMRs |
| chr12 | 122233453 | 122237290 | 2.86E−06 | −39.08822055 | Hypo-methylated | PCa\|NT DMRs |
| chr12 | 122243926 | 122248654 | 8.34E−08 | −23.51671335 | Hypo-methylated | PCa\|NT DMRs |
| chr12 | 122356025 | 122356144 | 6.42E−05 | −21.89153439 | Hypo-methylated | PCa\|NT DMRs |
| chr12 | 122444219 | 122444664 | 0.000149331 | 20.66233766 | Hyper-methylated | PCa\|NT DMRs |
| chr12 | 122458822 | 122458917 | 0.000250214 | −25.61904762 | Hypo-methylated | PCa\|NT DMRs |
| chr12 | 122622501 | 122622628 | 0.000481535 | −30.98412698 | Hypo-methylated | PCa\|NT DMRs |
| chr12 | 122669143 | 122669499 | 6.69E−05 | 51.23571429 | Hyper-methylated | PCa\|NT DMRs |
| chr12 | 123131635 | 123131816 | 0.001969641 | 62.47619048 | Hyper-methylated | PCa\|NT DMRs |
| chr12 | 123350609 | 123350956 | 0.007679542 | 26.36507937 | Hyper-methylated | PCa\|NT DMRs |
| chr12 | 123428003 | 123432025 | 0.001170059 | 36.55380952 | Hyper-methylated | PCa\|NT DMRs |
| chr12 | 123454719 | 123458517 | 0.00064571 | −43.71428571 | Hypo-methylated | PCa\|NT DMRs |
| chr12 | 123469208 | 123469588 | 3.36E−06 | 32.4622449 | Hyper-methylated | PCa\|NT DMRs |
| chr12 | 123516088 | 123519310 | 0.000475166 | 39.64761905 | Hyper-methylated | PCa\|NT DMRs |
| chr12 | 123738801 | 123745121 | 0.000205174 | −39.28511905 | Hypo-methylated | PCa\|NT DMRs |
| chr12 | 123877672 | 123884045 | 4.90E−06 | −46.30505952 | Hypo-methylated | PCa\|NT DMRs |
| chr12 | 124539311 | 124539401 | 0.005968871 | 21.00571429 | Hyper-methylated | PCa\|NT DMRs |
| chr12 | 124770923 | 124774533 | 0.003764982 | −28.99801587 | Hypo-methylated | PCa\|NT DMRs |
| chr12 | 124779455 | 124779574 | 0.008581782 | 20.77777778 | Hyper-methylated | PCa\|NT DMRs |
| chr12 | 124780181 | 124781210 | 5.12E−11 | 38.87738095 | Hyper-methylated | PCa\|NT DMRs |
| chr12 | 124781699 | 124781926 | 0.00064571 | −44.7755102 | Hypo-methylated | PCa\|NT DMRs |
| chr12 | 124841219 | 124846100 | 4.03E−05 | −29.51661601 | Hypo-methylated | PCa\|NT DMRs |
| chr12 | 124853894 | 124856388 | 2.57E−11 | −58.2122449 | Hypo-methylated | PCa\|NT DMRs |
| chr12 | 124864594 | 124864658 | 0.005968871 | 34.57142857 | Hyper-methylated | PCa\|NT DMRs |
| chr12 | 124877457 | 124889142 | 3.38E−05 | −23.55468254 | Hypo-methylated | PCa\|NT DMRs |
| chr12 | 124905648 | 124908130 | 0.000229601 | −39.37946429 | Hypo-methylated | PCa\|NT DMRs |
| chr12 | 124925728 | 124927552 | 0.001969641 | −24.17063492 | Hypo-methylated | PCa\|NT DMRs |
| chr12 | 124937758 | 124942464 | 2.06E−10 | 44.25765766 | Hyper-methylated | PCa\|NT DMRs |
| chr12 | 124950553 | 124951223 | 5.51E−08 | 51.71861472 | Hyper-methylated | PCa\|NT DMRs |
| chr12 | 124982371 | 124983008 | 0.000215808 | 35.66666667 | Hyper-methylated | PCa\|NT DMRs |
| chr12 | 124985381 | 124986238 | 0.00064571 | 29.15306122 | Hyper-methylated | PCa\|NT DMRs |
| chr12 | 124986459 | 124986722 | 0.000250214 | −35.29285714 | Hypo-methylated | PCa\|NT DMRs |
| chr12 | 125000485 | 125003430 | 1.41E−13 | −21.25889436 | Hypo-methylated | PCa\|NT DMRs |
| chr12 | 125028943 | 125030659 | 0.000349416 | 24.90225564 | Hyper-methylated | PCa\|NT DMRs |
| chr12 | 125076772 | 125091185 | 0.000924001 | 23.57192982 | Hyper-methylated | PCa\|NT DMRs |
| chr12 | 125101264 | 125101443 | 0.001969641 | 38.71428571 | Hyper-methylated | PCa\|NT DMRs |
| chr12 | 125258704 | 125258850 | 0.000205174 | 39.92678571 | Hyper-methylated | PCa\|NT DMRs |
| chr12 | 125322777 | 125322961 | 0.00064571 | −56.99319728 | Hypo-methylated | PCa\|NT DMRs |
| chr12 | 125341263 | 125341507 | 0.002534928 | 32.61857143 | Hyper-methylated | PCa\|NT DMRs |
| chr12 | 125353643 | 125353743 | 0.005968871 | 49.63714286 | Hyper-methylated | PCa\|NT DMRs |
| chr12 | 125413033 | 125413165 | 0.000205174 | 21.5 | Hyper-methylated | PCa\|NT DMRs |
| chr12 | 125670133 | 125670341 | 0.000635257 | 23.37254902 | Hyper-methylated | PCa\|NT DMRs |
| chr12 | 127545339 | 127630315 | 0.000207233 | −30.74732143 | Hypo-methylated | PCa\|NT DMRs |
| chr12 | 127631151 | 127645653 | 7.57E−05 | −28.73571429 | Hypo-methylated | PCa\|NT DMRs |
| chr12 | 127666101 | 127751370 | 0.000974795 | −37.81547619 | Hypo-methylated | PCa\|NT DMRs |
| chr12 | 127771410 | 127839087 | 5.94E−06 | −36.995671 | Hypo-methylated | PCa\|NT DMRs |
| chr12 | 127868235 | 127938771 | 4.10E−08 | −33.52666667 | Hypo-methylated | PCa\|NT DMRs |
| chr12 | 127941049 | 127962865 | 3.20E−05 | −33.25442177 | Hypo-methylated | PCa\|NT DMRs |
| chr12 | 127967686 | 128154845 | 1.17E−15 | −33.24277778 | Hypo-methylated | PCa\|NT DMRs |
| chr12 | 128210131 | 128235755 | 0.000179253 | −31.6007326 | Hypo-methylated | PCa\|NT DMRs |
| chr12 | 128335466 | 128383456 | 1.57E−07 | −35.27365079 | Hypo-methylated | PCa\|NT DMRs |
| chr12 | 128675569 | 128750043 | 0.003520801 | −30.51071429 | Hypo-methylated | PCa\|NT DMRs |
| chr12 | 128762836 | 128813879 | 1.69E−06 | −33.41344538 | Hypo-methylated | PCa\|NT DMRs |
| chr12 | 128852810 | 128865999 | 1.93E−06 | −32.86616541 | Hypo-methylated | PCa\|NT DMRs |
| chr12 | 129458299 | 129552362 | 2.35E−12 | −29.82289377 | Hypo-methylated | PCa\|NT DMRs |
| chr12 | 129702538 | 129716266 | 0.000377317 | −27.63809524 | Hypo-methylated | PCa\|NT DMRs |
| chr12 | 130300244 | 130317296 | 7.64E−07 | −23.36825397 | Hypo-methylated | PCa\|NT DMRs |
| chr12 | 130540502 | 130568557 | 2.54E−07 | −24.95044643 | Hypo-methylated | PCa\|NT DMRs |
| chr12 | 130580399 | 130585812 | 3.20E−05 | −31.65966387 | Hypo-methylated | PCa\|NT DMRs |
| chr12 | 130590155 | 130603569 | 9.42E−12 | −31.64875776 | Hypo-methylated | PCa\|NT DMRs |
| chr12 | 130612346 | 130640384 | 2.30E−17 | −31.96168582 | Hypo-methylated | PCa\|NT DMRs |
| chr12 | 130645328 | 130645375 | 0.000117507 | 24.61038961 | Hyper-methylated | PCa\|NT DMRs |
| chr12 | 130663490 | 130670959 | 2.95E−09 | −30.62236652 | Hypo-methylated | PCa\|NT DMRs |
| chr12 | 130680211 | 130683050 | 0.000106046 | −36.92857143 | Hypo-methylated | PCa\|NT DMRs |
| chr12 | 130699859 | 130753374 | 7.85E−11 | −31.72954823 | Hypo-methylated | PCa\|NT DMRs |
| chr12 | 130910143 | 130942579 | 9.00E−32 | −27.48298137 | Hypo-methylated | PCa\|NT DMRs |
| chr12 | 131096742 | 131122403 | 3.09E−05 | −25.62199546 | Hypo-methylated | PCa\|NT DMRs |
| chr12 | 131214254 | 131237782 | 6.47E−07 | −26.91795918 | Hypo-methylated | PCa\|NT DMRs |
| chr12 | 131245704 | 131260185 | 0.000363991 | −24.47759104 | Hypo-methylated | PCa\|NT DMRs |
| chr12 | 131260380 | 131260684 | 0.000652115 | 22.76923077 | Hyper-methylated | PCa\|NT DMRs |
| chr12 | 131439121 | 131462102 | 9.19E−10 | −24.33410138 | Hypo-methylated | PCa\|NT DMRs |
| chr12 | 131474131 | 131478792 | 1.13E−05 | −24.78864469 | Hypo-methylated | PCa\|NT DMRs |
| chr12 | 131492861 | 131496958 | 1.68E−07 | −29.31071429 | Hypo-methylated | PCa\|NT DMRs |
| chr12 | 131519674 | 131520766 | 0.000729802 | −35.7984127 | Hypo-methylated | PCa\|NT DMRs |
| chr12 | 131522919 | 131527770 | 2.52E−10 | −24.7997426 | Hypo-methylated | PCa\|NT DMRs |
| chr12 | 131588340 | 131589170 | 0.000570576 | −28.49450549 | Hypo-methylated | PCa\|NT DMRs |
| chr12 | 131604065 | 131607137 | 1.63E−08 | −26.21612903 | Hypo-methylated | PCa\|NT DMRs |
| chr12 | 131653302 | 131694221 | 8.73E−16 | −31.75753968 | Hypo-methylated | PCa\|NT DMRs |
| chr12 | 131812802 | 131863876 | 6.89E−19 | −29.5207483 | Hypo-methylated | PCa\|NT DMRs |
| chr12 | 131872360 | 131876009 | 0.001860863 | −24.2327381 | Hypo-methylated | PCa\|NT DMRs |
| chr12 | 131892700 | 131916572 | 5.88E−11 | −39.03392857 | Hypo-methylated | PCa\|NT DMRs |
| chr12 | 131923698 | 131933022 | 1.12E−08 | −28.00677656 | Hypo-methylated | PCa\|NT DMRs |
| chr12 | 131941562 | 132017866 | 1.82E−21 | −31.67383966 | Hypo-methylated | PCa\|NT DMRs |
| chr12 | 132041856 | 132106912 | 4.44E−37 | −29.70204518 | Hypo-methylated | PCa\|NT DMRs |
| chr12 | 132107115 | 132122144 | 4.31E−08 | −25.94790765 | Hypo-methylated | PCa\|NT DMRs |
| chr12 | 132336728 | 132337729 | 4.57E−06 | 34.46043956 | Hyper-methylated | PCa\|NT DMRs |
| chr12 | 132340000 | 132340675 | 0.000916248 | −24.93253968 | Hypo-methylated | PCa\|NT DMRs |
| chr12 | 132345320 | 132345483 | 0.001181579 | 33.3877551 | Hyper-methylated | PCa\|NT DMRs |
| chr12 | 132423427 | 132423733 | 0.001181579 | −47.63265306 | Hypo-methylated | PCa\|NT DMRs |
| chr12 | 132663394 | 132663456 | 0.000205174 | −25.64285714 | Hypo-methylated | PCa\|NT DMRs |
| chr12 | 132666489 | 132671672 | 0.000461696 | 27.10140056 | Hyper-methylated | PCa\|NT DMRs |
| chr12 | 132687778 | 132688031 | 0.000688325 | −27.71794872 | Hypo-methylated | PCa\|NT DMRs |
| chr12 | 132688909 | 132689836 | 0.000308889 | −32.90050125 | Hypo-methylated | PCa\|NT DMRs |
| chr12 | 132699875 | 132704243 | 0.000272699 | −25.45468975 | Hypo-methylated | PCa\|NT DMRs |
| chr12 | 132849461 | 132852604 | 1.14E−06 | −21.44015444 | Hypo-methylated | PCa\|NT DMRs |
| chr12 | 132856070 | 132857869 | 0.000323929 | −27.8047619 | Hypo-methylated | PCa\|NT DMRs |
| chr12 | 132922058 | 132924382 | 1.96E−08 | −21.01972789 | Hypo-methylated | PCa\|NT DMRs |
| chr12 | 132934577 | 132934641 | 1.97E−05 | −33.71428571 | Hypo-methylated | PCa\|NT DMRs |
| chr12 | 132938014 | 132940123 | 0.001241245 | −26.88941799 | Hypo-methylated | PCa\|NT DMRs |
| chr12 | 132977135 | 132979726 | 0.004045699 | −24.15833333 | Hypo-methylated | PCa\|NT DMRs |
| chr12 | 132980227 | 132981745 | 0.000754912 | −27.82507937 | Hypo-methylated | PCa\|NT DMRs |
| chr12 | 132991172 | 132991480 | 0.002580892 | −20.46865079 | Hypo-methylated | PCa\|NT DMRs |
| chr12 | 133014314 | 133014429 | 0.001969641 | 31.97619048 | Hyper-methylated | PCa\|NT DMRs |
| chr12 | 133015600 | 133017219 | 0.006225823 | 34.50649351 | Hyper-methylated | PCa\|NT DMRs |
| chr12 | 133021105 | 133021761 | 0.00026034 | 41.33374741 | Hyper-methylated | PCa\|NT DMRs |
| chr12 | 133081803 | 133082783 | 0.004496757 | 23.8358396 | Hyper-methylated | PCa\|NT DMRs |
| chr12 | 133083468 | 133083918 | 2.42E−05 | 27.96984127 | Hyper-methylated | PCa\|NT DMRs |
| chr12 | 133134890 | 133136070 | 2.29E−06 | 39.42792208 | Hyper-methylated | PCa\|NT DMRs |
| chr12 | 133346473 | 133348453 | 5.11E−05 | 43.11339286 | Hyper-methylated | PCa\|NT DMRs |
| chr12 | 133350847 | 133355607 | 3.74E−06 | −22.51949405 | Hypo-methylated | PCa\|NT DMRs |
| chr12 | 133442840 | 133449282 | 0.000822388 | 38.05324675 | Hyper-methylated | PCa\|NT DMRs |
| chr12 | 133458790 | 133459089 | 0.00064571 | −23.48979592 | Hypo-methylated | PCa\|NT DMRs |
| chr12 | 133481352 | 133481529 | 1.21E−06 | 21.42698413 | Hyper-methylated | PCa\|NT DMRs |
| chr13 | 19183558 | 19183583 | 0.007959104 | 25.8 | Hyper-methylated | PCa\|NT DMRs |
| chr13 | 19457406 | 19518336 | 1.52E−09 | −21.17973422 | Hypo-methylated | PCa\|NT DMRs |
| chr13 | 20023259 | 20125651 | 0.000226106 | −32.43040293 | Hypo-methylated | PCa\|NT DMRs |
| chr13 | 20735461 | 20766327 | 5.71E−15 | 28.08456486 | Hyper-methylated | PCa\|NT DMRs |
| chr13 | 21519813 | 21520564 | 3.06E−05 | 33.12205514 | Hyper-methylated | PCa\|NT DMRs |
| chr13 | 21649287 | 21649454 | 0.005968871 | 54.44285714 | Hyper-methylated | PCa\|NT DMRs |
| chr13 | 22408162 | 22494277 | 1.80E−14 | −30.40739796 | Hypo-methylated | PCa\|NT DMRs |
| chr13 | 22502745 | 22551968 | 6.37E−13 | −32.81174603 | Hypo-methylated | PCa\|NT DMRs |
| chr13 | 24289374 | 24289782 | 0.000205174 | −38.58928571 | Hypo-methylated | PCa\|NT DMRs |
| chr13 | 25115530 | 25115729 | 0.002770271 | 23.14285714 | Hyper-methylated | PCa\|NT DMRs |
| chr13 | 25635592 | 25643451 | 4.36E−06 | −28.03781513 | Hypo-methylated | PCa\|NT DMRs |
| chr13 | 27295824 | 27296022 | 5.19E−07 | 34.13772894 | Hyper-methylated | PCa\|NT DMRs |
| chr13 | 27846149 | 27847366 | 0.004677148 | −34.67272727 | Hypo-methylated | PCa\|NT DMRs |
| chr13 | 28318676 | 28318729 | 0.001969641 | −40.63888889 | Hypo-methylated | PCa\|NT DMRs |
| chr13 | 28363625 | 28364738 | 7.87E−12 | 21.6172619 | Hyper-methylated | PCa\|NT DMRs |
| chr13 | 28417350 | 28432473 | 0.000603675 | −26.18392857 | Hypo-methylated | PCa\|NT DMRs |
| chr13 | 28527434 | 28527935 | 1.49E−06 | 24.32640693 | Hyper-methylated | PCa\|NT DMRs |
| chr13 | 28541098 | 28541421 | 0.000159287 | 20.22539683 | Hyper-methylated | PCa\|NT DMRs |
| chr13 | 28944197 | 29060644 | 0.003122338 | −38.9734127 | Hypo-methylated | PCa\|NT DMRs |
| chr13 | 29157973 | 29220757 | 0.001902894 | −25.97321429 | Hypo-methylated | PCa\|NT DMRs |
| chr13 | 30077315 | 30077371 | 0.000974795 | 35.5 | Hyper-methylated | PCa\|NT DMRs |
| chr13 | 30251580 | 30268118 | 0.000972067 | −24.36785714 | Hypo-methylated | PCa\|NT DMRs |
| chr13 | 30681749 | 30681998 | 0.00064571 | 44.32653061 | Hyper-methylated | PCa\|NT DMRs |
| chr13 | 31437071 | 31437213 | 0.005968871 | −51.11428571 | Hypo-methylated | PCa\|NT DMRs |
| chr13 | 31446658 | 31470101 | 0.001254235 | −21.43589744 | Hypo-methylated | PCa\|NT DMRs |
| chr13 | 31480865 | 31481662 | 1.76E−06 | 27.36507937 | Hyper-methylated | PCa\|NT DMRs |
| chr13 | 31573145 | 31595895 | 0.00017871 | −20.84857143 | Hypo-methylated | PCa\|NT DMRs |
| chr13 | 34909982 | 34910047 | 0.005968871 | −40.63809524 | Hypo-methylated | PCa\|NT DMRs |
| chr13 | 37122118 | 37125383 | 0.005968871 | −25.38666667 | Hypo-methylated | PCa\|NT DMRs |
| chr13 | 37729815 | 38159654 | 7.66E−12 | −25.1231685 | Hypo-methylated | PCa\|NT DMRs |
| chr13 | 38307851 | 38402541 | 0.003892841 | −22.10899471 | Hypo-methylated | PCa\|NT DMRs |
| chr13 | 38443104 | 38443480 | 1.92E−05 | 30.28125 | Hyper-methylated | PCa\|NT DMRs |
| chr13 | 39701730 | 39805476 | 0.00068166 | −29.63035714 | Hypo-methylated | PCa\|NT DMRs |
| chr13 | 40402446 | 40410011 | 0.00641359 | −23.6547619 | Hypo-methylated | PCa\|NT DMRs |
| chr13 | 40791366 | 40850557 | 0.002437742 | −25.01216931 | Hypo-methylated | PCa\|NT DMRs |
| chr13 | 44947701 | 44948373 | 9.40E−25 | 23.09492888 | Hyper-methylated | PCa\|NT DMRs |
| chr13 | 44980998 | 44995041 | 0.009510325 | 22.24732143 | Hyper-methylated | PCa\|NT DMRs |
| chr13 | 45493435 | 45495087 | 0.000822388 | −21.85952381 | Hypo-methylated | PCa\|NT DMRs |
| chr13 | 45535113 | 45562412 | 0.005968871 | −28.1352381 | Hypo-methylated | PCa\|NT DMRs |
| chr13 | 46708988 | 46736724 | 0.002770271 | −29.23333333 | Hypo-methylated | PCa\|NT DMRs |
| chr13 | 47392321 | 47447169 | 0.001480644 | −41.76761905 | Hypo-methylated | PCa\|NT DMRs |
| chr13 | 48891926 | 48895324 | 3.37E−30 | 28.2506008 | Hyper-methylated | PCa\|NT DMRs |
| chr13 | 49888148 | 49888351 | 0.001969641 | −50.27380952 | Hypo-methylated | PCa\|NT DMRs |
| chr13 | 50070551 | 50070719 | 1.97E−05 | 40.36428571 | Hyper-methylated | PCa\|NT DMRs |
| chr13 | 50704030 | 50704319 | 0.001429556 | 24.32987013 | Hyper-methylated | PCa\|NT DMRs |
| chr13 | 50706360 | 50708019 | 3.24E−08 | 32.2772381 | Hyper-methylated | PCa\|NT DMRs |
| chr13 | 50901229 | 50940003 | 0.004057568 | −31.94603175 | Hypo-methylated | PCa\|NT DMRs |
| chr13 | 51861176 | 51866825 | 0.00064571 | −27.05782313 | Hypo-methylated | PCa\|NT DMRs |
| chr13 | 52766785 | 52767044 | 0.001969641 | −38.30555556 | Hypo-methylated | PCa\|NT DMRs |
| chr13 | 52918039 | 52918598 | 0.005968871 | −21.38285714 | Hypo-methylated | PCa\|NT DMRs |
| chr13 | 54357656 | 54396152 | 1.76E−06 | −47.01865079 | Hypo-methylated | PCa\|NT DMRs |
| chr13 | 55403634 | 55628877 | 3.58E−08 | −27.47517007 | Hypo-methylated | PCa\|NT DMRs |
| chr13 | 56075445 | 56537800 | 5.35E−12 | −33.41660859 | Hypo-methylated | PCa\|NT DMRs |
| chr13 | 57204589 | 57476539 | 3.97E−12 | −25.51481481 | Hypo-methylated | PCa\|NT DMRs |
| chr13 | 57545059 | 57768939 | 0.000143553 | −28.20714286 | Hypo-methylated | PCa\|NT DMRs |
| chr13 | 57860559 | 57860886 | 0.000117699 | −31.94805195 | Hypo-methylated | PCa\|NT DMRs |
| chr13 | 58368916 | 58826515 | 2.28E−11 | −36.03611111 | Hypo-methylated | PCa\|NT DMRs |
| chr13 | 60841835 | 60842991 | 0.003892841 | 23.17460317 | Hyper-methylated | PCa\|NT DMRs |
| chr13 | 61804141 | 61804730 | 6.42E−05 | −25.87830688 | Hypo-methylated | PCa\|NT DMRs |
| chr13 | 61987935 | 61988042 | 6.42E−05 | 26.06402116 | Hyper-methylated | PCa\|NT DMRs |
| chr13 | 62359010 | 62499812 | 1.37E−09 | −28.52857143 | Hypo-methylated | PCa\|NT DMRs |
| chr13 | 64167770 | 64405061 | 2.89E−14 | −24.96595745 | Hypo-methylated | PCa\|NT DMRs |
| chr13 | 64421684 | 64603893 | 6.90E−06 | −30.17301587 | Hypo-methylated | PCa\|NT DMRs |
| chr13 | 64652771 | 64664705 | 9.62E−06 | −26.44444444 | Hypo-methylated | PCa\|NT DMRs |
| chr13 | 66357827 | 66444160 | 5.44E−07 | −29.2556391 | Hypo-methylated | PCa\|NT DMRs |
| chr13 | 67311776 | 67311966 | 0.001789867 | −28.55343915 | Hypo-methylated | PCa\|NT DMRs |
| chr13 | 67568473 | 67568589 | 0.000205174 | 28.22380952 | Hyper-methylated | PCa\|NT DMRs |
| chr13 | 67974608 | 68349966 | 1.47E−11 | −33.18893557 | Hypo-methylated | PCa\|NT DMRs |
| chr13 | 71546030 | 71724087 | 0.001969641 | −31.06394558 | Hypo-methylated | PCa\|NT DMRs |
| chr13 | 74666532 | 74708285 | 5.00E−07 | 24.14506803 | Hyper-methylated | PCa\|NT DMRs |
| chr13 | 76209395 | 76209621 | 8.73E−05 | −31.9744898 | Hypo-methylated | PCa\|NT DMRs |
| chr13 | 78117063 | 78132808 | 0.001181579 | −39.33333333 | Hypo-methylated | PCa\|NT DMRs |
| chr13 | 79284992 | 79942284 | 6.48E−16 | −30.59349962 | Hypo-methylated | PCa\|NT DMRs |
| chr13 | 80426582 | 80823358 | 4.24E−17 | −31.77419913 | Hypo-methylated | PCa\|NT DMRs |
| chr13 | 80937371 | 81198124 | 4.62E−14 | −33.21168831 | Hypo-methylated | PCa\|NT DMRs |
| chr13 | 81324988 | 81600596 | 1.47E−07 | −29.12212885 | Hypo-methylated | PCa\|NT DMRs |
| chr13 | 82088183 | 82308857 | 2.81E−09 | −29.90095238 | Hypo-methylated | PCa\|NT DMRs |
| chr13 | 84480709 | 84610861 | 1.43E−08 | −29.08456486 | Hypo-methylated | PCa\|NT DMRs |
| chr13 | 85058581 | 85058635 | 0.000974795 | −31.66369048 | Hypo-methylated | PCa\|NT DMRs |
| chr13 | 85470198 | 85536515 | 2.73E−08 | −29.34927536 | Hypo-methylated | PCa\|NT DMRs |
| chr13 | 86427492 | 86498929 | 6.56E−06 | −29.73730159 | Hypo-methylated | PCa\|NT DMRs |
| chr13 | 86590986 | 87420902 | 4.90E−14 | −29.40592992 | Hypo-methylated | PCa\|NT DMRs |
| chr13 | 87490771 | 87542596 | 0.00051204 | −25.11190476 | Hypo-methylated | PCa\|NT DMRs |
| chr13 | 88435008 | 88600195 | 9.35E−06 | −29.79546485 | Hypo-methylated | PCa\|NT DMRs |
| chr13 | 90026614 | 90317749 | 1.87E−16 | −29.87725948 | Hypo-methylated | PCa\|NT DMRs |
| chr13 | 93744085 | 93748882 | 0.000172237 | −23.98707483 | Hypo-methylated | PCa\|NT DMRs |
| chr13 | 95620295 | 95719742 | 5.56E−20 | 26.89817276 | Hyper-methylated | PCa\|NT DMRs |
| chr13 | 95845546 | 95855253 | 0.003305567 | −22.65833333 | Hypo-methylated | PCa\|NT DMRs |
| chr13 | 96294544 | 96296438 | 2.16E−06 | 22.59863946 | Hyper-methylated | PCa\|NT DMRs |
| chr13 | 98866102 | 98866483 | 0.000554289 | −30.99206349 | Hypo-methylated | PCa\|NT DMRs |
| chr13 | 99057348 | 99058218 | 0.001969641 | −37.11904762 | Hypo-methylated | PCa\|NT DMRs |
| chr13 | 99064004 | 99064193 | 0.008482405 | 50.71428571 | Hyper-methylated | PCa\|NT DMRs |
| chr13 | 99083366 | 99083678 | 6.94E−06 | −45.36952381 | Hypo-methylated | PCa\|NT DMRs |
| chr13 | 100310212 | 100310842 | 7.47E−06 | 24.45238095 | Hyper-methylated | PCa\|NT DMRs |
| chr13 | 100568076 | 100578586 | 0.001969641 | −40.21238095 | Hypo-methylated | PCa\|NT DMRs |
| chr13 | 100624234 | 100630385 | 1.99E−12 | 34.40357143 | Hyper-methylated | PCa\|NT DMRs |
| chr13 | 100740393 | 100740481 | 0.001969641 | −56.45634921 | Hypo-methylated | PCa\|NT DMRs |
| chr13 | 101173580 | 101174097 | 5.20E−08 | 27.04761905 | Hyper-methylated | PCa\|NT DMRs |
| chr13 | 101186116 | 101197410 | 1.42E−05 | −45.65306122 | Hypo-methylated | PCa\|NT DMRs |
| chr13 | 102431388 | 102532842 | 0.000354065 | −31.2026455 | Hypo-methylated | PCa\|NT DMRs |
| chr13 | 102580006 | 102888822 | 1.57E−10 | −32.75416667 | Hypo-methylated | PCa\|NT DMRs |
| chr13 | 102892803 | 103042408 | 6.17E−06 | −37.39087302 | Hypo-methylated | PCa\|NT DMRs |
| chr13 | 103088596 | 103203949 | 1.76E−06 | −42.80952381 | Hypo-methylated | PCa\|NT DMRs |
| chr13 | 103344517 | 103366455 | 2.42E−05 | −26.54931973 | Hypo-methylated | PCa\|NT DMRs |
| chr13 | 106062676 | 106062754 | 6.42E−05 | −23.06137566 | Hypo-methylated | PCa\|NT DMRs |
| chr13 | 107069338 | 107069987 | 0.002373155 | 36.31481481 | Hyper-methylated | PCa\|NT DMRs |
| chr13 | 107799764 | 108506793 | 5.09E−20 | −31.1021164 | Hypo-methylated | PCa\|NT DMRs |
| chr13 | 108535715 | 108831780 | 4.54E−14 | −34.68590865 | Hypo-methylated | PCa\|NT DMRs |
| chr13 | 108922481 | 108922544 | 0.001969641 | 33.88095238 | Hyper-methylated | PCa\|NT DMRs |
| chr13 | 109148894 | 109148956 | 1.97E−05 | 22.91428571 | Hyper-methylated | PCa\|NT DMRs |
| chr13 | 110512413 | 110522309 | 9.48E−07 | 25.31972789 | Hyper-methylated | PCa\|NT DMRs |
| chr13 | 110781664 | 110784041 | 2.12E−05 | −29.52095238 | Hypo-methylated | PCa\|NT DMRs |
| chr13 | 110831650 | 110835962 | 9.20E−10 | −28.10568356 | Hypo-methylated | PCa\|NT DMRs |
| chr13 | 110958690 | 110958946 | 0.000117699 | 23.66233766 | Hyper-methylated | PCa\|NT DMRs |
| chr13 | 111070350 | 111072546 | 0.00064571 | −28.77959184 | Hypo-methylated | PCa\|NT DMRs |
| chr13 | 111106961 | 111107586 | 5.99E−05 | −29.33919414 | Hypo-methylated | PCa\|NT DMRs |
| chr13 | 111159575 | 111160475 | 0.001310211 | −46.35714286 | Hypo-methylated | PCa\|NT DMRs |
| chr13 | 111179796 | 111180080 | 0.001969641 | −25.8531746 | Hypo-methylated | PCa\|NT DMRs |
| chr13 | 111268715 | 111278546 | 9.54E−05 | −36.89409524 | Hypo-methylated | PCa\|NT DMRs |
| chr13 | 111316634 | 111318855 | 3.28E−05 | 21.03380952 | Hyper-methylated | PCa\|NT DMRs |
| chr13 | 111445615 | 111466230 | 9.06E−06 | 25.47261905 | Hyper-methylated | PCa\|NT DMRs |
| chr13 | 111684037 | 111697266 | 6.42E−05 | −24.94444444 | Hypo-methylated | PCa\|NT DMRs |
| chr13 | 111718993 | 111719267 | 5.71E−06 | 40.0264881 | Hyper-methylated | PCa\|NT DMRs |
| chr13 | 112061914 | 112062903 | 2.85E−05 | −38.93040293 | Hypo-methylated | PCa\|NT DMRs |
| chr13 | 112063644 | 112113123 | 5.72E−07 | −21.59313037 | Hypo-methylated | PCa\|NT DMRs |
| chr13 | 112278837 | 112285475 | 1.97E−05 | −54.23619048 | Hypo-methylated | PCa\|NT DMRs |
| chr13 | 112323193 | 112326118 | 1.42E−05 | −26.15986395 | Hypo-methylated | PCa\|NT DMRs |
| chr13 | 112329989 | 112330268 | 0.001969641 | 35.67063492 | Hyper-methylated | PCa\|NT DMRs |
| chr13 | 112612375 | 112630338 | 1.11E−11 | −28.10482625 | Hypo-methylated | PCa\|NT DMRs |
| chr13 | 112630698 | 112706654 | 5.88E−18 | −28.87562657 | Hypo-methylated | PCa\|NT DMRs |
| chr13 | 112837680 | 112837744 | 0.005968871 | 33.91428571 | Hyper-methylated | PCa\|NT DMRs |
| chr13 | 112996346 | 112997267 | 0.000271988 | −22.75714286 | Hypo-methylated | PCa\|NT DMRs |
| chr13 | 112997608 | 113000429 | 0.000406444 | −24.71142857 | Hypo-methylated | PCa\|NT DMRs |
| chr13 | 113011051 | 113018381 | 0.001969641 | −37.26190476 | Hypo-methylated | PCa\|NT DMRs |
| chr13 | 113079481 | 113099478 | 0.005892073 | −21.17891156 | Hypo-methylated | PCa\|NT DMRs |
| chr13 | 113528544 | 113528678 | 3.61E−05 | 38.41836735 | Hyper-methylated | PCa\|NT DMRs |
| chr13 | 113547154 | 113547247 | 0.002086072 | 23.58333333 | Hyper-methylated | PCa\|NT DMRs |
| chr13 | 113556662 | 113557493 | 0.000260936 | −28.44952381 | Hypo-methylated | PCa\|NT DMRs |
| chr13 | 113641885 | 113642543 | 0.008908019 | 27.65714286 | Hyper-methylated | PCa\|NT DMRs |
| chr13 | 113689112 | 113689801 | 0.004095673 | −27.67857143 | Hypo-methylated | PCa\|NT DMRs |
| chr13 | 113765274 | 113765398 | 8.80E−08 | 27.18452381 | Hyper-methylated | PCa\|NT DMRs |
| chr13 | 113807311 | 113807971 | 2.87E−10 | 31.33791887 | Hyper-methylated | PCa\|NT DMRs |
| chr13 | 113979880 | 113980394 | 4.18E−09 | −39.16666667 | Hypo-methylated | PCa\|NT DMRs |
| chr13 | 114055864 | 114056576 | 3.64E−05 | −24.68073593 | Hypo-methylated | PCa\|NT DMRs |
| chr13 | 114064623 | 114065183 | 3.10E−09 | −31.97845805 | Hypo-methylated | PCa\|NT DMRs |
| chr13 | 114075216 | 114075871 | 0.00208105 | −20.45378151 | Hypo-methylated | PCa\|NT DMRs |
| chr13 | 114091590 | 114091770 | 0.004095673 | −46.3531746 | Hypo-methylated | PCa\|NT DMRs |
| chr13 | 114102901 | 114105415 | 0.000117699 | −36.65021645 | Hypo-methylated | PCa\|NT DMRs |
| chr13 | 114145943 | 114146233 | 0.001083007 | 28.79285714 | Hyper-methylated | PCa\|NT DMRs |
| chr13 | 114184420 | 114184510 | 0.001969641 | 40.02380952 | Hyper-methylated | PCa\|NT DMRs |
| chr13 | 114214648 | 114215140 | 0.000510004 | −21.59714286 | Hypo-methylated | PCa\|NT DMRs |
| chr13 | 114429454 | 114429609 | 0.009382828 | −25.04232804 | Hypo-methylated | PCa\|NT DMRs |
| chr13 | 114439188 | 114439618 | 0.003391914 | 27.94497354 | Hyper-methylated | PCa\|NT DMRs |
| chr13 | 114450316 | 114450423 | 0.000145094 | 29.40595238 | Hyper-methylated | PCa\|NT DMRs |
| chr13 | 114467102 | 114467359 | 5.94E−06 | −26.09307359 | Hypo-methylated | PCa\|NT DMRs |
| chr13 | 114496122 | 114496429 | 0.005513661 | −33.00952381 | Hypo-methylated | PCa\|NT DMRs |
| chr13 | 114507064 | 114507393 | 3.05E−06 | −20.9047619 | Hypo-methylated | PCa\|NT DMRs |
| chr13 | 114514329 | 114515665 | 8.83E−06 | −26.17432712 | Hypo-methylated | PCa\|NT DMRs |
| chr13 | 114515714 | 114518296 | 1.37E−06 | −28.7032967 | Hypo-methylated | PCa\|NT DMRs |
| chr13 | 114519669 | 114519709 | 0.00179283 | 40.26964286 | Hyper-methylated | PCa\|NT DMRs |
| chr13 | 114519915 | 114520039 | 0.00641359 | −23.86111111 | Hypo-methylated | PCa\|NT DMRs |
| chr13 | 114549606 | 114549853 | 0.001969641 | 34.61564626 | Hyper-methylated | PCa\|NT DMRs |
| chr13 | 114559758 | 114559971 | 0.005968871 | −63.02857143 | Hypo-methylated | PCa\|NT DMRs |
| chr13 | 114565894 | 114566687 | 1.76E−12 | 27.95772595 | Hyper-methylated | PCa\|NT DMRs |
| chr13 | 114740004 | 114740618 | 3.42E−06 | −57.12946429 | Hypo-methylated | PCa\|NT DMRs |
| chr13 | 114764602 | 114765478 | 0.000632596 | −21.68571429 | Hypo-methylated | PCa\|NT DMRs |
| chr13 | 114826043 | 114826549 | 7.78E−09 | −24.20851371 | Hypo-methylated | PCa\|NT DMRs |
| chr13 | 114828436 | 114829497 | 0.001985874 | 41.84619048 | Hyper-methylated | PCa\|NT DMRs |
| chr13 | 114831687 | 114834923 | 7.06E−07 | 30.8265873 | Hyper-methylated | PCa\|NT DMRs |
| chr13 | 114847300 | 114847659 | 0.001969641 | −42.60204082 | Hypo-methylated | PCa\|NT DMRs |
| chr13 | 114875869 | 114876391 | 3.92E−06 | 38.03896104 | Hyper-methylated | PCa\|NT DMRs |
| chr13 | 114894088 | 114897135 | 0.008243355 | 22.02298137 | Hyper-methylated | PCa\|NT DMRs |
| chr14 | 20898057 | 20898224 | 1.97E−05 | 47.77619048 | Hyper-methylated | PCa\|NT DMRs |
| chr14 | 21069688 | 21069928 | 1.76E−06 | 35.69047619 | Hyper-methylated | PCa\|NT DMRs |
| chr14 | 21091062 | 21094158 | 3.69E−05 | 26.62545156 | Hyper-methylated | PCa\|NT DMRs |
| chr14 | 21979872 | 21983833 | 0.004178506 | −26.5327381 | Hypo-methylated | PCa\|NT DMRs |
| chr14 | 23270004 | 23270131 | 0.001181579 | 38.89591837 | Hyper-methylated | PCa\|NT DMRs |
| chr14 | 23276073 | 23290620 | 0.005513661 | −21.32916667 | Hypo-methylated | PCa\|NT DMRs |
| chr14 | 23583383 | 23588172 | 7.86E−08 | 28.79858907 | Hyper-methylated | PCa\|NT DMRs |
| chr14 | 23611367 | 23623810 | 0.00064571 | −38.03174603 | Hypo-methylated | PCa\|NT DMRs |
| chr14 | 23821229 | 23821446 | 3.05E−06 | 33.53479853 | Hyper-methylated | PCa\|NT DMRs |
| chr14 | 23834789 | 23835756 | 4.47E−09 | 32.95767196 | Hyper-methylated | PCa\|NT DMRs |
| chr14 | 24540025 | 24540415 | 0.005513661 | 28.04081633 | Hyper-methylated | PCa\|NT DMRs |
| chr14 | 24780434 | 24780905 | 4.17E−06 | 30.66265664 | Hyper-methylated | PCa\|NT DMRs |
| chr14 | 24808422 | 24809179 | 1.92E−12 | 38.33552381 | Hyper-methylated | PCa\|NT DMRs |
| chr14 | 24837540 | 24838170 | 3.97E−13 | 34.00069686 | Hyper-methylated | PCa\|NT DMRs |
| chr14 | 24951105 | 24951258 | 0.005968871 | 44.77142857 | Hyper-methylated | PCa\|NT DMRs |
| chr14 | 26725518 | 27062728 | 1.29E−07 | −35.31746032 | Hypo-methylated | PCa\|NT DMRs |
| chr14 | 29772983 | 29952738 | 0.001076343 | −34.7978355 | Hypo-methylated | PCa\|NT DMRs |
| chr14 | 30646231 | 30791276 | 0.000783791 | −35.39380952 | Hypo-methylated | PCa\|NT DMRs |
| chr14 | 30893763 | 30897658 | 0.005968871 | −26.72952381 | Hypo-methylated | PCa\|NT DMRs |
| chr14 | 31159737 | 31159787 | 0.005968871 | −22.45714286 | Hypo-methylated | PCa\|NT DMRs |
| chr14 | 31344151 | 31345058 | 0.000157373 | 25.3452381 | Hyper-methylated | PCa\|NT DMRs |
| chr14 | 32950619 | 32979137 | 0.00051204 | −50.03571429 | Hypo-methylated | PCa\|NT DMRs |
| chr14 | 34268920 | 34270337 | 6.88E−16 | 26.18995671 | Hyper-methylated | PCa\|NT DMRs |
| chr14 | 34418609 | 34419838 | 0.000339627 | 23.91119048 | Hyper-methylated | PCa\|NT DMRs |
| chr14 | 35020778 | 35021286 | 0.001181579 | −24.65170068 | Hypo-methylated | PCa\|NT DMRs |
| chr14 | 38678648 | 38680856 | 5.59E−12 | 30.71679198 | Hyper-methylated | PCa\|NT DMRs |
| chr14 | 40933321 | 41599372 | 2.95E−13 | −28.12945326 | Hypo-methylated | PCa\|NT DMRs |
| chr14 | 41878730 | 42040754 | 0.003159484 | −25.34603175 | Hypo-methylated | PCa\|NT DMRs |
| chr14 | 42074561 | 42075407 | 4.19E−06 | 26.5978836 | Hyper-methylated | PCa\|NT DMRs |
| chr14 | 43046504 | 43817028 | 9.21E−23 | −28.41696429 | Hypo-methylated | PCa\|NT DMRs |
| chr14 | 44854012 | 45221580 | 2.68E−17 | −27.98486395 | Hypo-methylated | PCa\|NT DMRs |
| chr14 | 45268046 | 45343117 | 0.000176046 | −29.1004329 | Hypo-methylated | PCa\|NT DMRs |
| chr14 | 48096085 | 48142684 | 1.46E−10 | −30.18301158 | Hypo-methylated | PCa\|NT DMRs |
| chr14 | 50155167 | 50155331 | 0.000396159 | 29.18428571 | Hyper-methylated | PCa\|NT DMRs |
| chr14 | 51251974 | 51252499 | 0.009461608 | 24.81309524 | Hyper-methylated | PCa\|NT DMRs |
| chr14 | 51410501 | 51410922 | 4.79E−06 | 24.05416667 | Hyper-methylated | PCa\|NT DMRs |
| chr14 | 51974848 | 51975014 | 0.00023371 | 41.67532468 | Hyper-methylated | PCa\|NT DMRs |
| chr14 | 52218930 | 52219392 | 5.42E−05 | −36.96825397 | Hypo-methylated | PCa\|NT DMRs |
| chr14 | 52509589 | 52518560 | 0.007959104 | −37.26394558 | Hypo-methylated | PCa\|NT DMRs |
| chr14 | 54686140 | 54687028 | 3.85E−08 | 22.74421769 | Hyper-methylated | PCa\|NT DMRs |
| chr14 | 54732013 | 54810795 | 1.43E−05 | −30.59642857 | Hypo-methylated | PCa\|NT DMRs |
| chr14 | 55120606 | 55120861 | 0.009503366 | 34.15357143 | Hyper-methylated | PCa\|NT DMRs |
| chr14 | 55583322 | 55596561 | 6.41E−23 | 22.535 | Hyper-methylated | PCa\|NT DMRs |
| chr14 | 55907199 | 55907375 | 1.24E−05 | 24.73361345 | Hyper-methylated | PCa\|NT DMRs |
| chr14 | 56471819 | 56471887 | 0.005968871 | 22.65714286 | Hyper-methylated | PCa\|NT DMRs |
| chr14 | 58484871 | 58593799 | 0.006976128 | −25.47703081 | Hypo-methylated | PCa\|NT DMRs |
| chr14 | 60043077 | 60043556 | 4.20E−06 | 20.7443609 | Hyper-methylated | PCa\|NT DMRs |
| chr14 | 60794382 | 60795094 | 9.06E−13 | 21.71138211 | Hyper-methylated | PCa\|NT DMRs |
| chr14 | 61032090 | 61032402 | 0.005968871 | −44.86666667 | Hypo-methylated | PCa\|NT DMRs |
| chr14 | 61118385 | 61119886 | 7.00E−11 | 39.48065476 | Hyper-methylated | PCa\|NT DMRs |
| chr14 | 61654904 | 61655229 | 0.00068166 | 24.5031746 | Hyper-methylated | PCa\|NT DMRs |
| chr14 | 62035355 | 62035512 | 1.35E−09 | 38.19047619 | Hyper-methylated | PCa\|NT DMRs |
| chr14 | 64208014 | 64215847 | 0.000333533 | −40.14319728 | Hypo-methylated | PCa\|NT DMRs |
| chr14 | 64580144 | 64580232 | 0.005968871 | −59.25714286 | Hypo-methylated | PCa\|NT DMRs |
| chr14 | 65007847 | 65009126 | 8.08E−07 | 29.63251232 | Hyper-methylated | PCa\|NT DMRs |
| chr14 | 65172415 | 65172612 | 5.94E−06 | 50.66233766 | Hyper-methylated | PCa\|NT DMRs |
| chr14 | 65186709 | 65188402 | 6.42E−05 | −32.06349206 | Hypo-methylated | PCa\|NT DMRs |
| chr14 | 65438225 | 65438378 | 0.001115302 | 26.85416667 | Hyper-methylated | PCa\|NT DMRs |
| chr14 | 65749247 | 65754508 | 0.000115085 | 26.89223058 | Hyper-methylated | PCa\|NT DMRs |
| chr14 | 67955189 | 67955474 | 0.001969641 | 52.78571429 | Hyper-methylated | PCa\|NT DMRs |
| chr14 | 68283984 | 68283999 | 0.008482405 | 46.66190476 | Hyper-methylated | PCa\|NT DMRs |
| chr14 | 68750407 | 68750507 | 0.005968871 | 55.28095238 | Hyper-methylated | PCa\|NT DMRs |
| chr14 | 69095026 | 69095336 | 9.06E−13 | 33.7358885 | Hyper-methylated | PCa\|NT DMRs |
| chr14 | 69158048 | 69170010 | 0.001907648 | 23.45952381 | Hyper-methylated | PCa\|NT DMRs |
| chr14 | 69395207 | 69396145 | 0.005438982 | 29.3047619 | Hyper-methylated | PCa\|NT DMRs |
| chr14 | 69402596 | 69403049 | 0.003871035 | −24.16071429 | Hypo-methylated | PCa\|NT DMRs |
| chr14 | 69813636 | 69813782 | 0.001969641 | 40.07142857 | Hyper-methylated | PCa\|NT DMRs |
| chr14 | 70039398 | 70040020 | 1.54E−18 | 22.77529412 | Hyper-methylated | PCa\|NT DMRs |
| chr14 | 70125243 | 70125430 | 0.005968871 | −49.28571429 | Hypo-methylated | PCa\|NT DMRs |
| chr14 | 70653765 | 70654671 | 3.23E−07 | 21.43537415 | Hyper-methylated | PCa\|NT DMRs |
| chr14 | 70756046 | 70756121 | 0.000205174 | 43.43452381 | Hyper-methylated | PCa\|NT DMRs |
| chr14 | 71583517 | 71583703 | 0.005968871 | −43.2 | Hypo-methylated | PCa\|NT DMRs |
| chr14 | 72239539 | 72318581 | 3.74E−06 | −26.53869048 | Hypo-methylated | PCa\|NT DMRs |
| chr14 | 72357011 | 72375125 | 0.005968871 | −37.61428571 | Hypo-methylated | PCa\|NT DMRs |
| chr14 | 72425642 | 72938442 | 8.50E−20 | −31.36443641 | Hypo-methylated | PCa\|NT DMRs |
| chr14 | 72946891 | 73072480 | 3.29E−12 | −30.92325397 | Hypo-methylated | PCa\|NT DMRs |
| chr14 | 73082376 | 73177392 | 1.65E−19 | −37.25628816 | Hypo-methylated | PCa\|NT DMRs |
| chr14 | 74177956 | 74180312 | 0.000487946 | 33.75666667 | Hyper-methylated | PCa\|NT DMRs |
| chr14 | 74409083 | 74409329 | 0.007959104 | 28.85714286 | Hyper-methylated | PCa\|NT DMRs |
| chr14 | 74438909 | 74462826 | 0.000494377 | −26.59727891 | Hypo-methylated | PCa\|NT DMRs |
| chr14 | 74725151 | 74725348 | 0.003032307 | 27.21972789 | Hyper-methylated | PCa\|NT DMRs |
| chr14 | 74769976 | 74770137 | 0.004947537 | −27.86772487 | Hypo-methylated | PCa\|NT DMRs |
| chr14 | 75342756 | 75342882 | 0.005968871 | 40.13809524 | Hyper-methylated | PCa\|NT DMRs |
| chr14 | 75408511 | 75408957 | 0.003391914 | 37.43544974 | Hyper-methylated | PCa\|NT DMRs |
| chr14 | 75800254 | 75801077 | 1.02E−09 | 51.41878307 | Hyper-methylated | PCa\|NT DMRs |
| chr14 | 75893926 | 75894395 | 1.63E−07 | 25.11520737 | Hyper-methylated | PCa\|NT DMRs |
| chr14 | 76006589 | 76009568 | 0.007560755 | 28.16320346 | Hyper-methylated | PCa\|NT DMRs |
| chr14 | 76600683 | 76600792 | 0.000481535 | −27.38994709 | Hypo-methylated | PCa\|NT DMRs |
| chr14 | 76939917 | 76940337 | 0.002534928 | 21.31666667 | Hyper-methylated | PCa\|NT DMRs |
| chr14 | 77422419 | 77422738 | 0.003892896 | 43.81428571 | Hyper-methylated | PCa\|NT DMRs |
| chr14 | 77428060 | 77428383 | 4.28E−07 | 20.27969925 | Hyper-methylated | PCa\|NT DMRs |
| chr14 | 77434995 | 77435580 | 0.003314163 | −35.95714286 | Hypo-methylated | PCa\|NT DMRs |
| chr14 | 77489712 | 77493458 | 3.96E−32 | −49.91744792 | Hypo-methylated | PCa\|NT DMRs |
| chr14 | 78432683 | 78432797 | 0.000215387 | 31.78571429 | Hyper-methylated | PCa\|NT DMRs |
| chr14 | 79111674 | 79111760 | 0.004095673 | −22.5 | Hypo-methylated | PCa\|NT DMRs |
| chr14 | 79746628 | 79746781 | 0.000205174 | 22.07440476 | Hyper-methylated | PCa\|NT DMRs |
| chr14 | 81433721 | 81439725 | 0.002141488 | −32.32380952 | Hypo-methylated | PCa\|NT DMRs |
| chr14 | 82296042 | 82478330 | 3.47E−05 | −30.86412698 | Hypo-methylated | PCa\|NT DMRs |
| chr14 | 83515420 | 83666727 | 1.38E−13 | −30.69813259 | Hypo-methylated | PCa\|NT DMRs |
| chr14 | 83703627 | 84283285 | 3.64E−17 | −32.36137566 | Hypo-methylated | PCa\|NT DMRs |
| chr14 | 84484112 | 84484266 | 0.001969641 | −45.95918367 | Hypo-methylated | PCa\|NT DMRs |
| chr14 | 89493240 | 89493318 | 0.00015047 | 33.56043956 | Hyper-methylated | PCa\|NT DMRs |
| chr14 | 89631058 | 89644632 | 0.007959104 | 26.33265306 | Hyper-methylated | PCa\|NT DMRs |
| chr14 | 89771438 | 89771499 | 0.005968871 | −29.03809524 | Hypo-methylated | PCa\|NT DMRs |
| chr14 | 89818887 | 89837830 | 0.003034084 | −22.64795918 | Hypo-methylated | PCa\|NT DMRs |
| chr14 | 89977035 | 89977212 | 0.001969641 | 29.90873016 | Hyper-methylated | PCa\|NT DMRs |
| chr14 | 90967174 | 90967644 | 1.27E−08 | 45.66755952 | Hyper-methylated | PCa\|NT DMRs |
| chr14 | 91287431 | 91287580 | 0.004095673 | 44.5 | Hyper-methylated | PCa\|NT DMRs |
| chr14 | 91731083 | 91731425 | 0.006185293 | 23.37414966 | Hyper-methylated | PCa\|NT DMRs |
| chr14 | 91740414 | 91740866 | 5.52E−05 | 28.66233766 | Hyper-methylated | PCa\|NT DMRs |
| chr14 | 91743406 | 91744282 | 0.001969641 | −35.42857143 | Hypo-methylated | PCa\|NT DMRs |
| chr14 | 91860314 | 91862979 | 0.007563547 | −25.54545455 | Hypo-methylated | PCa\|NT DMRs |
| chr14 | 91881488 | 91881788 | 6.42E−05 | 36.22751323 | Hyper-methylated | PCa\|NT DMRs |
| chr14 | 92413633 | 92413768 | 1.35E−05 | 31.30340136 | Hyper-methylated | PCa\|NT DMRs |
| chr14 | 92652091 | 92652216 | 0.001969641 | 29.12936508 | Hyper-methylated | PCa\|NT DMRs |
| chr14 | 92680323 | 92691421 | 0.003391914 | −21.37460317 | Hypo-methylated | PCa\|NT DMRs |
| chr14 | 92790439 | 92790508 | 9.41E−05 | 25.56410256 | Hyper-methylated | PCa\|NT DMRs |
| chr14 | 92849470 | 92850047 | 0.000260936 | −21.09333333 | Hypo-methylated | PCa\|NT DMRs |
| chr14 | 92853140 | 92853227 | 0.005968871 | 49.81904762 | Hyper-methylated | PCa\|NT DMRs |
| chr14 | 92980747 | 92981143 | 4.21E−07 | 27.4141369 | Hyper-methylated | PCa\|NT DMRs |
| chr14 | 93006310 | 93010077 | 0.001181579 | 35.11904762 | Hyper-methylated | PCa\|NT DMRs |
| chr14 | 93115555 | 93125829 | 1.07E−06 | 39.0385034 | Hyper-methylated | PCa\|NT DMRs |
| chr14 | 93153250 | 93154607 | 1.87E−28 | −44.42589893 | Hypo-methylated | PCa\|NT DMRs |
| chr14 | 93389695 | 93389744 | 0.000143553 | 20.52312925 | Hyper-methylated | PCa\|NT DMRs |
| chr14 | 93412555 | 93413112 | 0.002044625 | −43.60822511 | Hypo-methylated | PCa\|NT DMRs |
| chr14 | 93505152 | 93512934 | 0.002097193 | 31.20068027 | Hyper-methylated | PCa\|NT DMRs |
| chr14 | 93827416 | 93885481 | 6.42E−05 | −27.68518519 | Hypo-methylated | PCa\|NT DMRs |
| chr14 | 93925461 | 93968616 | 0.000122432 | −25.78354978 | Hypo-methylated | PCa\|NT DMRs |
| chr14 | 94032483 | 94084304 | 5.86E−11 | −23.36816327 | Hypo-methylated | PCa\|NT DMRs |
| chr14 | 94253752 | 94254842 | 1.66E−11 | 31.65 | Hyper-methylated | PCa\|NT DMRs |
| chr14 | 94291032 | 94291414 | 0.001451132 | −21.89010989 | Hypo-methylated | PCa\|NT DMRs |
| chr14 | 94360021 | 94360328 | 0.001969641 | −32.01020408 | Hypo-methylated | PCa\|NT DMRs |
| chr14 | 94467340 | 94470596 | 7.48E−05 | −34.83915344 | Hypo-methylated | PCa\|NT DMRs |
| chr14 | 95237574 | 95244825 | 1.60E−19 | 34.16367521 | Hyper-methylated | PCa\|NT DMRs |
| chr14 | 95403215 | 95403422 | 5.42E−05 | −27.74246032 | Hypo-methylated | PCa\|NT DMRs |
| chr14 | 95837370 | 95837624 | 3.66E−05 | 36.95047619 | Hyper-methylated | PCa\|NT DMRs |
| chr14 | 95882539 | 95898749 | 0.005295761 | −36.07272727 | Hypo-methylated | PCa\|NT DMRs |
| chr14 | 95966492 | 95966917 | 0.000847246 | −31.85457875 | Hypo-methylated | PCa\|NT DMRs |
| chr14 | 96240765 | 96341008 | 0.00023938 | −24.58853616 | Hypo-methylated | PCa\|NT DMRs |
| chr14 | 96362466 | 96428783 | 2.37E−05 | −33.07460317 | Hypo-methylated | PCa\|NT DMRs |
| chr14 | 96520635 | 96521554 | 0.000205174 | −27.15 | Hypo-methylated | PCa\|NT DMRs |
| chr14 | 96707164 | 96728062 | 5.52E−05 | −23.4914966 | Hypo-methylated | PCa\|NT DMRs |
| chr14 | 96730215 | 96730456 | 0.00064571 | −49.34693878 | Hypo-methylated | PCa\|NT DMRs |
| chr14 | 96735546 | 96735868 | 0.002767976 | −20.94805195 | Hypo-methylated | PCa\|NT DMRs |
| chr14 | 97093420 | 97093518 | 0.005968871 | 38.58952381 | Hyper-methylated | PCa\|NT DMRs |
| chr14 | 97399081 | 97463882 | 6.29E−12 | −22.4707672 | Hypo-methylated | PCa\|NT DMRs |
| chr14 | 97492461 | 97494253 | 0.00064571 | −45.04421769 | Hypo-methylated | PCa\|NT DMRs |
| ehrl4 | 97556874 | 97683619 | 7.86E−08 | −37.4287415 | Hypo-methylated | PCa\|NT DMRs |
| chr14 | 97693791 | 98178276 | 1.27E−28 | −36.62623907 | Hypo-methylated | PCa\|NT DMRs |
| chr14 | 98224071 | 98455419 | 1.69E−13 | −34.33189369 | Hypo-methylated | PCa\|NT DMRs |
| chr14 | 99420764 | 99499551 | 4.18E−14 | −29.09435626 | Hypo-methylated | PCa\|NT DMRs |
| chr14 | 99506229 | 99506674 | 1.42E−05 | −31.6292517 | Hypo-methylated | PCa\|NT DMRs |
| chr14 | 99532758 | 99532894 | 1.76E−06 | −34.33333333 | Hypo-methylated | PCa\|NT DMRs |
| chr14 | 99658816 | 99658939 | 0.001969641 | −33.87482993 | Hypo-methylated | PCa\|NT DMRs |
| chr14 | 99711875 | 99711921 | 0.001969641 | 28.02777778 | Hyper-methylated | PCa\|NT DMRs |
| chr14 | 100039827 | 100043609 | 0.000807276 | −35.15546218 | Hypo-methylated | PCa\|NT DMRs |
| chr14 | 100091467 | 100091521 | 0.000377317 | −31.11607143 | Hypo-methylated | PCa\|NT DMRs |
| chr14 | 100125594 | 100130466 | 1.97E−06 | 27.67953394 | Hyper-methylated | PCa\|NT DMRs |
| chr14 | 100133204 | 100146451 | 2.99E−05 | 22.55868726 | Hyper-methylated | PCa\|NT DMRs |
| chr14 | 100148295 | 100148363 | 0.005968871 | −35.13333333 | Hypo-methylated | PCa\|NT DMRs |
| chr14 | 100154773 | 100192812 | 0.001975917 | 25.26479076 | Hyper-methylated | PCa\|NT DMRs |
| chr14 | 100201160 | 100201236 | 0.001969641 | −44 | Hypo-methylated | PCa\|NT DMRs |
| chr14 | 100212986 | 100213394 | 0.000570576 | 25.57936508 | Hyper-methylated | PCa\|NT DMRs |
| chr14 | 100216292 | 100216603 | 0.006589535 | −28.68571429 | Hypo-methylated | PCa\|NT DMRs |
| chr14 | 100431319 | 100437027 | 0.000494377 | −42.68061224 | Hypo-methylated | PCa\|NT DMRs |
| chr14 | 100496719 | 100537036 | 0.005968871 | −34.8 | Hypo-methylated | PCa\|NT DMRs |
| chr14 | 100621949 | 100622582 | 5.84E−05 | −51.34960317 | Hypo-methylated | PCa\|NT DMRs |
| chr14 | 100784369 | 100784847 | 5.86E−07 | 22.70539683 | Hyper-methylated | PCa\|NT DMRs |
| chr14 | 100859981 | 100883064 | 0.000132829 | 29.51753247 | Hyper-methylated | PCa\|NT DMRs |
| chr14 | 100907114 | 100907317 | 0.005968871 | −23.8 | Hypo-methylated | PCa\|NT DMRs |
| chr14 | 100998266 | 101003027 | 0.000349171 | 22.61870748 | Hyper-methylated | PCa\|NT DMRs |
| chr14 | 101033362 | 101033979 | 2.04E−07 | 25.26836735 | Hyper-methylated | PCa\|NT DMRs |
| chr14 | 101106695 | 101107434 | 0.000215808 | 50.96560847 | Hyper-methylated | PCa\|NT DMRs |
| chr14 | 101131254 | 101132077 | 0.001969641 | −49.31349206 | Hypo-methylated | PCa\|NT DMRs |
| chr14 | 101172979 | 101173113 | 0.001111028 | 22.85449735 | Hyper-methylated | PCa\|NT DMRs |
| chr14 | 101174591 | 101175667 | 8.23E−07 | −34.67329932 | Hypo-methylated | PCa\|NT DMRs |
| chr14 | 101180323 | 101185461 | 0.009924186 | 20.81746032 | Hyper-methylated | PCa\|NT DMRs |
| chr14 | 101189225 | 101190570 | 2.00E−08 | −39.38095238 | Hypo-methylated | PCa\|NT DMRs |
| chr14 | 101263229 | 101284415 | 2.61E−13 | −27.41028139 | Hypo-methylated | PCa\|NT DMRs |
| chr14 | 101294315 | 101294508 | 0.003809353 | −21.52857143 | Hypo-methylated | PCa\|NT DMRs |
| chr14 | 101301380 | 101301859 | 4.10E−09 | −27.96904762 | Hypo-methylated | PCa\|NT DMRs |
| chr14 | 101302916 | 101311984 | 1.87E−08 | −29.14583333 | Hypo-methylated | PCa\|NT DMRs |
| chr14 | 101333924 | 101346783 | 1.13E−12 | −26.03462033 | Hypo-methylated | PCa\|NT DMRs |
| chr14 | 101518296 | 101528649 | 2.01E−07 | −30.74761905 | Hypo-methylated | PCa\|NT DMRs |
| chr14 | 101538880 | 101539975 | 4.69E−05 | −33.03630952 | Hypo-methylated | PCa\|NT DMRs |
| chr14 | 101578313 | 101578423 | 0.001969641 | 25.0952381 | Hyper-methylated | PCa\|NT DMRs |
| chr14 | 101579049 | 101585990 | 9.27E−10 | −26.77258687 | Hypo-methylated | PCa\|NT DMRs |
| chr14 | 101701119 | 101713820 | 3.68E−08 | −29.85205255 | Hypo-methylated | PCa\|NT DMRs |
| chr14 | 101826486 | 101836746 | 0.000243232 | −25.04696223 | Hypo-methylated | PCa\|NT DMRs |
| chr14 | 102101382 | 102156939 | 1.95E−12 | −21.61758242 | Hypo-methylated | PCa\|NT DMRs |
| chr14 | 102501541 | 102501768 | 0.001969641 | −41.46031746 | Hypo-methylated | PCa\|NT DMRs |
| chr14 | 102526995 | 102530115 | 0.000214774 | −36.67719298 | Hypo-methylated | PCa\|NT DMRs |
| chr14 | 102675913 | 102680519 | 0.000845293 | 29.67785714 | Hyper-methylated | PCa\|NT DMRs |
| chr14 | 102681469 | 102681712 | 1.97E−05 | −55.14857143 | Hypo-methylated | PCa\|NT DMRs |
| chr14 | 102964809 | 102964952 | 0.001423532 | −21.95238095 | Hypo-methylated | PCa\|NT DMRs |
| chr14 | 103389944 | 103390410 | 0.000175619 | 21.4021164 | Hyper-methylated | PCa\|NT DMRs |
| chr14 | 103394344 | 103395725 | 6.38E−27 | 22.46471088 | Hyper-methylated | PCa\|NT DMRs |
| chr14 | 103560935 | 103561069 | 0.003448944 | 29.23809524 | Hyper-methylated | PCa\|NT DMRs |
| chr14 | 103592992 | 103594052 | 1.96E−05 | −37.77962963 | Hypo-methylated | PCa\|NT DMRs |
| chr14 | 103603330 | 103603648 | 4.90E−06 | 39.20357143 | Hyper-methylated | PCa\|NT DMRs |
| chr14 | 103604943 | 103605422 | 8.71E−07 | 20.03581349 | Hyper-methylated | PCa\|NT DMRs |
| chr14 | 103745898 | 103747391 | 9.29E−07 | 24.15250965 | Hyper-methylated | PCa\|NT DMRs |
| chr14 | 103760337 | 103760870 | 0.005531225 | −32.47354497 | Hypo-methylated | PCa\|NT DMRs |
| chr14 | 104005066 | 104006787 | 0.000173442 | −23.19345238 | Hypo-methylated | PCa\|NT DMRs |
| chr14 | 104094770 | 104095033 | 0.001083007 | 26.81 | Hyper-methylated | PCa\|NT DMRs |
| chr14 | 104159363 | 104164598 | 0.002360289 | −31.95371429 | Hypo-methylated | PCa\|NT DMRs |
| chr14 | 104330466 | 104334123 | 0.001083007 | −44.30857143 | Hypo-methylated | PCa\|NT DMRs |
| chr14 | 104338087 | 104338789 | 1.66E−08 | 34.65026455 | Hyper-methylated | PCa\|NT DMRs |
| chr14 | 104339780 | 104345945 | 0.000632596 | −33.9327381 | Hypo-methylated | PCa\|NT DMRs |
| chr14 | 104561302 | 104562380 | 1.48E−05 | −27.57282913 | Hypo-methylated | PCa\|NT DMRs |
| chr14 | 104563219 | 104567800 | 2.11E−06 | −26.92216117 | Hypo-methylated | PCa\|NT DMRs |
| chr14 | 104596956 | 104600814 | 0.000234162 | −25.85659341 | Hypo-methylated | PCa\|NT DMRs |
| chr14 | 104609445 | 104610443 | 0.001129594 | −32.19206349 | Hypo-methylated | PCa\|NT DMRs |
| chr14 | 104622064 | 104622215 | 1.97E−05 | −35.89714286 | Hypo-methylated | PCa\|NT DMRs |
| chr14 | 104644569 | 104646477 | 1.99E−07 | −31.58864469 | Hypo-methylated | PCa\|NT DMRs |
| chr14 | 104689883 | 104690998 | 1.55E−05 | 25.92070393 | Hyper-methylated | PCa\|NT DMRs |
| chr14 | 104710740 | 104713530 | 2.06E−05 | −25.73469388 | Hypo-methylated | PCa\|NT DMRs |
| chr14 | 104741700 | 104749852 | 1.99E−07 | −32.47943723 | Hypo-methylated | PCa\|NT DMRs |
| chr14 | 104757434 | 104761709 | 7.32E−09 | −23.69530812 | Hypo-methylated | PCa\|NT DMRs |
| chr14 | 104799385 | 104799420 | 0.005968871 | 25.2952381 | Hyper-methylated | PCa\|NT DMRs |
| chr14 | 104799487 | 104801103 | 4.24E−07 | −21.35632184 | Hypo-methylated | PCa\|NT DMRs |
| chr14 | 104902476 | 104902897 | 5.67E−06 | 23.53571429 | Hyper-methylated | PCa\|NT DMRs |
| chr14 | 105054923 | 105055050 | 6.93E−06 | −65.31428571 | Hypo-methylated | PCa\|NT DMRs |
| chr14 | 105124904 | 105127312 | 0.000641983 | 26.11013825 | Hyper-methylated | PCa\|NT DMRs |
| chr14 | 105144478 | 105144982 | 0.00064571 | 34.25809524 | Hyper-methylated | PCa\|NT DMRs |
| chr14 | 105148179 | 105148235 | 0.008482405 | −30.84571429 | Hypo-methylated | PCa\|NT DMRs |
| chr14 | 105153420 | 105153951 | 0.003391914 | 33.93915344 | Hyper-methylated | PCa\|NT DMRs |
| chr14 | 105154445 | 105155328 | 9.85E−08 | −37.74497354 | Hypo-methylated | PCa\|NT DMRs |
| chr14 | 105166964 | 105167415 | 1.97E−05 | 30.88142857 | Hyper-methylated | PCa\|NT DMRs |
| chr14 | 105236410 | 105240179 | 0.000196948 | −20.77514881 | Hypo-methylated | PCa\|NT DMRs |
| chr14 | 105252117 | 105252339 | 0.000179253 | 20.18681319 | Hyper-methylated | PCa\|NT DMRs |
| chr14 | 105267701 | 105269175 | 0.001971273 | −26.14502165 | Hypo-methylated | PCa\|NT DMRs |
| chr14 | 105330015 | 105330536 | 0.00025637 | 27.56428571 | Hyper-methylated | PCa\|NT DMRs |
| chr14 | 105393144 | 105393233 | 0.000205174 | 31.17857143 | Hyper-methylated | PCa\|NT DMRs |
| chr14 | 105398331 | 105398969 | 4.00E−11 | −22.1237037 | Hypo-methylated | PCa\|NT DMRs |
| chr14 | 105399027 | 105399342 | 0.007716412 | −20.24671202 | Hypo-methylated | PCa\|NT DMRs |
| chr14 | 105435058 | 105444127 | 2.83E−05 | −22.03103175 | Hypo-methylated | PCa\|NT DMRs |
| chr14 | 105446449 | 105446720 | 1.42E−05 | −47.31802721 | Hypo-methylated | PCa\|NT DMRs |
| chr14 | 105525052 | 105528266 | 2.69E−13 | 40.76634383 | Hyper-methylated | PCa\|NT DMRs |
| chr14 | 105543326 | 105543602 | 0.004187875 | 34.03333333 | Hyper-methylated | PCa\|NT DMRs |
| chr14 | 105557746 | 105558069 | 1.97E−05 | −45.85095238 | Hypo-methylated | PCa\|NT DMRs |
| chr14 | 105633611 | 105633819 | 4.82E−06 | 24.76455026 | Hyper-methylated | PCa\|NT DMRs |
| chr14 | 105655398 | 105657755 | 0.001391421 | −23.21255411 | Hypo-methylated | PCa\|NT DMRs |
| chr14 | 105687906 | 105688130 | 0.000278967 | −59.5003663 | Hypo-methylated | PCa\|NT DMRs |
| chr14 | 105715867 | 105716237 | 0.000293608 | −28.92121212 | Hypo-methylated | PCa\|NT DMRs |
| chr14 | 105750529 | 105750614 | 0.00064571 | 49.71428571 | Hyper-methylated | PCa\|NT DMRs |
| chr14 | 105880799 | 105881035 | 0.004236974 | 35.22142857 | Hyper-methylated | PCa\|NT DMRs |
| chr14 | 105888433 | 105889659 | 8.30E−05 | 42.05026455 | Hyper-methylated | PCa\|NT DMRs |
| chr14 | 105935708 | 105936452 | 6.32E−05 | 26.29241071 | Hyper-methylated | PCa\|NT DMRs |
| chr14 | 105948819 | 105949229 | 0.005438982 | −21.40714286 | Hypo-methylated | PCa\|NT DMRs |
| chr14 | 106016106 | 106016165 | 1.97E−05 | −32.8947619 | Hypo-methylated | PCa\|NT DMRs |
| chr14 | 106344670 | 106348320 | 6.45E−05 | 34.8719888 | Hyper-methylated | PCa\|NT DMRs |
| chr14 | 106356880 | 106366615 | 6.88E−08 | 22.54772979 | Hyper-methylated | PCa\|NT DMRs |
| chr14 | 106734078 | 106743505 | 0.003314163 | −27.45714286 | Hypo-methylated | PCa\|NT DMRs |
| chr15 | 22029069 | 22042005 | 1.97E−05 | −27.96857143 | Hypo-methylated | PCa\|NT DMRs |
| chr15 | 22544822 | 22545409 | 3.19E−07 | −31.08333333 | Hypo-methylated | PCa\|NT DMRs |
| chr15 | 22825759 | 22828255 | 0.000189331 | −24.98484848 | Hypo-methylated | PCa\|NT DMRs |
| chr15 | 23032582 | 23032910 | 1.97E−05 | −32.14285714 | Hypo-methylated | PCa\|NT DMRs |
| chr15 | 23807225 | 23807592 | 2.41E−08 | 27.52070393 | Hyper-methylated | PCa\|NT DMRs |
| chr15 | 23888054 | 23891916 | 6.37E−06 | −29.03980952 | Hypo-methylated | PCa\|NT DMRs |
| chr15 | 24726006 | 24784826 | 3.17E−07 | −28.45897436 | Hypo-methylated | PCa\|NT DMRs |
| chr15 | 24943606 | 24992808 | 0.000339757 | −25.63982684 | Hypo-methylated | PCa\|NT DMRs |
| chr15 | 25143423 | 25200049 | 6.20E−09 | −20.78188153 | Hypo-methylated | PCa\|NT DMRs |
| chr15 | 26043821 | 26044589 | 0.006950763 | 30.3004329 | Hyper-methylated | PCa\|NT DMRs |
| chr15 | 26193752 | 26221074 | 5.94E−06 | −27.41774892 | Hypo-methylated | PCa\|NT DMRs |
| chr15 | 26263104 | 26302167 | 9.83E−09 | −28.70770308 | Hypo-methylated | PCa\|NT DMRs |
| chr15 | 26665873 | 26846396 | 0.001391421 | −27.20562771 | Hypo-methylated | PCa\|NT DMRs |
| chr15 | 27137410 | 27139864 | 1.92E−07 | 21.35371763 | Hyper-methylated | PCa\|NT DMRs |
| chr15 | 28070895 | 28070986 | 0.005968871 | −20.25714286 | Hypo-methylated | PCa\|NT DMRs |
| chr15 | 28753762 | 28753813 | 0.00097807 | 24.37312925 | Hyper-methylated | PCa\|NT DMRs |
| chr15 | 29130323 | 29130754 | 0.00064571 | 24.98148148 | Hyper-methylated | PCa\|NT DMRs |
| chr15 | 29423922 | 29424197 | 0.00068166 | 51.28869048 | Hyper-methylated | PCa\|NT DMRs |
| chr15 | 29572935 | 29573057 | 0.005968871 | 25.65714286 | Hyper-methylated | PCa\|NT DMRs |
| chr15 | 29967397 | 29968617 | 0.000286492 | 24.1484127 | Hyper-methylated | PCa\|NT DMRs |
| chr15 | 31476380 | 31499290 | 0.001604291 | −21.69285714 | Hypo-methylated | PCa\|NT DMRs |
| chr15 | 31508622 | 31516120 | 0.002894438 | 23.05993432 | Hyper-methylated | PCa\|NT DMRs |
| chr15 | 31603919 | 31604748 | 0.00641359 | −21.97619048 | Hypo-methylated | PCa\|NT DMRs |
| chr15 | 31630781 | 31658358 | 4.24E−07 | 22.9412415 | Hyper-methylated | PCa\|NT DMRs |
| chr15 | 31684723 | 31685698 | 0.00018587 | 35.68354978 | Hyper-methylated | PCa\|NT DMRs |
| chr15 | 34636940 | 34654458 | 1.78E−08 | −20.31873016 | Hypo-methylated | PCa\|NT DMRs |
| chr15 | 35014197 | 35014660 | 1.66E−07 | 41.70562771 | Hyper-methylated | PCa\|NT DMRs |
| chr15 | 35363399 | 35363504 | 0.001969641 | 34.07619048 | Hyper-methylated | PCa\|NT DMRs |
| chr15 | 37172485 | 37181088 | 2.66E−13 | 25.88128307 | Hyper-methylated | PCa\|NT DMRs |
| chr15 | 37697605 | 37874682 | 2.45E−07 | −26.89047619 | Hypo-methylated | PCa\|NT DMRs |
| chr15 | 37921054 | 38117040 | 0.00083549 | −26.3215873 | Hypo-methylated | PCa\|NT DMRs |
| chr15 | 38126318 | 38155343 | 0.005968871 | −33.45428571 | Hypo-methylated | PCa\|NT DMRs |
| chr15 | 39160002 | 39160211 | 0.00641359 | 28.16825397 | Hyper-methylated | PCa\|NT DMRs |
| chr15 | 39874437 | 39874777 | 0.000339627 | −21.46428571 | Hypo-methylated | PCa\|NT DMRs |
| chr15 | 40268678 | 40269241 | 0.000651745 | −44.16746032 | Hypo-methylated | PCa\|NT DMRs |
| chr15 | 40314882 | 40329305 | 0.004947537 | −22.45449735 | Hypo-methylated | PCa\|NT DMRs |
| chr15 | 40348308 | 40362086 | 2.28E−06 | −35.56289683 | Hypo-methylated | PCa\|NT DMRs |
| chr15 | 40395837 | 40396022 | 0.001969641 | −58.74047619 | Hypo-methylated | PCa\|NT DMRs |
| chr15 | 40576034 | 40576117 | 0.008234951 | 26.96666667 | Hyper-methylated | PCa\|NT DMRs |
| chr15 | 40627374 | 40627503 | 8.17E−05 | −32.33766234 | Hypo-methylated | PCa\|NT DMRs |
| chr15 | 40628591 | 40629344 | 0.007560755 | 27.81607143 | Hyper-methylated | PCa\|NT DMRs |
| chr15 | 40738481 | 40745036 | 0.001241245 | 22.26984127 | Hyper-methylated | PCa\|NT DMRs |
| chr15 | 41075917 | 41080617 | 0.007560755 | −32.58614719 | Hypo-methylated | PCa\|NT DMRs |
| chr15 | 41165191 | 41165916 | 0.006148156 | −23.32922824 | Hypo-methylated | PCa\|NT DMRs |
| chr15 | 41194603 | 41195487 | 4.39E−08 | 45.56604555 | Hyper-methylated | PCa\|NT DMRs |
| chr15 | 41217544 | 41218725 | 2.67E−16 | 28.69387755 | Hyper-methylated | PCa\|NT DMRs |
| chr15 | 41232703 | 41232933 | 0.005968871 | 34.34285714 | Hyper-methylated | PCa\|NT DMRs |
| chr15 | 41252097 | 41252982 | 0.004553481 | 25.41587302 | Hyper-methylated | PCa\|NT DMRs |
| chr15 | 42173883 | 42174854 | 5.95E−10 | 31.66821705 | Hyper-methylated | PCa\|NT DMRs |
| chr15 | 42185156 | 42185257 | 0.000205174 | 29.94940476 | Hyper-methylated | PCa\|NT DMRs |
| chr15 | 42447729 | 42470990 | 3.11E−05 | −32.25066667 | Hypo-methylated | PCa\|NT DMRs |
| chr15 | 42652080 | 42659502 | 2.85E−05 | 40.48717949 | Hyper-methylated | PCa\|NT DMRs |
| chr15 | 43579496 | 43580893 | 5.52E−05 | −33.95021645 | Hypo-methylated | PCa\|NT DMRs |
| chr15 | 44052919 | 44068997 | 3.62E−05 | −23.72539683 | Hypo-methylated | PCa\|NT DMRs |
| chr15 | 45467538 | 45479934 | 8.58E−13 | 30.99402428 | Hyper-methylated | PCa\|NT DMRs |
| chr15 | 45670456 | 45670952 | 2.74E−25 | 23.12908778 | Hyper-methylated | PCa\|NT DMRs |
| chr15 | 45942458 | 45942549 | 0.005968871 | 38.7 | Hyper-methylated | PCa\|NT DMRs |
| chr15 | 47292797 | 47392206 | 0.003448944 | −31.8414966 | Hypo-methylated | PCa\|NT DMRs |
| chr15 | 47477056 | 47477411 | 4.24E−06 | 29.00250627 | Hyper-methylated | PCa\|NT DMRs |
| chr15 | 52000774 | 52000949 | 0.001969641 | −62.75510204 | Hypo-methylated | PCa\|NT DMRs |
| chr15 | 52551211 | 52574767 | 0.003314163 | −23.25714286 | Hypo-methylated | PCa\|NT DMRs |
| chr15 | 53105843 | 53328080 | 1.79E−06 | −39.29952381 | Hypo-methylated | PCa\|NT DMRs |
| chr15 | 53591585 | 53802443 | 5.10E−08 | −33.66059113 | Hypo-methylated | PCa\|NT DMRs |
| chr15 | 55590327 | 55600864 | 0.009924186 | 22.24338624 | Hyper-methylated | PCa\|NT DMRs |
| chr15 | 55806775 | 55806837 | 0.005968871 | −30.99047619 | Hypo-methylated | PCa\|NT DMRs |
| chr15 | 59157047 | 59157300 | 2.80E−05 | 29.71111111 | Hyper-methylated | PCa\|NT DMRs |
| chr15 | 59730722 | 59730975 | 0.006589535 | 21.04047619 | Hyper-methylated | PCa\|NT DMRs |
| chr15 | 60084450 | 60084705 | 0.003622801 | −22.48809524 | Hypo-methylated | PCa\|NT DMRs |
| chr15 | 60237409 | 60285810 | 0.000109889 | −22.37983193 | Hypo-methylated | PCa\|NT DMRs |
| chr15 | 62042951 | 62043314 | 0.001969641 | −37.32857143 | Hypo-methylated | PCa\|NT DMRs |
| chr15 | 62411153 | 62437557 | 0.007428376 | −28.17802198 | Hypo-methylated | PCa\|NT DMRs |
| chr15 | 62456918 | 62457601 | 1.10E−09 | 21.52107143 | Hyper-methylated | PCa\|NT DMRs |
| chr15 | 62510408 | 62511293 | 0.000205174 | −29.41071429 | Hypo-methylated | PCa\|NT DMRs |
| chr15 | 62720479 | 62720729 | 0.008482405 | −59.83333333 | Hypo-methylated | PCa\|NT DMRs |
| chr15 | 63189297 | 63189537 | 3.44E−06 | −52.01785714 | Hypo-methylated | PCa\|NT DMRs |
| chr15 | 63192916 | 63192973 | 0.001969641 | 28.36734694 | Hyper-methylated | PCa\|NT DMRs |
| chr15 | 64447092 | 64453647 | 5.92E−05 | −22.78428571 | Hypo-methylated | PCa\|NT DMRs |
| chr15 | 64841547 | 64864948 | 0.001657405 | 23.48571429 | Hyper-methylated | PCa\|NT DMRs |
| chr15 | 65115885 | 65116069 | 0.005295761 | 24.59090909 | Hyper-methylated | PCa\|NT DMRs |
| chr15 | 65135286 | 65137216 | 0.001969641 | 45.19047619 | Hyper-methylated | PCa\|NT DMRs |
| chr15 | 65360510 | 65360538 | 0.002097193 | 21.28571429 | Hyper-methylated | PCa\|NT DMRs |
| chr15 | 65597874 | 65611914 | 2.78E−05 | −22.19627329 | Hypo-methylated | PCa\|NT DMRs |
| chr15 | 65707325 | 65708988 | 0.007560755 | −23.82142857 | Hypo-methylated | PCa\|NT DMRs |
| chr15 | 65715408 | 65715515 | 1.06E−06 | 21.35064935 | Hyper-methylated | PCa\|NT DMRs |
| chr15 | 66473053 | 66483605 | 0.001391421 | −26.30562771 | Hypo-methylated | PCa\|NT DMRs |
| chr15 | 66648693 | 66648786 | 0.004057568 | 25.61904762 | Hyper-methylated | PCa\|NT DMRs |
| chr15 | 66790377 | 66792977 | 0.005375157 | −21.93503401 | Hypo-methylated | PCa\|NT DMRs |
| chr15 | 66922915 | 66930993 | 0.000386041 | 31.30505952 | Hyper-methylated | PCa\|NT DMRs |
| chr15 | 66939822 | 66940000 | 0.001969641 | −30.8 | Hypo-methylated | PCa\|NT DMRs |
| chr15 | 66941645 | 66943046 | 0.001969641 | −53.49206349 | Hypo-methylated | PCa\|NT DMRs |
| chr15 | 66965864 | 66965923 | 0.005968871 | 47.93809524 | Hyper-methylated | PCa\|NT DMRs |
| chr15 | 67413302 | 67413801 | 1.36E−05 | 44.53741497 | Hyper-methylated | PCa\|NT DMRs |
| chr15 | 68116472 | 68116935 | 2.80E−05 | 22.32063492 | Hyper-methylated | PCa\|NT DMRs |
| chr15 | 68498070 | 68502604 | 0.001391421 | 25.22789116 | Hyper-methylated | PCa\|NT DMRs |
| chr15 | 70354718 | 70354835 | 6.94E−06 | −47.44761905 | Hypo-methylated | PCa\|NT DMRs |
| chr15 | 70641449 | 70641603 | 0.00064571 | 51.0122449 | Hyper-methylated | PCa\|NT DMRs |
| chr15 | 70740904 | 70744856 | 0.004794367 | 29.18367347 | Hyper-methylated | PCa\|NT DMRs |
| chr15 | 71507012 | 71529969 | 0.001047828 | 22.50576441 | Hyper-methylated | PCa\|NT DMRs |
| chr15 | 72472500 | 72472793 | 0.004149771 | −37.76923077 | Hypo-methylated | PCa\|NT DMRs |
| chr15 | 72519739 | 72520689 | 0.001969641 | 36.27063492 | Hyper-methylated | PCa\|NT DMRs |
| chr15 | 72668088 | 72669048 | 5.76E−11 | 41.44086022 | Hyper-methylated | PCa\|NT DMRs |
| chr15 | 72979666 | 72979871 | 0.000974795 | −28.02678571 | Hypo-methylated | PCa\|NT DMRs |
| chr15 | 73611223 | 73611529 | 3.39E−07 | 25.79911817 | Hyper-methylated | PCa\|NT DMRs |
| chr15 | 73617793 | 73617969 | 6.42E−05 | 24.41269841 | Hyper-methylated | PCa\|NT DMRs |
| chr15 | 74215872 | 74219208 | 1.19E−05 | 26.03219048 | Hyper-methylated | PCa\|NT DMRs |
| chr15 | 74234285 | 74244344 | 0.002082381 | 27.4789916 | Hyper-methylated | PCa\|NT DMRs |
| chr15 | 74249177 | 74249231 | 0.000974795 | 22.94642857 | Hyper-methylated | PCa\|NT DMRs |
| chr15 | 74400269 | 74419179 | 0.000242379 | −28.42666667 | Hypo-methylated | PCa\|NT DMRs |
| chr15 | 74484446 | 74484870 | 0.000392715 | −24.98095238 | Hypo-methylated | PCa\|NT DMRs |
| chr15 | 74564053 | 74591576 | 6.02E−06 | −26.46252588 | Hypo-methylated | PCa\|NT DMRs |
| chr15 | 74658079 | 74658466 | 3.09E−12 | 29.84555985 | Hyper-methylated | PCa\|NT DMRs |
| chr15 | 75018814 | 75019336 | 0.00355142 | 27.76007326 | Hyper-methylated | PCa\|NT DMRs |
| chr15 | 75069591 | 75069761 | 0.000205174 | 56.06845238 | Hyper-methylated | PCa\|NT DMRs |
| chr15 | 75213040 | 75213089 | 0.005968871 | 35.85714286 | Hyper-methylated | PCa\|NT DMRs |
| chr15 | 75251061 | 75254698 | 2.48E−08 | 31.83384813 | Hyper-methylated | PCa\|NT DMRs |
| chr15 | 75470918 | 75471350 | 2.24E−14 | 38.82336656 | Hyper-methylated | PCa\|NT DMRs |
| chr15 | 75614969 | 75620496 | 6.69E−05 | −20.72261905 | Hypo-methylated | PCa\|NT DMRs |
| chr15 | 75940744 | 75941919 | 0.001795658 | −20.96645022 | Hypo-methylated | PCa\|NT DMRs |
| chr15 | 75969571 | 75974521 | 0.001836557 | −23.29304029 | Hypo-methylated | PCa\|NT DMRs |
| chr15 | 76257288 | 76257491 | 0.00064571 | −66.26530612 | Hypo-methylated | PCa\|NT DMRs |
| chr15 | 76484051 | 76487308 | 9.19E−10 | 32.96597403 | Hyper-methylated | PCa\|NT DMRs |
| chr15 | 77271846 | 77271970 | 0.005968871 | −24.34761905 | Hypo-methylated | PCa\|NT DMRs |
| chr15 | 77861900 | 77862131 | 0.000176046 | 25.7012987 | Hyper-methylated | PCa\|NT DMRs |
| chr15 | 77874305 | 77874698 | 0.003032307 | −24.3792517 | Hypo-methylated | PCa\|NT DMRs |
| chr15 | 77949978 | 77952727 | 0.001899053 | −24.6302521 | Hypo-methylated | PCa\|NT DMRs |
| chr15 | 78090566 | 78090777 | 0.00068166 | −35.64464286 | Hypo-methylated | PCa\|NT DMRs |
| chr15 | 78108412 | 78111252 | 1.32E−05 | 24.08378926 | Hyper-methylated | PCa\|NT DMRs |
| chr15 | 78163297 | 78163822 | 0.004095673 | −35.88095238 | Hypo-methylated | PCa\|NT DMRs |
| chr15 | 78235966 | 78237083 | 1.13E−08 | 23.22563025 | Hyper-methylated | PCa\|NT DMRs |
| chr15 | 78911719 | 78912591 | 7.68E−06 | 23.06105006 | Hyper-methylated | PCa\|NT DMRs |
| chr15 | 79122995 | 79123539 | 1.92E−11 | 37.98126294 | Hyper-methylated | PCa\|NT DMRs |
| chr15 | 79471392 | 79497517 | 7.44E−05 | −30.37936508 | Hypo-methylated | PCa\|NT DMRs |
| chr15 | 79502068 | 79502228 | 0.000205174 | 37.28452381 | Hyper-methylated | PCa\|NT DMRs |
| chr15 | 79725328 | 79743977 | 0.009069241 | 26.00779221 | Hyper-methylated | PCa\|NT DMRs |
| chr15 | 82247468 | 82306639 | 0.007560755 | −20.01071429 | Hypo-methylated | PCa\|NT DMRs |
| chr15 | 83317801 | 83331629 | 0.001969641 | −26.45442177 | Hypo-methylated | PCa\|NT DMRs |
| chr15 | 84748595 | 84748733 | 6.99E−08 | 20.45341615 | Hyper-methylated | PCa\|NT DMRs |
| chr15 | 84923577 | 84946482 | 0.005968871 | −27.78095238 | Hypo-methylated | PCa\|NT DMRs |
| chr15 | 85145308 | 85145478 | 0.000205174 | −32.28571429 | Hypo-methylated | PCa\|NT DMRs |
| chr15 | 85398294 | 85398498 | 0.005968871 | −61.98095238 | Hypo-methylated | PCa\|NT DMRs |
| chr15 | 87182868 | 87415020 | 0.009084137 | −20.87179487 | Hypo-methylated | PCa\|NT DMRs |
| chr15 | 87522880 | 88296924 | 1.63E−17 | −29.88275862 | Hypo-methylated | PCa\|NT DMRs |
| chr15 | 88447542 | 88522657 | 1.60E−13 | −25.25228175 | Hypo-methylated | PCa\|NT DMRs |
| chr15 | 88596574 | 88621644 | 1.16E−05 | −22.45192744 | Hypo-methylated | PCa\|NT DMRs |
| chr15 | 88649335 | 88749293 | 5.23E−09 | −30.45780731 | Hypo-methylated | PCa\|NT DMRs |
| chr15 | 88842943 | 88939251 | 7.48E−08 | −31.36912442 | Hypo-methylated | PCa\|NT DMRs |
| chr15 | 89174997 | 89175108 | 0.004095673 | −67.37619048 | Hypo-methylated | PCa\|NT DMRs |
| chr15 | 89182658 | 89182811 | 0.00064571 | −25.92857143 | Hypo-methylated | PCa\|NT DMRs |
| chr15 | 89200431 | 89246883 | 3.02E−07 | −30.17417582 | Hypo-methylated | PCa\|NT DMRs |
| chr15 | 89254550 | 89273482 | 0.001241245 | −27.47883598 | Hypo-methylated | PCa\|NT DMRs |
| chr15 | 89438128 | 89442224 | 4.04E−12 | 23.12242063 | Hyper-methylated | PCa\|NT DMRs |
| chr15 | 89455546 | 89455938 | 0.000122432 | 41.36580087 | Hyper-methylated | PCa\|NT DMRs |
| chr15 | 89682168 | 89691275 | 0.002568162 | −20.08253968 | Hypo-methylated | PCa\|NT DMRs |
| chr15 | 89903447 | 89903705 | 3.63E−06 | 30.87328042 | Hyper-methylated | PCa\|NT DMRs |
| chr15 | 89905949 | 89906200 | 5.36E−06 | 22.40761905 | Hyper-methylated | PCa\|NT DMRs |
| chr15 | 90320205 | 90320635 | 9.08E−07 | 28.35497835 | Hyper-methylated | PCa\|NT DMRs |
| chr15 | 90345456 | 90347438 | 0.00064571 | 31.29251701 | Hyper-methylated | PCa\|NT DMRs |
| chr15 | 90608490 | 90609864 | 0.005685126 | 30.23992674 | Hyper-methylated | PCa\|NT DMRs |
| chr15 | 90693374 | 90703780 | 0.000294557 | −33.5952381 | Hypo-methylated | PCa\|NT DMRs |
| chr15 | 90717795 | 90717995 | 0.00023371 | −55.99047619 | Hypo-methylated | PCa\|NT DMRs |
| chr15 | 90727205 | 90728045 | 1.54E−05 | −27.51316527 | Hypo-methylated | PCa\|NT DMRs |
| chr15 | 90754353 | 90755501 | 0.006410012 | 21.93290043 | Hyper-methylated | PCa\|NT DMRs |
| chr15 | 90768876 | 90771804 | 0.00025637 | −21.3234127 | Hypo-methylated | PCa\|NT DMRs |
| chr15 | 91413479 | 91414465 | 0.005513661 | 20.7952381 | Hyper-methylated | PCa\|NT DMRs |
| chr15 | 91438912 | 91439061 | 0.001115302 | −30.39285714 | Hypo-methylated | PCa\|NT DMRs |
| chr15 | 91822078 | 92043600 | 6.55E−07 | −25.92301587 | Hypo-methylated | PCa\|NT DMRs |
| chr15 | 92172082 | 92194203 | 0.00064571 | −44.41904762 | Hypo-methylated | PCa\|NT DMRs |
| chr15 | 92231226 | 92383271 | 8.32E−09 | −35.70816327 | Hypo-methylated | PCa\|NT DMRs |
| chr15 | 92884266 | 92930804 | 1.40E−05 | −31.58928571 | Hypo-methylated | PCa\|NT DMRs |
| chr15 | 93315575 | 93316290 | 0.002097193 | −40.53061224 | Hypo-methylated | PCa\|NT DMRs |
| chr15 | 93355684 | 93355890 | 0.001969641 | −45.78571429 | Hypo-methylated | PCa\|NT DMRs |
| chr15 | 93590747 | 93592053 | 0.003032307 | 30.83650794 | Hyper-methylated | PCa\|NT DMRs |
| chr15 | 93664314 | 93687756 | 2.32E−06 | −41.432493 | Hypo-methylated | PCa\|NT DMRs |
| chr15 | 93700711 | 93715169 | 4.05E−06 | −35.07857143 | Hypo-methylated | PCa\|NT DMRs |
| chr15 | 93881119 | 93897807 | 1.42E−05 | −35.0452381 | Hypo-methylated | PCa\|NT DMRs |
| chr15 | 93958496 | 94006028 | 1.45E−08 | −25.09396825 | Hypo-methylated | PCa\|NT DMRs |
| chr15 | 94072338 | 94518572 | 4.61E−18 | −29.1688254 | Hypo-methylated | PCa\|NT DMRs |
| chr15 | 96170203 | 96170334 | 0.005968871 | 48.18761905 | Hyper-methylated | PCa\|NT DMRs |
| chr15 | 96828892 | 96865061 | 0.002280453 | 22.24302135 | Hyper-methylated | PCa\|NT DMRs |
| chr15 | 96865958 | 96866815 | 4.34E−11 | 30.44519141 | Hyper-methylated | PCa\|NT DMRs |
| chr15 | 96884578 | 96899722 | 1.49E−13 | 25.76121032 | Hyper-methylated | PCa\|NT DMRs |
| chr15 | 97977700 | 97992575 | 0.00064571 | −36.29931973 | Hypo-methylated | PCa\|NT DMRs |
| chr15 | 98836205 | 98836262 | 4.10E−07 | 21.92481203 | Hyper-methylated | PCa\|NT DMRs |
| chr15 | 99091076 | 99091263 | 0.004095673 | 42.92857143 | Hyper-methylated | PCa\|NT DMRs |
| chr15 | 99289458 | 99289756 | 0.005968871 | −32.44571429 | Hypo-methylated | PCa\|NT DMRs |
| chr15 | 99488657 | 99489989 | 0.001083007 | 28.66190476 | Hyper-methylated | PCa\|NT DMRs |
| chr15 | 99494842 | 99497465 | 0.003510453 | −21.43877551 | Hypo-methylated | PCa\|NT DMRs |
| chr15 | 99968347 | 99968608 | 0.000205174 | −37.87202381 | Hypo-methylated | PCa\|NT DMRs |
| chr15 | 100289436 | 100295420 | 0.009807302 | −39.44238095 | Hypo-methylated | PCa\|NT DMRs |
| chr15 | 101306522 | 101306688 | 0.001969641 | 24.75238095 | Hyper-methylated | PCa\|NT DMRs |
| chr15 | 101434708 | 101442803 | 0.00358897 | −37.5415873 | Hypo-methylated | PCa\|NT DMRs |
| chr15 | 101703020 | 101703297 | 0.001969641 | −31.13492063 | Hypo-methylated | PCa\|NT DMRs |
| chr15 | 101715537 | 101715798 | 0.000205174 | 50.61607143 | Hyper-methylated | PCa\|NT DMRs |
| chr15 | 101729380 | 101729709 | 0.000481535 | −47.57777778 | Hypo-methylated | PCa\|NT DMRs |
| chr15 | 101807224 | 101816797 | 0.002313796 | −24.37678571 | Hypo-methylated | PCa\|NT DMRs |
| chr15 | 101938428 | 101938823 | 1.76E−06 | −52.53174603 | Hypo-methylated | PCa\|NT DMRs |
| chr16 | 129587 | 129718 | 0.000205174 | 24.92857143 | Hyper-methylated | PCa\|NT DMRs |
| chr16 | 316409 | 318893 | 1.02E−05 | −37.50331263 | Hypo-methylated | PCa\|NT DMRs |
| chr16 | 374770 | 375243 | 9.58E−07 | 52.56491228 | Hyper-methylated | PCa\|NT DMRs |
| chr16 | 377411 | 377654 | 0.001181579 | −21.68027211 | Hypo-methylated | PCa\|NT DMRs |
| chr16 | 390151 | 390199 | 0.005968871 | 42.69142857 | Hyper-methylated | PCa\|NT DMRs |
| chr16 | 419880 | 420151 | 0.003314163 | −33.37428571 | Hypo-methylated | PCa\|NT DMRs |
| chr16 | 452296 | 452626 | 0.006589535 | −38.58571429 | Hypo-methylated | PCa\|NT DMRs |
| chr16 | 507998 | 508061 | 0.004095673 | 21.70634921 | Hyper-methylated | PCa\|NT DMRs |
| chr16 | 521358 | 521474 | 0.001969641 | 44.68650794 | Hyper-methylated | PCa\|NT DMRs |
| chr16 | 579442 | 584276 | 4.08E−09 | −24.02521008 | Hypo-methylated | PCa\|NT DMRs |
| chr16 | 596832 | 597005 | 2.31E−05 | 29.81758242 | Hyper-methylated | PCa\|NT DMRs |
| chr16 | 607930 | 608330 | 0.00179283 | 44.71130952 | Hyper-methylated | PCa\|NT DMRs |
| chr16 | 640733 | 640960 | 0.005513661 | −28.38154762 | Hypo-methylated | PCa\|NT DMRs |
| chr16 | 648872 | 651537 | 5.76E−05 | −31.77063492 | Hypo-methylated | PCa\|NT DMRs |
| chr16 | 666321 | 667486 | 0.000205174 | −45.48214286 | Hypo-methylated | PCa\|NT DMRs |
| chr16 | 695119 | 698338 | 1.21E−06 | −23.22914863 | Hypo-methylated | PCa\|NT DMRs |
| chr16 | 731089 | 733945 | 6.61E−06 | −22.893912 | Hypo-methylated | PCa\|NT DMRs |
| chr16 | 771702 | 772690 | 7.76E−07 | 20.40168971 | Hyper-methylated | PCa\|NT DMRs |
| chr16 | 800340 | 801597 | 8.84E−07 | −41.60869565 | Hypo-methylated | PCa\|NT DMRs |
| chr16 | 804931 | 805156 | 0.005542011 | 26.03463203 | Hyper-methylated | PCa\|NT DMRs |
| chr16 | 809405 | 809722 | 4.62E−05 | −35.01904762 | Hypo-methylated | PCa\|NT DMRs |
| chr16 | 824130 | 824399 | 0.000118733 | −39.66031746 | Hypo-methylated | PCa\|NT DMRs |
| chr16 | 831290 | 831600 | 0.000822388 | 30.80519481 | Hyper-methylated | PCa\|NT DMRs |
| chr16 | 833232 | 834438 | 0.00016113 | −37.3397619 | Hypo-methylated | PCa\|NT DMRs |
| chr16 | 845365 | 846078 | 1.82E−06 | −44.38644689 | Hypo-methylated | PCa\|NT DMRs |
| chr16 | 879975 | 880371 | 0.007433023 | 25.80753968 | Hyper-methylated | PCa\|NT DMRs |
| chr16 | 881016 | 881348 | 1.82E−06 | 43.82783883 | Hyper-methylated | PCa\|NT DMRs |
| chr16 | 881738 | 883205 | 0.004173168 | −24.24126984 | Hypo-methylated | PCa\|NT DMRs |
| chr16 | 1017270 | 1017506 | 6.69E−05 | −23.56785714 | Hypo-methylated | PCa\|NT DMRs |
| chr16 | 1027225 | 1027604 | 0.001241245 | −30.63186813 | Hypo-methylated | PCa\|NT DMRs |
| chr16 | 1032361 | 1032730 | 3.60E−06 | 21.51498599 | Hyper-methylated | PCa\|NT DMRs |
| chr16 | 1054132 | 1054494 | 0.00064571 | 52.65646259 | Hyper-methylated | PCa\|NT DMRs |
| chr16 | 1116428 | 1119025 | 4.23E−06 | −26.82653061 | Hypo-methylated | PCa\|NT DMRs |
| chr16 | 1128026 | 1129023 | 1.42E−10 | −41.94964286 | Hypo-methylated | PCa\|NT DMRs |
| chr16 | 1138649 | 1140099 | 0.001975042 | −28.11002506 | Hypo-methylated | PCa\|NT DMRs |
| chr16 | 1143516 | 1144910 | 9.64E−13 | 30.60883257 | Hyper-methylated | PCa\|NT DMRs |
| chr16 | 1147838 | 1148922 | 0.002185514 | −25.15674603 | Hypo-methylated | PCa\|NT DMRs |
| chr16 | 1150350 | 1152475 | 1.25E−05 | −25.52354497 | Hypo-methylated | PCa\|NT DMRs |
| chr16 | 1167634 | 1168050 | 0.00064571 | −28.22789116 | Hypo-methylated | PCa\|NT DMRs |
| chr16 | 1209811 | 1210944 | 6.15E−17 | −40.77067271 | Hypo-methylated | PCa\|NT DMRs |
| chr16 | 1213453 | 1213857 | 0.000491935 | 26.35 | Hyper-methylated | PCa\|NT DMRs |
| chr16 | 1238767 | 1238824 | 0.001969641 | 29.04761905 | Hyper-methylated | PCa\|NT DMRs |
| chr16 | 1294104 | 1295456 | 5.68E−08 | 28.52904762 | Hyper-methylated | PCa\|NT DMRs |
| chr16 | 1296216 | 1297328 | 0.001789867 | −31.01904762 | Hypo-methylated | PCa\|NT DMRs |
| chr16 | 1312250 | 1312413 | 0.001850915 | −22.75 | Hypo-methylated | PCa\|NT DMRs |
| chr16 | 1332687 | 1333610 | 9.81E−06 | 32.34391534 | Hyper-methylated | PCa\|NT DMRs |
| chr16 | 1338204 | 1338392 | 0.006410012 | −31.67857143 | Hypo-methylated | PCa\|NT DMRs |
| chr16 | 1339750 | 1340288 | 0.000215808 | 39.78941799 | Hyper-methylated | PCa\|NT DMRs |
| chr16 | 1368728 | 1374052 | 0.000261046 | −23.57653061 | Hypo-methylated | PCa\|NT DMRs |
| chr16 | 1382482 | 1383057 | 4.56E−08 | 31.74450549 | Hyper-methylated | PCa\|NT DMRs |
| chr16 | 1400730 | 1401063 | 0.000494377 | −20.5085034 | Hypo-methylated | PCa\|NT DMRs |
| chr16 | 1416974 | 1418827 | 1.59E−06 | −42.14744898 | Hypo-methylated | PCa\|NT DMRs |
| chr16 | 1477905 | 1478746 | 0.000339079 | 29.43596059 | Hyper-methylated | PCa\|NT DMRs |
| chr16 | 1480657 | 1481065 | 1.16E−05 | −24.0244898 | Hypo-methylated | PCa\|NT DMRs |
| chr16 | 1510707 | 1510800 | 0.001139334 | −26.43650794 | Hypo-methylated | PCa\|NT DMRs |
| chr16 | 1544069 | 1545020 | 0.0009659 | −27.6046176 | Hypo-methylated | PCa\|NT DMRs |
| chr16 | 1606297 | 1606481 | 0.001969641 | −46.36507937 | Hypo-methylated | PCa\|NT DMRs |
| chr16 | 1729914 | 1730328 | 2.85E−05 | −55.95604396 | Hypo-methylated | PCa\|NT DMRs |
| chr16 | 1737392 | 1748877 | 2.28E−05 | −29.48150183 | Hypo-methylated | PCa\|NT DMRs |
| chr16 | 1762445 | 1763089 | 0.000331541 | 20.81255411 | Hyper-methylated | PCa\|NT DMRs |
| chr16 | 1829686 | 1831723 | 1.72E−05 | −28.79231951 | Hypo-methylated | PCa\|NT DMRs |
| chr16 | 1843515 | 1844184 | 7.47E−06 | −32.26031746 | Hypo-methylated | PCa\|NT DMRs |
| chr16 | 1963218 | 1963688 | 0.009503366 | −29.73214286 | Hypo-methylated | PCa\|NT DMRs |
| chr16 | 1988972 | 1989208 | 0.001969641 | 30.27539683 | Hyper-methylated | PCa\|NT DMRs |
| chr16 | 2013337 | 2013474 | 0.005968871 | −33.3247619 | Hypo-methylated | PCa\|NT DMRs |
| chr16 | 2040902 | 2041613 | 6.80E−09 | 27.18174603 | Hyper-methylated | PCa\|NT DMRs |
| chr16 | 2042480 | 2042875 | 1.74E−08 | 25.47312169 | Hyper-methylated | PCa\|NT DMRs |
| chr16 | 2077489 | 2079711 | 0.000457787 | −22.55531136 | Hypo-methylated | PCa\|NT DMRs |
| chr16 | 2082985 | 2083178 | 0.007560755 | 22.00416667 | Hyper-methylated | PCa\|NT DMRs |
| chr16 | 2083393 | 2083684 | 6.42E−05 | 21.6031746 | Hyper-methylated | PCa\|NT DMRs |
| chr16 | 2094109 | 2094645 | 0.004095673 | 45.76984127 | Hyper-methylated | PCa\|NT DMRs |
| chr16 | 2139882 | 2141984 | 2.25E−12 | −33.41763192 | Hypo-methylated | PCa\|NT DMRs |
| chr16 | 2173124 | 2176198 | 2.74E−08 | 26.98794326 | Hyper-methylated | PCa\|NT DMRs |
| chr16 | 2212733 | 2213016 | 1.42E−05 | −25.8707483 | Hypo-methylated | PCa\|NT DMRs |
| chr16 | 2261316 | 2264037 | 0.001337734 | −24.2162465 | Hypo-methylated | PCa\|NT DMRs |
| chr16 | 2286538 | 2288301 | 1.97E−17 | 26.84145658 | Hyper-methylated | PCa\|NT DMRs |
| chr16 | 2310317 | 2315610 | 0.000836132 | −44.47619048 | Hypo-methylated | PCa\|NT DMRs |
| chr16 | 2319266 | 2319666 | 0.009807302 | −24.76904762 | Hypo-methylated | PCa\|NT DMRs |
| chr16 | 2323514 | 2323894 | 8.96E−05 | −48.93253968 | Hypo-methylated | PCa\|NT DMRs |
| chr16 | 2391323 | 2392106 | 9.81E−13 | 26.07663004 | Hyper-methylated | PCa\|NT DMRs |
| chr16 | 2531115 | 2531267 | 0.001969641 | −43.38650794 | Hypo-methylated | PCa\|NT DMRs |
| chr16 | 2536085 | 2537560 | 6.69E−05 | 38.79047619 | Hyper-methylated | PCa\|NT DMRs |
| chr16 | 2556878 | 2557498 | 0.006589535 | 38.23285714 | Hyper-methylated | PCa\|NT DMRs |
| chr16 | 2559903 | 2563541 | 0.00641359 | 24.86507937 | Hyper-methylated | PCa\|NT DMRs |
| chr16 | 2569458 | 2569997 | 0.003162339 | −33.55238095 | Hypo-methylated | PCa\|NT DMRs |
| chr16 | 2579575 | 2579747 | 0.000339757 | −24.28571429 | Hypo-methylated | PCa\|NT DMRs |
| chr16 | 2587001 | 2587382 | 0.001789867 | −32.88253968 | Hypo-methylated | PCa\|NT DMRs |
| chr16 | 2731606 | 2731884 | 0.003583021 | −28.56428571 | Hypo-methylated | PCa\|NT DMRs |
| chr16 | 2740098 | 2740473 | 0.001466402 | 41.04285714 | Hyper-methylated | PCa\|NT DMRs |
| chr16 | 2753295 | 2753532 | 6.42E−05 | −52.0952381 | Hypo-methylated | PCa\|NT DMRs |
| chr16 | 2767656 | 2768022 | 0.002585277 | 30.85119048 | Hyper-methylated | PCa\|NT DMRs |
| chr16 | 2770784 | 2770842 | 0.007573168 | 22.00340136 | Hyper-methylated | PCa\|NT DMRs |
| chr16 | 2774824 | 2775168 | 0.007560755 | −22.30952381 | Hypo-methylated | PCa\|NT DMRs |
| chr16 | 3002543 | 3003018 | 0.002437742 | −41.31216931 | Hypo-methylated | PCa\|NT DMRs |
| chrl6 | 3008659 | 3010399 | 0.005578507 | −35.06227106 | Hypo-methylated | PCa\|NT DMRs |
| chr16 | 3096651 | 3105181 | 1.92E−08 | 26.14668737 | Hyper-methylated | PCa\|NT DMRs |
| chr16 | 3142132 | 3143251 | 1.24E−05 | −35.07563025 | Hypo-methylated | PCa\|NT DMRs |
| chr16 | 3221699 | 3222366 | 5.01E−08 | 21.03040293 | Hyper-methylated | PCa\|NT DMRs |
| chr16 | 3230711 | 3230853 | 0.002373155 | 20.19047619 | Hyper-methylated | PCa\|NT DMRs |
| chr16 | 3237212 | 3238613 | 8.38E−05 | 20.60451128 | Hyper-methylated | PCa\|NT DMRs |
| chr16 | 3245574 | 3257521 | 0.00179283 | −30.85059524 | Hypo-methylated | PCa\|NT DMRs |
| chr16 | 3345159 | 3345415 | 0.000122527 | 21.06971429 | Hyper-methylated | PCa\|NT DMRs |
| chr16 | 3400658 | 3406974 | 5.20E−08 | −21.15363409 | Hypo-methylated | PCa\|NT DMRs |
| chr16 | 3550066 | 3550247 | 4.16E−07 | 34.70327381 | Hyper-methylated | PCa\|NT DMRs |
| chr16 | 3625561 | 3630897 | 7.45E−05 | 29.80431548 | Hyper-methylated | PCa\|NT DMRs |
| chr16 | 3639022 | 3641715 | 1.60E−07 | 28.85714286 | Hyper-methylated | PCa\|NT DMRs |
| chr16 | 3695592 | 3696467 | 0.000783791 | −37.92857143 | Hypo-methylated | PCa\|NT DMRs |
| chr16 | 3746410 | 3746889 | 0.00064571 | −52.8877551 | Hypo-methylated | PCa\|NT DMRs |
| chr16 | 3770441 | 3774701 | 0.001171318 | −20.73917749 | Hypo-methylated | PCa\|NT DMRs |
| chr16 | 3999452 | 4004641 | 4.41E−05 | 34.07006803 | Hyper-methylated | PCa\|NT DMRs |
| chr16 | 4008010 | 4008280 | 0.006743251 | −27.22433862 | Hypo-methylated | PCa\|NT DMRs |
| chr16 | 4366670 | 4368899 | 0.001022616 | −24.48138528 | Hypo-methylated | PCa\|NT DMRs |
| chr16 | 4452456 | 4453668 | 0.006518597 | −30.84081633 | Hypo-methylated | PCa\|NT DMRs |
| chr16 | 4464451 | 4465763 | 0.009106413 | 45.51269841 | Hyper-methylated | PCa\|NT DMRs |
| chr16 | 4587619 | 4588433 | 3.15E−11 | 24.92796353 | Hyper-methylated | PCa\|NT DMRs |
| chr16 | 4686197 | 4687202 | 0.000200126 | 30.66849817 | Hyper-methylated | PCa\|NT DMRs |
| chr16 | 4710700 | 4717443 | 3.61E−06 | 32.68673469 | Hyper-methylated | PCa\|NT DMRs |
| chr16 | 4723493 | 4723653 | 0.000190021 | −25.71428571 | Hypo-methylated | PCa\|NT DMRs |
| chr16 | 4739859 | 4741376 | 0.000103014 | 25.95658263 | Hyper-methylated | PCa\|NT DMRs |
| chr16 | 4851034 | 4851345 | 0.003464153 | 20.3333333 | Hyper-methylated | PCa\|NT DMRs |
| chr16 | 4854036 | 4864547 | 0.000210841 | −28.4952381 | Hypo-methylated | PCa\|NT DMRs |
| chr16 | 5030424 | 5038314 | 2.93E−05 | 30.95714286 | Hyper-methylated | PCa\|NT DMRs |
| chr16 | 5298577 | 5537115 | 3.20E−13 | −34.68508403 | Hypo-methylated | PCa\|NT DMRs |
| chr16 | 5571821 | 6049568 | 2.15E−29 | −35.58996728 | Hypo-methylated | PCa\|NT DMRs |
| chr16 | 6049600 | 6056311 | 0.004553481 | −32.63544974 | Hypo-methylated | PCa\|NT DMRs |
| chr16 | 6093876 | 6523101 | 4.85E−27 | −34.88836996 | Hypo-methylated | PCa\|NT DMRs |
| chr16 | 6695394 | 6771171 | 1.59E−11 | −31.27422969 | Hypo-methylated | PCa\|NT DMRs |
| chr16 | 6865061 | 7338727 | 3.17E−31 | −34.86954436 | Hypo-methylated | PCa\|NT DMRs |
| chr16 | 7359520 | 7694298 | 2.47E−17 | −37.27677656 | Hypo-methylated | PCa\|NT DMRs |
| chr16 | 7710748 | 8148995 | 1.28E−14 | −38.47738095 | Hypo-methylated | PCa\|NT DMRs |
| chr16 | 8158434 | 8609206 | 1.19E−31 | −35.50872314 | Hypo-methylated | PCa\|NT DMRs |
| chr16 | 8779964 | 8780063 | 0.005968871 | −53.99428571 | Hypo-methylated | PCa\|NT DMRs |
| chr16 | 8808710 | 8815433 | 0.000118733 | −39.26455026 | Hypo-methylated | PCa\|NT DMRs |
| chr16 | 8960174 | 8960904 | 4.11E−07 | −54.43007519 | Hypo-methylated | PCa\|NT DMRs |
| chr16 | 8963552 | 8963808 | 1.34E−05 | 31.58843537 | Hyper-methylated | PCa\|NT DMRs |
| chr16 | 9107009 | 9107319 | 2.81E−09 | 42.40384615 | Hyper-methylated | PCa\|NT DMRs |
| chr16 | 9184207 | 9184407 | 8.69E−05 | 22.28163265 | Hyper-methylated | PCa\|NT DMRs |
| chr16 | 9248446 | 9250894 | 0.003034084 | 22.55782313 | Hyper-methylated | PCa\|NT DMRs |
| chr16 | 9283984 | 9335815 | 9.05E−09 | −34.52765568 | Hypo-methylated | PCa\|NT DMRs |
| chr16 | 10481704 | 10482044 | 0.000117699 | −34.77922078 | Hypo-methylated | PCa\|NT DMRs |
| chr16 | 10652798 | 10653288 | 0.000974795 | −63.29761905 | Hypo-methylated | PCa\|NT DMRs |
| chr16 | 10716275 | 10721971 | 0.00641359 | −21.97619048 | Hypo-methylated | PCa\|NT DMRs |
| chr16 | 10912139 | 10912855 | 2.06E−07 | 29.61612903 | Hyper-methylated | PCa\|NT DMRs |
| chr16 | 10912902 | 10935122 | 0.000493372 | −23.57431078 | Hypo-methylated | PCa\|NT DMRs |
| chr16 | 10968136 | 10972298 | 0.000847246 | 45.44322344 | Hyper-methylated | PCa\|NT DMRs |
| chr16 | 10973511 | 10974993 | 0.005498599 | 31.77769424 | Hyper-methylated | PCa\|NT DMRs |
| chr16 | 10985729 | 10985903 | 0.000205174 | 41.43154762 | Hyper-methylated | PCa\|NT DMRs |
| chr16 | 11227260 | 11227346 | 0.000205174 | −39.53571429 | Hypo-methylated | PCa\|NT DMRs |
| chr16 | 11282987 | 11283381 | 6.42E−05 | −49.04761905 | Hypo-methylated | PCa\|NT DMRs |
| chr16 | 11304969 | 11305220 | 0.00179283 | −24.47916667 | Hypo-methylated | PCa\|NT DMRs |
| chr16 | 11480649 | 11481343 | 0.001428615 | 20.98958333 | Hyper-methylated | PCa\|NT DMRs |
| chr16 | 11485462 | 11485923 | 0.001969641 | −49.46428571 | Hypo-methylated | PCa\|NT DMRs |
| chr16 | 11519037 | 11524271 | 7.48E−05 | 27.47804233 | Hyper-methylated | PCa\|NT DMRs |
| chr16 | 11763088 | 11764208 | 0.000814525 | 25.01888342 | Hyper-methylated | PCa\|NT DMRs |
| chr16 | 11893815 | 11893874 | 0.005968871 | 50.65714286 | Hyper-methylated | PCa\|NT DMRs |
| chr16 | 12010419 | 12010699 | 6.42E−05 | −43.13492063 | Hypo-methylated | PCa\|NT DMRs |
| chr16 | 12352125 | 12352377 | 0.001181579 | −41.44557823 | Hypo-methylated | PCa\|NT DMRs |
| chr16 | 12355015 | 12355349 | 1.69E−06 | −23.46722689 | Hypo-methylated | PCa\|NT DMRs |
| chr16 | 12992665 | 12994596 | 0.000117699 | 21.57968254 | Hyper-methylated | PCa\|NT DMRs |
| chr16 | 13524375 | 13545035 | 1.76E−06 | −20.1218254 | Hypo-methylated | PCa\|NT DMRs |
| chr16 | 13741886 | 13904843 | 2.62E−05 | −24.95995671 | Hypo-methylated | PCa\|NT DMRs |
| chr16 | 14397001 | 14397993 | 7.93E−05 | 28.99180952 | Hyper-methylated | PCa\|NT DMRs |
| chr16 | 15797238 | 15797298 | 0.005968871 | 26.68571429 | Hyper-methylated | PCa\|NT DMRs |
| chr16 | 15952405 | 15952484 | 5.84E−05 | 24.5952381 | Hyper-methylated | PCa\|NT DMRs |
| chr16 | 16116243 | 16122627 | 0.000972067 | −35.01322751 | Hypo-methylated | PCa\|NT DMRs |
| chr16 | 16693301 | 16730538 | 1.16E−07 | −27.47142857 | Hypo-methylated | PCa\|NT DMRs |
| chr16 | 17107610 | 17107826 | 0.001969641 | 23.53968254 | Hyper-methylated | PCa\|NT DMRs |
| chr16 | 17199250 | 17200244 | 0.006096657 | 20.26587302 | Hyper-methylated | PCa\|NT DMRs |
| chr16 | 17360532 | 17360585 | 0.00064571 | −49.53061224 | Hypo-methylated | PCa\|NT DMRs |
| chr16 | 17699253 | 17813072 | 2.33E−10 | −30.3315873 | Hypo-methylated | PCa\|NT DMRs |
| chr16 | 19030300 | 19041334 | 0.007959104 | −33.31292517 | Hypo-methylated | PCa\|NT DMRs |
| chr16 | 19088006 | 19088442 | 0.003448944 | −41.3877551 | Hypo-methylated | PCa\|NT DMRs |
| chr16 | 19125846 | 19126157 | 5.27E−08 | 24.08823529 | Hyper-methylated | PCa\|NT DMRs |
| chr16 | 19147105 | 19150057 | 0.007391799 | −28.62380952 | Hypo-methylated | PCa\|NT DMRs |
| chr16 | 19568951 | 19570034 | 0.003622801 | −25.61904762 | Hypo-methylated | PCa\|NT DMRs |
| chr16 | 19894307 | 19895579 | 5.30E−12 | 23.46248548 | Hyper-methylated | PCa\|NT DMRs |
| chr16 | 19897013 | 19897156 | 0.001451132 | 21.08051948 | Hyper-methylated | PCa\|NT DMRs |
| chr16 | 20397851 | 20398315 | 0.00324187 | 25.94217687 | Hyper-methylated | PCa\|NT DMRs |
| chr16 | 20923684 | 20923750 | 0.003448944 | 23.53061224 | Hyper-methylated | PCa\|NT DMRs |
| chr16 | 21294947 | 21295120 | 0.000233379 | 36.38571429 | Hyper-methylated | PCa\|NT DMRs |
| chr16 | 21656698 | 21658713 | 0.000260936 | 23.8 | Hyper-methylated | PCa\|NT DMRs |
| chr16 | 22635683 | 22675597 | 0.005646573 | −28.15068027 | Hypo-methylated | PCa\|NT DMRs |
| chr16 | 22745523 | 22745899 | 0.000117699 | −47.20952381 | Hypo-methylated | PCa\|NT DMRs |
| chr16 | 23706211 | 23706538 | 1.68E−05 | 22.70982143 | Hyper-methylated | PCa\|NT DMRs |
| chr16 | 23850114 | 24266921 | 3.40E−18 | −36.39916944 | Hypo-methylated | PCa\|NT DMRs |
| chr16 | 25704220 | 25704401 | 1.17E−08 | 29.75496032 | Hyper-methylated | PCa\|NT DMRs |
| chr16 | 26676580 | 26700624 | 0.000286238 | −32.4014652 | Hypo-methylated | PCa\|NT DMRs |
| chr16 | 27231985 | 27234625 | 0.001083007 | −40.84285714 | Hypo-methylated | PCa\|NT DMRs |
| chr16 | 27237991 | 27238100 | 6.42E−05 | −33.22539683 | Hypo-methylated | PCa\|NT DMRs |
| chr16 | 27243976 | 27244604 | 5.94E−06 | −41.46493506 | Hypo-methylated | PCa\|NT DMRs |
| chr16 | 27325893 | 27327315 | 0.003871035 | 25.43452381 | Hyper-methylated | PCa\|NT DMRs |
| chr16 | 27330097 | 27330246 | 0.00064571 | 59.93197279 | Hyper-methylated | PCa\|NT DMRs |
| chr16 | 27445366 | 27445781 | 0.005674668 | −31.94557823 | Hypo-methylated | PCa\|NT DMRs |
| chr16 | 27475091 | 27475140 | 0.008234951 | 23.36 | Hyper-methylated | PCa\|NT DMRs |
| chr16 | 27510503 | 27511270 | 0.000514174 | −31.33766234 | Hypo-methylated | PCa\|NT DMRs |
| chr16 | 27791162 | 27791356 | 4.08E−06 | −25.5327381 | Hypo-methylated | PCa\|NT DMRs |
| chr16 | 27792769 | 27793304 | 0.00068166 | −44.05357143 | Hypo-methylated | PCa\|NT DMRs |
| chr16 | 27984243 | 28014977 | 0.004691973 | −22.82993197 | Hypo-methylated | PCa\|NT DMRs |
| chr16 | 28312542 | 28321579 | 0.002534928 | 28.41428571 | Hyper-methylated | PCa\|NT DMRs |
| chr16 | 28505826 | 28506118 | 0.005968871 | 50.5952381 | Hyper-methylated | PCa\|NT DMRs |
| chr16 | 28549267 | 28550794 | 5.19E−07 | −59.83113553 | Hypo-methylated | PCa\|NT DMRs |
| chr16 | 28555134 | 28564807 | 0.003448944 | −32.83265306 | Hypo-methylated | PCa\|NT DMRs |
| chr16 | 28750459 | 28758726 | 0.000339627 | 21.16309524 | Hyper-methylated | PCa\|NT DMRs |
| chr16 | 28893790 | 28894485 | 4.11E−06 | −34.03063492 | Hypo-methylated | PCa\|NT DMRs |
| chr16 | 28914467 | 28914660 | 0.004095673 | 38.69206349 | Hyper-methylated | PCa\|NT DMRs |
| chr16 | 28995537 | 28999703 | 5.37E−05 | 46.23952381 | Hyper-methylated | PCa\|NT DMRs |
| chr16 | 29030346 | 29031714 | 0.000205174 | −21.32440476 | Hypo-methylated | PCa\|NT DMRs |
| chr16 | 29086221 | 29086283 | 0.00023371 | 45.45714286 | Hyper-methylated | PCa\|NT DMRs |
| chr16 | 29118855 | 29118998 | 0.002373155 | −39.82539683 | Hypo-methylated | PCa\|NT DMRs |
| chr16 | 29140241 | 29159108 | 2.15E−11 | −25.04742468 | Hypo-methylated | PCa\|NT DMRs |
| chr16 | 29160196 | 29160273 | 0.00064571 | 39.69387755 | Hyper-methylated | PCa\|NT DMRs |
| chr16 | 29170156 | 29189484 | 4.92E−13 | −25.3822619 | Hypo-methylated | PCa\|NT DMRs |
| chr16 | 29191465 | 29193962 | 4.20E−07 | −22.00910973 | Hypo-methylated | PCa\|NT DMRs |
| chr16 | 29229209 | 29268470 | 8.33E−29 | −23.31564073 | Hypo-methylated | PCa\|NT DMRs |
| chr16 | 29697695 | 29701116 | 0.004553481 | 34.11904762 | Hyper-methylated | PCa\|NT DMRs |
| chr16 | 29755503 | 29758784 | 1.96E−05 | 27.1875 | Hyper-methylated | PCa\|NT DMRs |
| chr16 | 29830887 | 29831040 | 2.06E−05 | −33.41836735 | Hypo-methylated | PCa\|NT DMRs |
| chr16 | 29868727 | 29870508 | 0.000483473 | −20.96390977 | Hypo-methylated | PCa\|NT DMRs |
| chr16 | 29887710 | 29890333 | 3.49E−12 | 36.93904762 | Hyper-methylated | PCa\|NT DMRs |
| chr16 | 30034006 | 30036062 | 0.000836132 | −27.81746032 | Hypo-methylated | PCa\|NT DMRs |
| chr16 | 30066508 | 30066606 | 0.001969641 | 37.97619048 | Hyper-methylated | PCa\|NT DMRs |
| chr16 | 30103997 | 30104150 | 0.001115302 | 25.69642857 | Hyper-methylated | PCa\|NT DMRs |
| chr16 | 30370636 | 30375183 | 1.42E−05 | −20.76857143 | Hypo-methylated | PCa\|NT DMRs |
| chr16 | 30485053 | 30485425 | 3.61E−05 | 38.51870748 | Hyper-methylated | PCa\|NT DMRs |
| chr16 | 30567234 | 30567586 | 0.001344447 | −39.85873016 | Hypo-methylated | PCa\|NT DMRs |
| chr16 | 30572702 | 30573334 | 2.01E−09 | 22.61778976 | Hyper-methylated | PCa\|NT DMRs |
| chr16 | 30660787 | 30661577 | 0.002437742 | 35.36772487 | Hyper-methylated | PCa\|NT DMRs |
| chr16 | 30736144 | 30736666 | 8.96E−05 | 25.78061224 | Hyper-methylated | PCa\|NT DMRs |
| chr16 | 30907606 | 30908535 | 2.17E−10 | 23.55592186 | Hyper-methylated | PCa\|NT DMRs |
| chr16 | 30914154 | 30915889 | 0.004553481 | 34.89417989 | Hyper-methylated | PCa\|NT DMRs |
| chr16 | 30976187 | 30976274 | 0.005968871 | 37.92 | Hyper-methylated | PCa\|NT DMRs |
| chr16 | 31004398 | 31004456 | 0.003871035 | 32.125 | Hyper-methylated | PCa\|NT DMRs |
| chr16 | 31013289 | 31020068 | 0.002193447 | −23.295671 | Hypo-methylated | PCa\|NT DMRs |
| chr16 | 31075643 | 31075742 | 0.000652677 | 48.67222222 | Hyper-methylated | PCa\|NT DMRs |
| chr16 | 31120626 | 31120674 | 0.003871035 | −24.89285714 | Hypo-methylated | PCa\|NT DMRs |
| chr16 | 31141563 | 31141817 | 0.001115302 | 23.19642857 | Hyper-methylated | PCa\|NT DMRs |
| chr16 | 31226074 | 31226186 | 0.00023371 | 27.26666667 | Hyper-methylated | PCa\|NT DMRs |
| chr16 | 31271642 | 31271992 | 0.001969641 | 38.81984127 | Hyper-methylated | PCa\|NT DMRs |
| chr16 | 31276614 | 31276838 | 0.00641359 | 20.54761905 | Hyper-methylated | PCa\|NT DMRs |
| chr16 | 31282969 | 31341899 | 8.62E−12 | −24.68791756 | Hypo-methylated | PCa\|NT DMRs |
| chr16 | 31363703 | 31363807 | 0.003622801 | 28.94761905 | Hyper-methylated | PCa\|NT DMRs |
| chr16 | 31368394 | 31368868 | 0.009924186 | 20.1952381 | Hyper-methylated | PCa\|NT DMRs |
| chr16 | 31548681 | 31549142 | 5.49E−13 | 22.38095238 | Hyper-methylated | PCa\|NT DMRs |
| chr16 | 31590383 | 31709112 | 4.30E−08 | −31.54642857 | Hypo-methylated | PCa\|NT DMRs |
| chr16 | 32297137 | 32300810 | 0.000117699 | −29.54588745 | Hypo-methylated | PCa\|NT DMRs |
| chr16 | 32338210 | 32352520 | 0.002048317 | −27.125 | Hypo-methylated | PCa\|NT DMRs |
| chr16 | 32640501 | 32645798 | 0.00379014 | −34.72916667 | Hypo-methylated | PCa\|NT DMRs |
| chr16 | 33237962 | 33298210 | 1.21E−06 | −27.73581781 | Hypo-methylated | PCa\|NT DMRs |
| chr16 | 33406088 | 33483659 | 2.02E−09 | −30.97863578 | Hypo-methylated | PCa\|NT DMRs |
| chr16 | 33944442 | 33949690 | 1.62E−05 | −22.26060606 | Hypo-methylated | PCa\|NT DMRs |
| chr16 | 33955811 | 33956699 | 0.005968871 | −23.84415584 | Hypo-methylated | PCa\|NT DMRs |
| chr16 | 33958841 | 33959929 | 0.000232782 | −25.0702381 | Hypo-methylated | PCa\|NT DMRs |
| chr16 | 46765374 | 46765466 | 0.008482405 | −53.82285714 | Hypo-methylated | PCa\|NT DMRs |
| chr16 | 46791236 | 46791494 | 0.001969641 | −46.7755102 | Hypo-methylated | PCa\|NT DMRs |
| chr16 | 46878314 | 46878490 | 7.15E−06 | 27.615 | Hyper-methylated | PCa\|NT DMRs |
| chr16 | 46919004 | 46921305 | 0.003535943 | −20.41552028 | Hypo-methylated | PCa\|NT DMRs |
| chr16 | 46945625 | 46956365 | 1.20E−05 | −46.18095238 | Hypo-methylated | PCa\|NT DMRs |
| chr16 | 48093524 | 48094446 | 2.41E−08 | −29.40062112 | Hypo-methylated | PCa\|NT DMRs |
| chr16 | 48641898 | 48643082 | 0.001237229 | −27.32261905 | Hypo-methylated | PCa\|NT DMRs |
| chr16 | 48644653 | 48644724 | 0.005513661 | −20.06845238 | Hypo-methylated | PCa\|NT DMRs |
| chr16 | 48663320 | 48663544 | 0.000215808 | −39.88095238 | Hypo-methylated | PCa\|NT DMRs |
| chr16 | 48665375 | 48733811 | 0.001241245 | −34.85873016 | Hypo-methylated | PCa\|NT DMRs |
| chr16 | 49525295 | 49525795 | 0.001563885 | −30.705 | Hypo-methylated | PCa\|NT DMRs |
| chr16 | 49537207 | 49537395 | 0.001969641 | 37.54761905 | Hyper-methylated | PCa\|NT DMRs |
| chr16 | 49758531 | 49764953 | 1.44E−06 | −25.93109244 | Hypo-methylated | PCa\|NT DMRs |
| chr16 | 49776723 | 49800714 | 0.00046742 | −23.87662338 | Hypo-methylated | PCa\|NT DMRs |
| chr16 | 49911050 | 49930277 | 0.001993134 | −20.01515152 | Hypo-methylated | PCa\|NT DMRs |
| chr16 | 50280386 | 50280724 | 0.00068008 | 28.49548872 | Hyper-methylated | PCa\|NT DMRs |
| chr16 | 50301976 | 50318037 | 1.45E−07 | 42.65498866 | Hyper-methylated | PCa\|NT DMRs |
| chr16 | 50715181 | 50720356 | 0.001139334 | 36.15079365 | Hyper-methylated | PCa\|NT DMRs |
| chr16 | 50875166 | 50875609 | 4.35E−06 | 20.3987013 | Hyper-methylated | PCa\|NT DMRs |
| chr16 | 51467507 | 51591255 | 7.09E−05 | −25.65057471 | Hypo-methylated | PCa\|NT DMRs |
| chr16 | 52581377 | 52589163 | 2.42E−06 | 31.60547619 | Hyper-methylated | PCa\|NT DMRs |
| chr16 | 52655635 | 52655804 | 0.001969641 | −34.19206349 | Hypo-methylated | PCa\|NT DMRs |
| chr16 | 54209645 | 54210071 | 0.00064571 | 20.82312925 | Hyper-methylated | PCa\|NT DMRs |
| chr16 | 54268993 | 54269138 | 0.005968871 | 40.48571429 | Hyper-methylated | PCa\|NT DMRs |
| chr16 | 54465950 | 54467538 | 8.94E−05 | 31.65162907 | Hyper-methylated | PCa\|NT DMRs |
| chr16 | 54851427 | 54851727 | 0.007347803 | −24.80952381 | Hypo-methylated | PCa\|NT DMRs |
| chr16 | 55040809 | 55079401 | 1.54E−12 | −23.83809524 | Hypo-methylated | PCa\|NT DMRs |
| chr16 | 55290536 | 55297451 | 2.42E−08 | −32.2672619 | Hypo-methylated | PCa\|NT DMRs |
| chr16 | 55425192 | 55504853 | 3.58E−07 | −31.55192744 | Hypo-methylated | PCa\|NT DMRs |
| chr16 | 55519607 | 55522628 | 0.000205174 | −24.08333333 | Hypo-methylated | PCa\|NT DMRs |
| chr16 | 56362672 | 56362957 | 5.71E−06 | −20.22321429 | Hypo-methylated | PCa\|NT DMRs |
| chr16 | 56385026 | 56386186 | 0.001969641 | −20.17460317 | Hypo-methylated | PCa\|NT DMRs |
| chr16 | 56661082 | 56661690 | 0.005513661 | −43.98027211 | Hypo-methylated | PCa\|NT DMRs |
| chr16 | 56668846 | 56673868 | 1.03E−24 | 34.13711002 | Hyper-methylated | PCa\|NT DMRs |
| chr16 | 56681703 | 56682206 | 2.49E−05 | 23.73764881 | Hyper-methylated | PCa\|NT DMRs |
| chr16 | 56696592 | 56697034 | 3.11E−06 | 33.57261905 | Hyper-methylated | PCa\|NT DMRs |
| chr16 | 56703388 | 56704438 | 1.73E−10 | 31.3697995 | Hyper-methylated | PCa\|NT DMRs |
| chr16 | 56709403 | 56715456 | 0.000143323 | 26.59255189 | Hyper-methylated | PCa\|NT DMRs |
| chr16 | 56892413 | 56892547 | 6.93E−06 | 52.45714286 | Hyper-methylated | PCa\|NT DMRs |
| chr16 | 57311903 | 57318285 | 0.001651534 | 20.79831029 | Hyper-methylated | PCa\|NT DMRs |
| chr16 | 57352141 | 57373846 | 0.007959104 | −35.28911565 | Hypo-methylated | PCa\|NT DMRs |
| chr16 | 57509003 | 57510004 | 0.000122432 | 35.05194805 | Hyper-methylated | PCa\|NT DMRs |
| chr16 | 57512940 | 57513297 | 2.87E−06 | 30.00661376 | Hyper-methylated | PCa\|NT DMRs |
| chr16 | 57519833 | 57520015 | 2.85E−05 | 27.35714286 | Hyper-methylated | PCa\|NT DMRs |
| chr16 | 57578985 | 57605309 | 0.00023371 | −27.97190476 | Hypo-methylated | PCa\|NT DMRs |
| chr16 | 57648940 | 57657248 | 1.34E−08 | −39.91803752 | Hypo-methylated | PCa\|NT DMRs |
| chr16 | 57659254 | 57659440 | 0.001969641 | 52.95634921 | Hyper-methylated | PCa\|NT DMRs |
| chr16 | 57662166 | 57697216 | 2.15E−06 | 29.26357143 | Hyper-methylated | PCa\|NT DMRs |
| chr16 | 57828807 | 57832292 | 5.71E−10 | −35.82578397 | Hypo-methylated | PCa\|NT DMRs |
| chr16 | 58018440 | 58018757 | 8.75E−05 | 24.15079365 | Hyper-methylated | PCa\|NT DMRs |
| chr16 | 58115573 | 58115754 | 0.004095673 | 40.5952381 | Hyper-methylated | PCa\|NT DMRs |
| chr16 | 58148917 | 58149526 | 0.000570576 | −24.34920635 | Hypo-methylated | PCa\|NT DMRs |
| chr16 | 58880322 | 59494155 | 2.60E−19 | −34.49847375 | Hypo-methylated | PCa\|NT DMRs |
| chr16 | 59554848 | 59788978 | 3.75E−12 | −30.41439909 | Hypo-methylated | PCa\|NT DMRs |
| chr16 | 62485889 | 62682151 | 2.01E−07 | −36.32928571 | Hypo-methylated | PCa\|NT DMRs |
| chr16 | 62758889 | 62992823 | 3.46E−15 | −28.91492674 | Hypo-methylated | PCa\|NT DMRs |
| chr16 | 66360704 | 66361203 | 1.76E−06 | −47.7218254 | Hypo-methylated | PCa\|NT DMRs |
| chr16 | 66436624 | 66437053 | 7.33E−10 | −21.65894466 | Hypo-methylated | PCa\|NT DMRs |
| chr16 | 66461441 | 66462196 | 9.31E−10 | 22.17904762 | Hyper-methylated | PCa\|NT DMRs |
| chr16 | 66969363 | 66969606 | 2.18E−06 | −36.13784461 | Hypo-methylated | PCa\|NT DMRs |
| chr16 | 66982435 | 66982944 | 5.37E−09 | 33.00549451 | Hyper-methylated | PCa\|NT DMRs |
| chr16 | 67184052 | 67188775 | 4.76E−17 | 23.42979094 | Hyper-methylated | PCa\|NT DMRs |
| chr16 | 67195270 | 67196089 | 0.003764982 | −23.73809524 | Hypo-methylated | PCa\|NT DMRs |
| chr16 | 67196900 | 67204509 | 2.41E−28 | 31.29674185 | Hyper-methylated | PCa\|NT DMRs |
| chr16 | 67208800 | 67209081 | 0.001969641 | 20.58095238 | Hyper-methylated | PCa\|NT DMRs |
| chr16 | 67226937 | 67234153 | 1.09E−16 | −38.08596491 | Hypo-methylated | PCa\|NT DMRs |
| chr16 | 67241436 | 67241710 | 0.000205174 | −51.98214286 | Hypo-methylated | PCa\|NT DMRs |
| chr16 | 67318599 | 67319292 | 0.000564835 | −36.59340659 | Hypo-methylated | PCa\|NT DMRs |
| chr16 | 67335510 | 67335792 | 0.00064571 | −50.74829932 | Hypo-methylated | PCa\|NT DMRs |
| chr16 | 67427105 | 67429065 | 1.41E−23 | 28.25015576 | Hyper-methylated | PCa\|NT DMRs |
| chr16 | 67475109 | 67481530 | 0.000580099 | −35.4929972 | Hypo-methylated | PCa\|NT DMRs |
| chr16 | 67552676 | 67552835 | 0.005968871 | 51.5952381 | Hyper-methylated | PCa\|NT DMRs |
| chr16 | 67678102 | 67679557 | 4.59E−10 | 20.634 | Hyper-methylated | PCa\|NT DMRs |
| chr16 | 67682065 | 67688497 | 5.32E−23 | −33.09000249 | Hypo-methylated | PCa\|NT DMRs |
| chr16 | 67701162 | 67703106 | 0.001391421 | 28.17662338 | Hyper-methylated | PCa\|NT DMRs |
| chr16 | 67916426 | 67918902 | 1.56E−05 | −43.96492063 | Hypo-methylated | PCa\|NT DMRs |
| chr16 | 67960491 | 67960722 | 0.000377317 | −35.28571429 | Hypo-methylated | PCa\|NT DMRs |
| chr16 | 67967146 | 67969343 | 0.001083007 | −42.13142857 | Hypo-methylated | PCa\|NT DMRs |
| chr16 | 68001760 | 68002208 | 1.34E−05 | 24.76693122 | Hyper-methylated | PCa\|NT DMRs |
| chr16 | 68035220 | 68053960 | 6.69E−05 | −26.6047619 | Hypo-methylated | PCa\|NT DMRs |
| chr16 | 68109138 | 68119017 | 1.09E−07 | 36.05064935 | Hyper-methylated | PCa\|NT DMRs |
| chr16 | 68265576 | 68271046 | 8.06E−08 | −22.62292627 | Hypo-methylated | PCa\|NT DMRs |
| chr16 | 68385269 | 68385717 | 5.84E−05 | −39.30555556 | Hypo-methylated | PCa\|NT DMRs |
| chr16 | 68389417 | 68390717 | 4.25E−06 | 40.31878307 | Hyper-methylated | PCa\|NT DMRs |
| chr16 | 68411847 | 68412150 | 0.00641359 | −34.96428571 | Hypo-methylated | PCa\|NT DMRs |
| chr16 | 68670007 | 68670380 | 0.000205174 | −52.42261905 | Hypo-methylated | PCa\|NT DMRs |
| chr16 | 68676215 | 68678616 | 3.89E−23 | 37.88924162 | Hyper-methylated | PCa\|NT DMRs |
| chr16 | 69141067 | 69142170 | 4.52E−14 | 27.99831933 | Hyper-methylated | PCa\|NT DMRs |
| chr16 | 69895355 | 69896037 | 0.007959104 | −37.65646259 | Hypo-methylated | PCa\|NT DMRs |
| chr16 | 69971422 | 69971565 | 0.001583444 | −21.31428571 | Hypo-methylated | PCa\|NT DMRs |
| chr16 | 70222533 | 70222612 | 0.000474737 | 29.1962963 | Hyper-methylated | PCa\|NT DMRs |
| chr16 | 70321963 | 70322213 | 0.000205174 | −44.46130952 | Hypo-methylated | PCa\|NT DMRs |
| chr16 | 70381845 | 70381893 | 0.005968871 | −26.82857143 | Hypo-methylated | PCa\|NT DMRs |
| chr16 | 70457082 | 70460575 | 0.005674668 | 31.61020408 | Hyper-methylated | PCa\|NT DMRs |
| chr16 | 70633352 | 70642101 | 0.002767976 | −23.35714286 | Hypo-methylated | PCa\|NT DMRs |
| chr16 | 70646782 | 70647416 | 0.003689112 | 33.99267399 | Hyper-methylated | PCa\|NT DMRs |
| chr16 | 71407458 | 71414373 | 6.69E−05 | −22.52571429 | Hypo-methylated | PCa\|NT DMRs |
| chr16 | 71465153 | 71467260 | 0.003871035 | −30.39285714 | Hypo-methylated | PCa\|NT DMRs |
| chr16 | 71844062 | 71845892 | 0.002767976 | 26.05800866 | Hyper-methylated | PCa\|NT DMRs |
| chr16 | 72661210 | 72661480 | 0.00023371 | −35.65714286 | Hypo-methylated | PCa\|NT DMRs |
| chr16 | 72940258 | 72940306 | 0.001969641 | 23.39285714 | Hyper-methylated | PCa\|NT DMRs |
| chr16 | 73461766 | 73514946 | 6.85E−06 | −25.06582633 | Hypo-methylated | PCa\|NT DMRs |
| chr16 | 73716406 | 73716450 | 0.005968871 | −21.05238095 | Hypo-methylated | PCa\|NT DMRs |
| chr16 | 73900357 | 73900614 | 0.005968871 | −45.20380952 | Hypo-methylated | PCa\|NT DMRs |
| chr16 | 74030719 | 74064139 | 0.000205174 | −25.10714286 | Hypo-methylated | PCa\|NT DMRs |
| chr16 | 74411948 | 74433121 | 0.002826041 | −26.2755102 | Hypo-methylated | PCa\|NT DMRs |
| chr16 | 74777837 | 74778058 | 0.001969641 | −64.62517007 | Hypo-methylated | PCa\|NT DMRs |
| chr16 | 75256329 | 75261099 | 0.000438659 | −28.59761905 | Hypo-methylated | PCa\|NT DMRs |
| chr16 | 75279396 | 75282362 | 0.003391914 | −29.32592593 | Hypo-methylated | PCa\|NT DMRs |
| chr16 | 75540559 | 75540708 | 0.005968871 | 25.42857143 | Hyper-methylated | PCa\|NT DMRs |
| chr16 | 77228648 | 77228980 | 5.84E−05 | −29.325 | Hypo-methylated | PCa\|NT DMRs |
| chr16 | 77369704 | 77392244 | 2.79E−05 | −27.3047619 | Hypo-methylated | PCa\|NT DMRs |
| chr16 | 77895862 | 77918602 | 0.00179283 | −25.95454545 | Hypo-methylated | PCa\|NT DMRs |
| chr16 | 79253101 | 79287222 | 0.001060348 | −23.83982684 | Hypo-methylated | PCa\|NT DMRs |
| chr16 | 79463826 | 79464272 | 0.00064571 | −41.01496599 | Hypo-methylated | PCa\|NT DMRs |
| chr16 | 79553532 | 79553817 | 0.001076343 | 33.49350649 | Hyper-methylated | PCa\|NT DMRs |
| chr16 | 81270959 | 81272783 | 0.005968871 | −60.49142857 | Hypo-methylated | PCa\|NT DMRs |
| chr16 | 81434752 | 81435017 | 0.000205174 | −54.74642857 | Hypo-methylated | PCa\|NT DMRs |
| chr16 | 81490286 | 81506999 | 0.00641359 | 20.28698413 | Hyper-methylated | PCa\|NT DMRs |
| chr16 | 81528194 | 81529074 | 0.000117699 | 44.24935065 | Hyper-methylated | PCa\|NT DMRs |
| chr16 | 81552099 | 81554825 | 0.001111028 | 20.4042328 | Hyper-methylated | PCa\|NT DMRs |
| chr16 | 81580631 | 81585567 | 0.000783791 | 21.05238095 | Hyper-methylated | PCa\|NT DMRs |
| chr16 | 81632836 | 81640483 | 0.000754912 | −33.94984127 | Hypo-methylated | PCa\|NT DMRs |
| chr16 | 81776981 | 81778849 | 0.007959104 | −24.16122449 | Hypo-methylated | PCa\|NT DMRs |
| chr16 | 81832432 | 81836220 | 0.005968871 | 39.90793651 | Hyper-methylated | PCa\|NT DMRs |
| chr16 | 83442827 | 83497974 | 1.85E−06 | −28.03781513 | Hypo-methylated | PCa\|NT DMRs |
| chr16 | 83655339 | 83728737 | 4.93E−07 | −24.46666667 | Hypo-methylated | PCa\|NT DMRs |
| chr16 | 83914357 | 83916269 | 0.000205174 | −24.51309524 | Hypo-methylated | PCa\|NT DMRs |
| chr16 | 84057355 | 84057681 | 0.000215808 | −26.58730159 | Hypo-methylated | PCa\|NT DMRs |
| chr16 | 84078931 | 84079123 | 0.002094 | 29.6452381 | Hyper-methylated | PCa\|NT DMRs |
| chr16 | 84327616 | 84328392 | 0.000371883 | 32.96642857 | Hyper-methylated | PCa\|NT DMRs |
| chr16 | 84335171 | 84336770 | 0.000170278 | −20.21428571 | Hypo-methylated | PCa\|NT DMRs |
| chr16 | 84390197 | 84390468 | 0.005968871 | −50.21428571 | Hypo-methylated | PCa\|NT DMRs |
| chr16 | 84441716 | 84441780 | 0.008482405 | −51.25714286 | Hypo-methylated | PCa\|NT DMRs |
| chr16 | 84578282 | 84578376 | 0.005968871 | 42.14761905 | Hyper-methylated | PCa\|NT DMRs |
| chr16 | 84763185 | 84771552 | 0.006170736 | −27.19421769 | Hypo-methylated | PCa\|NT DMRs |
| chr16 | 84819552 | 84819620 | 0.00068166 | −29.69642857 | Hypo-methylated | PCa\|NT DMRs |
| chr16 | 84860443 | 84860841 | 0.001789867 | 22.75132275 | Hyper-methylated | PCa\|NT DMRs |
| chr16 | 84861455 | 84861803 | 3.44E−06 | −47.92857143 | Hypo-methylated | PCa\|NT DMRs |
| chr16 | 84876118 | 84876862 | 6.69E−05 | −38.13333333 | Hypo-methylated | PCa\|NT DMRs |
| chr16 | 84918823 | 84919169 | 0.000205174 | 40.95178571 | Hyper-methylated | PCa\|NT DMRs |
| chr16 | 84943821 | 84950077 | 0.000296718 | 27.55646259 | Hyper-methylated | PCa\|NT DMRs |
| chr16 | 85110644 | 85112489 | 0.00046742 | 37.60779221 | Hyper-methylated | PCa\|NT DMRs |
| chr16 | 85116332 | 85119269 | 6.51E−07 | 51.89104762 | Hyper-methylated | PCa\|NT DMRs |
| chr16 | 85168443 | 85170151 | 6.69E−05 | 31.93854875 | Hyper-methylated | PCa\|NT DMRs |
| chr16 | 85183518 | 85183685 | 0.005968871 | −56.57142857 | Hypo-methylated | PCa\|NT DMRs |
| chr16 | 85203608 | 85203940 | 1.69E−06 | 25.3226601 | Hyper-methylated | PCa\|NT DMRs |
| chr16 | 85271771 | 85272063 | 0.000294557 | 22.48484848 | Hyper-methylated | PCa\|NT DMRs |
| chr16 | 85292690 | 85296087 | 5.84E−05 | 48.92420635 | Hyper-methylated | PCa\|NT DMRs |
| chr16 | 85337819 | 85349956 | 1.30E−06 | 32.44621849 | Hyper-methylated | PCa\|NT DMRs |
| chr16 | 85361134 | 85363063 | 0.001083007 | 30.84285714 | Hyper-methylated | PCa\|NT DMRs |
| chr16 | 85388137 | 85388729 | 0.001115302 | 33.05952381 | Hyper-methylated | PCa\|NT DMRs |
| chr16 | 85418540 | 85418838 | 0.000652677 | −28.44047619 | Hypo-methylated | PCa\|NT DMRs |
| chr16 | 85418881 | 85422836 | 0.001866471 | 32.5242236 | Hyper-methylated | PCa\|NT DMRs |
| chr16 | 85429386 | 85429570 | 0.009106413 | 27.98412698 | Hyper-methylated | PCa\|NT DMRs |
| chr16 | 85495448 | 85495725 | 0.00064571 | 44.31292517 | Hyper-methylated | PCa\|NT DMRs |
| chr16 | 85517185 | 85518386 | 6.42E−05 | −33.19047619 | Hypo-methylated | PCa\|NT DMRs |
| chr16 | 85522260 | 85522512 | 0.001969641 | 22.00396825 | Hyper-methylated | PCa\|NT DMRs |
| chr16 | 85551958 | 85552154 | 0.000205174 | 24.38392857 | Hyper-methylated | PCa\|NT DMRs |
| chr16 | 85554650 | 85557931 | 2.26E−06 | −36.81547619 | Hypo-methylated | PCa\|NT DMRs |
| chr16 | 85600230 | 85607809 | 5.84E−06 | 24.21606134 | Hyper-methylated | PCa\|NT DMRs |
| chr16 | 85619999 | 85620238 | 7.57E−05 | −43.33333333 | Hypo-methylated | PCa\|NT DMRs |
| chr16 | 85741402 | 85741555 | 1.97E−05 | −53.34 | Hypo-methylated | PCa\|NT DMRs |
| chr16 | 85783538 | 85784193 | 0.00051204 | 43.96285714 | Hyper-methylated | PCa\|NT DMRs |
| chr16 | 85816316 | 85816992 | 0.003871035 | 34.9797619 | Hyper-methylated | PCa\|NT DMRs |
| chr16 | 85981833 | 85982028 | 0.000205174 | 32.79761905 | Hyper-methylated | PCa\|NT DMRs |
| chr16 | 85982195 | 86005909 | 1.70E−06 | −27.07301587 | Hypo-methylated | PCa\|NT DMRs |
| chr16 | 86605190 | 86607704 | 5.99E−05 | −25.96630037 | Hypo-methylated | PCa\|NT DMRs |
| chr16 | 86611303 | 86611892 | 0.000215808 | −24.44444444 | Hypo-methylated | PCa\|NT DMRs |
| chr16 | 86625460 | 86629385 | 4.73E−05 | −36.4360119 | Hypo-methylated | PCa\|NT DMRs |
| chr16 | 86962224 | 86963104 | 8.82E−07 | 20.72419562 | Hyper-methylated | PCa\|NT DMRs |
| chr16 | 87086835 | 87087126 | 8.28E−07 | 25.81746032 | Hyper-methylated | PCa\|NT DMRs |
| chr16 | 87312849 | 87313133 | 0.00641359 | −21.81746032 | Hypo-methylated | PCa\|NT DMRs |
| chr16 | 87358571 | 87358621 | 0.00064571 | 40.88095238 | Hyper-methylated | PCa\|NT DMRs |
| chr16 | 87417801 | 87418310 | 0.000117986 | 27.59821429 | Hyper-methylated | PCa\|NT DMRs |
| chr16 | 87527371 | 87531345 | 9.23E−05 | −46.22745098 | Hypo-methylated | PCa\|NT DMRs |
| chr16 | 87545844 | 87550120 | 6.42E−05 | −21.22486772 | Hypo-methylated | PCa\|NT DMRs |
| chr16 | 87552012 | 87552511 | 0.00023371 | −24.4965368 | Hypo-methylated | PCa\|NT DMRs |
| chr16 | 87569839 | 87572637 | 0.005968871 | −33.61714286 | Hypo-methylated | PCa\|NT DMRs |
| chr16 | 87624313 | 87628248 | 3.10E−09 | −28.4764411 | Hypo-methylated | PCa\|NT DMRs |
| chr16 | 87671820 | 87675538 | 5.78E−05 | −26.32380952 | Hypo-methylated | PCa\|NT DMRs |
| chr16 | 87681596 | 87682204 | 2.82E−10 | −26.15873016 | Hypo-methylated | PCa\|NT DMRs |
| chr16 | 87690069 | 87690138 | 0.002097193 | −33.67346939 | Hypo-methylated | PCa\|NT DMRs |
| chr16 | 87717884 | 87718134 | 1.42E−05 | −47.05952381 | Hypo-methylated | PCa\|NT DMRs |
| chr16 | 87720606 | 87720767 | 0.007959104 | −29.95918367 | Hypo-methylated | PCa\|NT DMRs |
| chr16 | 87842167 | 87843352 | 0.002097193 | 37.21972789 | Hyper-methylated | PCa\|NT DMRs |
| chr16 | 87850009 | 87850371 | 1.97E−05 | 34.11428571 | Hyper-methylated | PCa\|NT DMRs |
| chr16 | 87852974 | 87853710 | 1.11E−05 | −26.4025974 | Hypo-methylated | PCa\|NT DMRs |
| chr16 | 87857290 | 87860884 | 1.12E−09 | −22.9912892 | Hypo-methylated | PCa\|NT DMRs |
| chr16 | 87942514 | 87945159 | 3.89E−05 | −50.69047619 | Hypo-methylated | PCa\|NT DMRs |
| chr16 | 87979181 | 87979417 | 0.00064571 | −20.81292517 | Hypo-methylated | PCa\|NT DMRs |
| chr16 | 87988087 | 88000776 | 4.79E−05 | 31.91991342 | Hyper-methylated | PCa\|NT DMRs |
| chr16 | 88111053 | 88112406 | 0.003662905 | −29.78095238 | Hypo-methylated | PCa\|NT DMRs |
| chr16 | 88123428 | 88124183 | 0.000122432 | 34.28311688 | Hyper-methylated | PCa\|NT DMRs |
| chr16 | 88279309 | 88279491 | 0.004095673 | −43.07142857 | Hypo-methylated | PCa\|NT DMRs |
| chr16 | 88454593 | 88455226 | 6.52E−10 | −47.8218126 | Hypo-methylated | PCa\|NT DMRs |
| chr16 | 88499240 | 88501573 | 1.85E−10 | −25.72777778 | Hypo-methylated | PCa\|NT DMRs |
| chr16 | 88503436 | 88503524 | 0.000205174 | −45.14880952 | Hypo-methylated | PCa\|NT DMRs |
| chr16 | 88503611 | 88505415 | 2.55E−06 | −23.79708141 | Hypo-methylated | PCa\|NT DMRs |
| chr16 | 88518782 | 88518822 | 0.005968871 | 43.31428571 | Hyper-methylated | PCa\|NT DMRs |
| chr16 | 88535803 | 88537416 | 0.00044133 | 24.84081633 | Hyper-methylated | PCa\|NT DMRs |
| chr16 | 88543358 | 88545582 | 0.000713083 | 20.70680272 | Hyper-methylated | PCa\|NT DMRs |
| chr16 | 88565468 | 88566700 | 4.74E−10 | 32.31785714 | Hyper-methylated | PCa\|NT DMRs |
| chr16 | 88575736 | 88576721 | 1.02E−05 | −29.54880952 | Hypo-methylated | PCa\|NT DMRs |
| chr16 | 88594340 | 88594529 | 0.001610287 | −21.6875 | Hypo-methylated | PCa\|NT DMRs |
| chr16 | 88615339 | 88615891 | 8.93E−05 | −27.7264881 | Hypo-methylated | PCa\|NT DMRs |
| chr16 | 88619410 | 88619601 | 0.005968871 | 27.71428571 | Hyper-methylated | PCa\|NT DMRs |
| chr16 | 88637645 | 88638000 | 0.006225823 | −27.99350649 | Hypo-methylated | PCa\|NT DMRs |
| chr16 | 88665986 | 88666412 | 6.17E−06 | 28.94246032 | Hyper-methylated | PCa\|NT DMRs |
| chr16 | 88715958 | 88719086 | 9.26E−14 | 39.17441193 | Hyper-methylated | PCa\|NT DMRs |
| chr16 | 88757871 | 88760007 | 0.000734562 | 30.43877551 | Hyper-methylated | PCa\|NT DMRs |
| chr16 | 88765156 | 88771255 | 3.42E−18 | 40.06059415 | Hyper-methylated | PCa\|NT DMRs |
| chr16 | 88794490 | 88794625 | 0.001969641 | −50.24489796 | Hypo-methylated | PCa\|NT DMRs |
| chr16 | 88812181 | 88812639 | 1.76E−06 | 54.7952381 | Hyper-methylated | PCa\|NT DMRs |
| chr16 | 88821701 | 88822748 | 0.000951499 | −28.6 | Hypo-methylated | PCa\|NT DMRs |
| chr16 | 88844378 | 88845595 | 0.000247245 | 29.73214286 | Hyper-methylated | PCa\|NT DMRs |
| chr16 | 88853243 | 88860289 | 3.85E−06 | 33.03519164 | Hyper-methylated | PCa\|NT DMRs |
| chr16 | 88865937 | 88869438 | 4.61E−07 | 38.35277778 | Hyper-methylated | PCa\|NT DMRs |
| chr16 | 88910430 | 88910690 | 0.000205174 | −45.89285714 | Hypo-methylated | PCa\|NT DMRs |
| chr16 | 88985176 | 88985946 | 0.000104349 | −24.19217687 | Hypo-methylated | PCa\|NT DMRs |
| chr16 | 88993501 | 88993646 | 0.000215808 | −21.11111111 | Hypo-methylated | PCa\|NT DMRs |
| chr16 | 89000311 | 89006583 | 4.55E−07 | 20.3780506 | Hyper-methylated | PCa\|NT DMRs |
| chr16 | 89023380 | 89025097 | 4.73E−09 | 32.69277504 | Hyper-methylated | PCa\|NT DMRs |
| chr16 | 89033864 | 89034223 | 8.31E−07 | 32.86375661 | Hyper-methylated | PCa\|NT DMRs |
| chr16 | 89035964 | 89037272 | 7.95E−05 | −26.18205128 | Hypo-methylated | PCa\|NT DMRs |
| chr16 | 89038036 | 89038197 | 0.000377317 | −23.21428571 | Hypo-methylated | PCa\|NT DMRs |
| chr16 | 89041832 | 89045234 | 1.34E−29 | 39.50926407 | Hyper-methylated | PCa\|NT DMRs |
| chr16 | 89047616 | 89047815 | 0.001969641 | −23.27222222 | Hypo-methylated | PCa\|NT DMRs |
| chr16 | 89050243 | 89050546 | 2.81E−09 | 29.98766234 | Hyper-methylated | PCa\|NT DMRs |
| chr16 | 89050732 | 89052291 | 6.69E−05 | −35.39761905 | Hypo-methylated | PCa\|NT DMRs |
| chr16 | 89071807 | 89080282 | 3.81E−05 | −27.62510823 | Hypo-methylated | PCa\|NT DMRs |
| chr16 | 89117560 | 89120260 | 8.84E−05 | −23.91227106 | Hypo-methylated | PCa\|NT DMRs |
| chr16 | 89129561 | 89155984 | 2.41E−18 | −20.75277986 | Hypo-methylated | PCa\|NT DMRs |
| chr16 | 89220176 | 89221053 | 0.000443725 | −38.65277778 | Hypo-methylated | PCa\|NT DMRs |
| chr16 | 89233831 | 89236933 | 0.000221737 | −26.1637037 | Hypo-methylated | PCa\|NT DMRs |
| chr16 | 89246570 | 89252105 | 8.37E−05 | −20.38095238 | Hypo-methylated | PCa\|NT DMRs |
| chr16 | 89261075 | 89261684 | 0.002960638 | 34.60767196 | Hyper-methylated | PCa\|NT DMRs |
| chr16 | 89308450 | 89313465 | 0.004656267 | −22.43760263 | Hypo-methylated | PCa\|NT DMRs |
| chr16 | 89380887 | 89380946 | 0.001969641 | −35.10714286 | Hypo-methylated | PCa\|NT DMRs |
| chr16 | 89410450 | 89411755 | 0.003544378 | −31.28984127 | Hypo-methylated | PCa\|NT DMRs |
| chr16 | 89473080 | 89473324 | 0.00064571 | 20.91156463 | Hyper-methylated | PCa\|NT DMRs |
| chr16 | 89497164 | 89497628 | 0.000974795 | −61.17916667 | Hypo-methylated | PCa\|NT DMRs |
| chr16 | 89632402 | 89632762 | 1.11E−06 | 21.56666667 | Hyper-methylated | PCa\|NT DMRs |
| chr16 | 89668771 | 89669085 | 0.000480383 | 30.12962963 | Hyper-methylated | PCa\|NT DMRs |
| chr16 | 89674813 | 89675952 | 0.000972067 | −48.75 | Hypo-methylated | PCa\|NT DMRs |
| chr16 | 89699579 | 89699790 | 0.001969641 | −40.35714286 | Hypo-methylated | PCa\|NT DMRs |
| chr16 | 89771520 | 89772073 | 1.69E−06 | −50.83193277 | Hypo-methylated | PCa\|NT DMRs |
| chr16 | 89909614 | 89909941 | 0.005968871 | 28.54112554 | Hyper-methylated | PCa\|NT DMRs |
| chr16 | 89919706 | 89920026 | 0.000205174 | −44.175 | Hypo-methylated | PCa\|NT DMRs |
| chr16 | 90075183 | 90077803 | 6.48E−05 | 42.73333333 | Hyper-methylated | PCa\|NT DMRs |
| chr17 | 29849 | 30033 | 0.006694433 | 24.52857143 | Hyper-methylated | PCa\|NT DMRs |
| chr17 | 142406 | 143470 | 0.000981741 | 20.30933333 | Hyper-methylated | PCa\|NT DMRs |
| chr17 | 154408 | 154672 | 0.005968871 | 37.3647619 | Hyper-methylated | PCa\|NT DMRs |
| chr17 | 234709 | 235109 | 0.000130897 | 26.87844612 | Hyper-methylated | PCa\|NT DMRs |
| chr17 | 263170 | 266675 | 1.42E−05 | 52.42823129 | Hyper-methylated | PCa\|NT DMRs |
| chr17 | 398090 | 398330 | 0.00064571 | 46.91496599 | Hyper-methylated | PCa\|NT DMRs |
| chr17 | 435760 | 440964 | 0.000406205 | 20.20607553 | Hyper-methylated | PCa\|NT DMRs |
| chr17 | 500943 | 505389 | 0.005968871 | 45.98857143 | Hyper-methylated | PCa\|NT DMRs |
| chr17 | 643211 | 643752 | 0.00179283 | 53.83333333 | Hyper-methylated | PCa\|NT DMRs |
| chr17 | 695027 | 695096 | 0.000205174 | 40.58035714 | Hyper-methylated | PCa\|NT DMRs |
| chr17 | 782822 | 783752 | 0.003544378 | −27.95396825 | Hypo-methylated | PCa\|NT DMRs |
| chr17 | 789739 | 790736 | 0.003314163 | −35.58857143 | Hypo-methylated | PCa\|NT DMRs |
| chr17 | 904928 | 911213 | 0.000193691 | −35.44662698 | Hypo-methylated | PCa\|NT DMRs |
| chr17 | 958978 | 960395 | 0.00071886 | −31.58004535 | Hypo-methylated | PCa\|NT DMRs |
| chr17 | 990491 | 991611 | 2.78E−15 | −47.48469388 | Hypo-methylated | PCa\|NT DMRs |
| chr17 | 1102425 | 1104766 | 1.34E−05 | 37.52585034 | Hyper-methylated | PCa\|NT DMRs |
| chr17 | 1131312 | 1132226 | 5.43E−11 | 27.34981685 | Hyper-methylated | PCa\|NT DMRs |
| chr17 | 1160033 | 1160311 | 1.53E−06 | 42.40340136 | Hyper-methylated | PCa\|NT DMRs |
| chr17 | 1191548 | 1193110 | 0.00064571 | −22.42857143 | Hypo-methylated | PCa\|NT DMRs |
| chr17 | 1251121 | 1272736 | 6.50E−06 | −25.31666667 | Hypo-methylated | PCa\|NT DMRs |
| chr17 | 1364119 | 1364319 | 0.004095673 | −31.67063492 | Hypo-methylated | PCa\|NT DMRs |
| chr17 | 1365444 | 1371366 | 8.31E−05 | 30.85769231 | Hyper-methylated | PCa\|NT DMRs |
| chr17 | 1372480 | 1372730 | 0.000145366 | −31.00612245 | Hypo-methylated | PCa\|NT DMRs |
| chr17 | 1372751 | 1372892 | 0.00064571 | 27.76734694 | Hyper-methylated | PCa\|NT DMRs |
| chr17 | 1373474 | 1373944 | 0.000138104 | 31.06776557 | Hyper-methylated | PCa\|NT DMRs |
| chr17 | 1389602 | 1390934 | 5.71E−10 | 31.2195122 | Hyper-methylated | PCa\|NT DMRs |
| chr17 | 1420921 | 1420970 | 0.001969641 | −38.55555556 | Hypo-methylated | PCa\|NT DMRs |
| chr17 | 1462651 | 1465163 | 0.002885118 | −30.85833333 | Hypo-methylated | PCa\|NT DMRs |
| chr17 | 1492163 | 1492298 | 6.98E−05 | 20.21598639 | Hyper-methylated | PCa\|NT DMRs |
| chr17 | 1507781 | 1508690 | 0.000173442 | 33.58670635 | Hyper-methylated | PCa\|NT DMRs |
| chr17 | 1518236 | 1521487 | 1.30E−05 | 21.29809524 | Hyper-methylated | PCa\|NT DMRs |
| chr17 | 1543014 | 1546412 | 2.71E−06 | −52.86375661 | Hypo-methylated | PCa\|NT DMRs |
| chr17 | 1547290 | 1549247 | 0.00109255 | 40.38660714 | Hyper-methylated | PCa\|NT DMRs |
| chr17 | 1650284 | 1650961 | 0.001610287 | −26.32202381 | Hypo-methylated | PCa\|NT DMRs |
| chr17 | 1657530 | 1657747 | 0.007959104 | 27.39795918 | Hyper-methylated | PCa\|NT DMRs |
| chr17 | 1824595 | 1824636 | 0.004095673 | −25.81904762 | Hypo-methylated | PCa\|NT DMRs |
| chr17 | 1843223 | 1843491 | 0.001985874 | −20.23142857 | Hypo-methylated | PCa\|NT DMRs |
| chr17 | 1880981 | 1881520 | 6.42E−10 | 29.71010453 | Hyper-methylated | PCa\|NT DMRs |
| chr17 | 1903143 | 1903487 | 0.000972067 | −26.91071429 | Hypo-methylated | PCa\|NT DMRs |
| chr17 | 1939737 | 1939853 | 0.005968871 | −51.67428571 | Hypo-methylated | PCa\|NT DMRs |
| chr17 | 2696048 | 2696222 | 0.000783791 | 39.47857143 | Hyper-methylated | PCa\|NT DMRs |
| chr17 | 2699505 | 2700793 | 7.69E−07 | 48.27238095 | Hyper-methylated | PCa\|NT DMRs |
| chr17 | 2755548 | 2756518 | 8.66E−09 | 36.44193548 | Hyper-methylated | PCa\|NT DMRs |
| chr17 | 2838934 | 2839897 | 2.44E−10 | 49.76551227 | Hyper-methylated | PCa\|NT DMRs |
| chr17 | 2866438 | 2881805 | 3.20E−05 | −35.44393241 | Hypo-methylated | PCa\|NT DMRs |
| chr17 | 2898695 | 2898871 | 0.000205174 | −63.85714286 | Hypo-methylated | PCa\|NT DMRs |
| chr17 | 3634937 | 3636151 | 0.003218673 | −20.11538462 | Hypo-methylated | PCa\|NT DMRs |
| chr17 | 3817683 | 3820741 | 6.72E−11 | 48.62039072 | Hyper-methylated | PCa\|NT DMRs |
| chr17 | 3867092 | 3867443 | 5.77E−09 | 26.68571429 | Hyper-methylated | PCa\|NT DMRs |
| chr17 | 3879911 | 3880230 | 0.001115302 | −36.30059524 | Hypo-methylated | PCa\|NT DMRs |
| chr17 | 3922850 | 3946272 | 0.008812478 | −20.16772487 | Hypo-methylated | PCa\|NT DMRs |
| chr17 | 4080331 | 4082167 | 0.002248181 | 29.47012987 | Hyper-methylated | PCa\|NT DMRs |
| chr17 | 4219263 | 4230877 | 0.007959104 | −41.54761905 | Hypo-methylated | PCa\|NT DMRs |
| chr17 | 4353144 | 4353352 | 0.001583613 | 29.00779221 | Hyper-methylated | PCa\|NT DMRs |
| chr17 | 4437886 | 4439446 | 0.000203452 | −31.45204082 | Hypo-methylated | PCa\|NT DMRs |
| chr17 | 4443016 | 4444737 | 0.000293988 | 24.26165414 | Hyper-methylated | PCa\|NT DMRs |
| chr17 | 4462702 | 4469956 | 2.64E−05 | −21.85407066 | Hypo-methylated | PCa\|NT DMRs |
| chr17 | 4498542 | 4498577 | 0.004095673 | 49.15 | Hyper-methylated | PCa\|NT DMRs |
| chr17 | 4553446 | 4553515 | 0.005968871 | 41.45714286 | Hyper-methylated | PCa\|NT DMRs |
| chr17 | 4641834 | 4642120 | 0.000305995 | 32.10714286 | Hyper-methylated | PCa\|NT DMRs |
| chr17 | 4711284 | 4711782 | 0.001115302 | −31.99107143 | Hypo-methylated | PCa\|NT DMRs |
| chr17 | 4831092 | 4835668 | 0.002225983 | 30.09714286 | Hyper-methylated | PCa\|NT DMRs |
| chr17 | 4973685 | 4977345 | 4.96E−06 | −32.11721612 | Hypo-methylated | PCa\|NT DMRs |
| chr17 | 5019150 | 5020004 | 4.33E−05 | 22.21396825 | Hyper-methylated | PCa\|NT DMRs |
| chr17 | 5402883 | 5403687 | 8.36E−06 | −25.18740399 | Hypo-methylated | PCa\|NT DMRs |
| chr17 | 5405260 | 5496850 | 2.00E−12 | −26.91718427 | Hypo-methylated | PCa\|NT DMRs |
| chr17 | 5516080 | 5609439 | 1.42E−09 | −27.38534799 | Hypo-methylated | PCa\|NT DMRs |
| chr17 | 5635333 | 5670876 | 2.05E−05 | −40.97835498 | Hypo-methylated | PCa\|NT DMRs |
| chr17 | 5689857 | 5703014 | 0.003314163 | −28.87142857 | Hypo-methylated | PCa\|NT DMRs |
| chr17 | 5846533 | 5973445 | 7.01E−09 | −25.21412429 | Hypo-methylated | PCa\|NT DMRs |
| chr17 | 6001587 | 6001955 | 0.001969641 | −37.71428571 | Hypo-methylated | PCa\|NT DMRs |
| chr17 | 6039466 | 6056920 | 0.002534928 | −29.98 | Hypo-methylated | PCa\|NT DMRs |
| chr17 | 6064415 | 6136117 | 3.99E−11 | −28.93428571 | Hypo-methylated | PCa\|NT DMRs |
| chr17 | 6155527 | 6297874 | 4.55E−15 | −31.96263736 | Hypo-methylated | PCa\|NT DMRs |
| chr17 | 6338195 | 6339581 | 0.003893879 | −28.28741497 | Hypo-methylated | PCa\|NT DMRs |
| chr17 | 6558851 | 6563630 | 2.06E−05 | 25.41879699 | Hyper-methylated | PCa\|NT DMRs |
| chr17 | 6898934 | 6899866 | 1.77E−17 | 30.5776455 | Hyper-methylated | PCa\|NT DMRs |
| chr17 | 6945391 | 6946780 | 2.27E−12 | 27.47997391 | Hyper-methylated | PCa\|NT DMRs |
| chr17 | 7004198 | 7004225 | 6.42E−05 | −36.60634921 | Hypo-methylated | PCa\|NT DMRs |
| chr17 | 7005914 | 7006210 | 0.00017871 | −22.40142857 | Hypo-methylated | PCa\|NT DMRs |
| chr17 | 7018443 | 7024314 | 0.00064571 | 41.08503401 | Hyper-methylated | PCa\|NT DMRs |
| chr17 | 7077404 | 7081510 | 4.81E−06 | 32.91278195 | Hyper-methylated | PCa\|NT DMRs |
| chr17 | 7082148 | 7093759 | 0.001399621 | −24.11688312 | Hypo-methylated | PCa\|NT DMRs |
| chr17 | 7111321 | 7112269 | 0.000418479 | −20.82633053 | Hypo-methylated | PCa\|NT DMRs |
| chr17 | 7114953 | 7116883 | 0.00068166 | −25.17440476 | Hypo-methylated | PCa\|NT DMRs |
| chr17 | 7165624 | 7165658 | 0.001241245 | −20.47619048 | Hypo-methylated | PCa\|NT DMRs |
| chr17 | 7185382 | 7185630 | 1.12E−07 | 21.75510204 | Hyper-methylated | PCa\|NT DMRs |
| chr17 | 7200469 | 7200737 | 0.000570576 | −28.07671958 | Hypo-methylated | PCa\|NT DMRs |
| chr17 | 7240013 | 7240106 | 0.00064571 | 39.97959184 | Hyper-methylated | PCa\|NT DMRs |
| chr17 | 7308911 | 7312016 | 5.51E−08 | 46.04395604 | Hyper-methylated | PCa\|NT DMRs |
| chr17 | 7321689 | 7330192 | 0.000563378 | −21.46992481 | Hypo-methylated | PCa\|NT DMRs |
| chr17 | 7344027 | 7348666 | 1.64E−06 | 20.93660714 | Hyper-methylated | PCa\|NT DMRs |
| chr17 | 7761950 | 7762833 | 6.69E−05 | −29.42142857 | Hypo-methylated | PCa\|NT DMRs |
| chr17 | 7806216 | 7807212 | 0.005968871 | −41.48917749 | Hypo-methylated | PCa\|NT DMRs |
| chr17 | 7814173 | 7814877 | 3.11E−06 | 30.34880952 | Hyper-methylated | PCa\|NT DMRs |
| chr17 | 8123516 | 8123706 | 0.005968871 | −51.47142857 | Hypo-methylated | PCa\|NT DMRs |
| chr17 | 8229868 | 8230459 | 2.57E−11 | 33.71688312 | Hyper-methylated | PCa\|NT DMRs |
| chr17 | 8311890 | 8311929 | 0.001969641 | 36.34920635 | Hyper-methylated | PCa\|NT DMRs |
| chr17 | 9011726 | 9011774 | 0.005968871 | 43.19428571 | Hyper-methylated | PCa\|NT DMRs |
| chr17 | 9018966 | 9019376 | 7.19E−09 | 22.21980952 | Hyper-methylated | PCa\|NT DMRs |
| chr17 | 9066153 | 9066358 | 1.37E−09 | −30.90299824 | Hypo-methylated | PCa\|NT DMRs |
| chr17 | 9079154 | 9081870 | 5.20E−05 | −24.57619048 | Hypo-methylated | PCa\|NT DMRs |
| chr17 | 9135926 | 9143838 | 5.10E−10 | 21.60593286 | Hyper-methylated | PCa\|NT DMRs |
| chr17 | 9144801 | 9144941 | 0.000974795 | 38.21428571 | Hyper-methylated | PCa\|NT DMRs |
| chr17 | 12856604 | 12877651 | 1.01E−06 | 24.37722944 | Hyper-methylated | PCa\|NT DMRs |
| chr17 | 12946737 | 12947377 | 0.006694433 | −25.65761905 | Hypo-methylated | PCa\|NT DMRs |
| chr17 | 13227831 | 13245284 | 0.005968871 | −35.7 | Hypo-methylated | PCa\|NT DMRs |
| chr17 | 13365965 | 13427776 | 1.34E−05 | −25.81394558 | Hypo-methylated | PCa\|NT DMRs |
| chr17 | 13503848 | 13504034 | 0.006589535 | 22.95714286 | Hyper-methylated | PCa\|NT DMRs |
| chr17 | 13553643 | 13557290 | 0.00068166 | −28.78869048 | Hypo-methylated | PCa\|NT DMRs |
| chr17 | 13950151 | 13950458 | 0.001969641 | −47.54761905 | Hypo-methylated | PCa\|NT DMRs |
| chr17 | 14059635 | 14115563 | 0.001050594 | 23.22786596 | Hyper-methylated | PCa\|NT DMRs |
| chr17 | 14207193 | 14207539 | 1.37E−06 | 24.99614512 | Hyper-methylated | PCa\|NT DMRs |
| chr17 | 14975063 | 15015703 | 5.99E−05 | −25.63589744 | Hypo-methylated | PCa\|NT DMRs |
| chr17 | 15849053 | 15849444 | 2.33E−10 | 22.32574405 | Hyper-methylated | PCa\|NT DMRs |
| chr17 | 16117366 | 16117689 | 0.001969641 | −27.53174603 | Hypo-methylated | PCa\|NT DMRs |
| chr17 | 16282382 | 16282455 | 0.005968871 | −32.17142857 | Hypo-methylated | PCa\|NT DMRs |
| chr17 | 16318945 | 16319187 | 0.00037547 | 26.09142857 | Hyper-methylated | PCa\|NT DMRs |
| chr17 | 16323248 | 16323482 | 0.001969641 | 44.02857143 | Hyper-methylated | PCa\|NT DMRs |
| chr17 | 16650051 | 16650093 | 0.00064571 | 24.14285714 | Hyper-methylated | PCa\|NT DMRs |
| chr17 | 16921326 | 16921487 | 0.005968871 | 21.6 | Hyper-methylated | PCa\|NT DMRs |
| chr17 | 17110285 | 17110574 | 0.001896858 | −29 | Hypo-methylated | PCa\|NT DMRs |
| chr17 | 17204363 | 17204425 | 0.001969641 | 40.02380952 | Hyper-methylated | PCa\|NT DMRs |
| chr17 | 17429414 | 17429982 | 2.05E−05 | −34.01082251 | Hypo-methylated | PCa\|NT DMRs |
| chr17 | 17460868 | 17461169 | 0.001181579 | −36.87755102 | Hypo-methylated | PCa\|NT DMRs |
| chr17 | 17603437 | 17604103 | 1.66E−15 | 23.78532397 | Hyper-methylated | PCa\|NT DMRs |
| chr17 | 17730932 | 17736460 | 0.002094 | −21.56150794 | Hypo-methylated | PCa\|NT DMRs |
| chr17 | 18885080 | 18885329 | 0.001115302 | −34.98214286 | Hypo-methylated | PCa\|NT DMRs |
| chr17 | 18906786 | 18907708 | 2.88E−05 | 21.82849336 | Hyper-methylated | PCa\|NT DMRs |
| chr17 | 18908404 | 18908427 | 0.00064571 | −24.67346939 | Hypo-methylated | PCa\|NT DMRs |
| chr17 | 19651508 | 19652057 | 0.000632596 | −27.86493506 | Hypo-methylated | PCa\|NT DMRs |
| chr17 | 19911626 | 19911791 | 0.001181579 | −25.7755102 | Hypo-methylated | PCa\|NT DMRs |
| chr17 | 20902570 | 20913160 | 0.000251555 | −27.25442177 | Hypo-methylated | PCa\|NT DMRs |
| chr17 | 21366806 | 21368047 | 2.65E−08 | 22.3837535 | Hyper-methylated | PCa\|NT DMRs |
| chr17 | 25676398 | 25676730 | 0.001458272 | 22.13897243 | Hyper-methylated | PCa\|NT DMRs |
| chr17 | 25793824 | 25793921 | 0.001969641 | 55.36269841 | Hyper-methylated | PCa\|NT DMRs |
| chr17 | 25914640 | 25915833 | 1.57E−07 | −25.08349206 | Hypo-methylated | PCa\|NT DMRs |
| chr17 | 26082735 | 26083043 | 0.00064571 | −36.59857143 | Hypo-methylated | PCa\|NT DMRs |
| chr17 | 26173143 | 26173408 | 0.004095673 | 52.90952381 | Hyper-methylated | PCa\|NT DMRs |
| chr17 | 26554245 | 26554488 | 0.007167999 | 22.34047619 | Hyper-methylated | PCa\|NT DMRs |
| chr17 | 26557402 | 26557584 | 0.001969641 | 29.61904762 | Hyper-methylated | PCa\|NT DMRs |
| chr17 | 26634050 | 26634336 | 7.47E−06 | 25.02891156 | Hyper-methylated | PCa\|NT DMRs |
| chr17 | 26794808 | 26795294 | 2.86E−06 | 37.9031746 | Hyper-methylated | PCa\|NT DMRs |
| chr17 | 27168687 | 27168829 | 0.001969641 | −43.75396825 | Hypo-methylated | PCa\|NT DMRs |
| chr17 | 27230875 | 27248052 | 0.000585936 | −24.75071429 | Hypo-methylated | PCa\|NT DMRs |
| chr17 | 27451976 | 27452039 | 0.005968871 | −28.05714286 | Hypo-methylated | PCa\|NT DMRs |
| chr17 | 27943248 | 27943897 | 1.76E−06 | 36.94047619 | Hyper-methylated | PCa\|NT DMRs |
| chr17 | 28562435 | 28562660 | 4.59E−07 | 31.01680672 | Hyper-methylated | PCa\|NT DMRs |
| chr17 | 29719101 | 29726026 | 3.06E−07 | 24.82324263 | Hyper-methylated | PCa\|NT DMRs |
| chr17 | 30023371 | 30023459 | 0.001969641 | −57.52380952 | Hypo-methylated | PCa\|NT DMRs |
| chr17 | 30089774 | 30090193 | 0.001181579 | −32.04081633 | Hypo-methylated | PCa\|NT DMRs |
| chr17 | 30215549 | 30215813 | 0.005968871 | 33.46666667 | Hyper-methylated | PCa\|NT DMRs |
| chr17 | 30243737 | 30243829 | 0.005968871 | −28.72095238 | Hypo-methylated | PCa\|NT DMRs |
| chr17 | 30676411 | 30676492 | 0.001969641 | −31.18253968 | Hypo-methylated | PCa\|NT DMRs |
| chr17 | 30847262 | 30847348 | 0.001969641 | 46.38095238 | Hyper-methylated | PCa\|NT DMRs |
| chr17 | 31649097 | 31776835 | 0.00071886 | −25.8462585 | Hypo-methylated | PCa\|NT DMRs |
| chr17 | 31806888 | 31825716 | 6.42E−05 | −26.48148148 | Hypo-methylated | PCa\|NT DMRs |
| chr17 | 32078874 | 32482283 | 2.39E−11 | −27.14239418 | Hypo-methylated | PCa\|NT DMRs |
| chr17 | 32482818 | 32483431 | 5.19E−07 | −32.37179487 | Hypo-methylated | PCa\|NT DMRs |
| chr17 | 32489947 | 32510198 | 0.007560755 | −36.41071429 | Hypo-methylated | PCa\|NT DMRs |
| chr17 | 32964325 | 32965263 | 2.17E−05 | 26.6447619 | Hyper-methylated | PCa\|NT DMRs |
| chr17 | 32968855 | 32980623 | 0.006410012 | −23.03846154 | Hypo-methylated | PCa\|NT DMRs |
| chr17 | 33446290 | 33446344 | 0.005513661 | 36.61488095 | Hyper-methylated | PCa\|NT DMRs |
| chr17 | 33487618 | 33518536 | 0.000225685 | −28.10045351 | Hypo-methylated | PCa\|NT DMRs |
| chr17 | 33715844 | 33735419 | 0.001052963 | −29.72244898 | Hypo-methylated | PCa\|NT DMRs |
| chr17 | 33808126 | 33809792 | 0.00641359 | −29.27698413 | Hypo-methylated | PCa\|NT DMRs |
| chr17 | 34058895 | 34059374 | 0.001969641 | 24.99013605 | Hyper-methylated | PCa\|NT DMRs |
| chr17 | 34077253 | 34077401 | 0.00064571 | 27.71428571 | Hyper-methylated | PCa\|NT DMRs |
| chr17 | 34079855 | 34080266 | 0.007560755 | 29.38511905 | Hyper-methylated | PCa\|NT DMRs |
| chr17 | 34121869 | 34122078 | 4.32E−05 | 21.44727891 | Hyper-methylated | PCa\|NT DMRs |
| chr17 | 34130944 | 34131414 | 0.00068166 | −37.41071429 | Hypo-methylated | PCa\|NT DMRs |
| chr17 | 34824909 | 34837275 | 2.02E−05 | −29.16613757 | Hypo-methylated | PCa\|NT DMRs |
| chr17 | 35014166 | 35014902 | 1.33E−09 | 23.84123113 | Hyper-methylated | PCa\|NT DMRs |
| chr17 | 35084323 | 35085068 | 0.001969641 | 44.88650794 | Hyper-methylated | PCa\|NT DMRs |
| chr17 | 35290924 | 35292442 | 8.19E−21 | 23.18267196 | Hyper-methylated | PCa\|NT DMRs |
| chr17 | 35294171 | 35295321 | 5.35E−07 | 28.14609524 | Hyper-methylated | PCa\|NT DMRs |
| chr17 | 35423389 | 35424186 | 0.001254235 | 21.68058608 | Hyper-methylated | PCa\|NT DMRs |
| chr17 | 35671538 | 35673094 | 0.000205174 | −27.41964286 | Hypo-methylated | PCa\|NT DMRs |
| chr17 | 35850854 | 35851134 | 0.001181579 | 25.37755102 | Hyper-methylated | PCa\|NT DMRs |
| chr17 | 36184154 | 36193063 | 0.001210062 | 35.0085034 | Hyper-methylated | PCa\|NT DMRs |
| chr17 | 36584917 | 36585790 | 2.94E−08 | 29.93972789 | Hyper-methylated | PCa\|NT DMRs |
| chr17 | 36841923 | 36842035 | 0.000205174 | 23.16071429 | Hyper-methylated | PCa\|NT DMRs |
| chr17 | 36898609 | 36898818 | 0.005968871 | −36.5952381 | Hypo-methylated | PCa\|NT DMRs |
| chr17 | 37023588 | 37024783 | 0.00064571 | 32.31972789 | Hyper-methylated | PCa\|NT DMRs |
| chr17 | 37045573 | 37048967 | 0.000342782 | 20.93082707 | Hyper-methylated | PCa\|NT DMRs |
| chr17 | 37321375 | 37332007 | 1.76E−06 | 23.97035147 | Hyper-methylated | PCa\|NT DMRs |
| chr17 | 37369289 | 37378813 | 0.000104435 | −30.1276644 | Hypo-methylated | PCa\|NT DMRs |
| chr17 | 37719830 | 37719914 | 0.001969641 | 26.21428571 | Hyper-methylated | PCa\|NT DMRs |
| chr17 | 37764094 | 37764285 | 0.000124779 | 23.72619048 | Hyper-methylated | PCa\|NT DMRs |
| chr17 | 37817230 | 37821665 | 3.28E−05 | −24.11657509 | Hypo-methylated | PCa\|NT DMRs |
| chr17 | 37875774 | 37875847 | 0.005968871 | 27.74285714 | Hyper-methylated | PCa\|NT DMRs |
| chr17 | 38100307 | 38100812 | 0.004505524 | −22.08571429 | Hypo-methylated | PCa\|NT DMRs |
| chr17 | 38212952 | 38213451 | 0.001969641 | −25.09126984 | Hypo-methylated | PCa\|NT DMRs |
| chr17 | 38334187 | 38334644 | 1.22E−09 | 39.42815126 | Hyper-methylated | PCa\|NT DMRs |
| chr17 | 38473024 | 38473412 | 3.15E−07 | −46.87301587 | Hypo-methylated | PCa\|NT DMRs |
| chr17 | 38476024 | 38481227 | 0.001775064 | 31.88756614 | Hyper-methylated | PCa\|NT DMRs |
| chr17 | 38487023 | 38488769 | 0.000158634 | −25.99019608 | Hypo-methylated | PCa\|NT DMRs |
| chr17 | 38599056 | 38599621 | 0.005295761 | 20.53630952 | Hyper-methylated | PCa\|NT DMRs |
| chr17 | 38774790 | 38775442 | 0.004505524 | −34.15119048 | Hypo-methylated | PCa\|NT DMRs |
| chr17 | 38935824 | 38936051 | 4.33E−05 | 26.35384615 | Hyper-methylated | PCa\|NT DMRs |
| chr17 | 39551441 | 39557930 | 0.005968871 | −34.5447619 | Hypo-methylated | PCa\|NT DMRs |
| chr17 | 39780606 | 39782133 | 0.002111644 | 26.01648352 | Hyper-methylated | PCa\|NT DMRs |
| chr17 | 39969240 | 39969580 | 6.66E−05 | 22.35273369 | Hyper-methylated | PCa\|NT DMRs |
| chr17 | 40125571 | 40127709 | 0.000159287 | −20.6254902 | Hypo-methylated | PCa\|NT DMRs |
| chr17 | 40192161 | 40192483 | 2.02E−08 | 34.74537815 | Hyper-methylated | PCa\|NT DMRs |
| chr17 | 40221718 | 40224992 | 1.19E−05 | 24.09163059 | Hyper-methylated | PCa\|NT DMRs |
| chr17 | 40259593 | 40260567 | 0.000583352 | 25.35555556 | Hyper-methylated | PCa\|NT DMRs |
| chr17 | 40340825 | 40341505 | 0.000205174 | 35.62916667 | Hyper-methylated | PCa\|NT DMRs |
| chr17 | 40440758 | 40441521 | 2.38E−10 | 43.47685185 | Hyper-methylated | PCa\|NT DMRs |
| chr17 | 40463426 | 40465243 | 0.000285978 | 31.64380952 | Hyper-methylated | PCa\|NT DMRs |
| chr17 | 40572399 | 40574560 | 6.77E−05 | 32.33333333 | Hyper-methylated | PCa\|NT DMRs |
| chr17 | 40705975 | 40706061 | 0.001181579 | −34.97278912 | Hypo-methylated | PCa\|NT DMRs |
| chr17 | 40723447 | 40723697 | 0.00064571 | −51.3707483 | Hypo-methylated | PCa\|NT DMRs |
| chr17 | 40811703 | 40811990 | 0.006589535 | 20.53619048 | Hyper-methylated | PCa\|NT DMRs |
| chr17 | 40835778 | 40836801 | 0.002818478 | −20.49977324 | Hypo-methylated | PCa\|NT DMRs |
| chr17 | 40931996 | 40932445 | 0.000611234 | −22.08507937 | Hypo-methylated | PCa\|NT DMRs |
| chr17 | 41177265 | 41177495 | 3.19E−08 | 27.53690476 | Hyper-methylated | PCa\|NT DMRs |
| chr17 | 41742803 | 41742993 | 0.005968871 | 20.13333333 | Hyper-methylated | PCa\|NT DMRs |
| chr17 | 41770802 | 41779529 | 0.00068166 | −26.19464286 | Hypo-methylated | PCa\|NT DMRs |
| chr17 | 42030536 | 42030962 | 0.000219415 | 20.43452381 | Hyper-methylated | PCa\|NT DMRs |
| chr17 | 42246099 | 42248359 | 0.005425083 | −31.66666667 | Hypo-methylated | PCa\|NT DMRs |
| chr17 | 42336559 | 42336709 | 1.29E−05 | 41.11607143 | Hyper-methylated | PCa\|NT DMRs |
| chr17 | 42357220 | 42357518 | 0.009924186 | 28.36507937 | Hyper-methylated | PCa\|NT DMRs |
| chr17 | 42386058 | 42386515 | 1.66E−06 | 27.51177945 | Hyper-methylated | PCa\|NT DMRs |
| chr17 | 42439761 | 42439985 | 0.005513661 | 35.21785714 | Hyper-methylated | PCa\|NT DMRs |
| chr17 | 42452205 | 42453166 | 0.000218021 | 29.39550265 | Hyper-methylated | PCa\|NT DMRs |
| chr17 | 42636124 | 42641018 | 2.20E−08 | 27.76587302 | Hyper-methylated | PCa\|NT DMRs |
| chr17 | 42685781 | 42685943 | 0.001969641 | −54.67460317 | Hypo-methylated | PCa\|NT DMRs |
| chr17 | 42835777 | 42836026 | 0.00266722 | 23.12745098 | Hyper-methylated | PCa\|NT DMRs |
| chr17 | 42992709 | 43001467 | 0.002750151 | −24.35587302 | Hypo-methylated | PCa\|NT DMRs |
| chr17 | 43120934 | 43122968 | 0.002654087 | −38.57380952 | Hypo-methylated | PCa\|NT DMRs |
| chr17 | 43198258 | 43198760 | 4.90E−17 | 46.61335201 | Hyper-methylated | PCa\|NT DMRs |
| chr17 | 43204387 | 43204802 | 0.001241245 | 32.74550265 | Hyper-methylated | PCa\|NT DMRs |
| chr17 | 43221187 | 43221967 | 5.40E−09 | 31.13064713 | Hyper-methylated | PCa\|NT DMRs |
| chr17 | 43299633 | 43302819 | 0.000177981 | 26.74145658 | Hyper-methylated | PCa\|NT DMRs |
| chr17 | 43311645 | 43319868 | 3.13E−10 | −50.33217189 | Hypo-methylated | PCa\|NT DMRs |
| chr17 | 43448920 | 43449280 | 3.92E−14 | 27.49028183 | Hyper-methylated | PCa\|NT DMRs |
| chr17 | 43482264 | 43483327 | 0.005968871 | −29.01809524 | Hypo-methylated | PCa\|NT DMRs |
| chr17 | 43723749 | 43723920 | 0.001969641 | 44.78571429 | Hyper-methylated | PCa\|NT DMRs |
| chr17 | 43772124 | 43787766 | 0.004553481 | −23.95396825 | Hypo-methylated | PCa\|NT DMRs |
| chr17 | 43860702 | 43861126 | 3.46E−11 | 20.10909091 | Hyper-methylated | PCa\|NT DMRs |
| chr17 | 43901001 | 43901196 | 0.001969641 | 24.16190476 | Hyper-methylated | PCa\|NT DMRs |
| chr17 | 44845792 | 44848771 | 7.23E−08 | 26.02951334 | Hyper-methylated | PCa\|NT DMRs |
| chr17 | 44953929 | 44972072 | 0.000535196 | −26.82205514 | Hypo-methylated | PCa\|NT DMRs |
| chr17 | 45772719 | 45773560 | 1.08E−05 | 33.67477954 | Hyper-methylated | PCa\|NT DMRs |
| chr17 | 45778236 | 45781740 | 0.00622356 | 28.17559524 | Hyper-methylated | PCa\|NT DMRs |
| chr17 | 45785806 | 45786739 | 5.61E−12 | −21.93438538 | Hypo-methylated | PCa\|NT DMRs |
| chr17 | 45821388 | 45821894 | 0.000688325 | −25.17765568 | Hypo-methylated | PCa\|NT DMRs |
| chr17 | 45897102 | 45897179 | 0.008482405 | 29.6 | Hyper-methylated | PCa\|NT DMRs |
| chr17 | 45924164 | 45925265 | 4.01E−13 | 26.26634492 | Hyper-methylated | PCa\|NT DMRs |
| chr17 | 45928959 | 45929684 | 0.000271988 | 38.50634921 | Hyper-methylated | PCa\|NT DMRs |
| chr17 | 46027094 | 46035273 | 0.002437742 | −38.21693122 | Hypo-methylated | PCa\|NT DMRs |
| chr17 | 46128598 | 46132436 | 0.000118733 | −24.66666667 | Hypo-methylated | PCa\|NT DMRs |
| chr17 | 46645891 | 46646578 | 1.42E−05 | 33.83877551 | Hyper-methylated | PCa\|NT DMRs |
| chr17 | 46663394 | 46671501 | 1.31E−11 | 32.6890377 | Hyper-methylated | PCa\|NT DMRs |
| chr17 | 46673604 | 46675173 | 4.37E−06 | 24.5264652 | Hyper-methylated | PCa\|NT DMRs |
| chr17 | 46679338 | 46679594 | 0.005968871 | −31.59714286 | Hypo-methylated | PCa\|NT DMRs |
| chr17 | 46696993 | 46697584 | 0.000175653 | −23.62962963 | Hypo-methylated | PCa\|NT DMRs |
| chr17 | 46719265 | 46719516 | 6.98E−05 | 24.76394558 | Hyper-methylated | PCa\|NT DMRs |
| chr17 | 46723587 | 46723790 | 0.005685503 | 21.10952381 | Hyper-methylated | PCa\|NT DMRs |
| chr17 | 47060483 | 47066626 | 0.003093471 | −23.95428571 | Hypo-methylated | PCa\|NT DMRs |
| chr17 | 47287931 | 47288173 | 0.003448944 | 24.07755102 | Hyper-methylated | PCa\|NT DMRs |
| chr17 | 47515946 | 47518536 | 5.66E−05 | 27.71315193 | Hyper-methylated | PCa\|NT DMRs |
| chr17 | 47519662 | 47535178 | 0.003152456 | −22.63015873 | Hypo-methylated | PCa\|NT DMRs |
| chr17 | 47547201 | 47547768 | 0.002400984 | 20.32063492 | Hyper-methylated | PCa\|NT DMRs |
| chr17 | 47985029 | 47992711 | 3.64E−14 | 35.02892416 | Hyper-methylated | PCa\|NT DMRs |
| chr17 | 48046982 | 48049652 | 7.81E−06 | 23.03174603 | Hyper-methylated | PCa\|NT DMRs |
| chr17 | 48071824 | 48072086 | 1.46E−06 | 20.1702381 | Hyper-methylated | PCa\|NT DMRs |
| chr17 | 48222642 | 48226084 | 0.004266047 | 30.61515152 | Hyper-methylated | PCa\|NT DMRs |
| chr17 | 48422457 | 48422588 | 0.008482405 | −56.47142857 | Hypo-methylated | PCa\|NT DMRs |
| chr17 | 48545943 | 48546774 | 5.14E−13 | 24.86825397 | Hyper-methylated | PCa\|NT DMRs |
| chr17 | 48559485 | 48560818 | 9.85E−06 | −20.12637363 | Hypo-methylated | PCa\|NT DMRs |
| chr17 | 48586118 | 48586184 | 0.000974795 | 41.94642857 | Hyper-methylated | PCa\|NT DMRs |
| chr17 | 48627969 | 48628618 | 0.000766778 | −38.77819549 | Hypo-methylated | PCa\|NT DMRs |
| chr17 | 48639261 | 48639794 | 0.000106046 | 26.94047619 | Hyper-methylated | PCa\|NT DMRs |
| chr17 | 48712473 | 48715144 | 0.00018063 | 24.7552795 | Hyper-methylated | PCa\|NT DMRs |
| chr17 | 48762191 | 48777009 | 6.19E−07 | −33.7567619 | Hypo-methylated | PCa\|NT DMRs |
| chr17 | 48911577 | 48912509 | 1.34E−05 | −25.16666667 | Hypo-methylated | PCa\|NT DMRs |
| chr17 | 48999145 | 49007170 | 0.009503366 | 37.10714286 | Hyper-methylated | PCa\|NT DMRs |
| chr17 | 49449345 | 49539553 | 0.002330844 | −29.77210884 | Hypo-methylated | PCa\|NT DMRs |
| chr17 | 49968510 | 50024784 | 0.005513661 | −38.38911565 | Hypo-methylated | PCa\|NT DMRs |
| chr17 | 53908225 | 53972771 | 0.007679542 | 25.11111111 | Hyper-methylated | PCa\|NT DMRs |
| chr17 | 54755992 | 54756199 | 1.29E−05 | 34.9829932 | Hyper-methylated | PCa\|NT DMRs |
| chr17 | 54776653 | 54787992 | 8.64E−05 | −47.94315476 | Hypo-methylated | PCa\|NT DMRs |
| chr17 | 55184069 | 55207150 | 0.000443725 | −23.04104308 | Hypo-methylated | PCa\|NT DMRs |
| chr17 | 55609803 | 55615176 | 0.000783791 | 38.25904762 | Hyper-methylated | PCa\|NT DMRs |
| chr17 | 55740465 | 55740792 | 0.000715461 | −22.0047619 | Hypo-methylated | PCa\|NT DMRs |
| chr17 | 55801427 | 55801633 | 0.00068166 | 28.44642857 | Hyper-methylated | PCa\|NT DMRs |
| chr17 | 55820329 | 55822306 | 0.007959104 | 20.55170068 | Hyper-methylated | PCa\|NT DMRs |
| chr17 | 55917271 | 55924746 | 0.002400984 | −23.94412698 | Hypo-methylated | PCa\|NT DMRs |
| chr17 | 55968004 | 55968073 | 0.008482405 | −21.95238095 | Hypo-methylated | PCa\|NT DMRs |
| chr17 | 56220091 | 56233772 | 1.53E−05 | −20.96560847 | Hypo-methylated | PCa\|NT DMRs |
| chr17 | 56245816 | 56255755 | 0.001451132 | −24.1042328 | Hypo-methylated | PCa\|NT DMRs |
| chr17 | 56405141 | 56421930 | 1.63E−09 | 44.32843915 | Hyper-methylated | PCa\|NT DMRs |
| chr17 | 56591006 | 56591545 | 0.001969641 | 23.47619048 | Hyper-methylated | PCa\|NT DMRs |
| chr17 | 56709848 | 56710050 | 0.003400006 | −22.53061224 | Hypo-methylated | PCa\|NT DMRs |
| chr17 | 56744087 | 56744491 | 0.000858283 | −20.22896825 | Hypo-methylated | PCa\|NT DMRs |
| chr17 | 59473114 | 59475769 | 1.40E−34 | 33.47424812 | Hyper-methylated | PCa\|NT DMRs |
| chr17 | 59652327 | 59652568 | 0.000205174 | −25.16369048 | Hypo-methylated | PCa\|NT DMRs |
| chr17 | 60757293 | 60757347 | 0.001850915 | −30.87202381 | Hypo-methylated | PCa\|NT DMRs |
| chr17 | 60757992 | 60758147 | 1.42E−05 | −31.49013605 | Hypo-methylated | PCa\|NT DMRs |
| chr17 | 60881193 | 60885780 | 3.74E−06 | 31.92626263 | Hyper-methylated | PCa\|NT DMRs |
| chr17 | 61354673 | 61354750 | 0.004095673 | 25.88095238 | Hyper-methylated | PCa\|NT DMRs |
| chr17 | 61497518 | 61497953 | 0.005968871 | 49.06952381 | Hyper-methylated | PCa\|NT DMRs |
| chr17 | 61509620 | 61510041 | 0.000205174 | −49.6875 | Hypo-methylated | PCa\|NT DMRs |
| chr17 | 61553864 | 61554253 | 6.69E−14 | 35.46235828 | Hyper-methylated | PCa\|NT DMRs |
| chr17 | 61614986 | 61616032 | 8.40E−08 | 34.1952381 | Hyper-methylated | PCa\|NT DMRs |
| chr17 | 61773963 | 61776940 | 4.36E−11 | 31.10787879 | Hyper-methylated | PCa\|NT DMRs |
| chr17 | 61817252 | 61817355 | 0.000632596 | −51.70822511 | Hypo-methylated | PCa\|NT DMRs |
| chr17 | 62008831 | 62010012 | 5.92E−06 | 42.94593838 | Hyper-methylated | PCa\|NT DMRs |
| chr17 | 62038982 | 62039170 | 0.000133909 | 23.32142857 | Hyper-methylated | PCa\|NT DMRs |
| chr17 | 62065549 | 62066996 | 1.43E−07 | −26.43446115 | Hypo-methylated | PCa\|NT DMRs |
| chr17 | 62141224 | 62141401 | 0.006225823 | 24.32727273 | Hyper-methylated | PCa\|NT DMRs |
| chr17 | 62773640 | 62777838 | 1.53E−55 | 33.94868316 | Hyper-methylated | PCa\|NT DMRs |
| chr17 | 63434457 | 63438808 | 0.002585277 | 25.64880952 | Hyper-methylated | PCa\|NT DMRs |
| chr17 | 64671880 | 64672869 | 6.83E−06 | −57.02436975 | Hypo-methylated | PCa\|NT DMRs |
| chr17 | 64954408 | 64954523 | 0.003892841 | −52.76772487 | Hypo-methylated | PCa\|NT DMRs |
| chr17 | 65796053 | 65803210 | 0.001518408 | −24.77886905 | Hypo-methylated | PCa\|NT DMRs |
| chr17 | 66194534 | 66195112 | 2.31E−05 | 21.07032967 | Hyper-methylated | PCa\|NT DMRs |
| chr17 | 66283529 | 66286130 | 0.009049402 | 28.15428571 | Hyper-methylated | PCa\|NT DMRs |
| chr17 | 66308871 | 66309259 | 0.002437742 | 34.32804233 | Hyper-methylated | PCa\|NT DMRs |
| chr17 | 66872632 | 66876198 | 1.11E−05 | −27.03030303 | Hypo-methylated | PCa\|NT DMRs |
| chr17 | 69971349 | 69971640 | 0.00064571 | −26.58843537 | Hypo-methylated | PCa\|NT DMRs |
| chr17 | 70119459 | 70120379 | 1.45E−23 | 28.26400227 | Hyper-methylated | PCa\|NT DMRs |
| chr17 | 70376170 | 70384952 | 0.000121489 | 25.52619048 | Hyper-methylated | PCa\|NT DMRs |
| chr17 | 71190109 | 71197723 | 0.002424722 | −34.44077381 | Hypo-methylated | PCa\|NT DMRs |
| chr17 | 71201722 | 71201801 | 0.005968871 | −45.37142857 | Hypo-methylated | PCa\|NT DMRs |
| chr17 | 71286873 | 71288160 | 0.002097193 | 39.8244898 | Hyper-methylated | PCa\|NT DMRs |
| chr17 | 71395462 | 71398377 | 0.00358897 | 24.49015873 | Hyper-methylated | PCa\|NT DMRs |
| chr17 | 71622064 | 71627573 | 0.001336455 | 25.21428571 | Hyper-methylated | PCa\|NT DMRs |
| chr17 | 71641633 | 71642099 | 0.003034084 | −25.12414966 | Hypo-methylated | PCa\|NT DMRs |
| chr17 | 71760111 | 71773459 | 0.001589893 | −20.91309524 | Hypo-methylated | PCa\|NT DMRs |
| chr17 | 71948480 | 71949246 | 2.39E−16 | 21.18130252 | Hyper-methylated | PCa\|NT DMRs |
| chr17 | 72090277 | 72098713 | 0.002097193 | −27.19931973 | Hypo-methylated | PCa\|NT DMRs |
| chr17 | 72209865 | 72210375 | 2.22E−11 | 37.48730159 | Hyper-methylated | PCa\|NT DMRs |
| chr17 | 72284185 | 72301189 | 0.000377317 | −25.84047619 | Hypo-methylated | PCa\|NT DMRs |
| chr17 | 72343982 | 72346992 | 1.08E−06 | −23.11142857 | Hypo-methylated | PCa\|NT DMRs |
| chr17 | 72462522 | 72463272 | 4.11E−07 | 45.86466165 | Hyper-methylated | PCa\|NT DMRs |
| chr17 | 72464888 | 72476525 | 5.18E−06 | −20.04550265 | Hypo-methylated | PCa\|NT DMRs |
| chr17 | 72535504 | 72545983 | 4.89E−07 | −20.97047619 | Hypo-methylated | PCa\|NT DMRs |
| chr17 | 72645290 | 72657724 | 2.94E−06 | −29.88359788 | Hypo-methylated | PCa\|NT DMRs |
| chr17 | 72707221 | 72708885 | 0.001181579 | 40.71428571 | Hyper-methylated | PCa\|NT DMRs |
| chr17 | 72736981 | 72741459 | 0.002802605 | −26.41176471 | Hypo-methylated | PCa\|NT DMRs |
| chr17 | 72755638 | 72756260 | 0.005968871 | 20.88190476 | Hyper-methylated | PCa\|NT DMRs |
| chr17 | 72782308 | 72786474 | 1.50E−05 | 32.41704261 | Hyper-methylated | PCa\|NT DMRs |
| chr17 | 72848842 | 72849368 | 6.45E−05 | 21.94313725 | Hyper-methylated | PCa\|NT DMRs |
| chr17 | 72857995 | 72858033 | 0.007959104 | 20.69387755 | Hyper-methylated | PCa\|NT DMRs |
| chr17 | 72913566 | 72916435 | 3.90E−10 | 26.47706767 | Hyper-methylated | PCa\|NT DMRs |
| chr17 | 73200735 | 73201068 | 0.007560755 | −28.86796537 | Hypo-methylated | PCa\|NT DMRs |
| chr17 | 73321299 | 73323321 | 0.002585277 | 21.1875 | Hyper-methylated | PCa\|NT DMRs |
| chr17 | 73437189 | 73440280 | 0.005122815 | −31.19264069 | Hypo-methylated | PCa\|NT DMRs |
| chr17 | 73494058 | 73494362 | 0.000205174 | 41.96428571 | Hyper-methylated | PCa\|NT DMRs |
| chr17 | 73545489 | 73553691 | 7.34E−05 | −39.05555556 | Hypo-methylated | PCa\|NT DMRs |
| chr17 | 73717684 | 73717837 | 0.003871035 | −21.28373016 | Hypo-methylated | PCa\|NT DMRs |
| chr17 | 73824171 | 73824344 | 0.005968871 | 33.46666667 | Hyper-methylated | PCa\|NT DMRs |
| chr17 | 73838733 | 73841195 | 0.001245178 | 41.5202381 | Hyper-methylated | PCa\|NT DMRs |
| chr17 | 73992997 | 73993517 | 0.000974795 | 44.4672619 | Hyper-methylated | PCa\|NT DMRs |
| chr17 | 73996299 | 73997315 | 0.000122432 | −26.41788932 | Hypo-methylated | PCa\|NT DMRs |
| chr17 | 73999632 | 74001745 | 0.001181579 | −55.0755102 | Hypo-methylated | PCa\|NT DMRs |
| chr17 | 74032659 | 74033034 | 0.00064571 | −55.6244898 | Hypo-methylated | PCa\|NT DMRs |
| chr17 | 74128537 | 74128951 | 0.000286238 | −42.08937729 | Hypo-methylated | PCa\|NT DMRs |
| chr17 | 74136213 | 74136587 | 3.21E−14 | 32.79128482 | Hyper-methylated | PCa\|NT DMRs |
| chr17 | 74235859 | 74236109 | 2.04E−05 | 25.94642857 | Hyper-methylated | PCa\|NT DMRs |
| chr17 | 74267548 | 74268760 | 0.009214933 | 27.4789916 | Hyper-methylated | PCa\|NT DMRs |
| chr17 | 74275125 | 74275552 | 0.005968871 | −30.58791209 | Hypo-methylated | PCa\|NT DMRs |
| chr17 | 74377011 | 74378569 | 1.21E−07 | 48.6210084 | Hyper-methylated | PCa\|NT DMRs |
| chr17 | 74485765 | 74490064 | 0.001054317 | 39.36054422 | Hyper-methylated | PCa\|NT DMRs |
| chr17 | 74527294 | 74529730 | 0.004178506 | −32.06577381 | Hypo-methylated | PCa\|NT DMRs |
| chr17 | 74557626 | 74565009 | 0.000960149 | 29.15535714 | Hyper-methylated | PCa\|NT DMRs |
| chr17 | 74566041 | 74566431 | 0.003892841 | −36.43121693 | Hypo-methylated | PCa\|NT DMRs |
| chr17 | 74822659 | 74823027 | 2.86E−05 | 29.57142857 | Hyper-methylated | PCa\|NT DMRs |
| chr17 | 74994454 | 74994805 | 6.42E−05 | 47.8 | Hyper-methylated | PCa\|NT DMRs |
| chr17 | 75074776 | 75079364 | 0.007497996 | −20.92857143 | Hypo-methylated | PCa\|NT DMRs |
| chr17 | 75231604 | 75231816 | 0.000481535 | 53.50793651 | Hyper-methylated | PCa\|NT DMRs |
| chr17 | 75281936 | 75282041 | 0.001969641 | 40.90408163 | Hyper-methylated | PCa\|NT DMRs |
| chr17 | 75311663 | 75312391 | 0.001969641 | 48.9484127 | Hyper-methylated | PCa\|NT DMRs |
| chr17 | 75313789 | 75319024 | 0.003917805 | 24.04537815 | Hyper-methylated | PCa\|NT DMRs |
| chr17 | 75385167 | 75387251 | 7.59E−06 | 34.81994048 | Hyper-methylated | PCa\|NT DMRs |
| chr17 | 75421158 | 75422626 | 0.000128095 | 31.29980159 | Hyper-methylated | PCa\|NT DMRs |
| chr17 | 75448405 | 75450254 | 0.000431345 | 28.65297619 | Hyper-methylated | PCa\|NT DMRs |
| chr17 | 75450700 | 75462536 | 1.68E−12 | 32.7140625 | Hyper-methylated | PCa\|NT DMRs |
| chr17 | 75468686 | 75471491 | 0.00064571 | 35.1047619 | Hyper-methylated | PCa\|NT DMRs |
| chr17 | 75523255 | 75523479 | 0.001344447 | −36.55357143 | Hypo-methylated | PCa\|NT DMRs |
| chr17 | 75530879 | 75540041 | 5.46E−05 | −22.39047619 | Hypo-methylated | PCa\|NT DMRs |
| chr17 | 75567588 | 75568711 | 0.000364072 | 30.41208791 | Hyper-methylated | PCa\|NT DMRs |
| chr17 | 75652314 | 75674045 | 6.72E−09 | −24.59408369 | Hypo-methylated | PCa\|NT DMRs |
| chr17 | 75703130 | 75705285 | 3.39E−05 | −30.92770563 | Hypo-methylated | PCa\|NT DMRs |
| chr17 | 75751822 | 75759239 | 0.001181579 | −42.10204082 | Hypo-methylated | PCa\|NT DMRs |
| chr17 | 75805015 | 75808038 | 0.007560755 | −21.22261905 | Hypo-methylated | PCa\|NT DMRs |
| chr17 | 75818675 | 75820648 | 0.001709403 | −22.45416667 | Hypo-methylated | PCa\|NT DMRs |
| chr17 | 75827381 | 75827782 | 6.42E−05 | −23.52380952 | Hypo-methylated | PCa\|NT DMRs |
| chr17 | 75842522 | 75843624 | 0.002585277 | 32.10714286 | Hyper-methylated | PCa\|NT DMRs |
| chr17 | 75865566 | 75865990 | 0.005186056 | 28.64809524 | Hyper-methylated | PCa\|NT DMRs |
| chr17 | 76112056 | 76112134 | 0.005968871 | 40.4 | Hyper-methylated | PCa\|NT DMRs |
| chr17 | 76117002 | 76124173 | 4.26E−06 | 23.72311111 | Hyper-methylated | PCa\|NT DMRs |
| chr17 | 76126263 | 76133937 | 2.95E−17 | 30.1315735 | Hyper-methylated | PCa\|NT DMRs |
| chr17 | 76140951 | 76141577 | 0.000161233 | −40.37428571 | Hypo-methylated | PCa\|NT DMRs |
| chr17 | 76160493 | 76160549 | 0.005968871 | −24.57619048 | Hypo-methylated | PCa\|NT DMRs |
| chr17 | 76167495 | 76167900 | 0.000570576 | −22.52910053 | Hypo-methylated | PCa\|NT DMRs |
| chr17 | 76170950 | 76171881 | 3.80E−06 | −34.27113997 | Hypo-methylated | PCa\|NT DMRs |
| chr17 | 76172781 | 76172850 | 6.42E−05 | 35.3015873 | Hyper-methylated | PCa\|NT DMRs |
| chr17 | 76227803 | 76228830 | 1.24E−10 | 25.5248227 | Hyper-methylated | PCa\|NT DMRs |
| chr17 | 76361174 | 76361451 | 0.000149331 | 39.68831169 | Hyper-methylated | PCa\|NT DMRs |
| chr17 | 76361762 | 76362800 | 2.03E−06 | −22.60888889 | Hypo-methylated | PCa\|NT DMRs |
| chr17 | 76380937 | 76385429 | 0.00179283 | 35.45892857 | Hyper-methylated | PCa\|NT DMRs |
| chr17 | 76472324 | 76478296 | 3.20E−06 | −22.53501401 | Hypo-methylated | PCa\|NT DMRs |
| chr17 | 76479834 | 76481381 | 0.008083098 | 24.7047619 | Hyper-methylated | PCa\|NT DMRs |
| chr17 | 76586441 | 76596569 | 0.000149336 | −24.89411765 | Hypo-methylated | PCa\|NT DMRs |
| chr17 | 76646502 | 76654718 | 0.00023371 | −35.09215686 | Hypo-methylated | PCa\|NT DMRs |
| chr17 | 76661489 | 76662907 | 0.001181579 | 39.55102041 | Hyper-methylated | PCa\|NT DMRs |
| chr17 | 76712831 | 76713341 | 0.000305995 | −47.15666667 | Hypo-methylated | PCa\|NT DMRs |
| chr17 | 76886583 | 76887592 | 2.86E−05 | 49.75824176 | Hyper-methylated | PCa\|NT DMRs |
| chr17 | 76953441 | 76953488 | 0.005968871 | −52.56571429 | Hypo-methylated | PCa\|NT DMRs |
| chr17 | 76970989 | 76971731 | 6.16E−06 | −45.53869048 | Hypo-methylated | PCa\|NT DMRs |
| chr17 | 77021079 | 77021828 | 0.000112344 | −34.15396825 | Hypo-methylated | PCa\|NT DMRs |
| chr17 | 77092717 | 77092978 | 0.009924186 | −29.52698413 | Hypo-methylated | PCa\|NT DMRs |
| chr17 | 77099798 | 77100321 | 0.000243577 | 30.77113095 | Hyper-methylated | PCa\|NT DMRs |
| chr17 | 77110607 | 77111617 | 0.000149244 | −37.78630952 | Hypo-methylated | PCa\|NT DMRs |
| chr17 | 77121790 | 77123774 | 6.68E−05 | −25.98039216 | Hypo-methylated | PCa\|NT DMRs |
| chr17 | 77145368 | 77152976 | 1.47E−12 | −31.54484127 | Hypo-methylated | PCa\|NT DMRs |
| chr17 | 77175610 | 77177504 | 5.87E−08 | −25.47380952 | Hypo-methylated | PCa\|NT DMRs |
| chr17 | 77190768 | 77215871 | 7.72E−13 | −22.84778613 | Hypo-methylated | PCa\|NT DMRs |
| chr17 | 77285423 | 77293777 | 1.13E−05 | −28.46813187 | Hypo-methylated | PCa\|NT DMRs |
| chr17 | 77315523 | 77317949 | 0.000974795 | −33.95119048 | Hypo-methylated | PCa\|NT DMRs |
| chr17 | 77325839 | 77365381 | 9.56E−16 | −28.140764 | Hypo-methylated | PCa\|NT DMRs |
| chr17 | 77395158 | 77398979 | 0.00010532 | −22.84920635 | Hypo-methylated | PCa\|NT DMRs |
| chr17 | 77536253 | 77546171 | 8.77E−12 | −22.04505495 | Hypo-methylated | PCa\|NT DMRs |
| chr17 | 77660432 | 77660752 | 0.007428376 | 20.49450549 | Hyper-methylated | PCa\|NT DMRs |
| chr17 | 77674065 | 77674217 | 0.001969641 | 34.23809524 | Hyper-methylated | PCa\|NT DMRs |
| chr17 | 77722150 | 77722457 | 7.48E−11 | 22.51587302 | Hyper-methylated | PCa\|NT DMRs |
| chr17 | 77746456 | 77747566 | 0.001171269 | −27.64935065 | Hypo-methylated | PCa\|NT DMRs |
| chr17 | 77755583 | 77755966 | 0.000480383 | 54.88624339 | Hyper-methylated | PCa\|NT DMRs |
| chr17 | 77764781 | 77766130 | 1.40E−17 | 36.97878028 | Hyper-methylated | PCa\|NT DMRs |
| chr17 | 77788000 | 77788439 | 6.42E−05 | 34.53968254 | Hyper-methylated | PCa\|NT DMRs |
| chr17 | 77893070 | 77894006 | 2.75E−07 | −37.50272109 | Hypo-methylated | PCa\|NT DMRs |
| chr17 | 77894032 | 77895092 | 0.000145094 | 27.40079365 | Hyper-methylated | PCa\|NT DMRs |
| chr17 | 77898859 | 77899654 | 0.003448944 | −21.15391705 | Hypo-methylated | PCa\|NT DMRs |
| chr17 | 77909301 | 77911936 | 0.00064571 | −28.73015873 | Hypo-methylated | PCa\|NT DMRs |
| chr17 | 77922731 | 77923161 | 0.000187865 | −25.10277778 | Hypo-methylated | PCa\|NT DMRs |
| chr17 | 77960558 | 77960715 | 3.20E−06 | −46.69187675 | Hypo-methylated | PCa\|NT DMRs |
| chr17 | 77966181 | 77966505 | 0.006190613 | −20.97835498 | Hypo-methylated | PCa\|NT DMRs |
| chr17 | 77982180 | 77983542 | 1.17E−05 | −21.67728938 | Hypo-methylated | PCa\|NT DMRs |
| chr17 | 77996733 | 78009160 | 3.17E−07 | −24.01991342 | Hypo-methylated | PCa\|NT DMRs |
| chr17 | 78041377 | 78044790 | 3.26E−10 | −34.42528736 | Hypo-methylated | PCa\|NT DMRs |
| chr17 | 78161083 | 78161860 | 4.84E−06 | −44.61772487 | Hypo-methylated | PCa\|NT DMRs |
| chr17 | 78169924 | 78169998 | 0.007628253 | 29.18707483 | Hyper-methylated | PCa\|NT DMRs |
| chr17 | 78235448 | 78251123 | 0.006791526 | 23.41927438 | Hyper-methylated | PCa\|NT DMRs |
| chr17 | 78262477 | 78262609 | 0.001969641 | 23.63730159 | Hyper-methylated | PCa\|NT DMRs |
| chr17 | 78424123 | 78424297 | 0.001115302 | −29.16964286 | Hypo-methylated | PCa\|NT DMRs |
| chr17 | 78754323 | 78755215 | 0.000205174 | 50.53869048 | Hyper-methylated | PCa\|NT DMRs |
| chr17 | 78845066 | 78847753 | 3.79E−05 | 25.11287478 | Hyper-methylated | PCa\|NT DMRs |
| chr17 | 78853824 | 78854059 | 0.001789867 | 32.27195767 | Hyper-methylated | PCa\|NT DMRs |
| chr17 | 78860036 | 78860413 | 9.81E−07 | 25.53333333 | Hyper-methylated | PCa\|NT DMRs |
| chr17 | 78915627 | 78916403 | 0.000729529 | −34.82380952 | Hypo-methylated | PCa\|NT DMRs |
| chr17 | 78957505 | 78962207 | 0.001241245 | −21.7956044 | Hypo-methylated | PCa\|NT DMRs |
| chrl7 | 78963414 | 78965075 | 0.005968871 | −23.02579365 | Hypo-methylated | PCa\|NT DMRs |
| chr17 | 78974683 | 78975232 | 0.00080034 | −29.84017857 | Hypo-methylated | PCa\|NT DMRs |
| chr17 | 78999350 | 79001859 | 3.75E−06 | −26.14307359 | Hypo-methylated | PCa\|NT DMRs |
| chr17 | 79024835 | 79025692 | 0.000339627 | −23.70595238 | Hypo-methylated | PCa\|NT DMRs |
| chr17 | 79028749 | 79041678 | 7.45E−07 | −21.17080745 | Hypo-methylated | PCa\|NT DMRs |
| chr17 | 79042879 | 79043265 | 2.31E−05 | −34.92307692 | Hypo-methylated | PCa\|NT DMRs |
| chr17 | 79066746 | 79067196 | 6.29E−09 | −23.35185185 | Hypo-methylated | PCa\|NT DMRs |
| chr17 | 79095365 | 79098041 | 8.33E−28 | −47.30533752 | Hypo-methylated | PCa\|NT DMRs |
| chr17 | 79125228 | 79125324 | 0.001093287 | 28.08253968 | Hyper-methylated | PCa\|NT DMRs |
| chr17 | 79133573 | 79134646 | 5.31E−06 | 22.31492063 | Hyper-methylated | PCa\|NT DMRs |
| chr17 | 79138730 | 79139351 | 5.50E−05 | −21.84210526 | Hypo-methylated | PCa\|NT DMRs |
| chr17 | 79213998 | 79214135 | 0.000205174 | −27 | Hypo-methylated | PCa\|NT DMRs |
| chr17 | 79258585 | 79260095 | 5.15E−08 | 37.69627976 | Hyper-methylated | PCa\|NT DMRs |
| chr17 | 79261627 | 79262123 | 1.42E−05 | 44.06292517 | Hyper-methylated | PCa\|NT DMRs |
| chr17 | 79283832 | 79283926 | 0.000122432 | 24.35497835 | Hyper-methylated | PCa\|NT DMRs |
| chr17 | 79297386 | 79297773 | 0.000117699 | 45.94805195 | Hyper-methylated | PCa\|NT DMRs |
| chr17 | 79375341 | 79375809 | 2.86E−05 | 43.30769231 | Hyper-methylated | PCa\|NT DMRs |
| chr17 | 79395317 | 79396928 | 0.006976128 | 21.91764706 | Hyper-methylated | PCa\|NT DMRs |
| chr17 | 79404294 | 79409509 | 7.39E−08 | 23.66557272 | Hyper-methylated | PCa\|NT DMRs |
| chr17 | 79427225 | 79428894 | 1.27E−19 | 35.98879552 | Hyper-methylated | PCa\|NT DMRs |
| chr17 | 79450263 | 79451215 | 1.28E−05 | 28.67654987 | Hyper-methylated | PCa\|NT DMRs |
| chr17 | 79503591 | 79504469 | 2.51E−23 | 30.19090354 | Hyper-methylated | PCa\|NT DMRs |
| chr17 | 79619878 | 79620666 | 0.00032872 | 34.90884354 | Hyper-methylated | PCa\|NT DMRs |
| chr17 | 79626532 | 79626972 | 1.97E−05 | −53.22619048 | Hypo-methylated | PCa\|NT DMRs |
| chr17 | 79649567 | 79649845 | 0.002585277 | −28.875 | Hypo-methylated | PCa\|NT DMRs |
| chr17 | 79652015 | 79653971 | 2.56E−05 | −30.54444444 | Hypo-methylated | PCa\|NT DMRs |
| chr17 | 79668608 | 79668747 | 4.06E−05 | −23.95535714 | Hypo-methylated | PCa\|NT DMRs |
| chr17 | 79679794 | 79681312 | 0.002562638 | −39.30873016 | Hypo-methylated | PCa\|NT DMRs |
| chr17 | 79694463 | 79694557 | 0.003464153 | −44.50649351 | Hypo-methylated | PCa\|NT DMRs |
| chr17 | 79701435 | 79701791 | 3.14E−05 | −24.77142857 | Hypo-methylated | PCa\|NT DMRs |
| chr17 | 79707782 | 79708510 | 1.44E−05 | 28.48245614 | Hyper-methylated | PCa\|NT DMRs |
| chr17 | 79763216 | 79766197 | 0.001000355 | −22.28666667 | Hypo-methylated | PCa\|NT DMRs |
| chr17 | 79873535 | 79873817 | 0.000392715 | −22.6047619 | Hypo-methylated | PCa\|NT DMRs |
| chr17 | 79880614 | 79883766 | 4.43E−06 | 25.99514286 | Hyper-methylated | PCa\|NT DMRs |
| chr17 | 79887989 | 79888329 | 0.006410012 | −20.55367965 | Hypo-methylated | PCa\|NT DMRs |
| chr17 | 79897923 | 79898002 | 4.43E−05 | −29.19413919 | Hypo-methylated | PCa\|NT DMRs |
| chr17 | 79919556 | 79920603 | 5.05E−07 | 28.08444444 | Hyper-methylated | PCa\|NT DMRs |
| chr17 | 79949256 | 79950562 | 1.26E−06 | −35.66954596 | Hypo-methylated | PCa\|NT DMRs |
| chr17 | 79977130 | 79979628 | 5.04E−05 | −30.23619048 | Hypo-methylated | PCa\|NT DMRs |
| chr17 | 79991872 | 79992855 | 2.37E−05 | −35.01333333 | Hypo-methylated | PCa\|NT DMRs |
| chr17 | 80012549 | 80022967 | 3.24E−12 | −28.54686248 | Hypo-methylated | PCa\|NT DMRs |
| chr17 | 80030597 | 80054365 | 4.93E−116 | −35.97386377 | Hypo-methylated | PCa\|NT DMRs |
| chr17 | 80174767 | 80175804 | 7.84E−06 | 39.97714286 | Hyper-methylated | PCa\|NT DMRs |
| chr17 | 80185958 | 80186766 | 3.44E−12 | 38.51873706 | Hyper-methylated | PCa\|NT DMRs |
| chr17 | 80215659 | 80225606 | 0.008497149 | −24.8047619 | Hypo-methylated | PCa\|NT DMRs |
| chr17 | 80253604 | 80254273 | 0.00064571 | −38.41156463 | Hypo-methylated | PCa\|NT DMRs |
| chr17 | 80259204 | 80263421 | 5.89E−11 | 26.61152882 | Hyper-methylated | PCa\|NT DMRs |
| chr17 | 80267886 | 80268482 | 0.001344447 | −23.32142857 | Hypo-methylated | PCa\|NT DMRs |
| chr17 | 80292191 | 80292796 | 5.44E−05 | 28.75 | Hyper-methylated | PCa\|NT DMRs |
| chr17 | 80308407 | 80309325 | 0.00023371 | 24.25578231 | Hyper-methylated | PCa\|NT DMRs |
| chr17 | 80341259 | 80343302 | 1.44E−05 | −27.50822511 | Hypo-methylated | PCa\|NT DMRs |
| chr17 | 80345995 | 80346517 | 0.000339627 | −21.5077381 | Hypo-methylated | PCa\|NT DMRs |
| chr17 | 80357183 | 80358923 | 0.000117699 | 49.29437229 | Hyper-methylated | PCa\|NT DMRs |
| chr17 | 80593887 | 80597555 | 1.97E−05 | −51.6452381 | Hypo-methylated | PCa\|NT DMRs |
| chr17 | 80636580 | 80636678 | 0.000205174 | −33.88690476 | Hypo-methylated | PCa\|NT DMRs |
| chr17 | 80660649 | 80660846 | 0.001969641 | −51.43253968 | Hypo-methylated | PCa\|NT DMRs |
| chr17 | 80846867 | 80850475 | 0.000107028 | −23.90029762 | Hypo-methylated | PCa\|NT DMRs |
| chr17 | 80923382 | 80926233 | 0.002097193 | −38.26530612 | Hypo-methylated | PCa\|NT DMRs |
| chr17 | 80943851 | 80944094 | 0.000474737 | 47.2037037 | Hyper-methylated | PCa\|NT DMRs |
| chr17 | 80981902 | 80982435 | 0.000286238 | 33.8484127 | Hyper-methylated | PCa\|NT DMRs |
| chr17 | 81025841 | 81026757 | 6.69E−05 | −33.34 | Hypo-methylated | PCa\|NT DMRs |
| chr17 | 81026919 | 81027222 | 0.000117699 | 50.30735931 | Hyper-methylated | PCa\|NT DMRs |
| chr17 | 81047008 | 81047855 | 4.36E−12 | −48.49659864 | Hypo-methylated | PCa\|NT DMRs |
| chr17 | 81054469 | 81055049 | 3.20E−05 | −39.6292517 | Hypo-methylated | PCa\|NT DMRs |
| chr17 | 81057062 | 81057715 | 0.002285154 | 22.19761905 | Hyper-methylated | PCa\|NT DMRs |
| chr17 | 81071958 | 81072216 | 1.76E−06 | −28.04761905 | Hypo-methylated | PCa\|NT DMRs |
| chr17 | 81083398 | 81085505 | 1.93E−10 | −21.96056166 | Hypo-methylated | PCa\|NT DMRs |
| chr17 | 81143889 | 81148046 | 3.10E−08 | 37.56075534 | Hyper-methylated | PCa\|NT DMRs |
| chr18 | 465930 | 466038 | 0.001969641 | 22.07142857 | Hyper-methylated | PCa\|NT DMRs |
| chr18 | 501047 | 501653 | 0.009924186 | 22.09899749 | Hyper-methylated | PCa\|NT DMRs |
| chr18 | 514363 | 514574 | 0.007560755 | −21.24155844 | Hypo-methylated | PCa\|NT DMRs |
| chr18 | 904493 | 905101 | 8.31E−07 | 21.65538847 | Hyper-methylated | PCa\|NT DMRs |
| chr18 | 951739 | 951983 | 0.005968871 | −37.13333333 | Hypo-methylated | PCa\|NT DMRs |
| chr18 | 1676045 | 1955468 | 1.01E−14 | −36.68706004 | Hypo-methylated | PCa\|NT DMRs |
| chr18 | 2033276 | 2192354 | 2.01E−07 | −39.28928571 | Hypo-methylated | PCa\|NT DMRs |
| chr18 | 2270060 | 2471687 | 1.35E−11 | −30.58783069 | Hypo-methylated | PCa\|NT DMRs |
| chr18 | 2858012 | 2859081 | 0.006410012 | 35.35654762 | Hyper-methylated | PCa\|NT DMRs |
| chr18 | 3411918 | 3412165 | 0.000149006 | 29.23203463 | Hyper-methylated | PCa\|NT DMRs |
| chr18 | 3499025 | 3499861 | 1.46E−08 | 41.60297619 | Hyper-methylated | PCa\|NT DMRs |
| chr18 | 5635801 | 5761429 | 0.000987641 | −29.20925926 | Hypo-methylated | PCa\|NT DMRs |
| chr18 | 5976988 | 6113039 | 1.88E−07 | −25.18484848 | Hypo-methylated | PCa\|NT DMRs |
| chr18 | 6252140 | 6268263 | 6.42E−05 | −21.95238095 | Hypo-methylated | PCa\|NT DMRs |
| chr18 | 6413960 | 6414460 | 1.54E−11 | 27.59716312 | Hyper-methylated | PCa\|NT DMRs |
| chr18 | 8479988 | 8480174 | 0.001969641 | −41.60714286 | Hypo-methylated | PCa\|NT DMRs |
| chr18 | 8706693 | 8707546 | 6.35E−16 | 21.01549509 | Hyper-methylated | PCa\|NT DMRs |
| chr18 | 8990408 | 8990902 | 1.97E−05 | −40.68315018 | Hypo-methylated | PCa\|NT DMRs |
| chr18 | 9404630 | 9404870 | 1.97E−05 | 57.23809524 | Hyper-methylated | PCa\|NT DMRs |
| chr18 | 9434059 | 9434604 | 0.000252289 | 33.14557823 | Hyper-methylated | PCa\|NT DMRs |
| chr18 | 9735310 | 9735576 | 0.000205174 | 62.1452381 | Hyper-methylated | PCa\|NT DMRs |
| chr18 | 9994457 | 9994840 | 1.11E−05 | −25.95238095 | Hypo-methylated | PCa\|NT DMRs |
| chr18 | 10031031 | 10031433 | 1.91E−05 | −29.28823529 | Hypo-methylated | PCa\|NT DMRs |
| chr18 | 10044815 | 10045873 | 5.94E−06 | −28.79220779 | Hypo-methylated | PCa\|NT DMRs |
| chr18 | 10154168 | 10165815 | 4.11E−07 | −25.73082707 | Hypo-methylated | PCa\|NT DMRs |
| chr18 | 10205334 | 10205458 | 0.001969641 | −40.38095238 | Hypo-methylated | PCa\|NT DMRs |
| chr18 | 11912911 | 11913228 | 0.000205174 | −44.7202381 | Hypo-methylated | PCa\|NT DMRs |
| chr18 | 11947339 | 11947990 | 6.98E−09 | 36.44109347 | Hyper-methylated | PCa\|NT DMRs |
| chr18 | 11972539 | 11973184 | 4.45E−08 | 47.08984127 | Hyper-methylated | PCa\|NT DMRs |
| chr18 | 12030311 | 12038759 | 1.23E−07 | 30.60260771 | Hyper-methylated | PCa\|NT DMRs |
| chr18 | 12254366 | 12258991 | 0.001789867 | 23.57812098 | Hyper-methylated | PCa\|NT DMRs |
| chr18 | 12304718 | 12305349 | 1.97E−05 | −50.91857143 | Hypo-methylated | PCa\|NT DMRs |
| chr18 | 13125696 | 13127628 | 0.000133627 | −43.75272109 | Hypo-methylated | PCa\|NT DMRs |
| chr18 | 13609838 | 13612631 | 5.61E−06 | 29.99293924 | Hyper-methylated | PCa\|NT DMRs |
| chr18 | 14089936 | 14090087 | 0.003622801 | −45.35714286 | Hypo-methylated | PCa\|NT DMRs |
| chr18 | 18820939 | 18821478 | 0.000551093 | 27.49166667 | Hyper-methylated | PCa\|NT DMRs |
| chr18 | 19928268 | 19928436 | 0.00064571 | 25.63265306 | Hyper-methylated | PCa\|NT DMRs |
| chr18 | 20139815 | 20140198 | 3.43E−06 | 20.00744048 | Hyper-methylated | PCa\|NT DMRs |
| chr18 | 20910586 | 20945671 | 0.00046742 | −46.10978836 | Hypo-methylated | PCa\|NT DMRs |
| chr18 | 21538184 | 21538279 | 0.005968871 | −56.72380952 | Hypo-methylated | PCa\|NT DMRs |
| chr18 | 24276356 | 24276514 | 0.000205174 | −55.82738095 | Hypo-methylated | PCa\|NT DMRs |
| chr18 | 25228330 | 25253283 | 8.96E−05 | −29.74484127 | Hypo-methylated | PCa\|NT DMRs |
| chr18 | 27932428 | 28466836 | 1.45E−22 | −34.74115646 | Hypo-methylated | PCa\|NT DMRs |
| chr18 | 28620847 | 28622543 | 3.99E−07 | 30.00595238 | Hyper-methylated | PCa\|NT DMRs |
| chr18 | 29077638 | 29077795 | 9.90E−08 | 34.74489796 | Hyper-methylated | PCa\|NT DMRs |
| chr18 | 30352209 | 30352557 | 1.17E−05 | 26.52089947 | Hyper-methylated | PCa\|NT DMRs |
| chr18 | 30442296 | 30509521 | 2.29E−06 | −22.46580087 | Hypo-methylated | PCa\|NT DMRs |
| chr18 | 31323339 | 31390167 | 0.003448944 | −32.72176871 | Hypo-methylated | PCa\|NT DMRs |
| chr18 | 31390368 | 31400593 | 8.49E−05 | 20.85460317 | Hyper-methylated | PCa\|NT DMRs |
| chr18 | 32792152 | 32813276 | 0.001012507 | −23.67518797 | Hypo-methylated | PCa\|NT DMRs |
| chr18 | 32847409 | 32851161 | 0.0005791 | 28.64173669 | Hyper-methylated | PCa\|NT DMRs |
| chr18 | 34020215 | 34020487 | 0.002585277 | 29.01488095 | Hyper-methylated | PCa\|NT DMRs |
| chr18 | 34252375 | 34274307 | 0.007428376 | −31.61794872 | Hypo-methylated | PCa\|NT DMRs |
| chr18 | 34940110 | 34942444 | 0.001969641 | 27.85142857 | Hyper-methylated | PCa\|NT DMRs |
| chr18 | 35394696 | 35608880 | 4.84E−06 | −29.72312925 | Hypo-methylated | PCa\|NT DMRs |
| chr18 | 38126083 | 38360861 | 4.90E−08 | −28.0805383 | Hypo-methylated | PCa\|NT DMRs |
| chr18 | 38443201 | 38446621 | 0.001969641 | −27.76587302 | Hypo-methylated | PCa\|NT DMRs |
| chr18 | 42205310 | 42205381 | 0.005968871 | 24.84285714 | Hyper-methylated | PCa\|NT DMRs |
| chr18 | 42260979 | 42261339 | 5.84E−05 | 36.83690476 | Hyper-methylated | PCa\|NT DMRs |
| chr18 | 43729751 | 43729860 | 0.005968871 | 39.48952381 | Hyper-methylated | PCa\|NT DMRs |
| chr18 | 44000356 | 44007468 | 0.001969641 | −20.61768707 | Hypo-methylated | PCa\|NT DMRs |
| chr18 | 44083744 | 44119233 | 9.63E−06 | −23.72040816 | Hypo-methylated | PCa\|NT DMRs |
| chr18 | 45052748 | 45088031 | 1.12E−05 | 27.18872692 | Hyper-methylated | PCa\|NT DMRs |
| chr18 | 45663439 | 45664546 | 2.25E−06 | 22.96245421 | Hyper-methylated | PCa\|NT DMRs |
| chr18 | 45885472 | 45902852 | 0.007679542 | 23.14285714 | Hyper-methylated | PCa\|NT DMRs |
| chr18 | 45967896 | 45968273 | 6.92E−09 | 35.71652661 | Hyper-methylated | PCa\|NT DMRs |
| chr18 | 46140263 | 46142694 | 0.000279943 | −40.35714286 | Hypo-methylated | PCa\|NT DMRs |
| chr18 | 46269517 | 46277092 | 0.005513661 | 26.04081633 | Hyper-methylated | PCa\|NT DMRs |
| chr18 | 46298127 | 46298252 | 2.86E−05 | 55.07875458 | Hyper-methylated | PCa\|NT DMRs |
| chr18 | 46307558 | 46307907 | 8.29E−07 | 39.23809524 | Hyper-methylated | PCa\|NT DMRs |
| chr18 | 46378023 | 46378134 | 6.42E−05 | 44.83280423 | Hyper-methylated | PCa\|NT DMRs |
| chr18 | 46423165 | 46428480 | 0.002346655 | 33.04952381 | Hyper-methylated | PCa\|NT DMRs |
| chr18 | 46449021 | 46453935 | 0.000900958 | 25.91011905 | Hyper-methylated | PCa\|NT DMRs |
| chr18 | 46469134 | 46474220 | 0.00248539 | −27.05952381 | Hypo-methylated | PCa\|NT DMRs |
| chr18 | 46989638 | 46989709 | 0.003622801 | −37.3015873 | Hypo-methylated | PCa\|NT DMRs |
| chr18 | 47655124 | 47655428 | 0.002585277 | −22.03869048 | Hypo-methylated | PCa\|NT DMRs |
| chr18 | 47808950 | 47809166 | 0.000205174 | −56.75 | Hypo-methylated | PCa\|NT DMRs |
| chr18 | 47825115 | 47825150 | 3.32E−06 | 30.00992063 | Hyper-methylated | PCa\|NT DMRs |
| chr18 | 47926679 | 47927797 | 0.000516919 | 21.6280112 | Hyper-methylated | PCa\|NT DMRs |
| chr18 | 55020895 | 55020977 | 6.29E−05 | 21.37678571 | Hyper-methylated | PCa\|NT DMRs |
| chr18 | 55103505 | 55104185 | 6.07E−15 | 28.77847619 | Hyper-methylated | PCa\|NT DMRs |
| chr18 | 55862725 | 55862837 | 0.003949155 | 25.6547619 | Hyper-methylated | PCa\|NT DMRs |
| chr18 | 55866457 | 55888853 | 8.96E−05 | −34.6 | Hypo-methylated | PCa\|NT DMRs |
| chr18 | 55914971 | 55983264 | 9.19E−17 | −33.72566586 | Hypo-methylated | PCa\|NT DMRs |
| chr18 | 56435928 | 56459491 | 0.000916248 | −30.58809524 | Hypo-methylated | PCa\|NT DMRs |
| chr18 | 56534826 | 56534925 | 0.005513661 | −21.73809524 | Hypo-methylated | PCa\|NT DMRs |
| chr18 | 59221458 | 59221582 | 4.96E−06 | 24.05970696 | Hyper-methylated | PCa\|NT DMRs |
| chr18 | 60283708 | 60284081 | 4.96E−06 | −22.99157509 | Hypo-methylated | PCa\|NT DMRs |
| chr18 | 60805457 | 60805774 | 0.001969641 | 52.49206349 | Hyper-methylated | PCa\|NT DMRs |
| chr18 | 60872466 | 60874723 | 0.000836132 | −30.16137566 | Hypo-methylated | PCa\|NT DMRs |
| chr18 | 61693760 | 62049292 | 2.63E−07 | −28.17708333 | Hypo-methylated | PCa\|NT DMRs |
| chr18 | 62341460 | 62388735 | 0.001480644 | −25.30666667 | Hypo-methylated | PCa\|NT DMRs |
| chr18 | 62799996 | 63051094 | 7.75E−09 | −32.64039409 | Hypo-methylated | PCa\|NT DMRs |
| chr18 | 63941738 | 64696309 | 7.64E−25 | −32.02869048 | Hypo-methylated | PCa\|NT DMRs |
| chr18 | 65397856 | 65409883 | 3.75E−05 | −23.05039683 | Hypo-methylated | PCa\|NT DMRs |
| chr18 | 66398373 | 67055347 | 4.72E−13 | −32.81855159 | Hypo-methylated | PCa\|NT DMRs |
| chr18 | 68235017 | 68584210 | 8.86E−12 | −35.13515406 | Hypo-methylated | PCa\|NT DMRs |
| chr18 | 68695627 | 69162967 | 1.50E−24 | −32.88127706 | Hypo-methylated | PCa\|NT DMRs |
| chr18 | 71864931 | 71865239 | 0.00023371 | −38.9 | Hypo-methylated | PCa\|NT DMRs |
| chr18 | 72191913 | 72191982 | 0.000205174 | 37.44047619 | Hyper-methylated | PCa\|NT DMRs |
| chr18 | 72843391 | 72845789 | 1.25E−05 | −39.57518797 | Hypo-methylated | PCa\|NT DMRs |
| chr18 | 73103349 | 73103490 | 0.005968871 | 32.84285714 | Hyper-methylated | PCa\|NT DMRs |
| chr18 | 73147711 | 73147792 | 0.001969641 | −22.23809524 | Hypo-methylated | PCa\|NT DMRs |
| chr18 | 73207047 | 73263168 | 1.70E−09 | −29.72380952 | Hypo-methylated | PCa\|NT DMRs |
| chr18 | 74060619 | 74063016 | 1.89E−10 | −25.09663866 | Hypo-methylated | PCa\|NT DMRs |
| chr18 | 74100758 | 74101207 | 0.00023371 | −27.97619048 | Hypo-methylated | PCa\|NT DMRs |
| chr18 | 74101933 | 74102108 | 0.001969641 | 30.83333333 | Hyper-methylated | PCa\|NT DMRs |
| chr18 | 74102609 | 74102752 | 0.000205174 | −32.39285714 | Hypo-methylated | PCa\|NT DMRs |
| chr18 | 74156220 | 74161241 | 0.007560755 | −25.3025974 | Hypo-methylated | PCa\|NT DMRs |
| chr18 | 74168557 | 74171800 | 0.000340264 | −24.03184524 | Hypo-methylated | PCa\|NT DMRs |
| chr18 | 74177635 | 74178207 | 6.42E−05 | −33.87037037 | Hypo-methylated | PCa\|NT DMRs |
| chr18 | 74237436 | 74240997 | 0.001789867 | −26.85978836 | Hypo-methylated | PCa\|NT DMRs |
| chr18 | 74260396 | 74271539 | 1.44E−08 | −30.62807018 | Hypo-methylated | PCa\|NT DMRs |
| chr18 | 74302669 | 74329804 | 6.67E−10 | −35.3997558 | Hypo-methylated | PCa\|NT DMRs |
| chr18 | 74359525 | 74420364 | 3.26E−18 | −31.78115359 | Hypo-methylated | PCa\|NT DMRs |
| chr18 | 74451757 | 74471188 | 8.31E−07 | −26.08596491 | Hypo-methylated | PCa\|NT DMRs |
| chr18 | 74485103 | 74489301 | 0.00023371 | −32.27857143 | Hypo-methylated | PCa\|NT DMRs |
| chr18 | 74501062 | 74505197 | 6.96E−08 | −26.53602484 | Hypo-methylated | PCa\|NT DMRs |
| chr18 | 74532349 | 74532765 | 5.52E−05 | −34.10544218 | Hypo-methylated | PCa\|NT DMRs |
| chr18 | 74639935 | 74668181 | 0.000187876 | −35.94571429 | Hypo-methylated | PCa\|NT DMRs |
| chr18 | 74717117 | 74718685 | 0.000970317 | 28.72005013 | Hyper-methylated | PCa\|NT DMRs |
| chr18 | 74764569 | 74770186 | 0.002802996 | 25.23558201 | Hyper-methylated | PCa\|NT DMRs |
| chr18 | 74811532 | 74831307 | 1.73E−05 | 39.09942857 | Hyper-methylated | PCa\|NT DMRs |
| chr18 | 74845689 | 74846836 | 0.000279449 | 36.77838828 | Hyper-methylated | PCa\|NT DMRs |
| chr18 | 74974925 | 75361403 | 4.84E−35 | −33.00436782 | Hypo-methylated | PCa\|NT DMRs |
| chr18 | 75988277 | 75989287 | 2.34E−06 | −25.54209524 | Hypo-methylated | PCa\|NT DMRs |
| chr18 | 76001773 | 76066502 | 4.25E−12 | −40.5877551 | Hypo-methylated | PCa\|NT DMRs |
| chr18 | 76397834 | 76398344 | 1.32E−11 | −23.85030525 | Hypo-methylated | PCa\|NT DMRs |
| chr18 | 76686037 | 76694294 | 7.83E−12 | −29.80888031 | Hypo-methylated | PCa\|NT DMRs |
| chr18 | 76696627 | 76701402 | 1.76E−06 | −35.34444444 | Hypo-methylated | PCa\|NT DMRs |
| chr18 | 76732737 | 76733245 | 0.000847246 | 21.2514881 | Hyper-methylated | PCa\|NT DMRs |
| chr18 | 76771436 | 76771765 | 0.000377317 | −33.36607143 | Hypo-methylated | PCa\|NT DMRs |
| chr18 | 76809069 | 76819347 | 0.004095673 | −23.38015873 | Hypo-methylated | PCa\|NT DMRs |
| chr18 | 77164214 | 77164402 | 0.00068166 | −33.29464286 | Hypo-methylated | PCa\|NT DMRs |
| chr18 | 77165070 | 77166405 | 0.006962527 | 24.77922078 | Hyper-methylated | PCa\|NT DMRs |
| chr18 | 77230973 | 77231391 | 2.86E−05 | −26.37728938 | Hypo-methylated | PCa\|NT DMRs |
| chr18 | 77269146 | 77271155 | 0.005250907 | 24.71428571 | Hyper-methylated | PCa\|NT DMRs |
| chr18 | 77286196 | 77286514 | 0.000138104 | −40.89010989 | Hypo-methylated | PCa\|NT DMRs |
| chr18 | 77286792 | 77286863 | 0.005968871 | 45.05714286 | Hyper-methylated | PCa\|NT DMRs |
| chr18 | 77305815 | 77306020 | 0.000111427 | 31.64285714 | Hyper-methylated | PCa\|NT DMRs |
| chr18 | 77326996 | 77327149 | 0.000840726 | 25.54497354 | Hyper-methylated | PCa\|NT DMRs |
| chr18 | 77377287 | 77378031 | 2.28E−10 | 31.24540421 | Hyper-methylated | PCa\|NT DMRs |
| chr18 | 77385419 | 77385522 | 0.000117699 | 44.78268398 | Hyper-methylated | PCa\|NT DMRs |
| chr18 | 77397396 | 77398552 | 6.37E−17 | 29.2531746 | Hyper-methylated | PCa\|NT DMRs |
| chr18 | 77401928 | 77403666 | 9.63E−06 | 24.98503401 | Hyper-methylated | PCa\|NT DMRs |
| chr18 | 77417058 | 77421389 | 9.82E−05 | −37.43785714 | Hypo-methylated | PCa\|NT DMRs |
| chr18 | 77513564 | 77513610 | 0.003622801 | 30.06349206 | Hyper-methylated | PCa\|NT DMRs |
| chr18 | 77550113 | 77552761 | 5.52E−05 | 40.15612245 | Hyper-methylated | PCa\|NT DMRs |
| chr18 | 77585927 | 77587079 | 0.001387751 | 21.38888889 | Hyper-methylated | PCa\|NT DMRs |
| chr18 | 77624182 | 77624643 | 0.001419801 | −22.89801587 | Hypo-methylated | PCa\|NT DMRs |
| chr18 | 77645189 | 77645464 | 2.86E−05 | −33.14652015 | Hypo-methylated | PCa\|NT DMRs |
| chr18 | 77701958 | 77702130 | 0.005968871 | −46.71428571 | Hypo-methylated | PCa\|NT DMRs |
| chr18 | 77749201 | 77749269 | 0.00068166 | −25.5297619 | Hypo-methylated | PCa\|NT DMRs |
| chr18 | 77917521 | 77918406 | 1.35E−10 | 22.29748918 | Hyper-methylated | PCa\|NT DMRs |
| chr19 | 371152 | 378497 | 2.00E−06 | −23.41666667 | Hypo-methylated | PCa\|NT DMRs |
| chr19 | 378512 | 384409 | 0.000836113 | 21.26478405 | Hyper-methylated | PCa\|NT DMRs |
| chr19 | 402190 | 405795 | 0.001255204 | −21.92883598 | Hypo-methylated | PCa\|NT DMRs |
| chr19 | 408140 | 408687 | 1.88E−05 | 29.99333333 | Hyper-methylated | PCa\|NT DMRs |
| chr19 | 447294 | 448407 | 7.67E−06 | 20.92712843 | Hyper-methylated | PCa\|NT DMRs |
| chr19 | 478295 | 478505 | 0.000413298 | −23.54761905 | Hypo-methylated | PCa\|NT DMRs |
| chr19 | 510096 | 512297 | 0.002614618 | 30.35238095 | Hyper-methylated | PCa\|NT DMRs |
| chr19 | 518100 | 519234 | 1.10E−15 | 24.23426704 | Hyper-methylated | PCa\|NT DMRs |
| chr19 | 530194 | 530751 | 0.002534928 | −34.85714286 | Hypo-methylated | PCa\|NT DMRs |
| chr19 | 548955 | 551325 | 0.000267776 | −22.86434379 | Hypo-methylated | PCa\|NT DMRs |
| chr19 | 560234 | 560348 | 0.000481535 | 46.91005291 | Hyper-methylated | PCa\|NT DMRs |
| chr19 | 584816 | 584981 | 1.76E−06 | −26.36309524 | Hypo-methylated | PCa\|NT DMRs |
| chr19 | 601919 | 602003 | 0.000205174 | −28.57142857 | Hypo-methylated | PCa\|NT DMRs |
| chr19 | 681323 | 681990 | 4.33E−08 | 43.19092262 | Hyper-methylated | PCa\|NT DMRs |
| chr19 | 695352 | 696175 | 1.11E−05 | 33.18673469 | Hyper-methylated | PCa\|NT DMRs |
| chr19 | 709640 | 710259 | 5.77E−08 | 22.90509031 | Hyper-methylated | PCa\|NT DMRs |
| chr19 | 718532 | 725613 | 1.48E−18 | 22.54185185 | Hyper-methylated | PCa\|NT DMRs |
| chr19 | 728020 | 728703 | 6.09E−07 | 29.52857143 | Hyper-methylated | PCa\|NT DMRs |
| chr19 | 757465 | 757591 | 0.001181579 | 28.84013605 | Hyper-methylated | PCa\|NT DMRs |
| chr19 | 782402 | 783442 | 8.88E−07 | −38.90142857 | Hypo-methylated | PCa\|NT DMRs |
| chr19 | 799341 | 803541 | 2.67E−05 | −31.15526316 | Hypo-methylated | PCa\|NT DMRs |
| chr19 | 822785 | 825238 | 1.37E−06 | 31.03061224 | Hyper-methylated | PCa\|NT DMRs |
| chr19 | 827950 | 831864 | 2.05E−11 | 27.48715986 | Hyper-methylated | PCa\|NT DMRs |
| chr19 | 835599 | 836772 | 0.00046638 | 33.76785714 | Hyper-methylated | PCa\|NT DMRs |
| chr19 | 839383 | 841111 | 1.21E−07 | 34.06371882 | Hyper-methylated | PCa\|NT DMRs |
| chr19 | 846101 | 846650 | 0.008581782 | 20.56626984 | Hyper-methylated | PCa\|NT DMRs |
| chr19 | 854165 | 855286 | 0.00641359 | 26.8531746 | Hyper-methylated | PCa\|NT DMRs |
| chr19 | 859395 | 859606 | 0.0019106 | 29.60912698 | Hyper-methylated | PCa\|NT DMRs |
| chr19 | 932153 | 933525 | 3.00E−10 | 38.27777778 | Hyper-methylated | PCa\|NT DMRs |
| chr19 | 947398 | 949464 | 4.49E−10 | 32.19115646 | Hyper-methylated | PCa\|NT DMRs |
| chr19 | 1007773 | 1007858 | 0.00023371 | −25.44155844 | Hypo-methylated | PCa\|NT DMRs |
| chr19 | 1018844 | 1019992 | 0.000358233 | −25.04195011 | Hypo-methylated | PCa\|NT DMRs |
| chr19 | 1034196 | 1039775 | 6.42E−05 | 25.3923436 | Hyper-methylated | PCa\|NT DMRs |
| chr19 | 1067618 | 1069625 | 2.77E−06 | 35.63080357 | Hyper-methylated | PCa\|NT DMRs |
| chr19 | 1070598 | 1071921 | 2.08E−23 | −47.11936937 | Hypo-methylated | PCa\|NT DMRs |
| chr19 | 1075175 | 1075599 | 0.003622801 | −29.02380952 | Hypo-methylated | PCa\|NT DMRs |
| chr19 | 1086852 | 1088697 | 0.001021878 | 30.52063492 | Hyper-methylated | PCa\|NT DMRs |
| chr19 | 1101810 | 1102102 | 0.000480383 | 48.91798942 | Hyper-methylated | PCa\|NT DMRs |
| chr19 | 1136308 | 1136738 | 2.06E−05 | 29.54428571 | Hyper-methylated | PCa\|NT DMRs |
| chr19 | 1159985 | 1160266 | 0.000239173 | 23.00250627 | Hyper-methylated | PCa\|NT DMRs |
| chr19 | 1161048 | 1162181 | 0.006589535 | −31.86190476 | Hypo-methylated | PCa\|NT DMRs |
| chr19 | 1176597 | 1177816 | 7.21E−08 | 28.76904762 | Hyper-methylated | PCa\|NT DMRs |
| chr19 | 1242192 | 1243429 | 4.02E−06 | −29.53586626 | Hypo-methylated | PCa\|NT DMRs |
| chr19 | 1262222 | 1262382 | 0.007959104 | −23.22108844 | Hypo-methylated | PCa\|NT DMRs |
| chr19 | 1265942 | 1266420 | 1.09E−07 | 23.61591479 | Hyper-methylated | PCa\|NT DMRs |
| chr19 | 1271418 | 1273576 | 9.86E−11 | −40.40735931 | Hypo-methylated | PCa\|NT DMRs |
| chr19 | 1274244 | 1275277 | 4.01E−05 | −32.19595238 | Hypo-methylated | PCa\|NT DMRs |
| chr19 | 1285212 | 1285734 | 8.99E−07 | 22.62380952 | Hyper-methylated | PCa\|NT DMRs |
| chr19 | 1287464 | 1288163 | 9.85E−10 | 31.49880952 | Hyper-methylated | PCa\|NT DMRs |
| chr19 | 1288639 | 1289667 | 1.10E−06 | −38.79276896 | Hypo-methylated | PCa\|NT DMRs |
| chr19 | 1313033 | 1313733 | 3.52E−06 | −38.08110119 | Hypo-methylated | PCa\|NT DMRs |
| chr19 | 1361935 | 1365290 | 1.62E−05 | 43.31690476 | Hyper-methylated | PCa\|NT DMRs |
| chr19 | 1402314 | 1403326 | 2.02E−05 | 33.21329365 | Hyper-methylated | PCa\|NT DMRs |
| chr19 | 1406473 | 1406675 | 0.003034084 | −28.2829932 | Hypo-methylated | PCa\|NT DMRs |
| chr19 | 1409232 | 1413512 | 2.03E−06 | −25.5335277 | Hypo-methylated | PCa\|NT DMRs |
| chr19 | 1423630 | 1423980 | 6.15E−05 | 32.34693878 | Hyper-methylated | PCa\|NT DMRs |
| chr19 | 1428919 | 1430074 | 3.09E−13 | −24.95111759 | Hypo-methylated | PCa\|NT DMRs |
| chr19 | 1439387 | 1442204 | 3.11E−06 | −36.11198157 | Hypo-methylated | PCa\|NT DMRs |
| chr19 | 1444782 | 1445172 | 0.000832876 | 21.55734127 | Hyper-methylated | PCa\|NT DMRs |
| chr19 | 1465543 | 1468222 | 8.35E−36 | 23.71452888 | Hyper-methylated | PCa\|NT DMRs |
| chr19 | 1488871 | 1489961 | 0.001917734 | 25.87900433 | Hyper-methylated | PCa\|NT DMRs |
| chr19 | 1560401 | 1560483 | 0.005513661 | 31.65178571 | Hyper-methylated | PCa\|NT DMRs |
| chr19 | 1566459 | 1567001 | 6.62E−07 | −22.68571429 | Hypo-methylated | PCa\|NT DMRs |
| chr19 | 1608801 | 1609277 | 1.97E−05 | 46.47714286 | Hyper-methylated | PCa\|NT DMRs |
| chr19 | 1650686 | 1651372 | 1.18E−05 | 26.80333333 | Hyper-methylated | PCa\|NT DMRs |
| chr19 | 1807647 | 1807781 | 0.001969641 | −47.3015873 | Hypo-methylated | PCa\|NT DMRs |
| chr19 | 1851350 | 1852902 | 5.20E−05 | 26.40445469 | Hyper-methylated | PCa\|NT DMRs |
| chr19 | 1871300 | 1871440 | 0.004095673 | −55.8531746 | Hypo-methylated | PCa\|NT DMRs |
| chr19 | 1873159 | 1873842 | 2.86E−06 | −42.75062657 | Hypo-methylated | PCa\|NT DMRs |
| chr19 | 1895160 | 1896611 | 0.002572399 | 42.63003663 | Hyper-methylated | PCa\|NT DMRs |
| chr19 | 1902083 | 1902433 | 0.001969641 | 33.24285714 | Hyper-methylated | PCa\|NT DMRs |
| chr19 | 1909735 | 1910292 | 0.001036892 | 23.48049887 | Hyper-methylated | PCa\|NT DMRs |
| chr19 | 1942876 | 1943965 | 0.004722489 | 20.05161905 | Hyper-methylated | PCa\|NT DMRs |
| chr19 | 1966065 | 1967497 | 2.11E−05 | 20.50414079 | Hyper-methylated | PCa\|NT DMRs |
| chr19 | 2039373 | 2040317 | 1.44E−05 | −20.46571429 | Hypo-methylated | PCa\|NT DMRs |
| chr19 | 2042730 | 2048391 | 6.14E−06 | −22.88179272 | Hypo-methylated | PCa\|NT DMRs |
| chr19 | 2187846 | 2188096 | 0.000481535 | 47.99470899 | Hyper-methylated | PCa\|NT DMRs |
| chr19 | 2282230 | 2282727 | 2.65E−07 | 37.35862069 | Hyper-methylated | PCa\|NT DMRs |
| chr19 | 2287824 | 2289683 | 1.32E−06 | 30.80083333 | Hyper-methylated | PCa\|NT DMRs |
| chr19 | 2291020 | 2293774 | 1.66E−23 | −29.37114846 | Hypo-methylated | PCa\|NT DMRs |
| chr19 | 2302307 | 2303568 | 4.49E−13 | 36.91047619 | Hyper-methylated | PCa\|NT DMRs |
| chr19 | 2345491 | 2345665 | 0.00641359 | 28.88095238 | Hyper-methylated | PCa\|NT DMRs |
| chr19 | 2354910 | 2359677 | 0.002529619 | −22.81648352 | Hypo-methylated | PCa\|NT DMRs |
| chr19 | 2385087 | 2390403 | 0.004164881 | −23.08902692 | Hypo-methylated | PCa\|NT DMRs |
| chr19 | 2424058 | 2424686 | 7.04E−12 | 28.32987013 | Hyper-methylated | PCa\|NT DMRs |
| chr19 | 2459860 | 2460148 | 0.004175963 | 37.89380952 | Hyper-methylated | PCa\|NT DMRs |
| chr19 | 2485702 | 2489206 | 1.35E−07 | 28.4218845 | Hyper-methylated | PCa\|NT DMRs |
| chr19 | 2556146 | 2556599 | 0.001907648 | 24.51190476 | Hyper-methylated | PCa\|NT DMRs |
| chr19 | 2560196 | 2567879 | 9.55E−05 | 34.53601954 | Hyper-methylated | PCa\|NT DMRs |
| chr19 | 2588128 | 2588480 | 0.00064571 | −59.65510204 | Hypo-methylated | PCa\|NT DMRs |
| chr19 | 2602607 | 2603432 | 0.001651948 | −44.16428571 | Hypo-methylated | PCa\|NT DMRs |
| chr19 | 2616695 | 2622934 | 8.64E−11 | 29.11208113 | Hyper-methylated | PCa\|NT DMRs |
| chr19 | 2774775 | 2777106 | 0.003325063 | 22.76482335 | Hyper-methylated | PCa\|NT DMRs |
| chr19 | 2951723 | 2953384 | 0.009050681 | −36.64761905 | Hypo-methylated | PCa\|NT DMRs |
| chr19 | 2987049 | 2987498 | 0.000320955 | 23.11904762 | Hyper-methylated | PCa\|NT DMRs |
| chr19 | 3012871 | 3013791 | 5.77E−05 | −29.91792717 | Hypo-methylated | PCa\|NT DMRs |
| chr19 | 3138355 | 3146717 | 2.37E−05 | 30.29365079 | Hyper-methylated | PCa\|NT DMRs |
| chr19 | 3178378 | 3180413 | 7.37E−21 | 53.68178382 | Hyper-methylated | PCa\|NT DMRs |
| chr19 | 3187917 | 3190958 | 1.22E−07 | 23.74834055 | Hyper-methylated | PCa\|NT DMRs |
| chr19 | 3238074 | 3252530 | 1.09E−12 | −26.23852814 | Hypo-methylated | PCa\|NT DMRs |
| chr19 | 3264014 | 3269456 | 3.67E−06 | −30.29619048 | Hypo-methylated | PCa\|NT DMRs |
| chr19 | 3313259 | 3314118 | 2.09E−05 | −27.2032381 | Hypo-methylated | PCa\|NT DMRs |
| chr19 | 3317793 | 3327553 | 0.000348785 | −21.34313725 | Hypo-methylated | PCa\|NT DMRs |
| chr19 | 3358286 | 3358744 | 1.75E−11 | 29.22453102 | Hyper-methylated | PCa\|NT DMRs |
| chr19 | 3374732 | 3376007 | 9.81E−08 | −37.57744361 | Hypo-methylated | PCa\|NT DMRs |
| chr19 | 3381483 | 3383444 | 2.64E−14 | −42.01149068 | Hypo-methylated | PCa\|NT DMRs |
| chr19 | 3428520 | 3428833 | 3.00E−05 | 28.23469388 | Hyper-methylated | PCa\|NT DMRs |
| chr19 | 3478582 | 3480252 | 0.005438982 | 29.71428571 | Hyper-methylated | PCa\|NT DMRs |
| chr19 | 3482628 | 3483658 | 3.33E−16 | −49.66025267 | Hypo-methylated | PCa\|NT DMRs |
| chr19 | 3511710 | 3512025 | 0.000632596 | −32.56926407 | Hypo-methylated | PCa\|NT DMRs |
| chr19 | 3516500 | 3516933 | 0.008649834 | −20.1625 | Hypo-methylated | PCa\|NT DMRs |
| chr19 | 3574382 | 3574512 | 2.81E−09 | −41.76648352 | Hypo-methylated | PCa\|NT DMRs |
| chr19 | 3604804 | 3605248 | 4.45E−08 | 54.52857143 | Hyper-methylated | PCa\|NT DMRs |
| chr19 | 3607029 | 3608789 | 3.27E−05 | 20.22236025 | Hyper-methylated | PCa\|NT DMRs |
| chr19 | 3617535 | 3618176 | 6.63E−09 | 45.16007326 | Hyper-methylated | PCa\|NT DMRs |
| chr19 | 3670411 | 3673683 | 2.66E−06 | 26.97888889 | Hyper-methylated | PCa\|NT DMRs |
| chr19 | 3683811 | 3684036 | 0.000652115 | 32.53516484 | Hyper-methylated | PCa\|NT DMRs |
| chr19 | 3710212 | 3710355 | 0.001583613 | −40.88961039 | Hypo-methylated | PCa\|NT DMRs |
| chr19 | 3718829 | 3718907 | 0.000215808 | −31.73015873 | Hypo-methylated | PCa\|NT DMRs |
| chr19 | 3807390 | 3809019 | 8.19E−05 | −30.504329 | Hypo-methylated | PCa\|NT DMRs |
| chr19 | 3819013 | 3819252 | 1.88E−05 | −21.65449735 | Hypo-methylated | PCa\|NT DMRs |
| chr19 | 3907690 | 3908173 | 6.90E−07 | −22.52321429 | Hypo-methylated | PCa\|NT DMRs |
| chr19 | 3948043 | 3951920 | 9.11E−06 | 35.28941799 | Hyper-methylated | PCa\|NT DMRs |
| chr19 | 3966265 | 3967186 | 1.12E−07 | −29.73219048 | Hypo-methylated | PCa\|NT DMRs |
| chr19 | 3977640 | 3978160 | 3.33E−11 | −31.31529582 | Hypo-methylated | PCa\|NT DMRs |
| chr19 | 3979239 | 3979393 | 0.008482405 | −21.01809524 | Hypo-methylated | PCa\|NT DMRs |
| chr19 | 3981115 | 3983190 | 6.42E−05 | −35.31534392 | Hypo-methylated | PCa\|NT DMRs |
| chr19 | 4015736 | 4015812 | 0.003622801 | 22.84920635 | Hyper-methylated | PCa\|NT DMRs |
| chr19 | 4038770 | 4045696 | 2.08E−10 | −32.49554044 | Hypo-methylated | PCa\|NT DMRs |
| chr19 | 4046926 | 4046984 | 0.001181579 | −24.55102041 | Hypo-methylated | PCa\|NT DMRs |
| chr19 | 4058067 | 4058586 | 0.002651286 | 21.59693878 | Hyper-methylated | PCa\|NT DMRs |
| chr19 | 4068070 | 4068751 | 0.000679335 | 22.80304762 | Hyper-methylated | PCa\|NT DMRs |
| chr19 | 4086455 | 4086794 | 0.00010418 | −34.80986395 | Hypo-methylated | PCa\|NT DMRs |
| chr19 | 4103193 | 4103246 | 0.00064571 | 30.16462585 | Hyper-methylated | PCa\|NT DMRs |
| chr19 | 4108812 | 4109207 | 6.94E−06 | 59.03555556 | Hyper-methylated | PCa\|NT DMRs |
| chr19 | 4163482 | 4168379 | 0.001394173 | −28.98231293 | Hypo-methylated | PCa\|NT DMRs |
| chr19 | 4198205 | 4198602 | 7.28E−11 | 21.66383929 | Hyper-methylated | PCa\|NT DMRs |
| chr19 | 4301832 | 4302494 | 0.00064571 | 55.40816327 | Hyper-methylated | PCa\|NT DMRs |
| chr19 | 4344571 | 4344619 | 0.008482405 | 52.83809524 | Hyper-methylated | PCa\|NT DMRs |
| chr19 | 4369323 | 4370878 | 4.58E−07 | 23.83088023 | Hyper-methylated | PCa\|NT DMRs |
| chr19 | 4373772 | 4374700 | 7.28E−07 | 35.05639098 | Hyper-methylated | PCa\|NT DMRs |
| chr19 | 4518533 | 4518598 | 0.000305995 | −26.86190476 | Hypo-methylated | PCa\|NT DMRs |
| chr19 | 4536655 | 4538264 | 0.000524456 | −28.80995671 | Hypo-methylated | PCa\|NT DMRs |
| chr19 | 4542602 | 4544477 | 5.88E−25 | 38.73634322 | Hyper-methylated | PCa\|NT DMRs |
| chr19 | 4556161 | 4558124 | 0.000348677 | −24.39103641 | Hypo-methylated | PCa\|NT DMRs |
| chr19 | 4559566 | 4559839 | 0.004095673 | 50.1 | Hyper-methylated | PCa\|NT DMRs |
| chr19 | 4567138 | 4567482 | 3.00E−05 | −46.75102041 | Hypo-methylated | PCa\|NT DMRs |
| chr19 | 4620229 | 4624238 | 5.52E−05 | −38.80519481 | Hypo-methylated | PCa\|NT DMRs |
| chr19 | 4713714 | 4717697 | 2.28E−08 | −45.64262485 | Hypo-methylated | PCa\|NT DMRs |
| chr19 | 4772470 | 4787253 | 0.001969641 | −25.25833333 | Hypo-methylated | PCa\|NT DMRs |
| chr19 | 4800720 | 4804755 | 0.000957967 | −31.95362319 | Hypo-methylated | PCa\|NT DMRs |
| chr19 | 4807778 | 4808386 | 0.001344447 | 43.01785714 | Hyper-methylated | PCa\|NT DMRs |
| chr19 | 4877969 | 4878424 | 9.61E−05 | −39.47767857 | Hypo-methylated | PCa\|NT DMRs |
| chr19 | 4878461 | 4878713 | 0.000377317 | 36.97916667 | Hyper-methylated | PCa\|NT DMRs |
| chr19 | 4950551 | 4950963 | 0.000112123 | 42.05357143 | Hyper-methylated | PCa\|NT DMRs |
| chr19 | 5046482 | 5048428 | 3.62E−05 | −24.28611111 | Hypo-methylated | PCa\|NT DMRs |
| chr19 | 5074460 | 5075377 | 0.003762238 | 21.56666667 | Hyper-methylated | PCa\|NT DMRs |
| chr19 | 5130467 | 5131295 | 8.94E−07 | 34.0377551 | Hyper-methylated | PCa\|NT DMRs |
| chr19 | 5139347 | 5139556 | 0.000480383 | 42.31534392 | Hyper-methylated | PCa\|NT DMRs |
| chr19 | 5205514 | 5205583 | 0.00064571 | 26.36734694 | Hyper-methylated | PCa\|NT DMRs |
| chr19 | 5215656 | 5218706 | 0.003919956 | 20.71596639 | Hyper-methylated | PCa\|NT DMRs |
| chr19 | 5250114 | 5251558 | 3.44E−06 | −38.2452381 | Hypo-methylated | PCa\|NT DMRs |
| chr19 | 5269515 | 5269626 | 0.001969641 | −52.9047619 | Hypo-methylated | PCa\|NT DMRs |
| chr19 | 5294036 | 5294417 | 2.40E−05 | 23.62698413 | Hyper-methylated | PCa\|NT DMRs |
| chr19 | 5322403 | 5322661 | 0.000145171 | 32.5085034 | Hyper-methylated | PCa\|NT DMRs |
| chr19 | 5335747 | 5339130 | 1.03E−06 | 37.4046332 | Hyper-methylated | PCa\|NT DMRs |
| chr19 | 5418110 | 5426591 | 0.002185514 | −25.8218254 | Hypo-methylated | PCa\|NT DMRs |
| chr19 | 5448575 | 5450624 | 0.00064571 | −37.08163265 | Hypo-methylated | PCa\|NT DMRs |
| chr19 | 5499096 | 5499475 | 0.001969641 | 36.84126984 | Hyper-methylated | PCa\|NT DMRs |
| chr19 | 5621525 | 5621773 | 0.000974795 | −35.85714286 | Hypo-methylated | PCa\|NT DMRs |
| chr19 | 5686442 | 5686569 | 0.007959104 | 34.59251701 | Hyper-methylated | PCa\|NT DMRs |
| chr19 | 5838153 | 5839226 | 0.008506146 | 28.1025641 | Hyper-methylated | PCa\|NT DMRs |
| chr19 | 5894674 | 5894896 | 1.34E−05 | 25.06870748 | Hyper-methylated | PCa\|NT DMRs |
| chr19 | 5905591 | 5905798 | 0.005186056 | −33.45714286 | Hypo-methylated | PCa\|NT DMRs |
| chr19 | 5914451 | 5916316 | 6.65E−06 | −29.42031746 | Hypo-methylated | PCa\|NT DMRs |
| chr19 | 5945815 | 5953965 | 0.004095673 | 35.83630952 | Hyper-methylated | PCa\|NT DMRs |
| chr19 | 6114636 | 6114690 | 0.005968871 | −25.49809524 | Hypo-methylated | PCa\|NT DMRs |
| chr19 | 6129055 | 6137081 | 0.00051204 | −34.82857143 | Hypo-methylated | PCa\|NT DMRs |
| chr19 | 6144622 | 6154136 | 2.86E−05 | −21.93003663 | Hypo-methylated | PCa\|NT DMRs |
| chr19 | 6270202 | 6273136 | 1.92E−05 | −21.07518797 | Hypo-methylated | PCa\|NT DMRs |
| chr19 | 6333548 | 6333658 | 0.00068166 | −46.35714286 | Hypo-methylated | PCa\|NT DMRs |
| chr19 | 6376545 | 6378720 | 5.52E−05 | −20.75324675 | Hypo-methylated | PCa\|NT DMRs |
| chr19 | 6417812 | 6418059 | 0.000205174 | −33.16845238 | Hypo-methylated | PCa\|NT DMRs |
| chr19 | 6481647 | 6481833 | 0.000205174 | 27.04940476 | Hyper-methylated | PCa\|NT DMRs |
| chr19 | 6697435 | 6697472 | 0.005968871 | 50.57142857 | Hyper-methylated | PCa\|NT DMRs |
| chr19 | 6710805 | 6711076 | 0.000908257 | 21.97985348 | Hyper-methylated | PCa\|NT DMRs |
| chr19 | 6724750 | 6730808 | 9.82E−07 | −26.81987578 | Hypo-methylated | PCa\|NT DMRs |
| chr19 | 6744609 | 6750343 | 5.76E−05 | −35.18492063 | Hypo-methylated | PCa\|NT DMRs |
| chr19 | 7101062 | 7101628 | 0.001115302 | −42.13392857 | Hypo-methylated | PCa\|NT DMRs |
| chr19 | 7254767 | 7266253 | 0.001969641 | −37.21825397 | Hypo-methylated | PCa\|NT DMRs |
| chr19 | 7267671 | 7267734 | 6.42E−05 | −32.65343915 | Hypo-methylated | PCa\|NT DMRs |
| chr19 | 7543280 | 7543862 | 4.37E−05 | −37.94957983 | Hypo-methylated | PCa\|NT DMRs |
| chr19 | 7547753 | 7549196 | 0.008482405 | 24.77777778 | Hyper-methylated | PCa\|NT DMRs |
| chr19 | 7592700 | 7592858 | 0.001969641 | 43.54421769 | Hyper-methylated | PCa\|NT DMRs |
| chr19 | 7615579 | 7616039 | 0.000226704 | −22.12698413 | Hypo-methylated | PCa\|NT DMRs |
| chr19 | 7709745 | 7711614 | 1.57E−06 | 24.61904762 | Hyper-methylated | PCa\|NT DMRs |
| chr19 | 7727512 | 7729091 | 0.004649531 | −30.53673469 | Hypo-methylated | PCa\|NT DMRs |
| chr19 | 7733401 | 7734144 | 0.000909347 | 32.19047619 | Hyper-methylated | PCa\|NT DMRs |
| chr19 | 7796262 | 7810767 | 0.000145907 | −23.63214286 | Hypo-methylated | PCa\|NT DMRs |
| chr19 | 7870592 | 7870720 | 0.000205174 | −30.4827381 | Hypo-methylated | PCa\|NT DMRs |
| chr19 | 7918539 | 7918932 | 0.000191452 | −34.01904762 | Hypo-methylated | PCa\|NT DMRs |
| chr19 | 7959672 | 7960059 | 5.87E−05 | −30.88541667 | Hypo-methylated | PCa\|NT DMRs |
| chr19 | 7983777 | 7984641 | 0.0002059 | 22.02724868 | Hyper-methylated | PCa\|NT DMRs |
| chr19 | 8048603 | 8052184 | 0.00641359 | −24.1631746 | Hypo-methylated | PCa\|NT DMRs |
| chr19 | 8212110 | 8212815 | 0.007959104 | −26.75238095 | Hypo-methylated | PCa\|NT DMRs |
| chr19 | 8285132 | 8290802 | 0.001969641 | −34.3047619 | Hypo-methylated | PCa\|NT DMRs |
| chr19 | 8347921 | 8362790 | 0.001969641 | 49.77777778 | Hyper-methylated | PCa\|NT DMRs |
| chr19 | 8405741 | 8405903 | 0.001969641 | 34.5952381 | Hyper-methylated | PCa\|NT DMRs |
| chr19 | 8407336 | 8408048 | 4.06E−11 | 23.83996265 | Hyper-methylated | PCa\|NT DMRs |
| chr19 | 8563629 | 8568649 | 5.08E−08 | 28.72363946 | Hyper-methylated | PCa\|NT DMRs |
| chr19 | 8641619 | 8641790 | 0.001969641 | 48.38095238 | Hyper-methylated | PCa\|NT DMRs |
| chr19 | 8648734 | 8649840 | 0.000205174 | −29.39583333 | Hypo-methylated | PCa\|NT DMRs |
| chr19 | 8808017 | 8808271 | 5.70E−06 | −28.68779762 | Hypo-methylated | PCa\|NT DMRs |
| chr19 | 8850037 | 8900701 | 6.09E−06 | −33.87468672 | Hypo-methylated | PCa\|NT DMRs |
| chr19 | 8959595 | 9094957 | 2.89E−10 | −26.82419825 | Hypo-methylated | PCa\|NT DMRs |
| chr19 | 9121025 | 9129101 | 0.000186125 | −24.69863946 | Hypo-methylated | PCa\|NT DMRs |
| chr19 | 9193322 | 9245738 | 2.11E−06 | −27.74807256 | Hypo-methylated | PCa\|NT DMRs |
| chr19 | 9324971 | 9341980 | 0.000836132 | −29.67089947 | Hypo-methylated | PCa\|NT DMRs |
| chr19 | 9594179 | 9600248 | 0.000570576 | −25.46296296 | Hypo-methylated | PCa\|NT DMRs |
| chr19 | 9811212 | 9811410 | 0.001969641 | −41.33333333 | Hypo-methylated | PCa\|NT DMRs |
| chr19 | 10023944 | 10024122 | 6.16E−06 | 27.3141321 | Hyper-methylated | PCa\|NT DMRs |
| chr19 | 10197524 | 10197962 | 3.91E−08 | 33.24055829 | Hyper-methylated | PCa\|NT DMRs |
| chr19 | 10212648 | 10216685 | 2.12E−05 | 32.39880952 | Hyper-methylated | PCa\|NT DMRs |
| chr19 | 10348462 | 10350007 | 0.000205174 | −20.82738095 | Hypo-methylated | PCa\|NT DMRs |
| chr19 | 10415932 | 10416154 | 0.000205174 | 38.83333333 | Hyper-methylated | PCa\|NT DMRs |
| chr19 | 10427154 | 10427523 | 0.000680799 | −23.62619048 | Hypo-methylated | PCa\|NT DMRs |
| chr19 | 10444751 | 10446562 | 3.30E−15 | −40.16089873 | Hypo-methylated | PCa\|NT DMRs |
| chr19 | 10621079 | 10627982 | 2.56E−13 | 34.48672032 | Hyper-methylated | PCa\|NT DMRs |
| chr19 | 10655170 | 10655622 | 2.87E−07 | −36.90625 | Hypo-methylated | PCa\|NT DMRs |
| chr19 | 10728282 | 10728944 | 0.00064571 | −41.55646259 | Hypo-methylated | PCa\|NT DMRs |
| chr19 | 10729827 | 10736595 | 4.86E−05 | −45.60612245 | Hypo-methylated | PCa\|NT DMRs |
| chr19 | 10913426 | 10916550 | 0.001775064 | 33.55357143 | Hyper-methylated | PCa\|NT DMRs |
| chr19 | 11006529 | 11008429 | 0.008576577 | −22.41333333 | Hypo-methylated | PCa\|NT DMRs |
| chr19 | 11009808 | 11010367 | 0.000205174 | −52.17857143 | Hypo-methylated | PCa\|NT DMRs |
| chr19 | 11046776 | 11049788 | 0.007959104 | −21.00340136 | Hypo-methylated | PCa\|NT DMRs |
| chr19 | 11129600 | 11129858 | 0.001969641 | −43.06666667 | Hypo-methylated | PCa\|NT DMRs |
| chr19 | 11222535 | 11225019 | 0.001115302 | −25.075 | Hypo-methylated | PCa\|NT DMRs |
| chr19 | 11253445 | 11256750 | 0.000747867 | 25.72732919 | Hyper-methylated | PCa\|NT DMRs |
| chr19 | 11275865 | 11276631 | 0.001083007 | −42.96 | Hypo-methylated | PCa\|NT DMRs |
| chr19 | 11290781 | 11290880 | 0.00064571 | 41.2244898 | Hyper-methylated | PCa\|NT DMRs |
| chr19 | 11346278 | 11346423 | 0.001969641 | −26.88095238 | Hypo-methylated | PCa\|NT DMRs |
| chr19 | 11510773 | 11517387 | 1.37E−06 | 30.8956044 | Hyper-methylated | PCa\|NT DMRs |
| chr19 | 11533973 | 11534787 | 0.002178049 | 26.70238095 | Hyper-methylated | PCa\|NT DMRs |
| chr19 | 11561958 | 11563491 | 0.003764982 | −20.23214286 | Hypo-methylated | PCa\|NT DMRs |
| chr19 | 11689380 | 11695537 | 7.09E−06 | 23.63492063 | Hyper-methylated | PCa\|NT DMRs |
| chr19 | 12799867 | 12801000 | 1.15E−10 | 39.12310924 | Hyper-methylated | PCa\|NT DMRs |
| chr19 | 12809953 | 12810119 | 0.005968871 | 44.51428571 | Hyper-methylated | PCa\|NT DMRs |
| chr19 | 12861100 | 12863961 | 7.57E−06 | −30.94334975 | Hypo-methylated | PCa\|NT DMRs |
| chr19 | 12991878 | 12992096 | 4.45E−06 | 37.02255639 | Hyper-methylated | PCa\|NT DMRs |
| chr19 | 13024891 | 13025225 | 0.001969641 | 31.39285714 | Hyper-methylated | PCa\|NT DMRs |
| chr19 | 13135415 | 13170789 | 9.28E−07 | −22.23027569 | Hypo-methylated | PCa\|NT DMRs |
| chr19 | 13187907 | 13188162 | 0.009807302 | 29.96666667 | Hyper-methylated | PCa\|NT DMRs |
| chr19 | 13190529 | 13192536 | 0.007959104 | 21.35034014 | Hyper-methylated | PCa\|NT DMRs |
| chr19 | 13278440 | 13283579 | 6.50E−05 | 37.99338624 | Hyper-methylated | PCa\|NT DMRs |
| chr19 | 13318581 | 13319534 | 4.92E−08 | 21.4155102 | Hyper-methylated | PCa\|NT DMRs |
| chr19 | 13334496 | 13338154 | 3.10E−06 | −26.37338936 | Hypo-methylated | PCa\|NT DMRs |
| chr19 | 13354790 | 13356130 | 0.00068166 | −22.59047619 | Hypo-methylated | PCa\|NT DMRs |
| chr19 | 13378118 | 13392280 | 0.000138013 | −21.50019841 | Hypo-methylated | PCa\|NT DMRs |
| chr19 | 13737577 | 13805027 | 9.70E−13 | −28.52210012 | Hypo-methylated | PCa\|NT DMRs |
| chr19 | 13892166 | 13895619 | 0.007628253 | −32.63265306 | Hypo-methylated | PCa\|NT DMRs |
| chr19 | 13957399 | 13957836 | 2.50E−05 | 30.10204082 | Hyper-methylated | PCa\|NT DMRs |
| chr19 | 13985009 | 13988520 | 1.18E−07 | −40.88280423 | Hypo-methylated | PCa\|NT DMRs |
| chr19 | 14048637 | 14049519 | 1.25E−05 | 22.07238095 | Hyper-methylated | PCa\|NT DMRs |
| chr19 | 14172637 | 14173098 | 5.49E−06 | −34.84117647 | Hypo-methylated | PCa\|NT DMRs |
| chr19 | 14275032 | 14275847 | 2.40E−06 | −25.12296919 | Hypo-methylated | PCa\|NT DMRs |
| chr19 | 14278533 | 14278695 | 0.005968871 | 36.34285714 | Hyper-methylated | PCa\|NT DMRs |
| chr19 | 14549802 | 14551213 | 0.000215808 | 49.0984127 | Hyper-methylated | PCa\|NT DMRs |
| chr19 | 14590249 | 14595415 | 0.000878681 | 22.97910906 | Hyper-methylated | PCa\|NT DMRs |
| chr19 | 14664087 | 14664476 | 0.001060348 | −33.16796537 | Hypo-methylated | PCa\|NT DMRs |
| chr19 | 14672903 | 14673401 | 1.05E−06 | 25.17232143 | Hyper-methylated | PCa\|NT DMRs |
| chr19 | 14682129 | 14682334 | 0.009407512 | −25.07936508 | Hypo-methylated | PCa\|NT DMRs |
| chr19 | 15093063 | 15103275 | 0.001969641 | −37.76428571 | Hypo-methylated | PCa\|NT DMRs |
| chr19 | 15362390 | 15375514 | 0.001083007 | −23.97333333 | Hypo-methylated | PCa\|NT DMRs |
| chr19 | 15485652 | 15488971 | 0.000221601 | −35.68 | Hypo-methylated | PCa\|NT DMRs |
| chr19 | 15574654 | 15575148 | 0.001291665 | 32.36060606 | Hyper-methylated | PCa\|NT DMRs |
| chr19 | 15596467 | 15605638 | 0.005968871 | −38.57142857 | Hypo-methylated | PCa\|NT DMRs |
| chr19 | 16013127 | 16013383 | 0.000205174 | −55.61130952 | Hypo-methylated | PCa\|NT DMRs |
| chr19 | 16021999 | 16022767 | 3.20E−05 | 25.27857143 | Hyper-methylated | PCa\|NT DMRs |
| chr19 | 16178195 | 16179234 | 2.67E−06 | −40.45238095 | Hypo-methylated | PCa\|NT DMRs |
| chr19 | 16240467 | 16260740 | 0.00717477 | 23.69547619 | Hyper-methylated | PCa\|NT DMRs |
| chr19 | 16349971 | 16350040 | 0.005968871 | 42.88571429 | Hyper-methylated | PCa\|NT DMRs |
| chr19 | 16369475 | 16370071 | 2.87E−07 | 42.40892857 | Hyper-methylated | PCa\|NT DMRs |
| chr19 | 16370711 | 16374643 | 3.80E−05 | 29.90895238 | Hyper-methylated | PCa\|NT DMRs |
| chr19 | 16436257 | 16436488 | 8.46E−09 | 30.67454844 | Hyper-methylated | PCa\|NT DMRs |
| chr19 | 16436517 | 16438118 | 1.31E−14 | 25.92214286 | Hyper-methylated | PCa\|NT DMRs |
| chr19 | 16469794 | 16474415 | 1.87E−07 | −35.62775041 | Hypo-methylated | PCa\|NT DMRs |
| chr19 | 16553536 | 16572854 | 0.003410216 | 24.22118227 | Hyper-methylated | PCa\|NT DMRs |
| chr19 | 16679409 | 16681355 | 6.46E−05 | −42.67927171 | Hypo-methylated | PCa\|NT DMRs |
| chr19 | 16740662 | 16741101 | 0.002437742 | −25.48677249 | Hypo-methylated | PCa\|NT DMRs |
| chr19 | 16997017 | 16997386 | 6.42E−05 | −52.04444444 | Hypo-methylated | PCa\|NT DMRs |
| chr19 | 16998131 | 17000172 | 2.05E−08 | 32.50805861 | Hyper-methylated | PCa\|NT DMRs |
| chr19 | 17000416 | 17000553 | 0.00641359 | −35.16666667 | Hypo-methylated | PCa\|NT DMRs |
| chr19 | 17220811 | 17223138 | 0.000238933 | 40.76134454 | Hyper-methylated | PCa\|NT DMRs |
| chr19 | 17295465 | 17295652 | 5.84E−05 | 31.91269841 | Hyper-methylated | PCa\|NT DMRs |
| chr19 | 17432633 | 17434853 | 7.63E−06 | 27.89943978 | Hyper-methylated | PCa\|NT DMRs |
| chr19 | 17435675 | 17435887 | 2.50E−05 | 24.71428571 | Hyper-methylated | PCa\|NT DMRs |
| chr19 | 17436824 | 17436925 | 0.008482405 | −56.4 | Hypo-methylated | PCa\|NT DMRs |
| chr19 | 17439195 | 17444730 | 1.38E−10 | 28.28034188 | Hyper-methylated | PCa\|NT DMRs |
| chr19 | 17525215 | 17526515 | 0.000354065 | −24.05291005 | Hypo-methylated | PCa\|NT DMRs |
| chr19 | 17577605 | 17579893 | 0.000532147 | −29.36160714 | Hypo-methylated | PCa\|NT DMRs |
| chr19 | 17611054 | 17611276 | 4.43E−05 | 35.53846154 | Hyper-methylated | PCa\|NT DMRs |
| chr19 | 17750311 | 17754804 | 0.001241245 | −24.24603175 | Hypo-methylated | PCa\|NT DMRs |
| chr19 | 17798795 | 17799015 | 0.000260936 | 20.32857143 | Hyper-methylated | PCa\|NT DMRs |
| chr19 | 17801261 | 17811490 | 0.000264944 | −25.95914787 | Hypo-methylated | PCa\|NT DMRs |
| chr19 | 17822460 | 17826085 | 0.003314163 | −20.30095238 | Hypo-methylated | PCa\|NT DMRs |
| chr19 | 17862385 | 17862689 | 0.001969641 | 50.93650794 | Hyper-methylated | PCa\|NT DMRs |
| chr19 | 17865032 | 17867890 | 0.002437742 | −43.73174603 | Hypo-methylated | PCa\|NT DMRs |
| chr19 | 17889612 | 17889687 | 0.000117699 | 20.58961039 | Hyper-methylated | PCa\|NT DMRs |
| chr19 | 17895037 | 17899476 | 3.50E−07 | −35.64130952 | Hypo-methylated | PCa\|NT DMRs |
| chr19 | 17918738 | 17919098 | 1.69E−06 | 40.25378151 | Hyper-methylated | PCa\|NT DMRs |
| chr19 | 17952174 | 17953860 | 7.57E−14 | −44.66057767 | Hypo-methylated | PCa\|NT DMRs |
| chr19 | 17956861 | 17961721 | 3.70E−07 | 36.47037037 | Hyper-methylated | PCa\|NT DMRs |
| chr19 | 17972294 | 17972423 | 0.00064571 | −36.07823129 | Hypo-methylated | PCa\|NT DMRs |
| chr19 | 17979885 | 17981185 | 0.001836557 | −37.6014652 | Hypo-methylated | PCa\|NT DMRs |
| chr19 | 18000442 | 18016276 | 0.000458282 | −26.8 | Hypo-methylated | PCa\|NT DMRs |
| chr19 | 18103747 | 18105514 | 6.67E−08 | −37.8452381 | Hypo-methylated | PCa\|NT DMRs |
| chr19 | 18112564 | 18113834 | 0.000483473 | 42.45388471 | Hyper-methylated | PCa\|NT DMRs |
| chr19 | 18160302 | 18164219 | 1.28E−11 | −32.52571429 | Hypo-methylated | PCa\|NT DMRs |
| chr19 | 18241272 | 18241477 | 0.005968871 | −39.31428571 | Hypo-methylated | PCa\|NT DMRs |
| chr19 | 18280215 | 18280875 | 3.45E−06 | 35.44047619 | Hyper-methylated | PCa\|NT DMRs |
| chr19 | 18337226 | 18337259 | 0.001969641 | 25.24489796 | Hyper-methylated | PCa\|NT DMRs |
| chr19 | 18385724 | 18389226 | 0.001076343 | −38.34415584 | Hypo-methylated | PCa\|NT DMRs |
| chr19 | 18402128 | 18402488 | 0.004668266 | 36.45670996 | Hyper-methylated | PCa\|NT DMRs |
| chr19 | 18413801 | 18418259 | 0.004147932 | 22.3572536 | Hyper-methylated | PCa\|NT DMRs |
| chr19 | 18487353 | 18487402 | 0.001969641 | −37.1 | Hypo-methylated | PCa\|NT DMRs |
| chr19 | 18496953 | 18497245 | 0.003314163 | −24.89134199 | Hypo-methylated | PCa\|NT DMRs |
| chr19 | 18543841 | 18546127 | 4.57E−15 | 24.48280952 | Hyper-methylated | PCa\|NT DMRs |
| chr19 | 18547391 | 18548286 | 7.46E−07 | 27.42433862 | Hyper-methylated | PCa\|NT DMRs |
| chr19 | 18575494 | 18575762 | 0.004095673 | −27.37777778 | Hypo-methylated | PCa\|NT DMRs |
| chr19 | 18681567 | 18681778 | 0.004095673 | −37.64285714 | Hypo-methylated | PCa\|NT DMRs |
| chr19 | 18714318 | 18716768 | 8.85E−08 | 24.22438095 | Hyper-methylated | PCa\|NT DMRs |
| chr19 | 18760599 | 18761133 | 3.64E−09 | 34.03669468 | Hyper-methylated | PCa\|NT DMRs |
| chr19 | 18811957 | 18812199 | 0.000149006 | 23.7 | Hyper-methylated | PCa\|NT DMRs |
| chr19 | 18901520 | 18902284 | 4.03E−18 | 27.05335628 | Hyper-methylated | PCa\|NT DMRs |
| chr19 | 18905090 | 18906588 | 0.000374142 | 38.72380952 | Hyper-methylated | PCa\|NT DMRs |
| chr19 | 18951016 | 18960412 | 0.001352148 | −25.53112245 | Hypo-methylated | PCa\|NT DMRs |
| chr19 | 18979694 | 18981748 | 2.18E−39 | 39.42739927 | Hyper-methylated | PCa\|NT DMRs |
| chr19 | 19063548 | 19063652 | 0.00064571 | 21.24489796 | Hyper-methylated | PCa\|NT DMRs |
| chr19 | 19147116 | 19157167 | 0.000104747 | −40.22321429 | Hypo-methylated | PCa\|NT DMRs |
| chr19 | 19271495 | 19271710 | 0.003290817 | 38.84464286 | Hyper-methylated | PCa\|NT DMRs |
| chr19 | 19283026 | 19283081 | 0.003622801 | 41.43730159 | Hyper-methylated | PCa\|NT DMRs |
| chr19 | 19491645 | 19492619 | 0.000118733 | −40.93650794 | Hypo-methylated | PCa\|NT DMRs |
| chr19 | 19571532 | 19572810 | 0.007904371 | 26.40505952 | Hyper-methylated | PCa\|NT DMRs |
| chr19 | 19651498 | 19651882 | 1.66E−05 | 21.09943978 | Hyper-methylated | PCa\|NT DMRs |
| chr19 | 19653211 | 19654259 | 0.000214897 | 37.62783883 | Hyper-methylated | PCa\|NT DMRs |
| chr19 | 19778818 | 19778994 | 0.000205174 | 54.33928571 | Hyper-methylated | PCa\|NT DMRs |
| chr19 | 20278004 | 20278616 | 1.18E−08 | 30.81845238 | Hyper-methylated | PCa\|NT DMRs |
| chr19 | 20348686 | 20349228 | 6.93E−06 | 45.61111111 | Hyper-methylated | PCa\|NT DMRs |
| chr19 | 20606250 | 20607581 | 2.15E−07 | 31.74557823 | Hyper-methylated | PCa\|NT DMRs |
| chr19 | 20843255 | 20843844 | 0.001992179 | −44.47142857 | Hypo-methylated | PCa\|NT DMRs |
| chr19 | 20847353 | 20847508 | 0.005968871 | 56.88571429 | Hyper-methylated | PCa\|NT DMRs |
| chr19 | 21623174 | 21623405 | 0.000233379 | −34.67142857 | Hypo-methylated | PCa\|NT DMRs |
| chr19 | 22123798 | 22181253 | 0.000377317 | −34.11904762 | Hypo-methylated | PCa\|NT DMRs |
| chr19 | 22490402 | 22550924 | 5.84E−05 | −31.26984127 | Hypo-methylated | PCa\|NT DMRs |
| chr19 | 22802992 | 22803360 | 0.005968871 | −40.42857143 | Hypo-methylated | PCa\|NT DMRs |
| chr19 | 22886078 | 22886440 | 4.11E−07 | −29.15162907 | Hypo-methylated | PCa\|NT DMRs |
| chr19 | 23120722 | 23159340 | 0.00023371 | −39.27714286 | Hypo-methylated | PCa\|NT DMRs |
| chr19 | 23227552 | 23245488 | 0.005968871 | −26.84761905 | Hypo-methylated | PCa\|NT DMRs |
| chr19 | 23432262 | 23432362 | 0.001241245 | 23.78042328 | Hyper-methylated | PCa\|NT DMRs |
| chr19 | 28127248 | 28148388 | 0.003464153 | −27.0995671 | Hypo-methylated | PCa\|NT DMRs |
| chr19 | 30086741 | 30089433 | 0.005513661 | −34.20535714 | Hypo-methylated | PCa\|NT DMRs |
| chr19 | 30166557 | 30169447 | 0.000176046 | −41.88961039 | Hypo-methylated | PCa\|NT DMRs |
| chr19 | 30941973 | 30942241 | 1.41E−06 | −31.22987013 | Hypo-methylated | PCa\|NT DMRs |
| chr19 | 30986767 | 30987216 | 0.00064571 | −29.3414966 | Hypo-methylated | PCa\|NT DMRs |
| chr19 | 31321711 | 31321998 | 0.001969641 | −45.18253968 | Hypo-methylated | PCa\|NT DMRs |
| chr19 | 31474520 | 31555683 | 0.001969641 | −23.74285714 | Hypo-methylated | PCa\|NT DMRs |
| chr19 | 31826312 | 31839968 | 1.18E−09 | 23.75385488 | Hyper-methylated | PCa\|NT DMRs |
| chr19 | 32714895 | 32715500 | 1.88E−09 | 34.75977444 | Hyper-methylated | PCa\|NT DMRs |
| chr19 | 32801697 | 32820005 | 0.005968871 | −20.4 | Hypo-methylated | PCa\|NT DMRs |
| chr19 | 32898351 | 32898516 | 0.005968871 | −50.53714286 | Hypo-methylated | PCa\|NT DMRs |
| chr19 | 33014138 | 33028623 | 0.00641359 | −25.08174603 | Hypo-methylated | PCa\|NT DMRs |
| chr19 | 33167677 | 33168890 | 0.001836557 | 31 | Hyper-methylated | PCa\|NT DMRs |
| chr19 | 33462065 | 33462322 | 0.000145094 | −26.91984127 | Hypo-methylated | PCa\|NT DMRs |
| chr19 | 33493262 | 33494136 | 0.007560755 | −28.73214286 | Hypo-methylated | PCa\|NT DMRs |
| chr19 | 33528506 | 33528757 | 0.00064571 | 45.38095238 | Hyper-methylated | PCa\|NT DMRs |
| chr19 | 33703571 | 33705583 | 6.64E−06 | −22.44658385 | Hypo-methylated | PCa\|NT DMRs |
| chr19 | 33738253 | 33740439 | 1.41E−06 | 47.6890873 | Hyper-methylated | PCa\|NT DMRs |
| chr19 | 33863333 | 33863367 | 0.001969641 | 52.16269841 | Hyper-methylated | PCa\|NT DMRs |
| chr19 | 34175937 | 34176092 | 0.001969641 | −30.52380952 | Hypo-methylated | PCa\|NT DMRs |
| chr19 | 34190969 | 34191046 | 0.005968871 | −24.62857143 | Hypo-methylated | PCa\|NT DMRs |
| chr19 | 34269364 | 34270600 | 0.000159126 | −32.30392157 | Hypo-methylated | PCa\|NT DMRs |
| chr19 | 34288917 | 34289092 | 0.000397381 | 28.86813187 | Hyper-methylated | PCa\|NT DMRs |
| chr19 | 34397576 | 34398063 | 2.01E−07 | 39.61547619 | Hyper-methylated | PCa\|NT DMRs |
| chr19 | 34990698 | 34992342 | 5.22E−06 | 27.00620491 | Hyper-methylated | PCa\|NT DMRs |
| chr19 | 35329641 | 35330050 | 2.37E−05 | 30.0015873 | Hyper-methylated | PCa\|NT DMRs |
| chr19 | 35513606 | 35514698 | 0.004187875 | 22.17904762 | Hyper-methylated | PCa\|NT DMRs |
| chr19 | 35529289 | 35530483 | 6.42E−05 | 51.38624339 | Hyper-methylated | PCa\|NT DMRs |
| chr19 | 35571361 | 35571936 | 0.004505524 | −22.72619048 | Hypo-methylated | PCa\|NT DMRs |
| chr19 | 35589653 | 35589780 | 0.001969641 | −46.29444444 | Hypo-methylated | PCa\|NT DMRs |
| chr19 | 35617536 | 35622220 | 4.92E−05 | −32.45445135 | Hypo-methylated | PCa\|NT DMRs |
| chr19 | 35630057 | 35630425 | 0.001969641 | 37.53412698 | Hyper-methylated | PCa\|NT DMRs |
| chr19 | 35646137 | 35646705 | 3.45E−06 | 27.78548753 | Hyper-methylated | PCa\|NT DMRs |
| chr19 | 35776898 | 35786457 | 1.86E−05 | −30.05357143 | Hypo-methylated | PCa\|NT DMRs |
| chr19 | 35786470 | 35786913 | 4.08E−07 | 43.64172336 | Hyper-methylated | PCa\|NT DMRs |
| chr19 | 35867066 | 35889791 | 0.000918789 | −34.93373016 | Hypo-methylated | PCa\|NT DMRs |
| chr19 | 36034454 | 36036245 | 1.05E−06 | 20.21031746 | Hyper-methylated | PCa\|NT DMRs |
| chr19 | 36076491 | 36095407 | 0.00179283 | −27.83333333 | Hypo-methylated | PCa\|NT DMRs |
| chr19 | 36156676 | 36162056 | 0.000106046 | −25.38388278 | Hypo-methylated | PCa\|NT DMRs |
| chr19 | 36164216 | 36164312 | 0.001607793 | 20.10170068 | Hyper-methylated | PCa\|NT DMRs |
| chr19 | 36247064 | 36247623 | 4.08E−05 | 22.3527619 | Hyper-methylated | PCa\|NT DMRs |
| chr19 | 36288748 | 36288946 | 0.000481535 | 25.96825397 | Hyper-methylated | PCa\|NT DMRs |
| chr19 | 36389354 | 36389595 | 0.00046742 | 25.23246753 | Hyper-methylated | PCa\|NT DMRs |
| chr19 | 36425236 | 36426349 | 0.001969641 | −31.25 | Hypo-methylated | PCa\|NT DMRs |
| chr19 | 36456308 | 36473550 | 7.18E−06 | −27.83460317 | Hypo-methylated | PCa\|NT DMRs |
| chr19 | 36497964 | 36499748 | 2.28E−05 | −36.21794872 | Hypo-methylated | PCa\|NT DMRs |
| chr19 | 36523147 | 36526376 | 8.87E−08 | 27.75608466 | Hyper-methylated | PCa\|NT DMRs |
| chr19 | 36623220 | 36630256 | 0.002082381 | −25.39635854 | Hypo-methylated | PCa\|NT DMRs |
| chr19 | 36643236 | 36645673 | 2.09E−05 | 30.6727619 | Hyper-methylated | PCa\|NT DMRs |
| chr19 | 36736022 | 36736839 | 7.02E−16 | 34.61350826 | Hyper-methylated | PCa\|NT DMRs |
| chr19 | 37287620 | 37287787 | 0.008511532 | 22.78717949 | Hyper-methylated | PCa\|NT DMRs |
| chr19 | 37719008 | 37719105 | 0.00064571 | −40.95918367 | Hypo-methylated | PCa\|NT DMRs |
| chr19 | 38321899 | 38344642 | 4.57E−08 | −36.26488095 | Hypo-methylated | PCa\|NT DMRs |
| chr19 | 38700251 | 38705221 | 1.82E−10 | 28.42932331 | Hyper-methylated | PCa\|NT DMRs |
| chr19 | 38712862 | 38713495 | 5.06E−13 | 39.16243386 | Hyper-methylated | PCa\|NT DMRs |
| chr19 | 38734003 | 38736437 | 0.00603903 | −29.94021164 | Hypo-methylated | PCa\|NT DMRs |
| chr19 | 38741729 | 38742036 | 0.001451132 | −30.25164835 | Hypo-methylated | PCa\|NT DMRs |
| chr19 | 38757037 | 38768268 | 0.001115302 | −38.47321429 | Hypo-methylated | PCa\|NT DMRs |
| chr19 | 38876420 | 38878008 | 9.21E−18 | 32.2982962 | Hyper-methylated | PCa\|NT DMRs |
| chr19 | 39018216 | 39018437 | 2.04E−05 | 22.04047619 | Hyper-methylated | PCa\|NT DMRs |
| chr19 | 39083616 | 39105102 | 4.63E−07 | −38.84093407 | Hypo-methylated | PCa\|NT DMRs |
| chr19 | 39227169 | 39228308 | 2.23E−08 | 42.62759104 | Hyper-methylated | PCa\|NT DMRs |
| chr19 | 39260650 | 39261013 | 1.18E−08 | 24.11309524 | Hyper-methylated | PCa\|NT DMRs |
| chr19 | 39466078 | 39467011 | 4.62E−14 | 23.51975684 | Hyper-methylated | PCa\|NT DMRs |
| chr19 | 39523419 | 39524207 | 0.000260936 | −36.13809524 | Hypo-methylated | PCa\|NT DMRs |
| chr19 | 39694478 | 39694549 | 0.000122432 | 20.65800866 | Hyper-methylated | PCa\|NT DMRs |
| chr19 | 39908574 | 39908686 | 4.32E−05 | 27.52014652 | Hyper-methylated | PCa\|NT DMRs |
| chr19 | 39953789 | 39954920 | 0.001181579 | −30.06802721 | Hypo-methylated | PCa\|NT DMRs |
| chr19 | 40009637 | 40009906 | 5.84E−05 | −26.58333333 | Hypo-methylated | PCa\|NT DMRs |
| chr19 | 40061407 | 40082871 | 3.39E−05 | −27.90995671 | Hypo-methylated | PCa\|NT DMRs |
| chr19 | 40264258 | 40282009 | 0.000554289 | −24.57857143 | Hypo-methylated | PCa\|NT DMRs |
| chr19 | 40329675 | 40329748 | 6.42E−05 | −32.34920635 | Hypo-methylated | PCa\|NT DMRs |
| chr19 | 40366038 | 40366240 | 6.22E−05 | −30.11394558 | Hypo-methylated | PCa\|NT DMRs |
| chr19 | 40732294 | 40732804 | 3.39E−13 | 21.29153439 | Hyper-methylated | PCa\|NT DMRs |
| chr19 | 40871618 | 40871679 | 2.83E−05 | 43.18681319 | Hyper-methylated | PCa\|NT DMRs |
| chr19 | 40904509 | 40904765 | 1.21E−07 | 33.95739348 | Hyper-methylated | PCa\|NT DMRs |
| chr19 | 40923191 | 40923426 | 0.005968871 | 48.57619048 | Hyper-methylated | PCa\|NT DMRs |
| chr19 | 40938664 | 40939515 | 0.000474458 | −36.24835165 | Hypo-methylated | PCa\|NT DMRs |
| chr19 | 41018897 | 41018956 | 4.11E−07 | 21.87218045 | Hyper-methylated | PCa\|NT DMRs |
| chr19 | 41086118 | 41086527 | 0.007560755 | 35.04112554 | Hyper-methylated | PCa\|NT DMRs |
| chr19 | 41119033 | 41121031 | 5.50E−12 | 40.30868839 | Hyper-methylated | PCa\|NT DMRs |
| chr19 | 41223405 | 41225006 | 9.80E−05 | −27.86984127 | Hypo-methylated | PCa\|NT DMRs |
| chr19 | 41312763 | 41313052 | 0.000149331 | 37.35324675 | Hyper-methylated | PCa\|NT DMRs |
| chr19 | 41836698 | 41837087 | 4.97E−06 | −28.87507003 | Hypo-methylated | PCa\|NT DMRs |
| chr19 | 41843818 | 41847924 | 3.67E−06 | −22.92404762 | Hypo-methylated | PCa\|NT DMRs |
| chr19 | 41897188 | 41899754 | 0.009106413 | 23.71428571 | Hyper-methylated | PCa\|NT DMRs |
| chr19 | 41921972 | 41927168 | 0.000331541 | 36.97402597 | Hyper-methylated | PCa\|NT DMRs |
| chr19 | 41992299 | 42011361 | 7.39E−05 | −21.33100775 | Hypo-methylated | PCa\|NT DMRs |
| chr19 | 42076877 | 42093112 | 0.003314163 | −34.68095238 | Hypo-methylated | PCa\|NT DMRs |
| chr19 | 42419879 | 42420037 | 0.005034713 | 28.85714286 | Hyper-methylated | PCa\|NT DMRs |
| chr19 | 42656558 | 42667828 | 0.000339757 | −22.83541667 | Hypo-methylated | PCa\|NT DMRs |
| chr19 | 42703350 | 42708858 | 2.21E−10 | 58.5042471 | Hyper-methylated | PCa\|NT DMRs |
| chr19 | 42712919 | 42713576 | 0.001969641 | −48.25396825 | Hypo-methylated | PCa\|NT DMRs |
| chr19 | 42785248 | 42786153 | 1.42E−05 | 29.16770186 | Hyper-methylated | PCa\|NT DMRs |
| chr19 | 42927001 | 42929067 | 1.85E−12 | 20.34285714 | Hyper-methylated | PCa\|NT DMRs |
| chr19 | 43430497 | 43519871 | 7.21E−08 | −32.48571429 | Hypo-methylated | PCa\|NT DMRs |
| chr19 | 43601062 | 43690222 | 1.06E−06 | −25.22582973 | Hypo-methylated | PCa\|NT DMRs |
| chr19 | 43715046 | 43832523 | 0.00028643 | −20.89845938 | Hypo-methylated | PCa\|NT DMRs |
| chr19 | 44111754 | 44112217 | 0.000572385 | 36.7297619 | Hyper-methylated | PCa\|NT DMRs |
| chr19 | 44313171 | 44318001 | 6.98E−05 | −28.71938776 | Hypo-methylated | PCa\|NT DMRs |
| chr19 | 45033786 | 45034000 | 0.000205174 | −47.1672619 | Hypo-methylated | PCa\|NT DMRs |
| chr19 | 45260361 | 45262537 | 2.78E−12 | −20.01241956 | Hypo-methylated | PCa\|NT DMRs |
| chr19 | 45381937 | 45382113 | 0.00064571 | −47.97959184 | Hypo-methylated | PCa\|NT DMRs |
| chr19 | 45447099 | 45447241 | 0.00023371 | 47.58809524 | Hyper-methylated | PCa\|NT DMRs |
| chr19 | 45664240 | 45666026 | 7.81E−10 | −38.21020408 | Hypo-methylated | PCa\|NT DMRs |
| chr19 | 45975735 | 45976180 | 0.000914361 | 23.05102041 | Hyper-methylated | PCa\|NT DMRs |
| chr19 | 46102734 | 46102800 | 0.007628253 | 28.12244898 | Hyper-methylated | PCa\|NT DMRs |
| chr19 | 46386083 | 46388503 | 1.35E−08 | −35.62518979 | Hypo-methylated | PCa\|NT DMRs |
| chr19 | 46503762 | 46504395 | 1.52E−07 | 42.25340136 | Hyper-methylated | PCa\|NT DMRs |
| chr19 | 46714634 | 46730048 | 0.000556997 | −23.10098522 | Hypo-methylated | PCa\|NT DMRs |
| chr19 | 46773855 | 46774547 | 0.001241245 | 32.07936508 | Hyper-methylated | PCa\|NT DMRs |
| chr19 | 46787062 | 46801217 | 0.000140031 | 33.01120448 | Hyper-methylated | PCa\|NT DMRs |
| chr19 | 46877002 | 46878175 | 0.009924186 | −28.48571429 | Hypo-methylated | PCa\|NT DMRs |
| chr19 | 46907158 | 46908576 | 7.08E−14 | 26.33525836 | Hyper-methylated | PCa\|NT DMRs |
| chr19 | 47125242 | 47126400 | 0.000564835 | −26.34238095 | Hypo-methylated | PCa\|NT DMRs |
| chr19 | 47128687 | 47134999 | 0.002437742 | 29.9031746 | Hyper-methylated | PCa\|NT DMRs |
| chr19 | 47225733 | 47226402 | 0.003464153 | 31.2021645 | Hyper-methylated | PCa\|NT DMRs |
| chr19 | 47259975 | 47260133 | 9.92E−08 | 43.12018141 | Hyper-methylated | PCa\|NT DMRs |
| chr19 | 47479848 | 47481177 | 0.001969641 | −50.03401361 | Hypo-methylated | PCa\|NT DMRs |
| chr19 | 47506363 | 47508649 | 4.65E−07 | 31.4552964 | Hyper-methylated | PCa\|NT DMRs |
| chr19 | 47598391 | 47598720 | 0.000118733 | 36.75396825 | Hyper-methylated | PCa\|NT DMRs |
| chr19 | 47617702 | 47617860 | 1.97E−05 | −40.87714286 | Hypo-methylated | PCa\|NT DMRs |
| chr19 | 47635083 | 47635313 | 0.004677148 | −22.4025974 | Hypo-methylated | PCa\|NT DMRs |
| chr19 | 47730276 | 47731854 | 8.96E−18 | 21.13639456 | Hyper-methylated | PCa\|NT DMRs |
| chr19 | 47765721 | 47765770 | 0.005968871 | 40.66666667 | Hyper-methylated | PCa\|NT DMRs |
| chr19 | 47838885 | 47840511 | 0.001969641 | 34.65714286 | Hyper-methylated | PCa\|NT DMRs |
| chr19 | 47879851 | 47880071 | 0.000377317 | 20.78571429 | Hyper-methylated | PCa\|NT DMRs |
| chr19 | 47919980 | 47920505 | 0.000574164 | 29.75064935 | Hyper-methylated | PCa\|NT DMRs |
| chr19 | 47923347 | 47924413 | 0.00641359 | 33.57301587 | Hyper-methylated | PCa\|NT DMRs |
| chr19 | 48000299 | 48003854 | 1.45E−08 | −30.01479592 | Hypo-methylated | PCa\|NT DMRs |
| chr19 | 48025138 | 48025353 | 0.005968871 | 38.4 | Hyper-methylated | PCa\|NT DMRs |
| chr19 | 48122127 | 48122640 | 0.001850915 | −46.85119048 | Hypo-methylated | PCa\|NT DMRs |
| chr19 | 48267524 | 48267826 | 0.000974795 | −45.16071429 | Hypo-methylated | PCa\|NT DMRs |
| chr19 | 48565036 | 48565312 | 0.000375274 | −25.3452381 | Hypo-methylated | PCa\|NT DMRs |
| chr19 | 48588535 | 48593927 | 0.005968871 | −52.17142857 | Hypo-methylated | PCa\|NT DMRs |
| chr19 | 48828351 | 48834221 | 0.000232923 | 29.88809524 | Hyper-methylated | PCa\|NT DMRs |
| chr19 | 48993963 | 48995094 | 0.000113183 | 22.20793651 | Hyper-methylated | PCa\|NT DMRs |
| chr19 | 49046770 | 49047001 | 0.000481535 | −39.65079365 | Hypo-methylated | PCa\|NT DMRs |
| chr19 | 49055198 | 49055683 | 8.35E−08 | −42 | Hypo-methylated | PCa\|NT DMRs |
| chr19 | 49116250 | 49119583 | 6.45E−16 | −39.1979243 | Hypo-methylated | PCa\|NT DMRs |
| chr19 | 49133883 | 49134291 | 1.71E−05 | 24.19025974 | Hyper-methylated | PCa\|NT DMRs |
| chr19 | 49216218 | 49216916 | 0.006170736 | 22.58333333 | Hyper-methylated | PCa\|NT DMRs |
| chr19 | 49232216 | 49232628 | 7.26E−10 | −27.08435374 | Hypo-methylated | PCa\|NT DMRs |
| chr19 | 49263800 | 49263986 | 0.000481535 | 47.02380952 | Hyper-methylated | PCa\|NT DMRs |
| chr19 | 49275993 | 49276180 | 0.00064571 | −57.84353741 | Hypo-methylated | PCa\|NT DMRs |
| chr19 | 49362808 | 49363161 | 0.003464153 | 21.22597403 | Hyper-methylated | PCa\|NT DMRs |
| chr19 | 49363634 | 49363921 | 7.35E−05 | −30.28126984 | Hypo-methylated | PCa\|NT DMRs |
| chr19 | 49426467 | 49426594 | 0.001969641 | −37.86904762 | Hypo-methylated | PCa\|NT DMRs |
| chr19 | 49428602 | 49428862 | 0.00641359 | −34.36904762 | Hypo-methylated | PCa\|NT DMRs |
| chr19 | 49466237 | 49468076 | 0.006207271 | 31.63412698 | Hyper-methylated | PCa\|NT DMRs |
| chr19 | 49564802 | 49565792 | 6.21E−05 | 27.87755102 | Hyper-methylated | PCa\|NT DMRs |
| chr19 | 49568737 | 49569557 | 0.004047992 | 20.30442177 | Hyper-methylated | PCa\|NT DMRs |
| chr19 | 49589976 | 49590301 | 1.76E−06 | −49.00912698 | Hypo-methylated | PCa\|NT DMRs |
| chr19 | 49719451 | 49719808 | 0.001969641 | −39.37063492 | Hypo-methylated | PCa\|NT DMRs |
| chr19 | 49841314 | 49842964 | 2.74E−10 | −32.92473118 | Hypo-methylated | PCa\|NT DMRs |
| chr19 | 49918548 | 49918601 | 0.000205174 | 45.42261905 | Hyper-methylated | PCa\|NT DMRs |
| chr19 | 49937004 | 49937224 | 0.000114755 | 30.78388278 | Hyper-methylated | PCa\|NT DMRs |
| chr19 | 49939998 | 49940044 | 0.001969641 | 23.83333333 | Hyper-methylated | PCa\|NT DMRs |
| chr19 | 50215887 | 50216762 | 0.00569382 | −20.77904762 | Hypo-methylated | PCa\|NT DMRs |
| chr19 | 50227317 | 50232237 | 0.001310211 | −24.12380952 | Hypo-methylated | PCa\|NT DMRs |
| chr19 | 50312016 | 50312821 | 8.02E−10 | 21.69146141 | Hyper-methylated | PCa\|NT DMRs |
| chr19 | 50363547 | 50364453 | 3.66E−05 | −48.4952381 | Hypo-methylated | PCa\|NT DMRs |
| chr19 | 50396731 | 50401450 | 0.00460588 | 30.10412698 | Hyper-methylated | PCa\|NT DMRs |
| chr19 | 50481363 | 50481764 | 0.005521663 | −24.41904762 | Hypo-methylated | PCa\|NT DMRs |
| chr19 | 50706335 | 50706403 | 0.004553481 | 23.05555556 | Hyper-methylated | PCa\|NT DMRs |
| chr19 | 50770354 | 50770611 | 0.00022692 | 26.98095238 | Hyper-methylated | PCa\|NT DMRs |
| chr19 | 50867971 | 50868020 | 0.008482405 | 25.05238095 | Hyper-methylated | PCa\|NT DMRs |
| chr19 | 50920149 | 50921379 | 0.004516076 | −31.59325397 | Hypo-methylated | PCa\|NT DMRs |
| chr19 | 51022642 | 51022743 | 5.82E−05 | −21.08333333 | Hypo-methylated | PCa\|NT DMRs |
| chr19 | 51340199 | 51345782 | 3.93E−05 | −34.02209524 | Hypo-methylated | PCa\|NT DMRs |
| chr19 | 51521769 | 51523206 | 4.62E−15 | 29.45134199 | Hyper-methylated | PCa\|NT DMRs |
| chr19 | 51568096 | 51568561 | 2.53E−11 | 24.20767196 | Hyper-methylated | PCa\|NT DMRs |
| chr19 | 52216773 | 52222059 | 1.23E−05 | −20.94513458 | Hypo-methylated | PCa\|NT DMRs |
| chr19 | 52469725 | 52489331 | 0.005295761 | −25.88961039 | Hypo-methylated | PCa\|NT DMRs |
| chr19 | 52995962 | 52997014 | 4.00E−08 | 27.41643991 | Hyper-methylated | PCa\|NT DMRs |
| chr19 | 53540896 | 53541139 | 1.69E−06 | 22.32492997 | Hyper-methylated | PCa\|NT DMRs |
| chr19 | 53777249 | 53798642 | 0.005968871 | −31.62698413 | Hypo-methylated | PCa\|NT DMRs |
| chr19 | 54128020 | 54132209 | 0.005513661 | 40.54464286 | Hyper-methylated | PCa\|NT DMRs |
| chr19 | 54370619 | 54370797 | 0.005633183 | 23.97959184 | Hyper-methylated | PCa\|NT DMRs |
| chr19 | 54617586 | 54617655 | 0.00064571 | −25.12244898 | Hypo-methylated | PCa\|NT DMRs |
| chr19 | 54937160 | 54943696 | 7.74E−06 | −27.82822511 | Hypo-methylated | PCa\|NT DMRs |
| chr19 | 54972053 | 54974086 | 0.002579925 | −25.38551587 | Hypo-methylated | PCa\|NT DMRs |
| chr19 | 55065801 | 55107691 | 7.37E−10 | −33.14115646 | Hypo-methylated | PCa\|NT DMRs |
| chr19 | 55596179 | 55599200 | 2.35E−22 | 34.57922999 | Hyper-methylated | PCa\|NT DMRs |
| chr19 | 55609979 | 55610202 | 0.001969641 | 37.14206349 | Hyper-methylated | PCa\|NT DMRs |
| chr19 | 55630230 | 55630699 | 1.34E−05 | 38.71428571 | Hyper-methylated | PCa\|NT DMRs |
| chr19 | 55668903 | 55669229 | 0.000441963 | −23.15037594 | Hypo-methylated | PCa\|NT DMRs |
| chr19 | 55684877 | 55684950 | 3.00E−05 | 21.59183673 | Hyper-methylated | PCa\|NT DMRs |
| chr19 | 55686316 | 55689055 | 0.006207271 | −22.7281746 | Hypo-methylated | PCa\|NT DMRs |
| chr19 | 55693469 | 55698281 | 6.90E−06 | −32.04863946 | Hypo-methylated | PCa\|NT DMRs |
| chr19 | 55708628 | 55716773 | 4.41E−05 | −21.48095238 | Hypo-methylated | PCa\|NT DMRs |
| chr19 | 55760770 | 55768901 | 3.89E−06 | 36.77258687 | Hyper-methylated | PCa\|NT DMRs |
| chr19 | 55815996 | 55816145 | 6.94E−06 | −23.15460317 | Hypo-methylated | PCa\|NT DMRs |
| chr19 | 55873668 | 55874073 | 0.001139334 | −24.46388889 | Hypo-methylated | PCa\|NT DMRs |
| chr19 | 55963619 | 55964446 | 4.51E−06 | 20.04557823 | Hyper-methylated | PCa\|NT DMRs |
| chr19 | 56002684 | 56005173 | 5.51E−05 | −20.05481728 | Hypo-methylated | PCa\|NT DMRs |
| chr19 | 56018603 | 56018762 | 0.001969641 | −32.15873016 | Hypo-methylated | PCa\|NT DMRs |
| chr19 | 56037188 | 56037287 | 0.00064571 | −30.05442177 | Hypo-methylated | PCa\|NT DMRs |
| chr19 | 56075713 | 56075917 | 1.97E−05 | −40.79428571 | Hypo-methylated | PCa\|NT DMRs |
| chr19 | 56343833 | 56347146 | 3.32E−06 | −29.86904762 | Hypo-methylated | PCa\|NT DMRs |
| chr19 | 56608157 | 56608326 | 0.000974795 | −22.77380952 | Hypo-methylated | PCa\|NT DMRs |
| chr19 | 56608485 | 56609405 | 9.92E−08 | −48.2047619 | Hypo-methylated | PCa\|NT DMRs |
| chr19 | 56612917 | 56614685 | 0.000207488 | 21.35548654 | Hyper-methylated | PCa\|NT DMRs |
| chr19 | 56617223 | 56618105 | 4.08E−06 | 29.52321429 | Hyper-methylated | PCa\|NT DMRs |
| chr19 | 57356004 | 57356483 | 0.007628253 | −29.44897959 | Hypo-methylated | PCa\|NT DMRs |
| chr19 | 57375887 | 57376129 | 4.43E−05 | 23.47069597 | Hyper-methylated | PCa\|NT DMRs |
| chr19 | 58314937 | 58325339 | 0.000731086 | −26.4984127 | Hypo-methylated | PCa\|NT DMRs |
| chr19 | 58520847 | 58521755 | 0.000349861 | 28.92 | Hyper-methylated | PCa\|NT DMRs |
| chr19 | 58715677 | 58715746 | 0.000909347 | 25.86507937 | Hyper-methylated | PCa\|NT DMRs |
| chr19 | 58839320 | 58839436 | 0.001115302 | −29.51785714 | Hypo-methylated | PCa\|NT DMRs |
| chr19 | 58860844 | 58865165 | 0.000166027 | −22.52857143 | Hypo-methylated | PCa\|NT DMRs |
| chr19 | 58907941 | 58910708 | 2.07E−08 | 24.83158263 | Hyper-methylated | PCa\|NT DMRs |
| chr19 | 58916382 | 58916576 | 0.004266047 | −26.55584416 | Hypo-methylated | PCa\|NT DMRs |
| chr20 | 290953 | 291152 | 2.85E−05 | 33.84835165 | Hyper-methylated | PCa\|NT DMRs |
| chr20 | 370829 | 371257 | 0.009924186 | −25.04867725 | Hypo-methylated | PCa\|NT DMRs |
| chr20 | 398770 | 401742 | 1.52E−10 | −30.97703081 | Hypo-methylated | PCa\|NT DMRs |
| chr20 | 623281 | 623336 | 0.004095673 | 28.78571429 | Hyper-methylated | PCa\|NT DMRs |
| chr20 | 680838 | 681383 | 0.000170278 | −41.92738095 | Hypo-methylated | PCa\|NT DMRs |
| chr20 | 1783684 | 1784552 | 9.09E−30 | 23.9414966 | Hyper-methylated | PCa\|NT DMRs |
| chr20 | 1876471 | 1877094 | 3.44E−05 | 35.09448622 | Hyper-methylated | PCa\|NT DMRs |
| chr20 | 1893501 | 1893587 | 0.001969641 | −35.88095238 | Hypo-methylated | PCa\|NT DMRs |
| chr20 | 1918171 | 1926404 | 0.001098825 | 20.21288515 | Hyper-methylated | PCa\|NT DMRs |
| chr20 | 2510691 | 2521029 | 0.000138104 | −22.48388278 | Hypo-methylated | PCa\|NT DMRs |
| chr20 | 2655366 | 2667918 | 5.42E−05 | −30.0015873 | Hypo-methylated | PCa\|NT DMRs |
| chr20 | 2668808 | 2670350 | 0.003314163 | 35.17619048 | Hyper-methylated | PCa\|NT DMRs |
| chr20 | 2729998 | 2731561 | 5.89E−06 | 26.17453839 | Hyper-methylated | PCa\|NT DMRs |
| chr20 | 2781180 | 2782256 | 1.02E−06 | 20.14887218 | Hyper-methylated | PCa\|NT DMRs |
| chr20 | 2801591 | 2803966 | 5.73E−13 | 30.34688013 | Hyper-methylated | PCa\|NT DMRs |
| chr20 | 2852740 | 2853092 | 0.00179283 | 26.53679654 | Hyper-methylated | PCa\|NT DMRs |
| chr20 | 3052043 | 3052923 | 9.29E−14 | 34.21098485 | Hyper-methylated | PCa\|NT DMRs |
| chr20 | 3063862 | 3064520 | 0.000161254 | 39.41002506 | Hyper-methylated | PCa\|NT DMRs |
| chr20 | 3073286 | 3075043 | 2.38E−17 | 24.93670412 | Hyper-methylated | PCa\|NT DMRs |
| chr20 | 3218646 | 3219205 | 5.20E−13 | 35.7965368 | Hyper-methylated | PCa\|NT DMRs |
| chr20 | 3220409 | 3220986 | 1.64E−15 | 35.93303571 | Hyper-methylated | PCa\|NT DMRs |
| chr20 | 3732490 | 3733265 | 3.41E−19 | 28.77585513 | Hyper-methylated | PCa\|NT DMRs |
| chr20 | 5046531 | 5046645 | 0.001146289 | 23.01785714 | Hyper-methylated | PCa\|NT DMRs |
| chr20 | 5641563 | 5641760 | 0.005968871 | 39.82857143 | Hyper-methylated | PCa\|NT DMRs |
| chr20 | 6160596 | 6723432 | 8.08E−12 | −32.2335277 | Hypo-methylated | PCa\|NT DMRs |
| chr20 | 6783939 | 7969623 | 1.39E−26 | −35.28099048 | Hypo-methylated | PCa\|NT DMRs |
| chr20 | 10603943 | 10647944 | 1.92E−05 | −26.14761905 | Hypo-methylated | PCa\|NT DMRs |
| chr20 | 11622746 | 11658545 | 0.00064571 | −31.90544218 | Hypo-methylated | PCa\|NT DMRs |
| chr20 | 11690071 | 11835274 | 1.63E−07 | −32.35930736 | Hypo-methylated | PCa\|NT DMRs |
| chr20 | 12020714 | 12873410 | 3.95E−18 | −32.26139194 | Hypo-methylated | PCa\|NT DMRs |
| chr20 | 13828761 | 13927899 | 4.29E−08 | −29.45646259 | Hypo-methylated | PCa\|NT DMRs |
| chr20 | 16106790 | 16287187 | 0.005438982 | −32.06904762 | Hypo-methylated | PCa\|NT DMRs |
| chr20 | 17595189 | 17595700 | 1.78E−05 | −36.75600907 | Hypo-methylated | PCa\|NT DMRs |
| chr20 | 17595971 | 17596423 | 4.89E−05 | 30.46369048 | Hyper-methylated | PCa\|NT DMRs |
| chr20 | 17598252 | 17601120 | 0.003573252 | −24.11734694 | Hypo-methylated | PCa\|NT DMRs |
| chr20 | 17844683 | 17851138 | 0.005513661 | −21.2244898 | Hypo-methylated | PCa\|NT DMRs |
| chr20 | 17936940 | 17937110 | 0.001969641 | 45.26587302 | Hyper-methylated | PCa\|NT DMRs |
| chr20 | 18233839 | 18250127 | 6.98E−05 | −23.68367347 | Hypo-methylated | PCa\|NT DMRs |
| chr20 | 18485311 | 18485580 | 9.30E−06 | −28.17142857 | Hypo-methylated | PCa\|NT DMRs |
| chr20 | 18794791 | 18818686 | 8.00E−06 | −26.81532091 | Hypo-methylated | PCa\|NT DMRs |
| chr20 | 19523960 | 19524213 | 0.001969641 | 35.3968254 | Hyper-methylated | PCa\|NT DMRs |
| chr20 | 19642546 | 19669183 | 0.000836132 | −35.53846154 | Hypo-methylated | PCa\|NT DMRs |
| chr20 | 20344717 | 20348271 | 1.31E−34 | 26.1655754 | Hyper-methylated | PCa\|NT DMRs |
| chr20 | 21390540 | 21442274 | 0.00641359 | −32.02301587 | Hypo-methylated | PCa\|NT DMRs |
| chr20 | 21497910 | 21498880 | 6.67E−10 | 26.23809524 | Hyper-methylated | PCa\|NT DMRs |
| chr20 | 21589178 | 21596682 | 0.001789867 | −39.82539683 | Hypo-methylated | PCa\|NT DMRs |
| chr20 | 21687395 | 21695629 | 6.88E−16 | 24.47631416 | Hyper-methylated | PCa\|NT DMRs |
| chr20 | 22557524 | 22557992 | 2.26E−07 | 21.02063492 | Hyper-methylated | PCa\|NT DMRs |
| chr20 | 23031774 | 23032167 | 0.000481535 | 21.85714286 | Hyper-methylated | PCa\|NT DMRs |
| chr20 | 23066264 | 23067193 | 2.17E−11 | 44.94617605 | Hyper-methylated | PCa\|NT DMRs |
| chr20 | 24318195 | 24415710 | 5.24E−06 | −35.83690476 | Hypo-methylated | PCa\|NT DMRs |
| chr20 | 24637710 | 24751108 | 1.66E−32 | −32.46536797 | Hypo-methylated | PCa\|NT DMRs |
| chr20 | 24767787 | 24852446 | 6.16E−21 | −37.34133885 | Hypo-methylated | PCa\|NT DMRs |
| chr20 | 24859448 | 24873322 | 0.000411983 | −26.20922619 | Hypo-methylated | PCa\|NT DMRs |
| chr20 | 24929954 | 24930149 | 0.000481535 | 39.95873016 | Hyper-methylated | PCa\|NT DMRs |
| chr20 | 25085136 | 25086082 | 0.000260936 | −23.98952381 | Hypo-methylated | PCa\|NT DMRs |
| chr20 | 25128565 | 25129142 | 0.002680483 | 20.00729167 | Hyper-methylated | PCa\|NT DMRs |
| chr20 | 25257915 | 25258763 | 0.009924186 | −28.66666667 | Hypo-methylated | PCa\|NT DMRs |
| chr20 | 25262245 | 25262737 | 2.50E−05 | −31.31972789 | Hypo-methylated | PCa\|NT DMRs |
| chr20 | 25290763 | 25290885 | 0.000117699 | −38.72727273 | Hypo-methylated | PCa\|NT DMRs |
| chr20 | 26205151 | 26205219 | 0.001969641 | −31.52619048 | Hypo-methylated | PCa\|NT DMRs |
| chr20 | 26248196 | 26257114 | 0.001181579 | −35.12244898 | Hypo-methylated | PCa\|NT DMRs |
| chr20 | 29439365 | 29439433 | 0.001969641 | −41.6031746 | Hypo-methylated | PCa\|NT DMRs |
| chr20 | 29516322 | 29517183 | 4.81E−05 | −25.87642857 | Hypo-methylated | PCa\|NT DMRs |
| chr20 | 29596137 | 29596829 | 0.000570576 | −25.31206349 | Hypo-methylated | PCa\|NT DMRs |
| chr20 | 30080173 | 30094556 | 0.005513661 | −24.57954145 | Hypo-methylated | PCa\|NT DMRs |
| chr20 | 30156002 | 30156074 | 0.00576331 | 21.44642857 | Hyper-methylated | PCa\|NT DMRs |
| chr20 | 30193523 | 30193987 | 1.00E−11 | 28.17706767 | Hyper-methylated | PCa\|NT DMRs |
| chr20 | 30420016 | 30422355 | 0.002767976 | −20.62683983 | Hypo-methylated | PCa\|NT DMRs |
| chr20 | 30639262 | 30639427 | 0.000974795 | 25.98690476 | Hyper-methylated | PCa\|NT DMRs |
| chr20 | 30646236 | 30646860 | 0.005968871 | −28.08571429 | Hypo-methylated | PCa\|NT DMRs |
| chr20 | 30681095 | 30695433 | 7.57E−05 | −27.94444444 | Hypo-methylated | PCa\|NT DMRs |
| chr20 | 31062364 | 31063486 | 0.001480644 | 26.39761905 | Hyper-methylated | PCa\|NT DMRs |
| chr20 | 31162344 | 31162702 | 0.000179572 | 25.46845238 | Hyper-methylated | PCa\|NT DMRs |
| chr20 | 31168764 | 31170864 | 6.69E−05 | 28.61986864 | Hyper-methylated | PCa\|NT DMRs |
| chr20 | 31210531 | 31210734 | 6.69E−05 | −37.28285714 | Hypo-methylated | PCa\|NT DMRs |
| chr20 | 31261124 | 31261452 | 5.84E−05 | 55.21428571 | Hyper-methylated | PCa\|NT DMRs |
| chr20 | 32232529 | 32239958 | 1.70E−05 | −41.3047619 | Hypo-methylated | PCa\|NT DMRs |
| chr20 | 32321117 | 32321196 | 0.005674668 | −25.63265306 | Hypo-methylated | PCa\|NT DMRs |
| chr20 | 32450338 | 32450413 | 0.004037929 | −35.06122449 | Hypo-methylated | PCa\|NT DMRs |
| chr20 | 32677589 | 32677897 | 0.000117699 | −54.4025974 | Hypo-methylated | PCa\|NT DMRs |
| chr20 | 32856780 | 32857228 | 1.79E−12 | 39.07809524 | Hyper-methylated | PCa\|NT DMRs |
| chr20 | 32978650 | 32978855 | 0.003622801 | −34.88095238 | Hypo-methylated | PCa\|NT DMRs |
| chr20 | 33146756 | 33147398 | 3.36E−05 | −25.63809524 | Hypo-methylated | PCa\|NT DMRs |
| chr20 | 34187634 | 34189196 | 0.000260936 | 30.16071429 | Hyper-methylated | PCa\|NT DMRs |
| chr20 | 34190058 | 34190493 | 0.000155784 | 35.72380952 | Hyper-methylated | PCa\|NT DMRs |
| chr20 | 34316555 | 34329144 | 0.007383952 | −27.06603175 | Hypo-methylated | PCa\|NT DMRs |
| chr20 | 34742966 | 34743093 | 0.001115302 | 20.62797619 | Hyper-methylated | PCa\|NT DMRs |
| chr20 | 35089169 | 35089307 | 0.002097193 | −33.83741497 | Hypo-methylated | PCa\|NT DMRs |
| chr20 | 35210565 | 35233597 | 0.000308231 | 29.81166667 | Hyper-methylated | PCa\|NT DMRs |
| chr20 | 36147141 | 36147987 | 3.20E−05 | −35.3744898 | Hypo-methylated | PCa\|NT DMRs |
| chr20 | 36148716 | 36150113 | 9.53E−10 | 27.6804878 | Hyper-methylated | PCa\|NT DMRs |
| chr20 | 36298866 | 36299145 | 0.000457395 | −26.23626374 | Hypo-methylated | PCa\|NT DMRs |
| chr20 | 36526505 | 36526881 | 0.009882246 | −34.50595238 | Hypo-methylated | PCa\|NT DMRs |
| chr20 | 36543025 | 36552056 | 8.76E−09 | 32.9706044 | Hyper-methylated | PCa\|NT DMRs |
| chr20 | 37228418 | 37228532 | 0.000205174 | −47.025 | Hypo-methylated | PCa\|NT DMRs |
| chr20 | 37230413 | 37230564 | 6.42E−05 | 45.14285714 | Hyper-methylated | PCa\|NT DMRs |
| chr20 | 37433373 | 37434397 | 0.000337681 | 25.30914286 | Hyper-methylated | PCa\|NT DMRs |
| chr20 | 37435354 | 37435836 | 1.28E−07 | 40.01133787 | Hyper-methylated | PCa\|NT DMRs |
| chr20 | 37784794 | 37784928 | 0.005968871 | 27.94285714 | Hyper-methylated | PCa\|NT DMRs |
| chr20 | 37897292 | 38042252 | 2.27E−05 | −25.21028571 | Hypo-methylated | PCa\|NT DMRs |
| chr20 | 38181252 | 38263740 | 0.002585277 | −29.61428571 | Hypo-methylated | PCa\|NT DMRs |
| chr20 | 39311309 | 39312812 | 2.09E−20 | 50.318065 | Hyper-methylated | PCa\|NT DMRs |
| chr20 | 39515963 | 39516096 | 0.000205174 | 27.70892857 | Hyper-methylated | PCa\|NT DMRs |
| chr20 | 39526058 | 39579525 | 7.79E−05 | −22.46481481 | Hypo-methylated | PCa\|NT DMRs |
| chr20 | 41573880 | 41598857 | 0.004231599 | −21.91208791 | Hypo-methylated | PCa\|NT DMRs |
| chr20 | 41766958 | 41767191 | 0.005513661 | −34.375 | Hypo-methylated | PCa\|NT DMRs |
| chr20 | 41818750 | 42018283 | 4.80E−10 | −22.2881459 | Hypo-methylated | PCa\|NT DMRs |
| chr20 | 42395188 | 42437622 | 0.002193447 | −30.504329 | Hypo-methylated | PCa\|NT DMRs |
| chr20 | 42697099 | 42701838 | 1.46E−06 | 39.49343186 | Hyper-methylated | PCa\|NT DMRs |
| chr20 | 42815550 | 42816152 | 6.21E−12 | 25.37272727 | Hyper-methylated | PCa\|NT DMRs |
| chr20 | 42875841 | 42876602 | 6.22E−07 | 32.38662132 | Hyper-methylated | PCa\|NT DMRs |
| chr20 | 43161153 | 43161221 | 0.004007303 | −31.83333333 | Hypo-methylated | PCa\|NT DMRs |
| chr20 | 43209284 | 43238439 | 0.008083098 | −20.83333333 | Hypo-methylated | PCa\|NT DMRs |
| chr20 | 43279741 | 43280062 | 2.06E−05 | 39.86360544 | Hyper-methylated | PCa\|NT DMRs |
| chr20 | 43378690 | 43378973 | 7.28E−06 | 29.87433862 | Hyper-methylated | PCa\|NT DMRs |
| chr20 | 43437977 | 43438255 | 6.17E−06 | 40.42857143 | Hyper-methylated | PCa\|NT DMRs |
| chr20 | 43966423 | 43973156 | 0.003032307 | −25.1122449 | Hypo-methylated | PCa\|NT DMRs |
| chr20 | 44001656 | 44001943 | 1.76E−05 | 34.25297619 | Hyper-methylated | PCa\|NT DMRs |
| chr20 | 44035317 | 44037625 | 1.28E−12 | 29.33791887 | Hyper-methylated | PCa\|NT DMRs |
| chr20 | 44518794 | 44519291 | 2.33E−10 | 24.92496392 | Hyper-methylated | PCa\|NT DMRs |
| chr20 | 44521160 | 44521740 | 0.000118733 | 42.11111111 | Hyper-methylated | PCa\|NT DMRs |
| chr20 | 44635825 | 44636206 | 0.001969641 | −38.77380952 | Hypo-methylated | PCa\|NT DMRs |
| chr20 | 44718985 | 44720657 | 0.000585936 | −22.0097619 | Hypo-methylated | PCa\|NT DMRs |
| chr20 | 44728285 | 44728476 | 5.18E−06 | 24.37962963 | Hyper-methylated | PCa\|NT DMRs |
| chr20 | 44746651 | 44747798 | 0.000377317 | 28 | Hyper-methylated | PCa\|NT DMRs |
| chr20 | 44751033 | 44751328 | 0.005968871 | −22.2847619 | Hypo-methylated | PCa\|NT DMRs |
| chr20 | 44791466 | 44791697 | 0.001969641 | −21.98571429 | Hypo-methylated | PCa\|NT DMRs |
| chr20 | 44893698 | 44893857 | 0.00064571 | −44.40816327 | Hypo-methylated | PCa\|NT DMRs |
| chr20 | 45017804 | 45034199 | 0.00064571 | −38.7755102 | Hypo-methylated | PCa\|NT DMRs |
| chr20 | 45269307 | 45269682 | 0.000176046 | −44.17532468 | Hypo-methylated | PCa\|NT DMRs |
| chr20 | 45312695 | 45313001 | 5.19E−07 | −58.54395604 | Hypo-methylated | PCa\|NT DMRs |
| chr20 | 45834419 | 45834555 | 0.005968871 | −39.45714286 | Hypo-methylated | PCa\|NT DMRs |
| chr20 | 46305188 | 46305459 | 0.002097193 | −36.96054422 | Hypo-methylated | PCa\|NT DMRs |
| chr20 | 46369875 | 46391734 | 2.31E−06 | −26.15887446 | Hypo-methylated | PCa\|NT DMRs |
| chr20 | 46429621 | 46635704 | 3.13E−54 | −32.98792668 | Hypo-methylated | PCa\|NT DMRs |
| chr20 | 46648634 | 46687893 | 1.05E−06 | −30.76157635 | Hypo-methylated | PCa\|NT DMRs |
| chr20 | 46700377 | 46839599 | 8.49E−36 | −34.12034286 | Hypo-methylated | PCa\|NT DMRs |
| chr20 | 46925015 | 47004869 | 5.04E−14 | −32.67414966 | Hypo-methylated | PCa\|NT DMRs |
| chr20 | 47025514 | 47105192 | 5.89E−27 | −35.21610942 | Hypo-methylated | PCa\|NT DMRs |
| chr20 | 47128181 | 47189006 | 2.99E−18 | −36.13184524 | Hypo-methylated | PCa\|NT DMRs |
| chr20 | 47239078 | 47241358 | 0.005968871 | 26.76666667 | Hyper-methylated | PCa\|NT DMRs |
| chr20 | 47303481 | 47304240 | 0.001969641 | 29.75 | Hyper-methylated | PCa\|NT DMRs |
| chr20 | 47319660 | 47323677 | 0.005249864 | 24.42857143 | Hyper-methylated | PCa\|NT DMRs |
| chr20 | 47439700 | 47443972 | 0.000773332 | 26.1621118 | Hyper-methylated | PCa\|NT DMRs |
| chr20 | 47466744 | 47466982 | 0.005513661 | −23.17857143 | Hypo-methylated | PCa\|NT DMRs |
| chr20 | 48124365 | 48153174 | 0.001241245 | −44.25641026 | Hypo-methylated | PCa\|NT DMRs |
| chr20 | 48184195 | 48184488 | 7.87E−05 | 20.32352941 | Hyper-methylated | PCa\|NT DMRs |
| chr20 | 48533081 | 48533691 | 0.005968871 | −38.48571429 | Hypo-methylated | PCa\|NT DMRs |
| chr20 | 48657930 | 48665142 | 0.007776456 | 26.94940476 | Hyper-methylated | PCa\|NT DMRs |
| chr20 | 48889876 | 48900733 | 8.77E−05 | 26.52642089 | Hyper-methylated | PCa\|NT DMRs |
| chr20 | 48959845 | 48965968 | 0.001969641 | 43.05714286 | Hyper-methylated | PCa\|NT DMRs |
| chr20 | 49011198 | 49011586 | 0.008482405 | 26.76666667 | Hyper-methylated | PCa\|NT DMRs |
| chr20 | 49011792 | 49016749 | 0.006410012 | −28.56147186 | Hypo-methylated | PCa\|NT DMRs |
| chr20 | 49157889 | 49158302 | 0.001181579 | 45.79931973 | Hyper-methylated | PCa\|NT DMRs |
| chr20 | 49208888 | 49209198 | 0.000231104 | −59.88571429 | Hypo-methylated | PCa\|NT DMRs |
| chr20 | 49212739 | 49212793 | 0.004095673 | −43.92857143 | Hypo-methylated | PCa\|NT DMRs |
| chr20 | 49214117 | 49214325 | 0.00103773 | −28.76269841 | Hypo-methylated | PCa\|NT DMRs |
| chr20 | 49307216 | 49308083 | 3.61E−10 | 37.31402116 | Hyper-methylated | PCa\|NT DMRs |
| chr20 | 49483120 | 49483476 | 0.00179283 | −20.35714286 | Hypo-methylated | PCa\|NT DMRs |
| chr20 | 49639950 | 49640120 | 0.006410012 | 20.92857143 | Hyper-methylated | PCa\|NT DMRs |
| chr20 | 49958411 | 49958821 | 0.00641359 | −23.12301587 | Hypo-methylated | PCa\|NT DMRs |
| chr20 | 50106194 | 50120864 | 0.000530246 | 29.09291005 | Hyper-methylated | PCa\|NT DMRs |
| chr20 | 50187063 | 50190741 | 0.005949796 | −20.31148459 | Hypo-methylated | PCa\|NT DMRs |
| chr20 | 50352436 | 50352690 | 0.005968871 | −49.14952381 | Hypo-methylated | PCa\|NT DMRs |
| chr20 | 50369136 | 50374046 | 1.97E−05 | −49.42285714 | Hypo-methylated | PCa\|NT DMRs |
| chr20 | 51959856 | 52000535 | 0.002379616 | −35.69761905 | Hypo-methylated | PCa\|NT DMRs |
| chr20 | 52374188 | 52374314 | 0.001969641 | −43.34603175 | Hypo-methylated | PCa\|NT DMRs |
| chr20 | 54205958 | 54261119 | 0.00023371 | −34.86666667 | Hypo-methylated | PCa\|NT DMRs |
| chr20 | 55199769 | 55203849 | 1.48E−38 | 38.81931429 | Hyper-methylated | PCa\|NT DMRs |
| chr20 | 55500054 | 55500462 | 1.51E−09 | 23.76336996 | Hyper-methylated | PCa\|NT DMRs |
| chr20 | 55842022 | 55842072 | 0.005968871 | 36.10952381 | Hyper-methylated | PCa\|NT DMRs |
| chr20 | 55964707 | 55965826 | 1.79E−12 | 45.79126984 | Hyper-methylated | PCa\|NT DMRs |
| chr20 | 55975496 | 55977927 | 2.74E−05 | 25.43452381 | Hyper-methylated | PCa\|NT DMRs |
| chr20 | 55985155 | 55985278 | 0.001969641 | 40.52380952 | Hyper-methylated | PCa\|NT DMRs |
| chr20 | 55993412 | 55993730 | 0.000822388 | −26.53246753 | Hypo-methylated | PCa\|NT DMRs |
| chr20 | 56008091 | 56008227 | 0.004095673 | 47.79206349 | Hyper-methylated | PCa\|NT DMRs |
| chr20 | 56014753 | 56014896 | 0.005968871 | −44.26095238 | Hypo-methylated | PCa\|NT DMRs |
| chr20 | 56016039 | 56017850 | 0.000431954 | −21.74801587 | Hypo-methylated | PCa\|NT DMRs |
| chr20 | 56025773 | 56027162 | 1.12E−06 | 50.72222222 | Hyper-methylated | PCa\|NT DMRs |
| chr20 | 56035256 | 56039183 | 0.000412053 | 21.85833333 | Hyper-methylated | PCa\|NT DMRs |
| chr20 | 56065548 | 56070965 | 0.009807302 | −28.07857143 | Hypo-methylated | PCa\|NT DMRs |
| chr20 | 56076092 | 56076289 | 0.00179283 | −27.28035714 | Hypo-methylated | PCa\|NT DMRs |
| chr20 | 56124020 | 56131899 | 0.000812507 | −24.55714286 | Hypo-methylated | PCa\|NT DMRs |
| chr20 | 56168621 | 56171766 | 0.006207271 | −28.17857143 | Hypo-methylated | PCa\|NT DMRs |
| chr20 | 56193554 | 56196229 | 1.29E−06 | 37.81428571 | Hyper-methylated | PCa\|NT DMRs |
| chr20 | 56227253 | 56245436 | 6.86E−20 | −36.16281385 | Hypo-methylated | PCa\|NT DMRs |
| chr20 | 56286421 | 56293119 | 0.005433534 | −26.84259259 | Hypo-methylated | PCa\|NT DMRs |
| chr20 | 56345569 | 56347883 | 0.000836132 | −35.75132275 | Hypo-methylated | PCa\|NT DMRs |
| chr20 | 56510281 | 56523179 | 0.001637131 | −22.84702381 | Hypo-methylated | PCa\|NT DMRs |
| chr20 | 57042168 | 57045845 | 4.99E−08 | 27.85859788 | Hyper-methylated | PCa\|NT DMRs |
| chr20 | 57091832 | 57098402 | 0.005968871 | −21.48639456 | Hypo-methylated | PCa\|NT DMRs |
| chr20 | 57143331 | 57144511 | 1.42E−05 | −20.5647619 | Hypo-methylated | PCa\|NT DMRs |
| chr20 | 57224696 | 57225494 | 3.72E−13 | 25.94387755 | Hyper-methylated | PCa\|NT DMRs |
| chr20 | 57295545 | 57337922 | 1.44E−06 | −22.60714286 | Hypo-methylated | PCa\|NT DMRs |
| chr20 | 57555770 | 57555897 | 0.005968871 | −39.85714286 | Hypo-methylated | PCa\|NT DMRs |
| chr20 | 57579515 | 57581991 | 0.00013285 | 22.79365079 | Hyper-methylated | PCa\|NT DMRs |
| chr20 | 57583076 | 57583392 | 0.00064571 | 36.39795918 | Hyper-methylated | PCa\|NT DMRs |
| chr20 | 57816356 | 57868338 | 0.000457395 | −33.01391941 | Hypo-methylated | PCa\|NT DMRs |
| chr20 | 58631001 | 58634023 | 1.97E−05 | 49.69142857 | Hyper-methylated | PCa\|NT DMRs |
| chr20 | 58688881 | 58691755 | 0.001471804 | 24.07380952 | Hyper-methylated | PCa\|NT DMRs |
| chr20 | 58797699 | 58822627 | 0.006096657 | −29.21825397 | Hypo-methylated | PCa\|NT DMRs |
| chr20 | 59053191 | 59053293 | 0.001181579 | 27.76190476 | Hyper-methylated | PCa\|NT DMRs |
| chr20 | 59053543 | 59215311 | 4.15E−14 | −31.77423469 | Hypo-methylated | PCa\|NT DMRs |
| chr20 | 60470520 | 60492722 | 1.98E−18 | −24.54558442 | Hypo-methylated | PCa\|NT DMRs |
| chr20 | 60500037 | 60500596 | 0.004236974 | −25.66571429 | Hypo-methylated | PCa\|NT DMRs |
| chr20 | 60578837 | 60578898 | 0.000117699 | −28.31168831 | Hypo-methylated | PCa\|NT DMRs |
| chr20 | 60596434 | 60598869 | 0.002577156 | −27.46582633 | Hypo-methylated | PCa\|NT DMRs |
| chr20 | 60639173 | 60639848 | 1.15E−05 | −27.33503401 | Hypo-methylated | PCa\|NT DMRs |
| chr20 | 60677711 | 60677903 | 0.005513661 | 31.05059524 | Hyper-methylated | PCa\|NT DMRs |
| chr20 | 60754003 | 60754178 | 0.001969641 | −37.11904762 | Hypo-methylated | PCa\|NT DMRs |
| chr20 | 60790144 | 60791275 | 3.03E−08 | −33.56451613 | Hypo-methylated | PCa\|NT DMRs |
| chr20 | 60814232 | 60814900 | 0.00064571 | −27.78646617 | Hypo-methylated | PCa\|NT DMRs |
| chr20 | 60819809 | 60819888 | 0.001969641 | 47.33333333 | Hyper-methylated | PCa\|NT DMRs |
| chr20 | 60821901 | 60822245 | 0.002826041 | −39.30272109 | Hypo-methylated | PCa\|NT DMRs |
| chr20 | 60881105 | 60883387 | 1.10E−07 | −26.35064935 | Hypo-methylated | PCa\|NT DMRs |
| chr20 | 60927828 | 60928238 | 0.001397404 | −37.03015873 | Hypo-methylated | PCa\|NT DMRs |
| chr20 | 60930593 | 60931437 | 0.002193447 | −21.51298701 | Hypo-methylated | PCa\|NT DMRs |
| chr20 | 60931664 | 60932692 | 5.87E−06 | 24.94444444 | Hyper-methylated | PCa\|NT DMRs |
| chr20 | 61001789 | 61003304 | 5.05E−05 | −31.62605042 | Hypo-methylated | PCa\|NT DMRs |
| chr20 | 61022302 | 61022498 | 0.003435258 | −21.36619048 | Hypo-methylated | PCa\|NT DMRs |
| chr20 | 61048321 | 61050272 | 1.22E−08 | 20.2956595 | Hyper-methylated | PCa\|NT DMRs |
| chr20 | 61087268 | 61088289 | 2.42E−06 | 31.43846154 | Hyper-methylated | PCa\|NT DMRs |
| chr20 | 61088820 | 61090463 | 4.11E−07 | −24.78571429 | Hypo-methylated | PCa\|NT DMRs |
| chr20 | 61151391 | 61152143 | 7.59E−08 | −31.51038961 | Hypo-methylated | PCa\|NT DMRs |
| chr20 | 61158418 | 61159538 | 3.63E−06 | 21.17403994 | Hyper-methylated | PCa\|NT DMRs |
| chr20 | 61185361 | 61185456 | 0.000205174 | 36.3452381 | Hyper-methylated | PCa\|NT DMRs |
| chr20 | 61187582 | 61193999 | 1.13E−05 | −26.59501134 | Hypo-methylated | PCa\|NT DMRs |
| chr20 | 61211763 | 61211873 | 0.00179283 | −20.71071429 | Hypo-methylated | PCa\|NT DMRs |
| chr20 | 61306941 | 61309977 | 0.000412171 | 20.35340136 | Hyper-methylated | PCa\|NT DMRs |
| chr20 | 61325062 | 61329699 | 0.000157125 | −21.70952381 | Hypo-methylated | PCa\|NT DMRs |
| chr20 | 61337185 | 61337431 | 0.00179283 | −20.07428571 | Hypo-methylated | PCa\|NT DMRs |
| chr20 | 61354704 | 61354757 | 0.002585277 | −47.23214286 | Hypo-methylated | PCa\|NT DMRs |
| chr20 | 61358624 | 61360594 | 6.84E−05 | 21.73323202 | Hyper-methylated | PCa\|NT DMRs |
| chr20 | 61371408 | 61372595 | 1.60E−10 | 29.69222222 | Hyper-methylated | PCa\|NT DMRs |
| chr20 | 61384301 | 61384611 | 0.001241245 | −21.37830688 | Hypo-methylated | PCa\|NT DMRs |
| chr20 | 61391266 | 61391514 | 0.001115302 | 23.375 | Hyper-methylated | PCa\|NT DMRs |
| chr20 | 61410484 | 61412536 | 0.000339757 | −42.37489177 | Hypo-methylated | PCa\|NT DMRs |
| chr20 | 61416158 | 61420896 | 5.87E−08 | −28.87404762 | Hypo-methylated | PCa\|NT DMRs |
| chr20 | 61446079 | 61447276 | 0.005302622 | −22.65178571 | Hypo-methylated | PCa\|NT DMRs |
| chr20 | 61450407 | 61450684 | 0.000354065 | 32.20634921 | Hyper-methylated | PCa\|NT DMRs |
| chr20 | 61464517 | 61464689 | 0.003809353 | 29.17142857 | Hyper-methylated | PCa\|NT DMRs |
| chr20 | 61507009 | 61507189 | 0.003448944 | −22.93877551 | Hypo-methylated | PCa\|NT DMRs |
| chr20 | 61554825 | 61554911 | 0.005968871 | −40.65238095 | Hypo-methylated | PCa\|NT DMRs |
| chr20 | 61587509 | 61587829 | 0.007560755 | −31.61607143 | Hypo-methylated | PCa\|NT DMRs |
| chr20 | 61605742 | 61608123 | 0.00841938 | −23.63865546 | Hypo-methylated | PCa\|NT DMRs |
| chr20 | 61609402 | 61610243 | 0.000564835 | 29.57095238 | Hyper-methylated | PCa\|NT DMRs |
| chr20 | 61630617 | 61630803 | 0.001969641 | −27.83333333 | Hypo-methylated | PCa\|NT DMRs |
| chr20 | 61715115 | 61716470 | 2.84E−08 | −28.71493506 | Hypo-methylated | PCa\|NT DMRs |
| chr20 | 61718187 | 61718767 | 0.000386426 | −27.43452381 | Hypo-methylated | PCa\|NT DMRs |
| chr20 | 61731416 | 61732480 | 0.000205174 | −24.81845238 | Hypo-methylated | PCa\|NT DMRs |
| chr20 | 61751153 | 61756554 | 5.47E−12 | −27.5767316 | Hypo-methylated | PCa\|NT DMRs |
| chr20 | 61938692 | 61944033 | 2.69E−09 | −26.3552521 | Hypo-methylated | PCa\|NT DMRs |
| chr20 | 62042829 | 62043086 | 0.000909347 | 26.25793651 | Hyper-methylated | PCa\|NT DMRs |
| chr20 | 62072040 | 62073213 | 0.000354065 | 29.32698413 | Hyper-methylated | PCa\|NT DMRs |
| chr20 | 62077932 | 62080130 | 4.13E−06 | −26.20281385 | Hypo-methylated | PCa\|NT DMRs |
| chr20 | 62085930 | 62088655 | 4.79E−07 | −32.80414747 | Hypo-methylated | PCa\|NT DMRs |
| chr20 | 62102548 | 62102987 | 3.60E−08 | 21.12872024 | Hyper-methylated | PCa\|NT DMRs |
| chr20 | 62116444 | 62116597 | 0.00068166 | −32.08869048 | Hypo-methylated | PCa\|NT DMRs |
| chr20 | 62131026 | 62131332 | 0.000494377 | 30.12993197 | Hyper-methylated | PCa\|NT DMRs |
| chr20 | 62164219 | 62164288 | 0.007959104 | 22.36734694 | Hyper-methylated | PCa\|NT DMRs |
| chr20 | 62167618 | 62168604 | 2.22E−11 | −39.24693878 | Hypo-methylated | PCa\|NT DMRs |
| chr20 | 62170689 | 62170931 | 0.000972067 | −33.5 | Hypo-methylated | PCa\|NT DMRs |
| chr20 | 62173166 | 62173764 | 1.60E−10 | −26.5906298 | Hypo-methylated | PCa\|NT DMRs |
| chr20 | 62178809 | 62178860 | 0.00064571 | 26.6047619 | Hyper-methylated | PCa\|NT DMRs |
| chr20 | 62198188 | 62201158 | 4.67E−09 | 30.51285714 | Hyper-methylated | PCa\|NT DMRs |
| chr20 | 62207186 | 62208923 | 0.000205174 | −48.72857143 | Hypo-methylated | PCa\|NT DMRs |
| chr20 | 62220134 | 62222008 | 2.16E−14 | −39.42569444 | Hypo-methylated | PCa\|NT DMRs |
| chr20 | 62244074 | 62244234 | 0.001969641 | 49.40079365 | Hyper-methylated | PCa\|NT DMRs |
| chr20 | 62317724 | 62319022 | 5.76E−08 | 29.07123421 | Hyper-methylated | PCa\|NT DMRs |
| chr20 | 62328034 | 62328557 | 1.55E−08 | 24.49333333 | Hyper-methylated | PCa\|NT DMRs |
| chr20 | 62367007 | 62370138 | 1.35E−08 | 29.8214986 | Hyper-methylated | PCa\|NT DMRs |
| chr20 | 62434267 | 62435863 | 0.00022692 | 29.05714286 | Hyper-methylated | PCa\|NT DMRs |
| chr20 | 62461656 | 62461764 | 0.004095673 | 32.56746032 | Hyper-methylated | PCa\|NT DMRs |
| chr20 | 62486691 | 62489333 | 0.000106371 | −31.72190476 | Hypo-methylated | PCa\|NT DMRs |
| chr20 | 62509019 | 62510880 | 6.22E−07 | 20.55697279 | Hyper-methylated | PCa\|NT DMRs |
| chr20 | 62522309 | 62522518 | 0.005513661 | 34.20068027 | Hyper-methylated | PCa\|NT DMRs |
| chr20 | 62540851 | 62544255 | 0.002585277 | −27.96428571 | Hypo-methylated | PCa\|NT DMRs |
| chr20 | 62570111 | 62570725 | 1.42E−11 | 31.58730159 | Hyper-methylated | PCa\|NT DMRs |
| chr20 | 62572875 | 62573215 | 1.85E−06 | 43.49803922 | Hyper-methylated | PCa\|NT DMRs |
| chr20 | 62683031 | 62684525 | 8.73E−05 | −24.02482993 | Hypo-methylated | PCa\|NT DMRs |
| chr20 | 62685400 | 62689001 | 2.41E−05 | 30.3686067 | Hyper-methylated | PCa\|NT DMRs |
| chr20 | 62711559 | 62712125 | 4.10E−05 | 20.67969925 | Hyper-methylated | PCa\|NT DMRs |
| chr20 | 62730554 | 62730782 | 0.001969641 | −21.84126984 | Hypo-methylated | PCa\|NT DMRs |
| chr20 | 62738880 | 62739172 | 0.001969641 | 23.61190476 | Hyper-methylated | PCa\|NT DMRs |
| chr20 | 62761065 | 62765629 | 0.004236974 | −32.00238095 | Hypo-methylated | PCa\|NT DMRs |
| chr20 | 62795481 | 62808209 | 1.11E−05 | −22.49201681 | Hypo-methylated | PCa\|NT DMRs |
| chr20 | 62826409 | 62831730 | 4.97E−06 | −26.38543417 | Hypo-methylated | PCa\|NT DMRs |
| chr20 | 62917292 | 62917489 | 0.002826041 | −35.06122449 | Hypo-methylated | PCa\|NT DMRs |
| chr21 | 19629016 | 19815859 | 2.29E−09 | −32.24285714 | Hypo-methylated | PCa\|NT DMRs |
| chr21 | 19824840 | 20907871 | 6.58E−22 | −34.35731092 | Hypo-methylated | PCa\|NT DMRs |
| chr21 | 21863082 | 22361964 | 1.30E−15 | −31.69437939 | Hypo-methylated | PCa\|NT DMRs |
| chr21 | 22369363 | 22370241 | 4.02E−05 | 27.66111111 | Hyper-methylated | PCa\|NT DMRs |
| chr21 | 22987602 | 23162740 | 1.21E−07 | −26.45739348 | Hypo-methylated | PCa\|NT DMRs |
| chr21 | 24349698 | 24393877 | 0.00064571 | −28.82444444 | Hypo-methylated | PCa\|NT DMRs |
| chr21 | 24412021 | 25221284 | 1.50E−31 | −32.06731951 | Hypo-methylated | PCa\|NT DMRs |
| chr21 | 25253813 | 25320535 | 2.85E−13 | −32.67464286 | Hypo-methylated | PCa\|NT DMRs |
| chr21 | 26332666 | 26657828 | 3.72E−11 | −28.81083333 | Hypo-methylated | PCa\|NT DMRs |
| chr21 | 26751761 | 26754835 | 0.005968871 | −21.42380952 | Hypo-methylated | PCa\|NT DMRs |
| chr21 | 27959216 | 28215691 | 0.0001882 | −26.74920635 | Hypo-methylated | PCa\|NT DMRs |
| chr21 | 28217974 | 28219166 | 1.46E−14 | 25.17142857 | Hyper-methylated | PCa\|NT DMRs |
| chr21 | 28307119 | 28337820 | 0.000145094 | −27.25873016 | Hypo-methylated | PCa\|NT DMRs |
| chr21 | 28433838 | 28757505 | 5.16E−21 | −34.6985119 | Hypo-methylated | PCa\|NT DMRs |
| chr21 | 32465775 | 32486363 | 0.002400984 | −23.82349206 | Hypo-methylated | PCa\|NT DMRs |
| chr21 | 32707165 | 32707334 | 0.00641359 | −37.01984127 | Hypo-methylated | PCa\|NT DMRs |
| chr21 | 33345189 | 33345244 | 0.001969641 | −60.33333333 | Hypo-methylated | PCa\|NT DMRs |
| chr21 | 33393836 | 33394208 | 5.19E−07 | −52.56776557 | Hypo-methylated | PCa\|NT DMRs |
| chr21 | 33720364 | 33732136 | 0.00068166 | −35.26666667 | Hypo-methylated | PCa\|NT DMRs |
| chr21 | 33871659 | 33871850 | 0.00064571 | −38.80680272 | Hypo-methylated | PCa\|NT DMRs |
| chr21 | 34406061 | 34406715 | 0.000537598 | −25.296 | Hypo-methylated | PCa\|NT DMRs |
| chr21 | 34940520 | 34940655 | 0.00641359 | −36.69444444 | Hypo-methylated | PCa\|NT DMRs |
| chr21 | 34990080 | 34990123 | 0.005968871 | 22.5047619 | Hyper-methylated | PCa\|NT DMRs |
| chr21 | 35467804 | 35507857 | 0.008581782 | 24.38293651 | Hyper-methylated | PCa\|NT DMRs |
| chr21 | 35574923 | 35575053 | 0.000205174 | 50.67261905 | Hyper-methylated | PCa\|NT DMRs |
| chr21 | 35700156 | 35718445 | 3.31E−05 | −24.91540616 | Hypo-methylated | PCa\|NT DMRs |
| chr21 | 35738859 | 35739404 | 0.005968871 | −34.68095238 | Hypo-methylated | PCa\|NT DMRs |
| chr21 | 35831851 | 35832142 | 5.42E−06 | 20.03760263 | Hyper-methylated | PCa\|NT DMRs |
| chr21 | 36877555 | 36894085 | 0.00641359 | −21.04761905 | Hypo-methylated | PCa\|NT DMRs |
| chr21 | 36900928 | 36901247 | 3.50E−05 | 50.6007326 | Hyper-methylated | PCa\|NT DMRs |
| chr21 | 37081019 | 37127526 | 0.005968871 | −29.00952381 | Hypo-methylated | PCa\|NT DMRs |
| chr21 | 37485304 | 37486241 | 0.001007739 | −21.08829365 | Hypo-methylated | PCa\|NT DMRs |
| chr21 | 37618686 | 37618829 | 0.001969641 | −57.83673469 | Hypo-methylated | PCa\|NT DMRs |
| chr21 | 37794069 | 37794558 | 0.00603903 | −37.74867725 | Hypo-methylated | PCa\|NT DMRs |
| chr21 | 37880782 | 37881862 | 0.00064571 | −34.87037037 | Hypo-methylated | PCa\|NT DMRs |
| chr21 | 37914560 | 37915020 | 1.87E−08 | 37.98351648 | Hyper-methylated | PCa\|NT DMRs |
| chr21 | 38117218 | 38117455 | 8.17E−05 | −34.38311688 | Hypo-methylated | PCa\|NT DMRs |
| chr21 | 38300427 | 38301554 | 0.006096657 | −44.07142857 | Hypo-methylated | PCa\|NT DMRs |
| chr21 | 38379654 | 38381223 | 0.000178482 | 23.30357143 | Hyper-methylated | PCa\|NT DMRs |
| chr21 | 38592962 | 38598034 | 7.03E−11 | 28.8087619 | Hyper-methylated | PCa\|NT DMRs |
| chr21 | 38918897 | 38919581 | 1.67E−05 | 29.30634921 | Hyper-methylated | PCa\|NT DMRs |
| chr21 | 39311492 | 39334840 | 0.001969641 | −34.89444444 | Hypo-methylated | PCa\|NT DMRs |
| chr21 | 39755553 | 39755720 | 0.000972067 | −30.44642857 | Hypo-methylated | PCa\|NT DMRs |
| chr21 | 39813857 | 39815354 | 0.000286238 | −21.35531136 | Hypo-methylated | PCa\|NT DMRs |
| chr21 | 39862516 | 39880984 | 0.009807302 | 32.17904762 | Hyper-methylated | PCa\|NT DMRs |
| chr21 | 40033374 | 40033635 | 6.03E−08 | 22.60912698 | Hyper-methylated | PCa\|NT DMRs |
| chr21 | 40176560 | 40176694 | 0.000974795 | 30.18452381 | Hyper-methylated | PCa\|NT DMRs |
| chr21 | 40421101 | 40455576 | 0.000447319 | −39.175 | Hypo-methylated | PCa\|NT DMRs |
| chr21 | 41160266 | 41160520 | 0.007494574 | −36.18367347 | Hypo-methylated | PCa\|NT DMRs |
| chr21 | 41550841 | 41550908 | 0.000117699 | −33.52380952 | Hypo-methylated | PCa\|NT DMRs |
| chr21 | 42485213 | 42485762 | 0.001969641 | 42.78231293 | Hyper-methylated | PCa\|NT DMRs |
| chr21 | 42722029 | 42729919 | 0.000286238 | −21.55494505 | Hypo-methylated | PCa\|NT DMRs |
| chr21 | 42732378 | 42732745 | 0.002585277 | −22.53214286 | Hypo-methylated | PCa\|NT DMRs |
| chr21 | 42908455 | 42916105 | 0.001115302 | −46.51785714 | Hypo-methylated | PCa\|NT DMRs |
| chr21 | 43025822 | 43026059 | 0.000117699 | −51.81818182 | Hypo-methylated | PCa\|NT DMRs |
| chr21 | 43101509 | 43102244 | 1.81E−05 | −48.48673469 | Hypo-methylated | PCa\|NT DMRs |
| chr21 | 43182655 | 43183989 | 9.63E−06 | 36.5 | Hyper-methylated | PCa\|NT DMRs |
| chr21 | 43221381 | 43221433 | 0.005968871 | −33.6 | Hypo-methylated | PCa\|NT DMRs |
| chr21 | 43221581 | 43222615 | 1.67E−05 | 21.27380952 | Hyper-methylated | PCa\|NT DMRs |
| chr21 | 43222808 | 43222975 | 0.000205174 | −28.67857143 | Hypo-methylated | PCa\|NT DMRs |
| chr21 | 43481974 | 43484442 | 2.05E−05 | 51.86190476 | Hyper-methylated | PCa\|NT DMRs |
| chr21 | 43542566 | 43546018 | 0.009781813 | 25.94047619 | Hyper-methylated | PCa\|NT DMRs |
| chr21 | 43575605 | 43575871 | 0.005968871 | 21.17333333 | Hyper-methylated | PCa\|NT DMRs |
| chr21 | 43685949 | 43690295 | 0.000783791 | 29.55272109 | Hyper-methylated | PCa\|NT DMRs |
| chr21 | 43730825 | 43731038 | 0.001969641 | −25.26666667 | Hypo-methylated | PCa\|NT DMRs |
| chr21 | 43784335 | 43786119 | 0.007959104 | 29.7707483 | Hyper-methylated | PCa\|NT DMRs |
| chr21 | 43786285 | 43786998 | 8.17E−05 | −41.04848485 | Hypo-methylated | PCa\|NT DMRs |
| chr21 | 43945113 | 43951322 | 0.005968871 | −22.14935065 | Hypo-methylated | PCa\|NT DMRs |
| chr21 | 44013448 | 44013840 | 8.96E−05 | 20.64246032 | Hyper-methylated | PCa\|NT DMRs |
| chr21 | 44072805 | 44072939 | 0.005968871 | −47.12857143 | Hypo-methylated | PCa\|NT DMRs |
| chr21 | 44074927 | 44076143 | 0.006207271 | −23.35714286 | Hypo-methylated | PCa\|NT DMRs |
| chr21 | 44079903 | 44080174 | 0.00025637 | 24.2952381 | Hyper-methylated | PCa\|NT DMRs |
| chr21 | 44165294 | 44171359 | 0.007560755 | 30.59821429 | Hyper-methylated | PCa\|NT DMRs |
| chr21 | 44222891 | 44227382 | 0.00641359 | 20.62539683 | Hyper-methylated | PCa\|NT DMRs |
| chr21 | 44259715 | 44260132 | 0.001181579 | 25.6 | Hyper-methylated | PCa\|NT DMRs |
| chr21 | 44373759 | 44375977 | 0.000117699 | −55.35844156 | Hypo-methylated | PCa\|NT DMRs |
| chr21 | 44484698 | 44484875 | 0.004947537 | −43.26878307 | Hypo-methylated | PCa\|NT DMRs |
| chr21 | 44594030 | 44594485 | 0.001419801 | −23.93571429 | Hypo-methylated | PCa\|NT DMRs |
| chr21 | 44597135 | 44597305 | 0.001969641 | 38.33333333 | Hyper-methylated | PCa\|NT DMRs |
| chr21 | 44609016 | 44609085 | 0.005968871 | −48.74285714 | Hypo-methylated | PCa\|NT DMRs |
| chr21 | 44743146 | 44743636 | 0.00064571 | −50.84013605 | Hypo-methylated | PCa\|NT DMRs |
| chr21 | 44746073 | 44746162 | 0.004057568 | 51.64285714 | Hyper-methylated | PCa\|NT DMRs |
| chr21 | 44809968 | 44810286 | 0.003448944 | 38.52721088 | Hyper-methylated | PCa\|NT DMRs |
| chr21 | 44818417 | 44820569 | 1.05E−18 | 50.69002976 | Hyper-methylated | PCa\|NT DMRs |
| chr21 | 44847417 | 44848556 | 3.37E−14 | 22.23240166 | Hyper-methylated | PCa\|NT DMRs |
| chr21 | 44876264 | 44876940 | 8.86E−05 | 29.30238095 | Hyper-methylated | PCa\|NT DMRs |
| chr21 | 44883532 | 44883776 | 0.000474737 | −42.01587302 | Hypo-methylated | PCa\|NT DMRs |
| chr21 | 45025937 | 45077944 | 1.55E−07 | 28.30587302 | Hyper-methylated | PCa\|NT DMRs |
| chr21 | 45148429 | 45149333 | 3.43E−06 | 35.31318681 | Hyper-methylated | PCa\|NT DMRs |
| chr21 | 45246278 | 45247123 | 0.001241245 | −29.54761905 | Hypo-methylated | PCa\|NT DMRs |
| chr21 | 45255205 | 45259032 | 0.005855202 | 25.79464286 | Hyper-methylated | PCa\|NT DMRs |
| chr21 | 45280075 | 45280360 | 0.001969641 | −59.13945578 | Hypo-methylated | PCa\|NT DMRs |
| chr21 | 45336782 | 45337210 | 3.64E−09 | 56.56302521 | Hyper-methylated | PCa\|NT DMRs |
| chr21 | 45393172 | 45401133 | 0.007628253 | −39.93877551 | Hypo-methylated | PCa\|NT DMRs |
| chr21 | 45405820 | 45406212 | 0.000206041 | 27.5952381 | Hyper-methylated | PCa\|NT DMRs |
| chr21 | 45507402 | 45507604 | 0.005968871 | 42.94285714 | Hyper-methylated | PCa\|NT DMRs |
| chr21 | 45566682 | 45567111 | 0.00023371 | −45.08937729 | Hypo-methylated | PCa\|NT DMRs |
| chr21 | 45573632 | 45574168 | 0.001969641 | 42.28968254 | Hyper-methylated | PCa\|NT DMRs |
| chr21 | 45574652 | 45579761 | 4.58E−15 | 45.5125 | Hyper-methylated | PCa\|NT DMRs |
| chr21 | 45585868 | 45586048 | 0.001969641 | −44.38095238 | Hypo-methylated | PCa\|NT DMRs |
| chr21 | 45604990 | 45605084 | 0.00064571 | −36.81632653 | Hypo-methylated | PCa\|NT DMRs |
| chr11 | 45622494 | 45622932 | 0.003350175 | 24.19285714 | Hyper-methylated | PCa\|NT DMRs |
| chr21 | 45626482 | 45626525 | 0.005968871 | −43.14285714 | Hypo-methylated | PCa\|NT DMRs |
| chr21 | 45626727 | 45627636 | 0.000135267 | 30.40142857 | Hyper-methylated | PCa\|NT DMRs |
| chr21 | 45662814 | 45663407 | 5.61E−06 | 23.68818342 | Hyper-methylated | PCa\|NT DMRs |
| chr21 | 45681828 | 45684178 | 0.000303038 | −26.4047619 | Hypo-methylated | PCa\|NT DMRs |
| chr21 | 45762023 | 45769886 | 4.11E−08 | −44.96254826 | Hypo-methylated | PCa\|NT DMRs |
| chr21 | 45770980 | 45772291 | 0.002217636 | −23.0235589 | Hypo-methylated | PCa\|NT DMRs |
| chr21 | 45786709 | 45790853 | 6.80E−06 | −24.26535304 | Hypo-methylated | PCa\|NT DMRs |
| chr21 | 45795149 | 45795643 | 8.17E−05 | 36.22294372 | Hyper-methylated | PCa\|NT DMRs |
| chr21 | 45858462 | 45859181 | 4.89E−08 | −40.54372294 | Hypo-methylated | PCa\|NT DMRs |
| chr21 | 45881443 | 45881685 | 1.97E−05 | −25.09 | Hypo-methylated | PCa\|NT DMRs |
| chr21 | 45918970 | 45919589 | 4.96E−06 | −29.25421245 | Hypo-methylated | PCa\|NT DMRs |
| chr21 | 45923111 | 45924362 | 4.95E−07 | 40.74484127 | Hyper-methylated | PCa\|NT DMRs |
| chr21 | 45951621 | 45953661 | 0.001148141 | −21.21611722 | Hypo-methylated | PCa\|NT DMRs |
| chr21 | 46009781 | 46012423 | 0.003122338 | −22.31190476 | Hypo-methylated | PCa\|NT DMRs |
| chr21 | 46048609 | 46059537 | 0.001899053 | −23.45798319 | Hypo-methylated | PCa\|NT DMRs |
| chr21 | 46059964 | 46066758 | 0.00064571 | −32.43265306 | Hypo-methylated | PCa\|NT DMRs |
| chr21 | 46143226 | 46143567 | 9.08E−08 | 32.65324675 | Hyper-methylated | PCa\|NT DMRs |
| chr21 | 46256658 | 46257034 | 0.003229648 | −27.52606516 | Hypo-methylated | PCa\|NT DMRs |
| chr21 | 46331567 | 46334414 | 7.95E−05 | 31.75384615 | Hyper-methylated | PCa\|NT DMRs |
| chr21 | 46385125 | 46388227 | 1.11E−05 | 23.76155633 | Hyper-methylated | PCa\|NT DMRs |
| chr21 | 46400608 | 46400820 | 5.73E−05 | 26.86335404 | Hyper-methylated | PCa\|NT DMRs |
| chr21 | 46438025 | 46438318 | 0.000789852 | 24.82086168 | Hyper-methylated | PCa\|NT DMRs |
| chr21 | 46676098 | 46677446 | 9.05E−08 | 27.9639881 | Hyper-methylated | PCa\|NT DMRs |
| chr21 | 46731925 | 46737119 | 0.002097193 | −24.02721088 | Hypo-methylated | PCa\|NT DMRs |
| chr21 | 46739385 | 46739579 | 0.005968871 | 37.32857143 | Hyper-methylated | PCa\|NT DMRs |
| chr21 | 46777052 | 46777646 | 1.10E−05 | −42.48839286 | Hypo-methylated | PCa\|NT DMRs |
| chr21 | 46851523 | 46854738 | 0.000143553 | −43.31802721 | Hypo-methylated | PCa\|NT DMRs |
| chr21 | 46891483 | 46892074 | 5.84E−05 | −21.31613757 | Hypo-methylated | PCa\|NT DMRs |
| chr21 | 46932035 | 46933559 | 0.001386887 | −27.22579365 | Hypo-methylated | PCa\|NT DMRs |
| chr21 | 46955438 | 46956000 | 4.30E−08 | 41.90289116 | Hyper-methylated | PCa\|NT DMRs |
| chr21 | 46962851 | 46962991 | 2.17E−06 | 22.64511278 | Hyper-methylated | PCa\|NT DMRs |
| chr21 | 47061050 | 47061209 | 1.20E−05 | −28.24126984 | Hypo-methylated | PCa\|NT DMRs |
| chr21 | 47062234 | 47062453 | 2.60E−07 | 21.23577864 | Hyper-methylated | PCa\|NT DMRs |
| chr21 | 47307011 | 47308084 | 9.60E−06 | −37.31714286 | Hypo-methylated | PCa\|NT DMRs |
| chr21 | 47394623 | 47395431 | 2.13E−08 | 34.62135642 | Hyper-methylated | PCa\|NT DMRs |
| chr21 | 47401832 | 47402423 | 1.72E−09 | 26.33780664 | Hyper-methylated | PCa\|NT DMRs |
| chr21 | 47546355 | 47547110 | 3.20E−05 | −28.6547619 | Hypo-methylated | PCa\|NT DMRs |
| chr21 | 47716347 | 47718007 | 1.56E−15 | 35.67684729 | Hyper-methylated | PCa\|NT DMRs |
| chr21 | 47736996 | 47738292 | 0.007560755 | −36.02467532 | Hypo-methylated | PCa\|NT DMRs |
| chr21 | 47789877 | 47794099 | 0.002988739 | 30.46923077 | Hyper-methylated | PCa\|NT DMRs |
| chr21 | 47799824 | 47800045 | 0.005968871 | −37.2 | Hypo-methylated | PCa\|NT DMRs |
| chr21 | 47971611 | 47971863 | 6.90E−06 | 56.4 | Hyper-methylated | PCa\|NT DMRs |
| chr22 | 17024712 | 17041772 | 0.000840726 | −41.84126984 | Hypo-methylated | PCa\|NT DMRs |
| chr22 | 17045516 | 17070765 | 1.27E−14 | −37.345845 | Hypo-methylated | PCa\|NT DMRs |
| chr22 | 17073132 | 17079179 | 1.09E−05 | −25.3485119 | Hypo-methylated | PCa\|NT DMRs |
| chr22 | 17092789 | 17178490 | 0.007560755 | −27.75714286 | Hypo-methylated | PCa\|NT DMRs |
| chr22 | 17602340 | 17602562 | 5.89E−06 | 37.9789916 | Hyper-methylated | PCa\|NT DMRs |
| chr22 | 17640812 | 17640925 | 0.005968871 | −29.27904762 | Hypo-methylated | PCa\|NT DMRs |
| chr22 | 18048750 | 18051695 | 0.000769918 | −29.36813187 | Hypo-methylated | PCa\|NT DMRs |
| chr22 | 18260049 | 18260960 | 0.000112264 | 39.43419913 | Hyper-methylated | PCa\|NT DMRs |
| chr22 | 18267831 | 18268063 | 0.000252289 | −28.55102041 | Hypo-methylated | PCa\|NT DMRs |
| chr22 | 18545792 | 18553990 | 0.001480644 | −32.67142857 | Hypo-methylated | PCa\|NT DMRs |
| chr22 | 18593981 | 18594193 | 0.000339627 | 20.54285714 | Hyper-methylated | PCa\|NT DMRs |
| chr22 | 18894243 | 18894535 | 0.000363526 | 26.00549451 | Hyper-methylated | PCa\|NT DMRs |
| chr22 | 19091036 | 19091103 | 0.000205174 | −44.96428571 | Hypo-methylated | PCa\|NT DMRs |
| chr22 | 19136326 | 19136786 | 3.17E−10 | 26.63162624 | Hyper-methylated | PCa\|NT DMRs |
| chr22 | 19164879 | 19165263 | 0.000339757 | −32.81755952 | Hypo-methylated | PCa\|NT DMRs |
| chr22 | 19567516 | 19567581 | 0.00064571 | 48 | Hyper-methylated | PCa\|NT DMRs |
| chr22 | 19594773 | 19595141 | 0.004947537 | −21.34920635 | Hypo-methylated | PCa\|NT DMRs |
| chr22 | 19674878 | 19677361 | 1.66E−10 | 39.6547619 | Hyper-methylated | PCa\|NT DMRs |
| chr22 | 19760206 | 19760559 | 1.76E−06 | 46.13095238 | Hyper-methylated | PCa\|NT DMRs |
| chr22 | 19861074 | 19861712 | 1.55E−08 | −42.28038095 | Hypo-methylated | PCa\|NT DMRs |
| chr22 | 19880537 | 19881265 | 0.000463506 | −26.37662338 | Hypo-methylated | PCa\|NT DMRs |
| chr22 | 19937121 | 19941344 | 0.00657254 | −25.89977324 | Hypo-methylated | PCa\|NT DMRs |
| chr22 | 19950054 | 19958632 | 0.000249429 | −26.42730159 | Hypo-methylated | PCa\|NT DMRs |
| chr22 | 19958784 | 19959377 | 0.000251429 | 28.23015873 | Hyper-methylated | PCa\|NT DMRs |
| chr22 | 20019658 | 20019744 | 0.004095673 | 43.16666667 | Hyper-methylated | PCa\|NT DMRs |
| chr22 | 20020636 | 20020811 | 0.00641359 | −35.4047619 | Hypo-methylated | PCa\|NT DMRs |
| chr22 | 20103516 | 20104022 | 0.000753232 | −26.73234127 | Hypo-methylated | PCa\|NT DMRs |
| chr22 | 20145260 | 20147763 | 0.001205158 | 28.13877551 | Hyper-methylated | PCa\|NT DMRs |
| chr22 | 20217535 | 20218769 | 0.00064571 | −43.22222222 | Hypo-methylated | PCa\|NT DMRs |
| chr22 | 20225578 | 20225904 | 0.004266047 | 23.41341991 | Hyper-methylated | PCa\|NT DMRs |
| chr22 | 20778079 | 20778698 | 4.64E−07 | 21.42872024 | Hyper-methylated | PCa\|NT DMRs |
| chr22 | 20792364 | 20792820 | 4.54E−10 | 21.10801394 | Hyper-methylated | PCa\|NT DMRs |
| chr22 | 21386522 | 21387059 | 7.24E−10 | 25.69129252 | Hyper-methylated | PCa\|NT DMRs |
| chr22 | 22005751 | 22006325 | 5.51E−07 | 20.14935065 | Hyper-methylated | PCa\|NT DMRs |
| chr22 | 22271664 | 22271971 | 0.005968871 | −42.24285714 | Hypo-methylated | PCa\|NT DMRs |
| chr22 | 22471728 | 22472619 | 0.003290817 | 21.86466165 | Hyper-methylated | PCa\|NT DMRs |
| chr22 | 22771829 | 22823769 | 0.000118596 | −20.80357143 | Hypo-methylated | PCa\|NT DMRs |
| chr22 | 22937393 | 22981516 | 0.000632596 | −22.13419913 | Hypo-methylated | PCa\|NT DMRs |
| chr22 | 23412990 | 23413666 | 5.16E−08 | 27.75220459 | Hyper-methylated | PCa\|NT DMRs |
| chr22 | 23467048 | 23483924 | 0.001789867 | −31.33386243 | Hypo-methylated | PCa\|NT DMRs |
| chr22 | 23505542 | 23505616 | 0.005968871 | −53.6 | Hypo-methylated | PCa\|NT DMRs |
| chr22 | 23793820 | 23799497 | 0.001302261 | 20.31428571 | Hyper-methylated | PCa\|NT DMRs |
| chr22 | 23863086 | 23863855 | 0.002245191 | −22.08035714 | Hypo-methylated | PCa\|NT DMRs |
| chr22 | 24192128 | 24192579 | 0.003541715 | −28.45714286 | Hypo-methylated | PCa\|NT DMRs |
| chr22 | 24199008 | 24199599 | 3.34E−08 | 20.23479853 | Hyper-methylated | PCa\|NT DMRs |
| chr22 | 24366534 | 24366670 | 0.005968871 | 30.23333333 | Hyper-methylated | PCa\|NT DMRs |
| chr22 | 24373619 | 24383325 | 0.006466965 | −26.21234568 | Hypo-methylated | PCa\|NT DMRs |
| chr22 | 24823025 | 24823951 | 0.005438982 | 30.66904762 | Hyper-methylated | PCa\|NT DMRs |
| chr22 | 24984458 | 24984864 | 2.86E−05 | −52.68424908 | Hypo-methylated | PCa\|NT DMRs |
| chr22 | 25814354 | 25817725 | 3.10E−19 | 27.87907801 | Hyper-methylated | PCa\|NT DMRs |
| chr22 | 25960138 | 25960355 | 0.00068166 | 24.425 | Hyper-methylated | PCa\|NT DMRs |
| chr22 | 26293931 | 26444050 | 5.47E−16 | −31.08997114 | Hypo-methylated | PCa\|NT DMRs |
| chr22 | 26446381 | 26538668 | 0.000130438 | −27.69904762 | Hypo-methylated | PCa\|NT DMRs |
| chr22 | 26566765 | 26567014 | 0.001480644 | 25.17142857 | Hyper-methylated | PCa\|NT DMRs |
| chr22 | 26574231 | 26680433 | 2.70E−07 | −33.03064182 | Hypo-methylated | PCa\|NT DMRs |
| chr22 | 26688282 | 26821060 | 9.84E−10 | −21.1946712 | Hypo-methylated | PCa\|NT DMRs |
| chr22 | 26841909 | 26842115 | 0.00025637 | −41.70277778 | Hypo-methylated | PCa\|NT DMRs |
| chr22 | 26848679 | 26848953 | 2.80E−05 | −57.54945055 | Hypo-methylated | PCa\|NT DMRs |
| chr22 | 27010028 | 27035502 | 0.003464153 | −22.33939394 | Hypo-methylated | PCa\|NT DMRs |
| chr22 | 27041156 | 27048415 | 0.004095673 | −54.62301587 | Hypo-methylated | PCa\|NT DMRs |
| chr22 | 27428507 | 27472362 | 6.42E−05 | −30.59408867 | Hypo-methylated | PCa\|NT DMRs |
| chr22 | 27633325 | 27640471 | 0.000642972 | −21.3702381 | Hypo-methylated | PCa\|NT DMRs |
| chr22 | 27674906 | 27695535 | 0.000199792 | −27.91206349 | Hypo-methylated | PCa\|NT DMRs |
| chr22 | 27914582 | 27929933 | 0.005425083 | −21.48015873 | Hypo-methylated | PCa\|NT DMRs |
| chr22 | 28005684 | 28005892 | 0.007560755 | −33.81845238 | Hypo-methylated | PCa\|NT DMRs |
| chr22 | 28063248 | 28067287 | 0.000425664 | 22.76714286 | Hyper-methylated | PCa\|NT DMRs |
| chr22 | 28198165 | 28198532 | 8.77E−12 | 25.11946779 | Hyper-methylated | PCa\|NT DMRs |
| chr22 | 28246057 | 28277629 | 0.005968871 | −53.33714286 | Hypo-methylated | PCa\|NT DMRs |
| chr22 | 29319928 | 29340184 | 0.000297348 | −46.90327381 | Hypo-methylated | PCa\|NT DMRs |
| chr22 | 29541931 | 29542317 | 6.42E−05 | −44.97883598 | Hypo-methylated | PCa\|NT DMRs |
| chr22 | 29604350 | 29615295 | 0.000408406 | 29.63517665 | Hyper-methylated | PCa\|NT DMRs |
| chr22 | 29698942 | 29699231 | 0.005968871 | −47.31428571 | Hypo-methylated | PCa\|NT DMRs |
| chr22 | 29709628 | 29710115 | 2.40E−09 | 27.17895545 | Hyper-methylated | PCa\|NT DMRs |
| chr22 | 29711829 | 29711970 | 0.004947537 | 24.78306878 | Hyper-methylated | PCa\|NT DMRs |
| chr22 | 29773939 | 29774268 | 0.005968871 | −49.60571429 | Hypo-methylated | PCa\|NT DMRs |
| chr22 | 29885209 | 29886484 | 0.005513661 | 32.76785714 | Hyper-methylated | PCa\|NT DMRs |
| chr22 | 30476067 | 30476433 | 7.95E−13 | 40.14987469 | Hyper-methylated | PCa\|NT DMRs |
| chr22 | 30643018 | 30644081 | 0.007239577 | 24.47301587 | Hyper-methylated | PCa\|NT DMRs |
| chr22 | 30659564 | 30659983 | 1.09E−07 | 31.14897959 | Hyper-methylated | PCa\|NT DMRs |
| chr22 | 30660788 | 30667250 | 4.22E−09 | 44.87155388 | Hyper-methylated | PCa\|NT DMRs |
| chr22 | 30671142 | 30679171 | 1.27E−05 | 29.24024578 | Hyper-methylated | PCa\|NT DMRs |
| chr22 | 30795428 | 30813744 | 7.98E−06 | −22.57275132 | Hypo-methylated | PCa\|NT DMRs |
| chr22 | 31268306 | 31268594 | 0.00064571 | −55.6 | Hypo-methylated | PCa\|NT DMRs |
| chr22 | 31432771 | 31435801 | 0.006589535 | −33.68809524 | Hypo-methylated | PCa\|NT DMRs |
| chr22 | 31480934 | 31481864 | 8.16E−16 | 23.01428571 | Hyper-methylated | PCa\|NT DMRs |
| chr22 | 31744186 | 31746971 | 0.000205174 | −22.33333333 | Hypo-methylated | PCa\|NT DMRs |
| chr22 | 32601601 | 32602722 | 1.13E−06 | −21.96071429 | Hypo-methylated | PCa\|NT DMRs |
| chr22 | 32928683 | 32930418 | 0.00025637 | −31.90396825 | Hypo-methylated | PCa\|NT DMRs |
| chr22 | 32948156 | 32978079 | 3.20E−06 | −26.59389499 | Hypo-methylated | PCa\|NT DMRs |
| chr22 | 32983624 | 32999221 | 3.44E−06 | −26.01041667 | Hypo-methylated | PCa\|NT DMRs |
| chr22 | 33200580 | 33200931 | 0.003448944 | −21.62585034 | Hypo-methylated | PCa\|NT DMRs |
| chr22 | 33216478 | 33245548 | 1.96E−06 | −34.98988095 | Hypo-methylated | PCa\|NT DMRs |
| chr22 | 33305645 | 33323343 | 0.00068166 | −32.6875 | Hypo-methylated | PCa\|NT DMRs |
| chr22 | 33353376 | 33353553 | 0.005968871 | −33.72380952 | Hypo-methylated | PCa\|NT DMRs |
| chr22 | 35021919 | 35090807 | 0.00010579 | −21.97418546 | Hypo-methylated | PCa\|NT DMRs |
| chr22 | 35711812 | 35712080 | 5.19E−07 | −50.9007326 | Hypo-methylated | PCa\|NT DMRs |
| chr22 | 35748539 | 35749528 | 0.006225823 | 24.31385281 | Hyper-methylated | PCa\|NT DMRs |
| chr22 | 35827034 | 35827315 | 0.001241245 | −36.87830688 | Hypo-methylated | PCa\|NT DMRs |
| chr22 | 36123779 | 36123826 | 0.003622801 | −25.45238095 | Hypo-methylated | PCa\|NT DMRs |
| chr22 | 36519405 | 36519925 | 0.005023204 | −37.1672619 | Hypo-methylated | PCa\|NT DMRs |
| chr22 | 36719573 | 36724256 | 0.003913431 | −23.65019841 | Hypo-methylated | PCa\|NT DMRs |
| chr22 | 36769355 | 36775943 | 0.00921822 | 27.18521303 | Hyper-methylated | PCa\|NT DMRs |
| chr22 | 36855559 | 36855813 | 0.00064571 | −49.3462585 | Hypo-methylated | PCa\|NT DMRs |
| chr22 | 36856901 | 36860324 | 0.001969641 | 27.24047619 | Hyper-methylated | PCa\|NT DMRs |
| chr22 | 36861148 | 36861755 | 2.62E−08 | −43.34433498 | Hypo-methylated | PCa\|NT DMRs |
| chr22 | 36864139 | 36864391 | 0.001969641 | 51.0452381 | Hyper-methylated | PCa\|NT DMRs |
| chr22 | 36973128 | 37002514 | 0.001053932 | −20.11450216 | Hypo-methylated | PCa\|NT DMRs |
| chr22 | 37249941 | 37252152 | 0.00064571 | −42.75510204 | Hypo-methylated | PCa\|NT DMRs |
| chr22 | 37465018 | 37465223 | 1.19E−09 | 22.63809524 | Hyper-methylated | PCa\|NT DMRs |
| chr22 | 37466349 | 37471215 | 7.34E−05 | −32.62380952 | Hypo-methylated | PCa\|NT DMRs |
| chr22 | 37482336 | 37483559 | 0.001451132 | −33.4 | Hypo-methylated | PCa\|NT DMRs |
| chr22 | 37523944 | 37528414 | 2.77E−05 | −20.58095238 | Hypo-methylated | PCa\|NT DMRs |
| chr22 | 37637434 | 37641596 | 3.46E−06 | 36.20164835 | Hyper-methylated | PCa\|NT DMRs |
| chr22 | 37694128 | 37697092 | 0.00179283 | 45.65892857 | Hyper-methylated | PCa\|NT DMRs |
| chr22 | 37705340 | 37707327 | 0.001969641 | 39.90873016 | Hyper-methylated | PCa\|NT DMRs |
| chr22 | 37720720 | 37721514 | 1.12E−14 | 30.94480078 | Hyper-methylated | PCa\|NT DMRs |
| chr22 | 37742108 | 37742350 | 6.42E−05 | −44.3968254 | Hypo-methylated | PCa\|NT DMRs |
| chr22 | 37796358 | 37798484 | 0.000117699 | −41.04761905 | Hypo-methylated | PCa\|NT DMRs |
| chr22 | 37880096 | 37882206 | 5.60E−19 | 53.38805488 | Hyper-methylated | PCa\|NT DMRs |
| chr22 | 37897473 | 37898146 | 0.002034353 | −35.47959184 | Hypo-methylated | PCa\|NT DMRs |
| chr22 | 37908752 | 37909243 | 0.003290817 | −55.38392857 | Hypo-methylated | PCa\|NT DMRs |
| chr22 | 37922526 | 37937792 | 0.00248539 | −21.93095238 | Hypo-methylated | PCa\|NT DMRs |
| chr22 | 37942798 | 37943189 | 0.001391421 | −23.31428571 | Hypo-methylated | PCa\|NT DMRs |
| chr22 | 37961882 | 37962646 | 2.24E−06 | 38.91707718 | Hyper-methylated | PCa\|NT DMRs |
| chr22 | 38036101 | 38037250 | 0.004496757 | 22.58646617 | Hyper-methylated | PCa\|NT DMRs |
| chr22 | 38104589 | 38104800 | 6.42E−05 | 37.70740741 | Hyper-methylated | PCa\|NT DMRs |
| chr22 | 38130443 | 38131296 | 0.005513661 | −34.30136054 | Hypo-methylated | PCa\|NT DMRs |
| chr22 | 38221127 | 38221213 | 0.00023371 | 22.08571429 | Hyper-methylated | PCa\|NT DMRs |
| chr22 | 38434955 | 38435760 | 4.11E−06 | −26.39417989 | Hypo-methylated | PCa\|NT DMRs |
| chr22 | 38465060 | 38466279 | 0.001076343 | −28.59090909 | Hypo-methylated | PCa\|NT DMRs |
| chr22 | 38610287 | 38610954 | 0.000916248 | 21.78042328 | Hyper-methylated | PCa\|NT DMRs |
| chr22 | 38687381 | 38687683 | 0.001789867 | −25.87142857 | Hypo-methylated | PCa\|NT DMRs |
| chr22 | 38695196 | 38696915 | 1.00E−09 | −26.60842491 | Hypo-methylated | PCa\|NT DMRs |
| chr22 | 38809164 | 38809414 | 5.84E−05 | −43.76984127 | Hypo-methylated | PCa\|NT DMRs |
| chr22 | 38848522 | 38850791 | 0.001775064 | 29.30793651 | Hyper-methylated | PCa\|NT DMRs |
| chr22 | 39173212 | 39173562 | 3.58E−07 | −21.75124717 | Hypo-methylated | PCa\|NT DMRs |
| chr22 | 39257118 | 39261056 | 0.002218812 | −20.09047619 | Hypo-methylated | PCa\|NT DMRs |
| chr22 | 39395611 | 39401513 | 0.000205174 | −20.39404762 | Hypo-methylated | PCa\|NT DMRs |
| chr22 | 39416764 | 39417804 | 0.00179283 | 37.69345238 | Hyper-methylated | PCa\|NT DMRs |
| chr22 | 39540123 | 39540675 | 2.03E−05 | 27.49583333 | Hyper-methylated | PCa\|NT DMRs |
| chr22 | 39572270 | 39572680 | 0.00046742 | −57.4004329 | Hypo-methylated | PCa\|NT DMRs |
| chr22 | 39584970 | 39599577 | 0.001969641 | −22.82571429 | Hypo-methylated | PCa\|NT DMRs |
| chr22 | 39650960 | 39651432 | 0.001224841 | 31.45238095 | Hyper-methylated | PCa\|NT DMRs |
| chr22 | 39686867 | 39687020 | 0.00064571 | −29.91005291 | Hypo-methylated | PCa\|NT DMRs |
| chr22 | 39688750 | 39690040 | 0.00068166 | −26.48412698 | Hypo-methylated | PCa\|NT DMRs |
| chr22 | 39693649 | 39696527 | 0.000452947 | 27.33730159 | Hyper-methylated | PCa\|NT DMRs |
| chr22 | 39712694 | 39714432 | 1.02E−05 | −28.39214286 | Hypo-methylated | PCa\|NT DMRs |
| chr22 | 39852622 | 39852965 | 0.000371379 | −26.3022409 | Hypo-methylated | PCa\|NT DMRs |
| chr22 | 39919480 | 39925038 | 7.74E−06 | −38.19805195 | Hypo-methylated | PCa\|NT DMRs |
| chr22 | 39977457 | 39984289 | 0.000205174 | −23.47321429 | Hypo-methylated | PCa\|NT DMRs |
| chr22 | 40059727 | 40060150 | 4.84E−06 | −32.50526316 | Hypo-methylated | PCa\|NT DMRs |
| chr22 | 40081350 | 40083093 | 4.33E−11 | 25.52153846 | Hyper-methylated | PCa\|NT DMRs |
| chr22 | 40774554 | 40800716 | 4.37E−05 | 29.37993197 | Hyper-methylated | PCa\|NT DMRs |
| chr22 | 40800876 | 40800984 | 0.001969641 | −22.81746032 | Hypo-methylated | PCa\|NT DMRs |
| chr22 | 41047920 | 41048830 | 2.45E−07 | −30.92019704 | Hypo-methylated | PCa\|NT DMRs |
| chr22 | 41776426 | 41777140 | 1.44E−05 | −38.8531746 | Hypo-methylated | PCa\|NT DMRs |
| chr22 | 41808582 | 41809115 | 0.002105149 | 27.13047619 | Hyper-methylated | PCa\|NT DMRs |
| chr22 | 42063193 | 42071305 | 0.000354065 | −32.02486772 | Hypo-methylated | PCa\|NT DMRs |
| chr22 | 42195954 | 42196351 | 0.000142041 | 26.22435897 | Hyper-methylated | PCa\|NT DMRs |
| chr22 | 42305466 | 42306355 | 6.15E−09 | 24.93557423 | Hyper-methylated | PCa\|NT DMRs |
| chr22 | 42721020 | 42724987 | 0.000457395 | 39.86556777 | Hyper-methylated | PCa\|NT DMRs |
| chr22 | 42832817 | 42833445 | 0.00967147 | −25.51428571 | Hypo-methylated | PCa\|NT DMRs |
| chr22 | 42896908 | 42912274 | 0.006220956 | −21.90714286 | Hypo-methylated | PCa\|NT DMRs |
| chr22 | 43089582 | 43089699 | 0.002437742 | 41.60846561 | Hyper-methylated | PCa\|NT DMRs |
| chr22 | 43165992 | 43167431 | 1.55E−05 | −38.05300207 | Hypo-methylated | PCa\|NT DMRs |
| chr22 | 43264804 | 43264940 | 0.005968871 | −39.24 | Hypo-methylated | PCa\|NT DMRs |
| chr22 | 43524163 | 43524623 | 0.001039378 | 29.72161172 | Hyper-methylated | PCa\|NT DMRs |
| chr22 | 43543732 | 43545804 | 0.001577531 | 38.71941392 | Hyper-methylated | PCa\|NT DMRs |
| chr22 | 43570168 | 43572461 | 0.001004629 | −21.95069124 | Hypo-methylated | PCa\|NT DMRs |
| chr22 | 43582083 | 43582320 | 1.97E−05 | −36.96285714 | Hypo-methylated | PCa\|NT DMRs |
| chr22 | 43593647 | 43595250 | 0.002217888 | −28.08974359 | Hypo-methylated | PCa\|NT DMRs |
| chr22 | 43652842 | 43653384 | 0.000836132 | 42.08201058 | Hyper-methylated | PCa\|NT DMRs |
| chr22 | 43659512 | 43660959 | 7.91E−07 | 39.90123457 | Hyper-methylated | PCa\|NT DMRs |
| chr22 | 43673779 | 43674170 | 0.00064571 | −31.6447619 | Hypo-methylated | PCa\|NT DMRs |
| chr22 | 43688189 | 43689498 | 0.000205174 | −48.96428571 | Hypo-methylated | PCa\|NT DMRs |
| chr22 | 43693489 | 43694062 | 6.42E−05 | −57.01058201 | Hypo-methylated | PCa\|NT DMRs |
| chr22 | 43807305 | 43807614 | 8.06E−09 | 27.84505495 | Hyper-methylated | PCa\|NT DMRs |
| chr22 | 43820980 | 43822229 | 0.00049771 | −25.09166667 | Hypo-methylated | PCa\|NT DMRs |
| chr22 | 44130551 | 44130859 | 0.005968871 | −60.86666667 | Hypo-methylated | PCa\|NT DMRs |
| chr22 | 44220726 | 44227169 | 0.00013778 | −36.04139194 | Hypo-methylated | PCa\|NT DMRs |
| chr22 | 44391797 | 44391940 | 1.97E−05 | 46.4452381 | Hyper-methylated | PCa\|NT DMRs |
| chr22 | 44421022 | 44421293 | 0.00068166 | 20.35059524 | Hyper-methylated | PCa\|NT DMRs |
| chr22 | 44422163 | 44435609 | 0.001881239 | 23.6154195 | Hyper-methylated | PCa\|NT DMRs |
| chr22 | 44442754 | 44464996 | 0.002847038 | 25.51198686 | Hyper-methylated | PCa\|NT DMRs |
| chr22 | 44494384 | 44495099 | 0.002225326 | 22.08163265 | Hyper-methylated | PCa\|NT DMRs |
| chr22 | 44598763 | 44598834 | 0.001969641 | −40.42857143 | Hypo-methylated | PCa\|NT DMRs |
| chr22 | 44697106 | 44708468 | 1.48E−05 | −21.92795031 | Hypo-methylated | PCa\|NT DMRs |
| chr22 | 44796176 | 44796400 | 0.00603903 | −21.73544974 | Hypo-methylated | PCa\|NT DMRs |
| chr22 | 44807644 | 44807931 | 0.000181485 | −20.49417989 | Hypo-methylated | PCa\|NT DMRs |
| chr22 | 44894632 | 44928841 | 0.001969641 | −32.57936508 | Hypo-methylated | PCa\|NT DMRs |
| chr22 | 44973754 | 44973767 | 0.005968871 | 28.16 | Hyper-methylated | PCa\|NT DMRs |
| chr22 | 45055104 | 45055322 | 0.00064571 | −29.31020408 | Hypo-methylated | PCa\|NT DMRs |
| chr22 | 45124762 | 45126133 | 1.00E−10 | 36.41058201 | Hyper-methylated | PCa\|NT DMRs |
| chr22 | 45149396 | 45154567 | 0.000554791 | −32.76106443 | Hypo-methylated | PCa\|NT DMRs |
| chr22 | 45235453 | 45236728 | 9.29E−05 | 30.76857143 | Hyper-methylated | PCa\|NT DMRs |
| chr22 | 45277187 | 45277773 | 6.49E−05 | −20.12566138 | Hypo-methylated | PCa\|NT DMRs |
| chr22 | 45368388 | 45368994 | 0.007560755 | −21.74464286 | Hypo-methylated | PCa\|NT DMRs |
| chr22 | 45621915 | 45632840 | 1.54E−06 | 25.83042973 | Hyper-methylated | PCa\|NT DMRs |
| chr22 | 45836722 | 45836814 | 0.00641359 | 25.14285714 | Hyper-methylated | PCa\|NT DMRs |
| chr22 | 45898180 | 45898468 | 2.24E−06 | 24.28809524 | Hyper-methylated | PCa\|NT DMRs |
| chr22 | 45914606 | 45915974 | 0.001041871 | −26.01203008 | Hypo-methylated | PCa\|NT DMRs |
| chr22 | 45976965 | 45977103 | 0.001969641 | 29.64285714 | Hyper-methylated | PCa\|NT DMRs |
| chr22 | 46028669 | 46029145 | 7.95E−05 | 29.01025641 | Hyper-methylated | PCa\|NT DMRs |
| chr22 | 46202942 | 46230312 | 0.008482405 | −51.52 | Hypo-methylated | PCa\|NT DMRs |
| chr22 | 46263747 | 46266563 | 0.001090251 | 26.1552381 | Hyper-methylated | PCa\|NT DMRs |
| chr22 | 46292209 | 46292665 | 0.001969641 | −46.06746032 | Hypo-methylated | PCa\|NT DMRs |
| chr22 | 46330631 | 46330781 | 0.001969641 | −38.36111111 | Hypo-methylated | PCa\|NT DMRs |
| chr22 | 46392030 | 46392731 | 5.21E−09 | 34.48130511 | Hyper-methylated | PCa\|NT DMRs |
| chr22 | 46450034 | 46454866 | 0.004216674 | −22.77242525 | Hypo-methylated | PCa\|NT DMRs |
| chr22 | 46457715 | 46466113 | 1.64E−07 | −24.14420929 | Hypo-methylated | PCa\|NT DMRs |
| chr22 | 46683803 | 46684365 | 0.000122686 | 40.04251701 | Hyper-methylated | PCa\|NT DMRs |
| chr22 | 46770769 | 46771303 | 5.40E−06 | −39.10873016 | Hypo-methylated | PCa\|NT DMRs |
| chr22 | 46786662 | 46787115 | 0.00023371 | −51.61190476 | Hypo-methylated | PCa\|NT DMRs |
| chr22 | 46921606 | 46921968 | 1.42E−05 | −50.39795918 | Hypo-methylated | PCa\|NT DMRs |
| chr22 | 46934021 | 46934839 | 1.10E−15 | 38.06646403 | Hyper-methylated | PCa\|NT DMRs |
| chr22 | 46973538 | 46975007 | 0.000374633 | 28.12916667 | Hyper-methylated | PCa\|NT DMRs |
| chr22 | 46982366 | 46983354 | 0.000199043 | 24.5026455 | Hyper-methylated | PCa\|NT DMRs |
| chr22 | 47018235 | 47022351 | 0.001221691 | 29.37585034 | Hyper-methylated | PCa\|NT DMRs |
| chr22 | 47036663 | 47037018 | 9.61E−05 | −23.27731092 | Hypo-methylated | PCa\|NT DMRs |
| chr22 | 47057846 | 47058579 | 5.90E−07 | −34.44071429 | Hypo-methylated | PCa\|NT DMRs |
| chr22 | 47068869 | 47071292 | 3.74E−06 | 33.84873354 | Hyper-methylated | PCa\|NT DMRs |
| chr22 | 47143597 | 47143983 | 0.005968871 | −46.41714286 | Hypo-methylated | PCa\|NT DMRs |
| chr22 | 47170473 | 47170814 | 1.42E−05 | 44.95306122 | Hyper-methylated | PCa\|NT DMRs |
| chr22 | 47242609 | 47242924 | 9.61E−05 | 24.16890756 | Hyper-methylated | PCa\|NT DMRs |
| chr22 | 47465566 | 47465709 | 5.94E−06 | −60.44545455 | Hypo-methylated | PCa\|NT DMRs |
| chr22 | 47550304 | 47550383 | 0.004095673 | −52.78571429 | Hypo-methylated | PCa\|NT DMRs |
| chr22 | 47590058 | 47595190 | 5.02E−05 | −26.57142857 | Hypo-methylated | PCa\|NT DMRs |
| chr22 | 47643374 | 47646813 | 6.22E−05 | −27.61258503 | Hypo-methylated | PCa\|NT DMRs |
| chr22 | 47672954 | 47678313 | 1.21E−06 | −27.70634921 | Hypo-methylated | PCa\|NT DMRs |
| chr22 | 47770840 | 47784735 | 3.98E−06 | −22.65 | Hypo-methylated | PCa\|NT DMRs |
| chr22 | 47786550 | 47788537 | 2.80E−05 | −24.11428571 | Hypo-methylated | PCa\|NT DMRs |
| chr22 | 47797235 | 47797962 | 0.00068166 | −29.8 | Hypo-methylated | PCa\|NT DMRs |
| chr22 | 47945842 | 47960854 | 0.000150872 | −23.26995074 | Hypo-methylated | PCa\|NT DMRs |
| chr22 | 47970957 | 48005598 | 7.49E−08 | −32.65176651 | Hypo-methylated | PCa\|NT DMRs |
| chr22 | 48017883 | 48035016 | 2.47E−10 | −30.74817276 | Hypo-methylated | PCa\|NT DMRs |
| chr22 | 48071275 | 48143398 | 4.61E−19 | −24.40392157 | Hypo-methylated | PCa\|NT DMRs |
| chr22 | 48174192 | 48178836 | 3.66E−05 | −27.67619048 | Hypo-methylated | PCa\|NT DMRs |
| chr22 | 48206445 | 48258257 | 1.04E−08 | −26.15607143 | Hypo-methylated | PCa\|NT DMRs |
| chr22 | 48281451 | 48445323 | 5.89E−25 | −29.92323391 | Hypo-methylated | PCa\|NT DMRs |
| chr22 | 48542623 | 48569409 | 5.01E−06 | −34.77738095 | Hypo-methylated | PCa\|NT DMRs |
| chr22 | 48594441 | 48594664 | 0.005968871 | 25.08571429 | Hyper-methylated | PCa\|NT DMRs |
| chr22 | 48605245 | 48633392 | 2.65E−07 | −22.86575481 | Hypo-methylated | PCa\|NT DMRs |
| chr22 | 48705680 | 48710148 | 0.003464153 | −23.09090909 | Hypo-methylated | PCa\|NT DMRs |
| chr22 | 48722193 | 48730913 | 3.32E−14 | −27.12244898 | Hypo-methylated | PCa\|NT DMRs |
| chr22 | 48788912 | 48790788 | 4.33E−05 | −29.44688645 | Hypo-methylated | PCa\|NT DMRs |
| chr22 | 48827012 | 48852751 | 3.60E−16 | −29.7236715 | Hypo-methylated | PCa\|NT DMRs |
| chr22 | 48907552 | 48928174 | 1.94E−31 | −26.12090942 | Hypo-methylated | PCa\|NT DMRs |
| chr22 | 48932080 | 48933016 | 2.13E−12 | −50.99246032 | Hypo-methylated | PCa\|NT DMRs |
| chr22 | 48934921 | 48935788 | 0.001969641 | −40.86031746 | Hypo-methylated | PCa\|NT DMRs |
| chr22 | 48973192 | 48973360 | 0.000463506 | −26.19264069 | Hypo-methylated | PCa\|NT DMRs |
| chr22 | 48974998 | 48976285 | 2.14E−05 | −30.21929825 | Hypo-methylated | PCa\|NT DMRs |
| chr22 | 48988380 | 48993091 | 1.76E−13 | −26.89069264 | Hypo-methylated | PCa\|NT DMRs |
| chr22 | 48998843 | 48999382 | 1.24E−07 | −26.60952381 | Hypo-methylated | PCa\|NT DMRs |
| chr22 | 49002925 | 49005619 | 1.57E−06 | −29.97321429 | Hypo-methylated | PCa\|NT DMRs |
| chr22 | 49012868 | 49020659 | 1.03E−05 | −36.19917898 | Hypo-methylated | PCa\|NT DMRs |
| chr22 | 49057203 | 49079052 | 7.67E−25 | −24.79627329 | Hypo-methylated | PCa\|NT DMRs |
| chr22 | 49098544 | 49098733 | 0.004947537 | 33.15608466 | Hyper-methylated | PCa\|NT DMRs |
| chr22 | 49114811 | 49115923 | 1.21E−06 | −27.14679089 | Hypo-methylated | PCa\|NT DMRs |
| chr22 | 49200129 | 49203804 | 6.66E−09 | −23.6365353 | Hypo-methylated | PCa\|NT DMRs |
| chr22 | 49368895 | 49369808 | 1.76E−06 | −29.58809524 | Hypo-methylated | PCa\|NT DMRs |
| chr22 | 49413871 | 49442520 | 2.65E−28 | −28.24208683 | Hypo-methylated | PCa\|NT DMRs |
| chr22 | 49447831 | 49456655 | 1.57E−05 | −26.15494505 | Hypo-methylated | PCa\|NT DMRs |
| chr22 | 49477254 | 49485253 | 4.65E−09 | −31.91717172 | Hypo-methylated | PCa\|NT DMRs |
| chr22 | 49485941 | 49489584 | 2.08E−07 | −32.37234432 | Hypo-methylated | PCa\|NT DMRs |
| chr22 | 49490948 | 49549413 | 2.37E−31 | −27.66451701 | Hypo-methylated | PCa\|NT DMRs |
| chr22 | 49993535 | 49994626 | 1.67E−08 | −25.87728938 | Hypo-methylated | PCa\|NT DMRs |
| chr22 | 49994702 | 49994865 | 0.005968871 | 24.67619048 | Hyper-methylated | PCa\|NT DMRs |
| chr22 | 50042460 | 50047785 | 2.44E−07 | −33.31547619 | Hypo-methylated | PCa\|NT DMRs |
| chr22 | 50050819 | 50052205 | 1.42E−05 | 28.34183673 | Hyper-methylated | PCa\|NT DMRs |
| chr22 | 50052375 | 50060289 | 4.80E−11 | −32.49013605 | Hypo-methylated | PCa\|NT DMRs |
| chr22 | 50098234 | 50098524 | 7.78E−07 | 27.59849624 | Hyper-methylated | PCa\|NT DMRs |
| chr22 | 50107193 | 50115324 | 1.19E−09 | −24.95110337 | Hypo-methylated | PCa\|NT DMRs |
| chr22 | 50120593 | 50122105 | 0.0003047 | 28.28095238 | Hyper-methylated | PCa\|NT DMRs |
| chr22 | 50162650 | 50162854 | 0.003577362 | −44.03968254 | Hypo-methylated | PCa\|NT DMRs |
| chr22 | 50164112 | 50165439 | 4.38E−07 | 20.38888889 | Hyper-methylated | PCa\|NT DMRs |
| chr22 | 50201727 | 50208045 | 0.001732398 | 27.22559524 | Hyper-methylated | PCa\|NT DMRs |
| chr22 | 50231946 | 50232926 | 1.47E−06 | 37.90814132 | Hyper-methylated | PCa\|NT DMRs |
| chr22 | 50241737 | 50241917 | 1.87E−06 | 30.88359788 | Hyper-methylated | PCa\|NT DMRs |
| chr22 | 50243185 | 50245708 | 1.98E−05 | 23.50443723 | Hyper-methylated | PCa\|NT DMRs |
| chr22 | 50319194 | 50319747 | 6.94E−06 | 56.84603175 | Hyper-methylated | PCa\|NT DMRs |
| chr22 | 50328391 | 50328954 | 1.01E−05 | 30.859447 | Hyper-methylated | PCa\|NT DMRs |
| chr22 | 50341527 | 50344608 | 1.12E−10 | −38.61342857 | Hypo-methylated | PCa\|NT DMRs |
| chr22 | 50347721 | 50348626 | 0.008581782 | −23.14761905 | Hypo-methylated | PCa\|NT DMRs |
| chr22 | 50356209 | 50358974 | 0.000598356 | −27.1039897 | Hypo-methylated | PCa\|NT DMRs |
| chr22 | 50359001 | 50361044 | 0.000409331 | 28.82352941 | Hyper-methylated | PCa\|NT DMRs |
| chr22 | 50435311 | 50435944 | 0.000392715 | −41.15714286 | Hypo-methylated | PCa\|NT DMRs |
| chr22 | 50444827 | 50447047 | 1.97E−05 | −48.86190476 | Hypo-methylated | PCa\|NT DMRs |
| chr22 | 50450635 | 50453070 | 4.36E−05 | −24.02522046 | Hypo-methylated | PCa\|NT DMRs |
| chr22 | 50465887 | 50466283 | 0.00068166 | −38.57738095 | Hypo-methylated | PCa\|NT DMRs |
| chr22 | 50472321 | 50483852 | 4.42E−08 | 32.18455026 | Hyper-methylated | PCa\|NT DMRs |
| chr22 | 50492324 | 50492421 | 0.001969641 | −26.6984127 | Hypo-methylated | PCa\|NT DMRs |
| chr22 | 50494218 | 50497649 | 4.47E−10 | 21.6475469 | Hyper-methylated | PCa\|NT DMRs |
| chr22 | 50499344 | 50501125 | 7.21E−06 | 31.93537415 | Hyper-methylated | PCa\|NT DMRs |
| chr22 | 50507018 | 50507076 | 0.00068166 | −27.96428571 | Hypo-methylated | PCa\|NT DMRs |
| chr22 | 50522294 | 50528562 | 3.55E−06 | 22.92857143 | Hyper-methylated | PCa\|NT DMRs |
| chr22 | 50550439 | 50551233 | 0.005513661 | 36.83035714 | Hyper-methylated | PCa\|NT DMRs |
| chr22 | 50586613 | 50588185 | 0.004649531 | 22.81772487 | Hyper-methylated | PCa\|NT DMRs |
| chr22 | 50607730 | 50608034 | 6.54E−05 | 27.2464986 | Hyper-methylated | PCa\|NT DMRs |
| chr22 | 50640514 | 50640915 | 0.007679542 | −23.97959184 | Hypo-methylated | PCa\|NT DMRs |
| chr22 | 50689963 | 50690499 | 1.15E−05 | 25.39809524 | Hyper-methylated | PCa\|NT DMRs |
| chr22 | 50720245 | 50722032 | 4.98E−11 | −38.76097561 | Hypo-methylated | PCa\|NT DMRs |
| chr22 | 50879384 | 50879642 | 0.002291701 | −24.25595238 | Hypo-methylated | PCa\|NT DMRs |
| chr22 | 50920427 | 50925202 | 1.88E−05 | −25.15691057 | Hypo-methylated | PCa\|NT DMRs |
| chr22 | 50965219 | 50965614 | 0.000396159 | 41.15428571 | Hyper-methylated | PCa\|NT DMRs |
| chr22 | 50975834 | 50981199 | 0.007679542 | 39.06349206 | Hyper-methylated | PCa\|NT DMRs |
| chr22 | 51002050 | 51010074 | 0.002902476 | −28.38612836 | Hypo-methylated | PCa\|NT DMRs |
| chr22 | 51039741 | 51044178 | 1.47E−18 | 28.0995873 | Hyper-methylated | PCa\|NT DMRs |
| chr22 | 51110120 | 51112461 | 2.82E−07 | 36.80444444 | Hyper-methylated | PCa\|NT DMRs |
| chr22 | 51135020 | 51136679 | 1.43E−17 | −49.90679012 | Hypo-methylated | PCa\|NT DMRs |
| chr22 | 51142482 | 51143093 | 9.98E−06 | 20.38408163 | Hyper-methylated | PCa\|NT DMRs |
| chrX | 3419738 | 3467842 | 1.33E−05 | −34.39375 | Hypo-methylated | PCa\|NT DMRs |
| chrX | 3622147 | 3630620 | 0.008255277 | 23.07956349 | Hyper-methylated | PCa\|NT DMRs |
| chrX | 3674948 | 3719664 | 0.001129594 | −30.88928571 | Hypo-methylated | PCa\|NT DMRs |
| chrX | 3857943 | 4198134 | 5.09E−11 | −37.01630592 | Hypo-methylated | PCa\|NT DMRs |
| chrX | 6196153 | 6532728 | 7.74E−13 | −30.06666667 | Hypo-methylated | PCa\|NT DMRs |
| chrX | 9307696 | 9309231 | 0.001451132 | 44.29365079 | Hyper-methylated | PCa\|NT DMRs |
| chrX | 9848579 | 9848638 | 0.001969641 | −46.07142857 | Hypo-methylated | PCa\|NT DMRs |
| chrX | 10126409 | 10139038 | 0.001243418 | 23.7547619 | Hyper-methylated | PCa\|NT DMRs |
| chrX | 10141439 | 10141658 | 0.003314163 | −20.95952381 | Hypo-methylated | PCa\|NT DMRs |
| chrX | 11577770 | 11639205 | 0.007560755 | −27.27857143 | Hypo-methylated | PCa\|NT DMRs |
| chrX | 11682516 | 11682732 | 0.000260826 | 23.39583333 | Hyper-methylated | PCa\|NT DMRs |
| chrX | 12982947 | 12992733 | 0.003919956 | 29.58795518 | Hyper-methylated | PCa\|NT DMRs |
| chrX | 15518992 | 15606435 | 0.000564835 | 35.35714286 | Hyper-methylated | PCa\|NT DMRs |
| chrX | 18905449 | 18905522 | 0.00068166 | 28.07440476 | Hyper-methylated | PCa\|NT DMRs |
| chrX | 19765856 | 19776416 | 0.007776456 | 38.18639456 | Hyper-methylated | PCa\|NT DMRs |
| chrX | 20285567 | 20287640 | 6.92E−09 | 34.56615187 | Hyper-methylated | PCa\|NT DMRs |
| chrX | 21674268 | 21677027 | 9.11E−20 | 33.54954128 | Hyper-methylated | PCa\|NT DMRs |
| chrX | 21961090 | 21971275 | 0.00064571 | −46.96802721 | Hypo-methylated | PCa\|NT DMRs |
| chrX | 23353489 | 23353964 | 0.006407159 | 27.23073593 | Hyper-methylated | PCa\|NT DMRs |
| chrX | 23804818 | 23815719 | 0.005968871 | −52.88 | Hypo-methylated | PCa\|NT DMRs |
| chrX | 24412948 | 24413615 | 0.005968871 | −33.10095238 | Hypo-methylated | PCa\|NT DMRs |
| chrX | 24665662 | 24665883 | 0.008482405 | 29.15238095 | Hyper-methylated | PCa\|NT DMRs |
| chrX | 27398486 | 27399915 | 6.42E−05 | −43.56031746 | Hypo-methylated | PCa\|NT DMRs |
| chrX | 27481149 | 27536712 | 0.000205174 | −36.3827381 | Hypo-methylated | PCa\|NT DMRs |
| chrX | 30631381 | 30631486 | 0.002437742 | 30.91481481 | Hyper-methylated | PCa\|NT DMRs |
| chrX | 38080365 | 38080757 | 0.000334554 | 29.94395604 | Hyper-methylated | PCa\|NT DMRs |
| chrX | 38422277 | 38558038 | 0.006225823 | −21.81298701 | Hypo-methylated | PCa\|NT DMRs |
| chrX | 38950001 | 39017430 | 0.002097193 | −30.42517007 | Hypo-methylated | PCa\|NT DMRs |
| chrX | 39021905 | 39021937 | 0.005968871 | 32.1952381 | Hyper-methylated | PCa\|NT DMRs |
| chrX | 39297702 | 39435492 | 2.47E−06 | −21.87305459 | Hypo-methylated | PCa\|NT DMRs |
| chrX | 39615035 | 39615537 | 0.000117699 | −53.91125541 | Hypo-methylated | PCa\|NT DMRs |
| chrX | 39631724 | 39639881 | 0.007959104 | 35.85646259 | Hyper-methylated | PCa\|NT DMRs |
| chrX | 39673244 | 39676247 | 6.42E−05 | −54.96825397 | Hypo-methylated | PCa\|NT DMRs |
| chrX | 39764542 | 39765088 | 1.81E−05 | −22.59489796 | Hypo-methylated | PCa\|NT DMRs |
| chrX | 39871593 | 39872580 | 2.89E−13 | 23.30279503 | Hyper-methylated | PCa\|NT DMRs |
| chrX | 40003285 | 40005082 | 4.38E−05 | 29.24196429 | Hyper-methylated | PCa\|NT DMRs |
| chrX | 40064796 | 40064977 | 0.000339757 | 24.82900433 | Hyper-methylated | PCa\|NT DMRs |
| chrX | 40275150 | 40309312 | 0.00641359 | −30.09365079 | Hypo-methylated | PCa\|NT DMRs |
| chrX | 40401751 | 40401820 | 0.003448944 | −21.40816327 | Hypo-methylated | PCa\|NT DMRs |
| chrX | 40703267 | 40718169 | 0.005513661 | −23.25238095 | Hypo-methylated | PCa\|NT DMRs |
| chrX | 41098538 | 41099478 | 0.003871035 | 30.73511905 | Hyper-methylated | PCa\|NT DMRs |
| chrX | 41343060 | 41343256 | 0.003622801 | 25.54761905 | Hyper-methylated | PCa\|NT DMRs |
| chrX | 43741644 | 43774238 | 7.86E−08 | 26.39295238 | Hyper-methylated | PCa\|NT DMRs |
| chrX | 44203596 | 44203713 | 6.42E−05 | 45.17248677 | Hyper-methylated | PCa\|NT DMRs |
| chrX | 44760004 | 44769593 | 0.00023371 | −22.97380952 | Hypo-methylated | PCa\|NT DMRs |
| chrX | 45060311 | 45060916 | 0.008482405 | 49.11619048 | Hyper-methylated | PCa\|NT DMRs |
| chrX | 46433978 | 46434846 | 2.76E−32 | 24.56271429 | Hyper-methylated | PCa\|NT DMRs |
| chrX | 47199127 | 47204759 | 0.003448944 | −23.91564626 | Hypo-methylated | PCa\|NT DMRs |
| chrX | 47444646 | 47446158 | 1.04E−05 | −22.27819549 | Hypo-methylated | PCa\|NT DMRs |
| chrX | 48324152 | 48325878 | 0.001111028 | −26.19100529 | Hypo-methylated | PCa\|NT DMRs |
| chrX | 48409193 | 48409405 | 0.005968871 | 26.99047619 | Hyper-methylated | PCa\|NT DMRs |
| chrX | 48769661 | 48769849 | 0.001129594 | −28.40674603 | Hypo-methylated | PCa\|NT DMRs |
| chrX | 49013813 | 49014517 | 0.001241245 | −40.72486772 | Hypo-methylated | PCa\|NT DMRs |
| chrX | 49021310 | 49023724 | 1.45E−17 | 47.8535892 | Hyper-methylated | PCa\|NT DMRs |
| chrX | 49105107 | 49105166 | 0.001969641 | −43.67063492 | Hypo-methylated | PCa\|NT DMRs |
| chrX | 51138687 | 51139482 | 3.72E−07 | 32.66917293 | Hyper-methylated | PCa\|NT DMRs |
| chrX | 51380484 | 51437482 | 0.001969641 | −35.72108844 | Hypo-methylated | PCa\|NT DMRs |
| chrX | 51718426 | 51778515 | 5.84E−05 | −41.18690476 | Hypo-methylated | PCa\|NT DMRs |
| chrX | 53460616 | 53460813 | 0.003622801 | −39.95634921 | Hypo-methylated | PCa\|NT DMRs |
| chrX | 53468979 | 53469023 | 0.001969641 | 37.28571429 | Hyper-methylated | PCa\|NT DMRs |
| chrX | 53720736 | 53720938 | 0.001969641 | −35.8531746 | Hypo-methylated | PCa\|NT DMRs |
| chrX | 54068973 | 54069131 | 0.005968871 | −31.41428571 | Hypo-methylated | PCa\|NT DMRs |
| chrX | 54385317 | 54385690 | 6.42E−05 | 42.96825397 | Hyper-methylated | PCa\|NT DMRs |
| chrX | 56790661 | 56790796 | 0.000149331 | −41.2961039 | Hypo-methylated | PCa\|NT DMRs |
| chrX | 63638964 | 63929683 | 0.000117699 | −45.90909091 | Hypo-methylated | PCa\|NT DMRs |
| chrX | 64851067 | 64879510 | 0.000481535 | −23.07142857 | Hypo-methylated | PCa\|NT DMRs |
| chrX | 64887811 | 64888466 | 0.000225685 | 26.51666667 | Hyper-methylated | PCa\|NT DMRs |
| chrX | 67802415 | 67822868 | 0.001850915 | −28.74107143 | Hypo-methylated | PCa\|NT DMRs |
| chrX | 68050207 | 68050396 | 0.000840726 | 29.21534392 | Hyper-methylated | PCa\|NT DMRs |
| chrX | 68344900 | 68348695 | 0.001037738 | 35.24571429 | Hyper-methylated | PCa\|NT DMRs |
| chrX | 68353870 | 68354067 | 0.005968871 | −24.66190476 | Hypo-methylated | PCa\|NT DMRs |
| chrX | 70150692 | 70151166 | 1.04E−07 | 24.64980159 | Hyper-methylated | PCa\|NT DMRs |
| chrX | 70882635 | 70882792 | 0.00064571 | 58.09183673 | Hyper-methylated | PCa\|NT DMRs |
| chrX | 71308129 | 71318356 | 0.000974795 | 40.57440476 | Hyper-methylated | PCa\|NT DMRs |
| chrX | 71524800 | 71525167 | 1.27E−05 | 41.64761905 | Hyper-methylated | PCa\|NT DMRs |
| chrX | 76286504 | 76599326 | 6.69E−05 | −38.93666667 | Hypo-methylated | PCa\|NT DMRs |
| chrX | 76703773 | 76704067 | 1.97E−05 | 47.39761905 | Hyper-methylated | PCa\|NT DMRs |
| chrX | 84111607 | 84150561 | 0.000205174 | −38.50952381 | Hypo-methylated | PCa\|NT DMRs |
| chrX | 84581332 | 84940950 | 9.01E−09 | −35.6447619 | Hypo-methylated | PCa\|NT DMRs |
| chrX | 85415435 | 85975267 | 4.90E−08 | −37.0004329 | Hypo-methylated | PCa\|NT DMRs |
| chrX | 97635798 | 98196647 | 4.11E−14 | −26.20857143 | Hypo-methylated | PCa\|NT DMRs |
| chrX | 98627197 | 98627273 | 0.00064571 | −32.14081633 | Hypo-methylated | PCa\|NT DMRs |
| chrX | 98802699 | 99049297 | 2.59E−08 | −26.62557319 | Hypo-methylated | PCa\|NT DMRs |
| chrX | 99306261 | 99551652 | 7.78E−07 | −32.74636591 | Hypo-methylated | PCa\|NT DMRs |
| chrX | 99662504 | 99663256 | 0.000149336 | 34.26078431 | Hyper-methylated | PCa\|NT DMRs |
| chrX | 99663460 | 99663576 | 1.42E−05 | 22.98809524 | Hyper-methylated | PCa\|NT DMRs |
| chrX | 100545995 | 100546334 | 4.63E−10 | 21.31514042 | Hyper-methylated | PCa\|NT DMRs |
| chrX | 100805372 | 100805790 | 0.00185014 | 33.01768707 | Hyper-methylated | PCa\|NT DMRs |
| chrX | 101429500 | 101440472 | 0.000396159 | −33.19190476 | Hypo-methylated | PCa\|NT DMRs |
| chrX | 101802112 | 101802437 | 0.00179283 | 29.81845238 | Hyper-methylated | PCa\|NT DMRs |
| chrX | 102001147 | 102001558 | 6.58E−10 | 30.90646259 | Hyper-methylated | PCa\|NT DMRs |
| chrX | 103295075 | 103331097 | 2.86E−07 | −40.23537415 | Hypo-methylated | PCa\|NT DMRs |
| chrX | 103977994 | 103978159 | 0.008482405 | −37.78761905 | Hypo-methylated | PCa\|NT DMRs |
| chrX | 105066606 | 105066895 | 0.001969641 | 30.52380952 | Hyper-methylated | PCa\|NT DMRs |
| chrX | 106694438 | 106694844 | 0.000205174 | 45.4375 | Hyper-methylated | PCa\|NT DMRs |
| chrX | 106961830 | 106982201 | 0.002593284 | −29.16292517 | Hypo-methylated | PCa\|NT DMRs |
| chrX | 107887851 | 107978125 | 0.003205074 | 24.8012987 | Hyper-methylated | PCa\|NT DMRs |
| chrX | 108868182 | 108868383 | 4.96E−06 | 27.69413919 | Hyper-methylated | PCa\|NT DMRs |
| chrX | 109058243 | 109158681 | 1.82E−06 | −21.90915751 | Hypo-methylated | PCa\|NT DMRs |
| chrX | 109416507 | 109416654 | 0.005968871 | −55.81428571 | Hypo-methylated | PCa\|NT DMRs |
| chrX | 113817280 | 113817410 | 0.000205174 | 22.67857143 | Hyper-methylated | PCa\|NT DMRs |
| chrX | 114956670 | 114957531 | 0.001969641 | 42.76507937 | Hyper-methylated | PCa\|NT DMRs |
| chrX | 117250111 | 117250305 | 2.25E−05 | 21.02055138 | Hyper-methylated | PCa\|NT DMRs |
| chrX | 118407331 | 118418067 | 0.000191452 | 30.31904762 | Hyper-methylated | PCa\|NT DMRs |
| chrX | 118491924 | 118496048 | 0.00023371 | −23.12333333 | Hypo-methylated | PCa\|NT DMRs |
| chrX | 118653545 | 118666617 | 0.00576331 | 21.94047619 | Hyper-methylated | PCa\|NT DMRs |
| chrX | 118751692 | 118753211 | 0.00064571 | 46.3537415 | Hyper-methylated | PCa\|NT DMRs |
| chrX | 118893071 | 118894740 | 0.00020562 | 20.44 | Hyper-methylated | PCa\|NT DMRs |
| chrX | 119124902 | 119147515 | 1.22E−06 | 27.84315476 | Hyper-methylated | PCa\|NT DMRs |
| chrX | 119404939 | 119458257 | 3.93E−05 | 23.22559524 | Hyper-methylated | PCa\|NT DMRs |
| chrX | 122317388 | 122318642 | 1.32E−08 | 33.21964286 | Hyper-methylated | PCa\|NT DMRs |
| chrX | 123480537 | 123480938 | 2.06E−05 | 22.09557823 | Hyper-methylated | PCa\|NT DMRs |
| chrX | 125430259 | 125679623 | 1.24E−07 | −33.70038095 | Hypo-methylated | PCa\|NT DMRs |
| chrX | 125685949 | 125686094 | 0.001115302 | −30.43928571 | Hypo-methylated | PCa\|NT DMRs |
| chrX | 125807571 | 125864841 | 2.44E−07 | −28.01292517 | Hypo-methylated | PCa\|NT DMRs |
| chrX | 126120088 | 127066119 | 1.14E−11 | −27.85537415 | Hypo-methylated | PCa\|NT DMRs |
| chrX | 128901556 | 128914746 | 0.007683358 | 24.22417582 | Hyper-methylated | PCa\|NT DMRs |
| chrX | 129067840 | 129068339 | 0.000118733 | −23.55132275 | Hypo-methylated | PCa\|NT DMRs |
| chrX | 129113280 | 129114164 | 0.000652011 | 20.30392157 | Hyper-methylated | PCa\|NT DMRs |
| chrX | 130192230 | 130192579 | 4.00E−07 | 28.06738095 | Hyper-methylated | PCa\|NT DMRs |
| chrX | 130202141 | 130217941 | 2.26E−15 | 25.05962733 | Hyper-methylated | PCa\|NT DMRs |
| chrX | 132056790 | 132091788 | 1.60E−19 | 25.74152381 | Hyper-methylated | PCa\|NT DMRs |
| chrX | 133678817 | 133679201 | 2.00E−07 | 22.42142857 | Hyper-methylated | PCa\|NT DMRs |
| chrX | 134124839 | 134125006 | 0.007560755 | 27.00595238 | Hyper-methylated | PCa\|NT DMRs |
| chrX | 134556267 | 134556750 | 0.000172237 | 33.73968254 | Hyper-methylated | PCa\|NT DMRs |
| chrX | 136515424 | 136601303 | 1.76E−06 | −37.90039683 | Hypo-methylated | PCa\|NT DMRs |
| chrX | 142774516 | 142826712 | 4.83E−06 | −34.49107143 | Hypo-methylated | PCa\|NT DMRs |
| chrX | 143313497 | 143314640 | 2.01E−07 | −29.95666667 | Hypo-methylated | PCa\|NT DMRs |
| chrX | 143477542 | 144599111 | 1.68E−17 | −33.58040816 | Hypo-methylated | PCa\|NT DMRs |
| chrX | 144713810 | 144764865 | 0.001241245 | −34.55185185 | Hypo-methylated | PCa\|NT DMRs |
| chrX | 144970738 | 145280934 | 5.78E−09 | −31.74571429 | Hypo-methylated | PCa\|NT DMRs |
| chrX | 145340421 | 145460969 | 0.000205174 | −36.76190476 | Hypo-methylated | PCa\|NT DMRs |
| chrX | 149936797 | 149936898 | 0.005513661 | 28.37346939 | Hyper-methylated | PCa\|NT DMRs |
| chrX | 150134835 | 150140167 | 0.005685503 | −29.69246032 | Hypo-methylated | PCa\|NT DMRs |
| chrX | 150144407 | 150144825 | 2.37E−06 | 33.35914787 | Hyper-methylated | PCa\|NT DMRs |
| chrX | 151143026 | 151143469 | 1.49E−06 | 20.75569358 | Hyper-methylated | PCa\|NT DMRs |
| chrX | 152023843 | 152066309 | 0.001104922 | 29.37868481 | Hyper-methylated | PCa\|NT DMRs |
| chrX | 152753215 | 152754164 | 0.001175696 | −24.5789916 | Hypo-methylated | PCa\|NT DMRs |
| chrX | 152830128 | 152830220 | 0.005968871 | −25.3952381 | Hypo-methylated | PCa\|NT DMRs |
| chrX | 152861426 | 152863799 | 0.000377317 | −42.33630952 | Hypo-methylated | PCa\|NT DMRs |
| chrX | 152876646 | 152878070 | 0.001397404 | −28.41269841 | Hypo-methylated | PCa\|NT DMRs |
| chrX | 152942395 | 152950833 | 0.00024701 | 28.41269841 | Hyper-methylated | PCa\|NT DMRs |
| chrX | 152987722 | 152987887 | 0.005968871 | −36.8 | Hypo-methylated | PCa\|NT DMRs |
| chrX | 153044149 | 153046538 | 0.006173852 | −20.45026455 | Hypo-methylated | PCa\|NT DMRs |
| chrX | 153096570 | 153097110 | 0.005233918 | 21.9997619 | Hyper-methylated | PCa\|NT DMRs |
| chrX | 153135670 | 153136257 | 0.00064571 | −36.1585034 | Hypo-methylated | PCa\|NT DMRs |
| chrX | 153186909 | 153187137 | 1.31E−06 | −30.93439153 | Hypo-methylated | PCa\|NT DMRs |
| chrX | 153210313 | 153210375 | 0.00064571 | 43.59183673 | Hyper-methylated | PCa\|NT DMRs |
| chrX | 153247228 | 153249478 | 1.62E−05 | 49.55380952 | Hyper-methylated | PCa\|NT DMRs |
| chrX | 153265046 | 153265306 | 0.004350823 | −22.02380952 | Hypo-methylated | PCa\|NT DMRs |
| chrX | 153279521 | 153284103 | 0.004900079 | −20.1827381 | Hypo-methylated | PCa\|NT DMRs |
| chrX | 153389029 | 153391568 | 0.000256137 | 21.46071429 | Hyper-methylated | PCa\|NT DMRs |
| chrX | 153402520 | 153402570 | 0.001896858 | 20.67619048 | Hyper-methylated | PCa\|NT DMRs |
| chrX | 153740124 | 153740334 | 0.003093471 | 25.22190476 | Hyper-methylated | PCa\|NT DMRs |
| chrX | 153764058 | 153769683 | 0.000440265 | −35.14077381 | Hypo-methylated | PCa\|NT DMRs |
| chrX | 153885985 | 153886696 | 5.19E−07 | −23.46996337 | Hypo-methylated | PCa\|NT DMRs |
| chrX | 154563531 | 154842269 | 0.002643508 | 36.75119048 | Hyper-methylated | PCa\|NT DMRs |
| chrY | 13463589 | 13464487 | 1.24E−05 | −20.29328231 | Hypo-methylated | PCa\|NT DMRs |
| chrY | 13471045 | 13471610 | 4.38E−05 | −27.97397959 | Hypo-methylated | PCa\|NT DMRs |
| chrY | 13489568 | 13489881 | 0.002879573 | −23.45185185 | Hypo-methylated | PCa\|NT DMRs |
| chrY | 16539933 | 16619252 | 4.62E−05 | −30.31936508 | Hypo-methylated | PCa\|NT DMRs |
| chrY | 18255052 | 18958865 | 3.33E−07 | −31.59047619 | Hypo-methylated | PCa\|NT DMRs |
| chrY | 23111562 | 23112013 | 0.001969641 | −24.47857143 | Hypo-methylated | PCa\|NT DMRs |
| chrY | 23596847 | 23609425 | 0.005118278 | −21.12539683 | Hypo-methylated | PCa\|NT DMRs |
| chr1 | 848739 | 849414 | 0.003348504 | −22.17517869 | Hypo-methylated | NEPC\|CRPC-Adeno DMRs |
| chr1 | 870425 | 871654 | 2.16E−11 | −29.212915 | Hypo-methylated | NEPC\|CRPC-Adeno DMRs |
| chr1 | 984022 | 984429 | 1.97E−07 | −28.24485099 | Hypo-methylated | NEPC\|CRPC-Adeno DMRs |
| chr1 | 986842 | 987058 | 8.37E−09 | −29.75584795 | Hypo-methylated | NEPC\|CRPC-Adeno DMRs |
| chr1 | 1007056 | 1007879 | 4.16E−12 | −21.44892339 | Hypo-methylated | NEPC\|CRPC-Adeno DMRs |
| chr1 | 1133030 | 1133217 | 7.23E−06 | −22.11199295 | Hypo-methylated | NEPC\|CRPC-Adeno DMRs |
| chr1 | 1221147 | 1221832 | 9.13E−07 | −26.34678363 | Hypo-methylated | NEPC\|CRPC-Adeno DMRs |
| chr1 | 1234125 | 1235195 | 0.000307123 | −25.45329067 | Hypo-methylated | NEPC\|CRPC-Adeno DMRs |
| chr1 | 1337686 | 1337736 | 0.001694183 | 30.2672619 | Hyper-methylated | NEPC\|CRPC-Adeno DMRs |
| chr1 | 1353533 | 1355640 | 3.31E−14 | −21.34642492 | Hypo-methylated | NEPC\|CRPC-Adeno DMRs |
| chr1 | 1373372 | 1373611 | 0.004750844 | 24.83164766 | Hyper-methylated | NEPC\|CRPC-Adeno DMRs |
| chr1 | 1397525 | 1398349 | 1.17E−08 | −40.50736315 | Hypo-methylated | NEPC\|CRPC-Adeno DMRs |
| chr1 | 1538487 | 1540691 | 0.000921835 | −20.25311943 | Hypo-methylated | NEPC\|CRPC-Adeno DMRs |
| chr1 | 1567269 | 1567869 | 9.55E−15 | −22.41288022 | Hypo-methylated | NEPC\|CRPC-Adeno DMRs |
| chr1 | 1591256 | 1591305 | 0.005737741 | −20.48202614 | Hypo-methylated | NEPC\|CRPC-Adeno DMRs |
| chr1 | 1879001 | 1887524 | 0.008866725 | −21.28134675 | Hypo-methylated | NEPC\|CRPC-Adeno DMRs |
| chr1 | 1896556 | 1897284 | 1.60E−06 | −23.24402267 | Hypo-methylated | NEPC\|CRPC-Adeno DMRs |
| chr1 | 1940853 | 1946741 | 0.003652661 | −24.92564745 | Hypo-methylated | NEPC\|CRPC-Adeno DMRs |
| chr1 | 1951348 | 1954793 | 0.000169597 | −21.50611664 | Hypo-methylated | NEPC\|CRPC-Adeno DMRs |
| chr1 | 1986028 | 1986276 | 5.27E−10 | 37.39543706 | Hyper-methylated | NEPC\|CRPC-Adeno DMRs |
| chr1 | 2036222 | 2036833 | 2.94E−06 | −44.17522974 | Hypo-methylated | NEPC\|CRPC-Adeno DMRs |
| chr1 | 2110845 | 2111328 | 0.003484081 | −24.96863728 | Hypo-methylated | NEPC\|CRPC-Adeno DMRs |
| chr1 | 2171063 | 2171717 | 0.005899637 | 24.91915094 | Hyper-methylated | NEPC\|CRPC-Adeno DMRs |
| chr1 | 2259879 | 2264759 | 1.48E−09 | −24.76411681 | Hypo-methylated | NEPC\|CRPC-Adeno DMRs |
| chr1 | 2286452 | 2287295 | 6.68E−05 | −20.46129212 | Hypo-methylated | NEPC\|CRPC-Adeno DMRs |
| chr1 | 2307117 | 2308440 | 6.29E−06 | −26.5734858 | Hypo-methylated | NEPC\|CRPC-Adeno DMRs |
| chr1 | 2309867 | 2310085 | 9.68E−10 | −41.35994152 | Hypo-methylated | NEPC\|CRPC-Adeno DMRs |
| chr1 | 2359167 | 2360307 | 1.17E−08 | −21.65369807 | Hypo-methylated | NEPC\|CRPC-Adeno DMRs |
| chr1 | 2362827 | 2366638 | 1.35E−14 | −23.81797005 | Hypo-methylated | NEPC\|CRPC-Adeno DMRs |
| chr1 | 2366802 | 2372772 | 2.35E−08 | −21.960457 | Hypo-methylated | NEPC\|CRPC-Adeno DMRs |
| chr1 | 2379601 | 2384305 | 6.61E−05 | −24.46729379 | Hypo-methylated | NEPC\|CRPC-Adeno DMRs |
| chr1 | 2400991 | 2403836 | 0.001402859 | −27.28723971 | Hypo-methylated | NEPC\|CRPC-Adeno DMRs |
| chr1 | 2472413 | 2472586 | 1.76E−08 | −21.84329825 | Hypo-methylated | NEPC\|CRPC-Adeno DMRs |
| chr1 | 2525361 | 2525577 | 0.008510472 | −22.63391813 | Hypo-methylated | NEPC\|CRPC-Adeno DMRs |
| chr1 | 2706076 | 2706299 | 5.50E−05 | 24.22001762 | Hyper-methylated | NEPC\|CRPC-Adeno DMRs |
| chr1 | 2706411 | 2706476 | 0.000200845 | 27.17 | Hyper-methylated | NEPC\|CRPC-Adeno DMRs |
| chr1 | 2780321 | 2780506 | 0.002201952 | 25.4046841 | Hyper-methylated | NEPC\|CRPC-Adeno DMRs |
| chr1 | 3085851 | 3090628 | 1.17E−14 | −30.39803271 | Hypo-methylated | NEPC\|CRPC-Adeno DMRs |
| chr1 | 3092222 | 3096495 | 4.07E−05 | −23.88819386 | Hypo-methylated | NEPC\|CRPC-Adeno DMRs |
| chr1 | 3375025 | 3377669 | 3.51E−09 | 20.26826225 | Hyper-methylated | NEPC\|CRPC-Adeno DMRs |
| chr1 | 3388196 | 3390288 | 1.55E−05 | 21.22200237 | Hyper-methylated | NEPC\|CRPC-Adeno DMRs |
| chr1 | 3418019 | 3418479 | 0.000287903 | −25.90647773 | Hypo-methylated | NEPC\|CRPC-Adeno DMRs |
| chr1 | 3421599 | 3422096 | 8.77E−08 | −29.39494815 | Hypo-methylated | NEPC\|CRPC-Adeno DMRs |
| chr1 | 3438072 | 3438283 | 5.75E−05 | −28.58918129 | Hypo-methylated | NEPC\|CRPC-Adeno DMRs |
| chr1 | 3459643 | 3460268 | 9.31E−09 | 30.116911 | Hyper-methylated | NEPC\|CRPC-Adeno DMRs |
| chr1 | 3463918 | 3464395 | 0.009822962 | −22.20619681 | Hypo-methylated | NEPC\|CRPC-Adeno DMRs |
| chr1 | 3506818 | 3507206 | 8.98E−08 | −40.35016571 | Hypo-methylated | NEPC\|CRPC-Adeno DMRs |
| chr1 | 3511792 | 3512244 | 4.25E−06 | −21.72374192 | Hypo-methylated | NEPC\|CRPC-Adeno DMRs |
| chr1 | 3607143 | 3607222 | 0.003525992 | −30.69110839 | Hypo-methylated | NEPC\|CRPC-Adeno DMRs |
| chr1 | 3615128 | 3615524 | 7.47E−05 | −26.03563074 | Hypo-methylated | NEPC\|CRPC-Adeno DMRs |
| chr1 | 3643797 | 3644749 | 4.65E−10 | 22.24736136 | Hyper-methylated | NEPC\|CRPC-Adeno DMRs |
| chr1 | 3669813 | 3672443 | 0.000400293 | −23.86206924 | Hypo-methylated | NEPC\|CRPC-Adeno DMRs |
| chr1 | 3679587 | 3684215 | 0.000221105 | −21.02997494 | Hypo-methylated | NEPC\|CRPC-Adeno DMRs |
| chr1 | 4773064 | 4774126 | 5.17E−06 | −24.5404572 | Hypo-methylated | NEPC\|CRPC-Adeno DMRs |
| chr1 | 5710356 | 5710691 | 0.001694183 | −29.8079148 | Hypo-methylated | NEPC\|CRPC-Adeno DMRs |
| chr1 | 5806929 | 5806981 | 0.001694183 | −26.20653021 | Hypo-methylated | NEPC\|CRPC-Adeno DMRs |
| chr1 | 6108507 | 6109003 | 0.009274146 | −21.65037594 | Hypo-methylated | NEPC\|CRPC-Adeno DMRs |
| chr1 | 6221905 | 6230389 | 0.000113382 | 21.93720158 | Hyper-methylated | NEPC\|CRPC-Adeno DMRs |
| chr1 | 6296631 | 6296737 | 0.000241282 | 22.67964912 | Hyper-methylated | NEPC\|CRPC-Adeno DMRs |
| chr1 | 6304286 | 6304652 | 0.002885308 | 22.36546053 | Hyper-methylated | NEPC\|CRPC-Adeno DMRs |
| chr1 | 6330487 | 6331864 | 5.17E−06 | −39.57825625 | Hypo-methylated | NEPC\|CRPC-Adeno DMRs |
| chr1 | 6438350 | 6438500 | 0.009458442 | −35.69498747 | Hypo-methylated | NEPC\|CRPC-Adeno DMRs |
| chr1 | 6445632 | 6445894 | 0.000176134 | −46.18077485 | Hypo-methylated | NEPC\|CRPC-Adeno DMRs |
| chr1 | 6533161 | 6534154 | 6.22E−12 | −40.84144832 | Hypo-methylated | NEPC\|CRPC-Adeno DMRs |
| chr1 | 6647655 | 6647751 | 0.000176134 | −22.19061195 | Hypo-methylated | NEPC\|CRPC-Adeno DMRs |
| chr1 | 6767641 | 6772318 | 0.003113626 | −40.2729439 | Hypo-methylated | NEPC\|CRPC-Adeno DMRs |
| chr1 | 6775316 | 6778626 | 0.009285355 | −22.56168746 | Hypo-methylated | NEPC\|CRPC-Adeno DMRs |
| chr1 | 6807362 | 6817716 | 0.001012154 | −28.38100644 | Hypo-methylated | NEPC\|CRPC-Adeno DMRs |
| chr1 | 7710979 | 7711108 | 0.000549237 | −36.08011696 | Hypo-methylated | NEPC\|CRPC-Adeno DMRs |
| chr1 | 7945526 | 7945651 | 0.001694183 | 23.27738791 | Hyper-methylated | NEPC\|CRPC-Adeno DMRs |
| chr1 | 8014086 | 8014624 | 1.10E−23 | 23.59429747 | Hyper-methylated | NEPC\|CRPC-Adeno DMRs |
| chr1 | 9130435 | 9131611 | 0.005737741 | 30.17114293 | Hyper-methylated | NEPC\|CRPC-Adeno DMRs |
| chr1 | 9376010 | 9376264 | 5.50E−05 | 22.04482619 | Hyper-methylated | NEPC\|CRPC-Adeno DMRs |
| chr1 | 9501650 | 9502882 | 0.000176134 | −45.19445704 | Hypo-methylated | NEPC\|CRPC-Adeno DMRs |
| chr1 | 9653880 | 9654098 | 0.00367696 | −23.84354067 | Hypo-methylated | NEPC\|CRPC-Adeno DMRs |
| chr1 | 9887816 | 9896089 | 0.000282282 | −26.58046784 | Hypo-methylated | NEPC\|CRPC-Adeno DMRs |
| chr1 | 10095202 | 10095654 | 0.001694183 | −34.18810916 | Hypo-methylated | NEPC\|CRPC-Adeno DMRs |
| chr1 | 10975754 | 10975909 | 0.001694183 | −33.5974537 | Hypo-methylated | NEPC\|CRPC-Adeno DMRs |
| chr1 | 11008316 | 11008961 | 5.36E−06 | −28.83703704 | Hypo-methylated | NEPC\|CRPC-Adeno DMRs |
| chr1 | 11435636 | 11436391 | 0.005737741 | −43.7377193 | Hypo-methylated | NEPC\|CRPC-Adeno DMRs |
| chr1 | 11696006 | 11708325 | 0.00222033 | 39.27577399 | Hyper-methylated | NEPC\|CRPC-Adeno DMRs |
| chr1 | 11713238 | 11713392 | 0.004750844 | −20.30618212 | Hypo-methylated | NEPC\|CRPC-Adeno DMRs |
| chr1 | 12113627 | 12123863 | 8.46E−07 | 27.11107314 | Hyper-methylated | NEPC\|CRPC-Adeno DMRs |
| chr1 | 13883902 | 13894019 | 0.001694183 | −51.90935673 | Hypo-methylated | NEPC\|CRPC-Adeno DMRs |
| chr1 | 13914270 | 13970164 | 0.006597219 | −35.58503899 | Hypo-methylated | NEPC\|CRPC-Adeno DMRs |
| chr1 | 14027548 | 14027604 | 0.001694183 | 36.17026942 | Hyper-methylated | NEPC\|CRPC-Adeno DMRs |
| chr1 | 15276468 | 15276617 | 0.000549237 | −28.97387288 | Hypo-methylated | NEPC\|CRPC-Adeno DMRs |
| chr1 | 15438936 | 15439152 | 0.002094266 | −20.35746774 | Hypo-methylated | NEPC\|CRPC-Adeno DMRs |
| chr1 | 15653145 | 15664647 | 0.000224358 | −31.87467382 | Hypo-methylated | NEPC\|CRPC-Adeno DMRs |
| chr1 | 15911071 | 15911703 | 3.12E−12 | −21.642335 | Hypo-methylated | NEPC\|CRPC-Adeno DMRs |
| chr1 | 16065023 | 16065205 | 0.00222033 | −22.90357143 | Hypo-methylated | NEPC\|CRPC-Adeno DMRs |
| chr1 | 16533719 | 16534683 | 0.001808548 | −27.31392544 | Hypo-methylated | NEPC\|CRPC-Adeno DMRs |
| chr1 | 17019941 | 17026803 | 1.37E−09 | 23.49319888 | Hyper-methylated | NEPC\|CRPC-Adeno DMRs |
| chr1 | 17186315 | 17186349 | 0.001694183 | 32.24912281 | Hyper-methylated | NEPC\|CRPC-Adeno DMRs |
| chr1 | 17306624 | 17307241 | 7.41E−07 | −21.71732572 | Hypo-methylated | NEPC\|CRPC-Adeno DMRs |
| chr1 | 17475708 | 17475923 | 1.94E−06 | 20.49690446 | Hyper-methylated | NEPC\|CRPC-Adeno DMRs |
| chr1 | 17488505 | 17489649 | 0.000484821 | −40.41052632 | Hypo-methylated | NEPC\|CRPC-Adeno DMRs |
| chr1 | 18032815 | 18033409 | 0.004750844 | −35.66290727 | Hypo-methylated | NEPC\|CRPC-Adeno DMRs |
| chr1 | 18606212 | 18606375 | 0.000184724 | −42.46725146 | Hypo-methylated | NEPC\|CRPC-Adeno DMRs |
| chr1 | 18808325 | 18812892 | 5.38E−38 | −53.2543711 | Hypo-methylated | NEPC\|CRPC-Adeno DMRs |
| chr1 | 19030039 | 19038813 | 0.008187764 | −25.93745817 | Hypo-methylated | NEPC\|CRPC-Adeno DMRs |
| chr1 | 19057778 | 19121141 | 9.16E−06 | −21.45587537 | Hypo-methylated | NEPC\|CRPC-Adeno DMRs |
| chr1 | 19186670 | 19194804 | 8.35E−06 | −31.62631579 | Hypo-methylated | NEPC\|CRPC-Adeno DMRs |
| chr1 | 19258538 | 19258624 | 0.001694183 | −35.89583333 | Hypo-methylated | NEPC\|CRPC-Adeno DMRs |
| chr1 | 19377393 | 19384828 | 2.04E−05 | −26.55734858 | Hypo-methylated | NEPC\|CRPC-Adeno DMRs |
| chr1 | 19664237 | 19664496 | 0.000253274 | −37.5188229 | Hypo-methylated | NEPC\|CRPC-Adeno DMRs |
| chr1 | 19676470 | 19686501 | 6.32E−08 | −30.33183761 | Hypo-methylated | NEPC\|CRPC-Adeno DMRs |
| chr1 | 19722252 | 19732801 | 2.91E−05 | −32.87718634 | Hypo-methylated | NEPC\|CRPC-Adeno DMRs |
| chr1 | 20140860 | 20142765 | 2.92E−14 | −47.89945554 | Hypo-methylated | NEPC\|CRPC-Adeno DMRs |
| chr1 | 20311214 | 20396751 | 0.002362173 | −20.34243181 | Hypo-methylated | NEPC\|CRPC-Adeno DMRs |
| chr1 | 20880205 | 20880241 | 0.000100851 | −24.98421053 | Hypo-methylated | NEPC\|CRPC-Adeno DMRs |
| chr1 | 21953521 | 21960152 | 0.002592833 | 26.54858942 | Hyper-methylated | NEPC\|CRPC-Adeno DMRs |
| chr1 | 22141031 | 22141342 | 5.77E−06 | 21.17186626 | Hyper-methylated | NEPC\|CRPC-Adeno DMRs |
| chr1 | 22194316 | 22195653 | 1.70E−05 | −41.16979323 | Hypo-methylated | NEPC\|CRPC-Adeno DMRs |
| chr1 | 22240923 | 22241839 | 4.34E−06 | −42.69576883 | Hypo-methylated | NEPC\|CRPC-Adeno DMRs |
| chr1 | 22260019 | 22260752 | 0.008187764 | −25.47755496 | Hypo-methylated | NEPC\|CRPC-Adeno DMRs |
| chr1 | 22511798 | 22511964 | 0.000926684 | −23.36574561 | Hypo-methylated | NEPC\|CRPC-Adeno DMRs |
| chr1 | 22854360 | 22854409 | 0.001532655 | −35.35723756 | Hypo-methylated | NEPC\|CRPC-Adeno DMRs |
| chr1 | 22920087 | 22920170 | 0.000176134 | −20.84671053 | Hypo-methylated | NEPC\|CRPC-Adeno DMRs |
| chr1 | 24463665 | 24469792 | 0.005899637 | −21.08105263 | Hypo-methylated | NEPC\|CRPC-Adeno DMRs |
| chr1 | 25231369 | 25233024 | 0.000324528 | −25.20504386 | Hypo-methylated | NEPC\|CRPC-Adeno DMRs |
| chr1 | 25522358 | 25525724 | 0.002885308 | −26.22349125 | Hypo-methylated | NEPC\|CRPC-Adeno DMRs |
| chr1 | 27845945 | 27846128 | 0.003327791 | −41.09010255 | Hypo-methylated | NEPC\|CRPC-Adeno DMRs |
| chr1 | 27961638 | 27961740 | 0.000340762 | 21.18438596 | Hyper-methylated | NEPC\|CRPC-Adeno DMRs |
| chr1 | 28871410 | 28871634 | 0.002976353 | −21.90300752 | Hypo-methylated | NEPC\|CRPC-Adeno DMRs |
| chr1 | 28918526 | 28919527 | 3.26E−13 | −22.35819416 | Hypo-methylated | NEPC\|CRPC-Adeno DMRs |
| chr1 | 29449832 | 29449957 | 0.000184724 | −21.13898635 | Hypo-methylated | NEPC\|CRPC-Adeno DMRs |
| chr1 | 30815151 | 30815694 | 0.000731112 | 26.74130781 | Hyper-methylated | NEPC\|CRPC-Adeno DMRs |
| chr1 | 32054406 | 32054842 | 3.30E−13 | −25.87491021 | Hypo-methylated | NEPC\|CRPC-Adeno DMRs |
| chr1 | 32180228 | 32180414 | 1.98E−05 | 28.07332533 | Hyper-methylated | NEPC\|CRPC-Adeno DMRs |
| chr1 | 32252165 | 32252427 | 5.50E−05 | −53.69590643 | Hypo-methylated | NEPC\|CRPC-Adeno DMRs |
| chr1 | 32714121 | 32714183 | 0.000100851 | −34.51866029 | Hypo-methylated | NEPC\|CRPC-Adeno DMRs |
| chr1 | 34082982 | 34083196 | 0.002094266 | −30.15178687 | Hypo-methylated | NEPC\|CRPC-Adeno DMRs |
| chr1 | 35297541 | 35311446 | 0.000586307 | −22.78102723 | Hypo-methylated | NEPC\|CRPC-Adeno DMRs |
| chr1 | 36549282 | 36549982 | 7.60E−14 | −27.36307949 | Hypo-methylated | NEPC\|CRPC-Adeno DMRs |
| chr1 | 37866213 | 37866383 | 0.000549237 | −39.09383754 | Hypo-methylated | NEPC\|CRPC-Adeno DMRs |
| chr1 | 38065716 | 38096144 | 0.000888 | −32.73975298 | Hypo-methylated | NEPC\|CRPC-Adeno DMRs |
| chr1 | 38113125 | 38133342 | 0.000397177 | −22.11663861 | Hypo-methylated | NEPC\|CRPC-Adeno DMRs |
| chr1 | 38230321 | 38230376 | 0.00041315 | −25.39824561 | Hypo-methylated | NEPC\|CRPC-Adeno DMRs |
| chr1 | 38461600 | 38461897 | 4.39E−13 | −35.85376096 | Hypo-methylated | NEPC\|CRPC-Adeno DMRs |
| chr1 | 38513525 | 38513670 | 2.46E−05 | 28.50680387 | Hyper-methylated | NEPC\|CRPC-Adeno DMRs |
| chr1 | 38955797 | 39012628 | 3.76E−05 | −21.94345422 | Hypo-methylated | NEPC\|CRPC-Adeno DMRs |
| chr1 | 39206321 | 39213090 | 0.004051703 | −22.63904423 | Hypo-methylated | NEPC\|CRPC-Adeno DMRs |
| chr1 | 40236936 | 40237158 | 2.01E−08 | 35.61952492 | Hyper-methylated | NEPC\|CRPC-Adeno DMRs |
| chr1 | 40421862 | 40426304 | 4.50E−05 | −23.77412281 | Hypo-methylated | NEPC\|CRPC-Adeno DMRs |
| chr1 | 41486102 | 41486256 | 5.99E−05 | −30.21579992 | Hypo-methylated | NEPC\|CRPC-Adeno DMRs |
| chr1 | 41857118 | 41857300 | 0.001694183 | −40.09153439 | Hypo-methylated | NEPC\|CRPC-Adeno DMRs |
| chr1 | 41967415 | 41967600 | 1.70E−05 | −25.39307018 | Hypo-methylated | NEPC\|CRPC-Adeno DMRs |
| chr1 | 44889376 | 44889443 | 0.000549237 | 29.21578947 | Hyper-methylated | NEPC\|CRPC-Adeno DMRs |
| chr1 | 45656666 | 45657053 | 5.01E−05 | −40.075 | Hypo-methylated | NEPC\|CRPC-Adeno DMRs |
| chr1 | 45768445 | 45768539 | 0.003113626 | −36.76111111 | Hypo-methylated | NEPC\|CRPC-Adeno DMRs |
| chr1 | 46956572 | 46959593 | 2.62E−07 | 23.52855116 | Hyper-methylated | NEPC\|CRPC-Adeno DMRs |
| chr1 | 47009603 | 47013241 | 2.16E−06 | 31.64829624 | Hyper-methylated | NEPC\|CRPC-Adeno DMRs |
| chr1 | 48237520 | 48241090 | 0.00017321 | −25.8764411 | Hypo-methylated | NEPC\|CRPC-Adeno DMRs |
| chr1 | 50741384 | 50741571 | 0.000176134 | −22.96381579 | Hypo-methylated | NEPC\|CRPC-Adeno DMRs |
| chr1 | 51763251 | 51763376 | 0.00222033 | −22.34802632 | Hypo-methylated | NEPC\|CRPC-Adeno DMRs |
| chr1 | 52832763 | 52832874 | 0.000549237 | −29.43289474 | Hypo-methylated | NEPC\|CRPC-Adeno DMRs |
| chr1 | 53785219 | 53785688 | 0.000549237 | −31.92932331 | Hypo-methylated | NEPC\|CRPC-Adeno DMRs |
| chr1 | 53841347 | 53842853 | 9.76E−12 | −50.50255221 | Hypo-methylated | NEPC\|CRPC-Adeno DMRs |
| chr1 | 54072597 | 54072638 | 0.001694183 | −36.6622807 | Hypo-methylated | NEPC\|CRPC-Adeno DMRs |
| chr1 | 54739154 | 54739199 | 0.000549237 | −21.88703008 | Hypo-methylated | NEPC\|CRPC-Adeno DMRs |
| chr1 | 54808693 | 54808777 | 5.50E−05 | −46.87089019 | Hypo-methylated | NEPC\|CRPC-Adeno DMRs |
| chr1 | 55075333 | 55089183 | 0.001310325 | −24.9670668 | Hypo-methylated | NEPC\|CRPC-Adeno DMRs |
| chr1 | 55461536 | 55462990 | 0.001310325 | 25.88825911 | Hyper-methylated | NEPC\|CRPC-Adeno DMRs |
| chr1 | 55525673 | 55530145 | 2.34E−07 | −28.42802802 | Hypo-methylated | NEPC\|CRPC-Adeno DMRs |
| chr1 | 65431241 | 65431341 | 0.00041315 | −23.72631579 | Hypo-methylated | NEPC\|CRPC-Adeno DMRs |
| chr1 | 65731412 | 65731782 | 7.63E−11 | 33.9382758 | Hyper-methylated | NEPC\|CRPC-Adeno DMRs |
| chr1 | 67773363 | 67773701 | 2.60E−17 | −20.3537604 | Hypo-methylated | NEPC\|CRPC-Adeno DMRs |
| chr1 | 68219766 | 68219961 | 0.004750844 | −24.62124499 | Hypo-methylated | NEPC\|CRPC-Adeno DMRs |
| chr1 | 68281422 | 68281687 | 0.001694183 | 30.75108359 | Hyper-methylated | NEPC\|CRPC-Adeno DMRs |
| chr1 | 70598406 | 70598450 | 0.005737741 | 24.49444444 | Hyper-methylated | NEPC\|CRPC-Adeno DMRs |
| chr1 | 79472483 | 79472537 | 0.001694183 | 20.78752437 | Hyper-methylated | NEPC\|CRPC-Adeno DMRs |
| chr1 | 81564805 | 81565000 | 0.006785531 | −20.41785453 | Hypo-methylated | NEPC\|CRPC-Adeno DMRs |
| chr1 | 81869776 | 81869957 | 0.001694183 | 41.56666667 | Hyper-methylated | NEPC\|CRPC-Adeno DMRs |
| chr1 | 85527430 | 85527873 | 0.003113626 | −21.43333333 | Hypo-methylated | NEPC\|CRPC-Adeno DMRs |
| chr1 | 88928458 | 88928533 | 4.94E−05 | 28.47192982 | Hyper-methylated | NEPC\|CRPC-Adeno DMRs |
| chr1 | 88932816 | 88933104 | 1.92E−07 | −36.25618461 | Hypo-methylated | NEPC\|CRPC-Adeno DMRs |
| chr1 | 89016942 | 89103353 | 0.002376793 | −26.64570785 | Hypo-methylated | NEPC\|CRPC-Adeno DMRs |
| chr1 | 90075469 | 90076027 | 0.003113626 | −24.3662424 | Hypo-methylated | NEPC\|CRPC-Adeno DMRs |
| chr1 | 90099695 | 90099891 | 0.001694183 | −20.95272904 | Hypo-methylated | NEPC\|CRPC-Adeno DMRs |
| chr1 | 91182462 | 91185367 | 1.56E−24 | 28.6925175 | Hyper-methylated | NEPC\|CRPC-Adeno DMRs |
| chr1 | 91190946 | 91190985 | 0.003525992 | 29.62017544 | Hyper-methylated | NEPC\|CRPC-Adeno DMRs |
| chr1 | 94566449 | 94568090 | 0.000542154 | 24.67751196 | Hyper-methylated | NEPC\|CRPC-Adeno DMRs |
| chr1 | 98510937 | 98511106 | 4.76E−10 | 34.227618 | Hyper-methylated | NEPC\|CRPC-Adeno DMRs |
| chr1 | 99470311 | 99470639 | 3.73E−11 | 27.29306861 | Hyper-methylated | NEPC\|CRPC-Adeno DMRs |
| chr1 | 103215035 | 103268330 | 5.50E−05 | −30.66237817 | Hypo-methylated | NEPC\|CRPC-Adeno DMRs |
| chr1 | 106675550 | 106926243 | 0.00222033 | −20.93739427 | Hypo-methylated | NEPC\|CRPC-Adeno DMRs |
| chr1 | 106982881 | 107050285 | 1.60E−06 | −26.43186671 | Hypo-methylated | NEPC\|CRPC-Adeno DMRs |
| chr1 | 107536775 | 107556249 | 0.002976353 | −27.24135338 | Hypo-methylated | NEPC\|CRPC-Adeno DMRs |
| chr1 | 110883147 | 110884779 | 0.000128281 | −21.83186449 | Hypo-methylated | NEPC\|CRPC-Adeno DMRs |
| chr1 | 111218002 | 111218083 | 0.001802623 | 20.78788638 | Hyper-methylated | NEPC\|CRPC-Adeno DMRs |
| chr1 | 112282928 | 112283151 | 2.16E−12 | −21.33957115 | Hypo-methylated | NEPC\|CRPC-Adeno DMRs |
| chr1 | 114654380 | 114654573 | 0.000549237 | −28.91853654 | Hypo-methylated | NEPC\|CRPC-Adeno DMRs |
| chr1 | 116197674 | 116197814 | 0.000549237 | 40.82951128 | Hyper-methylated | NEPC\|CRPC-Adeno DMRs |
| chr1 | 144994610 | 144994895 | 0.000348917 | −24.56340852 | Hypo-methylated | NEPC\|CRPC-Adeno DMRs |
| chr1 | 145514968 | 145515165 | 0.000200845 | −32.19906433 | Hypo-methylated | NEPC\|CRPC-Adeno DMRs |
| chr1 | 149333101 | 149333199 | 0.0001635 | 20.4851143 | Hyper-methylated | NEPC\|CRPC-Adeno DMRs |
| chr1 | 150254716 | 150255542 | 8.64E−11 | −20.22602339 | Hypo-methylated | NEPC\|CRPC-Adeno DMRs |
| chr1 | 151105124 | 151105181 | 0.005737741 | 20.08508772 | Hyper-methylated | NEPC\|CRPC-Adeno DMRs |
| chr1 | 151369847 | 151371481 | 0.007379618 | −24.81473684 | Hypo-methylated | NEPC\|CRPC-Adeno DMRs |
| chr1 | 153505362 | 153539059 | 1.50E−07 | −23.35227554 | Hypo-methylated | NEPC\|CRPC-Adeno DMRs |
| chr1 | 153748967 | 153749213 | 0.005239183 | −39.21106934 | Hypo-methylated | NEPC\|CRPC-Adeno DMRs |
| chr1 | 153768106 | 153768185 | 4.74E−05 | −22.12099947 | Hypo-methylated | NEPC\|CRPC-Adeno DMRs |
| chr1 | 154259925 | 154273412 | 5.50E−05 | −26.2675141 | Hypo-methylated | NEPC\|CRPC-Adeno DMRs |
| chr1 | 154386862 | 154390505 | 0.00217795 | 26.50596491 | Hyper-methylated | NEPC\|CRPC-Adeno DMRs |
| chr1 | 154474108 | 154474751 | 2.73E−35 | −21.6292707 | Hypo-methylated | NEPC\|CRPC-Adeno DMRs |
| chr1 | 154840396 | 154841951 | 8.52E−07 | −37.19461812 | Hypo-methylated | NEPC\|CRPC-Adeno DMRs |
| chr1 | 154978298 | 154986712 | 0.003327791 | 25.91245079 | Hyper-methylated | NEPC\|CRPC-Adeno DMRs |
| chr1 | 155103222 | 155104003 | 0.001694183 | 48.74731682 | Hyper-methylated | NEPC\|CRPC-Adeno DMRs |
| chr1 | 155220184 | 155220232 | 5.17E−06 | 23.78229665 | Hyper-methylated | NEPC\|CRPC-Adeno DMRs |
| chr1 | 155243891 | 155243940 | 0.001694183 | −33.39105392 | Hypo-methylated | NEPC\|CRPC-Adeno DMRs |
| chr1 | 155262968 | 155263101 | 0.005737741 | −27.63859649 | Hypo-methylated | NEPC\|CRPC-Adeno DMRs |
| chr1 | 155912449 | 155915866 | 0.000890838 | −31.15620055 | Hypo-methylated | NEPC\|CRPC-Adeno DMRs |
| chr1 | 156146474 | 156146805 | 0.00041315 | −22.11580476 | Hypo-methylated | NEPC\|CRPC-Adeno DMRs |
| chr1 | 156379461 | 156390252 | 0.000581135 | 25.14437135 | Hyper-methylated | NEPC\|CRPC-Adeno DMRs |
| chr1 | 156390706 | 156390914 | 1.11E−06 | −21.13623239 | Hypo-methylated | NEPC\|CRPC-Adeno DMRs |
| chr1 | 156593608 | 156593887 | 1.49E−07 | −26.00902446 | Hypo-methylated | NEPC\|CRPC-Adeno DMRs |
| chr1 | 156646419 | 156646946 | 5.72E−09 | −20.03551302 | Hypo-methylated | NEPC\|CRPC-Adeno DMRs |
| chr1 | 156890278 | 156890488 | 1.45E−06 | −22.45786894 | Hypo-methylated | NEPC\|CRPC-Adeno DMRs |
| chr1 | 158150797 | 158151205 | 8.34E−06 | 21.72869609 | Hyper-methylated | NEPC\|CRPC-Adeno DMRs |
| chr1 | 160094101 | 160104377 | 0.001125639 | −27.00631579 | Hypo-methylated | NEPC\|CRPC-Adeno DMRs |
| chr1 | 161448889 | 161471766 | 0.001802623 | −42.54278343 | Hypo-methylated | NEPC\|CRPC-Adeno DMRs |
| chr1 | 161592118 | 161674114 | 0.000484821 | −25.12484532 | Hypo-methylated | NEPC\|CRPC-Adeno DMRs |
| chr1 | 167408692 | 167409105 | 1.90E−12 | −20.35913351 | Hypo-methylated | NEPC\|CRPC-Adeno DMRs |
| chr1 | 167458187 | 167471283 | 0.000209051 | −29.41777455 | Hypo-methylated | NEPC\|CRPC-Adeno DMRs |
| chr1 | 167881539 | 167898361 | 0.001012154 | 32.31248956 | Hyper-methylated | NEPC\|CRPC-Adeno DMRs |
| chr1 | 168054725 | 168054875 | 0.000549237 | 34.55829771 | Hyper-methylated | NEPC\|CRPC-Adeno DMRs |
| chr1 | 169707660 | 169709862 | 0.003327791 | −20.90998882 | Hypo-methylated | NEPC\|CRPC-Adeno DMRs |
| chr1 | 170883644 | 171350978 | 5.08E−08 | −30.69585507 | Hypo-methylated | NEPC\|CRPC-Adeno DMRs |
| chr1 | 171625172 | 171640038 | 0.0001415 | −20.49778488 | Hypo-methylated | NEPC\|CRPC-Adeno DMRs |
| chr1 | 173491184 | 173491286 | 0.000549237 | −21.71888545 | Hypo-methylated | NEPC\|CRPC-Adeno DMRs |
| chr1 | 177056919 | 177113360 | 0.001802623 | 27.68604845 | Hyper-methylated | NEPC\|CRPC-Adeno DMRs |
| chr1 | 177183426 | 177219623 | 1.29E−09 | 22.82347189 | Hyper-methylated | NEPC\|CRPC-Adeno DMRs |
| chr1 | 177247737 | 177716521 | 3.00E−08 | 21.83378655 | Hyper-methylated | NEPC\|CRPC-Adeno DMRs |
| chr1 | 179388196 | 179497888 | 3.58E−07 | −32.18985507 | Hypo-methylated | NEPC\|CRPC-Adeno DMRs |
| chr1 | 179713513 | 179740376 | 3.74E−05 | 25.3960944 | Hyper-methylated | NEPC\|CRPC-Adeno DMRs |
| chr1 | 179742849 | 179743483 | 0.001694183 | −23.67641326 | Hypo-methylated | NEPC\|CRPC-Adeno DMRs |
| chr1 | 180204579 | 180204974 | 2.19E−08 | 21.53567406 | Hyper-methylated | NEPC\|CRPC-Adeno DMRs |
| chr1 | 181452972 | 181453056 | 1.55E−06 | 26.27807018 | Hyper-methylated | NEPC\|CRPC-Adeno DMRs |
| chr1 | 183155057 | 183169382 | 2.08E−08 | −24.03792361 | Hypo-methylated | NEPC\|CRPC-Adeno DMRs |
| chr1 | 186889118 | 186949281 | 0.001642117 | −23.16023392 | Hypo-methylated | NEPC\|CRPC-Adeno DMRs |
| chr1 | 188810539 | 188932110 | 0.00158762 | 21.05204678 | Hyper-methylated | NEPC\|CRPC-Adeno DMRs |
| chr1 | 197879662 | 197879802 | 1.55E−06 | 31.09429825 | Hyper-methylated | NEPC\|CRPC-Adeno DMRs |
| chr1 | 197887955 | 197890813 | 1.21E−06 | 23.51822284 | Hyper-methylated | NEPC\|CRPC-Adeno DMRs |
| chr1 | 200992306 | 200992549 | 1.55E−08 | −22.36142788 | Hypo-methylated | NEPC\|CRPC-Adeno DMRs |
| chr1 | 201096142 | 201096334 | 4.74E−05 | −37.35956558 | Hypo-methylated | NEPC\|CRPC-Adeno DMRs |
| chr1 | 204827934 | 204839816 | 0.00060921 | −20.76413829 | Hypo-methylated | NEPC\|CRPC-Adeno DMRs |
| chr1 | 205011154 | 205012570 | 9.27E−10 | −27.80571518 | Hypo-methylated | NEPC\|CRPC-Adeno DMRs |
| chr1 | 205424899 | 205424973 | 0.000318709 | 20.82020202 | Hyper-methylated | NEPC\|CRPC-Adeno DMRs |
| chr1 | 206837257 | 206837409 | 0.000953892 | −27.04917763 | Hypo-methylated | NEPC\|CRPC-Adeno DMRs |
| chr1 | 207842658 | 207843013 | 1.84E−08 | 32.73795723 | Hyper-methylated | NEPC\|CRPC-Adeno DMRs |
| chr1 | 208202787 | 208202866 | 0.001694183 | −37.34385965 | Hypo-methylated | NEPC\|CRPC-Adeno DMRs |
| chr1 | 208255452 | 208255712 | 1.70E−05 | −28.36458333 | Hypo-methylated | NEPC\|CRPC-Adeno DMRs |
| chr1 | 209548645 | 209785461 | 3.55E−06 | −28.7670178 | Hypo-methylated | NEPC\|CRPC-Adeno DMRs |
| chr1 | 209803066 | 209803175 | 0.000176134 | −32.63460505 | Hypo-methylated | NEPC\|CRPC-Adeno DMRs |
| chr1 | 209920990 | 209921100 | 0.003113626 | 30.05701754 | Hyper-methylated | NEPC\|CRPC-Adeno DMRs |
| chr1 | 210111212 | 210111944 | 2.63E−25 | 32.60420009 | Hyper-methylated | NEPC\|CRPC-Adeno DMRs |
| chr1 | 210426095 | 210460305 | 5.75E−05 | −21.41190415 | Hypo-methylated | NEPC\|CRPC-Adeno DMRs |
| chr1 | 210526652 | 210703855 | 0.005375697 | −22.98817755 | Hypo-methylated | NEPC\|CRPC-Adeno DMRs |
| chr1 | 210806711 | 210825862 | 0.002976353 | −37.24840252 | Hypo-methylated | NEPC\|CRPC-Adeno DMRs |
| chr1 | 213124496 | 213124946 | 1.06E−08 | −21.28849903 | Hypo-methylated | NEPC\|CRPC-Adeno DMRs |
| chr1 | 215256112 | 215256635 | 8.27E−26 | 25.93420627 | Hyper-methylated | NEPC\|CRPC-Adeno DMRs |
| chr1 | 218098336 | 218098399 | 1.09E−07 | 27.77977839 | Hyper-methylated | NEPC\|CRPC-Adeno DMRs |
| chr1 | 220636185 | 220636483 | 0.000101637 | −35.45906433 | Hypo-methylated | NEPC\|CRPC-Adeno DMRs |
| chr1 | 220826507 | 220826565 | 0.001694183 | −22.90150376 | Hypo-methylated | NEPC\|CRPC-Adeno DMRs |
| chr1 | 224372513 | 224382834 | 0.00678631 | 25.86671335 | Hyper-methylated | NEPC\|CRPC-Adeno DMRs |
| chr1 | 225629948 | 225631281 | 0.001012154 | −28.46495177 | Hypo-methylated | NEPC\|CRPC-Adeno DMRs |
| chr1 | 226003504 | 226003849 | 0.000303685 | −27.39051332 | Hypo-methylated | NEPC\|CRPC-Adeno DMRs |
| chr1 | 226075069 | 226075767 | 0.002667178 | 21.49674536 | Hyper-methylated | NEPC\|CRPC-Adeno DMRs |
| chr1 | 226090654 | 226099251 | 0.000154511 | 26.70270853 | Hyper-methylated | NEPC\|CRPC-Adeno DMRs |
| chr1 | 226126979 | 226127446 | 2.23E−05 | 22.66344507 | Hyper-methylated | NEPC\|CRPC-Adeno DMRs |
| chr1 | 228261545 | 228263625 | 0.002376793 | −33.74621838 | Hypo-methylated | NEPC\|CRPC-Adeno DMRs |
| chr1 | 228463266 | 228463571 | 3.68E−07 | −24.47091413 | Hypo-methylated | NEPC\|CRPC-Adeno DMRs |
| chr1 | 228492413 | 228502871 | 6.49E−06 | −21.71374269 | Hypo-methylated | NEPC\|CRPC-Adeno DMRs |
| chr1 | 228504625 | 228506357 | 0.000763207 | −20.34883041 | Hypo-methylated | NEPC\|CRPC-Adeno DMRs |
| chr1 | 229071337 | 229071545 | 0.001694183 | −20.70779727 | Hypo-methylated | NEPC\|CRPC-Adeno DMRs |
| chr1 | 229506415 | 229517095 | 0.009274146 | −27.25232456 | Hypo-methylated | NEPC\|CRPC-Adeno DMRs |
| chr1 | 229567994 | 229569644 | 9.72E−17 | 26.89497624 | Hyper-methylated | NEPC\|CRPC-Adeno DMRs |
| chr1 | 229570004 | 229570597 | 1.09E−08 | −25.87887427 | Hypo-methylated | NEPC\|CRPC-Adeno DMRs |
| chr1 | 230266192 | 230266405 | 0.000549237 | −47.26333691 | Hypo-methylated | NEPC\|CRPC-Adeno DMRs |
| chr1 | 230436736 | 230436927 | 0.000200845 | −24.14106725 | Hypo-methylated | NEPC\|CRPC-Adeno DMRs |
| chr1 | 230849738 | 230850212 | 0.003087144 | 20.44572471 | Hyper-methylated | NEPC\|CRPC-Adeno DMRs |
| chr1 | 231299496 | 231299678 | 3.71E−11 | 25.4944332 | Hyper-methylated | NEPC\|CRPC-Adeno DMRs |
| chr1 | 231826172 | 231829667 | 3.72E−06 | −29.1713835 | Hypo-methylated | NEPC\|CRPC-Adeno DMRs |
| chr1 | 232463565 | 232463621 | 0.000832692 | −27.09013158 | Hypo-methylated | NEPC\|CRPC-Adeno DMRs |
| chr1 | 234831584 | 234845383 | 0.002094266 | −45.37348928 | Hypo-methylated | NEPC\|CRPC-Adeno DMRs |
| chr1 | 234908226 | 234908382 | 0.000549237 | −22.76853741 | Hypo-methylated | NEPC\|CRPC-Adeno DMRs |
| chr1 | 236306856 | 236308302 | 0.004693517 | 22.85116846 | Hyper-methylated | NEPC\|CRPC-Adeno DMRs |
| chr1 | 236849487 | 236850241 | 7.80E−25 | 27.84412585 | Hyper-methylated | NEPC\|CRPC-Adeno DMRs |
| chr1 | 236857562 | 236908311 | 1.30E−07 | −36.01468551 | Hypo-methylated | NEPC\|CRPC-Adeno DMRs |
| chr1 | 237034318 | 237052704 | 0.001694183 | 22.39610136 | Hyper-methylated | NEPC\|CRPC-Adeno DMRs |
| chr1 | 237122944 | 237195447 | 7.25E−10 | −42.84443736 | Hypo-methylated | NEPC\|CRPC-Adeno DMRs |
| chr1 | 237205222 | 237206562 | 4.39E−32 | 25.56940787 | Hyper-methylated | NEPC\|CRPC-Adeno DMRs |
| chr1 | 237212338 | 237832931 | 7.55E−31 | −40.21227117 | Hypo-methylated | NEPC\|CRPC-Adeno DMRs |
| chr1 | 237832994 | 237959047 | 0.001802623 | −21.93031537 | Hypo-methylated | NEPC\|CRPC-Adeno DMRs |
| chr1 | 239560828 | 239931256 | 7.16E−11 | −28.97652329 | Hypo-methylated | NEPC\|CRPC-Adeno DMRs |
| chr1 | 242633393 | 242674072 | 0.002718013 | −20.45013645 | Hypo-methylated | NEPC\|CRPC-Adeno DMRs |
| chr1 | 243646523 | 243647205 | 0.000876403 | 25.17284516 | Hyper-methylated | NEPC\|CRPC-Adeno DMRs |
| chr1 | 244106446 | 244189663 | 0.002094266 | −22.74063629 | Hypo-methylated | NEPC\|CRPC-Adeno DMRs |
| chr1 | 244394659 | 244395366 | 0.001694183 | −33.49080954 | Hypo-methylated | NEPC\|CRPC-Adeno DMRs |
| chr1 | 245375798 | 245376090 | 0.00041315 | −28.24485055 | Hypo-methylated | NEPC\|CRPC-Adeno DMRs |
| chr1 | 245397978 | 245398214 | 5.36E−06 | −43.2464425 | Hypo-methylated | NEPC\|CRPC-Adeno DMRs |
| chr1 | 246311010 | 246311301 | 0.000581135 | −27.66183297 | Hypo-methylated | NEPC\|CRPC-Adeno DMRs |
| chr1 | 246958344 | 246958526 | 0.000324528 | −35.9125 | Hypo-methylated | NEPC\|CRPC-Adeno DMRs |
| chr1 | 247654811 | 247694461 | 4.08E−22 | 22.6762879 | Hyper-methylated | NEPC\|CRPC-Adeno DMRs |
| chr2 | 558070 | 565808 | 0.005899637 | −20.64210483 | Hypo-methylated | NEPC\|CRPC-Adeno DMRs |
| chr2 | 574111 | 575374 | 0.004750844 | −31.08940058 | Hypo-methylated | NEPC\|CRPC-Adeno DMRs |
| chr2 | 587269 | 594893 | 0.000153872 | −20.88201546 | Hypo-methylated | NEPC\|CRPC-Adeno DMRs |
| chr2 | 946912 | 947141 | 0.003223004 | 20.12491676 | Hyper-methylated | NEPC\|CRPC-Adeno DMRs |
| chr2 | 1229230 | 1229292 | 0.001694183 | −22.63308271 | Hypo-methylated | NEPC\|CRPC-Adeno DMRs |
| chr2 | 3704747 | 3704865 | 0.000176134 | −33.80723684 | Hypo-methylated | NEPC\|CRPC-Adeno DMRs |
| chr2 | 5833641 | 5834110 | 6.46E−08 | 22.55885167 | Hyper-methylated | NEPC\|CRPC-Adeno DMRs |
| chr2 | 7497762 | 7518683 | 0.006785531 | −38.46185516 | Hypo-methylated | NEPC\|CRPC-Adeno DMRs |
| chr2 | 8793681 | 8797420 | 0.001802623 | −26.99752954 | Hypo-methylated | NEPC\|CRPC-Adeno DMRs |
| chr2 | 9324021 | 9327608 | 4.29E−06 | 21.55963542 | Hyper-methylated | NEPC\|CRPC-Adeno DMRs |
| chr2 | 9513262 | 9513322 | 0.001694183 | −30.67050961 | Hypo-methylated | NEPC\|CRPC-Adeno DMRs |
| chr2 | 10170502 | 10170669 | 0.000176134 | −33.37368421 | Hypo-methylated | NEPC\|CRPC-Adeno DMRs |
| chr2 | 10302076 | 10303145 | 0.000324528 | −35.79671053 | Hypo-methylated | NEPC\|CRPC-Adeno DMRs |
| chr2 | 10899354 | 10899571 | 0.002094266 | 28.30116959 | Hyper-methylated | NEPC\|CRPC-Adeno DMRs |
| chr2 | 11925058 | 11925198 | 0.00324027 | 24.70484893 | Hyper-methylated | NEPC\|CRPC-Adeno DMRs |
| chr2 | 15936602 | 15940517 | 0.004750844 | −21.02404971 | Hypo-methylated | NEPC\|CRPC-Adeno DMRs |
| chr2 | 16153331 | 16154366 | 1.69E−10 | −23.34380604 | Hypo-methylated | NEPC\|CRPC-Adeno DMRs |
| chr2 | 16511319 | 16578454 | 0.000101574 | −20.5190639 | Hypo-methylated | NEPC\|CRPC-Adeno DMRs |
| chr2 | 17421705 | 17524336 | 0.004750844 | −29.73421053 | Hypo-methylated | NEPC\|CRPC-Adeno DMRs |
| chr2 | 20548540 | 20548615 | 0.000176134 | −20.26578947 | Hypo-methylated | NEPC\|CRPC-Adeno DMRs |
| chr2 | 21056358 | 21101016 | 0.006847068 | −34.48125522 | Hypo-methylated | NEPC\|CRPC-Adeno DMRs |
| chr2 | 21266324 | 21267030 | 8.36E−05 | 23.78680574 | Hyper-methylated | NEPC\|CRPC-Adeno DMRs |
| chr2 | 23167191 | 23167321 | 0.005737741 | −21.97819201 | Hypo-methylated | NEPC\|CRPC-Adeno DMRs |
| chr2 | 23747138 | 23757045 | 5.04E−05 | −30.68748173 | Hypo-methylated | NEPC\|CRPC-Adeno DMRs |
| chr2 | 23770125 | 23777570 | 1.50E−07 | −34.75308642 | Hypo-methylated | NEPC\|CRPC-Adeno DMRs |
| chr2 | 23786468 | 23788930 | 0.002927544 | −21.08622482 | Hypo-methylated | NEPC\|CRPC-Adeno DMRs |
| chr2 | 25285553 | 25299930 | 0.000667554 | −42.10759288 | Hypo-methylated | NEPC\|CRPC-Adeno DMRs |
| chr2 | 25452256 | 25459585 | 7.36E−07 | −30.8897254 | Hypo-methylated | NEPC\|CRPC-Adeno DMRs |
| chr2 | 26407468 | 26408214 | 1.64E−30 | −25.46531281 | Hypo-methylated | NEPC\|CRPC-Adeno DMRs |
| chr2 | 26681020 | 26681087 | 0.001694183 | −25.86666667 | Hypo-methylated | NEPC\|CRPC-Adeno DMRs |
| chr2 | 26763381 | 26766237 | 0.000324528 | −25.56650219 | Hypo-methylated | NEPC\|CRPC-Adeno DMRs |
| chr2 | 27165508 | 27165544 | 0.000721524 | 21.47744361 | Hyper-methylated | NEPC\|CRPC-Adeno DMRs |
| chr2 | 27473163 | 27473238 | 5.36E−06 | 33.97272348 | Hyper-methylated | NEPC\|CRPC-Adeno DMRs |
| chr2 | 28907245 | 28907308 | 0.000549237 | −31.74135338 | Hypo-methylated | NEPC\|CRPC-Adeno DMRs |
| chr2 | 29192790 | 29213525 | 2.76E−05 | −22.80890688 | Hypo-methylated | NEPC\|CRPC-Adeno DMRs |
| chr2 | 29232329 | 29273929 | 3.07E−08 | −23.33551665 | Hypo-methylated | NEPC\|CRPC-Adeno DMRs |
| chr2 | 29285106 | 29320167 | 2.82E−06 | −21.43834858 | Hypo-methylated | NEPC\|CRPC-Adeno DMRs |
| chr2 | 29438307 | 29448419 | 0.002576454 | −25.66365272 | Hypo-methylated | NEPC\|CRPC-Adeno DMRs |
| chr2 | 29563311 | 29722569 | 0.00060921 | −27.92785804 | Hypo-methylated | NEPC\|CRPC-Adeno DMRs |
| chr2 | 30158882 | 30328159 | 1.99E−06 | −23.881619 | Hypo-methylated | NEPC\|CRPC-Adeno DMRs |
| chr2 | 31231690 | 31231895 | 0.001549499 | −25.8625 | Hypo-methylated | NEPC\|CRPC-Adeno DMRs |
| chr2 | 32483098 | 32493184 | 1.46E−05 | −24.73126198 | Hypo-methylated | NEPC\|CRPC-Adeno DMRs |
| chr2 | 33835961 | 33979130 | 2.26E−06 | −21.8558705 | Hypo-methylated | NEPC\|CRPC-Adeno DMRs |
| chr2 | 34367845 | 34373093 | 0.000549237 | −26.02518551 | Hypo-methylated | NEPC\|CRPC-Adeno DMRs |
| chr2 | 34615317 | 34615366 | 0.004750844 | −21.01269841 | Hypo-methylated | NEPC\|CRPC-Adeno DMRs |
| chr2 | 37571704 | 37572366 | 2.01E−18 | −24.71985052 | Hypo-methylated | NEPC\|CRPC-Adeno DMRs |
| chr2 | 39471098 | 39471148 | 0.00041315 | −29.06169973 | Hypo-methylated | NEPC\|CRPC-Adeno DMRs |
| chr2 | 43038039 | 43038270 | 8.97E−08 | −20.15104428 | Hypo-methylated | NEPC\|CRPC-Adeno DMRs |
| chr2 | 43327732 | 43328756 | 6.98E−17 | −27.55103368 | Hypo-methylated | NEPC\|CRPC-Adeno DMRs |
| chr1 | 44058915 | 44059267 | 6.05E−10 | −23.58462578 | Hypo-methylated | NEPC\|CRPC-Adeno DMRs |
| chr1 | 47877314 | 47904072 | 0.003348504 | −22.79426344 | Hypo-methylated | NEPC\|CRPC-Adeno DMRs |
| chr1 | 54010084 | 54010295 | 0.001694183 | 26.45233918 | Hyper-methylated | NEPC\|CRPC-Adeno DMRs |
| chr2 | 54685003 | 54685052 | 0.001694183 | −24.97791005 | Hypo-methylated | NEPC\|CRPC-Adeno DMRs |
| chr2 | 58654887 | 58655598 | 4.11E−07 | 21.53544974 | Hyper-methylated | NEPC\|CRPC-Adeno DMRs |
| chr2 | 62221214 | 62221247 | 0.005737741 | 20.79824561 | Hyper-methylated | NEPC\|CRPC-Adeno DMRs |
| chr2 | 65217164 | 65217742 | 0.001666853 | −32.39952153 | Hypo-methylated | NEPC\|CRPC-Adeno DMRs |
| chr2 | 66804656 | 66810625 | 3.38E−08 | 31.89104224 | Hyper-methylated | NEPC\|CRPC-Adeno DMRs |
| chr2 | 73301681 | 73308018 | 1.98E−05 | 23.24652979 | Hyper-methylated | NEPC\|CRPC-Adeno DMRs |
| chr1 | 74006134 | 74006203 | 0.000549237 | −23.42011278 | Hypo-methylated | NEPC\|CRPC-Adeno DMRs |
| chr1 | 77961147 | 77962203 | 5.50E−05 | 20.36482231 | Hyper-methylated | NEPC\|CRPC-Adeno DMRs |
| chr1 | 79918776 | 79972327 | 0.000549237 | −26.724914 | Hypo-methylated | NEPC\|CRPC-Adeno DMRs |
| chr1 | 80519910 | 80530588 | 6.32E−08 | 28.74203873 | Hyper-methylated | NEPC\|CRPC-Adeno DMRs |
| chr1 | 80531759 | 80531809 | 1.55E−06 | 20.3504386 | Hyper-methylated | NEPC\|CRPC-Adeno DMRs |
| chr1 | 81424285 | 81694490 | 4.01E−05 | −24.8101957 | Hypo-methylated | NEPC\|CRPC-Adeno DMRs |
| chr1 | 81694557 | 81850017 | 7.45E−10 | −28.2601711 | Hypo-methylated | NEPC\|CRPC-Adeno DMRs |
| chr1 | 84770004 | 85043184 | 3.10E−06 | −24.46461988 | Hypo-methylated | NEPC\|CRPC-Adeno DMRs |
| chr1 | 87015911 | 87017295 | 1.53E−15 | 24.99944686 | Hyper-methylated | NEPC\|CRPC-Adeno DMRs |
| chr1 | 88305001 | 88305825 | 0.003525992 | −20.39278752 | Hypo-methylated | NEPC\|CRPC-Adeno DMRs |
| chr1 | 88583923 | 88583990 | 0.001545937 | 23.94371345 | Hyper-methylated | NEPC\|CRPC-Adeno DMRs |
| chr1 | 88650149 | 88650678 | 0.002309241 | 25.4040499 | Hyper-methylated | NEPC\|CRPC-Adeno DMRs |
| chr1 | 89064665 | 89064762 | 9.82E−05 | 21.048583 | Hyper-methylated | NEPC\|CRPC-Adeno DMRs |
| chr1 | 91757861 | 91757982 | 8.87E−06 | 27.79373782 | Hyper-methylated | NEPC\|CRPC-Adeno DMRs |
| chr1 | 93018365 | 92028390 | 0.001012154 | 27.52294339 | Hyper-methylated | NEPC\|CRPC-Adeno DMRs |
| chr1 | 95541756 | 95584028 | 0.005532731 | −23.90266734 | Hypo-methylated | NEPC\|CRPC-Adeno DMRs |
| chr1 | 95935759 | 95942618 | 0.001306789 | 21.97767067 | Hyper-methylated | NEPC\|CRPC-Adeno DMRs |
| chr1 | 96053259 | 96055375 | 9.74E−06 | −25.70678298 | Hypo-methylated | NEPC\|CRPC-Adeno DMRs |
| chr1 | 96357350 | 96421972 | 0.007136365 | −26.93834586 | Hypo-methylated | NEPC\|CRPC-Adeno DMRs |
| chr1 | 99531119 | 99531917 | 5.36E−06 | −38.45979532 | Hypo-methylated | NEPC\|CRPC-Adeno DMRs |
| chr1 | 100863650 | 100864133 | 0.001642117 | −20.34782326 | Hypo-methylated | NEPC\|CRPC-Adeno DMRs |
| chr1 | 101726347 | 101746674 | 0.00450545 | −27.78424493 | Hypo-methylated | NEPC\|CRPC-Adeno DMRs |
| chr1 | 102803560 | 102804460 | 6.88E−06 | −29.1251075 | Hypo-methylated | NEPC\|CRPC-Adeno DMRs |
| chr1 | 102867728 | 102867822 | 5.17E−06 | −22.90435977 | Hypo-methylated | NEPC\|CRPC-Adeno DMRs |
| chr1 | 105460250 | 105461169 | 1.70E−06 | 23.17106696 | Hyper-methylated | NEPC\|CRPC-Adeno DMRs |
| chr1 | 105469590 | 105471363 | 0.002023694 | 24.07688209 | Hyper-methylated | NEPC\|CRPC-Adeno DMRs |
| chr1 | 105472598 | 105472739 | 2.60E−09 | 27.4558114 | Hyper-methylated | NEPC\|CRPC-Adeno DMRs |
| chr1 | 105474399 | 105474451 | 0.000549237 | 22.70290011 | Hyper-methylated | NEPC\|CRPC-Adeno DMRs |
| chr1 | 107103708 | 107104119 | 4.20E−09 | −21.36352087 | Hypo-methylated | NEPC\|CRPC-Adeno DMRs |
| chr1 | 108602916 | 108603122 | 1.71E−09 | 25.06372442 | Hyper-methylated | NEPC\|CRPC-Adeno DMRs |
| chr1 | 109523216 | 109524435 | 4.66E−05 | −28.38012914 | Hypo-methylated | NEPC\|CRPC-Adeno DMRs |
| chr1 | 110873010 | 110873170 | 0.001221669 | −26.74025341 | Hypo-methylated | NEPC\|CRPC-Adeno DMRs |
| chr1 | 111957727 | 111958255 | 0.000926684 | −45.48011174 | Hypo-methylated | NEPC\|CRPC-Adeno DMRs |
| chr1 | 113992763 | 113994167 | 8.00E−13 | 31.37393241 | Hyper-methylated | NEPC\|CRPC-Adeno DMRs |
| chr1 | 114359410 | 114360124 | 4.72E−07 | 28.17447793 | Hyper-methylated | NEPC\|CRPC-Adeno DMRs |
| chr1 | 115918770 | 115919993 | 7.64E−28 | 25.05568087 | Hyper-methylated | NEPC\|CRPC-Adeno DMRs |
| chr1 | 119599142 | 119599175 | 5.50E−05 | −27.6502924 | Hypo-methylated | NEPC\|CRPC-Adeno DMRs |
| chr1 | 119601016 | 119602645 | 9.09E−05 | −21.95368978 | Hypo-methylated | NEPC\|CRPC-Adeno DMRs |
| chr1 | 119606289 | 119606544 | 0.009167638 | 26.56572927 | Hyper-methylated | NEPC\|CRPC-Adeno DMRs |
| chr1 | 119610407 | 119610425 | 0.000303685 | 20.69532164 | Hyper-methylated | NEPC\|CRPC-Adeno DMRs |
| chr1 | 119915117 | 119915902 | 6.00E−08 | 22.09453582 | Hyper-methylated | NEPC\|CRPC-Adeno DMRs |
| chr1 | 120452147 | 120452321 | 0.000488426 | −29.53157895 | Hypo-methylated | NEPC\|CRPC-Adeno DMRs |
| chr1 | 120825589 | 120826561 | 0.005239183 | −28.72839506 | Hypo-methylated | NEPC\|CRPC-Adeno DMRs |
| chr1 | 121071218 | 121071615 | 2.14E−06 | −20.7802562 | Hypo-methylated | NEPC\|CRPC-Adeno DMRs |
| chr1 | 121153634 | 121153791 | 0.003348504 | −40.20637589 | Hypo-methylated | NEPC\|CRPC-Adeno DMRs |
| chr1 | 121200581 | 121200763 | 2.64E−09 | 21.24714515 | Hyper-methylated | NEPC\|CRPC-Adeno DMRs |
| chr1 | 121223604 | 121223892 | 7.90E−06 | −40.19098184 | Hypo-methylated | NEPC\|CRPC-Adeno DMRs |
| chr1 | 121238158 | 121244268 | 0.001549499 | 26.48457602 | Hyper-methylated | NEPC\|CRPC-Adeno DMRs |
| chr1 | 121580735 | 121585855 | 1.47E−05 | −21.33102312 | Hypo-methylated | NEPC\|CRPC-Adeno DMRs |
| chr1 | 121604037 | 121605767 | 5.50E−05 | −36.93508772 | Hypo-methylated | NEPC\|CRPC-Adeno DMRs |
| chr1 | 121644936 | 121645587 | 7.96E−07 | −33.71186475 | Hypo-methylated | NEPC\|CRPC-Adeno DMRs |
| chr1 | 121846490 | 121847867 | 0.007136365 | −25.76876566 | Hypo-methylated | NEPC\|CRPC-Adeno DMRs |
| chr1 | 128177475 | 128180539 | 0.005557404 | −21.7417154 | Hypo-methylated | NEPC\|CRPC-Adeno DMRs |
| chr1 | 128366766 | 128370333 | 0.00324027 | −20.84426907 | Hypo-methylated | NEPC\|CRPC-Adeno DMRs |
| chr1 | 128411111 | 128412003 | 0.001689936 | −22.42920649 | Hypo-methylated | NEPC\|CRPC-Adeno DMRs |
| chr1 | 128438863 | 128439315 | 2.69E−07 | −24.72315262 | Hypo-methylated | NEPC\|CRPC-Adeno DMRs |
| chr1 | 128616678 | 128616767 | 0.000549237 | −39.66474221 | Hypo-methylated | NEPC\|CRPC-Adeno DMRs |
| chr1 | 129019404 | 129019534 | 0.003525992 | −42.77777778 | Hypo-methylated | NEPC\|CRPC-Adeno DMRs |
| chr1 | 129063081 | 129063273 | 0.000581135 | −31.43997248 | Hypo-methylated | NEPC\|CRPC-Adeno DMRs |
| chr1 | 129122084 | 129123431 | 5.75E−05 | −28.03508772 | Hypo-methylated | NEPC\|CRPC-Adeno DMRs |
| chr1 | 129198482 | 129198825 | 0.004750844 | −30.19461644 | Hypo-methylated | NEPC\|CRPC-Adeno DMRs |
| chr1 | 129225176 | 129244012 | 0.00302923 | −21.93509816 | Hypo-methylated | NEPC\|CRPC-Adeno DMRs |
| chr1 | 129260154 | 129337554 | 2.33E−05 | −20.19219088 | Hypo-methylated | NEPC\|CRPC-Adeno DMRs |
| chr1 | 129352316 | 129361180 | 0.000193859 | −22.05175609 | Hypo-methylated | NEPC\|CRPC-Adeno DMRs |
| chr1 | 129435812 | 129491399 | 0.000246327 | −23.97108751 | Hypo-methylated | NEPC\|CRPC-Adeno DMRs |
| chr1 | 129528148 | 129540816 | 0.005737741 | −21.41918686 | Hypo-methylated | NEPC\|CRPC-Adeno DMRs |
| chr1 | 130076619 | 130344545 | 1.50E−07 | −34.59962519 | Hypo-methylated | NEPC\|CRPC-Adeno DMRs |
| chr2 | 130408104 | 130496813 | 8.93E−07 | −28.94061341 | Hypo-methylated | NEPC\|CRPC-Adeno DMRs |
| chr2 | 130510355 | 130515318 | 0.000101637 | −26.40830562 | Hypo-methylated | NEPC\|CRPC-Adeno DMRs |
| chr2 | 130661361 | 130671156 | 5.38E−06 | −22.21064621 | Hypo-methylated | NEPC\|CRPC-Adeno DMRs |
| chr2 | 131060249 | 131073820 | 0.000337664 | −32.73859649 | Hypo-methylated | NEPC\|CRPC-Adeno DMRs |
| chr2 | 131514052 | 131514704 | 9.21E−06 | −20.47162023 | Hypo-methylated | NEPC\|CRPC-Adeno DMRs |
| chr2 | 131936509 | 131968179 | 3.43E−06 | −22.58689426 | Hypo-methylated | NEPC\|CRPC-Adeno DMRs |
| chr2 | 132251816 | 132252133 | 0.000176134 | −29.04024338 | Hypo-methylated | NEPC\|CRPC-Adeno DMRs |
| chr2 | 132293756 | 132391658 | 4.03E−10 | −33.02168439 | Hypo-methylated | NEPC\|CRPC-Adeno DMRs |
| chr2 | 132877035 | 132944869 | 0.004750844 | −31.15037594 | Hypo-methylated | NEPC\|CRPC-Adeno DMRs |
| chr2 | 133062272 | 133062425 | 0.000200845 | 35.97508772 | Hyper-methylated | NEPC\|CRPC-Adeno DMRs |
| chr2 | 133246899 | 133247159 | 4.14E−08 | 36.7942792 | Hyper-methylated | NEPC\|CRPC-Adeno DMRs |
| chr2 | 134785429 | 134785660 | 0.001012154 | 25.96165414 | Hyper-methylated | NEPC\|CRPC-Adeno DMRs |
| chr2 | 143460125 | 144172370 | 4.88E−06 | −25.82494081 | Hypo-methylated | NEPC\|CRPC-Adeno DMRs |
| chr2 | 153192734 | 153192875 | 1.22E−05 | −20.88712682 | Hypo-methylated | NEPC\|CRPC-Adeno DMRs |
| chr2 | 161052000 | 161125859 | 0.008916498 | −24.86994901 | Hypo-methylated | NEPC\|CRPC-Adeno DMRs |
| chr2 | 162838347 | 162838416 | 0.001694183 | −25.95238095 | Hypo-methylated | NEPC\|CRPC-Adeno DMRs |
| chr2 | 163299224 | 163622381 | 0.005456645 | 21.47698413 | Hyper-methylated | NEPC\|CRPC-Adeno DMRs |
| chr2 | 163882749 | 163883094 | 1.70E−05 | 22.68208333 | Hyper-methylated | NEPC\|CRPC-Adeno DMRs |
| chr2 | 170219995 | 170220470 | 2.03E−06 | −20.49118095 | Hypo-methylated | NEPC\|CRPC-Adeno DMRs |
| chr2 | 175205481 | 175205751 | 2.71E−06 | 20.63542189 | Hyper-methylated | NEPC\|CRPC-Adeno DMRs |
| chr2 | 179278075 | 179278593 | 8.97E−08 | −21.46412787 | Hypo-methylated | NEPC\|CRPC-Adeno DMRs |
| chr2 | 182546475 | 182547973 | 1.80E−07 | 20.57561404 | Hyper-methylated | NEPC\|CRPC-Adeno DMRs |
| chr2 | 186603546 | 186603618 | 0.000549237 | 24.79033298 | Hyper-methylated | NEPC\|CRPC-Adeno DMRs |
| chr2 | 188733953 | 188734511 | 0.009274146 | −27.73866959 | Hypo-methylated | NEPC\|CRPC-Adeno DMRs |
| chr2 | 197379889 | 197380122 | 0.000176134 | −42.02678019 | Hypo-methylated | NEPC\|CRPC-Adeno DMRs |
| chr2 | 198315420 | 198315486 | 0.000549237 | −25.87819549 | Hypo-methylated | NEPC\|CRPC-Adeno DMRs |
| chr2 | 206547479 | 206547524 | 0.000192248 | −21.66059942 | Hypo-methylated | NEPC\|CRPC-Adeno DMRs |
| chr2 | 207115220 | 207115508 | 0.000291618 | 20.64307992 | Hyper-methylated | NEPC\|CRPC-Adeno DMRs |
| chr2 | 207521644 | 207563209 | 0.004750844 | −20.70701754 | Hypo-methylated | NEPC\|CRPC-Adeno DMRs |
| chr2 | 207929328 | 207972442 | 0.000176134 | −49.82702851 | Hypo-methylated | NEPC\|CRPC-Adeno DMRs |
| chr2 | 217850623 | 217850810 | 0.001694183 | −33.93518519 | Hypo-methylated | NEPC\|CRPC-Adeno DMRs |
| chr2 | 219748326 | 219748628 | 5.23E−13 | −23.12146814 | Hypo-methylated | NEPC\|CRPC-Adeno DMRs |
| chr2 | 219749016 | 219749103 | 0.005737741 | −26.24414775 | Hypo-methylated | NEPC\|CRPC-Adeno DMRs |
| chr2 | 220270066 | 220283866 | 3.22E−16 | 25.12400617 | Hyper-methylated | NEPC\|CRPC-Adeno DMRs |
| chr2 | 220316324 | 220317803 | 0.000102271 | −22.15006579 | Hypo-methylated | NEPC\|CRPC-Adeno DMRs |
| chr2 | 220397136 | 220397259 | 5.75E−05 | −29.27138397 | Hypo-methylated | NEPC\|CRPC-Adeno DMRs |
| chr2 | 220439493 | 220439633 | 1.70E−05 | −27.35157895 | Hypo-methylated | NEPC\|CRPC-Adeno DMRs |
| chr2 | 220509463 | 220509832 | 2.85E−10 | −45.58795322 | Hypo-methylated | NEPC\|CRPC-Adeno DMRs |
| chr2 | 222826407 | 222826513 | 0.000176134 | −32.00328947 | Hypo-methylated | NEPC\|CRPC-Adeno DMRs |
| chr2 | 224477834 | 224478322 | 5.01E−05 | −24.43060429 | Hypo-methylated | NEPC\|CRPC-Adeno DMRs |
| chr2 | 227898189 | 227898378 | 0.000549237 | 24.99906752 | Hyper-methylated | NEPC\|CRPC-Adeno DMRs |
| chr2 | 232290776 | 232291013 | 0.003932932 | −27.77493502 | Hypo-methylated | NEPC\|CRPC-Adeno DMRs |
| chr2 | 232536414 | 232545414 | 2.22E−11 | −47.43532003 | Hypo-methylated | NEPC\|CRPC-Adeno DMRs |
| chr2 | 233306667 | 233306902 | 0.009458442 | −25.75048733 | Hypo-methylated | NEPC\|CRPC-Adeno DMRs |
| chr2 | 233464792 | 233464920 | 0.009458442 | −23.64473684 | Hypo-methylated | NEPC\|CRPC-Adeno DMRs |
| chr2 | 233888552 | 233916226 | 5.61E−05 | −20.08187749 | Hypo-methylated | NEPC\|CRPC-Adeno DMRs |
| chr2 | 234079048 | 234083610 | 0.000153872 | −21.05410401 | Hypo-methylated | NEPC\|CRPC-Adeno DMRs |
| chr2 | 234699500 | 234704770 | 4.00E−06 | −24.53621233 | Hypo-methylated | NEPC\|CRPC-Adeno DMRs |
| chr2 | 235237250 | 235237950 | 0.001694183 | −34.99074074 | Hypo-methylated | NEPC\|CRPC-Adeno DMRs |
| chr2 | 235737595 | 235740118 | 0.000716333 | −24.57805011 | Hypo-methylated | NEPC\|CRPC-Adeno DMRs |
| chr2 | 235941822 | 235949479 | 0.000104758 | −27.16893303 | Hypo-methylated | NEPC\|CRPC-Adeno DMRs |
| chr2 | 236486227 | 236486417 | 0.000549237 | −38.75005669 | Hypo-methylated | NEPC\|CRPC-Adeno DMRs |
| chr2 | 236528752 | 236529012 | 0.002927544 | −25.12469136 | Hypo-methylated | NEPC\|CRPC-Adeno DMRs |
| chr2 | 237501472 | 237501628 | 0.001694183 | −31.77022059 | Hypo-methylated | NEPC\|CRPC-Adeno DMRs |
| chr2 | 237727216 | 237727409 | 0.001694183 | 28.49338624 | Hyper-methylated | NEPC\|CRPC-Adeno DMRs |
| chr2 | 238404105 | 238404446 | 1.70E−05 | 24.31431373 | Hyper-methylated | NEPC\|CRPC-Adeno DMRs |
| chr2 | 238472648 | 238472726 | 0.003327791 | 21.96388889 | Hyper-methylated | NEPC\|CRPC-Adeno DMRs |
| chr2 | 238610221 | 238612278 | 0.000549237 | −28.16165633 | Hypo-methylated | NEPC\|CRPC-Adeno DMRs |
| chr2 | 238794510 | 238794838 | 4.65E−07 | −38.12277526 | Hypo-methylated | NEPC\|CRPC-Adeno DMRs |
| chr2 | 238832403 | 238832515 | 6.03E−06 | 27.51333333 | Hyper-methylated | NEPC\|CRPC-Adeno DMRs |
| chr2 | 238856011 | 238865307 | 1.73E−08 | 34.37750043 | Hyper-methylated | NEPC\|CRPC-Adeno DMRs |
| chr2 | 239039611 | 239040086 | 1.41E−08 | −27.08506006 | Hypo-methylated | NEPC\|CRPC-Adeno DMRs |
| chr2 | 239048258 | 239048449 | 2.07E−08 | 30.3332265 | Hyper-methylated | NEPC\|CRPC-Adeno DMRs |
| chr2 | 239066848 | 239067031 | 3.15E−05 | −26.96352941 | Hypo-methylated | NEPC\|CRPC-Adeno DMRs |
| chr2 | 239129499 | 239130134 | 9.74E−06 | −27.34281674 | Hypo-methylated | NEPC\|CRPC-Adeno DMRs |
| chr2 | 239147433 | 239148142 | 5.07E−08 | −24.47076114 | Hypo-methylated | NEPC\|CRPC-Adeno DMRs |
| chr2 | 239149756 | 239149938 | 0.000553171 | −20.06018519 | Hypo-methylated | NEPC\|CRPC-Adeno DMRs |
| chr2 | 239323913 | 239324211 | 0.003113626 | −38.39738173 | Hypo-methylated | NEPC\|CRPC-Adeno DMRs |
| chr2 | 239413150 | 239413386 | 3.06E−06 | −37.48166667 | Hypo-methylated | NEPC\|CRPC-Adeno DMRs |
| chr2 | 239453395 | 239467821 | 0.000104758 | −32.08421717 | Hypo-methylated | NEPC\|CRPC-Adeno DMRs |
| chr2 | 239487105 | 239612308 | 6.61E−09 | −20.68710654 | Hypo-methylated | NEPC\|CRPC-Adeno DMRs |
| chr2 | 239756501 | 239757623 | 6.64E−11 | 26.80913042 | Hyper-methylated | NEPC\|CRPC-Adeno DMRs |
| chr2 | 239780522 | 239800796 | 0.000218789 | −21.59291472 | Hypo-methylated | NEPC\|CRPC-Adeno DMRs |
| chr2 | 239872281 | 239907659 | 3.16E−06 | −22.67556307 | Hypo-methylated | NEPC\|CRPC-Adeno DMRs |
| chr2 | 239907717 | 239907900 | 1.40E−06 | −25.37291667 | Hypo-methylated | NEPC\|CRPC-Adeno DMRs |
| chr2 | 239907945 | 239908470 | 0.000184724 | −21.88547308 | Hypo-methylated | NEPC\|CRPC-Adeno DMRs |
| chr2 | 240002663 | 240003506 | 2.08E−05 | −20.58040936 | Hypo-methylated | NEPC\|CRPC-Adeno DMRs |
| chr2 | 240044530 | 240044797 | 0.000303685 | −25.20130719 | Hypo-methylated | NEPC\|CRPC-Adeno DMRs |
| chr2 | 240271689 | 240271786 | 0.001694183 | −27.36111111 | Hypo-methylated | NEPC\|CRPC-Adeno DMRs |
| chr2 | 240386993 | 240387067 | 0.000832692 | −29.325 | Hypo-methylated | NEPC\|CRPC-Adeno DMRs |
| chr2 | 241176159 | 241185506 | 1.07E−06 | −25.27123216 | Hypo-methylated | NEPC\|CRPC-Adeno DMRs |
| chr2 | 241232629 | 241233216 | 1.41E−05 | −50.57723797 | Hypo-methylated | NEPC\|CRPC-Adeno DMRs |
| chr2 | 241393816 | 241397544 | 1.00E−12 | 38.18395326 | Hyper-methylated | NEPC\|CRPC-Adeno DMRs |
| chr2 | 241408285 | 241408720 | 0.000440683 | −24.19166965 | Hypo-methylated | NEPC\|CRPC-Adeno DMRs |
| chr2 | 241772069 | 241772103 | 0.003525992 | −27.11111111 | Hypo-methylated | NEPC\|CRPC-Adeno DMRs |
| chr2 | 241830975 | 241831203 | 0.000549237 | −31.47376775 | Hypo-methylated | NEPC\|CRPC-Adeno DMRs |
| chr2 | 242139533 | 242139616 | 0.000581135 | 34.62828947 | Hyper-methylated | NEPC\|CRPC-Adeno DMRs |
| chr2 | 242355688 | 242356453 | 0.000711935 | −27.31578947 | Hypo-methylated | NEPC\|CRPC-Adeno DMRs |
| chr2 | 242481481 | 242481937 | 2.41E−07 | −30.10079745 | Hypo-methylated | NEPC\|CRPC-Adeno DMRs |
| chr2 | 242812420 | 242812661 | 1.57E−06 | −25.60897266 | Hypo-methylated | NEPC\|CRPC-Adeno DMRs |
| chr3 | 463049 | 513460 | 0.001694183 | −43.68333333 | Hypo-methylated | NEPC\|CRPC-Adeno DMRs |
| chr3 | 577629 | 577844 | 3.06E−06 | 26.15192224 | Hyper-methylated | NEPC\|CRPC-Adeno DMRs |
| chr3 | 2125621 | 2125739 | 0.001694183 | −29.78289474 | Hypo-methylated | NEPC\|CRPC-Adeno DMRs |
| chr3 | 2211250 | 2395440 | 2.96E−08 | −24.9629741 | Hypo-methylated | NEPC\|CRPC-Adeno DMRs |
| chr3 | 2547773 | 2771394 | 0.000154411 | −25.14076933 | Hypo-methylated | NEPC\|CRPC-Adeno DMRs |
| chr3 | 2846718 | 3126422 | 4.31E−10 | −23.08686935 | Hypo-methylated | NEPC\|CRPC-Adeno DMRs |
| chr3 | 3222976 | 3761172 | 1.85E−05 | −24.72503578 | Hypo-methylated | NEPC\|CRPC-Adeno DMRs |
| chr3 | 4061898 | 4287901 | 4.74E−05 | −29.32643737 | Hypo-methylated | NEPC\|CRPC-Adeno DMRs |
| chr3 | 4650559 | 4701228 | 0.001980589 | −21.43587891 | Hypo-methylated | NEPC\|CRPC-Adeno DMRs |
| chr3 | 9595319 | 9595668 | 2.46E−06 | 21.1862008 | Hyper-methylated | NEPC\|CRPC-Adeno DMRs |
| chr3 | 9957450 | 9957637 | 1.77E−06 | −23.56558062 | Hypo-methylated | NEPC\|CRPC-Adeno DMRs |
| chr3 | 10363228 | 10363292 | 0.00222033 | 23.30555556 | Hyper-methylated | NEPC\|CRPC-Adeno DMRs |
| chr3 | 10877364 | 10928399 | 6.82E−05 | −30.43403872 | Hypo-methylated | NEPC\|CRPC-Adeno DMRs |
| chr3 | 11046887 | 11072823 | 2.68E−07 | −26.94088074 | Hypo-methylated | NEPC\|CRPC-Adeno DMRs |
| chr3 | 11112978 | 11133754 | 2.64E−05 | −23.11886152 | Hypo-methylated | NEPC\|CRPC-Adeno DMRs |
| chr3 | 11138548 | 11168621 | 2.58E−05 | −25.26545015 | Hypo-methylated | NEPC\|CRPC-Adeno DMRs |
| chr3 | 12672954 | 12673023 | 0.000549237 | −43.48517126 | Hypo-methylated | NEPC\|CRPC-Adeno DMRs |
| chr3 | 13008783 | 13009257 | 2.76E−21 | 35.76399768 | Hyper-methylated | NEPC\|CRPC-Adeno DMRs |
| chr3 | 13060941 | 13069923 | 2.23E−06 | −28.9748894 | Hypo-methylated | NEPC\|CRPC-Adeno DMRs |
| chr3 | 13114303 | 13114344 | 0.003113626 | −21.60350877 | Hypo-methylated | NEPC\|CRPC-Adeno DMRs |
| chr3 | 13245569 | 13246093 | 2.81E−06 | 32.3879257 | Hyper-methylated | NEPC\|CRPC-Adeno DMRs |
| chr3 | 13367277 | 13367357 | 0.000549237 | −22.96616541 | Hypo-methylated | NEPC\|CRPC-Adeno DMRs |
| chr3 | 13560180 | 13569838 | 0.007136365 | −29.05864662 | Hypo-methylated | NEPC\|CRPC-Adeno DMRs |
| chr3 | 14445043 | 14445162 | 0.001694183 | −27.02280702 | Hypo-methylated | NEPC\|CRPC-Adeno DMRs |
| chr3 | 14556022 | 14559856 | 0.000953892 | −28.0063274 | Hypo-methylated | NEPC\|CRPC-Adeno DMRs |
| chr3 | 14806535 | 14846534 | 1.53E−09 | −22.37547218 | Hypo-methylated | NEPC\|CRPC-Adeno DMRs |
| chr3 | 14877771 | 14910141 | 0.000224358 | −24.82001462 | Hypo-methylated | NEPC\|CRPC-Adeno DMRs |
| chr3 | 15160267 | 15204210 | 0.000538714 | 22.84587069 | Hyper-methylated | NEPC\|CRPC-Adeno DMRs |
| chr3 | 18485142 | 18485766 | 9.36E−16 | −22.70118134 | Hypo-methylated | NEPC\|CRPC-Adeno DMRs |
| chr3 | 22413885 | 22414340 | 3.82E−17 | 35.11866472 | Hyper-methylated | NEPC\|CRPC-Adeno DMRs |
| chr3 | 24780720 | 24855565 | 0.000542154 | −22.05241893 | Hypo-methylated | NEPC\|CRPC-Adeno DMRs |
| chr3 | 25109412 | 25348735 | 1.11E−08 | −22.19636591 | Hypo-methylated | NEPC\|CRPC-Adeno DMRs |
| chr3 | 25928241 | 26608348 | 6.92E−10 | −31.8026437 | Hypo-methylated | NEPC\|CRPC-Adeno DMRs |
| chr3 | 26664116 | 26665533 | 7.94E−17 | 33.53493626 | Hyper-methylated | NEPC\|CRPC-Adeno DMRs |
| chr3 | 26666972 | 27168486 | 3.44E−06 | −27.71704134 | Hypo-methylated | NEPC\|CRPC-Adeno DMRs |
| chr3 | 27944115 | 28241455 | 0.002692265 | −21.07048872 | Hypo-methylated | NEPC\|CRPC-Adeno DMRs |
| chr3 | 29185724 | 29221824 | 0.000168572 | −20.60233811 | Hypo-methylated | NEPC\|CRPC-Adeno DMRs |
| chr3 | 38070605 | 38070935 | 1.85E−05 | 22.99612442 | Hyper-methylated | NEPC\|CRPC-Adeno DMRs |
| chr3 | 38583417 | 38584178 | 0.001545937 | −30.12396733 | Hypo-methylated | NEPC\|CRPC-Adeno DMRs |
| chr3 | 42304914 | 42305307 | 1.54E−10 | −24.6642161 | Hypo-methylated | NEPC\|CRPC-Adeno DMRs |
| chr3 | 42743744 | 42743829 | 0.007136365 | −20.08195489 | Hypo-methylated | NEPC\|CRPC-Adeno DMRs |
| chr3 | 46274813 | 46394598 | 5.23E−05 | −24.18838306 | Hypo-methylated | NEPC\|CRPC-Adeno DMRs |
| chr3 | 48690417 | 48694482 | 2.85E−16 | 24.86272825 | Hyper-methylated | NEPC\|CRPC-Adeno DMRs |
| chr3 | 48698457 | 48699857 | 6.62E−07 | 26.74565037 | Hyper-methylated | NEPC\|CRPC-Adeno DMRs |
| chr3 | 49381502 | 49383217 | 0.002094266 | 21.87965011 | Hyper-methylated | NEPC\|CRPC-Adeno DMRs |
| chr3 | 49692360 | 49695184 | 0.000721524 | −36.80171583 | Hypo-methylated | NEPC\|CRPC-Adeno DMRs |
| chr3 | 49940117 | 49941426 | 5.70E−05 | −29.68770968 | Hypo-methylated | NEPC\|CRPC-Adeno DMRs |
| chr3 | 50359583 | 50360007 | 2.22E−11 | −36.41238882 | Hypo-methylated | NEPC\|CRPC-Adeno DMRs |
| chr3 | 50402058 | 50402563 | 0.006339783 | 21.59683236 | Hyper-methylated | NEPC\|CRPC-Adeno DMRs |
| chr3 | 50480513 | 50480616 | 0.001694183 | −55.54561404 | Hypo-methylated | NEPC\|CRPC-Adeno DMRs |
| chr3 | 50502527 | 50502597 | 0.001694183 | −34.05877193 | Hypo-methylated | NEPC\|CRPC-Adeno DMRs |
| chr3 | 50795465 | 51221599 | 2.36E−08 | −27.51373467 | Hypo-methylated | NEPC\|CRPC-Adeno DMRs |
| chr3 | 51298580 | 51378785 | 0.002174767 | −25.00891898 | Hypo-methylated | NEPC\|CRPC-Adeno DMRs |
| chr3 | 51489993 | 51509734 | 0.000549237 | −21.61428571 | Hypo-methylated | NEPC\|CRPC-Adeno DMRs |
| chr3 | 51818199 | 51853767 | 0.007136365 | −30.72330827 | Hypo-methylated | NEPC\|CRPC-Adeno DMRs |
| chr3 | 51884494 | 51944246 | 0.000549237 | −22.36215626 | Hypo-methylated | NEPC\|CRPC-Adeno DMRs |
| chr3 | 52402672 | 52403333 | 2.17E−07 | −30.96656347 | Hypo-methylated | NEPC\|CRPC-Adeno DMRs |
| chr3 | 52537803 | 52537879 | 5.50E−05 | −39.98304094 | Hypo-methylated | NEPC\|CRPC-Adeno DMRs |
| chr3 | 53837301 | 53837653 | 1.55E−06 | −45.31413255 | Hypo-methylated | NEPC\|CRPC-Adeno DMRs |
| chr3 | 61280786 | 61280835 | 0.000549237 | 29.31353383 | Hyper-methylated | NEPC\|CRPC-Adeno DMRs |
| chr3 | 66002883 | 66002980 | 0.000832692 | −44.35584795 | Hypo-methylated | NEPC\|CRPC-Adeno DMRs |
| chr3 | 68056641 | 68057167 | 0.000337664 | 24.25637427 | Hyper-methylated | NEPC\|CRPC-Adeno DMRs |
| chr3 | 71194772 | 71213646 | 0.001249757 | 38.60052651 | Hyper-methylated | NEPC\|CRPC-Adeno DMRs |
| chr3 | 72149779 | 72149976 | 0.000549237 | −25.11597973 | Hypo-methylated | NEPC\|CRPC-Adeno DMRs |
| chr3 | 72305843 | 72308226 | 0.000176134 | −40.72369281 | Hypo-methylated | NEPC\|CRPC-Adeno DMRs |
| chr3 | 74420028 | 74601215 | 0.000561687 | −20.24012346 | Hypo-methylated | NEPC\|CRPC-Adeno DMRs |
| chr3 | 74819415 | 75002211 | 0.001847465 | −20.22825922 | Hypo-methylated | NEPC\|CRPC-Adeno DMRs |
| chr3 | 75668646 | 75668686 | 0.000224358 | 35.78859127 | Hyper-methylated | NEPC\|CRPC-Adeno DMRs |
| chr3 | 75683144 | 75697750 | 0.008187764 | −27.61354724 | Hypo-methylated | NEPC\|CRPC-Adeno DMRs |
| chr3 | 75707580 | 75707707 | 1.43E−05 | 25.20830688 | Hyper-methylated | NEPC\|CRPC-Adeno DMRs |
| chr3 | 75707805 | 75707826 | 0.001694183 | 20.24126984 | Hyper-methylated | NEPC\|CRPC-Adeno DMRs |
| chr3 | 75721563 | 75721594 | 0.001249757 | 28.05324722 | Hyper-methylated | NEPC\|CRPC-Adeno DMRs |
| chr3 | 87042004 | 87091725 | 0.001549499 | −26.87361111 | Hypo-methylated | NEPC\|CRPC-Adeno DMRs |
| chr3 | 87424528 | 87728434 | 2.36E−10 | −31.3923574 | Hypo-methylated | NEPC\|CRPC-Adeno DMRs |
| chr3 | 87891471 | 88036020 | 4.03E−08 | −29.81320106 | Hypo-methylated | NEPC\|CRPC-Adeno DMRs |
| chr3 | 94024788 | 94656908 | 2.08E−10 | −28.72438824 | Hypo-methylated | NEPC\|CRPC-Adeno DMRs |
| chr3 | 94665363 | 95313928 | 1.73E−11 | −25.90864732 | Hypo-methylated | NEPC\|CRPC-Adeno DMRs |
| chr3 | 107833652 | 107872310 | 0.003525992 | 21.93820663 | Hyper-methylated | NEPC\|CRPC-Adeno DMRs |
| chr3 | 111431503 | 111469563 | 0.000549237 | −49.08404221 | Hypo-methylated | NEPC\|CRPC-Adeno DMRs |
| chr3 | 113417992 | 113418339 | 0.000184724 | 26.3654321 | Hyper-methylated | NEPC\|CRPC-Adeno DMRs |
| chr3 | 115133824 | 115332585 | 0.000153872 | 21.5758302 | Hyper-methylated | NEPC\|CRPC-Adeno DMRs |
| chr3 | 119528807 | 119529068 | 3.68E−07 | −23.09808493 | Hypo-methylated | NEPC\|CRPC-Adeno DMRs |
| chr3 | 123003270 | 123003529 | 0.002859601 | −24.85842105 | Hypo-methylated | NEPC\|CRPC-Adeno DMRs |
| chr3 | 123166795 | 123166905 | 1.09E−08 | 36.64989731 | Hyper-methylated | NEPC\|CRPC-Adeno DMRs |
| chr3 | 123420221 | 123420466 | 0.000549237 | 27.37769424 | Hyper-methylated | NEPC\|CRPC-Adeno DMRs |
| chr3 | 125677175 | 125677258 | 6.03E−06 | −28.87922379 | Hypo-methylated | NEPC\|CRPC-Adeno DMRs |
| chr3 | 126397531 | 126397741 | 0.007136365 | −38.14205182 | Hypo-methylated | NEPC\|CRPC-Adeno DMRs |
| chr3 | 126714171 | 126715234 | 0.000542154 | 20.40382775 | Hyper-methylated | NEPC\|CRPC-Adeno DMRs |
| chr3 | 126716223 | 126718487 | 0.004222625 | −24.42193171 | Hypo-methylated | NEPC\|CRPC-Adeno DMRs |
| chr3 | 127056919 | 127057150 | 2.62E−13 | 24.48829205 | Hyper-methylated | NEPC\|CRPC-Adeno DMRs |
| chr3 | 127318195 | 127318252 | 1.70E−05 | −31.73754386 | Hypo-methylated | NEPC\|CRPC-Adeno DMRs |
| chr3 | 128140328 | 128141587 | 6.06E−05 | 31.58228906 | Hyper-methylated | NEPC\|CRPC-Adeno DMRs |
| chr3 | 128209516 | 128211155 | 7.02E−16 | 23.13433595 | Hyper-methylated | NEPC\|CRPC-Adeno DMRs |
| chr3 | 128564943 | 128565094 | 0.003525992 | 25.81143791 | Hyper-methylated | NEPC\|CRPC-Adeno DMRs |
| chr3 | 128653931 | 128658829 | 0.009458442 | −23.43259569 | Hypo-methylated | NEPC\|CRPC-Adeno DMRs |
| chr3 | 128787249 | 128787525 | 0.000176134 | 22.60641161 | Hyper-methylated | NEPC\|CRPC-Adeno DMRs |
| chr3 | 129299973 | 129300113 | 0.003113626 | −23.35964912 | Hypo-methylated | NEPC\|CRPC-Adeno DMRs |
| chr3 | 131753881 | 131753954 | 2.46E−05 | 42.16437247 | Hyper-methylated | NEPC\|CRPC-Adeno DMRs |
| chr3 | 137501042 | 137626636 | 0.001694183 | −22.18415831 | Hypo-methylated | NEPC\|CRPC-Adeno DMRs |
| chr3 | 138765942 | 138825790 | 8.71E−07 | −36.06678363 | Hypo-methylated | NEPC\|CRPC-Adeno DMRs |
| chr3 | 138904612 | 139053137 | 7.90E−08 | −28.08957955 | Hypo-methylated | NEPC\|CRPC-Adeno DMRs |
| chr3 | 139379620 | 139396853 | 8.52E−17 | −21.95038986 | Hypo-methylated | NEPC\|CRPC-Adeno DMRs |
| chr3 | 140814064 | 140814119 | 0.001694183 | −44.70272904 | Hypo-methylated | NEPC\|CRPC-Adeno DMRs |
| chr3 | 143130737 | 143292991 | 0.000209051 | 26.59270335 | Hyper-methylated | NEPC\|CRPC-Adeno DMRs |
| chr3 | 146187388 | 146188416 | 8.97E−08 | −20.11957467 | Hypo-methylated | NEPC\|CRPC-Adeno DMRs |
| chr3 | 146872122 | 146872206 | 0.001694183 | −20.10057261 | Hypo-methylated | NEPC\|CRPC-Adeno DMRs |
| chr3 | 147126697 | 147129334 | 5.56E−28 | 22.61330125 | Hyper-methylated | NEPC\|CRPC-Adeno DMRs |
| chr3 | 148040232 | 148335993 | 3.46E−05 | −23.96160533 | Hypo-methylated | NEPC\|CRPC-Adeno DMRs |
| chr3 | 150675538 | 150696776 | 0.004750844 | −22.97752193 | Hypo-methylated | NEPC\|CRPC-Adeno DMRs |
| chr3 | 151158578 | 151379420 | 0.0001635 | −25.03709511 | Hypo-methylated | NEPC\|CRPC-Adeno DMRs |
| chr3 | 151523968 | 151525465 | 0.000484821 | −33.37719089 | Hypo-methylated | NEPC\|CRPC-Adeno DMRs |
| chr3 | 152277188 | 152352045 | 4.43E−06 | −23.09415461 | Hypo-methylated | NEPC\|CRPC-Adeno DMRs |
| chr3 | 152927587 | 153356991 | 6.83E−14 | −22.6334674 | Hypo-methylated | NEPC\|CRPC-Adeno DMRs |
| chr3 | 153403846 | 153563964 | 1.56E−05 | −30.3546326 | Hypo-methylated | NEPC\|CRPC-Adeno DMRs |
| chr3 | 153641300 | 153784041 | 0.000220189 | −30.0825597 | Hypo-methylated | NEPC\|CRPC-Adeno DMRs |
| chr3 | 154085753 | 154087701 | 0.000101637 | −35.56562703 | Hypo-methylated | NEPC\|CRPC-Adeno DMRs |
| chr3 | 154157310 | 154337500 | 7.30E−06 | −28.766122 | Hypo-methylated | NEPC\|CRPC-Adeno DMRs |
| chr3 | 154375244 | 154402395 | 0.001012154 | −32.22529854 | Hypo-methylated | NEPC\|CRPC-Adeno DMRs |
| chr3 | 155463652 | 155463812 | 0.000100851 | −20.62018912 | Hypo-methylated | NEPC\|CRPC-Adeno DMRs |
| chr3 | 156823183 | 156827612 | 0.001549499 | −32.36907895 | Hypo-methylated | NEPC\|CRPC-Adeno DMRs |
| chr3 | 160822530 | 160823121 | 2.31E−19 | −22.30868407 | Hypo-methylated | NEPC\|CRPC-Adeno DMRs |
| chr3 | 163719850 | 163722226 | 3.13E−05 | 20.42541845 | Hyper-methylated | NEPC\|CRPC-Adeno DMRs |
| chr3 | 169529889 | 169542956 | 1.02E−07 | −22.10761543 | Hypo-methylated | NEPC\|CRPC-Adeno DMRs |
| chr3 | 170143047 | 170143637 | 0.000101637 | −34.83489279 | Hypo-methylated | NEPC\|CRPC-Adeno DMRs |
| chr3 | 170303534 | 170303701 | 0.001345309 | 21.21021853 | Hyper-methylated | NEPC\|CRPC-Adeno DMRs |
| chr3 | 179754224 | 179755087 | 2.04E−27 | 20.14397085 | Hyper-methylated | NEPC\|CRPC-Adeno DMRs |
| chr3 | 180806076 | 180901040 | 0.004750844 | −22.86944444 | Hypo-methylated | NEPC\|CRPC-Adeno DMRs |
| chr3 | 181443544 | 181444666 | 0.004750844 | 26.24013158 | Hyper-methylated | NEPC\|CRPC-Adeno DMRs |
| chr3 | 181445726 | 181445848 | 0.001694183 | −23.33333333 | Hypo-methylated | NEPC\|CRPC-Adeno DMRs |
| chr3 | 182832766 | 182849284 | 0.000581135 | −31.90026714 | Hypo-methylated | NEPC\|CRPC-Adeno DMRs |
| chr3 | 184243296 | 184243766 | 6.36E−10 | −25.23778195 | Hypo-methylated | NEPC\|CRPC-Adeno DMRs |
| chr3 | 184387338 | 184387555 | 0.005737741 | 23.92660245 | Hyper-methylated | NEPC\|CRPC-Adeno DMRs |
| chr3 | 184390859 | 184391068 | 0.00222033 | −22.61023392 | Hypo-methylated | NEPC\|CRPC-Adeno DMRs |
| chr3 | 184468640 | 184468809 | 0.002219003 | −22.76349903 | Hypo-methylated | NEPC\|CRPC-Adeno DMRs |
| chr3 | 185967142 | 185974096 | 2.78E−05 | −32.21808754 | Hypo-methylated | NEPC\|CRPC-Adeno DMRs |
| chr3 | 186803480 | 186822011 | 0.001694183 | −22.74025341 | Hypo-methylated | NEPC\|CRPC-Adeno DMRs |
| chr3 | 187455621 | 187456337 | 8.94E−07 | 22.32549402 | Hyper-methylated | NEPC\|CRPC-Adeno DMRs |
| chr3 | 192232381 | 192233064 | 6.75E−17 | 24.99494642 | Hyper-methylated | NEPC\|CRPC-Adeno DMRs |
| chr3 | 192958797 | 192959334 | 6.14E−18 | 26.90095889 | Hyper-methylated | NEPC\|CRPC-Adeno DMRs |
| chr3 | 194004633 | 194012571 | 0.00217795 | −23.094957 | Hypo-methylated | NEPC\|CRPC-Adeno DMRs |
| chr3 | 195535764 | 195537532 | 1.49E−07 | −23.34058058 | Hypo-methylated | NEPC\|CRPC-Adeno DMRs |
| chr3 | 195540912 | 195541268 | 0.003327791 | −31.36421784 | Hypo-methylated | NEPC\|CRPC-Adeno DMRs |
| chr3 | 195943487 | 195944155 | 0.005557404 | 20.61833812 | Hyper-methylated | NEPC\|CRPC-Adeno DMRs |
| chr3 | 196756053 | 196756225 | 0.000488426 | 23.03403002 | Hyper-methylated | NEPC\|CRPC-Adeno DMRs |
| chr3 | 196763256 | 196763582 | 1.48E−06 | −21.12447712 | Hypo-methylated | NEPC\|CRPC-Adeno DMRs |
| chr3 | 197183430 | 197183678 | 9.45E−11 | −20.69183007 | Hypo-methylated | NEPC\|CRPC-Adeno DMRs |
| chr3 | 197401863 | 197401996 | 0.000549237 | −30.25331466 | Hypo-methylated | NEPC\|CRPC-Adeno DMRs |
| chr3 | 197478835 | 197478908 | 0.001694183 | −26.56851852 | Hypo-methylated | NEPC\|CRPC-Adeno DMRs |
| chr4 | 107043 | 107915 | 2.73E−26 | 26.96540576 | Hyper-methylated | NEPC\|CRPC-Adeno DMRs |
| chr4 | 206385 | 207594 | 1.76E−14 | 33.35279909 | Hyper-methylated | NEPC\|CRPC-Adeno DMRs |
| chr4 | 298777 | 299047 | 2.76E−10 | 29.60379963 | Hyper-methylated | NEPC\|CRPC-Adeno DMRs |
| chr4 | 568636 | 568670 | 0.000549237 | 23.60977444 | Hyper-methylated | NEPC\|CRPC-Adeno DMRs |
| chr4 | 568722 | 568943 | 1.52E−10 | 26.39615961 | Hyper-methylated | NEPC\|CRPC-Adeno DMRs |
| chr4 | 640366 | 640542 | 1.18E−06 | 20.53181818 | Hyper-methylated | NEPC\|CRPC-Adeno DMRs |
| chr4 | 645310 | 645516 | 0.001549499 | −22.25473684 | Hypo-methylated | NEPC\|CRPC-Adeno DMRs |
| chr4 | 681086 | 681321 | 0.000151874 | 33.80914407 | Hyper-methylated | NEPC\|CRPC-Adeno DMRs |
| chr4 | 692357 | 692478 | 0.001694183 | −42.88430799 | Hypo-methylated | NEPC\|CRPC-Adeno DMRs |
| chr4 | 956594 | 956985 | 1.09E−08 | −43.20475648 | Hypo-methylated | NEPC\|CRPC-Adeno DMRs |
| chr4 | 985827 | 987836 | 2.02E−07 | 39.15922807 | Hyper-methylated | NEPC\|CRPC-Adeno DMRs |
| chr4 | 1016193 | 1016726 | 2.25E−12 | −34.95869106 | Hypo-methylated | NEPC\|CRPC-Adeno DMRs |
| chr4 | 1022723 | 1022833 | 0.000101637 | −20.84780033 | Hypo-methylated | NEPC\|CRPC-Adeno DMRs |
| chr4 | 1187895 | 1189161 | 2.21E−15 | −31.63918454 | Hypo-methylated | NEPC\|CRPC-Adeno DMRs |
| chr4 | 1338704 | 1338766 | 0.003525992 | 40.58947368 | Hyper-methylated | NEPC\|CRPC-Adeno DMRs |
| chr4 | 1398537 | 1398599 | 0.001694183 | 27.86432749 | Hyper-methylated | NEPC\|CRPC-Adeno DMRs |
| chr4 | 1498090 | 1505299 | 0.001802623 | −21.39334289 | Hypo-methylated | NEPC\|CRPC-Adeno DMRs |
| chr4 | 1558319 | 1562804 | 5.04E−06 | −20.70367537 | Hypo-methylated | NEPC\|CRPC-Adeno DMRs |
| chr4 | 1624271 | 1625485 | 7.43E−14 | −25.61002803 | Hypo-methylated | NEPC\|CRPC-Adeno DMRs |
| chr4 | 1774883 | 1775124 | 1.70E−05 | −37.59871345 | Hypo-methylated | NEPC\|CRPC-Adeno DMRs |
| chr4 | 1800145 | 1801295 | 2.71E−07 | −27.30936937 | Hypo-methylated | NEPC\|CRPC-Adeno DMRs |
| chr4 | 1827195 | 1827262 | 0.001802623 | −21.5443609 | Hypo-methylated | NEPC\|CRPC-Adeno DMRs |
| chr4 | 2072559 | 2074840 | 0.000564611 | −22.87341029 | Hypo-methylated | NEPC\|CRPC-Adeno DMRs |
| chr4 | 2298384 | 2298660 | 9.74E−06 | −35.45146199 | Hypo-methylated | NEPC\|CRPC-Adeno DMRs |
| chr4 | 2305552 | 2305706 | 0.000185897 | −28.37493075 | Hypo-methylated | NEPC\|CRPC-Adeno DMRs |
| chr4 | 2305758 | 2306052 | 2.68E−06 | −34.7459964 | Hypo-methylated | NEPC\|CRPC-Adeno DMRs |
| chr4 | 2434539 | 2435008 | 0.006597219 | 23.87213085 | Hyper-methylated | NEPC\|CRPC-Adeno DMRs |
| chr4 | 2451848 | 2452312 | 0.000488426 | −21.58349342 | Hypo-methylated | NEPC\|CRPC-Adeno DMRs |
| chr4 | 2765269 | 2765879 | 4.87E−05 | 30.70148586 | Hyper-methylated | NEPC\|CRPC-Adeno DMRs |
| chr4 | 2925380 | 2926347 | 3.21E−06 | −28.64409357 | Hypo-methylated | NEPC\|CRPC-Adeno DMRs |
| chr4 | 3241235 | 3241827 | 4.24E−06 | −35.8 | Hypo-methylated | NEPC\|CRPC-Adeno DMRs |
| chr4 | 3309966 | 3312002 | 7.41E−07 | −54.48064968 | Hypo-methylated | NEPC\|CRPC-Adeno DMRs |
| chr4 | 3366292 | 3366474 | 0.001694183 | −38.41822612 | Hypo-methylated | NEPC\|CRPC-Adeno DMRs |
| chr4 | 3474193 | 3475085 | 8.06E−06 | −28.33222732 | Hypo-methylated | NEPC\|CRPC-Adeno DMRs |
| chr4 | 3514679 | 3514737 | 0.001694183 | 24.83879142 | Hyper-methylated | NEPC\|CRPC-Adeno DMRs |
| chr4 | 3681675 | 3681807 | 5.17E−06 | −30.86836789 | Hypo-methylated | NEPC\|CRPC-Adeno DMRs |
| chr4 | 3716949 | 3717140 | 0.000303685 | −25.50873753 | Hypo-methylated | NEPC\|CRPC-Adeno DMRs |
| chr4 | 3733981 | 3734137 | 0.001694183 | −34.38421053 | Hypo-methylated | NEPC\|CRPC-Adeno DMRs |
| chr4 | 3752682 | 3752983 | 2.14E−05 | −26.1192147 | Hypo-methylated | NEPC\|CRPC-Adeno DMRs |
| chr4 | 3776475 | 3777021 | 0.000153872 | −33.26239766 | Hypo-methylated | NEPC\|CRPC-Adeno DMRs |
| chr4 | 4358241 | 4361641 | 0.001694183 | −28.7162768 | Hypo-methylated | NEPC\|CRPC-Adeno DMRs |
| chr4 | 6010217 | 6013267 | 0.009458442 | 25.96772262 | Hyper-methylated | NEPC\|CRPC-Adeno DMRs |
| chr4 | 6130336 | 6130384 | 0.009274146 | −45.78013158 | Hypo-methylated | NEPC\|CRPC-Adeno DMRs |
| chr4 | 6339082 | 6344362 | 0.001910709 | −20.0326664 | Hypo-methylated | NEPC\|CRPC-Adeno DMRs |
| chr4 | 6480444 | 6480572 | 0.001802623 | −34.07218045 | Hypo-methylated | NEPC\|CRPC-Adeno DMRs |
| chr4 | 6728813 | 6729056 | 2.68E−06 | −20.47771673 | Hypo-methylated | NEPC\|CRPC-Adeno DMRs |
| chr4 | 6748332 | 6748465 | 0.000549237 | −24.35568333 | Hypo-methylated | NEPC\|CRPC-Adeno DMRs |
| chr4 | 6912457 | 6912580 | 0.001545937 | 20.05412606 | Hyper-methylated | NEPC\|CRPC-Adeno DMRs |
| chr4 | 7124395 | 7126572 | 0.002227988 | −20.14088598 | Hypo-methylated | NEPC\|CRPC-Adeno DMRs |
| chr4 | 7137251 | 7149747 | 0.001798167 | −21.35181579 | Hypo-methylated | NEPC\|CRPC-Adeno DMRs |
| chr4 | 7150037 | 7150269 | 0.007136365 | −28.80891936 | Hypo-methylated | NEPC\|CRPC-Adeno DMRs |
| chr4 | 7279290 | 7279671 | 0.001694183 | −33.24603175 | Hypo-methylated | NEPC\|CRPC-Adeno DMRs |
| chr4 | 7324147 | 7326286 | 0.000224358 | −40.91433007 | Hypo-methylated | NEPC\|CRPC-Adeno DMRs |
| chr4 | 7479768 | 7483229 | 0.000194166 | −23.96654504 | Hypo-methylated | NEPC\|CRPC-Adeno DMRs |
| chr4 | 7647689 | 7648003 | 0.000102266 | −22.97096909 | Hypo-methylated | NEPC\|CRPC-Adeno DMRs |
| chr4 | 7741447 | 7742184 | 4.06E−08 | −20.980728 | Hypo-methylated | NEPC\|CRPC-Adeno DMRs |
| chr4 | 7968382 | 7968597 | 0.000253274 | −27.80275198 | Hypo-methylated | NEPC\|CRPC-Adeno DMRs |
| chr4 | 8012496 | 8012553 | 0.000176134 | −38.73172515 | Hypo-methylated | NEPC\|CRPC-Adeno DMRs |
| chr4 | 8046683 | 8048885 | 0.000291618 | −25.89386272 | Hypo-methylated | NEPC\|CRPC-Adeno DMRs |
| chr4 | 8052250 | 8055954 | 0.000112347 | −22.22377451 | Hypo-methylated | NEPC\|CRPC-Adeno DMRs |
| chr4 | 8137397 | 8137658 | 5.17E−06 | −34.367615 | Hypo-methylated | NEPC\|CRPC-Adeno DMRs |
| chr4 | 8190304 | 8191396 | 9.83E−06 | −34.8466893 | Hypo-methylated | NEPC\|CRPC-Adeno DMRs |
| chr4 | 8272302 | 8272921 | 0.003652661 | −23.68561404 | Hypo-methylated | NEPC\|CRPC-Adeno DMRs |
| chr4 | 8308030 | 8311070 | 4.78E−05 | −24.47625081 | Hypo-methylated | NEPC\|CRPC-Adeno DMRs |
| chr4 | 8502797 | 8519884 | 1.46E−09 | −22.1239748 | Hypo-methylated | NEPC\|CRPC-Adeno DMRs |
| chr4 | 8546717 | 8556387 | 9.53E−06 | −23.64667996 | Hypo-methylated | NEPC\|CRPC-Adeno DMRs |
| chr4 | 8562389 | 8576890 | 9.61E−06 | −26.53628769 | Hypo-methylated | NEPC\|CRPC-Adeno DMRs |
| chr4 | 8859921 | 8859996 | 4.58E−05 | 25.42064777 | Hyper-methylated | NEPC\|CRPC-Adeno DMRs |
| chr4 | 8860131 | 8860181 | 0.000828392 | 27.84342105 | Hyper-methylated | NEPC\|CRPC-Adeno DMRs |
| chr4 | 8862448 | 8863406 | 6.12E−26 | 20.57156307 | Hyper-methylated | NEPC\|CRPC-Adeno DMRs |
| chr4 | 8865001 | 8867237 | 0.0001248 | −21.6385888 | Hypo-methylated | NEPC\|CRPC-Adeno DMRs |
| chr4 | 8873115 | 8874580 | 1.28E−28 | 34.66035175 | Hyper-methylated | NEPC\|CRPC-Adeno DMRs |
| chr4 | 8876016 | 8876216 | 1.89E−09 | 21.82916667 | Hyper-methylated | NEPC\|CRPC-Adeno DMRs |
| chr4 | 8965832 | 8966099 | 0.001594211 | 26.88586944 | Hyper-methylated | NEPC\|CRPC-Adeno DMRs |
| chr4 | 14864563 | 14864658 | 0.000667554 | 23.11719298 | Hyper-methylated | NEPC\|CRPC-Adeno DMRs |
| chr4 | 15779999 | 15780328 | 5.42E−10 | 20.96438845 | Hyper-methylated | NEPC\|CRPC-Adeno DMRs |
| chr4 | 15907622 | 15907745 | 3.76E−08 | −29.14906256 | Hypo-methylated | NEPC\|CRPC-Adeno DMRs |
| chr4 | 21950297 | 21950513 | 5.04E−12 | 27.10330817 | Hyper-methylated | NEPC\|CRPC-Adeno DMRs |
| chr4 | 22004221 | 22261118 | 0.00047236 | −25.07491056 | Hypo-methylated | NEPC\|CRPC-Adeno DMRs |
| chr4 | 22601034 | 22605890 | 9.54E−05 | −20.18913847 | Hypo-methylated | NEPC\|CRPC-Adeno DMRs |
| chr4 | 25972024 | 25972308 | 0.006785531 | −29.5401133 | Hypo-methylated | NEPC\|CRPC-Adeno DMRs |
| chr4 | 31999120 | 32143213 | 0.004906972 | −21.40697095 | Hypo-methylated | NEPC\|CRPC-Adeno DMRs |
| chr4 | 35465234 | 35496781 | 0.003327791 | −23.69766082 | Hypo-methylated | NEPC\|CRPC-Adeno DMRs |
| chr4 | 37245332 | 37247113 | 2.12E−32 | 27.71839273 | Hyper-methylated | NEPC\|CRPC-Adeno DMRs |
| chr4 | 37891793 | 37892014 | 8.67E−07 | −28.78396954 | Hypo-methylated | NEPC\|CRPC-Adeno DMRs |
| chr4 | 44449486 | 44450642 | 2.59E−31 | 39.84740685 | Hyper-methylated | NEPC\|CRPC-Adeno DMRs |
| chr4 | 46391929 | 46392389 | 3.90E−10 | 30.72768404 | Hyper-methylated | NEPC\|CRPC-Adeno DMRs |
| chr4 | 48995814 | 49028259 | 0.00064342 | −25.52280702 | Hypo-methylated | NEPC\|CRPC-Adeno DMRs |
| chr4 | 52943055 | 52943231 | 1.37E−07 | 22.2200188 | Hyper-methylated | NEPC\|CRPC-Adeno DMRs |
| chr4 | 69264478 | 69265296 | 0.000549237 | −22.9556391 | Hypo-methylated | NEPC\|CRPC-Adeno DMRs |
| chr4 | 73573436 | 73617031 | 0.005557404 | −23.02609357 | Hypo-methylated | NEPC\|CRPC-Adeno DMRs |
| chr4 | 75860240 | 75860322 | 0.000488426 | −29.80107212 | Hypo-methylated | NEPC\|CRPC-Adeno DMRs |
| chr4 | 77342147 | 77342691 | 1.24E−09 | −23.00629699 | Hypo-methylated | NEPC\|CRPC-Adeno DMRs |
| chr4 | 100573828 | 100574479 | 5.11E−12 | −27.28129275 | Hypo-methylated | NEPC\|CRPC-Adeno DMRs |
| chr4 | 103131764 | 103158583 | 0.005737741 | −23.25396825 | Hypo-methylated | NEPC\|CRPC-Adeno DMRs |
| chr4 | 109092629 | 109092749 | 0.000263232 | 26.56473684 | Hyper-methylated | NEPC\|CRPC-Adeno DMRs |
| chr4 | 109692948 | 109709129 | 0.0001635 | −27.47655502 | Hypo-methylated | NEPC\|CRPC-Adeno DMRs |
| chr4 | 111178753 | 111367020 | 3.47E−08 | −31.98680845 | Hypo-methylated | NEPC\|CRPC-Adeno DMRs |
| chr4 | 111543324 | 111543521 | 0.001345794 | 20.90681244 | Hyper-methylated | NEPC\|CRPC-Adeno DMRs |
| chr4 | 111544325 | 111544400 | 4.65E−07 | 20.64331984 | Hyper-methylated | NEPC\|CRPC-Adeno DMRs |
| chr4 | 111555162 | 111555247 | 0.000118331 | 24.78863826 | Hyper-methylated | NEPC\|CRPC-Adeno DMRs |
| chr4 | 113430635 | 113431994 | 1.28E−09 | −20.32926857 | Hypo-methylated | NEPC\|CRPC-Adeno DMRs |
| chr4 | 113442037 | 113444633 | 0.000670739 | 20.5255289 | Hyper-methylated | NEPC\|CRPC-Adeno DMRs |
| chr4 | 119186690 | 119186881 | 1.00E−11 | 37.90779133 | Hyper-methylated | NEPC\|CRPC-Adeno DMRs |
| chr4 | 122080124 | 122299730 | 2.12E−07 | −22.68246801 | Hypo-methylated | NEPC\|CRPC-Adeno DMRs |
| chr4 | 122685757 | 122686454 | 9.67E−14 | 29.48130205 | Hyper-methylated | NEPC\|CRPC-Adeno DMRs |
| chr4 | 134071988 | 134073990 | 3.79E−27 | 20.93377544 | Hyper-methylated | NEPC\|CRPC-Adeno DMRs |
| chr4 | 139726023 | 139726078 | 0.001694183 | 25.81993177 | Hyper-methylated | NEPC\|CRPC-Adeno DMRs |
| chr4 | 140707856 | 140760460 | 0.005737741 | −34.00315546 | Hypo-methylated | NEPC\|CRPC-Adeno DMRs |
| chr4 | 143766812 | 143767085 | 5.97E−11 | −21.33005729 | Hypo-methylated | NEPC\|CRPC-Adeno DMRs |
| chr4 | 144833165 | 144833504 | 7.16E−11 | 25.91972915 | Hyper-methylated | NEPC\|CRPC-Adeno DMRs |
| chr4 | 147559923 | 147559956 | 0.001694183 | −21.32539683 | Hypo-methylated | NEPC\|CRPC-Adeno DMRs |
| chr4 | 147561282 | 147562108 | 0.000100877 | 21.86546956 | Hyper-methylated | NEPC\|CRPC-Adeno DMRs |
| chr4 | 147569270 | 147569291 | 0.001694183 | 21.37329435 | Hyper-methylated | NEPC\|CRPC-Adeno DMRs |
| chr4 | 147901913 | 148151807 | 0.00025311 | −23.67947345 | Hypo-methylated | NEPC\|CRPC-Adeno DMRs |
| chr4 | 148213356 | 148268629 | 1.70E−05 | −29.28543172 | Hypo-methylated | NEPC\|CRPC-Adeno DMRs |
| chr4 | 153941239 | 153941390 | 0.004750844 | −30.23157895 | Hypo-methylated | NEPC\|CRPC-Adeno DMRs |
| chr4 | 154143655 | 154144921 | 4.12E−16 | −27.29381106 | Hypo-methylated | NEPC\|CRPC-Adeno DMRs |
| chr4 | 154680909 | 154681339 | 9.88E−11 | 23.42486388 | Hyper-methylated | NEPC\|CRPC-Adeno DMRs |
| chr4 | 154709607 | 154709685 | 0.000549237 | 25.83609023 | Hyper-methylated | NEPC\|CRPC-Adeno DMRs |
| chr4 | 154709813 | 154710192 | 1.10E−10 | 28.27913385 | Hyper-methylated | NEPC\|CRPC-Adeno DMRs |
| chr4 | 154711171 | 154711347 | 0.001694183 | −25.4120614 | Hypo-methylated | NEPC\|CRPC-Adeno DMRs |
| chr4 | 154712129 | 154713601 | 3.29E−12 | 25.36497873 | Hyper-methylated | NEPC\|CRPC-Adeno DMRs |
| chr4 | 155411495 | 155411720 | 1.31E−14 | 27.98769186 | Hyper-methylated | NEPC\|CRPC-Adeno DMRs |
| chr4 | 156135533 | 156253885 | 0.004750844 | −37.45413534 | Hypo-methylated | NEPC\|CRPC-Adeno DMRs |
| chr4 | 158141449 | 158143729 | 5.05E−24 | 20.03226006 | Hyper-methylated | NEPC\|CRPC-Adeno DMRs |
| chr4 | 160393088 | 160595703 | 7.96E−06 | −25.7773823 | Hypo-methylated | NEPC\|CRPC-Adeno DMRs |
| chr4 | 160652638 | 160790501 | 1.86E−06 | −26.32935372 | Hypo-methylated | NEPC\|CRPC-Adeno DMRs |
| chr4 | 160917553 | 161230823 | 4.74E−05 | −20.76706679 | Hypo-methylated | NEPC\|CRPC-Adeno DMRs |
| chr4 | 164253010 | 164253056 | 0.000549237 | 26.1337146 | Hyper-methylated | NEPC\|CRPC-Adeno DMRs |
| chr4 | 165703722 | 165854644 | 1.14E−07 | −22.79351436 | Hypo-methylated | NEPC\|CRPC-Adeno DMRs |
| chr4 | 165952764 | 165952870 | 2.91E−05 | −21.80339932 | Hypo-methylated | NEPC\|CRPC-Adeno DMRs |
| chr4 | 165953157 | 165976637 | 0.000711935 | −24.31388889 | Hypo-methylated | NEPC\|CRPC-Adeno DMRs |
| chr4 | 166500186 | 166720741 | 0.000112177 | −24.22509659 | Hypo-methylated | NEPC\|CRPC-Adeno DMRs |
| chr4 | 170785691 | 170893524 | 8.77E−06 | −26.8739956 | Hypo-methylated | NEPC\|CRPC-Adeno DMRs |
| chr4 | 174437845 | 174437944 | 0.002927544 | 22.5251462 | Hyper-methylated | NEPC\|CRPC-Adeno DMRs |
| chr4 | 174450465 | 174451873 | 6.56E−16 | 22.5629451 | Hyper-methylated | NEPC\|CRPC-Adeno DMRs |
| chr4 | 174459376 | 174459588 | 0.000667554 | 32.42068161 | Hyper-methylated | NEPC\|CRPC-Adeno DMRs |
| chr4 | 175339069 | 175433093 | 0.000263673 | −26.26754386 | Hypo-methylated | NEPC\|CRPC-Adeno DMRs |
| chr4 | 178782781 | 178832448 | 0.001802623 | −23.4431391 | Hypo-methylated | NEPC\|CRPC-Adeno DMRs |
| chr4 | 183081214 | 183120339 | 1.27E−06 | −30.30664201 | Hypo-methylated | NEPC\|CRPC-Adeno DMRs |
| chr4 | 183241123 | 183311133 | 0.00010588 | −25.35918518 | Hypo-methylated | NEPC\|CRPC-Adeno DMRs |
| chr4 | 183402714 | 183493254 | 2.92E−09 | −32.79803653 | Hypo-methylated | NEPC\|CRPC-Adeno DMRs |
| chr4 | 183529792 | 183698872 | 1.20E−14 | −29.65124633 | Hypo-methylated | NEPC\|CRPC-Adeno DMRs |
| chr4 | 183801572 | 183801949 | 0.001694183 | −23.68157895 | Hypo-methylated | NEPC\|CRPC-Adeno DMRs |
| chr4 | 186545426 | 186545487 | 0.000176134 | −30.88093933 | Hypo-methylated | NEPC\|CRPC-Adeno DMRs |
| chr4 | 187025976 | 187028029 | 6.02E−19 | −21.34827446 | Hypo-methylated | NEPC\|CRPC-Adeno DMRs |
| chr4 | 187225016 | 187246813 | 0.005899637 | −25.28902649 | Hypo-methylated | NEPC\|CRPC-Adeno DMRs |
| chr4 | 187580851 | 187581221 | 0.000184724 | −24.95337308 | Hypo-methylated | NEPC\|CRPC-Adeno DMRs |
| chr4 | 188377454 | 188377608 | 0.000549237 | −34.25639098 | Hypo-methylated | NEPC\|CRPC-Adeno DMRs |
| chr4 | 188964162 | 188964332 | 0.002976353 | −27.25124717 | Hypo-methylated | NEPC\|CRPC-Adeno DMRs |
| chr4 | 189084649 | 189100194 | 1.27E−05 | −21.01399086 | Hypo-methylated | NEPC\|CRPC-Adeno DMRs |
| chr4 | 189155003 | 189167421 | 0.002569607 | −24.64791701 | Hypo-methylated | NEPC\|CRPC-Adeno DMRs |
| chr4 | 189220569 | 189224791 | 5.36E−06 | −26.43492934 | Hypo-methylated | NEPC\|CRPC-Adeno DMRs |
| chr5 | 491048 | 491463 | 2.66E−08 | −28.92773025 | Hypo-methylated | NEPC\|CRPC-Adeno DMRs |
| chr5 | 532829 | 533068 | 0.005899637 | −24.31432749 | Hypo-methylated | NEPC\|CRPC-Adeno DMRs |
| chr5 | 607867 | 607969 | 0.001694183 | −28.22245614 | Hypo-methylated | NEPC\|CRPC-Adeno DMRs |
| chr5 | 628539 | 629117 | 0.006785531 | −24.25783884 | Hypo-methylated | NEPC\|CRPC-Adeno DMRs |
| chr5 | 669261 | 669386 | 0.001197874 | 30.94987843 | Hyper-methylated | NEPC\|CRPC-Adeno DMRs |
| chr5 | 961668 | 961903 | 1.55E−06 | −23.43138688 | Hypo-methylated | NEPC\|CRPC-Adeno DMRs |
| chr5 | 993246 | 1004433 | 1.98E−10 | −40.44307174 | Hypo-methylated | NEPC\|CRPC-Adeno DMRs |
| chr5 | 1125206 | 1130375 | 0.002094266 | −25.08654971 | Hypo-methylated | NEPC\|CRPC-Adeno DMRs |
| chr5 | 1193728 | 1194028 | 2.64E−06 | −23.43138402 | Hypo-methylated | NEPC\|CRPC-Adeno DMRs |
| chr5 | 1394522 | 1394673 | 0.006847068 | −27.14201681 | Hypo-methylated | NEPC\|CRPC-Adeno DMRs |
| chr5 | 1400370 | 1401940 | 0.001545937 | −23.71578947 | Hypo-methylated | NEPC\|CRPC-Adeno DMRs |
| chr5 | 1444727 | 1444779 | 0.000224358 | 22.92894737 | Hyper-methylated | NEPC\|CRPC-Adeno DMRs |
| chr5 | 1513080 | 1513260 | 0.001694183 | −25.01560847 | Hypo-methylated | NEPC\|CRPC-Adeno DMRs |
| chr5 | 1643928 | 1644137 | 0.001065261 | 23.71476608 | Hyper-methylated | NEPC\|CRPC-Adeno DMRs |
| chr5 | 1686216 | 1701618 | 9.09E−05 | −27.965722 | Hypo-methylated | NEPC\|CRPC-Adeno DMRs |
| chr5 | 1704542 | 1725347 | 4.71E−07 | −23.24799112 | Hypo-methylated | NEPC\|CRPC-Adeno DMRs |
| chr5 | 1751468 | 1762953 | 0.000450645 | −25.04540898 | Hypo-methylated | NEPC\|CRPC-Adeno DMRs |
| chr5 | 1777897 | 1787184 | 0.000220525 | −20.11717605 | Hypo-methylated | NEPC\|CRPC-Adeno DMRs |
| chr5 | 1787937 | 1789080 | 0.003525992 | −43.53194014 | Hypo-methylated | NEPC\|CRPC-Adeno DMRs |
| chr5 | 1835051 | 1836678 | 0.000642419 | −25.0901864 | Hypo-methylated | NEPC\|CRPC-Adeno DMRs |
| chr5 | 1837723 | 1840348 | 0.000579916 | −20.45676692 | Hypo-methylated | NEPC\|CRPC-Adeno DMRs |
| chr5 | 1850715 | 1851120 | 0.002094266 | −23.03810507 | Hypo-methylated | NEPC\|CRPC-Adeno DMRs |
| chr5 | 1858706 | 1869906 | 1.47E−09 | −29.91434874 | Hypo-methylated | NEPC\|CRPC-Adeno DMRs |
| chr5 | 2213580 | 2261535 | 5.83E−10 | −25.43094034 | Hypo-methylated | NEPC\|CRPC-Adeno DMRs |
| chr5 | 2301017 | 2334910 | 2.87E−05 | −28.18859284 | Hypo-methylated | NEPC\|CRPC-Adeno DMRs |
| chr5 | 2671287 | 2697609 | 4.11E−07 | −33.12029584 | Hypo-methylated | NEPC\|CRPC-Adeno DMRs |
| chr5 | 3536326 | 3538047 | 0.000231593 | −27.932665 | Hypo-methylated | NEPC\|CRPC-Adeno DMRs |
| chr5 | 3594514 | 3594587 | 0.00222033 | 22.14407895 | Hyper-methylated | NEPC\|CRPC-Adeno DMRs |
| chr5 | 3595765 | 3596010 | 2.02E−06 | 21.64462719 | Hyper-methylated | NEPC\|CRPC-Adeno DMRs |
| chr5 | 3596307 | 3596795 | 1.16E−14 | 23.44162339 | Hyper-methylated | NEPC\|CRPC-Adeno DMRs |
| chr5 | 3600783 | 3603155 | 8.35E−15 | 22.84933937 | Hyper-methylated | NEPC\|CRPC-Adeno DMRs |
| chr5 | 5231182 | 5317862 | 1.03E−06 | −24.45755393 | Hypo-methylated | NEPC\|CRPC-Adeno DMRs |
| chr5 | 5337138 | 5421549 | 7.94E−07 | −26.21022812 | Hypo-methylated | NEPC\|CRPC-Adeno DMRs |
| chr5 | 6181706 | 6191114 | 0.002859601 | −24.1171345 | Hypo-methylated | NEPC\|CRPC-Adeno DMRs |
| chr5 | 6609478 | 6614935 | 0.000549237 | 26.78796992 | Hyper-methylated | NEPC\|CRPC-Adeno DMRs |
| chr5 | 7077307 | 7077546 | 0.00041315 | −25.21176088 | Hypo-methylated | NEPC\|CRPC-Adeno DMRs |
| chr5 | 8430191 | 8789256 | 1.62E−16 | 33.12498171 | Hyper-methylated | NEPC\|CRPC-Adeno DMRs |
| chr5 | 10860497 | 11313834 | 2.44E−17 | −28.0495594 | Hypo-methylated | NEPC\|CRPC-Adeno DMRs |
| chr5 | 11397188 | 11857366 | 3.40E−10 | −33.659854 | Hypo-methylated | NEPC\|CRPC-Adeno DMRs |
| chr5 | 16867031 | 16867167 | 0.003525992 | −46.08001949 | Hypo-methylated | NEPC\|CRPC-Adeno DMRs |
| chr5 | 19142240 | 19143195 | 0.000732983 | 21.70137275 | Hyper-methylated | NEPC\|CRPC-Adeno DMRs |
| chr5 | 22654559 | 22972651 | 0.00011018 | −28.1783577 | Hypo-methylated | NEPC\|CRPC-Adeno DMRs |
| chr5 | 23688436 | 23688549 | 0.009458442 | −27.97894737 | Hypo-methylated | NEPC\|CRPC-Adeno DMRs |
| chr5 | 29390802 | 29390973 | 5.50E−05 | −25.14139051 | Hypo-methylated | NEPC\|CRPC-Adeno DMRs |
| chr5 | 31589171 | 31610599 | 0.001802623 | −20.12206356 | Hypo-methylated | NEPC\|CRPC-Adeno DMRs |
| chr5 | 31692445 | 31693027 | 0.007136365 | 28.55391657 | Hyper-methylated | NEPC\|CRPC-Adeno DMRs |
| chr5 | 31693538 | 31727938 | 2.91E−05 | −21.46961097 | Hypo-methylated | NEPC\|CRPC-Adeno DMRs |
| chr5 | 32251322 | 32264335 | 0.006785531 | 20.54736842 | Hyper-methylated | NEPC\|CRPC-Adeno DMRs |
| chr5 | 32721605 | 33311293 | 2.10E−07 | −33.78018252 | Hypo-methylated | NEPC\|CRPC-Adeno DMRs |
| chr5 | 33937050 | 33938305 | 9.51E−10 | 24.61330833 | Hyper-methylated | NEPC\|CRPC-Adeno DMRs |
| chr5 | 35091209 | 35134654 | 0.001076438 | −38.62090322 | Hypo-methylated | NEPC\|CRPC-Adeno DMRs |
| chr5 | 35619506 | 35892413 | 7.81E−09 | −35.5425661 | Hypo-methylated | NEPC\|CRPC-Adeno DMRs |
| chr5 | 36727808 | 36737861 | 0.000324528 | −26.73156981 | Hypo-methylated | NEPC\|CRPC-Adeno DMRs |
| chr5 | 37666116 | 37666267 | 5.50E−05 | −27.19408707 | Hypo-methylated | NEPC\|CRPC-Adeno DMRs |
| chr5 | 37862565 | 37952562 | 0.000200729 | −28.0684014 | Hypo-methylated | NEPC\|CRPC-Adeno DMRs |
| chr5 | 38113779 | 38190504 | 2.14E−06 | −26.21596147 | Hypo-methylated | NEPC\|CRPC-Adeno DMRs |
| chr5 | 38211357 | 38244733 | 0.007832634 | −21.04698204 | Hypo-methylated | NEPC\|CRPC-Adeno DMRs |
| chr5 | 38290998 | 38355161 | 5.10E−05 | −25.96060855 | Hypo-methylated | NEPC\|CRPC-Adeno DMRs |
| chr5 | 39132256 | 39333984 | 5.29E−07 | −25.60535782 | Hypo-methylated | NEPC\|CRPC-Adeno DMRs |
| chr5 | 39343335 | 39390614 | 5.75E−05 | −36.85578947 | Hypo-methylated | NEPC\|CRPC-Adeno DMRs |
| chr5 | 39429528 | 39622086 | 2.41E−06 | −34.54174325 | Hypo-methylated | NEPC\|CRPC-Adeno DMRs |
| chr5 | 39649723 | 40539431 | 8.04E−18 | −28.16500715 | Hypo-methylated | NEPC\|CRPC-Adeno DMRs |
| chr5 | 42516504 | 42752667 | 5.50E−05 | −31.89449604 | Hypo-methylated | NEPC\|CRPC-Adeno DMRs |
| chr5 | 43000398 | 43000777 | 9.48E−10 | −22.56860624 | Hypo-methylated | NEPC\|CRPC-Adeno DMRs |
| chr5 | 45695618 | 45696419 | 7.45E−20 | 29.32638602 | Hyper-methylated | NEPC\|CRPC-Adeno DMRs |
| chr5 | 49897942 | 49903511 | 0.000549237 | −20.92837302 | Hypo-methylated | NEPC\|CRPC-Adeno DMRs |
| chr5 | 50685513 | 50695269 | 4.32E−20 | 23.85970528 | Hyper-methylated | NEPC\|CRPC-Adeno DMRs |
| chr5 | 53948180 | 53988748 | 0.006785531 | −22.22867647 | Hypo-methylated | NEPC\|CRPC-Adeno DMRs |
| chr5 | 54179645 | 54180111 | 4.20E−09 | 27.60839089 | Hyper-methylated | NEPC\|CRPC-Adeno DMRs |
| chr5 | 56897339 | 57058095 | 1.12E−05 | −23.87566471 | Hypo-methylated | NEPC\|CRPC-Adeno DMRs |
| chr5 | 62324096 | 62850021 | 0.001026234 | −21.78368514 | Hypo-methylated | NEPC\|CRPC-Adeno DMRs |
| chr5 | 67483406 | 67483572 | 1.50E−06 | −32.34401604 | Hypo-methylated | NEPC\|CRPC-Adeno DMRs |
| chr5 | 72746667 | 72746714 | 0.001694183 | 27.68421053 | Hyper-methylated | NEPC\|CRPC-Adeno DMRs |
| chr5 | 76022280 | 76028649 | 0.001552631 | −22.64898325 | Hypo-methylated | NEPC\|CRPC-Adeno DMRs |
| chr5 | 76061717 | 76078695 | 0.001545937 | −21.58327914 | Hypo-methylated | NEPC\|CRPC-Adeno DMRs |
| chr5 | 76249807 | 76250089 | 3.01E−08 | 21.51725325 | Hyper-methylated | NEPC\|CRPC-Adeno DMRs |
| chr5 | 76932335 | 76932495 | 8.24E−05 | 22.06547128 | Hyper-methylated | NEPC\|CRPC-Adeno DMRs |
| chr5 | 77815597 | 77889025 | 0.001452666 | −23.08098595 | Hypo-methylated | NEPC\|CRPC-Adeno DMRs |
| chr5 | 79469275 | 79485204 | 0.000324528 | 24.55337762 | Hyper-methylated | NEPC\|CRPC-Adeno DMRs |
| chr5 | 79865118 | 79865343 | 5.01E−05 | −22.12807018 | Hypo-methylated | NEPC\|CRPC-Adeno DMRs |
| chr5 | 83679871 | 83680735 | 1.17E−12 | 22.62946109 | Hyper-methylated | NEPC\|CRPC-Adeno DMRs |
| chr5 | 86765324 | 86795831 | 6.00E−07 | −37.60657035 | Hypo-methylated | NEPC\|CRPC-Adeno DMRs |
| chr5 | 86883367 | 87066719 | 0.006785531 | −22.46150145 | Hypo-methylated | NEPC\|CRPC-Adeno DMRs |
| chr5 | 87968636 | 87971646 | 6.35E−13 | 26.16172775 | Hyper-methylated | NEPC\|CRPC-Adeno DMRs |
| chr5 | 87974174 | 87976063 | 0.000220189 | 21.77493192 | Hyper-methylated | NEPC\|CRPC-Adeno DMRs |
| chr5 | 89006887 | 89704529 | 2.98E−05 | −21.42766445 | Hypo-methylated | NEPC\|CRPC-Adeno DMRs |
| chr5 | 95767768 | 95767864 | 0.003525992 | −24.34814815 | Hypo-methylated | NEPC\|CRPC-Adeno DMRs |
| chr5 | 96038303 | 96038858 | 3.02E−14 | 28.67572751 | Hyper-methylated | NEPC\|CRPC-Adeno DMRs |
| chr5 | 96915078 | 97074935 | 0.002094266 | −24.71471544 | Hypo-methylated | NEPC\|CRPC-Adeno DMRs |
| chr5 | 113259183 | 113259266 | 0.001694183 | −31.04210526 | Hypo-methylated | NEPC\|CRPC-Adeno DMRs |
| chr5 | 113498744 | 113658960 | 1.41E−05 | −30.62287889 | Hypo-methylated | NEPC\|CRPC-Adeno DMRs |
| chr5 | 113721736 | 114031471 | 4.65E−07 | −31.84509109 | Hypo-methylated | NEPC\|CRPC-Adeno DMRs |
| chr5 | 114054410 | 114265033 | 1.92E−06 | −23.85403307 | Hypo-methylated | NEPC\|CRPC-Adeno DMRs |
| chr5 | 115297294 | 115298092 | 2.70E−07 | 23.95747703 | Hyper-methylated | NEPC\|CRPC-Adeno DMRs |
| chr5 | 115298702 | 115298783 | 5.50E−05 | 21.64619883 | Hyper-methylated | NEPC\|CRPC-Adeno DMRs |
| chr5 | 119008929 | 119162031 | 1.34E−07 | −21.04597621 | Hypo-methylated | NEPC\|CRPC-Adeno DMRs |
| chr5 | 120154181 | 120245345 | 0.001549499 | −22.87368421 | Hypo-methylated | NEPC\|CRPC-Adeno DMRs |
| chr5 | 122422309 | 122423615 | 4.23E−09 | 30.39827558 | Hyper-methylated | NEPC\|CRPC-Adeno DMRs |
| chr5 | 125784436 | 125801624 | 0.000200845 | −20.13070175 | Hypo-methylated | NEPC\|CRPC-Adeno DMRs |
| chr5 | 126626530 | 126626849 | 0.000424361 | 26.47919799 | Hyper-methylated | NEPC\|CRPC-Adeno DMRs |
| chr5 | 127929967 | 128268295 | 1.85E−05 | −26.69549016 | Hypo-methylated | NEPC\|CRPC-Adeno DMRs |
| chr5 | 128920788 | 129000785 | 8.98E−08 | −40.22752932 | Hypo-methylated | NEPC\|CRPC-Adeno DMRs |
| chr5 | 129242259 | 129813660 | 0.002215223 | −23.6386056 | Hypo-methylated | NEPC\|CRPC-Adeno DMRs |
| chr5 | 129899475 | 130101707 | 0.000209051 | −25.75539283 | Hypo-methylated | NEPC\|CRPC-Adeno DMRs |
| chr5 | 130281834 | 130331046 | 4.98E−06 | −30.01544394 | Hypo-methylated | NEPC\|CRPC-Adeno DMRs |
| chr5 | 132167655 | 132173260 | 7.67E−05 | 21.36149143 | Hyper-methylated | NEPC\|CRPC-Adeno DMRs |
| chr5 | 132707535 | 132707620 | 0.001694183 | −43.18323587 | Hypo-methylated | NEPC\|CRPC-Adeno DMRs |
| chr5 | 134477662 | 134515249 | 0.00217795 | −32.9503096 | Hypo-methylated | NEPC\|CRPC-Adeno DMRs |
| chr5 | 134582848 | 134583164 | 0.00020815 | −27.9623968 | Hypo-methylated | NEPC\|CRPC-Adeno DMRs |
| chr5 | 138281818 | 138284832 | 0.005297267 | −24.62317559 | Hypo-methylated | NEPC\|CRPC-Adeno DMRs |
| chr5 | 138441349 | 138441563 | 0.000176134 | −72.83048783 | Hypo-methylated | NEPC\|CRPC-Adeno DMRs |
| chr5 | 140011903 | 140012735 | 3.67E−07 | −25.94475993 | Hypo-methylated | NEPC\|CRPC-Adeno DMRs |
| chr5 | 140536613 | 140626811 | 1.72E−31 | 23.92451239 | Hyper-methylated | NEPC\|CRPC-Adeno DMRs |
| chr5 | 140710358 | 140718835 | 7.26E−05 | 21.82257932 | Hyper-methylated | NEPC\|CRPC-Adeno DMRs |
| chr5 | 140731220 | 140746961 | 1.47E−15 | 25.84078042 | Hyper-methylated | NEPC\|CRPC-Adeno DMRs |
| chr5 | 141087185 | 141133473 | 6.07E−05 | 23.08007373 | Hyper-methylated | NEPC\|CRPC-Adeno DMRs |
| chr5 | 141263002 | 141263271 | 1.36E−13 | 27.20863158 | Hyper-methylated | NEPC\|CRPC-Adeno DMRs |
| chr5 | 141566562 | 141600717 | 0.006494419 | 24.1505214 | Hyper-methylated | NEPC\|CRPC-Adeno DMRs |
| chr5 | 144867410 | 144867547 | 0.001694183 | −23.73368665 | Hypo-methylated | NEPC\|CRPC-Adeno DMRs |
| chr5 | 145725544 | 145725821 | 1.57E−06 | 23.39155818 | Hyper-methylated | NEPC\|CRPC-Adeno DMRs |
| chr5 | 149570212 | 149572479 | 2.91E−05 | −30.75812191 | Hypo-methylated | NEPC\|CRPC-Adeno DMRs |
| chr5 | 149979489 | 149980177 | 0.006890179 | −25.38671679 | Hypo-methylated | NEPC\|CRPC-Adeno DMRs |
| chr5 | 150614450 | 150614558 | 0.003475856 | 24.35338346 | Hyper-methylated | NEPC\|CRPC-Adeno DMRs |
| chr5 | 153853305 | 153855316 | 3.45E−07 | 25.49400403 | Hyper-methylated | NEPC\|CRPC-Adeno DMRs |
| chr5 | 153858648 | 153862193 | 6.94E−21 | 28.25829046 | Hyper-methylated | NEPC\|CRPC-Adeno DMRs |
| chr5 | 153896878 | 153901173 | 0.000176134 | 20.55437204 | Hyper-methylated | NEPC\|CRPC-Adeno DMRs |
| chr5 | 157098822 | 157098921 | 0.000100851 | 25.96363636 | Hyper-methylated | NEPC\|CRPC-Adeno DMRs |
| chr5 | 158335905 | 158342242 | 0.001694183 | −34.98245614 | Hypo-methylated | NEPC\|CRPC-Adeno DMRs |
| chr5 | 159964850 | 160082980 | 0.000921835 | −21.92822967 | Hypo-methylated | NEPC\|CRPC-Adeno DMRs |
| chr5 | 160592487 | 160593463 | 2.78E−10 | −34.87710834 | Hypo-methylated | NEPC\|CRPC-Adeno DMRs |
| chr5 | 161397997 | 161489472 | 0.002094266 | −28.33618421 | Hypo-methylated | NEPC\|CRPC-Adeno DMRs |
| chr5 | 165965200 | 165983547 | 0.003113626 | −25.15536062 | Hypo-methylated | NEPC\|CRPC-Adeno DMRs |
| chr5 | 166211800 | 166211938 | 1.77E−05 | −32.10517962 | Hypo-methylated | NEPC\|CRPC-Adeno DMRs |
| chr5 | 166216555 | 166358616 | 4.65E−10 | −21.25623053 | Hypo-methylated | NEPC\|CRPC-Adeno DMRs |
| chr5 | 168031856 | 168070922 | 0.008985252 | −20.85302144 | Hypo-methylated | NEPC\|CRPC-Adeno DMRs |
| chr5 | 169780009 | 169814440 | 0.000114388 | −21.95998254 | Hypo-methylated | NEPC\|CRPC-Adeno DMRs |
| chr5 | 169905415 | 169916149 | 0.000277187 | −30.32534909 | Hypo-methylated | NEPC\|CRPC-Adeno DMRs |
| chr5 | 170068701 | 170094209 | 9.28E−06 | −23.78628472 | Hypo-methylated | NEPC\|CRPC-Adeno DMRs |
| chr5 | 170171206 | 170171278 | 2.91E−05 | 20.41866029 | Hyper-methylated | NEPC\|CRPC-Adeno DMRs |
| chr5 | 172281596 | 172285250 | 0.008985252 | 36.9627193 | Hyper-methylated | NEPC\|CRPC-Adeno DMRs |
| chr5 | 172640852 | 172655078 | 0.00324027 | −26.57418516 | Hypo-methylated | NEPC\|CRPC-Adeno DMRs |
| chr5 | 172717583 | 172718973 | 0.001156603 | −39.31137218 | Hypo-methylated | NEPC\|CRPC-Adeno DMRs |
| chr5 | 172751331 | 172751409 | 0.000549237 | −35.98646617 | Hypo-methylated | NEPC\|CRPC-Adeno DMRs |
| chr5 | 172924855 | 172924910 | 0.003525992 | 23.18267544 | Hyper-methylated | NEPC\|CRPC-Adeno DMRs |
| chr5 | 172982435 | 172982612 | 1.77E−05 | −21.40207337 | Hypo-methylated | NEPC\|CRPC-Adeno DMRs |
| chr5 | 173060626 | 173060693 | 0.001694183 | −50.75175439 | Hypo-methylated | NEPC\|CRPC-Adeno DMRs |
| chr5 | 173416735 | 173416924 | 0.000324528 | −32.68230295 | Hypo-methylated | NEPC\|CRPC-Adeno DMRs |
| chr5 | 173937899 | 173954821 | 0.007379618 | −27.30172306 | Hypo-methylated | NEPC\|CRPC-Adeno DMRs |
| chr5 | 175222595 | 175222664 | 0.001694183 | 20.47368421 | Hyper-methylated | NEPC\|CRPC-Adeno DMRs |
| chr5 | 175299618 | 175305219 | 7.59E−05 | −20.60035425 | Hypo-methylated | NEPC\|CRPC-Adeno DMRs |
| chr5 | 175488179 | 175488850 | 7.42E−07 | 31.99841923 | Hyper-methylated | NEPC\|CRPC-Adeno DMRs |
| chr5 | 176023532 | 176024630 | 1.66E−09 | 32.75785602 | Hyper-methylated | NEPC\|CRPC-Adeno DMRs |
| chr5 | 176225635 | 176226125 | 1.37E−07 | −29.15918964 | Hypo-methylated | NEPC\|CRPC-Adeno DMRs |
| chr5 | 176799200 | 176803611 | 0.001549499 | 24.3224681 | Hyper-methylated | NEPC\|CRPC-Adeno DMRs |
| chr5 | 177513281 | 177513608 | 3.60E−08 | −26.53032288 | Hypo-methylated | NEPC\|CRPC-Adeno DMRs |
| chr5 | 177694310 | 177729158 | 1.16E−17 | −22.86636965 | Hypo-methylated | NEPC\|CRPC-Adeno DMRs |
| chr5 | 177804400 | 177808731 | 3.56E−06 | −33.93883514 | Hypo-methylated | NEPC\|CRPC-Adeno DMRs |
| chr5 | 177831014 | 177849724 | 1.92E−09 | −22.83100111 | Hypo-methylated | NEPC\|CRPC-Adeno DMRs |
| chr5 | 177852613 | 177852714 | 0.000892405 | −22.98275272 | Hypo-methylated | NEPC\|CRPC-Adeno DMRs |
| chr5 | 177936432 | 177964582 | 4.91E−11 | −22.72903964 | Hypo-methylated | NEPC\|CRPC-Adeno DMRs |
| chr5 | 178368052 | 178368441 | 9.56E−09 | 27.05484081 | Hyper-methylated | NEPC\|CRPC-Adeno DMRs |
| chr5 | 178415954 | 178416070 | 0.007136365 | −34.0329753 | Hypo-methylated | NEPC\|CRPC-Adeno DMRs |
| chr5 | 178421442 | 178422158 | 2.80E−34 | 37.76057537 | Hyper-methylated | NEPC\|CRPC-Adeno DMRs |
| chr5 | 179051472 | 179051611 | 3.05E−05 | −25.67095517 | Hypo-methylated | NEPC\|CRPC-Adeno DMRs |
| chr5 | 179401758 | 179401883 | 0.000100851 | −59.28431685 | Hypo-methylated | NEPC\|CRPC-Adeno DMRs |
| chr5 | 179546299 | 179553332 | 0.002976353 | −22.9599832 | Hypo-methylated | NEPC\|CRPC-Adeno DMRs |
| chr5 | 179555790 | 179563506 | 0.000176134 | −23.24616634 | Hypo-methylated | NEPC\|CRPC-Adeno DMRs |
| chr5 | 179581131 | 179597814 | 1.09E−12 | −43.12640294 | Hypo-methylated | NEPC\|CRPC-Adeno DMRs |
| chr5 | 179604314 | 179604469 | 0.000104758 | −20.69416137 | Hypo-methylated | NEPC\|CRPC-Adeno DMRs |
| chr5 | 179621364 | 179621503 | 0.001802623 | −20.7593985 | Hypo-methylated | NEPC\|CRPC-Adeno DMRs |
| chr5 | 179745870 | 179745934 | 0.001156603 | −21.53157895 | Hypo-methylated | NEPC\|CRPC-Adeno DMRs |
| chr5 | 179771685 | 179774484 | 0.008985252 | −21.90175439 | Hypo-methylated | NEPC\|CRPC-Adeno DMRs |
| chr5 | 179813185 | 179826025 | 1.76E−05 | −22.29042426 | Hypo-methylated | NEPC\|CRPC-Adeno DMRs |
| chr5 | 179839133 | 179840031 | 0.000176134 | −26.82236842 | Hypo-methylated | NEPC\|CRPC-Adeno DMRs |
| chr5 | 180046943 | 180047307 | 0.000477706 | −22.37200378 | Hypo-methylated | NEPC\|CRPC-Adeno DMRs |
| chr5 | 180051729 | 180056041 | 0.005737741 | −20.84230019 | Hypo-methylated | NEPC\|CRPC-Adeno DMRs |
| chr5 | 180487437 | 180495651 | 0.001694183 | −35.69327485 | Hypo-methylated | NEPC\|CRPC-Adeno DMRs |
| chr5 | 180541324 | 180558751 | 8.72E−06 | −20.30473007 | Hypo-methylated | NEPC\|CRPC-Adeno DMRs |
| chr5 | 180579854 | 180581312 | 1.70E−05 | −21.35021721 | Hypo-methylated | NEPC\|CRPC-Adeno DMRs |
| chr5 | 180601471 | 180602019 | 0.006785531 | 21.79078947 | Hyper-methylated | NEPC\|CRPC-Adeno DMRs |
| chr6 | 380009 | 387097 | 2.91E−05 | −39.2166135 | Hypo-methylated | NEPC\|CRPC-Adeno DMRs |
| chr6 | 920943 | 1026676 | 1.39E−08 | −21.3173171 | Hypo-methylated | NEPC\|CRPC-Adeno DMRs |
| chr6 | 1072544 | 1080834 | 2.04E−06 | −23.11078533 | Hypo-methylated | NEPC\|CRPC-Adeno DMRs |
| chr6 | 1091634 | 1169971 | 4.28E−08 | −22.86548112 | Hypo-methylated | NEPC\|CRPC-Adeno DMRs |
| chr6 | 1171332 | 1204780 | 0.002692265 | −23.47569337 | Hypo-methylated | NEPC\|CRPC-Adeno DMRs |
| chr6 | 1218137 | 1311386 | 3.57E−06 | −23.75880713 | Hypo-methylated | NEPC\|CRPC-Adeno DMRs |
| chr6 | 1325258 | 1368287 | 1.15E−05 | −30.48767753 | Hypo-methylated | NEPC\|CRPC-Adeno DMRs |
| chr6 | 3247958 | 3248099 | 0.001694183 | −20.45175439 | Hypo-methylated | NEPC\|CRPC-Adeno DMRs |
| chr6 | 3312949 | 3313253 | 0.001694183 | −42.26111111 | Hypo-methylated | NEPC\|CRPC-Adeno DMRs |
| chr6 | 4609129 | 4609215 | 0.001694183 | −51.94346979 | Hypo-methylated | NEPC\|CRPC-Adeno DMRs |
| chr6 | 6534755 | 6534840 | 0.000324528 | −21.79736842 | Hypo-methylated | NEPC\|CRPC-Adeno DMRs |
| chr6 | 6901786 | 6921548 | 0.001694183 | −29.42330827 | Hypo-methylated | NEPC\|CRPC-Adeno DMRs |
| chr6 | 7051203 | 7051498 | 5.36E−06 | −20.11754386 | Hypo-methylated | NEPC\|CRPC-Adeno DMRs |
| chr6 | 7142066 | 7151210 | 1.65E−05 | −32.35656884 | Hypo-methylated | NEPC\|CRPC-Adeno DMRs |
| chr6 | 7911603 | 7916324 | 0.003327791 | −36.64860681 | Hypo-methylated | NEPC\|CRPC-Adeno DMRs |
| chr6 | 15034147 | 15034388 | 0.003327791 | −34.24130224 | Hypo-methylated | NEPC\|CRPC-Adeno DMRs |
| chr6 | 16231908 | 16234723 | 0.000549237 | 30.56424394 | Hyper-methylated | NEPC\|CRPC-Adeno DMRs |
| chr6 | 16597423 | 16663495 | 7.00E−05 | −26.31060606 | Hypo-methylated | NEPC\|CRPC-Adeno DMRs |
| chr6 | 26225433 | 26233669 | 7.16E−08 | 23.06214273 | Hyper-methylated | NEPC\|CRPC-Adeno DMRs |
| chr6 | 27235154 | 27244420 | 0.001694183 | −27.96274018 | Hypo-methylated | NEPC\|CRPC-Adeno DMRs |
| chr6 | 27507081 | 27507125 | 0.001694183 | 22.48333333 | Hyper-methylated | NEPC\|CRPC-Adeno DMRs |
| chr6 | 28469380 | 28469639 | 0.005737741 | −20.65740741 | Hypo-methylated | NEPC\|CRPC-Adeno DMRs |
| chr6 | 28925778 | 28925944 | 0.001694183 | −28.05874408 | Hypo-methylated | NEPC\|CRPC-Adeno DMRs |
| chr6 | 29004357 | 29097960 | 0.002201952 | −34.40877193 | Hypo-methylated | NEPC\|CRPC-Adeno DMRs |
| chr6 | 30139933 | 30139979 | 1.70E−05 | 22.23368421 | Hyper-methylated | NEPC\|CRPC-Adeno DMRs |
| chr6 | 31079577 | 31088463 | 0.002593231 | −21.8442617 | Hypo-methylated | NEPC\|CRPC-Adeno DMRs |
| chr6 | 31127321 | 31127440 | 0.007136365 | −27.53458647 | Hypo-methylated | NEPC\|CRPC-Adeno DMRs |
| chr6 | 31153818 | 31153913 | 0.001694183 | 34.23606238 | Hyper-methylated | NEPC\|CRPC-Adeno DMRs |
| chr6 | 31516024 | 31516094 | 0.001694183 | −37.28596491 | Hypo-methylated | NEPC\|CRPC-Adeno DMRs |
| chr6 | 31630341 | 31631937 | 0.004750844 | −23.58442982 | Hypo-methylated | NEPC\|CRPC-Adeno DMRs |
| chr6 | 31799374 | 31800092 | 0.005737741 | 26.72913083 | Hyper-methylated | NEPC\|CRPC-Adeno DMRs |
| chr6 | 31869003 | 31869196 | 4.42E−06 | −29.89239035 | Hypo-methylated | NEPC\|CRPC-Adeno DMRs |
| chr6 | 32010328 | 32014958 | 0.000190593 | −29.32598899 | Hypo-methylated | NEPC\|CRPC-Adeno DMRs |
| chr6 | 32017304 | 32055214 | 5.09E−07 | −20.81007653 | Hypo-methylated | NEPC\|CRPC-Adeno DMRs |
| chr6 | 32064904 | 32066461 | 6.10E−05 | −23.97378718 | Hypo-methylated | NEPC\|CRPC-Adeno DMRs |
| chr6 | 32222668 | 32334465 | 4.20E−09 | −34.72389005 | Hypo-methylated | NEPC\|CRPC-Adeno DMRs |
| chr6 | 32441758 | 32452229 | 0.000400293 | −37.43005757 | Hypo-methylated | NEPC\|CRPC-Adeno DMRs |
| chr6 | 33129850 | 33137058 | 0.000414837 | −25.05707741 | Hypo-methylated | NEPC\|CRPC-Adeno DMRs |
| chr6 | 33694845 | 33695935 | 0.009822962 | −31.77889421 | Hypo-methylated | NEPC\|CRPC-Adeno DMRs |
| chr6 | 33798997 | 33806285 | 0.001883547 | 29.24174641 | Hyper-methylated | NEPC\|CRPC-Adeno DMRs |
| chr6 | 33825371 | 33826398 | 0.004750844 | 23.00517962 | Hyper-methylated | NEPC\|CRPC-Adeno DMRs |
| chr6 | 34097690 | 34105786 | 0.003327791 | 22.2208973 | Hyper-methylated | NEPC\|CRPC-Adeno DMRs |
| chr6 | 35479466 | 35480074 | 0.001330041 | 20.53398415 | Hyper-methylated | NEPC\|CRPC-Adeno DMRs |
| chr6 | 35732621 | 35732826 | 8.87E−06 | −27.25141326 | Hypo-methylated | NEPC\|CRPC-Adeno DMRs |
| chr6 | 35944410 | 35944479 | 0.000549237 | −22.55545113 | Hypo-methylated | NEPC\|CRPC-Adeno DMRs |
| chr6 | 36297769 | 36297929 | 0.004750844 | −22.06130461 | Hypo-methylated | NEPC\|CRPC-Adeno DMRs |
| chr6 | 36308454 | 36308550 | 0.003525992 | 23.19824561 | Hyper-methylated | NEPC\|CRPC-Adeno DMRs |
| chr6 | 36808647 | 36808895 | 2.23E−05 | 33.42605994 | Hyper-methylated | NEPC\|CRPC-Adeno DMRs |
| chr6 | 36966633 | 36966850 | 0.001694183 | −38.27293888 | Hypo-methylated | NEPC\|CRPC-Adeno DMRs |
| chr6 | 37027177 | 37027391 | 0.007271222 | −20.91616099 | Hypo-methylated | NEPC\|CRPC-Adeno DMRs |
| chr6 | 37056029 | 37070024 | 0.009458442 | −27.40789474 | Hypo-methylated | NEPC\|CRPC-Adeno DMRs |
| chr6 | 37099083 | 37099308 | 0.007136365 | −26.66461743 | Hypo-methylated | NEPC\|CRPC-Adeno DMRs |
| chr6 | 37616620 | 37618010 | 4.47E−11 | 25.62558993 | Hyper-methylated | NEPC\|CRPC-Adeno DMRs |
| chr6 | 37673065 | 37673164 | 0.000549237 | −21.87928363 | Hypo-methylated | NEPC\|CRPC-Adeno DMRs |
| chr6 | 38683142 | 38683226 | 0.000200814 | 28.21157895 | Hyper-methylated | NEPC\|CRPC-Adeno DMRs |
| chr6 | 39152904 | 39156039 | 0.002976353 | −27.9625731 | Hypo-methylated | NEPC\|CRPC-Adeno DMRs |
| chr6 | 39281155 | 39281986 | 4.39E−26 | −28.4892065 | Hypo-methylated | NEPC\|CRPC-Adeno DMRs |
| chr6 | 40556525 | 40567408 | 0.000193859 | 32.46491228 | Hyper-methylated | NEPC\|CRPC-Adeno DMRs |
| chr6 | 41515524 | 41520612 | 6.22E−07 | −22.56560987 | Hypo-methylated | NEPC\|CRPC-Adeno DMRs |
| chr6 | 41645384 | 41645574 | 4.03E−08 | −22.91719298 | Hypo-methylated | NEPC\|CRPC-Adeno DMRs |
| chr6 | 41691450 | 41701472 | 7.22E−05 | −35.28975318 | Hypo-methylated | NEPC\|CRPC-Adeno DMRs |
| chr6 | 42211020 | 42222590 | 1.46E−05 | 20.48666667 | Hyper-methylated | NEPC\|CRPC-Adeno DMRs |
| chr6 | 43252789 | 43253154 | 2.58E−12 | 36.14350552 | Hyper-methylated | NEPC\|CRPC-Adeno DMRs |
| chr6 | 43691927 | 43701954 | 0.009458442 | 22.59639376 | Hyper-methylated | NEPC\|CRPC-Adeno DMRs |
| chr6 | 43859217 | 43871833 | 0.001249757 | −41.6247076 | Hypo-methylated | NEPC\|CRPC-Adeno DMRs |
| chr6 | 49518155 | 49519263 | 1.68E−16 | −33.59982026 | Hypo-methylated | NEPC\|CRPC-Adeno DMRs |
| chr6 | 51880478 | 51918106 | 0.000324528 | −23.72643189 | Hypo-methylated | NEPC\|CRPC-Adeno DMRs |
| chr6 | 52148092 | 52148147 | 0.003113626 | −34.22270955 | Hypo-methylated | NEPC\|CRPC-Adeno DMRs |
| chr6 | 64150762 | 64152040 | 0.000171929 | −40.23906883 | Hypo-methylated | NEPC\|CRPC-Adeno DMRs |
| chr6 | 67748043 | 68598961 | 3.10E−15 | −25.69795914 | Hypo-methylated | NEPC\|CRPC-Adeno DMRs |
| chr6 | 68824520 | 69073198 | 4.95E−05 | −31.59374966 | Hypo-methylated | NEPC\|CRPC-Adeno DMRs |
| chr6 | 72142128 | 72295260 | 5.17E−06 | −34.35036075 | Hypo-methylated | NEPC\|CRPC-Adeno DMRs |
| chr6 | 72298310 | 72298363 | 0.000168572 | 22.91835512 | Hyper-methylated | NEPC\|CRPC-Adeno DMRs |
| chr6 | 73195959 | 73196049 | 0.001694183 | −26.99074074 | Hypo-methylated | NEPC\|CRPC-Adeno DMRs |
| chr6 | 73446873 | 73686160 | 0.007136365 | −29.79794585 | Hypo-methylated | NEPC\|CRPC-Adeno DMRs |
| chr6 | 74058053 | 74058290 | 0.000581135 | −20.50277778 | Hypo-methylated | NEPC\|CRPC-Adeno DMRs |
| chr6 | 74058740 | 74058815 | 0.000549237 | 21.63492063 | Hyper-methylated | NEPC\|CRPC-Adeno DMRs |
| chr6 | 74063014 | 74063837 | 0.000549237 | −25.36293733 | Hypo-methylated | NEPC\|CRPC-Adeno DMRs |
| chr6 | 74079707 | 74095500 | 3.15E−05 | −21.10204365 | Hypo-methylated | NEPC\|CRPC-Adeno DMRs |
| chr6 | 75799863 | 75799927 | 0.001694183 | −34.3537037 | Hypo-methylated | NEPC\|CRPC-Adeno DMRs |
| chr6 | 76022149 | 76029739 | 0.002201952 | −21.98854167 | Hypo-methylated | NEPC\|CRPC-Adeno DMRs |
| chr6 | 78172277 | 78172873 | 2.71E−07 | 27.93775017 | Hyper-methylated | NEPC\|CRPC-Adeno DMRs |
| chr6 | 78372148 | 79244679 | 7.90E−06 | −28.25903182 | Hypo-methylated | NEPC\|CRPC-Adeno DMRs |
| chr6 | 85483168 | 85484776 | 6.94E−28 | 28.62015171 | Hyper-methylated | NEPC\|CRPC-Adeno DMRs |
| chr6 | 90597327 | 90597525 | 1.06E−08 | 20.1827957 | Hyper-methylated | NEPC\|CRPC-Adeno DMRs |
| chr6 | 94183722 | 94347277 | 0.000184724 | 21.32184874 | Hyper-methylated | NEPC\|CRPC-Adeno DMRs |
| chr6 | 96463182 | 96464064 | 4.24E−06 | 26.82815904 | Hyper-methylated | NEPC\|CRPC-Adeno DMRs |
| chr6 | 97440107 | 97440399 | 0.005737741 | −36.85833333 | Hypo-methylated | NEPC\|CRPC-Adeno DMRs |
| chr6 | 97763759 | 98183523 | 0.001179842 | −29.05151679 | Hypo-methylated | NEPC\|CRPC-Adeno DMRs |
| chr6 | 99290467 | 99291654 | 3.19E−10 | 25.49718992 | Hyper-methylated | NEPC\|CRPC-Adeno DMRs |
| chr6 | 100050801 | 100051345 | 2.35E−08 | 25.43145833 | Hyper-methylated | NEPC\|CRPC-Adeno DMRs |
| chr6 | 100051516 | 100051798 | 1.52E−13 | 26.67049062 | Hyper-methylated | NEPC\|CRPC-Adeno DMRs |
| chr6 | 100053497 | 100053559 | 0.005737741 | 20.41486928 | Hyper-methylated | NEPC\|CRPC-Adeno DMRs |
| chr6 | 100441301 | 100441849 | 2.20E−12 | 26.41419011 | Hyper-methylated | NEPC\|CRPC-Adeno DMRs |
| chr6 | 106442088 | 106442130 | 5.50E−05 | 43.72367725 | Hyper-methylated | NEPC\|CRPC-Adeno DMRs |
| chr6 | 108685423 | 108685477 | 0.001694183 | −46.02698413 | Hypo-methylated | NEPC\|CRPC-Adeno DMRs |
| chr6 | 110720729 | 110721547 | 6.50E−07 | 30.20072239 | Hyper-methylated | NEPC\|CRPC-Adeno DMRs |
| chr6 | 117003416 | 117003447 | 0.001430236 | −33.85073529 | Hypo-methylated | NEPC\|CRPC-Adeno DMRs |
| chr6 | 117336184 | 117566170 | 0.000348917 | −24.95925756 | Hypo-methylated | NEPC\|CRPC-Adeno DMRs |
| chr6 | 117650809 | 117774415 | 3.72E−05 | −27.81867052 | Hypo-methylated | NEPC\|CRPC-Adeno DMRs |
| chr6 | 118071921 | 118142737 | 0.000332311 | −32.58397034 | Hypo-methylated | NEPC\|CRPC-Adeno DMRs |
| chr6 | 121192520 | 121292329 | 0.0077394 | −22.9501462 | Hypo-methylated | NEPC\|CRPC-Adeno DMRs |
| chr6 | 122051071 | 122601919 | 6.76E−10 | −31.22322451 | Hypo-methylated | NEPC\|CRPC-Adeno DMRs |
| chr6 | 125743175 | 125767279 | 0.003113626 | 27.92060185 | Hyper-methylated | NEPC\|CRPC-Adeno DMRs |
| chr6 | 130135971 | 130319227 | 7.27E−07 | −25.94394399 | Hypo-methylated | NEPC\|CRPC-Adeno DMRs |
| chr6 | 131076017 | 131091928 | 0.000176134 | −23.28289474 | Hypo-methylated | NEPC\|CRPC-Adeno DMRs |
| chr6 | 131205293 | 131343090 | 2.48E−05 | −20.65603715 | Hypo-methylated | NEPC\|CRPC-Adeno DMRs |
| chr6 | 131615473 | 131693099 | 2.71E−06 | −22.20950501 | Hypo-methylated | NEPC\|CRPC-Adeno DMRs |
| chr6 | 132982727 | 133075066 | 0.003890964 | −29.79025413 | Hypo-methylated | NEPC\|CRPC-Adeno DMRs |
| chr6 | 133561993 | 133563178 | 2.72E−20 | 34.25815513 | Hyper-methylated | NEPC\|CRPC-Adeno DMRs |
| chr6 | 134210997 | 134211187 | 0.000328065 | 26.28901041 | Hyper-methylated | NEPC\|CRPC-Adeno DMRs |
| chr6 | 134213989 | 134217387 | 1.13E−05 | 28.18877971 | Hyper-methylated | NEPC\|CRPC-Adeno DMRs |
| chr6 | 134587469 | 134589635 | 0.008985252 | −30.30441845 | Hypo-methylated | NEPC\|CRPC-Adeno DMRs |
| chr6 | 137662985 | 137663069 | 0.001694183 | −31.84912281 | Hypo-methylated | NEPC\|CRPC-Adeno DMRs |
| chr6 | 139003913 | 139004094 | 6.31E−05 | −30.31959412 | Hypo-methylated | NEPC\|CRPC-Adeno DMRs |
| chr6 | 146550415 | 146745382 | 0.001545937 | −24.0022742 | Hypo-methylated | NEPC\|CRPC-Adeno DMRs |
| chr6 | 149196103 | 149207698 | 0.001694183 | −49.44546261 | Hypo-methylated | NEPC\|CRPC-Adeno DMRs |
| chr6 | 149771897 | 149772344 | 1.04E−21 | −26.17262824 | Hypo-methylated | NEPC\|CRPC-Adeno DMRs |
| chr6 | 149868167 | 149868236 | 0.000176134 | −34.15972222 | Hypo-methylated | NEPC\|CRPC-Adeno DMRs |
| chr6 | 150161647 | 150173719 | 0.003113626 | −23.85284392 | Hypo-methylated | NEPC\|CRPC-Adeno DMRs |
| chr6 | 150226728 | 150231840 | 0.000549237 | −20.91428571 | Hypo-methylated | NEPC\|CRPC-Adeno DMRs |
| chr6 | 151560872 | 151560928 | 0.001694183 | 23.8462963 | Hyper-methylated | NEPC\|CRPC-Adeno DMRs |
| chr6 | 155316153 | 155316317 | 0.005972124 | 20.64532164 | Hyper-methylated | NEPC\|CRPC-Adeno DMRs |
| chr6 | 155577670 | 155577944 | 0.006785531 | 25.75248968 | Hyper-methylated | NEPC\|CRPC-Adeno DMRs |
| chr6 | 156919689 | 156919819 | 0.000549237 | −26.87518797 | Hypo-methylated | NEPC\|CRPC-Adeno DMRs |
| chr6 | 157301740 | 157301884 | 0.000549237 | −27.48681999 | Hypo-methylated | NEPC\|CRPC-Adeno DMRs |
| chr6 | 157549697 | 157550558 | 0.000381376 | −44.41951754 | Hypo-methylated | NEPC\|CRPC-Adeno DMRs |
| chr6 | 157638537 | 157695368 | 0.000614774 | −35.25709208 | Hypo-methylated | NEPC\|CRPC-Adeno DMRs |
| chr6 | 158142482 | 158143443 | 0.001694183 | −21.9917842 | Hypo-methylated | NEPC\|CRPC-Adeno DMRs |
| chr6 | 158402097 | 158402130 | 0.001694183 | 42.13095238 | Hyper-methylated | NEPC\|CRPC-Adeno DMRs |
| chr6 | 159052282 | 159052968 | 0.000176134 | 35.46460376 | Hyper-methylated | NEPC\|CRPC-Adeno DMRs |
| chr6 | 159086455 | 159086574 | 5.50E−05 | −27.42098765 | Hypo-methylated | NEPC\|CRPC-Adeno DMRs |
| chr6 | 159178146 | 159178308 | 0.000549237 | −26.66904762 | Hypo-methylated | NEPC\|CRPC-Adeno DMRs |
| chr6 | 159179591 | 159180045 | 5.75E−05 | −48.55696895 | Hypo-methylated | NEPC\|CRPC-Adeno DMRs |
| chr6 | 160647285 | 160768460 | 1.98E−16 | −31.00488046 | Hypo-methylated | NEPC\|CRPC-Adeno DMRs |
| chr6 | 160770301 | 160908523 | 1.24E−10 | −26.58055134 | Hypo-methylated | NEPC\|CRPC-Adeno DMRs |
| chr6 | 160977157 | 161056077 | 9.09E−05 | −27.28460039 | Hypo-methylated | NEPC\|CRPC-Adeno DMRs |
| chr6 | 161106413 | 161188054 | 5.17E−06 | −28.52580409 | Hypo-methylated | NEPC\|CRPC-Adeno DMRs |
| chr6 | 161188085 | 161188130 | 5.50E−05 | 38.73014133 | Hyper-methylated | NEPC\|CRPC-Adeno DMRs |
| chr6 | 161188265 | 161188350 | 1.02E−13 | 25.70012531 | Hyper-methylated | NEPC\|CRPC-Adeno DMRs |
| chr6 | 162192054 | 163130689 | 2.69E−45 | −44.66560673 | Hypo-methylated | NEPC\|CRPC-Adeno DMRs |
| chr6 | 163185201 | 163558674 | 1.07E−16 | −38.89754784 | Hypo-methylated | NEPC\|CRPC-Adeno DMRs |
| chr6 | 163744319 | 163749730 | 3.32E−05 | −23.84523724 | Hypo-methylated | NEPC\|CRPC-Adeno DMRs |
| chr6 | 163786097 | 163809070 | 0.001287702 | −25.28347903 | Hypo-methylated | NEPC\|CRPC-Adeno DMRs |
| chr6 | 164051754 | 164051943 | 1.20E−06 | 20.89901063 | Hyper-methylated | NEPC\|CRPC-Adeno DMRs |
| chr6 | 164083974 | 164215623 | 1.12E−06 | −26.32551847 | Hypo-methylated | NEPC\|CRPC-Adeno DMRs |
| chr6 | 166233001 | 166233231 | 1.22E−05 | 39.48162072 | Hyper-methylated | NEPC\|CRPC-Adeno DMRs |
| chr6 | 166267976 | 166268220 | 1.89E−05 | 24.36348684 | Hyper-methylated | NEPC\|CRPC-Adeno DMRs |
| chr6 | 166402580 | 166402611 | 0.000549237 | −20.02255639 | Hypo-methylated | NEPC\|CRPC-Adeno DMRs |
| chr6 | 167263366 | 167263443 | 0.003525992 | −26.17592593 | Hypo-methylated | NEPC\|CRPC-Adeno DMRs |
| chr6 | 167317683 | 167317839 | 0.001694183 | −37.67755991 | Hypo-methylated | NEPC\|CRPC-Adeno DMRs |
| chr6 | 167662768 | 167663357 | 0.001694183 | −27.73148148 | Hypo-methylated | NEPC\|CRPC-Adeno DMRs |
| chr6 | 168461811 | 168463153 | 0.001600753 | −36.86793592 | Hypo-methylated | NEPC\|CRPC-Adeno DMRs |
| chr6 | 168490497 | 168490753 | 4.65E−07 | −42.52564103 | Hypo-methylated | NEPC\|CRPC-Adeno DMRs |
| chr6 | 168532323 | 168534097 | 3.03E−06 | 38.88784924 | Hyper-methylated | NEPC\|CRPC-Adeno DMRs |
| chr6 | 168595964 | 168599827 | 0.001694183 | −33.59880174 | Hypo-methylated | NEPC\|CRPC-Adeno DMRs |
| chr6 | 168678236 | 168678443 | 0.003113626 | −24.14444444 | Hypo-methylated | NEPC\|CRPC-Adeno DMRs |
| chr6 | 168774238 | 168777827 | 9.94E−05 | −30.09728175 | Hypo-methylated | NEPC\|CRPC-Adeno DMRs |
| chr6 | 168927073 | 168927139 | 0.001694183 | −39.03518519 | Hypo-methylated | NEPC\|CRPC-Adeno DMRs |
| chr6 | 168983447 | 168983516 | 0.000176134 | −20.88888889 | Hypo-methylated | NEPC\|CRPC-Adeno DMRs |
| chr6 | 169017534 | 169020490 | 0.001694183 | 20.54945534 | Hyper-methylated | NEPC\|CRPC-Adeno DMRs |
| chr6 | 169267548 | 169267776 | 0.000926684 | −20.20575163 | Hypo-methylated | NEPC\|CRPC-Adeno DMRs |
| chr6 | 169271008 | 169271390 | 0.000488426 | −34.68547009 | Hypo-methylated | NEPC\|CRPC-Adeno DMRs |
| chr6 | 170224164 | 170224458 | 0.001694183 | −34.65947712 | Hypo-methylated | NEPC\|CRPC-Adeno DMRs |
| chr6 | 170396258 | 170396371 | 0.000549237 | −44.08888889 | Hypo-methylated | NEPC\|CRPC-Adeno DMRs |
| chr6 | 170418159 | 170418327 | 4.65E−07 | 20.58706098 | Hyper-methylated | NEPC\|CRPC-Adeno DMRs |
| chr6 | 170449348 | 170449421 | 0.001694183 | −46.28333333 | Hypo-methylated | NEPC\|CRPC-Adeno DMRs |
| chr6 | 170468244 | 170468763 | 0.00533697 | −28.34436508 | Hypo-methylated | NEPC\|CRPC-Adeno DMRs |
| chr6 | 170577679 | 170578638 | 0.000220189 | −43.35607688 | Hypo-methylated | NEPC\|CRPC-Adeno DMRs |
| chr7 | 98021 | 117929 | 4.65E−07 | −28.51790373 | Hypo-methylated | NEPC\|CRPC-Adeno DMRs |
| chr7 | 139180 | 139734 | 1.98E−05 | −21.85387989 | Hypo-methylated | NEPC\|CRPC-Adeno DMRs |
| chr7 | 162710 | 162891 | 1.70E−05 | −27.49614035 | Hypo-methylated | NEPC\|CRPC-Adeno DMRs |
| chr7 | 168126 | 173660 | 1.10E−10 | −20.44578345 | Hypo-methylated | NEPC\|CRPC-Adeno DMRs |
| chr7 | 186539 | 186625 | 0.003525992 | −20.4508215 | Hypo-methylated | NEPC\|CRPC-Adeno DMRs |
| chr7 | 388177 | 388516 | 0.000148603 | −22.96399068 | Hypo-methylated | NEPC\|CRPC-Adeno DMRs |
| chr7 | 461264 | 462764 | 1.70E−09 | −28.04467959 | Hypo-methylated | NEPC\|CRPC-Adeno DMRs |
| chr7 | 502171 | 505484 | 1.25E−22 | −20.6690523 | Hypo-methylated | NEPC\|CRPC-Adeno DMRs |
| chr7 | 511489 | 511570 | 1.70E−05 | −22.72350877 | Hypo-methylated | NEPC\|CRPC-Adeno DMRs |
| chr7 | 519881 | 525711 | 1.98E−05 | −30.04869867 | Hypo-methylated | NEPC\|CRPC-Adeno DMRs |
| chr7 | 531628 | 532623 | 1.63E−05 | −27.01310141 | Hypo-methylated | NEPC\|CRPC-Adeno DMRs |
| chr7 | 569903 | 570042 | 3.46E−09 | −33.47004917 | Hypo-methylated | NEPC\|CRPC-Adeno DMRs |
| chr7 | 573034 | 576711 | 3.68E−08 | −26.15599437 | Hypo-methylated | NEPC\|CRPC-Adeno DMRs |
| chr7 | 731360 | 733947 | 4.34E−06 | −21.49363794 | Hypo-methylated | NEPC\|CRPC-Adeno DMRs |
| chr7 | 943496 | 943677 | 5.17E−06 | −26.22501899 | Hypo-methylated | NEPC\|CRPC-Adeno DMRs |
| chr7 | 975316 | 975409 | 5.17E−06 | −28.82902399 | Hypo-methylated | NEPC\|CRPC-Adeno DMRs |
| chr7 | 1036915 | 1037386 | 1.97E−05 | −21.90503326 | Hypo-methylated | NEPC\|CRPC-Adeno DMRs |
| chr7 | 1116099 | 1116660 | 4.55E−08 | 26.47100765 | Hyper-methylated | NEPC\|CRPC-Adeno DMRs |
| chr7 | 1140343 | 1141234 | 0.002094266 | −20.203771 | Hypo-methylated | NEPC\|CRPC-Adeno DMRs |
| chr7 | 1163153 | 1170455 | 1.01E−07 | 20.35215718 | Hyper-methylated | NEPC\|CRPC-Adeno DMRs |
| chr7 | 1297674 | 1300321 | 3.26E−07 | −22.72610939 | Hypo-methylated | NEPC\|CRPC-Adeno DMRs |
| chr7 | 1308374 | 1309519 | 3.39E−07 | −28.25817853 | Hypo-methylated | NEPC\|CRPC-Adeno DMRs |
| chr7 | 1351824 | 1362126 | 5.19E−09 | −23.90198515 | Hypo-methylated | NEPC\|CRPC-Adeno DMRs |
| chr7 | 1403804 | 1408569 | 0.000535455 | −20.2246171 | Hypo-methylated | NEPC\|CRPC-Adeno DMRs |
| chr7 | 1409096 | 1409194 | 0.000581135 | 26.57816155 | Hyper-methylated | NEPC\|CRPC-Adeno DMRs |
| chr7 | 1447698 | 1447784 | 5.50E−05 | −57.72644574 | Hypo-methylated | NEPC\|CRPC-Adeno DMRs |
| chr7 | 1486267 | 1486774 | 5.50E−05 | −35.45552307 | Hypo-methylated | NEPC\|CRPC-Adeno DMRs |
| chr7 | 1501203 | 1501306 | 2.58E−07 | 30.06159411 | Hyper-methylated | NEPC\|CRPC-Adeno DMRs |
| chr7 | 1572925 | 1574017 | 0.000112114 | 21.47932174 | Hyper-methylated | NEPC\|CRPC-Adeno DMRs |
| chr7 | 1625289 | 1627198 | 2.12E−12 | −29.82460375 | Hypo-methylated | NEPC\|CRPC-Adeno DMRs |
| chr7 | 1638032 | 1639465 | 0.000324528 | −20.00967492 | Hypo-methylated | NEPC\|CRPC-Adeno DMRs |
| chr7 | 1764914 | 1765159 | 5.50E−05 | −34.2920078 | Hypo-methylated | NEPC\|CRPC-Adeno DMRs |
| chr7 | 1786426 | 1786633 | 1.43E−09 | −24.62849253 | Hypo-methylated | NEPC\|CRPC-Adeno DMRs |
| chr7 | 1798978 | 1799229 | 0.000151874 | −31.57120196 | Hypo-methylated | NEPC\|CRPC-Adeno DMRs |
| chr7 | 1799972 | 1801577 | 0.000184724 | −20.84769563 | Hypo-methylated | NEPC\|CRPC-Adeno DMRs |
| chr7 | 1972363 | 1973580 | 1.22E−05 | −37.9066237 | Hypo-methylated | NEPC\|CRPC-Adeno DMRs |
| chr7 | 1988131 | 1988157 | 0.007478734 | 29.64692982 | Hyper-methylated | NEPC\|CRPC-Adeno DMRs |
| chr7 | 2006578 | 2007507 | 4.03E−08 | −50.03189084 | Hypo-methylated | NEPC\|CRPC-Adeno DMRs |
| chr7 | 2030225 | 2035752 | 3.70E−06 | −21.53736424 | Hypo-methylated | NEPC\|CRPC-Adeno DMRs |
| chr7 | 2091247 | 2092597 | 3.81E−05 | −35.92921831 | Hypo-methylated | NEPC\|CRPC-Adeno DMRs |
| chr7 | 2147551 | 2149019 | 8.71E−07 | −23.28644708 | Hypo-methylated | NEPC\|CRPC-Adeno DMRs |
| chr7 | 2161050 | 2161107 | 0.000953892 | −31.75082237 | Hypo-methylated | NEPC\|CRPC-Adeno DMRs |
| chr7 | 2183803 | 2184317 | 0.002976353 | −30.9028655 | Hypo-methylated | NEPC\|CRPC-Adeno DMRs |
| chr7 | 2544926 | 2545165 | 1.70E−05 | 30.98345029 | Hyper-methylated | NEPC\|CRPC-Adeno DMRs |
| chr7 | 2705046 | 2706334 | 8.77E−06 | −27.42248804 | Hypo-methylated | NEPC\|CRPC-Adeno DMRs |
| chr7 | 2802248 | 2803117 | 4.74E−05 | 24.76065831 | Hyper-methylated | NEPC\|CRPC-Adeno DMRs |
| chr7 | 2953704 | 2953931 | 4.66E−05 | −32.14649123 | Hypo-methylated | NEPC\|CRPC-Adeno DMRs |
| chr7 | 4031683 | 4047370 | 0.005574264 | −23.16204147 | Hypo-methylated | NEPC\|CRPC-Adeno DMRs |
| chr7 | 4238898 | 4238967 | 0.001694183 | 27.15126706 | Hyper-methylated | NEPC\|CRPC-Adeno DMRs |
| chr7 | 4831773 | 4831965 | 1.45E−08 | 35.25098688 | Hyper-methylated | NEPC\|CRPC-Adeno DMRs |
| chr7 | 4896490 | 4896642 | 0.001694183 | −43.76929825 | Hypo-methylated | NEPC\|CRPC-Adeno DMRs |
| chr7 | 4901427 | 4901504 | 6.03E−06 | 26.68003063 | Hyper-methylated | NEPC\|CRPC-Adeno DMRs |
| chr7 | 5122981 | 5123129 | 8.34E−06 | −21.24732665 | Hypo-methylated | NEPC\|CRPC-Adeno DMRs |
| chr7 | 5269948 | 5277035 | 0.004484018 | 27.6063606 | Hyper-methylated | NEPC\|CRPC-Adeno DMRs |
| chr7 | 5571994 | 5572281 | 0.000176134 | 58.83446315 | Hyper-methylated | NEPC\|CRPC-Adeno DMRs |
| chr7 | 6653117 | 6655380 | 1.09E−08 | −20.46297397 | Hypo-methylated | NEPC\|CRPC-Adeno DMRs |
| chr7 | 6894841 | 6910747 | 3.72E−05 | −22.36660819 | Hypo-methylated | NEPC\|CRPC-Adeno DMRs |
| chr7 | 14942275 | 14942568 | 0.000100851 | −22.76273259 | Hypo-methylated | NEPC\|CRPC-Adeno DMRs |
| chr7 | 16627263 | 16627563 | 5.17E−06 | −37.35151515 | Hypo-methylated | NEPC\|CRPC-Adeno DMRs |
| chr7 | 21985032 | 21985817 | 4.29E−23 | −20.50324465 | Hypo-methylated | NEPC\|CRPC-Adeno DMRs |
| chr7 | 22473691 | 22485269 | 0.001065261 | −33.33907044 | Hypo-methylated | NEPC\|CRPC-Adeno DMRs |
| chr7 | 23513686 | 23514114 | 7.64E−11 | 29.63961782 | Hyper-methylated | NEPC\|CRPC-Adeno DMRs |
| chr7 | 24746385 | 24775234 | 6.23E−07 | −24.75803664 | Hypo-methylated | NEPC\|CRPC-Adeno DMRs |
| chr7 | 26439818 | 26440049 | 1.70E−05 | −38.2158111 | Hypo-methylated | NEPC\|CRPC-Adeno DMRs |
| chr7 | 26667541 | 26667827 | 1.43E−05 | −47.45877976 | Hypo-methylated | NEPC\|CRPC-Adeno DMRs |
| chr7 | 27138615 | 27138758 | 0.000176134 | −22.11076389 | Hypo-methylated | NEPC\|CRPC-Adeno DMRs |
| chr7 | 29370958 | 29390448 | 0.000667554 | −29.61643963 | Hypo-methylated | NEPC\|CRPC-Adeno DMRs |
| chr7 | 29847139 | 29847207 | 1.55E−06 | 26.36096491 | Hyper-methylated | NEPC\|CRPC-Adeno DMRs |
| chr7 | 30509913 | 30510071 | 0.001694183 | −55.98412698 | Hypo-methylated | NEPC\|CRPC-Adeno DMRs |
| chr7 | 32109943 | 32110909 | 2.03E−14 | 21.86596629 | Hyper-methylated | NEPC\|CRPC-Adeno DMRs |
| chr7 | 32337965 | 32338105 | 7.06E−06 | 27.35464181 | Hyper-methylated | NEPC\|CRPC-Adeno DMRs |
| chr7 | 33774146 | 33794990 | 0.001694183 | −36.79699074 | Hypo-methylated | NEPC\|CRPC-Adeno DMRs |
| chr7 | 35293088 | 35294024 | 7.26E−15 | 21.08101678 | Hyper-methylated | NEPC\|CRPC-Adeno DMRs |
| chr7 | 35295107 | 35295185 | 0.001694183 | 35.84756335 | Hyper-methylated | NEPC\|CRPC-Adeno DMRs |
| chr7 | 35297355 | 35298144 | 2.04E−10 | 24.12089977 | Hyper-methylated | NEPC\|CRPC-Adeno DMRs |
| chr7 | 36320670 | 36320934 | 5.50E−05 | −37.13281734 | Hypo-methylated | NEPC\|CRPC-Adeno DMRs |
| chr7 | 37331946 | 37385316 | 0.000716333 | −21.19096816 | Hypo-methylated | NEPC\|CRPC-Adeno DMRs |
| chr7 | 37430881 | 37484208 | 4.58E−05 | −36.78493027 | Hypo-methylated | NEPC\|CRPC-Adeno DMRs |
| chr7 | 39015634 | 39015684 | 0.000515039 | 35.36695906 | Hyper-methylated | NEPC\|CRPC-Adeno DMRs |
| chr7 | 39926086 | 39964562 | 0.001274051 | −30.42906973 | Hypo-methylated | NEPC\|CRPC-Adeno DMRs |
| chr7 | 40771513 | 40949002 | 1.42E−05 | −24.80588235 | Hypo-methylated | NEPC\|CRPC-Adeno DMRs |
| chr7 | 41086076 | 41132732 | 5.17E−06 | −42.73618695 | Hypo-methylated | NEPC\|CRPC-Adeno DMRs |
| chr7 | 42426194 | 42508085 | 1.99E−05 | −24.13349489 | Hypo-methylated | NEPC\|CRPC-Adeno DMRs |
| chr7 | 42561891 | 42903008 | 2.06E−06 | −30.53066362 | Hypo-methylated | NEPC\|CRPC-Adeno DMRs |
| chr7 | 44080363 | 44080556 | 1.22E−05 | −24.76478697 | Hypo-methylated | NEPC\|CRPC-Adeno DMRs |
| chr7 | 44126931 | 44139802 | 0.004705671 | −20.53388717 | Hypo-methylated | NEPC\|CRPC-Adeno DMRs |
| chr7 | 44184906 | 44186013 | 1.42E−06 | 28.87002755 | Hyper-methylated | NEPC\|CRPC-Adeno DMRs |
| chr7 | 44806979 | 44807422 | 0.000398142 | −32.71807549 | Hypo-methylated | NEPC\|CRPC-Adeno DMRs |
| chr7 | 45188077 | 45188125 | 0.009458442 | −21.66140351 | Hypo-methylated | NEPC\|CRPC-Adeno DMRs |
| chr7 | 45756556 | 45761426 | 0.000549237 | −32.43986156 | Hypo-methylated | NEPC\|CRPC-Adeno DMRs |
| chr7 | 45980036 | 46002378 | 0.006890179 | 20.33518519 | Hyper-methylated | NEPC\|CRPC-Adeno DMRs |
| chr7 | 46643054 | 46721722 | 0.002927544 | 29.52222222 | Hyper-methylated | NEPC\|CRPC-Adeno DMRs |
| chr7 | 47571357 | 47576935 | 2.54E−12 | −21.5275848 | Hypo-methylated | NEPC\|CRPC-Adeno DMRs |
| chr7 | 47783603 | 47783645 | 0.000832692 | −25.76339286 | Hypo-methylated | NEPC\|CRPC-Adeno DMRs |
| chr7 | 47878999 | 47879110 | 0.000224262 | −23.43659539 | Hypo-methylated | NEPC\|CRPC-Adeno DMRs |
| chr7 | 48178734 | 48241612 | 0.000341319 | −27.77574658 | Hypo-methylated | NEPC\|CRPC-Adeno DMRs |
| chr7 | 49813013 | 49813981 | 5.85E−33 | 24.51054198 | Hyper-methylated | NEPC\|CRPC-Adeno DMRs |
| chr7 | 50343250 | 50344453 | 1.24E−42 | 31.836395 | Hyper-methylated | NEPC\|CRPC-Adeno DMRs |
| chr7 | 50538740 | 50541014 | 0.001694183 | −35.38267544 | Hypo-methylated | NEPC\|CRPC-Adeno DMRs |
| chr7 | 50628625 | 50645521 | 0.001076438 | −39.43436797 | Hypo-methylated | NEPC\|CRPC-Adeno DMRs |
| chr7 | 51210128 | 51248111 | 0.006847068 | 41.94803676 | Hyper-methylated | NEPC\|CRPC-Adeno DMRs |
| chr7 | 55507332 | 55507385 | 0.000176134 | −48.26915205 | Hypo-methylated | NEPC\|CRPC-Adeno DMRs |
| chr7 | 56242565 | 56242873 | 1.48E−07 | 29.56511716 | Hyper-methylated | NEPC\|CRPC-Adeno DMRs |
| chr7 | 56242964 | 56243315 | 0.000209019 | 22.70106386 | Hyper-methylated | NEPC\|CRPC-Adeno DMRs |
| chr7 | 56515745 | 56515773 | 0.002976353 | 21.16766917 | Hyper-methylated | NEPC\|CRPC-Adeno DMRs |
| chr7 | 62514623 | 62514749 | 0.00013534 | −24.38822612 | Hypo-methylated | NEPC\|CRPC-Adeno DMRs |
| chr7 | 63033542 | 63080718 | 0.000549237 | −21.75166372 | Hypo-methylated | NEPC\|CRPC-Adeno DMRs |
| chr7 | 63392167 | 63423654 | 1.46E−05 | −27.3291205 | Hypo-methylated | NEPC\|CRPC-Adeno DMRs |
| chr7 | 63775219 | 63903300 | 0.000522292 | −23.27074167 | Hypo-methylated | NEPC\|CRPC-Adeno DMRs |
| chr7 | 64035306 | 64035529 | 3.73E−07 | 27.91159701 | Hyper-methylated | NEPC\|CRPC-Adeno DMRs |
| chr7 | 65879286 | 65894441 | 0.000926684 | −33.99538012 | Hypo-methylated | NEPC\|CRPC-Adeno DMRs |
| chr7 | 67016138 | 67016209 | 0.000549237 | −41.44897959 | Hypo-methylated | NEPC\|CRPC-Adeno DMRs |
| chr7 | 67935792 | 67936100 | 0.001694183 | −38.15175439 | Hypo-methylated | NEPC\|CRPC-Adeno DMRs |
| chr7 | 68442589 | 68460614 | 0.009285355 | −31.69861111 | Hypo-methylated | NEPC\|CRPC-Adeno DMRs |
| chr7 | 70194169 | 70195030 | 0.000209051 | −38.52631579 | Hypo-methylated | NEPC\|CRPC-Adeno DMRs |
| chr7 | 70543193 | 70571576 | 0.000109727 | −24.51994288 | Hypo-methylated | NEPC\|CRPC-Adeno DMRs |
| chr7 | 70598187 | 71040669 | 3.44E−23 | −28.85128178 | Hypo-methylated | NEPC\|CRPC-Adeno DMRs |
| chr7 | 71062183 | 71190948 | 3.79E−10 | −29.87299605 | Hypo-methylated | NEPC\|CRPC-Adeno DMRs |
| chr7 | 71597750 | 71788886 | 1.15E−06 | −22.92652133 | Hypo-methylated | NEPC\|CRPC-Adeno DMRs |
| chr7 | 71897417 | 71910527 | 7.00E−05 | −36.82478031 | Hypo-methylated | NEPC\|CRPC-Adeno DMRs |
| chr7 | 73120427 | 73121745 | 0.005972702 | −23.51048949 | Hypo-methylated | NEPC\|CRPC-Adeno DMRs |
| chr7 | 73130447 | 73130663 | 0.001694183 | −24.36789474 | Hypo-methylated | NEPC\|CRPC-Adeno DMRs |
| chr7 | 73292628 | 73293839 | 0.000291618 | −31.06161616 | Hypo-methylated | NEPC\|CRPC-Adeno DMRs |
| chr7 | 73418548 | 73425014 | 0.00013534 | −21.38034531 | Hypo-methylated | NEPC\|CRPC-Adeno DMRs |
| chr7 | 73444203 | 73448630 | 1.53E−09 | −34.02355315 | Hypo-methylated | NEPC\|CRPC-Adeno DMRs |
| chr7 | 73877077 | 73877186 | 0.000549237 | −38.23776107 | Hypo-methylated | NEPC\|CRPC-Adeno DMRs |
| chr7 | 73928945 | 73929137 | 5.50E−05 | −30.19239766 | Hypo-methylated | NEPC\|CRPC-Adeno DMRs |
| chr7 | 75317537 | 75318296 | 1.83E−11 | −46.11286929 | Hypo-methylated | NEPC\|CRPC-Adeno DMRs |
| chr7 | 75428466 | 75430067 | 0.001549499 | −31.85328947 | Hypo-methylated | NEPC\|CRPC-Adeno DMRs |
| chr7 | 75889320 | 75896730 | 8.30E−08 | 36.39937221 | Hyper-methylated | NEPC\|CRPC-Adeno DMRs |
| chr7 | 77759060 | 77781012 | 0.002094266 | −25.62241715 | Hypo-methylated | NEPC\|CRPC-Adeno DMRs |
| chr7 | 80882862 | 80972780 | 0.000291618 | −21.78708134 | Hypo-methylated | NEPC\|CRPC-Adeno DMRs |
| chr7 | 88055452 | 88361379 | 0.000100851 | −23.3035807 | Hypo-methylated | NEPC\|CRPC-Adeno DMRs |
| chr7 | 89402663 | 89699986 | 0.000412306 | −33.21182586 | Hypo-methylated | NEPC\|CRPC-Adeno DMRs |
| chr7 | 92224032 | 92238214 | 0.009167638 | −25.32917875 | Hypo-methylated | NEPC\|CRPC-Adeno DMRs |
| chr7 | 92556126 | 92556358 | 0.001694183 | −29.33085655 | Hypo-methylated | NEPC\|CRPC-Adeno DMRs |
| chr7 | 93027070 | 93179659 | 0.000711935 | −39.84444444 | Hypo-methylated | NEPC\|CRPC-Adeno DMRs |
| chr7 | 93215075 | 93412547 | 0.000247501 | −23.22734832 | Hypo-methylated | NEPC\|CRPC-Adeno DMRs |
| chr7 | 93730217 | 93808002 | 0.000586307 | −21.47168435 | Hypo-methylated | NEPC\|CRPC-Adeno DMRs |
| chr7 | 96679152 | 96693910 | 0.00203966 | 20.39064327 | Hyper-methylated | NEPC\|CRPC-Adeno DMRs |
| chr7 | 97835013 | 97835047 | 0.000549237 | −30.93055556 | Hypo-methylated | NEPC\|CRPC-Adeno DMRs |
| chr7 | 100074961 | 100075551 | 2.34E−07 | 31.55237793 | Hyper-methylated | NEPC\|CRPC-Adeno DMRs |
| chr7 | 100179198 | 100179430 | 5.50E−05 | 22.38992702 | Hyper-methylated | NEPC\|CRPC-Adeno DMRs |
| chr7 | 100202591 | 100203556 | 5.55E−09 | −22.03045273 | Hypo-methylated | NEPC\|CRPC-Adeno DMRs |
| chr7 | 100243689 | 100244361 | 1.77E−05 | 25.94620587 | Hyper-methylated | NEPC\|CRPC-Adeno DMRs |
| chr7 | 100417332 | 100417781 | 0.000549237 | −20.95118153 | Hypo-methylated | NEPC\|CRPC-Adeno DMRs |
| chr7 | 100420198 | 100420392 | 1.17E−08 | −28.21832707 | Hypo-methylated | NEPC\|CRPC-Adeno DMRs |
| chr7 | 100898266 | 100925400 | 7.04E−08 | −30.81060825 | Hypo-methylated | NEPC\|CRPC-Adeno DMRs |
| chr7 | 101047233 | 101079065 | 0.001694183 | −24.88718926 | Hypo-methylated | NEPC\|CRPC-Adeno DMRs |
| chr7 | 101139439 | 101144324 | 9.81E−05 | −22.89097744 | Hypo-methylated | NEPC\|CRPC-Adeno DMRs |
| chr7 | 101414527 | 101425168 | 0.004583371 | −24.94377231 | Hypo-methylated | NEPC\|CRPC-Adeno DMRs |
| chr7 | 101621974 | 101622208 | 5.17E−06 | −54.88245614 | Hypo-methylated | NEPC\|CRPC-Adeno DMRs |
| chr7 | 101665615 | 101665823 | 0.000176134 | 24.99539474 | Hyper-methylated | NEPC\|CRPC-Adeno DMRs |
| chr7 | 101910353 | 101911242 | 0.001012154 | −22.14854995 | Hypo-methylated | NEPC\|CRPC-Adeno DMRs |
| chr7 | 101936417 | 101936520 | 2.46E−05 | 24.73095777 | Hyper-methylated | NEPC\|CRPC-Adeno DMRs |
| chr7 | 105278903 | 105284007 | 0.001931256 | −35.47421428 | Hypo-methylated | NEPC\|CRPC-Adeno DMRs |
| chr7 | 112725862 | 112725921 | 0.001694183 | −22.27105263 | Hypo-methylated | NEPC\|CRPC-Adeno DMRs |
| chr7 | 113727429 | 113727696 | 5.76E−07 | 24.05758217 | Hyper-methylated | NEPC\|CRPC-Adeno DMRs |
| chr7 | 113778706 | 114075862 | 1.47E−07 | −30.30602738 | Hypo-methylated | NEPC\|CRPC-Adeno DMRs |
| chr7 | 114344049 | 114555011 | 0.000496559 | −31.121275 | Hypo-methylated | NEPC\|CRPC-Adeno DMRs |
| chr7 | 114598663 | 114732973 | 0.001549499 | −30.72328216 | Hypo-methylated | NEPC\|CRPC-Adeno DMRs |
| chr7 | 115992732 | 116099339 | 6.27E−05 | −45.54482486 | Hypo-methylated | NEPC\|CRPC-Adeno DMRs |
| chr7 | 116173667 | 116267129 | 0.004583371 | −26.34456797 | Hypo-methylated | NEPC\|CRPC-Adeno DMRs |
| chr7 | 121950271 | 121951056 | 2.47E−13 | 27.29424703 | Hyper-methylated | NEPC\|CRPC-Adeno DMRs |
| chr7 | 121955826 | 121957299 | 6.21E−17 | 34.03237506 | Hyper-methylated | NEPC\|CRPC-Adeno DMRs |
| chr7 | 127992373 | 128001120 | 1.30E−05 | −20.45230586 | Hypo-methylated | NEPC\|CRPC-Adeno DMRs |
| chr7 | 128550114 | 128550429 | 1.22E−05 | −22.19552549 | Hypo-methylated | NEPC\|CRPC-Adeno DMRs |
| chr7 | 128843277 | 128849313 | 4.27E−05 | −26.79529825 | Hypo-methylated | NEPC\|CRPC-Adeno DMRs |
| chr7 | 129142639 | 129142818 | 2.92E−07 | −21.86956014 | Hypo-methylated | NEPC\|CRPC-Adeno DMRs |
| chr7 | 129412806 | 129412948 | 0.000549237 | −24.23127611 | Hypo-methylated | NEPC\|CRPC-Adeno DMRs |
| chr7 | 129781317 | 129781690 | 3.68E−07 | −24.34286432 | Hypo-methylated | NEPC\|CRPC-Adeno DMRs |
| chr7 | 129882516 | 129913332 | 1.21E−07 | −32.37775757 | Hypo-methylated | NEPC\|CRPC-Adeno DMRs |
| chr7 | 136952873 | 137139501 | 8.20E−11 | −43.46206543 | Hypo-methylated | NEPC\|CRPC-Adeno DMRs |
| chr7 | 137197633 | 137254332 | 0.000286322 | −33.9960858 | Hypo-methylated | NEPC\|CRPC-Adeno DMRs |
| chr7 | 137311605 | 137525101 | 0.001345309 | −30.27186895 | Hypo-methylated | NEPC\|CRPC-Adeno DMRs |
| chr7 | 137938182 | 138042037 | 0.00022941 | −20.49874223 | Hypo-methylated | NEPC\|CRPC-Adeno DMRs |
| chr7 | 138522694 | 138522803 | 0.001694183 | −34.89473684 | Hypo-methylated | NEPC\|CRPC-Adeno DMRs |
| chr7 | 138655625 | 138661116 | 0.00485991 | −21.486132 | Hypo-methylated | NEPC\|CRPC-Adeno DMRs |
| chr7 | 139298972 | 139299083 | 0.003113626 | −30.5287852 | Hypo-methylated | NEPC\|CRPC-Adeno DMRs |
| chr7 | 142370686 | 142481353 | 0.009285355 | −21.80197368 | Hypo-methylated | NEPC\|CRPC-Adeno DMRs |
| chr7 | 142708360 | 142906541 | 4.87E−08 | −31.26127606 | Hypo-methylated | NEPC\|CRPC-Adeno DMRs |
| chr7 | 143612178 | 143726580 | 5.01E−05 | −24.37532925 | Hypo-methylated | NEPC\|CRPC-Adeno DMRs |
| chr7 | 144098227 | 144098304 | 0.001694183 | −31.41666667 | Hypo-methylated | NEPC\|CRPC-Adeno DMRs |
| chr7 | 144098374 | 144098634 | 0.004750844 | −32.26220313 | Hypo-methylated | NEPC\|CRPC-Adeno DMRs |
| chr7 | 145058020 | 145058080 | 0.000246327 | 21.84517544 | Hyper-methylated | NEPC\|CRPC-Adeno DMRs |
| chr7 | 147960685 | 147979078 | 0.003525992 | 25.82192982 | Hyper-methylated | NEPC\|CRPC-Adeno DMRs |
| chr7 | 148984433 | 148984720 | 0.00222033 | 20.85131579 | Hyper-methylated | NEPC\|CRPC-Adeno DMRs |
| chr7 | 149095537 | 149095801 | 0.001036804 | −21.1622807 | Hypo-methylated | NEPC\|CRPC-Adeno DMRs |
| chr7 | 149471151 | 149471556 | 0.000359641 | −22.15748194 | Hypo-methylated | NEPC\|CRPC-Adeno DMRs |
| chr7 | 149524893 | 149525284 | 4.65E−07 | −39.64003599 | Hypo-methylated | NEPC\|CRPC-Adeno DMRs |
| chr7 | 150079771 | 150081431 | 5.99E−06 | −39.78262322 | Hypo-methylated | NEPC\|CRPC-Adeno DMRs |
| chr7 | 150621610 | 150621785 | 1.70E−05 | 29.44458204 | Hyper-methylated | NEPC\|CRPC-Adeno DMRs |
| chr7 | 150644519 | 150646261 | 1.42E−05 | 25.27178943 | Hyper-methylated | NEPC\|CRPC-Adeno DMRs |
| chr7 | 150662422 | 150662622 | 5.50E−05 | −23.34405458 | Hypo-methylated | NEPC\|CRPC-Adeno DMRs |
| chr7 | 150667475 | 150671111 | 1.30E−06 | −43.13443251 | Hypo-methylated | NEPC\|CRPC-Adeno DMRs |
| chr7 | 150675719 | 150676606 | 5.93E−07 | −20.48260383 | Hypo-methylated | NEPC\|CRPC-Adeno DMRs |
| chr7 | 150758411 | 150760530 | 1.43E−05 | 34.85106115 | Hyper-methylated | NEPC\|CRPC-Adeno DMRs |
| chr7 | 150808939 | 150813925 | 8.18E−54 | 44.44418402 | Hyper-methylated | NEPC\|CRPC-Adeno DMRs |
| chr7 | 150893625 | 150900164 | 3.37E−05 | −25.05822328 | Hypo-methylated | NEPC\|CRPC-Adeno DMRs |
| chr7 | 151092893 | 151093764 | 6.06E−10 | −22.94295504 | Hypo-methylated | NEPC\|CRPC-Adeno DMRs |
| chr7 | 151106286 | 151106600 | 2.74E−09 | −20.15992325 | Hypo-methylated | NEPC\|CRPC-Adeno DMRs |
| chr7 | 151393319 | 151394486 | 4.14E−08 | −23.24630848 | Hypo-methylated | NEPC\|CRPC-Adeno DMRs |
| chr7 | 151429904 | 151433341 | 0.00011018 | −34.74701754 | Hypo-methylated | NEPC\|CRPC-Adeno DMRs |
| chr7 | 152264791 | 152264976 | 0.000549237 | −25.62052632 | Hypo-methylated | NEPC\|CRPC-Adeno DMRs |
| chr7 | 152312238 | 152312414 | 0.001694183 | −32.59210526 | Hypo-methylated | NEPC\|CRPC-Adeno DMRs |
| chr7 | 152622035 | 152623255 | 1.86E−28 | 25.71285637 | Hyper-methylated | NEPC\|CRPC-Adeno DMRs |
| chr7 | 153446785 | 153549327 | 5.50E−05 | −28.3682223 | Hypo-methylated | NEPC\|CRPC-Adeno DMRs |
| chr7 | 153583498 | 153585619 | 6.56E−12 | 20.91698318 | Hyper-methylated | NEPC\|CRPC-Adeno DMRs |
| chr7 | 153748243 | 153749230 | 1.27E−05 | 31.65437859 | Hyper-methylated | NEPC\|CRPC-Adeno DMRs |
| chr7 | 153749311 | 153750276 | 6.59E−08 | 24.22265676 | Hyper-methylated | NEPC\|CRPC-Adeno DMRs |
| chr7 | 154001966 | 154002060 | 0.000477706 | 22.34518519 | Hyper-methylated | NEPC\|CRPC-Adeno DMRs |
| chr7 | 154679155 | 154684455 | 1.94E−06 | −22.44733109 | Hypo-methylated | NEPC\|CRPC-Adeno DMRs |
| chr7 | 154700060 | 154701586 | 0.005403657 | −23.3014295 | Hypo-methylated | NEPC\|CRPC-Adeno DMRs |
| chr7 | 154961046 | 154961253 | 0.001694183 | −26.43859649 | Hypo-methylated | NEPC\|CRPC-Adeno DMRs |
| chr7 | 155165258 | 155168194 | 2.90E−27 | 24.26971502 | Hyper-methylated | NEPC\|CRPC-Adeno DMRs |
| chr7 | 155175138 | 155175350 | 0.002094266 | 23.82149123 | Hyper-methylated | NEPC\|CRPC-Adeno DMRs |
| chr7 | 155249625 | 155250533 | 2.81E−34 | 20.24216956 | Hyper-methylated | NEPC\|CRPC-Adeno DMRs |
| chr7 | 155290337 | 155302510 | 5.20E−06 | 20.89056261 | Hyper-methylated | NEPC\|CRPC-Adeno DMRs |
| chr7 | 155588470 | 155588837 | 0.001545937 | −26.29805068 | Hypo-methylated | NEPC\|CRPC-Adeno DMRs |
| chr7 | 155638043 | 155663408 | 2.24E−06 | −22.9206979 | Hypo-methylated | NEPC\|CRPC-Adeno DMRs |
| chr7 | 155714959 | 155747399 | 3.86E−09 | −23.07660444 | Hypo-methylated | NEPC\|CRPC-Adeno DMRs |
| chr7 | 155794863 | 155832993 | 1.55E−10 | −22.35542129 | Hypo-methylated | NEPC\|CRPC-Adeno DMRs |
| chr7 | 155833027 | 155836135 | 9.75E−08 | −28.87053472 | Hypo-methylated | NEPC\|CRPC-Adeno DMRs |
| chr7 | 155836603 | 155867869 | 2.57E−11 | −23.74940534 | Hypo-methylated | NEPC\|CRPC-Adeno DMRs |
| chr7 | 155868074 | 155919738 | 7.69E−20 | −25.40228613 | Hypo-methylated | NEPC\|CRPC-Adeno DMRs |
| chr7 | 155920431 | 155922570 | 0.002976353 | −25.76846528 | Hypo-methylated | NEPC\|CRPC-Adeno DMRs |
| chr7 | 155935971 | 155939248 | 0.000601653 | −23.24230306 | Hypo-methylated | NEPC\|CRPC-Adeno DMRs |
| chr7 | 155982087 | 156029669 | 1.42E−13 | −28.43968047 | Hypo-methylated | NEPC\|CRPC-Adeno DMRs |
| chr7 | 157348105 | 157350761 | 4.24E−06 | 25.56256498 | Hyper-methylated | NEPC\|CRPC-Adeno DMRs |
| chr7 | 157403114 | 157403257 | 0.004750844 | 22.56158626 | Hyper-methylated | NEPC\|CRPC-Adeno DMRs |
| chr7 | 157423165 | 157423277 | 0.000549237 | 53.27493734 | Hyper-methylated | NEPC\|CRPC-Adeno DMRs |
| chr7 | 157454381 | 157455220 | 7.40E−05 | 29.11805068 | Hyper-methylated | NEPC\|CRPC-Adeno DMRs |
| chr7 | 157623228 | 157623283 | 1.70E−05 | −50.66327485 | Hypo-methylated | NEPC\|CRPC-Adeno DMRs |
| chr7 | 157690139 | 157690236 | 5.50E−05 | −34.76632554 | Hypo-methylated | NEPC\|CRPC-Adeno DMRs |
| chr7 | 157808922 | 157809023 | 0.000549237 | −24.04135338 | Hypo-methylated | NEPC\|CRPC-Adeno DMRs |
| chr7 | 157818540 | 157818816 | 9.59E−05 | −26.57935538 | Hypo-methylated | NEPC\|CRPC-Adeno DMRs |
| chr7 | 158207219 | 158207489 | 1.70E−05 | −33.48434211 | Hypo-methylated | NEPC\|CRPC-Adeno DMRs |
| chr7 | 158240210 | 158240447 | 1.55E−06 | −36.61773879 | Hypo-methylated | NEPC\|CRPC-Adeno DMRs |
| chr7 | 158749927 | 158751134 | 3.60E−08 | −23.18690574 | Hypo-methylated | NEPC\|CRPC-Adeno DMRs |
| chr7 | 158765163 | 158766524 | 8.65E−16 | 31.75989633 | Hyper-methylated | NEPC\|CRPC-Adeno DMRs |
| chr7 | 158823239 | 158824137 | 3.75E−05 | −23.994282 | Hypo-methylated | NEPC\|CRPC-Adeno DMRs |
| chr7 | 158828603 | 158828645 | 0.001694183 | 30.85602241 | Hyper-methylated | NEPC\|CRPC-Adeno DMRs |
| chr7 | 158933047 | 158935637 | 0.004222625 | 20.15096716 | Hyper-methylated | NEPC\|CRPC-Adeno DMRs |
| chr8 | 548319 | 554437 | 0.009458442 | −35.44691488 | Hypo-methylated | NEPC\|CRPC-Adeno DMRs |
| chr8 | 563734 | 576584 | 0.000549237 | −40.41298186 | Hypo-methylated | NEPC\|CRPC-Adeno DMRs |
| chr8 | 687072 | 687434 | 3.40E−08 | 23.17620574 | Hyper-methylated | NEPC\|CRPC-Adeno DMRs |
| chr8 | 688020 | 688038 | 0.000176134 | 25.75520833 | Hyper-methylated | NEPC\|CRPC-Adeno DMRs |
| chr8 | 688754 | 689448 | 2.90E−13 | −22.82425701 | Hypo-methylated | NEPC\|CRPC-Adeno DMRs |
| chr8 | 933960 | 936280 | 0.000549237 | −24.40636255 | Hypo-methylated | NEPC\|CRPC-Adeno DMRs |
| chr8 | 950333 | 962617 | 0.000342136 | −27.82898801 | Hypo-methylated | NEPC\|CRPC-Adeno DMRs |
| chr8 | 1019486 | 1041203 | 0.003095591 | −25.56203704 | Hypo-methylated | NEPC\|CRPC-Adeno DMRs |
| chr8 | 1054052 | 1105355 | 2.46E−12 | −26.32933308 | Hypo-methylated | NEPC\|CRPC-Adeno DMRs |
| chr8 | 1128147 | 1137662 | 8.33E−06 | −26.51896592 | Hypo-methylated | NEPC\|CRPC-Adeno DMRs |
| chr8 | 1143735 | 1154350 | 0.000181909 | −28.4736728 | Hypo-methylated | NEPC\|CRPC-Adeno DMRs |
| chr8 | 1205069 | 1284153 | 7.88E−10 | −23.19659827 | Hypo-methylated | NEPC\|CRPC-Adeno DMRs |
| chr8 | 1287319 | 1307512 | 6.31E−05 | −28.80687831 | Hypo-methylated | NEPC\|CRPC-Adeno DMRs |
| chr8 | 1312025 | 1315290 | 5.84E−05 | −23.65079365 | Hypo-methylated | NEPC\|CRPC-Adeno DMRs |
| chr8 | 1362644 | 1362784 | 0.004750844 | −26.07539683 | Hypo-methylated | NEPC\|CRPC-Adeno DMRs |
| chr8 | 1372805 | 1390829 | 4.74E−05 | −26.26034997 | Hypo-methylated | NEPC\|CRPC-Adeno DMRs |
| chr8 | 1414086 | 1414209 | 0.001012154 | −30.51247166 | Hypo-methylated | NEPC\|CRPC-Adeno DMRs |
| chr8 | 1444068 | 1449174 | 1.70E−05 | −28.34107143 | Hypo-methylated | NEPC\|CRPC-Adeno DMRs |
| chr8 | 1474621 | 1496348 | 0.000220189 | −29.76194367 | Hypo-methylated | NEPC\|CRPC-Adeno DMRs |
| chr8 | 1497354 | 1497492 | 0.000124569 | −20.29679233 | Hypo-methylated | NEPC\|CRPC-Adeno DMRs |
| chr8 | 1502748 | 1581080 | 2.42E−09 | −25.3213242 | Hypo-methylated | NEPC\|CRPC-Adeno DMRs |
| chr8 | 1585227 | 1611579 | 1.30E−09 | −25.05703655 | Hypo-methylated | NEPC\|CRPC-Adeno DMRs |
| chr8 | 1615531 | 1633331 | 0.00324027 | −22.63756614 | Hypo-methylated | NEPC\|CRPC-Adeno DMRs |
| chr8 | 1914991 | 1915048 | 0.003525992 | −49.48412698 | Hypo-methylated | NEPC\|CRPC-Adeno DMRs |
| chr8 | 1994504 | 1994586 | 0.001694183 | −31.07671958 | Hypo-methylated | NEPC\|CRPC-Adeno DMRs |
| chr8 | 4664909 | 4773245 | 1.82E−05 | −31.50027963 | Hypo-methylated | NEPC\|CRPC-Adeno DMRs |
| chr8 | 8699509 | 8699666 | 0.000176134 | 23.525 | Hyper-methylated | NEPC\|CRPC-Adeno DMRs |
| chr8 | 8702914 | 8703087 | 0.000549237 | −50.08253968 | Hypo-methylated | NEPC\|CRPC-Adeno DMRs |
| chr8 | 9755120 | 9756237 | 0.000154411 | 22.25699446 | Hyper-methylated | NEPC\|CRPC-Adeno DMRs |
| chr8 | 10260705 | 10262629 | 4.74E−05 | −37.12539683 | Hypo-methylated | NEPC\|CRPC-Adeno DMRs |
| chr8 | 10823215 | 10823356 | 0.000176134 | −50.19305556 | Hypo-methylated | NEPC\|CRPC-Adeno DMRs |
| chr8 | 10897150 | 10906939 | 1.13E−06 | −28.26510076 | Hypo-methylated | NEPC\|CRPC-Adeno DMRs |
| chr8 | 10950065 | 10950227 | 0.000176134 | 39.10277778 | Hyper-methylated | NEPC\|CRPC-Adeno DMRs |
| chr8 | 11318849 | 11319758 | 0.001802623 | −32.13730159 | Hypo-methylated | NEPC\|CRPC-Adeno DMRs |
| chr8 | 11444763 | 11457098 | 1.24E−05 | −24.16021424 | Hypo-methylated | NEPC\|CRPC-Adeno DMRs |
| chr8 | 11558908 | 11559680 | 5.17E−06 | −28.58585859 | Hypo-methylated | NEPC\|CRPC-Adeno DMRs |
| chr8 | 11565860 | 11566334 | 1.51E−13 | 28.37685849 | Hyper-methylated | NEPC\|CRPC-Adeno DMRs |
| chr8 | 11566725 | 11566774 | 0.004750844 | 20.8968254 | Hyper-methylated | NEPC\|CRPC-Adeno DMRs |
| chr8 | 11571961 | 11583647 | 0.003327791 | −24.87320261 | Hypo-methylated | NEPC\|CRPC-Adeno DMRs |
| chr8 | 11597630 | 11597787 | 0.000549237 | −33.01071429 | Hypo-methylated | NEPC\|CRPC-Adeno DMRs |
| chr8 | 11764122 | 11793605 | 0.000377187 | −22.31187771 | Hypo-methylated | NEPC\|CRPC-Adeno DMRs |
| chr8 | 12990473 | 12991207 | 3.18E−05 | 21.23221748 | Hyper-methylated | NEPC\|CRPC-Adeno DMRs |
| chr8 | 13001419 | 13099347 | 6.14E−12 | −43.75187325 | Hypo-methylated | NEPC\|CRPC-Adeno DMRs |
| chr8 | 13424251 | 13424290 | 0.003113626 | 26.33333333 | Hyper-methylated | NEPC\|CRPC-Adeno DMRs |
| chr8 | 15094264 | 15094372 | 0.002692265 | 20.30185185 | Hyper-methylated | NEPC\|CRPC-Adeno DMRs |
| chr8 | 20170522 | 20170593 | 0.001694183 | −37.83194444 | Hypo-methylated | NEPC\|CRPC-Adeno DMRs |
| chr8 | 21503796 | 21503979 | 0.000953892 | −35.78457925 | Hypo-methylated | NEPC\|CRPC-Adeno DMRs |
| chr8 | 21647244 | 21647747 | 2.53E−14 | 24.90639297 | Hyper-methylated | NEPC\|CRPC-Adeno DMRs |
| chr8 | 21974245 | 21974541 | 6.91E−08 | −20.11501736 | Hypo-methylated | NEPC\|CRPC-Adeno DMRs |
| chr8 | 22004991 | 22005216 | 0.001202962 | −21.21948335 | Hypo-methylated | NEPC\|CRPC-Adeno DMRs |
| chr8 | 22411622 | 22411875 | 0.005574264 | −23.02929717 | Hypo-methylated | NEPC\|CRPC-Adeno DMRs |
| chr8 | 22595457 | 22595520 | 0.000549237 | −27.67460317 | Hypo-methylated | NEPC\|CRPC-Adeno DMRs |
| chr8 | 22872097 | 22877339 | 0.00067511 | −21.37640523 | Hypo-methylated | NEPC\|CRPC-Adeno DMRs |
| chr8 | 26558310 | 26721552 | 0.000975454 | −33.78126653 | Hypo-methylated | NEPC\|CRPC-Adeno DMRs |
| chr8 | 26721749 | 26721984 | 1.13E−06 | −22.59403935 | Hypo-methylated | NEPC\|CRPC-Adeno DMRs |
| chr8 | 26723523 | 26723602 | 2.46E−05 | 23.41193529 | Hyper-methylated | NEPC\|CRPC-Adeno DMRs |
| chr8 | 26724441 | 26724503 | 7.37E−05 | 21.10555556 | Hyper-methylated | NEPC\|CRPC-Adeno DMRs |
| chr8 | 28111255 | 28143729 | 0.000224358 | −23.8884127 | Hypo-methylated | NEPC\|CRPC-Adeno DMRs |
| chr8 | 28205271 | 28223052 | 0.000189694 | 26.08636204 | Hyper-methylated | NEPC\|CRPC-Adeno DMRs |
| chr8 | 29870043 | 29870110 | 0.001694183 | −48.66666667 | Hypo-methylated | NEPC\|CRPC-Adeno DMRs |
| chr8 | 33371946 | 33372001 | 4.92E−05 | −30.20517429 | Hypo-methylated | NEPC\|CRPC-Adeno DMRs |
| chr8 | 37655471 | 37655639 | 5.01E−05 | 24.75416667 | Hyper-methylated | NEPC\|CRPC-Adeno DMRs |
| chr8 | 37755408 | 37755470 | 0.001012154 | −29.93061224 | Hypo-methylated | NEPC\|CRPC-Adeno DMRs |
| chr8 | 37822530 | 37822575 | 1.70E−05 | 22.78888889 | Hyper-methylated | NEPC\|CRPC-Adeno DMRs |
| chr8 | 38241133 | 38241275 | 0.003113626 | −37.36296296 | Hypo-methylated | NEPC\|CRPC-Adeno DMRs |
| chr8 | 41137893 | 41148108 | 0.000492768 | −29.14269905 | Hypo-methylated | NEPC\|CRPC-Adeno DMRs |
| chr8 | 41571702 | 41571800 | 0.001694183 | −42.48703704 | Hypo-methylated | NEPC\|CRPC-Adeno DMRs |
| chr8 | 42947103 | 42947166 | 0.000324528 | −34.04681373 | Hypo-methylated | NEPC\|CRPC-Adeno DMRs |
| chr8 | 43101980 | 43102561 | 4.95E−06 | 22.33819444 | Hyper-methylated | NEPC\|CRPC-Adeno DMRs |
| chr8 | 43132180 | 43132260 | 0.001694183 | −22.24222222 | Hypo-methylated | NEPC\|CRPC-Adeno DMRs |
| chr8 | 43147389 | 43252192 | 0.007832634 | −22.90622723 | Hypo-methylated | NEPC\|CRPC-Adeno DMRs |
| chr8 | 49018246 | 49230159 | 1.26E−12 | −26.03887634 | Hypo-methylated | NEPC\|CRPC-Adeno DMRs |
| chr8 | 49351073 | 49391726 | 3.27E−07 | −26.33167454 | Hypo-methylated | NEPC\|CRPC-Adeno DMRs |
| chr8 | 49628918 | 49675312 | 4.20E−05 | −21.32012783 | Hypo-methylated | NEPC\|CRPC-Adeno DMRs |
| chr8 | 49718100 | 49778204 | 5.16E−09 | −21.23006633 | Hypo-methylated | NEPC\|CRPC-Adeno DMRs |
| chr8 | 49783232 | 49783262 | 0.000549237 | 20.96031746 | Hyper-methylated | NEPC\|CRPC-Adeno DMRs |
| chr8 | 49783548 | 49826443 | 1.50E−07 | −23.0173992 | Hypo-methylated | NEPC\|CRPC-Adeno DMRs |
| chr8 | 51413791 | 51605941 | 0.000220189 | −27.89756074 | Hypo-methylated | NEPC\|CRPC-Adeno DMRs |
| chr8 | 52721859 | 52721941 | 0.000549237 | −24.19323308 | Hypo-methylated | NEPC\|CRPC-Adeno DMRs |
| chr8 | 53013436 | 53022287 | 8.52E−09 | −27.09267998 | Hypo-methylated | NEPC\|CRPC-Adeno DMRs |
| chr8 | 53851773 | 53851807 | 0.000324528 | 28.97828947 | Hyper-methylated | NEPC\|CRPC-Adeno DMRs |
| chr8 | 54395986 | 54397093 | 1.70E−05 | −29.07256653 | Hypo-methylated | NEPC\|CRPC-Adeno DMRs |
| chr8 | 55370662 | 55370710 | 0.000176134 | 25.98305921 | Hyper-methylated | NEPC\|CRPC-Adeno DMRs |
| chr8 | 55370797 | 55371035 | 4.28E−07 | 23.38431495 | Hyper-methylated | NEPC\|CRPC-Adeno DMRs |
| chr8 | 55372054 | 55372088 | 0.000101637 | 23.82931867 | Hyper-methylated | NEPC\|CRPC-Adeno DMRs |
| chr8 | 55372219 | 55372294 | 0.00217795 | 27.33363065 | Hyper-methylated | NEPC\|CRPC-Adeno DMRs |
| chr8 | 55378973 | 55379838 | 2.68E−23 | 29.38632166 | Hyper-methylated | NEPC\|CRPC-Adeno DMRs |
| chr8 | 55380107 | 55380186 | 4.74E−05 | 29.38860118 | Hyper-methylated | NEPC\|CRPC-Adeno DMRs |
| chr8 | 55382681 | 55383183 | 2.99E−15 | 24.6062471 | Hyper-methylated | NEPC\|CRPC-Adeno DMRs |
| chr8 | 56014105 | 56014265 | 7.53E−06 | 23.2364931 | Hyper-methylated | NEPC\|CRPC-Adeno DMRs |
| chr8 | 56015215 | 56015256 | 2.94E−06 | 21.6254085 | Hyper-methylated | NEPC\|CRPC-Adeno DMRs |
| chr8 | 57069739 | 57070014 | 6.33E−06 | 21.27477314 | Hyper-methylated | NEPC\|CRPC-Adeno DMRs |
| chr8 | 57107106 | 57111144 | 0.000348917 | −32.23990337 | Hypo-methylated | NEPC\|CRPC-Adeno DMRs |
| chr8 | 57358301 | 57358423 | 1.37E−06 | 22.77092732 | Hyper-methylated | NEPC\|CRPC-Adeno DMRs |
| chr8 | 57358663 | 57358739 | 7.89E−07 | 28.79678363 | Hyper-methylated | NEPC\|CRPC-Adeno DMRs |
| chr8 | 57359077 | 57359133 | 0.001883547 | 21.38636364 | Hyper-methylated | NEPC\|CRPC-Adeno DMRs |
| chr8 | 57359210 | 57359677 | 4.85E−07 | 25.97544034 | Hyper-methylated | NEPC\|CRPC-Adeno DMRs |
| chr8 | 58055301 | 58055505 | 3.03E−07 | 24.44324943 | Hyper-methylated | NEPC\|CRPC-Adeno DMRs |
| chr8 | 59138019 | 59208728 | 9.09E−05 | −21.76456299 | Hypo-methylated | NEPC\|CRPC-Adeno DMRs |
| chr8 | 59231078 | 59231132 | 0.001694183 | 27.86754386 | Hyper-methylated | NEPC\|CRPC-Adeno DMRs |
| chr8 | 61936439 | 61990489 | 0.000263522 | −34.99536842 | Hypo-methylated | NEPC\|CRPC-Adeno DMRs |
| chr8 | 63161560 | 63161698 | 8.08E−07 | 21.24536341 | Hyper-methylated | NEPC\|CRPC-Adeno DMRs |
| chr8 | 63781881 | 63895905 | 0.00222033 | −23.47716165 | Hypo-methylated | NEPC\|CRPC-Adeno DMRs |
| chr8 | 65281783 | 65282554 | 7.50E−07 | 21.76033835 | Hyper-methylated | NEPC\|CRPC-Adeno DMRs |
| chr8 | 65282680 | 65283663 | 5.22E−06 | 24.8296479 | Hyper-methylated | NEPC\|CRPC-Adeno DMRs |
| chr8 | 65490204 | 65492053 | 4.78E−07 | 20.79174339 | Hyper-methylated | NEPC\|CRPC-Adeno DMRs |
| chr8 | 66876254 | 66928541 | 0.000549237 | −21.41461988 | Hypo-methylated | NEPC\|CRPC-Adeno DMRs |
| chr8 | 67090538 | 67090588 | 0.000176134 | −23.46590557 | Hypo-methylated | NEPC\|CRPC-Adeno DMRs |
| chr8 | 68349486 | 68386618 | 5.17E−06 | −28.47749867 | Hypo-methylated | NEPC\|CRPC-Adeno DMRs |
| chr8 | 68391116 | 68713335 | 2.39E−06 | −25.58820244 | Hypo-methylated | NEPC\|CRPC-Adeno DMRs |
| chr8 | 68779781 | 68858398 | 5.36E−06 | −45.11140351 | Hypo-methylated | NEPC\|CRPC-Adeno DMRs |
| chr8 | 68877185 | 69205787 | 1.70E−08 | −39.25024889 | Hypo-methylated | NEPC\|CRPC-Adeno DMRs |
| chr8 | 72756051 | 72756111 | 6.91E−08 | 23.32697368 | Hyper-methylated | NEPC\|CRPC-Adeno DMRs |
| chr8 | 77595300 | 77832645 | 5.16E−07 | −21.93259846 | Hypo-methylated | NEPC\|CRPC-Adeno DMRs |
| chr8 | 78015287 | 79374462 | 1.12E−09 | −37.25030795 | Hypo-methylated | NEPC\|CRPC-Adeno DMRs |
| chr8 | 81213465 | 81213724 | 0.000200814 | −25.23315789 | Hypo-methylated | NEPC\|CRPC-Adeno DMRs |
| chr8 | 86440217 | 86569170 | 0.000148945 | −34.42812714 | Hypo-methylated | NEPC\|CRPC-Adeno DMRs |
| chr8 | 87094225 | 87354341 | 4.14E−05 | −26.55019952 | Hypo-methylated | NEPC\|CRPC-Adeno DMRs |
| chr8 | 96186989 | 96187034 | 0.001694183 | −61.18140838 | Hypo-methylated | NEPC\|CRPC-Adeno DMRs |
| chr8 | 96835985 | 96957807 | 3.79E−05 | −30.94064536 | Hypo-methylated | NEPC\|CRPC-Adeno DMRs |
| chr8 | 101479899 | 101506734 | 0.003327791 | 24.25134589 | Hyper-methylated | NEPC\|CRPC-Adeno DMRs |
| chr8 | 104117790 | 104118252 | 0.000549237 | −45.44586466 | Hypo-methylated | NEPC\|CRPC-Adeno DMRs |
| chr8 | 105645352 | 105645419 | 0.003113626 | −29.70921053 | Hypo-methylated | NEPC\|CRPC-Adeno DMRs |
| chr8 | 107600340 | 107637181 | 0.009458442 | −36.5162768 | Hypo-methylated | NEPC\|CRPC-Adeno DMRs |
| chr8 | 110986149 | 110986669 | 0.001255081 | 23.20994152 | Hyper-methylated | NEPC\|CRPC-Adeno DMRs |
| chr8 | 116382702 | 116448558 | 0.000975454 | −20.41077694 | Hypo-methylated | NEPC\|CRPC-Adeno DMRs |
| chr8 | 116679744 | 116679835 | 5.01E−05 | 34.79692982 | Hyper-methylated | NEPC\|CRPC-Adeno DMRs |
| chr8 | 117172975 | 117300476 | 0.000581135 | −20.66118421 | Hypo-methylated | NEPC\|CRPC-Adeno DMRs |
| chr8 | 119122182 | 119123162 | 9.74E−06 | −20.65010121 | Hypo-methylated | NEPC\|CRPC-Adeno DMRs |
| chr8 | 119270296 | 119299844 | 0.000484821 | 20.40695489 | Hyper-methylated | NEPC\|CRPC-Adeno DMRs |
| chr8 | 119346995 | 119630970 | 5.83E−06 | 20.80061505 | Hyper-methylated | NEPC\|CRPC-Adeno DMRs |
| chr8 | 121206379 | 121357766 | 0.001228429 | −25.82458751 | Hypo-methylated | NEPC\|CRPC-Adeno DMRs |
| chr8 | 121567734 | 121822891 | 0.000164003 | −27.44174663 | Hypo-methylated | NEPC\|CRPC-Adeno DMRs |
| chr8 | 121878018 | 122180408 | 6.80E−07 | −32.68393901 | Hypo-methylated | NEPC\|CRPC-Adeno DMRs |
| chr8 | 123616475 | 123655595 | 0.001549499 | −20.33708882 | Hypo-methylated | NEPC\|CRPC-Adeno DMRs |
| chr8 | 128681344 | 128704665 | 0.005239183 | −28.59641929 | Hypo-methylated | NEPC\|CRPC-Adeno DMRs |
| chr8 | 131033464 | 131033693 | 0.000303685 | 28.92748538 | Hyper-methylated | NEPC\|CRPC-Adeno DMRs |
| chr8 | 131964088 | 132051742 | 2.07E−05 | −24.30661376 | Hypo-methylated | NEPC\|CRPC-Adeno DMRs |
| chr8 | 132053079 | 132054680 | 2.44E−07 | 21.25462751 | Hyper-methylated | NEPC\|CRPC-Adeno DMRs |
| chr8 | 134334295 | 134390777 | 6.45E−06 | 23.48293074 | Hyper-methylated | NEPC\|CRPC-Adeno DMRs |
| chr8 | 135728694 | 135734663 | 0.004750844 | −33.27154605 | Hypo-methylated | NEPC\|CRPC-Adeno DMRs |
| chr8 | 140714605 | 140716218 | 2.74E−37 | −24.01956976 | Hypo-methylated | NEPC\|CRPC-Adeno DMRs |
| chr8 | 140818431 | 140818586 | 0.001545937 | −43.47343577 | Hypo-methylated | NEPC\|CRPC-Adeno DMRs |
| chr8 | 140854221 | 140855177 | 0.004750844 | −21.53007519 | Hypo-methylated | NEPC\|CRPC-Adeno DMRs |
| chr8 | 140859734 | 140860240 | 3.12E−07 | −39.04258328 | Hypo-methylated | NEPC\|CRPC-Adeno DMRs |
| chr8 | 140888175 | 140893308 | 1.55E−06 | −38.25438596 | Hypo-methylated | NEPC\|CRPC-Adeno DMRs |
| chr8 | 140929804 | 140931477 | 1.37E−06 | −41.25035505 | Hypo-methylated | NEPC\|CRPC-Adeno DMRs |
| chr8 | 141057225 | 141057521 | 7.87E−11 | −41.89473684 | Hypo-methylated | NEPC\|CRPC-Adeno DMRs |
| chr8 | 141172065 | 141208733 | 0.001600753 | 20.31571207 | Hyper-methylated | NEPC\|CRPC-Adeno DMRs |
| chr8 | 141347658 | 141347710 | 0.000176134 | −47.75352444 | Hypo-methylated | NEPC\|CRPC-Adeno DMRs |
| chr8 | 142157126 | 142157247 | 1.70E−05 | 39.78210526 | Hyper-methylated | NEPC\|CRPC-Adeno DMRs |
| chr8 | 142187405 | 142189600 | 0.002432731 | −25.09005848 | Hypo-methylated | NEPC\|CRPC-Adeno DMRs |
| chr8 | 142230701 | 142231546 | 4.59E−06 | 29.44081384 | Hyper-methylated | NEPC\|CRPC-Adeno DMRs |
| chr8 | 142287046 | 142287358 | 6.50E−07 | −36.90443213 | Hypo-methylated | NEPC\|CRPC-Adeno DMRs |
| chr8 | 142292238 | 142292320 | 0.001694183 | −39.36754386 | Hypo-methylated | NEPC\|CRPC-Adeno DMRs |
| chr8 | 142378082 | 142380311 | 9.82E−05 | −30.31659919 | Hypo-methylated | NEPC\|CRPC-Adeno DMRs |
| chr8 | 142729064 | 142733052 | 0.002174767 | 22.99073513 | Hyper-methylated | NEPC\|CRPC-Adeno DMRs |
| chr8 | 142735997 | 142736179 | 0.002976353 | 30.04285714 | Hyper-methylated | NEPC\|CRPC-Adeno DMRs |
| chr8 | 142857802 | 142858608 | 0.003113626 | −30.82116228 | Hypo-methylated | NEPC\|CRPC-Adeno DMRs |
| chr8 | 142986649 | 142986787 | 0.001694183 | −27.68630604 | Hypo-methylated | NEPC\|CRPC-Adeno DMRs |
| chr8 | 143070468 | 143073102 | 0.003977693 | 21.28416179 | Hyper-methylated | NEPC\|CRPC-Adeno DMRs |
| chr8 | 143227736 | 143227970 | 1.78E−05 | 28.73136671 | Hyper-methylated | NEPC\|CRPC-Adeno DMRs |
| chr8 | 143237761 | 143240116 | 0.000324528 | 22.55460526 | Hyper-methylated | NEPC\|CRPC-Adeno DMRs |
| chr8 | 143375846 | 143376371 | 1.94E−06 | −22.97604428 | Hypo-methylated | NEPC\|CRPC-Adeno DMRs |
| chr8 | 143395943 | 143396417 | 0.000101637 | 36.03625731 | Hyper-methylated | NEPC\|CRPC-Adeno DMRs |
| chr8 | 143420067 | 143424381 | 3.82E−06 | −27.72003533 | Hypo-methylated | NEPC\|CRPC-Adeno DMRs |
| chr8 | 143574852 | 143574995 | 0.001694183 | −23.15789474 | Hypo-methylated | NEPC\|CRPC-Adeno DMRs |
| chr8 | 143611978 | 143612251 | 5.36E−06 | −30.31071603 | Hypo-methylated | NEPC\|CRPC-Adeno DMRs |
| chr8 | 143776517 | 143781748 | 1.86E−06 | −29.86809211 | Hypo-methylated | NEPC\|CRPC-Adeno DMRs |
| chr8 | 143854034 | 143854886 | 0.000124569 | −27.48467697 | Hypo-methylated | NEPC\|CRPC-Adeno DMRs |
| chr8 | 144153728 | 144155326 | 5.08E−07 | −33.6406981 | Hypo-methylated | NEPC\|CRPC-Adeno DMRs |
| chr8 | 144269928 | 144270805 | 7.47E−05 | −21.38011696 | Hypo-methylated | NEPC\|CRPC-Adeno DMRs |
| chr8 | 144551891 | 144554130 | 5.68E−05 | −22.36310983 | Hypo-methylated | NEPC\|CRPC-Adeno DMRs |
| chr8 | 144572979 | 144573731 | 0.000348917 | −23.12834725 | Hypo-methylated | NEPC\|CRPC-Adeno DMRs |
| chr8 | 144597094 | 144598983 | 0.005899637 | −26.05497076 | Hypo-methylated | NEPC\|CRPC-Adeno DMRs |
| chr8 | 144640310 | 144640858 | 3.40E−14 | −25.97145642 | Hypo-methylated | NEPC\|CRPC-Adeno DMRs |
| chr8 | 144676049 | 144676152 | 0.000549237 | −35.33333333 | Hypo-methylated | NEPC\|CRPC-Adeno DMRs |
| chr8 | 144872811 | 144872958 | 7.58E−08 | −33.68888889 | Hypo-methylated | NEPC\|CRPC-Adeno DMRs |
| chr8 | 144949825 | 144951803 | 4.65E−07 | 24.157714 | Hyper-methylated | NEPC\|CRPC-Adeno DMRs |
| chr8 | 145023500 | 145025047 | 2.20E−11 | 20.34282365 | Hyper-methylated | NEPC\|CRPC-Adeno DMRs |
| chr8 | 145028177 | 145028481 | 0.00053414 | 21.5689333 | Hyper-methylated | NEPC\|CRPC-Adeno DMRs |
| chr8 | 145045257 | 145046910 | 0.00222033 | 26.45504386 | Hyper-methylated | NEPC\|CRPC-Adeno DMRs |
| chr8 | 145123622 | 145125045 | 0.007249297 | −20.62464146 | Hypo-methylated | NEPC\|CRPC-Adeno DMRs |
| chr8 | 145559841 | 145560204 | 0.002362173 | −20.19173629 | Hypo-methylated | NEPC\|CRPC-Adeno DMRs |
| chr8 | 145725616 | 145728631 | 3.12E−14 | 34.06405456 | Hyper-methylated | NEPC\|CRPC-Adeno DMRs |
| chr8 | 145758493 | 145758729 | 0.000231593 | −29.23278061 | Hypo-methylated | NEPC\|CRPC-Adeno DMRs |
| chr8 | 145804895 | 145805123 | 0.000549237 | 36.83333333 | Hyper-methylated | NEPC\|CRPC-Adeno DMRs |
| chr8 | 145871451 | 145871563 | 0.001545937 | 20.8414903 | Hyper-methylated | NEPC\|CRPC-Adeno DMRs |
| chr8 | 145987889 | 145988113 | 5.50E−05 | −44.52345679 | Hypo-methylated | NEPC\|CRPC-Adeno DMRs |
| chr9 | 235788 | 439410 | 3.93E−07 | −22.8038697 | Hypo-methylated | NEPC\|CRPC-Adeno DMRs |
| chr9 | 441358 | 441424 | 0.001694183 | −23.83564815 | Hypo-methylated | NEPC\|CRPC-Adeno DMRs |
| chr9 | 842531 | 842781 | 6.99E−06 | 20.17777778 | Hyper-methylated | NEPC\|CRPC-Adeno DMRs |
| chr9 | 842800 | 949018 | 0.002592833 | −32.05982906 | Hypo-methylated | NEPC\|CRPC-Adeno DMRs |
| chr9 | 971454 | 972033 | 3.03E−05 | 29.51770452 | Hyper-methylated | NEPC\|CRPC-Adeno DMRs |
| chr9 | 1509658 | 1637477 | 5.50E−05 | −35.56463326 | Hypo-methylated | NEPC\|CRPC-Adeno DMRs |
| chr9 | 1667754 | 2009700 | 9.61E−06 | −39.61967893 | Hypo-methylated | NEPC\|CRPC-Adeno DMRs |
| chr9 | 6716079 | 6717227 | 0.000275281 | −24.29486656 | Hypo-methylated | NEPC\|CRPC-Adeno DMRs |
| chr9 | 10285888 | 10583640 | 1.01E−08 | −27.56288032 | Hypo-methylated | NEPC\|CRPC-Adeno DMRs |
| chr9 | 21402547 | 21559374 | 1.00E−05 | 23.75232526 | Hyper-methylated | NEPC\|CRPC-Adeno DMRs |
| chr9 | 33750285 | 33750553 | 0.003090185 | −23.41535088 | Hypo-methylated | NEPC\|CRPC-Adeno DMRs |
| chr9 | 34624621 | 34624651 | 0.003327791 | 29.4786152 | Hyper-methylated | NEPC\|CRPC-Adeno DMRs |
| chr9 | 35791556 | 35791983 | 2.77E−09 | 31.25503289 | Hyper-methylated | NEPC\|CRPC-Adeno DMRs |
| chr9 | 36868247 | 36868435 | 0.000732983 | −34.24880383 | Hypo-methylated | NEPC\|CRPC-Adeno DMRs |
| chr9 | 37959915 | 37963171 | 0.000184724 | −21.5122807 | Hypo-methylated | NEPC\|CRPC-Adeno DMRs |
| chr9 | 38687298 | 38687897 | 1.42E−08 | −37.80715927 | Hypo-methylated | NEPC\|CRPC-Adeno DMRs |
| chr9 | 44178017 | 44219146 | 9.42E−05 | −25.81673547 | Hypo-methylated | NEPC\|CRPC-Adeno DMRs |
| chr9 | 44882443 | 45119196 | 1.48E−07 | −34.12267738 | Hypo-methylated | NEPC\|CRPC-Adeno DMRs |
| chr9 | 66837865 | 68291949 | 8.84E−05 | −24.46116765 | Hypo-methylated | NEPC\|CRPC-Adeno DMRs |
| chr9 | 68370862 | 68408947 | 0.000111341 | −25.01299799 | Hypo-methylated | NEPC\|CRPC-Adeno DMRs |
| chr9 | 68409254 | 68410815 | 0.000312629 | −20.63678191 | Hypo-methylated | NEPC\|CRPC-Adeno DMRs |
| chr9 | 71835666 | 71835938 | 1.55E−06 | −31.76680058 | Hypo-methylated | NEPC\|CRPC-Adeno DMRs |
| chr9 | 72027151 | 72027367 | 0.00678631 | 21.88581302 | Hyper-methylated | NEPC\|CRPC-Adeno DMRs |
| chr9 | 73002759 | 73002844 | 0.000832692 | −30.6125731 | Hypo-methylated | NEPC\|CRPC-Adeno DMRs |
| chr9 | 78694263 | 78739587 | 2.46E−05 | −25.44504644 | Hypo-methylated | NEPC\|CRPC-Adeno DMRs |
| chr9 | 78803362 | 78820992 | 0.000488426 | −24.02002064 | Hypo-methylated | NEPC\|CRPC-Adeno DMRs |
| chr9 | 80833961 | 80834093 | 0.003327791 | −21.36907895 | Hypo-methylated | NEPC\|CRPC-Adeno DMRs |
| chr9 | 89574420 | 89626722 | 1.76E−05 | −26.93626714 | Hypo-methylated | NEPC\|CRPC-Adeno DMRs |
| chr9 | 89773622 | 89920198 | 2.37E−08 | −33.99632622 | Hypo-methylated | NEPC\|CRPC-Adeno DMRs |
| chr9 | 90498352 | 90559236 | 3.54E−05 | −24.76810864 | Hypo-methylated | NEPC\|CRPC-Adeno DMRs |
| chr9 | 90626773 | 90682265 | 0.000337166 | −21.52256803 | Hypo-methylated | NEPC\|CRPC-Adeno DMRs |
| chr9 | 90890419 | 90976563 | 5.89E−05 | −20.14055777 | Hypo-methylated | NEPC\|CRPC-Adeno DMRs |
| chr9 | 91321416 | 91321931 | 1.33E−07 | −46.08765951 | Hypo-methylated | NEPC\|CRPC-Adeno DMRs |
| chr9 | 92299887 | 92300948 | 0.003808143 | −24.82050812 | Hypo-methylated | NEPC\|CRPC-Adeno DMRs |
| chr9 | 92858175 | 92860846 | 0.000392904 | −38.6157856 | Hypo-methylated | NEPC\|CRPC-Adeno DMRs |
| chr9 | 93955387 | 93956209 | 3.70E−11 | −35.12479821 | Hypo-methylated | NEPC\|CRPC-Adeno DMRs |
| chr9 | 96592222 | 96592301 | 0.001694183 | −27.4214425 | Hypo-methylated | NEPC\|CRPC-Adeno DMRs |
| chr9 | 97022286 | 97022355 | 4.74E−05 | 22.74510234 | Hyper-methylated | NEPC\|CRPC-Adeno DMRs |
| chr9 | 97837065 | 97839657 | 0.006847068 | −29.29022556 | Hypo-methylated | NEPC\|CRPC-Adeno DMRs |
| chr9 | 98032567 | 98032640 | 0.000549237 | −41.61353383 | Hypo-methylated | NEPC\|CRPC-Adeno DMRs |
| chr9 | 98256445 | 98257335 | 0.000340762 | −36.66137009 | Hypo-methylated | NEPC\|CRPC-Adeno DMRs |
| chr9 | 98527342 | 98527426 | 0.000549237 | −31.04511278 | Hypo-methylated | NEPC\|CRPC-Adeno DMRs |
| chr9 | 100593582 | 100593928 | 0.001883547 | −24.11961722 | Hypo-methylated | NEPC\|CRPC-Adeno DMRs |
| chr9 | 100617008 | 100620147 | 5.54E−17 | 23.37620992 | Hyper-methylated | NEPC\|CRPC-Adeno DMRs |
| chr9 | 102021680 | 102022088 | 1.77E−05 | −22.39809863 | Hypo-methylated | NEPC\|CRPC-Adeno DMRs |
| chr9 | 110350021 | 110350209 | 1.37E−07 | −20.70466792 | Hypo-methylated | NEPC\|CRPC-Adeno DMRs |
| chr9 | 111610649 | 111617412 | 2.94E−06 | −24.25595046 | Hypo-methylated | NEPC\|CRPC-Adeno DMRs |
| chr9 | 112705330 | 112705396 | 0.001694183 | 20.62614768 | Hyper-methylated | NEPC\|CRPC-Adeno DMRs |
| chr9 | 113801112 | 113801271 | 1.17E−08 | 33.14948078 | Hyper-methylated | NEPC\|CRPC-Adeno DMRs |
| chr9 | 115618930 | 115619278 | 0.002094266 | −31.81242922 | Hypo-methylated | NEPC\|CRPC-Adeno DMRs |
| chr9 | 116372198 | 116372423 | 0.001694183 | −52.99385965 | Hypo-methylated | NEPC\|CRPC-Adeno DMRs |
| chr9 | 116411417 | 116411676 | 0.000926684 | −50.38325163 | Hypo-methylated | NEPC\|CRPC-Adeno DMRs |
| chr9 | 117022084 | 117022330 | 0.000549237 | −39.03575606 | Hypo-methylated | NEPC\|CRPC-Adeno DMRs |
| chr9 | 124344198 | 124345027 | 0.002023529 | −26.45813434 | Hypo-methylated | NEPC\|CRPC-Adeno DMRs |
| chr9 | 124620028 | 124621382 | 0.001694183 | −56.61017157 | Hypo-methylated | NEPC\|CRPC-Adeno DMRs |
| chr9 | 124989640 | 124989856 | 3.68E−07 | −22.84333641 | Hypo-methylated | NEPC\|CRPC-Adeno DMRs |
| chr9 | 126348976 | 126353706 | 0.000176134 | −24.52059415 | Hypo-methylated | NEPC\|CRPC-Adeno DMRs |
| chr9 | 126887818 | 126888262 | 2.91E−05 | −32.2263044 | Hypo-methylated | NEPC\|CRPC-Adeno DMRs |
| chr9 | 127020722 | 127021162 | 0.000332311 | 27.10493421 | Hyper-methylated | NEPC\|CRPC-Adeno DMRs |
| chr9 | 127105180 | 127106084 | 1.98E−05 | −42.55849791 | Hypo-methylated | NEPC\|CRPC-Adeno DMRs |
| chr9 | 127265611 | 127266113 | 7.77E−09 | 25.29416063 | Hyper-methylated | NEPC\|CRPC-Adeno DMRs |
| chr9 | 127582708 | 127583244 | 0.001532655 | −21.30381335 | Hypo-methylated | NEPC\|CRPC-Adeno DMRs |
| chr9 | 127596062 | 127597353 | 0.005737741 | −34.31198769 | Hypo-methylated | NEPC\|CRPC-Adeno DMRs |
| chr9 | 128160070 | 128160127 | 0.001694183 | −44.082846 | Hypo-methylated | NEPC\|CRPC-Adeno DMRs |
| chr9 | 128819673 | 128819970 | 0.001694183 | −49.0369281 | Hypo-methylated | NEPC\|CRPC-Adeno DMRs |
| chr9 | 128984730 | 128985520 | 0.001365349 | 22.25804151 | Hyper-methylated | NEPC\|CRPC-Adeno DMRs |
| chr9 | 129276918 | 129276962 | 1.70E−05 | 30.21645898 | Hyper-methylated | NEPC\|CRPC-Adeno DMRs |
| chr9 | 129294453 | 129296986 | 0.003113626 | −26.66141498 | Hypo-methylated | NEPC\|CRPC-Adeno DMRs |
| chr9 | 129373240 | 129373646 | 0.000200845 | 21.3574269 | Hyper-methylated | NEPC\|CRPC-Adeno DMRs |
| chr9 | 129895856 | 129896382 | 5.50E−05 | −52.55407207 | Hypo-methylated | NEPC\|CRPC-Adeno DMRs |
| chr9 | 130292451 | 130292760 | 1.70E−05 | −31.25087719 | Hypo-methylated | NEPC\|CRPC-Adeno DMRs |
| chr9 | 130307535 | 130313386 | 0.000106805 | −35.34883041 | Hypo-methylated | NEPC\|CRPC-Adeno DMRs |
| chr9 | 130505114 | 130505156 | 0.001694183 | −38.98637372 | Hypo-methylated | NEPC\|CRPC-Adeno DMRs |
| chr9 | 130580413 | 130580501 | 0.001694183 | −44.4696533 | Hypo-methylated | NEPC\|CRPC-Adeno DMRs |
| chr9 | 130712087 | 130713170 | 2.14E−05 | −25.51484962 | Hypo-methylated | NEPC\|CRPC-Adeno DMRs |
| chr9 | 130737328 | 130737541 | 0.000176134 | −36.47810673 | Hypo-methylated | NEPC\|CRPC-Adeno DMRs |
| chr9 | 130829121 | 130829270 | 0.00222033 | −32.28092105 | Hypo-methylated | NEPC\|CRPC-Adeno DMRs |
| chr9 | 130891603 | 130894541 | 0.003113626 | −27.67390351 | Hypo-methylated | NEPC\|CRPC-Adeno DMRs |
| chr9 | 131127059 | 131127544 | 5.34E−09 | −29.69706266 | Hypo-methylated | NEPC\|CRPC-Adeno DMRs |
| chr9 | 131217381 | 131217558 | 0.003525992 | −38.99437135 | Hypo-methylated | NEPC\|CRPC-Adeno DMRs |
| chr9 | 131219584 | 131219746 | 0.009458442 | −30.69057018 | Hypo-methylated | NEPC\|CRPC-Adeno DMRs |
| chr9 | 132071141 | 132071528 | 0.00040075 | −26.50106326 | Hypo-methylated | NEPC\|CRPC-Adeno DMRs |
| chr9 | 132178565 | 132178692 | 1.98E−05 | −24.84628715 | Hypo-methylated | NEPC\|CRPC-Adeno DMRs |
| chr9 | 132460627 | 132472157 | 1.02E−05 | −34.81892272 | Hypo-methylated | NEPC\|CRPC-Adeno DMRs |
| chr9 | 132549343 | 132549504 | 0.000549237 | −32.08045113 | Hypo-methylated | NEPC\|CRPC-Adeno DMRs |
| chr9 | 133297469 | 133298065 | 3.19E−08 | −36.73218476 | Hypo-methylated | NEPC\|CRPC-Adeno DMRs |
| chr9 | 133411323 | 133411872 | 0.00769092 | −23.91554581 | Hypo-methylated | NEPC\|CRPC-Adeno DMRs |
| chr9 | 133770973 | 133771975 | 0.001274051 | −26.12502089 | Hypo-methylated | NEPC\|CRPC-Adeno DMRs |
| chr9 | 133776970 | 133777219 | 3.40E−05 | −25.87092471 | Hypo-methylated | NEPC\|CRPC-Adeno DMRs |
| chr9 | 133788538 | 133788804 | 5.34E−09 | −22.67605882 | Hypo-methylated | NEPC\|CRPC-Adeno DMRs |
| chr9 | 133895277 | 133895368 | 0.001694183 | −39.77105263 | Hypo-methylated | NEPC\|CRPC-Adeno DMRs |
| chr9 | 134183544 | 134183612 | 5.17E−06 | −30.82248804 | Hypo-methylated | NEPC\|CRPC-Adeno DMRs |
| chr9 | 134199569 | 134204978 | 0.005297267 | −27.40371763 | Hypo-methylated | NEPC\|CRPC-Adeno DMRs |
| chr9 | 135089560 | 135089692 | 0.001694183 | −45.04003268 | Hypo-methylated | NEPC\|CRPC-Adeno DMRs |
| chr9 | 135374946 | 135381835 | 1.09E−05 | −27.67350533 | Hypo-methylated | NEPC\|CRPC-Adeno DMRs |
| chr9 | 135419388 | 135419528 | 0.000282282 | −25.27071994 | Hypo-methylated | NEPC\|CRPC-Adeno DMRs |
| chr9 | 135608716 | 135608804 | 5.17E−06 | −38.83574087 | Hypo-methylated | NEPC\|CRPC-Adeno DMRs |
| chr9 | 135678010 | 135680846 | 0.000277187 | −25.01223893 | Hypo-methylated | NEPC\|CRPC-Adeno DMRs |
| chr9 | 136007377 | 136008637 | 5.14E−05 | −46.86939748 | Hypo-methylated | NEPC\|CRPC-Adeno DMRs |
| chr9 | 136038775 | 136038972 | 1.42E−05 | 23.5453051 | Hyper-methylated | NEPC\|CRPC-Adeno DMRs |
| chr9 | 136046683 | 136052181 | 0.000152004 | −24.81713005 | Hypo-methylated | NEPC\|CRPC-Adeno DMRs |
| chr9 | 136063379 | 136075203 | 0.000183183 | −22.49033463 | Hypo-methylated | NEPC\|CRPC-Adeno DMRs |
| chr9 | 136290366 | 136290519 | 0.001694183 | −36.98611111 | Hypo-methylated | NEPC\|CRPC-Adeno DMRs |
| chr9 | 136324006 | 136324257 | 0.000200845 | −42.48789474 | Hypo-methylated | NEPC\|CRPC-Adeno DMRs |
| chr9 | 136399391 | 136401261 | 8.50E−13 | −20.53795771 | Hypo-methylated | NEPC\|CRPC-Adeno DMRs |
| chr9 | 136529391 | 136529512 | 0.003327791 | −44.80942982 | Hypo-methylated | NEPC\|CRPC-Adeno DMRs |
| chr9 | 136740461 | 136755525 | 8.90E−06 | −21.18169757 | Hypo-methylated | NEPC\|CRPC-Adeno DMRs |
| chr9 | 136814815 | 136816138 | 8.43E−06 | −28.3933114 | Hypo-methylated | NEPC\|CRPC-Adeno DMRs |
| chr9 | 136834290 | 136838619 | 2.58E−07 | −30.66062486 | Hypo-methylated | NEPC\|CRPC-Adeno DMRs |
| chr9 | 136919324 | 136921209 | 0.003932932 | 21.68233779 | Hyper-methylated | NEPC\|CRPC-Adeno DMRs |
| chr9 | 137125231 | 137126486 | 0.00064342 | −22.05933723 | Hypo-methylated | NEPC\|CRPC-Adeno DMRs |
| chr9 | 137128024 | 137140495 | 2.19E−05 | −21.14350639 | Hypo-methylated | NEPC\|CRPC-Adeno DMRs |
| chr9 | 137174162 | 137177197 | 0.004806912 | −21.80033986 | Hypo-methylated | NEPC\|CRPC-Adeno DMRs |
| chr9 | 137186596 | 137190446 | 3.44E−07 | −29.82000646 | Hypo-methylated | NEPC\|CRPC-Adeno DMRs |
| chr9 | 137200887 | 137211391 | 2.54E−06 | −23.1531005 | Hypo-methylated | NEPC\|CRPC-Adeno DMRs |
| chr9 | 137239316 | 137240645 | 8.71E−07 | −31.32173913 | Hypo-methylated | NEPC\|CRPC-Adeno DMRs |
| chr9 | 137247145 | 137250725 | 0.000148223 | 26.32691971 | Hyper-methylated | NEPC\|CRPC-Adeno DMRs |
| chr9 | 137251149 | 137251444 | 0.000332001 | −26.0626032 | Hypo-methylated | NEPC\|CRPC-Adeno DMRs |
| chr9 | 137266220 | 137267513 | 0.000288502 | 33.11403509 | Hyper-methylated | NEPC\|CRPC-Adeno DMRs |
| chr9 | 137305867 | 137306372 | 2.91E−06 | −43.06008772 | Hypo-methylated | NEPC\|CRPC-Adeno DMRs |
| chr9 | 137354301 | 137356395 | 0.001229862 | −21.06754386 | Hypo-methylated | NEPC\|CRPC-Adeno DMRs |
| chr9 | 137366430 | 137367425 | 0.004215557 | 21.6186565 | Hyper-methylated | NEPC\|CRPC-Adeno DMRs |
| chr9 | 137529736 | 137533113 | 0.001506981 | −21.74190946 | Hypo-methylated | NEPC\|CRPC-Adeno DMRs |
| chr9 | 137756555 | 137804526 | 6.71E−14 | −27.80637636 | Hypo-methylated | NEPC\|CRPC-Adeno DMRs |
| chr9 | 137880070 | 137881685 | 0.002859601 | −27.16421053 | Hypo-methylated | NEPC\|CRPC-Adeno DMRs |
| chr9 | 137882878 | 137883201 | 8.71E−07 | −23.31157241 | Hypo-methylated | NEPC\|CRPC-Adeno DMRs |
| chr9 | 137937734 | 137962160 | 7.10E−08 | −34.27760615 | Hypo-methylated | NEPC\|CRPC-Adeno DMRs |
| chr9 | 138010698 | 138011773 | 0.00027623 | −20.57776058 | Hypo-methylated | NEPC\|CRPC-Adeno DMRs |
| chr9 | 138018421 | 138019435 | 0.000200845 | −27.26116959 | Hypo-methylated | NEPC\|CRPC-Adeno DMRs |
| chr9 | 138193278 | 138193486 | 0.001065261 | −26.79298246 | Hypo-methylated | NEPC\|CRPC-Adeno DMRs |
| chr9 | 138220976 | 138221860 | 0.001814062 | −33.75757985 | Hypo-methylated | NEPC\|CRPC-Adeno DMRs |
| chr9 | 138233063 | 138233203 | 0.000176134 | −33.29210526 | Hypo-methylated | NEPC\|CRPC-Adeno DMRs |
| chr9 | 138268102 | 138268783 | 0.000337664 | −31.44040045 | Hypo-methylated | NEPC\|CRPC-Adeno DMRs |
| chr9 | 138272060 | 138272350 | 0.000700082 | −32.93145933 | Hypo-methylated | NEPC\|CRPC-Adeno DMRs |
| chr9 | 138294954 | 138295229 | 0.004291094 | −21.78421053 | Hypo-methylated | NEPC\|CRPC-Adeno DMRs |
| chr9 | 138330554 | 138337813 | 0.000619184 | −27.43416409 | Hypo-methylated | NEPC\|CRPC-Adeno DMRs |
| chr9 | 138508627 | 138550678 | 6.09E−06 | −20.00405383 | Hypo-methylated | NEPC\|CRPC-Adeno DMRs |
| chr9 | 138597380 | 138603436 | 0.00015998 | −24.30320245 | Hypo-methylated | NEPC\|CRPC-Adeno DMRs |
| chr9 | 138609320 | 138611468 | 9.26E−06 | −25.92572255 | Hypo-methylated | NEPC\|CRPC-Adeno DMRs |
| chr9 | 138614331 | 138616090 | 0.002227988 | −20.41453982 | Hypo-methylated | NEPC\|CRPC-Adeno DMRs |
| chr9 | 138661452 | 138662609 | 1.78E−13 | −26.8234635 | Hypo-methylated | NEPC\|CRPC-Adeno DMRs |
| chr9 | 138675638 | 138678361 | 2.94E−12 | −21.09658034 | Hypo-methylated | NEPC\|CRPC-Adeno DMRs |
| chr9 | 139038780 | 139039138 | 0.00222033 | −25.42872807 | Hypo-methylated | NEPC\|CRPC-Adeno DMRs |
| chr9 | 139060477 | 139062568 | 0.008187764 | 26.53779904 | Hyper-methylated | NEPC\|CRPC-Adeno DMRs |
| chr9 | 139096816 | 139097662 | 7.90E−10 | 27.98502796 | Hyper-methylated | NEPC\|CRPC-Adeno DMRs |
| chr9 | 139141928 | 139142089 | 0.000303685 | −33.82079504 | Hypo-methylated | NEPC\|CRPC-Adeno DMRs |
| chr9 | 139155510 | 139155682 | 0.000549237 | −27.21695906 | Hypo-methylated | NEPC\|CRPC-Adeno DMRs |
| chr9 | 139396824 | 139397578 | 5.69E−07 | −22.17925804 | Hypo-methylated | NEPC\|CRPC-Adeno DMRs |
| chr9 | 139400169 | 139401496 | 4.91E−09 | −20.60446738 | Hypo-methylated | NEPC\|CRPC-Adeno DMRs |
| chr9 | 139403170 | 139403460 | 0.000439429 | −26.66684211 | Hypo-methylated | NEPC\|CRPC-Adeno DMRs |
| chr9 | 139409321 | 139411441 | 7.02E−09 | −21.25608103 | Hypo-methylated | NEPC\|CRPC-Adeno DMRs |
| chr9 | 139573150 | 139574082 | 0.000889013 | 38.90327549 | Hyper-methylated | NEPC\|CRPC-Adeno DMRs |
| chr9 | 139584634 | 139585969 | 5.47E−07 | −23.21097661 | Hypo-methylated | NEPC\|CRPC-Adeno DMRs |
| chr9 | 139635430 | 139635760 | 0.00217795 | −23.81285714 | Hypo-methylated | NEPC\|CRPC-Adeno DMRs |
| chr9 | 139648148 | 139649508 | 9.70E−06 | −22.5070255 | Hypo-methylated | NEPC\|CRPC-Adeno DMRs |
| chr9 | 139658444 | 139659183 | 0.005737741 | −43.5460213 | Hypo-methylated | NEPC\|CRPC-Adeno DMRs |
| chr9 | 139672072 | 139672185 | 5.17E−06 | 20.70924375 | Hyper-methylated | NEPC\|CRPC-Adeno DMRs |
| chr9 | 139750206 | 139751887 | 1.11E−05 | −22.62578586 | Hypo-methylated | NEPC\|CRPC-Adeno DMRs |
| chr9 | 139873366 | 139873497 | 1.27E−05 | 26.33965976 | Hyper-methylated | NEPC\|CRPC-Adeno DMRs |
| chr9 | 139902068 | 139904811 | 1.71E−19 | −31.34162092 | Hypo-methylated | NEPC\|CRPC-Adeno DMRs |
| chr9 | 139907751 | 139907914 | 9.81E−05 | 24.99418184 | Hyper-methylated | NEPC\|CRPC-Adeno DMRs |
| chr9 | 140175388 | 140175436 | 1.55E−06 | −23.27079858 | Hypo-methylated | NEPC\|CRPC-Adeno DMRs |
| chr9 | 140266895 | 140267338 | 0.000371055 | −20.58553606 | Hypo-methylated | NEPC\|CRPC-Adeno DMRs |
| chr9 | 140322962 | 140324976 | 1.63E−08 | −33.79904201 | Hypo-methylated | NEPC\|CRPC-Adeno DMRs |
| chr9 | 140373567 | 140375030 | 7.07E−07 | −37.4128193 | Hypo-methylated | NEPC\|CRPC-Adeno DMRs |
| chr10 | 527070 | 527775 | 1.43E−05 | −24.46527778 | Hypo-methylated | NEPC\|CRPC-Adeno DMRs |
| chr10 | 676740 | 676826 | 0.000549237 | −61.39158663 | Hypo-methylated | NEPC\|CRPC-Adeno DMRs |
| chr10 | 1259532 | 1260038 | 0.000489849 | 22.31520468 | Hyper-methylated | NEPC\|CRPC-Adeno DMRs |
| chr10 | 1477190 | 1477331 | 0.001694183 | −57.73962848 | Hypo-methylated | NEPC\|CRPC-Adeno DMRs |
| chr10 | 1594637 | 1595786 | 7.21E−07 | −22.14319889 | Hypo-methylated | NEPC\|CRPC-Adeno DMRs |
| chr10 | 1641115 | 1641173 | 0.000176134 | −37.97079678 | Hypo-methylated | NEPC\|CRPC-Adeno DMRs |
| chr10 | 1889183 | 1920662 | 0.004693517 | −23.84131909 | Hypo-methylated | NEPC\|CRPC-Adeno DMRs |
| chr10 | 2432059 | 2538391 | 5.79E−07 | −35.25457283 | Hypo-methylated | NEPC\|CRPC-Adeno DMRs |
| chr10 | 2932371 | 2932614 | 0.000303685 | −28.612044 | Hypo-methylated | NEPC\|CRPC-Adeno DMRs |
| chr10 | 2960341 | 2964800 | 0.001694183 | −24.39995136 | Hypo-methylated | NEPC\|CRPC-Adeno DMRs |
| chr10 | 3059801 | 3071877 | 0.001694183 | −30.66081011 | Hypo-methylated | NEPC\|CRPC-Adeno DMRs |
| chr10 | 3168192 | 3171146 | 0.000292068 | −20.18284905 | Hypo-methylated | NEPC\|CRPC-Adeno DMRs |
| chr10 | 3421354 | 3441107 | 0.001065261 | −35.79956045 | Hypo-methylated | NEPC\|CRPC-Adeno DMRs |
| chr10 | 3710872 | 3711105 | 0.001802623 | 25.86559536 | Hyper-methylated | NEPC\|CRPC-Adeno DMRs |
| chr10 | 4908800 | 4983807 | 0.001883547 | −22.70517169 | Hypo-methylated | NEPC\|CRPC-Adeno DMRs |
| chr10 | 6622144 | 6622509 | 3.16E−11 | 24.62232053 | Hyper-methylated | NEPC\|CRPC-Adeno DMRs |
| chr10 | 7253328 | 7299936 | 0.00217795 | −25.62659541 | Hypo-methylated | NEPC\|CRPC-Adeno DMRs |
| chr10 | 7317076 | 7439794 | 5.28E−11 | −23.97294806 | Hypo-methylated | NEPC\|CRPC-Adeno DMRs |
| chr10 | 7667450 | 7702969 | 1.72E−05 | −37.20499746 | Hypo-methylated | NEPC\|CRPC-Adeno DMRs |
| chr10 | 8087190 | 8087423 | 0.000200845 | −22.73597695 | Hypo-methylated | NEPC\|CRPC-Adeno DMRs |
| chr10 | 11059467 | 11060757 | 1.81E−50 | 28.59533291 | Hyper-methylated | NEPC\|CRPC-Adeno DMRs |
| chr10 | 11950223 | 11950312 | 0.001694183 | −25.97258772 | Hypo-methylated | NEPC\|CRPC-Adeno DMRs |
| chr10 | 12633277 | 12633387 | 0.001694183 | −47.75243665 | Hypo-methylated | NEPC\|CRPC-Adeno DMRs |
| chr10 | 13726467 | 13726635 | 5.17E−06 | −29.9553429 | Hypo-methylated | NEPC\|CRPC-Adeno DMRs |
| chr10 | 13862745 | 13863118 | 3.73E−07 | −33.73830931 | Hypo-methylated | NEPC\|CRPC-Adeno DMRs |
| chr10 | 16562018 | 16562311 | 5.49E−12 | 26.08136629 | Hyper-methylated | NEPC\|CRPC-Adeno DMRs |
| chr10 | 17879085 | 18385449 | 5.01E−05 | −24.95590203 | Hypo-methylated | NEPC\|CRPC-Adeno DMRs |
| chr10 | 20116635 | 20233812 | 1.70E−05 | −28.95327485 | Hypo-methylated | NEPC\|CRPC-Adeno DMRs |
| chr10 | 20251480 | 20497727 | 0.004652598 | −24.20573221 | Hypo-methylated | NEPC\|CRPC-Adeno DMRs |
| chr10 | 23460300 | 23460458 | 0.001694183 | 29.28596491 | Hyper-methylated | NEPC\|CRPC-Adeno DMRs |
| chr10 | 23462460 | 23464044 | 7.43E−12 | 24.47820632 | Hyper-methylated | NEPC\|CRPC-Adeno DMRs |
| chr10 | 23481419 | 23481657 | 3.06E−07 | 35.41544626 | Hyper-methylated | NEPC\|CRPC-Adeno DMRs |
| chr10 | 24020414 | 24497873 | 6.39E−06 | −25.30129265 | Hypo-methylated | NEPC\|CRPC-Adeno DMRs |
| chr10 | 24544079 | 24547683 | 0.003652661 | −30.04891555 | Hypo-methylated | NEPC\|CRPC-Adeno DMRs |
| chr10 | 24567210 | 24609833 | 2.74E−05 | −34.42947921 | Hypo-methylated | NEPC\|CRPC-Adeno DMRs |
| chr10 | 24635159 | 24805452 | 2.21E−10 | −26.04963223 | Hypo-methylated | NEPC\|CRPC-Adeno DMRs |
| chr10 | 25246662 | 25452380 | 3.23E−06 | −29.2184255 | Hypo-methylated | NEPC\|CRPC-Adeno DMRs |
| chr10 | 25908294 | 25935197 | 0.000337664 | −33.8094152 | Hypo-methylated | NEPC\|CRPC-Adeno DMRs |
| chr10 | 25990759 | 26032492 | 2.53E−05 | −30.43095934 | Hypo-methylated | NEPC\|CRPC-Adeno DMRs |
| chr10 | 26223207 | 26223337 | 5.71E−06 | 26.56326355 | Hyper-methylated | NEPC\|CRPC-Adeno DMRs |
| chr10 | 26506583 | 26506684 | 0.002376793 | 24.21839447 | Hyper-methylated | NEPC\|CRPC-Adeno DMRs |
| chr10 | 27181801 | 27205423 | 8.64E−05 | −25.67930627 | Hypo-methylated | NEPC\|CRPC-Adeno DMRs |
| chr10 | 28034317 | 28034444 | 0.00367696 | 26.65378788 | Hyper-methylated | NEPC\|CRPC-Adeno DMRs |
| chr10 | 28034591 | 28034646 | 0.000197871 | 20.92361111 | Hyper-methylated | NEPC\|CRPC-Adeno DMRs |
| chr10 | 28068843 | 28252767 | 1.12E−07 | −27.3188231 | Hypo-methylated | NEPC\|CRPC-Adeno DMRs |
| chr10 | 29874713 | 29874760 | 0.003113626 | −41.54166667 | Hypo-methylated | NEPC\|CRPC-Adeno DMRs |
| chr10 | 31986659 | 31986988 | 5.17E−06 | −39.97460988 | Hypo-methylated | NEPC\|CRPC-Adeno DMRs |
| chr10 | 32038939 | 32039037 | 0.004693517 | −25.10192147 | Hypo-methylated | NEPC\|CRPC-Adeno DMRs |
| chr10 | 36292146 | 36813242 | 9.76E−13 | −26.1925466 | Hypo-methylated | NEPC\|CRPC-Adeno DMRs |
| chr10 | 36909428 | 36917352 | 0.001694183 | −24.25921749 | Hypo-methylated | NEPC\|CRPC-Adeno DMRs |
| chr10 | 37100037 | 37122732 | 0.000154148 | −26.52591093 | Hypo-methylated | NEPC\|CRPC-Adeno DMRs |
| chr10 | 37611614 | 37869516 | 1.04E−06 | −22.42667054 | Hypo-methylated | NEPC\|CRPC-Adeno DMRs |
| chr10 | 42971733 | 42972953 | 0.004693517 | −21.82807837 | Hypo-methylated | NEPC\|CRPC-Adeno DMRs |
| chr10 | 43539337 | 43540899 | 4.20E−05 | −33.31139733 | Hypo-methylated | NEPC\|CRPC-Adeno DMRs |
| chr10 | 43728230 | 43732839 | 0.009458442 | −26.71296296 | Hypo-methylated | NEPC\|CRPC-Adeno DMRs |
| chr10 | 43754321 | 43761270 | 0.000397342 | −20.65451047 | Hypo-methylated | NEPC\|CRPC-Adeno DMRs |
| chr10 | 44005336 | 44039369 | 0.009285355 | −25.90823804 | Hypo-methylated | NEPC\|CRPC-Adeno DMRs |
| chr10 | 44843664 | 44877823 | 4.33E−05 | −24.04709003 | Hypo-methylated | NEPC\|CRPC-Adeno DMRs |
| chr10 | 44880996 | 44881165 | 0.000488426 | 21.15452092 | Hyper-methylated | NEPC\|CRPC-Adeno DMRs |
| chr10 | 44910283 | 44968454 | 0.003977693 | −21.30931774 | Hypo-methylated | NEPC\|CRPC-Adeno DMRs |
| chr10 | 45082817 | 45193328 | 2.01E−06 | −23.43216882 | Hypo-methylated | NEPC\|CRPC-Adeno DMRs |
| chr10 | 45471259 | 45473599 | 5.34E−05 | −33.02589286 | Hypo-methylated | NEPC\|CRPC-Adeno DMRs |
| chr10 | 45675987 | 45693533 | 4.46E−06 | −28.76102602 | Hypo-methylated | NEPC\|CRPC-Adeno DMRs |
| chr10 | 47008176 | 47008252 | 8.96E−05 | 22.40864662 | Hyper-methylated | NEPC\|CRPC-Adeno DMRs |
| chr10 | 47098668 | 47122554 | 1.70E−05 | −22.01647661 | Hypo-methylated | NEPC\|CRPC-Adeno DMRs |
| chr10 | 47555758 | 47627503 | 1.04E−06 | −28.71959518 | Hypo-methylated | NEPC\|CRPC-Adeno DMRs |
| chr10 | 47679990 | 47685634 | 1.43E−05 | −27.4970485 | Hypo-methylated | NEPC\|CRPC-Adeno DMRs |
| chr10 | 47749367 | 47757973 | 1.70E−05 | −30.66431704 | Hypo-methylated | NEPC\|CRPC-Adeno DMRs |
| chr10 | 50605557 | 50605614 | 0.000184724 | −23.02429003 | Hypo-methylated | NEPC\|CRPC-Adeno DMRs |
| chr10 | 50815083 | 50817377 | 3.72E−05 | 26.55161915 | Hyper-methylated | NEPC\|CRPC-Adeno DMRs |
| chr10 | 50819637 | 50819785 | 0.00039263 | 27.67192982 | Hyper-methylated | NEPC\|CRPC-Adeno DMRs |
| chr10 | 50976131 | 50977136 | 1.91E−15 | 28.88728862 | Hyper-methylated | NEPC\|CRPC-Adeno DMRs |
| chr10 | 54073802 | 54074386 | 9.29E−13 | 20.07016728 | Hyper-methylated | NEPC\|CRPC-Adeno DMRs |
| chr10 | 55636108 | 55651945 | 5.17E−06 | −28.01961722 | Hypo-methylated | NEPC\|CRPC-Adeno DMRs |
| chr10 | 57390896 | 57391102 | 6.32E−08 | 24.50604773 | Hyper-methylated | NEPC\|CRPC-Adeno DMRs |
| chr10 | 59601856 | 59668238 | 0.000104758 | −22.76927166 | Hypo-methylated | NEPC\|CRPC-Adeno DMRs |
| chr10 | 62760612 | 62760668 | 0.001694183 | −26.46081871 | Hypo-methylated | NEPC\|CRPC-Adeno DMRs |
| chr10 | 65801548 | 65801594 | 0.001012154 | 26.55357143 | Hyper-methylated | NEPC\|CRPC-Adeno DMRs |
| chr10 | 67815403 | 68247281 | 6.16E−06 | −23.99133131 | Hypo-methylated | NEPC\|CRPC-Adeno DMRs |
| chr10 | 68462413 | 69263006 | 2.51E−09 | −31.81456618 | Hypo-methylated | NEPC\|CRPC-Adeno DMRs |
| chr10 | 71328723 | 71329430 | 3.40E−10 | 34.55063926 | Hyper-methylated | NEPC\|CRPC-Adeno DMRs |
| chr10 | 71334685 | 71336242 | 0.000176134 | 27.14658173 | Hyper-methylated | NEPC\|CRPC-Adeno DMRs |
| chr10 | 71658478 | 71660941 | 0.009682706 | −21.23998538 | Hypo-methylated | NEPC\|CRPC-Adeno DMRs |
| chr10 | 71892103 | 71892769 | 4.50E−23 | −31.75503977 | Hypo-methylated | NEPC\|CRPC-Adeno DMRs |
| chr10 | 72141269 | 72141388 | 0.001694183 | −27.43327068 | Hypo-methylated | NEPC\|CRPC-Adeno DMRs |
| chr10 | 72695469 | 72695680 | 0.001065261 | −26.92358674 | Hypo-methylated | NEPC\|CRPC-Adeno DMRs |
| chr10 | 72764199 | 72769228 | 3.40E−10 | −32.17307853 | Hypo-methylated | NEPC\|CRPC-Adeno DMRs |
| chr10 | 72973592 | 72977379 | 1.07E−18 | −23.4529645 | Hypo-methylated | NEPC\|CRPC-Adeno DMRs |
| chr10 | 73221938 | 73221983 | 0.001694183 | −47.1376849 | Hypo-methylated | NEPC\|CRPC-Adeno DMRs |
| chr10 | 73466474 | 73466728 | 3.47E−07 | −26.95398479 | Hypo-methylated | NEPC\|CRPC-Adeno DMRs |
| chr10 | 73565665 | 73565861 | 0.000926684 | 24.39837535 | Hyper-methylated | NEPC\|CRPC-Adeno DMRs |
| chr10 | 73768415 | 73776944 | 0.000721524 | −34.45980335 | Hypo-methylated | NEPC\|CRPC-Adeno DMRs |
| chr10 | 77794566 | 77811143 | 7.47E−05 | −27.97736007 | Hypo-methylated | NEPC\|CRPC-Adeno DMRs |
| chr10 | 79276497 | 79291440 | 0.000153872 | −33.84502924 | Hypo-methylated | NEPC\|CRPC-Adeno DMRs |
| chr10 | 79702851 | 79702997 | 0.005737741 | −29.29264706 | Hypo-methylated | NEPC\|CRPC-Adeno DMRs |
| chr10 | 79978638 | 79978936 | 0.000176134 | −39.26150348 | Hypo-methylated | NEPC\|CRPC-Adeno DMRs |
| chr10 | 80673359 | 80673790 | 0.009274146 | −26.93704678 | Hypo-methylated | NEPC\|CRPC-Adeno DMRs |
| chr10 | 81266609 | 81267373 | 1.22E−05 | −47.97986037 | Hypo-methylated | NEPC\|CRPC-Adeno DMRs |
| chr10 | 82442236 | 82712731 | 2.09E−09 | −28.85927418 | Hypo-methylated | NEPC\|CRPC-Adeno DMRs |
| chr10 | 82810770 | 82959528 | 4.59E−06 | −28.00263767 | Hypo-methylated | NEPC\|CRPC-Adeno DMRs |
| chr10 | 83025089 | 83044825 | 0.005737741 | −33.24573587 | Hypo-methylated | NEPC\|CRPC-Adeno DMRs |
| chr10 | 83635030 | 83636333 | 1.79E−14 | 22.19552951 | Hyper-methylated | NEPC\|CRPC-Adeno DMRs |
| chr10 | 85321507 | 85341100 | 0.000549237 | −24.63125 | Hypo-methylated | NEPC\|CRPC-Adeno DMRs |
| chr10 | 88408252 | 88412839 | 0.003327791 | −29.78355801 | Hypo-methylated | NEPC\|CRPC-Adeno DMRs |
| chr10 | 88428117 | 88430347 | 1.03E−05 | −46.81815991 | Hypo-methylated | NEPC\|CRPC-Adeno DMRs |
| chr10 | 89167930 | 89189130 | 0.001694183 | 23.06481481 | Hyper-methylated | NEPC\|CRPC-Adeno DMRs |
| chr10 | 91295533 | 91308429 | 4.76E−13 | 24.29759861 | Hyper-methylated | NEPC\|CRPC-Adeno DMRs |
| chr10 | 97466652 | 97466951 | 0.002376793 | −34.83435907 | Hypo-methylated | NEPC\|CRPC-Adeno DMRs |
| chr10 | 98808702 | 98808813 | 0.000549237 | −56.32017544 | Hypo-methylated | NEPC\|CRPC-Adeno DMRs |
| chr10 | 98894117 | 98894245 | 0.003525992 | −37.51719577 | Hypo-methylated | NEPC\|CRPC-Adeno DMRs |
| chr10 | 99734416 | 99783986 | 2.48E−08 | 24.23893182 | Hyper-methylated | NEPC\|CRPC-Adeno DMRs |
| chr10 | 102419262 | 102419303 | 0.001694183 | 31.31370092 | Hyper-methylated | NEPC\|CRPC-Adeno DMRs |
| chr10 | 102475441 | 102475556 | 0.00041315 | 27.42522974 | Hyper-methylated | NEPC\|CRPC-Adeno DMRs |
| chr10 | 102620812 | 102626749 | 0.001549499 | −33.39010824 | Hypo-methylated | NEPC\|CRPC-Adeno DMRs |
| chr10 | 103536274 | 103536321 | 0.000542154 | 24.06356801 | Hyper-methylated | NEPC\|CRPC-Adeno DMRs |
| chr10 | 103603177 | 103603642 | 2.17E−06 | 28.88907731 | Hyper-methylated | NEPC\|CRPC-Adeno DMRs |
| chr10 | 106400259 | 106400309 | 5.50E−05 | 23.28070175 | Hyper-methylated | NEPC\|CRPC-Adeno DMRs |
| chr10 | 106400657 | 106401124 | 7.66E−06 | 23.12951945 | Hyper-methylated | NEPC\|CRPC-Adeno DMRs |
| chr10 | 106401291 | 106401323 | 5.01E−05 | 27.20317184 | Hyper-methylated | NEPC\|CRPC-Adeno DMRs |
| chr10 | 106401463 | 106401483 | 5.75E−05 | 26.79187552 | Hyper-methylated | NEPC\|CRPC-Adeno DMRs |
| chr10 | 108924195 | 108924503 | 3.69E−08 | 27.73528644 | Hyper-methylated | NEPC\|CRPC-Adeno DMRs |
| chr10 | 108924579 | 108924773 | 6.53E−07 | 30.72330634 | Hyper-methylated | NEPC\|CRPC-Adeno DMRs |
| chr10 | 110226159 | 110226465 | 0.000490451 | 20.40055137 | Hyper-methylated | NEPC\|CRPC-Adeno DMRs |
| chr10 | 111322897 | 111460079 | 8.46E−07 | −27.12751861 | Hypo-methylated | NEPC\|CRPC-Adeno DMRs |
| chr10 | 112289991 | 112290305 | 1.02E−07 | −28.45394041 | Hypo-methylated | NEPC\|CRPC-Adeno DMRs |
| chr10 | 112553722 | 112553780 | 0.001694183 | −45.92744361 | Hypo-methylated | NEPC\|CRPC-Adeno DMRs |
| chr10 | 113050404 | 113882148 | 2.08E−05 | −30.24059598 | Hypo-methylated | NEPC\|CRPC-Adeno DMRs |
| chr10 | 114051750 | 114051819 | 0.000176134 | −21.02368421 | Hypo-methylated | NEPC\|CRPC-Adeno DMRs |
| chr10 | 114123381 | 114123431 | 0.007136365 | 23.44511278 | Hyper-methylated | NEPC\|CRPC-Adeno DMRs |
| chr10 | 116061880 | 116061937 | 0.001694183 | 20.39242203 | Hyper-methylated | NEPC\|CRPC-Adeno DMRs |
| chr10 | 118387431 | 118387476 | 3.15E−05 | 23.72976608 | Hyper-methylated | NEPC\|CRPC-Adeno DMRs |
| chr10 | 118388135 | 118392715 | 0.005737741 | −21.2441242 | Hypo-methylated | NEPC\|CRPC-Adeno DMRs |
| chr10 | 118434404 | 118434551 | 7.69E−05 | −21.79605263 | Hypo-methylated | NEPC\|CRPC-Adeno DMRs |
| chr10 | 118885687 | 118885838 | 5.45E−05 | −22.91684211 | Hypo-methylated | NEPC\|CRPC-Adeno DMRs |
| chr10 | 118897464 | 118900329 | 5.31E−09 | 21.7271224 | Hyper-methylated | NEPC\|CRPC-Adeno DMRs |
| chr10 | 119297477 | 119301828 | 8.70E−09 | 21.87791904 | Hyper-methylated | NEPC\|CRPC-Adeno DMRs |
| chr10 | 119480788 | 119490967 | 0.008985252 | −20.68752437 | Hypo-methylated | NEPC\|CRPC-Adeno DMRs |
| chr10 | 119589954 | 119590054 | 0.000549237 | −46.70143742 | Hypo-methylated | NEPC\|CRPC-Adeno DMRs |
| chr10 | 121963962 | 121969475 | 0.009458442 | −21.20087719 | Hypo-methylated | NEPC\|CRPC-Adeno DMRs |
| chr10 | 123738257 | 123772457 | 3.10E−05 | −23.49736842 | Hypo-methylated | NEPC\|CRPC-Adeno DMRs |
| chr10 | 124714365 | 124724110 | 0.002976353 | 25.93878323 | Hyper-methylated | NEPC\|CRPC-Adeno DMRs |
| chr10 | 124896793 | 124897211 | 8.60E−10 | 25.1787139 | Hyper-methylated | NEPC\|CRPC-Adeno DMRs |
| chr10 | 124901913 | 124905964 | 2.28E−18 | 28.68750524 | Hyper-methylated | NEPC\|CRPC-Adeno DMRs |
| chr10 | 125650943 | 125651403 | 2.84E−14 | 25.08582325 | Hyper-methylated | NEPC\|CRPC-Adeno DMRs |
| chr10 | 125751936 | 125752100 | 0.003932932 | 26.17103543 | Hyper-methylated | NEPC\|CRPC-Adeno DMRs |
| chr10 | 125765718 | 125765795 | 0.000549237 | −25.60894954 | Hypo-methylated | NEPC\|CRPC-Adeno DMRs |
| chr10 | 126296003 | 126296287 | 0.002976353 | −26.66617586 | Hypo-methylated | NEPC\|CRPC-Adeno DMRs |
| chr10 | 126538091 | 126539042 | 0.001802623 | −21.08521303 | Hypo-methylated | NEPC\|CRPC-Adeno DMRs |
| chr10 | 126838125 | 126840800 | 0.001745755 | −22.21252724 | Hypo-methylated | NEPC\|CRPC-Adeno DMRs |
| chr10 | 127137319 | 127137384 | 0.000581135 | 29.45467836 | Hyper-methylated | NEPC\|CRPC-Adeno DMRs |
| chr10 | 127451738 | 127452093 | 1.70E−05 | −22.54380117 | Hypo-methylated | NEPC\|CRPC-Adeno DMRs |
| chr10 | 128606018 | 128607288 | 0.000286322 | −30.36311551 | Hypo-methylated | NEPC\|CRPC-Adeno DMRs |
| chr10 | 128993561 | 128994030 | 7.36E−10 | −21.13664045 | Hypo-methylated | NEPC\|CRPC-Adeno DMRs |
| chr10 | 129142456 | 129142649 | 0.000549237 | −41.67067669 | Hypo-methylated | NEPC\|CRPC-Adeno DMRs |
| chr10 | 129535708 | 129535814 | 1.87E−07 | −22.56817279 | Hypo-methylated | NEPC\|CRPC-Adeno DMRs |
| chr10 | 130185305 | 130203553 | 5.01E−05 | −33.38336118 | Hypo-methylated | NEPC\|CRPC-Adeno DMRs |
| chr10 | 130726227 | 130726448 | 0.000220189 | −31.66278564 | Hypo-methylated | NEPC\|CRPC-Adeno DMRs |
| chr10 | 130845594 | 130847047 | 0.001694183 | −24.52300195 | Hypo-methylated | NEPC\|CRPC-Adeno DMRs |
| chr10 | 130911198 | 130973417 | 0.000280041 | −31.27098555 | Hypo-methylated | NEPC\|CRPC-Adeno DMRs |
| chr10 | 131041046 | 131076334 | 6.21E−10 | −21.0904663 | Hypo-methylated | NEPC\|CRPC-Adeno DMRs |
| chr10 | 131195846 | 131213793 | 1.15E−05 | −25.51999008 | Hypo-methylated | NEPC\|CRPC-Adeno DMRs |
| chr10 | 131449283 | 131450296 | 0.000291618 | −20.53017265 | Hypo-methylated | NEPC\|CRPC-Adeno DMRs |
| chr10 | 131592555 | 131606696 | 8.77E−08 | −20.59090017 | Hypo-methylated | NEPC\|CRPC-Adeno DMRs |
| chr10 | 131609752 | 131609826 | 0.001694183 | −20.86403509 | Hypo-methylated | NEPC\|CRPC-Adeno DMRs |
| chr10 | 131667039 | 131668687 | 0.000549237 | −25.47225785 | Hypo-methylated | NEPC\|CRPC-Adeno DMRs |
| chr10 | 131735410 | 131736014 | 0.007136365 | −22.18479532 | Hypo-methylated | NEPC\|CRPC-Adeno DMRs |
| chr10 | 131771095 | 131771198 | 0.002613955 | 23.28345865 | Hyper-methylated | NEPC\|CRPC-Adeno DMRs |
| chr10 | 131771319 | 131771424 | 1.37E−07 | 22.28609023 | Hyper-methylated | NEPC\|CRPC-Adeno DMRs |
| chr10 | 132098756 | 132100151 | 6.53E−13 | 20.35492575 | Hyper-methylated | NEPC\|CRPC-Adeno DMRs |
| chr10 | 132968328 | 132968512 | 0.000101637 | −30.48451221 | Hypo-methylated | NEPC\|CRPC-Adeno DMRs |
| chr10 | 133047633 | 133047729 | 0.009274146 | −22.70305814 | Hypo-methylated | NEPC\|CRPC-Adeno DMRs |
| chr10 | 134007458 | 134007540 | 0.000291618 | 24.62911184 | Hyper-methylated | NEPC\|CRPC-Adeno DMRs |
| chr10 | 134092121 | 134095750 | 0.000155744 | −30.35127692 | Hypo-methylated | NEPC\|CRPC-Adeno DMRs |
| chr10 | 134108428 | 134108672 | 7.69E−05 | −41.02763158 | Hypo-methylated | NEPC\|CRPC-Adeno DMRs |
| chr10 | 134204570 | 134209887 | 2.90E−13 | 29.30860477 | Hyper-methylated | NEPC\|CRPC-Adeno DMRs |
| chr10 | 134251800 | 134252152 | 0.000286322 | −23.01988602 | Hypo-methylated | NEPC\|CRPC-Adeno DMRs |
| chr10 | 134280987 | 134281096 | 0.004750844 | −35.26380289 | Hypo-methylated | NEPC\|CRPC-Adeno DMRs |
| chr10 | 134377300 | 134377491 | 1.55E−06 | −28.07285575 | Hypo-methylated | NEPC\|CRPC-Adeno DMRs |
| chr10 | 134562574 | 134563201 | 2.20E−11 | −44.36843825 | Hypo-methylated | NEPC\|CRPC-Adeno DMRs |
| chr10 | 134589773 | 134590261 | 3.68E−07 | −33.7356989 | Hypo-methylated | NEPC\|CRPC-Adeno DMRs |
| chr10 | 134621538 | 134624084 | 4.23E−06 | −21.84565763 | Hypo-methylated | NEPC\|CRPC-Adeno DMRs |
| chr10 | 134756822 | 134756852 | 0.000101637 | 20.61224821 | Hyper-methylated | NEPC\|CRPC-Adeno DMRs |
| chr10 | 134943406 | 134945484 | 1.76E−05 | −30.56223501 | Hypo-methylated | NEPC\|CRPC-Adeno DMRs |
| chr10 | 134945598 | 134945659 | 0.000118201 | −21.37587719 | Hypo-methylated | NEPC\|CRPC-Adeno DMRs |
| chr10 | 134974407 | 134975410 | 5.02E−09 | −21.93487051 | Hypo-methylated | NEPC\|CRPC-Adeno DMRs |
| chr10 | 135043917 | 135043927 | 0.005737741 | 25.96037582 | Hyper-methylated | NEPC\|CRPC-Adeno DMRs |
| chr10 | 135044951 | 135045544 | 1.06E−06 | −23.55898696 | Hypo-methylated | NEPC\|CRPC-Adeno DMRs |
| chr11 | 197256 | 197598 | 1.66E−09 | −24.05792552 | Hypo-methylated | NEPC\|CRPC-Adeno DMRs |
| chr11 | 279404 | 279705 | 3.81E−11 | −20.83138652 | Hypo-methylated | NEPC\|CRPC-Adeno DMRs |
| chr11 | 528886 | 529684 | 0.000176134 | −33.35620301 | Hypo-methylated | NEPC\|CRPC-Adeno DMRs |
| chr11 | 888359 | 888730 | 0.000168572 | −24.1301208 | Hypo-methylated | NEPC\|CRPC-Adeno DMRs |
| chr11 | 1008216 | 1008447 | 1.33E−06 | −21.91329774 | Hypo-methylated | NEPC\|CRPC-Adeno DMRs |
| chr11 | 1011316 | 1011494 | 1.22E−05 | −29.04347923 | Hypo-methylated | NEPC\|CRPC-Adeno DMRs |
| chr11 | 1034806 | 1035110 | 0.000732983 | −29.5348219 | Hypo-methylated | NEPC\|CRPC-Adeno DMRs |
| chr11 | 1095846 | 1102571 | 3.41E−12 | −23.46553807 | Hypo-methylated | NEPC\|CRPC-Adeno DMRs |
| chr11 | 1124352 | 1140480 | 4.05E−08 | −25.0506208 | Hypo-methylated | NEPC\|CRPC-Adeno DMRs |
| chr11 | 1247404 | 1247533 | 0.000124569 | −23.46807992 | Hypo-methylated | NEPC\|CRPC-Adeno DMRs |
| chr11 | 1276645 | 1276744 | 0.009285355 | −24.68055556 | Hypo-methylated | NEPC\|CRPC-Adeno DMRs |
| chr11 | 1312042 | 1313320 | 8.87E−06 | 22.917154 | Hyper-methylated | NEPC\|CRPC-Adeno DMRs |
| chr11 | 1356807 | 1356930 | 0.007136365 | −20.3520259 | Hypo-methylated | NEPC\|CRPC-Adeno DMRs |
| chr11 | 1482302 | 1482428 | 5.01E−05 | 26.65070663 | Hyper-methylated | NEPC\|CRPC-Adeno DMRs |
| chr11 | 1585759 | 1585929 | 5.50E−05 | −32.64197531 | Hypo-methylated | NEPC\|CRPC-Adeno DMRs |
| chr11 | 1677611 | 1678911 | 5.50E−05 | −39.4591293 | Hypo-methylated | NEPC\|CRPC-Adeno DMRs |
| chr11 | 1769228 | 1769503 | 1.06E−05 | −29.18340259 | Hypo-methylated | NEPC\|CRPC-Adeno DMRs |
| chr11 | 1835900 | 1844729 | 1.56E−05 | −27.06006613 | Hypo-methylated | NEPC\|CRPC-Adeno DMRs |
| chr11 | 2033787 | 2035368 | 1.30E−09 | −34.91938217 | Hypo-methylated | NEPC\|CRPC-Adeno DMRs |
| chr11 | 2036865 | 2037815 | 3.36E−08 | −39.42145171 | Hypo-methylated | NEPC\|CRPC-Adeno DMRs |
| chr11 | 2115657 | 2115907 | 0.002174767 | −28.95748988 | Hypo-methylated | NEPC\|CRPC-Adeno DMRs |
| chr11 | 2180892 | 2184481 | 5.75E−05 | −21.95298591 | Hypo-methylated | NEPC\|CRPC-Adeno DMRs |
| chr11 | 2332094 | 2332569 | 0.003932932 | −31.61576743 | Hypo-methylated | NEPC\|CRPC-Adeno DMRs |
| chr11 | 2337212 | 2385429 | 3.20E−05 | −21.92916667 | Hypo-methylated | NEPC\|CRPC-Adeno DMRs |
| chr11 | 2400205 | 2411480 | 1.16E−08 | 22.97862376 | Hyper-methylated | NEPC\|CRPC-Adeno DMRs |
| chr11 | 2431518 | 2431628 | 0.000176134 | −40.48435673 | Hypo-methylated | NEPC\|CRPC-Adeno DMRs |
| chr11 | 2495135 | 2495611 | 3.15E−05 | −41.46631579 | Hypo-methylated | NEPC\|CRPC-Adeno DMRs |
| chr11 | 2580748 | 2580814 | 0.000549237 | −25.91151987 | Hypo-methylated | NEPC\|CRPC-Adeno DMRs |
| chr11 | 2595852 | 2596461 | 4.66E−05 | −31.84636223 | Hypo-methylated | NEPC\|CRPC-Adeno DMRs |
| chr11 | 2673444 | 2683858 | 1.52E−05 | −36.3245012 | Hypo-methylated | NEPC\|CRPC-Adeno DMRs |
| chr11 | 2871683 | 2871993 | 2.07E−07 | −34.89968045 | Hypo-methylated | NEPC\|CRPC-Adeno DMRs |
| chr11 | 2891062 | 2891360 | 2.14E−07 | −22.3450509 | Hypo-methylated | NEPC\|CRPC-Adeno DMRs |
| chr11 | 2954496 | 2954825 | 2.46E−05 | 22.39635628 | Hyper-methylated | NEPC\|CRPC-Adeno DMRs |
| chr11 | 3071194 | 3071338 | 0.001694183 | −28.19517544 | Hypo-methylated | NEPC\|CRPC-Adeno DMRs |
| chr11 | 3164341 | 3164534 | 0.004750844 | −30.50736216 | Hypo-methylated | NEPC\|CRPC-Adeno DMRs |
| chr11 | 4298614 | 4339096 | 0.002927544 | −24.53034534 | Hypo-methylated | NEPC\|CRPC-Adeno DMRs |
| chr11 | 4415245 | 4494345 | 0.000176134 | −24.59327216 | Hypo-methylated | NEPC\|CRPC-Adeno DMRs |
| chr11 | 8045759 | 8053872 | 0.00222033 | −21.61059942 | Hypo-methylated | NEPC\|CRPC-Adeno DMRs |
| chr11 | 8336829 | 8337252 | 0.000106852 | −23.90212097 | Hypo-methylated | NEPC\|CRPC-Adeno DMRs |
| chr11 | 10311436 | 10314121 | 0.001600753 | 23.30372807 | Hyper-methylated | NEPC\|CRPC-Adeno DMRs |
| chr11 | 11354115 | 11404893 | 5.50E−05 | −31.39355722 | Hypo-methylated | NEPC\|CRPC-Adeno DMRs |
| chr11 | 11600197 | 11600245 | 0.007271222 | −62.53050303 | Hypo-methylated | NEPC\|CRPC-Adeno DMRs |
| chr11 | 12030027 | 12030141 | 5.36E−06 | 20.2254386 | Hyper-methylated | NEPC\|CRPC-Adeno DMRs |
| chr11 | 13986005 | 13986311 | 8.65E−14 | 30.5497076 | Hyper-methylated | NEPC\|CRPC-Adeno DMRs |
| chr11 | 14277229 | 14284338 | 0.004652598 | −26.73796992 | Hypo-methylated | NEPC\|CRPC-Adeno DMRs |
| chr11 | 15638098 | 15651213 | 0.004750844 | 23.41790936 | Hyper-methylated | NEPC\|CRPC-Adeno DMRs |
| chr11 | 15962841 | 15964430 | 5.94E−08 | 24.42032668 | Hyper-methylated | NEPC\|CRPC-Adeno DMRs |
| chr11 | 16836692 | 16836916 | 0.003652661 | −20.65157895 | Hypo-methylated | NEPC\|CRPC-Adeno DMRs |
| chr11 | 17552955 | 17553237 | 0.004750844 | −34.89400585 | Hypo-methylated | NEPC\|CRPC-Adeno DMRs |
| chr11 | 17757699 | 17784810 | 1.75E−06 | 22.24869066 | Hyper-methylated | NEPC\|CRPC-Adeno DMRs |
| chr11 | 18750201 | 18765102 | 0.000687445 | −22.67991056 | Hypo-methylated | NEPC\|CRPC-Adeno DMRs |
| chr11 | 18802245 | 18812770 | 5.17E−06 | 25.84071755 | Hyper-methylated | NEPC\|CRPC-Adeno DMRs |
| chr11 | 20230414 | 20230483 | 0.000549237 | 23.49022556 | Hyper-methylated | NEPC\|CRPC-Adeno DMRs |
| chr11 | 22454301 | 22454399 | 0.002138478 | 30.68263158 | Hyper-methylated | NEPC\|CRPC-Adeno DMRs |
| chr11 | 30409847 | 30527670 | 8.05E−11 | −29.76928432 | Hypo-methylated | NEPC\|CRPC-Adeno DMRs |
| chr11 | 30680660 | 31087495 | 6.82E−07 | −21.56655405 | Hypo-methylated | NEPC\|CRPC-Adeno DMRs |
| chr11 | 32448409 | 32448774 | 4.82E−08 | 23.53344516 | Hyper-methylated | NEPC\|CRPC-Adeno DMRs |
| chr11 | 32451777 | 32451916 | 0.001802623 | −31.55545113 | Hypo-methylated | NEPC\|CRPC-Adeno DMRs |
| chr11 | 32454919 | 32458468 | 3.42E−24 | 21.33678094 | Hyper-methylated | NEPC\|CRPC-Adeno DMRs |
| chr11 | 33640110 | 33640180 | 0.000176134 | 23.67324561 | Hyper-methylated | NEPC\|CRPC-Adeno DMRs |
| chr11 | 33667283 | 33667578 | 2.27E−06 | −27.6377193 | Hypo-methylated | NEPC\|CRPC-Adeno DMRs |
| chr11 | 34852392 | 34888583 | 0.001694183 | −28.67871067 | Hypo-methylated | NEPC\|CRPC-Adeno DMRs |
| chr11 | 36476272 | 36477308 | 3.47E−07 | −22.60740741 | Hypo-methylated | NEPC\|CRPC-Adeno DMRs |
| chr11 | 37120365 | 37414157 | 0.000153872 | −22.91828453 | Hypo-methylated | NEPC\|CRPC-Adeno DMRs |
| chr11 | 41330447 | 42895559 | 5.01E−30 | −32.44839382 | Hypo-methylated | NEPC\|CRPC-Adeno DMRs |
| chr11 | 44007973 | 44013893 | 0.002336248 | −28.45933014 | Hypo-methylated | NEPC\|CRPC-Adeno DMRs |
| chr11 | 44066442 | 44072905 | 0.000160831 | −20.47175546 | Hypo-methylated | NEPC\|CRPC-Adeno DMRs |
| chr11 | 44614655 | 44614968 | 0.000549237 | −21.21823308 | Hypo-methylated | NEPC\|CRPC-Adeno DMRs |
| chr11 | 44630409 | 44630491 | 0.001694183 | −53.75263158 | Hypo-methylated | NEPC\|CRPC-Adeno DMRs |
| chr11 | 44654458 | 44661400 | 0.005737741 | 33.87792398 | Hyper-methylated | NEPC\|CRPC-Adeno DMRs |
| chr11 | 44899770 | 44916272 | 3.19E−05 | −35.34866759 | Hypo-methylated | NEPC\|CRPC-Adeno DMRs |
| chr11 | 46354292 | 46354965 | 2.53E−30 | −20.78424984 | Hypo-methylated | NEPC\|CRPC-Adeno DMRs |
| chr11 | 46384984 | 46385447 | 7.22E−05 | −21.93537275 | Hypo-methylated | NEPC\|CRPC-Adeno DMRs |
| chr11 | 47736766 | 47736972 | 2.98E−08 | −22.22385509 | Hypo-methylated | NEPC\|CRPC-Adeno DMRs |
| chr11 | 48967609 | 48990351 | 0.002859601 | −33.651368 | Hypo-methylated | NEPC\|CRPC-Adeno DMRs |
| chr11 | 57135958 | 57136124 | 0.003525992 | −39.39717349 | Hypo-methylated | NEPC\|CRPC-Adeno DMRs |
| chr11 | 57834719 | 57894793 | 7.00E−05 | −22.05773212 | Hypo-methylated | NEPC\|CRPC-Adeno DMRs |
| chr11 | 57912316 | 58297474 | 1.61E−06 | −26.91984555 | Hypo-methylated | NEPC\|CRPC-Adeno DMRs |
| chr11 | 59319776 | 59319914 | 5.50E−05 | 26.93144899 | Hyper-methylated | NEPC\|CRPC-Adeno DMRs |
| chr11 | 59787973 | 59840685 | 7.93E−06 | −23.46543862 | Hypo-methylated | NEPC\|CRPC-Adeno DMRs |
| chr11 | 59860004 | 59875844 | 0.005574264 | −21.57237981 | Hypo-methylated | NEPC\|CRPC-Adeno DMRs |
| chr11 | 60933902 | 60934092 | 0.001694183 | −37.65423977 | Hypo-methylated | NEPC\|CRPC-Adeno DMRs |
| chr11 | 61300522 | 61300774 | 1.82E−05 | −28.59911953 | Hypo-methylated | NEPC\|CRPC-Adeno DMRs |
| chr11 | 61449844 | 61449961 | 0.001694183 | −44.58991228 | Hypo-methylated | NEPC\|CRPC-Adeno DMRs |
| chr11 | 61595285 | 61596307 | 7.50E−16 | −20.84623111 | Hypo-methylated | NEPC\|CRPC-Adeno DMRs |
| chr11 | 62650164 | 62650416 | 0.003113626 | 32.88596491 | Hyper-methylated | NEPC\|CRPC-Adeno DMRs |
| chr11 | 62801810 | 63029732 | 0.006597219 | −24.49543999 | Hypo-methylated | NEPC\|CRPC-Adeno DMRs |
| chr11 | 63786577 | 63788767 | 3.60E−05 | −27.60058234 | Hypo-methylated | NEPC\|CRPC-Adeno DMRs |
| chr11 | 63812566 | 63812847 | 0.00041315 | −51.71783626 | Hypo-methylated | NEPC\|CRPC-Adeno DMRs |
| chr11 | 63851466 | 63853046 | 0.001197874 | 21.71052632 | Hyper-methylated | NEPC\|CRPC-Adeno DMRs |
| chr11 | 63900942 | 63902686 | 0.000348917 | −23.91709973 | Hypo-methylated | NEPC\|CRPC-Adeno DMRs |
| chr11 | 63987709 | 63990142 | 0.002094266 | 28.46107456 | Hyper-methylated | NEPC\|CRPC-Adeno DMRs |
| chr11 | 64003271 | 64003502 | 1.55E−06 | −36.59923246 | Hypo-methylated | NEPC\|CRPC-Adeno DMRs |
| chr11 | 64119978 | 64120743 | 1.65E−05 | −62.37964396 | Hypo-methylated | NEPC\|CRPC-Adeno DMRs |
| chr11 | 64219570 | 64313871 | 1.33E−07 | −26.04105661 | Hypo-methylated | NEPC\|CRPC-Adeno DMRs |
| chr11 | 64331702 | 64331800 | 0.000549237 | −24.72368421 | Hypo-methylated | NEPC\|CRPC-Adeno DMRs |
| chr11 | 64366483 | 64373429 | 0.001809257 | −20.29678363 | Hypo-methylated | NEPC\|CRPC-Adeno DMRs |
| chr11 | 64950007 | 64951046 | 0.003063309 | −20.8919551 | Hypo-methylated | NEPC\|CRPC-Adeno DMRs |
| chr11 | 65000766 | 65000941 | 0.007136365 | −22.99550961 | Hypo-methylated | NEPC\|CRPC-Adeno DMRs |
| chr11 | 65089430 | 65089601 | 0.00222033 | 21.36842105 | Hyper-methylated | NEPC\|CRPC-Adeno DMRs |
| chr11 | 65306024 | 65306225 | 0.001694183 | 20.13637459 | Hyper-methylated | NEPC\|CRPC-Adeno DMRs |
| chr11 | 66083698 | 66084085 | 0.00203966 | −26.63807667 | Hypo-methylated | NEPC\|CRPC-Adeno DMRs |
| chr11 | 66101563 | 66101701 | 0.000220189 | −21.19816033 | Hypo-methylated | NEPC\|CRPC-Adeno DMRs |
| chr11 | 66257043 | 66257274 | 0.000667554 | −34.50994152 | Hypo-methylated | NEPC\|CRPC-Adeno DMRs |
| chr11 | 66315693 | 66315846 | 0.001694183 | −20.84661306 | Hypo-methylated | NEPC\|CRPC-Adeno DMRs |
| chr11 | 66453951 | 66454138 | 0.001694183 | 22.96881092 | Hyper-methylated | NEPC\|CRPC-Adeno DMRs |
| chr11 | 66812205 | 66812371 | 0.000549237 | −21.18295739 | Hypo-methylated | NEPC\|CRPC-Adeno DMRs |
| chr11 | 67186073 | 67186579 | 1.33E−05 | 25.83651157 | Hyper-methylated | NEPC\|CRPC-Adeno DMRs |
| chr11 | 68084152 | 68084612 | 0.009822962 | −24.67012228 | Hypo-methylated | NEPC\|CRPC-Adeno DMRs |
| chr11 | 68200443 | 68200692 | 0.000118331 | −25.72232665 | Hypo-methylated | NEPC\|CRPC-Adeno DMRs |
| chr11 | 68417770 | 68418482 | 0.000711935 | −23.14181287 | Hypo-methylated | NEPC\|CRPC-Adeno DMRs |
| chr11 | 68431815 | 68434011 | 2.35E−05 | −24.69558897 | Hypo-methylated | NEPC\|CRPC-Adeno DMRs |
| chr11 | 68610222 | 68610744 | 0.000151874 | 22.01701223 | Hyper-methylated | NEPC\|CRPC-Adeno DMRs |
| chr11 | 68762226 | 68762360 | 0.002094266 | −24.95000334 | Hypo-methylated | NEPC\|CRPC-Adeno DMRs |
| chr11 | 68772956 | 68773145 | 1.94E−06 | −25.28082707 | Hypo-methylated | NEPC\|CRPC-Adeno DMRs |
| chr11 | 68789480 | 68790641 | 0.000667554 | −25.50926772 | Hypo-methylated | NEPC\|CRPC-Adeno DMRs |
| chr11 | 68967740 | 68968440 | 3.88E−10 | −42.31946533 | Hypo-methylated | NEPC\|CRPC-Adeno DMRs |
| chr11 | 68985397 | 69002032 | 0.003408779 | −29.13553756 | Hypo-methylated | NEPC\|CRPC-Adeno DMRs |
| chr11 | 69206477 | 69209067 | 1.13E−06 | −32.78438298 | Hypo-methylated | NEPC\|CRPC-Adeno DMRs |
| chr11 | 69259741 | 69259796 | 0.000176134 | 28.63662281 | Hyper-methylated | NEPC\|CRPC-Adeno DMRs |
| chr11 | 69512122 | 69514111 | 0.000484821 | −28.56155186 | Hypo-methylated | NEPC\|CRPC-Adeno DMRs |
| chr11 | 69551654 | 69557572 | 0.004215557 | −21.78091018 | Hypo-methylated | NEPC\|CRPC-Adeno DMRs |
| chr11 | 69630589 | 69631356 | 0.003063309 | −21.24892335 | Hypo-methylated | NEPC\|CRPC-Adeno DMRs |
| chr11 | 69678772 | 69679989 | 0.001931256 | −20.90558214 | Hypo-methylated | NEPC\|CRPC-Adeno DMRs |
| chr11 | 69718295 | 69794751 | 8.37E−09 | −23.90724236 | Hypo-methylated | NEPC\|CRPC-Adeno DMRs |
| chr11 | 69800935 | 69807960 | 4.65E−06 | −26.26264376 | Hypo-methylated | NEPC\|CRPC-Adeno DMRs |
| chr11 | 69817895 | 69818517 | 0.004750844 | −29.11052632 | Hypo-methylated | NEPC\|CRPC-Adeno DMRs |
| chr11 | 69980867 | 69981924 | 0.000340762 | −28.00964912 | Hypo-methylated | NEPC\|CRPC-Adeno DMRs |
| chr11 | 70075995 | 70090124 | 5.19E−05 | −22.04591053 | Hypo-methylated | NEPC\|CRPC-Adeno DMRs |
| chr11 | 70475562 | 70475813 | 0.001267128 | −20.77733918 | Hypo-methylated | NEPC\|CRPC-Adeno DMRs |
| chr11 | 70731820 | 70733243 | 8.61E−07 | −35.33116472 | Hypo-methylated | NEPC\|CRPC-Adeno DMRs |
| chr11 | 70795361 | 70796081 | 0.006890179 | −40.0360705 | Hypo-methylated | NEPC\|CRPC-Adeno DMRs |
| chr11 | 70862292 | 70862443 | 0.000176134 | −50.67967836 | Hypo-methylated | NEPC\|CRPC-Adeno DMRs |
| chr11 | 71022313 | 71033706 | 0.000716333 | −21.6163198 | Hypo-methylated | NEPC\|CRPC-Adeno DMRs |
| chr11 | 71080061 | 71100235 | 0.006785531 | −22.50977851 | Hypo-methylated | NEPC\|CRPC-Adeno DMRs |
| chr11 | 71937476 | 71940339 | 0.005456645 | −35.41666667 | Hypo-methylated | NEPC\|CRPC-Adeno DMRs |
| chr11 | 72301558 | 72301793 | 3.84E−07 | 25.86706419 | Hyper-methylated | NEPC\|CRPC-Adeno DMRs |
| chr11 | 72975546 | 72975797 | 3.26E−12 | −23.87120743 | Hypo-methylated | NEPC\|CRPC-Adeno DMRs |
| chr11 | 73359291 | 73359391 | 0.001694183 | −27.13596491 | Hypo-methylated | NEPC\|CRPC-Adeno DMRs |
| chr11 | 73371753 | 73372006 | 3.68E−07 | 36.36822213 | Hyper-methylated | NEPC\|CRPC-Adeno DMRs |
| chr11 | 73692155 | 73693060 | 0.000549237 | −34.40233918 | Hypo-methylated | NEPC\|CRPC-Adeno DMRs |
| chr11 | 74394246 | 74394995 | 6.36E−10 | −26.21680737 | Hypo-methylated | NEPC\|CRPC-Adeno DMRs |
| chr11 | 74408157 | 74408222 | 0.001694183 | 22.85263158 | Hyper-methylated | NEPC\|CRPC-Adeno DMRs |
| chr11 | 76463916 | 76464004 | 0.000176134 | 22.95328947 | Hyper-methylated | NEPC\|CRPC-Adeno DMRs |
| chr11 | 76885661 | 76885948 | 0.003592038 | −25.63508772 | Hypo-methylated | NEPC\|CRPC-Adeno DMRs |
| chr11 | 81455809 | 81455852 | 0.001694183 | −25.48613732 | Hypo-methylated | NEPC\|CRPC-Adeno DMRs |
| chr11 | 81662718 | 81698031 | 4.34E−06 | −27.35639901 | Hypo-methylated | NEPC\|CRPC-Adeno DMRs |
| chr11 | 82115250 | 82212627 | 7.07E−07 | −21.67440476 | Hypo-methylated | NEPC\|CRPC-Adeno DMRs |
| chr11 | 89269440 | 89300467 | 0.000549237 | −30.88646617 | Hypo-methylated | NEPC\|CRPC-Adeno DMRs |
| chr11 | 95032701 | 95032880 | 0.001694183 | −44.70138889 | Hypo-methylated | NEPC\|CRPC-Adeno DMRs |
| chr11 | 100998056 | 100998492 | 1.30E−18 | 24.34713336 | Hyper-methylated | NEPC\|CRPC-Adeno DMRs |
| chr11 | 104034648 | 104035058 | 3.71E−18 | 25.11910083 | Hyper-methylated | NEPC\|CRPC-Adeno DMRs |
| chr11 | 107529806 | 107622253 | 3.15E−05 | −35.32 | Hypo-methylated | NEPC\|CRPC-Adeno DMRs |
| chr11 | 111265963 | 111266121 | 0.000549237 | −44.52656642 | Hypo-methylated | NEPC\|CRPC-Adeno DMRs |
| chr11 | 112349185 | 112370638 | 0.000581135 | −26.21907895 | Hypo-methylated | NEPC\|CRPC-Adeno DMRs |
| chr11 | 113145782 | 113145874 | 0.001694183 | −29.30175439 | Hypo-methylated | NEPC\|CRPC-Adeno DMRs |
| chr11 | 115829505 | 115830431 | 4.46E−09 | −46.42757816 | Hypo-methylated | NEPC\|CRPC-Adeno DMRs |
| chr11 | 116033407 | 116033823 | 0.000774257 | −28.90427022 | Hypo-methylated | NEPC\|CRPC-Adeno DMRs |
| chr11 | 116069951 | 116078722 | 0.002692265 | −34.67849659 | Hypo-methylated | NEPC\|CRPC-Adeno DMRs |
| chr11 | 118662661 | 118663291 | 1.78E−10 | 22.84933266 | Hyper-methylated | NEPC\|CRPC-Adeno DMRs |
| chr11 | 119050525 | 119053027 | 5.50E−05 | −32.04736842 | Hypo-methylated | NEPC\|CRPC-Adeno DMRs |
| chr11 | 119943954 | 119944133 | 0.009458442 | −27.19473684 | Hypo-methylated | NEPC\|CRPC-Adeno DMRs |
| chr11 | 122344639 | 122427446 | 0.006890179 | −24.65322566 | Hypo-methylated | NEPC\|CRPC-Adeno DMRs |
| chr11 | 122910859 | 122910995 | 0.000832692 | −28.22858187 | Hypo-methylated | NEPC\|CRPC-Adeno DMRs |
| chr11 | 123380626 | 123380939 | 0.002094266 | −31.47205978 | Hypo-methylated | NEPC\|CRPC-Adeno DMRs |
| chr11 | 123543120 | 123543183 | 0.000488426 | −24.69267907 | Hypo-methylated | NEPC\|CRPC-Adeno DMRs |
| chr11 | 124757118 | 124765703 | 2.91E−09 | −31.44834569 | Hypo-methylated | NEPC\|CRPC-Adeno DMRs |
| chr11 | 125141267 | 125179580 | 0.005332682 | −20.66142988 | Hypo-methylated | NEPC\|CRPC-Adeno DMRs |
| chr11 | 125277805 | 125291686 | 1.09E−07 | −28.34558491 | Hypo-methylated | NEPC\|CRPC-Adeno DMRs |
| chr11 | 125299752 | 125329373 | 1.15E−05 | −28.77434211 | Hypo-methylated | NEPC\|CRPC-Adeno DMRs |
| chr11 | 126033295 | 126033544 | 0.004750844 | −28.26513158 | Hypo-methylated | NEPC\|CRPC-Adeno DMRs |
| chr11 | 126235718 | 126236163 | 0.000549237 | 35.20710109 | Hyper-methylated | NEPC\|CRPC-Adeno DMRs |
| chr11 | 126284669 | 126285823 | 0.001694183 | 20.0622807 | Hyper-methylated | NEPC\|CRPC-Adeno DMRs |
| chr11 | 126422411 | 126445080 | 0.000457314 | 24.61354288 | Hyper-methylated | NEPC\|CRPC-Adeno DMRs |
| chr11 | 128321682 | 128321859 | 1.77E−05 | −42.71965628 | Hypo-methylated | NEPC\|CRPC-Adeno DMRs |
| chr11 | 130275533 | 130287404 | 1.59E−07 | −20.76761312 | Hypo-methylated | NEPC\|CRPC-Adeno DMRs |
| chr11 | 130332450 | 130335609 | 5.84E−05 | −25.01082215 | Hypo-methylated | NEPC\|CRPC-Adeno DMRs |
| chr11 | 130357360 | 130431436 | 1.45E−06 | −20.44141589 | Hypo-methylated | NEPC\|CRPC-Adeno DMRs |
| chr11 | 130492954 | 130495819 | 0.004750844 | −24.18009815 | Hypo-methylated | NEPC\|CRPC-Adeno DMRs |
| chr11 | 130518520 | 130700487 | 3.05E−05 | −21.60884947 | Hypo-methylated | NEPC\|CRPC-Adeno DMRs |
| chr11 | 131559033 | 131561244 | 2.55E−07 | −26.61980134 | Hypo-methylated | NEPC\|CRPC-Adeno DMRs |
| chr11 | 133657404 | 133657473 | 0.007136365 | 24.72556391 | Hyper-methylated | NEPC\|CRPC-Adeno DMRs |
| chr11 | 133702710 | 133702924 | 0.000300362 | 21.28247238 | Hyper-methylated | NEPC\|CRPC-Adeno DMRs |
| chr11 | 133800810 | 133801158 | 0.001274051 | 24.43842105 | Hyper-methylated | NEPC\|CRPC-Adeno DMRs |
| chr11 | 133896652 | 133902768 | 9.80E−07 | −42.11296508 | Hypo-methylated | NEPC\|CRPC-Adeno DMRs |
| chr11 | 134237849 | 134237923 | 0.000176134 | −30.69868421 | Hypo-methylated | NEPC\|CRPC-Adeno DMRs |
| chr11 | 134408805 | 134442817 | 0.000550461 | −21.88684211 | Hypo-methylated | NEPC\|CRPC-Adeno DMRs |
| chr11 | 134454691 | 134509057 | 4.03E−10 | −29.70304349 | Hypo-methylated | NEPC\|CRPC-Adeno DMRs |
| chr11 | 134514472 | 134525696 | 0.009285355 | −20.94650037 | Hypo-methylated | NEPC\|CRPC-Adeno DMRs |
| chr11 | 134535135 | 134631475 | 5.71E−08 | −22.81926463 | Hypo-methylated | NEPC\|CRPC-Adeno DMRs |
| chr12 | 1929412 | 1930336 | 0.001802623 | −21.63242911 | Hypo-methylated | NEPC\|CRPC-Adeno DMRs |
| chr12 | 1957458 | 1957589 | 0.000549237 | −42.68766033 | Hypo-methylated | NEPC\|CRPC-Adeno DMRs |
| chr12 | 2955201 | 2955341 | 0.003113626 | −21.55847953 | Hypo-methylated | NEPC\|CRPC-Adeno DMRs |
| chr12 | 3239851 | 3240382 | 5.50E−05 | −29.58219623 | Hypo-methylated | NEPC\|CRPC-Adeno DMRs |
| chr12 | 3297463 | 3297904 | 0.002362173 | −20.44399541 | Hypo-methylated | NEPC\|CRPC-Adeno DMRs |
| chr12 | 3386251 | 3386437 | 0.001694183 | 33.99193756 | Hyper-methylated | NEPC\|CRPC-Adeno DMRs |
| chr12 | 3728462 | 3728610 | 0.000953892 | −36.09071637 | Hypo-methylated | NEPC\|CRPC-Adeno DMRs |
| chr12 | 4273900 | 4273988 | 4.74E−05 | 22.08920787 | Hyper-methylated | NEPC\|CRPC-Adeno DMRs |
| chr12 | 4378268 | 4378441 | 0.003327791 | 23.10923653 | Hyper-methylated | NEPC\|CRPC-Adeno DMRs |
| chr12 | 4480040 | 4492995 | 0.001802623 | −24.41991089 | Hypo-methylated | NEPC\|CRPC-Adeno DMRs |
| chr12 | 4918595 | 4919476 | 3.43E−16 | 25.53059903 | Hyper-methylated | NEPC\|CRPC-Adeno DMRs |
| chr12 | 5153443 | 5153914 | 1.21E−09 | 24.3617037 | Hyper-methylated | NEPC\|CRPC-Adeno DMRs |
| chr12 | 7260660 | 7261710 | 1.95E−09 | −23.85266265 | Hypo-methylated | NEPC\|CRPC-Adeno DMRs |
| chr12 | 7592815 | 7593008 | 4.45E−11 | 36.89176718 | Hyper-methylated | NEPC\|CRPC-Adeno DMRs |
| chr12 | 7910505 | 7916226 | 0.007136365 | −28.11260294 | Hypo-methylated | NEPC\|CRPC-Adeno DMRs |
| chr12 | 8033840 | 8060755 | 0.001125639 | −22.14273183 | Hypo-methylated | NEPC\|CRPC-Adeno DMRs |
| chr12 | 8125786 | 8145716 | 0.000291618 | −24.09052935 | Hypo-methylated | NEPC\|CRPC-Adeno DMRs |
| chr12 | 10564044 | 10564106 | 0.000549237 | −41.94377611 | Hypo-methylated | NEPC\|CRPC-Adeno DMRs |
| chr12 | 13045924 | 13052225 | 0.006785531 | −32.68676901 | Hypo-methylated | NEPC\|CRPC-Adeno DMRs |
| chr12 | 14134272 | 14134868 | 3.70E−06 | 24.60202777 | Hyper-methylated | NEPC\|CRPC-Adeno DMRs |
| chr12 | 22094270 | 22095220 | 1.37E−12 | 26.32084086 | Hyper-methylated | NEPC\|CRPC-Adeno DMRs |
| chr12 | 22161261 | 22189931 | 5.75E−05 | −34.20453216 | Hypo-methylated | NEPC\|CRPC-Adeno DMRs |
| chr12 | 22323985 | 22473588 | 0.000291618 | −29.4807557 | Hypo-methylated | NEPC\|CRPC-Adeno DMRs |
| chr12 | 24416016 | 24679019 | 7.89E−07 | −29.69746387 | Hypo-methylated | NEPC\|CRPC-Adeno DMRs |
| chr12 | 26379784 | 26379996 | 7.44E−08 | −32.5361403 | Hypo-methylated | NEPC\|CRPC-Adeno DMRs |
| chr12 | 28123502 | 28123939 | 2.25E−06 | 23.03826232 | Hyper-methylated | NEPC\|CRPC-Adeno DMRs |
| chr12 | 28974213 | 29291435 | 0.001644274 | −22.26318324 | Hypo-methylated | NEPC\|CRPC-Adeno DMRs |
| chr12 | 29324853 | 29426775 | 1.08E−06 | −33.5428127 | Hypo-methylated | NEPC\|CRPC-Adeno DMRs |
| chr12 | 31231771 | 31256748 | 0.008624828 | −28.22240881 | Hypo-methylated | NEPC\|CRPC-Adeno DMRs |
| chr12 | 33591887 | 33592924 | 1.41E−18 | 27.20230921 | Hyper-methylated | NEPC\|CRPC-Adeno DMRs |
| chr12 | 34488704 | 34488755 | 0.007136365 | 32.43233083 | Hyper-methylated | NEPC\|CRPC-Adeno DMRs |
| chr12 | 34495756 | 34495974 | 0.004750844 | 27.03881579 | Hyper-methylated | NEPC\|CRPC-Adeno DMRs |
| chr12 | 34503133 | 34503177 | 0.003525992 | 31.60877193 | Hyper-methylated | NEPC\|CRPC-Adeno DMRs |
| chr12 | 38546746 | 38546947 | 0.001694183 | 20.34339525 | Hyper-methylated | NEPC\|CRPC-Adeno DMRs |
| chr12 | 38555941 | 38557458 | 0.008187764 | −25.12353958 | Hypo-methylated | NEPC\|CRPC-Adeno DMRs |
| chr12 | 38557543 | 38596711 | 0.000153872 | −30.24695906 | Hypo-methylated | NEPC\|CRPC-Adeno DMRs |
| chr12 | 39879325 | 39992364 | 2.91E−06 | −35.91579521 | Hypo-methylated | NEPC\|CRPC-Adeno DMRs |
| chr12 | 40618439 | 40618848 | 1.00E−09 | 31.94746304 | Hyper-methylated | NEPC\|CRPC-Adeno DMRs |
| chr12 | 43945381 | 43945875 | 1.25E−05 | 25.86814404 | Hyper-methylated | NEPC\|CRPC-Adeno DMRs |
| chr12 | 44896639 | 45135204 | 8.86E−08 | −24.87393146 | Hypo-methylated | NEPC\|CRPC-Adeno DMRs |
| chr12 | 49659767 | 49659927 | 9.54E−05 | −23.40150929 | Hypo-methylated | NEPC\|CRPC-Adeno DMRs |
| chr12 | 52262684 | 52263396 | 2.25E−07 | −24.79140078 | Hypo-methylated | NEPC\|CRPC-Adeno DMRs |
| chr12 | 52281891 | 52283315 | 0.00040075 | −26.09894737 | Hypo-methylated | NEPC\|CRPC-Adeno DMRs |
| chr12 | 52300763 | 52301041 | 0.00223107 | 20.12497642 | Hyper-methylated | NEPC\|CRPC-Adeno DMRs |
| chr12 | 52418969 | 52419189 | 0.001065261 | 35.45707146 | Hyper-methylated | NEPC\|CRPC-Adeno DMRs |
| chr12 | 52519143 | 52519213 | 0.003113626 | −26.82004183 | Hypo-methylated | NEPC\|CRPC-Adeno DMRs |
| chr12 | 52569828 | 52569994 | 0.005737741 | −25.48732943 | Hypo-methylated | NEPC\|CRPC-Adeno DMRs |
| chr12 | 52585500 | 52585696 | 4.65E−07 | −31.23772412 | Hypo-methylated | NEPC\|CRPC-Adeno DMRs |
| chr12 | 53002960 | 53040135 | 2.07E−05 | −23.02156115 | Hypo-methylated | NEPC\|CRPC-Adeno DMRs |
| chr12 | 53454354 | 53454977 | 0.004705671 | −30.90281955 | Hypo-methylated | NEPC\|CRPC-Adeno DMRs |
| chr12 | 53590422 | 53590524 | 0.000176134 | −23.62171053 | Hypo-methylated | NEPC\|CRPC-Adeno DMRs |
| chr12 | 54343828 | 54344059 | 0.001109725 | −25.97410952 | Hypo-methylated | NEPC\|CRPC-Adeno DMRs |
| chr12 | 54396964 | 54398941 | 3.19E−06 | 20.11761266 | Hyper-methylated | NEPC\|CRPC-Adeno DMRs |
| chr12 | 54850591 | 54852450 | 0.009458442 | −20.33044591 | Hypo-methylated | NEPC\|CRPC-Adeno DMRs |
| chr12 | 63187323 | 63193732 | 0.007136365 | 22.79398496 | Hyper-methylated | NEPC\|CRPC-Adeno DMRs |
| chr12 | 66026204 | 66114697 | 0.000176134 | −45.5003655 | Hypo-methylated | NEPC\|CRPC-Adeno DMRs |
| chr12 | 66123203 | 66123293 | 0.000721524 | 24.33880572 | Hyper-methylated | NEPC\|CRPC-Adeno DMRs |
| chr12 | 66135121 | 66136135 | 8.12E−20 | 34.32846692 | Hyper-methylated | NEPC\|CRPC-Adeno DMRs |
| chr12 | 66138108 | 66213858 | 3.81E−06 | −33.47089292 | Hypo-methylated | NEPC\|CRPC-Adeno DMRs |
| chr12 | 66262214 | 66266284 | 0.000549237 | −53.15215165 | Hypo-methylated | NEPC\|CRPC-Adeno DMRs |
| chr12 | 66582856 | 66583084 | 1.11E−08 | 21.84646773 | Hyper-methylated | NEPC\|CRPC-Adeno DMRs |
| chr12 | 70970350 | 70970388 | 0.001694183 | 22.08968254 | Hyper-methylated | NEPC\|CRPC-Adeno DMRs |
| chr12 | 75089303 | 75200269 | 0.00060921 | −20.09103099 | Hypo-methylated | NEPC\|CRPC-Adeno DMRs |
| chr12 | 85306324 | 85306813 | 0.000259019 | 20.03145196 | Hyper-methylated | NEPC\|CRPC-Adeno DMRs |
| chr12 | 85473092 | 85581480 | 0.000176134 | −39.77489949 | Hypo-methylated | NEPC\|CRPC-Adeno DMRs |
| chr12 | 86237414 | 86237858 | 0.000209051 | −29.3362706 | Hypo-methylated | NEPC\|CRPC-Adeno DMRs |
| chr12 | 96602628 | 96613768 | 0.001694183 | −22.47953216 | Hypo-methylated | NEPC\|CRPC-Adeno DMRs |
| chr12 | 97441750 | 97532354 | 0.007136365 | 25.41561256 | Hyper-methylated | NEPC\|CRPC-Adeno DMRs |
| chr12 | 99139467 | 99142838 | 0.000926684 | 30.58315789 | Hyper-methylated | NEPC\|CRPC-Adeno DMRs |
| chr12 | 103358856 | 103407000 | 3.01E−06 | 22.86010284 | Hyper-methylated | NEPC\|CRPC-Adeno DMRs |
| chr12 | 105131873 | 105136501 | 0.005574264 | −34.00047847 | Hypo-methylated | NEPC\|CRPC-Adeno DMRs |
| chr12 | 106506138 | 106531772 | 0.009458442 | −27.55740086 | Hypo-methylated | NEPC\|CRPC-Adeno DMRs |
| chr12 | 108029778 | 108040091 | 1.79E−07 | −33.56366625 | Hypo-methylated | NEPC\|CRPC-Adeno DMRs |
| chr12 | 108199853 | 108230611 | 0.000100851 | −33.89356725 | Hypo-methylated | NEPC\|CRPC-Adeno DMRs |
| chr12 | 108237526 | 108237723 | 4.03E−08 | 24.2011306 | Hyper-methylated | NEPC\|CRPC-Adeno DMRs |
| chr12 | 108238515 | 108238708 | 4.45E−08 | 23.4222488 | Hyper-methylated | NEPC\|CRPC-Adeno DMRs |
| chr12 | 109948226 | 109948310 | 0.000549237 | −29.64912281 | Hypo-methylated | NEPC\|CRPC-Adeno DMRs |
| chr12 | 110115344 | 110117875 | 0.004686715 | −22.50945992 | Hypo-methylated | NEPC\|CRPC-Adeno DMRs |
| chr12 | 111394556 | 111404204 | 0.000711935 | −27.1777128 | Hypo-methylated | NEPC\|CRPC-Adeno DMRs |
| chr12 | 111506197 | 111515269 | 3.44E−08 | −20.85550682 | Hypo-methylated | NEPC\|CRPC-Adeno DMRs |
| chr12 | 111558050 | 111558231 | 0.004750844 | −33.29197995 | Hypo-methylated | NEPC\|CRPC-Adeno DMRs |
| chr12 | 111687022 | 111703556 | 0.004750844 | −31.12269576 | Hypo-methylated | NEPC\|CRPC-Adeno DMRs |
| chr12 | 111715854 | 111725628 | 7.05E−10 | −33.82908163 | Hypo-methylated | NEPC\|CRPC-Adeno DMRs |
| chr12 | 112548914 | 112550064 | 0.005737741 | 21.92966792 | Hyper-methylated | NEPC\|CRPC-Adeno DMRs |
| chr12 | 113914255 | 113914319 | 5.17E−06 | 24.71100478 | Hyper-methylated | NEPC\|CRPC-Adeno DMRs |
| chr12 | 113917518 | 113917543 | 0.001694183 | 28.05789474 | Hyper-methylated | NEPC\|CRPC-Adeno DMRs |
| chr12 | 114075873 | 114076046 | 2.52E−10 | 20.08923289 | Hyper-methylated | NEPC\|CRPC-Adeno DMRs |
| chr12 | 114116378 | 114123214 | 0.000492768 | −32.21626794 | Hypo-methylated | NEPC\|CRPC-Adeno DMRs |
| chr12 | 114282283 | 114282968 | 0.000549237 | −37.32903688 | Hypo-methylated | NEPC\|CRPC-Adeno DMRs |
| chr12 | 114487227 | 114633556 | 3.12E−05 | −24.02221341 | Hypo-methylated | NEPC\|CRPC-Adeno DMRs |
| chr12 | 114834572 | 114836047 | 0.001802623 | −20.99097744 | Hypo-methylated | NEPC\|CRPC-Adeno DMRs |
| chr12 | 114851420 | 114852038 | 1.70E−05 | −23.75159357 | Hypo-methylated | NEPC\|CRPC-Adeno DMRs |
| chr12 | 114951594 | 114970093 | 0.00357488 | −23.73801812 | Hypo-methylated | NEPC\|CRPC-Adeno DMRs |
| chr12 | 116075282 | 116075343 | 0.003327791 | −22.05160819 | Hypo-methylated | NEPC\|CRPC-Adeno DMRs |
| chr12 | 117113330 | 117113548 | 0.000549237 | −24.87894737 | Hypo-methylated | NEPC\|CRPC-Adeno DMRs |
| chr12 | 117523483 | 117532019 | 0.006785531 | −26.94665072 | Hypo-methylated | NEPC\|CRPC-Adeno DMRs |
| chr12 | 117540303 | 117549517 | 0.000151874 | −26.63814734 | Hypo-methylated | NEPC\|CRPC-Adeno DMRs |
| chr12 | 117662782 | 117722106 | 0.001594591 | −20.28931931 | Hypo-methylated | NEPC\|CRPC-Adeno DMRs |
| chr12 | 119446615 | 119447067 | 1.55E−06 | −21.14351852 | Hypo-methylated | NEPC\|CRPC-Adeno DMRs |
| chr12 | 120826946 | 120827049 | 0.001694183 | −34.66823308 | Hypo-methylated | NEPC\|CRPC-Adeno DMRs |
| chr12 | 120848612 | 120868765 | 0.004821971 | −20.51091236 | Hypo-methylated | NEPC\|CRPC-Adeno DMRs |
| chr12 | 123986856 | 123986986 | 0.001694183 | −28.88815789 | Hypo-methylated | NEPC\|CRPC-Adeno DMRs |
| chr12 | 124371625 | 124371735 | 0.000549237 | −36.78446115 | Hypo-methylated | NEPC\|CRPC-Adeno DMRs |
| chr12 | 124911808 | 124912183 | 0.000103988 | −21.54161116 | Hypo-methylated | NEPC\|CRPC-Adeno DMRs |
| chr12 | 124929567 | 124934247 | 8.95E−06 | −24.69799169 | Hypo-methylated | NEPC\|CRPC-Adeno DMRs |
| chr12 | 124947754 | 124948357 | 6.03E−06 | −51.7127743 | Hypo-methylated | NEPC\|CRPC-Adeno DMRs |
| chr12 | 125028216 | 125028330 | 0.00328044 | −23.0251462 | Hypo-methylated | NEPC\|CRPC-Adeno DMRs |
| chr12 | 125139376 | 125140409 | 0.007249297 | −22.86453388 | Hypo-methylated | NEPC\|CRPC-Adeno DMRs |
| chr12 | 125189166 | 125189284 | 0.001694183 | −23.53508772 | Hypo-methylated | NEPC\|CRPC-Adeno DMRs |
| chr12 | 125200272 | 125200381 | 0.001694183 | −36.40953947 | Hypo-methylated | NEPC\|CRPC-Adeno DMRs |
| chr12 | 125577409 | 125578404 | 8.97E−08 | −34.92924673 | Hypo-methylated | NEPC\|CRPC-Adeno DMRs |
| chr12 | 126094178 | 126158900 | 0.004583371 | −39.04993355 | Hypo-methylated | NEPC\|CRPC-Adeno DMRs |
| chr12 | 126168496 | 126168558 | 0.000118163 | 33.28259109 | Hyper-methylated | NEPC\|CRPC-Adeno DMRs |
| chr12 | 126521536 | 126628865 | 0.000496559 | −34.44690706 | Hypo-methylated | NEPC\|CRPC-Adeno DMRs |
| chr12 | 127211340 | 127211472 | 0.002026583 | 25.65476771 | Hyper-methylated | NEPC\|CRPC-Adeno DMRs |
| chr12 | 127630580 | 127630848 | 1.82E−12 | 38.30059906 | Hyper-methylated | NEPC\|CRPC-Adeno DMRs |
| chr12 | 127940182 | 127940721 | 3.12E−08 | 30.57666611 | Hyper-methylated | NEPC\|CRPC-Adeno DMRs |
| chr12 | 128751411 | 128752217 | 2.78E−09 | 22.39471323 | Hyper-methylated | NEPC\|CRPC-Adeno DMRs |
| chr12 | 129337978 | 129338632 | 2.61E−13 | 29.42718585 | Hyper-methylated | NEPC\|CRPC-Adeno DMRs |
| chr12 | 129612373 | 129612567 | 0.00060921 | −24.54083124 | Hypo-methylated | NEPC\|CRPC-Adeno DMRs |
| chr12 | 130388656 | 130388931 | 7.00E−06 | 22.9924925 | Hyper-methylated | NEPC\|CRPC-Adeno DMRs |
| chr12 | 130646462 | 130646726 | 1.14E−08 | 22.57065902 | Hyper-methylated | NEPC\|CRPC-Adeno DMRs |
| chr12 | 130646805 | 130648788 | 1.83E−19 | 24.00415024 | Hyper-methylated | NEPC\|CRPC-Adeno DMRs |
| chr12 | 130823804 | 130823918 | 5.79E−07 | 28.16022417 | Hyper-methylated | NEPC\|CRPC-Adeno DMRs |
| chr12 | 131310832 | 131311089 | 0.000549237 | 25.83118777 | Hyper-methylated | NEPC\|CRPC-Adeno DMRs |
| chr12 | 131512554 | 131513385 | 1.70E−05 | −35.57486111 | Hypo-methylated | NEPC\|CRPC-Adeno DMRs |
| chr12 | 132250701 | 132250808 | 0.001694183 | 22.53265107 | Hyper-methylated | NEPC\|CRPC-Adeno DMRs |
| chr12 | 132329721 | 132330391 | 1.70E−06 | −39.02165135 | Hypo-methylated | NEPC\|CRPC-Adeno DMRs |
| chr12 | 132348615 | 132348838 | 6.82E−05 | −23.89653621 | Hypo-methylated | NEPC\|CRPC-Adeno DMRs |
| chr12 | 132353143 | 132354330 | 0.005992671 | −21.82397661 | Hypo-methylated | NEPC\|CRPC-Adeno DMRs |
| chr12 | 132682540 | 132682679 | 0.001012154 | −20.12865497 | Hypo-methylated | NEPC\|CRPC-Adeno DMRs |
| chr12 | 132839379 | 132841040 | 3.26E−05 | −25.70280702 | Hypo-methylated | NEPC\|CRPC-Adeno DMRs |
| chr12 | 132853709 | 132854580 | 1.59E−07 | −21.02337363 | Hypo-methylated | NEPC\|CRPC-Adeno DMRs |
| chr12 | 132864172 | 132865506 | 3.32E−07 | −27.70413012 | Hypo-methylated | NEPC\|CRPC-Adeno DMRs |
| chr12 | 132924815 | 132928455 | 0.002976353 | −26.17209691 | Hypo-methylated | NEPC\|CRPC-Adeno DMRs |
| chr12 | 132984810 | 132984868 | 0.009458442 | 20.42880486 | Hyper-methylated | NEPC\|CRPC-Adeno DMRs |
| chr12 | 133019415 | 133020031 | 6.04E−07 | −22.36681025 | Hypo-methylated | NEPC\|CRPC-Adeno DMRs |
| chr12 | 133030174 | 133030467 | 0.00678631 | −21.82296651 | Hypo-methylated | NEPC\|CRPC-Adeno DMRs |
| chr12 | 133033080 | 133033695 | 2.79E−06 | −23.69813264 | Hypo-methylated | NEPC\|CRPC-Adeno DMRs |
| chr12 | 133196600 | 133197669 | 0.004222625 | −32.11548181 | Hypo-methylated | NEPC\|CRPC-Adeno DMRs |
| chr13 | 19174214 | 19174286 | 3.15E−05 | 21.85111111 | Hyper-methylated | NEPC\|CRPC-Adeno DMRs |
| chr13 | 19449235 | 19449290 | 1.55E−06 | 30.05216862 | Hyper-methylated | NEPC\|CRPC-Adeno DMRs |
| chr13 | 20138857 | 20139255 | 6.97E−06 | 24.4412594 | Hyper-methylated | NEPC\|CRPC-Adeno DMRs |
| chr13 | 21005474 | 21006389 | 5.50E−05 | −25.89185928 | Hypo-methylated | NEPC\|CRPC-Adeno DMRs |
| chr13 | 21573175 | 21576348 | 0.002094266 | −22.14859248 | Hypo-methylated | NEPC\|CRPC-Adeno DMRs |
| chr13 | 21612297 | 21623670 | 0.002976353 | −28.34768469 | Hypo-methylated | NEPC\|CRPC-Adeno DMRs |
| chr13 | 23734294 | 23734636 | 3.68E−07 | 25.30048733 | Hyper-methylated | NEPC\|CRPC-Adeno DMRs |
| chr13 | 25201116 | 25201530 | 1.41E−06 | 24.60307696 | Hyper-methylated | NEPC\|CRPC-Adeno DMRs |
| chr13 | 27060178 | 27060347 | 0.001694183 | 31.96492375 | Hyper-methylated | NEPC\|CRPC-Adeno DMRs |
| chr13 | 27317114 | 27318547 | 5.32E−07 | 24.58965186 | Hyper-methylated | NEPC\|CRPC-Adeno DMRs |
| chr13 | 27557494 | 27557543 | 0.004705671 | 20.33979532 | Hyper-methylated | NEPC\|CRPC-Adeno DMRs |
| chr13 | 27580677 | 27580740 | 0.000828392 | −25.84210526 | Hypo-methylated | NEPC\|CRPC-Adeno DMRs |
| chr13 | 28367967 | 28370672 | 1.31E−09 | 22.63522399 | Hyper-methylated | NEPC\|CRPC-Adeno DMRs |
| chr13 | 28493311 | 28493428 | 0.000184724 | 24.28538012 | Hyper-methylated | NEPC\|CRPC-Adeno DMRs |
| chr13 | 28494772 | 28495705 | 8.86E−10 | −20.67305701 | Hypo-methylated | NEPC\|CRPC-Adeno DMRs |
| chr13 | 28498276 | 28499045 | 1.04E−17 | 28.40786447 | Hyper-methylated | NEPC\|CRPC-Adeno DMRs |
| chr13 | 28501137 | 28503509 | 8.80E−11 | 23.22999641 | Hyper-methylated | NEPC\|CRPC-Adeno DMRs |
| chr13 | 28554243 | 28554680 | 3.60E−11 | 23.30557585 | Hyper-methylated | NEPC\|CRPC-Adeno DMRs |
| chr13 | 28646955 | 28656288 | 0.000950523 | −23.19245499 | Hypo-methylated | NEPC\|CRPC-Adeno DMRs |
| chr13 | 28673714 | 28674643 | 3.10E−14 | 20.72597171 | Hyper-methylated | NEPC\|CRPC-Adeno DMRs |
| chr13 | 28917208 | 28922822 | 0.003932932 | −25.79587394 | Hypo-methylated | NEPC\|CRPC-Adeno DMRs |
| chr13 | 29068202 | 29069838 | 1.48E−40 | 22.84382376 | Hyper-methylated | NEPC\|CRPC-Adeno DMRs |
| chr13 | 29106664 | 29107311 | 2.28E−19 | 43.75214868 | Hyper-methylated | NEPC\|CRPC-Adeno DMRs |
| chr13 | 30535400 | 30566977 | 5.50E−05 | −21.93820663 | Hypo-methylated | NEPC\|CRPC-Adeno DMRs |
| chr13 | 30690627 | 30699233 | 0.000832692 | −26.57361111 | Hypo-methylated | NEPC\|CRPC-Adeno DMRs |
| chr13 | 31618127 | 31618463 | 0.000601653 | 28.65332817 | Hyper-methylated | NEPC\|CRPC-Adeno DMRs |
| chr13 | 35324592 | 35324683 | 0.003525992 | −28.43684211 | Hypo-methylated | NEPC\|CRPC-Adeno DMRs |
| chr13 | 37248150 | 37248593 | 5.08E−10 | 31.76371541 | Hyper-methylated | NEPC\|CRPC-Adeno DMRs |
| chr13 | 38444116 | 38444248 | 3.29E−08 | 25.15903683 | Hyper-methylated | NEPC\|CRPC-Adeno DMRs |
| chr13 | 39046872 | 39252420 | 2.68E−06 | −31.80664 | Hypo-methylated | NEPC\|CRPC-Adeno DMRs |
| chr13 | 39261198 | 39261353 | 6.88E−08 | 22.52865079 | Hyper-methylated | NEPC\|CRPC-Adeno DMRs |
| chr13 | 39261730 | 39262166 | 7.29E−15 | 31.19543129 | Hyper-methylated | NEPC\|CRPC-Adeno DMRs |
| chr13 | 42029810 | 42029969 | 0.000101637 | −20.85125368 | Hypo-methylated | NEPC\|CRPC-Adeno DMRs |
| chr13 | 43148278 | 43148911 | 1.21E−11 | 32.67904043 | Hyper-methylated | NEPC\|CRPC-Adeno DMRs |
| chr13 | 44838189 | 44838383 | 9.60E−05 | −22.50219298 | Hypo-methylated | NEPC\|CRPC-Adeno DMRs |
| chr13 | 48459404 | 48472939 | 0.001694183 | −21.11230159 | Hypo-methylated | NEPC\|CRPC-Adeno DMRs |
| chr13 | 49792494 | 49793603 | 0.002927544 | 20.71759259 | Hyper-methylated | NEPC\|CRPC-Adeno DMRs |
| chr13 | 49879528 | 49879786 | 5.79E−07 | −47.96648148 | Hypo-methylated | NEPC\|CRPC-Adeno DMRs |
| chr13 | 58206369 | 58209192 | 2.22E−10 | 26.74165096 | Hyper-methylated | NEPC\|CRPC-Adeno DMRs |
| chr13 | 79169717 | 79177838 | 3.87E−56 | 36.82503093 | Hyper-methylated | NEPC\|CRPC-Adeno DMRs |
| chr13 | 84091037 | 84091126 | 1.60E−06 | −20.47744748 | Hypo-methylated | NEPC\|CRPC-Adeno DMRs |
| chr13 | 99111632 | 99111800 | 0.009458442 | −28.17901688 | Hypo-methylated | NEPC\|CRPC-Adeno DMRs |
| chr13 | 99189658 | 99189782 | 0.001694183 | −26.31666667 | Hypo-methylated | NEPC\|CRPC-Adeno DMRs |
| chr13 | 99218200 | 99220588 | 0.003525992 | −31.26393762 | Hypo-methylated | NEPC\|CRPC-Adeno DMRs |
| chr13 | 99306419 | 99306550 | 0.005737741 | 20.45565302 | Hyper-methylated | NEPC\|CRPC-Adeno DMRs |
| chr13 | 102068323 | 102068876 | 2.35E−18 | 23.01855024 | Hyper-methylated | NEPC\|CRPC-Adeno DMRs |
| chr13 | 102125568 | 102237941 | 0.000184724 | −31.84405458 | Hypo-methylated | NEPC\|CRPC-Adeno DMRs |
| chr13 | 103052567 | 103052726 | 8.90E−05 | 20.08855672 | Hyper-methylated | NEPC\|CRPC-Adeno DMRs |
| chr13 | 107028785 | 107028904 | 0.001694183 | −25.34522417 | Hypo-methylated | NEPC\|CRPC-Adeno DMRs |
| chrl3 | 107769800 | 107769948 | 0.005737741 | −30.93819444 | Hypo-methylated | NEPC\|CRPC-Adeno DMRs |
| chr13 | 108997521 | 109059423 | 0.001012154 | −33.07610693 | Hypo-methylated | NEPC\|CRPC-Adeno DMRs |
| chr13 | 109195809 | 109367547 | 0.000155805 | −24.94345479 | Hypo-methylated | NEPC\|CRPC-Adeno DMRs |
| chr13 | 109500957 | 109506546 | 0.001549499 | −24.42822246 | Hypo-methylated | NEPC\|CRPC-Adeno DMRs |
| chr13 | 109979889 | 109987005 | 0.002989545 | −41.29739188 | Hypo-methylated | NEPC\|CRPC-Adeno DMRs |
| chr13 | 110697879 | 110698977 | 0.001694183 | −21.65 | Hypo-methylated | NEPC\|CRPC-Adeno DMRs |
| chr13 | 110709557 | 110770642 | 0.003063309 | 38.63523231 | Hyper-methylated | NEPC\|CRPC-Adeno DMRs |
| chr13 | 111138535 | 111145024 | 0.00357488 | −25.90923765 | Hypo-methylated | NEPC\|CRPC-Adeno DMRs |
| chr13 | 111210039 | 111210491 | 4.65E−07 | −38.71063037 | Hypo-methylated | NEPC\|CRPC-Adeno DMRs |
| chr13 | 111249550 | 111249675 | 0.001694183 | 25.98040936 | Hyper-methylated | NEPC\|CRPC-Adeno DMRs |
| chr13 | 111774082 | 111774714 | 0.003327791 | −31.41907895 | Hypo-methylated | NEPC\|CRPC-Adeno DMRs |
| chr13 | 112610659 | 112610674 | 0.000549237 | 20.12440476 | Hyper-methylated | NEPC\|CRPC-Adeno DMRs |
| chr13 | 112706777 | 112710294 | 6.69E−14 | 31.61632392 | Hyper-methylated | NEPC\|CRPC-Adeno DMRs |
| chr13 | 112756649 | 112761807 | 1.11E−26 | 35.92489084 | Hyper-methylated | NEPC\|CRPC-Adeno DMRs |
| chr13 | 112985166 | 112985628 | 1.06E−14 | 20.46701092 | Hyper-methylated | NEPC\|CRPC-Adeno DMRs |
| chr13 | 113105783 | 113105963 | 0.000164694 | −30.03282651 | Hypo-methylated | NEPC\|CRPC-Adeno DMRs |
| chr13 | 113111797 | 113112291 | 7.40E−07 | −26.55601869 | Hypo-methylated | NEPC\|CRPC-Adeno DMRs |
| chr13 | 113545793 | 113545935 | 1.70E−05 | −41.28986928 | Hypo-methylated | NEPC\|CRPC-Adeno DMRs |
| chr13 | 113637735 | 113640302 | 3.82E−08 | −35.70398122 | Hypo-methylated | NEPC\|CRPC-Adeno DMRs |
| chr13 | 113661942 | 113662108 | 0.000549237 | −30.17202381 | Hypo-methylated | NEPC\|CRPC-Adeno DMRs |
| chr13 | 113784451 | 113784533 | 5.17E−06 | 25.71339713 | Hyper-methylated | NEPC\|CRPC-Adeno DMRs |
| chr13 | 113793462 | 113793627 | 0.007136365 | −30.42857143 | Hypo-methylated | NEPC\|CRPC-Adeno DMRs |
| chr13 | 114283062 | 114285966 | 0.002239175 | −26.43924709 | Hypo-methylated | NEPC\|CRPC-Adeno DMRs |
| chr13 | 114533721 | 114533836 | 0.005737741 | −25.71208577 | Hypo-methylated | NEPC\|CRPC-Adeno DMRs |
| chr13 | 114558328 | 114558496 | 0.000549237 | 29.26369495 | Hyper-methylated | NEPC\|CRPC-Adeno DMRs |
| chr13 | 114638218 | 114638757 | 0.00060921 | −33.65776 | Hypo-methylated | NEPC\|CRPC-Adeno DMRs |
| chr13 | 114778607 | 114780382 | 5.88E−08 | −20.71372814 | Hypo-methylated | NEPC\|CRPC-Adeno DMRs |
| chr13 | 114799776 | 114800335 | 0.000291618 | 20.71814442 | Hyper-methylated | NEPC\|CRPC-Adeno DMRs |
| chr13 | 114801102 | 114805226 | 0.007958919 | −21.35909912 | Hypo-methylated | NEPC\|CRPC-Adeno DMRs |
| chr13 | 114873407 | 114875406 | 9.90E−08 | −30.16654483 | Hypo-methylated | NEPC\|CRPC-Adeno DMRs |
| chr13 | 114977017 | 114977291 | 1.70E−05 | −23.27244898 | Hypo-methylated | NEPC\|CRPC-Adeno DMRs |
| chr14 | 19422748 | 19625821 | 7.81E−09 | −33.963918 | Hypo-methylated | NEPC\|CRPC-Adeno DMRs |
| chr14 | 19890076 | 20670542 | 3.98E−21 | −31.47684869 | Hypo-methylated | NEPC\|CRPC-Adeno DMRs |
| chr14 | 20684520 | 20686496 | 2.91E−05 | −42.38163876 | Hypo-methylated | NEPC\|CRPC-Adeno DMRs |
| chr14 | 21193959 | 21304447 | 5.45E−06 | −31.36505053 | Hypo-methylated | NEPC\|CRPC-Adeno DMRs |
| chr14 | 21486315 | 21486540 | 5.50E−05 | −23.50798292 | Hypo-methylated | NEPC\|CRPC-Adeno DMRs |
| chr14 | 23189101 | 23214654 | 6.82E−05 | −20.18680837 | Hypo-methylated | NEPC\|CRPC-Adeno DMRs |
| chr14 | 23449995 | 23450432 | 1.22E−05 | −21.63040936 | Hypo-methylated | NEPC\|CRPC-Adeno DMRs |
| chr14 | 23702016 | 23730289 | 0.001134201 | −22.05509093 | Hypo-methylated | NEPC\|CRPC-Adeno DMRs |
| chr14 | 23828108 | 23834024 | 8.78E−05 | −23.56539627 | Hypo-methylated | NEPC\|CRPC-Adeno DMRs |
| chr14 | 24046272 | 24046600 | 1.09E−12 | −23.19279778 | Hypo-methylated | NEPC\|CRPC-Adeno DMRs |
| chr14 | 24459410 | 24459549 | 0.000168572 | −28.65345666 | Hypo-methylated | NEPC\|CRPC-Adeno DMRs |
| chr14 | 24506965 | 24507014 | 0.001802623 | −32.00205415 | Hypo-methylated | NEPC\|CRPC-Adeno DMRs |
| chr14 | 24601571 | 24601796 | 5.01E−05 | −21.0961501 | Hypo-methylated | NEPC\|CRPC-Adeno DMRs |
| chr14 | 25573237 | 25605661 | 0.004750844 | −20.61105705 | Hypo-methylated | NEPC\|CRPC-Adeno DMRs |
| chr14 | 36973223 | 36978196 | 1.08E−16 | 27.22566718 | Hyper-methylated | NEPC\|CRPC-Adeno DMRs |
| chr14 | 42151114 | 42583081 | 5.41E−11 | 20.86843318 | Hyper-methylated | NEPC\|CRPC-Adeno DMRs |
| chr14 | 45804587 | 45830153 | 0.000953892 | −32.95357327 | Hypo-methylated | NEPC\|CRPC-Adeno DMRs |
| chr14 | 51326366 | 51326421 | 0.001694183 | −40.1462406 | Hypo-methylated | NEPC\|CRPC-Adeno DMRs |
| chr14 | 52535699 | 52536482 | 1.34E−13 | 23.25635154 | Hyper-methylated | NEPC\|CRPC-Adeno DMRs |
| chr14 | 54417051 | 54417301 | 0.001249757 | −23.98531514 | Hypo-methylated | NEPC\|CRPC-Adeno DMRs |
| chr14 | 58061599 | 58323760 | 8.77E−08 | −37.57646973 | Hypo-methylated | NEPC\|CRPC-Adeno DMRs |
| chr14 | 58332141 | 58333120 | 6.91E−22 | 23.67245656 | Hyper-methylated | NEPC\|CRPC-Adeno DMRs |
| chr14 | 60386788 | 60387033 | 0.000336992 | −21.07293233 | Hypo-methylated | NEPC\|CRPC-Adeno DMRs |
| chr14 | 61746805 | 61747606 | 8.10E−31 | −20.41793687 | Hypo-methylated | NEPC\|CRPC-Adeno DMRs |
| chr14 | 63203242 | 63248299 | 0.001012154 | 23.64328679 | Hyper-methylated | NEPC\|CRPC-Adeno DMRs |
| chr14 | 65068491 | 65068920 | 0.000340762 | 23.12473684 | Hyper-methylated | NEPC\|CRPC-Adeno DMRs |
| chr14 | 65409311 | 65409366 | 0.001694183 | −40.64366472 | Hypo-methylated | NEPC\|CRPC-Adeno DMRs |
| chr14 | 73706304 | 73706454 | 3.57E−08 | 25.72165992 | Hyper-methylated | NEPC\|CRPC-Adeno DMRs |
| chr14 | 73717025 | 73718749 | 0.006031403 | −21.80247871 | Hypo-methylated | NEPC\|CRPC-Adeno DMRs |
| chr14 | 73736869 | 73736912 | 5.01E−05 | −41.30811404 | Hypo-methylated | NEPC\|CRPC-Adeno DMRs |
| chr14 | 74103313 | 74110894 | 0.009458442 | −28.49255246 | Hypo-methylated | NEPC\|CRPC-Adeno DMRs |
| chr14 | 74218324 | 74222044 | 0.005737741 | 35.12399749 | Hyper-methylated | NEPC\|CRPC-Adeno DMRs |
| chr14 | 74706759 | 74707668 | 9.72E−19 | 24.02879328 | Hyper-methylated | NEPC\|CRPC-Adeno DMRs |
| chr14 | 74824286 | 74832564 | 1.98E−09 | −31.18950231 | Hypo-methylated | NEPC\|CRPC-Adeno DMRs |
| chr14 | 75018880 | 75019063 | 0.000488426 | 23.82943688 | Hyper-methylated | NEPC\|CRPC-Adeno DMRs |
| chr14 | 75389480 | 75389686 | 0.000101637 | −23.53976608 | Hypo-methylated | NEPC\|CRPC-Adeno DMRs |
| chr14 | 75445246 | 75447984 | 6.82E−05 | −27.10748544 | Hypo-methylated | NEPC\|CRPC-Adeno DMRs |
| chr14 | 75919734 | 75920280 | 0.000832692 | 23.29276316 | Hyper-methylated | NEPC\|CRPC-Adeno DMRs |
| chr14 | 76597532 | 76597987 | 0.000209051 | 21.01052632 | Hyper-methylated | NEPC\|CRPC-Adeno DMRs |
| chr14 | 76957095 | 76957201 | 0.000176134 | −51.01171418 | Hypo-methylated | NEPC\|CRPC-Adeno DMRs |
| chr14 | 77386556 | 77391489 | 0.002094266 | −31.31745152 | Hypo-methylated | NEPC\|CRPC-Adeno DMRs |
| chr14 | 80931119 | 80931196 | 0.001694183 | −38.0962963 | Hypo-methylated | NEPC\|CRPC-Adeno DMRs |
| chr14 | 82020400 | 82213368 | 9.99E−08 | −27.40325879 | Hypo-methylated | NEPC\|CRPC-Adeno DMRs |
| chr14 | 85996157 | 85999360 | 3.84E−14 | 28.49799338 | Hyper-methylated | NEPC\|CRPC-Adeno DMRs |
| chr14 | 90091472 | 90091976 | 0.000832692 | −55.34983215 | Hypo-methylated | NEPC\|CRPC-Adeno DMRs |
| chr14 | 90988577 | 90989764 | 0.007136365 | −25.68016917 | Hypo-methylated | NEPC\|CRPC-Adeno DMRs |
| chr14 | 91270253 | 91270445 | 0.000303685 | 27.05003249 | Hyper-methylated | NEPC\|CRPC-Adeno DMRs |
| chr14 | 93158631 | 93160889 | 0.009285355 | −25.14350329 | Hypo-methylated | NEPC\|CRPC-Adeno DMRs |
| chr14 | 93363673 | 93376490 | 0.004750844 | 21.97654922 | Hyper-methylated | NEPC\|CRPC-Adeno DMRs |
| chr14 | 93383345 | 93383521 | 0.001694183 | −30.40153867 | Hypo-methylated | NEPC\|CRPC-Adeno DMRs |
| chr14 | 93389472 | 93389558 | 0.001694183 | −22.28855472 | Hypo-methylated | NEPC\|CRPC-Adeno DMRs |
| chr14 | 93423463 | 93427368 | 1.84E−13 | −50.5182402 | Hypo-methylated | NEPC\|CRPC-Adeno DMRs |
| chr14 | 93483952 | 93484010 | 5.17E−06 | −30.01866029 | Hypo-methylated | NEPC\|CRPC-Adeno DMRs |
| chr14 | 93797948 | 93797997 | 0.001802623 | 20.02354108 | Hyper-methylated | NEPC\|CRPC-Adeno DMRs |
| chr14 | 94291582 | 94330545 | 0.000637678 | −22.94855521 | Hypo-methylated | NEPC\|CRPC-Adeno DMRs |
| chr14 | 94365235 | 94369524 | 0.000488426 | −21.00266407 | Hypo-methylated | NEPC\|CRPC-Adeno DMRs |
| chr14 | 94393497 | 94394899 | 0.000185897 | −26.94037803 | Hypo-methylated | NEPC\|CRPC-Adeno DMRs |
| chr14 | 94612526 | 94619722 | 0.000549237 | −23.58270677 | Hypo-methylated | NEPC\|CRPC-Adeno DMRs |
| chr14 | 94735621 | 94849240 | 2.61E−07 | −24.04845969 | Hypo-methylated | NEPC\|CRPC-Adeno DMRs |
| chr14 | 94953877 | 95010686 | 8.02E−06 | −26.86749761 | Hypo-methylated | NEPC\|CRPC-Adeno DMRs |
| chr14 | 95011608 | 95035504 | 9.05E−06 | −36.07893942 | Hypo-methylated | NEPC\|CRPC-Adeno DMRs |
| chr14 | 95087354 | 95150519 | 3.61E−05 | −24.01120605 | Hypo-methylated | NEPC\|CRPC-Adeno DMRs |
| chr14 | 95251457 | 95319202 | 4.74E−05 | −23.0485513 | Hypo-methylated | NEPC\|CRPC-Adeno DMRs |
| chr14 | 95331232 | 95354277 | 0.000721524 | −23.2340081 | Hypo-methylated | NEPC\|CRPC-Adeno DMRs |
| chr14 | 96128330 | 96128404 | 0.000549237 | −24.24185464 | Hypo-methylated | NEPC\|CRPC-Adeno DMRs |
| chr14 | 96557302 | 96558620 | 0.000291618 | −27.14759436 | Hypo-methylated | NEPC\|CRPC-Adeno DMRs |
| chr14 | 98651725 | 98811064 | 1.33E−05 | −25.14776836 | Hypo-methylated | NEPC\|CRPC-Adeno DMRs |
| chr14 | 99642636 | 99655632 | 0.000949885 | −20.91748119 | Hypo-methylated | NEPC\|CRPC-Adeno DMRs |
| chr14 | 99740586 | 99740613 | 0.001694183 | 21.11754386 | Hyper-methylated | NEPC\|CRPC-Adeno DMRs |
| chr14 | 99834140 | 99848696 | 0.00198814 | −24.71086079 | Hypo-methylated | NEPC\|CRPC-Adeno DMRs |
| chr14 | 100938379 | 100989443 | 0.009458442 | −50.45715678 | Hypo-methylated | NEPC\|CRPC-Adeno DMRs |
| chr14 | 101058846 | 101058987 | 0.001012154 | 27.91649958 | Hyper-methylated | NEPC\|CRPC-Adeno DMRs |
| chr14 | 101158251 | 101159582 | 2.04E−06 | −42.48606811 | Hypo-methylated | NEPC\|CRPC-Adeno DMRs |
| chr14 | 101363192 | 101365077 | 0.008985252 | −26.07134503 | Hypo-methylated | NEPC\|CRPC-Adeno DMRs |
| chr14 | 101986125 | 102012010 | 3.02E−14 | −31.22814575 | Hypo-methylated | NEPC\|CRPC-Adeno DMRs |
| chr14 | 102027154 | 102027192 | 1.70E−05 | 21.48571429 | Hyper-methylated | NEPC\|CRPC-Adeno DMRs |
| chr14 | 102047819 | 102050641 | 1.60E−05 | −21.86576859 | Hypo-methylated | NEPC\|CRPC-Adeno DMRs |
| chr14 | 102197703 | 102226402 | 0.000549237 | −31.16620964 | Hypo-methylated | NEPC\|CRPC-Adeno DMRs |
| chr14 | 102689127 | 102689423 | 3.22E−14 | −34.19484127 | Hypo-methylated | NEPC\|CRPC-Adeno DMRs |
| chr14 | 103010694 | 103011383 | 5.25E−18 | −20.61947808 | Hypo-methylated | NEPC\|CRPC-Adeno DMRs |
| chr14 | 103011438 | 103011716 | 8.77E−08 | −29.76934343 | Hypo-methylated | NEPC\|CRPC-Adeno DMRs |
| chr14 | 103286673 | 103289065 | 0.001600753 | 21.00131579 | Hyper-methylated | NEPC\|CRPC-Adeno DMRs |
| chr14 | 103550414 | 103553881 | 7.31E−13 | −30.56743224 | Hypo-methylated | NEPC\|CRPC-Adeno DMRs |
| chr14 | 103630505 | 103645543 | 0.006675101 | −22.12105263 | Hypo-methylated | NEPC\|CRPC-Adeno DMRs |
| chr14 | 103655750 | 103656106 | 0.000154411 | 22.65448952 | Hyper-methylated | NEPC\|CRPC-Adeno DMRs |
| chr14 | 103687146 | 103687320 | 1.37E−07 | 27.00731793 | Hyper-methylated | NEPC\|CRPC-Adeno DMRs |
| chr14 | 103698297 | 103699223 | 0.002094266 | −35.21299342 | Hypo-methylated | NEPC\|CRPC-Adeno DMRs |
| chr14 | 104394681 | 104394954 | 3.99E−10 | 20.82502957 | Hyper-methylated | NEPC\|CRPC-Adeno DMRs |
| chr14 | 104457910 | 104499616 | 0.000176134 | −27.06447368 | Hypo-methylated | NEPC\|CRPC-Adeno DMRs |
| chr14 | 104618099 | 104621193 | 1.70E−05 | −30.41296053 | Hypo-methylated | NEPC\|CRPC-Adeno DMRs |
| chr14 | 104624762 | 104627064 | 3.37E−09 | −26.0267441 | Hypo-methylated | NEPC\|CRPC-Adeno DMRs |
| chr14 | 104630051 | 104636744 | 5.19E−11 | −22.77658337 | Hypo-methylated | NEPC\|CRPC-Adeno DMRs |
| chr14 | 104640074 | 104643210 | 2.52E−22 | −22.61808763 | Hypo-methylated | NEPC\|CRPC-Adeno DMRs |
| chr14 | 104648223 | 104649089 | 6.16E−05 | −24.67058897 | Hypo-methylated | NEPC\|CRPC-Adeno DMRs |
| chr14 | 104772686 | 104783311 | 9.40E−08 | −22.71488319 | Hypo-methylated | NEPC\|CRPC-Adeno DMRs |
| chr14 | 104825984 | 104827169 | 3.46E−09 | −31.19064327 | Hypo-methylated | NEPC\|CRPC-Adeno DMRs |
| chr14 | 104857303 | 104859403 | 0.000149914 | −23.33632055 | Hypo-methylated | NEPC\|CRPC-Adeno DMRs |
| chr14 | 104899135 | 104901328 | 3.07E−14 | −24.2237406 | Hypo-methylated | NEPC\|CRPC-Adeno DMRs |
| chr14 | 105044399 | 105046248 | 0.000490451 | −20.84824561 | Hypo-methylated | NEPC\|CRPC-Adeno DMRs |
| chr14 | 105064538 | 105065085 | 0.007136365 | −27.51353383 | Hypo-methylated | NEPC\|CRPC-Adeno DMRs |
| chr14 | 105156942 | 105158328 | 9.54E−05 | 28.6494883 | Hyper-methylated | NEPC\|CRPC-Adeno DMRs |
| chr14 | 105181963 | 105182533 | 2.91E−05 | 31.55674034 | Hyper-methylated | NEPC\|CRPC-Adeno DMRs |
| chr14 | 105242373 | 105242617 | 0.001065261 | 29.99115524 | Hyper-methylated | NEPC\|CRPC-Adeno DMRs |
| chr14 | 105251468 | 105251769 | 0.000667554 | −26.99923977 | Hypo-methylated | NEPC\|CRPC-Adeno DMRs |
| chr14 | 105344651 | 105345191 | 4.29E−06 | −27.78643845 | Hypo-methylated | NEPC\|CRPC-Adeno DMRs |
| chr14 | 105347562 | 105347964 | 3.81E−05 | −54.36160594 | Hypo-methylated | NEPC\|CRPC-Adeno DMRs |
| chr14 | 105362126 | 105362184 | 0.000176134 | −42.43289474 | Hypo-methylated | NEPC\|CRPC-Adeno DMRs |
| chr14 | 105500756 | 105509377 | 2.10E−10 | −30.16119302 | Hypo-methylated | NEPC\|CRPC-Adeno DMRs |
| chr14 | 105554158 | 105554991 | 1.61E−10 | −25.11032442 | Hypo-methylated | NEPC\|CRPC-Adeno DMRs |
| chr14 | 105830612 | 105830650 | 2.46E−05 | 37.64473982 | Hyper-methylated | NEPC\|CRPC-Adeno DMRs |
| chr14 | 105961903 | 105962858 | 0.001012154 | 23.21804511 | Hyper-methylated | NEPC\|CRPC-Adeno DMRs |
| chr14 | 106038977 | 106040449 | 0.000439429 | −26.10527692 | Hypo-methylated | NEPC\|CRPC-Adeno DMRs |
| chr14 | 106235583 | 106241590 | 3.84E−06 | −20.31707182 | Hypo-methylated | NEPC\|CRPC-Adeno DMRs |
| chr14 | 106285172 | 106304973 | 1.70E−09 | −27.52011163 | Hypo-methylated | NEPC\|CRPC-Adeno DMRs |
| chr14 | 106319346 | 106319731 | 0.002976353 | −31.6772437 | Hypo-methylated | NEPC\|CRPC-Adeno DMRs |
| chr14 | 106367919 | 106368202 | 0.00217795 | −29.55339783 | Hypo-methylated | NEPC\|CRPC-Adeno DMRs |
| chr14 | 106654887 | 106660659 | 3.74E−05 | −29.76842105 | Hypo-methylated | NEPC\|CRPC-Adeno DMRs |
| chr14 | 106743690 | 106854924 | 2.68E−07 | −26.35797017 | Hypo-methylated | NEPC\|CRPC-Adeno DMRs |
| chr15 | 20054177 | 20142037 | 0.000716662 | −29.29495229 | Hypo-methylated | NEPC\|CRPC-Adeno DMRs |
| chr15 | 20153587 | 20488015 | 3.00E−13 | −33.13069745 | Hypo-methylated | NEPC\|CRPC-Adeno DMRs |
| chr15 | 22545642 | 22545700 | 4.65E−07 | 20.45515963 | Hyper-methylated | NEPC\|CRPC-Adeno DMRs |
| chr15 | 22547332 | 22547924 | 9.54E−05 | −20.6419711 | Hypo-methylated | NEPC\|CRPC-Adeno DMRs |
| chr15 | 22559825 | 22581983 | 4.65E−07 | −40.35538847 | Hypo-methylated | NEPC\|CRPC-Adeno DMRs |
| chr15 | 22803310 | 22803379 | 0.001694183 | −36.8754386 | Hypo-methylated | NEPC\|CRPC-Adeno DMRs |
| chr15 | 22923774 | 22927776 | 0.003327791 | −27.63540448 | Hypo-methylated | NEPC\|CRPC-Adeno DMRs |
| chr15 | 23619053 | 23657320 | 0.005557404 | −20.41249606 | Hypo-methylated | NEPC\|CRPC-Adeno DMRs |
| chr15 | 23810598 | 23811532 | 0.000153872 | −31.20089708 | Hypo-methylated | NEPC\|CRPC-Adeno DMRs |
| chr15 | 24048159 | 24105920 | 7.21E−06 | −28.59838659 | Hypo-methylated | NEPC\|CRPC-Adeno DMRs |
| chr15 | 25450823 | 25463427 | 0.000280098 | −20.61881073 | Hypo-methylated | NEPC\|CRPC-Adeno DMRs |
| chr15 | 25857969 | 25905019 | 0.000414691 | −24.02490839 | Hypo-methylated | NEPC\|CRPC-Adeno DMRs |
| chr15 | 25921423 | 25926215 | 1.77E−05 | −28.13142028 | Hypo-methylated | NEPC\|CRPC-Adeno DMRs |
| chr15 | 25940387 | 26036070 | 8.96E−16 | −23.71918589 | Hypo-methylated | NEPC\|CRPC-Adeno DMRs |
| chr15 | 26050274 | 26081122 | 0.001545937 | −26.97347017 | Hypo-methylated | NEPC\|CRPC-Adeno DMRs |
| chr15 | 26111319 | 26179895 | 1.05E−05 | −28.90601713 | Hypo-methylated | NEPC\|CRPC-Adeno DMRs |
| chr15 | 26234883 | 26251784 | 0.00020815 | −22.43097118 | Hypo-methylated | NEPC\|CRPC-Adeno DMRs |
| chr15 | 27027045 | 27027136 | 0.000549237 | −25.30886039 | Hypo-methylated | NEPC\|CRPC-Adeno DMRs |
| chr15 | 27112880 | 27113282 | 2.48E−10 | 24.13362298 | Hyper-methylated | NEPC\|CRPC-Adeno DMRs |
| chr15 | 27113411 | 27113512 | 0.004750844 | 23.22631579 | Hyper-methylated | NEPC\|CRPC-Adeno DMRs |
| chr15 | 27561338 | 27574056 | 9.13E−07 | −26.70450292 | Hypo-methylated | NEPC\|CRPC-Adeno DMRs |
| chr15 | 28051002 | 28051102 | 0.001694183 | 46.99587162 | Hyper-methylated | NEPC\|CRPC-Adeno DMRs |
| chr15 | 29211632 | 29211729 | 1.70E−05 | −45.21989471 | Hypo-methylated | NEPC\|CRPC-Adeno DMRs |
| chr15 | 29310304 | 29310691 | 0.000100851 | −33.66814195 | Hypo-methylated | NEPC\|CRPC-Adeno DMRs |
| chr15 | 29675754 | 29684327 | 0.000581135 | −23.40071011 | Hypo-methylated | NEPC\|CRPC-Adeno DMRs |
| chr15 | 31572539 | 31600650 | 6.06E−07 | −33.2466235 | Hypo-methylated | NEPC\|CRPC-Adeno DMRs |
| chr15 | 31620632 | 31622531 | 1.09E−05 | −23.3694134 | Hypo-methylated | NEPC\|CRPC-Adeno DMRs |
| chr15 | 32385694 | 32535167 | 0.001694183 | −33.57017544 | Hypo-methylated | NEPC\|CRPC-Adeno DMRs |
| chr15 | 34738238 | 34755823 | 0.001221669 | −23.69793602 | Hypo-methylated | NEPC\|CRPC-Adeno DMRs |
| chr15 | 39305560 | 39325707 | 0.001694183 | −23.76929825 | Hypo-methylated | NEPC\|CRPC-Adeno DMRs |
| chr15 | 40225618 | 40225853 | 0.000711935 | −26.66959064 | Hypo-methylated | NEPC\|CRPC-Adeno DMRs |
| chr15 | 40544411 | 40551526 | 1.31E−25 | −25.89625586 | Hypo-methylated | NEPC\|CRPC-Adeno DMRs |
| chr15 | 41210411 | 41211189 | 5.01E−05 | −40.20701754 | Hypo-methylated | NEPC\|CRPC-Adeno DMRs |
| chr15 | 41877483 | 41877790 | 1.90E−18 | −22.05305712 | Hypo-methylated | NEPC\|CRPC-Adeno DMRs |
| chr15 | 43502885 | 43503034 | 0.003327791 | −23.08355263 | Hypo-methylated | NEPC\|CRPC-Adeno DMRs |
| chr15 | 45327001 | 45375094 | 2.86E−05 | 20.28939628 | Hyper-methylated | NEPC\|CRPC-Adeno DMRs |
| chr15 | 52404242 | 52404929 | 3.83E−27 | −27.08735695 | Hypo-methylated | NEPC\|CRPC-Adeno DMRs |
| chr15 | 53081377 | 53081434 | 0.000277187 | 20.60436508 | Hyper-methylated | NEPC\|CRPC-Adeno DMRs |
| chr15 | 53097585 | 53097778 | 3.27E−07 | 22.72194457 | Hyper-methylated | NEPC\|CRPC-Adeno DMRs |
| chr15 | 55891230 | 55938721 | 0.000667554 | −22.54315789 | Hypo-methylated | NEPC\|CRPC-Adeno DMRs |
| chr15 | 58782494 | 58811992 | 1.97E−08 | −36.81179979 | Hypo-methylated | NEPC\|CRPC-Adeno DMRs |
| chr15 | 60287193 | 60288471 | 4.37E−10 | 28.36132519 | Hyper-methylated | NEPC\|CRPC-Adeno DMRs |
| chr15 | 62358751 | 62360466 | 5.66E−21 | −25.68828025 | Hypo-methylated | NEPC\|CRPC-Adeno DMRs |
| chr15 | 63334395 | 63334762 | 2.22E−20 | −27.5857457 | Hypo-methylated | NEPC\|CRPC-Adeno DMRs |
| chr15 | 63753498 | 63753733 | 0.0001248 | 20.09887218 | Hyper-methylated | NEPC\|CRPC-Adeno DMRs |
| chr15 | 65624830 | 65625248 | 0.00367696 | −24.11645401 | Hypo-methylated | NEPC\|CRPC-Adeno DMRs |
| chr15 | 65647425 | 65648343 | 9.42E−16 | −29.20798664 | Hypo-methylated | NEPC\|CRPC-Adeno DMRs |
| chr15 | 68098598 | 68098654 | 0.000832692 | −20.20935673 | Hypo-methylated | NEPC\|CRPC-Adeno DMRs |
| chr15 | 68631691 | 68631914 | 1.77E−05 | −21.57573881 | Hypo-methylated | NEPC\|CRPC-Adeno DMRs |
| chr15 | 68869879 | 68870394 | 0.000549237 | −21.92556647 | Hypo-methylated | NEPC\|CRPC-Adeno DMRs |
| chr15 | 69940620 | 69957148 | 0.000381376 | −35.303923 | Hypo-methylated | NEPC\|CRPC-Adeno DMRs |
| chr15 | 70519081 | 70519141 | 0.001694183 | −43.38664717 | Hypo-methylated | NEPC\|CRPC-Adeno DMRs |
| chr15 | 70544880 | 70544981 | 0.000549237 | −39.78345865 | Hypo-methylated | NEPC\|CRPC-Adeno DMRs |
| chr15 | 70707823 | 70708054 | 1.55E−06 | −39.73640351 | Hypo-methylated | NEPC\|CRPC-Adeno DMRs |
| chr15 | 70877436 | 70877832 | 8.70E−06 | 25.30467033 | Hyper-methylated | NEPC\|CRPC-Adeno DMRs |
| chr15 | 74266058 | 74266110 | 1.70E−05 | −33.85336134 | Hypo-methylated | NEPC\|CRPC-Adeno DMRs |
| chr15 | 74441833 | 74441902 | 0.001694183 | 43.27807018 | Hyper-methylated | NEPC\|CRPC-Adeno DMRs |
| chr15 | 74521145 | 74521366 | 0.00041315 | −49.06725146 | Hypo-methylated | NEPC\|CRPC-Adeno DMRs |
| chr15 | 75030067 | 75051090 | 2.46E−06 | −22.40754655 | Hypo-methylated | NEPC\|CRPC-Adeno DMRs |
| chr15 | 75108425 | 75108860 | 6.28E−07 | 20.06581384 | Hyper-methylated | NEPC\|CRPC-Adeno DMRs |
| chr15 | 76639476 | 76640054 | 2.79E−12 | 30.86904226 | Hyper-methylated | NEPC\|CRPC-Adeno DMRs |
| chr15 | 77923797 | 77924079 | 4.74E−05 | 32.41723761 | Hyper-methylated | NEPC\|CRPC-Adeno DMRs |
| chr15 | 79059018 | 79061261 | 0.001752695 | −22.36665438 | Hypo-methylated | NEPC\|CRPC-Adeno DMRs |
| chr15 | 79124278 | 79126387 | 1.43E−05 | −21.36977738 | Hypo-methylated | NEPC\|CRPC-Adeno DMRs |
| chr15 | 82336393 | 82336790 | 1.44E−11 | −31.77841777 | Hypo-methylated | NEPC\|CRPC-Adeno DMRs |
| chr15 | 83261457 | 83261553 | 0.000581135 | −33.59276316 | Hypo-methylated | NEPC\|CRPC-Adeno DMRs |
| chr15 | 83282560 | 83282647 | 0.001694183 | −28.54885478 | Hypo-methylated | NEPC\|CRPC-Adeno DMRs |
| chr15 | 83315699 | 83315950 | 0.005060933 | 21.51937522 | Hyper-methylated | NEPC\|CRPC-Adeno DMRs |
| chr15 | 83924232 | 83937882 | 7.58E−08 | −39.568059 | Hypo-methylated | NEPC\|CRPC-Adeno DMRs |
| chr15 | 84370721 | 84529633 | 3.84E−07 | −28.73719298 | Hypo-methylated | NEPC\|CRPC-Adeno DMRs |
| chr15 | 84547184 | 84672718 | 2.41E−06 | −26.36815215 | Hypo-methylated | NEPC\|CRPC-Adeno DMRs |
| chr15 | 86559748 | 86787108 | 3.64E−08 | −26.47819631 | Hypo-methylated | NEPC\|CRPC-Adeno DMRs |
| chr15 | 89367140 | 89390571 | 0.000111918 | −25.8840331 | Hypo-methylated | NEPC\|CRPC-Adeno DMRs |
| chr15 | 89392808 | 89430716 | 1.20E−07 | −21.87970908 | Hypo-methylated | NEPC\|CRPC-Adeno DMRs |
| chr15 | 89445844 | 89450655 | 0.004750844 | −21.15441077 | Hypo-methylated | NEPC\|CRPC-Adeno DMRs |
| chr15 | 89514733 | 89542703 | 0.000826274 | −20.704726 | Hypo-methylated | NEPC\|CRPC-Adeno DMRs |
| chr15 | 90006534 | 90030354 | 1.30E−05 | 23.13044362 | Hyper-methylated | NEPC\|CRPC-Adeno DMRs |
| chr15 | 90357585 | 90358061 | 5.71E−06 | 22.3002924 | Hyper-methylated | NEPC\|CRPC-Adeno DMRs |
| chr15 | 90543705 | 90543791 | 0.001654934 | −22.86861598 | Hypo-methylated | NEPC\|CRPC-Adeno DMRs |
| chr15 | 90837626 | 90844575 | 0.000586307 | −36.8609874 | Hypo-methylated | NEPC\|CRPC-Adeno DMRs |
| chr15 | 93010185 | 93043015 | 0.004222625 | −20.10112702 | Hypo-methylated | NEPC\|CRPC-Adeno DMRs |
| chr15 | 95388009 | 95388666 | 1.23E−08 | 21.60776521 | Hyper-methylated | NEPC\|CRPC-Adeno DMRs |
| chr15 | 96876804 | 96877682 | 1.75E−09 | 34.95865466 | Hyper-methylated | NEPC\|CRPC-Adeno DMRs |
| chr15 | 99092952 | 99128171 | 2.05E−08 | −29.02875167 | Hypo-methylated | NEPC\|CRPC-Adeno DMRs |
| chr15 | 99154066 | 99180015 | 3.88E−05 | −22.62285557 | Hypo-methylated | NEPC\|CRPC-Adeno DMRs |
| chr15 | 99194431 | 99194636 | 1.37E−07 | 36.07894737 | Hyper-methylated | NEPC\|CRPC-Adeno DMRs |
| chr15 | 99591062 | 99600352 | 0.003087144 | −23.52566219 | Hypo-methylated | NEPC\|CRPC-Adeno DMRs |
| chr15 | 99663731 | 99663937 | 0.001802623 | −20.5004386 | Hypo-methylated | NEPC\|CRPC-Adeno DMRs |
| chr15 | 100337390 | 100344594 | 0.00222033 | −28.01097459 | Hypo-methylated | NEPC\|CRPC-Adeno DMRs |
| chr15 | 100354296 | 100464678 | 0.002718013 | −32.02711362 | Hypo-methylated | NEPC\|CRPC-Adeno DMRs |
| chr15 | 100913597 | 100914023 | 1.00E−10 | 21.22424269 | Hyper-methylated | NEPC\|CRPC-Adeno DMRs |
| chr15 | 101344328 | 101385036 | 0.00324027 | −30.03501114 | Hypo-methylated | NEPC\|CRPC-Adeno DMRs |
| chr15 | 101394913 | 101398725 | 0.003113626 | −21.4205653 | Hypo-methylated | NEPC\|CRPC-Adeno DMRs |
| chr15 | 101661313 | 101661447 | 1.70E−05 | −43.03400376 | Hypo-methylated | NEPC\|CRPC-Adeno DMRs |
| chr15 | 101679706 | 101679973 | 0.000549237 | −34.17242511 | Hypo-methylated | NEPC\|CRPC-Adeno DMRs |
| chr15 | 101853489 | 101910850 | 4.45E−10 | −29.59507128 | Hypo-methylated | NEPC\|CRPC-Adeno DMRs |
| chr15 | 101915318 | 101921640 | 0.000176134 | −38.94205228 | Hypo-methylated | NEPC\|CRPC-Adeno DMRs |
| chr15 | 101934407 | 101936160 | 0.008985252 | −23.18588369 | Hypo-methylated | NEPC\|CRPC-Adeno DMRs |
| chr15 | 102042975 | 102073856 | 1.13E−07 | −26.46161405 | Hypo-methylated | NEPC\|CRPC-Adeno DMRs |
| chr15 | 102075075 | 102077597 | 9.43E−10 | −28.97655463 | Hypo-methylated | NEPC\|CRPC-Adeno DMRs |
| chr15 | 102101748 | 102144660 | 7.96E−07 | −30.18178855 | Hypo-methylated | NEPC\|CRPC-Adeno DMRs |
| chr15 | 102306000 | 102455350 | 0.002859601 | −20.81069143 | Hypo-methylated | NEPC\|CRPC-Adeno DMRs |
| chr16 | 113682 | 114458 | 0.000234574 | −26.78041705 | Hypo-methylated | NEPC\|CRPC-Adeno DMRs |
| chr16 | 210028 | 210106 | 0.001694183 | −22.91194653 | Hypo-methylated | NEPC\|CRPC-Adeno DMRs |
| chr16 | 323724 | 323869 | 0.001012154 | 20.95155747 | Hyper-methylated | NEPC\|CRPC-Adeno DMRs |
| chr16 | 422112 | 422265 | 2.46E−05 | −39.34084504 | Hypo-methylated | NEPC\|CRPC-Adeno DMRs |
| chr16 | 460250 | 460737 | 1.15E−08 | −27.60960047 | Hypo-methylated | NEPC\|CRPC-Adeno DMRs |
| chr16 | 633967 | 634126 | 9.74E−06 | −21.85380117 | Hypo-methylated | NEPC\|CRPC-Adeno DMRs |
| chr16 | 661074 | 661297 | 0.004025629 | −24.08011696 | Hypo-methylated | NEPC\|CRPC-Adeno DMRs |
| chr16 | 668173 | 671567 | 2.01E−08 | −26.57288788 | Hypo-methylated | NEPC\|CRPC-Adeno DMRs |
| chr16 | 787647 | 787982 | 8.67E−07 | 50.80386865 | Hyper-methylated | NEPC\|CRPC-Adeno DMRs |
| chr16 | 818242 | 819169 | 4.39E−05 | −28.70504386 | Hypo-methylated | NEPC\|CRPC-Adeno DMRs |
| chr16 | 832255 | 833018 | 3.06E−06 | −35.73884046 | Hypo-methylated | NEPC\|CRPC-Adeno DMRs |
| chr16 | 853454 | 855362 | 2.99E−06 | −27.78505613 | Hypo-methylated | NEPC\|CRPC-Adeno DMRs |
| chr16 | 871962 | 875955 | 9.14E−05 | −21.30733083 | Hypo-methylated | NEPC\|CRPC-Adeno DMRs |
| chr16 | 883655 | 893016 | 8.52E−07 | −24.15316922 | Hypo-methylated | NEPC\|CRPC-Adeno DMRs |
| chr16 | 902945 | 903125 | 3.42E−05 | −22.41526435 | Hypo-methylated | NEPC\|CRPC-Adeno DMRs |
| chr16 | 924435 | 924680 | 2.41E−07 | −30.63909523 | Hypo-methylated | NEPC\|CRPC-Adeno DMRs |
| chr16 | 963044 | 963184 | 0.000176134 | −27.80701754 | Hypo-methylated | NEPC\|CRPC-Adeno DMRs |
| chr16 | 967902 | 968683 | 0.000581135 | −24.55774854 | Hypo-methylated | NEPC\|CRPC-Adeno DMRs |
| chr16 | 1029085 | 1029193 | 0.009458442 | −20.59758772 | Hypo-methylated | NEPC\|CRPC-Adeno DMRs |
| chr16 | 1056659 | 1056952 | 7.68E−05 | −24.31265664 | Hypo-methylated | NEPC\|CRPC-Adeno DMRs |
| chr16 | 1101872 | 1104767 | 1.42E−06 | −28.31447667 | Hypo-methylated | NEPC\|CRPC-Adeno DMRs |
| chr16 | 1112649 | 1113198 | 0.00158762 | −20.97651179 | Hypo-methylated | NEPC\|CRPC-Adeno DMRs |
| chr16 | 1163076 | 1164302 | 1.29E−06 | −22.80018797 | Hypo-methylated | NEPC\|CRPC-Adeno DMRs |
| chr16 | 1164620 | 1164965 | 0.008187764 | −20.27443609 | Hypo-methylated | NEPC\|CRPC-Adeno DMRs |
| chr16 | 1181947 | 1183556 | 0.000756112 | −20.81161759 | Hypo-methylated | NEPC\|CRPC-Adeno DMRs |
| chr16 | 1194924 | 1195895 | 1.04E−09 | −37.14430289 | Hypo-methylated | NEPC\|CRPC-Adeno DMRs |
| chr16 | 1208467 | 1209004 | 1.02E−13 | −31.06076873 | Hypo-methylated | NEPC\|CRPC-Adeno DMRs |
| chr16 | 1228797 | 1228918 | 0.004583371 | −21.10120946 | Hypo-methylated | NEPC\|CRPC-Adeno DMRs |
| chr16 | 1259194 | 1259273 | 9.61E−06 | −23.89332958 | Hypo-methylated | NEPC\|CRPC-Adeno DMRs |
| chr16 | 1308022 | 1308153 | 0.001156603 | −20.94691858 | Hypo-methylated | NEPC\|CRPC-Adeno DMRs |
| chr16 | 1315857 | 1315926 | 0.000549237 | −36.50543024 | Hypo-methylated | NEPC\|CRPC-Adeno DMRs |
| chr16 | 1341592 | 1341834 | 1.55E−06 | −26.26803119 | Hypo-methylated | NEPC\|CRPC-Adeno DMRs |
| chr16 | 1428683 | 1428742 | 0.000176134 | 23.56798246 | Hyper-methylated | NEPC\|CRPC-Adeno DMRs |
| chr16 | 1475827 | 1477371 | 2.64E−09 | −43.18631916 | Hypo-methylated | NEPC\|CRPC-Adeno DMRs |
| chr16 | 1576464 | 1576669 | 5.50E−05 | −22.80563678 | Hypo-methylated | NEPC\|CRPC-Adeno DMRs |
| chr16 | 1921898 | 1922074 | 2.91E−07 | 25.25402417 | Hyper-methylated | NEPC\|CRPC-Adeno DMRs |
| chr16 | 2004755 | 2004922 | 0.000549237 | −32.44204261 | Hypo-methylated | NEPC\|CRPC-Adeno DMRs |
| chr16 | 2058089 | 2058391 | 0.000303685 | 23.06993177 | Hyper-methylated | NEPC\|CRPC-Adeno DMRs |
| chr16 | 2234860 | 2235549 | 8.10E−10 | −25.52513107 | Hypo-methylated | NEPC\|CRPC-Adeno DMRs |
| chr16 | 2531703 | 2533950 | 7.63E−12 | −45.71869471 | Hypo-methylated | NEPC\|CRPC-Adeno DMRs |
| chr16 | 2764280 | 2764394 | 0.001802623 | −28.72347536 | Hypo-methylated | NEPC\|CRPC-Adeno DMRs |
| chr16 | 2834976 | 2837658 | 6.58E−16 | −20.53630336 | Hypo-methylated | NEPC\|CRPC-Adeno DMRs |
| chr16 | 2865126 | 2865452 | 0.000153872 | −45.01432011 | Hypo-methylated | NEPC\|CRPC-Adeno DMRs |
| chr16 | 2880167 | 2880559 | 0.000233887 | 20.70445067 | Hyper-methylated | NEPC\|CRPC-Adeno DMRs |
| chr16 | 2956333 | 2956585 | 0.009458442 | −20.3079922 | Hypo-methylated | NEPC\|CRPC-Adeno DMRs |
| chr16 | 3003889 | 3004580 | 0.009285355 | −26.71361999 | Hypo-methylated | NEPC\|CRPC-Adeno DMRs |
| chr16 | 3140322 | 3140536 | 4.44E−08 | −26.03854333 | Hypo-methylated | NEPC\|CRPC-Adeno DMRs |
| chr16 | 3209157 | 3209326 | 1.83E−09 | 30.66894087 | Hyper-methylated | NEPC\|CRPC-Adeno DMRs |
| chr16 | 3228973 | 3229177 | 0.000953892 | −27.72235928 | Hypo-methylated | NEPC\|CRPC-Adeno DMRs |
| chr16 | 4661314 | 4661453 | 0.004750844 | 23.72717508 | Hyper-methylated | NEPC\|CRPC-Adeno DMRs |
| chr16 | 5185993 | 5235167 | 0.000156532 | −20.86065527 | Hypo-methylated | NEPC\|CRPC-Adeno DMRs |
| chr16 | 11348611 | 11348698 | 0.000176134 | −23.40789474 | Hypo-methylated | NEPC\|CRPC-Adeno DMRs |
| chr16 | 16244298 | 16244586 | 0.000168572 | −27.84398496 | Hypo-methylated | NEPC\|CRPC-Adeno DMRs |
| chr16 | 19113548 | 19113636 | 0.000176134 | −43.78571429 | Hypo-methylated | NEPC\|CRPC-Adeno DMRs |
| chr16 | 20135805 | 20291330 | 0.004652598 | −22.89454365 | Hypo-methylated | NEPC\|CRPC-Adeno DMRs |
| chr16 | 20430603 | 20641568 | 6.13E−06 | −28.62763889 | Hypo-methylated | NEPC\|CRPC-Adeno DMRs |
| chr16 | 20947948 | 20960045 | 0.002976353 | −21.75263158 | Hypo-methylated | NEPC\|CRPC-Adeno DMRs |
| chr16 | 22083989 | 22084058 | 0.000200845 | 26.64210526 | Hyper-methylated | NEPC\|CRPC-Adeno DMRs |
| chr16 | 23766378 | 23766426 | 2.91E−06 | 25.06892231 | Hyper-methylated | NEPC\|CRPC-Adeno DMRs |
| chr16 | 23847120 | 23848168 | 3.56E−08 | 27.88193139 | Hyper-methylated | NEPC\|CRPC-Adeno DMRs |
| chr16 | 25282189 | 25619765 | 3.70E−12 | −38.05398595 | Hypo-methylated | NEPC\|CRPC-Adeno DMRs |
| chr16 | 27078806 | 27095468 | 0.000235983 | −26.02061658 | Hypo-methylated | NEPC\|CRPC-Adeno DMRs |
| chr16 | 28333597 | 28334088 | 0.001549499 | 24.41285505 | Hyper-methylated | NEPC\|CRPC-Adeno DMRs |
| chr16 | 28873779 | 28874345 | 1.35E−05 | −21.01022659 | Hypo-methylated | NEPC\|CRPC-Adeno DMRs |
| chr16 | 29304319 | 29304728 | 5.50E−05 | −33.92898914 | Hypo-methylated | NEPC\|CRPC-Adeno DMRs |
| chr16 | 29328183 | 29328245 | 0.001694183 | −46.77192982 | Hypo-methylated | NEPC\|CRPC-Adeno DMRs |
| chr16 | 29624354 | 29625174 | 1.68E−21 | −20.03457361 | Hypo-methylated | NEPC\|CRPC-Adeno DMRs |
| chr16 | 29706242 | 29706608 | 0.000151874 | −31.72602719 | Hypo-methylated | NEPC\|CRPC-Adeno DMRs |
| chr16 | 34208831 | 34256502 | 0.000476227 | −21.97900185 | Hypo-methylated | NEPC\|CRPC-Adeno DMRs |
| chr16 | 34258912 | 34316427 | 3.43E−06 | −22.80407362 | Hypo-methylated | NEPC\|CRPC-Adeno DMRs |
| chr16 | 34379982 | 34380215 | 0.005899637 | −25.42 | Hypo-methylated | NEPC\|CRPC-Adeno DMRs |
| chr16 | 34407674 | 34408169 | 0.000286322 | −20.70916331 | Hypo-methylated | NEPC\|CRPC-Adeno DMRs |
| chr16 | 34456693 | 34472704 | 0.001274051 | −24.61945698 | Hypo-methylated | NEPC\|CRPC-Adeno DMRs |
| chr16 | 34490099 | 34561229 | 0.001012154 | −22.50215382 | Hypo-methylated | NEPC\|CRPC-Adeno DMRs |
| chr16 | 34578438 | 34622419 | 2.88E−06 | −22.03104094 | Hypo-methylated | NEPC\|CRPC-Adeno DMRs |
| chr16 | 34787178 | 34787394 | 0.000176134 | 23.33227124 | Hyper-methylated | NEPC\|CRPC-Adeno DMRs |
| chr16 | 49315604 | 49315759 | 0.000286322 | 31.0741956 | Hyper-methylated | NEPC\|CRPC-Adeno DMRs |
| chr16 | 49315841 | 49316610 | 1.19E−23 | 29.34917625 | Hyper-methylated | NEPC\|CRPC-Adeno DMRs |
| chr16 | 49317120 | 49317299 | 0.000176134 | 27.53905843 | Hyper-methylated | NEPC\|CRPC-Adeno DMRs |
| chr16 | 49475473 | 49486702 | 0.000194541 | −22.7348194 | Hypo-methylated | NEPC\|CRPC-Adeno DMRs |
| chr16 | 49541930 | 49559604 | 9.63E−14 | −33.78854036 | Hypo-methylated | NEPC\|CRPC-Adeno DMRs |
| chr16 | 49737799 | 49749375 | 8.98E−06 | −23.89931517 | Hypo-methylated | NEPC\|CRPC-Adeno DMRs |
| chr16 | 50637405 | 50647255 | 5.75E−05 | 21.7925559 | Hyper-methylated | NEPC\|CRPC-Adeno DMRs |
| chr16 | 51186522 | 51188707 | 8.31E−10 | 31.38498452 | Hyper-methylated | NEPC\|CRPC-Adeno DMRs |
| chr16 | 51305036 | 51426357 | 0.000154411 | 23.74670232 | Hyper-methylated | NEPC\|CRPC-Adeno DMRs |
| chr16 | 55015881 | 55021257 | 0.000194541 | −22.14736842 | Hypo-methylated | NEPC\|CRPC-Adeno DMRs |
| chr16 | 55090618 | 55090848 | 1.30E−09 | 44.20583649 | Hyper-methylated | NEPC\|CRPC-Adeno DMRs |
| chr16 | 55358693 | 55358725 | 5.50E−05 | 21.97309942 | Hyper-methylated | NEPC\|CRPC-Adeno DMRs |
| chr16 | 56345858 | 56346113 | 0.000549237 | −58.39924812 | Hypo-methylated | NEPC\|CRPC-Adeno DMRs |
| chr16 | 58787856 | 58868597 | 0.001552631 | −23.50858206 | Hypo-methylated | NEPC\|CRPC-Adeno DMRs |
| chr16 | 65093517 | 65147114 | 0.000129108 | −23.14791267 | Hypo-methylated | NEPC\|CRPC-Adeno DMRs |
| chr16 | 65157596 | 65157663 | 4.24E−06 | 20.87510443 | Hyper-methylated | NEPC\|CRPC-Adeno DMRs |
| chr16 | 65431969 | 65732199 | 5.88E−09 | −25.19268547 | Hypo-methylated | NEPC\|CRPC-Adeno DMRs |
| chr16 | 66091780 | 66091933 | 0.001802623 | −29.72769722 | Hypo-methylated | NEPC\|CRPC-Adeno DMRs |
| chr16 | 66550312 | 66556659 | 0.005737741 | 26.29181287 | Hyper-methylated | NEPC\|CRPC-Adeno DMRs |
| chr16 | 69348684 | 69348993 | 0.000214284 | 22.9235589 | Hyper-methylated | NEPC\|CRPC-Adeno DMRs |
| chr16 | 69776044 | 69776370 | 0.008985252 | 28.15278938 | Hyper-methylated | NEPC\|CRPC-Adeno DMRs |
| chr16 | 69999395 | 69999434 | 0.001694183 | −21.5489418 | Hypo-methylated | NEPC\|CRPC-Adeno DMRs |
| chr16 | 70680625 | 70680999 | 0.000549237 | −56.82587421 | Hypo-methylated | NEPC\|CRPC-Adeno DMRs |
| chr16 | 70713796 | 70715428 | 2.94E−06 | 21.51629739 | Hyper-methylated | NEPC\|CRPC-Adeno DMRs |
| chr16 | 70771008 | 70772688 | 0.002751359 | 22.66231048 | Hyper-methylated | NEPC\|CRPC-Adeno DMRs |
| chr16 | 71475581 | 71475744 | 4.87E−07 | 24.95883607 | Hyper-methylated | NEPC\|CRPC-Adeno DMRs |
| chr16 | 71491322 | 71491391 | 0.000549237 | 20.54511278 | Hyper-methylated | NEPC\|CRPC-Adeno DMRs |
| chr16 | 72821150 | 72821200 | 0.001694183 | 40.92766885 | Hyper-methylated | NEPC\|CRPC-Adeno DMRs |
| chr16 | 72955745 | 72974666 | 0.000398142 | −24.17671451 | Hypo-methylated | NEPC\|CRPC-Adeno DMRs |
| chr16 | 75034035 | 75034591 | 0.001151409 | −27.43166228 | Hypo-methylated | NEPC\|CRPC-Adeno DMRs |
| chr16 | 76668924 | 76669090 | 0.000176134 | −36.68219246 | Hypo-methylated | NEPC\|CRPC-Adeno DMRs |
| chr16 | 79007120 | 79007227 | 0.00040075 | −42.07395003 | Hypo-methylated | NEPC\|CRPC-Adeno DMRs |
| chr16 | 79027562 | 79030799 | 5.17E−06 | −41.74704944 | Hypo-methylated | NEPC\|CRPC-Adeno DMRs |
| chr16 | 80698286 | 80714829 | 0.003113626 | −24.75555556 | Hypo-methylated | NEPC\|CRPC-Adeno DMRs |
| chr16 | 80915529 | 80950178 | 0.000101637 | −25.41132898 | Hypo-methylated | NEPC\|CRPC-Adeno DMRs |
| chr16 | 81042069 | 81042229 | 0.001065261 | −27.52716049 | Hypo-methylated | NEPC\|CRPC-Adeno DMRs |
| chr16 | 81465376 | 81465542 | 5.17E−06 | −50.40151515 | Hypo-methylated | NEPC\|CRPC-Adeno DMRs |
| chr16 | 81549231 | 81549956 | 1.37E−07 | −34.34547152 | Hypo-methylated | NEPC\|CRPC-Adeno DMRs |
| chr16 | 81587130 | 81587186 | 0.000549237 | −29.55079365 | Hypo-methylated | NEPC\|CRPC-Adeno DMRs |
| chr16 | 81672526 | 81672686 | 0.001065261 | 22.44953833 | Hyper-methylated | NEPC\|CRPC-Adeno DMRs |
| chr16 | 81685877 | 81702223 | 9.02E−06 | −45.37774221 | Hypo-methylated | NEPC\|CRPC-Adeno DMRs |
| chr16 | 81739782 | 81741668 | 0.001446886 | −21.12238013 | Hypo-methylated | NEPC\|CRPC-Adeno DMRs |
| chr16 | 82660676 | 82660728 | 0.000176134 | 24.54775219 | Hyper-methylated | NEPC\|CRPC-Adeno DMRs |
| chr16 | 83765681 | 83766983 | 0.001802623 | −32.39482038 | Hypo-methylated | NEPC\|CRPC-Adeno DMRs |
| chr16 | 83974425 | 83975062 | 1.60E−06 | −37.41336032 | Hypo-methylated | NEPC\|CRPC-Adeno DMRs |
| chr16 | 84074769 | 84077082 | 6.49E−05 | −41.20839676 | Hypo-methylated | NEPC\|CRPC-Adeno DMRs |
| chr16 | 84194396 | 84204832 | 3.63E−05 | −23.79258686 | Hypo-methylated | NEPC\|CRPC-Adeno DMRs |
| chr16 | 84551016 | 84553586 | 0.000700082 | −44.61167446 | Hypo-methylated | NEPC\|CRPC-Adeno DMRs |
| chr16 | 84599812 | 84600052 | 0.000200845 | −35.09385191 | Hypo-methylated | NEPC\|CRPC-Adeno DMRs |
| chr16 | 84609911 | 84617818 | 4.65E−07 | −37.6535152 | Hypo-methylated | NEPC\|CRPC-Adeno DMRs |
| chr16 | 84840191 | 84840468 | 1.55E−06 | −34.60175439 | Hypo-methylated | NEPC\|CRPC-Adeno DMRs |
| chr16 | 85320513 | 85320775 | 2.78E−10 | −36.95352416 | Hypo-methylated | NEPC\|CRPC-Adeno DMRs |
| chr16 | 85337122 | 85337367 | 0.001694183 | 30.11031746 | Hyper-methylated | NEPC\|CRPC-Adeno DMRs |
| chr16 | 85393998 | 85395781 | 1.36E−08 | −52.11873705 | Hypo-methylated | NEPC\|CRPC-Adeno DMRs |
| chr16 | 85496576 | 85514744 | 1.20E−08 | −33.86475636 | Hypo-methylated | NEPC\|CRPC-Adeno DMRs |
| chr16 | 85596147 | 85596236 | 0.001694183 | −60.25536062 | Hypo-methylated | NEPC\|CRPC-Adeno DMRs |
| chr16 | 85627523 | 85627767 | 0.001274051 | −49.75840213 | Hypo-methylated | NEPC\|CRPC-Adeno DMRs |
| chr16 | 85635743 | 85636514 | 0.000113947 | −34.14437135 | Hypo-methylated | NEPC\|CRPC-Adeno DMRs |
| chr16 | 85653107 | 85662013 | 3.84E−08 | −29.57493665 | Hypo-methylated | NEPC\|CRPC-Adeno DMRs |
| chr16 | 85966695 | 85979154 | 2.57E−08 | −32.05703678 | Hypo-methylated | NEPC\|CRPC-Adeno DMRs |
| chr16 | 86600284 | 86600932 | 4.04E−17 | 27.25250778 | Hyper-methylated | NEPC\|CRPC-Adeno DMRs |
| chr16 | 86601002 | 86601457 | 4.56E−09 | 25.9594233 | Hyper-methylated | NEPC\|CRPC-Adeno DMRs |
| chr16 | 86601587 | 86601666 | 1.37E−07 | 24.17940685 | Hyper-methylated | NEPC\|CRPC-Adeno DMRs |
| chr16 | 86601856 | 86601911 | 0.000118331 | 25.51816239 | Hyper-methylated | NEPC\|CRPC-Adeno DMRs |
| chr16 | 86602118 | 86602229 | 4.24E−06 | 25.39592471 | Hyper-methylated | NEPC\|CRPC-Adeno DMRs |
| chr16 | 86612819 | 86612851 | 0.000176134 | 21.93407999 | Hyper-methylated | NEPC\|CRPC-Adeno DMRs |
| chr16 | 87524165 | 87525417 | 4.73E−08 | −24.10266637 | Hypo-methylated | NEPC\|CRPC-Adeno DMRs |
| chr16 | 87722353 | 87722683 | 0.000379987 | −21.54571736 | Hypo-methylated | NEPC\|CRPC-Adeno DMRs |
| chr16 | 87738833 | 87739716 | 0.004216708 | −23.28031008 | Hypo-methylated | NEPC\|CRPC-Adeno DMRs |
| chr16 | 87757047 | 87757111 | 0.003525992 | −35.2417154 | Hypo-methylated | NEPC\|CRPC-Adeno DMRs |
| chr16 | 87840158 | 87840512 | 5.99E−05 | −20.55639098 | Hypo-methylated | NEPC\|CRPC-Adeno DMRs |
| chr16 | 87902373 | 87903252 | 3.41E−20 | −20.01363372 | Hypo-methylated | NEPC\|CRPC-Adeno DMRs |
| chr16 | 88017821 | 88017887 | 0.000549237 | 37.3700919 | Hyper-methylated | NEPC\|CRPC-Adeno DMRs |
| chr16 | 88053037 | 88053146 | 0.001694183 | 23.75341131 | Hyper-methylated | NEPC\|CRPC-Adeno DMRs |
| chr16 | 88096362 | 88099447 | 0.00010581 | 20.89598997 | Hyper-methylated | NEPC\|CRPC-Adeno DMRs |
| chr16 | 88134817 | 88135083 | 0.009458442 | −25.72417154 | Hypo-methylated | NEPC\|CRPC-Adeno DMRs |
| chr16 | 88144486 | 88161666 | 4.58E−08 | −21.74832719 | Hypo-methylated | NEPC\|CRPC-Adeno DMRs |
| chr16 | 88166151 | 88169176 | 0.000324528 | −22.32145468 | Hypo-methylated | NEPC\|CRPC-Adeno DMRs |
| chr16 | 88184118 | 88184820 | 0.000155879 | −26.75048733 | Hypo-methylated | NEPC\|CRPC-Adeno DMRs |
| chr16 | 88228127 | 88228483 | 0.00678631 | −31.29760766 | Hypo-methylated | NEPC\|CRPC-Adeno DMRs |
| chr16 | 88260875 | 88266663 | 1.55E−06 | −28.58778643 | Hypo-methylated | NEPC\|CRPC-Adeno DMRs |
| chr16 | 88294661 | 88316692 | 7.10E−08 | −28.4773006 | Hypo-methylated | NEPC\|CRPC-Adeno DMRs |
| chr16 | 88452947 | 88453817 | 0.00013534 | −24.52246466 | Hypo-methylated | NEPC\|CRPC-Adeno DMRs |
| chr16 | 88606149 | 88608168 | 2.28E−05 | −25.05968045 | Hypo-methylated | NEPC\|CRPC-Adeno DMRs |
| chr16 | 88742761 | 88743044 | 0.000176134 | −44.55471169 | Hypo-methylated | NEPC\|CRPC-Adeno DMRs |
| chr16 | 88800103 | 88800909 | 2.94E−06 | −24.70305973 | Hypo-methylated | NEPC\|CRPC-Adeno DMRs |
| chr16 | 88879949 | 88883094 | 0.000921835 | −29.20717513 | Hypo-methylated | NEPC\|CRPC-Adeno DMRs |
| chr16 | 88953682 | 88953888 | 0.001532655 | −27.42852792 | Hypo-methylated | NEPC\|CRPC-Adeno DMRs |
| chr16 | 88990394 | 88992139 | 9.75E−08 | −40.39645216 | Hypo-methylated | NEPC\|CRPC-Adeno DMRs |
| chr16 | 89055721 | 89055932 | 1.55E−06 | −45.43057644 | Hypo-methylated | NEPC\|CRPC-Adeno DMRs |
| chr16 | 89202582 | 89202990 | 2.76E−05 | −22.65460526 | Hypo-methylated | NEPC\|CRPC-Adeno DMRs |
| chr16 | 89227434 | 89229027 | 0.003484081 | −22.30962824 | Hypo-methylated | NEPC\|CRPC-Adeno DMRs |
| chr16 | 89241586 | 89242638 | 5.23E−05 | −28.61555878 | Hypo-methylated | NEPC\|CRPC-Adeno DMRs |
| chr16 | 89266568 | 89266808 | 0.000484821 | −26.39107143 | Hypo-methylated | NEPC\|CRPC-Adeno DMRs |
| chr16 | 89651682 | 89651863 | 0.000324528 | −39.53326023 | Hypo-methylated | NEPC\|CRPC-Adeno DMRs |
| chr16 | 89697640 | 89698943 | 0.005992671 | −23.59464286 | Hypo-methylated | NEPC\|CRPC-Adeno DMRs |
| chr16 | 89764190 | 89764332 | 1.77E−05 | −38.12559809 | Hypo-methylated | NEPC\|CRPC-Adeno DMRs |
| chr16 | 89972593 | 89972865 | 6.03E−06 | −27.32763158 | Hypo-methylated | NEPC\|CRPC-Adeno DMRs |
| chr16 | 90066682 | 90068394 | 0.001694183 | 23.26970551 | Hyper-methylated | NEPC\|CRPC-Adeno DMRs |
| chr16 | 90148922 | 90148997 | 0.001715076 | −23.85760234 | Hypo-methylated | NEPC\|CRPC-Adeno DMRs |
| chr17 | 155004 | 155155 | 0.002094266 | −29.52155388 | Hypo-methylated | NEPC\|CRPC-Adeno DMRs |
| chr17 | 216813 | 216897 | 1.55E−06 | −43.8495614 | Hypo-methylated | NEPC\|CRPC-Adeno DMRs |
| chr17 | 291029 | 292905 | 1.87E−09 | −23.16203008 | Hypo-methylated | NEPC\|CRPC-Adeno DMRs |
| chr17 | 400400 | 407261 | 0.00040075 | −21.76733738 | Hypo-methylated | NEPC\|CRPC-Adeno DMRs |
| chr17 | 707821 | 708522 | 2.51E−06 | −34.75465418 | Hypo-methylated | NEPC\|CRPC-Adeno DMRs |
| chr17 | 729503 | 732556 | 0.000392904 | −20.73004948 | Hypo-methylated | NEPC\|CRPC-Adeno DMRs |
| chr17 | 915194 | 915963 | 5.69E−17 | −43.95834833 | Hypo-methylated | NEPC\|CRPC-Adeno DMRs |
| chr17 | 1011493 | 1011548 | 0.001694183 | −21.81810429 | Hypo-methylated | NEPC\|CRPC-Adeno DMRs |
| chr17 | 1026731 | 1027752 | 0.000300035 | −29.3187794 | Hypo-methylated | NEPC\|CRPC-Adeno DMRs |
| chr17 | 1174036 | 1174134 | 7.87E−11 | 22.93517544 | Hyper-methylated | NEPC\|CRPC-Adeno DMRs |
| chr17 | 1184078 | 1184313 | 3.94E−05 | −21.50020846 | Hypo-methylated | NEPC\|CRPC-Adeno DMRs |
| chr17 | 1509924 | 1510019 | 0.000303685 | −27.42280702 | Hypo-methylated | NEPC\|CRPC-Adeno DMRs |
| chr17 | 1614142 | 1618107 | 0.000755213 | 25.45276316 | Hyper-methylated | NEPC\|CRPC-Adeno DMRs |
| chr17 | 1658766 | 1659342 | 3.15E−05 | −38.53315789 | Hypo-methylated | NEPC\|CRPC-Adeno DMRs |
| chr17 | 1819866 | 1822567 | 5.76E−06 | −33.77989934 | Hypo-methylated | NEPC\|CRPC-Adeno DMRs |
| chr17 | 1861788 | 1864443 | 2.01E−06 | −37.03054163 | Hypo-methylated | NEPC\|CRPC-Adeno DMRs |
| chr17 | 2207832 | 2207954 | 0.000200845 | −33.66180451 | Hypo-methylated | NEPC\|CRPC-Adeno DMRs |
| chr17 | 2262937 | 2263058 | 1.30E−06 | −28.31117974 | Hypo-methylated | NEPC\|CRPC-Adeno DMRs |
| chr17 | 2278817 | 2279116 | 0.000176134 | 20.37469846 | Hyper-methylated | NEPC\|CRPC-Adeno DMRs |
| chr17 | 2806262 | 2807630 | 0.002989545 | −20.93184476 | Hypo-methylated | NEPC\|CRPC-Adeno DMRs |
| chr17 | 3549305 | 3549546 | 1.70E−05 | −33.4480805 | Hypo-methylated | NEPC\|CRPC-Adeno DMRs |
| chr17 | 3588767 | 3591497 | 0.001012154 | −25.08514142 | Hypo-methylated | NEPC\|CRPC-Adeno DMRs |
| chr17 | 3652671 | 3652708 | 0.000953892 | −23.96317617 | Hypo-methylated | NEPC\|CRPC-Adeno DMRs |
| chr17 | 3858421 | 3858592 | 0.000153872 | −28.16491228 | Hypo-methylated | NEPC\|CRPC-Adeno DMRs |
| chr17 | 3907313 | 3907538 | 8.34E−06 | 39.30190058 | Hyper-methylated | NEPC\|CRPC-Adeno DMRs |
| chr17 | 4074101 | 4074194 | 0.000176134 | −20.84356725 | Hypo-methylated | NEPC\|CRPC-Adeno DMRs |
| chr17 | 4478978 | 4479211 | 0.007673974 | −25.8215812 | Hypo-methylated | NEPC\|CRPC-Adeno DMRs |
| chr17 | 4502955 | 4503992 | 6.29E−06 | −39.48704733 | Hypo-methylated | NEPC\|CRPC-Adeno DMRs |
| chr17 | 5015907 | 5016031 | 0.000176134 | −35.05637792 | Hypo-methylated | NEPC\|CRPC-Adeno DMRs |
| chr17 | 5026278 | 5033539 | 3.73E−10 | −23.35926354 | Hypo-methylated | NEPC\|CRPC-Adeno DMRs |
| chr17 | 5740692 | 5772485 | 0.000953892 | 26.91825397 | Hyper-methylated | NEPC\|CRPC-Adeno DMRs |
| chr17 | 6606244 | 6610641 | 0.000286322 | −23.66867168 | Hypo-methylated | NEPC\|CRPC-Adeno DMRs |
| chr17 | 7010399 | 7010665 | 0.000176134 | −52.91644737 | Hypo-methylated | NEPC\|CRPC-Adeno DMRs |
| chr17 | 7041409 | 7043582 | 0.000200845 | −37.95918755 | Hypo-methylated | NEPC\|CRPC-Adeno DMRs |
| chr17 | 7349953 | 7350440 | 3.04E−05 | 22.63500223 | Hyper-methylated | NEPC\|CRPC-Adeno DMRs |
| chr17 | 7942503 | 7942938 | 1.35E−09 | 22.86024436 | Hyper-methylated | NEPC\|CRPC-Adeno DMRs |
| chr17 | 8053368 | 8054763 | 0.0001438 | 32.68809841 | Hyper-methylated | NEPC\|CRPC-Adeno DMRs |
| chr17 | 8702445 | 8702752 | 3.09E−12 | −27.00337275 | Hypo-methylated | NEPC\|CRPC-Adeno DMRs |
| chr17 | 8931504 | 8968415 | 0.00055492 | −26.48604524 | Hypo-methylated | NEPC\|CRPC-Adeno DMRs |
| chr17 | 9048263 | 9054773 | 1.27E−05 | −36.22605994 | Hypo-methylated | NEPC\|CRPC-Adeno DMRs |
| chr17 | 9960630 | 10100003 | 1.46E−18 | −36.79670646 | Hypo-methylated | NEPC\|CRPC-Adeno DMRs |
| chr17 | 10110814 | 10220689 | 4.02E−07 | −36.82443504 | Hypo-methylated | NEPC\|CRPC-Adeno DMRs |
| chr17 | 10230613 | 10269859 | 7.07E−07 | 35.94446794 | Hypo-methylated | NEPC\|CRPC-Adeno DMRs |
| chr17 | 10528325 | 10528450 | 0.000953892 | −32.56184211 | Hypo-methylated | NEPC\|CRPC-Adeno DMRs |
| chrl7 | 10535221 | 10559324 | 0.000392904 | −24.73589744 | Hypo-methylated | NEPC\|CRPC-Adeno DMRs |
| chr17 | 10706731 | 10761061 | 6.29E−07 | −44.01210584 | Hypo-methylated | NEPC\|CRPC-Adeno DMRs |
| chr17 | 10782976 | 11096605 | 6.22E−05 | −25.59527198 | Hypo-methylated | NEPC\|CRPC-Adeno DMRs |
| chr17 | 11228359 | 11490071 | 5.29E−13 | −23.84131368 | Hypo-methylated | NEPC\|CRPC-Adeno DMRs |
| chr17 | 11520809 | 11679161 | 2.68E−15 | −28.80589106 | Hypo-methylated | NEPC\|CRPC-Adeno DMRs |
| chr17 | 11687648 | 11866643 | 5.92E−08 | −23.26462034 | Hypo-methylated | NEPC\|CRPC-Adeno DMRs |
| chr17 | 12151995 | 12205432 | 1.55E−06 | −32.83114035 | Hypo-methylated | NEPC\|CRPC-Adeno DMRs |
| chr17 | 12615473 | 12622349 | 0.005737741 | −28.74147173 | Hypo-methylated | NEPC\|CRPC-Adeno DMRs |
| chr17 | 12650265 | 12681844 | 1.82E−05 | −27.51982878 | Hypo-methylated | NEPC\|CRPC-Adeno DMRs |
| chr17 | 14184762 | 14198441 | 1.70E−05 | 21.21725146 | Hyper-methylated | NEPC\|CRPC-Adeno DMRs |
| chr17 | 14201910 | 14202097 | 6.48E−07 | 23.07017544 | Hyper-methylated | NEPC\|CRPC-Adeno DMRs |
| chr17 | 15164917 | 15166502 | 1.94E−06 | 23.84690894 | Hyper-methylated | NEPC\|CRPC-Adeno DMRs |
| chr17 | 15652986 | 15653116 | 1.09E−08 | 23.14568427 | Hyper-methylated | NEPC\|CRPC-Adeno DMRs |
| chr17 | 15847739 | 15847786 | 0.005737741 | 21.34015595 | Hyper-methylated | NEPC\|CRPC-Adeno DMRs |
| chr17 | 16373648 | 16373942 | 1.55E−06 | −41.12807018 | Hypo-methylated | NEPC\|CRPC-Adeno DMRs |
| chr17 | 16908675 | 16911402 | 0.005737741 | −22.02797619 | Hypo-methylated | NEPC\|CRPC-Adeno DMRs |
| chr17 | 16932252 | 16933471 | 0.009458442 | −27.97642544 | Hypo-methylated | NEPC\|CRPC-Adeno DMRs |
| chr17 | 17258920 | 17259771 | 0.000549237 | −34.36915082 | Hypo-methylated | NEPC\|CRPC-Adeno DMRs |
| chr17 | 17345719 | 17345760 | 0.001694183 | −38.85972222 | Hypo-methylated | NEPC\|CRPC-Adeno DMRs |
| chr17 | 17649212 | 17652682 | 5.79E−07 | −37.03412281 | Hypo-methylated | NEPC\|CRPC-Adeno DMRs |
| chr17 | 17815463 | 17815842 | 0.004750844 | −26.75044497 | Hypo-methylated | NEPC\|CRPC-Adeno DMRs |
| chr17 | 18026466 | 18026639 | 0.004906972 | −22.08220098 | Hypo-methylated | NEPC\|CRPC-Adeno DMRs |
| chr17 | 18150147 | 18150579 | 2.20E−08 | −33.40402047 | Hypo-methylated | NEPC\|CRPC-Adeno DMRs |
| chr17 | 18870409 | 18870875 | 0.001012154 | −27.59022556 | Hypo-methylated | NEPC\|CRPC-Adeno DMRs |
| chr17 | 19282282 | 19282596 | 1.22E−07 | −23.26481051 | Hypo-methylated | NEPC\|CRPC-Adeno DMRs |
| chr17 | 21214022 | 21214072 | 0.009458442 | −23.65877193 | Hypo-methylated | NEPC\|CRPC-Adeno DMRs |
| chr17 | 21241637 | 21253974 | 0.000219024 | −29.63203671 | Hypo-methylated | NEPC\|CRPC-Adeno DMRs |
| chr17 | 21308403 | 21320519 | 1.24E−09 | −22.12445853 | Hypo-methylated | NEPC\|CRPC-Adeno DMRs |
| chr17 | 21476839 | 21477072 | 0.001694183 | −29.03220551 | Hypo-methylated | NEPC\|CRPC-Adeno DMRs |
| chr17 | 21561545 | 21561832 | 0.001229862 | −20.61754386 | Hypo-methylated | NEPC\|CRPC-Adeno DMRs |
| chr17 | 21934869 | 21953670 | 0.000102266 | −25.24649608 | Hypo-methylated | NEPC\|CRPC-Adeno DMRs |
| chr17 | 21953785 | 21954046 | 0.000549777 | −24.70929027 | Hypo-methylated | NEPC\|CRPC-Adeno DMRs |
| chr17 | 22203643 | 25282926 | 7.95E−06 | −25.59312853 | Hypo-methylated | NEPC\|CRPC-Adeno DMRs |
| chr17 | 25289816 | 25289898 | 1.17E−08 | 32.27715121 | Hyper-methylated | NEPC\|CRPC-Adeno DMRs |
| chr17 | 25480583 | 25480961 | 0.002976353 | −28.22789818 | Hypo-methylated | NEPC\|CRPC-Adeno DMRs |
| chr17 | 26732104 | 26732177 | 0.001694183 | −20.42631579 | Hypo-methylated | NEPC\|CRPC-Adeno DMRs |
| chrl7 | 27292056 | 27296952 | 0.009285355 | 27.38902649 | Hyper-methylated | NEPC\|CRPC-Adeno DMRs |
| chr17 | 27326507 | 27326575 | 0.001694183 | −30.85087719 | Hypo-methylated | NEPC\|CRPC-Adeno DMRs |
| chr17 | 27332339 | 27332711 | 2.46E−15 | −21.55506386 | Hypo-methylated | NEPC\|CRPC-Adeno DMRs |
| chr17 | 28299554 | 28299747 | 2.76E−05 | −60.86517028 | Hypo-methylated | NEPC\|CRPC-Adeno DMRs |
| chr17 | 28885977 | 28886196 | 0.000153872 | −24.49707602 | Hypo-methylated | NEPC\|CRPC-Adeno DMRs |
| chr17 | 29886869 | 29888729 | 2.86E−13 | 23.65564264 | Hyper-methylated | NEPC\|CRPC-Adeno DMRs |
| chr17 | 30814995 | 30816457 | 2.48E−05 | −28.88156666 | Hypo-methylated | NEPC\|CRPC-Adeno DMRs |
| chr17 | 31011187 | 31011254 | 0.000828392 | −26.99342105 | Hypo-methylated | NEPC\|CRPC-Adeno DMRs |
| chr17 | 33324725 | 33330630 | 0.002174767 | 21.78997569 | Hyper-methylated | NEPC\|CRPC-Adeno DMRs |
| chr17 | 33776533 | 33776872 | 2.19E−09 | 29.03399359 | Hyper-methylated | NEPC\|CRPC-Adeno DMRs |
| chr17 | 34068350 | 34068658 | 5.59E−10 | −30.16247515 | Hypo-methylated | NEPC\|CRPC-Adeno DMRs |
| chr17 | 35017720 | 35017946 | 0.000224358 | −35.25577485 | Hypo-methylated | NEPC\|CRPC-Adeno DMRs |
| chr17 | 36286578 | 36371581 | 2.46E−05 | −28.06637989 | Hypo-methylated | NEPC\|CRPC-Adeno DMRs |
| chr17 | 36666143 | 36667137 | 1.72E−33 | −24.4491434 | Hypo-methylated | NEPC\|CRPC-Adeno DMRs |
| chr17 | 36692433 | 36696116 | 1.70E−05 | −42.31842105 | Hypo-methylated | NEPC\|CRPC-Adeno DMRs |
| chr17 | 36813715 | 36817440 | 0.003063309 | −26.951417 | Hypo-methylated | NEPC\|CRPC-Adeno DMRs |
| chr17 | 38769073 | 38769292 | 0.004750844 | −21.5380117 | Hypo-methylated | NEPC\|CRPC-Adeno DMRs |
| chr17 | 41607883 | 41608483 | 1.55E−06 | −47.48927005 | Hypo-methylated | NEPC\|CRPC-Adeno DMRs |
| chr17 | 42081920 | 42083794 | 3.91E−24 | −22.77831257 | Hypo-methylated | NEPC\|CRPC-Adeno DMRs |
| chr17 | 42733408 | 42733893 | 0.003652661 | 23.54033521 | Hyper-methylated | NEPC\|CRPC-Adeno DMRs |
| chr17 | 42734844 | 42734898 | 4.74E−05 | −20.90951811 | Hypo-methylated | NEPC\|CRPC-Adeno DMRs |
| chr17 | 43037212 | 43037655 | 2.70E−07 | 24.53241735 | Hyper-methylated | NEPC\|CRPC-Adeno DMRs |
| chr17 | 43973529 | 43973730 | 2.64E−09 | −23.34772995 | Hypo-methylated | NEPC\|CRPC-Adeno DMRs |
| chr17 | 44836293 | 44836427 | 0.005737741 | 30.19705309 | Hyper-methylated | NEPC\|CRPC-Adeno DMRs |
| chr17 | 45501212 | 45501632 | 5.65E−10 | −22.65046763 | Hypo-methylated | NEPC\|CRPC-Adeno DMRs |
| chr17 | 47075095 | 47075887 | 7.01E−05 | 25.53349893 | Hyper-methylated | NEPC\|CRPC-Adeno DMRs |
| chr17 | 48188524 | 48190385 | 3.70E−06 | −24.8640186 | Hypo-methylated | NEPC\|CRPC-Adeno DMRs |
| chr17 | 48596375 | 48596437 | 0.001694183 | −32.83442982 | Hypo-methylated | NEPC\|CRPC-Adeno DMRs |
| chr17 | 48858799 | 48858849 | 0.000176134 | 24.45476974 | Hyper-methylated | NEPC\|CRPC-Adeno DMRs |
| chr17 | 55371596 | 55391244 | 7.16E−05 | −27.45328897 | Hypo-methylated | NEPC\|CRPC-Adeno DMRs |
| chr17 | 55632533 | 55642757 | 0.000832692 | −29.69913194 | Hypo-methylated | NEPC\|CRPC-Adeno DMRs |
| chr17 | 55970829 | 56004262 | 0.000176134 | −28.6679605 | Hypo-methylated | NEPC\|CRPC-Adeno DMRs |
| chr17 | 57642658 | 57642805 | 0.00357488 | 20.63512146 | Hyper-methylated | NEPC\|CRPC-Adeno DMRs |
| chr17 | 60217446 | 60217678 | 4.74E−05 | −26.4645933 | Hypo-methylated | NEPC\|CRPC-Adeno DMRs |
| chr17 | 60767466 | 60767595 | 0.001694183 | −29.67098301 | Hypo-methylated | NEPC\|CRPC-Adeno DMRs |
| chr17 | 60821673 | 60827531 | 0.00222033 | −24.91475318 | Hypo-methylated | NEPC\|CRPC-Adeno DMRs |
| chr17 | 61926254 | 61926882 | 4.21E−09 | 27.03917345 | Hyper-methylated | NEPC\|CRPC-Adeno DMRs |
| chr17 | 62291452 | 62291582 | 0.000484821 | −24.71947368 | Hypo-methylated | NEPC\|CRPC-Adeno DMRs |
| chr17 | 62359799 | 62360154 | 0.000542154 | −23.16140351 | Hypo-methylated | NEPC\|CRPC-Adeno DMRs |
| chr17 | 63553578 | 63554707 | 0.000549237 | 31.0786132 | Hyper-methylated | NEPC\|CRPC-Adeno DMRs |
| chr17 | 64383923 | 64387249 | 0.001694183 | −20.34307992 | Hypo-methylated | NEPC\|CRPC-Adeno DMRs |
| chr17 | 64724456 | 64733778 | 0.003113626 | −26.88749816 | Hypo-methylated | NEPC\|CRPC-Adeno DMRs |
| chr17 | 65487278 | 65487463 | 5.17E−06 | −38.01818182 | Hypo-methylated | NEPC\|CRPC-Adeno DMRs |
| chr17 | 69077025 | 69077237 | 0.000176134 | −37.46184211 | Hypo-methylated | NEPC\|CRPC-Adeno DMRs |
| chr17 | 70416119 | 70416320 | 0.000549237 | −39.20300752 | Hypo-methylated | NEPC\|CRPC-Adeno DMRs |
| chr17 | 70417006 | 70436512 | 2.03E−05 | −27.51271442 | Hypo-methylated | NEPC\|CRPC-Adeno DMRs |
| chr17 | 70657568 | 70657830 | 5.17E−06 | −28.76519139 | Hypo-methylated | NEPC\|CRPC-Adeno DMRs |
| chr17 | 70785266 | 70785335 | 0.000101637 | −26.20409357 | Hypo-methylated | NEPC\|CRPC-Adeno DMRs |
| chr17 | 71269338 | 71269422 | 0.001694183 | −35.15844298 | Hypo-methylated | NEPC\|CRPC-Adeno DMRs |
| chr17 | 71709442 | 71716007 | 0.000194541 | −40.33795879 | Hypo-methylated | NEPC\|CRPC-Adeno DMRs |
| chr17 | 71743875 | 71746259 | 2.91E−06 | −49.20547723 | Hypo-methylated | NEPC\|CRPC-Adeno DMRs |
| chr17 | 71898021 | 71899573 | 3.56E−05 | −20.20286669 | Hypo-methylated | NEPC\|CRPC-Adeno DMRs |
| chr17 | 72264298 | 72264379 | 0.003113626 | −20.49561404 | Hypo-methylated | NEPC\|CRPC-Adeno DMRs |
| chr17 | 72306141 | 72306312 | 1.70E−05 | −38.42149958 | Hypo-methylated | NEPC\|CRPC-Adeno DMRs |
| chr17 | 72322190 | 72336829 | 2.26E−09 | −26.16578621 | Hypo-methylated | NEPC\|CRPC-Adeno DMRs |
| chr17 | 72350558 | 72350690 | 0.000953892 | 37.96869195 | Hyper-methylated | NEPC\|CRPC-Adeno DMRs |
| chr17 | 72691140 | 72700930 | 0.001274051 | −24.78561404 | Hypo-methylated | NEPC\|CRPC-Adeno DMRs |
| chr17 | 72945526 | 72946377 | 0.00324027 | −25.96264108 | Hypo-methylated | NEPC\|CRPC-Adeno DMRs |
| chr17 | 73073323 | 73073413 | 0.001694183 | −24.91140351 | Hypo-methylated | NEPC\|CRPC-Adeno DMRs |
| chr17 | 73585636 | 73586234 | 7.65E−05 | −22.58378022 | Hypo-methylated | NEPC\|CRPC-Adeno DMRs |
| chr17 | 73612317 | 73619220 | 4.63E−09 | −25.56901729 | Hypo-methylated | NEPC\|CRPC-Adeno DMRs |
| chr17 | 73824014 | 73824138 | 1.62E−05 | −22.74502402 | Hypo-methylated | NEPC\|CRPC-Adeno DMRs |
| chr17 | 73831455 | 73832137 | 0.004686715 | −24.79876161 | Hypo-methylated | NEPC\|CRPC-Adeno DMRs |
| chr17 | 73904522 | 73906744 | 0.002094266 | −30.17200292 | Hypo-methylated | NEPC\|CRPC-Adeno DMRs |
| chr17 | 73913826 | 73914050 | 7.40E−07 | −28.62135363 | Hypo-methylated | NEPC\|CRPC-Adeno DMRs |
| chr17 | 74133918 | 74133974 | 1.70E−05 | −29.44555556 | Hypo-methylated | NEPC\|CRPC-Adeno DMRs |
| chr17 | 74138282 | 74138397 | 0.001545937 | −23.20337206 | Hypo-methylated | NEPC\|CRPC-Adeno DMRs |
| chr17 | 74141386 | 74141672 | 1.55E−06 | −40.81096491 | Hypo-methylated | NEPC\|CRPC-Adeno DMRs |
| chr17 | 74696785 | 74696845 | 0.003525992 | −24.9122807 | Hypo-methylated | NEPC\|CRPC-Adeno DMRs |
| chr17 | 74965090 | 74965740 | 1.81E−11 | −28.23060429 | Hypo-methylated | NEPC\|CRPC-Adeno DMRs |
| chr17 | 75106184 | 75112522 | 0.001545937 | 37.01316254 | Hyper-methylated | NEPC\|CRPC-Adeno DMRs |
| chr17 | 75321510 | 75321614 | 0.002976353 | 21.02773601 | Hyper-methylated | NEPC\|CRPC-Adeno DMRs |
| chr17 | 75525474 | 75525552 | 0.007136365 | 24.95817222 | Hyper-methylated | NEPC\|CRPC-Adeno DMRs |
| chr17 | 75544150 | 75556717 | 0.001904835 | 20.2547121 | Hyper-methylated | NEPC\|CRPC-Adeno DMRs |
| chr17 | 75557102 | 75558856 | 0.000101637 | −21.87983575 | Hypo-methylated | NEPC\|CRPC-Adeno DMRs |
| chr17 | 75640945 | 75641329 | 0.001802623 | −22.8037594 | Hypo-methylated | NEPC\|CRPC-Adeno DMRs |
| chr17 | 75692468 | 75692572 | 0.000176134 | −20.03092105 | Hypo-methylated | NEPC\|CRPC-Adeno DMRs |
| chr17 | 76112799 | 76113775 | 2.96E−05 | −37.15700215 | Hypo-methylated | NEPC\|CRPC-Adeno DMRs |
| chr17 | 76264541 | 76265946 | 0.0001415 | −29.49485346 | Hypo-methylated | NEPC\|CRPC-Adeno DMRs |
| chr17 | 76471490 | 76471694 | 0.000263673 | −29.50263158 | Hypo-methylated | NEPC\|CRPC-Adeno DMRs |
| chr17 | 76673182 | 76673287 | 0.000549237 | −25.07744361 | Hypo-methylated | NEPC\|CRPC-Adeno DMRs |
| chr17 | 76899098 | 76899424 | 0.000549237 | −33.26137894 | Hypo-methylated | NEPC\|CRPC-Adeno DMRs |
| chr17 | 76926119 | 76926612 | 2.77E−06 | −32.79546668 | Hypo-methylated | NEPC\|CRPC-Adeno DMRs |
| chr17 | 76973622 | 76977745 | 3.30E−09 | −27.54742565 | Hypo-methylated | NEPC\|CRPC-Adeno DMRs |
| chr17 | 77024092 | 77028305 | 1.09E−05 | 29.91227472 | Hyper-methylated | NEPC\|CRPC-Adeno DMRs |
| chr17 | 77102634 | 77104721 | 3.94E−07 | −21.20069618 | Hypo-methylated | NEPC\|CRPC-Adeno DMRs |
| chr17 | 77644505 | 77654024 | 2.44E−05 | −31.51285282 | Hypo-methylated | NEPC\|CRPC-Adeno DMRs |
| chr17 | 77688543 | 77688776 | 0.000808696 | −21.95717593 | Hypo-methylated | NEPC\|CRPC-Adeno DMRs |
| chr17 | 77916790 | 77916997 | 4.03E−08 | 20.96640351 | Hyper-methylated | NEPC\|CRPC-Adeno DMRs |
| chr17 | 78076207 | 78079672 | 4.56E−08 | −41.22212476 | Hypo-methylated | NEPC\|CRPC-Adeno DMRs |
| chr17 | 78169398 | 78169650 | 0.000832692 | −33.81315789 | Hypo-methylated | NEPC\|CRPC-Adeno DMRs |
| chr17 | 78321224 | 78321333 | 0.009458442 | 21.23859649 | Hyper-methylated | NEPC\|CRPC-Adeno DMRs |
| chr17 | 78395361 | 78396367 | 0.000101637 | −40.49078947 | Hypo-methylated | NEPC\|CRPC-Adeno DMRs |
| chr17 | 78417869 | 78417933 | 1.03E−05 | −27.27763961 | Hypo-methylated | NEPC\|CRPC-Adeno DMRs |
| chr17 | 78438317 | 78447069 | 0.000117502 | −25.85335104 | Hypo-methylated | NEPC\|CRPC-Adeno DMRs |
| chr17 | 78598698 | 78620197 | 6.49E−05 | −40.54287967 | Hypo-methylated | NEPC\|CRPC-Adeno DMRs |
| chr17 | 78652902 | 78652975 | 0.003525992 | −41.20272904 | Hypo-methylated | NEPC\|CRPC-Adeno DMRs |
| chr17 | 78736046 | 78746738 | 2.76E−05 | −37.71010955 | Hypo-methylated | NEPC\|CRPC-Adeno DMRs |
| chr17 | 78759749 | 78764730 | 2.50E−06 | −30.03933723 | Hypo-methylated | NEPC\|CRPC-Adeno DMRs |
| chr17 | 78819994 | 78820445 | 0.000670739 | −33.34703302 | Hypo-methylated | NEPC\|CRPC-Adeno DMRs |
| chr17 | 78832469 | 78833739 | 0.003808143 | −23.88322522 | Hypo-methylated | NEPC\|CRPC-Adeno DMRs |
| chr17 | 78872433 | 78875062 | 4.95E−06 | −27.96011905 | Hypo-methylated | NEPC\|CRPC-Adeno DMRs |
| chr17 | 78882738 | 78888279 | 5.50E−05 | −34.47726046 | Hypo-methylated | NEPC\|CRPC-Adeno DMRs |
| chr17 | 78893612 | 78894478 | 9.61E−06 | −26.53827751 | Hypo-methylated | NEPC\|CRPC-Adeno DMRs |
| chr17 | 79007403 | 79007879 | 0.002094266 | 32.9994152 | Hyper-methylated | NEPC\|CRPC-Adeno DMRs |
| chr17 | 79071073 | 79071801 | 2.31E−05 | 21.55921594 | Hyper-methylated | NEPC\|CRPC-Adeno DMRs |
| chr17 | 79219751 | 79219890 | 0.001608723 | −27.23369023 | Hypo-methylated | NEPC\|CRPC-Adeno DMRs |
| chr17 | 79224043 | 79229561 | 1.80E−08 | −34.36076355 | Hypo-methylated | NEPC\|CRPC-Adeno DMRs |
| chr17 | 79276696 | 79277025 | 8.87E−06 | −20.83618421 | Hypo-methylated | NEPC\|CRPC-Adeno DMRs |
| chr17 | 79383521 | 79383993 | 3.05E−05 | −35.95336338 | Hypo-methylated | NEPC\|CRPC-Adeno DMRs |
| chr17 | 79388538 | 79390891 | 1.44E−13 | −32.99476877 | Hypo-methylated | NEPC\|CRPC-Adeno DMRs |
| chr17 | 79393653 | 79394177 | 0.009285355 | −31.57918801 | Hypo-methylated | NEPC\|CRPC-Adeno DMRs |
| chr17 | 79400097 | 79400753 | 0.001492528 | −29.51705059 | Hypo-methylated | NEPC\|CRPC-Adeno DMRs |
| chr17 | 79532081 | 79532452 | 4.24E−06 | 25.87819549 | Hyper-methylated | NEPC\|CRPC-Adeno DMRs |
| chr17 | 79799496 | 79799525 | 0.000184724 | 27.5678525 | Hyper-methylated | NEPC\|CRPC-Adeno DMRs |
| chr17 | 79970290 | 79971389 | 2.44E−05 | −21.41785714 | Hypo-methylated | NEPC\|CRPC-Adeno DMRs |
| chr17 | 80236905 | 80237030 | 0.000200814 | −32.95947368 | Hypo-methylated | NEPC\|CRPC-Adeno DMRs |
| chr17 | 80294643 | 80294804 | 0.005737741 | −22.66072125 | Hypo-methylated | NEPC\|CRPC-Adeno DMRs |
| chr17 | 80372750 | 80373728 | 0.001221669 | 54.03907686 | Hyper-methylated | NEPC\|CRPC-Adeno DMRs |
| chr17 | 80401424 | 80401915 | 1.11E−05 | 24.13307469 | Hyper-methylated | NEPC\|CRPC-Adeno DMRs |
| chr17 | 80454497 | 80454782 | 0.000903819 | −21.5033029 | Hypo-methylated | NEPC\|CRPC-Adeno DMRs |
| chr17 | 80517975 | 80518372 | 4.34E−06 | −23.41949746 | Hypo-methylated | NEPC\|CRPC-Adeno DMRs |
| chr17 | 80648763 | 80648968 | 0.002432731 | −28.26466165 | Hypo-methylated | NEPC\|CRPC-Adeno DMRs |
| chr17 | 80788028 | 80788377 | 1.41E−06 | −26.69938443 | Hypo-methylated | NEPC\|CRPC-Adeno DMRs |
| chr17 | 80914629 | 80915867 | 8.25E−08 | −41.06770335 | Hypo-methylated | NEPC\|CRPC-Adeno DMRs |
| chr17 | 80968121 | 80968398 | 2.35E−05 | −29.03596344 | Hypo-methylated | NEPC\|CRPC-Adeno DMRs |
| chr17 | 81014810 | 81015965 | 0.001802623 | −22.12938596 | Hypo-methylated | NEPC\|CRPC-Adeno DMRs |
| chr17 | 81125962 | 81126730 | 1.11E−05 | −27.3560307 | Hypo-methylated | NEPC\|CRPC-Adeno DMRs |
| chr18 | 75658 | 75998 | 4.82E−06 | 36.99894344 | Hyper-methylated | NEPC\|CRPC-Adeno DMRs |
| chr18 | 334794 | 449798 | 0.001660911 | −20.74100246 | Hypo-methylated | NEPC\|CRPC-Adeno DMRs |
| chr18 | 4147291 | 4337389 | 3.27E−07 | −34.36188494 | Hypo-methylated | NEPC\|CRPC-Adeno DMRs |
| chr18 | 5543159 | 5543932 | 1.16E−23 | 32.2432484 | Hyper-methylated | NEPC\|CRPC-Adeno DMRs |
| chr18 | 5545242 | 5577549 | 0.001065261 | −28.16790997 | Hypo-methylated | NEPC\|CRPC-Adeno DMRs |
| chr18 | 5628489 | 5630725 | 5.25E−46 | 31.23235399 | Hyper-methylated | NEPC\|CRPC-Adeno DMRs |
| chr18 | 5890293 | 5891826 | 8.90E−46 | 43.18632076 | Hyper-methylated | NEPC\|CRPC-Adeno DMRs |
| chr18 | 6556270 | 6634706 | 1.41E−07 | −36.4994152 | Hypo-methylated | NEPC\|CRPC-Adeno DMRs |
| chr18 | 6797808 | 6918975 | 3.57E−06 | −22.01492342 | Hypo-methylated | NEPC\|CRPC-Adeno DMRs |
| chr18 | 7068147 | 7115960 | 2.14E−07 | −24.11120635 | Hypo-methylated | NEPC\|CRPC-Adeno DMRs |
| chr18 | 7120609 | 7172357 | 6.66E−05 | −20.69170475 | Hypo-methylated | NEPC\|CRPC-Adeno DMRs |
| chr18 | 7370850 | 7371021 | 5.50E−05 | 24.72956465 | Hyper-methylated | NEPC\|CRPC-Adeno DMRs |
| chr18 | 7677985 | 7804451 | 0.000921835 | −21.2020202 | Hypo-methylated | NEPC\|CRPC-Adeno DMRs |
| chr18 | 8394353 | 8394533 | 0.001694183 | −48.30318627 | Hypo-methylated | NEPC\|CRPC-Adeno DMRs |
| chr18 | 8659584 | 8660112 | 8.97E−08 | −55.51884122 | Hypo-methylated | NEPC\|CRPC-Adeno DMRs |
| chr18 | 8844768 | 8845067 | 1.70E−05 | −43.19979016 | Hypo-methylated | NEPC\|CRPC-Adeno DMRs |
| chr18 | 8912451 | 8935685 | 0.001065261 | −37.41020143 | Hypo-methylated | NEPC\|CRPC-Adeno DMRs |
| chr18 | 11149243 | 11149551 | 2.78E−10 | 25.70827947 | Hyper-methylated | NEPC\|CRPC-Adeno DMRs |
| chr18 | 11697664 | 11751264 | 3.61E−07 | −30.46443807 | Hypo-methylated | NEPC\|CRPC-Adeno DMRs |
| chr18 | 11751678 | 11751841 | 0.000286322 | 21.92816416 | Hyper-methylated | NEPC\|CRPC-Adeno DMRs |
| chr18 | 11917723 | 11943273 | 6.65E−05 | −26.35537562 | Hypo-methylated | NEPC\|CRPC-Adeno DMRs |
| chr18 | 11994232 | 12006937 | 0.000950523 | −25.66920795 | Hypo-methylated | NEPC\|CRPC-Adeno DMRs |
| chr18 | 12139501 | 12236405 | 8.97E−07 | −22.55416741 | Hypo-methylated | NEPC\|CRPC-Adeno DMRs |
| chr18 | 12247455 | 12247499 | 0.001694183 | −34.97354108 | Hypo-methylated | NEPC\|CRPC-Adeno DMRs |
| chr18 | 13164513 | 13164650 | 0.001694183 | −27.58918129 | Hypo-methylated | NEPC\|CRPC-Adeno DMRs |
| chr18 | 13765981 | 13821981 | 5.92E−05 | −20.73623113 | Hypo-methylated | NEPC\|CRPC-Adeno DMRs |
| chr18 | 14145797 | 14173352 | 8.16E−09 | −28.74286327 | Hypo-methylated | NEPC\|CRPC-Adeno DMRs |
| chr18 | 14179084 | 14179364 | 0.000926684 | 41.99836466 | Hyper-methylated | NEPC\|CRPC-Adeno DMRs |
| chr18 | 14221851 | 14393937 | 2.23E−18 | −31.1325109 | Hypo-methylated | NEPC\|CRPC-Adeno DMRs |
| chr18 | 14431272 | 14431428 | 2.18E−08 | 27.72054411 | Hyper-methylated | NEPC\|CRPC-Adeno DMRs |
| chr18 | 15197752 | 15197801 | 0.001694183 | 33.80175439 | Hyper-methylated | NEPC\|CRPC-Adeno DMRs |
| chr18 | 15261217 | 15261271 | 0.000581135 | −31.94736842 | Hypo-methylated | NEPC\|CRPC-Adeno DMRs |
| chr18 | 15329900 | 18516173 | 0.000672574 | −27.39216616 | Hypo-methylated | NEPC\|CRPC-Adeno DMRs |
| chr18 | 19793485 | 19803796 | 0.007136365 | −25.2424812 | Hypo-methylated | NEPC\|CRPC-Adeno DMRs |
| chr18 | 21437853 | 21437989 | 0.001012154 | −24.88293651 | Hypo-methylated | NEPC\|CRPC-Adeno DMRs |
| chr18 | 22965138 | 23570664 | 3.47E−05 | −20.70556205 | Hypo-methylated | NEPC\|CRPC-Adeno DMRs |
| chr18 | 23869543 | 23986382 | 0.004750844 | −21.16466165 | Hypo-methylated | NEPC\|CRPC-Adeno DMRs |
| chr18 | 24043721 | 24128145 | 1.46E−07 | 38.88147122 | Hyper-methylated | NEPC\|CRPC-Adeno DMRs |
| chr18 | 24301879 | 24348460 | 5.50E−05 | −41.08638262 | Hypo-methylated | NEPC\|CRPC-Adeno DMRs |
| chr18 | 24598939 | 24681481 | 4.65E−07 | −43.09462358 | Hypo-methylated | NEPC\|CRPC-Adeno DMRs |
| chr18 | 25117072 | 25157386 | 0.000396595 | −33.17815187 | Hypo-methylated | NEPC\|CRPC-Adeno DMRs |
| chr18 | 25366258 | 25442245 | 1.17E−08 | −49.37024854 | Hypo-methylated | NEPC\|CRPC-Adeno DMRs |
| chr18 | 25545804 | 25662301 | 0.009274146 | −23.39980269 | Hypo-methylated | NEPC\|CRPC-Adeno DMRs |
| chr18 | 29719797 | 29723172 | 0.000908052 | −43.04432483 | Hypo-methylated | NEPC\|CRPC-Adeno DMRs |
| chr18 | 30896422 | 30896504 | 0.000549237 | −32.4375 | Hypo-methylated | NEPC\|CRPC-Adeno DMRs |
| chr18 | 31020556 | 31020916 | 7.29E−15 | 33.2286151 | Hyper-methylated | NEPC\|CRPC-Adeno DMRs |
| chr18 | 33768004 | 33768064 | 1.70E−05 | 28.46684211 | Hyper-methylated | NEPC\|CRPC-Adeno DMRs |
| chr18 | 34850803 | 34866666 | 3.07E−13 | 39.61752777 | Hyper-methylated | NEPC\|CRPC-Adeno DMRs |
| chr18 | 35014283 | 35059155 | 5.34E−05 | 24.68867345 | Hyper-methylated | NEPC\|CRPC-Adeno DMRs |
| chr18 | 42324317 | 42325096 | 2.55E−13 | −29.35696572 | Hypo-methylated | NEPC\|CRPC-Adeno DMRs |
| chr18 | 42901586 | 42901643 | 0.001694183 | −39.11783626 | Hypo-methylated | NEPC\|CRPC-Adeno DMRs |
| chr18 | 43732943 | 43732998 | 0.001694183 | −34.38283208 | Hypo-methylated | NEPC\|CRPC-Adeno DMRs |
| chr18 | 44773018 | 44774815 | 3.88E−27 | 35.39995946 | Hyper-methylated | NEPC\|CRPC-Adeno DMRs |
| chr18 | 44780729 | 44790503 | 1.02E−19 | 27.31399674 | Hyper-methylated | NEPC\|CRPC-Adeno DMRs |
| chr18 | 46386305 | 46386822 | 5.01E−05 | −20.09974937 | Hypo-methylated | NEPC\|CRPC-Adeno DMRs |
| chr18 | 47026017 | 47026653 | 0.007136365 | −21.66766917 | Hypo-methylated | NEPC\|CRPC-Adeno DMRs |
| chr18 | 47812965 | 47813193 | 4.65E−07 | −24.69747526 | Hypo-methylated | NEPC\|CRPC-Adeno DMRs |
| chr18 | 48637066 | 48637129 | 0.000549237 | 26.20841688 | Hyper-methylated | NEPC\|CRPC-Adeno DMRs |
| chr18 | 51921754 | 52019720 | 0.004750844 | −35.99744152 | Hypo-methylated | NEPC\|CRPC-Adeno DMRs |
| chr18 | 52508234 | 52591105 | 4.65E−07 | −26.07759784 | Hypo-methylated | NEPC\|CRPC-Adeno DMRs |
| chr18 | 53047047 | 53238004 | 0.000549237 | −33.22742272 | Hypo-methylated | NEPC\|CRPC-Adeno DMRs |
| chr18 | 53269037 | 53435792 | 1.71E−10 | −36.25301297 | Hypo-methylated | NEPC\|CRPC-Adeno DMRs |
| chr18 | 53447476 | 53447803 | 3.10E−14 | 33.32333865 | Hyper-methylated | NEPC\|CRPC-Adeno DMRs |
| chr18 | 55469022 | 55469165 | 4.95E−05 | −20.90794634 | Hypo-methylated | NEPC\|CRPC-Adeno DMRs |
| chr18 | 56259727 | 56260226 | 0.001065261 | −28.10994152 | Hypo-methylated | NEPC\|CRPC-Adeno DMRs |
| chr18 | 56934972 | 56935515 | 0.000218899 | 27.72535088 | Hyper-methylated | NEPC\|CRPC-Adeno DMRs |
| chr18 | 56940842 | 56941558 | 1.31E−09 | 28.36486693 | Hyper-methylated | NEPC\|CRPC-Adeno DMRs |
| chr18 | 57106203 | 57357805 | 3.23E−06 | −34.68633501 | Hypo-methylated | NEPC\|CRPC-Adeno DMRs |
| chr18 | 57363762 | 57364650 | 1.76E−25 | 40.22391517 | Hyper-methylated | NEPC\|CRPC-Adeno DMRs |
| chr18 | 57366036 | 57556747 | 1.01E−07 | −30.65155964 | Hypo-methylated | NEPC\|CRPC-Adeno DMRs |
| chr18 | 57640688 | 57677912 | 0.000152004 | −24.32308222 | Hypo-methylated | NEPC\|CRPC-Adeno DMRs |
| chr18 | 59001049 | 59001824 | 3.09E−08 | 20.67838477 | Hyper-methylated | NEPC\|CRPC-Adeno DMRs |
| chr18 | 60263503 | 60263750 | 2.01E−08 | 30.16628242 | Hyper-methylated | NEPC\|CRPC-Adeno DMRs |
| chr18 | 67069032 | 67069553 | 7.87E−11 | 27.32008772 | Hyper-methylated | NEPC\|CRPC-Adeno DMRs |
| chr18 | 67136733 | 67136880 | 0.000324528 | −20.78318713 | Hypo-methylated | NEPC\|CRPC-Adeno DMRs |
| chr18 | 67972299 | 67972368 | 0.009458442 | 21.12631579 | Hyper-methylated | NEPC\|CRPC-Adeno DMRs |
| chr18 | 68166726 | 68166932 | 7.41E−07 | −55.29662309 | Hypo-methylated | NEPC\|CRPC-Adeno DMRs |
| chr18 | 70536311 | 70536519 | 7.87E−11 | 34.41356381 | Hyper-methylated | NEPC\|CRPC-Adeno DMRs |
| chr18 | 71610877 | 71611303 | 0.003113626 | −24.34640523 | Hypo-methylated | NEPC\|CRPC-Adeno DMRs |
| chr18 | 72153858 | 72153991 | 1.55E−06 | −41.42650785 | Hypo-methylated | NEPC\|CRPC-Adeno DMRs |
| chr18 | 72960958 | 72961205 | 1.70E−05 | −34.94250774 | Hypo-methylated | NEPC\|CRPC-Adeno DMRs |
| chr18 | 73167240 | 73168223 | 2.74E−14 | 36.76761988 | Hyper-methylated | NEPC\|CRPC-Adeno DMRs |
| chr18 | 73628239 | 73628323 | 8.97E−08 | 32.73611807 | Hyper-methylated | NEPC\|CRPC-Adeno DMRs |
| chr18 | 74103815 | 74107798 | 0.001883547 | −25.66330666 | Hypo-methylated | NEPC\|CRPC-Adeno DMRs |
| chr18 | 74754672 | 74754750 | 0.001694183 | −44.22368421 | Hypo-methylated | NEPC\|CRPC-Adeno DMRs |
| chr18 | 74805178 | 74805233 | 0.000176134 | −57.8546627 | Hypo-methylated | NEPC\|CRPC-Adeno DMRs |
| chr18 | 74961918 | 74962221 | 2.65E−10 | 31.43779694 | Hyper-methylated | NEPC\|CRPC-Adeno DMRs |
| chr18 | 74962315 | 74964223 | 1.69E−15 | 33.30443051 | Hyper-methylated | NEPC\|CRPC-Adeno DMRs |
| chr18 | 76069561 | 76069618 | 3.18E−06 | 27.39018843 | Hyper-methylated | NEPC\|CRPC-Adeno DMRs |
| chr18 | 76694294 | 76695037 | 0.001694183 | −36.48888889 | Hypo-methylated | NEPC\|CRPC-Adeno DMRs |
| chr18 | 77147768 | 77147847 | 0.000711935 | −31.27134503 | Hypo-methylated | NEPC\|CRPC-Adeno DMRs |
| chr18 | 77244836 | 77245030 | 1.68E−07 | −21.14505582 | Hypo-methylated | NEPC\|CRPC-Adeno DMRs |
| chr18 | 77290965 | 77291471 | 3.80E−05 | −25.71487921 | Hypo-methylated | NEPC\|CRPC-Adeno DMRs |
| chr18 | 77313296 | 77316500 | 1.75E−06 | −29.56169959 | Hypo-methylated | NEPC\|CRPC-Adeno DMRs |
| chr18 | 77320467 | 77325890 | 1.65E−08 | −43.11216158 | Hypo-methylated | NEPC\|CRPC-Adeno DMRs |
| chr18 | 77372704 | 77373764 | 2.37E−06 | −24.37929997 | Hypo-methylated | NEPC\|CRPC-Adeno DMRs |
| chr18 | 77512369 | 77512452 | 0.001694183 | 23.98040936 | Hyper-methylated | NEPC\|CRPC-Adeno DMRs |
| chr18 | 77534899 | 77538503 | 0.000601653 | −25.78905597 | Hypo-methylated | NEPC\|CRPC-Adeno DMRs |
| chr18 | 77547894 | 77548089 | 4.65E−07 | 26.78505247 | Hyper-methylated | NEPC\|CRPC-Adeno DMRs |
| chr18 | 77557063 | 77557318 | 9.09E−05 | −29.81704261 | Hypo-methylated | NEPC\|CRPC-Adeno DMRs |
| chr18 | 77607722 | 77608332 | 0.005737741 | −44.14912281 | Hypo-methylated | NEPC\|CRPC-Adeno DMRs |
| chr18 | 77627555 | 77632025 | 1.15E−05 | −23.34253057 | Hypo-methylated | NEPC\|CRPC-Adeno DMRs |
| chr19 | 397436 | 397602 | 0.000549237 | −31.90412305 | Hypo-methylated | NEPC\|CRPC-Adeno DMRs |
| chr19 | 426998 | 427102 | 1.70E−05 | −27.41677527 | Hypo-methylated | NEPC\|CRPC-Adeno DMRs |
| chr19 | 489778 | 493649 | 9.70E−06 | 20.65651084 | Hyper-methylated | NEPC\|CRPC-Adeno DMRs |
| chr19 | 524073 | 525378 | 3.90E−06 | −23.64102792 | Hypo-methylated | NEPC\|CRPC-Adeno DMRs |
| chr19 | 591312 | 592180 | 6.98E−11 | 44.34577647 | Hyper-methylated | NEPC\|CRPC-Adeno DMRs |
| chr19 | 609127 | 609421 | 0.001455962 | −23.45263158 | Hypo-methylated | NEPC\|CRPC-Adeno DMRs |
| chr19 | 629784 | 630015 | 0.000291618 | −29.93349282 | Hypo-methylated | NEPC\|CRPC-Adeno DMRs |
| chr19 | 658494 | 662041 | 6.12E−06 | 23.05631866 | Hyper-methylated | NEPC\|CRPC-Adeno DMRs |
| chr19 | 740537 | 740726 | 5.50E−05 | −31.11140167 | Hypo-methylated | NEPC\|CRPC-Adeno DMRs |
| chr19 | 855570 | 855794 | 4.47E−05 | −23.57004548 | Hypo-methylated | NEPC\|CRPC-Adeno DMRs |
| chr19 | 870959 | 871390 | 4.55E−09 | −34.75051877 | Hypo-methylated | NEPC\|CRPC-Adeno DMRs |
| chr19 | 883736 | 884368 | 9.23E−10 | −44.98650953 | Hypo-methylated | NEPC\|CRPC-Adeno DMRs |
| chr19 | 898790 | 898951 | 0.005737741 | −22.13253589 | Hypo-methylated | NEPC\|CRPC-Adeno DMRs |
| chr19 | 900754 | 901044 | 0.000102266 | 22.0454363 | Hyper-methylated | NEPC\|CRPC-Adeno DMRs |
| chr19 | 903325 | 903540 | 4.01E−05 | −32.55983276 | Hypo-methylated | NEPC\|CRPC-Adeno DMRs |
| chr19 | 951603 | 954079 | 0.007271222 | 25.6958751 | Hyper-methylated | NEPC\|CRPC-Adeno DMRs |
| chr19 | 1041762 | 1042088 | 5.92E−05 | −23.26772982 | Hypo-methylated | NEPC\|CRPC-Adeno DMRs |
| chr19 | 1064147 | 1064410 | 4.24E−06 | −28.16687552 | Hypo-methylated | NEPC\|CRPC-Adeno DMRs |
| chr19 | 1106628 | 1108021 | 0.002023529 | 20.64123735 | Hyper-methylated | NEPC\|CRPC-Adeno DMRs |
| chr19 | 1241167 | 1241327 | 0.009458442 | −24.53534572 | Hypo-methylated | NEPC\|CRPC-Adeno DMRs |
| chr19 | 1292106 | 1292610 | 0.000381376 | −21.70368282 | Hypo-methylated | NEPC\|CRPC-Adeno DMRs |
| chr19 | 1340494 | 1342761 | 5.17E−06 | −31.29391555 | Hypo-methylated | NEPC\|CRPC-Adeno DMRs |
| chr19 | 1371048 | 1371087 | 5.50E−05 | −45.9707672 | Hypo-methylated | NEPC\|CRPC-Adeno DMRs |
| chr19 | 1397431 | 1397739 | 0.000341437 | −20.44259614 | Hypo-methylated | NEPC\|CRPC-Adeno DMRs |
| chr19 | 1505824 | 1506160 | 0.003503755 | −22.34851565 | Hypo-methylated | NEPC\|CRPC-Adeno DMRs |
| chr19 | 1508528 | 1511932 | 5.57E−07 | −24.11860288 | Hypo-methylated | NEPC\|CRPC-Adeno DMRs |
| chr19 | 1520397 | 1520541 | 0.001694183 | −48.78637771 | Hypo-methylated | NEPC\|CRPC-Adeno DMRs |
| chr19 | 1572254 | 1574585 | 0.000700082 | −24.26105795 | Hypo-methylated | NEPC\|CRPC-Adeno DMRs |
| chr19 | 1578712 | 1578832 | 0.001694183 | −30.42807018 | Hypo-methylated | NEPC\|CRPC-Adeno DMRs |
| chr19 | 1691231 | 1698714 | 0.002602569 | −21.43010379 | Hypo-methylated | NEPC\|CRPC-Adeno DMRs |
| chr19 | 1719324 | 1719371 | 0.007136365 | −24.10827068 | Hypo-methylated | NEPC\|CRPC-Adeno DMRs |
| chr19 | 1729494 | 1734544 | 1.09E−05 | −22.84239881 | Hypo-methylated | NEPC\|CRPC-Adeno DMRs |
| chr19 | 1736641 | 1739365 | 0.000377187 | −22.94623206 | Hypo-methylated | NEPC\|CRPC-Adeno DMRs |
| chr19 | 1775452 | 1775525 | 0.008187764 | 20.59449448 | Hyper-methylated | NEPC\|CRPC-Adeno DMRs |
| chr19 | 1777638 | 1787193 | 6.79E−05 | −24.23056121 | Hypo-methylated | NEPC\|CRPC-Adeno DMRs |
| chr19 | 1826552 | 1826882 | 0.001694183 | 27.90158024 | Hyper-methylated | NEPC\|CRPC-Adeno DMRs |
| chr19 | 2016311 | 2016515 | 0.000703636 | −24.27866411 | Hypo-methylated | NEPC\|CRPC-Adeno DMRs |
| chr19 | 2170644 | 2171709 | 0.000291618 | −45.5170938 | Hypo-methylated | NEPC\|CRPC-Adeno DMRs |
| chr19 | 2213253 | 2213405 | 0.007136365 | −20.2569856 | Hypo-methylated | NEPC\|CRPC-Adeno DMRs |
| chr19 | 2255463 | 2257226 | 0.00039263 | −30.38435673 | Hypo-methylated | NEPC\|CRPC-Adeno DMRs |
| chr19 | 2268262 | 2268398 | 0.001802623 | 24.13082707 | Hyper-methylated | NEPC\|CRPC-Adeno DMRs |
| chr19 | 2299060 | 2299392 | 0.000549237 | −54.95621196 | Hypo-methylated | NEPC\|CRPC-Adeno DMRs |
| chr19 | 2324328 | 2324440 | 0.001549499 | −21.99770259 | Hypo-methylated | NEPC\|CRPC-Adeno DMRs |
| chr19 | 2513031 | 2513303 | 6.45E−06 | −25.6202879 | Hypo-methylated | NEPC\|CRPC-Adeno DMRs |
| chr19 | 2518509 | 2518637 | 0.00158762 | −29.37854251 | Hypo-methylated | NEPC\|CRPC-Adeno DMRs |
| chr19 | 2525152 | 2525272 | 9.61E−06 | −20.60388756 | Hypo-methylated | NEPC\|CRPC-Adeno DMRs |
| chr19 | 2634461 | 2635112 | 3.22E−05 | −29.63523392 | Hypo-methylated | NEPC\|CRPC-Adeno DMRs |
| chr19 | 2645923 | 2664817 | 8.45E−07 | −23.25229757 | Hypo-methylated | NEPC\|CRPC-Adeno DMRs |
| chr19 | 2867352 | 2867492 | 1.37E−07 | −28.72671261 | Hypo-methylated | NEPC\|CRPC-Adeno DMRs |
| chr19 | 2889171 | 2892153 | 0.000889013 | −20.6314198 | Hypo-methylated | NEPC\|CRPC-Adeno DMRs |
| chr19 | 3046352 | 3046594 | 0.001274051 | −27.18400376 | Hypo-methylated | NEPC\|CRPC-Adeno DMRs |
| chr19 | 3053360 | 3053625 | 0.000263673 | −25.58976035 | Hypo-methylated | NEPC\|CRPC-Adeno DMRs |
| chr19 | 3195923 | 3196751 | 1.69E−08 | −27.85969346 | Hypo-methylated | NEPC\|CRPC-Adeno DMRs |
| chr19 | 3197038 | 3197177 | 0.002376793 | −26.63206445 | Hypo-methylated | NEPC\|CRPC-Adeno DMRs |
| chr19 | 3220170 | 3221345 | 0.000908052 | −31.26391452 | Hypo-methylated | NEPC\|CRPC-Adeno DMRs |
| chr19 | 3290504 | 3293654 | 0.001904835 | −21.6261412 | Hypo-methylated | NEPC\|CRPC-Adeno DMRs |
| chr19 | 3367729 | 3374005 | 9.06E−10 | 20.2080845 | Hyper-methylated | NEPC\|CRPC-Adeno DMRs |
| chr19 | 3398658 | 3398810 | 0.000467288 | 30.07544839 | Hyper-methylated | NEPC\|CRPC-Adeno DMRs |
| chr19 | 3571951 | 3572019 | 0.00222033 | −26.78684211 | Hypo-methylated | NEPC\|CRPC-Adeno DMRs |
| chr19 | 3644460 | 3645475 | 0.003652661 | 20.18509503 | Hyper-methylated | NEPC\|CRPC-Adeno DMRs |
| chr19 | 3775063 | 3783899 | 7.65E−06 | −21.10231755 | Hypo-methylated | NEPC\|CRPC-Adeno DMRs |
| chr19 | 3784322 | 3784436 | 0.000549237 | −23.7481203 | Hypo-methylated | NEPC\|CRPC-Adeno DMRs |
| chr19 | 3820198 | 3821432 | 2.04E−10 | −27.78390977 | Hypo-methylated | NEPC\|CRPC-Adeno DMRs |
| chr19 | 3823603 | 3825768 | 4.74E−07 | −25.81743421 | Hypo-methylated | NEPC\|CRPC-Adeno DMRs |
| chr19 | 3855628 | 3860997 | 0.000711935 | −22.40219298 | Hypo-methylated | NEPC\|CRPC-Adeno DMRs |
| chr19 | 3937200 | 3938364 | 1.74E−05 | −20.9674843 | Hypo-methylated | NEPC\|CRPC-Adeno DMRs |
| chr19 | 4038720 | 4038745 | 0.000549237 | −24.40037594 | Hypo-methylated | NEPC\|CRPC-Adeno DMRs |
| chr19 | 4199744 | 4204316 | 0.00064342 | −20.48685013 | Hypo-methylated | NEPC\|CRPC-Adeno DMRs |
| chr19 | 4233060 | 4237715 | 0.000549237 | −24.74043044 | Hypo-methylated | NEPC\|CRPC-Adeno DMRs |
| chr19 | 4454848 | 4455414 | 0.009458442 | 22.6226817 | Hyper-methylated | NEPC\|CRPC-Adeno DMRs |
| chr19 | 4534974 | 4535447 | 5.49E−12 | −26.78065986 | Hypo-methylated | NEPC\|CRPC-Adeno DMRs |
| chr19 | 4538425 | 4538648 | 1.70E−05 | −39.23458855 | Hypo-methylated | NEPC\|CRPC-Adeno DMRs |
| chr19 | 4566605 | 4566669 | 1.55E−06 | −60.58903509 | Hypo-methylated | NEPC\|CRPC-Adeno DMRs |
| chr19 | 4571662 | 4576117 | 0.004786604 | −20.51624083 | Hypo-methylated | NEPC\|CRPC-Adeno DMRs |
| chr19 | 4846604 | 4852146 | 8.71E−05 | −28.89836814 | Hypo-methylated | NEPC\|CRPC-Adeno DMRs |
| chr19 | 4977546 | 4984579 | 3.38E−08 | −31.1306942 | Hypo-methylated | NEPC\|CRPC-Adeno DMRs |
| chr19 | 5035477 | 5038531 | 1.71E−10 | −31.90807804 | Hypo-methylated | NEPC\|CRPC-Adeno DMRs |
| chr19 | 5141239 | 5141793 | 5.10E−08 | 20.99015992 | Hyper-methylated | NEPC\|CRPC-Adeno DMRs |
| chr19 | 5307844 | 5307970 | 0.001694183 | 23.51666667 | Hyper-methylated | NEPC\|CRPC-Adeno DMRs |
| chr19 | 5517955 | 5523949 | 0.001274051 | −22.38698658 | Hypo-methylated | NEPC\|CRPC-Adeno DMRs |
| chr19 | 5727063 | 5760498 | 2.64E−05 | −20.03420297 | Hypo-methylated | NEPC\|CRPC-Adeno DMRs |
| chr19 | 5781208 | 5782328 | 0.000773732 | −30.07746289 | Hypo-methylated | NEPC\|CRPC-Adeno DMRs |
| chr19 | 5829066 | 5829466 | 9.12E−11 | −22.73137879 | Hypo-methylated | NEPC\|CRPC-Adeno DMRs |
| chr19 | 5956737 | 5964176 | 0.008985252 | −24.66587977 | Hypo-methylated | NEPC\|CRPC-Adeno DMRs |
| chr19 | 6249480 | 6259748 | 0.00064342 | 22.47845201 | Hyper-methylated | NEPC\|CRPC-Adeno DMRs |
| chr19 | 6284188 | 6311271 | 4.38E−06 | −20.64282176 | Hypo-methylated | NEPC\|CRPC-Adeno DMRs |
| chr19 | 6501345 | 6501464 | 0.000184724 | −36.68031189 | Hypo-methylated | NEPC\|CRPC-Adeno DMRs |
| chr19 | 6591008 | 6591117 | 0.005574264 | 20.09617225 | Hyper-methylated | NEPC\|CRPC-Adeno DMRs |
| chr19 | 6592081 | 6631207 | 0.000386347 | −21.22205115 | Hypo-methylated | NEPC\|CRPC-Adeno DMRs |
| chr19 | 7754225 | 7755302 | 1.14E−05 | −20.37647232 | Hypo-methylated | NEPC\|CRPC-Adeno DMRs |
| chr19 | 7854154 | 7855656 | 0.004735829 | −22.81671057 | Hypo-methylated | NEPC\|CRPC-Adeno DMRs |
| chr19 | 7934532 | 7937819 | 1.50E−12 | 20.10092043 | Hyper-methylated | NEPC\|CRPC-Adeno DMRs |
| chr19 | 7982226 | 7982768 | 3.13E−10 | −31.71591758 | Hypo-methylated | NEPC\|CRPC-Adeno DMRs |
| chr19 | 7991360 | 7992046 | 4.34E−06 | −41.16698233 | Hypo-methylated | NEPC\|CRPC-Adeno DMRs |
| chr19 | 8174155 | 8186255 | 2.54E−06 | −24.94782158 | Hypo-methylated | NEPC\|CRPC-Adeno DMRs |
| chr19 | 8273471 | 8273717 | 0.005899637 | 20.95734778 | Hyper-methylated | NEPC\|CRPC-Adeno DMRs |
| chr19 | 8595471 | 8625294 | 8.16E−08 | −26.5658489 | Hypo-methylated | NEPC\|CRPC-Adeno DMRs |
| chr19 | 9164807 | 9186967 | 1.82E−07 | −27.62189995 | Hypo-methylated | NEPC\|CRPC-Adeno DMRs |
| chr19 | 9473272 | 9473869 | 1.56E−05 | 26.21847536 | Hyper-methylated | NEPC\|CRPC-Adeno DMRs |
| chr19 | 9819060 | 9847697 | 0.000114041 | −25.02371804 | Hypo-methylated | NEPC\|CRPC-Adeno DMRs |
| chr19 | 9992040 | 9996397 | 2.14E−05 | −51.33445419 | Hypo-methylated | NEPC\|CRPC-Adeno DMRs |
| chr19 | 10124042 | 10124310 | 9.81E−05 | −22.89555199 | Hypo-methylated | NEPC\|CRPC-Adeno DMRs |
| chr19 | 10142060 | 10194578 | 1.45E−07 | −24.21652772 | Hypo-methylated | NEPC\|CRPC-Adeno DMRs |
| chr19 | 10475546 | 10475642 | 0.000168572 | 25.91861598 | Hyper-methylated | NEPC\|CRPC-Adeno DMRs |
| chr19 | 10747083 | 10747203 | 0.002976353 | 41.37141278 | Hyper-methylated | NEPC\|CRPC-Adeno DMRs |
| chr19 | 11002954 | 11003232 | 3.45E−06 | −32.59229184 | Hypo-methylated | NEPC\|CRPC-Adeno DMRs |
| chr19 | 11034997 | 11036416 | 0.008985252 | 21.92821637 | Hyper-methylated | NEPC\|CRPC-Adeno DMRs |
| chr19 | 11086500 | 11090861 | 0.009458442 | 23.34473684 | Hyper-methylated | NEPC\|CRPC-Adeno DMRs |
| chr19 | 11113739 | 11113942 | 0.000549237 | 21.48270677 | Hyper-methylated | NEPC\|CRPC-Adeno DMRs |
| chr19 | 12758753 | 12759531 | 1.27E−07 | −30.11934089 | Hypo-methylated | NEPC\|CRPC-Adeno DMRs |
| chr19 | 13293066 | 13293140 | 0.001694183 | 37.1877193 | Hyper-methylated | NEPC\|CRPC-Adeno DMRs |
| chr19 | 13928585 | 13929121 | 1.50E−06 | −26.49755762 | Hypo-methylated | NEPC\|CRPC-Adeno DMRs |
| chr19 | 14043706 | 14044198 | 1.25E−15 | −41.84347648 | Hypo-methylated | NEPC\|CRPC-Adeno DMRs |
| chr19 | 14094260 | 14095280 | 0.000711935 | −36.1302794 | Hypo-methylated | NEPC\|CRPC-Adeno DMRs |
| chr19 | 14321876 | 14323164 | 0.001545937 | −23.16221108 | Hypo-methylated | NEPC\|CRPC-Adeno DMRs |
| chr19 | 14376207 | 14391911 | 4.77E−06 | −23.18042441 | Hypo-methylated | NEPC\|CRPC-Adeno DMRs |
| chr19 | 14542909 | 14543000 | 0.000200814 | 21.42210526 | Hyper-methylated | NEPC\|CRPC-Adeno DMRs |
| chr19 | 15066284 | 15083780 | 0.000549237 | −34.93791771 | Hypo-methylated | NEPC\|CRPC-Adeno DMRs |
| chr19 | 15219923 | 15221487 | 3.99E−18 | −26.88039503 | Hypo-methylated | NEPC\|CRPC-Adeno DMRs |
| chr19 | 15271918 | 15272337 | 3.84E−06 | −28.78340643 | Hypo-methylated | NEPC\|CRPC-Adeno DMRs |
| chr19 | 15310531 | 15310570 | 0.000176134 | 23.06138393 | Hyper-methylated | NEPC\|CRPC-Adeno DMRs |
| chr19 | 15926482 | 15939342 | 0.000711935 | −30.32028219 | Hypo-methylated | NEPC\|CRPC-Adeno DMRs |
| chr19 | 15956576 | 16010462 | 1.00E−07 | −26.48761912 | Hypo-methylated | NEPC\|CRPC-Adeno DMRs |
| chr19 | 16090684 | 16178030 | 1.29E−12 | −26.1360614 | Hypo-methylated | NEPC\|CRPC-Adeno DMRs |
| chr19 | 16221519 | 16221643 | 0.00222033 | 25.01343985 | Hyper-methylated | NEPC\|CRPC-Adeno DMRs |
| chr19 | 17012756 | 17068587 | 9.30E−08 | −23.43599941 | Hypo-methylated | NEPC\|CRPC-Adeno DMRs |
| chr19 | 17152290 | 17152703 | 0.007136365 | −29.3191889 | Hypo-methylated | NEPC\|CRPC-Adeno DMRs |
| chr19 | 17224465 | 17225643 | 0.008600948 | 25.63125975 | Hyper-methylated | NEPC\|CRPC-Adeno DMRs |
| chr19 | 17320350 | 17320561 | 0.000324528 | 26.46392974 | Hyper-methylated | NEPC\|CRPC-Adeno DMRs |
| chr19 | 17346089 | 17347033 | 1.63E−29 | 22.3191432 | Hyper-methylated | NEPC\|CRPC-Adeno DMRs |
| chr19 | 17397263 | 17397679 | 0.000176134 | −26.43925439 | Hypo-methylated | NEPC\|CRPC-Adeno DMRs |
| chr19 | 17438101 | 17438549 | 2.00E−06 | −26.23284302 | Hypo-methylated | NEPC\|CRPC-Adeno DMRs |
| chr19 | 17476882 | 17484998 | 0.000549237 | −29.94181287 | Hypo-methylated | NEPC\|CRPC-Adeno DMRs |
| chr19 | 17767082 | 17791361 | 2.70E−09 | 42.00301353 | Hyper-methylated | NEPC\|CRPC-Adeno DMRs |
| chr19 | 18065301 | 18076654 | 0.000934755 | −24.50326018 | Hypo-methylated | NEPC\|CRPC-Adeno DMRs |
| chr19 | 18211820 | 18213958 | 0.001802623 | −21.7943609 | Hypo-methylated | NEPC\|CRPC-Adeno DMRs |
| chr19 | 18267723 | 18268799 | 2.52E−06 | −26.3456126 | Hypo-methylated | NEPC\|CRPC-Adeno DMRs |
| chr19 | 18468371 | 18479875 | 0.000926684 | −26.7100731 | Hypo-methylated | NEPC\|CRPC-Adeno DMRs |
| chr19 | 18549549 | 18549735 | 0.000184724 | 24.71721897 | Hyper-methylated | NEPC\|CRPC-Adeno DMRs |
| chr19 | 18706030 | 18706289 | 0.000247501 | 24.763055 | Hyper-methylated | NEPC\|CRPC-Adeno DMRs |
| chr19 | 18836691 | 18837107 | 0.000549237 | −69.08478488 | Hypo-methylated | NEPC\|CRPC-Adeno DMRs |
| chr19 | 18867715 | 18868406 | 0.006890179 | −27.9128655 | Hypo-methylated | NEPC\|CRPC-Adeno DMRs |
| chr19 | 18872000 | 18872158 | 0.007136365 | 21.33221596 | Hyper-methylated | NEPC\|CRPC-Adeno DMRs |
| chr19 | 19002328 | 19005336 | 0.000313698 | −25.2620313 | Hypo-methylated | NEPC\|CRPC-Adeno DMRs |
| chr19 | 19215770 | 19216380 | 5.17E−06 | −37.44047284 | Hypo-methylated | NEPC\|CRPC-Adeno DMRs |
| chr19 | 19252221 | 19252284 | 0.002094266 | −30.58414555 | Hypo-methylated | NEPC\|CRPC-Adeno DMRs |
| chr19 | 19349980 | 19360041 | 0.005813728 | −21.91089486 | Hypo-methylated | NEPC\|CRPC-Adeno DMRs |
| chr19 | 22611431 | 22611590 | 0.00643661 | 25.75356037 | Hyper-methylated | NEPC\|CRPC-Adeno DMRs |
| chr19 | 22784339 | 22799262 | 0.000542154 | −21.31765019 | Hypo-methylated | NEPC\|CRPC-Adeno DMRs |
| chr19 | 22805745 | 22805846 | 0.000184724 | 31.28219623 | Hyper-methylated | NEPC\|CRPC-Adeno DMRs |
| chr19 | 22966950 | 22967166 | 5.75E−05 | 21.36034056 | Hyper-methylated | NEPC\|CRPC-Adeno DMRs |
| chr19 | 22978007 | 22989337 | 5.75E−05 | −28.93334795 | Hypo-methylated | NEPC\|CRPC-Adeno DMRs |
| chr19 | 22990180 | 22990407 | 1.51E−06 | 26.07800701 | Hyper-methylated | NEPC\|CRPC-Adeno DMRs |
| chr19 | 22990489 | 22990603 | 0.000542154 | 20.06485912 | Hyper-methylated | NEPC\|CRPC-Adeno DMRs |
| chr19 | 23457611 | 23457661 | 0.001694183 | −29.57105263 | Hypo-methylated | NEPC\|CRPC-Adeno DMRs |
| chr19 | 29218495 | 29218628 | 0.000118331 | 20.92792237 | Hyper-methylated | NEPC\|CRPC-Adeno DMRs |
| chr19 | 29284406 | 29284930 | 2.72E−15 | 24.10907843 | Hyper-methylated | NEPC\|CRPC-Adeno DMRs |
| chr19 | 30109437 | 30135653 | 0.007136365 | −25.17133807 | Hypo-methylated | NEPC\|CRPC-Adeno DMRs |
| chr19 | 30157793 | 30158044 | 0.006890179 | 20.63926155 | Hyper-methylated | NEPC\|CRPC-Adeno DMRs |
| chr19 | 30339563 | 30347196 | 0.000124569 | −22.10334394 | Hypo-methylated | NEPC\|CRPC-Adeno DMRs |
| chr19 | 30647757 | 30701295 | 0.000246327 | −24.59190846 | Hypo-methylated | NEPC\|CRPC-Adeno DMRs |
| chr19 | 31111162 | 31142115 | 3.29E−08 | −26.99659526 | Hypo-methylated | NEPC\|CRPC-Adeno DMRs |
| chr19 | 31217434 | 31226491 | 0.000392904 | −21.23995396 | Hypo-methylated | NEPC\|CRPC-Adeno DMRs |
| chr19 | 31231444 | 31237122 | 0.000716662 | −22.9479023 | Hypo-methylated | NEPC\|CRPC-Adeno DMRs |
| chr19 | 31411329 | 31450495 | 4.74E−05 | −25.87915622 | Hypo-methylated | NEPC\|CRPC-Adeno DMRs |
| chr19 | 31601388 | 31619444 | 0.001068403 | −30.9933845 | Hypo-methylated | NEPC\|CRPC-Adeno DMRs |
| chr19 | 31637152 | 31640642 | 2.14E−05 | −34.25915524 | Hypo-methylated | NEPC\|CRPC-Adeno DMRs |
| chr19 | 31756020 | 31762384 | 4.74E−05 | −29.73146408 | Hypo-methylated | NEPC\|CRPC-Adeno DMRs |
| chr19 | 31767694 | 31800060 | 7.82E−14 | −24.8998062 | Hypo-methylated | NEPC\|CRPC-Adeno DMRs |
| chr19 | 31993850 | 32008021 | 0.000835118 | −22.29225699 | Hypo-methylated | NEPC\|CRPC-Adeno DMRs |
| chr19 | 32008765 | 32161906 | 4.14E−08 | −26.13246345 | Hypo-methylated | NEPC\|CRPC-Adeno DMRs |
| chr19 | 32209935 | 32223877 | 0.005737741 | −25.28191335 | Hypo-methylated | NEPC\|CRPC-Adeno DMRs |
| chr19 | 32260602 | 32331169 | 1.71E−06 | −24.31292599 | Hypo-methylated | NEPC\|CRPC-Adeno DMRs |
| chr19 | 32627954 | 32711693 | 0.001694183 | −25.60495808 | Hypo-methylated | NEPC\|CRPC-Adeno DMRs |
| chr19 | 33730345 | 33731533 | 0.005737741 | −33.96332498 | Hypo-methylated | NEPC\|CRPC-Adeno DMRs |
| chr19 | 33883269 | 33884683 | 8.19E−07 | −30.14740175 | Hypo-methylated | NEPC\|CRPC-Adeno DMRs |
| chr19 | 34002335 | 34002501 | 0.001549499 | −20.19802632 | Hypo-methylated | NEPC\|CRPC-Adeno DMRs |
| chr19 | 34175117 | 34175234 | 0.000549237 | 27.03550543 | Hyper-methylated | NEPC\|CRPC-Adeno DMRs |
| chr19 | 34181558 | 34181638 | 0.000549237 | −44.01879699 | Hypo-methylated | NEPC\|CRPC-Adeno DMRs |
| chr19 | 34318594 | 34394363 | 6.16E−05 | −22.55477808 | Hypo-methylated | NEPC\|CRPC-Adeno DMRs |
| chr19 | 34400312 | 34531867 | 1.11E−07 | −28.64637005 | Hypo-methylated | NEPC\|CRPC-Adeno DMRs |
| chr19 | 35793438 | 35793520 | 0.001694183 | −35.21929825 | Hypo-methylated | NEPC\|CRPC-Adeno DMRs |
| chr19 | 36246395 | 36246790 | 8.73E−19 | −26.62591383 | Hypo-methylated | NEPC\|CRPC-Adeno DMRs |
| chr19 | 36319260 | 36319819 | 8.09E−05 | 40.45263158 | Hyper-methylated | NEPC\|CRPC-Adeno DMRs |
| chr19 | 36450599 | 36451091 | 9.76E−10 | −26.8624496 | Hypo-methylated | NEPC\|CRPC-Adeno DMRs |
| chr19 | 36749224 | 36750486 | 0.001310325 | −20.3417401 | Hypo-methylated | NEPC\|CRPC-Adeno DMRs |
| chr19 | 37795602 | 37795949 | 0.007673974 | −22.25140453 | Hypo-methylated | NEPC\|CRPC-Adeno DMRs |
| chr19 | 38042138 | 38042382 | 6.16E−14 | 36.36973328 | Hyper-methylated | NEPC\|CRPC-Adeno DMRs |
| chr19 | 38345759 | 38346574 | 2.08E−05 | 26.977811 | Hyper-methylated | NEPC\|CRPC-Adeno DMRs |
| chr19 | 38527889 | 38528133 | 5.50E−05 | −56.31360318 | Hypo-methylated | NEPC\|CRPC-Adeno DMRs |
| chr19 | 38684237 | 38684378 | 0.000100851 | −58.43795853 | Hypo-methylated | NEPC\|CRPC-Adeno DMRs |
| chr19 | 38851111 | 38851294 | 0.002976353 | −29.48399921 | Hypo-methylated | NEPC\|CRPC-Adeno DMRs |
| chr19 | 38901735 | 38912074 | 0.00013882 | −22.71328772 | Hypo-methylated | NEPC\|CRPC-Adeno DMRs |
| chr19 | 39011057 | 39017921 | 0.000337166 | −28.62690941 | Hypo-methylated | NEPC\|CRPC-Adeno DMRs |
| chr19 | 39034510 | 39053147 | 0.001963239 | −25.42822368 | Hypo-methylated | NEPC\|CRPC-Adeno DMRs |
| chr19 | 39153759 | 39167310 | 5.84E−05 | −26.69099415 | Hypo-methylated | NEPC\|CRPC-Adeno DMRs |
| chr19 | 39193073 | 39193195 | 0.004750844 | −40.32840557 | Hypo-methylated | NEPC\|CRPC-Adeno DMRs |
| chr19 | 39369103 | 39369432 | 0.003327791 | −29.89029605 | Hypo-methylated | NEPC\|CRPC-Adeno DMRs |
| chr19 | 39384074 | 39384230 | 1.55E−06 | −31.79936647 | Hypo-methylated | NEPC\|CRPC-Adeno DMRs |
| chr19 | 39540728 | 39572391 | 0.001549499 | −23.83892988 | Hypo-methylated | NEPC\|CRPC-Adeno DMRs |
| chr19 | 39593570 | 39613396 | 8.38E−05 | −21.01802036 | Hypo-methylated | NEPC\|CRPC-Adeno DMRs |
| chr19 | 39672281 | 39672432 | 0.004750844 | −26.09974937 | Hypo-methylated | NEPC\|CRPC-Adeno DMRs |
| chr19 | 39738370 | 39738859 | 3.07E−11 | −21.84315869 | Hypo-methylated | NEPC\|CRPC-Adeno DMRs |
| chr19 | 39993415 | 39994863 | 3.72E−12 | 32.2470451 | Hyper-methylated | NEPC\|CRPC-Adeno DMRs |
| chr19 | 40344146 | 40362928 | 0.006675101 | −30.93596491 | Hypo-methylated | NEPC\|CRPC-Adeno DMRs |
| chr19 | 40889863 | 40890001 | 5.50E−05 | −24.5374269 | Hypo-methylated | NEPC\|CRPC-Adeno DMRs |
| chr19 | 41116313 | 41116527 | 1.50E−06 | 21.67120743 | Hyper-methylated | NEPC\|CRPC-Adeno DMRs |
| chr19 | 41117037 | 41117285 | 1.35E−06 | −27.25065906 | Hypo-methylated | NEPC\|CRPC-Adeno DMRs |
| chr19 | 41129575 | 41130048 | 0.001274051 | −31.74226608 | Hypo-methylated | NEPC\|CRPC-Adeno DMRs |
| chr19 | 41370990 | 41431809 | 0.003932932 | −21.17071034 | Hypo-methylated | NEPC\|CRPC-Adeno DMRs |
| chr19 | 41494546 | 41571481 | 0.000667554 | −23.14338837 | Hypo-methylated | NEPC\|CRPC-Adeno DMRs |
| chr19 | 41700358 | 41703652 | 0.000522011 | −24.47348887 | Hypo-methylated | NEPC\|CRPC-Adeno DMRs |
| chr19 | 42056914 | 42056948 | 0.001694183 | −28.45438596 | Hypo-methylated | NEPC\|CRPC-Adeno DMRs |
| chr19 | 42436728 | 42437375 | 9.61E−06 | −23.59174641 | Hypo-methylated | NEPC\|CRPC-Adeno DMRs |
| chr19 | 42618590 | 42618828 | 0.000549237 | −45.10927318 | Hypo-methylated | NEPC\|CRPC-Adeno DMRs |
| chr19 | 44203705 | 44203953 | 1.54E−10 | 24.20912281 | Hyper-methylated | NEPC\|CRPC-Adeno DMRs |
| chr19 | 44287281 | 44287377 | 0.001694183 | −24.68703376 | Hypo-methylated | NEPC\|CRPC-Adeno DMRs |
| chr19 | 45198545 | 45200817 | 7.42E−07 | −29.69058845 | Hypo-methylated | NEPC\|CRPC-Adeno DMRs |
| chr19 | 45633604 | 45633814 | 9.09E−05 | −28.81159844 | Hypo-methylated | NEPC\|CRPC-Adeno DMRs |
| chr19 | 45640747 | 45640980 | 7.96E−07 | −32.29637605 | Hypo-methylated | NEPC\|CRPC-Adeno DMRs |
| chr19 | 45992528 | 45996499 | 0.000721524 | 35.4560054 | Hyper-methylated | NEPC\|CRPC-Adeno DMRs |
| chr19 | 46285560 | 46285769 | 0.000209051 | −27.23157895 | Hypo-methylated | NEPC\|CRPC-Adeno DMRs |
| chr19 | 46404991 | 46405120 | 5.34E−09 | −20.14210526 | Hypo-methylated | NEPC\|CRPC-Adeno DMRs |
| chr19 | 47200919 | 47201289 | 0.000832692 | 39.54809942 | Hyper-methylated | NEPC\|CRPC-Adeno DMRs |
| chr19 | 47823993 | 47827068 | 0.000280041 | −25.39675009 | Hypo-methylated | NEPC\|CRPC-Adeno DMRs |
| chr19 | 47844645 | 47845538 | 0.000549237 | −20.19506911 | Hypo-methylated | NEPC\|CRPC-Adeno DMRs |
| chr19 | 47960175 | 47960422 | 3.80E−05 | 21.18215358 | Hyper-methylated | NEPC\|CRPC-Adeno DMRs |
| chr19 | 48285322 | 48285464 | 0.001694183 | 30.73684211 | Hyper-methylated | NEPC\|CRPC-Adeno DMRs |
| chr19 | 48355335 | 48379983 | 2.23E−08 | −25.56577676 | Hypo-methylated | NEPC\|CRPC-Adeno DMRs |
| chr19 | 48699556 | 48699873 | 1.50E−06 | −50.95697454 | Hypo-methylated | NEPC\|CRPC-Adeno DMRs |
| chr19 | 48945846 | 48946375 | 3.33E−15 | 22.94866575 | Hyper-methylated | NEPC\|CRPC-Adeno DMRs |
| chr19 | 49000612 | 49001068 | 6.76E−12 | −24.41349538 | Hypo-methylated | NEPC\|CRPC-Adeno DMRs |
| chr19 | 49183866 | 49183927 | 8.67E−06 | −22.45883442 | Hypo-methylated | NEPC\|CRPC-Adeno DMRs |
| chr19 | 49265604 | 49272230 | 0.005737741 | −22.48480861 | Hypo-methylated | NEPC\|CRPC-Adeno DMRs |
| chr19 | 49368815 | 49373087 | 0.006785531 | −20.66804642 | Hypo-methylated | NEPC\|CRPC-Adeno DMRs |
| chr19 | 49378817 | 49386050 | 5.50E−06 | 22.54897661 | Hyper-methylated | NEPC\|CRPC-Adeno DMRs |
| chr19 | 49724230 | 49756344 | 0.000381376 | −20.85091983 | Hypo-methylated | NEPC\|CRPC-Adeno DMRs |
| chr19 | 50002809 | 50003695 | 0.007379618 | 22.2691416 | Hyper-methylated | NEPC\|CRPC-Adeno DMRs |
| chr19 | 50139374 | 50140426 | 0.001197874 | −24.31226741 | Hypo-methylated | NEPC\|CRPC-Adeno DMRs |
| chr19 | 50204667 | 50204770 | 0.001003946 | 20.24880383 | Hyper-methylated | NEPC\|CRPC-Adeno DMRs |
| chr19 | 50563540 | 50580135 | 2.68E−05 | −27.21026316 | Hypo-methylated | NEPC\|CRPC-Adeno DMRs |
| chr19 | 50691267 | 50693648 | 1.94E−06 | −25.07041562 | Hypo-methylated | NEPC\|CRPC-Adeno DMRs |
| chr19 | 50918132 | 50918386 | 8.35E−06 | 24.5462406 | Hyper-methylated | NEPC\|CRPC-Adeno DMRs |
| chr19 | 50962555 | 50967852 | 0.000721524 | −21.68663968 | Hypo-methylated | NEPC\|CRPC-Adeno DMRs |
| chr19 | 50969644 | 50969782 | 5.50E−05 | −37.0754386 | Hypo-methylated | NEPC\|CRPC-Adeno DMRs |
| chr19 | 51055609 | 51058607 | 5.61E−05 | −27.65397685 | Hypo-methylated | NEPC\|CRPC-Adeno DMRs |
| chr19 | 51072312 | 51106013 | 0.000476559 | −27.03668854 | Hypo-methylated | NEPC\|CRPC-Adeno DMRs |
| chr19 | 51124272 | 51130909 | 0.006890179 | −29.38583496 | Hypo-methylated | NEPC\|CRPC-Adeno DMRs |
| chr19 | 51133255 | 51141334 | 0.00217795 | −31.64432011 | Hypo-methylated | NEPC\|CRPC-Adeno DMRs |
| chr19 | 51144628 | 51156592 | 0.002174767 | −33.41510592 | Hypo-methylated | NEPC\|CRPC-Adeno DMRs |
| chr19 | 51165513 | 51165602 | 0.002976353 | −29.25927318 | Hypo-methylated | NEPC\|CRPC-Adeno DMRs |
| chr19 | 51165693 | 51165766 | 0.001642117 | −27.64736842 | Hypo-methylated | NEPC\|CRPC-Adeno DMRs |
| chr19 | 51171271 | 51171370 | 3.81E−05 | 23.05668016 | Hyper-methylated | NEPC\|CRPC-Adeno DMRs |
| chr19 | 51171495 | 51171543 | 5.17E−06 | 31.94743488 | Hyper-methylated | NEPC\|CRPC-Adeno DMRs |
| chr19 | 51191845 | 51192276 | 2.76E−05 | −29.20917679 | Hypo-methylated | NEPC\|CRPC-Adeno DMRs |
| chr19 | 51199254 | 51207051 | 3.38E−08 | −23.14664099 | Hypo-methylated | NEPC\|CRPC-Adeno DMRs |
| chr19 | 51446863 | 51487774 | 3.59E−09 | −29.89163673 | Hypo-methylated | NEPC\|CRPC-Adeno DMRs |
| chr19 | 51519143 | 51519187 | 0.001694183 | −21.21491228 | Hypo-methylated | NEPC\|CRPC-Adeno DMRs |
| chr19 | 51615497 | 51640963 | 1.55E−07 | −36.34136444 | Hypo-methylated | NEPC\|CRPC-Adeno DMRs |
| chr19 | 51844485 | 51844808 | 1.40E−06 | −23.91701389 | Hypo-methylated | NEPC\|CRPC-Adeno DMRs |
| chr19 | 51873185 | 51892512 | 5.89E−07 | −24.89514772 | Hypo-methylated | NEPC\|CRPC-Adeno DMRs |
| chr19 | 52206187 | 52206908 | 2.64E−09 | −26.15069215 | Hypo-methylated | NEPC\|CRPC-Adeno DMRs |
| chr19 | 52222925 | 52222993 | 0.005737741 | 21.71403509 | Hyper-methylated | NEPC\|CRPC-Adeno DMRs |
| chr19 | 52223076 | 52223159 | 9.54E−05 | 26.38338493 | Hyper-methylated | NEPC\|CRPC-Adeno DMRs |
| chr19 | 53966535 | 53966883 | 1.85E−07 | −20.01984279 | Hypo-methylated | NEPC\|CRPC-Adeno DMRs |
| chr19 | 54101736 | 54126874 | 0.000282282 | −24.1482509 | Hypo-methylated | NEPC\|CRPC-Adeno DMRs |
| chr19 | 54255090 | 54266159 | 2.16E−05 | −27.51664759 | Hypo-methylated | NEPC\|CRPC-Adeno DMRs |
| chr19 | 54291217 | 54337321 | 1.10E−10 | −23.47392545 | Hypo-methylated | NEPC\|CRPC-Adeno DMRs |
| chr19 | 54445326 | 54445403 | 0.001770881 | 20.94207086 | Hyper-methylated | NEPC\|CRPC-Adeno DMRs |
| chr19 | 54466570 | 54466618 | 1.74E−05 | 21.478467 | Hyper-methylated | NEPC\|CRPC-Adeno DMRs |
| chr19 | 54466703 | 54468586 | 0.004705671 | −20.87972222 | Hypo-methylated | NEPC\|CRPC-Adeno DMRs |
| chr19 | 54524742 | 54565132 | 3.34E−06 | −34.04258484 | Hypo-methylated | NEPC\|CRPC-Adeno DMRs |
| chr19 | 54568176 | 54598906 | 5.53E−05 | −24.29076566 | Hypo-methylated | NEPC\|CRPC-Adeno DMRs |
| chr19 | 54926424 | 54926522 | 1.72E−06 | 23.6535731 | Hyper-methylated | NEPC\|CRPC-Adeno DMRs |
| chr19 | 54982501 | 54987554 | 7.38E−13 | −20.41216517 | Hypo-methylated | NEPC\|CRPC-Adeno DMRs |
| chr19 | 55488345 | 55510533 | 1.84E−09 | −24.65547492 | Hypo-methylated | NEPC\|CRPC-Adeno DMRs |
| chr19 | 55602709 | 55602837 | 0.000926684 | −29.91357143 | Hypo-methylated | NEPC\|CRPC-Adeno DMRs |
| chr19 | 55870268 | 55871136 | 5.76E−07 | −41.11788166 | Hypo-methylated | NEPC\|CRPC-Adeno DMRs |
| chr19 | 55944512 | 55948471 | 1.22E−11 | −20.09759004 | Hypo-methylated | NEPC\|CRPC-Adeno DMRs |
| chr19 | 55975812 | 55978467 | 0.000113947 | −20.7416423 | Hypo-methylated | NEPC\|CRPC-Adeno DMRs |
| chr19 | 56061320 | 56066470 | 7.36E−05 | −32.59598301 | Hypo-methylated | NEPC\|CRPC-Adeno DMRs |
| chr19 | 56215437 | 56215478 | 0.001694183 | 20.8489418 | Hyper-methylated | NEPC\|CRPC-Adeno DMRs |
| chr19 | 56292510 | 56308351 | 0.001291252 | −21.05508215 | Hypo-methylated | NEPC\|CRPC-Adeno DMRs |
| chr19 | 56724715 | 56728063 | 0.000889013 | −33.21359649 | Hypo-methylated | NEPC\|CRPC-Adeno DMRs |
| chr19 | 57182985 | 57183206 | 1.47E−06 | 27.41616905 | Hyper-methylated | NEPC\|CRPC-Adeno DMRs |
| chr19 | 57293485 | 57335932 | 8.60E−08 | −23.58802577 | Hypo-methylated | NEPC\|CRPC-Adeno DMRs |
| chr19 | 57618243 | 57618297 | 0.000439429 | 27.90736842 | Hyper-methylated | NEPC\|CRPC-Adeno DMRs |
| chr19 | 58571236 | 58571389 | 0.003348504 | −23.43580247 | Hypo-methylated | NEPC\|CRPC-Adeno DMRs |
| chr19 | 58629408 | 58629979 | 7.27E−07 | 24.03635709 | Hyper-methylated | NEPC\|CRPC-Adeno DMRs |
| chr19 | 59027797 | 59027908 | 0.004750844 | 20.82406015 | Hyper-methylated | NEPC\|CRPC-Adeno DMRs |
| chr20 | 308963 | 309137 | 0.001197874 | 24.49033189 | Hyper-methylated | NEPC\|CRPC-Adeno DMRs |
| chr20 | 644060 | 644131 | 0.004280014 | 21.59537037 | Hyper-methylated | NEPC\|CRPC-Adeno DMRs |
| chr20 | 892978 | 922263 | 0.003327791 | −25.5375 | Hypo-methylated | NEPC\|CRPC-Adeno DMRs |
| chr20 | 941014 | 978552 | 7.73E−06 | −23.42119816 | Hypo-methylated | NEPC\|CRPC-Adeno DMRs |
| chr20 | 982700 | 983022 | 4.48E−08 | 21.1380332 | Hyper-methylated | NEPC\|CRPC-Adeno DMRs |
| chr20 | 1008636 | 1036633 | 0.005239183 | −21.26397967 | Hypo-methylated | NEPC\|CRPC-Adeno DMRs |
| chr20 | 1813231 | 1838648 | 0.000118331 | −22.70699902 | Hypo-methylated | NEPC\|CRPC-Adeno DMRs |
| chr20 | 1874385 | 1874528 | 0.000200845 | 24.53833333 | Hyper-methylated | NEPC\|CRPC-Adeno DMRs |
| chr20 | 1950637 | 1978368 | 2.14E−05 | −28.77314241 | Hypo-methylated | NEPC\|CRPC-Adeno DMRs |
| chr20 | 2384312 | 2397927 | 0.002766856 | −24.13716678 | Hypo-methylated | NEPC\|CRPC-Adeno DMRs |
| chr20 | 2739596 | 2740810 | 0.000667554 | −27.50697368 | Hypo-methylated | NEPC\|CRPC-Adeno DMRs |
| chr20 | 3149092 | 3149321 | 0.000926684 | −20.53947368 | Hypo-methylated | NEPC\|CRPC-Adeno DMRs |
| chr20 | 3635436 | 3635682 | 0.00041315 | −41.81193384 | Hypo-methylated | NEPC\|CRPC-Adeno DMRs |
| chr20 | 3728978 | 3730912 | 0.004665474 | −22.9276477 | Hypo-methylated | NEPC\|CRPC-Adeno DMRs |
| chr20 | 3777367 | 3781531 | 4.69E−08 | −41.84571589 | Hypo-methylated | NEPC\|CRPC-Adeno DMRs |
| chr20 | 4705407 | 4764803 | 0.005772821 | −21.50853523 | Hypo-methylated | NEPC\|CRPC-Adeno DMRs |
| chr20 | 4804547 | 4804841 | 0.002859601 | 27.80504939 | Hyper-methylated | NEPC\|CRPC-Adeno DMRs |
| chr20 | 4836335 | 4836367 | 0.001694183 | 35.55588235 | Hyper-methylated | NEPC\|CRPC-Adeno DMRs |
| chr20 | 5297352 | 5297976 | 1.12E−10 | 23.01575592 | Hyper-methylated | NEPC\|CRPC-Adeno DMRs |
| chr20 | 5594626 | 5594836 | 0.004750844 | 24.15910856 | Hyper-methylated | NEPC\|CRPC-Adeno DMRs |
| chr20 | 10152802 | 10152959 | 8.81E−05 | −20.56667236 | Hypo-methylated | NEPC\|CRPC-Adeno DMRs |
| chr20 | 10198822 | 10200166 | 1.45E−05 | −20.75366357 | Hypo-methylated | NEPC\|CRPC-Adeno DMRs |
| chr20 | 10249807 | 10304164 | 3.15E−05 | 20.77105828 | Hyper-methylated | NEPC\|CRPC-Adeno DMRs |
| chr20 | 10774468 | 10776151 | 2.46E−05 | −25.4325188 | Hypo-methylated | NEPC\|CRPC-Adeno DMRs |
| chr20 | 19192410 | 19192508 | 0.004750844 | −20.06541353 | Hypo-methylated | NEPC\|CRPC-Adeno DMRs |
| chr20 | 19804533 | 19804617 | 5.17E−06 | −21.57511962 | Hypo-methylated | NEPC\|CRPC-Adeno DMRs |
| chr20 | 20180690 | 20232393 | 6.29E−06 | −44.08042265 | Hypo-methylated | NEPC\|CRPC-Adeno DMRs |
| chr20 | 22549081 | 22549680 | 4.86E−11 | 26.5121258 | Hyper-methylated | NEPC\|CRPC-Adeno DMRs |
| chr20 | 22699929 | 22743919 | 0.002226263 | −24.40757009 | Hypo-methylated | NEPC\|CRPC-Adeno DMRs |
| chr20 | 23088974 | 23098317 | 7.00E−05 | −20.21149085 | Hypo-methylated | NEPC\|CRPC-Adeno DMRs |
| chr20 | 23481003 | 23601812 | 0.000446011 | −29.65163711 | Hypo-methylated | NEPC\|CRPC-Adeno DMRs |
| chr20 | 25062860 | 25065114 | 3.57E−12 | 23.11482902 | Hyper-methylated | NEPC\|CRPC-Adeno DMRs |
| chr20 | 29519510 | 29519638 | 0.006785531 | −29.75972222 | Hypo-methylated | NEPC\|CRPC-Adeno DMRs |
| chr20 | 30432637 | 30433410 | 1.28E−08 | 30.82253245 | Hyper-methylated | NEPC\|CRPC-Adeno DMRs |
| chr20 | 31052241 | 31053634 | 0.000263232 | 22.60240416 | Hyper-methylated | NEPC\|CRPC-Adeno DMRs |
| chr20 | 31129334 | 31129483 | 0.00041315 | −48.31754386 | Hypo-methylated | NEPC\|CRPC-Adeno DMRs |
| chr20 | 31211535 | 31216161 | 0.000229476 | −21.30189899 | Hypo-methylated | NEPC\|CRPC-Adeno DMRs |
| chr20 | 32053689 | 32064711 | 0.001694183 | −33.19298246 | Hypo-methylated | NEPC\|CRPC-Adeno DMRs |
| chr20 | 33880930 | 33884334 | 0.005098191 | −23.04328599 | Hypo-methylated | NEPC\|CRPC-Adeno DMRs |
| chr20 | 34004828 | 34009635 | 0.001545937 | −20.35347628 | Hypo-methylated | NEPC\|CRPC-Adeno DMRs |
| chr20 | 34978025 | 34978408 | 0.000153872 | −23.79789474 | Hypo-methylated | NEPC\|CRPC-Adeno DMRs |
| chr20 | 35012302 | 35012357 | 0.009458442 | 31.51998051 | Hyper-methylated | NEPC\|CRPC-Adeno DMRs |
| chr20 | 35441156 | 35441225 | 0.001694183 | 20.3075188 | Hyper-methylated | NEPC\|CRPC-Adeno DMRs |
| chr20 | 36226619 | 36226659 | 0.000549237 | −25.53071518 | Hypo-methylated | NEPC\|CRPC-Adeno DMRs |
| chr20 | 36496319 | 36496400 | 0.005456645 | 21.05943627 | Hyper-methylated | NEPC\|CRPC-Adeno DMRs |
| chr20 | 36923354 | 36924055 | 0.000488426 | −51.75361552 | Hypo-methylated | NEPC\|CRPC-Adeno DMRs |
| chr20 | 36964033 | 36978049 | 3.26E−06 | −26.39951023 | Hypo-methylated | NEPC\|CRPC-Adeno DMRs |
| chr20 | 37303221 | 37303650 | 3.09E−06 | 23.33381144 | Hyper-methylated | NEPC\|CRPC-Adeno DMRs |
| chr20 | 37356911 | 37357223 | 7.13E−09 | 25.31559454 | Hyper-methylated | NEPC\|CRPC-Adeno DMRs |
| chr20 | 42532976 | 42533173 | 0.001065261 | −28.29167527 | Hypo-methylated | NEPC\|CRPC-Adeno DMRs |
| chr20 | 43027683 | 43035902 | 2.55E−05 | −24.07334482 | Hypo-methylated | NEPC\|CRPC-Adeno DMRs |
| chr20 | 44659599 | 44659671 | 0.000549237 | 25.46491228 | Hyper-methylated | NEPC\|CRPC-Adeno DMRs |
| chr20 | 44803094 | 44803290 | 0.000100851 | 24.13308271 | Hyper-methylated | NEPC\|CRPC-Adeno DMRs |
| chr20 | 45946166 | 45946318 | 0.001694183 | −23.05701754 | Hypo-methylated | NEPC\|CRPC-Adeno DMRs |
| chr20 | 48099278 | 48099484 | 8.73E−10 | 51.8393322 | Hyper-methylated | NEPC\|CRPC-Adeno DMRs |
| chr20 | 48626444 | 48626687 | 1.02E−08 | 20.63158755 | Hyper-methylated | NEPC\|CRPC-Adeno DMRs |
| chr20 | 48959019 | 48959154 | 0.000549237 | −40.03773773 | Hypo-methylated | NEPC\|CRPC-Adeno DMRs |
| chr20 | 48995552 | 48996022 | 0.005574264 | −36.97501329 | Hypo-methylated | NEPC\|CRPC-Adeno DMRs |
| chr20 | 49591789 | 49597361 | 0.003113626 | −34.26578947 | Hypo-methylated | NEPC\|CRPC-Adeno DMRs |
| chr20 | 49626842 | 49632122 | 0.000246327 | −26.41781377 | Hypo-methylated | NEPC\|CRPC-Adeno DMRs |
| chr20 | 49656897 | 49749183 | 0.004215557 | −20.75067202 | Hypo-methylated | NEPC\|CRPC-Adeno DMRs |
| chr20 | 49774918 | 49810369 | 9.90E−06 | −26.66495666 | Hypo-methylated | NEPC\|CRPC-Adeno DMRs |
| chr20 | 50045047 | 50045181 | 0.003327791 | 26.32434211 | Hyper-methylated | NEPC\|CRPC-Adeno DMRs |
| chr20 | 50827403 | 51273043 | 9.98E−11 | −28.53140485 | Hypo-methylated | NEPC\|CRPC-Adeno DMRs |
| chr20 | 52293673 | 52314356 | 6.62E−09 | −20.39072134 | Hypo-methylated | NEPC\|CRPC-Adeno DMRs |
| chr20 | 52870107 | 53068788 | 9.35E−05 | −26.63391712 | Hypo-methylated | NEPC\|CRPC-Adeno DMRs |
| chr20 | 53092153 | 53092934 | 3.40E−19 | 28.83178016 | Hyper-methylated | NEPC\|CRPC-Adeno DMRs |
| chr20 | 55019425 | 55032395 | 1.42E−06 | −21.40840613 | Hypo-methylated | NEPC\|CRPC-Adeno DMRs |
| chr20 | 55110369 | 55188960 | 1.54E−06 | −22.64041436 | Hypo-methylated | NEPC\|CRPC-Adeno DMRs |
| chr20 | 55196326 | 55199018 | 0.000581135 | −25.99456976 | Hypo-methylated | NEPC\|CRPC-Adeno DMRs |
| chr20 | 55291980 | 55373228 | 4.10E−17 | −34.89760506 | Hypo-methylated | NEPC\|CRPC-Adeno DMRs |
| chr20 | 55378475 | 55486484 | 1.81E−08 | −31.52491816 | Hypo-methylated | NEPC\|CRPC-Adeno DMRs |
| chr20 | 55503753 | 55580949 | 2.24E−09 | −35.74186142 | Hypo-methylated | NEPC\|CRPC-Adeno DMRs |
| chr20 | 55595661 | 55622875 | 3.54E−05 | −30.7472897 | Hypo-methylated | NEPC\|CRPC-Adeno DMRs |
| chr20 | 55700894 | 55838701 | 8.73E−24 | −27.2291685 | Hypo-methylated | NEPC\|CRPC-Adeno DMRs |
| chr20 | 55849101 | 55924338 | 5.31E−08 | −29.56591269 | Hypo-methylated | NEPC\|CRPC-Adeno DMRs |
| chr20 | 56143552 | 56145310 | 0.003113626 | −30.29561404 | Hypo-methylated | NEPC\|CRPC-Adeno DMRs |
| chr20 | 56160313 | 56167924 | 0.000185101 | −23.22577634 | Hypo-methylated | NEPC\|CRPC-Adeno DMRs |
| chr20 | 56706312 | 56711095 | 0.000549237 | −26.90902256 | Hypo-methylated | NEPC\|CRPC-Adeno DMRs |
| chr20 | 56725858 | 56725950 | 5.01E−05 | −25.8872807 | Hypo-methylated | NEPC\|CRPC-Adeno DMRs |
| chr20 | 57267427 | 57267677 | 0.000749344 | 26.13758822 | Hyper-methylated | NEPC\|CRPC-Adeno DMRs |
| chr20 | 57291744 | 57292353 | 0.000342136 | −38.74633279 | Hypo-methylated | NEPC\|CRPC-Adeno DMRs |
| chr20 | 57370578 | 57376122 | 8.71E−07 | −29.69945878 | Hypo-methylated | NEPC\|CRPC-Adeno DMRs |
| chr20 | 57382431 | 57397852 | 1.35E−06 | −22.96008553 | Hypo-methylated | NEPC\|CRPC-Adeno DMRs |
| chr20 | 57428437 | 57429026 | 0.001310325 | −24.24502924 | Hypo-methylated | NEPC\|CRPC-Adeno DMRs |
| chr20 | 57504269 | 57520234 | 0.003932932 | −36.35698506 | Hypo-methylated | NEPC\|CRPC-Adeno DMRs |
| chr20 | 57875645 | 57875752 | 9.81E−05 | 22.56408283 | Hyper-methylated | NEPC\|CRPC-Adeno DMRs |
| chr20 | 60509158 | 60509203 | 4.34E−06 | −22.0242915 | Hypo-methylated | NEPC\|CRPC-Adeno DMRs |
| chr20 | 60580224 | 60581333 | 0.002976353 | 24.47092732 | Hyper-methylated | NEPC\|CRPC-Adeno DMRs |
| chr20 | 60966906 | 60967143 | 1.94E−06 | −24.07206497 | Hypo-methylated | NEPC\|CRPC-Adeno DMRs |
| chr20 | 60986425 | 60987132 | 0.000128281 | −30.5644615 | Hypo-methylated | NEPC\|CRPC-Adeno DMRs |
| chr20 | 61024219 | 61024433 | 0.00060921 | −24.59098441 | Hypo-methylated | NEPC\|CRPC-Adeno DMRs |
| chr20 | 61028387 | 61033505 | 3.10E−08 | −21.25774926 | Hypo-methylated | NEPC\|CRPC-Adeno DMRs |
| chr20 | 61041025 | 61043756 | 3.41E−09 | −20.40288438 | Hypo-methylated | NEPC\|CRPC-Adeno DMRs |
| chr20 | 61143412 | 61144016 | 0.000337664 | −24.82181287 | Hypo-methylated | NEPC\|CRPC-Adeno DMRs |
| chr20 | 61458886 | 61459302 | 4.69E−05 | −26.69353744 | Hypo-methylated | NEPC\|CRPC-Adeno DMRs |
| chr20 | 61703436 | 61703547 | 0.000443916 | 26.144843 | Hyper-methylated | NEPC\|CRPC-Adeno DMRs |
| chr20 | 61877855 | 61878525 | 0.000244357 | −25.95076401 | Hypo-methylated | NEPC\|CRPC-Adeno DMRs |
| chr20 | 61886650 | 61887454 | 1.15E−05 | −34.24235589 | Hypo-methylated | NEPC\|CRPC-Adeno DMRs |
| chr20 | 61891462 | 61900000 | 8.07E−05 | −21.82449216 | Hypo-methylated | NEPC\|CRPC-Adeno DMRs |
| chr20 | 61947110 | 61959889 | 5.94E−14 | −25.31844096 | Hypo-methylated | NEPC\|CRPC-Adeno DMRs |
| chr20 | 61998337 | 61999044 | 0.001274051 | −29.32428571 | Hypo-methylated | NEPC\|CRPC-Adeno DMRs |
| chr20 | 62037539 | 62037702 | 0.004750844 | −26.71613025 | Hypo-methylated | NEPC\|CRPC-Adeno DMRs |
| chr20 | 62056252 | 62059360 | 4.25E−11 | −23.16045884 | Hypo-methylated | NEPC\|CRPC-Adeno DMRs |
| chr20 | 62069526 | 62069958 | 1.70E−05 | −27.48578947 | Hypo-methylated | NEPC\|CRPC-Adeno DMRs |
| chr20 | 62097963 | 62098052 | 0.000953892 | −24.30838816 | Hypo-methylated | NEPC\|CRPC-Adeno DMRs |
| chr20 | 62133582 | 62134468 | 0.003890964 | −24.77323099 | Hypo-methylated | NEPC\|CRPC-Adeno DMRs |
| chr20 | 62158446 | 62160647 | 0.009285355 | −23.3651302 | Hypo-methylated | NEPC\|CRPC-Adeno DMRs |
| chr20 | 62181232 | 62181658 | 0.000151874 | −22.99764753 | Hypo-methylated | NEPC\|CRPC-Adeno DMRs |
| chr20 | 61499167 | 62499360 | 0.006785531 | 23.60222039 | Hyper-methylated | NEPC\|CRPC-Adeno DMRs |
| chr20 | 61679514 | 62680150 | 2.58E−23 | −27.75461559 | Hypo-methylated | NEPC\|CRPC-Adeno DMRs |
| chr20 | 61700743 | 62700795 | 0.001545937 | −22.1571475 | Hypo-methylated | NEPC\|CRPC-Adeno DMRs |
| chr21 | 9476854 | 9476919 | 0.001694183 | −34.22894737 | Hypo-methylated | NEPC\|CRPC-Adeno DMRs |
| chr21 | 10137563 | 10428163 | 0.002551933 | −27.80873968 | Hypo-methylated | NEPC\|CRPC-Adeno DMRs |
| chr21 | 15077575 | 15077588 | 0.001694183 | 22.94696405 | Hyper-methylated | NEPC\|CRPC-Adeno DMRs |
| chr21 | 15095805 | 15095832 | 0.000176134 | 28.39940476 | Hyper-methylated | NEPC\|CRPC-Adeno DMRs |
| chr21 | 15426939 | 15430513 | 0.000549237 | −26.94847413 | Hypo-methylated | NEPC\|CRPC-Adeno DMRs |
| chr21 | 15443388 | 15448272 | 1.92E−17 | −20.43753804 | Hypo-methylated | NEPC\|CRPC-Adeno DMRs |
| chr21 | 15449797 | 15459405 | 6.19E−08 | −22.49249196 | Hypo-methylated | NEPC\|CRPC-Adeno DMRs |
| chr21 | 18985383 | 18985669 | 0.000187922 | 21.74845483 | Hyper-methylated | NEPC\|CRPC-Adeno DMRs |
| chr21 | 19252484 | 19371261 | 4.10E−05 | −22.0295447 | Hypo-methylated | NEPC\|CRPC-Adeno DMRs |
| chr21 | 16934465 | 26934515 | 0.001694183 | 29.44159111 | Hyper-methylated | NEPC\|CRPC-Adeno DMRs |
| chr21 | 19109178 | 29254156 | 0.003113626 | −38.16666667 | Hypo-methylated | NEPC\|CRPC-Adeno DMRs |
| chr21 | 19157879 | 29636388 | 2.48E−08 | −25.47914469 | Hypo-methylated | NEPC\|CRPC-Adeno DMRs |
| chr21 | 19981165 | 30110926 | 4.66E−05 | −28.95317982 | Hypo-methylated | NEPC\|CRPC-Adeno DMRs |
| chr21 | 34399314 | 34399361 | 0.000732983 | 22.31244019 | Hyper-methylated | NEPC\|CRPC-Adeno DMRs |
| chr21 | 34484111 | 34487710 | 0.005737741 | −20.57017544 | Hypo-methylated | NEPC\|CRPC-Adeno DMRs |
| chr21 | 37816185 | 37832557 | 0.006785531 | −23.97017544 | Hypo-methylated | NEPC\|CRPC-Adeno DMRs |
| chr21 | 38638311 | 38638380 | 0.003525992 | −28.70438596 | Hypo-methylated | NEPC\|CRPC-Adeno DMRs |
| chr21 | 38913493 | 38935585 | 0.000414691 | −32.20423787 | Hypo-methylated | NEPC\|CRPC-Adeno DMRs |
| chr21 | 40031311 | 40032680 | 3.18E−22 | 33.99714482 | Hyper-methylated | NEPC\|CRPC-Adeno DMRs |
| chr21 | 41853755 | 41853981 | 0.009458442 | −23.75877193 | Hypo-methylated | NEPC\|CRPC-Adeno DMRs |
| chr21 | 41798037 | 42798287 | 1.54E−10 | −25.27894737 | Hypo-methylated | NEPC\|CRPC-Adeno DMRs |
| chr21 | 41880155 | 42880727 | 9.24E−06 | 29.09683402 | Hyper-methylated | NEPC\|CRPC-Adeno DMRs |
| chr21 | 43143791 | 43256688 | 1.07E−13 | −27.99569484 | Hypo-methylated | NEPC\|CRPC-Adeno DMRs |
| chr21 | 43441865 | 43442438 | 2.14E−05 | −42.22581126 | Hypo-methylated | NEPC\|CRPC-Adeno DMRs |
| chr21 | 43491949 | 43493461 | 0.006890179 | −35.41474984 | Hypo-methylated | NEPC\|CRPC-Adeno DMRs |
| chr21 | 43714205 | 43714346 | 0.003327791 | −21.10710892 | Hypo-methylated | NEPC\|CRPC-Adeno DMRs |
| chr21 | 43809735 | 43816264 | 0.001694183 | −22.40031605 | Hypo-methylated | NEPC\|CRPC-Adeno DMRs |
| chr21 | 43979021 | 43982996 | 1.55E−07 | −22.93004369 | Hypo-methylated | NEPC\|CRPC-Adeno DMRs |
| chr21 | 44098158 | 44098331 | 0.000549237 | −35.28571429 | Hypo-methylated | NEPC\|CRPC-Adeno DMRs |
| chr11 | 44561049 | 44562122 | 1.98E−05 | −34.22793522 | Hypo-methylated | NEPC\|CRPC-Adeno DMRs |
| chr11 | 44598501 | 44599091 | 0.001694183 | −29.78235867 | Hypo-methylated | NEPC\|CRPC-Adeno DMRs |
| chr21 | 44784605 | 44784739 | 0.000176134 | −28.78267544 | Hypo-methylated | NEPC\|CRPC-Adeno DMRs |
| chr11 | 44787356 | 44787478 | 5.50E−05 | −28.21118755 | Hypo-methylated | NEPC\|CRPC-Adeno DMRs |
| chr11 | 44836765 | 44837407 | 1.74E−08 | −43.07905314 | Hypo-methylated | NEPC\|CRPC-Adeno DMRs |
| chr11 | 44850301 | 44851787 | 2.92E−06 | −42.27993135 | Hypo-methylated | NEPC\|CRPC-Adeno DMRs |
| chr11 | 44871416 | 44872479 | 1.71E−10 | −34.52655229 | Hypo-methylated | NEPC\|CRPC-Adeno DMRs |
| chr11 | 45340509 | 45341350 | 0.000721524 | −21.29966262 | Hypo-methylated | NEPC\|CRPC-Adeno DMRs |
| chr21 | 45356201 | 45356341 | 0.000100851 | −34.05113636 | Hypo-methylated | NEPC\|CRPC-Adeno DMRs |
| chr11 | 45411394 | 45413046 | 5.70E−08 | −22.75322858 | Hypo-methylated | NEPC\|CRPC-Adeno DMRs |
| chr11 | 45590916 | 45591002 | 0.000549237 | 24.87848438 | Hyper-methylated | NEPC\|CRPC-Adeno DMRs |
| chr11 | 45653978 | 45654420 | 5.01E−05 | −49.06045142 | Hypo-methylated | NEPC\|CRPC-Adeno DMRs |
| chr11 | 45698914 | 45699784 | 0.000100851 | −51.81913876 | Hypo-methylated | NEPC\|CRPC-Adeno DMRs |
| chr21 | 45704030 | 45706080 | 6.32E−13 | 24.51650599 | Hyper-methylated | NEPC\|CRPC-Adeno DMRs |
| chr21 | 45770551 | 45770639 | 0.000118331 | 23.39190283 | Hyper-methylated | NEPC\|CRPC-Adeno DMRs |
| chr21 | 45845119 | 45852821 | 1.67E−07 | −21.37780214 | Hypo-methylated | NEPC\|CRPC-Adeno DMRs |
| chr21 | 45881843 | 45887854 | 7.38E−10 | −26.89365863 | Hypo-methylated | NEPC\|CRPC-Adeno DMRs |
| chr21 | 45933491 | 45933891 | 0.000921835 | −36.82032164 | Hypo-methylated | NEPC\|CRPC-Adeno DMRs |
| chr21 | 46107858 | 46107921 | 0.000549237 | −24.79924812 | Hypo-methylated | NEPC\|CRPC-Adeno DMRs |
| chr21 | 46157160 | 46265028 | 0.000538714 | −20.94494285 | Hypo-methylated | NEPC\|CRPC-Adeno DMRs |
| chr21 | 46306516 | 46308476 | 1.35E−06 | −29.01161062 | Hypo-methylated | NEPC\|CRPC-Adeno DMRs |
| chr21 | 46323315 | 46330303 | 3.17E−06 | −22.03156502 | Hypo-methylated | NEPC\|CRPC-Adeno DMRs |
| chr21 | 46336367 | 46338310 | 0.000667554 | −42.5700731 | Hypo-methylated | NEPC\|CRPC-Adeno DMRs |
| chr21 | 46353573 | 46353966 | 4.69E−06 | −31.03906433 | Hypo-methylated | NEPC\|CRPC-Adeno DMRs |
| chr21 | 46404548 | 46406951 | 1.98E−06 | −20.77381971 | Hypo-methylated | NEPC\|CRPC-Adeno DMRs |
| chr21 | 46409981 | 46410237 | 5.75E−05 | −32.99108396 | Hypo-methylated | NEPC\|CRPC-Adeno DMRs |
| chr21 | 46412189 | 46420543 | 4.08E−08 | −30.80796682 | Hypo-methylated | NEPC\|CRPC-Adeno DMRs |
| chr21 | 46421390 | 46422950 | 0.000324528 | −24.85 | Hypo-methylated | NEPC\|CRPC-Adeno DMRs |
| chr21 | 46431972 | 46436249 | 2.71E−05 | −23.58437152 | Hypo-methylated | NEPC\|CRPC-Adeno DMRs |
| chr21 | 46581564 | 46581767 | 0.001694183 | −21.09649123 | Hypo-methylated | NEPC\|CRPC-Adeno DMRs |
| chr21 | 46677446 | 46678543 | 2.78E−13 | −28.46010592 | Hypo-methylated | NEPC\|CRPC-Adeno DMRs |
| chr21 | 46777787 | 46778116 | 0.00302923 | −24.71052632 | Hypo-methylated | NEPC\|CRPC-Adeno DMRs |
| chr21 | 46876301 | 46888880 | 1.47E−30 | −27.53908993 | Hypo-methylated | NEPC\|CRPC-Adeno DMRs |
| chr21 | 46935761 | 46936040 | 0.000291618 | 27.58175876 | Hyper-methylated | NEPC\|CRPC-Adeno DMRs |
| chr21 | 47009971 | 47010272 | 1.95E−10 | 24.70583256 | Hyper-methylated | NEPC\|CRPC-Adeno DMRs |
| chr21 | 47035536 | 47035697 | 0.001694183 | −24.1204793 | Hypo-methylated | NEPC\|CRPC-Adeno DMRs |
| chr21 | 47295095 | 47295140 | 1.70E−05 | 20.46214986 | Hyper-methylated | NEPC\|CRPC-Adeno DMRs |
| chr21 | 47392741 | 47392981 | 1.78E−06 | −21.68457254 | Hypo-methylated | NEPC\|CRPC-Adeno DMRs |
| chr21 | 47405532 | 47409042 | 4.87E−07 | −20.7702046 | Hypo-methylated | NEPC\|CRPC-Adeno DMRs |
| chr21 | 47421891 | 47423680 | 1.14E−11 | −22.47693381 | Hypo-methylated | NEPC\|CRPC-Adeno DMRs |
| chr21 | 47423918 | 47424270 | 0.000176134 | −32.69210526 | Hypo-methylated | NEPC\|CRPC-Adeno DMRs |
| chr21 | 47427051 | 47443808 | 4.14E−06 | −20.4284629 | Hypo-methylated | NEPC\|CRPC-Adeno DMRs |
| chr21 | 47453670 | 47453712 | 0.001694183 | 23.04736842 | Hyper-methylated | NEPC\|CRPC-Adeno DMRs |
| chr21 | 47456828 | 47484922 | 3.21E−15 | −23.81643714 | Hypo-methylated | NEPC\|CRPC-Adeno DMRs |
| chr21 | 47529356 | 47531773 | 1.18E−06 | −31.52359307 | Hypo-methylated | NEPC\|CRPC-Adeno DMRs |
| chr21 | 47556798 | 47561836 | 0.000232488 | −29.38847053 | Hypo-methylated | NEPC\|CRPC-Adeno DMRs |
| chr21 | 47799144 | 47799212 | 4.03E−08 | 22.43387914 | Hyper-methylated | NEPC\|CRPC-Adeno DMRs |
| chr21 | 47813364 | 47813573 | 0.001221669 | −31.41597222 | Hypo-methylated | NEPC\|CRPC-Adeno DMRs |
| chr21 | 47836653 | 47836809 | 8.09E−05 | −24.35706121 | Hypo-methylated | NEPC\|CRPC-Adeno DMRs |
| chr22 | 17081997 | 17083507 | 2.01E−18 | 26.23637974 | Hyper-methylated | NEPC\|CRPC-Adeno DMRs |
| chr22 | 17566412 | 17566990 | 2.11E−12 | 29.53152352 | Hyper-methylated | NEPC\|CRPC-Adeno DMRs |
| chr22 | 17600489 | 17600888 | 5.60E−10 | 24.43126123 | Hyper-methylated | NEPC\|CRPC-Adeno DMRs |
| chr22 | 17679616 | 17679819 | 4.65E−07 | −36.56061298 | Hypo-methylated | NEPC\|CRPC-Adeno DMRs |
| chr22 | 17745256 | 17745813 | 0.002927544 | −32.58235867 | Hypo-methylated | NEPC\|CRPC-Adeno DMRs |
| chr22 | 17795344 | 17795579 | 0.000549237 | −49.48621554 | Hypo-methylated | NEPC\|CRPC-Adeno DMRs |
| chr22 | 17883966 | 17884038 | 0.001694183 | −31.20840991 | Hypo-methylated | NEPC\|CRPC-Adeno DMRs |
| chr22 | 18255268 | 18256402 | 0.005297267 | −20.29991646 | Hypo-methylated | NEPC\|CRPC-Adeno DMRs |
| chr22 | 18291621 | 18291900 | 0.000412306 | −39.32090643 | Hypo-methylated | NEPC\|CRPC-Adeno DMRs |
| chr22 | 18451167 | 18451225 | 0.003525992 | −58.417154 | Hypo-methylated | NEPC\|CRPC-Adeno DMRs |
| chr22 | 19017888 | 19020625 | 0.00213974 | −24.92353801 | Hypo-methylated | NEPC\|CRPC-Adeno DMRs |
| chr22 | 19138290 | 19138349 | 0.000153872 | 25.79181287 | Hv per-methylated | NEPC\|CRPC-Adeno DMRs |
| chr22 | 19171193 | 19172162 | 0.004750844 | −21.87134503 | Hypo-methylated | NEPC\|CRPC-Adeno DMRs |
| chr22 | 19652858 | 19652943 | 5.50E−05 | −22.75230901 | Hypo-methylated | NEPC\|CRPC-Adeno DMRs |
| chr22 | 19799811 | 19799875 | 0.000549237 | 25.43107769 | Hyper-methylated | NEPC\|CRPC-Adeno DMRs |
| chr22 | 19879579 | 19879641 | 0.000549237 | −38.98320951 | Hypo-methylated | NEPC\|CRPC-Adeno DMRs |
| chr22 | 19889511 | 19890171 | 0.000184724 | −48.76962081 | Hypo-methylated | NEPC\|CRPC-Adeno DMRs |
| chr22 | 20142515 | 20142953 | 9.43E−06 | −32.26425439 | Hypo-methylated | NEPC\|CRPC-Adeno DMRs |
| chr22 | 20161417 | 20161962 | 0.002859601 | −35.60833333 | Hypo-methylated | NEPC\|CRPC-Adeno DMRs |
| chr22 | 20220624 | 20220855 | 0.001694183 | −28.91033138 | Hypo-methylated | NEPC\|CRPC-Adeno DMRs |
| chr22 | 20267861 | 20268321 | 0.000101637 | −29.23521767 | Hypo-methylated | NEPC\|CRPC-Adeno DMRs |
| chr22 | 21083590 | 21083979 | 1.09E−05 | −20.23391813 | Hypo-methylated | NEPC\|CRPC-Adeno DMRs |
| chr22 | 22012345 | 22012676 | 8.34E−08 | −21.51363895 | Hypo-methylated | NEPC\|CRPC-Adeno DMRs |
| chr22 | 23244172 | 23267476 | 0.009285355 | −27.44089912 | Hypo-methylated | NEPC\|CRPC-Adeno DMRs |
| chr22 | 24109380 | 24109600 | 0.002976353 | 29.31145572 | Hyper-methylated | NEPC\|CRPC-Adeno DMRs |
| chr22 | 24814938 | 24816978 | 0.000382054 | −22.05648632 | Hypo-methylated | NEPC\|CRPC-Adeno DMRs |
| chr22 | 25081972 | 25082083 | 5.17E−06 | 29.34556087 | Hyper-methylated | NEPC\|CRPC-Adeno DMRs |
| chr22 | 25678822 | 25678981 | 2.19E−08 | 24.54682903 | Hyper-methylated | NEPC\|CRPC-Adeno DMRs |
| chr22 | 26176100 | 26176224 | 0.001694183 | −21.98508772 | Hypo-methylated | NEPC\|CRPC-Adeno DMRs |
| chr22 | 27895126 | 27895313 | 0.000407892 | −21.49534413 | Hypo-methylated | NEPC\|CRPC-Adeno DMRs |
| chr22 | 27900385 | 27900603 | 0.001694183 | −46.3869883 | Hypo-methylated | NEPC\|CRPC-Adeno DMRs |
| chr22 | 28070020 | 28070130 | 0.003475856 | −27.51804511 | Hypo-methylated | NEPC\|CRPC-Adeno DMRs |
| chr22 | 28901886 | 28901942 | 0.001694183 | −38.28274854 | Hypo-methylated | NEPC\|CRPC-Adeno DMRs |
| chr22 | 29811382 | 29811570 | 1.37E−07 | −25.5839972 | Hypo-methylated | NEPC\|CRPC-Adeno DMRs |
| chr22 | 30086484 | 30100682 | 0.00025311 | −34.42611463 | Hypo-methylated | NEPC\|CRPC-Adeno DMRs |
| chr22 | 30601260 | 30601640 | 9.81E−07 | −36.71345029 | Hypo-methylated | NEPC\|CRPC-Adeno DMRs |
| chr22 | 31032454 | 31033046 | 0.000177856 | −25.91453242 | Hypo-methylated | NEPC\|CRPC-Adeno DMRs |
| chr22 | 31218825 | 31218864 | 0.001694183 | 25.16666667 | Hyper-methylated | NEPC\|CRPC-Adeno DMRs |
| chr22 | 32928018 | 32928472 | 0.009458442 | −28.2748538 | Hypo-methylated | NEPC\|CRPC-Adeno DMRs |
| chr22 | 33197453 | 33197838 | 4.12E−16 | 25.54268701 | Hyper-methylated | NEPC\|CRPC-Adeno DMRs |
| chr22 | 34502800 | 34606261 | 0.009285355 | −27.20856661 | Hypo-methylated | NEPC\|CRPC-Adeno DMRs |
| chr22 | 35796905 | 35797018 | 0.007379618 | −20.39766082 | Hypo-methylated | NEPC\|CRPC-Adeno DMRs |
| chr22 | 35937309 | 35938671 | 2.87E−08 | −21.23997156 | Hypo-methylated | NEPC\|CRPC-Adeno DMRs |
| chr22 | 36730276 | 36730344 | 0.001694183 | 41.8742342 | Hyper-methylated | NEPC\|CRPC-Adeno DMRs |
| chr22 | 37302815 | 37308342 | 0.000476227 | −32.00818017 | Hypo-methylated | NEPC\|CRPC-Adeno DMRs |
| chr22 | 37456636 | 37456867 | 5.50E−05 | −20.18518519 | Hypo-methylated | NEPC\|CRPC-Adeno DMRs |
| chr22 | 37736873 | 37740472 | 0.000303685 | −34.93633048 | Hypo-methylated | NEPC\|CRPC-Adeno DMRs |
| chr22 | 37776685 | 37779628 | 0.004750844 | −28.65837212 | Hypo-methylated | NEPC\|CRPC-Adeno DMRs |
| chr22 | 37888713 | 37888806 | 0.000581135 | 28.10307018 | Hyper-methylated | NEPC\|CRPC-Adeno DMRs |
| chr22 | 39549340 | 39549572 | 5.75E−05 | −21.17843881 | Hypo-methylated | NEPC\|CRPC-Adeno DMRs |
| chr22 | 39865530 | 39865971 | 0.000176134 | −42.67516447 | Hypo-methylated | NEPC\|CRPC-Adeno DMRs |
| chr22 | 40042652 | 40043087 | 1.77E−05 | −37.44154705 | Hypo-methylated | NEPC\|CRPC-Adeno DMRs |
| chr22 | 40094351 | 40095658 | 0.001802623 | −44.54761905 | Hypo-methylated | NEPC\|CRPC-Adeno DMRs |
| chr22 | 40419901 | 40424993 | 0.000209051 | 23.93421053 | Hyper-methylated | NEPC\|CRPC-Adeno DMRs |
| chr22 | 40814817 | 40814950 | 0.000488426 | −28.28737353 | Hypo-methylated | NEPC\|CRPC-Adeno DMRs |
| chr22 | 42678710 | 42678986 | 0.007136365 | 31.89947646 | Hyper-methylated | NEPC\|CRPC-Adeno DMRs |
| chr22 | 43511971 | 43521598 | 0.001802623 | 20.44340017 | Hyper-methylated | NEPC\|CRPC-Adeno DMRs |
| chr22 | 43739984 | 43740052 | 2.76E−05 | 30.18724696 | Hyper-methylated | NEPC\|CRPC-Adeno DMRs |
| chr22 | 44893010 | 44893216 | 0.006890179 | −20.19955991 | Hypo-methylated | NEPC\|CRPC-Adeno DMRs |
| chr22 | 44939749 | 44939893 | 0.002976353 | 28.12698413 | Hyper-methylated | NEPC\|CRPC-Adeno DMRs |
| chr22 | 46261813 | 46263196 | 1.20E−10 | 33.14653129 | Hyper-methylated | NEPC\|CRPC-Adeno DMRs |
| chr22 | 46301416 | 46301937 | 2.64E−09 | −37.12364055 | Hypo-methylated | NEPC\|CRPC-Adeno DMRs |
| chr22 | 46307489 | 46307987 | 0.001931256 | −23.88459831 | Hypo-methylated | NEPC\|CRPC-Adeno DMRs |
| chr22 | 46849116 | 46849770 | 5.55E−07 | −20.63170908 | Hypo-methylated | NEPC\|CRPC-Adeno DMRs |
| chr22 | 46859512 | 46859637 | 0.00222033 | −23.01388889 | Hypo-methylated | NEPC\|CRPC-Adeno DMRs |
| chr22 | 46869505 | 46870081 | 0.000488426 | −25.33073424 | Hypo-methylated | NEPC\|CRPC-Adeno DMRs |
| chr22 | 46873741 | 46873999 | 0.001694183 | −30.66081871 | Hypo-methylated | NEPC\|CRPC-Adeno DMRs |
| chr22 | 47003738 | 47003834 | 0.003113626 | 30.23879142 | Hyper-methylated | NEPC\|CRPC-Adeno DMRs |
| chr22 | 47014443 | 47014913 | 5.36E−06 | −39.08979403 | Hypo-methylated | NEPC\|CRPC-Adeno DMRs |
| chr22 | 47048640 | 47049236 | 3.58E−06 | −36.15611492 | Hypo-methylated | NEPC\|CRPC-Adeno DMRs |
| chr22 | 47092338 | 47092652 | 4.34E−06 | −35.36828875 | Hypo-methylated | NEPC\|CRPC-Adeno DMRs |
| chr22 | 47311232 | 47311549 | 0.001694183 | −38.96490532 | Hypo-methylated | NEPC\|CRPC-Adeno DMRs |
| chr22 | 47444995 | 47447605 | 4.20E−05 | −40.47353736 | Hypo-methylated | NEPC\|CRPC-Adeno DMRs |
| chr22 | 47561271 | 47562616 | 1.22E−05 | −46.06201814 | Hypo-methylated | NEPC\|CRPC-Adeno DMRs |
| chr22 | 48902470 | 48903232 | 0.001545937 | −22.72856037 | Hypo-methylated | NEPC\|CRPC-Adeno DMRs |
| chr22 | 49024769 | 49025241 | 8.47E−06 | −23.95748409 | Hypo-methylated | NEPC\|CRPC-Adeno DMRs |
| chr22 | 49096970 | 49097738 | 0.000549237 | −24.15335889 | Hypo-methylated | NEPC\|CRPC-Adeno DMRs |
| chr22 | 50521389 | 50521550 | 0.000176134 | −30.66419956 | Hypo-methylated | NEPC\|CRPC-Adeno DMRs |
| chr22 | 50553152 | 50553314 | 0.000176134 | −36.62126556 | Hypo-methylated | NEPC\|CRPC-Adeno DMRs |
| chr22 | 50681333 | 50681843 | 5.17E−06 | −24.96212121 | Hypo-methylated | NEPC\|CRPC-Adeno DMRs |
| chr22 | 50706027 | 50706503 | 5.78E−15 | 29.97591133 | Hyper-methylated | NEPC\|CRPC-Adeno DMRs |
| chr22 | 50757131 | 50757911 | 0.002989545 | −29.35456064 | Hypo-methylated | NEPC\|CRPC-Adeno DMRs |
| chr22 | 50967984 | 50968018 | 0.001694183 | 30.68412698 | Hyper-methylated | NEPC\|CRPC-Adeno DMRs |
| chr22 | 51143790 | 51144204 | 6.36E−10 | −24.19919468 | Hypo-methylated | NEPC\|CRPC-Adeno DMRs |
| chr22 | 51168048 | 51168130 | 0.000549237 | −37.12180451 | Hypo-methylated | NEPC\|CRPC-Adeno DMRs |
| chrX | 2720584 | 2726281 | 4.29E−06 | −29.85374635 | Hypo-methylated | NEPC\|CRPC-Adeno DMRs |
| chrX | 2773101 | 2779752 | 0.000253274 | −22.22647528 | Hypo-methylated | NEPC\|CRPC-Adeno DMRs |
| chrX | 3264783 | 3264828 | 0.000184724 | 26.86074561 | Hyper-methylated | NEPC\|CRPC-Adeno DMRs |
| chrX | 3643936 | 3659640 | 0.005737741 | −33.6745313 | Hypo-methylated | NEPC\|CRPC-Adeno DMRs |
| chrX | 9175379 | 9175429 | 0.001274051 | 27.68190476 | Hyper-methylated | NEPC\|CRPC-Adeno DMRs |
| chrX | 9984106 | 10006617 | 2.91E−07 | −29.79629462 | Hypo-methylated | NEPC\|CRPC-Adeno DMRs |
| chrX | 14262013 | 14262212 | 2.97E−08 | 23.37515759 | Hyper-methylated | NEPC\|CRPC-Adeno DMRs |
| chrX | 16801615 | 16801690 | 0.000176134 | −22.32534722 | Hypo-methylated | NEPC\|CRPC-Adeno DMRs |
| chrX | 17609935 | 17635448 | 0.00222033 | −40.36451484 | Hypo-methylated | NEPC\|CRPC-Adeno DMRs |
| chrX | 22474666 | 22620452 | 0.004915778 | −22.59945489 | Hypo-methylated | NEPC\|CRPC-Adeno DMRs |
| chrX | 22803914 | 23093737 | 6.76E−11 | −30.25333378 | Hypo-methylated | NEPC\|CRPC-Adeno DMRs |
| chrX | 23106100 | 23329924 | 7.95E−06 | −27.50264284 | Hypo-methylated | NEPC\|CRPC-Adeno DMRs |
| chrX | 23393552 | 23447400 | 5.75E−05 | −32.49538012 | Hypo-methylated | NEPC\|CRPC-Adeno DMRs |
| chrX | 23518215 | 23611170 | 0.000855523 | −20.92451462 | Hypo-methylated | NEPC\|CRPC-Adeno DMRs |
| chrX | 23784306 | 23786342 | 0.001549499 | −27.92872807 | Hypo-methylated | NEPC\|CRPC-Adeno DMRs |
| chrX | 29933339 | 30166275 | 1.97E−06 | −20.48019641 | Hypo-methylated | NEPC\|CRPC-Adeno DMRs |
| chrX | 30672701 | 30697596 | 0.000224358 | 22.3538755 | Hyper-methylated | NEPC\|CRPC-Adeno DMRs |
| chrX | 34960807 | 35813099 | 2.98E−13 | −21.40294562 | Hypo-methylated | NEPC\|CRPC-Adeno DMRs |
| chrX | 37096555 | 37167996 | 0.000542154 | −25.15165186 | Hypo-methylated | NEPC\|CRPC-Adeno DMRs |
| chrX | 37220451 | 37351061 | 0.004750844 | −24.76615497 | Hypo-methylated | NEPC\|CRPC-Adeno DMRs |
| chrX | 37365637 | 37402391 | 0.000601653 | −26.88410176 | Hypo-methylated | NEPC\|CRPC-Adeno DMRs |
| chrX | 37402563 | 37413493 | 1.66E−05 | −26.91652816 | Hypo-methylated | NEPC\|CRPC-Adeno DMRs |
| chrX | 37765122 | 37765701 | 0.000324528 | −20.82286722 | Hypo-methylated | NEPC\|CRPC-Adeno DMRs |
| chrX | 37857688 | 37857750 | 0.001694183 | −40.02777778 | Hypo-methylated | NEPC\|CRPC-Adeno DMRs |
| chrX | 39921467 | 39923700 | 1.55E−06 | −47.82794225 | Hypo-methylated | NEPC\|CRPC-Adeno DMRs |
| chrX | 46937609 | 46938655 | 3.71E−09 | 24.15384888 | Hyper-methylated | NEPC\|CRPC-Adeno DMRs |
| chrX | 47437074 | 47441089 | 0.001197874 | 20.67406964 | Hyper-methylated | NEPC\|CRPC-Adeno DMRs |
| chrX | 48186461 | 48214822 | 0.003113626 | −22.94805478 | Hypo-methylated | NEPC\|CRPC-Adeno DMRs |
| chrX | 48859617 | 48859655 | 0.003525992 | −27.71428571 | Hypo-methylated | NEPC\|CRPC-Adeno DMRs |
| chrX | 50213079 | 50213171 | 0.009285355 | 26.17432383 | Hyper-methylated | NEPC\|CRPC-Adeno DMRs |
| chrX | 50258021 | 50379527 | 0.000104758 | −38.63839009 | Hypo-methylated | NEPC\|CRPC-Adeno DMRs |
| chrX | 53077254 | 53079116 | 5.12E−07 | −21.06639217 | Hypo-methylated | NEPC\|CRPC-Adeno DMRs |
| chrX | 54765487 | 54765561 | 0.001694183 | −26.05214425 | Hypo-methylated | NEPC\|CRPC-Adeno DMRs |
| chrX | 55198670 | 55383234 | 8.14E−05 | −27.14442838 | Hypo-methylated | NEPC\|CRPC-Adeno DMRs |
| chrX | 56264765 | 56406495 | 0.000218704 | −21.48272685 | Hypo-methylated | NEPC\|CRPC-Adeno DMRs |
| chrX | 56407346 | 56532320 | 0.000515039 | −24.26122076 | Hypo-methylated | NEPC\|CRPC-Adeno DMRs |
| chrX | 58293909 | 58296963 | 1.88E−05 | −20.59750576 | Hypo-methylated | NEPC\|CRPC-Adeno DMRs |
| chrX | 64626762 | 64627130 | 1.17E−08 | 22.8738761 | Hyper-methylated | NEPC\|CRPC-Adeno DMRs |
| chrX | 64674766 | 64721864 | 0.002859601 | −27.41517962 | Hypo-methylated | NEPC\|CRPC-Adeno DMRs |
| chrX | 64769732 | 64770733 | 0.000476227 | −26.77719298 | Hypo-methylated | NEPC\|CRPC-Adeno DMRs |
| chrX | 66321282 | 66715762 | 1.27E−06 | −35.79131292 | Hypo-methylated | NEPC\|CRPC-Adeno DMRs |
| chrX | 66785133 | 67238838 | 0.000489849 | −34.76101569 | Hypo-methylated | NEPC\|CRPC-Adeno DMRs |
| chrX | 67679302 | 67696792 | 0.000176134 | −24.54530874 | Hypo-methylated | NEPC\|CRPC-Adeno DMRs |
| chrX | 67885494 | 67907846 | 5.26E−14 | −24.08293411 | Hypo-methylated | NEPC\|CRPC-Adeno DMRs |
| chrX | 68115105 | 68115155 | 0.003932932 | −37.87613301 | Hypo-methylated | NEPC\|CRPC-Adeno DMRs |
| chrX | 68441405 | 68441537 | 0.002094266 | 23.41351875 | Hyper-methylated | NEPC\|CRPC-Adeno DMRs |
| chrX | 68725816 | 68726201 | 7.49E−10 | 31.70154205 | Hyper-methylated | NEPC\|CRPC-Adeno DMRs |
| chrX | 68775663 | 68810730 | 4.58E−05 | −24.2713146 | Hypo-methylated | NEPC\|CRPC-Adeno DMRs |
| chrX | 68892582 | 69307180 | 3.63E−12 | −20.8848458 | Hypo-methylated | NEPC\|CRPC-Adeno DMRs |
| chrX | 69936578 | 70140243 | 0.000220189 | −26.51720795 | Hypo-methylated | NEPC\|CRPC-Adeno DMRs |
| chrX | 70887371 | 70888198 | 0.000484821 | −26.23047429 | Hypo-methylated | NEPC\|CRPC-Adeno DMRs |
| chrX | 71034556 | 71088924 | 0.000400293 | −24.80025585 | Hypo-methylated | NEPC\|CRPC-Adeno DMRs |
| chrX | 71090186 | 71095206 | 8.67E−07 | −34.83030814 | Hypo-methylated | NEPC\|CRPC-Adeno DMRs |
| chrX | 72288762 | 72319796 | 4.76E−08 | −21.21057288 | Hypo-methylated | NEPC\|CRPC-Adeno DMRs |
| chrX | 73755675 | 73756956 | 1.83E−19 | −23.56274177 | Hypo-methylated | NEPC\|CRPC-Adeno DMRs |
| chrX | 74866610 | 74933064 | 0.001694183 | −21.29473684 | Hypo-methylated | NEPC\|CRPC-Adeno DMRs |
| chrX | 77209729 | 77209802 | 0.005737741 | 27.72631579 | Hyper-methylated | NEPC\|CRPC-Adeno DMRs |
| chrX | 77395394 | 78605634 | 0.003525992 | −23.00171967 | Hypo-methylated | NEPC\|CRPC-Adeno DMRs |
| chrX | 78667235 | 79076667 | 1.08E−06 | −40.70439006 | Hypo-methylated | NEPC\|CRPC-Adeno DMRs |
| chrX | 79617823 | 79830328 | 4.44E−09 | −38.70621162 | Hypo-methylated | NEPC\|CRPC-Adeno DMRs |
| chrX | 82763198 | 82763232 | 5.17E−06 | 50.1083865 | Hyper-methylated | NEPC\|CRPC-Adeno DMRs |
| chrX | 90689802 | 90690100 | 7.87E−11 | 25.88303258 | Hyper-methylated | NEPC\|CRPC-Adeno DMRs |
| chrX | 90711585 | 90990226 | 0.000128534 | −23.5898771 | Hypo-methylated | NEPC\|CRPC-Adeno DMRs |
| chrX | 91037116 | 91037166 | 0.007136365 | 32.97593985 | Hyper-methylated | NEPC\|CRPC-Adeno DMRs |
| chrX | 95166938 | 95495115 | 0.000176134 | −33.70090852 | Hypo-methylated | NEPC\|CRPC-Adeno DMRs |
| chrX | 95524551 | 95839408 | 1.41E−05 | −22.52965294 | Hypo-methylated | NEPC\|CRPC-Adeno DMRs |
| chrX | 95882136 | 95898216 | 0.000549237 | −20.42653061 | Hypo-methylated | NEPC\|CRPC-Adeno DMRs |
| chrX | 95949989 | 96082957 | 1.37E−07 | −26.44707049 | Hypo-methylated | NEPC\|CRPC-Adeno DMRs |
| chrX | 100673192 | 100673741 | 1.50E−06 | −25.30832473 | Hypo-methylated | NEPC\|CRPC-Adeno DMRs |
| chrX | 100830003 | 100848342 | 0.001883547 | −27.11148325 | Hypo-methylated | NEPC\|CRPC-Adeno DMRs |
| chrX | 100983206 | 100997072 | 2.76E−05 | −35.76457135 | Hypo-methylated | NEPC\|CRPC-Adeno DMRs |
| chrX | 102475605 | 102475704 | 0.000176134 | 27.50032895 | Hyper-methylated | NEPC\|CRPC-Adeno DMRs |
| chrX | 103463974 | 103495269 | 0.000263522 | −26.50762483 | Hypo-methylated | NEPC\|CRPC-Adeno DMRs |
| chrX | 103498818 | 103499060 | 2.29E−05 | 20.92132783 | Hyper-methylated | NEPC\|CRPC-Adeno DMRs |
| chrX | 104195783 | 105065068 | 3.06E−09 | −40.29809575 | Hypo-methylated | NEPC\|CRPC-Adeno DMRs |
| chrX | 105203773 | 105956548 | 7.18E−11 | −36.16619138 | Hypo-methylated | NEPC\|CRPC-Adeno DMRs |
| chrX | 106567446 | 106568159 | 3.96E−05 | 40.64011925 | Hyper-methylated | NEPC\|CRPC-Adeno DMRs |
| chrX | 108332864 | 108675307 | 4.24E−06 | −38.3554929 | Hypo-methylated | NEPC\|CRPC-Adeno DMRs |
| chrX | 109648004 | 109932415 | 0.000154411 | −30.72858371 | Hypo-methylated | NEPC\|CRPC-Adeno DMRs |
| chrX | 111129539 | 111307181 | 4.65E−07 | −33.14695397 | Hypo-methylated | NEPC\|CRPC-Adeno DMRs |
| chrX | 111356722 | 112022425 | 9.58E−10 | −28.7507352 | Hypo-methylated | NEPC\|CRPC-Adeno DMRs |
| chrX | 112250609 | 113745216 | 4.92E−18 | −30.62801974 | Hypo-methylated | NEPC\|CRPC-Adeno DMRs |
| chrX | 114528373 | 114542387 | 0.000549237 | −25.56691729 | Hypo-methylated | NEPC\|CRPC-Adeno DMRs |
| chrX | 115066914 | 115304521 | 0.003977693 | −26.3951806 | Hypo-methylated | NEPC\|CRPC-Adeno DMRs |
| chrX | 115375230 | 116974824 | 1.40E−12 | −30.59463135 | Hypo-methylated | NEPC\|CRPC-Adeno DMRs |
| chrX | 117411216 | 117411307 | 0.00489825 | 23.83759398 | Hyper-methylated | NEPC\|CRPC-Adeno DMRs |
| chrX | 118234149 | 118284534 | 0.002094266 | −20.02219519 | Hypo-methylated | NEPC\|CRPC-Adeno DMRs |
| chrX | 118827635 | 118827785 | 0.000716333 | −25.97511371 | Hypo-methylated | NEPC\|CRPC-Adeno DMRs |
| chrX | 120135432 | 120135503 | 0.000484821 | 20.00156691 | Hyper-methylated | NEPC\|CRPC-Adeno DMRs |
| chrX | 125299773 | 125299840 | 4.65E−07 | 32.95220423 | Hyper-methylated | NEPC\|CRPC-Adeno DMRs |
| chrX | 125300174 | 125300222 | 0.000176134 | 27.12867647 | Hyper-methylated | NEPC\|CRPC-Adeno DMRs |
| chrX | 125715144 | 125715287 | 1.27E−07 | 25.24954245 | Hyper-methylated | NEPC\|CRPC-Adeno DMRs |
| chrX | 128926390 | 128926468 | 0.000176134 | −42.42127193 | Hypo-methylated | NEPC\|CRPC-Adeno DMRs |
| chrX | 129087400 | 129087488 | 0.00041315 | 34.12610939 | Hyper-methylated | NEPC\|CRPC-Adeno DMRs |
| chrX | 129205782 | 129218327 | 0.00222033 | 24.61198938 | Hyper-methylated | NEPC\|CRPC-Adeno DMRs |
| chrX | 130518133 | 130518205 | 0.000209051 | −31.70398724 | Hypo-methylated | NEPC\|CRPC-Adeno DMRs |
| chrX | 130929727 | 130937281 | 0.000175398 | −20.12668098 | Hypo-methylated | NEPC\|CRPC-Adeno DMRs |
| chrX | 133119214 | 133119361 | 0.005737741 | 20.30547386 | Hyper-methylated | NEPC\|CRPC-Adeno DMRs |
| chrX | 134096722 | 134099911 | 0.000711935 | −30.25339984 | Hypo-methylated | NEPC\|CRPC-Adeno DMRs |
| chrX | 134156102 | 134156681 | 1.44E−11 | −22.37287347 | Hypo-methylated | NEPC\|CRPC-Adeno DMRs |
| chrX | 135294705 | 135301330 | 0.001012154 | −23.23746499 | Hypo-methylated | NEPC\|CRPC-Adeno DMRs |
| chrX | 135332851 | 135333598 | 4.92E−18 | 24.38912671 | Hyper-methylated | NEPC\|CRPC-Adeno DMRs |
| chrX | 135534743 | 135534783 | 0.004750844 | 23.34535481 | Hyper-methylated | NEPC\|CRPC-Adeno DMRs |
| chrX | 135849959 | 135850345 | 0.000101021 | 27.60472348 | Hyper-methylated | NEPC\|CRPC-Adeno DMRs |
| chrX | 136114047 | 136114194 | 0.000101637 | 21.06562703 | Hyper-methylated | NEPC\|CRPC-Adeno DMRs |
| chrX | 136304922 | 136436542 | 0.002376793 | −25.44929938 | Hypo-methylated | NEPC\|CRPC-Adeno DMRs |
| chrX | 136510347 | 136512686 | 1.33E−06 | 21.1966324 | Hyper-methylated | NEPC\|CRPC-Adeno DMRs |
| chrX | 136647741 | 136648946 | 1.79E−11 | 30.26218277 | Hyper-methylated | NEPC\|CRPC-Adeno DMRs |
| chrX | 138066426 | 138259844 | 0.000148603 | −28.48210231 | Hypo-methylated | NEPC\|CRPC-Adeno DMRs |
| chrX | 139013573 | 139013837 | 5.01E−05 | −20.78484405 | Hypo-methylated | NEPC\|CRPC-Adeno DMRs |
| chrX | 139585926 | 139586944 | 1.31E−05 | 23.42806878 | Hyper-methylated | NEPC\|CRPC-Adeno DMRs |
| chrX | 139587125 | 139587898 | 2.35E−05 | 32.79448622 | Hyper-methylated | NEPC\|CRPC-Adeno DMRs |
| chrX | 141479306 | 141577115 | 0.001012154 | −28.0357708 | Hypo-methylated | NEPC\|CRPC-Adeno DMRs |
| chrX | 142723753 | 142723827 | 0.000549237 | 37.68646617 | Hyper-methylated | NEPC\|CRPC-Adeno DMRs |
| chrX | 144712727 | 144713078 | 4.71E−05 | −20.95599243 | Hypo-methylated | NEPC\|CRPC-Adeno DMRs |
| chrX | 144899533 | 144900116 | 1.01E−16 | 24.06204331 | Hyper-methylated | NEPC\|CRPC-Adeno DMRs |
| chrX | 148734113 | 148841487 | 1.90E−05 | −26.55809176 | Hypo-methylated | NEPC\|CRPC-Adeno DMRs |
| chrX | 148971392 | 149022094 | 6.29E−06 | −27.00965132 | Hypo-methylated | NEPC\|CRPC-Adeno DMRs |
| chrX | 149208011 | 149530021 | 0.000489991 | −28.07584598 | Hypo-methylated | NEPC\|CRPC-Adeno DMRs |
| chrX | 150345630 | 150345855 | 3.96E−05 | 28.23133541 | Hyper-methylated | NEPC\|CRPC-Adeno DMRs |
| chrX | 151142874 | 151142959 | 0.000542161 | 23.7860771 | Hyper-methylated | NEPC\|CRPC-Adeno DMRs |
| chrX | 152069090 | 152101505 | 1.11E−09 | −24.79378286 | Hypo-methylated | NEPC\|CRPC-Adeno DMRs |
| chrX | 152662396 | 152663353 | 8.96E−05 | −32.37702068 | Hypo-methylated | NEPC\|CRPC-Adeno DMRs |
| chrX | 152696875 | 152699547 | 0.001694183 | −27.63826754 | Hypo-methylated | NEPC\|CRPC-Adeno DMRs |
| chrX | 152711226 | 152711615 | 8.67E−07 | −39.54041835 | Hypo-methylated | NEPC\|CRPC-Adeno DMRs |
| chrX | 152742281 | 152746419 | 9.82E−05 | −29.59722156 | Hypo-methylated | NEPC\|CRPC-Adeno DMRs |
| chrX | 152747840 | 152752177 | 0.000488426 | −25.04218143 | Hypo-methylated | NEPC\|CRPC-Adeno DMRs |
| chrX | 152767560 | 152767982 | 0.002094266 | −29.39988629 | Hypo-methylated | NEPC\|CRPC-Adeno DMRs |
| chrX | 152785675 | 152792846 | 2.91E−06 | −26.71736998 | Hypo-methylated | NEPC\|CRPC-Adeno DMRs |
| chrX | 152793351 | 152796831 | 0.000721524 | −26.72328498 | Hypo-methylated | NEPC\|CRPC-Adeno DMRs |
| chrX | 152802556 | 152804111 | 0.001555574 | −26.75108843 | Hypo-methylated | NEPC\|CRPC-Adeno DMRs |
| chrX | 152840373 | 152840964 | 0.000209051 | −22.93434148 | Hypo-methylated | NEPC\|CRPC-Adeno DMRs |
| chrX | 152845574 | 152845623 | 9.54E−05 | −29.67856209 | Hypo-methylated | NEPC\|CRPC-Adeno DMRs |
| chrX | 152876129 | 152876287 | 0.001694183 | −22.57135076 | Hypo-methylated | NEPC\|CRPC-Adeno DMRs |
| chrX | 152902993 | 152905638 | 7.44E−06 | −27.43059385 | Hypo-methylated | NEPC\|CRPC-Adeno DMRs |
| chrX | 152958334 | 152958427 | 0.000549237 | −37.45497076 | Hypo-methylated | NEPC\|CRPC-Adeno DMRs |
| chrX | 152960427 | 152971692 | 0.00302923 | 20.7735313 | Hyper-methylated | NEPC\|CRPC-Adeno DMRs |
| chrX | 153053632 | 153054041 | 0.002976353 | −23.40689521 | Hypo-methylated | NEPC\|CRPC-Adeno DMRs |
| chrX | 153127389 | 153127622 | 0.000291618 | 24.02286487 | Hyper-methylated | NEPC\|CRPC-Adeno DMRs |
| chrX | 153171055 | 153171160 | 0.000581135 | −28.11374269 | Hypo-methylated | NEPC\|CRPC-Adeno DMRs |
| chrX | 153215869 | 153215990 | 0.004750844 | 21.00449785 | Hyper-methylated | NEPC\|CRPC-Adeno DMRs |
| chrX | 153381855 | 153382276 | 0.000200845 | −27.32503612 | Hypo-methylated | NEPC\|CRPC-Adeno DMRs |
| chrY | 6132527 | 6720539 | 0.000194541 | −27.9042161 | Hypo-methylated | NEPC\|CRPC-Adeno DMRs |
| chrY | 6787176 | 6790911 | 0.000214872 | −27.03728214 | Hypo-methylated | NEPC\|CRPC-Adeno DMRs |
| chrY | 6790972 | 6954468 | 0.000467288 | −27.11883899 | Hypo-methylated | NEPC\|CRPC-Adeno DMRs |
| chrY | 7525221 | 7645361 | 1.80E−11 | −23.65777201 | Hypo-methylated | NEPC\|CRPC-Adeno DMRs |
| chrY | 8121938 | 8147959 | 1.65E−05 | −27.79421803 | Hypo-methylated | NEPC\|CRPC-Adeno DMRs |
| chrY | 10009069 | 10010109 | 0.002094266 | −24.02090643 | Hypo-methylated | NEPC\|CRPC-Adeno DMRs |
| chrY | 13263288 | 13310760 | 0.002002439 | −20.68611747 | Hypo-methylated | NEPC\|CRPC-Adeno DMRs |
| chrY | 13312337 | 13312912 | 5.69E−07 | −31.49477861 | Hypo-methylated | NEPC\|CRPC-Adeno DMRs |
| chrY | 13314671 | 13328687 | 9.09E−05 | −29.49886878 | Hypo-methylated | NEPC\|CRPC-Adeno DMRs |
| chrY | 14481624 | 14494988 | 0.001694183 | −42.81732804 | Hypo-methylated | NEPC\|CRPC-Adeno DMRs |
| chrY | 14558515 | 14658116 | 0.000669876 | −23.56600754 | Hypo-methylated | NEPC\|CRPC-Adeno DMRs |
| chrY | 14995636 | 15316540 | 6.42E−05 | −25.42790879 | Hypo-methylated | NEPC\|CRPC-Adeno DMRs |
| chrY | 15394674 | 15421396 | 0.000324528 | 22.67280702 | Hyper-methylated | NEPC\|CRPC-Adeno DMRs |
| chrY | 17567474 | 17568277 | 3.49E−15 | −22.13593024 | Hypo-methylated | NEPC\|CRPC-Adeno DMRs |
| chrY | 19155241 | 19472657 | 6.82E−09 | −26.06944582 | Hypo-methylated | NEPC\|CRPC-Adeno DMRs |
| chrY | 21238594 | 21239217 | 1.10E−17 | −24.14056622 | Hypo-methylated | NEPC\|CRPC-Adeno DMRs |
| chrY | 24513436 | 28748362 | 2.59E−06 | −32.66231594 | Hypo-methylated | NEPC\|CRPC-Adeno DMRs |
| chrY | 58990054 | 59033064 | 0.000154148 | −25.62277649 | Hypo-methylated | NEPC\|CRPC-Adeno DMRs |

Interrogation of the end of treatment cases within this cohort (with at least 10% tumor content(Benelli, Romagnoli, and Demichelis (2018) Bioinformatics 34:1642-1649)) revealed similar methylation profile distribution as the CRPC-Adeno cases treated with potent AR pathway inhibitors (FIG. 12D) and the combined set demonstrated significant methylation differences between CRPC-Adeno and CRPC-NE patients (p=0.0031). Notably, the two CRPC-Adeno cases that harbored differentially methylated region (DMR) profiles compatible with CRPC-NE were from patients with visceral metastases as well as other features commonly seen in CRPC-NE (i.e., WCM119 developed radiographic progression on enzalutamide in the setting of a low serum PSA and elevated serum neuroendocrine markers; WCM14 tumor harbored concurrent MYCN amplification and deletion of RB1, which are often enriched in CRPC-NE).

It was found that CRPC-NE is associated with both genomic and epigenomic features that distinguish this resistant subtype from CRPC-Adeno, and these alterations are detectable by ctDNA. While WES and WGBS allowed for the characterization of global patterns and tumor heterogeneity in a patient cohort with relatively high metastatic tumor burden, a molecular classifier for CRPC-NE would be useful if it can be applied at all stages of the disease even in situations with lower tumor burden. This would require a more sensitive targeted assay to run at deeper coverage amenable to lower TC plasma samples. A targeted set of genomic and epigenomic lesions that can identify CRPC-NE was therefore tested using cfDNA. When combining the presence or absence of CRPC-NE-associated features including genomic deletion of mutation of TP53, RB1, and CYLD; lack of mutation or focal gain of AR; and aggregated hypo- and hyper-methylation of 20 differential sites (Table 6) (NEPC feature score, see methods), cfDNA was robust in identifying individuals with CRPC-NE confirmed by histologic analysis of their metastatic biopsy (P=0.000444) (FIG. 12E). Although DNA methylation in itself is a strong classifier of CRPC-NE, it was reasoned that the combination with genomic alterations, likely earlier events, would enable the future identification of high risk cases at earlier stages. These data nominate a ctDNA classifier as a potential biomarker for detecting CRPC-NE non-invasively.

Recent metastatic biopsy studies have uncovered the landscape of genomic and epigenomic alterations enriched in metastatic CRPC compared to primary prostate tumors (Abida et al. (2019) Proc Natl Acad Sci USA 116:11428-11436; Armenia et al. (2018) Nat Genet 50:645-651; Robinson et al. (2015) Cell 161:1215-1228; Quigley et al. (2018) Cell 175:889), yet there is still much to learn regarding the sequence of molecular events that occurs during prostate cancer progression and therapy resistance and the degree of heterogeneity that underlies different stages of the disease. Resistance patterns have largely fallen into two categories: AR-driven and non-AR driven (Watson, Arora, and Sawyers (2015) Nat Rev Cancer 15:701-711). The vast majority of castration resistant tumors are AR-driven, and this can be mediated through AR gene amplification, mutation, splicing, other structural variants, or other means. Specific AR alterations detected by blood sampling, such as AR amplification in ctDNA or AR-V7 splice variant expression in circulating tumor cells, have been associated with poor response and outcomes in patients treated with the AR-targeted drugs abiraterone or enzalutamide (Romanel et al. (2015) Sci Transl Med 7:312re310; Azad et al. (2015) Clin Cancer Res 21:2315-2324; Antonarakis et al. (2014) N Engl J Med 371:1028-1038). This study and others (Romanel et al. (2015) Sci Transl Med 7:312re310; Quigley et al. (2018) Cell 175:889; Viswanathan et al. (2018) Cell 174:433-447.e419; Henzler et al. (2016) Nat Commun 7:13668) support continued evolution of AR gene alterations during disease progression. Non-AR driven disease is less common in CRPC and the mechanisms underlying AR independence are less clear. One increasingly recognized mechanism is the development of lineage plasticity, by which the tumor cells adopt alternative lineage and oncogenic programs to grow despite suppressed AR signaling (Beltran et al. (2019) Clin Cancer Res 25:6916-6924). One extreme manifestation of this is the transformation from a luminal prostate adenocarcinoma to a CRPC-NE phenotype, defined by the acquisition of histologic features similar to small cell lung cancer (Epstein et al. (2014) Am J Surg Pathol 38:756-767) and associated with distinct molecular alterations (Aggarwal et al. (2018) J Clin Oncol JCO2017776880; Benelli, Romagnoli, and Demichelis (2018) Bioinformatics 34:1642-1649). It has been reported that up to 15-20% of patients develop CRPC-NE in late stages of their disease (Abida et al. (2019) Proc Natl Acad Sci USA 116:11428-11436; Aggarwal et al. (2018) J Clin Oncol JCO2017776880; Bluemn et al. (2017) Cancer Cell 32:474-489.e476). Similar to small cell lung cancer and other small cell neuroendocrine carcinomas(9), CRPC-NE is enriched with genomic loss of TP53 and RB/(Abida et al. (2019) Proc Natl Acad Sci USA 116:11428-11436; Benelli, Romagnoli, and Demichelis (2018) Bioinformatics 34:1642-1649; Mu et al. (2017) Science 355:84-88). However, loss of these genes can also occur in non-neuroendocrine prostate cancer (Robinson et al. (2015) Cell 161:1215-1228). In EGFR-mutated lung adenocarcinomas, the presence of TP53 and RB1 loss predisposes patients to later transformation to small cell lung cancer after EGFR inhibitor therapy (Lee et al. (2017) J Clin Oncol 35:3065-3074; Offin et al. (2019) J Thorac Oncol 14:1784-1793). The timing of these events and other molecular alterations during CRPC-NE progression has not been established; the data indicates that they can also be acquired early. DNA methylation and transcriptome analyses have also pointed to specific defining features of CRPC-NE that can improve upon genomics for CRPC-NE disease detection. In the current study, it was found that both genomic and epigenomic features of CRPC-NE are identifiable in patients non-invasively through plasma ctDNA analysis. The diagnosis of CRPC-NE has clinical implications as these patients can be considered for platinum-based chemotherapy (Aparicio et al. (2013) Clin Cancer Res 19:3621-3630; Aparicio et al. (2016) Clin Cancer Res 22:1520-1530) or other non-AR targeted approaches, rather than AR-targeted drugs.

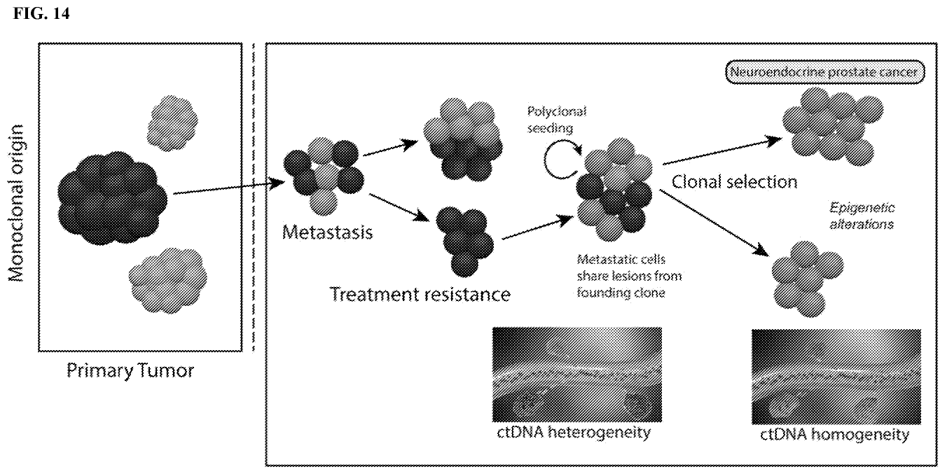

In this study, several prostate cancer alterations shared between both adenocarcinoma and CRPC-NE were also identified, supporting a same cell of origin, though certain genomic alterations (such as AR, TP53, and RB1) had different prognostic value in patients based on their histologic subtype. A genome-wide approach was taken in order to characterize the clonal and subclonal heterogeneity in advanced prostate cancer. Similar to prior studies utilizing targeted DNA or WES (Wyatt et al. (2017) J Natl Cancer Inst 109: doi: 10.1093/jnci/djx118; Adalsteinsson et al. (2017) Nat Commun 8:1324), there was overall high concordance of alterations shared between cfDNA and matched tumor biopsies within individual patients. Allele-specific analysis allowed for a more refined estimation of subclonal diversity. In general, comparison across patients, metastases, and between plasma and biopsies pointed to less heterogeneity in CRPC-NE compared with CRPC-Adeno. This observation, as well as data from published rapid autopsy studies (Kumar et al. (2016) Nat Med 22:369-378; Aryee et al. (2013) Sci Transl Med 5:169ra110), points to higher intra-individual homogeneity across metastases in later stages of the disease. A proposed model of prostate cancer progression towards CRPC-NE building upon published work in the field is depicted in FIG. 14. While prior studies have supported a monoclonal origin of metastatic prostate cancer (Haffner et al. (2013) J Clin Invest 123:4918-4922; Liu et al. (2009) Nat Med 15:559-565) with metastatic lesions often traceable back to a founding clone within the primary tumor (Kumar et al. (2016) Nat Med 22:369-378), tumors do subsequently acquire alterations with disease progression and treatment resistance. Polyclonal spread (Gundem et al. (2015) Nature 520:353-357; Hong et al. (2015) Nat Commun 6:6605) in later stages further leads to intra-patient heterogeneity, particularly of subclonal alterations (as detected here through allele-specific copy number analysis of ctDNA). Evolution of these subclones can contribute to cancer therapy resistance (Burrell and Swanton (2014) Mol Oncol 8:1095-1111; Parikh et al. (2019) Nat Med 25:1415-1421). During the transition towards CRPC-NE and AR independence, the data supports likely selection of a dominant tumor clone (potentially harboring combined loss of RB1 and TP53) that persists and dominates due to selective pressures of therapy resulting in more stringent bottlenecks in the subclonal makeup of the tumor. Therefore, although collective clinical and preclinical data supports CRPC-NE arising through trans-differentiation from a prostate adenocarcinoma precursor, the data also indicates that there can be dominance or clonal selection of a resistant clone during this process, and that these are not two mutually exclusive models of clonal evolution.

It was also found that DNA methylation profiles change with CRPC-NE, which is detectable both in tissues and in cfDNA. DNA methylation patterns have also been reported to be consistent across sites of metastases in men with lethal CRPC-Adeno (Yegnasubramanian et al. (2004) Cancer Res 64:1975-1986; Aryee et al. (2013) Sci Transl Med 5:169ra110), supporting the use of methylation as a relatively stable biomarker for detecting specific resistance patterns. Methylation profiles also point to differential epigenetic regulation of key genes involved in CRPC-NE and can also point to new therapeutic avenues.

While the CRPC-NE subtype was focused on, it was recognized that other resistance subtypes also exist even within non-AR driven disease (Bluemn et al. (2017) Cancer Cell 32:474-489.e476). Nonetheless, these new observations highlight the feasibility and promise of a combined genomic/epigenomic cfDNA approach to identify men with advanced CRPC-NE and provides insights into the degree of intra-patient heterogeneity that exists at different stages of disease progression.

### Example 3: Custom Sequencing Panel

Based on the study results from Beltran et al. (2020) J. Clin. Investigation 130(4):1653-1668, an approach was designed based on a dedicated custom next-generation sequencing panel to quantify the presence of CRPC-Adeno and of CRPC-NE signal in cell-free DNA (cfDNA) of patients with metastatic castrate-resistant prostate cancer (mCRPC). The panel exploits the DNA methylation signal in the circulation as a diagnostic and prognostic biomarker while also providing an estimation of cancer disease burden. This aids the detection of treatment resistance to androgen receptor signaling inhibitors and helps monitoring disease dynamics. In silico data focus on mCRPC, this approach can be extended to earlier disease states.

The custom design stems from a series of computational analyses, including assessment of preliminary performance measures on discovery and test sample sets, and is composed of multiple modules specifically tailored to this precise clinical setting. High-quality data was integrated from public and internal cohorts, including DNA methylation profiles of tissue biopsies and cfDNA from mCRPC patients, white blood cells, and other sources. This procedure allowed for the nomination of a series of genomic regions in which DNA methylation reflects the state of disease. In addition, a knowledge-based approach based on an extensive literature review and the interrogation of gene expression data from both patients and established biological models allowed prioritization of the inclusion of informative sites for the implementation of the ultimate classifiers.

The approach exploits the exquisite tissue specificity of DNA methylation to obtain:

1—A robust estimation of tumor content from cfDNA;

2—A score that captures the evidence of neuroendocrine features and therefore also informative of neuroendocrine treatment resistant phenotype emergence;

3—A score that captures the evidence of CRPC-Adeno features;

4—Additional information through the sequencing profiles of known prostate cancer related genes and regulatory elements.

This multilayered approach is implemented through a targeted strategy that minimize the sequencing cost while retaining sufficient information to achieve the above mentioned tasks.

Detailed information on regions contributing to the multilayered approach for a total of about 100K bp of genomic sequence and the inclusion of about 20K CpG is shown in Table 14.

TABLE 14

| | start_hg19 | end_hg19 | width | reg_id | pval | adjPval | mean_a | mean_b | deltaBeta |
|---|---|---|---|---|---|---|---|---|---|
| chr1 | 32237823 | 32238325 | 503 | 1:32237823-32238325 | 3.3E−45 | 3.02E−42 | 84.4190582 | 1.02922257 | 83.38983566 |
| chr1 | 43814069 | 43814458 | 390 | 1:43814069-43814458 | 6.12E−26 | 3.28E−24 | 9.08307376 | 91.7970836 | −82.7140099 |
| chr1 | 151345023 | 151345348 | 326 | 1:151345023-151345348 | 1.37E−38 | 5.92E−36 | 2.61611719 | 94.2279946 | −91.6118774 |
| chr1 | 151810787 | 151811004 | 218 | 1:151810787-151811004 | 5.31E−50 | 9.36E−47 | 90.3863775 | 0.3447001 | 90.04167737 |
| chr2 | 19561227 | 19561809 | 583 | 2:19561227-19561809 | 3.08E−36 | 9.42E−34 | 90.5382869 | 5.48781566 | 85.05047125 |
| chr2 | 63283706 | 63284232 | 527 | 2:63283706-63284232 | 1.77E−32 | 2.98E−30 | 89.6415386 | 5.5931654 | 84.04837319 |
| chr2 | 177030050 | 177030328 | 279 | 2:177030050-177030328 | 5.74E−30 | 5.69E−28 | 89.9319788 | 5.70881926 | 84.22315955 |
| chr2 | 201450223 | 201450843 | 621 | 2:201450223-201450843 | 5.71E−43 | 4.68E−40 | 86.0864978 | 1.76689758 | 84.31960025 |
| chr3 | 50378091 | 50378764 | 674 | 3:50378091-50378764 | 8.25E−61 | 6.3E−57 | 87.911632 | 0.19797753 | 87.71365445 |
| chr3 | 122296269 | 122296878 | 610 | 3:122296269-122296878 | 6E−42 | 4.04E−39 | 88.3720248 | 1.61277131 | 86.7592535 |
| chr3 | 124860516 | 124860971 | 456 | 3:124860516-124860971 | 3.9E−54 | 1.49E−50 | 88.3411556 | 0.23879672 | 88.10235885 |
| chr5 | 958708 | 959241 | 534 | 5:958708-959241 | 1.18E−35 | 3.39E−33 | 5.85725265 | 88.6846504 | −82.8273977 |
| chr5 | 3187966 | 3188474 | 509 | 5:3187966-3188474 | 2.63E−35 | 7.25E−33 | 9.41259588 | 93.6645583 | −84.2519625 |
| chr5 | 180230667 | 180231294 | 628 | 5:180230667-180231294 | 2.27E−48 | 3.72E−45 | 85.3018416 | 0.5135224 | 84.78831919 |
| chr6 | 13014771 | 13015321 | 551 | 6:13014771-13015321 | 1.31E−23 | 5.02E−22 | 11.0388355 | 91.994453 | −80.9556175 |
| chr6 | 15505245 | 15505560 | 316 | 6:15505245-15505560 | 1.35E−41 | 8.61E−39 | 3.73131251 | 96.2432406 | −92.511928 |
| chr6 | 26271093 | 26271927 | 835 | 6:26271093-26271927 | 2.27E−47 | 2.74E−44 | 88.3044313 | 1.09576558 | 87.2086657 |
| chr7 | 73245339 | 73245852 | 514 | 7:73245339-73245852 | 2.51E−39 | 1.2E−36 | 4.68822041 | 94.1459188 | −89.4576984 |
| chr7 | 129425230 | 129426032 | 803 | 7:129425230-129426032 | 1.77E−45 | 1.77E−42 | 86.066727 | 2.42468618 | 83.64204084 |
| chr8 | 56852048 | 56852390 | 343 | 8:56852048-56852390 | 9.67E−30 | 9.27E−28 | 91.9309828 | 4.96191198 | 86.96907078 |
| chr8 | 99960398 | 99960680 | 283 | 8:99960398-99960680 | 5.34E−40 | 2.91E−37 | 90.5638848 | 2.5593572 | 88.00452764 |
| chr8 | 143625497 | 143626067 | 571 | 8:143625497-143626067 | 3.31E−29 | 2.94E−27 | 10.2304561 | 90.4788259 | −80.2483698 |
| chr8 | 145104006 | 145104459 | 454 | 8:145104006-145104459 | 1.61E−59 | 9.24E−56 | 91.5013281 | 0.83535244 | 90.6659757 |
| chr10 | 6297648 | 6298011 | 364 | 10:6297648-6298011 | 4.83E−34 | 1.1E−31 | 13.2857089 | 96.1793779 | −82.893669 |
| chr10 | 22541895 | 22542245 | 351 | 10:22541895-22542245 | 1.09E−51 | 2.77E−48 | 86.0149881 | 0.74220323 | 85.27278488 |
| chr11 | 393838 | 394389 | 552 | 11:393838-394389 | 4.73E−31 | 5.79E−29 | 6.92757951 | 88.4597663 | −81.5321868 |
| chr11 | 47416258 | 47416635 | 378 | 11:47416258-47416635 | 2.04E−45 | 1.95E−42 | 85.1533209 | 0.72233373 | 84.43098719 |
| chr11 | 63974672 | 63975126 | 455 | 11:63974672-63975126 | 7.04E−53 | 2.31E−49 | 84.9354032 | 0.32106936 | 84.61433388 |
| chr11 | 165408329 | 65408666 | 338 | 11:65408329-65408666 | 2.11E−29 | 1.93E−27 | 91.1600164 | 7.53063149 | 83.62938492 |
| chr11 | 67204996 | 67205391 | 396 | 11:67204996-67205391 | 4.68E−51 | 8.93E−48 | 90.0766068 | 0.50812151 | 89.56848528 |
| chr11 | 69469526 | 69469920 | 395 | 11:69469526-69469920 | 8.21E−39 | 3.69E−36 | 5.57107577 | 93.5603944 | −87.9893186 |
| chr12 | 7060087 | 7060669 | 583 | 12:7060087-7060669 | 1.95E−39 | 9.71E−37 | 86.095922 | 1.93623372 | 84.15968827 |
| chr12 | 53298578 | 53299011 | 434 | 12:53298578-53299011 | 2.88E−33 | 5.69E−31 | 7.02866699 | 90.7201166 | −83.6914496 |
| chr12 | 95941769 | 95942088 | 320 | 12:95941769-95942088 | 3.55E−38 | 1.38E−35 | 87.6771774 | 2.63450911 | 85.04266827 |
| chr12 | 107038917 | 107039245 | 329 | 12:107038917-107039245 | 1.16E−34 | 2.98E−32 | 5.00171857 | 89.9996255 | −84.9979069 |
| chr12 | 124773568 | 124774178 | 611 | 12:124773568-124774178 | 8.36E−28 | 5.97E−26 | 10.386438 | 91.2181357 | −80.8316977 |
| chr12 | 124950569 | 124951090 | 522 | 12:124950569-124951090 | 3.08E−37 | 1.14E−34 | 85.5430995 | 1.76492875 | 83.77817078 |
| chr16 | 1128589 | 1128965 | 377 | 16:1128589-1128965 | 4.4E−51 | 8.93E−48 | 2.74304502 | 82.2920863 | −79.5490413 |
| chr17 | 26732020 | 26732474 | 455 | 17:26732020-26732474 | 2.44E−31 | 3.23E−29 | 13.6142887 | 92.8418976 | −79.227609 |
| chr17 | 41363402 | 41364221 | 820 | 17:41363402-41364221 | 5.44E−65 | 1.25E−60 | 85.2318826 | 0.10935122 | 85.12253134 |
| chr17 | 43318510 | 43319000 | 491 | 17:43318510-43319000 | 7.54E−32 | 1.06E−29 | 8.47998886 | 96.3481809 | −87.868192 |
| chr17 | 79251155 | 79251454 | 300 | 17:79251155-79251454 | 5.25E−36 | 1.54E−33 | 9.46876507 | 97.2975569 | −87.8287919 |
| chr17 | 80358729 | 80358976 | 248 | 17:80358729-80358976 | 1.79E−39 | 9.11E−37 | 92.0105636 | 2.42048361 | 89.59008004 |
| chr17 | 180847037 | 80847370 | 334 | 17:80847037-80847370 | 3.97E−29 | 3.44E−27 | 7.54573671 | 94.2812438 | −86.7355071 |
| chr19 | 2085186 | 2085583 | 398 | 19:2085186-2085583 | 2.11E−51 | 4.82E−48 | 83.7622241 | 0.18402373 | 83.57820034 |
| chr19 | 2588141 | 2588729 | 589 | 19:2588141-2588729 | 2.43E−42 | 1.83E−39 | 8.13064945 | 92.8745326 | −84.7438832 |
| chr19 | 3179264 | 3179928 | 665 | 19:3179264-3179928 | 5.07E−47 | 5.27E−44 | 89.4061144 | 0.85930829 | 88.54680613 |
| chr19 | 10735906 | 10736548 | 643 | 19:10735906-10736548 | 2.81E−47 | 3.06E−44 | 1.61212215 | 86.9383503 | −85.3262281 |
| chr19 | 15568260 | 15568674 | 415 | 19:15568260-15568674 | 1.97E−38 | 8.22E−36 | 8.66701571 | 96.9957427 | −88.328727 |
| chr19 | 18872937 | 18873368 | 432 | 19:18872937-18873368 | 5.93E−37 | 2.06E−34 | 6.34442173 | 87.9339882 | −81.5895664 |
| chr19 | 35940325 | 35940693 | 369 | 19:35940325-35940693 | 9.41E−43 | 7.44E−40 | 3.6539835 | 88.2941749 | −84.6401914 |
| chr19 | 42703509 | 42703952 | 444 | 19:42703509-42703952 | 1.35E−52 | 3.87E−49 | 88.8709441 | 0.71022877 | 88.16071533 |
| chr22 | 48027541 | 48028021 | 481 | 22:48027541-48028021 | 2.78E−30 | 2.99E−28 | 10.9544553 | 93.264643 | −82.3101877 |
| chr1 | 2036183 | 2036743 | 561 | 1:2036183-2036743 | 1.33E−07 | 1.88E−05 | 29.2974765 | 79.8883233 | −50.5908468 |
| chr1 | 2309984 | 2310427 | 444 | 1:2309984-2310427 | 0.000292 | 0.001931 | 42.698635 | 82.5602053 | −39.8615703 |
| chr1 | 2379650 | 2380037 | 388 | 1:2379650-2380037 | 1.28E−05 | 0.000245 | 33.2831441 | 71.2324977 | −37.9493536 |
| chr1 | 9887711 | 9888084 | 374 | 1:9887711-9888084 | 1.22E−06 | 6.41E−05 | 31.1428061 | 84.5949469 | −53.4521408 |
| chr1 | 119870187 | 119870578 | 392 | 1:119870187-119870578 | 2.37E−06 | 9.05E−05 | 11.9602888 | 48.2547951 | −36.2945063 |
| chr1 | 150122937 | 150123246 | 310 | 1:150122937-150123246 | 1.3E−09 | 2.83E−06 | 1.1592797 | 44.0592691 | −42.8999894 |
| chr1 | 155034908 | 155035630 | 723 | 1:155034908-155035630 | 3.04E−05 | 0.000428 | 22.0977443 | 56.2498919 | −34.1521476 |
| chr1 | 156215318 | 156215827 | 510 | 1:156215318-156215827 | 1.62E−05 | 0.000283 | 10.4301554 | 41.6367403 | −31.2065849 |
| chr1 | 205631878 | 205632201 | 324 | 1:205631878-205632201 | 1.85E−05 | 0.00031 | 84.6472237 | 48.1652722 | 36.48195151 |
| chr1 | 234666987 | 234667649 | 663 | 1:234666987-234667649 | 2.84E−06 | 0.000102 | 76.1895099 | 32.0130393 | 44.17647065 |
| chr1 | 234908126 | 234908614 | 489 | 1:234908126-234908614 | 8.06E−07 | 5.02E−05 | 10.9611526 | 54.6456179 | −43.6844653 |
| chr1 | 235805303 | 235805870 | 568 | 1:235805303-235805870 | 1.79E−05 | 0.000303 | 17.9690749 | 59.7671146 | −41.7980397 |
| chr1 | 246939116 | 246939599 | 484 | 1:246939116-246939599 | 7.52E−08 | 1.44E−05 | 83.0509744 | 31.0864904 | 51.96448403 |
| chr2 | 3281230 | 3281630 | 401 | 2:3281230-3281630 | 2.24E−07 | 2.4E−05 | 43.2963744 | 84.8479269 | −41.5515525 |
| chr2 | 10567426 | 10567943 | 518 | 2:10567426-10567943 | 6.61E−09 | 5.18E−06 | 83.6971494 | 40.8015222 | 42.89562721 |
| chr2 | 11796686 | 11797117 | 432 | 2:11796686-11797117 | 0.000813 | 0.003895 | 59.0405326 | 28.4723577 | 30.56817493 |
| chr2 | 45237589 | 45238029 | 441 | 2:45237589-45238029 | 2.75E−05 | 0.000403 | 19.4306646 | 53.108711 | −33.6780464 |
| chr2 | 121432974 | 121433398 | 425 | 2:121432974-121433398 | 3.14E−05 | 0.000437 | 54.5778497 | 21.160545 | 33.41730464 |
| chr2 | 172952783 | 172953730 | 948 | 2:172952783-172953730 | 0.000318 | 0.002051 | 62.294041 | 17.9756988 | 44.31834221 |
| chr2 | 240161519 | 240162024 | 506 | 2:240161519-240162024 | 4.91E−05 | 0.000583 | 44.2505809 | 88.9239685 | −44.6733876 |
| chr3 | 134125473 | 134125970 | 498 | 3:134125473-134125970 | 2.58E−05 | 0.000387 | 14.9049696 | 46.5163818 | −31.6114122 |
| chr3 | 140813669 | 140814233 | 565 | 3:140813669-140814233 | 2.33E−08 | 7.9E−06 | 12.7684241 | 73.9847391 | −61.216315 |
| chr3 | 160167217 | 160168090 | 874 | 3:160167217-160168090 | 8.06E−07 | 5.02E−05 | 62.0404129 | 27.7345036 | 34.30590926 |
| chr3 | 183891828 | 183892160 | 333 | 3:183891828-183892160 | 2.12E−06 | 8.55E−05 | 78.1346887 | 46.0142015 | 32.12048723 |
| chr1 | 981385 | 981730 | 346 | 4:981385-981730 | 2.12E−05 | 0.000338 | 57.7473983 | 25.158259 | 32.58913935 |
| chr1 | 1205814 | 1206250 | 437 | 4:1205814-1206250 | 3.45E−07 | 3.11E−05 | 69.7960814 | 23.6607424 | 46.13533894 |
| chr1 | 7228168 | 7228542 | 375 | 4:7228168-7228542 | 1.49E−09 | 2.83E−06 | 16.0630753 | 67.7153432 | −51.652268 |
| chr5 | 92938049 | 92938502 | 454 | 5:92938049-92938502 | 2.29E−06 | 8.93E−05 | 22.0961037 | 70.1667547 | −48.070651 |
| chr5 | 134827036 | 134827920 | 885 | 5:134827036-134827920 | 8.7E−07 | 5.23E−05 | 18.7657868 | 67.4697531 | −48.7039663 |
| chr5 | 147162061 | 147162561 | 501 | 5:147162061-147162561 | 9.02E−07 | 5.27E−05 | 9.98210836 | 45.9661431 | −35.9840348 |
| chr5 | 179588340 | 179588938 | 599 | 5:179588340-179588938 | 1.5E−07 | 1.99E−05 | 21.0918301 | 68.7926482 | −47.7008181 |
| chr6 | 40567217 | 40567679 | 463 | 6:40567217-40567679 | 3.32E−07 | 3.06E−05 | 82.0148388 | 43.1867291 | 38.8281097 |
| chr6 | 170449317 | 170449687 | 371 | 6:170449317-170449687 | 3.79E−06 | 0.000118 | 25.3920229 | 79.4525391 | −54.0605162 |
| chr6 | 170578492 | 170578955 | 464 | 6:170578492-170578955 | 4.88E−10 | 2.83E−06 | 10.4978228 | 81.5550674 | −71.0572446 |
| chr7 | 214477 | 214879 | 403 | 7:214477-214879 | 2.29E−06 | 8.93E−05 | 78.1328361 | 40.453918 | 37.67891814 |
| chr7 | 906626 | 906899 | 274 | 7:906626-906899 | 0.000571 | 0.003063 | 64.4067577 | 20.7767645 | 43.62999316 |
| chr7 | 921951 | 922335 | 385 | 7:921951-922335 | 1.97E−06 | 8.27E−05 | 81.926409 | 48.8087809 | 33.11762811 |
| chr7 | 2968216 | 2968727 | 512 | 7:2968216-2968727 | 4.24E−10 | 2.83E−06 | 12.4490208 | 82.2567882 | −69.8077674 |
| chr7 | 50628618 | 50629167 | 550 | 7:50628618-50629167 | 9.39E−09 | 6.09E−06 | 13.1582853 | 60.7615549 | −47.6032696 |
| chr7 | 129912387 | 129912903 | 517 | 7:129912387-129912903 | 7.52E−08 | 1.44E−05 | 18.6106584 | 66.2496103 | −47.6389519 |
| chr7 | 151432920 | 151433426 | 507 | 7:151432920-151433426 | 5.5E−07 | 3.96E−05 | 23.0478882 | 74.3771788 | −51.3292906 |
| chr8 | 73848837 | 73849214 | 378 | 8:73848837-73849214 | 2.7E−09 | 3.78E−06 | 91.0293796 | 39.9627044 | 51.0666752 |
| chr8 | 93114050 | 93114537 | 488 | 8:93114050-93114537 | 1.64E−06 | 7.52E−05 | 10.1175034 | 43.285464 | −33.1679606 |
| chr10 | 670687 | 671107 | 421 | 10:670687-671107 | 1.56E−07 | 2.04E−05 | 11.3398594 | 53.3785278 | −42.0386684 |
| chr10 | 123923781 | 123924185 | 405 | 10:123923781-123924185 | 0.004144 | 0.012576 | 39.9919206 | 71.052937 | −31.0610164 |
| chr11 | 20625892 | 20626364 | 473 | 11:20625892-20626364 | 1.83E−06 | 7.92E−05 | 71.0769777 | 32.0873133 | 38.98966441 |
| chr11 | 61449637 | 61449998 | 362 | 11:61449637-61449998 | 0.000132 | 0.001122 | 31.1797301 | 77.8045169 | −46.6247868 |
| chr11 | 62211890 | 62212295 | 406 | 11:62211890-62212295 | 0.001271 | 0.005377 | 28.3755331 | 59.1119954 | −30.7364623 |
| chr12 | 104852255 | 104852546 | 292 | 12:104852255-104852546 | 2.05E−06 | 8.38E−05 | 15.570431 | 57.1846334 | −41.6142024 |
| chr12 | 110172108 | 110172446 | 339 | 12:110172108-110172446 | 0.000346 | 0.002169 | 31.540024 | 64.1181487 | −32.5781248 |
| chr12 | 125578255 | 125578645 | 391 | 12:125578255-125578645 | 2.07E−07 | 2.34E−05 | 31.0194084 | 89.3987285 | −58.3793201 |
| chr14 | 91862713 | 91863007 | 295 | 14:91862713-91863007 | 0.002971 | 0.009904 | 51.8932283 | 19.3793181 | 32.51391017 |
| chr14 | 95693684 | 95694081 | 398 | 14:95693684-95694081 | 1.91E−07 | 2.2E−05 | 15.6325476 | 67.1341886 | −51.5016411 |
| chr14 | 101157815 | 101158351 | 537 | 14:101157815-101158351 | 1.42E−06 | 6.92E−05 | 25.5436473 | 61.9343844 | −36.3907372 |
| chr15 | 89921572 | 89922171 | 600 | 15:89921572-89922171 | 0.000336 | 0.002132 | 55.8796079 | 8.54258924 | 47.33701869 |
| chr15 | 89959557 | 89960378 | 822 | 15:89959557-89960378 | 4.54E−07 | 3.58E−05 | 52.3302336 | 19.559138 | 32.77109557 |
| chr15 | 99194331 | 99194724 | 394 | 15:99194331-99194724 | 0.002387 | 0.008462 | 47.5355992 | 14.2538722 | 33.28172704 |
| chr16 | 1195273 | 1195746 | 474 | 16:1195273-1195746 | 9.41E−10 | 2.83E−06 | 15.0972294 | 63.1887484 | −48.091519 |
| chr16 | 29324459 | 29324913 | 455 | 16:29324459-29324913 | 1.91E−07 | 2.2E−05 | 38.3473588 | 88.4580919 | −50.1107331 |
| chr17 | 59480574 | 59481380 | 807 | 17:59480574-59481380 | 4.71E−07 | 3.61E−05 | 15.7907982 | 60.8904821 | −45.0996838 |
| chr17 | 60827102 | 60827625 | 524 | 17:60827102-60827625 | 0.000225 | 0.001614 | 57.8105271 | 89.3244045 | −31.5138774 |
| chr17 | 71744333 | 71745024 | 692 | 17:71744333-71745024 | 1.03E−09 | 2.83E−06 | 22.1380107 | 81.0269779 | −58.8889672 |
| chr17 | 72350254 | 72350810 | 557 | 17:72350254-72350810 | 1.37E−05 | 0.000256 | 62.7630357 | 26.2619516 | 36.50108413 |
| chr17 | 73996145 | 73996523 | 379 | 17:73996145-73996523 | 6.9E−06 | 0.000168 | 70.8872775 | 37.6492854 | 33.23799205 |
| chr17 | 76355046 | 76355388 | 343 | 17:76355046-76355388 | 0.000498 | 0.00278 | 27.5581355 | 62.1679704 | −34.6098349 |
| chr17 | 78079381 | 78079895 | 515 | 17:78079381-78079895 | 3.27E−08 | 8.85E−06 | 15.8798978 | 70.7792498 | −54.8993519 |
| chr17 | 79223965 | 79224406 | 442 | 17:79223965-79224406 | 2.83E−09 | 3.78E−06 | 29.6680035 | 90.8854369 | −61.2174334 |
| chr17 | 80019426 | 80019727 | 302 | 17:80019426-80019727 | 1.58E−06 | 7.36E−05 | 75.0798437 | 35.4526326 | 39.62721109 |
| chr17 | 80052807 | 80053305 | 499 | 17:80052807-80053305 | 2.37E−06 | 9.05E−05 | 84.0415206 | 43.8948243 | 40.14669627 |
| chr17 | 80372675 | 80373012 | 338 | 17:80372675-80373012 | 3E−08 | 8.54E−06 | 66.1646352 | 10.4791332 | 55.68550201 |
| chr17 | 80832228 | 80832711 | 484 | 17:80832228-80832711 | 9.46E−05 | 0.000908 | 72.755824 | 41.7060042 | 31.04981985 |
| chr18 | 8659409 | 8660264 | 856 | 18:8659409-8660264 | 6.66E−07 | 4.43E−05 | 40.944379 | 90.3811125 | −49.4367336 |
| chr18 | 13136240 | 13136697 | 458 | 18:13136240-13136697 | 1.16E−05 | 0.000231 | 16.2504508 | 48.9326406 | −32.6821899 |
| chr19 | 2540807 | 2541204 | 398 | 19:2540807-2541204 | 0.001683 | 0.006621 | 55.4535572 | 25.359578 | 30.09397919 |
| chr19 | 4566300 | 4566777 | 478 | 19:4566300-4566777 | 1.31E−06 | 6.67E−05 | 25.1844671 | 67.8480667 | −42.6635996 |
| chr19 | 17790974 | 17791454 | 481 | 19:17790974-17791454 | 4.03E−08 | 1.01E−05 | 66.6891879 | 26.8897241 | 39.79946375 |
| chr20 | 20432962 | 20433428 | 467 | 20:20432962-20433428 | 3.28E−06 | 0.000109 | 21.392767 | 70.3561572 | −48.9633902 |
| chr20 | 51589516 | 51590085 | 570 | 20:51589516-51590085 | 5.87E−08 | 1.24E−05 | 6.85074047 | 46.1888636 | −39.3381231 |
| chr21 | 38077158 | 38077773 | 616 | 21:38077158-38077773 | 8.63E−05 | 0.000854 | 60.4447787 | 23.6681135 | 36.77666515 |
| chr21 | 43441927 | 43442354 | 428 | 21:43441927-43442354 | 4.05E−09 | 4.61E−06 | 22.3652428 | 78.5517232 | −56.1864805 |
| chr1 | 2036220 | 2036223 | 4 | 1:2036220-2036223 | 7.48E−07 | 3.62E−06 | 21.9190476 | 66.0942774 | −44.1752297 |
| chr1 | 2036226 | 2036229 | 4 | 1:2036226-2036229 | 7.48E−07 | 3.62E−06 | 21.9190476 | 66.0942774 | −44.1752297 |
| chr1 | 2036232 | 2036235 | 4 | 1:2036232-2036235 | 7.48E−07 | 3.62E−06 | 21.9190476 | 66.0942774 | −44.1752297 |
| chr1 | 2036252 | 2036255 | 4 | 1:2036252-2036255 | 7.48E−07 | 3.62E−06 | 21.9190476 | 66.0942774 | −44.1752297 |
| chr1 | 2036395 | 2036398 | 4 | 1:2036395-2036398 | 7.48E−07 | 3.62E−06 | 21.9190476 | 66.0942774 | −44.1752297 |
| chr1 | 3697617 | 3697620 | 4 | 1:3697617-3697620 | 1.83E−06 | 7.94E−06 | 81.3854701 | 36.7501654 | 44.6353047 |
| chr1 | 3697629 | 3697634 | 6 | 1:3697629-3697634 | 1.83E−06 | 7.94E−06 | 81.3854701 | 36.7501654 | 44.6353047 |
| chr1 | 3697639 | 3697642 | 4 | 1:3697639-3697642 | 1.83E−06 | 7.94E−06 | 81.3854701 | 36.7501654 | 44.6353047 |
| chr1 | 3697648 | 3697651 | 4 | 1:3697648-3697651 | 1.83E−06 | 7.94E−06 | 81.3854701 | 36.7501654 | 44.6353047 |
| chr1 | 3697654 | 3697661 | 8 | 1:3697654-3697661 | 1.83E−06 | 7.94E−06 | 81.3854701 | 36.7501654 | 44.6353047 |
| chr1 | 3697667 | 3697670 | 4 | 1:3697667-3697670 | 1.83E−06 | 7.94E−06 | 81.3854701 | 36.7501654 | 44.6353047 |
| chr1 | 3697692 | 3697695 | 4 | 1:3697692-3697695 | 1.83E−06 | 7.94E−06 | 81.3854701 | 36.7501654 | 44.6353047 |
| chr1 | 3697702 | 3697705 | 4 | 1:3697702-3697705 | 1.83E−06 | 7.94E−06 | 81.3854701 | 36.7501654 | 44.6353047 |
| chr1 | 6171653 | 6171656 | 4 | 1:6171653-6171656 | 8.17E−10 | 7.54E−09 | 17.332906 | 60.0073887 | −42.6744827 |
| chr1 | 6171833 | 6171836 | 4 | 1:6171833-6171836 | 8.17E−10 | 7.54E−09 | 17.332906 | 60.0073887 | −42.6744827 |
| chr1 | 6171838 | 6171841 | 4 | 1:6171838-6171841 | 8.17E−10 | 7.54E−09 | 17.332906 | 60.0073887 | −42.6744827 |
| chr1 | 6171845 | 6171848 | 4 | 1:6171845-6171848 | 8.17E−10 | 7.54E−09 | 17.332906 | 60.0073887 | −42.6744827 |
| chr1 | 6171867 | 6171873 | 7 | 1:6171867-6171873 | 8.17E−10 | 7.54E−09 | 17.332906 | 60.0073887 | −42.6744827 |
| chr1 | 9501648 | 9501651 | 4 | 1:9501648-9501651 | 7.77E−05 | 0.000217 | 28.9487103 | 74.1431674 | −45.194457 |
| chr1 | 10696021 | 10696025 | 5 | 1:10696021-10696025 | 3.3E−14 | 6.43E−13 | 64.2131579 | 21.9722992 | 42.24085873 |
| chr1 | 10696065 | 10696068 | 4 | 1:10696065-10696068 | 3.3E−14 | 6.43E−13 | 64.2131579 | 21.9722992 | 42.24085873 |
| chr1 | 18808323 | 18808326 | 4 | 1:18808323-18808326 | 1.64E−40 | 6.63E−38 | 36.7727472 | 90.0271183 | −53.2543711 |
| chr1 | 18808343 | 18808346 | 4 | 1:18808343-18808346 | 1.64E−40 | 6.63E−38 | 36.7727472 | 90.0271183 | −53.2543711 |
| chr1 | 18808439 | 18808442 | 4 | 1:18808439-18808442 | 1.64E−40 | 6.63E−38 | 36.7727472 | 90.0271183 | −53.2543711 |
| chr1 | 18808469 | 18808472 | 4 | 1:18808469-18808472 | 1.64E−40 | 6.63E−38 | 36.7727472 | 90.0271183 | −53.2543711 |
| chr1 | 18808546 | 18808549 | 4 | 1:18808546-18808549 | 1.64E−40 | 6.63E−38 | 36.7727472 | 90.0271183 | −53.2543711 |
| chr1 | 18808583 | 18808586 | 4 | 1:18808583-18808586 | 1.64E−40 | 6.63E−38 | 36.7727472 | 90.0271183 | −53.2543711 |
| chr1 | 18808621 | 18808624 | 4 | 1:18808621-18808624 | 1.64E−40 | 6.63E−38 | 36.7727472 | 90.0271183 | −53.2543711 |
| chr1 | 18808832 | 18808835 | 4 | 1:18808832-18808835 | 1.64E−40 | 6.63E−38 | 36.7727472 | 90.0271183 | −53.2543711 |
| chr1 | 18808880 | 18808883 | 4 | 1:18808880-18808883 | 1.64E−40 | 6.63E−38 | 36.7727472 | 90.0271183 | −53.2543711 |
| chr1 | 18808929 | 18808936 | 8 | 1:18808929-18808936 | 1.64E−40 | 6.63E−38 | 36.7727472 | 90.0271183 | −53.2543711 |
| chr1 | 18808938 | 18808941 | 4 | 1:18808938-18808941 | 1.64E−40 | 6.63E−38 | 36.7727472 | 90.0271183 | −53.2543711 |
| chr1 | 18808967 | 18808971 | 5 | 1:18808967-18808971 | 1.64E−40 | 6.63E−38 | 36.7727472 | 90.0271183 | −53.2543711 |
| chr1 | 18808991 | 18808994 | 4 | 1:18808991-18808994 | 1.64E−40 | 6.63E−38 | 36.7727472 | 90.0271183 | −53.2543711 |
| chr1 | 18809009 | 18809012 | 4 | 1:18809009-18809012 | 1.64E−40 | 6.63E−38 | 36.7727472 | 90.0271183 | −53.2543711 |
| chr1 | 18809028 | 18809036 | 9 | 1:18809028-18809036 | 1.64E−40 | 6.63E−38 | 36.7727472 | 90.0271183 | −53.2543711 |
| chr1 | 18809041 | 18809044 | 4 | 1:18809041-18809044 | 1.64E−40 | 6.63E−38 | 36.7727472 | 90.0271183 | −53.2543711 |
| chr1 | 18809052 | 18809055 | 4 | 1:18809052-18809055 | 1.64E−40 | 6.63E−38 | 36.7727472 | 90.0271183 | −53.2543711 |
| chr1 | 18809058 | 18809061 | 4 | 1:18809058-18809061 | 1.64E−40 | 6.63E−38 | 36.7727472 | 90.0271183 | −53.2543711 |
| chr1 | 18809064 | 18809068 | 5 | 1:18809064-18809068 | 1.64E−40 | 6.63E−38 | 36.7727472 | 90.0271183 | −53.2543711 |
| chr1 | 18809102 | 18809106 | 5 | 1:18809102-18809106 | 1.64E−40 | 6.63E−38 | 36.7727472 | 90.0271183 | −53.2543711 |
| chr1 | 18809112 | 18809115 | 4 | 1:18809112-18809115 | 1.64E−40 | 6.63E−38 | 36.7727472 | 90.0271183 | −53.2543711 |
| chr1 | 18809126 | 18809129 | 4 | 1:18809126-18809129 | 1.64E−40 | 6.63E−38 | 36.7727472 | 90.0271183 | −53.2543711 |
| chr1 | 18809494 | 18809497 | 4 | 1:18809494-18809497 | 1.64E−40 | 6.63E−38 | 36.7727472 | 90.0271183 | −53.2543711 |
| chr1 | 20141125 | 20141128 | 4 | 1:20141125-20141128 | 1.5E−15 | 3.59E−14 | 32.2386973 | 80.1381529 | −47.8994555 |
| chr1 | 20141136 | 20141139 | 4 | 1:20141136-20141139 | 1.5E−15 | 3.59E−14 | 32.2386973 | 80.1381529 | −47.8994555 |
| chr1 | 20141322 | 20141325 | 4 | 1:20141322-20141325 | 1.5E−15 | 3.59E−14 | 32.2386973 | 80.1381529 | −47.8994555 |
| chr1 | 20141344 | 20141347 | 4 | 1:20141344-20141347 | 1.5E−15 | 3.59E−14 | 32.2386973 | 80.1381529 | −47.8994555 |
| chr1 | 20141404 | 20141407 | 4 | 1:20141404-20141407 | 1.5E−15 | 3.59E−14 | 32.2386973 | 80.1381529 | −47.8994555 |
| chr1 | 20141442 | 20141445 | 4 | 1:20141442-20141445 | 1.5E−15 | 3.59E−14 | 32.2386973 | 80.1381529 | −47.8994555 |
| chr1 | 20141462 | 20141465 | 4 | 1:20141462-20141465 | 1.5E−15 | 3.59E−14 | 32.2386973 | 80.1381529 | −47.8994555 |
| chr1 | 22195529 | 22195532 | 4 | 1:22195529-22195532 | 5.41E−06 | 2.09E−05 | 42.4725 | 83.6422932 | −41.1697932 |
| chr1 | 32252163 | 32252166 | 4 | 1:32252163-32252166 | 2.06E−05 | 6.77E−05 | 17.3888889 | 71.0847953 | −53.6959064 |
| chr1 | 32252229 | 32252232 | 4 | 1:32252229-32252232 | 2.06E−05 | 6.77E−05 | 17.3888889 | 71.0847953 | −53.6959064 |
| chr1 | 32252239 | 32252242 | 4 | 1:32252239-32252242 | 2.06E−05 | 6.77E−05 | 17.3888889 | 71.0847953 | −53.6959064 |
| chr1 | 32252264 | 32252268 | 5 | 1:32252264-32252268 | 2.06E−05 | 6.77E−05 | 17.3888889 | 71.0847953 | −53.6959064 |
| chr1 | 32252425 | 32252428 | 4 | 1:32252425-32252428 | 2.06E−05 | 6.77E−05 | 17.3888889 | 71.0847953 | −53.6959064 |
| chr1 | 45103348 | 45103351 | 4 | 1:45103348-45103351 | 2.49E−08 | 1.68E−07 | 41.7444444 | 85.0616855 | −43.317241 |
| chr1 | 45103390 | 45103393 | 4 | 1:45103390-45103393 | 2.49E−08 | 1.68E−07 | 41.7444444 | 85.0616855 | −43.317241 |
| chr1 | 45106491 | 45106494 | 4 | 1:45106491-45106494 | 2.49E−08 | 1.68E−07 | 41.7444444 | 85.0616855 | −43.317241 |
| chr1 | 156215343 | 156215346 | 4 | 1:156215343-156215346 | 7E−11 | 7.84E−10 | 6.65714286 | 47.3143275 | −40.6571846 |
| chr1 | 156215371 | 156215374 | 4 | 1:156215371-156215374 | 7E−11 | 7.84E−10 | 6.65714286 | 47.3143275 | −40.6571846 |
| chr1 | 156215401 | 156215404 | 4 | 1:156215401-156215404 | 7E−11 | 7.84E−10 | 6.65714286 | 47.3143275 | −40.6571846 |
| chr1 | 156215416 | 156215420 | 5 | 1:156215416-156215420 | 7E−11 | 7.84E−10 | 6.65714286 | 47.3143275 | −40.6571846 |
| chr1 | 156215430 | 156215433 | 4 | 1:156215430-156215433 | 7E−11 | 7.84E−10 | 6.65714286 | 47.3143275 | −40.6571846 |
| chr1 | 156215445 | 156215448 | 4 | 1:156215445-156215448 | 7E−11 | 7.84E−10 | 6.65714286 | 47.3143275 | −40.6571846 |
| chr1 | 156215454 | 156215457 | 4 | 1:156215454-156215457 | 7E−11 | 7.84E−10 | 6.65714286 | 47.3143275 | −40.6571846 |
| chr1 | 156215491 | 156215496 | 6 | 1:156215491-156215496 | 7E−11 | 7.84E−10 | 6.65714286 | 47.3143275 | −40.6571846 |
| chr1 | 156215506 | 156215511 | 6 | 1:156215506-156215511 | 7E−11 | 7.84E−10 | 6.65714286 | 47.3143275 | −40.6571846 |
| chr1 | 156215520 | 156215523 | 4 | 1:156215520-156215523 | 7E−11 | 7.84E−10 | 6.65714286 | 47.3143275 | −40.6571846 |
| chr1 | 156215525 | 156215528 | 4 | 1:156215525-156215528 | 7E−11 | 7.84E−10 | 6.65714286 | 47.3143275 | −40.6571846 |
| chr1 | 156215530 | 156215533 | 4 | 1:156215530-156215533 | 7E−11 | 7.84E−10 | 6.65714286 | 47.3143275 | −40.6571846 |
| chr1 | 156215563 | 156215566 | 4 | 1:156215563-156215566 | 7E−11 | 7.84E−10 | 6.65714286 | 47.3143275 | −40.6571846 |
| chr1 | 156215590 | 156215598 | 9 | 1:156215590-156215598 | 7E−11 | 7.84E−10 | 6.65714286 | 47.3143275 | −40.6571846 |
| chr1 | 156215601 | 156215604 | 4 | 1:156215601-156215604 | 7E−11 | 7.84E−10 | 6.65714286 | 47.3143275 | −40.6571846 |
| chr1 | 156215708 | 156215711 | 4 | 1:156215708-156215711 | 7E−11 | 7.84E−10 | 6.65714286 | 47.3143275 | −40.6571846 |
| chr1 | 156215720 | 156215723 | 4 | 1:156215720-156215723 | 7E−11 | 7.84E−10 | 6.65714286 | 47.3143275 | −40.6571846 |
| chr1 | 156215726 | 156215733 | 8 | 1:156215726-156215733 | 7E−11 | 7.84E−10 | 6.65714286 | 47.3143275 | −40.6571846 |
| chr1 | 156215737 | 156215740 | 4 | 1:156215737-156215740 | 7E−11 | 7.84E−10 | 6.65714286 | 47.3143275 | −40.6571846 |
| chr1 | 203259747 | 203259755 | 9 | 1:203259747-203259755 | 1.99E−05 | 6.71E−05 | 0.67991453 | 44.7894737 | −44.1095592 |
| chr1 | 203259757 | 203259761 | 5 | 1:203259757-203259761 | 1.99E−05 | 6.71E−05 | 0.67991453 | 44.7894737 | −44.1095592 |
| chr1 | 203259770 | 203259774 | 5 | 1:203259770-203259774 | 1.99E−05 | 6.71E−05 | 0.67991453 | 44.7894737 | −44.1095592 |
| chr1 | 203259836 | 203259839 | 4 | 1:203259836-203259839 | 1.99E−05 | 6.71E−05 | 0.67991453 | 44.7894737 | −44.1095592 |
| chr1 | 203259850 | 203259853 | 4 | 1:203259850-203259853 | 1.99E−05 | 6.71E−05 | 0.67991453 | 44.7894737 | −44.1095592 |
| chr1 | 203259921 | 203259924 | 4 | 1:203259921-203259924 | 1.99E−05 | 6.71E−05 | 0.67991453 | 44.7894737 | −44.1095592 |
| chr1 | 236311219 | 236311222 | 4 | 1:236311219-236311222 | 0.000285 | 0.000676 | 72.7111111 | 32.631579 | 40.07953216 |
| chr1 | 236311342 | 236311345 | 4 | 1:236311342-236311345 | 0.000285 | 0.000676 | 72.7111111 | 32.631579 | 40.07953216 |
| chr1 | 237129554 | 237129557 | 4 | 1:237129554-237129557 | 8.06E−11 | 8.94E−10 | 36.3496345 | 79.1940719 | −42.8444374 |
| chr1 | 237134163 | 237134166 | 4 | 1:237134163-237134166 | 8.06E−11 | 8.94E−10 | 36.3496345 | 79.1940719 | −42.8444374 |
| chr1 | 237167480 | 237167483 | 4 | 1:237167480-237167483 | 8.06E−11 | 8.94E−10 | 36.3496345 | 79.1940719 | −42.8444374 |
| chr1 | 237194084 | 237194087 | 4 | 1:237194084-237194087 | 8.06E−11 | 8.94E−10 | 36.3496345 | 79.1940719 | −42.8444374 |
| chr1 | 237194093 | 237194096 | 4 | 1:237194093-237194096 | 8.06E−11 | 8.94E−10 | 36.3496345 | 79.1940719 | −42.8444374 |
| chr1 | 237194098 | 237194101 | 4 | 1:237194098-237194101 | 8.06E−11 | 8.94E−10 | 36.3496345 | 79.1940719 | −42.8444374 |
| chr1 | 237195314 | 237195317 | 4 | 1:237195314-237195317 | 8.06E−11 | 8.94E−10 | 36.3496345 | 79.1940719 | −42.8444374 |
| chr1 | 237195411 | 237195414 | 4 | 1:237195411-237195414 | 8.06E−11 | 8.94E−10 | 36.3496345 | 79.1940719 | −42.8444374 |
| chr1 | 237212336 | 237212339 | 4 | 1:237212336-237212339 | 4.76E−33 | 9.29E−31 | 36.2774713 | 76.4897424 | −40.2122712 |
| chr1 | 237220609 | 237220612 | 4 | 1:237220609-237220612 | 4.76E−33 | 9.29E−31 | 36.2774713 | 76.4897424 | −40.2122712 |
| chr1 | 237220641 | 237220648 | 8 | 1:237220641-237220648 | 4.76E−33 | 9.29E−31 | 36.2774713 | 76.4897424 | −40.2122712 |
| chr1 | 237222098 | 237222101 | 4 | 1:237222098-237222101 | 4.76E−33 | 9.29E−31 | 36.2774713 | 76.4897424 | −40.2122712 |
| chr1 | 237222106 | 237222109 | 4 | 1:237222106-237222109 | 4.76E−33 | 9.29E−31 | 36.2774713 | 76.4897424 | −40.2122712 |
| chr1 | 237235121 | 237235124 | 4 | 1:237235121-237235124 | 4.76E−33 | 9.29E−31 | 36.2774713 | 76.4897424 | −40.2122712 |
| chr1 | 237235204 | 237235207 | 4 | 1:237235204-237235207 | 4.76E−33 | 9.29E−31 | 36.2774713 | 76.4897424 | −40.2122712 |
| chr1 | 237235522 | 237235525 | 4 | 1:237235522-237235525 | 4.76E−33 | 9.29E−31 | 36.2774713 | 76.4897424 | −40.2122712 |
| chr1 | 237235567 | 237235573 | 7 | 1:237235567-237235573 | 4.76E−33 | 9.29E−31 | 36.2774713 | 76.4897424 | −40.2122712 |
| chr1 | 237235607 | 237235610 | 4 | 1:237235607-237235610 | 4.76E−33 | 9.29E−31 | 36.2774713 | 76.4897424 | −40.2122712 |
| chr1 | 237256273 | 237256277 | 5 | 1:237256273-237256277 | 4.76E−33 | 9.29E−31 | 36.2774713 | 76.4897424 | −40.2122712 |
| chr1 | 237256316 | 237256319 | 4 | 1:237256316-237256319 | 4.76E−33 | 9.29E−31 | 36.2774713 | 76.4897424 | −40.2122712 |
| chr1 | 237265786 | 237265789 | 4 | 1:237265786-237265789 | 4.76E−33 | 9.29E−31 | 36.2774713 | 76.4897424 | −40.2122712 |
| chr1 | 237265877 | 237265880 | 4 | 1:237265877-237265880 | 4.76E−33 | 9.29E−31 | 36.2774713 | 76.4897424 | −40.2122712 |
| chr1 | 237267786 | 237267789 | 4 | 1:237267786-237267789 | 4.76E−33 | 9.29E−31 | 36.2774713 | 76.4897424 | −40.2122712 |
| chr1 | 237267812 | 237267815 | 4 | 1:237267812-237267815 | 4.76E−33 | 9.29E−31 | 36.2774713 | 76.4897424 | −40.2122712 |
| chr1 | 237267886 | 237267889 | 4 | 1:237267886-237267889 | 4.76E−33 | 9.29E−31 | 36.2774713 | 76.4897424 | −40.2122712 |
| chr1 | 237306208 | 237306211 | 4 | 1:237306208-237306211 | 4.76E−33 | 9.29E−31 | 36.2774713 | 76.4897424 | −40.2122712 |
| chr1 | 237306260 | 237306263 | 4 | 1:237306260-237306263 | 4.76E−33 | 9.29E−31 | 36.2774713 | 76.4897424 | −40.2122712 |
| chr1 | 237343254 | 237343257 | 4 | 1:237343254-237343257 | 4.76E−33 | 9.29E−31 | 36.2774713 | 76.4897424 | −40.2122712 |
| chr1 | 237359169 | 237359172 | 4 | 1:237359169-237359172 | 4.76E−33 | 9.29E−31 | 36.2774713 | 76.4897424 | −40.2122712 |
| chr1 | 237383690 | 237383693 | 4 | 1:237383690-237383693 | 4.76E−33 | 9.29E−31 | 36.2774713 | 76.4897424 | −40.2122712 |
| chr1 | 237392760 | 237392763 | 4 | 1:237392760-237392763 | 4.76E−33 | 9.29E−31 | 36.2774713 | 76.4897424 | −40.2122712 |
| chr1 | 237408076 | 237408079 | 4 | 1:237408076-237408079 | 4.76E−33 | 9.29E−31 | 36.2774713 | 76.4897424 | −40.2122712 |
| chr1 | 237408091 | 237408094 | 4 | 1:237408091-237408094 | 4.76E−33 | 9.29E−31 | 36.2774713 | 76.4897424 | −40.2122712 |
| chr1 | 237473057 | 237473061 | 5 | 1:237473057-237473061 | 4.76E−33 | 9.29E−31 | 36.2774713 | 76.4897424 | −40.2122712 |
| chr1 | 237473071 | 237473074 | 4 | 1:237473071-237473074 | 4.76E−33 | 9.29E−31 | 36.2774713 | 76.4897424 | −40.2122712 |
| chr1 | 237473091 | 237473094 | 4 | 1:237473091-237473094 | 4.76E−33 | 9.29E−31 | 36.2774713 | 76.4897424 | −40.2122712 |
| chr1 | 237473164 | 237473167 | 4 | 1:237473164-237473167 | 4.76E−33 | 9.29E−31 | 36.2774713 | 76.4897424 | −40.2122712 |
| chr1 | 237538162 | 237538165 | 4 | 1:237538162-237538165 | 4.76E−33 | 9.29E−31 | 36.2774713 | 76.4897424 | −40.2122712 |
| chr1 | 237584276 | 237584279 | 4 | 1:237584276-237584279 | 4.76E−33 | 9.29E−31 | 36.2774713 | 76.4897424 | −40.2122712 |
| chr1 | 237661252 | 237661255 | 4 | 1:237661252-237661255 | 4.76E−33 | 9.29E−31 | 36.2774713 | 76.4897424 | −40.2122712 |
| chr1 | 237661262 | 237661265 | 4 | 1:237661262-237661265 | 4.76E−33 | 9.29E−31 | 36.2774713 | 76.4897424 | −40.2122712 |
| chr1 | 237661322 | 237661325 | 4 | 1:237661322-237661325 | 4.76E−33 | 9.29E−31 | 36.2774713 | 76.4897424 | −40.2122712 |
| chr1 | 237693526 | 237693529 | 4 | 1:237693526-237693529 | 4.76E−33 | 9.29E−31 | 36.2774713 | 76.4897424 | −40.2122712 |
| chr1 | 237693534 | 237693537 | 4 | 1:237693534-237693537 | 4.76E−33 | 9.29E−31 | 36.2774713 | 76.4897424 | −40.2122712 |
| chr1 | 237699930 | 237699933 | 4 | 1:237699930-237699933 | 4.76E−33 | 9.29E−31 | 36.2774713 | 76.4897424 | −40.2122712 |
| chr1 | 237699938 | 237699941 | 4 | 1:237699938-237699941 | 4.76E−33 | 9.29E−31 | 36.2774713 | 76.4897424 | −40.2122712 |
| chr1 | 237699988 | 237699991 | 4 | 1:237699988-237699991 | 4.76E−33 | 9.29E−31 | 36.2774713 | 76.4897424 | −40.2122712 |
| chr1 | 237809365 | 237809368 | 4 | 1:237809365-237809368 | 4.76E−33 | 9.29E−31 | 36.2774713 | 76.4897424 | −40.2122712 |
| chr1 | 237815260 | 237815263 | 4 | 1:237815260-237815263 | 4.76E−33 | 9.29E−31 | 36.2774713 | 76.4897424 | −40.2122712 |
| chr1 | 241402493 | 241402496 | 4 | 1:241402493-241402496 | 3.52E−07 | 1.85E−06 | 79.8267974 | 37.8637771 | 41.9630203 |
| chr1 | 241402516 | 241402519 | 4 | 1:241402516-241402519 | 3.52E−07 | 1.85E−06 | 79.8267974 | 37.8637771 | 41.9630203 |
| chr2 | 9248023 | 9248026 | 4 | 2:9248023-9248026 | 7.77E−05 | 0.000217 | 39.7125 | 88.375 | −48.6625 |
| chr2 | 9248368 | 9248371 | 4 | 2:9248368-9248371 | 7.77E−05 | 0.000217 | 39.7125 | 88.375 | −48.6625 |
| chr2 | 9248435 | 9248438 | 4 | 2:9248435-9248438 | 7.77E−05 | 0.000217 | 39.7125 | 88.375 | −48.6625 |
| chr2 | 9248469 | 9248472 | 4 | 2:9248469-9248472 | 7.77E−05 | 0.000217 | 39.7125 | 88.375 | −48.6625 |
| chr2 | 25299679 | 25299682 | 4 | 2:25299679-25299682 | 0.000363 | 0.000822 | 35.3825 | 77.4900929 | −42.1075929 |
| chr2 | 25299815 | 25299818 | 4 | 2:25299815-25299818 | 0.000363 | 0.000822 | 35.3825 | 77.4900929 | −42.1075929 |
| chr2 | 25299928 | 25299931 | 4 | 2:25299928-25299931 | 0.000363 | 0.000822 | 35.3825 | 77.4900929 | −42.1075929 |
| chr2 | 43102420 | 43102423 | 4 | 2:43102420-43102423 | 1.91E−06 | 8.28E−06 | 79.5705416 | 38.5468082 | 41.02373335 |
| chr2 | 43107259 | 43107262 | 4 | 2:43107259-43107262 | 1.91E−06 | 8.28E−06 | 79.5705416 | 38.5468082 | 41.02373335 |
| chr2 | 43107400 | 43107403 | 4 | 2:43107400-43107403 | 1.91E−06 | 8.28E−06 | 79.5705416 | 38.5468082 | 41.02373335 |
| chr2 | 43107414 | 43107417 | 4 | 2:43107414-43107417 | 1.91E−06 | 8.28E−06 | 79.5705416 | 38.5468082 | 41.02373335 |
| chr2 | 43107438 | 43107441 | 4 | 2:43107438-43107441 | 1.91E−06 | 8.28E−06 | 79.5705416 | 38.5468082 | 41.02373335 |
| chr2 | 43107456 | 43107459 | 4 | 2:43107456-43107459 | 1.91E−06 | 8.28E−06 | 79.5705416 | 38.5468082 | 41.02373335 |
| chr2 | 43107465 | 43107469 | 5 | 2:43107465-43107469 | 1.91E−06 | 8.28E−06 | 79.5705416 | 38.5468082 | 41.02373335 |
| chr2 | 43107514 | 43107517 | 4 | 2:43107514-43107517 | 1.91E−06 | 8.28E−06 | 79.5705416 | 38.5468082 | 41.02373335 |
| chr2 | 43107532 | 43107535 | 4 | 2:43107532-43107535 | 1.91E−06 | 8.28E−06 | 79.5705416 | 38.5468082 | 41.02373335 |
| chr2 | 43107650 | 43107653 | 4 | 2:43107650-43107653 | 1.91E−06 | 8.28E−06 | 79.5705416 | 38.5468082 | 41.02373335 |
| chr2 | 45235684 | 45235687 | 4 | 2:45235684-45235687 | 3.7E−07 | 1.91E−06 | 18.9833333 | 67.2982456 | −48.3149123 |
| chr2 | 45235702 | 45235705 | 4 | 2:45235702-45235705 | 3.7E−07 | 1.91E−06 | 18.9833333 | 67.2982456 | −48.3149123 |
| chr2 | 45235710 | 45235713 | 4 | 2:45235710-45235713 | 3.7E−07 | 1.91E−06 | 18.9833333 | 67.2982456 | −48.3149123 |
| chr2 | 207972376 | 207972379 | 4 | 2:207972376-207972379 | 7.77E−05 | 0.000217 | 30.5746528 | 80.4016813 | −49.8270285 |
| chr2 | 207972408 | 207972412 | 5 | 2:207972408-207972412 | 7.77E−05 | 0.000217 | 30.5746528 | 80.4016813 | −49.8270285 |
| chr2 | 220509654 | 220509657 | 4 | 2:220509654-220509657 | 2.9E−11 | 3.51E−10 | 31.31 | 76.8979532 | −45.5879532 |
| chr2 | 220509693 | 220509696 | 4 | 2:220509693-220509696 | 2.9E−11 | 3.51E−10 | 31.31 | 76.8979532 | −45.5879532 |
| chr2 | 220509705 | 220509709 | 5 | 2:220509705-220509709 | 2.9E−11 | 3.51E−10 | 31.31 | 76.8979532 | −45.5879532 |
| chr2 | 220509776 | 220509779 | 4 | 2:220509776-220509779 | 2.9E−11 | 3.51E−10 | 31.31 | 76.8979532 | −45.5879532 |
| chr2 | 220509830 | 220509833 | 4 | 2:220509830-220509833 | 2.9E−11 | 3.51E−10 | 31.31 | 76.8979532 | −45.5879532 |
| chr2 | 241233035 | 241233038 | 4 | 2:241233035-241233038 | 4.33E−06 | 1.73E−05 | 21.3222222 | 71.8994602 | −50.577238 |
| chr2 | 241233048 | 241233051 | 4 | 2:241233048-241233051 | 4.33E−06 | 1.73E−05 | 21.3222222 | 71.8994602 | −50.577238 |
| chr2 | 241233055 | 241233059 | 5 | 2:241233055-241233059 | 4.33E−06 | 1.73E−05 | 21.3222222 | 71.8994602 | −50.577238 |
| chr2 | 241233084 | 241233088 | 5 | 2:241233084-241233088 | 4.33E−06 | 1.73E−05 | 21.3222222 | 71.8994602 | −50.577238 |
| chr2 | 241233090 | 241233094 | 5 | 2:241233090-241233094 | 4.33E−06 | 1.73E−05 | 21.3222222 | 71.8994602 | −50.577238 |
| chr2 | 241233169 | 241233172 | 4 | 2:241233169-241233172 | 4.33E−06 | 1.73E−05 | 21.3222222 | 71.8994602 | −50.577238 |
| chr2 | 241233214 | 241233217 | 4 | 2:241233214-241233217 | 4.33E−06 | 1.73E−05 | 21.3222222 | 71.8994602 | −50.577238 |
| chr3 | 111431516 | 111431520 | 5 | 3:111431516-111431520 | 0.000291 | 0.000676 | 41.125 | 90.2090422 | −49.0840422 |
| chr3 | 111431559 | 111431562 | 4 | 3:111431559-111431562 | 0.000291 | 0.000676 | 41.125 | 90.2090422 | −49.0840422 |
| chr4 | 3309964 | 3309967 | 4 | 4:3309964-3309967 | 1.61E−07 | 9.12E−07 | 29.6368827 | 84.1175324 | −54.4806497 |
| chr4 | 3310004 | 3310007 | 4 | 4:3310004-3310007 | 1.61E−07 | 9.12E−07 | 29.6368827 | 84.1175324 | −54.4806497 |
| chr4 | 3311787 | 3311790 | 4 | 4:3311787-3311790 | 1.61E−07 | 9.12E−07 | 29.6368827 | 84.1175324 | −54.4806497 |
| chr4 | 3311917 | 3311920 | 4 | 4:3311917-3311920 | 1.61E−07 | 9.12E−07 | 29.6368827 | 84.1175324 | −54.4806497 |
| chr4 | 7228254 | 7228257 | 4 | 4:7228254-7228257 | 2.06E−05 | 6.77E−05 | 25.5 | 80.2776918 | −54.7776918 |
| chr4 | 7228263 | 7228269 | 7 | 4:7228263-7228269 | 2.06E−05 | 6.77E−05 | 25.5 | 80.2776918 | −54.7776918 |
| chr4 | 7228304 | 7228308 | 5 | 4:7228304-7228308 | 2.06E−05 | 6.77E−05 | 25.5 | 80.2776918 | −54.7776918 |
| chr4 | 7228329 | 7228332 | 4 | 4:7228329-7228332 | 2.06E−05 | 6.77E−05 | 25.5 | 80.2776918 | −54.7776918 |
| chr4 | 7326173 | 7326179 | 7 | 4:7326173-7326179 | 0.000103 | 0.000276 | 31.5775 | 72.4918301 | −40.9143301 |
| chr4 | 7326188 | 7326191 | 4 | 4:7326188-7326191 | 0.000103 | 0.000276 | 31.5775 | 72.4918301 | −40.9143301 |
| chr4 | 7326270 | 7326273 | 4 | 4:7326270-7326273 | 0.000103 | 0.000276 | 31.5775 | 72.4918301 | −40.9143301 |
| chr4 | 7326284 | 7326287 | 4 | 4:7326284-7326287 | 0.000103 | 0.000276 | 31.5775 | 72.4918301 | −40.9143301 |
| chr4 | 155758143 | 155758146 | 4 | 4:155758143-155758146 | 7.68E−09 | 5.83E−08 | 34.4404233 | 75.4663808 | −41.0259575 |
| chr4 | 155763679 | 155763682 | 4 | 4:155763679-155763682 | 7.68E−09 | 5.83E−08 | 34.4404233 | 75.4663808 | −41.0259575 |
| chr4 | 155763688 | 155763691 | 4 | 4:155763688-155763691 | 7.68E−09 | 5.83E−08 | 34.4404233 | 75.4663808 | −41.0259575 |
| chr4 | 155763695 | 155763698 | 4 | 4:155763695-155763698 | 7.68E−09 | 5.83E−08 | 34.4404233 | 75.4663808 | −41.0259575 |
| chr4 | 155812513 | 155812516 | 4 | 4:155812513-155812516 | 7.68E−09 | 5.83E−08 | 34.4404233 | 75.4663808 | −41.0259575 |
| chr4 | 155912924 | 155912927 | 4 | 4:155912924-155912927 | 7.68E−09 | 5.83E−08 | 34.4404233 | 75.4663808 | −41.0259575 |
| chr4 | 155965520 | 155965524 | 5 | 4:155965520-155965524 | 7.68E−09 | 5.83E−08 | 34.4404233 | 75.4663808 | −41.0259575 |
| chr4 | 155965532 | 155965535 | 4 | 4:155965532-155965535 | 7.68E−09 | 5.83E−08 | 34.4404233 | 75.4663808 | −41.0259575 |
| chr4 | 155965538 | 155965541 | 4 | 4:155965538-155965541 | 7.68E−09 | 5.83E−08 | 34.4404233 | 75.4663808 | −41.0259575 |
| chr4 | 155965544 | 155965547 | 4 | 4:155965544-155965547 | 7.68E−09 | 5.83E−08 | 34.4404233 | 75.4663808 | −41.0259575 |
| chr4 | 155965553 | 155965556 | 4 | 4:155965553-155965556 | 7.68E−09 | 5.83E−08 | 34.4404233 | 75.4663808 | −41.0259575 |
| chr4 | 155992200 | 155992203 | 4 | 4:155992200-155992203 | 7.68E−09 | 5.83E−08 | 34.4404233 | 75.4663808 | −41.0259575 |
| chr4 | 156002533 | 156002536 | 4 | 4:156002533-156002536 | 7.68E−09 | 5.83E−08 | 34.4404233 | 75.4663808 | −41.0259575 |
| chr5 | 1003895 | 1003898 | 4 | 5:1003895-1003898 | 1.97E−11 | 2.44E−10 | 11.1075694 | 51.5506412 | −40.4430717 |
| chr5 | 1004045 | 1004048 | 4 | 5:1004045-1004048 | 1.97E−11 | 2.44E−10 | 11.1075694 | 51.5506412 | −40.4430717 |
| chr5 | 1004051 | 1004054 | 4 | 5:1004051-1004054 | 1.97E−11 | 2.44E−10 | 11.1075694 | 51.5506412 | −40.4430717 |
| chr5 | 1004064 | 1004067 | 4 | 5:1004064-1004067 | 1.97E−11 | 2.44E−10 | 11.1075694 | 51.5506412 | −40.4430717 |
| chr5 | 1004077 | 1004080 | 4 | 5:1004077-1004080 | 1.97E−11 | 2.44E−10 | 11.1075694 | 51.5506412 | −40.4430717 |
| chr5 | 1004089 | 1004094 | 6 | 5:1004089-1004094 | 1.97E−11 | 2.44E−10 | 11.1075694 | 51.5506412 | −40.4430717 |
| chr5 | 1004118 | 1004121 | 4 | 5:1004118-1004121 | 1.97E−11 | 2.44E−10 | 11.1075694 | 51.5506412 | −40.4430717 |
| chr5 | 1004128 | 1004131 | 4 | 5:1004128-1004131 | 1.97E−11 | 2.44E−10 | 11.1075694 | 51.5506412 | −40.4430717 |
| chr5 | 1004135 | 1004140 | 6 | 5:1004135-1004140 | 1.97E−11 | 2.44E−10 | 11.1075694 | 51.5506412 | −40.4430717 |
| chr5 | 1004148 | 1004152 | 5 | 5:1004148-1004152 | 1.97E−11 | 2.44E−10 | 11.1075694 | 51.5506412 | −40.4430717 |
| chr5 | 1004157 | 1004161 | 5 | 5:1004157-1004161 | 1.97E−11 | 2.44E−10 | 11.1075694 | 51.5506412 | −40.4430717 |
| chr5 | 1004184 | 1004187 | 4 | 5:1004184-1004187 | 1.97E−11 | 2.44E−10 | 11.1075694 | 51.5506412 | −40.4430717 |
| chr5 | 1004198 | 1004201 | 4 | 5:1004198-1004201 | 1.97E−11 | 2.44E−10 | 11.1075694 | 51.5506412 | −40.4430717 |
| chr5 | 1004234 | 1004237 | 4 | 5:1004234-1004237 | 1.97E−11 | 2.44E−10 | 11.1075694 | 51.5506412 | −40.4430717 |
| chr5 | 1004243 | 1004254 | 12 | 5:1004243-1004254 | 1.97E−11 | 2.44E−10 | 11.1075694 | 51.5506412 | −40.4430717 |
| chr5 | 1004259 | 1004262 | 4 | 5:1004259-1004262 | 1.97E−11 | 2.44E−10 | 11.1075694 | 51.5506412 | −40.4430717 |
| chr5 | 1004269 | 1004272 | 4 | 5:1004269-1004272 | 1.97E−11 | 2.44E−10 | 11.1075694 | 51.5506412 | −40.4430717 |
| chr5 | 1004288 | 1004291 | 4 | 5:1004288-1004291 | 1.97E−11 | 2.44E−10 | 11.1075694 | 51.5506412 | −40.4430717 |
| chr5 | 1004304 | 1004307 | 4 | 5:1004304-1004307 | 1.97E−11 | 2.44E−10 | 11.1075694 | 51.5506412 | −40.4430717 |
| chr5 | 1004351 | 1004354 | 4 | 5:1004351-1004354 | 1.97E−11 | 2.44E−10 | 11.1075694 | 51.5506412 | −40.4430717 |
| chr5 | 1004357 | 1004362 | 6 | 5:1004357-1004362 | 1.97E−11 | 2.44E−10 | 11.1075694 | 51.5506412 | −40.4430717 |
| chr5 | 1004365 | 1004368 | 4 | 5:1004365-1004368 | 1.97E−11 | 2.44E−10 | 11.1075694 | 51.5506412 | −40.4430717 |
| chr5 | 134827336 | 134827349 | 14 | 5:134827336-134827349 | 3.37E−08 | 2.21E−07 | 11.62 | 60.8643963 | −49.2443963 |
| chr5 | 134827353 | 134827356 | 4 | 5:134827353-134827356 | 3.37E−08 | 2.21E−07 | 11.62 | 60.8643963 | −49.2443963 |
| chr5 | 134827364 | 134827367 | 4 | 5:134827364-134827367 | 3.37E−08 | 2.21E−07 | 11.62 | 60.8643963 | −49.2443963 |
| chr5 | 134827378 | 134827381 | 4 | 5:134827378-134827381 | 3.37E−08 | 2.21E−07 | 11.62 | 60.8643963 | −49.2443963 |
| chr5 | 138441347 | 138441353 | 7 | 5:138441347-138441353 | 7.77E−05 | 0.000217 | 9.51006944 | 82.3405573 | −72.8304878 |
| chr5 | 138441377 | 138441382 | 6 | 5:138441377-138441382 | 7.77E−05 | 0.000217 | 9.51006944 | 82.3405573 | −72.8304878 |
| chr5 | 138441561 | 138441564 | 4 | 5:138441561-138441564 | 7.77E−05 | 0.000217 | 9.51006944 | 82.3405573 | −72.8304878 |
| chr5 | 179401802 | 179401805 | 4 | 5:179401802-179401805 | 4.06E−05 | 0.000124 | 36.179798 | 95.4641148 | −59.2843169 |
| chr5 | 179401833 | 179401836 | 4 | 5:179401833-179401836 | 4.06E−05 | 0.000124 | 36.179798 | 95.4641148 | −59.2843169 |
| chr5 | 179401843 | 179401846 | 4 | 5:179401843-179401846 | 4.06E−05 | 0.000124 | 36.179798 | 95.4641148 | −59.2843169 |
| chr5 | 179401853 | 179401856 | 4 | 5:179401853-179401856 | 4.06E−05 | 0.000124 | 36.179798 | 95.4641148 | −59.2843169 |
| chr5 | 179401872 | 179401875 | 4 | 5:179401872-179401875 | 4.06E−05 | 0.000124 | 36.179798 | 95.4641148 | −59.2843169 |
| chr5 | 179588495 | 179588499 | 5 | 5:179588495-179588499 | 7.23E−14 | 1.34E−12 | 21.7844444 | 64.9108474 | −43.1264029 |
| chr5 | 179588504 | 179588507 | 4 | 5:179588504-179588507 | 7.23E−14 | 1.34E−12 | 21.7844444 | 64.9108474 | −43.1264029 |
| chr5 | 179588512 | 179588515 | 4 | 5:179588512-179588515 | 7.23E−14 | 1.34E−12 | 21.7844444 | 64.9108474 | −43.1264029 |
| chr5 | 179588528 | 179588533 | 6 | 5:179588528-179588533 | 7.23E−14 | 1.34E−12 | 21.7844444 | 64.9108474 | −43.1264029 |
| chr5 | 179588550 | 179588553 | 4 | 5:179588550-179588553 | 7.23E−14 | 1.34E−12 | 21.7844444 | 64.9108474 | −43.1264029 |
| chr5 | 179588590 | 179588594 | 5 | 5:179588590-179588594 | 7.23E−14 | 1.34E−12 | 21.7844444 | 64.9108474 | −43.1264029 |
| chr5 | 179588604 | 179588610 | 7 | 5:179588604-179588610 | 7.23E−14 | 1.34E−12 | 21.7844444 | 64.9108474 | −43.1264029 |
| chr5 | 179588617 | 179588620 | 4 | 5:179588617-179588620 | 7.23E−14 | 1.34E−12 | 21.7844444 | 64.9108474 | −43.1264029 |
| chr5 | 179588625 | 179588628 | 4 | 5:179588625-179588628 | 7.23E−14 | 1.34E−12 | 21.7844444 | 64.9108474 | −43.1264029 |
| chr5 | 179588631 | 179588634 | 4 | 5:179588631-179588634 | 7.23E−14 | 1.34E−12 | 21.7844444 | 64.9108474 | −43.1264029 |
| chr5 | 179588672 | 179588675 | 4 | 5:179588672-179588675 | 7.23E−14 | 1.34E−12 | 21.7844444 | 64.9108474 | −43.1264029 |
| chr5 | 179588687 | 179588690 | 4 | 5:179588687-179588690 | 7.23E−14 | 1.34E−12 | 21.7844444 | 64.9108474 | −43.1264029 |
| chr5 | 179588704 | 179588709 | 6 | 5:179588704-179588709 | 7.23E−14 | 1.34E−12 | 21.7844444 | 64.9108474 | −43.1264029 |
| chr5 | 179588719 | 179588722 | 4 | 5:179588719-179588722 | 7.23E−14 | 1.34E−12 | 21.7844444 | 64.9108474 | −43.1264029 |
| chr5 | 179597660 | 179597663 | 4 | 5:179597660-179597663 | 7.23E−14 | 1.34E−12 | 21.7844444 | 64.9108474 | −43.1264029 |
| chr5 | 179597771 | 179597774 | 4 | 5:179597771-179597774 | 7.23E−14 | 1.34E−12 | 21.7844444 | 64.9108474 | −43.1264029 |
| chr5 | 179597812 | 179597815 | 4 | 5:179597812-179597815 | 7.23E−14 | 1.34E−12 | 21.7844444 | 64.9108474 | −43.1264029 |
| chr6 | 64151793 | 64151797 | 5 | 6:64151793-64151797 | 7.37E−05 | 0.000212 | 42.8307692 | 83.0698381 | −40.2390688 |
| chr6 | 157549787 | 157549790 | 4 | 6:157549787-157549790 | 0.000186 | 0.000469 | 40.0541667 | 84.4736842 | −44.4195175 |
| chr6 | 157549797 | 157549800 | 4 | 6:157549797-157549800 | 0.000186 | 0.000469 | 40.0541667 | 84.4736842 | −44.4195175 |
| chr6 | 157549848 | 157549851 | 4 | 6:157549848-157549851 | 0.000186 | 0.000469 | 40.0541667 | 84.4736842 | −44.4195175 |
| chr6 | 157550494 | 157550497 | 4 | 6:157550494-157550497 | 0.000186 | 0.000469 | 40.0541667 | 84.4736842 | −44.4195175 |
| chr6 | 157550509 | 157550513 | 5 | 6:157550509-157550513 | 0.000186 | 0.000469 | 40.0541667 | 84.4736842 | −44.4195175 |
| chr7 | 1447696 | 1447699 | 4 | 7:1447696-1447699 | 2.06E−05 | 6.77E−05 | 9.97530864 | 67.7017544 | −57.7264457 |
| chr7 | 1447734 | 1447738 | 5 | 7:1447734-1447738 | 2.06E−05 | 6.77E−05 | 9.97530864 | 67.7017544 | −57.7264457 |
| chr7 | 1447746 | 1447750 | 5 | 7:1447746-1447750 | 2.06E−05 | 6.77E−05 | 9.97530864 | 67.7017544 | −57.7264457 |
| chr7 | 1447760 | 1447763 | 4 | 7:1447760-1447763 | 2.06E−05 | 6.77E−05 | 9.97530864 | 67.7017544 | −57.7264457 |
| chr7 | 1447774 | 1447779 | 6 | 7:1447774-1447779 | 2.06E−05 | 6.77E−05 | 9.97530864 | 67.7017544 | −57.7264457 |
| chr7 | 1447782 | 1447785 | 4 | 7:1447782-1447785 | 2.06E−05 | 6.77E−05 | 9.97530864 | 67.7017544 | −57.7264457 |
| chr7 | 2006576 | 2006579 | 4 | 7:2006576-2006579 | 6.45E−09 | 4.97E−08 | 38.9466667 | 88.9785575 | −50.0318908 |
| chr7 | 2006674 | 2006678 | 5 | 7:2006674-2006678 | 6.45E−09 | 4.97E−08 | 38.9466667 | 88.9785575 | −50.0318908 |
| chr7 | 2007262 | 2007265 | 4 | 7:2007262-2007265 | 6.45E−09 | 4.97E−08 | 38.9466667 | 88.9785575 | −50.0318908 |
| chr7 | 2007279 | 2007282 | 4 | 7:2007279-2007282 | 6.45E−09 | 4.97E−08 | 38.9466667 | 88.9785575 | −50.0318908 |
| chr7 | 2007309 | 2007312 | 4 | 7:2007309-2007312 | 6.45E−09 | 4.97E−08 | 38.9466667 | 88.9785575 | −50.0318908 |
| chr7 | 2007394 | 2007397 | 4 | 7:2007394-2007397 | 6.45E−09 | 4.97E−08 | 38.9466667 | 88.9785575 | −50.0318908 |
| chr7 | 2968280 | 2968283 | 4 | 7:2968280-2968283 | 3.6E−15 | 8.16E−14 | 16.374729 | 83.1922882 | −66.8175592 |
| chr7 | 2968288 | 2968295 | 8 | 7:2968288-2968295 | 3.6E−15 | 8.16E−14 | 16.374729 | 83.1922882 | −66.8175592 |
| chr7 | 2968314 | 2968318 | 5 | 7:2968314-2968318 | 3.6E−15 | 8.16E−14 | 16.374729 | 83.1922882 | −66.8175592 |
| chr7 | 2968320 | 2968324 | 5 | 7:2968320-2968324 | 3.6E−15 | 8.16E−14 | 16.374729 | 83.1922882 | −66.8175592 |
| chr7 | 2968352 | 2968355 | 4 | 7:2968352-2968355 | 3.6E−15 | 8.16E−14 | 16.374729 | 83.1922882 | −66.8175592 |
| chr7 | 2968357 | 2968360 | 4 | 7:2968357-2968360 | 3.6E−15 | 8.16E−14 | 16.374729 | 83.1922882 | −66.8175592 |
| chr7 | 2968405 | 2968408 | 4 | 7:2968405-2968408 | 3.6E−15 | 8.16E−14 | 16.374729 | 83.1922882 | −66.8175592 |
| chr7 | 2968411 | 2968414 | 4 | 7:2968411-2968414 | 3.6E−15 | 8.16E−14 | 16.374729 | 83.1922882 | −66.8175592 |
| chr7 | 2968426 | 2968429 | 4 | 7:2968426-2968429 | 3.6E−15 | 8.16E−14 | 16.374729 | 83.1922882 | −66.8175592 |
| chr7 | 2968440 | 2968448 | 9 | 7:2968440-2968448 | 3.6E−15 | 8.16E−14 | 16.374729 | 83.1922882 | −66.8175592 |
| chr7 | 2968454 | 2968461 | 8 | 7:2968454-2968461 | 3.6E−15 | 8.16E−14 | 16.374729 | 83.1922882 | −66.8175592 |
| chr7 | 2968489 | 2968501 | 13 | 7:2968489-2968501 | 3.6E−15 | 8.16E−14 | 16.374729 | 83.1922882 | −66.8175592 |
| chr7 | 2968504 | 2968510 | 7 | 7:2968504-2968510 | 3.6E−15 | 8.16E−14 | 16.374729 | 83.1922882 | −66.8175592 |
| chr7 | 2968528 | 2968532 | 5 | 7:2968528-2968532 | 3.6E−15 | 8.16E−14 | 16.374729 | 83.1922882 | −66.8175592 |
| chr7 | 2968552 | 2968555 | 4 | 7:2968552-2968555 | 3.6E−15 | 8.16E−14 | 16.374729 | 83.1922882 | −66.8175592 |
| chr7 | 2968558 | 2968561 | 4 | 7:2968558-2968561 | 3.6E−15 | 8.16E−14 | 16.374729 | 83.1922882 | −66.8175592 |
| chr7 | 2968565 | 2968571 | 7 | 7:2968565-2968571 | 3.6E−15 | 8.16E−14 | 16.374729 | 83.1922882 | −66.8175592 |
| chr7 | 2968585 | 2968589 | 5 | 7:2968585-2968589 | 3.6E−15 | 8.16E−14 | 16.374729 | 83.1922882 | −66.8175592 |
| chr7 | 2968593 | 2968597 | 5 | 7:2968593-2968597 | 3.6E−15 | 8.16E−14 | 16.374729 | 83.1922882 | −66.8175592 |
| chr7 | 2968625 | 2968629 | 5 | 7:2968625-2968629 | 3.6E−15 | 8.16E−14 | 16.374729 | 83.1922882 | −66.8175592 |
| chr7 | 2968684 | 2968687 | 4 | 7:2968684-2968687 | 3.6E−15 | 8.16E−14 | 16.374729 | 83.1922882 | −66.8175592 |
| chr7 | 2968706 | 2968709 | 4 | 7:2968706-2968709 | 3.6E−15 | 8.16E−14 | 16.374729 | 83.1922882 | −66.8175592 |
| chr7 | 26667584 | 26667587 | 4 | 7:26667584-26667587 | 4.44E−06 | 1.77E−05 | 31.3314815 | 78.7902612 | −47.4587798 |
| chr7 | 26667613 | 26667616 | 4 | 7:26667613-26667616 | 4.44E−06 | 1.77E−05 | 31.3314815 | 78.7902612 | −47.4587798 |
| chr7 | 26667674 | 26667677 | 4 | 7:26667674-26667677 | 4.44E−06 | 1.77E−05 | 31.3314815 | 78.7902612 | −47.4587798 |
| chr7 | 26667709 | 26667712 | 4 | 7:26667709-26667712 | 4.44E−06 | 1.77E−05 | 31.3314815 | 78.7902612 | −47.4587798 |
| chr7 | 35505816 | 35505819 | 4 | 7:35505816-35505819 | 5.41E−06 | 2.09E−05 | 32.21 | 73.0269737 | −40.8169737 |
| chr7 | 35505825 | 35505829 | 5 | 7:35505825-35505829 | 5.41E−06 | 2.09E−05 | 32.21 | 73.0269737 | −40.8169737 |
| chr7 | 41086116 | 41086120 | 5 | 7:41086116-41086120 | 1.42E−06 | 6.37E−06 | 38.1656566 | 80.9018435 | −42.736187 |
| chr7 | 41086123 | 41086127 | 5 | 7:41086123-41086127 | 1.42E−06 | 6.37E−06 | 38.1656566 | 80.9018435 | −42.736187 |
| chr7 | 41086140 | 41086143 | 4 | 7:41086140-41086143 | 1.42E−06 | 6.37E−06 | 38.1656566 | 80.9018435 | −42.736187 |
| chr7 | 101622017 | 101622021 | 5 | 7:101622017-101622021 | 1.42E−06 | 6.37E−06 | 35.0141414 | 89.8965976 | −54.8824561 |
| chr7 | 101622057 | 101622061 | 5 | 7:101622057-101622061 | 1.42E−06 | 6.37E−06 | 35.0141414 | 89.8965976 | −54.8824561 |
| chr7 | 101622176 | 101622186 | 11 | 7:101622176-101622186 | 1.42E−06 | 6.37E−06 | 35.0141414 | 89.8965976 | −54.8824561 |
| chr7 | 115992730 | 115992733 | 4 | 7:115992730-115992733 | 2.38E−05 | 7.72E−05 | 32.7184524 | 78.2632772 | −45.5448249 |
| chr7 | 115992814 | 115992817 | 4 | 7:115992814-115992817 | 2.38E−05 | 7.72E−05 | 32.7184524 | 78.2632772 | −45.5448249 |
| chr7 | 115998982 | 115998985 | 4 | 7:115998982-115998985 | 2.38E−05 | 7.72E−05 | 32.7184524 | 78.2632772 | −45.5448249 |
| chr7 | 115999026 | 115999029 | 4 | 7:115999026-115999029 | 2.38E−05 | 7.72E−05 | 32.7184524 | 78.2632772 | −45.5448249 |
| chr7 | 115999448 | 115999451 | 4 | 7:115999448-115999451 | 2.38E−05 | 7.72E−05 | 32.7184524 | 78.2632772 | −45.5448249 |
| chr7 | 115999464 | 115999467 | 4 | 7:115999464-115999467 | 2.38E−05 | 7.72E−05 | 32.7184524 | 78.2632772 | −45.5448249 |
| chr7 | 116099337 | 116099340 | 4 | 7:116099337-116099340 | 2.38E−05 | 7.72E−05 | 32.7184524 | 78.2632772 | −45.5448249 |
| chr7 | 150668588 | 150668591 | 4 | 7:150668588-150668591 | 2.98E−07 | 1.6E−06 | 26.8697368 | 70.0041694 | −43.1344325 |
| chr7 | 150669120 | 150669123 | 4 | 7:150669120-150669123 | 2.98E−07 | 1.6E−06 | 26.8697368 | 70.0041694 | −43.1344325 |
| chr7 | 150811991 | 150811994 | 4 | 7:150811991-150811994 | 7.43E−57 | 1.01E−53 | 58.645298 | 14.201114 | 44.44418402 |
| chr7 | 150812075 | 150812078 | 4 | 7:150812075-150812078 | 7.43E−57 | 1.01E−53 | 58.645298 | 14.201114 | 44.44418402 |
| chr7 | 150812111 | 150812114 | 4 | 7:150812111-150812114 | 7.43E−57 | 1.01E−53 | 58.645298 | 14.201114 | 44.44418402 |
| chr7 | 150812201 | 150812204 | 4 | 7:150812201-150812204 | 7.43E−57 | 1.01E−53 | 58.645298 | 14.201114 | 44.44418402 |
| chr7 | 150812215 | 150812220 | 6 | 7:150812215-150812220 | 7.43E−57 | 1.01E−53 | 58.645298 | 14.201114 | 44.44418402 |
| chr7 | 150812398 | 150812401 | 4 | 7:150812398-150812401 | 7.43E−57 | 1.01E−53 | 58.645298 | 14.201114 | 44.44418402 |
| chr7 | 150812660 | 150812665 | 6 | 7:150812660-150812665 | 7.43E−57 | 1.01E−53 | 58.645298 | 14.201114 | 44.44418402 |
| chr7 | 157423275 | 157423278 | 4 | 7:157423275-157423278 | 0.000291 | 0.000676 | 79.9428571 | 26.6679198 | 53.27493734 |
| chr7 | 157623246 | 157623252 | 7 | 7:157623246-157623252 | 5.41E−06 | 2.09E−05 | 21.73 | 72.3932749 | −50.6632749 |
| chr8 | 46929775 | 46929778 | 4 | 8:46929775-46929778 | 1.67E−06 | 7.38E−06 | 33.4936508 | 78.9231903 | −45.4295395 |
| chr8 | 46935836 | 46935839 | 4 | 8:46935836-46935839 | 1.67E−06 | 7.38E−06 | 33.4936508 | 78.9231903 | −45.4295395 |
| chr8 | 46938035 | 46938038 | 4 | 8:46938035-46938038 | 1.67E−06 | 7.38E−06 | 33.4936508 | 78.9231903 | −45.4295395 |
| chr8 | 46938044 | 46938047 | 4 | 8:46938044-46938047 | 1.67E−06 | 7.38E−06 | 33.4936508 | 78.9231903 | −45.4295395 |
| chr8 | 46938657 | 46938660 | 4 | 8:46938657-46938660 | 1.67E−06 | 7.38E−06 | 33.4936508 | 78.9231903 | −45.4295395 |
| chr8 | 46938693 | 46938696 | 4 | 8:46938693-46938696 | 1.67E−06 | 7.38E−06 | 33.4936508 | 78.9231903 | −45.4295395 |
| chr8 | 46938702 | 46938705 | 4 | 8:46938702-46938705 | 1.67E−06 | 7.38E−06 | 33.4936508 | 78.9231903 | −45.4295395 |
| chr8 | 46940361 | 46940364 | 4 | 8:46940361-46940364 | 1.67E−06 | 7.38E−06 | 33.4936508 | 78.9231903 | −45.4295395 |
| chr8 | 46940406 | 46940409 | 4 | 8:46940406-46940409 | 1.67E−06 | 7.38E−06 | 33.4936508 | 78.9231903 | −45.4295395 |
| chr8 | 58062363 | 58062366 | 4 | 8:58062363-58062366 | 1.12E−22 | 6.97E−21 | 35.125965 | 75.9882834 | −40.8623184 |
| chr8 | 58062426 | 58062429 | 4 | 8:58062426-58062429 | 1.12E−22 | 6.97E−21 | 35.125965 | 75.9882834 | −40.8623184 |
| chr8 | 58062463 | 58062466 | 4 | 8:58062463-58062466 | 1.12E−22 | 6.97E−21 | 35.125965 | 75.9882834 | −40.8623184 |
| chr8 | 58096948 | 58096951 | 4 | 8:58096948-58096951 | 1.12E−22 | 6.97E−21 | 35.125965 | 75.9882834 | −40.8623184 |
| chr8 | 58115010 | 58115014 | 5 | 8:58115010-58115014 | 1.12E−22 | 6.97E−21 | 35.125965 | 75.9882834 | −40.8623184 |
| chr8 | 58115060 | 58115063 | 4 | 8:58115060-58115063 | 1.12E−22 | 6.97E−21 | 35.125965 | 75.9882834 | −40.8623184 |
| chr8 | 58115154 | 58115157 | 4 | 8:58115154-58115157 | 1.12E−22 | 6.97E−21 | 35.125965 | 75.9882834 | −40.8623184 |
| chr8 | 58115225 | 58115228 | 4 | 8:58115225-58115228 | 1.12E−22 | 6.97E−21 | 35.125965 | 75.9882834 | −40.8623184 |
| chr8 | 58115343 | 58115346 | 4 | 8:58115343-58115346 | 1.12E−22 | 6.97E−21 | 35.125965 | 75.9882834 | −40.8623184 |
| chr8 | 58115362 | 58115365 | 4 | 8:58115362-58115365 | 1.12E−22 | 6.97E−21 | 35.125965 | 75.9882834 | −40.8623184 |
| chr8 | 58122819 | 58122822 | 4 | 8:58122819-58122822 | 1.12E−22 | 6.97E−21 | 35.125965 | 75.9882834 | −40.8623184 |
| chr8 | 58122836 | 58122839 | 4 | 8:58122836-58122839 | 1.12E−22 | 6.97E−21 | 35.125965 | 75.9882834 | −40.8623184 |
| chr8 | 58122848 | 58122851 | 4 | 8:58122848-58122851 | 1.12E−22 | 6.97E−21 | 35.125965 | 75.9882834 | −40.8623184 |
| chr8 | 58122861 | 58122864 | 4 | 8:58122861-58122864 | 1.12E−22 | 6.97E−21 | 35.125965 | 75.9882834 | −40.8623184 |
| chr8 | 58123008 | 58123011 | 4 | 8:58123008-58123011 | 1.12E−22 | 6.97E−21 | 35.125965 | 75.9882834 | −40.8623184 |
| chr8 | 58126620 | 58126623 | 4 | 8:58126620-58126623 | 1.12E−22 | 6.97E−21 | 35.125965 | 75.9882834 | −40.8623184 |
| chr8 | 58126666 | 58126669 | 4 | 8:58126666-58126669 | 1.12E−22 | 6.97E−21 | 35.125965 | 75.9882834 | −40.8623184 |
| chr8 | 58126874 | 58126877 | 4 | 8:58126874-58126877 | 1.12E−22 | 6.97E−21 | 35.125965 | 75.9882834 | −40.8623184 |
| chr8 | 58127096 | 58127099 | 4 | 8:58127096-58127099 | 1.12E−22 | 6.97E−21 | 35.125965 | 75.9882834 | −40.8623184 |
| chr8 | 58127515 | 58127518 | 4 | 8:58127515-58127518 | 1.12E−22 | 6.97E−21 | 35.125965 | 75.9882834 | −40.8623184 |
| chr8 | 58127556 | 58127559 | 4 | 8:58127556-58127559 | 1.12E−22 | 6.97E−21 | 35.125965 | 75.9882834 | −40.8623184 |
| chr8 | 58127613 | 58127616 | 4 | 8:58127613-58127616 | 1.12E−22 | 6.97E−21 | 35.125965 | 75.9882834 | −40.8623184 |
| chr8 | 58130004 | 58130007 | 4 | 8:58130004-58130007 | 1.12E−22 | 6.97E−21 | 35.125965 | 75.9882834 | −40.8623184 |
| chr8 | 58130021 | 58130024 | 4 | 8 58130021-58130024 | 1.12E−22 | 6.97E−21 | 35.125965 | 75.9882834 | −40.8623184 |
| chr8 | 58130039 | 58130042 | 4 | 8:58130039-58130042 | 1.12E−22 | 6.97E−21 | 35.125965 | 75.9882834 | −40.8623184 |
| chr8 | 58131654 | 58131657 | 4 | 8:58131654-58131657 | 1.12E−22 | 6.97E−21 | 35.125965 | 75.9882834 | −40.8623184 |
| chr8 | 58131665 | 58131668 | 4 | 8:58131665-58131668 | 1.12E−22 | 6.97E−21 | 35.125965 | 75.9882834 | −40.8623184 |
| chr8 | 58131710 | 58131713 | 4 | 8:58131710-58131713 | 1.12E−22 | 6.97E−21 | 35.125965 | 75.9882834 | −40.8623184 |
| chr8 | 58131715 | 58131718 | 4 | 8:58131715-58131718 | 1.12E−22 | 6.97E−21 | 35.125965 | 75.9882834 | −40.8623184 |
| chr8 | 58131843 | 58131846 | 4 | 8:58131843-58131846 | 1.12E−22 | 6.97E−21 | 35.125965 | 75.9882834 | −40.8623184 |
| chr8 | 58131870 | 58131873 | 4 | 8:58131870-58131873 | 1.12E−22 | 6.97E−21 | 35.125965 | 75.9882834 | −40.8623184 |
| chr8 | 58133184 | 58133187 | 4 | 8:58133184-58133187 | 1.12E−22 | 6.97E−21 | 35.125965 | 75.9882834 | −40.8623184 |
| chr8 | 58133215 | 58133218 | 4 | 8:58133215-58133218 | 1.12E−22 | 6.97E−21 | 35.125965 | 75.9882834 | −40.8623184 |
| chr8 | 58133315 | 58133318 | 4 | 8:58133315-58133318 | 1.12E−22 | 6.97E−21 | 35.125965 | 75.9882834 | −40.8623184 |
| chr8 | 58133743 | 58133746 | 4 | 8:58133743-58133746 | 1.12E−22 | 6.97E−21 | 35.125965 | 75.9882834 | −40.8623184 |
| chr8 | 58171196 | 58171199 | 4 | 8:58171196-58171199 | 1.12E−22 | 6.97E−21 | 35.125965 | 75.9882834 | −40.8623184 |
| chr8 | 58171236 | 58171243 | 8 | 8:58171236-58171243 | 1.12E−22 | 6.97E−21 | 35.125965 | 75.9882834 | −40.8623184 |
| chr8 | 58192020 | 58192023 | 4 | 8:58192020-58192023 | 1.12E−22 | 6.97E−21 | 35.125965 | 75.9882834 | −40.8623184 |
| chr8 | 61777862 | 61777865 | 4 | 8:61777862-61777865 | 1.69E−06 | 7.42E−06 | 30.0859259 | 81.0350877 | −50.9491618 |
| chr8 | 61821531 | 61821534 | 4 | 8:61821531-61821534 | 1.69E−06 | 7.42E−06 | 30.0859259 | 81.0350877 | −50.9491618 |
| chr8 | 61821546 | 61821549 | 4 | 8:61821546-61821549 | 1.69E−06 | 7.42E−06 | 30.0859259 | 81.0350877 | −50.9491618 |
| chr8 | 61821578 | 61821581 | 4 | 8:61821578-61821581 | 1.69E−06 | 7.42E−06 | 30.0859259 | 81.0350877 | −50.9491618 |
| chr8 | 73471538 | 73471541 | 4 | 8:73471538-73471541 | 1.1E−12 | 1.69E−11 | 80.9555556 | 37.9347368 | 43.02081871 |
| chr8 | 73480053 | 73480056 | 4 | 8:73480053-73480056 | 1.1E−12 | 1.69E−11 | 80.9555556 | 37.9347368 | 43.02081871 |
| chr8 | 73551842 | 73551845 | 4 | 8:73551842-73551845 | 1.1E−12 | 1.69E−11 | 80.9555556 | 37.9347368 | 43.02081871 |
| chr8 | 73849013 | 73849016 | 4 | 8:73849013-73849016 | 1.1E−12 | 1.69E−11 | 80.9555556 | 37.9347368 | 43.02081871 |
| chr8 | 73849032 | 73849035 | 4 | 8:73849032-73849035 | 1.1E−12 | 1.69E−11 | 80.9555556 | 37.9347368 | 43.02081871 |
| chr8 | 73849038 | 73849041 | 4 | 8:73849038-73849041 | 1.1E−12 | 1.69E−11 | 80.9555556 | 37.9347368 | 43.02081871 |
| chr8 | 73849051 | 73849055 | 5 | 8:73849051-73849055 | 1.1E−12 | 1.69E−11 | 80.9555556 | 37.9347368 | 43.02081871 |
| chr8 | 73849060 | 73849063 | 4 | 8:73849060-73849063 | 1.1E−12 | 1.69E−11 | 80.9555556 | 37.9347368 | 43.02081871 |
| chr8 | 104117894 | 104117897 | 4 | 8:104117894-104117897 | 0.000291 | 0.000676 | 31.6142857 | 77.0601504 | −45.4458647 |
| chr8 | 140929837 | 140929840 | 4 | 8:140929837-140929840 | 3.16E−07 | 1.68E−06 | 41.7329365 | 82.9832916 | −41.2503551 |
| chr8 | 140930939 | 140930942 | 4 | 8:140930939-140930942 | 3.16E−07 | 1.68E−06 | 41.7329365 | 82.9832916 | −41.2503551 |
| chr8 | 140931316 | 140931319 | 4 | 8:140931316-140931319 | 3.16E−07 | 1.68E−06 | 41.7329365 | 82.9832916 | −41.2503551 |
| chr8 | 142367903 | 142367906 | 4 | 8:142367903-142367906 | 3.72E−06 | 1.51E−05 | 76.5785714 | 30.8796993 | 45.69887218 |
| chr8 | 142367951 | 142367955 | 5 | 8:142367951-142367955 | 3.72E−06 | 1.51E−05 | 76.5785714 | 30.8796993 | 45.69887218 |
| chr8 | 142367989 | 142367992 | 4 | 8:142367989-142367992 | 3.72E−06 | 1.51E−05 | 76.5785714 | 30.8796993 | 45.69887218 |
| chr9 | 117049844 | 117049847 | 4 | 9:117049844-117049847 | 7.77E−05 | 0.000217 | 29.1 | 70.1389319 | −41.0389319 |
| chr9 | 117049850 | 117049853 | 4 | 9:117049850-117049853 | 7.77E−05 | 0.000217 | 29.1 | 70.1389319 | −41.0389319 |
| chr9 | 117049862 | 117049865 | 4 | 9:117049862-117049865 | 7.77E−05 | 0.000217 | 29.1 | 70.1389319 | −41.0389319 |
| chr9 | 117049885 | 117049888 | 4 | 9:117049885-117049888 | 7.77E−05 | 0.000217 | 29.1 | 70.1389319 | −41.0389319 |
| chr9 | 127105722 | 127105725 | 4 | 9:127105722-127105725 | 6.44E−06 | 2.44E−05 | 30.5935897 | 73.1520877 | −42.5584979 |
| chr9 | 127105763 | 127105766 | 4 | 9:127105763-127105766 | 6.44E−06 | 2.44E−05 | 30.5935897 | 73.1520877 | −42.5584979 |
| chr9 | 129895923 | 129895926 | 4 | 9:129895923-129895926 | 2.06E−05 | 6.77E−05 | 20.3950617 | 72.9491338 | −52.5540721 |
| chr9 | 136007375 | 136007378 | 4 | 9:136007375-136007378 | 1.87E−05 | 6.33E−05 | 25.1384615 | 72.007859 | −46.8693975 |
| chr9 | 136007484 | 136007488 | 5 | 9:136007484-136007488 | 1.87E−05 | 6.33E−05 | 25.1384615 | 72.007859 | −46.8693975 |
| chr9 | 136007594 | 136007597 | 4 | 9:136007594-136007597 | 1.87E−05 | 6.33E−05 | 25.1384615 | 72.007859 | −46.8693975 |
| chr9 | 136007599 | 136007602 | 4 | 9:136007599-136007602 | 1.87E−05 | 6.33E−05 | 25.1384615 | 72.007859 | −46.8693975 |
| chr9 | 136007615 | 136007618 | 4 | 9:136007615-136007618 | 1.87E−05 | 6.33E−05 | 25.1384615 | 72.007859 | −46.8693975 |
| chr9 | 136007712 | 136007715 | 4 | 9:136007712-136007715 | 1.87E−05 | 6.33E−05 | 25.1384615 | 72.007859 | −46.8693975 |
| chr9 | 136324027 | 136324030 | 4 | 9:136324027-136324030 | 9.08E−05 | 0.000247 | 32.17 | 74.6578947 | −42.4878947 |
| chr9 | 136324037 | 136324040 | 4 | 9:136324037-136324040 | 9.08E−05 | 0.000247 | 32.17 | 74.6578947 | −42.4878947 |
| chr9 | 136324064 | 136324067 | 4 | 9:136324064-136324067 | 9.08E−05 | 0.000247 | 32.17 | 74.6578947 | −42.4878947 |
| chr9 | 138688868 | 138688871 | 4 | 9:138688868-138688871 | 1.3E−05 | 4.59E−05 | 13.1574786 | 54.1659919 | −41.0085133 |
| chr9 | 138688876 | 138688879 | 4 | 9:138688876-138688879 | 1.3E−05 | 4.59E−05 | 13.1574786 | 54.1659919 | −41.0085133 |
| chr9 | 138688902 | 138688906 | 5 | 9:138688902-138688906 | 1.3E−05 | 4.59E−05 | 13.1574786 | 54.1659919 | −41.0085133 |
| chr9 | 138688936 | 138688939 | 4 | 9:138688936-138688939 | 1.3E−05 | 4.59E−05 | 13.1574786 | 54.1659919 | −41.0085133 |
| chr9 | 138689094 | 138689100 | 7 | 9:138689094-138689100 | 1.3E−05 | 4.59E−05 | 13.1574786 | 54.1659919 | −41.0085133 |
| chr9 | 138689107 | 138689110 | 4 | 9:138689107-138689110 | 1.3E−05 | 4.59E−05 | 13.1574786 | 54.1659919 | −41.0085133 |
| chr9 | 138689483 | 138689486 | 4 | 9:138689483-138689486 | 1.3E−05 | 4.59E−05 | 13.1574786 | 54.1659919 | −41.0085133 |
| chr9 | 138719312 | 138719315 | 4 | 9:138719312-138719315 | 0.000291 | 0.000676 | 85.6142857 | 40.3609023 | 45.25338346 |
| chr9 | 138719386 | 138719389 | 4 | 9:138719386-138719389 | 0.000291 | 0.000676 | 85.6142857 | 40.3609023 | 45.25338346 |
| chr9 | 139419669 | 139419672 | 4 | 9:139419669-139419672 | 2.04E−16 | 5.52E−15 | 28.5274425 | 76.1990621 | −47.6716196 |
| chr9 | 139419769 | 139419772 | 4 | 9:139419769-139419772 | 2.04E−16 | 5.52E−15 | 28.5274425 | 76.1990621 | −47.6716196 |
| chr9 | 139419814 | 139419817 | 4 | 9:139419814-139419817 | 2.04E−16 | 5.52E−15 | 28.5274425 | 76.1990621 | −47.6716196 |
| chr9 | 139419865 | 139419875 | 11 | 9:139419865-139419875 | 2.04E−16 | 5.52E−15 | 28.5274425 | 76.1990621 | −47.6716196 |
| chr9 | 139419924 | 139419927 | 4 | 9:139419924-139419927 | 2.04E−16 | 5.52E−15 | 28.5274425 | 76.1990621 | −47.6716196 |
| chr9 | 139419933 | 139419941 | 9 | 9:139419933-139419941 | 2.04E−16 | 5.52E−15 | 28.5274425 | 76.1990621 | −47.6716196 |
| chr9 | 139419956 | 139419959 | 4 | 9:139419956-139419959 | 2.04E−16 | 5.52E−15 | 28.5274425 | 76.1990621 | −47.6716196 |
| chr9 | 139420550 | 139420553 | 4 | 9:139420550-139420553 | 2.04E−16 | 5.52E−15 | 28.5274425 | 76.1990621 | −47.6716196 |
| chr9 | 139420561 | 139420564 | 4 | 9:139420561-139420564 | 2.04E−16 | 5.52E−15 | 28.5274425 | 76.1990621 | −47.6716196 |
| chr9 | 139420578 | 139420581 | 4 | 9:139420578-139420581 | 2.04E−16 | 5.52E−15 | 28.5274425 | 76.1990621 | −47.6716196 |
| chr9 | 139420602 | 139420605 | 4 | 9:139420602-139420605 | 2.04E−16 | 5.52E−15 | 28.5274425 | 76.1990621 | −47.6716196 |
| chr9 | 139420641 | 139420644 | 4 | 9:139420641-139420644 | 2.04E−16 | 5.52E−15 | 28.5274425 | 76.1990621 | −47.6716196 |
| chr9 | 139420862 | 139420865 | 4 | 9:139420862-139420865 | 2.04E−16 | 5.52E−15 | 28.5274425 | 76.1990621 | −47.6716196 |
| chr9 | 139420914 | 139420917 | 4 | 9:139420914-139420917 | 2.04E−16 | 5.52E−15 | 28.5274425 | 76.1990621 | −47.6716196 |
| chr9 | 139421144 | 139421150 | 7 | 9:139421144-139421150 | 2.04E−16 | 5.52E−15 | 28.5274425 | 76.1990621 | −47.6716196 |
| chr9 | 139421220 | 139421223 | 4 | 9:139421220-139421223 | 2.04E−16 | 5.52E−15 | 28.5274425 | 76.1990621 | −47.6716196 |
| chr9 | 139421241 | 139421244 | 4 | 9:139421241-139421244 | 2.04E−16 | 5.52E−15 | 28.5274425 | 76.1990621 | −47.6716196 |
| chr9 | 139421257 | 139421263 | 7 | 9:139421257-139421263 | 2.04E−16 | 5.52E−15 | 28.5274425 | 76.1990621 | −47.6716196 |
| chr9 | 139421267 | 139421270 | 4 | 9:139421267-139421270 | 2.04E−16 | 5.52E−15 | 28.5274425 | 76.1990621 | −47.6716196 |
| chr9 | 139421321 | 139421324 | 4 | 9:139421321-139421324 | 2.04E−16 | 5.52E−15 | 28.5274425 | 76.1990621 | −47.6716196 |
| chr9 | 139421342 | 139421345 | 4 | 9:139421342-139421345 | 2.04E−16 | 5.52E−15 | 28.5274425 | 76.1990621 | −47.6716196 |
| chr9 | 139421362 | 139421365 | 4 | 9:139421362-139421365 | 2.04E−16 | 5.52E−15 | 28.5274425 | 76.1990621 | −47.6716196 |
| chr9 | 139422178 | 139422181 | 4 | 9:139422178-139422181 | 2.04E−16 | 5.52E−15 | 28.5274425 | 76.1990621 | −47.6716196 |
| chr9 | 139422203 | 139422206 | 4 | 9:139422203-139422206 | 2.04E−16 | 5.52E−15 | 28.5274425 | 76.1990621 | −47.6716196 |
| chr9 | 139422218 | 139422224 | 7 | 9:139422218-139422224 | 2.04E−16 | 5.52E−15 | 28.5274425 | 76.1990621 | −47.6716196 |
| chr9 | 139422266 | 139422273 | 8 | 9:139422266-139422273 | 2.04E−16 | 5.52E−15 | 28.5274425 | 76.1990621 | −47.6716196 |
| chr9 | 139422290 | 139422293 | 4 | 9:139422290-139422293 | 2.04E−16 | 5.52E−15 | 28.5274425 | 76.1990621 | −47.6716196 |
| chr9 | 139422314 | 139422317 | 4 | 9:139422314-139422317 | 2.04E−16 | 5.52E−15 | 28.5274425 | 76.1990621 | −47.6716196 |
| chr10 | 23976532 | 23976535 | 4 | 10:23976532-23976535 | 4.11E−05 | 0.000125 | 42.7975309 | 83.8691927 | −41.0716618 |
| chr10 | 23976581 | 23976585 | 5 | 10:23976581-23976585 | 4.11E−05 | 0.000125 | 42.7975309 | 83.8691927 | −41.0716618 |
| chr10 | 72489844 | 72489847 | 4 | 10:72489844-72489847 | 7.77E−05 | 0.000217 | 14.8125 | 83.7697368 | −68.9572368 |
| chr10 | 72489870 | 72489879 | 10 | 10:72489870-72489879 | 7.77E−05 | 0.000217 | 14.8125 | 83.7697368 | −68.9572368 |
| chr10 | 72489889 | 72489892 | 4 | 10:72489889-72489892 | 7.77E−05 | 0.000217 | 14.8125 | 83.7697368 | −68.9572368 |
| chr10 | 72489949 | 72489952 | 4 | 10:72489949-72489952 | 7.77E−05 | 0.000217 | 14.8125 | 83.7697368 | −68.9572368 |
| chr10 | 72489967 | 72489970 | 4 | 10:72489967-72489970 | 7.77E−05 | 0.000217 | 14.8125 | 83.7697368 | −68.9572368 |
| chr10 | 72489983 | 72489986 | 4 | 10:72489983-72489986 | 7.77E−05 | 0.000217 | 14.8125 | 83.7697368 | −68.9572368 |
| chr10 | 119590052 | 119590055 | 4 | 10:119590052-119590055 | 0.000291 | 0.000676 | 38.1928571 | 84.8942946 | −46.7014374 |
| chr10 | 134562610 | 134562613 | 4 | 10:134562610-134562613 | 1.84E−12 | 2.71E−11 | 47.6996732 | 92.0681115 | −44.3684383 |
| chr10 | 134563052 | 134563055 | 4 | 10:134563052-134563055 | 1.84E−12 | 2.71E−11 | 47.6996732 | 92.0681115 | −44.3684383 |
| chr10 | 134563060 | 134563064 | 5 | 10:134563060-134563064 | 1.84E−12 | 2.71E−11 | 47.6996732 | 92.0681115 | −44.3684383 |
| chr10 | 134563098 | 134563102 | 5 | 10:134563098-134563102 | 1.84E−12 | 2.71E−11 | 47.6996732 | 92.0681115 | −44.3684383 |
| chr10 | 134563109 | 134563112 | 4 | 10:134563109-134563112 | 1.84E−12 | 2.71E−11 | 47.6996732 | 92.0681115 | −44.3684383 |
| chr10 | 134563144 | 134563147 | 4 | 10:134563144-134563147 | 1.84E−12 | 2.71E−11 | 47.6996732 | 92.0681115 | −44.3684383 |
| chr10 | 134563149 | 134563152 | 4 | 10:134563149-134563152 | 1.84E−12 | 2.71E−11 | 47.6996732 | 92.0681115 | −44.3684383 |
| chr10 | 134563186 | 134563189 | 4 | 10:134563186-134563189 | 1.84E−12 | 2.71E−11 | 47.6996732 | 92.0681115 | −44.3684383 |
| chr10 | 134563199 | 134563202 | 4 | 10:134563199-134563202 | 1.84E−12 | 2.71E−11 | 47.6996732 | 92.0681115 | −44.3684383 |
| chr11 | 2495501 | 2495504 | 4 | 11:2495501-2495504 | 1.08E−05 | 3.88E−05 | 45.36 | 86.8263158 | −41.4663158 |
| chr11 | 2495543 | 2495546 | 4 | 11:2495543-2495546 | 1.08E−05 | 3.88E−05 | 45.36 | 86.8263158 | −41.4663158 |
| chr11 | 2495585 | 2495588 | 4 | 11:2495585-2495588 | 1.08E−05 | 3.88E−05 | 45.36 | 86.8263158 | −41.4663158 |
| chr11 | 2495609 | 2495612 | 4 | 11:2495609-2495612 | 1.08E−05 | 3.88E−05 | 45.36 | 86.8263158 | −41.4663158 |
| chr11 | 68967954 | 68967957 | 4 | 11:68967954-68967957 | 4.06E−11 | 4.78E−10 | 17.6117205 | 59.9311859 | −42.3194653 |
| chr11 | 68967994 | 68967997 | 4 | 11:68967994-68967997 | 4.06E−11 | 4.78E−10 | 17.6117205 | 59.9311859 | −42.3194653 |
| chr11 | 68968016 | 68968019 | 4 | 11:68968016-68968019 | 4.06E−11 | 4.78E−10 | 17.6117205 | 59.9311859 | −42.3194653 |
| chr11 | 68968032 | 68968035 | 4 | 11:68968032-68968035 | 4.06E−11 | 4.78E−10 | 17.6117205 | 59.9311859 | −42.3194653 |
| chr11 | 68968042 | 68968045 | 4 | 11:68968042-68968045 | 4.06E−11 | 4.78E−10 | 17.6117205 | 59.9311859 | −42.3194653 |
| chr11 | 68968141 | 68968148 | 8 | 11:68968141-68968148 | 4.06E−11 | 4.78E−10 | 17.6117205 | 59.9311859 | −42.3194653 |
| chr11 | 68968151 | 68968154 | 4 | 11:68968151-68968154 | 4.06E−11 | 4.78E−10 | 17.6117205 | 59.9311859 | −42.3194653 |
| chr11 | 68968156 | 68968159 | 4 | 11:68968156-68968159 | 4.06E−11 | 4.78E−10 | 17.6117205 | 59.9311859 | −42.3194653 |
| chr11 | 68968182 | 68968185 | 4 | 11:68968182-68968185 | 4.06E−11 | 4.78E−10 | 17.6117205 | 59.9311859 | −42.3194653 |
| chr11 | 68968214 | 68968217 | 4 | 11:68968214-68968217 | 4.06E−11 | 4.78E−10 | 17.6117205 | 59.9311859 | −42.3194653 |
| chr11 | 68968240 | 68968243 | 4 | 11:68968240-68968243 | 4.06E−11 | 4.78E−10 | 17.6117205 | 59.9311859 | −42.3194653 |
| chr11 | 68968267 | 68968272 | 6 | 11:68968267-68968272 | 4.06E−11 | 4.78E−10 | 17.6117205 | 59.9311859 | −42.3194653 |
| chr11 | 70851529 | 70851532 | 4 | 11:70851529-70851532 | 1.93E−05 | 6.51E−05 | 21.759375 | 68.6953306 | −46.9359556 |
| chr11 | 70851574 | 70851578 | 5 | 11:70851574-70851578 | 1.93E−05 | 6.51E−05 | 21.759375 | 68.6953306 | −46.9359556 |
| chr11 | 70851603 | 70851607 | 5 | 11:70851603-70851607 | 1.93E−05 | 6.51E−05 | 21.759375 | 68.6953306 | −46.9359556 |
| chr11 | 70851619 | 70851622 | 4 | 11:70851619-70851622 | 1.93E−05 | 6.51E−05 | 21.759375 | 68.6953306 | −46.9359556 |
| chr11 | 70851638 | 70851641 | 4 | 11:70851638-70851641 | 1.93E−05 | 6.51E−05 | 21.759375 | 68.6953306 | −46.9359556 |
| chr11 | 70851713 | 70851717 | 5 | 11:70851713-70851717 | 1.93E−05 | 6.51E−05 | 21.759375 | 68.6953306 | −46.9359556 |
| chr11 | 70851722 | 70851725 | 4 | 11:70851722-70851725 | 1.93E−05 | 6.51E−05 | 21.759375 | 68.6953306 | −46.9359556 |
| chr11 | 70851730 | 70851733 | 4 | 11:70851730-70851733 | 1.93E−05 | 6.51E−05 | 21.759375 | 68.6953306 | −46.9359556 |
| chr11 | 70851827 | 70851830 | 4 | 11:70851827-70851830 | 1.93E−05 | 6.51E−05 | 21.759375 | 68.6953306 | −46.9359556 |
| chr11 | 70851836 | 70851839 | 4 | 11:70851836-70851839 | 1.93E−05 | 6.51E−05 | 21.759375 | 68.6953306 | −46.9359556 |
| chr11 | 70851859 | 70851862 | 4 | 11:70851859-70851862 | 1.93E−05 | 6.51E−05 | 21.759375 | 68.6953306 | −46.9359556 |
| chr11 | 70851868 | 70851872 | 5 | 11:70851868-70851872 | 1.93E−05 | 6.51E−05 | 21.759375 | 68.6953306 | −46.9359556 |
| chr11 | 70851883 | 70851886 | 4 | 11:70851883-70851886 | 1.93E−05 | 6.51E−05 | 21.759375 | 68.6953306 | −46.9359556 |
| chr11 | 70851889 | 70851892 | 4 | 11:70851889-70851892 | 1.93E−05 | 6.51E−05 | 21.759375 | 68.6953306 | −46.9359556 |
| chr11 | 70851914 | 70851917 | 4 | 11:70851914-70851917 | 1.93E−05 | 6.51E−05 | 21.759375 | 68.6953306 | −46.9359556 |
| chr11 | 70862301 | 70862304 | 4 | 11:70862301-70862304 | 7.77E−05 | 0.000217 | 43.3479167 | 94.027595 | −50.6796784 |
| chr11 | 70862379 | 70862382 | 4 | 11:70862379-70862382 | 7.77E−05 | 0.000217 | 43.3479167 | 94.027595 | −50.6796784 |
| chr11 | 70862397 | 70862400 | 4 | 11:70862397-70862400 | 7.77E−05 | 0.000217 | 43.3479167 | 94.027595 | −50.6796784 |
| chr11 | 70862405 | 70862408 | 4 | 11:70862405-70862408 | 7.77E−05 | 0.000217 | 43.3479167 | 94.027595 | −50.6796784 |
| chr11 | 111265961 | 111265964 | 4 | 11:111265961-111265964 | 0.000291 | 0.000676 | 6.06825397 | 50.5948204 | −44.5265664 |
| chr11 | 111266000 | 111266004 | 5 | 11:111266000-111266004 | 0.000291 | 0.000676 | 6.06825397 | 50.5948204 | −44.5265664 |
| chr11 | 111266014 | 111266017 | 4 | 11:111266014-111266017 | 0.000291 | 0.000676 | 6.06825397 | 50.5948204 | −44.5265664 |
| chr11 | 111266031 | 111266034 | 4 | 11:111266031-111266034 | 0.000291 | 0.000676 | 6.06825397 | 50.5948204 | −44.5265664 |
| chr11 | 111266119 | 111266122 | 4 | 11:111266119-111266122 | 0.000291 | 0.000676 | 6.06825397 | 50.5948204 | −44.5265664 |
| chr11 | 115829517 | 115829520 | 4 | 11:115829517-115829520 | 5.81E−10 | 5.5E−09 | 37.9495727 | 84.3771508 | −46.4275782 |
| chr11 | 115829550 | 115829553 | 4 | 11:115829550-115829553 | 5.81E−10 | 5.5E−09 | 37.9495727 | 84.3771508 | −46.4275782 |
| chr11 | 115830020 | 115830023 | 4 | 11:115830020-115830023 | 5.81E−10 | 5.5E−09 | 37.9495727 | 84.3771508 | −46.4275782 |
| chr11 | 115830200 | 115830203 | 4 | 11:115830200-115830203 | 5.81E−10 | 5.5E−09 | 37.9495727 | 84.3771508 | −46.4275782 |
| chr11 | 133902554 | 133902557 | 4 | 11:133902554-133902557 | 2.19E−07 | 1.21E−06 | 14.5684211 | 56.6813861 | −42.1129651 |
| chr11 | 133902596 | 133902600 | 5 | 11:133902596-133902600 | 2.19E−07 | 1.21E−06 | 14.5684211 | 56.6813861 | −42.1129651 |
| chr11 | 133902615 | 133902618 | 4 | 11:133902615-133902618 | 2.19E−07 | 1.21E−06 | 14.5684211 | 56.6813861 | −42.1129651 |
| chr11 | 133902626 | 133902629 | 4 | 11:133902626-133902629 | 2.19E−07 | 1.21E−06 | 14.5684211 | 56.6813861 | −42.1129651 |
| chr11 | 133902656 | 133902659 | 4 | 11:133902656-133902659 | 2.19E−07 | 1.21E−06 | 14.5684211 | 56.6813861 | −42.1129651 |
| chr11 | 133902669 | 133902672 | 4 | 11:133902669-133902672 | 2.19E−07 | 1.21E−06 | 14.5684211 | 56.6813861 | −42.1129651 |
| chr11 | 133902689 | 133902692 | 4 | 11:133902689-133902692 | 2.19E−07 | 1.21E−06 | 14.5684211 | 56.6813861 | −42.1129651 |
| chr11 | 133902724 | 133902731 | 8 | 11:133902724-133902731 | 2.19E−07 | 1.21E−06 | 14.5684211 | 56.6813861 | −42.1129651 |
| chr11 | 133902737 | 133902740 | 4 | 11:133902737-133902740 | 2.19E−07 | 1.21E−06 | 14.5684211 | 56.6813861 | −42.1129651 |
| chr11 | 133902744 | 133902747 | 4 | 11:133902744-133902747 | 2.19E−07 | 1.21E−06 | 14.5684211 | 56.6813861 | −42.1129651 |
| chr11 | 133902766 | 133902769 | 4 | 11:133902766-133902769 | 2.19E−07 | 1.21E−06 | 14.5684211 | 56.6813861 | −42.1129651 |
| chr12 | 1957456 | 1957459 | 4 | 12:1957456-1957459 | 0.000291 | 0.000676 | 18.0571429 | 60.7448032 | −42.6876603 |
| chr12 | 1957466 | 1957469 | 4 | 12:1957466-1957469 | 0.000291 | 0.000676 | 18.0571429 | 60.7448032 | −42.6876603 |
| chr12 | 1957486 | 1957490 | 5 | 12:1957486-1957490 | 0.000291 | 0.000676 | 18.0571429 | 60.7448032 | −42.6876603 |
| chr12 | 1957501 | 1957504 | 4 | 12:1957501-1957504 | 0.000291 | 0.000676 | 18.0571429 | 60.7448032 | −42.6876603 |
| chr12 | 1957566 | 1957569 | 4 | 12:1957566-1957569 | 0.000291 | 0.000676 | 18.0571429 | 60.7448032 | −42.6876603 |
| chr12 | 34488345 | 34488348 | 4 | 12:34488345-34488348 | 7.77E−05 | 0.000217 | 83.8666667 | 43.7828947 | 40.08377193 |
| chr12 | 34488352 | 34488356 | 5 | 12:34488352-34488356 | 7.77E−05 | 0.000217 | 83.8666667 | 43.7828947 | 40.08377193 |
| chr12 | 66111480 | 66111483 | 4 | 12:66111480-66111483 | 7.77E−05 | 0.000217 | 38.5 | 84.0003655 | −45.5003655 |
| chr12 | 66111548 | 66111551 | 4 | 12:66111548-66111551 | 7.77E−05 | 0.000217 | 38.5 | 84.0003655 | −45.5003655 |
| chr12 | 66114657 | 66114661 | 5 | 12:66114657-66114661 | 7.77E−05 | 0.000217 | 38.5 | 84.0003655 | −45.5003655 |
| chr12 | 66114687 | 66114690 | 4 | 12:66114687-66114690 | 7.77E−05 | 0.000217 | 38.5 | 84.0003655 | −45.5003655 |
| chr12 | 66114695 | 66114698 | 4 | 12:66114695-66114698 | 7.77E−05 | 0.000217 | 38.5 | 84.0003655 | −45.5003655 |
| chr12 | 66262212 | 66262215 | 4 | 12:66262212-66262215 | 0.000291 | 0.000676 | 19.9115646 | 73.0637163 | −53.1521517 |
| chr12 | 66262228 | 66262236 | 9 | 12:66262228-66262236 | 0.000291 | 0.000676 | 19.9115646 | 73.0637163 | −53.1521517 |
| chr12 | 115109512 | 115109515 | 4 | 12:115109512-115109515 | 5.38E−32 | 9.46E−30 | 64.2223214 | 14.3712575 | 49.85106389 |
| chr12 | 115109529 | 115109532 | 4 | 12:115109529-115109532 | 5.38E−32 | 9.46E−30 | 64.2223214 | 14.3712575 | 49.85106389 |
| chr12 | 115109718 | 115109721 | 4 | 12:115109718-115109721 | 5.38E−32 | 9.46E−30 | 64.2223214 | 14.3712575 | 49.85106389 |
| chr12 | 115109960 | 115109963 | 4 | 12:115109960-115109963 | 5.38E−32 | 9.46E−30 | 64.2223214 | 14.3712575 | 49.85106389 |
| chr12 | 115112024 | 115112027 | 4 | 12:115112024-115112027 | 5.38E−32 | 9.46E−30 | 64.2223214 | 14.3712575 | 49.85106389 |
| chr12 | 115112037 | 115112041 | 5 | 12:115112037-115112041 | 5.38E−32 | 9.46E−30 | 64.2223214 | 14.3712575 | 49.85106389 |
| chr12 | 124947867 | 124947870 | 4 | 12:124947867-124947870 | 1.69E−06 | 7.42E−06 | 39.4483333 | 91.1611076 | −51.7127743 |
| chr12 | 124948139 | 124948142 | 4 | 12:124948139-124948142 | 1.69E−06 | 7.42E−06 | 39.4483333 | 91.1611076 | −51.7127743 |
| chr13 | 114523664 | 114523667 | 4 | 13:114523664-114523667 | 2.72E−20 | 1.23E−18 | 13.0425746 | 55.3672276 | −42.3246531 |
| chr13 | 114523669 | 114523673 | 5 | 13:114523669-114523673 | 2.72E−20 | 1.23E−18 | 13.0425746 | 55.3672276 | −42.3246531 |
| chr13 | 114523717 | 114523721 | 5 | 13:114523717-114523721 | 2.72E−20 | 1.23E−18 | 13.0425746 | 55.3672276 | −42.3246531 |
| chr13 | 114523760 | 114523764 | 5 | 13:114523760-114523764 | 2.72E−20 | 1.23E−18 | 13.0425746 | 55.3672276 | −42.3246531 |
| chr13 | 114523787 | 114523791 | 5 | 13:114523787-114523791 | 2.72E−20 | 1.23E−18 | 13.0425746 | 55.3672276 | −42.3246531 |
| chr13 | 114523796 | 114523799 | 4 | 13:114523796-114523799 | 2.72E−20 | 1.23E−18 | 13.0425746 | 55.3672276 | −42.3246531 |
| chr13 | 114523819 | 114523824 | 6 | 13:114523819-114523824 | 2.72E−20 | 1.23E−18 | 13.0425746 | 55.3672276 | −42.3246531 |
| chr13 | 114523826 | 114523829 | 4 | 13:114523826-114523829 | 2.72E−20 | 1.23E−18 | 13.0425746 | 55.3672276 | −42.3246531 |
| chr13 | 114523875 | 114523878 | 4 | 13:114523875-114523878 | 2.72E−20 | 1.23E−18 | 13.0425746 | 55.3672276 | −42.3246531 |
| chr13 | 114523883 | 114523886 | 4 | 13:114523883-114523886 | 2.72E−20 | 1.23E−18 | 13.0425746 | 55.3672276 | −42.3246531 |
| chr13 | 114523896 | 114523902 | 7 | 13:114523896-114523902 | 2.72E−20 | 1.23E−18 | 13.0425746 | 55.3672276 | −42.3246531 |
| chr13 | 114523904 | 114523907 | 4 | 13:114523904-114523907 | 2.72E−20 | 1.23E−18 | 13.0425746 | 55.3672276 | −42.3246531 |
| chr13 | 114523923 | 114523927 | 5 | 13:114523923-114523927 | 2.72E−20 | 1.23E−18 | 13.0425746 | 55.3672276 | −42.3246531 |
| chr13 | 114523948 | 114523954 | 7 | 13:114523948-114523954 | 2.72E−20 | 1.23E−18 | 13.0425746 | 55.3672276 | −42.3246531 |
| chr13 | 114523958 | 114523964 | 7 | 13:114523958-114523964 | 2.72E−20 | 1.23E−18 | 13.0425746 | 55.3672276 | −42.3246531 |
| chr13 | 114523970 | 114523974 | 5 | 13:114523970-114523974 | 2.72E−20 | 1.23E−18 | 13.0425746 | 55.3672276 | −42.3246531 |
| chr13 | 114523982 | 114523985 | 4 | 13:114523982-114523985 | 2.72E−20 | 1.23E−18 | 13.0425746 | 55.3672276 | −42.3246531 |
| chr13 | 114523996 | 114524000 | 5 | 13:114523996-114524000 | 2.72E−20 | 1.23E−18 | 13.0425746 | 55.3672276 | −42.3246531 |
| chr13 | 114524029 | 114524032 | 4 | 13:114524029-114524032 | 2.72E−20 | 1.23E−18 | 13.0425746 | 55.3672276 | −42.3246531 |
| chr13 | 114524093 | 114524096 | 4 | 13:114524093-114524096 | 2.72E−20 | 1.23E−18 | 13.0425746 | 55.3672276 | −42.3246531 |
| chr13 | 114524130 | 114524133 | 4 | 13:114524130-114524133 | 2.72E−20 | 1.23E−18 | 13.0425746 | 55.3672276 | −42.3246531 |
| chr13 | 114524137 | 114524144 | 8 | 13:114524137-114524144 | 2.72E−20 | 1.23E−18 | 13.0425746 | 55.3672276 | −42.3246531 |
| chr13 | 114524149 | 114524152 | 4 | 13:114524149-114524152 | 2.72E−20 | 1.23E−18 | 13.0425746 | 55.3672276 | −42.3246531 |
| chr13 | 114524157 | 114524160 | 4 | 13:114524157-114524160 | 2.72E−20 | 1.23E−18 | 13.0425746 | 55.3672276 | −42.3246531 |
| chr13 | 114524420 | 114524423 | 4 | 13:114524420-114524423 | 2.72E−20 | 1.23E−18 | 13.0425746 | 55.3672276 | −42.3246531 |
| chr13 | 114524429 | 114524432 | 4 | 13:114524429-114524432 | 2.72E−20 | 1.23E−18 | 13.0425746 | 55.3672276 | −42.3246531 |
| chr13 | 114524439 | 114524442 | 4 | 13:114524439-114524442 | 2.72E−20 | 1.23E−18 | 13.0425746 | 55.3672276 | −42.3246531 |
| chr13 | 114524473 | 114524477 | 5 | 13:114524473-114524477 | 2.72E−20 | 1.23E−18 | 13.0425746 | 55.3672276 | −42.3246531 |
| chr13 | 114524509 | 114524512 | 4 | 13:114524509-114524512 | 2.72E−20 | 1.23E−18 | 13.0425746 | 55.3672276 | −42.3246531 |
| chr13 | 114524517 | 114524520 | 4 | 13:114524517-114524520 | 2.72E−20 | 1.23E−18 | 13.0425746 | 55.3672276 | −42.3246531 |
| chr13 | 114524583 | 114524588 | 6 | 13:114524583-114524588 | 2.72E−20 | I.23E−18 | 13.0425746 | 55.3672276 | −42.3246531 |
| chr14 | 20684518 | 20684521 | 4 | 14:20684518-20684521 | 9.92E−06 | 3.59E−05 | 32.7 | 75.0816388 | −42.3816388 |
| chr14 | 20684528 | 20684531 | 4 | 14:20684528-20684531 | 9.92E−06 | 3.59E−05 | 32.7 | 75.0816388 | −42.3816388 |
| chr14 | 76957115 | 76957119 | 5 | 14:76957115-76957119 | 7.77E−05 | 0.000217 | 26.340625 | 77.3523392 | −51.0117142 |
| chr14 | 76957138 | 76957141 | 4 | 14:76957138-76957141 | 7.77E−05 | 0.000217 | 26.340625 | 77.3523392 | −51.0117142 |
| chr14 | 76957152 | 76957156 | 5 | 14:76957152-76957156 | 7.77E−05 | 0.000217 | 26.340625 | 77.3523392 | −51.0117142 |
| chr14 | 76957199 | 76957202 | 4 | 14:76957199-76957202 | 7.77E−05 | 0.000217 | 26.340625 | 77.3523392 | −51.0117142 |
| chr14 | 93423461 | 93423464 | 4 | 14:93423461-93423464 | 1.08E−14 | 2.26E−13 | 30.7762175 | 81.2944577 | −50.5182402 |
| chr14 | 93423503 | 93423506 | 4 | 14:93423503-93423506 | 1.08E−14 | 2.26E−13 | 30.7762175 | 81.2944577 | −50.5182402 |
| chr14 | 93423558 | 93423561 | 4 | 14:93423558-93423561 | 1.08E−14 | 2.26E−13 | 30.7762175 | 81.2944577 | −50.5182402 |
| chr14 | 93425210 | 93425213 | 4 | 14:93425210-93425213 | 1.08E−14 | 2.26E−13 | 30.7762175 | 81.2944577 | −50.5182402 |
| chr14 | 93425284 | 93425287 | 4 | 14:93425284-93425287 | 1.08E−14 | 2.26E−13 | 30.7762175 | 81.2944577 | −50.5182402 |
| chr14 | 93425331 | 93425334 | 4 | 14:93425331-93425334 | 1.08E−14 | 2.26E−13 | 30.7762175 | 81.2944577 | −50.5182402 |
| chr14 | 93425520 | 93425523 | 4 | 14:93425520-93425523 | 1.08E−14 | 2.26E−13 | 30.7762175 | 81.2944577 | −50.5182402 |
| chr14 | 93425529 | 93425532 | 4 | 14:93425529-93425532 | 1.08E−14 | 2.26E−13 | 30.7762175 | 81.2944577 | −50.5182402 |
| chr14 | 93425655 | 93425658 | 4 | 14:93425655-93425658 | 1.08E−14 | 2.26E−13 | 30.7762175 | 81.2944577 | −50.5182402 |
| chr14 | 93425687 | 93425691 | 5 | 14:93425687-93425691 | 1.08E−14 | 2.26E−13 | 30.7762175 | 81.2944577 | −50.5182402 |
| chr14 | 93425735 | 93425739 | 5 | 14:93425735-93425739 | 1.08E−14 | 2.26E−13 | 30.7762175 | 81.2944577 | −50.5182402 |
| chr14 | 93425741 | 93425747 | 7 | 14:93425741-93425747 | 1.08E−14 | 2.26E−13 | 30.7762175 | 81.2944577 | −50.5182402 |
| chr14 | 93426221 | 93426224 | 4 | 14:93426221-93426224 | 1.08E−14 | 2.26E−13 | 30.7762175 | 81.2944577 | −50.5182402 |
| chr14 | 93426229 | 93426234 | 6 | 14:93426229-93426234 | 1.08E−14 | 2.26E−13 | 30.7762175 | 81.2944577 | −50.5182402 |
| chr14 | 93426247 | 93426250 | 4 | 14:93426247-93426250 | 1.08E−14 | 2.26E−13 | 30.7762175 | 81.2944577 | −50.5182402 |
| chr14 | 93426257 | 93426260 | 4 | 14:93426257-93426260 | 1.08E−14 | 2.26E−13 | 30.7762175 | 81.2944577 | −50.5182402 |
| chr14 | 93426336 | 93426340 | 5 | 14:93426336-93426340 | 1.08E−14 | 2.26E−13 | 30.7762175 | 81.2944577 | −50.5182402 |
| chr14 | 93426381 | 93426384 | 4 | 14:93426381-93426384 | 1.08E−14 | 2.26E−13 | 30.7762175 | 81.2944577 | −50.5182402 |
| chr14 | 93426456 | 93426459 | 4 | 14:93426456-93426459 | 1.08E−14 | 2.26E−13 | 30.7762175 | 81.2944577 | −50.5182402 |
| chr14 | 93426481 | 93426484 | 4 | 14:93426481-93426484 | 1.08E−14 | 2.26E−13 | 30.7762175 | 81.2944577 | −50.5182402 |
| chr14 | 93426499 | 93426502 | 4 | 14:93426499-93426502 | 1.08E−14 | 2.26E−13 | 30.7762175 | 81.2944577 | −50.5182402 |
| chr14 | 93426875 | 93426878 | 4 | 14:93426875-93426878 | 1.08E−14 | 2.26E−13 | 30.7762175 | 81.2944577 | −50.5182402 |
| chr14 | 93426916 | 93426919 | 4 | 14:93426916-93426919 | 1.08E−14 | 2.26E−13 | 30.7762175 | 81.2944577 | −50.5182402 |
| chr14 | 101158277 | 101158280 | 4 | 14:101158277-101158280 | 5E−07 | 2.51E−06 | 23.8823529 | 66.3684211 | −42.4860681 |
| chr14 | 101158294 | 101158298 | 5 | 14:101158294-101158298 | 5E−07 | 2.51E−06 | 23.8823529 | 66.3684211 | −42.4860681 |
| chr14 | 101159404 | 101159407 | 4 | 14:101159404-101159407 | 5E−07 | 2.51E−06 | 23.8823529 | 66.3684211 | −42.4860681 |
| chr14 | 101159430 | 101159436 | 7 | 14:101159430-101159436 | 5E−07 | 2.51E−06 | 23.8823529 | 66.3684211 | −42.4860681 |
| chr14 | 101159551 | 101159558 | 8 | 14:101159551-101159558 | 5E−07 | 2.51E−06 | 23.8823529 | 66.3684211 | −42.4860681 |
| chr14 | 101250153 | 101250156 | 4 | 14:101250153-101250156 | 0.000291 | 0.000676 | 19.7 | 78.1495405 | −58.4495405 |
| chr14 | 101250165 | 101250168 | 4 | 14:101250165-101250168 | 0.000291 | 0.000676 | 19.7 | 78.1495405 | −58.4495405 |
| chr14 | 101250240 | 101250247 | 8 | 14:101250240-101250247 | 0.000291 | 0.000676 | 19.7 | 78.1495405 | −58.4495405 |
| chr14 | 101250254 | 101250260 | 7 | 14:101250254-101250260 | 0.000291 | 0.000676 | 19.7 | 78.1495405 | −58.4495405 |
| chr14 | 101250279 | 101250282 | 4 | 14:101250279-101250282 | 0.000291 | 0.000676 | 19.7 | 78.1495405 | −58.4495405 |
| chr14 | 101640895 | 101640901 | 7 | 14:101640895-101640901 | 0.000291 | 0.000676 | 7.9 | 70.0902256 | −62.1902256 |
| chr14 | 101640910 | 101640914 | 5 | 14:101640910-101640914 | 0.000291 | 0.000676 | 7.9 | 70.0902256 | −62.1902256 |
| chr14 | 101640942 | 101640946 | 5 | 14:101640942-101640946 | 0.000291 | 0.000676 | 7.9 | 70.0902256 | −62.1902256 |
| chr14 | 101640959 | 101640962 | 4 | 14:101640959-101640962 | 0.000291 | 0.000676 | 7.9 | 70.0902256 | −62.1902256 |
| chr14 | 105347685 | 105347689 | 5 | 14:105347685-105347689 | 1.34E−05 | 4.69E−05 | 28.2871795 | 82.6487854 | −54.3616059 |
| chr14 | 105347700 | 105347703 | 4 | 14:105347700-105347703 | 1.34E−05 | 4.69E−05 | 28.2871795 | 82.6487854 | −54.3616059 |
| chr14 | 105347712 | 105347715 | 4 | 14:105347712-105347715 | 1.34E−05 | 4.69E−05 | 28.2871795 | 82.6487854 | −54.3616059 |
| chr14 | 105347802 | 105347805 | 4 | 14:105347802-105347805 | 1.34E−05 | 4.69E−05 | 28.2871795 | 82.6487854 | −54.3616059 |
| chr14 | 105347810 | 105347814 | 5 | 14:105347810-105347814 | 1.34E−05 | 4.69E−05 | 28.2871795 | 82.6487854 | −54.3616059 |
| chr14 | 105347834 | 105347837 | 4 | 14:105347834-105347837 | 1.34E−05 | 4.69E−05 | 28.2871795 | 82.6487854 | −54.3616059 |
| chr14 | 105347851 | 105347854 | 4 | 14:105347851-105347854 | 1.34E−05 | 4.69E−05 | 28.2871795 | 82.6487854 | −54.3616059 |
| chr14 | 105347961 | 105347965 | 5 | 14:105347961-105347965 | 1.34E−05 | 4.69E−05 | 28.2871795 | 82.6487854 | −54.3616059 |
| chr15 | 22559959 | 22559962 | 4 | 15:22559959-22559962 | 9.61E−08 | 5.72E−07 | 45.1948718 | 85.5502603 | −40.3553885 |
| chr15 | 22559966 | 22559969 | 4 | 15:22559966-22559969 | 9.61E−08 | 5.72E−07 | 45.1948718 | 85.5502603 | −40.3553885 |
| chr15 | 22567152 | 22567155 | 4 | 15:22567152-22567155 | 9.61E−08 | 5.72E−07 | 45.1948718 | 85.5502603 | −40.3553885 |
| chr15 | 22567172 | 22567175 | 4 | 15:22567172-22567175 | 9.61E−08 | 5.72E−07 | 45.1948718 | 85.5502603 | −40.3553885 |
| chr15 | 22571751 | 22571754 | 4 | 15:22571751-22571754 | 9.61E−08 | 5.72E−07 | 45.1948718 | 85.5502603 | −40.3553885 |
| chr15 | 22571783 | 22571786 | 4 | 15:22571783-22571786 | 9.61E−08 | 5.72E−07 | 45.1948718 | 85.5502603 | −40.3553885 |
| chr15 | 22581845 | 22581848 | 4 | 15:22581845-22581848 | 9.61E−08 | 5.72E−07 | 45.1948718 | 85.5502603 | −40.3553885 |
| chr15 | 22581981 | 22581984 | 4 | 15:22581981-22581984 | 9.61E−08 | 5.72E−07 | 45.1948718 | 85.5502603 | −40.3553885 |
| chr15 | 74521143 | 74521146 | 4 | 15:74521143-74521146 | 0.000205 | 0.000508 | 9.05555556 | 58.122807 | −49.0672515 |
| chr15 | 74521156 | 74521159 | 4 | 15:74521156-74521159 | 0.000205 | 0.000508 | 9.05555556 | 58.122807 | −49.0672515 |
| chr15 | 74521297 | 74521300 | 4 | 15:74521297-74521300 | 0.000205 | 0.000508 | 9.05555556 | 58.122807 | −49.0672515 |
| chr15 | 74521344 | 74521348 | 5 | 15:74521344-74521348 | 0.000205 | 0.000508 | 9.05555556 | 58.122807 | −49.0672515 |
| chr15 | 74521357 | 74521360 | 4 | 15:74521357-74521360 | 0.000205 | 0.000508 | 9.05555556 | 58.122807 | −49.0672515 |
| chr15 | 74521364 | 74521367 | 4 | 15:74521364-74521367 | 0.000205 | 0.000508 | 9.05555556 | 58.122807 | −49.0672515 |
| chr15 | 94613752 | 94613758 | 7 | 15:94613752-94613758 | 7.4E−07 | 3.59E−06 | 8.79166667 | 54.9824561 | −46.1907895 |
| chr15 | 94613766 | 94613769 | 4 | 15:94613766-94613769 | 7.4E−07 | 3.59E−06 | 8.79166667 | 54.9824561 | −46.1907895 |
| chr15 | 94613787 | 94613791 | 5 | 15:94613787-94613791 | 7.4E−07 | 3.59E−06 | 8.79166667 | 54.9824561 | −46.1907895 |
| chr15 | 94613823 | 94613829 | 7 | 15:94613823-94613829 | 7.4E−07 | 3.59E−06 | 8.79166667 | 54.9824561 | −46.1907895 |
| chr15 | 94613901 | 94613904 | 4 | 15:94613901-94613904 | 7.4E−07 | 3.59E−06 | 8.79166667 | 54.9824561 | −46.1907895 |
| chr17 | 915606 | 915609 | 4 | 17:915606-915609 | 1.98E−18 | 7.01E−17 | 40.8510684 | 84.8094167 | −43.9583483 |
| chr17 | 915643 | 915646 | 4 | 17:915643-915646 | 1.98E−18 | 7.01E−17 | 40.8510684 | 84.8094167 | −43.9583483 |
| chr17 | 915806 | 915814 | 9 | 17:915806-915814 | 1.98E−18 | 7.01E−17 | 40.8510684 | 84.8094167 | −43.9583483 |
| chr17 | 915890 | 915893 | 4 | 17:915890-915893 | 1.98E−18 | 7.01E−17 | 40.8510684 | 84.8094167 | −43.9583483 |
| chr17 | 915912 | 915915 | 4 | 17:915912-915915 | 1.98E−18 | 7.01E−17 | 40.8510684 | 84.8094167 | −43.9583483 |
| chr17 | 7010397 | 7010400 | 4 | 17:7010397-7010400 | 7.77E−05 | 0.000217 | 16.8875 | 69.8039474 | −52.9164474 |
| chr17 | 7010445 | 7010448 | 4 | 17:7010445-7010448 | 7.77E−05 | 0.000217 | 16.8875 | 69.8039474 | −52.9164474 |
| chr17 | 7010504 | 7010508 | 5 | 17:7010504-7010508 | 7.77E−05 | 0.000217 | 16.8875 | 69.8039474 | −52.9164474 |
| chr17 | 7010533 | 7010536 | 4 | 17:7010533-7010536 | 7.77E−05 | 0.000217 | 16.8875 | 69.8039474 | −52.9164474 |
| chr17 | 7010548 | 7010551 | 4 | 17:7010548-7010551 | 7.77E−05 | 0.000217 | 16.8875 | 69.8039474 | −52.9164474 |
| chr17 | 7010630 | 7010633 | 4 | 17:7010630-7010633 | 7.77E−05 | 0.000217 | 16.8875 | 69.8039474 | −52.9164474 |
| chr17 | 7010663 | 7010666 | 4 | 17:7010663-7010666 | 7.77E−05 | 0.000217 | 16.8875 | 69.8039474 | −52.9164474 |
| chr17 | 10728383 | 10728386 | 4 | 17:10728383-10728386 | 1.34E−07 | 7.74E−07 | 37.4574074 | 81.4695132 | −44.0121058 |
| chr17 | 10740512 | 10740515 | 4 | 17:10740512-10740515 | 1.34E−07 | 7.74E−07 | 37.4574074 | 81.4695132 | −44.0121058 |
| chr17 | 10740558 | 10740566 | 9 | 17:10740558-10740566 | 1.34E−07 | 7.74E−07 | 37.4574074 | 81.4695132 | −44.0121058 |
| chr17 | 10740569 | 10740572 | 4 | 17:10740569-10740572 | 1.34E−07 | 7.74E−07 | 37.4574074 | 81.4695132 | −44.0121058 |
| chr17 | 10740581 | 10740584 | 4 | 17:10740581-10740584 | 1.34E−07 | 7.74E−07 | 37.4574074 | 81.4695132 | −44.0121058 |
| chr17 | 10740595 | 10740598 | 4 | 17:10740595-10740598 | 1.34E−07 | 7.74E−07 | 37.4574074 | 81.4695132 | −44.0121058 |
| chr17 | 17677711 | 17677714 | 4 | 17:17677711-17677714 | 7.71E−07 | 3.7E−06 | 74.2308532 | 32.0229726 | 42.20788061 |
| chr17 | 17677757 | 17677760 | 4 | 17:17677757-17677760 | 7.71E−07 | 3.7E−06 | 74.2308532 | 32.0229726 | 42.20788061 |
| chr17 | 17677887 | 17677890 | 4 | 17:17677887-17677890 | 7.71E−07 | 3.7E−06 | 74.2308532 | 32.0229726 | 42.20788061 |
| chr17 | 21286388 | 21286392 | 5 | 17:21286388-21286392 | 1.74E−08 | 1.22E−07 | 16.3889579 | 57.900155 | −41.5111971 |
| chr17 | 21286444 | 21286447 | 4 | 17:21286444-21286447 | 1.74E−08 | 1.22E−07 | 16.3889579 | 57.900155 | −41.5111971 |
| chr17 | 21286486 | 21286490 | 5 | 17:21286486-21286490 | 1.74E−08 | 1.22E−07 | 16.3889579 | 57.900155 | −41.5111971 |
| chr17 | 21286510 | 21286519 | 10 | 17:21286510-21286519 | 1.74E−08 | 1.22E−07 | 16.3889579 | 57.900155 | −41.5111971 |
| chr17 | 21286534 | 21286538 | 5 | 17:21286534-21286538 | 1.74E−08 | 1.22E−07 | 16.3889579 | 57.900155 | −41.5111971 |
| chr17 | 21286708 | 21286714 | 7 | 17:21286708-21286714 | 1.74E−08 | 1.22E−07 | 16.3889579 | 57.900155 | −41.5111971 |
| chr17 | 21286741 | 21286744 | 4 | 17:21286741-21286744 | 1.74E−08 | 1.22E−07 | 16.3889579 | 57.900155 | −41.5111971 |
| chr17 | 21286747 | 21286750 | 4 | 17:21286747-21286750 | 1.74E−08 | 1.22E−07 | 16.3889579 | 57.900155 | −41.5111971 |
| chr17 | 28299552 | 28299555 | 4 | 17:28299552-28299555 | 9.3E−06 | 3.39E−05 | 24.6793651 | 85.5445354 | −60.8651703 |
| chr17 | 28299565 | 28299568 | 4 | 17:28299565-28299568 | 9.3E−06 | 3.39E−05 | 24.6793651 | 85.5445354 | −60.8651703 |
| chr17 | 28299571 | 28299576 | 6 | 17:28299571-28299576 | 9.3E−06 | 3.39E−05 | 24.6793651 | 85.5445354 | −60.8651703 |
| chr17 | 28299598 | 28299602 | 5 | 17:28299598-28299602 | 9.3E−06 | 3.39E−05 | 24.6793651 | 85.5445354 | −60.8651703 |
| chr17 | 28299697 | 28299700 | 4 | 17:28299697-28299700 | 9.3E−06 | 3.39E−05 | 24.6793651 | 85.5445354 | −60.8651703 |
| chr17 | 71715903 | 71715906 | 4 | 17:71715903-71715906 | 8.71E−05 | 0.000239 | 16.6593122 | 56.997271 | −40.3379588 |
| chr17 | 71715940 | 71715944 | 5 | 17:71715940-71715944 | 8.71E−05 | 0.000239 | 16.6593122 | 56.997271 | −40.3379588 |
| chr17 | 71715947 | 71715951 | 5 | 17:71715947-71715951 | 8.71E−05 | 0.000239 | 16.6593122 | 56.997271 | −40.3379588 |
| chr17 | 71715958 | 71715966 | 9 | 17:71715958-71715966 | 8.71E−05 | 0.000239 | 16.6593122 | 56.997271 | −40.3379588 |
| chr17 | 71716005 | 71716008 | 4 | 17:71716005-71716008 | 8.71E−05 | 0.000239 | 16.6593122 | 56.997271 | −40.3379588 |
| chr17 | 71743873 | 71743876 | 4 | 17:71743873-71743876 | 7.4E−07 | 3.59E−06 | 25.0339616 | 74.2394389 | −49.2054772 |
| chr17 | 71744668 | 71744671 | 4 | 17:71744668-71744671 | 7.4E−07 | 3.59E−06 | 25.0339616 | 74.2394389 | −49.2054772 |
| chr17 | 71744685 | 71744689 | 5 | 17:71744685-71744689 | 7.4E−07 | 3.59E−06 | 25.0339616 | 74.2394389 | −49.2054772 |
| chr17 | 71744723 | 71744727 | 5 | 17:71744723-71744727 | 7.4E−07 | 3.59E−06 | 25.0339616 | 74.2394389 | −49.2054772 |
| chr17 | 71744739 | 71744742 | 4 | 17:71744739-71744742 | 7.4E−07 | 3.59E−06 | 25.0339616 | 74.2394389 | −49.2054772 |
| chr17 | 78079606 | 78079609 | 4 | 17:78079606-78079609 | 7.38E−09 | 5.62E−08 | 17.1 | 58.3221248 | −41.2221248 |
| chr17 | 78079613 | 78079616 | 4 | 17:78079613-78079616 | 7.38E−09 | 5.62E−08 | 17.1 | 58.3221248 | −41.2221248 |
| chr17 | 78079629 | 78079633 | 5 | 17:78079629-78079633 | 7.38E−09 | 5.62E−08 | 17.1 | 58.3221248 | −41.2221248 |
| chr17 | 78079641 | 78079645 | 5 | 17:78079641-78079645 | 7.38E−09 | 5.62E−08 | 17.1 | 58.3221248 | −41.2221248 |
| chr17 | 78079653 | 78079656 | 4 | 17:78079653-78079656 | 7.38E−09 | 5.62E−08 | 17.1 | 58.3221248 | −41.2221248 |
| chr17 | 78395359 | 78395362 | 4 | 17:78395359-78395362 | 4.11E−05 | 0.000125 | 35.7694444 | 76.2602339 | −40.4907895 |
| chr17 | 78395376 | 78395379 | 4 | 17:78395376-78395379 | 4.11E−05 | 0.000125 | 35.7694444 | 76.2602339 | −40.4907895 |
| chr17 | 80314127 | 80314130 | 4 | 17:80314127-80314130 | 4.04E−05 | 0.000124 | 80.5181818 | 32.6555024 | 47.86267943 |
| chr17 | 80314154 | 80314157 | 4 | 17:80314154-80314157 | 4.04E−05 | 0.000124 | 80.5181818 | 32.6555024 | 47.86267943 |
| chr17 | 80914812 | 80914815 | 4 | 17:80914812-80914815 | 1.42E−08 | 1.02E−07 | 51.4227273 | 92.4904306 | −41.0677034 |
| chr17 | 80915007 | 80915010 | 4 | 17:80915007-80915010 | 1.42E−08 | 1.02E−07 | 51.4227273 | 92.4904306 | −41.0677034 |
| chr17 | 80915042 | 80915045 | 4 | 17:80915042-80915045 | 1.42E−08 | 1.02E−07 | 51.4227273 | 92.4904306 | −41.0677034 |
| chr17 | 80915160 | 80915164 | 5 | 17:80915160-80915164 | 1.42E−08 | 1.02E−07 | 51.4227273 | 92.4904306 | −41.0677034 |
| chr17 | 80915190 | 80915193 | 4 | 17:80915190-80915193 | 1.42E−08 | 1.02E−07 | 51.4227273 | 92.4904306 | −41.0677034 |
| chr17 | 80915238 | 80915241 | 4 | 17:80915238-80915241 | 1.42E−08 | 1.02E−07 | 51.4227273 | 92.4904306 | −41.0677034 |
| chr17 | 80915248 | 80915251 | 4 | 17:80915248-80915251 | 1.42E−08 | 1.02E−07 | 51.4227273 | 92.4904306 | −41.0677034 |
| chr17 | 80915270 | 80915276 | 7 | 17:80915270-80915276 | 1.42E−08 | 1.02E−07 | 51.4227273 | 92.4904306 | −41.0677034 |
| chr17 | 80915289 | 80915292 | 4 | 17:80915289-80915292 | 1.42E−08 | 1.02E−07 | 51.4227273 | 92.4904306 | −41.0677034 |
| chr17 | 80915308 | 80915311 | 4 | 17:80915308-80915311 | 1.42E−08 | 1.02E−07 | 51.4227273 | 92.4904306 | −41.0677034 |
| chr18 | 8659649 | 8659656 | 8 | 18:8659649-8659656 | 1.56E−08 | 1.1E−07 | 34.0428571 | 89.5616984 | −55.5188412 |
| chr18 | 24348426 | 24348429 | 4 | 18:24348426-24348429 | 2.06E−05 | 6.77E−05 | 47.2296296 | 88.3160123 | −41.0863826 |
| chr18 | 25366256 | 25366259 | 4 | 18:25366256-25366259 | 1.66E−09 | 1.44E−08 | 28.3402778 | 77.7105263 | −49.3702485 |
| chr18 | 25366262 | 25366266 | 5 | 18:25366262-25366266 | 1.66E−09 | 1.44E−08 | 28.3402778 | 77.7105263 | −49.3702485 |
| chr18 | 25366300 | 25366304 | 5 | 18:25366300-25366304 | 1.66E−09 | 1.44E−08 | 28.3402778 | 77.7105263 | −49.3702485 |
| chr18 | 25366309 | 25366312 | 4 | 18:25366309-25366312 | 1.66E−09 | 1.44E−08 | 28.3402778 | 77.7105263 | −49.3702485 |
| chr18 | 25442175 | 25442178 | 4 | 18:25442175-25442178 | 1.66E−09 | 1.44E−08 | 28.3402778 | 77.7105263 | −49.3702485 |
| chr18 | 25442224 | 25442228 | 5 | 18:25442224-25442228 | 1.66E−09 | 1.44E−08 | 28.3402778 | 77.7105263 | −49.3702485 |
| chr18 | 25442243 | 25442246 | 4 | 18:25442243-25442246 | 1.66E−09 | 1.44E−08 | 28.3402778 | 77.7105263 | −49.3702485 |
| chr18 | 68166868 | 68166871 | 4 | 18:68166868-68166871 | 1.61E−07 | 9.12E−07 | 27.9802469 | 83.27687 | −55.2966231 |
| chr18 | 68166878 | 68166885 | 8 | 18:68166878-68166885 | 1.61E−07 | 9.12E−07 | 27.9802469 | 83.27687 | −55.2966231 |
| chr18 | 77320496 | 77320499 | 4 | 18:77320496-77320499 | 2.42E−09 | 2.03E−08 | 35.9851429 | 79.0973044 | −43.1121616 |
| chr18 | 77320538 | 77320542 | 5 | 18:77320538-77320542 | 2.42E−09 | 2.03E−08 | 35.9851429 | 79.0973044 | −43.1121616 |
| chr18 | 77320583 | 77320586 | 4 | 18:77320583-77320586 | 2.42E−09 | 2.03E−08 | 35.9851429 | 79.0973044 | −43.1121616 |
| chr18 | 77320593 | 77320596 | 4 | 18:77320593-77320596 | 2.42E−09 | 2.03E−08 | 35.9851429 | 79.0973044 | −43.1121616 |
| chr18 | 77320606 | 77320609 | 4 | 18:77320606-77320609 | 2.42E−09 | 2.03E−08 | 35.9851429 | 79.0973044 | −43.1121616 |
| chr18 | 77320672 | 77320675 | 4 | 18:77320672-77320675 | 2.42E−09 | 2.03E−08 | 35.9851429 | 79.0973044 | −43.1121616 |
| chr18 | 77320695 | 77320698 | 4 | 18:77320695-77320698 | 2.42E−09 | 2.03E−08 | 35.9851429 | 79.0973044 | −43.1121616 |
| chr18 | 77320711 | 77320715 | 5 | 18:77320711-77320715 | 2.42E−09 | 2.03E−08 | 35.9851429 | 79.0973044 | −43.1121616 |
| chr18 | 77320739 | 77320742 | 4 | 18:77320739-77320742 | 2.42E−09 | 2.03E−08 | 35.9851429 | 79.0973044 | −43.1121616 |
| chr19 | 883734 | 883737 | 4 | 19:883734-883737 | 1.04E−10 | 1.14E−09 | 44.1096825 | 89.0961921 | −44.9865095 |
| chr19 | 883775 | 883778 | 4 | 19:883775-883778 | 1.04E−10 | 1.14E−09 | 44.1096825 | 89.0961921 | −44.9865095 |
| chr19 | 883830 | 883833 | 4 | 19:883830-883833 | 1.04E−10 | 1.14E−09 | 44.1096825 | 89.0961921 | −44.9865095 |
| chr19 | 883873 | 883880 | 8 | 19:883873-883880 | 1.04E−10 | 1.14E−09 | 44.1096825 | 89.0961921 | −44.9865095 |
| chr19 | 883885 | 883888 | 4 | 19:883885-883888 | 1.04E−10 | 1.14E−09 | 44.1096825 | 89.0961921 | −44.9865095 |
| chr19 | 883994 | 883997 | 4 | 19:883994-883997 | 1.04E−10 | 1.14E−09 | 44.1096825 | 89.0961921 | −44.9865095 |
| chr19 | 884032 | 884036 | 5 | 19:884032-884036 | 1.04E−10 | 1.14E−09 | 44.1096825 | 89.0961921 | −44.9865095 |
| chr19 | 884042 | 884045 | 4 | 19:884042-884045 | 1.04E−10 | 1.14E−09 | 44.1096825 | 89.0961921 | −44.9865095 |
| chr19 | 884057 | 884060 | 4 | 19:884057-884060 | 1.04E−10 | 1.14E−09 | 44.1096825 | 89.0961921 | −44.9865095 |
| chr19 | 884140 | 884143 | 4 | 19:884140-884143 | 1.04E−10 | 1.14E−09 | 44.1096825 | 89.0961921 | −44.9865095 |
| chr19 | 884147 | 884150 | 4 | 19:884147-884150 | 1.04E−10 | 1.14E−09 | 44.1096825 | 89.0961921 | −44.9865095 |
| chr19 | 884172 | 884176 | 5 | 19:884172-884176 | 1.04E−10 | 1.14E−09 | 44.1096825 | 89.0961921 | −44.9865095 |
| chr19 | 884233 | 884236 | 4 | 19:884233-884236 | 1.04E−10 | 1.14E−09 | 44.1096825 | 89.0961921 | −44.9865095 |
| chr19 | 884267 | 884271 | 5 | 19:884267-884271 | 1.04E−10 | 1.14E−09 | 44.1096825 | 89.0961921 | −44.9865095 |
| chr19 | 884346 | 884349 | 4 | 19:884346-884349 | 1.04E−10 | 1.14E−09 | 44.1096825 | 89.0961921 | −44.9865095 |
| chr19 | 2299058 | 2299061 | 4 | 19:2299058-2299061 | 0.000291 | 0.000676 | 10.2238095 | 65.1800215 | −54.956212 |
| chr19 | 2299180 | 2299186 | 7 | 19:2299180-2299186 | 0.000291 | 0.000676 | 10.2238095 | 65.1800215 | −54.956212 |
| chr19 | 2299218 | 2299222 | 5 | 19:2299218-2299222 | 0.000291 | 0.000676 | 10.2238095 | 65.1800215 | −54.956212 |
| chr19 | 2299353 | 2299356 | 4 | 19:2299353-2299356 | 0.000291 | 0.000676 | 10.2238095 | 65.1800215 | −54.956212 |
| chr19 | 2299390 | 2299393 | 4 | 19:2299390-2299393 | 0.000291 | 0.000676 | 10.2238095 | 65.1800215 | −54.956212 |
| chr19 | 4566603 | 4566606 | 4 | 19:4566603-4566606 | 3.7E−07 | 1.91E−06 | 20.775 | 81.3640351 | −60.5890351 |
| chr19 | 4566636 | 4566640 | 5 | 19:4566636-4566640 | 3.7E−07 | 1.91E−06 | 20.775 | 81.3640351 | −60.5890351 |
| chr19 | 4566642 | 4566651 | 10 | 19:4566642-4566651 | 3.7E−07 | 1.91E−06 | 20.775 | 81.3640351 | −60.5890351 |
| chr19 | 4566653 | 4566660 | 8 | 19:4566653-4566660 | 3.7E−07 | 1.91E−06 | 20.775 | 81.3640351 | −60.5890351 |
| chr19 | 9994121 | 9994124 | 4 | 19:9994121-9994124 | 7.03E−06 | 2.63E−05 | 30.4240741 | 81.7585283 | −51.3344542 |
| chr19 | 9994137 | 9994140 | 4 | 19:9994137-9994140 | 7.03E−06 | 2.63E−05 | 30.4240741 | 81.7585283 | −51.3344542 |
| chr19 | 9994208 | 9994211 | 4 | 19:9994208-9994211 | 7.03E−06 | 2.63E−05 | 30.4240741 | 81.7585283 | −51.3344542 |
| chr19 | 9994219 | 9994222 | 4 | 19:9994219-9994222 | 7.03E−06 | 2.63E−05 | 30.4240741 | 81.7585283 | −51.3344542 |
| chr19 | 9994333 | 9994336 | 4 | 19:9994333-9994336 | 7.03E−06 | 2.63E−05 | 30.4240741 | 81.7585283 | −51.3344542 |
| chr19 | 9994349 | 9994352 | 4 | 19:9994349-9994352 | 7.03E−06 | 2.63E−05 | 30.4240741 | 81.7585283 | −51.3344542 |
| chr19 | 13108021 | 13108024 | 4 | 19:13108021-13108024 | 2.11E−21 | 1.11E−19 | 76.1324031 | 35.8777879 | 40.25461514 |
| chr19 | 13108640 | 13108643 | 4 | 19:13108640-13108643 | 2.11E−21 | 1.11E−19 | 76.1324031 | 35.8777879 | 40.25461514 |
| chr19 | 13112778 | 13112781 | 4 | 19:13112778-13112781 | 2.11E−21 | 1.11E−19 | 76.1324031 | 35.8777879 | 40.25461514 |
| chr19 | 13616660 | 13616663 | 4 | 19:13616660-13616663 | 8.88E−29 | 1.09E−26 | 63.4765642 | 18.5280761 | 44.94848803 |
| chr19 | 13616810 | 13616813 | 4 | 19:13616810-13616813 | 8.88E−29 | 1.09E−26 | 63.4765642 | 18.5280761 | 44.94848803 |
| chr19 | 13616818 | 13616821 | 4 | 19:13616818-13616821 | 8.88E−29 | 1.09E−26 | 63.4765642 | 18.5280761 | 44.94848803 |
| chr19 | 13616834 | 13616841 | 8 | 19:13616834-13616841 | 8.88E−29 | 1.09E−26 | 63.4765642 | 18.5280761 | 44.94848803 |
| chr19 | 13616868 | 13616873 | 6 | 19:13616868-13616873 | 8.88E−29 | 1.09E−26 | 63.4765642 | 18.5280761 | 44.94848803 |
| chr19 | 13616879 | 13616882 | 4 | 19:13616879-13616882 | 8.88E−29 | 1.09E−26 | 63.4765642 | 18.5280761 | 44.94848803 |
| chr19 | 13616906 | 13616909 | 4 | 19:13616906-13616909 | 8.88E−29 | 1.09E−26 | 63.4765642 | 18.5280761 | 44.94848803 |
| chr19 | 13616911 | 13616914 | 4 | 19:13616911-13616914 | 8.88E−29 | 1.09E−26 | 63.4765642 | 18.5280761 | 44.94848803 |
| chr19 | 13617010 | 13617013 | 4 | 19:13617010-13617013 | 8.88E−29 | 1.09E−26 | 63.4765642 | 18.5280761 | 44.94848803 |
| chr19 | 13617018 | 13617021 | 4 | 19:13617018-13617021 | 8.88E−29 | 1.09E−26 | 63.4765642 | 18.5280761 | 44.94848803 |
| chr19 | 13617024 | 13617027 | 4 | 19:13617024-13617027 | 8.88E−29 | 1.09E−26 | 63.4765642 | 18.5280761 | 44.94848803 |
| chr19 | 13617029 | 13617033 | 5 | 19:13617029-13617033 | 8.88E−29 | 1.09E−26 | 63.4765642 | 18.5280761 | 44.94848803 |
| chr19 | 13617058 | 13617061 | 4 | 19:13617058-13617061 | 8.88E−29 | 1.09E−26 | 63.4765642 | 18.5280761 | 44.94848803 |
| chr19 | 13617065 | 13617068 | 4 | 19:13617065-13617068 | 8.88E−29 | 1.09E−26 | 63.4765642 | 18.5280761 | 44.94848803 |
| chr19 | 13617070 | 13617080 | 11 | 19:13617070-13617080 | 8.88E−29 | 1.09E−26 | 63.4765642 | 18.5280761 | 44.94848803 |
| chr19 | 13617082 | 13617086 | 5 | 19:13617082-13617086 | 8.88E−29 | 1.09E−26 | 63.4765642 | 18.5280761 | 44.94848803 |
| chr19 | 13617090 | 13617114 | 25 | 19:13617090-13617114 | 8.88E−29 | 1.09E−26 | 63.4765642 | 18.5280761 | 44.94848803 |
| chr19 | 13617128 | 13617132 | 5 | 19:13617128-13617132 | 8.88E−29 | 1.09E−26 | 63.4765642 | 18.5280761 | 44.94848803 |
| chr19 | 13617140 | 13617148 | 9 | 19:13617140-13617148 | 8.88E−29 | 1.09E−26 | 63.4765642 | 18.5280761 | 44.94848803 |
| chr19 | 13617157 | 13617160 | 4 | 19:13617157-13617160 | 8.88E−29 | 1.09E−26 | 63.4765642 | 18.5280761 | 44.94848803 |
| chr19 | 13617163 | 13617166 | 4 | 19:13617163-13617166 | 8.88E−29 | 1.09E−26 | 63.4765642 | 18.5280761 | 44.94848803 |
| chr19 | 13617169 | 13617172 | 4 | 19:13617169-13617172 | 8.88E−29 | 1.09E−26 | 63.4765642 | 18.5280761 | 44.94848803 |
| chr19 | 13617176 | 13617179 | 4 | 19:13617176-13617179 | 8.88E−29 | 1.09E−26 | 63.4765642 | 18.5280761 | 44.94848803 |
| chr19 | 13617194 | 13617203 | 10 | 19:13617194-13617203 | 8.88E−29 | 1.09E−26 | 63.4765642 | 18.5280761 | 44.94848803 |
| chr19 | 13617208 | 13617211 | 4 | 19:13617208-13617211 | 8.88E−29 | 1.09E−26 | 63.4765642 | 18.5280761 | 44.94848803 |
| chr19 | 13617218 | 13617221 | 4 | 19:13617218-13617221 | 8.88E−29 | 1.09E−26 | 63.4765642 | 18.5280761 | 44.94848803 |
| chr19 | 13617233 | 13617236 | 4 | 19:13617233-13617236 | 8.88E−29 | 1.09E−26 | 63.4765642 | 18.5280761 | 44.94848803 |
| chr19 | 13617241 | 13617244 | 4 | 19:13617241-13617244 | 8.88E−29 | 1.09E−26 | 63.4765642 | 18.5280761 | 44.94848803 |
| chr19 | 13617250 | 13617253 | 4 | 19:13617250-13617253 | 8.88E−29 | 1.09E−26 | 63.4765642 | 18.5280761 | 44.94848803 |
| chr19 | 13617265 | 13617268 | 4 | 19:13617265-13617268 | 8.88E−29 | 1.09E−26 | 63.4765642 | 18.5280761 | 44.94848803 |
| chr19 | 13617288 | 13617291 | 4 | 19:13617288-13617291 | 8.88E−29 | 1.09E−26 | 63.4765642 | 18.5280761 | 44.94848803 |
| chr19 | 13617302 | 13617305 | 4 | 19:13617302-13617305 | 8.88E−29 | 1.09E−26 | 63.4765642 | 18.5280761 | 44.94848803 |
| chr19 | 13617315 | 13617318 | 4 | 19:13617315-13617318 | 8.88E−29 | 1.09E−26 | 63.4765642 | 18.5280761 | 44.94848803 |
| chr19 | 13617321 | 13617324 | 4 | 19:13617321-13617324 | 8.88E−29 | 1.09E−26 | 63.4765642 | 18.5280761 | 44.94848803 |
| chr19 | 13617328 | 13617331 | 4 | 19:13617328-13617331 | 8.88E−29 | 1.09E−26 | 63.4765642 | 18.5280761 | 44.94848803 |
| chr19 | 13617336 | 13617339 | 4 | 19:13617336-13617339 | 8.88E−29 | 1.09E−26 | 63.4765642 | 18.5280761 | 44.94848803 |
| chr19 | 13617364 | 13617367 | 4 | 19:13617364-13617367 | 8.88E−29 | 1.09E−26 | 63.4765642 | 18.5280761 | 44.94848803 |
| chr19 | 13617380 | 13617384 | 5 | 19:13617380-13617384 | 8.88E−29 | 1.09E−26 | 63.4765642 | 18.5280761 | 44.94848803 |
| chr19 | 13617419 | 13617422 | 4 | 19:13617419-13617422 | 8.88E−29 | 1.09E−26 | 63.4765642 | 18.5280761 | 44.94848803 |
| chr19 | 13617465 | 13617468 | 4 | 19:13617465-13617468 | 8.88E−29 | 1.09E−26 | 63.4765642 | 18.5280761 | 44.94848803 |
| chr19 | 13617483 | 13617492 | 10 | 19:13617483-13617492 | 8.88E−29 | 1.09E−26 | 63.4765642 | 18.5280761 | 44.94848803 |
| chr19 | 13617503 | 13617506 | 4 | 19:13617503-13617506 | 8.88E−29 | 1.09E−26 | 63.4765642 | 18.5280761 | 44.94848803 |
| chr19 | 13617512 | 13617515 | 4 | 19:13617512-13617515 | 8.88E−29 | 1.09E−26 | 63.4765642 | 18.5280761 | 44.94848803 |
| chr19 | 13617517 | 13617520 | 4 | 19:13617517-13617520 | 8.88E−29 | 1.09E−26 | 63.4765642 | 18.5280761 | 44.94848803 |
| chr19 | 13617541 | 13617544 | 4 | 19:13617541-13617544 | 8.88E−29 | 1.09E−26 | 63.4765642 | 18.5280761 | 44.94848803 |
| chr19 | 13618356 | 13618359 | 4 | 19:13618356-13618359 | 8.88E−29 | 1.09E−26 | 63.4765642 | 18.5280761 | 44.94848803 |
| chr19 | 13618391 | 13618397 | 7 | 19:13618391-13618397 | 8.88E−29 | 1.09E−26 | 63.4765642 | 18.5280761 | 44.94848803 |
| chr19 | 13700379 | 13700382 | 4 | 19:13700379-13700382 | 8.88E−29 | 1.09E−26 | 63.4765642 | 18.5280761 | 44.94848803 |
| chr19 | 18836689 | 18836692 | 4 | 19:18836689-18836692 | 0.000291 | 0.000676 | 22.9015873 | 91.9863722 | −69.0847849 |
| chr19 | 18837043 | 18837049 | 7 | 19:18837043-18837049 | 0.000291 | 0.000676 | 22.9015873 | 91.9863722 | −69.0847849 |
| chr19 | 18837091 | 18837095 | 5 | 19:18837091-18837095 | 0.000291 | 0.000676 | 22.9015873 | 91.9863722 | −69.0847849 |
| chr19 | 18837098 | 18837101 | 4 | 19:18837098-18837101 | 0.000291 | 0.000676 | 22.9015873 | 91.9863722 | −69.0847849 |
| chr19 | 18837105 | 18837108 | 4 | 19:18837105-18837108 | 0.000291 | 0.000676 | 22.9015873 | 91.9863722 | −69.0847849 |
| chr19 | 34181556 | 34181559 | 4 | 19:34181556-34181559 | 0.000291 | 0.000676 | 37.6428571 | 81.6616541 | −44.018797 |
| chr19 | 34181606 | 34181609 | 4 | 19:34181606-34181609 | 0.000291 | 0.000676 | 37.6428571 | 81.6616541 | −44.018797 |
| chr19 | 34181636 | 34181639 | 4 | 19:34181636-34181639 | 0.000291 | 0.000676 | 37.6428571 | 81.6616541 | −44.018797 |
| chr19 | 38527887 | 38527890 | 4 | 19:38527887-38527890 | 2.06E−05 | 6.77E−05 | 26.8814815 | 83.1950847 | −56.3136032 |
| chr19 | 38527913 | 38527916 | 4 | 19:38527913-38527916 | 2.06E−05 | 6.77E−05 | 26.8814815 | 83.1950847 | −56.3136032 |
| chr19 | 38527932 | 38527935 | 4 | 19:38527932-38527935 | 2.06E−05 | 6.77E−05 | 26.8814815 | 83.1950847 | −56.3136032 |
| chr19 | 38528016 | 38528019 | 4 | 19:38528016-38528019 | 2.06E−05 | 6.77E−05 | 26.8814815 | 83.1950847 | −56.3136032 |
| chr19 | 38528031 | 38528038 | 8 | 19:38528031-38528038 | 2.06E−05 | 6.77E−05 | 26.8814815 | 83.1950847 | −56.3136032 |
| chr19 | 38684235 | 38684238 | 4 | 19:38684235-38684238 | 4.06E−05 | 0.000124 | 30.9323232 | 89.3702818 | −58.4379585 |
| chr19 | 38684327 | 38684330 | 4 | 19:38684327-38684330 | 4.06E−05 | 0.000124 | 30.9323232 | 89.3702818 | −58.4379585 |
| chr19 | 38684338 | 38684341 | 4 | 19:38684338-38684341 | 4.06E−05 | 0.000124 | 30.9323232 | 89.3702818 | −58.4379585 |
| chr19 | 38684363 | 38684373 | 11 | 19:38684363-38684373 | 4.06E−05 | 0.000124 | 30.9323232 | 89.3702818 | −58.4379585 |
| chr19 | 48699558 | 48699561 | 4 | 19:48699558-48699561 | 3.52E−07 | 1.85E−06 | 21.4178105 | 72.374785 | −50.9569745 |
| chr19 | 48699578 | 48699582 | 5 | 19:48699578-48699582 | 3.52E−07 | 1.85E−06 | 21.4178105 | 72.374785 | −50.9569745 |
| chr19 | 48699588 | 48699592 | 5 | 19:48699588-48699592 | 3.52E−07 | 1.85E−06 | 21.4178105 | 72.374785 | −50.9569745 |
| chr19 | 48699615 | 48699618 | 4 | 19:48699615-48699618 | 3.52E−07 | 1.85E−06 | 21.4178105 | 72.374785 | −50.9569745 |
| chr19 | 48699628 | 48699632 | 5 | 19:48699628-48699632 | 3.52E−07 | 1.85E−06 | 21.4178105 | 72.374785 | −50.9569745 |
| chr19 | 48699667 | 48699673 | 7 | 19:48699667-48699673 | 3.52E−07 | 1.85E−06 | 21.4178105 | 72.374785 | −50.9569745 |
| chr19 | 48699717 | 48699720 | 4 | 19:48699717-48699720 | 3.52E−07 | 1.85E−06 | 21.4178105 | 72.374785 | −50.9569745 |
| chr19 | 55870392 | 55870395 | 4 | 19:55870392-55870395 | 1.22E−07 | 7.09E−07 | 35.0189096 | 76.1367913 | −41.1178817 |
| chr19 | 55870402 | 55870405 | 4 | 19:55870402-55870405 | 1.22E−07 | 7.09E−07 | 35.0189096 | 76.1367913 | −41.1178817 |
| chr19 | 55870407 | 55870411 | 5 | 19:55870407-55870411 | 1.22E−07 | 7.09E−07 | 35.0189096 | 76.1367913 | −41.1178817 |
| chr19 | 55870425 | 55870429 | 5 | 19:55870425-55870429 | 1.22E−07 | 7.09E−07 | 35.0189096 | 76.1367913 | −41.1178817 |
| chr19 | 55870439 | 55870443 | 5 | 19:55870439-55870443 | 1.22E−07 | 7.09E−07 | 35.0189096 | 76.1367913 | −41.1178817 |
| chr19 | 55870456 | 55870460 | 5 | 19:55870456-55870460 | 1.22E−07 | 7.09E−07 | 35.0189096 | 76.1367913 | −41.1178817 |
| chr19 | 55870462 | 55870465 | 4 | 19:55870462-55870465 | 1.22E−07 | 7.09E−07 | 35.0189096 | 76.1367913 | −41.1178817 |
| chr19 | 55870471 | 55870474 | 4 | 19:55870471-55870474 | 1.22E−07 | 7.09E−07 | 35.0189096 | 76.1367913 | −41.1178817 |
| chr19 | 55870886 | 55870889 | 4 | 19:55870886-55870889 | 1.22E−07 | 7.09E−07 | 35.0189096 | 76.1367913 | −41.1178817 |
| chr20 | 20180688 | 20180693 | 6 | 20:20180688-20180693 | 1.78E−06 | 7.74E−06 | 28.7262626 | 72.8066853 | −44.0804227 |
| chr20 | 20180732 | 20180735 | 4 | 20:20180732-20180735 | 1.78E−06 | 7.74E−06 | 28.7262626 | 72.8066853 | −44.0804227 |
| chr20 | 20188098 | 20188101 | 4 | 20:20188098-20188101 | 1.78E−06 | 7.74E−06 | 28.7262626 | 72.8066853 | −44.0804227 |
| chr20 | 20188151 | 20188154 | 4 | 20:20188151-20188154 | 1.78E−06 | 7.74E−06 | 28.7262626 | 72.8066853 | −44.0804227 |
| chr20 | 20206077 | 20206080 | 4 | 20:20206077-20206080 | 1.78E−06 | 7.74E−06 | 28.7262626 | 72.8066853 | −44.0804227 |
| chr20 | 20232312 | 20232315 | 4 | 20:20232312-20232315 | 1.78E−06 | 7.74E−06 | 28.7262626 | 72.8066853 | −44.0804227 |
| chr20 | 20232336 | 20232339 | 4 | 20:20232336-20232339 | 1.78E−06 | 7.74E−06 | 28.7262626 | 72.8066853 | −44.0804227 |
| chr20 | 20232350 | 20232357 | 8 | 20:20232350-20232357 | 1.78E−06 | 7.74E−06 | 28.7262626 | 72.8066853 | −44.0804227 |
| chr20 | 20232360 | 20232364 | 5 | 20:20232360-20232364 | 1.78E−06 | 7.74E−06 | 28.7262626 | 72.8066853 | −44.0804227 |
| chr20 | 20232370 | 20232373 | 4 | 20:20232370-20232373 | 1.78E−06 | 7.74E−06 | 28.7262626 | 72.8066853 | −44.0804227 |
| chr20 | 20232391 | 20232394 | 4 | 20:20232391-20232394 | 1.78E−06 | 7.74E−06 | 28.7262626 | 72.8066853 | −44.0804227 |
| chr20 | 20376947 | 20376950 | 4 | 20:20376947-20376950 | 1.75E−17 | 5.49E−16 | 29.2188428 | 77.9025833 | −48.6837405 |
| chr20 | 20376962 | 20376965 | 4 | 20:20376962-20376965 | 1.75E−17 | 5.49E−16 | 29.2188428 | 77.9025833 | −48.6837405 |
| chr20 | 20377074 | 20377077 | 4 | 20:20377074-20377077 | 1.75E−17 | 5.49E−16 | 29.2188428 | 77.9025833 | −48.6837405 |
| chr20 | 20377089 | 20377093 | 5 | 20:20377089-20377093 | 1.75E−17 | 5.49E−16 | 29.2188428 | 77.9025833 | −48.6837405 |
| chr20 | 20377110 | 20377113 | 4 | 20:20377110-20377113 | 1.75E−17 | 5.49E−16 | 29.2188428 | 77.9025833 | −48.6837405 |
| chr20 | 20377122 | 20377126 | 5 | 20:20377122-20377126 | 1.75E−17 | 5.49E−16 | 29.2188428 | 77.9025833 | −48.6837405 |
| chr20 | 20377130 | 20377134 | 5 | 20:20377130-20377134 | 1.75E−17 | 5.49E−16 | 29.2188428 | 77.9025833 | −48.6837405 |
| chr20 | 20377162 | 20377165 | 4 | 20:20377162-20377165 | 1.75E−17 | 5.49E−16 | 29.2188428 | 77.9025833 | −48.6837405 |
| chr20 | 20377169 | 20377172 | 4 | 20:20377169-20377172 | 1.75E−17 | 5.49E−16 | 29.2188428 | 77.9025833 | −48.6837405 |
| chr20 | 20377206 | 20377209 | 4 | 20:20377206-20377209 | 1.75E−17 | 5.49E−16 | 29.2188428 | 77.9025833 | −48.6837405 |
| chr20 | 20392139 | 20392149 | 11 | 20:20392139-20392149 | 1.75E−17 | 5.49E−16 | 29.2188428 | 77.9025833 | −48.6837405 |
| chr20 | 20392184 | 20392188 | 5 | 20:20392184-20392188 | 1.75E−17 | 5.49E−16 | 29.2188428 | 77.9025833 | −48.6837405 |
| chr20 | 20392215 | 20392218 | 4 | 20:20392215-20392218 | 1.75E−17 | 5.49E−16 | 29.2188428 | 77.9025833 | −48.6837405 |
| chr20 | 20392408 | 20392411 | 4 | 20:20392408-20392411 | 1.75E−17 | 5.49E−16 | 29.2188428 | 77.9025833 | −48.6837405 |
| chr20 | 20421295 | 20421298 | 4 | 20:20421295-20421298 | 1.75E−17 | 5.49E−16 | 29.2188428 | 77.9025833 | −48.6837405 |
| chr20 | 20421314 | 20421317 | 4 | 20:20421314-20421317 | 1.75E−17 | 5.49E−16 | 29.2188428 | 77.9025833 | −48.6837405 |
| chr20 | 20421345 | 20421349 | 5 | 20:20421345-20421349 | 1.75E−17 | 5.49E−16 | 29.2188428 | 77.9025833 | −48.6837405 |
| chr20 | 20421371 | 20421378 | 8 | 20:20421371-20421378 | 1.75E−17 | 5.49E−16 | 29.2188428 | 77.9025833 | −48.6837405 |
| chr20 | 20421396 | 20421399 | 4 | 20:20421396-20421399 | 1.75E−17 | 5.49E−16 | 29.2188428 | 77.9025833 | −48.6837405 |
| chr20 | 20421420 | 20421425 | 6 | 20:20421420-20421425 | 1.75E−17 | 5.49E−16 | 29.2188428 | 77.9025833 | −48.6837405 |
| chr20 | 20421459 | 20421462 | 4 | 20:20421459-20421462 | 1.75E−17 | 5.49E−16 | 29.2188428 | 77.9025833 | −48.6837405 |
| chr20 | 20421486 | 20421489 | 4 | 20:20421486-20421489 | 1.75E−17 | 5.49E−16 | 29.2188428 | 77.9025833 | −48.6837405 |
| chr20 | 31129481 | 31129484 | 4 | 20:31129481-31129484 | 0.000205 | 0.000508 | 38.1444444 | 86.4619883 | −48.3175439 |
| chr20 | 36923352 | 36923355 | 4 | 20:36923352-36923355 | 0.000247 | 0.000601 | 38.815873 | 90.5694885 | −51.7536155 |
| chr20 | 36923369 | 36923378 | 10 | 20:36923369-36923378 | 0.000247 | 0.000601 | 38.815873 | 90.5694885 | −51.7536155 |
| chr20 | 36923397 | 36923400 | 4 | 20:36923397-36923400 | 0.000247 | 0.000601 | 38.815873 | 90.5694885 | −51.7536155 |
| chr20 | 61013054 | 61013057 | 4 | 20:61013054-61013057 | 1.38E−08 | 9.91E−08 | 31.3136243 | 75.1492959 | −43.8356716 |
| chr20 | 61013094 | 61013097 | 4 | 20:61013094-61013097 | 1.38E−08 | 9.91E−08 | 31.3136243 | 75.1492959 | −43.8356716 |
| chr20 | 61013113 | 61013116 | 4 | 20:61013113-61013116 | 1.38E−08 | 9.91E−08 | 31.3136243 | 75.1492959 | −43.8356716 |
| chr20 | 61013148 | 61013152 | 5 | 20:61013148-61013152 | 1.38E−08 | 9.91E−08 | 31.3136243 | 75.1492959 | −43.8356716 |
| chr20 | 61013167 | 61013171 | 5 | 20:61013167-61013171 | 1.38E−08 | 9.91E−08 | 31.3136243 | 75.1492959 | −43.8356716 |
| chr20 | 61013187 | 61013194 | 8 | 20:61013187-61013194 | 1.38E−08 | 9.91E−08 | 31.3136243 | 75.1492959 | −43.8356716 |
| chr20 | 61013226 | 61013230 | 5 | 20:61013226-61013230 | 1.38E−08 | 9.91E−08 | 31.3136243 | 75.1492959 | −43.8356716 |
| chr20 | 61013242 | 61013245 | 4 | 20:61013242-61013245 | 1.38E−08 | 9.91E−08 | 31.3136243 | 75.1492959 | −43.8356716 |
| chr20 | 61013251 | 61013254 | 4 | 20:61013251-61013254 | 1.38E−08 | 9.91E−08 | 31.3136243 | 75.1492959 | −43.8356716 |
| chr20 | 61013263 | 61013266 | 4 | 20:61013263-61013266 | 1.38E−08 | 9.91E−08 | 31.3136243 | 75.1492959 | −43.8356716 |
| chr20 | 61013303 | 61013306 | 4 | 20:61013303-61013306 | 1.38E−08 | 9.91E−08 | 31.3136243 | 75.1492959 | −43.8356716 |
| chr20 | 61013339 | 61013344 | 6 | 20:61013339-61013344 | 1.38E−08 | 9.91E−08 | 31.3136243 | 75.1492959 | −43.8356716 |
| chr20 | 61013474 | 61013477 | 4 | 20:61013474-61013477 | 1.38E−08 | 9.91E−08 | 31.3136243 | 75.1492959 | −43.8356716 |
| chr20 | 61013515 | 61013519 | 5 | 20:61013515-61013519 | 1.38E−08 | 9.91E−08 | 31.3136243 | 75.1492959 | −43.8356716 |
| chr20 | 61020520 | 61020523 | 4 | 20:61020520-61020523 | 1.38E−08 | 9.91E−08 | 31.3136243 | 75.1492959 | −43.8356716 |
| chr20 | 61021747 | 61021750 | 4 | 20:61021747-61021750 | 1.38E−08 | 9.91E−08 | 31.3136243 | 75.1492959 | −43.8356716 |
| chr21 | 43442046 | 43442049 | 4 | 21:43442046-43442049 | 7.03E−06 | 2.63E−05 | 37.0657407 | 79.291552 | −42.2258113 |
| chr21 | 43442055 | 43442058 | 4 | 21:43442055-43442058 | 7.03E−06 | 2.63E−05 | 37.0657407 | 79.291552 | −42.2258113 |
| chr21 | 43442083 | 43442086 | 4 | 21:43442083-43442086 | 7.03E−06 | 2.63E−05 | 37.0657407 | 79.291552 | −42.2258113 |
| chr21 | 43442252 | 43442255 | 4 | 21:43442252-43442255 | 7.03E−06 | 2.63E−05 | 37.0657407 | 79.291552 | −42.2258113 |
| chr21 | 43442338 | 43442341 | 4 | 21:43442338-43442341 | 7.03E−06 | 2.63E−05 | 37.0657407 | 79.291552 | −42.2258113 |
| chr21 | 43442384 | 43442387 | 4 | 21:43442384-43442387 | 7.03E−06 | 2.63E−05 | 37.0657407 | 79.291552 | −42.2258113 |
| chr21 | 44836763 | 44836766 | 4 | 21:44836763-44836766 | 2.57E−09 | 2.15E−08 | 42.4082437 | 85.4872969 | −43.0790531 |
| chr21 | 44836797 | 44836800 | 4 | 21:44836797-44836800 | 2.57E−09 | 2.15E−08 | 42.4082437 | 85.4872969 | −43.0790531 |
| chr21 | 144836823 | 44836826 | 4 | 21:44836823-44836826 | 2.57E−09 | 2.15E−08 | 42.4082437 | 85.4872969 | −43.0790531 |
| chr21 | 44836842 | 44836845 | 4 | 21:44836842-44836845 | 2.57E−09 | 2.15E−08 | 42.4082437 | 85.4872969 | −43.0790531 |
| chr21 | 44836924 | 44836927 | 4 | 21:44836924-44836927 | 2.57E−09 | 2.15E−08 | 42.4082437 | 85.4872969 | −43.0790531 |
| chr21 | 44836936 | 44836939 | 4 | 21:44836936-44836939 | 2.57E−09 | 2.15E−08 | 42.4082437 | 85.4872969 | −43.0790531 |
| chr21 | 44836971 | 44836974 | 4 | 21:44836971-44836974 | 2.57E−09 | 2.15E−08 | 42.4082437 | 85.4872969 | −43.0790531 |
| chr21 | 44837026 | 44837036 | 11 | 21:44837026-44837036 | 2.57E−09 | 2.15E−08 | 42.4082437 | 85.4872969 | −43.0790531 |
| chr21 | 44837044 | 44837047 | 4 | 21:44837044-44837047 | 2.57E−09 | 2.15E−08 | 42.4082437 | 85.4872969 | −43.0790531 |
| chr21 | 44837052 | 44837055 | 4 | 21:44837052-44837055 | 2.57E−09 | 2.15E−08 | 42.4082437 | 85.4872969 | −43.0790531 |
| chr21 | 44837060 | 44837063 | 4 | 21:44837060-44837063 | 2.57E−09 | 2.15E−08 | 42.4082437 | 85.4872969 | −43.0790531 |
| chr21 | 44837074 | 44837081 | 8 | 21:44837074-44837081 | 2.57E−09 | 2.15E−08 | 42.4082437 | 85.4872969 | −43.0790531 |
| chr21 | 44837115 | 44837118 | 4 | 21:44837115-44837118 | 2.57E−09 | 2.15E−08 | 42.4082437 | 85.4872969 | −43.0790531 |
| chr21 | 44837132 | 44837137 | 6 | 21:44837132-44837137 | 2.57E−09 | 2.15E−08 | 42.4082437 | 85.4872969 | −43.0790531 |
| chr21 | 44837157 | 44837160 | 4 | 21:44837157-44837160 | 2.57E−09 | 2.15E−08 | 42.4082437 | 85.4872969 | −43.0790531 |
| chr21 | 145653981 | 45653984 | 4 | 21:45653981-45653984 | 1.82E−05 | 6.16E−05 | 40.9645833 | 90.0250348 | −49.0604514 |
| chr21 | 45654156 | 45654159 | 4 | 21:45654156-45654159 | 1.82E−05 | 6.16E−05 | 40.9645833 | 90.0250348 | −49.0604514 |
| chr22 | 47444997 | 47445000 | 4 | 22:47444997-47445000 | 1.48E−05 | 5.17E−05 | 42.8794643 | 83.3530016 | −40.4735374 |
| chr22 | 47445037 | 47445040 | 4 | 22:47445037-47445040 | 1.48E−05 | 5.17E−05 | 42.8794643 | 83.3530016 | −40.4735374 |
| chr22 | 47447406 | 47447409 | 4 | 22:47447406-47447409 | 1.48E−05 | 5.17E−05 | 42.8794643 | 83.3530016 | −40.4735374 |
| chr22 | 47447603 | 47447606 | 4 | 22:47447603-47447606 | 1.48E−05 | 5.17E−05 | 42.8794643 | 83.3530016 | −40.4735374 |
| chrX | 104458760 | 104458763 | 4 | X:104458760-104458763 | 3.84E−10 | 3.77E−09 | 30.1677821 | 70.4658778 | −40.2980958 |
| chrX | 104692650 | 104692653 | 4 | X:104692650-104692653 | 3.84E−10 | 3.77E−09 | 30.1677821 | 70.4658778 | −40.2980958 |
| chrX | 104804571 | 104804574 | 4 | X:104804571-104804574 | 3.84E−10 | 3.77E−09 | 30.1677821 | 70.4658778 | −40.2980958 |
| chrX | 104891135 | 104891138 | 4 | X:104891135-104891138 | 3.84E−10 | 3.77E−09 | 30.1677821 | 70.4658778 | −40.2980958 |
| chrX | 105016634 | 105016637 | 4 | X:105016634-105016637 | 3.84E−10 | 3.77E−09 | 30.1677821 | 70.4658778 | −40.2980958 |
| chrX | 105016672 | 105016675 | 4 | X:105016672-105016675 | 3.84E−10 | 3.77E−09 | 30.1677821 | 70.4658778 | −40.2980958 |
| chrX | 128926432 | 128926435 | 4 | X:128926432-128926435 | 7.77E−05 | 0.000217 | 47.9 | 90.3212719 | −42.4212719 |
| chr2 | 209103955 | 209103984 | 30 | 2:209103955-209103984 | | | | | |
| chr4 | 106155150 | 106155179 | 30 | 4:106155150-106155179 | | | | | |
| chr4 | 106155913 | 106155942 | 30 | 4:106155913-106155942 | | | | | |
| chr4 | 106156460 | 106156489 | 30 | 4:106156460-106156489 | | | | | |
| chr4 | 106156742 | 106156771 | 30 | 4:106156742-106156771 | | | | | |
| chr4 | 106157415 | 106157444 | 30 | 4:106157415-106157444 | | | | | |
| chr4 | 106164901 | 106164930 | 30 | 4:106164901-106164930 | | | | | |
| chr4 | 106196236 | 106196265 | 30 | 4:106196236-106196265 | | | | | |
| chr7 | 33732332 | 33732392 | 61 | 7:33732332-33732392 | | | | | |
| chr7 | 140453119 | 140453148 | 30 | 7:140453119-140453148 | | | | | |
| chr7 | 140481387 | 140481416 | 30 | 7:140481387-140481416 | | | | | |
| chr7 | 140481426 | 140481455 | 30 | 7:140481426-140481455 | | | | | |
| chr7 | 140500421 | 140500450 | 30 | 7:140500421-140500450 | | | | | |
| chr7 | 140507748 | 140507777 | 30 | 7:140507748-140507777 | | | | | |
| chr7 | 140507954 | 140507983 | 30 | 7:140507954-140507983 | | | | | |
| chr7 | 140624441 | 140624470 | 30 | 7:140624441-140624470 | | | | | |
| chr14 | 38061176 | 38061227 | 52 | 14:38061176-38061227 | | | | | |
| chr14 | 38061319 | 38061348 | 30 | 14:38061319-38061348 | | | | | |
| chr14 | 38061830 | 38061859 | 30 | 14:38061830-38061859 | | | | | |
| chr17 | 7572948 | 7572977 | 30 | 17:7572948-7572977 | | | | | |
| chr17 | 7573967 | 7574049 | 83 | 17:7573967-7574049 | | | | | |
| chr17 | 7576524 | 7576555 | 32 | 17:7576524-7576555 | | | | | |
| chr17 | 7576838 | 7576942 | 105 | 17:7576838-7576942 | | | | | |
| chr17 | 7577020 | 7577156 | 137 | 17:7577020-7577156 | | | | | |
| chr17 | 7577483 | 7577624 | 142 | 17:7577483-7577624 | | | | | |
| chr17 | 7578161 | 7578289 | 129 | 17:7578161-7578289 | | | | | |
| chr17 | 7578373 | 7578570 | 198 | 17:7578373-7578570 | | | | | |
| chr17 | 7579297 | 7579363 | 67 | 17:7579297-7579363 | | | | | |
| chr17 | 7579393 | 7579431 | 39 | 17:7579393-7579431 | | | | | |
| chr17 | 7579506 | 7579535 | 30 | 17:7579506-7579535 | | | | | |
| chr17 | 7579701 | 7579730 | 30 | 17:7579701-7579730 | | | | | |
| chr17 | 7579846 | 7579875 | 30 | 17:7579846-7579875 | | | | | |
| chr17 | 47696409 | 47696463 | 55 | 17:47696409-47696463 | | | | | |
| chr17 | 47696673 | 47696702 | 30 | 17:47696673-47696702 | | | | | |
| chr20 | 31375142 | 31375171 | 30 | 20:31375142-31375171 | | | | | |
| chr20 | 31380384 | 31380413 | 30 | 20:31380384-31380413 | | | | | |
| chr20 | 31381300 | 31381329 | 30 | 20:31381300-31381329 | | | | | |
| chr20 | 31386255 | 31386284 | 30 | 20:31386255-31386284 | | | | | |
| chr20 | 31388050 | 31388079 | 30 | 20:31388050-31388079 | | | | | |
| chr20 | 31389120 | 31389149 | 30 | 20:31389120-31389149 | | | | | |
| chr20 | 31393185 | 31393214 | 30 | 20:31393185-31393214 | | | | | |
| chr20 | 31395662 | 31395691 | 30 | 20:31395662-31395691 | | | | | |
| chrX | 66121000 | 66123000 | 2001 | X:66121000-66123000 | | | | | |
| chrX | 66766332 | 66766361 | 30 | X:66766332-66766361 | | | | | |
| chrX | 66931448 | 66931477 | 30 | X:66931448-66931477 | | | | | |
| chrX | 66937357 | 66937386 | 30 | X:66937357-66937386 | | | | | |
| chrX | 66943528 | 66943566 | 39 | X:66943528-66943566 | | | | | |
| chr2 | 25452399 | 25452408 | 10 | 2:25452399-25452408 | | | | | −44.6842105 |
| chr2 | 25452969 | 25452978 | 10 | 2:25452969-25452978 | | | | | −46.3947368 |
| chr2 | 25453099 | 25453108 | 10 | 2:25453099-25453108 | | | | | −56.4578947 |
| chr2 | 25453105 | 25453114 | 10 | 2:25453105-25453114 | | | | | −61.8777778 |
| chr2 | 25453799 | 25453808 | 10 | 2:25453799-25453808 | | | | | −67.0157895 |
| chr2 | 25453898 | 25453907 | 10 | 2:25453898-25453907 | | | | | −52.9736842 |
| chr2 | 25453899 | 25453908 | 10 | 2:25453899-25453908 | | | | | −59.1473684 |
| chr2 | 25453913 | 25453922 | 10 | 2:25453913-25453922 | | | | | −52.4210526 |
| chr2 | 25499982 | 25499991 | 10 | 2:25499982-25499991 | | | | | 42.11111111 |
| chr2 | 25500271 | 25500280 | 10 | 2:25500271-25500280 | | | | | 49.31052632 |
| chr2 | 25500272 | 25500281 | 10 | 2:25500272-25500281 | | | | | 43.20526316 |
| chr2 | 239147428 | 239147437 | 10 | 2:239147428-239147437 | | | | | −58.4 |
| chr2 | 239147440 | 239147449 | 10 | 2:239147440-239147449 | | | | | −55.4555556 |
| chr2 | 239147441 | 239147450 | 10 | 2:239147441-239147450 | | | | | −64.5555556 |
| chr2 | 239147485 | 239147494 | 10 | 2:239147485-239147494 | | | | | −57.2666667 |
| chr2 | 239147486 | 239147495 | 10 | 2:239147486-239147495 | | | | | −63 |
| chr2 | 239147522 | 239147531 | 10 | 2:239147522-239147531 | | | | | −53.6222222 |
| chr2 | 239147534 | 239147543 | 10 | 2:239147534-239147543 | | | | | −63.9888889 |
| chr2 | 239147589 | 239147598 | 10 | 2:239147589-239147598 | | | | | −56.5777778 |
| chr2 | 239147601 | 239147610 | 10 | 2:239147601-239147610 | | | | | −55.0222222 |
| chr2 | 239147614 | 239147623 | 10 | 2:239147614-239147623 | | | | | −48.7666667 |
| chr2 | 239147637 | 239147646 | 10 | 2:239147637-239147646 | | | | | −52.5529412 |
| chr2 | 239147649 | 239147658 | 10 | 2:239147649-239147658 | | | | | −61.5705882 |
| chr2 | 239147665 | 239147674 | 10 | 2:239147665-239147674 | | | | | −54.4235294 |
| chr2 | 239149894 | 239149903 | 10 | 2:239149894-239149903 | | | | | −53.7888889 |
| chr2 | 239149895 | 239149904 | 10 | 2:239149895-239149904 | | | | | −43.8 |
| chr3 | 12838468 | 12838477 | 10 | 3:12838468-12838477 | | | | | −41.9052632 |
| chr3 | 50660608 | 50660617 | 10 | 3:50660608-50660617 | | | | | 44.50526316 |
| chr3 | 50670155 | 50670164 | 10 | 3:50670155-50670164 | | | | | 40.1031746 |
| chr3 | 128202781 | 128202790 | 10 | 3:128202781-128202790 | | | | | 45.50877193 |
| chr3 | 128202787 | 128202796 | 10 | 3:128202787-128202796 | | | | | 48.78362573 |
| chr3 | 128202792 | 128202801 | 10 | 3:128202792-128202801 | | | | | 41.28654971 |
| chr3 | 128202795 | 128202804 | 10 | 3:128202795-128202804 | | | | | 43.26315789 |
| chr3 | 128202796 | 128202805 | 10 | 3:128202796-128202805 | | | | | 44.72222222 |
| chr3 | 128202884 | 128202893 | 10 | 3:128202884-128202893 | | | | | 40.23976608 |
| chr3 | 128202894 | 128202903 | 10 | 3:128202894-128202903 | | | | | 43.92982456 |
| chr3 | 128202895 | 128202904 | 10 | 3:128202895-128202904 | | | | | 45.03508772 |
| chr3 | 128202927 | 128202936 | 10 | 3:128202927-128202936 | | | | | 51.92982456 |
| chr3 | 128202928 | 128202937 | 10 | 3:128202928-128202937 | | | | | 43.60233918 |
| chr3 | 128205157 | 128205166 | 10 | 3:128205157-128205166 | | | | | 53.52631579 |
| chr3 | 128205179 | 128205188 | 10 | 3:128205179-128205188 | | | | | 55.15037594 |
| chr3 | 128205180 | 128205189 | 10 | 3:128205180-128205189 | | | | | 40.60526316 |
| chr3 | 128205195 | 128205204 | 10 | 3:128205195-128205204 | | | | | 48.94852941 |
| chr3 | 128205196 | 128205205 | 10 | 3:128205196-128205205 | | | | | 45.20394737 |
| chr3 | 128205210 | 128205219 | 10 | 3:128205210-128205219 | | | | | 54.09558824 |
| chr3 | 128210723 | 128210732 | 10 | 3:128210723-128210732 | | | | | 40.07368421 |
| chr3 | 128210727 | 128210736 | 10 | 3:128210727-128210736 | | | | | 42.18947368 |
| chr3 | 128212207 | 128212216 | 10 | 3:128212207-128212216 | | | | | 40.71052632 |
| chr3 | 185817004 | 185817013 | 10 | 3:185817004-185817013 | | | | | −52.0947368 |
| chr4 | 16084875 | 16084884 | 10 | 4:16084875-16084884 | | | | | 43.16842105 |
| chr4 | 16084881 | 16084890 | 10 | 4:16084881-16084890 | | | | | 43.45789474 |
| chr4 | 16084898 | 16084907 | 10 | 4:16084898-16084907 | | | | | 48.74736842 |
| chr4 | 16085260 | 16085269 | 10 | 4:16085260-16085269 | | | | | 40.82222222 |
| chr4 | 16085380 | 16085389 | 10 | 4:16085380-16085389 | | | | | 45.40789474 |
| chr4 | 16085396 | 16085405 | 10 | 4:16085396-16085405 | | | | | 44.39473684 |
| chr4 | 16085398 | 16085407 | 10 | 4:16085398-16085407 | | | | | 40.30921053 |
| chr4 | 16085401 | 16085410 | 10 | 4:16085401-16085410 | | | | | 43.36184211 |
| chr5 | 172661872 | 172661881 | 10 | 5:172661872-172661881 | | | | | 44.85294118 |
| chr5 | 172661874 | 172661883 | 10 | 5:172661874-172661883 | | | | | 44.38970588 |
| chr6 | 34522044 | 34522053 | 10 | 6:34522044-34522053 | | | | | 45.2 |
| chr6 | 34523223 | 34523232 | 10 | 6:34523223-34523232 | | | | | 49.2748538 |
| chr8 | 11561726 | 11561735 | 10 | 8:11561726-11561735 | | | | | 46.85714286 |
| chr8 | 11565855 | 11565864 | 10 | 8:11565855-11565864 | | | | | 40.16666667 |
| chr8 | 11566229 | 11566238 | 10 | 8:11566229-11566238 | | | | | 54.66071429 |
| chr8 | 11583613 | 11583622 | 10 | 8:11583613-11583622 | | | | | −49.5823529 |
| chr8 | 11583614 | 11583623 | 10 | 8:11583614-11583623 | | | | | −46.3888889 |
| chr9 | 126775063 | 126775072 | 10 | 9:126775063-126775072 | | | | | 43.06666667 |
| chr9 | 126775082 | 126775091 | 10 | 9:126775082-126775091 | | | | | 40.71428571 |
| chr9 | 126776013 | 126776022 | 10 | 9:126776013-126776022 | | | | | 41.57777778 |
| chr9 | 126776231 | 126776240 | 10 | 9:126776231-126776240 | | | | | 43.23026316 |
| chr9 | 139437635 | 139437644 | 10 | 9:139437635-139437644 | | | | | −41.3947368 |
| chr9 | 139437679 | 139437688 | 10 | 9:139437679-139437688 | | | | | −43.5473684 |
| chr10 | 23481414 | 23481423 | 10 | 10:23481414-23481423 | | | | | 40.9005848 |
| chr10 | 23481415 | 23481424 | 10 | 10:23481415-23481424 | | | | | 54.35087719 |
| chr10 | 23481417 | 23481426 | 10 | 10:23481417-23481426 | | | | | 40.70175439 |
| chr10 | 23481445 | 23481454 | 10 | 10:23481445-23481454 | | | | | 40.52631579 |
| chr10 | 23481447 | 23481456 | 10 | 10:23481447-23481456 | | | | | 40.65497076 |
| chr10 | 23481448 | 23481457 | 10 | 10:23481448-23481457 | | | | | 45.71052632 |
| chr10 | 23481451 | 23481460 | 10 | 10:23481451-23481460 | | | | | 40.93157895 |
| chr10 | 23481461 | 23481470 | 10 | 10:23481461-23481470 | | | | | 40.32105263 |
| chr10 | 23481462 | 23481471 | 10 | 10:23481462-23481471 | | | | | 47.26973684 |
| chr10 | 23481463 | 23481472 | 10 | 10:23481463-23481472 | | | | | 54.58947368 |
| chr10 | 56699066 | 56699075 | 10 | 10:56699066-56699075 | | | | | −46.7222222 |
| chr10 | 56699077 | 56699086 | 10 | 10:56699077-56699086 | | | | | −42.7777778 |
| chr10 | 57391015 | 57391024 | 10 | 10:57391015-57391024 | | | | | 41.7593985 |
| chr11 | 69451450 | 69451459 | 10 | 11:69451450-69451459 | | | | | 45.03921569 |
| chr12 | 103344993 | 103345002 | 10 | 12:103344993-103345002 | | | | | −44.7936508 |
| chr12 | 103344998 | 103345007 | 10 | 12:103344998-103345007 | | | | | −62.6466165 |
| chr12 | 103345006 | 103345015 | 10 | 12:103345006-103345015 | | | | | −62.0451128 |
| chr12 | 103345029 | 103345038 | 10 | 12:103345029-103345038 | | | | | −60.7669173 |
| chr12 | 103345033 | 103345042 | 10 | 12:103345033-103345042 | | | | | −53.631579 |
| chr14 | 51560040 | 51560049 | 10 | 14:51560040-51560049 | | | | | −62.8105263 |
| chr14 | 51560162 | 51560171 | 10 | 14:51560162-51560171 | | | | | −50.431579 |
| chr14 | 51560191 | 51560200 | 10 | 14:51560191-51560200 | | | | | −40.2736842 |
| chr14 | 51560193 | 51560202 | 10 | 14:51560193-51560202 | | | | | −45.4052632 |
| chr14 | 51560208 | 51560217 | 10 | 14:51560208-51560217 | | | | | −42.2578947 |
| chr14 | 51560259 | 51560268 | 10 | 14:51560259-51560268 | | | | | −46.4842105 |
| chr14 | 51560261 | 51560270 | 10 | 14:51560261-51560270 | | | | | −43.8105263 |
| chr14 | 51560275 | 51560284 | 10 | 14:51560275-51560284 | | | | | −43.3894737 |
| chr14 | 65409361 | 65409370 | 10 | 14:65409361-65409370 | | | | | −46.9 |
| chr14 | 93385930 | 93385939 | 10 | 14:93385930-93385939 | | | | | 53.35555556 |
| chr14 | 93423499 | 93423508 | 10 | 14:93423499-93423508 | | | | | −42.4473684 |
| chr14 | 93423555 | 93423564 | 10 | 14:93423555-93423564 | | | | | −43.6421053 |
| chr14 | 93425158 | 93425167 | 10 | 14:93425158-93425167 | | | | | −46.0842105 |
| chr14 | 93425189 | 93425198 | 10 | 14:93425189-93425198 | | | | | −40.6111111 |
| chr14 | 93425207 | 93425216 | 10 | 14:93425207-93425216 | | | | | −52.5333333 |
| chr14 | 93425289 | 93425298 | 10 | 14:93425289-93425298 | | | | | −50.2473684 |
| chr14 | 93425328 | 93425337 | 10 | 14:93425328-93425337 | | | | | −61.0105263 |
| chr14 | 93425517 | 93425526 | 10 | 14:93425517-93425526 | | | | | −75.7444444 |
| chr14 | 93425684 | 93425693 | 10 | 14:93425684-93425693 | | | | | −71.2263158 |
| chr14 | 93425685 | 93425694 | 10 | 14:93425685-93425694 | | | | | −69.3294118 |
| chr14 | 93425738 | 93425747 | 10 | 14:93425738-93425747 | | | | | −74.2368421 |
| chr14 | 93426226 | 93426235 | 10 | 14:93426226-93426235 | | | | | −50.0473684 |
| chr14 | 93426244 | 93426253 | 10 | 14:93426244-93426253 | | | | | −55.9947368 |
| chr14 | 93426333 | 93426342 | 10 | 14:93426333-93426342 | | | | | −51.4947368 |
| chr14 | 93426378 | 93426387 | 10 | 14:93426378-93426387 | | | | | −50.4052632 |
| chr14 | 93426872 | 93426881 | 10 | 14:93426872-93426881 | | | | | −45.3294118 |
| chr14 | 93426913 | 93426922 | 10 | 14:93426913-93426922 | | | | | −50.5079365 |
| chr14 | 93426914 | 93426923 | 10 | 14:93426914-93426923 | | | | | −40.5411765 |
| chr14 | 93426959 | 93426968 | 10 | 14:93426959-93426968 | | | | | −43.8421053 |
| chr14 | 93439910 | 93439919 | 10 | 14:93439910-93439919 | | | | | 47.32105263 |
| chr18 | 31323189 | 31323198 | 10 | 18:31323189-31323198 | | | | | −54.5666667 |
| chr18 | 31323202 | 31323211 | 10 | 18:31323202-31323211 | | | | | −44.3 |
| chr18 | 53175324 | 53175333 | 10 | 18:53175324-53175333 | | | | | −42.8680556 |
| chr18 | 53305364 | 53305373 | 10 | 18:53305364-53305373 | | | | | −48.8736842 |
| chr18 | 53305490 | 53305499 | 10 | 18:53305490-53305499 | | | | | −40.0555556 |
| chr19 | 11086495 | 11086504 | 10 | 19:11086495-11086504 | | | | | 51.29473684 |
| chr19 | 39993532 | 39993541 | 10 | 19:39993532-39993541 | | | | | 42.04210526 |
| chr19 | 39993551 | 39993560 | 10 | 19:39993551-39993560 | | | | | 40.98947368 |
| chr19 | 39993567 | 39993576 | 10 | 19:39993567-39993576 | | | | | 41.53157895 |
| chr19 | 39993606 | 39993615 | 10 | 19:39993606-39993615 | | | | | 46.69078947 |
| chr19 | 42618585 | 42618594 | 10 | 19:42618585-42618594 | | | | | −46.4947368 |
| chr19 | 42618760 | 42618769 | 10 | 19:42618760-42618769 | | | | | −45.8947368 |
| chr19 | 42618787 | 42618796 | 10 | 19:42618787-42618796 | | | | | −53.9210526 |
| chr19 | 42618789 | 42618798 | 10 | 19:42618789-42618798 | | | | | −53.5052632 |
| chr19 | 42618823 | 42618832 | 10 | 19:42618823-42618832 | | | | | −43.8842105 |
| chr20 | 20232309 | 20232318 | 10 | 20:20232309-20232318 | | | | | −47.1 |
| chr20 | 20232319 | 20232328 | 10 | 20:20232319-20232328 | | | | | −48 |
| chr20 | 20232333 | 20232342 | 10 | 20:20232333-20232342 | | | | | −45.7105263 |
| chr20 | 20232347 | 20232356 | 10 | 20:20232347-20232356 | | | | | −49.6210526 |
| chr20 | 20232348 | 20232357 | 10 | 20:20232348-20232357 | | | | | −51.1578947 |
| chr20 | 20232351 | 20232360 | 10 | 20:20232351-20232360 | | | | | −55.8105263 |
| chr20 | 20232352 | 20232361 | 10 | 20:20232352-20232361 | | | | | −44.8473684 |
| chr20 | 20232357 | 20232366 | 10 | 20:20232357-20232366 | | | | | −56.5736842 |
| chr20 | 20232358 | 20232367 | 10 | 20:20232358-20232367 | | | | | −52.6894737 |
| chr20 | 20232367 | 20232376 | 10 | 20:20232367-20232376 | | | | | −54.831579 |
| chr20 | 20232388 | 20232397 | 10 | 20:20232388-20232397 | | | | | −53.1526316 |
| chr20 | 20266415 | 20266424 | 10 | 20:20266415-20266424 | | | | | 47.07368421 |
| chr20 | 20376954 | 20376963 | 10 | 20:20376954-20376963 | | | | | −59.3666667 |
| chr20 | 20376969 | 20376978 | 10 | 20:20376969-20376978 | | | | | −61.7222222 |
| chr20 | 20377086 | 20377095 | 10 | 20:20377086-20377095 | | | | | −64.0368421 |
| chr20 | 20377158 | 20377167 | 10 | 20:20377158-20377167 | | | | | −60.7361111 |
| chr20 | 20377165 | 20377174 | 10 | 20:20377165-20377174 | | | | | −58.0588235 |
| chr20 | 20421292 | 20421301 | 10 | 20:20421292-20421301 | | | | | −69.0789474 |
| chr20 | 20421311 | 20421320 | 10 | 20:20421311-20421320 | | | | | −72.3842105 |
| chr20 | 20421456 | 20421465 | 10 | 20:20421456-20421465 | | | | | −67.9842105 |
| chr20 | 20421483 | 20421492 | 10 | 20:20421483-20421492 | | | | | −41.9842105 |
| chr20 | 20433160 | 20433169 | 10 | 20:20433160-20433169 | | | | | −46.5263158 |
| chr20 | 20433163 | 20433172 | 10 | 20:20433163-20433172 | | | | | −49.5947368 |
| chr20 | 20433166 | 20433175 | 10 | 20:20433166-20433175 | | | | | −49.9736842 |
| chr20 | 20433198 | 20433207 | 10 | 20:20433198-20433207 | | | | | −48.1368421 |
| chr20 | 20433208 | 20433217 | 10 | 20:20433208-20433217 | | | | | −49.0578947 |
| chr20 | 20433248 | 20433257 | 10 | 20:20433248-20433257 | | | | | −43.7578947 |
| chr20 | 20433256 | 20433265 | 10 | 20:20433256-20433265 | | | | | −47.3263158 |
| chr20 | 20433263 | 20433272 | 10 | 20:20433263-20433272 | | | | | −44.9736842 |
| chr20 | 20433284 | 20433293 | 10 | 20:20433284-20433293 | | | | | −45.2 |
| chr20 | 20433293 | 20433302 | 10 | 20:20433293-20433302 | | | | | −44.6473684 |
| chr20 | 21490704 | 21490713 | 10 | 20:21490704-21490713 | | | | | 47.75555556 |
| chr20 | 21490799 | 21490808 | 10 | 20:21490799-21490808 | | | | | 44.52678571 |
| chr20 | 21490807 | 21490816 | 10 | 20:21490807-21490816 | | | | | 42.02678571 |
| chr20 | 21494139 | 21494148 | 10 | 20:21494139-21494148 | | | | | 46.82142857 |
| chr20 | 21494141 | 21494150 | 10 | 20:21494141-21494150 | | | | | 45.1031746 |
| chr20 | 21494237 | 21494246 | 10 | 20:21494237-21494246 | | | | | 41.92857143 |
| chr21 | 22564429 | 22564438 | 10 | 21:22564429-22564438 | | | | | −41.6368421 |
| chr21 | 42850711 | 42850720 | 10 | 21:42850711-42850720 | | | | | 41.23157895 |
| chr21 | 42860322 | 42860331 | 10 | 21:42860322-42860331 | | | | | 43.55555556 |
| chr21 | 42860341 | 42860350 | 10 | 21:42860341-42860350 | | | | | 48.27777778 |
| chr21 | 42880610 | 42880619 | 10 | 21:42880610-42880619 | | | | | 55.7 |
| chr21 | 42880614 | 42880623 | 10 | 21:42880614-42880623 | | | | | 53.05263158 |
| chr21 | 42880659 | 42880668 | 10 | 21:42880659-42880668 | | | | | 51.56842105 |
| chr21 | 42880668 | 42880677 | 10 | 21:42880668-42880677 | | | | | 52.91578947 |
| chr1 | 3052478 | 3052487 | 10 | 1:3052478-3052487 | | | | | −76.5789474 |
| chr1 | 26735081 | 26735090 | 10 | 1:26735081-26735090 | | | | | 73.14444444 |
| chr1 | 26735241 | 26735250 | 10 | 1:26735241-26735250 | | | | | 75.64052288 |
| chr1 | 26735243 | 26735252 | 10 | 1:26735243-26735252 | | | | | 78 |
| chr1 | 26735251 | 26735260 | 10 | 1:26735251-26735260 | | | | | 80.5 |
| chr1 | 65721361 | 65721370 | 10 | 1:65721361-65721370 | | | | | −78.5079365 |
| chr1 | 229195756 | 229195765 | 10 | 1:229195756-229195765 | | | | | 72.40833333 |
| chr2 | 23807028 | 23807037 | 10 | 2:23807028-23807037 | | | | | −77.3526316 |
| chr2 | 171679652 | 171679661 | 10 | 2:171679652-171679661 | | | | | 70.68055556 |
| chr2 | 236877206 | 236877215 | 10 | 2:236877206-236877215 | | | | | −76.2235294 |
| chr2 | 242549633 | 242549642 | 10 | 2:242549633-242549642 | | | | | 76.84558824 |
| chr2 | 242549635 | 242549644 | 10 | 2:242549635-242549644 | | | | | 70.26470588 |
| chr2 | 242549638 | 242549647 | 10 | 2:242549638-242549647 | | | | | 75.72794118 |
| chr2 | 242549659 | 242549668 | 10 | 2:242549659-242549668 | | | | | 71.69117647 |
| chr3 | 26664523 | 26664532 | 10 | 3:26664523-26664532 | | | | | 70.5380117 |
| chr3 | 126664525 | 26664534 | 10 | 3:26664525-26664534 | | | | | 72.15789474 |
| chr3 | 111431556 | 111431565 | 10 | 3:111431556-111431565 | | | | | −78.0666667 |
| chr4 | 169799044 | 169799053 | 10 | 4:169799044-169799053 | | | | | 73.85714286 |
| chr5 | 138441347 | 138441356 | 10 | 5:138441347-138441356 | | | | | −77.3333333 |
| chr5 | 138441374 | 138441383 | 10 | 5:138441374-138441383 | | | | | −77.9532164 |
| chr5 | 138441376 | 138441385 | 10 | 5:138441376-138441385 | | | | | −77.1907895 |
| chr5 | 176038510 | 176038519 | 10 | 5:176038510-176038519 | | | | | 84.52631579 |
| chr5 | 176038524 | 176038533 | 10 | 5:176038524-176038533 | | | | | 84.57894737 |
| chr5 | 176038525 | 176038534 | 10 | 5:176038525-176038534 | | | | | 83.23391813 |
| chr5 | 176038562 | 176038571 | 10 | 5:176038562-176038571 | | | | | 84.13450292 |
| chr5 | 179401799 | 179401808 | 10 | 5:179401799-179401808 | | | | | −76.2748538 |
| chr6 | 15265454 | 15265463 | 10 | 6:15265454-15265463 | | | | | 72.38970588 |
| chr6 | 168462402 | 168462411 | 10 | 6:168462402-168462411 | | | | | −78.6470588 |
| chr6 | 168462447 | 168462456 | 10 | 6:168462447-168462456 | | | | | −76.7916667 |
| chr6 | 170578588 | 170578597 | 10 | 6:170578588-170578597 | | | | | −77.3222222 |
| chr7 | 2968311 | 2968320 | 10 | 7:2968311-2968320 | | | | | −82.4285714 |
| chr7 | 2968441 | 2968450 | 10 | 7:2968441-2968450 | | | | | −81.5723684 |
| chr7 | 2968442 | 2968451 | 10 | 7:2968442-2968451 | | | | | −78.1052632 |
| chr7 | 2968451 | 2968460 | 10 | 7:2968451-2968460 | | | | | −81.625 |
| chr7 | 2968454 | 2968463 | 10 | 7:2968454-2968463 | | | | | −78.3881579 |
| chr7 | 5572006 | 5572015 | 10 | 7:5572006-5572015 | | | | | 71.00735294 |
| chr7 | 150812220 | 150812229 | 10 | 7:150812220-150812229 | | | | | 70.96031746 |
| chr8 | 47570600 | 47570609 | 10 | 8:47570600-47570609 | | | | | −76.5073529 |
| chr9 | 116411412 | 116411421 | 10 | 9:116411412-116411421 | | | | | −77.6527778 |
| chr9 | 124498726 | 124498735 | 10 | 9:124498726-124498735 | | | | | 72.78571429 |
| chr9 | 124620057 | 124620066 | 10 | 9:124620057-124620066 | | | | | −77.4027778 |
| chr10 | 676777 | 676786 | 10 | 10:676777-676786 | | | | | −77.8235294 |
| chr10 | 676811 | 676820 | 10 | 10:676811-676820 | | | | | −77.2916667 |
| chr11 | 2000638 | 2000647 | 10 | 11:2000638-2000647 | | | | | −76.2222222 |
| chr11 | 11600192 | 11600201 | 10 | 11:11600192-11600201 | | | | | −81.2698413 |
| chr11 | 11600210 | 11600219 | 10 | 11:11600210-11600219 | | | | | −76.484127 |
| chr11 | 11600228 | 11600237 | 10 | 11:11600228-11600237 | | | | | −76.531746 |
| chr11 | 11600230 | 11600239 | 10 | 11:11600230-11600239 | | | | | −79.7410714 |
| chr11 | 11600231 | 11600240 | 10 | 11:11600231-11600240 | | | | | −78.1111111 |
| chr11 | 11600240 | 11600249 | 10 | 11:11600240-11600249 | | | | | −85.2222222 |
| chr11 | 64120601 | 64120610 | 10 | 11:64120601-64120610 | | | | | −76.25 |
| chr11 | 70851727 | 70851736 | 10 | 11:70851727-70851736 | | | | | −76.0368421 |
| chr12 | 40618451 | 40618460 | 10 | 12:40618451-40618460 | | | | | 70.66666667 |
| chr12 | 40618493 | 40618502 | 10 | 12:40618493-40618502 | | | | | 75.59663866 |
| chr15 | 32385689 | 32385698 | 10 | 15:32385689-32385698 | | | | | −75.7894737 |
| chr15 | 68723747 | 68723756 | 10 | 15:68723747-68723756 | | | | | 71.30357143 |
| chr15 | 89954601 | 89954610 | 10 | 15:89954601-89954610 | | | | | 70.32894737 |
| chr15 | 89954603 | 89954612 | 10 | 15:89954603-89954612 | | | | | 73.43421053 |
| chr15 | 89954605 | 89954614 | 10 | 15:89954605-89954614 | | | | | 76.00657895 |
| chr15 | 89959942 | 89959951 | 10 | 15:89959942-89959951 | | | | | 75.5625 |
| chr16 | 85498200 | 85498209 | 10 | 16:85498200-85498209 | | | | | −84.7647059 |
| chr16 | 85498219 | 85498228 | 10 | 16:85498219-85498228 | | | | | −86.7460318 |
| chr16 | 85498220 | 85498229 | 10 | 16:85498220-85498229 | | | | | −78.1805556 |
| chr16 | 85498227 | 85498236 | 10 | 16:85498227-85498236 | | | | | −77.8095238 |
| chr16 | 85498781 | 85498790 | 10 | 16:85498781-85498790 | | | | | −76.0095238 |
| chr16 | 85519155 | 85519164 | 10 | 16:85519155-85519164 | | | | | −83.3216374 |
| chr16 | 85519162 | 85519171 | 10 | 16:85519162-85519171 | | | | | −83.122807 |
| chr16 | 85519165 | 85519174 | 10 | 16:85519165-85519174 | | | | | −86.3508772 |
| chr16 | 85519168 | 85519177 | 10 | 16:85519168-85519177 | | | | | −85.8245614 |
| chr16 | 85519171 | 85519180 | 10 | 16:85519171-85519180 | | | | | −84.0584795 |
| chr16 | 85519173 | 85519182 | 10 | 16:85519173-85519182 | | | | | −86.6608187 |
| chr16 | 85519303 | 85519312 | 10 | 16:85519303-85519312 | | | | | −79.7777778 |
| chr16 | 88715953 | 88715962 | 10 | 16:88715953-88715962 | | | | | −78.1517857 |
| chr17 | 28299568 | 28299577 | 10 | 17:28299568-28299577 | | | | | −75.8666667 |
| chr17 | 80372745 | 80372754 | 10 | 17:80372745-80372754 | | | | | 80.13888889 |
| chr17 | 80372760 | 80372769 | 10 | 17:80372760-80372769 | | | | | 77.36111111 |
| chr17 | 80372761 | 80372770 | 10 | 17:80372761-80372770 | | | | | 81.15789474 |
| chr17 | 80372766 | 80372775 | 10 | 17:80372766-80372775 | | | | | 81.81944444 |
| chr17 | 80372767 | 80372776 | 10 | 17:80372767-80372776 | | | | | 78.36090226 |
| chr17 | 80372770 | 80372779 | 10 | 17:80372770-80372779 | | | | | 74.01388889 |
| chr17 | 80372771 | 80372780 | 10 | 17:80372771-80372780 | | | | | 73.78947368 |
| chr17 | 80372793 | 80372802 | 10 | 17:80372793-80372802 | | | | | 73.14285714 |
| chr18 | 57364080 | 57364089 | 10 | 18:57364080-57364089 | | | | | 74.94152047 |
| chr19 | 591647 | 591656 | 10 | 19:591647-591656 | | | | | 74.02521008 |
| chr19 | 591656 | 591665 | 10 | 19:591656-591665 | | | | | 75.22222222 |
| chr19 | 591660 | 591669 | 10 | 19:591660-591669 | | | | | 72.11904762 |
| chr19 | 591662 | 591671 | 10 | 19:591662-591671 | | | | | 72.12698413 |
| chr19 | 591682 | 591691 | 10 | 19:591682-591691 | | | | | 73.85714286 |
| chr19 | 591683 | 591692 | 10 | 19:591683-591692 | | | | | 79.97619048 |
| chr19 | 591684 | 591693 | 10 | 19:591684-591693 | | | | | 72.21428571 |
| chr19 | 591685 | 591694 | 10 | 19:591685-591694 | | | | | 79.30252101 |
| chr19 | 591688 | 591697 | 10 | 19:591688-591697 | | | | | 78.18253968 |
| chr19 | 18837089 | 18837098 | 10 | 19:18837089-18837098 | | | | | −76.3263158 |
| chr19 | 18837102 | 18837111 | 10 | 19:18837102-18837111 | | | | | −75.6894737 |
| chr20 | 3778162 | 3778171 | 10 | 20:3778162-3778171 | | | | | −78.0684211 |
| chr20 | 32156432 | 32156441 | 10 | 20:32156432-32156441 | | | | | −79.8 |
| chr20 | 48099327 | 48099336 | 10 | 20:48099327-48099336 | | | | | 70.24444444 |
| chr20 | 48099339 | 48099348 | 10 | 20:48099339-48099348 | | | | | 75.36666667 |
| chrX | 36981727 | 36981736 | 10 | X:36981727-36981736 | | | | | 73.33552632 |
| chrX | 36981729 | 36981738 | 10 | X:36981729-36981738 | | | | | 72.09868421 |
| chr1 | 24392969 | 24393169 | 201 | 1:24392969-24393169 | | | | | |
| chr1 | 24410522 | 24410722 | 201 | 1:24410522-24410722 | | | | | |
| chr1 | 24434829 | 24435029 | 201 | 1:24434829-24435029 | | | | | |
| chr1 | 24438565 | 24438765 | 201 | 1:24438565-24438765 | | | | | |
| chr1 | 27240357 | 27240557 | 201 | 1:27240357-27240557 | | | | | |
| chr1 | 50513586 | 50513786 | 201 | 1:50513586-50513786 | | | | | |
| chr1 | 50569482 | 50569682 | 201 | 1:50569482-50569682 | | | | | |
| chr1 | 50571262 | 50571462 | 201 | 1:50571262-50571462 | | | | | |
| chr1 | 50571864 | 50572064 | 201 | 1:50571864-50572064 | | | | | |
| chr1 | 50574505 | 50574705 | 201 | 1:50574505-50574705 | | | | | |
| chr1 | 50574538 | 50574738 | 201 | 1:50574538-50574738 | | | | | |
| chr1 | 50575198 | 50575398 | 201 | 1:50575198-50575398 | | | | | |
| chr1 | 50575278 | 50575478 | 201 | 1:50575278-50575478 | | | | | |
| chr1 | 50575388 | 50575588 | 201 | 1:50575388-50575588 | | | | | |
| chr1 | 50610525 | 50610725 | 201 | 1:50610525-50610725 | | | | | |
| chr1 | 50642702 | 50642902 | 201 | 1:50642702-50642902 | | | | | |
| chr1 | 78445126 | 78445326 | 201 | 1:78445126-78445326 | | | | | |
| chr1 | 78511486 | 78511686 | 201 | 1:78511486-78511686 | | | | | |
| chr1 | 114521963 | 114522163 | 201 | 1:114521963-114522163 | | | | | |
| chr1 | 114521965 | 114522165 | 201 | 1:114521965-114522165 | | | | | |
| chr1 | 114522013 | 114522213 | 201 | 1:114522013-114522213 | | | | | |
| chr1 | 114522595 | 114522795 | 201 | 1:114522595-114522795 | | | | | |
| chr1 | 152956457 | 152956657 | 201 | 1:152956457-152956657 | | | | | |
| chr1 | 152957607 | 152957807 | 201 | 1:152957607-152957807 | | | | | |
| chr1 | 153330230 | 153330430 | 201 | 1:153330230-153330430 | | | | | |
| chr2 | 74119341 | 74119541 | 201 | 2:74119341-74119541 | | | | | |
| chr2 | 74119978 | 74120178 | 201 | 2:74119978-74120178 | | | | | |
| chr2 | 74119994 | 74120194 | 201 | 2:74119994-74120194 | | | | | |
| chr2 | 74120006 | 74120206 | 201 | 2:74120006-74120206 | | | | | |
| chr2 | 74120009 | 74120209 | 201 | 2:74120009-74120209 | | | | | |
| chr2 | 74120035 | 74120235 | 201 | 2:74120035-74120235 | | | | | |
| chr2 | 95940101 | 95940301 | 201 | 2:95940101-95940301 | | | | | |
| chr2 | 95940121 | 95940321 | 201 | 2:95940121-95940321 | | | | | |
| chr2 | 95940140 | 95940340 | 201 | 2:95940140-95940340 | | | | | |
| chr2 | 95940145 | 95940345 | 201 | 2:95940145-95940345 | | | | | |
| chr2 | 95940149 | 95940349 | 201 | 2:95940149-95940349 | | | | | |
| chr2 | 95940627 | 95940827 | 201 | 2:95940627-95940827 | | | | | |
| chr2 | 95943464 | 95943664 | 201 | 2:95943464-95943664 | | | | | |
| chr2 | 95945514 | 95945714 | 201 | 2:95945514-95945714 | | | | | |
| chr2 | 95954155 | 95954355 | 201 | 2:95954155-95954355 | | | | | |
| chr2 | 95954163 | 95954363 | 201 | 2:95954163-95954363 | | | | | |
| chr2 | 169921199 | 169921399 | 201 | 2:169921199-169921399 | | | | | |
| chr2 | 169923445 | 169923645 | 201 | 2:169923445-169923645 | | | | | |
| chr2 | 169923487 | 169923687 | 201 | 2:169923487-169923687 | | | | | |
| chr2 | 169925966 | 169926166 | 201 | 2:169925966-169926166 | | | | | |
| chr2 | 169925972 | 169926172 | 201 | 2:169925972-169926172 | | | | | |
| chr2 | 169925986 | 169926186 | 201 | 2:169925986-169926186 | | | | | |
| chr2 | 169928910 | 169929110 | 201 | 2:169928910-169929110 | | | | | |
| chr2 | 169937517 | 169937717 | 201 | 2:169937517-169937717 | | | | | |
| chr2 | 177001240 | 177001440 | 201 | 2:177001240-177001440 | | | | | |
| chr2 | 177001585 | 177001785 | 201 | 2:177001585-177001785 | | | | | |
| chr2 | 177015022 | 177015222 | 201 | 2:177015022-177015222 | | | | | |
| chr2 | 177025519 | 177025719 | 201 | 2:177025519-177025719 | | | | | |
| chr2 | 177028705 | 177028905 | 201 | 2:177028705-177028905 | | | | | |
| chr2 | 182545174 | 182545374 | 201 | 2:182545174-182545374 | | | | | |
| chr2 | 182545503 | 182545703 | 201 | 2:182545503-182545703 | | | | | |
| chr2 | 219696414 | 219696614 | 201 | 2:219696414-219696614 | | | | | |
| chr2 | 219696424 | 219696624 | 201 | 2:219696424-219696624 | | | | | |
| chr2 | 219696426 | 219696626 | 201 | 2:219696426-219696626 | | | | | |
| chr2 | 219696439 | 219696639 | 201 | 2:219696439-219696639 | | | | | |
| chr2 | 219696709 | 219696909 | 201 | 2:219696709-219696909 | | | | | |
| chr2 | 223916432 | 223916632 | 201 | 2:223916432-223916632 | | | | | |
| chr2 | 223916762 | 223916962 | 201 | 2:223916762-223916962 | | | | | |
| chr2 | 223916818 | 223917018 | 201 | 2:223916818-223917018 | | | | | |
| chr2 | 233924577 | 233924777 | 201 | 2:233924577-233924777 | | | | | |
| chr2 | 233924962 | 233925162 | 201 | 2:233924962-233925162 | | | | | |
| chr2 | 233925089 | 233925289 | 201 | 2:233925089-233925289 | | | | | |
| chr2 | 233940872 | 233941072 | 201 | 2:233940872-233941072 | | | | | |
| chr2 | 233985212 | 233985412 | 201 | 2:233985212-233985412 | | | | | |
| chr2 | 233986826 | 233987026 | 201 | 2:233986826-233987026 | | | | | |
| chr2 | 234053819 | 234054019 | 201 | 2:234053819-234054019 | | | | | |
| chr2 | 234054695 | 234054895 | 201 | 2:234054695-234054895 | | | | | |
| chr2 | 234070270 | 234070470 | 201 | 2:234070270-234070470 | | | | | |
| chr2 | 234070335 | 234070535 | 201 | 2:234070335-234070535 | | | | | |
| chr2 | 234072847 | 234073047 | 201 | 2:234072847-234073047 | | | | | |
| chr2 | 234078106 | 234078306 | 201 | 2:234078106-234078306 | | | | | |
| chr2 | 234078567 | 234078767 | 201 | 2:234078567-234078767 | | | | | |
| chr2 | 234078920 | 234079120 | 201 | 2:234078920-234079120 | | | | | |
| chr2 | 234103983 | 234104183 | 201 | 2:234103983-234104183 | | | | | |
| chr2 | 234113188 | 234113388 | 201 | 2:234113188-234113388 | | | | | |
| chr3 | 12837871 | 12838071 | 201 | 3:12837871-12838071 | | | | | |
| chr3 | 12838071 | 12838271 | 201 | 3:12838071-12838271 | | | | | |
| chr3 | 12838079 | 12838279 | 201 | 3:12838079-12838279 | | | | | |
| chr3 | 12848532 | 12848732 | 201 | 3:12848532-12848732 | | | | | |
| chr3 | 12868845 | 12869045 | 201 | 3:12868845-12869045 | | | | | |
| chr3 | 42726911 | 42727111 | 201 | 3:42726911-42727111 | | | | | |
| chr3 | 111717411 | 111717611 | 201 | 3:111717411-111717611 | | | | | |
| chr3 | 111717486 | 111717686 | 201 | 3:111717486-111717686 | | | | | |
| chr3 | 111717542 | 111717742 | 201 | 3:111717542-111717742 | | | | | |
| chr3 | 111717936 | 111718136 | 201 | 3:111717936-111718136 | | | | | |
| chr3 | 111718095 | 111718295 | 201 | 3:111718095-111718295 | | | | | |
| chr3 | 111719270 | 111719470 | 201 | 3:111719270-111719470 | | | | | |
| chr3 | 111719519 | 111719719 | 201 | 3:111719519-111719719 | | | | | |
| chr3 | 128200821 | 128201021 | 201 | 3:128200821-128201021 | | | | | |
| chr3 | 128206659 | 128206859 | 201 | 3:128206659-128206859 | | | | | |
| chr3 | 128207273 | 128207473 | 201 | 3:128207273-128207473 | | | | | |
| chr3 | 128209391 | 128209591 | 201 | 3:128209391-128209591 | | | | | |
| chr3 | 128211916 | 128212116 | 201 | 3:128211916-128212116 | | | | | |
| chr3 | 128211928 | 128212128 | 201 | 3:128211928-128212128 | | | | | |
| chr3 | 150481064 | 150481264 | 201 | 3:150481064-150481264 | | | | | |
| chr3 | 150481118 | 150481318 | 201 | 3:150481118-150481318 | | | | | |
| chr3 | 150481164 | 150481364 | 201 | 3:150481164-150481364 | | | | | |
| chr3 | 151154610 | 151154810 | 201 | 3:151154610-151154810 | | | | | |
| chr3 | 151158459 | 151158659 | 201 | 3:151158459-151158659 | | | | | |
| chr3 | 151158468 | 151158668 | 201 | 3:151158468-151158668 | | | | | |
| chr3 | 151160838 | 151161038 | 201 | 3:151160838-151161038 | | | | | |
| chr3 | 151176397 | 151176597 | 201 | 3:151176397-151176597 | | | | | |
| chr3 | 181429614 | 181429814 | 201 | 3:181429614-181429814 | | | | | |
| chr3 | 181429622 | 181429822 | 201 | 3:181429622-181429822 | | | | | |
| chr3 | 185782249 | 185782449 | 201 | 3:185782249-185782449 | | | | | |
| chr3 | 185783659 | 185783859 | 201 | 3:185783659-185783859 | | | | | |
| chr3 | 185796794 | 185796994 | 201 | 3:185796794-185796994 | | | | | |
| chr3 | 185797595 | 185797795 | 201 | 3:185797595-185797795 | | | | | |
| chr3 | 185823179 | 185823379 | 201 | 3:185823179-185823379 | | | | | |
| chr3 | 185823413 | 185823613 | 201 | 3:185823413-185823613 | | | | | |
| chr3 | 185823631 | 185823831 | 201 | 3:185823631-185823831 | | | | | |
| chr3 | 185826233 | 185826433 | 201 | 3:185826233-185826433 | | | | | |
| chr3 | 185826239 | 185826439 | 201 | 3:185826239-185826439 | | | | | |
| chr3 | 185826364 | 185826564 | 201 | 3:185826364-185826564 | | | | | |
| chr3 | 185826618 | 185826818 | 201 | 3:185826618-185826818 | | | | | |
| chr3 | 185826688 | 185826888 | 201 | 3:185826688-185826888 | | | | | |
| chr3 | 185826770 | 185826970 | 201 | 3:185826770-185826970 | | | | | |
| chr3 | 185826801 | 185827001 | 201 | 3:185826801-185827001 | | | | | |
| chr3 | 185828007 | 185828207 | 201 | 3:185828007-185828207 | | | | | |
| chr3 | 187086020 | 187086220 | 201 | 3:187086020-187086220 | | | | | |
| chr4 | 6694696 | 6694896 | 201 | 4:6694696-6694896 | | | | | |
| chr4 | 6695505 | 6695705 | 201 | 4:6695505-6695705 | | | | | |
| chr4 | 38796154 | 38796354 | 201 | 4:38796154-38796354 | | | | | |
| chr4 | 38805897 | 38806097 | 201 | 4:38805897-38806097 | | | | | |
| chr4 | 38806310 | 38806510 | 201 | 4:38806310-38806510 | | | | | |
| chr4 | 38806312 | 38806512 | 201 | 4:38806312-38806512 | | | | | |
| chr4 | 38806340 | 38806540 | 201 | 4:38806340-38806540 | | | | | |
| chr4 | 38806372 | 38806572 | 201 | 4:38806372-38806572 | | | | | |
| chr4 | 38806437 | 38806637 | 201 | 4:38806437-38806637 | | | | | |
| chr4 | 38807054 | 38807254 | 201 | 4:38807054-38807254 | | | | | |
| chr4 | 38807099 | 38807299 | 201 | 4:38807099-38807299 | | | | | |
| chr4 | 38858338 | 38858538 | 201 | 4:38858338-38858538 | | | | | |
| chr4 | 41258330 | 41258530 | 201 | 4:41258330-41258530 | | | | | |
| chr4 | 41258686 | 41258886 | 201 | 4:41258686-41258886 | | | | | |
| chr4 | 41258750 | 41258950 | 201 | 4:41258750-41258950 | | | | | |
| chr4 | 41258825 | 41259025 | 201 | 4:41258825-41259025 | | | | | |
| chr4 | 41258828 | 41259028 | 201 | 4:41258828-41259028 | | | | | |
| chr4 | 41258849 | 41259049 | 201 | 4:41258849-41259049 | | | | | |
| chr4 | 41258850 | 41259050 | 201 | 4:41258850-41259050 | | | | | |
| chr4 | 41258857 | 41259057 | 201 | 4:41258857-41259057 | | | | | |
| chr4 | 41258862 | 41259062 | 201 | 4:41258862-41259062 | | | | | |
| chr4 | 41258863 | 41259063 | 201 | 4:41258863-41259063 | | | | | |
| chr4 | 41262556 | 41262756 | 201 | 4:41262556-41262756 | | | | | |
| chr4 | 41263656 | 41263856 | 201 | 4:41263656-41263856 | | | | | |
| chr4 | 48899120 | 48899320 | 201 | 4:48899120-48899320 | | | | | |
| chr4 | 48899765 | 48899965 | 201 | 4:48899765-48899965 | | | | | |
| chr4 | 48908644 | 48908844 | 201 | 4:48908644-48908844 | | | | | |
| chr4 | 48908674 | 48908874 | 201 | 4:48908674-48908874 | | | | | |
| chr4 | 48908691 | 48908891 | 201 | 4:48908691-48908891 | | | | | |
| chr4 | 48908705 | 48908905 | 201 | 4:48908705-48908905 | | | | | |
| chr4 | 48908715 | 48908915 | 201 | 4:48908715-48908915 | | | | | |
| chr4 | 48908745 | 48908945 | 201 | 4:48908745-48908945 | | | | | |
| chr4 | 48908854 | 48909054 | 201 | 4:48908854-48909054 | | | | | |
| chr4 | 84030896 | 84031096 | 201 | 4:84030896-84031096 | | | | | |
| chr4 | 84035768 | 84035968 | 201 | 4:84035768-84035968 | | | | | |
| chr4 | 84035789 | 84035989 | 201 | 4:84035789-84035989 | | | | | |
| chr4 | 84035855 | 84036055 | 201 | 4:84035855-84036055 | | | | | |
| chr4 | 84058128 | 84058328 | 201 | 4:84058128-84058328 | | | | | |
| chr4 | 177158625 | 177158825 | 201 | 4:177158625-177158825 | | | | | |
| chr4 | 177162727 | 177162927 | 201 | 4:177162727-177162927 | | | | | |
| chr4 | 177170609 | 177170809 | 201 | 4:177170609-177170809 | | | | | |
| chr4 | 177190273 | 177190473 | 201 | 4:177190273-177190473 | | | | | |
| chr4 | 177198622 | 177198822 | 201 | 4:177198622-177198822 | | | | | |
| chr5 | 102089585 | 102089785 | 201 | 5:102089585-102089785 | | | | | |
| chr5 | 102090271 | 102090471 | 201 | 5:102090271-102090471 | | | | | |
| chr5 | 102090387 | 102090587 | 201 | 5:102090387-102090587 | | | | | |
| chr5 | 102090918 | 102091118 | 201 | 5:102090918-102091118 | | | | | |
| chr5 | 102090947 | 102091147 | 201 | 5:102090947-102091147 | | | | | |
| chr5 | 102143661 | 102143861 | 201 | 5:102143661-102143861 | | | | | |
| chr5 | 102143667 | 102143867 | 201 | 5:102143667-102143867 | | | | | |
| chr5 | 102200141 | 102200341 | 201 | 5:102200141-102200341 | | | | | |
| chr5 | 102200848 | 102201048 | 201 | 5:102200848-102201048 | | | | | |
| chr5 | 102200914 | 102201114 | 201 | 5:102200914-102201114 | | | | | |
| chr5 | 102201083 | 102201283 | 201 | 5:102201083-102201283 | | | | | |
| chr5 | 102201330 | 102201530 | 201 | 5:102201330-102201530 | | | | | |
| chr5 | 102201341 | 102201541 | 201 | 5:102201341-102201541 | | | | | |
| chr5 | 102201423 | 102201623 | 201 | 5:102201423-102201623 | | | | | |
| chr5 | 102201427 | 102201627 | 201 | 5:102201427-102201627 | | | | | |
| chr5 | 102201614 | 102201814 | 201 | 5:102201614-102201814 | | | | | |
| chr5 | 102201661 | 102201861 | 201 | 5:102201661-102201861 | | | | | |
| chr5 | 102201720 | 102201920 | 201 | 5:102201720-102201920 | | | | | |
| chr5 | 102202191 | 102202391 | 201 | 5:102202191-102202391 | | | | | |
| chr5 | 102231517 | 102231717 | 201 | 5:102231517-102231717 | | | | | |
| chr5 | 102285181 | 102285381 | 201 | 5:102285181-102285381 | | | | | |
| chr5 | 102295601 | 102295801 | 201 | 5:102295601-102295801 | | | | | |
| chr5 | 102325375 | 102325575 | 201 | 5:102325375-102325575 | | | | | |
| chr5 | 102343057 | 102343257 | 201 | 5:102343057-102343257 | | | | | |
| chr5 | 102343146 | 102343346 | 201 | 5:102343146-102343346 | | | | | |
| chr5 | 102343163 | 102343363 | 201 | 5:102343163-102343363 | | | | | |
| chr5 | 102343173 | 102343373 | 201 | 5:102343173-102343373 | | | | | |
| chr6 | 34523991 | 34524191 | 201 | 6:34523991-34524191 | | | | | |
| chr6 | 34524010 | 34524210 | 201 | 6:34524010-34524210 | | | | | |
| chr6 | 112451090 | 112451290 | 201 | 6:112451090-112451290 | | | | | |
| chr6 | 112451261 | 112451461 | 201 | 6:112451261-112451461 | | | | | |
| chr6 | 112460378 | 112460578 | 201 | 6:112460378-112460578 | | | | | |
| chr6 | 112486266 | 112486466 | 201 | 6:112486266-112486466 | | | | | |
| chr6 | 112500604 | 112500804 | 201 | 6:112500604-112500804 | | | | | |
| chr6 | 112512866 | 112513066 | 201 | 6:112512866-112513066 | | | | | |
| chr6 | 112512903 | 112513103 | 201 | 6:112512903-112513103 | | | | | |
| chr6 | 112512913 | 112513113 | 201 | 6:112512913-112513113 | | | | | |
| chr6 | 112537550 | 112537750 | 201 | 6:112537550-112537750 | | | | | |
| chr6 | 112537855 | 112538055 | 201 | 6:112537855-112538055 | | | | | |
| chr6 | 112575587 | 112575787 | 201 | 6:112575587-112575787 | | | | | |
| chr6 | 112575640 | 112575840 | 201 | 6:112575640-112575840 | | | | | |
| chr6 | 112575674 | 112575874 | 201 | 6:112575674-112575874 | | | | | |
| chr6 | 112575680 | 112575880 | 201 | 6:112575680-112575880 | | | | | |
| chr6 | 112575690 | 112575890 | 201 | 6:112575690-112575890 | | | | | |
| chr6 | 112575702 | 112575902 | 201 | 6:112575702-112575902 | | | | | |
| chr6 | 112575749 | 112575949 | 201 | 6:112575749-112575949 | | | | | |
| chr6 | 112575812 | 112576012 | 201 | 6:112575812-112576012 | | | | | |
| chr6 | 112575817 | 112576017 | 201 | 6:112575817-112576017 | | | | | |
| chr6 | 112575824 | 112576024 | 201 | 6:112575824-112576024 | | | | | |
| chr6 | 112576041 | 112576241 | 201 | 6:112576041-112576241 | | | | | |
| chr7 | 148508815 | 148509015 | 201 | 7:148508815-148509015 | | | | | |
| chr7 | 148580501 | 148580701 | 201 | 7:148580501-148580701 | | | | | |
| chr7 | 148581151 | 148581351 | 201 | 7:148581151-148581351 | | | | | |
| chr7 | 148581237 | 148581437 | 201 | 7:148581237-148581437 | | | | | |
| chr7 | 148581240 | 148581440 | 201 | 7:148581240-148581440 | | | | | |
| chr7 | 148581270 | 148581470 | 201 | 7:148581270-148581470 | | | | | |
| chr7 | 148581275 | 148581475 | 201 | 7:148581275-148581475 | | | | | |
| chr7 | 148581279 | 148581479 | 201 | 7:148581279-148581479 | | | | | |
| chr7 | 148581313 | 148581513 | 201 | 7:148581313-148581513 | | | | | |
| chr8 | 17169082 | 17169282 | 201 | 8:17169082-17169282 | | | | | |
| chr8 | 17169272 | 17169472 | 201 | 8:17169272-17169472 | | | | | |
| chr8 | 17206466 | 17206666 | 201 | 8:17206466-17206666 | | | | | |
| chr8 | 17206646 | 17206846 | 201 | 8:17206646-17206846 | | | | | |
| chr8 | 17206894 | 17207094 | 201 | 8:17206894-17207094 | | | | | |
| chr8 | 17270716 | 17270916 | 201 | 8:17270716-17270916 | | | | | |
| chr8 | 17270736 | 17270936 | 201 | 8:17270736-17270936 | | | | | |
| chr8 | 17270937 | 17271137 | 201 | 8:17270937-17271137 | | | | | |
| chr8 | 53045604 | 53045804 | 201 | 8:53045604-53045804 | | | | | |
| chr8 | 53045678 | 53045878 | 201 | 8:53045678-53045878 | | | | | |
| chr8 | 53050272 | 53050472 | 201 | 8:53050272-53050472 | | | | | |
| chr8 | 53130239 | 53130439 | 201 | 8:53130239-53130439 | | | | | |
| chr8 | 5313028 | 53130481 | 201 | 8:53130281-53130481 | | | | | |
| chr8 | 53130282 | 53130482 | 201 | 8:53130282-53130482 | | | | | |
| chr8 | 53134555 | 53134755 | 201 | 8:53134555-53134755 | | | | | |
| chr8 | 53159124 | 53159324 | 201 | 8:53159124-53159324 | | | | | |
| chr8 | 53160986 | 53161186 | 201 | 8:53160986-53161186 | | | | | |
| chr8 | 53161872 | 53162072 | 201 | 8:53161872-53162072 | | | | | |
| chr8 | 53166808 | 53167008 | 201 | 8:53166808-53167008 | | | | | |
| chr8 | 53322203 | 53322403 | 201 | 8:53322203-53322403 | | | | | |
| chr8 | 53322209 | 53322409 | 201 | 8:53322209-53322409 | | | | | |
| chr8 | 53322216 | 53322416 | 201 | 8:53322216-53322416 | | | | | |
| chr8 | 53322232 | 53322432 | 201 | 8:53322232-53322432 | | | | | |
| chr8 | 53322252 | 53322452 | 201 | 8:53322252-53322452 | | | | | |
| chr8 | 53322265 | 53322465 | 201 | 8:53322265-53322465 | | | | | |
| chr8 | 53322339 | 53322539 | 201 | 8:53322339-53322539 | | | | | |
| chr8 | 53322342 | 53322542 | 201 | 8:53322342-53322542 | | | | | |
| chr8 | 53322405 | 53322605 | 201 | 8:53322405-53322605 | | | | | |
| chr8 | 53373419 | 53373619 | 201 | 8:53373419-53373619 | | | | | |
| chr8 | 104152838 | 104153038 | 201 | 8:104152838-104153038 | | | | | |
| chr8 | 104152841 | 104153041 | 201 | 8:104152841-104153041 | | | | | |
| chr8 | 104152887 | 104153087 | 201 | 8:104152887-104153087 | | | | | |
| chr8 | 104153026 | 104153226 | 201 | 8:104153026-104153226 | | | | | |
| chr8 | 104165267 | 104165467 | 201 | 8:104165267-104165467 | | | | | |
| chr8 | 104220320 | 104220520 | 201 | 8:104220320-104220520 | | | | | |
| chr8 | 104225459 | 104225659 | 201 | 8:104225459-104225659 | | | | | |
| chr8 | 104225539 | 104225739 | 201 | 8:104225539-104225739 | | | | | |
| chr10 | 69597717 | 69597917 | 201 | 10:69597717-69597917 | | | | | |
| chr10 | 69597721 | 69597921 | 201 | 10:69597721-69597921 | | | | | |
| chr10 | 69597754 | 69597954 | 201 | 10:69597754-69597954 | | | | | |
| chr10 | 69597822 | 69598022 | 201 | 10:69597822-69598022 | | | | | |
| chr10 | 69597824 | 69598024 | 201 | 10:69597824-69598024 | | | | | |
| chr10 | 88428106 | 88428306 | 201 | 10:88428106-88428306 | | | | | |
| chr10 | 88428270 | 88428470 | 201 | 10:88428270-88428470 | | | | | |
| chr10 | 88428326 | 88428526 | 201 | 10:88428326-88428526 | | | | | |
| chr10 | 88428328 | 88428528 | 201 | 10:88428328-88428528 | | | | | |
| chr10 | 88428349 | 88428549 | 201 | 10:88428349-88428549 | | | | | |
| chr10 | 88478387 | 88478587 | 201 | 10:88478387-88478587 | | | | | |
| chr10 | 92680933 | 92681133 | 201 | 10:92680933-92681133 | | | | | |
| chr11 | 60869767 | 60869967 | 201 | 11:60869767-60869967 | | | | | |
| chr11 | 60869881 | 60870081 | 201 | 11:60869881-60870081 | | | | | |
| chr11 | 67350966 | 67351166 | 201 | 11:67350966-67351166 | | | | | |
| chr11 | 67351113 | 67351313 | 201 | 11:67351113-67351313 | | | | | |
| chr11 | 67351193 | 67351393 | 201 | 11:67351193-67351393 | | | | | |
| chr11 | 67351495 | 67351695 | 201 | 11:67351495-67351695 | | | | | |
| chr11 | 67351496 | 67351696 | 201 | 11:67351496-67351696 | | | | | |
| chr11 | 67351505 | 67351705 | 201 | 11:67351505-67351705 | | | | | |
| chr11 | 67351826 | 67352026 | 201 | 11:67351826-67352026 | | | | | |
| chr11 | 67352166 | 67352366 | 201 | 11:67352166-67352366 | | | | | |
| chr11 | 67353475 | 67353675 | 201 | 11:67353475-67353675 | | | | | |
| chr11 | 87908501 | 87908701 | 201 | 11:87908501-87908701 | | | | | |
| chr11 | 87908502 | 87908702 | 201 | 11:87908502-87908702 | | | | | |
| chr11 | 87908535 | 87908735 | 201 | 11:87908535-87908735 | | | | | |
| chr11 | 112831897 | 112832097 | 201 | 11:112831897-112832097 | | | | | |
| chr11 | 112831990 | 112832190 | 201 | 11:112831990-112832190 | | | | | |
| chr11 | 112832024 | 112832224 | 201 | 11:112832024-112832224 | | | | | |
| chr11 | 112832047 | 112832247 | 201 | 11:112832047-112832247 | | | | | |
| chr11 | 112832057 | 112832257 | 201 | 11:112832057-112832257 | | | | | |
| chr11 | 112832058 | 112832258 | 201 | 11:112832058-112832258 | | | | | |
| chr11 | 112832059 | 112832259 | 201 | 11:112832059-112832259 | | | | | |
| chr11 | 112832061 | 112832261 | 201 | 11:112832061-112832261 | | | | | |
| chr11 | 112832102 | 112832302 | 201 | 11:112832102-112832302 | | | | | |
| chr11 | 113001316 | 113001516 | 201 | 11:113001316-113001516 | | | | | |
| chr11 | 113003550 | 113003750 | 201 | 11:113003550-113003750 | | | | | |
| chr11 | 113067815 | 113068015 | 201 | 11:113067815-113068015 | | | | | |
| chr11 | 113072979 | 113073179 | 201 | 11:113072979-113073179 | | | | | |
| chr11 | 113072998 | 113073198 | 201 | 11:113072998-113073198 | | | | | |
| chr11 | 113102002 | 113102202 | 201 | 11:113102002-113102202 | | | | | |
| chr11 | 113102884 | 113103084 | 201 | 11:113102884-113103084 | | | | | |
| chr11 | 113102947 | 113103147 | 201 | 11:113102947-113103147 | | | | | |
| chr11 | 113103415 | 113103615 | 201 | 11:113103415-113103615 | | | | | |
| chr11 | 113103417 | 113103617 | 201 | 11:113103417-113103617 | | | | | |
| chr11 | 113103766 | 113103966 | 201 | 11:113103766-113103966 | | | | | |
| chr11 | 113103880 | 113104080 | 201 | 11:113103880-113104080 | | | | | |
| chr11 | 113111129 | 113111329 | 201 | 11:113111129-113111329 | | | | | |
| chr11 | 113116730 | 113116930 | 201 | 11:113116730-113116930 | | | | | |
| chr11 | 113126572 | 113126772 | 201 | 11:113126572-113126772 | | | | | |
| chr11 | 113130713 | 113130913 | 201 | 11:113130713-113130913 | | | | | |
| chr11 | 113130880 | 113131080 | 201 | 11:113130880-113131080 | | | | | |
| chr11 | 113140591 | 113140791 | 201 | 11:113140591-113140791 | | | | | |
| chr11 | 113143642 | 113143842 | 201 | 11:113143642-113143842 | | | | | |
| chr11 | 133938720 | 133938920 | 201 | 11:133938720-133938920 | | | | | |
| chr11 | 133938866 | 133939066 | 201 | 11:133938866-133939066 | | | | | |
| chr11 | 133938880 | 133939080 | 201 | 11:133938880-133939080 | | | | | |
| chr11 | 133996134 | 133996334 | 201 | 11:133996134-133996334 | | | | | |
| chr11 | 134009435 | 134009635 | 201 | 11:134009435-134009635 | | | | | |
| chr11 | 134017593 | 134017793 | 201 | 11:134017593-134017793 | | | | | |
| chr12 | 7022809 | 7023009 | 201 | 12:7022809-7023009 | | | | | |
| chr12 | 7023391 | 7023591 | 201 | 12:7023391-7023591 | | | | | |
| chr12 | 7023514 | 7023714 | 201 | 12:7023514-7023714 | | | | | |
| chr12 | 7023602 | 7023802 | 201 | 12:7023602-7023802 | | | | | |
| chr12 | 7023621 | 7023821 | 201 | 12:7023621-7023821 | | | | | |
| chr12 | 7023653 | 7023853 | 201 | 12:7023653-7023853 | | | | | |
| chr12 | 7023658 | 7023858 | 201 | 12:7023658-7023858 | | | | | |
| chr12 | 7023661 | 7023861 | 201 | 12:7023661-7023861 | | | | | |
| chr12 | 7023674 | 7023874 | 201 | 12:7023674-7023874 | | | | | |
| chr12 | 7024271 | 7024471 | 201 | 12:7024271-7024471 | | | | | |
| chr12 | 7026393 | 7026593 | 201 | 12:7026393-7026593 | | | | | |
| chr12 | 7028638 | 7028838 | 201 | 12:7028638-7028838 | | | | | |
| chr12 | 7030644 | 7030844 | 201 | 12:7030644-7030844 | | | | | |
| chr12 | 7031052 | 7031252 | 201 | 12:7031052-7031252 | | | | | |
| chr12 | 24983162 | 24983362 | 201 | 12:24983162-24983362 | | | | | |
| chr12 | 25055130 | 25055330 | 201 | 12:25055130-25055330 | | | | | |
| chr12 | 25055222 | 25055422 | 201 | 12:25055222-25055422 | | | | | |
| chr12 | 25055909 | 25056109 | 201 | 12:25055909-25056109 | | | | | |
| chr12 | 25101877 | 25102077 | 201 | 12:25101877-25102077 | | | | | |
| chr12 | 25101883 | 25102083 | 201 | 12:25101883-25102083 | | | | | |
| chr12 | 25101895 | 25102095 | 201 | 12:25101895-25102095 | | | | | |
| chr12 | 25101920 | 25102120 | 201 | 12:25101920-25102120 | | | | | |
| chr12 | 25102293 | 25102493 | 201 | 12:25102293-25102493 | | | | | |
| chr12 | 25173836 | 25174036 | 201 | 12:25173836-25174036 | | | | | |
| chr12 | 25186593 | 25186793 | 201 | 12:25186593-25186793 | | | | | |
| chr12 | 25188314 | 25188514 | 201 | 12:25188314-25188514 | | | | | |
| chr12 | 25205089 | 25205289 | 201 | 12:25205089-25205289 | | | | | |
| chr12 | 25205105 | 25205305 | 201 | 12:25205105-25205305 | | | | | |
| chr12 | 25205106 | 25205306 | 201 | 12:25205106-25205306 | | | | | |
| chr12 | 25205346 | 25205546 | 201 | 12:25205346-25205546 | | | | | |
| chr12 | 25205421 | 25205621 | 201 | 12:25205421-25205621 | | | | | |
| chr12 | 25205476 | 25205676 | 201 | 12:25205476-25205676 | | | | | |
| chr12 | 25205499 | 25205699 | 201 | 12:25205499-25205699 | | | | | |
| chr12 | 25205534 | 25205734 | 201 | 12:25205534-25205734 | | | | | |
| chr12 | 25205574 | 25205774 | 201 | 12:25205574-25205774 | | | | | |
| chr12 | 25205779 | 25205979 | 201 | 12:25205779-25205979 | | | | | |
| chr12 | 25228574 | 25228774 | 201 | 12:25228574-25228774 | | | | | |
| chr12 | 25242608 | 25242808 | 201 | 12:25242608-25242808 | | | | | |
| chr12 | 25249767 | 25249967 | 201 | 12:25249767-25249967 | | | | | |
| chr12 | 25249792 | 25249992 | 201 | 12:25249792-25249992 | | | | | |
| chr12 | 25257163 | 25257363 | 201 | 12:25257163-25257363 | | | | | |
| chr12 | 66582559 | 66582759 | 201 | 12:66582559-66582759 | | | | | |
| chr12 | 66582875 | 66583075 | 201 | 12:66582875-66583075 | | | | | |
| chr12 | 66582937 | 66583137 | 201 | 12:66582937-66583137 | | | | | |
| chr12 | 70082891 | 70083091 | 201 | 12:70082891-70083091 | | | | | |
| chr12 | 70082956 | 70083156 | 201 | 12:70082956-70083156 | | | | | |
| chr12 | 70087752 | 70087952 | 201 | 12:70087752-70087952 | | | | | |
| chr12 | 70092965 | 70093165 | 201 | 12:70092965-70093165 | | | | | |
| chr12 | 70093015 | 70093215 | 201 | 12:70093015-70093215 | | | | | |
| chr12 | 70093024 | 70093224 | 201 | 12:70093024-70093224 | | | | | |
| chr12 | 70093031 | 70093231 | 201 | 12:70093031-70093231 | | | | | |
| chr12 | 70093041 | 70093241 | 201 | 12:70093041-70093241 | | | | | |
| chr12 | 70093044 | 70093244 | 201 | 12:70093044-70093244 | | | | | |
| chr12 | 70093075 | 70093275 | 201 | 12:70093075-70093275 | | | | | |
| chr12 | 70093096 | 70093296 | 201 | 12:70093096-70093296 | | | | | |
| chr12 | 70093156 | 70093356 | 201 | 12:70093156-70093356 | | | | | |
| chr12 | 103351364 | 103351564 | 201 | 12:103351364-103351564 | | | | | |
| chr13 | 102527544 | 102527744 | 201 | 13:102527544-102527744 | | | | | |
| chr13 | 102568895 | 102569095 | 201 | 13:102568895-102569095 | | | | | |
| chr13 | 103054024 | 103054224 | 201 | 13:103054024-103054224 | | | | | |
| chr14 | 21994247 | 21994447 | 201 | 14:21994247-21994447 | | | | | |
| chr14 | 21994261 | 21994461 | 201 | 14:21994261-21994461 | | | | | |
| chr14 | 21994534 | 21994734 | 201 | 14:21994534-21994734 | | | | | |
| chr14 | 22004936 | 22005136 | 201 | 14:22004936-22005136 | | | | | |
| chr14 | 22004968 | 22005168 | 201 | 14:22004968-22005168 | | | | | |
| chr14 | 22005138 | 22005338 | 201 | 14:22005138-22005338 | | | | | |
| chr14 | 22005250 | 22005450 | 201 | 14:22005250-22005450 | | | | | |
| chr14 | 51448929 | 51449129 | 201 | 14:51448929-51449129 | | | | | |
| chr14 | 51453070 | 51453270 | 201 | 14:51453070-51453270 | | | | | |
| chr14 | 51454994 | 51455194 | 201 | 14:51454994-51455194 | | | | | |
| chr14 | 51561694 | 51561894 | 201 | 14:51561694-51561894 | | | | | |
| chr14 | 51561947 | 51562147 | 201 | 14:51561947-51562147 | | | | | |
| chr14 | 51562679 | 51562879 | 201 | 14:51562679-51562879 | | | | | |
| chr14 | 75988668 | 75988868 | 201 | 14:75988668-75988868 | | | | | |
| chr14 | 75988698 | 75988898 | 201 | 14:75988698-75988898 | | | | | |
| chr14 | 75988803 | 75989003 | 201 | 14:75988803-75989003 | | | | | |
| chr14 | 93389325 | 93389525 | 201 | 14:93389325-93389525 | | | | | |
| chr14 | 93389402 | 93389602 | 201 | 14:93389402-93389602 | | | | | |
| chr14 | 93389421 | 93389621 | 201 | 14:93389421-93389621 | | | | | |
| chr14 | 93389608 | 93389808 | 201 | 14:93389608-93389808 | | | | | |
| chr14 | 93392346 | 93392546 | 201 | 14:93392346-93392546 | | | | | |
| chr14 | 93397922 | 93398122 | 201 | 14:93397922-93398122 | | | | | |
| chr15 | 74465912 | 74466112 | 201 | 15:74465912-74466112 | | | | | |
| chr15 | 74466371 | 74466571 | 201 | 15:74466371-74466571 | | | | | |
| chr15 | 74466644 | 74466844 | 201 | 15:74466644-74466844 | | | | | |
| chr15 | 74466742 | 74466942 | 201 | 15:74466742-74466942 | | | | | |
| chr15 | 77285600 | 77285800 | 201 | 15:77285600-77285800 | | | | | |
| chr15 | 77286921 | 77287121 | 201 | 15:77286921-77287121 | | | | | |
| chr15 | 77287326 | 77287526 | 201 | 15:77287326-77287526 | | | | | |
| chr15 | 77287363 | 77287563 | 201 | 15:77287363-77287563 | | | | | |
| chr15 | 77287365 | 77287565 | 201 | 15:77287365-77287565 | | | | | |
| chr15 | 77287367 | 77287567 | 201 | 15:77287367-77287567 | | | | | |
| chr15 | 77287372 | 77287572 | 201 | 15:77287372-77287572 | | | | | |
| chr15 | 77287413 | 77287613 | 201 | 15:77287413-77287613 | | | | | |
| chr15 | 77287603 | 77287803 | 201 | 15:77287603-77287803 | | | | | |
| chr15 | 77287626 | 77287826 | 201 | 15:77287626-77287826 | | | | | |
| chr15 | 77287653 | 77287853 | 201 | 15:77287653-77287853 | | | | | |
| chr15 | 77287660 | 77287860 | 201 | 15:77287660-77287860 | | | | | |
| chr15 | 77287815 | 77288015 | 201 | 15:77287815-77288015 | | | | | |
| chr15 | 77308052 | 77308252 | 201 | 15:77308052-77308252 | | | | | |
| chr15 | 77310699 | 77310899 | 201 | 15:77310699-77310899 | | | | | |
| chr15 | 77319966 | 77320166 | 201 | 15:77319966-77320166 | | | | | |
| chr15 | 77320584 | 77320784 | 201 | 15:77320584-77320784 | | | | | |
| chr15 | 77324558 | 77324758 | 201 | 15:77324558-77324758 | | | | | |
| chr15 | 77327935 | 77328135 | 201 | 15:77327935-77328135 | | | | | |
| chr16 | 29674500 | 29674700 | 201 | 16:29674500-29674700 | | | | | |
| chr16 | 29690229 | 29690429 | 201 | 16:29690229-29690429 | | | | | |
| chr16 | 29690258 | 29690458 | 201 | 16:29690258-29690458 | | | | | |
| chr16 | 29690403 | 29690603 | 201 | 16:29690403-29690603 | | | | | |
| chr16 | 29707018 | 29707218 | 201 | 16:29707018-29707218 | | | | | |
| chr16 | 31146861 | 31147061 | 201 | 16:31146861-31147061 | | | | | |
| chr16 | 31146924 | 31147124 | 201 | 16:31146924-31147124 | | | | | |
| chr16 | 31146948 | 31147148 | 201 | 16:31146948-31147148 | | | | | |
| chr16 | 31146983 | 31147183 | 201 | 16:31146983-31147183 | | | | | |
| chr16 | 74710849 | 74711049 | 201 | 16:74710849-74711049 | | | | | |
| chr16 | 74712211 | 74712411 | 201 | 16:74712211-74712411 | | | | | |
| chr16 | 74712746 | 74712946 | 201 | 16:74712746-74712946 | | | | | |
| chr16 | 74725267 | 74725467 | 201 | 16:74725267-74725467 | | | | | |
| chr16 | 74734588 | 74734788 | 201 | 16:74734588-74734788 | | | | | |
| chr16 | 74734607 | 74734807 | 201 | 16:74734607-74734807 | | | | | |
| chr16 | 74734712 | 74734912 | 201 | 16:74734712-74734912 | | | | | |
| chr16 | 74734758 | 74734958 | 201 | 16:74734758-74734958 | | | | | |
| chr16 | 88713133 | 88713333 | 201 | 16:88713133-88713333 | | | | | |
| chr16 | 88716622 | 88716822 | 201 | 16:88716622-88716822 | | | | | |
| chr16 | 88717296 | 88717496 | 201 | 16:88717296-88717496 | | | | | |
| chr16 | 88717323 | 88717523 | 201 | 16:88717323-88717523 | | | | | |
| chr16 | 88717338 | 88717538 | 201 | 16:88717338-88717538 | | | | | |
| chr16 | 88717339 | 88717539 | 201 | 16:88717339-88717539 | | | | | |
| chr16 | 88717343 | 88717543 | 201 | 16:88717343-88717543 | | | | | |
| chr16 | 88717349 | 88717549 | 201 | 16:88717349-88717549 | | | | | |
| chr16 | 88717362 | 88717562 | 201 | 16:88717362-88717562 | | | | | |
| chr16 | 88717460 | 88717660 | 201 | 16:88717460-88717660 | | | | | |
| chr17 | 1665153 | 1665353 | 201 | 17:1665153-1665353 | | | | | |
| chr17 | 1665223 | 1665423 | 201 | 17:1665223-1665423 | | | | | |
| chr17 | 1665255 | 1665455 | 201 | 17:1665255-1665455 | | | | | |
| chr17 | 1665262 | 1665462 | 201 | 17:1665262-1665462 | | | | | |
| chr17 | 1665306 | 1665506 | 201 | 17:1665306-1665506 | | | | | |
| chr17 | 1666023 | 1666223 | 201 | 17:1666023-1666223 | | | | | |
| chr17 | 1669468 | 1669668 | 201 | 17:1669468-1669668 | | | | | |
| chr17 | 1673720 | 1673920 | 201 | 17:1673720-1673920 | | | | | |
| chr17 | 1674913 | 1675113 | 201 | 17:1674913-1675113 | | | | | |
| chr17 | 1679644 | 1679844 | 201 | 17:1679644-1679844 | | | | | |
| chr17 | 27283357 | 27283557 | 201 | 17:27283357-27283557 | | | | | |
| chr17 | 27284731 | 27284931 | 201 | 17:27284731-27284931 | | | | | |
| chr17 | 27287891 | 27288091 | 201 | 17:27287891-27288091 | | | | | |
| chr17 | 27308510 | 27308710 | 201 | 17:27308510-27308710 | | | | | |
| chr17 | 27308790 | 27308990 | 201 | 17:27308790-27308990 | | | | | |
| chr17 | 27332831 | 27333031 | 201 | 17:27332831-27333031 | | | | | |
| chr17 | 27332981 | 27333181 | 201 | 17:27332981-27333181 | | | | | |
| chr17 | 27333063 | 27333263 | 201 | 17:27333063-27333263 | | | | | |
| chr17 | 27333216 | 27333416 | 201 | 17:27333216-27333416 | | | | | |
| chr17 | 27333358 | 27333558 | 201 | 17:27333358-27333558 | | | | | |
| chr17 | 62007774 | 62007974 | 201 | 17:62007774-62007974 | | | | | |
| chr17 | 62009521 | 62009721 | 201 | 17:62009521-62009721 | | | | | |
| chr17 | 62009583 | 62009783 | 201 | 17:62009583-62009783 | | | | | |
| chr17 | 62009591 | 62009791 | 201 | 17:62009591-62009791 | | | | | |
| chr17 | 62009596 | 62009796 | 201 | 17:62009596-62009796 | | | | | |
| chr17 | 62009614 | 62009814 | 201 | 17:62009614-62009814 | | | | | |
| chr17 | 76970778 | 76970978 | 201 | 17:76970778-76970978 | | | | | |
| chr17 | 76970979 | 76971179 | 201 | 17:76970979-76971179 | | | | | |
| chr17 | 76972138 | 76972338 | 201 | 17:76972138-76972338 | | | | | |
| chr17 | 76973198 | 76973398 | 201 | 17:76973198-76973398 | | | | | |
| chr17 | 76973305 | 76973505 | 201 | 17:76973305-76973505 | | | | | |
| chr17 | 76975825 | 76976025 | 201 | 17:76975825-76976025 | | | | | |
| chr17 | 76975857 | 76976057 | 201 | 17:76975857-76976057 | | | | | |
| chr17 | 76975873 | 76976073 | 201 | 17:76975873-76976073 | | | | | |
| chr17 | 76975890 | 76976090 | 201 | 17:76975890-76976090 | | | | | |
| chr17 | 76975893 | 76976093 | 201 | 17:76975893-76976093 | | | | | |
| chr17 | 76975900 | 76976100 | 201 | 17:76975900-76976100 | | | | | |
| chr17 | 76975905 | 76976105 | 201 | 17:76975905-76976105 | | | | | |
| chr17 | 76975909 | 76976109 | 201 | 17:76975909-76976109 | | | | | |
| chr17 | 76975917 | 76976117 | 201 | 17:76975917-76976117 | | | | | |
| chr17 | 76975929 | 76976129 | 201 | 17:76975929-76976129 | | | | | |
| chr17 | 76976091 | 76976291 | 201 | 17:76976091-76976291 | | | | | |
| chr18 | 25616449 | 25616649 | 201 | 18:25616449-25616649 | | | | | |
| chr18 | 25625460 | 25625660 | 201 | 18:25625460-25625660 | | | | | |
| chr18 | 25677093 | 25677293 | 201 | 18:25677093-25677293 | | | | | |
| chr18 | 25739161 | 25739361 | 201 | 18:25739161-25739361 | | | | | |
| chr18 | 25757310 | 25757510 | 201 | 18:25757310-25757510 | | | | | |
| chr18 | 31158479 | 31158679 | 201 | 18:31158479-31158679 | | | | | |
| chr18 | 31158496 | 31158696 | 201 | 18:31158496-31158696 | | | | | |
| chr18 | 31158505 | 31158705 | 201 | 18:31158505-31158705 | | | | | |
| chr18 | 31158508 | 31158708 | 201 | 18:31158508-31158708 | | | | | |
| chr18 | 31158530 | 31158730 | 201 | 18:31158530-31158730 | | | | | |
| chr18 | 31158635 | 31158835 | 201 | 18:31158635-31158835 | | | | | |
| chr18 | 31158746 | 31158946 | 201 | 18:31158746-31158946 | | | | | |
| chr18 | 31185450 | 31185650 | 201 | 18:31185450-31185650 | | | | | |
| chr18 | 31185451 | 31185651 | 201 | 18:31185451-31185651 | | | | | |
| chr18 | 31185458 | 31185658 | 201 | 18:31185458-31185658 | | | | | |
| chr18 | 31185463 | 31185663 | 201 | 18:31185463-31185663 | | | | | |
| chr18 | 31185472 | 31185672 | 201 | 18:31185472-31185672 | | | | | |
| chr18 | 31185490 | 31185690 | 201 | 18:31185490-31185690 | | | | | |
| chr18 | 31187471 | 31187671 | 201 | 18:31187471-31187671 | | | | | |
| chr19 | 39989435 | 39989635 | 201 | 19:39989435-39989635 | | | | | |
| chr19 | 39989507 | 39989707 | 201 | 19:39989507-39989707 | | | | | |
| chr19 | 39989508 | 39989708 | 201 | 19:39989508-39989708 | | | | | |
| chr19 | 46916741 | 46916941 | 201 | 19:46916741-46916941 | | | | | |
| chr19 | 51358071 | 51358271 | 201 | 19:51358071-51358271 | | | | | |
| chr19 | 51358077 | 51358277 | 201 | 19:51358077-51358277 | | | | | |
| chr19 | 51358086 | 51358286 | 201 | 19:51358086-51358286 | | | | | |
| chr19 | 51358091 | 51358291 | 201 | 19:51358091-51358291 | | | | | |
| chr19 | 51358105 | 51358305 | 201 | 19:51358105-51358305 | | | | | |
| chr19 | 51358112 | 51358312 | 201 | 19:51358112-51358312 | | | | | |
| chr19 | 51358128 | 51358328 | 201 | 19:51358128-51358328 | | | | | |
| chr19 | 51360547 | 51360747 | 201 | 19:51360547-51360747 | | | | | |
| chr20 | 3210322 | 3210522 | 201 | 20:3210322-3210522 | | | | | |
| chr20 | 3211106 | 3211306 | 201 | 20:3211106-3211306 | | | | | |
| chr20 | 3218273 | 3218473 | 201 | 20:3218273-3218473 | | | | | |
| chr20 | 3218712 | 3218912 | 201 | 20:3218712-3218912 | | | | | |
| chr20 | 3219259 | 3219459 | 201 | 20:3219259-3219459 | | | | | |
| chr20 | 3219736 | 3219936 | 201 | 20:3219736-3219936 | | | | | |
| chr20 | 5891976 | 5892176 | 201 | 20:5891976-5892176 | | | | | |
| chr20 | 5892076 | 5892276 | 201 | 20:5892076-5892276 | | | | | |
| chr20 | 5896044 | 5896244 | 201 | 20:5896044-5896244 | | | | | |
| chr20 | 20348665 | 20348865 | 201 | 20:20348665-20348865 | | | | | |
| chr20 | 22565001 | 22565201 | 201 | 20:22565001-22565201 | | | | | |
| chr20 | 22565993 | 22566193 | 201 | 20:22565993-22566193 | | | | | |
| chr21 | 42839929 | 42840129 | 201 | 21:42839929-42840129 | | | | | |
| chr21 | 42840664 | 42840864 | 201 | 21:42840664-42840864 | | | | | |
| chr21 | 42878431 | 42878631 | 201 | 21:42878431-42878631 | | | | | |
| chr21 | 42879809 | 42880009 | 201 | 21:42879809-42880009 | | | | | |
| chr21 | 42879892 | 42880092 | 201 | 21:42879892-42880092 | | | | | |
| chr21 | 42879919 | 42880119 | 201 | 21:42879919-42880119 | | | | | |
| chr21 | 42879941 | 42880141 | 201 | 21:42879941-42880141 | | | | | |
| chr21 | 42879985 | 42880185 | 201 | 21:42879985-42880185 | | | | | |
| chr21 | 42879986 | 42880186 | 201 | 21:42879986-42880186 | | | | | |
| chr21 | 42902925 | 42903125 | 201 | 21:42902925-42903125 | | | | | |
| chr21 | 42902943 | 42903143 | 201 | 21:42902943-42903143 | | | | | |
| chrX | 49056535 | 49056735 | 201 | X:49056535-49056735 | | | | | |
| chrX | 49056552 | 49056752 | 201 | X:49056552-49056752 | | | | | |
| chrX | 49056554 | 49056754 | 201 | X:49056554-49056754 | | | | | |
| chrX | 49056558 | 49056758 | 201 | X:49056558-49056758 | | | | | |
| chrX | 49056618 | 49056818 | 201 | X:49056618-49056818 | | | | | |
| chrX | 66764365 | 66764565 | 201 | X:66764365-66764565 | | | | | |
| chrX | 66764562 | 66764762 | 201 | X:66764562-66764762 | | | | | |
| chrX | 66764886 | 66765086 | 201 | X:66764886-66765086 | | | | | |
| chrX | 66788631 | 66788831 | 201 | X:66788631-66788831 | | | | | |
| chr1 | 202039153 | 202039152 | 0 | 1:202039153-202039152 | | | | | |
| chr1 | 241206098 | 241206097 | 0 | 1:241206098-241206097 | | | | | |
| chr2 | 89530057 | 89530056 | 0 | 2:89530057-89530056 | | | | | |
| chr3 | 193490472 | 193490471 | 0 | 3:193490472-193490471 | | | | | |
| chr5 | 1548487 | 1548486 | 0 | 5:1548487-1548486 | | | | | |
| chr5 | 36521979 | 36521978 | 0 | 5:36521979-36521978 | | | | | |
| chr5 | 39390555 | 39390554 | 0 | 5:39390555-39390554 | | | | | |
| chr5 | 71146894 | 71146893 | 0 | 5:71146894-71146893 | | | | | |
| chr5 | 142194123 | 142194122 | 0 | 5:142194123-142194122 | | | | | |
| chr5 | 168573593 | 168573592 | 0 | 5:168573593-168573592 | | | | | |
| chr7 | 2006579 | 2006578 | 0 | 7:2006579-2006578 | | | | | |
| chr7 | 7799018 | 7799017 | 0 | 7:7799018-7799017 | | | | | |
| chr7 | 127841628 | 127841627 | 0 | 7:127841628-127841627 | | | | | |
| chr7 | 129912398 | 129912397 | 0 | 7:129912398-129912397 | | | | | |
| chr8 | 53499560 | 53499559 | 0 | 8:53499560-53499559 | | | | | |
| chr8 | 61990390 | 61990389 | 0 | 8:61990390-61990389 | | | | | |
| chr8 | 69087227 | 69087226 | 0 | 8:69087227-69087226 | | | | | |
| chr8 | 73479990 | 73479989 | 0 | 8:73479990-73479989 | | | | | |
| chr8 | 73849054 | 73849053 | 0 | 8:73849054-73849053 | | | | | |
| chr8 | 133070961 | 133070960 | 0 | 8:133070961-133070960 | | | | | |
| chr8 | 140727998 | 140727997 | 0 | 8:140727998-140727997 | | | | | |
| chr8 | 141458926 | 141458925 | 0 | 8:141458926-141458925 | | | | | |
| chr8 | 141994084 | 141994083 | 0 | 8:141994084-141994083 | | | | | |
| chr8 | 142367954 | 142367953 | 0 | 8:142367954-142367953 | | | | | |
| chr8 | 143395944 | 143395943 | 0 | 8:143395944-143395943 | | | | | |
| chr9 | 36748433 | 36748432 | 0 | 9:36748433-36748432 | | | | | |
| chr9 | 38437314 | 38437313 | 0 | 9:38437314-38437313 | | | | | |
| chr9 | 93792388 | 93792387 | 0 | 9:93792388-93792387 | | | | | |
| chr9 | 133769512 | 133769511 | 0 | 9:133769512-133769511 | | | | | |
| chr9 | 134913164 | 134913163 | 0 | 9:134913164-134913163 | | | | | |
| chr9 | 138719304 | 138719303 | 0 | 9:138719304-138719303 | | | | | |
| chr14 | 100039425 | 100039424 | 0 | 14:100039425-100039424 | | | | | |
| chr18 | 8660098 | 8660097 | 0 | 18:8660098-8660097 | | | | | |
| chr18 | 14917016 | 14917015 | 0 | 18:14917016-14917015 | | | | | |
| chr18 | 25442246 | 25442245 | 0 | 18:25442246-25442245 | | | | | |
| chr18 | 47258089 | 47258088 | 0 | 18:47258089-47258088 | | | | | |
| chr20 | 20421462 | 20421461 | 0 | 20:20421462-20421461 | | | | | |
| chr20 | 21563921 | 21563920 | 0 | 20:21563921-21563920 | | | | | |
| chr21 | 32546326 | 32546325 | 0 | 21:32546326-32546325 | | | | | |
| chr21 | 46885392 | 46885391 | 0 | 21:46885392-46885391 | | | | | |

For the “region_set” and “selection_set” columns, note that “rockermeth” denotes “rockermeth_NEvsAdeno”; “rockermethSegs” denotes “cellDMC_rockermethSegs”;
“DMS_GOI” denotes “DMS_genesOfInterest”; “AUC_Δβ_GOI” denotes “AUC_deltaBeta_GenesOfInterest”; “AUC_Δβ_TS” denotes “AUC_deltaBeta_TopSignal”;
“P_DEgenes” denotes “Promoter_DEgenes”; “ExpMeth” denotes “ExpMeth_DE_in_PARCB_NE_or_GeneOfInterest”; and “CpG_2020” denotes
“CpG_site_DNAmeth_classifierJCI_2020”.

| | | | | meth | | Peak | | |
|---|---|---|---|---|---|---|---|---|
| | start_hg19 | region_set | selection_test | state | priority_in_set | ID | f_anno | extra_anno |
| chr1 | 32237823 | mCRPC_PBMC | limma_eBayes | 1 | 32 | 1 | Intergenic | |
| chr1 | 43814069 | mCRPC_PBMC | limma_eBayes | −1 | 23 | 2 | intron | (NM_005373, |
| chr1 | 151345023 | mCRPC_PBMC | limma_eBayes | −1 | 2 | 3 | promoter-TSS | (NM_001258288) |
| chr1 | 151810787 | mCRPC_PBMC | limma_eBayes | 1 | 3 | 4 | non-coding | (NR_024237, |
| chr2 | 19561227 | mCRPC_PBMC | limma_eBayes | 1 | 18 | 5 | Intergenic | |
| chr2 | 63283706 | mCRPC_PBMC | limma_eBayes | 1 | 26 | 6 | non-coding | (NR_130153, |
| chr2 | 177030050 | mCRPC_PBMC | limma_eBayes | 1 | 24 | 7 | intron | (NM_006898, |
| chr2 | 201450223 | mCRPC_PBMC | limma_eBayes | 1 | 23 | 8 | promoter-TSS | (NM_001159) |
| chr3 | 50378091 | mCRPC_PBMC | limma_eBayes | 1 | 11 | 9 | promoter-TSS | (NM_170714) |
| chr3 | 122296269 | mCRPC_PBMC | limma_eBayes | 1 | 14 | 10 | promoter-TSS | (NM_001113523) |
| chr3 | 124860516 | mCRPC_PBMC | limma_eBayes | 1 | 8 | 11 | intron | (NM_024628, |
| chr5 | 958708 | mCRPC_PBMC | limma_eBayes | −1 | 22 | 12 | Intergenic | |
| chr5 | 3187966 | mCRPC_PBMC | limma_eBayes | −1 | 16 | 13 | Intergenic | |
| chr5 | 180230667 | mCRPC_PBMC | limma_eBayes | 1 | 20 | 14 | promoter-TSS | (NM_001364390) |
| chr6 | 13014771 | mCRPC_PBMC | limma_eBayes | −1 | 27 | 15 | intron | (NM_001322314, |
| chr6 | 15505245 | mCRPC_PBMC | limma_eBayes | −1 | 1 | 16 | intron | (NM_004973, |
| chr6 | 26271093 | mCRPC_PBMC | limma_eBayes | 1 | 12 | 17 | exon | (NM_003534, |
| chr7 | 73245339 | mCRPC_PBMC | limma_eBayes | −1 | 3 | 18 | exon | (NM_001305, |
| chr7 | 129425230 | mCRPC_PBMC | limma_eBayes | 1 | 29 | 19 | Intergenic | |
| chr8 | 56852048 | mCRPC_PBMC | limma_eBayes | 1 | 13 | 20 | intron | (NM_002350, |
| chr8 | 99960398 | mCRPC_PBMC | limma_eBayes | 1 | 10 | 21 | exon | (NM_001286841, |
| chr8 | 143625497 | mCRPC_PBMC | limma_eBayes | −1 | 29 | 22 | 3′ | UTR |
| chr8 | 145104006 | mCRPC_PBMC | limma_eBayes | 1 | 2 | 23 | promoter-TSS | (NR_158554) |
| chr10 | 6297648 | mCRPC_PBMC | limma_eBayes | −1 | 21 | 24 | Intergenic | |
| chr10 | 22541895 | mCRPC_PBMC | limma_eBayes | 1 | 16 | 25 | non-coding | (NR_038921, |
| chr11 | 393838 | mCRPC_PBMC | limma_eBayes | −1 | 26 | 26 | promoter-TSS | (NM_007183) |
| chr11 | 47416258 | mCRPC_PBMC | limma_eBayes | 1 | 22 | 27 | Intergenic | |
| chr11 | 63974672 | mCRPC_PBMC | limma_eBayes | 1 | 21 | 28 | exon | (NM_031471, |
| chr11 | 165408329 | mCRPC_PBMC | limma_eBayes | 1 | 30 | 29 | exon | (NM_153253, |
| chr11 | 67204996 | mCRPC_PBMC | limma_eBayes | 1 | 5 | 30 | promoter-TSS | (NM_005608) |
| chr11 | 69469526 | mCRPC_PBMC | limma_eBayes | −1 | 6 | 31 | TTS | (NM_053056) |
| chr12 | 7060087 | mCRPC_PBMC | limma_eBayes | 1 | 25 | 32 | promoter-TSS | (NM_080549) |
| chr12 | 53298578 | mCRPC_PBMC | limma_eBayes | −1 | 17 | 33 | promoter-TSS | (NM_002273) |
| chr12 | 95941769 | mCRPC_PBMC | limma_eBayes | 1 | 19 | 34 | promoter-TSS | (NM_001278393) |
| chr12 | 107038917 | mCRPC_PBMC | limma_eBayes | −1 | 13 | 35 | intron | (NM_213594, |
| chr12 | 124773568 | mCRPC_PBMC | limma_eBayes | −1 | 28 | 36 | 5′ | UTR |
| chr12 | 124950569 | mCRPC_PBMC | limma_eBayes | 1 | 27 | 37 | exon | (NM_001077261, |
| chr16 | 1128589 | mCRPC_PBMC | limma_eBayes | −1 | 31 | 38 | promoter-TSS | (NM_001053) |
| chr17 | 26732020 | mCRPC_PBMC | limma_eBayes | −1 | 32 | 39 | exon | (NM_001242366, |
| chr17 | 41363402 | mCRPC_PBMC | limma_eBayes | 1 | 17 | 40 | promoter-TSS | (NM_001291586) |
| chr17 | 43318510 | mCRPC_PBMC | limma_eBayes | −1 | 7 | 41 | exon | (NM_005892, |
| chr17 | 79251155 | mCRPC_PBMC | limma_eBayes | −1 | 8 | 42 | intron | (NM_001037984, |
| chr17 | 80358729 | mCRPC_PBMC | limma_eBayes | 1 | 4 | 43 | intron | (NM_175902, |
| chr17 | 180847037 | mCRPC_PBMC | limma_eBayes | −1 | 10 | 44 | intron | (NM_005993, |
| chr19 | 2085186 | mCRPC_PBMC | limma_eBayes | 1 | 31 | 45 | intron | (NM_130807, |
| chr19 | 2588141 | mCRPC_PBMC | limma_eBayes | −1 | 14 | 46 | intron | (NM_052847, |
| chr19 | 3179264 | mCRPC_PBMC | limma_eBayes | 1 | 6 | 47 | exon | (NM_003775, |
| chr19 | 10735906 | mCRPC_PBMC | limma_eBayes | −1 | 12 | 48 | promoter-TSS | (NM_001363611) |
| chr19 | 15568260 | mCRPC_PBMC | limma_eBayes | −1 | 5 | 49 | exon | (NM_001348028, |
| chr19 | 18872937 | mCRPC_PBMC | limma_eBayes | −1 | 25 | 50 | intron | (NM_001098482, |
| chr19 | 35940325 | mCRPC_PBMC | limma_eBayes | −1 | 15 | 51 | intron | (NM_005306, |
| chr19 | 42703509 | mCRPC_PBMC | limma_eBayes | 1 | 7 | 52 | non-coding | (NR_073046, |
| chr22 | 48027541 | mCRPC_PBMC | limma_eBayes | −1 | 24 | 53 | promoter-TSS | (NR_033377) |
| chr1 | 2036183 | NE_Adeno | cellDMC | −1 | 17 | 1 | intron | (NM_001033582, |
| chr1 | 2309984 | NE_Adeno | cellDMC | −1 | 36 | 2 | non-coding | (NR_125361, |
| chr1 | 2379650 | NE_Adeno | cellDMC | −1 | 48 | 3 | Intergenic | |
| chr1 | 9887711 | NE_Adeno | cellDMC | −1 | 6 | 4 | Intergenic | |
| chr1 | 119870187 | NE_Adeno | cellDMC | −1 | 21 | 5 | promoter-TSS | (NR_146623) |
| chr1 | 150122937 | NE_Adeno | cellDMC | −1 | 18 | 6 | 5′ | UTR |
| chr1 | 155034908 | NE_Adeno | cellDMC | −1 | 30 | 7 | promoter-TSS | (NM_182689) |
| chr1 | 156215318 | NE_Adeno | cellDMC | −1 | 43 | 8 | exon | (NM_024897, |
| chr1 | 205631878 | NE_Adeno | cellDMC | 1 | 14 | 9 | exon | (NM_033102, |
| chr1 | 234666987 | NE_Adeno | cellDMC | 1 | 9 | 10 | non-coding | (NR_038426, |
| chr1 | 234908126 | NE_Adeno | cellDMC | −1 | 8 | 11 | Intergenic | |
| chr1 | 235805303 | NE_Adeno | cellDMC | −1 | 47 | 12 | intron | (NM_001098721, |
| chr1 | 246939116 | NE_Adeno | cellDMC | 1 | 1 | 13 | Intergenic | |
| chr2 | 3281230 | NE_Adeno | cellDMC | −1 | 37 | 14 | intron | (NM_001330530, |
| chr2 | 10567426 | NE_Adeno | cellDMC | 1 | 11 | 15 | TTS | (NM_134421) |
| chr2 | 11796686 | NE_Adeno | cellDMC | 1 | 3 | 16 | Intergenic | |
| chr2 | 45237589 | NE_Adeno | cellDMC | −1 | 34 | 17 | Intergenic | |
| chr2 | 121432974 | NE_Adeno | cellDMC | 1 | 25 | 18 | Intergenic | |
| chr2 | 172952783 | NE_Adeno | cellDMC | 1 | 16 | 19 | 3′ | UTR |
| chr2 | 240161519 | NE_Adeno | cellDMC | −1 | 46 | 20 | intron | (NM_006037, |
| chr3 | 134125473 | NE_Adeno | cellDMC | −1 | 27 | 21 | Intergenic | |
| chr3 | 140813669 | NE_Adeno | cellDMC | −1 | 5 | 22 | intron | (NM_080862, |
| chr3 | 160167217 | NE_Adeno | cellDMC | 1 | 30 | 23 | promoter-TSS | (NM_173084) |
| chr3 | 183891828 | NE_Adeno | cellDMC | 1 | 18 | 24 | promoter-TSS | (NM_001311198) |
| chr4 | 981385 | NE_Adeno | cellDMC | 1 | 24 | 25 | 3′ | UTR |
| chr4 | 1205814 | NE_Adeno | cellDMC | 1 | 2 | 26 | non-coding | (NR_104331, |
| chr4 | 7228168 | NE_Adeno | cellDMC | −1 | 7 | 27 | intron | (NM_020777, |
| chr5 | 92938049 | NE_Adeno | cellDMC | −1 | 4 | 28 | Intergenic | |
| chr5 | 134827036 | NE_Adeno | cellDMC | −1 | 10 | 29 | Intergenic | |
| chr5 | 147162061 | NE_Adeno | cellDMC | −1 | 42 | 30 | promoter-TSS | (NM_001282282) |
| chr5 | 179588340 | NE_Adeno | cellDMC | −1 | 29 | 31 | intron | (NM_175062, |
| chr6 | 40567217 | NE_Adeno | cellDMC | 1 | 26 | 32 | Intergenic | |
| chr6 | 170449317 | NE_Adeno | cellDMC | −1 | 41 | 33 | Intergenic | |
| chr6 | 170578492 | NE_Adeno | cellDMC | −1 | 1 | 34 | intron | (NR_126021, |
| chr7 | 214477 | NE_Adeno | cellDMC | 1 | 13 | 35 | intron | (NM_020223, |
| chr7 | 906626 | NE_Adeno | cellDMC | 1 | 6 | 36 | intron | (NR_160282, |
| chr7 | 921951 | NE_Adeno | cellDMC | 1 | 12 | 37 | intron | (NM_015949, |
| chr7 | 2968216 | NE_Adeno | cellDMC | −1 | 3 | 38 | intron | (NM_001324281, |
| chr7 | 50628618 | NE_Adeno | cellDMC | −1 | 25 | 39 | promoter-TSS | (NM_001242889) |
| chr7 | 129912387 | NE_Adeno | cellDMC | −1 | 13 | 40 | intron | (NM_001869, |
| chr7 | 151432920 | NE_Adeno | cellDMC | −1 | 31 | 41 | 5′ | UTR |
| chr8 | 73848837 | NE_Adeno | cellDMC | 1 | 4 | 42 | exon | (NM_004770, |
| chr8 | 93114050 | NE_Adeno | cellDMC | −1 | 24 | 43 | intron | (NM_175634, |
| chr10 | 670687 | NE_Adeno | cellDMC | −1 | 44 | 44 | intron | (NM_014974, |
| chr10 | 123923781 | NE_Adeno | cellDMC | −1 | 33 | 45 | intron | (NM_001291878, |
| chr11 | 20625892 | NE_Adeno | cellDMC | 1 | 28 | 46 | intron | (NM_004211, |
| chr11 | 61449637 | NE_Adeno | cellDMC | −1 | 22 | 47 | intron | (NM_006133, |
| chr11 | 62211890 | NE_Adeno | cellDMC | −1 | 20 | 48 | intron | (NM_024060, |
| chr12 | 104852255 | NE_Adeno | cellDMC | −1 | 12 | 49 | intron | (NM018413, |
| chr12 | 110172108 | NE_Adeno | cellDMC | −1 | 39 | 50 | non-coding | (NR_026661, |
| chr12 | 125578255 | NE_Adeno | cellDMC | −1 | 38 | 51 | intron | (NM_023928, |
| chr14 | 91862713 | NE_Adeno | cellDMC | 1 | 20 | 52 | intron | (NM_001080414, |
| chr14 | 95693684 | NE_Adeno | cellDMC | −1 | 40 | 53 | intron | (NM_024734, |
| chr14 | 101157815 | NE_Adeno | cellDMC | −1 | 49 | 54 | Intergenic | |
| chr15 | 89921572 | NE_Adeno | cellDMC | 1 | 15 | 55 | intron | (NR_133002, |
| chr15 | 89959557 | NE_Adeno | cellDMC | 1 | 29 | 56 | intron | (NR_160724, |
| chr15 | 99194331 | NE_Adeno | cellDMC | 1 | 23 | 57 | promoter-TSS | (NR_126453) |
| chr16 | 1195273 | NE_Adeno | cellDMC | −1 | 45 | 58 | Intergenic | |
| chr16 | 29324459 | NE_Adeno | cellDMC | −1 | 19 | 59 | intron | (NR_002939, |
| chr17 | 59480574 | NE_Adeno | cellDMC | −1 | 15 | 60 | intron | (NM_005994, |
| chr17 | 60827102 | NE_Adeno | cellDMC | −1 | 50 | 61 | intron | (NM_001288779, |
| chr17 | 71744333 | NE_Adeno | cellDMC | −1 | 9 | 62 | TTS | (NR_027146) |
| chr17 | 72350254 | NE_Adeno | cellDMC | 1 | 22 | 63 | exon | (NM_153209, |
| chr17 | 73996145 | NE_Adeno | cellDMC | 1 | 10 | 64 | promoter-TSS | (NR_130467).2 |
| chr17 | 76355046 | NE_Adeno | cellDMC | −1 | 28 | 65 | 5′ | UTR |
| chr17 | 78079381 | NE_Adeno | cellDMC | −1 | 26 | 66 | exon | (NM_000152, |
| chr17 | 79223965 | NE_Adeno | cellDMC | −1 | 11 | 67 | TTS | (NM_138570) |
| chr17 | 80019426 | NE_Adeno | cellDMC | 1 | 5 | 68 | exon | (NM_022156, |
| chr17 | 80052807 | NE_Adeno | cellDMC | 1 | 8 | 69 | intron | (NM_004104, |
| chr17 | 80372675 | NE_Adeno | cellDMC | 1 | 17 | 70 | intron | (NR_033265, |
| chr17 | 80832228 | NE_Adeno | cellDMC | 1 | 19 | 71 | intron | (NM_005993, |
| chr18 | 8659409 | NE_Adeno | cellDMC | −1 | 14 | 72 | Intergenic | |
| chr18 | 13136240 | NE_Adeno | cellDMC | −1 | 35 | 73 | Intergenic | |
| chr19 | 2540807 | NE_Adeno | cellDMC | 1 | 7 | 74 | intron | (NM_052847, |
| chr19 | 4566300 | NE_Adeno | cellDMC | −1 | 32 | 75 | Intergenic | |
| chr19 | 17790974 | NE_Adeno | cellDMC | 1 | 27 | 76 | intron | (NM_001080421, |
| chr20 | 20432962 | NE_Adeno | cellDMC | −1 | 2 | 77 | intron | (NM_020343, |
| chr21 | 51589516 | NE_Adeno | cellDMC | −1 | 23 | 78 | 5′ | UTR |
| chr21 | 38077158 | NE_Adeno | cellDMC | 1 | 21 | 79 | intron | (NM_009586, |
| chr21 | 43441927 | NE_Adeno | cellDMC | −1 | 16 | 80 | promoter-TSS | (NR_119385) |
| chr1 | 2036220 | rockermeth | rockermethSegs | −1 | 398 | 1 | promoter-TSS | (NM_001033582) |
| chr1 | 2036226 | rockermeth | rockermethSegs | −1 | 400 | 2 | promoter-TSS | (NM_001033582) |
| chr1 | 2036232 | rockermeth | rockermethSegs | −1 | 397 | 3 | promoter-TSS | (NM_001033582) |
| chr1 | 2036252 | rockermeth | rockermethSegs | −1 | 399 | 4 | promoter-TSS | (NM_001033582) |
| chr1 | 2036395 | rockermeth | rockermethSegs | −1 | 401 | 5 | intron | (NM_001033582, |
| chr1 | 3697617 | rockermeth | rockermethSegs | 1 | 68 | 6 | 3′ | UTR |
| chr1 | 3697629 | rockermeth | rockermethSegs | 1 | 63 | 7 | 3′ | UTR |
| chr1 | 3697639 | rockermeth | rockermethSegs | 1 | 64 | 8 | 3′ | UTR |
| chr1 | 3697648 | rockermeth | rockermethSegs | 1 | 65 | 9 | 3′ | UTR |
| chr1 | 3697654 | rockermeth | rockermethSegs | 1 | 66 | 10 | exon | (NM_020710, |
| chr1 | 3697667 | rockermeth | rockermethSegs | 1 | 67 | 11 | exon | (NM_020710, |
| chr1 | 3697692 | rockermeth | rockermethSegs | 1 | 70 | 12 | exon | (NM_020710, |
| chr1 | 3697702 | rockermeth | rockermethSegs | 1 | 69 | 13 | exon | (NM_020710, |
| chr1 | 6171653 | rockermeth | rockermethSegs | −1 | 515 | 14 | intron | (NM_015557, |
| chr1 | 6171833 | rockermeth | rockermethSegs | −1 | 514 | 15 | exon | (NM_015557, |
| chr1 | 6171838 | rockermeth | rockermethSegs | −1 | 511 | 16 | exon | (NM_015557, |
| chr1 | 6171845 | rockermeth | rockermethSegs | −1 | 513 | 17 | exon | (NM_015557, |
| chr1 | 6171867 | rockermeth | rockermethSegs | −1 | 512 | 18 | exon | (NM_015557, |
| chr1 | 9501648 | rockermeth | rockermethSegs | −1 | 362 | 19 | Intergenic | |
| chr1 | 10696021 | rockermeth | rockermethSegs | 1 | 86 | 20 | TTS | (NM_001079843) |
| chr1 | 10696065 | rockermeth | rockermethSegs | 1 | 87 | 21 | TTS | (NM_001079843) |
| chr1 | 18808323 | rockermeth | rockermethSegs | −1 | 107 | 22 | exon | (NM_152375, |
| chr1 | 18808343 | rockermeth | rockermethSegs | −1 | 118 | 23 | exon | (NM_152375, |
| chr1 | 18808439 | rockermeth | rockermethSegs | −1 | 120 | 24 | exon | (NM_152375, |
| chr1 | 18808469 | rockermeth | rockermethSegs | −1 | 111 | 25 | exon | (NM_152375, |
| chr1 | 18808546 | rockermeth | rockermethSegs | −1 | 124 | 26 | exon | (NM_152375, |
| chr1 | 18808583 | rockermeth | rockermethSegs | −1 | 113 | 27 | exon | (NM_152375, |
| chr1 | 18808621 | rockermeth | rockermethSegs | −1 | 123 | 28 | exon | (NM_152375, |
| chr1 | 18808832 | rockermeth | rockermethSegs | −1 | 125 | 29 | exon | (NM_152375, |
| chr1 | 18808880 | rockermeth | rockermethSegs | −1 | 119 | 30 | exon | (NM_152375, |
| chr1 | 18808929 | rockermeth | rockermethSegs | −1 | 126 | 31 | exon | (NM_152375, |
| chr1 | 18808938 | rockermeth | rockermethSegs | −1 | 109 | 32 | exon | (NM_152375, |
| chr1 | 18808967 | rockermeth | rockermethSegs | −1 | 110 | 33 | exon | (NM_152375, |
| chr1 | 18808991 | rockermeth | rockermethSegs | −1 | 127 | 34 | exon | (NM_152375, |
| chr1 | 18809009 | rockermeth | rockermethSegs | −1 | 116 | 35 | exon | (NM_152375, |
| chr1 | 18809028 | rockermeth | rockermethSegs | −1 | 115 | 36 | exon | (NM_152375, |
| chr1 | 18809041 | rockermeth | rockermethSegs | −1 | 114 | 37 | exon | (NM_152375, |
| chr1 | 18809052 | rockermeth | rockermethSegs | −1 | 129 | 38 | exon | (NM_152375, |
| chr1 | 18809058 | rockermeth | rockermethSegs | −1 | 121 | 39 | exon | (NM_152375, |
| chr1 | 18809064 | rockermeth | rockermethSegs | −1 | 112 | 40 | exon | (NM_152375, |
| chr1 | 18809102 | rockermeth | rockermethSegs | −1 | 108 | 41 | exon | (NM_152375, |
| chr1 | 18809112 | rockermeth | rockermethSegs | −1 | 128 | 42 | exon | (NM_152375, |
| chr1 | 18809126 | rockermeth | rockermethSegs | −1 | 117 | 43 | exon | (NM_152375, |
| chr1 | 18809494 | rockermeth | rockermethSegs | −1 | 122 | 44 | exon | (NM_152375, |
| chr1 | 20141125 | rockermeth | rockermethSegs | −1 | 266 | 45 | exon | (NM_019062, |
| chr1 | 20141136 | rockermeth | rockermethSegs | −1 | 267 | 46 | exon | (NM_019062, |
| chr1 | 20141322 | rockermeth | rockermethSegs | −1 | 268 | 47 | exon | (NM_019062, |
| chr1 | 20141344 | rockermeth | rockermethSegs | −1 | 269 | 48 | exon | (NM_019062, |
| chr1 | 20141404 | rockermeth | rockermethSegs | −1 | 271 | 49 | exon | (NM_019062, |
| chr1 | 20141442 | rockermeth | rockermethSegs | −1 | 265 | 50 | exon | (NM_019062, |
| chr1 | 20141462 | rockermeth | rockermethSegs | −1 | 270 | 51 | exon | (NM_019062, |
| chr1 | 22195529 | rockermeth | rockermethSegs | −1 | 612 | 52 | intron | (NM_005529, |
| chr1 | 32252163 | rockermeth | rockermethSegs | −1 | 105 | 53 | Intergenic | |
| chr1 | 32252229 | rockermeth | rockermethSegs | −1 | 104 | 54 | Intergenic | |
| chr1 | 32252239 | rockermeth | rockermethSegs | −1 | 103 | 55 | Intergenic | |
| chr1 | 32252264 | rockermeth | rockermethSegs | −1 | 106 | 56 | Intergenic | |
| chr1 | 32252425 | rockermeth | rockermethSegs | −1 | 102 | 57 | Intergenic | |
| chr1 | 45103348 | rockermeth | rockermethSegs | −1 | 450 | 58 | intron | (NM_001319957, |
| chr1 | 45103390 | rockermeth | rockermethSegs | −1 | 449 | 59 | intron | (NM_001319957, |
| chr1 | 45106491 | rockermeth | rockermethSegs | −1 | 451 | 60 | intron | (NM_001319957, |
| chr1 | 156215343 | rockermeth | rockermethSegs | −1 | 717 | 61 | exon | (NM_001272106, |
| chr1 | 156215371 | rockermeth | rockermethSegs | −1 | 721 | 62 | exon | (NM_001272106, |
| chr1 | 156215401 | rockermeth | rockermethSegs | −1 | 713 | 63 | exon | (NM_001272106, |
| chr1 | 156215416 | rockermeth | rockermethSegs | −1 | 703 | 64 | exon | (NM_001272106, |
| chr1 | 156215430 | rockermeth | rockermethSegs | −1 | 715 | 65 | exon | (NM_001272106, |
| chr1 | 156215445 | rockermeth | rockermethSegs | −1 | 709 | 66 | exon | (NM_024897, |
| chr1 | 156215454 | rockermeth | rockermethSegs | −1 | 711 | 67 | intron | (NM_198406, |
| chr1 | 156215491 | rockermeth | rockermethSegs | −1 | 708 | 68 | intron | (NM_198406, |
| chr1 | 156215506 | rockermeth | rockermethSegs | −1 | 716 | 69 | intron | (NM_198406, |
| chr1 | 156215520 | rockermeth | rockermethSegs | −1 | 710 | 70 | intron | (NM_198406, |
| chr1 | 156215525 | rockermeth | rockermethSegs | −1 | 714 | 71 | intron | (NM_198406, |
| chr1 | 156215530 | rockermeth | rockermethSegs | −1 | 720 | 72 | intron | (NM_198406, |
| chr1 | 156215563 | rockermeth | rockermethSegs | −1 | 706 | 73 | intron | (NM_198406, |
| chr1 | 156215590 | rockermeth | rockermethSegs | −1 | 704 | 74 | exon | (NM_024897, |
| chr1 | 156215601 | rockermeth | rockermethSegs | −1 | 705 | 75 | exon | (NM_024897, |
| chr1 | 156215708 | rockermeth | rockermethSegs | −1 | 719 | 76 | non-coding | (NR_073610, |
| chr1 | 156215720 | rockermeth | rockermethSegs | −1 | 718 | 77 | non-coding | (NR_073610, |
| chr1 | 156215726 | rockermeth | rockermethSegs | −1 | 707 | 78 | non-coding | (NR_073610, |
| chr1 | 156215737 | rockermeth | rockermethSegs | −1 | 712 | 79 | non-coding | (NR_073610, |
| chr1 | 203259747 | rockermeth | rockermethSegs | −1 | 402 | 80 | Intergenic | |
| chr1 | 203259757 | rockermeth | rockermethSegs | −1 | 406 | 81 | Intergenic | |
| chr1 | 203259770 | rockermeth | rockermethSegs | −1 | 403 | 82 | Intergenic | |
| chr1 | 203259836 | rockermeth | rockermethSegs | −1 | 405 | 83 | Intergenic | |
| chr1 | 203259850 | rockermeth | rockermethSegs | −1 | 407 | 84 | Intergenic | |
| chr1 | 203259921 | rockermeth | rockermethSegs | −1 | 404 | 85 | Intergenic | |
| chr1 | 236311219 | rockermeth | rockermethSegs | 1 | 108 | 86 | intron | (NM_003272, |
| chr1 | 236311342 | rockermeth | rockermethSegs | 1 | 109 | 87 | intron | (NM_003272, |
| chr1 | 237129554 | rockermeth | rockermethSegs | −1 | 495 | 88 | Intergenic | |
| chr1 | 237134163 | rockermeth | rockermethSegs | −1 | 501 | 89 | Intergenic | |
| chr1 | 237167480 | rockermeth | rockermethSegs | −1 | 500 | 90 | TTS | (NM_001276687) |
| chr1 | 237194084 | rockermeth | rockermethSegs | −1 | 502 | 91 | Intergenic | |
| chr1 | 237194093 | rockermeth | rockermethSegs | −1 | 499 | 92 | Intergenic | |
| chr1 | 237194098 | rockermeth | rockermethSegs | −1 | 497 | 93 | Intergenic | |
| chr1 | 237195314 | rockermeth | rockermethSegs | −1 | 498 | 94 | Intergenic | |
| chr1 | 237195411 | rockermeth | rockermethSegs | −1 | 496 | 95 | Intergenic | |
| chr1 | 237212336 | rockermeth | rockermethSegs | −1 | 808 | 96 | intron | (NM_001035, |
| chr1 | 237220609 | rockermeth | rockermethSegs | −1 | 795 | 97 | intron | (NM_001035, |
| chr1 | 237220641 | rockermeth | rockermethSegs | −1 | 774 | 98 | intron | (NM_001035, |
| chr1 | 237222098 | rockermeth | rockermethSegs | −1 | 778 | 99 | intron | (NM_001035, |
| chr1 | 237222106 | rockermeth | rockermethSegs | −1 | 802 | 100 | intron | (NM_001035, |
| chr1 | 237235121 | rockermeth | rockermethSegs | −1 | 775 | 101 | intron | (NM_001035, |
| chr1 | 237235204 | rockermeth | rockermethSegs | −1 | 806 | 102 | intron | (NM_001035, |
| chr1 | 237235522 | rockermeth | rockermethSegs | −1 | 791 | 103 | intron | (NM_001035, |
| chr1 | 237235567 | rockermeth | rockermethSegs | −1 | 777 | 104 | intron | (NM_001035, |
| chr1 | 237235607 | rockermeth | rockermethSegs | −1 | 804 | 105 | intron | (NM_001035, |
| chr1 | 237256273 | rockermeth | rockermethSegs | −1 | 805 | 106 | intron | (NM_001035, |
| chr1 | 237256316 | rockermeth | rockermethSegs | −1 | 781 | 107 | intron | (NM_001035, |
| chr1 | 237265786 | rockermeth | rockermethSegs | −1 | 809 | 108 | intron | (NM_001035, |
| chr1 | 237265877 | rockermeth | rockermethSegs | −1 | 783 | 109 | intron | (NM_001035, |
| chr1 | 237267786 | rockermeth | rockermethSegs | −1 | 784 | 110 | intron | (NM_001035, |
| chr1 | 237267812 | rockermeth | rockermethSegs | −1 | 785 | 111 | intron | (NM_001035, |
| chr1 | 237267886 | rockermeth | rockermethSegs | −1 | 790 | 112 | intron | (NM_001035, |
| chr1 | 237306208 | rockermeth | rockermethSegs | −1 | 792 | 113 | intron | (NM_001035, |
| chr1 | 237306260 | rockermeth | rockermethSegs | −1 | 782 | 114 | intron | (NM_001035, |
| chr1 | 237343254 | rockermeth | rockermethSegs | −1 | 779 | 115 | intron | (NM_001035, |
| chr1 | 237359169 | rockermeth | rockermethSegs | −1 | 810 | 116 | intron | (NM_001035, |
| chr1 | 237383690 | rockermeth | rockermethSegs | −1 | 773 | 117 | intron | (NM_001035, |
| chr1 | 237392760 | rockermeth | rockermethSegs | −1 | 799 | 118 | intron | (NM_001035, |
| chr1 | 237408076 | rockermeth | rockermethSegs | −1 | 788 | 119 | intron | (NM_001035, |
| chr1 | 237408091 | rockermeth | rockermethSegs | −1 | 803 | 120 | intron | (NM_001035, |
| chr1 | 237473057 | rockermeth | rockermethSegs | −1 | 776 | 121 | intron | (NM_001035, |
| chr1 | 237473071 | rockermeth | rockermethSegs | −1 | 789 | 122 | intron | (NM_001035, |
| chr1 | 237473091 | rockermeth | rockermethSegs | −1 | 786 | 123 | intron | (NM_001035, |
| chr1 | 237473164 | rockermeth | rockermethSegs | −1 | 798 | 124 | intron | (NM_001035, |
| chr1 | 237538162 | rockermeth | rockermethSegs | −1 | 800 | 125 | intron | (NM_001035, |
| chr1 | 237584276 | rockermeth | rockermethSegs | −1 | 771 | 126 | intron | (NM_001035, |
| chr1 | 237661252 | rockermeth | rockermethSegs | −1 | 793 | 127 | intron | (NM_001035, |
| chr1 | 237661262 | rockermeth | rockermethSegs | −1 | 801 | 128 | intron | (NM_001035, |
| chr1 | 237661322 | rockermeth | rockermethSegs | −1 | 770 | 129 | intron | (NM_001035, |
| chr1 | 237693526 | rockermeth | rockermethSegs | −1 | 787 | 130 | intron | (NM_001035, |
| chr1 | 237693534 | rockermeth | rockermethSegs | −1 | 772 | 131 | intron | (NM_001035, |
| chr1 | 237699930 | rockermeth | rockermethSegs | −1 | 794 | 132 | intron | (NM_001035, |
| chr1 | 237699938 | rockermeth | rockermethSegs | −1 | 780 | 133 | intron | (NM_001035, |
| chr1 | 237699988 | rockermeth | rockermethSegs | −1 | 807 | 134 | intron | (NM_001035, |
| chr1 | 237809365 | rockermeth | rockermethSegs | −1 | 796 | 135 | intron | (NM_001035, |
| chr1 | 237815260 | rockermeth | rockermethSegs | −1 | 797 | 136 | intron | (NM_001035, |
| chr1 | 241402493 | rockermeth | rockermethSegs | 1 | 91 | 137 | intron | (NM_001364886, |
| chr1 | 241402516 | rockermeth | rockermethSegs | 1 | 92 | 138 | intron | (NM_001364886, |
| chr2 | 9248023 | rockermeth | rockermethSegs | −1 | 259 | 139 | Intergenic | |
| chr2 | 9248368 | rockermeth | rockermethSegs | −1 | 258 | 140 | Intergenic | |
| chr2 | 9248435 | rockermeth | rockermethSegs | −1 | 260 | 141 | Intergenic | |
| chr2 | 9248469 | rockermeth | rockermethSegs | −1 | 257 | 142 | Intergenic | |
| chr2 | 25299679 | rockermeth | rockermethSegs | −1 | 591 | 143 | intron | (NM_001319099, |
| chr2 | 25299815 | rockermeth | rockermethSegs | −1 | 590 | 144 | intron | (NM_001319099, |
| chr2 | 25299928 | rockermeth | rockermethSegs | −1 | 589 | 145 | intron | (NM_001319099, |
| chr2 | 43102420 | rockermeth | rockermethSegs | 1 | 97 | 146 | Intergenic | |
| chr2 | 43107259 | rockermeth | rockermethSegs | 1 | 93 | 147 | Intergenic | |
| chr2 | 43107400 | rockermeth | rockermethSegs | 1 | 98 | 148 | Intergenic | |
| chr2 | 43107414 | rockermeth | rockermethSegs | 1 | 96 | 149 | Intergenic | |
| chr2 | 43107438 | rockermeth | rockermethSegs | 1 | 99 | 150 | Intergenic | |
| chr2 | 43107456 | rockermeth | rockermethSegs | 1 | 95 | 151 | Intergenic | |
| chr2 | 43107465 | rockermeth | rockermethSegs | 1 | 94 | 152 | Intergenic | |
| chr2 | 43107514 | rockermeth | rockermethSegs | 1 | 101 | 153 | Intergenic | |
| chr2 | 43107532 | rockermeth | rockermethSegs | 1 | 102 | 154 | Intergenic | |
| chr2 | 43107650 | rockermeth | rockermethSegs | 1 | 100 | 155 | Intergenic | |
| chr2 | 45235684 | rockermeth | rockermethSegs | −1 | 262 | 156 | intron | (NM_016932, |
| chr2 | 45235702 | rockermeth | rockermethSegs | −1 | 263 | 157 | exon | (NM_016932, |
| chr2 | 45235710 | rockermeth | rockermethSegs | −1 | 264 | 158 | exon | (NM_016932, |
| chr2 | 207972376 | rockermeth | rockermethSegs | −1 | 208 | 159 | intron | (NR_073108, |
| chr2 | 207972408 | rockermeth | rockermethSegs | −1 | 207 | 160 | intron | (NR_073108, |
| chr2 | 220509654 | rockermeth | rockermethSegs | −1 | 336 | 161 | Intergenic | |
| chr2 | 220509693 | rockermeth | rockermethSegs | −1 | 339 | 162 | Intergenic | |
| chr2 | 220509705 | rockermeth | rockermethSegs | −1 | 338 | 163 | Intergenic | |
| chr2 | 220509776 | rockermeth | rockermethSegs | −1 | 337 | 164 | Intergenic | |
| chr2 | 220509830 | rockermeth | rockermethSegs | −1 | 335 | 165 | Intergenic | |
| chr2 | 241233035 | rockermeth | rockermethSegs | −1 | 177 | 166 | Intergenic | |
| chr2 | 241233048 | rockermeth | rockermethSegs | −1 | 176 | 167 | Intergenic | |
| chr2 | 241233055 | rockermeth | rockermethSegs | −1 | 174 | 168 | Intergenic | |
| chr2 | 241233084 | rockermeth | rockermethSegs | −1 | 171 | 169 | Intergenic | |
| chr2 | 241233090 | rockermeth | rockermethSegs | −1 | 175 | 170 | Intersenic | |
| chr2 | 241233169 | rockermeth | rockermethSegs | −1 | 172 | 171 | Intergenic | |
| chr2 | 241233214 | rockermeth | rockermethSegs | −1 | 173 | 172 | Intergenic | |
| chr3 | 111431516 | rockermeth | rockermethSegs | −1 | 226 | 173 | intron | (NM_001185106, |
| chr3 | 111431559 | rockermeth | rockermethSegs | −1 | 225 | 174 | intron | (NM_001185106, |
| chr4 | 3309964 | rockermeth | rockermethSegs | −1 | 91 | 175 | Intergenic | |
| chr4 | 3310004 | rockermeth | rockermethSegs | −1 | 90 | 176 | Intergenic | |
| chr4 | 3311787 | rockermeth | rockermethSegs | −1 | 93 | 177 | Intergenic | |
| chr4 | 3311917 | rockermeth | rockermethSegs | −1 | 92 | 178 | Intergenic | |
| chr4 | 7228254 | rockermeth | rockermethSegs | −1 | 88 | 179 | intron | (NM_020777, |
| chr4 | 7228263 | rockermeth | rockermethSegs | −1 | 86 | 180 | intron | (NM_020777, |
| chr4 | 7228304 | rockermeth | rockermethSegs | −1 | 87 | 181 | intron | (NM_020777, |
| chr4 | 7228329 | rockermeth | rockermethSegs | −1 | 89 | 182 | intron | (NM_020777, |
| chr4 | 7326173 | rockermeth | rockermethSegs | −1 | 660 | 183 | intron | (NM_020777, |
| chr4 | 7326188 | rockermeth | rockermethSegs | −1 | 661 | 184 | intron | (NM_020777, |
| chr4 | 7326270 | rockermeth | rockermethSegs | −1 | 659 | 185 | intron | (NM_020777, |
| chr4 | 7326284 | rockermeth | rockermethSegs | −1 | 662 | 186 | intron | (NM_020777, |
| chr4 | 155758143 | rockermeth | rockermethSegs | −1 | 646 | 187 | Intergenic | |
| chr4 | 155763679 | rockermeth | rockermethSegs | −1 | 649 | 188 | Intergenic | |
| chr4 | 155763688 | rockermeth | rockermethSegs | −1 | 647 | 189 | Intergenic | |
| chr4 | 155763695 | rockermeth | rockermethSegs | −1 | 650 | 190 | Intergenic | |
| chr4 | 155812513 | rockermeth | rockermethSegs | −1 | 651 | 191 | Intergenic | |
| chr4 | 155912924 | rockermeth | rockermethSegs | −1 | 639 | 192 | Intergenic | |
| chr4 | 155965520 | rockermeth | rockermethSegs | −1 | 641 | 193 | Intergenic | |
| chr4 | 155965532 | rockermeth | rockermethSegs | −1 | 643 | 194 | Intergenic | |
| chr4 | 155965538 | rockermeth | rockermethSegs | −1 | 644 | 195 | Intergenic | |
| chr4 | 155965544 | rockermeth | rockermethSegs | −1 | 640 | 196 | Intergenic | |
| chr4 | 155965553 | rockermeth | rockermethSegs | −1 | 645 | 197 | Intergenic | |
| chr4 | 155992200 | rockermeth | rockermethSegs | −1 | 648 | 198 | Intergenic | |
| chr4 | 156002533 | rockermeth | rockermethSegs | −1 | 642 | 199 | Intergenic | |
| chr5 | 1003895 | rockermeth | rockermethSegs | −1 | 747 | 200 | Intergenic | |
| chr5 | 1004045 | rockermeth | rockermethSegs | −1 | 742 | 201 | Intergenic | |
| chr5 | 1004051 | rockermeth | rockermethSegs | −1 | 735 | 202 | Intergenic | |
| chr5 | 1004064 | rockermeth | rockermethSegs | −1 | 736 | 203 | Intergenic | |
| chr5 | 1004077 | rockermeth | rockermethSegs | −1 | 738 | 204 | Intergenic | |
| chr5 | 1004089 | rockermeth | rockermethSegs | −1 | 740 | 205 | Intergenic | |
| chr5 | 1004118 | rockermeth | rockermethSegs | −1 | 730 | 206 | Intergenic | |
| chr5 | 1004128 | rockermeth | rockermethSegs | −1 | 731 | 207 | Intergenic | |
| chr5 | 1004135 | rockermeth | rockermethSegs | −1 | 732 | 208 | Intergenic | |
| chr5 | 1004148 | rockermeth | rockermethSegs | −1 | 728 | 209 | Intergenic | |
| chr5 | 1004157 | rockermeth | rockermethSegs | −1 | 729 | 210 | Intergenic | |
| chr5 | 1004184 | rockermeth | rockermethSegs | −1 | 733 | 211 | Intergenic | |
| chr5 | 1004198 | rockermeth | rockermethSegs | −1 | 745 | 212 | Intergenic | |
| chr5 | 1004234 | rockermeth | rockermethSegs | −1 | 739 | 213 | Intergenic | |
| chr5 | 1004243 | rockermeth | rockermethSegs | −1 | 734 | 214 | Intergenic | |
| chr5 | 1004259 | rockermeth | rockermethSegs | −1 | 743 | 215 | Intergenic | |
| chr5 | 1004269 | rockermeth | rockermethSegs | −1 | 748 | 216 | Intergenic | |
| chr5 | 1004288 | rockermeth | rockermethSegs | −1 | 749 | 217 | Intergenic | |
| chr5 | 1004304 | rockermeth | rockermethSegs | −1 | 737 | 218 | Intergenic | |
| chr5 | 1004351 | rockermeth | rockermethSegs | −1 | 746 | 219 | Intergenic | |
| chr5 | 1004357 | rockermeth | rockermethSegs | −1 | 741 | 220 | Intergenic | |
| chr5 | 1004365 | rockermeth | rockermethSegs | −1 | 744 | 221 | Intergenic | |
| chr5 | 134827336 | rockermeth | rockermethSegs | −1 | 216 | 222 | Intergenic | |
| chr5 | 134827353 | rockermeth | rockermethSegs | −1 | 217 | 223 | Intergenic | |
| chr5 | 134827364 | rockermeth | rockermethSegs | −1 | 218 | 224 | Intergenic | |
| chr5 | 134827378 | rockermeth | rockermethSegs | −1 | 219 | 225 | Intergenic | |
| chr5 | 138441347 | rockermeth | rockermethSegs | −1 | 1 | 226 | intron | (NM_001037633, |
| chr5 | 138441377 | rockermeth | rockermethSegs | −1 | 2 | 227 | intron | (NM_001037633, |
| chr5 | 138441561 | rockermeth | rockermethSegs | −1 | 3 | 228 | intron | (NM_001037633, |
| chr5 | 179401802 | rockermeth | rockermethSegs | −1 | 50 | 229 | intron | (NM_018434, |
| chr5 | 179401833 | rockermeth | rockermethSegs | −1 | 54 | 230 | intron | (NM_018434, |
| chr5 | 179401843 | rockermeth | rockermethSegs | −1 | 53 | 231 | intron | (NM_018434, |
| chr5 | 179401853 | rockermeth | rockermethSegs | −1 | 52 | 232 | intron | (NM_018434, |
| chr5 | 179401872 | rockermeth | rockermethSegs | −1 | 51 | 233 | intron | (NM_018434, |
| chr5 | 179588495 | rockermeth | rockermethSegs | −1 | 463 | 234 | intron | (NM_175062, |
| chr5 | 179588504 | rockermeth | rockermethSegs | −1 | 465 | 235 | intron | (NM_175062, |
| chr5 | 179588512 | rockermeth | rockermethSegs | −1 | 464 | 236 | intron | (NM_175062, |
| chr5 | 179588528 | rockermeth | rockermethSegs | −1 | 455 | 237 | intron | (NM_175062, |
| chr5 | 179588550 | rockermeth | rockermethSegs | −1 | 454 | 238 | intron | (NM_175062, |
| chr5 | 179588590 | rockermeth | rockermethSegs | −1 | 457 | 239 | intron | (NM_175062, |
| chr5 | 179588604 | rockermeth | rockermethSegs | −1 | 456 | 240 | intron | (NM_175062, |
| chr5 | 179588617 | rockermeth | rockermethSegs | −1 | 458 | 241 | intron | (NM_175062, |
| chr5 | 179588625 | rockermeth | rockermethSegs | −1 | 459 | 242 | intron | (NM_175062, |
| chr5 | 179588631 | rockermeth | rockermethSegs | −1 | 461 | 243 | intron | (NM_175062, |
| chr5 | 179588672 | rockermeth | rockermethSegs | −1 | 469 | 244 | intron | (NM_175062, |
| chr5 | 179588687 | rockermeth | rockermethSegs | −1 | 466 | 245 | intron | (NM_175062, |
| chr5 | 179588704 | rockermeth | rockermethSegs | −1 | 467 | 246 | intron | (NM_175062, |
| chr5 | 179588719 | rockermeth | rockermethSegs | −1 | 460 | 247 | intron | (NM_175062, |
| chr5 | 179597660 | rockermeth | rockermethSegs | −1 | 468 | 248 | intron | (NM_175062, |
| chr5 | 179597771 | rockermeth | rockermethSegs | −1 | 470 | 249 | intron | (NM_175062, |
| chr5 | 179597812 | rockermeth | rockermethSegs | −1 | 462 | 250 | intron | (NM_175062, |
| chr6 | 64151793 | rockermeth | rockermethSegs | −1 | 769 | 251 | Intergenic | |
| chr6 | 157549787 | rockermeth | rockermethSegs | −1 | 385 | 252 | Intergenic | |
| chr6 | 157549797 | rockermeth | rockermethSegs | −1 | 383 | 253 | Intergenic | |
| chr6 | 157549848 | rockermeth | rockermethSegs | −1 | 384 | 254 | Intergenic | |
| chr6 | 157550494 | rockermeth | rockermethSegs | −1 | 387 | 255 | Intergenic | |
| chr6 | 157550509 | rockermeth | rockermethSegs | −1 | 386 | 256 | Intergenic | |
| chr7 | 1447696 | rockermeth | rockermethSegs | −1 | 64 | 257 | Intergenic | |
| chr7 | 1447734 | rockermeth | rockermethSegs | −1 | 65 | 258 | Intergenic | |
| chr7 | 1447746 | rockermeth | rockermethSegs | −1 | 67 | 259 | Intergenic | |
| chr7 | 1447760 | rockermeth | rockermethSegs | −1 | 69 | 260 | Intergenic | |
| chr7 | 1447774 | rockermeth | rockermethSegs | −1 | 68 | 261 | Intergenic | |
| chr7 | 1447782 | rockermeth | rockermethSegs | −1 | 66 | 262 | Intergenic | |
| chr7 | 2006576 | rockermeth | rockermethSegs | −1 | 203 | 263 | intron | (NM_001304523, |
| chr7 | 2006674 | rockermeth | rockermethSegs | −1 | 202 | 264 | intron | (NM_001304523, |
| chr7 | 2007262 | rockermeth | rockermethSegs | −1 | 201 | 265 | intron | (NM_001304523, |
| chr7 | 2007279 | rockermeth | rockermethSegs | −1 | 204 | 266 | intron | (NM_001304523, |
| chr7 | 2007309 | rockermeth | rockermethSegs | −1 | 206 | 267 | intron | (NM_001304523, |
| chr7 | 2007394 | rockermeth | rockermethSegs | −1 | 205 | 268 | intron | (NM_001304523, |
| chr7 | 2968280 | rockermeth | rockermethSegs | −1 | 35 | 269 | exon | (NM_032415, |
| chr7 | 2968288 | rockermeth | rockermethSegs | −1 | 28 | 270 | exon | (NM_032415, |
| chr7 | 2968314 | rockermeth | rockermethSegs | −1 | 15 | 271 | exon | (NM_032415, |
| chr7 | 2968320 | rockermeth | rockermethSegs | −1 | 32 | 272 | exon | (NM_032415, |
| chr7 | 2968352 | rockermeth | rockermethSegs | −1 | 36 | 273 | intron | (NM_001324281, |
| chr7 | 2968357 | rockermeth | rockermethSegs | −1 | 33 | 274 | intron | (NM_001324281, |
| chr7 | 2968405 | rockermeth | rockermethSegs | −1 | 29 | 275 | intron | (NM_001324281, |
| chr7 | 2968411 | rockermeth | rockermethSegs | −1 | 16 | 276 | intron | (NM_001324281, |
| chr7 | 2968426 | rockermeth | rockermethSegs | −1 | 17 | 277 | intron | (NM_001324281, |
| chr7 | 2968440 | rockermeth | rockermethSegs | −1 | 18 | 278 | intron | (NM_001324281, |
| chr7 | 2968454 | rockermeth | rockermethSegs | −1 | 19 | 279 | intron | (NM_001324281, |
| chr7 | 2968489 | rockermeth | rockermethSegs | −1 | 20 | 280 | intron | (NM_001324281, |
| chr7 | 2968504 | rockermeth | rockermethSegs | −1 | 34 | 281 | intron | (NM_001324281, |
| chr7 | 2968528 | rockermeth | rockermethSegs | −1 | 21 | 282 | intron | (NM_001324281, |
| chr7 | 2968552 | rockermeth | rockermethSegs | −1 | 22 | 283 | intron | (NM_001324281, |
| chr7 | 2968558 | rockermeth | rockermethSegs | −1 | 23 | 284 | intron | (NM_001324281, |
| chr7 | 2968565 | rockermeth | rockermethSegs | −1 | 24 | 285 | intron | (NM_001324281, |
| chr7 | 2968585 | rockermeth | rockermethSegs | −1 | 25 | 286 | intron | (NM_001324281, |
| chr7 | 2968593 | rockermeth | rockermethSegs | −1 | 26 | 287 | intron | (NM_001324281, |
| chr7 | 2968625 | rockermeth | rockermethSegs | −1 | 27 | 288 | intron | (NM_001324281, |
| chr7 | 2968684 | rockermeth | rockermethSegs | −1 | 30 | 289 | intron | (NM_001324281, |
| chr7 | 2968706 | rockermeth | rockermethSegs | −1 | 31 | 290 | intron | (NM_001324281, |
| chr7 | 26667584 | rockermeth | rockermethSegs | −1 | 300 | 291 | Intergenic | |
| chr7 | 26667613 | rockermeth | rockermethSegs | −1 | 303 | 292 | Intergenic | |
| chr7 | 26667674 | rockermeth | rockermethSegs | −1 | 301 | 293 | Intergenic | |
| chr7 | 26667709 | rockermeth | rockermethSegs | −1 | 302 | 294 | Intergenic | |
| chr7 | 35505816 | rockermeth | rockermethSegs | −1 | 701 | 295 | Intergenic | |
| chr7 | 35505825 | rockermeth | rockermethSegs | −1 | 702 | 296 | Intergenic | |
| chr7 | 41086116 | rockermeth | rockermethSegs | −1 | 504 | 297 | Intergenic | |
| chr7 | 41086123 | rockermeth | rockermethSegs | −1 | 503 | 298 | Intergenic | |
| chr7 | 41086140 | rockermeth | rockermethSegs | −1 | 505 | 299 | Intergenic | |
| chr7 | 101622017 | rockermeth | rockermethSegs | −1 | 84 | 300 | intron | (NM_001202544, |
| chr7 | 101622057 | rockermeth | rockermethSegs | −1 | 85 | 301 | intron | (NM_001202544, |
| chr7 | 101622176 | rockermeth | rockermethSegs | −1 | 83 | 302 | intron | (NM_001202544, |
| chr7 | 115992730 | rockermeth | rockermethSegs | −1 | 343 | 303 | Intergenic | |
| chr7 | 115992814 | rockermeth | rockermethSegs | −1 | 341 | 304 | Intergenic | |
| chr7 | 115998982 | rockermeth | rockermethSegs | −1 | 340 | 305 | Intergenic | |
| chr7 | 115999026 | rockermeth | rockermethSegs | −1 | 342 | 306 | Intergenic | |
| chr7 | 115999448 | rockermeth | rockermethSegs | −1 | 345 | 307 | Intergenic | |
| chr7 | 115999464 | rockermeth | rockermethSegs | −1 | 346 | 308 | Intergenic | |
| chr7 | 116099337 | rockermeth | rockermethSegs | −1 | 344 | 309 | Intergenic | |
| chr7 | 150668588 | rockermeth | rockermethSegs | −1 | 452 | 310 | intron | (NM_000238, |
| chr7 | 150669120 | rockermeth | rockermethSegs | −1 | 453 | 311 | intron | (NM_000238, |
| chr7 | 150811991 | rockermeth | rockermethSegs | 1 | 72 | 312 | exon | (NM_001308305, |
| chr7 | 150812075 | rockermeth | rockermethSegs | 1 | 76 | 313 | exon | (NM_001308305, |
| chr7 | 150812111 | rockermeth | rockermethSegs | 1 | 77 | 314 | exon | (NM_001308305, |
| chr7 | 150812201 | rockermeth | rockermethSegs | 1 | 74 | 315 | exon | (NM_001308305, |
| chr7 | 150812215 | rockermeth | rockermethSegs | 1 | 73 | 316 | exon | (NM_001308305, |
| chr7 | 150812398 | rockermeth | rockermethSegs | 1 | 75 | 317 | exon | (NM_001308305, |
| chr7 | 150812660 | rockermeth | rockermethSegs | 1 | 71 | 318 | exon | (NM_001308305, |
| chr7 | 157423275 | rockermeth | rockermethSegs | 1 | 1 | 319 | intron | (NM_001308268, |
| chr7 | 157623246 | rockermeth | rockermethSegs | −1 | 170 | 320 | intron | (NM_001308268, |
| chr8 | 46929775 | rockermeth | rockermethSegs | −1 | 359 | 321 | Intergenic | |
| chr8 | 46935836 | rockermeth | rockermethSegs | −1 | 353 | 322 | Intergenic | |
| chr8 | 46938035 | rockermeth | rockermethSegs | −1 | 354 | 323 | Intergenic | |
| chr8 | 46938044 | rockermeth | rockermethSegs | −1 | 361 | 324 | Intergenic | |
| chr8 | 46938657 | rockermeth | rockermethSegs | −1 | 355 | 325 | Intergenic | |
| chr8 | 46938693 | rockermeth | rockermethSegs | −1 | 356 | 326 | Intergenic | |
| chr8 | 46938702 | rockermeth | rockermethSegs | −1 | 357 | 327 | Intergenic | |
| chr8 | 46940361 | rockermeth | rockermethSegs | −1 | 358 | 328 | Intergenic | |
| chr8 | 46940406 | rockermeth | rockermethSegs | −1 | 360 | 329 | Intergenic | |
| chr8 | 58062363 | rockermeth | rockermethSegs | −1 | 672 | 330 | Intergenic | |
| chr8 | 58062426 | rockermeth | rockermethSegs | −1 | 666 | 331 | Intergenic | |
| chr8 | 58062463 | rockermeth | rockermethSegs | −1 | 670 | 332 | Intergenic | |
| chr8 | 58096948 | rockermeth | rockermethSegs | −1 | 665 | 333 | Intergenic | |
| chr8 | 58115010 | rockermeth | rockermethSegs | −1 | 695 | 334 | Intergenic | |
| chr8 | 58115060 | rockermeth | rockermethSegs | −1 | 686 | 335 | Intergenic | |
| chr8 | 58115154 | rockermeth | rockermethSegs | −1 | 697 | 336 | Intergenic | |
| chr8 | 58115225 | rockermeth | rockermethSegs | −1 | 680 | 337 | Intergenic | |
| chr8 | 58115343 | rockermeth | rockermethSegs | −1 | 663 | 338 | Intergenic | |
| chr8 | 58115362 | rockermeth | rockermethSegs | −1 | 698 | 339 | Intergenic | |
| chr8 | 58122819 | rockermeth | rockermethSegs | −1 | 693 | 340 | Intergenic | |
| chr8 | 58122836 | rockermeth | rockermethSegs | −1 | 676 | 341 | Intergenic | |
| chr8 | 58122848 | rockermeth | rockermethSegs | −1 | 690 | 342 | Intergenic | |
| chr8 | 58122861 | rockermeth | rockermethSegs | −1 | 682 | 343 | Intergenic | |
| chr8 | 58123008 | rockermeth | rockermethSegs | −1 | 684 | 344 | Intergenic | |
| chr8 | 58126620 | rockermeth | rockermethSegs | −1 | 691 | 345 | Intergenic | |
| chr8 | 58126666 | rockermeth | rockermethSegs | −1 | 700 | 346 | Intergenic | |
| chr8 | 58126874 | rockermeth | rockermethSegs | −1 | 675 | 347 | Intergenic | |
| chr8 | 58127096 | rockermeth | rockermethSegs | −1 | 668 | 348 | Intergenic | |
| chr8 | 58127515 | rockermeth | rockermethSegs | −1 | 685 | 349 | Intergenic | |
| chr8 | 58127556 | rockermeth | rockermethSegs | −1 | 664 | 350 | Intergenic | |
| chr8 | 58127613 | rockermeth | rockermethSegs | −1 | 674 | 351 | Intergenic | |
| chr8 | 58130004 | rockermeth | rockermethSegs | −1 | 678 | 352 | promoter-TSS | (NR_038235) |
| chr8 | 58130021 | rockermeth | rockermethSegs | −1 | 699 | 353 | promoter-TSS | (NR_038235) |
| chr8 | 58130039 | rockermeth | rockermethSegs | −1 | 677 | 354 | promoter-TSS | (NR_038235) |
| chr8 | 58131654 | rockermeth | rockermethSegs | −1 | 667 | 355 | non-coding | (NR_038235, |
| chr8 | 58131665 | rockermeth | rockermethSegs | −1 | 673 | 356 | non-coding | (NR_038235, |
| chr8 | 58131710 | rockermeth | rockermethSegs | −1 | 689 | 357 | intron | (NR_038235, |
| chr8 | 58131715 | rockermeth | rockermethSegs | −1 | 669 | 358 | intron | (NR_038235, |
| chr8 | 58131843 | rockermeth | rockermethSegs | −1 | 687 | 359 | intron | (NR_038235, |
| chr8 | 58131870 | rockermeth | rockermethSegs | −1 | 696 | 360 | intron | (NR_038235, |
| chr8 | 58133184 | rockermeth | rockermethSegs | −1 | 688 | 361 | intron | (NR_038235, |
| chr8 | 58133215 | rockermeth | rockermethSegs | −1 | 671 | 362 | intron | (NR_038235, |
| chr8 | 58133315 | rockermeth | rockermethSegs | −1 | 694 | 363 | intron | (NR_038235, |
| chr8 | 58133743 | rockermeth | rockermethSegs | −1 | 681 | 364 | intron | (NR_038235, |
| chr8 | 58171196 | rockermeth | rockermethSegs | −1 | 692 | 365 | Intergenic | |
| chr8 | 58171236 | rockermeth | rockermethSegs | −1 | 683 | 366 | Intergenic | |
| chr8 | 58192020 | rockermeth | rockermethSegs | −1 | 679 | 367 | promoter-TSS | (NR_026772) |
| chr8 | 61777862 | rockermeth | rockermethSegs | −1 | 165 | 368 | exon | (NM_001316690, |
| chr8 | 61821531 | rockermeth | rockermethSegs | −1 | 162 | 369 | Intergenic | |
| chr8 | 61821546 | rockermeth | rockermethSegs | −1 | 163 | 370 | Intergenic | |
| chr8 | 61821578 | rockermeth | rockermethSegs | −1 | 164 | 371 | Intergenic | |
| chr8 | 73471538 | rockermeth | rockermethSegs | 1 | 81 | 372 | intron | (NM_004770, |
| chr8 | 73480053 | rockermeth | rockermethSegs | 1 | 82 | 373 | exon | (NM_004770, |
| chr8 | 73551842 | rockermeth | rockermethSegs | 1 | 80 | 374 | intron | (NM_004770, |
| chr8 | 73849013 | rockermeth | rockermethSegs | 1 | 85 | 375 | exon | (NM_004770, |
| chr8 | 73849032 | rockermeth | rockermethSegs | 1 | 84 | 376 | exon | (NM_004770, |
| chr8 | 73849038 | rockermeth | rockermethSegs | 1 | 83 | 377 | exon | (NM_004770, |
| chr8 | 73849051 | rockermeth | rockermethSegs | 1 | 78 | 378 | exon | (NM_004770, |
| chr8 | 73849060 | rockermeth | rockermethSegs | 1 | 79 | 379 | exon | (NM_004770, |
| chr8 | 104117894 | rockermeth | rockermethSegs | −1 | 352 | 380 | Intergenic | |
| chr8 | 140929837 | rockermeth | rockermethSegs | −1 | 604 | 381 | intron | (NM_001160372, |
| chr8 | 140930939 | rockermeth | rockermethSegs | −1 | 606 | 382 | intron | (NM_001160372, |
| chr8 | 140931316 | rockermeth | rockermethSegs | −1 | 605 | 383 | intron | (NM_001160372, |
| chr8 | 142367903 | rockermeth | rockermethSegs | 1 | 10 | 384 | exon | (NM_005293, |
| chr8 | 142367951 | rockermeth | rockermethSegs | 1 | 12 | 385 | exon | (NM_005293, |
| chr8 | 142367989 | rockermeth | rockermethSegs | 1 | 11 | 386 | exon | (NM_005293, |
| chr9 | 117049844 | rockermeth | rockermethSegs | −1 | 636 | 387 | intron | (NM_032888, |
| chr9 | 117049850 | rockermeth | rockermethSegs | −1 | 638 | 388 | intron | (NM_032888, |
| chr9 | 117049862 | rockermeth | rockermethSegs | −1 | 635 | 389 | intron | (NM_032888, |
| chr9 | 117049885 | rockermeth | rockermethSegs | −1 | 637 | 390 | intron | (NM_032888, |
| chr9 | 127105722 | rockermeth | rockermethSegs | −1 | 516 | 391 | intron | (NM_014397, |
| chr9 | 127105763 | rockermeth | rockermethSegs | −1 | 517 | 392 | intron | (NM_014397, |
| chr9 | 129895923 | rockermeth | rockermethSegs | −1 | 139 | 393 | intron | (NM_014636, |
| chr9 | 136007375 | rockermeth | rockermethSegs | −1 | 322 | 394 | promoter-TSS | (NM_001271774) |
| chr9 | 136007484 | rockermeth | rockermethSegs | −1 | 319 | 395 | promoter-TSS | (NM_001271774) |
| chr9 | 136007594 | rockermeth | rockermethSegs | −1 | 320 | 396 | intron | (NM_001042368, |
| chr9 | 136007599 | rockermeth | rockermethSegs | −1 | 321 | 397 | intron | (NM_001042368, |
| chr9 | 136007615 | rockermeth | rockermethSegs | −1 | 324 | 398 | intron | (NM_001042368, |
| chr9 | 136007712 | rockermeth | rockermethSegs | −1 | 323 | 399 | intron | (NM_001042368, |
| chr9 | 136324027 | rockermeth | rockermethSegs | −1 | 519 | 400 | intron | (NM_139026, |
| chr9 | 136324037 | rockermeth | rockermethSegs | −1 | 520 | 401 | intron | (NM_139026, |
| chr9 | 136324064 | rockermeth | rockermethSegs | −1 | 518 | 402 | intron | (NM_139026, |
| chr9 | 138688868 | rockermeth | rockermethSegs | −1 | 654 | 403 | Intergenic | |
| chr9 | 138688876 | rockermeth | rockermethSegs | −1 | 652 | 404 | Intergenic | |
| chr9 | 138688902 | rockermeth | rockermethSegs | −1 | 653 | 405 | Intergenic | |
| chr9 | 138688936 | rockermeth | rockermethSegs | −1 | 656 | 406 | Intergenic | |
| chr9 | 138689094 | rockermeth | rockermethSegs | −1 | 657 | 407 | Intergenic | |
| chr9 | 138689107 | rockermeth | rockermethSegs | −1 | 655 | 408 | Intergenic | |
| chr9 | 138689483 | rockermeth | rockermethSegs | −1 | 658 | 409 | Intergenic | |
| chr9 | 138719312 | rockermeth | rockermethSegs | 1 | 13 | 410 | exon | (NM_015447, |
| chr9 | 138719386 | rockermeth | rockermethSegs | 1 | 14 | 411 | exon | (NM_015447, |
| chr9 | 139419669 | rockermeth | rockermethSegs | −1 | 288 | 412 | intron | (NM_017617, |
| chr9 | 139419769 | rockermeth | rockermethSegs | −1 | 286 | 413 | intron | (NM_017617, |
| chr9 | 139419814 | rockermeth | rockermethSegs | −1 | 298 | 414 | intron | (NM_017617, |
| chr9 | 139419865 | rockermeth | rockermethSegs | −1 | 278 | 415 | intron | (NM_017617, |
| chr9 | 139419924 | rockermeth | rockermethSegs | −1 | 299 | 416 | intron | (NM_017617, |
| chr9 | 139419933 | rockermeth | rockermethSegs | −1 | 275 | 417 | intron | (NM_017617, |
| chr9 | 139419956 | rockermeth | rockermethSegs | −1 | 287 | 418 | intron | (NM_017617, |
| chr9 | 139420550 | rockermeth | rockermethSegs | −1 | 294 | 419 | intron | (NM_017617, |
| chr9 | 139420561 | rockermeth | rockermethSegs | −1 | 297 | 420 | intron | (NM_017617, |
| chr9 | 139420578 | rockermeth | rockermethSegs | −1 | 289 | 421 | intron | (NM_017617, |
| chr9 | 139420602 | rockermeth | rockermethSegs | −1 | 274 | 422 | intron | (NM_017617, |
| chr9 | 139420641 | rockermeth | rockermethSegs | −1 | 282 | 423 | intron | (NM_017617, |
| chr9 | 139420862 | rockermeth | rockermethSegs | −1 | 284 | 424 | intron | (NM_017617, |
| chr9 | 139420914 | rockermeth | rockermethSegs | −1 | 276 | 425 | intron | (NM_017617, |
| chr9 | 139421144 | rockermeth | rockermethSegs | −1 | 295 | 426 | intron | (NM_017617, |
| chr9 | 139421220 | rockermeth | rockermethSegs | −1 | 291 | 427 | intron | (NM_017617, |
| chr9 | 139421241 | rockermeth | rockermethSegs | −1 | 296 | 428 | intron | (NM_017617, |
| chr9 | 139421257 | rockermeth | rockermethSegs | −1 | 280 | 429 | intron | (NM_017617, |
| chr9 | 139421267 | rockermeth | rockermethSegs | −1 | 293 | 430 | intron | (NM_017617, |
| chr9 | 139421321 | rockermeth | rockermethSegs | −1 | 285 | 431 | intron | (NM_017617, |
| chr9 | 139421342 | rockermeth | rockermethSegs | −1 | 292 | 432 | intron | (NM_017617, |
| chr9 | 139421362 | rockermeth | rockermethSegs | −1 | 290 | 433 | intron | (NM_017617, |
| chr9 | 139422178 | rockermeth | rockermethSegs | −1 | 272 | 434 | intron | (NM_017617, |
| chr9 | 139422203 | rockermeth | rockermethSegs | −1 | 279 | 435 | intron | (NM_017617, |
| chr9 | 139422218 | rockermeth | rockermethSegs | −1 | 277 | 436 | intron | (NM_017617, |
| chr9 | 139422266 | rockermeth | rockermethSegs | −1 | 281 | 437 | intron | (NM_017617, |
| chr9 | 139422290 | rockermeth | rockermethSegs | −1 | 273 | 438 | intron | (NM_017617, |
| chr9 | 139422314 | rockermeth | rockermethSegs | −1 | 283 | 439 | intron | (NM_017617, |
| chr10 | 23976532 | rockermeth | rockermethSegs | −1 | 623 | 440 | Intergenic | |
| chr10 | 23976581 | rockermeth | rockermethSegs | −1 | 624 | 441 | Intergenic | |
| chr10 | 72489844 | rockermeth | rockermethSegs | −1 | 13 | 442 | intron | (NM_139155, |
| chr10 | 72489870 | rockermeth | rockermethSegs | −1 | 9 | 443 | exon | (NM_080722, |
| chr10 | 72489889 | rockermeth | rockermethSegs | −1 | 10 | 444 | exon | (NM_080722, |
| chr10 | 72489949 | rockermeth | rockermethSegs | −1 | 12 | 445 | exon | (NM_080722, |
| chr10 | 72489967 | rockermeth | rockermethSegs | −1 | 11 | 446 | exon | (NM_080722, |
| chr10 | 72489983 | rockermeth | rockermethSegs | −1 | 14 | 447 | exon | (NM_080722, |
| chr10 | 119590052 | rockermeth | rockermethSegs | −1 | 325 | 448 | Intergenic | |
| chr10 | 134562610 | rockermeth | rockermethSegs | −1 | 390 | 449 | intron | (NM_005539, |
| chr10 | 134563052 | rockermeth | rockermethSegs | −1 | 388 | 450 | exon | (NM_001321042, |
| chr10 | 134563060 | rockermeth | rockermethSegs | −1 | 393 | 451 | exon | (NM_001321042, |
| chr10 | 134563098 | rockermeth | rockermethSegs | −1 | 391 | 452 | exon | (NM_001321042, |
| chr10 | 134563109 | rockermeth | rockermethSegs | −1 | 394 | 453 | exon | (NM_001321042, |
| chr10 | 134563144 | rockermeth | rockermethSegs | −1 | 395 | 454 | intron | (NM_005539, |
| chr10 | 134563149 | rockermeth | rockermethSegs | −1 | 392 | 455 | intron | (NM_005539, |
| chr10 | 134563186 | rockermeth | rockermethSegs | −1 | 396 | 456 | intron | (NM_005539, |
| chr10 | 134563199 | rockermeth | rockermethSegs | −1 | 389 | 457 | intron | (NM_005539, |
| chr11 | 2495501 | rockermeth | rockermethSegs | −1 | 601 | 458 | intron | (NM_181798, |
| chr11 | 2495543 | rockermeth | rockermethSegs | −1 | 602 | 459 | intron | (NM_181798, |
| chr11 | 2495585 | rockermeth | rockermethSegs | −1 | 603 | 460 | intron | (NM_181798, |
| chr11 | 2495609 | rockermeth | rockermethSegs | −1 | 600 | 461 | intron | (NM_181798, |
| chr11 | 68967954 | rockermeth | rockermethSegs | −1 | 565 | 462 | Intergenic | |
| chr11 | 68967994 | rockermeth | rockermethSegs | −1 | 563 | 463 | Intergenic | |
| chr11 | 68968016 | rockermeth | rockermethSegs | −1 | 562 | 464 | Intergenic | |
| chr11 | 68968032 | rockermeth | rockermethSegs | −1 | 564 | 465 | Intergenic | |
| chr11 | 68968042 | rockermeth | rockermethSegs | −1 | 569 | 466 | Intergenic | |
| chr11 | 68968141 | rockermeth | rockermethSegs | −1 | 566 | 467 | Intergenic | |
| chr11 | 68968151 | rockermeth | rockermethSegs | −1 | 567 | 468 | Intergenic | |
| chr11 | 68968156 | rockermeth | rockermethSegs | −1 | 568 | 469 | Intergenic | |
| chr11 | 68968182 | rockermeth | rockermethSegs | −1 | 561 | 470 | Intergenic | |
| chr11 | 68968214 | rockermeth | rockermethSegs | −1 | 570 | 471 | Intergenic | |
| chr11 | 68968240 | rockermeth | rockermethSegs | −1 | 571 | 472 | Intergenic | |
| chr11 | 68968267 | rockermeth | rockermethSegs | −1 | 560 | 473 | Intergenic | |
| chr11 | 70851529 | rockermeth | rockermethSegs | −1 | 316 | 474 | intron | (NM_012309, |
| chr11 | 70851574 | rockermeth | rockermethSegs | −1 | 314 | 475 | intron | (NM_012309, |
| chr11 | 70851603 | rockermeth | rockermethSegs | −1 | 309 | 476 | intron | (NM_012309, |
| chr11 | 70851619 | rockermeth | rockermethSegs | −1 | 307 | 477 | intron | (NM_012309, |
| chr11 | 70851638 | rockermeth | rockermethSegs | −1 | 312 | 478 | intron | (NM_012309, |
| chr11 | 70851713 | rockermeth | rockermethSegs | −1 | 306 | 479 | intron | (NM_012309, |
| chr11 | 70851722 | rockermeth | rockermethSegs | −1 | 317 | 480 | intron | (NM_012309, |
| chr11 | 70851730 | rockermeth | rockermethSegs | −1 | 310 | 481 | intron | (NM_012309, |
| chr11 | 70851827 | rockermeth | rockermethSegs | −1 | 311 | 482 | intron | (NM_012309, |
| chr11 | 70851836 | rockermeth | rockermethSegs | −1 | 313 | 483 | intron | (NM_012309, |
| chr11 | 70851859 | rockermeth | rockermethSegs | −1 | 308 | 484 | intron | (NM_012309, |
| chr11 | 70851868 | rockermeth | rockermethSegs | −1 | 304 | 485 | intron | (NM_012309, |
| chr11 | 70851883 | rockermeth | rockermethSegs | −1 | 318 | 486 | intron | (NM_012309, |
| chr11 | 70851889 | rockermeth | rockermethSegs | −1 | 305 | 487 | intron | (NM_012309, |
| chr11 | 70851914 | rockermeth | rockermethSegs | −1 | 315 | 488 | intron | (NM_012309, |
| chr11 | 70862301 | rockermeth | rockermethSegs | −1 | 166 | 489 | intron | (NM_012309, |
| chr11 | 70862379 | rockermeth | rockermethSegs | −1 | 168 | 490 | intron | (NM_012309, |
| chr11 | 70862397 | rockermeth | rockermethSegs | −1 | 167 | 491 | intron | (NM_012309, |
| chr11 | 70862405 | rockermeth | rockermethSegs | −1 | 169 | 492 | intron | (NM_012309, |
| chr11 | 111265961 | rockermeth | rockermethSegs | −1 | 382 | 493 | Intergenic | |
| chr11 | 111266000 | rockermeth | rockermethSegs | −1 | 378 | 494 | Intergenic | |
| chr11 | 111266014 | rockermeth | rockermethSegs | −1 | 381 | 495 | Intergenic | |
| chr11 | 111266031 | rockermeth | rockermethSegs | −1 | 380 | 496 | Intergenic | |
| chr11 | 111266119 | rockermeth | rockermethSegs | −1 | 379 | 497 | Intergenic | |
| chr11 | 115829517 | rockermeth | rockermethSegs | −1 | 328 | 498 | Intergenic | |
| chr11 | 115829550 | rockermeth | rockermethSegs | −1 | 329 | 499 | Intergenic | |
| chr11 | 115830020 | rockermeth | rockermethSegs | −1 | 327 | 500 | Intergenic | |
| chr11 | 115830200 | rockermeth | rockermethSegs | −1 | 326 | 501 | Intergenic | |
| chr11 | 133902554 | rockermeth | rockermethSegs | −1 | 580 | 502 | non-coding | (NR_027276, |
| chr11 | 133902596 | rockermeth | rockermethSegs | −1 | 587 | 503 | non-coding | (NR_027276, |
| chr11 | 133902615 | rockermeth | rockermethSegs | −1 | 588 | 504 | non-coding | (NR_027276, |
| chr11 | 133902626 | rockermeth | rockermethSegs | −1 | 585 | 505 | non-coding | (NR_027276, |
| chr11 | 133902656 | rockermeth | rockermethSegs | −1 | 582 | 506 | non-coding | (NR_027276, |
| chr11 | 133902669 | rockermeth | rockermethSegs | −1 | 583 | 507 | non-coding | (NR_027276, |
| chr11 | 133902689 | rockermeth | rockermethSegs | −1 | 581 | 508 | non-coding | (NR_027276, |
| chr11 | 133902724 | rockermeth | rockermethSegs | −1 | 579 | 509 | non-coding | (NR_027276. |
| chr11 | 133902737 | rockermeth | rockermethSegs | −1 | 584 | 510 | non-coding | (NR_027276, |
| chr11 | 133902744 | rockermeth | rockermethSegs | −1 | 586 | 511 | non-coding | (NR_027276, |
| chr11 | 133902766 | rockermeth | rockermethSegs | −1 | 578 | 512 | intron | (NR_027276, |
| chr12 | 1957456 | rockermeth | rockermethSegs | −1 | 508 | 513 | intron | (NM_172364, |
| chr12 | 1957466 | rockermeth | rockermethSegs | −1 | 507 | 514 | intron | (NM_172364, |
| chr12 | 1957486 | rockermeth | rockermethSegs | −1 | 506 | 515 | intron | (NM_172364, |
| chr12 | 1957501 | rockermeth | rockermethSegs | −1 | 510 | 516 | intron | (NM_172364, |
| chr12 | 1957566 | rockermeth | rockermethSegs | −1 | 509 | 517 | intron | (NM_172364, |
| chr12 | 34488345 | rockermeth | rockermethSegs | 1 | 107 | 518 | Intergenic | |
| chr12 | 34488352 | rockermeth | rockermethSegs | 1 | 106 | 519 | Intergenic | |
| chr12 | 66111480 | rockermeth | rockermethSegs | −1 | 350 | 520 | Intergenic | |
| chr12 | 66111548 | rockermeth | rockermethSegs | −1 | 348 | 521 | Intergenic | |
| chr12 | 66114657 | rockermeth | rockermethSegs | −1 | 347 | 522 | Intergenic | |
| chr12 | 66114687 | rockermeth | rockermethSegs | −1 | 351 | 523 | Intergenic | |
| chr12 | 66114695 | rockermeth | rockermethSegs | −1 | 349 | 524 | Intergenic | |
| chr12 | 66262212 | rockermeth | rockermethSegs | −1 | 131 | 525 | intron | (NR_158984, |
| chr12 | 66262228 | rockermeth | rockermethSegs | −1 | 130 | 526 | intron | (NR_158984, |
| chr12 | 115109512 | rockermeth | rockermethSegs | 1 | 5 | 527 | 3′ | UTR |
| chr12 | 115109529 | rockermeth | rockermethSegs | 1 | 7 | 528 | 3′ | UTR |
| chr12 | 115109718 | rockermeth | rockermethSegs | 1 | 6 | 529 | exon | (NM_005996, |
| chr12 | 115109960 | rockermeth | rockermethSegs | 1 | 2 | 530 | exon | (NM_005996, |
| chr12 | 115112024 | rockermeth | rockermethSegs | 1 | 4 | 531 | exon | (NM_005996, |
| chr12 | 115112037 | rockermeth | rockermethSegs | 1 | 3 | 532 | exon | (NM_005996, |
| chr12 | 124947867 | rockermeth | rockermethSegs | −1 | 143 | 533 | intron | (NM_001206654, |
| chr12 | 124948139 | rockermeth | rockermethSegs | −1 | 144 | 534 | intron | (NM_001206654, |
| chr13 | 114523664 | rockermeth | rockermethSegs | −1 | 559 | 535 | 3′ | UTR |
| chr13 | 114523669 | rockermeth | rockermethSegs | −1 | 549 | 536 | 3′ | UTR |
| chr13 | 114523717 | rockermeth | rockermethSegs | −1 | 543 | 537 | 3′ | UTR |
| chr13 | 114523760 | rockermeth | rockermethSegs | −1 | 536 | 538 | 3′ | UTR |
| chr13 | 114523787 | rockermeth | rockermethSegs | −1 | 537 | 539 | 3′ | UTR |
| chr13 | 114523796 | rockermeth | rockermethSegs | −1 | 533 | 540 | 3′ | UTR |
| chr13 | 114523819 | rockermeth | rockermethSegs | −1 | 538 | 541 | 3′ | UTR |
| chr13 | 114523826 | rockermeth | rockermethSegs | −1 | 544 | 542 | 3′ | UTR |
| chr13 | 114523875 | rockermeth | rockermethSegs | −1 | 550 | 543 | exon | (NM_000820, |
| chr13 | 114523883 | rockermeth | rockermethSegs | −1 | 535 | 544 | exon | (NM_000820, |
| chr13 | 114523896 | rockermeth | rockermethSegs | −1 | 531 | 545 | exon | (NM_000820, |
| chr13 | 114523904 | rockermeth | rockermethSegs | −1 | 546 | 546 | exon | (NM_000820, |
| chr13 | 114523923 | rockermeth | rockermethSegs | −1 | 532 | 547 | exon | (NM_000820, |
| chr13 | 114523948 | rockermeth | rockermethSegs | −1 | 530 | 548 | exon | (NM_000820, |
| chr13 | 114523958 | rockermeth | rockermethSegs | −1 | 542 | 549 | exon | (NM_000820, |
| chr13 | 114523970 | rockermeth | rockermethSegs | −1 | 534 | 550 | exon | (NM_000820, |
| chr13 | 114523982 | rockermeth | rockermethSegs | −1 | 556 | 551 | exon | (NM_000820, |
| chr13 | 114523996 | rockermeth | rockermethSegs | −1 | 539 | 552 | intron | (NM_000820, |
| chr13 | 114524029 | rockermeth | rockermethSegs | −1 | 548 | 553 | intron | (NM_000820, |
| chr13 | 114524093 | rockermeth | rockermethSegs | −1 | 529 | 554 | intron | (NM_000820, |
| chr13 | 114524130 | rockermeth | rockermethSegs | −1 | 558 | 555 | intron | (NM_000820, |
| chr13 | 114524137 | rockermeth | rockermethSegs | −1 | 553 | 556 | intron | (NM_000820, |
| chr13 | 114524149 | rockermeth | rockermethSegs | −1 | 557 | 557 | intron | (NM_000820, |
| chr13 | 114524157 | rockermeth | rockermethSegs | −1 | 554 | 558 | intron | (NM_000820, |
| chr13 | 114524420 | rockermeth | rockermethSegs | −1 | 540 | 559 | intron | (NM_000820, |
| chr13 | 114524429 | rockermeth | rockermethSegs | −1 | 541 | 560 | intron | (NM_000820, |
| chr13 | 114524439 | rockermeth | rockermethSegs | −1 | 552 | 561 | intron | (NM_000820, |
| chr13 | 114524473 | rockermeth | rockermethSegs | −1 | 547 | 562 | intron | (NM_000820, |
| chr3 | 114524509 | rockermeth | rockermethSegs | −1 | 551 | 563 | intron | (NM_000820, |
| chr13 | 114524517 | rockermeth | rockermethSegs | −1 | 545 | 564 | intron | (NM_000820, |
| chr13 | 114524583 | rockermeth | rockermethSegs | −1 | 555 | 565 | intron | (NM_000820, |
| chr14 | 20684518 | rockermeth | rockermethSegs | −1 | 527 | 566 | Intergenic | |
| chr14 | 20684528 | rockermeth | rockermethSegs | −1 | 528 | 567 | Intergenic | |
| chr14 | 76957115 | rockermeth | rockermethSegs | −1 | 153 | 568 | intron | (NM_004452, |
| chr14 | 76957138 | rockermeth | rockermethSegs | −1 | 154 | 569 | intron | (NM_004452, |
| chr14 | 76957152 | rockermeth | rockermethSegs | −1 | 151 | 570 | intron | (NM_004452, |
| chr14 | 76957199 | rockermeth | rockermethSegs | −1 | 152 | 571 | intron | (NM_004452, |
| chr14 | 93423461 | rockermeth | rockermethSegs | −1 | 186 | 572 | intron | (NM_001142593, |
| chr14 | 93423503 | rockermeth | rockermethSegs | −1 | 189 | 573 | intron | (NM_001142593, |
| chr14 | 93423558 | rockermeth | rockermethSegs | −1 | 192 | 574 | intron | (NM_001142593, |
| chr14 | 93425210 | rockermeth | rockermethSegs | −1 | 199 | 575 | intron | (NM_001142593, |
| chr14 | 93425284 | rockermeth | rockermethSegs | −1 | 187 | 576 | intron | (NM_001142593, |
| chr14 | 93425331 | rockermeth | rockermethSegs | −1 | 200 | 577 | intron | (NM_001142593, |
| chr14 | 93425520 | rockermeth | rockermethSegs | −1 | 195 | 578 | intron | (NM_001142593, |
| chr14 | 93425529 | rockermeth | rockermethSegs | −1 | 180 | 579 | intron | (NM_001142593, |
| chr14 | 93425655 | rockermeth | rockermethSegs | −1 | 178 | 580 | intron | (NM_001142593, |
| chr14 | 93425687 | rockermeth | rockermethSegs | −1 | 194 | 581 | intron | (NM_001142593, |
| chr14 | 93425735 | rockermeth | rockermethSegs | −1 | 181 | 582 | intron | (NM_001142593, |
| chr14 | 93425741 | rockermeth | rockermethSegs | −1 | 188 | 583 | intron | (NM_001142593, |
| chr14 | 93426221 | rockermeth | rockermethSegs | −1 | 191 | 584 | intron | (NM_001142593, |
| chr14 | 93426229 | rockermeth | rockermethSegs | −1 | 184 | 585 | intron | (NM_001142593, |
| chr14 | 93426247 | rockermeth | rockermethSegs | −1 | 193 | 586 | intron | (NM_001142593, |
| chr14 | 93426257 | rockermeth | rockermethSegs | −1 | 190 | 587 | intron | (NM_001142593, |
| chr14 | 93426336 | rockermeth | rockermethSegs | −1 | 182 | 588 | intron | (NM_001142593, |
| chr14 | 93426381 | rockermeth | rockermethSegs | −1 | 198 | 589 | intron | (NM_001142593, |
| chr14 | 93426456 | rockermeth | rockermethSegs | −1 | 179 | 590 | intron | (NM_001142593, |
| chr14 | 93426481 | rockermeth | rockermethSegs | −1 | 185 | 591 | intron | (NM_001142593, |
| chr14 | 93426499 | rockermeth | rockermethSegs | −1 | 183 | 592 | intron | (NM_001142593, |
| chr14 | 93426875 | rockermeth | rockermethSegs | −1 | 196 | 593 | intron | (NM_001142593, |
| chr14 | 93426916 | rockermeth | rockermethSegs | −1 | 197 | 594 | intron | (NM_001142593, |
| chr14 | 101158277 | rockermeth | rockermethSegs | −1 | 525 | 595 | Intergenic | |
| chr14 | 101158294 | rockermeth | rockermethSegs | −1 | 524 | 596 | Intergenic | |
| chr14 | 101159404 | rockermeth | rockermethSegs | −1 | 523 | 597 | Intergenic | |
| chr14 | 101159430 | rockermeth | rockermethSegs | −1 | 522 | 598 | Intergenic | |
| chr14 | 101159551 | rockermeth | rockermethSegs | −1 | 521 | 599 | Intergenic | |
| chr14 | 101250153 | rockermeth | rockermethSegs | −1 | 59 | 600 | Intergenic | |
| chr14 | 101250165 | rockermeth | rockermethSegs | −1 | 58 | 601 | Intergenic | |
| chr14 | 101250240 | rockermeth | rockermethSegs | −1 | 56 | 602 | Intergenic | |
| chr14 | 101250254 | rockermeth | rockermethSegs | −1 | 57 | 603 | Intergenic | |
| chr14 | 101250279 | rockermeth | rockermethSegs | −1 | 55 | 604 | Intergenic | |
| chr14 | 101640895 | rockermeth | rockermethSegs | −1 | 40 | 605 | Intergenic | |
| chr14 | 101640910 | rockermeth | rockermethSegs | −1 | 37 | 606 | Intergenic | |
| chr14 | 101640942 | rockermeth | rockermethSegs | −1 | 38 | 607 | Intergenic | |
| chr14 | 101640959 | rockermeth | rockermethSegs | −1 | 39 | 608 | Intergenic | |
| chr14 | 105347685 | rockermeth | rockermethSegs | −1 | 95 | 609 | intron | (NM_001112726, |
| chr14 | 105347700 | rockermeth | rockermethSegs | −1 | 99 | 610 | intron | (NM_001112726, |
| chr14 | 105347712 | rockermeth | rockermethSegs | −1 | 96 | 611 | intron | (NM_001112726, |
| chr14 | 105347802 | rockermeth | rockermethSegs | −1 | 101 | 612 | intron | (NM_001112726, |
| chr14 | 105347810 | rockermeth | rockermethSegs | −1 | 94 | 613 | intron | (NM_001112726, |
| chr14 | 105347834 | rockermeth | rockermethSegs | −1 | 100 | 614 | intron | (NM_001112726, |
| chr14 | 105347851 | rockermeth | rockermethSegs | −1 | 97 | 615 | intron | (NM_001112726, |
| chr14 | 105347961 | rockermeth | rockermethSegs | −1 | 98 | 616 | intron | (NM_001112726, |
| chr15 | 22559959 | rockermeth | rockermethSegs | −1 | 753 | 617 | intron | (NR_033735, |
| chr15 | 22559966 | rockermeth | rockermethSegs | −1 | 752 | 618 | intron | (NR_033735, |
| chr15 | 22567152 | rockermeth | rockermethSegs | −1 | 755 | 619 | non-coding | (NR_033735, |
| chr15 | 22567172 | rockermeth | rockermethSegs | −1 | 750 | 620 | non-coding | (NR_033735, |
| chr15 | 22571751 | rockermeth | rockermethSegs | −1 | 757 | 621 | TTS | (NR_033735) |
| chr15 | 22571783 | rockermeth | rockermethSegs | −1 | 756 | 622 | TTS | (NR_033735) |
| chr15 | 22581845 | rockermeth | rockermethSegs | −1 | 754 | 623 | Intergenic | |
| chr15 | 22581981 | rockermeth | rockermethSegs | −1 | 751 | 624 | Intergenic | |
| chr15 | 74521143 | rockermeth | rockermethSegs | −1 | 232 | 625 | Intergenic | |
| chr15 | 74521156 | rockermeth | rockermethSegs | −1 | 230 | 626 | Intergenic | |
| chr15 | 74521297 | rockermeth | rockermethSegs | −1 | 231 | 627 | Intergenic | |
| chr15 | 74521344 | rockermeth | rockermethSegs | −1 | 227 | 628 | Intergenic | |
| chr15 | 74521357 | rockermeth | rockermethSegs | −1 | 228 | 629 | Intergenic | |
| chr15 | 74521364 | rockermeth | rockermethSegs | −1 | 229 | 630 | Intergenic | |
| chr15 | 94613752 | rockermeth | rockermethSegs | −1 | 330 | 631 | intron | (NR_120320, |
| chr15 | 94613766 | rockermeth | rockermethSegs | −1 | 334 | 632 | intron | (NR_120320, |
| chr15 | 94613787 | rockermeth | rockermethSegs | −1 | 333 | 633 | intron | (NR_120320, |
| chr15 | 94613823 | rockermeth | rockermethSegs | −1 | 331 | 634 | intron | (NR_120320, |
| chr15 | 94613901 | rockermeth | rockermethSegs | −1 | 332 | 635 | intron | (NR_120320, |
| chr17 | 915606 | rockermeth | rockermethSegs | −1 | 432 | 636 | intron | (NM_001256847, |
| chr17 | 915643 | rockermeth | rockermethSegs | −1 | 429 | 637 | intron | (NM_001256847, |
| chr17 | 915806 | rockermeth | rockermethSegs | −1 | 428 | 638 | intron | (NM_001256847, |
| chr17 | 915890 | rockermeth | rockermethSegs | −1 | 430 | 639 | intron | (NM_001256847, |
| chr17 | 915912 | rockermeth | rockermethSegs | −1 | 431 | 640 | intron | (NM_001256847, |
| chr17 | 7010397 | rockermeth | rockermethSegs | −1 | 135 | 641 | exon | (NM_001201352, |
| chr17 | 7010445 | rockermeth | rockermethSegs | −1 | 132 | 642 | exon | (NM_001201352, |
| chr17 | 7010504 | rockermeth | rockermethSegs | −1 | 138 | 643 | intron | (NM_001181, |
| chr17 | 7010533 | rockermeth | rockermethSegs | −1 | 136 | 644 | intron | (NM_001181, |
| chr17 | 7010548 | rockermeth | rockermethSegs | −1 | 134 | 645 | exon | (NM_001201352, |
| chr17 | 7010630 | rockermeth | rockermethSegs | −1 | 137 | 646 | exon | (NM_001201352, |
| chr17 | 7010663 | rockermeth | rockermethSegs | −1 | 133 | 647 | intron | (NM_001181, |
| chr17 | 10728383 | rockermeth | rockermethSegs | −1 | 425 | 648 | 3′ | UTR |
| chr17 | 10740512 | rockermeth | rockermethSegs | −1 | 427 | 649 | intron | (NM_001101387, |
| chr17 | 10740558 | rockermeth | rockermethSegs | −1 | 422 | 650 | intron | (NM_001101387, |
| chr17 | 10740569 | rockermeth | rockermethSegs | −1 | 423 | 651 | intron | (NM_001101387, |
| chr17 | 10740581 | rockermeth | rockermethSegs | −1 | 424 | 652 | intron | (NM_001101387, |
| chr17 | 10740595 | rockermeth | rockermethSegs | −1 | 426 | 653 | intron | (NM_001101387, |
| chr17 | 17677711 | rockermeth | rockermethSegs | 1 | 89 | 654 | intron | (NM_030665, |
| chr17 | 17677757 | rockermeth | rockermethSegs | 1 | 88 | 655 | intron | (NM_030665, |
| chr17 | 17677887 | rockermeth | rockermethSegs | 1 | 90 | 656 | intron | (NM_030665, |
| chr17 | 21286388 | rockermeth | rockermethSegs | −1 | 599 | 657 | intron | (NM_021012, |
| chr17 | 21286444 | rockermeth | rockermethSegs | −1 | 592 | 658 | intron | (NM_021012, |
| chr17 | 21286486 | rockermeth | rockermethSegs | −1 | 598 | 659 | intron | (NM_021012, |
| chr17 | 21286510 | rockermeth | rockermethSegs | −1 | 593 | 660 | intron | (NM_021012, |
| chr17 | 21286534 | rockermeth | rockermethSegs | −1 | 594 | 661 | intron | (NM_021012, |
| chr17 | 21286708 | rockermeth | rockermethSegs | −1 | 595 | 662 | intron | (NM_021012, |
| chr17 | 21286741 | rockermeth | rockermethSegs | −1 | 596 | 663 | intron | (NM_021012, |
| chr17 | 21286747 | rockermeth | rockermethSegs | −1 | 597 | 664 | intron | (NM_021012, |
| chr17 | 28299552 | rockermeth | rockermethSegs | −1 | 45 | 665 | intron | (NR_026738, |
| chr17 | 28299565 | rockermeth | rockermethSegs | −1 | 44 | 666 | intron | (NR_026738, |
| chr17 | 28299571 | rockermeth | rockermethSegs | −1 | 42 | 667 | intron | (NR_026738, |
| chr17 | 28299598 | rockermeth | rockermethSegs | −1 | 43 | 668 | intron | (NR_026738, |
| chr17 | 28299697 | rockermeth | rockermethSegs | −1 | 41 | 669 | intron | (NR_026738, |
| chr17 | 71715903 | rockermeth | rockermethSegs | −1 | 762 | 670 | Intergenic | |
| chr17 | 71715940 | rockermeth | rockermethSegs | −1 | 761 | 671 | Intergenic | |
| chr17 | 71715947 | rockermeth | rockermethSegs | −1 | 760 | 672 | Intergenic | |
| chr17 | 71715958 | rockermeth | rockermethSegs | −1 | 758 | 673 | Intergenic | |
| chr17 | 71716005 | rockermeth | rockermethSegs | −1 | 759 | 674 | Intergenic | |
| chr17 | 71743873 | rockermeth | rockermethSegs | −1 | 224 | 675 | intron | (NM_001278587, |
| chr17 | 71744668 | rockermeth | rockermethSegs | −1 | 220 | 676 | TTS | (NR_027146) |
| chr17 | 71744685 | rockermeth | rockermethSegs | −1 | 223 | 677 | TTS | (NR_027146) |
| chr17 | 71744723 | rockermeth | rockermethSegs | −1 | 221 | 678 | TTS | (NR_027146) |
| chr17 | 71744739 | rockermeth | rockermethSegs | −1 | 222 | 679 | TTS | (NR_027146) |
| chr17 | 78079606 | rockermeth | rockermethSegs | −1 | 607 | 680 | exon | (NM_000152, |
| chr17 | 78079613 | rockermeth | rockermethSegs | −1 | 610 | 681 | exon | (NM_000152, |
| chr17 | 78079629 | rockermeth | rockermethSegs | −1 | 608 | 682 | exon | (NM_000152, |
| chr17 | 78079641 | rockermeth | rockermethSegs | −1 | 609 | 683 | exon | (NM_000152, |
| chr17 | 78079653 | rockermeth | rockermethSegs | −1 | 611 | 684 | exon | (NM_000152, |
| chr17 | 78395359 | rockermeth | rockermethSegs | −1 | 722 | 685 | intron | (NR_148041, |
| chr17 | 78395376 | rockermeth | rockermethSegs | −1 | 723 | 686 | intron | (NR_148041, |
| chr17 | 80314127 | rockermeth | rockermethSegs | 1 | 8 | 687 | Intergenic | |
| chr17 | 80314154 | rockermeth | rockermethSegs | 1 | 9 | 688 | Intergenic | |
| chr17 | 80914812 | rockermeth | rockermethSegs | −1 | 626 | 689 | intron | (NR_135465, |
| chr17 | 80915007 | rockermeth | rockermethSegs | −1 | 632 | 690 | intron | (NR_135465, |
| chr17 | 80915042 | rockermeth | rockermethSegs | −1 | 633 | 691 | intron | (NR_135465, |
| chr17 | 80915160 | rockermeth | rockermethSegs | −1 | 628 | 692 | intron | (NR_135465, |
| chr17 | 80915190 | rockermeth | rockermethSegs | −1 | 634 | 693 | intron | (NR_135465, |
| chr17 | 80915238 | rockermeth | rockermethSegs | −1 | 627 | 694 | exon | (NM_001009905, |
| chr17 | 80915248 | rockermeth | rockermethSegs | −1 | 630 | 695 | exon | (NM_001009905, |
| chr17 | 80915270 | rockermeth | rockermethSegs | −1 | 631 | 696 | exon | (NM_001009905, |
| chr17 | 80915289 | rockermeth | rockermethSegs | −1 | 629 | 697 | exon | (NM_001009905, |
| chr17 | 80915308 | rockermeth | rockermethSegs | −1 | 625 | 698 | exon | (NM_001009905, |
| chr18 | 8659649 | rockermeth | rockermethSegs | −1 | 75 | 699 | Intergenic | |
| chr18 | 24348426 | rockermeth | rockermethSegs | −1 | 622 | 700 | Intergenic | |
| chr18 | 25366256 | rockermeth | rockermethSegs | −1 | 215 | 701 | Intergenic | |
| chr18 | 25366262 | rockermeth | rockermethSegs | −1 | 209 | 702 | Intergenic | |
| chr18 | 25366300 | rockermeth | rockermethSegs | −1 | 212 | 703 | Intergenic | |
| chr18 | 25366309 | rockermeth | rockermethSegs | −1 | 214 | 704 | Intergenic | |
| chr18 | 25442175 | rockermeth | rockermethSegs | −1 | 213 | 705 | Intergenic | |
| chr18 | 25442224 | rockermeth | rockermethSegs | −1 | 210 | 706 | Intergenic | |
| chr18 | 25442243 | rockermeth | rockermethSegs | −1 | 211 | 707 | Intergenic | |
| chr18 | 68166868 | rockermeth | rockermethSegs | −1 | 77 | 708 | Intergenic | |
| chr18 | 68166878 | rockermeth | rockermethSegs | −1 | 76 | 709 | Intergenic | |
| chr18 | 77320496 | rockermeth | rockermethSegs | −1 | 475 | 710 | Intergenic | |
| chr18 | 77320538 | rockermeth | rockermethSegs | −1 | 471 | 711 | Intergenic | |
| chr18 | 77320583 | rockermeth | rockermethSegs | −1 | 476 | 712 | Intergenic | |
| chr18 | 77320593 | rockermeth | rockermethSegs | −1 | 473 | 713 | Intergenic | |
| chr18 | 77320606 | rockermeth | rockermethSegs | −1 | 474 | 714 | Intergenic | |
| chr18 | 77320672 | rockermeth | rockermethSegs | −1 | 477 | 715 | Intergenic | |
| chr18 | 77320695 | rockermeth | rockermethSegs | −1 | 478 | 716 | Intergenic | |
| chr18 | 77320711 | rockermeth | rockermethSegs | −1 | 472 | 717 | Intergenic | |
| chr18 | 77320739 | rockermeth | rockermethSegs | −1 | 479 | 718 | Intergenic | |
| chr19 | 883734 | rockermeth | rockermethSegs | −1 | 368 | 719 | intron | (NM_005481, |
| chr19 | 883775 | rockermeth | rockermethSegs | −1 | 377 | 720 | intron | (NM_005481, |
| chr19 | 883830 | rockermeth | rockermethSegs | −1 | 367 | 721 | intron | (NM_005481, |
| chr19 | 883873 | rockermeth | rockermethSegs | −1 | 365 | 722 | intron | (NM_005481, |
| chr19 | 883885 | rockermeth | rockermethSegs | −1 | 373 | 723 | intron | (NM_005481, |
| chr19 | 883994 | rockermeth | rockermethSegs | −1 | 370 | 724 | intron | (NM_005481, |
| chr19 | 884032 | rockermeth | rockermethSegs | −1 | 374 | 725 | intron | (NM_005481, |
| chr19 | 884042 | rockermeth | rockermethSegs | −1 | 369 | 726 | intron | (NM_005481, |
| chr19 | 884057 | rockermeth | rockermethSegs | −1 | 375 | 727 | intron | (NM_005481, |
| chr19 | 884140 | rockermeth | rockermethSegs | −1 | 364 | 728 | intron | (NM_005481, |
| chr19 | 884147 | rockermeth | rockermethSegs | −1 | 371 | 729 | intron | (NM_005481, |
| chr19 | 884172 | rockermeth | rockermethSegs | −1 | 372 | 730 | intron | (NM_005481, |
| chr19 | 884233 | rockermeth | rockermethSegs | −1 | 376 | 731 | intron | (NM_005481, |
| chr19 | 884267 | rockermeth | rockermethSegs | −1 | 363 | 732 | intron | (NM_005481, |
| chr19 | 884346 | rockermeth | rockermethSegs | −1 | 366 | 733 | intron | (NM_005481, |
| chr19 | 2299058 | rockermeth | rockermethSegs | −1 | 82 | 734 | intron | (NM_001101391, |
| chr19 | 2299180 | rockermeth | rockermethSegs | −1 | 78 | 735 | intron | (NM_001101391, |
| chr19 | 2299218 | rockermeth | rockermethSegs | −1 | 81 | 736 | intron | (NM_001101391, |
| chr19 | 2299353 | rockermeth | rockermethSegs | −1 | 79 | 737 | intron | (NM_001101391, |
| chr19 | 2299390 | rockermeth | rockermethSegs | −1 | 80 | 738 | intron | (NM_001101391, |
| chr19 | 4566603 | rockermeth | rockermethSegs | −1 | 49 | 739 | Intergenic | |
| chr19 | 4566636 | rockermeth | rockermethSegs | −1 | 46 | 740 | Intergenic | |
| chr19 | 4566642 | rockermeth | rockermethSegs | −1 | 47 | 741 | Intergenic | |
| chr19 | 4566653 | rockermeth | rockermethSegs | −1 | 48 | 742 | Intergenic | |
| chr19 | 9994121 | rockermeth | rockermethSegs | −1 | 149 | 743 | intron | (NM_058164, |
| chr19 | 9994137 | rockermeth | rockermethSegs | −1 | 145 | 744 | intron | (NM_058164, |
| chr19 | 9994208 | rockermeth | rockermethSegs | −1 | 146 | 745 | intron | (NM_058164, |
| chr19 | 9994219 | rockermeth | rockermethSegs | −1 | 147 | 746 | intron | (NM_058164, |
| chr19 | 9994333 | rockermeth | rockermethSegs | −1 | 148 | 747 | intron | (NM_058164, |
| chr19 | 9994349 | rockermeth | rockermethSegs | −1 | 150 | 748 | intron | (NM_058164, |
| chr19 | 13108021 | rockermeth | rockermethSegs | 1 | 104 | 749 | intron | (NM_001365982, |
| chr19 | 13108640 | rockermeth | rockermethSegs | 1 | 105 | 750 | intron | (NM_001365982, |
| chr19 | 13112778 | rockermeth | rockermethSegs | 1 | 103 | 751 | intron | (NM_001365982, |
| chr19 | 13616660 | rockermeth | rockermethSegs | 1 | 56 | 752 | intron | (NM_001127222, |
| chr19 | 13616810 | rockermeth | rockermethSegs | 1 | 38 | 753 | exon | (NM_001127221, |
| chr19 | 13616818 | rockermeth | rockermethSegs | 1 | 24 | 754 | exon | (NM_001127221, |
| chr19 | 13616834 | rockermeth | rockermethSegs | 1 | 34 | 755 | exon | (NM_001127221, |
| chr19 | 13616868 | rockermeth | rockermethSegs | 1 | 39 | 756 | exon | (NM_001127221, |
| chr19 | 13616879 | rockermeth | rockermethSegs | 1 | 57 | 757 | exon | (NM_001127221, |
| chr19 | 13616906 | rockermeth | rockermethSegs | 1 | 61 | 758 | exon | (NM_001127221, |
| chr19 | 13616911 | rockermeth | rockermethSegs | 1 | 60 | 759 | exon | (NM_001127221, |
| chr19 | 13617010 | rockermeth | rockermethSegs | 1 | 45 | 760 | exon | (NM_001127221, |
| chr19 | 13617018 | rockermeth | rockermethSegs | 1 | 20 | 761 | exon | (NM_001127221, |
| chr19 | 13617024 | rockermeth | rockermethSegs | 1 | 43 | 762 | exon | (NM_001127221, |
| chr19 | 13617029 | rockermeth | rockermethSegs | 1 | 28 | 763 | exon | (NM_001127221, |
| chr19 | 13617058 | rockermeth | rockermethSegs | 1 | 47 | 764 | 5′ | UTR |
| chr19 | 13617065 | rockermeth | rockermethSegs | 1 | 48 | 765 | 5′ | UTR |
| chr19 | 13617070 | rockermeth | rockermethSegs | 1 | 40 | 766 | 5′ | UTR |
| chr19 | 13617082 | rockermeth | rockermethSegs | 1 | 27 | 767 | 5′ | UTR |
| chr19 | 13617090 | rockermeth | rockermethSegs | 1 | 25 | 768 | 5′ | UTR |
| chr19 | 13617128 | rockermeth | rockermethSegs | 1 | 51 | 769 | 5′ | UTR |
| chr19 | 13617140 | rockermeth | rockermethSegs | 1 | 33 | 770 | 5′ | UTR |
| chr19 | 13617157 | rockermeth | rockermethSegs | 1 | 29 | 771 | 5′ | UTR |
| chr19 | 13617163 | rockermeth | rockermethSegs | 1 | 30 | 772 | 5′ | UTR |
| chr19 | 13617169 | rockermeth | rockermethSegs | 1 | 41 | 773 | 5′ | UTR |
| chr19 | 13617176 | rockermeth | rockermethSegs | 1 | 21 | 774 | promoter-TSS | (NM_001127221) |
| chr19 | 13617194 | rockermeth | rockermethSegs | 1 | 22 | 775 | promoter-TSS | (NM_001127221) |
| chr19 | 13617208 | rockermeth | rockermethSegs | 1 | 26 | 776 | promoter-TSS | (NM_001127221) |
| chr19 | 13617218 | rockermeth | rockermethSegs | 1 | 31 | 777 | promoter-TSS | (NM_001127221) |
| chr19 | 13617233 | rockermeth | rockermethSegs | 1 | 50 | 778 | promoter-TSS | (NM_001127221) |
| chr19 | 13617241 | rockermeth | rockermethSegs | 1 | 18 | 779 | promoter-TSS | (NM_001127221) |
| chr19 | 13617250 | rockermeth | rockermethSegs | 1 | 55 | 780 | promoter-TSS | (NM_001127221) |
| chr19 | 13617265 | rockermeth | rockermethSegs | 1 | 37 | 781 | promoter-TSS | (NM_001127221) |
| chr19 | 13617288 | rockermeth | rockermethSegs | 1 | 42 | 782 | promoter-TSS | (NM_001127221) |
| chr19 | 13617302 | rockermeth | rockermethSegs | 1 | 53 | 783 | promoter-TSS | (NM_001127221) |
| chr19 | 13617315 | rockermeth | rockermethSegs | 1 | 58 | 784 | promoter-TSS | (NM_001127221) |
| chr19 | 13617321 | rockermeth | rockermethSegs | 1 | 54 | 785 | promoter-TSS | (NM_001127221) |
| chr19 | 13617328 | rockermeth | rockermethSegs | 1 | 46 | 786 | promoter-TSS | (NM_001127221) |
| chr19 | 13617336 | rockermeth | rockermethSegs | 1 | 62 | 787 | promoter-TSS | (NM_001127221) |
| chr19 | 13617364 | rockermeth | rockermethSegs | 1 | 23 | 788 | promoter-TSS | (NM_001127221) |
| chr19 | 13617380 | rockermeth | rockermethSegs | 1 | 17 | 789 | promoter-TSS | (NM_001127221) |
| chr19 | 13617419 | rockermeth | rockermethSegs | 1 | 19 | 790 | promoter-TSS | (NM_001127221) |
| chr19 | 13617465 | rockermeth | rockermethSegs | 1 | 35 | 791 | promoter-TSS | (NM_001127221) |
| chr19 | 13617483 | rockermeth | rockermethSegs | 1 | 32 | 792 | promoter-TSS | (NM_001127221) |
| chr19 | 13617503 | rockermeth | rockermethSegs | 1 | 15 | 793 | promoter-TSS | (NM_001127221) |
| chr19 | 13617512 | rockermeth | rockermethSegs | 1 | 52 | 794 | promoter-TSS | (NM_001127221) |
| chr19 | 13617517 | rockermeth | rockermethSegs | 1 | 16 | 795 | promoter-TSS | (NM_001127221) |
| chr19 | 13617541 | rockermeth | rockermethSegs | 1 | 44 | 796 | promoter-TSS | (NM_001127221) |
| chr19 | 13618356 | rockermeth | rockermethSegs | 1 | 36 | 797 | Intergenic | |
| chr19 | 13618391 | rockermeth | rockermethSegs | 1 | 49 | 798 | Intergenic | |
| chr19 | 13700379 | rockermeth | rockermethSegs | 1 | 59 | 799 | Intergenic | |
| chr19 | 18836689 | rockermeth | rockermethSegs | −1 | 4 | 800 | intron | (NM_001098482, |
| chr19 | 18837043 | rockermeth | rockermethSegs | −1 | 7 | 801 | intron | (NM_001098482, |
| chr19 | 18837091 | rockermeth | rockermethSegs | −1 | 6 | 802 | intron | (NM_001098482, |
| chr19 | 18837098 | rockermeth | rockermethSegs | −1 | 5 | 803 | intron | (NM_001098482, |
| chr19 | 18837105 | rockermeth | rockermethSegs | −1 | 8 | 804 | intron | (NM_001098482, |
| chr19 | 34181556 | rockermeth | rockermethSegs | −1 | 419 | 805 | intron | (NM_001127895, |
| chr19 | 34181606 | rockermeth | rockermethSegs | −1 | 421 | 806 | intron | (NM_001127895, |
| chr19 | 34181636 | rockermeth | rockermethSegs | −1 | 420 | 807 | intron | (NM_001127895, |
| chr19 | 38527887 | rockermeth | rockermethSegs | −1 | 71 | 808 | intron | (NM_015073, |
| chr19 | 38527913 | rockermeth | rockermethSegs | −1 | 72 | 809 | intron | (NM_015073, |
| chr19 | 38527932 | rockermeth | rockermethSegs | −1 | 73 | 810 | intron | (NM_015073, |
| chr19 | 38528016 | rockermeth | rockermethSegs | −1 | 74 | 811 | intron | (NM_015073, |
| chr19 | 38528031 | rockermeth | rockermethSegs | −1 | 70 | 812 | intron | (NM_015073, |
| chr19 | 38684235 | rockermeth | rockermethSegs | −1 | 62 | 813 | exon | (NM_015073, |
| chr19 | 38684327 | rockermeth | rockermethSegs | −1 | 63 | 814 | exon | (NM_015073, |
| chr19 | 38684338 | rockermeth | rockermethSegs | −1 | 61 | 815 | exon | (NM_015073, |
| chr19 | 38684363 | rockermeth | rockermethSegs | −1 | 60 | 816 | exon | (NM_015073, |
| chr19 | 48699558 | rockermeth | rockermethSegs | −1 | 159 | 817 | exon | (NM_199341, |
| chr19 | 48699578 | rockermeth | rockermethSegs | −1 | 158 | 818 | exon | (NM_199341, |
| chr19 | 48699588 | rockermeth | rockermethSegs | −1 | 157 | 819 | exon | (NM_199341, |
| chr19 | 48699615 | rockermeth | rockermethSegs | −1 | 161 | 820 | exon | (NM_199341, |
| chr19 | 48699628 | rockermeth | rockermethSegs | −1 | 156 | 821 | exon | (NM_199341, |
| chr19 | 48699667 | rockermeth | rockermethSegs | −1 | 155 | 822 | exon | (NM_199341, |
| chr19 | 48699717 | rockermeth | rockermethSegs | −1 | 160 | 823 | exon | (NM_199341, |
| chr19 | 55870392 | rockermeth | rockermethSegs | −1 | 619 | 824 | exon | (NM_001145402, |
| chr19 | 55870402 | rockermeth | rockermethSegs | −1 | 620 | 825 | exon | (NM_001145402, |
| chr19 | 55870407 | rockermeth | rockermethSegs | −1 | 615 | 826 | exon | (NM_001145402, |
| chr19 | 55870425 | rockermeth | rockermethSegs | −1 | 618 | 827 | exon | (NM_001145402, |
| chr19 | 55870439 | rockermeth | rockermethSegs | −1 | 613 | 828 | exon | (NM_001145402, |
| chr19 | 55870456 | rockermeth | rockermethSegs | −1 | 616 | 829 | exon | (NM_001145402, |
| chr19 | 55870462 | rockermeth | rockermethSegs | −1 | 614 | 830 | exon | (NM_001145402, |
| chr19 | 55870471 | rockermeth | rockermethSegs | −1 | 617 | 831 | exon | (NM_001145402, |
| chr19 | 55870886 | rockermeth | rockermethSegs | −1 | 621 | 832 | exon | (NM_001145402, |
| chr20 | 20180688 | rockermeth | rockermethSegs | −1 | 414 | 833 | intron | (NM_015585, |
| chr20 | 20180732 | rockermeth | rockermethSegs | −1 | 413 | 834 | intron | (NM_015585, |
| chr20 | 20188098 | rockermeth | rockermethSegs | −1 | 409 | 835 | intron | (NM_015585, |
| chr20 | 20188151 | rockermeth | rockermethSegs | −1 | 411 | 836 | intron | (NM_015585, |
| chr20 | 20206077 | rockermeth | rockermethSegs | −1 | 416 | 837 | intron | (NM_015585, |
| chr20 | 20232312 | rockermeth | rockermethSegs | −1 | 417 | 838 | exon | (NM_015585, |
| chr20 | 20232336 | rockermeth | rockermethSegs | −1 | 412 | 839 | exon | (NM_015585, |
| chr20 | 20232350 | rockermeth | rockermethSegs | −1 | 408 | 840 | exon | (NM_015585, |
| chr20 | 20232360 | rockermeth | rockermethSegs | −1 | 410 | 841 | exon | (NM_015585, |
| chr20 | 20232370 | rockermeth | rockermethSegs | −1 | 415 | 842 | exon | (NM_015585, |
| chr20 | 20232391 | rockermeth | rockermethSegs | −1 | 418 | 843 | exon | (NM_015585, |
| chr20 | 20376947 | rockermeth | rockermethSegs | −1 | 252 | 844 | intron | (NM_020343, |
| chr20 | 20376962 | rockermeth | rockermethSegs | −1 | 251 | 845 | intron | (NM_020343, |
| chr20 | 20377074 | rockermeth | rockermethSegs | −1 | 239 | 846 | intron | (NM_020343, |
| chr20 | 20377089 | rockermeth | rockermethSegs | −1 | 249 | 847 | intron | (NM_020343, |
| chr20 | 20377110 | rockermeth | rockermethSegs | −1 | 244 | 848 | intron | (NM_020343, |
| chr20 | 20377122 | rockermeth | rockermethSegs | −1 | 247 | 849 | intron | (NM_020343, |
| chr20 | 20377130 | rockermeth | rockermethSegs | −1 | 238 | 850 | intron | (NM_020343, |
| chr20 | 20377162 | rockermeth | rockermethSegs | −1 | 248 | 851 | intron | (NM_020343, |
| chr20 | 20377169 | rockermeth | rockermethSegs | −1 | 246 | 852 | intron | (NM_020343, |
| chr20 | 20377206 | rockermeth | rockermethSegs | −1 | 250 | 853 | intron | (NM_020343, |
| chr20 | 20392139 | rockermeth | rockermethSegs | −1 | 245 | 854 | intron | (NM_020343, |
| chr20 | 20392184 | rockermeth | rockermethSegs | −1 | 235 | 855 | intron | (NM_020343, |
| chr20 | 20392215 | rockermeth | rockermethSegs | −1 | 236 | 856 | intron | (NM_020343, |
| chr20 | 20392408 | rockermeth | rockermethSegs | −1 | 237 | 857 | intron | (NM_020343, |
| chr20 | 20421295 | rockermeth | rockermethSegs | −1 | 253 | 858 | intron | (NM_020343, |
| chr20 | 20421314 | rockermeth | rockermethSegs | −1 | 254 | 859 | intron | (NM_020343, |
| chr20 | 20421345 | rockermeth | rockermethSegs | −1 | 240 | 860 | intron | (NM_020343, |
| chr20 | 20421371 | rockermeth | rockermethSegs | −1 | 243 | 861 | intron | (NM_020343, |
| chr20 | 20421396 | rockermeth | rockermethSegs | −1 | 241 | 862 | intron | (NM_020343, |
| chr20 | 20421420 | rockermeth | rockermethSegs | −1 | 242 | 863 | intron | (NM_020343, |
| chr20 | 20421459 | rockermeth | rockermethSegs | −1 | 255 | 864 | intron | (NM_020343, |
| chr20 | 20421486 | rockermeth | rockermethSegs | −1 | 256 | 865 | intron | (NM_020343, |
| chr20 | 31129481 | rockermeth | rockermethSegs | −1 | 261 | 866 | intron | (NM_001256798, |
| chr20 | 36923352 | rockermeth | rockermethSegs | −1 | 142 | 867 | Intergenic | |
| chr20 | 36923369 | rockermeth | rockermethSegs | −1 | 140 | 868 | Intergenic | |
| chr20 | 36923397 | rockermeth | rockermethSegs | −1 | 141 | 869 | Intergenic | |
| chr20 | 61013054 | rockermeth | rockermethSegs | −1 | 445 | 870 | intron | (NR_033263, |
| chr20 | 61013094 | rockermeth | rockermethSegs | −1 | 440 | 871 | intron | (NR_033263, |
| chr20 | 61013113 | rockermeth | rockermethSegs | −1 | 444 | 872 | intron | (NR_033263, |
| chr20 | 61013148 | rockermeth | rockermethSegs | −1 | 437 | 873 | intron | (NR_033263, |
| chr20 | 61013167 | rockermeth | rockermethSegs | −1 | 434 | 874 | intron | (NR_033263, |
| chr20 | 61013187 | rockermeth | rockermethSegs | −1 | 433 | 875 | intron | (NR_033263, |
| chr20 | 61013226 | rockermeth | rockermethSegs | −1 | 436 | 876 | intron | (NR_033263, |
| chr20 | 61013242 | rockermeth | rockermethSegs | −1 | 438 | 877 | intron | (NR_033263, |
| chr20 | 61013251 | rockermeth | rockermethSegs | −1 | 435 | 878 | intron | (NR_033263, |
| chr20 | 61013263 | rockermeth | rockermethSegs | −1 | 447 | 879 | intron | (NR_033263, |
| chr20 | 61013303 | rockermeth | rockermethSegs | −1 | 448 | 880 | intron | (NR_033263, |
| chr20 | 61013339 | rockermeth | rockermethSegs | −1 | 442 | 881 | intron | (NR_033263, |
| chr20 | 61013474 | rockermeth | rockermethSegs | −1 | 446 | 882 | intron | (NR_033263, |
| chr20 | 61013515 | rockermeth | rockermethSegs | −1 | 441 | 883 | intron | (NR_033263, |
| chr20 | 61020520 | rockermeth | rockermethSegs | −1 | 439 | 884 | intron | (NR_033263, |
| chr20 | 61021747 | rockermeth | rockermethSegs | −1 | 443 | 885 | intron | (NR_033263, |
| chr21 | 43442046 | rockermeth | rockermethSegs | −1 | 574 | 886 | promoter-TSS | (NR_119385) |
| chr21 | 43442055 | rockermeth | rockermethSegs | −1 | 573 | 887 | promoter-TSS | (NR_119385) |
| chr21 | 43442083 | rockermeth | rockermethSegs | −1 | 575 | 888 | promoter-TSS | (NR_119385) |
| chr21 | 43442252 | rockermeth | rockermethSegs | −1 | 576 | 889 | promoter-TSS | (NR_027273) |
| chr21 | 43442338 | rockermeth | rockermethSegs | −1 | 572 | 890 | promoter-TSS | (NR_027273) |
| chr21 | 43442384 | rockermeth | rockermethSegs | −1 | 577 | 891 | promoter-TSS | (NR_027273) |
| chr21 | 44836763 | rockermeth | rockermethSegs | −1 | 492 | 892 | exon | (NM_001320643, |
| chr21 | 44836797 | rockermeth | rockermethSegs | −1 | 489 | 893 | exon | (NM_001320643, |
| chr21 | 144836823 | rockermeth | rockermethSegs | −1 | 493 | 894 | exon | (NM_001320643, |
| chr21 | 44836842 | rockermeth | rockermethSegs | −1 | 490 | 895 | exon | (NM_001320643, |
| chr21 | 44836924 | rockermeth | rockermethSegs | −1 | 484 | 896 | exon | (NM_001320643, |
| chr21 | 44836936 | rockermeth | rockermethSegs | −1 | 487 | 897 | exon | (NM_001320643, |
| chr21 | 44836971 | rockermeth | rockermethSegs | −1 | 480 | 898 | exon | (NM_001320643, |
| chr21 | 44837026 | rockermeth | rockermethSegs | −1 | 481 | 899 | intron | (NM_173354, |
| chr21 | 44837044 | rockermeth | rockermethSegs | −1 | 482 | 900 | intron | (NM_173354, |
| chr21 | 44837052 | rockermeth | rockermethSegs | −1 | 488 | 901 | intron | (NM_173354, |
| chr21 | 44837060 | rockermeth | rockermethSegs | −1 | 485 | 902 | intron | (NM_173354, |
| chr21 | 44837074 | rockermeth | rockermethSegs | −1 | 486 | 903 | intron | (NM_173354, |
| chr21 | 44837115 | rockermeth | rockermethSegs | −1 | 483 | 904 | intron | (NM_173354, |
| chr21 | 44837132 | rockermeth | rockermethSegs | −1 | 491 | 905 | intron | (NM_173354, |
| chr21 | 44837157 | rockermeth | rockermethSegs | −1 | 494 | 906 | intron | (NM_173354, |
| chr21 | 145653981 | rockermeth | rockermethSegs | −1 | 233 | 907 | intron | (NM_015259, |
| chr21 | 45654156 | rockermeth | rockermethSegs | −1 | 234 | 908 | intron | (NM_015259, |
| chr22 | 47444997 | rockermeth | rockermethSegs | −1 | 727 | 909 | intron | (NM_014346, |
| chr22 | 47445037 | rockermeth | rockermethSegs | −1 | 724 | 910 | intron | (NM_014346, |
| chr22 | 47447406 | rockermeth | rockermethSegs | −1 | 726 | 911 | intron | (NM_014346, |
| chr22 | 47447603 | rockermeth | rockermethSegs | −1 | 725 | 912 | intron | (NM_014346, |
| chrX | 104458760 | rockermeth | rockermethSegs | −1 | 765 | 913 | intron | (NM_017416, |
| chrX | 104692650 | rockermeth | rockermethSegs | −1 | 766 | 914 | intron | (NM_017416, |
| chrX | 104804571 | rockermeth | rockermethSegs | −1 | 764 | 915 | intron | (NM_017416, |
| chrX | 104891135 | rockermeth | rockermethSegs | −1 | 763 | 916 | intron | (NM_017416, |
| chrX | 105016634 | rockermeth | rockermethSegs | −1 | 767 | 917 | Intergenic | |
| chrX | 105016672 | rockermeth | rockermethSegs | −1 | 768 | 918 | Intergenic | |
| chrX | 128926432 | rockermeth | rockermethSegs | −1 | 526 | 919 | exon | (NM_018990, |
| chr2 | 209103955 | epi_snvs | snv_frequency | | 1 | 1 | intron | (NM_005896, |
| chr4 | 106155150 | epi_snvs | snv_frequency | | 1 | 2 | exon | (NM_001127208, |
| chr4 | 106155913 | epi_snvs | snv_frequency | | 1 | 3 | exon | (NM_001127208, |
| chr4 | 106156460 | epi_snvs | snv_frequency | | 1 | 4 | exon | (NM_001127208, |
| chr4 | 106156742 | epi_snvs | snv_frequency | | 1 | 5 | exon | (NM_001127208, |
| chr4 | 106157415 | epi_snvs | snv_frequency | | 1 | 6 | exon | (NM_001127208, |
| chr4 | 106164901 | epi_snvs | snv_frequency | | 1 | 7 | TTS | (NM_017628) |
| chr4 | 106196236 | epi_snvs | snv_frequency | | 1 | 8 | exon | (NM_001127208, |
| chr7 | 33732332 | AUC_Δβ_GOI | snv_frequency | | 1 | 1 | Intergenic | |
| chr7 | 140453119 | epi_snvs | snv_frequency | | 1 | 9 | exon | (NM_004333, |
| chr7 | 140481387 | epi_snvs | snv_frequency | | 1 | 10 | exon | (NM_004333, |
| chr7 | 140481426 | epi_snvs | snv_frequency | | 1 | 11 | exon | (NM_004333, |
| chr7 | 140500421 | epi_snvs | snv_frequency | | 1 | 12 | intron | (NR_148928, |
| chr7 | 140507748 | epi_snvs | snv_frequency | | 1 | 13 | exon | (NM_004333, |
| chr7 | 140507954 | epi_snvs | snv_frequency | | 1 | 14 | intron | (NM_001354609, |
| chr7 | 140624441 | epi_snvs | snv_frequency | | 1 | 15 | exon | (NM_004333, |
| chr14 | 38061176 | FOXA1_snvs | snv_frequency | | 1 | 1 | exon | (NM_004496, |
| chr14 | 38061319 | FOXA1_snvs | snv_frequency | | 1 | 2 | exon | (NM_004496, |
| chr14 | 38061830 | FOXA1_snvs | snv_frequency | | 1 | 3 | exon | (NM_004496, |
| chr17 | 7572948 | TP53_snvs | snv_frequency | | 1 | 1 | exon | (NM_000546, |
| chr17 | 7573967 | TP53_snvs | snv_frequency | | 1 | 2 | exon | (NM_000546, |
| chr17 | 7576524 | TP53_snvs | snv_frequency | | 1 | 3 | exon | (NM_001126117, |
| chr17 | 7576838 | TP53_snvs | snv_frequency | | 1 | 4 | exon | (NM_001126117, |
| chr17 | 7577020 | TP53_snvs | snv_frequency | | 1 | 5 | exon | (NM_001126117, |
| chr17 | 7577483 | TP53_snvs | snv_frequency | | 1 | 6 | exon | (NM_001126117, |
| chr17 | 7578161 | TP53_snvs | snv_frequency | | 1 | 7 | exon | (NM_001126117, |
| chr17 | 7578373 | TP53_snvs | snv_frequency | | 1 | 8 | promoter-TSS | (NM_001276697) |
| chr17 | 7579297 | TP53_snvs | snv_frequency | | 1 | 9 | promoter-TSS | (NM_001126117) |
| chr17 | 7579393 | TP53_snvs | snv_frequency | | 1 | 10 | promoter-TSS | (NM_001126117) |
| chr17 | 7579506 | TP53_snvs | snv_frequency | | 1 | 11 | promoter-TSS | (NM_001126117) |
| chr17 | 7579701 | TP53_snvs | snv_frequency | | 1 | 12 | promoter-TSS | (NM_001126117) |
| chr17 | 7579846 | TP53_snvs | snv_frequency | | 1 | 13 | exon | (NM_000546, |
| chr17 | 47696409 | SPOP_snvs | snv_frequency | | 1 | 1 | exon | (NM_001007228, |
| chr17 | 47696673 | SPOP_snvs | snv_frequency | | 1 | 2 | exon | (NM_001007228, |
| chr20 | 31375142 | epi_snvs | snv_frequency | | 1 | 16 | exon | (NM_175848, |
| chr20 | 31380384 | epi_snvs | snv_frequency | | 1 | 17 | intron | (NM_175849, |
| chr20 | 31381300 | epi_snvs | snv_frequency | | 1 | 18 | intron | (NM_175849, |
| chr20 | 31386255 | epi_snvs | snv_frequency | | 1 | 19 | exon | (NM_175848, |
| chr20 | 31388050 | epi_snvs | snv_frequency | | 1 | 20 | exon | (NM_175848, |
| chr20 | 31389120 | epi_snvs | snv_frequency | | 1 | 21 | exon | (NM_175848, |
| chr20 | 31393185 | epi_snvs | snv_frequency | | 1 | 22 | exon | (NM_175848, |
| chr20 | 31395662 | epi_snvs | snv_frequency | | 1 | 23 | exon | (NM_175848, |
| chrX | 66121000 | AR_Enhancer | snv_frequency | | 1 | 1 | Intergenic | |
| chrX | 66766332 | AR_snvs | snv_frequency | | 1 | 1 | exon | (NM_001348064, |
| chrX | 66931448 | AR_snvs | snv_frequency | | 1 | 2 | exon | (NM_000044, |
| chrX | 66937357 | AR_snvs | snv_frequency | | 1 | 3 | exon | (NM_000044, |
| chrX | 66943528 | AR_snvs | snv_frequency | | 1 | 4 | exon | (NM_000044, |
| chr2 | 25452399 | DMS_GOI | AUC_Δβ_GOI | −1 | | 1 | non-coding | (NR_135490, |
| chr2 | 25452969 | DMS_GOI | AUC_Δβ_GOI | −1 | | 2 | non-coding | (NR_135490, |
| chr2 | 25453099 | DMS_GOI | AUC_Δβ_GOI | −1 | | 3 | non-coding | (NR_135490, |
| chr2 | 25453105 | DMS_GOI | AUC_Δβ_GOI | −1 | | 4 | non-coding | (NR_135490, |
| chr2 | 25453799 | DMS_GOI | AUC_Δβ_GOI | −1 | | 5 | non-coding | (NR_135490, |
| chr2 | 25453898 | DMS_GOI | AUC_Δβ_GOI | −1 | | 6 | non-coding | (NR_135490, |
| chr2 | 25453899 | DMS_GOI | AUC_Δβ_GOI | −1 | | 7 | non-coding | (NR_135490, |
| chr2 | 25453913 | DMS_GOI | AUC_Δβ_GOI | −1 | | 8 | non-coding | (NR_135490, |
| chr2 | 25499982 | DMS_GOI | AUC_Δβ_GOI | 1 | | 9 | intron | (NR_135490, |
| chr2 | 25500271 | DMS_GOI | AUC_Δβ_GOI | 1 | | 10 | intron | (NR_135490, |
| chr2 | 25500272 | DMS_GOI | AUC_Δβ_GOI | 1 | | 11 | intron | (NR_135490, |
| chr2 | 239147428 | DMS_GOI | AUC_Δβ_GOI | −1 | | 12 | 3′ | UTR |
| chr2 | 239147440 | DMS_GOI | AUC_Δβ_GOI | −1 | | 13 | 3′ | UTR |
| chr2 | 239147441 | DMS_GOI | AUC_Δβ_GOI | −1 | | 14 | 3′ | UTR |
| chr2 | 239147485 | DMS_GOI | AUC_Δβ_GOI | −1 | | 15 | exon | (NM_018645, |
| chr2 | 239147486 | DMS_GOI | AUC_Δβ_GOI | −1 | | 16 | exon | (NM_018645, |
| chr2 | 239147522 | DMS_GOI | AUC_Δβ_GOI | −1 | | 17 | exon | (NM_018645, |
| chr2 | 239147534 | DMS_GOI | AUC_Δβ_GOI | −1 | | 18 | exon | (NM_018645, |
| chr2 | 239147589 | DMS_GOI | AUC_Δβ_GOI | −1 | | 19 | exon | (NM_018645, |
| chr2 | 239147601 | DMS_GOI | AUC_Δβ_GOI | −1 | | 20 | exon | (NM_018645, |
| chr2 | 239147614 | DMS_GOI | AUC_Δβ_GOI | −1 | | 21 | exon | (NM_018645, |
| chr2 | 239147637 | DMS_GOI | AUC_Δβ_GOI | −1 | | 22 | exon | (NM_018645, |
| chr2 | 239147649 | DMS_GOI | AUC_Δβ_GOI | −1 | | 23 | exon | (NM_018645, |
| chr2 | 239147665 | DMS_GOI | AUC_Δβ_GOI | −1 | | 24 | exon | (NM_018645, |
| chr2 | 239149894 | DMS_GOI | AUC_Δβ_GOI | −1 | | 25 | Intergenic | |
| chr2 | 239149895 | DMS_GOI | AUC_Δβ_GOI | −1 | | 26 | Intergenic | |
| chr3 | 12838468 | DMS_GOI | AUC_Δβ_GOI | −1 | | 27 | intron | (NM_012298, |
| chr3 | 50660608 | DMS_GOI | AUC_Δβ_GOI | 1 | | 28 | intron | (NM_001243925, |
| chr3 | 50670155 | DMS_GOI | AUC_Δβ_GOI | 1 | | 29 | intron | (NM_001243925, |
| chr3 | 128202781 | DMS_GOI | AUC_Δβ_GOI | 1 | | 30 | exon | (NM_032638, |
| chr3 | 128202787 | DMS_GOI | AUC_Δβ_GOI | 1 | | 31 | exon | (NM_032638, |
| chr3 | 128202792 | DMS_GOI | AUC_Δβ_GOI | 1 | | 32 | exon | (NM_032638, |
| chr3 | 128202795 | DMS_GOI | AUC_Δβ_GOI | 1 | | 33 | exon | (NM_032638, |
| chr3 | 128202796 | DMS_GOI | AUC_Δβ_GOI | 1 | | 34 | exon | (NM_032638, |
| chr3 | 128202884 | DMS_GOI | AUC_Δβ_GOI | 1 | | 35 | intron | (NM_001145661, |
| chr3 | 128202894 | DMS_GOI | AUC_Δβ_GOI | 1 | | 36 | intron | (NM_001145661, |
| chr3 | 128202895 | DMS_GOI | AUC_Δβ_GOI | 1 | | 37 | intron | (NM_001145661, |
| chr3 | 128202927 | DMS_GOI | AUC_Δβ_GOI | 1 | | 38 | intron | (NM_001145661, |
| chr3 | 128202928 | DMS_GOI | AUC_Δβ_GOI | 1 | | 39 | intron | (NM_001145661, |
| chr3 | 128205157 | DMS_GOI | AUC_Δβ_GOI | 1 | | 40 | exon | (NM_032638, |
| chr3 | 128205179 | DMS_GOI | AUC_Δβ_GOI | 1 | | 41 | exon | (NM_032638, |
| chr3 | 128205180 | DMS_GOI | AUC_Δβ_GOI | 1 | | 42 | exon | (NM_032638, |
| chr3 | 128205195 | DMS_GOI | AUC_Δβ_GOI | 1 | | 43 | exon | (NM_032638, |
| chr3 | 128205196 | DMS_GOI | AUC_Δβ_GOI | 1 | | 44 | exon | (NM_032638, |
| chr3 | 128205210 | DMS_GOI | AUC_Δβ_GOI | 1 | | 45 | intron | (NM_001145661, |
| chr3 | 128210723 | DMS_GOI | AUC_Δβ_GOI | 1 | | 46 | intron | (NR_125398, |
| chr3 | 128210727 | DMS_GOI | AUC_Δβ_GOI | 1 | | 47 | intron | (NR_125398, |
| chr3 | 128212207 | DMS_GOI | AUC_Δβ_GOI | 1 | | 48 | promoter-TSS | (NM_032638) |
| chr3 | 185817004 | DMS_GOI | AUC_Δβ_GOI | −1 | | 49 | intron | (NM_004454, |
| chr4 | 16084875 | DMS_GOI | AUC_Δβ_GOI | 1 | | 50 | promoter-TSS | (NM_001371407) |
| chr4 | 16084881 | DMS_GOI | AUC_Δβ_GOI | 1 | | 51 | promoter-TSS | (NM_001371407) |
| chr4 | 16084898 | DMS_GOI | AUC_Δβ_GOI | 1 | | 52 | promoter-TSS | (NM_001371407) |
| chr4 | 16085260 | DMS_GOI | AUC_Δβ_GOI | 1 | | 53 | promoter-TSS | (NM_001145852) |
| chr4 | 16085380 | DMS_GOI | AUC_Δβ_GOI | 1 | | 54 | promoter-TSS | (NM_001145852) |
| chr4 | 16085396 | DMS_GOI | AUC_Δβ_GOI | 1 | | 55 | promoter-TSS | (NM_001145852) |
| chr4 | 16085398 | DMS_GOI | AUC_Δβ_GOI | 1 | | 56 | promoter-TSS | (NM_001145852) |
| chr4 | 16085401 | DMS_GOI | AUC_Δβ_GOI | 1 | | 57 | promoter-TSS | (NM_001145852) |
| chr5 | 172661872 | DMS_GOI | AUC_Δβ_GOI | 1 | | 58 | exon | (NM_004387, |
| chr5 | 172661874 | DMS_GOI | AUC_Δβ_GOI | 1 | | 59 | exon | (NM_004387, |
| chr6 | 34522044 | DMS_GOI | AUC_Δβ_GOI | 1 | | 60 | intron | (NM_012391, |
| chr6 | 34523223 | DMS_GOI | AUC_Δβ_GOI | 1 | | 61 | intron | (NM_012391, |
| chr8 | 11561726 | DMS_GOI | AUC_Δβ_GOI | 1 | | 62 | promoter-TSS | (NM_002052) |
| chr8 | 11565855 | DMS_GOI | AUC_Δβ_GOI | 1 | | 63 | exon | (NM_002052, |
| chrS | 11566229 | DMS_GOI | AUC_Δβ_GOI | 1 | | 64 | exon | (NM_002052, |
| chr8 | 11583613 | DMS_GOI | AUC_Δβ_GOI | −1 | | 65 | intron | (NM_002052, |
| chr8 | 11583614 | DMS_GOI | AUC_Δβ_GOI | −1 | | 66 | intron | (NM_002052, |
| chr9 | 126775063 | DMS_GOI | AUC_Δβ_GOI | 1 | | 67 | intron | (NM_004789, |
| chr9 | 126775082 | DMS_GOI | AUC_Δβ_GOI | 1 | | 68 | intron | (NM_004789, |
| chr9 | 126776013 | DMS_GOI | AUC_Δβ_GOI | 1 | | 69 | intron | (NM_004789, |
| chr9 | 126776231 | DMS_GOI | AUC_Δβ_GOI | 1 | | 70 | intron | (NM_004789, |
| chr9 | 139437635 | DMS_GOI | AUC_Δβ_GOI | −1 | | 71 | intron | (NM_017617, |
| chr9 | 139437679 | DMS_GOI | AUC_Δβ_GOI | −1 | | 72 | intron | (NM_017617, |
| chr10 | 23481414 | DMS_GOI | AUC_Δβ_GOI | 1 | | 73 | 5′ | UTR |
| chr10 | 23481415 | DMS_GOI | AUC_Δβ_GOI | 1 | | 74 | 5′ | UTR |
| chr10 | 23481417 | DMS_GOI | AUC_Δβ_GOI | 1 | | 75 | 5′ | UTR |
| chr10 | 23481445 | DMS_GOI | AUC_Δβ_GOI | 1 | | 76 | 5′ | UTR |
| chr10 | 23481447 | DMS_GOI | AUC_Δβ_GOI | 1 | | 77 | 5′ | UTR |
| chr10 | 23481448 | DMS_GOI | AUC_Δβ_GOI | 1 | | 78 | 5′ | UTR |
| chr10 | 23481451 | DMS_GOI | AUC_Δβ_GOI | 1 | | 79 | 5′ | UTR |
| chr10 | 23481461 | DMS_GOI | AUC_Δβ_GOI | 1 | | 80 | exon | (NM_178161, |
| chr10 | 23481462 | DMS_GOI | AUC_Δβ_GOI | 1 | | 81 | exon | (NM_178161, |
| chr10 | 23481463 | DMS_GOI | AUC_Δβ_GOI | 1 | | 82 | exon | (NM_178161, |
| chr10 | 56699066 | DMS_GOI | AUC_Δβ_GOI | −1 | | 83 | intron | (NM_001354404, |
| chr10 | 56699077 | DMS_GOI | AUC_Δβ_GOI | −1 | | 84 | intron | (NM_001354404, |
| chr10 | 57391015 | DMS_GOI | AUC_Δβ_GOI | 1 | | 85 | Intergenic | |
| chr11 | 69451450 | DMS_GOI | AUC_Δβ_GOI | 1 | | 86 | Intergenic | |
| chr12 | 103344993 | DMS_GOI | AUC_Δβ_GOI | −1 | | 87 | intron | (NM_001354304, |
| chr12 | 103344998 | DMS_GOI | AUC_Δβ_GOI | −1 | | 88 | intron | (NM_001354304, |
| chr12 | 103345006 | DMS_GOI | AUC_Δβ_GOI | −1 | | 89 | intron | (NM_001354304, |
| chr12 | 103345029 | DMS_GOI | AUC_Δβ_GOI | −1 | | 90 | intron | (NM_001354304, |
| chr12 | 103345033 | DMS_GOI | AUC_Δβ_GOI | −1 | | 91 | intron | (NM_001354304, |
| chr14 | 51560040 | DMS_GOI | AUC_Δβ_GOI | −1 | | 92 | intron | (NM_052978, |
| chr14 | 51560162 | DMS_GOI | AUC_Δβ_GOI | −1 | | 93 | intron | (NM_052978, |
| chr14 | 51560191 | DMS_GOI | AUC_Δβ_GOI | −1 | | 94 | intron | (NM_052978, |
| chr14 | 51560193 | DMS_GOI | AUC_Δβ_GOI | −1 | | 95 | intron | (NM_052978, |
| chr14 | 51560208 | DMS_GOI | AUC_Δβ_GOI | −1 | | 96 | intron | (NM_052978, |
| chr14 | 51560259 | DMS_GOI | AUC_Δβ_GOI | −1 | | 97 | intron | (NM_052978, |
| chr14 | 51560261 | DMS_GOI | AUC_Δβ_GOI | −1 | | 98 | intron | (NM_052978, |
| chr14 | 51560275 | DMS_GOI | AUC_Δβ_GOI | −1 | | 99 | intron | (NM_052978, |
| chr14 | 65409361 | DMS_GOI | AUC_Δβ_GOI | −1 | | 100 | promoter-TSS | (NM_002083) |
| chr14 | 93385930 | DMS_GOI | AUC_Δβ_GOI | 1 | | 101 | Intergenic | |
| chr14 | 93423499 | DMS_GOI | AUC_Δβ_GOI | −1 | | 102 | intron | (NM_001142593, |
| chr14 | 93423555 | DMS_GOI | AUC_Δβ_GOI | −1 | | 103 | intron | (NM_001142593, |
| chr14 | 93425158 | DMS_GOI | AUC_Δβ_GOI | −1 | | 104 | intron | (NM_001142593, |
| chr14 | 93425189 | DMS_GOI | AUC_Δβ_GOI | −1 | | 105 | intron | (NM_001142593, |
| chr14 | 93425207 | DMS_GOI | AUC_Δβ_GOI | −1 | | 106 | intron | (NM_001142593, |
| chr14 | 93425289 | DMS_GOI | AUC_Δβ_GOI | −1 | | 107 | intron | (NM_001142593, |
| chr14 | 93425328 | DMS_GOI | AUC_Δβ_GOI | −1 | | 108 | intron | (NM_001142593, |
| chr14 | 93425517 | DMS_GOI | AUC_Δβ_GOI | −1 | | 109 | intron | (NM_001142593, |
| chr14 | 93425684 | DMS_GOI | AUC_Δβ_GOI | −1 | | 110 | intron | (NM_001142593, |
| chr14 | 93425685 | DMS_GOI | AUC_Δβ_GOI | −1 | | 111 | intron | (NM_001142593, |
| chr14 | 93425738 | DMS_GOI | AUC_Δβ_GOI | −1 | | 112 | intron | (NM_001142593, |
| chr14 | 93426226 | DMS_GOI | AUC_Δβ_GOI | −1 | | 113 | intron | (NM_001142593, |
| chr14 | 93426244 | DMS_GOI | AUC_Δβ_GOI | −1 | | 114 | intron | (NM_001142593, |
| chr14 | 93426333 | DMS_GOI | AUC_Δβ_GOI | −1 | | 115 | intron | (NM_001142593, |
| chr14 | 93426378 | DMS_GOI | AUC_Δβ_GOI | −1 | | 116 | intron | (NM_001142593, |
| chr14 | 93426872 | DMS_GOI | AUC_Δβ_GOI | −1 | | 117 | intron | (NM_001142593, |
| chr14 | 93426913 | DMS_GOI | AUC_Δβ_GOI | −1 | | 118 | intron | (NM_001142593, |
| chr14 | 93426914 | DMS_GOI | AUC_Δβ_GOI | −1 | | 119 | intron | (NM_001142593, |
| chr14 | 93426959 | DMS_GOI | AUC_Δβ_GOI | −1 | | 120 | intron | (NM_001142593, |
| chr14 | 93439910 | DMS_GOI | AUC_Δβ_GOI | 1 | | 121 | intron | (NM_001142593, |
| chr18 | 31323189 | DMS_GOI | AUC_Δβ_GOI | −1 | | 122 | exon | (NM_030632, |
| chr18 | 31323202 | DMS_GOI | AUC_Δβ_GOI | −1 | | 123 | exon | (NM_030632, |
| chr18 | 53175324 | DMS_GOI | AUC_Δβ_GOI | −1 | | 124 | intron | (NM_001369572, |
| chr18 | 53305364 | DMS_GOI | AUC_Δβ_GOI | −1 | | 125 | Intergenic | |
| chr18 | 53305490 | DMS_GOI | AUC_Δβ_GOI | −1 | | 126 | Intergenic | |
| chr19 | 11086495 | DMS_GOI | AUC_Δβ_GOI | 1 | | 127 | intron | (NM_003072, |
| chr19 | 39993532 | DMS_GOI | AUC_Δβ_GOI | 1 | | 128 | exon | (NM_016941, |
| chr19 | 39993551 | DMS_GOI | AUC_Δβ_GOI | 1 | | 129 | exon | (NM_016941, |
| chr19 | 39993567 | DMS_GOI | AUC_Δβ_GOI | 1 | | 130 | exon | (NM_016941, |
| chr19 | 39993606 | DMS_GOI | AUC_Δβ_GOI | 1 | | 131 | exon | (NM_016941, |
| chr19 | 42618585 | DMS_GOI | AUC_Δβ_GOI | −1 | | 132 | intron | (NM_002698, |
| chr19 | 42618760 | DMS_GOI | AUC_Δβ_GOI | −1 | | 133 | intron | (NM_002698, |
| chr19 | 42618787 | DMS_GOI | AUC_Δβ_GOI | −1 | | 134 | intron | (NM_002698, |
| chr19 | 42618789 | DMS_GOI | AUC_Δβ_GOI | −1 | | 135 | intron | (NM_002698, |
| chr19 | 42618823 | DMS_GOI | AUC_Δβ_GOI | −1 | | 136 | intron | (NM_002698, |
| chr20 | 20232309 | DMS_GOI | AUC_Δβ_GOI | −1 | | 137 | exon | (NM_015585, |
| chr20 | 20232319 | DMS_GOI | AUC_Δβ_GOI | −1 | | 138 | exon | (NM_015585, |
| chr20 | 20232333 | DMS_GOI | AUC_Δβ_GOI | −1 | | 139 | exon | (NM_015585, |
| chr20 | 20232347 | DMS_GOI | AUC_Δβ_GOI | −1 | | 140 | exon | (NM_015585, |
| chr20 | 20232348 | DMS_GOI | AUC_Δβ_GOI | −1 | | 141 | exon | (NM_015585, |
| chr20 | 20232351 | DMS_GOI | AUC_Δβ_GOI | −1 | | 142 | exon | (NM_015585, |
| chr20 | 20232352 | DMS_GOI | AUC_Δβ_GOI | −1 | | 143 | exon | (NM_015585, |
| chr20 | 20232357 | DMS_GOI | AUC_Δβ_GOI | −1 | | 144 | exon | (NM_015585, |
| chr20 | 20232358 | DMS_GOI | AUC_Δβ_GOI | −1 | | 145 | exon | (NM_015585, |
| chr20 | 20232367 | DMS_GOI | AUC_Δβ_GOI | −1 | | 146 | exon | (NM_015585, |
| chr20 | 20232388 | DMS_GOI | AUC_Δβ_GOI | −1 | | 147 | exon | (NM_015585, |
| chr20 | 20266415 | DMS_GOI | AUC_Δβ_GOI | 1 | | 148 | intron | (NM_015585, |
| chr20 | 20376954 | DMS_GOI | AUC_Δβ_GOI | −1 | | 149 | intron | (NM_020343, |
| chr20 | 20376969 | DMS_GOI | AUC_Δβ_GOI | −1 | | 150 | intron | (NM_020343, |
| chr20 | 20377086 | DMS_GOI | AUC_Δβ_GOI | −1 | | 151 | intron | (NM_020343, |
| chr20 | 20377158 | DMS_GOI | AUC_Δβ_GOI | −1 | | 152 | intron | (NM_020343, |
| chr20 | 20377165 | DMS_GOI | AUC_Δβ_GOI | −1 | | 153 | intron | (NM_020343, |
| chr20 | 20421292 | DMS_GOI | AUC_Δβ_GOI | −1 | | 154 | intron | (NM_020343, |
| chr20 | 20421311 | DMS_GOI | AUC_Δβ_GOI | −1 | | 155 | intron | (NM_020343, |
| chr20 | 20421456 | DMS_GOI | AUC_Δβ_GOI | −1 | | 156 | intron | (NM_020343, |
| chr20 | 20421483 | DMS_GOI | AUC_Δβ_GOI | −1 | | 157 | intron | (NM_020343, |
| chr20 | 20433160 | DMS_GOI | AUC_Δβ_GOI | −1 | | 158 | intron | (NM_020343, |
| chr20 | 20433163 | DMS_GOI | AUC_Δβ_GOI | −1 | | 159 | intron | (NM_020343, |
| chr20 | 20433166 | DMS_GOI | AUC_Δβ_GOI | −1 | | 160 | intron | (NM_020343, |
| chr20 | 20433198 | DMS_GOI | AUC_Δβ_GOI | −1 | | 161 | intron | (NM_020343, |
| chr20 | 20433208 | DMS_GOI | AUC_Δβ_GOI | −1 | | 162 | intron | (NM_020343, |
| chr20 | 20433248 | DMS_GOI | AUC_Δβ_GOI | −1 | | 163 | intron | (NM_020343, |
| chr20 | 20433256 | DMS_GOI | AUC_Δβ_GOI | −1 | | 164 | intron | (NM_020343, |
| chr20 | 20433263 | DMS_GOI | AUC_Δβ_GOI | −1 | | 165 | intron | (NM_020343, |
| chr20 | 20433284 | DMS_GOI | AUC_Δβ_GOI | −1 | | 166 | intron | (NM_020343, |
| chr20 | 20433293 | DMS_GOI | AUC_Δβ_GOI | −1 | | 167 | intron | (NM_020343, |
| chr20 | 21490704 | DMS_GOI | AUC_Δβ_GOI | 1 | | 168 | TTS | (NM_002509) |
| chr20 | 21490799 | DMS_GOI | AUC_Δβ_GOI | 1 | | 169 | TTS | (NM_002509) |
| chr20 | 21490807 | DMS_GOI | AUC_Δβ_GOI | 1 | | 170 | TTS | (NM_002509) |
| chr20 | 21494139 | DMS_GOI | AUC_Δβ_GOI | 1 | | 171 | exon | (NM_002509, |
| chr20 | 21494141 | DMS_GOI | AUC_Δβ_GOI | 1 | | 172 | exon | (NM_002509, |
| chr20 | 21494237 | DMS_GOI | AUC_Δβ_GOI | 1 | | 173 | exon | (NM_002509, |
| chr21 | 22564429 | DMS_GOI | AUC_Δβ_GOI | −1 | | 174 | intron | (NM_001352597, |
| chr21 | 42850711 | DMS_GOI | AUC_Δβ_GOI | 1 | | 175 | intron | (NM_005656, |
| chr21 | 42860322 | DMS_GOI | AUC_Δβ_GOI | 1 | | 176 | exon | (NM_001135099, |
| chr21 | 42860341 | DMS_GOI | AUC_Δβ_GOI | 1 | | 177 | exon | (NM_001135099, |
| chr21 | 42880610 | DMS_GOI | AUC_Δβ_GOI | 1 | | 178 | promoter-TSS | (NM_001135099) |
| chr21 | 42880614 | DMS_GOI | AUC_Δβ_GOI | 1 | | 179 | promoter-TSS | (NM_001135099) |
| chr21 | 42880659 | DMS_GOI | AUC_Δβ_GOI | 1 | | 180 | promoter-TSS | (NM_001135099) |
| chr21 | 42880668 | DMS_GOI | AUC_Δβ_GOI | 1 | | 181 | promoter-TSS | (NM_001135099) |
| chr1 | 3052478 | DMS_GOI | AUC_Δβ_TS | −1 | | 182 | intron | (NM_199454, |
| chr1 | 26735081 | DMS_GOI | AUC_Δβ_TS | 1 | | 183 | Intergenic | |
| chr1 | 26735241 | DMS_GOI | AUC_Δβ_TS | 1 | | 184 | Intergenic | |
| chr1 | 26735243 | DMS_GOI | AUC_Δβ_TS | 1 | | 185 | Intergenic | |
| chr1 | 26735251 | DMS_GOI | AUC_Δβ_TS | 1 | | 186 | Intergenic | |
| chr1 | 65721361 | DMS_GOI | AUC_Δβ_TS | −1 | | 187 | Intergenic | |
| chr1 | 229195756 | DMS_GOI | AUC_Δβ_TS | 1 | | 188 | Intergenic | |
| chr2 | 23807028 | DMS_GOI | AUC_Δβ_TS | −1 | | 189 | intron | (NM_052920, |
| chr2 | 171679652 | DMS_GOI | AUC_Δβ_TS | 1 | | 190 | intron | (NM_013445, |
| chr2 | 236877206 | DMS_GOI | AUC_Δβ_TS | −1 | | 191 | exon | (NM_001037131, |
| chr2 | 242549633 | DMS_GOI | AUC_Δβ_TS | 1 | | 192 | intron | (NM_015963, |
| chr2 | 242549635 | DMS_GOI | AUC_Δβ_TS | 1 | | 193 | intron | (NM_015963, |
| chr2 | 242549638 | DMS_GOI | AUC_Δβ_TS | 1 | | 194 | intron | (NM_015963, |
| chr2 | 242549659 | DMS_GOI | AUC_Δβ_TS | 1 | | 195 | intron | (NM_015963, |
| chr3 | 26664523 | DMS_GOI | AUC_Δβ_TS | 1 | | 196 | promoter-TSS | (NM_001317808) |
| chr3 | 126664525 | DMS_GOI | AUC_Δβ_TS | 1 | | 197 | promoter-TSS | (NM_001317808) |
| chr3 | 111431556 | DMS_GOI | AUC_Δβ_TS | −1 | | 198 | intron | (NM_001185106, |
| chr4 | 169799044 | DMS_GOI | AUC_Δβ_TS | 1 | | 199 | exon | (NM_001166110, |
| chr5 | 138441347 | DMS_GOI | AUC_Δβ_TS | −1 | | 200 | intron | (NM_001037633, |
| chr5 | 138441374 | DMS_GOI | AUC_Δβ_TS | −1 | | 201 | intron | (NM_001037633, |
| chr5 | 138441376 | DMS_GOI | AUC_Δβ_TS | −1 | | 202 | intron | (NM_001037633, |
| chr5 | 176038510 | DMS_GOI | AUC_Δβ_TS | 1 | | 203 | Intergenic | |
| chr5 | 176038524 | DMS_GOI | AUC_Δβ_TS | 1 | | 204 | Intergenic | |
| chr5 | 176038525 | DMS_GOI | AUC_Δβ_TS | 1 | | 205 | Intergenic | |
| chr5 | 176038562 | DMS_GOI | AUC_Δβ_TS | 1 | | 206 | Intergenic | |
| chr5 | 179401799 | DMS_GOI | AUC_Δβ_TS | −1 | | 207 | intron | (NM_018434, |
| chr6 | 15265454 | DMS_GOI | AUC_Δβ_TS | 1 | | 208 | intron | (NM_001267040, |
| chr6 | 168462402 | DMS_GOI | AUC_Δβ_TS | −1 | | 209 | intron | (NR_110312, |
| chr6 | 168462447 | DMS_GOI | AUC_Δβ_TS | −1 | | 210 | intron | (NR_110312, |
| chr6 | 170578588 | DMS_GOI | AUC_Δβ_TS | −1 | | 211 | intron | (NR_126021, |
| chr7 | 2968311 | DMS_GOI | AUC_Δβ_TS | −1 | | 212 | exon | (NM_032415, |
| chr7 | 2968441 | DMS_GOI | AUC_Δβ_TS | −1 | | 213 | intron | (NM_001324281, |
| chr7 | 2968442 | DMS_GOI | AUC_Δβ_TS | −1 | | 214 | intron | (NM_001324281, |
| chr7 | 2968451 | DMS_GOI | AUC_Δβ_TS | −1 | | 215 | intron | (NM_001324281, |
| chr7 | 2968454 | DMS_GOI | AUC_Δβ_TS | −1 | | 216 | intron | (NM_001324281, |
| chr7 | 5572006 | DMS_GOI | AUC_Δβ_TS | 1 | | 217 | Intergenic | |
| chr7 | 150812220 | DMS_GOI | AUC_Δβ_TS | 1 | | 218 | exon | (NM_001308305, |
| chr8 | 47570600 | DMS_GOI | AUC_Δβ_TS | −1 | | 219 | Intergenic | |
| chr9 | 116411412 | DMS_GOI | AUC_Δβ_TS | −1 | | 220 | Intergenic | |
| chr9 | 124498726 | DMS_GOI | AUC_Δβ_TS | 1 | | 221 | intron | (NM_032552, |
| chr9 | 124620057 | DMS_GOI | AUC_Δβ_TS | −1 | | 222 | intron | (NM_001139442, |
| chr10 | 676777 | DMS_GOI | AUC_Δβ_TS | −1 | | 223 | non-coding | (NR_147612, |
| chr10 | 676811 | DMS_GOI | AUC_Δβ_TS | −1 | | 224 | non-coding | (NR_147612, |
| chr11 | 2000638 | DMS_GOI | AUC_Δβ_TS | −1 | | 225 | Intergenic | |
| chr11 | 11600192 | DMS_GOI | AUC_Δβ_TS | −1 | | 226 | intron | (NM_001363464, |
| chr11 | 11600210 | DMS_GOI | AUC_Δβ_TS | −1 | | 227 | intron | (NM_001363464, |
| chr11 | 11600228 | DMS_GOI | AUC_Δβ_TS | −1 | | 228 | intron | (NM_001363464, |
| chr11 | 11600230 | DMS_GOI | AUC_Δβ_TS | −1 | | 229 | intron | (NM_001363464, |
| chr11 | 11600231 | DMS_GOI | AUC_Δβ_TS | −1 | | 230 | intron | (NM_001363464, |
| chr11 | 11600240 | DMS_GOI | AUC_Δβ_TS | −1 | | 231 | intron | (NM_001363464, |
| chr11 | 64120601 | DMS_GOI | AUC_Δβ_TS | −1 | | 232 | exon | (NM_032251, |
| chr11 | 70851727 | DMS_GOI | AUC_Δβ_TS | −1 | | 233 | intron | (NM_012309, |
| chr12 | 40618451 | DMS_GOI | AUC_Δβ_TS | 1 | | 234 | promoter-TSS | (NM_198578) |
| chr12 | 40618493 | DMS_GOI | AUC_Δβ_TS | 1 | | 235 | promoter-TSS | (NM_198578) |
| chr15 | 32385689 | DMS_GOI | AUC_Δβ_TS | −1 | | 236 | intron | (NM_000746, |
| chr15 | 68723747 | DMS_GOI | AUC_Δβ_TS | 1 | | 237 | intron | (NM_001004439, |
| chr15 | 89954601 | DMS_GOI | AUC_Δβ_TS | 1 | | 238 | intron | (NR_160724, |
| chr15 | 89954603 | DMS_GOI | AUC_Δβ_TS | 1 | | 239 | intron | (NR_160724, |
| chr15 | 89954605 | DMS_GOI | AUC_Δβ_TS | 1 | | 240 | intron | (NR_160724, |
| chr15 | 89959942 | DMS_GOI | AUC_Δβ_TS | 1 | | 241 | intron | (NR_160724, |
| chr16 | 85498200 | DMS_GOI | AUC_Δβ_TS | −1 | | 242 | Intergenic | |
| chr16 | 85498219 | DMS_GOI | AUC_Δβ_TS | −1 | | 243 | Intergenic | |
| chr16 | 85498220 | DMS_GOI | AUC_Δβ_TS | −1 | | 244 | Intergenic | |
| chr16 | 85498227 | DMS_GOI | AUC_Δβ_TS | −1 | | 245 | Intergenic : | |
| chr16 | 85498781 | DMS_GOI | AUC_Δβ_TS | −1 | | 246 | Intergenic | |
| chr16 | 85519155 | DMS_GOI | AUC_Δβ_TS | −1 | | 247 | Intergenic | |
| chr16 | 85519162 | DMS_GOI | AUC_Δβ_TS | −1 | | 248 | Intergenic | |
| chr16 | 85519165 | DMS_GOI | AUC_Δβ_TS | −1 | | 249 | Intergenic | |
| chr16 | 85519168 | DMS_GOI | AUC_Δβ_TS | −1 | | 250 | Intergenic | |
| chr16 | 85519171 | DMS_GOI | AUC_Δβ_TS | −1 | | 251 | Intergenic | |
| chr16 | 85519173 | DMS_GOI | AUC_Δβ_TS | −1 | | 252 | Intergenic | |
| chr16 | 85519303 | DMS_GOI | AUC_Δβ_TS | −1 | | 253 | Intergenic | |
| chr16 | 88715953 | DMS_GOI | AUC_Δβ_TS | −1 | | 254 | intron | (NM_000101, |
| chr17 | 28299568 | DMS_GOI | AUC_Δβ_TS | −1 | | 255 | intron | (NR_026738, |
| chr17 | 80372745 | DMS_GOI | AUC_Δβ_TS | 1 | | 256 | intron | (NR_033265, |
| chr17 | 80372760 | DMS_GOI | AUC_Δβ_TS | 1 | | 257 | intron | (NR_033265, |
| chr17 | 80372761 | DMS_GOI | AUC_Δβ_TS | 1 | | 258 | intron | (NR_033265, |
| chr17 | 80372766 | DMS_GOI | AUC_Δβ_TS | 1 | | 259 | intron | (NR_033265, |
| chr17 | 80372767 | DMS_GOI | AUC_Δβ_TS | 1 | | 260 | intron | (NR_033265, |
| chr17 | 80372770 | DMS_GOI | AUC_Δβ_TS | 1 | | 261 | intron | (NR_033265, |
| chr17 | 80372771 | DMS_GOI | AUC_Δβ_TS | 1 | | 262 | intron | (NR_033265, |
| chr17 | 80372793 | DMS_GOI | AUC_Δβ_TS | 1 | | 263 | intron | (NR_033265, |
| chr18 | 57364080 | DMS_GOI | AUC_Δβ_TS | 1 | | 264 | intron | (NM_133459, |
| chr19 | 591647 | DMS_GOI | AUC_Δβ_TS | 1 | | 265 | intron | (NM_001194, |
| chr19 | 591656 | DMS_GOI | AUC_Δβ_TS | 1 | | 266 | intron | (NM_001194, |
| chr19 | 591660 | DMS_GOI | AUC_Δβ_TS | 1 | | 267 | intron | (NM_001194, |
| chr19 | 591662 | DMS_GOI | AUC_Δβ_TS | 1 | | 268 | intron | (NM_001194, |
| chr19 | 591682 | DMS_GOI | AUC_Δβ_TS | 1 | | 269 | intron | (NM_001194, |
| chr19 | 591683 | DMS_GOI | AUC_Δβ_TS | 1 | | 270 | intron | (NM_001194, |
| chr19 | 591684 | DMS_GOI | AUC_Δβ_TS | 1 | | 271 | intron | (NM_001194, |
| chr19 | 591685 | DMS_GOI | AUC_Δβ_TS | 1 | | 272 | intron | (NM_001194, |
| chr19 | 591688 | DMS_GOI | AUC_Δβ_TS | 1 | | 273 | intron | (NM_001194, |
| chr19 | 18837089 | DMS_GOI | AUC_Δβ_TS | −1 | | 274 | intron | (NM_001098482, |
| chr19 | 18837102 | DMS_GOI | AUC_Δβ_TS | −1 | | 275 | intron | (NM_001098482, |
| chr20 | 3778162 | DMS_GOI | AUC_Δβ_TS | −1 | | 276 | intron | (NM_004358, |
| chr20 | 32156432 | DMS_GOI | AUC_Δβ_TS | −1 | | 277 | intron | (NM_001039709, |
| chr20 | 48099327 | DMS_GOI | AUC_Δβ_TS | 1 | | 278 | promoter-TSS | (NM_004975) |
| chr20 | 48099339 | DMS_GOI | AUC_Δβ_TS | 1 | | 279 | promoter-TSS | (NM_004975) |
| chrX | 36981727 | DMS_GOI | AUC_Δβ_TS | 1 | | 280 | Intergenic | |
| chrX | 36981729 | DMS_GOI | AUC_Δβ_TS | 1 | | 281 | Intergenic | |
| chr1 | 24392969 | P_DEgenes | ExpMeth | | | 1 | intron | (NM_152372, |
| chr1 | 24410522 | P_DEgenes | ExpMeth | | | 2 | intron | (NM_152372, |
| chr1 | 24434829 | P_DEgenes | ExpMeth | | | 3 | intron | (NM_152372, |
| chr1 | 24438565 | P_DEgenes | ExpMeth | | | 4 | promoter-TSS | (NM_152372) |
| chr1 | 27240357 | P_DEgenes | ExpMeth | | | 5 | promoter-TSS | (NM_021969) |
| chr1 | 50513586 | P_DEgenes | ExpMeth | | | 6 | promoter-TSS | (NM_001324208) |
| chr1 | 50569482 | P_DEgenes | ExpMeth | | | 7 | promoter-TSS | (NM_001144776) |
| chr1 | 50571262 | P_DEgenes | ExpMeth | | | 8 | promoter-TSS | (NM_001144775) |
| chr1 | 50571864 | P_DEgenes | ExpMeth | | | 9 | promoter-TSS | (NM_001144775) |
| chr1 | 50574505 | P_DEgenes | ExpMeth | | | 10 | promoter-TSS | (NM_001144774) |
| chr1 | 50574538 | P_DEgenes | ExpMeth | | | 11 | promoter-TSS | (NM_001144774) |
| chr1 | 50575198 | P_DEgenes | ExpMeth | | | 12 | promoter-TSS | (NM_001324217) |
| chr1 | 50575278 | P_DEgenes | ExpMeth | | | 13 | promoter-TSS | (NM_001324217) |
| chr1 | 50575388 | P_DEgenes | ExpMeth | | | 14 | promoter-TSS | (NM_001324217) |
| chr1 | 50610525 | P_DEgenes | ExpMeth | | | 15 | intron | (NR_136725, |
| chr1 | 50642702 | P_DEgenes | ExpMeth | | | 16 | exon | (NM_001144774, |
| chr1 | 78445126 | P_DEgenes | ExpMeth | | | 17 | promoter-TSS | (NM_001317099) |
| chr1 | 78511486 | P_DEgenes | ExpMeth | | | 18 | promoter-TSS | (NM_017655) |
| chr1 | 114521963 | P_DEgenes | ExpMeth | | | 19 | promoter-TSS | (NM_001286353) |
| chr1 | 114521965 | P_DEgenes | ExpMeth | | | 20 | promoter-TSS | (NM_001286353) |
| chr1 | 114522013 | P_DEgenes | ExpMeth | | | 21 | promoter-TSS | (NM_001286353) |
| chr1 | 114522595 | P_DEgenes | ExpMeth | | | 22 | exon | (NM_001286352, |
| chr1 | 152956457 | P_DEgenes | ExpMeth | | | 23 | promoter-TSS | (NM_001199828) |
| chr1 | 152957607 | P_DEgenes | ExpMeth | | | 24 | exon | (NM_001199828, |
| chr1 | 153330230 | P_DEgenes | ExpMeth | | | 25 | promoter-TSS | (NM_002965) |
| chr2 | 74119341 | P_DEgenes | ExpMeth | | | 26 | promoter-TSS | (NM_001199893) |
| chr2 | 74119978 | P_DEgenes | ExpMeth | | | 27 | promoter-TSS | (NM_001199893) |
| chr2 | 74119994 | P_DEgenes | ExpMeth | | | 28 | promoter-TSS | (NM_001199893) |
| chr2 | 74120006 | P_DEgenes | ExpMeth | | | 29 | promoter-TSS | (NM_001199893) |
| chr2 | 74120009 | P_DEgenes | ExpMeth | | | 30 | promoter-TSS | (NM_001199893) |
| chr2 | 74120035 | P_DEgenes | ExpMeth | | | 31 | promoter-TSS | (NM_001199893) |
| chr2 | 95940101 | P_DEgenes | ExpMeth | | | 32 | promoter-TSS | (NM_001165978) |
| chr2 | 95940121 | P_DEgenes | ExpMeth | | | 33 | promoter-TSS | (NM_001165978) |
| chr2 | 95940140 | P_DEgenes | ExpMeth | | | 34 | promoter-TSS | (NM_001165978) |
| chr2 | 95940145 | P_DEgenes | ExpMeth | | | 35 | promoter-TSS | (NM_001165978) |
| chr2 | 95940149 | P_DEgenes | ExpMeth | | | 36 | promoter-TSS | (NM_001165978) |
| chr2 | 95940627 | P_DEgenes | ExpMeth | | | 37 | intron | (NM_001165977, |
| chr2 | 95943464 | P_DEgenes | ExpMeth | | | 38 | intron | (NM_001165977, |
| chr2 | 95945514 | P_DEgenes | ExpMeth | | | 39 | exon | (NM_001165978, |
| chr2 | 95954155 | P_DEgenes | ExpMeth | | | 40 | exon | (NM_001165978, |
| chr2 | 95954163 | P_DEgenes | ExpMeth | | | 41 | exon | (NM_001165978, |
| chr2 | 169921199 | P_DEgenes | ExpMeth | | | 42 | Intergenic | |
| chr2 | 169923445 | P_DEgenes | ExpMeth | | | 43 | promoter-TSS | (NM_001289763) |
| chr2 | 169923487 | P_DEgenes | ExpMeth | | | 44 | promoter-TSS | (NM_001289763) |
| chr2 | 169925966 | P_DEgenes | ExpMeth | | | 45 | 5′ | UTR |
| chr2 | 169925972 | P_DEgenes | ExpMeth | | | 46 | 5′ | UTR |
| chr2 | 169925986 | P_DEgenes | ExpMeth | | | 47 | 5′ | UTR |
| chr2 | 169928910 | P_DEgenes | ExpMeth | | | 48 | promoter-TSS | (NM_001142270) |
| chr2 | 169937517 | P_DEgenes | ExpMeth | | | 49 | promoter-TSS | (NM_001142271) |
| chr2 | 177001240 | P_DEgenes | ExpMeth | | | 50 | promoter-TSS | (NR_148241) |
| chr2 | 177001585 | P_DEgenes | ExpMeth | | | 51 | non-coding | (NR_038435, |
| chr2 | 177015022 | P_DEgenes | ExpMeth | | | 52 | promoter-TSS | (NR_029609) |
| chr2 | 177025519 | P_DEgenes | ExpMeth | | | 53 | intron | (NM_006898, |
| chr2 | 177028705 | P_DEgenes | ExpMeth | | | 54 | 5′ | UTR |
| chr2 | 182545174 | P_DEgenes | ExpMeth | | | 55 | promoter-TSS | (NR_146176) |
| chr2 | 182545503 | P_DEgenes | ExpMeth | | | 56 | promoter-TSS | (NR_146176) |
| chr2 | 219696414 | P_DEgenes | ExpMeth | | | 57 | promoter-TSS | (NM_017431) |
| chr2 | 219696424 | P_DEgenes | ExpMeth | | | 58 | promoter-TSS | (NM_017431) |
| chr2 | 219696426 | P_DEgenes | ExpMeth | | | 59 | promoter-TSS | (NM_017431) |
| chr2 | 219696439 | P_DEgenes | ExpMeth | | | 60 | promoter-TSS | (NM_017431) |
| chr2 | 219696709 | P_DEgenes | ExpMeth | | | 61 | promoter-TSS | (NM_017431) |
| chr2 | 223916432 | P_DEgenes | ExpMeth | | | 62 | promoter-TSS | (NM_080671) |
| chr2 | 223916762 | P_DEgenes | ExpMeth | | | 63 | 5′ | UTR |
| chr2 | 223916818 | P_DEgenes | ExpMeth | | | 64 | exon | (NM_080671, |
| chr2 | 233924577 | P_DEgenes | ExpMeth | | | 65 | promoter-TSS | (NM_005541) |
| chr2 | 233924962 | P_DEgenes | ExpMeth | | | 66 | promoter-TSS | (NM_005541) |
| chr2 | 233925089 | P_DEgenes | ExpMeth | | | 67 | exon | (NM_005541, |
| chr2 | 233940872 | P_DEgenes | ExpMeth | | | 68 | intron | (NM_001017915, |
| chr2 | 233985212 | P_DEgenes | ExpMeth | | | 69 | intron | (NM_001017915, |
| chr2 | 233986826 | P_DEgenes | ExpMeth | | | 70 | exon | (NM_005541, |
| chr2 | 234053819 | P_DEgenes | ExpMeth | | | 71 | intron | (NM_001017915, |
| chr2 | 234054695 | P_DEgenes | ExpMeth | | | 72 | intron | (NM_001017915, |
| chr2 | 234070270 | P_DEgenes | ExpMeth | | | 73 | exon | (NM_005541, |
| chr2 | 234070335 | P_DEgenes | ExpMeth | | | 74 | exon | (NM_005541, |
| chr2 | 234072847 | P_DEgenes | ExpMeth | | | 75 | intron | (NM_001017915, |
| chr2 | 234078106 | P_DEgenes | ExpMeth | | | 76 | intron | (NM_001017915, |
| chr2 | 234078567 | P_DEgenes | ExpMeth | | | 77 | intron | (NM_001017915, |
| chr2 | 234078920 | P_DEgenes | ExpMeth | | | 78 | intron | (NM_001017915, |
| chr2 | 234103983 | P_DEgenes | ExpMeth | | | 79 | exon | (NM_005541, |
| chr2 | 234113188 | P_DEgenes | ExpMeth | | | 80 | exon | (NM_005541, |
| chr3 | 12837871 | P_DEgenes | ExpMeth | | | 81 | promoter-TSS | (NM_001162499) |
| chr3 | 12838071 | P_DEgenes | ExpMeth | | | 82 | promoter-TSS | (NM_001162499) |
| chr3 | 12838079 | P_DEgenes | ExpMeth | | | 83 | promoter-TSS | (NM_001162499) |
| chr3 | 12848532 | P_DEgenes | ExpMeth | | | 84 | intron | (NM_012298, |
| chr3 | 12868845 | P_DEgenes | ExpMeth | | | 85 | exon | (NM_001162499, |
| chr3 | 42726911 | P_DEgenes | ExpMeth | | | 86 | promoter-TSS | (NM_152393) |
| chr3 | 111717411 | P_DEgenes | ExpMeth | | | 87 | promoter-TSS | (NM_013259) |
| chr3 | 111717486 | P_DEgenes | ExpMeth | | | 88 | promoter-TSS | (NM_013259) |
| chr3 | 111717542 | P_DEgenes | ExpMeth | | | 89 | promoter-TSS | (NM_013259) |
| chr3 | 111717936 | P_DEgenes | ExpMeth | | | 90 | promoter-TSS | (NM_001008273) |
| chr3 | 111718095 | P_DEgenes | ExpMeth | | | 91 | 5′ | UTR |
| chr3 | 111719270 | P_DEgenes | ExpMeth | | | 92 | intron | (NM_001008273, |
| chr3 | 111719519 | P_DEgenes | ExpMeth | | | 93 | exon | (NM_013259, |
| chr3 | 128200821 | P_DEgenes | ExpMeth | | | 94 | intron | (NM_001145661, |
| chr3 | 128206659 | P_DEgenes | ExpMeth | | | 95 | promoter-TSS | (NM_001145662) |
| chr3 | 128207273 | P_DEgenes | ExpMeth | | | 96 | promoter-TSS | (NM_001145662) |
| chr3 | 128209391 | P_DEgenes | ExpMeth | | | 97 | intron | (NR_125398, |
| chr3 | 128211916 | P_DEgenes | ExpMeth | | | 98 | promoter-TSS | (NM_032638) |
| chr3 | 128211928 | P_DEgenes | ExpMeth | | | 99 | promoter-TSS | (NM_032638) |
| chr3 | 150481064 | P_DEgenes | ExpMeth | | | 100 | promoter-TSS | (NM_005067) |
| chr3 | 150481118 | P_DEgenes | ExpMeth | | | 101 | promoter-TSS | (NM_005067) |
| chr3 | 150481164 | P_DEgenes | ExpMeth | | | 102 | promoter-TSS | (NM_005067) |
| chr3 | 151154610 | P_DEgenes | ExpMeth | | | 103 | TTS | (NM_053002) |
| chr3 | 151158459 | P_DEgenes | ExpMeth | | | 104 | promoter-TSS | (NM_001178145) |
| chr3 | 151158468 | P_DEgenes | ExpMeth | | | 105 | promoter-TSS | (NM_001178145) |
| chr3 | 151160838 | P_DEgenes | ExpMeth | | | 106 | exon | (NM_178822, |
| chr3 | 151176397 | P_DEgenes | ExpMeth | | | 107 | promoter-TSS | (NM_178822) |
| chr3 | 181429614 | P_DEgenes | ExpMeth | | | 108 | promoter-TSS | (NM_003106) |
| chr3 | 181429622 | P_DEgenes | ExpMeth | | | 109 | promoter-TSS | (NM_003106) |
| chr3 | 185782249 | P_DEgenes | ExpMeth | | | 110 | intron | (NM_004454, |
| chr3 | 185783659 | P_DEgenes | ExpMeth | | | 111 | exon | (NM_004454, |
| chr3 | 185796794 | P_DEgenes | ExpMeth | | | 112 | intron | (NM_004454, |
| chr3 | 185797595 | P_DEgenes | ExpMeth | | | 113 | exon | (NM_004454, |
| chr3 | 185823179 | P_DEgenes | ExpMeth | | | 114 | exon | (NM_004454, |
| chr3 | 185823413 | P_DEgenes | ExpMeth | | | 115 | intron | (NM_004454, |
| chr3 | 185823631 | P_DEgenes | ExpMeth | | | 116 | 5′ | UTR |
| chr3 | 185826233 | P_DEgenes | ExpMeth | | | 117 | intron | (NM_004454, |
| chr3 | 185826239 | P_DEgenes | ExpMeth | | | 118 | intron | (NM_004454, |
| chr3 | 185826364 | P_DEgenes | ExpMeth | | | 119 | intron | (NM_004454, |
| chr3 | 185826618 | P_DEgenes | ExpMeth | | | 120 | intron | (NM_004454, |
| chr3 | 185826688 | P_DEgenes | ExpMeth | | | 121 | promoter-TSS | (NM_004454) |
| chr3 | 185826770 | P_DEgenes | ExpMeth | | | 122 | promoter-TSS | (NM_004454) |
| chr3 | 185826801 | P_DEgenes | ExpMeth | | | 123 | promoter-TSS | (NM_004454) |
| chr3 | 185828007 | P_DEgenes | ExpMeth | | | 124 | Intergenic | |
| chr3 | 187086020 | P_DEgenes | ExpMeth | | | 125 | promoter-TSS | (NM_022147) |
| chr4 | 6694696 | P_DEgenes | ExpMeth | | | 126 | promoter-TSS | (NM_005980) |
| chr4 | 6695505 | P_DEgenes | ExpMeth | | | 127 | promoter-TSS | (NM_005980) |
| chr4 | 38796154 | P_DEgenes | ExpMeth | | | 128 | Intergenic | |
| chr4 | 38805897 | P_DEgenes | ExpMeth | | | 129 | 5′ | UTR |
| chr4 | 38806310 | P_DEgenes | ExpMeth | | | 130 | promoter-TSS | (NM_003263) |
| chr4 | 38806312 | P_DEgenes | ExpMeth | | | 131 | promoter-TSS | (NM_003263) |
| chr4 | 38806340 | P_DEgenes | ExpMeth | | | 132 | promoter-TSS | (NM_003263) |
| chr4 | 38806372 | P_DEgenes | ExpMeth | | | 133 | promoter-TSS | (NM_003263) |
| chr4 | 38806437 | P_DEgenes | ExpMeth | | | 134 | promoter-TSS | (NM_003263) |
| chr4 | 38807054 | P_DEgenes | ExpMeth | | | 135 | promoter-TSS | (NM_003263) |
| chr4 | 38807099 | P_DEgenes | ExpMeth | | | 136 | promoter-TSS | (NM_003263) |
| chr4 | 38858338 | P_DEgenes | ExpMeth | | | 137 | promoter-TSS | (NM_006068) |
| chr4 | 41258330 | P_DEgenes | ExpMeth | | | 138 | promoter-TSS | (NM_004181) |
| chr4 | 41258686 | P_DEgenes | ExpMeth | | | 139 | promoter-TSS | (NM_004181) |
| chr4 | 41258750 | P_DEgenes | ExpMeth | | | 140 | promoter-TSS | (NM_004181) |
| chr4 | 41258825 | P_DEgenes | ExpMeth | | | 141 | promoter-TSS | (NM_004181) |
| chr4 | 41258828 | P_DEgenes | ExpMeth | | | 142 | promoter-TSS | (NM_004181) |
| chr4 | 41258849 | P_DEgenes | ExpMeth | | | 143 | promoter-TSS | (NM_004181) |
| chr4 | 41258850 | P_DEgenes | ExpMeth | | | 144 | promoter-TSS | (NM_004181) |
| chr4 | 41258857 | P_DEgenes | ExpMeth | | | 145 | promoter-TSS | (NM_004181) |
| chr4 | 41258862 | P_DEgenes | ExpMeth | | | 146 | promoter-TSS | (NM_004181) |
| chr4 | 41258863 | P_DEgenes | ExpMeth | | | 147 | promoter-TSS | (NM_004181) |
| chr4 | 41262556 | P_DEgenes | ExpMeth | | | 148 | intron | (NM_004181, |
| chr4 | 41263656 | P_DEgenes | ExpMeth | | | 149 | exon | (NM_004181, |
| chr4 | 48899120 | P_DEgenes | ExpMeth | | | 150 | intron | (NR_104589, |
| chr4 | 48899765 | P_DEgenes | ExpMeth | | | 151 | exon | (NM_001014446, |
| chr4 | 48908644 | P_DEgenes | ExpMeth | | | 152 | promoter-TSS | (NM_001014446) |
| chr4 | 48908674 | P_DEgenes | ExpMeth | | | 153 | promoter-TSS | (NM_001014446) |
| chr4 | 48908691 | P_DEgenes | ExpMeth | | | 154 | promoter-TSS | (NM_001014446) |
| chr4 | 48908705 | P_DEgenes | ExpMeth | | | 155 | promoter-TSS | (NM_001014446) |
| chr4 | 48908715 | P_DEgenes | ExpMeth | | | 156 | promoter-TSS | (NM_001014446) |
| chr4 | 48908745 | P_DEgenes | ExpMeth | | | 157 | promoter-TSS | (NM_001014446) |
| chr4 | 48908854 | P_DEgenes | ExpMeth | | | 158 | promoter-TSS | (NM_001014446) |
| chr4 | 84030896 | P_DEgenes | ExpMeth | | | 159 | promoter-TSS | (NM_001130716) |
| chr4 | 84035768 | P_DEgenes | ExpMeth | | | 160 | promoter-TSS | (NM_016619) |
| chr4 | 84035789 | P_DEgenes | ExpMeth | | | 161 | promoter-TSS | (NM_016619) |
| chr4 | 84035855 | P_DEgenes | ExpMeth | | | 162 | promoter-TSS | (NM_016619) |
| chr4 | 84058128 | P_DEgenes | ExpMeth | | | 163 | Intergenic | |
| chr4 | 177158625 | P_DEgenes | ExpMeth | | | 164 | intron | (NM_080874, |
| chr4 | 177162727 | P_DEgenes | ExpMeth | | | 165 | intron | (NM_080874, |
| chr4 | 177170609 | P_DEgenes | ExpMeth | | | 166 | intron | (NM_080874, |
| chr4 | 177190273 | P_DEgenes | ExpMeth | | | 167 | promoter-TSS | (NM_080874) |
| chr4 | 177198622 | P_DEgenes | ExpMeth | | | 168 | Intergenic | |
| chr5 | 102089585 | P_DEgenes | ExpMeth | | | 169 | promoter-TSS | (NM_001319943) |
| chr5 | 102090271 | P_DEgenes | ExpMeth | | | 170 | promoter-TSS | (NM_001364586) |
| chr5 | 102090387 | P_DEgenes | ExpMeth | | | 171 | promoter-TSS | (NM_001364586) |
| chr5 | 102090918 | P_DEgenes | ExpMeth | | | 172 | promoter-TSS | (NM_001364586) |
| chr5 | 102090947 | P_DEgenes | ExpMeth | | | 173 | promoter-TSS | (NM_001364586) |
| chr5 | 102143661 | P_DEgenes | ExpMeth | | | 174 | promoter-TSS | (NM_001364582) |
| chr5 | 102143667 | P_DEgenes | ExpMeth | | | 175 | promoter-TSS | (NM_001364582) |
| chr5 | 102200141 | P_DEgenes | ExpMeth | | | 176 | intron | (NM_001364582, |
| chr5 | 102200848 | P_DEgenes | ExpMeth | | | 177 | promoter-TSS | (NM_138822) |
| chr5 | 102200914 | P_DEgenes | ExpMeth | | | 178 | promoter-TSS | (NM_138822) |
| chr5 | 102201083 | P_DEgenes | ExpMeth | | | 179 | promoter-TSS | (NM_138822) |
| chr5 | 102201330 | P_DEgenes | ExpMeth | | | 180 | promoter-TSS | (NM_138822) |
| chr5 | 102201341 | P_DEgenes | ExpMeth | | | 181 | promoter-TSS | (NM_138822) |
| chr5 | 102201423 | P_DEgenes | ExpMeth | | | 182 | promoter-TSS | (NM_138822) |
| chr5 | 102201427 | P_DEgenes | ExpMeth | | | 183 | promoter-TSS | (NM_138822) |
| chr5 | 102201614 | P_DEgenes | ExpMeth | | | 184 | non-coding | (NR_033440, |
| chr5 | 102201661 | P_DEgenes | ExpMeth | | | 185 | non-coding | (NR_033440, |
| chr5 | 102201720 | P_DEgenes | ExpMeth | | | 186 | non-coding | (NR_033440, |
| chr5 | 102202191 | P_DEgenes | ExpMeth | | | 187 | intron | (NM_001364584, |
| chr5 | 102231517 | P_DEgenes | ExpMeth | | | 188 | intron | (NR_033440, |
| chr5 | 102285181 | P_DEgenes | ExpMeth | | | 189 | exon | (NM_001364582, |
| chr5 | 102295601 | P_DEgenes | ExpMeth | | | 190 | exon | (NM_001364582, |
| chr5 | 102325375 | P_DEgenes | ExpMeth | | | 191 | intron | (NM_001364591, |
| chr5 | 102343057 | P_DEgenes | ExpMeth | | | 192 | intron | (NM_001364584, |
| chr5 | 102343146 | P_DEgenes | ExpMeth | | | 193 | exon | (NM_001364582, |
| chr5 | 102343163 | P_DEgenes | ExpMeth | | | 194 | exon | (NM_001364582, |
| chr5 | 102343173 | P_DEgenes | ExpMeth | | | 195 | exon | (NM_001364582, |
| chr6 | 34523991 | P_DEgenes | ExpMeth | | | 196 | promoter-TSS | (NM_001252294) |
| chr6 | 34524010 | P_DEgenes | ExpMeth | | | 197 | promoter-TSS | (NM_001252294) |
| chr6 | 112451090 | P_DEgenes | ExpMeth | | | 198 | exon | (NM_002290, |
| chr6 | 112451261 | P_DEgenes | ExpMeth | | | 199 | intron | (NM_001105206, |
| chr6 | 112460378 | P_DEgenes | ExpMeth | | | 200 | exon | (NM_002290, |
| chr6 | 112486266 | P_DEgenes | ExpMeth | | | 201 | exon | (NM_002290, |
| chr6 | 112500604 | P_DEgenes | ExpMeth | | | 202 | intron | (NM_001105206, |
| chr6 | 112512866 | P_DEgenes | ExpMeth | | | 203 | exon | (NM_002290, |
| chr6 | 112512903 | P_DEgenes | ExpMeth | | | 204 | exon | (NM_002290, |
| chr6 | 112512913 | P_DEgenes | ExpMeth | | | 205 | exon | (NM_002290, |
| chr6 | 112537550 | P_DEgenes | ExpMeth | | | 206 | exon | (NM_002290, |
| chr6 | 112537855 | P_DEgenes | ExpMeth | | | 207 | intron | (NM_001105206, |
| chr6 | 112575587 | P_DEgenes | ExpMeth | | | 208 | promoter-TSS | (NM_002290) |
| chr6 | 112575640 | P_DEgenes | ExpMeth | | | 209 | promoter-TSS | (NM_002290) |
| chr6 | 112575674 | P_DEgenes | ExpMeth | | | 210 | promoter-TSS | (NM_002290) |
| chr6 | 112575680 | P_DEgenes | ExpMeth | | | 211 | promoter-TSS | (NM_002290) |
| chr6 | 112575690 | P_DEgenes | ExpMeth | | | 212 | promoter-TSS | (NM_002290) |
| chr6 | 112575702 | P_DEgenes | ExpMeth | | | 213 | promoter-TSS | (NM_002290) |
| chr6 | 112575749 | P_DEgenes | ExpMeth | | | 214 | promoter-TSS | (NM_002290) |
| chr6 | 112575812 | P_DEgenes | ExpMeth | | | 215 | promoter-TSS | (NM_002290) |
| chr6 | 112575817 | P_DEgenes | ExpMeth | | | 216 | promoter-TSS | (NM_002290) |
| chr6 | 112575824 | P_DEgenes | ExpMeth | | | 217 | promoter-TSS | (NM_002290) |
| chr6 | 112576041 | P_DEgenes | ExpMeth | | | 218 | promoter-TSS | (NM_002290) |
| chr7 | 148508815 | P_DEgenes | ExpMeth | | | 219 | intron | (NM_004456, |
| chr7 | 148580501 | P_DEgenes | ExpMeth | | | 220 | promoter-TSS | (NM_001203249) |
| chr7 | 148581151 | P_DEgenes | ExpMeth | | | 221 | promoter-TSS | (NM_001203249) |
| chr7 | 148581237 | P_DEgenes | ExpMeth | | | 222 | promoter-TSS | (NM_001203247) |
| chr7 | 148581240 | P_DEgenes | ExpMeth | | | 223 | promoter-TSS | (NM_001203247) |
| chr7 | 148581270 | P_DEgenes | ExpMeth | | | 224 | promoter-TSS | (NM_001203247) |
| chr7 | 148581275 | P_DEgenes | ExpMeth | | | 225 | promoter-TSS | (NM_001203247) |
| chr7 | 148581279 | P_DEgenes | ExpMeth | | | 226 | promoter-TSS | (NM_001203247) |
| chr7 | 148581313 | P_DEgenes | ExpMeth | | | 227 | promoter-TSS | (NM_001203247) |
| chr8 | 17169082 | P_DEgenes | ExpMeth | | | 228 | intron | (NM_004686, |
| chr8 | 17169272 | P_DEgenes | ExpMeth | | | 229 | intron | (NM_004686, |
| chr8 | 17206466 | P_DEgenes | ExpMeth | | | 230 | exon | (NM_004686, |
| chr8 | 17206646 | P_DEgenes | ExpMeth | | | 231 | intron | (NM_004686, |
| chr8 | 17206894 | P_DEgenes | ExpMeth | | | 232 | intron | (NM_004686, |
| chr8 | 17270716 | P_DEgenes | ExpMeth | | | 233 | promoter-TSS | (NM_004686) |
| chr8 | 17270736 | P_DEgenes | ExpMeth | | | 234 | promoter-TSS | (NM_004686) |
| chr8 | 17270937 | P_DEgenes | ExpMeth | | | 235 | promoter-TSS | (NM_004686) |
| chr8 | 53045604 | P_DEgenes | ExpMeth | | | 236 | intron | (NM_001352827, |
| chr8 | 53045678 | P_DEgenes | ExpMeth | | | 237 | intron | (NM_001352827, |
| chr8 | 53050272 | P_DEgenes | ExpMeth | | | 238 | intron | (NM_001352827, |
| chr8 | 53130239 | P_DEgenes | ExpMeth | | | 239 | 5′ | UTR |
| chr8 | 5313028 | P_DEgenes | ExpMeth | | | 240 | 5′ | UTR |
| chr8 | 53130282 | P_DEgenes | ExpMeth | | | 241 | 5′ | UTR |
| chr8 | 53134555 | P_DEgenes | ExpMeth | | | 242 | intron | (NM_001352838, |
| chr8 | 53159124 | P_DEgenes | ExpMeth | | | 243 | non-coding | (NR_148195, |
| chr8 | 53160986 | P_DEgenes | ExpMeth | | | 244 | non-coding | (NR_148196, |
| chr8 | 53161872 | P_DEgenes | ExpMeth | | | 245 | promoter-TSS | (NM_001352856) |
| chr8 | 53166808 | P_DEgenes | ExpMeth | | | 246 | 5′ | UTR |
| chr8 | 53322203 | P_DEgenes | ExpMeth | | | 247 | promoter-TSS | (NM_001352883) |
| chr8 | 53322209 | P_DEgenes | ExpMeth | | | 248 | promoter-TSS | (NM_001352883) |
| chr8 | 53322216 | P_DEgenes | ExpMeth | | | 249 | promoter-TSS | (NM_001352883) |
| chr8 | 53322232 | P_DEgenes | ExpMeth | | | 250 | promoter-TSS | (NM_001352883) |
| chr8 | 53322252 | P_DEgenes | ExpMeth | | | 251 | promoter-TSS | (NM_001352826) |
| chr8 | 53322265 | P_DEgenes | ExpMeth | | | 252 | promoter-TSS | (NM_001352826) |
| chr8 | 53322339 | P_DEgenes | ExpMeth | | | 253 | promoter-TSS | (NM_001352826) |
| chr8 | 53322342 | P_DEgenes | ExpMeth | | | 254 | promoter-TSS | (NM_001352826) |
| chr8 | 53322405 | P_DEgenes | ExpMeth | | | 255 | promoter-TSS | (NM_001352826) |
| chr8 | 53373419 | P_DEgenes | ExpMeth | | | 256 | Intergenic | |
| chr8 | 104152838 | P_DEgenes | ExpMeth | | | 257 | promoter-TSS | (NM_001024372) |
| chr8 | 104152841 | P_DEgenes | ExpMeth | | | 258 | promoter-TSS | (NM_001024372) |
| chr8 | 104152887 | P_DEgenes | ExpMeth | | | 259 | promoter-TSS | (NM_001024372) |
| chr8 | 104153026 | P_DEgenes | ExpMeth | | | 260 | TTS | (NR_145429) |
| chr8 | 104165267 | P_DEgenes | ExpMeth | | | 261 | intron | (NM_001024372, |
| chr8 | 104220320 | P_DEgenes | ExpMeth | | | 262 | intron | (NM_001364874, |
| chr8 | 104225459 | P_DEgenes | ExpMeth | | | 263 | intron | (NM_001364874, |
| chr8 | 104225539 | P_DEgenes | ExpMeth | | | 264 | intron | (NM_001364874, |
| chr10 | 69597717 | P_DEgenes | ExpMeth | | | 265 | 5′ | UTR |
| chr10 | 69597721 | P_DEgenes | ExpMeth | | | 266 | 5′ | UTR |
| chr10 | 69597754 | P_DEgenes | ExpMeth | | | 267 | promoter-TSS | (NM_021800) |
| chr10 | 69597822 | P_DEgenes | ExpMeth | | | 268 | promoter-TSS | (NM_021800) |
| chr10 | 69597824 | P_DEgenes | ExpMeth | | | 269 | promoter-TSS | (NM_021800) |
| chr10 | 88428106 | P_DEgenes | ExpMeth | | | 270 | promoter-TSS | (NM_001080116) |
| chr10 | 88428270 | P_DEgenes | ExpMeth | | | 271 | promoter-TSS | (NM_001080116) |
| chr10 | 88428326 | P_DEgenes | ExpMeth | | | 272 | 5′ | UTR |
| chr10 | 88428328 | P_DEgenes | ExpMeth | | | 273 | 5′ | UTR |
| chr10 | 88428349 | P_DEgenes | ExpMeth | | | 274 | exon | (NM_001080116, |
| chr10 | 88478387 | P_DEgenes | ExpMeth | | | 275 | exon | (NM_007078, |
| chr10 | 92680933 | P_DEgenes | ExpMeth | | | 276 | promoter-TSS | (NM_014391) |
| chr11 | 60869767 | P_DEgenes | ExpMeth | | | 277 | promoter-TSS | (NM_014207) |
| chr11 | 60869881 | P_DEgenes | ExpMeth | | | 278 | promoter-TSS | (NM_014207) |
| chr11 | 67350966 | P_DEgenes | ExpMeth | | | 279 | promoter-TSS | (NM_000852) |
| chr11 | 67351113 | P_DEgenes | ExpMeth | | | 280 | 5′ | UTR |
| chr11 | 67351193 | P_DEgenes | ExpMeth | | | 281 | 5′ | UTR |
| chr11 | 67351495 | P_DEgenes | ExpMeth | | | 282 | intron | (NM_000852, |
| chr11 | 67351496 | P_DEgenes | ExpMeth | | | 283 | intron | (NM_000852, |
| chr11 | 67351505 | P_DEgenes | ExpMeth | | | 284 | exon | (NM_000852, |
| chr11 | 67351826 | P_DEgenes | ExpMeth | | | 285 | intron | (NM_000852, |
| chr11 | 67352166 | P_DEgenes | ExpMeth | | | 286 | intron | (NM_000852, |
| chr11 | 67353475 | P_DEgenes | ExpMeth | | | 287 | exon | (NM_000852, |
| chr11 | 87908501 | P_DEgenes | ExpMeth | | | 288 | promoter-TSS | (NM_022337) |
| chr11 | 87908502 | P_DEgenes | ExpMeth | | | 289 | promoter-TSS | (NM_022337) |
| chr11 | 87908535 | P_DEgenes | ExpMeth | | | 290 | promoter-TSS | (NM_022337) |
| chr11 | 112831897 | P_DEgenes | ExpMeth | | | 291 | promoter-TSS | (NM_001242608) |
| chr11 | 112831990 | P_DEgenes | ExpMeth | | | 292 | promoter-TSS | (NM_001242608) |
| chr11 | 112832024 | P_DEgenes | ExpMeth | | | 293 | promoter-TSS | (NM_001242608) |
| chr11 | 112832047 | P_DEgenes | ExpMeth | | | 294 | promoter-TSS | (NM_001242608) |
| chr11 | 112832057 | P_DEgenes | ExpMeth | | | 295 | promoter-TSS | (NM_001242608) |
| chr11 | 112832058 | P_DEgenes | ExpMeth | | | 296 | promoter-TSS | (NM_001242608) |
| chr11 | 112832059 | P_DEgenes | ExpMeth | | | 297 | promoter-TSS | (NM_001242608) |
| chr11 | 112832061 | P_DEgenes | ExpMeth | | | 298 | promoter-TSS | (NM_001242608) |
| chr11 | 112832102 | P_DEgenes | ExpMeth | | | 299 | promoter-TSS | (NM_001242608) |
| chr11 | 113001316 | P_DEgenes | ExpMeth | | | 300 | intron | (NM_181351, |
| chr11 | 113003550 | P_DEgenes | ExpMeth | | | 301 | intron | (NM_181351, |
| chr11 | 113067815 | P_DEgenes | ExpMeth | | | 302 | intron | (NM_181351, |
| chr11 | 113072979 | P_DEgenes | ExpMeth | | | 303 | intron | (NM_181351, |
| chr11 | 113072998 | P_DEgenes | ExpMeth | | | 304 | intron | (NM_181351, |
| chr11 | 113102002 | P_DEgenes | ExpMeth | | | 305 | intron | (NM_001242607, |
| chr11 | 113102884 | P_DEgenes | ExpMeth | | | 306 | exon | (NM_001242608, |
| chr11 | 113102947 | P_DEgenes | ExpMeth | | | 307 | exon | (NM_001242608, |
| chr11 | 113103415 | P_DEgenes | ExpMeth | | | 308 | exon | (NM_001242608, |
| chr11 | 113103417 | P_DEgenes | ExpMeth | | | 309 | exon | (NM_001242608, |
| chr11 | 113103766 | P_DEgenes | ExpMeth | | | 310 | intron | (NM_181351, |
| chr11 | 113103880 | P_DEgenes | ExpMeth | | | 311 | exon | (NM_001242608, |
| chr11 | 113111129 | P_DEgenes | ExpMeth | | | 312 | intron | (NM_001076682, |
| chr11 | 113116730 | P_DEgenes | ExpMeth | | | 313 | intron | (NM_001076682, |
| chr11 | 113126572 | P_DEgenes | ExpMeth | | | 314 | exon | (NM_001242608, |
| chr11 | 113130713 | P_DEgenes | ExpMeth | | | 315 | intron | (NM_181351, |
| chr11 | 113130880 | P_DEgenes | ExpMeth | | | 316 | exon | (NM_001242608, |
| chr11 | 113140591 | P_DEgenes | ExpMeth | | | 317 | intron | (NR_034101, |
| chr11 | 113143642 | P_DEgenes | ExpMeth | | | 318 | intron | (NM_181351, |
| chr11 | 133938720 | P_DEgenes | ExpMeth | | | 319 | promoter-TSS | (NM_001205329) |
| chr11 | 133938866 | P_DEgenes | ExpMeth | | | 320 | promoter-TSS | (NM_032801) |
| chr11 | 133938880 | P_DEgenes | ExpMeth | | | 321 | promoter-TSS | (NM_032801) |
| chr11 | 133996134 | P_DEgenes | ExpMeth | | | 322 | intron | (NM_032801, |
| chr11 | 134009435 | P_DEgenes | ExpMeth | | | 323 | intron | (NM_032801, |
| chr11 | 134017593 | P_DEgenes | ExpMeth | | | 324 | intron | (NM_032801, |
| chr12 | 7022809 | P_DEgenes | ExpMeth | | | 325 | promoter-TSS | (NM_001975) |
| chr12 | 7023391 | P_DEgenes | ExpMeth | | | 326 | promoter-TSS | (NM_001975) |
| chr12 | 7023514 | P_DEgenes | ExpMeth | | | 327 | promoter-TSS | (NM_001975) |
| chr12 | 7023602 | P_DEgenes | ExpMeth | | | 328 | promoter-TSS | (NM_001975) |
| chr12 | 7023621 | P_DEgenes | ExpMeth | | | 329 | promoter-TSS | (NM_001975) |
| chr12 | 7023653 | P_DEgenes | ExpMeth | | | 330 | promoter-TSS | (NM_001975) |
| chr12 | 7023658 | P_DEgenes | ExpMeth | | | 331 | promoter-TSS | (NM_001975) |
| chr12 | 7023661 | P_DEgenes | ExpMeth | | | 332 | promoter-TSS | (NM_001975) |
| chr12 | 7023674 | P_DEgenes | ExpMeth | | | 333 | promoter-TSS | (NM_001975) |
| chr12 | 7024271 | P_DEgenes | ExpMeth | | | 334 | TTS | (NM_001135217) |
| chr12 | 7026393 | P_DEgenes | ExpMeth | | | 335 | intron | (NM_001975, |
| chr12 | 7028638 | P_DEgenes | ExpMeth | | | 336 | exon | (NM_001975, |
| chr12 | 7030644 | P_DEgenes | ExpMeth | | | 337 | exon | (NM_001975, |
| chr12 | 7031052 | P_DEgenes | ExpMeth | | | 338 | intron | (NM_001975, |
| chr12 | 24983162 | P_DEgenes | ExpMeth | | | 339 | intron | (NM_001178092, |
| chr12 | 25055130 | P_DEgenes | ExpMeth | | | 340 | promoter-TSS | (NM_001178094) |
| chr12 | 25055222 | P_DEgenes | ExpMeth | | | 341 | promoter-TSS | (NM_001178094) |
| chr12 | 25055909 | P_DEgenes | ExpMeth | | | 342 | promoter-TSS | (NM_001178094) |
| chr12 | 25101877 | P_DEgenes | ExpMeth | | | 343 | promoter-TSS | (NM_001178091) |
| chr12 | 25101883 | P_DEgenes | ExpMeth | | | 344 | promoter-TSS | (NM_001178091) |
| chr12 | 25101895 | P_DEgenes | ExpMeth | | | 345 | promoter-TSS | (NM_001178091) |
| chr12 | 25101920 | P_DEgenes | ExpMeth | | | 346 | promoter-TSS | (NM_001178091) |
| chr12 | 25102293 | P_DEgenes | ExpMeth | | | 347 | promoter-TSS | (NM_001178091) |
| chr12 | 25173836 | P_DEgenes | ExpMeth | | | 348 | intron | (NM_001321724, |
| chr12 | 25186593 | P_DEgenes | ExpMeth | | | 349 | intron | (NM_001321724, |
| chr12 | 25188314 | P_DEgenes | ExpMeth | | | 350 | intron | (NM_001321724, |
| chr12 | 25205089 | P_DEgenes | ExpMeth | | | 351 | promoter-TSS | (NM_001366542) |
| chr12 | 25205105 | P_DEgenes | ExpMeth | | | 352 | promoter-TSS | (NM_001366542) |
| chr12 | 25205106 | P_DEgenes | ExpMeth | | | 353 | promoter-TSS | (NM_001366542) |
| chr12 | 25205346 | P_DEgenes | ExpMeth | | | 354 | promoter-TSS | (NM_001204126) |
| chr12 | 25205421 | P_DEgenes | ExpMeth | | | 355 | promoter-TSS | (NM_001204126) |
| chr12 | 25205476 | P_DEgenes | ExpMeth | | | 356 | promoter-TSS | (NM_001204126) |
| chr12 | 25205499 | P_DEgenes | ExpMeth | | | 357 | promoter-TSS | (NM_001204126) |
| chr12 | 25205534 | P_DEgenes | ExpMeth | | | 358 | promoter-TSS | (NM_001204126) |
| chr12 | 25205574 | P_DEgenes | ExpMeth | | | 359 | promoter-TSS | (NM_001204126) |
| chr12 | 25205779 | P_DEgenes | ExpMeth | | | 360 | TTS | (NM_001321724) |
| chr12 | 25228574 | P_DEgenes | ExpMeth | | | 361 | intron | (NR_159367, |
| chr12 | 25242608 | P_DEgenes | ExpMeth | | | 362 | exon | (NM_001366542, |
| chr12 | 25249767 | P_DEgenes | ExpMeth | | | 363 | exon | (NM_001366542, |
| chr12 | 25249792 | P_DEgenes | ExpMeth | | | 364 | exon | (NM_001366542, |
| chr12 | 25257163 | P_DEgenes | ExpMeth | | | 365 | intron | (NR_159367, |
| chr12 | 66582559 | P_DEgenes | ExpMeth | | | 366 | promoter-TSS | (NM_001142523) |
| chr12 | 66582875 | P_DEgenes | ExpMeth | | | 367 | promoter-TSS | (NM_001142523) |
| chr12 | 66582937 | P_DEgenes | ExpMeth | | | 368 | promoter-TSS | (NM_001142523) |
| chr12 | 70082891 | P_DEgenes | ExpMeth | | | 369 | promoter-TSS | (NM_152439) |
| chr12 | 70082956 | P_DEgenes | ExpMeth | | | 370 | promoter-TSS | (NM_152439) |
| chr12 | 70087752 | P_DEgenes | ExpMeth | | | 371 | intron | (NM_001282615, |
| chr12 | 70092965 | P_DEgenes | ExpMeth | | | 372 | promoter-TSS | (NM_001282616) |
| chr12 | 70093015 | P_DEgenes | ExpMeth | | | 373 | promoter-TSS | (NM_001282614) |
| chr12 | 70093024 | P_DEgenes | ExpMeth | | | 374 | promoter-TSS | (NM_001282614) |
| chr12 | 70093031 | P_DEgenes | ExpMeth | | | 375 | promoter-TSS | (NM_001282614) |
| chr12 | 70093041 | P_DEgenes | ExpMeth | | | 376 | promoter-TSS | (NM_001282614) |
| chr12 | 70093044 | P_DEgenes | ExpMeth | | | 377 | promoter-TSS | (NM_001282614) |
| chr12 | 70093075 | P_DEgenes | ExpMeth | | | 378 | promoter-TSS | (NM_001282614) |
| chr12 | 70093096 | P_DEgenes | ExpMeth | | | 379 | promoter-TSS | (NM_001282614) |
| chr12 | 70093156 | P_DEgenes | ExpMeth | | | 380 | promoter-TSS | (NM_001282614) |
| chr12 | 103351364 | P_DEgenes | ExpMeth | | | 381 | promoter-TSS | (NM_004316) |
| chr13 | 102527544 | P_DEgenes | ExpMeth | | | 382 | exon | (NM_001321933, |
| chr13 | 102568895 | P_DEgenes | ExpMeth | | | 383 | promoter-TSS | (NM_004115) |
| chr13 | 103054024 | P_DEgenes | ExpMeth | | | 384 | promoter-TSS | (NM_175929) |
| chr14 | 21994247 | P_DEgenes | ExpMeth | | | 385 | non-coding | (NR_111967, |
| chr14 | 21994261 | P_DEgenes | ExpMeth | | | 386 | non-coding | (NR_111967, |
| chr14 | 21994534 | P_DEgenes | ExpMeth | | | 387 | promoter-TSS | (NR_111967) |
| chr14 | 22004936 | P_DEgenes | ExpMeth | | | 388 | non-coding | (NR_111966, |
| chr14 | 22004968 | P_DEgenes | ExpMeth | | | 389 | non-coding | (NR_111966, |
| chr14 | 22005138 | P_DEgenes | ExpMeth | | | 390 | promoter-TSS | (NM_001291446) |
| chr14 | 22005250 | P_DEgenes | ExpMeth | | | 391 | promoter-TSS | (NR_111966) |
| chr14 | 51448929 | P_DEgenes | ExpMeth | | | 392 | intron | (NM_015163, |
| chr14 | 51453070 | P_DEgenes | ExpMeth | | | 393 | intron | (NM_015163, |
| chr14 | 51454994 | P_DEgenes | ExpMeth | | | 394 | intron | (NM_015163, |
| chr14 | 51561694 | P_DEgenes | ExpMeth | | | 395 | 5′ | UTR |
| chr14 | 51561947 | P_DEgenes | ExpMeth | | | 396 | 5′ | UTR |
| chr14 | 51562679 | P_DEgenes | ExpMeth | | | 397 | promoter-TSS | (NM_015163) |
| chr14 | 75988668 | P_DEgenes | ExpMeth | | | 398 | promoter-TSS | (NM_006399) |
| chr14 | 75988698 | P_DEgenes | ExpMeth | | | 399 | promoter-TSS | (NM_006399) |
| chr14 | 75988803 | P_DEgenes | ExpMeth | | | 400 | promoter-TSS | (NM_006399) |
| chr14 | 93389325 | P_DEgenes | ExpMeth | | | 401 | promoter-TSS | (NM_001275) |
| chr14 | 93389402 | P_DEgenes | ExpMeth | | | 402 | promoter-TSS | (NM_001275) |
| chr14 | 93389421 | P_DEgenes | ExpMeth | | | 403 | promoter-TSS | (NM_001275) |
| chr14 | 93389608 | P_DEgenes | ExpMeth | | | 404 | exon | (NM_001275, |
| chr14 | 93392346 | P_DEgenes | ExpMeth | | | 405 | intron | (NM_001301690, |
| chr14 | 93397922 | P_DEgenes | ExpMeth | | | 406 | exon | (NM_001275, |
| chr15 | 74465912 | P_DEgenes | ExpMeth | | | 407 | promoter-TSS | (NM_005545) |
| chr15 | 74466371 | P_DEgenes | ExpMeth | | | 408 | promoter-TSS | (NM_201526) |
| chr15 | 74466644 | P_DEgenes | ExpMeth | | | 409 | promoter-TSS | (NM_201526) |
| chr15 | 74466742 | P_DEgenes | ExpMeth | | | 410 | promoter-TSS | (NM_201526) |
| chr15 | 77285600 | P_DEgenes | ExpMeth | | | 411 | Intergenic | |
| chr15 | 77286921 | P_DEgenes | ExpMeth | | | 412 | promoter-TSS | (NM_001321136) |
| chr15 | 77287326 | P_DEgenes | ExpMeth | | | 413 | promoter-TSS | (NM_001321136) |
| chr15 | 77287363 | P_DEgenes | ExpMeth | | | 414 | promoter-TSS | (NM_001321136) |
| chr15 | 77287365 | P_DEgenes | ExpMeth | | | 415 | promoter-TSS | (NM_001321136) |
| chr15 | 77287367 | P_DEgenes | ExpMeth | | | 416 | promoter-TSS | (NM_001321136) |
| chr15 | 77287372 | P_DEgenes | ExpMeth | | | 417 | promoter-TSS | (NM_001321136) |
| chr15 | 77287413 | P_DEgenes | ExpMeth | | | 418 | promoter-TSS | (NM_001321136) |
| chr15 | 77287603 | P_DEgenes | ExpMeth | | | 419 | non-coding | (NR_135552, |
| chr15 | 77287626 | P_DEgenes | ExpMeth | | | 420 | non-coding | (NR_135552, |
| chr15 | 77287653 | P_DEgenes | ExpMeth | | | 421 | exon | (NM_001321137, |
| chr15 | 77287660 | P_DEgenes | ExpMeth | | | 422 | exon | (NM_001321137, |
| chr15 | 77287815 | P_DEgenes | ExpMeth | | | 423 | exon | (NM_001321135, |
| chr15 | 77308052 | P_DEgenes | ExpMeth | | | 424 | intron | (NM_001321136, |
| chr15 | 77310699 | P_DEgenes | ExpMeth | | | 425 | exon | (NM_001321136, |
| chr15 | 77319966 | P_DEgenes | ExpMeth | | | 426 | intron | (NR_135552, |
| chr15 | 77320584 | P_DEgenes | ExpMeth | | | 427 | intron | (NR_135552, |
| chr15 | 77324558 | P_DEgenes | ExpMeth | | | 428 | exon | (NM_001321136, |
| chr15 | 77327935 | P_DEgenes | ExpMeth | | | 429 | intron | (NR_135552, |
| chr16 | 29674500 | P_DEgenes | ExpMeth | | | 430 | promoter-TSS | (NM_003123) |
| chr16 | 29690229 | P_DEgenes | ExpMeth | | | 431 | promoter-TSS | (NR_134536) |
| chr16 | 29690258 | P_DEgenes | ExpMeth | | | 432 | promoter-TSS | (NR_134536) |
| chr16 | 29690403 | P_DEgenes | ExpMeth | | | 433 | promoter-TSS | (NR_134536) |
| chr16 | 29707018 | P_DEgenes | ExpMeth | | | 434 | intron | (NM_014298, |
| chr16 | 31146861 | P_DEgenes | ExpMeth | | | 435 | promoter-TSS | (NM_002773) |
| chr16 | 31146924 | P_DEgenes | ExpMeth | | | 436 | promoter-TSS | (NM_002773) |
| chr16 | 31146948 | P_DEgenes | ExpMeth | | | 437 | promoter-TSS | (NM_002773) |
| chr16 | 31146983 | P_DEgenes | ExpMeth | | | 438 | promoter-TSS | (NM_002773) |
| chr16 | 74710849 | P_DEgenes | ExpMeth | | | 439 | intron | (NM_152649, |
| chr16 | 74712211 | P_DEgenes | ExpMeth | | | 440 | intron | (NM_152649, |
| chr16 | 74712746 | P_DEgenes | ExpMeth | | | 441 | exon | (NM_152649, |
| chr16 | 74725267 | P_DEgenes | ExpMeth | | | 442 | intron | (NM_152649, |
| chr16 | 74734588 | P_DEgenes | ExpMeth | | | 443 | promoter-TSS | (NM_001142497) |
| chr16 | 74734607 | P_DEgenes | ExpMeth | | | 444 | promoter-TSS | (NM_001142497) |
| chr16 | 74734712 | P_DEgenes | ExpMeth | | | 445 | promoter-TSS | (NM_001142497) |
| chr16 | 74734758 | P_DEgenes | ExpMeth | | | 446 | promoter-TSS | (NM_001142497) |
| chr16 | 88713133 | P_DEgenes | ExpMeth | | | 447 | exon | (NM_000101, |
| chr16 | 88716622 | P_DEgenes | ExpMeth | | | 448 | intron | (NM_000101, |
| chr16 | 88717296 | P_DEgenes | ExpMeth | | | 449 | promoter-TSS | (NM_000101) |
| chr16 | 88717323 | P_DEgenes | ExpMeth | | | 450 | promoter-TSS | (NM_000101) |
| chr16 | 88717338 | P_DEgenes | ExpMeth | | | 451 | promoter-TSS | (NM_000101) |
| chr16 | 88717339 | P_DEgenes | ExpMeth | | | 452 | promoter-TSS | (NM_000101) |
| chr16 | 88717343 | P_DEgenes | ExpMeth | | | 453 | promoter-TSS | (NM_000101) |
| chr16 | 88717349 | P_DEgenes | ExpMeth | | | 454 | promoter-TSS | (NM_000101) |
| chr16 | 88717362 | P_DEgenes | ExpMeth | | | 455 | promoter-TSS | (NM_000101) |
| chr16 | 88717460 | P_DEgenes | ExpMeth | | | 456 | promoter-TSS | (NM_000101) |
| chr17 | 1665153 | P_DEgenes | ExpMeth | | | 457 | promoter-TSS | (NM_002615) |
| chr17 | 1665223 | P_DEgenes | ExpMeth | | | 458 | promoter-TSS | (NM_002615) |
| chr17 | 1665255 | P_DEgenes | ExpMeth | | | 459 | promoter-TSS | (NM_002615) |
| chr17 | 1665262 | P_DEgenes | ExpMeth | | | 460 | promoter-TSS | (NM_002615) |
| chr17 | 1665306 | P_DEgenes | ExpMeth | | | 461 | promoter-TSS | (NM_002615) |
| chr17 | 1666023 | P_DEgenes | ExpMeth | | | 462 | promoter-TSS | (NM_001329903) |
| chr17 | 1669468 | P_DEgenes | ExpMeth | | | 463 | intron | (NM_001329903, |
| chr17 | 1673720 | P_DEgenes | ExpMeth | | | 464 | intron | (NM_001329903, |
| chr17 | 1674913 | P_DEgenes | ExpMeth | | | 465 | promoter-TSS | (NM_001329905) |
| chr17 | 1679644 | P_DEgenes | ExpMeth | | | 466 | intron | (NM_001329903, |
| chr17 | 27283357 | P_DEgenes | ExpMeth | | | 467 | exon | (NM_178860, |
| chr17 | 27284731 | P_DEgenes | ExpMeth | | | 468 | intron | (NM_001098635, |
| chr17 | 27287891 | P_DEgenes | ExpMeth | | | 469 | exon | (NM_178860, |
| chr17 | 27308510 | P_DEgenes | ExpMeth | | | 470 | exon | (NM_178860, |
| chr17 | 27308790 | P_DEgenes | ExpMeth | | | 471 | exon | (NM_178860, |
| chr17 | 27332831 | P_DEgenes | ExpMeth | | | 472 | 5′ | UTR |
| chr17 | 27332981 | P_DEgenes | ExpMeth | | | 473 | promoter-TSS | (NM_178860) |
| chr17 | 27333063 | P_DEgenes | ExpMeth | | | 474 | promoter-TSS | (NM_178860) |
| chr17 | 27333216 | P_DEgenes | ExpMeth | | | 475 | promoter-TSS | (NM_178860) |
| chr17 | 27333358 | P_DEgenes | ExpMeth | | | 476 | promoter-TSS | (NM_178860) |
| chr17 | 62007774 | P_DEgenes | ExpMeth | | | 477 | intron | (NM_001039933, |
| chr17 | 62009521 | P_DEgenes | ExpMeth | | | 478 | promoter-TSS | (NM_000626) |
| chr17 | 62009583 | P_DEgenes | ExpMeth | | | 479 | promoter-TSS | (NM_000626) |
| chr17 | 62009591 | P_DEgenes | ExpMeth | | | 480 | promoter-TSS | (NM_000626) |
| chr17 | 62009596 | P_DEgenes | ExpMeth | | | 481 | promoter-TSS | (NM_000626) |
| chr17 | 62009614 | P_DEgenes | ExpMeth | | | 482 | promoter-TSS | (NM_000626) |
| chr17 | 76970778 | P_DEgenes | ExpMeth | | | 483 | exon | (NM_005567, |
| chr17 | 76970979 | P_DEgenes | ExpMeth | | | 484 | intron | (NM_005567, |
| chr17 | 76972138 | P_DEgenes | ExpMeth | | | 485 | exon | (NM_005567, |
| chr17 | 76973198 | P_DEgenes | ExpMeth | | | 486 | intron | (NM_005567, |
| chr17 | 76973305 | P_DEgenes | ExpMeth | | | 487 | intron | (NM_005567, |
| chr17 | 76975825 | P_DEgenes | ExpMeth | | | 488 | promoter-TSS | (NM_005567) |
| chr17 | 76975857 | P_DEgenes | ExpMeth | | | 489 | promoter-TSS | (NM_005567) |
| chr17 | 76975873 | P_DEgenes | ExpMeth | | | 490 | promoter-TSS | (NM_005567) |
| chr17 | 76975890 | P_DEgenes | ExpMeth | | | 491 | promoter-TSS | (NM_005567) |
| chr17 | 76975893 | P_DEgenes | ExpMeth | | | 492 | promoter-TSS | (NM_005567) |
| chr17 | 76975900 | P_DEgenes | ExpMeth | | | 493 | promoter-TSS | (NM_005567) |
| chr17 | 76975905 | P_DEgenes | ExpMeth | | | 494 | promoter-TSS | (NM_005567) |
| chr17 | 76975909 | P_DEgenes | ExpMeth | | | 495 | promoter-TSS | (NM_005567) |
| chr17 | 76975917 | P_DEgenes | ExpMeth | | | 496 | promoter-TSS | (NM_005567) |
| chr17 | 76975929 | P_DEgenes | ExpMeth | | | 497 | promoter-TSS | (NM_005567) |
| chr17 | 76976091 | P_DEgenes | ExpMeth | | | 498 | promoter-TSS | (NM_005567) |
| chr18 | 25616449 | P_DEgenes | ExpMeth | | | 499 | promoter-TSS | (NM_001308176) |
| chr18 | 25625460 | P_DEgenes | ExpMeth | | | 500 | intron | (NM_001792, |
| chr18 | 25677093 | P_DEgenes | ExpMeth | | | 501 | intron | (NM_001792, |
| chr18 | 25739161 | P_DEgenes | ExpMeth | | | 502 | intron | (NM_001792, |
| chr18 | 25757310 | P_DEgenes | ExpMeth | | | 503 | promoter-TSS | (NM_001792) |
| chr18 | 31158479 | P_DEgenes | ExpMeth | | | 504 | 5′ | UTR |
| chr18 | 31158496 | P_DEgenes | ExpMeth | | | 505 | exon | (NM_030632, |
| chr18 | 31158505 | P_DEgenes | ExpMeth | | | 506 | exon | (NM_030632, |
| chr18 | 31158508 | P_DEgenes | ExpMeth | | | 507 | exon | (NM_030632, |
| chr18 | 31158530 | P_DEgenes | ExpMeth | | | 508 | exon | (NM_030632, |
| chr18 | 31158635 | P_DEgenes | ExpMeth | | | 509 | intron | (NM_030632, |
| chr18 | 31158746 | P_DEgenes | ExpMeth | | | 510 | intron | (NM_030632, |
| chr18 | 31185450 | P_DEgenes | ExpMeth | | | 511 | intron | (NM_030632, |
| chr18 | 31185451 | P_DEgenes | ExpMeth | | | 512 | intron | (NM_030632, |
| chr18 | 31185458 | P_DEgenes | ExpMeth | | | 513 | intron | (NM_030632, |
| chr18 | 31185463 | P_DEgenes | ExpMeth | | | 514 | intron | (NM_030632, |
| chr18 | 31185472 | P_DEgenes | ExpMeth | | | 515 | intron | (NM_030632, |
| chr18 | 31185490 | P_DEgenes | ExpMeth | | | 516 | intron | (NM_030632, |
| chr18 | 31187471 | P_DEgenes | ExpMeth | | | 517 | exon | (NM_030632, |
| chr19 | 39989435 | P_DEgenes | ExpMeth | | | 518 | promoter-TSS | (NM_016941) |
| chr19 | 39989507 | P_DEgenes | ExpMeth | | | 519 | promoter-TSS | (NM_016941) |
| chr19 | 39989508 | P_DEgenes | ExpMeth | | | 520 | promoter-TSS | (NM_016941) |
| chr19 | 46916741 | P_DEgenes | ExpMeth | | | 521 | promoter-TSS | (NM_032040) |
| chr19 | 51358071 | P_DEgenes | ExpMeth | | | 522 | promoter-TSS | (NM_001030048) |
| chr19 | 51358077 | P_DEgenes | ExpMeth | | | 523 | promoter-TSS | (NM_001030048) |
| chr19 | 51358086 | P_DEgenes | ExpMeth | | | 524 | promoter-TSS | (NM_001030048) |
| chr19 | 51358091 | P_DEgenes | ExpMeth | | | 525 | promoter-TSS | (NM_001030048) |
| chr19 | 51358105 | P_DEgenes | ExpMeth | | | 526 | promoter-TSS | (NM_001030048) |
| chr19 | 51358112 | P_DEgenes | ExpMeth | | | 527 | promoter-TSS | (NM_001030048) |
| chr19 | 51358128 | P_DEgenes | ExpMeth | | | 528 | promoter-TSS | (NM_001030048) |
| chr19 | 51360547 | P_DEgenes | ExpMeth | | | 529 | intron | (NM_001030048, |
| chr20 | 3210322 | P_DEgenes | ExpMeth | | | 530 | exon | (NM_001174089, |
| chr20 | 3211106 | P_DEgenes | ExpMeth | | | 531 | exon | (NM_001174089, |
| chr20 | 3218273 | P_DEgenes | ExpMeth | | | 532 | promoter-TSS | (NM_032034) |
| chr20 | 3218712 | P_DEgenes | ExpMeth | | | 533 | promoter-TSS | (NM_032034) |
| chr20 | 3219259 | P_DEgenes | ExpMeth | | | 534 | promoter-TSS | (NM_032034) |
| chr20 | 3219736 | P_DEgenes | ExpMeth | | | 535 | promoter-TSS | (NM_001174089) |
| chr20 | 5891976 | P_DEgenes | ExpMeth | | | 536 | promoter-TSS | (NM_001819) |
| chr20 | 5892076 | P_DEgenes | ExpMeth | | | 537 | promoter-TSS | (NM_001819) |
| chr20 | 5896044 | P_DEgenes | ExpMeth | | | 538 | intron | (NM_001819, |
| chr20 | 20348665 | P_DEgenes | ExpMeth | | | 539 | promoter-TSS | (NM_002196) |
| chr20 | 22565001 | P_DEgenes | ExpMeth | | | 540 | promoter-TSS | (NM_021784) |
| chr21 | 22565993 | P_DEgenes | ExpMeth | | | 541 | promoter-TSS | (NM_153675) |
| chr21 | 42839929 | P_DEgenes | ExpMeth | | | 542 | intron | (NM_005656, |
| chr21 | 42840664 | P_DEgenes | ExpMeth | | | 543 | intron | (NM_005656, |
| chr21 | 42878431 | P_DEgenes | ExpMeth | | | 544 | intron | (NM_005656, |
| chr21 | 42879809 | P_DEgenes | ExpMeth | | | 545 | promoter-TSS | (NM_001135099) |
| chr21 | 42879892 | P_DEgenes | ExpMeth | | | 546 | promoter-TSS | (NM_001135099) |
| chr21 | 42879919 | P_DEgenes | ExpMeth | | | 547 | promoter-TSS | (NM_001135099) |
| chr21 | 42879941 | P_DEgenes | ExpMeth | | | 548 | promoter-TSS | (NM_001135099) |
| chr21 | 42879985 | P_DEgenes | ExpMeth | | | 549 | promoter-TSS | (NM_001135099) |
| chr21 | 42879986 | P_DEgenes | ExpMeth | | | 550 | promoter-TSS | (NM_001135099) |
| chr21 | 42902925 | P_DEgenes | ExpMeth | | | 551 | Intergenic | |
| chr21 | 42902943 | P_DEgenes | ExpMeth | | | 552 | Intergenic | |
| chrX | 49056535 | P_DEgenes | ExpMeth | | | 553 | promoter-TSS | (NM_003179) |
| chrX | 49056552 | P_DEgenes | ExpMeth | | | 554 | promoter-TSS | (NM_003179) |
| chrX | 49056554 | P_DEgenes | ExpMeth | | | 555 | promoter-TSS | (NM_003179) |
| chrX | 49056558 | P_DEgenes | ExpMeth | | | 556 | promoter-TSS | (NM_003179) |
| chrX | 49056618 | P_DEgenes | ExpMeth | | | 557 | promoter-TSS | (NM_003179) |
| chrX | 66764365 | P_DEgenes | ExpMeth | | | 558 | promoter-TSS | (NM_001348064) |
| chrX | 66764562 | P_DEgenes | ExpMeth | | | 559 | 5′ | UTR |
| chrX | 66764886 | P_DEgenes | ExpMeth | | | 560 | 5′ | UTR |
| chrX | 66788631 | P_DEgenes | ExpMeth | | | 561 | 5′ | UTR |
| chr1 | 202039153 | CpG_2020 | AUC_dBeta | −1 | | 1 | Intergenic | |
| chr1 | 241206098 | CpG_2020 | AUC_dBeta | 1 | | 2 | intron | (NM_001350116, |
| chr2 | 89530057 | CpG_2020 | AUC_dBeta | −1 | | 3 | Intergenic | |
| chr3 | 193490472 | CpG_2020 | AUC_dBeta | −1 | | 4 | Intergenic | |
| chr5 | 1548487 | CpG_2020 | AUC_dBeta | 1 | | 5 | Intergenic | |
| chr5 | 36521979 | CpG_2020 | AUC_dBeta | −1 | | 6 | Intergenic | |
| chr5 | 39390555 | CpG_2020 | AUC_dBeta | −1 | | 7 | exon | (NM_001244871, |
| chr5 | 71146894 | CpG_2020 | AUC_dBeta | 1 | | 8 | Intergenic | |
| chr5 | 142194123 | CpG_2020 | AUC_dBeta | 1 | | 9 | intron | (NM_001349547, |
| chr5 | 168573593 | CpG_2020 | AUC_dBeta | −1 | | 10 | intron | (NM_001271946, |
| chr7 | 2006579 | CpG_2020 | AUC_dBeta | −1 | | 11 | intron | (NM_001304523, |
| chr7 | 7799018 | CpG_2020 | AUC_dBeta | 1 | | 12 | intron | (NM_001302350, |
| chr7 | 127841628 | CpG_2020 | AUC_dBeta | 1 | | 13 | Intergenic | |
| chr7 | 129912398 | CpG_2020 | AUC_dBeta | −1 | | 14 | intron | (NM_001869, |
| chr8 | 53499560 | CpG_2020 | AUC_dBeta | 1 | | 15 | Intergenic | |
| chr8 | 61990390 | CpG_2020 | AUC_dBeta | −1 | | 16 | Intergenic | |
| chr8 | 69087227 | CpG_2020 | AUC_dBeta | −1 | | 17 | intron | (NM_024870, |
| chr8 | 73479990 | CpG_2020 | AUC_dBeta | 1 | | 18 | exon | (NM_004770, |
| chr8 | 73849054 | CpG_2020 | AUC_dBeta | 1 | | 19 | exon | (NM_004770, |
| chr8 | 133070961 | CpG_2020 | AUC_dBeta | −1 | | 20 | intron | (NM_001080399, |
| chr8 | 140727998 | CpG_2020 | AUC_dBeta | 1 | | 21 | Intergenic | |
| chr8 | 141458926 | CpG_2020 | AUC_dBeta | −1 | | 22 | intron | (NM_001160372, |
| chr8 | 141994084 | CpG_2020 | AUC_dBeta | 1 | | 23 | | UTR |
| chr8 | 142367954 | CpG_2020 | AUC_dBeta | 1 | | 24 | exon | (NM_005293, |
| chr8 | 143395944 | CpG_2020 | AUC_dBeta | 1 | | 25 | intron | (NM_001363740, |
| chr9 | 36748433 | CpG_2020 | AUC_dBeta | 1 | | 26 | Intergenic | |
| chr9 | 38437314 | CpG_2020 | AUC_dBeta | 1 | | 27 | Intergenic | |
| chr9 | 93792388 | CpG_2020 | AUC_dBeta | 1 | | 28 | Intergenic | |
| chr9 | 133769512 | CpG_2020 | AUC_dBeta | 1 | | 29 | intron | (NM_198180, |
| chr9 | 134913164 | CpG_2020 | AUC_dBeta | −1 | | 30 | intron | (NM_004269, |
| chr9 | 138719304 | CpG_2020 | AUC_dBeta | 1 | | 31 | exon | (NM_015447, |
| chr14 | 100039425 | CpG_2020 | AUC_dBeta | −1 | | 32 | intron | (NM_001144995, |
| chr18 | 8660098 | CpG_2020 | AUC_dBeta | −1 | | 33 | Intergenic | |
| chr18 | 14917016 | CpG_2020 | AUC_dBeta | −1 | | 34 | Intergenic | |
| chr18 | 25442246 | CpG_2020 | AUC_dBeta | −1 | | 35 | Intergenic | |
| chr18 | 47258089 | CpG_2020 | AUC_dBeta | 1 | | 36 | Intergenic | |
| chr20 | 20421462 | CpG_2020 | AUC_dBeta | −1 | | 37 | intron | (NM_020343, |
| chr20 | 21563921 | CpG_2020 | AUC_dBeta | −1 | | 38 | intron | (NR_109880, |
| chr21 | 32546326 | CpG_2020 | AUC_dBeta | 1 | | 39 | intron | (NM_001353687, |
| chr21 | 46885392 | CpG_2020 | AUC_dBeta | −1 | | 40 | intron | (NM_130444, |

| | | | Distance | Nearest | | | |
|---|---|---|---|---|---|---|---|
| | start_hg19 | Detailed Annotation | to TSS | PromoterID | Entrez ID | Nearest Ensembl | Gene Name |
| chr1 | 32237823 | CpG | −8133 | NM_001294336 | 576 | ENSG00000121753 | ADGRB2 |
| chr1 | 43814069 | intron (NM_005373,ntron 8 of 11) | −10388 | NM_001255 | 991 | ENSG00000117399 | CDC20 |
| chr1 | 151345023 | promoter-TSS (NM_001258288) | −5 | NM_001258288 | 8991 | ENSG00000143416 | SELENBP1 |
| chr1 | 151810787 | non-coding (NR_024237, exon 1 of 5) | 143 | NR_024237 | 100132111 | | C2CD4D-AS1 |
| chr2 | 19561227 | CpG | −3134 | NM_145260 | 130497 | ENSG00000143867 | OSR1 |
| chr2 | 63283706 | non-coding (NR_130153, exon 5 of 5) | 6004 | NM_014562 | 5013 | ENSG00000115507 | OTX1 |
| chr2 | 177030050 | intron (NM_006898, intron 2 of 3) | 8154 | NM_006898 | 3232 | ENSG00000128652 | HOXD3 |
| chr2 | 201450223 | promoter-TSS (NM_001159) | −204 | NM_001159 | 316 | ENSG00000138356 | AOX1 |
| chr3 | 50378091 | promoter-TSS (NM_170714) | −61 | NM_001206957 | 11186 | ENSG00000068028 | RASSF1 |
| chr3 | 122296269 | promoter-TSS (NM_001113523) | 77 | NM_001113523 | 165631 | ENSG00000173200 | PARP15 |
| chr3 | 124860516 | CpG | 9652 | NR_049815 | 100847039 | ENSG00000264986 | MIR5092 |
| chr5 | 958708 | CpG | 38480 | NR_104615 | 100506688 | ENSG00000215246 | LOC100506688 |
| chr5 | 3187966 | CpG | 10271 | NR_104617 | 102467074 | ENSG00000249808 | LINC01377 |
| chr5 | 180230667 | promoter-TSS (NM_001364390) | −79 | NM_001114620 | 4245 | ENSG00000131446 | MGAT1 |
| chr6 | 13014771 | CpG | 1260 | NM_001322314 | 221692 | ENSG00000112137 | PHACTR1 |
| chr6 | 15505245 | intron (NM_004973, intron 9 of 17) | 157856 | NR_036448 | 84062 | ENSG00000047579 | DTNBP1 |
| chr6 | 26271093 | exon (NM_003534, exon 1 of 1) | 102 | NM_003534 | 8355 | ENSG00000273983 | HIST1H3G |
| chr7 | 73245339 | exon (NM_001305, exon 1 of 1) | 403 | NM_001305 | 1364 | ENSG00000189143 | CLDN4 |
| chr7 | 129425230 | CpG | −10777 | NR_029615 | 406959 | ENSG00000207691 | MIR183 |
| chr8 | 56852048 | intron (NM_002350, intron 1 of 12) | −36805 | NR_145767 | 109617001 | ENSG00000199405 | SNORA1B |
| chr8 | 99960398 | exon (NM_001286841, exon 2 of 5) | 3869 | NM_001142462 | 116039 | ENSG00000164920 | OSR2 |
| chr8 | 143625497 | 3′ UTR (NM_001702, exon 3 of 31) | 70058 | NM_015193 | 23237 | ENSG00000198576 | ARC |
| chr8 | 145104006 | promoter-TSS (NR_158554) | −66 | NR_158554 | 392275 | | SMPD5 |
| chr10 | 6297648 | Intergenic | 52935 | NM_001363545 | 5209 | ENSG00000170525 | PFKFB3 |
| chr10 | 22541895 | non-coding (NR_038921, exon 1 of 1) | 1069 | NR_038921 | 100130992 | | LOC100130992 |
| chr11 | 393838 | promoter-TSS (NM_007183) | −95 | NM_007183 | 11187 | ENSG00000184363 | PKP3 |
| chr11 | 47416258 | CpG | −6074 | NR_039707 | 100616222 | ENSG00000264583 | MIR4487 |
| chr11 | 63974672 | exon (NM_031471, exon 2 of 15) | 706 | NM_031471 | 83706 | ENSG00000149781 | FERMT3 |
| chr11 | 165408329 | exon (NM_153253, exon 2 of 16) | 906 | NM_153253 | 6494 | ENSG00000213445 | SIPA1 |
| chr11 | 67204996 | promoter-TSS (NM_005608) | −41 | NM_005608 | 5790 | ENSG00000213402 | PTPRCAP |
| chr11 | 69469526 | TTS (NM_053056) | 13850 | NM_053056 | 595 | ENSG00000110092 | CCND1 |
| chr12 | 7060087 | promoter-TSS (NM_080549) | −56 | NM_080549 | 5777 | ENSG00000111679 | PTPN6 |
| chr12 | 53298578 | promoter-TSS (NM_002273) | 65 | NM_002273 | 3856 | ENSG00000170421 | KRT8 |
| chr12 | 95941769 | promoter-TSS (NM_001278393) | 85 | NM_001278393 | 84101 | ENSG00000136014 | USP44 |
| chr12 | 107038917 | intron (NM_213594, intron 3 of 17) | 39397 | NR_038912 | 100505978 | ENSG00000257711 | LOC100505978 |
| chr12 | 124773568 | 5′ UTR (NM_181709, exon 1 of 3) | 163 | NM_181709 | 144347 | ENSG00000178882 | RFLNA |
| chr12 | 124950569 | exon (NM_001077261, exon 7 of 48) | 101328 | NM_001077261 | 9612 | ENSG00000196498 | NCOR2 |
| chr16 | 1128589 | promoter-TSS (NM_001053) | −46 | NR_027242 | 146336 | ENSG00000261713 | SSTR5-AS1 |
| chr17 | 26732020 | exon (NM_001242366, exon 2 of 5) | 983 | NM_080669 | 113235 | ENSG00000076351 | SLC46A1 |
| chr17 | 41363402 | promoter-TSS (NM_001291586) | −82 | NM_001291588 | 113277 | ENSG00000184988 | TMEM106A |
| chr17 | 43318510 | exon (NM_005892, exon 14 of 27) | −6537 | NR_110326 | 100133991 | ENSG00000267278 | MAP3K14-AS1 |
| chr17 | 79251155 | intron (NM_001037984, intron 6 of 15) | 17802 | NM_001037984 | 124565 | ENSG00000157637 | SLC38A10 |
| chr17 | 80358729 | intron (NM_175902, intron 7 of 9) | −17370 | NM_001369488 | 284004 | ENSG00000169660 | HEXD |
| chr17 | 180847037 | intron (NM_005993, intron 15 of 38) | −49306 | NM_024702 | 79755 | ENSG00000141579 | ZNF750 |
| chr19 | 2085186 | intron (NM_130807, intron 1 of 4) | 10940 | NM_130807 | 126308 | ENSG00000172081 | MOB3A |
| chr19 | 2588141 | CpG | −42278 | NR_107004 | 102465838 | ENSG00000277904 | MIR7850 |
| chr19 | 3179264 | exon (NM_003775, exon 1 of 1) | 829 | NM_003775 | 8698 | ENSG00000125910 | S1PR4 |
| chr19 | 10735906 | promoter-TSS (NM_001363611) | −2 | NM_001363611 | 57153 | ENSG00000129353 | SLC44A2 |
| chr19 | 15568260 | exon (NM_001348028, exon 9 of 11) | 6888 | NM_001348028 | 64926 | ENSG00000105122 | RASAL3 |
| chr19 | 18872937 | CpG | 28961 | NM_000095 | 1311 | ENSG00000105664 | COMP |
| chr19 | 35940325 | intron (NM_005306, intron 2 of 2) | 1350 | NM_001370087 | 2867 | ENSG00000126262 | FFAR2 |
| chr19 | 42703509 | non-coding (NR_073046, exon 5 of 5) | 18098 | NM_001270615 | 162989 | ENSG00000160570 | DEDD2 |
| chr22 | 48027541 | promoter-TSS (NR_033377) | 358 | NR_122046 | 284930 | ENSG00000224271 | LOC284930 |
| chr1 | 2036183 | intron (NM_001033582, intron 1 of 14) | 308 | NM_001033582 | 5590 | ENSG00000067606 | PRKCZ |
| chr1 | 2309984 | non-coding (NR_125361, exon 7 of 7) | 12787 | NM_001301060 | 79906 | ENSG00000116151 | MORN1 |
| chr1 | 2379650 | MLT1C\|LTR\|ERVL-MaLR | −19046 | NM_001303012 | 9651 | ENSG00000149527 | PLCH2 |
| chr1 | 9887711 | Intergenic | −3314 | NM_001009566 | 22883 | ENSG00000171603 | CLSTN1 |
| chr1 | 119870187 | promoter-TSS (NR_146623) | −492 | NR_146623 | 105378934 | ENSG00000232650 | LINC01780 |
| chr1 | 150122937 | 5′ UTR (NM_001304723, exon 1 of 5) | 316 | NM_001304723 | 51177 | ENSG00000023902 | PLEKHO1 |
| chr1 | 155034908 | promoter-TSS (NM_182689) | −947 | NM_182689 | 1945 | ENSG00000243364 | EFNA4 |
| chr1 | 156215318 | exon (NM_024897, exon 3 of 7) | 2274 | NM_001272110 | 79957 | ENSG00000160781 | PAQR6 |
| chr1 | 205631878 | exon (NM_033102, exon 3 of 5) | 17597 | NM_033102 | 85414 | ENSG00000158715 | SLC45A3 |
| chr1 | 234666987 | non-coding (NR_038426, exon 1 of 3) | 207 | NR_038426 | 100506795 | ENSG00000231768 | LINC01354 |
| chr1 | 234908126 | Intergenic | −12567 | NR_148962 | 100130249 | ENSG00000279261 | PP2672 |
| chr1 | 235805303 | CpG | 7497 | NM_001098722 | 2786 | ENSG00000168243 | GNG4 |
| chr1 | 246939116 | Intergenic | −13561 | NR_015422 | 149134 | | LINC01341 |
| chr2 | 3281230 | intron (NM_001330530, intron 3 of 9) | 100159 | NM_001330530 | 7260 | ENSG00000032389 | EIPR1 |
| chr2 | 10567426 | TTS (NM_134421) | 19290 | NR_028374 | 100302743 | ENSG00000206633 | SNORA80B |
| chr2 | 11796686 | CpG | 13419 | NM_012344 | 23620 | ENSG00000169006 | NTSR2 |
| chr2 | 45237589 | Intergenic | −1218 | NM_016932 | 10736 | ENSG00000170577 | SIX2 |
| chr2 | 121432974 | Intergenic | −60258 | NM_001371271 | | | |
| chr2 | 172952783 | 3′ UTR (NM_178120, exon 3 of 3) | 3022 | NM_001038493 | 1745 | ENSG00000144355 | DLX1 |
| chr2 | 240161519 | intron (NM_006037, intron 2 of 26) | −44619 | NR_026664 | 85009 | | MGC16025 |
| chr3 | 134125473 | CpG | −30947 | NR_039951 | 100616281 | ENSG00000284561 | MIR4788 |
| chr3 | 140813669 | CpG | 43762 | NM_080862 | 92369 | ENSG00000175093 | SPSB4 |
| chr3 | 160167217 | promoter-TSS (NM_173084) | −28 | NR_148401 | 100174949 | ENSG00000248710 | TRIM59-1FT80 |
| chr3 | 183891828 | promoter-TSS (NM_001311198) | −640 | NM_001025205 | 1173 | ENSG00000161203 | AP2M1 |
| chr4 | 981385 | 3′ UTR (NM_213613, exon 4 of 4) | 726 | NM_000203 | 3425 | ENSG00000127415 | IDUA |
| chr4 | 1205814 | non-coding (NR_104331, exon 3 of 6) | 2124 | NR_104331 | 285463 | ENSG00000280927 | CTBP1-AS |
| chr4 | 7228168 | intron (NM_020777, intron 1 of 26) | 34090 | NM_020777 | 57537 | ENSG00000184985 | SORCS2 |
| chr5 | 92938049 | Intergenic | 18218 | NR_031754 | 100313887 | ENSG00000284336 | MIR2277 |
| chr5 | 134827036 | CpG | −39437 | NM_001099221 | 497189 | ENSG00000255833 | TIFAB |
| chr5 | 147162061 | promoter-TSS (NM_001282282) | −20 | NM_001270941 | 9832 | ENSG00000176049 | JAKMIP2 |
| chr5 | 179588340 | CpG | 47572 | NM_175062 | 255426 | ENSG00000146090 | RASGEF1C |
| chr6 | 40567217 | CpG | −12345 | NM_020737 | 57497 | ENSG00000156564 | LRFN2 |
| chr6 | 170449317 | Intergenic | −26364 | NR_125878 | 102724511 | | LOC102724511 |
| chr6 | 170578492 | intron (NR_126021, intron 2 of 2) | −2838 | NR_104177 | 401289 | | LINC01624 |
| chr7 | 214477 | MIR3\|SINE\|MIR | 22107 | NM_020223 | 56975 | ENSG00000177706 | FAM20C |
| chr7 | 906626 | intron (NR_160282, intron 20 of 22) | −9428 | NM_015949 | 51608 | ENSG00000239857 | GET4 |
| chr7 | 921951 | intron (NM_015949, intron 1 of 8) | 5952 | NM_015949 | 51608 | ENSG00000239857 | GET4 |
| chr7 | 2968216 | CpG | −84530 | NM_007353 | 2768 | ENSG00000146535 | GNA12 |
| chr7 | 50628618 | promoter-TSS (NM_001242889) | −125 | NM_001242887 | 1644 | ENSG00000132437 | DDC |
| chr7 | 129912387 | intron (NM_001869, intron 4 of 10) | 5942 | NM_001869 | 1358 | ENSG00000158516 | CPA2 |
| chr7 | 151432920 | 5′ UTR (NM_001304527, exon 1 of 14) | 176 | NM_001363698 | 51422 | ENSG00000106617 | PRKAG2 |
| chr8 | 73848837 | exon (NM_004770, exon 3 of 3) | −72073 | NM_003218 | 7013 | ENSG00000147601 | TERF1 |
| chr8 | 93114050 | CpG | 1160 | NM_001198633 | 862 | ENSG00000079102 | RUNX1T1 |
| chr10 | 670687 | intron (NM_014974, intron 1 of 36) | −3607 | NR_147612 | 101930421 | ENSG00000225140 | LOC101930421 |
| chr10 | 123923781 | intron (NM_001291878, intron 1 of 16) | 838 | NM_206860 | 10579 | ENSG00000138162 | TACC2 |
| chr11 | 20625892 | intron (NM_004211, intron 3 of 15) | 5182 | NM_004211 | 9152 | ENSG00000165970 | SLC6A5 |
| chr11 | 61449637 | intron (NM_006133, intron 1 of 19) | 1955 | NM_006133 | 747 | ENSG00000134780 | DAGLA |
| chr11 | 62211890 | CpG | 25588 | NM_003357 | 7356 | ENSG00000149021 | SCGB1A1 |
| chr12 | 104852255 | intron (NM_018413, intron 1 of 2) | 1675 | NM_001173982 | 50515 | ENSG00000171310 | CHST11 |
| chr12 | 110172108 | non-coding (NR_026661, exon 4 of 4) | 20647 | NM_032829 | 84915 | ENSG00000139438 | FAM222A |
| chr12 | 125578255 | CpG | 28469 | NM_023928 | 65985 | ENSG00000081760 | AACS |
| chr14 | 91862713 | intron (NM_001080414, intron 3 of 29) | 21304 | NM_001080414 | 440193 | ENSG00000015133 | CCDC88C |
| chr14 | 95693684 | intron (NM_024734, intron 2 of 12) | −69536 | NM_030621 | 23405 | ENSG00000100697 | DICER1 |
| chr14 | 101157815 | Intergenic | 34478 | NR_024096 | 283601 | ENSG00000196273 | LINC00523 |
| chr15 | 89921572 | CpG | 599 | NR_015411 | 254559 | ENSG00000255571 | MIR9-3HG |
| chr15 | 89959557 | CpG | 16566 | NR_160724 | 105371031 | | LOC105371031 |
| chr15 | 99194331 | promoter-TSS (NR_126453) | −78 | NR_126453 | 104472848 | | IRAIN |
| chr16 | 1195273 | Intergenic | −7596 | NM_021098 | 8912 | ENSG00000196557 | CACNA1H |
| chr16 | 29324459 | intron (NR_002939, intron 2 of 8) | 11078 | NR_002939 | 440352 | | SNX29P2 |
| chr17 | 59480574 | CpG-10965 | 3773 | NM_005994 | 6909 | ENSG00000121068 | TBX2 |
| chr17 | 60827102 | intron (NM_001288779, intron 4 of 11) | 43797 | NR_147886 | 105371855 | ENSG00000265702 | LOC105371855 |
| chr17 | 71744333 | TTS (NR_027146) | 10686 | NM_001278587 | 100134391 | | LOC100134391 |
| chr17 | 72350254 | exon (NM_153209, exon 18 of 20) | 7475 | NM_001080466 | 388419 | ENSG00000204347 | BTBD17 |
| chr17 | 73996145 | promoter-TSS (NR_130467).2 | −653 | NM_001258 | 1018 | ENSG00000250506 | CDK3 |
| chr17 | 76355046 | 5′ UTR (NM_003955, exon 2 of 2) | 943 | NM_003955 | 9021 | ENSG00000184557 | SOCS3 |
| chr17 | 78079381 | exon (NM_000152, exon 3 of 20) | 4258 | NM_001079803 | 2548 | ENSG00000171298 | GAA |
| chr17 | 79223965 | TTS (NM_138570) | 10472 | NR_148426 | 284184 | ENSG00000224877 | NDUFAF8 |
| chr17 | 80019426 | exon (NM_022156, exon 7 of 14) | 4103 | NM_022156 | 64118 | ENSG00000169718 | DUS1L |
| chr17 | 80052807 | intron (NM_004104, intron 3 of 42) | 3056 | NM_004104 | 2194 | ENSG00000169710 | FASN |
| chr17 | 80372675 | intron (NR_033265, intron 2 of 9) | −3379 | NM_173620 | 284004 | ENSG00000169660 | HEXD |
| chr17 | 80832228 | intron (NM_005993, intron 14 of 38) | −34572 | NM_024702 | 79755 | ENSG00000141579 | ZNF750 |
| chr18 | 8659409 | CpG | 47782 | NR_120598 | 100287082 | ENSG00000265962 | GACAT2 |
| chr18 | 13136240 | Intergenic | −82290 | NM_181482 | 753 | ENSG00000168675 | LDLRAD4 |
| chr19 | 2540807 | CpG | 64881 | NM_015675 | 4616 | ENSG00000099860 | GADD45B |
| chr19 | 4566300 | CpG | −6843 | NM_032108 | 10501 | ENSG00000167680 | SEMA6B |
| chr19 | 17790974 | CpG | 7794 | NM_001080421 | 23025 | ENSG00000130477 | UNC13A |
| chr20 | 20432962 | CpG | 84447 | NM_002196 | 3642 | ENSG00000173404 | INSM1 |
| chr20 | 51589516 | 5′ UTR (NM_173485, exon 1 of 3) | 904 | NM_173485 | 128553 | ENSG00000182463 | TSHZ2 |
| chr21 | 38077158 | CpG | 6051 | NM_005069 | 6493 | ENSG00000159263 | SIM2 |
| chr21 | 43441927 | promoter-TSS (NR_119385) | 28 | NR_119385 | 150142 | ENSG00000237232 | ZNF295-AS1 |
| chr1 | 2036220 | promoter-TSS (NM_001033582) | 67 | NM_001033582 | 5590 | ENSG00000067606 | PRKCZ |
| chr1 | 2036226 | promoter-TSS (NM_001033582) | 73 | NM_001033582 | 5590 | ENSG00000067606 | PRKCZ |
| chr1 | 2036232 | promoter-TSS (NM_001033582) | 79 | NM_001033582 | 5590 | ENSG00000067606 | PRKCZ |
| chr1 | 2036252 | promoter-TSS (NM_001033582) | 99 | NM_001033582 | 5590 | ENSG00000067606 | PRKCZ |
| chr1 | 2036395 | intron (NM_001033582, intron 1 of 14) | 242 | NM_001033582 | 5590 | ENSG00000067606 | PRKCZ |
| chr1 | 3697617 | 3′ UTR (NM_020710, exon 7 of 7) | 8268 | NM_001163724 | 388588 | ENSG00000235169 | SMIM1 |
| chr1 | 3697629 | 3′ UTR (NM_020710, exon 7 of 7) | 8281 | NM_001163724 | 388588 | ENSG00000235169 | SMIM1 |
| chr1 | 3697639 | 3′ UTR (NM_020710, exon 7 of 7) | 8290 | NM_001163724 | 388588 | ENSG00000235169 | SMIM1 |
| chr1 | 3697648 | 3′ UTR (NM_020710, exon 7 of 7) | 8299 | NM_001163724 | 388588 | ENSG00000235169 | SMIM1 |
| chr1 | 3697654 | exon (NM_020710, exon 7 of 7) | 8307 | NM_001163724 | 388588 | ENSG00000235169 | SMIM1 |
| chr1 | 3697667 | exon (NM_020710, exon 7 of 7) | 8318 | NM_001163724 | 388588 | ENSG00000235169 | SMIM1 |
| chr1 | 3697692 | exon (NM_020710, exon 7 of 7) | 8343 | NM_001163724 | 388588 | ENSG00000235169 | SMIM1 |
| chr1 | 3697702 | exon (NM_020710, exon 7 of 7) | 8353 | NM_001163724 | 388588 | ENSG00000235169 | SMIM1 |
| chr1 | 6171653 | MET1C\|LTR\|ERVL-MaLR | 65707 | NM_001199863 | 8514 | ENSG00000069424 | KCNAB2 |
| chr1 | 6171833 | exon (NM_015557, exon 36 of 42) | 65887 | NM_001199863 | 8514 | ENSG00000069424 | KCNAB2 |
| chr1 | 6171838 | exon (NM_015557, exon 36 of 42) | 65892 | NM_001199863 | 8514 | ENSG00000069424 | KCNAB2 |
| chr1 | 6171845 | exon (NM_015557, exon 36 of 42) | 65899 | NM_001199863 | 8514 | ENSG00000069424 | KCNAB2 |
| chr1 | 6171867 | exon (NM_015557, exon 36 of 42) | 65922 | NM_001199863 | 8514 | ENSG00000069424 | KCNAB2 |
| chr1 | 9501648 | Intergenic | −12595 | NR_104622 | 100506022 | ENSG00000284693 | LINC02606 |
| chr1 | 10696021 | TTS (NM_001079843) | 160680 | NM_001079843 | 54897 | ENSG00000130940 | CASZ1 |
| chr1 | 10696065 | TTS (NM_001079843) | 160636 | NM_001079843 | 54897 | ENSG00000130940 | CASZ1 |
| chr1 | 18808323 | exon (NM_152375, exon 1 of 1) | 901 | NM_152375 | 127707 | ENSG00000179023 | KLHDC7A |
| chr1 | 18808343 | exon (NM_152375, exon 1 of 11 | 921 | NM_152375 | 127707 | ENSG00000179023 | KLHDC7A |
| chr1 | 18808439 | exon (NM_152375, exon 1 of 1) | 1017 | NM_152375 | 127707 | ENSG00000179023 | KLHDC7A |
| chr1 | 18808469 | exon (NM_152375, exon 1 of 1) | 1047 | NM_152375 | 127707 | ENSG00000179023 | KLHDC7A |
| chr1 | 18808546 | exon (NM_152375, exon 1 of 1) | 1124 | NM_152375 | 127707 | ENSG00000179023 | KLHDC7A |
| chr1 | 18808583 | exon (NM_152375, exon 1 of 1) | 1161 | NM_152375 | 127707 | ENSG00000179023 | KLHDC7A |
| chr1 | 18808621 | exon (NM_152375, exon 1 of 1) | 1199 | NM_152375 | 127707 | ENSG00000179023 | KLHDC7A |
| chr1 | 18808832 | exon (NM_152375, exon 1 of 1) | 1410 | NM_152375 | 127707 | ENSG00000179023 | KLHDC7A |
| chr1 | 18808880 | exon (NM_152375, exon 1 of 1) | 1458 | NM_152375 | 127707 | ENSG00000179023 | KLHDC7A |
| chr1 | 18808929 | exon (NM_152375, exon 1 of 1) | 1509 | NM_152375 | 127707 | ENSG00000179023 | KLHDC7A |
| chr1 | 18808938 | exon (NM_152375, exon 1 of 1) | 1516 | NM_152375 | 127707 | ENSG00000179023 | KLHDC7A |
| chr1 | 18808967 | exon (NM_152375, exon 1 of 1) | 1545 | NM_152375 | 127707 | ENSG00000179023 | KLHDC7A |
| chr1 | 18808991 | exon (NM_152375, exon 1 of 1) | 1569 | NM_152375 | 127707 | ENSG00000179023 | KLHDC7A |
| chr1 | 18809009 | exon (NM_152375, exon 1 of 1) | 1587 | NM_152375 | 127707 | ENSG00000179023 | KLHDC7A |
| chr1 | 18809028 | exon (NM_152375, exon 1 of 1) | 1608 | NM_152375 | 127707 | ENSG00000179023 | KLHDC7A |
| chr1 | 18809041 | exon (NM_152375, exon 1 of 1) | 1619 | NM_152375 | 127707 | ENSG00000179023 | KLHDC7A |
| chr1 | 18809052 | exon (NM_152375, exon 1 of 1) | 1630 | NM_152375 | 127707 | ENSG00000179023 | KLHDC7A |
| chr1 | 18809058 | exon (NM_152375, exon 1 of 1) | 1636 | NM_152375 | 127707 | ENSG00000179023 | KLHDC7A |
| chr1 | 18809064 | exon (NM_152375, exon 1 of 1) | 1642 | NM_152375 | 127707 | ENSG00000179023 | KLHDC7A |
| chr1 | 18809102 | exon (NM_152375, exon 1 of 1) | 1680 | NM_152375 | 127707 | ENSG00000179023 | KLHDC7A |
| chr1 | 18809112 | exon (NM_152375, exon 1 of 1) | 1690 | NM_152375 | 127707 | ENSG00000179023 | KLHDC7A |
| chr1 | 18809126 | exon (NM_152375, exon 1 of 1) | 1704 | NM_152375 | 127707 | ENSG00000179023 | KLHDC7A |
| chr1 | 18809494 | exon (NM_152375, exon 1 of 1) | 2072 | NM_152375 | 127707 | ENSG00000179023 | KLHDC7A |
| chr1 | 20141125 | exon (NM_019062, exon 1 of 1) | 649 | NM_019062 | 54546 | ENSG00000178828 | RNFI86 |
| chr1 | 20141136 | exon (NM_019062, exon 1 of 1) | 638 | NM_019062 | 54546 | ENSG00000178828 | RNF186 |
| chr1 | 20141322 | exon (NM_019062, exon 1 of 1) | 452 | NM_019062 | 54546 | ENSG00000178828 | RNF186 |
| chr1 | 20141344 | exon (NM_019062, exon 1 of 1) | 430 | NM_019062 | 54546 | ENSG00000178828 | RNF186 |
| chr1 | 20141404 | exon (NM_019062, exon 1 of 1) | 370 | NM_019062 | 54546 | ENSG00000178828 | RNF186 |
| chr1 | 20141442 | exon (NM_019062, exon 1 of 1) | 332 | NM_019062 | 54546 | ENSG00000178828 | RNF186 |
| chr1 | 20141462 | exon (NM_019062, exon 1 of 1) | 312 | NM_019062 | 54546 | ENSG00000178828 | RNF186 |
| chr1 | 22195529 | intron (NM_005529, intron 33 of 96) | 56773 | NM_001013693 | 401944 | ENSG00000187942 | LDLRAD2 |
| chr1 | 32252163 | Intergenic | −22224 | NM_001294336 | 576 | ENSG00000121753 | ADGRB2 |
| chr1 | 32252229 | Intergenic | −22290 | NM_001294336 | 576 | ENSG00000121753 | ADGRB2 |
| chr1 | 32252239 | Intergenic | −22300 | NM_001294336 | 576 | ENSG00000121753 | ADGRB2 |
| chr1 | 32252264 | Intergenic | −22325 | NM_001294336 | 576 | ENSG00000121753 | ADGRB2 |
| chr1 | 32252425 | Intergenic | −22486 | NM_001294336 | 576 | ENSG00000121753 | ADGRB2 |
| chr1 | 45103348 | intron (NM_001319957, intron 7 of 13) | 33378 | NR_145816 | 109623472 | | SNORD145 |
| chr1 | 45103390 | intron (NM_001319957, intron 7 of 13) | 33336 | NR_145816 | 109623472 | | SNORD145 |
| chr1 | 45106491 | intron (NM_001319957, intron 7 of 13) | 30235 | NR_145816 | 109623472 | | SNORD145 |
| chr1 | 156215343 | exon (NM_001272106, exon 3 of 6) | 2502 | NM_001272104 | 79957 | ENSG00000160781 | PAQR6 |
| chr1 | 156215371 | exon (NM_001272106, exon 3 of 6) | 2474 | NM_001272104 | 79957 | ENSG00000160781 | PAQR6 |
| chr1 | 156215401 | exon (NM_001272106, exon 3 of 6) | 2444 | NM_001272104 | 79957 | ENSG00000160781 | PAQR6 |
| chr1 | 156215416 | exon (NM_001272106, exon 3 of 6) | 2429 | NM_001272104 | 79957 | ENSG00000160781 | PAQR6 |
| chr1 | 156215430 | exon (NM_001272106, exon 3 of 6) | 2415 | NM_001272104 | 79957 | ENSG00000160781 | PAQR6 |
| chr1 | 156215445 | exon (NM_024897, exon 4 of 7) | 2400 | NM_001272104 | 79957 | ENSG00000160781 | PAQR6 |
| chr1 | 156215454 | CpG | 2391 | NM_001272104 | 79957 | ENSG00000160781 | PAQR6 |
| chr1 | 156215491 | CpG | 2353 | NM_001272104 | 79957 | ENSG00000160781 | PAQR6 |
| chr1 | 156215506 | CpG | 2338 | NM_001272104 | 79957 | ENSG00000160781 | PAQR6 |
| chr1 | 156215520 | CpG | 2325 | NM_001272104 | 79957 | ENSG00000160781 | PAQR6 |
| chr1 | 156215525 | CpG | 2320 | NM_001272104 | 79957 | ENSG00000160781 | PAQR6 |
| chr1 | 156215530 | CpG | 2315 | NM_001272104 | 79957 | ENSG00000160781 | PAQR6 |
| chr1 | 156215563 | CpG | 2282 | NM_001272104 | 79957 | ENSG00000160781 | PAQR6 |
| chr1 | 156215590 | exon (NM_024897, exon 3 of 7) | 2253 | NM_001272104 | 79957 | ENSG00000160781 | PAQR6 |
| chr1 | 156215601 | exon (NM_024897, exon 3 of 7) | 2244 | NM_001272104 | 79957 | ENSG00000160781 | PAQR6 |
| chr1 | 156215708 | non-coding (NR_073610, exon 3 of 7) | 2137 | NM_001272104 | 79957 | ENSG00000160781 | PAQR6 |
| chr1 | 156215720 | non-coding (NR_073610, exon 3 of 7) | 2125 | NM_001272104 | 79957 | ENSG00000160781 | PAQR6 |
| chr1 | 156215726 | non-coding (NR_073610, exon 3 of 7) | 2117 | NM_001272104 | 79957 | ENSG00000160781 | PAQR6 |
| chr1 | 156215737 | non-coding (NR_073610, exon 3 of 7) | 2108 | NM_001272104 | 79957 | ENSG00000160781 | PAQR6 |
| chr1 | 203259747 | Intergenic | 2923 | NR_117098 | 100506775 | ENSG00000231507 | LINC01353 |
| chr1 | 203259757 | Intergenic | 2931 | NR_117098 | 100506775 | ENSG00000231507 | LINC01353 |
| chr1 | 203259770 | Intergenic | 2944 | NR_117098 | 100506775 | ENSG00000231507 | LINC01353 |
| chr1 | 203259836 | Intergenic | 3010 | NR_117098 | 100506775 | ENSG00000231507 | LINC01353 |
| chr1 | 203259850 | Intergenic | 3024 | NR_117098 | 100506775 | ENSG00000231507 | LINC01353 |
| chr1 | 203259921 | Intergenic | 3095 | NR_117098 | 100506775 | ENSG00000231507 | LINC01353 |
| chr1 | 236311219 | intron (NM_003272, intron 1 of 6) | 5382 | NM_003272 | 7107 | ENSG00000077585 | GPR137B |
| chr1 | 236311342 | intron (NM_003272, intron 1 of 6) | 5505 | NM_003272 | 7107 | ENSG00000077585 | GPR137B |
| chr1 | 237129554 | AluJb\|SINE\|Alu | 38185 | NM_001276687 | 645745 | ENSG00000244020 | MT1HL1 |
| chr1 | 237134163 | AluSx1\|SINE\|Alu | 33576 | NM_001276687 | 645745 | ENSG00000244020 | MT1HL1 |
| chr1 | 237167480 | TTS (NM_001276687) | 259 | NM_001276687 | 645745 | ENSG00000244020 | MT1HL1 |
| chr1 | 237194084 | AluSx1\|SINE\|Alu | −11398 | NM_001035 | 6262 | ENSG00000198626 | RYR2 |
| chr1 | 237194093 | AluSx1\|SINE\|Alu | −11389 | NM_001035 | 6262 | ENSG00000198626 | RYR2 |
| chr1 | 237194098 | AluSx1\|SINE\|Alu | −11384 | NM_001035 | 6262 | ENSG00000198626 | RYR2 |
| chr1 | 237195314 | LTR78\|LTR\|ERV1 | −10168 | NM_001035 | 6262 | ENSG00000198626 | RYR2 |
| chr1 | 237195411 | LTR78\|LTR\|ERV1 | −10071 | NM_001035 | 6262 | ENSG00000198626 | RYR2 |
| chr1 | 237212336 | intron (NM_001035, intron 1 of 104) | 6854 | NM_001035 | 6262 | ENSG00000198626 | RYR2 |
| chr1 | 237220609 | AluSc8\|SINE\|Alu | 15127 | NM_001035 | 6262 | ENSG00000198626 | RYR2 |
| chr1 | 237220641 | AluSc8\|SINE\|Alu | 15161 | NM_001035 | 6262 | ENSG00000198626 | RYR2 |
| chr1 | 237222098 | GC_rich\|Low_complexity\|Low_complexity | 16616 | NM_001035 | 6262 | ENSG00000198626 | RYR2 |
| chr1 | 237222106 | GC_rich\|Low_complexity\|Low_complexity | 16624 | NM_001035 | 6262 | ENSG00000198626 | RYR2 |
| chr1 | 237235121 | MER52A\|LTR\|ERV1 | 29639 | NM_001035 | 6262 | ENSG00000198626 | RYR2 |
| chr1 | 237235204 | MER52A\|LTR\|ERV1 | 29722 | NM_001035 | 6262 | ENSG00000198626 | RYR2 |
| chr1 | 237235522 | MER52A\|LTR\|ERV1 | 30040 | NM_001035 | 6262 | ENSG00000198626 | RYR2 |
| chr1 | 237235567 | MER52A\|LTR\|ERV1 | 30086 | NM_001035 | 6262 | ENSG00000198626 | RYR2 |
| chr1 | 237235607 | FLAM_C\|SINE\|Alu | 30125 | NM_001035 | 6262 | ENSG00000198626 | RYR2 |
| chr1 | 237256273 | intron (NM_001035, intron 1 of 104) | 50791 | NM_001035 | 6262 | ENSG00000198626 | RYR2 |
| chr1 | 237256316 | MER3\|DNA\|hAT-Charlie | 50834 | NM_001035 | 6262 | ENSG00000198626 | RYR2 |
| chr1 | 237265786 | intron (NM_001035, intron 1 of 104) | 60304 | NM_001035 | 6262 | ENSG00000198626 | RYR2 |
| chr1 | 237265877 | AluSz\|SINE\|Alu | 60395 | NM_001035 | 6262 | ENSG00000198626 | RYR2 |
| chr1 | 237267786 | intron (NM_001035, intron 1 of 104) | 62304 | NM_001035 | 6262 | ENSG00000198626 | RYR2 |
| chr1 | 237267812 | intron (NM_001035, intron 1 of 104) | 62330 | NM_001035 | 6262 | ENSG00000198626 | RYR2 |
| chr1 | 237267886 | intron (NM_001035, intron 1 of 104) | 62404 | NM_001035 | 6262 | ENSG00000198626 | RYR2 |
| chr1 | 237306208 | intron (NM_001035, intron 1 of 104) | 100726 | NM_001035 | 6262 | ENSG00000198626 | RYR2 |
| chr1 | 237306260 | intron (NM_001035, intron 1 of 104) | 100778 | NM_001035 | 6262 | ENSG00000198626 | RYR2 |
| chr1 | 237343254 | intron (NM_001035, intron 1 of 104) | 137772 | NM_001035 | 6262 | ENSG00000198626 | RYR2 |
| chr1 | 237359169 | MIR\|SINE\|MIR | 153687 | NM_001035 | 6262 | ENSG00000198626 | RYR2 |
| chr1 | 237383690 | MLT2B3\|LTR\|ERVL | 178208 | NM_001035 | 6262 | ENSG00000198626 | RYR2 |
| chr1 | 237392760 | intron (NM_001035, intron 1 of 104) | 187278 | NM_001035 | 6262 | ENSG00000198626 | RYR2 |
| chr1 | 237408076 | LTR9\|LTR\|ERV1 | 202594 | NM_001035 | 6262 | ENSG00000198626 | RYR2 |
| chr1 | 237408091 | LTR9\|LTR\|ERV1 | 202609 | NM_001035 | 6262 | ENSG00000198626 | RYR2 |
| chr1 | 237473057 | LTR12C\|LTR\|ERV1 | −161360 | NR_039626 | 100616141 | ENSG00000266262 | MIR4428 |
| chr1 | 237473071 | LTR12C\|LTR\|ERV1 | −161346 | NR_039626 | 100616141 | ENSG00000266262 | MIR4428 |
| chr1 | 237473091 | LTR12C\|LTR\|ERV1 | −161326 | NR_039626 | 100616141 | ENSG00000266262 | MIR4428 |
| chr1 | 237473164 | LTR12C\|LTR\|ERV1 | −161253 | NR_039626 | 100616141 | ENSG00000266262 | MIR4428 |
| chr1 | 237538162 | intron (NM_001035, intron 7 of 104) | −96255 | NR_039626 | 100616141 | ENSG00000266262 | MIR4428 |
| chr1 | 237584276 | AluSc8\|SINE\|Alu | −50141 | NR_039626 | 100616141 | ENSG00000266262 | MIR4428 |
| chr1 | 237661252 | AluSz6\|SINE\|Alu | 26835 | NR_039626 | 100616141 | ENSG00000266262 | MIR4428 |
| chr1 | 237661262 | AluSz6\|SINE\|Alu | 26845 | NR_039626 | 100616141 | ENSG00000266262 | MIR4428 |
| chr1 | 237661322 | AluSz6\|SINE\|Alu | 26905 | NR_039626 | 100616141 | ENSG00000266262 | MIR4428 |
| chr1 | 237693526 | AluY\|SINE\|Alu | 59109 | NR_039626 | 100616141 | ENSG00000266262 | MIR4428 |
| chr1 | 237693534 | AluY\|SINE\|Alu | 59117 | NR_039626 | 100616141 | ENSG00000266262 | MIR4428 |
| chr1 | 237699930 | AluY\|SINE\|Alu | 65513 | NR_039626 | 100616141 | ENSG00000266262 | MIR4428 |
| chr1 | 237699938 | AluY\|SINE\|Alu | 65521 | NR_039626 | 100616141 | ENSG00000266262 | MIR4428 |
| chr1 | 237699988 | AluY\|SINE\|Alu | 65571 | NR_039626 | 100616141 | ENSG00000266262 | MIR4428 |
| chr1 | 237809365 | AluY\|SINE\|Alu | 174948 | NR_039626 | 100616141 | ENSG00000266262 | MIR4428 |
| chr1 | 237815260 | AluY\|SINE\|Alu | 180843 | NR_039626 | 100616141 | ENSG00000266262 | MIR4428 |
| chr1 | 241402493 | AluSc8\|SINE\|Alu | 106923 | NR_036069 | 100422856 | ENSG00000265831 | MIR3123 |
| chr1 | 241402516 | AluSc8\|SINE\|Alu | 106946 | NR_036069 | 100422856 | ENSG00000265831 | MIR3123 |
| chr2 | 9248023 | Intergenic | −98869 | NM_001135191 | 8853 | ENSG00000151693 | ASAP2 |
| chr2 | 9248368 | Intergenic | −98524 | NM_001135191 | 8853 | ENSG00000151693 | ASAP2 |
| chr2 | 9248435 | Intergenic | −98457 | NM_001135191 | 8853 | ENSG00000151693 | ASAP2 |
| chr2 | 9248469 | Intergenic | −98423 | NM_001135191 | 8853 | ENSG00000151693 | ASAP2 |
| chr2 | 25299679 | intron (NM_001319099, intron 1 of 22) | 34349 | NM_001319099 | 22979 | ENSG00000084710 | EFR3B |
| chr2 | 25299815 | intron (NM_001319099, intron 1 of 22) | 34485 | NM_001319099 | 22979 | ENSG00000084710 | EFR3B |
| chr2 | 25299928 | intron (NM_001319099, intron 1 of 22) | 34598 | NM_001319099 | 22979 | ENSG00000084710 | EFR3B |
| chr2 | 43102420 | Intergenic | −82699 | NM_012205 | 23498 | ENSG00000162882 | HAAO |
| chr2 | 43107259 | Intergenic | −87538 | NM_012205 | 23498 | ENSG00000162882 | HAAO |
| chr2 | 43107400 | Intergenic | −87679 | NM_012205 | 23498 | ENSG00000162882 | HAAO |
| chr2 | 43107414 | Intergenic | −87693 | NM_012205 | 23498 | ENSG00000162882 | HAAO |
| chr2 | 43107438 | Intergenic | −87717 | NM_012205 | 23498 | ENSG00000162882 | HAAO |
| chr2 | 43107456 | Intergenic | −87735 | NM_012205 | 23498 | ENSG00000162882 | HAAO |
| chr2 | 43107465 | Intergenic | −87744 | NM_012205 | 23498 | ENSG00000162882 | HAAO |
| chr2 | 43107514 | Intergenic | −87793 | NM_012205 | 23498 | ENSG00000162882 | HAAO |
| chr2 | 43107532 | Intergenic | −87811 | NM_012205 | 23498 | ENSG00000162882 | HAAO |
| chr2 | 43107650 | Intergenic | −87929 | NM_012205 | 23498 | ENSG00000162882 | HAAO |
| chr2 | 45235684 | CpG | 905 | NM_016932 | 10736 | ENSG00000170577 | SIX2 |
| chr2 | 45235702 | exon (NM_016932, exon 1 of 2) | 887 | NM_016932 | 10736 | ENSG00000170577 | SEX2 |
| chr2 | 45235710 | exon (NM_016932, exon 1 of 2) | 879 | NM_016932 | 10736 | ENSG00000170577 | SEX2 |
| chr2 | 207972376 | intron (NR_073108, intron 3 of 4) | 2419 | NR_036227 | 100423036 | ENSG00000253008 | MIR2355 |
| chr2 | 207972408 | intron (NR_073108, intron 3 of 4) | 2387 | NR_036227 | 100423036 | ENSG00000253008 | MIR2355 |
| chr2 | 220509654 | Intergenic | 17304 | NM_005070 | 6508 | ENSG00000114923 | SLC4A3 |
| chr2 | 220509693 | Intergenic | 17343 | NM_005070 | 6508 | ENSG00000114923 | SLC4A3 |
| chr2 | 220509705 | Intergenic | 17355 | NM_005070 | 6508 | ENSG00000114923 | SLC4A3 |
| chr2 | 220509776 | Intergenic | 17426 | NM_005070 | 6508 | ENSG00000114923 | SLC4A3 |
| chr2 | 220509830 | Intergenic | 17480 | NM_005070 | 6508 | ENSG00000114923 | SLC4A3 |
| chr2 | 241233035 | Intergenic | −142043 | NM_002081 | 2817 | ENSG00000063660 | GPC1 |
| chr2 | 241233048 | Intergenic | −142030 | NM_002081 | 2817 | ENSG00000063660 | GPC1 |
| chr2 | 241233055 | Intergenic | −142023 | NM_002081 | 2817 | ENSG00000063660 | GPC1 |
| chr2 | 241233084 | Intergenic | −141994 | NM_002081 | 2817 | ENSG00000063660 | GPC1 |
| chr2 | 241233090 | Intergenic | −141988 | NM_002081 | 2817 | ENSG00000063660 | GPC1 |
| chr2 | 241233169 | Intergenic | −141909 | NM_002081 | 2817 | ENSG00000063660 | GPC1 |
| chr2 | 241233214 | Intergenic | −141864 | NM_002081 | 2817 | ENSG00000063660 | GPC1 |
| chr3 | 111431516 | intron (NM_001185106, intron 2 of 4) | −19825 | NM_001134437 | 90102 | ENSG00000144824 | PHLDB2 |
| chr3 | 111431559 | intron (NM_001185106, intron 2 of 4) | −19782 | NM_001134437 | 90102 | ENSG00000144824 | PHLDB2 |
| chr4 | 3309964 | Intergenic | −5908 | NM_198229 | 6002 | ENSG00000159788 | RGS12 |
| chr4 | 3310004 | CpG | −5868 | NM_198229 | 6002 | ENSG00000159788 | RGS12 |
| chr4 | 3311787 | Intergenic | −4085 | NM_198229 | 6002 | ENSG00000159788 | RGS12 |
| chr4 | 3311917 | Intergenic | −3955 | NM_198229 | 6002 | ENSG00000159788 | RGS12 |
| chr4 | 7228254 | intron (NM_020777, intron 1 of 26) | 33991 | NM_020777 | 57537 | ENSG00000184985 | SORCS2 |
| chr4 | 7228263 | intron (NM_020777, intron 1 of 26) | 34001 | NM_020777 | 57537 | ENSG00000184985 | SORCS2 |
| chr4 | 7228304 | intron (NM_020777, intron 1 of 26) | 34041 | NM_020777 | 57537 | ENSG00000184985 | SORCS2 |
| chr4 | 7228329 | intron (NM_020777, intron 1 of 26) | 34066 | NM_020777 | 57537 | ENSG00000184985 | SORCS2 |
| chr4 | 7326173 | intron (NM_020777, intron 1 of 26) | 13999 | NR_039961 | 100616471 | ENSG00000264658 | MIR4798 |
| chr4 | 7326188 | intron (NM_020777, intron 1 of 26) | 14013 | NR_039961 | 100616471 | ENSG00000264658 | MIR4798 |
| chr4 | 7326270 | intron (NM_020777, intron 1 of 26) | 14095 | NR_039961 | 100616471 | ENSG00000264658 | MIR4798 |
| chr4 | 7326284 | intron (NM_020777, intron 1 of 26) | 14109 | NR_039961 | 100616471 | ENSG00000264658 | MIR4798 |
| chr4 | 155758143 | HERVK22-int\|LTR\|ERVK | 55488 | NM_001277173 | 166863 | ENSG00000151962 | RBM46 |
| chr4 | 155763679 | AluY\|SINE\|Alu | 61024 | NM_001277173 | 166863 | ENSG00000151962 | RBM46 |
| chr4 | 155763688 | AluY\|SINE\|Alu | 61033 | NM_001277173 | 166863 | ENSG00000151962 | RBM46 |
| chr4 | 155763695 | AluY\|SINE\|Alu | 61040 | NM_001277173 | 166863 | ENSG00000151962 | RBM46 |
| chr4 | 155812513 | AluJb\|SINE\|Alu | 109858 | NM_001277173 | 166863 | ENSG00000151962 | RBM46 |
| chr4 | 155912924 | Intergenic | 210269 | NM_001277173 | 166863 | ENSG00000151962 | RBM46 |
| chr4 | 155965520 | LTR12C\|LTR\|ERV1 | −164266 | NM_000910 | 4887 | ENSG00000185149 | NPY2R |
| chr4 | 155965532 | LTR12C\|LTR\|ERV1 | −164254 | NM_000910 | 4887 | ENSG00000185149 | NPY2R |
| chr4 | 155965538 | LTR12C\|LTR\|ERV1 | −164248 | NM_000910 | 4887 | ENSG00000185149 | NPY2R |
| chr4 | 155965544 | LTR12C\|LTR\|ERV1 | −164242 | NM_000910 | 4887 | ENSG00000185149 | NPY2R |
| chr4 | 155965553 | LTR12C\|LTR\|ERV1 | −164233 | NM_000910 | 4887 | ENSG00000185149 | NPY2R |
| chr4 | 155992200 | AluSq\|SINE\|Alu | −137586 | NM_000910 | 4887 | ENSG00000185149 | NPY2R |
| chr4 | 156002533 | Intergenic | −127253 | NM_000910 | 4887 | ENSG00000185149 | NPY2R |
| chr5 | 1003895 | Intergenic | −5020 | NM_033120 | 85409 | ENSG00000145506 | NKD2 |
| chr5 | 1004045 | CpG | −4870 | NM_033120 | 85409 | ENSG00000145506 | NKD2 |
| chr5 | 1004051 | CpG | −4864 | NM_033120 | 85409 | ENSG00000145506 | NKD2 |
| chr5 | 1004064 | CpG | −4851 | NM_033120 | 85409 | ENSG00000145506 | NKD2 |
| chr5 | 1004077 | CpG | −4838 | NM_033120 | 85409 | ENSG00000145506 | NKD2 |
| chr5 | 1004089 | CpG | −4825 | NM_033120 | 85409 | ENSG00000145506 | NKD2 |
| chr5 | 1004118 | CpG | −4797 | NM_033120 | 85409 | ENSG00000145506 | NKD2 |
| chr5 | 1004128 | CpG | −4787 | NM_033120 | 85409 | ENSG00000145506 | NKD2 |
| chr5 | 1004135 | CpG | −4779 | NM_033120 | 85409 | ENSG00000145506 | NKD2 |
| chr5 | 1004148 | CpG | −4767 | NM_033120 | 85409 | ENSG00000145506 | NKD2 |
| chr5 | 1004157 | CpG | −4758 | NM_033120 | 85409 | ENSG00000145506 | NKD2 |
| chr5 | 1004184 | CpG | −4731 | NM_033120 | 85409 | ENSG00000145506 | NKD2 |
| chr5 | 1004198 | CpG | −4717 | NM_033120 | 85409 | ENSG00000145506 | NKD2 |
| chr5 | 1004234 | CpG | −4681 | NM_033120 | 85409 | ENSG00000145506 | NKD2 |
| chr5 | 1004243 | CpG | −4668 | NM_033120 | 85409 | ENSG00000145506 | NKD2 |
| chr5 | 1004259 | CpG | −4656 | NM_033120 | 85409 | ENSG00000145506 | NKD2 |
| chr5 | 1004269 | CpG | −4646 | NM_033120 | 85409 | ENSG00000145506 | NKD2 |
| chr5 | 1004288 | CpG | −4627 | NM_033120 | 85409 | ENSG00000145506 | NKD2 |
| chr5 | 1004304 | CpG | −4611 | NM_033120 | 85409 | ENSG00000145506 | NKD2 |
| chr5 | 1004351 | CpG | −4564 | NM_033120 | 85409 | ENSG00000145506 | NKD2 |
| chr5 | 1004357 | CpG | −4557 | NM_033120 | 85409 | ENSG00000145506 | NKD2 |
| chr5 | 1004365 | CpG | −4550 | NM_033120 | 85409 | ENSG00000145506 | NKD2 |
| chr5 | 134827336 | CpG | −39302 | NM_001099221 | 497189 | ENSG00000255833 | TIFAB |
| chr5 | 134827353 | CpG | −39314 | NM_001099221 | 497189 | ENSG00000255833 | TIFAB |
| chr5 | 134827364 | CpG | −39325 | NM_001099221 | 497189 | ENSG00000255833 | TIFAB |
| chr5 | 134827378 | CpG | −39339 | NM_001099221 | 497189 | ENSG00000255833 | TIFAB |
| chr5 | 138441347 | intron (NM_001037633, intron 4 of 10) | 92707 | NM_001037633 | 64374 | ENSG00000120725 | SIL1 |
| chr5 | 138441377 | intron (NM_001037633, intron 4 of 10) | 92677 | NM_001037633 | 64374 | ENSG00000120725 | SIL1 |
| chr5 | 138441561 | intron (NM_001037633, intron 4 of 10) | 92494 | NM_001037633 | 64374 | ENSG00000120725 | SIL1 |
| chr5 | 179401802 | intron (NM_018434, intron 5 of 8) | 40593 | NR_029885 | 442908 | ENSG00000198995 | MIR340 |
| chr5 | 179401833 | intron (NM_018434, intron 5 of 8) | 40562 | NR_029885 | 442908 | ENSG00000198995 | MIR340 |
| chr5 | 179401843 | intron (NM_018434, intron 5 of 8) | 40552 | NR_029885 | 442908 | ENSG00000198995 | MIR340 |
| chr5 | 179401853 | intron (NM_018434, intron 5 of 8) | 40542 | NR_029885 | 442908 | ENSG00000198995 | MIR340 |
| chr5 | 179401872 | intron (NM_018434, intron 5 of 8) | 40523 | NR_029885 | 442908 | ENSG00000198995 | MIR340 |
| chr5 | 179588495 | CpG | 47714 | NM_175062 | 255426 | ENSG00000146090 | RASGEF1C |
| chr5 | 179588504 | CpG | 47705 | NM_175062 | 255426 | ENSG00000146090 | RASGEF1C |
| chr5 | 179588512 | CpG | 47697 | NM_175062 | 255426 | ENSG00000146090 | RASGEF1C |
| chr5 | 179588528 | CpG | 47680 | NM_175062 | 255426 | ENSG00000146090 | RASGEF1C |
| chr5 | 179588550 | CpG | 47659 | NM_175062 | 255426 | ENSG00000146090 | RASGEF1C |
| chr5 | 179588590 | CpG | 47619 | NM_175062 | 255426 | ENSG00000146090 | RASGEF1C |
| chr5 | 179588604 | CpG | 47604 | NM_175062 | 255426 | ENSG00000146090 | RASGEF1C |
| chr5 | 179588617 | CpG | 47592 | NM_175062 | 255426 | ENSG00000146090 | RASGEF1C |
| chr5 | 179588625 | CpG | 47584 | NM_175062 | 255426 | ENSG00000146090 | RASGEF1C |
| chr5 | 179588631 | CpG | 47578 | NM_175062 | 255426 | ENSG00000146090 | RASGEF1C |
| chr5 | 179588672 | CpG | 47537 | NM_175062 | 255426 | ENSG00000146090 | RASGEF1C |
| chr5 | 179588687 | CpG | 47522 | NM_175062 | 255426 | ENSG00000146090 | RASGEF1C |
| chr5 | 179588704 | CpG | 47504 | NM_175062 | 255426 | ENSG00000146090 | RASGEF1C |
| chr5 | 179588719 | CpG | 47490 | NM_175062 | 255426 | ENSG00000146090 | RASGEF1C |
| chr5 | 179597660 | intron (NM_175062, intron 1 of 13) | 38549 | NM_175062 | 255426 | ENSG00000146090 | RASGEF1C |
| chr5 | 179597771 | intron (NM_175062, intron 1 of 13) | 38438 | NM_175062 | 255426 | ENSG00000146090 | RASGEF1C |
| chr5 | 179597812 | intron (NM_175062, intron 1 of 13) | 38397 | NM_175062 | 255426 | ENSG00000146090 | RASGEF1C |
| chr6 | 64151793 | LTR12C\|LTR\|ERV1 | −121907 | NM_016571 | 51557 | ENSG00000146166 | LGSN |
| chr6 | 157549787 | Intergenic | 192228 | NM_001363725 | 57492 | ENSG00000049618 | ARID1B |
| chr6 | 157549797 | Intergenic | 192238 | NM_001363725 | 57492 | ENSG00000049618 | ARID1B |
| chr6 | 157549848 | Intergenic | 192289 | NM_001363725 | 57492 | ENSG00000049618 | ARID1B |
| chr6 | 157550494 | Intergenic | 192935 | NM_001363725 | 57492 | ENSG00000049618 | ARID1B |
| chr6 | 157550509 | Intergenic | 192950 | NM_001363725 | 57492 | ENSG00000049618 | ARID1B |
| chr7 | 1447696 | CpG | 51408 | NM_182924 | 79778 | ENSG00000164877 | MICALL2 |
| chr7 | 1447734 | CpG | 51370 | NM_182924 | 79778 | ENSG00000164877 | MICALL2 |
| chr7 | 1447746 | CpG | 51358 | NM_182924 | 79778 | ENSG00000164877 | MICALL2 |
| chr7 | 1447760 | CpG | 51344 | NM_182924 | 79778 | ENSG00000164877 | MICALL2 |
| chr7 | 1447774 | CpG | 51329 | NM_182924 | 79778 | ENSG00000164877 | MICALL2 |
| chr7 | 1447782 | CpG | 51322 | NM_182924 | 79778 | ENSG00000164877 | MICALL2 |
| chr7 | 2006576 | L1MEf\|LINE\|L1 | −26371 | NM_001304525 | 8379 | ENSG00000002822 | MAD1L1 |
| chr7 | 2006674 | L1MEf\|LINE\|L1 | −26469 | NM_001304525 | 8379 | ENSG00000002822 | MAD1L1 |
| chr7 | 2007262 | L1MC4a\|LINE\|L1 | −27057 | NM_001304525 | 8379 | ENSG00000002822 | MAD1L1 |
| chr7 | 2007279 | L1MC4a\|LINE\|L1 | −27074 | NM_001304525 | 8379 | ENSG00000002822 | MAD1L1 |
| chr7 | 2007309 | L1MC4a\|LINE\|L1 | −27104 | NM_001304525 | 8379 | ENSG00000002822 | MAD1L1 |
| chr7 | 2007394 | intron (NM_001304523, intron 14 of 17) | −27189 | NM_001304525 | 8379 | ENSG00000002822 | MAD1L1 |
| chr7 | 2968280 | exon (NM_032415, exon 13 of 25) | −84340 | NM_007353 | 2768 | ENSG00000146535 | GNA12 |
| chr7 | 2968288 | exon (NM_032415, exon 13 of 25) | −84350 | NM_007353 | 2768 | ENSG00000146535 | GNA12 |
| chr7 | 2968314 | exon (NM_032415, exon 13 of 25) | −84374 | NM_007353 | 2768 | ENSG00000146535 | GNA12 |
| chr7 | 2968320 | exon (NM_032415, exon 13 of 25) | −84380 | NM_007353 | 2768 | ENSG00000146535 | GNA12 |
| chr7 | 2968352 | CpG | −84412 | NM_007353 | 2768 | ENSG00000146535 | GNA12 |
| chr7 | 2968357 | CpG | −84417 | NM_007353 | 2768 | ENSG00000146535 | GNA12 |
| chr7 | 2968405 | CpG | −84465 | NM_007353 | 2768 | ENSG00000146535 | GNA12 |
| chr7 | 2968411 | CpG | −84471 | NM_007353 | 2768 | ENSG00000146535 | GNA12 |
| chr7 | 2968426 | CpG | −84486 | NM_007353 | 2768 | ENSG00000146535 | GNA12 |
| chr7 | 2968440 | CpG | −84502 | NM_007353 | 2768 | ENSG00000146535 | GNA12 |
| chr7 | 2968454 | CpG | −84516 | NM_007353 | 2768 | ENSG00000146535 | GNA12 |
| chr7 | 2968489 | CpG | −84553 | NM_007353 | 2768 | ENSG00000146535 | GNA12 |
| chr7 | 2968504 | CpG | −84565 | NM_007353 | 2768 | ENSG00000146535 | GNA12 |
| chr7 | 2968528 | CpG | −84588 | NM_007353 | 2768 | ENSG00000146535 | GNA12 |
| chr7 | 2968552 | CpG | −84612 | NM_007353 | 2768 | ENSG00000146535 | GNA12 |
| chr7 | 2968558 | CpG | −84618 | NM_007353 | 2768 | ENSG00000146535 | GNA12 |
| chr7 | 2968565 | CpG | −84626 | NM_007353 | 2768 | ENSG00000146535 | GNA12 |
| chr7 | 2968585 | CpG | −84645 | NM_007353 | 2768 | ENSG00000146535 | GNA12 |
| chr7 | 2968593 | CpG | −84653 | NM_007353 | 2768 | ENSG00000146535 | GNA12 |
| chr7 | 2968625 | intron (NM_001324281, intron 13 of 25) | −84685 | NM_007353 | 2768 | ENSG00000146535 | GNA12 |
| chr7 | 2968684 | intron (NM_001324281, intron 13 of 25) | −84744 | NM_007353 | 2768 | ENSG00000146535 | GNA12 |
| chr7 | 2968706 | intron (NM_001324281, intron 13 of 25) | −84766 | NM_007353 | 2768 | ENSG00000146535 | GNA12 |
| chr7 | 26667584 | Intergenic | −9904 | NR_161202 | 285941 | | C7orf71 |
| chr7 | 26667613 | Intergenic | −9875 | NR_161202 | 285941 | | C7orf71 |
| chr7 | 26667674 | Intergenic | −9814 | NR_161202 | 285941 | | C7orf71 |
| chr7 | 26667709 | Intergenic | −9779 | NR_161202 | 285941 | | C7orf71 |
| chr7 | 35505816 | Intergenic | 152352 | NR_038864 | 401324 | | LOC401324 |
| chr7 | 35505825 | Intergenic | 152361 | NR_038864 | 401324 | | LOC401324 |
| chr7 | 41086116 | Intergenic | −55084 | NR_110832 | 101928773 | ENSG00000224017 | LINC01449 |
| chr7 | 41086123 | Intergenic | −55077 | NR_110832 | 101928773 | ENSG00000224017 | LINC01449 |
| chr7 | 41086140 | Intergenic | −55060 | NR_110832 | 101928773 | ENSG00000224017 | LINC01449 |
| chr7 | 101622017 | intron (NM_001202544, intron 2 of 21) | 161113 | NM_181552 | 1523 | ENSG00000257923 | CUX1 |
| chr7 | 101622057 | intron (NM_001202544, intron 2 of 21) | 161153 | NM_181552 | 1523 | ENSG00000257923 | CUX1 |
| chr7 | 101622176 | L2a\|LINE\|L2 | 161275 | NM_181552 | 1523 | ENSG00000257923 | CUX1 |
| chr7 | 115992730 | Intergenic | −65951 | NR_130922 | 102724434 | ENSG00000237870 | LOC102724434 |
| chr7 | 115992814 | L2\|LINE\|L2 | −66035 | NR_130922 | 102724434 | ENSG00000237870 | LOC102724434 |
| chr7 | 115998982 | AluY\|SINE\|Alu | −72203 | NR_130922 | 102724434 | ENSG00000237870 | LOC102724434 |
| chr7 | 115999026 | AluY\|SINE\|Alu | −72247 | NR_130922 | 102724434 | ENSG00000237870 | LOC102724434 |
| chr7 | 115999448 | AluSx1\|SINE\|Alu | −72669 | NR_130922 | 102724434 | ENSG00000237870 | LOC102724434 |
| chr7 | 115999464 | AluSx1\|SINE\|Alu | −72685 | NR_130922 | 102724434 | ENSG00000237870 | LOC102724434 |
| chr7 | 116099337 | HERVH-int\|LTR\|ERV1 | −40453 | NM_001233 | 858 | ENSG00000105971 | CAV2 |
| chr7 | 150668588 | intron (NM_000238, intron 2 of 14) | 6812 | NM_172056 | 3757 | ENSG00000055118 | KCNH2 |
| chr7 | 150669120 | intron (NM_000238, intron 2 of 14) | 6280 | NM_172056 | 3757 | ENSG00000055118 | KCNH2 |
| chr7 | 150811991 | exon (NM_001308305, exon 1 of 9) | 268 | NM_001308305 | 116988 | ENSG00000133612 | AGAP3 |
| chr7 | 150812075 | exon (NM_001308305, exon 1 of 9) | 352 | NM_001308305 | 116988 | ENSG00000133612 | AGAP3 |
| chr7 | 150812111 | exon (NM_001308305, exon 1 of 9) | 388 | NM_001308305 | 116988 | ENSG00000133612 | AGAP3 |
| chr7 | 150812201 | exon (NM_001308305, exon 1 of 9) | 478 | NM_001308305 | 116988 | ENSG00000133612 | AGAP3 |
| chr7 | 150812215 | exon (NM_001308305, exon 1 of 9) | 493 | NM_001308305 | 116988 | ENSG00000133612 | AGAP3 |
| chr7 | 150812398 | exon (NM_001308305, exon 1 of 9) | 675 | NM_001308305 | 116988 | ENSG00000133612 | AGAP3 |
| chr7 | 150812660 | exon (NM_001308305, exon 1 of 9) | 938 | NM_001308305 | 116988 | ENSG00000133612 | AGAP3 |
| chr7 | 157423275 | intron (NM_001308268, intron 14 of 22) | −56163 | NR_029689 | 406945 | ENSG00000207960 | MIR153-2 |
| chr7 | 157623246 | intron (NM_001308268, intron 12 of 22) | −24028 | NR_038966 | 100506585 | | LOC100506585 |
| chr8 | 46929775 | GSATX\|Satellite\|centr | 599488 | NR_146077 | 389652 | ENSG00000248498 | ASNSP1 |
| chr8 | 46935836 | GSATX\|Satellite\|centr | 593427 | NR_146077 | 389652 | ENSG00000248498 | ASNSP1 |
| chr8 | 46938035 | GSATX\|Satellite\|centr | 591228 | NR_146077 | 389652 | ENSG00000248498 | ASNSP1 |
| chr8 | 46938044 | GSATX\|Satellite\|centr | 591219 | NR_146077 | 389652 | ENSG00000248498 | ASNSP1 |
| chr8 | 46938657 | GSATX\|Satellite\|centr | 590606 | NR_146077 | 389652 | ENSG00000248498 | ASNSP1 |
| chr8 | 46938693 | GSATX\|Satellite\|centr | 590570 | NR_146077 | 380652 | ENSG00000248498 | ASNSP1 |
| chr8 | 46938702 | GSATX\|Satellite\|centr | 590561 | NR_146077 | 389652 | ENSG00000248498 | ASNSP1 |
| chr8 | 46940361 | FordPrefect\|DNA\|hAT-Tip100 | 588902 | NR_146077 | 389652 | ENSG00000248498 | ASNSP1 |
| chr8 | 46940406 | FordPrefect\|DNA\|hAT-Tip100 | 588857 | NR_146077 | 389652 | ENSG00000248498 | ASNSP1 |
| chr8 | 58062363 | LTR1D\|LTR\|ERV1 | −68470 | NR_038235 | 100507651 | ENSG00000253301 | LINC01606 |
| chr8 | 58062426 | LTR1D\|LTR\|ERV1 | −68407 | NR_038235 | 100507651 | ENSG00000253301 | LINC01606 |
| chr8 | 58062463 | LTR1D\|LTR\|ERV1 | −68370 | NR_038235 | 100507651 | ENSG00000253301 | LINC01606 |
| chr8 | 58096948 | AluSx1\|SINE\|Alu | −33885 | NR_038235 | 100507651 | ENSG00000253301 | LINC01606 |
| chr8 | 58115010 | Intergenic | −15823 | NR_038235 | 100507651 | ENSG00000253301 | LINC01606 |
| chr8 | 58115060 | Intergenic | −15773 | NR_038235 | 100507651 | ENSG00000253301 | LINC01606 |
| chr8 | 58115154 | MER50-int\|LTR\|ERV1 | −15679 | NR_038235 | 100507651 | ENSG00000253301 | LINC01606 |
| chr8 | 58115225 | MER50-int\|LTR\|ERV1 | −15608 | NR_038235 | 100507651 | ENSG00000253301 | LINC01606 |
| chr8 | 58115343 | MER50-int\|LTR\|ERV1 | −15490 | NR_038235 | 100507651 | ENSG00000253301 | LINC01606 |
| chr8 | 58115362 | MER50-int\|LTR\|ERV1 | −15471 | NR_038235 | 100507651 | ENSG00000253301 | LINC01606 |
| chr8 | 58122819 | MER50\|LTR\|ERV1 | −8014 | NR_038235 | 100507651 | ENSG00000253301 | LINC01606 |
| chr8 | 58122836 | MER50\|LTR\|ERV1 | −7997 | NR_038235 | 100507651 | ENSG00000253301 | LINC01606 |
| chr8 | 58122848 | MER50\|LTR\|ERV1 | −7985 | NR_038235 | 100507651 | ENSG00000253301 | LINC01606 |
| chr8 | 58122861 | MER50\|LTR\|ERV1 | −7972 | NR_038235 | 100507651 | ENSG00000253301 | LINC01606 |
| chr8 | 58123008 | Intergenic | −7825 | NR_038235 | 100507651 | ENSG00000253301 | LINC01606 |
| chr8 | 58126620 | MER50-int\|LTR\|ERV1 | −4213 | NR_038235 | 100507651 | ENSG00000253301 | LINC01606 |
| chr8 | 58126666 | MER50-int\|LTR\|ERV1 | −4167 | NR_038235 | 100507651 | ENSG00000253301 | LINC01606 |
| chr8 | 58126874 | MER50-int\|LTR\|ERV1 | −3959 | NR_038235 | 100507651 | ENSG00000253301 | LINC01606 |
| chr8 | 58127096 | MER50\|LTR\|ERV1 | −3737 | NR_038235 | 100507651 | ENSG00000253301 | LINC01606 |
| chr8 | 58127515 | MER50\|LTR\|ERV1 | −3318 | NR_038235 | 100507651 | ENSG00000253301 | LINC01606 |
| chr8 | 58127556 | MER50\|LTR\|ERV1 | −3277 | NR_038235 | 100507651 | ENSG00000253301 | LINC01606 |
| chr8 | 58127613 | MER50\|LTR\|ERV1 | −3220 | NR_038235 | 100507651 | ENSG00000253301 | LINC01606 |
| chr8 | 58130004 | promoter-TSS (NR_038235) | −829 | NR_038235 | 100507651 | ENSG00000253301 | LINC01606 |
| chr8 | 58130021 | promoter-TSS (NR_038235) | −812 | NR_038235 | 100507651 | ENSG00000253301 | LINC01606 |
| chr8 | 58130039 | promoter-TSS (NR_038235) | −794 | NR_038235 | 100507651 | ENSG00000253301 | LINC01606 |
| chr8 | 58131654 | non-coding (NR_038235, exon 3 of 10) | 821 | NR_038235 | 100507651 | ENSG00000253301 | LINC01606 |
| chr8 | 58131665 | non-coding (NR_038235, exon 3 of 10) | 832 | NR_038235 | 100507651 | ENSG00000253301 | LINC01606 |
| chr8 | 58131710 | MER50-int\|LTR\|ERV1 | 877 | NR_038235 | 100507651 | ENSG00000253301 | LINC01606 |
| chr8 | 58131715 | MER50-int\|LTR\|ERV1 | 882 | NR_038235 | 100507651 | ENSG00000253301 | LINC01606 |
| chr8 | 58131843 | MER50-int\|LTR\|ERV1 | 1010 | NR_038235 | 100507651 | ENSG00000253301 | LINC01606 |
| chr8 | 58131870 | MER50-int\|LTR\|ERV1 | 1037 | NR_038235 | 100507651 | ENSG00000253301 | LINC01606 |
| chr8 | 58133184 | intron (NR_038235, intron 5 of 9) | 2351 | NR_038235 | 100507651 | ENSG00000253301 | LINC01606 |
| chr8 | 58133215 | intron (NR_038235, intron 5 of 9) | 2382 | NR_038235 | 100507651 | ENSG00000253301 | LINC01606 |
| chr8 | 58133315 | intron (NR_038235, intron 5 of 9) | 2482 | NR_038235 | 100507651 | ENSG00000253301 | LINC01606 |
| chr8 | 58133743 | MER50\|LTR\|ERV1 | 2910 | NR_038235 | 100507651 | ENSG00000253301 | LINC01606 |
| chr8 | 58171196 | Intergenic | −2587 | NR_038874 | 286177 | ENSG00000253614 | LOC286177 |
| chr8 | 58171236 | Intergenic | −2545 | NR_038874 | 286177 | ENSG00000253614 | LOC286177 |
| chr8 | 58192020 | promoter-TSS (NR_026772) | −80 | NR_026772 | 26138 | ENSG00000215117 | LINC00588 |
| chr8 | 61777862 | exon (NM_001316690, exon 5 of 5) | 102443 | NR_034003 | 100130298 | | LOC100130298 |
| chr8 | 61821531 | Intergenic | 58774 | NR_034003 | 100130298 | | LOC100130298 |
| chr8 | 61821546 | Intergenic | 58759 | NR_034003 | 100130298 | | LOC100130298 |
| chr8 | 61821578 | C-rich\|Low_complexity\|Low_complexity | 58727 | NR_034003 | 100130298 | | LOC100130298 |
| chr8 | 73471538 | AluSx\|SINE\|Alu | 22080 | NM_004770 | 9312 | ENSG00000182674 | KCNB2 |
| chr8 | 73480053 | exon (NM_004770, exon 2 of 3) | 30595 | NM_004770 | 9312 | ENSG00000182674 | KCNB2 |
| chr8 | 73551842 | intron (NM_004770, intron 2 of 2) | −92436 | NR_110656 | 101926908 | ENSG00000253726 | LOC101926908 |
| chr8 | 73849013 | exon (NM_004770, exon 3 of 3) | −72084 | NM_017489 | 7013 | ENSG00000147601 | TERF1 |
| chr8 | 73849032 | exon (NM_004770, exon 3 of 3) | −72065 | NM_017489 | 7013 | ENSG00000147601 | TERF1 |
| chr8 | 73849038 | exon (NM_004770, exon 3 of 3) | −72059 | NM_017489 | 7013 | ENSG00000147601 | TERF1 |
| chr8 | 73849051 | exon (NM_004770, exon 3 of 3) | −72046 | NM_017489 | 7013 | ENSG00000147601 | TERF1 |
| chr8 | 73849060 | exon (NM_004770, exon 3 of 3) | −72037 | NM_017489 | 7013 | ENSG00000147601 | TERF1 |
| chr8 | 104117894 | Intergenic | −15364 | NR_145429 | 379034 | | LINC01181 |
| chr8 | 140929837 | intron (NM_001160372, intron 19 of 22) | 180795 | NR_144431 | 359809 | ENSG00000282164 | PEG13 |
| chr8 | 140930939 | intron (NM_001160372, intron 19 of 22) | 179693 | NR_144431 | 359809 | ENSG00000282164 | PEG13 |
| chr8 | 140931316 | intron (NM_001160372, intron 19 of 22) | 179316 | NR_144431 | 359809 | ENSG00000282164 | PEG13 |
| chr8 | 142367903 | exon (NM_005293, exon 2 of 2) | 9481 | NM_005293 | 2843 | ENSG00000204882 | GPR20 |
| chr8 | 142367951 | exon (NM_005293, exon 2 of 2) | 9433 | NM_005293 | 2843 | ENSG00000204882 | GPR20 |
| chr8 | 142367989 | exon (NM_005293, exon 2 of 2) | 9395 | NM_005293 | 2843 | ENSG00000204882 | GPR20 |
| chr9 | 117049844 | intron (NM_032888, intron 41 of 60) | −35457 | NM_000607 | 5004 | ENSG00000229314 | ORM1 |
| chr9 | 117049850 | intron (NM_032888, intron 41 of 60) | −35451 | NM_000607 | 5004 | ENSG00000229314 | ORM1 |
| chr9 | 117049862 | intron (NM_032888, intron 41 of 60) | −35439 | NM_000607 | 5004 | ENSG00000229314 | ORM1 |
| chr9 | 117049885 | intron (NM_032888, intron 41 of 60) | −35416 | NM_000607 | 5004 | ENSG00000229314 | ORM1 |
| chr9 | 127105722 | intron (NM_014397, intron 8 of 9) | −10028 | NR_027406 | 100129034 | | LOC100129034 |
| chr9 | 127105763 | intron (NM_014397, intron 8 of 9) | −9987 | NR_027406 | 100129034 | | LOC100129034 |
| chr9 | 129895923 | intron (NM_014636, intron 8 of 18) | −11011 | NM_012098 | 23452 | ENSG00000136859 | ANGPTL2 |
| chr9 | 136007375 | promoter-TSS (NM_001271774) | −833 | NM_001271774 | 5900 | ENSG00000160271 | RALGDS |
| chr9 | 136007484 | promoter-TSS (NM_001271774) | −942 | NM_001271774 | 5900 | ENSG00000160271 | RALGDS |
| chr9 | 136007594 | intron (NM_001042368, intron 1 of 17) | −1052 | NM_001271774 | 5900 | ENSG00000160271 | RALGDS |
| chr9 | 136007599 | intron (NM_001042368, intron 1 of 17) | −1057 | NM_001271774 | 5900 | ENSG00000160271 | RALGDS |
| chr9 | 136007615 | intron (NM_001042368, intron 1 of 17) | −1073 | NM_001271774 | 5900 | ENSG00000160271 | RALGDS |
| chr9 | 136007712 | intron (NM_001042368, intron 1 of 17) | −1170 | NM_001271774 | 5900 | ENSG00000160271 | RALGDS |
| chr9 | 136324027 | MIR\|SINE\|MIR | −1058 | NM_001242369 | 11094 | ENSG00000160325 | CACFD1 |
| chr9 | 136324037 | MIR\|SINE\|MIR | −1048 | NM_001242369 | 11094 | ENSG00000160325 | CACFD1 |
| chr9 | 136324064 | intron (NM_139026, intron 28 of 28) | −1021 | NM_001242369 | 11094 | ENSG00000160325 | CACFD1 |
| chr9 | 138688868 | Intergenic | 94839 | NM_020822 | 57582 | ENSG00000107147 | KCNT1 |
| chr9 | 138688876 | Intergenic | 94847 | NM_020822 | 57582 | ENSG00000107147 | KCNT1 |
| chr9 | 138688902 | Intergenic | 94873 | NM_020822 | 57582 | ENSG00000107147 | KCNT1 |
| chr9 | 138688936 | Intergenic | 94907 | NM_020822 | 57582 | ENSG00000107147 | KCNT1 |
| chr9 | 138689094 | Intergenic | 95066 | NM_020822 | 57582 | ENSG00000107147 | KCNT1 |
| chr9 | 138689107 | Intergenic | 95078 | NM_020822 | 57582 | ENSG00000107147 | KCNT1 |
| chr9 | 138689483 | Intergenic | 95454 | NM_020822 | 57582 | ENSG00000107147 | KCNT1 |
| chr9 | 138719312 | exon (NM_015447, exon 8 of 17) | 80078 | NM_015447 | 157922 | ENSG00000130559 | CAMSAP1 |
| chr9 | 138719386 | exon (NM_015447, exon 8 of 17) | 80004 | NM_015447 | 157922 | ENSG00000130559 | CAMSAP1 |
| chr9 | 139419669 | intron (NM_017617, intron 2 of 33) | −5593 | NR_039820 | 100616242 | ENSG00000263403 | MIR4673 |
| chr9 | 139419769 | intron (NM_017617, intron 2 of 33) | −5693 | NR_039820 | 100616242 | ENSG00000263403 | MIR4673 |
| chr9 | 139419814 | intron (NM_017617, intron 2 of 33) | −5738 | NR_039820 | 100616242 | ENSG00000263403 | MIR4673 |
| chr9 | 139419865 | intron (NM_017617, intron 2 of 33) | −5792 | NR_039820 | 100616242 | ENSG00000263403 | MIR4673 |
| chr9 | 139419924 | intron (NM_017617, intron 2 of 33) | −5848 | NR_039820 | 100616242 | ENSG00000263403 | MIR4673 |
| chr9 | 139419933 | intron (NM_017617, intron 2 of 33) | −5859 | NR_039820 | 100616242 | ENSG00000263403 | MIR4673 |
| chr9 | 139419956 | intron (NM_017617, intron 2 of 33) | −5880 | NR_039820 | 100616242 | ENSG00000263403 | MIR4673 |
| chr9 | 139420550 | intron (NM_017617, intron 2 of 33) | −6474 | NR_039820 | 100616242 | ENSG00000263403 | MIR4673 |
| chr9 | 139420561 | intron (NM_017617, intron 2 of 33) | −6485 | NR_039820 | 100616242 | ENSG00000263403 | MIR4673 |
| chr9 | 139420578 | intron (NM_017617, intron 2 of 33) | −6502 | NR_039820 | 100616242 | ENSG00000263403 | MIR4673 |
| chr9 | 139420602 | intron (NM_017617, intron 2 of 33) | −6526 | NR_039820 | 100616242 | ENSG00000263403 | MIR4673 |
| chr9 | 139420641 | intron (NM_017617, intron 2 of 33) | −6565 | NR_039820 | 100616242 | ENSG00000263403 | MIR4673 |
| chr9 | 139420862 | intron (NM_017617, intron 2 of 33) | −6786 | NR_039820 | 100616242 | ENSG00000263403 | MIR4673 |
| chr9 | 139420914 | intron (NM_017617, intron 2 of 33) | −6838 | NR_039820 | 100616242 | ENSG00000263403 | MIR4673 |
| chr9 | 139421144 | CpG | −7069 | NR_039820 | 100616242 | ENSG00000263403 | MIR4673 |
| chr9 | 139421220 | CpG | −7144 | NR_039820 | 100616242 | ENSG00000263403 | MIR4673 |
| chr9 | 139421241 | CpG | −7165 | NR_039820 | 100616242 | ENSG00000263403 | MIR4673 |
| chr9 | 139421257 | CpG | −7182 | NR_039820 | 100616242 | ENSG00000263403 | MIR4673 |
| chr9 | 139421267 | CpG | −7191 | NR_039820 | 100616242 | ENSG00000263403 | MIR4673 |
| chr9 | 139421321 | CpG | −7245 | NR_039820 | 100616242 | ENSG00000263403 | MIR4673 |
| chr9 | 139421342 | CpG | −7266 | NR_039820 | 100616242 | ENSG00000263403 | MIR4673 |
| chr9 | 139421362 | CpG | −7286 | NR_039820 | 100616242 | ENSG00000263403 | MIR4673 |
| chr9 | 139422178 | intron (NM_017617, intron 2 of 33) | −8102 | NR_039820 | 100616242 | ENSG00000263403 | MIR4673 |
| chr9 | 139422203 | intron (NM_017617, intron 2 of 33) | −8127 | NR_039820 | 100616242 | ENSG00000263403 | MIR4673 |
| chr9 | 139422218 | intron (NM_017617, intron 2 of 33) | −8143 | NR_039820 | 100616242 | ENSG00000263403 | MIR4673 |
| chr9 | 139422266 | intron (NM_017617, intron 2 of 33) | −8192 | NR_039820 | 100616242 | ENSG00000263403 | MIR4673 |
| chr9 | 139422290 | intron (NM_017617, intron 2 of 33) | −8214 | NR_039820 | 100616242 | ENSG00000263403 | MIR4673 |
| chr9 | 139422314 | intron (NM_017617, intron 2 of 33) | −8238 | NR_039820 | 100616242 | ENSG00000263403 | MIR4673 |
| chr10 | 23976532 | AluY\|SINE\|Alu | −7122 | NM_001098500 | 56243 | ENSG00000120549 | KIAA1217 |
| chr10 | 23976581 | AluY\|SINE\|Alu | −7073 | NM_001098500 | 56243 | ENSG00000120549 | KIAA1217 |
| chr10 | 72489844 | intron (NM_139155, intron 5 of 21) | 52483 | NR_134532 | 219793 | ENSG00000166220 | TBATA |
| chr10 | 72489870 | exon (NM_080722, exon 6 of 22) | 52454 | NR_134532 | 219793 | ENSG00000166220 | TBATA |
| chr10 | 72489889 | exon (NM_080722, exon 6 of 22) | 52438 | NR_134532 | 219793 | ENSG00000166220 | TBATA |
| chr10 | 72489949 | exon (NM_080722, exon 6 of 22) | 52378 | NR_134532 | 219793 | ENSG00000166220 | TBATA |
| chr10 | 72489967 | exon (NM_080722, exon 6 of 22) | 52360 | NR_134532 | 219793 | ENSG00000166220 | TBATA |
| chr10 | 72489983 | exon (NM_080722, exon 6 of 22) | 52344 | NR_134532 | 219793 | ENSG00000166220 | TBATA |
| chr10 | 119590052 | Intergenic | −216278 | NR_026939 | 255082 | ENSG00000177640 | CASC2 |
| chr10 | 134562610 | CpG | 37039 | NM_177400 | 84504 | ENSG00000148826 | NKX6-2 |
| chr10 | 134563052 | exon (NM_001321042, exon 7 of 13) | 36597 | NM_177400 | 84504 | ENSG00000148826 | NKX6-2 |
| chr10 | 134563060 | exon (NM_001321042, exon 7 of 13) | 36589 | NM_177400 | 84504 | ENSG00000148826 | NKX6-2 |
| chr10 | 134563098 | exon (NM_001321042, exon 7 of 13) | 36551 | NM_177400 | 84504 | ENSG00000148826 | NKX6-2 |
| chr10 | 134563109 | exon (NM_001321042, exon 7 of 13) | 36540 | NM_177400 | 84504 | ENSG00000148826 | NKX6-2 |
| chr10 | 134563144 | CpG-3491 | 36505 | NM_177400 | 84504 | ENSG00000148826 | NKX6-2 |
| chr10 | 134563149 | CpG-3491 | 36500 | NM_177400 | 84504 | ENSG00000148826 | NKX6-2 |
| chr10 | 134563186 | CpG-3491 | 36463 | NM_177400 | 84504 | ENSG00000148826 | NKX6-2 |
| chr10 | 134563199 | CpG-3491 | 36450 | NM_177400 | 84504 | ENSG00000148826 | NKX6-2 |
| chr11 | 2495501 | intron (NM_181798, intron 1 of 15) | 12819 | NM_181798 | 3784 | ENSG00000053918 | KCNQ1 |
| chr11 | 2495543 | intron (NM_181798, intron 1 of 15) | 12861 | NM_181798 | 3784 | ENSG00000053918 | KCNQ1 |
| chr11 | 2495585 | intron (NM_181798, intron 1 of 15) | 12903 | NM_181798 | 3784 | ENSG00000053918 | KCNQ1 |
| chr11 | 2495609 | intron (NM_181798, intron 1 of 15) | 12927 | NM_181798 | 3784 | ENSG00000053918 | KCNQ1 |
| chr11 | 68967954 | Intergenic | −28926 | NR_104161 | 338694 | ENSG00000261070 | LOC338694 |
| chr11 | 68967994 | Intergenic | −28966 | NR_104161 | 338694 | ENSG00000261070 | LOC338694 |
| chr11 | 68968016 | Intergenic | −28988 | NR_104161 | 338694 | ENSG00000261070 | LOC338694 |
| chr11 | 68968032 | Intergenic | −29004 | NR_104161 | 338694 | ENSG00000261070 | LOC338694 |
| chr11 | 68968042 | Intergenic | −29014 | NR_104161 | 338694 | ENSG00000261070 | LOC338694 |
| chr11 | 68968141 | Intergenic | −29115 | NR_104161 | 338694 | ENSG00000261070 | LOC338694 |
| chr11 | 68968151 | Intergenic | −29123 | NR_104161 | 338694 | ENSG00000261070 | LOC338694 |
| chr11 | 68968156 | Intergenic | −29128 | NR_104161 | 338694 | ENSG00000261070 | LOC338694 |
| chr11 | 68968182 | Intergenic | −29154 | NR_104161 | 338694 | ENSG00000261070 | LOC338694 |
| chr11 | 68968214 | Intergenic | −29186 | NR_104161 | 338694 | ENSG00000261070 | LOC338694 |
| chr11 | 68968240 | Intergenic | −29212 | NR_104161 | 338694 | ENSG00000261070 | LOC338694 |
| chr11 | 68968267 | Intergenic | −29240 | NR_104161 | 338694 | ENSG00000261070 | LOC338694 |
| chr11 | 70851529 | intron (NM_012309, intron 2 of 22) | 84311 | NM_012309 | 22941 | ENSG00000162105 | SHANK2 |
| chr11 | 70851574 | intron (NM_012309, intron 2 of 22) | 84266 | NM_012309 | 22941 | ENSG00000162105 | SHANK2 |
| chr11 | 70851603 | intron (NM_012309, intron 2 of 22) | 84237 | NM_012309 | 22941 | ENSG00000162105 | SHANK2 |
| chr11 | 70851619 | intron (NM_012309, intron 2 of 22) | 84221 | NM_012309 | 22941 | ENSG00000162105 | SHANK2 |
| chr11 | 70851638 | intron (NM_012309, intron 2 of 22) | 84202 | NM_012309 | 22941 | ENSG00000162105 | SHANK2 |
| chr11 | 70851713 | intron (NM_012309, intron 2 of 22) | 84127 | NM_012309 | 22941 | ENSG00000162105 | SHANK2 |
| chr11 | 70851722 | intron (NM_012309, intron 2 of 22) | 84118 | NM_012309 | 22941 | ENSG00000162105 | SHANK2 |
| chr11 | 70851730 | intron (NM_012309, intron 2 of 22) | 84110 | NM_012309 | 22941 | ENSG00000162105 | SHANK2 |
| chr11 | 70851827 | intron (NM_012309, intron 2 of 22) | 84013 | NM_012309 | 22941 | ENSG00000162105 | SHANK2 |
| chr11 | 70851836 | intron (NM_012309, intron 2 of 22) | 84004 | NM_012309 | 22941 | ENSG00000162105 | SHANK2 |
| chr11 | 70851859 | intron (NM_012309, intron 2 of 22) | 83981 | NM_012309 | 22941 | ENSG00000162105 | SHANK2 |
| chr11 | 70851868 | intron (NM_012309, intron 2 of 22) | 83972 | NM_012309 | 22941 | ENSG00000162105 | SHANK2 |
| chr11 | 70851883 | intron (NM_012309, intron 2 of 22) | 83957 | NM_012309 | 22941 | ENSG00000162105 | SHANK2 |
| chr11 | 70851889 | intron (NM_012309, intron 2 of 22) | 83951 | NM_012309 | 22941 | ENSG00000162105 | SHANK2 |
| chr11 | 70851914 | intron (NM_012309, intron 2 of 22) | 83926 | NM_012309 | 22941 | ENSG00000162105 | SHANK2 |
| chr11 | 70862301 | intron (NM_012309, intron 1 of 22) | 73539 | NM_012309 | 22941 | ENSG00000162105 | SHANK2 |
| chr11 | 70862379 | intron (NM_012309, intron 1 of 22) | 73461 | NM_012309 | 22941 | ENSG00000162105 | SHANK2 |
| chr11 | 70862397 | intron (NM_012309, intron 1 of 22) | 73443 | NM_012309 | 22941 | ENSG00000162105 | SHANK2 |
| chr11 | 70862405 | intron (NM_012309, intron 1 of 22) | 73435 | NM_012309 | 22941 | ENSG00000162105 | SHANK2 |
| chr11 | 111265961 | Intergenic | −15963 | NM_006235 | 5450 | ENSG00000110777 | POU2AE1 |
| chr11 | 111266000 | Intergenic | −16002 | NM_006235 | 5450 | ENSG00000110777 | POU2AE1 |
| chr11 | 111266014 | Intergenic | −16016 | NM_006235 | 5450 | ENSG00000110777 | POU2AE1 |
| chr11 | 111266031 | Intergenic | −16033 | NM_006235 | 5450 | ENSG00000110777 | POU2AE1 |
| chr11 | 111266119 | Intergenic | −16121 | NM_006235 | 5450 | ENSG00000110777 | POU2AF1 |
| chr11 | 115829517 | Intergenic | −198601 | NR_034148 | 283143 | | LINC00900 |
| chr11 | 115829550 | Intergenic | −198634 | NR_034148 | 283143 | | LINC00900 |
| chr11 | 115830020 | Intergenic | −199104 | NR_034148 | 283143 | | LINC00900 |
| chr11 | 115830200 | MIR3\|SINE\|MIR | −199284 | NR_034148 | 283143 | | LINC00900 |
| chr11 | 133902554 | non-coding (NR_027276, exon 1 of 3) | 389 | NR_027276 | 100128239 | ENSG00000204241 | LINC02731 |
| chr11 | 133902596 | non-coding (NR_027276, exon 1 of 3) | 431 | NR_027276 | 100128239 | ENSG00000204241 | LINC02731 |
| chr11 | 133902615 | non-coding (NR_027276, exon 1 of 3) | 450 | NR_027276 | 100128239 | ENSG00000204241 | LINC02731 |
| chr11 | 133902626 | non-coding (NR_027276, exon 1 of 3) | 461 | NR_027276 | 100128239 | ENSG00000204241 | LINC02731 |
| chr11 | 133902656 | non-coding (NR_027276, exon 1 of 3) | 491 | NR_027276 | 100128239 | ENSG00000204241 | LINC02731 |
| chr11 | 133902669 | non-coding (NR_027276, exon 1 of 3) | 504 | NR_027276 | 100128239 | ENSG00000204241 | LINC02731 |
| chr11 | 133902689 | non-coding (NR_027276, exon 1 of 3) | 524 | NR_027276 | 100128239 | ENSG00000204241 | LINC02731 |
| chr11 | 133902724 | non-coding (NR_027276, exon 1 of 3) | 561 | NR_027276 | 100128239 | ENSG00000204241 | LINC02731 |
| chr11 | 133902737 | non-coding (NR_027276, exon 1 of 3) | 572 | NR_027276 | 100128239 | ENSG00000204241 | LINC02731 |
| chr11 | 133902744 | non-coding (NR_027276, exon 1 of 3) | 579 | NR_027276 | 100128239 | ENSG00000204241 | LINC02731 |
| chr11 | 133902766 | intron (NR_027276, intron 1 of 2) | 601 | NR_027276 | 100128239 | ENSG00000204241 | LINC02731 |
| chr12 | 1957456 | L1MC5\|LINE\|L1 | 27727 | NM_001039029 | 654429 | ENSG00000166159 | LRTM2 |
| chr12 | 1957466 | L1MC5\|LINE\|L1 | 27737 | NM_001039029 | 654429 | ENSG00000166159 | LRTM2 |
| chr12 | 1957486 | L1MC5\|LINE\|L1 | 27757 | NM_001039029 | 654429 | ENSG00000166159 | LRTM2 |
| chr12 | 1957501 | L1MC5\|LINE\|L1 | 27772 | NM_001039029 | 654429 | ENSG00000166159 | LRTM2 |
| chr12 | 1957566 | L1MC5\|LINE\|L1 | 27837 | NM_001039029 | 654429 | ENSG00000166159 | LRTM2 |
| chr12 | 34488345 | GSATII\|Satellite\|centr | 312916 | NM_032834 | 84920 | ENSG00000139133 | ALG10 |
| chr12 | 34488352 | GSATII\|Satellite\|centr | 312923 | NM_032834 | 84920 | ENSG00000139133 | ALG10 |
| chr12 | 66111480 | AluY\|SINE\|Alu | −75330 | NR_120432 | 100507065 | | LOC100507065 |
| chr12 | 66111548 | LTR8A\|LTR\|ERV1 | −75398 | NR_120432 | 100507065 | | LOC100507065 |
| chr12 | 66114657 | Intergenic | −78507 | NR_120432 | 100507065 | | LOC100507065 |
| chr12 | 66114687 | Intergenic | −78537 | NR_120432 | 100507065 | | LOC100507065 |
| chr12 | 66114695 | Intergenic | −78545 | NR_120432 | 100507065 | | LOC100507065 |
| chr12 | 66262212 | intron (NR_158984, intron 1 of 2) | 13144 | NR_120478 | 100129940 | ENSG00000197301 | HMGA2-AS1 |
| chr12 | 66262228 | intron (NR_158984, intron 1 of 2) | 13126 | NR_120478 | 100129940 | ENSG00000197301 | HMGA2-AS1 |
| chr12 | 115109512 | 3′ UTR (NM_005996, exon 7 of 7) | 12466 | NM_005996 | 6926 | ENSG00000135111 | TBX3 |
| chr12 | 115109529 | 3′ UTR (NM_005996, exon 7 of 7) | 12449 | NM_005996 | 6926 | ENSG00000135111 | TBX3 |
| chr12 | 115109718 | exon (NM_005996, exon 7 of 7) | 12260 | NM_005996 | 6926 | ENSG00000135111 | TBX3 |
| chr12 | 115109960 | exon (NM_005996, exon 7 o7) | 12018 | NM_005996 | 6926 | ENSG00000135111 | TBX3 |
| chr12 | 115112024 | exon (NM_005996, exon 6 of 7) | 9954 | NM_005996 | 6926 | ENSG00000135111 | TBX3 |
| chr12 | 115112037 | exon (NM_005996, exon 6 of 7) | 9941 | NM_005996 | 6926 | ENSG00000135111 | TBX3 |
| chr12 | 124947867 | intron (NM_001206654, intron 7 of 47) | 104289 | NM_001206654 | 9612 | ENSG00000196498 | NCOR2 |
| chr12 | 124948139 | intron (NM_001206654, intron 7 of 47) | 104017 | NM_001206654 | 9612 | ENSG00000196498 | NCOR2 |
| chr13 | 114523664 | 3′ UTR (NM_000820, exon 15 of 15) | 5083 | NR_044995 | 650669 | | GAS6-AS1 |
| chr13 | 114523669 | 3′ UTR (NM_000820, exon 15 of 15) | 5088 | NR_044995 | 650669 | | GAS6-AS1 |
| chr13 | 114523717 | 3′ UTR (NM_000820, exon 15 of 15) | 5136 | NR_044995 | 650669 | | GAS6-AS1 |
| chr13 | 114523760 | 3′ UTR (NM_000820, exon 15 of 15) | 5179 | NR_044995 | 650669 | | GAS6-AS1 |
| chr13 | 114523787 | 3′ UTR (NM_000820, exon 15 of 15) | 5206 | NR_044995 | 650669 | | GAS6-AS1 |
| chr13 | 114523796 | 3′ UTR (NM_000820, exon 15 of 15) | 5215 | NR_044995 | 650669 | | GAS6-AS1 |
| chr13 | 114523819 | 3′ UTR (NM_000820, exon 15 of 15) | 5239 | NR_044995 | 650669 | | GAS6-AS1 |
| chr13 | 114523826 | 3′ UTR (NM_000820, exon 15 of 15) | 5245 | NR_044995 | 650669 | | GAS6-AS1 |
| chr13 | 114523875 | exon (NM_000820, exon 15 of 15) | 5294 | NR_044995 | 650669 | | GAS6-AS1 |
| chr13 | 114523883 | exon (NM_000820, exon 15 of 15) | 5302 | NR_044995 | 650669 | | GAS6-AS1 |
| chr13 | 114523896 | exon (NM_000820, exon 15 of 15) | 5316 | NR_044995 | 650669 | | GAS6-AS1 |
| chr13 | 114523904 | exon (NM_000820, exon 15 of 15) | 5323 | NR_044995 | 650669 | | GAS6-AS1 |
| chr13 | 114523923 | exon (NM_000820, exon 15 of 15) | 5342 | NR_044995 | 650669 | | GAS6-AS1 |
| chr13 | 114523948 | exon (NM_000820, exon 15 of 15) | 5368 | NR_044995 | 650669 | | GAS6-AS1 |
| chr13 | 114523958 | exon (NM_000820, exon 15 of 15) | 5378 | NR_044995 | 650669 | | GAS6-AS1 |
| chr13 | 114523970 | exon (NM_000820, exon 15 of 15) | 5389 | NR_044995 | 650669 | | GAS6-AS1 |
| chr13 | 114523982 | exon (NM_000820, exon 15 of 15) | 5401 | NR_044995 | 650669 | | GAS6-AS1 |
| chr13 | 114523996 | CpG | 5415 | NR_044995 | 650669 | | GAS6-AS1 |
| chr13 | 114524029 | CpG | 5448 | NR_044995 | 650669 | | GAS6-AS1 |
| chr13 | 114524093 | CpG | 5512 | NR_044995 | 650669 | | GAS6-AS1 |
| chr13 | 114524130 | CpG | 5549 | NR_044995 | 650669 | | GAS6-AS1 |
| chr13 | 114524137 | CpG | 5558 | NR_044995 | 650669 | | GAS6-AS1 |
| chr13 | 114524149 | CpG | 5568 | NR_044995 | 650669 | | GAS6-AS1 |
| chr13 | 114524157 | CpG | 5576 | NR_044995 | 650669 | | GAS6-AS1 |
| chr13 | 114524420 | intron (NM_000820, intron 14 of 14) | 5839 | NR_044995 | 650669 | | GAS6-AS1 |
| chr13 | 114524429 | intron (NM_000820, intron 14 of 14) | 5848 | NR_044995 | 650669 | | GAS6-AS1 |
| chr13 | 114524439 | intron (NM_000820, intron 14 of 14) | 5858 | NR_044995 | 650669 | | GAS6-AS1 |
| chr13 | 114524473 | intron (NM_000820, intron 14 of 14) | 5892 | NR_044995 | 650669 | | GAS6-AS1 |
| chr13 | 114524509 | intron (NM_000820, intron 14 of 14) | 5928 | NR_044995 | 650669 | | GAS6-AS1 |
| chr13 | 114524517 | intron (NM_000820, intron 14 of 14) | 5936 | NR_044995 | 650669 | | GAS6-AS1 |
| chr13 | 114524583 | intron (NM_000820, intron 14 of 14) | 6003 | NR_044995 | 650669 | | GAS6-AS1 |
| chr14 | 20684518 | Intergenic | −7349 | NM_001004480 | 122748 | ENSG00000176219 | OR11H6 |
| chr14 | 20684528 | Intergenic | −7339 | NM_001004480 | 122748 | ENSG00000176219 | OR11H6 |
| chr14 | 76957115 | intron (NM_004452, intron 7 of 10) | 119427 | NM_004452 | 2103 | ENSG00000119715 | ESRRB |
| chr14 | 76957138 | intron (NM_004452, intron 7 of 10) | 119450 | NM_004452 | 2103 | ENSG00000119715 | ESRRB |
| chr14 | 76957152 | intron (NM_004452, intron 7 of 10) | 119464 | NM_004452 | 2103 | ENSG00000119715 | ESRRB |
| chr14 | 76957199 | intron (NM_004452, intron 7 of 10) | 119511 | NM_004452 | 2103 | ENSG00000119715 | ESRRB |
| chr14 | 93423461 | intron (NM_001142593, intron 8 of 10) | 33968 | NM_001275 | 1113 | ENSG00000100604 | CHGA |
| chr14 | 93423503 | intron (NM_001142593, intron 8 of 10) | 34010 | NM_001275 | 1113 | ENSG00000100604 | CHGA |
| chr14 | 93423558 | intron (NM_001142593, intron 8 of 10) | 34065 | NM_001275 | 1113 | ENSG00000100604 | CHGA |
| chr14 | 93425210 | MIRb\|SINE\|MIR | 35717 | NM_001275 | 1113 | ENSG00000100604 | CHGA |
| chr14 | 93425284 | MIRb\|SINE\|MIR | 35791 | NM_001275 | 1113 | ENSG00000100604 | CHGA |
| chr14 | 93425331 | MIRb\|SINE\|MIR | 35838 | NM_001275 | 1113 | ENSG00000100604 | CHGA |
| chr14 | 93425520 | intron (NM_001142593, intron 7 of 10) | 36027 | NM_001275 | 1113 | ENSG00000100604 | CHGA |
| chr14 | 93425529 | intron (NM_001142593, intron 7 of 10) | 36036 | NM_001275 | 1113 | ENSG00000100604 | CHGA |
| chr14 | 93425655 | intron (NM_001142593, intron 7 of 10) | 36162 | NM_001275 | 1113 | ENSG00000100604 | CHGA |
| chr14 | 93425687 | intron (NM_001142593, intron 7 of 10) | 36194 | NM_001275 | 1113 | ENSG00000100604 | CHGA |
| chr14 | 93425735 | intron (NM_001142593, intron 7 of 10) | 36242 | NM_001275 | 1113 | ENSG00000100604 | CHGA |
| chr14 | 93425741 | intron (NM_001142593, intron 7 of 10) | 36249 | NM_001275 | 1113 | ENSG00000100604 | CHGA |
| chr14 | 93426221 | intron (NM_001142593, intron 7 of 10) | 36728 | NM_001275 | 1113 | ENSG00000100604 | CHGA |
| chr14 | 93426229 | intron (NM_001142593, intron 7 of 10) | 36737 | NM_001275 | 1113 | ENSG00000100604 | CHGA |
| chr14 | 93426247 | intron (NM_001142593, intron 7 of 10) | 36754 | NM_001275 | 1113 | ENSG00000100604 | CHGA |
| chr14 | 93426257 | intron (NM_001142593, intron 7 of 10) | 36764 | NM_001275 | 1113 | ENSG00000100604 | CHGA |
| chr14 | 93426336 | intron (NM_001142593, intron 7 of 10) | 36843 | NM_001275 | 1113 | ENSG00000100604 | CHGA |
| chr14 | 93426381 | intron (NM_001142593, intron 7 of 10) | 36888 | NM_001275 | 1113 | ENSG00000100604 | CHGA |
| chr14 | 93426456 | intron (NM_001142593, intron 7 of 10) | 36963 | NM_001275 | 1113 | ENSG00000100604 | CHGA |
| chr14 | 93426481 | intron (NM_001142593, intron 7 of 10) | 36988 | NM_001275 | 1113 | ENSG00000100604 | CHGA |
| chr14 | 93426499 | intron (NM_001142593, intron 7 of 10) | 37006 | NM_001275 | 1113 | ENSG00000100604 | CHGA |
| chr14 | 93426875 | MIRb\|SINE\|MIR | 37382 | NM_001275 | 1113 | ENSG00000100604 | CHGA |
| chr14 | 93426916 | MIRb\|SINE\|MIR | 37423 | NM_001275 | 1113 | ENSG00000100604 | CHGA |
| chr14 | 101158277 | Intergenic | 34674 | NR_024096 | 283601 | ENSG00000196273 | LINC00523 |
| chr14 | 101158294 | Intergenic | 34691 | NR_024096 | 283601 | ENSG00000196273 | LINC00523 |
| chr14 | 101159404 | L2a\|LINE\|L2 | −33796 | NM_003836 | 8788 | ENSG00000185559 | DLK1 |
| chr14 | 101159430 | L2a\|LINE\|L2 | −33769 | NM_003836 | 8788 | ENSG00000185559 | DLKl |
| chr14 | 101159551 | L2a\|LINE\|L2 | −33647 | NM_003836 | 8788 | ENSG00000185559 | DLK1 |
| chr14 | 101250153 | Intergenic | −30673 | NR_039729 | 100616495 | ENSG00000266461 | MIR2392 |
| chr14 | 101250165 | Intergenic | −30661 | NR_039729 | 100616495 | ENSG00000266461 | MIR2392 |
| chr14 | 101250240 | Charlie13b\|DNA\|hAT-Charlie | −30584 | NR_039729 | 100616495 | ENSG00000266461 | MIR2392 |
| chr14 | 101250254 | Charlie13b\|DNA\|hAT-Charlie | −30571 | NR_039729 | 100616495 | ENSG00000266461 | MIR2392 |
| chr14 | 101250279 | Charlie13b\|DNA\|hAT-Charlie | −30547 | NR_039729 | 100616495 | ENSG00000266461 | MIR2392 |
| chr14 | 101640895 | Intergenic | 53705 | NR_146490 | 101929422 | ENSG00000259004 | LINC02285 |
| chr14 | 101640910 | Intergenic | 53719 | NR_146490 | 101929422 | ENSG00000259004 | LINC02285 |
| chr14 | 101640942 | Intergenic | 53751 | NR_146490 | 101929422 | ENSG00000259004 | LINC02285 |
| chr14 | 101640959 | Intergenic | 53768 | NR_146490 | 101929422 | ENSG00000259004 | LINC02285 |
| chr14 | 105347685 | intron (NM_001112726, intron 6 of 18) | 16036 | NR_162135 | 113219454 | | MIR12121 |
| chr14 | 105347700 | intron (NM_001112726, intron 6 of 18) | 16051 | NR_162135 | 113219454 | | MIR12121 |
| chr14 | 105347712 | intron (NM_001112726, intron 6 of 18) | 16063 | NR_162135 | 113219454 | | MIR12121 |
| chr14 | 105347802 | intron (NM_001112726, intron 6 of 18) | 16153 | NR_162135 | 113219454 | | MIR12121 |
| chr14 | 105347810 | intron (NM_001112726, intron 6 of 18) | 16161 | NR_162135 | 113219454 | | MIR12121 |
| chr14 | 105347834 | intron (NM_001112726, intron 6 of 18) | 16185 | NR_162135 | 113219454 | | MIR12121 |
| chr14 | 105347851 | intron (NM_001112726, intron 6 of 18) | 16202 | NR_162135 | 113219454 | | MIR12121 |
| chr14 | 105347961 | intron (NM_001112726, intron 6 of 18) | 16312 | NR_162135 | 113219454 | | MIR12121 |
| chr15 | 22559959 | intron (NR_033735, intron 1 of 2) | 13396 | NR_033735 | 646396 | | REREP3 |
| chr15 | 22559966 | intron (NR_033735, intron 1 of 2) | 13403 | NR_033735 | 646396 | | REREP3 |
| chr15 | 22567152 | non-coding (NR_033735, exon 2 of 3) | 20589 | NR_033735 | 646396 | | REREP3 |
| chr15 | 22567172 | non-coding (NR_033735, exon 2 of 3) | 20609 | NR_033735 | 646396 | | REREP3 |
| chr15 | 22571751 | TTS (NR_033735) | 25188 | NR_033735 | 646396 | | REREP3 |
| chr15 | 22571783 | TTS (NR_033735) | 25220 | NR_033735 | 646396 | | REREP3 |
| chr15 | 22581845 | AluSc8\|SINE\|Alu | 35282 | NR_033735 | 646396 | | REREP3 |
| chr15 | 22581981 | AluSc8\|SINE\|Alu | 35418 | NR_033735 | 646396 | | REREP3 |
| chr15 | 74521143 | Intergenic | −7522 | NM_025055 | 80125 | ENSG00000140481 | CCDC33 |
| chr15 | 74521156 | Intergenic | −7509 | NM_025055 | 80125 | ENSG00000140481 | CCDC33 |
| chr15 | 74521297 | Intergenic | −7368 | NM_025055 | 80125 | ENSG00000140481 | CCDC33 |
| chr15 | 74521344 | Intergenic | −7321 | NM_025055 | 80125 | ENSG00000140481 | CCDC33 |
| chr15 | 74521357 | Intergenic | −7308 | NM_025055 | 80125 | ENSG00000140481 | CCDC33 |
| chr15 | 74521364 | Intergenic | −7301 | NM_025055 | 80125 | ENSG00000140481 | CCDC33 |
| chr15 | 94613752 | AmnSINE1\|SINE\|Deu | 37412 | NR_120320 | 101927112 | ENSG00000259724 | LINC01581 |
| chr15 | 94613766 | AmnSINE1\|SINE\|Deu | 37399 | NR_120320 | 101927112 | ENSG00000259724 | LINC01581 |
| chr15 | 94613787 | AmnSINE1\|SINE\|Deu | 37378 | NR_120320 | 101927112 | ENSG00000259724 | LINC01581 |
| chr15 | 94613823 | AmnSINE1\|SINE\|Deu | 37341 | NR_120320 | 101927112 | ENSG00000259724 | LINC01581 |
| chr15 | 94613901 | intron (NR_120320, intron 2 of 8) | 37264 | NR_120320 | 101927112 | ENSG00000259724 | LINC01581 |
| chr17 | 915606 | MER102a\|DNA\|hAT-Charlie | 10191 | NR_036148 | 100422835 | ENSG00000264429 | MIR3183 |
| chr17 | 915643 | MER102a\|DNA\|hAT-Charlie | 10154 | NR_036148 | 100422835 | ENSG00000264429 | MIR3183 |
| chr17 | 915806 | CpG | 9989 | NR_036148 | 100422835 | ENSG00000264429 | MIR3183 |
| chr17 | 915890 | CpG | 9907 | NR_036148 | 100422835 | ENSG00000264429 | MIR3183 |
| chr17 | 915912 | CpG | 9885 | NR_036148 | 100422835 | ENSG00000264429 | MIR3183 |
| chr17 | 7010397 | exon (NM_001201352, exon 7 of 9) | 7230 | NM_080914 | 433 | ENSG00000161944 | ASGR2 |
| chr17 | 7010445 | exon (NM_001201352, exon 7 of 9) | 7182 | NM_080914 | 433 | ENSG00000161944 | ASGR2 |
| chr17 | 7010504 | intron (NM_001181, intron 6 of 8) | 7123 | NM_080914 | 433 | ENSG00000161944 | ASGR2 |
| chr17 | 7010533 | intron (NM_001181, intron 6 of 8) | 7094 | NM_080914 | 433 | ENSG00000161944 | ASGR2 |
| chr17 | 7010548 | exon (NM_001201352, exon 6 of 9) | 7079 | NM_080914 | 433 | ENSG00000161944 | ASGR2 |
| chr17 | 7010630 | exon (NM_001201352, exon 6 of 9) | 6997 | NM_080914 | 433 | ENSG00000161944 | ASGR2 |
| chr17 | 7010663 | intron (NM_001181, intron 5 of 8) | 6964 | NM_080914 | 433 | ENSG00000161944 | ASGR2 |
| chr17 | 10728383 | 3′ UTR (NM_001101387, exon 2 of 2) | 13019 | NM_001101387 | 644139 | ENSG00000233670 | PIRT |
| chr17 | 10740512 | intron (NM_001101387, intron 1 of 1) | 890 | NM_001101387 | 644139 | ENSG00000233670 | PIRT |
| chr17 | 10740558 | intron (NM_001101387, intron 1 of 1) | 842 | NM_001101387 | 644139 | ENSG00000233670 | PIRT |
| chr17 | 10740569 | intron (NM_001101387, intron 1 of 1) | 833 | NM_001101387 | 644139 | ENSG00000233670 | PIRT |
| chr17 | 10740581 | intron (NM_001101387, intron 1 of 1) | 821 | NM_001101387 | 644139 | ENSG00000233670 | PIRT |
| chr17 | 10740595 | intron (NM_001101387, intron 1 of 1) | 807 | NM_001101387 | 644139 | ENSG00000233670 | PIRT |
| chr17 | 17677711 | intron (NM_030665, intron 2 of 5) | 5130 | NR_024007 | 140771 | ENSG00000226746 | SMCR5 |
| chr17 | 17677757 | intron (NM_030665, intron 2 of 5) | 5084 | NR_024007 | 140771 | ENSG00000226746 | SMCR5 |
| chr17 | 17677887 | intron (NM_030665, intron 2 of 5) | 4954 | NR_024007 | 140771 | ENSG00000226746 | SMCR5 |
| chr17 | 21286388 | intron (NM_021012, intron 1 of 2) | 6721 | NM_021012 | 3768 | ENSG00000184185 | KCNJ12 |
| chr17 | 21286444 | intron (NM_021012, intron 1 of 2) | 6777 | NM_021012 | 3768 | ENSG00000184185 | KCNJ12 |
| chr17 | 21286486 | intron (NM_021012, intron 1 of 2) | 6819 | NM_021012 | 3768 | ENSG00000184185 | KCNJ12 |
| chr17 | 21286510 | intron (NM_021012, intron 1 of 2) | 6846 | NM_021012 | 3768 | ENSG00000184185 | KCNJ12 |
| chr17 | 21286534 | intron (NM_021012, intron 1 of 2) | 6867 | NM_021012 | 3768 | ENSG00000184185 | KCNJ12 |
| chr17 | 21286708 | intron (NM_021012, intron 1 of 2) | 7042 | NM_021012 | 3768 | ENSG00000184185 | KCNJ12 |
| chr17 | 21286741 | intron (NM_021012, intron 1 of 2) | 7074 | NM_021012 | 3768 | ENSG00000184185 | KCNJ12 |
| chr17 | 21286747 | intron (NM_021012, intron 1 of 2) | 7080 | NM_021012 | 3768 | ENSG00000184185 | KCNJ12 |
| chr17 | 28299552 | intron (NR_026738, intron 4 of 15) | 30902 | NM_198529 | 374786 | ENSG00000176927 | EFCAB5 |
| chr17 | 28299565 | intron (NR_026738, intron 4 of 15) | 30915 | NM_198529 | 374786 | ENSG00000176927 | EFCAB5 |
| chr17 | 28299571 | intron (NR_026738, intron 4 of 15) | 30922 | NM_198529 | 374786 | ENSG00000176927 | EFCAB5 |
| chr17 | 28299598 | intron (NR_026738, intron 4 of 15) | 30948 | NM_198529 | 374786 | ENSG00000176927 | EFCAB5 |
| chr17 | 28299697 | intron (NR_026738, intron 4 of 15) | 31047 | NM_198529 | 374786 | ENSG00000176927 | EFCAB5 |
| chr17 | 71715903 | Intergenic | −18088 | NM_001278587 | 100134391 | | LOC100134391 |
| chr17 | 71715940 | Intergenic | −18051 | NM_001278587 | 100134391 | | LOC100134391 |
| chr17 | 71715947 | Intergenic | −18044 | NM_001278587 | 100134391 | | LOC100134391 |
| chr17 | 71715958 | Intergenic | −18031 | NM_001278587 | 100134391 | | LOC100134391 |
| chr17 | 71716005 | Intergenic | −17986 | NM_001278587 | 100134391 | | LOC100134391 |
| chr17 | 71743873 | intron (NM_001278587, intron 2 of 5) | 9882 | NM_001278587 | 100134391 | | LOC100134391 |
| chr17 | 71744668 | TTS (NR_027146) | 10677 | NM_001278587 | 100134391 | | LOC100134391 |
| chr17 | 71744685 | TTS (NR_027146) | 10694 | NM_001278587 | 100134391 | | LOC100134391 |
| chr17 | 71744723 | TTS (NR_027146) | 10732 | NM_001278587 | 100134391 | | LOC100134391 |
| chr17 | 71744739 | TTS (NR_027146) | 10748 | NM_001278587 | 100134391 | | LOC100134391 |
| chr17 | 78079606 | exon (NM_000152, exon 3 of 20) | 4228 | NM_001079803 | 2548 | ENSG00000171298 | GAA |
| chr17 | 78079613 | exon (NM_000152, exon 3 of 20) | 4235 | NM_001079803 | 2548 | ENSG00000171298 | GAA |
| chr17 | 78079629 | exon (NM_000152, exon 3 of 20) | 4251 | NM_001079803 | 2548 | ENSG00000171298 | GAA |
| chr17 | 78079641 | exon (NM_000152, exon 3 of 20) | 4263 | NM_001079803 | 2548 | ENSG00000171298 | GAA |
| chr17 | 78079653 | exon (NM_000152, exon 3 of 20) | 4275 | NM_001079803 | 2548 | ENSG00000171298 | GAA |
| chr17 | 78395359 | intron (NR_148041, intron 1 of 6) | 2143 | NR_039883 | 100616359 | ENSG00000264961 | MIR4730 |
| chr17 | 78395376 | intron (NR_148041, intron 1 of 6) | 2160 | NR_039883 | 100616359 | ENSG00000264961 | MIR4730 |
| chr17 | 80314127 | Intergenic | −2994 | NM_207459 | 400629 | ENSG00000182459 | TEX19 |
| chr17 | 80314154 | Intergenic | −2967 | NM_207459 | 400629 | ENSG00000182459 | TEX19 |
| chr17 | 80914812 | intron (NR_135465, intron 10 of 13) | 50050 | NR_135466 | 146712 | ENSG00000175711 | B3GNTL1 |
| chr17 | 80915007 | intron (NR_135465, intron 10 of 13) | 49855 | NR_135466 | 146712 | ENSG00000175711 | B3GNTL1 |
| chr17 | 80915042 | intron (NR_135465, intron 10 of 13) | 49820 | NR_135466 | 146712 | ENSG00000175711 | B3GNTL1 |
| chr17 | 80915160 | intron (NR_135465, intron 9 of 13) | 49702 | NR_135466 | 146712 | ENSG00000175711 | B3GNTL1 |
| chr17 | 80915190 | intron (NR_135465, intron 9 of 13) | 49672 | NR_135466 | 146712 | ENSG00000175711 | B3GNTL1 |
| chr17 | 80915238 | exon (NM_001009905, exon 9 of 13) | 49624 | NR_135466 | 146712 | ENSG00000175711 | B3GNTL1 |
| chr17 | 80915248 | exon (NM_001009905, exon 9 of 13) | 49614 | NR_135466 | 146712 | ENSG00000175711 | B3GNTL1 |
| chr17 | 80915270 | exon (NM_001009905, exon 9 of 13) | 49591 | NR_135466 | 146712 | ENSG00000175711 | B3GNTL1 |
| chr17 | 80915289 | exon (NM_001009905, exon 9 of 13) | 49573 | NR_135466 | 146712 | ENSG00000175711 | B3GNTL1 |
| chr17 | 80915308 | exon (NM_001009905, exon 9 of 13) | 49554 | NR_135466 | 146712 | ENSG00000175711 | B3GNTL1 |
| chr18 | 8659649 | CpG | 47966 | NR_120598 | 100287082 | ENSG00000265962 | GACAT2 |
| chr18 | 24348426 | Intergenic | −64826 | NR_024259 | 728606 | ENSG00000265369 | PCAT18 |
| chr18 | 25366256 | Intergenic | 250347 | NM_001308176 | 1000 | ENSG00000170558 | CDH2 |
| chr18 | 25366262 | Intergenic | 250341 | NM_001308176 | 1000 | ENSG00000170558 | CDH2 |
| chr18 | 25366300 | Intergenic | 250303 | NM_001308176 | 1000 | ENSG00000170558 | CDH2 |
| chr18 | 25366309 | Intergenic | 250294 | NM_001308176 | 1000 | ENSG00000170558 | CDH2 |
| chr18 | 25442175 | Intergenic | 174428 | NM_001308176 | 1000 | ENSG00000170558 | CDH2 |
| chr18 | 25442224 | Intergenic | 174379 | NM_001308176 | 1000 | ENSG00000170558 | CDH2 |
| chr18 | 25442243 | Intergenic | 174360 | NM_001308176 | 1000 | ENSG00000170558 | CDH2 |
| chr18 | 68166868 | Intergenic | −115043 | NR_110764 | 101060542 | ENSG00000266278 | LINC01910 |
| chr18 | 68166878 | Intergenic | −115055 | NR_110764 | 101060542 | ENSG00000266278 | LINC01910 |
| chr18 | 77320496 | Intergenic | −18784 | NR_148949 | 284240 | | LOC284240 |
| chr18 | 77320538 | Intergenic | −18742 | NR_148949 | 284240 | | LOC284240 |
| chr18 | 77320583 | Intergenic | −18697 | NR_148949 | 284240 | | LOC284240 |
| chr18 | 77320593 | Intergenic | −18687 | NR_148949 | 284240 | | LOC284240 |
| chr18 | 77320606 | Intergenic | −18674 | NR_148949 | 284240 | | LOC284240 |
| chr18 | 77320672 | Intergenic | −18608 | NR_148949 | 284240 | | LOC284240 |
| chr18 | 77320695 | Intergenic | −18585 | NR_148949 | 284240 | | LOC284240 |
| chr18 | 77320711 | Intergenic | −18569 | NR_148949 | 284240 | | LOC284240 |
| chr18 | 77320739 | Intergenic | −18541 | NR_148949 | 284240 | | LOC284240 |
| chr19 | 883734 | intron (NM_005481, intron 6 of 15) | 9451 | NM_005481 | 10025 | ENSG00000175221 | MED16 |
| chr19 | 883775 | intron (NM_005481, intron 6 of 15) | 9410 | NM_005481 | 10025 | ENSG00000175221 | MED16 |
| chr19 | 883830 | intron (NM_005481, intron 6 of 15) | 9355 | NM_005481 | 10025 | ENSG00000175221 | MED16 |
| chr19 | 883873 | intron (NM_005481, intron 6 of 15) | 9310 | NM_005481 | 10025 | ENSG00000175221 | MED16 |
| chr19 | 883885 | intron (NM_005481, intron 6 of 15) | 9300 | NM_005481 | 10025 | ENSG00000175221 | MED16 |
| chr19 | 883994 | intron (NM_005481, intron 6 of 15) | 9191 | NM_005481 | 10025 | ENSG00000175221 | MED16 |
| chr19 | 884032 | intron (NM_005481, intron 6 of 15) | 9153 | NM_005481 | 10025 | ENSG00000175221 | MED16 |
| chr19 | 884042 | intron (NM_005481, intron 6 of 15) | 9143 | NM_005481 | 10025 | ENSG00000175221 | MED16 |
| chr19 | 884057 | intron (NM_005481, intron 6 of 15) | 9128 | NM_005481 | 10025 | ENSG00000175221 | MED16 |
| chr19 | 884140 | intron (NM_005481, intron 6 of 15) | 9045 | NM_005481 | 10025 | ENSG00000175221 | MED16 |
| chr19 | 884147 | intron (NM_005481, intron 6 of 15) | 9038 | NM_005481 | 10025 | ENSG00000175221 | MED16 |
| chr19 | 884172 | intron (NM_005481, intron 6 of 15) | 9013 | NM_005481 | 10025 | ENSG00000175221 | MED16 |
| chr19 | 884233 | intron (NM_005481, intron 6 of 15) | 8952 | NM_005481 | 10025 | ENSG00000175221 | MED16 |
| chr19 | 884267 | intron (NM_005481, intron 6 of 15) | 8918 | NM_005481 | 10025 | ENSG00000175221 | MED16 |
| chr19 | 884346 | intron (NM_005481, intron 6 of 15) | 8839 | NM_005481 | 10025 | ENSG00000175221 | MED16 |
| chr19 | 2299058 | MIR3\|SINE\|MIR | 9093 | NM_001101391 | 645191 | ENSG00000220008 | LINGO3 |
| chr19 | 2299180 | intron (NM_001101391, intron 1 of 1) | 8970 | NM_001101391 | 645191 | ENSG00000220008 | LINGO3 |
| chr19 | 2299218 | intron (NM_001101391, intron 1 of 1) | 8933 | NM_001101391 | 645191 | ENSG00000220008 | LINGO3 |
| chr19 | 2299353 | intron (NM_001101391, intron 1 of 1) | 8798 | NM_001101391 | 645191 | ENSG00000220008 | LINGO3 |
| chr19 | 2299390 | intron (NM_001101391, intron 1 of 1) | 8761 | NM_001101391 | 645191 | ENSG00000220008 | LINGO3 |
| chr19 | 4566603 | CpG | −6909 | NM_032108 | 10501 | ENSG00000167680 | SEMA6B |
| chr19 | 4566636 | CpG | −6942 | NM_032108 | 10501 | ENSG00000167680 | SEMA6B |
| chr19 | 4566642 | CpG | −6951 | NM_032108 | 10501 | ENSG00000167680 | SEMA6B |
| chr19 | 4566653 | CpG | −6961 | NM_032108 | 10501 | ENSG00000167680 | SEMA6B |
| chr19 | 9994121 | intron (NM_058164, intron 1 of 5) | −25272 | NM_001304348 | 93145 | ENSG00000105088 | OLFM2 |
| chr19 | 9994137 | intron (NM_058164, intron 1 of 5) | −25288 | NM_001304348 | 93145 | ENSG00000105088 | OLFM2 |
| chr19 | 9994208 | intron (NM_058164, intron 1 of 5) | −25359 | NM_001304348 | 93145 | ENSG00000105088 | OLFM2 |
| chr19 | 9994219 | intron (NM_058164, intron 1 of 5) | −25370 | NM_001304348 | 93145 | ENSG00000105088 | OLFM2 |
| chr19 | 9994333 | intron (NM_058164, intron 1 of 5) | −25484 | NM_001304348 | 93145 | ENSG00000105088 | OLFM2 |
| chr19 | 9994349 | intron (NM_058164, intron 1 of 5) | −25500 | NM_001304348 | 93145 | ENSG00000105088 | OLFM2 |
| chr19 | 13108021 | intron (NM_001365982, intron 1 of 8) | 1734 | NM_001365982 | 4784 | ENSG00000008441 | NFIX |
| chr19 | 13108640 | intron (NM_001365982, intron 1 of 8) | 2353 | NM_001365982 | 4784 | ENSG00000008441 | NFIX |
| chr19 | 13112778 | CpG | 6491 | NM_001365982 | 4784 | ENSG00000008441 | NFIX |
| chr19 | 13616660 | intron (NM_001127222, intron 1 of 46) | 612 | NM_001127221 | 773 | ENSG00000141837 | CACNA1A |
| chr19 | 13616810 | exon (NM_001127221, exon 1 of 47) | 462 | NM_001127221 | 773 | ENSG00000141837 | CACNA1A |
| chr19 | 13616818 | exon (NM_001127221, exon 1 of 47) | 454 | NM_001127221 | 773 | ENSG00000141837 | CACNA1A |
| chr19 | 13616834 | exon (NM_001127221, exon 1 of 47) | 436 | NM_001127221 | 773 | ENSG00000141837 | CACNA1A |
| chr19 | 13616868 | exon (NM_001127221, exon 1 of 47) | 403 | NM_001127221 | 773 | ENSG00000141837 | CACNA1A |
| chr19 | 13616879 | exon (NM_001127221, exon 1 of 47) | 393 | NM_001127221 | 773 | ENSG00000141837 | CACNA1A |
| chr19 | 13616906 | exon (NM_001127221, exon 1 of 47) | 366 | NM_001127221 | 773 | ENSG00000141837 | CACNA1A |
| chr19 | 13616911 | exon (NM_001127221, exon 1 of 47) | 361 | NM_001127221 | 773 | ENSG00000141837 | CACNA1A |
| chr19 | 13617010 | exon (NM_001127221, exon 1 of 47) | 262 | NM_001127221 | 773 | ENSG00000141837 | CACNA1A |
| chr19 | 13617018 | exon (NM_001127221, exon 1 of 47) | 254 | NM_001127221 | 773 | ENSG00000141837 | CACNA1A |
| chr19 | 13617024 | exon (NM_001127221, exon 1 of 47) | 248 | NM_001127221 | 773 | ENSG00000141837 | CACNA1A |
| chr19 | 13617029 | exon (NM_001127221, exon 1 of 47) | 243 | NM_001127221 | 773 | ENSG00000141837 | CACNA1A |
| chr19 | 13617058 | 5′ UTR (NM_001127221, exon 1 of 47) | 214 | NM_001127221 | 773 | ENSG00000141837 | CACNA1A |
| chr19 | 13617065 | 5′ UTR (NM_001127221, exon 1 of 47) | 207 | NM_001127221 | 773 | ENSG00000141837 | CACNA1A |
| chr19 | 13617070 | 5′ UTR (NM_001127221, exon 1 of 47) | 199 | NM_001127221 | 773 | ENSG00000141837 | CACNA1A |
| chr19 | 13617082 | 5′ UTR (NM_001127221, exon 1 of 47) | 190 | NM_001127221 | 773 | ENSG00000141837 | CACNA1A |
| chr19 | 13617090 | 5′ UTR (NM_001127221, exon 1 of 47) | 172 | NM_001127221 | 773 | ENSG00000141837 | CACNA1A |
| chr19 | 13617128 | 5′ UTR (NM_001127221, exon 1 of 47) | 144 | NM_001127221 | 773 | ENSG00000141837 | CACNA1A |
| chr19 | 13617140 | 5′ UTR (NM_001127221, exon 1 of 47) | 130 | NM_001127221 | 773 | ENSG00000141837 | CACNA1A |
| chr19 | 13617157 | 5′ UTR (NM_001127221, exon 1 of 47) | 115 | NM_001127221 | 773 | ENSG00000141837 | CACNA1A |
| chr19 | 13617163 | 5′ UTR (NM_001127221, exon 1 of 47) | 109 | NM_001127221 | 773 | ENSG00000141837 | CACNA1A |
| chr19 | 13617169 | 5′ UTR (NM_001127221, exon 1 of 47) | 103 | NM_001127221 | 773 | ENSG00000141837 | CACNA1A |
| chr19 | 13617176 | promoter-TSS (NM_001127221) | 96 | NM_001127221 | 773 | ENSG00000141837 | CACNA1A |
| chr19 | 13617194 | promoter-TSS (NM_001127221) | 75 | NM_001127221 | 773 | ENSG00000141837 | CACNA1A |
| chr19 | 13617208 | promoter-TSS (NM_001127221) | 64 | NM_001127221 | 773 | ENSG00000141837 | CACNA1A |
| chr19 | 13617218 | promoter-TSS (NM_001127221) | 54 | NM_001127221 | 773 | ENSG00000141837 | CACNA1A |
| chr19 | 13617233 | promoter-TSS (NM_001127221) | 39 | NM_001127221 | 773 | ENSG00000141837 | CACNA1A |
| chr19 | 13617241 | promoter-TSS (NM_001127221) | 31 | NM_001127221 | 773 | ENSG00000141837 | CACNA1A |
| chr19 | 13617250 | promoter-TSS (NM_001127221) | 22 | NM_001127221 | 773 | ENSG00000141837 | CACNA1A |
| chr19 | 13617265 | promoter-TSS (NM_001127221) | 7 | NM_001127221 | 773 | ENSG00000141837 | CACNA1A |
| chr19 | 13617288 | promoter-TSS (NM_001127221) | 3 | NM_001174080 | 773 | ENSG00000141837 | CACNA1A |
| chr19 | 13617302 | promoter-TSS (NM_001127221) | −11 | NM_001127222 | 773 | ENSG00000141837 | CACNA1A |
| chr19 | 13617315 | promoter-TSS (NM_001127221) | −24 | NM_001127222 | 773 | ENSG00000141837 | CACNA1A |
| chr19 | 13617321 | promoter-TSS (NM_001127221) | −30 | NM_001127222 | 773 | ENSG00000141837 | CACNA1A |
| chr19 | 13617328 | promoter-TSS (NM_001127221) | −37 | NM_001127222 | 773 | ENSG00000141837 | CACNA1A |
| chr19 | 13617336 | promoter-TSS (NM_001127221) | −45 | NM_001127222 | 773 | ENSG00000141837 | CACNA1A |
| chr19 | 13617364 | promoter-TSS (NM_001127221) | −73 | NM_001127222 | 773 | ENSG00000141837 | CACNA1A |
| chr19 | 13617380 | promoter-TSS (NM_001127221) | −89 | NM_001127222 | 773 | ENSG00000141837 | CACNA1A |
| chr19 | 13617419 | promoter-TSS (NM_001127221) | −128 | NM_001127222 | 773 | ENSG00000141837 | CACNA1A |
| chr19 | 13617465 | promoter-TSS (NM_001127221) | −174 | NM_001127222 | 773 | ENSG00000141837 | CACNA1A |
| chr19 | 13617483 | promoter-TSS (NM_001127221) | −195 | NM_001127222 | 773 | ENSG00000141837 | CACNA1A |
| chr19 | 13617503 | promoter-TSS (NM_001127221) | −212 | NM_001127222 | 773 | ENSG00000141837 | CACNA1A |
| chr19 | 13617512 | promoter-TSS (NM_001127221) | −221 | NM_001127222 | 773 | ENSG00000141837 | CACNA1A |
| chr19 | 13617517 | promoter-TSS (NM_001127221) | −226 | NM_001127222 | 773 | ENSG00000141837 | CACNA1A |
| chr19 | 13617541 | promoter-TSS (NM_001127221) | −250 | NM_001127222 | 773 | ENSG00000141837 | CACNA1A |
| chr19 | 13618356 | Intergenic | −1065 | NM_001127222 | 773 | ENSG00000141837 | CACNA1A |
| chr19 | 13618391 | Intergenic | −1101 | NM_001127222 | 773 | ENSG00000141837 | CACNA1A |
| chr19 | 13700379 | MER58B\|DNA\|hAT-Charlie | −83088 | NM_001127222 | 773 | ENSG00000141837 | CACNA1A |
| chr19 | 18836689 | intron (NM_001098482, intron 1 of 14) | 42201 | NM_001098482 | 23373 | ENSG00000105662 | CRTC1 |
| chr19 | 18837043 | intron (NM_001098482, intron 1 of 14) | 42556 | NM_001098482 | 23373 | ENSG00000105662 | CRTC1 |
| chr19 | 18837091 | intron (NM_001098482, intron 1 of 14) | 42603 | NM_001098482 | 23373 | ENSG00000105662 | CRTC1 |
| chr19 | 18837098 | intron (NM_001098482, intron 1 of 14) | 42610 | NM_001098482 | 23373 | ENSG00000105662 | CRTC1 |
| chr19 | 18837105 | intron (NM_001098482, intron 1 of 14) | 42617 | NM_001098482 | 23373 | ENSG00000105662 | CRTC1 |
| chr19 | 34181556 | intron (NM_001127895, intron 3 of 4) | 6124 | NM_022467 | 64377 | ENSG00000124302 | CHST8 |
| chr19 | 34181606 | intron (NM_001127895, intron 3 of 4) | 6174 | NM_022467 | 64377 | ENSG00000124302 | CHST8 |
| chr19 | 34181636 | intron (NM_001127895, intron 3 of 4) | 6204 | NM_022467 | 64377 | ENSG00000124302 | CHST8 |
| chr19 | 38527887 | L2b\|LINE\|L2 | −125847 | NR_145800 | 109623473 | | SNORD152 |
| chr19 | 38527913 | L2b\|LINE\|L2 | −125821 | NR_145800 | 109623473 | | SNORD152 |
| chr19 | 38527932 | L2b\|LINE\|L2 | −125802 | NR_145800 | 109623473 | | SNORD152 |
| chr19 | 38528016 | L2b\|LINE\|L2 | −125718 | NR_145800 | 109623473 | | SNORD152 |
| chr19 | 38528031 | L2b\|LINE\|L2 | −125701 | NR_145800 | 109623473 | | SNORD152 |
| chr19 | 38684235 | exon (NM_015073, exon 18 of 22) | 30501 | NR_145800 | 109623473 | | SNORD152 |
| chr19 | 38684327 | exon (NM_015073, exon 18 of 22) | 30561 | NM_001135155 | 8193 | ENSG00000011332 | DPF1 |
| chr19 | 38684338 | exon (NM_015073, exon 18 of 22) | 30550 | NM_001135155 | 8193 | ENSG00000011332 | DPF1 |
| chr19 | 38684363 | exon (NM_015073, exon 18 of 22) | 30522 | NM_001135155 | 8193 | ENSG00000011332 | DPF1 |
| chr19 | 48699558 | exon (NM_199341, exon 4 of 4) | 25623 | NM_199341 | 374920 | ENSG00000185453 | ZSWIM9 |
| chr19 | 48699578 | exon (NM_199341, exon 4 of 4) | 25643 | NM_199341 | 374920 | ENSG00000185453 | ZSWIM9 |
| chr19 | 48699588 | exon (NM_199341, exon 4 of 4) | 25653 | NM_199341 | 374920 | ENSG00000185453 | ZSWIM9 |
| chr19 | 48699615 | exon (NM_199341, exon 4 of 4) | 25680 | NM_199341 | 374920 | ENSG00000185453 | ZSWIM9 |
| chr19 | 48699628 | exon (NM_199341, exon 4 of 4) | 25693 | NM_199341 | 374920 | ENSG00000185453 | ZSWIM9 |
| chr19 | 48699667 | exon (NM_199341, exon 4 of 4) | 25733 | NM_199341 | 374920 | ENSG00000185453 | ZSWIM9 |
| chr19 | 48699717 | exon (NM_199341, exon 4 of 4) | 25782 | NM_199341 | 374920 | ENSG00000185453 | ZSWIM9 |
| chr19 | 55870392 | exon (NM_001145402, exon 9 of 11) | 4234 | NM_001145402 | 284418 | ENSG00000180043 | FAM71E2 |
| chr19 | 55870402 | exon (NM_001145402, exon 9 of 11) | 4224 | NM_001145402 | 284418 | ENSG00000180043 | FAM71E2 |
| chr19 | 55870407 | exon (NM_001145402, exon 9 of 11) | 4219 | NM_001145402 | 284418 | ENSG00000180043 | FAM71E2 |
| chr19 | 55870425 | exon (NM_001145402, exon 9 of 11) | 4201 | NM_001145402 | 284418 | ENSG00000180043 | FAM71E2 |
| chr19 | 55870439 | exon (NM_001145402, exon 9 of 11) | 4187 | NM_001145402 | 284418 | ENSG00000180043 | FAM71E2 |
| chr19 | 55870456 | exon (NM_001145402, exon 9 of 11) | 4170 | NM_001145402 | 284418 | ENSG00000180043 | FAM71E2 |
| chr19 | 55870462 | exon (NM_001145402, exon 9 of 11) | 4164 | NM_001145402 | 284418 | ENSG00000180043 | FAM71E2 |
| chr19 | 55870471 | exon (NM_001145402, exon 9 of 11) | 4155 | NM_001145402 | 284418 | ENSG00000180043 | FAM71E2 |
| chr19 | 55870886 | exon (NM_001145402, exon 9 of 11) | 3740 | NM_001145402 | 284418 | ENSG00000180043 | FAM71E2 |
| chr20 | 20180688 | intron (NM_015585, intron 17 of 26) | 143401 | NM_001167816 | 26074 | ENSG00000089101 | CFAP61 |
| chr20 | 20180732 | intron (NM_015585, intron 17 of 26) | 143444 | NM_001167816 | 26074 | ENSG00000089101 | CFAP61 |
| chr20 | 20188098 | intron (NM_015585, intron 17 of 26) | 150810 | NM_001167816 | 26074 | ENSG00000089101 | CFAP61 |
| chr20 | 20188151 | intron (NM_015585, intron 17 of 26) | 150863 | NM_001167816 | 26074 | ENSG00000089101 | CFAP61 |
| chr20 | 20206077 | intron (NM_015585, intron 17 of 26) | −142669 | NM_002196 | 3642 | ENSG00000173404 | INSM1 |
| chr20 | 20232312 | exon (NM_015585, exon 20 of 27) | −116434 | NM_002196 | 3642 | ENSG00000173404 | INSM1 |
| chr20 | 20232336 | exon (NM_015585, exon 20 of 27) | −116410 | NM_002196 | 3642 | ENSG00000173404 | INSM1 |
| chr20 | 20232350 | exon (NM_015585, exon 20 of 27) | −116394 | NM_002196 | 3642 | ENSG00000173404 | INSM1 |
| chr20 | 20232360 | exon (NM_015585, exon 20 of 27) | −116386 | NM_002196 | 3642 | ENSG00000173404 | INSM1 |
| chr20 | 20232370 | exon (NM_015585, exon 20 of 27) | −116376 | NM_002196 | 3642 | ENSG00000173404 | INSM1 |
| chr20 | 20232391 | exon (NM_015585, exon 20 of 27) | −116355 | NM_002196 | 3642 | ENSG00000173404 | INSM1 |
| chr20 | 20376947 | intron (NM_020343, intron 39 of 39) | 28201 | NM_002196 | 3642 | ENSG00000173404 | INSM1 |
| chr20 | 20376962 | intron (NM_020343, intron 39 of 39) | 28216 | NM_002196 | 3642 | ENSG00000173404 | INSM1 |
| chr20 | 20377074 | intron (NM_020343, intron 39 of 39) | 28328 | NM_002196 | 3642 | ENSG00000173404 | INSM1 |
| chr20 | 20377089 | intron (NM_020343, intron 39 of 39) | 28343 | NM_002196 | 3642 | ENSG00000173404 | INSM1 |
| chr20 | 20377110 | intron (NM_020343, intron 39 of 39) | 28364 | NM_002196 | 3642 | ENSG00000173404 | INSM1 |
| chr20 | 20377122 | intron (NM_020343, intron 39 of 39) | 28376 | NM_002196 | 3642 | ENSG00000173404 | INSM1 |
| chr20 | 20377130 | intron (NM_020343, intron 39 of 39) | 28384 | NM_002196 | 3642 | ENSG00000173404 | INSM1 |
| chr20 | 20377162 | intron (NM_020343, intron 39 of 39) | 28416 | NM_002196 | 3642 | ENSG00000173404 | INSM1 |
| chr20 | 20377169 | intron (NM_020343, intron 39 of 39) | 28423 | NM_002196 | 3642 | ENSG00000173404 | INSM1 |
| chr20 | 20377206 | intron (NM_020343, intron 39 of 39) | 28460 | NM_002196 | 3642 | ENSG00000173404 | INSM1 |
| chr20 | 20392139 | intron (NM_020343, intron 38 of 39) | 43396 | NM_002196 | 3642 | ENSG00000173404 | INSM1 |
| chr20 | 20392184 | intron (NM_020343, intron 38 of 39) | 43438 | NM_002196 | 3642 | ENSG00000173404 | INSM1 |
| chr20 | 20392215 | intron (NM_020343, intron 38 of 39) | 43469 | NM_002196 | 3642 | ENSG00000173404 | INSM1 |
| chr20 | 20392408 | intron (NM_020343, intron 38 of 39) | 43662 | NM_002196 | 3642 | ENSG00000173404 | INSM1 |
| chr20 | 20421295 | intron (NM_020343, intron 37 of 39) | 72549 | NM_002196 | 3642 | ENSG00000173404 | INSM1 |
| chr20 | 20421314 | intron (NM_020343, intron 37 of 39) | 72568 | NM_002196 | 3642 | ENSG00000173404 | INSM1 |
| chr20 | 20421345 | intron (NM_020343, intron 37 of 39) | 72599 | NM_002196 | 3642 | ENSG00000173404 | INSM1 |
| chr20 | 20421371 | intron (NM_020343, intron 37 of 39) | 72627 | NM_002196 | 3642 | ENSG00000173404 | INSM1 |
| chr20 | 20421396 | intron (NM_020343, intron 37 of 39) | 72650 | NM_002196 | 3642 | ENSG00000173404 | INSM1 |
| chr20 | 20421420 | intron (NM_020343, intron 37 of 39) | 72675 | NM_002196 | 3642 | ENSG00000173404 | INSM1 |
| chr20 | 20421459 | intron (NM_020343, intron 37 of 39) | 72713 | NM_002196 | 3642 | ENSG00000173404 | INSM1 |
| chr20 | 20421486 | intron (NM_020343, intron 37 of 39) | 72740 | NM_002196 | 3642 | ENSG00000173404 | INSM1 |
| chr20 | 31129481 | intron (NM_001256798, intron 1 of 10) | 31721 | NR_110619 | 101929698 | ENSG00000277301 | LOC01929698 |
| chr20 | 36923352 | Intergenic | −9198 | NM_001725 | 671 | ENSG00000101425 | BPI |
| chr20 | 36923369 | MIRb\|SINE\|MIR | −9178 | NM_001725 | 671 | ENSG00000101425 | BPI |
| chr20 | 36923397 | MIRb\|SINE\|MIR | −9153 | NM_001725 | 671 | ENSG00000101425 | BPI |
| chr20 | 61013054 | intron (NR_033263, intron 7 of 7) | −10461 | NM_080833 | 140893 | ENSG00000130701 | RBBP8NL |
| chr20 | 61013094 | intron (NR_033263, intron 7 of 7) | −10501 | NM_080833 | 140893 | ENSG00000130701 | RBBP8NL |
| chr20 | 61013113 | intron (NR_033263, intron 7 of 7) | −10520 | NM_080833 | 140893 | ENSG00000130701 | RBBP8NL |
| chr20 | 61013148 | intron (NR_033263, intron 7 o7) | −10555 | NM_080833 | 140893 | ENSG00000130701 | RBBP8NL |
| chr20 | 61013167 | intron (NR_033263, intron 7 o7) | −10574 | NM_080833 | 140893 | ENSG00000130701 | RBBP8NL |
| chr20 | 61013187 | intron (NR_033263, intron 7 of 7) | −10596 | NM_080833 | 140893 | ENSG00000130701 | RBBP8NL |
| chr20 | 61013226 | intron (NR_033263, intron 7 of 7) | −10633 | NM_080833 | 140893 | ENSG00000130701 | RBBP8NL |
| chr20 | 61013242 | intron (NR_033263, intron 7 of 7) | −10649 | NM_080833 | 140893 | ENSG00000130701 | RBBP8NL |
| chr20 | 61013251 | intron (NR_033263, intron 7 of 7) | −10658 | NM_080833 | 140893 | ENSG00000130701 | RBBP8NL |
| chr20 | 61013263 | intron (NR_033263, intron 7 of 7) | −10670 | NM_080833 | 140893 | ENSG00000130701 | RBBP8NL |
| chr20 | 61013303 | intron (NR_033263, intron 7 of 7) | −10710 | NM_080833 | 140893 | ENSG00000130701 | RBBP8NL |
| chr20 | 61013339 | intron (NR_033263, intron 7 of 7) | −10747 | NM_080833 | 140893 | ENSG00000130701 | RBBP8NL |
| chr20 | 61013474 | intron (NR_033263, intron 7 of 7) | −10881 | NM_080833 | 140893 | ENSG00000130701 | RBBP8NL |
| chr20 | 61013515 | intron (NR_033263, intron 7 of 7) | −10922 | NM_080833 | 140893 | ENSG00000130701 | RBBP8NL |
| chr20 | 61020520 | intron (NR_033263, intron 7 of 7) | −17927 | NM_080833 | 140893 | ENSG00000130701 | RBBP8NL |
| chr20 | 61021747 | intron (NR_033263, intron 7 of 7) | −19154 | NM_080833 | 140893 | ENSG00000130701 | RBBP8NL |
| chr21 | 43442046 | promoter-TSS (NR_119385) | −65 | NR_119385 | 150142 | ENSG00000237232 | ZNF295-AS1 |
| chr21 | 43442055 | promoter-TSS (NR_119385) | −56 | NR_119385 | 150142 | ENSG00000237232 | ZNF295-AS1 |
| chr21 | 43442083 | promoter-TSS (NR_119385) | −28 | NR_119385 | 150142 | ENSG00000237232 | ZNF295-AS1 |
| chr21 | 43442252 | promoter-TSS (NR_027273) | −124 | NR_027273 | 150142 | ENSG00000237232 | ZNF295-AS1 |
| chr21 | 43442338 | promoter-TSS (NR_027273) | −38 | NR_027273 | 150142 | ENSG00000237232 | ZNF295-AS1 |
| chr21 | 43442384 | promoter-TSS (NR_027273) | 8 | NR_027273 | 150142 | ENSG00000237232 | ZNF295-AS1 |
| chr21 | 44836763 | exon (NM_001320643, exon 14 of 14) | 10246 | NM_173354 | 150094 | ENSG00000142178 | SIK1 |
| chr21 | 44836797 | exon (NM_001320643, exon 14 of 14) | 10212 | NM_173354 | 150094 | ENSG00000142178 | SIK1 |
| chr21 | 144836823 | exon (NM_001320643, exon 14 of 14) | 10186 | NM_173354 | 150094 | ENSG00000142178 | SIK1 |
| chr21 | 44836842 | exon (NM_001320643, exon 14 of 14) | 10167 | NM_173354 | 150094 | ENSG00000142178 | SIK1 |
| chr21 | 44836924 | exon (NM_001320643, exon 14 of 14) | 10085 | NM_173354 | 150094 | ENSG00000142178 | SIK1 |
| chr21 | 44836936 | exon (NM_001320643, exon 14 of 14) | 10073 | NM_173354 | 150094 | ENSG00000142178 | SIK1 |
| chr21 | 44836971 | exon (NM_001320643, exon 14 of 14) | 10038 | NM_173354 | 150094 | ENSG00000142178 | SIK1 |
| chr21 | 44837026 | CpG | 9980 | NM_173354 | 150094 | ENSG00000142178 | SIK1 |
| chr21 | 44837044 | CpG | 9965 | NM_173354 | 150094 | ENSG00000142178 | SIK1 |
| chr21 | 44837052 | CpG | 9957 | NM_173354 | 150094 | ENSG00000142178 | SIK1 |
| chr21 | 44837060 | CpG | 9949 | NM_173354 | 150094 | ENSG00000142178 | SIK1 |
| chr21 | 44837074 | CpG | 9933 | NM_173354 | 150094 | ENSG00000142178 | SIK1 |
| chr21 | 44837115 | CpG | 9894 | NM_173354 | 150094 | ENSG00000142178 | SIK1 |
| chr21 | 44837132 | CpG | 9876 | NM_173354 | 150094 | ENSG00000142178 | SIK1 |
| chr21 | 44837157 | CpG | 9852 | NM_173354 | 150094 | ENSG00000142178 | SIK1 |
| chr21 | 145653981 | intron (NM_015259, intron 4 of 6) | 6843 | NM_001283050 | 23308 | ENSG00000160223 | ICOSLG |
| chr21 | 45654156 | intron (NM_015259, intron 4 of 6) | 6668 | NM_001283050 | 23308 | ENSG00000160223 | ICOSLG |
| chr22 | 47444997 | intron (NM_014346, intron 11 of 12) | −133061 | NR_122047 | 642757 | ENSG00000280080 | TBC1D22A-AS1 |
| chr22 | 47445037 | intron (NM_014346, intron 11 of 12) | −133101 | NR_122047 | 642757 | ENSG00000280080 | TBC1D22A-AS1 |
| chr22 | 47447406 | intron (NM_014346, intron 11 of 12) | −135470 | NR_122047 | 642757 | ENSG00000280080 | TBC1D22A-AS1 |
| chr22 | 47447603 | intron (NM_014346, intron 11 of 12) | −135667 | NR_122047 | 642757 | ENSG00000280080 | TBC1D22A-AS1 |
| chrX | 104458760 | L1PA8\|LINE\|L1 | 6615 | NM_031274 | 56157 | ENSG00000268629 | TEX13A |
| chrX | 104692650 | AluSq2\|SINE\|Alu | −227275 | NM_001291277 | 56157 | ENSG00000268629 | TEX13A |
| chrX | 104804571 | AlnSq2\|SINE\|Alu | −261959 | NM_198465 | 203447 | ENSG00000123572 | NRK |
| chrX | 104891135 | LTR17\|LTR\|ERV1 | −175395 | NM_198465 | 203447 | ENSG00000123572 | NRK |
| chrX | 105016634 | AluSg\|SINE\|Alu | −49896 | NM_198465 | 203447 | ENSG00000123572 | NRK |
| chrX | 105016672 | AluSg\|SINE\|Alu | −49858 | NM_198465 | 203447 | ENSG00000123572 | NRK |
| chrX | 128926432 | exon (NM_018990, exon 5 of 8) | 12509 | NM_018990 | 54440 | ENSG00000122122 | SASH3 |
| chr2 | 209103955 | intron (NM_005896, intron 8 of 9) | 15057 | NM_001282387 | 3417 | ENSG00000138413 | IDH1 |
| chr4 | 106155150 | exon (NM_001127208, exon 3 of 11) | 87323 | NM_001127208 | 54790 | ENSG00000168769 | TET2 |
| chr4 | 106155913 | exon (NM_001127208, exon 3 of 11) | 88086 | NM_001127208 | 54790 | ENSG00000168769 | TET2 |
| chr4 | 106156460 | exon (NM_001127208, exon 3 of 11) | 88633 | NM_001127208 | 54790 | ENSG00000168769 | TET2 |
| chr4 | 106156742 | exon (NM_001127208, exon 3 of 11) | 88915 | NM_001127208 | 54790 | ENSG00000168769 | TET2 |
| chr4 | 106157415 | exon (NM_001127208, exon 3 of 11) | 89588 | NM_001127208 | 54790 | ENSG00000168769 | TET2 |
| chr4 | 106164901 | TTS (NM_017628) | 97074 | NM_001127208 | 54790 | ENSG00000168769 | TET2 |
| chr4 | 106196236 | exon (NM_001127208, exon 11 of 11) | 77787 | NR_126420 | 104384744 | | TET2-AS1 |
| chr7 | 33732332 | Intergenic | −212165 | NM_133468 | 168667 | ENSG00000164619 | BMPER |
| chr7 | 140453119 | exon (NM_004333, exon 15 of 18) | −56257 | NR_024454 | 100134713 | ENSG00000240889 | NDUFB2-AS1 |
| chr7 | 140481387 | exon (NM_004333, exon 11 of 18) | −84525 | NR_024454 | 100134713 | ENSG00000240889 | NDUFB2-AS1 |
| chr7 | 140481426 | exon (NM_004333, exon 11 of 18) | −84564 | NR_024454 | 100134713 | ENSG00000240889 | NDUFB2-AS1 |
| chr7 | 140500421 | intron (NR_148928, intron 7 of 17) | −103559 | NR_024454 | 100134713 | ENSG00000240889 | NDUFB2-AS1 |
| chr7 | 140507748 | exon (NM_004333, exon 5 of 18) | −110886 | NR_024454 | 100134713 | ENSG00000240889 | NDUFB2-AS1 |
| chr7 | 140507954 | intron (NM_001354609, intron 4 of 18) | −111092 | NR_024454 | 100134713 | ENSG00000240889 | NDUFB2-AS1 |
| chr7 | 140624441 | exon (NM_004333, exon 1 of 18) | 273 | NM_001354609 | 673 | ENSG00000157764 | BRAF |
| chr14 | 38061176 | exon (NM_004496, exon 2 of 2) | 3252 | NM_004496 | 3169 | ENSG00000129514 | FOXA1 |
| chr14 | 38061319 | exon (NM_004496, exon 2 of 2) | 3120 | NM_004496 | 3169 | ENSG00000129514 | FOXA1 |
| chr14 | 38061830 | exon (NM_004496, exon 2 of 2) | 2609 | NM_004496 | 3169 | ENSG00000129514 | FOXA1 |
| chr17 | 7572948 | exon (NM_000546, exon 11 of 11) | 5599 | NM_001276698 | 7157 | ENSG00000141510 | TP53 |
| chr17 | 7573967 | exon (NM_000546, exon 10 of 11) | 4554 | NM_001276698 | 7157 | ENSG00000141510 | TP53 |
| chr17 | 7576524 | exon (NM_001126117, exon 6 of 8) | 2022 | NM_001276698 | 7157 | ENSG00000141510 | TP53 |
| chr17 | 7576838 | exon (NM_001126117, exon 5 of 8) | 1672 | NM_001276698 | 7157 | ENSG00000141510 | TP53 |
| chr17 | 7577020 | exon (NM_001126117, exon 4 of 8) | 1474 | NM_001276698 | 7157 | ENSG00000141510 | TP53 |
| chr17 | 7577483 | exon (NM_001126117, exon 3 of 8) | 1008 | NM_001276698 | 7157 | ENSG00000141510 | TP53 |
| chr17 | 7578161 | exon (NM_001126117, exon 2 of 8) | 337 | NM_001276698 | 7157 | ENSG00000141510 | TP53 |
| chr17 | 7578373 | promoter-TSS (NM_001276697) | 90 | NM_001276698 | 7157 | ENSG00000141510 | TP53 |
| chr17 | 7579297 | promoter-TSS (NM_001126117) | −519 | NM_001126116 | 7157 | ENSG00000141510 | TP53 |
| chr17 | 7579393 | promoter-TSS (NM_001126117) | −601 | NM_001126116 | 7157 | ENSG00000141510 | TP53 |
| chr17 | 7579506 | promoter-TSS (NM_001126117) | −710 | NM_001126116 | 7157 | ENSG00000141510 | TP53 |
| chr17 | 7579701 | promoter-TSS (NM_001126117) | −905 | NM_001126116 | 7157 | ENSG00000141510 | TP53 |
| chr17 | 7579846 | exon (NM_000546, exon 2 of 11) | −1050 | NM_001126116 | 7157 | ENSG00000141510 | TP53 |
| chr17 | 47696409 | exon (NM_001007228, exon 5 of 10) | 7515 | NM_001370732 | 8405 | ENSG00000121067 | SPOP |
| chr17 | 47696673 | exon (NM_001007228, exon 4 of 10) | 7263 | NM_001370732 | 8405 | ENSG00000121067 | SPOP |
| chr20 | 31375142 | exon (NM_175848, exon 6 of 22) | 7499 | NM_175850 | 1789 | ENSG00000088305 | DNMT3B |
| chr20 | 31380384 | intron (NM_175849, intron 8 of 19) | 12741 | NM_175850 | 1789 | ENSG00000088305 | DNMT3B |
| chr20 | 31381300 | Kanga11a\|DNA\|TcMar-Tc2 | 13657 | NM_175850 | 1789 | ENSG00000088305 | DNMT3B |
| chr20 | 31386255 | exon (NM_175848, exon 14 of 22) | 18612 | NM_175850 | 1789 | ENSG00000088305 | DNMT3B |
| chr20 | 31388050 | exon (NM_175848, exon 16 of 22) | −19695 | NM_012325 | 22919 | ENSG00000101367 | MAPRE1 |
| chr20 | 31389120 | exon (NM_175848, exon 18 of 22) | −18625 | NM_012325 | 22919 | ENSG00000101367 | MAPRE1 |
| chr20 | 31393185 | exon (NM_175848, exon 20 of 22) | −14560 | NM_012325 | 22919 | ENSG00000101367 | MAPRE1 |
| chr20 | 31395662 | exon (NM_175848, exon 22 of 22) | −12083 | NM_012325 | 22919 | ENSG00000101367 | MAPRE1 |
| chrX | 66121000 | Intergenic | −262860 | NM_001199687 | 60401 | ENSG00000131080 | EDA2R |
| chrX | 66766332 | exon (NM_001348064, exon 1 of 3) | 1882 | NM_001011645 | 367 | ENSG00000169083 | AR |
| chrX | 66931448 | exon (NM_000044, exon 4 of 8) | 166998 | NM_001011645 | 367 | ENSG00000169083 | AR |
| chrX | 66937357 | exon (NM_000044exon 5 of 8) | 172907 | NM_001011645 | 367 | ENSG00000169083 | AR |
| chrX | 66943528 | exon (NM_000044, exon 8 of 8) | 179082 | NM_001011645 | 367 | ENSG00000169083 | AR |
| chr2 | 25452399 | non-coding (NR_135490, exon 24 of 24) | 22596 | NM_001320893 | 1788 | ENSG00000119772 | DNMT3A |
| chr2 | 25452969 | non-coding (NR_135490, exon 24 of 24) | 22026 | NM_001320893 | 1788 | ENSG00000119772 | DNMT3A |
| chr2 | 25453099 | non-coding (NR_135490, exon 24 of 24) | 21896 | NM_001320893 | 1788 | ENSG00000119772 | DNMT3A |
| chr2 | 25453105 | non-coding (NR_135490, exon 24 of 24) | 21890 | NM_001320893 | 1788 | ENSG00000119772 | DNMT3A |
| chr2 | 25453799 | non-coding (NR_135490, exon 24 of 24) | 21196 | NM_001320893 | 1788 | ENSG00000119772 | DNMT3A |
| chr2 | 25453898 | non-coding (NR_135490, exon 24 of 24) | 21097 | NM_001320893 | 1788 | ENSG00000119772 | DNMT3A |
| chr2 | 25453899 | non-coding (NR_135490, exon 24 of 24) | 21096 | NM_001320893 | 1788 | ENSG00000119772 | DNMT3A |
| chr2 | 25453913 | non-coding (NR_135490, exon 24 of 24) | 21082 | NM_001320893 | 1788 | ENSG00000119772 | DNMT3A |
| chr2 | 25499982 | CpG | −24803 | NM_153759 | 1788 | ENSG00000119772 | DNMT3A |
| chr2 | 25500271 | CpG | −25092 | NM_153759 | 1788 | ENSG00000119772 | DNMT3A |
| chr2 | 25500272 | CpG | −25093 | NM_153759 | 1788 | ENSG00000119772 | DNMT3A |
| chr2 | 239147428 | 3′ UTR (NM_018645, exon 4 of 4) | 1246 | NM_018645 | 55502 | ENSG00000144485 | HES6 |
| chr2 | 239147440 | 3′ UTR (NM_018645, exon 4 of 4) | 1234 | NM_018645 | 55502 | ENSG00000144485 | HES6 |
| chr2 | 239147441 | 3′ UTR (NM_018645, exon 4 of 4) | 1233 | NM_018645 | 55502 | ENSG00000144485 | HES6 |
| chr2 | 239147485 | exon (NM_018645, exon 4 of 4) | 1189 | NM_018645 | 55502 | ENSG00000144485 | HES6 |
| chr2 | 239147486 | exon (NM_018645, exon 4 of 4) | 1188 | NM_018645 | 55502 | ENSG00000144485 | HES6 |
| chr2 | 239147522 | exon (NM_018645, exon 4 of 4) | 1152 | NM_018645 | 55502 | ENSG00000144485 | HES6 |
| chr2 | 239147534 | exon (NM_018645, exon 4 of 4) | 1140 | NM_018645 | 55502 | ENSG00000144485 | HES6 |
| chr2 | 239147589 | exon (NM_018645, exon 4 of 4) | 1085 | NM_018645 | 55502 | ENSG00000144485 | HES6 |
| chr2 | 239147601 | exon (NM_018645, exon 4 of 4) | 1073 | NM_018645 | 55502 | ENSG00000144485 | HES6 |
| chr2 | 239147614 | exon (NM_018645, exon 4 of 4) | 1060 | NM_018645 | 55502 | ENSG00000144485 | HES6 |
| chr2 | 239147637 | exon (NM_018645, exon 4 of 4) | 1037 | NM_018645 | 55502 | ENSG00000144485 | HES6 |
| chr2 | 239147649 | exon (NM_018645, exon 4 of 4) | 1025 | NM_018645 | 55502 | ENSG00000144485 | HES6 |
| chr2 | 239147665 | exon (NM_018645, exon 4 of 4) | 1009 | NM_018645 | 55502 | ENSG00000144485 | HES6 |
| chr2 | 239149894 | CpG-16151 | −1220 | NM_001142853 | 55502 | ENSG00000144485 | HES6 |
| chr2 | 239149895 | CpG-16151 | −1221 | NM_001142853 | 55502 | ENSG00000144485 | HES6 |
| chr3 | 12838468 | CpG | 294 | NM_012298 | 23066 | ENSG00000144712 | CAND2 |
| chr3 | 50660608 | intron (NM_001243925, intron 2 of 10) | 6031 | NM_001243925 | 7867 | ENSG00000114738 | MAPKAPK3 |
| chr3 | 50670155 | intron (NM_001243925, intron 2 of 10) | 15578 | NM_001243925 | 7867 | ENSG00000114738 | MAPKAPK3 |
| chr3 | 128202781 | exon (NM_032638, exon 4 of 6) | 3978 | NM_001145662 | 2624 | ENSG00000179348 | GATA2 |
| chr3 | 128202787 | exon (NM_032638, exon 4 of 6) | 3972 | NM_001145662 | 2624 | ENSG00000179348 | GATA2 |
| chr3 | 128202792 | exon (NM_032638, exon 4 of 6) | 3967 | NM_001145662 | 2624 | ENSG00000179348 | GATA2 |
| chr3 | 128202795 | exon (NM_032638, exon 4 of 6) | 3964 | NM_001145662 | 2624 | ENSG00000179348 | GATA2 |
| chr3 | 128202796 | exon (NM_032638, exon 4 of 6) | 3963 | NM_001145662 | 2624 | ENSG00000179348 | GATA2 |
| chr3 | 128202884 | intron (NM_001145661, intron 4 of 6) | 3875 | NM_001145662 | 2624 | ENSG00000179348 | GATA2 |
| chr3 | 128202894 | intron (NM_001145661, intron 4 of 6) | 3865 | NM_001145662 | 2624 | ENSG00000179348 | GATA2 |
| chr3 | 128202895 | intron (NM_001145661, intron 4 of 6) | 3864 | NM_001145662 | 2624 | ENSG00000179348 | GATA2 |
| chr3 | 128202927 | intron (NM_001145661, intron 4 of 6) | 3832 | NM_001145662 | 2624 | ENSG00000179348 | GATA2 |
| chr3 | 128202928 | intron (NM_001145661, intron 4 of 6) | 3831 | NM_001145662 | 2624 | ENSG00000179348 | GATA2 |
| chr3 | 128205157 | exon (NM_032638, exon 3 of 6) | 1602 | NM_001145662 | 2624 | ENSG00000179348 | GATA2 |
| chr3 | 128205179 | exon (NM_032638, exon 3 of 6) | 1580 | NM_001145662 | 2624 | ENSG00000179348 | GATA2 |
| chr3 | 128205180 | exon (NM_032638, exon 3 of 6) | 1579 | NM_001145662 | 2624 | ENSG00000179348 | GATA2 |
| chr3 | 128205195 | exon (NM_032638, exon 3 of 6) | 1564 | NM_001145662 | 2624 | ENSG00000179348 | GATA2 |
| chr3 | 128205196 | exon (NM_032638, exon 3 of 6) | 1563 | NM_001145662 | 2624 | ENSG00000179348 | GATA2 |
| chr3 | 128205210 | intron (NM_001145661, intron 3 of 6) | 1549 | NM_001145662 | 2624 | ENSG00000179348 | GATA2 |
| chr3 | 128210723 | CpG-18893 | 1302 | NM_032638 | 2624 | ENSG00000179348 | GATA2 |
| chr3 | 128210727 | CpG-18893 | 1298 | NM_032638 | 2624 | ENSG00000179348 | GATA2 |
| chr3 | 128212207 | promoter-TSS (NM_032638) | −182 | NM_032638 | 2624 | ENSG00000179348 | GATA2 |
| chr3 | 185817004 | intron (NM_004454, intron 5 of 12) | 9869 | NM_004454 | 2119 | ENSG00000244405 | ETV5 |
| chr4 | 16084875 | promoter-TSS (NM_001371407) | 79 | NM_001371406 | | | |
| chr4 | 16084881 | promoter-TSS (NM_001371407) | 73 | NM_001371406 | | | |
| chr4 | 16084898 | promoter-TSS (NM_001371407) | 56 | NM_001371406 | | | |
| chr4 | 16085260 | promoter-TSS (NM_001145852) | 87 | NM_001145847 | 8842 | ENSG00000007062 | PROM1 |
| chr4 | 16085380 | promoter-TSS (NM_001145852) | −33 | NM_00145852 | 8842 | ENSG00000007062 | PROM1 |
| chr4 | 16085396 | promoter-TSS (NM_001145852) | −49 | NM_001145852 | 8842 | ENSG00000007062 | PROM1 |
| chr4 | 16085398 | promoter-TSS (NM_001145852) | −51 | NM_001145852 | 8842 | ENSG00000007062 | PROM1 |
| chr4 | 16085401 | promoter-TSS (NM_001145852) | −54 | NM_001145852 | 8842 | ENSG00000007062 | PROM1 |
| chr5 | 172661872 | exon (NM_004387, exon 1 of 2) | 332 | NM_001166175 | 1482 | ENSG00000183072 | NKX2-5 |
| chr5 | 172661874 | exon (NM_004387, exon 1 of 2) | 330 | NM_001166175 | 1482 | ENSG00000183072 | NKX2-5 |
| chr6 | 34522044 | intron (NM_012391, intron 1 of 5) | 2057 | NM_012391 | 25803 | ENSG00000124664 | SPDEF |
| chr6 | 34523223 | intron (NM_012391, intron 1 of 5) | 878 | NM_012391 | 25803 | ENSG00000124664 | SPDEF |
| chr8 | 11561726 | promoter-TSS (NM_002052) | 20 | NM_002052 | 2626 | ENSG00000136574 | GATA4 |
| chr8 | 11565855 | exon (NM_002052, exon 2 of 7) | 495 | NM_001308093 | 2626 | ENSG00000136574 | GATA4 |
| chr8 | 11566229 | exon (NM_002052, exon 2 of 7) | 869 | NM_001308093 | 2626 | ENSG00000136574 | GATA4 |
| chr8 | 11583613 | intron (NM_002052, intron 2 of 6) | 18253 | NM_001308093 | 2626 | ENSG00000136574 | GATA4 |
| chr8 | 11583614 | intron (NM_002052, intron 2 of 6) | 18254 | NM_001308093 | 2626 | ENSG00000136574 | GATA4 |
| chr9 | 126775063 | CpG | 1021 | NM_004789 | 9355 | ENSG00000106689 | LHX2 |
| chr9 | 126775082 | CpG | 1040 | NM_004789 | 9355 | ENSG00000106689 | LHX2 |
| chr9 | 126776013 | CpG | 1971 | NM_004789 | 9355 | ENSG00000106689 | LHX2 |
| chr9 | 126776231 | CpG | 2189 | NM_004789 | 9355 | ENSG00000106689 | LHX2 |
| chr9 | 139437635 | intron (NM_017617, intron 2 of 33) | 2860 | NM_017617 | 4851 | ENSG00000148400 | NOTCH1 |
| chr9 | 139437679 | intron (NM_017617, intron 2 of 33) | 2816 | NM_017617 | 4851 | ENSG00000148400 | NOTCH1 |
| chr10 | 23481414 | 5′ UTR (NM_178161, exon 1 of 2) | 178 | NM_178161 | 256297 | ENSG00000168267 | PTF1A |
| chr10 | 23481415 | 5′ UTR (NM_178161, exon 1 of 2) | 179 | NM_178161 | 256297 | ENSG00000108267 | PTF1A |
| chr10 | 23481417 | 5′ UTR (NM_178161, exon 1 of 2) | 181 | NM_178161 | 256297 | ENSG00000168267 | PTF1A |
| chr10 | 23481445 | 5′ UTR (NM_178161, exon 1 of 2) | 209 | NM_178161 | 256297 | ENSG00000168267 | PTF1A |
| chr10 | 23481447 | 5′ UTR (NM_178161, exon 1 of 2) | 211 | NM_178161 | 256297 | ENSG00000168267 | PTF1A |
| chr10 | 23481448 | 5′ UTR (NM_178161, exon 1 of 2) | 212 | NM_178161 | 256297 | ENSG00000168267 | PTF1A |
| chr10 | 23481451 | 5′ UTR (NM_178161, exon 1 of 2) | 215 | NM_178161 | 256297 | ENSG00000168267 | PTF1A |
| chr10 | 23481461 | exon (NM_178161, exon 1 of 2) | 225 | NM_178161 | 256297 | ENSG00000168267 | PTF1A |
| chr10 | 23481462 | exon (NM_78161, exon 1 of 2) | 226 | NM_178161 | 256297 | ENSG00000168267 | PTF1A |
| chr10 | 23481463 | exon (NM_178161, exon 1 of 2) | 227 | NM_178161 | 256297 | ENSG00000168267 | PTF1A |
| chr10 | 56699066 | AluY\|SINE\|Alu | −138020 | NM_001142767 | 65217 | ENSG00000150275 | PCDH15 |
| chr10 | 56699077 | AluY\|SINE\|Alu | −138031 | NM_001142767 | 65217 | ENSG00000150275 | PCDH15 |
| chr10 | 57391015 | CpG | −3318 | NM_001354404 | 65217 | ENSG00000150275 | PCDH15 |
| chr11 | 69451450 | CpG | −4418 | NM_053056 | 595 | ENSG00000110092 | CCND1 |
| chr12 | 103344993 | intron (NM_001354304, intron 1 of 13) | −6454 | NM_004316 | 429 | ENSG00000139352 | ASCL1 |
| chr12 | 103344998 | intron (NM_001354304, intron 1 of 13) | −6449 | NM_004316 | 429 | ENSG00000139352 | ASCL1 |
| chr12 | 103345006 | intron (NM_001354304, intron 1 of 13) | −6441 | NM_004316 | 429 | ENSG00000139352 | ASCL1 |
| chr12 | 103345029 | intron (NM_001354304, intron 1 of 13) | −6418 | NM_004316 | 429 | ENSG00000139352 | ASCL1 |
| chr12 | 103345033 | intron (NM_001354304, intron 1 of 13) | −6414 | NM_004316 | 429 | ENSG00000139352 | ASCL1 |
| chr14 | 51560040 | intron (NM_052978, intron 1 of 6) | 2377 | NM_015163 | 114088 | ENSG00000100505 | TRIM9 |
| chr14 | 51560162 | CpG | 2255 | NM_015163 | 114088 | ENSG00000100505 | TRIM9 |
| chr14 | 51560191 | CpG | 2226 | NM_015163 | 114088 | ENSG00000100505 | TRIM9 |
| chr14 | 51560193 | CpG | 2224 | NM_015163 | 114088 | ENSG00000100505 | TRIM9 |
| chr14 | 51560208 | CpG | 2209 | NM_015163 | 114088 | ENSG00000100505 | TRIM9 |
| chr14 | 51560259 | CpG | 2158 | NM_015163 | 114088 | ENSG00000100505 | TRIM9 |
| chr14 | 51560261 | CpG | 2156 | NM_015163 | 114088 | ENSG00000100505 | TRIM9 |
| chr14 | 51560275 | CpG | 2142 | NM_015163 | 114088 | ENSG00000100505 | TRIM9 |
| chr14 | 65409361 | promoter-TSS (NM_002083) | 97 | NR_046321 | 2877 | ENSG00000176153 | GPX2 |
| chr14 | 93385930 | Intergenic | −3560 | NM_001301690 | 1113 | ENSG00000100604 | CHGA |
| chr14 | 93423499 | intron (NM_001142593, intron 8 of 10) | 34009 | NM_001275 | 1113 | ENSG00000100604 | CHGA |
| chr14 | 93423555 | intron (NM_001142593, intron 8 of 10) | 34065 | NM_001275 | 1113 | ENSG00000100604 | CHGA |
| chr14 | 93425158 | intron (NM_001142593, intron 7 of 10) | 35668 | NM_001275 | 1113 | ENSG00000100604 | CHGA |
| chr14 | 93425189 | MIRb\|SINE\|MIR | 35699 | NM_001275 | 1113 | ENSG00000100604 | CHGA |
| chr14 | 93425207 | MIRb\|SINE\|MIR | 35717 | NM_001275 | 1113 | ENSG00000100604 | CHGA |
| chr14 | 93425289 | MIRb\|SINE\|MIR | 35799 | NM_001275 | 1113 | ENSG00000100604 | CHGA |
| chr14 | 93425328 | MIRb\|SINE\|MIR | 35838 | NM_001275 | 1113 | ENSG00000100604 | CHGA |
| chr14 | 93425517 | intron (NM_001142593, intron 7 of 10) | 36027 | NM_001275 | 1113 | ENSG00000100604 | CHGA |
| chr14 | 93425684 | intron (NM_001142593, intron 7 of 10) | 36194 | NM_001275 | 1113 | ENSG00000100604 | CHGA |
| chr14 | 93425685 | intron (NM_001142593, intron 7 of 10) | 36195 | NM_001275 | 1113 | ENSG00000100604 | CHGA |
| chr14 | 93425738 | intron (NM_001142593, intron 7 of 10) | 36248 | NM_001275 | 1113 | ENSG00000100604 | CHGA |
| chr14 | 93426226 | intron (NM_001142593, intron 7 of 10) | 36736 | NM_001275 | 1113 | ENSG00000100604 | CHGA |
| chr14 | 93426244 | intron (NM_001142593, intron 7 of 10) | 36754 | NM_001275 | 1113 | ENSG00000100604 | CHGA |
| chr14 | 93426333 | intron (NM_001142593, intron 7 of 10) | 36843 | NM_001275 | 1113 | ENSG00000100604 | CHGA |
| chr14 | 93426378 | intron (NM_001142593, intron 7 of 10) | 36888 | NM_001275 | 1113 | ENSG00000100604 | CHGA |
| chr14 | 93426872 | MIRb\|SINE\|MIR | 37382 | NM_001275 | 1113 | ENSG00000100604 | CHGA |
| chr14 | 93426913 | MIRb\|SINE\|MIR | 37423 | NM_001275 | 1113 | ENSG00000100604 | CHGA |
| chr14 | 93426914 | MIRb\|SINE\|MIR | 37424 | NM_001275 | 1113 | ENSG00000100604 | CHGA |
| chr14 | 93426959 | intron (NM_001142593, intron 7 of 10) | 37469 | NM_001275 | 1113 | ENSG00000100604 | CHGA |
| chr14 | 93439910 | intron (NM_001142593, intron 5 of 10) | 50420 | NM_001275 | 1113 | ENSG00000100604 | CHGA |
| chr18 | 31323189 | exon (NM_030632, exon 12 of 12) | 165011 | NM_030632 | 80816 | ENSG00000141431 | ASXL3 |
| chr18 | 31323202 | exon (NM_030632, exon 12 of 12) | 165024 | NM_030632 | 80816 | ENSG00000141431 | ASXL3 |
| chr18 | 53175324 | intron (NM_001369572, intron 3 of 19) | 2674 | NM_001348211 | 6925 | ENSG00000196628 | TCF4 |
| chr18 | 53305364 | AluJb\|SINE\|Alu | −2181 | NM_001243226 | 6925 | ENSG00000196628 | TCF4 |
| chr18 | 53305490 | AluJb\|SINE\|Alu | −2307 | NM_001243226 | 6925 | ENSG00000196628 | TCF4 |
| chr19 | 11086495 | intron (NM_003072, intron 1 of 34) | −8328 | NM_001128846 | 6597 | ENSG00000127616 | SMARCA4 |
| chr19 | 39993532 | exon (NM_016941, exon 4 of 8) | 3950 | NM_203486 | 10683 | ENSG00000090932 | DLL3 |
| chr19 | 39993551 | exon (NM_016941, exon 4 of 8) | 3969 | NM_203486 | 10683 | ENSG00000090932 | DLL3 |
| chr19 | 39993567 | exon (NM_016941, exon 4 of 8) | 3985 | NM_203486 | 10683 | ENSG00000090932 | DLL3 |
| chr19 | 39993606 | exon (NM_016941, exon 4 of 8) | 4024 | NM_203486 | 10683 | ENSG00000090932 | DLL3 |
| chr19 | 42618585 | L2c\|LINE\|L2 | 18008 | NM_002698 | 5452 | ENSG00000028277 | POU2F2 |
| chr19 | 42618760 | intron (NM_002698, intron 5 of 13) | 17833 | NM_002698 | 5452 | ENSG00000028277 | POU2F2 |
| chr19 | 42618787 | MIR3\|SINE\|MIR | 17806 | NM_002698 | 5452 | ENSG00000028277 | POU2F2 |
| chr19 | 42618789 | MIR3\|SINE\|MIR | 17804 | NM_002698 | 5452 | ENSG00000028277 | POU2F2 |
| chr19 | 42618823 | MIR3\|SINE\|MIR | 17770 | NM_002698 | 5452 | ENSG00000028277 | POU2F2 |
| chr20 | 20232309 | exon (NM_015585, exon 20 of 27) | −116434 | NM_002196 | 3642 | ENSG00000173404 | INSM1 |
| chr20 | 20232319 | exon (NM_015585, exon 20 of 27) | −116424 | NM_002196 | 3642 | ENSG00000173404 | INSM1 |
| chr20 | 20232333 | exon (NM_015585, exon 20 of 27) | −116410 | NM_002196 | 3642 | ENSG00000173404 | INSM1 |
| chr20 | 20232347 | exon (NM_015585, exon 20 of 27) | −116396 | NM_002196 | 3642 | ENSG00000173404 | INSM1 |
| chr20 | 20232348 | exon (NM_015585, exon 20 of 27) | −116395 | NM_002196 | 3642 | ENSG00000173404 | INSM1 |
| chr20 | 20232351 | exon (NM_015585, exon 20 of 27) | −116392 | NM_002196 | 3642 | ENSG00000173404 | INSM1 |
| chr20 | 20232352 | exon (NM_015585, exon 20 of 27) | −116391 | NM_002196 | 3642 | ENSG00000173404 | INSM1 |
| chr20 | 20232357 | exon (NM_015585, exon 20 of 27) | −116386 | NM_002196 | 3642 | ENSG00000173404 | INSM1 |
| chr20 | 20232358 | exon (NM_015585, exon 20 of 27) | −116385 | NM_002196 | 3642 | ENSG00000173404 | INSM1 |
| chr20 | 20232367 | exon (NM_015585, exon 20 of 27) | −116376 | NM_002196 | 3642 | ENSG00000173404 | INSM1 |
| chr20 | 20232388 | exon (NM_015585, exon 20 of 27) | −116355 | NM_002196 | 3642 | ENSG00000173404 | INSM1 |
| chr20 | 20266415 | AluJb\|SINE\|Alu | −82328 | NM_002196 | 3642 | ENSG00000173404 | INSM1 |
| chr20 | 20376954 | intron (NM_020343, intron 39 of 39) | 28211 | NM_002196 | 3642 | ENSG00000173404 | INSM1 |
| chr20 | 20376969 | intron (NM_020343, intron 39 of 39) | 28226 | NM_002196 | 3642 | ENSG00000173404 | INSM1 |
| chr20 | 20377086 | intron (NM_020343, intron 39 of 39) | 28343 | NM_002196 | 3642 | ENSG00000173404 | INSM1 |
| chr20 | 20377158 | intron (NM_020343, intron 39 of 39) | 28415 | NM_002196 | 3642 | ENSG00000173404 | INSM1 |
| chr20 | 20377165 | intron (NM_020343, intron 39 of 39) | 28422 | NM_002196 | 3642 | ENSG00000173404 | INSM1 |
| chr20 | 20421292 | intron (NM_020343, intron 37 of 39) | 72549 | NM_002196 | 3642 | ENSG00000173404 | INSM1 |
| chr20 | 20421311 | intron (NM_020343, intron 37 of 39) | 72568 | NM_002196 | 3642 | ENSG00000173404 | INSM1 |
| chr20 | 20421456 | intron (NM_020343, intron 37 of 39) | 72713 | NM_002196 | 3642 | ENSG00000173404 | INSM1 |
| chr20 | 20421483 | intron (NM_020343, intron 37 of 39) | 72740 | NM_002196 | 3642 | ENSG00000173404 | INSM1 |
| chr20 | 20433160 | CpG | 84417 | NM_002196 | 3642 | ENSG00000173404 | INSM1 |
| chr20 | 20433163 | CpG | 84420 | NM_002196 | 3642 | ENSG00000173404 | INSM1 |
| chr20 | 20433166 | CpG | 84423 | NM_002196 | 3642 | ENSG00000173404 | INSM1 |
| chr20 | 20433198 | CpG | 84455 | NM_002196 | 3642 | ENSG00000173404 | INSM1 |
| chr20 | 20433208 | CpG | 84465 | NM_002196 | 3642 | ENSG00000173404 | INSM1 |
| chr20 | 20433248 | CpG | 84505 | NM_002196 | 3642 | ENSG00000173404 | INSM1 |
| chr20 | 20433256 | CpG | 84513 | NM_002196 | 3642 | ENSG00000173404 | INSM1 |
| chr20 | 20433263 | CpG | 84520 | NM_002196 | 3642 | ENSG00000173404 | INSM1 |
| chr20 | 20433284 | CpG | 84541 | NM_002196 | 3642 | ENSG00000173404 | INSM1 |
| chr20 | 20433293 | CpG | 84550 | NM_002196 | 3642 | ENSG00000173404 | INSM1 |
| chr20 | 21490704 | TTS (NM_002509) | 3993 | NM_002509 | 4821 | ENSG00000125820 | NKX2-2 |
| chr20 | 21490799 | TTS (NM_002509) | 3898 | NM_002509 | 4821 | ENSG00000125820 | NKX2-2 |
| chr20 | 21490807 | TTS (NM_002509) | 3890 | NM_002509 | 4821 | ENSG00000125820 | NKX2-2 |
| chr20 | 21494139 | exon (NM_002509, exon 1 of 2) | 558 | NM_002509 | 4821 | ENSG00000125820 | NKX2-2 |
| chr20 | 21494141 | exon (NM_002509, exon 1 of 2) | 556 | NM_002509 | 4821 | ENSG00000125820 | NKX2-2 |
| chr20 | 21494237 | exon (NM_002509, exon 1 of 2) | 460 | NM_002509 | 4821 | ENSG00000125820 | NKX2-2 |
| chr21 | 22564429 | AluSq2\|SINE\|Alu | 44954 | NM_001352597 | 4685 | ENSG00000154654 | NCAM2 |
| chr21 | 42850711 | MER81\|DNA\|hAT-Blackjack | 29276 | NM_001135099 | 7113 | ENSG00000184012 | TMPRSS2 |
| chr21 | 42860322 | exon (NM_001135099, exon 5 of 14) | 19665 | NM_001135099 | 7113 | ENSG00000184012 | TMPRSS2 |
| chr21 | 42860341 | exon (NM_001135099, exon 5 of 14) | 19646 | NM_001135099 | 7113 | ENSG00000184012 | TMPRSS2 |
| chr21 | 42880610 | promoter-TSS (NM_001135099) | −530 | NM_005656 | 7113 | ENSG00000184012 | TMPRSS2 |
| chr21 | 42880614 | promoter-TSS (NM_001135099) | −534 | NM_005656 | 7113 | ENSG00000184012 | TMPRSS2 |
| chr21 | 42880659 | promoter-TSS (NM_001135099) | −579 | NM_005656 | 7113 | ENSG00000184012 | TMPRSS2 |
| chr21 | 42880668 | promoter-TSS (NM_001135099) | −588 | NM_005656 | 7113 | ENSG00000184012 | TMPRSS2 |
| chr1 | 3052478 | intron (NM_199454, intron 1 of 16) | 7944 | NR_036215 | 100422968 | ENSG00000283572 | MIR4251 |
| chr1 | 26735081 | Intergenic | −9999 | NM_024674 | 79727 | ENSG00000131914 | LIN28A |
| chr1 | 26735241 | Intergenic | −2062 | NM_024674 | 79727 | ENSG00000131914 | LIN28A |
| chr1 | 26735243 | Intergenic | −2060 | NM_024674 | 79727 | ENSG00000131914 | LIN28A |
| chr1 | 26735251 | Intergenic | −2052 | NM_024674 | 79727 | ENSG00000131914 | LIN28A |
| chr1 | 65721361 | L2a\|LINE\|L2 | −9011 | NM_001256865 | 9829 | ENSG00000116675 | DNAJC6 |
| chr1 | 229195756 | Intergenic | 114787 | NR_039659 | 100616234 | ENSG00000283468 | MIR4454 |
| chr2 | 23807028 | intron (NM_052920, intron 3 of 13) | 198983 | NM_052920 | 114818 | ENSG00000119771 | KLHL29 |
| chr2 | 171679652 | CpG-15697 | 6260 | NM_000817 | 2571 | ENSG00000128683 | GAD1 |
| chr2 | 236877206 | exon (NM_001037131, exon 13 of 18) | 199818 | NM_001301687 | 2637 | ENSG00000168505 | GBX2 |
| chr2 | 242549633 | CpG | 7278 | NM_001164356 | 51078 | ENSG00000176946 | THAP4 |
| chr2 | 242549635 | CpG | 7276 | NM_001164356 | 51078 | ENSG00000176946 | THAP4 |
| chr2 | 242549638 | CpG | 7273 | NM_001164356 | 51078 | ENSG00000176946 | THAP4 |
| chr2 | 242549659 | CpG | 7252 | NM_001164356 | 51078 | ENSG00000176946 | THAP4 |
| chr3 | 26664523 | promoter-TSS (NM_001317808) | 231 | NM_052953 | 116135 | ENSG00000179796 | LRRC3B |
| chr3 | 126664525 | promoter-TSS (NM_001317808) | 233 | NM_052953 | 116135 | ENSG00000179796 | LRRC3B |
| chr3 | 111431556 | intron (NM_001185106, intron 2 of 4) | −19782 | NM_001134437 | 90102 | ENSG00000144824 | PHLDB2 |
| chr4 | 169799044 | exon (NM_001166110, exon 2 of 12) | 45803 | NM_001367570 | 23022 | ENSG00000129116 | PALED |
| chr5 | 138441347 | intron (NM_001037633, intron 4 of 10) | 92705 | NM_022464 | 64374 | ENSG00000120725 | SIL1 |
| chr5 | 138441374 | intron (NM_001037633, intron 4 of 10) | 92678 | NM_022464 | 64374 | ENSG00000120725 | SIL1 |
| chr5 | 138441376 | intron (NM_001037633, intron 4 of 10) | 92676 | NM_022464 | 64374 | ENSG00000120725 | SIL1 |
| chr5 | 176038510 | Intergenic | −1358 | NM_052899 | 114787 | ENSG00000169258 | GPRIN1 |
| chr5 | 176038524 | Intergenic | −1372 | NM_052899 | 114787 | ENSG00000169258 | GPRIN1 |
| chr5 | 176038525 | Intergenic | −1373 | NM_052899 | 114787 | ENSG00000169258 | GPRIN1 |
| chr5 | 176038562 | Intergenic | −1410 | NM_052899 | 114787 | ENSG00000169258 | GPRIN1 |
| chr5 | 179401799 | intron (NM_018434, intron 5 of 8) | 40593 | NR_029885 | 442908 | ENSG00000198995 | MIR340 |
| chr6 | 15265454 | intron (NM_001267040, intron 1 of 17) | 16373 | NM_001267040 | 3720 | ENSG00000008083 | JARID2 |
| chr6 | 168462402 | intron (NR_110312, intron 12 of 15) | 14108 | NM_001122841 | 79981 | ENSG00000153303 | FRMD1 |
| chr6 | 168462447 | intron (NR_110312, intron 12 of 15) | 14063 | NM_001122841 | 79981 | ENSG00000153303 | FRMD1 |
| chr6 | 170578588 | intron (NR_126021, intron 2 of 2) | −2969 | NR_104177 | 401289 | | LINC01624 |
| chr7 | 2968311 | exon (NM_032415, exon 13 of 25) | −84374 | NM_007353 | 2768 | ENSG00000146535 | GNA12 |
| chr7 | 2968441 | CpG | −84504 | NM_007353 | 2768 | ENSG00000146535 | GNA12 |
| chr7 | 2968442 | CpG | −84505 | NM_007353 | 2768 | ENSG00000146535 | GNA12 |
| chr7 | 2968451 | CpG | −84514 | NM_007353 | 2768 | ENSG00000146535 | GNA12 |
| chr7 | 2968454 | CpG | −84517 | NM_007353 | 2768 | ENSG00000146535 | GNA12 |
| chr7 | 5572006 | Intergenic | −1779 | NM_001101 | 60 | ENSG00000075624 | ACTB |
| chr7 | 150812220 | exon (NM_001308305, exon 1 of 9) | 500 | NM_001308305 | 116988 | ENSG00000133612 | AGAP3 |
| chr8 | 47570600 | L1M2\|LINE\|L1 | −41340 | NR_146077 | 389652 | ENSG00000248498 | ASNSP1 |
| chr9 | 116411412 | Intergenic | 55008 | NM_001276262 | 5998 | ENSG00000138835 | RGS3 |
| chr9 | 124498726 | CpG | −6333 | NM_138709 | 153090 | ENSG00000136848 | DAB2IP |
| chr9 | 124620057 | intron (NM_001139442, intron 8 of 8) | 114998 | NM_138709 | 153090 | ENSG00000136848 | DAB2IP |
| chr10 | 676777 | non-coding (NR_147612, exon 2 of 2) | 2278 | NR_147612 | 101930421 | ENSG00000225140 | LOC101930421 |
| chr10 | 676811 | non-coding (NR_147612, exon 2 of 2) | 2312 | NR_147612 | 101930421 | ENSG00000225140 | LOC101930421 |
| chr11 | 2000638 | Intergenic | 10507 | NR_024471 | 100133545 | ENSG00000226416 | MRPL23-AS1 |
| chr11 | 11600192 | intron (NM_001363464, intron 1 of 9) | 43355 | NM_001363464 | 374378 | ENSG00000110328 | GALNT18 |
| chr11 | 11600210 | intron (NM_001363464, intron 1 of 9) | 43337 | NM_001363464 | 374378 | ENSG00000110328 | GALNT18 |
| chr11 | 11600228 | intron (NM_001363464, intron 1 of 9) | 43319 | NM_001363464 | 374378 | ENSG00000110328 | GALNT18 |
| chr11 | 11600230 | intron (NM_001363464, intron 1 of 9) | 43317 | NM_001363464 | 374378 | ENSG00000110328 | GALNT18 |
| chr11 | 11600231 | intron (NM_001363464, intron 1 of 9) | 43316 | NM_001363464 | 374378 | ENSG00000110328 | GALNT18 |
| chr11 | 11600240 | intron (NM_001363464, intron 1 of 9) | 43307 | NM_001363464 | 374378 | ENSG00000110328 | GALNT18 |
| chr11 | 64120601 | exon (NM_032251, exon 21 of 27) | −6028 | NM_001318361 | 8986 | ENSG00000162302 | RPS6KA4 |
| chr11 | 70851727 | intron (NM_012309, intron 2 of 22) | 84110 | NM_012309 | 22941 | ENSG00000162105 | SHANK2 |
| chr12 | 40618451 | promoter-TSS (NM_198578) | −343 | NM_198578 | 120892 | ENSG00000188906 | LRRK2 |
| chr12 | 40618493 | promoter-TSS (NM_198578) | −301 | NM_198578 | 120892 | ENSG00000188906 | LRRK2 |
| chr15 | 32385689 | intron (NM_000746, intron 2 of 9) | 62976 | NM_000746 | 1139 | ENSG00000175344 | CHRNA7 |
| chr15 | 68723747 | CpG | 750 | NM_001004439 | 22801 | ENSG00000137809 | ITGA11 |
| chr15 | 89954601 | CpG | 11204 | NR_160724 | 105371031 | | LOC105371031 |
| chr15 | 89954603 | CpG | 11206 | NR_160724 | 105371031 | | LOC105371031 |
| chr15 | 89954605 | CpG | 11208 | NR_160724 | 105371031 | | LOC105371031 |
| chr15 | 89959942 | CpG | 16545 | NR_160724 | 105371031 | | LOC105371031 |
| chr16 | 85498200 | MIRb\|SINE\|MIR | −146810 | NM_001134473 | 23199 | ENSG00000131149 | GSE1 |
| chr16 | 85498219 | Intergenic | −146791 | NM_001134473 | 23199 | ENSG00000131149 | GSE1 |
| chr16 | 85498220 | Intergenic | −146790 | NM_001134473 | 23199 | ENSG00000131149 | GSE1 |
| chr16 | 85498227 | Intergenic | −146783 | NM_001134473 | 23199 | ENSG00000131149 | GSE1 |
| chr16 | 85498781 | Intergenic | −146229 | NM_001134473 | 23199 | ENSG00000131149 | GSE1 |
| chr16 | 85519155 | CpG | −125855 | NM_001134473 | 23199 | ENSG00000131149 | GSE1 |
| chr16 | 85519162 | CpG | −125848 | NM_001134473 | 23199 | ENSG00000131149 | GSE1 |
| chr16 | 85519165 | CpG | −125845 | NM_001134473 | 23199 | ENSG00000131149 | GSE1 |
| chr16 | 85519168 | CpG | −125842 | NM_001134473 | 23199 | ENSG00000131149 | GSE1 |
| chr16 | 85519171 | CpG | −125839 | NM_001134473 | 23199 | ENSG00000131149 | GSE1 |
| chr16 | 85519173 | CpG | −125837 | NM_001134473 | 23199 | ENSG00000131149 | GSE1 |
| chr16 | 85519303 | CpG | −125707 | NM_001134473 | 23199 | ENSG00000131149 | GSE1 |
| chr16 | 88715953 | intron (NM_000101, intron 1 of 5) | 1503 | NM_000101 | 1535 | ENSG00000051523 | CYBA |
| chr17 | 28299568 | intron (NR_026738, intron 4 of 15) | 30921 | NM_198529 | 374786 | ENSG00000176927 | EFCAB5 |
| chr17 | 80372745 | intron (NR_033265, intron 2 of 9) | −3473 | NM_001369488 | 284004 | ENSG00000169660 | HEXD |
| chr17 | 80372760 | intron (NR_033265, intron 2 of 9) | −3458 | NM_001369488 | 284004 | ENSG00000169660 | HEXD |
| chr17 | 80372761 | intron (NR_033265, intron 2 of 9) | −3457 | NM_001369488 | 284004 | ENSG00000169660 | HEXD |
| chr17 | 80372766 | intron (NR_033265, intron 2 of 9) | −3452 | NM_001369488 | 284004 | ENSG00000169660 | HEXD |
| chr17 | 80372767 | intron (NR_033265, intron 2 of 9) | −3451 | NM_001369488 | 284004 | ENSG00000169660 | HEXD |
| chr17 | 80372770 | intron (NR_033265, intron 2 of 9) | −3448 | NM_001369488 | 284004 | ENSG00000169660 | HEXD |
| chr17 | 80372771 | intron (NR_033265, intron 2 of 9) | −3447 | NM_001369488 | 284004 | ENSG00000169660 | HEχD |
| chr17 | 80372793 | intron (NR_033265, intron 2 of 9) | −3425 | NM_001369488 | 284004 | ENSG00000169660 | HEXD |
| chr18 | 57364080 | CpG-11843 | 570 | NM_133459 | 147372 | ENSG00000183287 | CCBE1 |
| chr19 | 591647 | CpG | 1771 | NM_001194 | 610 | ENSG00000099822 | HCN2 |
| chr19 | 591656 | CpG | 1780 | NM_001194 | 610 | ENSG00000099822 | HCN2 |
| chr19 | 591660 | CpG | 1784 | NM_001194 | 610 | ENSG00000099822 | HCN2 |
| chr19 | 591662 | CpG | 1786 | NM_001194 | 610 | ENSG00000099822 | HCN2 |
| chr19 | 591682 | CpG | 1806 | NM_001194 | 610 | ENSG00000099822 | HCN2 |
| chr19 | 591683 | CpG | 1807 | NM_001194 | 610 | ENSG00000099822 | HCN2 |
| chr19 | 591684 | CpG | 1808 | NM_001194 | 610 | ENSG00000099822 | HCN2 |
| chr19 | 591685 | CpG | 1809 | NM_001194 | 610 | ENSG00000099822 | HCN2 |
| chr19 | 591688 | CpG | 1812 | NM_001194 | 610 | ENSG00000099822 | HCN2 |
| chr19 | 18837089 | intron (NM_001098482, intron 1 of 14) | 42604 | NM_001098482 | 23373 | ENSG00000105662 | CRTC1 |
| chr19 | 18837102 | intron (NM_001098482, intron 1 of 14) | 42617 | NM_001098482 | 23373 | ENSG00000105662 | CRTC1 |
| chr20 | 3778162 | intron (NM_004358, intron 1 of 15) | 1781 | NR_136335 | 994 | ENSG00000101224 | CDC25B |
| chr20 | 32156432 | intron (NM_001039709, intron 1 of 10) | 6266 | NM_005093 | 9139 | ENSG00000078699 | CBFA2T2 |
| chr20 | 48099327 | promoter-TSS (NM_004975) | −127 | NM_004975 | 3745 | ENSG00000158445 | KCNB1 |
| chr20 | 48099339 | promoter-TSS (NM_004975) | −139 | NM_004975 | 3745 | ENSG00000158445 | KCNB1 |
| chrX | 36981727 | CpG | −44700 | NM_001013736 | 442444 | ENSG00000198173 | FAM47C |
| chrX | 36981729 | CpG | −44698 | NM_001013736 | 442444 | ENSG00000198173 | FAM47C |
| chr1 | 24392969 | MIRb\|SINE\|MIR | 45556 | NM_152372 | 127294 | ENSG00000142661 | MYOM3 |
| chr1 | 24410522 | MSTA\|LTR\|ERVL-MaLR | 28003 | NM_152372 | 127294 | ENSG00000142661 | MYOM3 |
| chr1 | 24434829 | intron (NM_152372, intron 2 of 36) | 3696 | NM_152372 | 127294 | ENSG00000142661 | MYOM3 |
| chr1 | 24438565 | promoter-TSS (NM_152372) | −40 | NM_152372 | 127294 | ENSG00000142661 | MYOM3 |
| chr1 | 27240357 | promoter-TSS (NM_021969) | 9 | NM_021969 | 8431 | ENSG00000131910 | NR0B2 |
| chr1 | 50513586 | promoter-TSS (NM_001324208) | −41 | NM_001324208 | 1996 | ENSG00000162374 | ELAVL4 |
| chr1 | 50569482 | promoter-TSS (NM_001144776) | 1 | NM_001144776 | 1996 | ENSG00000162374 | ELAVL4 |
| chr1 | 50571262 | promoter-TSS (NM_001144775) | −541 | NM_001144775 | 1996 | ENSG00000162374 | ELAVL4 |
| chr1 | 50571864 | promoter-TSS (NM_001144775) | 61 | NM_001144775 | 1996 | ENSG00000162374 | ELAVL4 |
| chr1 | 50574505 | promoter-TSS (NM_001144774) | 13 | NM_021952 | 1996 | ENSG00000162374 | ELAVL4 |
| chr1 | 50574538 | promoter-TSS (NM_001144774) | 46 | NM_021952 | 1996 | ENSG00000162374 | ELAVL4 |
| chr1 | 50575198 | promoter-TSS (NM_001324217) | −207 | NM_001324215 | 1996 | ENSG00000162374 | ELAVL4 |
| chr1 | 50575278 | promoter-TSS (NM_001324217) | −127 | NM_001324215 | 1996 | ENSG00000162374 | ELAVL4 |
| chr1 | 50575388 | promoter-TSS (NM_001324217) | −17 | NM_001324215 | 1996 | ENSG00000162374 | ELAVL4 |
| chr1 | 50610525 | intron (NR_136725, intron 2 of 4) | 35120 | NM_001324214 | 1996 | ENSG00000162374 | ELAVL4 |
| chr1 | 50642702 | exon (NM_001144774, exon 3 of 7) | 67297 | NM_001324214 | 1996 | ENSG00000162374 | ELAVL4 |
| chr1 | 78445126 | promoter-TSS (NM_001317099) | −337 | NM_001303433 | 8880 | ENSG00000162613 | FUBP1 |
| chr1 | 78511486 | promoter-TSS (NM_017655) | −64 | NM_017655 | 54810 | ENSG00000137960 | GIPC2 |
| chr1 | 114521963 | promoter-TSS (NM_001286353) | −33 | NM_020190 | 56944 | ENSG00000116774 | OLFML3 |
| chr1 | 114521965 | promoter-TSS (NM_001286353) | −31 | NM_020190 | 56944 | ENSG00000116774 | OLFML3 |
| chr1 | 114522013 | promoter-TSS (NM_001286353) | 17 | NM_001286352 | 56944 | ENSG00000116774 | OLFML3 |
| chr1 | 114522595 | exon (NM_001286352, exon 2 of 4) | 599 | NM_001286352 | 56944 | ENSG00000116774 | OLFML3 |
| chr1 | 152956457 | promoter-TSS (NM_001199828) | 0 | NM_001199828 | 6698 | ENSG00000169474 | SPRR1A |
| chr1 | 152957607 | exon (NM_001199828, exon 2 of 2) | 1150 | NM_005987 | 6698 | ENSG00000169474 | SPRR1A |
| chr1 | 153330230 | promoter-TSS (NM_002965) | 0 | NM_002965 | 6280 | ENSG00000163220 | S100A9 |
| chr2 | 74119341 | promoter-TSS (NM_001199893) | −694 | NM_001199893 | 72 | ENSG00000163017 | ACTG2 |
| chr2 | 74119978 | promoter-TSS (NM_001199893) | −57 | NM_001199893 | 72 | ENSG00000163017 | ACTG2 |
| chr2 | 74119994 | promoter-TSS (NM_001199893) | −41 | NM_001199893 | 72 | ENSG00000163017 | ACTG2 |
| chr2 | 74120006 | promoter-TSS (NM_001199893) | −29 | NM_001199893 | 72 | ENSG00000163017 | ACTG2 |
| chr2 | 74120009 | promoter-TSS (NM_001199893) | −26 | NM_001199893 | 72 | ENSG00000163017 | ACTG2 |
| chr2 | 74120035 | promoter-TSS (NM_001199893) | 0 | NM_001199893 | 72 | ENSG00000163017 | ACTG2 |
| chr2 | 95940101 | promoter-TSS (NM_001165978) | 4 | NM_001165978 | 150696 | ENSG00000155066 | PROM2 |
| chr2 | 95940121 | promoter-TSS (NM_001165978) | 24 | NM_001165978 | 150696 | ENSG00000155066 | PROM2 |
| chr2 | 95940140 | promoter-TSS (NM_001165978) | 43 | NM_001165978 | 150696 | ENSG00000155066 | PROM2 |
| chr2 | 95940145 | promoter-TSS (NM_001165978) | 48 | NM_001165978 | 150696 | ENSG00000155066 | PROM2 |
| chr2 | 95940149 | promoter-TSS (NM_001165978) | 52 | NM_001165978 | 150696 | ENSG00000155066 | PROM2 |
| chr2 | 95940627 | MIR3\|SINE\|MIR | 530 | NM_001165978 | 150696 | ENSG00000155066 | PROM2 |
| chr2 | 95943464 | MIRb\|SINE\|MIR | 3367 | NM_001165978 | 150696 | ENSG00000155066 | PROM2 |
| chr2 | 95945514 | exon (NM_001165978, exon 11 of 24) | 5417 | NM_001165978 | 150696 | ENSG00000155066 | PROM2 |
| chr2 | 95954155 | exon (NM_001165978, exon 22 of 24) | −8840 | NM_013434 | 30818 | ENSG00000115041 | KCNIP3 |
| chr2 | 95954163 | exon (NM_001165978, exon 22 of 24) | −8832 | NM_013434 | 30818 | ENSG00000115041 | KCNIP3 |
| chr2 | 169921199 | L1MC5\|LINE\|L1 | −2288 | NM_001289763 | 10170 | ENSG00000073737 | DHRS9 |
| chr2 | 169923445 | promoter-TSS (NM_001289763) | −42 | NM_001289763 | 10170 | ENSG00000073737 | DHRS9 |
| chr2 | 169923487 | promoter-TSS (NM_001289763) | 0 | NM_001289763 | 10170 | ENSG00000073737 | DHRS9 |
| chr2 | 169925966 | 5′ UTR (NM_199204, exon 2 of 6) | 2479 | NM_199204 | 10170 | ENSG00000073737 | DHRS9 |
| chr2 | 169925972 | 5′ UTR (NM_199204, exon 2 of 6) | 2485 | NM_199204 | 10170 | ENSG00000073737 | DHRS9 |
| chr2 | 169925986 | 5′ UTR (NM_199204, exon 2 of 6) | 2499 | NM_199204 | 10170 | ENSG00000073737 | DHRS9 |
| chr2 | 169928910 | promoter-TSS (NM_001142270) | −75 | NM_001142270 | 10170 | ENSG00000073737 | DHRS9 |
| chr2 | 169937517 | promoter-TSS (NM_001142271) | 0 | NM_001142271 | 10170 | ENSG00000073737 | DHRS9 |
| chr2 | 177001240 | promoter-TSS (NR_148241) | 0 | NR_148241 | 401021 | | LOC401021 |
| chr2 | 177001585 | non-coding (NR_038435, exon 1 of 3) | 141 | NR_038435 | 100506783 | | HOXD-AS2 |
| chr2 | 177015022 | promoter-TSS (NR_029609) | 91 | NR_029609 | 406903 | ENSG00000207744 | MIR10B |
| chr2 | 177025519 | CpG | 3584 | NM_006898 | 3232 | ENSG00000128652 | HOXD3 |
| chr2 | 177028705 | 5′ UTR (NM_006898, exon 2 of 4) | 6770 | NM_006898 | 3232 | ENSG00000128652 | HOXD3 |
| chr2 | 182545174 | promoter-TSS (NR_146176) | −30 | NR_146176 | 4760 | ENSG00000162992 | NEUROD1 |
| chr2 | 182545503 | promoter-TSS (NR_146176) | −211 | NR_146175 | 4760 | ENSG00000162992 | NEUROD1 |
| chr2 | 219696414 | promoter-TSS (NM_017431) | 12 | NM_017431 | 53632 | ENSG00000115592 | PRKAG3 |
| chr2 | 219696424 | promoter-TSS (NM_017431) | 2 | NM_017431 | 53632 | ENSG00000115592 | PRKAG3 |
| chr2 | 219696426 | promoter-TSS (NM_017431) | 0 | NM_017431 | 53632 | ENSG00000115592 | PRKAG3 |
| chr2 | 219696439 | promoter-TSS (NM_017431) | −13 | NM_017431 | 53632 | ENSG00000115592 | PRKAG3 |
| chr2 | 219696709 | promoter-TSS (NM_017431) | −283 | NM_017431 | 53632 | ENSG00000115592 | PRKAG3 |
| chr2 | 223916432 | promoter-TSS (NM_080671) | −116 | NM_080671 | 23704 | ENSG00000152049 | KCNE4 |
| chr2 | 223916762 | 5′ UTR (NM_080671, exon 1 of 2) | 214 | NM_080671 | 23704 | ENSG00000152049 | KCNE4 |
| chr2 | 223916818 | exon (NM_080671, exon 1 of 2) | 270 | NM_080671 | 23704 | ENSG00000152049 | KCNE4 |
| chr2 | 233924577 | promoter-TSS (NM_005541) | −375 | NM_005541 | 3635 | ENSG00000168918 | INPP5D |
| chr2 | 233924962 | promoter-TSS (NM_005541) | 10 | NM_001017915 | 3635 | ENSG00000168918 | INPP5D |
| chr2 | 233925089 | exon (NM_005541, exon 1 of 26) | 137 | NM_001017915 | 3635 | ENSG00000168918 | INPP5D |
| chr2 | 233940872 | intron (NM_001017915, intron 1 of 25) | 15920 | NM_001017915 | 3635 | ENSG00000168918 | INPP5D |
| chr2 | 233985212 | intron (NM_001017915, intron 2 of 25) | 60260 | NM_001017915 | 3635 | ENSG00000168918 | INPP5D |
| chr2 | 233986826 | exon (NM_005541, exon 3 of 26) | 61874 | NM_001017915 | 3635 | ENSG00000168918 | INPP5D |
| chr2 | 234053819 | MIRc\|SINE\|MIR | −106298 | NM_198890 | 55054 | ENSG00000085978 | ATG16L1 |
| chr2 | 234054695 | intron (NM_001017915, intron 5 of 25) | −105422 | NM_198890 | 55054 | ENSG00000085978 | ATG16L1 |
| chr2 | 234070270 | exon (NM_005541, exon 10 of 26) | −89847 | NM_198890 | 55054 | ENSG00000085978 | ATG16L1 |
| chr2 | 234070335 | exon (NM_005541, exon 10 of 26) | −89782 | NM_198890 | 55054 | ENSG00000085978 | ATG16L1 |
| chr2 | 234072847 | intron (NM_001017915, intron 11 of 25) | −87270 | NM_198890 | 55054 | ENSG00000085978 | ATG16L1 |
| chr2 | 234078106 | intron (NM_001017915, intron 13 of 25) | −82011 | NM_198890 | 55054 | ENSG00000085978 | ATG16L1 |
| chr2 | 234078567 | intron (NM_001017915, intron 13 of 25) | −81550 | NM_198890 | 55054 | ENSG00000085978 | ATG16L1 |
| chr2 | 234078920 | intron (NM_001017915, intron 14 of 25) | −81197 | NM_198890 | 55054 | ENSG00000085978 | ATG16L1 |
| chr2 | 234103983 | exon (NM_005541, exon 23 of 26) | −56134 | NM_198890 | 55054 | ENSG00000085978 | ATG16L1 |
| chr2 | 234113188 | exon (NM_005541, exon 25 of 26) | −46929 | NM_198890 | 55054 | ENSG00000085978 | ATG16L1 |
| chr3 | 12837871 | promoter-TSS (NM_001162499) | −208 | NM_001162499 | 23066 | ENSG00000144712 | CAND2 |
| chr3 | 12838071 | promoter-TSS (NM_001162499) | −8 | NM_001162499 | 23066 | ENSG00000144712 | CAND2 |
| chr3 | 12838079 | promoter-TSS (NM_001162499) | 0 | NM_001162499 | 23066 | ENSG00000144712 | CAND2 |
| chr3 | 12848532 | intron (NM_012298, intron 2 of 12) | 10453 | NM_012298 | 23066 | ENSG00000144712 | CAND2 |
| chr3 | 12868845 | exon (NM_001162499, exon 13 of 15) | 13004 | NM_001007074 | 6161 | ENSG00000144713 | RPL32 |
| chr3 | 42726911 | promoter-TSS (NM_152393) | −18 | NM_152393 | 131377 | ENSG00000157119 | KLHL40 |
| chr3 | 111717411 | promoter-TSS (NM_013259) | −75 | NM_013259 | 29114 | ENSG00000144834 | TAGLN3 |
| chr3 | 111717486 | promoter-TSS (NM_013259) | 0 | NM_013259 | 29114 | ENSG00000144834 | TAGLN3 |
| chr3 | 111717542 | promoter-TSS (NM_013259) | 56 | NM_013259 | 29114 | ENSG00000144834 | TAGLN3 |
| chr3 | 111717936 | promoter-TSS (NM_001008273) | 29 | NM_001008273 | 29114 | ENSG00000144834 | TAGLN3 |
| chr3 | 111718095 | 5′ UTR (NM_013259, exon 2 of 5) | 188 | NM_001008273 | 29114 | ENSG00000144834 | TAGLN3 |
| chr3 | 111719270 | intron (NM_001008273, intron 1 of 3) | 1363 | NM_001008273 | 29114 | ENSG00000144834 | TAGLN3 |
| chr3 | 111719519 | exon (NM_013259, exon 3 of 5) | 1612 | NM_001008273 | 29114 | ENSG00000144834 | TAGLN3 |
| chr3 | 128200821 | intron (NM_001145661, intron5 of 6) | 5843 | NM_001145662 | 2624 | ENSG00000179348 | GATA2 |
| chr3 | 128206659 | promoter-TSS (NM_001145662) | 5 | NM_001145662 | 2624 | ENSG00000179348 | GATA2 |
| chr3 | 128207273 | promoter-TSS (NM_001145662) | 0 | NM_001145661 | 2624 | ENSG00000179348 | GATA2 |
| chr3 | 128209391 | CpG-18893 | 1445 | NR_125398 | 101927167 | | GATA2-AS1 |
| chr3 | 128211916 | promoter-TSS (NM_032638) | 14 | NM_032638 | 2624 | ENSG00000179348 | GATA2 |
| chr3 | 128211928 | promoter-TSS (NM_032638) | 2 | NM_032638 | 2624 | ENSG00000179348 | GATA2 |
| chr3 | 150481064 | promoter-TSS (NM_005067) | −208 | NM_005067 | 6478 | ENSG00000181788 | SIAH2 |
| chr3 | 150481118 | promoter-TSS (NM_005067) | −262 | NM_005067 | 6478 | ENSG00000181788 | SIAH2 |
| chr3 | 150481164 | promoter-TSS (NM_005067) | −308 | NM_005067 | 6478 | ENSG00000181788 | SIAH2 |
| chr3 | 151154610 | TTS (NM_053002) | 3858 | NM_001178145 | 285313 | ENSG00000152580 | IGSF10 |
| chr3 | 151158459 | promoter-TSS (NM_001178145) | 9 | NM_001178145 | 285313 | ENSG00000152580 | IGSF10 |
| chr3 | 151158468 | promoter-TSS (NM_001178145) | 0 | NM_001178145 | 285313 | ENSG00000152580 | IGSF10 |
| chr3 | 151160838 | exon (NM_178822, exon 5 of 6) | −2370 | NM_001178146 | 285313 | ENSG00000152580 | IGSF10 |
| chr3 | 151176397 | promoter-TSS (NM_178822) | 0 | NM_178822 | 285313 | ENSG00000152580 | IGSF10 |
| chr3 | 181429614 | promoter-TSS (NM_003106) | 1 | NM_003106 | 6657 | ENSG00000181449 | SOX2 |
| chr3 | 181429622 | promoter-TSS (NM_003106) | 9 | NM_003106 | 6657 | ENSG00000181449 | SOX2 |
| chr3 | 185782249 | intron (NM_004454, intron 8 of 12) | 44529 | NM_004454 | 2119 | ENSG00000244405 | ETV5 |
| chr3 | 185783659 | exon (NM_004454, exon 8 of 13) | 43119 | NM_004454 | 2119 | ENSG00000244405 | ETV5 |
| chr3 | 185796794 | intron (NM_004454, intron 7 of 12) | 29984 | NM_004454 | 2119 | ENSG00000244405 | ETV5 |
| chr3 | 185797595 | exon (NM_004454, exon 7 of 13) | 29183 | NM_004454 | 2119 | ENSG00000244405 | ETV5 |
| chr3 | 185823179 | exon (NM_004454, exon 4 of 13) | 3599 | NM_004454 | 2119 | ENSG00000244405 | ETV5 |
| chr3 | 185823413 | intron (NM_004454, intron 2 of 12) | 3365 | NM_004454 | 2119 | ENSG00000244405 | ETV5 |
| chr3 | 185823631 | 5′ UTR (NM_004454, exon 2 of 13) | 3147 | NM_004454 | 2119 | ENSG00000244405 | ETV5 |
| chr3 | 185826233 | CpG | 545 | NM_004454 | 2119 | ENSG00000244405 | ETV5 |
| chr3 | 185826239 | CpG | 539 | NM_004454 | 2119 | ENSG00000244405 | ETV5 |
| chr3 | 185826364 | CpG | 414 | NM_004454 | 2119 | ENSG00000244405 | ETV5 |
| chr3 | 185826618 | CpG | 160 | NM_004454 | 2119 | ENSG00000244405 | ETV5 |
| chr3 | 185826688 | promoter-TSS (NM_004454) | 90 | NM_004454 | 2119 | ENSG00000244405 | ETV5 |
| chr3 | 185826770 | promoter-TSS (NM_004454) | 8 | NM_004454 | 2119 | ENSG00000244405 | ETV5 |
| chr3 | 185826801 | promoter-TSS (NM_004454) | −23 | NM_004454 | 2119 | ENSG00000244405 | ETV5 |
| chr3 | 185828007 | Intergenic | −1229 | NM_004454 | 2119 | ENSG00000244405 | ETV5 |
| chr3 | 187086020 | promoter-TSS (NM_022147) | −53 | NM_022147 | 64108 | ENSG00000136514 | RTP4 |
| chr4 | 6694696 | promoter-TSS (NM_005980) | −809 | NM_005980 | 6286 | ENSG00000163993 | S100P |
| chr4 | 6695505 | promoter-TSS (NM_005980) | 0 | NM_005980 | 6286 | ENSG00000163993 | S100P |
| chr4 | 38796154 | Intergenic | 10158 | NM_003263 | 7096 | ENSG00000174125 | TLR1 |
| chr4 | 38805897 | 5′ UTR (NM_003263, exon 2 of 4) | 415 | NM_003263 | 7096 | ENSG00000174125 | TLR1 |
| chr4 | 38806310 | promoter-TSS (NM_003263) | 2 | NM_003263 | 7096 | ENSG00000174125 | TLR1 |
| chr4 | 38806312 | promoter-TSS (NM_003263) | 0 | NM_003263 | 7096 | ENSG00000174125 | TLR1 |
| chr4 | 38806340 | promoter-TSS (NM_003263) | −28 | NM_003263 | 7096 | ENSG00000174125 | TLR1 |
| chr4 | 38806372 | promoter-TSS (NM_003263) | −60 | NM_003263 | 7096 | ENSG00000174125 | TLR1 |
| chr4 | 38806437 | promoter-TSS (NM_003263) | −125 | NM_003263 | 7096 | ENSG00000174125 | TLR1 |
| chr4 | 38807054 | promoter-TSS (NM_003263) | −742 | NM_003263 | 7096 | ENSG00000174125 | TLR1 |
| chr4 | 38807099 | promoter-TSS (NM_003263) | −787 | NM_003263 | 7096 | ENSG00000174125 | TLR1 |
| chr4 | 38858338 | promoter-TSS (NM_006068) | 0 | NM_006068 | 10333 | ENSG00000174130 | TLR6 |
| chr4 | 41258330 | promoter-TSS (NM_004181) | 314 | NR_102709 | 101410542 | ENSG00000251173 | UCHL1-AS1 |
| chr4 | 41258686 | promoter-TSS (NM_004181) | −42 | NR_102709 | 101410542 | ENSG00000251173 | UCHL1-AS1 |
| chr4 | 41258750 | promoter-TSS (NM_004181) | −95 | NM_004181 | 7345 | ENSG00000154277 | UCHL1 |
| chr4 | 41258825 | promoter-TSS (NM_004181) | −20 | NM_004181 | 7345 | ENSG00000154277 | UCHL1 |
| chr4 | 41258828 | promoter-TSS (NM_004181) | −17 | NM_004181 | 7345 | ENSG00000154277 | UCHL1 |
| chr4 | 41258849 | promoter-TSS (NM_004181) | 4 | NM_004181 | 7345 | ENSG00000154277 | UCHL1 |
| chr4 | 41258850 | promoter-TSS (NM_004181) | 5 | NM_004181 | 7345 | ENSG00000154277 | UCHL1 |
| chr4 | 41258857 | promoter-TSS (NM_004181) | 12 | NM_004181 | 7345 | ENSG00000154277 | UCHL1 |
| chr4 | 41258862 | promoter-TSS (NM_004181) | 17 | NM_004181 | 7345 | ENSG00000154277 | UCHL1 |
| chr4 | 41258863 | promoter-TSS (NM_004181) | 18 | NM_004181 | 7345 | ENSG00000154277 | UCHLI |
| chr4 | 41262556 | intron (NM_004181, intron 3 of 8) | 3711 | NM_004181 | 7345 | ENSG00000154277 | UCHL1 |
| chr4 | 41263656 | exon (NM_004181, exon 5 of 9) | 4811 | NM_004181 | 7345 | ENSG00000154277 | UCHLI |
| chr4 | 48899120 | intron (NR_104589, intron 2 of 3) | 9571 | NM_001014446 | 132299 | ENSG00000145247 | OCIAD2 |
| chr4 | 48899765 | exon (NM_001014446, exon 4 of 7) | 8926 | NM_001014446 | 132299 | ENSG00000145247 | OCIAD2 |
| chr4 | 48908644 | promoter-TSS (NM_001014446) | 47 | NM_001014446 | 132299 | ENSG00000145247 | OCIAD2 |
| chr4 | 48908674 | promoter-TSS (NM_001014446) | 17 | NM_001014446 | 132299 | ENSG00000145247 | OCIAD2 |
| chr4 | 48908691 | promoter-TSS (NM_001014446) | 0 | NM_001014446 | 132299 | ENSG00000145247 | OCIAD2 |
| chr4 | 48908705 | promoter-TSS (NM_001014446) | −14 | NM_001014446 | 132299 | ENSG00000145247 | OCIAD2 |
| chr4 | 48908715 | promoter-TSS (NM_001014446) | −24 | NM_001014446 | 132299 | ENSG00000145247 | OCIAD2 |
| chr4 | 48908745 | promoter-TSS (NM_001014446) | 0 | NM_152398 | 132299 | ENSG00000145247 | OCIAD2 |
| chr4 | 48908854 | promoter-TSS (NM_001014446) | −109 | NM_001286773 | 132299 | ENSG00000145247 | OC1AD2 |
| chr4 | 84030896 | promoter-TSS (NM_001130716) | 46 | NM_001130716 | 51316 | ENSG00000145287 | PLAC8 |
| chr4 | 84035768 | promoter-TSS (NM_016619) | 14 | NM_016619 | 51316 | ENSG00000145287 | PLAC8 |
| chr4 | 84035789 | promoter-TSS (NM_016619) | −7 | NM_001130715 | 51316 | ENSG00000145287 | PLAC8 |
| chr4 | 84035855 | promoter-TSS (NM_016619) | −73 | NM_001130715 | 51316 | ENSG00000145287 | PLAC8 |
| chr4 | 84058128 | Intergenic | −22346 | NM_001130715 | 51316 | ENSG00000145287 | PLAC8 |
| chr4 | 177158625 | intron (NM_080874, intron 1 of 6) | 31648 | NM_080874 | 140458 | ENSG00000164122 | ASB5 |
| chr4 | 177162727 | intron (NM_080874, intron 1 of 6) | 27546 | NM_080874 | 140458 | ENSG00000164122 | ASB5 |
| chr4 | 177170609 | intron (NM_080874, intron 1 of 6) | 19664 | NM_080874 | 140458 | ENSG00000164122 | ASB5 |
| chr4 | 177190273 | promoter-TSS (NM_080874) | 0 | NM_080874 | 140458 | ENSG00000164122 | ASB5 |
| chr4 | 177198622 | Intergenic | −8349 | NM_080874 | 140458 | ENSG00000164122 | ASB5 |
| chr5 | 102089585 | promoter-TSS (NM_001319943) | −802 | NM_001319943 | 5066 | ENSG00000145730 | PAM |
| chr5 | 102090271 | promoter-TSS (NM_001364586) | −116 | NM_001319943 | 5066 | ENSG00000145730 | PAM |
| chr5 | 102090387 | promoter-TSS (NM_001364586) | 0 | NM_001319943 | 5066 | ENSG00000145730 | PAM |
| chr5 | 102090918 | promoter-TSS (NM_001364586) | 9 | NR_157231 | 5066 | ENSG00000145730 | PAM |
| chr5 | 102090947 | promoter-TSS (NM_001364586) | 38 | NR_157231 | 5066 | ENSG00000145730 | PAM |
| chr5 | 102143661 | promoter-TSS (NM_001364582) | −141 | NM_001364582 | 5066 | ENSG00000145730 | PAM |
| chr5 | 102143667 | promoter-TSS (NM_001364582) | −135 | NM_001364582 | 5066 | ENSG00000145730 | PAM |
| chr5 | 102200141 | intron (NM_001364582, intron 1 of 24) | −1286 | NM_138821 | 5066 | ENSG00000145730 | PAM |
| chr5 | 102200848 | promoter-TSS (NM_138822) | −579 | NM_138821 | 5066 | ENSG00000145730 | PAM |
| chr5 | 102200914 | promoter-TSS (NM_138822) | −513 | NM_138821 | 5066 | ENSG00000145730 | PAM |
| chr5 | 102201083 | promoter-TSS (NM_138822) | −344 | NM_138821 | 5066 | ENSG00000145730 | PAM |
| chr5 | 102201330 | promoter-TSS (NM_138822) | −97 | NM_138821 | 5066 | ENSG00000145730 | PAM |
| chr5 | 102201341 | promoter-TSS (NM_138822) | −86 | NM_138821 | 5066 | ENSG00000145730 | PAM |
| chr5 | 102201423 | promoter-TSS (NM_138822) | −4 | NM_138821 | 5066 | ENSG00000145730 | PAM |
| chr5 | 102201427 | promoter-TSS (NM_138822) | 0 | NM_138821 | 5066 | ENSG00000145730 | PAM |
| chr5 | 102201614 | non-coding (NR_033440, exon 1 of 24) | 187 | NM_138822 | 5066 | ENSG00000145730 | PAM |
| chr5 | 102201661 | non-coding (NR_033440, exon 1 of 24) | 234 | NM_138822 | 5066 | ENSG00000145730 | PAM |
| chr5 | 102201720 | non-coding (NR_033440, exon 1 of 24) | 293 | NM_138822 | 5066 | ENSG00000145730 | PAM |
| chr5 | 102202191 | intron (NM_001364584, intron 2 of 25) | 764 | NM_138822 | 5066 | ENSG00000145730 | PAM |
| chr5 | 102231517 | intron (NR_033440, intron 2 of 23) | 30090 | NM_138822 | 5066 | ENSG00000145730 | PAM |
| chr5 | 102285181 | exon (NM_001364582, exon 10 of 25) | 83754 | NM_138822 | 5066 | ENSG00000145730 | PAM |
| chr5 | 102295601 | exon (NM_001364582, exon 13 of 25) | 94174 | NM_138822 | 5066 | ENSG00000145730 | PAM |
| chr5 | 102325375 | intron (NM_001364591, intron 15 of 24) | 123948 | NM_138822 | 5066 | ENSG00000145730 | PAM |
| chr5 | 102343057 | intron (NM_001364584, intron 19 of 25) | 112685 | NM_001317954 | 54826 | ENSG00000145723 | GIN1 |
| chr5 | 102343146 | exon (NM_001364582, exon 20 of 25) | 112596 | NM_001317954 | 54826 | ENSG00000145723 | GIN1 |
| chr5 | 102343163 | exon (NM_001364582, exon 20 of 25) | 112579 | NM_001317954 | 54826 | ENSG00000145723 | GIN1 |
| chr5 | 102343173 | exon (NM_001364582, exon 20 of 25) | 112569 | NM_001317954 | 54826 | ENSG00000145723 | GIN1 |
| chr6 | 34523991 | promoter-TSS (NM_001252294) | 15 | NM_012391 | 25803 | ENSG00000124664 | SPDEF |
| chr6 | 34524010 | promoter-TSS (NM_001252294) | 0 | NM_001252294 | 25803 | ENSG00000124664 | SPDEF |
| chr6 | 112451090 | exon (NM_002290, exon 30 of 39) | 42396 | NM_001033564 | 619208 | ENSG00000203778 | FAM229B |
| chr6 | 112451261 | intron (NM_001105206, intron 29 of 38) | 42567 | NM_001033564 | 619208 | ENSG00000203778 | FAM229B |
| chr6 | 112460378 | exon (NM_002290, exon 24 of 39) | 51684 | NM_001033564 | 619208 | ENSG00000203778 | FAM229B |
| chr6 | 112486266 | exon (NM_002290, exon 13 of 39) | −71431 | NR_121193 | 101927640 | | LOC101927640 |
| chr6 | 112500604 | intron (NM_001105206, intron 9 of 38) | −57093 | NR_121193 | 101927640 | | LOC101927640 |
| chr6 | 112512866 | exon (NM_002290, exon 6 of 39) | −44831 | NR_121193 | 101927640 | | LOC101927640 |
| chr6 | 112512903 | exon (NM_002290, exon 6 of 39) | −44794 | NR_121193 | 101927640 | | LOC101927640 |
| chr6 | 112512913 | exon (NM_002290, exon 6 of 39) | −44784 | NR_121193 | 101927640 | | LOC101927640 |
| chr6 | 112537550 | exon (NM_002290, exon 3 of 39) | −20147 | NR_121193 | 101927640 | | LOC101927640 |
| chr6 | 112537855 | intron (NM_001105206, intron 2 of 38) | −19842 | NR_121193 | 101927640 | | LOC101927640 |
| chr6 | 112575587 | promoter-TSS (NM_002290) | 82 | NM_001105207 | 3910 | ENSG00000112769 | LAMA4 |
| chr6 | 112575640 | promoter-TSS (NM_002290) | 29 | NM_001105207 | 3910 | ENSG00000112769 | LAMA4 |
| chr6 | 112575674 | promoter-TSS (NM_002290) | −5 | NM_001105206 | 3910 | ENSG00000112769 | LAMA4 |
| chr6 | 112575680 | promoter-TSS (NM_002290) | −11 | NM_001105206 | 3910 | ENSG00000112769 | LAMA4 |
| chr6 | 112575690 | promoter-TSS (NM_002290) | −21 | NM_001105206 | 3910 | ENSG00000112769 | LAMA4 |
| chr6 | 112575702 | promoter-TSS (NM_002290) | −33 | NM_001105206 | 3910 | ENSG00000112769 | LAMA4 |
| chr6 | 112575749 | promoter-TSS (NM_002290) | 68 | NM_001105209 | 3910 | ENSG00000112769 | LAMA4 |
| chr6 | 112575812 | promoter-TSS (NM_002290) | 5 | NM_001105209 | 3910 | ENSG00000112769 | LAMA4 |
| chr6 | 112575817 | promoter-TSS (NM_002290) | 0 | NM_001105209 | 3910 | ENSG00000112769 | LAMA4 |
| chr6 | 112575824 | promoter-TSS (NM_002290) | −7 | NM_001105208 | 3910 | ENSG00000112769 | LAMA4 |
| chr6 | 112576041 | promoter-TSS (NM_002290) | −224 | NM_001105208 | 3910 | ENSG00000112769 | LAMA4 |
| chr7 | 148508815 | intron (NM_004456, intron 15 of 19) | 71662 | NM_001203249 | 2146 | ENSG00000106462 | EZH2 |
| chr7 | 148580501 | promoter-TSS (NM_001203249) | −24 | NM_001203249 | 2146 | ENSG00000106462 | EZH2 |
| chr7 | 148581151 | promoter-TSS (NM_001203249) | 132 | NM_152998 | 2146 | ENSG00000106462 | EZH2 |
| chr7 | 148581237 | promoter-TSS (NM_001203247) | 46 | NM_152998 | 2146 | ENSG00000106462 | EZH2 |
| chr7 | 148581240 | promoter-TSS (NM_001203247) | 43 | NM_152998 | 2146 | ENSG00000106462 | EZH2 |
| chr7 | 148581270 | promoter-TSS (NM_001203247) | 13 | NM_152998 | 2146 | ENSG00000106462 | EZH2 |
| chr7 | 148581275 | promoter-TSS (NM_001203247) | 8 | NM_152998 | 2146 | ENSG00000106462 | EZH2 |
| chr7 | 148581279 | promoter-TSS (NM_001203247) | 4 | NM_152998 | 2146 | ENSG00000106462 | EZH2 |
| chr7 | 148581313 | promoter-TSS (NM_001203247) | 28 | NM_004456 | 2146 | ENSG00000106462 | EZH2 |
| chr8 | 17169082 | intron (NM_004686, intron 8 of 13) | 64715 | NM_152415 | 137492 | ENSG00000155975 | VPS37A |
| chr8 | 17169272 | intron (NM_004686, intron 8 of 13) | 64905 | NM_152415 | 137492 | ENSG00000155975 | VPS37A |
| chr8 | 17206466 | exon (NM_004686, exon 5 of 14) | 64294 | NM_004686 | 9108 | ENSG00000003987 | MTMR7 |
| chr8 | 17206646 | intron (NM_004686, intron 4 of 13) | 64114 | NM_004686 | 9108 | ENSG00000003987 | MTMR7 |
| chr8 | 17206894 | L2c\|LINE\|L2 | 63866 | NM_004686 | 9108 | ENSG00000003987 | MTMR7 |
| chr8 | 17270716 | promoter-TSS (NM_004686) | 44 | NM_004686 | 9108 | ENSG00000003987 | MTMR7 |
| chr8 | 17270736 | promoter-TSS (NM_004686) | 24 | NM_004686 | 9108 | ENSG00000003987 | MTMR7 |
| chr8 | 17270937 | promoter-TSS (NM_004686) | −177 | NM_004686 | 9108 | ENSG00000003987 | MTMR7 |
| chr8 | 53045604 | intron (NM_001352827, intron 21 of 26) | −61218 | NR_134310 | 101929341 | ENSG00000253551 | LOC101929341 |
| chr8 | 53045678 | intron (NM_001352827, intron 21 of 26) | −61144 | NR_134310 | 101929341 | ENSG00000253551 | LOC101929341 |
| chr8 | 53050272 | intron (NM_001352827, intron 18 of 26) | −56550 | NR_134310 | 101929341 | ENSG00000253551 | LOC101929341 |
| chr8 | 53130239 | 5′ UTR (NM_001352857, exon 6 of 26) | 23417 | NR_134310 | 101929341 | ENSG00000253551 | LOC101929341 |
| chr8 | 5313028 | 5′ UTR (NM_001352857, exon 6 of 26) | 23459 | NR_134310 | 101929341 | ENSG00000253551 | LOC101929341 |
| chr8 | 53130282 | 5′ UTR (NM_001352857, exon 6 of 26) | 23460 | NR_134310 | 101929341 | ENSG00000253551 | LOC101929341 |
| chr8 | 53134555 | intron (NM_001352838, intron 2 of 23) | 26549 | NM_001352869 | 9705 | ENSG00000147488 | ST18 |
| chr8 | 53159124 | non-coding (NR_148195, exon 3 of 4) | 1980 | NM_001352869 | 9705 | ENSG00000147488 | ST18 |
| chr8 | 53160986 | non-coding (NR_148196, exon 1 of 2) | 118 | NM_001352869 | 9705 | ENSG00000147488 | ST18 |
| chr8 | 53161872 | promoter-TSS (NM_001352856) | 85 | NM_001352854 | 9705 | ENSG00000147488 | ST18 |
| chr8 | 53166808 | 5′ UTR (NM_001352839, exon 1 of 23) | 144 | NM_001352859 | 9705 | ENSG00000147488 | ST18 |
| chr8 | 53322203 | promoter-TSS (NM_001352883) | 13 | NM_001352884 | 9705 | ENSG00000147488 | ST18 |
| chr8 | 53322209 | promoter-TSS (NM_001352883) | 7 | NM_001352884 | 9705 | ENSG00000147488 | ST18 |
| chr8 | 53322216 | promoter-TSS (NM_001352883) | 0 | NM_001352884 | 9705 | ENSG00000147488 | ST18 |
| chr8 | 53322232 | promoter-TSS (NM_001352883) | −16 | NM_001352883 | 9705 | ENSG00000147488 | ST18 |
| chr8 | 53322252 | promoter-TSS (NM_001352826) | −36 | NM_001352883 | 9705 | ENSG00000147488 | ST18 |
| chr8 | 53322265 | promoter-TSS (NM_001352826) | −49 | NM_001352883 | 9705 | ENSG00000147488 | ST18 |
| chr8 | 53322339 | promoter-TSS (NM_001352826) | 0 | NM_001352880 | 9705 | ENSG00000147488 | ST18 |
| chr8 | 53322342 | promoter-TSS (NM_001352826) | −3 | NM_001352831 | 9705 | ENSG00000147488 | ST18 |
| chr8 | 53322405 | promoter-TSS (NM_001352826) | −66 | NM_001352831 | 9705 | ENSG00000147488 | ST18 |
| chr8 | 53373419 | Intergenic | −51080 | NM_001352831 | 9705 | ENSG00000147488 | ST18 |
| chr8 | 104152838 | promoter-TSS (NM_001024372) | −15 | NM_001364874 | 79870 | ENSG00000164929 | BAALC |
| chr8 | 104152841 | promoter-TSS (NM_001024372) | −12 | NM_001364874 | 79870 | ENSG00000164929 | BAALC |
| chr8 | 104152887 | promoter-TSS (NM_001024372) | 34 | NM_024812 | 79870 | ENSG00000164929 | BAALC |
| chr8 | 104153026 | TTS (NR_145429) | 173 | NM_024812 | 79870 | ENSG00000164929 | BAALC |
| chr8 | 104165267 | intron (NM_001024372, intron 1 of 1) | −1475 | NR_036106 | 100422992 | ENSG00000265657 | MIR3151 |
| chr8 | 104220320 | intron (NM_001364874, intron 2 of 3) | −36052 | NR_109954 | 100499183 | ENSG00000247081 | BAALC-AS1 |
| chr8 | 104225459 | intron (NM_001364874, intron 3 of 3) | −41191 | NR_109954 | 100499183 | ENSG00000247081 | BAALC-AS1 |
| chr8 | 104225539 | intron (NM_001364874, intron 3 of 3) | −41271 | NR_109954 | 100499183 | ENSG00000247081 | BAALC-AS1 |
| chr10 | 69597717 | 5′ UTR (NM_021800, exon 1 of 5) | 120 | NM_201262 | 56521 | ENSG00000108176 | DNAJC12 |
| chr10 | 69597721 | 5′ UTR (NM_021800, exon 1 of 5) | 116 | NM_201262 | 56521 | ENSG00000108176 | DNAJC12 |
| chr10 | 69597754 | promoter-TSS (NM_021800) | 83 | NM_201262 | 56521 | ENSG00000108176 | DNAJC12 |
| chr10 | 69597822 | promoter-TSS (NM_021800) | 15 | NM_201262 | 56521 | ENSG00000108176 | DNAJC12 |
| chr10 | 69597824 | promoter-TSS (NM_021800) | 13 | NM_201262 | 56521 | ENSG00000108176 | DNAJC12 |
| chr10 | 88428106 | promoter-TSS (NM_001080116) | −62 | NM_001171610 | 11155 | ENSG00000122367 | LDB3 |
| chr10 | 88428270 | promoter-TSS (NM_001080116) | 49 | NM_001080116 | 11155 | ENSG00000122367 | LDB3 |
| chr10 | 88428326 | 5′ UTR (NM_001080116, exon 1 of 8) | 105 | NM_001080116 | 11155 | ENSG00000122367 | LDB3 |
| chr10 | 88428328 | 5′ UTR (NM_001080116, exon 1 of 8) | 107 | NM_001080116 | 11155 | ENSG00000122367 | LDB3 |
| chr10 | 88428349 | exon (NM_001080116, exon 1 of 8) | 128 | NM_001080116 | 11155 | ENSG00000122367 | LDB3 |
| chr10 | 88478387 | exon (NM_007078, exon 12 of 14) | −37909 | NM_004329 | 657 | ENSG00000107779 | BMPR1A |
| chr10 | 92680933 | promoter-TSS (NM_014391) | −1 | NM_014391 | 27063 | ENSG00000148677 | ANKRD1 |
| chr11 | 60869767 | promoter-TSS (NM_014207) | −94 | NM_014207 | 921 | ENSG00000110448 | CD5 |
| chr11 | 60869881 | promoter-TSS (NM_014207) | 20 | NM_014207 | 921 | ENSG00000110448 | CD5 |
| chr11 | 67350966 | promoter-TSS (NM_000852) | 0 | NM_000852 | 2950 | ENSG00000084207 | GSTP1 |
| chr11 | 67351113 | 5′ UTR (NM_000852, exon 1 of 7) | 147 | NM_000852 | 2950 | ENSG00000084207 | GSTP1 |
| chr11 | 67351193 | 5′ UTR (NM_000852, exon 1 of 7) | 227 | NM_000852 | 2950 | ENSG00000084207 | GSTP1 |
| chr11 | 67351495 | CpG | 529 | NM_000852 | 2950 | ENSG00000084207 | GSTP1 |
| chr11 | 67351496 | CpG | 530 | NM_000852 | 2950 | ENSG00000084207 | GSTP1 |
| chr11 | 67351505 | exon (NM_000852, exon 2 of 7) | 539 | NM_000852 | 2950 | ENSG00000084207 | GSTP1 |
| chr11 | 67351826 | CpG-4453 | 860 | NM_000852 | 2950 | ENSG00000084207 | GSTP1 |
| chr11 | 67352166 | intron (NM_000852, intron 4 of 6) | 1200 | NM_000852 | 2950 | ENSG00000084207 | GSTP1 |
| chr11 | 67353475 | exon (NM_000852, exon 6 of 7) | 2509 | NM_000852 | 2950 | ENSG00000084207 | GSTP1 |
| chr11 | 87908501 | promoter-TSS (NM_022337) | 10 | NM_022337 | 23682 | ENSG00000123892 | RAB38 |
| chr11 | 87908502 | promoter-TSS (NM_022337) | 9 | NM_022337 | 23682 | ENSG00000123892 | RAB38 |
| chr11 | 87908535 | promoter-TSS (NM_022337) | −24 | NM_022337 | 23682 | ENSG00000123892 | RAB38 |
| chr11 | 112831897 | promoter-TSS (NM_001242608) | −145 | NM_001242608 | 4684 | ENSG00000149294 | NCAM1 |
| chr11 | 112831990 | promoter-TSS (NM_001242608) | −52 | NM_001242608 | 4684 | ENSG00000149294 | NCAM1 |
| chr11 | 112832024 | promoter-TSS (NM_001242608) | −18 | NM_001242608 | 4684 | ENSG00000149294 | NCAM1 |
| chr11 | 112832047 | promoter-TSS (NM_001242608) | 5 | NM_001076682 | 4684 | ENSG00000149294 | NCAM1 |
| chr11 | 112832057 | promoter-TSS (NM_001242608) | 15 | NM_001076682 | 4684 | ENSG00000149294 | NCAM1 |
| chr11 | 112832058 | promoter-TSS (NM_001242608) | 16 | NM_001076682 | 4684 | ENSG00000149294 | NCAM1 |
| chr11 | 112832059 | promoter-TSS (NM_001242608) | 17 | NM_001076682 | 4684 | ENSG00000149294 | NCAM1 |
| chr11 | 112832061 | promoter-TSS (NM_001242608) | 19 | NM_001076682 | 4684 | ENSG00000149294 | NCAM1 |
| chr11 | 112832102 | promoter-TSS (NM_001242608) | 60 | NM_001076682 | 4684 | ENSG00000149294 | NCAM1 |
| chr11 | 113001316 | intron (NM_181351, intron 2 of 19) | 143207 | NR_034101 | 100288346 | ENSG00000227487 | NCAM1-AS1 |
| chr11 | 113003550 | intron (NM_181351, intron 2 of 19) | 140973 | NR_034101 | 100288346 | ENSG00000227487 | NCAM1-AS1 |
| chr11 | 113067815 | THE1C\|LTR\|ERVL-MaLR | 76708 | NR_034101 | 100288346 | ENSG00000227487 | NCAM1-AS1 |
| chr11 | 113072979 | (TTTTG)n\|Simple_repeat\|Simple_repeat | 71544 | NR_034101 | 100288346 | ENSG00000227487 | NCAM1-AS1 |
| chr11 | 113072998 | intron (NM_181351, intron 2 of 19) | 71525 | NR_034101 | 100288346 | ENSG00000227487 | NCAM1-AS1 |
| chr11 | 113102002 | intron (NM_001242607, intron 11 of 20) | 42521 | NR_034101 | 100288346 | ENSG00000227487 | NCAM1-AS1 |
| chr11 | 113102884 | exonfNM_001242608, exon 11 of 17) | 41639 | NR_034101 | 100288346 | ENSG00000227487 | NCAM1-AS1 |
| chr11 | 113102947 | exon (NM_001242608, exon 11 of 17) | 41576 | NR_034101 | 100288346 | ENSG00000227487 | NCAM1-AS1 |
| chr11 | 113103415 | exon (NM_001242608, exon 12 of 17) | 41108 | NR_034101 | 100288346 | ENSG00000227487 | NCAM1-AS1 |
| chr11 | 113103417 | exon (NM_001242608, exon 12 of 17) | 41106 | NR_034101 | 100288346 | ENSG00000227487 | NCAM1-AS1 |
| chr11 | 113103766 | intron (NM_181351, intron 13 of 19) | 40757 | NR_034101 | 100288346 | ENSG00000227487 | NCAM1-AS1 |
| chr11 | 113103880 | exon (NM_001242608, exon 13 of 17) | 40643 | NR_034101 | 100288346 | ENSG00000227487 | NCAM1-AS1 |
| chr11 | 113111129 | intron (NM_001076682, intron 15 of 19) | 33394 | NR_034101 | 100288346 | ENSG00000227487 | NCAM1-AS1 |
| chr11 | 113116730 | intron (NM_001076682, intron 17 of 19) | 27793 | NR_034101 | 100288346 | ENSG00000227487 | NCAM1-AS1 |
| chr11 | 113126572 | exon (NM_001242608, exon 15 of 17) | 17951 | NR_034101 | 100288346 | ENSG00000227487 | NCAM1-AS1 |
| chr11 | 113130713 | intron (NM_181351, intron 16 of 19) | 13810 | NR_034101 | 100288346 | ENSG00000227487 | NCAM1-AS1 |
| chr11 | 113130880 | exon (NM_001242608, exon 16 of 17) | 13643 | NR_034101 | 100288346 | ENSG00000227487 | NCAM1-AS1 |
| chr11 | 113140591 | intron (NR_034101, intron 2 of 2) | 3932 | NR_034101 | 100288346 | ENSG00000227487 | NCAM1-AS1 |
| chr11 | 113143642 | intron (NM_181351, intron 19 of 19) | 881 | NR_034101 | 100288346 | ENSG00000227487 | NCAM1-AS1 |
| chr11 | 133938720 | promoter-TSS (NM_001205329) | 0 | NM_001205329 | 83700 | ENSG00000166086 | JAM3 |
| chr11 | 133938866 | promoter-TSS (NM_032801) | −1 | NM_032801 | 83700 | ENSG00000166086 | JAM3 |
| chr11 | 133938880 | promoter-TSS (NM_032801) | 13 | NM_032801 | 83700 | ENSG00000166086 | JAM3 |
| chr11 | 133996134 | intron (NM_032801, intron 1 of 8) | 25024 | NR_145790 | 109623486 | | SNORD153 |
| chr11 | 134009435 | intron (NM_032801, intron 1 of 8) | 11723 | NR_145790 | 109623486 | | SNORD153 |
| chr11 | 134017593 | intron (NM_032801, intron 6 of 8) | 3565 | NR_145790 | 109623486 | | SNORD153 |
| chr11 | 7022809 | promoter-TSS (NM_001975) | −835 | NM_001975 | 2026 | ENSG00000111674 | ENO2 |
| chr11 | 7023391 | promoter-TSS (NM_001975) | −253 | NM_001975 | 2026 | ENSG00000111674 | ENO2 |
| chr11 | 7023514 | promoter-TSS (NM_001975) | −130 | NM_001975 | 2026 | ENSG00000111674 | ENO2 |
| chr11 | 7023602 | promoter-TSS (NM_001975) | −42 | NM_001975 | 2026 | ENSG00000111674 | ENO2 |
| chr11 | 7023621 | promoter-TSS (NM_001975) | −23 | NM_001975 | 2026 | ENSG00000111674 | ENO2 |
| chr12 | 7023653 | promoter-TSS (NM_001975) | 9 | NM_001975 | 2026 | ENSG00000111674 | ENO2 |
| chr12 | 7023658 | promoter-TSS (NM_001975) | 14 | NM_001975 | 2026 | ENSG00000111674 | ENO2 |
| chr12 | 7023661 | promoter-TSS (NM_001975) | 17 | NM_001975 | 2026 | ENSG00000111674 | ENO2 |
| chr12 | 7023674 | promoter-TSS (NM_001975) | 30 | NM_001975 | 2026 | ENSG00000111674 | ENO2 |
| chr12 | 7024271 | TTS (NM_001135217) | 627 | NM_001975 | 2026 | ENSG00000111674 | ENO2 |
| chr12 | 7026393 | intron (NM_001975, intron 5 of 11) | 2749 | NM_001975 | 2026 | ENSG00000111674 | ENO2 |
| chr11 | 7028638 | exon (NM_001975, exon 8 of 12) | −4884 | NM_001007026 | 1822 | ENSG00000111676 | ATN1 |
| chr11 | 7030644 | exon (NM_001975, exon 9 of 12) | −2878 | NM_001007026 | 1822 | ENSG00000111676 | ATN1 |
| chr11 | 7031052 | intron (NM_001975, intron 9 of 11) | −2470 | NM_001007026 | 1822 | ENSG00000111676 | ATN1 |
| chr11 | 24983162 | intron (NM_001178092, intron 7 of 8) | 71894 | NM_001178094 | 586 | ENSG00000060982 | BCAT1 |
| chr12 | 25055130 | promoter-TSS (NM_001178094) | −74 | NM_001178094 | 586 | ENSG00000060982 | BCAT1 |
| chr12 | 25055222 | promoter-TSS (NM_001178094) | −166 | NM_001178094 | 586 | ENSG00000060982 | BCAT1 |
| chr12 | 25055909 | promoter-TSS (NM_001178094) | −8 | NM_001178093 | 586 | ENSG00000060982 | BCAT1 |
| chr12 | 25101877 | promoter-TSS (NM_001178091) | 58 | NM_001178092 | 586 | ENSG00000060982 | BCAT1 |
| chr12 | 25101883 | promoter-TSS (NM_001178091) | 52 | NM_001178092 | 586 | ENSG00000060982 | BCAT1 |
| chr11 | 25101895 | promoter-TSS (NM_001178091) | 40 | NM_001178092 | 586 | ENSG00000060982 | BCAT1 |
| chr11 | 25101920 | promoter-TSS (NM_001178091) | 15 | NM_001178092 | 586 | ENSG00000060982 | BCAT1 |
| chr12 | 25102293 | promoter-TSS (NM_001178091) | −358 | NM_001178091 | 586 | ENSG00000060982 | BCAT1 |
| chr12 | 25173836 | intron (NM_001321724, intron 8 of 17) | 23427 | NM_001321724 | 645177 | | LOC645177 |
| chr12 | 25186593 | intron (NM_001321724, intron 13 of 17) | −18469 | NM_001366542 | 4033 | ENSG00000118308 | LRMP |
| chr12 | 25188314 | intron (NM_001321724, intron 14 of 17) | −16748 | NM_001366542 | 4033 | ENSG00000118308 | LRMP |
| chr12 | 25205089 | promoter-TSS (NM_001366542) | 27 | NR_159366 | 4033 | ENSG00000118308 | LRMP |
| chr12 | 25205105 | promoter-TSS (NM_001366542) | 43 | NR_159366 | 4033 | ENSG00000118308 | LRMP |
| chr12 | 25205106 | promoter-TSS (NM_001366542) | 44 | NR_159366 | 4033 | ENSG00000118308 | LRMP |
| chr12 | 25205346 | promoter-TSS (NM_001204126) | −194 | NM_001204126 | 4033 | ENSG00000118308 | LRMP |
| chr11 | 25205421 | promoter-TSS (NM_001204126) | −119 | NM_001204126 | 4033 | ENSG00000118308 | LRMP |
| chr11 | 25205476 | promoter-TSS (NM_001204126) | −64 | NM_001204126 | 4033 | ENSG00000118308 | LRMP |
| chr12 | 25205499 | promoter-TSS (NM_001204126) | −41 | NM_001204126 | 4033 | ENSG00000118308 | LRMP |
| chr11 | 25205534 | promoter-TSS (NM_001204126) | −6 | NM_001204126 | 4033 | ENSG00000118308 | LRMP |
| chr12 | 25205574 | promoter-TSS (NM_001204126) | 34 | NM_001204127 | 4033 | ENSG00000118308 | LRMP |
| chr12 | 25205779 | TTS (NM_001321724) | 239 | NM_001204127 | 4033 | ENSG00000118308 | LRMP |
| chr12 | 25228574 | intron (NR_159367, intron 5 of 19) | 23034 | NM_001204127 | 4033 | ENSG00000118308 | LRMP |
| chr11 | 25242608 | exon (NM_001366542, exon 12 of 21) | 37068 | NM_001204127 | 4033 | ENSG00000118308 | LRMP |
| chr11 | 25249767 | exon (NM_001366542, exon 14 of 21) | 44227 | NM_001204127 | 4033 | ENSG00000118308 | LRMP |
| chr12 | 25249792 | exon (NM_001366542, exon 14 of 21) | 44252 | NM_001204127 | 4033 | ENSG00000118308 | LRMP |
| chr12 | 25257163 | intron (NR_159367, intron 18 of 19) | 51623 | NM_001204127 | 4033 | ENSG00000118308 | LRMP |
| chr12 | 66582559 | promoter-TSS (NM_001142523) | −335 | NM_007199 | 11213 | ENSG00000090376 | IRAK3 |
| chr12 | 66582875 | promoter-TSS (NM_001142523) | −19 | NM_007199 | 11213 | ENSG00000090376 | IRAK3 |
| chr11 | 66582937 | promoter-TSS (NM_001142523) | 43 | NM_001142523 | 11213 | ENSG00000090376 | IRAK3 |
| chr12 | 70082891 | promoter-TSS (NM_152439) | 66 | NR_104220 | 144453 | ENSG00000127325 | BEST3 |
| chr12 | 70082956 | promoter-TSS (NM_152439) | 1 | NR_104220 | 144453 | ENSG00000127325 | BEST3 |
| chr12 | 70087752 | intron (NM_001282615, intron 1 of 2) | −4795 | NM_152439 | 144453 | ENSG00000127325 | BEST3 |
| chr12 | 70092965 | promoter-TSS (NM_0012X2616) | 18 | NM_032735 | 144453 | ENSG00000127325 | BEST3 |
| chr12 | 70093015 | promoter-TSS (NM_001282614) | −32 | NM_001282615 | 144453 | ENSG00000127325 | BEST3 |
| chr12 | 70093024 | promoter-TSS (NM_001282614) | −41 | NM_001282615 | 144453 | ENSG00000127325 | BEST3 |
| chr11 | 70093031 | promoter-TSS (NM_001282614) | −48 | NM_001282615 | 144453 | ENSG00000127325 | BEST3 |
| chr11 | 70093041 | promoter-TSS (NM_001282614) | 55 | NM_001282613 | 144453 | ENSG00000127325 | BEST3 |
| chr12 | 70093044 | promoter-TSS (NM_001282614) | 52 | NM_001282613 | 144453 | ENSG00000127325 | BEST3 |
| chr12 | 70093075 | promoter-TSS (NM_001282614) | 21 | NM_001282613 | 144453 | ENSG00000127325 | BEST3 |
| chr12 | 70093096 | promoter-TSS (NM_001282614) | 0 | NM_001282613 | 144453 | ENSG00000127325 | BEST3 |
| chr12 | 70093156 | promoter-TSS (NM_001282614) | −60 | NM_001282614 | 144453 | ENSG00000127325 | BEST3 |
| chr12 | 103351364 | promoter-TSS (NM_004316) | 12 | NM_004316 | 429 | ENSG00000139352 | ASCL1 |
| chr13 | 102527544 | exon (NM_001321933, exon 3 of 6) | 41351 | NM_004115 | 2259 | ENSG00000102466 | FGF14 |
| chr13 | 102568895 | promoter-TSS (NM_004115) | 0 | NM_004115 | 2259 | ENSG00000102466 | FGF14 |
| chr13 | 103054024 | promoter-TSS (NM_175929) | 0 | NM_175929 | 2259 | ENSG00000102466 | FGF14 |
| chr14 | 21994247 | non-coding (NR_111967, exon 1 of 3) | 133 | NM_001364564 | 6297 | ENSG00000165821 | SALL2 |
| chr14 | 21994261 | non-coding (NR_111967, exon 1 of 3) | 119 | NM_001364564 | 6297 | ENSG00000165821 | SALL2 |
| chr14 | 21994534 | promoter-TSS (NR_111967) | −92 | NR_111967 | 6297 | ENSG00000165821 | SALL2 |
| chr14 | 22004936 | non-coding (NR_111966, exon 1 of 3) | 219 | NM_001291446 | 6297 | ENSG00000165821 | SALL2 |
| chr14 | 22004968 | non-coding (NR_111966, exon 1 of 3) | 187 | NM_001291446 | 6297 | ENSG00000165821 | SALL2 |
| chr14 | 22005138 | promoter-TSS (NM_001291446) | 17 | NM_001291446 | 6297 | ENSG00000165821 | SALL2 |
| chr14 | 22005250 | promoter-TSS (NR_111966) | 0 | NR_111966 | 6297 | ENSG00000165821 | SALL2 |
| chr14 | 51448929 | intron (NM_015163, intron 7 of 9) | −37828 | NM_001163940 | 5836 | ENSG00000100504 | PYGL |
| chr14 | 51453070 | intron (NM_015163, intron 7 of 9) | −41969 | NM_001163940 | 5836 | ENSG00000100504 | PYGL |
| chr14 | 51454994 | intron (NM_015163, intron 7 of 9) | −43893 | NM_001163940 | 5836 | ENSG00000100504 | PYGL |
| chr14 | 51561694 | 5′ UTR (NM_015163, exon 1 of 10) | 628 | NM_015163 | 114088 | ENSG00000100505 | TRIM9 |
| chr14 | 51561947 | 5′ UTR (NM_015163, exon 1 of 10) | 375 | NM_015163 | 114088 | ENSG00000100505 | TRIM9 |
| chr14 | 51562679 | promoter-TSS (NM_015163) | −357 | NM_052978 | 114088 | ENSG00000100505 | TRIM9 |
| chr14 | 75988668 | promoter-TSS (NM_006399) | −44 | NM_006399 | 10538 | ENSG00000156127 | BATF |
| chr14 | 75988698 | promoter-TSS (NM_006399) | −14 | NM_006399 | 10538 | ENSG00000156127 | BATF |
| chr14 | 75988803 | promoter-TSS (NM_006399) | 91 | NM_006399 | 10538 | ENSG00000156127 | BATF |
| chr14 | 93389325 | promoter-TSS (NM_001275) | −70 | NM_001301690 | 1113 | ENSG00000100604 | CHGA |
| chr14 | 93389402 | promoter-TSS (NM_001275) | 7 | NM_001275 | 1113 | ENSG00000100604 | CHGA |
| chr14 | 93389421 | promoter-TSS (NM_001275) | 26 | NM_001275 | 1113 | ENSG00000100604 | CHGA |
| chr14 | 93389608 | exon (NM_001275, exon 1 of 8) | 213 | NM_001275 | 1113 | ENSG00000100604 | CHGA |
| chr14 | 93392346 | intron (NM_001301690, intron 2 of 6) | 2951 | NM_001275 | 1113 | ENSG00000100604 | CHGA |
| chr14 | 93397922 | exon (NM_001275, exon 6 of 8) | 8527 | NM_001275 | 1113 | ENSG00000100604 | CHGA |
| chr15 | 74465912 | promoter-TSS (NM_005545) | −39 | NM_005545 | 3671 | ENSG00000129009 | ISLR |
| chr15 | 74466371 | promoter-TSS (NM_201526) | −417 | NM_201526 | 3671 | ENSG00000129009 | ISLR |
| chr15 | 74466644 | promoter-TSS (NM_201526) | −144 | NM_201526 | 3671 | ENSG00000129009 | ISLR |
| chr15 | 74466742 | promoter-TSS (NM_201526) | −46 | NM_201526 | 3671 | ENSG00000129009 | ISLR |
| chr15 | 77285600 | Intergenic | −1321 | NM_001321137 | 9051 | ENSG00000140368 | PSTPIP1 |
| chr15 | 77286921 | promoter-TSS (NM_001321136) | 0 | NM_001321137 | 9051 | ENSG00000140368 | PSTPIP1 |
| chr15 | 77287326 | promoter-TSS (NM_001321136) | −41 | NM_001321136 | 9051 | ENSG00000140368 | PSTPIP1 |
| chr15 | 77287363 | promoter-TSS (NM_001321136) | −4 | NM_001321136 | 9051 | ENSG00000140368 | PSTPIP1 |
| chr15 | 77287365 | promoter-TSS (NM_001321136) | −2 | NM_001321136 | 9051 | ENSG00000140368 | PSTPIP1 |
| chr15 | 77287367 | promoter-TSS (NM_001321136) | 0 | NM_001321136 | 9051 | ENSG00000140368 | PSTPIP1 |
| chr15 | 77287372 | promoter-TSS (NM_001321136) | 5 | NR_135552 | 9051 | ENSG00000140368 | PSTPIP1 |
| chr15 | 77287413 | promoter-TSS (NM_001321136) | 46 | NR_135552 | 9051 | ENSG00000140368 | PSTPIP1 |
| chr15 | 77287603 | non-coding (NR_135552, exon 1 of 12) | 236 | NR_135552 | 9051 | ENSG00000140368 | PSTPIP1 |
| chr15 | 77287626 | non-coding (NR_135552, exon 1 of 12) | 259 | NR_135552 | 9051 | ENSG00000140368 | PSTPIP1 |
| chr15 | 77287653 | exon (NM_001321137, exon 2 of 16) | 286 | NR_135552 | 9051 | ENSG00000140368 | PSTPIP1 |
| chr15 | 77287660 | exon (NM_001321137, exon 2 of 16) | 293 | NR_135552 | 9051 | ENSG00000140368 | PSTPIP1 |
| chr15 | 77287815 | exon (NM_001321135, exon 1 of 14) | 448 | NR_135552 | 9051 | ENSG00000140368 | PSTPIP1 |
| chr15 | 77308052 | intron (NM_001321136, intron 2 of 15) | 20685 | NR_135552 | 9051 | ENSG00000140368 | PSTPIP1 |
| chr15 | 77310699 | exon (NM_001321136, exon 4 of 16) | 23332 | NR_135552 | 9051 | ENSG00000140368 | PSTPIP1 |
| chr15 | 77319966 | intron (NR_135552, intron 5 of 11) | 32599 | NR_135552 | 9051 | ENSG00000140368 | PSTPIP1 |
| chr15 | 77320584 | intron (NR_135552, intron 6 of 11) | 33217 | NR_135552 | 9051 | ENSG00000140368 | PSTPIP1 |
| chr15 | 77324558 | exon (NM_001321136, exon 12 of 16) | 37191 | NR_135552 | 9051 | ENSG00000140368 | PSTPIP1 |
| chr15 | 77327935 | intron (NR_135552, intron 10 of 11) | 35416 | NM_001168412 | 10099 | ENSG00000140391 | TSPAN3 |
| chr16 | 29674500 | promoter-TSS (NM_003123) | 32 | NM_003123 | 6693 | ENSG00000197471 | SPN |
| chr16 | 29690229 | promoter-TSS (NR_134536) | 0 | NM_001318250 | 23475 | ENSG00000103485 | QPRT |
| chr16 | 29690258 | promoter-TSS (NR_134536) | 29 | NM_001318249 | 23475 | ENSG00000103485 | QPRT |
| chr16 | 29690403 | promoter-TSS (NR_134536) | 2 | NR_134536 | 23475 | ENSG00000103485 | QPRT |
| chr16 | 29707018 | intron (NM_014298, intron 2 of 3) | 16617 | NR_134536 | 23475 | ENSG00000103485 | QPRT |
| chr16 | 31146861 | promoter-TSS (NM_002773) | 87 | NM_002773 | 5652 | ENSG00000052344 | PRSS8 |
| chr16 | 31146924 | promoter-TSS (NM_002773) | 24 | NM_002773 | 5652 | ENSG00000052344 | PRSS8 |
| chr16 | 31146948 | promoter-TSS (NM_002773) | 0 | NM_002773 | 5652 | ENSG00000052344 | PRSS8 |
| chr16 | 31146983 | promoter-TSS (NM_002773) | −35 | NM_002773 | 5652 | ENSG00000052344 | PRSS8 |
| chr16 | 74710849 | AluY\|SINE\|Alu | −10174 | NM_001370536 | 55159 | ENSG00000168411 | RFWD3 |
| chr16 | 74712211 | intron (NM_152649, intron 7 of 10) | −11536 | NM_001370536 | 55159 | ENSG00000168411 | RFWD3 |
| chr16 | 74712746 | exon (NM_152649, exon 7 of 11) | −12071 | NM_001370536 | 55159 | ENSG00000168411 | RFWD3 |
| chr16 | 74725267 | intron (NM_152649, intron 3 of 10) | 9393 | NM_152649 | 197259 | ENSG00000168404 | MLKL |
| chr16 | 74734588 | promoter-TSS (NM_001142497) | 72 | NM_152649 | 197259 | ENSG00000168404 | MLKL |
| chr16 | 74734607 | promoter-TSS (NM_001142497) | 53 | NM_152649 | 197259 | ENSG00000168404 | MLKL |
| chr16 | 74734712 | promoter-TSS (NM_001142497) | −52 | NM_001142497 | 197259 | ENSG00000168404 | MLKL |
| chr16 | 74734758 | promoter-TSS (NM_001142497) | −98 | NM_001142497 | 197259 | ENSG00000168404 | MLKL |
| chr16 | 88713133 | exon (NM_000101, exon 4 of 6) | 4228 | NM_000101 | 1535 | ENSG00000051523 | CYBA |
| chr16 | 88716622 | intron (NM_000101, intron 1 of 5) | 739 | NM_000101 | 1535 | ENSG00000051523 | CYBA |
| chr16 | 88717296 | promoter-TSS (NM_000101) | 65 | NM_000101 | 1535 | ENSG00000051523 | CYBA |
| chr16 | 88717323 | promoter-TSS (NM_000101) | 38 | NM_000101 | 1535 | ENSG00000051523 | CYBA |
| chr16 | 88717338 | promoter-TSS (NM_000101) | 23 | NM_000101 | 1535 | ENSG00000051523 | CYBA |
| chr16 | 88717339 | promoter-TSS (NM_000101) | 22 | NM_000101 | 1535 | ENSG00000051523 | CYBA |
| chr16 | 88717343 | promoter-TSS (NM_000101) | 18 | NM_000101 | 1535 | ENSG00000051523 | CYBA |
| chr16 | 88717349 | promoter-TSS (NM_000101) | 12 | NM_000101 | 1535 | ENSG00000051523 | CYBA |
| chr16 | 88717362 | promoter-TSS (NM_000101) | −1 | NM_000101 | 1535 | ENSG00000051523 | CYBA |
| chr16 | 88717460 | promoter-TSS (NM_000101) | −99 | NM_000101 | 1535 | ENSG00000051523 | CYBA |
| chr17 | 1665153 | promoter-TSS (NM_002615) | 34 | NM_001329904 | 5176 | ENSG00000132386 | SERPINF1 |
| chr17 | 1665223 | promoter-TSS (NM_002615) | −31 | NM_002615 | 5176 | ENSG00000132386 | SERPINF1 |
| chr17 | 1665255 | promoter-TSS (NM_002615) | 1 | NM_002615 | 5176 | ENSG00000132386 | SERPINF1 |
| chr17 | 1665262 | promoter-TSS (NM_002615) | 8 | NM_002615 | 5176 | ENSG00000132386 | SERPINF1 |
| chr17 | 1665306 | promoter-TSS (NM_002615) | 52 | NM_002615 | 5176 | ENSG00000132386 | SERPINF1 |
| chr17 | 1666023 | promoter-TSS (NM_001329903) | 25 | NM_001329903 | 5176 | ENSG00000132386 | SERPINF1 |
| chr17 | 1669468 | AluSx1\|SINE\|Alu | 3470 | NM_001329903 | 5176 | ENSG00000132386 | SERPINF1 |
| chr17 | 1673720 | AluSx1\|SINE\|Alu | −1145 | NM_001329905 | 5176 | ENSG00000132386 | SERPINF1 |
| chr17 | 1674913 | promoter-TSS (NM_001329905) | 48 | NM_001329905 | 5176 | ENSG00000132386 | SERPINF1 |
| chr17 | 1679644 | intron (NM_001329903, intron 6 of 7) | 4779 | NM_001329905 | 5176 | ENSG00000132386 | SERPINF1 |
| chr17 | 27283357 | exon (NM_178860, exon 15 of 17) | −4921 | NM_020889 | 57649 | ENSG00000109118 | PHF12 |
| chr17 | 27284731 | MIR\|SINE\|MIR | −6295 | NM_020889 | 57649 | ENSG00000109118 | PHF12 |
| chr17 | 27287891 | exon (NM_178860, exon 6 of 17) | −9455 | NM_020889 | 57649 | ENSG00000109118 | PHF12 |
| chr17 | 27308510 | exon (NM_178860, exon 2 of 17) | 24441 | NM_001098635 | 124925 | ENSG00000063015 | SEZ6 |
| chr17 | 27308790 | exon (NM_178860, exon 2 of 17) | 24161 | NM_001098635 | 124925 | ENSG00000063015 | SEZ6 |
| chr17 | 27332831 | 5′ UTR (NM_178860, exon 1 of 17) | 120 | NM_001098635 | 124925 | ENSG00000063015 | SEZ6 |
| chr17 | 27332981 | promoter-TSS (NM_178860) | −30 | NM_178860 | 124925 | ENSG00000063015 | SEZ6 |
| chr17 | 27333063 | promoter-TSS (NM_178860) | −112 | NM_178860 | 124925 | ENSG00000063015 | SEZ6 |
| chr17 | 27333216 | promoter-TSS (NM_178860) | 142 | NM_001290202 | 124925 | ENSG00000063015 | SEZ6 |
| chr17 | 27333358 | promoter-TSS (NM_178860) | 0 | NM_001290202 | 124925 | ENSG00000063015 | SEZ6 |
| chr17 | 62007774 | intron (NM_001039933, intron 2 of 5) | 1817 | NM_000626 | 974 | ENSG00000007312 | CD79B |
| chr17 | 62009521 | promoter-TSS (NM_000626) | 70 | NM_000626 | 974 | ENSG00000007312 | CD79B |
| chr17 | 62009583 | promoter-TSS (NM_000626) | 8 | NM_000626 | 974 | ENSG00000007312 | CD79B |
| chr17 | 62009591 | promoter-TSS (NM_000626) | 0 | NM_000626 | 974 | ENSG00000007312 | CD79B |
| chr17 | 62009596 | promoter-TSS (NM_000626) | −5 | NM_001039933 | 974 | ENSG00000007312 | CD79B |
| chr17 | 62009614 | promoter-TSS (NM_000626) | −23 | NM_001039933 | 974 | ENSG00000007312 | CD79B |
| chr17 | 76970778 | exon (NM_005567, exon 4 of 6) | 5127 | NM_005567 | 3959 | ENSG00000108679 | LGALS3BP |
| chr17 | 76970979 | intron (NM_005567, intron 3 of 5) | 4926 | NM_005567 | 3959 | ENSG00000108679 | LGALS3BP |
| chr17 | 76972138 | exon (NM_005567, exon 3 of 6) | 3767 | NM_005567 | 3959 | ENSG00000108679 | LGALS3BP |
| chr17 | 76973198 | intron (NM_005567, intron 1 of 5) | 2707 | NM_005567 | 3959 | ENSG00000108679 | LGALS3BP |
| chr17 | 76973305 | intron (NM_005567, intron 1 of 5) | 2600 | NM_005567 | 3959 | ENSG00000108679 | LGALS3BP |
| chr17 | 76975825 | promoter-TSS (NM_005567) | 80 | NM_005567 | 3959 | ENSG00000108679 | LGALS3BP |
| chr17 | 76975857 | promoter-TSS (NM_005567) | 48 | NM_005567 | 3959 | ENSG00000108679 | LGALS3BP |
| chr17 | 76975873 | promoter-TSS (NM_005567) | 32 | NM_005567 | 3959 | ENSG00000108679 | LGALS3BP |
| chr17 | 76975890 | promoter-TSS (NM_005567) | 15 | NM_005567 | 3959 | ENSG00000108679 | LGALS3BP |
| chr17 | 76975893 | promoter-TSS (NM_005567) | 12 | NM_005567 | 3959 | ENSG00000108679 | LGALS3BP |
| chr17 | 76975900 | promoter-TSS (NM_005567) | 5 | NM_005567 | 3959 | ENSG00000108679 | LGALS3BP |
| chr17 | 76975905 | promoter-TSS (NM_005567) | 0 | NM_005567 | 3959 | ENSG00000108679 | LGALS3BP |
| chr17 | 76975909 | promoter-TSS (NM_005567) | −4 | NM_005567 | 3959 | ENSG00000108679 | LGALS3BP |
| chr17 | 76975917 | promoter-TSS (NM_005567) | −12 | NM_005567 | 3959 | ENSG00000108679 | LGALS3BP |
| chr17 | 76975929 | promoter-TSS (NM_005567) | −24 | NM_005567 | 3959 | ENSG00000108679 | LGALS3BP |
| chr17 | 76976091 | promoter-TSS (NM_005567) | −186 | NM_005567 | 3959 | ENSG00000108679 | LGALS3BP |
| chr18 | 25616449 | promoter-TSS (NM_001308176) | 56 | NM_001308176 | 1000 | ENSG00000170558 | CDH2 |
| chr18 | 25625460 | L2c\|LINE\|L2 | −8955 | NM_001308176 | 1000 | ENSG00000170558 | CDH2 |
| chr18 | 25677093 | intron (NM_001792, intron 2 of 15) | −60588 | NM_001308176 | 1000 | ENSG00000170558 | CDH2 |
| chr18 | 25739161 | intron (NM_001792, intron 1 of 15) | 17833 | NM_001792 | 1000 | ENSG00000170558 | CDH2 |
| chr18 | 25757310 | promoter-TSS (NM_001792) | −316 | NM_001792 | 1000 | ENSG00000170558 | CDH2 |
| chr18 | 31158479 | 5′ UTR (NM_030632, exon 1 of 12) | 396 | NM_030632 | 80816 | ENSG00000141431 | ASXL3 |
| chr18 | 31158496 | exon (NM_030632, exon 1 of 12) | 413 | NM_030632 | 80816 | ENSG00000141431 | ASXL3 |
| chr18 | 31158505 | exon (NM_030632, exon 1 of 12) | 422 | NM_030632 | 80816 | ENSG00000141431 | ASXL3 |
| chr18 | 31158508 | exon (NM_030632, exon 1 of 12) | 425 | NM_030632 | 80816 | ENSG00000141431 | ASXL3 |
| chr18 | 31158530 | exon (NM_030632, exon 1 of 12) | 447 | NM_030632 | 80816 | ENSG00000141431 | ASXL3 |
| chr18 | 31158635 | intron (NM_030632, intron 1 of 11) | 552 | NM_030632 | 80816 | ENSG00000141431 | ASXL3 |
| chr18 | 31158746 | intron (NM_030632, intron 1 of 11) | 663 | NM_030632 | 80816 | ENSG00000141431 | ASXL3 |
| chr18 | 31185450 | intron (NM_030632, intron 1 of 11) | 27367 | NM_030632 | 80816 | ENSG00000141431 | ASXL3 |
| chr18 | 31185451 | intron (NM_030632, intron 1 of 11) | 27368 | NM_030632 | 80816 | ENSG00000141431 | ASXL3 |
| chr18 | 31185458 | intron (NM_030632, intron 1 of 11) | 27375 | NM_030632 | 80816 | ENSG00000141431 | ASXL3 |
| chr18 | 31185463 | intron (NM_030632, intron 1 of 11) | 27380 | NM_030632 | 80816 | ENSG00000141431 | ASXL3 |
| chr18 | 31185472 | intron (NM_030632, intron 1 of 11) | 27389 | NM_030632 | 80816 | ENSG00000141431 | ASXL3 |
| chr18 | 31185490 | intron (NM_030632, intron 1 of 11) | 27407 | NM_030632 | 80816 | ENSG00000141431 | ASXL3 |
| chr18 | 31187471 | exon (NM_030632, exon 2 of 12) | 29388 | NM_030632 | 80816 | ENSG00000141431 | ASXL3 |
| chr19 | 39989435 | promoter-TSS (NM_010941) | −52 | NM_203486 | 10683 | ENSG00000090932 | DLL3 |
| chr19 | 39989507 | promoter-TSS (NM_016941) | 20 | NM_016941 | 10683 | ENSG00000090932 | DLL3 |
| chr19 | 39989508 | promoter-TSS (NM_016941) | 21 | NM_016941 | 10683 | ENSG00000090932 | DLL3 |
| chr19 | 46916741 | promoter-TSS (NM_032040) | −20 | NM_032040 | 83987 | ENSG00000169515 | CCDC8 |
| chr19 | 51358071 | promoter-TSS (NM_001030048) | 0 | NM_001030047 | 354 | ENSG00000142515 | KLK3 |
| chr19 | 51358077 | promoter-TSS (NM_001030048) | 6 | NM_001030048 | 354 | ENSG00000142515 | KLK3 |
| chr19 | 51358086 | promoter-TSS (NM_001030048) | 15 | NM_001030048 | 354 | ENSG00000142515 | KLK3 |
| chr19 | 51358091 | promoter-TSS (NM_001030048) | 20 | NM_001030048 | 354 | ENSG00000142515 | KLK3 |
| chr19 | 51358105 | promoter-TSS (NM_001030048) | 34 | NM_001030048 | 354 | ENSG00000142515 | KLK3 |
| chr19 | 51358112 | promoter-TSS (NM_001030048) | 41 | NM_001030048 | 354 | ENSG00000142515 | KLK3 |
| chr19 | 51358128 | promoter-TSS (NM_001030048) | 57 | NM_001030048 | 354 | ENSG00000142515 | KLK3 |
| chr19 | 51360547 | intron (NM_001030048, intron 2 of 4) | 2476 | NM_001030048 | 354 | ENSG00000142515 | KLK3 |
| chr20 | 3210322 | exon (NM_001174089, exon 14 of 20) | 7951 | NM_032034 | 83959 | ENSG00000088836 | SLC4A11 |
| chr20 | 3211106 | exon (NM_001174089, exon 12 of 20) | 7167 | NM_032034 | 83959 | ENSG00000088836 | SLC4A11 |
| chr20 | 3218273 | promoter-TSS (NM_032034) | 0 | NM_032034 | 83959 | ENSG00000088836 | SLC4A11 |
| chr20 | 3218712 | promoter-TSS (NM_032034) | 23 | NM_001174090 | 83959 | ENSG00000088836 | SLC4A11 |
| chr20 | 3219259 | promoter-TSS (NM_032034) | 477 | NM_001363745 | 83959 | ENSG00000088836 | SLC4A11 |
| chr20 | 3219736 | promoter-TSS (NM_001174089) | 0 | NM_001363745 | 83959 | ENSG00000088836 | SLC4A11 |
| chr20 | 5891976 | promoter-TSS (NM_001819) | −80 | NM_001819 | 1114 | ENSG00000089199 | CHGB |
| chr20 | 5892076 | promoter-TSS (NM_001819) | 20 | NM_001819 | 1114 | ENSG00000089199 | CHGB |
| chr20 | 5896044 | intron (NM_001819, intron 1 of 4) | 3988 | NM_001819 | 1114 | ENSG00000089199 | CHGB |
| chr20 | 20348665 | promoter-TSS (NM_002196) | 17 | NM_002196 | 3642 | ENSG00000173404 | INSM1 |
| chr20 | 22565001 | promoter-TSS (NM_021784) | 0 | NM_021784 | 3170 | ENSG00000125798 | FOXA2 |
| chr20 | 22565993 | promoter-TSS (NM_153675) | 35 | NM_153675 | 3170 | ENSG00000125798 | FOXA2 |
| chr21 | 42839929 | intron (NM_005656, intron 12 of 13) | 36052 | NM_001282920 | 4599 | ENSG00000157601 | MX1 |
| chr21 | 42840664 | intron (NM_005656, intron 11 of 13) | 36787 | NM_001282920 | 4599 | ENSG00000157601 | MX1 |
| chr21 | 42878431 | intron (NM_005656, intron 1 of 13) | 1461 | NM_001135099 | 7113 | ENSG00000184012 | TMPRSS2 |
| chr21 | 42879809 | promoter-TSS (NM_001135099) | 83 | NM_001135099 | 7113 | ENSG00000184012 | TMPRSS2 |
| chr21 | 42879892 | promoter-TSS (NM_001135099) | 0 | NM_001135099 | 7113 | ENSG00000184012 | TMPRSS2 |
| chr21 | 42879919 | promoter-TSS (NM_001135099) | −27 | NM_001135099 | 7113 | ENSG00000184012 | TMPRSS2 |
| chr21 | 42879941 | promoter-TSS (NM_001135099) | 44 | NM_005656 | 7113 | ENSG00000184012 | TMPRSS2 |
| chr21 | 42879985 | promoter-TSS (NM_001135099) | 0 | NM_005656 | 7113 | ENSG00000184012 | TMPRSS2 |
| chr21 | 42879986 | promoter-TSS (NM_001135099) | −1 | NM_005656 | 7113 | ENSG00000184012 | TMPRSS2 |
| chr21 | 42902925 | Intergenic | −22940 | NM_005656 | 7113 | ENSG00000184012 | TMPRSS2 |
| chr21 | 42902943 | Intergenic | −22958 | NM_005656 | 7113 | ENSG00000184012 | TMPRSS2 |
| chrX | 49056535 | promoter-TSS (NM_003179) | 26 | NM_003179 | 6855 | ENSG00000102003 | SYP |
| chrX | 49056552 | promoter-TSS (NM_003179) | 9 | NM_003179 | 6855 | ENSG00000102003 | SYP |
| chrX | 49056554 | promoter-TSS (NM_003179) | 7 | NM_003179 | 6855 | ENSG00000102003 | SYP |
| chrX | 49056558 | promoter-TSS (NM_003179) | 3 | NM_003179 | 6855 | ENSG00000102003 | SYP |
| chrX | 49056618 | promoter-TSS (NM_003179) | −57 | NM_003179 | 6855 | ENSG00000102003 | SYP |
| chrX | 66764365 | promoter-TSS (NM_001348064) | 0 | NM_001011645 | 367 | ENSG00000169083 | AR |
| chrX | 66764562 | 5′ UTR (NM_001348064, exon 1 of 3) | 197 | NM_001348061 | 367 | ENSG00000169083 | AR |
| chrX | 66764886 | 5′ UTR (NM_001348064, exon 1 of 3) | 521 | NM_001348061 | 367 | ENSG00000169083 | AR |
| chrX | 66788631 | 5′ UTR (NM_001011645, exon 2 of 9) | 24266 | NM_001348061 | 367 | ENSG00000169083 | AR |
| chr1 | 202039153 | AluSx1\|SINE\|Alu | −52892 | NM_004767 | 9283 | ENSG00000170075 | GPR37L1 |
| chr1 | 241206098 | AluSx\|SINE\|Alu | −89474 | NR_036069 | 100422856 | ENSG00000265831 | MIR3123 |
| chr2 | 89530057 | Intergenic | 418173 | NR_039635 | 100616399 | ENSG00000265510 | MIR4436A |
| chr3 | 193490472 | Intergenic | 71326 | NR_146718 | 105374285 | | LINC02038 |
| chr5 | 1548487 | Intergenic | −24412 | NM_024830 | 79888 | ENSG00000153395 | LPCAT1 |
| chr5 | 36521979 | Intergenic | −84478 | NM_001166696 | 6507 | ENSG00000079215 | SLC1A3 |
| chr5 | 39390555 | exon (NM_001244871, exon 5 of 14) | −25958 | NM_001737 | 735 | ENSG00000113600 | C9 |
| chr5 | 71146894 | LSU-rRNA_Hsa\|rRNA\|rRNA | 131792 | NM_004291 | 9607 | ENSG00000164326 | CARTPT |
| chr5 | 142194123 | intron (NM_001349547, intron 1 of 21) | 43331 | NM_001349547 | 23092 | ENSG00000145819 | ARHGAP26 |
| chr5 | 168573593 | intron (NM_001271946, intron 4 of 35) | 117105 | NR_030311 | 693170 | ENSG00000207619 | MIR585 |
| chr7 | 2006579 | L1MEf\|LINE\|L1 | −26372 | NM_001304525 | 8379 | ENSG00000002822 | MAD1L1 |
| chr7 | 7799018 | intron (NM_001302350, intron 3 of 4) | −40780 | NM_002947 | 6119 | ENSG00000106399 | RPA3 |
| chr7 | 127841628 | CpG | −6297 | NR_029596 | 406917 | ENSG00000207705 | MIR129-1 |
| chr7 | 129912398 | LTR88c\|LTR\|Gypsy? | 5695 | NM_001869 | 1358 | ENSG00000158516 | CPA2 |
| chr8 | 53499560 | Intergenic | −21570 | NM_207413 | 389658 | ENSG00000196711 | ALKAL1 |
| chr8 | 61990390 | Intergenic | −110083 | NR_034003 | 100130298 | | LOC100130298 |
| chr8 | 69087227 | L2c\|LINE\|L2 | −155381 | NM_001349478 | 116328 | ENSG00000165084 | C8orf34 |
| chr8 | 73479990 | exon (NM_004770, exon 2 of 3) | 30530 | NM_004770 | 9312 | ENSG00000182674 | KCNB2 |
| chr8 | 73849054 | exon (NM_004770, exon 3 of 3) | −72045 | NM_017489 | 7013 | ENSG00000147601 | TERF1 |
| chr8 | 133070961 | MIRb\|SINE\|MIR | 668 | NM_001080399 | 729330 | ENSG00000253117 | OC90 |
| chr8 | 140727998 | Intergenic | −12632 | NM_001282534 | 51305 | ENSG00000169427 | KCNK9 |
| chr8 | 141458926 | MIRb\|SINE\|MIR | 8917 | NM_001160372 | 83696 | ENSG00000167632 | TRAPPC9 |
| chr8 | 141994084 | 5′ UTR (NM_001352698, exon 2 of 36): | 17019 | NM_001352710 | 5747 | ENSG00000169398 | PTK2 |
| chr8 | 142367954 | exon (NM_005293, exon 2 of 2) | 9432 | NM_005293 | 2843 | ENSG00000204882 | GPR20 |
| chr8 | 143395944 | intron (NM_001363740, intron 8 of 13); | 88599 | NM_001366904 | 203062 | ENSG00000171045 | TSNARE1 |
| chr9 | 36748433 | Intergenic | 75163 | NR_039686 | 100616289 | ENSG00000266255 | MIR4475 |
| chr9 | 38437314 | Intergenic | −12863 | NM_001007563 | 347252 | ENSG00000137142 | IGFBPL1 |
| chr9 | 93792388 | LTR82B\|LTR\|ERVL | 45026 | NR_033912 | 100129316 | | LOC100129316 |
| chr9 | 133769512 | intron (NM_198180, intron 2 of 2) | 2687 | NM_198180 | 347148 | ENSG00000188710 | QRFP |
| chr9 | 134913164 | intron (NM_004269, intron 2 of 7) | 42090 | NM_001253881 | 9442 | ENSG00000160563 | MED27 |
| chr9 | 138719304 | exon (NM_015447, exon8 of 17) | 80088 | NM_015447 | 157922 | ENSG00000130559 | CAMSAP1 |
| chr14 | 100039425 | intron (NM_001144995, intron 1 of 5) | 31119 | NM_001144995 | 317762 | ENSG00000205476 | CCDC85C |
| chr18 | 8660098 | CpG | 47521 | NR_120598 | 100287082 | ENSG00000265962 | GACAT2 |
| chr18 | 14917016 | Intergenic | −29250 | NR_104164 | 400644 | ENSG00000266554 | LINC01443 |
| chr18 | 25442246 | Intergenic | 174359 | NM_001308176 | 1000 | ENSG00000170558 | CDH2 |
| chr18 | 47258089 | MLT1K\|LTR\|ERVL-MaLR | 81814 | NM_006111 | 10449 | ENSG00000167315 | ACAA2 |
| chr20 | 20421462 | intron (NM_020343, intron 37 of 39) | 72714 | NM_002196 | 3642 | ENSG00000173404 | INSM1 |
| chr20 | 21563921 | intron (NR_109880, intron 1 of 3) | 13259 | NR_109880 | 101929625 | | LINC01727 |
| chr21 | 32546326 | intron (NM_001353687, intron 14 of 26) | −13069 | NM_001353684 | 7074 | ENSG00000156299 | TIAM1 |
| chr21 | 46885392 | intron (NM_130444, intron 1 of 41) | 9956 | NM_130444 | 80781 | ENSG00000182871 | COL18A1 |

| | start_hg19 | Gene Alias | Gene Description | Gene Type |
|---|---|---|---|---|
| chr1 | 32237823 | BAI2 | adhesion G protein-coupled receptor B2 | protein-coding |
| chr1 | 43814069 | CDC20A\|bA276H19.3\|p55CDC | cell division cycle 20 | protein-coding |
| chr1 | 151345023 | EHMTO\|HEL-S-134P\|LPSB\|MTO\|SBP56\|SP56\|hSBP | selenium binding protein 1 | protein-coding |
| chr1 | 151810787 | — | C2CD4D and THEM5 antisense RNA 1 | ncRNA |
| chr2 | 19561227 | ODD | odd-skipped related transcription factor 1 | protein-coding |
| chr2 | 63283706 | — | orthodenticle homeobox 1 | protein-coding |
| chr2 | 177030050 | HOX1D\|HOX4\|HOX4A\|Hox-4.1 | homeobox D3 | protein-coding |
| chr2 | 201450223 | AO\|AOH1 | aldehyde oxidase 1 | protein-coding |
| chr3 | 50378091 | 123F2\|NORE2A\|RASSF1A\|RDA32\|REH3P21 | Ras association domain family member 1 | protein-coding |
| chr3 | 122296269 | ARTD7\|BAL3\|pART7 | poly(ADP-ribose) polymerase family | protein-coding |
| | | | member 15 | |
| chr3 | 124860516 | — | microRNA 5092 | ncRNA |
| chr5 | 958708 | — | uncharacterized LOC100506688 | ncRNA |
| chr5 | 3187966 | — | long intergenic non-protein coding RNA | ncRNA |
| | | | 1377 | |
| chr5 | 180230667 | GLCNAC-TI\|GLCT1\|GLYT1\|GNT-1\|GNT-I\|GnTI\|MGAT | mannosyl (alpha-1,3-)-glycoprotein beta- | protein-coding |
| | | | 1,2-N-acetylglucosaminyltransferase | |
| chr6 | 13014771 | EIEE70\|RPEL\|RPEL1\|dJ257A7.2 | phosphatase and actin regulator 1 | protein-coding |
| chr6 | 15505245 | BLOC1S8\|DBND\|HPS7\|My031\|SDY | dystrobrevin binding protein 1 | protein-coding |
| chr6 | 26271093 | H3/h\|H3FH | histone cluster 1 H3 family member g | protein-coding |
| chr7 | 73245339 | CPE−R\|CPER\|CPETR\|CPETR1\|WBSCR8\|hCPE−R | claudin 4 | protein-coding |
| chr7 | 129425230 | MIRN183\|miR-183\|mRNA183 | microRNA 183 | ncRNA |
| chr8 | 56852048 | — | small nucleolar RNA, H/ACAbox 1B | snoRNA |
| chr8 | 99960398 | — | odd-skipped related transciption factor 2 | protein-coding |
| chr8 | 143625497 | Arg3.1\|hArc | activity regulated cytoskeleton associated | protein-coding |
| | | | protein | |
| chr8 | 145104006 | MA-nSMase | sphingomyelin phosphodiesterase 5 | pseudo |
| chr11 | 6297648 | IPFK2\|PFK2\|iPFK-2 | 6-phosphofructo-2-kinase/fructose-2,6- | protein-coding |
| | | | biphosphatase 3 | |
| chr11 | 22541895 | — | uncharacterized LOC100130992 | ncRNA |
| chr11 | 393838 | — | plakophilin 3 | protein-coding |
| chr11 | 47416258 | mir-4487 | microRNA 4487 | ncRNA |
| chr11 | 63974672 | KIND3\|MIG-2\|MIG2B\|UNC112C\|URP2\|URP2SF | fermitin family member 3 | protein-coding |
| chr11 | 165408329 | SPA1 | signal-induced proliferation-associated 1 | protein-coding |
| chr11 | 67204996 | CD45-AP\|LPAP | protein tyrosine phosphatase receptor type | protein-coding |
| | | | C associated protein | |
| chr11 | 69469526 | BCL1\|D11S287E\|PRAD1\|U21B31 | cvclin D1 | protein-coding |
| chr12 | 7060087 | HCP\|HCPH\|HPTP1C\|PTP-1C\|SH-PTP1\|SHP-1\|SHP- | protein tyrosine phosphatase non-receptor | protein-coding |
| | | 1L\|SHP1 | type 6 | |
| chr12 | 53298578 | CARD2\|CK-8\|CK8\|CYK8\|K2C8\|K8\|KO | keratin 8 | protein-coding |
| chr12 | 95941769 | — | ubiquitin specific peptidase 44 | protein-coding |
| chr12 | 107038917 | — | uncharacterized LOC100505978 | ncRNA |
| chr12 | 124773568 | CFM2\|FAM101A | refilin A | protein-coding |
| chr12 | 124950569 | CTG26\|N-CoR2\|SMAP270\|SMRT\|SMRTE\|SMRTE- | nuclear receptor corepressor 2 | protein-coding |
| | | tau\|TNRC14\|TRAC\|TRAC-1\|TRAC1 | | |
| chr16 | 1128589 | — | SSTR5 antisense RNA 1 | ncRNA |
| chr17 | 26732020 | G21\|HCP1\|PCFT | solute carrier family 46 member 1 | protein-coding |
| chr17 | 41363402 | — | transmembrane protein 106A | protein-coding |
| chr17 | 43318510 | — | MAP3K14 antisense RNA 1 | ncRNA |
| chr17 | 79251155 | PP1744 | solute carrier family 38 member 10 | protein-coding |
| chr17 | 80358729 | HEXDC | hexosaminidase D | protein-coding |
| chr17 | 180847037 | ZFP750 | zinc finger protein 750 | protein-coding |
| chr19 | 2085186 | MOB-LAK\|MOB1C\|MOBKL2A\|moblak | MOB kinase activator 3A | protein-coding |
| chr19 | 2588141 | hsa-mir-7850 | microRNA 7850 | ncRNA |
| chr19 | 3179264 | EDG6\|LPC1\|S1P4\|SLP4 | sphingosine-1-phosphate receptor 4 | protein-coding |
| chr19 | 10735906 | CTL2\|PP1292 | solute carrier family 44 member 2 | protein-coding |
| chr19 | 15568260 | — | RAS protein activator like 3 | protein-coding |
| chr19 | 18872937 | EDM1\|EPD1\|MED\|PSACH\|THBS5\|TSP5 | cartilage oligomeric matrix protein | protein-coding |
| chr19 | 35940325 | FFA2R\|GPR43 | free fatty acid receptor 2 | protein-coding |
| chr19 | 42703509 | FLAME−3\|FLAME3 | death effector domain containing 2 | protein-coding |
| chr22 | 48027541 | — | uncharacterized LOC284930 | ncRNA |
| chr1 | 2036183 | PKC-ZETA\|PKC2 | protein kinase C zeta | protein-coding |
| chr1 | 2309984 | — | MORN repeat containing 1 | protein-coding |
| chr1 | 2379650 | PLC-L4\|PLC-eta2\|PLCL4\|PLCeta2 | phospholipase C eta 2 | protein-coding |
| chr1 | 9887711 | ALC-ALPHA\|CDHR12\|CST- | calsyntenin 1 | protein-coding |
| | | 1\|CSTN1\|PIK3CD\|XB31alpha\|alcalpha1\|alcalpha2 | | |
| chr1 | 119870187 | — | long intergenic non-protein coding RNA | ncRNA |
| | | | 1780 | |
| chr1 | 150122937 | CKIP-1\|CKIP1\|JBP\|OC120 | pleckstrin homology domain containing O1 | protein-coding |
| chr1 | 155034908 | EFL4\|EPLG4\|LERK4 | ephrin A4 | protein-coding |
| chr1 | 156215318 | PRdelta | progestin and adipoQ receptor family | protein-coding |
| | | | member 6 | |
| chr1 | 205631878 | IPCA-2\|IPCA-6\|IPCA- | solute carrier family 45 member 3 | protein-coding |
| | | 8\|IPCA6\|PCANAP2\|PCANAP6\|PCANAP8\|PRST | | |
| chr1 | 234666987 | — | long intergenic non-protein coding RNA | ncRNA |
| | | | 1354 | |
| chr1 | 234908126 | — | uncharacterized LOC100130249 | ncRNA |
| chr1 | 235805303 | — | G protein subunit gamma 4 | protein-coding |
| chr1 | 246939116 | — | long intergenic non-protein coding RNA | ncRNA |
| | | | 1341 | |
| chr2 | 3281230 | EIPR-1\|TSSC1 | EARP complex and GARP complex | protein-coding |
| | | | interacting protein 1 | |
| chr2 | 10567426 | ACA67B | small nucleolar RNA, H/ACA box 80B | snoRNA |
| chr2 | 11796686 | NTR2 | neurotensin receptor 2 | protein-coding |
| chr2 | 45237589 | — | SIX homeobox 2 | protein-coding |
| chr2 | 121432974 | | | |
| chr2 | 172952783 | — | distal-less homeobox 1 | protein-coding |
| chr2 | 240161519 | — | uncharacterized LOC85009 | ncRNA |
| chr3 | 134125473 | — | microRNA 4788 | ncRNA |
| chr3 | 140813669 | SSB-4\|SSB4 | splA/ryanodine receptor domain and SOCS | protein-coding |
| | | | box containing 4 | |
| chr3 | 160167217 | IFT80-L | TRIM59-1FT80 protein | ncRNA |
| chr3 | 183891828 | AP50\|CLAPM1\|mu2 | adaptor related protein complex 2 subunit | protein-coding |
| | | | mu 1 | |
| chr4 | 981385 | IDA\|MPS1 | iduronidase alpha-L- | protein-coding |
| chr4 | 1205814 | PCAT10 | CTBP1 antisense RNA | ncRNA |
| chr4 | 7228168 | — | sortilin related VPS10 domain containing | protein-coding |
| | | | receptor 2 | |
| chr5 | 92938049 | mir-2277 | microRNA 2277 | ncRNA |
| chr5 | 134827036 | — | TIFA inhibitor | protein-coding |
| chr5 | 147162061 | JAMIP2\|NECC1 | janus kinase and microtubule interacting | protein-coding |
| | | | protein 2 | |
| chr5 | 179588340 | — | RasGEF domain family member 1C | protein-coding |
| chr6 | 40567217 | FIGLER2\|KIAA1246\|SALM1 | leucine rich repeat and fibronectin type III | protein-coding |
| | | | domain containing 2 | |
| chr6 | 170449317 | — | uncharacterized LOC102724511 | ncRNA |
| chr6 | 170578492 | — | long intergenic non-protein coding RNA | ncRNA |
| | | | 1624 | |
| chr7 | 214477 | DMP-4\|DMP4\|G-CK\|GEF-CK\|RNS | FAM20C golgi associated secretory | protein-coding |
| | | | pathway kinase | |
| chr7 | 906626 | C7orf20\|CEE\|CGI-20\|TRC35 | guided entry of tail-anchored proteins factor | protein-coding |
| | | | 4 | |
| chr7 | 921951 | C7orf20\|CEE\|CGI-20\|TRC35 | guided entry of tail-anchored proteins factor | protein-coding |
| | | | 4 | |
| chr7 | 2968216 | NNX3\|RMP\|gep | G protein subunit alpha 12 | protein-coding |
| chr7 | 50628618 | AADC | dopa decarboxylase | protein-coding |
| chr7 | 129912387 | — | carboxypeptidase A2 | protein-coding |
| chr7 | 151432920 | AAKG\|AAKG2\|CMH6\|H91620p\|WPWS | protein kinase AMP-activated non-catalytic | protein-coding |
| | | | subunit gamma 2 | |
| chr8 | 73848837 | PIN2\|TRBF1\|TRF\|TRF1\|hTRF1-AS\|t-TRF1 | telomeric repeat binding factor 1 | protein-coding |
| chr8 | 93114050 | AML1- | RUNX1 partner transcriptional co-repressor | protein-coding |
| | | MTG8\|AML1T1\|CBFA2T1\|CDR\|ETO\|MTG8\|ZMYND2 | 1 | |
| chr10 | 670687 | — | uncharacterized LOC101930421 | ncRNA |
| chr10 | 123923781 | AZU-1\|ECTACC | transforming acidic coiled-coil containing | protein-coding |
| | | | protein 2 | |
| chr11 | 20625892 | GLYT-2\|GLYT2\|HKPX3\|NET1 | solute carrier family 6 member 5 | protein-coding |
| chr11 | 61449637 | C11orf11\|DAGL(ALPHA)\|DAGLALPHA\|NSDDR | diacylglycerol lipase alpha | protein-coding |
| chr11 | 62211890 | CC10\|CC16\|CCPBP\|CCSP\|UGB\|UP-1\|UP1 | secretoglobin family 1A member 1 | protein-coding |
| chr12 | 104852255 | C4ST\|C4ST-1\|C4STI\|HSA269537\|OCBMD | carbohydrate sulfotransferase 11 | protein-coding |
| chr12 | 110172108 | C12orf34 | family with sequence similarity 222 | protein-coding |
| | | | member A | |
| chr12 | 125578255 | ACSF1\|SUR-5 | acetoacetyl-CoA synthetase | protein-coding |
| chr14 | 91862713 | DAPLE\|HKRP2\|HYC1\|KIAA1509\|SCA40 | coiled-coil domain containing 88C | protein-coding |
| chr14 | 95693684 | DCR1\|Dicer\|Dicer1e\|GLOW\|HERNA\|K12H4.8- | dicer 1, ribonuclease III | protein-coding |
| | | LIKE\|MNG1\|RMSE2 | | |
| chr14 | 101157815 | C14orf70 | long intergenic non-protein coding RNA | ncRNA |
| | | | 523 | |
| chr15 | 89921572 | LINC00925 | MIR9-3 host gene | ncRNA |
| chr15 | 89959557 | — | uncharacterized LOC105371031 | ncRNA |
| chr15 | 99194331 | IGF1R-AS | IGF1R antisense imprinted non-protein | ncRNA |
| | | | coding RNA | |
| chr16 | 1195273 | CACNA1HB\|Cav3.2\|ECA6\|EIG6\|HALD4 | calcium voltage-gated channel subunit | protein-coding |
| | | | alpha1 H | |
| chr16 | 29324459 | RUNDC2C | sorting nexin 29 pseudogene 2 | pseudo |
| chr17 | 59480574 | VETD | T-box transcription factor 2 | protein-coding |
| chr17 | 60827102 | — | uncharacterized LOC105371855 | ncRNA |
| chr17 | 71744333 | — | uncharacterized LOC100134391 | protein-coding |
| chr17 | 72350254 | BTBD17A\|LGALS3BPL\|TANGO10A | BTB domain containing 17 | protein-coding |
| chr17 | 73996145 | — | cyclin dependent kinase 3 | protein-coding |
| chr17 | 76355046 | ATOD4\|CIS3\|Cish3\|SOCS-3\|SSI-3\|SSI3 | suppressor of cytokine signaling 3 | protein-coding |
| chr17 | 78079381 | LYAG | glucosidase alpha, acid | protein-coding |
| chr17 | 79223965 | C17orf89 | NADH:ubiquinone oxidoreductase complex | protein-coding |
| | | | assembly factors | |
| chr17 | 80019426 | DUS1\|PP3111 | dihydrouridine synthase 1 like | protein-coding |
| chr17 | 80052807 | FAS\|OA-519\|SDR27X1 | fatty acid synthase | protein-coding |
| chr17 | 80372675 | HEXDC | hexosaminidase D | protein-coding |
| chr17 | 80832228 | ZFP750 | zinc finger protein 750 | protein-coding |
| chr18 | 8659409 | MTCL1-AS1\|MTCL1AS1 | gastric cancer associated transcript 2 | ncRNA |
| chr18 | 13136240 | C18orf1 | low density lipoprotein receptor class A | protein-coding |
| | | | domain containing 4 | |
| chr19 | 2540807 | GADD45BETA\|MYD118 | grow th arrest and DNA damage inducible | protein-coding |
| | | | beta | |
| chr19 | 4566300 | SEM-SEMA-Y\|SEMA-VIB\|SEMAN\|semaZ | semaphorin 6B | protein-coding |
| chr19 | 17790974 | Munc13-1 | unc-13 homolog A | protein-coding |
| chr20 | 20432962 | IA-1\|IA1 | INSM transcriptional repressor 1 | protein-coding |
| chr20 | 51589516 | C20orf17\|OVC10-2\|TSH2\|ZABC2\|ZNF218 | teashirt zinc finger homeobox 2 | protein-coding |
| chr21 | 38077158 | HMC13F06\|HMC29C01\|SIM\|bHLHe15 | SIM bHLH transcription factor 2 | protein-coding |
| chr21 | 43441927 | C21orf121\|NCRNA00318\|PRED87 | ZNF295 antisense RNA 1 | ncRNA |
| chr1 | 2036220 | PKC-ZETA\|PKC2 | protein kinase C zeta | protein-coding |
| chr1 | 2036226 | PKC-ZETA\|PKC2 | protein kinase C zeta | protein-coding |
| chr1 | 2036232 | PKC-ZETA\|PKC2 | protein kinase C zeta | protein-coding |
| chr1 | 2036252 | PKC-ZETA\|PKC2 | protein kinase C zeta | protein-coding |
| chr1 | 2036395 | PKC-ZETA\|PKC2 | protein kinase C zeta | protein-coding |
| chr1 | 3697617 | Vel | small integral membrane protein 1 (Vel | protein-coding |
| | | | blood group) | |
| chr1 | 3697629 | Vel | small integral membrane protein 1 (Vel | protein-coding |
| | | | blood group) | |
| chr1 | 3697639 | Vel | small integral membrane protein 1 (Vel | protein-coding |
| | | | blood group) | |
| chr1 | 3697648 | Vel | small integral membrane protein 1 (Vel | protein-coding |
| | | | blood group) | |
| chr1 | 3697654 | Vel | small integral membrane protein 1 (Vel | protein-coding |
| | | | blood group) | |
| chr1 | 3697667 | Vel | small integral membrane protein 1 (Vel | protein-coding |
| | | | blood group) | |
| chr1 | 3697692 | Vel | small integral membrane protein 1 (Vel | protein-coding |
| | | | blood group) | |
| chr1 | 3697702 | Vel | small integral membrane protein 1 (Vel | protein-coding |
| | | | blood group) | |
| chr1 | 6171653 | AKR6A5\|HKvbeta2\|HKvbeta2.1\|HKvbeta2.2\|KCNA2B\| | potassium voltage-gated channel subfamily | protein-coding |
| | | KV-BETA-2 | A regulatory beta subunit 2 | |
| chr1 | 6171833 | AKR6A5\|HKvbeta2\|HKvbeta2.1\|HKvbeta2.2\|KCNA2B\| | potassium voltage-gated channel subfamily | protein-coding |
| | | KV-BETA-2 | A regulatory beta subunit 2 | |
| chr1 | 6171838 | AKR6A5\|HKvbeta2\|HKvbeta2.1\|HKvbeta2.2\|KCNA2B\| | potassium voltage-gated channel subfamily | protein-coding |
| | | KV-BETA-2 | A regulatory beta subunit 2 | |
| chr1 | 6171845 | AKR6A5\|HKvbeta2\|HKvbeta2.1\|HKvbeta2.2\|KCNA2B\| | potassium voltage-gated channel subfamily | protein-coding |
| | | KV-BETA-2 | A regulatory beta subunit 2 | |
| chr1 | 6171867 | AKR6A5\|HKvbeta2\|HKvbeta2.1\|HKvbeta2.2\|KCNA2B\| | potassium voltage-gated channel subfamily | protein-coding |
| | | KV-BETA-2 | A regulatory beta subunit 2 | |
| chr1 | 9501648 | — | long intergenic non-protein coding RNA | ncRNA |
| | | | 2606 | |
| chr1 | 10696021 | CAS11\|CST\|SRG\|ZNF693\|dJ734G22.1 | castor zinc finger 1 | protein-coding |
| chr1 | 10696065 | CAS11\|CST\|SRG\|ZNF693\|dJ734G22.1 | castor zinc finger 1 | protein-coding |
| chr1 | 18808323 | — | kelch domain containing 7A | protein-coding |
| chr1 | 18808343 | — | kelch domain containing 7A | protein-coding |
| chr1 | 18808439 | — | kelch domain containing 7A | protein-coding |
| chr1 | 18808469 | — | kelch domain containing 7A | protein-coding |
| chr1 | 18808546 | — | kelch domain containing 7A | protein-coding |
| chr1 | 18808583 | — | kelch domain containing 7A | protein-coding |
| chr1 | 18808621 | — | kelch domain containing 7A | protein-coding |
| chr1 | 18808832 | — | kelch domain containing 7A | protein-coding |
| chr1 | 18808880 | — | kelch domain containing 7A | protein-coding |
| chr1 | 18808929 | — | kelch domain containing 7A | protein-coding |
| chr1 | 18808938 | — | kelch domain containing 7A | protein-coding |
| chr1 | 18808967 | — | kelch domain containing 7A | protein-coding |
| chr1 | 18808991 | — | kelch domain containing 7A | protein-coding |
| chr1 | 18809009 | — | kelch domain containing 7A | protein-coding |
| chr1 | 18809028 | — | kelch domain containing 7A | protein-coding |
| chr1 | 18809041 | — | kelch domain containing 7A | protein-coding |
| chr1 | 18809052 | — | kelch domain containing 7A | protein-coding |
| chr1 | 18809058 | — | kelch domain containing 7A | protein-coding |
| chr1 | 18809064 | — | kelch domain containing 7A | protein-coding |
| chr1 | 18809102 | — | kelch domain containing 7A | protein-coding |
| chr1 | 18809112 | — | kelch domain containing 7A | protein-coding |
| chr1 | 18809126 | — | kelch domain containing 7A | protein-coding |
| chr1 | 18809494 | — | kelch domain containing 7A | protein-coding |
| chr1 | 20141125 | — | ring finger protein 186 | protein-coding |
| chr1 | 20141136 | — | ring finger protein 186 | protein-coding |
| chr1 | 20141322 | — | ring finger protein 186 | protein-coding |
| chr1 | 20141344 | — | ring finger protein 186 | protein-coding |
| chr1 | 20141404 | — | ring finger protein 186 | protein-coding |
| chr1 | 20141442 | — | ring finger protein 186 | protein-coding |
| chr1 | 20141462 | — | ring finger protein 186 | protein-coding |
| chr1 | 22195529 | — | low density lipoprotein receptor class A | protein-coding |
| | | | domain containing 2 | |
| chr1 | 32252163 | BAI2 | adhesion G protein-coupled receptor B2 | protein-coding |
| chr1 | 32252229 | BAI2 | adhesion G protein-coupled receptor B2 | protein-coding |
| chr1 | 32252239 | BAI2 | adhesion G protein-coupled receptor B2 | protein-coding |
| chr1 | 32252264 | BAI2 | adhesion G protein-coupled receptor B2 | protein-coding |
| chr1 | 32252425 | BAI2 | adhesion G protein-coupled receptor B2 | protein-coding |
| chr1 | 45103348 | — | small nucleolar RNA, C/D box 145 | snoRNA |
| chr1 | 45103390 | — | small nucleolar RNA, C/D box 145 | snoRNA |
| chr1 | 45106491 | — | small nucleolar RNA, C/D box 145 | snoRNA |
| chr1 | 156215343 | PRdelta | progestin and adipoQ receptor family | protein-coding |
| | | | member 6 | |
| chr1 | 156215371 | PRdelta | progestin and adipoQ receptor family | protein-coding |
| | | | member 6 | |
| chr1 | 156215401 | PRdelta | progestin and adipoQ receptor family | protein-coding |
| | | | member 6 | |
| chr1 | 156215416 | PRdelta | progestin and adipoQ receptor family | protein-coding |
| | | | member 6 | |
| chr1 | 156215430 | PRdelta | progestin and adipoQ receptor family | protein-coding |
| | | | member 6 | |
| chr1 | 156215445 | PRdelta | progestin and adipoQ receptor family | protein-coding |
| | | | member 6 | |
| chr1 | 156215454 | PRdelta | progestin and adipoQ receptor family | protein-coding |
| | | | member 6 | |
| chr1 | 156215491 | PRdelta | progestin and adipoQ receptor family | protein-coding |
| | | | member 6 | |
| chr1 | 156215506 | PRdelta | progestin and adipoQ receptor family | protein-coding |
| | | | member 6 | |
| chr1 | 156215520 | PRdelta | progestin and adipoQ receptor family | protein-coding |
| | | | member 6 | |
| chr1 | 156215525 | PRdelta | progestin and adipoQ receptor family | protein-coding |
| | | | member 6 | |
| chr1 | 156215530 | PRdelta | progestin and adipoQ receptor family | protein-coding |
| | | | member 6 | |
| chr1 | 156215563 | PRdelta | progestin and adipoQ receptor family | protein-coding |
| | | | member 6 | |
| chr1 | 156215590 | PRdelta | progestin and adipoQ receptor family | protein-coding |
| | | | member 6 | |
| chr1 | 156215601 | PRdelta | progestin and adipoQ receptor family | protein-coding |
| | | | member 6 | |
| chr1 | 156215708 | PRdelta | progestin and adipoQ receptor family | protein-coding |
| | | | member 6 | |
| chr1 | 156215720 | PRdelta | progestin and adipoQ receptor family | protein-coding |
| | | | member 6 | |
| chr1 | 156215726 | PRdelta | progestin and adipoQ receptor family | protein-coding |
| | | | member 6 | |
| chr1 | 156215737 | PRdelta | progestin and adipoQ receptor family | protein-coding |
| | | | member 6 | |
| chr1 | 203259747 | — | long intergenic non-protein coding RNA | ncRNA |
| | | | 1353 | |
| chr1 | 203259757 | — | long intergenic non-protein coding RNA | ncRNA |
| | | | 1353 | |
| chr1 | 203259770 | — | long intergenic non-protein coding RNA | ncRNA |
| | | | 1353 | |
| chr1 | 203259836 | — | long intergenic non-protein coding RNA | ncRNA |
| | | | 1353 | |
| chr1 | 203259850 | — | long intergenic non-protein coding RNA | ncRNA |
| | | | 1353 | |
| chr1 | 203259921 | — | long intergenic non-protein coding RNA | ncRNA |
| | | | 1353 | |
| chr1 | 236311219 | TM7SF1 | G protein-coupled receptor 137B | protein-coding |
| chr1 | 236311342 | TM7SF1 | G protein-coupled receptor 137B | protein-coding |
| chr1 | 237129554 | MT1P2 | metallothionein 1H like 1 | protein-coding |
| chr1 | 237134163 | MT1P2 | metallothionein 1H like 1 | protein-coding |
| chr1 | 237167480 | MT1P2 | metallothionein 1H like 1 | protein-coding |
| chr1 | 237194084 | ARVC2\|ARVD2\|RYR-2\|RyR\|VTSIP | ryanodine receptor 2 | protein-coding |
| chr1 | 237194093 | ARVC2\|ARVD2\|RYR-2\|RyR\|VTSIP | ryanodine receptor 2 | protein-coding |
| chr1 | 237194098 | ARVC2\|ARVD2\|RYR-2\|RyR\|VTSIP | ryanodine receptor 2 | protein-coding |
| chr1 | 237195314 | ARVC2\|ARVD2\|RYR-2\|RyR\|VTSIP | ryanodine receptor 2 | protein-coding |
| chr1 | 237195411 | ARVC2\|ARVD2\|RYR-2\|RyR\|VTSIP | ryanodine receptor 2 | protein-coding |
| chr1 | 237212336 | ARVC2\|ARVD2\|RYR-2\|RyR\|VTSIP | ryanodine receptor 2 | protein-coding |
| chr1 | 237220609 | ARVC2\|ARVD2\|RYR-2\|RyR\|VTSIP | ryanodine receptor 2 | protein-coding |
| chr1 | 237220641 | ARVC2\|ARVD2\|RYR-2\|RyR\|VTSIP | ryanodine receptor 2 | protein-coding |
| chr1 | 237222098 | ARVC2\|ARVD2\|RYR-2\|RyR\|VTSIP | ryanodine receptor 2 | protein-coding |
| chr1 | 237222106 | ARVC2\|ARVD2\|RYR-2\|RyR\|VTSIP | ryanodine receptor 2 | protein-coding |
| chr1 | 237235121 | ARVC2\|ARVD2\|RYR-2\|RyR\|VTSIP | ryanodine receptor 2 | protein-coding |
| chr1 | 237235204 | ARVC2\|ARVD2\|RYR-2\|RyR\|VTSIP | ryanodine receptor 2 | protein-coding |
| chr1 | 237235522 | ARVC2\|ARVD2\|RYR-2\|RyR\|VTSIP | ryanodine receptor 2 | protein-coding |
| chr1 | 237235567 | ARVC2\|ARVD2\|RYR-2\|RyR\|VTSIP | ryanodine receptor 2 | protein-coding |
| chr1 | 237235607 | ARVC2\|ARVD2\|RYR-2\|RyR\|VTSIP | ryanodine receptor 2 | protein-coding |
| chr1 | 237256273 | ARVC2\|ARVD2\|RYR-2\|RyR\|VTSIP | ryanodine receptor 2 | protein-coding |
| chr1 | 237256316 | ARVC2\|ARVD2\|RYR-2\|RyR\|VTSIP | ryanodine receptor 2 | protein-coding |
| chr1 | 237265786 | ARVC2\|ARVD2\|RYR-2\|RyR\|VTSIP | ryanodine receptor 2 | protein-coding |
| chr1 | 237265877 | ARVC2\|ARVD2\|RYR-2\|RyR\|VTSIP | ryanodine receptor 2 | protein-coding |
| chr1 | 237267786 | ARVC2\|ARVD2\|RYR-2\|RyR\|VTSIP | ryanodine receptor 2 | protein-coding |
| chr1 | 237267812 | ARVC2\|ARVD2\|RYR-2\|RyR\|VTSIP | ryanodine receptor 2 | protein-coding |
| chr1 | 237267886 | ARVC2\|ARVD2\|RYR-2\|RyR\|VTSIP | ryanodine receptor 2 | protein-coding |
| chr1 | 237306208 | ARVC2\|ARVD2\|RYR-2\|RyR\|VTSIP | ryanodine receptor 2 | protein-coding |
| chr1 | 237306260 | ARVC2\|ARVD2\|RYR-2\|RyR\|VTSIP | ryanodine receptor 2 | protein-coding |
| chr1 | 237343254 | ARVC2\|ARVD2\|RYR-2\|RyR\|VTSIP | ryanodine receptor 2 | protein-coding |
| chr1 | 237359169 | ARVC2\|ARVD2\|RYR-2\|RyR\|VTSIP | ryanodine receptor 2 | protein-coding |
| chr1 | 237383690 | ARVC2\|ARVD2\|RYR-2\|RyR\|VTSIP | ryanodine receptor 2 | protein-coding |
| chr1 | 237392760 | ARVC2\|ARVD2\|RYR-2\|RyR\|VTSIP | ryanodine receptor 2 | protein-coding |
| chr1 | 237408076 | ARVC2\|ARVD2\|RYR-2\|RyR\|VTSIP | ryanodine receptor 2 | protein-coding |
| chr1 | 237408091 | ARVC2\|ARVD2\|RYR-2\|RyR\|VTSIP | ryanodine receptor 2 | protein-coding |
| chr1 | 237473057 | — | microRNA 4428 | ncRNA |
| chr1 | 237473071 | — | microRNA 4428 | ncRNA |
| chr1 | 237473091 | — | microRNA 4428 | ncRNA |
| chr1 | 237473164 | — | microRNA 4428 | ncRNA |
| chr1 | 237538162 | — | microRNA 4428 | ncRNA |
| chr1 | 237584276 | — | microRNA 4428 | ncRNA |
| chr1 | 237661252 | — | microRNA 4428 | ncRNA |
| chr1 | 237661262 | — | microRNA 4428 | ncRNA |
| chr1 | 237661322 | — | microRNA 4428 | ncRNA |
| chr1 | 237693526 | — | microRNA 4428 | ncRNA |
| chr1 | 237693534 | — | microRNA 4428 | ncRNA |
| chr1 | 237699930 | — | microRNA 4428 | ncRNA |
| chr1 | 237699938 | — | microRNA 4428 | ncRNA |
| chr1 | 237699988 | — | microRNA 4428 | ncRNA |
| chr1 | 237809365 | — | microRNA 4428 | ncRNA |
| chr1 | 237815260 | — | microRNA 4428 | ncRNA |
| chr1 | 241402493 | mir-3123 | microRNA 3123 | ncRNA |
| chr1 | 241402516 | mir-3123 | microRNA 3123 | ncRNA |
| chr2 | 9248023 | AMAP2\|CENTB3\|DDEF2\|PAG3\|PAP\|Pap- | ArfGAP with SH3 domain, ankyrin repeat | protein-coding |
| | | alpha\|SHAG1 | and PH domain 2 | |
| chr2 | 9248368 | AMAP2\|CENTB3\|DDEF2\|PAG3\|PAP\|Pap- | ArfGAP with SH3 domain, ankyrin repeat | protein-coding |
| | | alpha\|SHAG1 | and PH domain 2 | |
| chr2 | 9248435 | AMAP2\|CENTB3\|DDEF2\|PAG3\|PAP\|Pap- | ArfGAP with SH3 domain, ankyrin repeat | protein-coding |
| | | alpha\|SHAG1 | and PH domain 2 | |
| chr2 | 9248469 | AMAP2\|CENTB3\|DDEF2\|PAG3\|PAP\|Pap- | ArfGAP with SH3 domain, ankyrin repeat | protein-coding |
| | | alpha\|SHAG1 | and PH domain 2 | |
| chr2 | 25299679 | KIAA0953 | EFR3 homolog B | protein-coding |
| chr2 | 25299815 | KIAA0953 | EFR3 homolog B | protein-coding |
| chr2 | 25299928 | KIAA0953 | EFR3 homolog B | protein-coding |
| chr2 | 43102420 | 3-HAO\|HAO\|VCRL1\|h3HAO | 3-hydroxyanthramlate 3,4-dioxygenase | protein-coding |
| chr2 | 43107259 | 3-HAO\|HAO\|VCRL1\|h3HAO | 3-hydroxyanthramlate 3,4-dioxygenase | protein-coding |
| chr2 | 43107400 | 3-HAO\|HAO\|VCRL1\|h3HAO | 3-hydroxyanthramlate 3,4-dioxygenase | protein-coding |
| chr2 | 43107414 | 3-HAO\|HAO\|VCRL1\|h3HAO | 3-hydroxyanthramlate 3,4-dioxygenase | protein-coding |
| chr2 | 43107438 | 3-HAO\|HAO\|VCRL1\|h3HAO | 3-hydroxyanthramlate 3,4-dioxygenase | protein-coding |
| chr2 | 43107456 | 3-HAO\|HAO\|VCRL1\|h3HAO | 3-hydroxyanthramlate 3,4-dioxygenase | protein-coding |
| chr2 | 43107465 | 3-HAO\|HAO\|VCRL1\|h3HAO | 3-hydroxyanthramlate 3,4-dioxygenase | protein-coding |
| chr2 | 43107514 | 3-HAO\|HAO\|VCRL1\|h3HAO | 3-hydroxyanthramlate 3,4-dioxygenase | protein-coding |
| chr2 | 43107532 | 3-HAO\|HAO\|VCRL1\|h3HAO | 3-hydroxyanthramlate 3,4-dioxygenase | protein-coding |
| chr2 | 43107650 | 3-HAO\|HAO\|VCRL1\|h3HAO | 3-hydroxyanthramlate 3,4-dioxygenase | protein-coding |
| chr2 | 45235684 | — | SIX homeobox 2 | protein-coding |
| chr2 | 45235702 | — | SIX homeobox 2 | protein-coding |
| chr2 | 45235710 | — | SIX homeobox 2 | protein-coding |
| chr2 | 207972376 | mir-2355 | microRNA 2355 | ncRNA |
| chr2 | 207972408 | mir-2355 | microRNA 2355 | ncRNA |
| chr2 | 220509654 | AE3\|CAE3/BAE3\|SLC2C | solute carrier family 4 member 3 | protein-coding |
| chr2 | 220509693 | AE3\|CAE3/BAE3\|SLC2C | solute carrier family 4 member 3 | protein-coding |
| chr2 | 220509705 | AE3\|CAE3/BAE3\|SLC2C | solute carrier family 4 member 3 | protein-coding |
| chr2 | 220509776 | AE3\|CAE3/BAE3\|SLC2C | solute carrier family 4 member 3 | protein-coding |
| chr2 | 220509830 | AE3\|CAE3/BAE3\|SLC2C | solute carrier family 4 member 3 | protein-coding |
| chr2 | 241233035 | glypican | glypican 1 | protein-coding |
| chr2 | 241233048 | glypican | glypican 1 | protein-coding |
| chr2 | 241233055 | glypican | glypican 1 | protein-coding |
| chr2 | 241233084 | glypican | glypican 1 | protein-coding |
| chr2 | 241233090 | glypican | glypican 1 | protein-coding |
| chr2 | 241233169 | glypican | glypican 1 | protein-coding |
| chr2 | 241233214 | glypican | glypican 1 | protein-coding |
| chr3 | 111431516 | LL5b\|LL5beta | pleckstrin homology like domain family B | protein-coding |
| | | | member 2 | |
| chr3 | 111431559 | LL5b\|LL5beta | pleckstrin homology like domain family B | protein-coding |
| | | | member 2 | |
| chr4 | 3309964 | — | regulator of G protein signaling 12 | protein-coding |
| chr4 | 3310004 | — | regulator of G protein signaling 12 | protein-coding |
| chr4 | 3311787 | — | regulator of G protein signaling 12 | protein-coding |
| chr4 | 3311917 | — | regulator of G protein signaling 12 | protein-coding |
| chr4 | 7228254 | — | sortilin related VPS10 domain containing | protein-coding |
| | | | receptor 2 | |
| chr4 | 7228263 | — | sortilin related VPS10 domain containing | protein-coding |
| | | | receptor 2 | |
| chr4 | 7228304 | — | sortilin related VPS10 domain containing | protein-coding |
| | | | receptor 2 | |
| chr4 | 7228329 | — | sortilin related VPS10 domain containing | protein-coding |
| | | | receptor 2 | |
| chr4 | 7326173 | — | microRNA 4798 | ncRNA |
| chr4 | 7326188 | — | microRNA 4798 | ncRNA |
| chr4 | 7326270 | — | microRNA 4798 | ncRNA |
| chr4 | 7326284 | — | microRNA 4798 | ncRNA |
| chr4 | 155758143 | CT68 | RNA binding motif protein 46 | protein-coding |
| chr4 | 155763679 | CT68 | RNA binding motif protein 46 | protein-coding |
| chr4 | 155763688 | CT68 | RNA binding motif protein 46 | protein-coding |
| chr4 | 155763695 | CT68 | RNA binding motif protein 46 | protein-coding |
| chr4 | 155812513 | CT68 | RNA binding motif protein 46 | protein-coding |
| chr4 | 155912924 | CT68 | RNA binding motif protein 46 | protein-coding |
| chr4 | 155965520 | NPY2-R | neuropeptide Y receptor Y2 | protein-coding |
| chr4 | 155965532 | NPY2-R | neuropeptide Y receptor Y2 | protein-coding |
| chr4 | 155965538 | NPY2-R | neuropeptide Y receptor Y2 | protein-coding |
| chr4 | 155965544 | NPY2-R | neuropeptide Y receptor Y2 | protein-coding |
| chr4 | 155965553 | NPY2-R | neuropeptide Y receptor Y2 | protein-coding |
| chr4 | 155992200 | NPY2-R | neuropeptide Y receptor Y2 | protein-coding |
| chr4 | 156002533 | NPY2-R | neuropeptide Y receptor Y2 | protein-coding |
| chr5 | 1003895 | Naked2 | NKD inhibitor of WNT signaling pathway | protein-coding |
| | | | 2 | |
| chr5 | 1004045 | Naked2 | NKD inhibitor of WNT signaling pathway | protein-coding |
| | | | 2 | |
| chr5 | 1004051 | Naked2 | NKD inhibitor of WNT signaling pathway | protein-coding |
| | | | 2 | |
| chr5 | 1004064 | Naked2 | NKD inhibitor of WNT signaling pathway | protein-coding |
| | | | 2 | |
| chr5 | 1004077 | Naked2 | NKD inhibitor of WNT signaling pathway | protein-coding |
| | | | 2 | |
| chr5 | 1004089 | Naked2 | NKD inhibitor of WNT signaling pathway | protein-coding |
| | | | 2 | |
| chr5 | 1004118 | Naked2 | NKD inhibitor of WNT signaling pathway | protein-coding |
| | | | 2 | |
| chr5 | 1004128 | Naked2 | NKD inhibitor of WNT signaling pathway | protein-coding |
| | | | 2 | |
| chr5 | 1004135 | Naked2 | NKD inhibitor of WNT signaling pathway | protein-coding |
| | | | 2 | |
| chr5 | 1004148 | Naked2 | NKD inhibitor of WNT signaling pathway | protein-coding |
| | | | 2 | |
| chr5 | 1004157 | Naked2 | NKD inhibitor of WNT signaling pathway | protein-coding |
| | | | 2 | |
| chr5 | 1004184 | Naked2 | NKD inhibitor of WNT signaling pathway | protein-coding |
| | | | 2 | |
| chr5 | 1004198 | Naked2 | NKD inhibitor of WNT signaling pathway | protein-coding |
| | | | 2 | |
| chr5 | 1004234 | Naked2 | NKD inhibitor of WNT signaling pathway | protein-coding |
| | | | 2 | |
| chr5 | 1004243 | Naked2 | NKD inhibitor of WNT signaling pathway | protein-coding |
| | | | 2 | |
| chr5 | 1004259 | Naked2 | NKD inhibitor of WNT signaling pathway | protein-coding |
| | | | 2 | |
| chr5 | 1004269 | Naked2 | NKD inhibitor of WNT signaling pathway | orotein-coding |
| | | | 2 | |
| chr5 | 1004288 | Naked2 | NKD inhibitor of WNT signaling pathway | protein-coding |
| | | | 2 | |
| chr5 | 1004304 | Naked2 | NKD inhibitor of WNT signaling pathway | protein-coding |
| | | | 2 | |
| chr5 | 1004351 | Naked2 | NKD inhibitor of WNT signaling pathway | protein-coding |
| | | | 2 | |
| chr5 | 1004357 | Naked2 | NKD inhibitor of WNT signaling pathway | protein-coding |
| | | | 2 | |
| chr5 | 1004365 | Naked2 | NKD inhibitor of WNT signaling pathway | protein-coding |
| | | | 2 | |
| chr5 | 134827336 | — | TIFA inhibitor | protein-coding |
| chr5 | 134827353 | — | TIFA inhibitor | protein-coding |
| chr5 | 134827364 | — | TIFA inhibitor | protein-coding |
| chr5 | 134827378 | — | TIFA inhibitor | protein-coding |
| chr5 | 138441347 | BAP\|MSS\|ULG5 | SIL1 nucleotide exchange factor | protein-coding |
| chr5 | 138441377 | BAP\|MSS\|ULG5 | SIL1 nucleotide exchange factor | protein-coding |
| chr5 | 138441561 | BAP\|MSS\|ULG5 | SIL1 nucleotide exchange factor | protein-coding |
| chr5 | 179401802 | MIRN340\|hsa-mir-340\|mir-340 | microRNA 340 | ncRNA |
| chr5 | 179401833 | MIRN340\|hsa-mir-340\|mir-340 | microRNA 340 | ncRNA |
| chr5 | 179401843 | MIRN340\|hsa-mir-340\|mir-340 | microRNA 340 | ncRNA |
| chr5 | 179401853 | MIRN340\|hsa-mir-340\|mir-340 | microRNA 340 | ncRNA |
| chr5 | 179401872 | MIRN340\|hsa-mir-340\|mir-340 | microRNA 340 | ncRNA |
| chr5 | 179588495 | — | RasGEF domain family member 1C | protein-coding |
| chr5 | 179588504 | — | RasGEF domain family member 1C | protein-coding |
| chr5 | 179588512 | — | RasGEF domain family member 1C | protein-coding |
| chr5 | 179588528 | — | RasGEF domain family member 1C | protein-coding |
| chr5 | 179588550 | — | RasGEF domain family member 1C | protein-coding |
| chr5 | 179588590 | — | RasGEF domain family member 1C | protein-coding |
| chr5 | 179588604 | — | RasGEF domain family member 1C | protein-coding |
| chr5 | 179588617 | — | RasGEF domain family member 1C | protein-coding |
| chr5 | 179588625 | — | RasGEF domain family member 1C | protein-coding |
| chr5 | 179588631 | — | RasGEF domain family member 1C | protein-coding |
| chr5 | 179588672 | — | RasGEF domain family member 1C | protein-coding |
| chr5 | 179588687 | — | RasGEF domain family member 1C | protein-coding |
| chr5 | 179588704 | — | RasGEF domain family member 1C | protein-coding |
| chr5 | 179588719 | — | RasGEF domain family member 1C | protein-coding |
| chr5 | 179597660 | — | RasGEF domain family member 1C | protein-coding |
| chr5 | 179597771 | — | RasGEF domain family member 1C | protein-coding |
| chr5 | 179597812 | — | RasGEF domain family member 1C | protein-coding |
| chr6 | 64151793 | GLULD1\|LGS | lengsin lens protein with glutamine | protein-coding |
| | | | synthetase domain | |
| chr6 | 157549787 | 6A3-5\|BAF250B\|BRIGHT\|CSS1\|DAN15\|ELD/OSA1\| | AT-rich interaction domain 1B | protein-coding |
| | | MRD12\|OSA2\|P250R | | |
| chr6 | 157549797 | 6A3-5\|BAF250B\|BRIGHT\|CSS1\|DAN15\|ELD/OSA1\| | AT-rich interaction domain 1B | protein-coding |
| | | MRD12\|OSA2\|P250R | | |
| chr6 | 157549848 | 6A3-5\|BAF250B\|BRIGHT\|CSS1\|DAN15\|ELD/OSA1\| | AT-rich interaction domain 1B | protein-coding |
| | | MRD12\|OSA2\|P250R | | |
| chr6 | 157550494 | 6A3-5\|BAF250B\|BRIGHT\|CSS1\|DAN15\|ELD/OSA1\| | AT-rich interaction domain 1B | protein-coding |
| | | MRD12\|OSA2\|P250R | | |
| chr6 | 157550509 | 6A3-5\|BAF250B\|BRIGHT\|CSS1\|DAN15\|ELD/OSA1\| | AT-rich interaction domain 1B | protein-coding |
| | | MRD12\|OSA2\|P250R | | |
| chr7 | 1447696 | JRAB\|MICAL-L2 | MICAL like 2 | protein-coding |
| chr7 | 1447734 | JRAB\|MICAL-L2 | MICAL like 2 | protein-coding |
| chr7 | 1447746 | JRAB\|MICAL-L2 | MICAL like 2 | protein-coding |
| chr7 | 1447760 | JRAB\|MICAL-L2 | MICAL like 2 | protein-coding |
| chr7 | 1447774 | JRAB\|MICAL-L2 | MICAL like 2 | protein-coding |
| chr7 | 1447782 | JRAB\|MICAL-L2 | MICAL like 2 | protein-coding |
| chr7 | 2006576 | MAD1\|PIG9\|TP53I9\|TXBP181 | mitotic arrest deficient 1 like 1 | protein-coding |
| chr7 | 2006674 | MAD1\|PIG9\|TP53I9\|TXBP181 | mitotic arrest deficient 1 like 1 | protein-coding |
| chr7 | 2007262 | MAD1\|PIG9\|TP53I9\|TXBP181 | mitotic arrest deficient 1 like 1 | protein-coding |
| chr7 | 2007279 | MAD1\|PIG9\|TP53I9\|TXBP181 | mitotic arrest deficient 1 like 1 | protein-coding |
| chr7 | 2007309 | MAD1\|PIG9\|TP53I9\|TXBP181 | mitotic arrest deficient 1 like 1 | protein-coding |
| chr7 | 2007394 | MAD1\|PIG9\|TP53I9\|TXBP181 | mitotic arrest deficient 1 like 1 | protein-coding |
| chr7 | 2968280 | NNX3\|RMP\|gep | G protein subunit alpha 12 | protein-coding |
| chr7 | 2968288 | NNX3\|RMP\|gep | G protein subunit alpha 12 | protein-coding |
| chr7 | 2968314 | NNX3\|RMP\|gep | G protein subunit alpha 12 | protein-coding |
| chr7 | 2968320 | NNX3\|RMP\|gep | G protein subunit alpha 12 | protein-coding |
| chr7 | 2968352 | NNX3\|RMP\|gep | G protein subunit alpha 12 | protein-coding |
| chr7 | 2968357 | NNX3\|RMP\|gep | G protein subunit alpha 12 | protein-coding |
| chr7 | 2968405 | NNX3\|RMP\|gep | G protein subunit alpha 12 | protein-coding |
| chr7 | 2968411 | NNX3\|RMP\|gep | G protein subunit alpha 12 | protein-coding |
| chr7 | 2968426 | NNX3\|RMP\|gep | G protein subunit alpha 12 | protein-coding |
| chr7 | 2968440 | NNX3\|RMP\|gep | G protein subunit alpha 12 | protein-coding |
| chr7 | 2968454 | NNX3\|RMP\|gep | G protein subunit alpha 12 | protein-coding |
| chr7 | 2968489 | NNX3\|RMP\|gep | G protein subunit alpha 12 | protein-coding |
| chr7 | 2968504 | NNX3\|RMP\|gep | G protein subunit alpha 12 | protein-coding |
| chr7 | 2968528 | NNX3\|RMP\|gep | G protein subunit alpha 12 | protein-coding |
| chr7 | 2968552 | NNX3\|RMP\|gep | G protein subunit alpha 12 | protein-coding |
| chr7 | 2968558 | NNX3\|RMP\|gep | G protein subunit alpha 12 | protein-coding |
| chr7 | 2968565 | NNX3\|RMP\|gep | G protein subunit alpha 12 | protein-coding |
| chr7 | 2968585 | NNX3\|RMP\|gep | G protein subunit alpha 12 | protein-coding |
| chr7 | 2968593 | NNX3\|RMP\|gep | G protein subunit alpha 12 | protein-coding |
| chr7 | 2968625 | NNX3\|RMP\|gep | G protein subunit alpha 12 | protein-coding |
| chr7 | 2968684 | NNX3\|RMP\|gep | G protein subunit alpha 12 | protein-coding |
| chr7 | 2968706 | NNX3\|RMP\|gep | G protein subunit alpha 12 | protein-coding |
| chr7 | 26667584 | — | chromosome 7 open reading frame 71 | ncRNA |
| chr7 | 26667613 | — | chromosome 7 open reading frame 71 | ncRNA |
| chr7 | 26667674 | — | chromosome 7 open reading frame 71 | ncRNA |
| chr7 | 26667709 | — | chromosome 7 open reading frame 71 | ncRNA |
| chr7 | 35505816 | — | uncharacterized LOC401324 | ncRNA |
| chr7 | 35505825 | — | uncharacterized LOC401324 | ncRNA |
| chr7 | 41086116 | — | long intergenic non-protein coding RNA | ncRNA |
| | | | 1449 | |
| chr7 | 41086123 | — | long intergenic non-protein coding RNA | ncRNA |
| | | | 1449 | |
| chr7 | 41086140 | — | long intergenic non-protein coding RNA | ncRNA |
| | | | 1449 | |
| chr7 | 101622017 | CASP\|CDP\|CDP/Cut\|CDP1\|COY1\|CUTL1\|CUX\|Clox\| | cut like homeobox 1 | protein-coding |
| | | Cux/CDP\|GDDI\|GOLIM6\|Nbla10317\|p100\|p110\|p200\|p75 | | |
| chr7 | 101622057 | CASP\|CDP\|CDP/Cut\|CDP1\|COY1\|CUTL1\|CUX\|Clox\| | cut like homeobox 1 | protein-coding |
| | | Cux/CDP\|GDDI\|GOLIM6\|Nbla10317\|p100\|p110\|p200\|p75 | | |
| chr7 | 101622176 | CASP\|CDP\|CDP/Cut\|CDP1\|COY1\|CUTL1\|CUX\|Clox\| | cut like homeobox 1 | protein-coding |
| | | Cux/CDP\|GDDI\|GOLIM6\|Nbla10317\|p100\|p110\|p200\|p75 | | |
| chr7 | 115992730 | — | uncharacterized LOC102724434 | ncRNA |
| chr7 | 115992814 | — | uncharacterized LOC102724434 | ncRNA |
| chr7 | 115998982 | — | uncharacterized LOC102724434 | ncRNA |
| chr7 | 115999026 | — | uncharacterized LOC102724434 | ncRNA |
| chr7 | 115999448 | — | uncharacterized LOC102724434 | ncRNA |
| chr7 | 115999464 | — | uncharacterized LOC102724434 | ncRNA |
| chr7 | 116099337 | CAV | caveolin 2 | protein-coding |
| chr7 | 150668588 | ERG-1\|ERG1\|H- | potassium voltage-gated channel subfamily | protein-coding |
| | | ERG\|HERG\|HERG1\|Kv11.1\|LQT2\|SQT1 | H member 2 | |
| chr7 | 150669120 | ERG-1\|ERG1\|H- | potassium voltage-gated channel subfamily | protein-coding |
| | | ERG\|HERG\|HERG1\|Kv11.1\|LQT2\|SQT1 | H member 2 | |
| chr7 | 150811991 | AGAP-3\|CENTG3\|CRAG\|MRIP-1\|cnt-g3 | ArfGAP with GTPase domain, ankyrin | protein-coding |
| | | | repeat and PH domain 3 | |
| chr7 | 150812075 | AGAP-3\|CENTG3\|CRAG\|MRIP-1\|cnt-g3 | ArfGAP with GTPase domain, ankyrin | protein-coding |
| | | | repeat and PH domain 3 | |
| chr7 | 150812111 | AGAP-3\|CENTG3\|CRAG\|MRIP-1\|cnt-g3 | ArfGAP with GTPase domain, ankyrin | protein-coding |
| | | | repeat and PH domain 3 | |
| chr7 | 150812201 | AGAP-3\|CENTG3\|CRAG\|MRIP-1\|cnt-g3 | ArfGAP with GTPase domain, ankyrin | protein-coding |
| | | | repeat and PH domain 3 | |
| chr7 | 150812215 | AGAP-3\|CENTG3\|CRAG\|MRIP-1\|cnt-g3 | ArfGAP with GTPase domain, ankyrin | protein-coding |
| | | | repeat and PH domain 3 | |
| chr7 | 150812398 | AGAP-3\|CENTG3\|CRAG\|MRIP-1\|cnt-g3 | ArfGAP with GTPase domain, ankyrin | protein-coding |
| | | | repeat and PH domain 3 | |
| chr7 | 150812660 | AGAP-3\|CENTG3\|CRAG\|MRIP-1\|cnt-g3 | ArfGAP with GTPase domain, ankyrin | protein-coding |
| | | | repeat and PH domain 3 | |
| chr7 | 157423275 | MIRN153-2\|mir-153-2 | microRNA 153-2 | ncRNA |
| chr7 | 157623246 | — | uncharacterized LOC100506585 | ncRNA |
| chr8 | 46929775 | ASNSL1 | asparagine synthetase pseudogene 1 | pseudo |
| chr8 | 46935836 | ASNSL1 | asparagine synthetase pseudogene 1 | pseudo |
| chr8 | 46938035 | ASNSL1 | asparagine synthetase pseudogene 1 | pseudo |
| chr8 | 46938044 | ASNSL1 | asparagine synthetase pseudogene 1 | pseudo |
| chr8 | 46938657 | ASNSL1 | asparagine synthetase pseudogene 1 | pseudo |
| chr8 | 46938693 | ASNSL1 | asparagine synthetase pseudogene 1 | pseudo |
| chr8 | 46938702 | ASNSL1 | asparagine synthetase pseudogene 1 | pseudo |
| chr8 | 46940361 | ASNSL1 | asparagine synthetase pseudogene 1 | pseudo |
| chr8 | 46940406 | ASNSL1 | asparagine synthetase pseudogene 1 | pseudo |
| chr8 | 58062363 | — | long intergenic non-protein coding RNA | ncRNA |
| | | | 1606 | |
| chr8 | 58062426 | — | long intergenic non-protein coding RNA | ncRNA |
| | | | 1606 | |
| chr8 | 58062463 | — | long intergenic non-protein coding RNA | ncRNA |
| | | | 1606 | |
| chr8 | 58096948 | — | long intergenic non-protein coding RNA | ncRNA |
| | | | 1606 | |
| chr8 | 58115010 | — | long intergenic non-protein coding RNA | ncRNA |
| | | | 1606 | |
| chr8 | 58115060 | — | long intergenic non-protein coding RNA | ncRNA |
| | | | 1606 | |
| chr8 | 58115154 | — | long intergenic non-protein coding RNA | ncRNA |
| | | | 1606 | |
| chr8 | 58115225 | — | long intergenic non-protein coding RNA | ncRNA |
| | | | 1606 | |
| chr8 | 58115343 | — | long intergenic non-protein coding RNA | ncRNA |
| | | | 1606 | |
| chr8 | 58115362 | — | long intergenic non-protein coding RNA | ncRNA |
| | | | 1606 | |
| chr8 | 58122819 | — | long intergenic non-protein coding RNA | ncRNA |
| | | | 1606 | |
| chr8 | 58122836 | — | long intergenic non-protein coding RNA | ncRNA |
| | | | 1606 | |
| chr8 | 58122848 | — | long intergenic non-protein coding RNA | ncRNA |
| | | | 1606 | |
| chr8 | 58122861 | — | long intergenic non-protein coding RNA | ncRNA |
| | | | 1606 | |
| chr8 | 58123008 | — | long intergenic non-protein coding RNA | ncRNA |
| | | | 1606 | |
| chr8 | 58126620 | — | long intergenic non-protein coding RNA | ncRNA |
| | | | 1606 | |
| chr8 | 58126666 | — | long intergenic non-protein coding RNA | ncRNA |
| | | | 1606 | |
| chr8 | 58126874 | — | long intergenic non-protein coding RNA | ncRNA |
| | | | 1606 | |
| chr8 | 58127096 | — | long intergenic non-protein coding RNA | ncRNA |
| | | | 1606 | |
| chr8 | 58127515 | — | long intergenic non-protein coding RNA | ncRNA |
| | | | 1606 | |
| chr8 | 58127556 | — | long intergenic non-protein coding RNA | ncRNA |
| | | | 1606 | |
| chr8 | 58127613 | — | long intergenic non-protein coding RNA | ncRNA |
| | | | 1606 | |
| chr8 | 58130004 | — | long intergenic non-protein coding RNA | ncRNA |
| | | | 1606 | |
| chr8 | 58130021 | — | long intergenic non-protein coding RNA | ncRNA |
| | | | 1606 | |
| chr8 | 58130039 | — | long intergenic non-protein coding RNA | ncRNA |
| | | | 1606 | |
| chr8 | 58131654 | — | long intergenic non-protein coding RNA | ncRNA |
| | | | 1606 | |
| chr8 | 58131665 | — | long intergenic non-protein coding RNA | ncRNA |
| | | | 1606 | |
| chr8 | 58131710 | — | long intergenic non-protein coding RNA | ncRNA |
| | | | 1606 | |
| chr8 | 58131715 | — | long intergenic non-protein coding RNA | ncRNA |
| | | | 1606 | |
| chr8 | 58131843 | — | long intergenic non-protein coding RNA | ncRNA |
| | | | 1606 | |
| chr8 | 58131870 | — | long intergenic non-protein coding RNA | ncRNA |
| | | | 1606 | |
| chr8 | 58133184 | — | long intergenic non-protein coding RNA | ncRNA |
| | | | 1606 | |
| chr8 | 58133215 | — | long intergenic non-protein coding RNA | ncRNA |
| | | | 1606 | |
| chr8 | 58133315 | — | long intergenic non-protein coding RNA | ncRNA |
| | | | 1606 | |
| chr8 | 58133743 | — | long intergenic non-protein coding RNA | ncRNA |
| | | | 1606 | |
| chr8 | 58171196 | — | uncharacterized LOC286177 | ncRNA |
| chr8 | 58171236 | — | uncharacterized LOC286177 | ncRNA |
| chr8 | 58192020 | C8orf71 | long intergenic non-protein coding RNA | ncRNA |
| | | | 588 | |
| chr8 | 61777862 | — | hCG1816373-like | ncRNA |
| chr8 | 61821531 | — | hCG1816373-like | ncRNA |
| chr8 | 61821546 | — | hCG1816373-like | ncRNA |
| chr8 | 61821578 | — | hCG1816373-like | ncRNA |
| chr8 | 73471538 | KV2.2 | potassium voltage-gated channel subfamily | protein-coding |
| | | | B member 2 | |
| chr8 | 73480053 | KV2.2 | potassium voltage-gated channel subfamily | protein-coding |
| | | | B member 2 | |
| chr8 | 73551842 | — | uncharacterized LOC101926908 | ncRNA |
| chr8 | 73849013 | PIN2\|TRBF1\|TRF\|TRF1\|hTRF1-AS\|t-TRF1 | telomeric repeat binding factor 1 | protein-coding |
| chr8 | 73849032 | PIN2\|TRBF1\|TRF\|TRF1\|hTRF1-AS\|t-TRF1 | telomeric repeat binding factor 1 | protein-coding |
| chr8 | 73849038 | PIN2\|TRBF1\|TRF\|TRF1\|hTRF1-AS\|t-TRF1 | telomeric repeat binding factor 1 | protein-coding |
| chr8 | 73849051 | PIN2\|TRBF1\|TRF\|TRF1\|hTRF1-AS\|t-TRF1 | telomeric repeat binding factor 1 | protein-coding |
| chr8 | 73849060 | PIN2\|TRBF1\|TRF\|TRF1\|hTRF1-AS\|t-TRF1 | telomeric repeat binding factor 1 | protein-coding |
| chr8 | 104117894 | — | long intergenic non-protein coding RNA | ncRNA |
| | | | 1181 | |
| chr8 | 140929837 | — | paternally expressed 13 | ncRNA |
| chr8 | 140930939 | — | paternally expressed 13 | ncRNA |
| chr8 | 140931316 | — | paternally expressed 13 | ncRNA |
| chr8 | 142367903 | — | G protein-coupled receptor 20 | protein-coding |
| chr8 | 142367951 | — | G protein-coupled receptor 20 | protein-coding |
| chr8 | 142367989 | — | G protein-coupled receptor 20 | protein-coding |
| chr9 | 117049844 | AGP-A\|AGP1\|HEL-S-153w\|ORM | orosomucoid 1 | protein-coding |
| chr9 | 117049850 | AGP-A\|AGP1\|HEL-S-153w\|ORM | orosomucoid 1 | protein-coding |
| chr9 | 117049862 | AGP-A\|AGP1\|HEL-S-153w\|ORM | orosomucoid 1 | protein-coding |
| chr9 | 117049885 | AGP-A\|AGP1\|HEL-S-153w\|ORM | orosomucoid 1 | protein-coding |
| chr9 | 127105722 | — | uncharacterized LOC100129034 | ncRNA |
| chr9 | 127105763 | — | uncharacterized LOC100129034 | ncRNA |
| chr9 | 129895923 | ARP2\|HARP | angiopoietin like 2 | protein-coding |
| chr9 | 136007375 | RGDS\|RGF\|RalGEF | ral guanine nucleotide dissociation | protein-coding |
| | | | stimulator | |
| chr9 | 136007484 | RGDS\|RGF\|RalGEF | ral guanine nucleotide dissociation | protein-coding |
| | | | stimulator | |
| chr9 | 136007594 | RGDS\|RGF\|RalGEF | ral guanine nucleotide dissociation | protein-coding |
| | | | stimulator | |
| chr9 | 136007599 | RGDS\|RGF\|RalGEF | ral guanine nucleotide dissociation | protein-coding |
| | | | stimulator | |
| chr9 | 136007615 | RGDS\|RGF\|RalGEF | ral guanine nucleotide dissociation | protein-coding |
| | | | stimulator | |
| chr9 | 136007712 | RGDS\|RGF\|RalGEF | ral guanine nucleotide dissociation | protein-coding |
| | | | stimulator | |
| chr9 | 136324027 | C9orf7\|D9S2135\|FLOWER | calcium channel flower domain containing | protein-coding |
| | | | 1 | |
| chr9 | 136324037 | C9orf7\|D9S2135\|FLOWER | calcium channel flower domain containing | protein-coding |
| | | | 1 | |
| chr9 | 136324064 | C9orf7\|D9S2135\|FLOWER | calcium channel flower domain containing | protein-coding |
| | | | 1 | |
| chr9 | 138688868 | EIEE14\|ENFL5\|KCa4.1\|SLACK\|Slo2.2\|bA100C15.2 | potassium sodium-activated channel | protein-coding |
| | | | subfamily T member 1 | |
| chr9 | 138688876 | EIEE14\|ENFL5\|KCa4.1\|SLACK\|Slo2.2\|bA100C15.2 | potassium sodium-activated channel | protein-coding |
| | | | subfamily T member 1 | |
| chr9 | 138688902 | EIEE14\|ENFL5\|KCa4.1\|SLACK\|Slo2.2\|bA100C15.2 | potassium sodium-activated channel | protein-coding |
| | | | subfamily T member 1 | |
| chr9 | 138688936 | EIEE14\|ENFL5\|KCa4.1\|SLACK\|Slo2.2\|bA100C15.2 | potassium sodium-activated channel | protein-coding |
| | | | subfamily T member 1 | |
| chr9 | 138689094 | EIEE14\|ENFL5\|KCa4.1\|SLACK\|Slo2.2\|bA100C15.2 | potassium sodium-activated channel | protein-coding |
| | | | subfamily T member 1 | |
| chr9 | 138689107 | EIEE14\|ENFL5\|KCa4.1\|SLACK\|Slo2.2\|bA100C15.2 | potassium sodium-activated channel | protein-coding |
| | | | subfamily T member 1 | |
| chr9 | 138689483 | EIEE14\|ENFL5\|KCa4.1\|SLACK\|Slo2.2\|bA100C15.2 | potassium sodium-activated channel | protein-coding |
| | | | subfamily T member 1 | |
| chr9 | 138719312 | — | calmodulin regulated spectrin associated | protein-coding |
| | | | protein 1 | |
| chr9 | 138719386 | — | calmodulin regulated spectrin associated | protein-coding |
| | | | protein 1 | |
| chr9 | 139419669 | — | microRNA 4673 | ncRNA |
| chr9 | 139419769 | — | microRNA 4673 | ncRNA |
| chr9 | 139419814 | — | microRNA 4673 | ncRNA |
| chr9 | 139419865 | — | microRNA 4673 | ncRNA |
| chr9 | 139419924 | — | microRNA 4673 | ncRNA |
| chr9 | 139419933 | — | microRNA 4673 | ncRNA |
| chr9 | 139419956 | — | microRNA 4673 | ncRNA |
| chr9 | 139420550 | — | microRNA 4673 | ncRNA |
| chr9 | 139420561 | — | microRNA 4673 | ncRNA |
| chr9 | 139420578 | — | microRNA 4673 | ncRNA |
| chr9 | 139420602 | — | microRNA 4673 | ncRNA |
| chr9 | 139420641 | — | microRNA 4673 | ncRNA |
| chr9 | 139420862 | — | microRNA 4673 | ncRNA |
| chr9 | 139420914 | — | microRNA 4673 | ncRNA |
| chr9 | 139421144 | — | microRNA 4673 | ncRNA |
| chr9 | 139421220 | — | microRNA 4673 | ncRNA |
| chr9 | 139421241 | — | microRNA 4673 | ncRNA |
| chr9 | 139421257 | — | microRNA 4673 | ncRNA |
| chr9 | 139421267 | — | microRNA 4673 | ncRNA |
| chr9 | 139421321 | — | microRNA 4673 | ncRNA |
| chr9 | 139421342 | — | microRNA 4673 | ncRNA |
| chr9 | 139421362 | — | microRNA 4673 | ncRNA |
| chr9 | 139422178 | — | microRNA 4673 | ncRNA |
| chr9 | 139422203 | — | microRNA 4673 | ncRNA |
| chr9 | 139422218 | — | microRNA 4673 | ncRNA |
| chr9 | 139422266 | — | microRNA 4673 | ncRNA |
| chr9 | 139422290 | — | microRNA 4673 | ncRNA |
| chr9 | 139422314 | — | microRNA 4673 | ncRNA |
| chr10 | 23976532 | EIL4\|SKT | KIAA1217 | protein-coding |
| chr10 | 23976581 | EIL4\|SKT | KIAA1217 | protein-coding |
| chr10 | 72489844 | C10orf27\|SPATIAL | thymus, brain and testes associated | protein-coding |
| chr10 | 72489870 | C10orf27\|SPATIAL | thymus, brain and testes associated | protein-coding |
| chr10 | 72489889 | C10orf27\|SPATIAL | thymus, brain and testes associated | protein-coding |
| chr10 | 72489949 | C10orf27\|SPATIAL | thymus, brain and testes associated | protein-coding |
| chr10 | 72489967 | C10orf27\|SPATIAL | thymus, brain and testes associated | protein-coding |
| chr10 | 72489983 | C10orf27\|SPATIAL | thymus, brain and testes associated | protein-coding |
| chr10 | 119590052 | C10orf5 | cancer susceptibility 2 | ncRNA |
| chr10 | 134562610 | GTX\|NKX6.2\|NKX6B\|SPAX8 | NK6 homeobox 2 | protein-coding |
| chr10 | 134563052 | GTX\|NKX6.2\|NKX6B\|SPAX8 | NK6 homeobox 2 | protein-coding |
| chr10 | 134563060 | GTX\|NKX6.2\|NKX6B\|SPAX8 | NK6 homeobox 2 | protein-coding |
| chr10 | 134563098 | GTX\|NKX6.2\|NKX6B\|SPAX8 | NK6 homeobox 2 | protein-coding |
| chr10 | 134563109 | GTX\|NKX6.2\|NKX6B\|SPAX8 | NK6 homeobox 2 | protein-coding |
| chr10 | 134563144 | GTX\|NKX6.2\|NKX6B\|SPAX8 | NK6 homeobox 2 | protein-coding |
| chr10 | 134563149 | GTX\|NKX6.2\|NKX6B\|SPAX8 | NK6 homeobox 2 | protein-coding |
| chr10 | 134563186 | GTX\|NKX6.2\|NKX6B\|SPAX8 | NK6 homeobox 2 | protein-coding |
| chr10 | 134563199 | GTX\|NKX6.2\|NKX6B\|SPAX8 | NK6 homeobox 2 | protein-coding |
| chr11 | 2495501 | ATFB1\|ATFB3\|JLNS1\|KCNA8\|KCNA9\|KVLQT1\| | potassium voltage-gated channel subfamily | protein-coding |
| | | Kv1.9\|Kv7.1\|LQT\|LQT1\|RWS\|SQT2\|WRS | Q member 1 | |
| chr11 | 2495543 | ATFB1\|ATFB3\|JLNS1\|KCNA8\|KCNA9\|KVLQT1\| | potassium voltage-gated channel subfamily | protein-coding |
| | | Kv1.9\|Kv7.1\|LQT\|LQT1\|RWS\|SQT2\|WRS | Q member 1 | |
| chr11 | 2495585 | ATFB1\|ATFB3\|JLNS1\|KCNA8\|KCNA9\|KVLQT1\| | potassium voltage-gated channel subfamily | protein-coding |
| | | Kv1.9\|Kv7.1\|LQT\|LQT1\|RWS\|SQT2\|WRS | Q member 1 | |
| chr11 | 2495609 | ATFB1\|ATFB3\|JLNS1\|KCNA8\|KCNA9\|KVLQT1\| | potassium voltage-gated channel subfamily | protein-coding |
| | | Kv1.9\|Kv7.1\|LQT\|LQT1\|RWS\|SQT2\|WRS | Q member 1 | |
| chr11 | 68967954 | — | uncharacterized LOC338694 | ncRNA |
| chr11 | 68967994 | — | uncharacterized LOC338694 | ncRNA |
| chr11 | 68968016 | — | uncharacterized LOC338694 | ncRNA |
| chr11 | 68968032 | — | uncharacterized LOC338694 | ncRNA |
| chr11 | 68968042 | — | uncharacterized LOC338694 | ncRNA |
| chr11 | 68968141 | — | uncharacterized LOC338694 | ncRNA |
| chr11 | 68968151 | — | uncharacterized LOC338694 | ncRNA |
| chr11 | 68968156 | — | uncharacterized LOC338694 | ncRNA |
| chr11 | 68968182 | — | uncharacterized LOC338694 | ncRNA |
| chr11 | 68968214 | — | uncharacterized LOC338694 | ncRNA |
| chr11 | 68968240 | — | uncharacterized LOC338694 | ncRNA |
| chr11 | 68968267 | — | uncharacterized LOC338694 | ncRNA |
| chr11 | 70851529 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70851574 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70851603 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70851619 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70851638 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70851713 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70851722 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70851730 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70851827 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70851836 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70851859 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70851868 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70851883 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70851889 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70851914 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70862301 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70862379 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70862397 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 70862405 | AUTS17\|CORTBP1\|CTTNBP1\|ProSAP1\| | SH3 and multiple ankyrin repeat domains 2 | protein-coding |
| | | SHANK\|SPANK-3 | | |
| chr11 | 111265961 | BOB1\|OBF-1\|OBF1\|OCAB | POU class 2 homeobox associating factor 1 | protein-coding |
| chr11 | 111266000 | BOB1\|OBF-1\|OBF1\|OCAB | POU class 2 homeobox associating factor 1 | protein-coding |
| chr11 | 111266014 | BOB1\|OBF-1\|OBF1\|OCAB | POU class 2 homeobox associating factor 1 | protein-coding |
| chr11 | 111266031 | BOB1\|OBF-1\|OBF1\|OCAB | POU class 2 homeobox associating factor 1 | protein-coding |
| chr11 | 111266119 | BOB1\|OBF-1\|OBF1\|OCAB | POU class 2 homeobox associating factor 1 | protein-coding |
| chr11 | 115829517 | — | long intergenic non-protein coding RNA | ncRNA |
| | | | 900 | |
| chr11 | 115829550 | — | long intergenic non-protein coding RNA | ncRNA |
| | | | 900 | |
| chr11 | 115830020 | — | long intergenic non-protein coding RNA | ncRNA |
| | | | 900 | |
| chr11 | 115830200 | — | long intergenic non-protein coding RNA | ncRNA |
| | | | 900 | |
| chr11 | 133902554 | — | long intergenic non-protein coding RNA | ncRNA |
| | | | 2731 | |
| chr11 | 133902596 | — | long intergenic non-protein coding RNA | ncRNA |
| | | | 2731 | |
| chr11 | 133902615 | — | long intergenic non-protein coding RNA | ncRNA |
| | | | 2731 | |
| chr11 | 133902626 | — | long intergenic non-protein coding RNA | ncRNA |
| | | | 2731 | |
| chr11 | 133902656 | — | long intergenic non-protein coding RNA | ncRNA |
| | | | 2731 | |
| chr11 | 133902669 | — | long intergenic non-protein coding RNA | ncRNA |
| | | | 2731 | |
| chr11 | 133902689 | — | long intergenic non-protein coding RNA | ncRNA |
| | | | 2731 | |
| chr11 | 133902724 | — | long intergenic non-protein coding RNA | ncRNA |
| | | | 2731 | |
| chr11 | 133902737 | — | long intergenic non-protein coding RNA | ncRNA |
| | | | 2731 | |
| chr11 | 133902744 | — | long intergenic non-protein coding RNA | ncRNA |
| | | | 2731 | |
| chr11 | 133902766 | — | long intergenic non-protein coding RNA | ncRNA |
| | | | 2731 | |
| chr12 | 1957456 | — | leucine rich repeats and transmembrane | protein-coding |
| | | | domains 2 | |
| chr12 | 1957466 | — | leucine rich repeats and transmembrane | protein-coding |
| | | | domains 2 | |
| chr12 | 1957486 | — | leucine rich repeats and transmembrane | protein-coding |
| | | | domains 2 | |
| chr12 | 1957501 | — | leucine rich repeats and transmembrane | protein-coding |
| | | | domains 2 | |
| chr12 | 1957566 | — | leucine rich repeats and transmembrane | protein-coding |
| | | | domains 2 | |
| chr12 | 34488345 | ALG1OA\|DIE2\|KCR1 | ALG10 alpha-1,2-glucosyltransferase | protein-coding |
| chr12 | 34488352 | ALG1OA\|DIE2\|KCR1 | ALG10 alpha-1,2-glucosyltransferase | protein-coding |
| chr12 | 66111480 | — | uncharacterized LOC100507065 | ncRNA |
| chr12 | 66111548 | — | uncharacterized LOC100507065 | ncRNA |
| chr12 | 66114657 | — | uncharacterized LOC100507065 | ncRNA |
| chr12 | 66114687 | — | uncharacterized LOC100507065 | ncRNA |
| chr12 | 66114695 | — | uncharacterized LOC100507065 | ncRNA |
| chr12 | 66262212 | — | HMGA2 antisense RNA 1 | ncRNA |
| chr12 | 66262228 | — | HMGA2 antisense RNA 1 | ncRNA |
| chr12 | 115109512 | TBX3-ISO\|UMS\|XHL | T-box transcription factor 3 | protein-coding |
| chr12 | 115109529 | TBX3-ISO\|UMS\|XHL | T-box transcription factor 3 | protein-coding |
| chr12 | 115109718 | TBX3-ISO\|UMS\|XHL | T-box transcription factor 3 | protein-coding |
| chr12 | 115109960 | TBX3-ISO\|UMS\|XHL | T-box transcription factor 3 | protein-coding |
| chr12 | 115112024 | TBX3-ISO\|UMS\|XHL | T-box transcription factor 3 | protein-coding |
| chr12 | 115112037 | TBX3-ISO\|UMS\|XHL | T-box transcription factor 3 | protein-coding |
| chr12 | 124947867 | CTG26\|N-CoR2\|SMAP270\|SMRT\|SMRTE\|SMRTE- | nuclear receptor corepressor 2 | protein-coding |
| | | tau\|TNRC14\|TRAC\|TRAC-1\|TRAC1 | | |
| chr12 | 124948139 | CTG26\|N-CoR2\|SMAP270\|SMRT\|SMRTE\|SMRTE- | nuclear receptor corepressor 2 | protein-coding |
| | | tau\|TNRC14\|TRAC\|TRAC-1\|TRAC1 | | |
| chr13 | 114523664 | — | GAS6 antisense RNA 1 | ncRNA |
| chr13 | 114523669 | — | GAS6 antisense RNA 1 | ncRNA |
| chr13 | 114523717 | — | GAS6 antisense RNA 1 | ncRNA |
| chr13 | 114523760 | — | GAS6 antisense RNA 1 | ncRNA |
| chr13 | 114523787 | — | GAS6 antisense RNA 1 | ncRNA |
| chr13 | 114523796 | — | GAS6 antisense RNA 1 | ncRNA |
| chr13 | 114523819 | — | GAS6 antisense RNA 1 | ncRNA |
| chr13 | 114523826 | — | GAS6 antisense RNA 1 | ncRNA |
| chr13 | 114523875 | — | GAS6 antisense RNA 1 | ncRNA |
| chr13 | 114523883 | — | GAS6 antisense RNA 1 | ncRNA |
| chr13 | 114523896 | — | GAS6 antisense RNA 1 | ncRNA |
| chr13 | 114523904 | — | GAS6 antisense RNA 1 | ncRNA |
| chr13 | 114523923 | — | GAS6 antisense RNA 1 | ncRNA |
| chr13 | 114523948 | — | GAS6 antisense RNA 1 | ncRNA |
| chr13 | 114523958 | — | GAS6 antisense RNA 1 | ncRNA |
| chr13 | 114523970 | — | GAS6 antisense RNA 1 | ncRNA |
| chr13 | 114523982 | — | GAS6 antisense RNA 1 | ncRNA |
| chr13 | 114523996 | — | GAS6 antisense RNA 1 | ncRNA |
| chr13 | 114524029 | — | GAS6 antisense RNA 1 | ncRNA |
| chr13 | 114524093 | — | GAS6 antisense RNA 1 | ncRNA |
| chr13 | 114524130 | — | GAS6 antisense RNA 1 | ncRNA |
| chr13 | 114524137 | — | GAS6 antisense RNA 1 | ncRNA |
| chr13 | 114524149 | — | GAS6 antisense RNA 1 | ncRNA |
| chr13 | 114524157 | — | GAS6 antisense RNA 1 | ncRNA |
| chr13 | 114524420 | — | GAS6 antisense RNA 1 | ncRNA |
| chr13 | 114524429 | — | GAS6 antisense RNA 1 | ncRNA |
| chr13 | 114524439 | — | GAS6 antisense RNA 1 | ncRNA |
| chr13 | 114524473 | — | GAS6 antisense RNA 1 | ncRNA |
| chr13 | 114524509 | — | GAS6 antisense RNA 1 | ncRNA |
| chr13 | 114524517 | — | GAS6 antisense RNA 1 | ncRNA |
| chr13 | 114524583 | — | GAS6 antisense RNA 1 | ncRNA |
| chr14 | 20684518 | — | olfactory receptor family 11 subfamily H | protein-coding |
| | | | member 6 | |
| chr14 | 20684528 | — | olfactory receptor family 11 subfamily H | protein-coding |
| | | | member 6 | |
| chr14 | 76957115 | DFNB35\|ERR2\|ERRb\|ESRL2\|NR3B2 | estrogen related receptor beta | protein-coding |
| chr14 | 76957138 | DFNB35\|ERR2\|ERRb\|ESRL2\|NR3B2 | estrogen related receptor beta | protein-coding |
| chr14 | 76957152 | DFNB35\|ERR2\|ERRb\|ESRL2\|NR3B2 | estrogen related receptor beta | protein-coding |
| chr14 | 76957199 | DFNB35\|ERR2\|ERRb\|ESRL2\|NR3B2 | estrogen related receptor beta | protein-coding |
| chr14 | 93423461 | CGA | chromogranin A | protein-coding |
| chr14 | 93423503 | CGA | chromogranin A | protein-coding |
| chr14 | 93423558 | CGA | chromogranin A | protein-coding |
| chr14 | 93425210 | CGA | chromogranin A | protein-coding |
| chr14 | 93425284 | CGA | chromogranin A | protein-coding |
| chr14 | 93425331 | CGA | chromogranin A | protein-coding |
| chr14 | 93425520 | CGA | chromogranin A | protein-coding |
| chr14 | 93425529 | CGA | chromogranin A | protein-coding |
| chr14 | 93425655 | CGA | chromogranin A | protein-coding |
| chr14 | 93425687 | CGA | chromogranin A | protein-coding |
| chr14 | 93425735 | CGA | chromogranin A | protein-coding |
| chr14 | 93425741 | CGA | chromogranin A | protein-coding |
| chr14 | 93426221 | CGA | chromogranin A | protein-coding |
| chr14 | 93426229 | CGA | chromogranin A | protein-coding |
| chr14 | 93426247 | CGA | chromogranin A | protein-coding |
| chr14 | 93426257 | CGA | chromogranin A | protein-coding |
| chr14 | 93426336 | CGA | chromogranin A | protein-coding |
| chr14 | 93426381 | CGA | chromogranin A | protein-coding |
| chr14 | 93426456 | CGA | chromogranin A | protein-coding |
| chr14 | 93426481 | CGA | chromogranin A | protein-coding |
| chr14 | 93426499 | CGA | chromogranin A | protein-coding |
| chr14 | 93426875 | CGA | chromogranin A | protein-coding |
| chr14 | 93426916 | CGA | chromogranin A | protein-coding |
| chr14 | 101158277 | C14orf70 | long intergenic non-protein coding RNA | ncRNA |
| | | | 523 | |
| chr14 | 101158294 | C14orf70 | long intergenic non-protein coding RNA | ncRNA |
| | | | 523 | |
| chr14 | 101159404 | DLK\|DLK-1\|Delta1\|FA1\|PREF1\|Pref-1\|ZOG\|pG2 | delta like non-canonical Notch ligand 1 | protein-coding |
| chr14 | 101159430 | DLK\|DLK-1\|Delta1\|FA1\|PREF1\|Pref-1\|ZOG\|pG2 | delta like non-canonical Notch ligand 1 | protein-coding |
| chr14 | 101159551 | DLK\|DLK-1\|Delta1\|FA1\|PREF1\|Pref-1\|ZOG\|pG2 | delta like non-canonical Notch ligand 1 | protein-coding |
| chr14 | 101250153 | — | microRNA 2392 | ncRNA |
| chr14 | 101250165 | — | microRNA 2392 | ncRNA |
| chr14 | 101250240 | — | microRNA 2392 | ncRNA |
| chr14 | 101250254 | — | microRNA 2392 | ncRNA |
| chr14 | 101250279 | — | microRNA 2392 | ncRNA |
| chr14 | 101640895 | — | long intergenic non-protein coding RNA | ncRNA |
| | | | 2285 | |
| chr14 | 101640910 | — | long intergenic non-protein coding RNA | ncRNA |
| | | | 2285 | |
| chr14 | 101640942 | — | long intergenic non-protein coding RNA | ncRNA |
| | | | 2285 | |
| chr14 | 101640959 | — | long intergenic non-protein coding RNA | ncRNA |
| | | | 2285 | |
| chr14 | 105347685 | — | microRNA 12121 | ncRNA |
| chr14 | 105347700 | — | microRNA 12121 | ncRNA |
| chr14 | 105347712 | — | microRNA 12121 | ncRNA |
| chr14 | 105347802 | — | microRNA 12121 | ncRNA |
| chr14 | 105347810 | — | microRNA 12121 | ncRNA |
| chr14 | 105347834 | — | microRNA 12121 | ncRNA |
| chr14 | 105347851 | — | microRNA 12121 | ncRNA |
| chr14 | 105347961 | — | microRNA 12121 | ncRNA |
| chr15 | 22559959 | — | arginine-glutamic acid dipeptide repeats | pseudo |
| | | | pseudogene 3 | |
| chr15 | 22559966 | — | arginine-glutamic acid dipeptide repeats | pseudo |
| | | | pseudogene 3 | |
| chr15 | 22567152 | — | arginine-glutamic acid dipeptide repeats | pseudo |
| | | | pseudogene 3 | |
| chr15 | 22567172 | — | arginine-glutamic acid dipeptide repeats | pseudo |
| | | | pseudogene 3 | |
| chr15 | 22571751 | — | arginine-glutamic acid dipeptide repeats | pseudo |
| | | | pseudogene 3 | |
| chr15 | 22571783 | — | arginine-glutamic acid dipeptide repeats | pseudo |
| | | | pseudogene 3 | |
| chr15 | 22581845 | — | arginine-glutamic acid dipeptide repeats | pseudo |
| | | | pseudogene 3 | |
| chr15 | 22581981 | — | arginine-glutamic acid dipeptide repeats | pseudo |
| | | | pseudogene 3 | |
| chr15 | 74521143 | CT61\|HP11097 | coiled-coil domain containing 33 | protein-coding |
| chr15 | 74521156 | CT61\|HP11097 | coiled-coil domain containing 33 | protein-coding |
| chr15 | 74521297 | CT61\|HP11097 | coiled-coil domain containing 33 | protein-coding |
| chr15 | 74521344 | CT61\|HP11097 | coiled-coil domain containing 33 | protein-coding |
| chr15 | 74521357 | CT61\|HP11097 | coiled-coil domain containing 33 | protein-coding |
| chr15 | 74521364 | CT61\|HP11097 | coiled-coil domain containing 33 | protein-coding |
| chr15 | 94613752 | — | long intergenic non-protein coding RNA | ncRNA |
| | | | 1581 | |
| chr15 | 94613766 | — | long intergenic non-protein coding RNA | ncRNA |
| | | | 1581 | |
| chr15 | 94613787 | — | long intergenic non-protein coding RNA | ncRNA |
| | | | 1581 | |
| chr15 | 94613823 | — | long intergenic non-protein coding RNA | ncRNA |
| | | | 1581 | |
| chr15 | 94613901 | — | long intergenic non-protein coding RNA | ncRNA |
| | | | 1581 | |
| chr17 | 915606 | mir-3183 | microRNA 3183 | ncRNA |
| chr17 | 915643 | mir-3183 | microRNA 3183 | ncRNA |
| chr17 | 915806 | mir-3183 | microRNA 3183 | ncRNA |
| chr17 | 915890 | mir-3183 | microRNA 3183 | ncRNA |
| chr17 | 915912 | mir-3183 | microRNA 3183 | ncRNA |
| chr17 | 7010397 | ASGP-R2\|ASGPR2\|CLEC4H2\|HBXBP\|HL-2 | asialoglycoprotein receptor 2 | protein-coding |
| chr17 | 7010445 | ASGP-R2\|ASGPR2\|CLEC4H2\|HBXBP\|HL-2 | asialoglycoprotein receptor 2 | protein-coding |
| chr17 | 7010504 | ASGP-R2\|ASGPR2\|CLEC4H2\|HBXBP\|HL-2 | asialoglycoprotein receptor 2 | protein-coding |
| chr17 | 7010533 | ASGP-R2\|ASGPR2\|CLEC4H2\|HBXBP\|HL-2 | asialoglycoprotein receptor 2 | protein-coding |
| chr17 | 7010548 | ASGP-R2\|ASGPR2\|CLEC4H2\|HBXBP\|HL-2 | asialoglycoprotein receptor 2 | protein-coding |
| chr17 | 7010630 | ASGP-R2\|ASGPR2\|CLEC4H2\|HBXBP\|HL-2 | asialoglycoprotein receptor 2 | protein-coding |
| chr17 | 7010663 | ASGP-R2\|ASGPR2\|CLEC4H2\|HBXBP\|HL-2 | asialoglycoprotein receptor 2 | protein-coding |
| chr17 | 10728383 | — | phosphoinositide interacting regulator of | protein-coding |
| | | | transient receptor potential channels | |
| chr17 | 10740512 | — | phosphoinositide interacting regulator of | protein-coding |
| | | | transient receptor potential channels | |
| chr17 | 10740558 | — | phosphoinositide interacting regulator of | protein-coding |
| | | | transient receptor potential channels | |
| chr17 | 10740569 | — | phosphoinositide interacting regulator of | protein-coding |
| | | | transient receptor potential channels | |
| chr17 | 10740581 | — | phosphoinositide interacting regulator of | protein-coding |
| | | | transient receptor potential channels | |
| chr17 | 10740595 | — | phosphoinositide interacting regulator of | protein-coding |
| | | | transient receptor potential channels | |
| chr17 | 17677711 | NCRNA00034 | Smith-Magenis syndrome chromosome | ncRNA |
| | | | region, candidate 5 | |
| chr17 | 17677757 | NCRNA00034 | Smith-Magenis syndrome chromosome | ncRNA |
| | | | region, candidate 5 | |
| chr17 | 17677887 | NCRNA00034 | Smith-Magenis syndrome chromosome | ncRNA |
| | | | region, candidate 5 | |
| chr17 | 21286388 | IRK-2\|IRK2\|KCNJN1\|Kir2.2\|Kir2.2v\|hIRK\|hIRK1\| | potassium voltage-gated channel subfamily | protein-coding |
| | | hkir2.2x\|kcnj12x | member 12 | |
| chr17 | 21286444 | IRK-2\|IRK2\|KCNJN1\|Kir2.2\|Kir2.2v\|hIRK\|hIRK1\| | potassium voltage-gated channel subfamily | protein-coding |
| | | hkir2.2x\|kcnj12x | member 12 | |
| chr17 | 21286486 | IRK-2\|IRK2\|KCNJN1\|Kir2.2\|Kir2.2v\|hIRK\|hIRK1\| | potassium voltage-gated channel subfamily | protein-coding |
| | | hkir2.2x\|kcnj12x | member 12 | |
| chr17 | 21286510 | IRK-2\|IRK2\|KCNJN1\|Kir2.2\|Kir2.2v\|hIRK\|hIRK1\| | potassium voltage-gated channel subfamily | protein-coding |
| | | hkir2.2x\|kcnj12x | member 12 | |
| chr17 | 21286534 | IRK-2\|IRK2\|KCNJN1\|Kir2.2\|Kir2.2v\|hIRK\|hIRK1\| | potassium voltage-gated channel subfamily | protein-coding |
| | | hkir2.2x\|kcnj12x | member 12 | |
| chr17 | 21286708 | IRK-2\|IRK2\|KCNJN1\|Kir2.2\|Kir2.2v\|hIRK\|hIRK1\| | potassium voltage-gated channel subfamily | protein-coding |
| | | hkir2.2x\|kcnj12x | member 12 | |
| chr17 | 21286741 | IRK-2\|IRK2\|KCNJN1\|Kir2.2\|Kir2.2v\|hIRK\|hIRK1\| | potassium voltage-gated channel subfamily | protein-coding |
| | | hkir2.2x\|kcnj12x | member 12 | |
| chr17 | 21286747 | IRK-2\|IRK2\|KCNJN1\|Kir2.2\|Kir2.2v\|hIRK\|hIRK1\| | potassium voltage-gated channel subfamily | protein-coding |
| | | hkir2.2x\|kcnj12x | member 12 | |
| chr17 | 28299552 | — | EF-hand calcium binding domain 5 | protein-coding |
| chr17 | 28299565 | — | EF-hand calcium binding domain 5 | protein-coding |
| chr17 | 28299571 | — | EF-hand calcium binding domain 5 | protein-coding |
| chr17 | 28299598 | — | EF-hand calcium binding domain 5 | protein-coding |
| chr17 | 28299697 | — | EF-hand calcium binding domain 5 | protein-coding |
| chr17 | 71715903 | — | uncharacterized LOC100134391 | protein-coding |
| chr17 | 71715940 | — | uncharacterized LOC100134391 | protein-coding |
| chr17 | 71715947 | — | uncharacterized LOC100134391 | protein-coding |
| chr17 | 71715958 | — | uncharacterized LOC100134391 | protein-coding |
| chr17 | 71716005 | — | uncharacterized LOC100134391 | protein-coding |
| chr17 | 71743873 | — | uncharacterized LOC100134391 | protein-coding |
| chr17 | 71744668 | — | uncharacterized LOC100134391 | protein-coding |
| chr17 | 71744685 | — | uncharacterized LOC100134391 | protein-coding |
| chr17 | 71744723 | — | uncharacterized LOC100134391 | protein-coding |
| chr17 | 71744739 | — | uncharacterized LOC100134391 | protein-coding |
| chr17 | 78079606 | LYAG | glucosidase alpha, acid | protein-coding |
| chr17 | 78079613 | LYAG | glucosidase alpha, acid | protein-coding |
| chr17 | 78079629 | LYAG | glucosidase alpha, acid | protein-coding |
| chr17 | 78079641 | LYAG | glucosidase alpha, acid | protein-coding |
| chr17 | 78079653 | LYAG | glucosidase alpha, acid | protein-coding |
| chr17 | 78395359 | — | microRNA 4730 | ncRNA |
| chr17 | 78395376 | — | microRNA 4730 | ncRNA |
| chr17 | 80314127 | — | testis expressed 19 | protein-coding |
| chr17 | 80314154 | — | testis expressed 19 | protein-coding |
| chr17 | 80914812 | 3-Gn-T8\|B3GNT8\|BGnT-8\|beta-1\|beta3Gn- | UDP-GlcNAc:betaGal beta-1,3-N- | protein-coding |
| | | T8\|beta3GnTL1 | acetylglucosaminyltransferase like 1 | |
| chr17 | 80915007 | 3-Gn-T8\|B3GNT8\|BGnT-8\|beta-1\|beta3Gn- | UDP-GlcNAc:betaGal beta-1,3-N- | protein-coding |
| | | T8\|beta3GnTL1 | acetylglucosaminyltransferase like 1 | |
| chr17 | 80915042 | 3-Gn-T8\|B3GNT8\|BGnT-8\|beta-1\|beta3Gn- | UDP-GlcNAc:betaGal beta-1,3-N- | protein-coding |
| | | T8\|beta3GnTL1 | acetylglucosaminyltransferase like 1 | |
| chr17 | 80915160 | 3-Gn-T8\|B3GNT8\|BGnT-8\|beta-1\|beta3Gn- | UDP-GlcNAc:betaGal beta-1,3-N- | protein-coding |
| | | T8\|beta3GnTL1 | acetylglucosaminyltransferase like 1 | |
| chr17 | 80915190 | 3-Gn-T8\|B3GNT8\|BGnT-8\|beta-1\|beta3Gn- | UDP-GlcNAc:betaGal beta-1,3-N- | protein-coding |
| | | T8\|beta3GnTL1 | acetylglucosaminyltransferase like 1 | |
| chr17 | 80915238 | 3-Gn-T8\|B3GNT8\|BGnT-8\|beta-1\|beta3Gn- | UDP-GlcNAc:betaGal beta-1,3-N- | protein-coding |
| | | T8\|beta3GnTL1 | acetylglucosaminyltransferase like 1 | |
| chr17 | 80915248 | 3-Gn-T8\|B3GNT8\|BGnT-8\|beta-1\|beta3Gn- | UDP-GlcNAc:betaGal beta-1,3-N- | protein-coding |
| | | T8\|beta3GnTL1 | acetylglucosaminyltransferase like 1 | |
| chr17 | 80915270 | 3-Gn-T8\|B3GNT8\|BGnT-8\|beta-1\|beta3Gn- | UDP-GlcNAc:betaGal beta-1,3-N- | protein-coding |
| | | T8\|beta3GnTL1 | acetylglucosaminyltransferase like 1 | |
| chr17 | 80915289 | 3-Gn-T8\|B3GNT8\|BGnT-8\|beta-1\|beta3Gn- | UDP-GlcNAc:betaGal beta-1,3-N- | protein-coding |
| | | T8\|beta3GnTL1 | acetylglucosaminyltransferase like 1 | |
| chr17 | 80915308 | 3-Gn-T8\|B3GNT8\|BGnT-8\|beta-1\|beta3Gn- | UDP-GlcNAc:betaGal beta-1,3-N- | protein-coding |
| | | T8\|beta3GnTL1 | acetylglucosaminyltransferase like 1 | |
| chr18 | 8659649 | MTCL1-AS1\|MTCL1AS1 | gastric cancer associated transcript 2 | ncRNA |
| chr18 | 24348426 | LINC01092 | prostate cancer associated transcript 18 | ncRNA |
| chr18 | 25366256 | CD325\|CDHN\|CDw325\|NCAD | cadherin 2 | protein-coding |
| chr18 | 25366262 | CD325\|CDHN\|CDw325\|NCAD | cadherin 2 | protein-coding |
| chr18 | 25366300 | CD325\|CDHN\|CDw325\|NCAD | cadherin 2 | protein-coding |
| chr18 | 25366309 | CD325\|CDHN\|CDw325\|NCAD | cadherin 2 | protein-coding |
| chr18 | 25442175 | CD325\|CDHN\|CDw325\|NCAD | cadherin 2 | protein-coding |
| chr18 | 25442224 | CD325\|CDHN\|CDw325\|NCAD | cadherin 2 | protein-coding |
| chr18 | 25442243 | CD325\|CDHN\|CDw325\|NCAD | cadherin 2 | protein-coding |
| chr18 | 68166868 | — | long intergenic non-protein coding RNA | ncRNA |
| | | | 1910 | |
| chr18 | 68166878 | — | long intergenic non-protein coding RNA | ncRNA |
| | | | 1910 | |
| chr18 | 77320496 | — | uncharacterized LOC284240 | ncRNA |
| chr18 | 77320538 | — | uncharacterized LOC284240 | ncRNA |
| chr18 | 77320583 | — | uncharacterized LOC284240 | ncRNA |
| chr18 | 77320593 | — | uncharacterized LOC284240 | ncRNA |
| chr18 | 77320606 | — | uncharacterized LOC284240 | ncRNA |
| chr18 | 77320672 | — | uncharacterized LOC284240 | ncRNA |
| chr18 | 77320695 | — | uncharacterized LOC284240 | ncRNA |
| chr18 | 77320711 | — | uncharacterized LOC284240 | ncRNA |
| chr18 | 77320739 | — | uncharacterized LOC284240 | ncRNA |
| chr19 | 883734 | DRIP92\|THRAP5\|TRAP95 | mediator complex subunit 16 | protein-coding |
| chr19 | 883775 | DRIP92\|THRAP5\|TRAP95 | mediator complex subunit 16 | protein-coding |
| chr19 | 883830 | DRIP92\|THRAP5\|TRAP95 | mediator complex subunit 16 | protein-coding |
| chr19 | 883873 | DRIP92\|THRAP5\|TRAP95 | mediator complex subunit 16 | protein-coding |
| chr19 | 883885 | DRIP92\|THRAP5\|TRAP95 | mediator complex subunit 16 | protein-coding |
| chr19 | 883994 | DRIP92\|THRAP5\|TRAP95 | mediator complex subunit 16 | protein-coding |
| chr19 | 884032 | DRIP92\|THRAP5\|TRAP95 | mediator complex subunit 16 | protein-coding |
| chr19 | 884042 | DRIP92\|THRAP5\|TRAP95 | mediator complex subunit 16 | protein-coding |
| chr19 | 884057 | DRIP92\|THRAP5\|TRAP95 | mediator complex subunit 16 | protein-coding |
| chr19 | 884140 | DRIP92\|THRAP5\|TRAP95 | mediator complex subunit 16 | protein-coding |
| chr19 | 884147 | DRIP92\|THRAP5\|TRAP95 | mediator complex subunit 16 | protein-coding |
| chr19 | 884172 | DRIP92\|THRAP5\|TRAP95 | mediator complex subunit 16 | protein-coding |
| chr19 | 884233 | DRIP92\|THRAP5\|TRAP95 | mediator complex subunit 16 | protein-coding |
| chr19 | 884267 | DRIP92\|THRAP5\|TRAP95 | mediator complex subunit 16 | protein-coding |
| chr19 | 884346 | DRIP92\|THRAP5\|TRAP95 | mediator complex subunit 16 | protein-coding |
| chr19 | 2299058 | LERN2\|LRRN6B | leucine rich repeat and Ig domain | protein-coding |
| | | | containing 3 | |
| chr19 | 2299180 | LERN2\|LRRN6B | leucine rich repeat and Ig domain | protein-coding |
| | | | containing 3 | |
| chr19 | 2299218 | LERN2\|LRRN6B | leucine rich repeat and Ig domain | protein-coding |
| | | | containing 3 | |
| chr19 | 2299353 | LERN2\|LRRN6B | leucine rich repeat and Ig domain | protein-coding |
| | | | containing 3 | |
| chr19 | 2299390 | LERN2\|LRRN6B | leucine rich repeat and Ig domain | protein-coding |
| | | | containing 3 | |
| chr19 | 4566603 | SEM-SEMA-Y\|SEMA-VIB\|SEMAN\|semaZ | semaphorin 6B | protein-coding |
| chr19 | 4566636 | SEM-SEMA-Y\|SEMA-VIB\|SEMAN\|semaZ | semaphorin 6B | protein-coding |
| chr19 | 4566642 | SEM-SEMA-Y\|SEMA-VIB\|SEMAN\|semaZ | semaphorin 6B | protein-coding |
| chr19 | 4566653 | SEM-SEMA-Y\|SEMA-VIB\|SEMAN\|semaZ | semaphorin 6B | protein-coding |
| chr19 | 9994121 | NOE2\|NOELIN2\|NOELIN2_V1\|OlfC | olfactomedin 2 | protein-coding |
| chr19 | 9994137 | NOE2\|NOELIN2\|NOELIN2_V1\|OlfC | olfactomedin 2 | protein-coding |
| chr19 | 9994208 | NOE2\|NOELIN2\|NOELIN2_V1\|OlfC | olfactomedin 2 | protein-coding |
| chr19 | 9994219 | NOE2\|NOELIN2\|NOELIN2_V1\|OlfC | olfactomedin 2 | protein-coding |
| chr19 | 9994333 | NOE2\|NOELIN2\|NOELIN2_V1\|OlfC | olfactomedin 2 | protein-coding |
| chr19 | 9994349 | NOE2\|NOELIN2\|NOELIN2_V1\|OlfC | olfactomedin 2 | protein-coding |
| chr19 | 13108021 | CTF\|MRSHSS\|NF-1/X\|NF1-X\|NF1A\|SOTOS2 | nuclear factor IX | protein-coding |
| chr19 | 13108640 | CTF\|MRSHSS\|NF-1/X\|NF1-X\|NF1A\|SOTOS2 | nuclear factor IX | protein-coding |
| chr19 | 13112778 | CTF\|MRSHSS\|NF-1/X\|NF1-X\|NF1A\|SOTOS2 | nuclear factor IX | protein-coding |
| chr19 | 13616660 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13616810 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13616818 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13616834 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13616868 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13616879 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13616906 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13616911 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617010 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617018 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617024 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617029 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617058 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617065 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617070 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617082 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617090 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617128 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617140 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617157 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617163 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617169 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617176 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617194 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617208 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617218 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617233 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617241 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617250 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617265 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617288 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617302 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617315 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617321 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617328 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617336 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617364 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617380 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617419 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617465 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617483 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617503 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617512 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617517 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13617541 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13618356 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13618391 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 13700379 | APCA\|BI\|CACNL1A4\|CAV2.1\|EA2\|EIEE42\|FHM\| | calcium voltage-gated channel subunit | protein-coding |
| | | HPCA\|MHP\|MHP1\|SCA6 | alpha1 A | |
| chr19 | 18836689 | MAML2\|MECT1\|Mam-2\|TORC-1\|TORC1\|WAMTP1 | CREB regulated transcription coactivator 1 | protein-coding |
| chr19 | 18837043 | MAML2\|MECT1\|Mam-2\|TORC-1\|TORC1\|WAMTP1 | CREB regulated transcription coactivator 1 | protein-coding |
| chr19 | 18837091 | MAML2\|MECT1\|Mam-2\|TORC-1\|TORC1\|WAMTP1 | CREB regulated transcription coactivator 1 | protein-coding |
| chr19 | 18837098 | MAML2\|MECT1\|Mam-2\|TORC-1\|TORC1\|WAMTP1 | CREB regulated transcription coactivator 1 | protein-coding |
| chr19 | 18837105 | MAML2\|MECT1\|Mam-2\|TORC-1\|TORC1\|WAMTP1 | CREB regulated transcription coactivator 1 | protein-coding |
| chr19 | 34181556 | GALNAC4ST1\|GalNAc4ST\|PSS3 | carbohydrate sulfotransferase 8 | protein-coding |
| chr19 | 34181606 | GALNAC4ST1\|GalNAc4ST\|PSS3 | carbohydrate sulfotransferase 8 | protein-coding |
| chr19 | 34181636 | GALNAC4ST1\|GalNAc4ST\|PSS3 | carbohydrate sulfotransferase 8 | protein-coding |
| chr19 | 38527887 | — | small nucleolar RNA, C/D box 152 | snoRNA |
| chr19 | 38527913 | — | small nucleolar RNA, C/D box 152 | snoRNA |
| chr19 | 38527932 | — | small nucleolar RNA, C/D box 152 | snoRNA |
| chr19 | 38528016 | — | small nucleolar RNA, C/D box 152 | snoRNA |
| chr19 | 38528031 | — | small nucleolar RNA, C/D box 152 | snoRNA |
| chr19 | 38684235 | — | small nucleolar RNA, C/D box 152 | snoRNA |
| chr19 | 38684327 | BAF45b\|NEUD4\|neuro-d4 | double PHD fingers 1 | protein-coding |
| chr19 | 38684338 | BAF45b\|NEUD4\|neuro-d4 | double PHD fingers 1 | protein-coding |
| chr19 | 38684363 | BAF45b\|NEUD4\|neuro-d4 | double PHD fingers 1 | protein-coding |
| chr19 | 48699558 | C19orf68 | zinc finger SWIM-type containing 9 | protein-coding |
| chr19 | 48699578 | C19orf68 | zinc finger SWIM-type containing 9 | protein-coding |
| chr19 | 48699588 | C19orf68 | zinc finger SWIM-type containing 9 | protein-coding |
| chr19 | 48699615 | C19orf68 | zinc finger SWIM-type containing 9 | protein-coding |
| chr19 | 48699628 | C19orf68 | zinc finger SWIM-type containing 9 | protein-coding |
| chr19 | 48699667 | C19orf68 | zinc finger SWIM-type containing 9 | protein-coding |
| chr19 | 48699717 | C19orf68 | zinc finger SWIM-type containing 9 | protein-coding |
| chr19 | 55870392 | C19orf16 | family with sequence similarity 71 member | protein-coding |
| | | | E2 | |
| chr19 | 55870402 | C19orf16 | family with sequence similarity 71 member | protein-coding |
| | | | E2 | |
| chr19 | 55870407 | C19orf16 | family with sequence similarity 71 member | protein-coding |
| | | | E2 | |
| chr19 | 55870425 | C19orf16 | family with sequence similarity 71 member | protein-coding |
| | | | E2 | |
| chr19 | 55870439 | C19orf16 | family with sequence similarity 71 member | protein-coding |
| | | | E2 | |
| chr19 | 55870456 | C19orf16 | family with sequence similarity 71 member | protein-coding |
| | | | E2 | |
| chr19 | 55870462 | C19orf16 | family with sequence similarity 71 member | protein-coding |
| | | | E2 | |
| chr19 | 55870471 | C19orf16 | family with sequence similarity 71 member | protein-coding |
| | | | E2 | |
| chr19 | 55870886 | C19orf16 | family with sequence similarity 71 member | protein-coding |
| | | | E2 | |
| chr20 | 20180688 | C20orf26\|CaM-1P3\|dJ1002M8.3\|dJ1178H5.4 | cilia and flagella associated protein 61 | protein-coding |
| chr20 | 20180732 | C20orf26\|CaM-1P3\|dJ1002M8.3\|dJ1178H5.4 | cilia and flagella associated protein 61 | protein-coding |
| chr20 | 20188098 | C20orf26\|CaM-1P3\|dJ1002M8.3\|dJ1178H5.4 | cilia and flagella associated protein 61 | protein-coding |
| chr20 | 20188151 | C20orf26\|CaM-1P3\|dJ1002M8.3\|dJ1178H5.4 | cilia and flagella associated protein 61 | protein-coding |
| chr20 | 20206077 | IA-1\|IA1 | INSM transcriptional repressor 1 | protein-coding |
| chr20 | 20232312 | IA-1\|IA1 | INSM transcriptional repressor 1 | protein-coding |
| chr20 | 20232336 | IA-1\|IA1 | INSM transcriptional repressor 1 | protein-coding |
| chr20 | 20232350 | IA-1\|IA1 | INSM transcriptional repressor 1 | protein-coding |
| chr20 | 20232360 | IA-1\|IA1 | INSM transcriptional repressor 1 | protein-coding |
| chr20 | 20232370 | IA-1\|IA1 | INSM transcriptional repressor 1 | protein-coding |
| chr20 | 20232391 | IA-1\|IA1 | INSM transcriptional repressor 1 | protein-coding |
| chr20 | 20376947 | IA-1\|IA1 | INSM transcriptional repressor 1 | protein-coding |
| chr20 | 20376962 | IA-1\|IA1 | INSM transcriptional repressor 1 | protein-coding |
| chr20 | 20377074 | IA-1\|IA1 | INSM transcriptional repressor 1 | protein-coding |
| chr20 | 20377089 | IA-1\|IA1 | INSM transcriptional repressor 1 | protein-coding |
| chr20 | 20377110 | IA-1\|IA1 | INSM transcriptional repressor 1 | protein-coding |
| chr20 | 20377122 | IA-1\|IA1 | INSM transcriptional repressor 1 | protein-coding |
| chr20 | 20377130 | IA-1\|IA1 | INSM transcriptional repressor 1 | protein-coding |
| chr20 | 20377162 | IA-1\|IA1 | INSM transcriptional repressor 1 | protein-coding |
| chr20 | 20377169 | IA-1\|IA1 | INSM transcriptional repressor 1 | protein-coding |
| chr20 | 20377206 | IA-1\|IA1 | INSM transcriptional repressor 1 | protein-coding |
| chr20 | 20392139 | IA-1\|IA1 | INSM transcriptional repressor 1 | protein-coding |
| chr20 | 20392184 | IA-1\|IA1 | INSM transcriptional repressor 1 | protein-coding |
| chr20 | 20392215 | IA-1\|IA1 | INSM transcriptional repressor 1 | protein-coding |
| chr20 | 20392408 | IA-1\|IA1 | INSM transcriptional repressor 1 | protein-coding |
| chr20 | 20421295 | IA-1\|IA1 | INSM transcriptional repressor 1 | protein-coding |
| chr20 | 20421314 | IA-1\|IA1 | INSM transcriptional repressor 1 | protein-coding |
| chr20 | 20421345 | IA-1\|IA1 | INSM transcriptional repressor 1 | protein-coding |
| chr20 | 20421371 | IA-1\|IA1 | INSM transcriptional repressor 1 | protein-coding |
| chr20 | 20421396 | IA-1\|IA1 | INSM transcriptional repressor 1 | protein-coding |
| chr20 | 20421420 | IA-1\|IA1 | INSM transcriptional repressor 1 | protein-coding |
| chr20 | 20421459 | IA-1\|IA1 | INSM transcriptional repressor 1 | protein-coding |
| chr20 | 20421486 | IA-1\|IA1 | INSM transcriptional repressor 1 | protein-coding |
| chr20 | 31129481 | — | uncharacterized LOC101929698 | ncRNA |
| chr20 | 36923352 | BPIFD1\|rBPI | bactericidal permeability increasing protein | protein-coding |
| chr20 | 36923369 | BPIFD1\|rBPI | bactericidal permeability increasing protein | protein-coding |
| chr20 | 36923397 | BPIFD1\|rBPI | bactericidal permeability increasing protein | protein-coding |
| chr20 | 61013054 | C20orf151 | RBBP8 N-terminal like | protein-coding |
| chr20 | 61013094 | C20orf151 | RBBP8 N-terminal like | protein-coding |
| chr20 | 61013113 | C20orf151 | RBBP8 N-terminal like | protein-coding |
| chr20 | 61013148 | C20orf151 | RBBP8 N-terminal like | protein-coding |
| chr20 | 61013167 | C20orf151 | RBBP8 N-terminal like | protein-coding |
| chr20 | 61013187 | C20orf151 | RBBP8 N-terminal like | protein-coding |
| chr20 | 61013226 | C20orf151 | RBBP8 N-terminal like | protein-coding |
| chr20 | 61013242 | C20orf151 | RBBP8 N-terminal like | protein-coding |
| chr20 | 61013251 | C20orf151 | RBBP8 N-terminal like | protein-coding |
| chr20 | 61013263 | C20orf151 | RBBP8 N-terminal like | protein-coding |
| chr20 | 61013303 | C20orf151 | RBBP8 N-terminal like | protein-coding |
| chr20 | 61013339 | C20orf151 | RBBP8 N-terminal like | protein-coding |
| chr20 | 61013474 | C20orf151 | RBBP8 N-terminal like | protein-coding |
| chr20 | 61013515 | C20orf151 | RBBP8 N-terminal like | protein-coding |
| chr20 | 61020520 | C20orf151 | RBBP8 N-terminal like | protein-coding |
| chr20 | 61021747 | C20orf151 | RBBP8 N-terminal like | protein-coding |
| chr21 | 43442046 | C21orf121\|NCRNA00318\|PRED87 | ZNF295 antisense RNA 1 | ncRNA |
| chr21 | 43442055 | C21orf121\|NCRNA00318\|PRED87 | ZNF295 antisense RNA 1 | ncRNA |
| chr21 | 43442083 | C21orf121\|NCRNA00318\|PRED87 | ZNF295 antisense RNA 1 | ncRNA |
| chr21 | 43442252 | C21orf121\|NCRNA00318\|PRED87 | ZNF295 antisense RNA 1 | ncRNA |
| chr21 | 43442338 | C21orf121\|NCRNA00318\|PRED87 | ZNF295 antisense RNA 1 | ncRNA |
| chr21 | 43442384 | C21orf121\|NCRNA00318\|PRED87 | ZNF295 antisense RNA 1 | ncRNA |
| chr21 | 44836763 | MSK\|SIK\|SIK-1\|SIK1B\|SNF1LK | salt inducible kinase 1 | protein-coding |
| chr21 | 44836797 | MSK\|SIK\|SIK-1\|SIK1B\|SNF1LK | salt inducible kinase 1 | protein-coding |
| chr21 | 144836823 | MSK\|SIK\|SIK-1\|SIK1B\|SNF1LK | salt inducible kinase 1 | protein-coding |
| chr21 | 44836842 | MSK\|SIK\|SIK-1\|SIK1B\|SNF1LK | salt inducible kinase 1 | protein-coding |
| chr21 | 44836924 | MSK\|SIK\|SIK-1\|SIK1B\|SNF1LK | salt inducible kinase 1 | protein-coding |
| chr21 | 44836936 | MSK\|SIK\|SIK-1\|SIK1B\|SNF1LK | salt inducible kinase 1 | protein-coding |
| chr21 | 44836971 | MSK\|SIK\|SIK-1\|SIK1B\|SNF1LK | salt inducible kinase 1 | protein-coding |
| chr21 | 44837026 | MSK\|SIK\|SIK-1\|SIK1B\|SNF1LK | salt inducible kinase 1 | protein-coding |
| chr21 | 44837044 | MSK\|SIK\|SIK-1\|SIK1B\|SNF1LK | salt inducible kinase 1 | protein-coding |
| chr21 | 44837052 | MSK\|SIK\|SIK-1\|SIK1B\|SNF1LK | salt inducible kinase 1 | protein-coding |
| chr21 | 44837060 | MSK\|SIK\|SIK-1\|SIK1B\|SNF1LK | salt inducible kinase 1 | protein-coding |
| chr21 | 44837074 | MSK\|SIK\|SIK-1\|SIK1B\|SNF1LK | salt inducible kinase 1 | protein-coding |
| chr21 | 44837115 | MSK\|SIK\|SIK-1\|SIK1B\|SNF1LK | salt inducible kinase 1 | protein-coding |
| chr21 | 44837132 | MSK\|SIK\|SIK-1\|SIK1B\|SNF1LK | salt inducible kinase 1 | protein-coding |
| chr21 | 44837157 | MSK\|SIK\|SIK-1\|SIK1B\|SNF1LK | salt inducible kinase 1 | protein-coding |
| chr21 | 145653981 | B7-H2\|B7H2\|B7RP- | inducible T cell costimulator ligand | protein-coding |
| | | 1\|B7RP1\|B7h\|CD275\|GL50\|ICOS-L\|ICOSL\|LICOS | | |
| chr21 | 45654156 | B7-H2\|B7H2\|B7RP- | inducible T cell costimulator ligand | protein-coding |
| | | 1\|B7RP1\|B7h\|CD275\|GL50\|ICOS-L\|ICOSL\|LICOS | | |
| chr22 | 47444997 | — | TBC1D22A antisense RNA 1 | ncRNA |
| chr22 | 47445037 | — | TBC1D22A antisense RNA 1 | ncRNA |
| chr22 | 47447406 | — | TBC1D22A antisense RNA 1 | ncRNA |
| chr22 | 47447603 | — | TBC1D22A antisense RNA 1 | ncRNA |
| chrX | 104458760 | — | testis expressed 13A | protein-coding |
| chrX | 104692650 | — | testis expressed 13A | protein-coding |
| chrX | 104804571 | NESK | Nik related kinase | protein-coding |
| chrX | 104891135 | NESK | Nik related kinase | protein-coding |
| chrX | 105016634 | NESK | Nik related kinase | protein-coding |
| chrX | 105016672 | NESK | Nik related kinase | protein-coding |
| chrX | 128926432 | 753P9\|CXorf9\|HACS2\|SH3D6C\|SLY | SAM and SH3 domain containing 3 | protein-coding |
| chr2 | 209103955 | HEL-216\|HEL-S-26\|IDCD\|IDH\|IDP\|IDPC\|PICD | isocitrate dehydrogenase (NADP(+)) 1, | protein-coding |
| | | | cytosolic | |
| chr4 | 106155150 | KIAA1546\|MDS | tet methylcytosine dioxygenase 2 | protein-coding |
| chr4 | 106155913 | KIAA1546\|MDS | tet methylcytosine dioxygenase 2 | protein-coding |
| chr4 | 106156460 | KIAA1546\|MDS | tet methylcytosine dioxygenase 2 | protein-coding |
| chr4 | 106156742 | KIAA1546\|MDS | tet methylcytosine dioxygenase 2 | protein-coding |
| chr4 | 106157415 | KIAA1546\|MDS | tet methylcytosine dioxygenase 2 | protein-coding |
| chr4 | 106164901 | KIAA1546\|MDS | tet methylcytosine dioxygenase 2 | protein-coding |
| chr4 | 106196236 | — | TET2 antisense RNA 1 | ncRNA |
| chr7 | 33732332 | CRIM3\|CV-2\|CV2 | BMP binding endothelial regulator | protein-coding |
| chr7 | 140453119 | — | NDUFB2 antisense RNA 1 | ncRNA |
| chr7 | 140481387 | — | NDUFB2 antisense RNA 1 | ncRNA |
| chr7 | 140481426 | — | NDUFB2 antisense RNA 1 | ncRNA |
| chr7 | 140500421 | — | NDUFB2 antisense RNA 1 | ncRNA |
| chr7 | 140507748 | — | NDUFB2 antisense RNA 1 | ncRNA |
| chr7 | 140507954 | — | NDUFB2 antisense RNA 1 | ncRNA |
| chr7 | 140624441 | B-RAF1B-raf\|BRAF 1NS7\|R AFB1 | B-Raf proto-oncogene, serine/threonine | protein-coding |
| | | | kinase | |
| chr14 | 38061176 | HNF3A\|TCF3A | forkhead box A1 | protein-coding |
| chr14 | 38061319 | HNF3A\|TCF3A | forkhead box A1 | protein-coding |
| chr14 | 38061830 | HNF3A\|TCF3A | forkhead box A1 | protein-coding |
| chr17 | 7572948 | BCC7\|BMFS5\|LFS1\|P53\|TRP53 | tumor protein p53 | protein-coding |
| chr17 | 7573967 | BCC7\|BMFS5\|LFS1\|P53\|TRP53 | tumor protein p53 | protein-coding |
| chr17 | 7576524 | BCC7\|BMFS5\|LFS1\|P53\|TRP53 | tumor protein p53 | protein-coding |
| chr17 | 7576838 | BCC7\|BMFS5\|LFS1\|P53\|TRP53 | tumor protein p53 | protein-coding |
| chr17 | 7577020 | BCC7\|BMFS5\|LFS1\|P53\|TRP53 | tumor protein p53 | protein-coding |
| chr17 | 7577483 | BCC7\|BMFS5\|LFS1\|P53\|TRP53 | tumor protein p53 | protein-coding |
| chr17 | 7578161 | BCC7\|BMFS5\|LFS1\|P53\|TRP53 | tumor protein p53 | protein-coding |
| chr17 | 7578373 | BCC7\|BMFS5\|LFS1\|P53\|TRP53 | tumor protein p53 | protein-coding |
| chr17 | 7579297 | BCC7\|BMFS5\|LFS1\|P53\|TRP53 | tumor protein p53 | protein-coding |
| chr17 | 7579393 | BCC7\|BMFS5\|LFS1\|P53[IRP53 | tumor protein p53 | protein-coding |
| chr17 | 7579506 | BCC7\|BMFS5\|LFS1\|P53\|TRP53 | tumor protein p53 | protein-coding |
| chr17 | 7579701 | BCC7\|BMFS5\|LFS1\|P53\|TRP53 | tumor protein p53 | protein-coding |
| chr17 | 7579846 | BCC7\|BMFS5\|LFS1\|P53\|TRP53 | tumor protein p53 | protein-coding |
| chr17 | 47696409 | BTBD32\|TEF2 | speckle type BTB/POZ protein | protein-coding |
| chr17 | 47696673 | BTBD32\|TEF2 | speckle type BTB/POZ protein | protein-coding |
| chr20 | 31375142 | ICF\|ICF1\|M.HsaIIIB | DNA methyltransferase 3 beta | protein-coding |
| chr20 | 31380384 | ICF\|ICF1\|M.HsaIIIB | DNA methyltransferase 3 beta | protein-coding |
| chr20 | 31381300 | ICF\|ICF1\|M.HsaIIIB | DNA methyltransferase 3 beta | protein-coding |
| chr20 | 31386255 | ICF\|ICF1\|M.HsaIIIB | DNA methyltransferase 3 beta | protein-coding |
| chr20 | 31388050 | EB1 | microtubule associated protein RP/EB | protein-coding |
| | | | family member 1 | |
| chr20 | 31389120 | EB1 | microtubule associated protein RP/EB | protein-coding |
| | | | family member 1 | |
| chr20 | 31393185 | EB1 | microtubule associated protein RP/EB | protein-coding |
| | | | family member 1 | |
| chr20 | 31395662 | EB1 | microtubule associated protein RP/EB | protein-coding |
| | | | family member 1 | |
| chrX | 66121000 | EDA-A2R\|EDAA2R\|TNFRSF27\|XEDAR | ectodysplasin A2 receptor | protein-coding |
| chrX | 66766332 | AIS\|AR8\|DHTR\|HUMARA\|HYSP1\|KD\|NR3C4\| | androgen receptor | protein-coding |
| | | SBMA\|SMAX1\|IFM | | |
| chrX | 66931448 | AIS\|AR8\|DHTR\|HUMARA\|HYSP1\|KD\|NR3C4\| | androgen receptor | protein-coding |
| | | SBMA\|SMAX1\|IFM | | |
| chrX | 66937357 | AIS\|AR8\|DHTR\|HUMARA\|HYSP1\|KD\|NR3C4\| | androgen receptor | protein-coding |
| | | SBMA\|SMAX1\|IFM | | |
| chrX | 66943528 | AIS\|AR8\|DHTR\|HUMARA\|HYSP1\|KD\|NR3C4\| | androgen receptor | protein-coding |
| | | SBMA\|SMAX1\|IFM | | |
| chr2 | 25452399 | DNMT3A2\|M.HsaIIIA\|TBRS | DNA methyltransferase 3 alpha | protein-coding |
| chr2 | 25452969 | DNMT3A2\|M.HsaIIIA\|TBRS | DNA methyltransferase 3 alpha | protein-coding |
| chr2 | 25453099 | DNMT3A2\|M.HsaIIIA\|TBRS | DNA methyltransferase 3 alpha | protein-coding |
| chr2 | 25453105 | DNMT3A2\|M.HsaIIIA\|TBRS | DNA methyltransferase 3 alpha | protein-coding |
| chr2 | 25453799 | DNMT3A2\|M.HsaIIIA\|TBRS | DNA methyltransferase 3 alpha | protein-coding |
| chr2 | 25453898 | DNMT3A2\|M.HsaIIIA\|TBRS | DNA methyltransferase 3 alpha | protein-coding |
| chr2 | 25453899 | DNMT3A2\|M.HsaIIIA\|TBRS | DNA methyltransferase 3 alpha | protein-coding |
| chr2 | 25453913 | DNMT3A2\|M.HsaIIIA\|TBRS | DNA methyltransferase 3 alpha | protein-coding |
| chr2 | 25499982 | DNMT3A2\|M.HsaIIIA\|TBRS | DNA methyltransferase 3 alpha | protein-coding |
| chr2 | 25500271 | DNMT3A2\|M.HsaIIIA\|TBRS | DNA methyltransferase 3 alpha | protein-coding |
| chr2 | 25500272 | DNMT3A2\|M.HsaIIIA\|TBRS | DNA methyltransferase 3 alpha | protein-coding |
| chr2 | 239147428 | C-HAIRY1\|HES-6\|bHLHb41\|bHLHc23 | hes family bHLH transcription factor 6 | protein-coding |
| chr2 | 239147440 | C-HAIRY1\|HES-6\|bHLHb41\|bHLHc23 | hes family bHLH transcription factor 6 | protein-coding |
| chr2 | 239147441 | C-HAIRY1\|HES-6\|bHLHb41\|bHLHc23 | hes family bHLH transcription factor 6 | protein-coding |
| chr2 | 239147485 | C-HAIRY1\|HES-6\|bHLHb41\|bHLHc23 | hes family bHLH transcription factor 6 | protein-coding |
| chr2 | 239147486 | C-HAIRY1\|HES-6\|bHLHb41\|bHLHc23 | hes family bHLH transcription factor 6 | protein-coding |
| chr2 | 239147522 | C-HAIRY1\|HES-6\|bHLHb41\|bHLHc23 | hes family bHLH transcription factor 6 | protein-coding |
| chr2 | 239147534 | C-HAIRY1\|HES-6\|bHLHb41\|bHLHc23 | hes family bHLH transcription factor 6 | protein-coding |
| chr2 | 239147589 | C-HAIRY1\|HES-6\|bHLHb41\|bHLHc23 | hes family bHLH transcription factor 6 | protein-coding |
| chr2 | 239147601 | C-HAIRY1\|HES-6\|bHLHb41\|bHLHc23 | hes family bHLH transcription factor 6 | protein-coding |
| chr2 | 239147614 | C-HAIRY1\|HES-6\|bHLHb41\|bHLHc23 | hes family bHLH transcription factor 6 | protein-coding |
| chr2 | 239147637 | C-HAIRY1\|HES-6\|bHLHb41\|bHLHc23 | hes family bHLH transcription factor 6 | protein-coding |
| chr2 | 239147649 | C-HAIRY1\|HES-6\|bHLHb41\|bHLHc23 | hes family bHLH transcription factor 6 | protein-coding |
| chr2 | 239147665 | C-HAIRY1\|HES-6\|bHLHb41\|bHLHc23 | hes family bHLH transcription factor 6 | protein-coding |
| chr2 | 239149894 | C-HAIRY1\|HES-6\|bHLHb41\|bHLHc23 | hes family bHLH transcription factor 6 | protein-coding |
| chr2 | 239149895 | C-HAIRY1\|HES-6\|bHLHb41\|bHLHc23 | hes family bHLH transcription factor 6 | protein-coding |
| chr3 | 12838468 | TIP120B\|Tp120b | cullin associated and neddylation | protein-coding |
| | | | dissociated 2 (putative) | |
| chr3 | 50660608 | 3PK\|MAPKAP-K3\|MAPKAP3\|MAPKAPK- | MAPK activated protein kinase 3 | protein-coding |
| | | 3\|MDPT3\|MK-3 | | |
| chr3 | 50670155 | 3PK\|MAPKAP-K3\|MAPKAP3\|MAPKAPK- | MAPK activated protein kinase 3 | protein-coding |
| | | 3\|MDPT3\|MK-3 | | |
| chr3 | 128202781 | DCML\|IMD21\|MONOMAC\|NFE1B | GATA binding protein 2 | protein-coding |
| chr3 | 128202787 | DCML\|IMD21\|MONOMAC\|NFE1B | GATA binding protein 2 | protein-coding |
| chr3 | 128202792 | DCML\|IMD21\|MONOMAC\|NFE1B | GATA binding protein 2 | protein-coding |
| chr3 | 128202795 | DCML\|IMD21\|MONOMAC\|NFE1B | GATA binding protein 2 | protein-coding |
| chr3 | 128202796 | DCML\|IMD21\|MONOMAC\|NFE1B | GATA binding protein 2 | protein-coding |
| chr3 | 128202884 | DCML\|IMD21\|MONOMAC\|NFE1B | GATA binding protein 2 | protein-coding |
| chr3 | 128202894 | DCML\|IMD21\|MONOMAC\|NFE1B | GATA binding protein 2 | protein-coding |
| chr3 | 128202895 | DCML\|IMD21\|MONOMAC\|NFE1B | GATA binding protein 2 | protein-coding |
| chr3 | 128202927 | DCML\|IMD21\|MONOMAC\|NFE1B | GATA binding protein 2 | protein-coding |
| chr3 | 128202928 | DCML\|IMD21\|MONOMAC\|NFE1B | GATA binding protein 2 | protein-coding |
| chr3 | 128205157 | DCML\|IMD21\|MONOMAC\|NFE1B | GATA binding protein 2 | protein-coding |
| chr3 | 128205179 | DCML\|IMD21\|MONOMAC\|NFE1B | GATA binding protein 2 | protein-coding |
| chr3 | 128205180 | DCML\|IMD21\|MONOMAC\|NFE1B | GATA binding protein 2 | protein-coding |
| chr3 | 128205195 | DCML\|IMD21\|MONOMAC\|NFE1B | GATA binding protein 2 | protein-coding |
| chr3 | 128205196 | DCML\|IMD21\|MONOMAC\|NFE1B | GATA binding protein 2 | protein-coding |
| chr3 | 128205210 | DCML\|IMD21\|MONOMAC\|NFE1B | GATA binding protein 2 | protein-coding |
| chr3 | 128210723 | DCML\|IMD21\|MONOMAC\|NFE1B | GATA binding protein 2 | protein-coding |
| chr3 | 128210727 | DCML\|IMD21\|MONOMAC\|NFE1B | GATA binding protein 2 | protein-coding |
| chr3 | 128212207 | DCML\|IMD21\|MONOMAC\|NFE1B | GATA binding protein 2 | protein-coding |
| chr3 | 185817004 | ERM | ETS variant 5 | protein-coding |
| chr4 | 16084875 | | | |
| chr4 | 16084881 | | | |
| chr4 | 16084898 | | | |
| chr4 | 16085260 | AC133\|CD133\|CORD12\|MCDR2\|MSTP061\|PROML1\| | prominin 1 | protein-coding |
| | | RP41\|STGD4 | | |
| chr4 | 16085380 | AC133\|CD133\|CORD12\|MCDR2\|MSTP061\|PROML1\| | prominin 1 | protein-coding |
| | | RP41\|STGD4 | | |
| chr4 | 16085396 | AC133\|CD133\|CORD12\|MCDR2\|MSTP061\|PROML1\| | prominin 1 | protein-coding |
| | | RP41\|STGD4 | | |
| chr4 | 16085398 | AC133\|CD133\|CORD12\|MCDR2\|MSTP061\|PROML1\|R | prominin 1 | protein-coding |
| | | P41\|STGD4 | | |
| chr4 | 16085401 | AC133\|CD133\|CORD12\|MCDR2\|MSTP061\|PROML1\| | prominin 1 | protein-coding |
| | | RP41\|STGD4 | | |
| chr5 | 172661872 | CHNG5\|CSX\|CSX1\|HLHS2\|NKX2.5\|NKX2E\|NKX4- | NK2 homeobox 5 | protein-coding |
| | | 1\|VSD3 | | |
| chr5 | 172661874 | CHNG5\|CSX\|CSX1\|HLHS2\|NKX2.5\|NKX2E\|NKX4- | NK2 homeobox 5 | protein-coding |
| | | 1\|VSD3 | | |
| chr6 | 34522044 | PDEF\|bA375E1.3 | SAM pointed domain containing ETS | protein-coding |
| | | | transcription factor | |
| chr6 | 34523223 | PDEF\|bA375E1.3 | SAM pointed domain containing ETS | protein-coding |
| | | | transcription factor | |
| chr8 | 11561726 | ASD2\|TACHD\|TOF\|VSD1 | GATA binding protein 4 | protein-coding |
| chr8 | 11565855 | ASD2\|TACHD\|TOF\|VSD1 | GATA binding protein 4 | protein-coding |
| chr8 | 11566229 | ASD2\|TACHD\|TOF\|VSD1 | GATA binding protein 4 | protein-coding |
| chr8 | 11583613 | ASD2\|TACHD\|TOF\|VSD1 | GATA binding protein 4 | protein-coding |
| chr8 | 11583614 | ASD2\|TACHD\|TOF\|VSD1 | GATA binding protein 4 | protein-coding |
| chr9 | 126775063 | LH2\|hLhx2 | LIM homeobox 2 | protein-coding |
| chr9 | 126775082 | LH2\|hLhx2 | LIM homeobox 2 | protein-coding |
| chr9 | 126776013 | LH2\|hLhx2 | LIM homeobox 2 | protein-coding |
| chr9 | 126776231 | LH2\|hLhx2 | LIM homeobox 2 | protein-coding |
| chr9 | 139437635 | AOS5\|AOVD1\|TAN1\|hN1 | notch receptor 1 | protein-coding |
| chr9 | 139437679 | AOS5\|AOVD1\|TAN1\|hN1 | notch receptor 1 | protein-coding |
| chr10 | 23481414 | PACA\|PAGEN2\|PTF1-p48\|bHLHa29\|p48 | pancreas associated transcription factor 1a | protein-coding |
| chr10 | 23481415 | PACA\|PAGEN2\|PTF1-p48\|bHLHa29\|p48 | pancreas associated transcription factor 1a | protein-coding |
| chr10 | 23481417 | PACA\|PAGEN2\|PTF1-p48\|bHLHa29\|p48 | pancreas associated transcription factor 1a | protein-coding |
| chr10 | 23481445 | PACA\|PAGEN2\|PTF1-p48\|bHLHa29\|p48 | pancreas associated transcription factor 1a | protein-coding |
| chr10 | 23481447 | PACA\|PAGEN2\|PTF1-p48\|bHLHa29\|p48 | pancreas associated transcription factor 1a | protein-coding |
| chr10 | 23481448 | PACA\|PAGEN2\|PTF1-p48\|bHLHa29\|p48 | pancreas associated transcription factor 1a | protein-coding |
| chr10 | 23481451 | PACA\|PAGEN2\|PTF1-p48\|bHLHa29\|p48 | pancreas associated transcription factor 1a | protein-coding |
| chr10 | 23481461 | PACA\|PAGEN2\|PTF1-p48\|bHLHa29\|p48 | pancreas associated transcription factor 1a | protein-coding |
| chr10 | 23481462 | PACA\|PAGEN2\|PTF1-p48\|bHLHa29\|p48 | pancreas associated transcription factor 1a | protein-coding |
| chr10 | 23481463 | PACA\|PAGEN2\|PTF1-p48\|bHLHa29\|p48 | pancreas associated transcription factor 1a | protein-coding |
| chr10 | 56699066 | CDHR15\|DFNB23\|USH1F | protocadherin related 15 | protein-coding |
| chr10 | 56699077 | CDHR15\|DFNB23\|USH1F | protocadherin related 15 | protein-coding |
| chr10 | 57391015 | CDHR15\|DFNB23\|USH1F | protocadherin related 15 | protein-coding |
| chr11 | 69451450 | BCL1\|D11S287E\|PRAD1\|U21B31 | cyclin D1 | protein-coding |
| chr12 | 103344993 | ASH1\|HASH1\|MASH1\|bHLHa46 | achaete-scute family bHLH transcription | protein-coding |
| | | | factor 1 | |
| chr12 | 103344998 | ASH1\|HASH1\|MASH1\|bHLHa46 | achaete-scute family bHLH transcription | protein-coding |
| | | | factor 1 | |
| chr12 | 103345006 | ASH1\|HASH1\|MASH1\|bHLHa46 | achaete-scute family bHLH transcription | protein-coding |
| | | | factor 1 | |
| chr12 | 103345029 | ASH1\|HASH1\|MASH1\|bHLHa46 | achaete-scute family bHLH transcription | protein-coding |
| | | | factor 1 | |
| chr12 | 103345033 | ASH1\|HASH1\|MASH1\|bHLHa46 | achaete-scute family bHLH transcription | protein-coding |
| | | | factor 1 | |
| chr14 | 51560040 | RNF91\|SPRING | tripartite motif containing 9 | protein-coding |
| chr14 | 51560162 | RNF91\|SPRING | tripartite motif containing 9 | protein-coding |
| chr14 | 51560191 | RNF91\|SPRING | tripartite motif containing 9 | protein-coding |
| chr11 | 51560193 | RNF91\|SPRING | tripartite motif containing 9 | protein-coding |
| chr14 | 51560208 | RNF91\|SPRING | tripartite motif containing 9 | protein-coding |
| chr14 | 51560259 | RNF91\|SPRING | tripartite motif containing 9 | protein-coding |
| chr14 | 51560261 | RNF91\|SPRING | tripartite motif containing 9 | protein-coding |
| chr14 | 51560275 | RNF91\|SPRING | tripartite motif containing 9 | protein-coding |
| chr14 | 65409361 | GI-GPx\|GPRP\|GPRP-2\|GPx-2\|GPx-GI\|GSHPX- | glutathione peroxidase 2 | protein-coding |
| | | GI\|GSHPx-2 | | |
| chr14 | 93385930 | CGA | chromogranin A | protein-coding |
| chr14 | 93423499 | CGA | chromogranin A | protein-coding |
| chr14 | 93423555 | CGA | chromogranin A | protein-coding |
| chr14 | 93425158 | CGA | chromogranin A | protein-coding |
| chr14 | 93425189 | CGA | chromogranin A | protein-coding |
| chr14 | 93425207 | CGA | chromogranin A | protein-coding |
| chr14 | 93425289 | CGA | chromogranin A | protein-coding |
| chr14 | 93425328 | CGA | chromogranin A | protein-coding |
| chr14 | 93425517 | CGA | chromogranin A | protein-coding |
| chr14 | 93425684 | CGA | chromogranin A | protein-coding |
| chr14 | 93425685 | CGA | chromogranin A | protein-coding |
| chr14 | 93425738 | CGA | chromogranin A | protein-coding |
| chr14 | 93426226 | CGA | chromogranin A | protein-coding |
| chr14 | 93426244 | CGA | chromogranin A | protein-coding |
| chr14 | 93426333 | CGA | chromogranin A | protein-coding |
| chr14 | 93426378 | CGA | chromogranin A | protein-coding |
| chr14 | 93426872 | CGA | chromogranin A | protein-coding |
| chr14 | 93426913 | CGA | chromogranin A | protein-coding |
| chr14 | 93426914 | CGA | chromogranin A | protein-coding |
| chr14 | 93426959 | CGA | chromogranin A | protein-coding |
| chr14 | 93439910 | CGA | chromogranin A | protein-coding |
| chr18 | 31323189 | BRPS\|KIAA1713 | ASXL transcriptional regulator 3 | protein-coding |
| chr18 | 31323202 | BRPS\|KIAA1713 | ASXL transcriptional regulator 3 | protein-coding |
| chr18 | 53175324 | E2-2\|FECD3\|ITF-2\|ITF2\|PTHS\|SEF-2\|SEF2\|SEF2- | transcription factor 4 | protein-coding |
| | | 1\|SEF2-1A\|SEF2-1B\|SEF2-1D\|TCF-4\|bHLHb19 | | |
| chr18 | 53305364 | E2-2\|FECD3\|ITF-2\|ITF2\|PTHS\|SEF-2\|SEF2\|SEF2- | transcription factor 4 | protein-coding |
| | | 1\|SEF2-1A\|SEF2-1B\|SEF2-1D\|TCF-4\|bHLHb19 | | |
| chr18 | 53305490 | E2-2\|FECD3\|ITF-2\|ITF2\|PTHS\|SEF-2\|SEF2\|SEF2- | transcription factor 4 | protein-coding |
| | | 1\|SEF2-1A\|SEF2-1B\|SEF2-1D\|TCF-4\|bHLHb19 | | |
| chr19 | 11086495 | BAF190\|BAF190A\|BRG1\|CSS4\|MRD16\|RTPS2\|SNF2\| | SWI/SNF related, matrix associated, actin | protein-coding |
| | | SNF2L4\|SNF2LB\|SWI2\|hSNF2b | dependent regulator of chromatin, | |
| | | | subfamily a, member 4 | |
| chr19 | 39993532 | SCDO1 | delta like canonical Notch ligand 3 | protein-coding |
| chr19 | 39993551 | SCDO1 | delta like canonical Notch ligand 3 | protein-coding |
| chr19 | 39993567 | SCDO1 | delta like canonical Notch ligand 3 | protein-coding |
| chr19 | 39993606 | SCDO1 | delta like canonical Notch ligand 3 | protein-coding |
| chr19 | 42618585 | OCT2\|OTF2\|Oct-2 | POU class 2 homeobox 2 | protein-coding |
| chr19 | 42618760 | OCT2\|OTF2\|Oct-2 | POU class 2 homeobox 2 | protein-coding |
| chr19 | 42618787 | OCT2\|OTF2\|Oct-2 | POU class 2 homeobox 2 | protein-coding |
| chr19 | 42618789 | OCT2\|OTF2\|Oct-2 | POU class 2 homeobox 2 | protein-coding |
| chr19 | 42618823 | OCT2\|OTF2\|Oct-2 | POU class 2 homeobox 2 | protein-coding |
| chr20 | 20232309 | IA-1\|IA1 | INSM transcriptional repressor 1 | protein-coding |
| chr20 | 20232319 | IA-1\|IA1 | INSM transcriptional repressor 1 | protein-coding |
| chr20 | 20232333 | IA-1\|IA1 | INSM transcriptional repressor 1 | protein-coding |
| chr20 | 20232347 | IA-1\|IA1 | INSM transcriptional repressor 1 | protein-coding |
| chr20 | 20232348 | IA-1\|IA1 | INSM transcriptional repressor 1 | protein-coding |
| chr20 | 20232351 | IA-1\|IA1 | INSM transcriptional repressor 1 | protein-coding |
| chr20 | 20232352 | IA-1\|IA1 | INSM transcriptional repressor 1 | protein-coding |
| chr20 | 20232357 | IA-1\|IA1 | INSM transcriptional repressor 1 | protein-coding |
| chr20 | 20232358 | IA-1\|IA1 | INSM transcriptional repressor 1 | protein-coding |
| chr20 | 20232367 | IA-1\|IA1 | INSM transcriptional repressor 1 | protein-coding |
| chr20 | 20232388 | IA-1\|IA1 | INSM transcriptional repressor 1 | protein-coding |
| chr20 | 20266415 | IA-1\|IA1 | INSM transcriptional repressor 1 | protein-coding |
| chr20 | 20376954 | IA-1\|IA1 | INSM transcriptional repressor 1 | protein-coding |
| chr20 | 20376969 | IA-1\|IA1 | INSM transcriptional repressor 1 | protein-coding |
| chr20 | 20377086 | IA-1\|IA1 | INSM transcriptional repressor 1 | protein-coding |
| chr20 | 20377158 | IA-1\|IA1 | INSM transcriptional repressor 1 | protein-coding |
| chr20 | 20377165 | IA-1\|IA1 | INSM transcriptional repressor 1 | protein-coding |
| chr20 | 20421292 | IA-1\|IA1 | INSM transcriptional repressor 1 | protein-coding |
| chr20 | 20421311 | IA-1\|IA1 | INSM transcriptional repressor 1 | protein-coding |
| chr20 | 20421456 | IA-1\|IA1 | INSM transcriptional repressor 1 | protein-coding |
| chr20 | 20421483 | IA-1\|IA1 | INSM transcriptional repressor 1 | protein-coding |
| chr20 | 20433160 | IA-1\|IA1 | INSM transcriptional repressor 1 | protein-coding |
| chr20 | 20433163 | IA-1\|IA1 | INSM transcriptional repressor 1 | protein-coding |
| chr20 | 20433166 | IA-1\|IA1 | INSM transcriptional repressor 1 | protein-coding |
| chr20 | 20433198 | IA-1\|IA1 | INSM transcriptional repressor 1 | protein-coding |
| chr20 | 20433208 | IA-1\|IA1 | INSM transcriptional repressor 1 | protein-coding |
| chr20 | 20433248 | IA-1\|IA1 | INSM transcriptional repressor 1 | protein-coding |
| chr20 | 20433256 | IA-1\|IA1 | INSM transcriptional repressor 1 | protein-coding |
| chr20 | 20433263 | IA-1\|IA1 | INSM transcriptional repressor 1 | protein-coding |
| chr20 | 20433284 | IA-1\|IA1 | INSM transcriptional repressor 1 | protein-coding |
| chr20 | 20433293 | IA-1\|IA1 | INSM transcriptional repressor 1 | protein-coding |
| chr20 | 21490704 | NKX2.2\|NKX2B | NK2 homeobox 2 | protein-coding |
| chr20 | 21490799 | NKX2.2\|NKX2B | NK2 homeobox 2 | protein-coding |
| chr20 | 21490807 | NKX2.2\|NKX2B | NK2 homeobox 2 | protein-coding |
| chr20 | 21494139 | NKX2.2\|NKX2B | NK2 homeobox 2 | protein-coding |
| chr20 | 21494141 | NKX2.2\|NKX2B | NK2 homeobox 2 | protein-coding |
| chr20 | 21494237 | NKX2.2\|NKX2B | NK2 homeobox 2 | protein-coding |
| chr21 | 22564429 | NCAM21 | neural cell adhesion molecule 2 | protein-coding |
| chr21 | 42850711 | PP9284\|PRSS10 | transmembrane serine protease 2 | protein-coding |
| chr21 | 42860322 | PP9284\|PRSS10 | transmembrane serine protease 2 | protein-coding |
| chr21 | 42860341 | PP9284\|PRSS10 | transmembrane serine protease 2 | protein-coding |
| chr21 | 42880610 | PP9284\|PRSS10 | transmembrane serine protease 2 | protein-coding |
| chr21 | 42880614 | PP9284\|PRSS10 | transmembrane serine protease 2 | protein-coding |
| chr21 | 42880659 | PP9284\|PRSS10 | transmembrane serine protease 2 | protein-coding |
| chr21 | 42880668 | PP9284\|PRSS10 | transmembrane serine protease 2 | protein-coding |
| chr1 | 3052478 | — | microRNA 4251 | ncRNA |
| chr1 | 26735081 | CSDD1\|LIN-28\|LIN28\|ZCCHC1\|lin-28A | lin-28 homolog A | protein-coding |
| chr1 | 26735241 | CSDD1\|LIN-28\|LIN28\|ZCCHC1\|lin-28A | lin-28 homolog A | protein-coding |
| chr1 | 26735243 | CSDD1\|LIN-28\|LIN28\|ZCCHC1\|lin-28A | lin-28 homolog A | protein-coding |
| chr1 | 26735251 | CSDD1\|LIN-28\|LIN28\|ZCCHC1\|lin-28A | lin-28 homolog A | protein-coding |
| chr1 | 65721361 | DJC6\|PARK19 | DnaJ heat shock protein family (Hsp40) | protein-coding |
| | | | member C6 | |
| chr1 | 229195756 | — | microRNA 4454 | ncRNA |
| chr2 | 23807028 | KBTBD9 | kelch like family member 29 | protein-coding |
| chr2 | 171679652 | CPSQ1\|GAD\|SCP | glutamate decarboxylase 1 | protein-coding |
| chr2 | 236877206 | — | gastrulation brain homeobox 2 | protein-coding |
| chr2 | 242549633 | CGI-36\|Nb(III)\|PP238 | THAP domain containing 4 | protein-coding |
| chr2 | 242549635 | CGI-36\|Nb(III)\|PP238 | THAP domain containing 4 | protein-coding |
| chr2 | 242549638 | CGI-36\|Nb(III)\|PP238 | THAP domain containing 4 | protein-coding |
| chr2 | 242549659 | CGI-36\|Nb(III)\|PP238 | THAP domain containing 4 | protein-coding |
| chr3 | 26664523 | LRP15 | leucine rich repeat containing 3B | protein-coding |
| chr3 | 126664525 | LRP15 | leucine rich repeat containing 3B | protein-coding |
| chr3 | 111431556 | LL5b\|LL5beta | pleckstrin homology like domain family B | protein-coding |
| | | | member 2 | |
| chr4 | 169799044 | CGI-151\|CGI151\|MYN\|PNCA1\|SIH002 | palladin, cytoskeletal associated protein | protein-coding |
| chr5 | 138441347 | BAP\|MSS\|ULG5 | SIL1 nucleotide exchange factor | protein-coding |
| chr5 | 138441374 | BAP\|MSS\|ULG5 | SIL1 nucleotide exchange factor | protein-coding |
| chr5 | 138441376 | BAP\|MSS\|ULG5 | SIL1 nucleotide exchange factor | protein-coding |
| chr5 | 176038510 | GRIN1 | G protein regulated inducer of neurite | protein-coding |
| | | | outgrowth 1 | |
| chr5 | 176038524 | GRIN1 | G protein regulated inducer of neurite | protein-coding |
| | | | outgrowth 1 | |
| chr5 | 176038525 | GRIN1 | G protein regulated inducer of neurite | protein-coding |
| | | | outgrowth 1 | |
| chr5 | 176038562 | GRIN1 | G protein regulated inducer of neurite | protein-coding |
| | | | outgrowth 1 | |
| chr5 | 179401799 | MIRN340\|hsa-mir-340\|mir-340 | microRNA 340 | ncRNA |
| chr6 | 15265454 | JMJ | jumonji and AT-rich interaction domain | protein-coding |
| | | | containing 2 | |
| chr6 | 168462402 | bA164L23.1 | FERM domain containing 1 | protein-coding |
| chr6 | 168462447 | bA164L23.1 | FERM domain containing 1 | protein-coding |
| chr6 | 170578588 | — | long intergenic non-protein coding RNA | ncRNA |
| | | | 1624 | |
| chr7 | 2968311 | NNX3\|RMP\|gep | G protein subunit alpha 12 | protein-coding |
| chr7 | 2968441 | NNX3\|RMP\|gep | G protein subunit alpha 12 | protein-coding |
| chr7 | 2968442 | NNX3\|RMP\|gep | G protein subunit alpha 12 | protein-coding |
| chr7 | 2968451 | NNX3\|RMP\|gep | G protein subunit alpha 12 | protein-coding |
| chr7 | 2968454 | NNX3\|RMP\|gep | G protein subunit alpha 12 | protein-coding |
| chr7 | 5572006 | BRWS1\|PS1TP5BP1 | actin beta | protein-coding |
| chr7 | 150812220 | AGAP-3\|CENTG3\|CRAG\|MRIP-1\|cnt-g3 | ArfGAP with GTPase domain, ankyrin | protein-coding |
| | | | repeat and PH domain 3 | |
| chr8 | 47570600 | ASNSL1 | asparagine synthetase pseudogene 1 | pseudo |
| chr9 | 116411412 | C2PA\|RGP3 | regulator of G protein signaling 3 | protein-coding |
| chr9 | 124498726 | AF9Q34\|AIP-1\|AIP1\|DIP1/2 | DAB2nteracting protein | protein-coding |
| chr9 | 124620057 | AF9Q34\|AIP-1\|AIP1\|DIP1/2 | DAB2nteracting protein | protein-coding |
| chr10 | 676777 | — | uncharacterized LOC101930421 | ncRNA |
| chr10 | 676811 | — | uncharacterized LOC101930421 | ncRNA |
| chr11 | 2000638 | — | MRPL23 antisense RNA 1 | ncRNA |
| chr11 | 11600192 | GALNACT18\|GALNT15\|GALNTL4\|GalNAc- | polypeptide N- | protein-coding |
| | | T15\|GalNAc-T18 | acetylgalactosaminyltransferase 18 | |
| chr11 | 11600210 | GALNACT18\|GALNT15\|GALNTL4\|GalNAc- | polypeptide N- | protein-coding |
| | | T15\|GalNAc-T18 | acetylgalactosaminyltransferase 18 | |
| chr11 | 11600228 | GALNACT18\|GALNT15\|GALNTL4\|GalNAc- | polypeptide N- | protein-coding |
| | | T15\|GalNAc-T18 | acetylgalactosaminyltransferase 18 | |
| chr11 | 11600230 | GALNACT18\|GALNT15\|GALNTL4\|GalNAc- | polypeptide N- | protein-coding |
| | | T15\|GalNAc-T18 | acetylgalactosaminyltransferase 18 | |
| chr11 | 11600231 | GALNACT18\|GALNT15\|GALNTL4\|GalNAc- | polypeptide N- | protein-coding |
| | | T15\|GalNAc-T18 | acetylgalactosaminyltransferase 18 | |
| chr11 | 11600240 | GALNACT18\|GALNT15\|GALNTL4\|GalNAc- | polypeptide N- | protein-coding |
| | | T15\|GalNAc-T18 | acetylgalactosaminyltransferase 18 | |
| chr11 | 64120601 | MSK2\|RSK-B\|S6K-alpha-4 | ribosomal protein S6 kinase A4 | protein-coding |
| chr11 | 70851727 | AUTS17\|CORTBP1\|CTTNBP1\|ProSAP1\|SHANK\| | SH3 and multiple ankyrin repeat domains 2 | protein-coding |
| | | SPANK-3 | | |
| chr12 | 40618451 | AURA17\|DARDARIN\|PARK8\|RIPK7\|ROCO2 | leucine rich repeat kinase 2 | protein-coding |
| chr12 | 40618493 | AURA17\|DARDARIN\|PARK8\|RIPK7\|ROCO2 | leucine rich repeat kinase 2 | protein-coding |
| chr15 | 32385689 | CHRNA7-2\|NACHRA7 | cholinergic receptor nicotinic alpha 7 | protein-coding |
| | | | subunit | |
| chr15 | 68723747 | HsT18964 | integrin subunit alpha 11 | protein-coding |
| chr15 | 89954601 | — | uncharacterized LOC105371031 | ncRNA |
| chr15 | 89954603 | — | uncharacterized LOC105371031 | ncRNA |
| chr15 | 89954605 | — | uncharacterized LOC105371031 | ncRNA |
| chr15 | 89959942 | — | uncharacterized LOC105371031 | ncRNA |
| chr16 | 85498200 | CRHSP24\|KIAA0182 | Gse1 coiled-coil protein | protein-coding |
| chr16 | 85498219 | CRHSP24\|KIAA0182 | Gse1 coiled-coil protein | protein-coding |
| chr16 | 85498220 | CRHSP24\|KIAA0182 | Gse1 coiled-coil protein | protein-coding |
| chr16 | 85498227 | CRHSP24\|KIAA0182 | Gse1 coiled-coil protein | protein-coding |
| chr16 | 85498781 | CRHSP24\|KIAA0182 | Gse1 coiled-coil protein | protein-coding |
| chr16 | 85519155 | CRHSP24\|KIAA0182 | Gse1 coiled-coil protein | protein-coding |
| chr16 | 85519162 | CRHSP24\|KIAA0182 | Gse1 coiled-coil protein | protein-coding |
| chr16 | 85519165 | CRHSP24\|KIAA0182 | Gse1 coiled-coil protein | protein-coding |
| chr16 | 85519168 | CRHSP24\|KIAA0182 | Gse1 coiled-coil protein | protein-coding |
| chr16 | 85519171 | CRHSP24\|KIAA0182 | Gse1 coiled-coil protein | protein-coding |
| chr16 | 85519173 | CRHSP24\|KIAA0182 | Gse1 coiled-coil protein | protein-coding |
| chr16 | 85519303 | CRHSP24\|KIAA0182 | Gse1 coiled-coil protein | protein-coding |
| chr16 | 88715953 | p22-PHOX | cytochrome b-245 alpha chain | protein-coding |
| chr17 | 28299568 | — | EF-hand calcium binding domain 5 | protein-coding |
| chr17 | 80372745 | HEXDC | hexosaminidase D | protein-coding |
| chr17 | 80372760 | HEXDC | hexosaminidase D | protein-coding |
| chr17 | 80372761 | HEXDC | hexosaminidase D | protein-coding |
| chr17 | 80372766 | HEXDC | hexosaminidase D | protein-coding |
| chr17 | 80372767 | HEXDC | hexosaminidase D | protein-coding |
| chr17 | 80372770 | HEXDC | hexosaminidase D | protein-coding |
| chr17 | 80372771 | HEXDC | hexosaminidase D | protein-coding |
| chr17 | 80372793 | HEXDC | hexosaminidase D | protein-coding |
| chr18 | 57364080 | HKLLS1 | collagen and calcium binding EGF domains | protein-coding |
| | | | 1 | |
| chr19 | 591647 | BCNG-2\|BCNG2\|HAC-1 | hyperpolarization activated cyclic | protein-coding |
| | | | nucleotide gated potassium and sodium | |
| | | | channel 2 | |
| chr19 | 591656 | BCNG-2\|BCNG2\|HAC-1 | hyperpolarization activated cyclic | protein-coding |
| | | | nucleotide gated potassium and sodium | |
| | | | channel 2 | |
| chr19 | 591660 | BCNG-2\|BCNG2\|HAC-1 | hyperpolarization activated cyclic | protein-coding |
| | | | nucleotide gated potassium and sodium | |
| | | | channel 2 | |
| chr19 | 591662 | BCNG-2\|BCNG2\|HAC-1 | hyperpolarization activated cyclic | protein-coding |
| | | | nucleotide gated potassium and sodium | |
| | | | channel 2 | |
| chr19 | 591682 | BCNG-2\|BCNG2\|HAC-1 | hyperpolarization activated cyclic | protein-coding |
| | | | nucleotide gated potassium and sodium | |
| | | | channel 2 | |
| chr19 | 591683 | BCNG-2\|BCNG2\|HAC-1 | hyperpolarization activated cyclic | protein-coding |
| | | | nucleotide gated potassium and sodium | |
| | | | channel 2 | |
| chr19 | 591684 | BCNG-2\|BCNG2\|HAC-1 | hyperpolarization activated cyclic | protein-coding |
| | | | nucleotide gated potassium and sodium | |
| | | | channel 2 | |
| chr19 | 591685 | BCNG-2\|BCNG2\|HAC-1 | hyperpolarization activated cyclic | protein-coding |
| | | | nucleotide gated potassium and sodium | |
| | | | channel 2 | |
| chr19 | 591688 | BCNG-2\|BCNG2\|HAC-1 | hyperpolarization activated cyclic | protein-coding |
| | | | nucleotide gated potassium and sodium | |
| | | | channel 2 | |
| chr19 | 18837089 | MAML2\|MECT1\|Mam-2\|TORC-1\|TORC1\|WAMTP1 | CREB regulated transcription coactivator 1 | protein-coding |
| chr19 | 18837102 | MAML2\|MECT1\|Mam-2\|TORC-1\|TORC1\|WAMTP1 | CREB regulated transcription coactivator 1 | protein-coding |
| chr20 | 3778162 | — | cell division cycle 25B | protein-coding |
| chr20 | 32156432 | EHT\|MTGR1\|ZMYND3\|p85 | CBFA2/RUNX1 partner transcriptional co- | protein-coding |
| | | | repressor 2 | |
| chr20 | 48099327 | DRK1\|Kv2.1 | potassium voltage-gated channel subfamily | protein-coding |
| | | | B member 1 | |
| chr20 | 48099339 | DRK1\|Kv2.1 | potassium voltage-gated channel subfamily | protein-coding |
| | | | B member 1 | |
| chrX | 36981727 | — | family with sequence similarity 47 member | protein-coding |
| | | | C | |
| chrX | 36981729 | — | family with sequence similarity 47 member | protein-coding |
| | | | C | |
| chr1 | 24392969 | — | myomesin 3 | protein-coding |
| chr1 | 24410522 | — | myomesin 3 | protein-coding |
| chr1 | 24434829 | — | myomesin 3 | protein-coding |
| chr1 | 24438565 | — | myomesin 3 | protein-coding |
| chr1 | 27240357 | SHP\|SHP1 | nuclear receptor subfamily 0 group B | protein-coding |
| | | | member 2 | |
| chr1 | 50513586 | HUD\|PNEM | ELAV like RNA binding protein 4 | protein-coding |
| chr1 | 50569482 | HUD\|PNEM | ELAV like RNA binding protein 4 | protein-coding |
| chr1 | 50571262 | HUD\|PNEM | ELAV like RNA binding protein 4 | protein-coding |
| chr1 | 50571864 | HUD\|PNEM | ELAV like RNA binding protein 4 | protein-coding |
| chr1 | 50574505 | HUD\|PNEM | ELAV like RNA binding protein 4 | protein-coding |
| chr1 | 50574538 | HUD\|PNEM | ELAV like RNA binding protein 4 | protein-coding |
| chr1 | 50575198 | HUD\|PNEM | ELAV like RNA binding protein 4 | protein-coding |
| chr1 | 50575278 | HUD\|PNEM | ELAV like RNA binding protein 4 | protein-coding |
| chr1 | 50575388 | HUD\|PNEM | ELAV like RNA binding protein 4 | protein-coding |
| chr1 | 50610525 | HUD\|PNEM | ELAV like RNA binding protein 4 | protein-coding |
| chr1 | 50642702 | HUD\|PNEM | ELAV like RNA binding protein 4 | protein-coding |
| chr1 | 78445126 | FBP\|FUBP\|hDH V | far upstream element binding protein 1 | protein-coding |
| chr1 | 78511486 | SEMCAP-2\|SEMCAP2 | GIPC PDZ domain containing family | protein-coding |
| | | | member 2 | |
| chr1 | 114521963 | HNOEL-iso\|OLF44 | olfactomedin like 3 | protein-coding |
| chr1 | 114521965 | HNOEL-iso\|OLF44 | olfactomedin like 3 | protein-coding |
| chr1 | 114522013 | HNOEL-iso\|OLF44 | olfactomedin like 3 | protein-coding |
| chr1 | 114522595 | HNOEL-iso\|OLF44 | olfactomedin like 3 | protein-coding |
| chr1 | 152956457 | SPRK | small proline rich protein 1A | protein-coding |
| chr1 | 152957607 | SPRK | small proline rich protein 1A | protein-coding |
| chr1 | 153330230 | 60B8AG\|CAGB\|CFAG\|CGLB\|L1AG\|LIAG\| | S100 calcium binding protein A9 | protein-coding |
| | | MAC387\|MIF\|MRP14\|NIF\|P14 | | |
| chr2 | 74119341 | ACT\|ACTA3\|ACTE\|ACTL3\|ACTSG\|VSCM | actin gamma 2, smooth muscle | protein-coding |
| chr2 | 74119978 | ACT\|ACTA3\|ACTE\|ACTL3\|ACTSG\|VSCM | actin gamma 2, smooth muscle | protein-coding |
| chr2 | 74119994 | ACT\|ACTA3\|ACTE\|ACTL3\|ACTSG\|VSCM | actin gamma 2, smooth muscle | protein-coding |
| chr2 | 74120006 | ACT\|ACTA3\|ACTE\|ACTL3\|ACTSG\|VSCM | actin gamma 2, smooth muscle | protein-coding |
| chr2 | 74120009 | ACT\|ACTA3\|ACTE\|ACTL3\|ACTSG\|VSCM | actin gamma 2, smooth muscle | protein-coding |
| chr2 | 74120035 | ACT\|ACTA3\|ACTE\|ACTL3\|ACTSG\|VSCM | actin gamma 2, smooth muscle | protein-coding |
| chr2 | 95940101 | PROML2 | prominin 2 | protein-coding |
| chr2 | 95940121 | PROML2 | prominin 2 | protein-coding |
| chr2 | 95940140 | PROML2 | prominin 2 | protein-coding |
| chr2 | 95940145 | PROML2 | prominin 2 | protein-coding |
| chr2 | 95940149 | PROML2 | prominin 2 | protein-coding |
| chr2 | 95940627 | PROML2 | prominin 2 | protein-coding |
| chr2 | 95943464 | PROML2 | prominin 2 | protein-coding |
| chr2 | 95945514 | PROML2 | prominin 2 | protein-coding |
| chr2 | 95954155 | CSEN\|DREAM\|KCHIP3 | potassium voltage-gated channel interacting | protein-coding |
| | | | protein 3 | |
| chr2 | 95954163 | CSEN\|DREAM\|KCHIP3 | potassium voltage-gated channel interacting | protein-coding |
| | | | protein 3 | |
| chr2 | 169921199 | 3-alpha-HSD\|3ALPHA-HSD\|RDH-E2\|RDH- | dehydrogenase/reductase 9 | protein-coding |
| | | TBE\|RDH15\|RDHL\|RDHTBE\|RETSDR8\|SDR9C4 | | |
| chr2 | 169923445 | 3-alpha-HSD\|3ALPHA-HSD\|RDH-E2\|RDH- | dehydrogenase/reductase 9 | protein-coding |
| | | TBE\|RDH15\|RDHL\|RDHTBE\|RETSDR8\|SDR9C4 | | |
| chr2 | 169923487 | 3-alpha-HSD\|3ALPHA-HSD\|RDH-E2\|RDH- | dehydrogenase/reductase 9 | protein-coding |
| | | TBE\|RDH15\|RDHL\|RDHTBE\|RETSDR8\|SDR9C4 | | |
| chr2 | 169925966 | 3-alpha-HSD\|3ALPHA-HSD\|RDH-E2\|RDH- | dehydrogenase/reductase 9 | protein-coding |
| | | TBE\|RDH15\|RDHL\|RDHTBE\|RETSDR8\|SDR9C4 | | |
| chr2 | 169925972 | 3-alpha-HSD\|3ALPHA-HSD\|RDH-E2\|RDH- | dehydrogenase/reductase 9 | protein-coding |
| | | TBE\|RDH15\|RDHL\|RDHTBE\|RETSDR8\|SDR9C4 | | |
| chr2 | 169925986 | 3-alpha-HSD\|3ALPHA-HSD\|RDH-E2\|RDH- | dehydrogenase/reductase 9 | protein-coding |
| | | TBE\|RDH15\|RDHL\|RDHTBE\|RETSDR8\|SDR9C4 | | |
| chr2 | 169928910 | 3-alpha-HSD\|3ALPHA-HSD\|RDH-E2\|RDH- | dehydrogenase/reductase 9 | protein-coding |
| | | TBE\|RDH15\|RDHL\|RDHTBE\|RETSDR8\|SDR9C4 | | |
| chr2 | 169937517 | 3-alpha-HSD\|3ALPHA-HSD\|RDH-E2\|RDH- | dehydrogenase/reductase 9 | protein-coding |
| | | TBE\|RDH15\|RDHL\|RDHTBE\|RETSDR8\|SDR9C4 | | |
| chr2 | 177001240 | — | uncharacterized LOC401021 | ncRNA |
| chr2 | 177001585 | — | HOXD cluster antisense RNA 2 | ncRNA |
| chr2 | 177015022 | MIRN10B\|hsa-mir-10b\|miRNA10B\|mir-10b | microRNA 10b | ncRNA |
| chr2 | 177025519 | HOX1D\|HOX4\|HOX4A\|Hox-4.1 | homeobox D3 | protein-coding |
| chr2 | 177028705 | HOX1D\|HOX4\|HOX4A\|Hox-4.1 | homeobox D3 | protein-coding |
| chr2 | 182545174 | BETA2\|BHF-1\|MODY6\|NEUROD\|bHLHa3 | neuronal differentiation 1 | protein-coding |
| chr2 | 182545503 | BETA2\|BHF-1\|MODY6\|NEUROD\|bHLHa3 | neuronal differentiation 1 | protein-coding |
| chr2 | 219696414 | AMPKG3 | protein kinase AMP-activated non-catalytic | protein-coding |
| | | | subunit gamma 3 | |
| chr2 | 219696424 | AMPKG3 | protein kinase AMP-activated non-catalytic | protein-coding |
| | | | subunit gamma 3 | |
| chr2 | 219696426 | AMPKG3 | protein kinase AMP-activated non-catalytic | protein-coding |
| | | | subunit gamma 3 | |
| chr2 | 219696439 | AMPKG3 | protein kinase AMP -activated non-catalytic | protein-coding |
| | | | subunit gamma 3 | |
| chr2 | 219696709 | AMPKG3 | protein kinase AMP -activated non-catalytic | protein-coding |
| | | | subunit gamma 3 | |
| chr2 | 223916432 | MIRP3 | potassium voltage-gated channel subfamily | protein-coding |
| | | | E regulatory subunit 4 | |
| chr2 | 223916762 | MIRP3 | potassium voltage-gated channel subfamily | protein-coding |
| | | | E regulatory subunit 4 | |
| chr2 | 223916818 | MIRP3 | potassium voltage-gated channel subfamily | protein-coding |
| | | | E regulatory subunit 4 | |
| chr2 | 233924577 | SHIP\|SHIP-1\|SHIP1\|SIP-145\|hp51CN\|p150Ship | inositol polyphosphate-5-phosphatase D | protein-coding |
| chr2 | 233924962 | SHIP\|SHIP-1\|SHIP1\|SIP-145\|hp51CN\|p150Ship | inositol polyphosphate-5-phosphatase D | protein-coding |
| chr2 | 233925089 | SHIP\|SHIP-1\|SHIP1\|SIP-145\|hp51CN\|p150Ship | inositol polyphosphate-5-phosphatase D | protein-coding |
| chr2 | 233940872 | SHIP\|SHIP-1\|SHIP1\|SIP-145\|hp51CN\|p150Ship | inositol polyphosphate-5-phosphatase D | protein-coding |
| chr2 | 233985212 | SHIP\|SHIP-1\|SHIP1\|SIP-145\|hp51CN\|p150Ship | inositol polyphosphate-5-phosphatase D | protein-coding |
| chr2 | 233986826 | SHIP\|SHIP-1\|SHIP1\|SIP-145\|hp51CN\|p150Ship | inositol polyphosphate-5-phosphatase D | protein-coding |
| chr2 | 234053819 | APG16L\|ATG16A\|ATG16L\|IBD10\|WDR30 | autophagy related 16 like 1 | protein-coding |
| chr2 | 234054695 | APG16L\|ATG16A\|ATG16L\|IBD10\|WDR30 | autophagy related 16 like 1 | protein-coding |
| chr2 | 234070270 | APG16L\|ATG16A\|ATG16L\|IBD10\|WDR30 | autophagy related 16 like 1 | protein-coding |
| chr2 | 234070335 | APG16L\|ATG16A\|ATG16L\|IBD10\|WDR30 | autophagy related 16 like 1 | protein-coding |
| chr2 | 234072847 | APG16L\|ATG16A\|ATG16L\|IBD10\|WDR30 | autophagy related 16 like 1 | protein-coding |
| chr2 | 234078106 | APG16L\|ATG16A\|ATG16L\|IBD10\|WDR30 | autophagy related 16 like 1 | protein-coding |
| chr2 | 234078567 | APG16L\|ATG16A\|ATG16L\|IBD10\|WDR30 | autophagy related 16 like 1 | protein-coding |
| chr2 | 234078920 | APG16L\|ATG16A\|ATG16L\|IBD10\|WDR30 | autophagy related 16 like 1 | protein-coding |
| chr2 | 234103983 | APG16L\|ATG16A\|ATG16L\|IBD10\|WDR30 | autophagy related 16 like 1 | protein-coding |
| chr2 | 234113188 | APG16L\|ATG16A\|ATG16L\|IBD10\|WDR30 | autophagy related 16 like 1 | protein-coding |
| chr3 | 12837871 | TIP120B\|Tp120b | cullin associated and neddylation | protein-coding |
| | | | dissociated 2 (putative) | |
| chr3 | 12838071 | TIP120B\|Tp120b | cullin associated and neddylation | protein-coding |
| | | | dissociated 2 (putative) | |
| chr3 | 12838079 | TIP120B\|Tp120b | cullin associated and neddylation | protein-coding |
| | | | dissociated 2 (putative) | |
| chr3 | 12848532 | TIP120B\|Tp120b | cullin associated and neddylation | protein-coding |
| | | | dissociated 2 (putative) | |
| chr3 | 12868845 | L32\|PP9932 | ribosomal protein L32 | protein-coding |
| chr3 | 42726911 | KBTBD5\|NEM8\|SRYP\|SYRP | kelch like family member 40 | protein-coding |
| chr3 | 111717411 | NP22\|NP24\|NP25 | transgelin 3 | protein-coding |
| chr3 | 111717486 | NP22\|NP24\|NP25 | transgelin 3 | protein-coding |
| chr3 | 111717542 | NP22\|NP24\|NP25 | transgelin 3 | protein-coding |
| chr3 | 111717936 | NP22\|NP24\|NP25 | transgelin 3 | protein-coding |
| chr3 | 111718095 | NP22\|NP24\|NP25 | transgelin 3 | protein-coding |
| chr3 | 111719270 | NP22\|NP24\|NP25 | transgelin 3 | protein-coding |
| chr3 | 111719519 | NP22\|NP24\|NP25 | transgelin 3 | protein-coding |
| chr3 | 128200821 | DCML\|IMD21\|MONOMAC\|NFE1B | GATA binding protein 2 | protein-coding |
| chr3 | 128206659 | DCML\|IMD21\|MONOMAC\|NFE1B | GATA binding protein 2 | protein-coding |
| chr3 | 128207273 | DCML\|IMD21\|MONOMAC\|NFE1B | GATA binding protein 2 | protein-coding |
| chr3 | 128209391 | — | GATA2 antisense RNA 1 | ncRNA |
| chr3 | 128211916 | DCML\|IMD21\|MONOMAC\|NFE1B | GATA binding protein 2 | protein-coding |
| chr3 | 128211928 | DCML\|IMD21\|MONOMAC\|NFE1B | GATA binding protein 2 | protein-coding |
| chr3 | 150481064 | hSiah2 | siah E3 ubiquitin protein ligase 2 | protein-coding |
| chr3 | 150481118 | hSiah2 | siah E3 ubiquitin protein ligase 2 | protein-coding |
| chr3 | 150481164 | hSiah2 | siah E3 ubiquitin protein ligase 2 | protein-coding |
| chr3 | 151154610 | CMF608 | immunoglobulin superfamily member 10 | protein-coding |
| chr3 | 151158459 | CMF608 | immunoglobulin superfamily member 10 | protein-coding |
| chr3 | 151158468 | CMF608 | immunoglobulin superfamily member 10 | protein-coding |
| chr3 | 151160838 | CMF608 | immunoglobulin superfamily member 10 | protein-coding |
| chr3 | 151176397 | CMF608 | immunoglobulin superfamily member 10 | protein-coding |
| chr3 | 181429614 | ANOP3\|MCOPS3 | SRY-box transcription factor 2 | protein-coding |
| chr3 | 181429622 | ANOP3\|MCOPS3 | SRY-box transcription factor 2 | protein-coding |
| chr3 | 185782249 | ERM | ETS variant 5 | protein-coding |
| chr3 | 185783659 | ERM | ETS variant 5 | protein-coding |
| chr3 | 185796794 | ERM | ETS variant 5 | protein-coding |
| chr3 | 185797595 | ERM | ETS variant 5 | protein-coding |
| chr3 | 185823179 | ERM | ETS variant 5 | protein-coding |
| chr3 | 185823413 | ERM | ETS variant 5 | protein-coding |
| chr3 | 185823631 | ERM | ETS variant 5 | protein-coding |
| chr3 | 185826233 | ERM | ETS variant 5 | protein-coding |
| chr3 | 185826239 | ERM | ETS variant 5 | protein-coding |
| chr3 | 185826364 | ERM | ETS variant 5 | protein-coding |
| chr3 | 185826618 | ERM | ETS variant 5 | protein-coding |
| chr3 | 185826688 | ERM | ETS variant 5 | protein-coding |
| chr3 | 185826770 | ERM | ETS variant 5 | protein-coding |
| chr3 | 185826801 | ERM | ETS variant 5 | protein-coding |
| chr3 | 185828007 | ERM | ETS variant 5 | protein-coding |
| chr3 | 187086020 | IFRG28\|Z3CXXC4 | receptor transporter protein 4 | protein-coding |
| chr4 | 6694696 | M1G9 | S100 calcium binding protein P | protein-coding |
| chr4 | 6695505 | M1G9 | S100 calcium binding protein P | protein-coding |
| chr4 | 38796154 | CD281\|TIL\|TIL. LPRS5\|rsc786 | toll like receptor 1 | protein-coding |
| chr4 | 38805897 | CD281\|TIL\|TIL. LPRS5\|rsc786 | toll like receptor 1 | protein-coding |
| chr4 | 38806310 | CD281\|TIL\|TIL. LPRS5\|rsc786 | toll like receptor 1 | protein-coding |
| chr4 | 38806312 | CD281\|TIL\|TIL. LPRS5\|rsc786 | toll like receptor 1 | protein-coding |
| chr4 | 38806340 | CD281\|TIL\|TIL. LPRS5\|rsc786 | toll like receptor 1 | protein-coding |
| chr4 | 38806372 | CD281\|TIL\|TIL. LPRS5\|rsc786 | toll like receptor 1 | protein-coding |
| chr4 | 38806437 | CD281\|TIL\|TIL. LPRS5\|rsc786 | toll like receptor 1 | protein-coding |
| chr4 | 38807054 | CD281\|TIL\|TIL. LPRS5\|rsc786 | toll like receptor 1 | protein-coding |
| chr4 | 38807099 | CD281\|TIL\|TIL. LPRS5\|rsc786 | toll like receptor 1 | protein-coding |
| chr4 | 38858338 | CD286 | toll like receptor 6 | protein-coding |
| chr4 | 41258330 | — | UCHL1 antisense RNA 1 (head to head) | ncRNA |
| chr4 | 41258686 | — | UCHL1 antisense RNA 1 (head to head) | ncRNA |
| chr4 | 41258750 | HEL-117\|HEL-S-53\|NDGOA\|PARK5\|PGP9.5\| | ubiquitin C-terminal hydrolase L1 | protein-coding |
| | | PGP9.5\|PGP95\|SPG79\|Uch-L1 | | |
| chr4 | 41258825 | HEL-117\|HEL-S-53\|NDGOA\|PARK5\|PGP9.5\| | ubiquitin C-terminal hydrolase L1 | protein-coding |
| | | PGP9.5\|PGP95\|SPG79\|Uch-L1 | | |
| chr4 | 41258828 | HEL-117\|HEL-S-53\|NDGOA\|PARK5\|PGP9.5\| | ubiquitin C-terminal hydrolase L1 | protein-coding |
| | | PGP9.5\|PGP95\|SPG79\|Uch-L1 | | |
| chr4 | 41258849 | HEL-117\|HEL-S-53\|NDGOA\|PARK5\|PGP9.5\| | ubiquitin C-terminal hydrolase L1 | protein-coding |
| | | PGP9.5\|PGP95\|SPG79\|Uch-L1 | | |
| chr4 | 41258850 | HEL-117\|HEL-S-53\|NDGOA\|PARK5\|PGP9.5\| | ubiquitin C-terminal hydrolase L1 | protein-coding |
| | | PGP9.5\|PGP95\|SPG79\|Uch-L1 | | |
| chr4 | 41258857 | HEL-117\|HEL-S-53\|NDGOA\|PARK5\|PGP9.5\| | ubiquitin C-terminal hydrolase L1 | protein-coding |
| | | PGP9.5\|PGP95\|SPG79\|Uch-L1 | | |
| chr4 | 41258862 | HEL-117\|HEL-S-53\|NDGOA\|PARK5\|PGP9.5\| | ubiquitin C-terminal hydrolase L1 | protein-coding |
| | | PGP9.5\|PGP95\|SPG79\|Uch-L1 | | |
| chr4 | 41258863 | HEL-117\|HEL-S-53\|NDGOA\|PARK5\|PGP9.5\| | ubiquitin C-terminal hydrolase L1 | protein-coding |
| | | PGP9.5\|PGP95\|SPG79\|Uch-L1 | | |
| chr4 | 41262556 | HEL-117\|HEL-S-53\|NDGOA\|PARK5\|PGP9.5\| | ubiquitin C-terminal hydrolase L1 | protein-coding |
| | | PGP9.5\|PGP95\|SPG79\|Uch-L1 | | |
| chr4 | 41263656 | HEL-117\|HEL-S-53\|NDGOA\|PARK5\|PGP9.5\| | ubiquitin C-terminal hydrolase L1 | protein-coding |
| | | PGP9.5\|PGP95\|SPG79\|Uch-L1 | | |
| chr4 | 48899120 | — | OCIA domain containing 2 | protein-coding |
| chr4 | 48899765 | — | OCIA domain containing 2 | protein-coding |
| chr4 | 48908644 | — | OCIA domain containing 2 | protein-coding |
| chr4 | 48908674 | — | OCIA domain containing 2 | protein-coding |
| chr4 | 48908691 | — | OCIA domain containing 2 | protein-coding |
| chr4 | 48908705 | — | OCIA domain containing 2 | protein-coding |
| chr4 | 48908715 | — | OCIA domain containing 2 | protein-coding |
| chr4 | 48908745 | — | OCIA domain containing 2 | protein-coding |
| chr4 | 48908854 | — | OCIA domain containing 2 | protein-coding |
| chr4 | 84030896 | C15\|DGIC\|PNAS-144\|onzin | placenta associated 8 | protein-coding |
| chr4 | 84035768 | C15\|DGIC\|PNAS-144\|onzin | placenta associated 8 | protein-coding |
| chr4 | 84035789 | C15\|DGIC\|PNAS-144\|onzin | placenta associated 8 | protein-coding |
| chr4 | 84035855 | C15\|DGIC\|PNAS-144\|onzin | placenta associated 8 | protein-coding |
| chr4 | 84058128 | C15\|DGIC\|PNAS-144\|onzin | placenta associated 8 | protein-coding |
| chr4 | 177158625 | ASB-5 | ankyrin repeat and SOCS box containing 5 | protein-coding |
| chr4 | 177162727 | ASB-5 | ankyrin repeat and SOCS box containing 5 | protein-coding |
| chr4 | 177170609 | ASB-5 | ankyrin repeat and SOCS box containing 5 | protein-coding |
| chr4 | 177190273 | ASB-5 | ankyrin repeat and SOCS box containing 5 | protein-coding |
| chr4 | 177198622 | ASB-5 | ankyrin repeat and SOCS box containing 5 | protein-coding |
| chr5 | 102089585 | PAL\|PHM | peptidylglycine alpha-amidating | protein-coding |
| | | | monooxygenase | |
| chr5 | 102090271 | PAL\|PHM | peptidylglycine alpha-amidating | protein-coding |
| | | | monooxygenase | |
| chr5 | 102090387 | PAL\|PHM | peptidylglycine alpha-amidating | protein-coding |
| | | | monooxygenase | |
| chr5 | 102090918 | PAL\|PHM | peptidylglycine alpha-amidating | protein-coding |
| | | | monooxygenase | |
| chr5 | 102090947 | PAL\|PHM | peptidylglycine alpha-amidating | protein-coding |
| | | | monooxygenase | |
| chr5 | 102143661 | PAL\|PHM | peptidylglycine alpha-amidating | protein-coding |
| | | | monooxygenase | |
| chr5 | 102143667 | PAL\|PHM | peptidylglycine alpha-amidating | protein-coding |
| | | | monooxygenase | |
| chr5 | 102200141 | PAL\|PHM | peptidylglycine alpha-amidating | protein-coding |
| | | | monooxygenase | |
| chr5 | 102200848 | PAL\|PHM | peptidylglycine alpha-amidating | protein-coding |
| | | | monooxygenase | |
| chr5 | 102200914 | PAL\|PHM | peptidylglycine alpha-amidating | protein-coding |
| | | | monooxygenase | |
| chr5 | 102201083 | PAL\|PHM | peptidylglycine alpha-amidating | protein-coding |
| | | | monooxygenase | |
| chr5 | 102201330 | PAL\|PHM | peptidylglycine alpha-amidating | protein-coding |
| | | | monooxygenase | |
| chr5 | 102201341 | PAL\|PHM | peptidylglycine alpha-amidating | protein-coding |
| | | | monooxygenase | |
| chr5 | 102201423 | PAL\|PHM | peptidylglycine alpha-amidating | protein-coding |
| | | | monooxygenase | |
| chr5 | 102201427 | PAL\|PHM | peptidylglycine alpha-amidating | protein-coding |
| | | | monooxygenase | |
| chr5 | 102201614 | PAL\|PHM | peptidylglycine alpha-amidating | protein-coding |
| | | | monooxygenase | |
| chr5 | 102201661 | PAL\|PHM | peptidylglycine alpha-amidating | protein-coding |
| | | | monooxygenase | |
| chr5 | 102201720 | PAL\|PHM | peptidylglycine alpha-amidating | protein-coding |
| | | | monooxygenase | |
| chr5 | 102202191 | PAL\|PHM | peptidylglycine alpha-amidating | protein-coding |
| | | | monooxygenase | |
| chr5 | 102231517 | PAL\|PHM | peptidylglycine alpha-amidating | protein-coding |
| | | | monooxygenase | |
| chr5 | 102285181 | PAL\|PHM | peptidylglycine alpha-amidating | protein-coding |
| | | | monooxygenase | |
| chr5 | 102295601 | PAL\|PHM | peptidylglycine alpha-amidating | protein-coding |
| | | | monooxygenase | |
| chr5 | 102325375 | PAL\|PHM | peptidylglycine alpha-amidating | protein-coding |
| | | | monooxygenase | |
| chr5 | 102343057 | GIN-1\|TGIN1\|ZH2C2 | gypsy retrotransposon integrase 1 | protein-coding |
| chr5 | 102343146 | GIN-1\|TGIN1\|ZH2C2 | gypsy retrotransposon integrase 1 | protein-coding |
| chr5 | 102343163 | GIN-1\|TGIN1\|ZH2C2 | gypsy retrotransposon integrase 1 | protein-coding |
| chr5 | 102343173 | GIN-1\|TGIN1\|ZH2C2 | gypsy retrotransposon integrase 1 | protein-coding |
| chr6 | 34523991 | PDEF\|bA375E1.3 | SAM pointed domain containing ETS | protein-coding |
| | | | transcription factor | |
| chr6 | 34524010 | PDEF\|bA375E1.3 | SAM pointed domain containing ETS | protein-coding |
| | | | transcription factor | |
| chr6 | 112451090 | C6orf225 | family with sequence similarity 229 | protein-coding |
| | | | member B | |
| chr6 | 112451261 | C6orf225 | family with sequence similarity 229 | protein-coding |
| | | | member B | |
| chr6 | 112460378 | C6orf225 | family with sequence similarity 229 | protein-coding |
| | | | member B | |
| chr6 | 112486266 | — | uncharacterized LOC10192764U | ncRNA |
| chr6 | 112500604 | — | uncharacterized LOC10192764U | ncRNA |
| chr6 | 112512866 | — | uncharacterized LOC10192764U | ncRNA |
| chr6 | 112512903 | — | uncharacterized LOC101927640 | ncRNA |
| chr6 | 112512913 | — | uncharacterized LOC101927640 | ncRNA |
| chr6 | 112537550 | — | uncharacterized LOC101927640 | ncRNA |
| chr6 | 112537855 | — | uncharacterized LOC101927640 | ncRNA |
| chr6 | 112575587 | CMD1JJ\|LAMA3\|LAMA4*-1 | laminin subunit alpha 4 | protein-coding |
| chr6 | 112575640 | CMD1JJ\|LAMA3\|LAMA4*-1 | laminin subunit alpha 4 | protein-coding |
| chr6 | 112575674 | CMD1JJ\|LAMA3\|LAMA4*-1 | laminin subunit alpha 4 | protein-coding |
| chr6 | 112575680 | CMD1JJ\|LAMA3\|LAMA4*-1 | laminin subunit alpha 4 | protein-coding |
| chr6 | 112575690 | CMD1JJ\|LAMA3\|LAMA4*-1 | laminin subunit alpha 4 | protein-coding |
| chr6 | 112575702 | CMD1JJ\|LAMA3\|LAMA4*-1 | laminin subunit alpha 4 | protein-coding |
| chr6 | 112575749 | CMD1JJ\|LAMA3\|LAMA4*-1 | laminin subunit alpha 4 | protein-coding |
| chr6 | 112575812 | CMD1JJ\|LAMA3\|LAMA4*-1 | laminin subunit alpha 4 | protein-coding |
| chr6 | 112575817 | CMD1JJ\|LAMA3\|LAMA4*-1 | laminin subunit alpha 4 | protein-coding |
| chr6 | 112575824 | CMD1JJ\|LAMA3\|LAMA4*-1 | laminin subunit alpha 4 | protein-coding |
| chr6 | 112576041 | CMD1JJ\|LAMA3\|LAMA4*-1 | laminin subunit alpha 4 | protein-coding |
| chr7 | 148508815 | ENX-1\|ENX1\|EZH2b\|KMT6\|KMT6A\|WVS\|WVS2 | enhancer of zeste 2 polycomb repressive | protein-coding |
| | | | complex 2 subunit | |
| chr7 | 148580501 | ENX-1\|ENX1\|EZH2b\|KMT6\|KMT6A\|WVS\|WVS2 | enhancer of zeste 2 polycomb repressive | protein-coding |
| | | | complex 2 subunit | |
| chr7 | 148581151 | ENX-1\|ENX1\|EZH2b\|KMT6\|KMT6A\|WVS\|WVS2 | enhancer of zeste 2 polycomb repressive | protein-coding |
| | | | complex 2 subunit | |
| chr7 | 148581237 | ENX-1\|ENX1\|EZH2b\|KMT6\|KMT6A\|WVS\|WVS2 | enhancer of zeste 2 polycomb repressive | protein-coding |
| | | | complex 2 subunit | |
| chr7 | 148581240 | ENX-1\|ENX1\|EZH2b\|KMT6\|KMT6A\|WVS\|WVS2 | enhancer of zeste 2 polycomb repressive | protein-coding |
| | | | complex 2 subunit | |
| chr7 | 148581270 | ENX-1\|ENX1\|EZH2b\|KMT6\|KMT6A\|WVS\|WVS2 | enhancer of zeste 2 polycomb repressive | protein-coding |
| | | | complex 2 subunit | |
| chr7 | 148581275 | ENX-1\|ENX1\|EZH2b\|KMT6\|KMT6A\|WVS\|WVS2 | enhancer of zeste 2 polycomb repressive | protein-coding |
| | | | complex 2 subunit | |
| chr7 | 148581279 | ENX-1\|ENX1\|EZH2b\|KMT6\|KMT6A\|WVS\|WVS2 | enhancer of zeste 2 polycomb repressive | protein-coding |
| | | | complex 2 subunit | |
| chr7 | 148581313 | ENX-1\|ENX1\|EZH2b\|KMT6\|KMT6A\|WVS\|WVS2 | enhancer of zeste 2 polycomb repressive | protein-coding |
| | | | complex 2 subunit | |
| chr8 | 17169082 | HCRP1\|PQBP2\|SPG53 | VPS37A subunit of ESCRT-1 | protein-coding |
| chr8 | 17169272 | HCRP1\|PQBP2\|SPG53 | VPS37A subunit of ESCRT-1 | protein-coding |
| chr8 | 17206466 | — | myotubularin related protein 7 | protein-coding |
| chr8 | 17206646 | — | myotubularin related protein 7 | protein-coding |
| chr8 | 17206894 | — | myotubularin related protein 7 | protein-coding |
| chr8 | 17270716 | — | myotubularin related protein 7 | protein-coding |
| chr8 | 17270736 | — | myotubularin related protein 7 | protein-coding |
| chr8 | 17270937 | — | myotubularin related protein 7 | protein-coding |
| chr8 | 53045604 | — | uncharacterized LOC101929341 | ncRNA |
| chr8 | 53045678 | — | uncharacterized LOC101929341 | ncRNA |
| chr8 | 53050272 | — | uncharacterized LOC101929341 | ncRNA |
| chr8 | 53130239 | — | uncharacterized LOC101929341 | ncRNA |
| chr8 | 5313028 | — | uncharacterized LOC101929341 | ncRNA |
| chr8 | 53130282 | — | uncharacterized LOC101929341 | ncRNA |
| chr8 | 53134555 | NZF-3\|NZF3\|ZC2H2C3\|ZC2HC10\|ZNF387 | ST18 C2H2C-type zinc finger transcription | protein-coding |
| | | | factor | |
| chr8 | 53159124 | NZF-3\|NZF3\|ZC2H2C3\|ZC2HC10\|ZNF387 | ST18 C2H2C-type zinc finger transcription | protein-coding |
| | | | factor | |
| chr8 | 53160986 | NZF-3\|NZF3\|ZC2H2C3\|ZC2HC10\|ZNF387 | ST18 C2H2C-type zinc finger transcription | protein-coding |
| | | | factor | |
| chr8 | 53161872 | NZF-3\|NZF3\|ZC2H2C3\|ZC2HC10\|ZNF387 | ST18 C2H2C-type zinc finger transcription | protein-coding |
| | | | factor | |
| chr8 | 53166808 | NZF-3\|NZF3\|ZC2H2C3\|ZC2HC10\|ZNF387 | ST18 C2H2C-type zinc finger transcription | protein-coding |
| | | | factor | |
| chr8 | 53322203 | NZF-3\|NZF3\|ZC2H2C3\|ZC2HC10\|ZNF387 | ST18 C2H2C-type zinc finger transcription | protein-coding |
| | | | factor | |
| chr8 | 53322209 | NZF-3\|NZF3\|ZC2H2C3\|ZC2HC10\|ZNF387 | ST18 C2H2C-type zinc finger transcription | protein-coding |
| | | | factor | |
| chr8 | 53322216 | NZF-3\|NZF3\|ZC2H2C3\|ZC2HC10\|ZNF387 | ST18 C2H2C-type zinc finger transcription | protein-coding |
| | | | factor | |
| chr8 | 53322232 | NZF-3\|NZF3\|ZC2H2C3\|ZC2HC10\|ZNF387 | ST18 C2H2C-type zinc finger transcription | protein-coding |
| | | | factor | |
| chr8 | 53322252 | NZF-3\|NZF3\|ZC2H2C3\|ZC2HC10\|ZNF387 | ST18 C2H2C-type zinc finger transcription | protein-coding |
| | | | factor | |
| chr8 | 53322265 | NZF-3\|NZF3\|ZC2H2C3\|ZC2HC10\|ZNF387 | ST18 C2H2C-type zinc finger transcription | protein-coding |
| | | | factor | |
| chr8 | 53322339 | NZF-3\|NZF3\|ZC2H2C3\|ZC2HC10\|ZNF387 | ST18 C2H2C-type zinc finger transcription | protein-coding |
| | | | factor | |
| chr8 | 53322342 | NZF-3\|NZF3\|ZC2H2C3\|ZC2HC10\|ZNF387 | ST18 C2H2C-type zinc finger transcription | protein-coding |
| | | | factor | |
| chr8 | 53322405 | NZF-3\|NZF3\|ZC2H2C3\|ZC2HC10\|ZNF387 | ST18 C2H2C-type zinc finger transcription | protein-coding |
| | | | factor | |
| chr8 | 53373419 | NZF-3\|NZF3\|ZC2H2C3\|ZC2HC10\|ZNF387 | ST18 C2H2C-type zinc finger transcription | protein-coding |
| | | | factor | |
| chr8 | 104152838 | — | BAALC binder of MAP3K1 and KLF4 | protein-coding |
| chr8 | 104152841 | — | BAALC binder of MAP3K1 and KLF4 | protein-coding |
| chr8 | 104152887 | — | BAALC binder of MAP3K1 and KLF4 | protein-coding |
| chr8 | 104153026 | — | BAALC binder of MAP3K1 and KLF4 | protein-coding |
| chr8 | 104165267 | mir-3151 | microRNA 3151 | ncRNA |
| chr8 | 104220320 | FZD6-DT\|lncFZD6 | BAALC antisense RNA 1 | ncRNA |
| chr8 | 104225459 | FZD6-DT\|lncFZD6 | BAALC antisense RNA 1 | ncRNA |
| chr8 | 104225539 | FZD6-DT\|lncFZD6 | BAALC antisense RNA 1 | ncRNA |
| chr10 | 69597717 | HPANBH4\|JDP1 | DnaJ heat shock protein family (Hsp40) | protein-coding |
| | | | member C12 | |
| chr10 | 69597721 | HPANBH4\|JDP1 | DnaJ heat shock protein family (Hsp40) | protein-coding |
| | | | member C12 | |
| chr10 | 69597754 | HPANBH4\|JDP1 | DnaJ heat shock protein family (Hsp40) | protein-coding |
| | | | member C12 | |
| chr10 | 69597822 | HPANBH4\|JDP1 | DnaJ heat shock protein family (Hsp40) | protein-coding |
| | | | member C12 | |
| chr10 | 69597824 | HPANBH4\|JDP1 | DnaJ heat shock protein family (Hsp40) | protein-coding |
| | | | member C12 | |
| chr10 | 88428106 | CMD1C\|CMH24\|CMPD3\|CYPHER\|LDB3Z1\|LDB3Z4\| | LIM domain binding 3 | protein-coding |
| | | LVNC3\|MFM4\|ORACLE\|PDLIM6\|ZASP | | |
| chr10 | 88428270 | CMD1C\|CMH24\|CMPD3\|CYPHER\|LDB3Z1\|LDB3Z4\| | LIM domain binding 3 | protein-coding |
| | | LVNC3\|MFM4\|ORACLE\|PDLIM6\|ZASP | | |
| chr10 | 88428326 | CMD1C\|CMH24\|CMPD3\|CYPHER\|LDB3Z1\|LDB3Z4\| | LIM domain binding 3 | protein-coding |
| | | LVNC3\|MFM4\|ORACLE\|PDLIM6\|ZASP | | |
| chr10 | 88428328 | CMD1C\|CMH24\|CMPD3\|CYPHER\|LDB3Z1\|LDB3Z4\| | LIM domain binding 3 | protein-coding |
| | | LVNC3\|MFM4\|ORACLE\|PDLIM6\|ZASP | | |
| chr10 | 88428349 | CMD1C\|CMH24\|CMPD3\|CYPHER\|LDB3Z1\|LDB3Z4\| | LIM domain binding 3 | protein-coding |
| | | LVNC3\|MFM4\|ORACLE\|PDLIM6\|ZASP | | |
| chr10 | 88478387 | 10q23del\|ACVRLK3\|ALK3\|CD292\|SKR5 | bone morphogenetic protein receptor type | protein-coding |
| | | | 1A | |
| chr10 | 92680933 | ALRP\|C-193\|CARP\|CVARP\|MCARP\|bA320F15.2 | ankyrin repeat domain 1 | protein-coding |
| chr11 | 60869767 | LEU1\|T1 | CD5 molecule | protein-coding |
| chr11 | 60869881 | LEU1\|T1 | CD5 molecule | protein-coding |
| chr11 | 67350966 | DFN7\|FAEES3\|GST3\|GSTP\|HEL-S-22\|PI | glutathione S-transferase pi 1 | protein-coding |
| chr11 | 67351113 | DFN7\|FAEES3\|GST3\|GSTP\|HEL-S-22\|PI | glutathione S-transferase pi 1 | protein-coding |
| chr11 | 67351193 | DFN7\|FAEES3\|GST3\|GSTP\|HEL-S-22\|PI | glutathione S-transferase pi 1 | protein-coding |
| chr11 | 67351495 | DFN7\|FAEES3\|GST3\|GSTP\|HEL-S-22\|PI | glutathione S-transferase pi 1 | protein-coding |
| chr11 | 67351496 | DFN7\|FAEES3\|GST3\|GSTP\|HEL-S-22\|PI | glutathione S-transferase pi 1 | protein-coding |
| chr11 | 67351505 | DFN7\|FAEES3\|GST3\|GSTP\|HEL-S-22\|PI | glutathione S-transferase pi 1 | protein-coding |
| chr11 | 67351826 | DFN7\|FAEES3\|GST3\|GSTP\|HEL-S-22\|PI | glutathione S-transferase pi 1 | protein-coding |
| chr11 | 67352166 | DFN7\|FAEES3\|GST3\|GSTP\|HEL-S-22\|PI | glutathione S-transferase pi 1 | protein-coding |
| chr11 | 67353475 | DFN7\|FAEES3\|GST3\|GSTP\|HEL-S-22\|PI | glutathione S-transferase pi 1 | protein-coding |
| chr11 | 87908501 | NY-MEL-1\|rrGTPbp | RAB38, member RAS oncogene family | protein-coding |
| chr11 | 87908502 | NY-MEL-1\|rrGTPbp | RAB38, member RAS oncogene family | protein-coding |
| chr11 | 87908535 | NY-MEL-1\|rrGTPbp | RAB38, member RAS oncogene family | protein-coding |
| chr11 | 112831897 | CD56\|MSK39\|NCAM | neural cell adhesion molecule 1 | protein-coding |
| chr11 | 112831990 | CD56\|MSK39\|NCAM | neural cell adhesion molecule 1 | protein-coding |
| chr11 | 112832024 | CD56\|MSK39\|NCAM | neural cell adhesion molecule 1 | protein-coding |
| chr11 | 112832047 | CD56\|MSK39\|NCAM | neural cell adhesion molecule 1 | protein-coding |
| chr11 | 112832057 | CD56\|MSK39\|NCAM | neural cell adhesion molecule 1 | protein-coding |
| chr11 | 112832058 | CD56\|MSK39\|NCAM | neural cell adhesion molecule 1 | protein-coding |
| chr11 | 112832059 | CD56\|MSK39\|NCAM | neural cell adhesion molecule 1 | protein-coding |
| chr11 | 112832061 | CD56\|MSK39\|NCAM | neural cell adhesion molecule 1 | protein-coding |
| chr11 | 112832102 | CD56\|MSK39\|NCAM | neural cell adhesion molecule 1 | protein-coding |
| chr11 | 113001316 | — | NCAM1 antisense RNA1 | ncRNA |
| chr11 | 113003550 | — | NCAM1 antisense RNA1 | ncRNA |
| chr11 | 113067815 | — | NCAM1 antisense RNA1 | ncRNA |
| chr11 | 113072979 | — | NCAM1 antisense RNA1 | ncRNA |
| chr11 | 113072998 | — | NCAM1 antisense RNA1 | ncRNA |
| chr11 | 113102002 | — | NCAM1 antisense RNA1 | ncRNA |
| chr11 | 113102884 | — | NCAM1 antisense RNA1 | ncRNA |
| chr11 | 113102947 | — | NCAM1 antisense RNA1 | ncRNA |
| chr11 | 113103415 | — | NCAM1 antisense RNA1 | ncRNA |
| chr11 | 113103417 | — | NCAM1 antisense RNA1 | ncRNA |
| chr11 | 113103766 | — | NCAM1 antisense RNA1 | ncRNA |
| chr11 | 113103880 | — | NCAM1 antisense RNA1 | ncRNA |
| chr11 | 113111129 | — | NCAM1 antisense RNA1 | ncRNA |
| chr11 | 113116730 | — | NCAM1 antisense RNA1 | ncRNA |
| chr11 | 113126572 | — | NCAM1 antisense RNA1 | ncRNA |
| chr11 | 113130713 | — | NCAM1 antisense RNA1 | ncRNA |
| chr11 | 113130880 | — | NCAM1 antisense RNA1 | ncRNA |
| chr11 | 113140591 | — | NCAM1 antisense RNA1 | ncRNA |
| chr11 | 113143642 | — | NCAM1 antisense RNA1 | ncRNA |
| chr11 | 133938720 | JAM-2\|JAM-3\|JAM-C\|JAMC | junctional adhesion molecule 3 | protein-coding |
| chr11 | 133938866 | JAM-2\|JAM-3\|JAM-C\|JAMC | junctional adhesion molecule 3 | protein-coding |
| chr11 | 133938880 | JAM-2\|JAM-3\|JAM-C\|JAMC | junctional adhesion molecule 3 | protein-coding |
| chr11 | 133996134 | — | small nucleolar RNA, C/D box 153 | snoRNA |
| chr11 | 134009435 | — | small nucleolar RNA, C/D box 153 | snoRNA |
| chr11 | 134017593 | — | small nucleolar RNA, C/D box 153 | snoRNA |
| chr12 | 7022809 | HEL-S-279\|NSE | enolase 2 | protein-coding |
| chr12 | 7023391 | HEL-S-279\|NSE | enolase 2 | protein-coding |
| chr12 | 7023514 | HEL-S-279\|NSE | enolase 2 | protein-coding |
| chr12 | 7023602 | HEL-S-279\|NSE | enolase 2 | protein-coding |
| chr12 | 7023621 | HEL-S-279\|NSE | enolase 2 | protein-coding |
| chr12 | 7023653 | HEL-S-279\|NSE | enolase 2 | protein-coding |
| chr12 | 7023658 | HEL-S-279\|NSE | enolase 2 | protein-coding |
| chr12 | 7023661 | HEL-S-279\|NSE | enolase 2 | protein-coding |
| chr12 | 7023674 | HEL-S-279\|NSE | enolase 2 | protein-coding |
| chr12 | 7024271 | HEL-S-279\|NSE | enolase 2 | protein-coding |
| chr12 | 7026393 | HEL-S-279\|NSE | enolase 2 | protein-coding |
| chr12 | 7028638 | B37\|D12S755E\|DRPLA\|HRS\|NOD | atrophin 1 | protein-coding |
| chr12 | 7030644 | B37\|D12S755E\|DRPLA\|HRS\|NOD | atrophin 1 | protein-coding |
| chr12 | 7031052 | B37\|D12S755E\|DRPLA\|HRS\|NOD | atrophin 1 | protein-coding |
| chr12 | 24983162 | BCATC\|BCT1\|ECA39\|MECA39\|PNAS121\|PP18 | branched chain amino acid transaminase 1 | protein-coding |
| chr12 | 25055130 | BCATC\|BCT1\|ECA39\|MECA39\|PNAS121\|PP18 | branched chain amino acid transaminase 1 | protein-coding |
| chr12 | 25055222 | BCATC\|BCT1\|ECA39\|MECA39\|PNAS121\|PP18 | branched chain amino acid transaminase 1 | protein-coding |
| chr12 | 25055909 | BCATC\|BCT1\|ECA39\|MECA39\|PNAS121\|PP18 | branched chain amino acid transaminase 1 | protein-coding |
| chr12 | 25101877 | BCATC\|BCT1\|ECA39\|MECA39\|PNAS121\|PP18 | branched chain amino acid transaminase 1 | protein-coding |
| chr12 | 25101883 | BCATC\|BCT1\|ECA39\|MECA39\|PNAS121\|PP18 | branched chain amino acid transaminase 1 | protein-coding |
| chr12 | 25101895 | BCATC\|BCT1\|ECA39\|MECA39\|PNAS121\|PP18 | branched chain amino acid transaminase 1 | protein-coding |
| chr12 | 25101920 | BCATC\|BCT1\|ECA39\|MECA39\|PNAS121\|PP18 | branched chain amino acid transaminase 1 | protein-coding |
| chr12 | 25102293 | BCATC\|BCT1\|ECA39\|MECA39\|PNAS121\|PP18 | branched chain amino acid transaminase 1 | protein-coding |
| chr12 | 25173836 | — | uncharacterized LOC645177 | protein-coding |
| chr12 | 25186593 | JAW1 | lymphoid restricted membrane protein | protein-coding |
| chr12 | 25188314 | JAW1 | lymphoid restricted membrane protein | protein-coding |
| chr12 | 25205089 | JAW1 | lymphoid restricted membrane protein | protein-coding |
| chr12 | 25205105 | JAW1 | lymphoid restricted membrane protein | protein-coding |
| chr12 | 25205106 | JAW1 | lymphoid restricted membrane protein | protein-coding |
| chr12 | 25205346 | JAW1 | lymphoid restricted membrane protein | protein-coding |
| chr12 | 25205421 | JAW1 | lymphoid restricted membrane protein | protein-coding |
| chr12 | 25205476 | JAW1 | lymphoid restricted membrane protein | protein-coding |
| chr12 | 25205499 | JAW1 | lymphoid restricted membrane protein | protein-coding |
| chr12 | 25205534 | JAW1 | lymphoid restricted membrane protein | protein-coding |
| chr12 | 25205574 | JAW1 | lymphoid restricted membrane protein | protein-coding |
| chr12 | 25205779 | JAW1 | lymphoid restricted membrane protein | protein-coding |
| chr12 | 25228574 | JAW1 | lymphoid restricted membrane protein | protein-coding |
| chr12 | 25242608 | JAW1 | lymphoid restricted membrane protein | protein-coding |
| chr12 | 25249767 | JAW1 | lymphoid restricted membrane protein | protein-coding |
| chr12 | 25249792 | JAW1 | lymphoid restricted membrane protein | protein-coding |
| chr12 | 25257163 | JAW1 | lymphoid restricted membrane protein | protein-coding |
| chr12 | 66582559 | ASRT5\|IRAKM | interleukin 1 receptor associated kinase 3 | protein-coding |
| chr12 | 66582875 | ASRT5\|IRAKM | interleukin 1 receptor associated kinase 3 | protein-coding |
| chr12 | 66582937 | ASRT5\|IRAKM | interleukin 1 receptor associated kinase 3 | protein-coding |
| chr12 | 70082891 | VMD2L3 | bestrophin 3 | protein-coding |
| chr12 | 70082956 | VMD2L3 | bestrophin 3 | protein-coding |
| chr12 | 70087752 | VMD2L3 | bestrophin 3 | protein-coding |
| chr12 | 70092965 | VMD2L3 | bestrophin 3 | protein-coding |
| chr12 | 70093015 | VMD2L3 | bestrophin 3 | protein-coding |
| chr12 | 70093024 | VMD2L3 | bestrophin 3 | protein-coding |
| chr12 | 70093031 | VMD2L3 | bestrophin 3 | protein-coding |
| chr12 | 70093041 | VMD2L3 | bestrophin 3 | protein-coding |
| chr12 | 70093044 | VMD2L3 | bestrophin 3 | protein-coding |
| chr12 | 70093075 | VMD2L3 | bestrophin 3 | protein-coding |
| chr12 | 70093096 | VMD2L3 | bestrophin 3 | protein-coding |
| chr12 | 70093156 | VMD2L3 | bestrophin 3 | protein-coding |
| chr12 | 103351364 | ASH1\|HASH1\|MASH1\|bHLHa46 | achaete-scute family bHLH transcription | protein-coding |
| | | | factor 1 | |
| chr13 | 102527544 | FGF-14\|FHF-4\|FHF4\|SCA27 | fibroblast growth factor 14 | protein-coding |
| chr13 | 102568895 | FGF-14\|FHF-4\|FHF4\|SCA27 | fibroblast growth factor 14 | protein-coding |
| chr13 | 103054024 | FGF-14\|FHF-4\|FHF4\|SCA27 | fibroblast growth factor 14 | protein-coding |
| chr14 | 21994247 | COLB\|HSAL2\|Sal-2\|ZNF795\|p150(Sal2) | spalt like transcription factor 2 | protein-coding |
| chr14 | 21994261 | COLB\|HSAL2\|Sal-2\|ZNF795\|p150(Sal2) | spalt like transcription factor 2 | protein-coding |
| chr14 | 21994534 | COLB\|HSAL2\|Sal-2\|ZNF795\|p150(Sal2) | spalt like transcription factor 2 | protein-coding |
| chr14 | 22004936 | COLB\|HSAL2\|Sal-2\|ZNF795\|p150(Sal2) | spalt like transcription factor 2 | protein-coding |
| chr14 | 22004968 | COLB\|HSAL2\|Sal-2\|ZNF795\|p150(Sal2) | spalt like transcription factor 2 | protein-coding |
| chr14 | 22005138 | COLB\|HSAL2\|Sal-2\|ZNF795\|p150(Sal2) | spalt like transcription factor 2 | protein-coding |
| chr14 | 22005250 | COLB\|HSAL2\|Sal-2\|ZNF795\|p150(Sal2) | spalt like transcription factor 2 | protein-coding |
| chr14 | 51448929 | GSD6 | glycogen phosphorylase L | protein-coding |
| chr14 | 51453070 | GSD6 | glycogen phosphorylase L | protein-coding |
| chr14 | 51454994 | GSD6 | glycogen phosphorylase L | protein-coding |
| chr14 | 51561694 | RNF91\|SPRING | tripartite motif containing 9 | protein-coding |
| chr14 | 51561947 | RNF91\|SPRING | tripartite motif containing 9 | protein-coding |
| chr14 | 51562679 | RNF91\|SPRING | tripartite motif containing 9 | protein-coding |
| chr14 | 75988668 | B-ATF\|BATF1\|SFA-2\|SFA2 | basic leucine zipper ATF-like transcription | protein-coding |
| | | | factor | |
| chr14 | 75988698 | B-ATF\|BATF1\|SFA-2\|SFA2 | basic leucine zipper ATF-like transcription | protein-coding |
| | | | factor | |
| chr14 | 75988803 | B-ATF\|BATF1\|SFA-2\|SFA2 | basic leucine zipper ATF-like transcription | protein-coding |
| | | | factor | |
| chr14 | 93389325 | CGA | chromogranin A | protein-coding |
| chr14 | 93389402 | CGA | chromogranin A | protein-coding |
| chr14 | 93389421 | CGA | chromogranin A | protein-coding |
| chr14 | 93389608 | CGA | chromogranin A | protein-coding |
| chr14 | 93392346 | CGA | chromogranin A | protein-coding |
| chr14 | 93397922 | CGA | chromogranin A | protein-coding |
| chr15 | 74465912 | HsT17563\|Meflin | immunoglobulin superfamily containing | protein-coding |
| | | | leucine rich repeat | |
| chr15 | 74466371 | HsT17563\|Meflin | immunoglobulin superfamily containing | protein-coding |
| | | | leucine rich repeat | |
| chr15 | 74466644 | HsT17563\|Meflin | immunoglobulin superfamily containing | protein-coding |
| | | | leucine rich repeat | |
| chr15 | 74466742 | HsT17563\|Meflin | immunoglobulin superfamily containing | protein-coding |
| | | | leucine rich repeat | |
| chr15 | 77285600 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77286921 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77287326 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77287363 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77287365 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77287367 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77287372 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77287413 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77287603 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77287626 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77287653 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77287660 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77287815 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77308052 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77310699 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77319966 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77320584 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77324558 | CD2BP1\|CD2BP1L\|CD2BP1S\|H- | proline-serine-threonine phosphatase | protein-coding |
| | | PIP\|PAPAS\|PSTPIP | interacting protein 1 | |
| chr15 | 77327935 | TM4-A\|TM4SF8\|TSPAN-3 | tetraspanin 3 | protein-coding |
| chr16 | 29674500 | CD43\|GALGP\|GPL115\|LSN | sialophorin | protein-coding |
| chr16 | 29690229 | HEL-S-90n\|QPRTase | quinolinate phosphoribosyltransferase | protein-coding |
| chr16 | 29690258 | HEL-S-90n\|QPRTase | quinolinate phosphoribosyltransferase | protein-coding |
| chr16 | 29690403 | HEL-S-90n\|QPRTase | quinolinate phosphoribosyltransferase | protein-coding |
| chr16 | 29707018 | HEL-S-90n\|QPRTase | quinolinate phosphoribosyltransferase | protein-coding |
| chr16 | 31146861 | CAP1\|PROSTASIN | serine protease 8 | protein-coding |
| chr16 | 31146924 | CAP1\|PROSTASIN | serine protease 8 | protein-coding |
| chr16 | 31146948 | CAP1\|PROSTASIN | serine protease 8 | protein-coding |
| chr16 | 31146983 | CAP1\|PROSTASIN | serine protease 8 | protein-coding |
| chr16 | 74710849 | FANCW\|RNF201 | ring finger and WD repeat domain 3 | protein-coding |
| chr16 | 74712211 | FANCW\|RNF201 | ring finger and WD repeat domain 3 | protein-coding |
| chr16 | 74712746 | FANCW\|RNF201 | ring finger and WD repeat domain 3 | protein-coding |
| chr16 | 74725267 | hMLKL | mixed lineage kinase domain like | protein-coding |
| | | | pseudokinase | |
| chr16 | 74734588 | hMLKL | mixed lineage kinase domain like | protein-coding |
| | | | pseudokinase | |
| chr16 | 74734607 | hMLKL | mixed lineage kinase domain like | protein-coding |
| | | | pseudokinase | |
| chr16 | 74734712 | hMLKL | mixed lineage kinase domain like | protein-coding |
| | | | pseudokinase | |
| chr16 | 74734758 | hMLKL | mixed lineage kinase domain like | protein-coding |
| | | | pseudokinase | |
| chr16 | 88713133 | p22-PHOX | cytochrome b-245 alpha chain | protein-coding |
| chr16 | 88716622 | p22-PHOX | cytochrome b-245 alpha chain | protein-coding |
| chr16 | 88717296 | p22-PHOX | cytochrome b-245 alpha chain | protein-coding |
| chr16 | 88717323 | p22-PHOX | cytochrome b-245 alpha chain | protein-coding |
| chr16 | 88717338 | p22-PHOX | cytochrome b-245 alpha chain | protein-coding |
| chr16 | 88717339 | P22-PHOχ | cytochrome b-245 alpha chain | protein-coding |
| chr16 | 88717343 | p22-PHOX | cytochrome b-245 alpha chain | protein-coding |
| chr16 | 88717349 | P22-PHOX | cytochrome b-245 alpha chain | protein-coding |
| chr16 | 88717362 | p22-PHOX | cytochrome b-245 alpha chain | protein-coding |
| chr16 | 88717460 | p22-PHOX | cytochrome b-245 alpha chain | protein-coding |
| chr17 | 1665153 | EPC-1\|OI12\|OI6\|PEDF\|PIG35 | serpin family F member 1 | protein-coding |
| chr17 | 1665223 | EPC-1\|OI12\|OI6\|PEDF\|PIG35 | serpin family F member 1 | protein-coding |
| chr17 | 1665255 | EPC-1\|OI12\|OI6\|PEDF\|PIG35 | serpin family F member 1 | protein-coding |
| chr17 | 1665262 | EPC-1\|OI12\|OI6\|PEDF\|PIG35 | serpin family F member 1 | protein-coding |
| chr17 | 1665306 | EPC-1\|OI12\|OI6\|PEDF\|PIG35 | serpin family F member 1 | protein-coding |
| chr17 | 1666023 | EPC-1\|OI12\|OI6\|PEDF\|PIG35 | serpin family F member 1 | protein-coding |
| chr17 | 1669468 | EPC-1\|OI12\|OI6\|PEDF\|PIG35 | serpin family F member 1 | protein-coding |
| chr17 | 1673720 | EPC-1\|OI12\|OI6\|PEDF\|PIG35 | serpin family F member 1 | protein-coding |
| chr17 | 1674913 | EPC-1\|OI12\|OI6\|PEDF\|PIG35 | serpin family F member 1 | protein-coding |
| chr17 | 1679644 | EPC-1\|OI12\|OI6\|PEDF\|PIG35 | serpin family F member 1 | protein-coding |
| chr17 | 27283357 | PF1 | PHD finger protein 12 | protein-coding |
| chr17 | 27284731 | PF1 | PHD finger protein 12 | protein-coding |
| chr17 | 27287891 | PF1 | PHD finger protein 12 | protein-coding |
| chr17 | 27308510 | BSRPC | seizure related 6 homolog | protein-coding |
| chr17 | 27308790 | BSRPC | seizure related 6 homolog | protein-coding |
| chr17 | 27332831 | BSRPC | seizure related 6 homolog | protein-coding |
| chr17 | 27332981 | BSRPC | seizure related 6 homolog | protein-coding |
| chr17 | 27333063 | BSRPC | seizure related 6 homolog | protein-coding |
| chr17 | 27333216 | BSRPC | seizure related 6 homolog | protein-coding |
| chr17 | 27333358 | BSRPC | seizure related 6 homolog | protein-coding |
| chr17 | 62007774 | AGM6\|B29\|IGB | CD79b molecule | protein-coding |
| chr17 | 62009521 | AGM6\|B29\|IGB | CD79b molecule | protein-coding |
| chr17 | 62009583 | AGM6\|B29\|IGB | CD79b molecule | protein-coding |
| chr17 | 62009591 | AGM6\|B29\|IGB | CD79b molecule | protein-coding |
| chr17 | 62009596 | AGM6\|B29\|IGB | CD79b molecule | protein-coding |
| chr17 | 62009614 | AGM6\|B29\|IGB | CD79b molecule | protein-coding |
| chr17 | 76970778 | 90K\|BTBD17B\|CyCAP\|M2BP\|MAC-2- | galectin 3 binding protein | protein-coding |
| | | BP\|TANGO10B\|gp90 | | |
| chr17 | 76970979 | 90K\|BTBD17B\|CyCAP\|M2BP\|MAC-2- | galectin 3 binding protein | protein-coding |
| | | BP\|TANGO10B\|gp90 | | |
| chr17 | 76972138 | 90K\|BTBD17B\|CyCAP\|M2BP\|MAC-2- | galectin 3 binding protein | protein-coding |
| | | BP\|TANGO10B\|gp90 | | |
| chr17 | 76973198 | 90K\|BTBD17B\|CyCAP\|M2BP\|MAC-2- | galectin 3 binding protein | protein-coding |
| | | BP\|TANGO10B\|gp90 | | |
| chr17 | 76973305 | 90K\|BTBD17B\|CyCAP\|M2BP\|MAC-2- | galectin 3 binding protein | protein-coding |
| | | BP\|TANGO10B\|gp90 | | |
| chr17 | 76975825 | 90K\|BTBD17B\|CyCAP\|M2BP\|MAC-2- | galectin 3 binding protein | protein-coding |
| | | BP\|TANGO10B\|gp90 | | |
| chr17 | 76975857 | 90K\|BTBD17B\|CyCAP\|M2BP\|MAC-2- | galectin 3 binding protein | protein-coding |
| | | BP\|TANGO10B\|gp90 | | |
| chr17 | 76975873 | 90K\|BTBD17B\|CyCAP\|M2BP\|MAC-2- | galectin 3 binding protein | protein-coding |
| | | BP\|TANGO10B\|gp90 | | |
| chr17 | 76975890 | 90K\|BTBD17B\|CyCAP\|M2BP\|MAC-2- | galectin 3 binding protein | protein-coding |
| | | BP\|TANGO10B\|gp90 | | |
| chr17 | 76975893 | 90K\|BTBD17B\|CyCAP\|M2BP\|MAC-2- | galectin 3 binding protein | protein-coding |
| | | BP\|TANGO10B\|gp90 | | |
| chr17 | 76975900 | 90K\|BTBD17B\|CyCAP\|M2BP\|MAC-2- | galectin 3 binding protein | protein-coding |
| | | BP\|TANGO10B\|gp90 | | |
| chr17 | 76975905 | 90K\|BTBD17B\|CyCAP\|M2BP\|MAC-2- | galectin 3 binding protein | protein-coding |
| | | BP\|TANGO10B\|gp90 | | |
| chr17 | 76975909 | 90K\|BTBD17B\|CyCAP\|M2BP\|MAC-2- | galectin 3 binding protein | protein-coding |
| | | BP\|TANGO10B\|gp90 | | |
| chr17 | 76975917 | 90K\|BTBD17B\|CyCAP\|M2BP\|MAC-2- | galectin 3 binding protein | protein-coding |
| | | BP\|TANGO10B\|gp90 | | |
| chr17 | 76975929 | 90K\|BTBD17B\|CyCAP\|M2BP\|MAC-2- | galectin 3 binding protein | protein-coding |
| | | BP\|TANGO10B\|gp90 | | |
| chr17 | 76976091 | 90K\|BTBD17B\|CyCAP\|M2BP\|MAC-2- | galectin 3 binding protein | protein-coding |
| | | BP\|TANGO10B\|gp90 | | |
| chr18 | 25616449 | CD325\|CDHN\|CDw325\|NCAD | cadherin 2 | protein-coding |
| chr18 | 25625460 | CD325\|CDHN\|CDw325\|NCAD | cadherin 2 | protein-coding |
| chr18 | 25677093 | CD325\|CDHN\|CDw325\|NCAD | cadherin 2 | protein-coding |
| chr18 | 25739161 | CD325\|CDHN\|CDw325\|NCAD | cadherin 2 | protein-coding |
| chr18 | 25757310 | CD325\|CDHN\|CDw325\|NCAD | cadherin 2 | protein-coding |
| chr18 | 31158479 | BRPS\|KIAA1713 | ASXL transcriptional regulator 3 | protein-coding |
| chr18 | 31158496 | BRPS\|KIAA1713 | ASXL transcriptional regulator 3 | protein-coding |
| chr18 | 31158505 | BRPS\|KIAA1713 | ASXL transcriptional regulator 3 | protein-coding |
| chr18 | 31158508 | BRPS\|KIAA1713 | ASXL transcriptional regulator 3 | protein-coding |
| chr18 | 31158530 | BRPS\|KIAA1713 | ASXL transcriptional regulator 3 | protein-coding |
| chr18 | 31158635 | BRPS\|KIAA1713 | ASXL transcriptional regulator 3 | protein-coding |
| chr18 | 31158746 | BRPS\|KIAA1713 | ASXL transcriptional regulator 3 | protein-coding |
| chr18 | 31185450 | BRPS\|KIAA1713 | ASXL transcriptional regulator 3 | protein-coding |
| chr18 | 31185451 | BRPS\|KIAA1713 | ASXL transcriptional regulator 3 | protein-coding |
| chr18 | 31185458 | BRPS\|KIAA1713 | ASXL transcriptional regulator 3 | protein-coding |
| chr18 | 31185463 | BRPS\|KIAA1713 | ASXL transcriptional regulator 3 | protein-coding |
| chr18 | 31185472 | BRPS\|KIAA1713 | ASXL transcriptional regulator 3 | protein-coding |
| chr18 | 31185490 | BRPS\|KIAA1713 | ASXL transcriptional regulator 3 | protein-coding |
| chr18 | 31187471 | BRPS\|KIAA1713 | ASXL transcriptional regulator 3 | protein-coding |
| chr19 | 39989435 | SCDO1 | delta like canonical Notch ligand 3 | protein-coding |
| chr19 | 39989507 | SCDO1 | delta like canonical Notch ligand 3 | protein-coding |
| chr19 | 39989508 | SCDO1 | delta like canonical Notch ligand 3 | protein-coding |
| chr19 | 46916741 | 3M3\|PPP1R20\|p90 | coiled-coil domain containing 8 | protein-coding |
| chr19 | 51358071 | APS\|KLK2A1\|PSA\|hK3 | kallikrein related peptidase 3 | protein-coding |
| chr19 | 51358077 | APS\|KLK2A1\|PSA\|hK3 | kallikrein related peptidase 3 | protein-coding |
| chr19 | 51358086 | APS\|KLK2A1\|PSA\|hK3 | kallikrein related peptidase 3 | protein-coding |
| chr19 | 51358091 | APS\|KLK2A1\|PSA\|hK3 | kallikrein related peptidase 3 | protein-coding |
| chr19 | 51358105 | APS\|KLK2A1\|PSA\|hK3 | kallikrein related peptidase 3 | protein-coding |
| chr19 | 51358112 | APS\|KLK2A1\|PSA\|hK3 | kallikrein related peptidase 3 | protein-coding |
| chr19 | 51358128 | APS\|KLK2A1\|PSA\|hK3 | kallikrein related peptidase 3 | protein-coding |
| chr19 | 51360547 | APS\|KLK2A1\|PSA\|hK3 | kallikrein related peptidase 3 | protein-coding |
| chr20 | 3210322 | BTR1\|CDPD1\|CHED\|CHED2\|NABC1\|dJ794I6.2 | solute carrier family 4 member 11 | protein-coding |
| chr20 | 3211106 | BTR1\|CDPD1\|CHED\|CHED2\|NABC1\|dJ794I6.2 | solute carrier family 4 member 11 | protein-coding |
| chr20 | 3218273 | BTR1\|CDPD1\|CHED\|CHED2\|NABC1\|dJ794I6.2 | solute carrier family 4 member 11 | protein-coding |
| chr20 | 3218712 | BTR1\|CDPD1\|CHED\|CHED2\|NABC1\|dJ794I6.2 | solute carrier family 4 member 11 | protein-coding |
| chr20 | 3219259 | BTR1\|CDPD1\|CHED\|CHED2\|NABC1\|dJ794I6.2 | solute carrier family 4 member 11 | protein-coding |
| chr20 | 3219736 | BTR1\|CDPD1\|CHED\|CHED2\|NABC1\|dJ794I6.2 | solute carrier family 4 member 11 | protein-coding |
| chr20 | 5891976 | SCG1 | chromogranin B | protein-coding |
| chr20 | 5892076 | SCG1 | chromogranin B | protein-coding |
| chr20 | 5896044 | SCG1 | chromogranin B | protein-coding |
| chr20 | 20348665 | IA-1\|IA1 | INSM transcriptional repressor 1 | protein-coding |
| chr20 | 22565001 | HNF3B\|TCF3B | forkhead box A2 | protein-coding |
| chr20 | 22565993 | HNF3B\|TCF3B | forkhead box A2 | protein-coding |
| chr21 | 42839929 | IFI-78K\|IFI78\|MX\|MxA | MX dynamin like GTPase 1 | protein-coding |
| chr21 | 42840664 | IFI-78K\|IFI78\|MX\|MxA | MX dynamin like GTPase 1 | protein-coding |
| chr21 | 42878431 | PP9284\|PRSS10 | transmembrane serine protease 2 | protein-coding |
| chr21 | 42879809 | PP9284\|PRSS10 | transmembrane serine protease 2 | protein-coding |
| chr21 | 42879892 | PP9284\|PRSS10 | transmembrane serine protease 2 | protein-coding |
| chr21 | 42879919 | PP9284\|PRSS10 | transmembrane serine protease 2 | protein-coding |
| chr21 | 42879941 | PP9284\|PRSS10 | transmembrane serine protease 2 | protein-coding |
| chr21 | 42879985 | PP9284\|PRSS10 | transmembrane serine protease 2 | protein-coding |
| chr21 | 42879986 | PP9284\|PRSS10 | transmembrane serine protease 2 | protein-coding |
| chr21 | 42902925 | PP9284\|PRSS10 | transmembrane serine protease 2 | protein-coding |
| chr21 | 42902943 | PP9284\|PRSS10 | transmembrane serine protease 2 | protein-coding |
| chrX | 49056535 | MRX96\|MRXSYP | synaptophysin | protein-coding |
| chrX | 49056552 | MRX96\|MRXSYP | synaptophysin | protein-coding |
| chrX | 49056554 | MRX96\|MRXSYP | synaptophysin | protein-coding |
| chrX | 49056558 | MRX96\|MRXSYP | synaptophysin | protein-coding |
| chrX | 49056618 | MRX96\|MRXSYP | synaptophysin | protein-coding |
| chrX | 66764365 | AIS\|AR8\|DHTR\|HUMARA\|HYSP1\|KD\|NR3C4\| | androgen receptor | protein-coding |
| | | SBMA\|SMAX1\|TFM | | |
| chrX | 66764562 | AIS\|AR8\|DHTR\|HUMARA\|HYSP1\|KD\|NR3C4\| | androgen receptor | protein-coding |
| | | SBMA\|SMAX1\|TFM | | |
| chrX | 66764886 | AIS\|AR8\|DHTR\|HUMARA\|HYSP1\|KD\|NR3C4\| | androgen receptor | protein-coding |
| | | SBMA\|SMAX1\|TFM | | |
| chrX | 66788631 | AIS\|AR8\|DHTR\|HUMARA\|HYSP1\|KD\|NR3C4\| | androgen receptor | protein-coding |
| | | SBMA\|SMAX1\|TFM | | |
| chr1 | 202039153 | ET(B)R-LP-2\|ETBR-LP-2\|ETBRLP2 | G protein-coupled receptor 37 like 1 | protein-coding |
| chr1 | 241206098 | mir-3123 | microRNA 3123 | ncRNA |
| chr2 | 89530057 | mir-4436a | microRNA 4436a | ncRNA |
| chr3 | 193490472 | — | long intergenic non-protein coding RNA | ncRNA |
| | | | 2038 | |
| chr5 | 1548487 | AGPAT10\|AGPAT9\|AYTL2\|LPCAT- | lysophosphatidylcholine acyltransferase 1 | protein-coding |
| | | 1\|PFAAP3\|lpcat\|lysoPAFAT | | |
| chr5 | 36521979 | EA6\|EAAT1\|GLAST\|GLAST1 | solute carrier family 1 member 3 | protein-coding |
| chr5 | 39390555 | ARMD15\|C9D | complement C9 | protein-coding |
| chr5 | 71146894 | CART | CART prepropeptide | protein-coding |
| chr5 | 142194123 | GRAF\|GRAF1\|OPHN1L\|OPHN1L1 | Rho GTPase activating protein 26 | protein-coding |
| chr5 | 168573593 | MIRN585\|hsa-mir-585\|mir-585 | microRNA 585 | ncRNA |
| chr7 | 2006579 | MAD1\|PIG9\|TP53I9\|TXBP181 | mitotic arrest deficient 1 like 1 | protein-coding |
| chr7 | 7799018 | REPA3\|RP-A p14 | replication protein A3 | protein-coding |
| chr7 | 127841628 | MIR-129\|bMIRN129-1\|mir-129-1 | microRNA 129-1 | ncRNA |
| chr7 | 129912398 | — | carboxypeptidase A2 | protein-coding |
| chr8 | 53499560 | AUGA\|AUGB\|FAM150A\|UNQ9433 | ALK and LTK ligand 1 | protein-coding |
| chr8 | 61990390 | — | hCG1816373-like | ncRNA |
| chr8 | 69087227 | VEST-1\|VEST1 | chromosome 8 open reading frame 34 | protein-coding |
| chr8 | 73479990 | KV2.2 | potassium voltage-gated channel subfamily | protein-coding |
| | | | B member 2 | |
| chr8 | 73849054 | PIN2\|TRBF1\|TRF\|TRF1\|hTRF1-AS\|t-TRF1 | telomeric repeat binding factor 1 | protein-coding |
| chr8 | 133070961 | PLA2L | otoconin 90 | protein-coding |
| chr8 | 140727998 | K2p9.1\|KT3.2\|TASK-3\|TASK3 | potassium two pore domain channel | protein-coding |
| | | | subfamily K member 9 | |
| chr8 | 141458926 | IBP\|IKBKBBP\|MRT13\|NIBP\|T1\|TRS120 | trafficking protein particle complex 9 | protein-coding |
| chr8 | 141994084 | FADK\|FAK\|FAK1\|FRNK\|PPP1R71\|p125FAK\| | protein tyrosine kinase 2 | protein-coding |
| | | pp125FAK | | |
| chr8 | 142367954 | — | G protein-coupled receptor 20 | protein-coding |
| chr8 | 143395944 | — | t-SNARE domain containing 1 | protein-coding |
| chr9 | 36748433 | — | microRNA 4475 | ncRNA |
| chr9 | 38437314 | IGFBP-RP4\|IGFBPRP4\|bA113O24.1 | insulin like growth factor binding protein | protein-coding |
| | | | like 1 | |
| chr9 | 93792388 | — | uncharacterized LOC100129316 | ncRNA |
| chr9 | 133769512 | 26RFa\|P518 | pyroglutamylated RFamide peptide | protein-coding |
| chr9 | 134913164 | CRAP34\|CRSP34\|CRSP8\|MED3\|TRAP37 | mediator complex subunit 27 | protein-coding |
| chr9 | 138719304 | — | calmodulin regulated spectrin associated | protein-coding |
| | | | protein 1 | |
| chr14 | 100039425 | — | coiled-coil domain containing 85C | protein-coding |
| chr18 | 8660098 | MTCL1-AS1\|MTCL1AS1 | gastric cancer associated transcript 2 | ncRNA |
| chr18 | 14917016 | — | long intergenic non-protein coding RNA | ncRNA |
| | | | 1443 | |
| chr18 | 25442246 | CD325\|CDHN\|CDw325\|NCAD | cadherin 2 | protein-coding |
| chr18 | 47258089 | DSAEC | acetyl-CoA acyltransferase 2 | protein-coding |
| chr20 | 20421462 | IA-1\|IA1 | INSM transcriptional repressor 1 | protein-coding |
| chr20 | 21563921 | — | long intergenic non-protein coding RNA | ncRNA |
| | | | 1727 | |
| chr21 | 32546326 | TIAM-1 | T cell lymphoma invasion and metastasis 1 | protein-coding |
| chr21 | 46885392 | KNO\|KNO1\|KS | collagen type XVIII alpha 1 chain | protein-coding |

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments encompassed by the present invention described herein. Such equivalents are intended to be encompassed by the following claims.

What is claimed is:

1. A method of assessing whether a subject is afflicted with castration-resistant neuroendocrine prosate cancer (CRPC-NE) or at risk for developing CRPC-NE, the method comprising determining the presence or absence of one or more genomic or epigenomic alterations selected from:
i) deletion or mutation of at least one biomarker listed in Table 1A;
ii) gain or mutation of at least one biomarker listed in Table 1B;
iii) hypermethylation of at least one genomic site listed in Table 1C; and
iv) hypomethylation of at least one genomic site listed in Table 1D in the genomic DNA;
wherein the presence of deletion or mutation of at least one biomarker listed in Table 1A, hypermethylation of at least one genomic site listed in Table 1C, and/or hypomethylation of at least one genomic site listed in Table 1D in the genomic DNA; and/or the absence of gain or mutation of at least one biomarker listed in Table 1B in the genomic DNA isolated from the biological sample indicates that the subject is afflicted with CRPC-NE or at risk for developing CRPC-NE, optionally obtaining a biological sample from the subject for the determination step.

2. The method of claim 1, wherein the deletion or mutation of at least one biomarker listed in Table 1A or the gain or mutation of at least one biomarker listed in Table 1B is detected by whole exome sequencing (WES).

3. The method of claim 1 or 2, wherein the hypermethylation of at least one genomic site listed in Table 1C or hypomethylation of at least one genomic site listed in Table 1D is detected by whole genome bisulfite sequencing (WGBS).

4. The method of any one of claims 1-3, wherein the deletion of at least one biomarker listed in Table 1A is a homozygous deletion, a heterozygous deletion, a copy number neutral loss, or an event defined by loss of one allele and gain of the other allele.

5. The method of any one of claims 1-4, wherein the mutation of at least one biomarker listed in Table 1A is a non-synonymous single-nucleotide variant (SNV).

6. The method of any one of claims 1-5, wherein the gain of at least one biomarker listed in Table 1B is a focal gain.

7. The method of any one of claims 1-6, wherein the mutation of at least one biomarker listed in Table 1B is a non-synonymous single-nucleotide variant (SNV).

8. The method of claim 7, wherein the mutation of at least one biomarker listed in Table 1B is L702H or T878A of SEQ ID NO: 48.

9. The method of any one of claims 1-8, wherein the hypermethylation of at least one genomic site listed in Table 1C is defined by a higher methylation level than the site specific tissue-based threshold.

10. The method of any one of claims 1-9, wherein the hypomethylation of at least one genomic site listed in Table 1C is defined by a lower methylation level than the site specific tissue-based threshold.

11. The method of claim 9 or 10, wherein the threshold for each genomic site listed in Table 1C or 1D is a pre-determined threshold that discriminates between castration resistant prostate adenocarcinoma (CRPC-Adeno) and CRPC-NE tissue samples.

12. The method of any one of claims 1-11, further comprising, after step (c), calculating a neuroendocrine prostate cancer (NEPC) score based on the presence or absence of one or more genomic or epigenomic features determined in step (c).

13. The method of claim 12, further comprising comparing the NEPC score to a control, wherein a higher NEPC score compared to the control indicates that the subject is afflicted with CRPC-NE or at risk for developing CRPC-NE.

14. The method of claim 13, wherein the control is a reference value.

15. The method of claim 13, wherein the control is a NEPC score determined from a control sample.

16. The method of claim 15, wherein the control sample is obtained from a subject without CRPC-NE, or a member of the same species to which the subject belongs without CRPC-NE.

17. The method of claim 15 or 16, wherein the control sample is obtained from a subject with castration resistant prostate adenocarcinoma (CRPC-Adeno).

18. The method of any one of claims 1-17, wherein the sample is selected from the group consisting of organs, tissue, body fluids and cells.

19. The method of any one of claims 1-18, wherein the body fluid is selected from the group consisting of whole blood, serum, plasma, sputum, spinal fluid, lymph fluid, skin secretions, respiratory secrections, intestinal secretions, genitourninary tract secretions, tears, milk, buccal scrape, saliva, cerebrospinal fluid, urine, and stool.

20. The method of any one of claims 1-19, wherein the sample is whole blood, serum or plasma.

21. The method of any one of claims 1-20, wherein cell-free DNA (cfDNA) or circulating tumore DNA (ctDNA) isolated from plasma is used for the determination.

22. The method of any one of claims 1-21, wherein genomic DNA isolated from a tumor cell or tissue is used for the determination.

23. The method of any one of claims 1-22, further comprising comparing additional biomarkers for CRPC-NE.

24. The method of any one of claims 1-23, wherein the additional biomarker is selected from the group consisting of AR, a downstream AR-regulated marker, and a classical neuroendocrine marker.

25. The method of claim 24, wherein the downstream AR-regulated marker is selected from the group consisting of prostate specific antigen (PSA), NKX3.1, and TMPRSS2.

26. The method of claim 24, wherein the classical neuroendocrine marker is selected from the group consisting of chromogranin, synaptophysin, neuron specific enolase, and CD56.

27. The method of any one of claims 1-26, further comprising detecting morphological features of a tumor biopsy from the subject.

28. The method of any one of claims 1-27, wherein the subject is afflicted with castration-resistant prostate cancer (CRPC).

29. The method of any one of claims 1-28, wherein the subject is resistant to an androgen receptor (AR)-directed therapy.

30. The method of any one of claims 1-29, further comprising administering to the subject an anti-cancer therapy other than an AR-targeted therapy as a single agent if the subject is afflicted with CRPC-NE or at risk for developing CRPC-NE.

31. The method of claim 30, wherein the anti-cancer therapy is selected from the group consisting of an epgenitic modifier, targeted therapy, chemotherapy, radiation therapy, and/or hormonal therapy, optionally wherein the anti-cancer therapy comprises an AR-targeted therapy.

32. The method of claim 30 or 31, wherein the anti-cancer therapy is administered to the subject in combination with the AR-targeted therapy, optionally wherein the anti-cancer therapy is administered before, after, or concurrently with the AR-targeted therapy.

33. The method of claim 31, wherein the targeted therapy is an immunotherapy.

34. The method of claim 33, wherein the immunotherapy is cell-based.

35. The method of claim 33, wherein the immunotherapy comprises a cancer vaccine and/or virus.

36. The method of claim 33, wherein the immunotherapy inhibits an immune checkpoint.

37. The method of claim 36, wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR.

38. The method of claim 37, wherein the immune checkpoint is PD1, PD-L1, or CTLA-4.

39. The method of any one of claims 30-32, wherein the anti-cancer therapy is a platinum-based chemotherapy.

40. A method for monitoring the progression of CRPC in a subject, the method comprising:
a) detecting in a subject sample at a first point in time the presence or absence of one or more genomic or epigenomic features selected from:
i) deletion or mutation of at least one biomarker listed in Table 1A;
ii) gain or mutation of at least one biomarker listed in Table 1B;
iii) hypermethylation of at least one genomic site listed in Table 1C; and
iv) hypomethylation of at least one genomic site listed in Table 1D;
b) calculating a first NEPC score based on the presence or absence of one or more genomic or epigenomic features determined in step a);
c) repeating step a) at a subsequent point in time;
d) calculating a second NEPC score based on the presence or absence of one or more genomic or epigenomic features determined in step c); and
e) comparing the NEPC scores determined in steps b) and d), and therefrom monitoring the progression of CRPC in the subject.

41. A method of assessing the efficacy of an agent for treating CRPC-NE in a subject, the method comprising:
a) detecting in a subject sample at a first point in time the presence or absence of one or more genomic or epigenomic features selected from:
i) deletion or mutation of at least one biomarker listed in Table 1A;
ii) gain or mutation of at least one biomarker listed in Table 1B;
iii) hypermethylation of at least one genomic site listed in Table 1C; and
iv) hypomethylation of at least one genomic site listed in Table 1D;
b) calculating a first NEPC score based on the presence or absence of one or more genomic or epigenomic features determined in step a);
c) repeating step a) during at least one subsequent point in time after administration of the agent;
d) calculating a second NEPC score based on the presence or absence of one or more genomic or epigenomic features determined in step c); and
e) comparing the NEPC scores determined from steps b) and d),
wherein a higher NEPC score determined in the subsequent sample, relative to the sample at the first point in time, indicates that the agent does not treat CRPC-NE in the subject; and
wherein a lower NEPC score determined in the subsequent sample, relative to the sample at the first point in time, indicates that the agent treats CRPC-NE in the subject.

42. The method of claim 40 or 41, wherein between the first point in time and the subsequent point in time, the subject has undergone treatment, completed treatment, and/or is in remission for CRPC-NE.

43. The method of any one of claims 40-42, wherein the first and/or at least one subsequent sample is selected from the group consisting of ex vivo and in vivo samples.

44. The method of any one of claims 40-43, wherein the first and/or at least one subsequent sample is obtained from an animal model of CRPC-NE.

45. The method of any one of claims 40-44, wherein the first and/or at least one subsequent sample is a portion of a single sample or pooled samples obtained from the subject.

46. The method of any one of claims 40-45, wherein the sample comprises cells, cell lines, histological slides, paraffin embedded tissue, fresh frozen tissue, fresh tissue, biopsies, blood, plasma, serum, buccal scrape, saliva, cerebrospinal fluid, urine, stool, mucus, bone marrow, peritumoral tissue, and/or intratumoral tissue obtained from the subject.

47. The method of any one of claims 40-46, wherein the sample is whole blood, serum or plasma.

48. The method of claim 46, wherein the sample is a tumor cell or tissue, and genomic DNA is isolated from the plasma and used for detecting the presence or absence of one or more genomic or epigenomic features.

49. The method of claim 47, wherein the sample is plasma, and cell-free DNA (cfDNA) or circulating tumore DNA (ctDNA) is isolated from the plasma and used for detecting the presence or absence of one or more genomic or epigenomic features.

50. A cell-based assay for screening for agents that have a cytotoxic or cytostatic effect on a CRPC-NE cancer cell comprising, contacting the CRPC-NE cancer cell with a test agent, and determining the ability of the teat agent: i) to inhibit deletion or mutation of at least one biomarker listed in Table 1A; ii) to induce gain or mutation of at least one biomarker listed in Table 1B; iii) to decrease the methylation level at least one genomic site listed in Table 1C; and/or iv) to increase the methylation level of at least one genomic site listed in Table 1D in the subject sample.

51. The cell-based assay of claim 52, wherein the step of contacting occurs in vivo, ex vivo, or in vitro.

52. The cell-based assay of claim 51 or 52, further comprising administering the test agent to an animal model of CRPC-NE.

53. A kit for assessing the ability of a agent to treat CRPC-NE, the kit comprising a reagent for assessing the presence or absence of one or more genomic or epigenomic features selected from:
i) deletion or mutation of at least one biomarker listed in Table 1A;
ii) gain or mutation of at least one biomarker listed in Table 1B;
iii) hypermethylation of at least one genomic site listed in Table 1C; and
iv) hypomethylation of at least one genomic site listed in Table 1D.

54. A kit for assessing whether a subject is afflicted with CRPC-NE or at risk for developing CRPC-NE, the kit comprising a reagent for assessing the presence or absence of one or more genomic or epigenomic features selected from:
i) deletion or mutation of at least one biomarker listed in Table 1A;
ii) gain or mutation of at least one biomarker listed in Table 1B;
iii) hypermethylation of at least one genomic site listed in Table 1C; and
iv) hypomethylation of at least one genomic site listed in Table 1D.

55. A method of treating a subject afflicted with CRPC-NE comprising administering to the subject a therapeutically effective amount of an agent that modulates the presence or absence of one or more genomic or epigenomic features selected from:
i) deletion or mutation of at least one biomarker listed in Table 1A;
ii) gain or mutation of at least one biomarker listed in Table 1B;
iii) hypermethylation of at least one genomic site listed in Table 1C; and
iv) hypomethylation of at least one genomic site listed in Table 1D.

56. The method of claim 55, wherein the agent inhibits deletion or mutation of at least one biomarker listed in Table 1A, thereby treating a subject afflicted with CRPC-NE.

57. The method of claim 55, wherein the agent induces gain or mutation of at least one biomarker listed in Table 1B, thereby treating a subject afflicted with CRPC-NE.

58. The method of claim 55, wherein the agent decreases the methylation level at least one genomic site listed in Table 1C, thereby treating a subject afflicted with CRPC-NE.

59. The method of claim 55, wherein the agent increases the methylation level at least one genomic site listed in Table 1D, thereby treating a subject afflicted with CRPC-NE.

60. The method of claim 58 or 59, wherein the agent is an epigenetic modifier.

61. The method of claim 60, wherein the epigenetic modifier is an EZH2 inhibitor.

62. The method of any one of claims 55-61, further comprising administering to the subject an immunotherapy and/or cancer therapy, optionally wherein the immunotherapy and/or cancer therapy is administered before, after, or concurrently with the agent.

63. The method of claim 62, wherein the immunotherapy is cell-based.

64. The method of claim 62, wherein the immunotherapy comprises a cancer vaccine and/or virus.

65. The method of claim 62, wherein the immunotherapy inhibits an immune checkpoint.

66. The method of claim 65, wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR.

67. The method of claim 66, wherein the immune checkpoint is PD1, PD-L1, or CD47.

68. The method of claim 62, wherein the cancer therapy is selected from the group consisting of radiation, a radiosensitizer, and a chemotherapy.

69. The method of claim 68, wherein the cancer therapy is a platinum-based chemotherapy.

70. The method or assay of any one of claims 40-69, wherein the deletion or mutation of at least one biomarker listed in Table 1A or the gain or mutation of at least one biomarker listed in Table 1B is detected by whole exome sequencing (WES).

71. The method or assay of any one of claims 40-70, wherein the hypermethylation of at least one genomic site listed in Table 1C or hypomethylation of at least one genomic site listed in Table 1D is detected by whole genome bisulfate sequencing (WGBS).

72. The method or assay of any one of claims 40-71, wherein the deletion of at least one biomarker listed in Table 1A is a homozygous deletion, a heterozygous deletion, a copy number neutral loss, or an event defined by loss of one allele and gain of the other allele.

73. The method or assay of any one of claims 40-72, wherein the mutation of at least one biomarker listed in Table 1A is a non-synonymous single-nucleotide variant (SNV).

74. The method or assay of any one of claims 40-73, wherein the gain of at least one biomarker listed in Table 1B is a focal gain.

75. The method or assay of any one of claims 40-74, wherein the mutation of at least one biomarker listed in Table 1B is a non-synonymous single-nucleotide variant (SNV).

76. The method or assay of claim 75, wherein the mutation of at least one biomarker listed in Table 1B is L702H or T878A of SEQ ID NO: 48.

77. The method or assay of any one of claims 40-76, wherein the hypermethylation of at least one genomic site listed in Table 1C is defined by a higher methylation level than the site specific tissue-based threshold.

78. The method or assay of any one of claims 40-77, wherein the hypomethylation of at least one genomic site listed in Table 1C is defined by a lower methylation level than the site specific tissue-based threshold.

79. The method or assay of any one of claims 40-78, wherein the threshold for each genomic site listed in Table 1C or 1D is the threshold that best discriminates between castration resistant prostate adenocarcinoma (CRPC-Adeno) and CRPC-NE tissue samples by Receiver operating characteristic (ROC) curve analysis.

80. A method of assessing whether a subject is afflicted with or at risk for developing castration-resistant neuroendocrine prostate cancer (CRPC-NE) or castration-resistant adenocarcinoma prostate cancer (CRPC-Adeno), the method comprising determining the presence or absence of one or more genomic or epigenomic alterations in at least one biomarker listed in Table 14,
wherein the presence one or more genomic or epigenomic alterations listed in Table 14 in the genomic DNA isolated from the biological sample indicates that the subject is afflicted with CRPC-NE or CRPC-Adeno or at risk for developing CRPC-NE or CRPC-Adeno, optionally obtaining a biological sample from the subject for the determination step.

81. The method of claim 80, wherein the one or more genomic or epigenomic alterations listed in Table 14 comprises a mutation detected by whole exome sequencing (WES).

82. The method of claim 80, wherein the one or more genomic or epigenomic alterations listed in Table 14 comprises hypermethylation or hypomethylation of at least one genomic site listed in Table 14 detected by whole genome bisulfite sequencing (WGBS).

83. The method of claim 80, wherein the one or more genomic or epigenomic alterations listed in Table 14 comprise a homozygous deletion, a heterozygous deletion, a copy number neutral loss, or an event defined by loss of one allele and gain of the other allele.

84. The method of claim 80, wherein the one or more genomic or epigenomic alterations listed in Table 14 is a non-synonymous single-nucleotide variant (SNV).

85. The method of claim 80, wherein the one or more genomic or epigenomic alterations listed in Table 14 is a focal gain of at least one biomarker listed in Table 14

86. The method of claim 80, wherein the one or more genomic or epigenomic alterations listed in Table 14 is a non-synonymous single-nucleotide variant (SNV).

87. The method of claim 82, wherein the hypermethylation of at least one genomic site listed in Table 14 is defined by a higher methylation level than the site specific tissue-based threshold.

88. The method of claim 82, wherein the hypomethylation of at least one genomic site listed in Table 14 is defined by a lower methylation level than the site specific tissue-based threshold.

89. The method of claim 87 or 88, wherein the threshold for each genomic site listed in Table 14 is a pre-determined threshold that discriminates between castration resistant prostate adenocarcinoma (CRPC-Adeno) and CRPC-NE tissue samples.

90. The method of any one of claims 80-89 further comprising, after step (c), calculating a neuroendocrine prostate cancer (NEPC) score based on the presence or absence of one or more genomic or epigenomic features determined in step (c).

91. The method of claim 90, further comprising comparing the NEPC score to a control, wherein a higher NEPC score compared to the control indicates that the subject is afflicted with CRPC-NE or at risk for developing CRPC-NE.

92. The method of claim 91, wherein the control is a reference value.

93. The method of claim 91, wherein the control is a NEPC score determined from a control sample.

94. The method of claim 93, wherein the control sample is obtained from a subject without CRPC-NE, or a member of the same species to which the subject belongs without CRPC-NE.

95. The method of claim 93 or 94, wherein the control sample is obtained from a subject with castration resistant prostate adenocarcinoma (CRPC-Adeno).

96. The method of any one of claims 80-95, wherein the sample is selected from the group consisting of organs, tissue, body fluids and cells.

97. The method of any one of claims 80-96, wherein the body fluid is selected from the group consisting of whole blood, serum, plasma, sputum, spinal fluid, lymph fluid, skin secretions, respiratory secrections, intestinal secretions, genitourninary tract secretions, tears, milk, buccal scrape, saliva, cerebrospinal fluid, urine, and stool.

98. The method of any one of claims 80-97, wherein the sample is whole blood, serum or plasma.

99. The method of any one of claims 80-98, wherein cell-free DNA (cfDNA) or circulating tumore DNA (ctDNA) isolated from plasma is used for the determination.

100. The method of any one of claims 80-99, wherein genomic DNA isolated from a tumor cell or tissue is used for the determination.

101. The method of any one of claims 80-100, further comprising comparing additional biomarkers for CRPC-NE.

102. The method of any one of claims 80-101, wherein the additional biomarker is selected from the group consisting of AR, a downstream AR-regulated marker, and a classical neuroendocrine marker.

103. The method of claim 102, wherein the downstream AR-regulated marker is selected from the group consisting of prostate specific antigen (PSA), NKX3.1, and TMPRSS2.

104. The method of claim 102, wherein the classical neuroendocrine marker is selected from the group consisting of chromogranin, synaptophysin, neuron specific enolase, and CD56.

105. The method of any one of claims 80-104, further comprising detecting morphological features of a tumor biopsy from the subject.

106. The method of any one of claims 80-105, wherein the subject is afflicted with castration-resistant prostate cancer (CRPC).

107. The method of any one of claims 80-106, wherein the subject is resistant to an androgen receptor (AR)-directed therapy.

108. The method of any one of claims 80-107, further comprising administering to the subject an anti-cancer therapy other than an AR-targeted therapy as a single agent if the subject is afflicted with CRPC-NE or at risk for developing CRPC-NE.

109. The method of claim 108, wherein the anti-cancer therapy is selected from the group consisting of an epgenitic modifier, targeted therapy, chemotherapy, radiation therapy, and/or hormonal therapy, optionally wherein the anti-cancer therapy comprises an AR-targeted therapy.

110. The method of claim 108 or 109, wherein the anti-cancer therapy is administered to the subject in combination with the AR-targeted therapy, optionally wherein the anti-cancer therapy is administered before, after, or concurrently with the AR-targeted therapy.

111. The method of claim 110, wherein the targeted therapy is an immunotherapy.

112. The method of claim 111, wherein the immunotherapy is cell-based.

113. The method of claim 111, wherein the immunotherapy comprises a cancer vaccine and/or virus.

114. The method of claim 111, wherein the immunotherapy inhibits an immune checkpoint.

115. The method of claim 114, wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR.

116. The method of claim 114, wherein the immune checkpoint is PD1, PD-L1, or CTLA-4.

117. The method of any one of claims 114-116, wherein the anti-cancer therapy is a platinum-based chemotherapy.

118. The method or assay of any one of claims 1-117, wherein the agent is administered in a pharmaceutically acceptable formulation.

119. The method or assay of any one of claims 1-118, wherein the subject is an animal model of CRPC-NE.

120. The method or assay of claim 119, wherein the animal model is a rodent model.

121. The method or assay of any one of claims 1-119, wherein the subject is a mammal.

122. The method or assay of any one of claims 1-121, wherein the mammal is a mouse or a human.

123. The method or assay of any one of claims 1-122, wherein the mammal is a human.


SEQUENCE LISTING (59 sequences)

SEQ ID NO: 1 - protein, 393 aa, Homo sapiens [sequence omitted]

SEQ ID NO: 2 - DNA, 2591 bp, Homo sapiens
gatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 3 - DNA, 2588 bp, Homo sapiens
gatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 4 - protein, 341 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC

SEQ ID NO: 5 - DNA, 2724 bp, Homo sapiens
gatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcaggaccagaccagctttcaaaaagaaaattgttaaagagagcatgaaaatggttctatgactttgcctgatacagatgctacttgacttacgatggtgttacttcctgataaactcgtcgtaagttgaaaatattatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 6 - protein, 346 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQMLLDLRWCYFLINSS

SEQ ID NO: 7 - DNA, 2651 bp, Homo sapiens
gatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatgctacttgacttacgatggtgttacttcctgataaactcgtcgtaagttgaaaatattatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 8 - protein, 261 aa, Homo sapiens
MFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 9 - DNA, 2271 bp, Homo sapiens
tgaggccaggagatggaggctgcagtgagctgtgatcacaccactgtgctccagcctgagtgacagagcaagaccctatctcaaaaaaaaaaaaaaaaaagaaaagctcctgaggtgtagacgccaactctctctagctcgctagtgggttgcaggaggtgcttacgcatgtttgtttctttgctgccgtcttccagttgctttatctgttcacttgtgccctgactttcaactctgtctccttcctcttcctacagtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 10 - protein, 209 aa, Homo sapiens
MFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC

SEQ ID NO: 11 - DNA, 2404 bp, Homo sapiens
tgaggccaggagatggaggctgcagtgagctgtgatcacaccactgtgctccagcctgagtgacagagcaagaccctatctcaaaaaaaaaaaaaaaaaagaaaagctcctgaggtgtagacgccaactctctctagctcgctagtgggttgcaggaggtgcttacgcatgtttgtttctttgctgccgtcttccagttgctttatctgttcacttgtgccctgactttcaactctgtctccttcctcttcctacagtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcaggaccagaccagctttcaaaaagaaaattgttaaagagagcatgaaaatggttctatgactttgcctgatacagatgctacttgacttacgatggtgttacttcctgataaactcgtcgtaagttgaaaatattatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 12 - protein, 214 aa, Homo sapiens
MFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQMLLDLRWCYFLINSS

SEQ ID NO: 13 - DNA, 2331 bp, Homo sapiens
tgaggccaggagatggaggctgcagtgagctgtgatcacaccactgtgctccagcctgagtgacagagcaagaccctatctcaaaaaaaaaaaaaaaaaagaaaagctcctgaggtgtagacgccaactctctctagctcgctagtgggttgcaggaggtgcttacgcatgtttgtttctttgctgccgtcttccagttgctttatctgttcacttgtgccctgactttcaactctgtctccttcctcttcctacagtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatgctacttgacttacgatggtgttacttcctgataaactcgtcgtaagttgaaaatattatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 14 - protein, 354 aa, Homo sapiens
MDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 15 - DNA, 2708 bp, Homo sapiens
gatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactgtgagtggatccattggaagggcaggcccaccacccccaccccaaccccagccccctagcagagacctgtgggaagcgaaaattccatgggactgactttctgctcttgtctttcagacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 16 - DNA, 2591 bp, Homo sapiens; identical to SEQ ID NO: 2

SEQ ID NO: 17 - DNA, 2588 bp, Homo sapiens; identical to SEQ ID NO: 3

SEQ ID NO: 18 - protein, 307 aa, Homo sapiens
MDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQMLLDLRWCYFLINSS

SEQ ID NO: 19 - DNA, 2651 bp, Homo sapiens; identical to SEQ ID NO: 7

SEQ ID NO: 20 - protein, 302 aa, Homo sapiens
MDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC

SEQ ID NO: 21 - DNA, 2724 bp, Homo sapiens; identical to SEQ ID NO: 5

SEQ ID NO: 22 - protein, 234 aa, Homo sapiens
MAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 23 - DNA, 2271 bp, Homo sapiens; identical to SEQ ID NO: 9

SEQ ID NO: 24 - protein, 182 aa, Homo sapiens
MAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC

SEQ ID NO: 25 - DNA, 2404 bp, Homo sapiens; identical to SEQ ID NO: 11

SEQ ID NO: 26 - protein, 187 aa, Homo sapiens
MAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQMLLDLRWCYFLINSS

SEQ ID NO: 27 - DNA, 2331 bp, Homo sapiens; identical to SEQ ID NO: 13

SEQ ID NO: 28 - protein, 381 aa, Mus musculus
MTAMEESQSDISLELPLSQETFSGLWKLLPPEDILPSPHCMDDLLLPQDVEEFFEGPSEALRVSGAPAAQDPVTETPGPVAPAPATPWPLSSFVPSQKTYQGNYGFHLGFLQSGTAKSVMCTYSPPLNKLFCQLAKTCPVQLWVSATPPAGSRVRAMAIYKKSQHMTEVVRRCPHHERCSDGDGLAPPQHLIRVEGNLYPEYLEDRQTFRHSVVVPYEPPEAGSEYTTIHYKYMCNSSCMGGMNRRPILTIITLEDSSGNLLGRDSFEVRVCACPGRDRRTEEENFRKKEVLCPELPPGSAKRALPTCTSASPPQKKKPLDGEYFTLKIRGRKRFEMFRELNEALELKDAHATEESGDSRAHSSLQPRAFQALIKEESPNC

SEQ ID NO: 29 - DNA, 1877 bp, Mus musculus
tttcccctcccacgtgctcaccctggctaaagttctgtagcttcagttcattgggaccatcctggctgtaggtagcgactacagttagggggcacctagcattcaggccctcatcctcctccttcccagcagggtgtcacgcttctccgaagactggatgactgccatggaggagtcacagtcggatatcagcctcgagctccctctgagccaggagacattttcaggcttatggaaactacttcctccagaagatatcctgccatcacctcactgcatggacgatctgttgctgccccaggatgttgaggagttttttgaaggcccaagtgaagccctccgagtgtcaggagctcctgcagcacaggaccctgtcaccgagacccctgggccagtggcccctgccccagccactccatggcccctgtcatcttttgtcccttctcaaaaaacttaccagggcaactatggcttccacctgggcttcctgcagtctgggacagccaagtctgttatgtgcacgtactctcctcccctcaataagctattctgccagctggcgaagacgtgccctgtgcagttgtgggtcagcgccacacctccagctgggagccgtgtccgcgccatggccatctacaagaagtcacagcacatgacggaggtcgtgagacgctgcccccaccatgagcgctgctccgatggtgatggcctggctcctccccagcatcttatccgggtggaaggaaatttgtatcccgagtatctggaagacaggcagacttttcgccacagcgtggtggtaccttatgagccacccgaggccggctctgagtataccaccatccactacaagtacatgtgtaatagctcctgcatggggggcatgaaccgccgacctatccttaccatcatcacactggaagactccagtgggaaccttctgggacgggacagctttgaggttcgtgtttgtgcctgccctgggagagaccgccgtacagaagaagaaaatttccgcaaaaaggaagtcctttgccctgaactgcccccagggagcgcaaagagagcgctgcccacctgcacaagcgcctctcccccgcaaaagaaaaaaccacttgatggagagtatttcaccctcaagatccgcgggcgtaaacgcttcgagatgttccgggagctgaatgaggccttagagttaaaggatgcccatgctacagaggagtctggagacagcagggctcactccagcctccagcctagagccttccaagccttgatcaaggaggaaagcccaaactgctagctcccatcacttcatccctccccttttctgtcttcctatagctacctgaagaccaagaagggccagtctacttcccgccataaaaaaacaatggtcaagaaagtggggcctgactcagactgactgcctctgcatcccgtccccatcaccagcctccccctctccttgctgtcttatgacttcagggctgagacacaatcctcccggtcccttctgctgccttttttaccttgtagctagggctcagccccctctctgagtagtggttcctggcccaagttggggaataggttgatagttgtcaggtctctgctggcccagcgaaattctatccagccagttgttggaccctggcacctacaatgaaatctcaccctaccccacaccctgtaagattctatcttgggccctcatagggtccatatcctccagggcctactttccttccattctgcaaagcctgtctgcatttatccaccccccaccctgtctccctcttttttttttttttacccctttttatatatcaatttcctattttacaataaaattttgttatcacttaaaaaaaaaa

SEQ ID NO: 30 - protein, 390 aa, Mus musculus
MTAMEESQSDISLELPLSQETFSGLWKLLPPEDILPSPHCMDDLLLPQDVEEFFEGPSEALRVSGAPAAQDPVTETPGPVAPAPATPWPLSSFVPSQKTYQGNYGFHLGFLQSGTAKSVMCTYSPPLNKLFCQLAKTCPVQLWVSATPPAGSRVRAMAIYKKSQHMTEVVRRCPHHERCSDGDGLAPPQHLIRVEGNLYPEYLEDRQTFRHSVVVPYEPPEAGSEYTTIHYKYMCNSSCMGGMNRRPILTIITLEDSSGNLLGRDSFEVRVCACPGRDRRTEEENFRKKEVLCPELPPGSAKRALPTCTSASPPQKKKPLDGEYFTLKIRGRKRFEMFRELNEALELKDAHATEESGDSRAHSSYLKTKKGQSTSRHKKTMVKKVGPDSD

SEQ ID NO: 31 - DNA, 1781 bp, Mus musculus
tttcccctcccacgtgctcaccctggctaaagttctgtagcttcagttcattgggaccatcctggctgtaggtagcgactacagttagggggcacctagcattcaggccctcatcctcctccttcccagcagggtgtcacgcttctccgaagactggatgactgccatggaggagtcacagtcggatatcagcctcgagctccctctgagccaggagacattttcaggcttatggaaactacttcctccagaagatatcctgccatcacctcactgcatggacgatctgttgctgccccaggatgttgaggagttttttgaaggcccaagtgaagccctccgagtgtcaggagctcctgcagcacaggaccctgtcaccgagacccctgggccagtggcccctgccccagccactccatggcccctgtcatcttttgtcccttctcaaaaaacttaccagggcaactatggcttccacctgggcttcctgcagtctgggacagccaagtctgttatgtgcacgtactctcctcccctcaataagctattctgccagctggcgaagacgtgccctgtgcagttgtgggtcagcgccacacctccagctgggagccgtgtccgcgccatggccatctacaagaagtcacagcacatgacggaggtcgtgagacgctgcccccaccatgagcgctgctccgatggtgatggcctggctcctccccagcatcttatccgggtggaaggaaatttgtatcccgagtatctggaagacaggcagacttttcgccacagcgtggtggtaccttatgagccacccgaggccggctctgagtataccaccatccactacaagtacatgtgtaatagctcctgcatggggggcatgaaccgccgacctatccttaccatcatcacactggaagactccagtgggaaccttctgggacgggacagctttgaggttcgtgtttgtgcctgccctgggagagaccgccgtacagaagaagaaaatttccgcaaaaaggaagtcctttgccctgaactgcccccagggagcgcaaagagagcgctgcccacctgcacaagcgcctctcccccgcaaaagaaaaaaccacttgatggagagtatttcaccctcaagatccgcgggcgtaaacgcttcgagatgttccgggagctgaatgaggccttagagttaaaggatgcccatgctacagaggagtctggagacagcagggctcactccagctacctgaagaccaagaagggccagtctacttcccgccataaaaaaacaatggtcaagaaagtggggcctgactcagactgactgcctctgcatcccgtccccatcaccagcctccccctctccttgctgtcttatgacttcagggctgagacacaatcctcccggtcccttctgctgccttttttaccttgtagctagggctcagccccctctctgagtagtggttcctggcccaagttggggaataggttgatagttgtcaggtctctgctggcccagcgaaattctatccagccagttgttggaccctggcacctacaatgaaatctcaccctaccccacaccctgtaagattctatcttgggccctcatagggtccatatcctccagggcctactttccttccattctgcaaagcctgtctgcatttatccaccccccaccctgtctccctcttttttttttttttacccctttttatatatcaatttcctattttacaataaaattttgttatcacttaaaaaaaaaa

SEQ ID NO: 32 - protein, 928 aa, Homo sapiens
MPPKTPRKTAATAAAAAAEPPAPPPPPPPEEDPEQDSGPEDLPLVRLEFEETEEPDFTALCQKLKIPDHVRERAWLTWEKVSSVDGVLGGYIQKKKELWGICIFIAAVDLDEMSFTFTELQKNIEISVHKFFNLLKEIDTSTKVDNAMSRLLKKYDVLFALFSKLERTCELIYLTQPSSSISTEINSALVLKVSWITFLLAKGEVLQMEDDLVISFQLMLCVLDYFIKLSPPMLLKEPYKTAVIPINGSPRTPRRGQNRSARIAKQLENDTRIIEVLCKEHECNIDEVKNVYFKNFIPFMNSLGLVTSNGLPEVENLSKRYEEIYLKNKDLDARLFLDHDKTLQTDSIDSFETQRTPRKSNLDEEVNVIPPHTPVRTVMNTIQQLMMILNSASDQPSENLISYFNNCTVNPKESILKRVKDIGYIFKEKFAKAVGQGCVEIGSQRYKLGVRLYYRVMESMLKSEEERLSIQNFSKLLNDNIFHMSLLACALEVVMATYSRSTSQNLDSGTDLSFPWILNVLNLKAFDFYKVIESFIKAEGNLTREMIKHLERCEHRIMESLAWLSDSPLFDLIKQSKDREGPTDHLESACPLNLPLQNNHTAADMYLSPVRSPKKKGSTTRVNSTANAETQATSAFQTQKPLKSTSLSLFYKKVYRLAYLRLNTLCERLLSEHPELEHIIWTLFQHTLQNEYELMRDRHLDQIMMCSMYGICKVKNIDLKFKIIVTAYKDLPHAVQETFKRVLIKEEEYDSIIVFYNSVFMQRLKTNILQYASTRPPTLSPIPHIPRSPYKFPSSPLRIPGGNIYISPLKSPYKISEGLPTPTKMTPRSRILVSIGESFGTSEKFQKINQMVCNSDRVLKRSAEGSNPPKPLKKLRFDIEGSDEADGSKHLPGESKFQQKLAEMTSTRTRMQKQKMNDSMDTSNKEEK

SEQ ID NO: 33 - DNA, 4772 bp, Homo sapiens
gctcagttgccgggcgggggagggcgcgtccggtttttctcaggggacgttgaaattatttttgtaacgggagtcgggagaggacggggcgtgccccgacgtgcgcgcgcgtcgtcctccccggcgctcctccacagctcgctggctcccgccgcggaaaggcgtcatgccgcccaaaaccccccgaaaaacggccgccaccgccgccgctgccgccgcggaacccccggcaccgccgccgccgccccctcctgaggaggacccagagcaggacagcggcccggaggacctgcctctcgtcaggcttgagtttgaagaaacagaagaacctgattttactgcattatgtcagaaattaaagataccagatcatgtcagagagagagcttggttaacttgggagaaagtttcatctgtggatggagtattgggaggttatattcaaaagaaaaaggaactgtggggaatctgtatctttattgcagcagttgacctagatgagatgtcgttcacttttactgagctacagaaaaacatagaaatcagtgtccataaattctttaacttactaaaagaaattgataccagtaccaaagttgataatgctatgtcaagactgttgaagaagtatgatgtattgtttgcactcttcagcaaattggaaaggacatgtgaacttatatatttgacacaacccagcagttcgatatctactgaaataaattctgcattggtgctaaaagtttcttggatcacatttttattagctaaaggggaagtattacaaatggaagatgatctggtgatttcatttcagttaatgctatgtgtccttgactattttattaaactctcacctcccatgttgctcaaagaaccatataaaacagctgttatacccattaatggttcacctcgaacacccaggcgaggtcagaacaggagtgcacggatagcaaaacaactagaaaatgatacaagaattattgaagttctctgtaaagaacatgaatgtaatatagatgaggtgaaaaatgtttatttcaaaaattttataccttttatgaattctcttggacttgtaacatctaatggacttccagaggttgaaaatctttctaaacgatacgaagaaatttatcttaaaaataaagatctagatgcaagattatttttggatcatgataaaactcttcagactgattctatagacagttttgaaacacagagaacaccacgaaaaagtaaccttgatgaagaggtgaatgtaattcctccacacactccagttaggactgttatgaacactatccaacaattaatgatgattttaaattcagcaagtgatcaaccttcagaaaatctgatttcctattttaacaactgcacagtgaatccaaaagaaagtatactgaaaagagtgaaggatataggatacatctttaaagagaaatttgctaaagctgtgggacagggttgtgtcgaaattggatcacagcgatacaaacttggagttcgcttgtattaccgagtaatggaatccatgcttaaatcagaagaagaacgattatccattcaaaattttagcaaacttctgaatgacaacatttttcatatgtctttattggcgtgcgctcttgaggttgtaatggccacatatagcagaagtacatctcagaatcttgattctggaacagatttgtctttcccatggattctgaatgtgcttaatttaaaagcctttgatttttacaaagtgatcgaaagttttatcaaagcagaaggcaacttgacaagagaaatgataaaacatttagaacgatgtgaacatcgaatcatggaatcccttgcatggctctcagattcacctttatttgatcttattaaacaatcaaaggaccgagaaggaccaactgatcaccttgaatctgcttgtcctcttaatcttcctctccagaataatcacactgcagcagatatgtatctttctcctgtaagatctccaaagaaaaaaggttcaactacgcgtgtaaattctactgcaaatgcagagacacaagcaacctcagccttccagacccagaagccattgaaatctacctctctttcactgttttataaaaaagtgtatcggctagcctatctccggctaaatacactttgtgaacgccttctgtctgagcacccagaattagaacatatcatctggacccttttccagcacaccctgcagaatgagtatgaactcatgagagacaggcatttggaccaaattatgatgtgttccatgtatggcatatgcaaagtgaagaatatagaccttaaattcaaaatcattgtaacagcatacaaggatcttcctcatgctgttcaggagacattcaaacgtgttttgatcaaagaagaggagtatgattctattatagtattctataactcggtcttcatgcagagactgaaaacaaatattttgcagtatgcttccaccaggccccctaccttgtcaccaatacctcacattcctcgaagcccttacaagtttcctagttcacccttacggattcctggagggaacatctatatttcacccctgaagagtccatataaaatttcagaaggtctgccaacaccaacaaaaatgactccaagatcaagaatcttagtatcaattggtgaatcattcgggacttctgagaagttccagaaaataaatcagatggtatgtaacagcgaccgtgtgctcaaaagaagtgctgaaggaagcaaccctcctaaaccactgaaaaaactacgctttgatattgaaggatcagatgaagcagatggaagtaaacatctcccaggagagtccaaatttcagcagaaactggcagaaatgacttctactcgaacacgaatgcaaaagcagaaaatgaatgatagcatggatacctcaaacaaggaagagaaatgaggatctcaggaccttggtggacactgtgtacacctctggattcattgtctctcacagatgtgactgtataactttcccaggttctgtttatggccacatttaatatcttcagctctttttgtggatataaaatgtgcagatgcaattgtttgggtgattcctaagccacttgaaatgttagtcattgttatttatacaagattgaaaatcttgtgtaaatcctgccatttaaaaagttgtagcagattgtttcctcttccaaagtaaaattgctgtgctttatggatagtaagaatggccctagagtgggagtcctgataacccaggcctgtctgactactttgccttcttttgtagcatataggtgatgtttgctcttgtttttattaatttatatgtatatttttttaatttaacatgaacacccttagaaaatgtgtcctatctatcttccaaatgcaatttgattgactgcccattcaccaaaattatcctgaactcttctgcaaaaatggatattattagaaattagaaaaaaattactaattttacacattagattttattttactattggaatctgatatactgtgtgcttgttttataaaattttgcttttaattaaataaaagctggaagcaaagtataaccatatgatactatcatactactgaaacagatttcatacctcagaatgtaaaagaacttactgattattttcttcatccaacttatgtttttaaatgaggattattgatagtactcttggtttttataccattcagatcactgaatttataaagtacccatctagtacttgaaaaagtaaagtgttctgccagatcttaggtatagaggaccctaacacagtatatcccaagtgcactttctaatgtttctgggtcctgaagaattaagatacaaattaattttactccataaacagactgttaattataggagccttaatttttttttcatagagatttgtctaattgcatctcaaaattattctgccctccttaatttgggaaggtttgtgttttctctggaatggtacatgtcttccatgtatcttttgaactggcaattgtctatttatcttttatttttttaagtcagtatggtctaacactggcatgttcaaagccacattatttctagtccaaaattacaagtaatcaagggtcattatgggttaggcattaatgtttctatctgattttgtgcaaaagcttcaaattaaaacagctgcattagaaaaagaggcgcttctcccctcccctacacctaaaggtgtatttaaactatcttgtgtgattaacttatttagagatgctgtaacttaaaataggggatatttaaggtagcttcagctagcttttaggaaaatcactttgtctaactcagaattatttttaaaaagaaatctggtcttgttagaaaacaaaattttattttgtgctcatttaagtttcaaacttactattttgacagttattttgataacaatgacactagaaaacttgactccatttcatcattgtttctgcatgaatatcatacaaatcagttagtttttaggtcaagggcttactatttctgggtcttttgctactaagttcacattagaattagtgccagaattttaggaacttcagagatcgtgtattgagatttcttaaataatgcttcagatattattgctttattgcttttttgtattggttaaaactgtacatttaaaattgctatgttactattttctacaattaatagtttgtctattttaaaataaattagttgttaagagtcttaa

SEQ ID NO: 34 - protein, 921 aa, Mus musculus
MPPKAPRRAAAAEPPPPPPPPPREDDPAQDSGPEELPLARLEFEEIEEPEFIALCQKLKVPDHVRERAWLTWEKVSSVDGILEGYIQKKKELWGICIFIAAVDLDEMPFTFTELQKSIETSVYKFFDLLKEIDTSTKVDNAMSRLLKKYNVLCALYSKLERTCELIYLTQPSSALSTEINSMLVLKISWITFLLAKGEVLQMEDDLVISFQLMLCVVDYFIKFSPPALLREPYKTAAIPINGSPRTPRRGQNRSARIAKQLENDTRIIEVLCKEHECNIDEVKNVYFKNFIPFINSLGIVSSNGLPEVESLSKRYEEVYLKNKDLDARLFLDHDKTLQTDPIDSFETERTPRKNNPDEEANVVTPHTPVRTVMNTIQQLMVILNSASDQPSENLISYFNNCTVNPKENILKRVKDVGHIFKEKFANAVGQGCVDIGVQRYKLGVRLYYRVMESMLKSEEERLSIQNFSKLLNDNIFHMSLLACALEVVMATYSRSTLQHLDSGTDLSFPWILNVLNLKAFDFYKVIESFIKVEANLTREMIKHLERCEHRIMESLAWLSDSPLFDLIKQSKDGEGPDNLEPACPLSLPLQGNHTAADMYLSPLRSPKKRTSTTRVNSAANTETQAASAFHTQKPLKSTSLALFYKKVYRLAYLRLNTLCARLLSDHPELEHIIWTLFQHTLQNEYELMRDRHLDQIMMCSMYGICKVKNIDLKFKIIVTAYKDLPHAAQETFKRVLIREEEFDSIIVFYNSVFMQRLKTNILQYASTRPPTLSPIPHIPRSPYKFSSSPLRIPGGNIYISPLKSPYKISEGLPTPTKMTPRSRILVSIGESFGTSEKFQKINQMVCNSDRVLKRSAEGGNPPKPLKKLRFDIEGADEADGSKHLPAESKFQQKLAEMTSTRTRMQKQRMNESKDVSNKEEK

SEQ ID NO: 35 - DNA, 4673 bp, Mus musculus
gttttcccgcggttggcccgggcgctcggttgccgggcgcgtccggttttcctcgggggacgttcccattatttttgtaacgggagtcgggtgaggacggggcgtgcccggcgtgcgcgcgcgcccgcccgcctccccgcgcgcctccctcggctgctcgcgccggcccgcgctgcgcgtcatgccgcccaaagccccgcgcagagccgcggccgccgagcccccgccaccgccgccgccgccgcctcgggaggacgaccccgcgcaggacagcggccccgaagagctgcccctggccaggcttgagtttgaagaaattgaagaacccgaatttattgcattatgtcaaaagttaaaggtacccgatcatgtcagagaaagagcttggctaacttgggagaaagtttcatccgtggatggaatcctggaaggatatattcaaaagaagaaggaactctggggcatctgcatctttatcgcagcagttgatctagatgagatgccattcacttttactgagctacagaaaagcatagaaaccagtgtctataaattctttgacttattaaaagaaatcgataccagtaccaaggttgataatgctatgtcaagactattgaagaagtataatgtgttatgtgcactctacagcaaattagaacggacgtgtgaacttatatatttgacacaacccagcagtgcgttatctactgaaataaattctatgttggtgctaaaaatttcttggatcacttttttactagctaaaggagaagtattacagatggaagatgacctggtaatctcatttcagctaatgttgtgtgtagttgactattttattaagttctcacctcctgcactactcagagagccatacaaaacagctgcaatccccattaatggttcacctcgaacacccagaagaggtcagaacaggagcgctcggatagcaaaacaactagaaaatgatacgaggattatcgaggttctctgtaaagaacacgagtgtaatatagatgaggtgaaaaatgtttatttcaaaaattttatcccttttataaattcacttggaattgtatcatctaatggacttccagaggttgaaagtctttctaaacgctatgaagaagtttatcttaaaaacaaagatttagatgcaagactgtttttggatcatgataaaacacttcagactgatcctatagacagttttgaaacagagagaacgccacgaaaaaacaaccctgatgaagaggcaaacgtggttactccacacactccagttaggactgttatgaatactattcaacaattaatggtgattttaaattctgcaagtgatcagccatcagaaaatctgatttcctacttcaataattgcacagtgaatccaaaagaaaatatcctaaagagagtaaaggatgttgggcacatctttaaagagaagtttgctaacgctgtgggccagggctgtgttgacatcggagtacagcgatataaacttggagtccgattgtattaccgtgtgatggaatccatgcttaaatcagaagaagaacgtttgtccattcagaattttagcaaactcctaaatgacaacatctttcatatgtctttactggcctgtgctcttgaagttgtaatggctacgtatagcagaagtacattgcagcatcttgattctggaacagatttgtccttcccgtggattctgaacgtacttaatttaaaagcctttgatttttacaaagtgattgaaagttttatcaaagtggaagccaacttgacaagagaaatgataaaacatttagaaagatgtgagcatcgaatcatggaatcccttgcatggctttcagattcacctttatttgatctcattaagcagtccaaggatggagaaggacctgataaccttgaacctgcttgtcctctcagcctgcctctccagggtaaccatactgcagcagatatgtatctttctcctctaagatctccaaagaaaagaacttccactacacgtgtaaattctgctgcaaatacagagacacaagcagcctcagccttccatactcagaagccattgaaatctacctcccttgccctgttttacaaaaaagtgtaccgtctagcatatctccgactaaatacactctgtgcacgccttctgtctgaccacccagagctagagcacatcatctggactctgtttcagcatacattgcaaaatgagtatgagctcatgagagaccgacatttggaccagattatgatgtgctctatgtatggcatctgcaaggtgaagaacatcgacctcaagttcaaaatcatcgtcactgcctacaaggatcttcctcacgctgcccaggagacctttaaacgtgttttgatcagagaagaggagtttgattccattatagtattctataactccgttttcatgcagagactaaaaacaaatattttacagtatgcctccaccaggcctcctaccttgtcaccaatacctcacattcctcgaagcccttacaagttttctagttcacccttacggattcctggaggtaacatctatatatcacccctaaagagtccttataaaatttcagaaggtctgccaacacccacaaaaatgactccgagatcaagaatcttggtctcaattggtgaatcatttgggacatctgaaaagttccagaaaataaaccagatggtgtgtaatagtgacagagtgctcaaaagaagtgctgaaggcggcaacccccccaaaccactgaaaaagctgcgctttgacatcgagggagccgatgaagcagatgggagtaaacatctcccagcggagtccaaattccaacagaaactggcagaaatgacttccactcgaacacgaatgcaaaagcagagaatgaatgagagcaaggatgtctcaaacaaggaggaaaagtgaggacctcagggccctggaccctcagccctggggacaccagactcctggctcatggttgtgactagttcccaggttctgctcatgttagagatataaaatgtgcaggtacaagctgaatatttgtgtgggtgattcgtaagccacttcaatgttgtaatcaatggtagagaattgatagcacacaggattgagaatcttgtgcagatcatgccatttaaaatatgaaagcaagttgtttgcatttccaacatgagcctgctgccctcagaagtgagggcctctgcaggggtcctgaccccctacatctggctgactactttgcctttcctcatggcacatgtgtgatgtttgctcttggttttattaatttatatgtattttttaatttaacgtgaatacccttagaaaatgtgtcttgtcttccaaatgcaagttgattgactgtccacattcaaccaaattatcttgaatcttctactgaaacagattgttacaaactgggaaaaagtactaatttctacacattggactattttaatattagaatctaatgtgccgtgtgtttgtttcacatattttacttttgaaatacaagcaaaaaaagtataatcatatgatactgtcttactactgacacagatttcatacctcagaccctctaagaaccgattcttttattcacccaacacatgctttgaactgaagactattgataatactccgaggttgtttttttctttcaatcattcagatcactgaatttataagtacccatgtagtacttgaaagtcaagtttggccacaactgtgcttaagaggaccctagtacagtacaacccaagtgcactttttaatgtttctgggtcctgaagaatcaagatacaaattaattgtgatttacaagcagactgttaactatagaagccttcagtttttttccctcatagacgtgtctaattacatctcaacagtttactctgttcttctacatctggggatgtttgtgttctctctggaatggtacatcttccagggtcttttgaacttgcagttatctattttttaagccaatctggtctaataactctggcttcttcaaagccacaccatttctagtccagctgtgcagaaacttcagatgaaaacagctgcattgaaaatagaggcactcccttcaccccccacctaaaggtgtatttaaattatcttgtgggattaacttatttagagatggtataatttaaaataggggatatttaaggtagcatcagctagcatttaagaaaatcactttttctaaactccatactttttgaaaagaaatctggtcttgttaggaaacaaatttctattttgtcctcaatttagtttcagttttactagtttgatagttatctcaataacaaaagcaatagacagcttcccccatttcttcattaagttttgcatgatcatcacacagattagttaggttttaggtcaagggcttaccatacttctaggtcttttgctagtgagttcaagttagaattagtgacagaatcataggaattttcagagatcctgcttcgagatttcttaaagctgcagacactgcactattggttttgtttttttgtaccggttgaaactatacattcaaattgctatgttcctattttctataatagtttgtctattttaaaataaactagttgttcagagccttaaaaaaaaaaaaaaaaaa

SEQ ID NO: 36 - protein, 956 aa, Homo sapiens
MSSGLWSQEKVTSPYWEERIFYLLLQECSVTDKQTQKLLKVPKGSIGQYIQDRSVGHSRIPSAKGKKNQIGLKILEQPHAVLFVDEKDVVEINEKFTELLLAITNCEERFSLFKNRNRLSKGLQIDVGCPVKVQLRSGEEKFPGVVRFRGPLLAERTVSGIFFGVELLEEGRGQGFTDGVYQGKQLFQCDEDCGVFVALDKLELIEDDDTALESDYAGPGDTMQVELPPLEINSRVSLKVGETIESGTVIFCDVLPGKESLGYFVGVDMDNPIGNWDGRFDGVQLCSFACVESTILLHINDIIPALSESVTQERRPPKLAFMSRGVGDKGSSSHNKPKATGSTSDPGNRNRSELFYTLNGSSVDSQPQSKSKNTWYIDEVAEDPAKSLTEISTDFDRSSPPLQPPPVNSLTTENRFHSLPFSLTKMPNTNGSIGHSPLSLSAQSVMEELNTAPVQESPPLAMPPGNSHGLEVGSLAEVKENPPFYGVIRWIGQPPGLNEVLAGLELEDECAGCTDGTFRGTRYFTCALKKALFVKLKSCRPDSRFASLQPVSNQIERCNSLAFGGYLSEVVEENTPPKMEKEGLEIMIGKKKGIQGHYNSCYLDSTLFCLFAFSSVLDTVLLRPKEKNDVEYYSETQELLRTEIVNPLRIYGYVCATKIMKLRKILEKVEAASGFTSEEKDPEEFLNILFHHILRVEPLLKIRSAGQKVQDCYFYQIFMEKNEKVGVPTIQQLLEWSFINSNLKFAEAPSCLIIQMPRFGKDFKLFKKIFPSLELNITDLLEDTPRQCRICGGLAMYECRECYDDPDISAGKIKQFCKTCNTQVHLHPKRLNHKYNPVSLPKDLPDWDWRHGCIPCQNMELFAVLCIETSHYVAFVKYGKDDSAWLFFDSMADRDGGQNGFNIPQVTPCPEVGEYLKMSLEDLHSLDSRRIQGCARRLLCDAYMCMYQSPTMSLYK

SEQ ID NO: 37 - DNA, 8730 bp, Homo sapiens
gtgcggttcggaggcggggcaggtgggggcgggcccaggtagcaggtttggctgcgcgggggccgcgcgtcggagtttccccctttctagggtgaggatggttctacacagccacccggagttccttagttgaaaggtgcgccctgctgtgacagaatgtggtaattgtaatctttaacattttcatgtaaaacatatttcctgatcatctttccattgtcttcatggaaaattgataaatatttgtgccttccaactctcgtcttggttgaatgacttcatcttaatacaacatggacaccacgttgctgaaaacatgctttgggactgccactgaatttatcttttgcggttttatgacaaagttattagtagtttcccttttttgaattagtattttgaagttaatatcacaatgagttcaggcttatggagccaagaaaaagtcacttcaccctactgggaagagcggattttttacttgcttcttcaagaatgcagcgttacagacaaacaaacacaaaagctccttaaagtaccgaagggaagtataggacagtatattcaagatcgttctgtggggcattcaaggattccttctgcaaaaggcaagaaaaatcagattggattaaaaattctagagcaacctcatgcagttctctttgttgatgaaaaggatgttgtagagataaatgaaaagttcacagagttacttttggcaattaccaattgtgaggagaggttcagcctgtttaaaaacagaaacagactaagtaaaggcctccaaatagacgtgggctgtcctgtgaaagtacagctgagatctggggaagaaaaatttcctggagttgtacgcttcagaggacccctgttagcagagaggacagtctccggaatattctttggagttgaattgctggaagaaggtcgtggtcaaggtttcactgacggggtgtaccaagggaaacagctttttcagtgtgatgaagattgtggcgtgtttgttgcattggacaagctagaactcatagaagatgatgacactgcattggaaagtgattacgcaggtcctggggacacaatgcaggtcgaacttcctcctttggaaataaactccagagtttctttgaaggttggagaaacaatagaatctggaacagttatattctgtgatgttttgccaggaaaagaaagcttaggatattttgttggtgtggacatggataaccctattggcaactgggatggaagatttgatggagtgcagctttgtagttttgcgtgtgttgaaagtacaattctattgcacatcaatgatatcatcccagctttatcagagagtgtgacgcaggaaaggaggcctcccaaacttgcctttatgtcaagaggtgttggggacaaaggttcatccagtcataataaaccaaaggctacaggatctacctcagaccctggaaatagaaacagatctgaattattttataccttaaatgggtcttctgttgactcacaaccacaatccaaatcaaaaaatacatggtacattgatgaagttgcagaagaccctgcaaaatctcttacagagatatctacagactttgaccgttcttcaccaccactccagcctcctcctgtgaactcactgaccaccgagaacagattccactctttaccattcagtctcaccaagatgcccaataccaatggaagtattggccacagtccactttctctgtcagcccagtctgtaatggaagagctaaacactgcacccgtccaagagagtccacccttggccatgcctcctgggaactcacatggtctagaagtgggctcattggctgaagttaaggagaaccctcctttctatggggtaatccgttggatcggtcagccaccaggactgaatgaagtgctcgctggactggaactggaagatgagtgtgcaggctgtacggatggaaccttcagaggcactcggtatttcacctgtgccctgaagaaggcgctgtttgtgaaactgaagagctgcaggcctgactctaggtttgcatcattgcagccggtttccaatcagattgagcgctgtaactctttagcatttggaggctacttaagtgaagtagtagaagaaaatactccaccaaaaatggaaaaagaaggcttggagataatgattgggaagaagaaaggcatccagggtcattacaattcttgttacttagactcaaccttattctgcttatttgcttttagttctgttctggacactgtgttacttagacccaaagaaaagaacgatgtagaatattatagtgaaacccaagagctactgaggacagaaattgttaatcctctgagaatatatggatatgtgtgtgccacaaaaattatgaaactgaggaaaatacttgaaaaggtggaggctgcatcaggatttacctctgaagaaaaagatcctgaggaattcttgaatattctgtttcatcatattttaagggtagaacctttgctaaaaataagatcagcaggtcaaaaggtacaagattgttacttctatcaaatttttatggaaaaaaatgagaaagttggcgttcccacaattcagcagttgttagaatggtcttttatcaacagtaacctgaaatttgcagaggcaccatcatgtctgattattcagatgcctcgatttggaaaagactttaaactatttaaaaaaatttttccttctctggaattaaatataacagatttacttgaagacactcccagacagtgccggatatgtggagggcttgcaatgtatgagtgtagagaatgctacgacgatccggacatctcagctggaaaaatcaagcagttttgtaaaacctgcaacactcaagtccaccttcatccgaagaggctgaatcataaatataacccagtgtcacttcccaaagacttacccgactgggactggagacacggctgcatcccttgccagaatatggagttatttgctgttctctgcatagaaacaagccactatgttgcttttgtgaagtatgggaaggacgattctgcctggctcttctttgacagcatggccgatcgggatggtggtcagaatggcttcaacattcctcaagtcaccccatgcccagaagtaggagagtacttgaagatgtctctggaagacctgcattccttggactccaggagaatccaaggctgtgcacgaagactgctttgtgatgcatatatgtgcatgtaccagagtccaacaatgagtttgtacaaataactggggtcatcgggaaaggcaaagaaactgaaggcagagtcctaacgttgcatcttattcgagctggcagttctgttcacgtccattgccggcaatggatgtctttgtggtgatgatccttcagaaaaggatgcctctgtttaaaaacaaattgcttttgtgtccctgaagtatttaataagaagcattttgcactctagaaagtatgtttgtgttggttttttaagaagtctaaatgaagttattaatacctgaagctttaagttaagtgcattgatcatatgatatttttggaagcatacaattttaattgtggaagtttaaagcctcttttagtccattgagaatgtaaataaatgtgtcttctttatggaccaaggatatgaaatcatttttcttttgtagctaacggttgccttgaggaagaaataatttggttttattaagagtctactctcaatccagttattagagatgtactgagtttgatttgttaatcctttctatatactgctgatcttgcatgtctacaatctgctcagtttttctgtgtttctgcaatagtggtcagaaaaatacttaaattcccttaatggtgttgttttctatttgttctggttttgagataaatgagtgattctgtccccaaatgtccatttttgaagtgattttcctggaggattagggtatttagcagttgaagctcttcattcatagtagttactgtcagctaacaggttttttaaggcttttaactattaatattttatggaatggggcaaagtaaattgatgaaagaattggagtgataatagtcctttacaaacatacagtccataagaaaatgaatttggcatatagaattattacaatttcctgggagagatggatatttaaacctctattattttagacaagactgtctagaacttaagtttgatctgtcagccagtactcccattaaattcagtgtagtttcacttgatagaatcagatatgttatcgaaatgttagcagcagcttcatcctccttctgattaaagtaagtagaaatgggatgttttgtttaataacagccatagtgtgtgtttagaccacagcggatgttgtagaccaggaccatagatgatacatgtcagtgctgtggaatgtgcattctctgagtgttgttttgtggtatcattgtctttcctgaatgactttctaactgtgcagaaaggcagaaaagtcatcatatgtatatgtcatatgactttataaaatatttaatgtgacaaaaagtggaaagaatctttacaaaccctgcaattacttttttaaaggcacttttactctttggttttatcattccattttgctaatatttactagctttataaattacagtaaggtacaaaaactcatcttgtaatattttcatttttgaagtgaaaaagtacatatattttgcacaaggttttatactgctaagtgcttggttggggtggtgagatgatgattagatcaggggtgaggctgagagactctgggtttagggctagccctgcctccatctcccttgggtaaaatgaagggtgtggggtaaaagatgcataaggccttttctagctctgacagcctagaagtccaatcaccctgtaataaatatgtgttgaatgaagaaatgggtgaatgagcttgtcaatgtgattttaaaaaattgactacctggaggaatgattaggaatctaaatgaagccagccctcggtatctgcaggtttctcatccatggattcaaccaactgcaaatggaaaatacgattttttttaaaaaaaggatggttacatccgtattgaacatgtacagacttttttcttgtcattattctctgaacaatacaagaactctttatgtagcatttacatttattaggtattataagtaatctagagattatttaattaaaatatacaggaggatgtgtgtcagttatatgcaaattctgtaccattttgtatcagggaattgagcatcttcagatgttggtatctgcagggatcctggaaccaaacccctgcagatactaagggctgacgatctaggtaagactggatttaacagttggaaaaaaaaaaaaaaaaggagagagaagacagttcctttcctgtagaaattaaaacaaaatacaaattgaggaagctctgctacccaggctgtcatggtagagaacttgaagaagacctgtttggatggacacctggtttcaaaagtcaggtgtggagactgttaaatgggagggcctcatccataaatgatttctggcaacgtcttcttcaggtggagcttgacgtctttttaatgttacttggggagggagtgctcattaagggatgccagggccagctctggtggttcctggggaggctgcgtccttccctgcttctgcatgtcatgaggcagcaggaaggtttcccctgcacctgtctgtcctggctccctctgggtagccccctactgttctgtgcttcagcacagcctggtttgtcaagaggcacatagttggggctgggctgcatggcacaggggcttatgtgcctgctggttatttaattttcagccttaagttttctttaatattttcctgttggctatttaaaggttttggttatcttttattccttatctacaatcaagatgacaatgtaattgaattatcttatttataacacggttcgtgattcatgattcatgattacaagtagaaaatatgtcatgttcctcacctccaaataaatatgtgtgtgtctgtgtgtgtgtatatatgtatgtggcggagagggagagagtggggaaggagagcagtgttatcatacatagagaggctaaatgtgtcccatccctcactgtcagctttataaaggagtttgactccatccacagaagaatgttttataagactaggaaaacacgttgaaaactaggataaacagcaacaaaaatcaactaaatatgttgttactgttgctaaggattttctccttagaataatttaggatttttaaaaatttctgtttgccaaatgctgtagataaatggccagattcttcctatccctaggattcctttattattttttttcacagattttgagaacaagggggagagatagtatggaagattaagattccattaatcttatagaactgtgttgtcacccaaattcctgcttgtttgaacatggcatcttcatagattcaggattcactaccctctatagctggatcttgaaaattatctggccagataattttgcatctgcttggatgattgtagactgagatgtgagtggaggataaagtattagacttttgctgagtaactgccaaccaagaagtatttatcggacacttactaggtgcctaggattgtatcagagggaatatgaaatgtgtccctgccctacctagttttaacgacagaatatctattaaaggctacttagctgaagggtaagggtgacaggtctaggggaagctttgggaggtggtgtgctgtgacagaaaaagtggcagagtagggacgagagacctgcattctagccctgtttctgtcacttgctctgtacacttagacaacagcttgacctcttgagctttagtttcctcctctgcataatgagagggttagactactgaattgtatgggaaaaaaatacaaattcctgggtcctaggccatgcctgctgaatccgactgttcaggaagaggcctaggaaatctgtgagggaatccccaggggaatctcgtgaccagccaggtgtgaaatctgctaactggaagatctcaaagcttccttcactttttgtgattttgtggtcatgtaacgttactgtattattctacgtaaatgtgggtacttggatgtttatcatactgtttctctgtgtttacatactaatttgtgtaagaaatgcattttagtctgtgtacctcaacctgctgtttgtttcctagaggtgttagtagtctttaaatacaagtaagacttaagaggatatttgatgttatttacctggatattttcttccccttttatttatttagaggaaattgagattctaggagccaaaaaaatgaaaacaaaattctaaggcaaagttaaagaaaaaaattacattatttcttaccatttgctactttataatgaaaatttaaaaattatatgggaagatttttctctgggataacaaatccttgtcataaagtaagaggtctttttaaagtaggtaggctataaggcctgtaatttaaaataatactcctttctctagggtttggtgcaattctccattaatgaagataacatttgaattccccaaagcaggtgaggagtcggggaggagaaagcgatgttaaaatgaaaactcactgcaaaagaggaggcagaggaagaaggaatgtaaaccccttaaagcagatgtgtgtggggccttatgaagaccaggattctgcgggtgtcaggggattgcccctcttgacagagactagggttttagactgaggcttcctgcagggtgttcgcattgcccttctccgttccccttcagacctttctggggagaagaggtgggaggaggggagaaagactgttcatcttattctgaatcctggagcagctgaaggttttctcttgagtcaggatgcagtggtaatgcattaaccagcaagtgtggccaaggataatgaaaaagtgggaaaggaaggtcctcctcctccctgattgtagcatccagcagtctctgtagccaggttactcaagaaccacatttgatttcctggccctttgccttggcagtgatggcatttttatttcactgtgttttaaagtcttcatttatttttataacatgggttagggagaagggccacaaatggagggattgtcctttcaagcaccacagcttcagataaaattagtactttcaaatattgtccactttaacttaaaaaattctagagggattatattggagactcaactgcccttggttttagtttataaaatggcctagtactgtggaattttaattttagaaagtcttagcatcagatcataaacattcattaaaagaactcacatcccatctgaaacttcccaggggagttgggattcttagtagattggtagaaaggggctcattttctactgcatttcccatttttggtatcttgttcagcatgttttatttttatttcttgtctgcagaacatcctatatttatgagaacattctttaagaagaccaccacatagaataccccttcctatcagctcgctctgatttagccttaattttgttaaattttttagagatgaatgaagtgctgctgtggaaagaaatgtacatatactatttctgtatcattaaaattacatttttatggttcaaaaaaaaaaaaaaaaa

SEQ ID NO: 38 - protein, 953 aa, Homo sapiens
MSSGLWSQEKVTSPYWEERIFYLLLQECSVTDKQTQKLLKVPKGSIGQYIQDRSVGHSRIPSAKGKKNQIGLKILEQPHAVLFVDEKDVVEINEKFTELLLAITNCEERFSLFKNRNRLSKGLQIDVGCPVKVQLRSGEEKFPGVVRFRGPLLAERTVSGIFFGVELLEEGRGQGFTDGVYQGKQLFQCDEDCGVFVALDKLELIEDDDTALESDYAGPGDTMQVELPPLEINSRVSLKVGETIESGTVIFCDVLPGKESLGYFVGVDMDNPIGNWDGRFDGVQLCSFACVESTILLHINDIIPESVTQERRPPKLAFMSRGVGDKGSSSHNKPKATGSTSDPGNRNRSELFYTLNGSSVDSQPQSKSKNTWYIDEVAEDPAKSLTEISTDFDRSSPPLQPPPVNSLTTENRFHSLPFSLTKMPNTNGSIGHSPLSLSAQSVMEELNTAPVQESPPLAMPPGNSHGLEVGSLAEVKENPPFYGVIRWIGQPPGLNEVLAGLELEDECAGCTDGTFRGTRYFTCALKKALFVKLKSCRPDSRFASLQPVSNQIERCNSLAFGGYLSEVVEENTPPKMEKEGLEIMIGKKKGIQGHYNSCYLDSTLFCLFAFSSVLDTVLLRPKEKNDVEYYSETQELLRTEIVNPLRIYGYVCATKIMKLRKILEKVEAASGFTSEEKDPEEFLNILFHHILRVEPLLKIRSAGQKVQDCYFYQIFMEKNEKVGVPTIQQLLEWSFINSNLKFAEAPSCLIIQMPRFGKDFKLFKKIFPSLELNITDLLEDTPRQCRICGGLAMYECRECYDDPDISAGKIKQFCKTCNTQVHLHPKRLNHKYNPVSLPKDLPDWDWRHGCIPCQNMELFAVLCIETSHYVAFVKYGKDDSAWLFFDSMADRDGGQNGFNIPQVTPCPEVGEYLKMSLEDLHSLDSRRIQGCARRLLCDAYMCMYQSPTMSLYK

SEQ ID NO: 39 - DNA, 8531 bp, Homo sapiens
aggtagcaggtttggctgcgcgggggccgcgcgtcggagtttccccctttctagggtgaggatggttctacacagccacccggagttccttagttgaaaggtgcgccctgctgtgacagcatggacaccacgttgctgaaaacatgctttgggactgccactgaatttatcttttgcggttttatgacaaagttattagtagtttcccttttttgaattagtattttgaagttaatatcacaatgagttcaggcttatggagccaagaaaaagtcacttcaccctactgggaagagcggattttttacttgcttcttcaagaatgcagcgttacagacaaacaaacacaaaagctccttaaagtaccgaagggaagtataggacagtatattcaagatcgttctgtggggcattcaaggattccttctgcaaaaggcaagaaaaatcagattggattaaaaattctagagcaacctcatgcagttctctttgttgatgaaaaggatgttgtagagataaatgaaaagttcacagagttacttttggcaattaccaattgtgaggagaggttcagcctgtttaaaaacagaaacagactaagtaaaggcctccaaatagacgtgggctgtcctgtgaaagtacagctgagatctggggaagaaaaatttcctggagttgtacgcttcagaggacccctgttagcagagaggacagtctccggaatattctttggagttgaattgctggaagaaggtcgtggtcaaggtttcactgacggggtgtaccaagggaaacagctttttcagtgtgatgaagattgtggcgtgtttgttgcattggacaagctagaactcatagaagatgatgacactgcattggaaagtgattacgcaggtcctggggacacaatgcaggtcgaacttcctcctttggaaataaactccagagtttctttgaaggttggagaaacaatagaatctggaacagttatattctgtgatgttttgccaggaaaagaaagcttaggatattttgttggtgtggacatggataaccctattggcaactgggatggaagatttgatggagtgcagctttgtagttttgcgtgtgttgaaagtacaattctattgcacatcaatgatatcatcccagagagtgtgacgcaggaaaggaggcctcccaaacttgcctttatgtcaagaggtgttggggacaaaggttcatccagtcataataaaccaaaggctacaggatctacctcagaccctggaaatagaaacagatctgaattattttataccttaaatgggtcttctgttgactcacaaccacaatccaaatcaaaaaatacatggtacattgatgaagttgcagaagaccctgcaaaatctcttacagagatatctacagactttgaccgttcttcaccaccactccagcctcctcctgtgaactcactgaccaccgagaacagattccactctttaccattcagtctcaccaagatgcccaataccaatggaagtattggccacagtccactttctctgtcagcccagtctgtaatggaagagctaaacactgcacccgtccaagagagtccacccttggccatgcctcctgggaactcacatggtctagaagtgggctcattggctgaagttaaggagaaccctcctttctatggggtaatccgttggatcggtcagccaccaggactgaatgaagtgctcgctggactggaactggaagatgagtgtgcaggctgtacggatggaaccttcagaggcactcggtatttcacctgtgccctgaagaaggcgctgtttgtgaaactgaagagctgcaggcctgactctaggtttgcatcattgcagccggtttccaatcagattgagcgctgtaactctttagcatttggaggctacttaagtgaagtagtagaagaaaatactccaccaaaaatggaaaaagaaggcttggagataatgattgggaagaagaaaggcatccagggtcattacaattcttgttacttagactcaaccttattctgcttatttgcttttagttctgttctggacactgtgttacttagacccaaagaaaagaacgatgtagaatattatagtgaaacccaagagctactgaggacagaaattgttaatcctctgagaatatatggatatgtgtgtgccacaaaaattatgaaactgaggaaaatacttgaaaaggtggaggctgcatcaggatttacctctgaagaaaaagatcctgaggaattcttgaatattctgtttcatcatattttaagggtagaacctttgctaaaaataagatcagcaggtcaaaaggtacaagattgttacttctatcaaatttttatggaaaaaaatgagaaagttggcgttcccacaattcagcagttgttagaatggtcttttatcaacagtaacctgaaatttgcagaggcaccatcatgtctgattattcagatgcctcgatttggaaaagactttaaactatttaaaaaaatttttccttctctggaattaaatataacagatttacttgaagacactcccagacagtgccggatatgtggagggcttgcaatgtatgagtgtagagaatgctacgacgatccggacatctcagctggaaaaatcaagcagttttgtaaaacctgcaacactcaagtccaccttcatccgaagaggctgaatcataaatataacccagtgtcacttcccaaagacttacccgactgggactggagacacggctgcatcccttgccagaatatggagttatttgctgttctctgcatagaaacaagccactatgttgcttttgtgaagtatgggaaggacgattctgcctggctcttctttgacagcatggccgatcgggatggtggtcagaatggcttcaacattcctcaagtcaccccatgcccagaagtaggagagtacttgaagatgtctctggaagacctgcattccttggactccaggagaatccaaggctgtgcacgaagactgctttgtgatgcatatatgtgcatgtaccagagtccaacaatgagtttgtacaaataactggggtcatcgggaaaggcaaagaaactgaaggcagagtcctaacgttgcatcttattcgagctggcagttctgttcacgtccattgccggcaatggatgtctttgtggtgatgatccttcagaaaaggatgcctctgtttaaaaacaaattgcttttgtgtccctgaagtatttaataagaagcattttgcactctagaaagtatgtttgtgttggttttttaagaagtctaaatgaagttattaatacctgaagctttaagttaagtgcattgatcatatgatatttttggaagcatacaattttaattgtggaagtttaaagcctcttttagtccattgagaatgtaaataaatgtgtcttctttatggaccaaggatatgaaatcatttttcttttgtagctaacggttgccttgaggaagaaataatttggttttattaagagtctactctcaatccagttattagagatgtactgagtttgatttgttaatcctttctatatactgctgatcttgcatgtctacaatctgctcagtttttctgtgtttctgcaatagtggtcagaaaaatacttaaattcccttaatggtgttgttttctatttgttctggttttgagataaatgagtgattctgtccccaaatgtccatttttgaagtgattttcctggaggattagggtatttagcagttgaagctcttcattcatagtagttactgtcagctaacaggttttttaaggcttttaactattaatattttatggaatggggcaaagtaaattgatgaaagaattggagtgataatagtcctttacaaacatacagtccataagaaaatgaatttggcatatagaattattacaatttcctgggagagatggatatttaaacctctattattttagacaagactgtctagaacttaagtttgatctgtcagccagtactcccattaaattcagtgtagtttcacttgatagaatcagatatgttatcgaaatgttagcagcagcttcatcctccttctgattaaagtaagtagaaatgggatgttttgtttaataacagccatagtgtgtgtttagaccacagcggatgttgtagaccaggaccatagatgatacatgtcagtgctgtggaatgtgcattctctgagtgttgttttgtggtatcattgtctttcctgaatgactttctaactgtgcagaaaggcagaaaagtcatcatatgtatatgtcatatgactttataaaatatttaatgtgacaaaaagtggaaagaatctttacaaaccctgcaattacttttttaaaggcacttttactctttggttttatcattccattttgctaatatttactagctttataaattacagtaaggtacaaaaactcatcttgtaatattttcatttttgaagtgaaaaagtacatatattttgcacaaggttttatactgctaagtgcttggttggggtggtgagatgatgattagatcaggggtgaggctgagagactctgggtttagggctagccctgcctccatctcccttgggtaaaatgaagggtgtggggtaaaagatgcataaggccttttctagctctgacagcctagaagtccaatcaccctgtaataaatatgtgttgaatgaagaaatgggtgaatgagcttgtcaatgtgattttaaaaaattgactacctggaggaatgattaggaatctaaatgaagccagccctcggtatctgcaggtttctcatccatggattcaaccaactgcaaatggaaaatacgattttttttaaaaaaaggatggttacatccgtattgaacatgtacagacttttttcttgtcattattctctgaacaatacaagaactctttatgtagcatttacatttattaggtattataagtaatctagagattatttaattaaaatatacaggaggatgtgtgtcagttatatgcaaattctgtaccattttgtatcagggaattgagcatcttcagatgttggtatctgcagggatcctggaaccaaacccctgcagatactaagggctgacgatctaggtaagactggatttaacagttggaaaaaaaaaaaaaaaaggagagagaagacagttcctttcctgtagaaattaaaacaaaatacaaattgaggaagctctgctacccaggctgtcatggtagagaacttgaagaagacctgtttggatggacacctggtttcaaaagtcaggtgtggagactgttaaatgggagggcctcatccataaatgatttctggcaacgtcttcttcaggtggagcttgacgtctttttaatgttacttggggagggagtgctcattaagggatgccagggccagctctggtggttcctggggaggctgcgtccttccctgcttctgcatgtcatgaggcagcaggaaggtttcccctgcacctgtctgtcctggctccctctgggtagccccctactgttctgtgcttcagcacagcctggtttgtcaagaggcacatagttggggctgggctgcatggcacaggggcttatgtgcctgctggttatttaattttcagccttaagttttctttaatattttcctgttggctatttaaaggttttggttatcttttattccttatctacaatcaagatgacaatgtaattgaattatcttatttataacacggttcgtgattcatgattcatgattacaagtagaaaatatgtcatgttcctcacctccaaataaatatgtgtgtgtctgtgtgtgtgtatatatgtatgtggcggagagggagagagtggggaaggagagcagtgttatcatacatagagaggctaaatgtgtcccatccctcactgtcagctttataaaggagtttgactccatccacagaagaatgttttataagactaggaaaacacgttgaaaactaggataaacagcaacaaaaatcaactaaatatgttgttactgttgctaaggattttctccttagaataatttaggatttttaaaaatttctgtttgccaaatgctgtagataaatggccagattcttcctatccctaggattcctttattattttttttcacagattttgagaacaagggggagagatagtatggaagattaagattccattaatcttatagaactgtgttgtcacccaaattcctgcttgtttgaacatggcatcttcatagattcaggattcactaccctctatagctggatcttgaaaattatctggccagataattttgcatctgcttggatgattgtagactgagatgtgagtggaggataaagtattagacttttgctgagtaactgccaaccaagaagtatttatcggacacttactaggtgcctaggattgtatcagagggaatatgaaatgtgtccctgccctacctagttttaacgacagaatatctattaaaggctacttagctgaagggtaagggtgacaggtctaggggaagctttgggaggtggtgtgctgtgacagaaaaagtggcagagtagggacgagagacctgcattctagccctgtttctgtcacttgctctgtacacttagacaacagcttgacctcttgagctttagtttcctcctctgcataatgagagggttagactactgaattgtatgggaaaaaaatacaaattcctgggtcctaggccatgcctgctgaatccgactgttcaggaagaggcctaggaaatctgtgagggaatccccaggggaatctcgtgaccagccaggtgtgaaatctgctaactggaagatctcaaagcttccttcactttttgtgattttgtggtcatgtaacgttactgtattattctacgtaaatgtgggtacttggatgtttatcatactgtttctctgtgtttacatactaatttgtgtaagaaatgcattttagtctgtgtacctcaacctgctgtttgtttcctagaggtgttagtagtctttaaatacaagtaagacttaagaggatatttgatgttatttacctggatattttcttccccttttatttatttagaggaaattgagattctaggagccaaaaaaatgaaaacaaaattctaaggcaaagttaaagaaaaaaattacattatttcttaccatttgctactttataatgaaaatttaaaaattatatgggaagatttttctctgggataacaaatccttgtcataaagtaagaggtctttttaaagtaggtaggctataaggcctgtaatttaaaataatactcctttctctagggtttggtgcaattctccattaatgaagataacatttgaattccccaaagcaggtgaggagtcggggaggagaaagcgatgttaaaatgaaaactcactgcaaaagaggaggcagaggaagaaggaatgtaaaccccttaaagcagatgtgtgtggggccttatgaagaccaggattctgcgggtgtcaggggattgcccctcttgacagagactagggttttagactgaggcttcctgcagggtgttcgcattgcccttctccgttccccttcagacctttctggggagaagaggtgggaggaggggagaaagactgttcatcttattctgaatcctggagcagctgaaggttttctcttgagtcaggatgcagtggtaatgcattaaccagcaagtgtggccaaggataatgaaaaagtgggaaaggaaggtcctcctcctccctgattgtagcatccagcagtctctgtagccaggttactcaagaaccacatttgatttcctggccctttgccttggcagtgatggcatttttatttcactgtgttttaaagtcttcatttatttttataacatgggttagggagaagggccacaaatggagggattgtcctttcaagcaccacagcttcagataaaattagtactttcaaatattgtccactttaacttaaaaaattctagagggattatattggagactcaactgcccttggttttagtttataaaatggcctagtactgtggaattttaattttagaaagtcttagcatcagatcataaacattcattaaaagaactcacatcccatctgaaacttcccaggggagttgggattcttagtagattggtagaaaggggctcattttctactgcatttcccatttttggtatcttgttcagcatgttttatttttatttcttgtctgcagaacatcctatatttatgagaacattctttaagaagaccaccacatagaataccccttcctatcagctcgctctgatttagccttaattttgttaaattttttagagatgaatgaagtgctgctgtggaaagaaatgtacatatactatttctgtatcattaaaattacatttttatggttc

SEQ ID NO: 40 - DNA, 8623 bp, Homo sapiens
aggtagcaggtttggctgcgcgggggccgcgcgtcggaggtaaatactagggcggtgggtgtggggagccggggccggcccgggacgcgggctggggagccggggcgaggggcgacgccccgccgcccgagtttccccctttctagggtgaggatggttctacacagccacccggagttccttagttgaaaggtgcgccctgctgtgacagcatggacaccacgttgctgaaaacatgctttgggactgccactgaatttatcttttgcggttttatgacaaagttattagtagtttcccttttttgaattagtattttgaagttaatatcacaatgagttcaggcttatggagccaagaaaaagtcacttcaccctactgggaagagcggattttttacttgcttcttcaagaatgcagcgttacagacaaacaaacacaaaagctccttaaagtaccgaagggaagtataggacagtatattcaagatcgttctgtggggcattcaaggattccttctgcaaaaggcaagaaaaatcagattggattaaaaattctagagcaacctcatgcagttctctttgttgatgaaaaggatgttgtagagataaatgaaaagttcacagagttacttttggcaattaccaattgtgaggagaggttcagcctgtttaaaaacagaaacagactaagtaaaggcctccaaatagacgtgggctgtcctgtgaaagtacagctgagatctggggaagaaaaatttcctggagttgtacgcttcagaggacccctgttagcagagaggacagtctccggaatattctttggagttgaattgctggaagaaggtcgtggtcaaggtttcactgacggggtgtaccaagggaaacagctttttcagtgtgatgaagattgtggcgtgtttgttgcattggacaagctagaactcatagaagatgatgacactgcattggaaagtgattacgcaggtcctggggacacaatgcaggtcgaacttcctcctttggaaataaactccagagtttctttgaaggttggagaaacaatagaatctggaacagttatattctgtgatgttttgccaggaaaagaaagcttaggatattttgttggtgtggacatggataaccctattggcaactgggatggaagatttgatggagtgcagctttgtagttttgcgtgtgttgaaagtacaattctattgcacatcaatgatatcatcccagagagtgtgacgcaggaaaggaggcctcccaaacttgcctttatgtcaagaggtgttggggacaaaggttcatccagtcataataaaccaaaggctacaggatctacctcagaccctggaaatagaaacagatctgaattattttataccttaaatgggtcttctgttgactcacaaccacaatccaaatcaaaaaatacatggtacattgatgaagttgcagaagaccctgcaaaatctcttacagagatatctacagactttgaccgttcttcaccaccactccagcctcctcctgtgaactcactgaccaccgagaacagattccactctttaccattcagtctcaccaagatgcccaataccaatggaagtattggccacagtccactttctctgtcagcccagtctgtaatggaagagctaaacactgcacccgtccaagagagtccacccttggccatgcctcctgggaactcacatggtctagaagtgggctcattggctgaagttaaggagaaccctcctttctatggggtaatccgttggatcggtcagccaccaggactgaatgaagtgctcgctggactggaactggaagatgagtgtgcaggctgtacggatggaaccttcagaggcactcggtatttcacctgtgccctgaagaaggcgctgtttgtgaaactgaagagctgcaggcctgactctaggtttgcatcattgcagccggtttccaatcagattgagcgctgtaactctttagcatttggaggctacttaagtgaagtagtagaagaaaatactccaccaaaaatggaaaaagaaggcttggagataatgattgggaagaagaaaggcatccagggtcattacaattcttgttacttagactcaaccttattctgcttatttgcttttagttctgttctggacactgtgttacttagacccaaagaaaagaacgatgtagaatattatagtgaaacccaagagctactgaggacagaaattgttaatcctctgagaatatatggatatgtgtgtgccacaaaaattatgaaactgaggaaaatacttgaaaaggtggaggctgcatcaggatttacctctgaagaaaaagatcctgaggaattcttgaatattctgtttcatcatattttaagggtagaacctttgctaaaaataagatcagcaggtcaaaaggtacaagattgttacttctatcaaatttttatggaaaaaaatgagaaagttggcgttcccacaattcagcagttgttagaatggtcttttatcaacagtaacctgaaatttgcagaggcaccatcatgtctgattattcagatgcctcgatttggaaaagactttaaactatttaaaaaaatttttccttctctggaattaaatataacagatttacttgaagacactcccagacagtgccggatatgtggagggcttgcaatgtatgagtgtagagaatgctacgacgatccggacatctcagctggaaaaatcaagcagttttgtaaaacctgcaacactcaagtccaccttcatccgaagaggctgaatcataaatataacccagtgtcacttcccaaagacttacccgactgggactggagacacggctgcatcccttgccagaatatggagttatttgctgttctctgcatagaaacaagccactatgttgcttttgtgaagtatgggaaggacgattctgcctggctcttctttgacagcatggccgatcgggatggtggtcagaatggcttcaacattcctcaagtcaccccatgcccagaagtaggagagtacttgaagatgtctctggaagacctgcattccttggactccaggagaatccaaggctgtgcacgaagactgctttgtgatgcatatatgtgcatgtaccagagtccaacaatgagtttgtacaaataactggggtcatcgggaaaggcaaagaaactgaaggcagagtcctaacgttgcatcttattcgagctggcagttctgttcacgtccattgccggcaatggatgtctttgtggtgatgatccttcagaaaaggatgcctctgtttaaaaacaaattgcttttgtgtccctgaagtatttaataagaagcattttgcactctagaaagtatgtttgtgttggttttttaagaagtctaaatgaagttattaatacctgaagctttaagttaagtgcattgatcatatgatatttttggaagcatacaattttaattgtggaagtttaaagcctcttttagtccattgagaatgtaaataaatgtgtcttctttatggaccaaggatatgaaatcatttttcttttgtagctaacggttgccttgaggaagaaataatttggttttattaagagtctactctcaatccagttattagagatgtactgagtttgatttgttaatcctttctatatactgctgatcttgcatgtctacaatctgctcagtttttctgtgtttctgcaatagtggtcagaaaaatacttaaattcccttaatggtgttgttttctatttgttctggttttgagataaatgagtgattctgtccccaaatgtccatttttgaagtgattttcctggaggattagggtatttagcagttgaagctcttcattcatagtagttactgtcagctaacaggttttttaaggcttttaactattaatattttatggaatggggcaaagtaaattgatgaaagaattggagtgataatagtcctttacaaacatacagtccataagaaaatgaatttggcatatagaattattacaatttcctgggagagatggatatttaaacctctattattttagacaagactgtctagaacttaagtttgatctgtcagccagtactcccattaaattcagtgtagtttcacttgatagaatcagatatgttatcgaaatgttagcagcagcttcatcctccttctgattaaagtaagtagaaatgggatgttttgtttaataacagccatagtgtgtgtttagaccacagcggatgttgtagaccaggaccatagatgatacatgtcagtgctgtggaatgtgcattctctgagtgttgttttgtggtatcattgtctttcctgaatgactttctaactgtgcagaaaggcagaaaagtcatcatatgtatatgtcatatgactttataaaatatttaatgtgacaaaaagtggaaagaatctttacaaaccctgcaattacttttttaaaggcacttttactctttggttttatcattccattttgctaatatttactagctttataaattacagtaaggtacaaaaactcatcttgtaatattttcatttttgaagtgaaaaagtacatatattttgcacaaggttttatactgctaagtgcttggttggggtggtgagatgatgattagatcaggggtgaggctgagagactctgggtttagggctagccctgcctccatctcccttgggtaaaatgaagggtgtggggtaaaagatgcataaggccttttctagctctgacagcctagaagtccaatcaccctgtaataaatatgtgttgaatgaagaaatgggtgaatgagcttgtcaatgtgattttaaaaaattgactacctggaggaatgattaggaatctaaatgaagccagccctcggtatctgcaggtttctcatccatggattcaaccaactgcaaatggaaaatacgattttttttaaaaaaaggatggttacatccgtattgaacatgtacagacttttttcttgtcattattctctgaacaatacaagaactctttatgtagcatttacatttattaggtattataagtaatctagagattatttaattaaaatatacaggaggatgtgtgtcagttatatgcaaattctgtaccattttgtatcagggaattgagcatcttcagatgttggtatctgcagggatcctggaaccaaacccctgcagatactaagggctgacgatctaggtaagactggatttaacagttggaaaaaaaaaaaaaaaaggagagagaagacagttcctttcctgtagaaattaaaacaaaatacaaattgaggaagctctgctacccaggctgtcatggtagagaacttgaagaagacctgtttggatggacacctggtttcaaaagtcaggtgtggagactgttaaatgggagggcctcatccataaatgatttctggcaacgtcttcttcaggtggagcttgacgtctttttaatgttacttggggagggagtgctcattaagggatgccagggccagctctggtggttcctggggaggctgcgtccttccctgcttctgcatgtcatgaggcagcaggaaggtttcccctgcacctgtctgtcctggctccctctgggtagccccctactgttctgtgcttcagcacagcctggtttgtcaagaggcacatagttggggctgggctgcatggcacaggggcttatgtgcctgctggttatttaattttcagccttaagttttctttaatattttcctgttggctatttaaaggttttggttatcttttattccttatctacaatcaagatgacaatgtaattgaattatcttatttataacacggttcgtgattcatgattcatgattacaagtagaaaatatgtcatgttcctcacctccaaataaatatgtgtgtgtctgtgtgtgtgtatatatgtatgtggcggagagggagagagtggggaaggagagcagtgttatcatacatagagaggctaaatgtgtcccatccctcactgtcagctttataaaggagtttgactccatccacagaagaatgttttataagactaggaaaacacgttgaaaactaggataaacagcaacaaaaatcaactaaatatgttgttactgttgctaaggattttctccttagaataatttaggatttttaaaaatttctgtttgccaaatgctgtagataaatggccagattcttcctatccctaggattcctttattattttttttcacagattttgagaacaagggggagagatagtatggaagattaagattccattaatcttatagaactgtgttgtcacccaaattcctgcttgtttgaacatggcatcttcatagattcaggattcactaccctctatagctggatcttgaaaattatctggccagataattttgcatctgcttggatgattgtagactgagatgtgagtggaggataaagtattagacttttgctgagtaactgccaaccaagaagtatttatcggacacttactaggtgcctaggattgtatcagagggaatatgaaatgtgtccctgccctacctagttttaacgacagaatatctattaaaggctacttagctgaagggtaagggtgacaggtctaggggaagctttgggaggtggtgtgctgtgacagaaaaagtggcagagtagggacgagagacctgcattctagccctgtttctgtcacttgctctgtacacttagacaacagcttgacctcttgagctttagtttcctcctctgcataatgagagggttagactactgaattgtatgggaaaaaaatacaaattcctgggtcctaggccatgcctgctgaatccgactgttcaggaagaggcctaggaaatctgtgagggaatccccaggggaatctcgtgaccagccaggtgtgaaatctgctaactggaagatctcaaagcttccttcactttttgtgattttgtggtcatgtaacgttactgtattattctacgtaaatgtgggtacttggatgtttatcatactgtttctctgtgtttacatactaatttgtgtaagaaatgcattttagtctgtgtacctcaacctgctgtttgtttcctagaggtgttagtagtctttaaatacaagtaagacttaagaggatatttgatgttatttacctggatattttcttccccttttatttatttagaggaaattgagattctaggagccaaaaaaatgaaaacaaaattctaaggcaaagttaaagaaaaaaattacattatttcttaccatttgctactttataatgaaaatttaaaaattatatgggaagatttttctctgggataacaaatccttgtcataaagtaagaggtctttttaaagtaggtaggctataaggcctgtaatttaaaataatactcctttctctagggtttggtgcaattctccattaatgaagataacatttgaattccccaaagcaggtgaggagtcggggaggagaaagcgatgttaaaatgaaaactcactgcaaaagaggaggcagaggaagaaggaatgtaaaccccttaaagcagatgtgtgtggggccttatgaagaccaggattctgcgggtgtcaggggattgcccctcttgacagagactagggttttagactgaggcttcctgcagggtgttcgcattgcccttctccgttccccttcagacctttctggggagaagaggtgggaggaggggagaaagactgttcatcttattctgaatcctggagcagctgaaggttttctcttgagtcaggatgcagtggtaatgcattaaccagcaagtgtggccaaggataatgaaaaagtgggaaaggaaggtcctcctcctccctgattgtagcatccagcagtctctgtagccaggttactcaagaaccacatttgatttcctggccctttgccttggcagtgatggcatttttatttcactgtgttttaaagtcttcatttatttttataacatgggttagggagaagggccacaaatggagggattgtcctttcaagcaccacagcttcagataaaattagtactttcaaatattgtccactttaacttaaaaaattctagagggattatattggagactcaactgcccttggttttagtttataaaatggcctagtactgtggaattttaattttagaaagtcttagcatcagatcataaacattcattaaaagaactcacatcccatctgaaacttcccaggggagttgggattcttagtagattggtagaaaggggctcattttctactgcatttcccatttttggtatcttgttcagcatgttttatttttatttcttgtctgcagaacatcctatatttatgagaacattctttaagaagaccaccacatagaataccccttcctatcagctcgctctgatttagccttaattttgttaaattttttagagatgaatgaagtgctgctgtggaaagaaatgtacatatactatttctgtatcattaaaattacatttttatggttc

SEQ ID NO: 41 - protein, 955 aa, Mus musculus
MSSGLWSQEKVTSPYWEERIFYLLLQECSVTDKQTQKLLKVPKGSIGQYIQDRSVGHSRVPSTKGKKNQIGLKILEQPHAVLFVDEKDVVEINEKFTELLLAITNCEERLSLFRNRLRLSKGLQVDVGSPVKVQLRSGEEKFPGVVRFRGPLLAERTVSGIFFGVELLEEGRGQGFTDGVYQGKQLFQCDEDCGVFVALDKLELIEDDDNGLESDFAGPGDTMQVEPPPLEINSRVSLKVGESTESGTVIFCDVLPGKESLGYFVGVDMDNPIGNWDGRFDGVQLCSFASVESTILLHINDIIPALSDSVTQERRPPKLAFMSRGVGDKGSSSHNKPKVTGSTSDPGSRNRSELFYTLNGSSVDSQQSKSKNPWYIDEVAEDPAKSLTEMSSDFGHSSPPPQPPSMNSLSSENRFHSLPFSLTKMPNTNGSMAHSPLSLSVQSVMGELNSTPVQESPPLPISSGNAHGLEVGSLAEVKENPPFYGVIRWIGQPPGLSDVLAGLELEDECAGCTDGTFRGTRYFTCALKKALFVKLKSCRPDSRFASLQPVSNQIERCNSLAFGGYLSEVVEENTPPKMEKEGLEIMIGKKKGIQGHYNSCYLDSTLFCLFAFSSALDTVLLRPKEKNDIEYYSETQELLRTEIVNPLRIYGYVCATKIMKLRKILEKVEAASGFTSEEKDPEEFLNILFHDILRVEPLLKIRSAGQKVQDCNFYQIFMEKNEKVGVPTIQQLLEWSFINSNLKFAEAPSCLIIQMPRFGKDFKLFKKIFPSLELNITDLLEDTPRQCRICGGLAMYECRECYDDPDISAGKIKQFCKTCSTQVHLHPRRLNHSYHPVSLPKDLPDWDWRHGCIPCQKMELFAVLCIETSHYVAFVKYGKDDSAWLFFDSMADRDGGQNGFNIPQVTPCPEVGEYLKMSLEDLHSLDSRRIQGCARRLLCDAYMCMYQSPTMSLYK

SEQ ID NO: 42 - DNA, 8023 bp, Mus musculus
agttcggaggcggggcaggtgggggcgggcccaggtagcaggttcggctgcgcgggggcccgcgcgcctgaggcactttgaattgctgtctttttacaacatggatgccaggttgctaaaatcttgctttgggacttacagcgagttcatttttttggattttatgacagatttactagtggctcctttttgccagtcaatgttctgaaagttactgtcacaatgagttcaggcctgtggagccaagagaaagttacttcaccctactgggaagaacggattttttatctgcttcttcaagaatgcagtgtaacagacaaacaaactcagaagctgctgaaagtacccaaagggagtataggacagtacatccaagaccgttctgtggggcattcaagagttccttccacaaaaggcaagaaaaatcagattggattaaaaatcttggagcaaccgcatgcagttctgtttgttgatgaaaaggatgttgtagaaataaatgaaaaattcacagagttactgttggcaattaccaactgtgaggagaggctcagcctatttagaaacagactccgactaagtaaaggcctccaggtagacgtgggcagtcctgtgaaagtacagctgcgatctggggaagagaaatttccaggagttgtacgcttcagaggacctttattagcggagaggacggtgtcggggattttctttggagtagaattattggaagaaggtcgtggtcaaggtttcacggatggggtatatcaagggaagcagcttttccagtgtgatgaggactgtggcgtgtttgttgcattggacaaactagaacttatagaagatgatgacaatggattggaaagtgattttgcaggcccaggagatacaatgcaggttgaacctccccctttggaaataaactccagagtttctttgaaggttggagaaagtacagaatctggaacagtaatattctgtgatgttttaccaggaaaagagagtctaggatattttgttggtgtggacatggataaccctattggcaactgggatggaaggtttgatggagtacagctctgtagttttgcaagtgttgaaagtacaattctcctgcacatcaatgacatcatcccagctttatcagatagcgtgacacaggaaaggaggcctcccaaacttgcctttatgtcaagaggtgtaggtgacaaaggttcatctagtcataataaaccaaaggttacaggatctacctcagaccctggaagtagaaacagatctgaattattttataccttaaatgggtcatctgttgactcacaacaatccaagtccaaaaatccatggtacattgatgaagttgcagaagaccctgcaaagtcacttacagagatgtcttcggacttcggacattcatctcctccaccgcagcctccttccatgaactccttgtctagcgagaacagattccactccttacccttcagcctgacaaagatgcccaatactaatggcagcatggctcatagtccactctctctgtcagtgcagtctgtgatgggggagctgaacagcacacctgtccaggagagtccacccttgcccatctcttctgggaatgcacacgggctagaggtgggctcactggctgaagtaaaagagaaccccccgttctatggggttatccgttggattggccagccaccagggctcagtgacgtgctagctggactggaactggaagatgaatgcgcaggctgtacagatggaactttcaggggcacgcggtatttcacgtgtgccctgaagaaggcactgtttgtgaaactgaagagctgcagaccggactctaggtttgcatccttgcagcctgtttccaatcaaattgaaaggtgtaactctttagcatttgggggctatttaagtgaagtagtagaagaaaatactccacctaaaatggaaaaggaaggtttagagataatgattggaaagaagaaaggcatccagggccattacaattcttgttacttagactcaactttattctgcttatttgcttttagttctgccctggacactgtgttacttagacccaaagagaagaatgatatagagtattacagtgagactcaggagctactgaggacagagatagtcaatcctctgagaatatatggatatgtgtgtgccacaaagattatgaaactgaggaaaatacttgaaaaagttgaggctgcatcaggttttacctctgaagaaaaagatcctgaagaattcttaaatatcctgtttcatgatattttaagggttgaaccattgttaaaaataagatcagcaggtcaaaaagttcaagactgtaacttctatcaaatttttatggaaaaaaatgagaaagttggagtacccacaattcagcagttattagaatggtcttttatcaacagcaacctgaaatttgcagaggcaccatcatgcttgattatccagatgcctcggtttggaaaagactttaaactatttaaaaaaatttttccttccctggaattaaatataacagatttacttgaagacactcccaggcagtgccgcatctgtggaggacttgcgatgtacgagtgcagggagtgctatgacgatccggacatctcagctgggaagatcaagcagttctgtaagacctgcagcactcaggttcaccttcatcccaggaggttgaatcattcttatcatccagtatcacttcccaaagacttgcctgactgggactggagacacggctgcatcccctgtcagaagatggagttatttgctgttctctgcatagaaacgagccactatgttgcttttgtgaagtatgggaaggatgactctgcctggcttttctttgacagcatggcggatcgagatggtggtcagaatggcttcaacattccacaagtgacgccctgcccagaagtgggagagtacttgaagatgtctctggaggacctgcactctttggattccagaaggattcaaggctgtgcgcgcagacttctttgcgatgcatacatgtgcatgtaccagagtccaaccatgagcctgtacaaataaccaggttatccgcagtgggcagagacagcacaaaccttagtgctgtgtcttcctccagcttgtggttctgtccggcacccattgccggcaatgggtgtctcttttgatgatggcccttcagcaaaggattcctgtgtttccaaacaaattgcttttgtgttcctgaagtatttaataagaagcattttgcactctaggaagtatgtttgtgttgattttttaagaagtctaaatggatttattaatatctgacactttaagttaactgcattgatgatacgatatttttggaagcatacaattttaattgtgacgtctaaaacctcttttagtccattgaggatgtaaataaatatttcttcttatggaccagggatatgaaatcattttccttttgtagctaatgattgccttgagaaagacataatatgattttattaagagtctgcctttactccaattgttgaaggtgtgctgagtttaattgttaattttcctatatagtgctgatttcctgcatgtctgtactttgctgagtttctgtgttcctgcagcagtggtcagaacatgctgaaattcctttcatggtgttattttttatttgttctagttttgagataaatgagtgactctgtcataaaatgtccatttttgatttgttcttgaagaattagggtacaaaacaagtatcgtagtcatagtaacaactgttactgaacaggtcttctgtaggggctcctaagtatgagacaacatgcagtggtttctgaaatgactgagcgataagtaaattatatattctttttttaaaagatttattttatttatatgagtacactgtagctgtcttcagacacaccagaagagggcatcagatcttattacagatggttgtgagccaccatgtggttgctgggaattgaactcaggacctttggaagggcagtcagtgctcttaactgctgagctatctctctagccccaaattatatattcttatacagacccacagtccagagaaagtgaagctggctcagagttagcaccaacatttccctgaaaaaatgtgttaggtgtttaaaaatctcatcgtaaataaattattttatttttagtttaagtgaatttctcttaatttaaataagactaatatttttgtttgactgaagacccttgtactctctggtgtcagtttggtgtcacttgaaacaatgacaaccacgtcagcatgttactgcaatgtcagtgatggtttcaaaggagaaaatggatgtcatcttaggactgcttgtgtgtgtatagaccgtggtggatgctgtagagtgcttgttgtctgagtgttgttttgtggtatggctgtctctctgaataatgtcaaccttacagaaagagaaggtgttatatgtgtaacatatggctttataaaagatttaatgtcaaaagagtctataaatattgctgttatttttaaaggcactttactccttggttttattattccattttgctggcgtgtattagctttatataaatcacaacacacaaaactcgtccatcttgtaatctttgtacttttgcaatggaaaagtgtgtatactgtgcacagacttttatatagctgagtgcctagactgggtggagaggccatgagtagatagcagtaaagtcctccagctataggctagccttgtcttcatcatcctgggacaggtgagaacggtgagcacaggtgagtctgagcttgcacactgaccagaattcagtcaccctgtaataagtatgtatttaatgagtctgttagtgtggtagtgtgattttgaaaggtttaactaaggaatggtgaggtctttgtttataggcaagcctggcttaacagtgaaggggagggggctgagggaaagctttttttttttttaatttcagaaatggaagcaaaatccatgtgcaaagagactagctacttggctttcagagcagaggactcaaagcttgtttgcagtggttctgaagttggggtggcactttgtgagtgaggaggaccatgtccacaggcaatccctaagcaagatcgtctcgaaattgcactgtgttctttagcagtagtggctgggtaggggcacacagcggaacacccacttgcagtggctggggaggcctgagaagcacaggccagcgggagctctctcccatgccttcttgtccctctggacagcccctcctcattccttgtcccagcagagcctggtttgcccctggggcaatagggttggactgtgtggtgcagatagccctcgtgcctgtggattactttaattttgtaatcttaggttttctgtacggttttcccattgccttttaaaatattttgagtgtccttctccttatcaagatggtagtgtagtaggattgttttactcacaacatggtttaggagccatagctacaaataggaaatataagaattctatttctctactgtgaggcaaatctgtatatatgtatggagagtcaggaggatgaacaagggagaggaaaggggcccaggcacatttttaatccaaaagctacaaagtgtcgagctcctcagggccagcttcacagagagtccagctcagttcacaaagtattttctaagttggggaaatgcattgagaaacagagcgagatatctgtgtagtgcagctgtgggtggttgctatgcattttatcctcagagtagcctggggtttttattttatgtttgccaagtgcttaggtaaatggccgggctcctcttacctctgacttctggagcgtgtccattctctggttgactgaatgtggcattttcataaatctaggagcagcagccctcccaactgcaatttagtaagcaggtagataagcttgcatctgtttgcatcttcctgcactgagttgggagtgcaaaaccccgtacttggcctggtgagtaactgccaacaagtatttatgggtcatttgctaggtggctagcatacggcacggggctaatgagacctggggcctgctctcatatagcttactgctcagtttctataccagagcatccgttggtgcttcttagcctaggcatgggtgacaggcatgtctggggaccactttgggagacaacctagtgtaaccttattgcaaaggactaagagacaaaaatcctgggttctttctccatcacttgctctgtagctgaatgaacaccacctcgatttcttgagtttcagtgtcttgttctgtctgatgaaagggttggatatgtagttggataagcggtacaaattgttagggttccaggccatgcctgatacatctgagtttttgctggaggtaggaaatttcaactcagaagctcatccacagaaaacagtgaccagcagcaggtatggagccataaggctggaaggcttcaaagaccccttcttctgttactgttatccacggcagttattctgtgtaggtgtgaaggagtggctactgactctgtgtggatcagcgtttacatatgacctgatgggagtgcatctggtatgtgttcctcccccctgtattgctggagagattggcaatcgtaggtatacacggaagtgtcgggatgttcagtggcgcgcacttggatgtttcttttcactttatttctatagaaacaggttccaggagccagagaagtaggggaaaaaatccctgagctcatctctctctactacttaataatgcaaacatttaaattcttgggaagattttcccgccccactccctacccccagtcacaggtcagaataggatttccttcttagagaaggtaggtcatggggcctgcgattaaaaaaacaaaaacataaaacaaacaaacaaacaaggtggctttccttggtctttgtgattccagctcttcaccagtgaaggaattctttagttccatagtgcagcctgggtgggtggagaagaggctgttatagtgggagccagtgagcaaagagggttggaggaggcagaggacagggctcagggaaggcaagcctgtggctctgggcattggccctcttgtcaaaggtgtcagagcatggcttcctgtgaggggctttcccttgctcctgtgtgagaatctgtgcgttgtcttactggatgggggacctggatggagatgttaggggagatagcttagtcttgttctaaactctagacttgctaaaaagggttctcttgagtccaaatgcagtgccagagggtgagaagcagaattggccagtagtgaacagtggtattgtgggtgataagccctttctggtggccttaaatagcagtgctagtccttgagtgccacagggtcccctacctccgtcctggcagtgctgatgggtttgtttcagcatattttggttctcattctcatttagggaaaagggctgttgatgaagggattatcctcccaggcaccaggggcctcagcacagttccctgtgcttttaaacactgttgactttcattagttattctaaagggatttatattggagatacaactccctttggtttcatattatgaaatagctttgtactgtggaatgctaactctggaaagtctccctcataccagaaccaaacattcatttcttttatatatatatatatattatctccctggtactctccagggaggtggttattatcaagctgacccaaaggagtgcagtatgtgttctagttccttttctggtaccttgttcaacattcttttttcctgaagaacatccttttgtttatgaaaacgtgaatagcatcatgtaaaacaatcccttgctatcagaatgctccagtttggccttaattttgtaaaccttggagaggagtggagcgcggctgtgagaagagatgtaccatgtactctttctgtatcattaaagtgcagtttttatgcttct

SEQ ID NO: 43 - protein, 952 aa, Mus musculus
MSSGLWSQEKVTSPYWEERIFYLLLQECSVTDKQTQKLLKVPKGSIGQYIQDRSVGHSRVPSTKGKKNQIGLKILEQPHAVLFVDEKDVVEINEKFTELLLAITNCEERLSLFRNRLRLSKGLQVDVGSPVKVQLRSGEEKFPGVVRFRGPLLAERTVSGIFFGVELLEEGRGQGFTDGVYQGKQLFQCDEDCGVFVALDKLELIEDDDNGLESDFAGPGDTMQVEPPPLEINSRVSLKVGESTESGTVIFCDVLPGKESLGYFVGVDMDNPIGNWDGRFDGVQLCSFASVESTILLHINDIIPDSVTQERRPPKLAFMSRGVGDKGSSSHNKPKVTGSTSDPGSRNRSELFYTLNGSSVDSQQSKSKNPWYIDEVAEDPAKSLTEMSSDFGHSSPPPQPPSMNSLSSENRFHSLPFSLTKMPNTNGSMAHSPLSLSVQSVMGELNSTPVQESPPLPISSGNAHGLEVGSLAEVKENPPFYGVIRWIGQPPGLSDVLAGLELEDECAGCTDGTFRGTRYFTCALKKALFVKLKSCRPDSRFASLQPVSNQIERCNSLAFGGYLSEVVEENTPPKMEKEGLEIMIGKKKGIQGHYNSCYLDSTLFCLFAFSSALDTVLLRPKEKNDIEYYSETQELLRTEIVNPLRIYGYVCATKIMKLRKILEKVEAASGFTSEEKDPEEFLNILFHDILRVEPLLKIRSAGQKVQDCNFYQIFMEKNEKVGVPTIQQLLEWSFINSNLKFAEAPSCLIIQMPRFGKDFKLFKKIFPSLELNITDLLEDTPRQCRICGGLAMYECRECYDDPDISAGKIKQFCKTCSTQVHLHPRRLNHSYHPVSLPKDLPDWDWRHGCIPCQKMELFAVLCIETSHYVAFVKYGKDDSAWLFFDSMADRDGGQNGFNIPQVTPCPEVGEYLKMSLEDLHSLDSRRIQGCARRLLCDAYMCMYQSPTMSLYK

SEQ ID NO: 44 - DNA, 8148 bp, Mus musculus
agttcggaggcggggcaggtgggggcgggcccaggtagcaggttcggctgcgcgggggcccgcgcgcctgagcagtgagctcctatgtcatggattctgaactggcttataatatctggcatggattccttttagcagagcaggcctcaaatccgatagagcatggttgcttgatcccattgcagccctgccactagtggctcctggcactttgaattgctgtctttttacaacatggatgccaggttgctaaaatcttgctttgggacttacagcgagttcatttttttggattttatgacagatttactagtggctcctttttgccagtcaatgttctgaaagttactgtcacaatgagttcaggcctgtggagccaagagaaagttacttcaccctactgggaagaacggattttttatctgcttcttcaagaatgcagtgtaacagacaaacaaactcagaagctgctgaaagtacccaaagggagtataggacagtacatccaagaccgttctgtggggcattcaagagttccttccacaaaaggcaagaaaaatcagattggattaaaaatcttggagcaaccgcatgcagttctgtttgttgatgaaaaggatgttgtagaaataaatgaaaaattcacagagttactgttggcaattaccaactgtgaggagaggctcagcctatttagaaacagactccgactaagtaaaggcctccaggtagacgtgggcagtcctgtgaaagtacagctgcgatctggggaagagaaatttccaggagttgtacgcttcagaggacctttattagcggagaggacggtgtcggggattttctttggagtagaattattggaagaaggtcgtggtcaaggtttcacggatggggtatatcaagggaagcagcttttccagtgtgatgaggactgtggcgtgtttgttgcattggacaaactagaacttatagaagatgatgacaatggattggaaagtgattttgcaggcccaggagatacaatgcaggttgaacctccccctttggaaataaactccagagtttctttgaaggttggagaaagtacagaatctggaacagtaatattctgtgatgttttaccaggaaaagagagtctaggatattttgttggtgtggacatggataaccctattggcaactgggatggaaggtttgatggagtacagctctgtagttttgcaagtgttgaaagtacaattctcctgcacatcaatgacatcatcccagatagcgtgacacaggaaaggaggcctcccaaacttgcctttatgtcaagaggtgtaggtgacaaaggttcatctagtcataataaaccaaaggttacaggatctacctcagaccctggaagtagaaacagatctgaattattttataccttaaatgggtcatctgttgactcacaacaatccaagtccaaaaatccatggtacattgatgaagttgcagaagaccctgcaaagtcacttacagagatgtcttcggacttcggacattcatctcctccaccgcagcctccttccatgaactccttgtctagcgagaacagattccactccttacccttcagcctgacaaagatgcccaatactaatggcagcatggctcatagtccactctctctgtcagtgcagtctgtgatgggggagctgaacagcacacctgtccaggagagtccacccttgcccatctcttctgggaatgcacacgggctagaggtgggctcactggctgaagtaaaagagaaccccccgttctatggggttatccgttggattggccagccaccagggctcagtgacgtgctagctggactggaactggaagatgaatgcgcaggctgtacagatggaactttcaggggcacgcggtatttcacgtgtgccctgaagaaggcactgtttgtgaaactgaagagctgcagaccggactctaggtttgcatccttgcagcctgtttccaatcaaattgaaaggtgtaactctttagcatttgggggctatttaagtgaagtagtagaagaaaatactccacctaaaatggaaaaggaaggtttagagataatgattggaaagaagaaaggcatccagggccattacaattcttgttacttagactcaactttattctgcttatttgcttttagttctgccctggacactgtgttacttagacccaaagagaagaatgatatagagtattacagtgagactcaggagctactgaggacagagatagtcaatcctctgagaatatatggatatgtgtgtgccacaaagattatgaaactgaggaaaatacttgaaaaagttgaggctgcatcaggttttacctctgaagaaaaagatcctgaagaattcttaaatatcctgtttcatgatattttaagggttgaaccattgttaaaaataagatcagcaggtcaaaaagttcaagactgtaacttctatcaaatttttatggaaaaaaatgagaaagttggagtacccacaattcagcagttattagaatggtcttttatcaacagcaacctgaaatttgcagaggcaccatcatgcttgattatccagatgcctcggtttggaaaagactttaaactatttaaaaaaatttttccttccctggaattaaatataacagatttacttgaagacactcccaggcagtgccgcatctgtggaggacttgcgatgtacgagtgcagggagtgctatgacgatccggacatctcagctgggaagatcaagcagttctgtaagacctgcagcactcaggttcaccttcatcccaggaggttgaatcattcttatcatccagtatcacttcccaaagacttgcctgactgggactggagacacggctgcatcccctgtcagaagatggagttatttgctgttctctgcatagaaacgagccactatgttgcttttgtgaagtatgggaaggatgactctgcctggcttttctttgacagcatggcggatcgagatggtggtcagaatggcttcaacattccacaagtgacgccctgcccagaagtgggagagtacttgaagatgtctctggaggacctgcactctttggattccagaaggattcaaggctgtgcgcgcagacttctttgcgatgcatacatgtgcatgtaccagagtccaaccatgagcctgtacaaataaccaggttatccgcagtgggcagagacagcacaaaccttagtgctgtgtcttcctccagcttgtggttctgtccggcacccattgccggcaatgggtgtctcttttgatgatggcccttcagcaaaggattcctgtgtttccaaacaaattgcttttgtgttcctgaagtatttaataagaagcattttgcactctaggaagtatgtttgtgttgattttttaagaagtctaaatggatttattaatatctgacactttaagttaactgcattgatgatacgatatttttggaagcatacaattttaattgtgacgtctaaaacctcttttagtccattgaggatgtaaataaatatttcttcttatggaccagggatatgaaatcattttccttttgtagctaatgattgccttgagaaagacataatatgattttattaagagtctgcctttactccaattgttgaaggtgtgctgagtttaattgttaattttcctatatagtgctgatttcctgcatgtctgtactttgctgagtttctgtgttcctgcagcagtggtcagaacatgctgaaattcctttcatggtgttattttttatttgttctagttttgagataaatgagtgactctgtcataaaatgtccatttttgatttgttcttgaagaattagggtacaaaacaagtatcgtagtcatagtaacaactgttactgaacaggtcttctgtaggggctcctaagtatgagacaacatgcagtggtttctgaaatgactgagcgataagtaaattatatattctttttttaaaagatttattttatttatatgagtacactgtagctgtcttcagacacaccagaagagggcatcagatcttattacagatggttgtgagccaccatgtggttgctgggaattgaactcaggacctttggaagggcagtcagtgctcttaactgctgagctatctctctagccccaaattatatattcttatacagacccacagtccagagaaagtgaagctggctcagagttagcaccaacatttccctgaaaaaatgtgttaggtgtttaaaaatctcatcgtaaataaattattttatttttagtttaagtgaatttctcttaatttaaataagactaatatttttgtttgactgaagacccttgtactctctggtgtcagtttggtgtcacttgaaacaatgacaaccacgtcagcatgttactgcaatgtcagtgatggtttcaaaggagaaaatggatgtcatcttaggactgcttgtgtgtgtatagaccgtggtggatgctgtagagtgcttgttgtctgagtgttgttttgtggtatggctgtctctctgaataatgtcaaccttacagaaagagaaggtgttatatgtgtaacatatggctttataaaagatttaatgtcaaaagagtctataaatattgctgttatttttaaaggcactttactccttggttttattattccattttgctggcgtgtattagctttatataaatcacaacacacaaaactcgtccatcttgtaatctttgtacttttgcaatggaaaagtgtgtatactgtgcacagacttttatatagctgagtgcctagactgggtggagaggccatgagtagatagcagtaaagtcctccagctataggctagccttgtcttcatcatcctgggacaggtgagaacggtgagcacaggtgagtctgagcttgcacactgaccagaattcagtcaccctgtaataagtatgtatttaatgagtctgttagtgtggtagtgtgattttgaaaggtttaactaaggaatggtgaggtctttgtttataggcaagcctggcttaacagtgaaggggagggggctgagggaaagctttttttttttttaatttcagaaatggaagcaaaatccatgtgcaaagagactagctacttggctttcagagcagaggactcaaagcttgtttgcagtggttctgaagttggggtggcactttgtgagtgaggaggaccatgtccacaggcaatccctaagcaagatcgtctcgaaattgcactgtgttctttagcagtagtggctgggtaggggcacacagcggaacacccacttgcagtggctggggaggcctgagaagcacaggccagcgggagctctctcccatgccttcttgtccctctggacagcccctcctcattccttgtcccagcagagcctggtttgcccctggggcaatagggttggactgtgtggtgcagatagccctcgtgcctgtggattactttaattttgtaatcttaggttttctgtacggttttcccattgccttttaaaatattttgagtgtccttctccttatcaagatggtagtgtagtaggattgttttactcacaacatggtttaggagccatagctacaaataggaaatataagaattctatttctctactgtgaggcaaatctgtatatatgtatggagagtcaggaggatgaacaagggagaggaaaggggcccaggcacatttttaatccaaaagctacaaagtgtcgagctcctcagggccagcttcacagagagtccagctcagttcacaaagtattttctaagttggggaaatgcattgagaaacagagcgagatatctgtgtagtgcagctgtgggtggttgctatgcattttatcctcagagtagcctggggtttttattttatgtttgccaagtgcttaggtaaatggccgggctcctcttacctctgacttctggagcgtgtccattctctggttgactgaatgtggcattttcataaatctaggagcagcagccctcccaactgcaatttagtaagcaggtagataagcttgcatctgtttgcatcttcctgcactgagttgggagtgcaaaaccccgtacttggcctggtgagtaactgccaacaagtatttatgggtcatttgctaggtggctagcatacggcacggggctaatgagacctggggcctgctctcatatagcttactgctcagtttctataccagagcatccgttggtgcttcttagcctaggcatgggtgacaggcatgtctggggaccactttgggagacaacctagtgtaaccttattgcaaaggactaagagacaaaaatcctgggttctttctccatcacttgctctgtagctgaatgaacaccacctcgatttcttgagtttcagtgtcttgttctgtctgatgaaagggttggatatgtagttggataagcggtacaaattgttagggttccaggccatgcctgatacatctgagtttttgctggaggtaggaaatttcaactcagaagctcatccacagaaaacagtgaccagcagcaggtatggagccataaggctggaaggcttcaaagaccccttcttctgttactgttatccacggcagttattctgtgtaggtgtgaaggagtggctactgactctgtgtggatcagcgtttacatatgacctgatgggagtgcatctggtatgtgttcctcccccctgtattgctggagagattggcaatcgtaggtatacacggaagtgtcgggatgttcagtggcgcgcacttggatgtttcttttcactttatttctatagaaacaggttccaggagccagagaagtaggggaaaaaatccctgagctcatctctctctactacttaataatgcaaacatttaaattcttgggaagattttcccgccccactccctacccccagtcacaggtcagaataggatttccttcttagagaaggtaggtcatggggcctgcgattaaaaaaacaaaaacataaaacaaacaaacaaacaaggtggctttccttggtctttgtgattccagctcttcaccagtgaaggaattctttagttccatagtgcagcctgggtgggtggagaagaggctgttatagtgggagccagtgagcaaagagggttggaggaggcagaggacagggctcagggaaggcaagcctgtggctctgggcattggccctcttgtcaaaggtgtcagagcatggcttcctgtgaggggctttcccttgctcctgtgtgagaatctgtgcgttgtcttactggatgggggacctggatggagatgttaggggagatagcttagtcttgttctaaactctagacttgctaaaaagggttctcttgagtccaaatgcagtgccagagggtgagaagcagaattggccagtagtgaacagtggtattgtgggtgataagccctttctggtggccttaaatagcagtgctagtccttgagtgccacagggtcccctacctccgtcctggcagtgctgatgggtttgtttcagcatattttggttctcattctcatttagggaaaagggctgttgatgaagggattatcctcccaggcaccaggggcctcagcacagttccctgtgcttttaaacactgttgactttcattagttattctaaagggatttatattggagatacaactccctttggtttcatattatgaaatagctttgtactgtggaatgctaactctggaaagtctccctcataccagaaccaaacattcatttcttttatatatatatatatattatctccctggtactctccagggaggtggttattatcaagctgacccaaaggagtgcagtatgtgttctagttccttttctggtaccttgttcaacattcttttttcctgaagaacatccttttgtttatgaaaacgtgaatagcatcatgtaaaacaatcccttgctatcagaatgctccagtttggccttaattttgtaaaccttggagaggagtggagcgcggctgtgagaagagatgtaccatgtactctttctgtatcattaaagtgcagtttttatgcttct

SEQ ID NO: 45 - DNA, 8014 bp, Mus musculus
agttcggaggcggggcaggtgggggcgggcccaggtagcaggttcggctgcgcgggggcccgcgcgcctgaggcactttgaattgctgtctttttacaacatggatgccaggttgctaaaatcttgctttgggacttacagcgagttcatttttttggattttatgacagatttactagtggctcctttttgccagtcaatgttctgaaagttactgtcacaatgagttcaggcctgtggagccaagagaaagttacttcaccctactgggaagaacggattttttatctgcttcttcaagaatgcagtgtaacagacaaacaaactcagaagctgctgaaagtacccaaagggagtataggacagtacatccaagaccgttctgtggggcattcaagagttccttccacaaaaggcaagaaaaatcagattggattaaaaatcttggagcaaccgcatgcagttctgtttgttgatgaaaaggatgttgtagaaataaatgaaaaattcacagagttactgttggcaattaccaactgtgaggagaggctcagcctatttagaaacagactccgactaagtaaaggcctccaggtagacgtgggcagtcctgtgaaagtacagctgcgatctggggaagagaaatttccaggagttgtacgcttcagaggacctttattagcggagaggacggtgtcggggattttctttggagtagaattattggaagaaggtcgtggtcaaggtttcacggatggggtatatcaagggaagcagcttttccagtgtgatgaggactgtggcgtgtttgttgcattggacaaactagaacttatagaagatgatgacaatggattggaaagtgattttgcaggcccaggagatacaatgcaggttgaacctccccctttggaaataaactccagagtttctttgaaggttggagaaagtacagaatctggaacagtaatattctgtgatgttttaccaggaaaagagagtctaggatattttgttggtgtggacatggataaccctattggcaactgggatggaaggtttgatggagtacagctctgtagttttgcaagtgttgaaagtacaattctcctgcacatcaatgacatcatcccagatagcgtgacacaggaaaggaggcctcccaaacttgcctttatgtcaagaggtgtaggtgacaaaggttcatctagtcataataaaccaaaggttacaggatctacctcagaccctggaagtagaaacagatctgaattattttataccttaaatgggtcatctgttgactcacaacaatccaagtccaaaaatccatggtacattgatgaagttgcagaagaccctgcaaagtcacttacagagatgtcttcggacttcggacattcatctcctccaccgcagcctccttccatgaactccttgtctagcgagaacagattccactccttacccttcagcctgacaaagatgcccaatactaatggcagcatggctcatagtccactctctctgtcagtgcagtctgtgatgggggagctgaacagcacacctgtccaggagagtccacccttgcccatctcttctgggaatgcacacgggctagaggtgggctcactggctgaagtaaaagagaaccccccgttctatggggttatccgttggattggccagccaccagggctcagtgacgtgctagctggactggaactggaagatgaatgcgcaggctgtacagatggaactttcaggggcacgcggtatttcacgtgtgccctgaagaaggcactgtttgtgaaactgaagagctgcagaccggactctaggtttgcatccttgcagcctgtttccaatcaaattgaaaggtgtaactctttagcatttgggggctatttaagtgaagtagtagaagaaaatactccacctaaaatggaaaaggaaggtttagagataatgattggaaagaagaaaggcatccagggccattacaattcttgttacttagactcaactttattctgcttatttgcttttagttctgccctggacactgtgttacttagacccaaagagaagaatgatatagagtattacagtgagactcaggagctactgaggacagagatagtcaatcctctgagaatatatggatatgtgtgtgccacaaagattatgaaactgaggaaaatacttgaaaaagttgaggctgcatcaggttttacctctgaagaaaaagatcctgaagaattcttaaatatcctgtttcatgatattttaagggttgaaccattgttaaaaataagatcagcaggtcaaaaagttcaagactgtaacttctatcaaatttttatggaaaaaaatgagaaagttggagtacccacaattcagcagttattagaatggtcttttatcaacagcaacctgaaatttgcagaggcaccatcatgcttgattatccagatgcctcggtttggaaaagactttaaactatttaaaaaaatttttccttccctggaattaaatataacagatttacttgaagacactcccaggcagtgccgcatctgtggaggacttgcgatgtacgagtgcagggagtgctatgacgatccggacatctcagctgggaagatcaagcagttctgtaagacctgcagcactcaggttcaccttcatcccaggaggttgaatcattcttatcatccagtatcacttcccaaagacttgcctgactgggactggagacacggctgcatcccctgtcagaagatggagttatttgctgttctctgcatagaaacgagccactatgttgcttttgtgaagtatgggaaggatgactctgcctggcttttctttgacagcatggcggatcgagatggtggtcagaatggcttcaacattccacaagtgacgccctgcccagaagtgggagagtacttgaagatgtctctggaggacctgcactctttggattccagaaggattcaaggctgtgcgcgcagacttctttgcgatgcatacatgtgcatgtaccagagtccaaccatgagcctgtacaaataaccaggttatccgcagtgggcagagacagcacaaaccttagtgctgtgtcttcctccagcttgtggttctgtccggcacccattgccggcaatgggtgtctcttttgatgatggcccttcagcaaaggattcctgtgtttccaaacaaattgcttttgtgttcctgaagtatttaataagaagcattttgcactctaggaagtatgtttgtgttgattttttaagaagtctaaatggatttattaatatctgacactttaagttaactgcattgatgatacgatatttttggaagcatacaattttaattgtgacgtctaaaacctcttttagtccattgaggatgtaaataaatatttcttcttatggaccagggatatgaaatcattttccttttgtagctaatgattgccttgagaaagacataatatgattttattaagagtctgcctttactccaattgttgaaggtgtgctgagtttaattgttaattttcctatatagtgctgatttcctgcatgtctgtactttgctgagtttctgtgttcctgcagcagtggtcagaacatgctgaaattcctttcatggtgttattttttatttgttctagttttgagataaatgagtgactctgtcataaaatgtccatttttgatttgttcttgaagaattagggtacaaaacaagtatcgtagtcatagtaacaactgttactgaacaggtcttctgtaggggctcctaagtatgagacaacatgcagtggtttctgaaatgactgagcgataagtaaattatatattctttttttaaaagatttattttatttatatgagtacactgtagctgtcttcagacacaccagaagagggcatcagatcttattacagatggttgtgagccaccatgtggttgctgggaattgaactcaggacctttggaagggcagtcagtgctcttaactgctgagctatctctctagccccaaattatatattcttatacagacccacagtccagagaaagtgaagctggctcagagttagcaccaacatttccctgaaaaaatgtgttaggtgtttaaaaatctcatcgtaaataaattattttatttttagtttaagtgaatttctcttaatttaaataagactaatatttttgtttgactgaagacccttgtactctctggtgtcagtttggtgtcacttgaaacaatgacaaccacgtcagcatgttactgcaatgtcagtgatggtttcaaaggagaaaatggatgtcatcttaggactgcttgtgtgtgtatagaccgtggtggatgctgtagagtgcttgttgtctgagtgttgttttgtggtatggctgtctctctgaataatgtcaaccttacagaaagagaaggtgttatatgtgtaacatatggctttataaaagatttaatgtcaaaagagtctataaatattgctgttatttttaaaggcactttactccttggttttattattccattttgctggcgtgtattagctttatataaatcacaacacacaaaactcgtccatcttgtaatctttgtacttttgcaatggaaaagtgtgtatactgtgcacagacttttatatagctgagtgcctagactgggtggagaggccatgagtagatagcagtaaagtcctccagctataggctagccttgtcttcatcatcctgggacaggtgagaacggtgagcacaggtgagtctgagcttgcacactgaccagaattcagtcaccctgtaataagtatgtatttaatgagtctgttagtgtggtagtgtgattttgaaaggtttaactaaggaatggtgaggtctttgtttataggcaagcctggcttaacagtgaaggggagggggctgagggaaagctttttttttttttaatttcagaaatggaagcaaaatccatgtgcaaagagactagctacttggctttcagagcagaggactcaaagcttgtttgcagtggttctgaagttggggtggcactttgtgagtgaggaggaccatgtccacaggcaatccctaagcaagatcgtctcgaaattgcactgtgttctttagcagtagtggctgggtaggggcacacagcggaacacccacttgcagtggctggggaggcctgagaagcacaggccagcgggagctctctcccatgccttcttgtccctctggacagcccctcctcattccttgtcccagcagagcctggtttgcccctggggcaatagggttggactgtgtggtgcagatagccctcgtgcctgtggattactttaattttgtaatcttaggttttctgtacggttttcccattgccttttaaaatattttgagtgtccttctccttatcaagatggtagtgtagtaggattgttttactcacaacatggtttaggagccatagctacaaataggaaatataagaattctatttctctactgtgaggcaaatctgtatatatgtatggagagtcaggaggatgaacaagggagaggaaaggggcccaggcacatttttaatccaaaagctacaaagtgtcgagctcctcagggccagcttcacagagagtccagctcagttcacaaagtattttctaagttggggaaatgcattgagaaacagagcgagatatctgtgtagtgcagctgtgggtggttgctatgcattttatcctcagagtagcctggggtttttattttatgtttgccaagtgcttaggtaaatggccgggctcctcttacctctgacttctggagcgtgtccattctctggttgactgaatgtggcattttcataaatctaggagcagcagccctcccaactgcaatttagtaagcaggtagataagcttgcatctgtttgcatcttcctgcactgagttgggagtgcaaaaccccgtacttggcctggtgagtaactgccaacaagtatttatgggtcatttgctaggtggctagcatacggcacggggctaatgagacctggggcctgctctcatatagcttactgctcagtttctataccagagcatccgttggtgcttcttagcctaggcatgggtgacaggcatgtctggggaccactttgggagacaacctagtgtaaccttattgcaaaggactaagagacaaaaatcctgggttctttctccatcacttgctctgtagctgaatgaacaccacctcgatttcttgagtttcagtgtcttgttctgtctgatgaaagggttggatatgtagttggataagcggtacaaattgttagggttccaggccatgcctgatacatctgagtttttgctggaggtaggaaatttcaactcagaagctcatccacagaaaacagtgaccagcagcaggtatggagccataaggctggaaggcttcaaagaccccttcttctgttactgttatccacggcagttattctgtgtaggtgtgaaggagtggctactgactctgtgtggatcagcgtttacatatgacctgatgggagtgcatctggtatgtgttcctcccccctgtattgctggagagattggcaatcgtaggtatacacggaagtgtcgggatgttcagtggcgcgcacttggatgtttcttttcactttatttctatagaaacaggttccaggagccagagaagtaggggaaaaaatccctgagctcatctctctctactacttaataatgcaaacatttaaattcttgggaagattttcccgccccactccctacccccagtcacaggtcagaataggatttccttcttagagaaggtaggtcatggggcctgcgattaaaaaaacaaaaacataaaacaaacaaacaaacaaggtggctttccttggtctttgtgattccagctcttcaccagtgaaggaattctttagttccatagtgcagcctgggtgggtggagaagaggctgttatagtgggagccagtgagcaaagagggttggaggaggcagaggacagggctcagggaaggcaagcctgtggctctgggcattggccctcttgtcaaaggtgtcagagcatggcttcctgtgaggggctttcccttgctcctgtgtgagaatctgtgcgttgtcttactggatgggggacctggatggagatgttaggggagatagcttagtcttgttctaaactctagacttgctaaaaagggttctcttgagtccaaatgcagtgccagagggtgagaagcagaattggccagtagtgaacagtggtattgtgggtgataagccctttctggtggccttaaatagcagtgctagtccttgagtgccacagggtcccctacctccgtcctggcagtgctgatgggtttgtttcagcatattttggttctcattctcatttagggaaaagggctgttgatgaagggattatcctcccaggcaccaggggcctcagcacagttccctgtgcttttaaacactgttgactttcattagttattctaaagggatttatattggagatacaactccctttggtttcatattatgaaatagctttgtactgtggaatgctaactctggaaagtctccctcataccagaaccaaacattcatttcttttatatatatatatatattatctccctggtactctccagggaggtggttattatcaagctgacccaaaggagtgcagtatgtgttctagttccttttctggtaccttgttcaacattcttttttcctgaagaacatccttttgtttatgaaaacgtgaatagcatcatgtaaaacaatcccttgctatcagaatgctccagtttggccttaattttgtaaaccttggagaggagtggagcgcggctgtgagaagagatgtaccatgtactctttctgtatcattaaagtgcagtttttatgcttct

SEQ ID NO: 46 - protein, 770 aa, Mus musculus
MSSGLWSQEKVTSPYWEERIFYLLLQECSVTDKQTQKLLKVPKGSIGQYIQDRSVGHSRVPSTKGKKNQIGLKILEQPHAVLFVDEKDVVEINEKFTELLLAITNCEERLSLFRNRLRLSKGLQVDVGSPVKVQLRSGEEKFPGVVRFRGPLLAERTVSGIFFGVELLEEGRGQGFTDGVYQGKQLFQCDEDCGVFVALDKLELIEDDDNGLESDFAGPGDTMQVEPPPLEINSRVSLKVGESTESGTVIFCDVLPGKESLGYFVGVDMDNPIGNWDGRFDGVQLCSFASVESTILLHINDIIPDSVTQERRPPKLAFMSRGVGDKGSSSHNKPKVTGSTSDPGSRNRSELFYTLNGSSVDSQQSKSKNPWYIDEAFGGYLSEVVEENTPPKMEKEGLEIMIGKKKGIQGHYNSCYLDSTLFCLFAFSSALDTVLLRPKEKNDIEYYSETQELLRTEIVNPLRIYGYVCATKIMKLRKILEKVEAASGFTSEEKDPEEFLNILFHDILRVEPLLKIRSAGQKVQDCNFYQIFMEKNEKVGVPTIQQLLEWSFINSNLKFAEAPSCLIIQMPRFGKDFKLFKKIFPSLELNITDLLEDTPRQCRICGGLAMYECRECYDDPDISAGKIKQFCKTCSTQVHLHPRRLNHSYHPVSLPKDLPDWDWRHGCIPCQKMELFAVLCIETSHYVAFVKYGKDDSAWLFFDSMADRDGGQNGFNIPQVTPCPEVGEYLKMSLEDLHSLDSRRIQGCARRLLCDAYMCMYQSPTMSLYK

SEQ ID NO: 47 - DNA, 7599 bp, Mus musculus
agttcggaggcggggcaggtgggggcgggcccaggtagcaggttcggctgcgcgggggcccgcgcgcctgagtgagctcctatgtcatggattctgaactggcttataatatctggcatggattccttttagcagagcaggcctcaaatccgatagagcatggttgcttgatcccattgcagccctgccactagtggctcctggcactttgaattgctgtctttttacaacatggatgccaggttgctaaaatcttgctttgggacttacagcgagttcatttttttggattttatgacagatttactagtggctcctttttgccagtcaatgttctgaaagttactgtcacaatgagttcaggcctgtggagccaagagaaagttacttcaccctactgggaagaacggattttttatctgcttcttcaagaatgcagtgtaacagacaaacaaactcagaagctgctgaaagtacccaaagggagtataggacagtacatccaagaccgttctgtggggcattcaagagttccttccacaaaaggcaagaaaaatcagattggattaaaaatcttggagcaaccgcatgcagttctgtttgttgatgaaaaggatgttgtagaaataaatgaaaaattcacagagttactgttggcaattaccaactgtgaggagaggctcagcctatttagaaacagactccgactaagtaaaggcctccaggtagacgtgggcagtcctgtgaaagtacagctgcgatctggggaagagaaatttccaggagttgtacgcttcagaggacctttattagcggagaggacggtgtcggggattttctttggagtagaattattggaagaaggtcgtggtcaaggtttcacggatggggtatatcaagggaagcagcttttccagtgtgatgaggactgtggcgtgtttgttgcattggacaaactagaacttatagaagatgatgacaatggattggaaagtgattttgcaggcccaggagatacaatgcaggttgaacctccccctttggaaataaactccagagtttctttgaaggttggagaaagtacagaatctggaacagtaatattctgtgatgttttaccaggaaaagagagtctaggatattttgttggtgtggacatggataaccctattggcaactgggatggaaggtttgatggagtacagctctgtagttttgcaagtgttgaaagtacaattctcctgcacatcaatgacatcatcccagatagcgtgacacaggaaaggaggcctcccaaacttgcctttatgtcaagaggtgtaggtgacaaaggttcatctagtcataataaaccaaaggttacaggatctacctcagaccctggaagtagaaacagatctgaattattttataccttaaatgggtcatctgttgactcacaacaatccaagtccaaaaatccatggtacattgatgaagcatttgggggctatttaagtgaagtagtagaagaaaatactccacctaaaatggaaaaggaaggtttagagataatgattggaaagaagaaaggcatccagggccattacaattcttgttacttagactcaactttattctgcttatttgcttttagttctgccctggacactgtgttacttagacccaaagagaagaatgatatagagtattacagtgagactcaggagctactgaggacagagatagtcaatcctctgagaatatatggatatgtgtgtgccacaaagattatgaaactgaggaaaatacttgaaaaagttgaggctgcatcaggttttacctctgaagaaaaagatcctgaagaattcttaaatatcctgtttcatgatattttaagggttgaaccattgttaaaaataagatcagcaggtcaaaaagttcaagactgtaacttctatcaaatttttatggaaaaaaatgagaaagttggagtacccacaattcagcagttattagaatggtcttttatcaacagcaacctgaaatttgcagaggcaccatcatgcttgattatccagatgcctcggtttggaaaagactttaaactatttaaaaaaatttttccttccctggaattaaatataacagatttacttgaagacactcccaggcagtgccgcatctgtggaggacttgcgatgtacgagtgcagggagtgctatgacgatccggacatctcagctgggaagatcaagcagttctgtaagacctgcagcactcaggttcaccttcatcccaggaggttgaatcattcttatcatccagtatcacttcccaaagacttgcctgactgggactggagacacggctgcatcccctgtcagaagatggagttatttgctgttctctgcatagaaacgagccactatgttgcttttgtgaagtatgggaaggatgactctgcctggcttttctttgacagcatggcggatcgagatggtggtcagaatggcttcaacattccacaagtgacgccctgcccagaagtgggagagtacttgaagatgtctctggaggacctgcactctttggattccagaaggattcaaggctgtgcgcgcagacttctttgcgatgcatacatgtgcatgtaccagagtccaaccatgagcctgtacaaataaccaggttatccgcagtgggcagagacagcacaaaccttagtgctgtgtcttcctccagcttgtggttctgtccggcacccattgccggcaatgggtgtctcttttgatgatggcccttcagcaaaggattcctgtgtttccaaacaaattgcttttgtgttcctgaagtatttaataagaagcattttgcactctaggaagtatgtttgtgttgattttttaagaagtctaaatggatttattaatatctgacactttaagttaactgcattgatgatacgatatttttggaagcatacaattttaattgtgacgtctaaaacctcttttagtccattgaggatgtaaataaatatttcttcttatggaccagggatatgaaatcattttccttttgtagctaatgattgccttgagaaagacataatatgattttattaagagtctgcctttactccaattgttgaaggtgtgctgagtttaattgttaattttcctatatagtgctgatttcctgcatgtctgtactttgctgagtttctgtgttcctgcagcagtggtcagaacatgctgaaattcctttcatggtgttattttttatttgttctagttttgagataaatgagtgactctgtcataaaatgtccatttttgatttgttcttgaagaattagggtacaaaacaagtatcgtagtcatagtaacaactgttactgaacaggtcttctgtaggggctcctaagtatgagacaacatgcagtggtttctgaaatgactgagcgataagtaaattatatattctttttttaaaagatttattttatttatatgagtacactgtagctgtcttcagacacaccagaagagggcatcagatcttattacagatggttgtgagccaccatgtggttgctgggaattgaactcaggacctttggaagggcagtcagtgctcttaactgctgagctatctctctagccccaaattatatattcttatacagacccacagtccagagaaagtgaagctggctcagagttagcaccaacatttccctgaaaaaatgtgttaggtgtttaaaaatctcatcgtaaataaattattttatttttagtttaagtgaatttctcttaatttaaataagactaatatttttgtttgactgaagacccttgtactctctggtgtcagtttggtgtcacttgaaacaatgacaaccacgtcagcatgttactgcaatgtcagtgatggtttcaaaggagaaaatggatgtcatcttaggactgcttgtgtgtgtatagaccgtggtggatgctgtagagtgcttgttgtctgagtgttgttttgtggtatggctgtctctctgaataatgtcaaccttacagaaagagaaggtgttatatgtgtaacatatggctttataaaagatttaatgtcaaaagagtctataaatattgctgttatttttaaaggcactttactccttggttttattattccattttgctggcgtgtattagctttatataaatcacaacacacaaaactcgtccatcttgtaatctttgtacttttgcaatggaaaagtgtgtatactgtgcacagacttttatatagctgagtgcctagactgggtggagaggccatgagtagatagcagtaaagtcctccagctataggctagccttgtcttcatcatcctgggacaggtgagaacggtgagcacaggtgagtctgagcttgcacactgaccagaattcagtcaccctgtaataagtatgtatttaatgagtctgttagtgtggtagtgtgattttgaaaggtttaactaaggaatggtgaggtctttgtttataggcaagcctggcttaacagtgaaggggagggggctgagggaaagctttttttttttttaatttcagaaatggaagcaaaatccatgtgcaaagagactagctacttggctttcagagcagaggactcaaagcttgtttgcagtggttctgaagttggggtggcactttgtgagtgaggaggaccatgtccacaggcaatccctaagcaagatcgtctcgaaattgcactgtgttctttagcagtagtggctgggtaggggcacacagcggaacacccacttgcagtggctggggaggcctgagaagcacaggccagcgggagctctctcccatgccttcttgtccctctggacagcccctcctcattccttgtcccagcagagcctggtttgcccctggggcaatagggttggactgtgtggtgcagatagccctcgtgcctgtggattactttaattttgtaatcttaggttttctgtacggttttcccattgccttttaaaatattttgagtgtccttctccttatcaagatggtagtgtagtaggattgttttactcacaacatggtttaggagccatagctacaaataggaaatataagaattctatttctctactgtgaggcaaatctgtatatatgtatggagagtcaggaggatgaacaagggagaggaaaggggcccaggcacatttttaatccaaaagctacaaagtgtcgagctcctcagggccagcttcacagagagtccagctcagttcacaaagtattttctaagttggggaaatgcattgagaaacagagcgagatatctgtgtagtgcagctgtgggtggttgctatgcattttatcctcagagtagcctggggtttttattttatgtttgccaagtgcttaggtaaatggccgggctcctcttacctctgacttctggagcgtgtccattctctggttgactgaatgtggcattttcataaatctaggagcagcagccctcccaactgcaatttagtaagcaggtagataagcttgcatctgtttgcatcttcctgcactgagttgggagtgcaaaaccccgtacttggcctggtgagtaactgccaacaagtatttatgggtcatttgctaggtggctagcatacggcacggggctaatgagacctggggcctgctctcatatagcttactgctcagtttctataccagagcatccgttggtgcttcttagcctaggcatgggtgacaggcatgtctggggaccactttgggagacaacctagtgtaaccttattgcaaaggactaagagacaaaaatcctgggttctttctccatcacttgctctgtagctgaatgaacaccacctcgatttcttgagtttcagtgtcttgttctgtctgatgaaagggttggatatgtagttggataagcggtacaaattgttagggttccaggccatgcctgatacatctgagtttttgctggaggtaggaaatttcaactcagaagctcatccacagaaaacagtgaccagcagcaggtatggagccataaggctggaaggcttcaaagaccccttcttctgttactgttatccacggcagttattctgtgtaggtgtgaaggagtggctactgactctgtgtggatcagcgtttacatatgacctgatgggagtgcatctggtatgtgttcctcccccctgtattgctggagagattggcaatcgtaggtatacacggaagtgtcgggatgttcagtggcgcgcacttggatgtttcttttcactttatttctatagaaacaggttccaggagccagagaagtaggggaaaaaatccctgagctcatctctctctactacttaataatgcaaacatttaaattcttgggaagattttcccgccccactccctacccccagtcacaggtcagaataggatttccttcttagagaaggtaggtcatggggcctgcgattaaaaaaacaaaaacataaaacaaacaaacaaacaaggtggctttccttggtctttgtgattccagctcttcaccagtgaaggaattctttagttccatagtgcagcctgggtgggtggagaagaggctgttatagtgggagccagtgagcaaagagggttggaggaggcagaggacagggctcagggaaggcaagcctgtggctctgggcattggccctcttgtcaaaggtgtcagagcatggcttcctgtgaggggctttcccttgctcctgtgtgagaatctgtgcgttgtcttactggatgggggacctggatggagatgttaggggagatagcttagtcttgttctaaactctagacttgctaaaaagggttctcttgagtccaaatgcagtgccagagggtgagaagcagaattggccagtagtgaacagtggtattgtgggtgataagccctttctggtggccttaaatagcagtgctagtccttgagtgccacagggtcccctacctccgtcctggcagtgctgatgggtttgtttcagcatattttggttctcattctcatttagggaaaagggctgttgatgaagggattatcctcccaggcaccaggggcctcagcacagttccctgtgcttttaaacactgttgactttcattagttattctaaagggatttatattggagatacaactccctttggtttcatattatgaaatagctttgtactgtggaatgctaactctggaaagtctccctcataccagaaccaaacattcatttcttttatatatatatatatattatctccctggtactctccagggaggtggttattatcaagctgacccaaaggagtgcagtatgtgttctagttccttttctggtaccttgttcaacattcttttttcctgaagaacatccttttgtttatgaaaacgtgaatagcatcatgtaaaacaatcccttgctatcagaatgctccagtttggccttaattttgtaaaccttggagaggagtggagcgcggctgtgagaagagatgtaccatgtactctttctgtatcattaaagtgcagtttttatgcttct

SEQ ID NO: 48 - protein, 920 aa, Homo sapiens
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 49 - DNA, 10667 bp, Homo sapiens
agcgccccctccgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaacaaaaaaaaaaagcaaaaacaaaacaaaaaataagccaaaaaaccttgctagtgttttttcctcaaaaataaataaataaataaataaatacgtacatacatacacacatacatacaaacatatagaaatccccaaagaggccaatagtgacgagaaggtgaaaattgcaggcccatggggagttactgattttttcatctcctccctccacgggagactttattttctgccaatggctattgccattagagggcagagtgaccccagagctgagttgggcaggggggtggacagagaggagaggacaaggagggcaatggagcatcagtacctgcccacagccttggtccctgggggctagactgctcaactgtggagcaattcattatactgaaaatgtgcttgttgttgaaaatttgtctgcatgttaatgcctcacccccaaacccttttctctctcactctctgcctccaacttcagattgactttcaatagtttttctaagacctttgaactgaatgttctcttcagccaaaacttggcgacttccacagaaaagtctgaccactgagaagaaggagagcagagatttaaccctttgtaaggccccatttggatccaggtctgctttctcatgtgtgagtcagggaggagctggagccagaggagaagaaaatgatagcttggctgttctcctgcttaggacactgactgaatagttaaactctcactgccactaccttttccccacctttaaaagacctgaatgaagttttctgccaaactccgtgaagccacaagcaccttatgtcctcccttcagtgttttgtgggcctgaatttcatcacactgcatttcagccatggtcatcaagcctgtttgcttcttttgggcatgttcacagattctctgttaagagcccccaccaccaagaaggttagcaggccaacagctctgacatctatctgtagatgccagtagtcacaaagatttcttaccaactctcagatcgctggagcccttagacaaactggaaagaaggcatcaaagggatcaggcaagctgggcgtcttgcccttgtcccccagagatgataccctcccagcaagtggagaagttctcacttccttctttagagcagctaaaggggctacccagatcagggttgaagagaaaactcaattaccagggtgggaagaatgaaggcactagaaccagaaaccctgcaaatgctcttcttgtcacccagcatatccacctgcagaagtcatgagaagagagaaggaacaaagaggagactctgactactgaattaaaatcttcagcggcaaagcctaaagccagatggacaccatctggtgagtttactcatcatcctcctctgctgctgattctgggctctgacattgcccatactcactcagattccccacctttgttgctgcctcttagtcagagggaggccaaaccattgagactttctacagaaccatggcttctttcggaaaggtctggttggtgtggctccaatactttgccacccatgaactcagggtgtgccctgggacactggttttatatagtcttttggcacacctgtgttctgttgacttcgttcttcaagcccaagtgcaagggaaaatgtccacctactttctcatcttggcctctgcctccttacttagctcttaatctcatctgttgaactcaagaaatcaagggccagtcatcaagctgcccattttaattgattcactctgtttgttgagaggatagtttctgagtgacatgatatgatccacaagggtttccttccctgatttctgcattgatattaatagccaaacgaacttcaaaacagctttaaataacaagggagaggggaacctaagatgagtaatatgccaatccaagactgctggagaaaactaaagctgacaggttccctttttggggtgggatagacatgttctggttttctttattattacacaatctggctcatgtacaggatcacttttagctgttttaaacagaaaaaaatatccaccactcttttcagttacactaggttacattttaataggtcctttacatctgttttggaatgattttcatcttttgtgatacacagattgaattatatcattttcatatctctccttgtaaatactagaagctctcctttacatttctctatcaaatttttcatctttatgggtttcccaattgtgactcttgtcttcatgaatatatgtttttcatttgcaaaagccaaaaatcagtgaaacagcagtgtaattaaaagcaacaactggattactccaaatttccaaatgacaaaactagggaaaaatagcctacacaagcctttaggcctactctttctgtgcttgggtttgagtgaacaaaggagattttagcttggctctgttctcccatggatgaaaggaggaggattttttttttcttttggccattgatgttctagccaatgtaattgacagaagtctcattttgcatgcgctctgctctacaaacagagttggtatggttggtatactgtactcacctgtgagggactggccactcagacccacttagctggtgagctagaagatgaggatcactcactggaaaagtcacaaggaccatctccaaacaagttggcagtgctcgatgtggacgaagagtgaggaagagaaaaagaaggagcaccagggagaaggctccgtctgtgctgggcagcagacagctgccaggatcacgaactctgtagtcaaagaaaagagtcgtgtggcagtttcagctctcgttcattgggcagctcgcctaggcccagcctctgagctgacatgggagttgttggattctttgtttcatagctttttctatgccataggcaatattgttgttcttggaaagtttattatttttttaactcccttactctgagaaagggatattttgaaggactgtcatatatctttgaaaaaagaaaatctgtaatacatatatttttatgtatgttcactggcactaaaaaatatagagagcttcattctgtcctttgggtagttgctgaggtaattgtccaggttgaaaaataatgtgctgatgctagagtccctctctgtccatactctacttctaaatacatataggcatacatagcaagttttatttgacttgtactttaagagaaaatatgtccaccatccacatgatgcacaaatgagctaacattgagcttcaagtagcttctaagtgtttgtttcattaggcacagcacagatgtggcctttccccccttctctcccttgatatctggcagggcataaaggcccaggccacttcctctgccccttcccagccctgcaccaaagctgcatttcaggagactctctccagacagcccagtaactacccgagcatggcccctgcatagccctggaaaaataagaggctgactgtctacgaattatcttgtgccagttgcccaggtgagagggcactgggccaagggagtggttttcatgtttgacccactacaaggggtcatgggaatcaggaatgccaaagcaccagatcaaatccaaaacttaaagtcaaaataagccattcagcatgttcagtttcttggaaaaggaagtttctacccctgatgcctttgtaggcagatctgttctcaccattaatctttttgaaaatcttttaaagcagtttttaaaaagagagatgaaagcatcacattatataaccaaagattacattgtacctgctaagataccaaaattcataagggcagggggggagcaagcattagtgcctctttgataagctgtccaaagacagactaaaggactctgctggtgactgacttataagagctttgtgggtttttttttccctaataatatacatgtttagaagaattgaaaataatttcgggaaaatgggattatgggtccttcactaagtgattttataagcagaactggctttccttttctctagtagttgctgagcaaattgttgaagctccatcattgcatggttggaaatggagctgttcttagccactgtgtttgctagtgcccatgttagcttatctgaagatgtgaaacccttgctgataagggagcatttaaagtactagattttgcactagagggacagcaggcagaaatccttatttctgcccactttggatggcacaaaaagttatctgcagttgaaggcagaaagttgaaatacattgtaaatgaatatttgtatccatgtttcaaaattgaaatatatatatatatatatatatatatatatatatatatatagtgtgtgtgtgtgttctgatagctttaactttctctgcatctttatatttggttccagatcacacctgatgccatgtacttgtgagagaggatgcagttttgttttggaagctctctcagaacaaacaagacacctggattgatcagttaactaaaagttttctcccctattgggtttgacccacaggtcctgtgaaggagcagagggataaaaagagtagaggacatgatacattgtactttactagttcaagacagatgaatgtggaaagcataaaaactcaatggaactgactgagatttaccacagggaaggcccaaacttggggccaaaagcctacccaagtgattgaccagtggccccctaatgggacctgagctgttggaagaagagaactgttccttggtcttcaccatccttgtgagagaagggcagtttcctgcattggaacctggagcaagcgctctatctttcacacaaattccctcacctgagattgaggtgctcttgttactgggtgtctgtgtgctgtaattctggttttggatatgttctgtaaagattttgacaaatgaaaatgtgtttttctctgttaaaacttgtcagagtactagaagttgtatctctgtaggtgcaggtccatttctgcccacaggtagggtgtttttctttgattaagagattgacacttctgttgcctaggacctcccaactcaaccatttctaggtgaaggcagaaaaatccacattagttactcctcttcagacatttcagctgagataacaaatcttttggaattttttcacccatagaaagagtggtagatatttgaatttagcaggtggagtttcatagtaaaaacagcttttgactcagctttgatttatcctcatttgatttggccagaaagtaggtaatatgcattgattggcttctgattccaattcagtatagcaaggtgctaggttttttcctttccccacctgtctcttagcctggggaattaaatgagaagccttagaatgggtggcccttgtgacctgaaacacttcccacataagctacttaacaagattgtcatggagctgcagattccattgcccaccaaagactagaacacacacatatccatacaccaaaggaaagacaattctgaaatgctgtttctctggtggttccctctctggctgctgcctcacagtatgggaacctgtactctgcagaggtgacaggccagatttgcattatctcacaaccttagcccttggtgctaactgtcctacagtgaagtgcctggggggttgtcctatcccataagccacttggatgctgacagcagccaccatcagaatgacccacgcaaaaaaaagaaaaaaaaaattaaaaagtcccctcacaacccagtgacacctttctgctttcctctagactggaacattgattagggagtgcctcagacatgacattcttgtgctgtccttggaattaatctggcagcaggagggagcagactatgtaaacagagataaaaattaattttcaatattgaaggaaaaaagaaataagaagagagagagaaagaaagcatcacacaaagattttcttaaaagaaacaattttgcttgaaatctctttagatggggctcatttctcacggtggcacttggcctccactgggcagcaggaccagctccaagcgctagtgttctgttctctttttgtaatcttggaatcttttgttgctctaaatacaattaaaaatggcagaaacttgtttgttggactacatgtgtgactttgggtctgtctctgcctctgctttcagaaatgtcatccattgtgtaaaatattggcttactggtctgccagctaaaacttggccacatcccctgttatggctgcaggatcgagttattgttaacaaagagacccaagaaaagctgctaatgtcctcttatcattgttgttaatttgttaaaacataaagaaatctaaaatttca

SEQ ID NO: 50 - protein, 388 aa, Homo sapiens
MILWLHSLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 51 - DNA, 10257 bp, Homo sapiens
gcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtaggtgctgcgagcagagagggattcctcggaggtcatctgttccatcttcttgcctatgcaaatgcctgcctgaagctgctggaggctggctttgtaccggactttgtacagggaaccagggaaacgaatgcagagtgctcctgacattgcctgtcactttttcccatgatactctggcttcacagtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaacaaaaaaaaaaagcaaaaacaaaacaaaaaataagccaaaaaaccttgctagtgttttttcctcaaaaataaataaataaataaataaatacgtacatacatacacacatacatacaaacatatagaaatccccaaagaggccaatagtgacgagaaggtgaaaattgcaggcccatggggagttactgattttttcatctcctccctccacgggagactttattttctgccaatggctattgccattagagggcagagtgaccccagagctgagttgggcaggggggtggacagagaggagaggacaaggagggcaatggagcatcagtacctgcccacagccttggtccctgggggctagactgctcaactgtggagcaattcattatactgaaaatgtgcttgttgttgaaaatttgtctgcatgttaatgcctcacccccaaacccttttctctctcactctctgcctccaacttcagattgactttcaatagtttttctaagacctttgaactgaatgttctcttcagccaaaacttggcgacttccacagaaaagtctgaccactgagaagaaggagagcagagatttaaccctttgtaaggccccatttggatccaggtctgctttctcatgtgtgagtcagggaggagctggagccagaggagaagaaaatgatagcttggctgttctcctgcttaggacactgactgaatagttaaactctcactgccactaccttttccccacctttaaaagacctgaatgaagttttctgccaaactccgtgaagccacaagcaccttatgtcctcccttcagtgttttgtgggcctgaatttcatcacactgcatttcagccatggtcatcaagcctgtttgcttcttttgggcatgttcacagattctctgttaagagcccccaccaccaagaaggttagcaggccaacagctctgacatctatctgtagatgccagtagtcacaaagatttcttaccaactctcagatcgctggagcccttagacaaactggaaagaaggcatcaaagggatcaggcaagctgggcgtcttgcccttgtcccccagagatgataccctcccagcaagtggagaagttctcacttccttctttagagcagctaaaggggctacccagatcagggttgaagagaaaactcaattaccagggtgggaagaatgaaggcactagaaccagaaaccctgcaaatgctcttcttgtcacccagcatatccacctgcagaagtcatgagaagagagaaggaacaaagaggagactctgactactgaattaaaatcttcagcggcaaagcctaaagccagatggacaccatctggtgagtttactcatcatcctcctctgctgctgattctgggctctgacattgcccatactcactcagattccccacctttgttgctgcctcttagtcagagggaggccaaaccattgagactttctacagaaccatggcttctttcggaaaggtctggttggtgtggctccaatactttgccacccatgaactcagggtgtgccctgggacactggttttatatagtcttttggcacacctgtgttctgttgacttcgttcttcaagcccaagtgcaagggaaaatgtccacctactttctcatcttggcctctgcctccttacttagctcttaatctcatctgttgaactcaagaaatcaagggccagtcatcaagctgcccattttaattgattcactctgtttgttgagaggatagtttctgagtgacatgatatgatccacaagggtttccttccctgatttctgcattgatattaatagccaaacgaacttcaaaacagctttaaataacaagggagaggggaacctaagatgagtaatatgccaatccaagactgctggagaaaactaaagctgacaggttccctttttggggtgggatagacatgttctggttttctttattattacacaatctggctcatgtacaggatcacttttagctgttttaaacagaaaaaaatatccaccactcttttcagttacactaggttacattttaataggtcctttacatctgttttggaatgattttcatcttttgtgatacacagattgaattatatcattttcatatctctccttgtaaatactagaagctctcctttacatttctctatcaaatttttcatctttatgggtttcccaattgtgactcttgtcttcatgaatatatgtttttcatttgcaaaagccaaaaatcagtgaaacagcagtgtaattaaaagcaacaactggattactccaaatttccaaatgacaaaactagggaaaaatagcctacacaagcctttaggcctactctttctgtgcttgggtttgagtgaacaaaggagattttagcttggctctgttctcccatggatgaaaggaggaggattttttttttcttttggccattgatgttctagccaatgtaattgacagaagtctcattttgcatgcgctctgctctacaaacagagttggtatggttggtatactgtactcacctgtgagggactggccactcagacccacttagctggtgagctagaagatgaggatcactcactggaaaagtcacaaggaccatctccaaacaagttggcagtgctcgatgtggacgaagagtgaggaagagaaaaagaaggagcaccagggagaaggctccgtctgtgctgggcagcagacagctgccaggatcacgaactctgtagtcaaagaaaagagtcgtgtggcagtttcagctctcgttcattgggcagctcgcctaggcccagcctctgagctgacatgggagttgttggattctttgtttcatagctttttctatgccataggcaatattgttgttcttggaaagtttattatttttttaactcccttactctgagaaagggatattttgaaggactgtcatatatctttgaaaaaagaaaatctgtaatacatatatttttatgtatgttcactggcactaaaaaatatagagagcttcattctgtcctttgggtagttgctgaggtaattgtccaggttgaaaaataatgtgctgatgctagagtccctctctgtccatactctacttctaaatacatataggcatacatagcaagttttatttgacttgtactttaagagaaaatatgtccaccatccacatgatgcacaaatgagctaacattgagcttcaagtagcttctaagtgtttgtttcattaggcacagcacagatgtggcctttccccccttctctcccttgatatctggcagggcataaaggcccaggccacttcctctgccccttcccagccctgcaccaaagctgcatttcaggagactctctccagacagcccagtaactacccgagcatggcccctgcatagccctggaaaaataagaggctgactgtctacgaattatcttgtgccagttgcccaggtgagagggcactgggccaagggagtggttttcatgtttgacccactacaaggggtcatgggaatcaggaatgccaaagcaccagatcaaatccaaaacttaaagtcaaaataagccattcagcatgttcagtttcttggaaaaggaagtttctacccctgatgcctttgtaggcagatctgttctcaccattaatctttttgaaaatcttttaaagcagtttttaaaaagagagatgaaagcatcacattatataaccaaagattacattgtacctgctaagataccaaaattcataagggcagggggggagcaagcattagtgcctctttgataagctgtccaaagacagactaaaggactctgctggtgactgacttataagagctttgtgggtttttttttccctaataatatacatgtttagaagaattgaaaataatttcgggaaaatgggattatgggtccttcactaagtgattttataagcagaactggctttccttttctctagtagttgctgagcaaattgttgaagctccatcattgcatggttggaaatggagctgttcttagccactgtgtttgctagtgcccatgttagcttatctgaagatgtgaaacccttgctgataagggagcatttaaagtactagattttgcactagagggacagcaggcagaaatccttatttctgcccactttggatggcacaaaaagttatctgcagttgaaggcagaaagttgaaatacattgtaaatgaatatttgtatccatgtttcaaaattgaaatatatatatatatatatatatatatatatatatatatatagtgtgtgtgtgtgttctgatagctttaactttctctgcatctttatatttggttccagatcacacctgatgccatgtacttgtgagagaggatgcagttttgttttggaagctctctcagaacaaacaagacacctggattgatcagttaactaaaagttttctcccctattgggtttgacccacaggtcctgtgaaggagcagagggataaaaagagtagaggacatgatacattgtactttactagttcaagacagatgaatgtggaaagcataaaaactcaatggaactgactgagatttaccacagggaaggcccaaacttggggccaaaagcctacccaagtgattgaccagtggccccctaatgggacctgagctgttggaagaagagaactgttccttggtcttcaccatccttgtgagagaagggcagtttcctgcattggaacctggagcaagcgctctatctttcacacaaattccctcacctgagattgaggtgctcttgttactgggtgtctgtgtgctgtaattctggttttggatatgttctgtaaagattttgacaaatgaaaatgtgtttttctctgttaaaacttgtcagagtactagaagttgtatctctgtaggtgcaggtccatttctgcccacaggtagggtgtttttctttgattaagagattgacacttctgttgcctaggacctcccaactcaaccatttctaggtgaaggcagaaaaatccacattagttactcctcttcagacatttcagctgagataacaaatcttttggaattttttcacccatagaaagagtggtagatatttgaatttagcaggtggagtttcatagtaaaaacagcttttgactcagctttgatttatcctcatttgatttggccagaaagtaggtaatatgcattgattggcttctgattccaattcagtatagcaaggtgctaggttttttcctttccccacctgtctcttagcctggggaattaaatgagaagccttagaatgggtggcccttgtgacctgaaacacttcccacataagctacttaacaagattgtcatggagctgcagattccattgcccaccaaagactagaacacacacatatccatacaccaaaggaaagacaattctgaaatgctgtttctctggtggttccctctctggctgctgcctcacagtatgggaacctgtactctgcagaggtgacaggccagatttgcattatctcacaaccttagcccttggtgctaactgtcctacagtgaagtgcctggggggttgtcctatcccataagccacttggatgctgacagcagccaccatcagaatgacccacgcaaaaaaaagaaaaaaaaaattaaaaagtcccctcacaacccagtgacacctttctgctttcctctagactggaacattgattagggagtgcctcagacatgacattcttgtgctgtccttggaattaatctggcagcaggagggagcagactatgtaaacagagataaaaattaattttcaatattgaaggaaaaaagaaataagaagagagagagaaagaaagcatcacacaaagattttcttaaaagaaacaattttgcttgaaatctctttagatggggctcatttctcacggtggcacttggcctccactgggcagcaggaccagctccaagcgctagtgttctgttctctttttgtaatcttggaatcttttgttgctctaaatacaattaaaaatggcagaaacttgtttgttggactacatgtgtgactttgggtctgtctctgcctctgctttcagaaatgtcatccattgtgtaaaatattggcttactggtctgccagctaaaacttggccacatcccctgttatggctgcaggatcgagttattgttaacaaagagacccaagaaaagctgctaatgtcctcttatcattgttgttaatttgttaaaacataaagaaatctaaaatttcaaaaaa

SEQ ID NO: 52 - protein, 644 aa, Homo sapiens
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGEKFRVGNCKHLKMTRP

SEQ ID NO: 53 - DNA, 3812 bp, Homo sapiens
gcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagaaaaattccgggttggcaattgcaagcatctcaaaatgaccagaccctgaagaaaggctgacttgcctcattcaaaatgagggctctagagggctctagtggatagtctggagaaacctggcgtctgaggcttaggagcttaggtttttgctcctcaacacagactttgacgttggggttgggggctactctcttgattgctgactccctccagcgggaccaatagtgttttcctacctcacagggatgttgtgaggacgggctgtagaagtaatagtggttaccattcatgtagttgtgagtatcatgattattgtttcctgtaatgtggcttggcattggcaaagtgctttttgattgttcttgatcacatatgatgggggccaggcactgactcaggcggatgcagtgaagctctggctcagtcgcttgcttttcgtggtgtgctgccaggaagaaactttgctgatgggactcaaggtgtcaccttggacaagaagcaactgtgtctgtctgaggttcctgtggccatctttatttgtgtattaggcaattcgtatttcccccttaggttctagccttctggatcccagccagtgacctagatcttagcctcaggccctgtcactgagctgaaggtagtagctgatccacagaagttcagtaaacaaggaccagatttctgcttctccaggagaagaagccagccaacccctctcttcaaacacactgagagactacagtccgactttccctcttacatctagccttactgtagccacactccttgattgctctctcacatcacatgcttctcttcatcagttgtaagcctctcattcttctcccaagccagactcaaatattgtattgatgtcaaagaagaatcacttagagtttggaatatcttgttctctctctgctccatagcttccatattgacaccagtttctttctagtggagaagtggagtctgtgaagccagggaaacacacatgtgagagtcagaaggactctccctgacttgcctggggcctgtctttcccaccttctccagtctgtctaaacacacacacacacacacacacacacacacacacgctctctctctctctccccccccaacacacacacactctctctctctctcacacacacacacatacacacacacttctttctctttcccctgactcagcaacattctggagaaaagccaaggaaggacttcaggaggggagtttcccccttctcagggcagaattttaatctccagaccaacaagaagttccctaatgtggattgaaaggctaatgaggtttatttttaactactttctatttgtttgaatgttgcatatttctactagtgaaattttcccttaataaagccattaatacacc

SEQ ID NO: 54 - protein, 648 aa, Homo sapiens
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGAAVVVSERILRVFGVSEWLP

SEQ ID NO: 55 - DNA, 2627 bp, Homo sapiens
gcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcagctgttgttgtttctgaaagaatcttgagggtgtttggagtctcagaatggcttccttaaagactaccttcagactctcagctgctcatccacaacagagatcagcctttctttgtagatgattcattcctggctgcatttgaaaaccacatattgttaattgcttgacgaatttaaatcccttgactacttttcatttcaaaaaaaaaaaaaaaa

SEQ ID NO: 56 - protein, 572 aa, Homo sapiens
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRNTRRKRLWKLIIRSINSCICSPRETEVPVRQQK

SEQ ID NO: 57 - DNA, 3729 bp, Homo sapiens
gcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgaaatacccgaagaaagagactctggaaactcattatcaggtctatcaactcttgtatttgttctcccagggaaacagaagtacctgtgcgccagcagaaatgattgcactattgataaattccgaaggaaaaattgtccatcttgtcgtcttcggaaatgttatgaagcagggatgactctgggagaaaaattccgggttggcaattgcaagcatctcaaaatgaccagaccctgaagaaaggctgacttgcctcattcaaaatgagggctctagagggctctagtggatagtctggagaaacctggcgtctgaggcttaggagcttaggtttttgctcctcaacacagactttgacgttggggttgggggctactctcttgattgctgactccctccagcgggaccaatagtgttttcctacctcacagggatgttgtgaggacgggctgtagaagtaatagtggttaccattcatgtagttgtgagtatcatgattattgtttcctgtaatgtggcttggcattggcaaagtgctttttgattgttcttgatcacatatgatgggggccaggcactgactcaggcggatgcagtgaagctctggctcagtcgcttgcttttcgtggtgtgctgccaggaagaaactttgctgatgggactcaaggtgtcaccttggacaagaagcaactgtgtctgtctgaggttcctgtggccatctttatttgtgtattaggcaattcgtatttcccccttaggttctagccttctggatcccagccagtgacctagatcttagcctcaggccctgtcactgagctgaaggtagtagctgatccacagaagttcagtaaacaaggaccagatttctgcttctccaggagaagaagccagccaacccctctcttcaaacacactgagagactacagtccgactttccctcttacatctagccttactgtagccacactccttgattgctctctcacatcacatgcttctcttcatcagttgtaagcctctcattcttctcccaagccagactcaaatattgtattgatgtcaaagaagaatcacttagagtttggaatatcttgttctctctctgctccatagcttccatattgacaccagtttctttctagtggagaagtggagtctgtgaagccagggaaacacacatgtgagagtcagaaggactctccctgacttgcctggggcctgtctttcccaccttctccagtctgtctaaacacacacacacacacacacacacacacacacacgctctctctctctctccccccccaacacacacacactctctctctctctcacacacacacacatacacacacacttctttctctttcccctgactcagcaacattctggagaaaagccaaggaaggacttcaggaggggagtttcccccttctcagggcagaattttaatctccagaccaacaagaagttccctaatgtggattgaaaggctaatgaggtttatttttaactactttctatttgtttgaatgttgcatatttctactagtgaaattttcccttaataaagccattaatacacc

SEQ ID NO: 58 - protein, 899 aa, Mus musculus
MEVQLGLGRVYPRPPSKTYRGAFQNLFQSVREAIQNPGPRHPEAANIAPPGACLQQRQETSPRRRRRQQHTEDGSPQAHIRGPTGYLALEEEQQPSQQQAASEGHPESSCLPEPGAATAPGKGLPQQPPAPPDQDDSAAPSTLSLLGPTFPGLSSCSADIKDILNEAGTMQLLQQQQQQQQHQQQHQQHQQQQEVISEGSSARAREATGAPSSSKDSYLGGNSTISDSAKELCKAVSVSMGLGVEALEHLSPGEQLRGDCMYASLLGGPPAVRPTPCAPLPECKGLPLDEGPGKSTEETAEYSSFKGGYAKGLEGESLGCSGSSEAGSSGTLEIPSSLSLYKSGALDEAAAYQNRDYYNFPLALSGPPHPPPPTHPHARIKLENPLDYGSAWAAAAAQCRYGDLGSLHGGSVAGPSTGSPPATTSSSWHTLFTAEEGQLYGPGGGGGSSSPSDAGPVAPYGYTRPPQGLTSQESDYSASEVWYPGGVVNRVPYPSPNCVKSEMGPWMENYSGPYGDMRLDSTRDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGENSNAGSPTEDPSQKMTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 59 - DNA, 10063 bp, Mus musculus
cagcgccccctcggagatccctaggagccagcctgctgggagaaccagagggtccggagcaaacctggaggctgagagggcatcagaggggaaaagactgagctagccactccagtgccatacagaagcttaagggacgcaccacgccagccccagcccagcgacagccaacgcctgttgcagagcggcggcttcgaagccgccgcccaggagctgccctttcctcttcggtgaagtttctaaaagctgcgggagactcagaggaagcaaggaaagtgtccggtaggactacggctgcctttgtcctcttcccctctacccttaccccctcctgggtcccctctccaggagctgactaggcaggctttctggccaaccctctcccctacacccccagctctgccagccagtttgcacagaggtaaactccctttggctgagagtaggggagcttgttgcacattgcaaggaaggcttttgggagcccagagactgaggagcaacagcacgcccaggagagtccctggttccaggttctcgcccctgcacctcctcctgcccgcccctcaccctgtgtgtggtgttagaaatgaaaagatgaaaaggcagctagggtttcagtagtcgaaagcaaaacaaaagctaaaagaaaacaaaaagaaaatagcccagttcttatttgcacctgcttcagtggactttgaatttggaaggcagaggatttccccttttccctcccgtcaaggtttgagcatcttttaatctgttcttcaagtatttagagacaaactgtgtaagtagcagggcagatcctgtcttgcgcgtgccttcctttactggagactttgaggttatctgggcactccccccacccaccccccctcctgcaagttttcttccccggagcttcccgcaggtgggcagctagctgcagatactacatcatcagtcaggagaactcttcagagcaagagacgaggaggcaggataagggaattcggtggaagctacagacaagctcaaggatggaggtgcagttagggctgggaagggtctacccacggcccccatccaagacctatcgaggagcgttccagaatctgttccagagcgtgcgcgaagcgatccagaacccgggccccaggcaccctgaggccgctaacatagcacctcccggcgcctgtttacagcagaggcaggagactagcccccggcggcggcggcggcagcagcacactgaggatggttctcctcaagcccacatcagaggccccacaggctacctggccctggaggaggaacagcagccttcacagcagcaggcagcctccgagggccaccctgagagcagctgcctccccgagcctggggcggccaccgctcctggcaaggggctgccgcagcagccaccagctcctccagatcaggatgactcagctgccccatccacgttgtccctgctgggccccactttcccaggcttaagcagctgctccgccgacattaaagacattttgaacgaggccggcaccatgcaacttcttcagcagcagcaacaacagcagcagcaccaacagcagcaccaacagcaccaacagcagcaggaggtaatctccgaaggcagcagcgcaagagccagggaggccacgggggctccctcttcctccaaggatagttacctagggggcaattcaaccatatctgacagtgccaaggagttgtgtaaagcagtgtctgtgtccatgggattgggtgtggaagcattggaacatctgagtccaggggaacagcttcggggagactgcatgtacgcgtcgctcctgggaggtccacccgcggtgcgtcccactccttgtgcgccgctgcccgaatgcaaaggtcttcccctggacgaaggcccaggcaaaagcactgaagagactgctgagtattcctctttcaagggaggttacgccaaaggattggaaggtgagagcttggggtgctctggcagcagtgaagcaggtagctctgggacacttgagatcccgtcctctctgtctctgtataaatctggagcactagacgaggcagcagcataccagaatcgcgactactacaactttccgctggctctgtccgggccgccgcaccccccgccccctacccatccacacgcccgtatcaagctggagaacccattggactacggcagcgcctgggctgcggcggcagcgcaatgccgctatggggacttgggtagtctacatggagggagtgtagccgggcccagcactggatcgcccccagccaccacctcttcttcctggcatactctcttcacagctgaagaaggccaattatatgggccaggaggcgggggcggcagcagcagcccaagcgatgccgggcctgtagccccctatggctacactcggccccctcaggggctgacaagccaggagagtgactactctgcctccgaagtgtggtatcctggtggagttgtgaacagagtaccctatcccagtcccaattgtgtcaaaagtgaaatgggaccttggatggagaactactccggaccttatggggacatgcgtttggacagtaccagggaccatgttttacccatcgactattactttccaccccagaagacctgcctgatctgtggagatgaagcttctggctgtcactacggagctctcacttgtggcagctgcaaggtcttcttcaaaagagccgctgaagggaaacagaagtatctatgtgccagcagaaacgattgtaccattgataaatttcggaggaaaaattgcccatcttgtcgtctccggaaatgttatgaagcagggatgactctgggagctcgtaagctgaagaaacttggaaatctaaaactacaggaggaaggagaaaactccaatgctggcagccccactgaggacccatcccagaagatgactgtatcacacattgaaggctatgaatgtcagcctatctttcttaacgtcctggaagccattgagccaggagtggtgtgtgccggacatgacaacaaccaaccagattcctttgctgccttgttatctagcctcaatgagcttggagagaggcagcttgtgcatgtggtcaagtgggccaaggccttgcctggcttccgcaacttgcatgtggatgaccagatggcggtcattcagtattcctggatgggactgatggtatttgccatgggttggcggtccttcactaatgtcaactccaggatgctctactttgcacctgacttggttttcaatgagtaccgcatgcacaagtctcggatgtacagccagtgtgtgaggatgaggcacctgtctcaagagtttggatggctccaaataaccccccaggaattcctgtgcatgaaagcactgctgctcttcagcattattccagtggatgggctgaaaaatcaaaaattctttgatgaacttcgaatgaactacatcaaggaactcgatcgcatcattgcatgcaaaagaaagaatcccacatcctgctcaaggcgcttctaccagctcaccaagctcctggattctgtgcagcctattgcaagagagctgcatcagttcacttttgacctgctaatcaagtcccatatggtgagcgtggactttcctgaaatgatggcagagatcatctctgtgcaagtgcccaagatcctttctgggaaagtcaagcccatctatttccacacacagtgaagatttggaaaccctaatacccaaaacccaccttgttccctttccagatgtcttctgcctgttatataactctgcactacttctctgcagtgccttgggggaaattcctctactgatgtacagtctgtcgtgaacaggttcctcagttctatttcctgggcttctccttctttttttttcttcttccctccctctttcaccctcccatggcacattttgaatctgctgcgtattgtggctcctgcctttgttttgatttctgttgtatttctttgaatctgtgatgatcctcttgtggcccagtgtcaattgtgcttgtttatagcactgtgctgtgtgccaaccaagcaaatgtttactcaccttatgccatggcaaatttagagagctataagtatctggagaagaaacaaacagagagaataaaaagcaaaaacaaaaccaaaaaataaaaaaaacacaaacaaaaaacaaaaccaacaaacaaaacatgctaggtttgtttcttcgtggtatacaaataaacacataggattcccaaagaagccgacagtgactagaagaaagtaaaaaattacaaatccacgaggagtcactgtttttgttcatcctgtttctctgtgggaaacttcagttgttgttaatggctattgccattaaagagcaggttgaccccaaagctttactgatagggtagagagaaaagaggacaaggagggcagatggataaccattacctccccacagcctttgtccctgagtcctagagtgctcagttgcagtgtagttccttgtactgaaatgtgcttcttgtttgaaaacttgtctgcatgtgaatgcctcttccttccaatccttttctctcttaacctctgcttccaccctcaattgactttcaatagcttttctcagagctttgtactatatgctctctttagccaaaacttggccactttcactgaagttatgtcagtgagaagaaagtggaaaggtctgactctttggaaggctctattcagatttatgttcatatttccatgtgtgagccatagcggagctttgtgactggagtcagaggaaaaggaagtgatggcttagccattctcccattagagatagtgaatgatgatgccatagtgcaatcatcctttcctctgcttttaaaggacctagagaccccatgcagccacattctccctgcacaagtcttcagtgttcagtggccctgaacttcaccaaaatgcatttaagccaaggtggtaaagcttgtacacttctttggacgtgtttgtagacactgctaagatctccctctcaccaccaccacaaaggctagcaggccagcagccacagcatctatgtttagatgttaatagcataaaagacatctcactcaatgtctttcatcaacagtaaatttctggagcccttagaaaaattggaaagaaagcatcaaagggaccagacaaaatgggcatcttgcccttgtcctccagagacaatatattcctcccaagtggagaaatgtcaatttcctcctcagaacaattaaaggggctacccagaccatggtggaagagaaaactaagtaacccagctgagaaaaatgaagacactagaaccagaaagcacaggactttttcctttccatccagcatacccattggcagaaataatggaaggaaaagagaaggccagaagaaaatacagactgctgaagtcttcagaggcaaagtctaaagccagatgaataccatctggctagatgggcatcagtttgctcatcctcctctattgccattgctgggctgactttggccaaagttacttcgaatctccaccatagttgtcccctctcagtcagagggtgcaggaccactgaaacattctatccaccgtgactctcattggacagatctggccggtgtggctacaaatagactgcacccataaactcagggcaagccctgggtcactggtttcatgtagtctgttgacagccttctttactgtggactctgttcctcaaccttgagtgcaggaggaatgcacatctacttttgcctttgtatcattcctcctcactcagctcttcacctccctgcagaccttaagaaatcaggggccagctgccaagctgactcttttggttggtactatgttaactgaaaaggtgatttccgaaggacaggttttcttccctgatttctttgttgctattaatagcaaaaacaaacttgcaaaacaacttctttaacaaggaagggaggatatatacaatgggtgatatggtaatccaaccctgcttgacaaaaactgaagctgacaggttacatttaaaaacaaaacaaaacaaaacgggacagtttctgatttgctttgtgacaacaccatctggcttatgtacaggagctctcttagctgttccttaaacagaaaaaaaatcattactccttttagttaaatttggttacattttaatagtttctttacatctattctgaagcaatttttgtcttctgtggtacatggattttattataacattctaatatttgtctttgtaaatactagagactctttgatccatttctctaggaagtttttcatcttatggagttctgaatcatgacttttatctttatgaatgtatatgctttttacttgcaaaagccaaaaagagtgaaacagcagtgcaattaaagcaacaccaactaaactccaaatttccaagtgacaatattagagaaaaacagcatacacatggctttatgcctactgcttctgcggtggggtttgggtgcgcaatggaaactgtagcttggctgtgttctcccacacaagtgaagaagagattggtttttgcttttttggattttgtgtttcttttctgttttgttttgttttgttttgttttgctttgctttctttggccatcaatgttccaactaatatgattggcggagcacgtgctctgctcagtagagtgaatgttgctggtgcactatgctcacctgtgaacggctggccatttctccattcatatggttaagatggaagatgaggatcacttaccagagaagtcaaggtgatcatctccaaagaggtttacagtgcttggtaggaatggaaaatgaggacaagaaaaagaggagaaccatggagaaggcccaactgggcaggacagcagccagctgccaaagtcacgaactctgggattcaagaagagtcgtgtagtgctttcaactctcatccgcaggcagctcactgtgtgtggactctgagctgacacgggagttggcttctttgttccatagattttctatgccacaggcaatattattgttcttggaaagttcattatttttttaaattaccttactctcagaaagggatttttttgaaggattctgtcatatatctttggaaaacagaaaatcagtaatatgtatatttttatgtatgttcactggcactaaaaaaaaaaaaaaaaagaaaagaaaaaaaagagaaaaaaaaaagcttcactctgtcctttgggtagttgctgaggttaattgtccaggttgagaaatgtgcttctgctaacatccttctctgtccacactctatttctaagtacatataggcatatataggaagatatattcaacacactttaagaaaaaagtatgtccaccatccacatgataaccacaatgatactccacaaattacatgactttaagcttcaagcaacttctaactgattcattcatttatagccttgccctcttctttcccttaaatttggcccagcacaaagacccaagccacccttatacctccctaagacttaagccagcaccagacttcagaaggttttctgaagacaactgacttgctatccctgcatgaccctagcatggtcctgcaaacacaagagactaattataattctcctccactaattgcctgggtcacaggtcattgggccaaggccatgattcttatgcttacgaaccactaatgctaacctactagattaaatcctgaactgaaagttaaaagaagccatttagcatgtgaaacttcttggagtaagaagtttctgtcccggctgcctttgcaaacaggtttgctttcaccacttatctccttgaaaatctttgaaggccttttttttttaagtagaaaaggagatgaaagcattatattatgtaaccaaagattatattgtatctaagataccaaattttttaagggcagggaaggagcaagcattagtgcctctttggtaaattatccaaagacagactgaaggacttttctgatgattgacttagaagactttgtggggaggggttgtctcacaatatacatatttagaagtgttgagaataatttggggggaaatgggattatagtgtccttcactaactgattttataagcagaactagctttccttttttttttttttaaagtagttacaaagcaaattcttaaagctccatctttgcatggttagaaatggagctggtcttggccactgtgtttactagtgcccatgttagcttatttgaagatgtgaagcccttgataagaaggggtacatttaaaggattagatttttgcactagaaggagggcaggcagaaaccctcatttctgcccagtttggacagcacaaaaagttctctgcagtttaaggcagaaagttgaaatatattgtaaatgagtatttgtatccatgtttcaaaactgaattctatatatagatgtaatgtgttctgatagctttacctttctctgcacctttatatttggttccaggtcatatctgatgccatgtacttgtaagagaggttgcagttacatttttggatgctctctcagaatggataagacacctggattgatcagataactgagatctcttcccttcttgggcctggtgttgaggccttgcaaaggggtggaagaggaaagggtagggtacatgatgtattgcactttactagcttaagacggatgaatgtggaaagggtggtgaaatttcattgaaaatgcctaggaattgcaatagggagaaatccagatgtggggccaggtgcccacccaaaggactggccagcagcctcttcatgggatctgaggcattgggaaaaggaaggctatttccttggttttcaccatccttgttagagaagggcagttgcctggtcttgggaacctggagcaaacgctccttctgtcacatcaattctttcccctgcaattgaggtgctcttgctactgggtgtccgtgtgctctaattctggttctggatatgttctgtaaagattttgataattgctaatgtatttttctctgttaaaaatttgttagtgtgttagaagtcatatctctgtaggtacagatcctttgctacccatgagtagagggattttttttcttcaattaagagtttgaccctggggtctgttgcccagagcccatccagaaaaaaaaatccacatttgtcacaatttttctgaaatttcagtcaaggtaacagatcgctgggagttctctttacccccccaaaaaagcagataattgaatttagcaggtggtgttttagagcaaaaaacaaaacagcctttgacccagctttaatatgacccaatttaatctggccaggaagcaggtaatgtgtattaattggcttccaatcctggttgagtgtagcaaggttctactttgtttcctagttccttttgttacatggcctttcacagaaaggattgactgggtttgcagtatatcttatggccttagcacctattgctaactgtcctgaagggaattgcctatggggttgtcctataagccacttctatcattaaaagcagccaccaatggaatctcccaggtttgaaaaaaaaaaaaaaaaacagatggtcctttaccattcattgacacacatccctgctttcctgtagacagattgactggacattgattagggaatacatggcaaatgacatgcttacactaccctggagattaatttggcagtaggagggaatagacaatgtaaccaagaatgtaatgtaattcttatagagataagaattaaatctggatgtggagagagcaaagagagaaagcattcaatttttttttcaaaagaaaccaatttattttgcttgaaacttctttcgctggggcttcagttctcacagcggctcttggtctccactgggcagcaggaccagccccaagcgctagtgttctgttctctttttgtaatcttggaatcttttgttgctctaaatacaattaaaaatggcagaaacttgtttgttggaatac